(19) United States
(12) Patent Application Publication
(10) Pub. No.: US 2025/0127907 A1
(43) Pub. Date: Apr. 24, 2025

(54) PARP INHIBITORS

(71) Applicant: Stablix, Inc., Boston, MA (US)

(72) Inventors: Eddine Saiah, Boston, MA (US); Tyler Mann, Boston, MA (US); Jorden Kass, Arlington, MA (US); Louise Clare Kirman, Swampscott, MA (US); Mark George Saulnier, Higganum, CT (US); Lisa A. De Meese, Silver Spring, MD (US); Richard James Steel, Foster City, CA (US); Kap-Sun Yeung, Boston, MA (US)

(21) Appl. No.: 18/752,730

(22) Filed: Jun. 24, 2024

Related U.S. Application Data
(60) Provisional application No. 63/510,040, filed on Jun. 23, 2023.

(51) Int. Cl. A61K 47/55

(52) U.S. Cl. CPC A61K 47/55

(57) ABSTRACT

The present invention provides compounds, compositions thereof, and methods of using the same.

### CROSS-REFERENCE TO RELATED APPLICATIONS

This application claims the benefit of priority to U.S. Provisional Appl. No. 63/510,040, filed Jun. 23, 2023, the content of which is herein incorporated by reference.

### TECHNICAL FIELD OF THE INVENTION

This invention provides bifunctional molecules that modulate biological function by inducing proximity between a deubiquitinase (DUB) and a target of interest, compositions, and methods of use thereof. The bifunctional molecules include a deubiquitinase targeting ligand, a protein targeting ligand, and optionally a linker that connects the two for the modulation of a target protein to treat a disorder.

### BACKGROUND OF THE INVENTION

The ubiquitination of proteins is a dynamic multifaceted post-translational modification that allows the body to mark proteins for degradation, activation, sub-cellular localization, and translocation. The best-characterized function of ubiquitin is as a marker for protein degradation. However, protein deubiquitination can result in outcomes other than preventing degradation, stabilization and enhancement of protein levels. The specific lysine being ubiquitinated influences whether the ubiquitinated protein is targeted for proteasomal degradation, activation or trafficking. For example, the most common linkage, on K48, is closely associated with degradation by the proteasome. K63-linked ubiquitination is associated with pathway activation, protein trafficking, DNA repair, and inflammation. Monoubiquitination also is not typically associated with degradation, and in some cases may regulate protein function by impeding or facilitating protein-protein association.

A deubiquitinase protein (“DUB”) is a protein that partially or fully removes ubiquitin molecules from proteins. Deubiquitinases (DUBs) can be classified into six main classes: ubiquitin-specific proteases (USPs), ubiquitin C-terminal hydrolases (UCHs), Machado-Joseph disease protein domain proteases (MJDs), ovarian tumor proteases (OTUs), JAB1/MPN/MOV34 metalloenzymes (JAMMs), and motif interacting with ubiquitin-containing novel DUB family (MINDY) DUBs (Mevissen et. al., “Mechanisms of Deubiquitinase Specificity and Regulation” Annu. Rev. Biochem. 2017 (86) 159).

Bifunctional compounds composed of a target protein-binding ligand and a DUB, induced deubiquitination of selected proteins via their recruitment to the DUB, E3 ubiquitin ligase and subsequent ubiquitination. Bifunctional molecules for targeted protein deubiquitination are described in WO2021/146386A1, WO2020/169650 and Henning, Nathaniel J et al. “Deubiquitinase-targeting chimeras for targeted protein stabilization.” Nature chemical biology vol. 18,4 (2022): 412-421. doi:10.1038/s41589-022-00971-2.

Poly(ADP-ribose) polymerase 1 (PARP1) is an abundant chromatin-associated enzyme, whose activity is strongly induced in response to genotoxic stress (Gibson and Kraus, 2012; Ray Chaudhuri and Nussenzweig, 2017; Teloni and Altmeyer, 2016). PARP1 has affinity for DNA single- and double-strand breaks (DSBs), for stalled and reversed replication forks, and for a range of atypical DNA secondary structures (Alemasova and Lavrik, 2019; Neelsen and Lopes, 2015). Upon DNA binding and allosteric activation, PARP1 uses the energy carrier nicotinamide adenine dinucleotide (NAD+) as substrate to generate poly(ADP-ribose) (PAR) chains (Gupte et al., 2017; Eustermann et al., 2015; Hottiger, 2015). PAR signaling promotes DNA repair reactions and helps maintain genome stability.

PARP inhibitors (PARPi) are used in the clinic for the treatment of a variety of cancers and cause synthetic lethality in BRCA1/2-deficient tumors (Lord and Ashworth, 2017; O'Connor, 2015). The tumor suppressors BRCA1 and BRCA2 are critical components of the homology-directed repair (HDR) pathway, which promotes error-free repair of DNA double-strand breaks (DSBs) and emerges as an important contributor to replication fork stability (Chen et al., 2018). In a number of heritable and sporadic breast and ovarian cancers BRCA1/2 function is lost, and this situation generates a cancer-specific vulnerability to PARPi (Lord and Ashworth, 2017; O'Connor, 2015).

Vulnerabilities to PARPi may also exist in HDR-proficient cancer cells. By blocking access of NAD+ to the catalytic site, PARPi lock PARP1 in an inactive cytotoxic conformation, a situation referred to as PARP trapping (Murai et al., 2012; Pommier et al., 2016). Consistent with PARP trapping being an important cause of PARPi toxicity in BRCA-proficient cells, loss of PARP1 rescues them from PARPi-induced genotoxic stress and cell death (Michelena et al., 2018; Murai et al., 2012). Thus the inactivated trapped PARP1 plays a role in the anticancer activity of PARPi.

It was shown recently (Cell Rep. 2020 Aug. 4; 32(5): 107985) that PARP ubiquitination by the ligase TRIP12 results in degradation and reduced trapping. PARP ubiquitination limits PARPi-induced cytotoxic PARP1 trapping. Blocking the ligase activity results in reduced PARP degradation and increased PARPi-induced PARP1 trapping ultimately resulting in increased DNA replication stress, DNA damage, cell cycle arrest, and cell death. In cohorts of breast and ovarian cancer patients, PARP1 abundance is negatively correlated with TRIP12 ligase expression. These results highlight the critical role of the PARP pathway in cancer development. Accordingly, there remains a need to find compounds that are PARP inhibitors useful as therapeutic agents.

### SUMMARY OF THE INVENTION

The present application relates to novel bifunctional compounds, which function to recruit PARP protein to DUBs for deubiquitination, and methods of preparation and uses thereof. In particular, the present disclosure provides bifunctional compounds, which find utility as inducers of targeted deubiquitination of PARP resulting in inhibition.

It has now been found that compounds of this invention, and pharmaceutically acceptable compositions thereof, are effective as inhibitors of PARP protein. Such compounds have the general formula I:

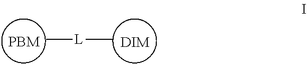

or a pharmaceutically acceptable salt thereof, wherein each variable is as defined and described herein.

Compounds of the present invention, and pharmaceutically acceptable compositions thereof, are useful for treating a variety of diseases, disorders or conditions, associated with regulation of PARP protein. Such diseases, disorders, or conditions include those described herein.

### DETAILED DESCRIPTION OF SOME EMBODIMENTS

### General Description of Some Embodiments of the Invention

Compounds of the present invention, and compositions thereof, are useful as deactivators and/or trapping compounds and/or inhibitors of PARP protein. In some embodiments, a provided compound deactivates and/or traps and/or inhibits PARP protein.

In some embodiments, the present invention provides a compound of formula I:

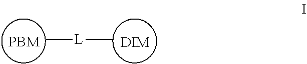

or a pharmaceutically acceptable salt thereof, wherein:
PBM is a protein targeting moiety capable of binding PARP;
L is a bivalent moiety that connects PBM to DIM; and
DIM is a deubiquitination inducing moiety, such as a deubiquitinase binding moiety (DBM).

### Compounds and Definitions

The terms PARP protein and PARP refer to all members of the poly ADP ribose polymerase (PARP) family, including, but not limited to PARP1, PARP2, PARP3, PARP4 (VPARP), PARP-5a (Tankyrase-1, TNKS), PARP-5b (Tankyrase-2, TNKS2), PARP6, PARP7 (TIPARP), PARP8, PARP9, PARP10, PARP11, PARP12, PARP 13, PARP14, PARP15, PARP16 and PARP17.

Compounds of the present invention include those described generally herein, and are further illustrated by the classes, subclasses, and species disclosed herein. As used herein, the following definitions shall apply unless otherwise indicated. For purposes of this invention, the chemical elements are identified in accordance with the Periodic Table of the Elements, CAS version, Handbook of Chemistry and Physics, 75^{th} Ed. Additionally, general principles of organic chemistry are described in “Organic Chemistry”, Thomas Sorrell, University Science Books, Sausalito: 1999, and “March's Advanced Organic Chemistry”, 5^{th} Ed., Ed.: Smith, M. B. and March, J., John Wiley & Sons, New York: 2001, the entire contents of which are hereby incorporated by reference.

The term “aliphatic” or “aliphatic group”, as used herein, means a straight-chain (i.e., unbranched) or branched, substituted or unsubstituted hydrocarbon chain that is completely saturated or that contains one or more units of unsaturation, or a monocyclic, bicyclic, bridged bicyclic, or spirocyclic hydrocarbon that is completely saturated or that contains one or more units of unsaturation, but which is not aromatic (also referred to herein as “carbocycle,” “cycloaliphatic” or “cycloalkyl”), that has a single point of attachment to the rest of the molecule. Unless otherwise specified, aliphatic groups contain 1-6 aliphatic carbon atoms. In some embodiments, aliphatic groups contain 1-5 aliphatic carbon atoms. In other embodiments, aliphatic groups contain 1-4 aliphatic carbon atoms. In still other embodiments, aliphatic groups contain 1-3 aliphatic carbon atoms, and in yet other embodiments, aliphatic groups contain 1-2 aliphatic carbon atoms. In some embodiments, “cycloaliphatic” (or “carbocycle” or “cycloalkyl”) refers to a monocyclic C₃-C₆ hydrocarbon that is completely saturated or that contains one or more units of unsaturation, but which is not aromatic, that has a single point of attachment to the rest of the molecule. Suitable aliphatic groups include, but are not limited to, linear or branched, substituted or unsubstituted alkyl, alkenyl, alkynyl groups and hybrids thereof such as (cycloalkyl)alkyl, (cycloalkenyl)alkyl or (cycloalkyl)alkenyl.

As used herein, the term “bridged bicyclic” refers to any bicyclic ring system, i.e. carbocyclic or heterocyclic, saturated or partially unsaturated, having at least one bridge. As defined by IUPAC, a “bridge” is an unbranched chain of atoms or an atom or a valence bond connecting two bridgeheads, where a “bridgehead” is any skeletal atom of the ring system which is bonded to three or more skeletal atoms (excluding hydrogen). In some embodiments, a bridged bicyclic group has 7-12 ring members and 0-4 heteroatoms independently selected from nitrogen, oxygen, or sulfur. Such bridged bicyclic groups are well known in the art and include those groups set forth below where each group is attached to the rest of the molecule at any substitutable carbon or nitrogen atom. Unless otherwise specified, a bridged bicyclic group is optionally substituted with one or more substituents as set forth for aliphatic groups. Additionally or alternatively, any substitutable nitrogen of a bridged bicyclic group is optionally substituted. Exemplary bridged bicyclics include:

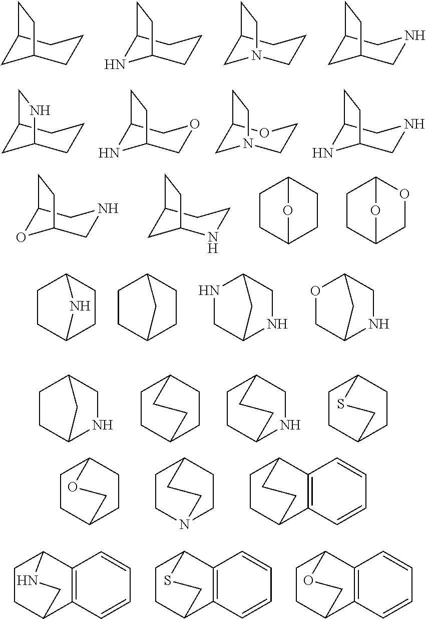

The term “lower alkyl” refers to a C₁₋₄ straight or branched alkyl group. Exemplary lower alkyl groups are methyl, ethyl, propyl, isopropyl, butyl, isobutyl, and tert-butyl.

The term “lower haloalkyl” refers to a C₁₋₄ straight or branched alkyl group that is substituted with one or more halogen atoms.

The term “heteroatom” means one or more of oxygen, sulfur, nitrogen, phosphorus, or silicon (including, any oxidized form of nitrogen, sulfur, phosphorus, or silicon; the quaternized form of any basic nitrogen or; a substitutable nitrogen of a heterocyclic ring, for example N (as in 3,4-dihydro-2H-pyrrolyl), NH (as in pyrrolidinyl) or NR (as in N-substituted pyrrolidinyl).

The term “unsaturated,” as used herein, means that a moiety has one or more units of unsaturation.

As used herein, the term “bivalent C₁₋₈ (or C₁₋₆) saturated or unsaturated, straight or branched, hydrocarbon chain”, refers to bivalent alkylene, alkenylene, and alkynylene chains that are straight or branched as defined herein.

The term “alkylene” refers to a bivalent alkyl group. An “alkylene chain” is a polymethylene group, i.e., —(CH₂)ₙ—, wherein n is a positive integer, preferably from 1 to 6, from 1 to 4, from 1 to 3, from 1 to 2, or from 2 to 3. A substituted alkylene chain is a polymethylene group in which one or more methylene hydrogen atoms are replaced with a substituent. Suitable substituents include those described below for a substituted aliphatic group.

The term “alkenylene” refers to a bivalent alkenyl group. A substituted alkenylene chain is a polymethylene group containing at least one double bond in which one or more hydrogen atoms are replaced with a substituent. Suitable substituents include those described below for a substituted aliphatic group.

As used herein, the term “cyclopropylenyl” refers to a bivalent cyclopropyl group of the following structure:

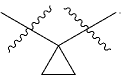 [SMILES: *C1(*)CC1]

The term “halogen” means F, Cl, Br, or I.

The term “aryl” used alone or as part of a larger moiety as in “aralkyl,” “aralkoxy,” or “aryloxyalkyl,” refers to monocyclic or bicyclic ring systems having a total of five to fourteen ring members, wherein at least one ring in the system is aromatic and wherein each ring in the system contains 3 to 7 ring members. The term “aryl” may be used interchangeably with the term “aryl ring.” In some embodiments of the present invention, “aryl” refers to an aromatic ring system which includes, but not limited to, phenyl, biphenyl, naphthyl, anthracyl and the like, which may bear one or more substituents. Also included within the scope of the term “aryl,” as it is used herein, is a group in which an aromatic ring is fused to one or more non-aromatic rings, such as indanyl, phthalimidyl, naphthimidyl, phenanthridinyl, or tetrahydronaphthyl, and the like.

The terms “heteroaryl” and “heteroar-,” used alone or as part of a larger moiety, e.g., “heteroaralkyl,” or “heteroaralkoxy,” refer to groups having 5 to 10 ring atoms, preferably 5, 6, or 9 ring atoms; having 6, 10, or 14 π electrons shared in a cyclic array; and having, in addition to carbon atoms, from one to five heteroatoms. The term “heteroatom” refers to nitrogen, oxygen, or sulfur, and includes any oxidized form of nitrogen or sulfur, and any quaternized form of a basic nitrogen. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl. The terms “heteroaryl” and “heteroar-”, as used herein, also include groups in which a heteroaromatic ring is fused to one or more aryl, cycloaliphatic, or heterocyclyl rings, where the radical or point of attachment is on the heteroaromatic ring. Nonlimiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, and pyrido[2,3-b]-1,4-oxazin-3(4H)-one. A heteroaryl group may be mono- or bicyclic. A heteroaryl ring may include one or more oxo (═O) or thioxo (═S) substituent. The term “heteroaryl” may be used interchangeably with the terms “heteroaryl ring,” “heteroaryl group,” or “heteroaromatic,” any of which terms include rings that are optionally substituted. The term “heteroaralkyl” refers to an alkyl group substituted by a heteroaryl, wherein the alkyl and heteroaryl portions independently are optionally substituted.

As used herein, the terms “heterocycle,” “heterocyclyl,” “heterocyclic radical,” and “heterocyclic ring” are used interchangeably and refer to a stable 5-to 7-membered monocyclic or 7-10-membered bicyclic heterocyclic moiety that is either saturated or partially unsaturated, and having, in addition to carbon atoms, one or more, preferably one to four, heteroatoms, as defined above. When used in reference to a ring atom of a heterocycle, the term “nitrogen” includes a substituted nitrogen. As an example, in a saturated or partially unsaturated ring having 0-3 heteroatoms selected from oxygen, sulfur or nitrogen, the nitrogen may be N (as in 3,4-dihydro-2H-pyrrolyl), NH (as in pyrrolidinyl), or ⁺NR (as in N-substituted pyrrolidinyl).

A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothiophenyl pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl. The terms “heterocycle,” “heterocyclyl,” “heterocyclyl ring,” “heterocyclic group,” “heterocyclic moiety,” and “heterocyclic radical,” are used interchangeably herein, and also include groups in which a heterocyclyl ring is fused to one or more aryl, heteroaryl, or cycloaliphatic rings, such as indolinyl, 3H-indolyl, chromanyl, phenanthridinyl, or tetrahydroquinolinyl. A heterocyclyl group may be monocyclic, bicyclic, bridged bicyclic, or spirocyclic. A heterocyclic ring may include one or more oxo (═O) or thioxo (═S) substituent. The term “heterocyclylalkyl” refers to an alkyl group substituted by a heterocyclyl, wherein the alkyl and heterocyclyl portions independently are optionally substituted.

As used herein, the term “partially unsaturated” refers to a ring moiety that includes at least one double or triple bond. The term “partially unsaturated” is intended to encompass rings having multiple sites of unsaturation, but is not intended to include aryl or heteroaryl moieties, as herein defined.

As described herein, compounds of the invention may contain “optionally substituted” moieties. In general, the term “substituted” means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. The term “stable,” as used herein, refers to compounds that are not substantially altered when subjected to conditions to allow for their production, detection, and, in some embodiments, their recovery, purification, and use for one or more of the purposes disclosed herein.

Suitable monovalent substituents on a substitutable carbon atom of an “optionally substituted” group are independently halogen; —(CH₂)₀₋₄R^{∘}; —(CH₂)₀₋₄OR^{∘}; —O(CH₂)₀₋₄R^{∘}, —O—(CH₂)₀₋₄C(O)OR^{∘}; —(CH₂)₀₋₄CH(OR^{∘})₂; —(CH₂)₀₋₄SR^{∘}; —(CH₂)₀₋₄Ph, which may be substituted with R^{∘}; —(CH₂)₀₋₄O(CH₂)₀₋₁Ph which may be substituted with R^{∘}; —CH═CHPh, which may be substituted with R^{∘}; —(CH₂)₀₋₄O(CH₂)₀₋₁-pyridyl which may be substituted with R^{∘}; —NO₂; —CN; —N₃; —(CH₂)₀₋₄N(R^{∘})₂; —(CH₂)₀₋₄N(R^{∘})C(O)R^{∘}; —N(R^{∘})C(S)R^{∘}; —(CH₂)₀₋₄N(R^{∘})C(O)NR^{∘}₂; —N(R^{∘})C(S)NR^{∘}₂; —(CH₂)₀₋₄N(R^{∘})C(O)OR^{∘}; —N(R^{∘})N(R^{∘})C(O)R^{∘}; —N(R^{∘})N(R^{∘})C(O)NR^{∘}₂; —N(R^{∘})N(R^{∘})C(O)OR^{∘}; —(CH₂)₀₋₄C(O)R^{∘}; —C(S)R^{∘}; —(CH₂)₀₋₄C(O)OR^{∘}; —(CH₂)₀₋₄C(O)SR^{∘}; —(CH₂)₀₋₄C(O)OSiR^{∘}₃; —(CH₂)₀₋₄OC(O)R^{∘}; —OC(O)(CH₂)₀₋₄SR^{∘}; —(CH₂)₀₋₄SC(O)R^{∘}; —(CH₂)₀₋₄C(O)NR^{∘}₂; —C(S)NR^{∘}₂; —C(S)SR^{∘}; —SC(S)SR^{∘}, —(CH₂)₀₋₄OC(O)NR^{∘}₂; —C(O)N(OR^{∘})R^{∘}; —C(O)C(O)R^{∘}; —C(O)CH₂C(O)R^{∘}; —C(NOR^{∘})R^{∘}; —(CH₂)₀₋₄SSR^{∘}; —(CH₂)₀₋₄S(O)₂R^{∘}; —(CH₂)₀₋₄S(O)₂OR^{∘}; —(CH₂)₀₋₄OS(O)₂R^{∘}; —S(O)₂NR^{∘}₂; —(CH₂)₀₋₄S(O)R^{∘}; —N(R^{∘})S(O)₂NR^{∘}₂; —N(R^{∘})S(O)₂R^{∘}; —N(OR^{∘})R^{∘}; —C(NH)NR^{∘}₂; —(CH₂)₀₋₄P(O)₂R^{∘}; —(CH₂)₀₋₄P(O)R^{∘}₂; —(CH₂)₀₋₄P(O)(OR^{∘})₂; —(CH₂)₀₋₄OP(O)R^{∘}₂; —(CH₂)₀₋₄OP(O)(OR^{∘})₂; SiR^{∘}₃; —(C₁₋₄ straight or branched alkylene)O—N(R^{∘})₂; or —(C₁₋₄ straight or branched alkylene)C(O)O—N(R^{∘})₂, wherein each R^{∘} may be substituted as defined below and is independently hydrogen, C₁₋₃ aliphatic, —CH₂Ph, —O(CH₂)₀₋₁Ph, —CH₂-(5-6 membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of R^{∘}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{∘} (or the ring formed by taking two independent occurrences of R^{∘} together with their intervening atoms), are independently halogen, —(CH₂)₀₋₂R^{●}, -(haloR^{●}), —(CH₂)₀₋₂OH, —(CH₂)₀₋₂OR′, —(CH₂)₀₋₂CH(OR^{●})₂; —O(haloR^{●}), —CN, —N₃, —(CH₂)₀₋₂C(O)R^{●}, —(CH₂)₀₋₂C(O)OH, —(CH₂)₀₋₂C(O)OR^{●}, —(CH₂)₀₋₂SR^{●}, —(CH₂)₀₋₂SH, —(CH₂)₀₋₂NH₂, —(CH₂)₀₋₂NHR^{●}, —(CH₂)₀₋₂NR^{●}₂, —NO₂, —SiR^{●}₃, —OSiR^{●}₃, —C(O)SR^{●}, —(C₁₋₄ straight or branched alkylene)C(O)OR^{●}, or —SSR^{●} wherein each R^{●} is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{∘} include ═O and ═S.

Suitable divalent substituents on a saturated carbon atom of an “optionally substituted” group include the following: ═O, ═S, ═NNR*₂, ═NNHC(O)R*, ═NNHC(O)OR*, ═NNHS(O)₂R*, ═NR*, ═NOR*, —O(C(R*₂)₂₋₃O—, or —S(C(R*₂))₂₋₃S—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R* include halogen, —R^{●}, -(haloR*), —OH, —OR*, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on the aliphatic group of R^{●} are independently halogen, —R^{●}, -(haloR^{●}), —OH, —OR*, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Salts derived from appropriate bases include alkali metal, alkaline earth metal, ammonium and N⁺(C₁₋₄-alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate. In some embodiments, the provided compounds are purified in salt form for convenience and/or ease of purification, e.g., using an acidic or basic mobile phase during chromatography. Salts forms of the provided compounds formed during chromotagraphic purification are contemplated herein and are readily apparent to those having skill in the art.

Unless otherwise stated, structures depicted herein are also meant to include all isomeric (e.g., enantiomeric, diastereomeric, and geometric (or conformational)) forms of the structure; for example, the R and S configurations for each asymmetric center, Z and E double bond isomers, and Z and E conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention. Additionally, unless otherwise stated, structures depicted herein are also meant to include compounds that differ only in the presence of one or more isotopically enriched atoms. For example, compounds having the present structures including the replacement of hydrogen by deuterium or tritium, or the replacement of a carbon by a ¹³C- or ¹⁴C-enriched carbon are within the scope of this invention. Such compounds are useful, for example, as analytical tools, as probes in biological assays, or as therapeutic agents in accordance with the present invention

As used herein, the term “provided compound” refers to any genus, subgenus, and/or species set forth herein.

As used herein, the term “inhibitor” is defined as a compound that binds to and/or inhibits PARP with measurable affinity. In some embodiments, an inhibitor has an IC₅₀ and/or binding constant of less than about 50 μM, less than about 1 μM, less than about 500 nM, less than about 100 nM, less than about 10 nM, or less than about 1 nM.

The terms “measurable affinity” and “measurably inhibit,” as used herein, means a measurable change in PARP activity between a sample comprising a compound of the present invention, or composition thereof, and PARP, and an equivalent sample comprising PARP, in the absence of said compound, or composition thereof.

As used herein, the term “independently” means independently for each occurrence.

### Description of Exemplary Embodiments

As described above, in some embodiments, the present invention provides a compound of formula I:

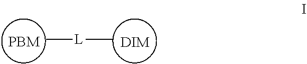

or a pharmaceutically acceptable salt thereof, wherein:
PBM is a protein targeting moiety capable of binding PARP;
L is a bivalent moiety that connects PBM to DIM; and
DIM is a deubiquitination inducing moiety, such as a deubiquitinase binding moiety (DBM).

### Protein Binding Moiety (PBM)

As defined herein and described above, PBM is a protein binding moiety capable of binding PARP. In some embodiments, PBM binds to PARP protein which then undergoes deubiquitination thereby deactivating the PARP protein.

As defined herein and described below, wherein a formula is depicted using square brackets, e.g.,

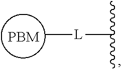

is attached to a modifiable carbon, oxygen, nitrogen or sulfur atom within PBM including substitution or replacement of a defined group in PBM.

In some embodiments, the present invention provides a compound of formula I, wherein PBM is a PARP binding moiety thereby forming a compound of formula I-a:

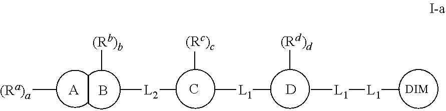

or a pharmaceutically acceptable salt thereof, wherein L and DIM are as defined above and described herein, and wherein:
Ring A and Ring B are independently fused rings selected from benzo, a 4-7 membered saturated or partially unsaturated carbocyclyl, a 4-7 membered saturated or partially unsaturated heterocyclyl with 1-3 heteroatoms independently selected from nitrogen, oxygen, and sulfur, and a 5-6 membered heteroaryl with 1-4 heteroatoms independently selected from nitrogen, oxygen and sulfur;
Ring C is a ring selected from phenylenyl, a 4-7 membered saturated or partially unsaturated carbocyclylenyl, a 4-7 membered saturated or partially unsaturated heterocyclylenyl with 1-3 heteroatoms independently selected from nitrogen, oxygen, and sulfur, and a 5-6 membered heteroarylenyl with 1-4 heteroatoms independently selected from nitrogen, oxygen and sulfur;
Ring D is a ring selected from phenylenyl, a 4-7 membered saturated or partially unsaturated carbocyclylenyl, a 4-7 membered saturated or partially unsaturated heterocyclylenyl with 1-3 heteroatoms independently selected from nitrogen, oxygen, and sulfur, and a 5-6 membered heteroarylenyl with 1-4 heteroatoms independently selected from nitrogen, oxygen and sulfur;
L¹, L² and L^{A} are independently a covalent bond or a bivalent, saturated or partially unsaturated, straight or branched C₁₋₅ hydrocarbon chain, wherein 0-3 methylene units of L¹, L² or L^{A} are independently replaced by —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, or —OC(O)NR—, —NRC(O)O—.
each R^{a}, R^{b}, R^{c}, and R^{d} is independently selected from hydrogen, R^{A}, halogen, —CN, —NO₂, —OR, —SR, —NR₂, —S(O)₂R, —S(O)₂NR₂, —S(O)R, —C(O)R, —C(O)OR, —C(O)NR₂, —C(O)NROR, —OC(O)R, —OC(O)NR₂, —NRC(O)OR, —NRC(O)R, —NRC(O)N(R)₂, and —NRS(O)₂R;
each R^{A} is independently an optionally substituted group selected from C₁₋₆ aliphatic, phenyl, a 3-12 membered saturated or partially unsaturated monocyclic, bicyclic, bridged bicyclic or spirocyclic carbocyclyl or heterocyclyl with 1-3 heteroatoms independently selected from nitrogen, oxygen, and sulfur, and a 5-6 membered heteroaryl ring having 1-4 heteroatoms independently selected from nitrogen, oxygen, and sulfur;
each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same nitrogen are optionally taken together with their intervening atoms to form a 4-7 membered saturated, partially unsaturated, or heteroaryl ring having 0-3 heteroatoms, in addition to the nitrogen atom to which they are attached, independently selected from nitrogen, oxygen, and sulfur; and
a, b, c, and d are independently 0, 1, 2, 3, or 4.

In some embodiments, the present invention provides a compound of formula I-a, wherein PBM is a PARP binding moiety thereby forming a compound of formula I-b:

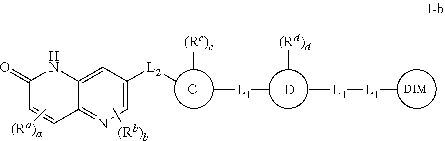

or a pharmaceutically acceptable salt thereof, wherein L and DIM are as defined above and described herein, and wherein:
Ring C is a ring selected from phenylenyl, a 4-7 membered saturated or partially unsaturated carbocyclylenyl, a 4-7 membered saturated or partially unsaturated heterocyclylenyl with 1-3 heteroatoms independently selected from nitrogen, oxygen, and sulfur, and a 5-6 membered heteroarylenyl with 1-4 heteroatoms independently selected from nitrogen, oxygen and sulfur;
Ring D is a ring selected from phenylenyl, a 4-7 membered saturated or partially unsaturated carbocyclylenyl, a 4-7 membered saturated or partially unsaturated heterocyclylenyl with 1-3 heteroatoms independently selected from nitrogen, oxygen, and sulfur, and a 5-6 membered heteroarylenyl with 1-4 heteroatoms independently selected from nitrogen, oxygen and sulfur;
L¹, L², and L^{A} are independently a covalent bond or a bivalent, saturated or partially unsaturated, straight or branched C₁₋₅ hydrocarbon chain, wherein 0-3 methylene units of L¹, L² or L^{A} are independently replaced by —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, or —OC(O)NR—, or —NRC(O)O—;
each R^{a}, R^{b}, R^{c}, and R^{d} is independently selected from hydrogen, R^{A}, halogen, —CN, —NO₂, —OR, —SR, —NR₂, —S(O)₂R, —S(O)₂NR₂, —S(O)R, —C(O)R, —C(O)OR, —C(O)NR₂, —C(O)NROR, —OC(O)R, —OC(O)NR₂, —NRC(O)OR, —NRC(O)R, —NRC(O)N(R)₂, and —NRS(O)₂R;
each R^{A} is independently an optionally substituted group selected from C₁₋₆ aliphatic, phenyl, a 3-12 membered saturated or partially unsaturated monocyclic, bicyclic, bridged bicyclic or spirocyclic carbocyclyl or heterocyclyl with 1-3 heteroatoms independently selected from nitrogen, oxygen, and sulfur, and a 5-6 membered heteroaryl ring having 1-4 heteroatoms independently selected from nitrogen, oxygen, and sulfur;
each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same nitrogen are optionally taken together with their intervening atoms to form a 4-7 membered saturated, partially unsaturated, or heteroaryl ring having 0-3 heteroatoms, in addition to the nitrogen atom to which they are attached, independently selected from nitrogen, oxygen, and sulfur; and
a, b, c, and d are independently 0, 1, 2, 3, or 4.

In some embodiments, the present invention provides a compound of formula I-b, wherein PBM is a PARP binding moiety thereby forming a compound of formula I-b-1:

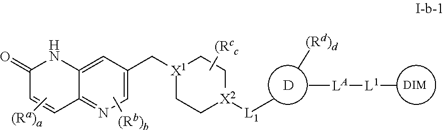

or a or a pharmaceutically acceptable salt thereof, wherein each of ring D, L¹, L^{A}, R^{a}, R^{b}, R^{c}, R^{d}, a, b, c, d, L, and DIM is as defined above and described in embodiments herein, both singly and in combination, wherein X¹ and X² are independently carbon or nitrogen.

In some embodiments, the present invention provides a compound of formula I-b, wherein PBM is a PARP binding moiety thereby forming a compound of formula I-b-2:

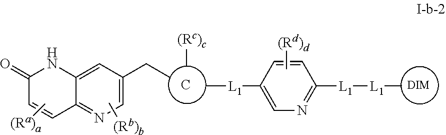

or a or a pharmaceutically acceptable salt thereof, wherein each of ring C, L¹, L^{A}, R^{a}, R^{b}, R^{c}, R^{d}, a, b, c, d, L, and DIM is as defined above and described in embodiments herein, both singly and in combination.

In some embodiments, the present invention provides a compound of formula I-a, wherein PBM is a PARP binding moiety thereby forming a compound of formula I-c:

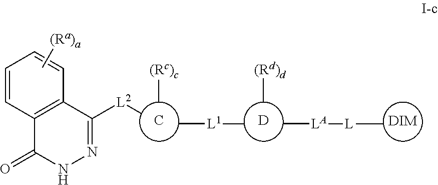

or a pharmaceutically acceptable salt thereof, wherein L and DIM are as defined above and described herein, and wherein:
Ring C is a ring selected from phenylenyl, a 4-7 membered saturated or partially unsaturated carbocyclylenyl, a 4-7 membered saturated or partially unsaturated heterocyclylenyl with 1-3 heteroatoms independently selected from nitrogen, oxygen, and sulfur, and a 5-6 membered heteroarylenyl with 1-4 heteroatoms independently selected from nitrogen, oxygen and sulfur;
Ring D is a ring selected from phenylenyl, a 4-7 membered saturated or partially unsaturated carbocyclylenyl, a 4-7 membered saturated or partially unsaturated heterocyclylenyl with 1-3 heteroatoms independently selected from nitrogen, oxygen, and sulfur, and a 5-6 membered heteroarylenyl with 1-4 heteroatoms independently selected from nitrogen, oxygen and sulfur;
L¹, L² and L^{A} are independently a covalent bond or a bivalent, saturated or partially unsaturated, straight or branched C₁₋₅ hydrocarbon chain, wherein 0-3 methylene units of L¹, L² or L^{A} are independently replaced by —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, or —OC(O)NR—, —NRC(O)O—.
each R^{a}, R^{b}, R^{c}, and R^{d} is independently selected from hydrogen, R^{A}, halogen, —CN, —NO₂, —OR, —SR, —NR₂, —S(O)₂R, —S(O)₂NR₂, —S(O)R, —C(O)R, —C(O)OR, —C(O)NR₂, —C(O)NROR, —OC(O)R, —OC(O)NR₂, —NRC(O)OR, —NRC(O)R, —NRC(O)N(R)₂, and —NRS(O)₂R;
each R^{A} is independently an optionally substituted group selected from C₁₋₆ aliphatic, phenyl, a 3-12 membered saturated or partially unsaturated monocyclic, bicyclic, bridged bicyclic or spirocyclic carbocyclyl or heterocyclyl with 1-3 heteroatoms independently selected from nitrogen, oxygen, and sulfur, and a 5-6 membered heteroaryl ring having 1-4 heteroatoms independently selected from nitrogen, oxygen, and sulfur;
each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same nitrogen are optionally taken together with their intervening atoms to form a 4-7 membered saturated, partially unsaturated, or heteroaryl ring having 0-3 heteroatoms, in addition to the nitrogen atom to which they are attached, independently selected from nitrogen, oxygen, and sulfur; and
a, b, c, and d are independently 0, 1, 2, 3, or 4.

In some embodiments, the present invention provides a compound of formula I-c, wherein PBM is a PARP binding moiety thereby forming a compound of formula I-c-1:

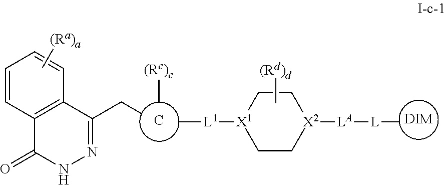

or a pharmaceutically acceptable salt thereof, wherein each of ring C, L¹, L^{A}, R^{a}, R^{c}, R^{d}, a, c, d, L, and DIM is as defined above and described in embodiments herein, both singly and in combination, wherein X¹ and X² are independently carbon or nitrogen.

In some embodiments, the present invention provides a compound of formula I-c, wherein PBM is a PARP binding moiety thereby forming a compound of formula I-c-2:

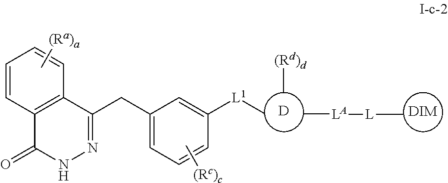

or a pharmaceutically acceptable salt thereof, wherein each of ring D, L¹, L^{A}, R^{a}, R^{c}, R^{d}, a, c, d, L, and DIM is as defined above and described in embodiments herein, both singly and in combination.

In some embodiments, a provided compound or pharmaceutically acceptable salt thereof, is selected from those wherein PBM is A-966492, AG-14361, 5-AIQ, Amelparib, 4-Aminonaphthalimide, AMXI-5001, Atamparib (RBN-2397), AZ3391, AZ6102, AZ9482, AZD2461, AZD-9574, Basroparib, Benzamide, Berberine chloride (NSC 646666), BGP-15 2HCl, BYK204165, CEP-8983, CEP-9722, Dehydrocorydaline, DPQ, DR2313, E7016 (GPI 21016), EB-47 dihydrochloride, Fluzoparib (SHR3162), G007-LK, GeA-69, HI-TOPK-032, 4-Hydroxyquinazoline, Iniparib (BSI-201), INO-1001 (3-Aminobenzamide), 1,5-Isoquinolinediol, KCL-440, KSQ-4279, Licochalcone D, M2912 (MSC2504877), ME0328, 3-Methoxybenzamide, 2-Methylquinazolin-4-ol, MN 64, Nesuparib (JPI-547), Niraparib (ZL-2306), Niraparib tosylate (MK-4827), NMS-03305293, NMS-P118, NU1025, NVP-TNKS656, Olaparib (AZD2281; KU0059436), Pamiparib (BGB-290), PARP11 inhibitor ITK7, PARP-1-IN-1, PARP1-IN-6, PARP1-IN-7, PARP-2-IN-2, PARP7-IN-14, Picolinamide, PJ34 HCl, RBN012759, RK-287107, RP12146, Rucaparib (PF-01367338), Saruparib (AZD5305), Senaparib (IMP4297), Stenoparib (E7449; 2X-121), Talazoparib (BMN-673), Tankyrase-IN-2, 4′,5,7-Trimethoxyflavone, UPF 1069, Veliparib (ABT-888), Venadaparib (IDX-1197), or WIKI4.

As defined above and described herein, Ring A and Ring B are independently fused rings selected from benzo, a 4-7 membered saturated or partially unsaturated carbocyclyl, a 4-7 membered saturated or partially unsaturated heterocyclyl with 1-3 heteroatoms independently selected from nitrogen, oxygen, and sulfur, and a 5-6 membered heteroaryl with 1-4 heteroatoms independently selected from nitrogen, oxygen and sulfur.

In some embodiments, Ring A is a fused benzo ring. In some embodiments, Ring A is a 4-7 membered saturated or partially unsaturated carbocyclyl. In some embodiments, Ring A is a 4-7 membered saturated or partially unsaturated heterocyclyl with 1-3 heteroatoms independently selected from nitrogen, oxygen, and sulfur. In some embodiments, Ring A is a 5-6 membered heteroaryl with 1-4 heteroatoms independently selected from nitrogen, oxygen and sulfur.

In some embodiments, ring A is selected from those depicted in the compounds of Table 1 below.

In some embodiments, Ring B is a fused benzo ring. In some embodiments, Ring B is a 4-7 membered saturated or partially unsaturated carbocyclyl. In some embodiments, Ring B is a 4-7 membered saturated or partially unsaturated heterocyclyl with 1-3 heteroatoms independently selected from nitrogen, oxygen, and sulfur. In some embodiments, Ring B is a 5-6 membered heteroaryl with 1-4 heteroatoms independently selected from nitrogen, oxygen and sulfur.

In some embodiments, ring B is selected from those depicted in the compounds of Table 1 below.

As defined above and described herein, Ring C is a ring selected from phenylenyl, a 4-7 membered saturated or partially unsaturated carbocyclylenyl, a 8-10 membered saturated or partially unsaturated bridged bicyclic carbocyclylenyl, a 4-7 membered saturated or partially unsaturated heterocyclylenyl with 1-3 heteroatoms independently selected from nitrogen, oxygen, and sulfur, a 8-10 membered saturated or partially unsaturated bridged bicyclic heterocyclylenyl with 1-3 heteroatoms independently selected from nitrogen, oxygen, and sulfur, and a 5-6 membered heteroarylenyl with 1-4 heteroatoms independently selected from nitrogen, oxygen and sulfur.

In some embodiments, ring C is a phenylenyl. In some embodiments, ring C is a 4-7 membered saturated or partially unsaturated carbocyclylenyl. In some embodiments, ring C is a 8-10 membered saturated or partially unsaturated bridged bicyclic carbocyclylenyl. In some embodiments, ring C is a 4-7 membered saturated or partially unsaturated heterocyclylenyl with 1-3 heteroatoms independently selected from nitrogen, oxygen, and sulfur. In some embodiments, ring C is a 8-10 membered saturated or partially unsaturated bridged bicyclic heterocyclylenyl with 1-3 heteroatoms independently selected from nitrogen, oxygen, and sulfur. In some embodiments, ring C is a 5-6 membered heteroarylenyl with 1-4 heteroatoms independently selected from nitrogen, oxygen and sulfur.

In some embodiments, ring C is selected from those depicted in the compounds of Table 1 below.

As defined above and described herein, ring D is a ring selected from phenylenyl, a 4-7 membered saturated or partially unsaturated carbocyclylenyl, a 4-7 membered saturated or partially unsaturated heterocyclylenyl with 1-3 heteroatoms independently selected from nitrogen, oxygen, and sulfur, and a 5-6 membered heteroarylenyl with 1-4 heteroatoms independently selected from nitrogen, oxygen and sulfur.

In some embodiments, ring D is a phenylenyl. In some embodiments, ring D is a 4-7 membered saturated or partially unsaturated carbocyclylenyl. In some embodiments, ring D is a 4-7 membered saturated or partially unsaturated heterocyclylenyl with 1-3 heteroatoms independently selected from nitrogen, oxygen, and sulfur. In some embodiments, ring D is and a 5-6 membered heteroarylenyl with 1-4 heteroatoms independently selected from nitrogen, oxygen and sulfur.

In some embodiments, ring D is

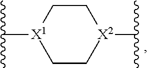

wherein X¹ and X² are independently carbon or nitrogen. In some embodiments, ring D is

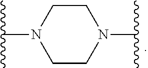

In some embodiments, ring D is selected from those depicted in the compounds of Table 1 below.

As defined above and described herein, X¹ and X² are independently nitrogen or carbon.

In some embodiments, X¹ and X² are independently nitrogen or carbon. In some embodiments, at least one of X¹ and X² is nitrogen. In some embodiments, X¹ is nitrogen and X² is carbon. In some embodiments, X¹ is carbon and X² is nitrogen. In some embodiments, X¹ and X² are nitrogen.

In some embodiments, X¹ is selected from those depicted in the compounds of Table 1 below.

In some embodiments, X² is selected from those depicted in the compounds of Table 1 below.

As defined above and described herein, ring W is a ring selected from phenylenyl, a 4-7 membered saturated or partially unsaturated carbocyclylenyl, a 4-7 membered saturated or partially unsaturated heterocyclylenyl with 1-3 heteroatoms independently selected from nitrogen, oxygen, and sulfur, and a 5-6 membered heteroarylenyl with 1-4 heteroatoms independently selected from nitrogen, oxygen and sulfur.

As defined above and described herein, L¹, is a covalent bond or a bivalent, saturated or partially unsaturated, straight or branched C₁₋₅ hydrocarbon chain, wherein 0-3 methylene units of L¹, L² or L^{A} are independently replaced by —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, or —OC(O)NR—, —NRC(O)O—.

In some embodiments, L¹ is a covalent bond. In some embodiments, L¹ is a bivalent, saturated or partially unsaturated, straight or branched C₁₋₅ hydrocarbon chain, wherein 0-3 methylene units of L¹ are independently replaced by —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, or —OC(O)NR—, —NRC(O)O—.

In some embodiments, L¹ is —O—. In some embodiments, L¹ is —NR—. In some embodiments, L¹ is —S—. In some embodiments, L¹ is —OC(O)—. In some embodiments, L¹ is —C(O)O—. In some embodiments, L¹ is —C(O)—. In some embodiments, L¹ is —S(O)—. In some embodiments, L¹ is —S(O)₂—. In some embodiments, L¹ is —NRS(O)₂—. In some embodiments, L¹ is —S(O)₂NR—. In some embodiments, L¹ is —NRC(O)—. In some embodiments, L¹ is —C(O)NR—. In some embodiments, L¹ is or —OC(O)NR—. In some embodiments, L¹ is —NRC(O)O—.

In some embodiments, L¹ is selected from those depicted in the compounds of Table 1 below.

As defined above and described herein, L^{A}, is a covalent bond or a bivalent, saturated or partially unsaturated, straight or branched C₁₋₅ hydrocarbon chain, wherein 0-3 methylene units of L^{A} are independently replaced by —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, or —OC(O)NR—, —NRC(O)O—.

In some embodiments, L^{A} is a covalent bond. In some embodiments, L^{A} is a bivalent, saturated or partially unsaturated, straight or branched C₁₋₅ hydrocarbon chain, wherein 0-3 methylene units of L^{A} are independently replaced by —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, or —OC(O)NR—, —NRC(O)O—.

In some embodiments, L^{A} is —O—. In some embodiments, L^{A} is —NR—. In some embodiments, L^{A} is —S—. In some embodiments, L^{A} is —OC(O)—. In some embodiments, L^{A} is —C(O)O—. In some embodiments, L^{A} is —C(O)—. In some embodiments, L^{A} is —S(O)—. In some embodiments, L^{A} is —S(O)₂—. In some embodiments, L^{A} is —NRS(O)₃—. In some embodiments, L^{A} is —S(O)₂NR—. In some embodiments, L^{A} is —NRC(O)—. In some embodiments, L^{A} is —C(O)NR—. In some embodiments, L^{A} is or —OC(O)NR—. In some embodiments, L^{A} is —NRC(O)O—.

In some embodiments, L^{A} is selected from those depicted in the compounds of Table 1 below.

As defined above and described herein, each R^{a} is independently selected from hydrogen, R^{A}, halogen, —CN, —NO₂, —OR, —SR, —NR₂, —S(O)₂R, —S(O)₂NR₂, —S(O)R, —C(O)R, —C(O)OR, —C(O)NR₂, —C(O)NROR, —OC(O)R, —OC(O)NR₂, —NRC(O)OR, —NRC(O)R, —NRC(O)N(R)₂, and —NRS(O)₂R.

In some embodiments, one or more of R is hydrogen. In some embodiments, one or more of R^{a} is R^{A}. In some embodiments, one or more of R is halogen. In some embodiments, one or more of R is —CN. In some embodiments, one or more of R is —NO₂. In some embodiments, one or more of R is —OR. In some embodiments, one or more of R is —SR. In some embodiments, one or more of R is —NR₂. In some embodiments, one or more of R is —S(O)₂R. In some embodiments, one or more of R is —S(O)₂NR₂. In some embodiments, one or more of R is —S(O)R. In some embodiments, one or more of R is —C(O)R. In some embodiments, one or more of R^{a} is —C(O)OR. In some embodiments, one or more of R^{a} is —C(O)NR₂. In some embodiments, one or more of R is —C(O)NROR. In some embodiments, one or more of R^{a} is —OC(O)R. In some embodiments, one or more of R^{a} is —OC(O)NR₂. In some embodiments, one or more of R is —NRC(O)OR. In some embodiments, one or more of R is —NRC(O)R. In some embodiments, one or more of R^{a} is —NRC(O)N(R)₂. In some embodiments, one or more of R^{a} is —NRS(O)₂R.

In some embodiments, one or more of R is methyl. In some embodiments, one or more of R is ethyl.

In some embodiments, R is selected from those depicted in the compounds of Table 1 below.

As defined above and described herein, each R^{b} is independently selected from hydrogen, R^{A}, halogen, —CN, —NO₂, —OR, —SR, —NR₂, —S(O)₂R, —S(O)₂NR₂, —S(O)R, —C(O)R, —C(O)OR, —C(O)NR₂, —C(O)NROR, —OC(O)R, —OC(O)NR₂, —NRC(O)OR, —NRC(O)R, —NRC(O)N(R)₂, and —NRS(O)₂R.

In some embodiments, one or more of R is hydrogen. In some embodiments, one or more of R^{b} is R^{A}. In some embodiments, one or more of R is halogen. In some embodiments, one or more of R is —CN. In some embodiments, one or more of R is —NO₂. In some embodiments, one or more of R is —OR. In some embodiments, one or more of R is —SR. In some embodiments, one or more of R is —NR₂. In some embodiments, one or more of R is —S(O)₂R. In some embodiments, one or more of R is —S(O)₂NR₂. In some embodiments, one or more of R is —S(O)R. In some embodiments, one or more of R is —C(O)R. In some embodiments, one or more of R^{b} is —C(O)OR. In some embodiments, one or more of R^{b} is —C(O)NR₂. In some embodiments, one or more of R is —C(O)NROR. In some embodiments, one or more of R is —OC(O)R. In some embodiments, one or more of R is —OC(O)NR₂. In some embodiments, one or more of R is —NRC(O)OR. In some embodiments, one or more of R is —NRC(O)R. In some embodiments, one or more of R is —NRC(O)N(R)₂. In some embodiments, one or more of R is —NRS(O)₂R.

In some embodiments, R is selected from those depicted in the compounds of Table 1 below.

As defined above and described herein, each R^{c} is independently selected from hydrogen, R^{A}, halogen, —CN, —NO₂, —OR, —SR, —NR₂, —S(O)₂R, —S(O)₂NR₂, —S(O)R, —C(O)R, —C(O)OR, —C(O)NR₂, —C(O)NROR, —OC(O)R, —OC(O)NR₂, —NRC(O)OR, —NRC(O)R, —NRC(O)N(R)₂, and —NRS(O)₂R.

In some embodiments, one or more of R is hydrogen. In some embodiments, one or more of R^{c} is R^{A}. In some embodiments, one or more of R is halogen. In some embodiments, one or more of R is —CN. In some embodiments, one or more of R is —NO₂. In some embodiments, one or more of R is —OR. In some embodiments, one or more of R is —SR. In some embodiments, one or more of R is —NR₂. In some embodiments, one or more of R is —S(O)₂R. In some embodiments, one or more of R is —S(O)₂NR₂. In some embodiments, one or more of R is —S(O)R. In some embodiments, one or more of R is —C(O)R. In some embodiments, one or more of R^{c} is —C(O)OR. In some embodiments, one or more of R^{c} is —C(O)NR₂. In some embodiments, one or more of R is —C(O)NROR. In some embodiments, one or more of R is —OC(O)R. In some embodiments, one or more of R is —OC(O)NR₂. In some embodiments, one or more of R is —NRC(O)OR. In some embodiments, one or more of R is —NRC(O)R. In some embodiments, one or more of R is —NRC(O)N(R)₂. In some embodiments, one or more of R is —NRS(O)₂R.

In some embodiments, one or more of R is fluoro.

In some embodiments, R is selected from those depicted in the compounds of Table 1 below.

As defined above and described herein, each R^{d} is independently selected from hydrogen, R^{A}, halogen, —CN, —NO₂, —OR, —SR, —NR₂, —S(O)₂R, —S(O)₂NR₂, —S(O)R, —C(O)R, —C(O)OR, —C(O)NR₂, —C(O)NROR, —OC(O)R, —OC(O)NR₂, —NRC(O)OR, —NRC(O)R, —NRC(O)N(R)₂, and —NRS(O)₂R.

In some embodiments, one or more of R^{d} is hydrogen. In some embodiments, one or more of R^{d} is R^{A}. In some embodiments, one or more of R^{d} is halogen. In some embodiments, one or more of R^{d} is —CN. In some embodiments, one or more of R^{d} is —NO₂. In some embodiments, one or more of R^{d} is —OR. In some embodiments, one or more of R^{d} is —SR. In some embodiments, one or more of R^{d} is —NR₂. In some embodiments, one or more of R^{d} is —S(O)₂R. In some embodiments, one or more of R^{d} is —S(O)₂NR₂. In some embodiments, one or more of R^{d} is —S(O)R. In some embodiments, one or more of R^{d} is —C(O)R. In some embodiments, one or more of R^{d} is —C(O)OR. In some embodiments, one or more of R^{d} is —C(O)NR₂. In some embodiments, one or more of R^{d} is —C(O)NROR. In some embodiments, one or more of R^{d} is —OC(O)R. In some embodiments, one or more of R^{d} is —OC(O)NR₂. In some embodiments, one or more of R^{d} is —NRC(O)OR. In some embodiments, one or more of R^{d} is —NRC(O)R. In some embodiments, one or more of R^{d} is —NRC(O)N(R)₂. In some embodiments, one or more of R^{d} is —NRS(O)₂R.

In some embodiments, R^{d} is selected from those depicted in the compounds of Table 1 below.

As defined above and described herein, each R^{A} is independently an optionally substituted group selected from C₁₋₆ aliphatic, phenyl, a 3-12 membered saturated or partially unsaturated monocyclic, bicyclic, bridged bicyclic or spirocyclic carbocyclyl or heterocyclyl with 1-3 heteroatoms independently selected from nitrogen, oxygen, and sulfur, and a 5-6 membered heteroaryl ring having 1-4 heteroatoms independently selected from nitrogen, oxygen, and sulfur.

In some embodiments, of R^{A} is an optionally substituted C₁₋₆ aliphatic. In some embodiments, R^{A} is a phenyl. In some embodiments, R^{A} is a 3-12 membered saturated or partially unsaturated monocyclic. In some embodiments, R^{A} is an optionally substituted bicyclic. In some embodiments, of R^{A} is an optionally substituted bridged bicyclic or spirocyclic carbocyclyl. In some embodiments, R^{A} is a heterocyclyl with 1-3 heteroatoms independently selected from nitrogen, oxygen, and sulfur. In some embodiments, R^{A} is an optionally substituted 5-6 membered heteroaryl ring having 1-4 heteroatoms independently selected from nitrogen, oxygen, and sulfur.

In some embodiments, R is selected from those depicted in the compounds of Table 1 below.

As defined above and described herein, each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or two R groups on the same nitrogen are optionally taken together with their intervening atoms to form a 4-7 membered saturated, partially unsaturated, or heteroaryl ring having 0-3 heteroatoms, in addition to the nitrogen atom to which they are attached, independently selected from nitrogen, oxygen, and sulfur.

In some embodiments, one or more of R is hydrogen. In some embodiments, one or more of R is a C₁₋₆ aliphatic. In some embodiments, one or more of R is phenyl. In some embodiments, one or more of R is a 4-7 membered saturated or partially unsaturated heterocyclic having 1-2 heteroatoms independently selected from nitrogen, oxygen and sulfur. In some embodiments, one or more of R is and a 5-6 membered heteroaryl ring having 1-4 heteroatoms independently selected from nitrogen, oxygen, and sulfur. In some embodiments, two R groups on the same nitrogen are optionally taken together with their intervening atoms to form a 4-7 membered saturated, partially unsaturated, or heteroaryl ring having 0-3 heteroatoms, in addition to the nitrogen atom to which they are attached, independently selected from nitrogen, oxygen, and sulfur.

In some embodiments, R is selected from those depicted in the compounds of Table 1 below.

As defined above and described herein, a is 0, 1, 2, 3, or 4. In some embodiments, a is 0. In some embodiments, a is 1. In some embodiments, a is 2. In some embodiments, a is 3. In some embodiments, a is 4.

In some embodiments, a is selected from those depicted in the compounds of Table 1 below.

As defined above and described herein, b is 0, 1, 2, 3, or 4. In some embodiments, b is 0. In some embodiments, b is 1. In some embodiments, b is 2. In some embodiments, b is 3. In some embodiments, b is 4.

In some embodiments, b is selected from those depicted in the compounds of Table 1 below.

As defined above and described herein, c is 0, 1, 2, 3, or 4. In some embodiments, c is 0. In some embodiments, c is 1. In some embodiments, c is 2. In some embodiments, c is 3. In some embodiments, c is 4.

In some embodiments, c is selected from those depicted in the compounds of Table 1 below.

As defined above and described herein, d is 0, 1, 2, 3, or 4. In some embodiments, d is 0. In some embodiments, d is 1. In some embodiments, d is 2. In some embodiments, d is 3. In some embodiments, d is 4.

In some embodiments, d is selected from those depicted in the compounds of Table 1 below.

In some embodiments, PBM is

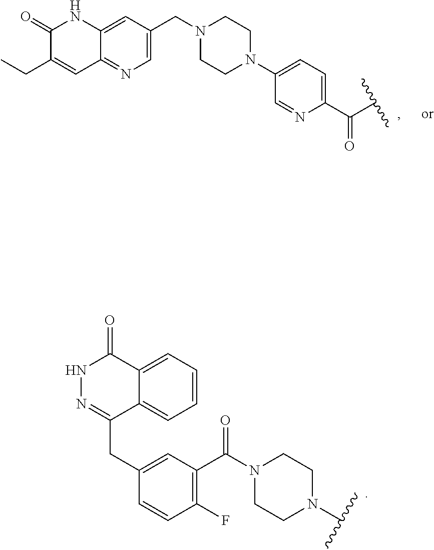 [SMILES: *C(=O)c1ccc(N2CCN(Cc3cnc4cc(CC)c(=O)[nH]c4c3)CC2)cn1 ; *N1CCN(C(=O)c2cc(Cc3n[nH]c(=O)c4ccccc34)ccc2F)CC1]

In some embodiments, PBM is

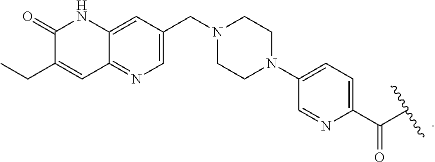 [SMILES: *C(=O)c1ccc(N2CCN(Cc3cnc4cc(CC)c(=O)[nH]c4c3)CC2)cn1]

In some embodiments, PBM is

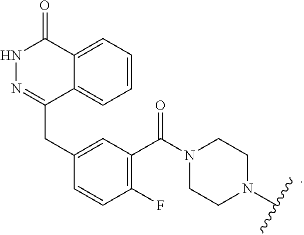 [SMILES: *N1CCN(C(=O)c2cc(Cc3n[nH]c(=O)c4ccccc34)ccc2F)CC1]

In some embodiments, PBM is selected from those depicted in Table 1, below.

In some embodiments, PBM is selected from those depicted in paragraph [0057], above.

### Deubiquitinase Binding Moiety (DBM)

In some embodiments, DIM is DBM. In some embodiments, DIM is a DUB ligand well known to one of ordinary skill in the art including those described in Caba et al., Biomolecules. 2022, 12(5):703; Harrigan et al., Nat Rev Drug Discov. 2018, 17(1), 57-78; Lange et al., Mol Cell. 2022, 82(1), 15-29; Pinto-Fernindez et al., Front Chem. 2019, 7, 592; Schauer et al., J Med Chem. 2020, 63(6), 2731-2750; and Wertz et al., Cell Chem Biol. 2019, 26(2), 156-177, the entirety of each of which is hereby incorporated herein by reference.

In some embodiments, the DBM binds to OTUD1, OTUD7B, OTUB1, USP2, USP5, USP7, USP21, USP28, USP7, USP10, USP15, and/or AMSH. In some embodiments, the DBM binds to OTUD1.

In some embodiments, the DBM binds to OTUD7B. In some embodiments, the DBM binds to OTUB1. In some embodiments, the DBM binds to USP2. In some embodiments, the DBM binds to USP5. In some embodiments, the DBM binds to USP7. In some embodiments, the DBM binds to USP21. In some embodiments, the DBM binds to USP28. In some embodiments, the DBM binds to USP7. In some embodiments, the DBM binds to USP10. In some embodiments, the DBM binds to USP15. In some embodiments, the DBM binds to AMSH.

As defined herein and described below, wherein a formula is depicted using square brackets, e.g.,

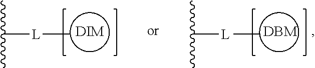

L is attached to a modifiable carbon, oxygen, or nitrogen atom within DIM or DBM including substitution or replacement of a defined group in DIM or DBM.

In some embodiments, the present invention provides a compound of formula I:

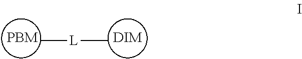

or a pharmaceutically acceptable salt thereof, wherein L and PBM are as described above and herein, and DIM is a deubiquitination inducing moiety, such as a deubiquitinase binding moiety (DBM).

In some embodiments, DIM is a DBM as described above and herein.

In some embodiments, the present invention provides a compound of formula I, wherein DBM is a deubiquitinase binding moiety thereby forming a compound of formula I-aa:

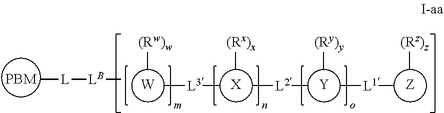

or a pharmaceutically acceptable salt thereof, wherein L and PBM are as defined above and described herein, and wherein:
Ring Z is a ring selected from phenyl, a 4-7 membered saturated or partially unsaturated carbocyclic ring, an 8-10 membered bicyclic aromatic carbocyclic ring, a 4-8 membered saturated or partially unsaturated monocyclic heterocyclic ring having 1-2 heteroatoms independently selected from nitrogen, oxygen, or sulfur, a 5-6 membered monocyclic heteroaromatic ring having 1-4 heteroatoms independently selected from nitrogen, oxygen, or sulfur, and an 8-10 membered bicyclic heteroaromatic ring having 1-5 heteroatoms independently selected from nitrogen, oxygen, or sulfur;
Ring Y is a ring selected from a phenylenyl, 4-7 membered saturated or partially unsaturated carbocyclylenyl, an 8-10 membered bicyclic aromatic carbocyclylenyl, a 4-8 membered saturated or partially unsaturated monocyclic heterocyclylenyl having 1-2 heteroatoms independently selected from nitrogen, oxygen, or sulfur, a 5-6 membered monocyclic heteroarylenyl having 1-4 heteroatoms independently selected from nitrogen, oxygen, or sulfur, and an 8-10 membered bicyclic heteroarylenyl having 1-5 heteroatoms independently selected from nitrogen, oxygen, or sulfur;
Ring X is a ring selected from phenylenyl, a 4-7 membered saturated or partially unsaturated carbocyclylenyl, a 4-7 membered saturated or partially unsaturated heterocyclylenyl with 1-3 heteroatoms independently selected from nitrogen, oxygen, and sulfur, and a 5-6 membered heteroarylenyl with 1-4 heteroatoms independently selected from nitrogen, oxygen and sulfur;
Ring W is a ring selected from phenylenyl, a 4-7 membered saturated or partially unsaturated carbocyclylenyl, a 4-7 membered saturated or partially unsaturated heterocyclylenyl with 1-3 heteroatoms independently selected from nitrogen, oxygen, and sulfur, and a 5-6 membered heteroarylenyl with 1-4 heteroatoms independently selected from nitrogen, oxygen and sulfur;
each of L^{B}, L^{1′}, L^{2′} and L^{3′} is independently a covalent bond or a bivalent, saturated or partially unsaturated, straight or branched C₁₋₅ hydrocarbon chain, wherein 0-3 methylene units of L^{B}, L^{1′}, L^{2′} or L are independently replaced by —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, or —OC(O)NR—, —NRC(O)O—;
each R^{w}, R^{x}, R^{y}, and R^{z} is independently selected from hydrogen, R^{B}, halogen, —CN, —NO₂, —OR, —SR, —NR₂, —S(O)₂R, —S(O)₂NR₂, —S(O)R, —C(O)R, —C(O)OR, —C(O)NR₂, —C(O)NROR, —OC(O)R, —OC(O)NR₂, —NRC(O)OR, —NRC(O)R, —NRC(O)N(R)₂, and —NRS(O)₂R;
each R^{B} is independently an optionally substituted group selected from C₁₋₆ aliphatic, phenyl, a 3-12 membered saturated or partially unsaturated monocyclic, bicyclic, bridged bicyclic or spirocyclic carbocyclyl or heterocyclyl with 1-3 heteroatoms independently selected from nitrogen, oxygen, and sulfur, and a 5-6 membered heteroaryl ring having 1-4 heteroatoms independently selected from nitrogen, oxygen, and sulfur;
each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same nitrogen are optionally taken together with their intervening atoms to form a 4-7 membered saturated, partially unsaturated, or heteroaryl ring having 0-3 heteroatoms, in addition to the nitrogen atom to which they are attached, independently selected from nitrogen, oxygen, and sulfur; and
m, n, and o are independently 0 or 1; and
w, x, y, and z are independently 0, 1, 2, 3, or 4.

In some embodiments, the present invention provides a compound of formula I-aa, wherein DBM is a deubiquitinase binding moiety thereby forming a compound of formula I-bb:

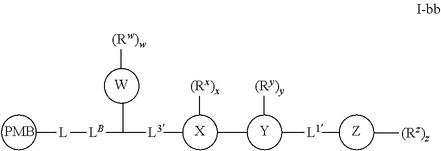

or a pharmaceutically acceptable salt thereof, wherein L and PBM are as defined above and described herein, and wherein:
Ring Z is a ring selected from phenyl, a 4-7 membered saturated or partially unsaturated carbocyclic ring, a 4-7 membered saturated or partially unsaturated heterocyclic ring with 1-3 heteroatoms independently selected from nitrogen, oxygen, and sulfur, and a 5-6 membered heteroaromatic ring with 1-4 heteroatoms independently selected from nitrogen, oxygen and sulfur;
Ring Y is a ring selected from phenylenyl, a 4-7 membered saturated or partially unsaturated carbocyclylenyl, a 4-7 membered saturated or partially unsaturated heterocyclylenyl with 1-3 heteroatoms independently selected from nitrogen, oxygen, and sulfur, and a 5-6 membered heteroarylenyl with 1-4 heteroatoms independently selected from nitrogen, oxygen and sulfur;
Ring X is a ring selected from phenylenyl, a 4-7 membered saturated or partially unsaturated carbocyclylenyl, a 4-7 membered saturated or partially unsaturated heterocyclylenyl with 1-3 heteroatoms independently selected from nitrogen, oxygen, and sulfur, and a 5-6 membered heteroarylenyl with 1-4 heteroatoms independently selected from nitrogen, oxygen and sulfur;
Ring W is a ring selected from phenylenyl, a 4-7 membered saturated or partially unsaturated carbocyclylenyl, a 4-7 membered saturated or partially unsaturated heterocyclylenyl with 1-3 heteroatoms independently selected from nitrogen, oxygen, and sulfur, and a 5-6 membered heteroarylenyl with 1-4 heteroatoms independently selected from nitrogen, oxygen and sulfur;
each L^{B}, L^{1′}, and L^{3′} is independently a covalent bond or a bivalent, saturated or partially unsaturated, straight or branched C₁₋₅ hydrocarbon chain, wherein 0-3 methylene units of L^{B}, L^{1′}, or L³ are independently replaced by —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, or —OC(O)NR—, —NRC(O)O—;
each R^{w}, R^{x}, R^{y}, and R^{z} is independently selected from hydrogen, R^{B}, halogen, —CN, —NO₂, —OR, —SR, —NR₂, —S(O)₂R, —S(O)₂NR₂, —S(O)R, —C(O)R, —C(O)OR, —C(O)NR₂, —C(O)NROR, —OC(O)R, —OC(O)NR₂, —NRC(O)OR, —NRC(O)R, —NRC(O)N(R)₂, and —NRS(O)₂R;
each R^{B} is independently an optionally substituted group selected from C₁₋₆ aliphatic, phenyl, a 3-12 membered saturated or partially unsaturated monocyclic, bicyclic, bridged bicyclic or spirocyclic carbocyclyl or heterocyclyl with 1-3 heteroatoms independently selected from nitrogen, oxygen, and sulfur, and a 5-6 membered heteroaryl ring having 1-4 heteroatoms independently selected from nitrogen, oxygen, and sulfur;
each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same nitrogen are optionally taken together with their intervening atoms to form a 4-7 membered saturated, partially unsaturated, or heteroaryl ring having 0-3 heteroatoms, in addition to the nitrogen atom to which they are attached, independently selected from nitrogen, oxygen, and sulfur; and
w, x, y, and z are independently 0, 1, 2, 3, or 4.

In some embodiments, the present invention provides a compound of formula I, wherein DBM is a deubiquitinase binding moiety thereby forming a compound of formula I-bb:

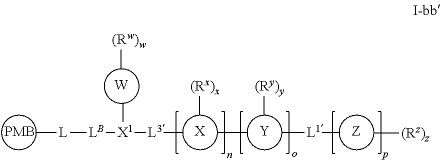

or a pharmaceutically acceptable salt thereof, wherein L and PBM are as defined above and described herein, and wherein:
Ring Z is a ring selected from phenyl, a 3-7 membered saturated or partially unsaturated monocyclic, bicyclic, bridged bicyclic or spirocyclic carbocyclic ring, a 3-7 membered saturated or partially unsaturated monocyclic, bicyclic, bridged bicyclic or spirocyclic heterocyclic ring with 1-3 heteroatoms independently selected from nitrogen, oxygen, and sulfur, and a 5-6 membered heteroaromatic ring with 1-4 heteroatoms independently selected from nitrogen, oxygen and sulfur;
Ring Y is a ring selected from phenylenyl, a 4-8 membered saturated or partially unsaturated carbocyclylenyl, a 4-8 membered saturated or partially unsaturated monocyclic, bicyclic, bridged bicyclic or spirocyclic heterocyclylenyl with 1-3 heteroatoms independently selected from nitrogen, oxygen, and sulfur, and a 5-6 membered heteroarylenyl with 1-4 heteroatoms independently selected from nitrogen, oxygen and sulfur;
Ring X is a ring selected from phenylenyl, a 4-7 membered saturated or partially unsaturated carbocyclylenyl, a 4-7 membered saturated or partially unsaturated heterocyclylenyl with 1-3 heteroatoms independently selected from nitrogen, oxygen, and sulfur, and a 5-6 membered heteroarylenyl with 1-4 heteroatoms independently selected from nitrogen, oxygen and sulfur;
Ring W is a ring selected from phenylenyl, a 4-7 membered saturated or partially unsaturated monocyclic, bicyclic, bridged bicyclic or spirocyclic carbocyclylenyl, a 4-7 membered saturated or partially unsaturated monocyclic, bicyclic, bridged bicyclic or spirocyclic heterocyclylenyl with 1-3 heteroatoms independently selected from nitrogen, oxygen, and sulfur, and a 5-6 membered heteroarylenyl with 1-4 heteroatoms independently selected from nitrogen, oxygen and sulfur;
each L^{B}, L^{1′}, and L^{3′} is independently a covalent bond or an optionally substituted bivalent, saturated or partially unsaturated, straight or branched C₁₋₅ hydrocarbon chain, wherein 0-3 methylene units of L^{B}, L^{1′}, or L^{3′} are independently replaced by —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, or —OC(O)NR—, —NRC(O)O—;
each R^{w}, R^{x}, R^{y}, and R^{z} is independently selected from hydrogen, R^{B}, halogen, —CN, —NO₂, —OR, —SR, —NR₂, —S(O)₂R, —S(O)₂NR₂, —S(O)R, —C(O)R, —C(O)OR, —C(O)NR₂, —C(O)NROR, —OC(O)R, —OC(O)NR₂, —NRC(O)OR, —NRC(O)R, —NRC(O)N(R)₂, and —NRS(O)₂R;
each R^{B} is independently an optionally substituted group selected from C₁₋₆ aliphatic, phenyl, a 3-12 membered saturated or partially unsaturated monocyclic, bicyclic, bridged bicyclic or spirocyclic carbocyclyl or heterocyclyl with 1-3 heteroatoms independently selected from nitrogen, oxygen, and sulfur, and a 5-6 membered heteroaryl ring having 1-4 heteroatoms independently selected from nitrogen, oxygen, and sulfur;
each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same nitrogen are optionally taken together with their intervening atoms to form a 4-7 membered saturated, partially unsaturated, or heteroaryl ring having 0-3 heteroatoms, in addition to the nitrogen atom to which they are attached, independently selected from nitrogen, oxygen, and sulfur;
X¹ is carbon or nitrogen;
m, n, o, and p are independently 0 or 1; and
w, x, y, and z are independently 0, 1, 2, 3, or 4.

In some embodiments, the present invention provides a compound of formula I-bb or I-bb′, wherein DBM is a deubiquitinase binding moiety thereby forming a compound of formula I-bb-1:

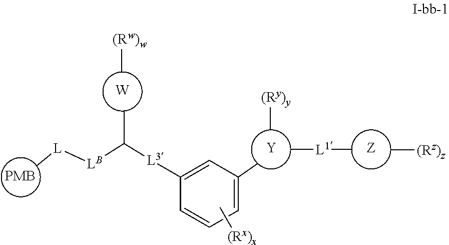

or a pharmaceutically acceptable salt thereof, wherein each of ring W, ring Y, ring Z, L^{B}, zL^{3′}, L^{1′}, R^{w}, R^{x}, R^{y}, R^{z}, w, x, y, z, L, and PBM is as defined above and described in embodiments herein, both singly and in combination.

In some embodiments, the present invention provides a compound of formula I-bb or I-bb′, wherein DBM is a deubiquitinase binding moiety thereby forming a compound of formula I-bb-2:

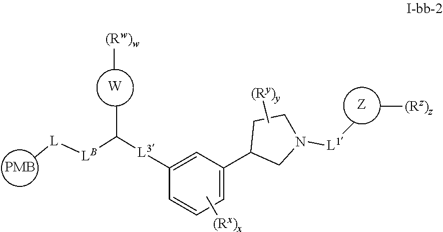

or a pharmaceutically acceptable salt thereof, wherein each of ring W, ring Z, L^{B}, L^{3′}, L^{1′}, R^{w}, R^{x}, R^{y}, R^{z}, w, x, y, z, L, and PBM is as defined above and described in embodiments herein, both singly and in combination.

In some embodiments, the present invention provides a compound of formula I-bb or I-bb′, wherein DBM is a deubiquitinase binding moiety thereby forming a compound of formula I-bb-3:

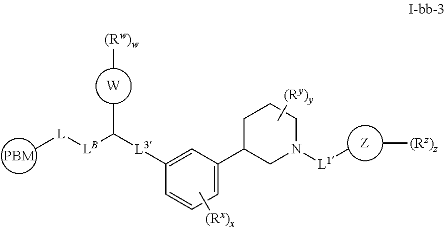

or a pharmaceutically acceptable salt thereof, wherein each of ring W, ring Z, L^{B}, L^{3′}, L^{1′}, R^{w}, R^{x}, R^{y}, R^{z}, w, x, y, z, L, and PBM is as defined above and described in embodiments herein, both singly and in combination.

In some embodiments, the present invention provides a compound of formula I-bb or I-bb′, wherein DBM is a deubiquitinase binding moiety thereby forming a compound of formula I-bb-4 or I-bb-4′:

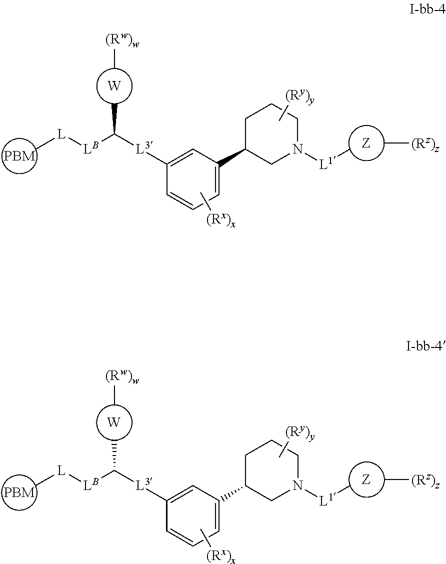

or a pharmaceutically acceptable salt thereof, wherein each of ring W, ring Z, L^{B} L³, L¹, R^{w}, R^{x}, R^{y}, R^{z}, w, x, y, z, L, and PBM is as defined above and described in embodiments herein, both singly and in combination.

In some embodiments, the present invention provides a compound of formula I-bb or I-bb′, wherein DBM is a deubiquitinase binding moiety thereby forming a compound of formula I-bb-5 or I-bb-5′:

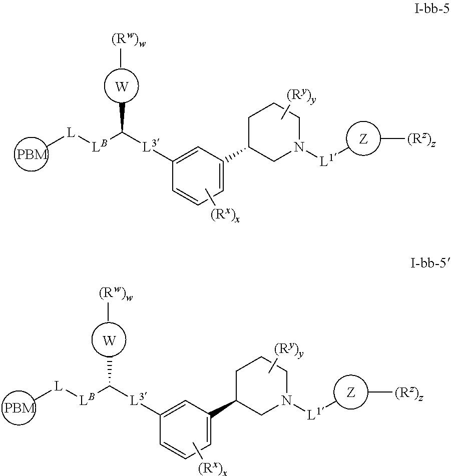

or a pharmaceutically acceptable salt thereof, wherein each of ring W, ring Z, L^{B}, L^{3′}, L^{1′}, R^{w}, R^{x}, R^{y}, R^{z}, w, x, y, z, L, and PBM is as defined above and described in embodiments herein, both singly and in combination.

In some embodiments, the present invention provides a compound of formula I-bb or I-bb′, wherein DBM is a deubiquitinase binding moiety thereby forming a compound of formula I-bb-6:

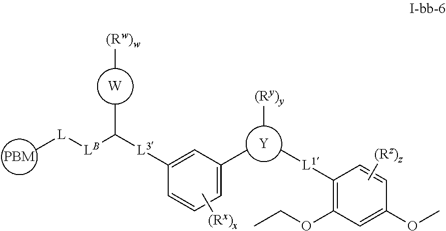

or a pharmaceutically acceptable salt thereof, wherein each of ring W, ring Y, L^{B}, L^{3′}, L^{1′}, R^{w}, R^{x}, R^{y}, R^{z}, w, x, y, z, L, and PBM is as defined above and described in embodiments herein, both singly and in combination.

In some embodiments, the present invention provides a compound of formula I-bb or I-bb′, wherein DBM is a deubiquitinase binding moiety thereby forming a compound of formula I-bb-7:

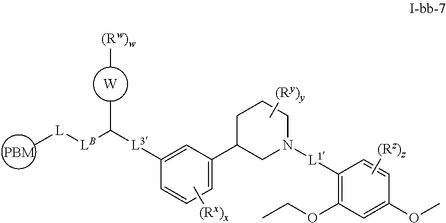

or a pharmaceutically acceptable salt thereof, wherein each of ring W, L^{B}, L^{3′}, L^{1′}, R^{w}, R^{x}, R^{y}, R^{z}, w, x, y, z, L, and PBM is as defined above and described in embodiments herein, both singly and in combination.

In some embodiments, the present invention provides a compound of formula I-bb or I-bb′, wherein DBM is a deubiquitinase binding moiety thereby forming a compound of formula I-bb-8:

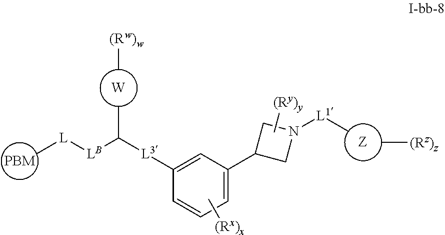

or a pharmaceutically acceptable salt thereof, wherein each of ring W, ring Z, L^{B}, L^{3′}, L^{1′}, R^{w}, R^{x}, R^{y}, R^{z}, w, x, y, z, L, and PBM is as defined above and described in embodiments herein, both singly and in combination. In some embodiments, the present invention provides a compound of formula I-bb′, wherein DBM is a deubiquitinase binding moiety thereby forming a compound of formula I-bb′-1:

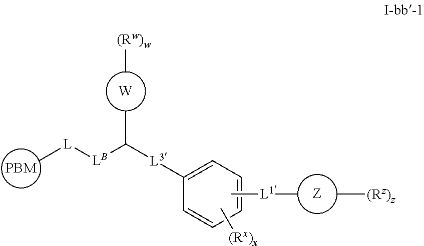

or a pharmaceutically acceptable salt thereof, wherein each of ring W, ring Z, L^{B}, L^{3′}, L^{1′}, R^{w}, R^{x}, R^{z}, w, x, z, L, and PBM is as defined above and described in embodiments herein, both singly and in combination

In some embodiments, the present invention provides a compound of formula I-bb′, wherein DBM is a deubiquitinase binding moiety thereby forming a compound of formula I-bb′-2:

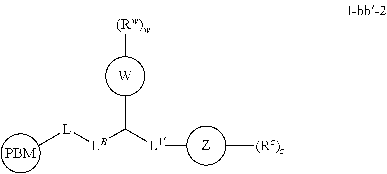

or a pharmaceutically acceptable salt thereof, wherein each of ring W, ring Z, L^{B}, L^{3′}, L^{1′}, R^{w}, R^{z}, w, z, L, and PBM is as defined above and described in embodiments herein, both singly and in combination.

In some embodiments, the present invention provides a compound of formula I-bb′, wherein DBM is a deubiquitinase binding moiety thereby forming a compound of formula I-bb′-3:

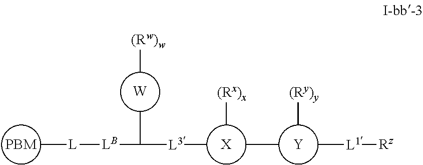

or a pharmaceutically acceptable salt thereof, wherein each of ring W, ring X, ring Y, L^{B}, L^{3′}, L^{1′}, R^{w}, R^{x}, R^{y}, R^{z}, w, x, y, L, and PBM is as defined above and described in embodiments herein, both singly and in combination.

In some embodiments, the present invention provides a compound of formula I-bb′, wherein DBM is a deubiquitinase binding moiety thereby forming a compound of formula I-bb′-4:

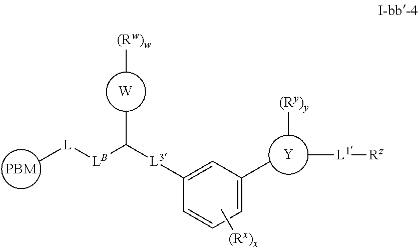

or a pharmaceutically acceptable salt thereof, wherein each of ring W, ring Y, L^{B}, L^{3′}, L^{1′}, R^{w}, R^{x}, R^{y}, R^{z}, w, x, y, L, and PBM is as defined above and described in embodiments herein, both singly and in combination.

In some embodiments, the present invention provides a compound of formula I-bb′, wherein DBM is a deubiquitinase binding moiety thereby forming a compound of formula I-bb′-5:

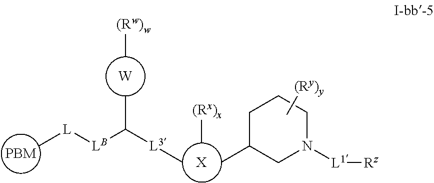

or a pharmaceutically acceptable salt thereof, wherein each of ring W, ring X, L^{B}, L^{3′}, L^{1′}, R^{w}, R^{x}, R^{y}, R^{z}, w, x, y, L, and PBM is as defined above and described in embodiments herein, both singly and in combination.

In some embodiments, the present invention provides a compound of formula I-bb′, wherein DBM is a deubiquitinase binding moiety thereby forming a compound of formula I-bb′-6:

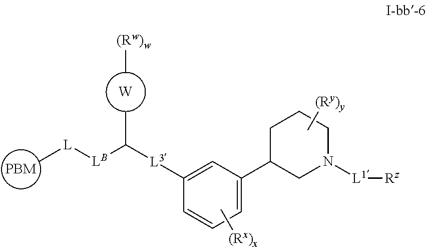

or a pharmaceutically acceptable salt thereof, wherein each of ring W, L^{B}, L^{3′}, L^{1′}, R^{w}, R^{x}, R^{y}, R^{z}, w, x, y, L, and PBM is as defined above and described in embodiments herein, both singly and in combination.

In some embodiments, the present invention provides a compound of formula I-aa, wherein DBM is a deubiquitinase binding moiety thereby forming a compound of formula I-cc:

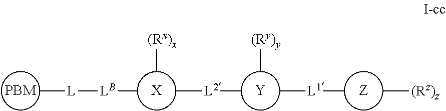

or a pharmaceutically acceptable salt thereof, wherein L and PBM are as defined above and described herein, and wherein:
Ring Z is a ring selected from phenyl, a 4-7 membered saturated or partially unsaturated carbocyclic ring, a 4-7 membered saturated or partially unsaturated heterocyclic ring with 1-3 heteroatoms independently selected from nitrogen, oxygen, and sulfur, and a 5-6 membered heteroaromatic ring with 1-4 heteroatoms independently selected from nitrogen, oxygen and sulfur;
Ring Y is a ring selected from phenylenyl, a 4-7 membered saturated or partially unsaturated carbocyclylenyl, a 4-7 membered saturated or partially unsaturated heterocyclylenyl with 1-3 heteroatoms independently selected from nitrogen, oxygen, and sulfur, and a 5-6 membered heteroarylenyl with 1-4 heteroatoms independently selected from nitrogen, oxygen and sulfur;
Ring X is a ring selected from phenylenyl, a 4-7 membered saturated or partially unsaturated carbocyclylenyl, a 4-7 membered saturated or partially unsaturated heterocyclylenyl with 1-3 heteroatoms independently selected from nitrogen, oxygen, and sulfur, and a 5-6 membered heteroarylenyl with 1-4 heteroatoms independently selected from nitrogen, oxygen and sulfur;
each of L^{B}, L^{1′}, and L^{2′} is independently a covalent bond or a bivalent, saturated or partially unsaturated, straight or branched C₁₋₅ hydrocarbon chain, wherein 0-3 methylene units of L^{B}, L^{1′}, or L^{2′} are independently replaced by —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, or —OC(O)NR—, —NRC(O)O—;
each R^{x}, R^{y}, and R^{z} is independently selected from hydrogen, R^{B}, halogen, CN, —NO₂, —OR, —SR, —NR₂, —S(O)₂R, —S(O)₂NR₂, —S(O)R, —C(O)R, —C(O)OR, —C(O)NR₂, —C(O)NROR, —OC(O)R, —OC(O)NR₂, —NRC(O)OR, —NRC(O)R, —NRC(O)N(R)₂, and —NRS(O)₂R;
each R^{B} is independently an optionally substituted group selected from C₁₋₆ aliphatic, phenyl, a 3-12 membered saturated or partially unsaturated monocyclic, bicyclic, bridged bicyclic or spirocyclic carbocyclyl or heterocyclyl with 1-3 heteroatoms independently selected from nitrogen, oxygen, and sulfur, and a 5-6 membered heteroaryl ring having 1-4 heteroatoms independently selected from nitrogen, oxygen, and sulfur;
each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same nitrogen are optionally taken together with their intervening atoms to form a 4-7 membered saturated, partially unsaturated, or heteroaryl ring having 0-3 heteroatoms, in addition to the nitrogen atom to which they are attached, independently selected from nitrogen, oxygen, and sulfur; and
x, y, and z are independently 0, 1, 2, 3, or 4.

In some embodiments, the present invention provides a compound of formula I-aa, wherein DBM is a deubiquitinase binding moiety thereby forming a compound of formula I-cc′:

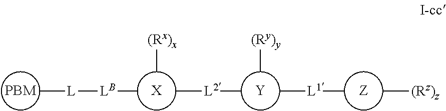

or a pharmaceutically acceptable salt thereof, wherein L and PBM are as defined above and described herein, and wherein:
Ring Z is a ring selected from phenyl, a 3-7 membered saturated or partially unsaturated monocyclic, bicyclic, bridged bicyclic or spirocyclic carbocyclic ring, a 3-7 membered saturated or partially unsaturated monocyclic, bicyclic, bridged bicyclic or spirocyclic heterocyclic ring with 1-3 heteroatoms independently selected from nitrogen, oxygen, and sulfur, and a 5-6 membered heteroaromatic ring with 1-4 heteroatoms independently selected from nitrogen, oxygen and sulfur and an 8-10 membered bicyclic heteroaromatic ring having 1-5 heteroatoms independently selected from nitrogen, oxygen, or sulfur;
Ring Y is a ring selected from phenylenyl, a 4-7 membered saturated or partially unsaturated carbocyclylenyl, a 4-8 membered saturated or partially unsaturated monocyclic, bicyclic, bridged bicyclic or spirocyclic heterocyclylenyl with 1-3 heteroatoms independently selected from nitrogen, oxygen, and sulfur, a 5-6 membered heteroarylenyl with 1-4 heteroatoms independently selected from nitrogen, oxygen and sulfur and an 8-10 membered bicyclic heterocyclylenyl having 1-5 heteroatoms independently selected from nitrogen, oxygen, or sulfur;
Ring X is a ring selected from phenylenyl, a 4-7 membered saturated or partially unsaturated carbocyclylenyl, a 4-7 membered saturated or partially unsaturated heterocyclylenyl with 1-3 heteroatoms independently selected from nitrogen, oxygen, and sulfur, and a 5-6 membered heteroarylenyl with 1-4 heteroatoms independently selected from nitrogen, oxygen and sulfur;
each of L^{B}, L^{1′}, and L^{2′} is independently a covalent bond or an optionally substituted bivalent, saturated or partially unsaturated, straight or branched C₁₋₅ hydrocarbon chain, wherein 0-3 methylene units of L^{B}, L^{1′}, or L^{2′} are independently replaced by -Cy′-, —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, or —OC(O)NR—, —NRC(O)O—;
each R^{x}, R^{y}, and R^{z} is independently selected from hydrogen, R^{B}, halogen, CN, —NO₂, —OR, —SR, —NR₂, —S(O)₂R, —S(O)₂NR₂, —S(O)R, —C(O)R, —C(O)OR, —C(O)NR₂, —C(O)NROR, —OC(O)R, —OC(O)NR₂, —NRC(O)OR, —NRC(O)R, —NRC(O)N(R)₂, and —NRS(O)₂R;
each R^{B} is independently an optionally substituted group selected from C₁₋₆ aliphatic, phenyl, a 3-12 membered saturated or partially unsaturated monocyclic, bicyclic, bridged bicyclic or spirocyclic carbocyclyl or heterocyclyl with 1-3 heteroatoms independently selected from nitrogen, oxygen, and sulfur, and a 5-6 membered heteroaryl ring having 1-4 heteroatoms independently selected from nitrogen, oxygen, and sulfur;
each -Cy′- is independently an optionally substituted bivalent ring selected from phenylenyl, an 8-10 membered bicyclic arylenyl, a 3-11 membered saturated or partially unsaturated monocyclic, bicyclic, bridged bicyclic or spirocyclic carbocyclylenyl, a 3-11 membered saturated or partially unsaturated monocyclic, bicyclic, bridged bicyclic or spirocyclic heterocyclylenyl having 1-3 heteroatoms independently selected from nitrogen, oxygen, and sulfur, a 5-6 membered heteroarylenyl having 1-4 heteroatoms independently selected from nitrogen, oxygen, and sulfur, and an 8-10 membered bicyclic heteroarylenyl having 1-5 heteroatoms independently selected from nitrogen, oxygen, and sulfur;
each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same nitrogen are optionally taken together with their intervening atoms to form a 4-7 membered saturated, partially unsaturated, or heteroaryl ring having 0-3 heteroatoms, in addition to the nitrogen atom to which they are attached, independently selected from nitrogen, oxygen, and sulfur; and
x, y, and z are independently 0, 1, 2, 3, or 4.

In some embodiments, the present invention provides a compound of formula I-cc, wherein DBM is a deubiquitinase binding moiety thereby forming a compound of formula I-cc-1:

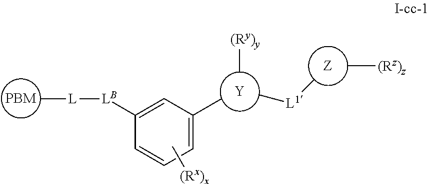

or a pharmaceutically acceptable salt thereof, wherein each of ring Y, ring Z, L^{B}, L^{1′}, R^{x}, R^{y}, R^{z}, x, y, z, L, and PBM is as defined above and described in embodiments herein, both singly and in combination.

In some embodiments, the present invention provides a compound of formula I-cc, wherein DBM is a deubiquitinase binding moiety thereby forming a compound of formula I-cc-2:

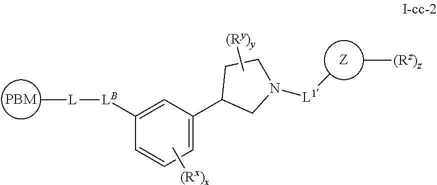

or a pharmaceutically acceptable salt thereof, wherein each of ring Z, L^{B}, L^{1′}, R^{x}, R^{y}, R^{z}, x, y, z, L, and PBM is as defined above and described in embodiments herein, both singly and in combination.

In some embodiments, the present invention provides a compound of formula I-cc, wherein DBM is a deubiquitinase binding moiety thereby forming a compound of formula I-cc-3 or I-cc-3′:

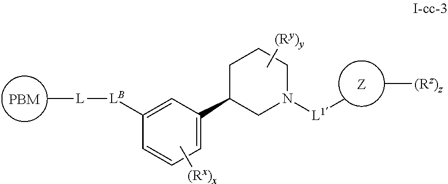

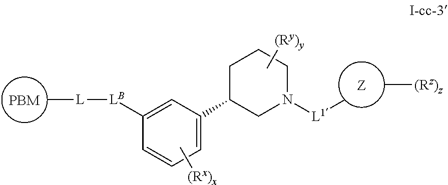

or a pharmaceutically acceptable salt thereof, wherein each of ring Z, L^{B}, L^{1′}, R^{x}, R^{y}, R^{z}, x, y, z, L, and PBM is as defined above and described in embodiments herein, both singly and in combination.

In some embodiments, the present invention provides a compound of formula I-cc, wherein DBM is a deubiquitinase binding moiety thereby forming a compound of formula I-cc-4:

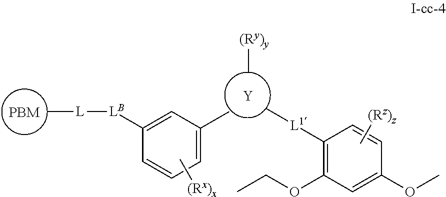

or a pharmaceutically acceptable salt thereof, wherein each of ring Y, L^{B}, L^{1′}, R^{x}, R^{y}, R^{z}, x, y, z, L, and PBM is as defined above and described in embodiments herein, both singly and in combination.

In some embodiments, the present invention provides a compound of formula I-cc, wherein DBM is a deubiquitinase binding moiety thereby forming a compound of formula I-cc-5:

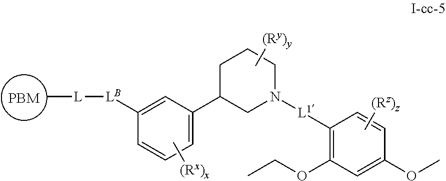

or a pharmaceutically acceptable salt thereof, wherein each of L^{B}, L^{1′}, R^{x}, R^{y}, R^{z}, x, y, z, L, and PBM is as defined above and described in embodiments herein, both singly and in combination.

In some embodiments, the present invention provides a compound of formula I-cc, wherein DBM is a deubiquitinase binding moiety thereby forming a compound of formula I-dd-1:

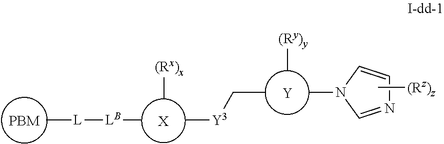

or a pharmaceutically acceptable salt thereof, wherein each of ring X, ring Y, L^{B}, R^{x}, R^{y}, R^{z}, x, y, z, L, and PBM is as defined above and described in embodiments herein, both singly and in combination, wherein Y³ is sulfur or oxygen.

In some embodiments, the present invention provides a compound of formula I-cc, wherein DBM is a deubiquitinase binding moiety thereby forming a compound of formula I-dd-2:

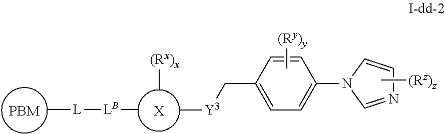

or a pharmaceutically acceptable salt thereof, wherein ring X, L^{B}, R^{x}, R^{y}, R^{z}, x, y, z, L, and PBM is as defined above and described in embodiments herein, both singly and in combination, wherein Y³ is sulfur or oxygen.

In some embodiments, the present invention provides a compound of formula I-cc, wherein DBM is a deubiquitinase binding moiety thereby forming a compound of formula I-dd-3 or I-dd-3′:

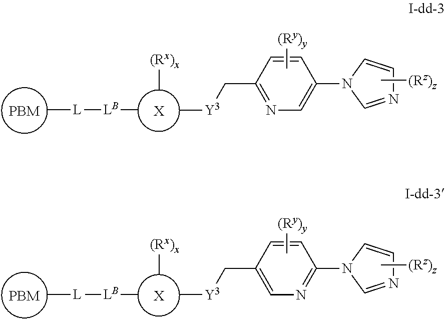

or a pharmaceutically acceptable salt thereof, wherein each of ring X, L^{B}, R^{x}, R^{y}, R^{z}, x, y, z, L, and PBM is as defined above and described in embodiments herein, both singly and in combination, wherein Y³ is sulfur or oxygen.

In some embodiments, the present invention provides a compound of formula I-cc, wherein DBM is a deubiquitinase binding moiety thereby forming a compound of formula I-dd-3:

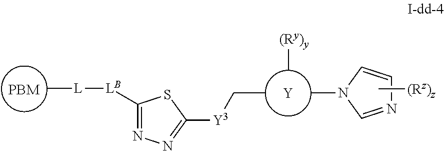

or a pharmaceutically acceptable salt thereof, wherein each of ring Y, L^{B}, R^{y}, R^{z}, y, z, L, and PBM is as defined above and described in embodiments herein, both singly and in combination, wherein Y³ is sulfur or oxygen.

In some embodiments, the present invention provides a compound of formula I-cc, wherein DBM is a deubiquitinase binding moiety thereby forming a compound of formula I-dd-5:

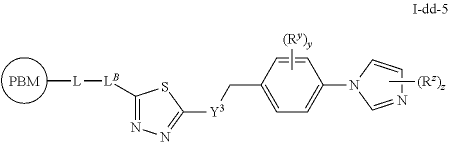

or a pharmaceutically acceptable salt thereof, wherein each of L^{B}, R^{y}, R^{z}, y, z, L, and PBM is as defined above and described in embodiments herein, both singly and in combination, wherein Y³ is sulfur or oxygen.

In some embodiments, the present invention provides a compound of formula I-cc, wherein DBM is a deubiquitinase binding moiety thereby forming a compound of formula I-ee-1:

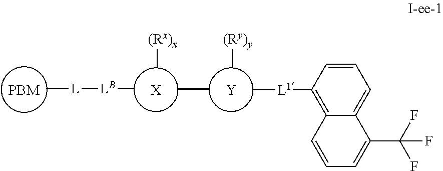

or a pharmaceutically acceptable salt thereof, wherein each of ring X, ring Y, L^{B}, L^{1′}, R^{x}, R^{y}, x, y, L, and PBM is as defined above and described in embodiments herein.

In some embodiments, the present invention provides a compound of formula I-cc, wherein DBM is a deubiquitinase binding moiety thereby forming a compound of formula I-ee-2:

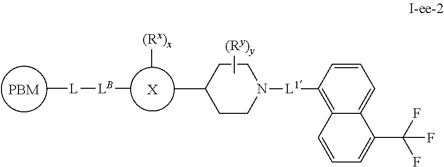

or a pharmaceutically acceptable salt thereof, wherein each of ring X, L^{B}, L^{1′}, R^{x}, R^{y}, x, y, L, and PBM is as defined above and described in embodiments herein.

In some embodiments, the present invention provides a compound of formula I-cc, wherein DBM is a deubiquitinase binding moiety thereby forming a compound of formula I-ee-3:

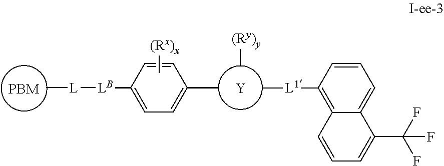

or a pharmaceutically acceptable salt thereof, wherein each of ring Y, L^{B}, L^{1′}, R^{x}, R^{y}, x, y, L, and PBM is as defined above and described in embodiments herein.

In some embodiments, the present invention provides a compound of formula I-cc′, wherein DBM is a deubiquitinase binding moiety thereby forming a compound of formula I-cc′-1:

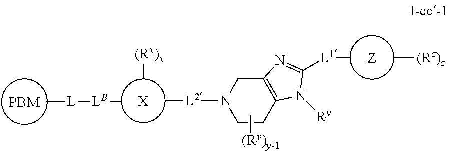

or a pharmaceutically acceptable salt thereof, wherein each of ring X, ring Z, L^{B}, L^{1′}, L², R^{x}, R^{y}, R^{z}, x, y, z, L, and PBM is as defined above and described in embodiments herein.

In some embodiments, the present invention provides a compound of formula I-cc′, wherein DBM is a deubiquitinase binding moiety thereby forming a compound of formula I-cc′-2:

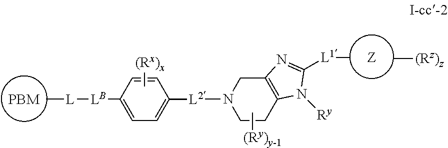

or a pharmaceutically acceptable salt thereof, wherein each of ring Z, L^{B}, L^{1′}, L^{2′}, R^{x}, R^{y}, R^{z}, x, y, z, L, and PBM is as defined above and described in embodiments herein. In some embodiments, the present invention provides a compound of formula I-aa, wherein DBM is a deubiquitinase binding moiety thereby forming a compound of formula I-ff:

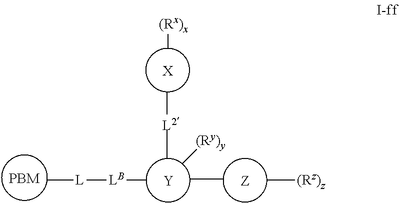

or a pharmaceutically acceptable salt thereof, wherein L and PBM are as defined above and described herein, and wherein:
Ring Z is a ring selected from phenyl, a 4-7 membered saturated or partially unsaturated carbocyclic ring, a 4-7 membered saturated or partially unsaturated heterocyclic ring with 1-3 heteroatoms independently selected from nitrogen, oxygen, and sulfur, and a 5-6 membered heteroaromatic ring with 1-4 heteroatoms independently selected from nitrogen, oxygen and sulfur;
Ring Y is a ring selected from phenylenyl, a 4-7 membered saturated or partially unsaturated carbocyclylenyl, a 4-7 membered saturated or partially unsaturated heterocyclylenyl with 1-3 heteroatoms independently selected from nitrogen, oxygen, and sulfur, and a 5-6 membered heteroarylenyl with 1-4 heteroatoms independently selected from nitrogen, oxygen and sulfur;
Ring X is a ring selected from phenylenyl, a 4-7 membered saturated or partially unsaturated carbocyclylenyl, a 4-7 membered saturated or partially unsaturated heterocyclylenyl with 1-3 heteroatoms independently selected from nitrogen, oxygen, and sulfur, and a 5-6 membered heteroarylenyl with 1-4 heteroatoms independently selected from nitrogen, oxygen and sulfur;
each of L^{B} and L^{2′} is independently a covalent bond or a covalent bond or a bivalent, saturated or partially unsaturated, straight or branched C₁₋₅ hydrocarbon chain, wherein 0-3 methylene units of L^{B} or L^{2′} are independently replaced by —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, or —OC(O)NR—, —NRC(O)O—;
each R^{x}, R^{y}, and R^{z} is independently selected from hydrogen, R^{B}, halogen, —CN, —NO₂, —OR, —SR, —NR₂, —S(O)₂R, —S(O)₂NR₂, —S(O)R, —C(O)R, —C(O)OR, —C(O)NR₂, —C(O)NROR, —OC(O)R, —OC(O)NR₂, —NRC(O)OR, —NRC(O)R, —NRC(O)N(R)₂, and —NRS(O)₂R;
each R^{B} is independently an optionally substituted group selected from C₁₋₆ aliphatic, phenyl, a 3-12 membered saturated or partially unsaturated monocyclic, bicyclic, bridged bicyclic or spirocyclic carbocyclyl or heterocyclyl with 1-3 heteroatoms independently selected from nitrogen, oxygen, and sulfur, and a 5-6 membered heteroaryl ring having 1-4 heteroatoms independently selected from nitrogen, oxygen, and sulfur;
each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same nitrogen are optionally taken together with their intervening atoms to form a 4-7 membered saturated, partially unsaturated, or heteroaryl ring having 0-3 heteroatoms, in addition to the nitrogen atom to which they are attached, independently selected from nitrogen, oxygen, and sulfur; and
x, y, and z are independently 0, 1, 2, 3, or 4.

In some embodiments, the present invention provides a compound of formula I-aa, wherein DBM is a deubiquitinase binding moiety thereby forming a compound of formula I-ff′:

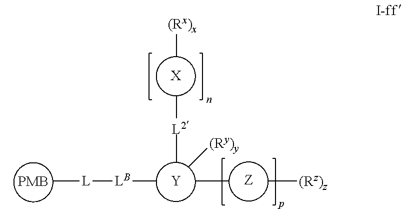

or a pharmaceutically acceptable salt thereof, wherein L and PBM are as defined above and described herein, and wherein:
Ring Z is a ring selected from phenyl, a 4-9 membered saturated or partially unsaturated monocyclic, bicyclic, bridged bicyclic or spirocyclic carbocyclic ring, a 4-9 membered saturated or partially unsaturated monocyclic, bicyclic, bridged bicyclic or spirocyclic heterocyclic ring with 1-3 heteroatoms independently selected from nitrogen, oxygen, and sulfur, a 5-6 membered heteroaromatic ring with 1-4 heteroatoms independently selected from nitrogen, oxygen and sulfur and an 8-10 membered bicyclic heteroaromatic ring having 1-5 heteroatoms independently selected from nitrogen, oxygen, or sulfur;
Ring Y is a ring selected from phenylenyl, a 4-7 membered saturated or partially unsaturated carbocyclylenyl, a 4-7 membered saturated or partially unsaturated heterocyclylenyl with 1-3 heteroatoms independently selected from nitrogen, oxygen, and sulfur, and a 5-6 membered heteroarylenyl with 1-4 heteroatoms independently selected from nitrogen, oxygen and sulfur;
Ring X is a ring selected from phenylenyl, a 3-9 membered saturated or partially unsaturated monocyclic, bicyclic, bridged bicyclic or spirocyclic carbocyclylenyl, a 3-9 membered saturated or partially unsaturated monocyclic, bicyclic, bridged bicyclic or spirocyclic heterocyclylenyl with 1-3 heteroatoms independently selected from nitrogen, oxygen, and sulfur, and a 5-6 membered heteroarylenyl with 1-4 heteroatoms independently selected from nitrogen, oxygen and sulfur;
each of L^{B} and L^{2′} is independently a covalent bond or an optionally substituted bivalent, saturated or partially unsaturated, straight or branched C₁₋₅ hydrocarbon chain, wherein 0-3 methylene units of L^{B} or L^{2′} are independently replaced by -Cy′-, —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, or —OC(O)NR—, —NRC(O)O—;
each R^{x}, R^{y}, and R^{z} is independently selected from hydrogen, R^{B}, halogen, —CN, —NO₂, —OR, —SR, —NR₂, —S(O)₂R, —S(O)₂NR₂, —S(O)R, —C(O)R, —C(O)OR, —C(O)NR₂, —C(O)NROR, —OC(O)R, —OC(O)NR₂, —NRC(O)OR, —NRC(O)R, —NRC(O)N(R)₂, and —NRS(O)₂R;
each R^{B} is independently an optionally substituted group selected from C₁₋₆ aliphatic, phenyl, a 3-12 membered saturated or partially unsaturated monocyclic, bicyclic, bridged bicyclic or spirocyclic carbocyclyl or heterocyclyl with 1-3 heteroatoms independently selected from nitrogen, oxygen, and sulfur, and a 5-6 membered heteroaryl ring having 1-4 heteroatoms independently selected from nitrogen, oxygen, and sulfur;
each -Cy′- is independently an optionally substituted bivalent ring selected from phenylenyl, an 8-10 membered bicyclic arylenyl, a 3-11 membered saturated or partially unsaturated monocyclic, bicyclic, bridged bicyclic or spirocyclic carbocyclylenyl, a 3-11 membered saturated or partially unsaturated monocyclic, bicyclic, bridged bicyclic or spirocyclic heterocyclylenyl having 1-3 heteroatoms independently selected from nitrogen, oxygen, and sulfur, a 5-6 membered heteroarylenyl having 1-4 heteroatoms independently selected from nitrogen, oxygen, and sulfur, and an 8-10 membered bicyclic heteroarylenyl having 1-5 heteroatoms independently selected from nitrogen, oxygen, and sulfur;
each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same nitrogen are optionally taken together with their intervening atoms to form a 4-7 membered saturated, partially unsaturated, or heteroaryl ring having 0-3 heteroatoms, in addition to the nitrogen atom to which they are attached, independently selected from nitrogen, oxygen, and sulfur; n and p are independently 0 or 1; and
x, y, and z are independently 0, 1, 2, 3, or 4.

In some embodiments, the present invention provides a compound of formula I-ff or I-ff′, wherein DBM is a deubiquitinase binding moiety thereby forming a compound of formula I-ff-2:

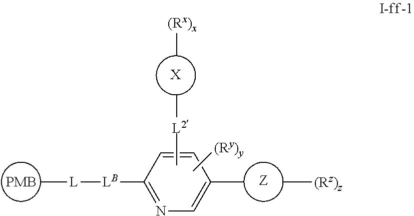

or a pharmaceutically acceptable salt thereof, wherein each of ring X, ring Z, L^{B}, L^{2′}, R^{x}, R^{y}, R^{z}, x, y, z, L, and PBM is as defined above and described in embodiments herein, both singly and in combination.

In some embodiments, the present invention provides a compound of formula I-ff or I-ff′, wherein DBM is a deubiquitinase binding moiety thereby forming a compound of formula I-ff-2′:

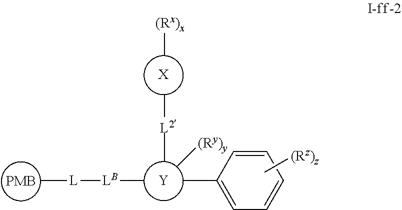

or a pharmaceutically acceptable salt thereof, wherein each of ring X, ring Y, L^{B}, L^{2′}, R^{x}, R^{y}, R^{z}, x, y, z, L, and PBM is as defined above and described in embodiments herein, both singly and in combination.

In some embodiments, the present invention provides a compound of formula I-ff or I-ff′, wherein DBM is a deubiquitinase binding moiety thereby forming a compound of formula I-ff-3:

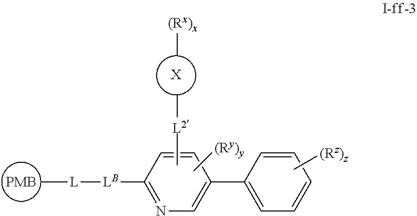

or a pharmaceutically acceptable salt thereof, wherein each of ring X, L^{B}, L^{2′}, R^{x}, R^{y}, R^{z}, x, y, z, L, and PBM is as defined above and described in embodiments herein, both singly and in combination.

In some embodiments, the present invention provides a compound of formula I-ff or I-ff′, wherein DBM is a deubiquitinase binding moiety thereby forming a compound of formula I-ff-4:

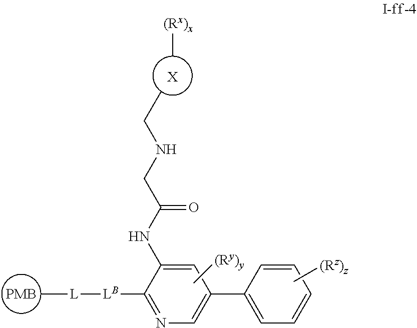

or a pharmaceutically acceptable salt thereof, wherein each of ring X, L^{B}, R^{x}, R^{y}, R^{z}, x, y, z, L, and PBM is as defined above and described in embodiments herein, both singly and in combination.

In some embodiments, the present invention provides a compound of formula I-ff′, wherein DBM is a deubiquitinase binding moiety thereby forming a compound of formula I-ff′-1:

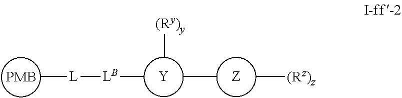

or a pharmaceutically acceptable salt thereof, wherein each of ring Y, ring Z, L^{B}, R^{y}, R^{z}, y, z, L, and PBM is as defined above and described in embodiments herein, both singly and in combination.

In some embodiments, the present invention provides a compound of formula I-ff′, wherein DBM is a deubiquitinase binding moiety thereby forming a compound of formula I-ff′-2:

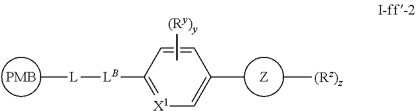

or a pharmaceutically acceptable salt thereof, wherein each of ring Y, ring Z, L^{B}, R^{y}, R^{z}, y, z, L, and PBM is as defined above and described in embodiments herein, both singly and in combination; and wherein X¹ is carbon or nitrogen.

In some embodiments, the present invention provides a compound of formula I-ff′, wherein DBM is a deubiquitinase binding moiety thereby forming a compound of formula I-ff′-3:

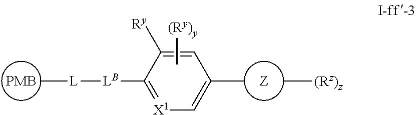

or a pharmaceutically acceptable salt thereof, wherein each of ring Z, L^{B}, R^{y}, R^{z}, y, z, L, and PBM is as defined above and described in embodiments herein, both singly and in combination.

In some embodiments, the present invention provides a compound of formula I-ff′, wherein DBM is a deubiquitinase binding moiety thereby forming a compound of formula I-ff′-4:

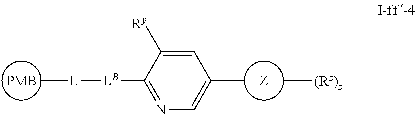

or a pharmaceutically acceptable salt thereof, wherein each of ring Z, L^{B}, R^{x}, R^{z}, y, z, L, and PBM is as defined above and described in embodiments herein, both singly and in combination.

In some embodiments, the present invention provides a compound of formula I-ff′, wherein DBM is a deubiquitinase binding moiety thereby forming a compound of formula I-ff′-5:

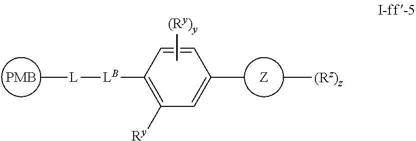

or a pharmaceutically acceptable salt thereof, wherein each of ring Z, L^{B}, R^{y}, R^{z}, y, z, L, and PBM is as defined above and described in embodiments herein, both singly and in combination. In some embodiments, the present invention provides a compound of formula I-ff′, wherein DBM is a deubiquitinase binding moiety thereby forming a compound of formula I-ff′-6:

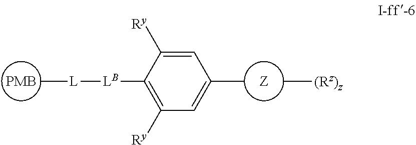

or a pharmaceutically acceptable salt thereof, wherein each of ring Z, L^{B}, R^{y}, R^{z}, y, z, L, and PBM is as defined above and described in embodiments herein, both singly and in combination.

In some embodiments, the present invention provides a compound of formula I-aa, wherein DBM is a deubiquitinase binding moiety thereby forming a compound of formula I-gg:

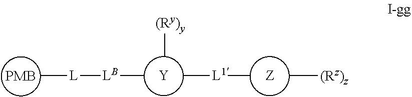

or a pharmaceutically acceptable salt thereof, wherein L and PBM are as defined above and described herein, and wherein:
Ring Z is a ring selected from phenyl, a 4-7 membered saturated or partially unsaturated carbocyclic ring, a 4-7 membered saturated or partially unsaturated heterocyclic ring with 1-3 heteroatoms independently selected from nitrogen, oxygen, and sulfur, and a 5-6 membered heteroaromatic ring with 1-4 heteroatoms independently selected from nitrogen, oxygen and sulfur;
Ring Y is a ring selected from phenylenyl, a 4-7 membered saturated or partially unsaturated carbocyclylenyl, a 4-7 membered saturated or partially unsaturated heterocyclylenyl with 1-3 heteroatoms independently selected from nitrogen, oxygen, and sulfur, and a 5-6 membered heteroarylenyl with 1-4 heteroatoms independently selected from nitrogen, oxygen and sulfur;
each of L^{B} and L^{1′} is independently a covalent bond or a covalent bond or a bivalent, saturated or partially unsaturated, straight or branched C₁₋₅ hydrocarbon chain, wherein 0-3 methylene units of L^{B} or L^{1′} are independently replaced by —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, or —OC(O)NR—, —NRC(O)O—;
each R^{y}, and R^{z} is independently selected from hydrogen, R^{B}, halogen, CN, —NO₂, —OR, —SR, —NR₂, —S(O)₂R, —S(O)₂NR₂, —S(O)R, —C(O)R, —C(O)OR, —C(O)NR₂, —C(O)NROR, —OC(O)R, —OC(O)NR₂, —NRC(O)OR, —NRC(O)R, —NRC(O)N(R)₂, and —NRS(O)₂R;
each R^{B} is independently an optionally substituted group selected from C₁₋₆ aliphatic, phenyl, a 3-12 membered saturated or partially unsaturated monocyclic, bicyclic, bridged bicyclic or spirocyclic carbocyclyl or heterocyclyl with 1-3 heteroatoms independently selected from nitrogen, oxygen, and sulfur, and a 5-6 membered heteroaryl ring having 1-4 heteroatoms independently selected from nitrogen, oxygen, and sulfur;
each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same nitrogen are optionally taken together with their intervening atoms to form a 4-7 membered saturated, partially unsaturated, or heteroaryl ring having 0-3 heteroatoms, in addition to the nitrogen atom to which they are attached, independently selected from nitrogen, oxygen, and sulfur; and
y and z are independently 0, 1, 2, 3, or 4.

In some embodiments, the present invention provides a compound of formula I-gg, wherein DBM is a deubiquitinase binding moiety thereby forming a compound of formula I-gg-1:

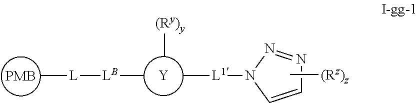

or a pharmaceutically acceptable salt thereof, wherein each of ring Y, L^{B}, L^{1′}, R^{y}, R^{z}, y, z, L, and PBM is as defined above and described in embodiments herein, both singly and in combination.

In some embodiments, the present invention provides a compound of formula I-gg wherein DBM is a deubiquitinase binding moiety thereby forming a compound of formula I-gg-2:

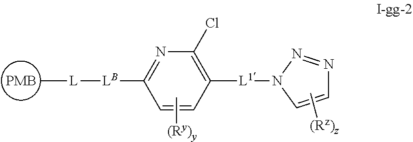

or a pharmaceutically acceptable salt thereof, wherein each of L^{B}, L^{1′}, R^{y}, R^{z}, y, z, L, and PBM is as defined above and described in embodiments herein, both singly and in combination.

In some embodiments, the present invention provides a compound of formula I-gg, wherein DBM is a deubiquitinase binding moiety thereby forming a compound of formula I-gg-3:

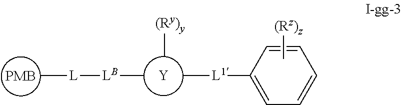

or a pharmaceutically acceptable salt thereof, wherein each of ring Y, L^{B}, L^{1′}, R^{y}, R^{z}, y, z, L, and PBM is as defined above and described in embodiments herein, both singly and in combination.

In some embodiments, the present invention provides a compound of formula I-gg, wherein DBM is a deubiquitinase binding moiety thereby forming a compound of formula I-gg-4:

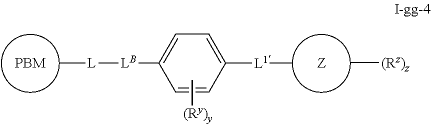

or a pharmaceutically acceptable salt thereof, wherein each of ring Z, L^{B}, L^{1′}, R^{x}, R^{z}, y, z, L, and PBM is as defined above and described in embodiments herein, both singly and in combination.

In some embodiments, the present invention provides a compound of formula I-gg, wherein DBM is a deubiquitinase binding moiety thereby forming a compound of formula I-gg-5:

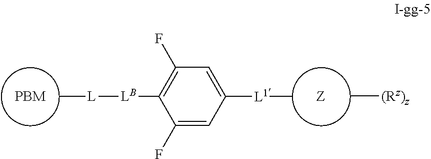

or a pharmaceutically acceptable salt thereof, wherein each of ring Z, L^{B}, L^{1′}, R^{z}, z, L, and PBM is as defined above and described in embodiments herein, both singly and in combination.

In some embodiments, the present invention provides a compound of formula I-aa, wherein DBM is a deubiquitinase binding moiety thereby forming a compound of formula I-hh:

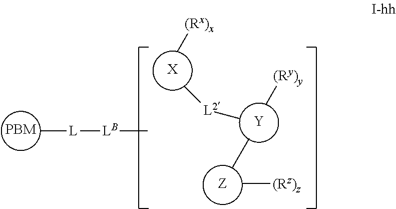

or a pharmaceutically acceptable salt thereof, wherein L and PBM are as defined above and described herein, and wherein:
Ring Z is a ring selected from phenyl, a 4-7 membered saturated or partially unsaturated carbocyclic ring, a 4-7 membered saturated or partially unsaturated heterocyclic ring with 1-3 heteroatoms independently selected from nitrogen, oxygen, and sulfur, a 5-6 membered heteroaromatic ring with 1-4 heteroatoms independently selected from nitrogen, oxygen and sulfur, and an 8-10 membered bicyclic heteroaromatic ring having 1-5 heteroatoms independently selected from nitrogen, oxygen, or sulfur;
Ring Y is a ring selected from phenylenyl, a 4-7 membered saturated or partially unsaturated carbocyclylenyl, a 4-7 membered saturated or partially unsaturated heterocyclylenyl with 1-3 heteroatoms independently selected from nitrogen, oxygen, and sulfur, and a 5-6 membered heteroarylenyl with 1-4 heteroatoms independently selected from nitrogen, oxygen and sulfur;
Ring X is a ring selected from phenylenyl, a 4-7 membered saturated or partially unsaturated carbocyclylenyl, a 4-7 membered saturated or partially unsaturated heterocyclylenyl with 1-3 heteroatoms independently selected from nitrogen, oxygen, and sulfur, and a 5-6 membered heteroarylenyl with 1-4 heteroatoms independently selected from nitrogen, oxygen and sulfur;
each L^{B} and L^{2′} is independently a covalent bond or a bivalent, saturated or partially unsaturated, straight or branched C₁₋₅ hydrocarbon chain, wherein 0-3 methylene units of L^{B} or L^{2′} are independently replaced by —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, or —OC(O)NR—, —NRC(O)O—;
each R^{x}, R^{y}, and R^{z} is independently selected from hydrogen, R^{B}, halogen, CN, —NO₂, —OR, —SR, —NR₂, —S(O)₂R, —S(O)₂NR₂, —S(O)R, —C(O)R, —C(O)OR, —C(O)NR₂, —C(O)NROR, —OC(O)R, —OC(O)NR₂, —NRC(O)OR, —NRC(O)R, —NRC(O)N(R)₂, and —NRS(O)₂R;
each R^{B} is independently an optionally substituted group selected from C₁₋₆ aliphatic, phenyl, a 3-12 membered saturated or partially unsaturated monocyclic, bicyclic, bridged bicyclic or spirocyclic carbocyclyl or heterocyclyl with 1-3 heteroatoms independently selected from nitrogen, oxygen, and sulfur, and a 5-6 membered heteroaryl ring having 1-4 heteroatoms independently selected from nitrogen, oxygen, and sulfur;
each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same nitrogen are optionally taken together with their intervening atoms to form a 4-7 membered saturated, partially unsaturated, or heteroaryl ring having 0-3 heteroatoms, in addition to the nitrogen atom to which they are attached, independently selected from nitrogen, oxygen, and sulfur; and
x, y, and z are independently 0, 1, 2, 3, or 4.

In some embodiments, the present invention provides a compound of formula I-aa, wherein DBM is a deubiquitinase binding moiety thereby forming a compound of formula I-hh′:

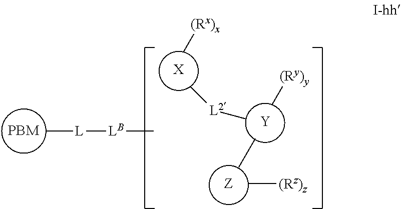

or a pharmaceutically acceptable salt thereof, wherein L and PBM are as defined above and described herein, and wherein:
Ring Z is a ring selected from phenyl, a 4-7 membered saturated or partially unsaturated carbocyclic ring, a 4-7 membered saturated or partially unsaturated heterocyclic ring with 1-3 heteroatoms independently selected from nitrogen, oxygen, and sulfur, a 5-6 membered heteroaromatic ring with 1-4 heteroatoms independently selected from nitrogen, oxygen and sulfur, and an 8-10 membered bicyclic heteroaromatic ring having 1-5 heteroatoms independently selected from nitrogen, oxygen, or sulfur;
Ring Y is a ring selected from phenylenyl, a 4-7 membered saturated or partially unsaturated carbocyclylenyl, a 4-7 membered saturated or partially unsaturated heterocyclylenyl with 1-3 heteroatoms independently selected from nitrogen, oxygen, and sulfur, and a 5-6 membered heteroarylenyl with 1-4 heteroatoms independently selected from nitrogen, oxygen and sulfur;
Ring X is a ring selected from phenylenyl, a 4-7 membered saturated or partially unsaturated carbocyclylenyl, a 4-7 membered saturated or partially unsaturated heterocyclylenyl with 1-3 heteroatoms independently selected from nitrogen, oxygen, and sulfur, and a 5-6 membered heteroarylenyl with 1-4 heteroatoms independently selected from nitrogen, oxygen and sulfur;
each L^{B} and L^{2′} is independently a covalent bond or a bivalent, saturated or partially unsaturated, straight or branched C₁₋₅ hydrocarbon chain, wherein 0-3 methylene units of L^{B} or L^{2′} are independently replaced by -Cy, —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, or —OC(O)NR—, —NRC(O)O—;
each R^{x}, R^{y}, and R^{z} is independently selected from hydrogen, R^{B}, halogen, CN, —NO₂, —OR, —SR, —NR₂, —S(O)₂R, —S(O)₂NR₂, —S(O)R, —C(O)R, —C(O)OR, —C(O)NR₂, —C(O)NROR, —OC(O)R, —OC(O)NR₂, —NRC(O)OR, —NRC(O)R, —NRC(O)N(R)₂, and —NRS(O)₂R;
each R^{B} is independently an optionally substituted group selected from C₁₋₆ aliphatic, phenyl, a 3-12 membered saturated or partially unsaturated monocyclic, bicyclic, bridged bicyclic or spirocyclic carbocyclyl or heterocyclyl with 1-3 heteroatoms independently selected from nitrogen, oxygen, and sulfur, and a 5-6 membered heteroaryl ring having 1-4 heteroatoms independently selected from nitrogen, oxygen, and sulfur;
each -Cy- is independently an optionally substituted bivalent ring selected from phenylenyl, an 8-10 membered bicyclic arylenyl, a 3-11 membered saturated or partially unsaturated monocyclic, bicyclic, bridged bicyclic or spirocyclic carbocyclylenyl, a 3-11 membered saturated or partially unsaturated monocyclic, bicyclic, bridged bicyclic or spirocyclic heterocyclylenyl having 1-3 heteroatoms independently selected from nitrogen, oxygen, and sulfur, a 5-6 membered heteroarylenyl having 1-4 heteroatoms independently selected from nitrogen, oxygen, and sulfur, and an 8-10 membered bicyclic heteroarylenyl having 1-5 heteroatoms independently selected from nitrogen, oxygen, and sulfur;
each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same nitrogen are optionally taken together with their intervening atoms to form a 4-7 membered saturated, partially unsaturated, or heteroaryl ring having 0-3 heteroatoms, in addition to the nitrogen atom to which they are attached, independently selected from nitrogen, oxygen, and sulfur; and
x, y, and z are independently 0, 1, 2, 3, or 4.

In some embodiments, the present invention provides a compound of formula I-hh or I-hh′, wherein DBM is a deubiquitinase binding moiety thereby forming a compound of formula I-hh-1:

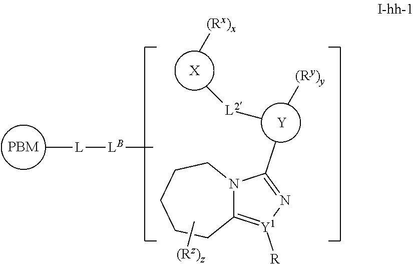

or a pharmaceutically acceptable salt thereof, wherein each of ring X, ring Y, L^{B}, L², R^{x}, R^{y}, R^{z}, R, x, y, z, L, and PBM is as defined above and described in embodiments herein, both singly and in combination; wherein Y^{1′} is nitrogen or carbon.

In some embodiments, the present invention provides a compound of formula I-hh or I-hh′, wherein DBM is a deubiquitinase binding moiety thereby forming a compound of formula I-hh-2:

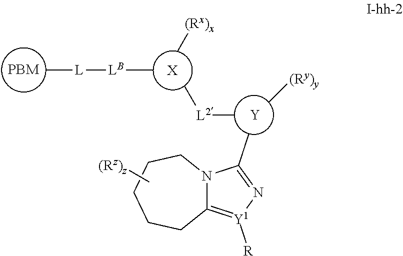

or a pharmaceutically acceptable salt thereof, wherein each of ring X, ring Y, L^{B}, L^{2′}, R^{x}, R^{y}, R^{z}, R, x, y, z, L, and PBM is as defined above and described in embodiments herein, both singly and in combination; wherein Y^{1′} is nitrogen or carbon.

In some embodiments, the present invention provides a compound of formula I-hh or I-hh′, wherein DBM is a deubiquitinase binding moiety thereby forming a compound of formula I-hh-3:

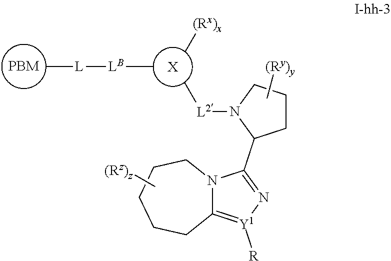

or a pharmaceutically acceptable salt thereof, wherein each of ring X, L^{B}, L^{2′}, R^{x}, R^{y}, R^{z}, R, x, y, z, L, and PBM is as defined above and described in embodiments herein, both singly and in combination; wherein Y¹ is nitrogen or carbon.

In some embodiments, the present invention provides a compound of formula I-hh or I-hh′, wherein DBM is a deubiquitinase binding moiety thereby forming a compound of formula I-hh-4 or I-hh′-4:

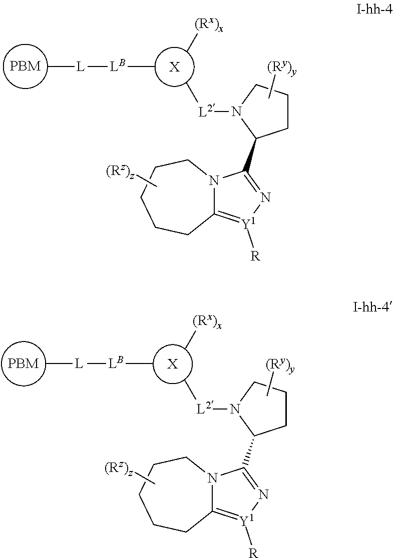

or a pharmaceutically acceptable salt thereof, wherein each of ring X, L^{B}, L^{2′}, R^{x}, R^{y}, R^{z}, R, x, y, z, L, and PBM is as defined above and described in embodiments herein, both singly and in combination; wherein Y¹ is nitrogen or carbon.

In some embodiments, the present invention provides a compound of formula I-aa, wherein DBM is a deubiquitinase binding moiety thereby forming a compound of formula I-ii:

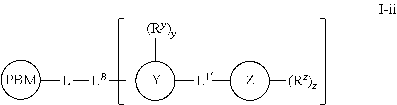

or a pharmaceutically acceptable salt thereof, wherein L and PBM are as defined above and described herein, and wherein:
Ring Z is a ring selected from phenyl, a 4-7 membered saturated or partially unsaturated carbocyclic ring, a 4-7 membered saturated or partially unsaturated heterocyclic ring with 1-3 heteroatoms independently selected from nitrogen, oxygen, and sulfur, and a 5-6 membered heteroaromatic ring with 1-4 heteroatoms independently selected from nitrogen, oxygen and sulfur;
Ring Y is a ring selected from phenylenyl, a 4-7 membered saturated or partially unsaturated carbocyclylenyl, a 4-7 membered saturated or partially unsaturated heterocyclylenyl with 1-3 heteroatoms independently selected from nitrogen, oxygen, and sulfur, a 5-6 membered heteroarylenyl with 1-4 heteroatoms independently selected from nitrogen, oxygen and sulfur and an 8-10 membered bicyclic heterocyclylenyl having 1-5 heteroatoms independently selected from nitrogen, oxygen, or sulfur;
each L^{B} and L^{1′} is a covalent bond or a bivalent, saturated or partially unsaturated, straight or branched C₁₋₅ hydrocarbon chain, wherein 0-3 methylene units of L^{B} or L^{1′} are independently replaced by —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, or —OC(O)NR—, —NRC(O)O—;
each R^{y}, and R^{z} is independently selected from hydrogen, R^{B}, halogen, CN, —NO₂, —OR, —SR, —NR₂, —S(O)₂R, —S(O)₂NR₂, —S(O)R, —C(O)R, —C(O)OR, —C(O)NR₂, —C(O)NROR, —OC(O)R, —OC(O)NR₂, —NRC(O)OR, —NRC(O)R, —NRC(O)N(R)₂, and —NRS(O)₂R;
each R^{B} is independently an optionally substituted group selected from C₁₋₆ aliphatic, phenyl, a 3-12 membered saturated or partially unsaturated monocyclic, bicyclic, bridged bicyclic or spirocyclic carbocyclyl or heterocyclyl with 1-3 heteroatoms independently selected from nitrogen, oxygen, and sulfur, and a 5-6 membered heteroaryl ring having 1-4 heteroatoms independently selected from nitrogen, oxygen, and sulfur;
each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same nitrogen are optionally taken together with their intervening atoms to form a 4-7 membered saturated, partially unsaturated, or heteroaryl ring having 0-3 heteroatoms, in addition to the nitrogen atom to which they are attached, independently selected from nitrogen, oxygen, and sulfur; and y, and z are independently 0, 1, 2, 3, or 4.

In some embodiments, the present invention provides a compound of formula I-ii, wherein DBM is a deubiquitinase binding moiety thereby forming a compound of formula I-ii-1:

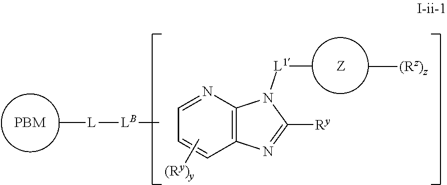

or a pharmaceutically acceptable salt thereof, wherein each of ring Z, L^{1′}, L^{B}, R^{y}, R^{z}, y, z, L, and PBM is as defined above and described in embodiments herein, both singly and in combination.

In some embodiments, the present invention provides a compound of formula I-ii, wherein DBM is a deubiquitinase binding moiety thereby forming a compound of formula I-ii-2:

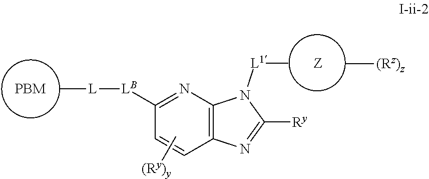

or a pharmaceutically acceptable salt thereof, wherein each of ring Z, L^{1′}, L^{B}, R^{y}, R^{z}, y, z, L, and PBM is as defined above and described in embodiments herein, both singly and in combination.

In some embodiments, the present invention provides a compound of formula I-ii, wherein DBM is a deubiquitinase binding moiety thereby forming a compound of formula I-jj-1:

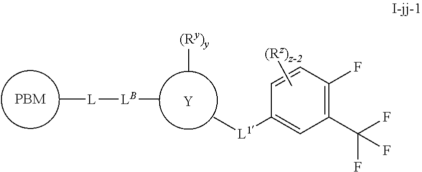

or a pharmaceutically acceptable salt thereof, wherein each of ring Y, L^{1′}, L^{B}, R^{x}, R^{z}, y, z, L, and PBM is as defined above and described in embodiments herein, both singly and in combination.

In some embodiments, the present invention provides a compound of formula I-aa, wherein DBM is a deubiquitinase binding moiety thereby forming a compound of formula I-kk:

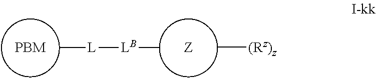

or a pharmaceutically acceptable salt thereof, wherein L and PBM are as defined above and described herein, and wherein:
Ring Z is a ring selected from phenyl, a 4-7 membered saturated or partially unsaturated carbocyclic ring, an 8-10 membered bicyclic aromatic carbocyclic ring, a 4-8 membered saturated or partially unsaturated monocyclic heterocyclic ring having 1-2 heteroatoms independently selected from nitrogen, oxygen, or sulfur, a 5-6 membered monocyclic heteroaromatic ring having 1-4 heteroatoms independently selected from nitrogen, oxygen, or sulfur, and an 8-10 membered bicyclic heteroaromatic ring having 1-5 heteroatoms independently selected from nitrogen, oxygen, or sulfur;
each R^{z} is independently selected from hydrogen, R^{B}, halogen, CN, —NO₂, —OR, —SR, —NR₂, —S(O)₂R, —S(O)₂NR₂, —S(O)R, —C(O)R, —C(O)OR, —C(O)NR₂, —C(O)NROR, —OC(O)R, —OC(O)NR₂, —NRC(O)OR, —NRC(O)R, —NRC(O)N(R)₂, and —NRS(O)₂R;
L^{B} is a covalent bond or a bivalent, saturated or partially unsaturated, straight or branched C₁₋₅ hydrocarbon chain, wherein 0-3 methylene units of L^{B} are independently replaced by —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, or —OC(O)NR—, —NRC(O)O—;
each R^{B} is independently an optionally substituted group selected from C₁₋₆ aliphatic, phenyl, a 3-12 membered saturated or partially unsaturated monocyclic, bicyclic, bridged bicyclic or spirocyclic carbocyclyl or heterocyclyl with 1-3 heteroatoms independently selected from nitrogen, oxygen, and sulfur, and a 5-6 membered heteroaryl ring having 1-4 heteroatoms independently selected from nitrogen, oxygen, and sulfur;
each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same nitrogen are optionally taken together with their intervening atoms to form a 4-7 membered saturated, partially unsaturated, or heteroaryl ring having 0-3 heteroatoms, in addition to the nitrogen atom to which they are attached, independently selected from nitrogen, oxygen, and sulfur; and
z is 0, 1, 2, 3, or 4.

In some embodiments, the present invention provides a compound of formula I-aa, wherein DBM is a deubiquitinase binding moiety thereby forming a compound of formula I-kk-1:

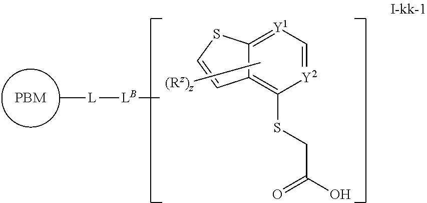

or a pharmaceutically acceptable salt thereof, wherein each of R^{z}, z, L^{B}, L, and PBM is as defined above and described in embodiments herein, both singly and in combination, wherein Y¹ and Y² are independently carbon or nitrogen.

In some embodiments, the present invention provides a compound of formula I-kk, wherein DBM is a deubiquitinase binding moiety thereby forming a compound of formula I-kk-2:

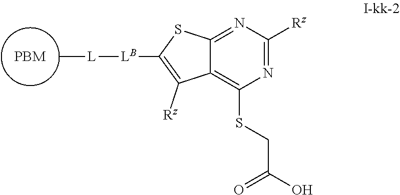

or a pharmaceutically acceptable salt thereof, wherein each of R^{z}, L^{B}, L, and PBM is as defined above and described in embodiments herein, both singly and in combination.

In some embodiments, the present invention provides a compound of formula I-aa, wherein DBM is a deubiquitinase binding moiety thereby forming a compound of formula I-11:

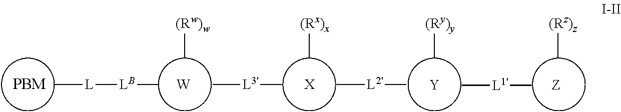

or a pharmaceutically acceptable salt thereof, wherein L and PBM are as defined above and described herein, and wherein:
Ring Z is a ring selected from phenyl, a 4-7 membered saturated or partially unsaturated carbocyclic ring, a 4-7 membered saturated or partially unsaturated heterocyclic ring with 1-3 heteroatoms independently selected from nitrogen, oxygen, and sulfur, and a 5-6 membered heteroaromatic ring with 1-4 heteroatoms independently selected from nitrogen, oxygen and sulfur;
Ring Y is a ring selected from phenylenyl, a 4-7 membered saturated or partially unsaturated carbocyclylenyl, a 4-7 membered saturated or partially unsaturated heterocyclylenyl with 1-3 heteroatoms independently selected from nitrogen, oxygen, and sulfur, and a 5-6 membered heteroarylenyl with 1-4 heteroatoms independently selected from nitrogen, oxygen and sulfur;
Ring X is a ring selected from phenylenyl, a 4-7 membered saturated or partially unsaturated carbocyclylenyl, a 4-7 membered saturated or partially unsaturated heterocyclylenyl with 1-3 heteroatoms independently selected from nitrogen, oxygen, and sulfur, and a 5-6 membered heteroarylenyl with 1-4 heteroatoms independently selected from nitrogen, oxygen and sulfur;
Ring W is a ring selected from phenylenyl, a 4-7 membered saturated or partially unsaturated carbocyclylenyl, a 4-7 membered saturated or partially unsaturated heterocyclylenyl with 1-3 heteroatoms independently selected from nitrogen, oxygen, and sulfur, and a 5-6 membered heteroarylenyl with 1-4 heteroatoms independently selected from nitrogen, oxygen and sulfur;
each L^{B}, L^{1′}, and L^{3′} is independently a covalent bond or a bivalent, saturated or partially unsaturated, straight or branched C₁₋₅ hydrocarbon chain, wherein 0-3 methylene units of L^{B}, L^{1′}, or L^{3′} are independently replaced by —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, or —OC(O)NR—, —NRC(O)O—;
each R^{w}, R^{x}, R^{y}, and R^{z} is independently selected from hydrogen, R^{B}, halogen, —CN, —NO₂, —OR, —SR, —NR₂, —S(O)₂R, —S(O)₂NR₂, —S(O)R, —C(O)R, —C(O)OR, —C(O)NR₂, —C(O)NROR, —OC(O)R, —OC(O)NR₂, —NRC(O)OR, —NRC(O)R, —NRC(O)N(R)₂, and —NRS(O)₂R;
each R^{B} is independently an optionally substituted group selected from C₁₋₆ aliphatic, phenyl, a 3-12 membered saturated or partially unsaturated monocyclic, bicyclic, bridged bicyclic or spirocyclic carbocyclyl or heterocyclyl with 1-3 heteroatoms independently selected from nitrogen, oxygen, and sulfur, and a 5-6 membered heteroaryl ring having 1-4 heteroatoms independently selected from nitrogen, oxygen, and sulfur;
each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same nitrogen are optionally taken together with their intervening atoms to form a 4-7 membered saturated, partially unsaturated, or heteroaryl ring having 0-3 heteroatoms, in addition to the nitrogen atom to which they are attached, independently selected from nitrogen, oxygen, and sulfur; and
w, x, y, and z are independently 0, 1, 2, 3, or 4.

In some embodiments, the present invention provides a compound of formula I, wherein DBM is a deubiquitinase binding moiety thereby forming a compound of formula I-11′:

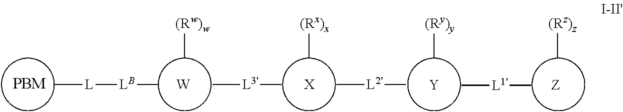

or a pharmaceutically acceptable salt thereof, wherein L and PBM are as defined above and described herein, and wherein:
Ring Z is a ring selected from phenylenyl, a 3-7 membered saturated or partially unsaturated monocyclic, bicyclic, bridged bicyclic or spirocyclic carbocyclylenyl, a 3-7 membered saturated or partially unsaturated monocyclic, bicyclic, bridged bicyclic or spirocyclic heterocyclylenyl with 1-3 heteroatoms independently selected from nitrogen, oxygen, and sulfur, and a 5-6 membered heteroarylenyl with 1-4 heteroatoms independently selected from nitrogen, oxygen and sulfur;
Ring Y is a ring selected from phenylenyl, a 4-7 membered saturated or partially unsaturated carbocyclylenyl, a 4-7 membered saturated or partially unsaturated heterocyclylenyl with 1-3 heteroatoms independently selected from nitrogen, oxygen, and sulfur, and a 5-6 membered heteroarylenyl with 1-4 heteroatoms independently selected from nitrogen, oxygen and sulfur;
Ring X is a ring selected from phenylenyl, a 4-7 membered saturated or partially unsaturated carbocyclylenyl, a 4-7 membered saturated or partially unsaturated heterocyclylenyl with 1-3 heteroatoms independently selected from nitrogen, oxygen, and sulfur, and a 5-6 membered heteroarylenyl with 1-4 heteroatoms independently selected from nitrogen, oxygen and sulfur;
Ring W is a ring selected from phenylenyl, a 4-7 membered saturated or partially unsaturated carbocyclylenyl, a 4-7 membered saturated or partially unsaturated heterocyclylenyl with 1-3 heteroatoms independently selected from nitrogen, oxygen, and sulfur, and a 5-6 membered heteroarylenyl with 1-4 heteroatoms independently selected from nitrogen, oxygen and sulfur;
each L^{B}, L^{1′}, and L^{3′} is independently a covalent bond or an optionally substituted bivalent, saturated or partially unsaturated, straight or branched C₁₋₅ hydrocarbon chain, wherein 0-3 methylene units of L^{B}, L^{1′}, or L^{3′} are independently replaced by —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, or —OC(O)NR—, —NRC(O)O—;
each R^{w}, R^{x}, R^{y}, and R^{z} is independently selected from hydrogen, R^{B}, halogen, —CN, —NO₂, —OR, —SR, —NR₂, —S(O)₂R, —S(O)₂NR₂, —S(O)R, —C(O)R, —C(O)OR, —C(O)NR₂, —C(O)NROR, —OC(O)R, —OC(O)NR₂, —NRC(O)OR, —NRC(O)R, —NRC(O)N(R)₂, and —NRS(O)₂R;
each R^{B} is independently an optionally substituted group selected from C₁₋₆ aliphatic, phenyl, a 3-12 membered saturated or partially unsaturated monocyclic, bicyclic, bridged bicyclic or spirocyclic carbocyclyl or heterocyclyl with 1-3 heteroatoms independently selected from nitrogen, oxygen, and sulfur, and a 5-6 membered heteroaryl ring having 1-4 heteroatoms independently selected from nitrogen, oxygen, and sulfur;
each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same nitrogen are optionally taken together with their intervening atoms to form a 4-7 membered saturated, partially unsaturated, or heteroaryl ring having 0-3 heteroatoms, in addition to the nitrogen atom to which they are attached, independently selected from nitrogen, oxygen, and sulfur;
p is independently 0 or 1; and
w, x, y, and z are independently 0, 1, 2, 3, or 4.

In some embodiments, the present invention provides a compound of formula I-11 or I-11′, wherein DBM is a deubiquitinase binding moiety thereby forming a compound of formula I-11-1:

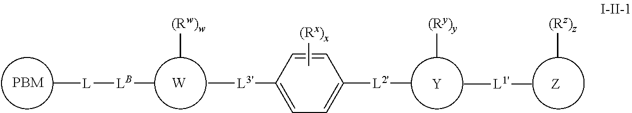

or a pharmaceutically acceptable salt thereof, wherein each of ring W, ring Y, ring Z, L^{B}, L^{3′}, L^{2′}, L^{1′}, R^{w}, R^{x}, R^{y}, R^{z}, w, x, y, z, L, and PBM is as defined above and described in embodiments herein, both singly and in combination.

In some embodiments, the present invention provides a compound of formula I-ll or I-ll′, wherein DBM is a deubiquitinase binding moiety thereby forming a compound of formula I-ll-2:

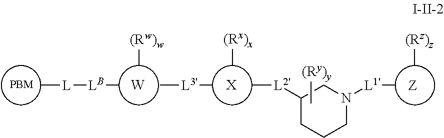

or a pharmaceutically acceptable salt thereof, wherein each of ring W, ring X, ring Z, L^{B}, L^{3′}, L^{2′}, L^{1′}, R^{w}, R^{x}, R^{y}, R^{z}, w, x, y, z, L, and PBM is as defined above and described in embodiments herein, both singly and in combination.

In some embodiments, the present invention provides a compound of formula I-11 or I-11′, wherein DBM is a deubiguitinase binding moiety thereby forming a compound of formula I-11-3:

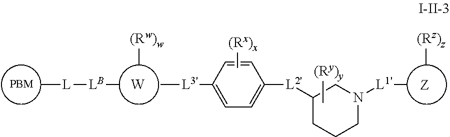

or a pharmaceutically acceptable salt thereof, wherein each of ring W, ring Z, L^{B}, L^{3′}, L^{2′}, L^{1′}, R^{w}, R^{x}, R^{y}, R^{z}, w, x, y, z, L, and PBM is as defined above and described in embodiments herein, both singly and in combination.

In some embodiments, the present invention provides a compound of formula I-ll or I-ll′, wherein DBM is a deubiquitinase binding moiety thereby forming a compound of formula I-ll-4:

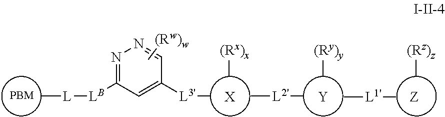

or a pharmaceutically acceptable salt thereof, wherein each of ring X, ring Y, ring Z, L^{B}, L^{3′}, L^{2′}, L^{1′}, R^{w}, R^{x}, R^{y}, R^{z}, w, x, y, z, L, and PBM is as defined above and described in embodiments herein, both singly and in combination.

In some embodiments, DBM is a covalent deubiquitinase binder. In some embodiments, DBM covalently binds OTUB1. In some embodiments, the DBM comprises a Michael accepter, e.g., α, β-unsaturated ketone which is capable of covalently binding to a deubiquitinase, e.g., OTUB1.

In some embodiments, the present invention provides a compound of formula I-aa, wherein DBM is a deubiquitinase binding moiety thereby forming a compound of formula I-mm:

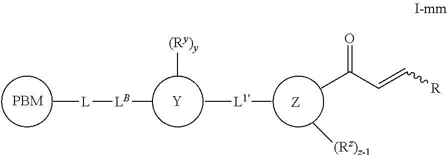

or a pharmaceutically acceptable salt thereof, wherein L and PBM are as defined above and described herein, and wherein:
Ring Z is a ring selected from phenylenyl, a 4-7 membered saturated or partially unsaturated carbocyclylenyl, a 4-7 membered saturated or partially unsaturated heterocyclylenyl with 1-3 heteroatoms independently selected from nitrogen, oxygen, and sulfur, and a 5-6 membered heteroarylenyl with 1-4 heteroatoms independently selected from nitrogen, oxygen and sulfur;
Ring Y is a ring selected from phenylenyl, a 4-7 membered saturated or partially unsaturated carbocyclylenyl, a 4-7 membered saturated or partially unsaturated heterocyclylenyl with 1-3 heteroatoms independently selected from nitrogen, oxygen, and sulfur, and a 5-6 membered heteroarylenyl with 1-4 heteroatoms independently selected from nitrogen, oxygen and sulfur;
each of L^{B}, and L^{1′}, is independently a covalent bond or a bivalent, saturated or partially unsaturated, straight or branched C₁₋₅ hydrocarbon chain, wherein 0-3 methylene units of L^{B}, or L^{1′}, are independently replaced by —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, or —OC(O)NR—, —NRC(O)O—;
each R^{y}, and R^{z} is independently selected from hydrogen, R^{B}, halogen, CN, —NO₂, —OR, —SR, —NR₂, —S(O)₂R, —S(O)₂NR₂, —S(O)R, —C(O)R, —C(O)OR, —C(O)NR₂, —C(O)NROR, —OC(O)R, —OC(O)NR₂, —NRC(O)OR, —NRC(O)R, —NRC(O)N(R)₂, and —NRS(O)₂R;
each R^{B} is independently an optionally substituted group selected from C₁₋₆ aliphatic, phenyl, a 3-12 membered saturated or partially unsaturated monocyclic, bicyclic, bridged bicyclic or spirocyclic carbocyclyl or heterocyclyl with 1-3 heteroatoms independently selected from nitrogen, oxygen, and sulfur, and a 5-6 membered heteroaryl ring having 1-4 heteroatoms independently selected from nitrogen, oxygen, and sulfur;
each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same nitrogen are optionally taken together with their intervening atoms to form a 4-7 membered saturated, partially unsaturated, or heteroaryl ring having 0-3 heteroatoms, in addition to the nitrogen atom to which they are attached, independently selected from nitrogen, oxygen, and sulfur; and
y, and z are independently 0, 1, 2, 3, or 4.

In some embodiments, the present invention provides a compound of formula I-mm, wherein DBM is a deubiquitinase binding moiety thereby forming a compound of formula I-mm-1:

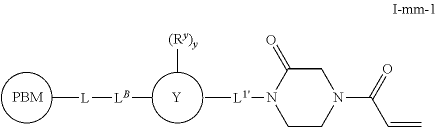

or a pharmaceutically acceptable salt thereof, wherein each of ring Y, L^{B}, L¹, R^{y}, y, L, and PBM is as defined above and described in embodiments herein, both singly and in combination.

In some embodiments, the present invention provides a compound of formula I-mm, wherein DBM is a deubiquitinase binding moiety thereby forming a compound of formula I-mm-2:

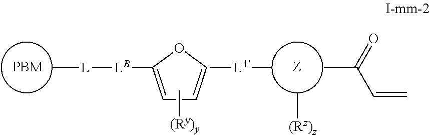

or a pharmaceutically acceptable salt thereof, wherein each of ring Z, L^{B}, L^{1′}, R^{y}, R^{z}, y, z, L, and PBM is as defined above and described in embodiments herein, both singly and in combination.

As defined above and described herein, ring Z is a ring selected from phenyl, a 4-7 membered saturated or partially unsaturated carbocyclic ring, an 8-10 membered bicyclic aromatic carbocyclic ring, a 4-8 membered saturated or partially unsaturated monocyclic heterocyclic ring having 1-2 heteroatoms independently selected from nitrogen, oxygen, or sulfur, a 5-6 membered monocyclic heteroaromatic ring having 1-4 heteroatoms independently selected from nitrogen, oxygen, or sulfur, and an 8-10 membered bicyclic heteroaromatic ring having 1-5 heteroatoms independently selected from nitrogen, oxygen, or sulfur.

As defined above and described herein, Ring Z is a ring selected from phenyl a 3-7 membered saturated or partially unsaturated monocyclic, bicyclic, bridged bicyclic or spirocyclic carbocyclic ring, a 3-7 membered saturated or partially unsaturated monocyclic, bicyclic, bridged bicyclic or spirocyclic heterocyclic ring with 1-3 heteroatoms independently selected from nitrogen, oxygen, and sulfur, and a 5-6 membered heteroaromatic ring with 1-4 heteroatoms independently selected from nitrogen, oxygen and sulfur. In some embodiments, ring Z is a 4-7 membered saturated or partially unsaturated carbocyclic ring. In some embodiments, ring Z is a phenyl. In some embodiments, ring Z is an 8-10 membered bicyclic aromatic carbocyclic ring. In some embodiments, ring Z is a 4-8 membered saturated or partially unsaturated monocyclic heterocyclic ring having 1-2 heteroatoms independently selected from nitrogen, oxygen, or sulfur. In some embodiments, ring Z is a 5-6 membered monocyclic heteroaromatic ring having 1-4 heteroatoms independently selected from nitrogen, oxygen, or sulfur. In some embodiments, ring Z is an 8-10 membered bicyclic heteroaromatic ring having 1-5 heteroatoms independently selected from nitrogen, oxygen, or sulfur.

In some embodiments, ring Z is a 3-7 membered saturated or partially unsaturated monocyclic, bicyclic, bridged bicyclic or spirocyclic carbocyclic ring. In some embodiments, ring Z is a 3-7 membered saturated or partially unsaturated monocyclic, bicyclic, bridged bicyclic or spirocyclic heterocyclic ring with 1-3 heteroatoms independently selected from nitrogen, oxygen, and sulfur. In some embodiments, ring Z is a 5-6 membered heteroaromatic ring with 1-4 heteroatoms independently selected from nitrogen, oxygen and sulfur.

In some embodiments, ring Z is cyclopropyl.

In some embodiments, ring Z is

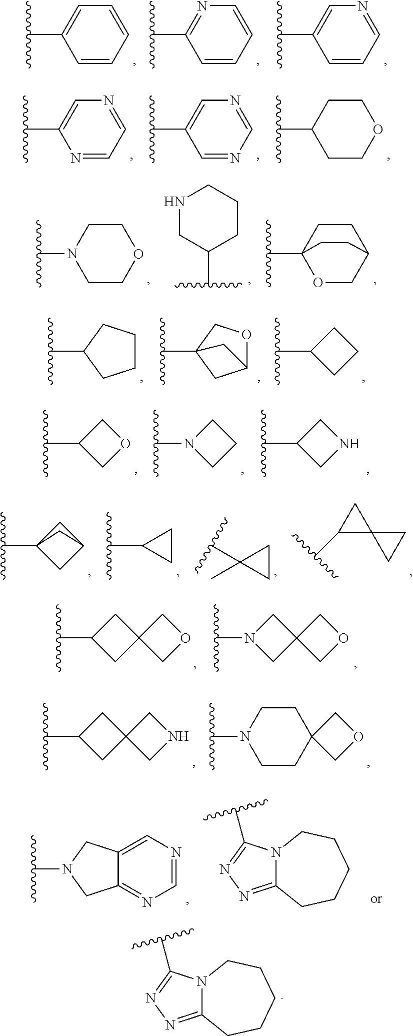

In some embodiments ring Z is

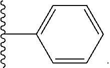 [SMILES: *c1ccccc1]

In some embodiments, ring Z is

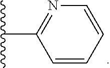 [SMILES: *c1ccccn1]

In some embodiments, ring Z is

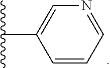 [SMILES: *c1cccnc1]

n some embodiments, ring Z is

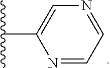 [SMILES: *c1cnccn1]

In some embodiments, ring Z is

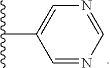 [SMILES: *c1cncnc1]

In some embodiments, ring Z is

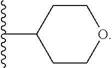 [SMILES: *C1CCOCC1]

In some embodiments, ring Z is

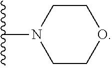 [SMILES: *N1CCOCC1]

In some embodiments, ring Z is

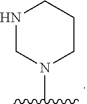 [SMILES: *N1CCCNC1]

In some embodiments, ring Z is

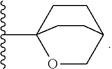 [SMILES: *C12CCC(CC1)CO2]

In some embodiments, ring Z is

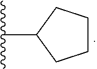 [SMILES: *C1CCCC1]

In some embodiments, ring Z is

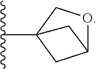 [SMILES: *C12COC(C1)C2]

In some embodiments, ring Z is

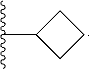 [SMILES: *C1CCC1]

In some embodiments, ring Z is

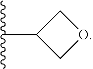 [SMILES: *C1COC1]

In some embodiments, ring Z is

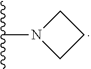 [SMILES: *N1CCC1]

In some embodiments, ring Z is

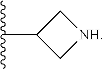 [SMILES: *C1CNC1]

In some embodiments, ring Z is

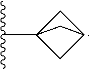 [SMILES: *C12CC(C1)C2]

In some embodiments, ring Z is

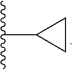 [SMILES: *C1CC1]

In some embodiments, A is

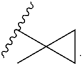 [SMILES: *C1(C)CC1]

In some embodiments, ring Z is

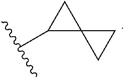 [SMILES: *C1CC12CC2]

In some embodiments, ring Z is

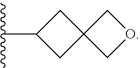 [SMILES: *C1CC2(COC2)C1]

In some embodiments, ring Z is

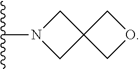 [SMILES: *N1CC2(COC2)C1]

In some embodiments, ring Z is

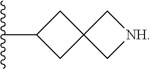 [SMILES: *C1CC2(CNC2)C1]

In some embodiments, ring Z is

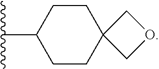 [SMILES: *C1CCC2(CC1)COC2]

In some embodiments, ring Z is

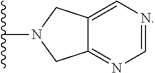 [SMILES: *N1Cc2cncnc2C1]

In some embodiments, ring Z is

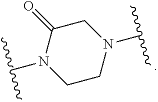 [SMILES: *N1CCN(*)C(=O)C1]

n some embodiments, ring Z is

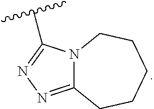 [SMILES: *c1nnc2n1CCCCC2]

In some embodiments, ring Z is selected from those depicted in the compounds of Table 1 below.

As defined above and described herein, ring Y is a ring selected from phenylenyl, a 4-7 membered saturated or partially unsaturated carbocyclylenyl, an 8-10 membered bicyclic aromatic carbocyclylenyl, a 4-8 membered saturated or partially unsaturated monocyclic heterocyclylenyl having 1-2 heteroatoms independently selected from nitrogen, oxygen, or sulfur, a 5-6 membered monocyclic heteroarylenyl having 1-4 heteroatoms independently selected from nitrogen, oxygen, or sulfur, and an 8-10 membered bicyclic heteroarylenyl having 1-5 heteroatoms independently selected from nitrogen, oxygen, or sulfur.

In some embodiments, ring Y is a phenylenyl. In some embodiments, ring Y is a 4-7 membered saturated or partially unsaturated carbocyclylenyl. In some embodiments, ring Y is an 8-10 membered bicyclic aromatic carbocyclylenyl. In some embodiments, ring Y is a 4-8 membered saturated or partially unsaturated monocyclic heterocyclylenyl having 1-2 heteroatoms independently selected from nitrogen, oxygen, or sulfur. In some embodiments, ring Y is a 5-6 membered monocyclic heteroarylenyl having 1-4 heteroatoms independently selected from nitrogen, oxygen, or sulfur. In some embodiments, ring Y is an 8-10 membered bicyclic heteroarylenyl having 1-5 heteroatoms independently selected from nitrogen, oxygen, or sulfur.

In some embodiments, ring Y is

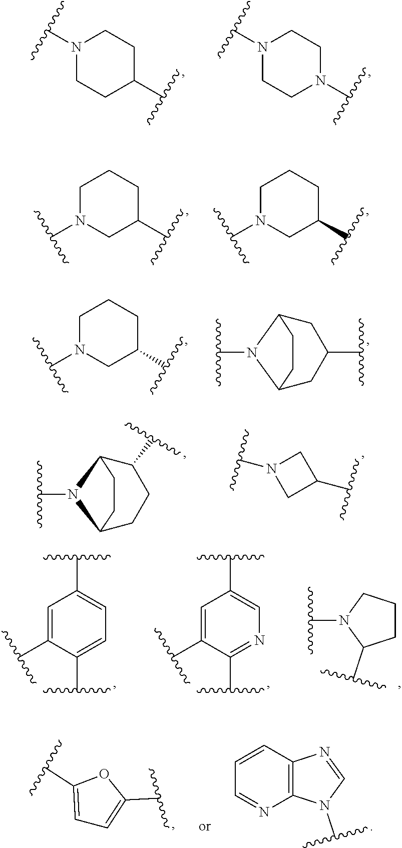

In some embodiments, ring Y is

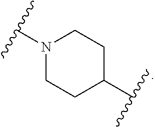 [SMILES: *C1CCN(*)CC1]

In some embodiments, ring Y is

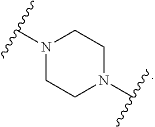 [SMILES: *N1CCN(*)CC1]

In some embodiments, ring Y is

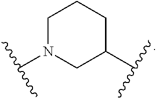 [SMILES: *C1CCCN(*)C1]

In some embodiments, ring Y is

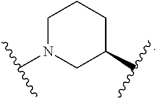 [SMILES: *[C@@H]1CCCN(*)C1]

In some embodiments, ring Y is

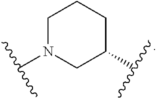 [SMILES: *[C@H]1CCCN(*)C1]

In some embodiments, ring Y is

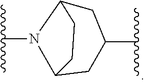 [SMILES: *C1CC2CCC(C1)N2*]

In some embodiments, ring Y is

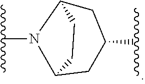 [SMILES: *[C@H]1C[C@H]2CC[C@@H](C1)N2*]

In some embodiments, ring Y is

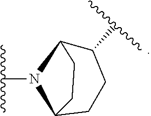 [SMILES: *[C@@H]1CC[C@@H]2CC[C@H]1N2*]

In some embodiments, ring Y is

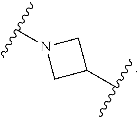 [SMILES: *C1CN(*)C1]

In some embodiments, ring Y is

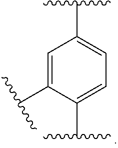 [SMILES: *c1ccc(*)c(*)c1]

In some embodiments, ring Y is

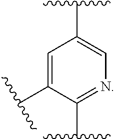 [SMILES: *c1cnc(*)c(*)c1]

In some embodiments, ring Y is

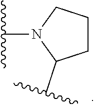 [SMILES: *C1CCCN1*]

In some embodiments, ring Y is

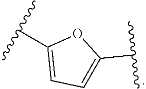 [SMILES: *c1ccc(*)o1]

In some embodiments, ring Y is

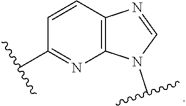 [SMILES: *c1ccc2ncn(*)c2n1]

In some embodiments, ring Y is selected from those depicted in the compounds of Table 1 below.

As defined above and described herein, ring X is a ring selected from phenylenyl, a 4-7 membered saturated or partially unsaturated carbocyclylenyl, a 4-7 membered saturated or partially unsaturated heterocyclylenyl with 1-3 heteroatoms independently selected from nitrogen, oxygen, and sulfur, and a 5-6 membered heteroarylenyl with 1-4 heteroatoms independently selected from nitrogen, oxygen and sulfur.

In some embodiments, ring X is a phenylenyl. In some embodiments, ring X is a 4-7 membered saturated or partially unsaturated carbocyclylenyl. In some embodiments, ring X is a 4-7 membered saturated or partially unsaturated heterocyclylenyl with 1-3 heteroatoms independently selected from nitrogen, oxygen, and sulfur. In some embodiments, ring X is and a 5-6 membered heteroarylenyl with 1-4 heteroatoms independently selected from nitrogen, oxygen and sulfur.

In some embodiments, ring X is

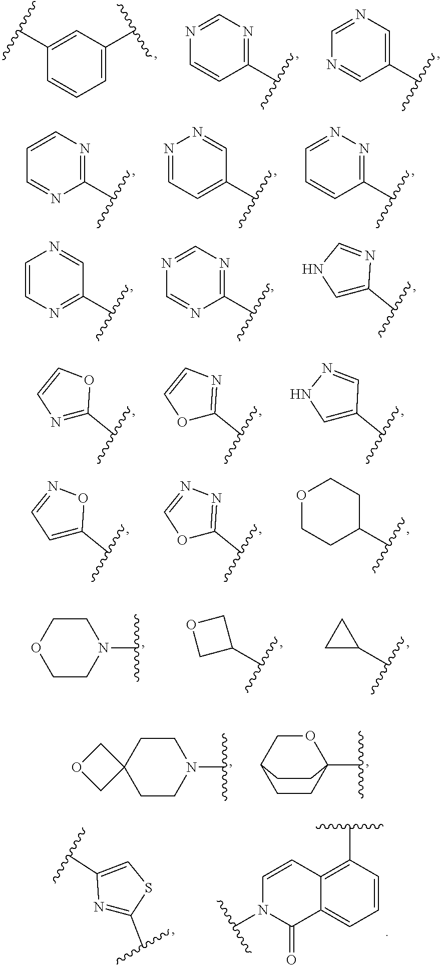

In some embodiments ring X is

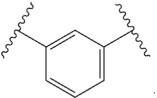 [SMILES: *c1cccc(*)c1]

In some embodiments, ring X is

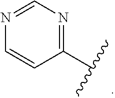 [SMILES: *c1ccncn1]

In some embodiments, ring X is

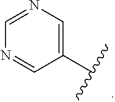 [SMILES: *c1cncnc1]

In some embodiments, ring X is

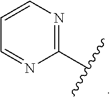 [SMILES: *c1ncccn1]

In some embodiments, ring X is

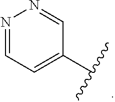 [SMILES: *c1ccnnc1]

In some embodiments, ring X is

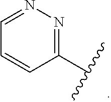 [SMILES: *c1cccnn1]

In some embodiments, ring X is

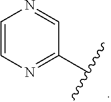 [SMILES: *c1cnccn1]

In some embodiments, ring X is

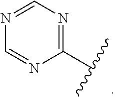 [SMILES: *c1ncncn1]

In some embodiments, ring X is

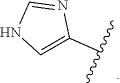 [SMILES: *c1c[nH]cn1]

In some embodiments, ring X is

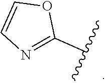 [SMILES: *c1ncco1]

In some embodiments, ring X is

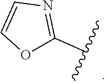 [SMILES: *c1ncco1]

In some embodiments, ring X is

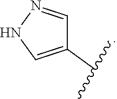 [SMILES: *c1cn[nH]c1]

In some embodiments, ring X is

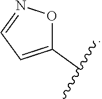 [SMILES: *c1ccno1]

In some embodiments, ring X is

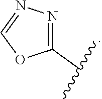 [SMILES: *c1nnco1]

In some embodiments, ring X is

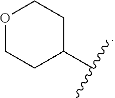 [SMILES: *C1CCOCC1]

In some embodiments, ring X is

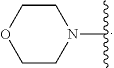 [SMILES: *N1CCOCC1]

In some embodiments, ring X is

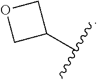 [SMILES: *C1COC1]

In some embodiments, ring X is

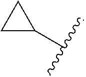 [SMILES: *C1CC1]

In some embodiments, ring X is

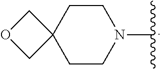 [SMILES: *N1CCC2(CC1)COC2]

In some embodiments, ring X is

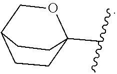 [SMILES: *C12CCC(CC1)CO2]

In some embodiments, ring X is

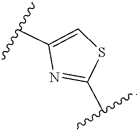 [SMILES: *c1csc(*)n1]

. In some embodiments, ring X is

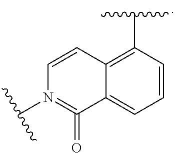

In some embodiments, ring X is selected from those depicted in the compounds of Table 1 below.

As defined above and described herein, ring W is a ring selected from phenylenyl, a 4-7 membered saturated or partially unsaturated carbocyclylenyl, a 4-7 membered saturated or partially unsaturated heterocyclylenyl with 1-3 heteroatoms independently selected from nitrogen, oxygen, and sulfur, and a 5-6 membered heteroarylenyl with 1-4 heteroatoms independently selected from nitrogen, oxygen and sulfur.

In some embodiments, ring W is a phenylenyl. In some embodiments, ring W is a 4-7 membered saturated or partially unsaturated carbocyclylenyl. In some embodiments, ring W is a 4-7 membered saturated or partially unsaturated heterocyclylenyl with 1-3 heteroatoms independently selected from nitrogen, oxygen, and sulfur. In some embodiments, ring W is and a 5-6 membered heteroarylenyl with 1-4 heteroatoms independently selected from nitrogen, oxygen and sulfur.

In some embodiments, ring W is

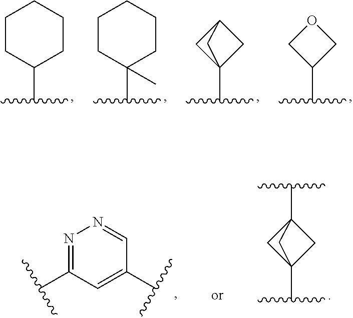

In some embodiments, ring W is

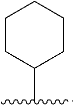 [SMILES: *C1CCCCC1]

In some embodiments, B is

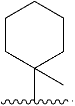 [SMILES: *C1(C)CCCCC1]

In some embodiments, ring W is

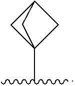 [SMILES: *C12CC(C1)C2]

In some embodiments, ring W is

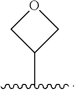 [SMILES: *C1COC1]

In some embodiments ring W is

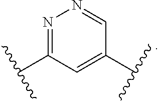 [SMILES: *c1cnnc(*)c1]

In some embodiments, ring W is

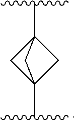 [SMILES: *C12CC(*)(C1)C2]

In some embodiments, ring W is selected from those depicted in the compounds of Table 1 below.

As defined above and described herein, L is a covalent bond or a bivalent, saturated or partially unsaturated, straight or branched C₁₋₅ hydrocarbon chain, wherein 0-3 methylene units of L^{1′} are independently replaced by —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, or —OC(O)NR—, —NRC(O)O—.

In some embodiments, L^{1′} is a covalent bond. In some embodiments, L^{1′} is a bivalent, saturated or partially unsaturated, straight or branched C₁₋₅ hydrocarbon chain, wherein 0-3 methylene units of L^{1′} are independently replaced by —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, or —OC(O)NR—, —NRC(O)O—.

In some embodiments, L^{1′} is —O—. In some embodiments, L^{1′} is —NR—. In some embodiments, L^{1′} is —S—. In some embodiments, L^{1′} is —OC(O)—. In some embodiments, L^{1′} is —C(O)O—. In some embodiments, L^{1′} is —C(O)—. In some embodiments, L^{1′} is —S(O)—. In some embodiments, L^{1′} is —S(O)₂—. In some embodiments, L^{1′} is —NRS(O)₂—. In some embodiments, L^{1′} is —S(O)₂NR—. In some embodiments, L^{1′} is —NRC(O)—. In some embodiments, L^{1′} is —C(O)NR—. In some embodiments, L^{1′} is —OC(O)NR—. In some embodiments, L^{1′} is —NRC(O)O—.

In some embodiments, L^{1′}, is —CH₂—.

In some embodiments, L^{1′} is

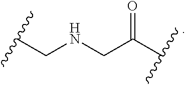 [SMILES: *CNCC(*)=O]

In some embodiments, L^{1′} is

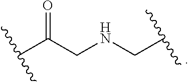 [SMILES: *CNCC(*)=O]

In some embodiments, L^{1′} is

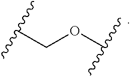 [SMILES: *CO*]

In some embodiments, L^{1′} is

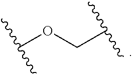 [SMILES: *CO*]

In some embodiments, L^{1′} is

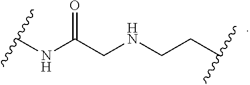 [SMILES: *CCNCC(=O)N*]

In some embodiments, L^{1′} is

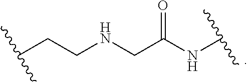 [SMILES: *CCNCC(=O)N*]

In some embodiments, L^{1′} is

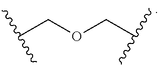 [SMILES: *COC*]

In some embodiments, L^{1′} is

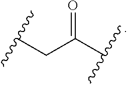 [SMILES: *CC(*)=O]

In some embodiments, L^{1′} is

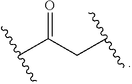 [SMILES: *CC(*)=O]

In some embodiments, L^{1′} is

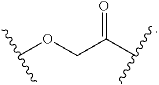 [SMILES: *OCC(*)=O]

In some embodiments, L^{1′} is

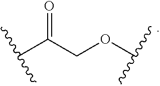 [SMILES: *OCC(*)=O]

In some embodiments, L^{1′} is

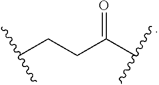 [SMILES: *CCC(*)=O]

In some embodiments, L^{1′} is

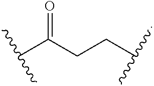

In some embodiments, L^{1′} is

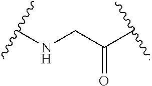

In some embodiments, L^{1′} is

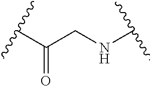

In some embodiments, L^{1′} is

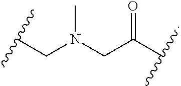

In some embodiments, L^{1′} is

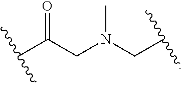

. In some embodiments, L^{1′} is

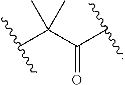

In some embodiments, L^{1′} is

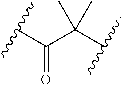

In some embodiments, L^{1′} is selected from those depicted in the compounds of Table 1 below.

As defined above and described herein, L^{2′} is a covalent bond or a bivalent, saturated or partially unsaturated, straight or branched C₁₋₅ hydrocarbon chain, wherein 0-3 methylene units of L^{2′} are independently replaced by —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, or —OC(O)NR—, —NRC(O)O—.

In some embodiments, L^{2′} is a covalent bond. In some embodiments, L^{2′} is a bivalent, saturated or partially unsaturated, straight or branched C₁₋₅ hydrocarbon chain, wherein 0-3 methylene units of L^{2′} are independently replaced by —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, or —OC(O)NR—, —NRC(O)O—.

In some embodiments, L^{2′} is —O—. In some embodiments, L^{2′} is —NR—. In some embodiments, L^{2′} is —S—. In some embodiments, L^{2′} is —OC(O)—. In some embodiments, L^{2′} is —C(O)O—. In some embodiments, L^{2′} is —C(O)—. In some embodiments, L^{2′} is —S(O)—. In some embodiments, L^{2′} is —S(O)₂—. In some embodiments, L^{2′} is —NRS(O)₂—. In some embodiments, L^{2′} is —S(O)₂NR—. In some embodiments, L^{2′} is —NRC(O)—. In some embodiments, L^{2′} is —C(O)NR—. In some embodiments, L^{2′} is or —OC(O)NR—. In some embodiments, L^{2′} is —NRC(O)O—. In some embodiments, L^{2′} is

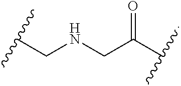

In some embodiments, L^{2′} is

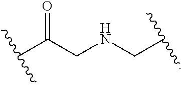

In some embodiments, L^{2′} is

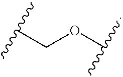

In some embodiments, L^{2′} is

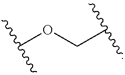

In some embodiments, L^{2′} is

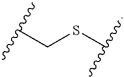

In some embodiments, L^{2′} is

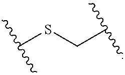

In some embodiments, L^{2′} is

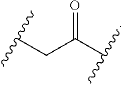

In some embodiments, L^{2′} is

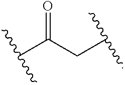

In some embodiments, L^{2′} is

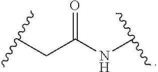 [SMILES: *CC(=O)N*]

In some embodiments, L^{2′} is

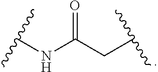 [SMILES: *CC(=O)N*]

In some embodiments, L^{2′} is

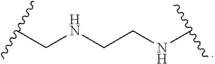 [SMILES: *CNCCN*]

In some embodiments, L^{2′} is

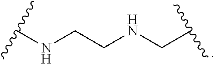 [SMILES: *CNCCN*]

In some embodiments, L^{2′} is

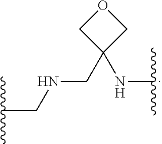 [SMILES: *CNCC1(N*)COC1]

In some embodiments, L^{2′} is

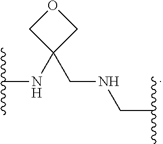 [SMILES: *CNCC1(N*)COC1]

In some embodiments, L^{2′} is

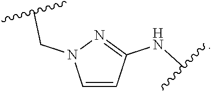 [SMILES: *Cn1ccc(N*)n1]

In some embodiments, L^{2′} is

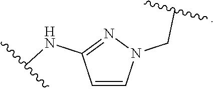 [SMILES: *Cn1ccc(N*)n1]

In some embodiments, L^{2′} is

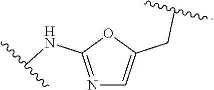 [SMILES: *Cc1cnc(N*)o1]

In some embodiments, L^{2′} is selected from those depicted in the compounds of Table 1 below.

As defined above and described herein, L^{3′}, is a covalent bond or a bivalent, saturated or partially unsaturated, straight or branched C₁₋₅ hydrocarbon chain, wherein 0-3 methylene units of L^{3′} are independently replaced by —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, or —OC(O)NR—, —NRC(O)O—.

In some embodiments, L^{3′}, is a covalent bond. In some embodiments, L^{3′}, is a bivalent, saturated or partially unsaturated, straight or branched C₁₋₅ hydrocarbon chain, wherein 0-3 methylene units of L³ are independently replaced by —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, or —OC(O)NR—, —NRC(O)O—.

In some embodiments, L^{3′}, is —O—. In some embodiments, L^{3′}, is —NR—. In some embodiments, L^{3′}, is —S—. In some embodiments, L^{3′}, is —OC(O)—. In some embodiments, L^{3′}, is —C(O)O—. In some embodiments, L^{3′}, is —C(O)—. In some embodiments, L^{3′}, is —S(O)—. In some embodiments, L^{3′}, is —S(O)₂—. In some embodiments, L^{3′}, is —NRS(O)₃—. In some embodiments, L^{3′}, is —S(O)₂NR—. In some embodiments, L^{3′}, is —NRC(O)—. In some embodiments, L^{3′}, is —C(O)NR—. In some embodiments, L^{3′}, is or —OC(O)NR—.

In some embodiments, L^{3′}, is —NRC(O)O—.

In some embodiments, L^{3′}, is —C—.

In some embodiments, L^{3′} is selected from those depicted in the compounds of Table 1 below.

As defined above and described herein, L^{B}, is a covalent bond or a bivalent, saturated or partially unsaturated, straight or branched C₁₋₅ hydrocarbon chain, wherein 0-3 methylene units of L^{B} are independently replaced by —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, or —OC(O)NR—, —NRC(O)O—.

In some embodiments, L^{B} is a covalent bond. In some embodiments, L^{B} is a bivalent, saturated or partially unsaturated, straight or branched C₁₋₅ hydrocarbon chain, wherein 0-3 methylene units of L^{B} are independently replaced by —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, or —OC(O)NR—, —NRC(O)O—.

In some embodiments, L^{B} is —O—. In some embodiments, L^{B} is —NR—. In some embodiments, L^{B} is —S—. In some embodiments, L^{B} is —OC(O)—. In some embodiments, L^{B} is —C(O)O—. In some embodiments, L^{B} is —C(O)—. In some embodiments, L^{B} is —S(O)—. In some embodiments, L^{B} is —S(O)₂—. In some embodiments, L^{B} is —NRS(O)₃—. In some embodiments, L^{B} is —S(O)₂NR—. In some embodiments, L^{B} is —NRC(O)—. In some embodiments, L^{B} is —C(O)NR—. In some embodiments, L^{B} is or —OC(O)NR—. In some embodiments, L^{B} is —NRC(O)O—.

In some embodiments, L^{B} is —C—. In some embodiments, L^{B} is

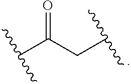 [SMILES: *CC(*)=O]

In some embodiments, L^{B} is

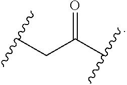 [SMILES: *CC(*)=O]

In some embodiments, L^{B} is

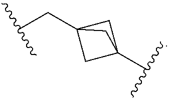 [SMILES: *CC12CC(*)(C1)C2]

In some embodiments, L^{B} is

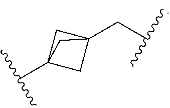 [SMILES: *CC12CC(*)(C1)C2]

In some embodiments, L^{B} is

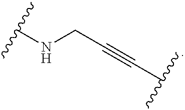 [SMILES: *C#CCN*]

In some embodiments, L^{B} is

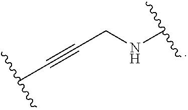 [SMILES: *C#CCN*]

In some embodiments, L^{B} is

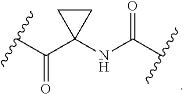 [SMILES: *C(=O)NC1(C(*)=O)CC1]

In some embodiments, L^{B} is

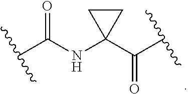 [SMILES: *C(=O)NC1(C(*)=O)CC1]

In some embodiments, L^{B} is selected from those depicted in the compounds of Table 1 below.

As defined above and described herein, each R^{w} is independently selected from hydrogen, R^{B}, halogen, —CN, —NO₂, —OR, —SR, —NR₂, —S(O)₂R, —S(O)₂NR₂, —S(O)R, —C(O)R, —C(O)OR, —C(O)NR₂, —C(O)NROR, —OC(O)R, —OC(O)NR₂, —NRC(O)OR, —NRC(O)R, —NRC(O)N(R)₂, and —NRS(O)₂R.

In some embodiments, one or more of R^{w} is hydrogen. In some embodiments, one or more of R^{w} is R^{B}. In some embodiments, one or more of R^{w} is halogen. In some embodiments, one or more of R^{w} is —CN. In some embodiments, one or more of R^{w} is —NO₂. In some embodiments, one or more of R^{w} is —OR. In some embodiments, one or more of R^{w} is —SR. In some embodiments, one or more of R^{w} is —NR₂. In some embodiments, one or more of R^{w} is —S(O)₂R. In some embodiments, one or more of R^{w} is —S(O)₂NR₂. In some embodiments, one or more of R^{w} is —S(O)R. In some embodiments, one or more of R^{w} is —C(O)R. In some embodiments, one or more of R^{w} is —C(O)OR. In some embodiments, one or more of R^{w} is —C(O)NR₂. In some embodiments, one or more of R^{w} is —C(O)NROR. In some embodiments, one or more of R^{w} is —OC(O)R. In some embodiments, one or more of R^{w} is —OC(O)NR₂. In some embodiments, one or more of R^{w} is —NRC(O)OR. In some embodiments, one or more of R^{w} is —NRC(O)R. In some embodiments, one or more of R^{w} is —NRC(O)N(R)₂. In some embodiments, one or more of R^{w} is —NRS(O)₂R.

In some embodiments, is

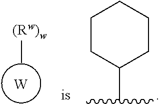

In some embodiments, R^{w} is selected from those depicted in the compounds of Table 1 below.

As defined above and described herein, each R^{x} is independently selected from hydrogen, R^{B}, halogen, —CN, —NO₂, —OR, —SR, —NR₂, —S(O)₂R, —S(O)₂NR₂, —S(O)R, —C(O)R, —C(O)OR, —C(O)NR₂, —C(O)NROR, —OC(O)R, —OC(O)NR₂, —NRC(O)OR, —NRC(O)R, —NRC(O)N(R)₂, and —NRS(O)₂R.

In some embodiments, one or more of R^{x} is hydrogen. In some embodiments, one or more of R^{x} is R^{B}. In some embodiments, one or more of R^{x} is halogen. In some embodiments, one or more of R^{x} is —CN. In some embodiments, one or more of R^{x} is —NO₂. In some embodiments, one or more of R^{x} is —OR. In some embodiments, one or more of R^{x} is —SR. In some embodiments, one or more of R^{x} is —NR₂. In some embodiments, one or more of R^{x} is —S(O)₂R. In some embodiments, one or more of R^{x} is —S(O)₂NR₂ In some embodiments, one or more of R^{x} is —S(O)R. In some embodiments, one or more of R^{x} is —C(O)R. In some embodiments, one or more of R^{x} is —C(O)OR. In some embodiments, one or more of R^{x} is —C(O)NR₂. In some embodiments, one or more of R^{x} is —C(O)NROR. In some embodiments, one or more of R is —OC(O)R. In some embodiments, one or more of R^{x} is —OC(O)NR₂. In some embodiments, one or more of R^{x} is —NRC(O)OR. In some embodiments, one or more of R is —NRC(O)R. In some embodiments, one or more of R^{x} is —NRC(O)N(R)₂. In some embodiments, one or more of R is —NRS(O)₂R.

In some embodiments is

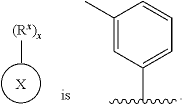

In some embodiments,

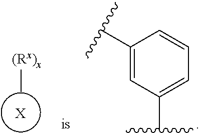

In some embodiments,

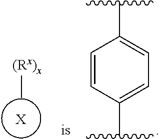

In some embodiments,

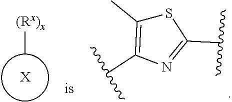

. In some embodiments,

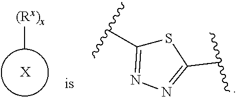

In some embodiments,

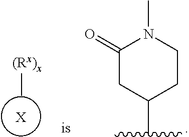

In some embodiments, is

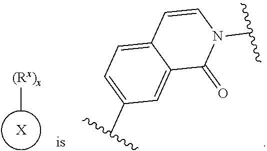

In some embodiments,

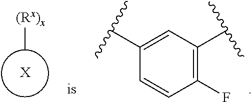

In some embodiments,

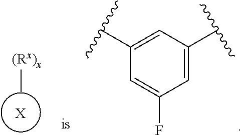

In some embodiments,

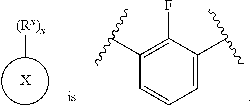

In some embodiments is

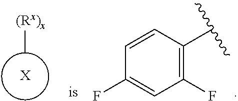

In some embodiments,

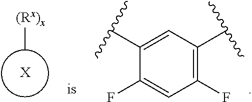

In some embodiments, is

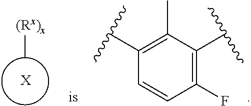

In some embodiments,

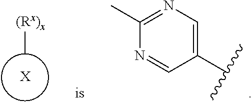

In some embodiments,

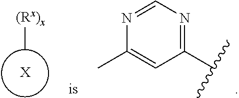

In some embodiments,

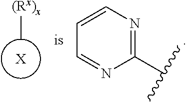

In some embodiments,

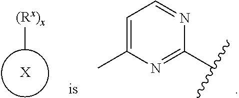

. In some embodiments,

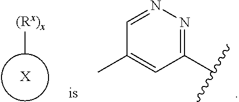

In some embodiments,

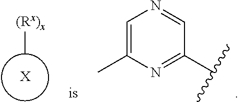

In some embodiments,

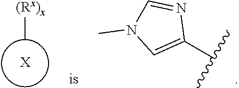

In some embodiments,

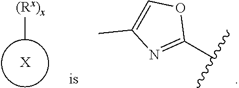

In some embodiments,

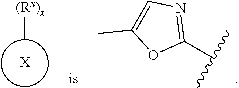

In some embodiments,

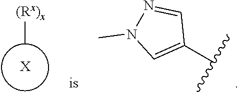

. In some embodiments,

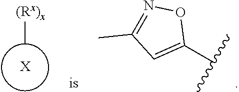

In some embodiments,

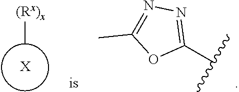

In some embodiments, is

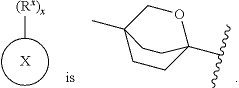

In some embodiments, is

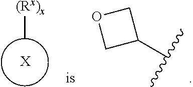

In some embodiments, is

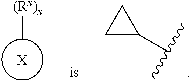

In some embodiments,

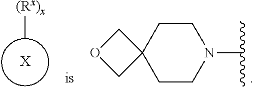

In some embodiments, R is selected from those depicted in the compounds of Table 1 below.

As defined above and described herein, each R^{y} is independently selected from hydrogen, R^{B}, halogen, —CN, —NO₂, —OR, —SR, —NR₂, —S(O)₂R, —S(O)₂NR₂, —S(O)R, —C(O)R, —C(O)OR, —C(O)NR₂, —C(O)NROR, —OC(O)R, —OC(O)NR₂, —NRC(O)OR, —NRC(O)R, —NRC(O)N(R)₂, and —NRS(O)₂R.

In some embodiments, one or more of R^{y} is hydrogen. In some embodiments, one or more of R^{y} is R^{B}. In some embodiments, one or more of R^{y} is halogen. In some embodiments, one or more of R^{y} is —CN. In some embodiments, one or more of R^{y} is —NO₂. In some embodiments, one or more of R^{y} is —OR. In some embodiments, one or more of R^{y} is —SR. In some embodiments, one or more of R^{y} is —NR₂. In some embodiments, one or more of R^{y} is —S(O)₂R. In some embodiments, one or more of R^{y} is —S(O)₂NR₂ In some embodiments, one or more of R^{y} is —S(O)R. In some embodiments, one or more of R^{y} is —C(O)R. In some embodiments, one or more of R^{y} is —C(O)OR. In some embodiments, one or more of R^{y} is —C(O)NR₂. In some embodiments, one or more of R^{y} is —C(O)NROR. In some embodiments, one or more of R^{y} is —OC(O)R. In some embodiments, one or more of R^{y} is —OC(O)NR₂. In some embodiments, one or more of R^{y} is —NRC(O)OR. In some embodiments, one or more of R^{y} is —NRC(O)R. In some embodiments, one or more of R^{y} is —NRC(O)N(R)₂. In some embodiments, one or more of R^{y} is —NRS(O)₂R.

In some embodiments, one or more of R^{y} is fluoro.

In some embodiments, is

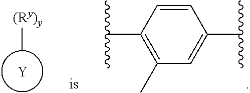

In some embodiments,

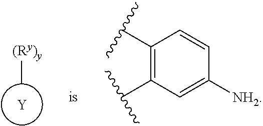

In some embodiments,

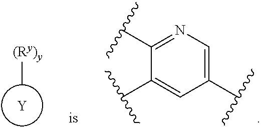

In some embodiments,

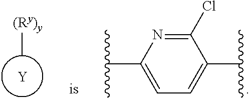

In some embodiments,

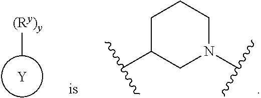

In some embodiments,

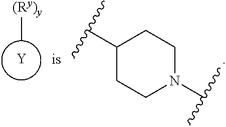

In some embodiments,

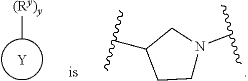

. In some embodiments,

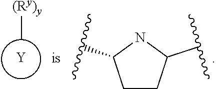

In some embodiments,

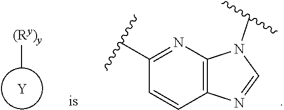

In some embodiments,

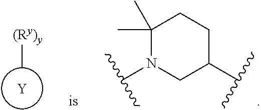

In some embodiments,

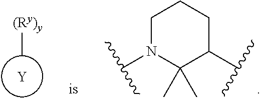

In some embodiments,

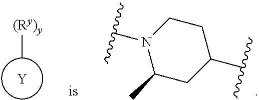

In some embodiments,

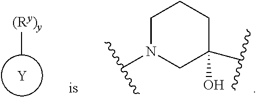

In some embodiments,

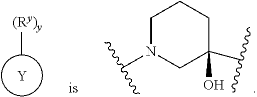

In some embodiments,

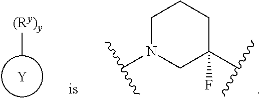

In some embodiments,

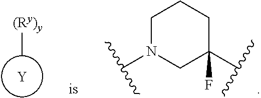

In some embodiments, R is selected from those depicted in the compounds of Table 1 below.

As defined above and described herein, each R^{z} is independently selected from hydrogen, R^{B}, halogen, —CN, —NO₂, —OR, —SR, —NR₂, —S(O)₂R, —S(O)₂NR₂, —S(O)R, —C(O)R, —C(O)OR, —C(O)NR₂, —C(O)NROR, —OC(O)R, —OC(O)NR₂, —NRC(O)OR, —NRC(O)R, —NRC(O)N(R)₂, and —NRS(O)₂R.

In some embodiments, one or more of R^{z} is hydrogen. In some embodiments, one or more of R^{z} is R^{B}. In some embodiments, one or more of R^{z} is halogen. In some embodiments, one or more of R^{z} is —CN. In some embodiments, one or more of R^{z} is —NO₂. In some embodiments, one or more of R^{z} is —OR. In some embodiments, one or more of R^{z} is —SR. In some embodiments, one or more of R^{z} is —NR₂. In some embodiments, one or more of R^{z} is —S(O)₂R. In some embodiments, one or more of R^{z} is —S(O)₂NR₂ In some embodiments, one or more of R^{z} is —S(O)R. In some embodiments, one or more of R^{z} is —C(O)R. In some embodiments, one or more of R^{z} is —C(O)OR. In some embodiments, one or more of R^{z} is —C(O)NR₂. In some embodiments, one or more of R^{z} is —C(O)NROR. In some embodiments, one or more of R^{z} is —OC(O)R. In some embodiments, one or more of R^{z} is —OC(O)NR₂. In some embodiments, one or more of R^{z} is —NRC(O)OR. In some embodiments, one or more of R^{z} is —NRC(O)R. In some embodiments, one or more of R^{z} is —NRC(O)N(R)₂. In some embodiments, one or more of R^{z} is —NRS(O)₂R.

In some embodiments, one or more of R^{z} is methyl. In some embodiments, one or more of R^{z} is ethyl.

In some embodiments,

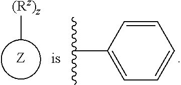

In some embodiments,

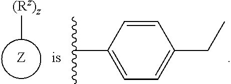

In some embodiments,

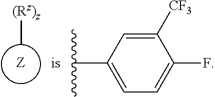

In some embodiments,

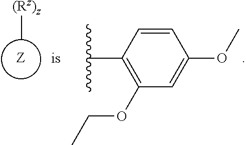

In some embodiments,

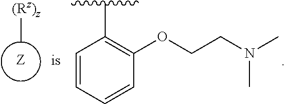

In some embodiments,

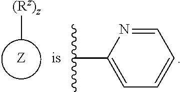

In some embodiments,

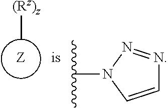

In some embodiments,

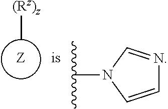

In some embodiments,

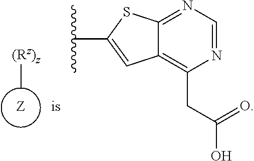

In some embodiments,

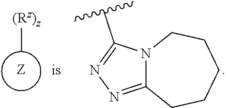

In some embodiments,

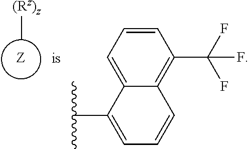

In some embodiments,

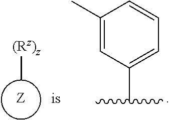

In some embodiments,

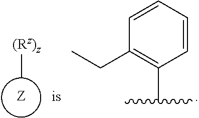

In some embodiments,

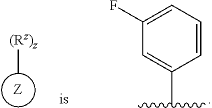

In some embodiments,

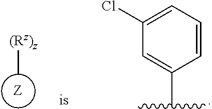

In some embodiments

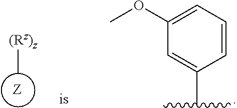

In some embodiments,

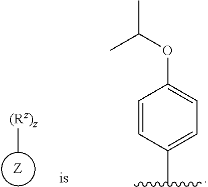

In some embodiments,

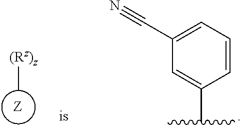

In some embodiments,

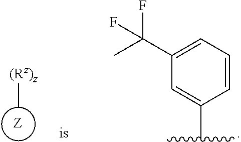

In some embodiments,

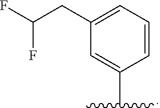 [SMILES: *c1cccc(CC(F)F)c1]

In some embodiments,

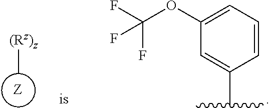

In some embodiments,

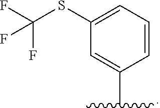 [SMILES: *c1cccc(SC(F)(F)F)c1]

In some embodiments,

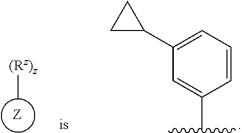

In some embodiments,

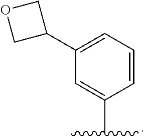 [SMILES: *c1cccc(C2COC2)c1]

In some embodiments,

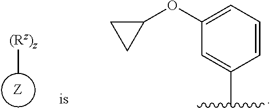

In some embodiments,

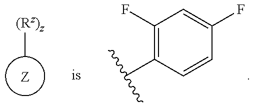

In some embodiments,

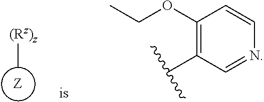

In some embodiments,

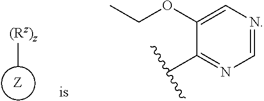

In some embodiments,

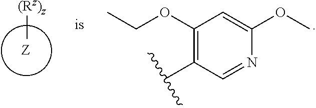

In some embodiments,

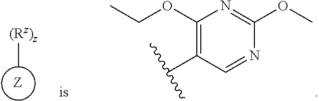

In some embodiments

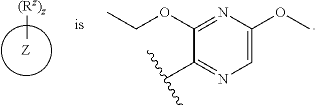

In some embodiments,

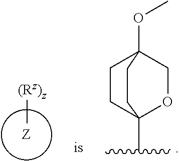

In some embodiments,

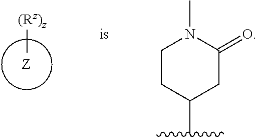

In some embodiments,

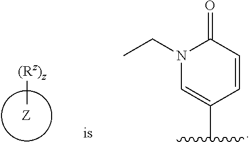

In some embodiments,

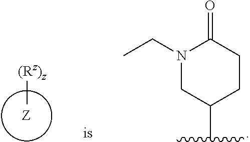

In some embodiments,

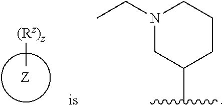

In some embodiments,

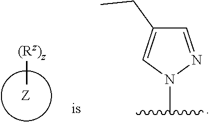

In some embodiments,

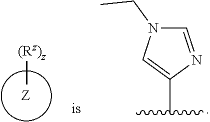

In some embodiments,

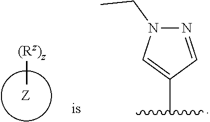

In some embodiments,

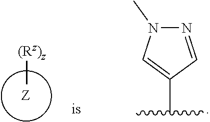

In some embodiments,

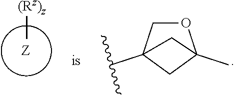

In some embodiments,

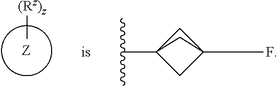

In some embodiments is

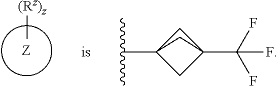

In some embodiments,

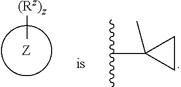

In some embodiments,

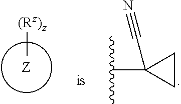

In some embodiments,

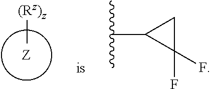

In some embodiments,

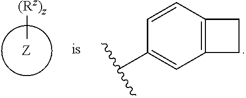

In some embodiments;

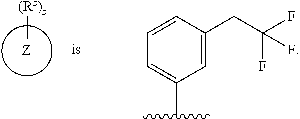

In some embodiments, R^{z} is selected from those depicted in the compounds of Table 1 below.

As defined above and described herein, each R^{B} is independently an optionally substituted group selected from C₁₋₆ aliphatic, phenyl, a 3-12 membered saturated or partially unsaturated monocyclic, bicyclic, bridged bicyclic or spirocyclic carbocyclyl or heterocyclyl with 1-3 heteroatoms independently selected from nitrogen, oxygen, and sulfur, and a 5-6 membered heteroaryl ring having 1-4 heteroatoms independently selected from nitrogen, oxygen, and sulfur.

In some embodiments, of R_{B} is an optionally substituted C₁₋₆ aliphatic. In some embodiments, R^{B} is a phenyl. In some embodiments, R^{B} is a 3-12 membered saturated or partially unsaturated monocyclic. In some embodiments, R^{B} is an optionally substituted bicyclic. In some embodiments, of R^{B} is an optionally substituted bridged bicyclic or spirocyclic carbocyclyl. In some embodiments, R^{B} is a heterocyclyl with 1-3 heteroatoms independently selected from nitrogen, oxygen, and sulfur. In some embodiments, R^{B} is an optionally substituted 5-6 membered heteroaryl ring having 1-4 heteroatoms independently selected from nitrogen, oxygen, and sulfur.

In some embodiments, R^{B} is selected from those depicted in the compounds of Table 1 below.

As defined above and described herein, each -Cy′- is independently an optionally substituted bivalent ring selected from phenylenyl, an 8-10 membered bicyclic arylenyl, a 3-11 membered saturated or partially unsaturated monocyclic, bicyclic, bridged bicyclic or spirocyclic carbocyclylenyl, a 3-11 membered saturated or partially unsaturated monocyclic, bicyclic, bridged bicyclic or spirocyclic heterocyclylenyl having 1-3 heteroatoms independently selected from nitrogen, oxygen, and sulfur, a 5-6 membered heteroarylenyl having 1-4 heteroatoms independently selected from nitrogen, oxygen, and sulfur, and an 8-10 membered bicyclic heteroarylenyl having 1-5 heteroatoms independently selected from nitrogen, oxygen, and sulfur.

In some embodiments, one or more -Cy′- is an optionally substituted phenylenyl. In some embodiments, one or more -Cy′- is an optionally substituted 8-10 membered bicyclic arylenyl. In some embodiments, one or more -Cy′- is an optionally substituted 3-11 membered saturated or partially unsaturated monocyclic, bicyclic, bridged bicyclic or spirocyclic carbocyclylenyl. In some embodiments, one or more -Cy′- is an optionally substituted 3-11 membered saturated or partially unsaturated monocyclic, bicyclic, bridged bicyclic or spirocyclic heterocyclylenyl having 1-3 heteroatoms independently selected from nitrogen, oxygen, and sulfur. In some embodiments, one or more -Cy′- is an optionally substituted 5-6 membered heteroarylenyl having 1-4 heteroatoms independently selected from nitrogen, oxygen, and sulfur. In some embodiments, one or more -Cy′- is an optionally substituted 8-10 membered bicyclic heteroarylenyl having 1-5 heteroatoms independently selected from nitrogen, oxygen, and sulfur.

In some embodiments, -Cy′- is selected from those depicted in the compounds of Table 1 below.

As defined above and described herein, each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or two R groups on the same nitrogen are optionally taken together with their intervening atoms to form a 4-7 membered saturated, partially unsaturated, or heteroaryl ring having 0-3 heteroatoms, in addition to the nitrogen atom to which they are attached, independently selected from nitrogen, oxygen, and sulfur.

In some embodiments, one or more of R is hydrogen. In some embodiments, one or more of R is a C₁₋₆ aliphatic. In some embodiments, one or more of R is phenyl. In some embodiments, one or more of R is a 4-7 membered saturated or partially unsaturated heterocyclic having 1-2 heteroatoms independently selected from nitrogen, oxygen and sulfur. In some embodiments, one or more of R is and a 5-6 membered heteroaryl ring having 1-4 heteroatoms independently selected from nitrogen, oxygen, and sulfur. In some embodiments, two R groups on the same nitrogen are optionally taken together with their intervening atoms to form a 4-7 membered saturated, partially unsaturated, or heteroaryl ring having 0-3 heteroatoms, in addition to the nitrogen atom to which they are attached, independently selected from nitrogen, oxygen, and sulfur.

In some embodiments, R is selected from those depicted in the compounds of Table 1 below.

As defined above and described herein, m is independently 0 or 1. In some embodiments, m is 0. In some embodiments, m is 1.

In some embodiments, m is selected from those depicted in the compounds of Table 1 below.

As defined above and described herein, n is independently 0 or 1. In some embodiments, n is 0. In some embodiments, n is 1.

In some embodiments, n is selected from those depicted in the compounds of Table 1 below.

As defined above and described herein, o is independently 0 or 1. In some embodiments, o is 0. In some embodiments, o is 1.

In some embodiments, o is selected from those depicted in the compounds of Table 1 below.

As defined above and described herein, w is 0, 1, 2, 3, or 4. In some embodiments, w is 0. In some embodiments, w is 1. In some embodiments, w is 2. In some embodiments, w is 3. In some embodiments, w is 4.

In some embodiments, w is selected from those depicted in the compounds of Table 1 below.

As defined above and described herein, x is 0, 1, 2, 3, or 4. In some embodiments, x is 0. In some embodiments, x is 1. In some embodiments, x is 2. In some embodiments, x is 3. In some embodiments, x is 4.

In some embodiments, x is selected from those depicted in the compounds of Table 1 below.

As defined above and described herein, y is 0, 1, 2, 3, or 4. In some embodiments, y is 0. In some embodiments, y is 1. In some embodiments, y is 2. In some embodiments, y is 3. In some embodiments, y is 4.

In some embodiments, y is selected from those depicted in the compounds of Table 1 below.

As defined above and described herein, z is 0, 1, 2, 3, or 4. In some embodiments, z is 0. In some embodiments, z is 1. In some embodiments, z is 2. In some embodiments, z is 3. In some embodiments, z is 4.

In some embodiments, z is selected from those depicted in the compounds of Table 1 below.

In some embodiments, DBM is

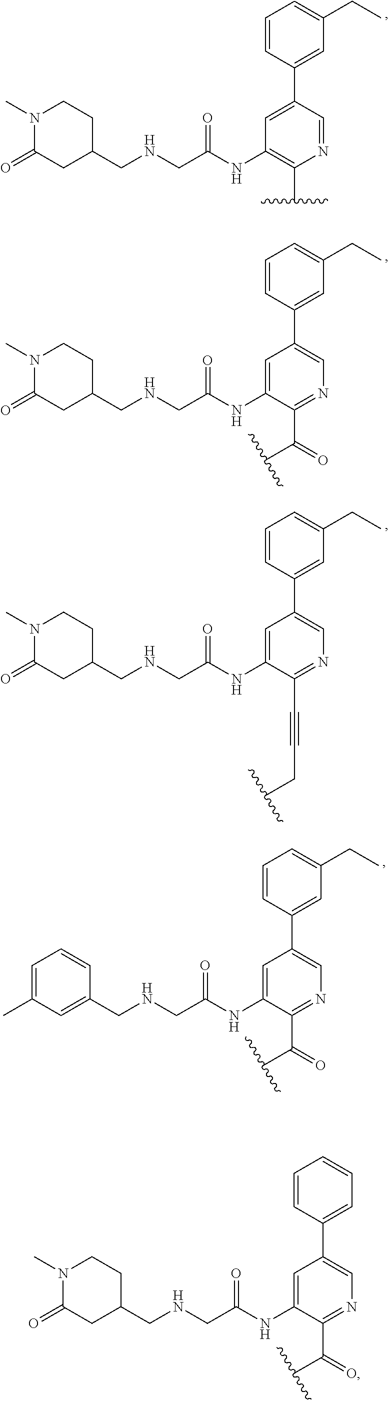

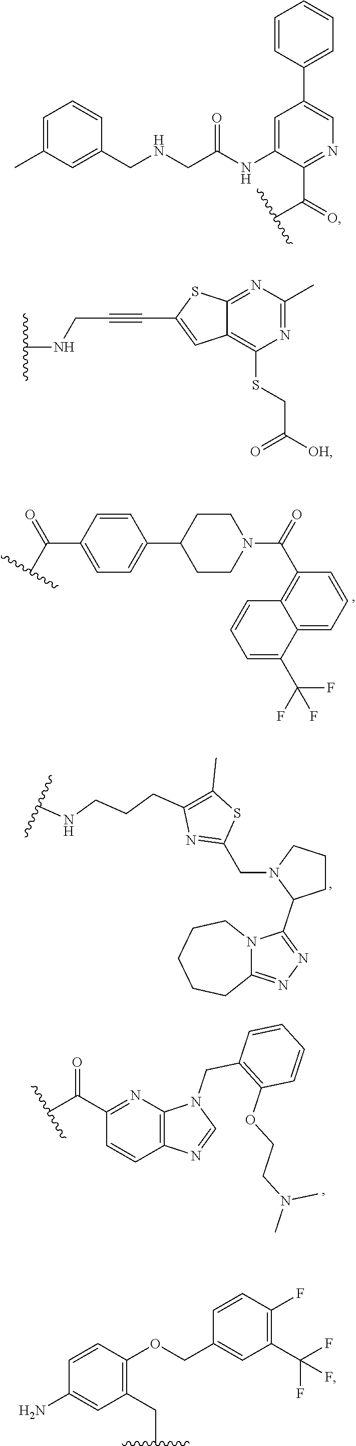

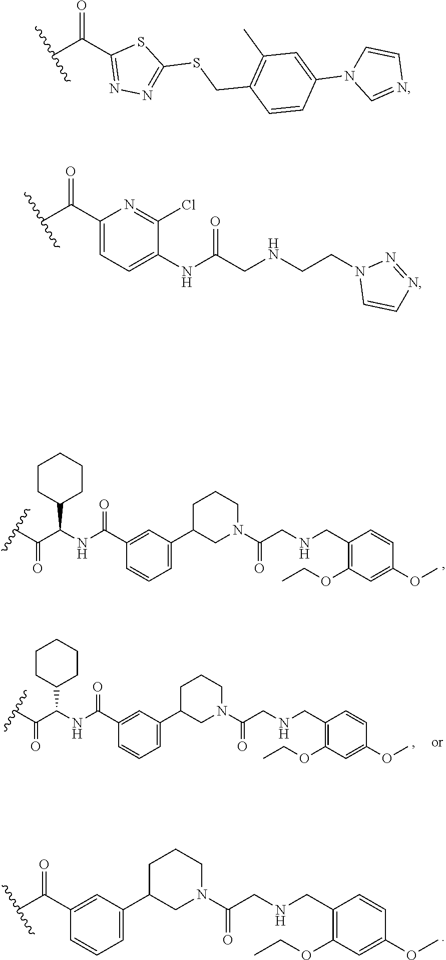 [SMILES: *C(=O)c1nnc(SCc2ccc(-n3ccnc3)cc2C)s1 ; *C(=O)c1ccc(NC(=O)CNCCn2ccnn2)c(Cl)n1 ; *C(=O)[C@H](NC(=O)c1cccc(C2CCCN(C(=O)CNCc3ccc(OC)cc3OCC)C2)c1)C1CCCCC1 ; *C(=O)[C@@H](NC(=O)c1cccc(C2CCCN(C(=O)CNCc3ccc(OC)cc3OCC)C2)c1)C1CCCCC1 ; *C(=O)c1cccc(C2CCCN(C(=O)CNCc3ccc(OC)cc3OCC)C2)c1]

In some embodiments, DBM is

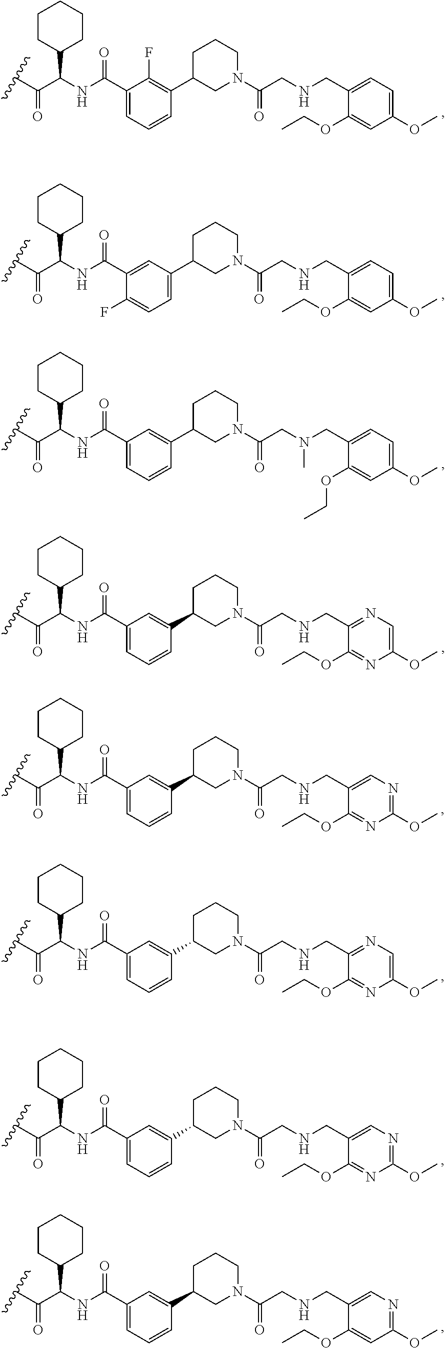

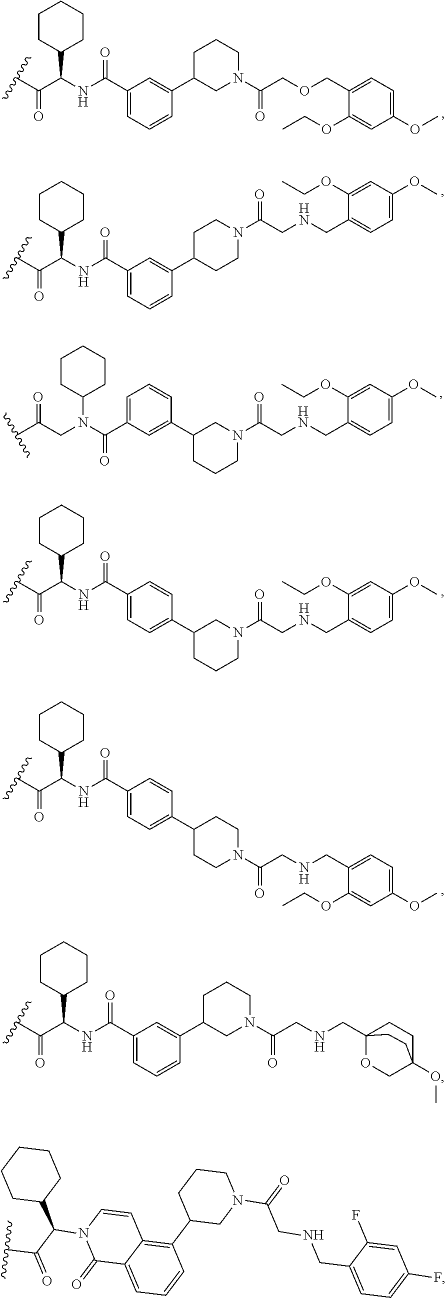

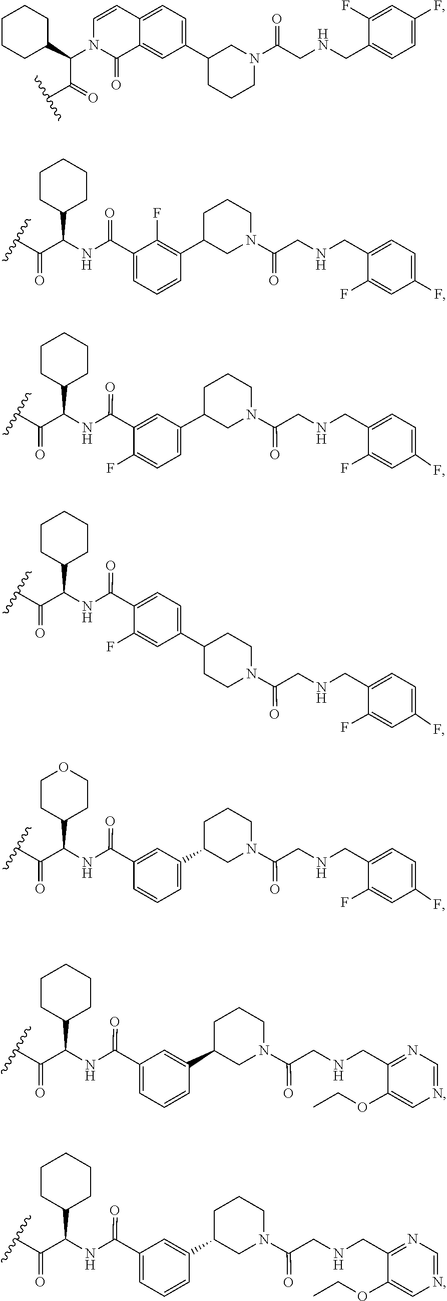

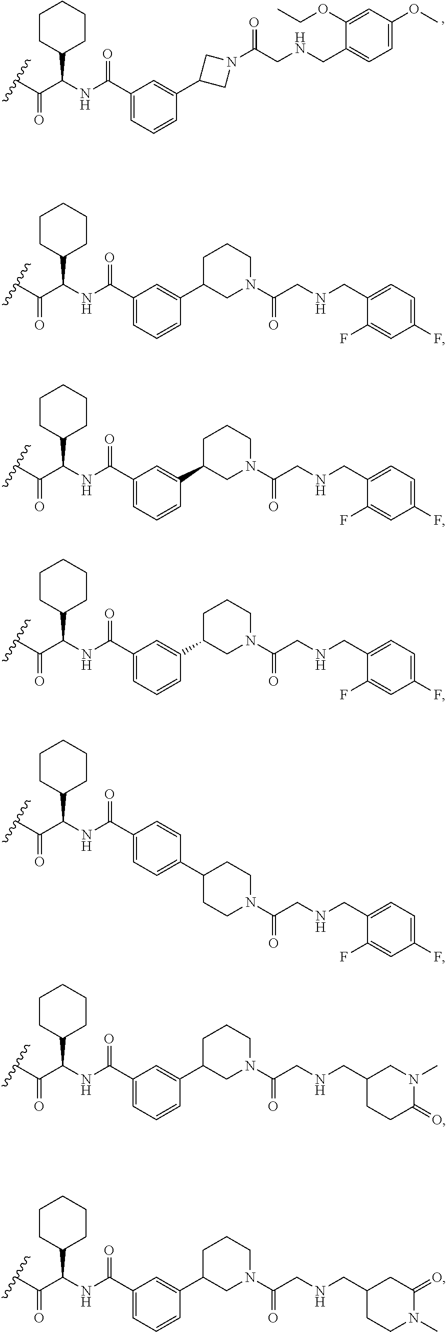

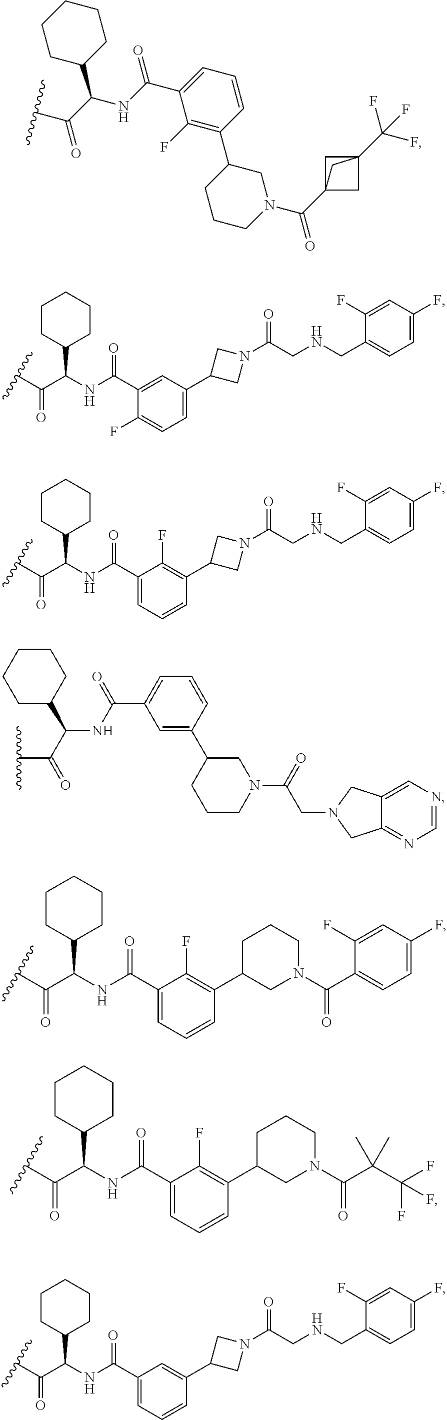

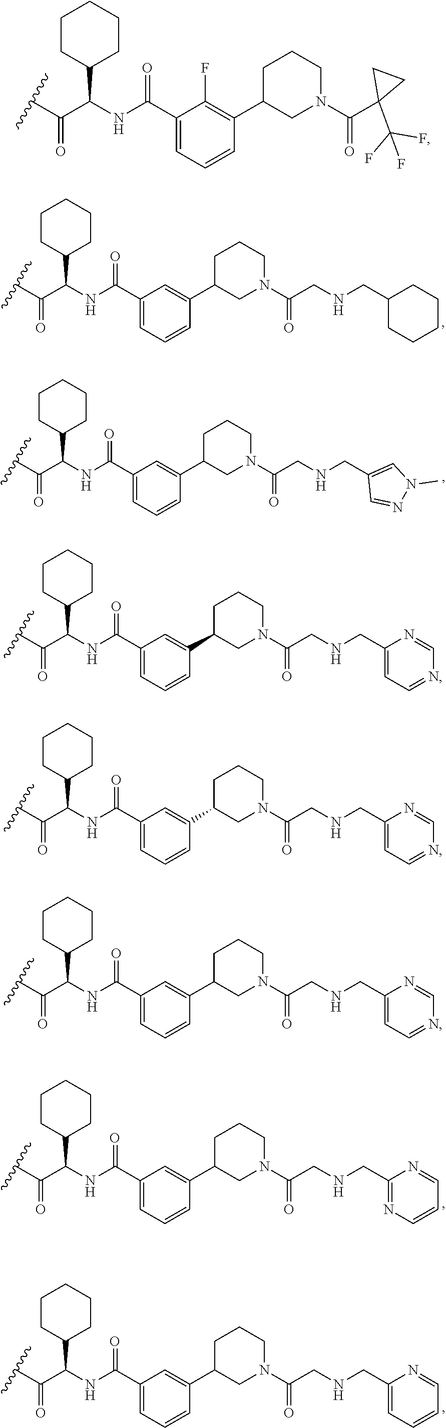

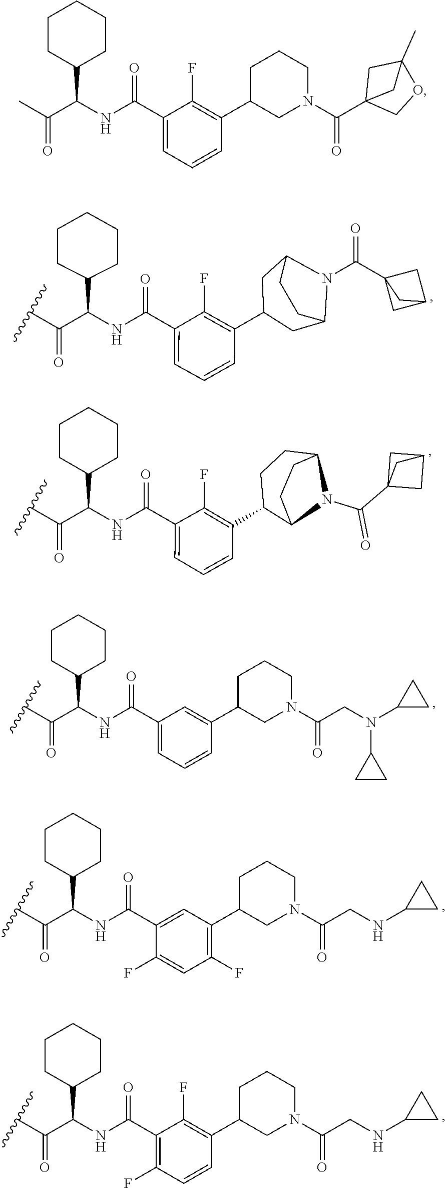 [SMILES: CC(=O)[C@H](NC(=O)c1cccc(C2CCCN(C(=O)C34COC(C)(C3)C4)C2)c1F)C1CCCCC1 ; *C(=O)[C@H](NC(=O)c1cccc(C2CC3CCC(C2)N3C(=O)C23CC(C2)C3)c1F)C1CCCCC1 ; *C(=O)[C@H](NC(=O)c1cccc([C@@H]2CC[C@@H]3CC[C@H]2N3C(=O)C23CC(C2)C3)c1F)C1CCCCC1 ; *C(=O)[C@H](NC(=O)c1cccc(C2CCCN(C(=O)CN(C3CC3)C3CC3)C2)c1)C1CCCCC1 ; *C(=O)[C@H](NC(=O)c1cc(C2CCCN(C(=O)CNC3CC3)C2)c(F)cc1F)C1CCCCC1 ; *C(=O)[C@H](NC(=O)c1c(F)ccc(C2CCCN(C(=O)CNC3CC3)C2)c1F)C1CCCCC1]

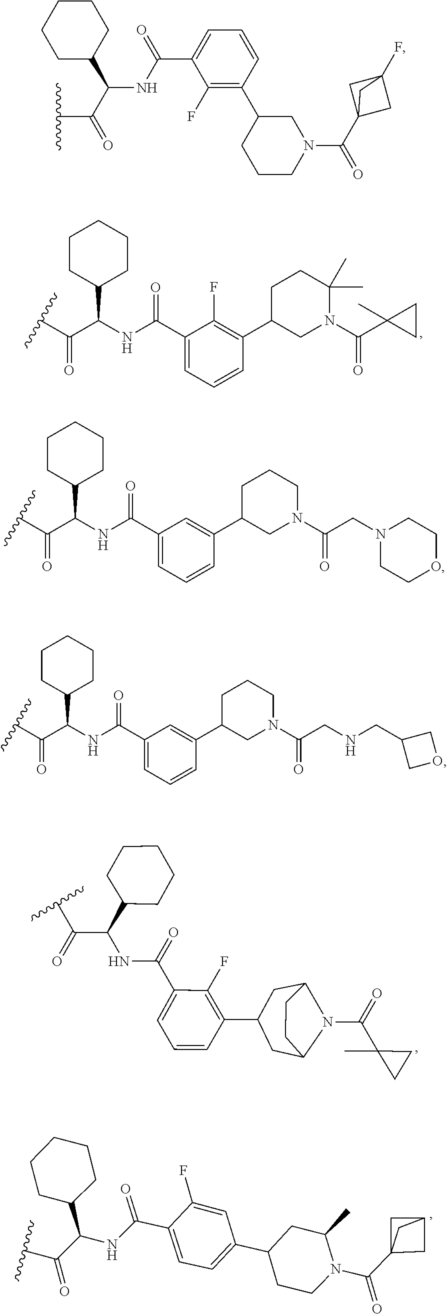 [SMILES: *C(=O)[C@H](NC(=O)c1cccc(C2CCCN(C(=O)C34CC(F)(C3)C4)C2)c1F)C1CCCCC1 ; *C(=O)[C@H](NC(=O)c1cccc(C2CCC(C)(C)N(C(=O)C3(C)CC3)C2)c1F)C1CCCCC1 ; *C(=O)[C@H](NC(=O)c1cccc(C2CCCN(C(=O)CN3CCOCC3)C2)c1)C1CCCCC1 ; *C(=O)[C@H](NC(=O)c1cccc(C2CCCN(C(=O)CNCC3COC3)C2)c1)C1CCCCC1 ; *C(=O)[C@H](NC(=O)c1cccc(C2CC3CCC(C2)N3C(=O)C2(C)CC2)c1F)C1CCCCC1 ; *C(=O)[C@H](NC(=O)c1ccc(C2CCN(C(=O)C34CC(C3)C4)[C@H](C)C2)cc1F)C1CCCCC1]

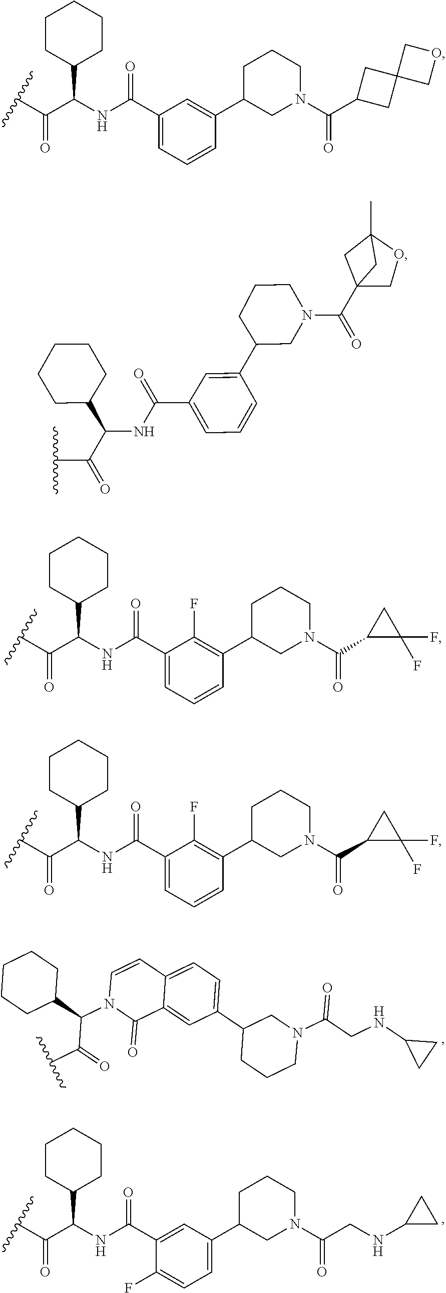 [SMILES: *C(=O)[C@H](NC(=O)c1cccc(C2CCCN(C(=O)C3CC4(COC4)C3)C2)c1)C1CCCCC1 ; *C(=O)[C@H](NC(=O)c1cccc(C2CCCN(C(=O)C34COC(C)(C3)C4)C2)c1)C1CCCCC1 ; *C(=O)[C@H](NC(=O)c1cccc(C2CCCN(C(=O)[C@@H]3CC3(F)F)C2)c1F)C1CCCCC1 ; *C(=O)[C@H](NC(=O)c1cccc(C2CCCN(C(=O)[C@H]3CC3(F)F)C2)c1F)C1CCCCC1 ; *C(=O)[C@@H](C1CCCCC1)n1ccc2ccc(C3CCCN(C(=O)CNC4CC4)C3)cc2c1=O ; *C(=O)[C@H](NC(=O)c1cc(C2CCCN(C(=O)CNC3CC3)C2)ccc1F)C1CCCCC1]

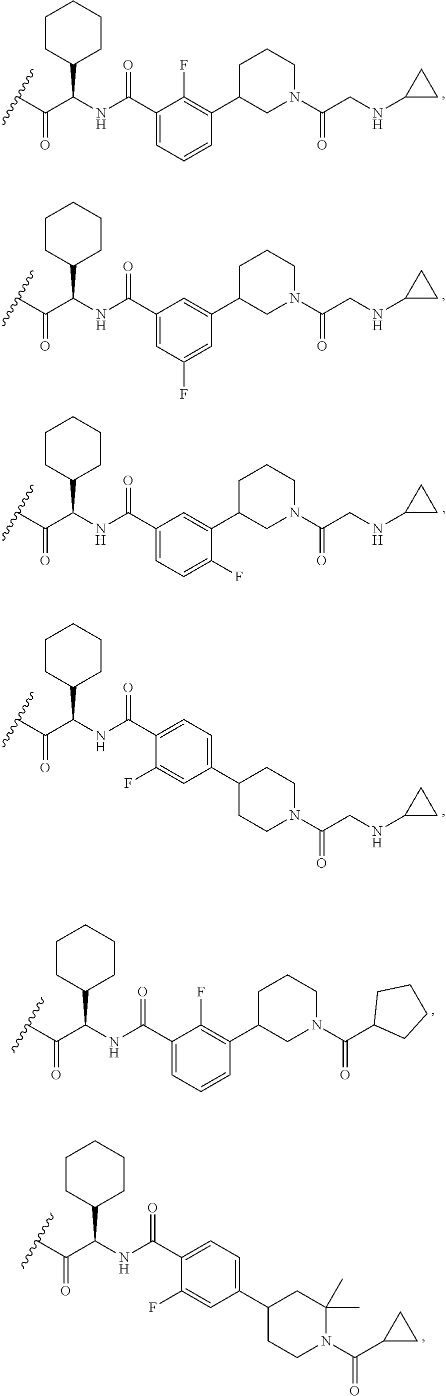 [SMILES: *C(=O)[C@H](NC(=O)c1cccc(C2CCCN(C(=O)CNC3CC3)C2)c1F)C1CCCCC1 ; *C(=O)[C@H](NC(=O)c1cc(F)cc(C2CCCN(C(=O)CNC3CC3)C2)c1)C1CCCCC1 ; *C(=O)[C@H](NC(=O)c1ccc(F)c(C2CCCN(C(=O)CNC3CC3)C2)c1)C1CCCCC1 ; *C(=O)[C@H](NC(=O)c1ccc(C2CCN(C(=O)CNC3CC3)CC2)cc1F)C1CCCCC1 ; *C(=O)[C@H](NC(=O)c1cccc(C2CCCN(C(=O)C3CCCC3)C2)c1F)C1CCCCC1 ; *C(=O)[C@H](NC(=O)c1ccc(C2CCN(C(=O)C3CC3)C(C)(C)C2)cc1F)C1CCCCC1]

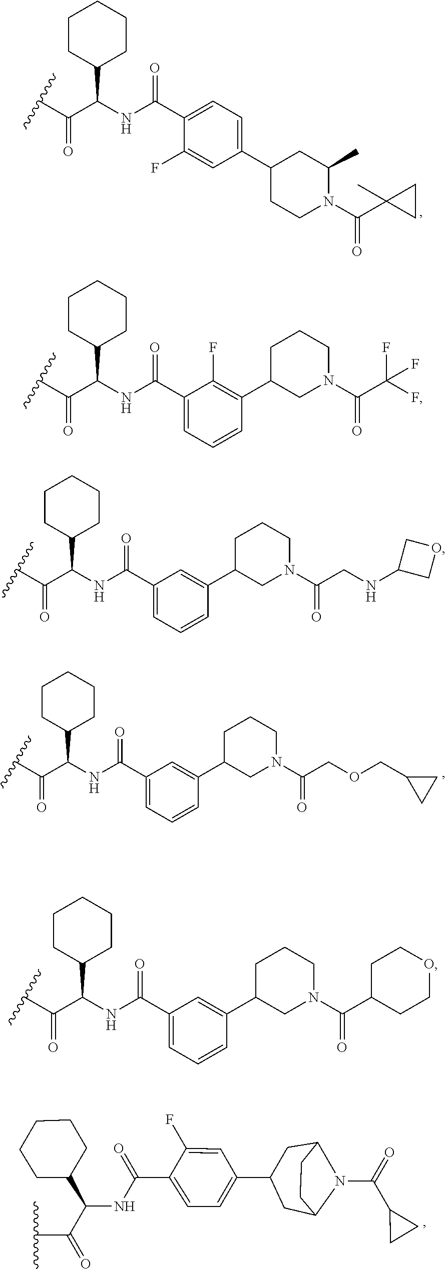

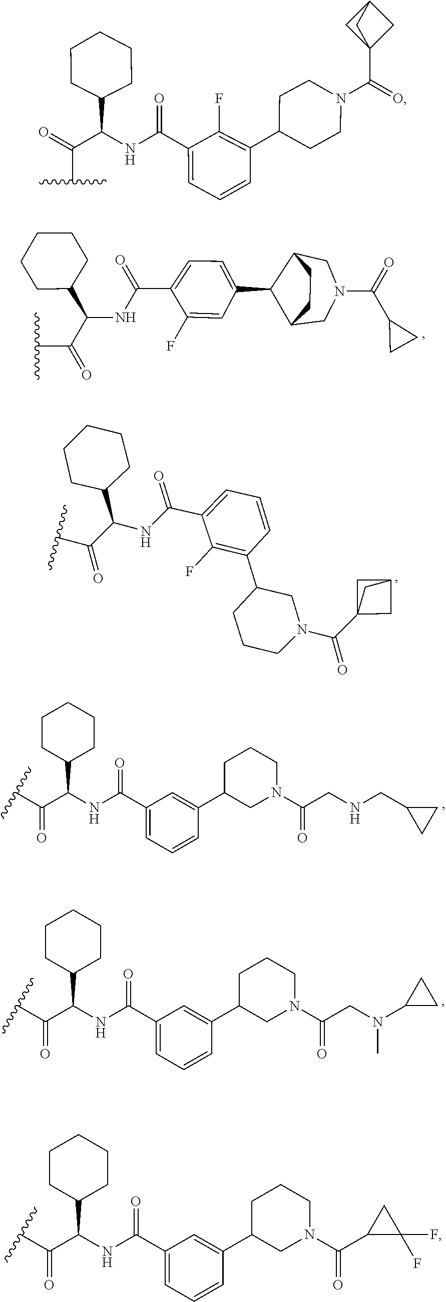 [SMILES: *C(=O)[C@H](NC(=O)c1cccc(C2CCN(C(=O)C34CC(C3)C4)CC2)c1F)C1CCCCC1 ; *C(=O)[C@H](NC(=O)c1ccc([C@@H]2[C@@H]3CC[C@H]2CN(C(=O)C2CC2)C3)cc1F)C1CCCCC1 ; *C(=O)[C@H](NC(=O)c1cccc(C2CCCN(C(=O)C34CC(C3)C4)C2)c1F)C1CCCCC1 ; *C(=O)[C@H](NC(=O)c1cccc(C2CCCN(C(=O)CNCC3CC3)C2)c1)C1CCCCC1 ; *C(=O)[C@H](NC(=O)c1cccc(C2CCCN(C(=O)CN(C)C3CC3)C2)c1)C1CCCCC1 ; *C(=O)[C@H](NC(=O)c1cccc(C2CCCN(C(=O)C3CC3(F)F)C2)c1)C1CCCCC1]

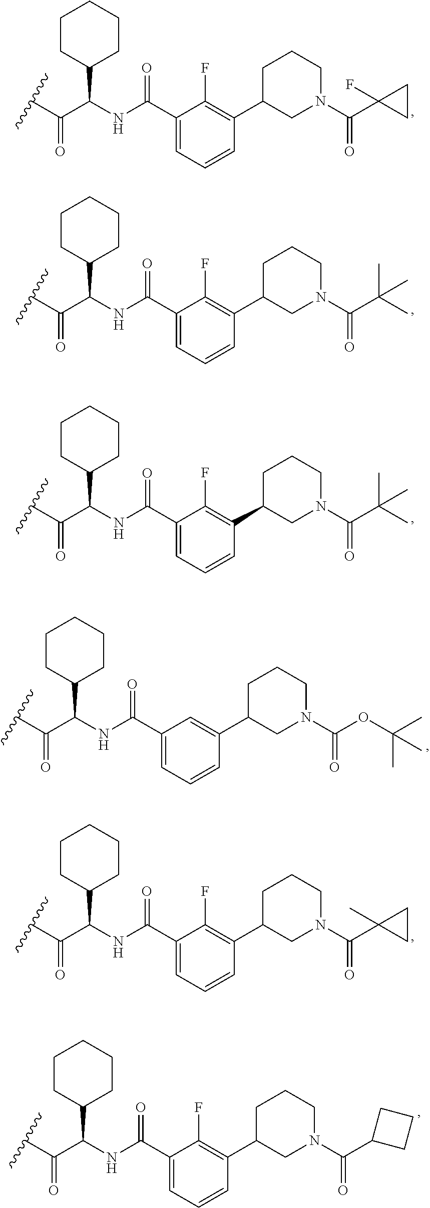 [SMILES: *C(=O)[C@H](NC(=O)c1cccc(C2CCCN(C(=O)C3(F)CC3)C2)c1F)C1CCCCC1 ; *C(=O)[C@H](NC(=O)c1cccc(C2CCCN(C(=O)C(C)(C)C)C2)c1F)C1CCCCC1 ; *C(=O)[C@H](NC(=O)c1cccc([C@H]2CCCN(C(=O)C(C)(C)C)C2)c1F)C1CCCCC1 ; *C(=O)[C@H](NC(=O)c1cccc(C2CCCN(C(=O)OC(C)(C)C)C2)c1)C1CCCCC1 ; *C(=O)[C@H](NC(=O)c1cccc(C2CCCN(C(=O)C3(C)CC3)C2)c1F)C1CCCCC1 ; *C(=O)[C@H](NC(=O)c1cccc(C2CCCN(C(=O)C3CCC3)C2)c1F)C1CCCCC1]

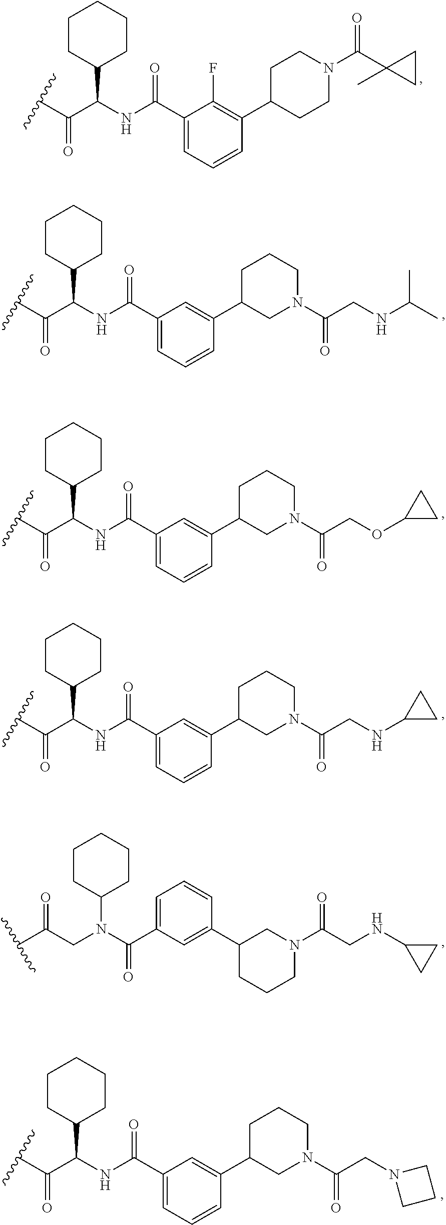 [SMILES: *C(=O)[C@H](NC(=O)c1cccc(C2CCN(C(=O)C3(C)CC3)CC2)c1F)C1CCCCC1 ; *C(=O)[C@H](NC(=O)c1cccc(C2CCCN(C(=O)CNC(C)C)C2)c1)C1CCCCC1 ; *C(=O)[C@H](NC(=O)c1cccc(C2CCCN(C(=O)COC3CC3)C2)c1)C1CCCCC1 ; *C(=O)[C@H](NC(=O)c1cccc(C2CCCN(C(=O)CNC3CC3)C2)c1)C1CCCCC1 ; *C(=O)CN(C(=O)c1cccc(C2CCCN(C(=O)CNC3CC3)C2)c1)C1CCCCC1 ; *C(=O)[C@H](NC(=O)c1cccc(C2CCCN(C(=O)CN3CCC3)C2)c1)C1CCCCC1]

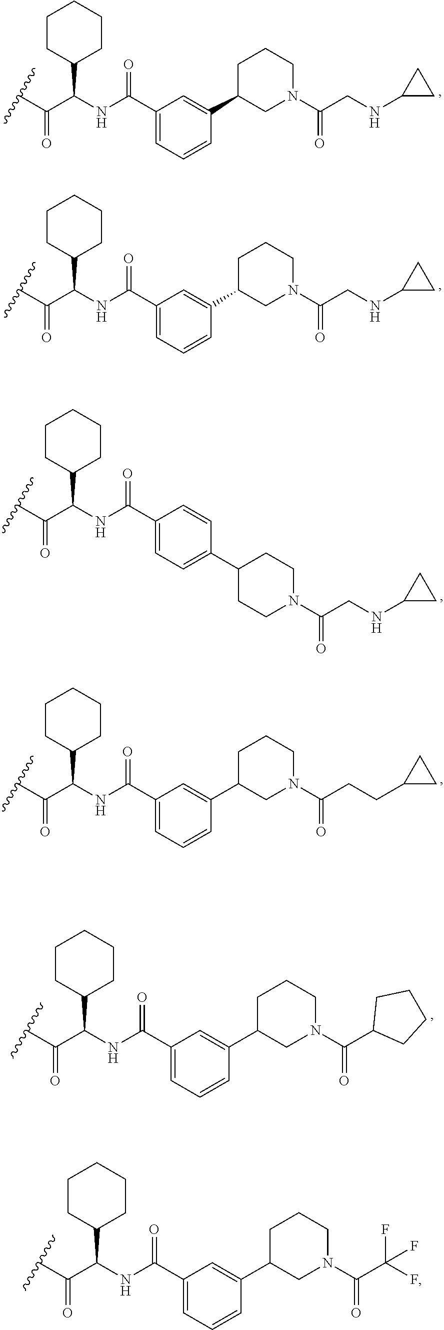 [SMILES: *C(=O)[C@H](NC(=O)c1cccc([C@H]2CCCN(C(=O)CNC3CC3)C2)c1)C1CCCCC1 ; *C(=O)[C@H](NC(=O)c1cccc([C@@H]2CCCN(C(=O)CNC3CC3)C2)c1)C1CCCCC1 ; *C(=O)[C@H](NC(=O)c1ccc(C2CCN(C(=O)CNC3CC3)CC2)cc1)C1CCCCC1 ; *C(=O)[C@H](NC(=O)c1cccc(C2CCCN(C(=O)CCC3CC3)C2)c1)C1CCCCC1 ; *C(=O)[C@H](NC(=O)c1cccc(C2CCCN(C(=O)C3CCCC3)C2)c1)C1CCCCC1 ; *C(=O)[C@H](NC(=O)c1cccc(C2CCCN(C(=O)C(F)(F)F)C2)c1)C1CCCCC1]

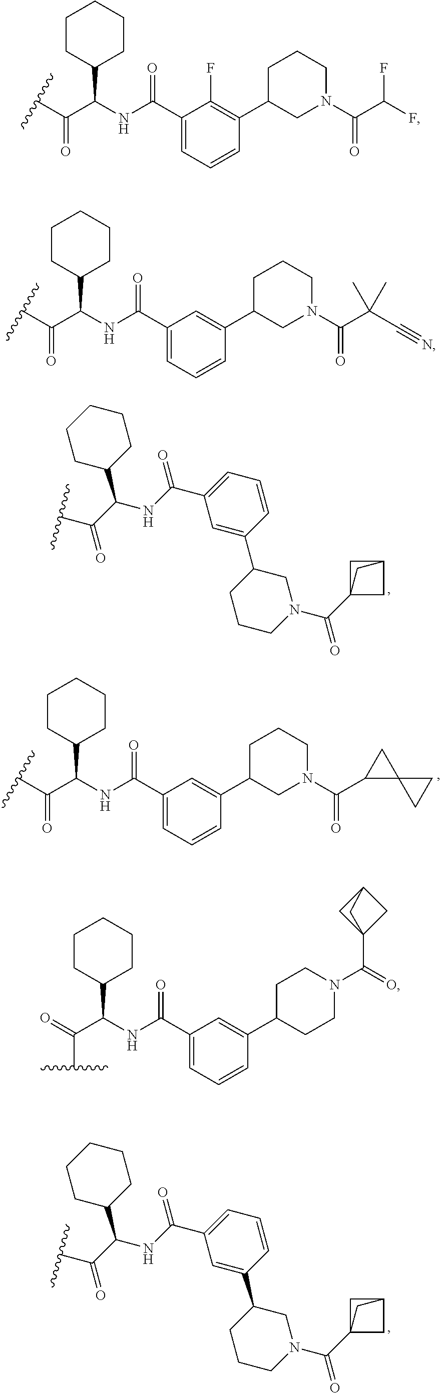 [SMILES: *C(=O)[C@H](NC(=O)c1cccc(C2CCCN(C(=O)C(F)F)C2)c1F)C1CCCCC1 ; *C(=O)[C@H](NC(=O)c1cccc(C2CCCN(C(=O)C(C)(C)C#N)C2)c1)C1CCCCC1 ; *C(=O)[C@H](NC(=O)c1cccc(C2CCCN(C(=O)C34CC(C3)C4)C2)c1)C1CCCCC1 ; *C(=O)[C@H](NC(=O)c1cccc(C2CCCN(C(=O)C3CC34CC4)C2)c1)C1CCCCC1 ; *C(=O)[C@H](NC(=O)c1cccc(C2CCN(C(=O)C34CC(C3)C4)CC2)c1)C1CCCCC1 ; *C(=O)[C@H](NC(=O)c1cccc([C@@H]2CCCN(C(=O)C34CC(C3)C4)C2)c1)C1CCCCC1]

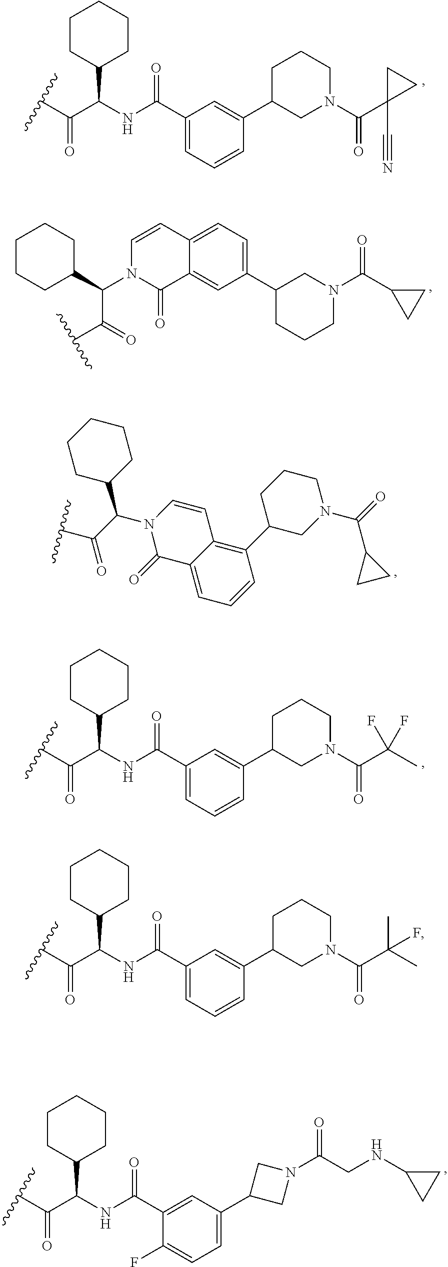 [SMILES: *C(=O)[C@H](NC(=O)c1cccc(C2CCCN(C(=O)C3(C#N)CC3)C2)c1)C1CCCCC1 ; *C(=O)[C@@H](C1CCCCC1)n1ccc2ccc(C3CCCN(C(=O)C4CC4)C3)cc2c1=O ; *C(=O)[C@@H](C1CCCCC1)n1ccc2c(C3CCCN(C(=O)C4CC4)C3)cccc2c1=O ; *C(=O)[C@H](NC(=O)c1cccc(C2CCCN(C(=O)C(C)(F)F)C2)c1)C1CCCCC1 ; *C(=O)[C@H](NC(=O)c1cccc(C2CCCN(C(=O)C(C)(C)F)C2)c1)C1CCCCC1 ; *C(=O)[C@H](NC(=O)c1cc(C2CN(C(=O)CNC3CC3)C2)ccc1F)C1CCCCC1]

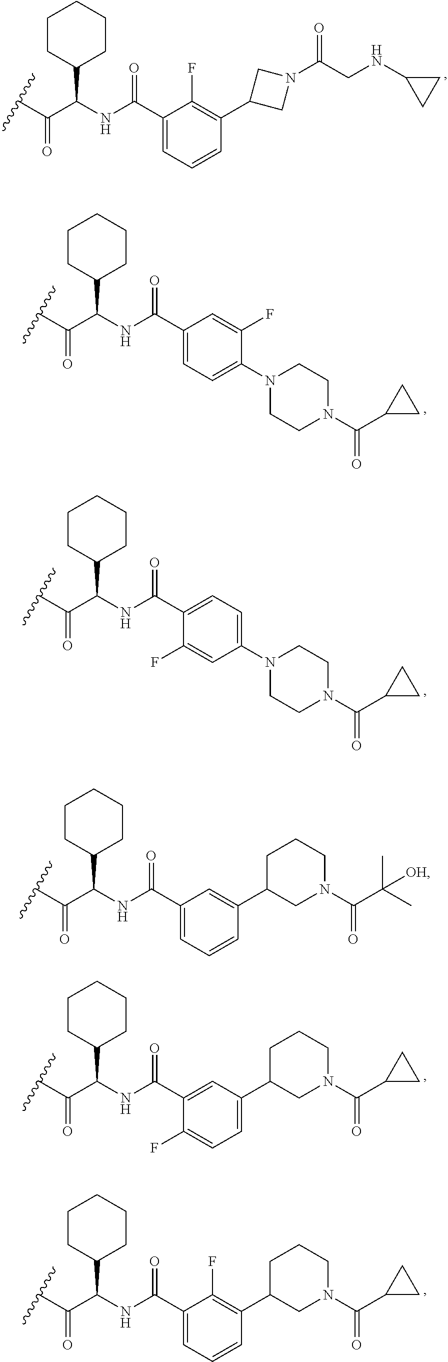 [SMILES: *C(=O)[C@H](NC(=O)c1cccc(C2CN(C(=O)CNC3CC3)C2)c1F)C1CCCCC1 ; *C(=O)[C@H](NC(=O)c1ccc(N2CCN(C(=O)C3CC3)CC2)c(F)c1)C1CCCCC1 ; *C(=O)[C@H](NC(=O)c1ccc(N2CCN(C(=O)C3CC3)CC2)cc1F)C1CCCCC1 ; *C(=O)[C@H](NC(=O)c1cccc(C2CCCN(C(=O)C(C)(C)O)C2)c1)C1CCCCC1 ; *C(=O)[C@H](NC(=O)c1cc(C2CCCN(C(=O)C3CC3)C2)ccc1F)C1CCCCC1 ; *C(=O)[C@H](NC(=O)c1cccc(C2CCCN(C(=O)C3CC3)C2)c1F)C1CCCCC1]

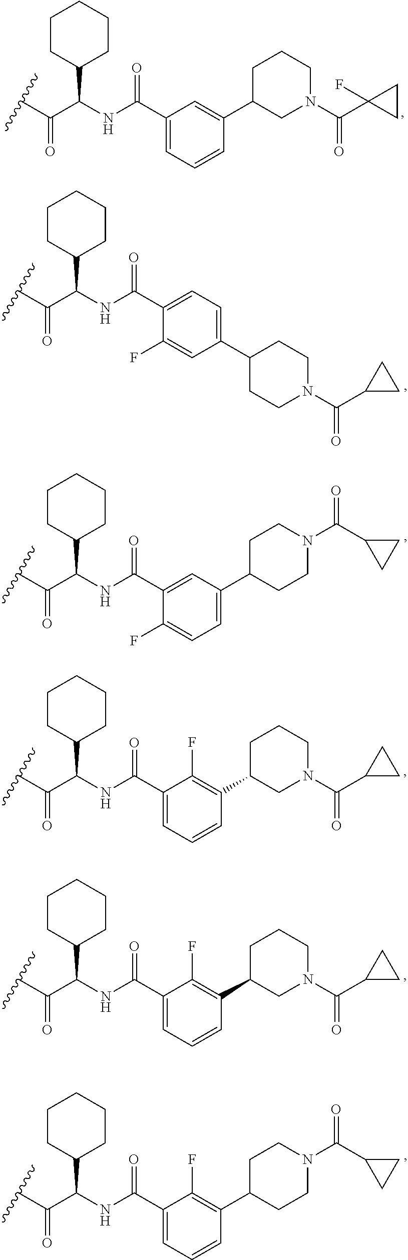 [SMILES: *C(=O)[C@H](NC(=O)c1cccc(C2CCCN(C(=O)C3(F)CC3)C2)c1)C1CCCCC1 ; *C(=O)[C@H](NC(=O)c1ccc(C2CCN(C(=O)C3CC3)CC2)cc1F)C1CCCCC1 ; *C(=O)[C@H](NC(=O)c1cc(C2CCN(C(=O)C3CC3)CC2)ccc1F)C1CCCCC1 ; *C(=O)[C@H](NC(=O)c1cccc([C@@H]2CCCN(C(=O)C3CC3)C2)c1F)C1CCCCC1 ; *C(=O)[C@H](NC(=O)c1cccc([C@H]2CCCN(C(=O)C3CC3)C2)c1F)C1CCCCC1 ; *C(=O)[C@H](NC(=O)c1cccc(C2CCN(C(=O)C3CC3)CC2)c1F)C1CCCCC1]

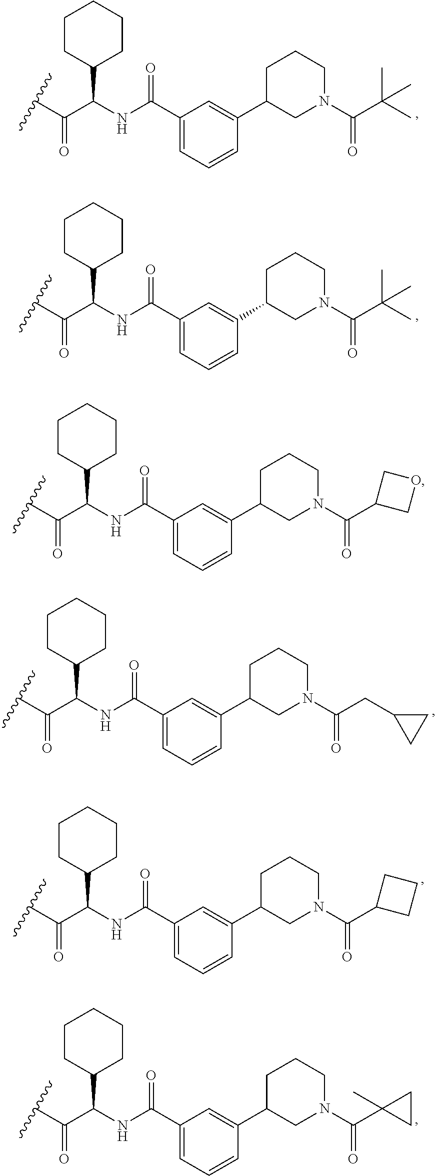 [SMILES: *C(=O)[C@H](NC(=O)c1cccc(C2CCCN(C(=O)C(C)(C)C)C2)c1)C1CCCCC1 ; *C(=O)[C@H](NC(=O)c1cccc([C@@H]2CCCN(C(=O)C(C)(C)C)C2)c1)C1CCCCC1 ; *C(=O)[C@H](NC(=O)c1cccc(C2CCCN(C(=O)C3COC3)C2)c1)C1CCCCC1 ; *C(=O)[C@H](NC(=O)c1cccc(C2CCCN(C(=O)CC3CC3)C2)c1)C1CCCCC1 ; *C(=O)[C@H](NC(=O)c1cccc(C2CCCN(C(=O)C3CCC3)C2)c1)C1CCCCC1 ; *C(=O)[C@H](NC(=O)c1cccc(C2CCCN(C(=O)C3(C)CC3)C2)c1)C1CCCCC1]

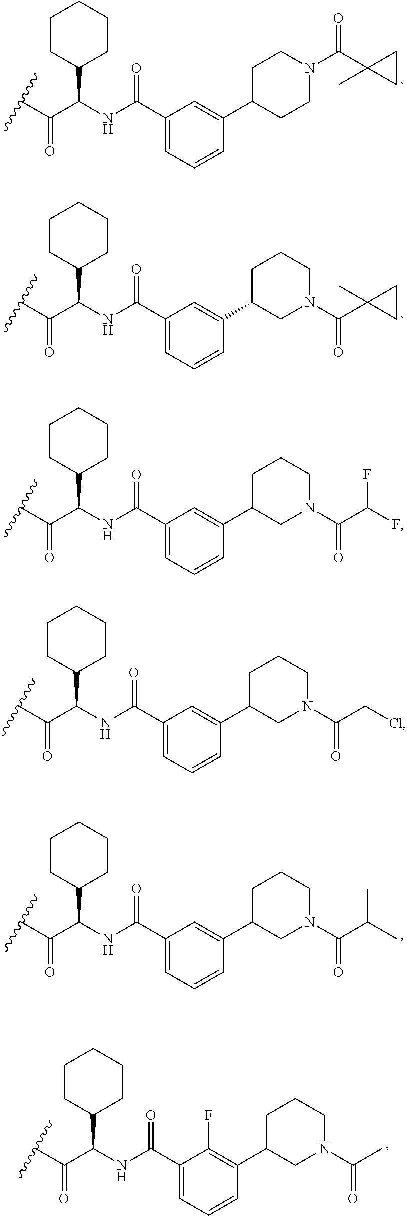 [SMILES: *C(=O)[C@H](NC(=O)c1cccc(C2CCN(C(=O)C3(C)CC3)CC2)c1)C1CCCCC1 ; *C(=O)[C@H](NC(=O)c1cccc([C@@H]2CCCN(C(=O)C3(C)CC3)C2)c1)C1CCCCC1 ; *C(=O)[C@H](NC(=O)c1cccc(C2CCCN(C(=O)C(F)F)C2)c1)C1CCCCC1 ; *C(=O)[C@H](NC(=O)c1cccc(C2CCCN(C(=O)CCl)C2)c1)C1CCCCC1 ; *C(=O)[C@H](NC(=O)c1cccc(C2CCCN(C(=O)C(C)C)C2)c1)C1CCCCC1 ; *C(=O)[C@H](NC(=O)c1cccc(C2CCCN(C(C)=O)C2)c1F)C1CCCCC1]

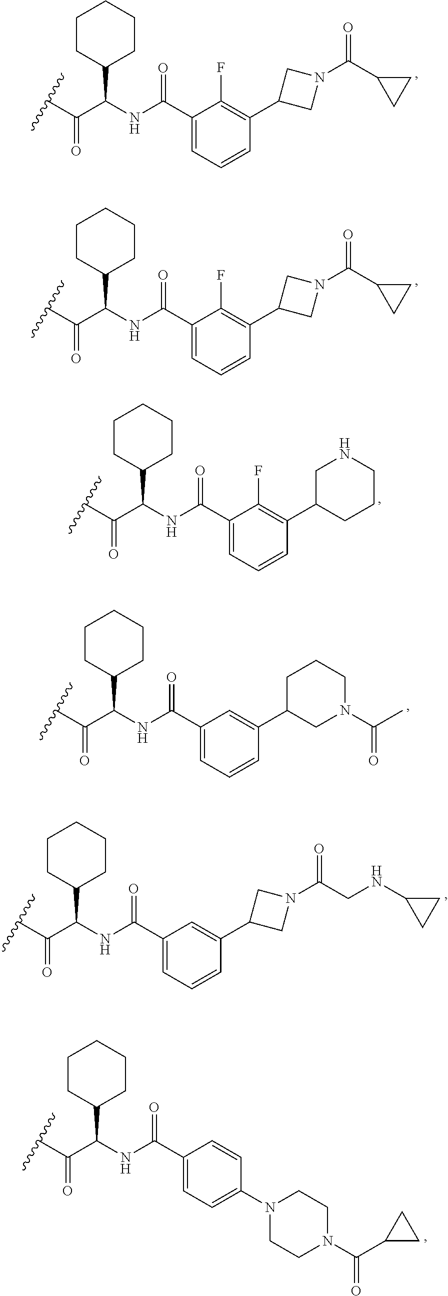 [SMILES: *C(=O)[C@H](NC(=O)c1cccc(C2CN(C(=O)C3CC3)C2)c1F)C1CCCCC1 ; *C(=O)[C@H](NC(=O)c1cccc(C2CN(C(=O)C3CC3)C2)c1F)C1CCCCC1 ; *C(=O)[C@H](NC(=O)c1cccc(C2CCCNC2)c1F)C1CCCCC1 ; *C(=O)[C@H](NC(=O)c1cccc(C2CCCN(C(C)=O)C2)c1)C1CCCCC1 ; *C(=O)[C@H](NC(=O)c1cccc(C2CN(C(=O)CNC3CC3)C2)c1)C1CCCCC1 ; *C(=O)[C@H](NC(=O)c1ccc(N2CCN(C(=O)C3CC3)CC2)cc1)C1CCCCC1]

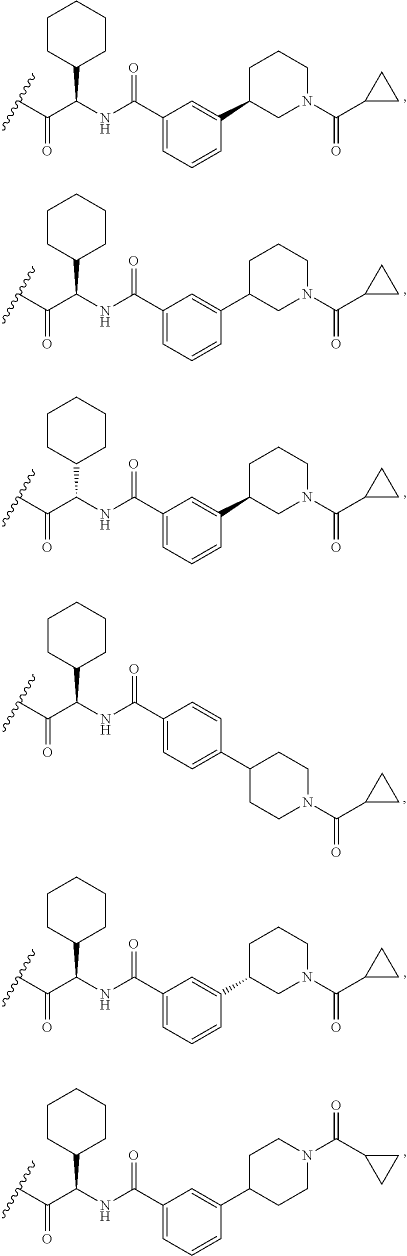 [SMILES: *C(=O)[C@H](NC(=O)c1cccc([C@H]2CCCN(C(=O)C3CC3)C2)c1)C1CCCCC1 ; *C(=O)[C@H](NC(=O)c1cccc(C2CCCN(C(=O)C3CC3)C2)c1)C1CCCCC1 ; *C(=O)[C@@H](NC(=O)c1cccc([C@H]2CCCN(C(=O)C3CC3)C2)c1)C1CCCCC1 ; *C(=O)[C@H](NC(=O)c1ccc(C2CCN(C(=O)C3CC3)CC2)cc1)C1CCCCC1 ; *C(=O)[C@H](NC(=O)c1cccc([C@@H]2CCCN(C(=O)C3CC3)C2)c1)C1CCCCC1 ; *C(=O)[C@H](NC(=O)c1cccc(C2CCN(C(=O)C3CC3)CC2)c1)C1CCCCC1]

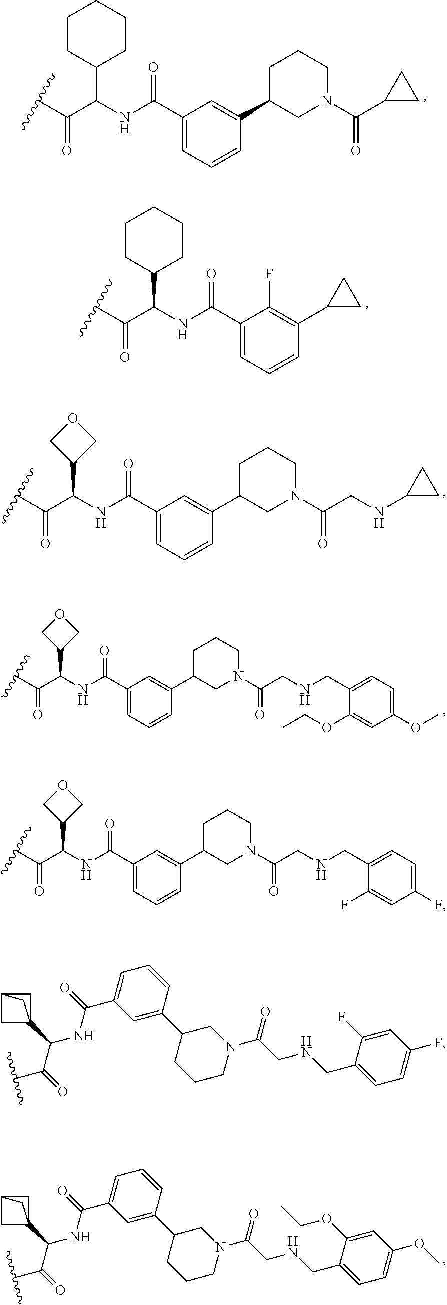

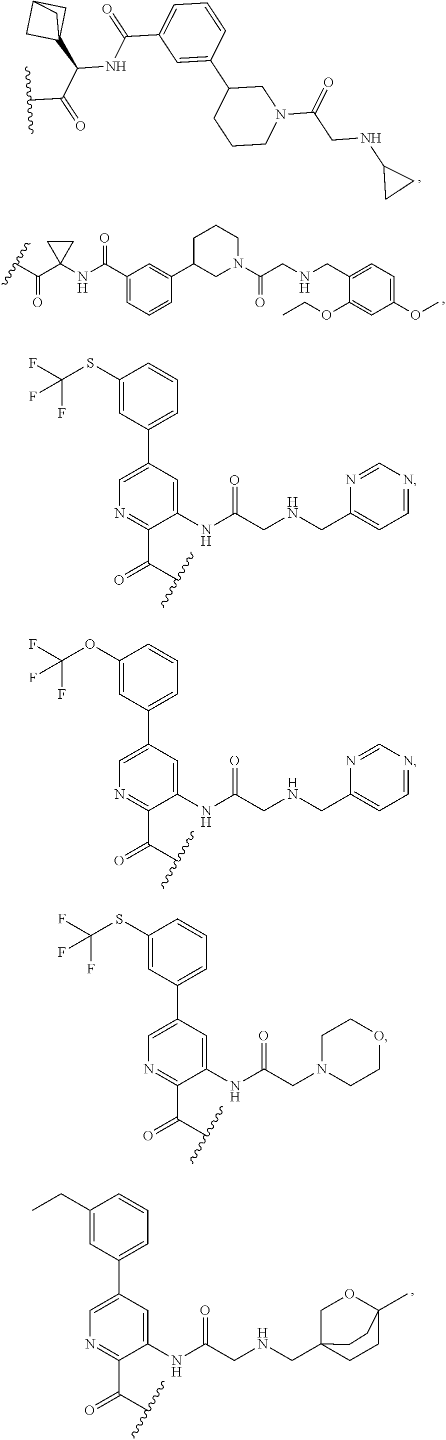

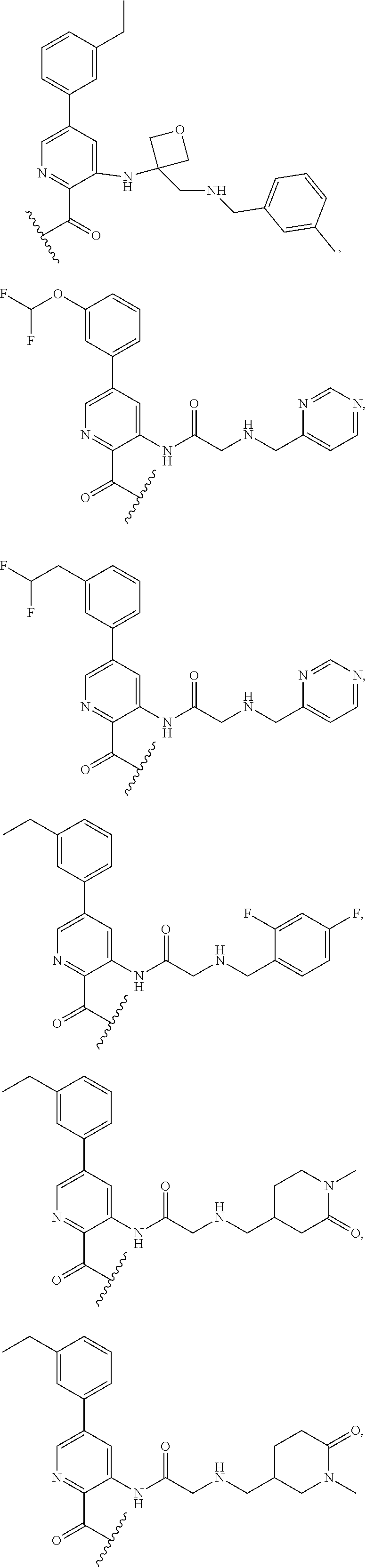

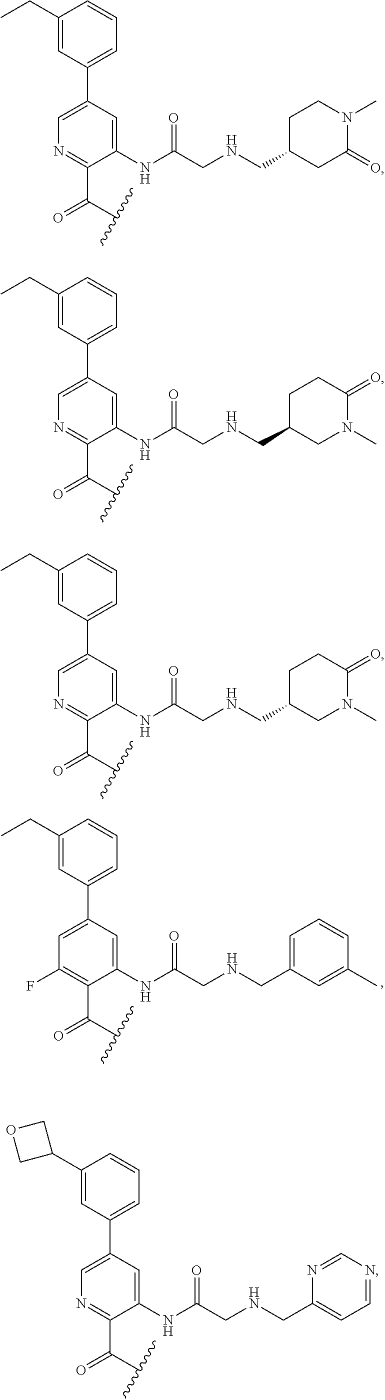

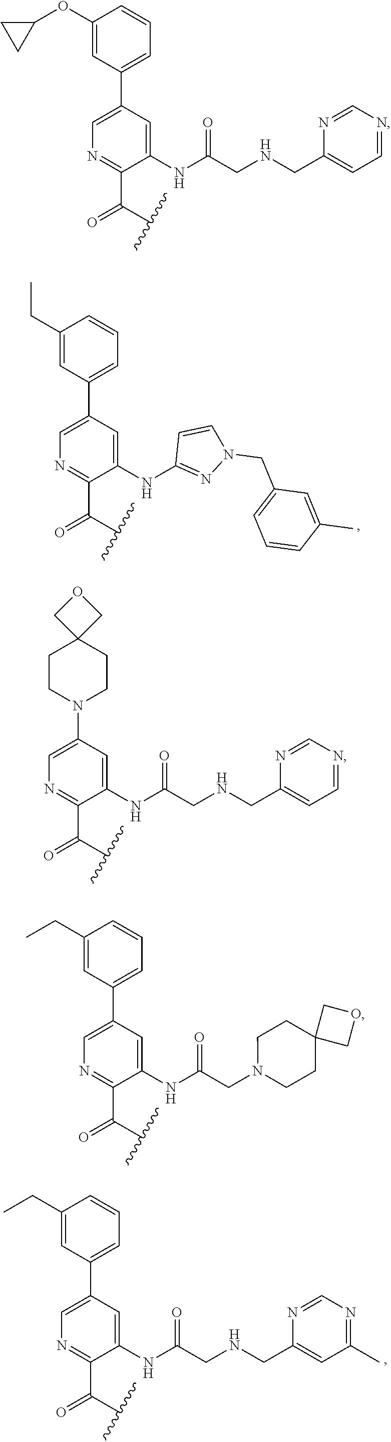

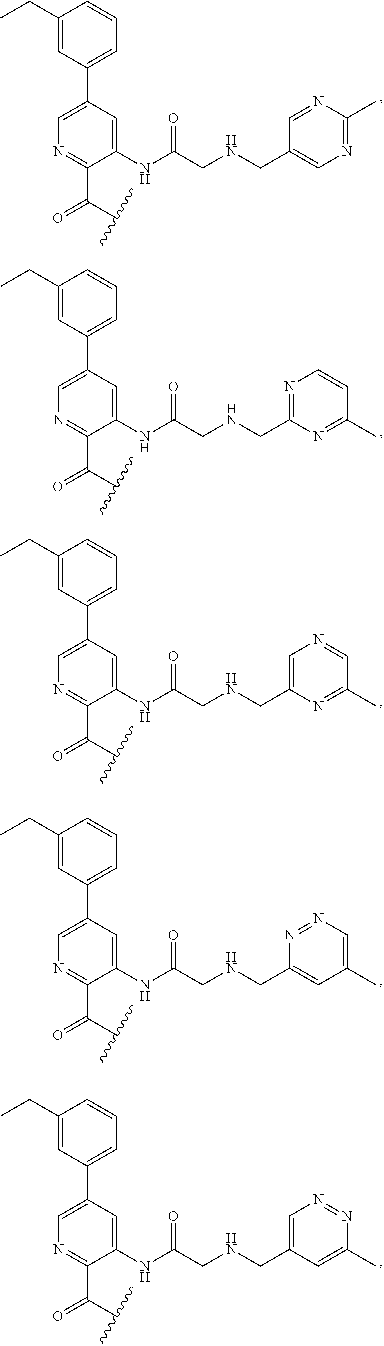

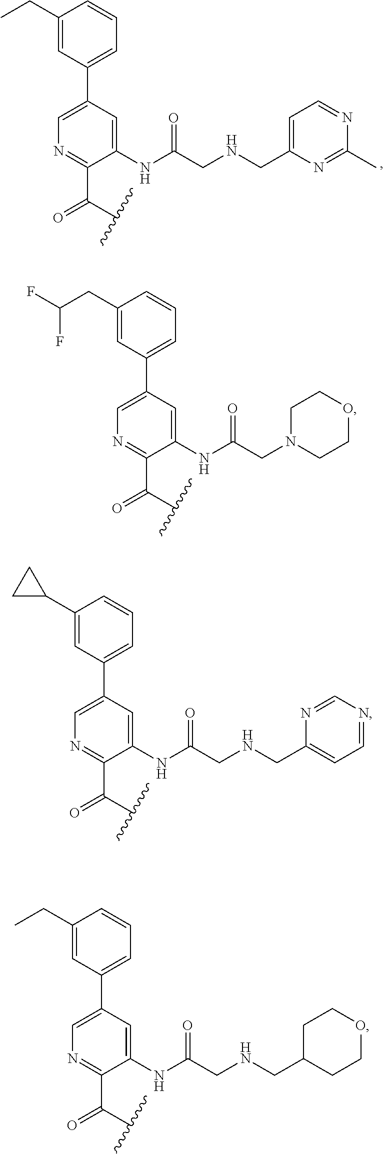

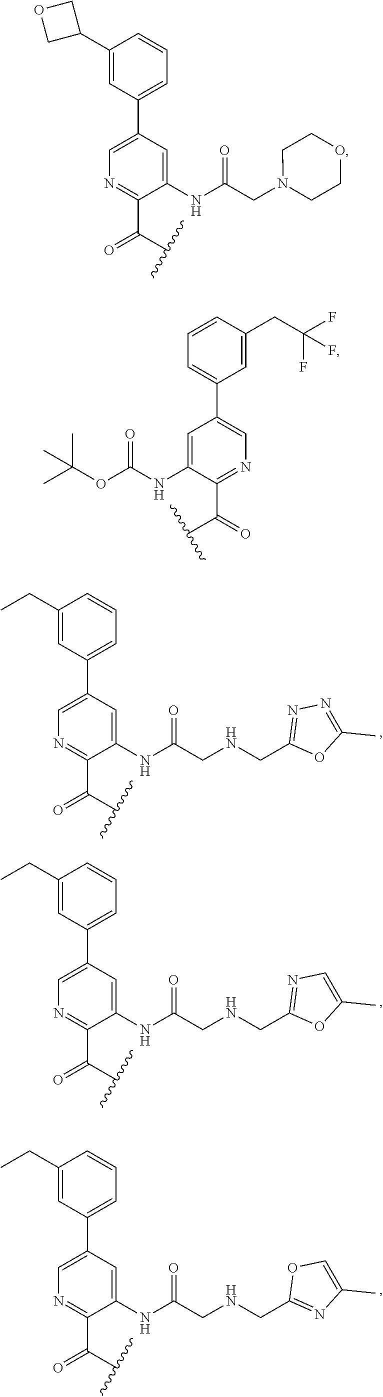

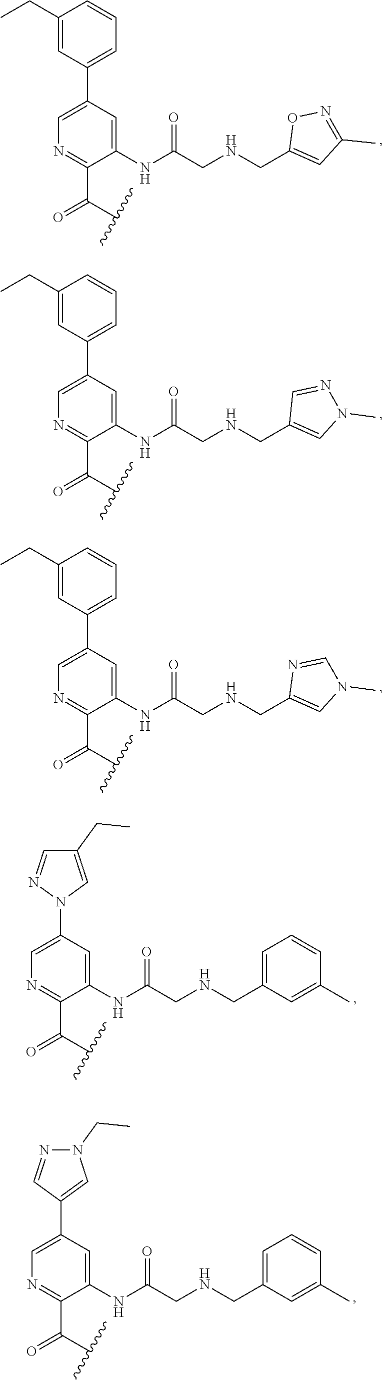

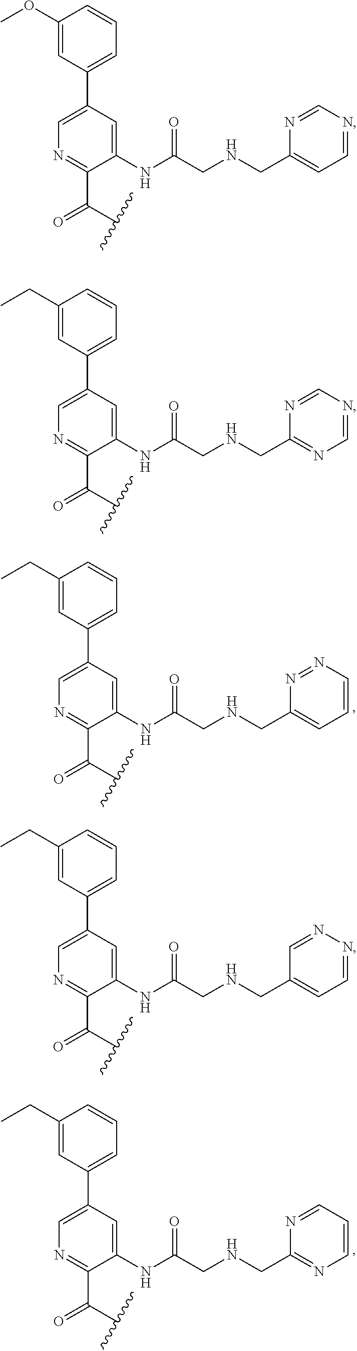 [SMILES: *C(=O)c1ncc(-c2cccc(OC)c2)cc1NC(=O)CNCc1ccncn1 ; *C(=O)c1ncc(-c2cccc(CC)c2)cc1NC(=O)CNCc1ncncn1 ; *C(=O)c1ncc(-c2cccc(CC)c2)cc1NC(=O)CNCc1cccnn1 ; *C(=O)c1ncc(-c2cccc(CC)c2)cc1NC(=O)CNCc1ccnnc1 ; *C(=O)c1ncc(-c2cccc(CC)c2)cc1NC(=O)CNCc1ncccn1]

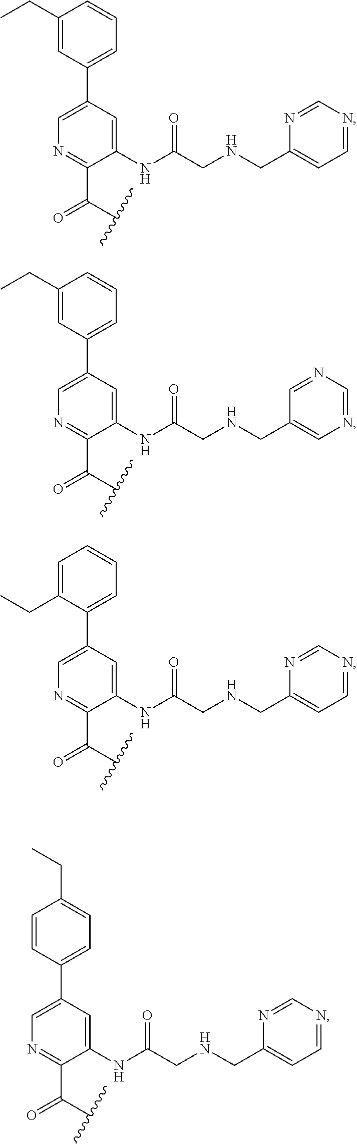 [SMILES: *C(=O)c1ncc(-c2cccc(CC)c2)cc1NC(=O)CNCc1ccncn1 ; *C(=O)c1ncc(-c2cccc(CC)c2)cc1NC(=O)CNCc1cncnc1 ; *C(=O)c1ncc(-c2ccccc2CC)cc1NC(=O)CNCc1ccncn1 ; *C(=O)c1ncc(-c2ccc(CC)cc2)cc1NC(=O)CNCc1ccncn1]

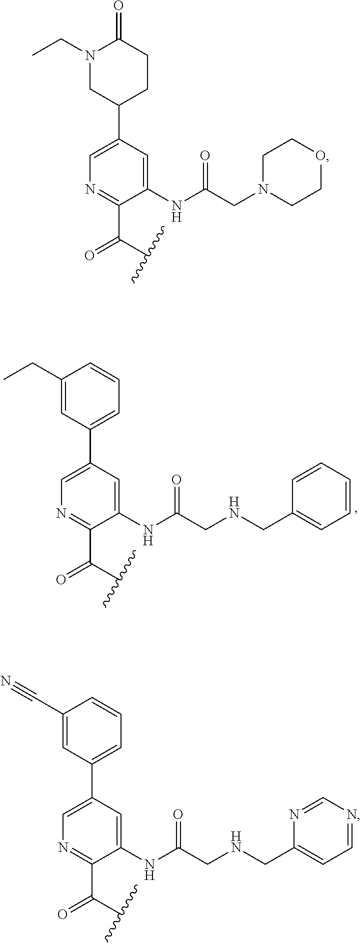 [SMILES: *C(=O)c1ncc(C2CCC(=O)N(CC)C2)cc1NC(=O)CN1CCOCC1 ; *C(=O)c1ncc(-c2cccc(CC)c2)cc1NC(=O)CNCc1ccccc1 ; *C(=O)c1ncc(-c2cccc(C#N)c2)cc1NC(=O)CNCc1ccncn1]

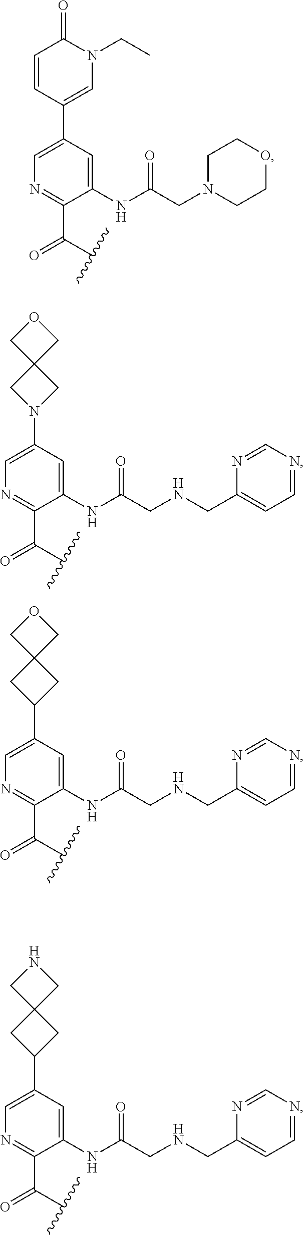 [SMILES: *C(=O)c1ncc(-c2ccc(=O)n(CC)c2)cc1NC(=O)CN1CCOCC1 ; *C(=O)c1ncc(N2CC3(COC3)C2)cc1NC(=O)CNCc1ccncn1 ; *C(=O)c1ncc(C2CC3(COC3)C2)cc1NC(=O)CNCc1ccncn1 ; *C(=O)c1ncc(C2CC3(CNC3)C2)cc1NC(=O)CNCc1ccncn1]

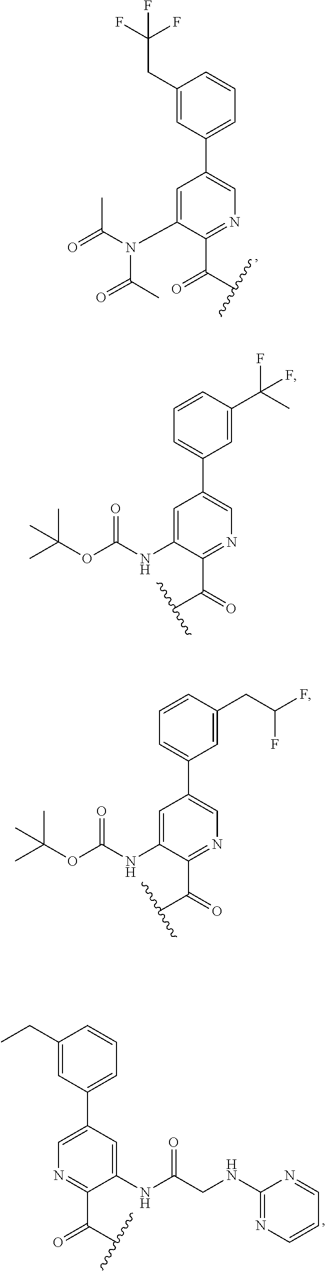 [SMILES: *C(=O)c1ncc(-c2cccc(CC(F)(F)F)c2)cc1N(C(C)=O)C(C)=O ; *C(=O)c1ncc(-c2cccc(C(C)(F)F)c2)cc1NC(=O)OC(C)(C)C ; *C(=O)c1ncc(-c2cccc(CC(F)F)c2)cc1NC(=O)OC(C)(C)C ; *C(=O)c1ncc(-c2cccc(CC)c2)cc1NC(=O)CNc1ncccn1]

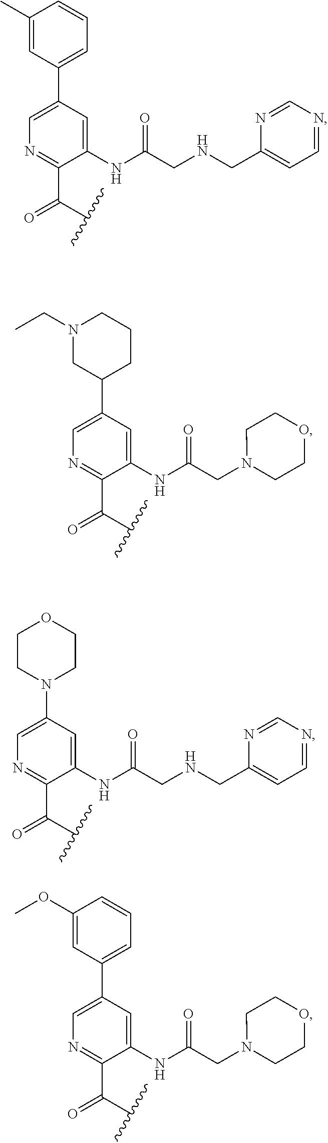 [SMILES: *C(=O)c1ncc(-c2cccc(C)c2)cc1NC(=O)CNCc1ccncn1 ; *C(=O)c1ncc(C2CCCN(CC)C2)cc1NC(=O)CN1CCOCC1 ; *C(=O)c1ncc(N2CCOCC2)cc1NC(=O)CNCc1ccncn1 ; *C(=O)c1ncc(-c2cccc(OC)c2)cc1NC(=O)CN1CCOCC1]

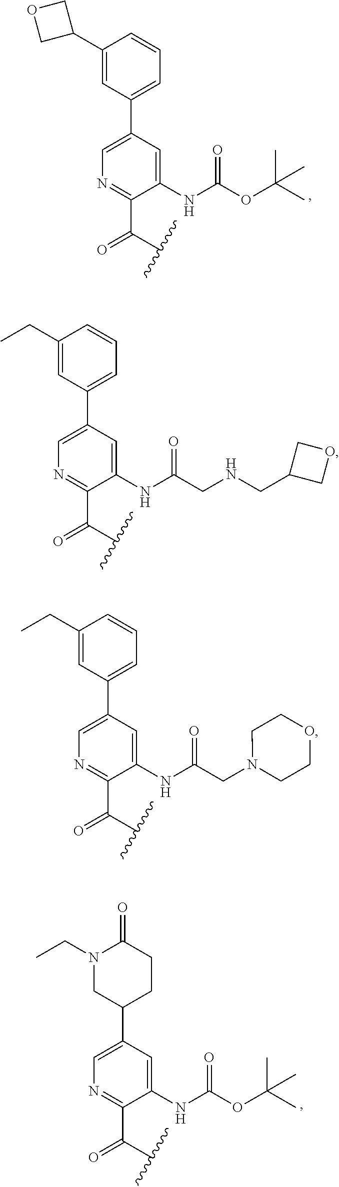

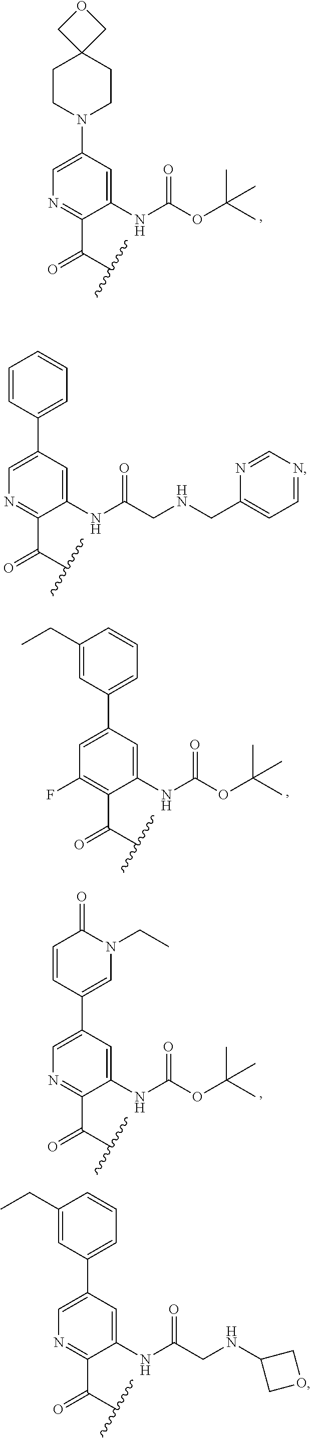

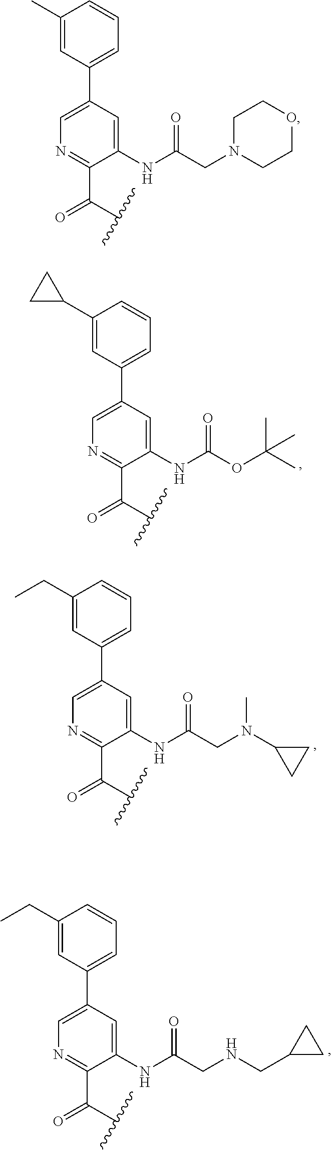

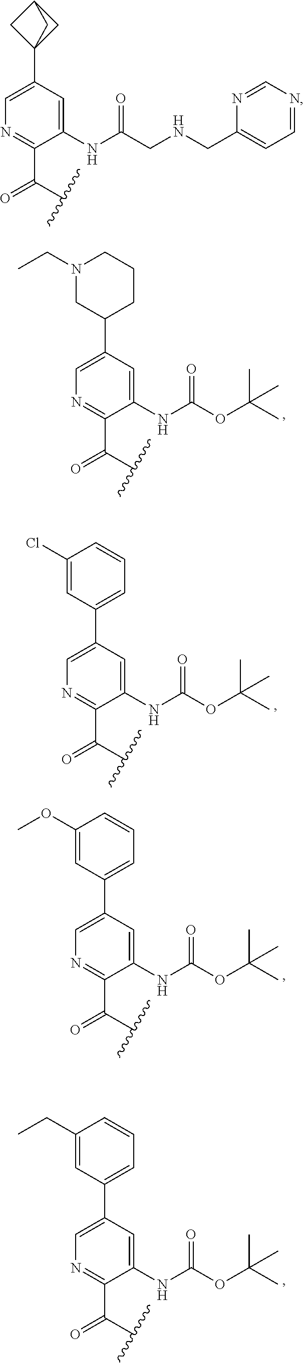

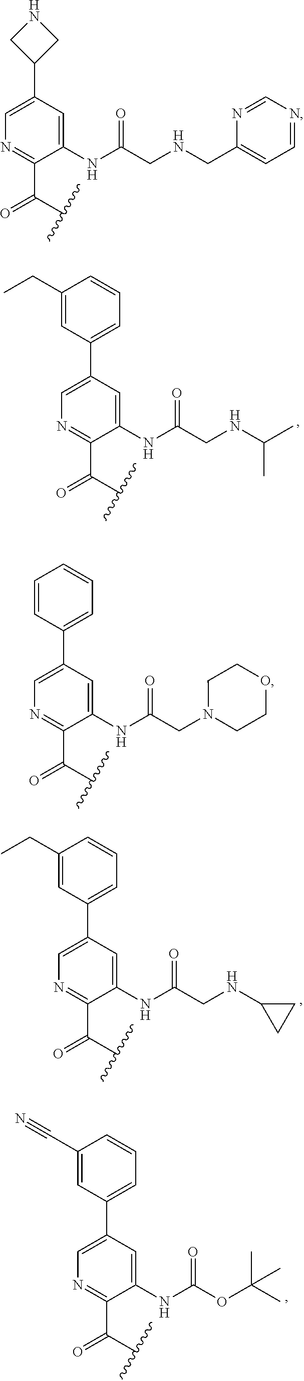

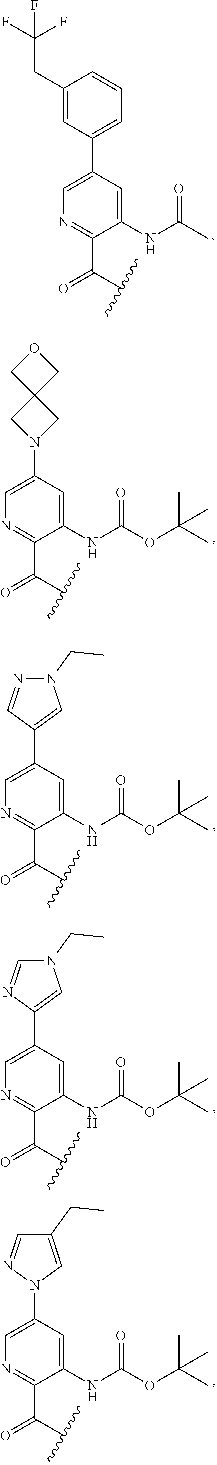

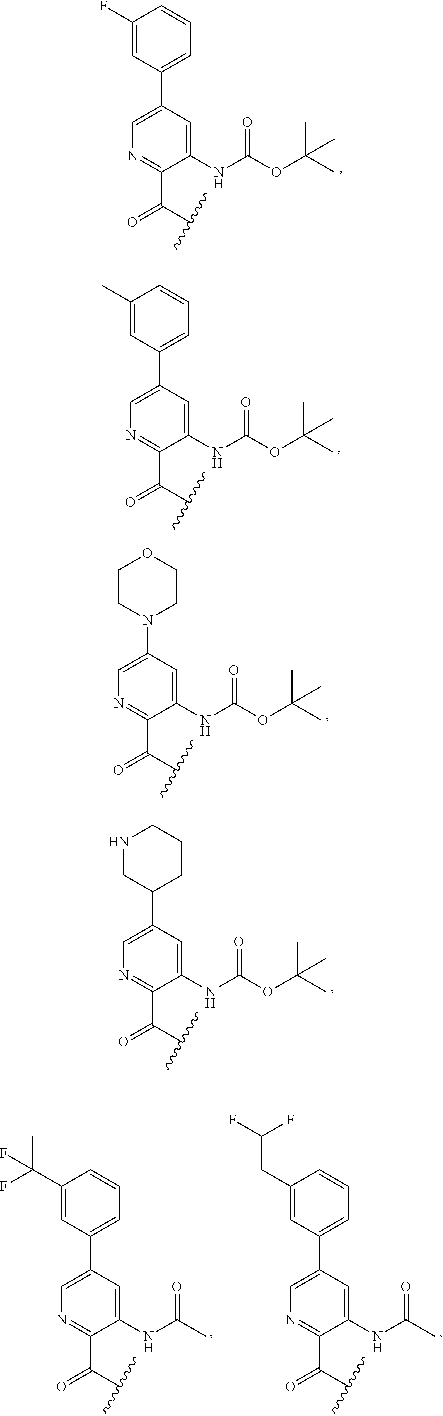 [SMILES: *C(=O)c1ncc(-c2cccc(F)c2)cc1NC(=O)OC(C)(C)C ; *C(=O)c1ncc(-c2cccc(C)c2)cc1NC(=O)OC(C)(C)C ; *C(=O)c1ncc(N2CCOCC2)cc1NC(=O)OC(C)(C)C ; *C(=O)c1ncc(C2CCCNC2)cc1NC(=O)OC(C)(C)C ; *C(=O)c1ncc(-c2cccc(C(C)(F)F)c2)cc1NC(C)=O ; *C(=O)c1ncc(-c2cccc(CC(F)F)c2)cc1NC(C)=O]

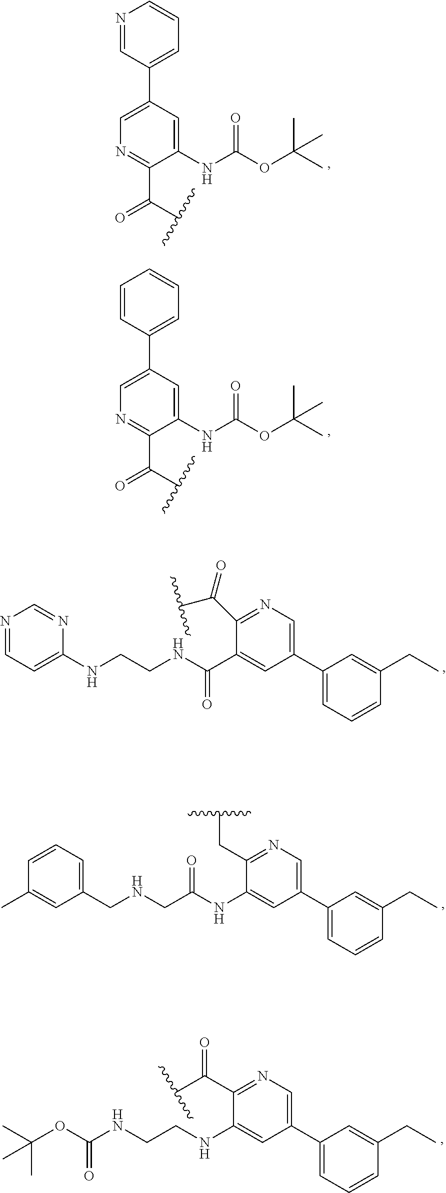

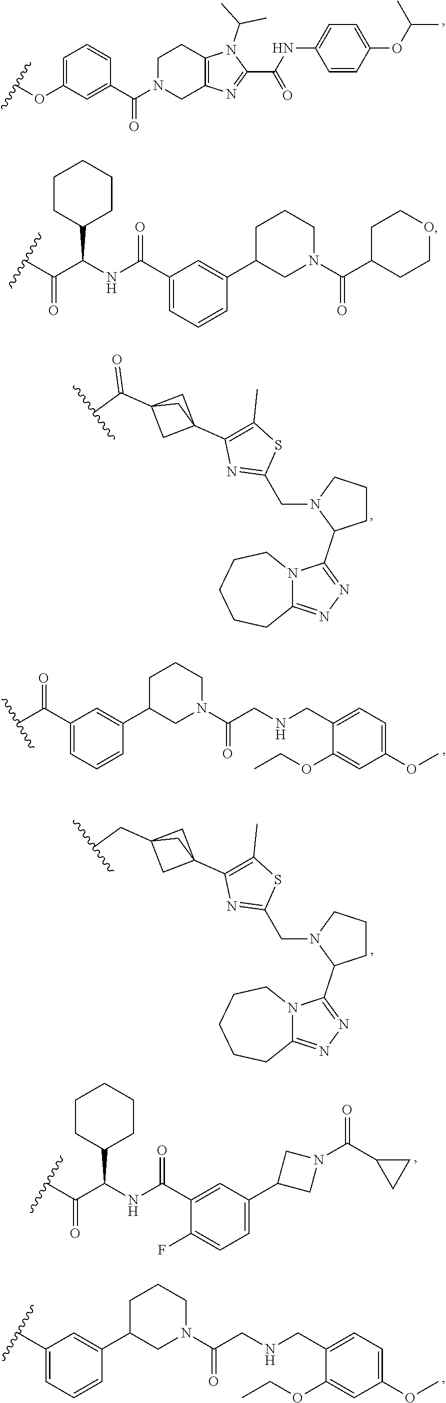

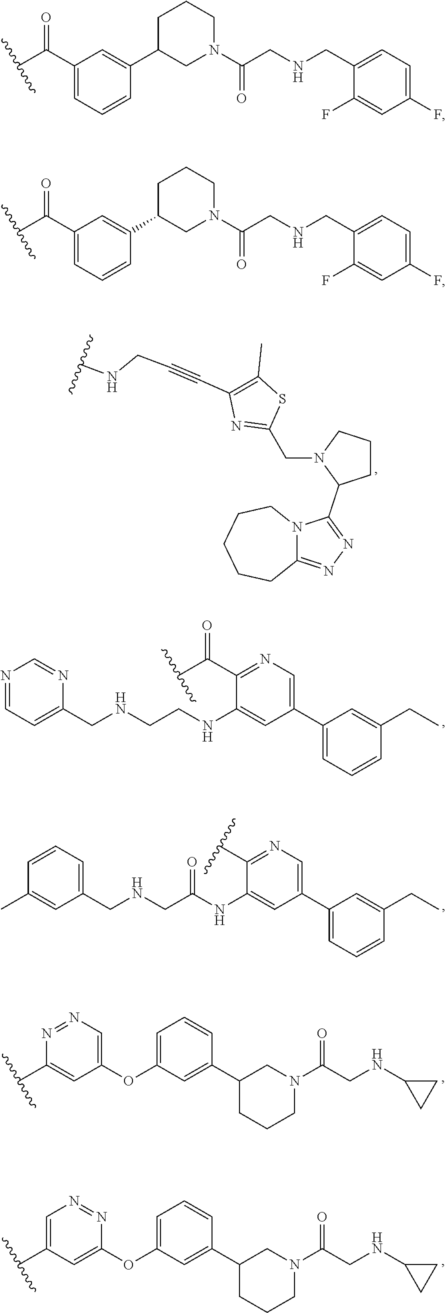

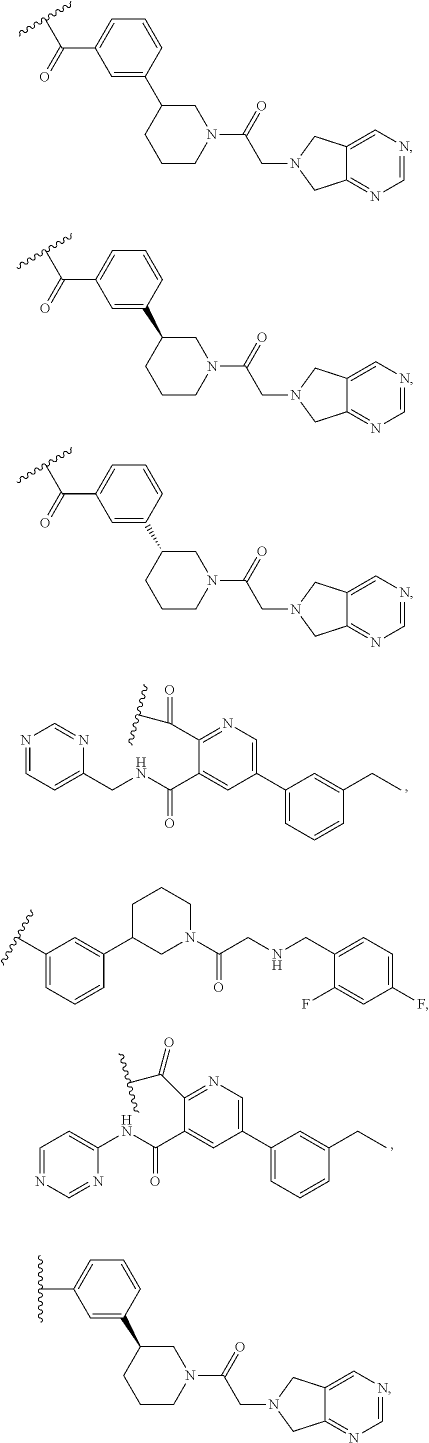

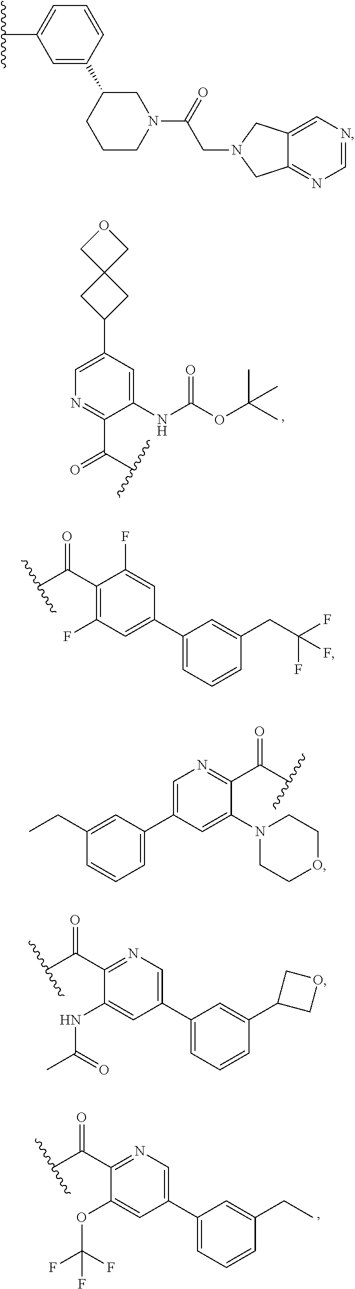 [SMILES: *c1cccc([C@H]2CCCN(C(=O)CN3Cc4cncnc4C3)C2)c1 ; *C(=O)c1ncc(C2CC3(COC3)C2)cc1NC(=O)OC(C)(C)C ; *C(=O)c1c(F)cc(-c2cccc(CC(F)(F)F)c2)cc1F ; *C(=O)c1ncc(-c2cccc(CC)c2)cc1N1CCOCC1 ; *C(=O)c1ncc(-c2cccc(C3COC3)c2)cc1NC(C)=O ; *C(=O)c1ncc(-c2cccc(CC)c2)cc1OC(F)(F)F]

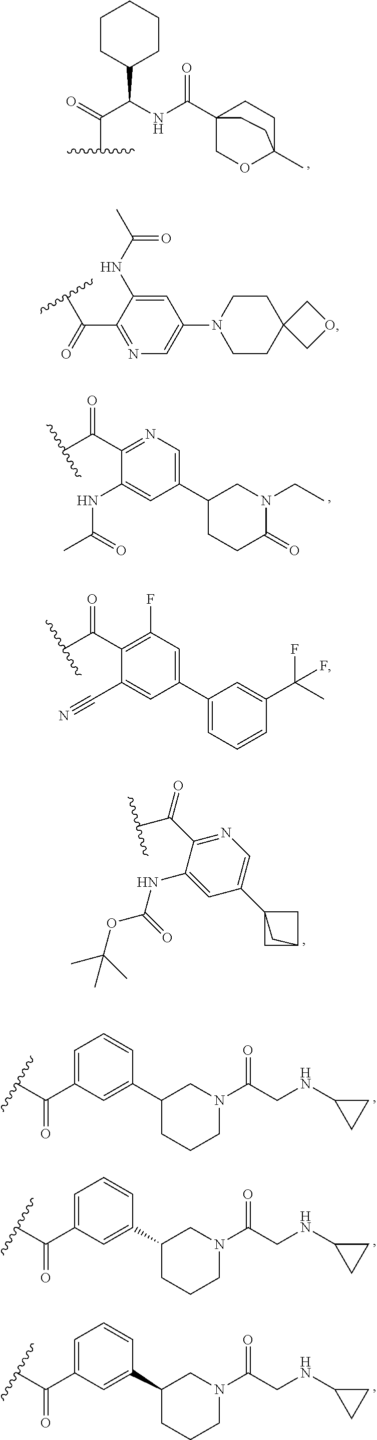

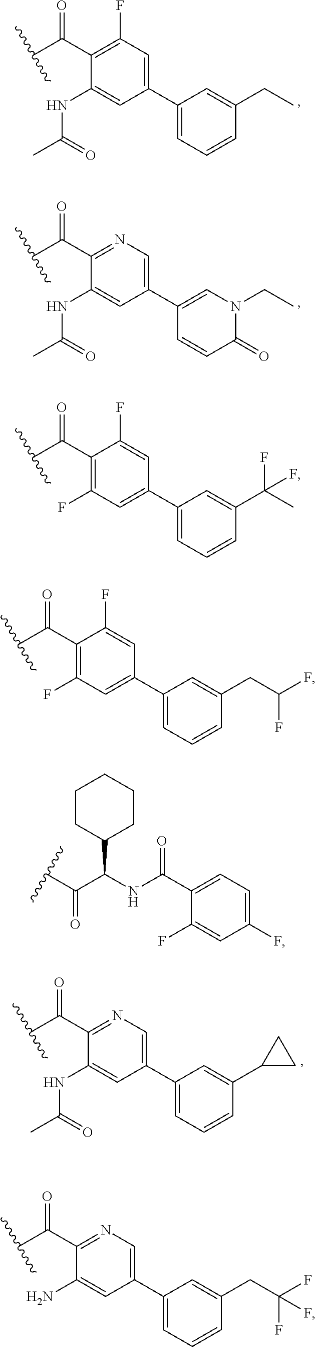

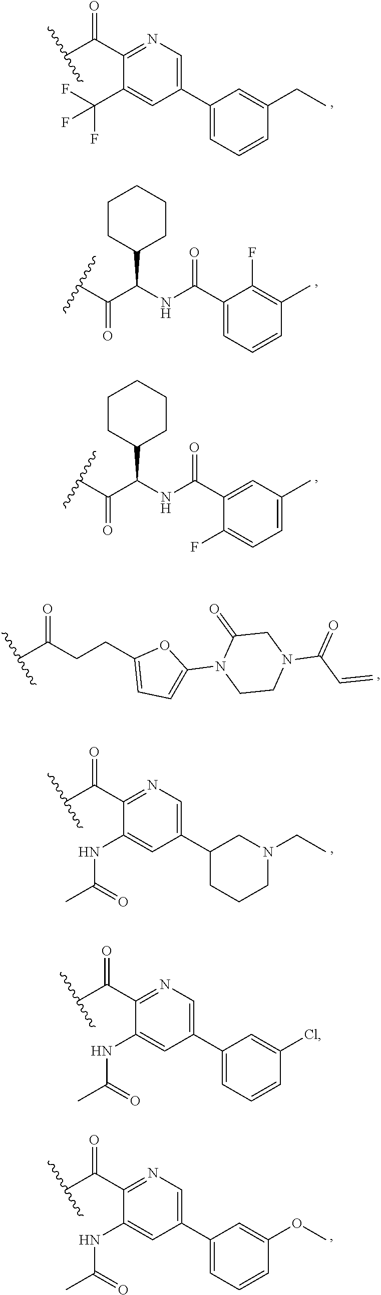

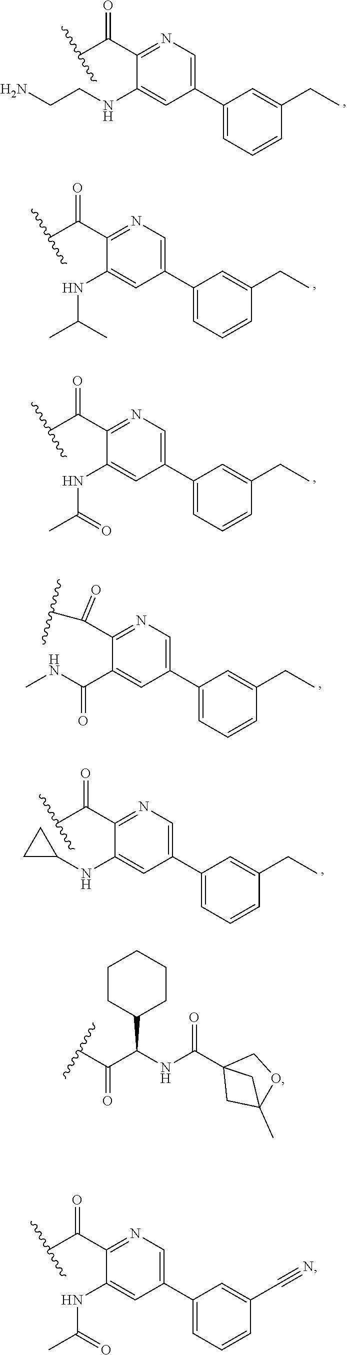

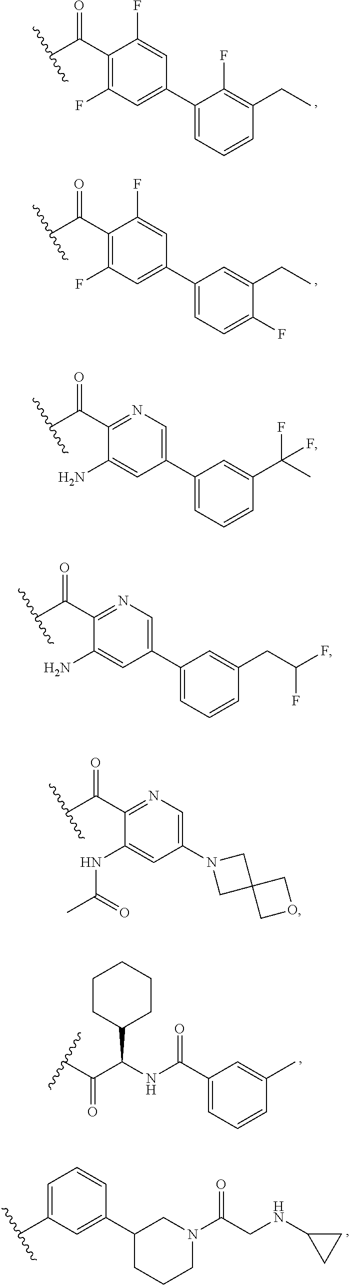

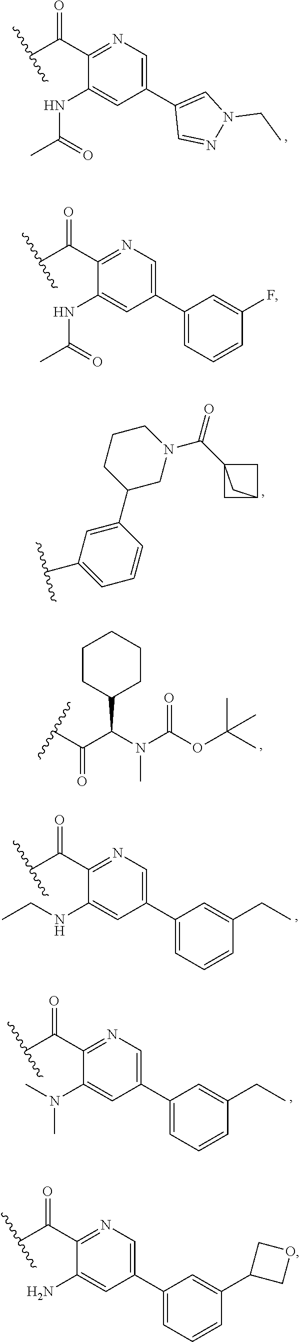

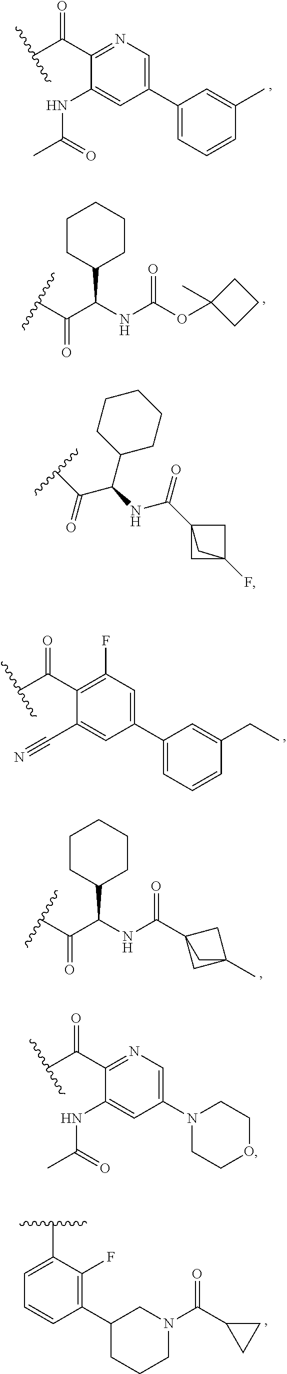

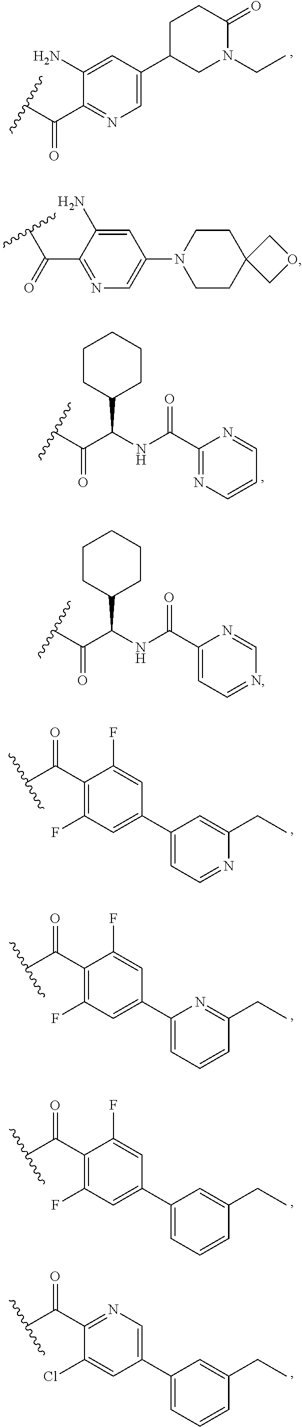

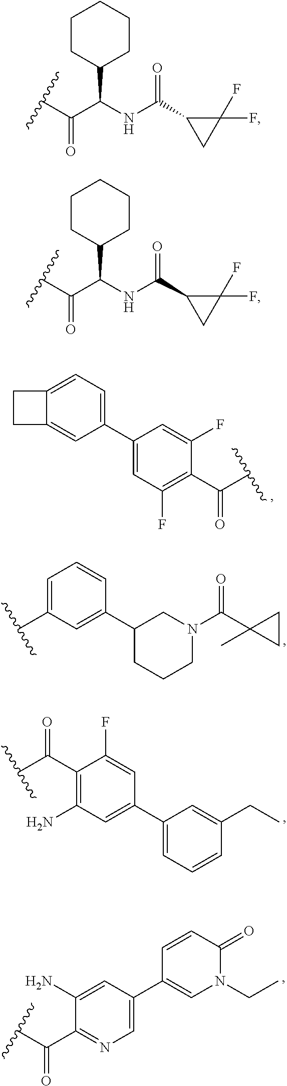

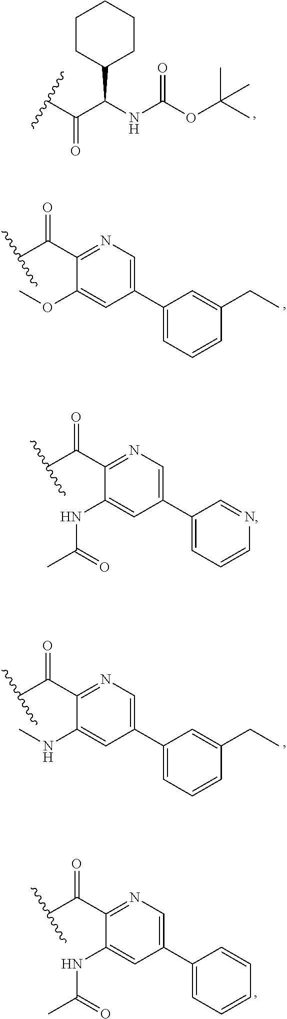 [SMILES: *C(=O)[C@H](NC(=O)OC(C)(C)C)C1CCCCC1 ; *C(=O)c1ncc(-c2cccc(CC)c2)cc1OC ; *C(=O)c1ncc(-c2cccnc2)cc1NC(C)=O ; *C(=O)c1ncc(-c2cccc(CC)c2)cc1NC ; *C(=O)c1ncc(-c2ccccc2)cc1NC(C)=O]

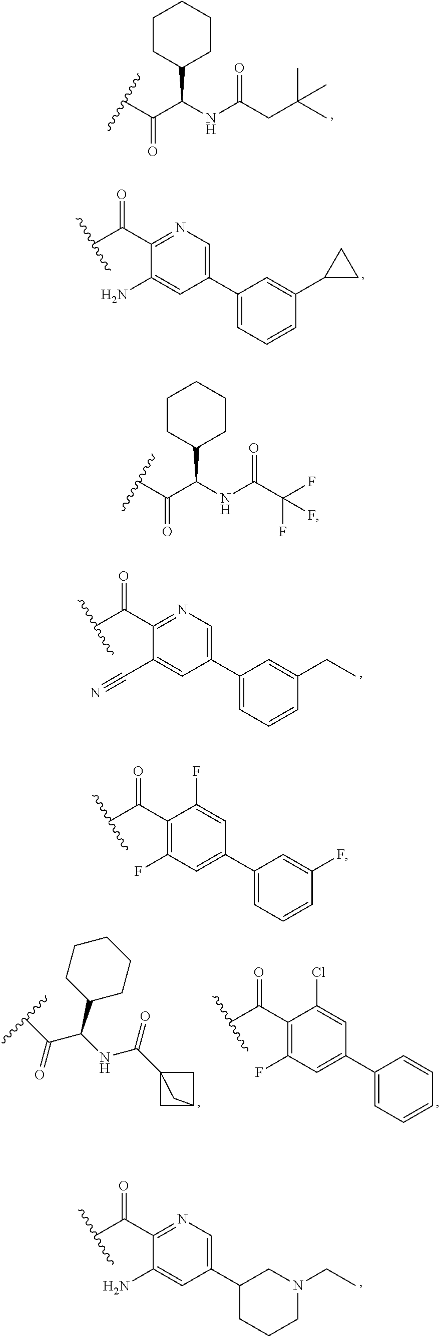

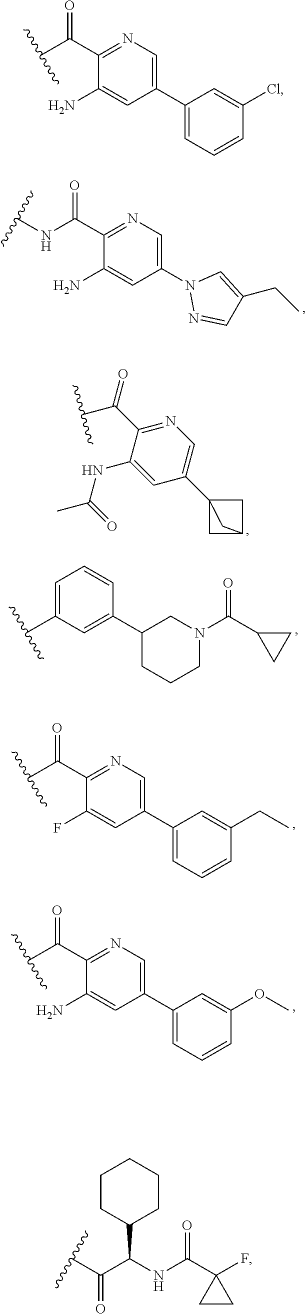

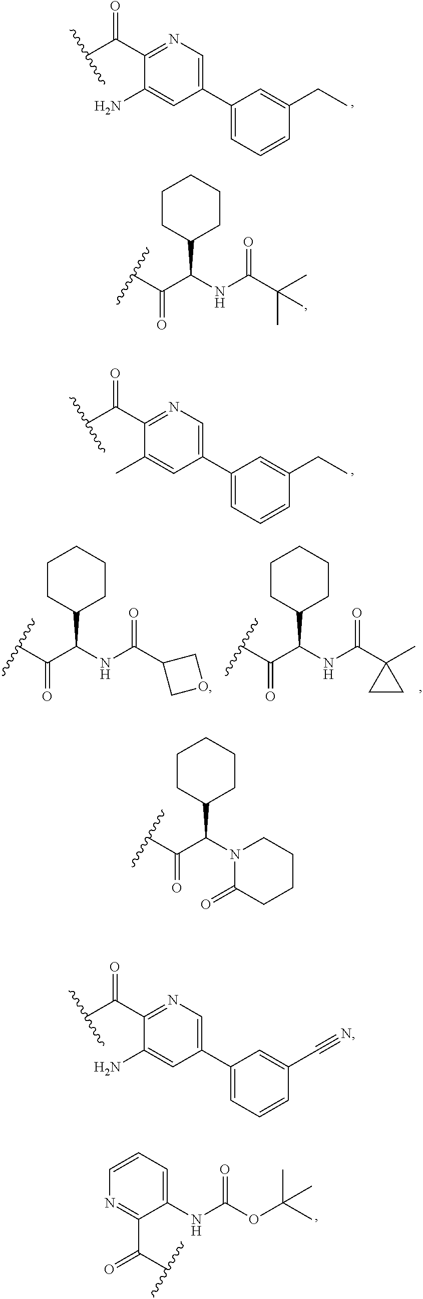

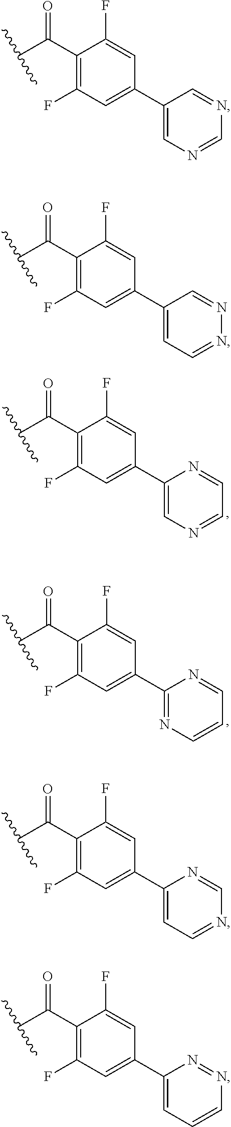

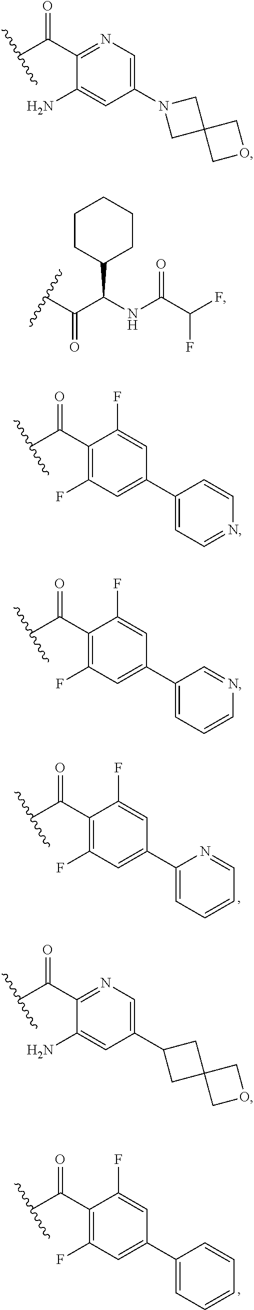

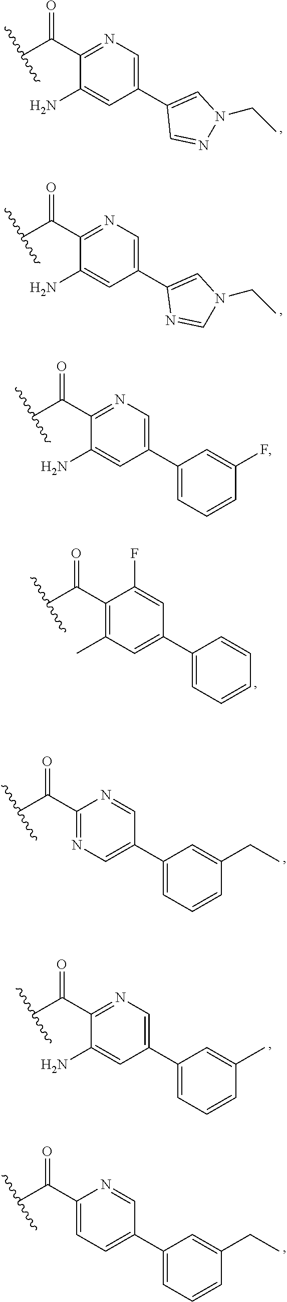

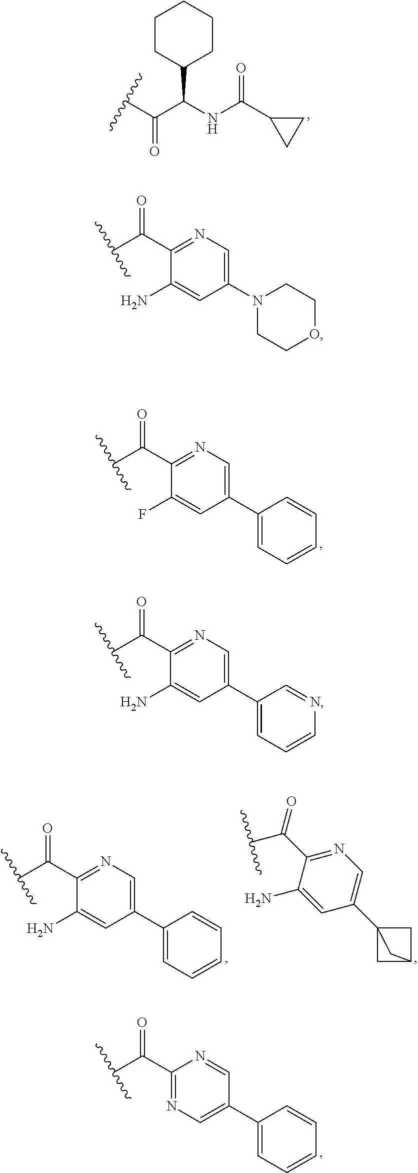

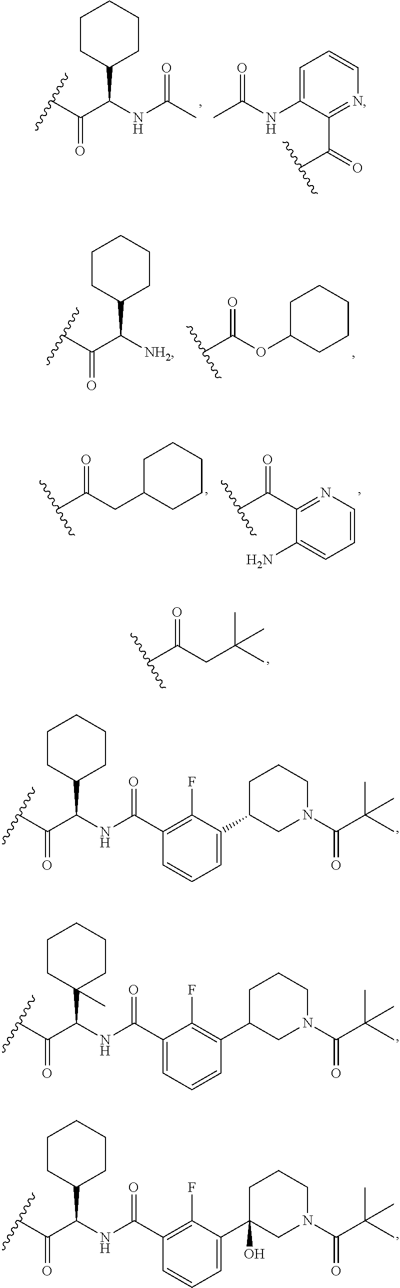

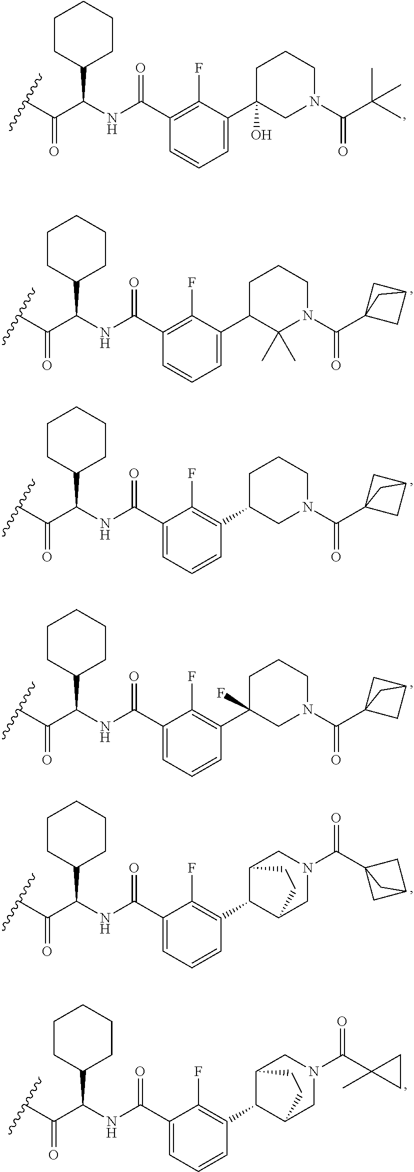

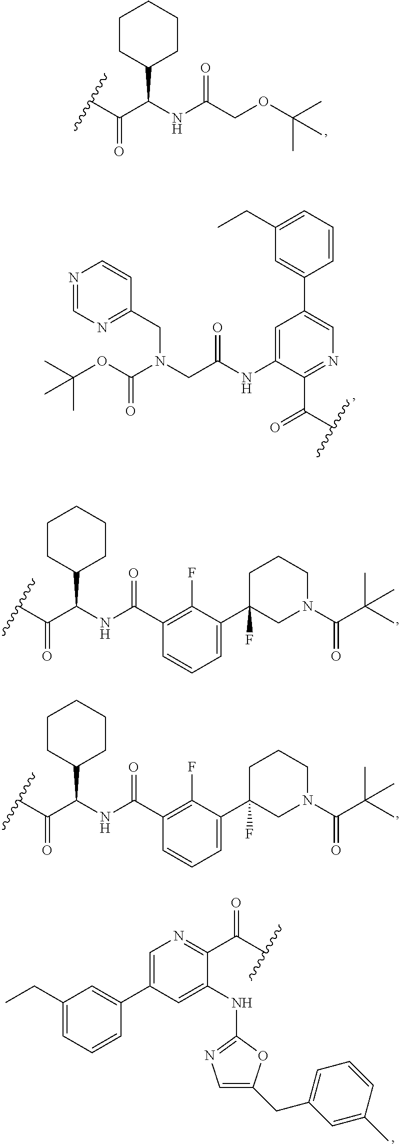

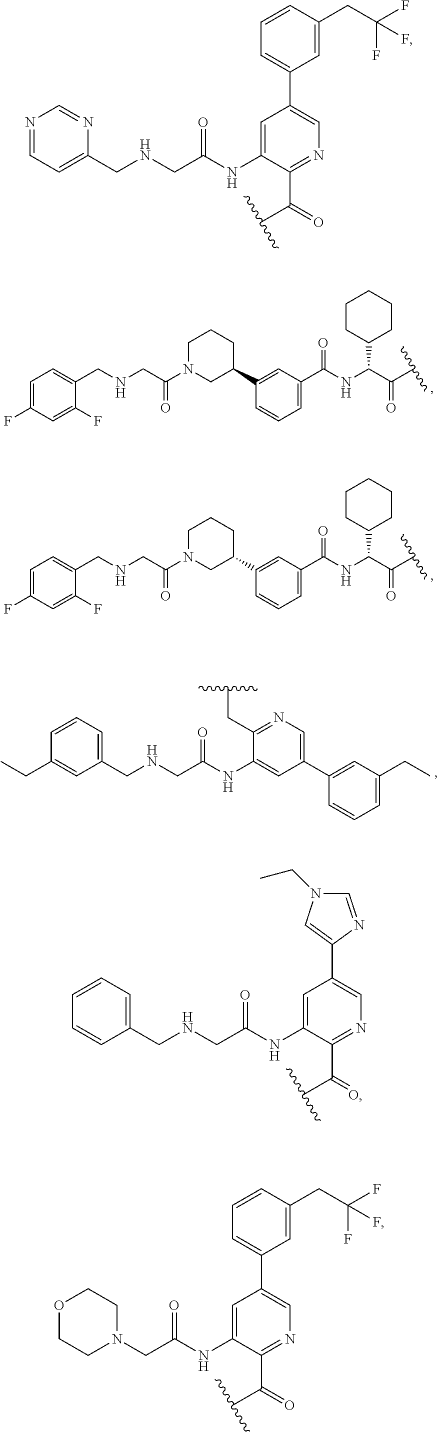

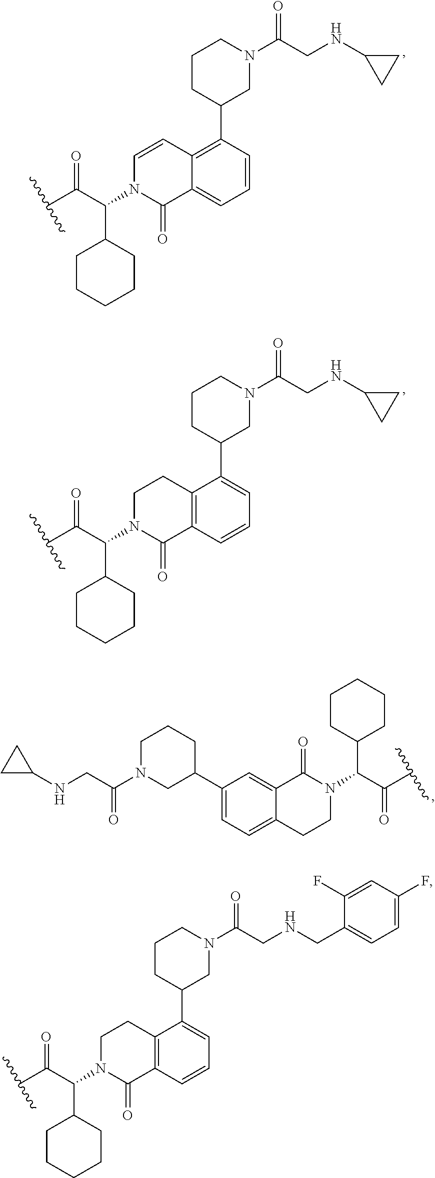

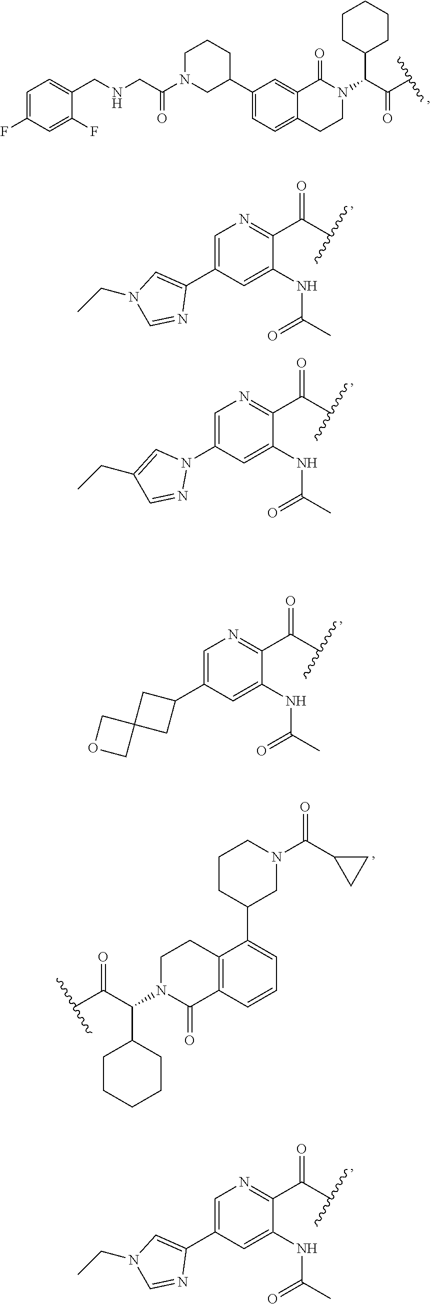

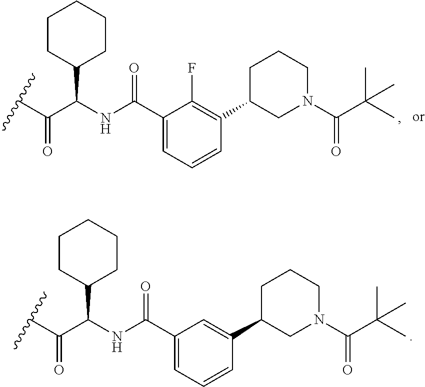 [SMILES: *C(=O)[C@H](NC(=O)c1cccc([C@@H]2CCCN(C(=O)C(C)(C)C)C2)c1F)C1CCCCC1 ; "," ; "or" ; *C(=O)[C@H](NC(=O)c1cccc([C@H]2CCCN(C(=O)C(C)(C)C)C2)c1)C1CCCCC1 ; "."]

In some embodiments, DBM is

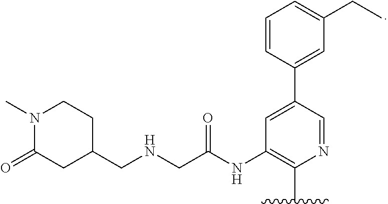 [SMILES: *c1ncc(-c2cccc(CC)c2)cc1NC(=O)CNCC1CCN(C)C(=O)C1]

In some embodiments, DBM is

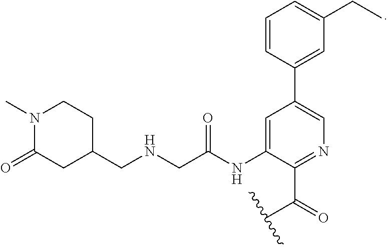 [SMILES: *C(=O)c1ncc(-c2cccc(CC)c2)cc1NC(=O)CNCC1CCN(C)C(=O)C1]

In some embodiments, DBM is

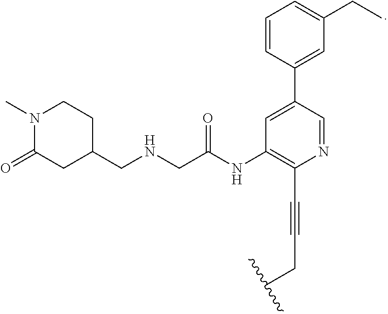 [SMILES: *CC#Cc1ncc(-c2cccc(CC)c2)cc1NC(=O)CNCC1CCN(C)C(=O)C1]

In some embodiments, DBM is

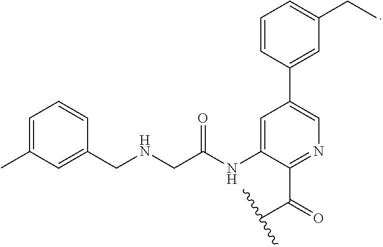 [SMILES: *C(=O)c1ncc(-c2cccc(CC)c2)cc1NC(=O)CNCc1cccc(C)c1]

In some embodiments, DBM is

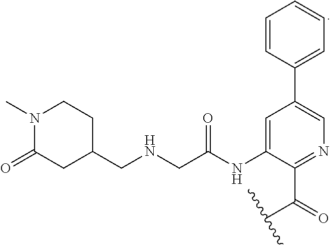 [SMILES: *C(=O)c1ncc(-c2ccccc2)cc1NC(=O)CNCC1CCN(C)C(=O)C1]

In some embodiments, DBM is

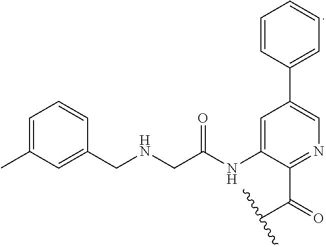 [SMILES: *C(=O)c1ncc(-c2ccccc2)cc1NC(=O)CNCc1cccc(C)c1]

In some embodiments, DBM is

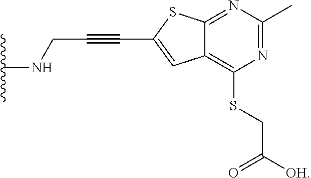 [SMILES: *NCC#Cc1cc2c(SCC(=O)O)nc(C)nc2s1]

In some embodiments, DBM is

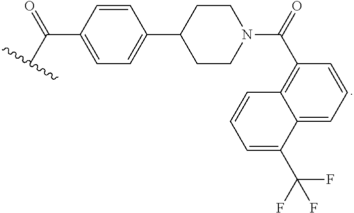 [SMILES: *C(=O)c1ccc(C2CCN(C(=O)c3cccc4c(C(F)(F)F)cccc34)CC2)cc1]

In some embodiments, DBM is

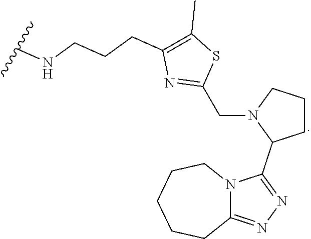 [SMILES: *NCCCc1nc(CN2CCCC2c2nnc3n2CCCCC3)sc1C]

In some embodiments, DBM is

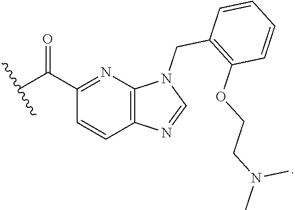 [SMILES: *C(=O)c1ccc2ncn(Cc3ccccc3OCCN(C)C)c2n1]

In some embodiments, DBM is

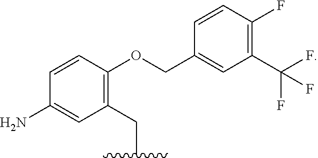 [SMILES: *Cc1cc(N)ccc1OCc1ccc(F)c(C(F)(F)F)c1]

In some embodiments, DBM is

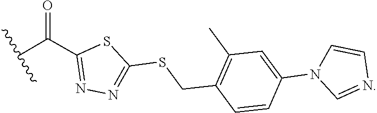 [SMILES: *C(=O)c1nnc(SCc2ccc(-n3ccnc3)cc2C)s1]

In some embodiments, DBM is

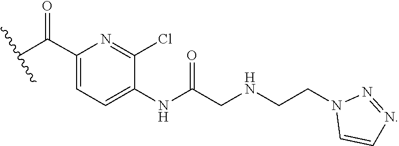 [SMILES: *C(=O)c1ccc(NC(=O)CNCCn2ccnn2)c(Cl)n1]

In some embodiments, DBM is

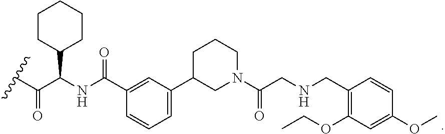 [SMILES: *C(=O)[C@H](NC(=O)c1cccc(C2CCCN(C(=O)CNCc3ccc(OC)cc3OCC)C2)c1)C1CCCCC1]

In some embodiments, DBM is

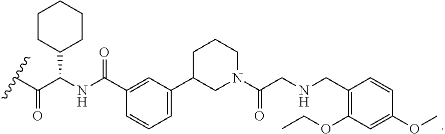 [SMILES: *C(=O)[C@@H](NC(=O)c1cccc(C2CCCN(C(=O)CNCc3ccc(OC)cc3OCC)C2)c1)C1CCCCC1]

In some embodiments, DBM is

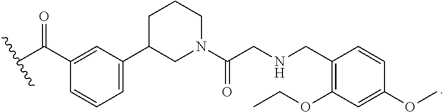 [SMILES: *C(=O)c1cccc(C2CCCN(C(=O)CNCc3ccc(OC)cc3OCC)C2)c1]

In some embodiments, DBM is

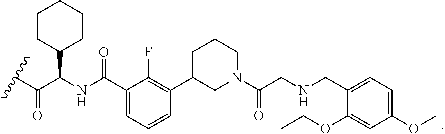 [SMILES: *C(=O)[C@H](NC(=O)c1cccc(C2CCCN(C(=O)CNCc3ccc(OC)cc3OCC)C2)c1F)C1CCCCC1]

In some embodiments, DBM is

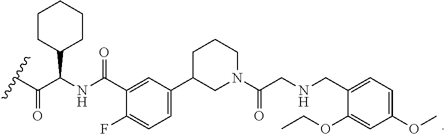 [SMILES: *C(=O)[C@H](NC(=O)c1cc(C2CCCN(C(=O)CNCc3ccc(OC)cc3OCC)C2)ccc1F)C1CCCCC1]

In some embodiments, DBM is

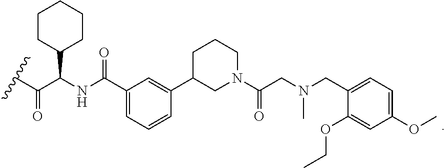 [SMILES: *C(=O)[C@H](NC(=O)c1cccc(C2CCCN(C(=O)CN(C)Cc3ccc(OC)cc3OCC)C2)c1)C1CCCCC1]

In some embodiments, DBM is

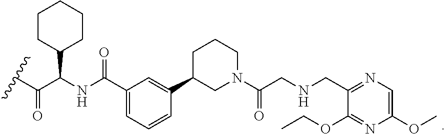 [SMILES: *C(=O)[C@H](NC(=O)c1cccc([C@H]2CCCN(C(=O)CNCc3ncc(OC)nc3OCC)C2)c1)C1CCCCC1]

In some embodiments, DBM is

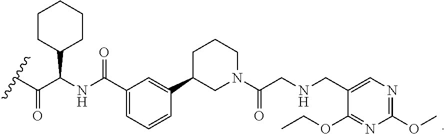 [SMILES: *C(=O)[C@H](NC(=O)c1cccc([C@H]2CCCN(C(=O)CNCc3cnc(OC)nc3OCC)C2)c1)C1CCCCC1]

In some embodiments, DBM is

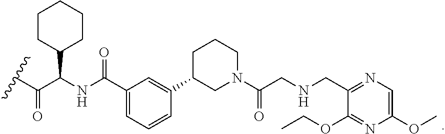 [SMILES: *C(=O)[C@H](NC(=O)c1cccc([C@@H]2CCCN(C(=O)CNCc3ncc(OC)nc3OCC)C2)c1)C1CCCCC1]

In some embodiments, DBM is

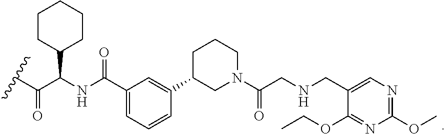 [SMILES: *C(=O)[C@H](NC(=O)c1cccc([C@@H]2CCCN(C(=O)CNCc3cnc(OC)nc3OCC)C2)c1)C1CCCCC1]

In some embodiments, DBM is

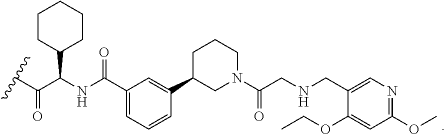 [SMILES: *C(=O)[C@H](NC(=O)c1cccc([C@H]2CCCN(C(=O)CNCc3cnc(OC)cc3OCC)C2)c1)C1CCCCC1]

In some embodiments, DBM is

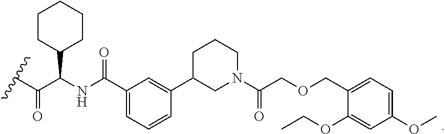 [SMILES: *C(=O)[C@H](NC(=O)c1cccc(C2CCCN(C(=O)COCc3ccc(OC)cc3OCC)C2)c1)C1CCCCC1]

In some embodiments, DBM is

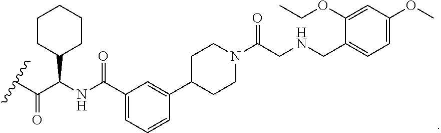 [SMILES: *C(=O)[C@H](NC(=O)c1cccc(C2CCN(C(=O)CNCc3ccc(OC)cc3OCC)CC2)c1)C1CCCCC1]

In some embodiments, DBM is

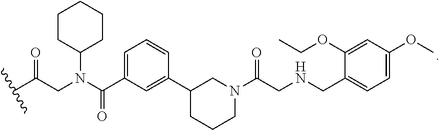 [SMILES: *C(=O)CN(C(=O)c1cccc(C2CCCN(C(=O)CNCc3ccc(OC)cc3OCC)C2)c1)C1CCCCC1]

In some embodiments, DBM is

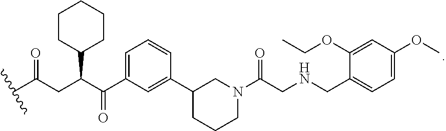 [SMILES: *C(=O)C[C@H](C(=O)c1cccc(C2CCCN(C(=O)CNCc3ccc(OC)cc3OCC)C2)c1)C1CCCCC1]

In some embodiments, DBM is

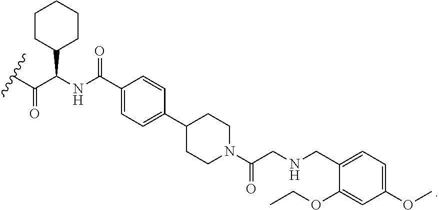 [SMILES: *C(=O)[C@H](NC(=O)c1ccc(C2CCN(C(=O)CNCc3ccc(OC)cc3OCC)CC2)cc1)C1CCCCC1]

In some embodiments, DBM is

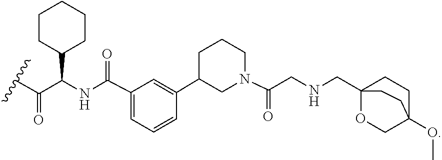 [SMILES: *C(=O)[C@H](NC(=O)c1cccc(C2CCCN(C(=O)CNCC34CCC(OC)(CC3)CO4)C2)c1)C1CCCCC1]

In some embodiments, DBM is

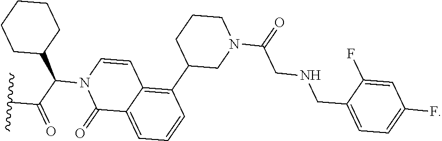 [SMILES: *C(=O)[C@@H](C1CCCCC1)n1ccc2c(C3CCCN(C(=O)CNCc4ccc(F)cc4F)C3)cccc2c1=O]

In some embodiments, DBM is

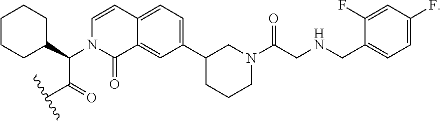 [SMILES: *C(=O)[C@@H](C1CCCCC1)n1ccc2ccc(C3CCCN(C(=O)CNCc4ccc(F)cc4F)C3)cc2c1=O]

In some embodiments, DBM is

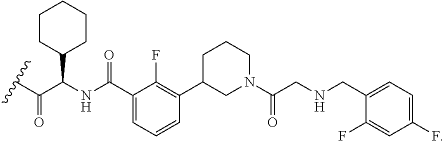 [SMILES: *C(=O)[C@H](NC(=O)c1cccc(C2CCCN(C(=O)CNCc3ccc(F)cc3F)C2)c1F)C1CCCCC1]

In some embodiments, DBM is

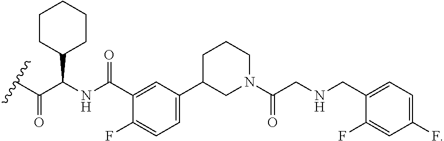 [SMILES: *C(=O)[C@H](NC(=O)c1cc(C2CCCN(C(=O)CNCc3ccc(F)cc3F)C2)ccc1F)C1CCCCC1]

In some embodiments, DBM is

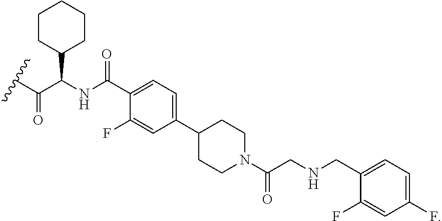 [SMILES: *C(=O)[C@H](NC(=O)c1ccc(C2CCN(C(=O)CNCc3ccc(F)cc3F)CC2)cc1F)C1CCCCC1]

In some embodiments, DBM is

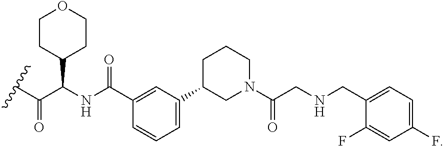 [SMILES: *C(=O)[C@H](NC(=O)c1cccc([C@@H]2CCCN(C(=O)CNCc3ccc(F)cc3F)C2)c1)C1CCOCC1]

In some embodiments, DBM is

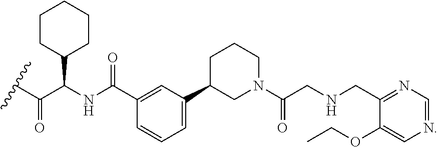 [SMILES: *C(=O)[C@H](NC(=O)c1cccc([C@H]2CCCN(C(=O)CNCc3ncncc3OCC)C2)c1)C1CCCCC1]

In some embodiments, DBM is

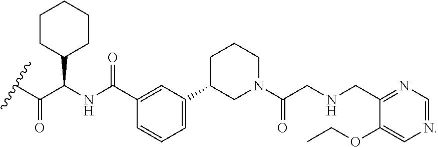 [SMILES: *C(=O)[C@H](NC(=O)c1cccc([C@@H]2CCCN(C(=O)CNCc3ncncc3OCC)C2)c1)C1CCCCC1]

In some embodiments, DBM is

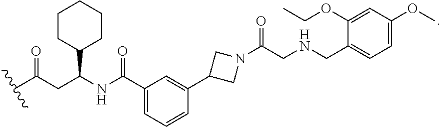 [SMILES: *C(=O)C[C@H](NC(=O)c1cccc(C2CN(C(=O)CNCc3ccc(OC)cc3OCC)C2)c1)C1CCCCC1]

In some embodiments, DBM is

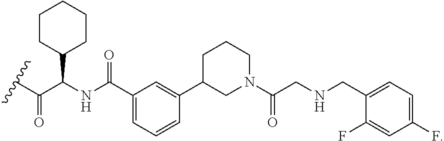 [SMILES: *C(=O)[C@H](NC(=O)c1cccc(C2CCCN(C(=O)CNCc3ccc(F)cc3F)C2)c1)C1CCCCC1]

In some embodiments, DBM is

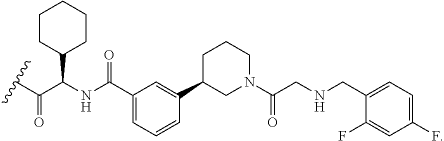 [SMILES: *C(=O)[C@H](NC(=O)c1cccc([C@H]2CCCN(C(=O)CNCc3ccc(F)cc3F)C2)c1)C1CCCCC1]

In some embodiments, DBM is

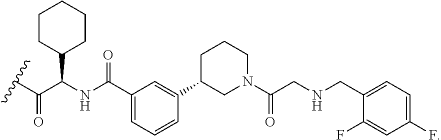 [SMILES: *C(=O)[C@H](NC(=O)c1cccc([C@@H]2CCCN(C(=O)CNCc3ccc(F)cc3F)C2)c1)C1CCCCC1]

In some embodiments, DBM is

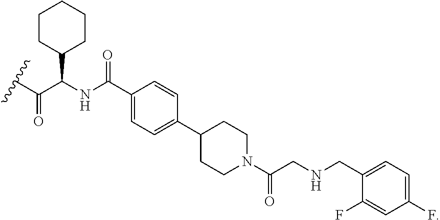 [SMILES: *C(=O)[C@H](NC(=O)c1ccc(C2CCN(C(=O)CNCc3ccc(F)cc3F)CC2)cc1)C1CCCCC1]

In some embodiments, DBM is

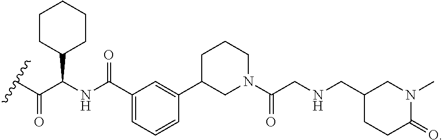 [SMILES: *C(=O)[C@H](NC(=O)c1cccc(C2CCCN(C(=O)CNCC3CCC(=O)N(C)C3)C2)c1)C1CCCCC1]

In some embodiments, DBM is

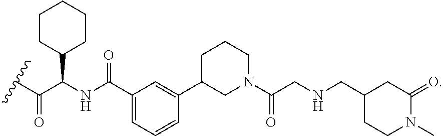 [SMILES: *C(=O)[C@H](NC(=O)c1cccc(C2CCCN(C(=O)CNCC3CCN(C)C(=O)C3)C2)c1)C1CCCCC1]

In some embodiments, DBM is

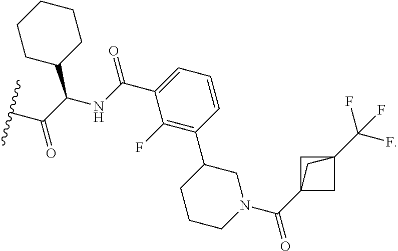 [SMILES: *C(=O)[C@H](NC(=O)c1cccc(C2CCCN(C(=O)C34CC(C(F)(F)F)(C3)C4)C2)c1F)C1CCCCC1]

In some embodiments, DBM is

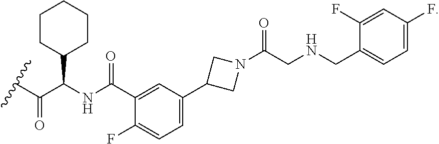 [SMILES: *C(=O)[C@H](NC(=O)c1cc(C2CN(C(=O)CNCc3ccc(F)cc3F)C2)ccc1F)C1CCCCC1]

In some embodiments, DBM is

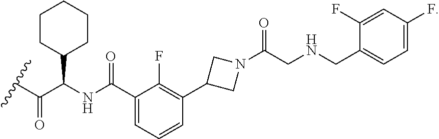 [SMILES: *C(=O)[C@H](NC(=O)c1cccc(C2CN(C(=O)CNCc3ccc(F)cc3F)C2)c1F)C1CCCCC1]

In some embodiments, DBM is

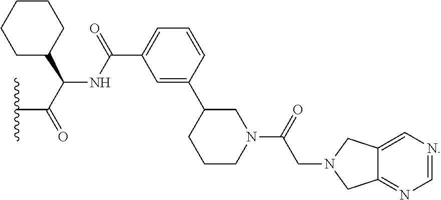 [SMILES: *C(=O)[C@H](NC(=O)c1cccc(C2CCCN(C(=O)CN3Cc4cncnc4C3)C2)c1)C1CCCCC1]

In some embodiments, DBM is

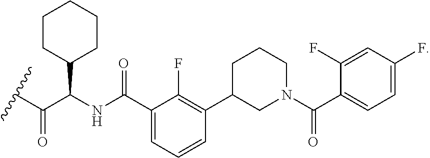 [SMILES: *C(=O)[C@H](NC(=O)c1cccc(C2CCCN(C(=O)c3ccc(F)cc3F)C2)c1F)C1CCCCC1]

In some embodiments, DBM is

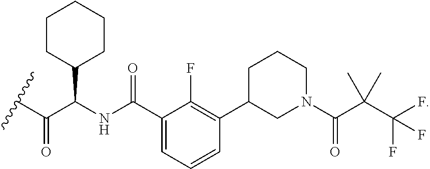 [SMILES: *C(=O)[C@H](NC(=O)c1cccc(C2CCCN(C(=O)C(C)(C)C(F)(F)F)C2)c1F)C1CCCCC1]

In some embodiments, DBM is

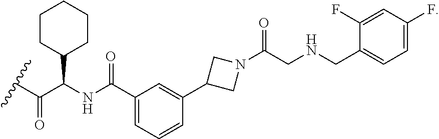 [SMILES: *C(=O)[C@H](NC(=O)c1cccc(C2CN(C(=O)CNCc3ccc(F)cc3F)C2)c1)C1CCCCC1]

In some embodiments, DBM is

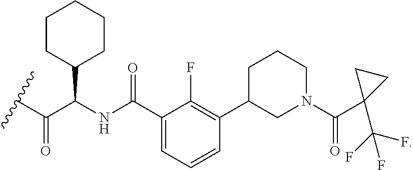 [SMILES: *C(=O)[C@H](NC(=O)c1cccc(C2CCCN(C(=O)C3(C(F)(F)F)CC3)C2)c1F)C1CCCCC1]

In some embodiments, DBM is

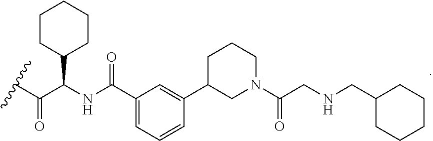 [SMILES: *C(=O)[C@H](NC(=O)c1cccc(C2CCCN(C(=O)CNCC3CCCCC3)C2)c1)C1CCCCC1]

In some embodiments, DBM is

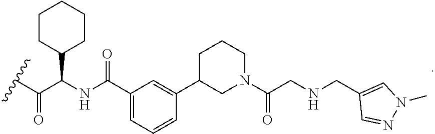 [SMILES: *C(=O)[C@H](NC(=O)c1cccc(C2CCCN(C(=O)CNCc3cnn(C)c3)C2)c1)C1CCCCC1]

In some embodiments, DBM is

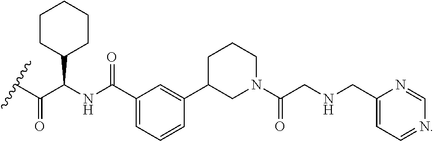 [SMILES: *C(=O)[C@H](NC(=O)c1cccc(C2CCCN(C(=O)CNCc3ccncn3)C2)c1)C1CCCCC1]

In some embodiments, DBM is

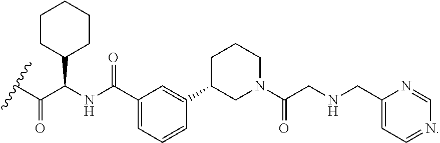 [SMILES: *C(=O)[C@H](NC(=O)c1cccc([C@@H]2CCCN(C(=O)CNCc3ccncn3)C2)c1)C1CCCCC1]

In some embodiments, DBM is

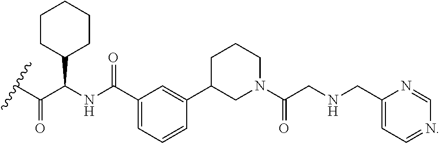 [SMILES: *C(=O)[C@H](NC(=O)c1cccc(C2CCCN(C(=O)CNCc3ccncn3)C2)c1)C1CCCCC1]

In some embodiments, DBM is

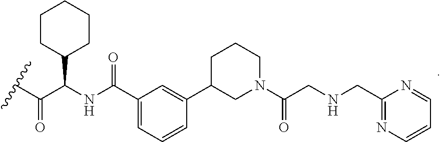 [SMILES: *C(=O)[C@H](NC(=O)c1cccc(C2CCCN(C(=O)CNCc3ncccn3)C2)c1)C1CCCCC1]

In some embodiments, DBM is

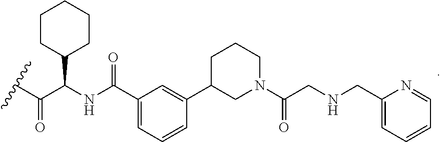 [SMILES: *C(=O)[C@H](NC(=O)c1cccc(C2CCCN(C(=O)CNCc3ccccn3)C2)c1)C1CCCCC1]

In some embodiments, DBM is

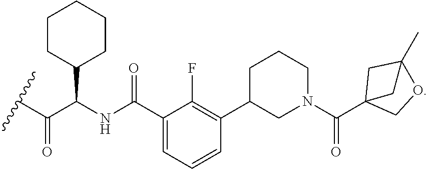 [SMILES: *C(=O)[C@H](NC(=O)c1cccc(C2CCCN(C(=O)C34COC(C)(C3)C4)C2)c1F)C1CCCCC1]

In some embodiments, DBM is

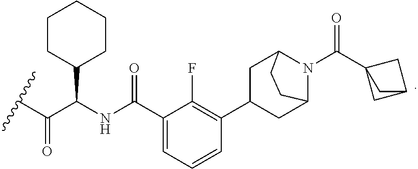 [SMILES: *C(=O)[C@H](NC(=O)c1cccc(C2CC3CCC(C2)N3C(=O)C23CC(C2)C3)c1F)C1CCCCC1]

In some embodiments, DBM is

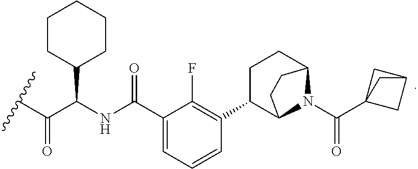 [SMILES: *C(=O)[C@H](NC(=O)c1cccc([C@@H]2CC[C@@H]3CC[C@H]2N3C(=O)C23CC(C2)C3)c1F)C1CCCCC1]

In some embodiments, DBM is

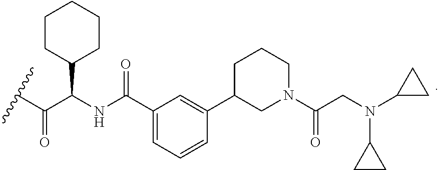 [SMILES: *C(=O)[C@H](NC(=O)c1cccc(C2CCCN(C(=O)CN(C3CC3)C3CC3)C2)c1)C1CCCCC1]

In some embodiments, DBM is

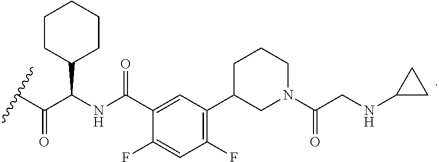 [SMILES: *C(=O)[C@H](NC(=O)c1cc(C2CCCN(C(=O)CNC3CC3)C2)c(F)cc1F)C1CCCCC1]

In some embodiments, DBM is

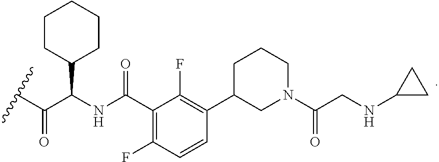 [SMILES: *C(=O)[C@H](NC(=O)c1c(F)ccc(C2CCCN(C(=O)CNC3CC3)C2)c1F)C1CCCCC1]

In some embodiments, DBM is

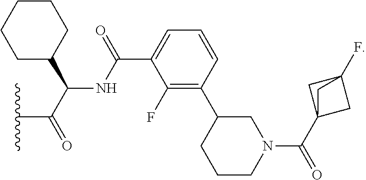 [SMILES: *C(=O)[C@H](NC(=O)c1cccc(C2CCCN(C(=O)C34CC(F)(C3)C4)C2)c1F)C1CCCCC1]

In some embodiments, DBM is

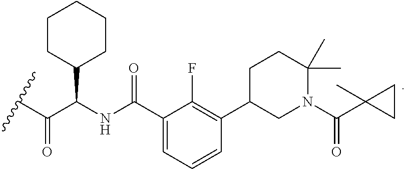 [SMILES: *C(=O)[C@H](NC(=O)c1cccc(C2CCC(C)(C)N(C(=O)C3(C)CC3)C2)c1F)C1CCCCC1]

In some embodiments, DBM is

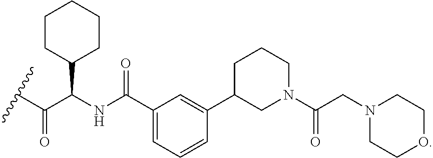 [SMILES: *C(=O)[C@H](NC(=O)c1cccc(C2CCCN(C(=O)CN3CCOCC3)C2)c1)C1CCCCC1]

In some embodiments, DBM is

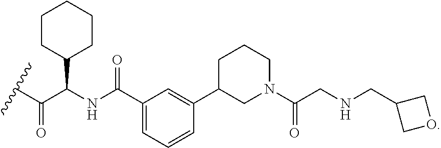 [SMILES: *C(=O)[C@H](NC(=O)c1cccc(C2CCCN(C(=O)CNCC3COC3)C2)c1)C1CCCCC1]

In some embodiments, DBM is

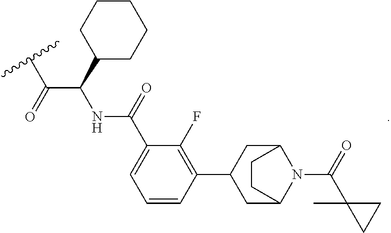 [SMILES: *C(=O)[C@H](NC(=O)c1cccc(C2CC3CCC(C2)N3C(=O)C2(C)CC2)c1F)C1CCCCC1]

In some embodiments, DBM is

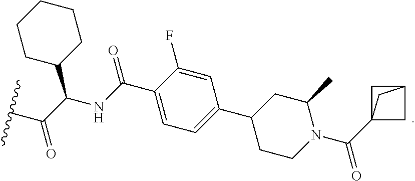 [SMILES: *C(=O)[C@H](NC(=O)c1ccc(C2CCN(C(=O)C34CC(C3)C4)[C@H](C)C2)cc1F)C1CCCCC1]

In some embodiments, DBM is

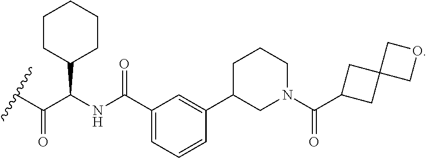 [SMILES: *C(=O)[C@H](NC(=O)c1cccc(C2CCCN(C(=O)C3CC4(COC4)C3)C2)c1)C1CCCCC1]

In some embodiments, DBM is

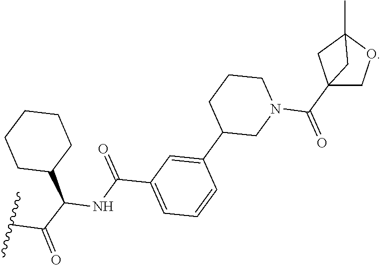 [SMILES: *C(=O)[C@H](NC(=O)c1cccc(C2CCCN(C(=O)C34COC(C)(C3)C4)C2)c1)C1CCCCC1]

In some embodiments, DBM is

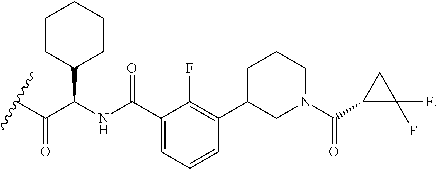 [SMILES: *C(=O)[C@H](NC(=O)c1cccc(C2CCCN(C(=O)[C@@H]3CC3(F)F)C2)c1F)C1CCCCC1]

In some embodiments, DBM is

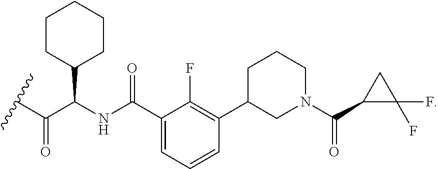 [SMILES: *C(=O)[C@H](NC(=O)c1cccc(C2CCCN(C(=O)[C@H]3CC3(F)F)C2)c1F)C1CCCCC1]

In some embodiments, DBM is

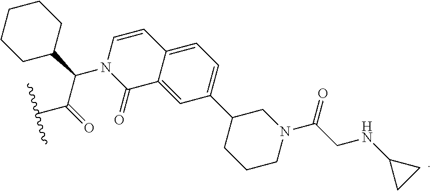 [SMILES: *C(=O)[C@@H](C1CCCCC1)n1ccc2ccc(C3CCCN(C(=O)CNC4CC4)C3)cc2c1=O]

In some embodiments, DBM is

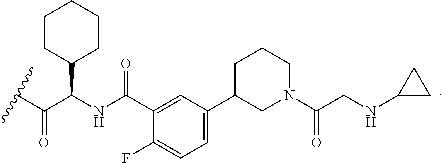 [SMILES: *C(=O)[C@H](NC(=O)c1cc(C2CCCN(C(=O)CNC3CC3)C2)ccc1F)C1CCCCC1]

In some embodiments, DBM is

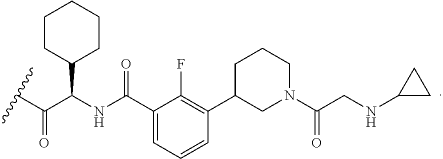 [SMILES: *C(=O)[C@H](NC(=O)c1cccc(C2CCCN(C(=O)CNC3CC3)C2)c1F)C1CCCCC1]

In some embodiments, DBM is

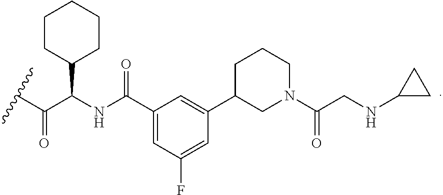 [SMILES: *C(=O)[C@H](NC(=O)c1cc(F)cc(C2CCCN(C(=O)CNC3CC3)C2)c1)C1CCCCC1]

In some embodiments, DBM is

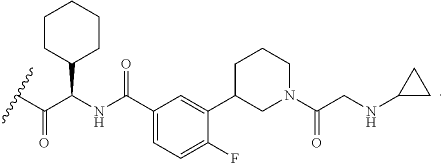 [SMILES: *C(=O)[C@H](NC(=O)c1ccc(F)c(C2CCCN(C(=O)CNC3CC3)C2)c1)C1CCCCC1]

In some embodiments, DBM is

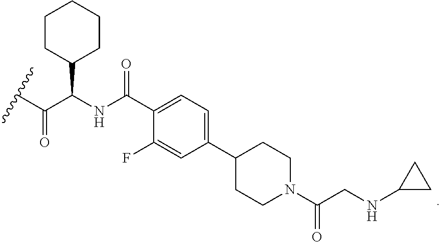 [SMILES: *C(=O)[C@H](NC(=O)c1ccc(C2CCN(C(=O)CNC3CC3)CC2)cc1F)C1CCCCC1]

In some embodiments, DBM is

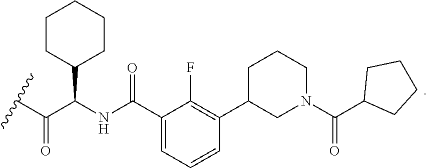 [SMILES: *C(=O)[C@H](NC(=O)c1cccc(C2CCCN(C(=O)C3CCCC3)C2)c1F)C1CCCCC1]

In some embodiments, DBM is

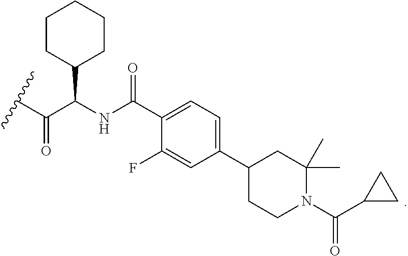 [SMILES: *C(=O)[C@H](NC(=O)c1ccc(C2CCN(C(=O)C3CC3)C(C)(C)C2)cc1F)C1CCCCC1]

In some embodiments, DBM is

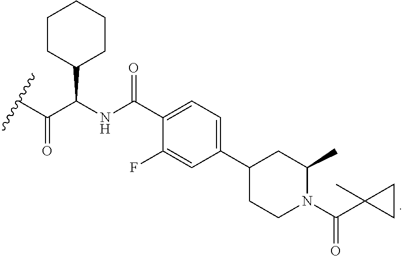 [SMILES: *C(=O)[C@H](NC(=O)c1ccc(C2CCN(C(=O)C3(C)CC3)[C@H](C)C2)cc1F)C1CCCCC1]

In some embodiments, DBM is

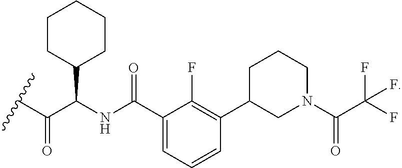 [SMILES: *C(=O)[C@H](NC(=O)c1cccc(C2CCCN(C(=O)C(F)(F)F)C2)c1F)C1CCCCC1]

In some embodiments, DBM is

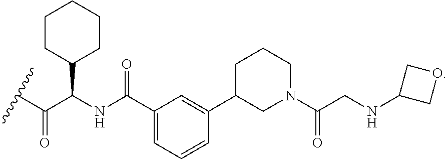 [SMILES: *C(=O)[C@H](NC(=O)c1cccc(C2CCCN(C(=O)CNC3COC3)C2)c1)C1CCCCC1]

In some embodiments, DBM is

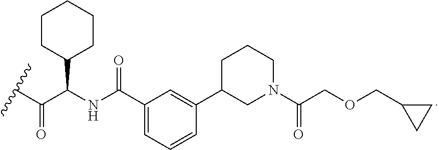 [SMILES: *C(=O)[C@H](NC(=O)c1cccc(C2CCCN(C(=O)COCC3CC3)C2)c1)C1CCCCC1]

In some embodiments, DBM is

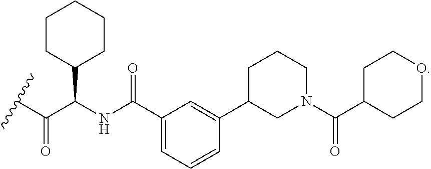 [SMILES: *C(=O)[C@H](NC(=O)c1cccc(C2CCCN(C(=O)C3CCOCC3)C2)c1)C1CCCCC1]

In some embodiments, DBM is

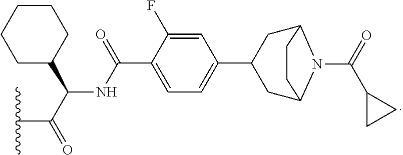 [SMILES: *C(=O)[C@H](NC(=O)c1ccc(C2CC3CCC(C2)N3C(=O)C2CC2)cc1F)C1CCCCC1]

In some embodiments, DBM is

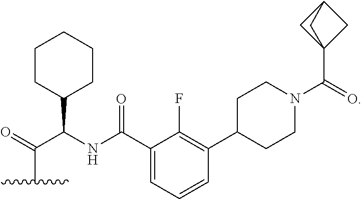 [SMILES: *C(=O)[C@H](NC(=O)c1cccc(C2CCN(C(=O)C34CC(C3)C4)CC2)c1F)C1CCCCC1]

In some embodiments, DBM is

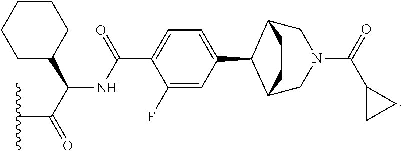 [SMILES: *C(=O)[C@H](NC(=O)c1ccc([C@@H]2[C@@H]3CC[C@H]2CN(C(=O)C2CC2)C3)cc1F)C1CCCCC1]

In some embodiments, DBM is

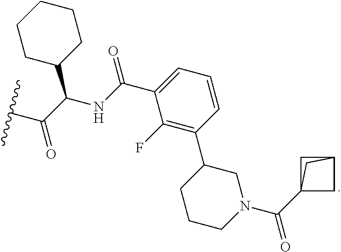 [SMILES: *C(=O)[C@H](NC(=O)c1cccc(C2CCCN(C(=O)C34CC(C3)C4)C2)c1F)C1CCCCC1]

In some embodiments, DBM is

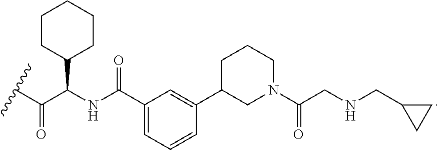 [SMILES: *C(=O)[C@H](NC(=O)c1cccc(C2CCCN(C(=O)CNCC3CC3)C2)c1)C1CCCCC1]

In some embodiments, DBM is

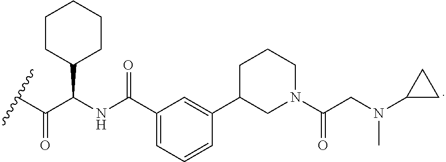 [SMILES: *C(=O)[C@H](NC(=O)c1cccc(C2CCCN(C(=O)CN(C)C3CC3)C2)c1)C1CCCCC1]

In some embodiments, DBM is

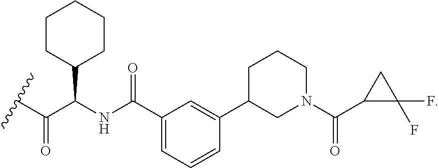 [SMILES: *C(=O)[C@H](NC(=O)c1cccc(C2CCCN(C(=O)C3CC3(F)F)C2)c1)C1CCCCC1]

In some embodiments, DBM is

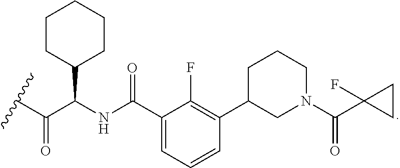 [SMILES: *C(=O)[C@H](NC(=O)c1cccc(C2CCCN(C(=O)C3(F)CC3)C2)c1F)C1CCCCC1]

In some embodiments, DBM is

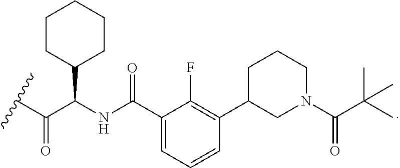 [SMILES: *C(=O)[C@H](NC(=O)c1cccc(C2CCCN(C(=O)C(C)(C)C)C2)c1F)C1CCCCC1]

In some embodiments, DBM is

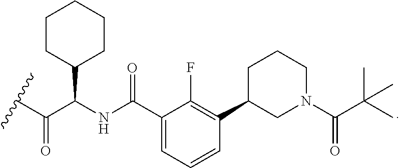 [SMILES: *C(=O)[C@H](NC(=O)c1cccc([C@H]2CCCN(C(=O)C(C)(C)C)C2)c1F)C1CCCCC1]

In some embodiments, DBM is

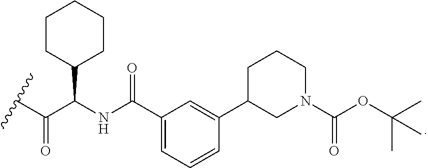 [SMILES: *C(=O)[C@H](NC(=O)c1cccc(C2CCCN(C(=O)OC(C)(C)C)C2)c1)C1CCCCC1]

In some embodiments, DBM is

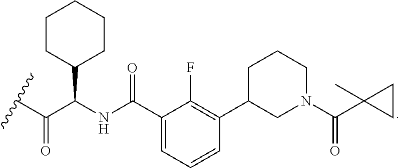 [SMILES: *C(=O)[C@H](NC(=O)c1cccc(C2CCCN(C(=O)C3(C)CC3)C2)c1F)C1CCCCC1]

In some embodiments, DBM is

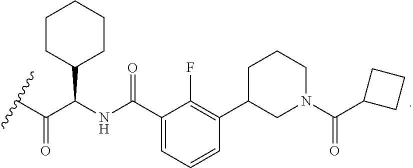 [SMILES: *C(=O)[C@H](NC(=O)c1cccc(C2CCCN(C(=O)C3CCC3)C2)c1F)C1CCCCC1]

In some embodiments, DBM is

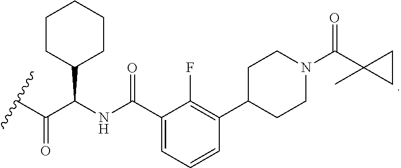 [SMILES: *C(=O)[C@H](NC(=O)c1cccc(C2CCN(C(=O)C3(C)CC3)CC2)c1F)C1CCCCC1]

In some embodiments, DBM is

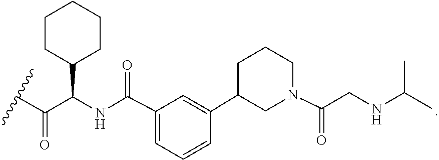 [SMILES: *C(=O)[C@H](NC(=O)c1cccc(C2CCCN(C(=O)CNC(C)C)C2)c1)C1CCCCC1]

In some embodiments, DBM is

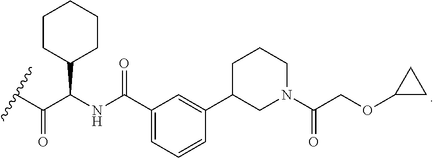 [SMILES: *C(=O)[C@H](NC(=O)c1cccc(C2CCCN(C(=O)COC3CC3)C2)c1)C1CCCCC1]

In some embodiments, DBM is

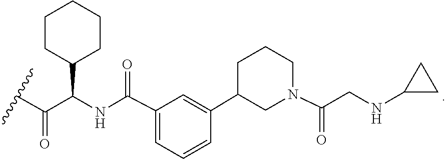 [SMILES: *C(=O)[C@H](NC(=O)c1cccc(C2CCCN(C(=O)CNC3CC3)C2)c1)C1CCCCC1]

In some embodiments, DBM is

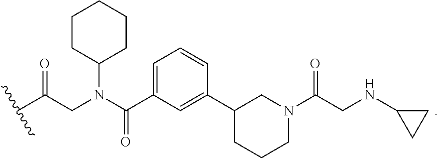 [SMILES: *C(=O)CN(C(=O)c1cccc(C2CCCN(C(=O)CNC3CC3)C2)c1)C1CCCCC1]

In some embodiments, DBM is

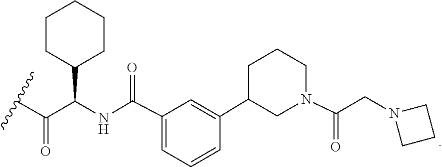 [SMILES: *C(=O)[C@H](NC(=O)c1cccc(C2CCCN(C(=O)CN3CCC3)C2)c1)C1CCCCC1]

In some embodiments, DBM is

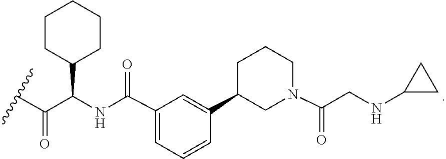 [SMILES: *C(=O)[C@H](NC(=O)c1cccc([C@H]2CCCN(C(=O)CNC3CC3)C2)c1)C1CCCCC1]

In some embodiments, DBM is

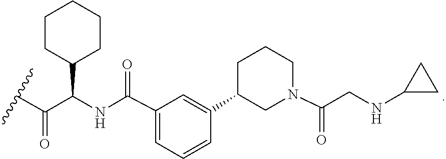 [SMILES: *C(=O)[C@H](NC(=O)c1cccc([C@@H]2CCCN(C(=O)CNC3CC3)C2)c1)C1CCCCC1]

In some embodiments, DBM is

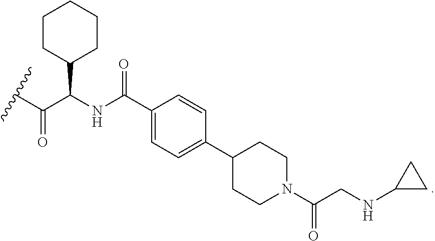 [SMILES: *C(=O)[C@H](NC(=O)c1ccc(C2CCN(C(=O)CNC3CC3)CC2)cc1)C1CCCCC1]

In some embodiments, DBM is

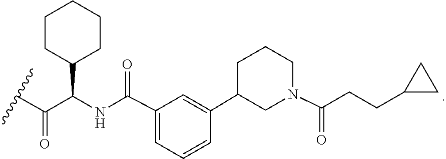 [SMILES: *C(=O)[C@H](NC(=O)c1cccc(C2CCCN(C(=O)CCC3CC3)C2)c1)C1CCCCC1]

In some embodiments, DBM is

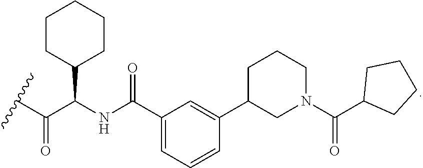 [SMILES: *C(=O)[C@H](NC(=O)c1cccc(C2CCCN(C(=O)C3CCCC3)C2)c1)C1CCCCC1]

In some embodiments, DBM is

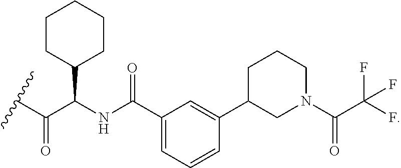 [SMILES: *C(=O)[C@H](NC(=O)c1cccc(C2CCCN(C(=O)C(F)(F)F)C2)c1)C1CCCCC1]

In some embodiments, DBM is

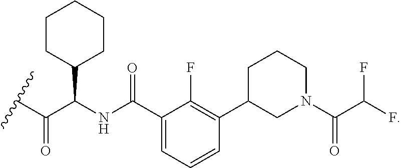 [SMILES: *C(=O)[C@H](NC(=O)c1cccc(C2CCCN(C(=O)C(F)F)C2)c1F)C1CCCCC1]

In some embodiments, DBM is

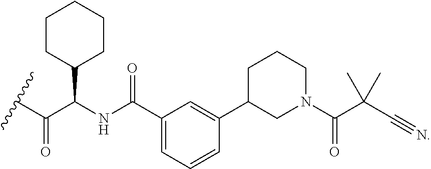 [SMILES: *C(=O)[C@H](NC(=O)c1cccc(C2CCCN(C(=O)C(C)(C)C#N)C2)c1)C1CCCCC1]

In some embodiments, DBM is

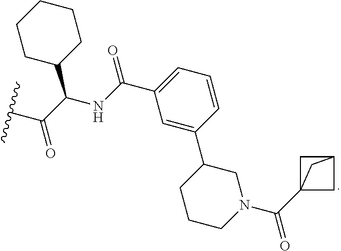 [SMILES: *C(=O)[C@H](NC(=O)c1cccc(C2CCCN(C(=O)C34CC(C3)C4)C2)c1)C1CCCCC1]

In some embodiments, DBM is

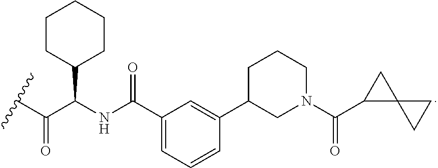 [SMILES: *C(=O)[C@H](NC(=O)c1cccc(C2CCCN(C(=O)C3CC34CC4)C2)c1)C1CCCCC1]

In some embodiments, DBM is

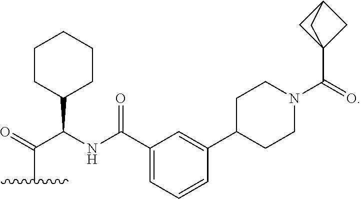 [SMILES: *C(=O)[C@H](NC(=O)c1cccc(C2CCN(C(=O)C34CC(C3)C4)CC2)c1)C1CCCCC1]

In some embodiments, DBM is

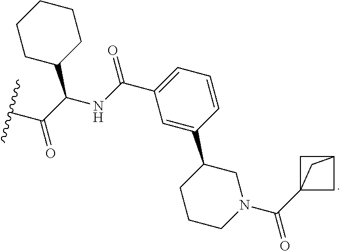 [SMILES: *C(=O)[C@H](NC(=O)c1cccc([C@@H]2CCCN(C(=O)C34CC(C3)C4)C2)c1)C1CCCCC1]

In some embodiments, DBM is

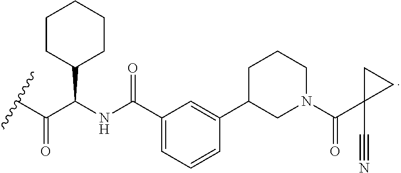 [SMILES: *C(=O)[C@H](NC(=O)c1cccc(C2CCCN(C(=O)C3(C#N)CC3)C2)c1)C1CCCCC1]

In some embodiments, DBM is

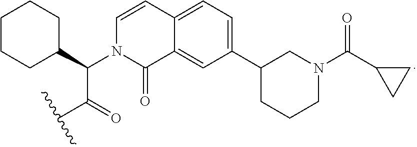 [SMILES: *C(=O)[C@@H](C1CCCCC1)n1ccc2ccc(C3CCCN(C(=O)C4CC4)C3)cc2c1=O]

In some embodiments, DBM is

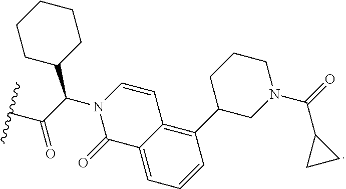 [SMILES: *C(=O)[C@@H](C1CCCCC1)n1ccc2c(C3CCCN(C(=O)C4CC4)C3)cccc2c1=O]

In some embodiments, DBM is

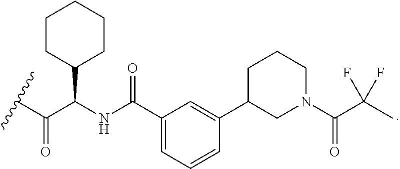 [SMILES: *C(=O)[C@H](NC(=O)c1cccc(C2CCCN(C(=O)C(C)(F)F)C2)c1)C1CCCCC1]

In some embodiments, DBM is

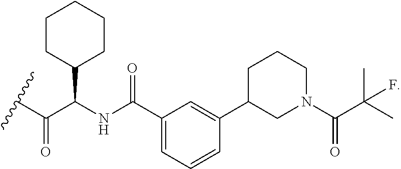 [SMILES: *C(=O)[C@H](NC(=O)c1cccc(C2CCCN(C(=O)C(C)(C)F)C2)c1)C1CCCCC1]

In some embodiments, DBM is

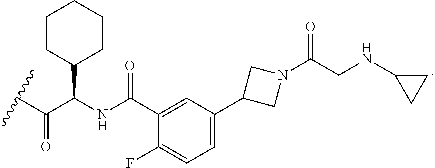 [SMILES: *C(=O)[C@H](NC(=O)c1cc(C2CN(C(=O)CNC3CC3)C2)ccc1F)C1CCCCC1]

In some embodiments, DBM is

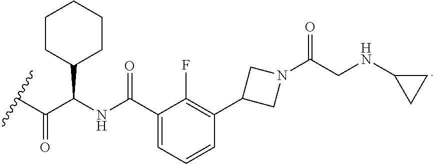 [SMILES: *C(=O)[C@H](NC(=O)c1cccc(C2CN(C(=O)CNC3CC3)C2)c1F)C1CCCCC1]

In some embodiments, DBM is

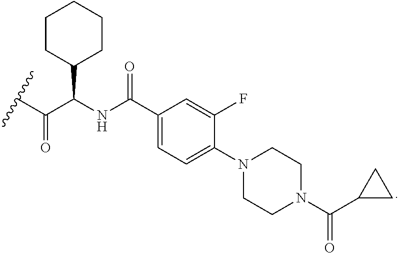 [SMILES: *C(=O)[C@H](NC(=O)c1ccc(N2CCN(C(=O)C3CC3)CC2)c(F)c1)C1CCCCC1]

In some embodiments, DBM is

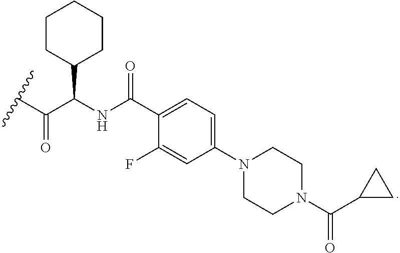 [SMILES: *C(=O)[C@H](NC(=O)c1ccc(N2CCN(C(=O)C3CC3)CC2)cc1F)C1CCCCC1]

In some embodiments, DBM is

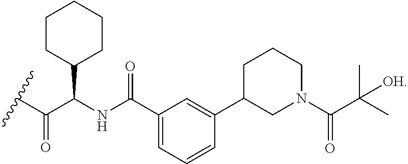 [SMILES: *C(=O)[C@H](NC(=O)c1cccc(C2CCCN(C(=O)C(C)(C)O)C2)c1)C1CCCCC1]

In some embodiments, DBM is

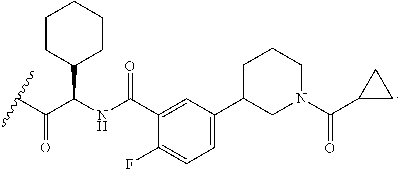 [SMILES: *C(=O)[C@H](NC(=O)c1cc(C2CCCN(C(=O)C3CC3)C2)ccc1F)C1CCCCC1]

In some embodiments, DBM is

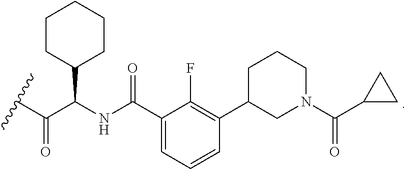 [SMILES: *C(=O)[C@H](NC(=O)c1cccc(C2CCCN(C(=O)C3CC3)C2)c1F)C1CCCCC1]

In some embodiments, DBM is

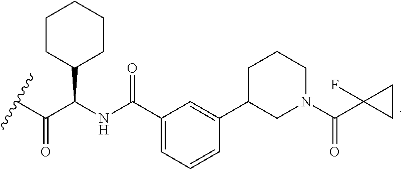 [SMILES: *C(=O)[C@H](NC(=O)c1cccc(C2CCCN(C(=O)C3(F)CC3)C2)c1)C1CCCCC1]

In some embodiments, DBM is

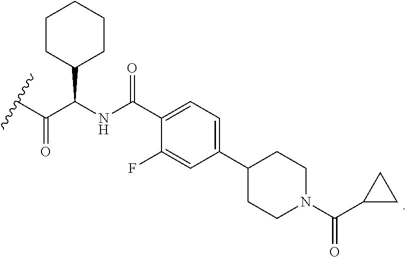 [SMILES: *C(=O)[C@H](NC(=O)c1ccc(C2CCN(C(=O)C3CC3)CC2)cc1F)C1CCCCC1]

In some embodiments, DBM is

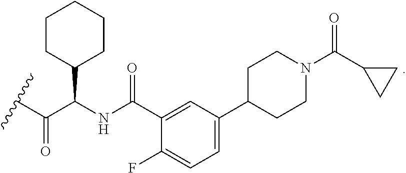 [SMILES: *C(=O)[C@H](NC(=O)c1cc(C2CCN(C(=O)C3CC3)CC2)ccc1F)C1CCCCC1]

In some embodiments, DBM is

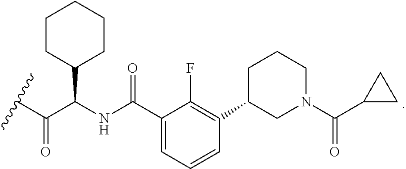 [SMILES: *C(=O)[C@H](NC(=O)c1cccc([C@@H]2CCCN(C(=O)C3CC3)C2)c1F)C1CCCCC1]

In some embodiments, DBM is

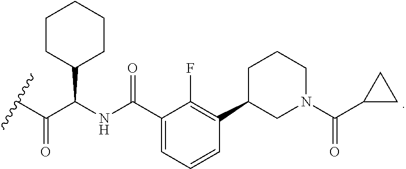 [SMILES: *C(=O)[C@H](NC(=O)c1cccc([C@H]2CCCN(C(=O)C3CC3)C2)c1F)C1CCCCC1]

In some embodiments, DBM is

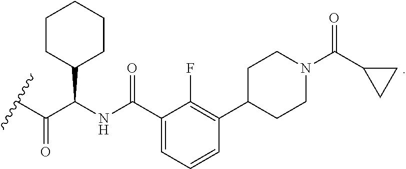 [SMILES: *C(=O)[C@H](NC(=O)c1cccc(C2CCN(C(=O)C3CC3)CC2)c1F)C1CCCCC1]

In some embodiments, DBM is

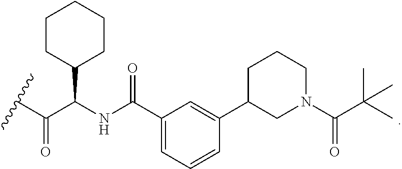 [SMILES: *C(=O)[C@H](NC(=O)c1cccc(C2CCCN(C(=O)C(C)(C)C)C2)c1)C1CCCCC1]

In some embodiments, DBM is

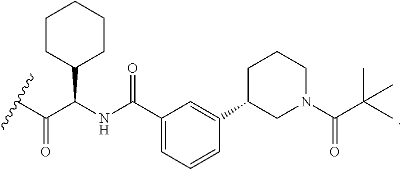 [SMILES: *C(=O)[C@H](NC(=O)c1cccc([C@@H]2CCCN(C(=O)C(C)(C)C)C2)c1)C1CCCCC1]

In some embodiments, DBM is

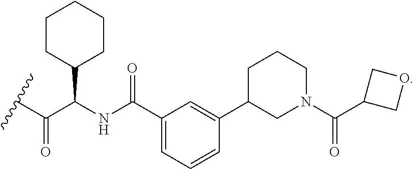 [SMILES: *C(=O)[C@H](NC(=O)c1cccc(C2CCCN(C(=O)C3COC3)C2)c1)C1CCCCC1]

In some embodiments, DBM is

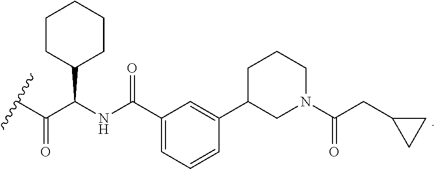 [SMILES: *C(=O)[C@H](NC(=O)c1cccc(C2CCCN(C(=O)CC3CC3)C2)c1)C1CCCCC1]

In some embodiments, DBM is

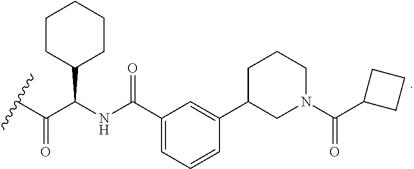 [SMILES: *C(=O)[C@H](NC(=O)c1cccc(C2CCCN(C(=O)C3CCC3)C2)c1)C1CCCCC1]

In some embodiments, DBM is

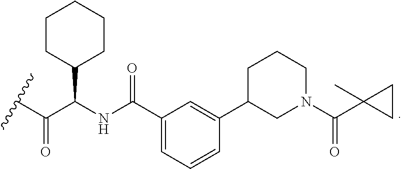 [SMILES: *C(=O)[C@H](NC(=O)c1cccc(C2CCCN(C(=O)C3(C)CC3)C2)c1)C1CCCCC1]

In some embodiments, DBM is

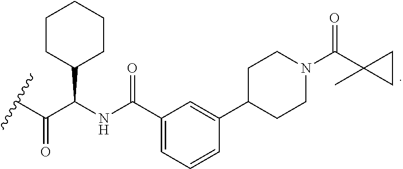 [SMILES: *C(=O)[C@H](NC(=O)c1cccc(C2CCN(C(=O)C3(C)CC3)CC2)c1)C1CCCCC1]

In some embodiments, DBM is

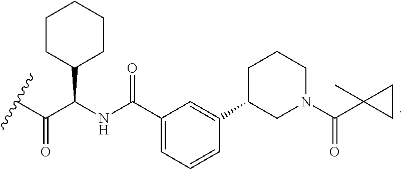 [SMILES: *C(=O)[C@H](NC(=O)c1cccc([C@@H]2CCCN(C(=O)C3(C)CC3)C2)c1)C1CCCCC1]

In some embodiments, DBM is

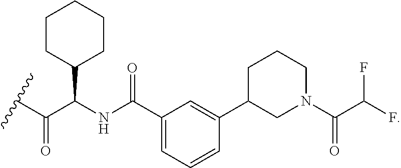 [SMILES: *C(=O)[C@H](NC(=O)c1cccc(C2CCCN(C(=O)C(F)F)C2)c1)C1CCCCC1]

In some embodiments, DBM is

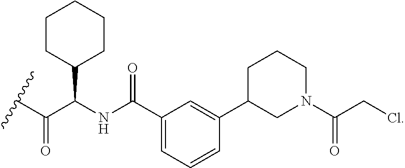 [SMILES: *C(=O)[C@H](NC(=O)c1cccc(C2CCCN(C(=O)CCl)C2)c1)C1CCCCC1]

In some embodiments, DBM is

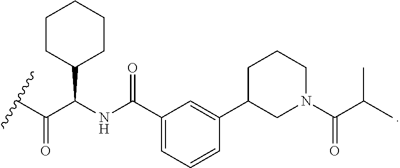 [SMILES: *C(=O)[C@H](NC(=O)c1cccc(C2CCCN(C(=O)C(C)C)C2)c1)C1CCCCC1]

In some embodiments, DBM is

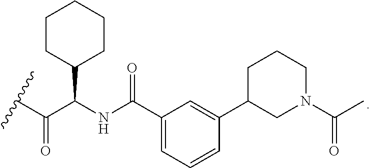 [SMILES: *C(=O)[C@H](NC(=O)c1cccc(C2CCCN(C(C)=O)C2)c1)C1CCCCC1]

In some embodiments, DBM is

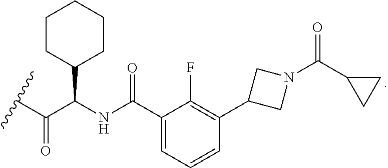 [SMILES: *C(=O)[C@H](NC(=O)c1cccc(C2CN(C(=O)C3CC3)C2)c1F)C1CCCCC1]

In some embodiments, DBM is

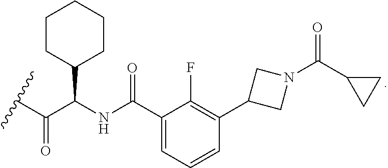 [SMILES: *C(=O)[C@H](NC(=O)c1cccc(C2CN(C(=O)C3CC3)C2)c1F)C1CCCCC1]

In some embodiments, DBM is

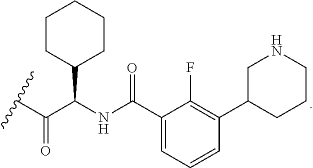 [SMILES: *C(=O)[C@H](NC(=O)c1cccc(C2CCCNC2)c1F)C1CCCCC1]

In some embodiments, DBM is

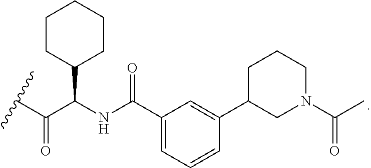 [SMILES: *C(=O)[C@H](NC(=O)c1cccc(C2CCCN(C(C)=O)C2)c1)C1CCCCC1]

In some embodiments, DBM is

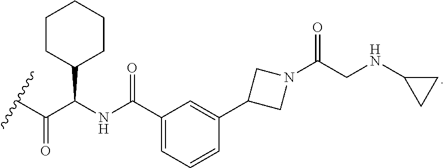 [SMILES: *C(=O)[C@H](NC(=O)c1cccc(C2CN(C(=O)CNC3CC3)C2)c1)C1CCCCC1]

In some embodiments, DBM is

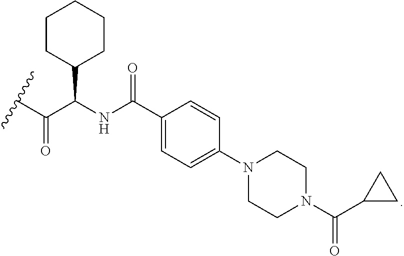 [SMILES: *C(=O)[C@H](NC(=O)c1ccc(N2CCN(C(=O)C3CC3)CC2)cc1)C1CCCCC1]

In some embodiments, DBM is

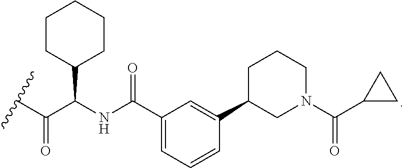 [SMILES: *C(=O)[C@H](NC(=O)c1cccc([C@H]2CCCN(C(=O)C3CC3)C2)c1)C1CCCCC1]

In some embodiments, DBM is

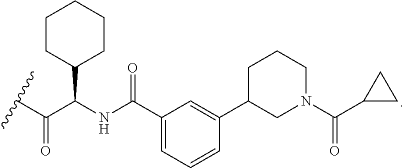 [SMILES: *C(=O)[C@H](NC(=O)c1cccc(C2CCCN(C(=O)C3CC3)C2)c1)C1CCCCC1]

In some embodiments, DBM is

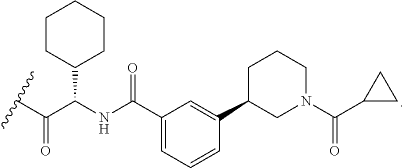 [SMILES: *C(=O)[C@@H](NC(=O)c1cccc([C@H]2CCCN(C(=O)C3CC3)C2)c1)C1CCCCC1]

In some embodiments, DBM is

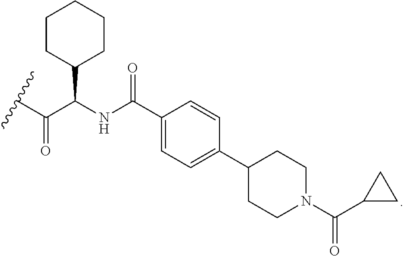 [SMILES: *C(=O)[C@H](NC(=O)c1ccc(C2CCN(C(=O)C3CC3)CC2)cc1)C1CCCCC1]

In some embodiments, DBM is

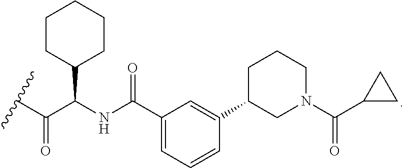 [SMILES: *C(=O)[C@H](NC(=O)c1cccc([C@@H]2CCCN(C(=O)C3CC3)C2)c1)C1CCCCC1]

In some embodiments, DBM is

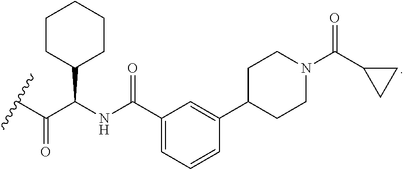 [SMILES: *C(=O)[C@H](NC(=O)c1cccc(C2CCN(C(=O)C3CC3)CC2)c1)C1CCCCC1]

In some embodiments, DBM is

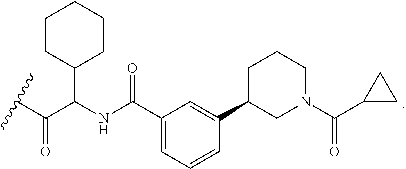 [SMILES: *C(=O)C(NC(=O)c1cccc([C@H]2CCCN(C(=O)C3CC3)C2)c1)C1CCCCC1]

In some embodiments, DBM is

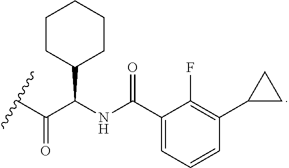 [SMILES: *C(=O)[C@H](NC(=O)c1cccc(C2CC2)c1F)C1CCCCC1]

In some embodiments, DBM is

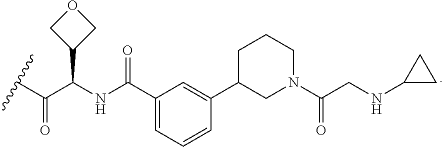 [SMILES: *C(=O)[C@H](NC(=O)c1cccc(C2CCCN(C(=O)CNC3CC3)C2)c1)C1COC1]

In some embodiments, DBM is

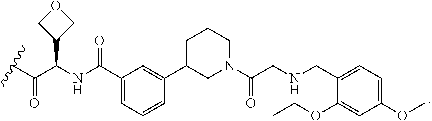 [SMILES: *C(=O)[C@H](NC(=O)c1cccc(C2CCCN(C(=O)CNCc3ccc(OC)cc3OCC)C2)c1)C1COC1]

In some embodiments, DBM is

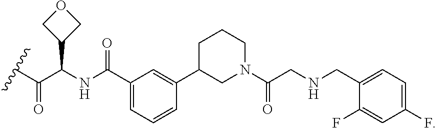 [SMILES: *C(=O)[C@H](NC(=O)c1cccc(C2CCCN(C(=O)CNCc3ccc(F)cc3F)C2)c1)C1COC1]

In some embodiments, DBM is

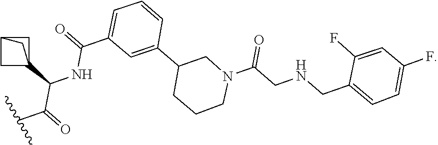 [SMILES: *C(=O)[C@H](NC(=O)c1cccc(C2CCCN(C(=O)CNCc3ccc(F)cc3F)C2)c1)C12CC(C1)C2]

In some embodiments, DBM is

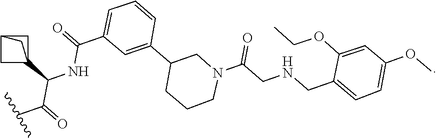 [SMILES: *C(=O)[C@H](NC(=O)c1cccc(C2CCCN(C(=O)CNCc3ccc(OC)cc3OCC)C2)c1)C12CC(C1)C2]

In some embodiments, DBM is

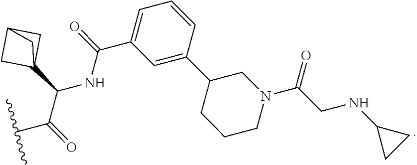 [SMILES: *C(=O)[C@H](NC(=O)c1cccc(C2CCCN(C(=O)CNC3CC3)C2)c1)C12CC(C1)C2]

In some embodiments, DBM is

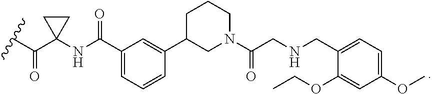 [SMILES: *C(=O)C1(NC(=O)c2cccc(C3CCCN(C(=O)CNCc4ccc(OC)cc4OCC)C3)c2)CC1]

In some embodiments, DBM is

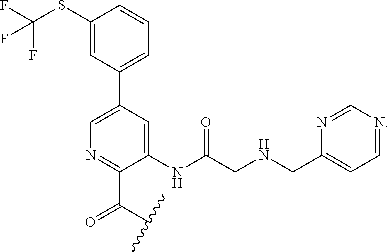 [SMILES: *C(=O)c1ncc(-c2cccc(SC(F)(F)F)c2)cc1NC(=O)CNCc1ccncn1]

In some embodiments, DBM is

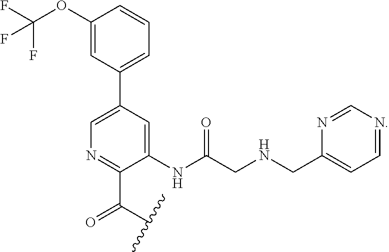 [SMILES: *C(=O)c1ncc(-c2cccc(OC(F)(F)F)c2)cc1NC(=O)CNCc1ccncn1]

In some embodiments, DBM is

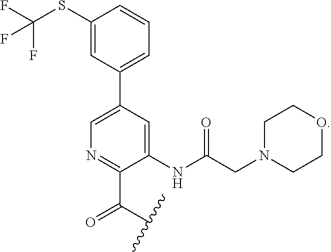 [SMILES: *C(=O)c1ncc(-c2cccc(SC(F)(F)F)c2)cc1NC(=O)CN1CCOCC1]

In some embodiments, DBM is

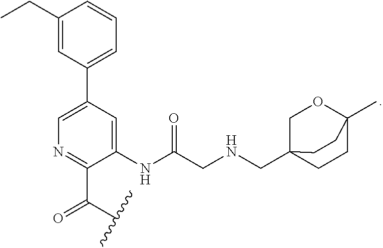 [SMILES: *C(=O)c1ncc(-c2cccc(CC)c2)cc1NC(=O)CNCC12CCC(C)(CC1)OC2]

In some embodiments, DBM is

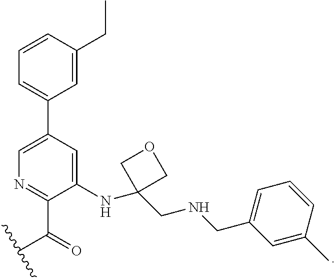 [SMILES: *C(=O)c1ncc(-c2cccc(CC)c2)cc1NC1(CNCc2cccc(C)c2)COC1]

In some embodiments, DBM is

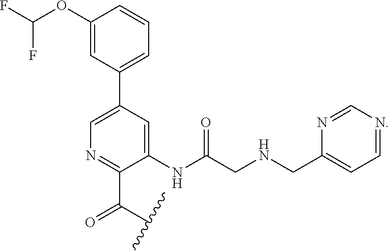 [SMILES: *C(=O)c1ncc(-c2cccc(OC(F)F)c2)cc1NC(=O)CNCc1ccncn1]

In some embodiments, DBM is

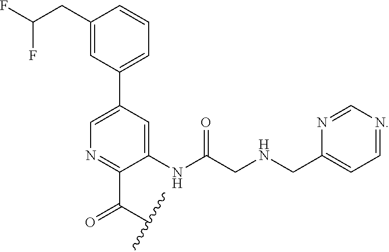 [SMILES: *C(=O)c1ncc(-c2cccc(CC(F)F)c2)cc1NC(=O)CNCc1ccncn1]

In some embodiments, DBM is

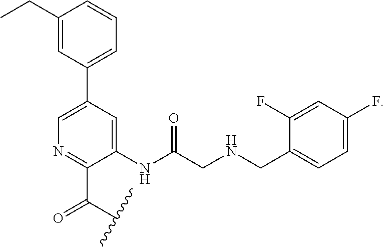 [SMILES: *C(=O)c1ncc(-c2cccc(CC)c2)cc1NC(=O)CNCc1ccc(F)cc1F]

In some embodiments, DBM is

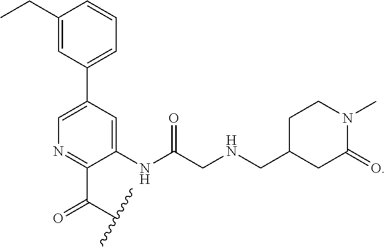 [SMILES: *C(=O)c1ncc(-c2cccc(CC)c2)cc1NC(=O)CNCC1CCN(C)C(=O)C1]

In some embodiments, DBM is

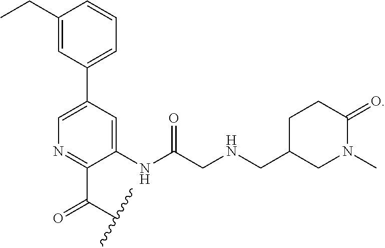 [SMILES: *C(=O)c1ncc(-c2cccc(CC)c2)cc1NC(=O)CNCC1CCC(=O)N(C)C1]

In some embodiments, DBM is

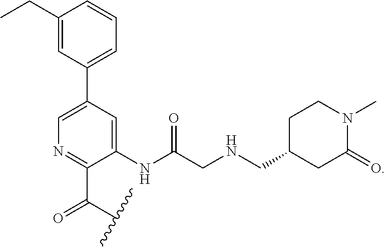 [SMILES: *C(=O)c1ncc(-c2cccc(CC)c2)cc1NC(=O)CNC[C@@H]1CCN(C)C(=O)C1]

In some embodiments, DBM is

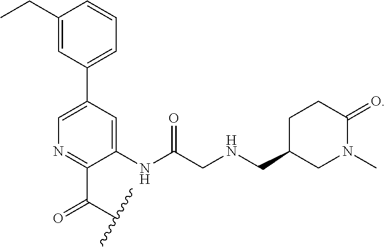 [SMILES: *C(=O)c1ncc(-c2cccc(CC)c2)cc1NC(=O)CNC[C@H]1CCC(=O)N(C)C1]

In some embodiments, DBM is

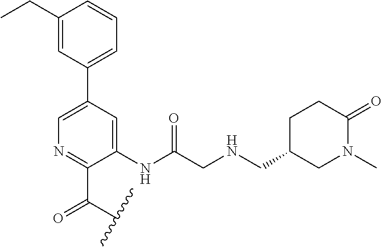 [SMILES: *C(=O)c1ncc(-c2cccc(CC)c2)cc1NC(=O)CNC[C@@H]1CCC(=O)N(C)C1]

In some embodiments, DBM is

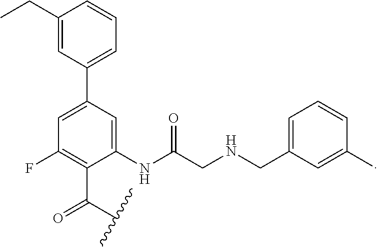

In some embodiments, DBM is

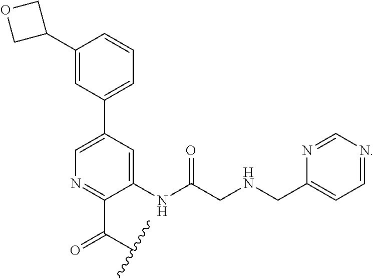 [SMILES: *C(=O)c1ncc(-c2cccc(C3COC3)c2)cc1NC(=O)CNCc1ccncn1]

In some embodiments, DBM is

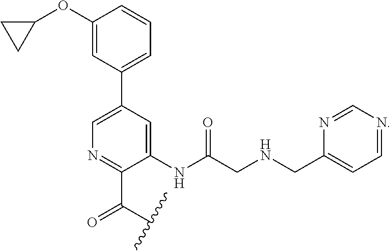 [SMILES: *C(=O)c1ncc(-c2cccc(OC3CC3)c2)cc1NC(=O)CNCc1ccncn1]

In some embodiments, DBM is

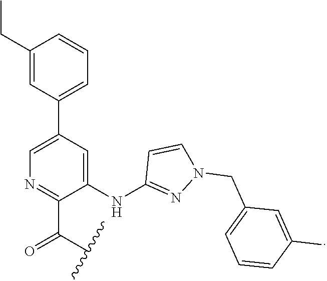

In some embodiments, DBM is

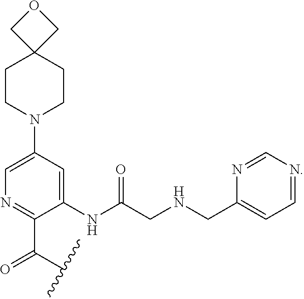 [SMILES: *C(=O)c1ncc(N2CCC3(CC2)COC3)cc1NC(=O)CNCc1ccncn1]

In some embodiments, DBM is

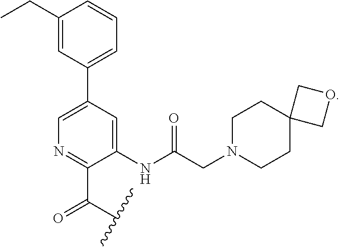 [SMILES: *C(=O)c1ncc(-c2cccc(CC)c2)cc1NC(=O)CN1CCC2(CC1)COC2]

In some embodiments, DBM is

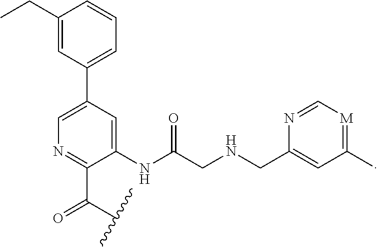

In some embodiments, DBM is

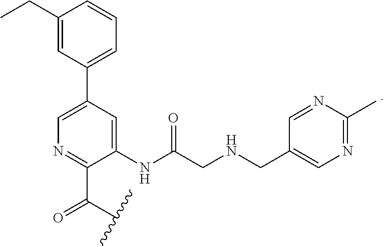 [SMILES: *C(=O)c1ncc(-c2cccc(CC)c2)cc1NC(=O)CNCc1cnc(C)nc1]

In some embodiments, DBM is

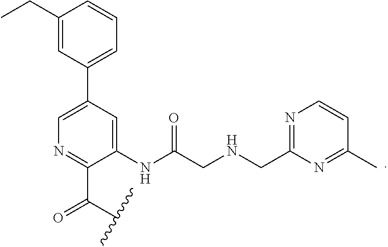 [SMILES: *C(=O)c1ncc(-c2cccc(CC)c2)cc1NC(=O)CNCc1nccc(C)n1]

In some embodiments, DBM is

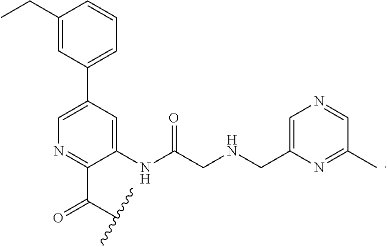 [SMILES: *C(=O)c1ncc(-c2cccc(CC)c2)cc1NC(=O)CNCc1cncc(C)n1]

In some embodiments, DBM is

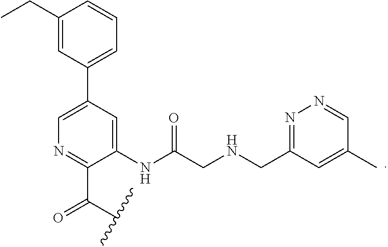 [SMILES: *C(=O)c1ncc(-c2cccc(CC)c2)cc1NC(=O)CNCc1cc(C)cnn1]

In some embodiments, DBM is

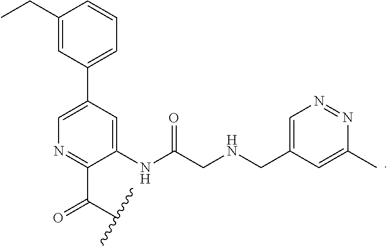 [SMILES: *C(=O)c1ncc(-c2cccc(CC)c2)cc1NC(=O)CNCc1cnnc(C)c1]

In some embodiments, DBM is

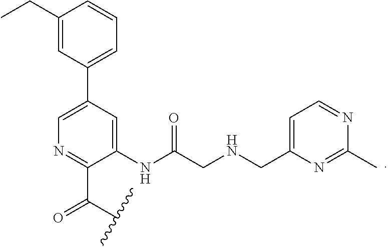 [SMILES: *C(=O)c1ncc(-c2cccc(CC)c2)cc1NC(=O)CNCc1ccnc(C)n1]

In some embodiments, DBM is

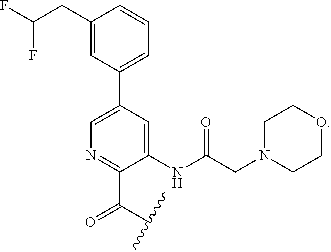 [SMILES: *C(=O)c1ncc(-c2cccc(CC(F)F)c2)cc1NC(=O)CN1CCOCC1]

In some embodiments, DBM is

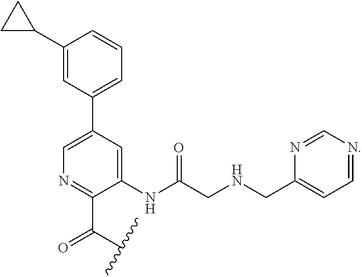 [SMILES: *C(=O)c1ncc(-c2cccc(C3CC3)c2)cc1NC(=O)CNCc1ccncn1]

In some embodiments, DBM is

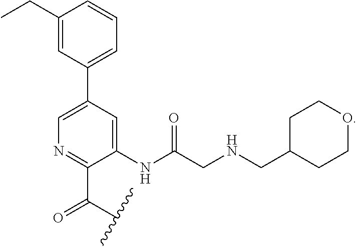 [SMILES: *C(=O)c1ncc(-c2cccc(CC)c2)cc1NC(=O)CNCC1CCOCC1]

In some embodiments, DBM is

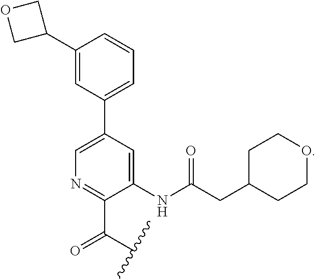 [SMILES: *C(=O)c1ncc(-c2cccc(C3COC3)c2)cc1NC(=O)CC1CCOCC1]

In some embodiments, DBM is

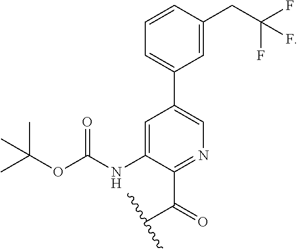 [SMILES: *C(=O)c1ncc(-c2cccc(CC(F)(F)F)c2)cc1NC(=O)OC(C)(C)C]

In some embodiments, DBM is

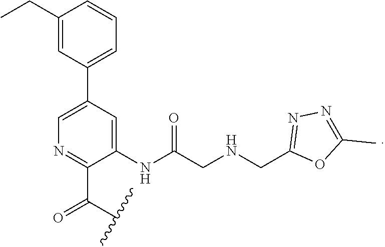 [SMILES: *C(=O)c1ncc(-c2cccc(CC)c2)cc1NC(=O)CNCc1nnc(C)o1]

In some embodiments, DBM is

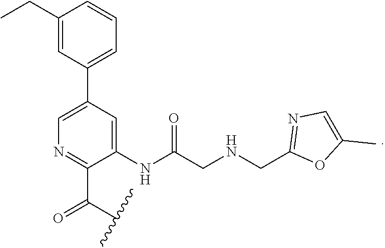 [SMILES: *C(=O)c1ncc(-c2cccc(CC)c2)cc1NC(=O)CNCc1ncc(C)o1]

In some embodiments, DBM is

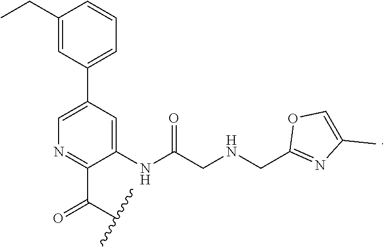 [SMILES: *C(=O)c1ncc(-c2cccc(CC)c2)cc1NC(=O)CNCc1nc(C)co1]

In some embodiments, DBM is

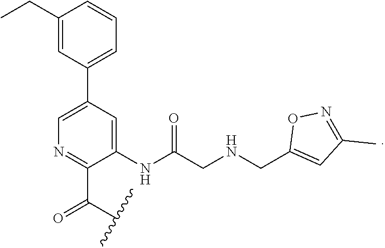 [SMILES: *C(=O)c1ncc(-c2cccc(CC)c2)cc1NC(=O)CNCc1cc(C)no1]

In some embodiments, DBM is

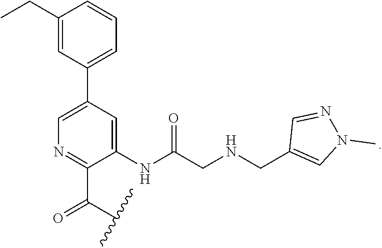 [SMILES: *C(=O)c1ncc(-c2cccc(CC)c2)cc1NC(=O)CNCc1cnn(C)c1]

In some embodiments, DBM is

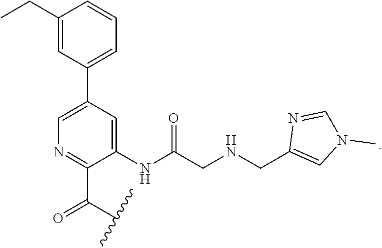 [SMILES: *C(=O)c1ncc(-c2cccc(CC)c2)cc1NC(=O)CNCc1cn(C)cn1]

In some embodiments, DBM is

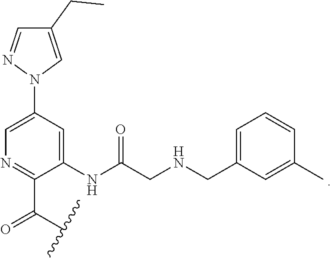 [SMILES: *C(=O)c1ncc(-n2cc(CC)cn2)cc1NC(=O)CNCc1cccc(C)c1]

In some embodiments, DBM is

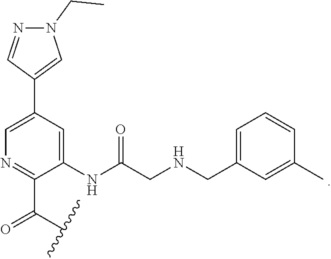 [SMILES: *C(=O)c1ncc(-c2cnn(CC)c2)cc1NC(=O)CNCc1cccc(C)c1]

In some embodiments, DBM is

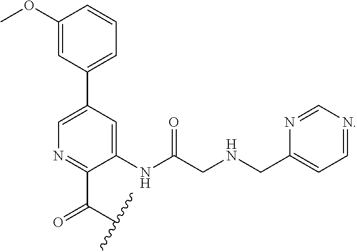 [SMILES: *C(=O)c1ncc(-c2cccc(OC)c2)cc1NC(=O)CNCc1ccncn1]

In some embodiments, DBM is

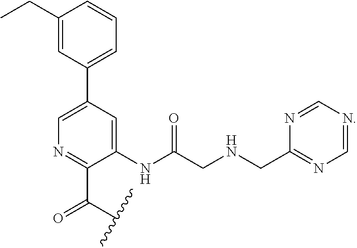 [SMILES: *C(=O)c1ncc(-c2cccc(CC)c2)cc1NC(=O)CNCc1ncncn1]

In some embodiments, DBM is

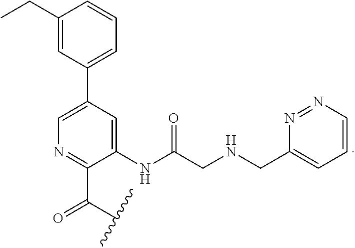 [SMILES: *C(=O)c1ncc(-c2cccc(CC)c2)cc1NC(=O)CNCc1cccnn1]

In some embodiments, DBM is

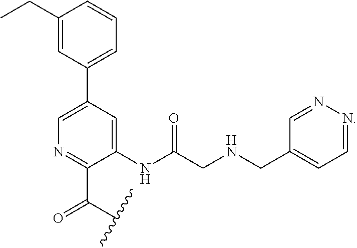 [SMILES: *C(=O)c1ncc(-c2cccc(CC)c2)cc1NC(=O)CNCc1ccnnc1]

In some embodiments, DBM is

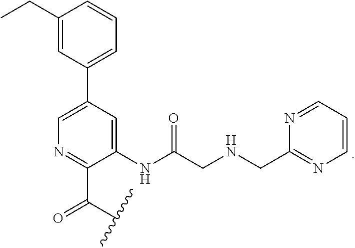 [SMILES: *C(=O)c1ncc(-c2cccc(CC)c2)cc1NC(=O)CNCc1ncccn1]

In some embodiments, DBM is

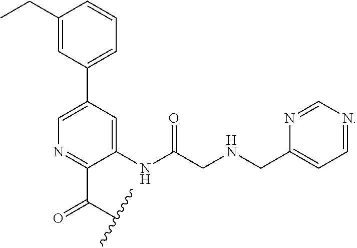 [SMILES: *C(=O)c1ncc(-c2cccc(CC)c2)cc1NC(=O)CNCc1ccncn1]

In some embodiments, DBM is

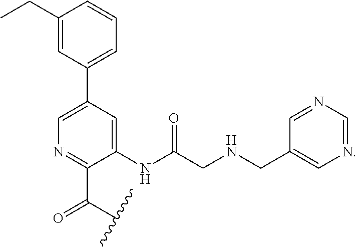 [SMILES: *C(=O)c1ncc(-c2cccc(CC)c2)cc1NC(=O)CNCc1cncnc1]

In some embodiments, DBM is

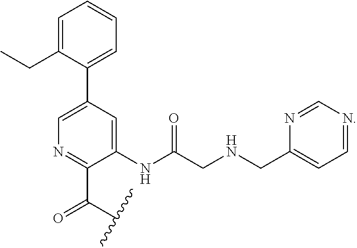 [SMILES: *C(=O)c1ncc(-c2ccccc2CC)cc1NC(=O)CNCc1ccncn1]

In some embodiments, DBM is

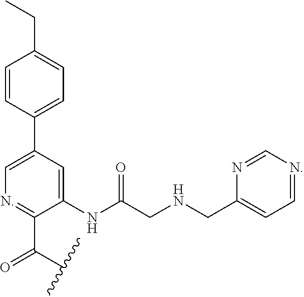 [SMILES: *C(=O)c1ncc(-c2ccc(CC)cc2)cc1NC(=O)CNCc1ccncn1]

In some embodiments, DBM is

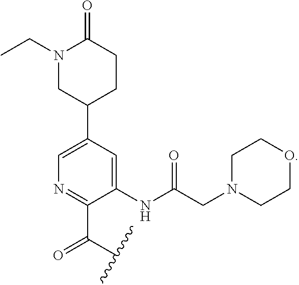 [SMILES: *C(=O)c1ncc(C2CCC(=O)N(CC)C2)cc1NC(=O)CN1CCOCC1]

In some embodiments, DBM is

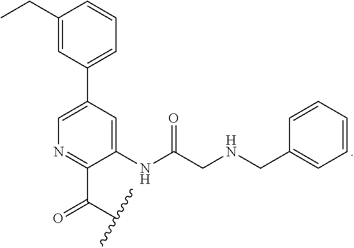 [SMILES: *C(=O)c1ncc(-c2cccc(CC)c2)cc1NC(=O)CNCc1ccccc1]

In some embodiments, DBM is

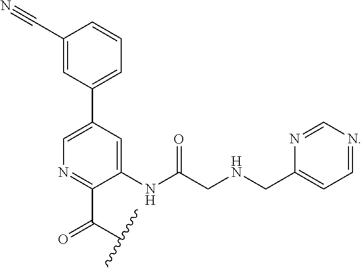 [SMILES: *C(=O)c1ncc(-c2cccc(C#N)c2)cc1NC(=O)CNCc1ccncn1]

In some embodiments, DBM is

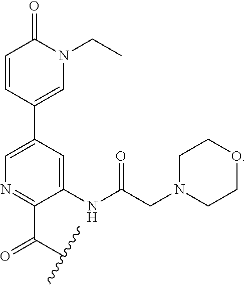 [SMILES: *C(=O)c1ncc(-c2ccc(=O)n(CC)c2)cc1NC(=O)CN1CCOCC1]

In some embodiments, DBM is

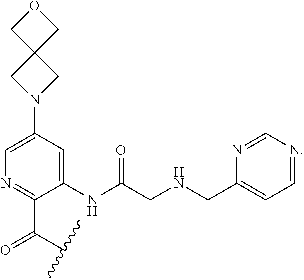 [SMILES: *C(=O)c1ncc(N2CC3(COC3)C2)cc1NC(=O)CNCc1ccncn1]

In some embodiments, DBM is

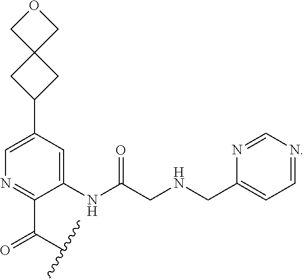 [SMILES: *C(=O)c1ncc(C2CC3(COC3)C2)cc1NC(=O)CNCc1ccncn1]

In some embodiments, DBM is

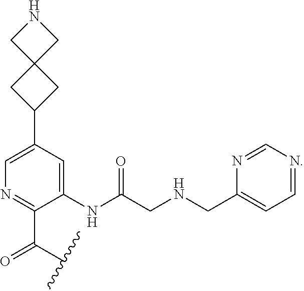 [SMILES: *C(=O)c1ncc(C2CC3(CNC3)C2)cc1NC(=O)CNCc1ccncn1]

In some embodiments, DBM is

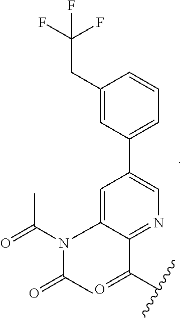 [SMILES: *C(=O)c1ncc(-c2cccc(CC(F)(F)F)c2)cc1N(C(C)=O)C(C)=O]

In some embodiments, DBM is

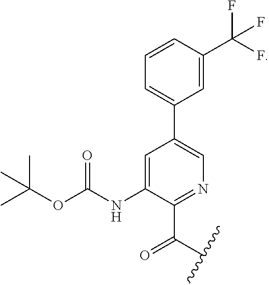 [SMILES: *C(=O)c1ncc(-c2cccc(C(F)(F)F)c2)cc1NC(=O)OC(C)(C)C]

In some embodiments, DBM is

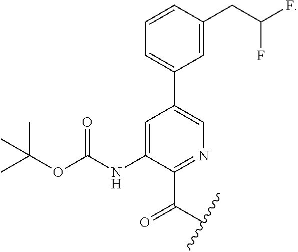 [SMILES: *C(=O)c1ncc(-c2cccc(CC(F)F)c2)cc1NC(=O)OC(C)(C)C]

In some embodiments, DBM is

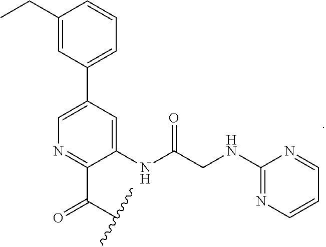 [SMILES: *C(=O)c1ncc(-c2cccc(CC)c2)cc1NC(=O)CNc1ncccn1]

In some embodiments, DBM is

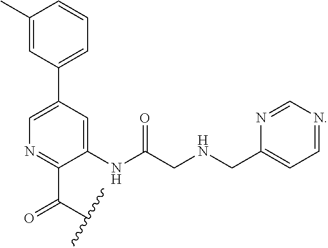 [SMILES: *C(=O)c1ncc(-c2cccc(C)c2)cc1NC(=O)CNCc1ccncn1]

In some embodiments, DBM is

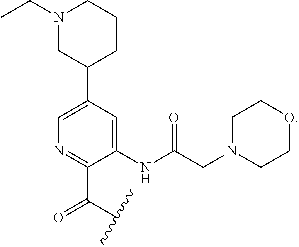 [SMILES: *C(=O)c1ncc(C2CCCN(CC)C2)cc1NC(=O)CN1CCOCC1]

In some embodiments, DBM is DBM is

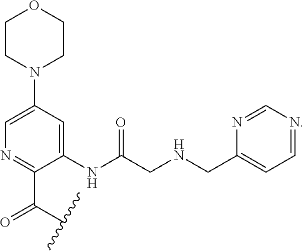 [SMILES: *C(=O)c1ncc(N2CCOCC2)cc1NC(=O)CNCc1ccncn1]

In some embodiments, DBM is

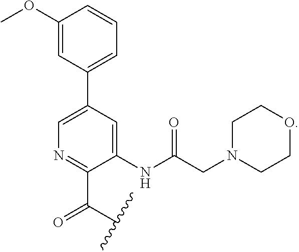 [SMILES: *C(=O)c1ncc(-c2cccc(OC)c2)cc1NC(=O)CN1CCOCC1]

In some embodiments, DBM is

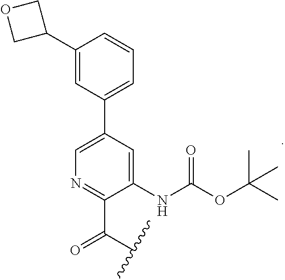 [SMILES: *C(=O)c1ncc(-c2cccc(C3COC3)c2)cc1NC(=O)OC(C)(C)C]

In some embodiments, DBM is

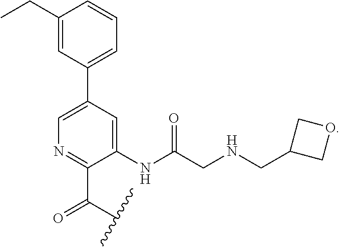 [SMILES: *C(=O)c1ncc(-c2cccc(CC)c2)cc1NC(=O)CNCC1COC1]

In some embodiments, DBM is

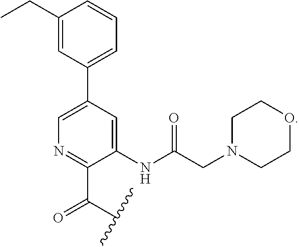 [SMILES: *C(=O)c1ncc(-c2cccc(CC)c2)cc1NC(=O)CN1CCOCC1]

In some embodiments, DBM is

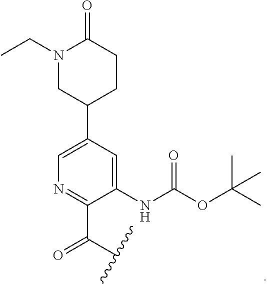 [SMILES: *C(=O)c1ncc(C2CCC(=O)N(CC)C2)cc1NC(=O)OC(C)(C)C]

In some embodiments, DBM is

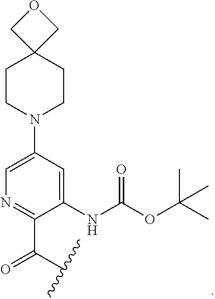 [SMILES: *C(=O)c1ncc(N2CCC3(CC2)COC3)cc1NC(=O)OC(C)(C)C]

In some embodiments, DBM is

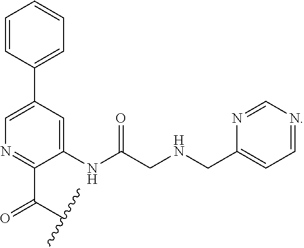 [SMILES: *C(=O)c1ncc(-c2ccccc2)cc1NC(=O)CNCc1ccncn1]

In some embodiments, DBM is

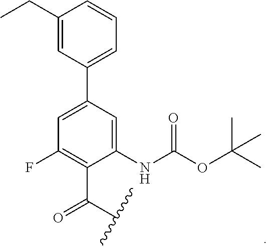 [SMILES: *C(=O)c1c(F)cc(-c2cccc(CC)c2)cc1NC(=O)OC(C)(C)C]

In some embodiments, DBM is

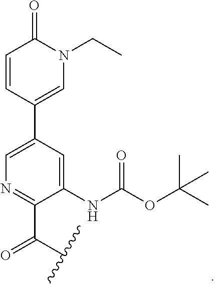 [SMILES: *C(=O)c1ncc(-c2ccc(=O)n(CC)c2)cc1NC(=O)OC(C)(C)C]

In some embodiments, DBM is

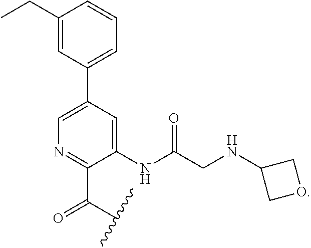 [SMILES: *C(=O)c1ncc(-c2cccc(CC)c2)cc1NC(=O)CNC1COC1]

In some embodiments, DBM is

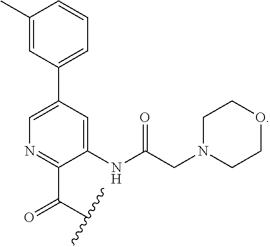 [SMILES: *C(=O)c1ncc(-c2cccc(C)c2)cc1NC(=O)CN1CCOCC1]

In some embodiments, DBM is

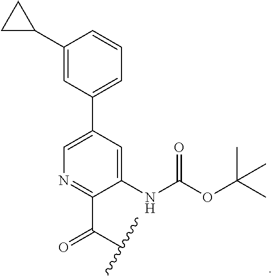 [SMILES: *C(=O)c1ncc(-c2cccc(C3CC3)c2)cc1NC(=O)OC(C)(C)C]

In some embodiments, DBM is

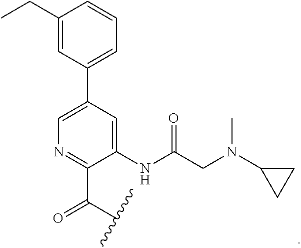 [SMILES: *C(=O)c1ncc(-c2cccc(CC)c2)cc1NC(=O)CN(C)C1CC1]

In some embodiments, DBM is

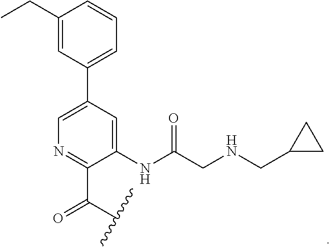 [SMILES: *C(=O)c1ncc(-c2cccc(CC)c2)cc1NC(=O)CNCC1CC1]

In some embodiments, DBM is

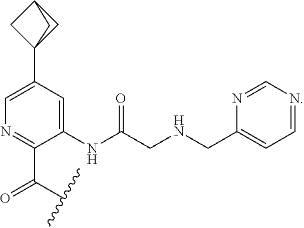 [SMILES: *C(=O)c1ncc(C23CC(C2)C3)cc1NC(=O)CNCc1ccncn1]

In some embodiments, DBM is

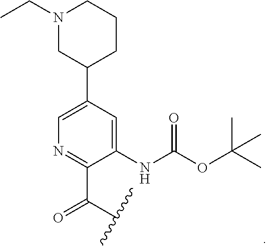 [SMILES: *C(=O)c1ncc(C2CCCN(CC)C2)cc1NC(=O)OC(C)(C)C]

In some embodiments, DBM is

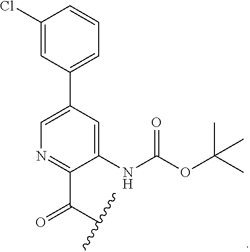 [SMILES: *C(=O)c1ncc(-c2cccc(Cl)c2)cc1NC(=O)OC(C)(C)C]

In some embodiments, DBM is

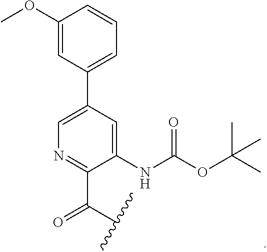 [SMILES: *C(=O)c1ncc(-c2cccc(OC)c2)cc1NC(=O)OC(C)(C)C]

In some embodiments, DBM is

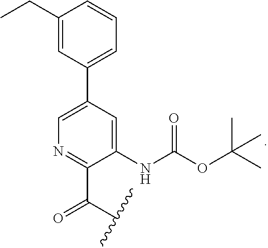 [SMILES: *C(=O)c1ncc(-c2cccc(CC)c2)cc1NC(=O)OC(C)(C)C]

In some embodiments, DBM is

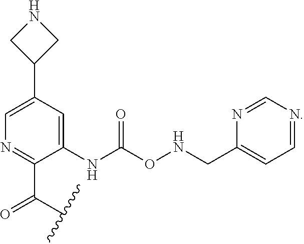 [SMILES: *C(=O)c1ncc(C2CNC2)cc1NC(=O)ONCc1ccncn1]

In some embodiments, DBM is

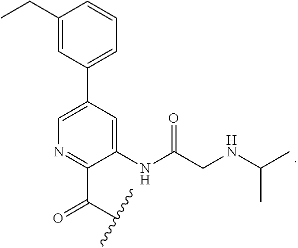 [SMILES: *C(=O)c1ncc(-c2cccc(CC)c2)cc1NC(=O)CNC(C)C]

In some embodiments, DBM is

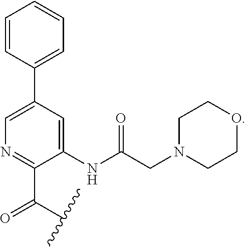 [SMILES: *C(=O)c1ncc(-c2ccccc2)cc1NC(=O)CN1CCOCC1]

In some embodiments, DBM is

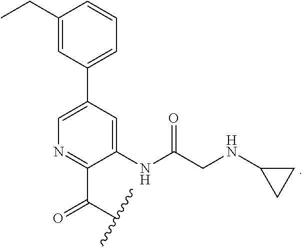 [SMILES: *C(=O)c1ncc(-c2cccc(CC)c2)cc1NC(=O)CNC1CC1]

In some embodiments, DBM is is

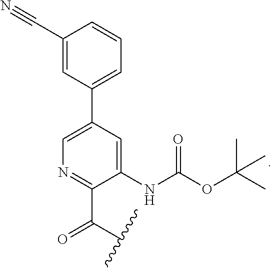 [SMILES: *C(=O)c1ncc(-c2cccc(C#N)c2)cc1NC(=O)OC(C)(C)C]

In some embodiments, DBM is

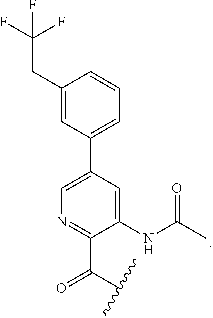 [SMILES: *C(=O)c1ncc(-c2cccc(CC(F)(F)F)c2)cc1NC(C)=O]

In some embodiments, DBM is

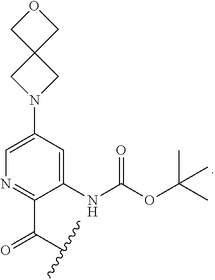 [SMILES: *C(=O)c1ncc(N2CC3(COC3)C2)cc1NC(=O)OC(C)(C)C]

In some embodiments, DBM is

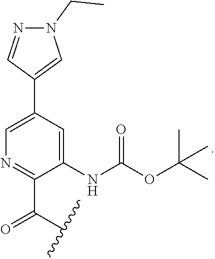 [SMILES: *C(=O)c1ncc(-c2cnn(CC)c2)cc1NC(=O)OC(C)(C)C]

In some embodiments, DBM is

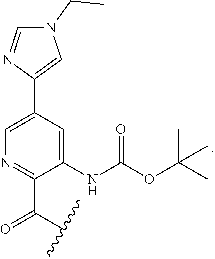 [SMILES: *C(=O)c1ncc(-c2cn(CC)cn2)cc1NC(=O)OC(C)(C)C]

In some embodiments, DBM is

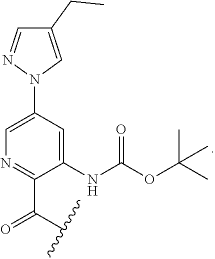 [SMILES: *C(=O)c1ncc(-n2cc(CC)cn2)cc1NC(=O)OC(C)(C)C]

In some embodiments, DBM is

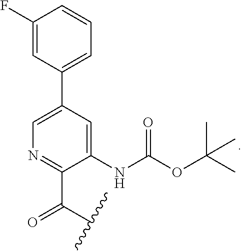 [SMILES: *C(=O)c1ncc(-c2cccc(F)c2)cc1NC(=O)OC(C)(C)C]

In some embodiments, DBM is

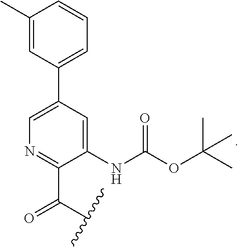 [SMILES: *C(=O)c1ncc(-c2cccc(C)c2)cc1NC(=O)OC(C)(C)C]

In some embodiments, DBM is

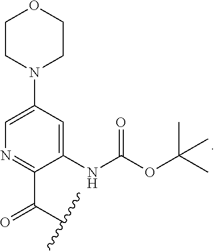 [SMILES: *C(=O)c1ncc(N2CCOCC2)cc1NC(=O)OC(C)(C)C]

In some embodiments, DBM is

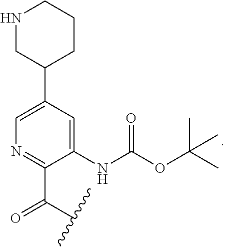 [SMILES: *C(=O)c1ncc(C2CCCNC2)cc1NC(=O)OC(C)(C)C]

In some embodiments, DBM is

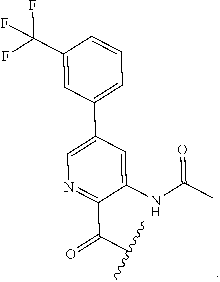 [SMILES: *C(=O)c1ncc(-c2cccc(C(F)(F)F)c2)cc1NC(C)=O]

In some embodiments, DBM is

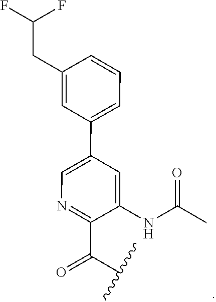 [SMILES: *C(=O)c1ncc(-c2cccc(CC(F)F)c2)cc1NC(C)=O]

In some embodiments, DBM is

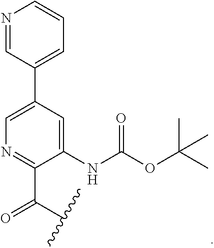 [SMILES: *C(=O)c1ncc(-c2cccnc2)cc1NC(=O)OC(C)(C)C]

In some embodiments, DBM is

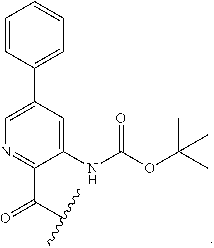 [SMILES: *C(=O)c1ncc(-c2ccccc2)cc1NC(=O)OC(C)(C)C]

In some embodiments, DBM is

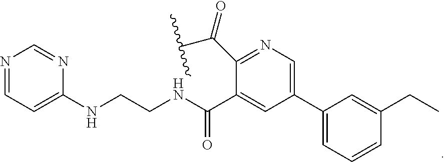 [SMILES: *C(=O)c1ncc(-c2cccc(CC)c2)cc1C(=O)NCCNc1ccncn1]

In some embodiments, DBM is

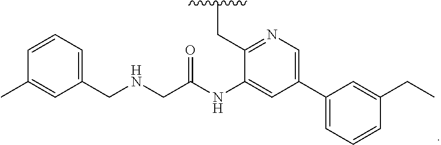 [SMILES: *Cc1ncc(-c2cccc(CC)c2)cc1NC(=O)CNCc1cccc(C)c1]

In some embodiments, DBM is

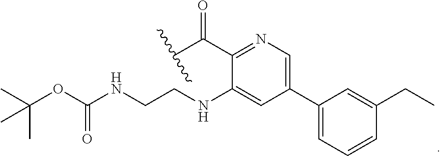 [SMILES: *C(=O)c1ncc(-c2cccc(CC)c2)cc1NCCNC(=O)OC(C)(C)C]

In some embodiments, DBM is

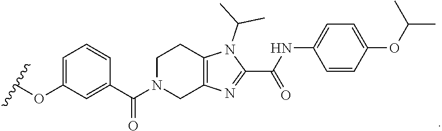 [SMILES: *Oc1cccc(C(=O)N2CCc3c(nc(C(=O)Nc4ccc(OC(C)C)cc4)n3C(C)C)C2)c1]

In some embodiments, DBM is

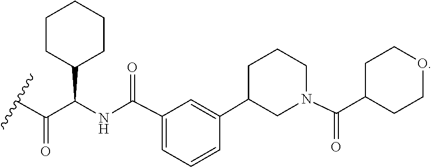 [SMILES: *C(=O)[C@H](NC(=O)c1cccc(C2CCCN(C(=O)C3CCOCC3)C2)c1)C1CCCCC1]

In some embodiments, DBM is

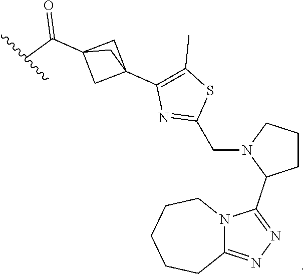 [SMILES: *C(=O)C12CC(c3nc(CN4CCCC4c4nnc5n4CCCCC5)sc3C)(C1)C2]

In some embodiments, DBM is

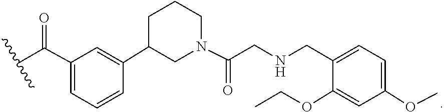 [SMILES: *C(=O)c1cccc(C2CCCN(C(=O)CNCc3ccc(OC)cc3OCC)C2)c1]

In some embodiments, DBM is

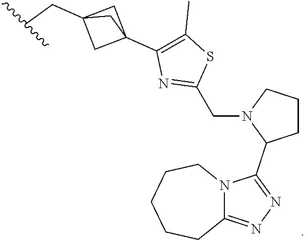 [SMILES: *CC12CC(c3nc(CN4CCCC4c4nnc5n4CCCCC5)sc3C)(C1)C2]

In some embodiments, DBM is

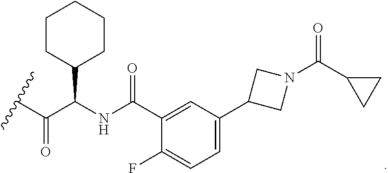 [SMILES: *C(=O)[C@H](NC(=O)c1cc(C2CN(C(=O)C3CC3)C2)ccc1F)C1CCCCC1]

In some embodiments, DBM is

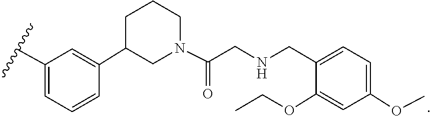 [SMILES: *c1cccc(C2CCCN(C(=O)CNCc3ccc(OC)cc3OCC)C2)c1]

In some embodiments, DBM is

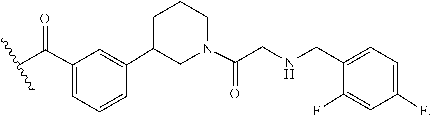 [SMILES: *C(=O)c1cccc(C2CCCN(C(=O)CNCc3ccc(F)cc3F)C2)c1]

In some embodiments, DBM is

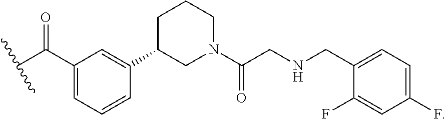 [SMILES: *C(=O)c1cccc([C@@H]2CCCN(C(=O)CNCc3ccc(F)cc3F)C2)c1]

In some embodiments, DBM is

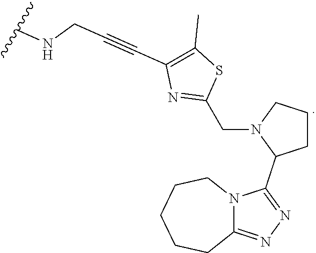 [SMILES: *NCC#Cc1nc(CN2CCCC2c2nnc3n2CCCCC3)sc1C]

In some embodiments, DBM is

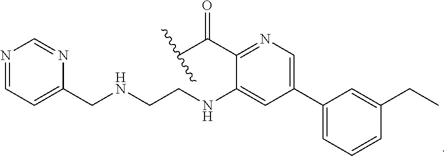 [SMILES: *C(=O)c1ncc(-c2cccc(CC)c2)cc1NCCNCc1ccncn1]

In some embodiments, DBM is

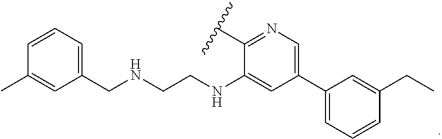 [SMILES: *c1ncc(-c2cccc(CC)c2)cc1NCCNCc1cccc(C)c1]

In some embodiments, DBM is

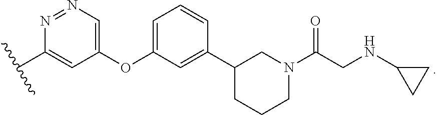 [SMILES: *c1cc(Oc2cccc(C3CCCN(C(=O)CNC4CC4)C3)c2)cnn1]

In some embodiments, DBM is

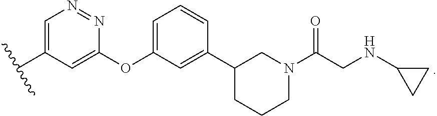 [SMILES: *c1cnnc(Oc2cccc(C3CCCN(C(=O)CNC4CC4)C3)c2)c1]

In some embodiments, DBM is

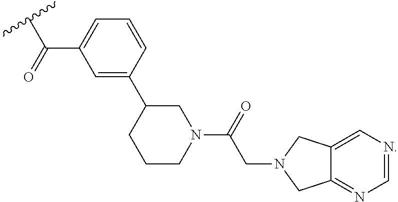 [SMILES: *C(=O)c1cccc(C2CCCN(C(=O)CN3Cc4cncnc4C3)C2)c1]

In some embodiments, DBM is

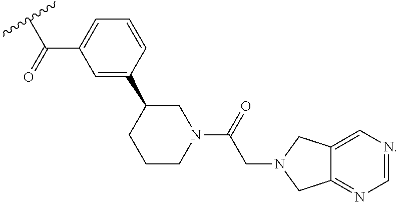 [SMILES: *C(=O)c1cccc([C@@H]2CCCN(C(=O)CN3Cc4cncnc4C3)C2)c1]

In some embodiments, DBM is

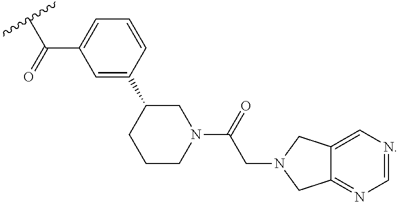 [SMILES: *C(=O)c1cccc([C@H]2CCCN(C(=O)CN3Cc4cncnc4C3)C2)c1]

In some embodiments, DBM is

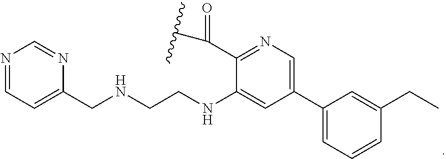 [SMILES: *C(=O)c1ncc(-c2cccc(CC)c2)cc1NCCNCc1ccncn1]

In some embodiments, DBM is

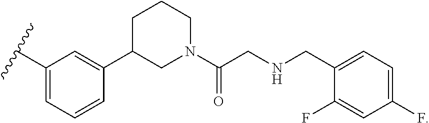 [SMILES: *c1cccc(C2CCCN(C(=O)CNCc3ccc(F)cc3F)C2)c1]

In some embodiments, DBM is

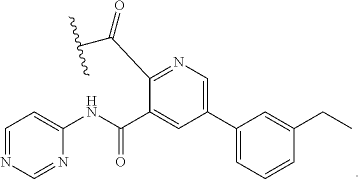 [SMILES: *C(=O)c1ncc(-c2cccc(CC)c2)cc1C(=O)Nc1ccncn1]

In some embodiments, DBM is

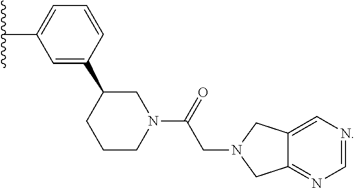 [SMILES: *c1cccc([C@@H]2CCCN(C(=O)CN3Cc4cncnc4C3)C2)c1]

In some embodiments, DBM is

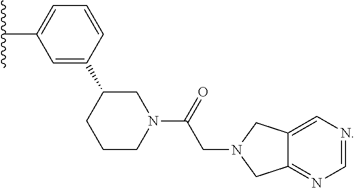 [SMILES: *c1cccc([C@H]2CCCN(C(=O)CN3Cc4cncnc4C3)C2)c1]

In some embodiments, DBM is

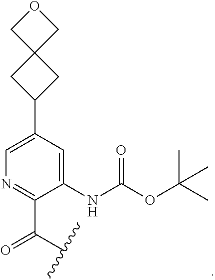 [SMILES: *C(=O)c1ncc(C2CC3(COC3)C2)cc1NC(=O)OC(C)(C)C]

In some embodiments, DBM is

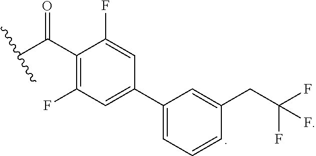 [SMILES: *C(=O)c1c(F)cc(-c2cccc(CC(F)(F)F)c2)cc1F]

In some embodiments, DBM is

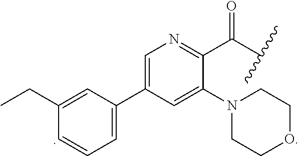 [SMILES: *C(=O)c1ncc(-c2cccc(CC)c2)cc1N1CCOCC1]

In some embodiments, DBM is

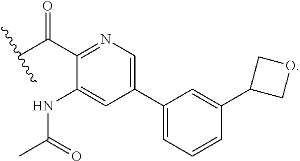 [SMILES: *C(=O)c1ncc(-c2cccc(C3COC3)c2)cc1NC(C)=O]

In some embodiments, DBM is

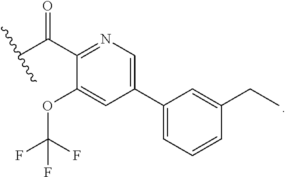 [SMILES: *C(=O)c1ncc(-c2cccc(CC)c2)cc1OC(F)(F)F]

In some embodiments, DBM is

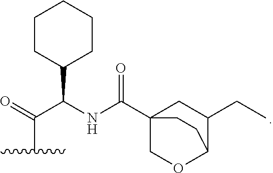 [SMILES: *C(=O)[C@H](NC(=O)C12CCC(OC1)C(CC)C2)C1CCCCC1]

In some embodiments, DBM is

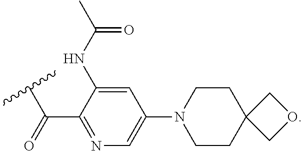 [SMILES: *C(=O)c1ncc(N2CCC3(CC2)COC3)cc1NC(C)=O]

In some embodiments, DBM is

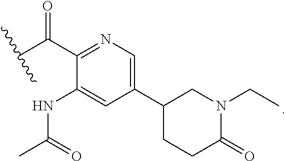 [SMILES: *C(=O)c1ncc(C2CCC(=O)N(CC)C2)cc1NC(C)=O]

In some embodiments, DBM is

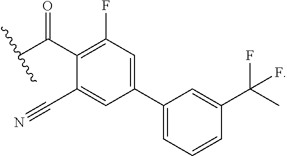 [SMILES: *C(=O)c1c(F)cc(-c2cccc(C(C)(F)F)c2)cc1C#N]

In some embodiments, DBM is

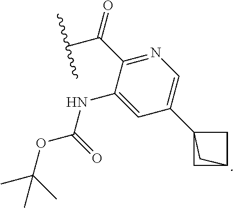 [SMILES: *C(=O)c1ncc(C23CC(C2)C3)cc1NC(=O)OC(C)(C)C]

In some embodiments, DBM is

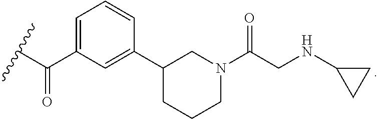 [SMILES: *C(=O)c1cccc(C2CCCN(C(=O)CNC3CC3)C2)c1]

In some embodiments, DBM is

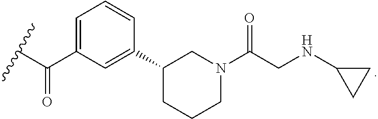 [SMILES: *C(=O)c1cccc([C@H]2CCCN(C(=O)CNC3CC3)C2)c1]

In some embodiments, DBM is

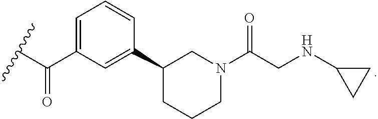 [SMILES: *C(=O)c1cccc([C@@H]2CCCN(C(=O)CNC3CC3)C2)c1]

In some embodiments, DBM is

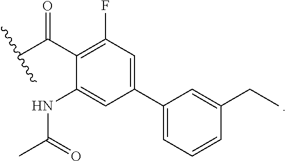 [SMILES: *C(=O)c1c(F)cc(-c2cccc(CC)c2)cc1NC(C)=O]

In some embodiments, DBM is

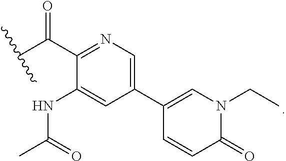 [SMILES: *C(=O)c1ncc(-c2ccc(=O)n(CC)c2)cc1NC(C)=O]

In some embodiments, DBM is

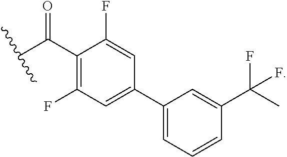 [SMILES: *C(=O)c1c(F)cc(-c2cccc(C(C)(F)F)c2)cc1F]

In some embodiments, DBM is

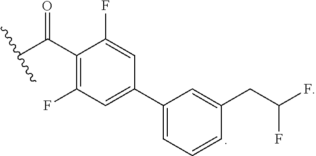 [SMILES: *C(=O)c1c(F)cc(-c2cccc(CC(F)F)c2)cc1F]

In some embodiments, DBM is

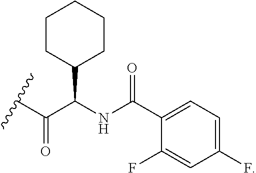 [SMILES: *C(=O)[C@H](NC(=O)c1ccc(F)cc1F)C1CCCCC1]

In some embodiments, DBM is

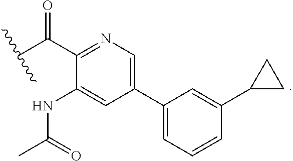 [SMILES: *C(=O)c1ncc(-c2cccc(C3CC3)c2)cc1NC(C)=O]

In some embodiments, DBM is

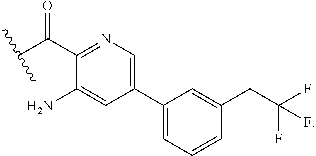 [SMILES: *C(=O)c1ncc(-c2cccc(CC(F)(F)F)c2)cc1N]

In some embodiments, DBM is

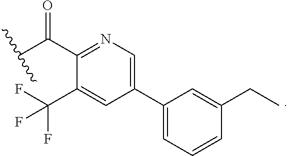 [SMILES: *C(=O)c1ncc(-c2cccc(CC)c2)cc1C(F)(F)F]

In some embodiments, DBM is

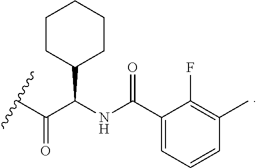 [SMILES: *C(=O)[C@H](NC(=O)c1cccc(C)c1F)C1CCCCC1]

In some embodiments, DBM is

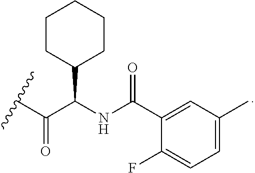 [SMILES: *C(=O)[C@H](NC(=O)c1cc(C)ccc1F)C1CCCCC1]

In some embodiments, DBM is

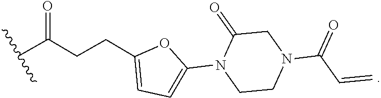 [SMILES: *C(=O)CCc1ccc(N2CCN(C(=O)C=C)CC2=O)o1]

In some embodiments, DBM is

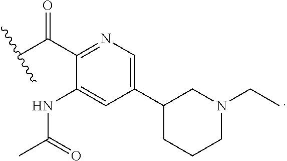 [SMILES: *C(=O)c1ncc(C2CCCN(CC)C2)cc1NC(C)=O]

In some embodiments, DBM is

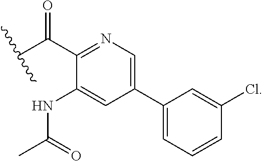 [SMILES: *C(=O)c1ncc(-c2cccc(Cl)c2)cc1NC(C)=O]

In some embodiments, DBM is

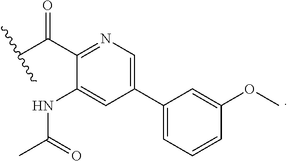 [SMILES: *C(=O)c1ncc(-c2cccc(OC)c2)cc1NC(C)=O]

In some embodiments, DBM is

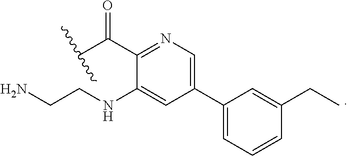 [SMILES: *C(=O)c1ncc(-c2cccc(CC)c2)cc1NCCN]

In some embodiments, DBM is

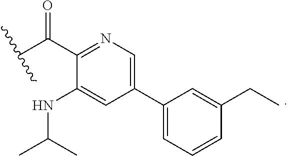 [SMILES: *C(=O)c1ncc(-c2cccc(CC)c2)cc1NC(C)C]

In some embodiments, DBM is

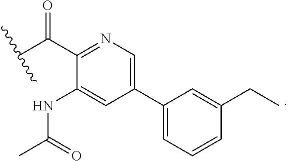 [SMILES: *C(=O)c1ncc(-c2cccc(CC)c2)cc1NC(C)=O]

In some embodiments, DBM is

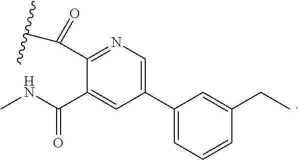 [SMILES: *C(=O)c1ncc(-c2cccc(CC)c2)cc1C(=O)NC]

In some embodiments, DBM is

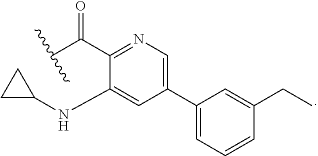 [SMILES: *C(=O)c1ncc(-c2cccc(CC)c2)cc1NC1CC1]

In some embodiments, DBM is

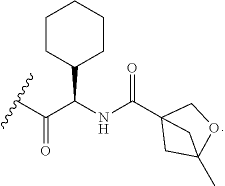 [SMILES: *C(=O)[C@H](NC(=O)C12COC(C)(C1)C2)C1CCCCC1]

In some embodiments, DBM is

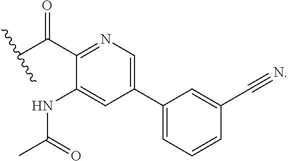 [SMILES: *C(=O)c1ncc(-c2cccc(C#N)c2)cc1NC(C)=O]

In some embodiments, DBM is

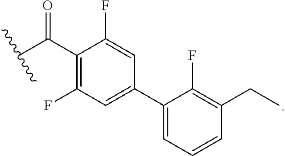 [SMILES: *C(=O)c1c(F)cc(-c2cccc(CC)c2F)cc1F]

In some embodiments, DBM is

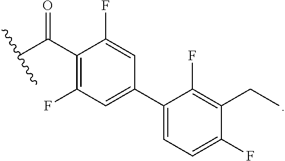 [SMILES: *C(=O)c1c(F)cc(-c2ccc(F)c(CC)c2F)cc1F]

In some embodiments, DBM is

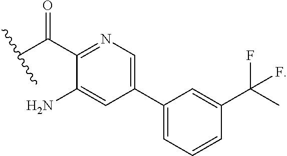 [SMILES: *C(=O)c1ncc(-c2cccc(C(C)(F)F)c2)cc1N]

In some embodiments, DBM is

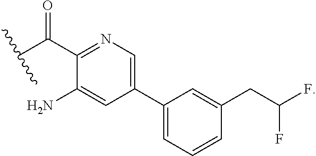 [SMILES: *C(=O)c1ncc(-c2cccc(CC(F)F)c2)cc1N]

In some embodiments, DBM is

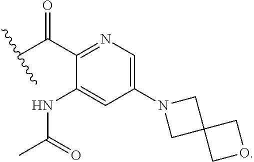 [SMILES: *C(=O)c1ncc(N2CC3(COC3)C2)cc1NC(C)=O]

In some embodiments, DBM is

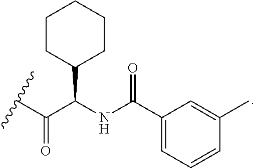 [SMILES: *C(=O)[C@H](NC(=O)c1cccc(C)c1)C1CCCCC1]

In some embodiments, DBM is

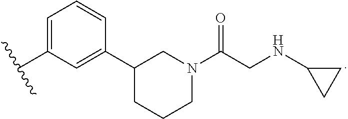 [SMILES: *c1cccc(C2CCCN(C(=O)CNC3CC3)C2)c1]

In some embodiments, DBM is

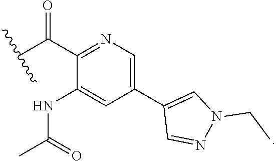 [SMILES: *C(=O)c1ncc(-c2cnn(CC)c2)cc1NC(C)=O]

In some embodiments, DBM is

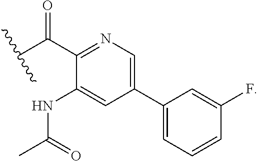 [SMILES: *C(=O)c1ncc(-c2cccc(F)c2)cc1NC(C)=O]

In some embodiments, DBM is

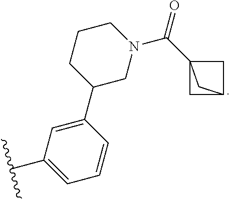 [SMILES: *c1cccc(C2CCCN(C(=O)C34CC(C3)C4)C2)c1]

In some embodiments, DBM is

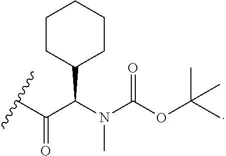 [SMILES: *C(=O)[C@@H](C1CCCCC1)N(C)C(=O)OC(C)(C)C]

In some embodiments, DBM is

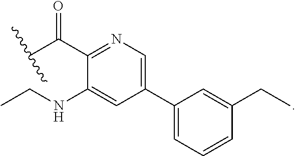 [SMILES: *C(=O)c1ncc(-c2cccc(CC)c2)cc1NCC]

In some embodiments, DBM is

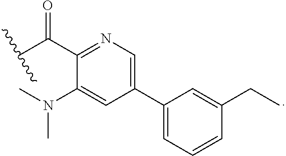 [SMILES: *C(=O)c1ncc(-c2cccc(CC)c2)cc1N(C)C]

In some embodiments, DBM is

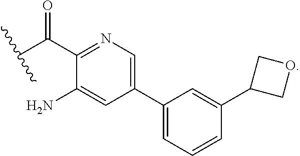 [SMILES: *C(=O)c1ncc(-c2cccc(C3COC3)c2)cc1N]

In some embodiments, DBM is

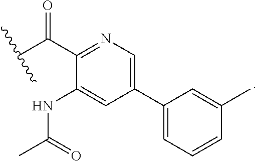 [SMILES: *C(=O)c1ncc(-c2cccc(C)c2)cc1NC(C)=O]

In some embodiments, DBM is

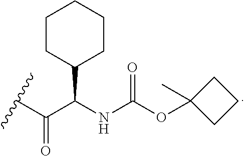 [SMILES: *C(=O)[C@H](NC(=O)OC1(C)CCC1)C1CCCCC1]

In some embodiments, DBM is

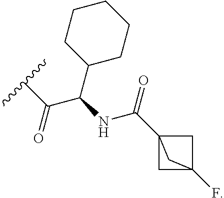 [SMILES: *C(=O)[C@H](NC(=O)C12CC(F)(C1)C2)C1CCCCC1]

In some embodiments, DBM is

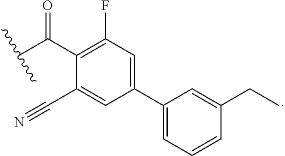 [SMILES: *C(=O)c1c(F)cc(-c2cccc(CC)c2)cc1C#N]

In some embodiments, DBM is

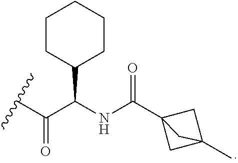 [SMILES: *C(=O)[C@H](NC(=O)C12CC(C)(C1)C2)C1CCCCC1]

In some embodiments, DBM is

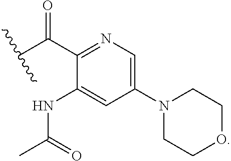 [SMILES: *C(=O)c1ncc(N2CCOCC2)cc1NC(C)=O]

In some embodiments, DBM is

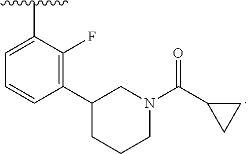 [SMILES: *c1cccc(C2CCCN(C(=O)C3CC3)C2)c1F]

In some embodiments, DBM is

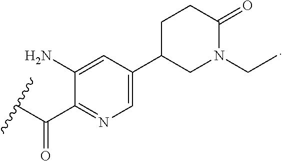 [SMILES: *C(=O)c1ncc(C2CCC(=O)N(CC)C2)cc1N]

In some embodiments, DBM is

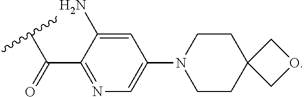 [SMILES: *C(=O)c1ncc(N2CCC3(CC2)COC3)cc1N]

In some embodiments, DBM is

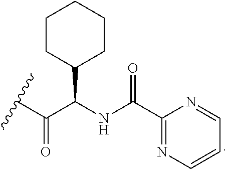 [SMILES: *C(=O)[C@H](NC(=O)c1ncccn1)C1CCCCC1]

In some embodiments, DBM is

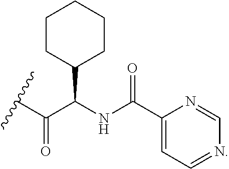 [SMILES: *C(=O)[C@H](NC(=O)c1ccncn1)C1CCCCC1]

In some embodiments, DBM is

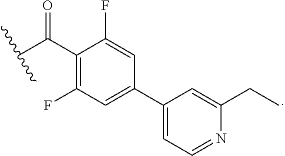 [SMILES: *C(=O)c1c(F)cc(-c2ccnc(CC)c2)cc1F]

In some embodiments, DBM is

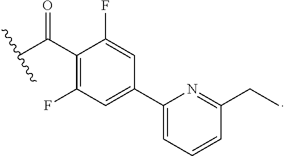 [SMILES: *C(=O)c1c(F)cc(-c2cccc(CC)n2)cc1F]

In some embodiments, DBM is

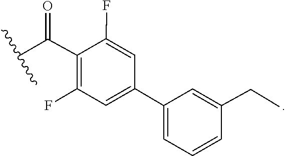 [SMILES: *C(=O)c1c(F)cc(-c2cccc(CC)c2)cc1F]

In some embodiments, DBM is

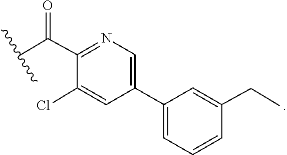 [SMILES: *C(=O)c1ncc(-c2cccc(CC)c2)cc1Cl]

In some embodiments, DBM is

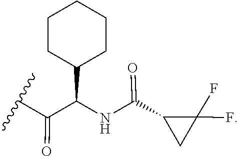 [SMILES: *C(=O)[C@H](NC(=O)[C@H]1CC1(F)F)C1CCCCC1]

In some embodiments, DBM is

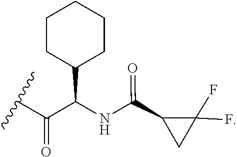 [SMILES: *C(=O)[C@H](NC(=O)[C@@H]1CC1(F)F)C1CCCCC1]

In some embodiments, DBM is

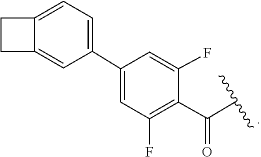 [SMILES: *C(=O)c1c(F)cc(-c2ccc3c(c2)CC3)cc1F]

In some embodiments, DBM is

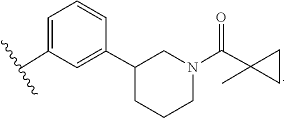 [SMILES: *c1cccc(C2CCCN(C(=O)C3(C)CC3)C2)c1]

In some embodiments, DBM is

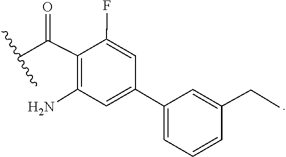 [SMILES: *C(=O)c1c(N)cc(-c2cccc(CC)c2)cc1F]

In some embodiments, DBM is

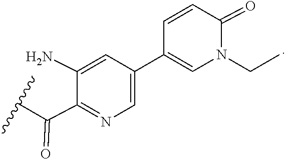 [SMILES: *C(=O)c1ncc(-c2ccc(=O)n(CC)c2)cc1N]

In some embodiments, DBM is

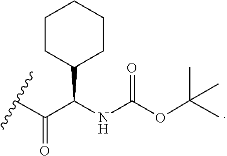 [SMILES: *C(=O)[C@H](NC(=O)OC(C)(C)C)C1CCCCC1]

In some embodiments, DBM is

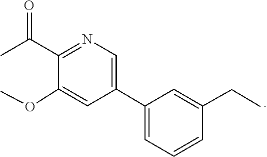 [SMILES: CCc1cccc(-c2cnc(C(C)=O)c(OC)c2)c1]

In some embodiments, DBM is

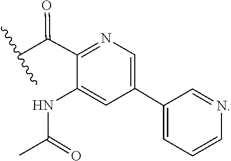 [SMILES: *C(=O)c1ncc(-c2cccnc2)cc1NC(C)=O]

In some embodiments, DBM is

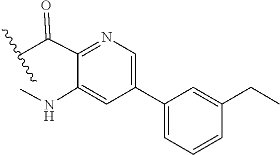 [SMILES: *C(=O)c1ncc(-c2cccc(CC)c2)cc1NC]

In some embodiments, DBM is

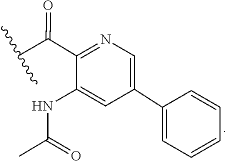 [SMILES: *C(=O)c1ncc(-c2ccccc2)cc1NC(C)=O]

In some embodiments, DBM is

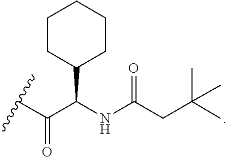 [SMILES: *C(=O)[C@H](NC(=O)CC(C)(C)C)C1CCCCC1]

In some embodiments, DBM is

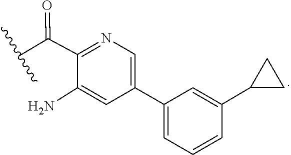 [SMILES: *C(=O)c1ncc(-c2cccc(C3CC3)c2)cc1N]

In some embodiments, DBM is

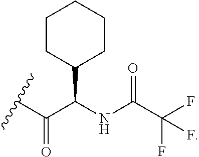 [SMILES: *C(=O)[C@H](NC(=O)C(F)(F)F)C1CCCCC1]

In some embodiments, DBM is

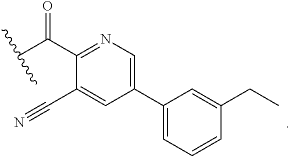 [SMILES: *C(=O)c1ncc(-c2cccc(CC)c2)cc1C#N]

In some embodiments, DBM is

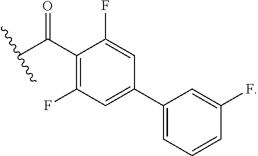 [SMILES: *C(=O)c1c(F)cc(-c2cccc(F)c2)cc1F]

In some embodiments, DBM is

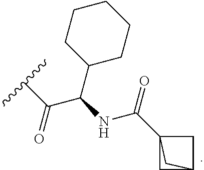 [SMILES: *C(=O)[C@H](NC(=O)C12CC(C1)C2)C1CCCCC1]

In some embodiments, DBM is

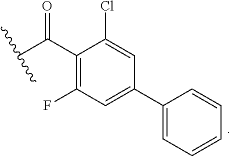 [SMILES: *C(=O)c1c(F)cc(-c2ccccc2)cc1Cl]

In some embodiments, DBM is

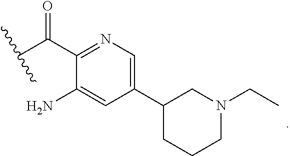 [SMILES: *C(=O)c1ncc(C2CCCN(CC)C2)cc1N]

In some embodiments, DBM is

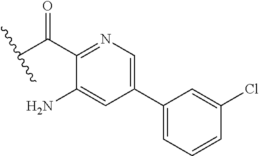 [SMILES: *C(=O)c1ncc(-c2cccc(Cl)c2)cc1N]

In some embodiments, DBM is

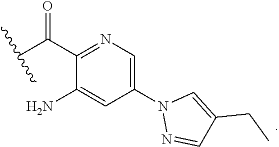 [SMILES: *C(=O)c1ncc(-n2cc(CC)cn2)cc1N]

In some embodiments, DBM is

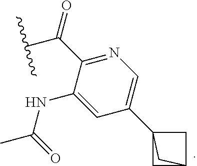 [SMILES: *C(=O)c1ncc(C23CC(C2)C3)cc1NC(C)=O]

In some embodiments, DBM is

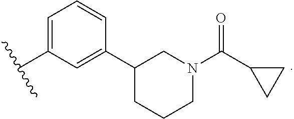 [SMILES: *c1cccc(C2CCCN(C(=O)C3CC3)C2)c1]

In some embodiments, DBM is

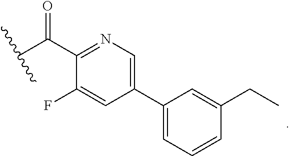 [SMILES: *C(=O)c1ncc(-c2cccc(CC)c2)cc1F]

In some embodiments, DBM is

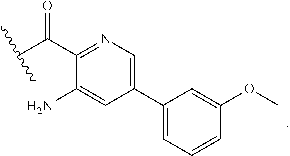 [SMILES: *C(=O)c1ncc(-c2cccc(OC)c2)cc1N]

In some embodiments, DBM is

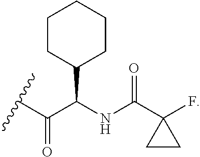 [SMILES: *C(=O)[C@H](NC(=O)C1(F)CC1)C1CCCCC1]

In some embodiments, DBM is

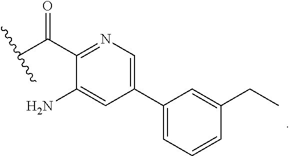 [SMILES: *C(=O)c1ncc(-c2cccc(CC)c2)cc1N]

In some embodiments, DBM is

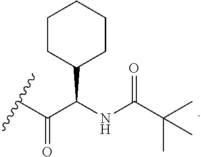 [SMILES: *C(=O)[C@H](NC(=O)C(C)(C)C)C1CCCCC1]

In some embodiments, DBM is

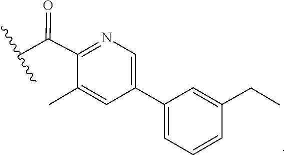 [SMILES: *C(=O)c1ncc(-c2cccc(CC)c2)cc1C]

In some embodiments, DBM is

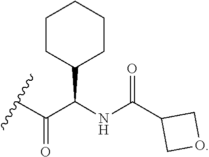 [SMILES: *C(=O)[C@H](NC(=O)C1COC1)C1CCCCC1]

In some embodiments, DBM is

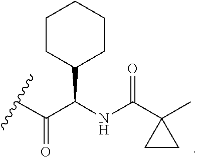 [SMILES: *C(=O)[C@H](NC(=O)C1(C)CC1)C1CCCCC1]

In some embodiments, DBM is

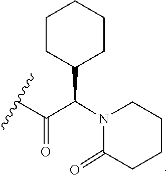 [SMILES: *C(=O)[C@@H](C1CCCCC1)N1CCCCC1=O]

In some embodiments, DBM is

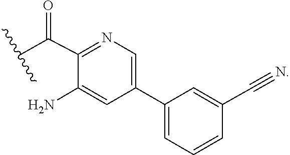 [SMILES: *C(=O)c1ncc(-c2cccc(C#N)c2)cc1N]

In some embodiments, DBM is

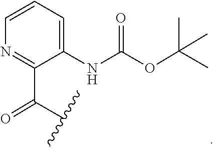 [SMILES: *C(=O)c1ncccc1NC(=O)OC(C)(C)C]

In some embodiments, DBM is

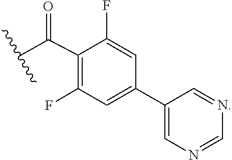 [SMILES: *C(=O)c1c(F)cc(-c2cncnc2)cc1F]

In some embodiments, DBM is

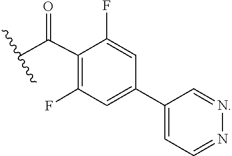 [SMILES: *C(=O)c1c(F)cc(-c2ccnnc2)cc1F]

In some embodiments, DBM is

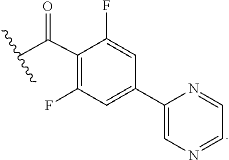 [SMILES: *C(=O)c1c(F)cc(-c2cnccn2)cc1F]

In some embodiments, DBM is

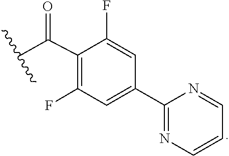 [SMILES: *C(=O)c1c(F)cc(-c2ncccn2)cc1F]

In some embodiments, DBM is

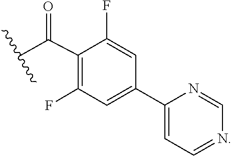 [SMILES: *C(=O)c1c(F)cc(-c2ccncn2)cc1F]

In some embodiments, DBM is

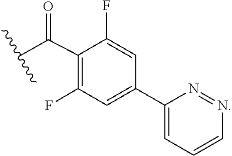 [SMILES: *C(=O)c1c(F)cc(-c2cccnn2)cc1F]

In some embodiments, DBM is

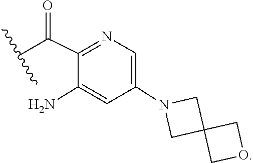 [SMILES: *C(=O)c1ncc(N2CC3(COC3)C2)cc1N]

In some embodiments, DBM is

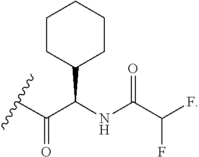 [SMILES: *C(=O)[C@H](NC(=O)C(F)F)C1CCCCC1]

In some embodiments, DBM is

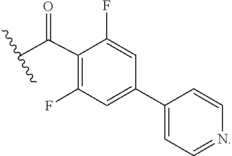 [SMILES: *C(=O)c1c(F)cc(-c2ccncc2)cc1F]

In some embodiments, DBM is

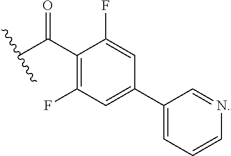 [SMILES: *C(=O)c1c(F)cc(-c2cccnc2)cc1F]

In some embodiments, DBM is

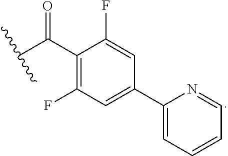 [SMILES: *C(=O)c1c(F)cc(-c2ccccn2)cc1F]

In some embodiments, DBM is

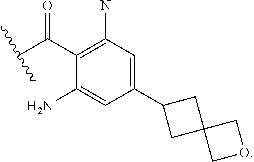 [SMILES: *C(=O)c1c(N)cc(C2CC3(COC3)C2)cc1N]

In some embodiments, DBM is

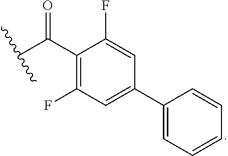 [SMILES: *C(=O)c1c(F)cc(-c2ccccc2)cc1F]

In some embodiments, DBM is

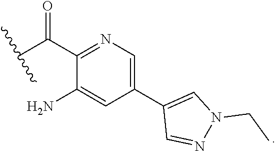 [SMILES: *C(=O)c1ncc(-c2cnn(CC)c2)cc1N]

In some embodiments, DBM is

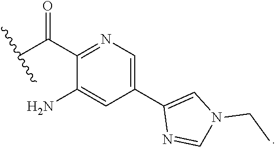 [SMILES: *C(=O)c1ncc(-c2cn(CC)cn2)cc1N]

In some embodiments, DBM is

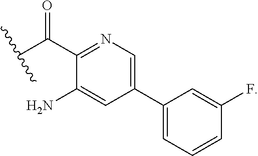 [SMILES: *C(=O)c1ncc(-c2cccc(F)c2)cc1N]

In some embodiments, DBM is

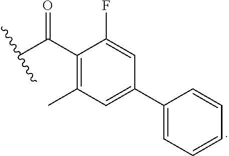 [SMILES: *C(=O)c1c(C)cc(-c2ccccc2)cc1F]

In some embodiments, DBM is

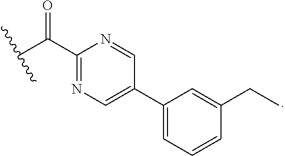 [SMILES: *C(=O)c1ncc(-c2cccc(CC)c2)cn1]

In some embodiments, DBM is

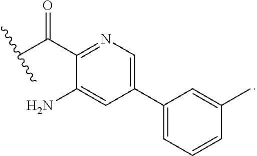 [SMILES: *C(=O)c1ncc(-c2cccc(C)c2)cc1N]

In some embodiments, DBM is

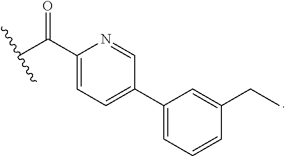 [SMILES: *C(=O)c1ccc(-c2cccc(CC)c2)cn1]

In some embodiments, DBM is

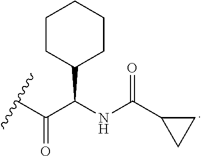 [SMILES: *C(=O)[C@H](NC(=O)C1CC1)C1CCCCC1]

In some embodiments, DBM is

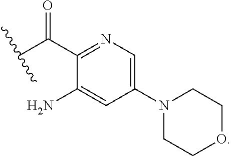 [SMILES: *C(=O)c1ncc(N2CCOCC2)cc1N]

In some embodiments, DBM is

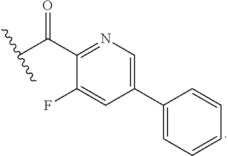 [SMILES: *C(=O)c1ncc(-c2ccccc2)cc1F]

In some embodiments, DBM is

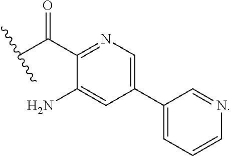 [SMILES: *C(=O)c1ncc(-c2cccnc2)cc1N]

In some embodiments, DBM is

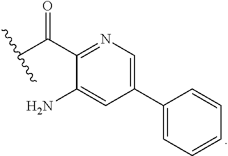 [SMILES: *C(=O)c1ncc(-c2ccccc2)cc1N]

In some embodiments, DBM is

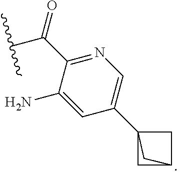 [SMILES: *C(=O)c1ncc(C23CC(C2)C3)cc1N]

In some embodiments, DBM is

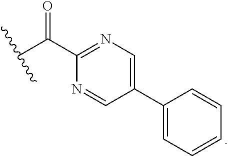 [SMILES: *C(=O)c1ncc(-c2ccccc2)cn1]

In some embodiments, DBM is

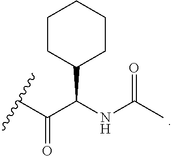 [SMILES: *C(=O)[C@H](NC(C)=O)C1CCCCC1]

In some embodiments, DBM is

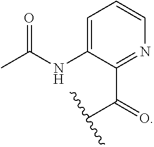 [SMILES: *C(=O)c1ncccc1NC(C)=O]

In some embodiments, DBM is

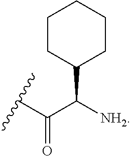 [SMILES: *C(=O)[C@H](N)C1CCCCC1]

In some embodiments, DBM is

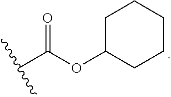 [SMILES: *C(=O)OC1CCCCC1]

In some embodiments, DBM is

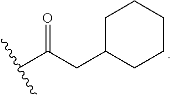 [SMILES: *C(=O)CC1CCCCC1]

In some embodiments, DBM is

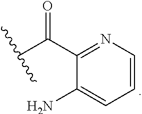 [SMILES: *C(=O)c1ncccc1N]

In some embodiments, DBM is

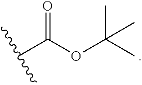 [SMILES: *C(=O)OC(C)(C)C]

In some embodiments, DBM is

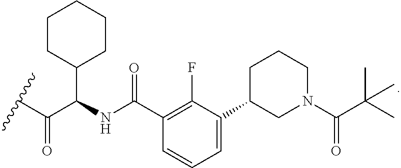 [SMILES: *C(=O)[C@H](NC(=O)c1cccc([C@@H]2CCCN(C(=O)C(C)(C)C)C2)c1F)C1CCCCC1]

In some embodiments, DBM is

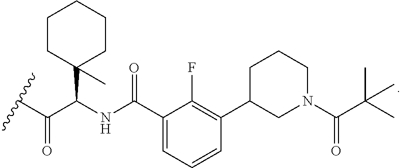 [SMILES: *C(=O)[C@H](NC(=O)c1cccc(C2CCCN(C(=O)C(C)(C)C)C2)c1F)C1(C)CCCCC1]

In some embodiments, DBM is

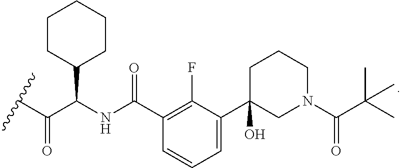 [SMILES: *C(=O)[C@H](NC(=O)c1cccc([C@]2(O)CCCN(C(=O)C(C)(C)C)C2)c1F)C1CCCCC1]

In some embodiments, DBM is

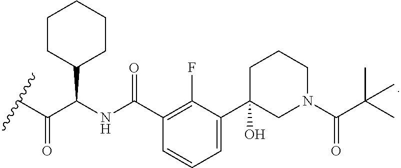 [SMILES: *C(=O)[C@H](NC(=O)c1cccc([C@@]2(O)CCCN(C(=O)C(C)(C)C)C2)c1F)C1CCCCC1]

In some embodiments, DBM is

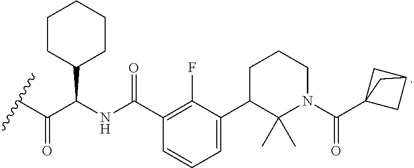 [SMILES: *C(=O)[C@H](NC(=O)c1cccc(C2CCCN(C(=O)C34CC(C3)C4)C2(C)C)c1F)C1CCCCC1]

In some embodiments, DBM is

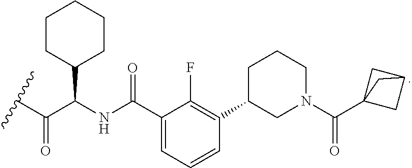 [SMILES: *C(=O)[C@H](NC(=O)c1cccc([C@@H]2CCCN(C(=O)C34CC(C3)C4)C2)c1F)C1CCCCC1]

In some embodiments, DBM is

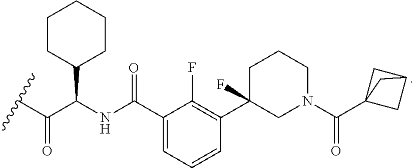 [SMILES: *C(=O)[C@H](NC(=O)c1cccc([C@]2(F)CCCN(C(=O)C34CC(C3)C4)C2)c1F)C1CCCCC1]

In some embodiments, DBM is

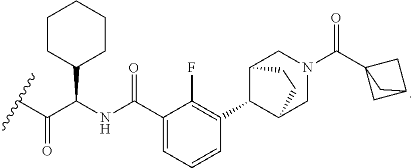 [SMILES: *C(=O)[C@H](NC(=O)c1cccc([C@@H]2[C@@H]3CC[C@H]2CN(C(=O)C24CC(C2)C4)C3)c1F)C1CCCCC1]

In some embodiments, DBM is

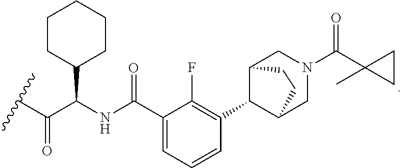 [SMILES: *C(=O)[C@H](NC(=O)c1cccc([C@@H]2[C@@H]3CC[C@H]2CN(C(=O)C2(C)CC2)C3)c1F)C1CCCCC1]

In some embodiments, DBM is

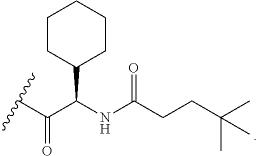 [SMILES: *C(=O)[C@H](NC(=O)CCC(C)(C)C)C1CCCCC1]

In some embodiments, DBM is

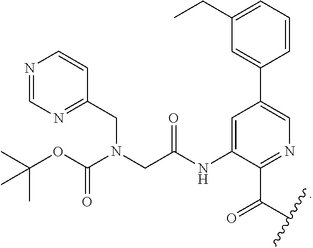 [SMILES: *C(=O)c1ncc(-c2cccc(CC)c2)cc1NC(=O)CN(Cc1ccncn1)C(=O)OC(C)(C)C]

In some embodiments, DBM is

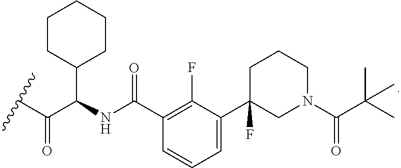 [SMILES: *C(=O)[C@H](NC(=O)c1cccc([C@]2(F)CCCN(C(=O)C(C)(C)C)C2)c1F)C1CCCCC1]

In some embodiments, DBM is

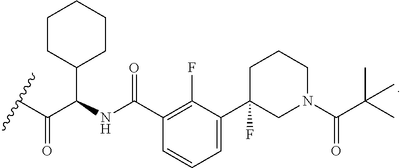 [SMILES: *C(=O)[C@H](NC(=O)c1cccc([C@@]2(F)CCCN(C(=O)C(C)(C)C)C2)c1F)C1CCCCC1]

In some embodiments, DBM is

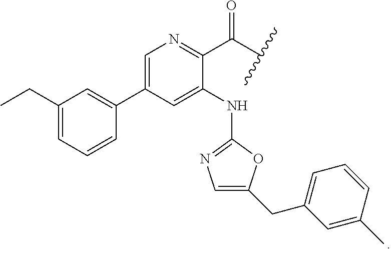 [SMILES: *C(=O)c1ncc(-c2cccc(CC)c2)cc1Nc1ncc(Cc2cccc(C)c2)o1]

In some embodiments, DBM is

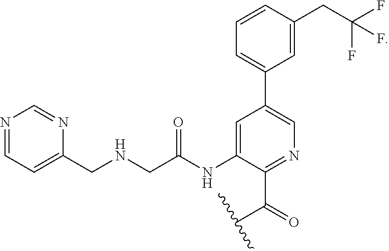 [SMILES: *C(=O)c1ncc(-c2cccc(CC(F)(F)F)c2)cc1NC(=O)CNCc1ccncn1]

In some embodiments, DBM is

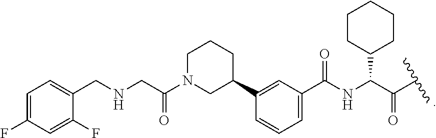 [SMILES: *C(=O)[C@H](NC(=O)c1cccc([C@@H]2CCCN(C(=O)CNCc3ccc(F)cc3F)C2)c1)C1CCCCC1]

In some embodiments, DBM is

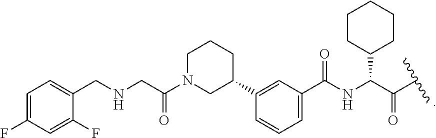 [SMILES: *C(=O)[C@H](NC(=O)c1cccc([C@H]2CCCN(C(=O)CNCc3ccc(F)cc3F)C2)c1)C1CCCCC1]

In some embodiments, DBM is

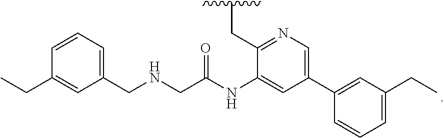 [SMILES: *Cc1ncc(-c2cccc(CC)c2)cc1NC(=O)CNCc1cccc(CC)c1]

In some embodiments, DBM is

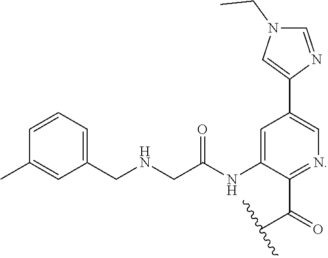 [SMILES: *C(=O)c1ncc(-c2cn(CC)cn2)cc1NC(=O)CNCc1cccc(C)c1]

In some embodiments, DBM is

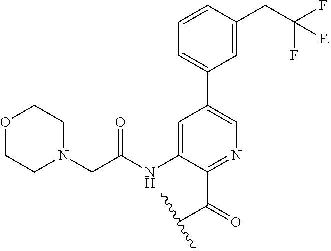 [SMILES: *C(=O)c1ncc(-c2cccc(CC(F)(F)F)c2)cc1NC(=O)CN1CCOCC1]

In some embodiments, DBM is

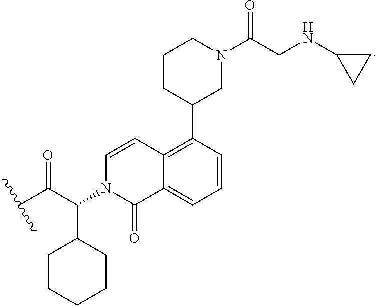 [SMILES: *C(=O)[C@@H](C1CCCCC1)n1ccc2c(C3CCCN(C(=O)CNC4CC4)C3)cccc2c1=O]

In some embodiments, DBM is

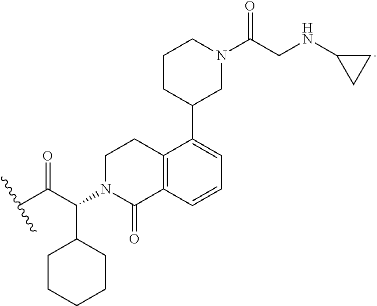 [SMILES: *C(=O)[C@@H](C1CCCCC1)N1CCc2c(cccc2C2CCCN(C(=O)CNC3CC3)C2)C1=O]

In some embodiments, DBM is

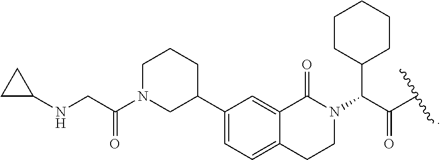 [SMILES: *C(=O)[C@@H](C1CCCCC1)N1CCc2ccc(C3CCCN(C(=O)CNC4CC4)C3)cc2C1=O]

In some embodiments, DBM is

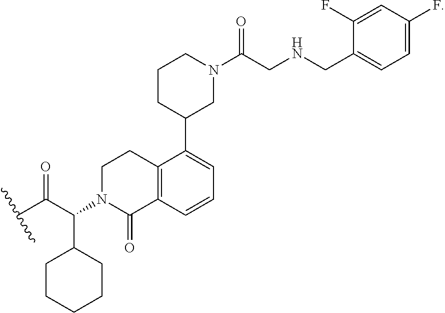 [SMILES: *C(=O)[C@@H](C1CCCCC1)N1CCc2c(cccc2C2CCCN(C(=O)CNCc3ccc(F)cc3F)C2)C1=O]

In some embodiments, DBM is

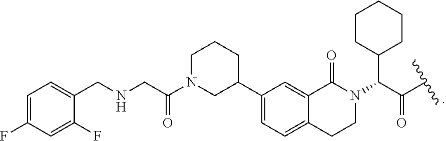 [SMILES: *C(=O)[C@@H](C1CCCCC1)N1CCc2ccc(C3CCCN(C(=O)CNCc4ccc(F)cc4F)C3)cc2C1=O]

In some embodiments, DBM is

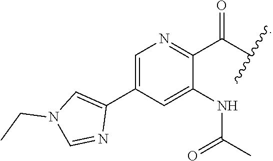 [SMILES: *C(=O)c1ncc(-c2cn(CC)cn2)cc1NC(C)=O]

In some embodiments, DBM is

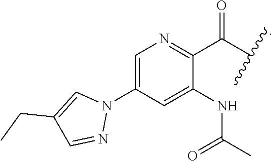 [SMILES: *C(=O)c1ncc(-n2cc(CC)cn2)cc1NC(C)=O]

In some embodiments, DBM is

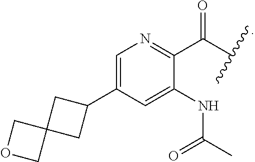 [SMILES: *C(=O)c1ncc(C2CC3(COC3)C2)cc1NC(C)=O]

In some some embodiments, DBM is

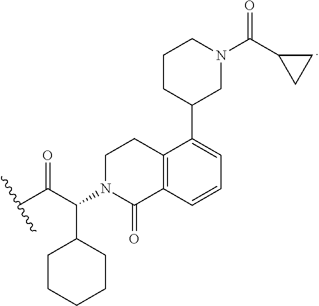 [SMILES: *C(=O)[C@@H](C1CCCCC1)N1CCc2c(cccc2C2CCCN(C(=O)C3CC3)C2)C1=O]

In some embodiments, DBM is

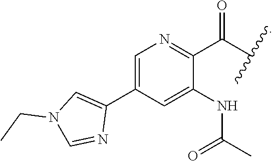 [SMILES: *C(=O)c1ncc(-c2cn(CC)cn2)cc1NC(C)=O]

In some embodiments, DBM is

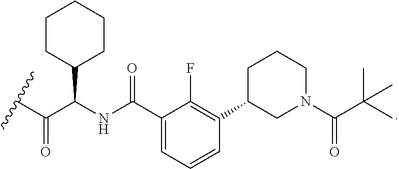 [SMILES: *C(=O)[C@H](NC(=O)c1cccc([C@@H]2CCCN(C(=O)C(C)(C)C)C2)c1F)C1CCCCC1]

In some embodiments, DBM is

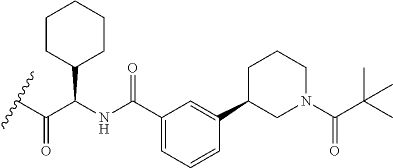 [SMILES: *C(=O)[C@H](NC(=O)c1cccc([C@H]2CCCN(C(=O)C(C)(C)C)C2)c1)C1CCCCC1]

In some embodiments, DBM is selected from those depicted in Table 1, below.

In some embodiments, DBM is selected from those depicted in Table A, below.

### Linker (L)

As defined above and described herein, L is a bivalent moiety that connects PBM to DIM, e.g., DBM. In some embodiments, L is a bivalent moiety that connects PBM to DIM. In some embodiments, L is a bivalent moiety that connects PBM to DBM.

In some embodiments, L is a covalent bond or a bivalent, saturated or partially unsaturated, straight or branched C₁₋₅₀ hydrocarbon chain, wherein 0-6 methylene units of L are independently replaced by -Cy-, —O—, —NR—, —SiR₂—, —Si(OH)R—, —Si(OH)₂—, —P(O)OR—, —P(O)R—, —P(O)NR₂—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, —NRC(O)O—

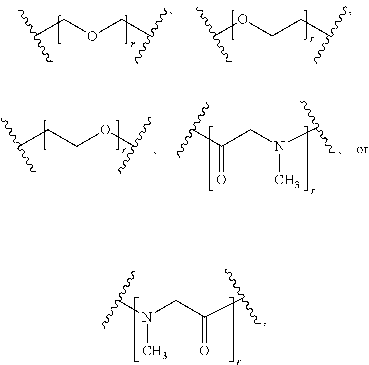

wherein:
each -Cy- is independently an optionally substituted bivalent ring selected from phenylenyl, an 8-10 membered bicyclic arylenyl, a 4-7 membered saturated or partially unsaturated carbocyclylenyl, a 6-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-2 heteroatoms independently selected from nitrogen, oxygen, and sulfur, a 6-11 membered saturated or partially unsaturated spiro heterocyclylenyl having 1-2 heteroatoms independently selected from nitrogen, oxygen, and sulfur, an 8-10 membered bicyclic saturated or partially unsaturated heterocyclylenyl having 1-2 heteroatoms independently selected from nitrogen, oxygen, and sulfur, a 5-6 membered heteroarylenyl having 1-4 heteroatoms independently selected from nitrogen, oxygen, and sulfur, and an 8-10 membered bicyclic heteroarylenyl having 1-5 heteroatoms independently selected from nitrogen, oxygen, and sulfur,
each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same nitrogen are optionally taken together with their intervening atoms to form a 4-7 membered saturated, partially unsaturated, or heteroaryl ring having 0-3 heteroatoms, in addition to the nitrogen, independently selected from nitrogen, oxygen, and sulfur; and
r is 0, 1, 2, 3, 4, 5, 6, 7, 8, 9, or 10.

In some embodiments, each -Cy- is independently an optionally substituted bivalent phenylenyl. In some embodiments, each -Cy- is independently an optionally substituted 8-10 membered bicyclic arylenyl. In some embodiments, each -Cy- is independently an optionally substituted 4-7 membered saturated or partially unsaturated carbocyclylenyl. In some embodiments, each -Cy- is independently an optionally substituted 6-11 membered saturated or partially unsaturated spiro carbocyclylenyl. In some embodiments, each -Cy- is independently an optionally substituted 8-10 membered bicyclic saturated or partially unsaturated carbocyclylenyl. In some embodiments, each -Cy- is independently an optionally substituted 4-7 membered saturated or partially unsaturated heterocyclylenyl having 1-2 heteroatoms independently selected from nitrogen, oxygen, and sulfur. In some embodiments, each -Cy- is independently an optionally substituted 6-11 membered saturated or partially unsaturated spiro heterocyclylenyl having 1-2 heteroatoms independently selected from nitrogen, oxygen, and sulfur. In some embodiments, each -Cy- is independently an optionally substituted 8-10 membered bicyclic saturated or partially unsaturated heterocyclylenyl having 1-2 heteroatoms independently selected from nitrogen, oxygen, and sulfur. In some embodiments, each -Cy- is independently an optionally substituted 5-6 membered heteroarylenyl having 1-4 heteroatoms independently selected from nitrogen, oxygen, and sulfur. In some embodiments, each -Cy- is independently an optionally substituted 8-10 membered bicyclic heteroarylenyl having 1-5 heteroatoms independently selected from nitrogen, oxygen, and sulfur.

In some embodiments, -Cy- is substituted with C₁₋₆ alkyl (e.g., methyl, ethyl, isopropyl). In some embodiments, -Cy- is substituted with oxo. In some embodiments, -Cy- is substituted with halogen. In some embodiments, -Cy- is substituted with fluoro. In some embodiments, -Cy- is substituted with geminal difluoro. In some embodiments, -Cy- is substituted with —OH. In some embodiments, -Cy- is substituted with —NR₂.

In some embodiments, -Cy- is selected from those depicted in Table 1, below.

In some embodiments, -Cy- is selected from those depicted in Table B, below.

In some embodiments, r is 0. In some embodiments, r is 1. In some embodiments, r is 2. In some embodiments, r is 3. In some embodiments, r is 4. In some embodiments, r is 5. In some embodiments, r is 6. In some embodiments, r is 7. In some embodiments, r is 8. In some embodiments, r is 9. In some embodiments, r is 10.

In some embodiments, r is selected from those depicted in Table 1, below.

In some embodiments, r is selected from those depicted in Table B, below.

In some embodiments, L is —O(CH₂)₁₋₁₀—O—. In some embodiments, L is —O(CH₂)₁₋₁₀—NR—. In some embodiments, L is —O(CH₂)₁₋₁₀—C(O)—. In some embodiments, L is —O(CH₂)₁₋₁₀—OCH₂CH₂NR—. In some embodiments, L is —O(CH₂)₁₋₁₀—NRCH₂C(O)—. In some embodiments, L is —O(CH₂)₁₋₁₀—CH₂CH₂OCH₂C(O)—. In some embodiments, L is —O(CH₂)₁₋₁₀—(OCH₂CH₂)₁₋₁₀—NR—. In some embodiments, L is —O(CH₂)₁₋₁₀—CH₂C(O)NRCH₂CH₂NR—. In some embodiments, L is —O(CH₂)₁₋₁₀—NRCH₂C(O)NRCH₂CH₂NR—. In some embodiments, L is —NR(CH₂)₁₋₁₀—O—. In some embodiments, L is —NR(CH₂)₁₋₁₀—NR—. In some embodiments, L is —NR(CH₂)₁₋₁₀—C(O)—. In some embodiments, L is —NR(CH₂)₁₋₁₀—OCH₂CH₂NR—. In some embodiments, L is —NR(CH₂)₁₋₁₀—NRCH₂C(O)—. In some embodiments, L is —NR(CH₂)₁₋₁₀—CH₂CH₂OCH₂C(O)—. In some embodiments, L is —NR(CH₂)₁₋₁₀—(OCH₂CH₂)₁₋₁₀—NR—. In some embodiments, L is —NR(CH₂)₁₋₁₀—CH₂C(O)NRCH₂CH₂NR—. In some embodiments, L is —NR(CH₂)₁₋₁₀—NRCH₂C(O)NRCH₂CH₂NR—. In some embodiments, L is —C(O)(CH₂)₁₋₁₀—O—. In some embodiments, L is —C(O)(CH₂)₁₋₁₀—NR—. In some embodiments, L is —C(O)(CH₂)₁₋₁₀—C(O)—. In some embodiments, L is —C(O)(CH₂)₁₋₁₀—OCH₂CH₂NR—. In some embodiments, L is —C(O)(CH₂)₁₋₁₀—NRCH₂C(O)—. In some embodiments, L is —C(O)(CH₂)₁₋₁₀—CH₂CH₂OCH₂C(O)—. In some embodiments, L is —C(O)(CH₂)₁₋₁₀—(OCH₂CH₂)₁₋₁₀—NR—. In some embodiments, L is —C(O)(CH₂)₁₋₁₀—CH₂C(O)NRCH₂CH₂NR—. In some embodiments, L is —C(O)(CH₂)₁₋₁₀—NRCH₂C(O)NRCH₂CH₂NR—. In some embodiments, L is —NRCH₂CH₂O(CH₂)₁₋₁₀—O—. In some embodiments, L is —NRCH₂CH₂O(CH₂)₁₋₁₀—NR—. In some embodiments, L is —NRCH₂CH₂O(CH₂)₁₋₁₀—C(O)—. In some embodiments, L is —NRCH₂CH₂O(CH₂)₁₋₁₀—OCH₂CH₂NR—. In some embodiments, L is —NRCH₂CH₂O(CH₂)₁₋₁₀—NRCH₂C(O)—. In some embodiments, L is —NRCH₂CH₂O(CH₂)₁₋₁₀—CH₂CH₂OCH₂C(O)—. In some embodiments, L is —NRCH₂CH₂O(CH₂)₁₋₁₀—(OCH₂CH₂)₁₋₁₀—NR—. In some embodiments, L is —NRCH₂CH₂O(CH₂)₁₋₁₀—CH₂C(O)NRCH₂CH₂NR—. In some embodiments, L is —NRCH₂CH₂O(CH₂)₁₋₁₀—NRCH₂C(O)NRCH₂CH₂NR—. In some embodiments, L is —C(O)CH₂NR(CH₂)₁₋₁₀—O—. In some embodiments, L is —C(O)CH₂NR(CH₂)₁₋₁₀—NR—. In some embodiments, L is —C(O)CH₂NR(CH₂)₁₋₁₀—C(O)—. In some embodiments, L is —C(O)CH₂NR(CH₂)₁₋₁₀—OCH₂CH₂NR—. In some embodiments, L is —C(O)CH₂NR(CH₂)₁₋₁₀—NRCH₂C(O)—. In some embodiments, L is —C(O)CH₂NR(CH₂)₁₋₁₀—CH₂CH₂OCH₂C(O)—. In some embodiments, L is —C(O)CH₂NR(CH₂)₁₋₁₀—(OCH₂CH₂)₁₋₁₀—NR—. In some embodiments, L is —C(O)CH₂NR(CH₂)₁₋₁₀—CH₂C(O)NRCH₂CH₂NR—. In some embodiments, L is —C(O)CH₂NR(CH₂)₁₋₁₀—NRCH₂C(O)NRCH₂CH₂NR—. In some embodiments, L is —C(O)CH₂OCH₂CH₂(CH₂)₁₋₁₀—O—. In some embodiments, L is —C(O)CH₂OCH₂CH₂(CH₂)₁₋₁₀—NR—. In some embodiments, L is —C(O)CH₂OCH₂CH₂(CH₂)₁₋₁₀—C(O)—. In some embodiments, L is —C(O)CH₂OCH₂CH₂(CH₂)₁₋₁₀—OCH₂CH₂NR—. In some embodiments, L is —C(O)CH₂OCH₂CH₂(CH₂)₁₋₁₀—NRCH₂C(O)—. In some embodiments, L is —C(O)CH₂OCH₂CH₂(CH₂)₁₋₁₀—CH₂CH₂OCH₂C(O)—. In some embodiments, L is —C(O)CH₂OCH₂CH₂(CH₂)₁₋₁₀—(OCH₂CH₂)₁₋₁₀—NR—. In some embodiments, L is —C(O)CH₂OCH₂CH₂(CH₂)₁₋₁₀—CH₂C(O)NRCH₂CH₂NR—. In some embodiments, L is —C(O)CH₂OCH₂CH₂(CH₂)₁₋₁₀—NRCH₂C(O)NRCH₂CH₂NR—. In some embodiments, L is —NR(CH₂CH₂O)₁₋₁₀—(CH₂)₁₋₁₀—O—. In some embodiments, L is —NR(CH₂CH₂O)₁₋₁₀—(CH₂)₁₋₁₀—NR—. In some embodiments, L is —NR(CH₂CH₂O)₁₋₁₀—(CH₂)₁₋₁₀—C(O)—. In some embodiments, L is —NR(CH₂CH₂O)₁₋₁₀—(CH₂)₁₋₁₀—OCH₂CH₂NR—. In some embodiments, L is —NR(CH₂CH₂O)₁₋₁₀—(CH₂)₁₋₁₀—NRCH₂C(O)—. In some embodiments, L is —NR(CH₂CH₂O)₁₋₁₀—(CH₂)₁₋₁₀—CH₂CH₂OCH₂C(O)—. In some embodiments, L is —NR(CH₂CH₂O)₁₋₁₀—(CH₂)₁₋₁₀—(OCH₂CH₂)₁₋₁₀—NR—. In some embodiments, L is —NR(CH₂CH₂O)₁₋₁₀—(CH₂)₁₋₁₀—CH₂C(O)NRCH₂CH₂NR—. In some embodiments, L is —NR(CH₂CH₂O)₁₋₁₀—(CH₂)₁₋₁₀—NRCH₂C(O)NRCH₂CH₂NR—. In some embodiments, L is —NRCH₂CH₂NRC(O)CH₂(CH₂)₁₋₁₀—O—. In some embodiments, L is —NRCH₂CH₂NRC(O)CH₂(CH₂)₁₋₁₀—NR—. In some embodiments, L is —NRCH₂CH₂NRC(O)CH₂(CH₂)₁₋₁₀—C(O)—. In some embodiments, L is —NRCH₂CH₂NRC(O)CH₂(CH₂)₁₋₁₀—NR—. In some embodiments, L is —NRCH₂CH₂NRC(O)CH₂(CH₂)₁₋₁₀—NRCH₂C(O)—. In some embodiments, L is —NRCH₂CH₂NRC(O)CH₂(CH₂)₁₋₁₀—CH₂CH₂OCH₂C(O)—. In some embodiments, L is —NRCH₂CH₂NRC(O)CH₂(CH₂)₁₋₁₀—(OCH₂CH₂)₁₋₁₀—NR—. In some embodiments, L is —NRCH₂CH₂NRC(O)CH₂(CH₂)₁₋₁₀—CH₂C(O)NRCH₂CH₂NR—. In some embodiments, L is —NRCH₂CH₂NRC(O)CH₂(CH₂)₁₋₁₀—NRCH₂C(O)NRCH₂CH₂NR—. In some embodiments, L is —NRCH₂CH₂NRC(O)CH₂NR(CH₂)₁₋₁₀—O—. In some embodiments, L is —NRCH₂CH₂NRC(O)CH₂NR(CH₂)₁₋₁₀—NR—. In some embodiments, L is —NRCH₂CH₂NRC(O)CH₂NR(CH₂)₁₋₁₀—C(O)—. In some embodiments, L is —NRCH₂CH₂NRC(O)CH₂NR(CH₂)₁₋₁₀—OCH₂CH₂NR—. In some embodiments, L is —NRCH₂CH₂NRC(O)CH₂NR(CH₂)₁₋₁₀—NRCH₂C(O)—. In some embodiments, L is —NRCH₂CH₂NRC(O)CH₂NR(CH₂)₁₋₁₀—CH₂CH₂OCH₂C(O)—. In some embodiments, L is —NRCH₂CH₂NRC(O)CH₂NR(CH₂)₁₋₁₀—(OCH₂CH₂)₁₋₁₀—NR—. In some embodiments, L is —NRCH₂CH₂NRC(O)CH₂NR(CH₂)₁₋₁₀—CH₂C(O)NRCH₂CH₂NR—. In some embodiments, L is —NRCH₂CH₂NRC(O)CH₂NR(CH₂)₁₋₁₀—NRCH₂C(O)NRCH₂CH₂NR—.

In some embodiments, L is —(OCH₂CH₂)₁₋₁₀—O—. In some embodiments, L is —(OCH₂CH₂)₁₋₁₀-Cy-. In some embodiments, L is —(OCH₂CH₂)₁₋₁₀—NR—. In some embodiments, L is —(OCH₂CH₂)₁₋₁₀—C(O)—. In some embodiments, L is —(OCH₂CH₂)₁₋₁₀—NRCH₂C(O)—. In some embodiments, L is —(OCH₂CH₂)₁₋₁₀—NR—. In some embodiments, L is —(OCH₂CH₂)₁₋₁₀—NRCH₂C(O)NRCH₂CH₂NR—. In some embodiments, L is —NRCH₂CH₂(OCH₂CH₂)₁₋₁₀—O—. In some embodiments, L is —NRCH₂CH₂(OCH₂CH₂)₁₋₁₀-Cy-. In some embodiments, L is —NRCH₂CH₂(OCH₂CH₂)₁₋₁₀—NR—. In some embodiments, L is —NRCH₂CH₂(OCH₂CH₂)₁₋₁₀—C(O)—. In some embodiments, L is —NRCH₂CH₂(OCH₂CH₂)₁₋₁₀—NRCH₂C(O)—. In some embodiments, L is —NRCH₂CH₂(OCH₂CH₂)₁₋₁₀—NR—. In some embodiments, L is —NRCH₂CH₂(OCH₂CH₂)₁₋₁₀—NRCH₂C(O)NRCH₂CH₂NR—. In some embodiments, L is —C(O)CH₂CH₂(OCH₂CH₂)₁₋₁₀—O—. In some embodiments, L is —C(O)CH₂CH₂(OCH₂CH₂)₁₋₁₀-Cy-. In some embodiments, L is —C(O)CH₂CH₂(OCH₂CH₂)₁₋₁₀—NR—. In some embodiments, L is —C(O)CH₂CH₂(OCH₂CH₂)₁₋₁₀—C(O)—. In some embodiments, L is —C(O)CH₂CH₂(OCH₂CH₂)₁₋₁₀—NRCH₂C(O)—. In some embodiments, L is —C(O)CH₂CH₂(OCH₂CH₂)₁₋₁₀—NR—. In some embodiments, L is —C(O)CH₂CH₂(OCH₂CH₂)₁₋₁₀—NRCH₂C(O)NRCH₂CH₂NR—. In some embodiments, L is —C(O)CH₂OCH₂CH₂(OCH₂CH₂)₁₋₁₀—O—. In some embodiments, L is —C(O)CH₂OCH₂CH₂(OCH₂CH₂)₁₋₁₀-Cy-. In some embodiments, L is —C(O)CH₂OCH₂CH₂(OCH₂CH₂)₁₋₁₀—NR—. In some embodiments, L is —C(O)CH₂OCH₂CH₂(OCH₂CH₂)₁₋₁₀—C(O)—. In some embodiments, L is —C(O)CH₂OCH₂CH₂(OCH₂CH₂)₁₋₁₀—NRCH₂C(O)—. In some embodiments, L is —C(O)CH₂OCH₂CH₂(OCH₂CH₂)₁₋₁₀—NR—. In some embodiments, L is —C(O)CH₂OCH₂CH₂(OCH₂CH₂)₁₋₁₀—NRCH₂C(O)NRCH₂CH₂NR—. In some embodiments, L is —C(O)CH₂NRCH₂CH₂(OCH₂CH₂)₁₋₁₀—O—. In some embodiments, L is —C(O)CH₂NRCH₂CH₂(OCH₂CH₂)₁₋₁₀-Cy-. In some embodiments, L is —C(O)CH₂NRCH₂CH₂(OCH₂CH₂)₁₋₁₀—NR—. In some embodiments, L is —C(O)CH₂NRCH₂CH₂(OCH₂CH₂)₁₋₁₀—C(O)—. In some embodiments, L is —C(O)CH₂NRCH₂CH₂(OCH₂CH₂)₁₋₁₀—NRCH₂C(O)—. In some embodiments, L is —C(O)CH₂NRCH₂CH₂(OCH₂CH₂)₁₋₁₀—NR—. In some embodiments, L is —C(O)CH₂NRCH₂CH₂(OCH₂CH₂)₁₋₁₀—NRCH₂C(O)NRCH₂CH₂NR—. In some embodiments, L is —NRCH₂CH₂NRC(O)CH₂(OCH₂CH₂)₁₋₁₀—O—. In some embodiments, L is —NRCH₂CH₂NRC(O)CH₂(OCH₂CH₂)₁₋₁₀-Cy-. In some embodiments, L is —NRCH₂CH₂NRC(O)CH₂(OCH₂CH₂)₁₋₁₀—NR—. In some embodiments, L is —NRCH₂CH₂NRC(O)CH₂(OCH₂CH₂)₁₋₁₀—C(O)—. In some embodiments, L is —NRCH₂CH₂NRC(O)CH₂(OCH₂CH₂)₁₋₁₀—NRCH₂C(O)—. In some embodiments, L is —NRCH₂CH₂NRC(O)CH₂(OCH₂CH₂)₁₋₁₀—NR—. In some embodiments, L is —NRCH₂CH₂NRC(O)CH₂(OCH₂CH₂)₁₋₁₀—NRCH₂C(O)NRCH₂CH₂NR—. In some embodiments, L is —NRCH₂CH₂NRC(O)CH₂NRCH₂CH₂(OCH₂CH₂)₁₋₁₀—O—. In some embodiments, L is —NRCH₂CH₂NRC(O)CH₂NRCH₂CH₂(OCH₂CH₂)₁₋₁₀-Cy-. In some embodiments, L is —NRCH₂CH₂NRC(O)CH₂NRCH₂CH₂(OCH₂CH₂)₁₋₁₀—NR—. In some embodiments, L is —NRCH₂CH₂NRC(O)CH₂NRCH₂CH₂(OCH₂CH₂)₁₋₁₀—C(O)—. In some embodiments, L is —NRCH₂CH₂NRC(O)CH₂NRCH₂CH₂(OCH₂CH₂)₁₋₁₀—NRCH₂C(O)—. In some embodiments, L is —NRCH₂CH₂NRC(O)CH₂NRCH₂CH₂(OCH₂CH₂)₁₋₁₀—NR—. In some embodiments, L is —NRCH₂CH₂NRC(O)CH₂NRCH₂CH₂(OCH₂CH₂)₁₋₁₀—NRCH₂C(O)NRCH₂CH₂NR—. In some embodiments, L is —O(CH₂CH₂O)₁₋₁₀—CH₂CH₂O—. In some embodiments, L is -Cy-(CH₂CH₂O)₁₋₁₀—CH₂CH₂O—. In some embodiments, L is —NR(CH₂CH₂O)₁₋₁₀—CH₂CH₂O—. In some embodiments, L is —C(O)(CH₂CH₂O)₁₋₁₀—CH₂CH₂O—. In some embodiments, L is —C(O)CH₂NR(CH₂CH₂O)₁₋₁₀—CH₂CH₂O—. In some embodiments, L is —NRCH₂CH₂O(CH₂CH₂O)₁₋₁₀—CH₂CH₂O—. In some embodiments, L is —NRCH₂CH₂NRC(O)CH₂NR(CH₂CH₂O)₁₋₁₀—CH₂CH₂O—. In some embodiments, L is —O(CH₂CH₂O)₁₋₁₀—CH₂CH₂NR—. In some embodiments, L is -Cy-(CH₂CH₂O)₁₋₁₀—CH₂CH₂NR—. In some embodiments, L is —NR(CH₂CH₂O)₁₋₁₀—CH₂CH₂NR—. In some embodiments, L is —C(O)(CH₂CH₂O)₁₋₁₀—CH₂CH₂NR—. In some embodiments, L is —C(O)CH₂NR(CH₂CH₂O)₁₋₁₀—CH₂CH₂NR—. In some embodiments, L is —NRCH₂CH₂O(CH₂CH₂O)₁₋₁₀—CH₂CH₂NR—. In some embodiments, L is —NRCH₂CH₂NRC(O)CH₂NR(CH₂CH₂O)₁₋₁₀—CH₂CH₂NR—. In some embodiments, L is —O(CH₂CH₂O)₁₋₁₀—CH₂CH₂C(O)—. In some embodiments, L is -Cy-(CH₂CH₂O)₁₋₁₀—CH₂CH₂C(O)—. In some embodiments, L is —NR(CH₂CH₂O)₁₋₁₀—CH₂CH₂C(O)—. In some embodiments, L is —C(O)(CH₂CH₂O)₁₋₁₀—CH₂CH₂C(O)—. In some embodiments, L is —C(O)CH₂NR(CH₂CH₂O)₁₋₁₀—CH₂CH₂C(O)—. In some embodiments, L is —NRCH₂CH₂O(CH₂CH₂O)₁₋₁₀—CH₂CH₂C(O)—. In some embodiments, L is —NRCH₂CH₂NRC(O)CH₂NR(CH₂CH₂O)₁₋₁₀—CH₂CH₂C(O)—. In some embodiments, L is —O(CH₂CH₂O)₁₋₁₀—CH₂CH₂OCH₂C(O)—. In some embodiments, L is -Cy-(CH₂CH₂O)₁₋₁₀—CH₂CH₂OCH₂C(O)—. In some embodiments, L is —NR(CH₂CH₂O)₁₋₁₀—CH₂CH₂OCH₂C(O)—. In some embodiments, L is —C(O)(CH₂CH₂O)₁₋₁₀—CH₂CH₂OCH₂C(O)—. In some embodiments, L is —C(O)CH₂NR(CH₂CH₂O)₁₋₁₀—CH₂CH₂OCH₂C(O)—. In some embodiments, L is —NRCH₂CH₂O(CH₂CH₂O)₁₋₁₀—CH₂CH₂OCH₂C(O)—. In some embodiments, L is —NRCH₂CH₂NRC(O)CH₂NR(CH₂CH₂O)₁₋₁₀—CH₂CH₂OCH₂C(O)—. In some embodiments, L is —O(CH₂CH₂O)₁₋₁₀—CH₂CH₂NRCH₂C(O)—. In some embodiments, L is -Cy-(CH₂CH₂O)₁₋₁₀—CH₂CH₂NRCH₂C(O)—. In some embodiments, L is —NR(CH₂CH₂O)₁₋₁₀—CH₂CH₂NRCH₂C(O)—. In some embodiments, L is —C(O)(CH₂CH₂O)₁₋₁₀—CH₂CH₂NRCH₂C(O)—. In some embodiments, L is —C(O)CH₂NR(CH₂CH₂O)₁₋₁₀—CH₂CH₂NRCH₂C(O)—. In some embodiments, L is —NRCH₂CH₂O(CH₂CH₂O)₁₋₁₀—CH₂CH₂NRCH₂C(O)—. In some embodiments, L is —NRCH₂CH₂NRC(O)CH₂NR(CH₂CH₂O)₁₋₁₀—CH₂CH₂NRCH₂C(O)—. In some embodiments, L is —O(CH₂CH₂O)₁₋₁₀—CH₂C(O)NRCH₂CH₂NR—. In some embodiments, L is -Cy-(CH₂CH₂O)₁₋₁₀—CH₂C(O)NRCH₂CH₂NR—. In some embodiments, L is —NR(CH₂CH₂O)₁₋₁₀—CH₂C(O)NRCH₂CH₂NR—. In some embodiments, L is —C(O)(CH₂CH₂O)₁₋₁₀—CH₂C(O)NRCH₂CH₂NR—. In some embodiments, L is —C(O)CH₂NR(CH₂CH₂O)₁₋₁₀—CH₂C(O)NRCH₂CH₂NR—. In some embodiments, L is —NRCH₂CH₂O(CH₂CH₂O)₁₋₁₀—CH₂C(O)NRCH₂CH₂NR—. In some embodiments, L is —NRCH₂CH₂NRC(O)CH₂NR(CH₂CH₂O)₁₋₁₀—CH₂C(O)NRCH₂CH₂NR—. In some embodiments, L is —O(CH₂CH₂O)₁₋₁₀—CH₂CH₂NRCH₂C(O)NRCH₂CH₂NR—. In some embodiments, L is -Cy-(CH₂CH₂O)₁₋₁₀—CH₂CH₂NRCH₂C(O)NRCH₂CH₂NR—. In some embodiments, L is —NR(CH₂CH₂O)₁₋₁₀—CH₂CH₂NRCH₂C(O)NRCH₂CH₂NR—. In some embodiments, L is —C(O)(CH₂CH₂O)₁₋₁₀—CH₂CH₂NRCH₂C(O)NRCH₂CH₂NR—. In some embodiments, L is —C(O)CH₂NR(CH₂CH₂O)₁₋₁₀—CH₂CH₂NRCH₂C(O)NRCH₂CH₂NR—. In some embodiments, L is —NRCH₂CH₂O(CH₂CH₂O)₁₋₁₀—CH₂CH₂NRCH₂C(O)NRCH₂CH₂NR—. In some embodiments, L is —NRCH₂CH₂NRC(O)CH₂NR(CH₂CH₂O)₁₋₁₀—CH₂CH₂NRCH₂C(O)NRCH₂CH₂NR—.

In some embodiments, L is —NRCH₂CH₂-Cy-CH₂O—. In some embodiments, L is —OCH₂-Cy-CH₂CH₂NR—. In some embodiments, L is —NRCH₂CH₂-Cy-CH₂CH₂NR—. In some embodiments, L is —NRCH₂CH₂-Cy-CH₂C(O)—. In some embodiments, L is —C(O)CH₂-Cy-CH₂CH₂NR—. In some embodiments, L is —C(O)CH₂NRCH₂CH₂-Cy-CH₂NR—. In some embodiments, L is —NRCH₂-Cy-CH₂CH₂NRCH₂C(O)—. In some embodiments, L is —C(O)CH₂CH₂-Cy-CH₂C(O)—. In some embodiments, L is —C(O)CH₂-Cy-CH₂CH₂C(O)—. In some embodiments, L is —C(O)CH₂CH₂-Cy-CH₂CH₂NR—. In some embodiments, L is —NRCH₂CH₂-Cy-CH₂CH₂C(O)—. In some embodiments, L is —NRCH₂CH₂NRC(O)CH₂NRCH₂CH₂-Cy-CH₂NR—. In some embodiments, L is —NRCH₂-Cy-CH₂CH₂NRCH₂C(O)NRCH₂CH₂NR—. In some embodiments, L is —NRCH₂CH₂-Cy-SO₂NR—. In some embodiments, L is —NRSO₂-Cy-CH₂CH₂NR—. In some embodiments, L is -Cy-(OCH₂CH₂)₁₋₁₀—NR—. In some embodiments, L is -Cy-(OCH₂CH₂)₁₋₁₀—O—. In some embodiments, L is —O(CH₂CH₂O)₁₋₁₀-Cy-. In some embodiments, L is —NR(CH₂CH₂O)₁₋₁₀-Cy-. In some embodiments, L is —(OCH₂CH₂)₁₋₁₀-Cy-C(O)—. In some embodiments, L is —C(O)-Cy-(CH₂CH₂O)₁₋₁₀—. In some embodiments, L is —NR(CH₂CH₂O)₁₋₁₀-Cy-NRCH₂C(O)—. In some embodiments, L is —C(O)CH₂NR-Cy-(OCH₂CH₂)₁₋₁₀—NR—. In some embodiments, L is —C(O)CH₂-Cy-NRCH₂C(O)—. In some embodiments, L is —C(O)CH₂NR-Cy-CH₂C(O)—. In some embodiments, L is —NRCH₂CH₂NRC(O)CH₂-Cy-C(O)—. In some embodiments, L is —C(O)-Cy-CH₂C(O)NRCH₂CH₂NR—. In some embodiments, L is —NR(CH₂CH₂O)₁₋₁₀-Cy-CH₂CH₂C(O)—. In some embodiments, L is —C(O)CH₂CH₂-Cy-(OCH₂CH₂)₁₋₁₀—NR—. In some embodiments, L is —OCH₂CH₂-Cy-O—. In some embodiments, L is —O-Cy-CH₂CH₂O—. In some embodiments, L is —NRCH₂CH₂O-Cy-NR—. In some embodiments, L is —NR-Cy-OCH₂CH₂NR—. In some embodiments, L is —C(O)CH₂-Cy-C(O)—. In some embodiments, L is —C(O)-Cy-CH₂C(O)—. In some embodiments, L is —NRCH₂CH₂O-Cy-NRCH₂C(O)NRCH₂CH₂NR—. In some embodiments, L is —NRCH₂CH₂NRC(O)CH₂NR-Cy-OCH₂CH₂NR—. In some embodiments, L is —NRCH₂CH₂NRC(O)CH₂-Cy-NRCH₂C(O)NRCH₂CH₂NR—. In some embodiments, L is —NRCH₂CH₂NRC(O)CH₂NR-Cy-CH₂C(O)NRCH₂CH₂NR—.

In some embodiments, L is -Cy-CH₂-Cy-. In some embodiments, L is -Cy-CH₂-Cy-CH₂C(O)—. In some embodiments, L is —C(O)CH₂-Cy-CH₂-Cy-. In some embodiments, L is -Cy-CH₂-Cy-CH₂CH₂O—. In some embodiments, L is —OCH₂CH₂-Cy-CH₂-Cy-. In some embodiments, L is -Cy-CH₂-Cy-CH₂CH₂NR—. In some embodiments, L is —NRCH₂CH₂-Cy-CH₂-Cy-. In some embodiments, L is -Cy-CH₂-Cy-CH₂C(O)NRCH₂CH₂NR—. In some embodiments, L is —NRCH₂CH₂NRC(O)CH₂-Cy-CH₂-Cy-. In some embodiments, L is —O—CH₂CH₂-Cy-CH₂-Cy-. In some embodiments, L is —C(O)CH₂-Cy-CH₂-Cy-. In some embodiments, L is —C(O)CH₂-Cy-CH₂-Cy-CH₂C(O)—. In some embodiments, L is —C(O)CH₂-Cy-CH₂-Cy-CH₂CH₂OCH₂C(O)—. In some embodiments, L is —C(O)CH₂OCH₂CH₂-Cy-CH₂-Cy-CH₂C(O)—. In some embodiments, L is —NRCH₂CH₂NRC(O)CH₂-Cy-CH₂-Cy-. In some embodiments, L is -Cy-CH₂-Cy-C(O)—. In some embodiments, L is —C(O)-Cy-CH₂-Cy-. In some embodiments, L is -Cy-CH₂-Cy-NR—. In some embodiments, L is —NR-Cy-CH₂-Cy-. In some embodiments, L is —OCH₂CH₂-Cy-CH₂-Cy-NR—. In some embodiments, L is —NR-Cy-CH₂-Cy-CH₂CH₂O—. In some embodiments, L is —NR-Cy-CH₂-Cy-C(O)CH₂—. In some embodiments, L is —NRCH₂CH₂NRC(O)CH₂-Cy-CH₂C(O)—. In some embodiments, L is —C(O)CH₂-Cy-CH₂C(O)NRCH₂CH₂NR—. In some embodiments, L is —C(O)-Cy-CH₂NRCH₂C(O)—. In some embodiments, L is —NR-Cy-CH₂NRCH₂C(O)NRCH₂CH₂NR—. In some embodiments, L is —NRCH₂CH₂NRC(O)CH₂NRCH₂-Cy-NR—.

In some embodiments, L is a covalent bond.

In some embodiments, L is —C(O)—CH₂-Cy-CH₂-Cy-. In some embodiments, L is —C(O)—CH₂-Cy-O-Cy-. In some embodiments, L is —C(O)—CH₂-Cy-. In some embodiments, wherein L is -Cy-CH₂-Cy-. In some embodiments, L is -Cy-O-Cy-.

Without limitation, the point of attachment of L to PBM and DIM can be, for example when L is

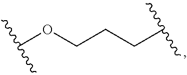 [SMILES: *CCCO*]

either

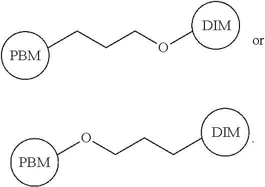

In some embodiments, L is

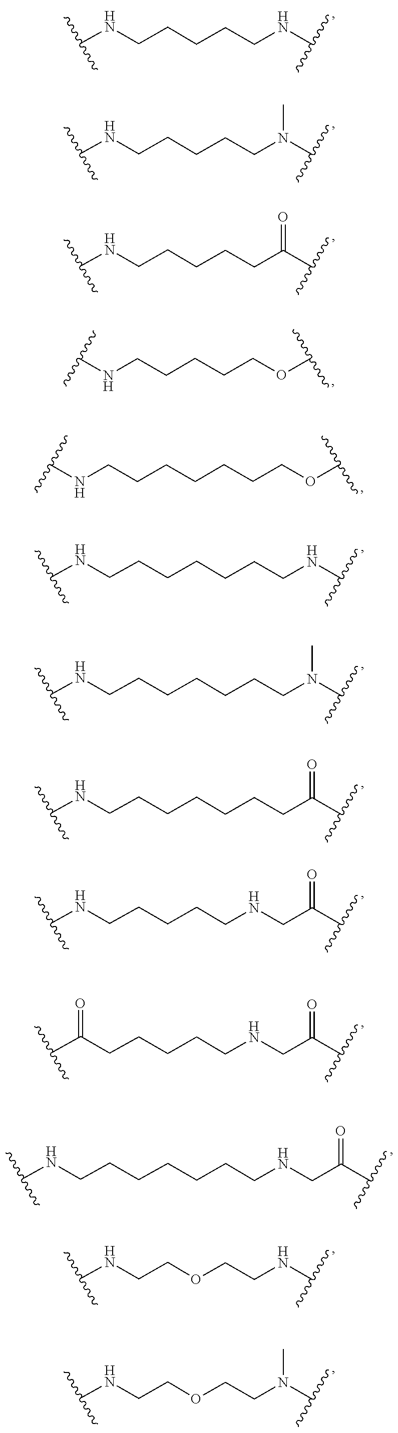

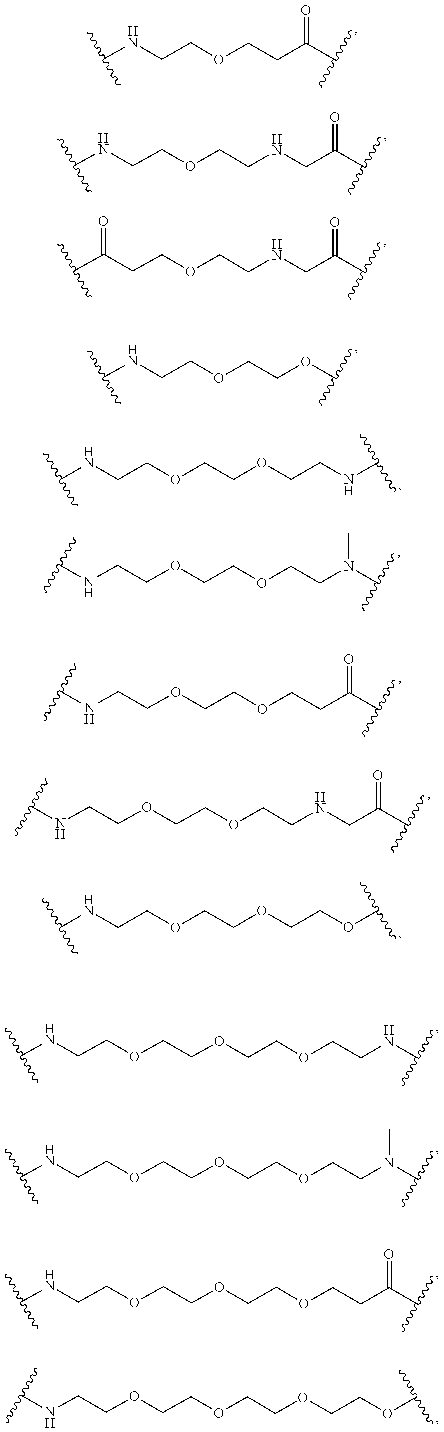

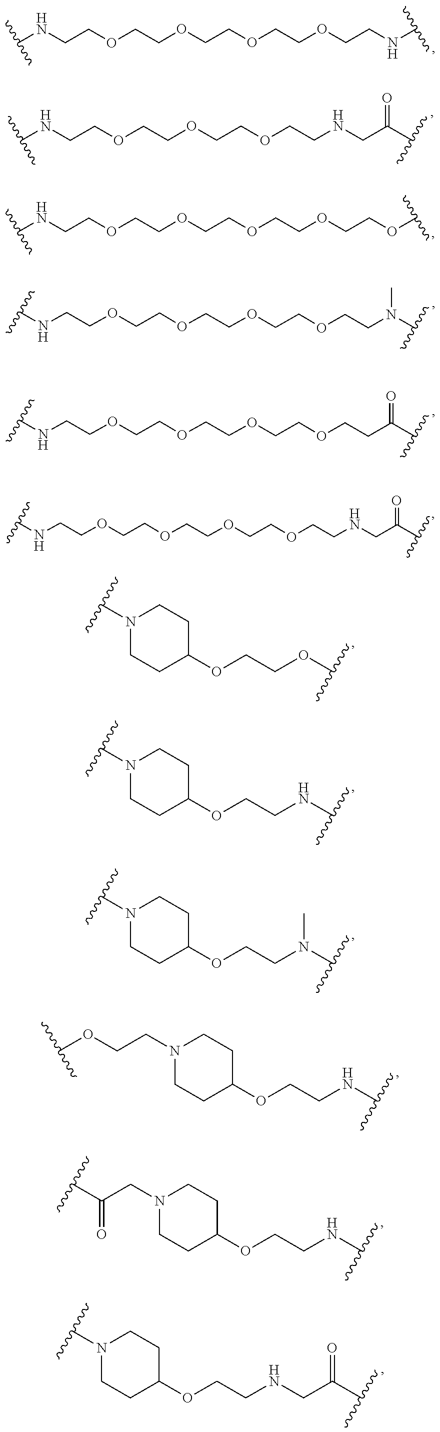

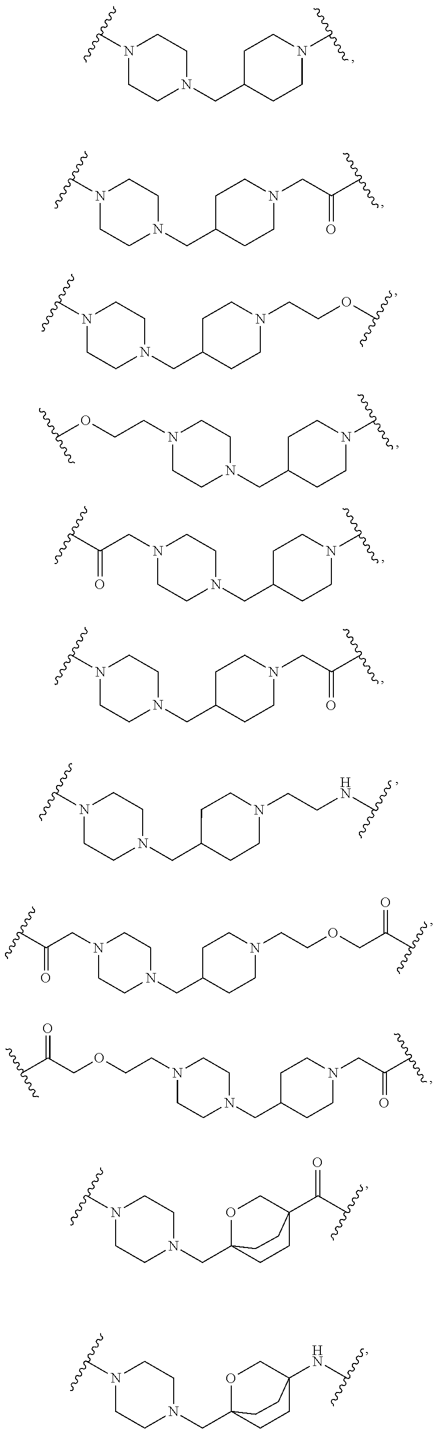

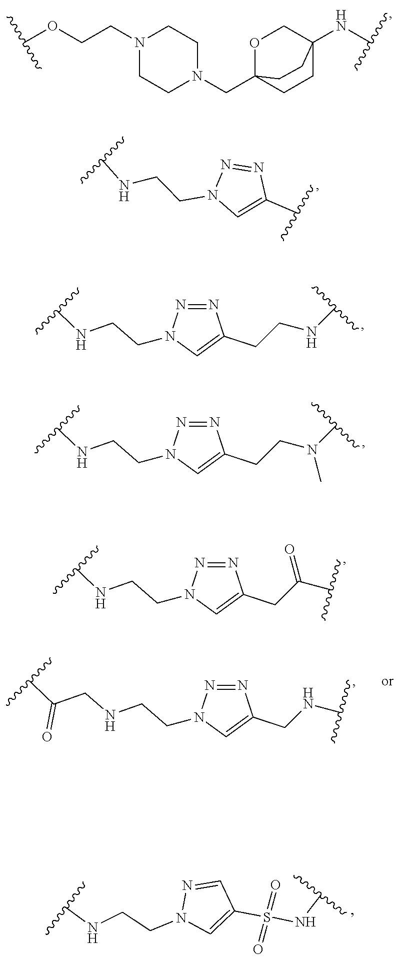

In some embodiments, L is

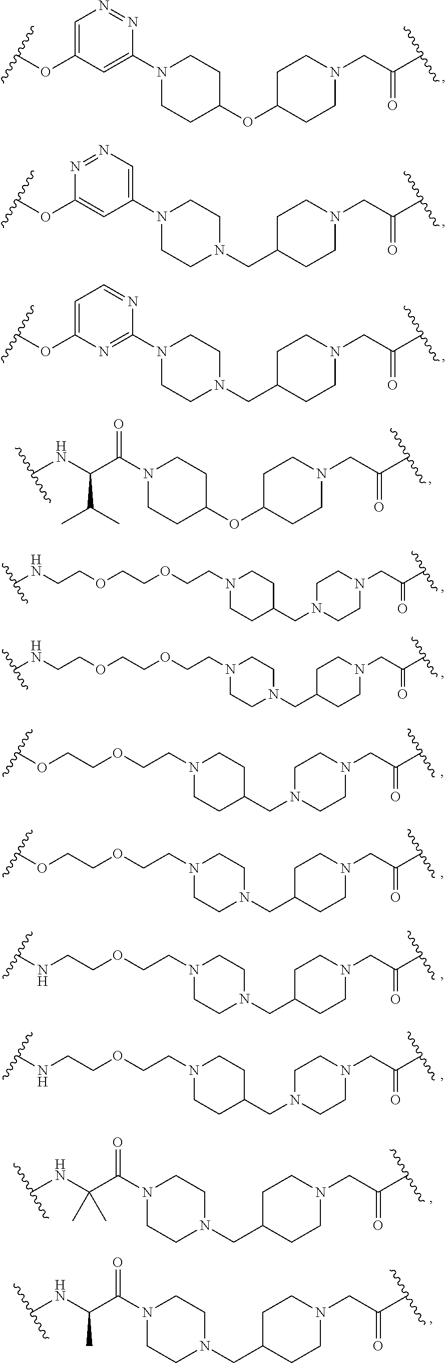

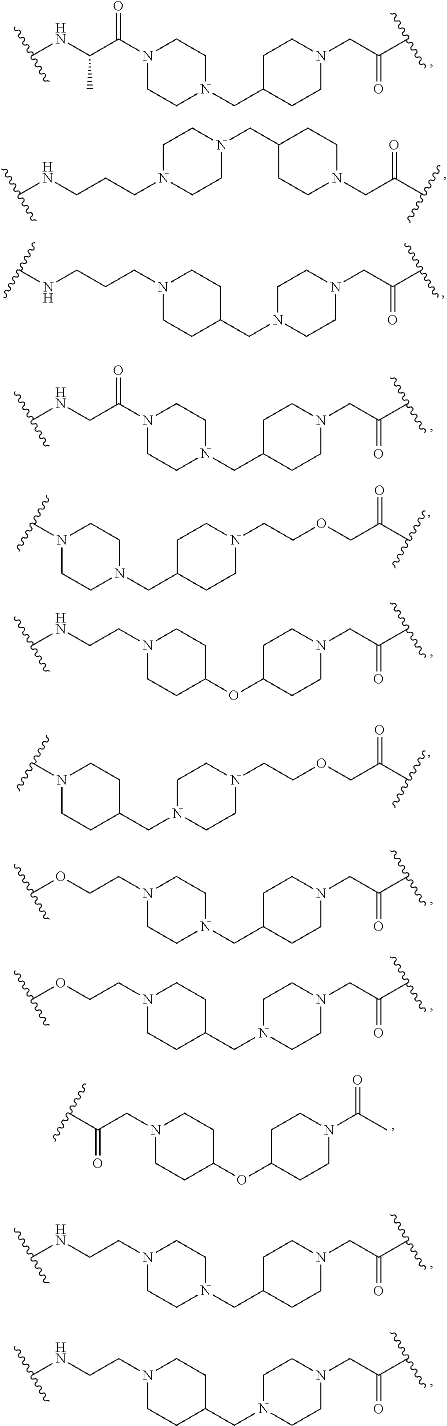

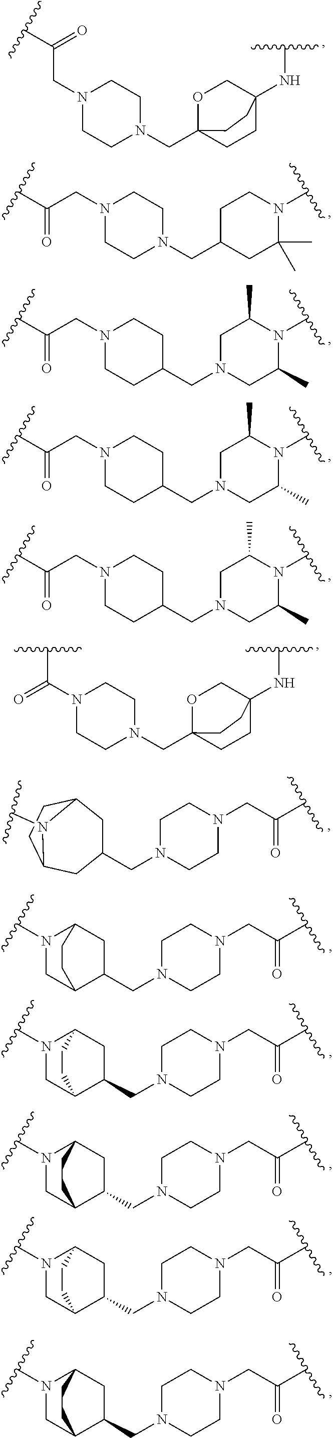

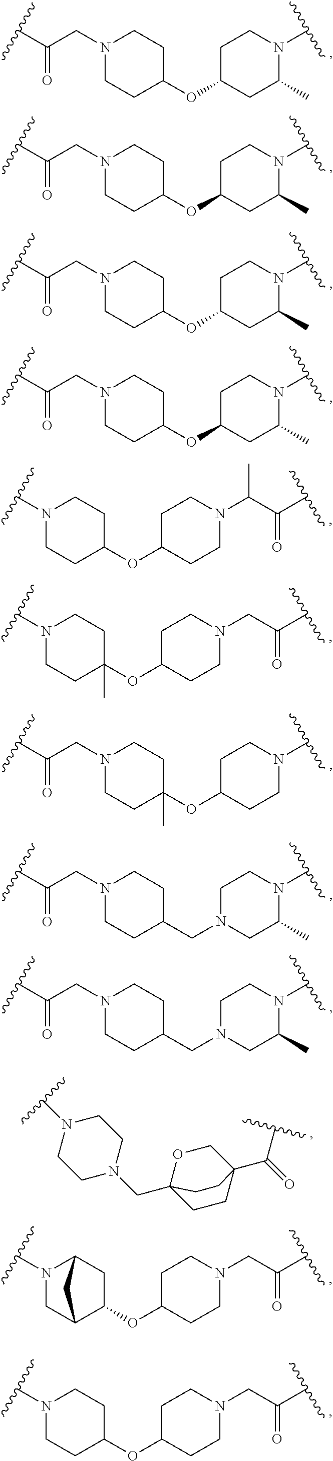

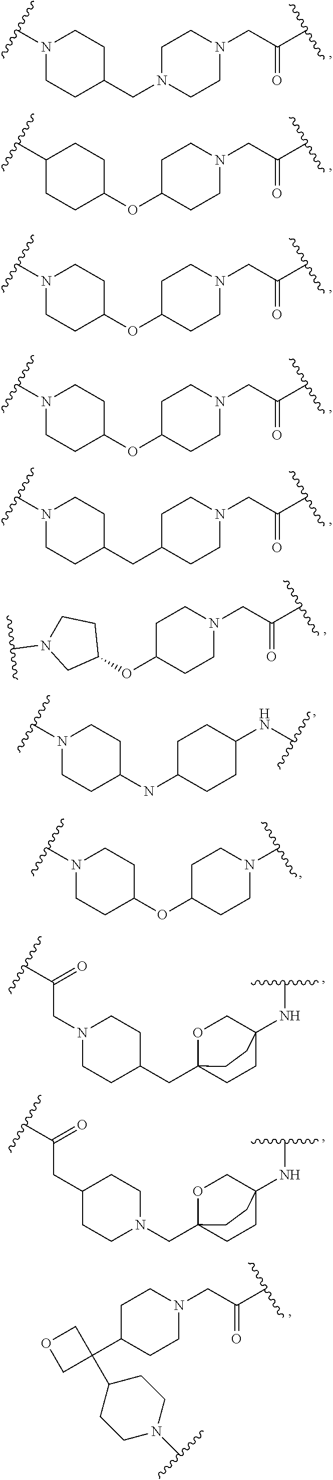

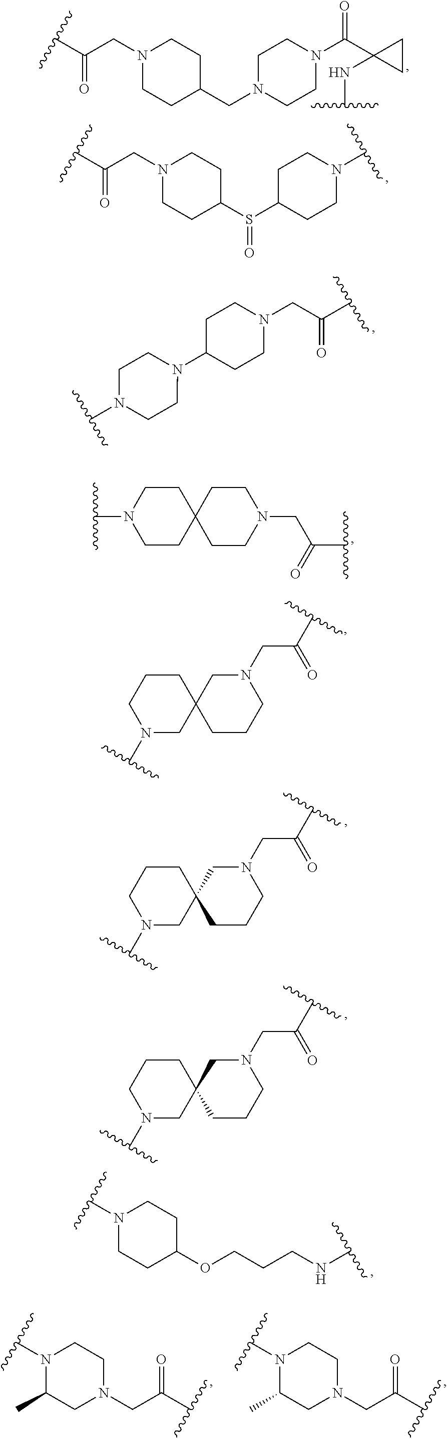

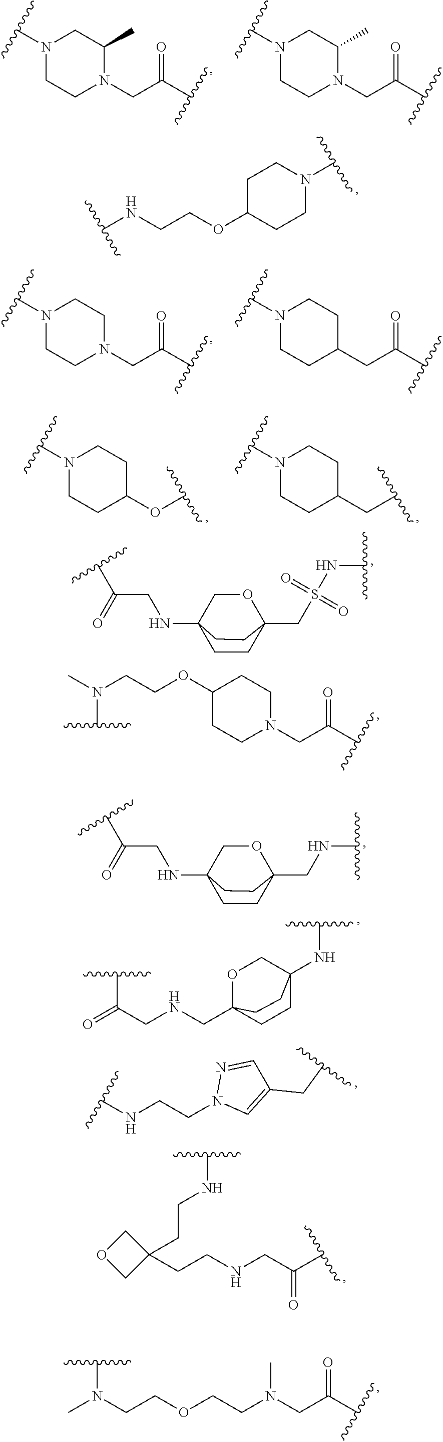

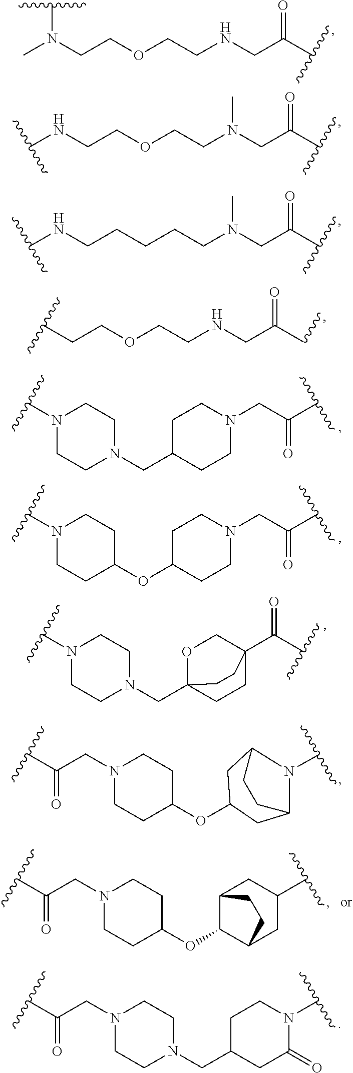

In some embodiments L is

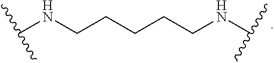 [SMILES: *NCCCCCN*]

In some embodiments, is

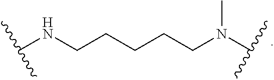 [SMILES: *NCCCCCN(*)C]

In some embodiments, L is

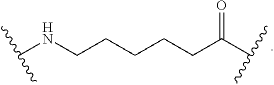 [SMILES: *NCCCCCC(*)=O]

In some embodiments, L is

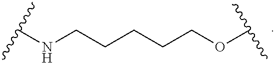 [SMILES: *NCCCCCO*]

In some embodiments, L is

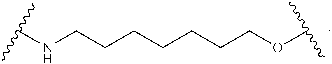 [SMILES: *NCCCCCCCO*]

In some embodiments, L is

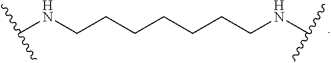 [SMILES: *NCCCCCCCN*]

In some embodiments, L is

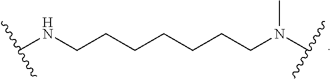 [SMILES: *NCCCCCCCN(*)C]

In some embodiments, L is

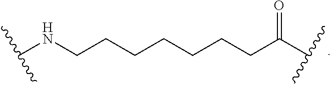 [SMILES: *NCCCCCCCC(*)=O]

In some embodiments, L is

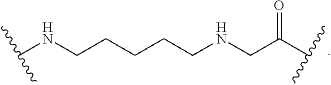 [SMILES: *NCCCCCNCC(*)=O]

In some embodiments, L is

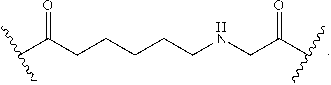 [SMILES: *C(=O)CCCCCNCC(*)=O]

In some embodiments, L is

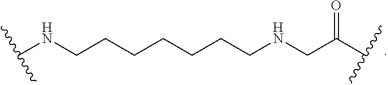 [SMILES: *NCCCCCCCNCC(*)=O]

In some embodiments, L is

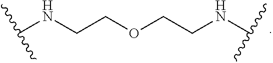 [SMILES: *NCCOCCN*]

In some embodiments, L is

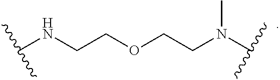 [SMILES: *NCCOCCN(*)C]

In some embodiments, L is

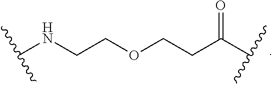 [SMILES: *NCCOCCC(*)=O]

In some embodiments, L is

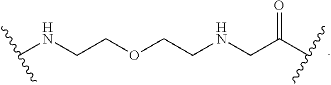 [SMILES: *NCCOCCNCC(*)=O]

In some embodiments, L is

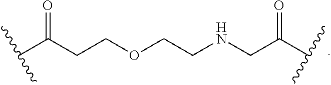 [SMILES: *C(=O)CCOCCNCC(*)=O]

In some embodiments, L is

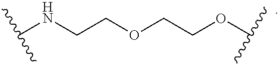 [SMILES: *NCCOCCO*]

In some embodiments, L is

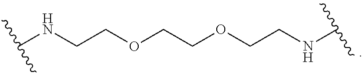 [SMILES: *NCCOCCOCCN*]

In some embodiments, L is

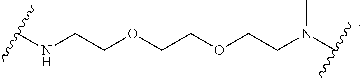 [SMILES: *NCCOCCOCCN(*)C]

In some embodiments, L is

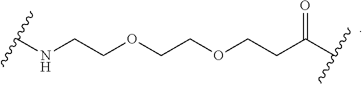 [SMILES: *NCCOCCOCCC(*)=O]

In some embodiments, L is

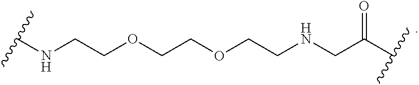 [SMILES: *NCCOCCOCCNCC(*)=O]

In some embodiments, L is

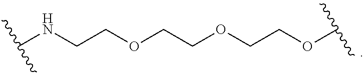 [SMILES: *NCCOCCOCCO*]

In some embodiments, L is

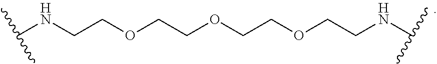 [SMILES: *NCCOCCOCCOCCN*]

In some embodiments, L is

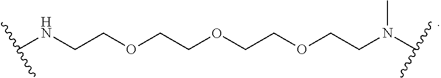 [SMILES: *NCCOCCOCCOCCN(*)C]

some embodiments, L is

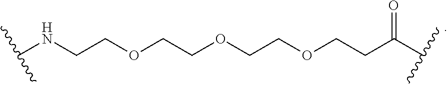 [SMILES: *NCCOCCOCCOCCC(*)=O]

In some embodiments, L is

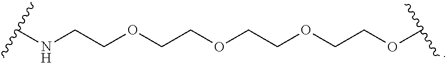 [SMILES: *NCCOCCOCCOCCO*]

In some embodiments, L is

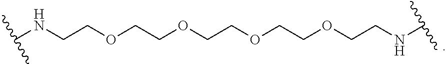 [SMILES: *NCCOCCOCCOCCOCCN*]

In some embodiments, L is

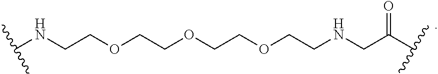 [SMILES: *NCCOCCOCCOCCNCC(*)=O]

In some embodiments, L is

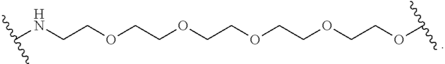 [SMILES: *NCCOCCOCCOCCOCCO*]

In some embodiments, L is

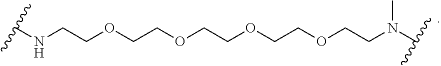 [SMILES: *NCCOCCOCCOCCOCCN(*)C]

In some embodiments, L is

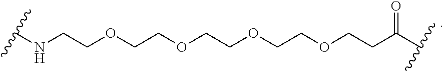 [SMILES: *NCCOCCOCCOCCOCCC(*)=O]

In some embodiments, L is

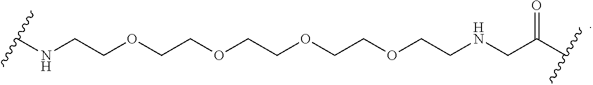 [SMILES: *NCCOCCOCCOCCOCCNCC(*)=O]

In some embodiments, L is

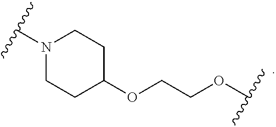 [SMILES: *OCCOC1CCN(*)CC1]

In some embodiments, L is

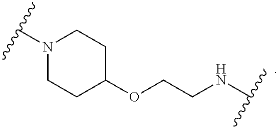 [SMILES: *NCCOC1CCN(*)CC1]

In some embodiments, L is

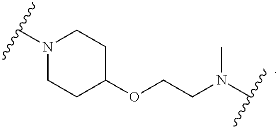 [SMILES: *N(C)CCOC1CCN(*)CC1]

In some embodiments, L is

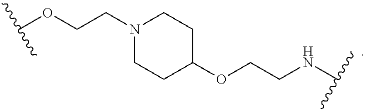 [SMILES: *NCCOC1CCN(CCO*)CC1]

In some embodiments, L is

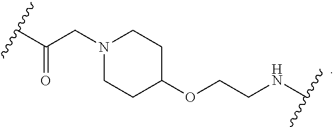 [SMILES: *NCCOC1CCN(CC(*)=O)CC1]

In some embodiments, L is

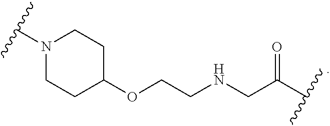 [SMILES: *C(=O)CNCCOC1CCN(*)CC1]

In some embodiments, L is

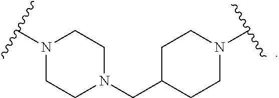 [SMILES: *N1CCC(CN2CCN(*)CC2)CC1]

In some embodiments, L is

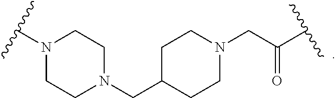 [SMILES: *C(=O)CN1CCC(CN2CCN(*)CC2)CC1]

In some embodiments, L is

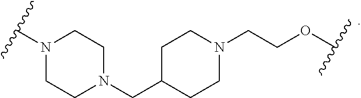 [SMILES: *OCCN1CCC(CN2CCN(*)CC2)CC1]

In some embodiments, L is

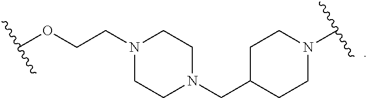 [SMILES: *OCCN1CCN(CC2CCN(*)CC2)CC1]

In some embodiments, L is

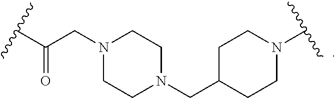 [SMILES: *C(=O)CN1CCN(CC2CCN(*)CC2)CC1]

In some embodiments, L is

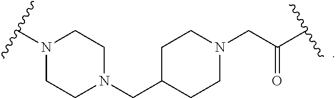 [SMILES: *C(=O)CN1CCC(CN2CCN(*)CC2)CC1]

In some embodiments, L is

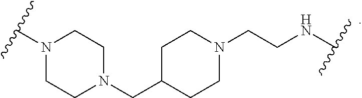 [SMILES: *NCCN1CCC(CN2CCN(*)CC2)CC1]

In some embodiments, L is

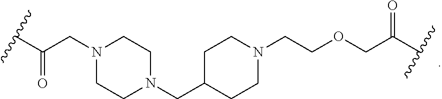 [SMILES: *C(=O)COCCN1CCC(CN2CCN(CC(*)=O)CC2)CC1]

In some embodiments, L is

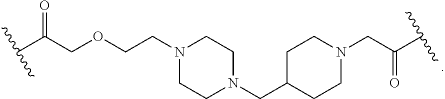 [SMILES: *C(=O)COCCN1CCN(CC2CCN(CC(*)=O)CC2)CC1]

In some embodiments, L is

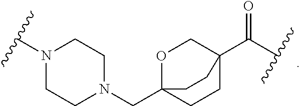 [SMILES: *C(=O)C12CCC(CN3CCN(*)CC3)(CC1)OC2]

In some embodiments, L is

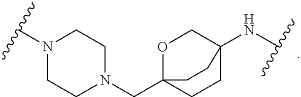 [SMILES: *NC12CCC(CN3CCN(*)CC3)(CC1)OC2]

In some embodiments, L is

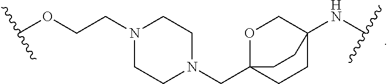 [SMILES: *NC12CCC(CN3CCN(CCO*)CC3)(CC1)OC2]

In some embodiments, L is

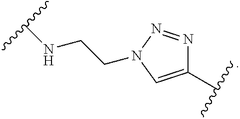 [SMILES: *NCCn1cc(*)nn1]

In some embodiments, L is

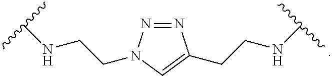 [SMILES: *NCCc1cn(CCN*)nn1]

In some embodiments, L is

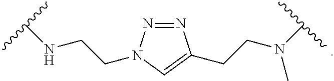 [SMILES: *NCCn1cc(CCN(*)C)nn1]

In some embodiments, L is

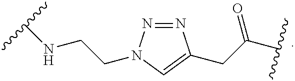 [SMILES: *NCCn1cc(CC(*)=O)nn1]

In some embodiments, L is

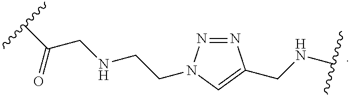 [SMILES: *NCc1cn(CCNCC(*)=O)nn1]

In some embodiments, L is

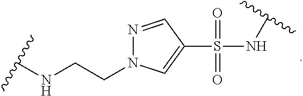 [SMILES: *NCCn1cc(S(=O)(=O)N*)cn1]

In some embodiments, L is

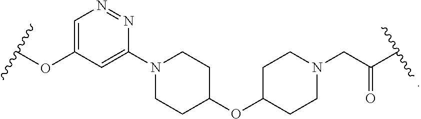 [SMILES: *Oc1cnnc(N2CCC(OC3CCN(CC(*)=O)CC3)CC2)c1]

In some embodiments, L is

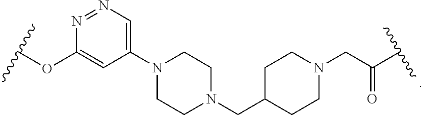 [SMILES: *Oc1cc(N2CCN(CC3CCN(CC(*)=O)CC3)CC2)cnn1]

In some embodiments, L is

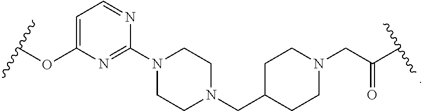 [SMILES: *Oc1ccnc(N2CCN(CC3CCN(CC(*)=O)CC3)CC2)n1]

In some embodiments, L is

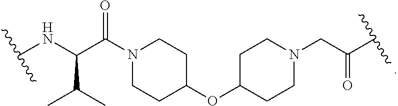 [SMILES: *N[C@@H](C(=O)N1CCC(OC2CCN(CC(*)=O)CC2)CC1)C(C)C]

In some embodiments, L is

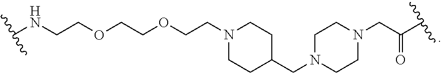 [SMILES: *NCCOCCOCCN1CCC(CN2CCN(CC(*)=O)CC2)CC1]

In some embodiments, L is

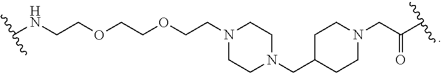 [SMILES: *NCCOCCOCCN1CCN(CC2CCN(CC(*)=O)CC2)CC1]

In some embodiments, L is

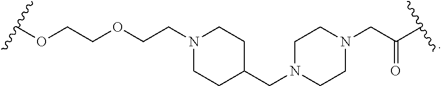 [SMILES: *OCCOCCN1CCC(CN2CCN(CC(*)=O)CC2)CC1]

In some embodiments, L is

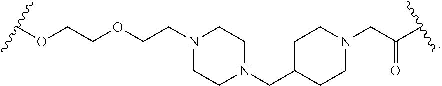 [SMILES: *OCCOCCN1CCN(CC2CCN(CC(*)=O)CC2)CC1]

In some embodiments, L is

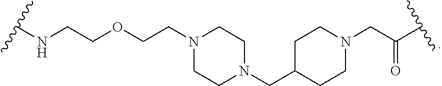 [SMILES: *NCCOCCN1CCN(CC2CCN(CC(*)=O)CC2)CC1]

In some embodiments, L is

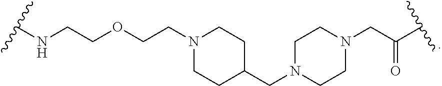 [SMILES: *NCCOCCN1CCC(CN2CCN(CC(*)=O)CC2)CC1]

In some embodiments, L is

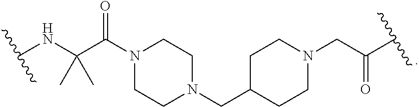 [SMILES: *NC(C)(C)C(=O)N1CCN(CC2CCN(CC(*)=O)CC2)CC1]

In some embodiments, L is

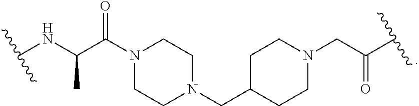 [SMILES: *N[C@H](C)C(=O)N1CCN(CC2CCN(CC(*)=O)CC2)CC1]

In some embodiments, L is

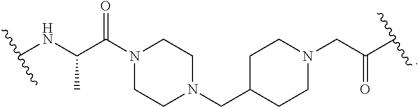 [SMILES: *N[C@@H](C)C(=O)N1CCN(CC2CCN(CC(*)=O)CC2)CC1]

In some embodiments, L is

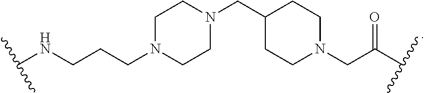 [SMILES: *NCCCN1CCN(CC2CCN(CC(*)=O)CC2)CC1]

In some embodiments, L is

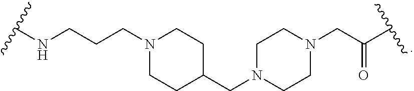 [SMILES: *NCCCN1CCC(CN2CCN(CC(*)=O)CC2)CC1]

In some embodiments, L is

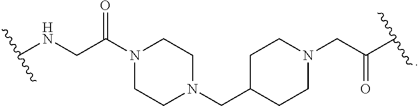 [SMILES: *NCC(=O)N1CCN(CC2CCN(CC(*)=O)CC2)CC1]

In some embodiments, L is

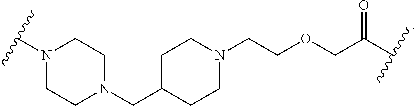 [SMILES: *C(=O)COCCN1CCC(CN2CCN(*)CC2)CC1]

In some embodiments, L is

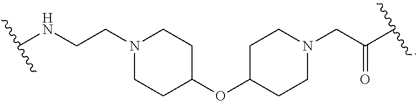 [SMILES: *NCCN1CCC(OC2CCN(CC(*)=O)CC2)CC1]

In some embodiments, L is

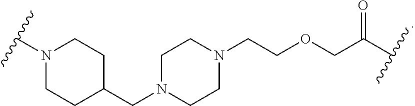 [SMILES: *C(=O)COCCN1CCN(CC2CCN(*)CC2)CC1]

In some embodiments, L is

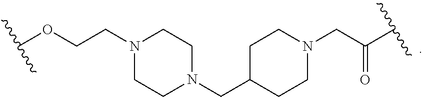 [SMILES: *OCCN1CCN(CC2CCN(CC(*)=O)CC2)CC1]

In some embodiments, L is

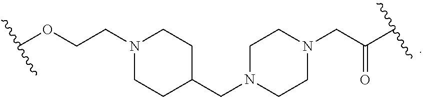 [SMILES: *OCCN1CCC(CN2CCN(CC(*)=O)CC2)CC1]

In some embodiments, L is

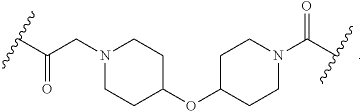 [SMILES: *C(=O)CN1CCC(OC2CCN(C(*)=O)CC2)CC1]

In some embodiments, L is

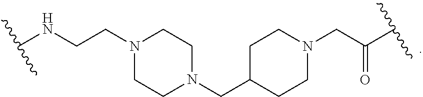 [SMILES: *NCCN1CCN(CC2CCN(CC(*)=O)CC2)CC1]

In some embodiments, L is

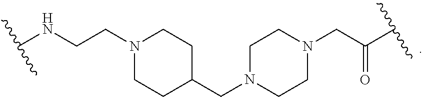 [SMILES: *NCCN1CCC(CN2CCN(CC(*)=O)CC2)CC1]

In some embodiments, L is

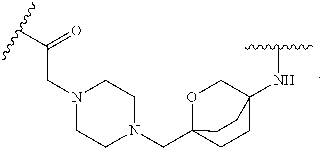 [SMILES: *NC12CCC(CN3CCN(CC(*)=O)CC3)(CC1)OC2]

In some embodiments, L is

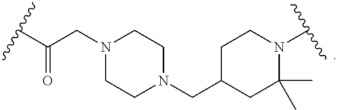 [SMILES: *C(=O)CN1CCN(CC2CCN(*)C(C)(C)C2)CC1]

In some embodiments, L is

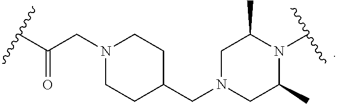 [SMILES: *C(=O)CN1CCC(CN2C[C@@H](C)N(*)[C@@H](C)C2)CC1]

In some embodiments, L is

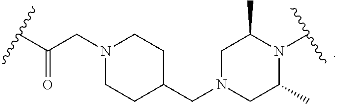 [SMILES: *C(=O)CN1CCC(CN2C[C@@H](C)N(*)[C@H](C)C2)CC1]

In some embodiments, L is

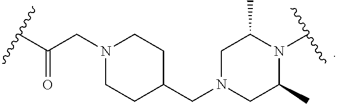 [SMILES: *C(=O)CN1CCC(CN2C[C@H](C)N(*)[C@@H](C)C2)CC1]

In some embodiments, L is

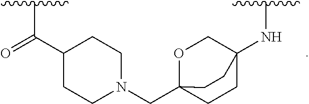 [SMILES: *NC12CCC(CN3CCC(C(*)=O)CC3)(CC1)OC2]

In some embodiments, L is

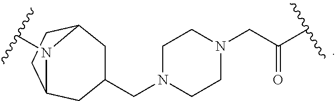 [SMILES: *C(=O)CN1CCN(CC2CC3CCC(C2)N3*)CC1]

In some embodiments, L is

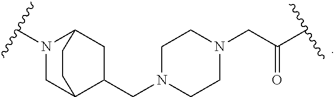 [SMILES: *C(=O)CN1CCN(CC2CC3CCC2CN3*)CC1]

In some embodiments, L is

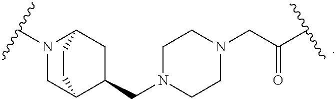 [SMILES: *C(=O)CN1CCN(C[C@@H]2C[C@H]3CC[C@@H]2CN3*)CC1]

In some embodiments, L is

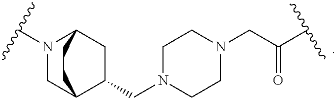 [SMILES: *C(=O)CN1CCN(C[C@H]2C[C@@H]3CC[C@H]2CN3*)CC1]

In some embodiments, L is

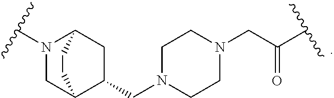 [SMILES: *C(=O)CN1CCN(C[C@H]2C[C@H]3CC[C@@H]2CN3*)CC1]

In some embodiments, L is

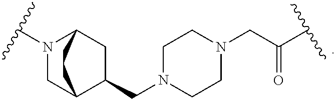 [SMILES: *C(=O)CN1CCN(C[C@@H]2C[C@@H]3CC[C@H]2CN3*)CC1]

some embodiments, L is

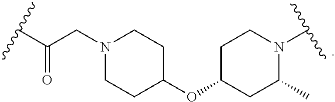 [SMILES: *C(=O)CN1CCC(O[C@@H]2CCN(*)[C@H](C)C2)CC1]

In some embodiments, L is

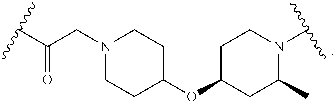 [SMILES: *C(=O)CN1CCC(O[C@H]2CCN(*)[C@@H](C)C2)CC1]

In some embodiments, is

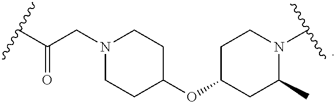 [SMILES: *C(=O)CN1CCC(O[C@@H]2CCN(*)[C@@H](C)C2)CC1]

In some embodiments, is

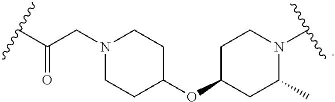 [SMILES: *C(=O)CN1CCC(O[C@H]2CCN(*)[C@H](C)C2)CC1]

In some embodiments, L is

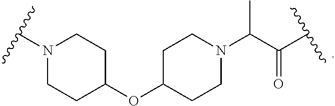 [SMILES: *C(=O)C(C)N1CCC(OC2CCN(*)CC2)CC1]

In some embodiments, L is

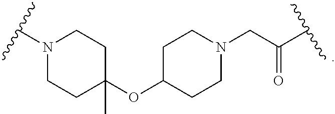 [SMILES: *C(=O)CN1CCC(OC2(C)CCN(*)CC2)CC1]

In some embodiments, L is

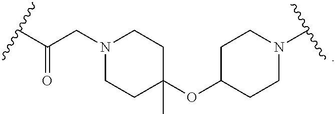 [SMILES: *C(=O)CN1CCC(C)(OC2CCN(*)CC2)CC1]

In some embodiments, L is

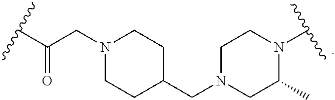 [SMILES: *C(=O)CN1CCC(CN2CCN(*)[C@H](C)C2)CC1]

In some embodiments, L is

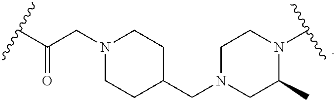 [SMILES: *C(=O)CN1CCC(CN2CCN(*)[C@@H](C)C2)CC1]

In some embodiments, L is

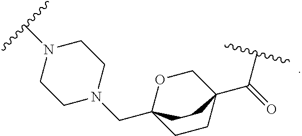 [SMILES: *C(=O)[C@]12CC[C@](CN3CCN(*)CC3)(CC1)OC2]

In some embodiments, L is

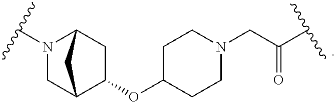 [SMILES: *C(=O)CN1CCC(O[C@H]2C[C@@H]3C[C@H]2CN3*)CC1]

In some embodiments, L is

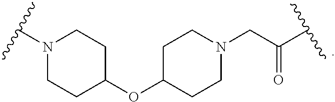 [SMILES: *C(=O)CN1CCC(OC2CCN(*)CC2)CC1]

In some embodiments, L is

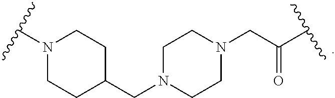 [SMILES: *C(=O)CN1CCN(CC2CCN(*)CC2)CC1]

In some embodiments, L is

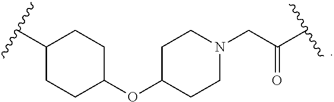 [SMILES: *C(=O)CN1CCC(OC2CCC(*)CC2)CC1]

In some embodiments, L is

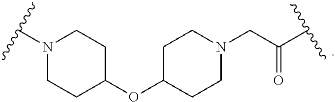 [SMILES: *C(=O)CN1CCC(OC2CCN(*)CC2)CC1]

In some embodiments, L is

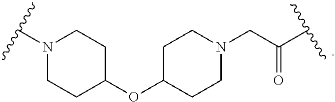 [SMILES: *C(=O)CN1CCC(OC2CCN(*)CC2)CC1]

In some embodiments, L is

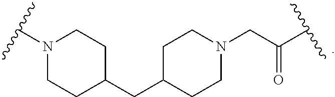 [SMILES: *C(=O)CN1CCC(CC2CCN(*)CC2)CC1]

In some embodiments, L is

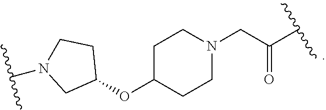 [SMILES: *C(=O)CN1CCC(O[C@H]2CCN(*)C2)CC1]

In some embodiments, L is

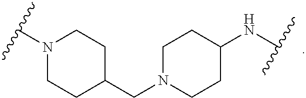 [SMILES: *NC1CCN(CC2CCN(*)CC2)CC1]

In some embodiments, L is

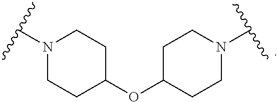 [SMILES: *N1CCC(OC2CCN(*)CC2)CC1]

In some embodiments, L is

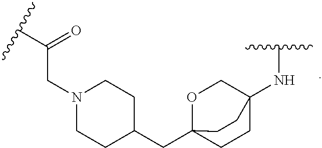 [SMILES: *NC12CCC(CC3CCN(CC(*)=O)CC3)(CC1)OC2]

some embodiments, L is

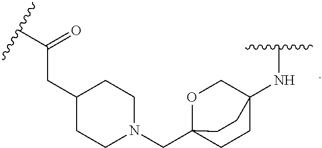 [SMILES: *NC12CCC(CN3CCC(CC(*)=O)CC3)(CC1)OC2]

In some embodiments, L is

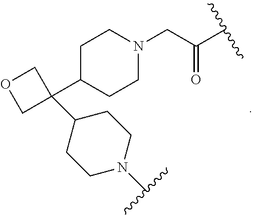 [SMILES: *C(=O)CN1CCC(C2(C3CCN(*)CC3)COC2)CC1]

In some embodiments, is

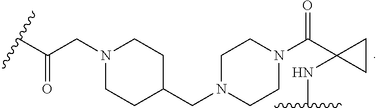 [SMILES: *NC1(C(=O)N2CCN(CC3CCN(CC(*)=O)CC3)CC2)CC1]

In some embodiments, is

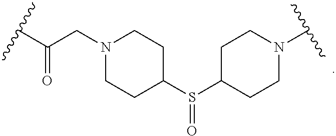 [SMILES: *C(=O)CN1CCC(S(=O)C2CCN(*)CC2)CC1]

In some embodiments, L is

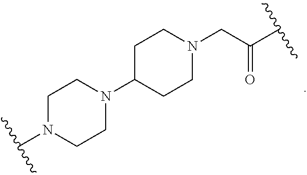 [SMILES: *C(=O)CN1CCC(N2CCN(*)CC2)CC1]

In some embodiments, L is

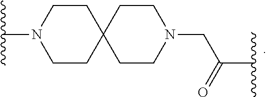 [SMILES: *C(=O)CN1CCC2(CCN(*)CC2)CC1]

In some embodiments, L is

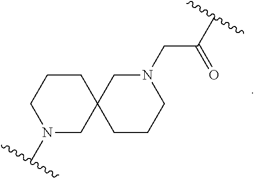 [SMILES: *C(=O)CN1CCCC2(CCCN(*)C2)C1]

In some embodiments, L is

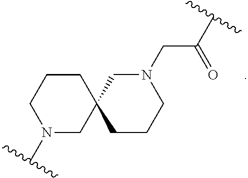 [SMILES: *C(=O)CN1CCC[C@@]2(CCCN(*)C2)C1]

In some embodiments, L is

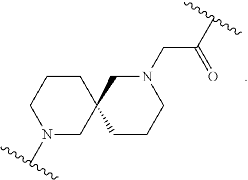 [SMILES: *C(=O)CN1CCC[C@]2(CCCN(*)C2)C1]

In some embodiments, L is

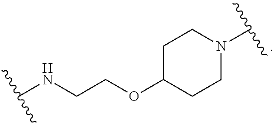 [SMILES: *NCCOC1CCN(*)CC1]

In some embodiments, L is

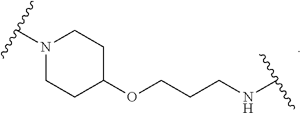 [SMILES: *NCCCOC1CCN(*)CC1]

In some embodiments, L is

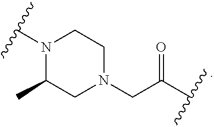 [SMILES: *C(=O)CN1CCN(*)[C@H](C)C1]

In some embodiments, L is

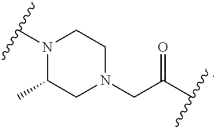 [SMILES: *C(=O)CN1CCN(*)[C@@H](C)C1]

In some embodiments, L is

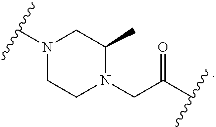 [SMILES: *C(=O)CN1CCN(*)C[C@H]1C]

In some embodiments, L is

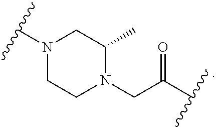 [SMILES: *C(=O)CN1CCN(*)C[C@@H]1C]

In some embodiments, L is

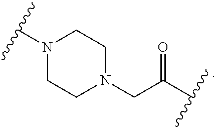 [SMILES: *C(=O)CN1CCN(*)CC1]

In some embodiments, L is

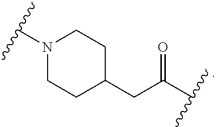 [SMILES: *C(=O)CC1CCN(*)CC1]

In some embodiments, L is

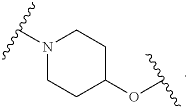 [SMILES: *OC1CCN(*)CC1]

In some embodiments, L is

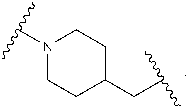 [SMILES: *CC1CCN(*)CC1]

In some embodiments, L is

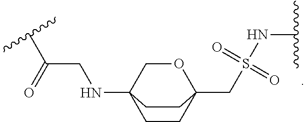 [SMILES: *NS(=O)(=O)CC12CCC(NCC(*)=O)(CC1)CO2]

In some embodiments, L is

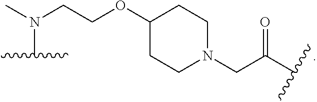 [SMILES: *C(=O)CN1CCC(OCCN(*)C)CC1]

some embodiments, L is

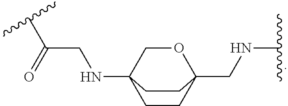 [SMILES: *NCC12CCC(NCC(*)=O)(CC1)CO2]

In some embodiments, L is

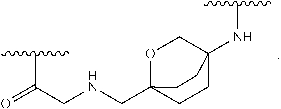 [SMILES: *NC12CCC(CNCC(*)=O)(CC1)OC2]

In some embodiments, L is

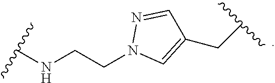 [SMILES: *Cc1cnn(CCN*)c1]

In some embodiments, L is

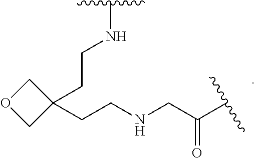 [SMILES: *NCCC1(CCNCC(*)=O)COC1]

In some embodiments, L is

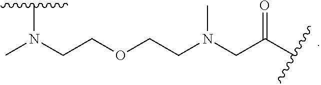 [SMILES: *C(=O)CN(C)CCOCCN(*)C]

In some embodiments, L is

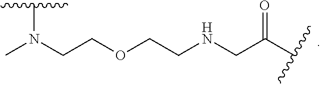 [SMILES: *C(=O)CNCCOCCN(*)C]

In some embodiments, L is

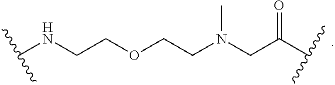 [SMILES: *NCCOCCN(C)CC(*)=O]

In some embodiments, L is

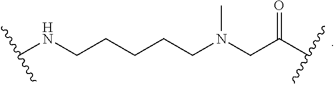 [SMILES: *NCCCCCN(C)CC(*)=O]

In some embodiments, L is

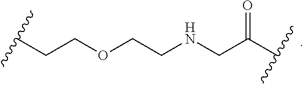 [SMILES: *CCOCCNCC(*)=O]

In some embodiments, L is

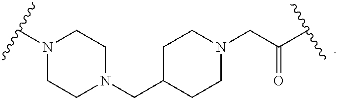 [SMILES: *C(=O)CN1CCC(CN2CCN(*)CC2)CC1]

In some embodiments, L is

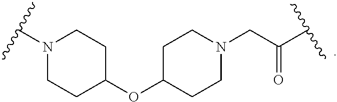 [SMILES: *C(=O)CN1CCC(OC2CCN(*)CC2)CC1]

In some embodiments, L is

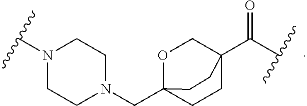 [SMILES: *C(=O)C12CCC(CN3CCN(*)CC3)(CC1)OC2]

In some embodiments, L is

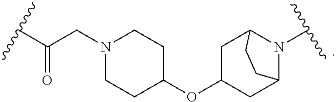 [SMILES: *C(=O)CN1CCC(OC2CC3CCC(C2)N3*)CC1]

In some embodiments, L is

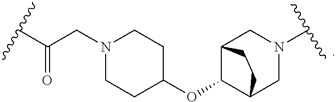 [SMILES: *C(=O)CN1CCC(O[C@H]2[C@@H]3CC[C@H]2CN(*)C3)CC1]

In some embodiments, L is

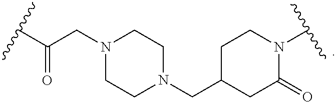 [SMILES: *C(=O)CN1CCN(CC2CCN(*)C(=O)C2)CC1]

In some embodiments, L is selected from those depicted in Table 1, below.

In some embodiments, L is selected from those depicted in Table B, below.

In some embodiments, the present invention provides the compound of formula I-a, wherein DBM is

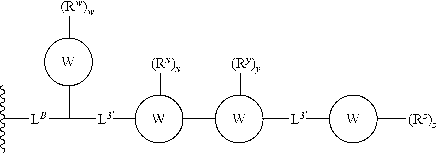

from formula I-bb or I-bb′, to provide a compound of formula I-abb:

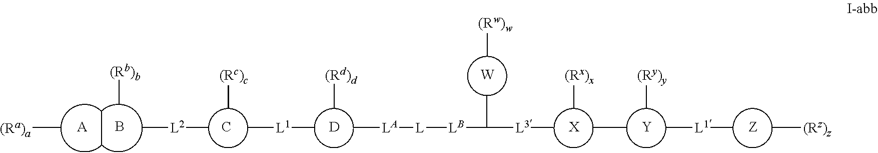

or a pharmaceutically acceptable salt thereof, wherein each of ring A, ring B, ring C, ring, D, L¹, L², L^{A}, R^{a}, R^{b}, R^{c}, R^{d}, a, b, c, d, L, ring W, ring X, ring Y, ring Z, L^{B}, L^{3′}, L^{1′}, R^{w}, R^{x}, R^{y}, R^{z}, w, x, y, z, is independently as defined above and described in embodiments herein, both singly and in combination.

In some embodiments, the present invention provides the compound of formula I-a, wherein DBM is

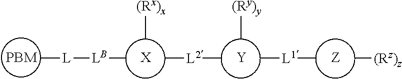

from formula I-cc, to provide a compound of formula I-acc:

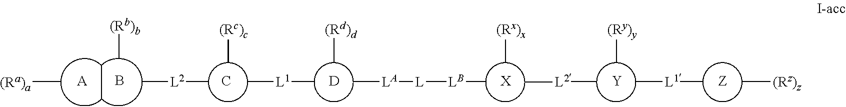

or a pharmaceutically acceptable salt thereof, wherein each of ring A, ring B, ring C, ring, D, L¹, L², L^{A}, R^{a}, R^{b}, R^{c}, R^{d}, a, b, c, d, L, ring X, ring Y, ring Z, L^{B}, L^{2′}, L^{1′}, R^{x}, R^{y}, R^{z}, x, y, z, is independently as defined above and described in embodiments herein, both singly and in combination.

In some embodiments, the present invention provides the compound of formula I-a, wherein DBM is

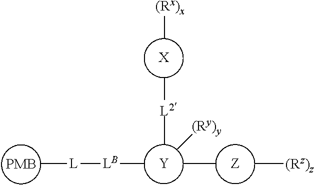

from formula I-ff or I-ff′, to provide a compound of formula I-aff:

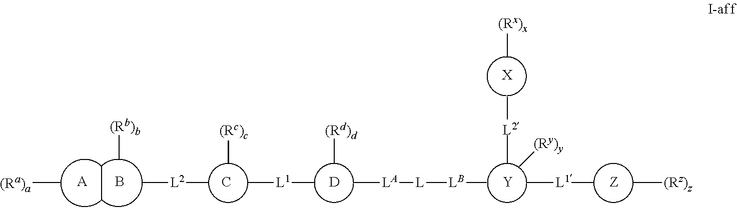

or a pharmaceutically acceptable salt thereof, wherein each of ring A, ring B, ring C, ring, D, L¹, L², L^{A}, R^{a}, R^{b}, R^{c}, R^{d}, a, b, c, d, L, ring X, ring Y, ring Z, L^{B}, L^{2′}, R^{x}, R^{y}, R^{z}, x, y, z, is independently as defined above and described in embodiments herein, both singly and in combination.

In some embodiments, the present invention provides the compound of formula I-a, wherein DBM is

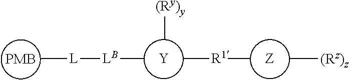

from formula I-gg, to provide a compound of formula I-agg:

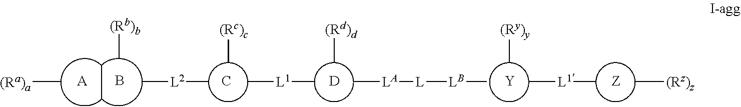

or a pharmaceutically acceptable salt thereof, wherein each of ring A, ring B, ring C, ring, D, L¹, L², L^{A}, R^{a}, R^{b}, R^{c}, R^{d}, a, b, c, d, L, ring Y, ring Z, L^{B}, L^{1′}, R^{y}, R^{z}, y, z, is independently as defined above and described in embodiments herein, both singly and in combination.

In some embodiments, the present invention provides the compound of formula I-a, wherein DBM is

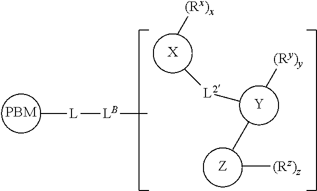

from formula I-hh, to provide a compound of formula I-ahh:

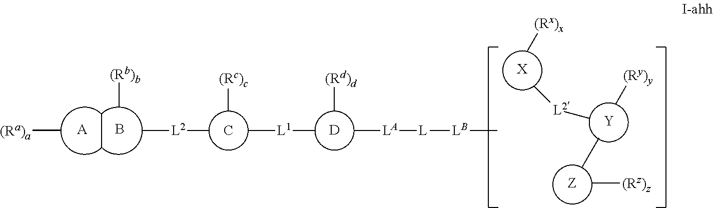

or a pharmaceutically acceptable salt thereof, wherein each of ring A, ring B, ring C, ring, D, L¹, L², L^{A}, R^{a}, R^{b}, R^{c}, R^{d}, a, b, c, d, L, ring X, ring Y, ring Z, L^{B}, L^{2′}, R^{x}, R^{y}, R^{z}, x, y, z, is independently as defined above and described in embodiments herein, both singly and in combination.

In some embodiments, the present invention provides the compound of formula I-a, wherein DBM is

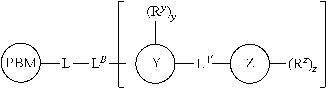

from formula I-ii, to provide a compound of formula I-aii:

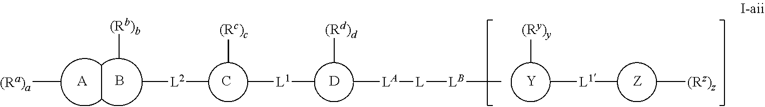

or a pharmaceutically acceptable salt thereof, wherein each of ring A, ring B, ring C, ring, D, L¹, L², L^{A}, R^{a}, R^{b}, R^{c}, R^{d}, a, b, c, d, L, ring Y, ring Z, L^{B}, L^{1′}, R^{y}, R^{z}, y, z, is independently as defined above and described in embodiments herein, both singly and in combination.

In some embodiments, the present invention provides the compound of formula I-a, wherein DBM is

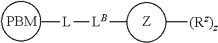

from formula I-kk, to provide a compound of formula I-akk:

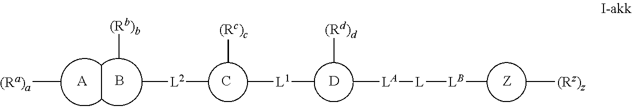

or a pharmaceutically acceptable salt thereof, wherein each of ring A, ring B, ring C, ring, D, L¹, L², L^{A}, R^{a}, R^{b}, R^{c}, R^{d}, a, b, c, d, L, ring Z, L^{B}, R^{z}, z, is independently as defined above and described in embodiments herein, both singly and in combination.

In some embodiments, the present invention provides the compound of formula I-a, wherein DBM is

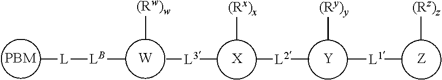

from formula I-ll, to provide a compound of formula I-all:

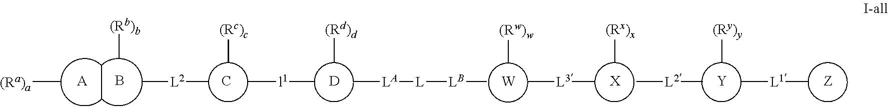

or a pharmaceutically acceptable salt thereof, wherein each of ring A, ring B, ring C, ring, D, L¹, L², L^{A}, R^{a}, R^{b}, R^{c}, R^{d}, a, b, c, d, L, ring W, ring X, ring Y, ring Z, L^{B}, L^{3′}, L^{2′}, L^{1′}, R^{w}, R^{x}, R^{y}, R^{z}, w, x, y, z, is independently as defined above and described in embodiments herein, both singly and in combination.

TABLE A
Exemplified Deubiquitinase binding moiety (DBM)

| | |
|---|---|
| 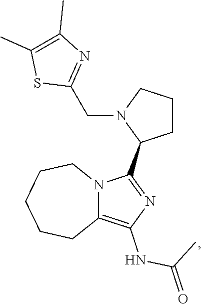 [SMILES: CC(=O)Nc1nc([C@@H]2CCCN2Cc2nc(C)c(C)s2)n2c1CCCCC2] | (a) |
| 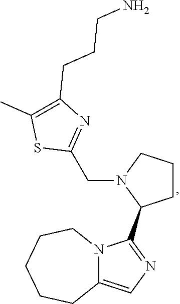 [SMILES: Cc1sc(CN2CCC[C@H]2c2ncc3n2CCCCC3)nc1CCCN] | (b) |
| 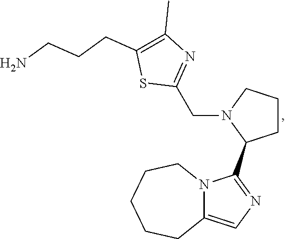 [SMILES: Cc1nc(CN2CCC[C@H]2c2ncc3n2CCCCC3)sc1CCCN] | (c) |
| 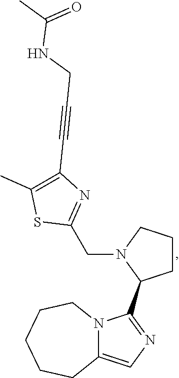 [SMILES: CC(=O)NCC#Cc1nc(CN2CCC[C@H]2c2ncc3n2CCCCC3)sc1C] | (d) |
| 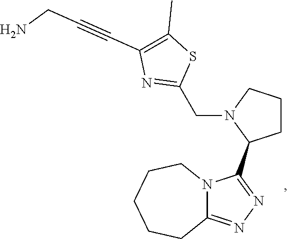 [SMILES: Cc1sc(CN2CCC[C@H]2c2nnc3n2CCCCC3)nc1C#CCN] | (e) |
| 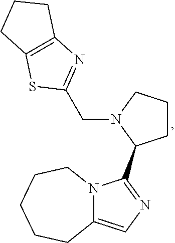 [SMILES: c1nc([C@@H]2CCCN2Cc2nc3c(s2)CCC3)n2c1CCCCC2] | (f) |
| 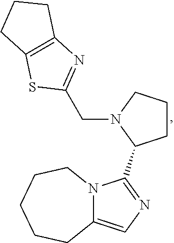 [SMILES: c1nc([C@H]2CCCN2Cc2nc3c(s2)CCC3)n2c1CCCCC2] | (g) |
| 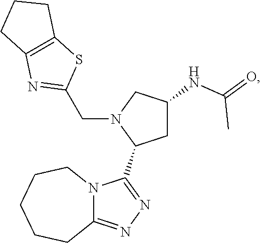 [SMILES: CC(=O)N[C@@H]1C[C@H](c2nnc3n2CCCCC3)N(Cc2nc3c(s2)CCC3)C1] | (h) |
| 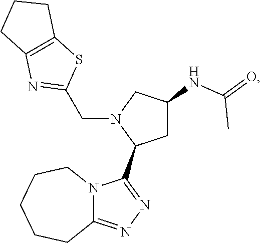 [SMILES: CC(=O)N[C@H]1C[C@@H](c2nnc3n2CCCCC3)N(Cc2nc3c(s2)CCC3)C1] | (i) |
| 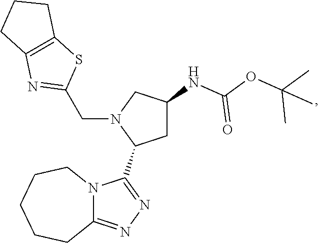 [SMILES: CC(C)(C)OC(=O)N[C@H]1C[C@H](c2nnc3n2CCCCC3)N(Cc2nc3c(s2)CCC3)C1] | (j) |
| 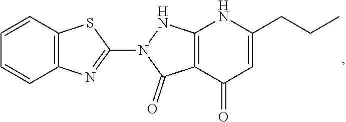 [SMILES: CCCc1cc(=O)c2c(=O)n(-c3nc4ccccc4s3)[nH]c2[nH]1] | (n) |
| 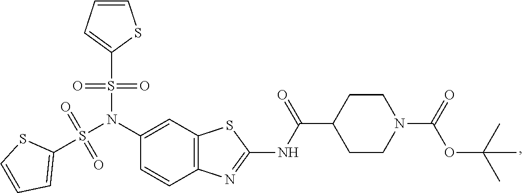 [SMILES: CC(C)(C)OC(=O)N1CCC(C(=O)Nc2nc3ccc(N(S(=O)(=O)c4cccs4)S(=O)(=O)c4cccs4)cc3s2)CC1] | (o) |
| 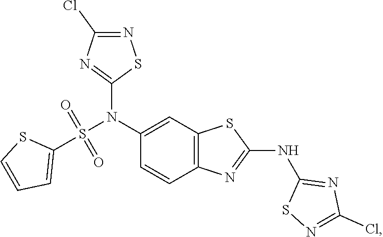 | (p) |
| 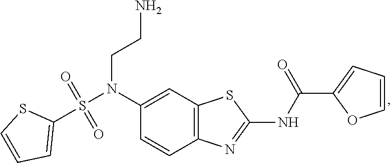 [SMILES: NCCN(c1ccc2nc(NC(=O)c3ccco3)sc2c1)S(=O)(=O)c1cccs1] | (q) |
| 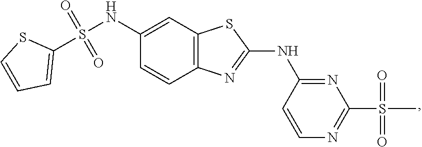 [SMILES: CS(=O)(=O)c1nccc(Nc2nc3ccc(NS(=O)(=O)c4cccs4)cc3s2)n1] | (r) |
| 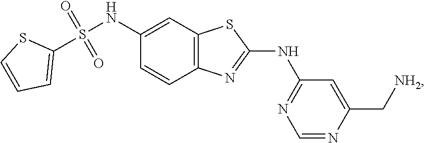 [SMILES: NCc1cc(Nc2nc3ccc(NS(=O)(=O)c4cccs4)cc3s2)ncn1] | (s) |
| 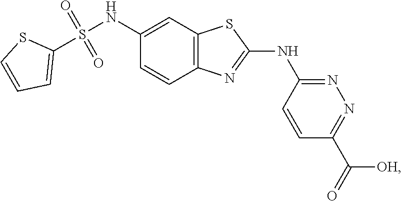 [SMILES: O=C(O)c1ccc(Nc2nc3ccc(NS(=O)(=O)c4cccs4)cc3s2)nn1] | (t) |
| 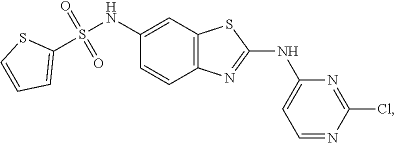 [SMILES: O=S(=O)(Nc1ccc2nc(Nc3ccnc(Cl)n3)sc2c1)c1cccs1] | (u) |
| 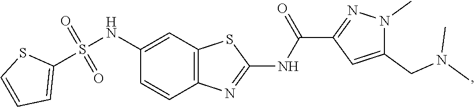 [SMILES: CN(C)Cc1cc(C(=O)Nc2nc3ccc(NS(=O)(=O)c4cccs4)cc3s2)nn1C] | (v) |
| 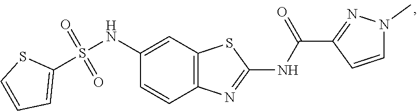 [SMILES: Cn1ccc(C(=O)Nc2nc3ccc(NS(=O)(=O)c4cccs4)cc3s2)n1] | (w) |
| 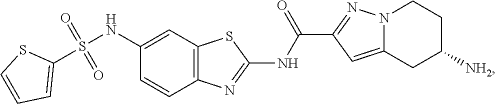 [SMILES: N[C@H]1CCn2nc(C(=O)Nc3nc4ccc(NS(=O)(=O)c5cccs5)cc4s3)cc2C1] | (x) |
| 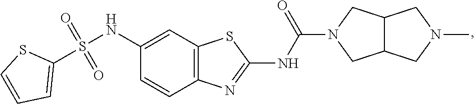 [SMILES: CN1CC2CN(C(=O)Nc3nc4ccc(NS(=O)(=O)c5cccs5)cc4s3)CC2C1] | (y) |
| 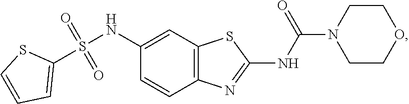 [SMILES: O=C(Nc1nc2ccc(NS(=O)(=O)c3cccs3)cc2s1)N1CCOCC1] | (z) |
| 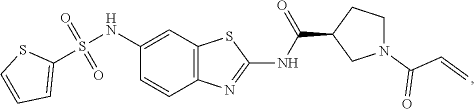 [SMILES: C=CC(=O)N1CC[C@H](C(=O)Nc2nc3ccc(NS(=O)(=O)c4cccs4)cc3s2)C1] | (aa) |
| 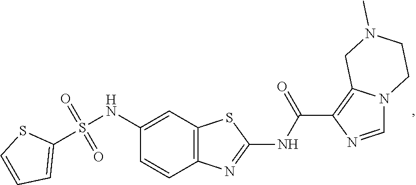 [SMILES: CN1CCn2cnc(C(=O)Nc3nc4ccc(NS(=O)(=O)c5cccs5)cc4s3)c2C1] | (ab) |
| 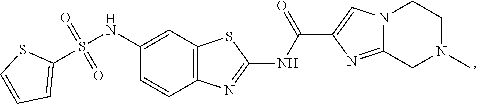 [SMILES: CN1CCn2cc(C(=O)Nc3nc4ccc(NS(=O)(=O)c5cccs5)cc4s3)nc2C1] | (ac) |
| 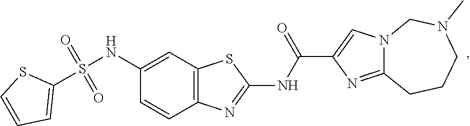 [SMILES: CN1CCCc2nc(C(=O)Nc3nc4ccc(NS(=O)(=O)c5cccs5)cc4s3)cn2C1] | (ad) |
| 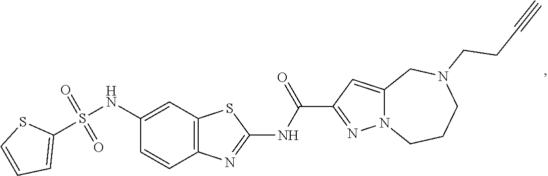 [SMILES: C#CCCN1CCCn2nc(C(=O)Nc3nc4ccc(NS(=O)(=O)c5cccs5)cc4s3)cc2C1] | (ae) |
| 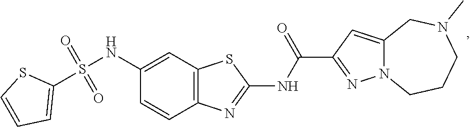 [SMILES: CN1CCCn2nc(C(=O)Nc3nc4ccc(NS(=O)(=O)c5cccs5)cc4s3)cc2C1] | (af) |
| 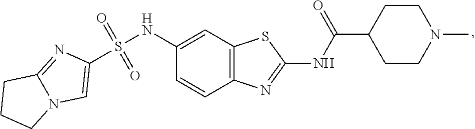 [SMILES: CN1CCC(C(=O)Nc2nc3ccc(NS(=O)(=O)c4cn5c(n4)CCC5)cc3s2)CC1] | (ag) |
| 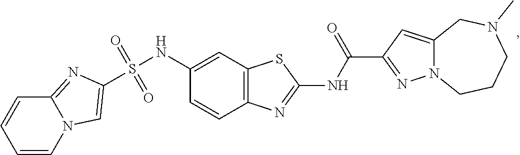 [SMILES: CN1CCCn2nc(C(=O)Nc3nc4ccc(NS(=O)(=O)c5cn6ccccc6n5)cc4s3)cc2C1] | (ah) |
| 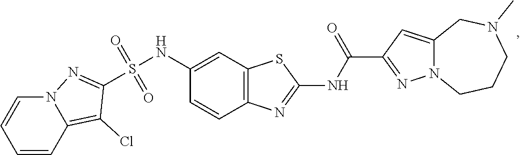 [SMILES: CN1CCCn2nc(C(=O)Nc3nc4ccc(NS(=O)(=O)c5nn6ccccc6c5Cl)cc4s3)cc2C1] | (ai) |
| 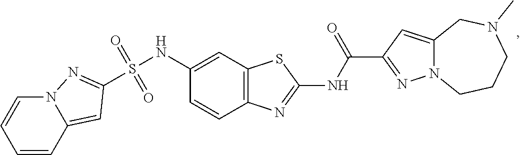 [SMILES: CN1CCCn2nc(C(=O)Nc3nc4ccc(NS(=O)(=O)c5cc6ccccn6n5)cc4s3)cc2C1] | (aj) |
| 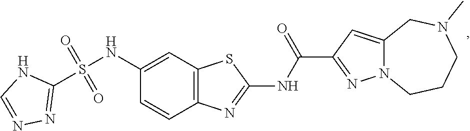 [SMILES: CN1CCCn2nc(C(=O)Nc3nc4ccc(NS(=O)(=O)c5nnc[nH]5)cc4s3)cc2C1] | (ak) |
| 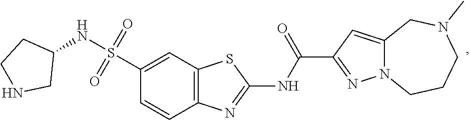 [SMILES: CN1CCCn2nc(C(=O)Nc3nc4ccc(S(=O)(=O)N[C@H]5CCNC5)cc4s3)cc2C1] | (al) |
| 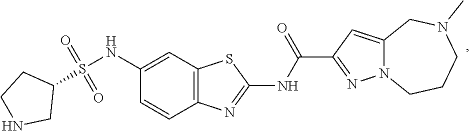 [SMILES: CN1CCCn2nc(C(=O)Nc3nc4ccc(NS(=O)(=O)[C@H]5CCNC5)cc4s3)cc2C1] | (am) |
| 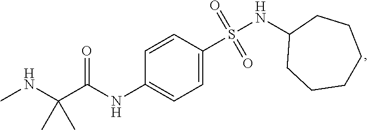 [SMILES: CNC(C)(C)C(=O)Nc1ccc(S(=O)(=O)NC2CCCCCC2)cc1] | (an) |
| 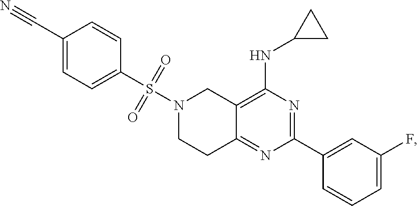 [SMILES: N#Cc1ccc(S(=O)(=O)N2CCc3nc(-c4cccc(F)c4)nc(NC4CC4)c3C2)cc1] | (ao) |
| 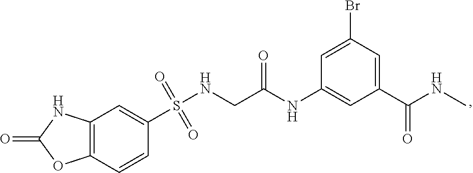 [SMILES: CNC(=O)c1cc(Br)cc(NC(=O)CNS(=O)(=O)c2ccc3oc(=O)[nH]c3c2)c1] | (ap) |
| 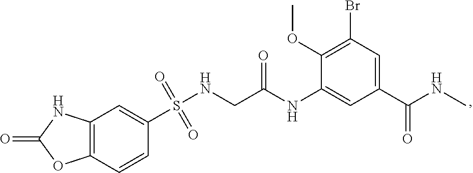 [SMILES: CNC(=O)c1cc(Br)c(OC)c(NC(=O)CNS(=O)(=O)c2ccc3oc(=O)[nH]c3c2)c1] | (aq) |
| 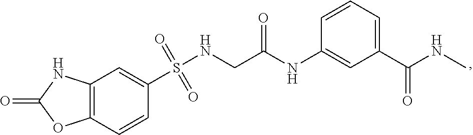 [SMILES: CNC(=O)c1cccc(NC(=O)CNS(=O)(=O)c2ccc3oc(=O)[nH]c3c2)c1] | (ar) |
| 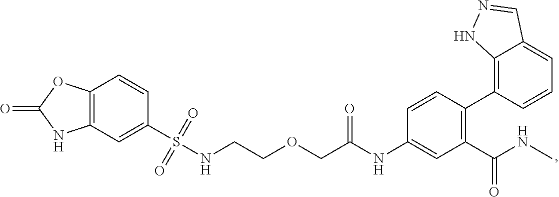 [SMILES: CNC(=O)c1cc(NC(=O)COCCNS(=O)(=O)c2ccc3oc(=O)[nH]c3c2)ccc1-c1cccc2cn[nH]c12] | (as) |
| 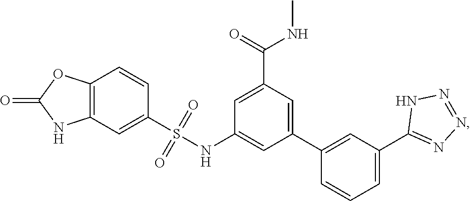 [SMILES: CNC(=O)c1cc(NS(=O)(=O)c2ccc3oc(=O)[nH]c3c2)cc(-c2cccc(-c3nnn[nH]3)c2)c1] | (at) |
| 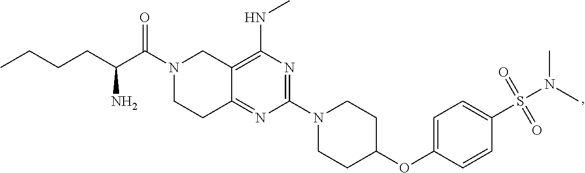 [SMILES: CCCC[C@H](N)C(=O)N1CCc2nc(N3CCC(Oc4ccc(S(=O)(=O)N(C)C)cc4)CC3)nc(NC)c2C1] | (au) |
| 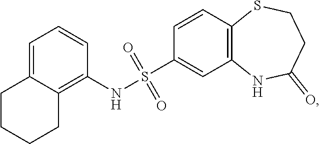 [SMILES: O=C1CCSc2ccc(S(=O)(=O)Nc3cccc4c3CCCC4)cc2N1] | (av) |
| 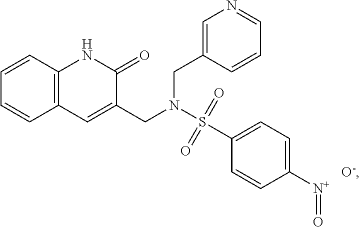 | (aw) |
| 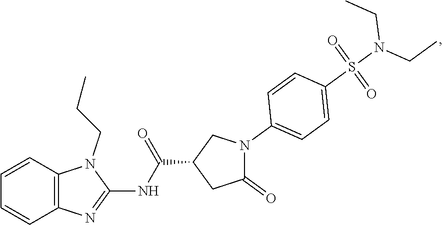 [SMILES: CCCn1c(NC(=O)[C@H]2CC(=O)N(c3ccc(S(=O)(=O)N(CC)CC)cc3)C2)nc2ccccc21] | (ax) |
| 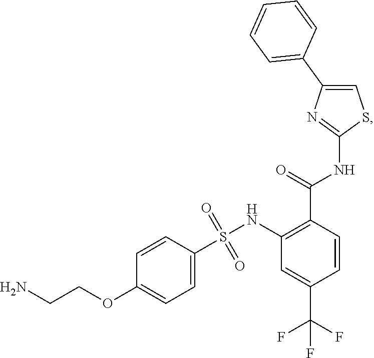 [SMILES: NCCOc1ccc(S(=O)(=O)Nc2cc(C(F)(F)F)ccc2C(=O)Nc2nc(-c3ccccc3)cs2)cc1] | (ay) |
| 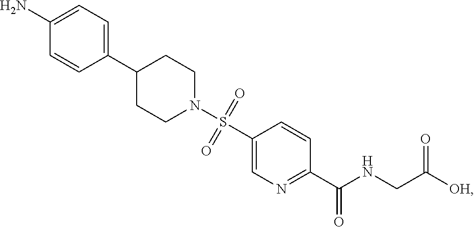 [SMILES: Nc1ccc(C2CCN(S(=O)(=O)c3ccc(C(=O)NCC(=O)O)nc3)CC2)cc1] | (az) |
| 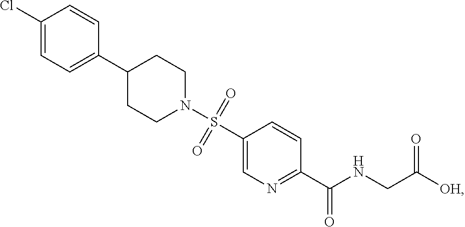 [SMILES: O=C(O)CNC(=O)c1ccc(S(=O)(=O)N2CCC(c3ccc(Cl)cc3)CC2)cn1] | (ba) |
| 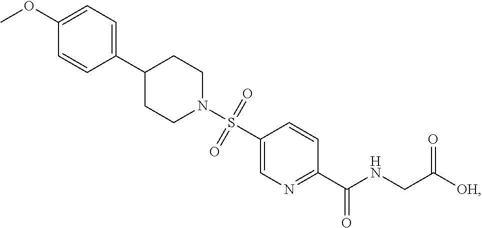 [SMILES: COc1ccc(C2CCN(S(=O)(=O)c3ccc(C(=O)NCC(=O)O)nc3)CC2)cc1] | (bb) |
| 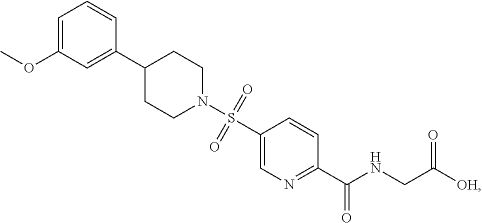 [SMILES: COc1cccc(C2CCN(S(=O)(=O)c3ccc(C(=O)NCC(=O)O)nc3)CC2)c1] | (bc) |
| 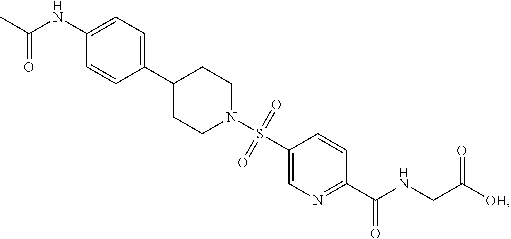 [SMILES: CC(=O)Nc1ccc(C2CCN(S(=O)(=O)c3ccc(C(=O)NCC(=O)O)nc3)CC2)cc1] | (bd) |
| 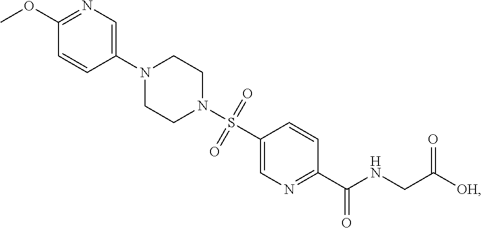 [SMILES: COc1ccc(N2CCN(S(=O)(=O)c3ccc(C(=O)NCC(=O)O)nc3)CC2)cn1] | (be) |
| 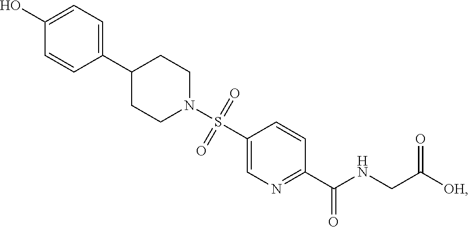 [SMILES: O=C(O)CNC(=O)c1ccc(S(=O)(=O)N2CCC(c3ccc(O)cc3)CC2)cn1] | (bf) |
| 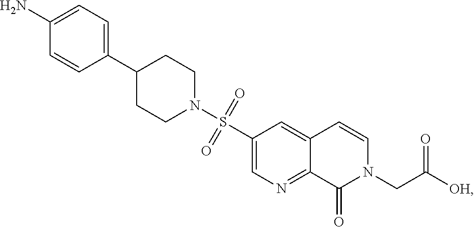 [SMILES: Nc1ccc(C2CCN(S(=O)(=O)c3cnc4c(=O)n(CC(=O)O)ccc4c3)CC2)cc1] | (bg) |
| 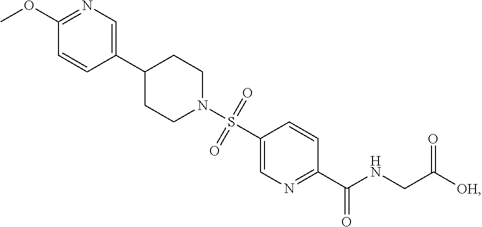 [SMILES: COc1ccc(C2CCN(S(=O)(=O)c3ccc(C(=O)NCC(=O)O)nc3)CC2)cn1] | (bh) |
| 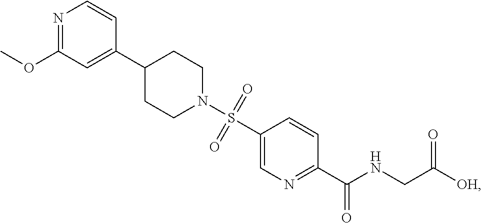 [SMILES: COc1cc(C2CCN(S(=O)(=O)c3ccc(C(=O)NCC(=O)O)nc3)CC2)ccn1] | (bi) |
| 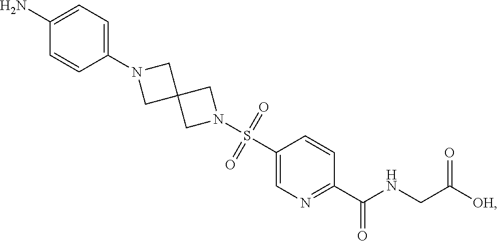 [SMILES: Nc1ccc(N2CC3(C2)CN(S(=O)(=O)c2ccc(C(=O)NCC(=O)O)nc2)C3)cc1] | (bj) |
| 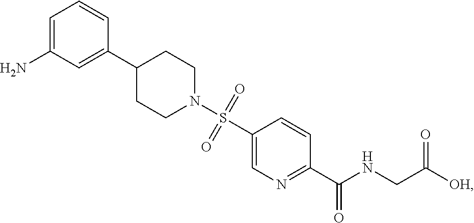 [SMILES: Nc1cccc(C2CCN(S(=O)(=O)c3ccc(C(=O)NCC(=O)O)nc3)CC2)c1] | (bk) |
| 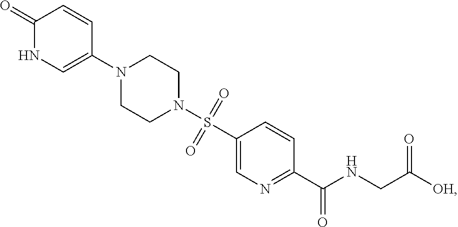 [SMILES: O=C(O)CNC(=O)c1ccc(S(=O)(=O)N2CCN(c3ccc(=O)[nH]c3)CC2)cn1] | (bl) |
| 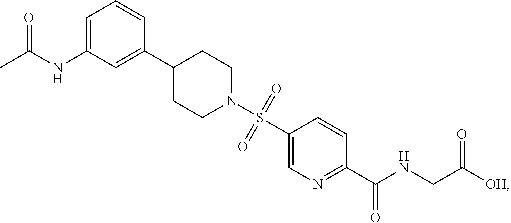 [SMILES: CC(=O)Nc1cccc(C2CCN(S(=O)(=O)c3ccc(C(=O)NCC(=O)O)nc3)CC2)c1] | (bm) |
| 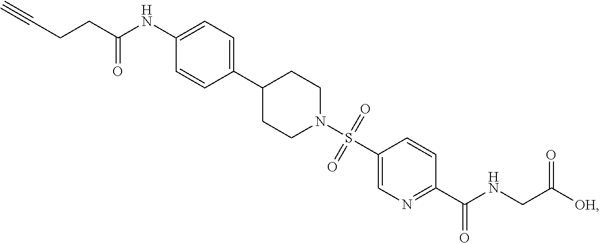 [SMILES: C#CCCC(=O)Nc1ccc(C2CCN(S(=O)(=O)c3ccc(C(=O)NCC(=O)O)nc3)CC2)cc1] | (bn) |
| 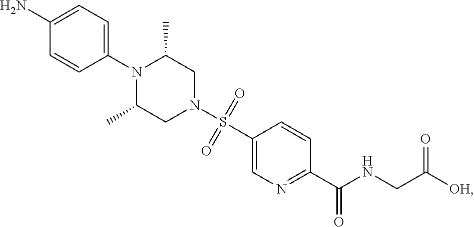 [SMILES: C[C@@H]1CN(S(=O)(=O)c2ccc(C(=O)NCC(=O)O)nc2)C[C@H](C)N1c1ccc(N)cc1] | (bo) |
| 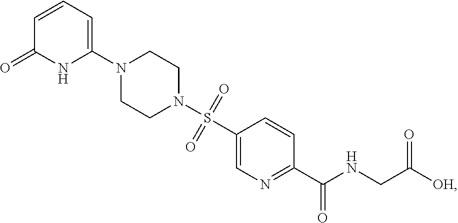 [SMILES: O=C(O)CNC(=O)c1ccc(S(=O)(=O)N2CCN(c3cccc(=O)[nH]3)CC2)cn1] | (bp) |
| 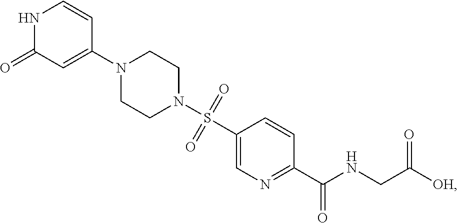 [SMILES: O=C(O)CNC(=O)c1ccc(S(=O)(=O)N2CCN(c3cc[nH]c(=O)c3)CC2)cn1] | (bq) |
| 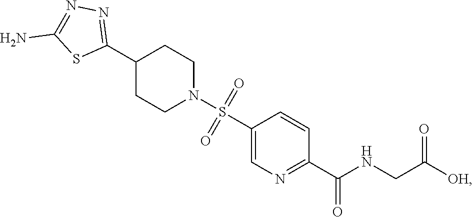 [SMILES: Nc1nnc(C2CCN(S(=O)(=O)c3ccc(C(=O)NCC(=O)O)nc3)CC2)s1] | (br) |
| 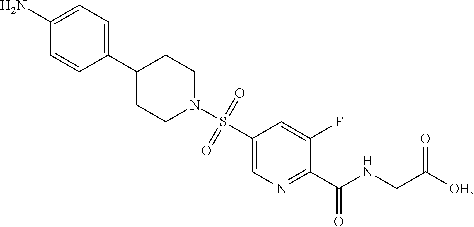 [SMILES: Nc1ccc(C2CCN(S(=O)(=O)c3cnc(C(=O)NCC(=O)O)c(F)c3)CC2)cc1] | (bs) |
| 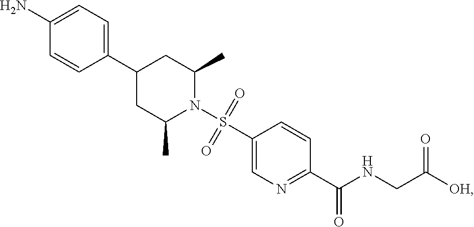 [SMILES: C[C@@H]1CC(c2ccc(N)cc2)C[C@H](C)N1S(=O)(=O)c1ccc(C(=O)NCC(=O)O)nc1] | (bt) |
| 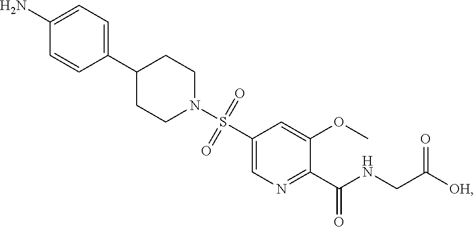 [SMILES: COc1cc(S(=O)(=O)N2CCC(c3ccc(N)cc3)CC2)cnc1C(=O)NCC(=O)O] | (bu) |
| 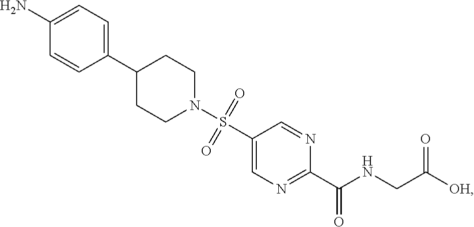 [SMILES: Nc1ccc(C2CCN(S(=O)(=O)c3cnc(C(=O)NCC(=O)O)nc3)CC2)cc1] | (bv) |
| 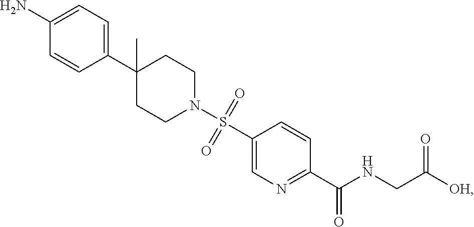 [SMILES: CC1(c2ccc(N)cc2)CCN(S(=O)(=O)c2ccc(C(=O)NCC(=O)O)nc2)CC1] | (bw) |
| 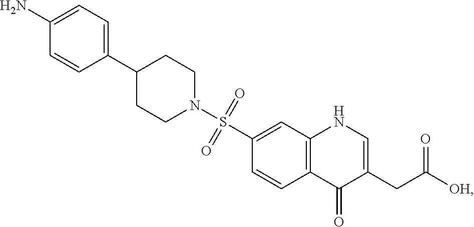 [SMILES: Nc1ccc(C2CCN(S(=O)(=O)c3ccc4c(=O)c(CC(=O)O)c[nH]c4c3)CC2)cc1] | (bx) |
| 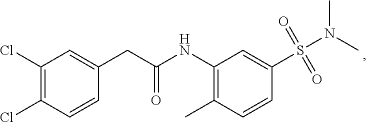 [SMILES: Cc1ccc(S(=O)(=O)N(C)C)cc1NC(=O)Cc1ccc(Cl)c(Cl)c1] | (by) |
| 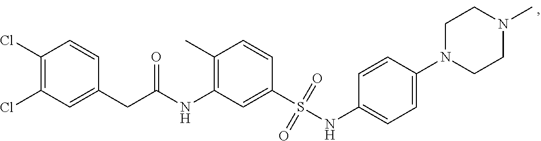 [SMILES: Cc1ccc(S(=O)(=O)Nc2ccc(N3CCN(C)CC3)cc2)cc1NC(=O)Cc1ccc(Cl)c(Cl)c1] | (bz) |
| 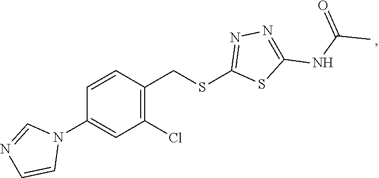 [SMILES: CC(=O)Nc1nnc(SCc2ccc(-n3ccnc3)cc2Cl)s1] | (ca) |
| 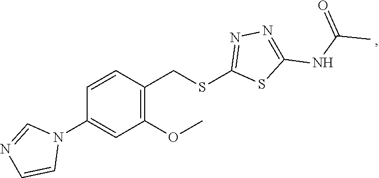 [SMILES: COc1cc(-n2ccnc2)ccc1CSc1nnc(NC(C)=O)s1] | (cb) |
| 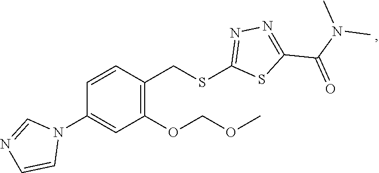 [SMILES: COCOc1cc(-n2ccnc2)ccc1CSc1nnc(C(=O)N(C)C)s1] | (cc) |
| 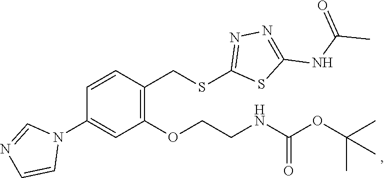 [SMILES: CC(=O)Nc1nnc(SCc2ccc(-n3ccnc3)cc2OCCNC(=O)OC(C)(C)C)s1] | (cd) |
| 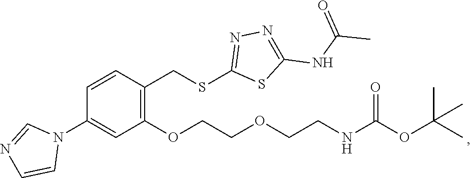 [SMILES: CC(=O)Nc1nnc(SCc2ccc(-n3ccnc3)cc2OCCOCCNC(=O)OC(C)(C)C)s1] | (ce) |
| 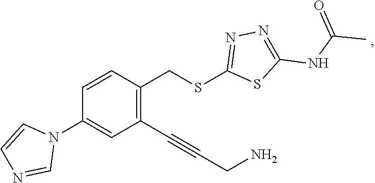 [SMILES: CC(=O)Nc1nnc(SCc2ccc(-n3ccnc3)cc2C#CCN)s1] | (cf) |
| 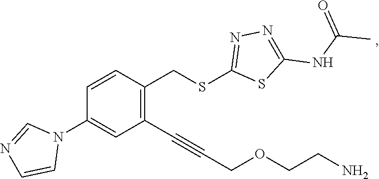 [SMILES: CC(=O)Nc1nnc(SCc2ccc(-n3ccnc3)cc2C#CCOCCN)s1] | (cg) |
| 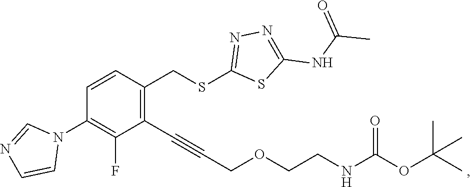 [SMILES: CC(=O)Nc1nnc(SCc2ccc(-n3ccnc3)c(F)c2C#CCOCCNC(=O)OC(C)(C)C)s1] | (ch) |
| 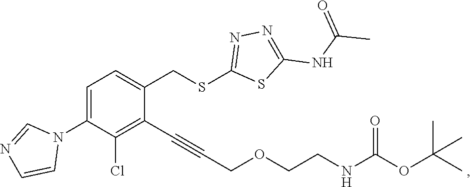 [SMILES: CC(=O)Nc1nnc(SCc2ccc(-n3ccnc3)c(Cl)c2C#CCOCCNC(=O)OC(C)(C)C)s1] | (ci) |
| 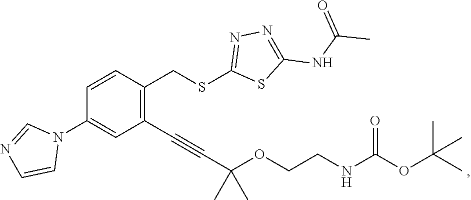 [SMILES: CC(=O)Nc1nnc(SCc2ccc(-n3ccnc3)cc2C#CC(C)(C)OCCNC(=O)OC(C)(C)C)s1] | (cj) |
| 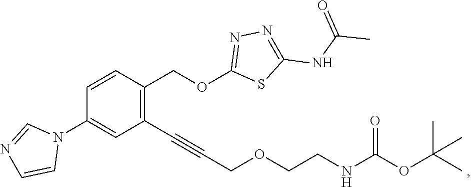 [SMILES: CC(=O)Nc1nnc(OCc2ccc(-n3ccnc3)cc2C#CCOCCNC(=O)OC(C)(C)C)s1] | (ck) |
| 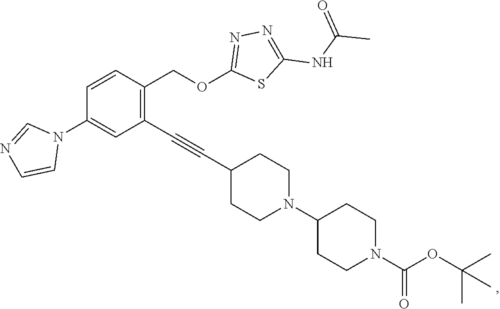 [SMILES: CC(=O)Nc1nnc(OCc2ccc(-n3ccnc3)cc2C#CC2CCN(C3CCN(C(=O)OC(C)(C)C)CC3)CC2)s1] | (cl) |
| 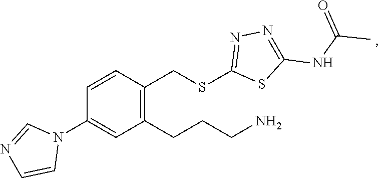 [SMILES: CC(=O)Nc1nnc(SCc2ccc(-n3ccnc3)cc2CCCN)s1] | (cm) |
| 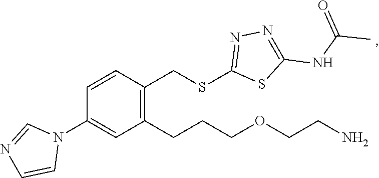 [SMILES: CC(=O)Nc1nnc(SCc2ccc(-n3ccnc3)cc2CCCOCCN)s1] | (cn) |
| 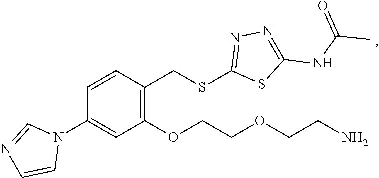 [SMILES: CC(=O)Nc1nnc(SCc2ccc(-n3ccnc3)cc2OCCOCCN)s1] | (co) |
| 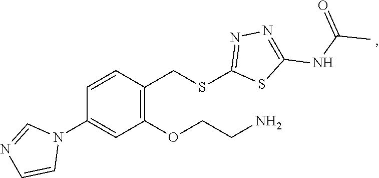 [SMILES: CC(=O)Nc1nnc(SCc2ccc(-n3ccnc3)cc2OCCN)s1] | (cp) |
| 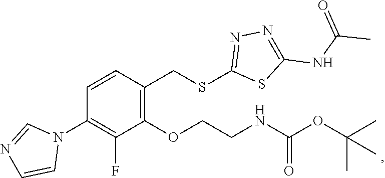 [SMILES: CC(=O)Nc1nnc(SCc2ccc(-n3ccnc3)c(F)c2OCCNC(=O)OC(C)(C)C)s1] | (cq) |
| 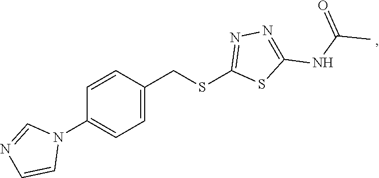 [SMILES: CC(=O)Nc1nnc(SCc2ccc(-n3ccnc3)cc2)s1] | (cr) |
| 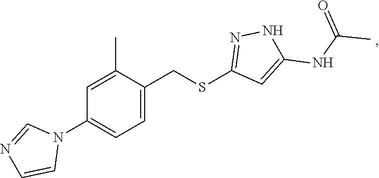 [SMILES: CC(=O)Nc1cc(SCc2ccc(-n3ccnc3)cc2C)n[nH]1] | (cs) |
| 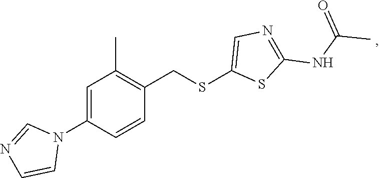 [SMILES: CC(=O)Nc1ncc(SCc2ccc(-n3ccnc3)cc2C)s1] | (ct) |
| 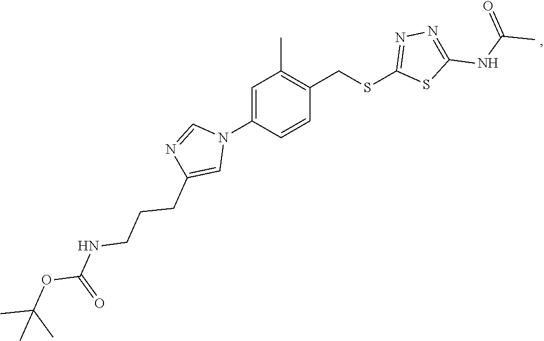 [SMILES: CC(=O)Nc1nnc(SCc2ccc(-n3cnc(CCCNC(=O)OC(C)(C)C)c3)cc2C)s1] | (cu) |
| 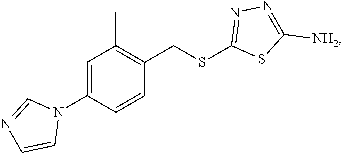 [SMILES: Cc1cc(-n2ccnc2)ccc1CSc1nnc(N)s1] | (cv) |
| 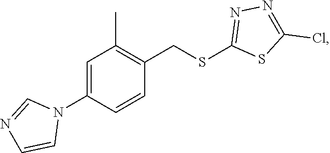 [SMILES: Cc1cc(-n2ccnc2)ccc1CSc1nnc(Cl)s1] | (cw) |
| 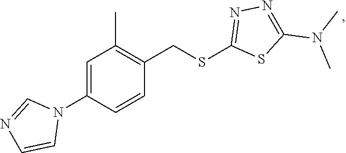 [SMILES: Cc1cc(-n2ccnc2)ccc1CSc1nnc(N(C)C)s1] | (cx) |
| 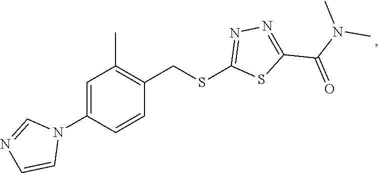 [SMILES: Cc1cc(-n2ccnc2)ccc1CSc1nnc(C(=O)N(C)C)s1] | (cy) |
| 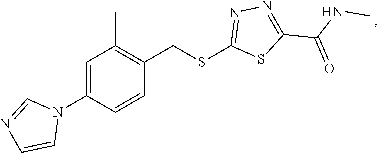 [SMILES: CNC(=O)c1nnc(SCc2ccc(-n3ccnc3)cc2C)s1] | (cz) |
| 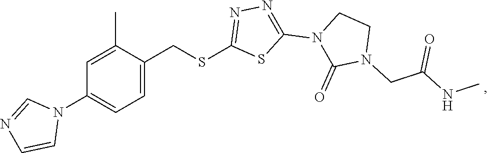 [SMILES: CNC(=O)CN1CCN(c2nnc(SCc3ccc(-n4ccnc4)cc3C)s2)C1=O] | (da) |
| 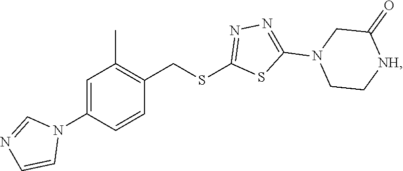 [SMILES: Cc1cc(-n2ccnc2)ccc1CSc1nnc(N2CCNC(=O)C2)s1] | (db) |
| 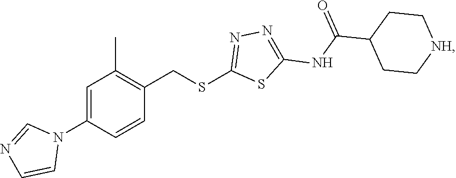 [SMILES: Cc1cc(-n2ccnc2)ccc1CSc1nnc(NC(=O)C2CCNCC2)s1] | (dc) |
| 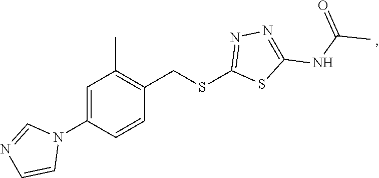 [SMILES: CC(=O)Nc1nnc(SCc2ccc(-n3ccnc3)cc2C)s1] | (dd) |
| 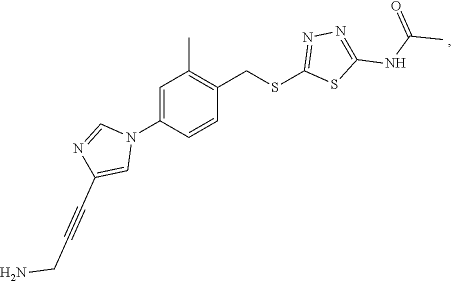 [SMILES: CC(=O)Nc1nnc(SCc2ccc(-n3cnc(C#CCN)c3)cc2C)s1] | (de) |
| 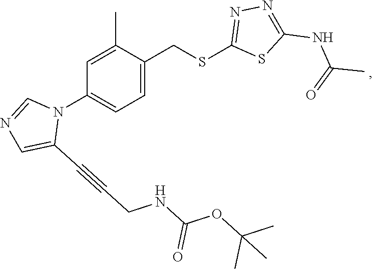 [SMILES: CC(=O)Nc1nnc(SCc2ccc(-n3cncc3C#CCNC(=O)OC(C)(C)C)cc2C)s1] | (df) |
| 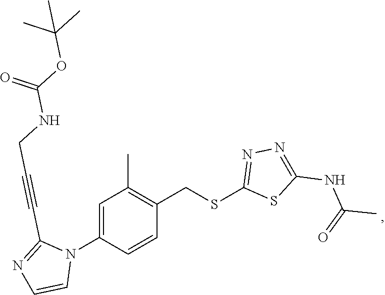 [SMILES: CC(=O)Nc1nnc(SCc2ccc(-n3ccnc3C#CCNC(=O)OC(C)(C)C)cc2C)s1] | (dg) |
| 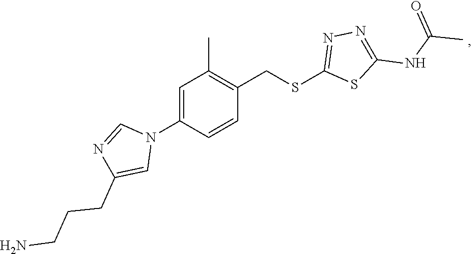 [SMILES: CC(=O)Nc1nnc(SCc2ccc(-n3cnc(CCCN)c3)cc2C)s1] | (dh) |
| 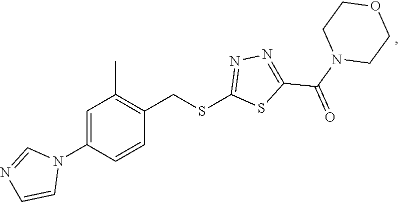 [SMILES: Cc1cc(-n2ccnc2)ccc1CSc1nnc(C(=O)N2CCOCC2)s1] | (di) |
| 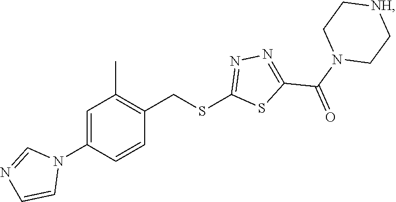 [SMILES: Cc1cc(-n2ccnc2)ccc1CSc1nnc(C(=O)N2CCNCC2)s1] | (dj) |
| 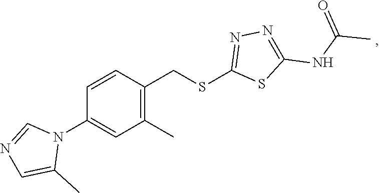 [SMILES: CC(=O)Nc1nnc(SCc2ccc(-n3cncc3C)cc2C)s1] | (dk) |
| 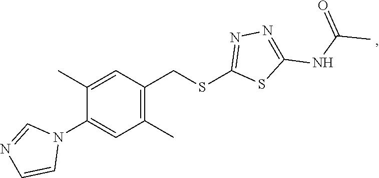 [SMILES: CC(=O)Nc1nnc(SCc2cc(C)c(-n3ccnc3)cc2C)s1] | (dl) |
| 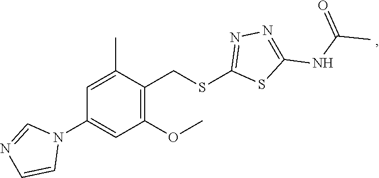 [SMILES: COc1cc(-n2ccnc2)cc(C)c1CSc1nnc(NC(C)=O)s1] | (dm) |
| 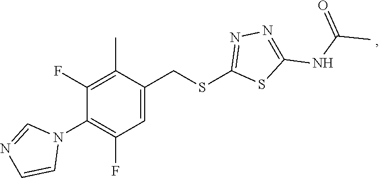 [SMILES: CC(=O)Nc1nnc(SCc2cc(F)c(-n3ccnc3)c(F)c2C)s1] | (dn) |
| 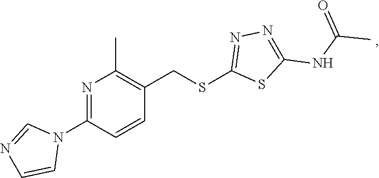 [SMILES: CC(=O)Nc1nnc(SCc2ccc(-n3ccnc3)nc2C)s1] | (do) |
| 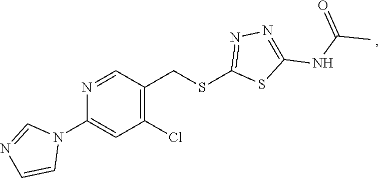 [SMILES: CC(=O)Nc1nnc(SCc2cnc(-n3ccnc3)cc2Cl)s1] | (dp) |
| 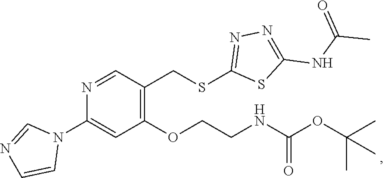 [SMILES: CC(=O)Nc1nnc(SCc2cnc(-n3ccnc3)cc2OCCNC(=O)OC(C)(C)C)s1] | (dq) |
| 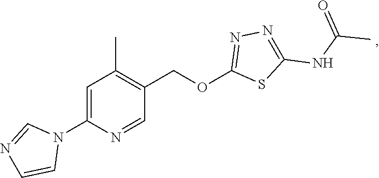 [SMILES: CC(=O)Nc1nnc(OCc2cnc(-n3ccnc3)cc2C)s1] | (dr) |
| 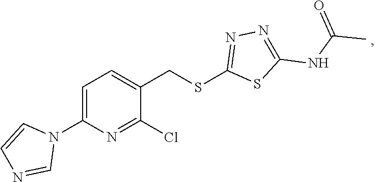 [SMILES: CC(=O)Nc1nnc(SCc2ccc(-n3ccnc3)nc2Cl)s1] | (ds) |
| 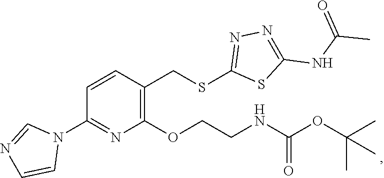 [SMILES: CC(=O)Nc1nnc(SCc2ccc(-n3ccnc3)nc2OCCNC(=O)OC(C)(C)C)s1] | (dt) |
| 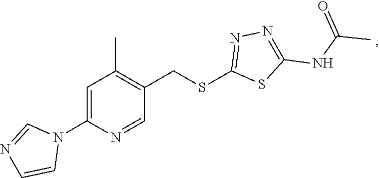 [SMILES: CC(=O)Nc1nnc(SCc2cnc(-n3ccnc3)cc2C)s1] | (du) |
| 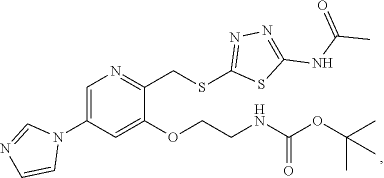 [SMILES: CC(=O)Nc1nnc(SCc2ncc(-n3ccnc3)cc2OCCNC(=O)OC(C)(C)C)s1] | (dv) |
| 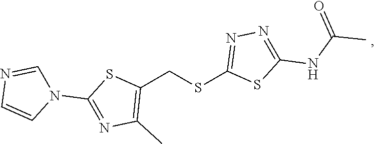 [SMILES: CC(=O)Nc1nnc(SCc2sc(-n3ccnc3)nc2C)s1] | (dw) |
| 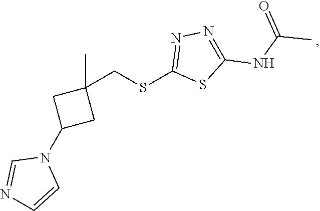 [SMILES: CC(=O)Nc1nnc(SCC2(C)CC(n3ccnc3)C2)s1] | (dx) |
| 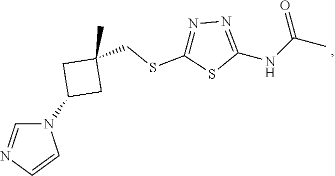 [SMILES: CC(=O)Nc1nnc(SC[C@]2(C)C[C@H](n3ccnc3)C2)s1] | (dy) |
| 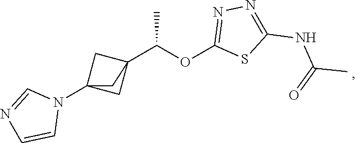 [SMILES: CC(=O)Nc1nnc(O[C@@H](C)C23CC(n4ccnc4)(C2)C3)s1] | (dz) |
| 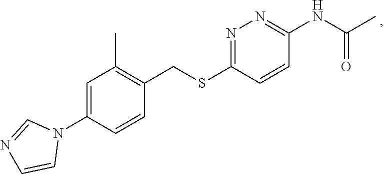 [SMILES: CC(=O)Nc1ccc(SCc2ccc(-n3ccnc3)cc2C)nn1] | (ea) |
| 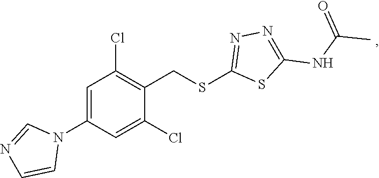 [SMILES: CC(=O)Nc1nnc(SCc2c(Cl)cc(-n3ccnc3)cc2Cl)s1] | (eb) |
| 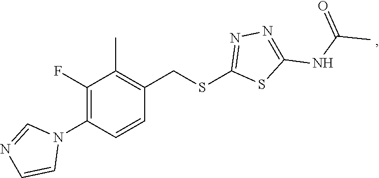 [SMILES: CC(=O)Nc1nnc(SCc2ccc(-n3ccnc3)c(F)c2C)s1] | (ec) |
| 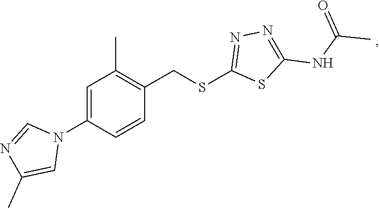 [SMILES: CC(=O)Nc1nnc(SCc2ccc(-n3cnc(C)c3)cc2C)s1] | (ed) |
| 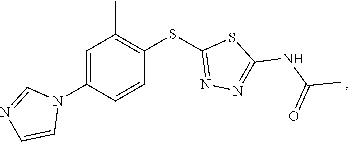 [SMILES: CC(=O)Nc1nnc(Sc2ccc(-n3ccnc3)cc2C)s1] | (ee) |
| 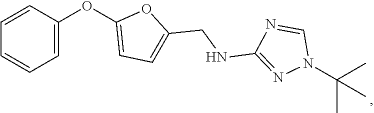 [SMILES: CC(C)(C)n1cnc(NCc2ccc(Oc3ccccc3)o2)n1] | (ef) |
| 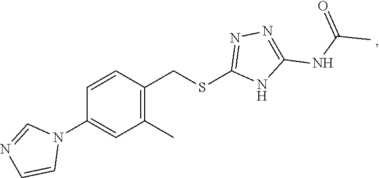 [SMILES: CC(=O)Nc1nnc(SCc2ccc(-n3ccnc3)cc2C)[nH]1] | (eg) |
| 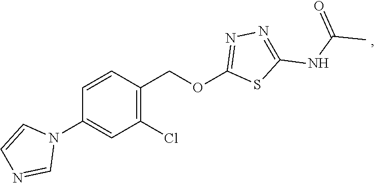 [SMILES: CC(=O)Nc1nnc(OCc2ccc(-n3ccnc3)cc2Cl)s1] | (eh) |
| 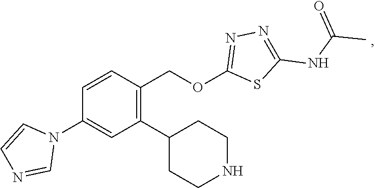 [SMILES: CC(=O)Nc1nnc(OCc2ccc(-n3ccnc3)cc2C2CCNCC2)s1] | (ei) |
| 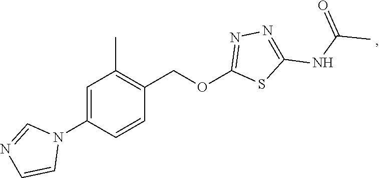 [SMILES: CC(=O)Nc1nnc(OCc2ccc(-n3ccnc3)cc2C)s1] | (ej) |
| 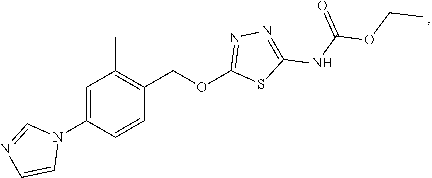 [SMILES: CCOC(=O)Nc1nnc(OCc2ccc(-n3ccnc3)cc2C)s1] | (ek) |
| 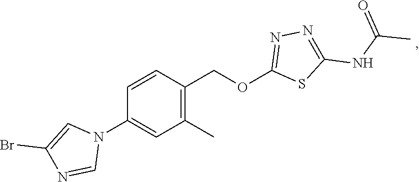 [SMILES: CC(=O)Nc1nnc(OCc2ccc(-n3cnc(Br)c3)cc2C)s1] | (el) |
| 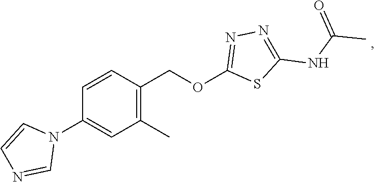 [SMILES: CC(=O)Nc1nnc(OCc2ccc(-n3ccnc3)cc2C)s1] | (em) |
| 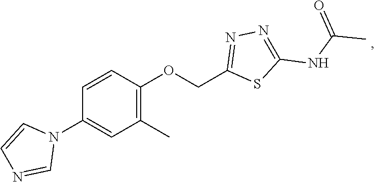 [SMILES: CC(=O)Nc1nnc(COc2ccc(-n3ccnc3)cc2C)s1] | (en) |
| 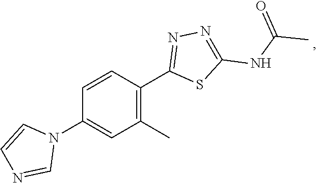 [SMILES: CC(=O)Nc1nnc(-c2ccc(-n3ccnc3)cc2C)s1] | (eo) |
| 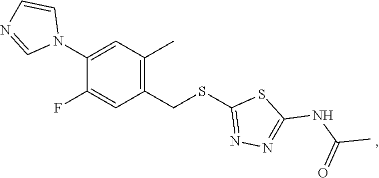 [SMILES: CC(=O)Nc1nnc(SCc2cc(F)c(-n3ccnc3)cc2C)s1] | (ep) |
| 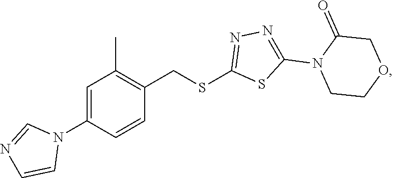 [SMILES: Cc1cc(-n2ccnc2)ccc1CSc1nnc(N2CCOCC2=O)s1] | (eq) |
| 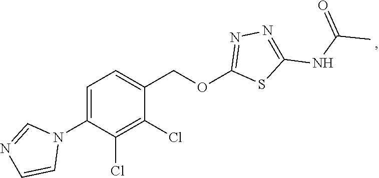 [SMILES: CC(=O)Nc1nnc(OCc2ccc(-n3ccnc3)c(Cl)c2Cl)s1] | (er) |
| 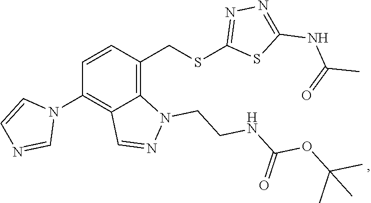 [SMILES: CC(=O)Nc1nnc(SCc2ccc(-n3ccnc3)c3cnn(CCNC(=O)OC(C)(C)C)c23)s1] | (es) |
| 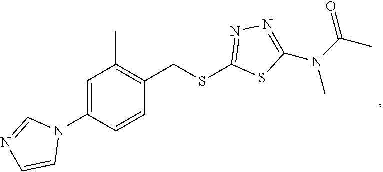 [SMILES: CC(=O)N(C)c1nnc(SCc2ccc(-n3ccnc3)cc2C)s1] | (et) |
| 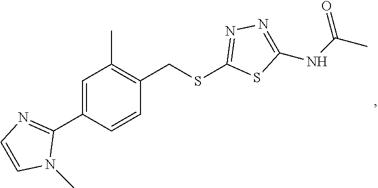 [SMILES: CC(=O)Nc1nnc(SCc2ccc(-c3nccn3C)cc2C)s1] | (eu) |
| 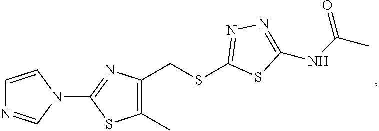 [SMILES: CC(=O)Nc1nnc(SCc2nc(-n3ccnc3)sc2C)s1] | (ev) |
| 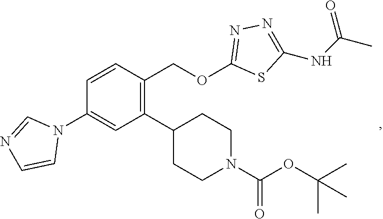 [SMILES: CC(=O)Nc1nnc(OCc2ccc(-n3ccnc3)cc2C2CCN(C(=O)OC(C)(C)C)CC2)s1] | (ew) |
| 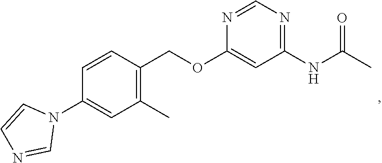 [SMILES: CC(=O)Nc1cc(OCc2ccc(-n3ccnc3)cc2C)ncn1] | (ex) |
| 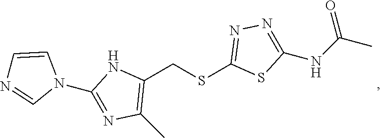 [SMILES: CC(=O)Nc1nnc(SCc2[nH]c(-n3ccnc3)nc2C)s1] | (ey) |
| 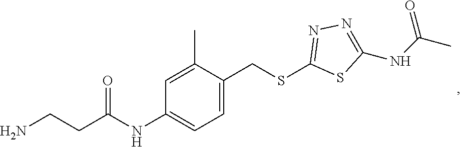 [SMILES: CC(=O)Nc1nnc(SCc2ccc(NC(=O)CCN)cc2C)s1] | (ez) |
| 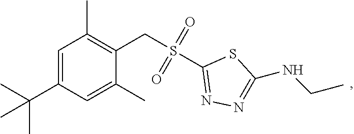 [SMILES: CCNc1nnc(S(=O)(=O)Cc2c(C)cc(C(C)(C)C)cc2C)s1] | (fa) |
| 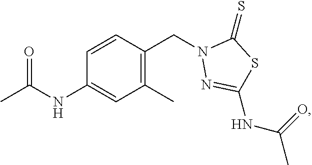 [SMILES: CC(=O)Nc1ccc(Cn2nc(NC(C)=O)sc2=S)c(C)c1] | (fb) |
| 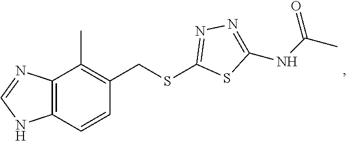 [SMILES: CC(=O)Nc1nnc(SCc2ccc3[nH]cnc3c2C)s1] | (fc) |
| 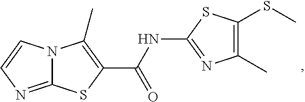 [SMILES: CSc1sc(NC(=O)c2sc3nccn3c2C)nc1C] | (fd) |
| 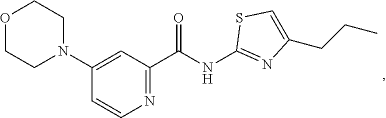 [SMILES: CCCc1csc(NC(=O)c2cc(N3CCOCC3)ccn2)n1] | (fe) |
| 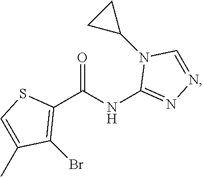 [SMILES: Cc1csc(C(=O)Nc2nncn2C2CC2)c1Br] | (ff) |
| 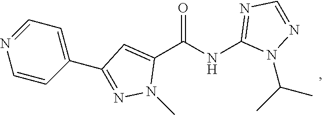 [SMILES: CC(C)n1ncnc1NC(=O)c1cc(-c2ccncc2)nn1C] | (fg) |
| 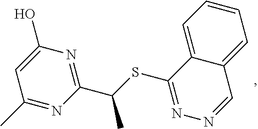 [SMILES: Cc1cc(O)nc([C@H](C)Sc2nncc3ccccc23)n1] | (fh) |
| 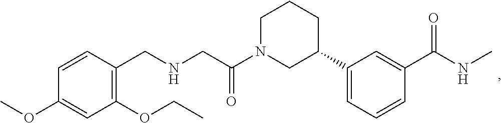 | (fi) |
| 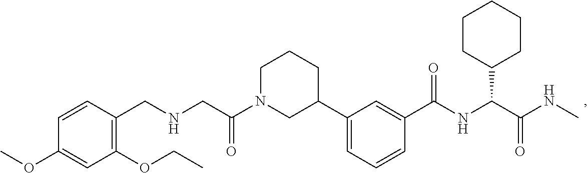 | (fj) |
| 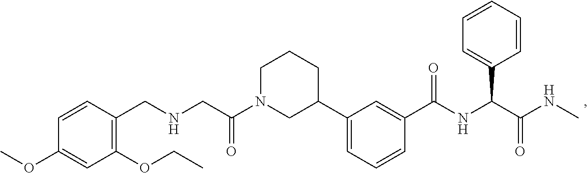 | (fk) |
| 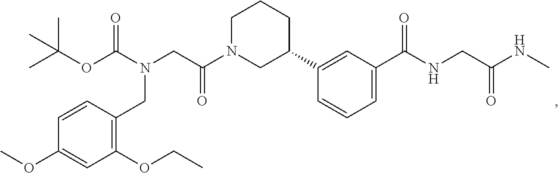 | (fl) |
| 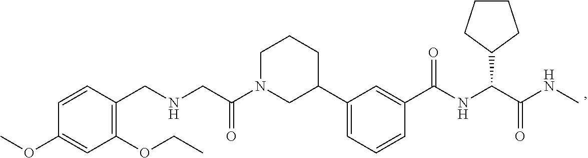 | (fm) |
| 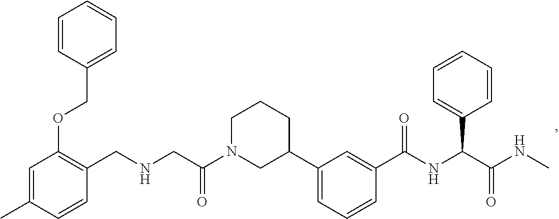 | (fn) |
| 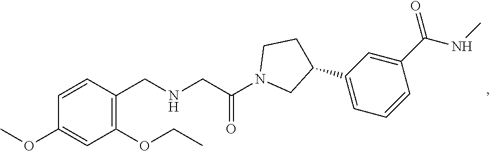 | (fo) |
| 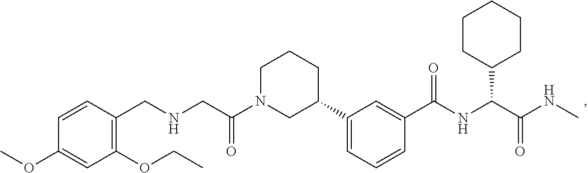 | (fp) |
| 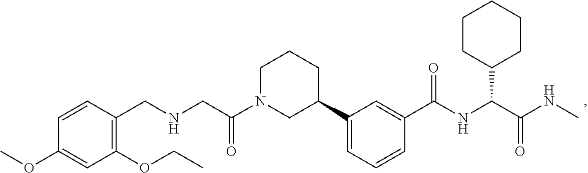 | (fq) |
| 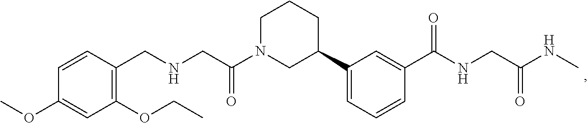 | (fr) |
| 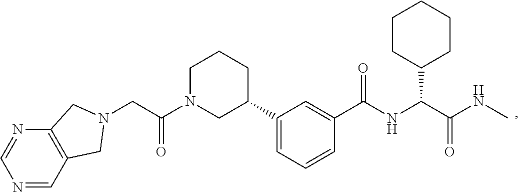 [SMILES: CNC(=O)[C@H](NC(=O)c1cccc([C@H]2CCCN(C(=O)CN3Cc4cncnc4C3)C2)c1)C1CCCCC1] | (fs) |
| 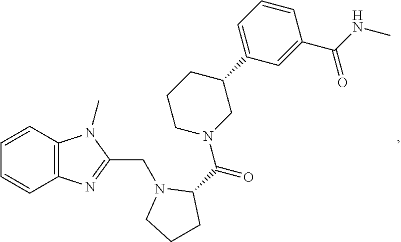 [SMILES: CNC(=O)c1cccc([C@H]2CCCN(C(=O)[C@@H]3CCCN3Cc3nc4ccccc4n3C)C2)c1] | (ft) |
| 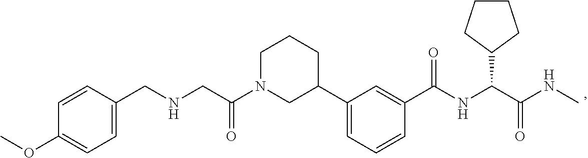 | (fu) |
| 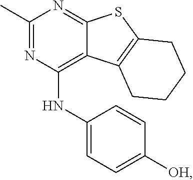 [SMILES: Cc1nc(Nc2ccc(O)cc2)c2c3c(sc2n1)CCCC3] | (fv) |
| 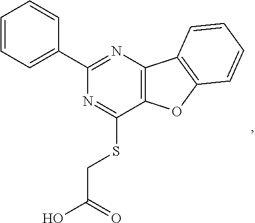 [SMILES: O=C(O)CSc1nc(-c2ccccc2)nc2c1oc1ccccc12] | (fw) |
| 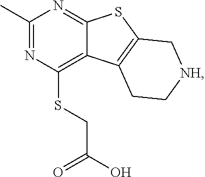 [SMILES: Cc1nc(SCC(=O)O)c2c3c(sc2n1)CNCC3] | (fx) |
| 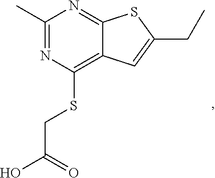 [SMILES: CCc1cc2c(SCC(=O)O)nc(C)nc2s1] | (fy) |
| 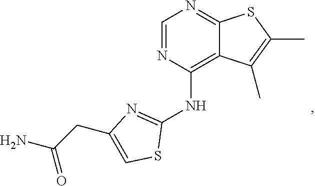 [SMILES: Cc1sc2ncnc(Nc3nc(CC(N)=O)cs3)c2c1C] | (fz) |
| 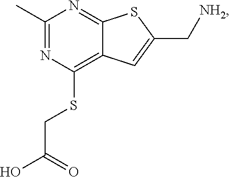 [SMILES: Cc1nc(SCC(=O)O)c2cc(CN)sc2n1] | (ga) |
| 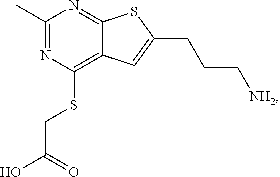 [SMILES: Cc1nc(SCC(=O)O)c2cc(CCCN)sc2n1] | (gb) |
| 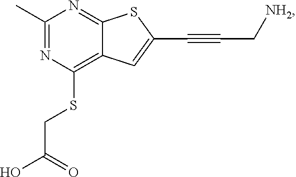 [SMILES: Cc1nc(SCC(=O)O)c2cc(C#CCN)sc2n1] | (gc) |
| 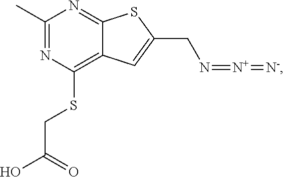 | (gd) |
| 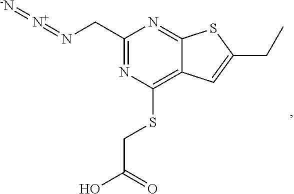 | (ge) |
| 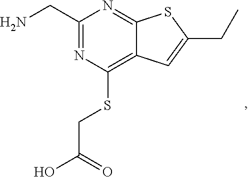 [SMILES: CCc1cc2c(SCC(=O)O)nc(CN)nc2s1] | (gf) |
| 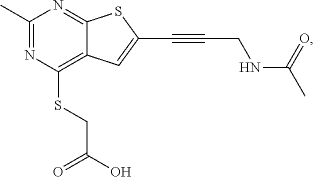 [SMILES: CC(=O)NCC#Cc1cc2c(SCC(=O)O)nc(C)nc2s1] | (gg) |
| 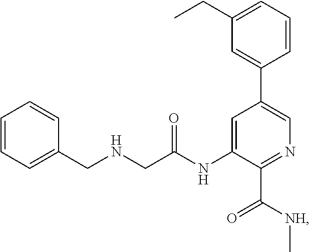 [SMILES: CCc1cccc(-c2cnc(C(=O)NC)c(NC(=O)CNCc3ccccc3)c2)c1] | (hf) |
| 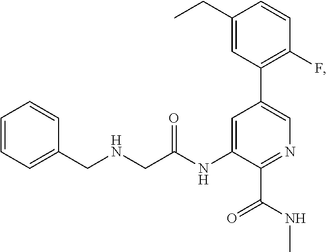 [SMILES: CCc1ccc(F)c(-c2cnc(C(=O)NC)c(NC(=O)CNCc3ccccc3)c2)c1] | (hg) |
| 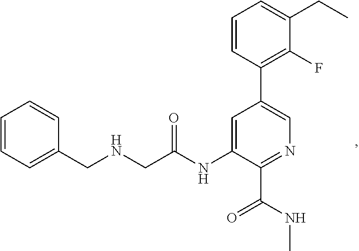 [SMILES: CCc1cccc(-c2cnc(C(=O)NC)c(NC(=O)CNCc3ccccc3)c2)c1F] | (hh) |
| 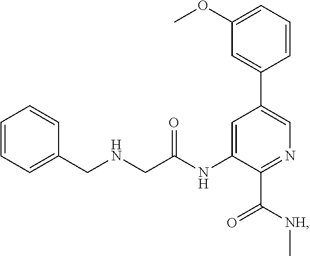 [SMILES: CNC(=O)c1ncc(-c2cccc(OC)c2)cc1NC(=O)CNCc1ccccc1] | (hi) |
| 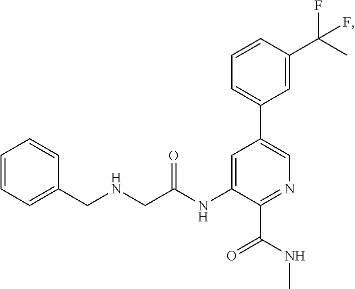 [SMILES: CNC(=O)c1ncc(-c2cccc(C(C)(F)F)c2)cc1NC(=O)CNCc1ccccc1] | (hj) |
| 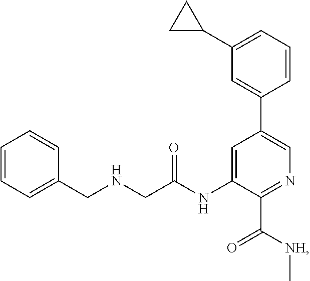 [SMILES: CNC(=O)c1ncc(-c2cccc(C3CC3)c2)cc1NC(=O)CNCc1ccccc1] | (hk) |
| 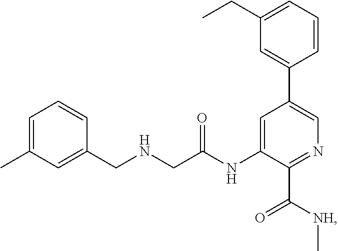 [SMILES: CCc1cccc(-c2cnc(C(=O)NC)c(NC(=O)CNCc3cccc(C)c3)c2)c1] | (hl) |
| 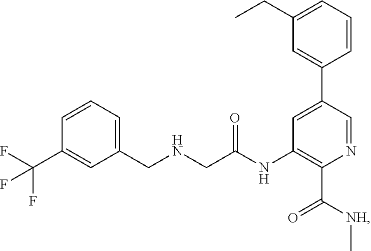 [SMILES: CCc1cccc(-c2cnc(C(=O)NC)c(NC(=O)CNCc3cccc(C(F)(F)F)c3)c2)c1] | (hm) |
| 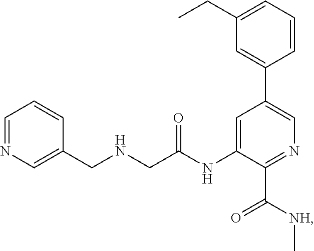 [SMILES: CCc1cccc(-c2cnc(C(=O)NC)c(NC(=O)CNCc3cccnc3)c2)c1] | (hn) |
| 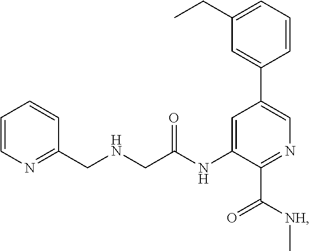 [SMILES: CCc1cccc(-c2cnc(C(=O)NC)c(NC(=O)CNCc3ccccn3)c2)c1] | (ho) |
| 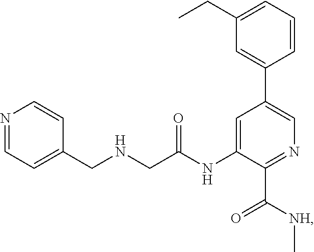 [SMILES: CCc1cccc(-c2cnc(C(=O)NC)c(NC(=O)CNCc3ccncc3)c2)c1] | (hp) |
| 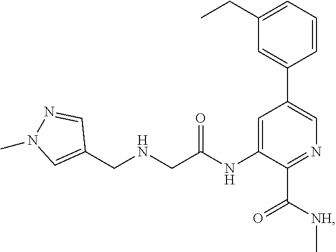 [SMILES: CCc1cccc(-c2cnc(C(=O)NC)c(NC(=O)CNCc3cnn(C)c3)c2)c1] | (hq) |
| 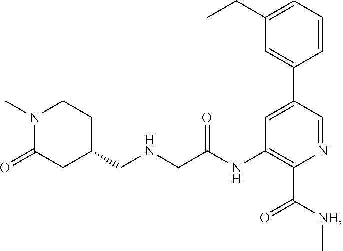 [SMILES: CCc1cccc(-c2cnc(C(=O)NC)c(NC(=O)CNC[C@H]3CCN(C)C(=O)C3)c2)c1] | (hr) |
| 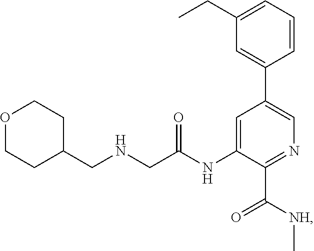 [SMILES: CCc1cccc(-c2cnc(C(=O)NC)c(NC(=O)CNCC3CCOCC3)c2)c1] | (hs) |
| 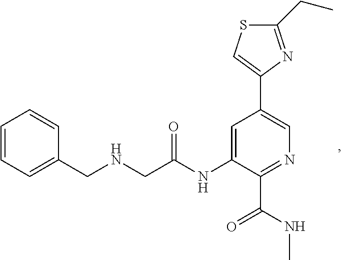 [SMILES: CCc1nc(-c2cnc(C(=O)NC)c(NC(=O)CNCc3ccccc3)c2)cs1] | (ht) |
| 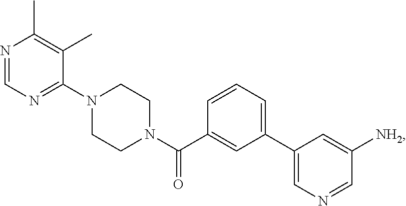 [SMILES: Cc1ncnc(N2CCN(C(=O)c3cccc(-c4cncc(N)c4)c3)CC2)c1C] | (hu) |
| 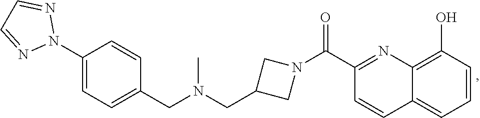 [SMILES: CN(Cc1ccc(-n2nccn2)cc1)CC1CN(C(=O)c2ccc3cccc(O)c3n2)C1] | (hv) |
| 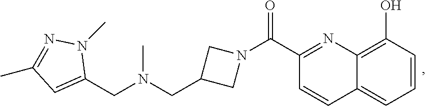 [SMILES: Cc1cc(CN(C)CC2CN(C(=O)c3ccc4cccc(O)c4n3)C2)n(C)n1] | (hw) |
| 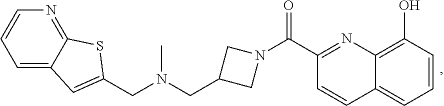 [SMILES: CN(Cc1cc2cccnc2s1)CC1CN(C(=O)c2ccc3cccc(O)c3n2)C1] | (hx) |
| 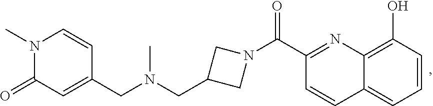 [SMILES: CN(Cc1ccn(C)c(=O)c1)CC1CN(C(=O)c2ccc3cccc(O)c3n2)C1] | (hy) |
| 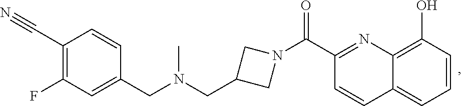 [SMILES: CN(Cc1ccc(C#N)c(F)c1)CC1CN(C(=O)c2ccc3cccc(O)c3n2)C1] | (hz) |
| 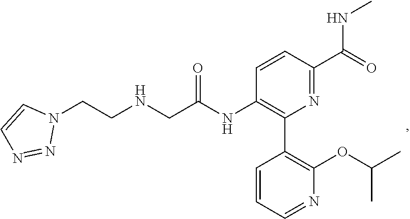 [SMILES: CNC(=O)c1ccc(NC(=O)CNCCn2ccnn2)c(-c2cccnc2OC(C)C)n1] | (ia) |
| 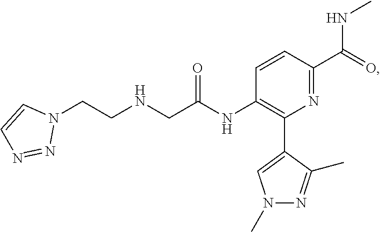 [SMILES: CNC(=O)c1ccc(NC(=O)CNCCn2ccnn2)c(-c2cn(C)nc2C)n1] | (ib) |
| 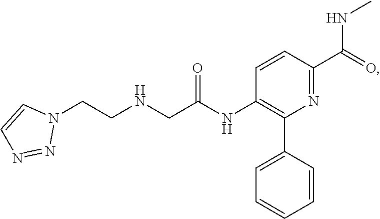 [SMILES: CNC(=O)c1ccc(NC(=O)CNCCn2ccnn2)c(-c2ccccc2)n1] | (ic) |
| 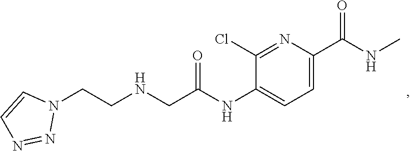 [SMILES: CNC(=O)c1ccc(NC(=O)CNCCn2ccnn2)c(Cl)n1] | (id) |
| 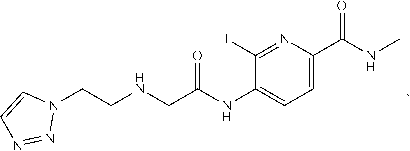 [SMILES: CNC(=O)c1ccc(NC(=O)CNCCn2ccnn2)c(I)n1] | (ie) |
| 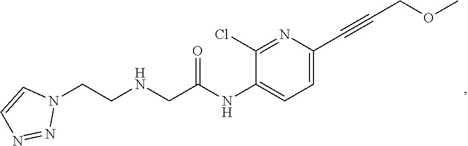 [SMILES: COCC#Cc1ccc(NC(=O)CNCCn2ccnn2)c(Cl)n1] | (if) |
| 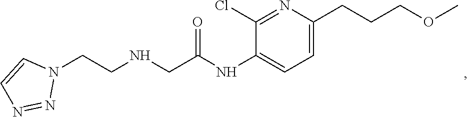 [SMILES: COCCCc1ccc(NC(=O)CNCCn2ccnn2)c(Cl)n1] | (ig) |
| 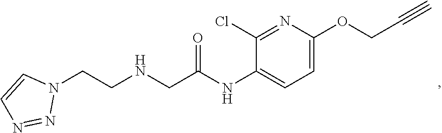 [SMILES: C#CCOc1ccc(NC(=O)CNCCn2ccnn2)c(Cl)n1] | (ih) |
| 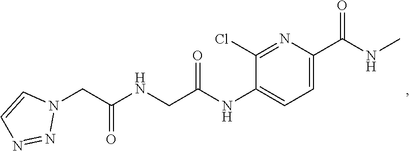 [SMILES: CNC(=O)c1ccc(NC(=O)CNC(=O)Cn2ccnn2)c(Cl)n1] | (ii) |
| 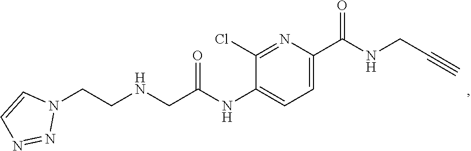 [SMILES: C#CCNC(=O)c1ccc(NC(=O)CNCCn2ccnn2)c(Cl)n1] | (ij) |
| 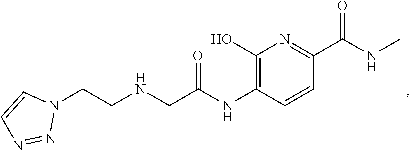 [SMILES: CNC(=O)c1ccc(NC(=O)CNCCn2ccnn2)c(O)n1] | (ik) |
| 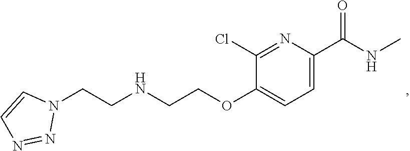 [SMILES: CNC(=O)c1ccc(OCCNCCn2ccnn2)c(Cl)n1] | (il) |
| 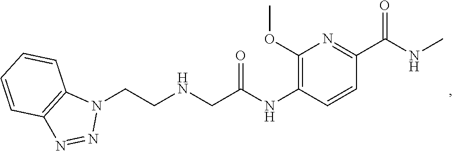 [SMILES: CNC(=O)c1ccc(NC(=O)CNCCn2nnc3ccccc32)c(OC)n1] | (im) |
| 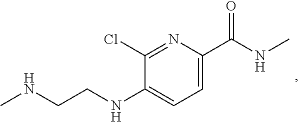 | (in) |
| 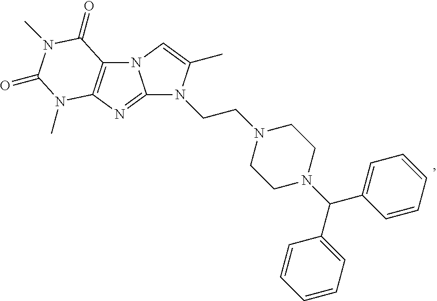 [SMILES: Cc1cn2c3c(=O)n(C)c(=O)n(C)c3nc2n1CCN1CCN(C(c2ccccc2)c2ccccc2)CC1] | (io) |
| 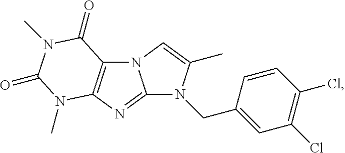 [SMILES: Cc1cn2c3c(=O)n(C)c(=O)n(C)c3nc2n1Cc1ccc(Cl)c(Cl)c1] | (ip) |
| 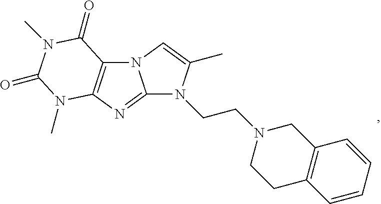 [SMILES: Cc1cn2c3c(=O)n(C)c(=O)n(C)c3nc2n1CCN1CCc2ccccc2C1] | (iq) |
| 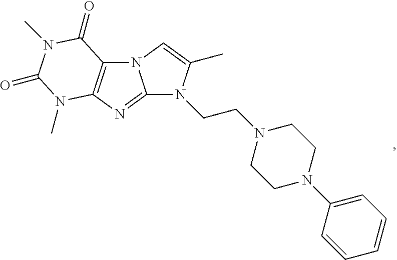 [SMILES: Cc1cn2c3c(=O)n(C)c(=O)n(C)c3nc2n1CCN1CCN(c2ccccc2)CC1] | (ir) |
| 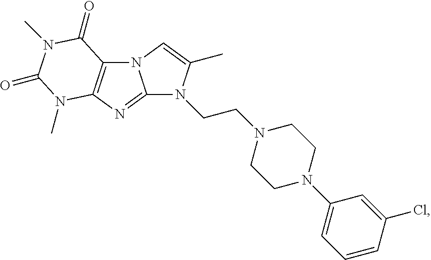 [SMILES: Cc1cn2c3c(=O)n(C)c(=O)n(C)c3nc2n1CCN1CCN(c2cccc(Cl)c2)CC1] | (is) |
| 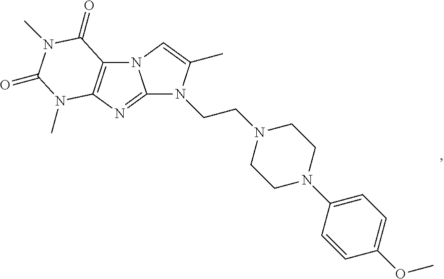 [SMILES: COc1ccc(N2CCN(CCn3c(C)cn4c5c(=O)n(C)c(=O)n(C)c5nc34)CC2)cc1] | (it) |
| 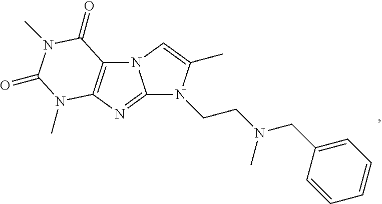 [SMILES: Cc1cn2c3c(=O)n(C)c(=O)n(C)c3nc2n1CCN(C)Cc1ccccc1] | (iu) |
| 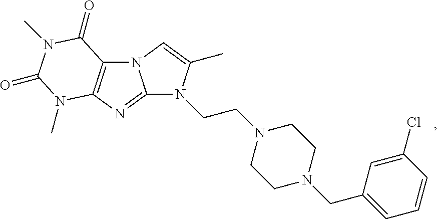 [SMILES: Cc1cn2c3c(=O)n(C)c(=O)n(C)c3nc2n1CCN1CCN(Cc2cccc(Cl)c2)CC1] | (iv) |
| 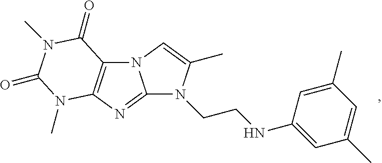 [SMILES: Cc1cc(C)cc(NCCn2c(C)cn3c4c(=O)n(C)c(=O)n(C)c4nc23)c1] | (iw) |
| 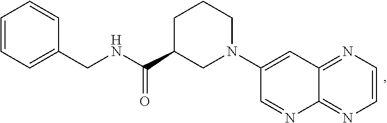 [SMILES: O=C(NCc1ccccc1)[C@H]1CCCN(c2cnc3nccnc3c2)C1] | (ix) |
| 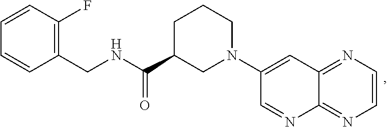 [SMILES: O=C(NCc1ccccc1F)[C@H]1CCCN(c2cnc3nccnc3c2)C1] | (iy) |
| 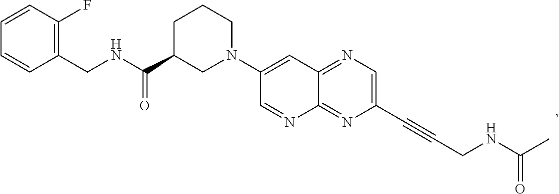 [SMILES: CC(=O)NCC#Cc1cnc2cc(N3CCC[C@H](C(=O)NCc4ccccc4F)C3)cnc2n1] | (iz) |
| 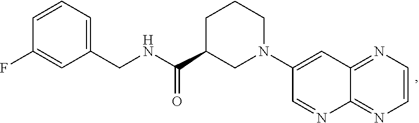 [SMILES: O=C(NCc1cccc(F)c1)[C@H]1CCCN(c2cnc3nccnc3c2)C1] | (ja) |
| 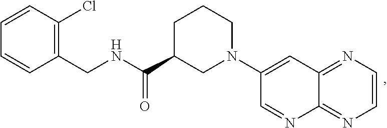 [SMILES: O=C(NCc1ccccc1Cl)[C@H]1CCCN(c2cnc3nccnc3c2)C1] | (jb) |
| 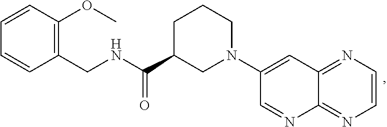 [SMILES: COc1ccccc1CNC(=O)[C@H]1CCCN(c2cnc3nccnc3c2)C1] | (jc) |
| 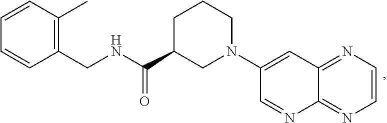 [SMILES: Cc1ccccc1CNC(=O)[C@H]1CCCN(c2cnc3nccnc3c2)C1] | (jd) |
| 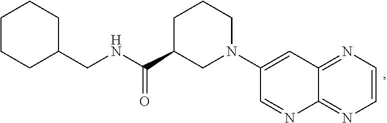 [SMILES: O=C(NCC1CCCCC1)[C@H]1CCCN(c2cnc3nccnc3c2)C1] | (je) |
| 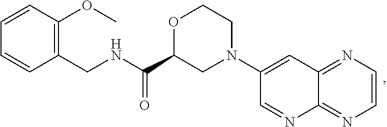 [SMILES: COc1ccccc1CNC(=O)[C@@H]1CN(c2cnc3nccnc3c2)CCO1] | (jf) |
| 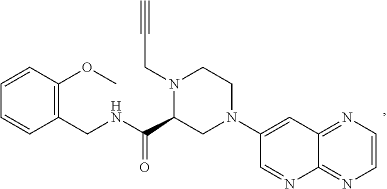 [SMILES: C#CCN1CCN(c2cnc3nccnc3c2)C[C@H]1C(=O)NCc1ccccc1OC] | (jg) |
| 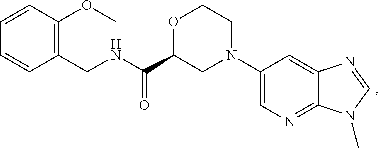 [SMILES: COc1ccccc1CNC(=O)[C@@H]1CN(c2cnc3c(c2)ncn3C)CCO1] | (jh) |
| 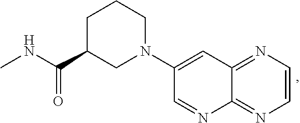 [SMILES: CNC(=O)[C@H]1CCCN(c2cnc3nccnc3c2)C1] | (ji) |
| 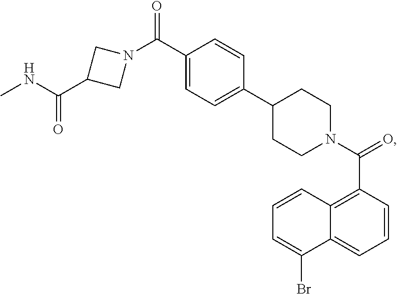 [SMILES: CNC(=O)C1CN(C(=O)c2ccc(C3CCN(C(=O)c4cccc5c(Br)cccc45)CC3)cc2)C1] | (jj) |
| 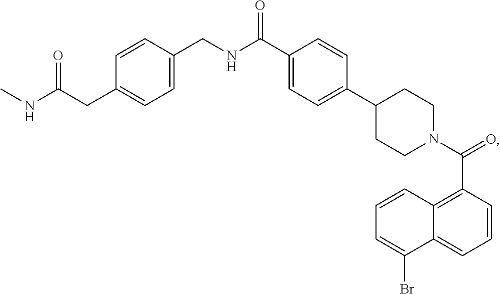 [SMILES: CNC(=O)Cc1ccc(CNC(=O)c2ccc(C3CCN(C(=O)c4cccc5c(Br)cccc45)CC3)cc2)cc1] | (jk) |
| 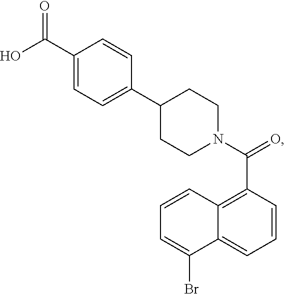 [SMILES: O=C(O)c1ccc(C2CCN(C(=O)c3cccc4c(Br)cccc34)CC2)cc1] | (jl) |
| 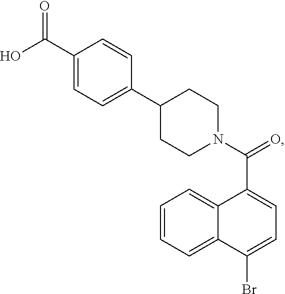 [SMILES: O=C(O)c1ccc(C2CCN(C(=O)c3ccc(Br)c4ccccc34)CC2)cc1] | (jm) |
| 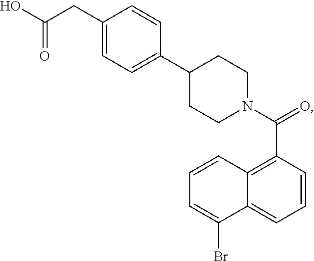 [SMILES: O=C(O)Cc1ccc(C2CCN(C(=O)c3cccc4c(Br)cccc34)CC2)cc1] | (jn) |
| 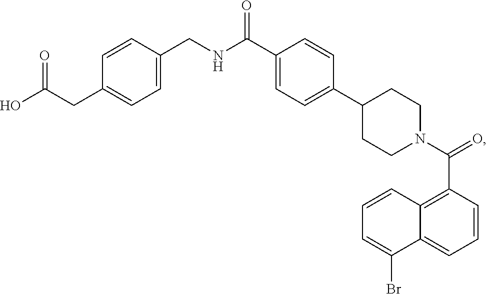 [SMILES: O=C(O)Cc1ccc(CNC(=O)c2ccc(C3CCN(C(=O)c4cccc5c(Br)cccc45)CC3)cc2)cc1] | (jo) |
| 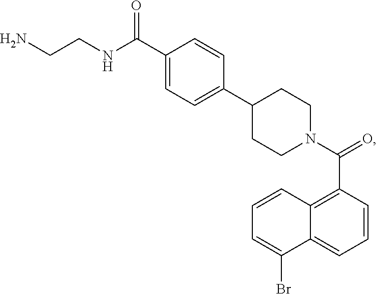 [SMILES: NCCNC(=O)c1ccc(C2CCN(C(=O)c3cccc4c(Br)cccc34)CC2)cc1] | (jp) |
| 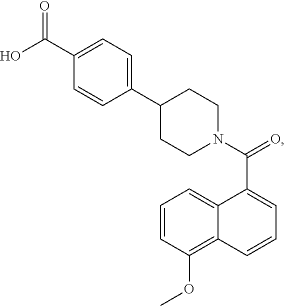 [SMILES: COc1cccc2c(C(=O)N3CCC(c4ccc(C(=O)O)cc4)CC3)cccc12] | (jq) |
| 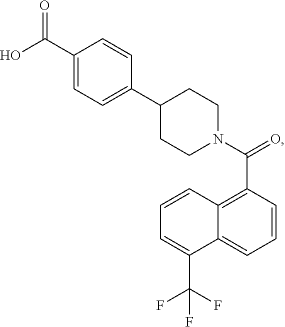 [SMILES: O=C(O)c1ccc(C2CCN(C(=O)c3cccc4c(C(F)(F)F)cccc34)CC2)cc1] | (jr) |
| 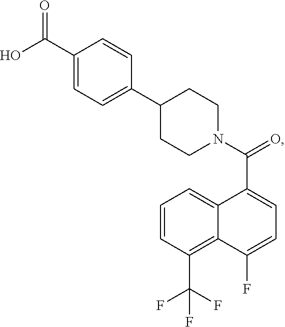 [SMILES: O=C(O)c1ccc(C2CCN(C(=O)c3ccc(F)c4c(C(F)(F)F)cccc34)CC2)cc1] | (js) |
| 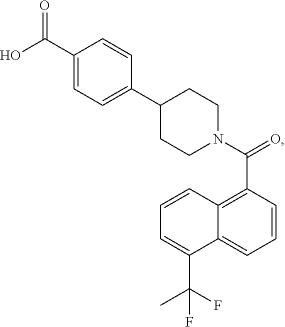 [SMILES: CC(F)(F)c1cccc2c(C(=O)N3CCC(c4ccc(C(=O)O)cc4)CC3)cccc12] | (jt) |
| 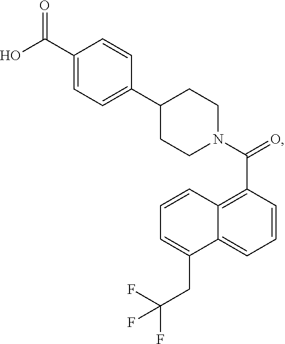 [SMILES: O=C(O)c1ccc(C2CCN(C(=O)c3cccc4c(CC(F)(F)F)cccc34)CC2)cc1] | (ju) |
| 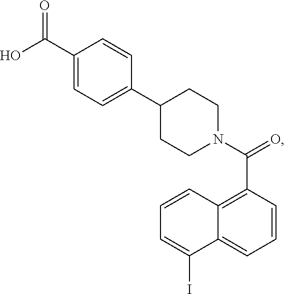 [SMILES: O=C(O)c1ccc(C2CCN(C(=O)c3cccc4c(I)cccc34)CC2)cc1] | (jv) |
| 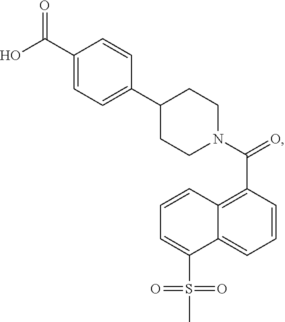 [SMILES: CS(=O)(=O)c1cccc2c(C(=O)N3CCC(c4ccc(C(=O)O)cc4)CC3)cccc12] | (jw) |
| 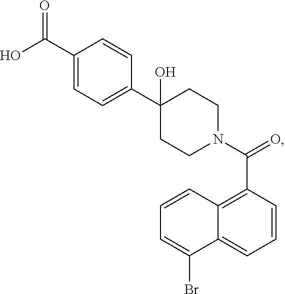 [SMILES: O=C(O)c1ccc(C2(O)CCN(C(=O)c3cccc4c(Br)cccc34)CC2)cc1] | (jx) |
| 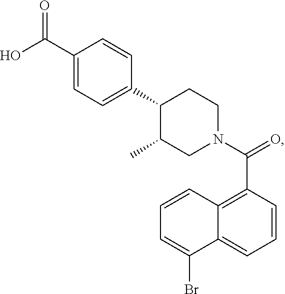 [SMILES: C[C@H]1CN(C(=O)c2cccc3c(Br)cccc23)CC[C@H]1c1ccc(C(=O)O)cc1] | (jy) |
| 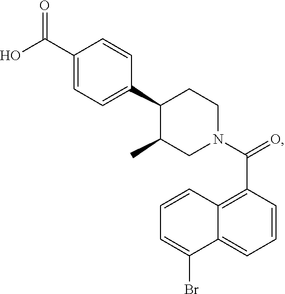 [SMILES: C[C@@H]1CN(C(=O)c2cccc3c(Br)cccc23)CC[C@@H]1c1ccc(C(=O)O)cc1] | (jz) |
| 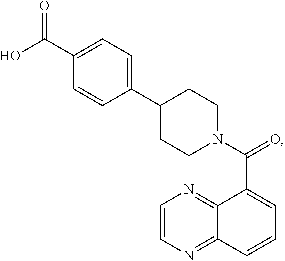 [SMILES: O=C(O)c1ccc(C2CCN(C(=O)c3cccc4nccnc34)CC2)cc1] | (ka) |
| 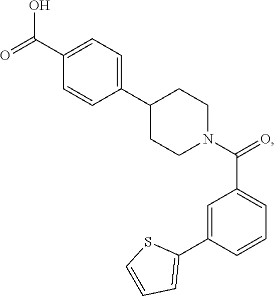 [SMILES: O=C(O)c1ccc(C2CCN(C(=O)c3cccc(-c4cccs4)c3)CC2)cc1] | (kb) |
| 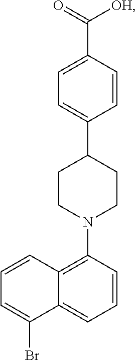 [SMILES: O=C(O)c1ccc(C2CCN(c3cccc4c(Br)cccc34)CC2)cc1] | (kc) |
| 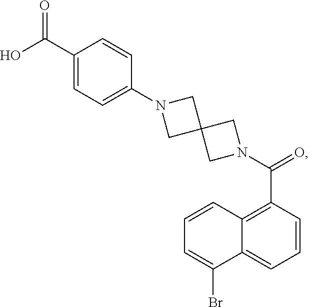 [SMILES: O=C(O)c1ccc(N2CC3(CN(C(=O)c4cccc5c(Br)cccc45)C3)C2)cc1] | (kd) |
| 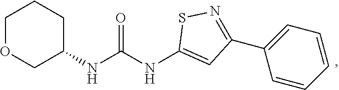 [SMILES: O=C(Nc1cc(-c2ccccc2)ns1)N[C@H]1CCCOC1] | (kw) |
| 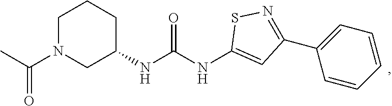 [SMILES: CC(=O)N1CCC[C@H](NC(=O)Nc2cc(-c3ccccc3)ns2)C1] | (kx) |
| 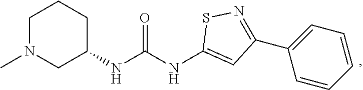 [SMILES: CN1CCC[C@H](NC(=O)Nc2cc(-c3ccccc3)ns2)C1] | (ky) |
| 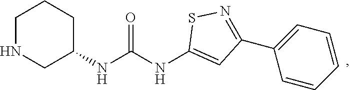 [SMILES: O=C(Nc1cc(-c2ccccc2)ns1)N[C@H]1CCCNC1] | (kz) |
| 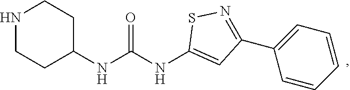 [SMILES: O=C(Nc1cc(-c2ccccc2)ns1)NC1CCNCC1] | (la) |
| 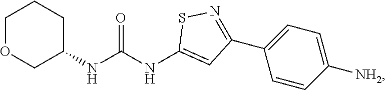 [SMILES: Nc1ccc(-c2cc(NC(=O)N[C@H]3CCCOC3)sn2)cc1] | (lb) |
| 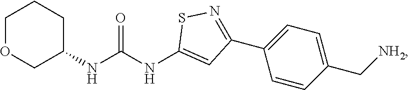 [SMILES: NCc1ccc(-c2cc(NC(=O)N[C@H]3CCCOC3)sn2)cc1] | (lc) |
| 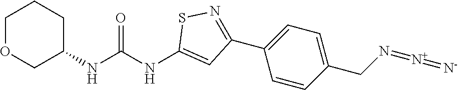 [SMILES: [N-]=[N+]=NCc1ccc(-c2cc(NC(=O)N[C@H]3CCCOC3)sn2)cc1] | (ld) |
| 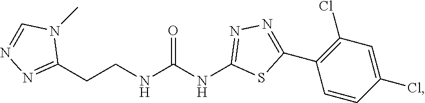 [SMILES: Cn1cnnc1CCNC(=O)Nc1nnc(-c2ccc(Cl)cc2Cl)s1] | (le) |
| 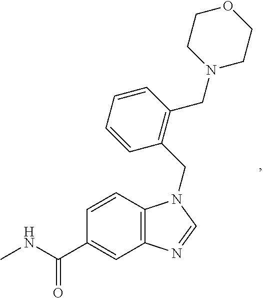 [SMILES: CNC(=O)c1ccc2c(c1)ncn2Cc1ccccc1CN1CCOCC1] | (lf) |
| 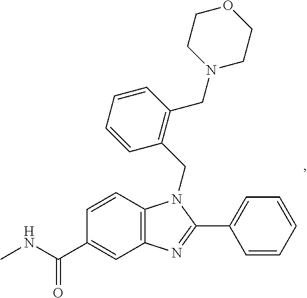 [SMILES: CNC(=O)c1ccc2c(c1)nc(-c1ccccc1)n2Cc1ccccc1CN1CCOCC1] | (lg) |
| 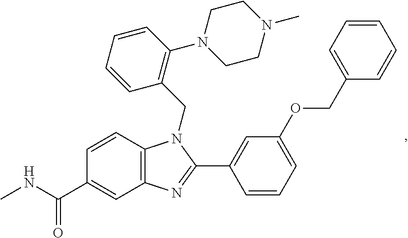 [SMILES: CNC(=O)c1ccc2c(c1)nc(-c1cccc(OCc3ccccc3)c1)n2Cc1ccccc1N1CCN(C)CC1] | (lh) |
| 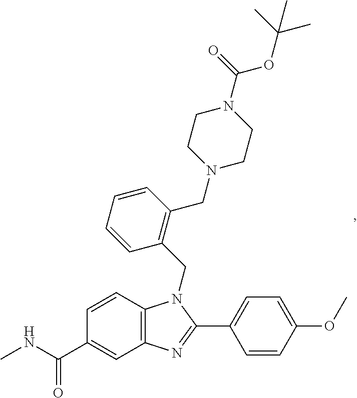 | (li) |
| 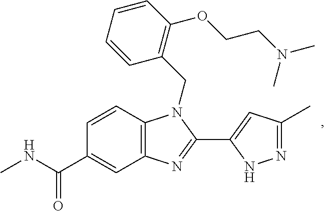 | (lj) |
| 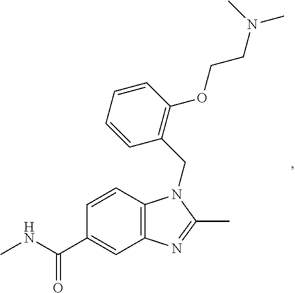 | (lk) |
| 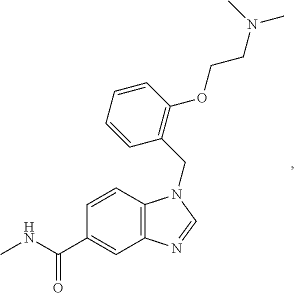 | (ll) |
| 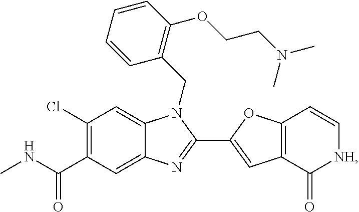 [SMILES: CNC(=O)c1cc2nc(-c3cc4c(=O)[nH]ccc4o3)n(Cc3ccccc3OCCN(C)C)c2cc1Cl] | (lm) |
| 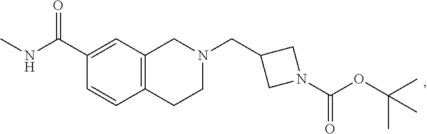 [SMILES: CNC(=O)c1ccc2c(c1)CN(CC1CN(C(=O)OC(C)(C)C)C1)CC2] | (ln) |
| 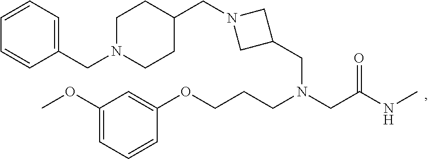 [SMILES: CNC(=O)CN(CCCOc1cccc(OC)c1)CC1CN(CC2CCN(Cc3ccccc3)CC2)C1] | (lo) |
| 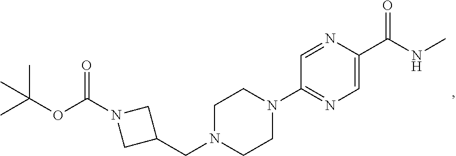 [SMILES: CNC(=O)c1cnc(N2CCN(CC3CN(C(=O)OC(C)(C)C)C3)CC2)cn1] | (lp) |
| 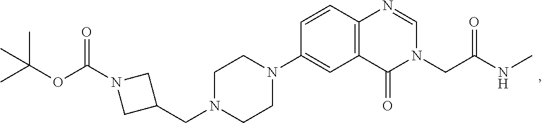 [SMILES: CNC(=O)Cn1cnc2ccc(N3CCN(CC4CN(C(=O)OC(C)(C)C)C4)CC3)cc2c1=O] | (lq) |
| 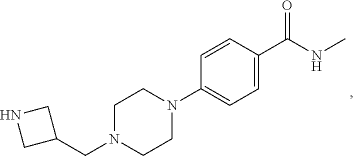 [SMILES: CNC(=O)c1ccc(N2CCN(CC3CNC3)CC2)cc1] | (lr) |
| 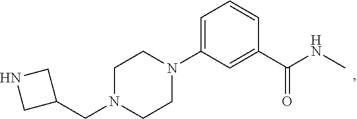 [SMILES: CNC(=O)c1cccc(N2CCN(CC3CNC3)CC2)c1] | (ls) |
| 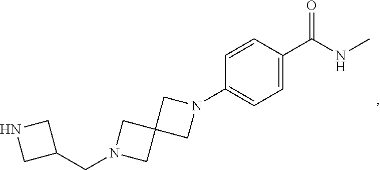 [SMILES: CNC(=O)c1ccc(N2CC3(CN(CC4CNC4)C3)C2)cc1] | (lt) |
| 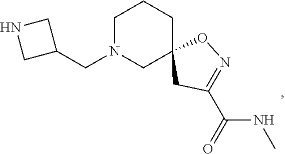 [SMILES: CNC(=O)C1=NO[C@]2(CCCN(CC3CNC3)C2)C1] | (lu) |
| 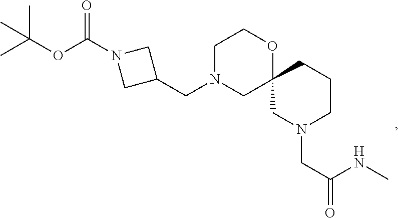 [SMILES: CNC(=O)CN1CCC[C@@]2(C1)CN(CC1CN(C(=O)OC(C)(C)C)C1)CCO2] | (lv) |
| 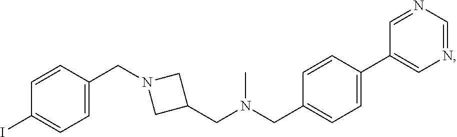 [SMILES: CN(Cc1ccc(-c2cncnc2)cc1)CC1CN(Cc2ccc(I)cc2)C1] | (lw) |
| 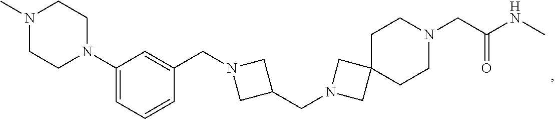 | (lx) |
| 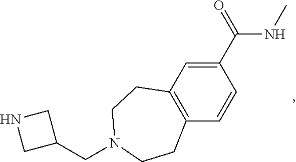 [SMILES: CNC(=O)c1ccc2c(c1)CCN(CC1CNC1)CC2] | (ly) |
| 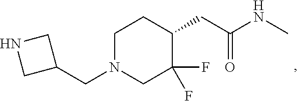 [SMILES: CNC(=O)C[C@H]1CCN(CC2CNC2)CC1(F)F] | (lz) |
| 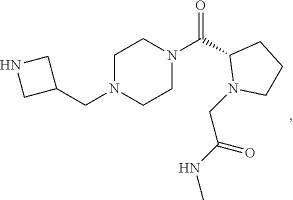 [SMILES: CNC(=O)CN1CCC[C@H]1C(=O)N1CCN(CC2CNC2)CC1] | (ma) |
| 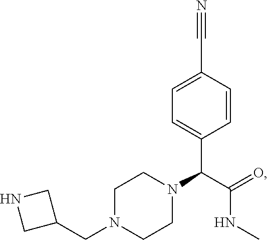 [SMILES: CNC(=O)[C@H](c1ccc(C#N)cc1)N1CCN(CC2CNC2)CC1] | (mb) |
| 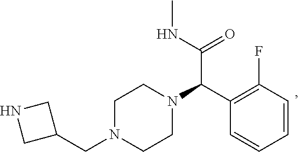 [SMILES: CNC(=O)[C@@H](c1ccccc1F)N1CCN(CC2CNC2)CC1] | (mc) |
| 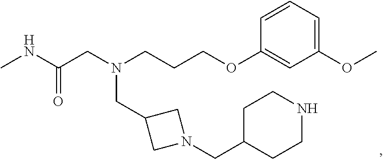 | (md) |
| 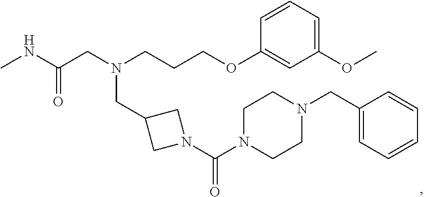 [SMILES: CNC(=O)CN(CCCOc1cccc(OC)c1)CC1CN(C(=O)N2CCN(Cc3ccccc3)CC2)C1] | (me) |
| 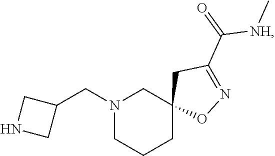 [SMILES: CNC(=O)C1=NO[C@@]2(CCCN(CC3CNC3)C2)C1] | (mf) |
| 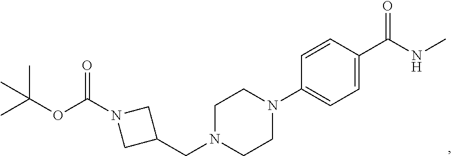 [SMILES: CNC(=O)c1ccc(N2CCN(CC3CN(C(=O)OC(C)(C)C)C3)CC2)cc1] | (mg) |
| 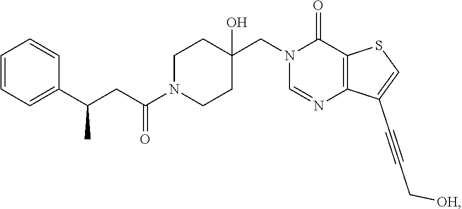 [SMILES: C[C@H](CC(=O)N1CCC(O)(Cn2cnc3c(C#CCO)csc3c2=O)CC1)c1ccccc1] | (mh) |
| 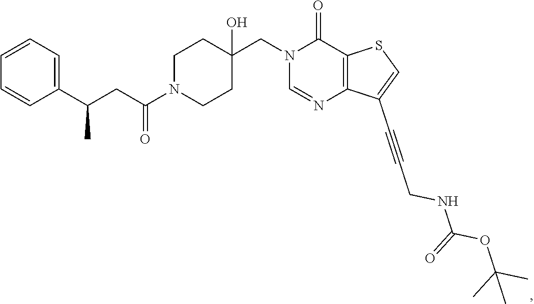 [SMILES: C[C@H](CC(=O)N1CCC(O)(Cn2cnc3c(C#CCNC(=O)OC(C)(C)C)csc3c2=O)CC1)c1ccccc1] | (mi) |
| 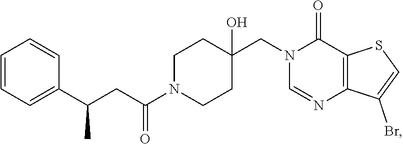 [SMILES: C[C@H](CC(=O)N1CCC(O)(Cn2cnc3c(Br)csc3c2=O)CC1)c1ccccc1] | (mj) |
| 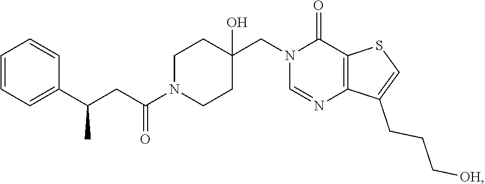 [SMILES: C[C@H](CC(=O)N1CCC(O)(Cn2cnc3c(CCCO)csc3c2=O)CC1)c1ccccc1] | (mk) |
| 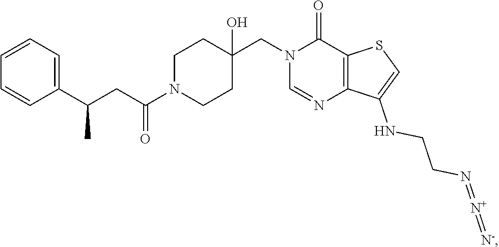 | (ml) |
| 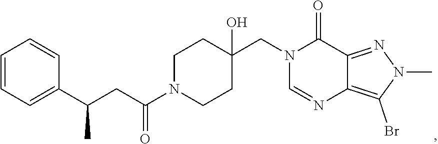 [SMILES: C[C@H](CC(=O)N1CCC(O)(Cn2cnc3c(Br)n(C)nc3c2=O)CC1)c1ccccc1] | (mm) |
| 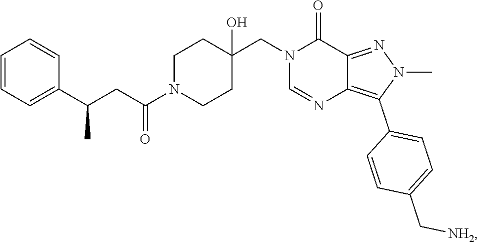 [SMILES: C[C@H](CC(=O)N1CCC(O)(Cn2cnc3c(-c4ccc(CN)cc4)n(C)nc3c2=O)CC1)c1ccccc1] | (mn) |
| 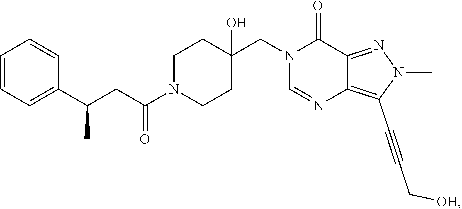 [SMILES: C[C@H](CC(=O)N1CCC(O)(Cn2cnc3c(C#CCO)n(C)nc3c2=O)CC1)c1ccccc1] | (mo) |
| 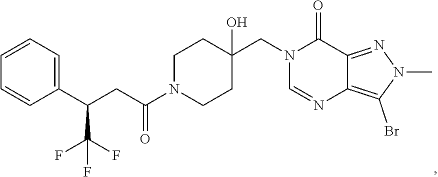 [SMILES: Cn1nc2c(=O)n(CC3(O)CCN(C(=O)C[C@H](c4ccccc4)C(F)(F)F)CC3)cnc2c1Br] | (mp) |
| 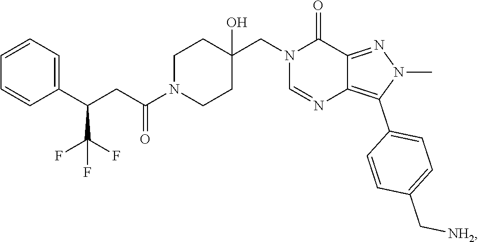 [SMILES: Cn1nc2c(=O)n(CC3(O)CCN(C(=O)C[C@H](c4ccccc4)C(F)(F)F)CC3)cnc2c1-c1ccc(CN)cc1] | (mq) |
| 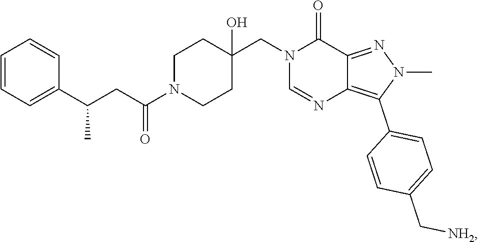 [SMILES: C[C@@H](CC(=O)N1CCC(O)(Cn2cnc3c(-c4ccc(CN)cc4)n(C)nc3c2=O)CC1)c1ccccc1] | (mr) |
| 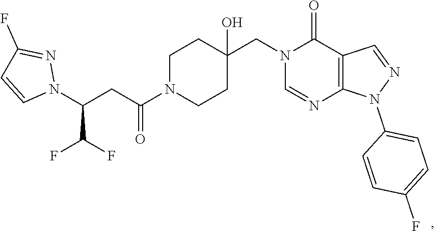 [SMILES: O=C(C[C@@H](C(F)F)n1ccc(F)n1)N1CCC(O)(Cn2cnc3c(cnn3-c3ccc(F)cc3)c2=O)CC1] | (ms) |
| 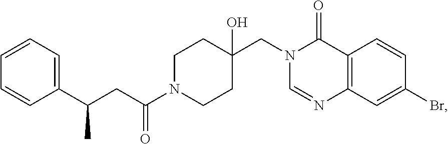 [SMILES: C[C@H](CC(=O)N1CCC(O)(Cn2cnc3cc(Br)ccc3c2=O)CC1)c1ccccc1] | (mt) |
| 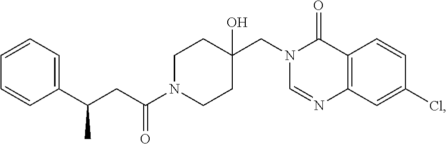 [SMILES: C[C@H](CC(=O)N1CCC(O)(Cn2cnc3cc(Cl)ccc3c2=O)CC1)c1ccccc1] | (mu) |
| 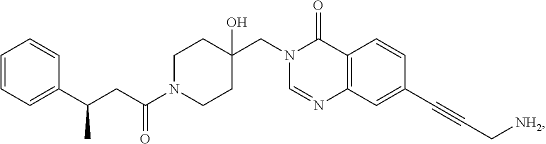 [SMILES: C[C@H](CC(=O)N1CCC(O)(Cn2cnc3cc(C#CCN)ccc3c2=O)CC1)c1ccccc1] | (mv) |
| 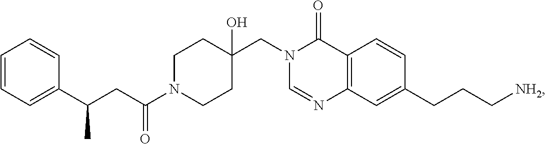 [SMILES: C[C@H](CC(=O)N1CCC(O)(Cn2cnc3cc(CCCN)ccc3c2=O)CC1)c1ccccc1] | (mw) |
| 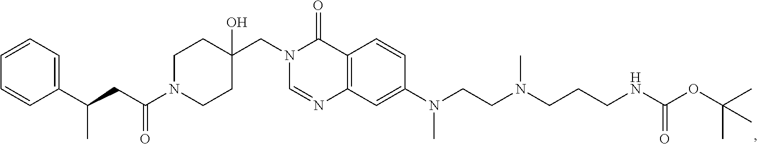 [SMILES: C[C@H](CC(=O)N1CCC(O)(Cn2cnc3cc(N(C)CCN(C)CCCNC(=O)OC(C)(C)C)ccc3c2=O)CC1)c1ccccc1] | (mx) |
| 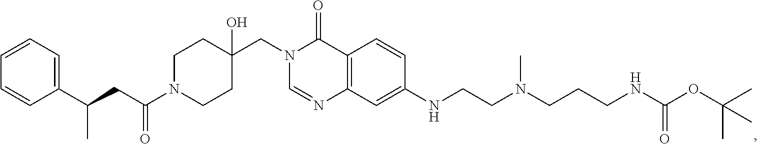 [SMILES: C[C@H](CC(=O)N1CCC(O)(Cn2cnc3cc(NCCN(C)CCCNC(=O)OC(C)(C)C)ccc3c2=O)CC1)c1ccccc1] | (my) |
| 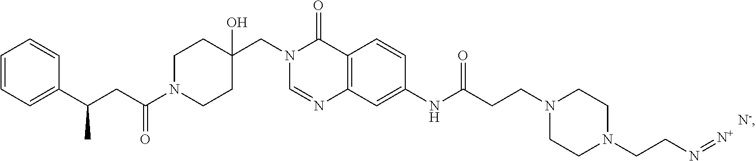 [SMILES: C[C@H](CC(=O)N1CCC(O)(Cn2cnc3cc(NC(=O)CCN4CCN(CCN=[NH2+])CC4)ccc3c2=O)CC1)c1ccccc1] | (mz) |
| 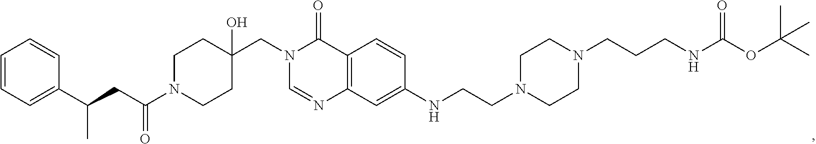 [SMILES: C[C@H](CC(=O)N1CCC(O)(Cn2cnc3cc(NCCN4CCN(CCCNC(=O)OC(C)(C)C)CC4)ccc3c2=O)CC1)c1ccccc1] | (na) |
| 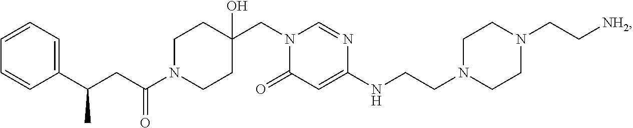 [SMILES: C[C@H](CC(=O)N1CCC(O)(Cn2cnc(NCCN3CCN(CCN)CC3)cc2=O)CC1)c1ccccc1] | (nb) |
| 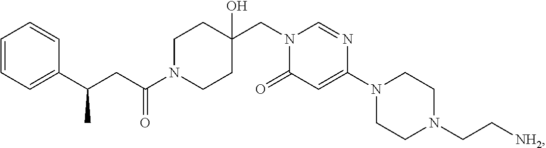 [SMILES: C[C@H](CC(=O)N1CCC(O)(Cn2cnc(N3CCN(CCN)CC3)cc2=O)CC1)c1ccccc1] | (nc) |
| 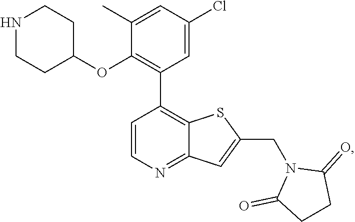 [SMILES: Cc1cc(Cl)cc(-c2ccnc3cc(CN4C(=O)CCC4=O)sc23)c1OC1CCNCC1] | (nd) |
| 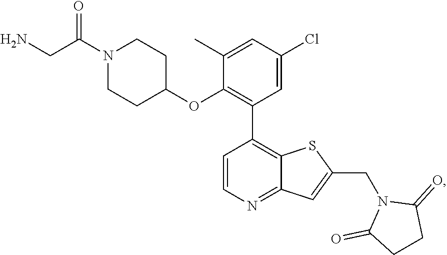 [SMILES: Cc1cc(Cl)cc(-c2ccnc3cc(CN4C(=O)CCC4=O)sc23)c1OC1CCN(C(=O)CN)CC1] | (ne) |
| 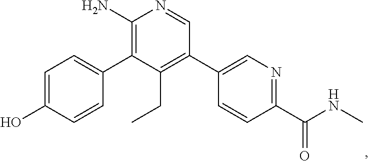 [SMILES: CCc1c(-c2ccc(C(=O)NC)nc2)cnc(N)c1-c1ccc(O)cc1] | (nf) |
| 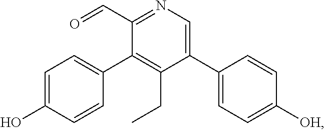 [SMILES: CCc1c(-c2ccc(O)cc2)cnc(C=O)c1-c1ccc(O)cc1] | (ng) |
| 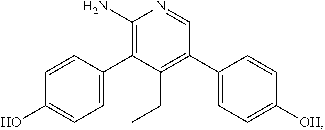 [SMILES: CCc1c(-c2ccc(O)cc2)cnc(N)c1-c1ccc(O)cc1] | (nh) |
| 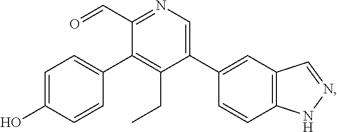 [SMILES: CCc1c(-c2ccc3[nH]ncc3c2)cnc(C=O)c1-c1ccc(O)cc1] | (ni) |
| 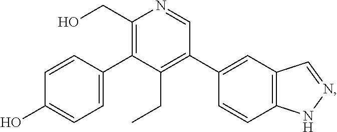 [SMILES: CCc1c(-c2ccc3[nH]ncc3c2)cnc(CO)c1-c1ccc(O)cc1] | (nj) |
| 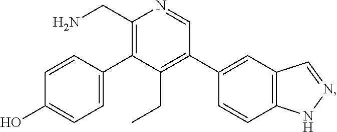 [SMILES: CCc1c(-c2ccc3[nH]ncc3c2)cnc(CN)c1-c1ccc(O)cc1] | (nk) |
| 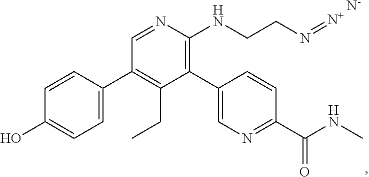 | (nl) |
| 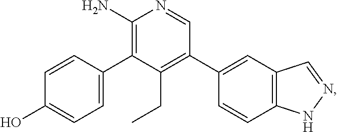 [SMILES: CCc1c(-c2ccc3[nH]ncc3c2)cnc(N)c1-c1ccc(O)cc1] | (nm) |
| 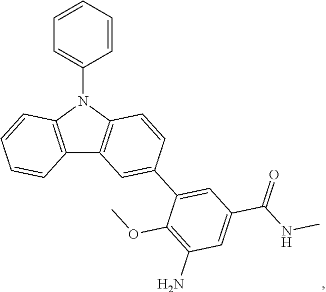 [SMILES: CNC(=O)c1cc(N)c(OC)c(-c2ccc3c(c2)c2ccccc2n3-c2ccccc2)c1] | (nn) |
| 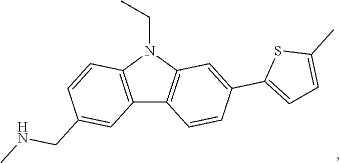 | (no) |
| 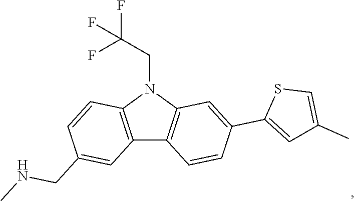 | (np) |
| 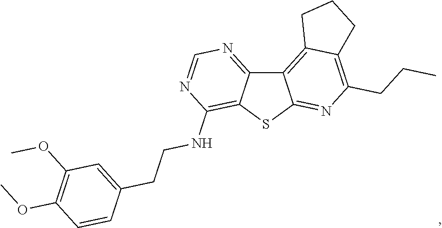 [SMILES: CCCc1nc2sc3c(NCCc4ccc(OC)c(OC)c4)ncnc3c2c2c1CCC2] | (nq) |
| 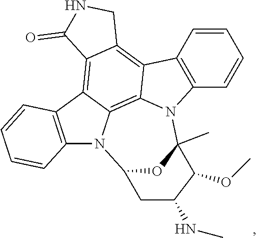 [SMILES: CN[C@@H]1C[C@H]2O[C@@](C)([C@@H]1OC)n1c3ccccc3c3c4c(c5c6ccccc6n2c5c31)C(=O)NC4] | (nr) |
| 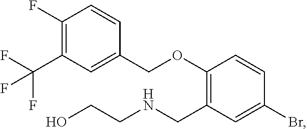 [SMILES: OCCNCc1cc(Br)ccc1OCc1ccc(F)c(C(F)(F)F)c1] | (ns) |
| 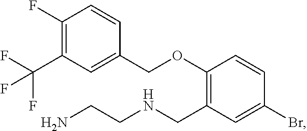 [SMILES: NCCNCc1cc(Br)ccc1OCc1ccc(F)c(C(F)(F)F)c1] | (nt) |
| 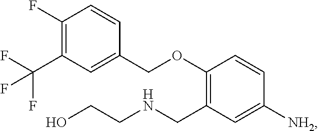 [SMILES: Nc1ccc(OCc2ccc(F)c(C(F)(F)F)c2)c(CNCCO)c1] | (nu) |
| 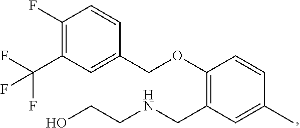 [SMILES: Cc1ccc(OCc2ccc(F)c(C(F)(F)F)c2)c(CNCCO)c1] | (nv) |
| 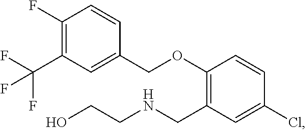 [SMILES: OCCNCc1cc(Cl)ccc1OCc1ccc(F)c(C(F)(F)F)c1] | (nw) |
| 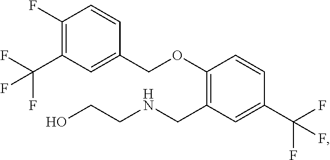 [SMILES: OCCNCc1cc(C(F)(F)F)ccc1OCc1ccc(F)c(C(F)(F)F)c1] | (nx) |
| 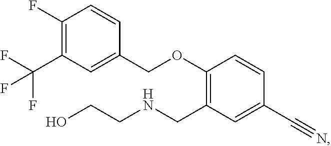 [SMILES: N#Cc1ccc(OCc2ccc(F)c(C(F)(F)F)c2)c(CNCCO)c1] | (ny) |
| 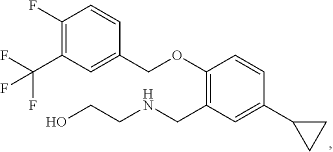 [SMILES: OCCNCc1cc(C2CC2)ccc1OCc1ccc(F)c(C(F)(F)F)c1] | (nz) |
| 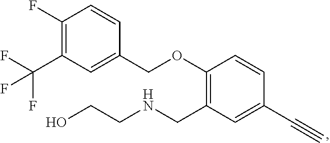 [SMILES: C#Cc1ccc(OCc2ccc(F)c(C(F)(F)F)c2)c(CNCCO)c1] | (oa) |
| 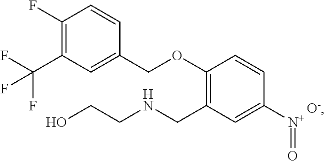 | (ob) |
| 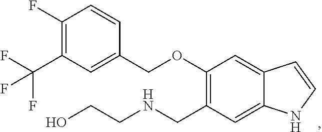 [SMILES: OCCNCc1cc2[nH]ccc2cc1OCc1ccc(F)c(C(F)(F)F)c1] | (oc) |
| 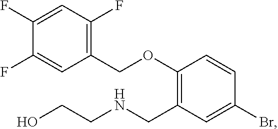 [SMILES: OCCNCc1cc(Br)ccc1OCc1cc(F)c(F)cc1F] | (od) |
| 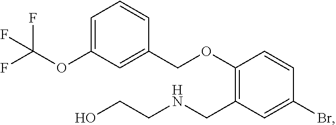 [SMILES: OCCNCc1cc(Br)ccc1OCc1cccc(OC(F)(F)F)c1] | (oe) |
| 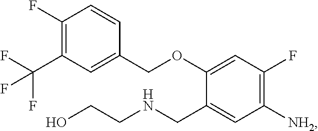 [SMILES: Nc1cc(CNCCO)c(OCc2ccc(F)c(C(F)(F)F)c2)cc1F] | (of) |
| 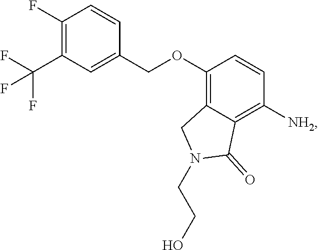 [SMILES: Nc1ccc(OCc2ccc(F)c(C(F)(F)F)c2)c2c1C(=O)N(CCO)C2] | (og) |
| 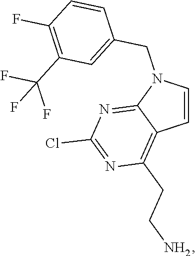 [SMILES: NCCc1nc(Cl)nc2c1ccn2Cc1ccc(F)c(C(F)(F)F)c1] | (oh) |
| 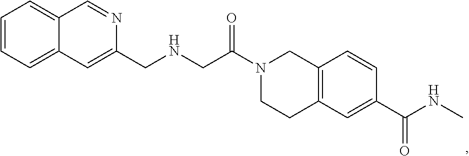 [SMILES: CNC(=O)c1ccc2c(c1)CCN(C(=O)CNCc1cc3ccccc3cn1)C2] | (oi) |
| 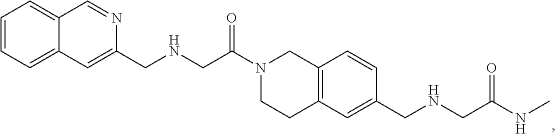 [SMILES: CNC(=O)CNCc1ccc2c(c1)CCN(C(=O)CNCc1cc3ccccc3cn1)C2] | (oj) |
| 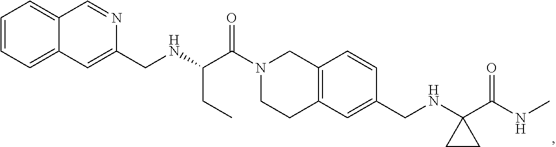 [SMILES: CC[C@H](NCc1cc2ccccc2cn1)C(=O)N1CCc2cc(CNC3(C(=O)NC)CC3)ccc2C1] | (ok) |
| 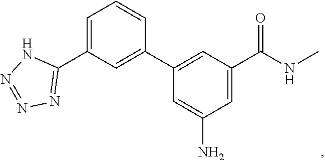 [SMILES: CNC(=O)c1cc(N)cc(-c2cccc(-c3nnn[nH]3)c2)c1] | (ol) |
| 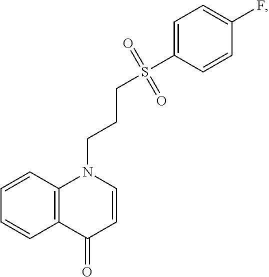 [SMILES: O=c1ccn(CCCS(=O)(=O)c2ccc(F)cc2)c2ccccc12] | (om) |
| 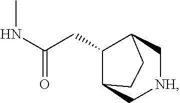 | (on) |
| 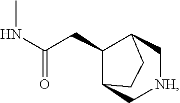 | (oo) |
| 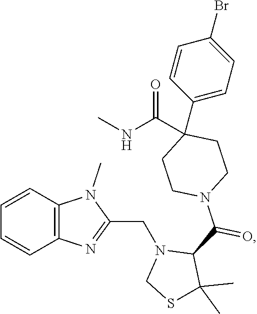 [SMILES: CNC(=O)C1(c2ccc(Br)cc2)CCN(C(=O)[C@@H]2N(Cc3nc4ccccc4n3C)CSC2(C)C)CC1] | (op) |
| 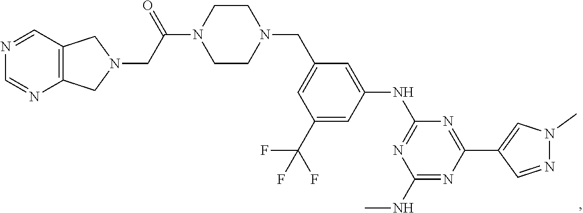 [SMILES: CNc1nc(Nc2cc(CN3CCN(C(=O)CN4Cc5cncnc5C4)CC3)cc(C(F)(F)F)c2)nc(-c2cnn(C)c2)n1] | (oq) |
| 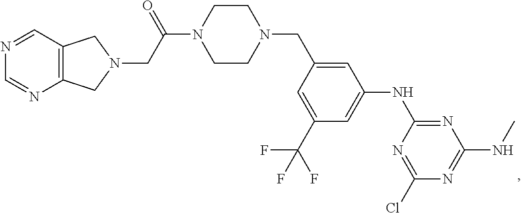 [SMILES: CNc1nc(Cl)nc(Nc2cc(CN3CCN(C(=O)CN4Cc5cncnc5C4)CC3)cc(C(F)(F)F)c2)n1] | (or) |
| 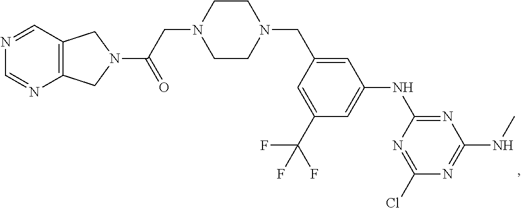 [SMILES: CNc1nc(Cl)nc(Nc2cc(CN3CCN(CC(=O)N4Cc5cncnc5C4)CC3)cc(C(F)(F)F)c2)n1] | (os) |
| 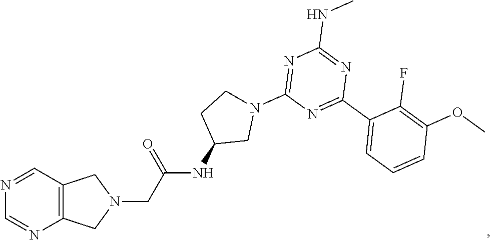 [SMILES: CNc1nc(-c2cccc(OC)c2F)nc(N2CC[C@H](NC(=O)CN3Cc4cncnc4C3)C2)n1] | (ot) |
| 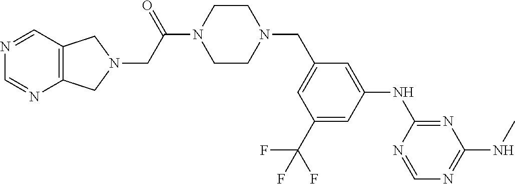 [SMILES: CNc1ncnc(Nc2cc(CN3CCN(C(=O)CN4Cc5cncnc5C4)CC3)cc(C(F)(F)F)c2)n1] | (ou) |
| 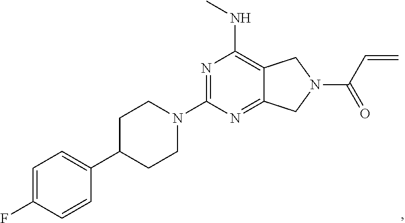 [SMILES: C=CC(=O)N1Cc2nc(N3CCC(c4ccc(F)cc4)CC3)nc(NC)c2C1] | (ov) |
| 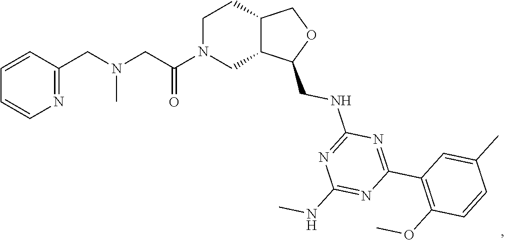 [SMILES: CNc1nc(NC[C@@H]2OC[C@H]3CCN(C(=O)CN(C)Cc4ccccn4)C[C@H]32)nc(-c2cc(C)ccc2OC)n1] | (ow) |
| 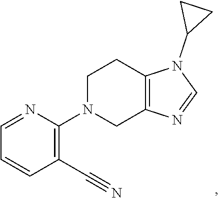 [SMILES: N#Cc1cccnc1N1CCc2c(ncn2C2CC2)C1] | (ox) |
| 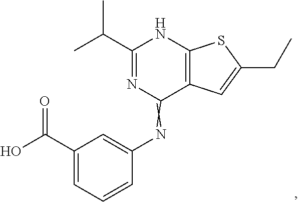 [SMILES: CCc1cc2c(=Nc3cccc(C(=O)O)c3)nc(C(C)C)[nH]c2s1] | (oy) |
| 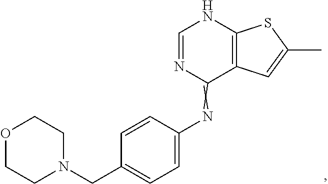 [SMILES: Cc1cc2c(=Nc3ccc(CN4CCOCC4)cc3)nc[nH]c2s1] | (oz) |
| 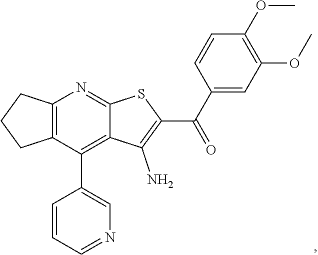 [SMILES: COc1ccc(C(=O)c2sc3nc4c(c(-c5cccnc5)c3c2N)CCC4)cc1OC] | (pa) |
| 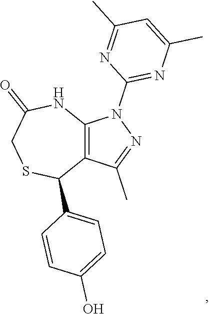 [SMILES: Cc1cc(C)nc(-n2nc(C)c3c2NC(=O)CS[C@@H]3c2ccc(O)cc2)n1] | (pb) |
| 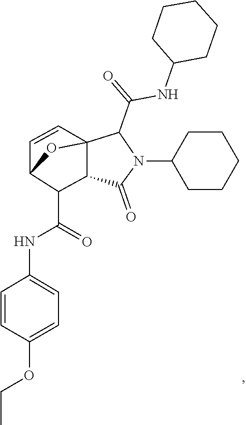 | (pc) |
| 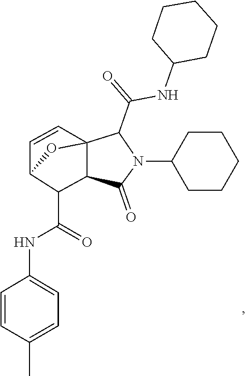 | (pd) |
| 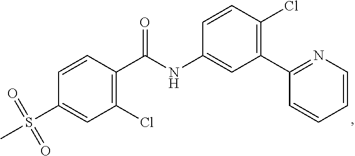 [SMILES: CS(=O)(=O)c1ccc(C(=O)Nc2ccc(Cl)c(-c3ccccn3)c2)c(Cl)c1] | (pe) |
| 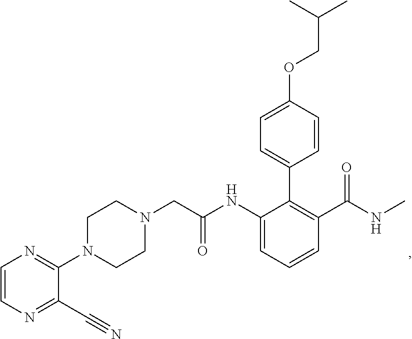 [SMILES: CNC(=O)c1cccc(NC(=O)CN2CCN(c3nccnc3C#N)CC2)c1-c1ccc(OCC(C)C)cc1] | (pf) |
| 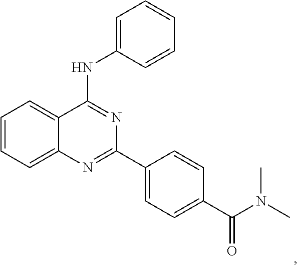 [SMILES: CN(C)C(=O)c1ccc(-c2nc(Nc3ccccc3)c3ccccc3n2)cc1] | (pg) |
| 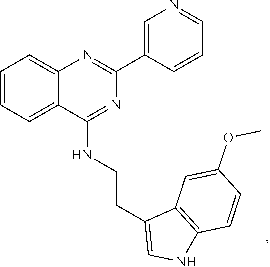 [SMILES: COc1ccc2[nH]cc(CCNc3nc(-c4cccnc4)nc4ccccc34)c2c1] | (ph) |
| 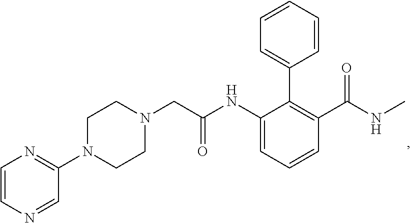 [SMILES: CNC(=O)c1cccc(NC(=O)CN2CCN(c3cnccn3)CC2)c1-c1ccccc1] | (pi) |
| 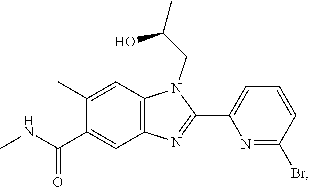 [SMILES: CNC(=O)c1cc2nc(-c3cccc(Br)n3)n(C[C@H](C)O)c2cc1C] | (pj) |
| 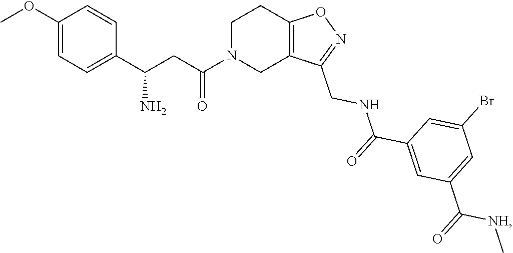 | (pk) |
| 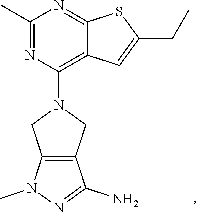 [SMILES: CCc1cc2c(N3Cc4c(N)nn(C)c4C3)nc(C)nc2s1] | (pl) |
| 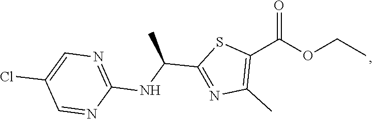 [SMILES: CCOC(=O)c1sc([C@H](C)Nc2ncc(Cl)cn2)nc1C] | (pm) |
| 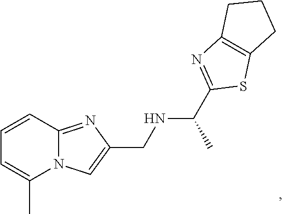 [SMILES: Cc1cccc2nc(CN[C@@H](C)c3nc4c(s3)CCC4)cn12] | (pn) |
| 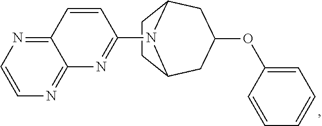 [SMILES: c1ccc(OC2CC3CCC(C2)N3c2ccc3nccnc3n2)cc1] | (po) |
| 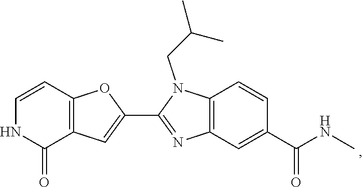 [SMILES: CNC(=O)c1ccc2c(c1)nc(-c1cc3c(=O)[nH]ccc3o1)n2CC(C)C] | (pp) |
| 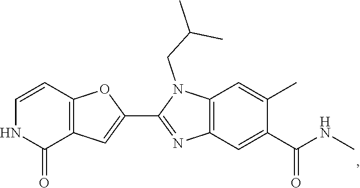 [SMILES: CNC(=O)c1cc2nc(-c3cc4c(=O)[nH]ccc4o3)n(CC(C)C)c2cc1C] | (pq) |
| 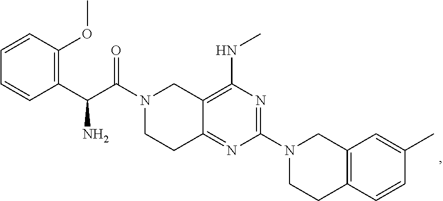 [SMILES: CNc1nc(N2CCc3ccc(C)cc3C2)nc2c1CN(C(=O)[C@@H](N)c1ccccc1OC)CC2] | (pr) |
| 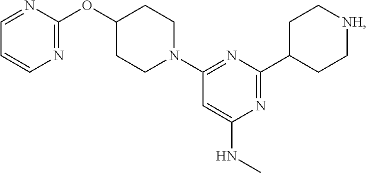 [SMILES: CNc1cc(N2CCC(Oc3ncccn3)CC2)nc(C2CCNCC2)n1] | (ps) |
| 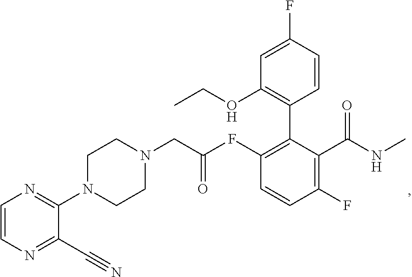 | (pt) |
| 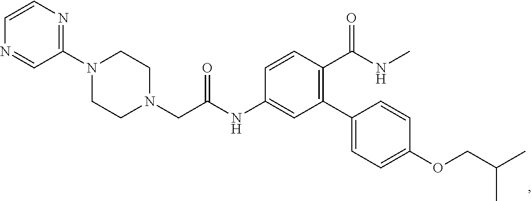 [SMILES: CNC(=O)c1ccc(NC(=O)CN2CCN(c3cnccn3)CC2)cc1-c1ccc(OCC(C)C)cc1] | (pu) |
| 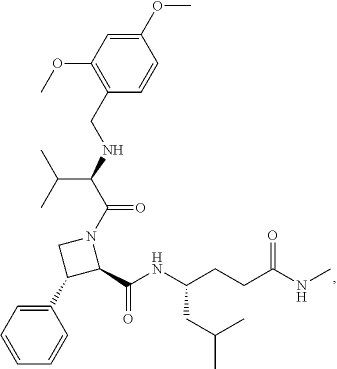 [SMILES: CNC(=O)CC[C@H](CC(C)C)NC(=O)[C@H]1[C@H](c2ccccc2)CN1C(=O)[C@H](NCc1ccc(OC)cc1OC)C(C)C] | (pv) |
| 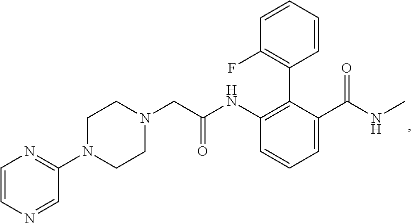 [SMILES: CNC(=O)c1cccc(NC(=O)CN2CCN(c3cnccn3)CC2)c1-c1ccccc1F] | (pw) |
| 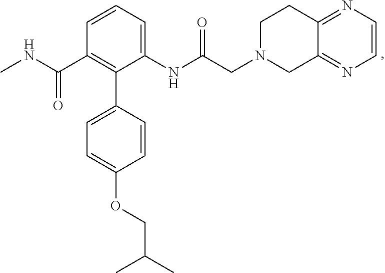 [SMILES: CNC(=O)c1cccc(NC(=O)CN2CCc3nccnc3C2)c1-c1ccc(OCC(C)C)cc1] | (px) |
| 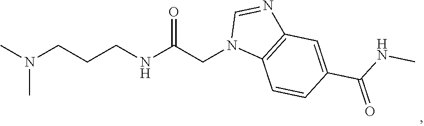 | (py) |
| 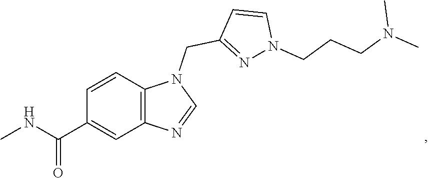 | (pz) |
| 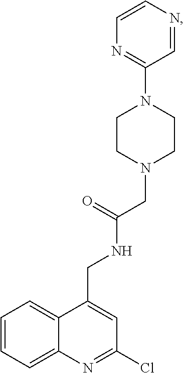 [SMILES: O=C(CN1CCN(c2cnccn2)CC1)NCc1cc(Cl)nc2ccccc12] | (qa) |
| 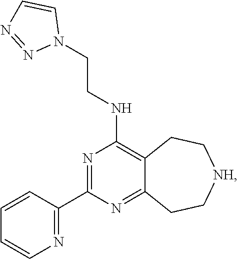 [SMILES: c1ccc(-c2nc3c(c(NCCn4ccnn4)n2)CCNCC3)nc1] | (qb) |
| 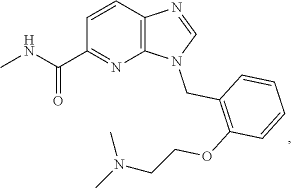 [SMILES: CNC(=O)c1ccc2ncn(Cc3ccccc3OCCN(C)C)c2n1] | (qc) |
| 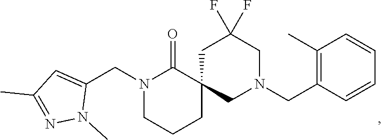 [SMILES: Cc1cc(CN2CCC[C@]3(CN(Cc4ccccc4C)CC(F)(F)C3)C2=O)n(C)n1] | (qd) |
| 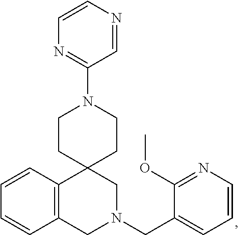 [SMILES: COc1ncccc1CN1Cc2ccccc2C2(CCN(c3cnccn3)CC2)C1] | (qe) |
| 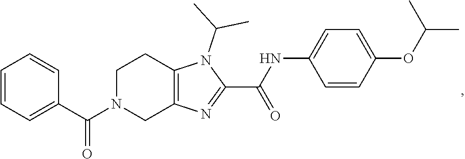 [SMILES: CC(C)Oc1ccc(NC(=O)c2nc3c(n2C(C)C)CCN(C(=O)c2ccccc2)C3)cc1] | (qf) |
| 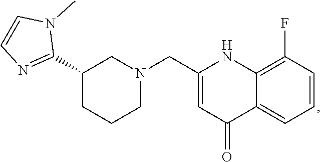 [SMILES: Cn1ccnc1[C@H]1CCCN(Cc2cc(=O)c3cccc(F)c3[nH]2)C1] | (qg) |
| 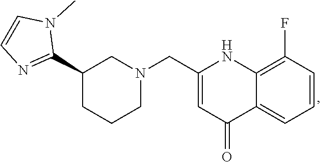 [SMILES: Cn1ccnc1[C@@H]1CCCN(Cc2cc(=O)c3cccc(F)c3[nH]2)C1] | (qh) |
| 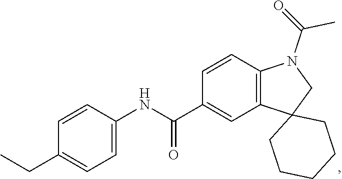 [SMILES: CCc1ccc(NC(=O)c2ccc3c(c2)C2(CCCCC2)CN3C(C)=O)cc1] | (qi) |
| 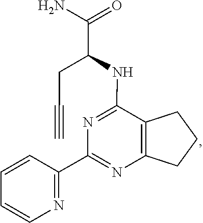 [SMILES: C#CC[C@H](Nc1nc(-c2ccccn2)nc2c1CCC2)C(N)=O] | (qj) |
| 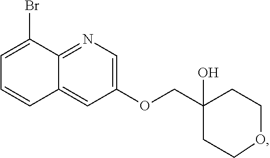 [SMILES: OC1(COc2cnc3c(Br)cccc3c2)CCOCC1] | (qk) |
| 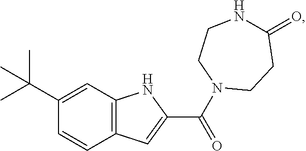 [SMILES: CC(C)(C)c1ccc2cc(C(=O)N3CCNC(=O)CC3)[nH]c2c1] | (ql) |
| 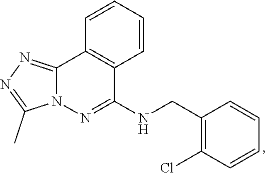 [SMILES: Cc1nnc2c3ccccc3c(NCc3ccccc3Cl)nn12] | (qm) |
| 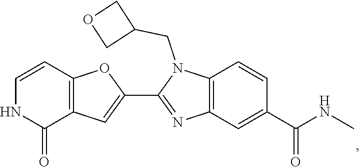 [SMILES: CNC(=O)c1ccc2c(c1)nc(-c1cc3c(=O)[nH]ccc3o1)n2CC1COC1] | (qn) |
| 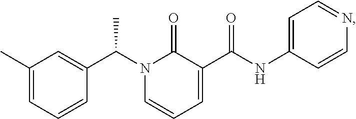 [SMILES: Cc1cccc([C@H](C)n2cccc(C(=O)Nc3ccncc3)c2=O)c1] | (qo) |
| 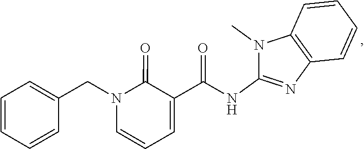 [SMILES: Cn1c(NC(=O)c2cccn(Cc3ccccc3)c2=O)nc2ccccc21] | (qp) |
| 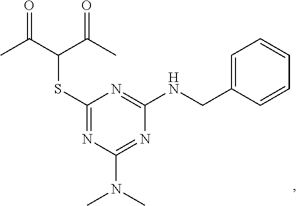 [SMILES: CC(=O)C(Sc1nc(NCc2ccccc2)nc(N(C)C)n1)C(C)=O] | (qq) |
| 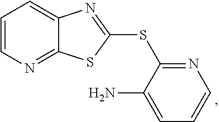 [SMILES: Nc1cccnc1Sc1nc2cccnc2s1] | (qr) |
| 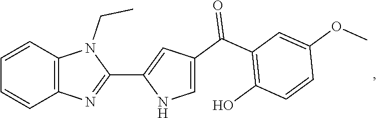 [SMILES: CCn1c(-c2cc(C(=O)c3cc(OC)ccc3O)c[nH]2)nc2ccccc21] | (qs) |
| 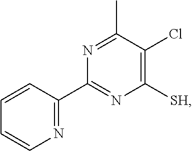 | (qt) |
| 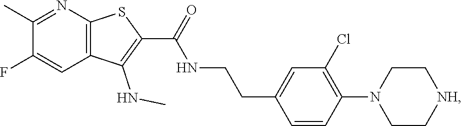 [SMILES: CNc1c(C(=O)NCCc2ccc(N3CCNCC3)c(Cl)c2)sc2nc(C)c(F)cc12] | (qu) |
| 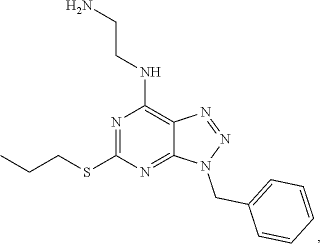 [SMILES: CCCSc1nc(NCCN)c2nnn(Cc3ccccc3)c2n1] | (qv) |
| 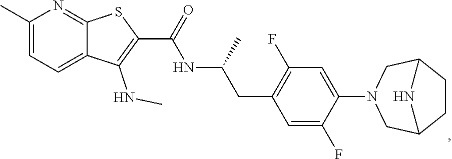 [SMILES: CNc1c(C(=O)N[C@H](C)Cc2cc(F)c(N3CC4CCC(C3)N4)cc2F)sc2nc(C)ccc12] | (qw) |
| 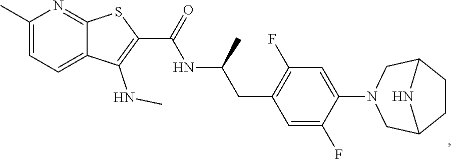 [SMILES: CNc1c(C(=O)N[C@@H](C)Cc2cc(F)c(N3CC4CCC(C3)N4)cc2F)sc2nc(C)ccc12] | (qx) |
| 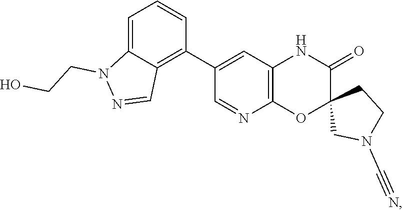 | (qy) |
| 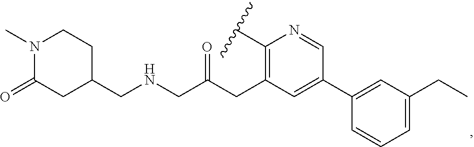 [SMILES: *c1ncc(-c2cccc(CC)c2)cc1CC(=O)CNCC1CCN(C)C(=O)C1] | (qw) |
| 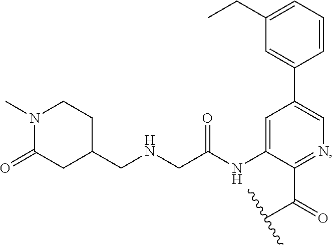 [SMILES: *C(=O)c1ncc(-c2cccc(CC)c2)cc1NC(=O)CNCC1CCN(C)C(=O)C1] | (qx) |
| 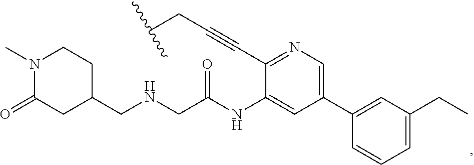 [SMILES: *CC#Cc1ncc(-c2cccc(CC)c2)cc1NC(=O)CNCC1CCN(C)C(=O)C1] | (qy) |
| 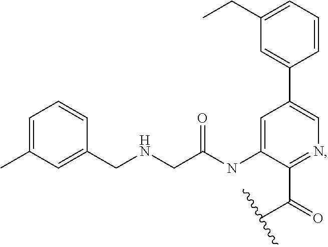 [SMILES: *C(=O)c1ncc(-c2cccc(CC)c2)cc1NC(=O)CNCc1cccc(C)c1] | (qz) |
| 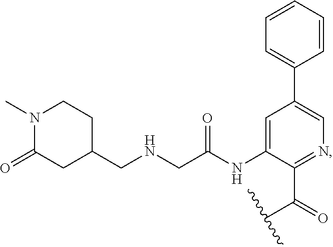 [SMILES: *C(=O)c1ncc(-c2ccccc2)cc1NC(=O)CNCC1CCN(C)C(=O)C1] | (ra) |
| 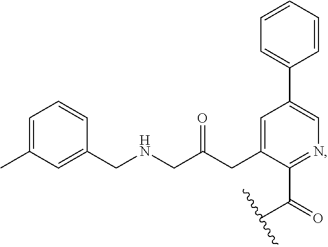 [SMILES: *C(=O)c1ncc(-c2ccccc2)cc1CC(=O)CNCc1cccc(C)c1] | (rb) |
| 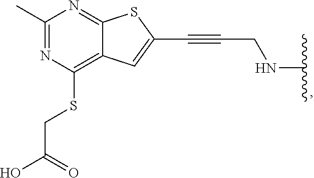 [SMILES: *NCC#Cc1cc2c(SCC(=O)O)nc(C)nc2s1] | (rc) |
| 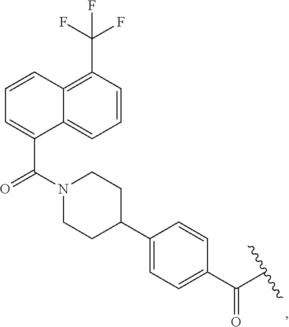 [SMILES: *C(=O)c1ccc(C2CCN(C(=O)c3cccc4c(C(F)(F)F)cccc34)CC2)cc1] | (rd) |
| 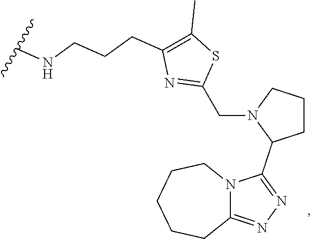 [SMILES: *NCCCc1nc(CN2CCCC2c2nnc3n2CCCCC3)sc1C] | (re) |
| 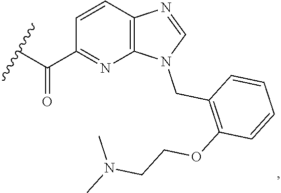 [SMILES: *C(=O)c1ccc2ncn(Cc3ccccc3OCCN(C)C)c2n1] | (rf) |
| 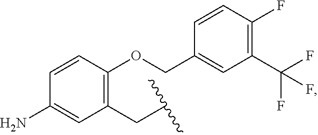 [SMILES: *Cc1cc(N)ccc1OCc1ccc(F)c(C(F)(F)F)c1] | (rg) |
| 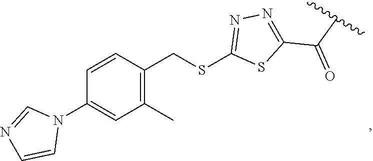 [SMILES: *C(=O)c1nnc(SCc2ccc(-n3ccnc3)cc2C)s1] | (rh) |
| 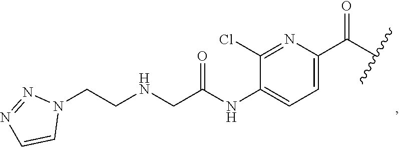 [SMILES: *C(=O)c1ccc(NC(=O)CNCCn2ccnn2)c(Cl)n1] | (ri) |
| 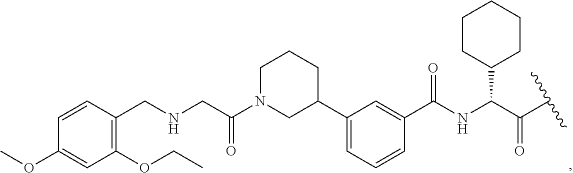 [SMILES: *C(=O)[C@H](NC(=O)c1cccc(C2CCCN(C(=O)CNCc3ccc(OC)cc3OCC)C2)c1)C1CCCCC1] | (rj) |
| 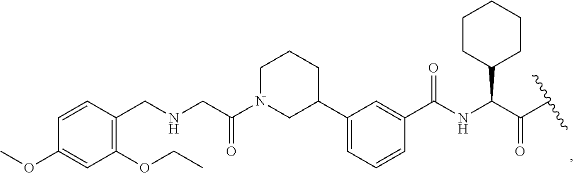 [SMILES: *C(=O)[C@@H](NC(=O)c1cccc(C2CCCN(C(=O)CNCc3ccc(OC)cc3OCC)C2)c1)C1CCCCC1] | (rk) |
| 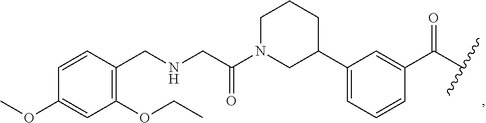 [SMILES: *C(=O)c1cccc(C2CCCN(C(=O)CNCc3ccc(OC)cc3OCC)C2)c1] | (rl) |
| 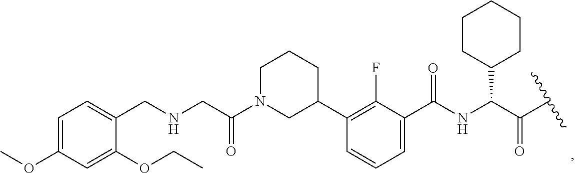 [SMILES: *C(=O)[C@H](NC(=O)c1cccc(C2CCCN(C(=O)CNCc3ccc(OC)cc3OCC)C2)c1F)C1CCCCC1] | (rm) |
| 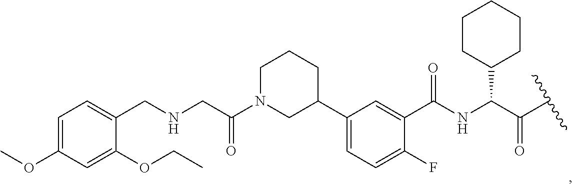 [SMILES: *C(=O)[C@H](NC(=O)c1cc(C2CCCN(C(=O)CNCc3ccc(OC)cc3OCC)C2)ccc1F)C1CCCCC1] | (rn) |
| 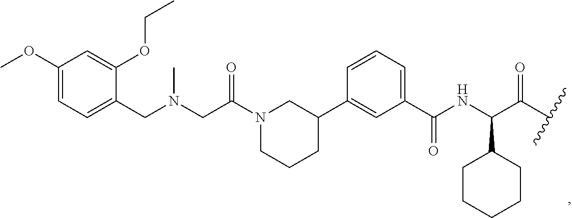 [SMILES: *C(=O)[C@H](NC(=O)c1cccc(C2CCCN(C(=O)CN(C)Cc3ccc(OC)cc3OCC)C2)c1)C1CCCCC1] | (ro) |
| 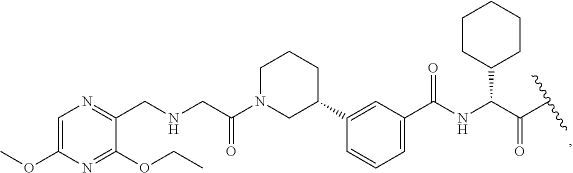 [SMILES: *C(=O)[C@H](NC(=O)c1cccc([C@H]2CCCN(C(=O)CNCc3ncc(OC)nc3OCC)C2)c1)C1CCCCC1] | (rp) |
| 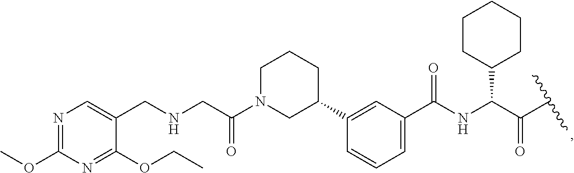 [SMILES: *C(=O)[C@H](NC(=O)c1cccc([C@H]2CCCN(C(=O)CNCc3cnc(OC)nc3OCC)C2)c1)C1CCCCC1] | (rq) |
| 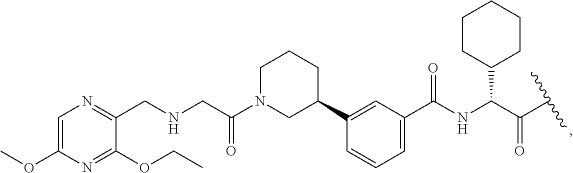 [SMILES: *C(=O)[C@H](NC(=O)c1cccc([C@@H]2CCCN(C(=O)CNCc3ncc(OC)nc3OCC)C2)c1)C1CCCCC1] | (rr) |
| 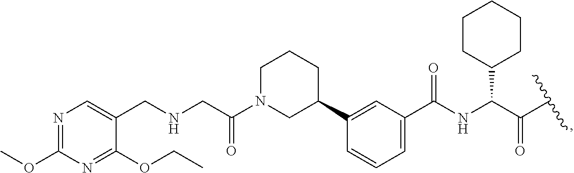 [SMILES: *C(=O)[C@H](NC(=O)c1cccc([C@@H]2CCCN(C(=O)CNCc3cnc(OC)nc3OCC)C2)c1)C1CCCCC1] | (rs) |
| 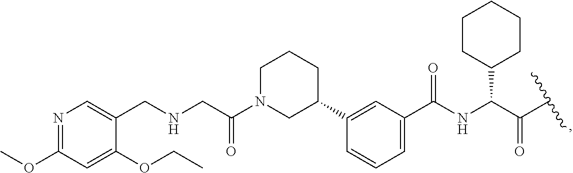 [SMILES: *C(=O)[C@H](NC(=O)c1cccc([C@H]2CCCN(C(=O)CNCc3cnc(OC)cc3OCC)C2)c1)C1CCCCC1] | (rt) |
| 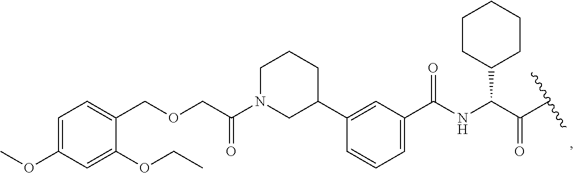 [SMILES: *C(=O)[C@H](NC(=O)c1cccc(C2CCCN(C(=O)COCc3ccc(OC)cc3OCC)C2)c1)C1CCCCC1] | (ru) |
| 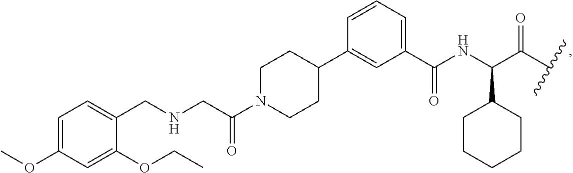 [SMILES: *C(=O)[C@H](NC(=O)c1cccc(C2CCN(C(=O)CNCc3ccc(OC)cc3OCC)CC2)c1)C1CCCCC1] | (rv) |
| 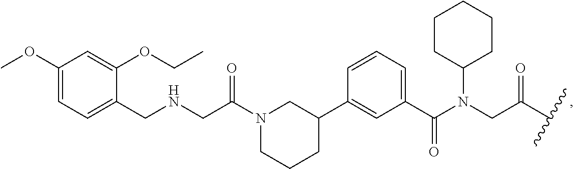 [SMILES: *C(=O)CN(C(=O)c1cccc(C2CCCN(C(=O)CNCc3ccc(OC)cc3OCC)C2)c1)C1CCCCC1 ; ","] | (rw) |
| 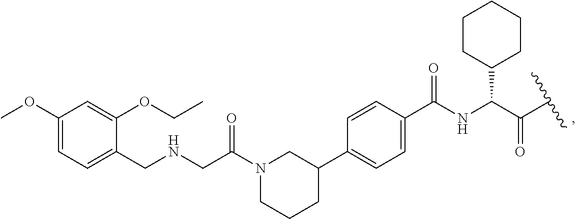 [SMILES: *C(=O)[C@H](NC(=O)c1ccc(C2CCCN(C(=O)CNCc3ccc(OC)cc3OCC)C2)cc1)C1CCCCC1 ; ","] | (rx) |
| 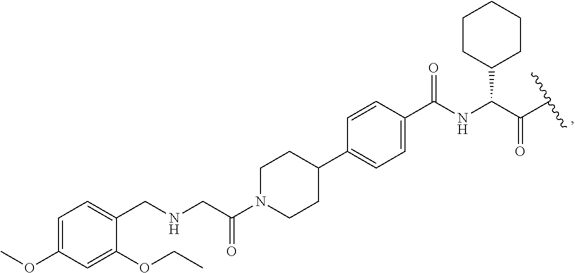 [SMILES: *C(=O)[C@H](NC(=O)c1ccc(C2CCN(C(=O)CNCc3ccc(OC)cc3OCC)CC2)cc1)C1CCCCC1 ; ","] | (ry) |
| 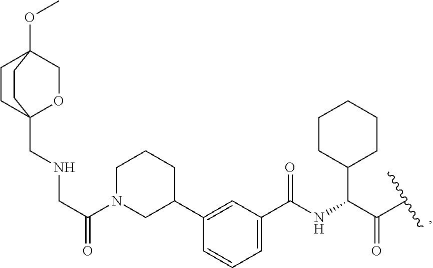 [SMILES: *C(=O)[C@H](NC(=O)c1cccc(C2CCCN(C(=O)CNCC34CCC(OC)(CC3)CO4)C2)c1)C1CCCCC1 ; ","] | (rz) |
| 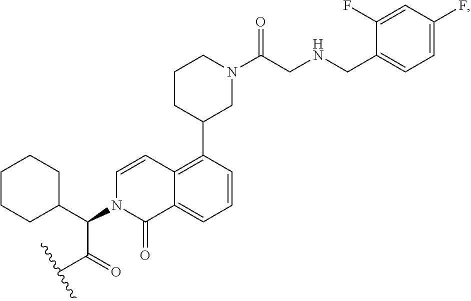 [SMILES: *C(=O)[C@@H](C1CCCCC1)n1ccc2c(C3CCCN(C(=O)CNCc4ccc(F)cc4F)C3)cccc2c1=O] | (sa) |
| 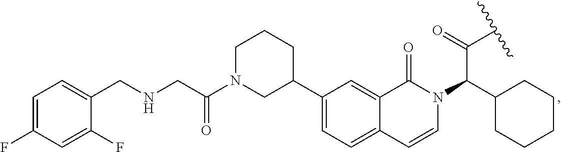 [SMILES: *C(=O)[C@@H](C1CCCCC1)n1ccc2ccc(C3CCCN(C(=O)CNCc4ccc(F)cc4F)C3)cc2c1=O ; ","] | (sb) |
| 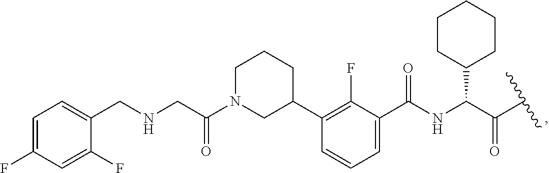 [SMILES: *C(=O)[C@H](NC(=O)c1cccc(C2CCCN(C(=O)CNCc3ccc(F)cc3F)C2)c1F)C1CCCCC1 ; ","] | (sc) |
| 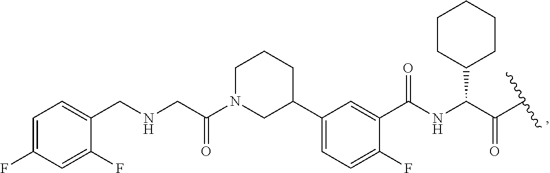 [SMILES: *C(=O)[C@H](NC(=O)c1cc(C2CCCN(C(=O)CNCc3ccc(F)cc3F)C2)ccc1F)C1CCCCC1 ; ","] | (sd) |
| 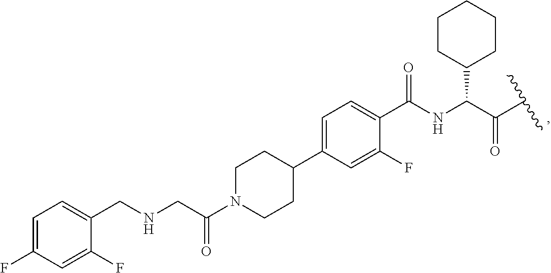 [SMILES: *C(=O)[C@H](NC(=O)c1ccc(C2CCN(C(=O)CNCc3ccc(F)cc3F)CC2)cc1F)C1CCCCC1 ; ","] | (se) |
| 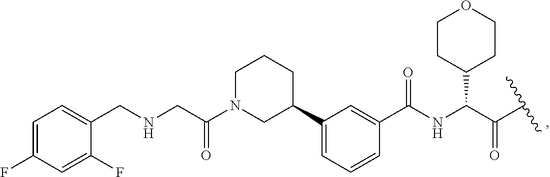 [SMILES: *C(=O)[C@H](NC(=O)c1cccc([C@@H]2CCCN(C(=O)CNCc3ccc(F)cc3F)C2)c1)C1CCOCC1 ; ","] | (sf) |
| 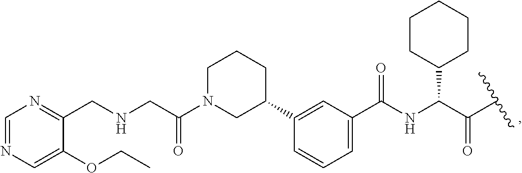 [SMILES: *C(=O)[C@H](NC(=O)c1cccc([C@H]2CCCN(C(=O)CNCc3ncncc3OCC)C2)c1)C1CCCCC1 ; ","] | (sg) |
| 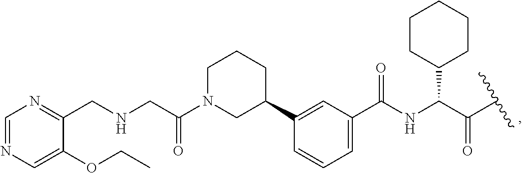 [SMILES: *C(=O)[C@H](NC(=O)c1cccc([C@@H]2CCCN(C(=O)CNCc3ncncc3OCC)C2)c1)C1CCCCC1 ; ","] | (sh) |
| 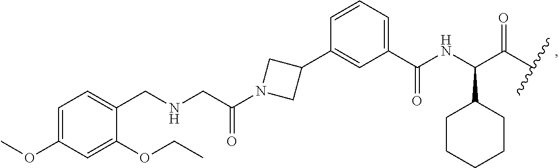 [SMILES: *C(=O)[C@H](NC(=O)c1cccc(C2CN(C(=O)CNCc3ccc(OC)cc3OCC)C2)c1)C1CCCCC1 ; ","] | (si) |
| 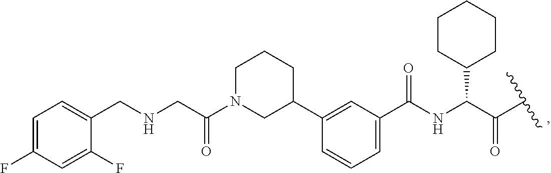 [SMILES: *C(=O)[C@H](NC(=O)c1cccc(C2CCCN(C(=O)CNCc3ccc(F)cc3F)C2)c1)C1CCCCC1 ; ","] | (sj) |
| 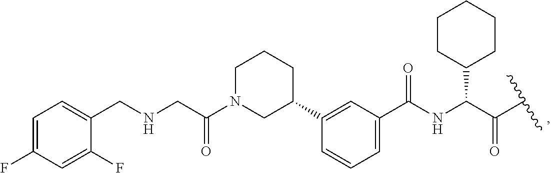 [SMILES: *C(=O)[C@H](NC(=O)c1cccc([C@H]2CCCN(C(=O)CNCc3ccc(F)cc3F)C2)c1)C1CCCCC1 ; ","] | (sk) |
| 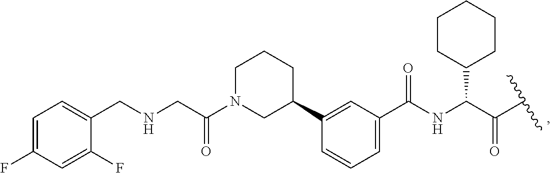 [SMILES: *C(=O)[C@H](NC(=O)c1cccc([C@@H]2CCCN(C(=O)CNCc3ccc(F)cc3F)C2)c1)C1CCCCC1 ; ","] | (sl) |
| 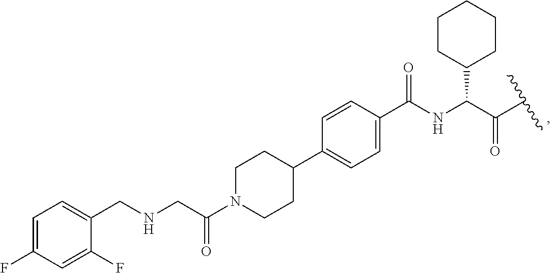 [SMILES: *C(=O)[C@H](NC(=O)c1ccc(C2CCN(C(=O)CNCc3ccc(F)cc3F)CC2)cc1)C1CCCCC1 ; ","] | (sm) |
| 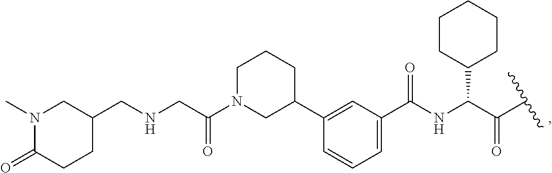 [SMILES: *C(=O)[C@H](NC(=O)c1cccc(C2CCCN(C(=O)CNCC3CCC(=O)N(C)C3)C2)c1)C1CCCCC1 ; ","] | (sn) |
| 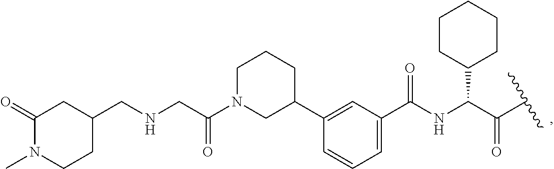 [SMILES: *C(=O)[C@H](NC(=O)c1cccc(C2CCCN(C(=O)CNCC3CCN(C)C(=O)C3)C2)c1)C1CCCCC1 ; ","] | (so) |
| 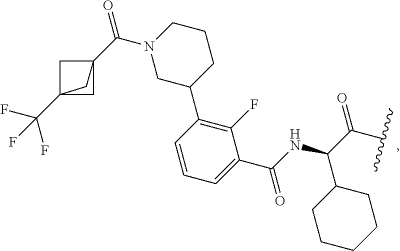 [SMILES: *C(=O)[C@H](NC(=O)c1cccc(C2CCCN(C(=O)C34CC(C(F)(F)F)(C3)C4)C2)c1F)C1CCCCC1 ; ","] | (sp) |
| 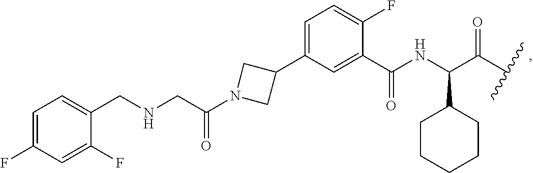 [SMILES: *C(=O)[C@H](NC(=O)c1cc(C2CN(C(=O)CNCc3ccc(F)cc3F)C2)ccc1F)C1CCCCC1 ; ","] | (sq) |
| 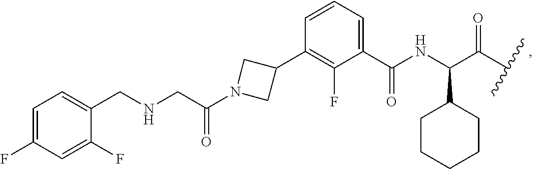 [SMILES: *C(=O)[C@H](NC(=O)c1cccc(C2CN(C(=O)CNCc3ccc(F)cc3F)C2)c1F)C1CCCCC1 ; ","] | (sr) |
| 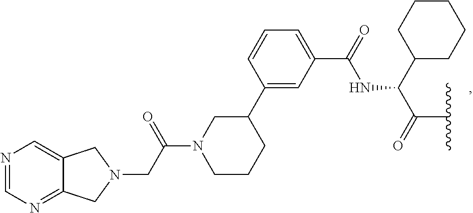 [SMILES: *C(=O)[C@H](NC(=O)c1cccc(C2CCCN(C(=O)CN3Cc4cncnc4C3)C2)c1)C1CCCCC1 ; ","] | (ss) |
| 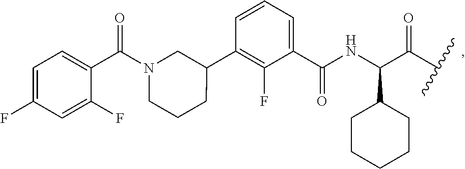 [SMILES: *C(=O)[C@H](NC(=O)c1cccc(C2CCCN(C(=O)c3ccc(F)cc3F)C2)c1F)C1CCCCC1 ; ","] | (st) |
| 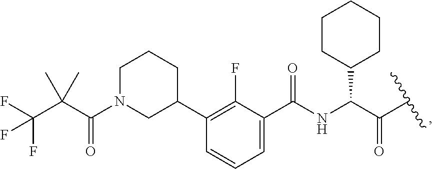 [SMILES: *C(=O)[C@H](NC(=O)c1cccc(C2CCCN(C(=O)C(C)(C)C(F)(F)F)C2)c1F)C1CCCCC1 ; ","] | (su) |
| 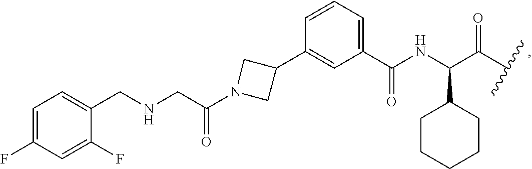 [SMILES: *C(=O)[C@H](NC(=O)c1cccc(C2CN(C(=O)CNCc3ccc(F)cc3F)C2)c1)C1CCCCC1 ; ","] | (sv) |
| 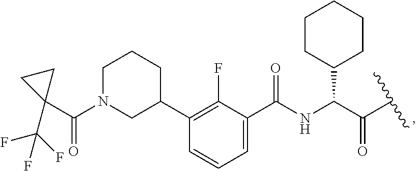 [SMILES: *C(=O)[C@H](NC(=O)c1cccc(C2CCCN(C(=O)C3(C(F)(F)F)CC3)C2)c1F)C1CCCCC1 ; ","] | (sw) |
| 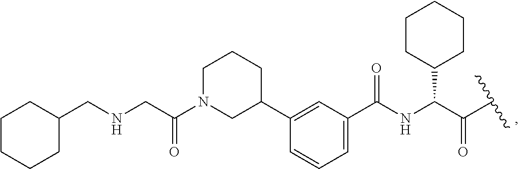 [SMILES: *C(=O)[C@H](NC(=O)c1cccc(C2CCCN(C(=O)CNCC3CCCCC3)C2)c1)C1CCCCC1 ; ","] | (sx) |
| 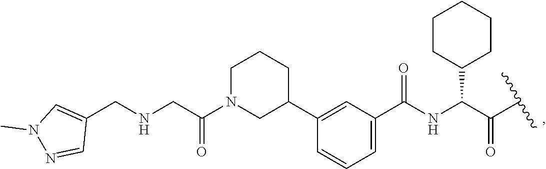 [SMILES: *C(=O)[C@H](NC(=O)c1cccc(C2CCCN(C(=O)CNCc3cnn(C)c3)C2)c1)C1CCCCC1 ; ","] | (sy) |
| 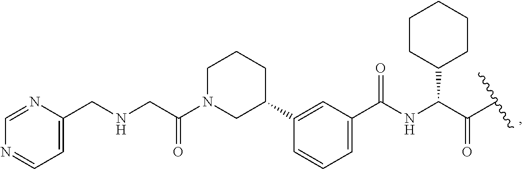 [SMILES: *C(=O)[C@H](NC(=O)c1cccc([C@H]2CCCN(C(=O)CNCc3ccncn3)C2)c1)C1CCCCC1 ; ","] | (sz) |
| 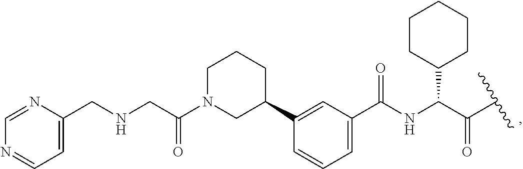 [SMILES: *C(=O)[C@H](NC(=O)c1cccc([C@@H]2CCCN(C(=O)CNCc3ccncn3)C2)c1)C1CCCCC1 ; ","] | (ta) |
| 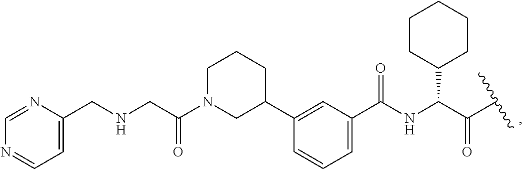 [SMILES: *C(=O)[C@H](NC(=O)c1cccc(C2CCCN(C(=O)CNCc3ccncn3)C2)c1)C1CCCCC1 ; ","] | (tb) |
| 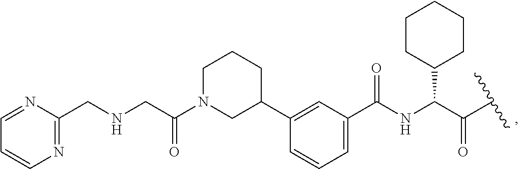 [SMILES: *C(=O)[C@H](NC(=O)c1cccc(C2CCCN(C(=O)CNCc3ncccn3)C2)c1)C1CCCCC1 ; ","] | (tc) |
| 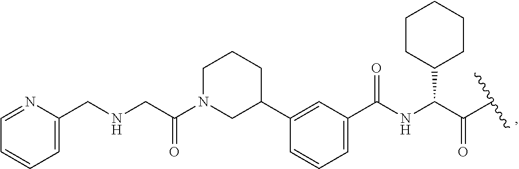 [SMILES: *C(=O)[C@H](NC(=O)c1cccc(C2CCCN(C(=O)CNCc3ccccn3)C2)c1)C1CCCCC1 ; ","] | (td) |
| 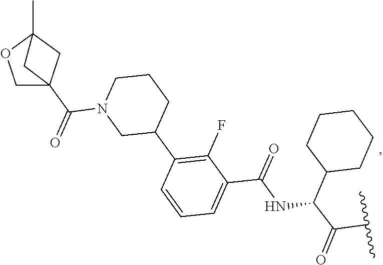 [SMILES: *C(=O)[C@H](NC(=O)c1cccc(C2CCCN(C(=O)C34COC(C)(C3)C4)C2)c1F)C1CCCCC1 ; ","] | (te) |
| 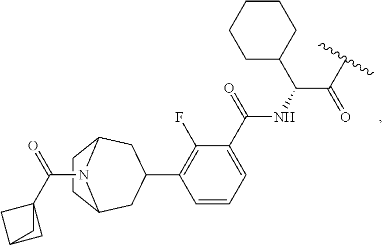 [SMILES: *C(=O)[C@H](NC(=O)c1cccc(C2CC3CCC(C2)N3C(=O)C23CC(C2)C3)c1F)C1CCCCC1 ; ","] | (tf) |
| 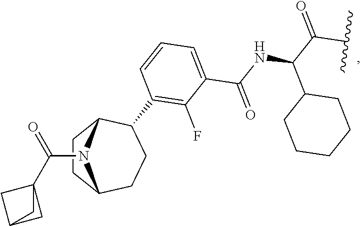 [SMILES: *C(=O)[C@H](NC(=O)c1cccc([C@@H]2CC[C@@H]3CC[C@H]2N3C(=O)C23CC(C2)C3)c1F)C1CCCCC1 ; ","] | (tg) |
| 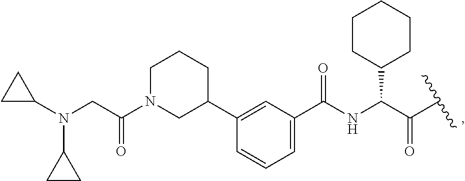 [SMILES: *C(=O)[C@H](NC(=O)c1cccc(C2CCCN(C(=O)CN(C3CC3)C3CC3)C2)c1)C1CCCCC1 ; ","] | (th) |
| 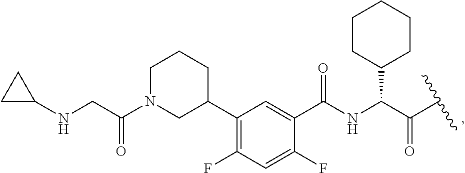 [SMILES: *C(=O)[C@H](NC(=O)c1cc(C2CCCN(C(=O)CNC3CC3)C2)c(F)cc1F)C1CCCCC1 ; ","] | (ti) |
| 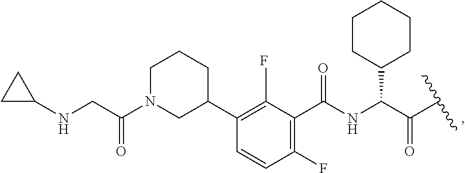 | (tj) |
| 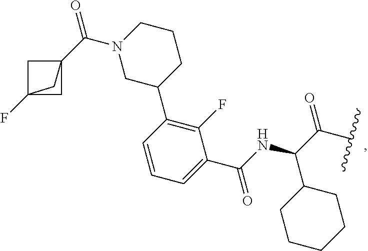 [SMILES: *C(=O)[C@H](NC(=O)c1cccc(C2CCCN(C(=O)C34CC(F)(C3)C4)C2)c1F)C1CCCCC1 ; ","] | (tk) |
| 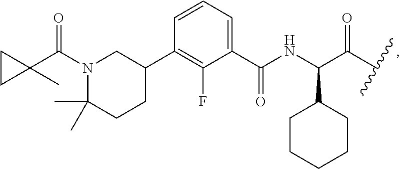 [SMILES: *C(=O)[C@H](NC(=O)c1cccc(C2CCC(C)(C)N(C(=O)C3(C)CC3)C2)c1F)C1CCCCC1 ; ","] | (tl) |
| 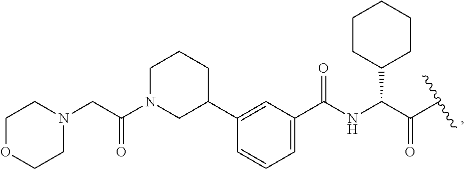 [SMILES: *C(=O)[C@H](NC(=O)c1cccc(C2CCCN(C(=O)CN3CCOCC3)C2)c1)C1CCCCC1 ; ","] | (tm) |
| 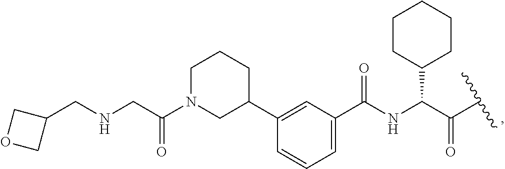 [SMILES: *C(=O)[C@H](NC(=O)c1cccc(C2CCCN(C(=O)CNCC3COC3)C2)c1)C1CCCCC1 ; ","] | (tn) |
| 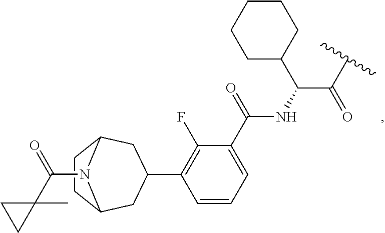 [SMILES: *C(=O)[C@H](NC(=O)c1cccc(C2CC3CCC(C2)N3C(=O)C2(C)CC2)c1F)C1CCCCC1 ; ","] | (to) |
| 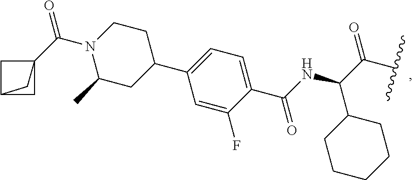 [SMILES: *C(=O)[C@H](NC(=O)c1ccc(C2CCN(C(=O)C34CC(C3)C4)[C@H](C)C2)cc1F)C1CCCCC1 ; ","] | (tp) |
| 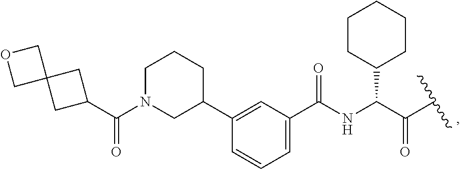 [SMILES: *C(=O)[C@H](NC(=O)c1cccc(C2CCCN(C(=O)C3CC4(COC4)C3)C2)c1)C1CCCCC1 ; ","] | (tq) |
| 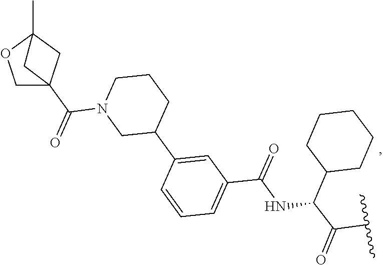 [SMILES: *C(=O)[C@H](NC(=O)c1cccc(C2CCCN(C(=O)C34COC(C)(C3)C4)C2)c1)C1CCCCC1 ; ","] | (tr) |
| 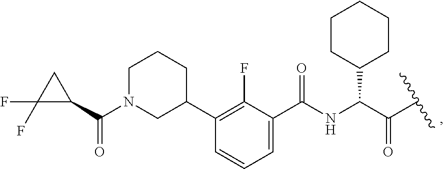 [SMILES: *C(=O)[C@H](NC(=O)c1cccc(C2CCCN(C(=O)[C@@H]3CC3(F)F)C2)c1F)C1CCCCC1 ; ","] | (ts) |
| 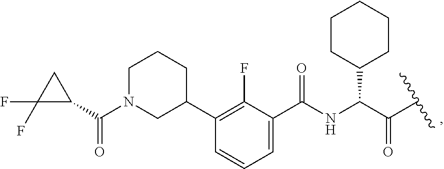 [SMILES: *C(=O)[C@H](NC(=O)c1cccc(C2CCCN(C(=O)[C@H]3CC3(F)F)C2)c1F)C1CCCCC1 ; ","] | (tt) |
| 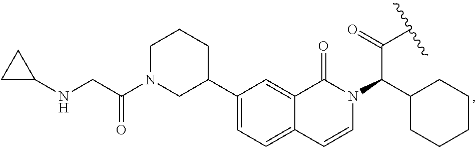 [SMILES: *C(=O)[C@@H](C1CCCCC1)n1ccc2ccc(C3CCCN(C(=O)CNC4CC4)C3)cc2c1=O ; ","] | (tu) |
| 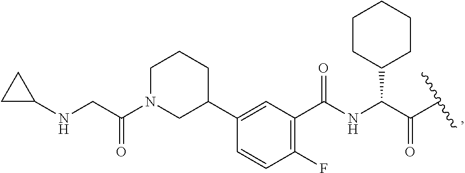 [SMILES: *C(=O)[C@H](NC(=O)c1cc(C2CCCN(C(=O)CNC3CC3)C2)ccc1F)C1CCCCC1 ; ","] | (tv) |
| 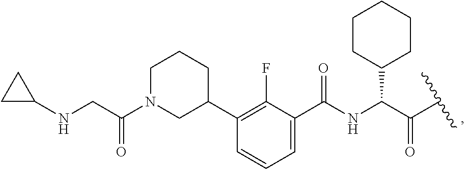 [SMILES: *C(=O)[C@H](NC(=O)c1cccc(C2CCCN(C(=O)CNC3CC3)C2)c1F)C1CCCCC1 ; ","] | (tw) |
| 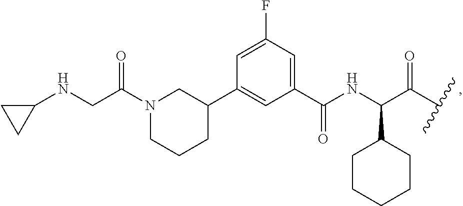 [SMILES: *C(=O)[C@H](NC(=O)c1cc(F)cc(C2CCCN(C(=O)CNC3CC3)C2)c1)C1CCCCC1 ; ","] | (tx) |
| 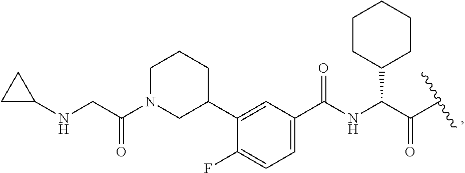 [SMILES: *C(=O)[C@H](NC(=O)c1ccc(F)c(C2CCCN(C(=O)CNC3CC3)C2)c1)C1CCCCC1 ; ","] | (ty) |
| 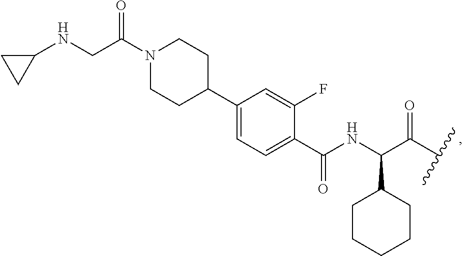 [SMILES: *C(=O)[C@H](NC(=O)c1ccc(C2CCN(C(=O)CNC3CC3)CC2)cc1F)C1CCCCC1 ; ","] | (tz) |
| 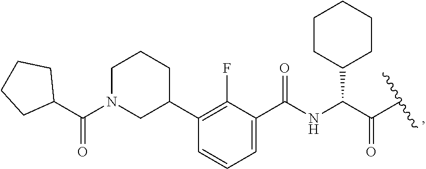 [SMILES: *C(=O)[C@H](NC(=O)c1cccc(C2CCCN(C(=O)C3CCCC3)C2)c1F)C1CCCCC1 ; ","] | (ua) |
| 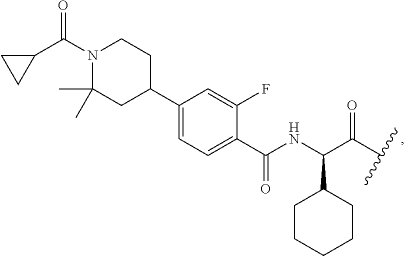 [SMILES: *C(=O)[C@H](NC(=O)c1ccc(C2CCN(C(=O)C3CC3)C(C)(C)C2)cc1F)C1CCCCC1 ; ","] | (ub) |
| 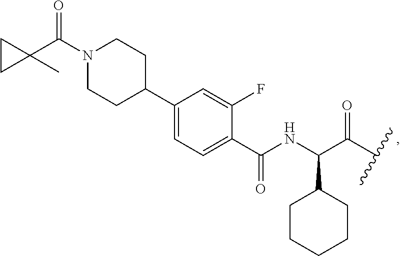 [SMILES: *C(=O)[C@H](NC(=O)c1ccc(C2CCN(C(=O)C3(C)CC3)CC2)cc1F)C1CCCCC1 ; ","] | (uc) |
| 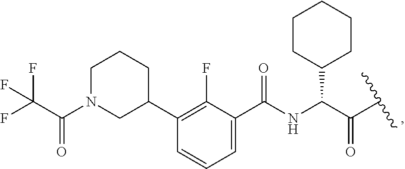 [SMILES: *C(=O)[C@H](NC(=O)c1cccc(C2CCCN(C(=O)C(F)(F)F)C2)c1F)C1CCCCC1 ; ","] | (ud) |
| 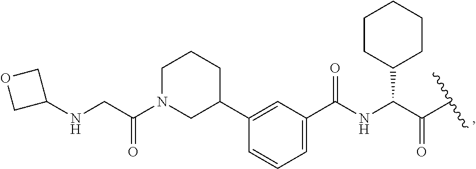 [SMILES: *C(=O)[C@H](NC(=O)c1cccc(C2CCCN(C(=O)CNC3COC3)C2)c1)C1CCCCC1 ; ","] | (ue) |
| 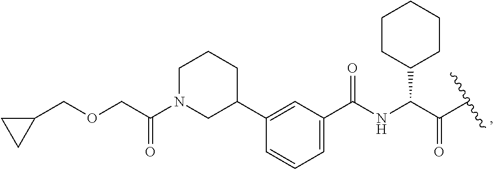 [SMILES: *C(=O)[C@H](NC(=O)c1cccc(C2CCCN(C(=O)COCC3CC3)C2)c1)C1CCCCC1 ; ","] | (uf) |
| 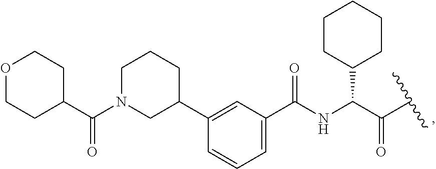 [SMILES: *C(=O)[C@H](NC(=O)c1cccc(C2CCCN(C(=O)C3CCOCC3)C2)c1)C1CCCCC1 ; ","] | (ug) |
| 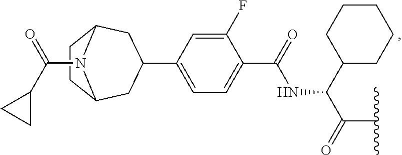 [SMILES: *C(=O)[C@H](NC(=O)c1ccc(C2CC3CCC(C2)N3C(=O)C2CC2)cc1F)C1CCCCC1 ; ","] | (uh) |
| 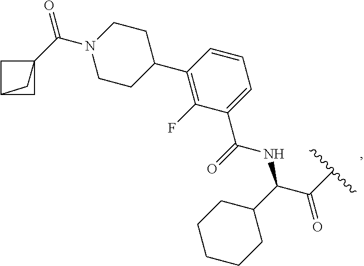 [SMILES: *C(=O)[C@H](NC(=O)c1cccc(C2CCN(C(=O)C34CC(C3)C4)CC2)c1F)C1CCCCC1 ; ","] | (ui) |
| 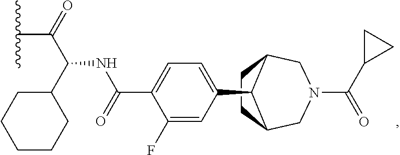 [SMILES: *C(=O)[C@H](NC(=O)c1ccc([C@@H]2[C@@H]3CC[C@H]2CN(C(=O)C2CC2)C3)cc1F)C1CCCCC1 ; ","] | (uj) |
| 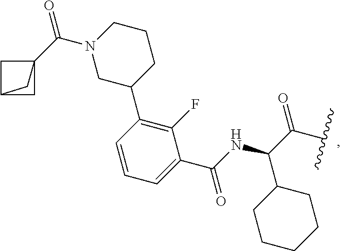 [SMILES: *C(=O)[C@H](NC(=O)c1cccc(C2CCCN(C(=O)C34CC(C3)C4)C2)c1F)C1CCCCC1 ; ","] | (uk) |
| 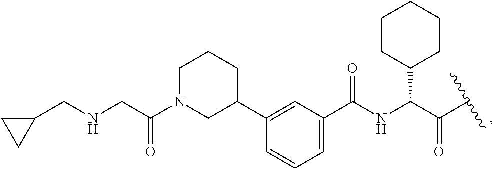 [SMILES: *C(=O)[C@H](NC(=O)c1cccc(C2CCCN(C(=O)CNCC3CC3)C2)c1)C1CCCCC1 ; ","] | (ul) |
| 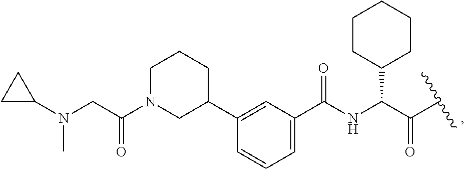 [SMILES: *C(=O)[C@H](NC(=O)c1cccc(C2CCCN(C(=O)CN(C)C3CC3)C2)c1)C1CCCCC1 ; ","] | (um) |
| 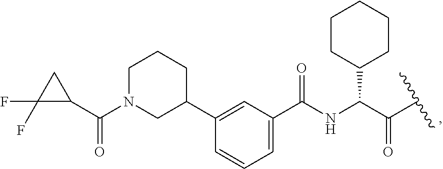 [SMILES: *C(=O)[C@H](NC(=O)c1cccc(C2CCCN(C(=O)C3CC3(F)F)C2)c1)C1CCCCC1 ; ","] | (un) |
| 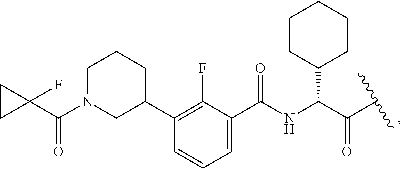 [SMILES: *C(=O)[C@H](NC(=O)c1cccc(C2CCCN(C(=O)C3(F)CC3)C2)c1F)C1CCCCC1 ; ","] | (uo) |
| 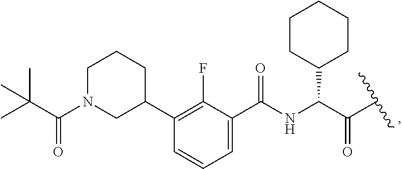 [SMILES: *C(=O)[C@H](NC(=O)c1cccc(C2CCCN(C(=O)C(C)(C)C)C2)c1F)C1CCCCC1 ; ","] | (up) |
| 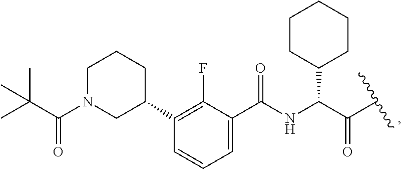 [SMILES: *C(=O)[C@H](NC(=O)c1cccc([C@H]2CCCN(C(=O)C(C)(C)C)C2)c1F)C1CCCCC1 ; ","] | (uq) |
| 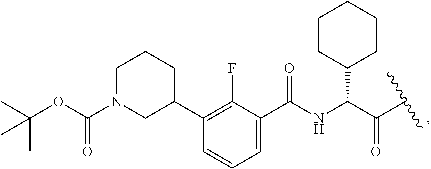 [SMILES: *C(=O)[C@H](NC(=O)c1cccc(C2CCCN(C(=O)OC(C)(C)C)C2)c1F)C1CCCCC1 ; ","] | (ur) |
| 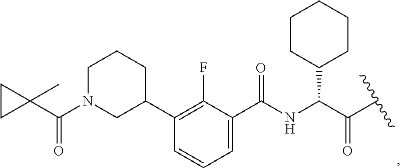 [SMILES: *C(=O)[C@H](NC(=O)c1cccc(C2CCCN(C(=O)C3(C)CC3)C2)c1F)C1CCCCC1] | (us) |
| 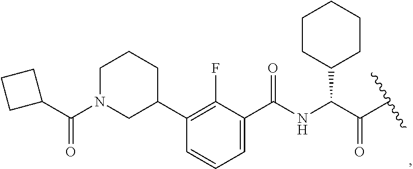 [SMILES: *C(=O)[C@H](NC(=O)c1cccc(C2CCCN(C(=O)C3CCC3)C2)c1F)C1CCCCC1] | (ut) |
| 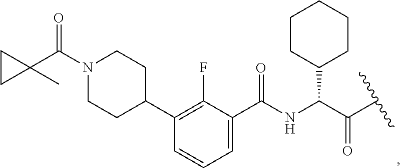 [SMILES: *C(=O)[C@H](NC(=O)c1cccc(C2CCN(C(=O)C3(C)CC3)CC2)c1F)C1CCCCC1] | (uu) |
| 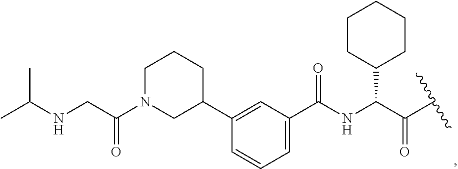 [SMILES: *C(=O)[C@H](NC(=O)c1cccc(C2CCCN(C(=O)CNC(C)C)C2)c1)C1CCCCC1] | (uv) |
| 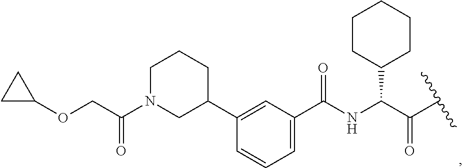 [SMILES: *C(=O)[C@H](NC(=O)c1cccc(C2CCCN(C(=O)COC3CC3)C2)c1)C1CCCCC1] | (uw) |
| 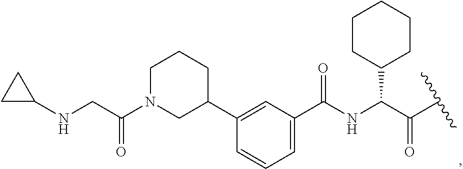 [SMILES: *C(=O)[C@H](NC(=O)c1cccc(C2CCCN(C(=O)CNC3CC3)C2)c1)C1CCCCC1] | (ux) |
| 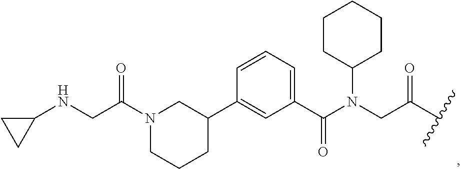 [SMILES: *C(=O)CN(C(=O)c1cccc(C2CCCN(C(=O)CNC3CC3)C2)c1)C1CCCCC1] | (uy) |
| 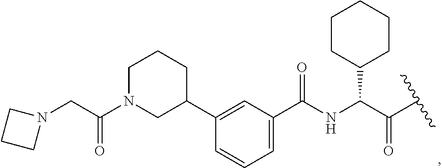 [SMILES: *C(=O)[C@H](NC(=O)c1cccc(C2CCCN(C(=O)CN3CCC3)C2)c1)C1CCCCC1] | (uz) |
| 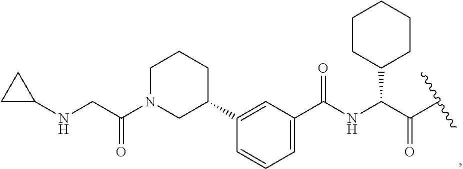 [SMILES: *C(=O)[C@H](NC(=O)c1cccc([C@H]2CCCN(C(=O)CNC3CC3)C2)c1)C1CCCCC1] | (va) |
| 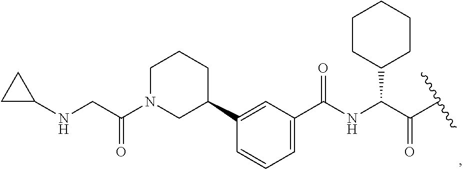 [SMILES: *C(=O)[C@H](NC(=O)c1cccc([C@@H]2CCCN(C(=O)CNC3CC3)C2)c1)C1CCCCC1] | (vb) |
| 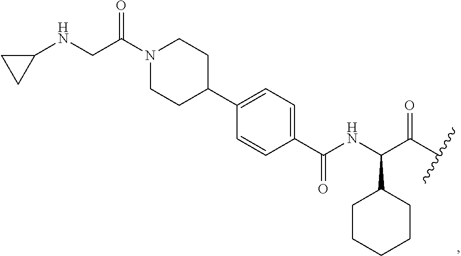 [SMILES: *C(=O)[C@H](NC(=O)c1ccc(C2CCN(C(=O)CNC3CC3)CC2)cc1)C1CCCCC1] | (vc) |
| 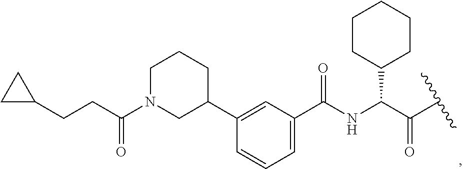 [SMILES: *C(=O)[C@H](NC(=O)c1cccc(C2CCCN(C(=O)CCC3CC3)C2)c1)C1CCCCC1] | (vd) |
| 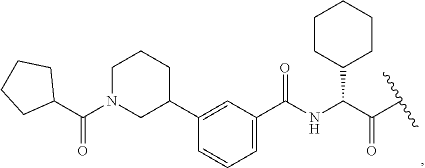 [SMILES: *C(=O)[C@H](NC(=O)c1cccc(C2CCCN(C(=O)C3CCCC3)C2)c1)C1CCCCC1] | (ve) |
| 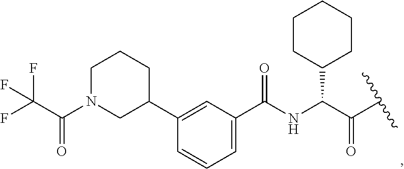 [SMILES: *C(=O)[C@H](NC(=O)c1cccc(C2CCCN(C(=O)C(F)(F)F)C2)c1)C1CCCCC1] | (vf) |
| 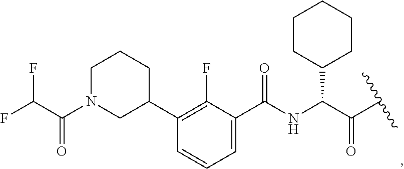 [SMILES: *C(=O)[C@H](NC(=O)c1cccc(C2CCCN(C(=O)C(F)F)C2)c1F)C1CCCCC1] | (vg) |
| 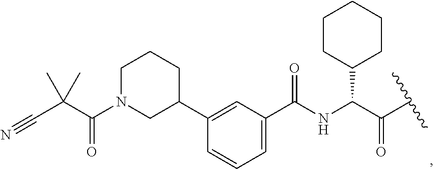 [SMILES: *C(=O)[C@H](NC(=O)c1cccc(C2CCCN(C(=O)C(C)(C)C#N)C2)c1)C1CCCCC1] | (vh) |
| 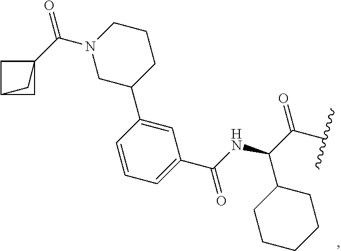 [SMILES: *C(=O)[C@H](NC(=O)c1cccc(C2CCCN(C(=O)C34CC(C3)C4)C2)c1)C1CCCCC1] | (vi) |
| 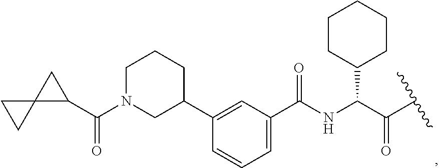 [SMILES: *C(=O)[C@H](NC(=O)c1cccc(C2CCCN(C(=O)C3CC34CC4)C2)c1)C1CCCCC1] | (vj) |
| 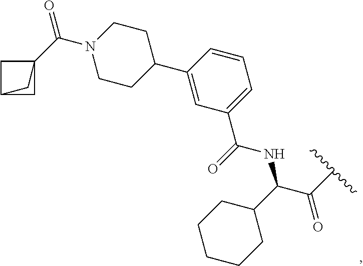 [SMILES: *C(=O)[C@H](NC(=O)c1cccc(C2CCN(C(=O)C34CC(C3)C4)CC2)c1)C1CCCCC1] | (vk) |
| 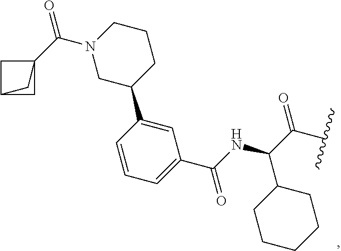 [SMILES: *C(=O)[C@H](NC(=O)c1cccc([C@@H]2CCCN(C(=O)C34CC(C3)C4)C2)c1)C1CCCCC1] | (vl) |
| 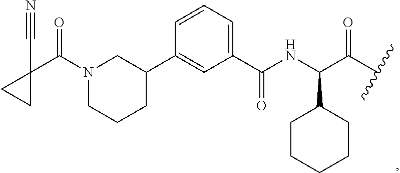 [SMILES: *C(=O)[C@H](NC(=O)c1cccc(C2CCCN(C(=O)C3(C#N)CC3)C2)c1)C1CCCCC1] | (vm) |
| 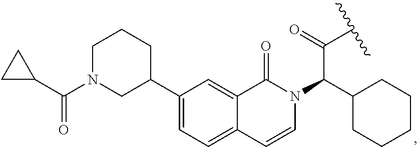 [SMILES: *C(=O)[C@@H](C1CCCCC1)n1ccc2ccc(C3CCCN(C(=O)C4CC4)C3)cc2c1=O] | (vn) |
| 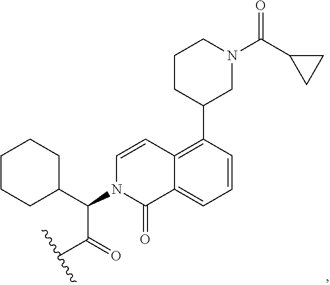 [SMILES: *C(=O)[C@@H](C1CCCCC1)n1ccc2c(C3CCCN(C(=O)C4CC4)C3)cccc2c1=O] | (vo) |
| 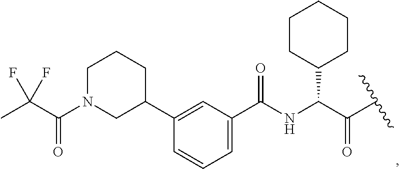 [SMILES: *C(=O)[C@H](NC(=O)c1cccc(C2CCCN(C(=O)C(C)(F)F)C2)c1)C1CCCCC1] | (vp) |
| 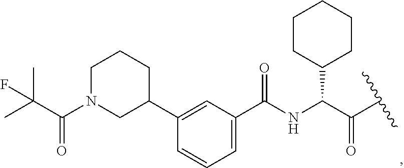 [SMILES: *C(=O)[C@H](NC(=O)c1cccc(C2CCCN(C(=O)C(C)(C)F)C2)c1)C1CCCCC1] | (vq) |
| 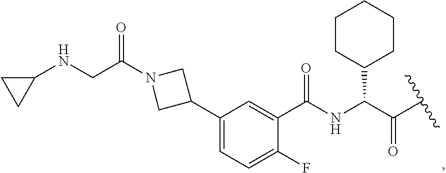 [SMILES: *C(=O)[C@H](NC(=O)c1cc(C2CN(C(=O)CNC3CC3)C2)ccc1F)C1CCCCC1] | (vr) |
| 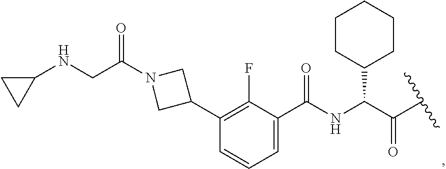 [SMILES: *C(=O)[C@H](NC(=O)c1cccc(C2CN(C(=O)CNC3CC3)C2)c1F)C1CCCCC1] | (vs) |
| 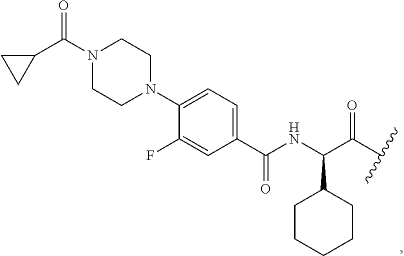 [SMILES: *C(=O)[C@H](NC(=O)c1ccc(N2CCN(C(=O)C3CC3)CC2)c(F)c1)C1CCCCC1] | (vt) |
| 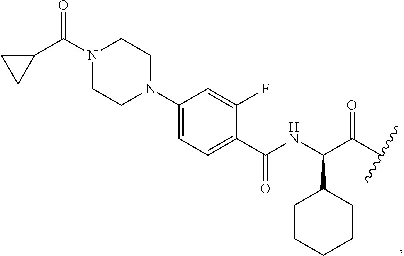 [SMILES: *C(=O)[C@H](NC(=O)c1ccc(N2CCN(C(=O)C3CC3)CC2)cc1F)C1CCCCC1] | (vu) |
| 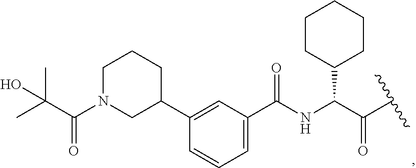 [SMILES: *C(=O)[C@H](NC(=O)c1cccc(C2CCCN(C(=O)C(C)(C)O)C2)c1)C1CCCCC1] | (vv) |
| 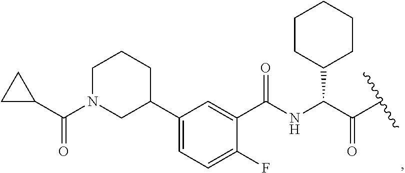 [SMILES: *C(=O)[C@H](NC(=O)c1cc(C2CCCN(C(=O)C3CC3)C2)ccc1F)C1CCCCC1] | (vw) |
| 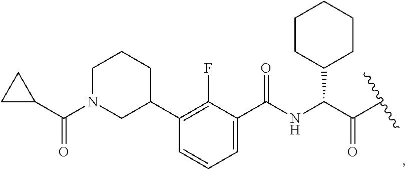 [SMILES: *C(=O)[C@H](NC(=O)c1cccc(C2CCCN(C(=O)C3CC3)C2)c1F)C1CCCCC1] | (vx) |
| 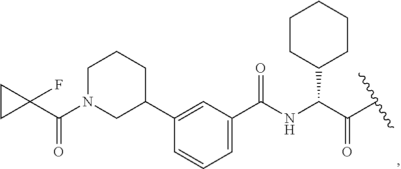 [SMILES: *C(=O)[C@H](NC(=O)c1cccc(C2CCCN(C(=O)C3(F)CC3)C2)c1)C1CCCCC1] | (vy) |
| 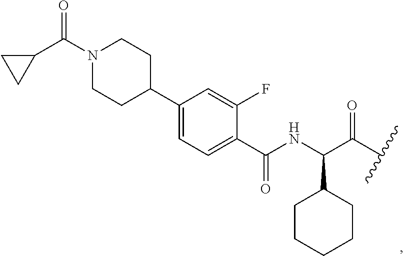 [SMILES: *C(=O)[C@H](NC(=O)c1ccc(C2CCN(C(=O)C3CC3)CC2)cc1F)C1CCCCC1] | (vz) |
| 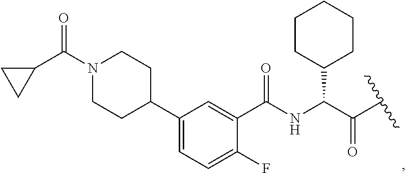 [SMILES: *C(=O)[C@H](NC(=O)c1cc(C2CCN(C(=O)C3CC3)CC2)ccc1F)C1CCCCC1] | (wa) |
| 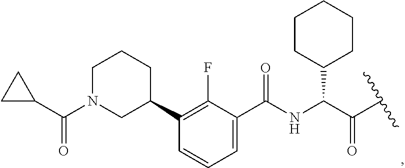 [SMILES: *C(=O)[C@H](NC(=O)c1cccc([C@@H]2CCCN(C(=O)C3CC3)C2)c1F)C1CCCCC1] | (wb) |
| 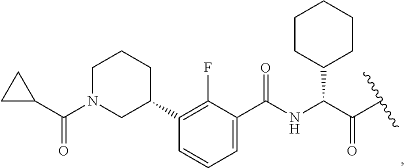 [SMILES: *C(=O)[C@H](NC(=O)c1cccc([C@H]2CCCN(C(=O)C3CC3)C2)c1F)C1CCCCC1] | (wc) |
| 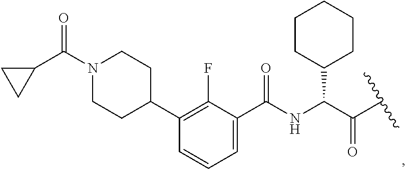 [SMILES: *C(=O)[C@H](NC(=O)c1cccc(C2CCN(C(=O)C3CC3)CC2)c1F)C1CCCCC1] | (wd) |
| 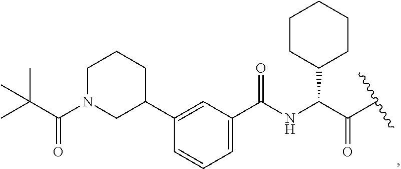 [SMILES: *C(=O)[C@H](NC(=O)c1cccc(C2CCCN(C(=O)C(C)(C)C)C2)c1)C1CCCCC1] | (we) |
| 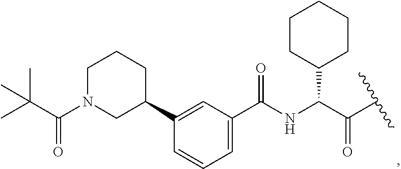 [SMILES: *C(=O)[C@H](NC(=O)c1cccc([C@@H]2CCCN(C(=O)C(C)(C)C)C2)c1)C1CCCCC1] | (wf) |
| 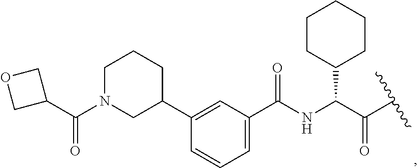 [SMILES: *C(=O)[C@H](NC(=O)c1cccc(C2CCCN(C(=O)C3COC3)C2)c1)C1CCCCC1] | (wg) |
| 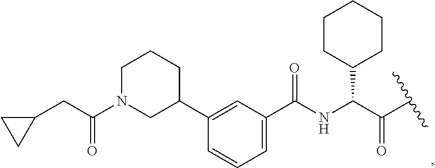 [SMILES: *C(=O)[C@H](NC(=O)c1cccc(C2CCCN(C(=O)CC3CC3)C2)c1)C1CCCCC1] | (wh) |
| 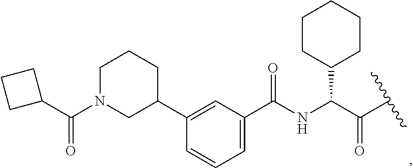 [SMILES: *C(=O)[C@H](NC(=O)c1cccc(C2CCCN(C(=O)C3CCC3)C2)c1)C1CCCCC1] | (wi) |
| 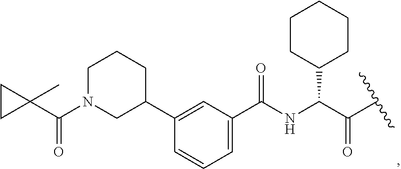 [SMILES: *C(=O)[C@H](NC(=O)c1cccc(C2CCCN(C(=O)C3(C)CC3)C2)c1)C1CCCCC1] | (wj) |
| 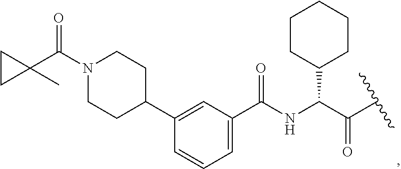 [SMILES: *C(=O)[C@H](NC(=O)c1cccc(C2CCN(C(=O)C3(C)CC3)CC2)c1)C1CCCCC1] | (wk) |
| 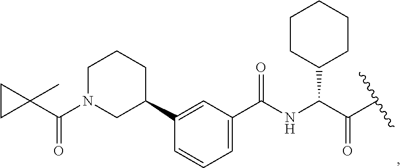 [SMILES: *C(=O)[C@H](NC(=O)c1cccc([C@@H]2CCCN(C(=O)C3(C)CC3)C2)c1)C1CCCCC1] | (wl) |
| 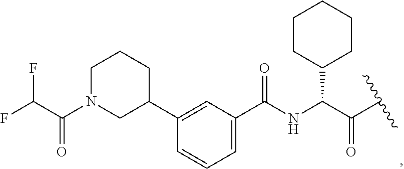 [SMILES: *C(=O)[C@H](NC(=O)c1cccc(C2CCCN(C(=O)C(F)F)C2)c1)C1CCCCC1] | (wm) |
| 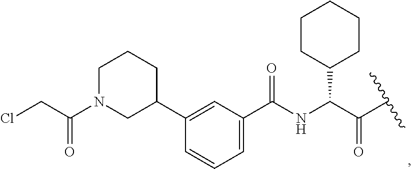 [SMILES: *C(=O)[C@H](NC(=O)c1cccc(C2CCCN(C(=O)CCl)C2)c1)C1CCCCC1] | (wn) |
| 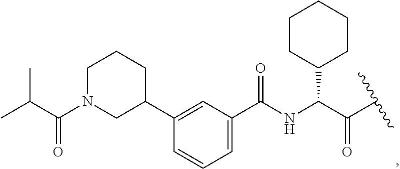 [SMILES: *C(=O)[C@H](NC(=O)c1cccc(C2CCCN(C(=O)C(C)C)C2)c1)C1CCCCC1] | (wo) |
| 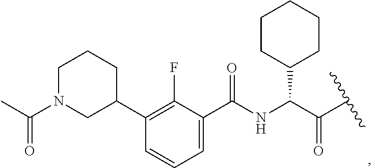 [SMILES: *C(=O)[C@H](NC(=O)c1cccc(C2CCCN(C(C)=O)C2)c1F)C1CCCCC1] | (wp) |
| 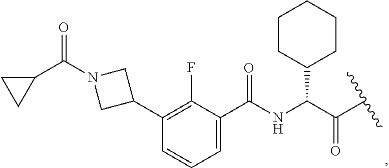 [SMILES: *C(=O)[C@H](NC(=O)c1cccc(C2CN(C(=O)C3CC3)C2)c1F)C1CCCCC1] | (wq) |
| 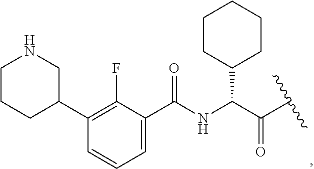 [SMILES: *C(=O)[C@H](NC(=O)c1cccc(C2CCCNC2)c1F)C1CCCCC1] | (wr) |
| 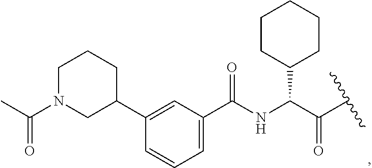 [SMILES: *C(=O)[C@H](NC(=O)c1cccc(C2CCCN(C(C)=O)C2)c1)C1CCCCC1] | (ws) |
| 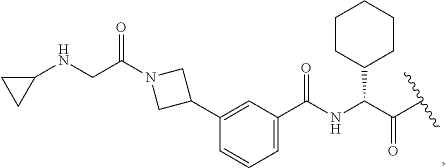 [SMILES: *C(=O)[C@H](NC(=O)c1cccc(C2CN(C(=O)CNC3CC3)C2)c1)C1CCCCC1] | (wt) |
| 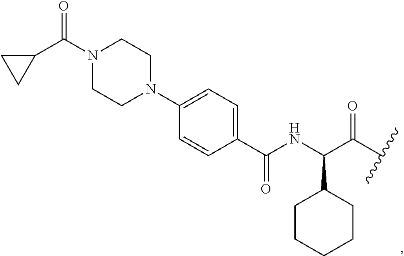 [SMILES: *C(=O)[C@H](NC(=O)c1ccc(N2CCN(C(=O)C3CC3)CC2)cc1)C1CCCCC1] | (wu) |
| 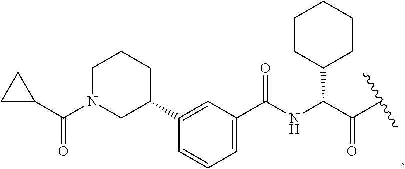 [SMILES: *C(=O)[C@H](NC(=O)c1cccc([C@H]2CCCN(C(=O)C3CC3)C2)c1)C1CCCCC1] | (wv) |
| 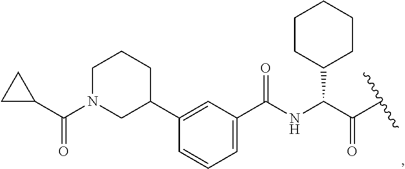 [SMILES: *C(=O)[C@H](NC(=O)c1cccc(C2CCCN(C(=O)C3CC3)C2)c1)C1CCCCC1] | (ww) |
| 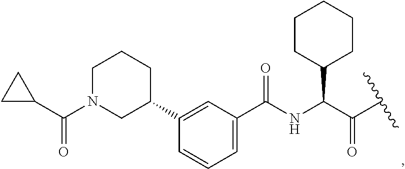 [SMILES: *C(=O)[C@@H](NC(=O)c1cccc([C@H]2CCCN(C(=O)C3CC3)C2)c1)C1CCCCC1] | (wx) |
| 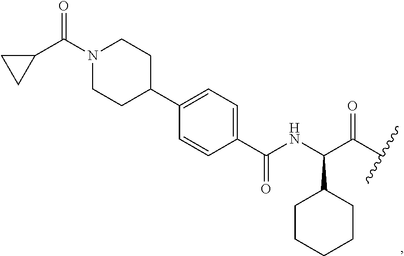 [SMILES: *C(=O)[C@H](NC(=O)c1ccc(C2CCN(C(=O)C3CC3)CC2)cc1)C1CCCCC1] | (wy) |
| 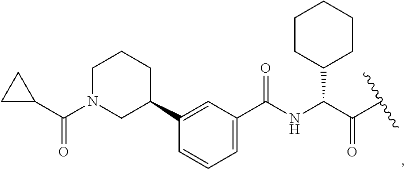 [SMILES: *C(=O)[C@H](NC(=O)c1cccc([C@@H]2CCCN(C(=O)C3CC3)C2)c1)C1CCCCC1] | (wz) |
| 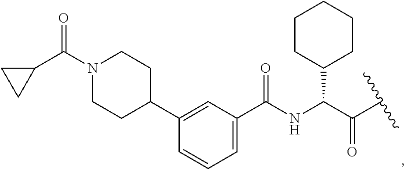 [SMILES: *C(=O)[C@H](NC(=O)c1cccc(C2CCN(C(=O)C3CC3)CC2)c1)C1CCCCC1] | (xa) |
| 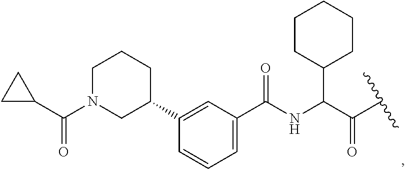 [SMILES: *C(=O)C(NC(=O)c1cccc([C@H]2CCCN(C(=O)C3CC3)C2)c1)C1CCCCC1] | (xb) |
| 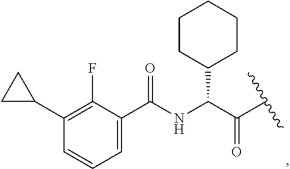 [SMILES: *C(=O)[C@H](NC(=O)c1cccc(C2CC2)c1F)C1CCCCC1] | (xc) |
| 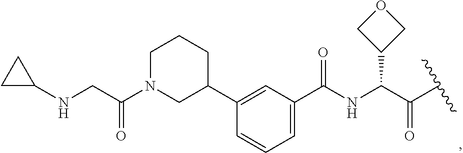 [SMILES: *C(=O)[C@H](NC(=O)c1cccc(C2CCCN(C(=O)CNC3CC3)C2)c1)C1COC1] | (xd) |
| 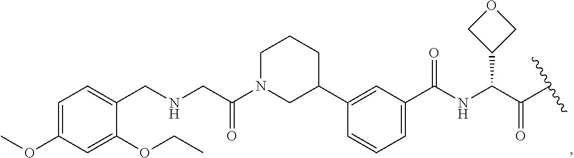 [SMILES: *C(=O)[C@H](NC(=O)c1cccc(C2CCCN(C(=O)CNCc3ccc(OC)cc3OCC)C2)c1)C1COC1] | (xe) |
| 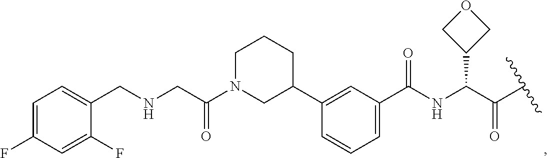 [SMILES: *C(=O)[C@H](NC(=O)c1cccc(C2CCCN(C(=O)CNCc3ccc(F)cc3F)C2)c1)C1COC1] | (xf) |
| 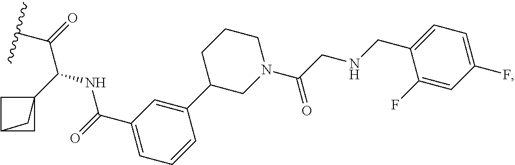 [SMILES: *C(=O)[C@H](NC(=O)c1cccc(C2CCCN(C(=O)CNCc3ccc(F)cc3F)C2)c1)C12CC(C1)C2] | (xg) |
| 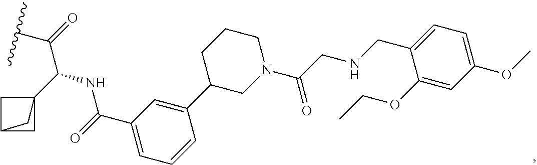 [SMILES: *C(=O)[C@H](NC(=O)c1cccc(C2CCCN(C(=O)CNCc3ccc(OC)cc3OCC)C2)c1)C12CC(C1)C2] | (xh) |
| 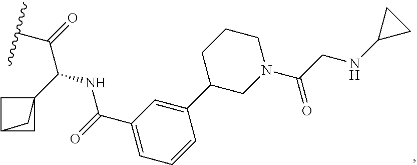 [SMILES: *C(=O)[C@H](NC(=O)c1cccc(C2CCCN(C(=O)CNC3CC3)C2)c1)C12CC(C1)C2] | (xi) |
| 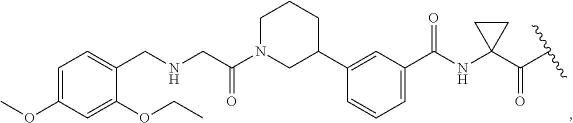 [SMILES: *C(=O)C1(NC(=O)c2cccc(C3CCCN(C(=O)CNCc4ccc(OC)cc4OCC)C3)c2)CC1] | (xj) |
| 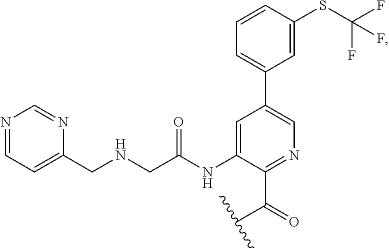 [SMILES: *C(=O)c1ncc(-c2cccc(SC(F)(F)F)c2)cc1NC(=O)CNCc1ccncn1] | (xk) |
| 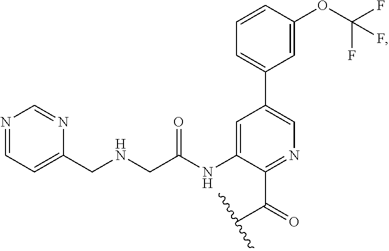 [SMILES: *C(=O)c1ncc(-c2cccc(OC(F)(F)F)c2)cc1NC(=O)CNCc1ccncn1] | (xl) |
| 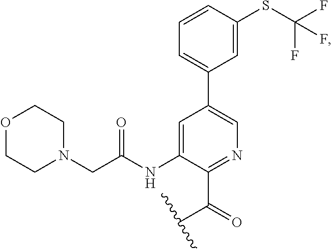 [SMILES: *C(=O)c1ncc(-c2cccc(SC(F)(F)F)c2)cc1NC(=O)CN1CCOCC1] | (xm) |
| 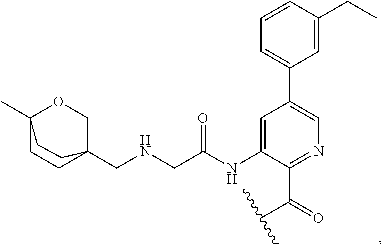 [SMILES: *C(=O)c1ncc(-c2cccc(CC)c2)cc1NC(=O)CNCC12CCC(C)(CC1)OC2] | (xn) |
| 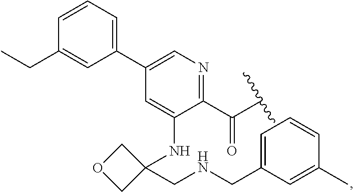 [SMILES: *C(=O)c1ncc(-c2cccc(CC)c2)cc1NC1(CNCc2cccc(C)c2)COC1] | (xo) |
| 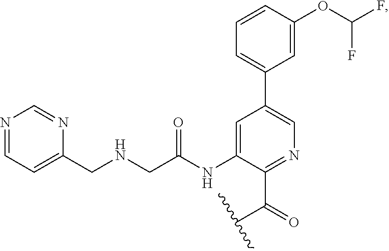 [SMILES: *C(=O)c1ncc(-c2cccc(OC(F)F)c2)cc1NC(=O)CNCc1ccncn1] | (xp) |
| 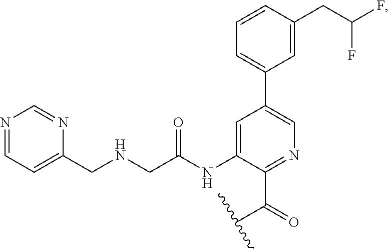 [SMILES: *C(=O)c1ncc(-c2cccc(CC(F)F)c2)cc1NC(=O)CNCc1ccncn1] | (xq) |
| 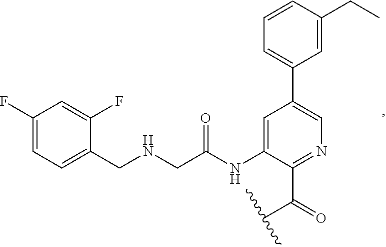 [SMILES: *C(=O)c1ncc(-c2cccc(CC)c2)cc1NC(=O)CNCc1ccc(F)cc1F] | (xr) |
| 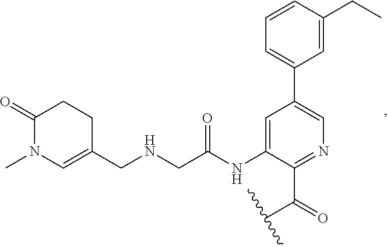 [SMILES: *C(=O)c1ncc(-c2cccc(CC)c2)cc1NC(=O)CNCC1=CN(C)C(=O)CC1] | (xs) |
| 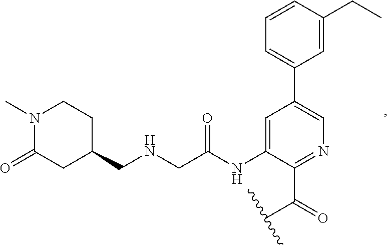 [SMILES: *C(=O)c1ncc(-c2cccc(CC)c2)cc1NC(=O)CNC[C@@H]1CCN(C)C(=O)C1] | (xt) |
| 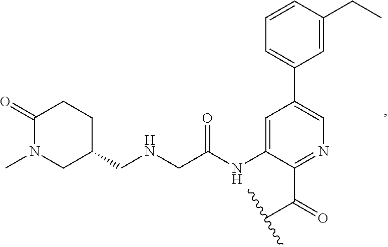 [SMILES: *C(=O)c1ncc(-c2cccc(CC)c2)cc1NC(=O)CNC[C@H]1CCC(=O)N(C)C1] | (xu) |
| 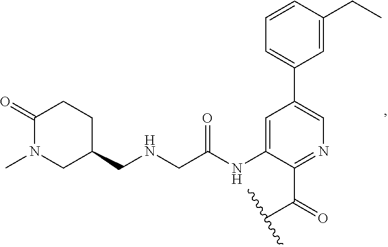 [SMILES: *C(=O)c1ncc(-c2cccc(CC)c2)cc1NC(=O)CNC[C@@H]1CCC(=O)N(C)C1] | (xv) |
| 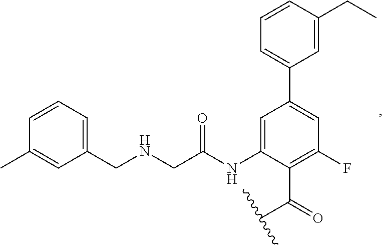 [SMILES: *C(=O)c1c(F)cc(-c2cccc(CC)c2)cc1NC(=O)CNCc1cccc(C)c1] | (xw) |
| 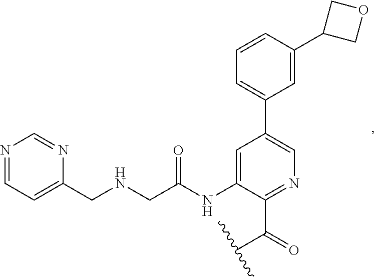 [SMILES: *C(=O)c1ncc(-c2cccc(C3COC3)c2)cc1NC(=O)CNCc1ccncn1] | (xx) |
| 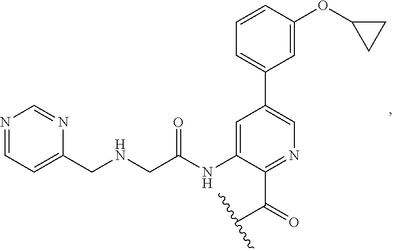 [SMILES: *C(=O)c1ncc(-c2cccc(OC3CC3)c2)cc1NC(=O)CNCc1ccncn1] | (xy) |
| 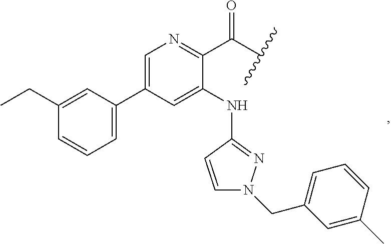 [SMILES: *C(=O)c1ncc(-c2cccc(CC)c2)cc1Nc1ccn(Cc2cccc(C)c2)n1] | (xz) |
| 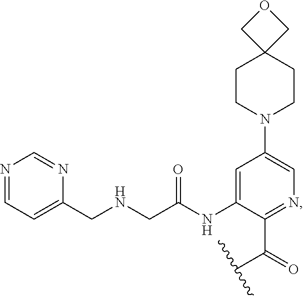 [SMILES: *C(=O)c1ncc(N2CCC3(CC2)COC3)cc1NC(=O)CNCc1ccncn1] | (ya) |
| 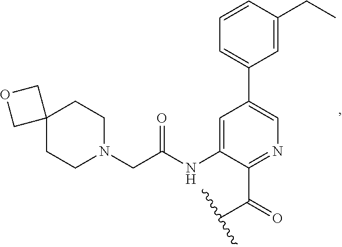 [SMILES: *C(=O)c1ncc(-c2cccc(CC)c2)cc1NC(=O)CN1CCC2(CC1)COC2] | (yb) |
| 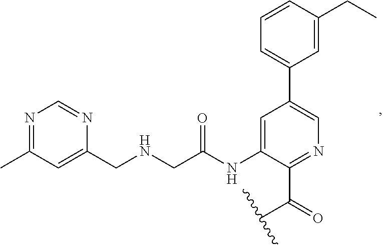 [SMILES: *C(=O)c1ncc(-c2cccc(CC)c2)cc1NC(=O)CNCc1cc(C)ncn1] | (yc) |
| 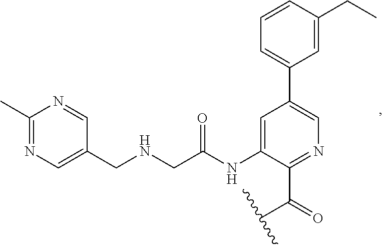 [SMILES: *C(=O)c1ncc(-c2cccc(CC)c2)cc1NC(=O)CNCc1cnc(C)nc1] | (yd) |
| 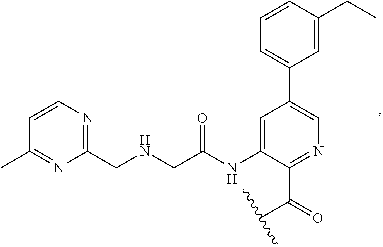 [SMILES: *C(=O)c1ncc(-c2cccc(CC)c2)cc1NC(=O)CNCc1nccc(C)n1] | (ye) |
| 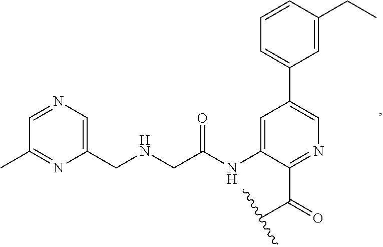 [SMILES: *C(=O)c1ncc(-c2cccc(CC)c2)cc1NC(=O)CNCc1cncc(C)n1] | (yf) |
| 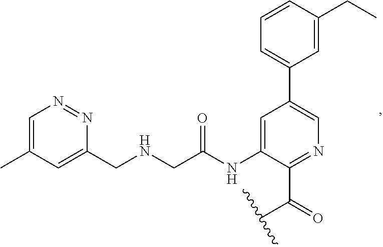 [SMILES: *C(=O)c1ncc(-c2cccc(CC)c2)cc1NC(=O)CNCc1cc(C)cnn1] | (yg) |
| 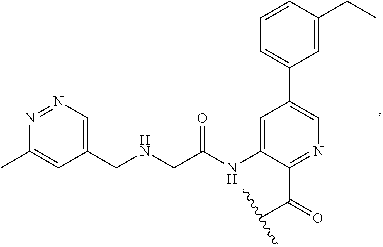 [SMILES: *C(=O)c1ncc(-c2cccc(CC)c2)cc1NC(=O)CNCc1cnnc(C)c1] | (yh) |
| 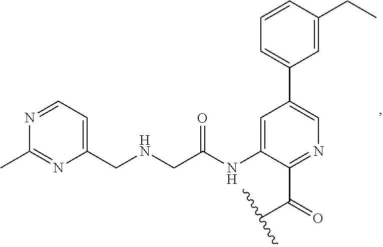 [SMILES: *C(=O)c1ncc(-c2cccc(CC)c2)cc1NC(=O)CNCc1ccnc(C)n1] | (yi) |
| 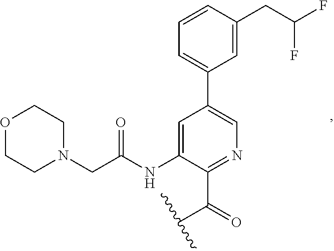 [SMILES: *C(=O)c1ncc(-c2cccc(CC(F)F)c2)cc1NC(=O)CN1CCOCC1] | (yj) |
| 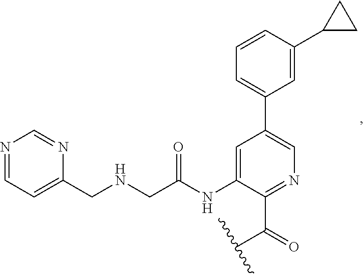 [SMILES: *C(=O)c1ncc(-c2cccc(C3CC3)c2)cc1NC(=O)CNCc1ccncn1] | (yk) |
| 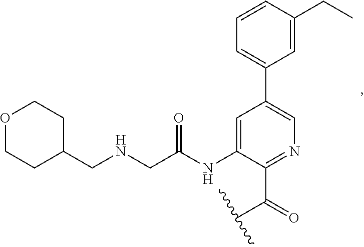 [SMILES: *C(=O)c1ncc(-c2cccc(CC)c2)cc1NC(=O)CNCC1CCOCC1] | (yl) |
| 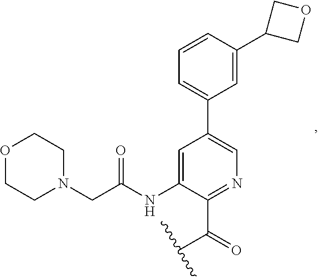 [SMILES: *C(=O)c1ncc(-c2cccc(C3COC3)c2)cc1NC(=O)CN1CCOCC1] | (ym) |
| 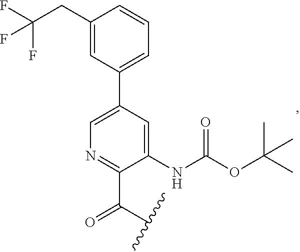 [SMILES: *C(=O)c1ncc(-c2cccc(CC(F)(F)F)c2)cc1NC(=O)OC(C)(C)C] | (yn) |
| 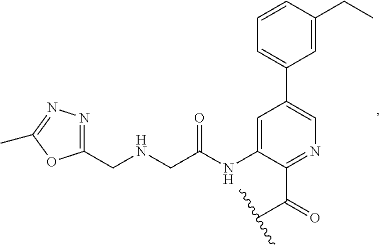 [SMILES: *C(=O)c1ncc(-c2cccc(CC)c2)cc1NC(=O)CNCc1nnc(C)o1] | (yo) |
| 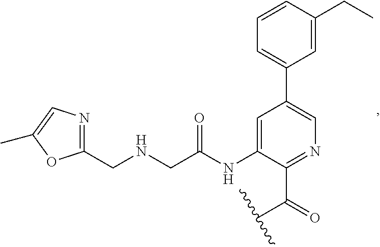 [SMILES: *C(=O)c1ncc(-c2cccc(CC)c2)cc1NC(=O)CNCc1ncc(C)o1] | (yp) |
| 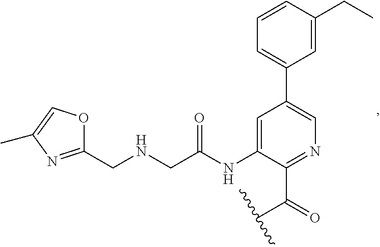 [SMILES: *C(=O)c1ncc(-c2cccc(CC)c2)cc1NC(=O)CNCc1nc(C)co1] | (yq) |
| 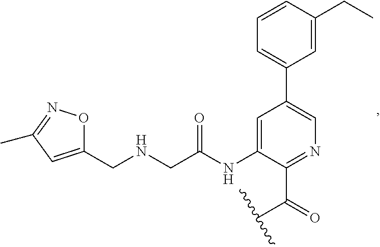 [SMILES: *C(=O)c1ncc(-c2cccc(CC)c2)cc1NC(=O)CNCc1cc(C)no1] | (yr) |
| 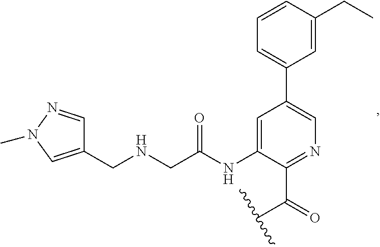 [SMILES: *C(=O)c1ncc(-c2cccc(CC)c2)cc1NC(=O)CNCc1cnn(C)c1] | (ys) |
| 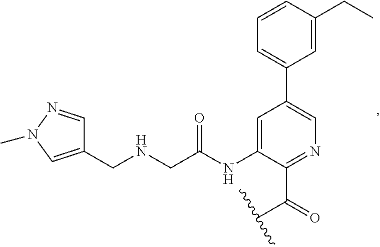 [SMILES: *C(=O)c1ncc(-c2cccc(CC)c2)cc1NC(=O)CNCc1cnn(C)c1] | (yt) |
| 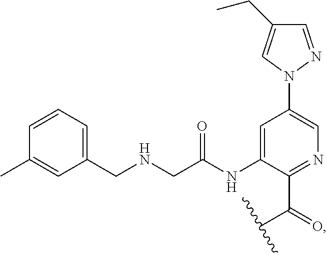 [SMILES: *C(=O)c1ncc(-n2cc(CC)cn2)cc1NC(=O)CNCc1cccc(C)c1] | (yu) |
| 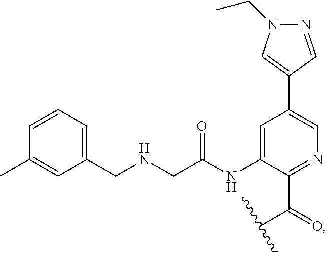 [SMILES: *C(=O)c1ncc(-c2cnn(CC)c2)cc1NC(=O)CNCc1cccc(C)c1] | (yv) |
| 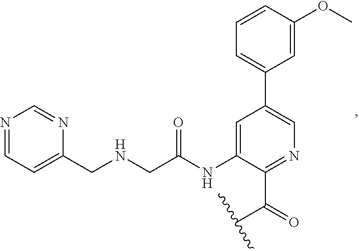 [SMILES: *C(=O)c1ncc(-c2cccc(OC)c2)cc1NC(=O)CNCc1ccncn1] | (yw) |
| 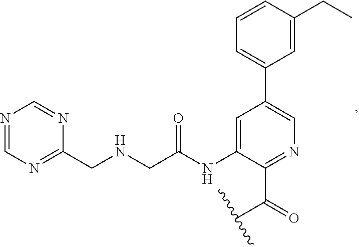 [SMILES: *C(=O)c1ncc(-c2cccc(CC)c2)cc1NC(=O)CNCc1ncncn1] | (yx) |
| 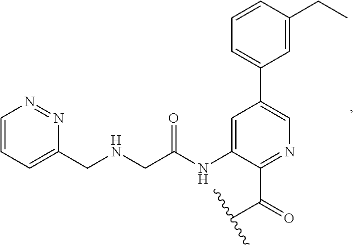 [SMILES: *C(=O)c1ncc(-c2cccc(CC)c2)cc1NC(=O)CNCc1cccnn1] | (yy) |
| 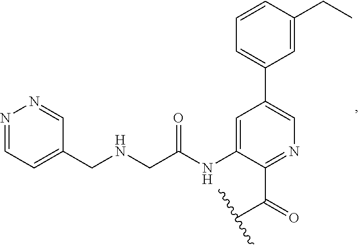 [SMILES: *C(=O)c1ncc(-c2cccc(CC)c2)cc1NC(=O)CNCc1ccnnc1] | (yz) |
| 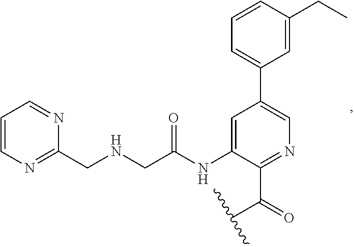 [SMILES: *C(=O)c1ncc(-c2cccc(CC)c2)cc1NC(=O)CNCc1ncccn1] | (za) |
| 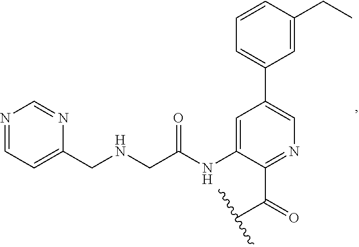 [SMILES: *C(=O)c1ncc(-c2cccc(CC)c2)cc1NC(=O)CNCc1ccncn1] | (zb) |
| 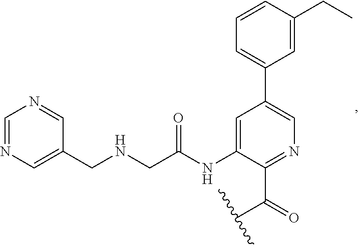 [SMILES: *C(=O)c1ncc(-c2cccc(CC)c2)cc1NC(=O)CNCc1cncnc1] | (zc) |
| 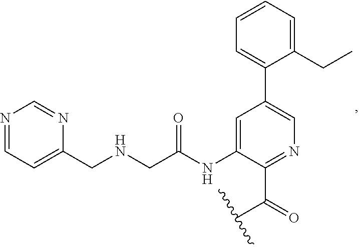 [SMILES: *C(=O)c1ncc(-c2ccccc2CC)cc1NC(=O)CNCc1ccncn1] | (zd) |
| 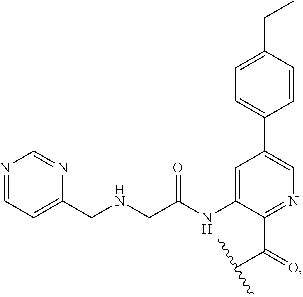 [SMILES: *C(=O)c1ncc(-c2ccc(CC)cc2)cc1NC(=O)CNCc1ccncn1] | (ze) |
| 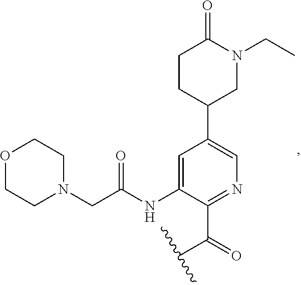 [SMILES: *C(=O)c1ncc(C2CCC(=O)N(CC)C2)cc1NC(=O)CN1CCOCC1] | (zf) |
| 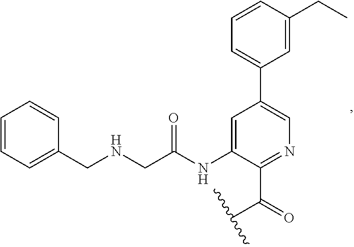 [SMILES: *C(=O)c1ncc(-c2cccc(CC)c2)cc1NC(=O)CNCc1ccccc1] | (zg) |
| 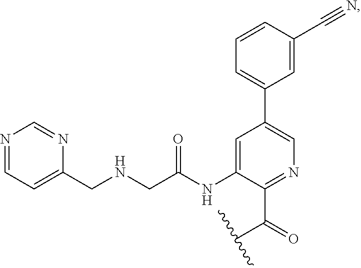 [SMILES: *C(=O)c1ncc(-c2cccc(C#N)c2)cc1NC(=O)CNCc1ccncn1] | (zh) |
| 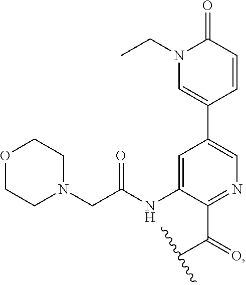 [SMILES: *C(=O)c1ncc(-c2ccc(=O)n(CC)c2)cc1NC(=O)CN1CCOCC1] | (zi) |
| 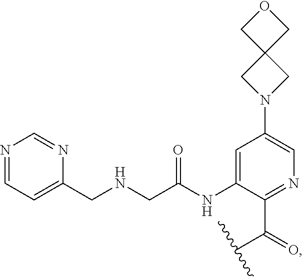 [SMILES: *C(=O)c1ncc(N2CC3(COC3)C2)cc1NC(=O)CNCc1ccncn1] | (zj) |
| 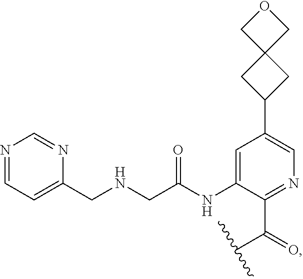 [SMILES: *C(=O)c1ncc(C2CC3(COC3)C2)cc1NC(=O)CNCc1ccncn1] | (zk) |
| 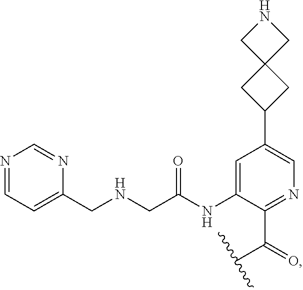 [SMILES: *C(=O)c1ncc(C2CC3(CNC3)C2)cc1NC(=O)CNCc1ccncn1] | (zl) |
| 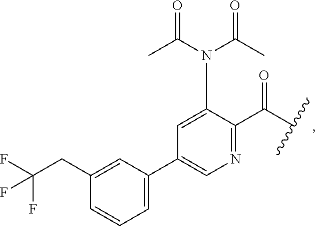 [SMILES: *C(=O)c1ncc(-c2cccc(CC(F)(F)F)c2)cc1N(C(C)=O)C(C)=O] | (zm) |
| 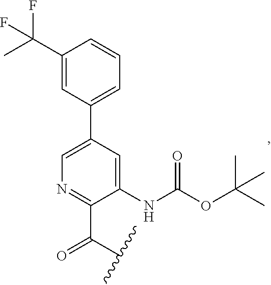 [SMILES: *C(=O)c1ncc(-c2cccc(C(C)(F)F)c2)cc1NC(=O)OC(C)(C)C] | (zn) |
| 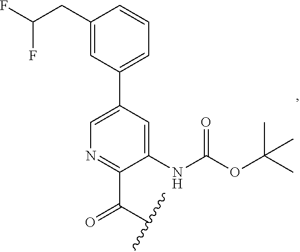 [SMILES: *C(=O)c1ncc(-c2cccc(CC(F)F)c2)cc1NC(=O)OC(C)(C)C] | (zo) |
| 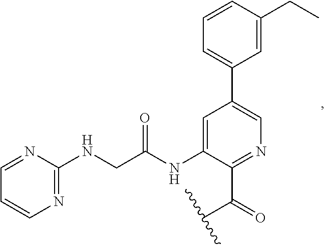 [SMILES: *C(=O)c1ncc(-c2cccc(CC)c2)cc1NC(=O)CNc1ncccn1] | (zp) |
| 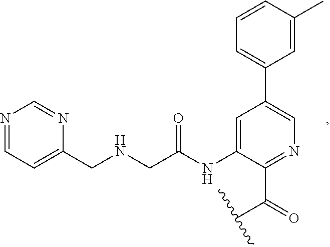 [SMILES: *C(=O)c1ncc(-c2cccc(C)c2)cc1NC(=O)CNCc1ccncn1] | (zq) |
| 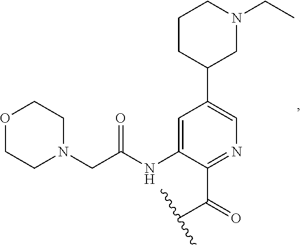 [SMILES: *C(=O)c1ncc(C2CCCN(CC)C2)cc1NC(=O)CN1CCOCC1] | (zr) |
| 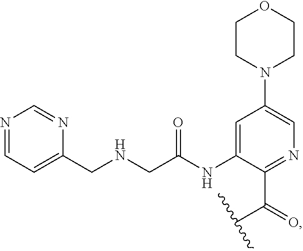 [SMILES: *C(=O)c1ncc(N2CCOCC2)cc1NC(=O)CNCc1ccncn1] | (zs) |
| 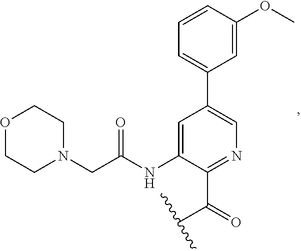 [SMILES: *C(=O)c1ncc(-c2cccc(OC)c2)cc1NC(=O)CN1CCOCC1] | (zt) |
| 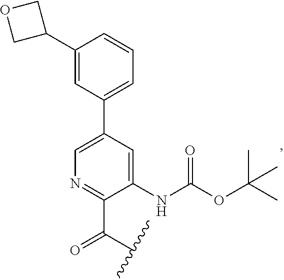 [SMILES: *C(=O)c1ncc(-c2cccc(C3COC3)c2)cc1NC(=O)OC(C)(C)C] | (zu) |
| 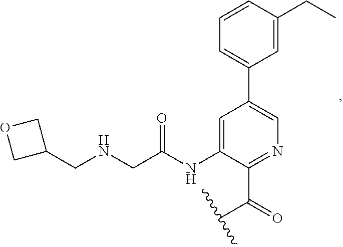 [SMILES: *C(=O)c1ncc(-c2cccc(CC)c2)cc1NC(=O)CNCC1COC1] | (zv) |
| 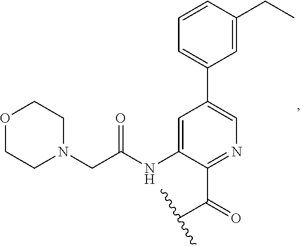 [SMILES: *C(=O)c1ncc(-c2cccc(CC)c2)cc1NC(=O)CN1CCOCC1] | (zw) |
| 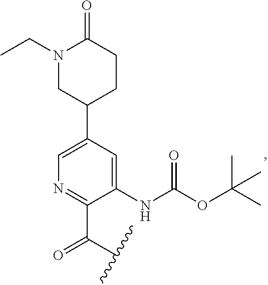 [SMILES: *C(=O)c1ncc(C2CCC(=O)N(CC)C2)cc1NC(=O)OC(C)(C)C] | (zx) |
| 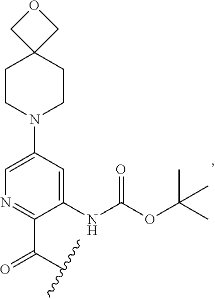 [SMILES: *C(=O)c1ncc(N2CCC3(CC2)COC3)cc1NC(=O)OC(C)(C)C] | (zy) |
| 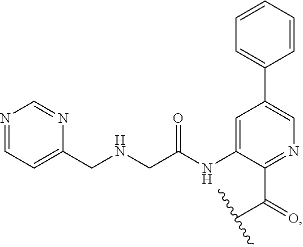 [SMILES: *C(=O)c1ncc(-c2ccccc2)cc1NC(=O)CNCc1ccncn1] | (zz) |
| 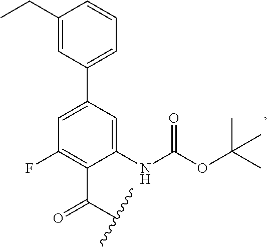 [SMILES: *C(=O)c1c(F)cc(-c2cccc(CC)c2)cc1NC(=O)OC(C)(C)C] | (aaa) |
| 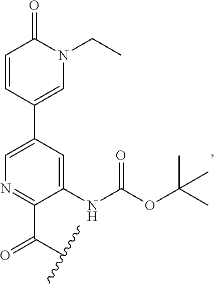 [SMILES: *C(=O)c1ncc(-c2ccc(=O)n(CC)c2)cc1NC(=O)OC(C)(C)C] | (aab) |
| 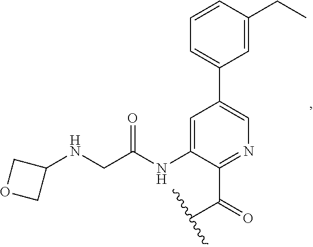 [SMILES: *C(=O)c1ncc(-c2cccc(CC)c2)cc1NC(=O)CNC1COC1] | (aac) |
| 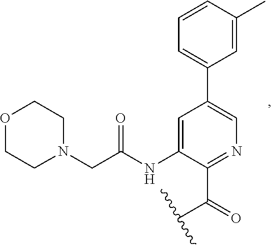 [SMILES: *C(=O)c1ncc(-c2cccc(C)c2)cc1NC(=O)CN1CCOCC1] | (aad) |
| 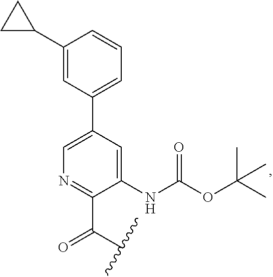 [SMILES: *C(=O)c1ncc(-c2cccc(C3CC3)c2)cc1NC(=O)OC(C)(C)C] | (aae) |
| 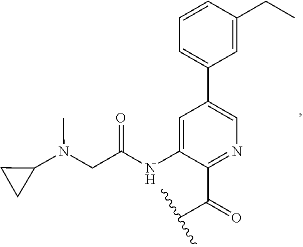 [SMILES: *C(=O)c1ncc(-c2cccc(CC)c2)cc1NC(=O)CN(C)C1CC1] | (aaf) |
| 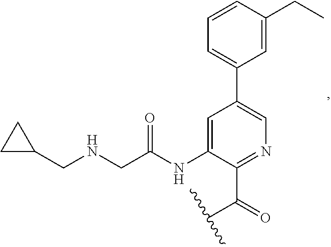 [SMILES: *C(=O)c1ncc(-c2cccc(CC)c2)cc1NC(=O)CNCC1CC1] | (aag) |
| 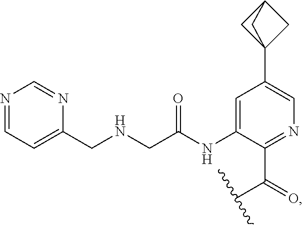 [SMILES: *C(=O)c1ncc(C23CC(C2)C3)cc1NC(=O)CNCc1ccncn1] | (aah) |
| 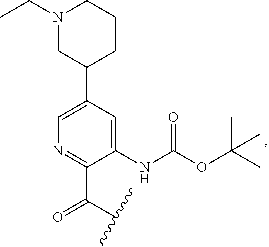 [SMILES: *C(=O)c1ncc(C2CCCN(CC)C2)cc1NC(=O)OC(C)(C)C] | (aai) |
| 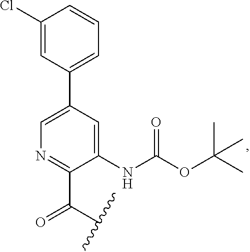 [SMILES: *C(=O)c1ncc(-c2cccc(Cl)c2)cc1NC(=O)OC(C)(C)C] | (aaj) |
| 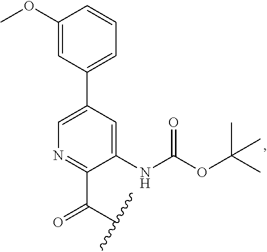 [SMILES: *C(=O)c1ncc(-c2cccc(OC)c2)cc1NC(=O)OC(C)(C)C] | (aak) |
| 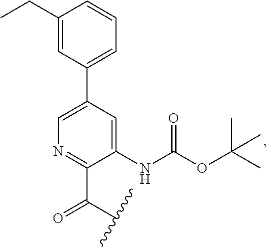 [SMILES: *C(=O)c1ncc(-c2cccc(CC)c2)cc1NC(=O)OC(C)(C)C] | (aal) |
| 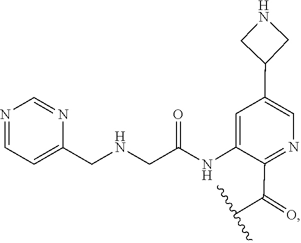 [SMILES: *C(=O)c1ncc(C2CNC2)cc1NC(=O)CNCc1ccncn1] | (aam) |
| 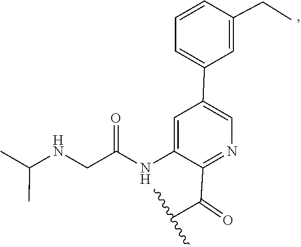 [SMILES: *C(=O)c1ncc(-c2cccc(CC)c2)cc1NC(=O)CNC(C)C] | (aan) |
| 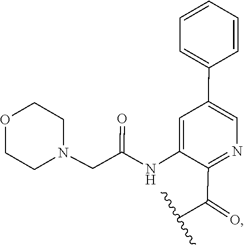 [SMILES: *C(=O)c1ncc(-c2ccccc2)cc1NC(=O)CN1CCOCC1] | (aao) |
| 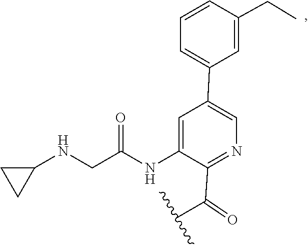 [SMILES: *C(=O)c1ncc(-c2cccc(CC)c2)cc1NC(=O)CNC1CC1] | (aap) |
| 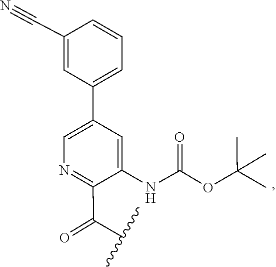 [SMILES: *C(=O)c1ncc(-c2cccc(C#N)c2)cc1NC(=O)OC(C)(C)C] | (aaq) |
| 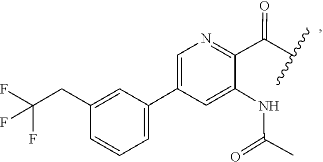 [SMILES: *C(=O)c1ncc(-c2cccc(CC(F)(F)F)c2)cc1NC(C)=O] | (aar) |
| 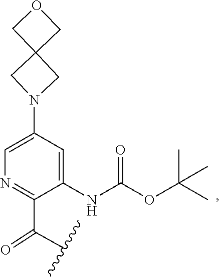 [SMILES: *C(=O)c1ncc(N2CC3(COC3)C2)cc1NC(=O)OC(C)(C)C] | (aas) |
| 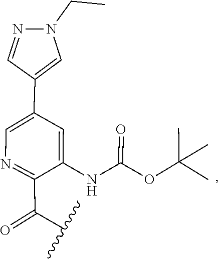 [SMILES: *C(=O)c1ncc(-c2cnn(CC)c2)cc1NC(=O)OC(C)(C)C] | (aat) |
| 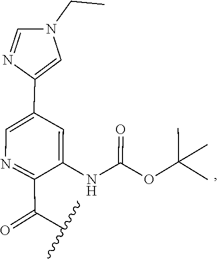 [SMILES: *C(=O)c1ncc(-c2cn(CC)cn2)cc1NC(=O)OC(C)(C)C] | (aau) |
| 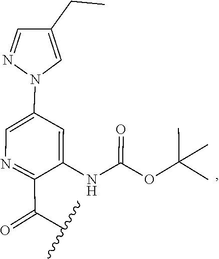 [SMILES: *C(=O)c1ncc(-n2cc(CC)cn2)cc1NC(=O)OC(C)(C)C] | (aav) |
| 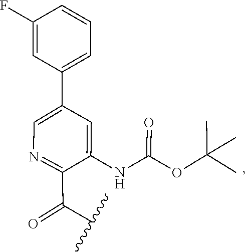 [SMILES: *C(=O)c1ncc(-c2cccc(F)c2)cc1NC(=O)OC(C)(C)C] | (aaw) |
| 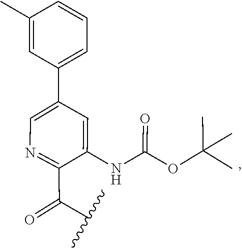 [SMILES: *C(=O)c1ncc(-c2cccc(C)c2)cc1NC(=O)OC(C)(C)C] | (aax) |
| 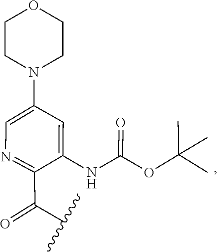 [SMILES: *C(=O)c1ncc(N2CCOCC2)cc1NC(=O)OC(C)(C)C] | (aay) |
| 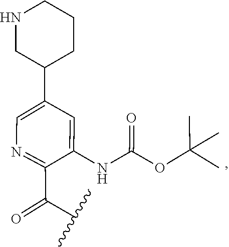 [SMILES: *C(=O)c1ncc(C2CCCNC2)cc1NC(=O)OC(C)(C)C] | (aaz) |
| 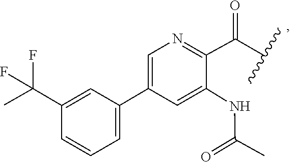 [SMILES: *C(=O)c1ncc(-c2cccc(C(C)(F)F)c2)cc1NC(C)=O] | (aba) |
| 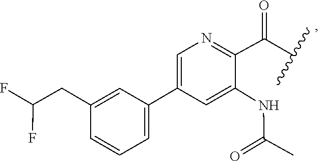 [SMILES: *C(=O)c1ncc(-c2cccc(CC(F)F)c2)cc1NC(C)=O] | (abb) |
| 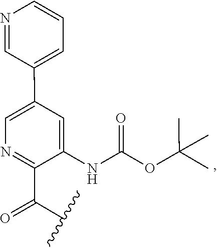 [SMILES: *C(=O)c1ncc(-c2cccnc2)cc1NC(=O)OC(C)(C)C] | (abc) |
| 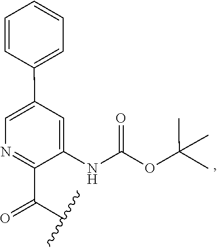 [SMILES: *C(=O)c1ncc(-c2ccccc2)cc1NC(=O)OC(C)(C)C] | (abd) |
| 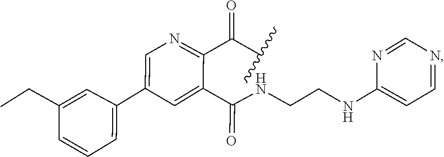 [SMILES: *C(=O)c1ncc(-c2cccc(CC)c2)cc1C(=O)NCCNc1ccncn1] | (abe) |
| 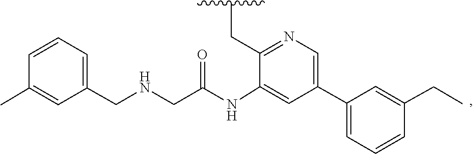 [SMILES: *Cc1ncc(-c2cccc(CC)c2)cc1NC(=O)CNCc1cccc(C)c1] | (abf) |
| 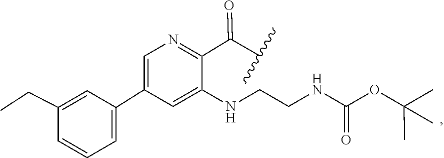 [SMILES: *C(=O)c1ncc(-c2cccc(CC)c2)cc1NCCNC(=O)OC(C)(C)C] | (abg) |
| 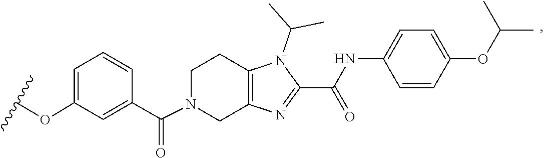 [SMILES: *Oc1cccc(C(=O)N2CCc3c(nc(C(=O)Nc4ccc(OC(C)C)cc4)n3C(C)C)C2)c1] | (abh) |
| 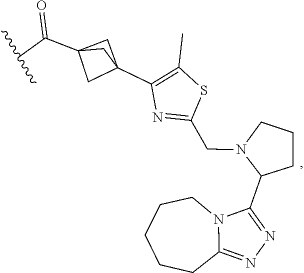 [SMILES: *C(=O)C12CC(c3nc(CN4CCCC4c4nnc5n4CCCCC5)sc3C)(C1)C2] | (abi) |
| 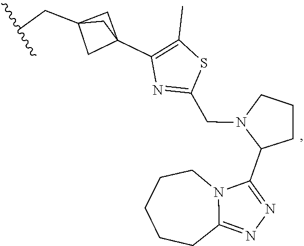 [SMILES: *CC12CC(c3nc(CN4CCCC4c4nnc5n4CCCCC5)sc3C)(C1)C2] | (abj) |
| 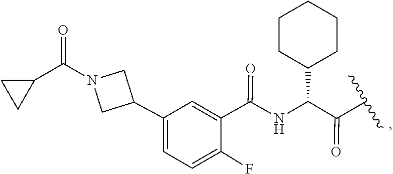 [SMILES: *C(=O)[C@H](NC(=O)c1cc(C2CN(C(=O)C3CC3)C2)ccc1F)C1CCCCC1] | (abk) |
| 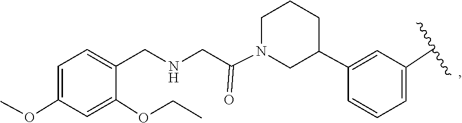 [SMILES: *c1cccc(C2CCCN(C(=O)CNCc3ccc(OC)cc3OCC)C2)c1] | (abl) |
| 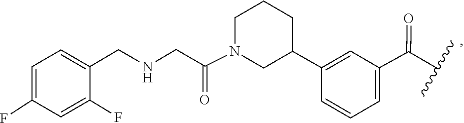 [SMILES: *C(=O)c1cccc(C2CCCN(C(=O)CNCc3ccc(F)cc3F)C2)c1] | (abm) |
| 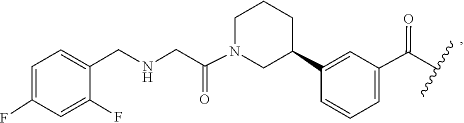 [SMILES: *C(=O)c1cccc([C@@H]2CCCN(C(=O)CNCc3ccc(F)cc3F)C2)c1] | (abn) |
| 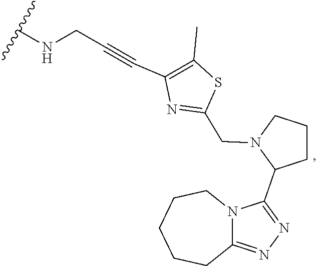 [SMILES: *NCC#Cc1nc(CN2CCCC2c2nnc3n2CCCCC3)sc1C] | (abo) |
| 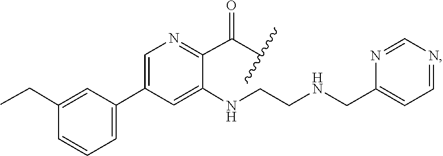 [SMILES: *C(=O)c1ncc(-c2cccc(CC)c2)cc1NCCNCc1ccncn1] | (abp) |
| 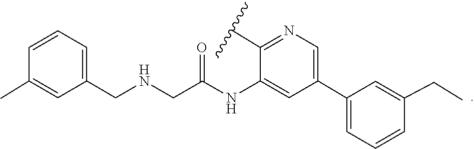 [SMILES: *c1ncc(-c2cccc(CC)c2)cc1NC(=O)CNCc1cccc(C)c1] | (abq) |
| 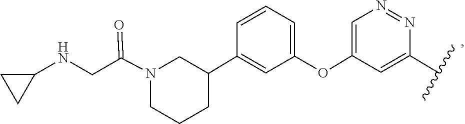 [SMILES: *c1cc(Oc2cccc(C3CCCN(C(=O)CNC4CC4)C3)c2)cnn1] | (abr) |
| 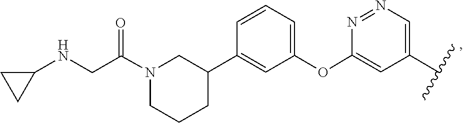 [SMILES: *c1cnnc(Oc2cccc(C3CCCN(C(=O)CNC4CC4)C3)c2)c1] | (abs) |
| 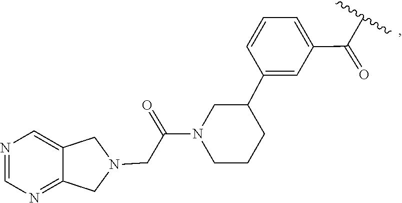 [SMILES: *C(=O)c1cccc(C2CCCN(C(=O)CN3Cc4cncnc4C3)C2)c1] | (abt) |
| 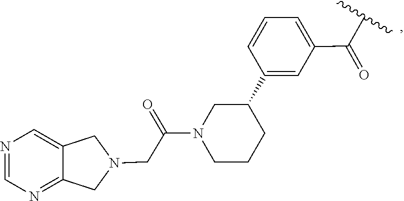 [SMILES: *C(=O)c1cccc([C@@H]2CCCN(C(=O)CN3Cc4cncnc4C3)C2)c1] | (abu) |
| 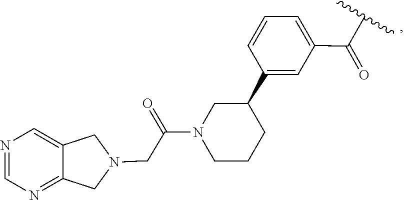 [SMILES: *C(=O)c1cccc([C@H]2CCCN(C(=O)CN3Cc4cncnc4C3)C2)c1] | (abv) |
| 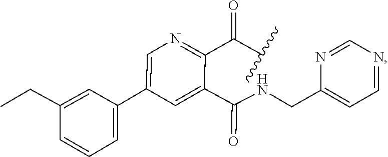 [SMILES: *C(=O)c1ncc(-c2cccc(CC)c2)cc1C(=O)NCc1ccncn1] | (abw) |
| 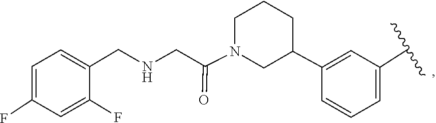 [SMILES: *c1cccc(C2CCCN(C(=O)CNCc3ccc(F)cc3F)C2)c1] | (abx) |
| 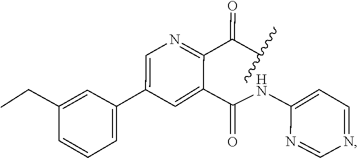 [SMILES: *C(=O)c1ncc(-c2cccc(CC)c2)cc1C(=O)Nc1ccncn1] | (aby) |
| 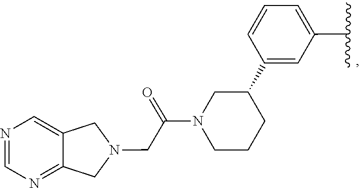 [SMILES: *c1cccc([C@@H]2CCCN(C(=O)CN3Cc4cncnc4C3)C2)c1] | (abz) |
| 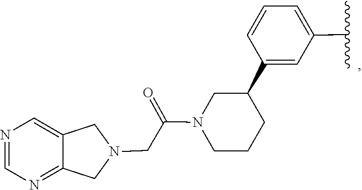 [SMILES: *c1cccc([C@H]2CCCN(C(=O)CN3Cc4cncnc4C3)C2)c1] | (aca) |
| 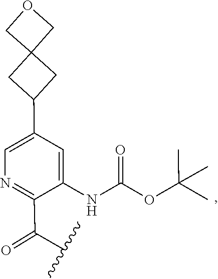 [SMILES: *C(=O)c1ncc(C2CC3(COC3)C2)cc1NC(=O)OC(C)(C)C] | (acb) |
| 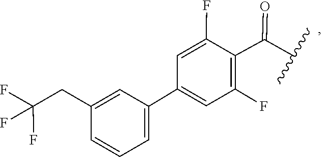 [SMILES: *C(=O)c1c(F)cc(-c2cccc(CC(F)(F)F)c2)cc1F] | (acc) |
| 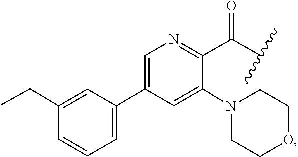 [SMILES: *C(=O)c1ncc(-c2cccc(CC)c2)cc1N1CCOCC1] | (acd) |
| 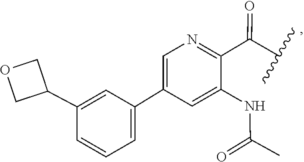 [SMILES: *C(=O)c1ncc(-c2cccc(C3COC3)c2)cc1NC(C)=O] | (ace) |
| 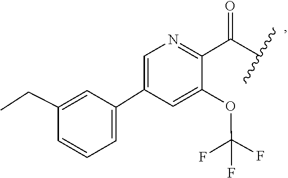 [SMILES: *C(=O)c1ncc(-c2cccc(CC)c2)cc1OC(F)(F)F] | (acf) |
| 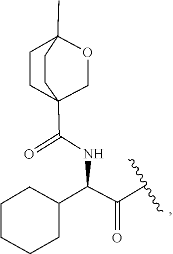 [SMILES: *C(=O)[C@H](NC(=O)C12CCC(C)(CC1)OC2)C1CCCCC1] | (acg) |
| 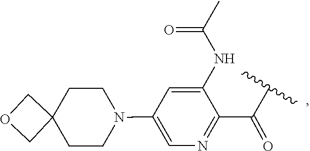 [SMILES: *C(=O)c1ncc(N2CCC3(CC2)COC3)cc1NC(C)=O] | (ach) |
| 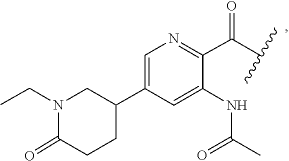 [SMILES: *C(=O)c1ncc(C2CCC(=O)N(CC)C2)cc1NC(C)=O] | (aci) |
| 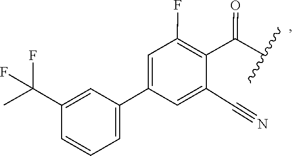 [SMILES: *C(=O)c1c(F)cc(-c2cccc(C(C)(F)F)c2)cc1C#N] | (acj) |
| 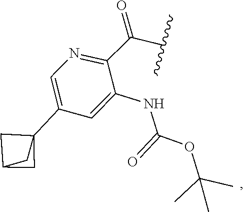 [SMILES: *C(=O)c1ncc(C23CC(C2)C3)cc1NC(=O)OC(C)(C)C] | (ack) |
| 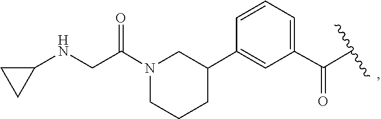 [SMILES: *C(=O)c1cccc(C2CCCN(C(=O)CNC3CC3)C2)c1] | (acl) |
| 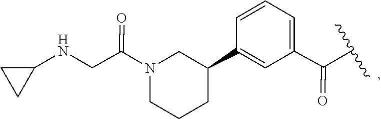 [SMILES: *C(=O)c1cccc([C@H]2CCCN(C(=O)CNC3CC3)C2)c1] | (acm) |
| 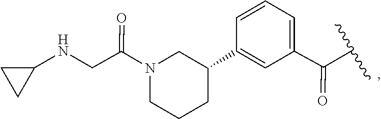 [SMILES: *C(=O)c1cccc([C@@H]2CCCN(C(=O)CNC3CC3)C2)c1] | (acn) |
| 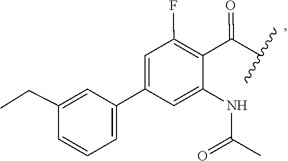 [SMILES: *C(=O)c1c(F)cc(-c2cccc(CC)c2)cc1NC(C)=O] | (aco) |
| 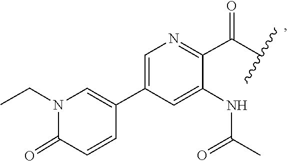 [SMILES: *C(=O)c1ncc(-c2ccc(=O)n(CC)c2)cc1NC(C)=O] | (acp) |
| 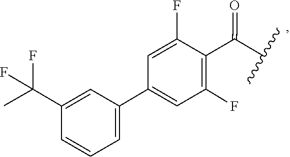 [SMILES: *C(=O)c1c(F)cc(-c2cccc(C(C)(F)F)c2)cc1F] | (acq) |
| 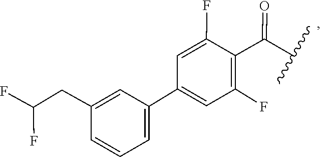 [SMILES: *C(=O)c1c(F)cc(-c2cccc(CC(F)F)c2)cc1F] | (acr) |
| 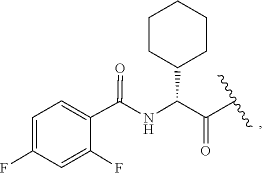 [SMILES: *C(=O)[C@H](NC(=O)c1ccc(F)cc1F)C1CCCCC1] | (acs) |
| 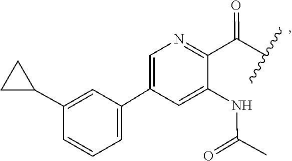 [SMILES: *C(=O)c1ncc(-c2cccc(C3CC3)c2)cc1NC(C)=O] | (act) |
| 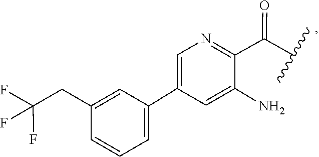 [SMILES: *C(=O)c1ncc(-c2cccc(CC(F)(F)F)c2)cc1N] | (acu) |
| 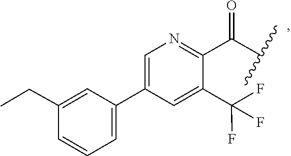 [SMILES: *C(=O)c1ncc(-c2cccc(CC)c2)cc1C(F)(F)F] | (acv) |
| 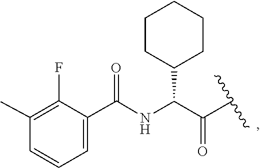 [SMILES: *C(=O)[C@H](NC(=O)c1cccc(C)c1F)C1CCCCC1] | (acw) |
| 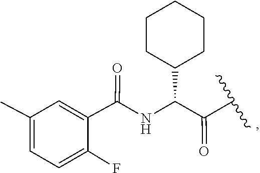 [SMILES: *C(=O)[C@H](NC(=O)c1cc(C)ccc1F)C1CCCCC1] | (acx) |
| 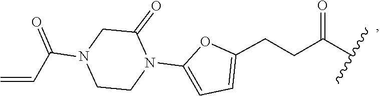 [SMILES: *C(=O)CCc1ccc(N2CCN(C(=O)C=C)CC2=O)o1] | (acy) |
| 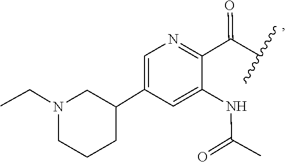 [SMILES: *C(=O)c1ncc(C2CCCN(CC)C2)cc1NC(C)=O] | (acz) |
| 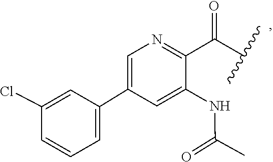 [SMILES: *C(=O)c1ncc(-c2cccc(Cl)c2)cc1NC(C)=O] | (ada) |
| 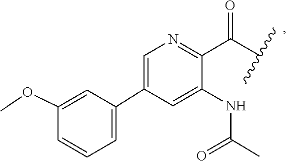 [SMILES: *C(=O)c1ncc(-c2cccc(OC)c2)cc1NC(C)=O] | (adb) |
| 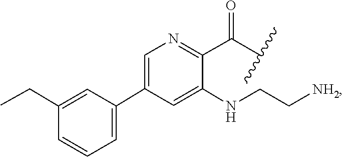 [SMILES: *C(=O)c1ncc(-c2cccc(CC)c2)cc1NCCN] | (adc) |
| 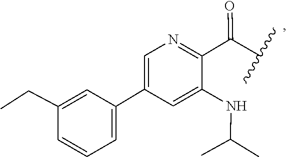 [SMILES: *C(=O)c1ncc(-c2cccc(CC)c2)cc1NC(C)C] | (add) |
| 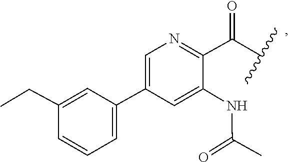 [SMILES: *C(=O)c1ncc(-c2cccc(CC)c2)cc1NC(C)=O] | (ade) |
| 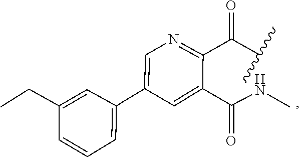 [SMILES: *C(=O)c1ncc(-c2cccc(CC)c2)cc1C(=O)NC] | (adf) |
| 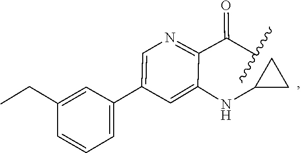 [SMILES: *C(=O)c1ncc(-c2cccc(CC)c2)cc1NC1CC1] | (adg) |
| 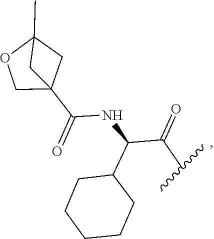 [SMILES: *C(=O)[C@H](NC(=O)C12COC(C)(C1)C2)C1CCCCC1] | (adh) |
| 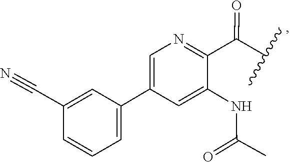 [SMILES: *C(=O)c1ncc(-c2cccc(C#N)c2)cc1NC(C)=O] | (adi) |
| 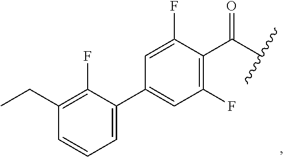 [SMILES: *C(=O)c1c(F)cc(-c2cccc(CC)c2F)cc1F] | (adj) |
| 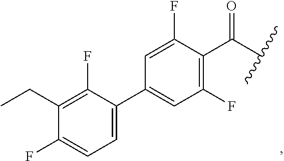 [SMILES: *C(=O)c1c(F)cc(-c2ccc(F)c(CC)c2F)cc1F] | (adk) |
| 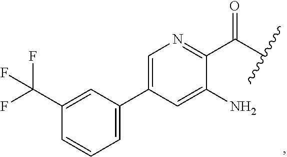 [SMILES: *C(=O)c1ncc(-c2cccc(C(F)(F)F)c2)cc1N] | (adl) |
| 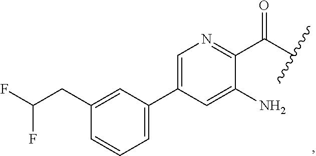 [SMILES: *C(=O)c1ncc(-c2cccc(CC(F)F)c2)cc1N] | (adm) |
| 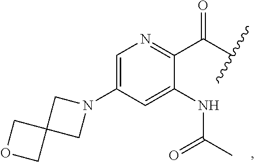 [SMILES: *C(=O)c1ncc(N2CC3(COC3)C2)cc1NC(C)=O] | (adn) |
| 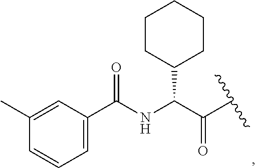 [SMILES: *C(=O)[C@H](NC(=O)c1cccc(C)c1)C1CCCCC1] | (ado) |
| 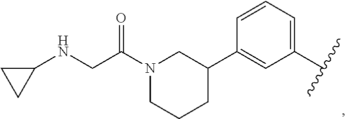 [SMILES: *c1cccc(C2CCCN(C(=O)CNC3CC3)C2)c1] | (adp) |
| 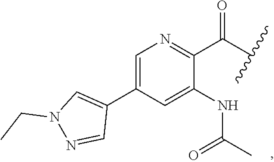 [SMILES: *C(=O)c1ncc(-c2cnn(CC)c2)cc1NC(C)=O] | (adq) |
| 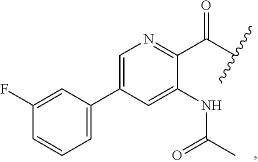 [SMILES: *C(=O)c1ncc(-c2cccc(F)c2)cc1NC(C)=O] | (adr) |
| 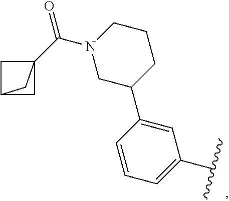 [SMILES: *c1cccc(C2CCCN(C(=O)C34CC(C3)C4)C2)c1] | (ads) |
| 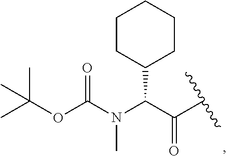 [SMILES: *C(=O)[C@@H](C1CCCCC1)N(C)C(=O)OC(C)(C)C] | (adt) |
| 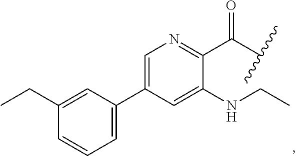 [SMILES: *C(=O)c1ncc(-c2cccc(CC)c2)cc1NCC] | (adu) |
| 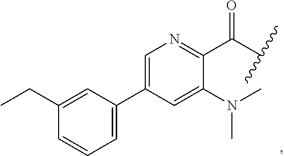 [SMILES: *C(=O)c1ncc(-c2cccc(CC)c2)cc1N(C)C] | (adv) |
| 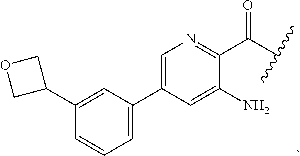 [SMILES: *C(=O)c1ncc(-c2cccc(C3COC3)c2)cc1N] | (adw) |
| 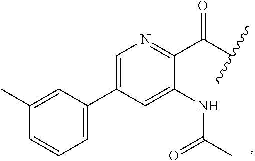 [SMILES: *C(=O)c1ncc(-c2cccc(C)c2)cc1NC(C)=O] | (adx) |
| 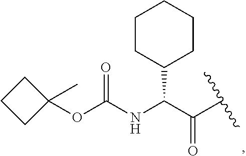 [SMILES: *C(=O)[C@H](NC(=O)OC1(C)CCC1)C1CCCCC1] | (ady) |
| 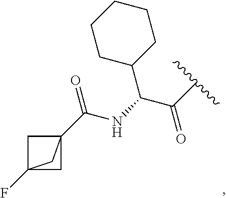 [SMILES: *C(=O)[C@H](NC(=O)C12CC(F)(C1)C2)C1CCCCC1] | (adz) |
| 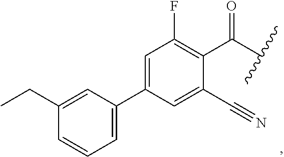 [SMILES: *C(=O)c1c(F)cc(-c2cccc(CC)c2)cc1C#N] | (aea) |
| 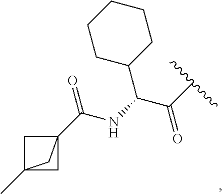 [SMILES: *C(=O)[C@H](NC(=O)C12CC(C)(C1)C2)C1CCCCC1] | (aeb) |
| 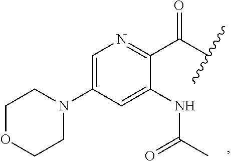 [SMILES: *C(=O)c1ncc(N2CCOCC2)cc1NC(C)=O] | (aec) |
| 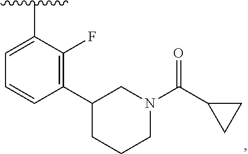 [SMILES: *c1cccc(C2CCCN(C(=O)C3CC3)C2)c1F] | (aed) |
| 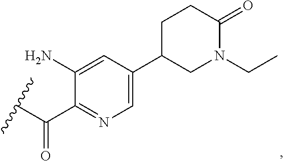 [SMILES: *C(=O)c1ncc(C2CCC(=O)N(CC)C2)cc1N] | (aee) |
| 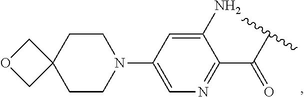 [SMILES: *C(=O)c1ncc(N2CCC3(CC2)COC3)cc1N] | (aef) |
| 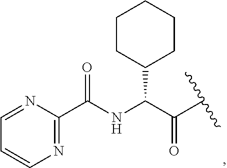 [SMILES: *C(=O)[C@H](NC(=O)c1ncccn1)C1CCCCC1] | (aeg) |
| 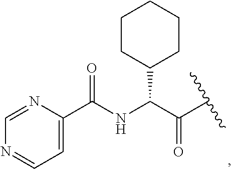 [SMILES: *C(=O)[C@H](NC(=O)c1ccncn1)C1CCCCC1] | (aeh) |
| 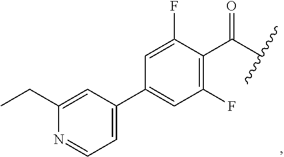 [SMILES: *C(=O)c1c(F)cc(-c2ccnc(CC)c2)cc1F] | (aei) |
| 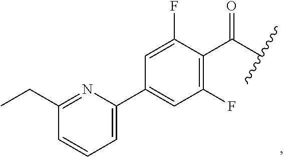 [SMILES: *C(=O)c1c(F)cc(-c2cccc(CC)n2)cc1F] | (aej) |
| 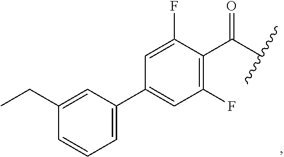 [SMILES: *C(=O)c1c(F)cc(-c2cccc(CC)c2)cc1F] | (aek) |
| 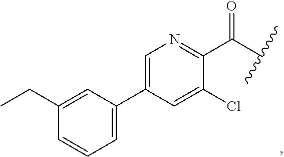 [SMILES: *C(=O)c1ncc(-c2cccc(CC)c2)cc1Cl] | (ael) |
| 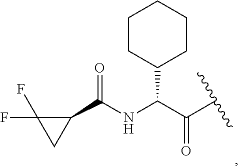 [SMILES: *C(=O)[C@H](NC(=O)[C@H]1CC1(F)F)C1CCCCC1] | (aem) |
| 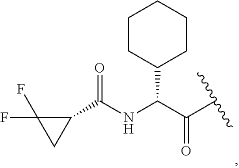 [SMILES: *C(=O)[C@H](NC(=O)[C@@H]1CC1(F)F)C1CCCCC1] | (aen) |
| 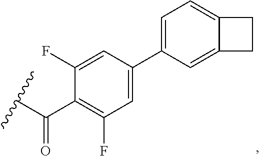 [SMILES: *C(=O)c1c(F)cc(-c2ccc3c(c2)CC3)cc1F] | (aeo) |
| 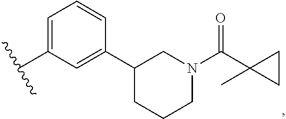 [SMILES: *c1cccc(C2CCCN(C(=O)C3(C)CC3)C2)c1] | (aep) |
| 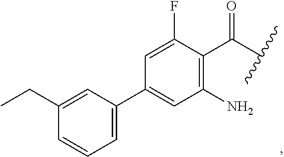 [SMILES: *C(=O)c1c(N)cc(-c2cccc(CC)c2)cc1F] | (aeq) |
| 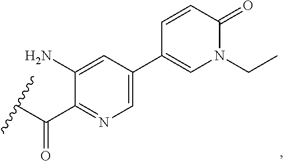 [SMILES: *C(=O)c1ncc(-c2ccc(=O)n(CC)c2)cc1N] | (aer) |
| 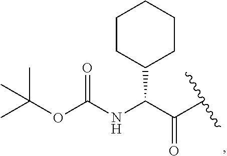 [SMILES: *C(=O)[C@H](NC(=O)OC(C)(C)C)C1CCCCC1] | (aes) |
| 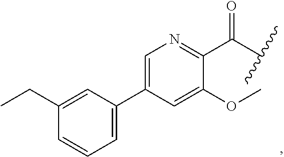 [SMILES: *C(=O)c1ncc(-c2cccc(CC)c2)cc1OC] | (aet) |
| 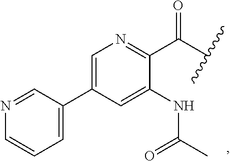 [SMILES: *C(=O)c1ncc(-c2cccnc2)cc1NC(C)=O] | (aeu) |
| 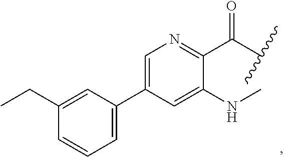 [SMILES: *C(=O)c1ncc(-c2cccc(CC)c2)cc1NC] | (aev) |
| 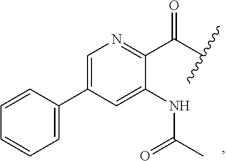 [SMILES: *C(=O)c1ncc(-c2ccccc2)cc1NC(C)=O] | (aew) |
| 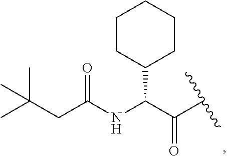 [SMILES: *C(=O)[C@H](NC(=O)CC(C)(C)C)C1CCCCC1] | (aex) |
| 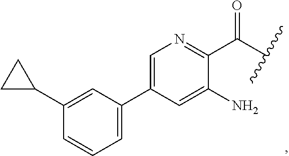 [SMILES: *C(=O)c1ncc(-c2cccc(C3CC3)c2)cc1N] | (aey) |
| 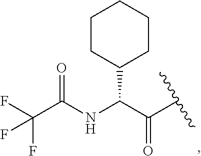 [SMILES: *C(=O)[C@H](NC(=O)C(F)(F)F)C1CCCCC1] | (aez) |
| 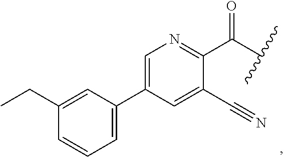 [SMILES: *C(=O)c1ncc(-c2cccc(CC)c2)cc1C#N] | (afa) |
| 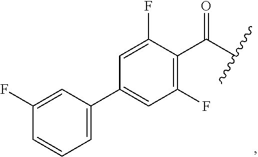 [SMILES: *C(=O)c1c(F)cc(-c2cccc(F)c2)cc1F] | (afb) |
| 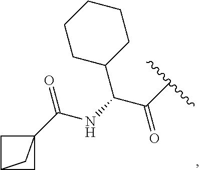 [SMILES: *C(=O)[C@H](NC(=O)C12CC(C1)C2)C1CCCCC1] | (afc) |
| 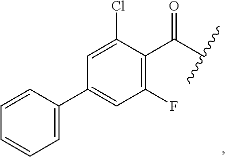 [SMILES: *C(=O)c1c(F)cc(-c2ccccc2)cc1Cl] | (afd) |
| 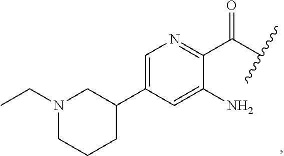 [SMILES: *C(=O)c1ncc(C2CCCN(CC)C2)cc1N] | (afe) |
| 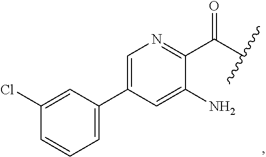 [SMILES: *C(=O)c1ncc(-c2cccc(Cl)c2)cc1N] | (aff) |
| 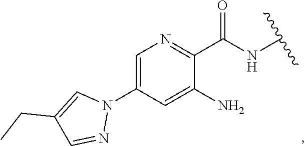 [SMILES: *NC(=O)c1ncc(-n2cc(CC)cn2)cc1N] | (afg) |
| 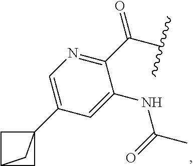 [SMILES: *C(=O)c1ncc(C23CC(C2)C3)cc1NC(C)=O] | (afh) |
| 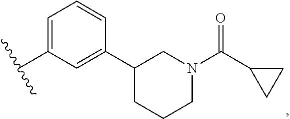 [SMILES: *c1cccc(C2CCCN(C(=O)C3CC3)C2)c1] | (afi) |
| 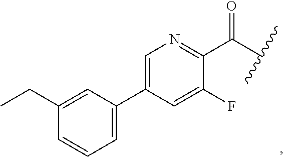 [SMILES: *C(=O)c1ncc(-c2cccc(CC)c2)cc1F] | (afj) |
| 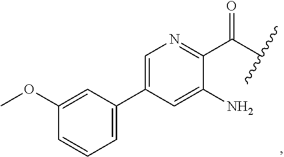 [SMILES: *C(=O)c1ncc(-c2cccc(OC)c2)cc1N] | (afk) |
| 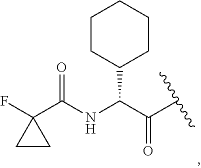 [SMILES: *C(=O)[C@H](NC(=O)C1(F)CC1)C1CCCCC1] | (afl) |
| 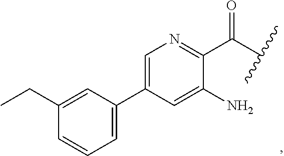 [SMILES: *C(=O)c1ncc(-c2cccc(CC)c2)cc1N] | (afm) |
| 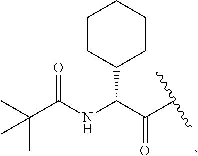 [SMILES: *C(=O)[C@H](NC(=O)C(C)(C)C)C1CCCCC1] | (afn) |
| 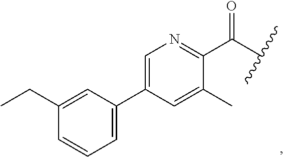 [SMILES: *C(=O)c1ncc(-c2cccc(CC)c2)cc1C] | (afo) |
| 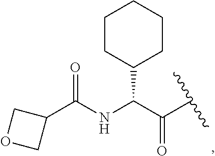 [SMILES: *C(=O)[C@H](NC(=O)C1COC1)C1CCCCC1] | (afp) |
| 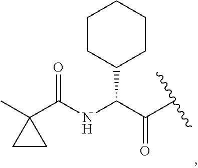 [SMILES: *C(=O)[C@H](NC(=O)C1(C)CC1)C1CCCCC1] | (afq) |
| 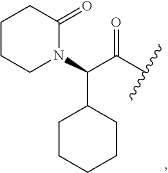 [SMILES: *C(=O)[C@@H](C1CCCCC1)N1CCCCC1=O] | (afr) |
| 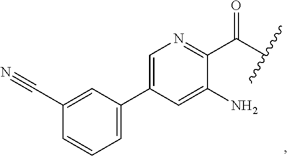 [SMILES: *C(=O)c1ncc(-c2cccc(C#N)c2)cc1N] | (afs) |
| 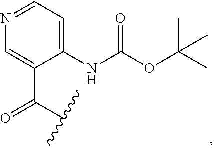 [SMILES: *C(=O)c1cnccc1NC(=O)OC(C)(C)C] | (aft) |
| 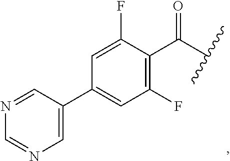 [SMILES: *C(=O)c1c(F)cc(-c2cncnc2)cc1F] | (afu) |
| 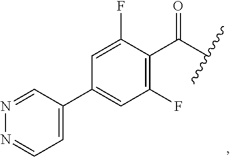 [SMILES: *C(=O)c1c(F)cc(-c2ccnnc2)cc1F] | (afv) |
| 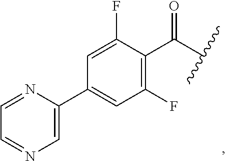 [SMILES: *C(=O)c1c(F)cc(-c2cnccn2)cc1F] | (afw) |
| 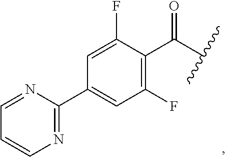 [SMILES: *C(=O)c1c(F)cc(-c2ncccn2)cc1F] | (afx) |
| 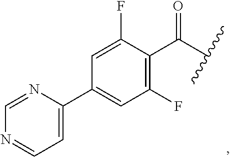 [SMILES: *C(=O)c1c(F)cc(-c2ccncn2)cc1F] | (afy) |
| 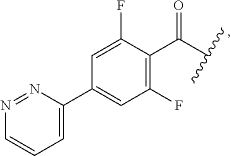 [SMILES: *C(=O)c1c(F)cc(-c2cccnn2)cc1F] | (afz) |
| 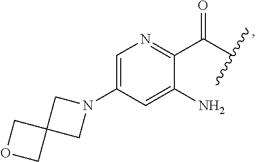 [SMILES: *C(=O)c1ncc(N2CC3(COC3)C2)cc1N] | (aga) |
| 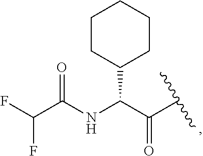 [SMILES: *C(=O)[C@H](NC(=O)C(F)F)C1CCCCC1] | (agb) |
| 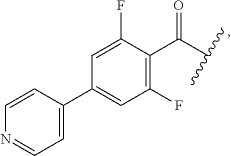 [SMILES: *C(=O)c1c(F)cc(-c2ccncc2)cc1F] | (agc) |
| 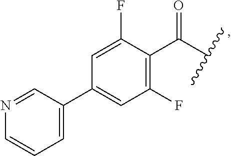 [SMILES: *C(=O)c1c(F)cc(-c2cccnc2)cc1F] | (agd) |
| 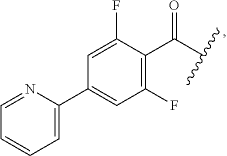 [SMILES: *C(=O)c1c(F)cc(-c2ccccn2)cc1F] | (age) |
| 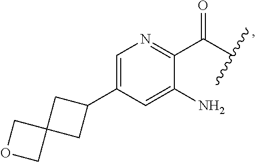 [SMILES: *C(=O)c1ncc(C2CC3(COC3)C2)cc1N] | (agf) |
| 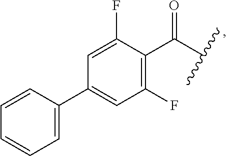 [SMILES: *C(=O)c1c(F)cc(-c2ccccc2)cc1F] | (agg) |
| 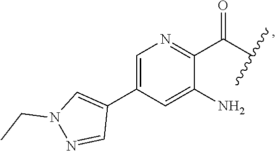 [SMILES: *C(=O)c1ncc(-c2cnn(CC)c2)cc1N] | (agh) |
| 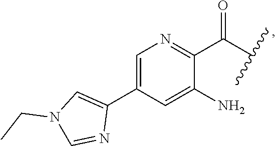 [SMILES: *C(=O)c1ncc(-c2cn(CC)cn2)cc1N] | (agi) |
| 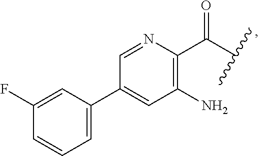 [SMILES: *C(=O)c1ncc(-c2cccc(F)c2)cc1N] | (agj) |
| 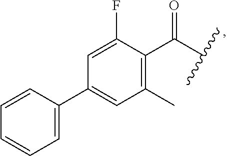 [SMILES: *C(=O)c1c(C)cc(-c2ccccc2)cc1F] | (agk) |
| 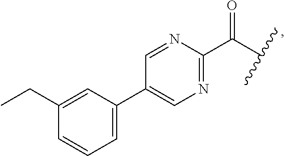 [SMILES: *C(=O)c1ncc(-c2cccc(CC)c2)cn1] | (agl) |
| 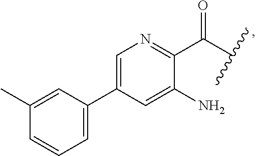 [SMILES: *C(=O)c1ncc(-c2cccc(C)c2)cc1N] | (agm) |
| 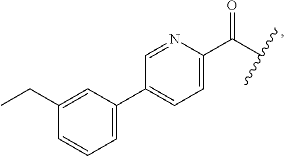 [SMILES: *C(=O)c1ccc(-c2cccc(CC)c2)cn1] | (agn) |
| 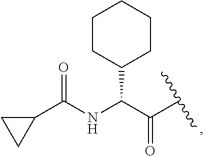 [SMILES: *C(=O)[C@H](NC(=O)C1CC1)C1CCCCC1] | (ago) |
| 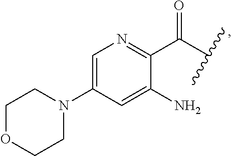 [SMILES: *C(=O)c1ncc(N2CCOCC2)cc1N] | (agp) |
| 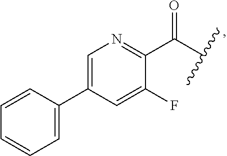 [SMILES: *C(=O)c1ncc(-c2ccccc2)cc1F] | (agq) |
| 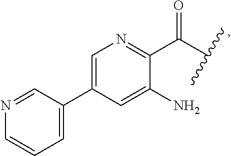 [SMILES: *C(=O)c1ncc(-c2cccnc2)cc1N] | (agr) |
| 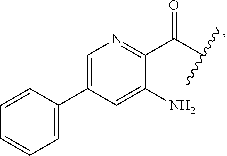 [SMILES: *C(=O)c1ncc(-c2ccccc2)cc1N] | (ags) |
| 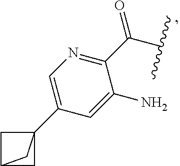 [SMILES: *C(=O)c1ncc(C23CC(C2)C3)cc1N] | (agt) |
| 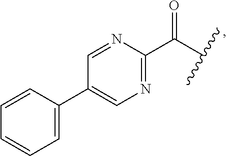 [SMILES: *C(=O)c1ncc(-c2ccccc2)cn1] | (agu) |
| 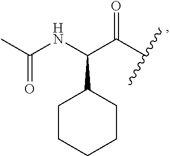 [SMILES: *C(=O)[C@H](NC(C)=O)C1CCCCC1] | (agv) |
| 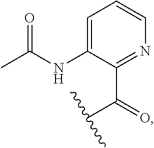 [SMILES: *C(=O)c1ncccc1NC(C)=O] | (agw) |
| 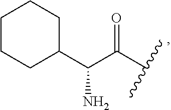 [SMILES: *C(=O)[C@H](N)C1CCCCC1] | (agx) |
| 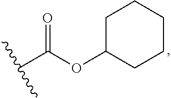 [SMILES: *C(=O)OC1CCCCC1] | (agy) |
| 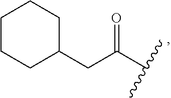 [SMILES: *C(=O)CC1CCCCC1] | (agz) |
| 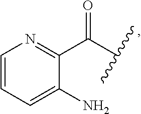 [SMILES: *C(=O)c1ncccc1N] | (aha) |
| 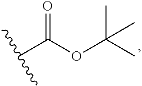 [SMILES: *C(=O)OC(C)(C)C] | (ahb) |
| 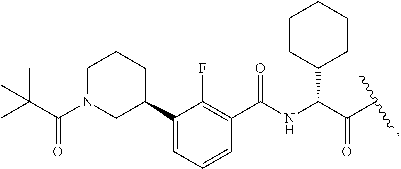 [SMILES: *C(=O)[C@H](NC(=O)c1cccc([C@@H]2CCCN(C(=O)C(C)(C)C)C2)c1F)C1CCCCC1] | (ahc) |
| 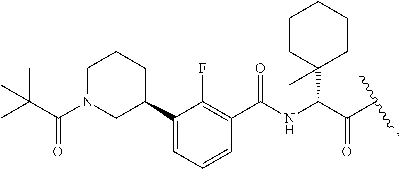 [SMILES: *C(=O)[C@H](NC(=O)c1cccc([C@@H]2CCCN(C(=O)C(C)(C)C)C2)c1F)C1(C)CCCCC1] | (ahd) |
| 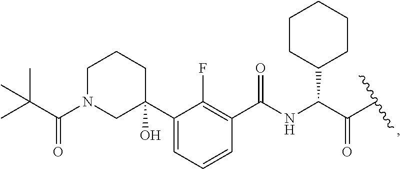 [SMILES: *C(=O)[C@H](NC(=O)c1cccc([C@]2(O)CCCN(C(=O)C(C)(C)C)C2)c1F)C1CCCCC1] | (ahe) |
| 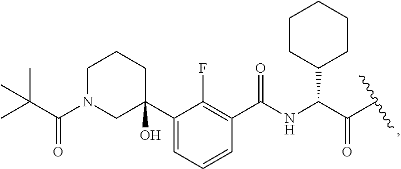 [SMILES: *C(=O)[C@H](NC(=O)c1cccc([C@@]2(O)CCCN(C(=O)C(C)(C)C)C2)c1F)C1CCCCC1] | (ahf) |
| 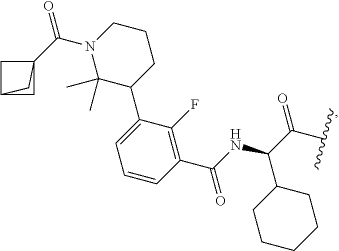 [SMILES: *C(=O)[C@H](NC(=O)c1cccc(C2CCCN(C(=O)C34CC(C3)C4)C2(C)C)c1F)C1CCCCC1] | (ahg) |
| 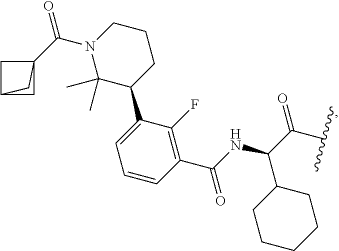 [SMILES: *C(=O)[C@H](NC(=O)c1cccc([C@@H]2CCCN(C(=O)C34CC(C3)C4)C2(C)C)c1F)C1CCCCC1] | (ahh) |
| 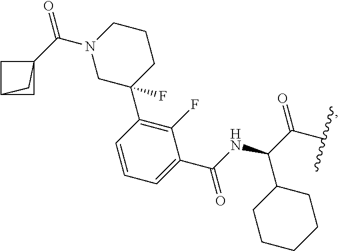 [SMILES: *C(=O)[C@H](NC(=O)c1cccc([C@]2(F)CCCN(C(=O)C34CC(C3)C4)C2)c1F)C1CCCCC1] | (ahi) |
| 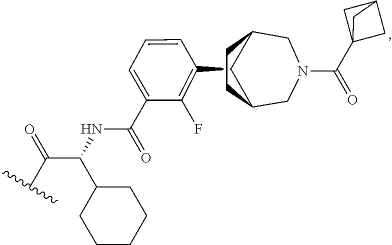 [SMILES: *C(=O)[C@H](NC(=O)c1cccc([C@@H]2[C@@H]3CC[C@H]2CN(C(=O)C24CC(C2)C4)C3)c1F)C1CCCCC1] | (ahj) |
| 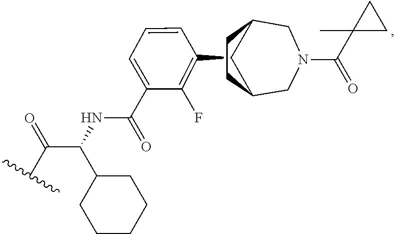 [SMILES: *C(=O)[C@H](NC(=O)c1cccc([C@@H]2[C@@H]3CC[C@H]2CN(C(=O)C2(C)CC2)C3)c1F)C1CCCCC1] | (ahk) |
| 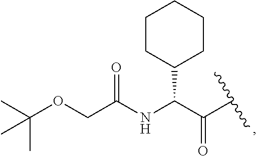 [SMILES: *C(=O)[C@H](NC(=O)COC(C)(C)C)C1CCCCC1] | (ahl) |
| 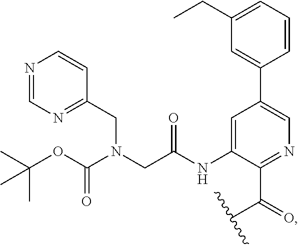 [SMILES: *C(=O)c1ncc(-c2cccc(CC)c2)cc1NC(=O)CN(Cc1ccncn1)C(=O)OC(C)(C)C] | (ahm) |
| 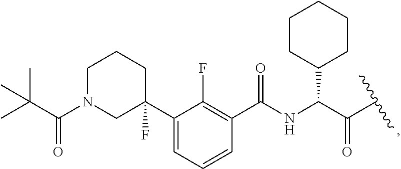 [SMILES: *C(=O)[C@H](NC(=O)c1cccc([C@]2(F)CCCN(C(=O)C(C)(C)C)C2)c1F)C1CCCCC1] | (ahn) |
| 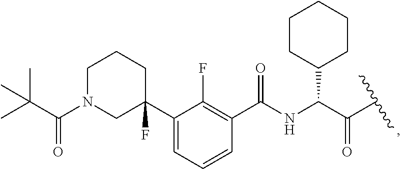 [SMILES: *C(=O)[C@H](NC(=O)c1cccc([C@@]2(F)CCCN(C(=O)C(C)(C)C)C2)c1F)C1CCCCC1] | (aho) |
| 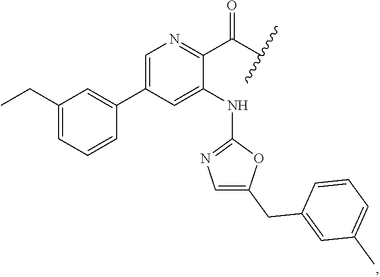 [SMILES: *C(=O)c1ncc(-c2cccc(CC)c2)cc1Nc1ncc(Cc2cccc(C)c2)o1] | (ahp) |
| 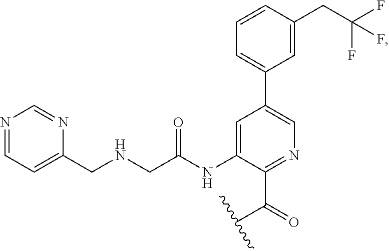 [SMILES: *C(=O)c1ncc(-c2cccc(CC(F)(F)F)c2)cc1NC(=O)CNCc1ccncn1] | (ahq) |
| 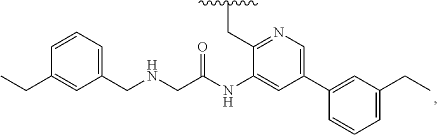 [SMILES: *Cc1ncc(-c2cccc(CC)c2)cc1NC(=O)CNCc1cccc(CC)c1] | (ahr) |
| 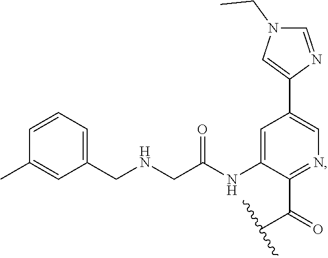 [SMILES: *C(=O)c1ncc(-c2cn(CC)cn2)cc1NC(=O)CNCc1cccc(C)c1] | (ahs) |
| 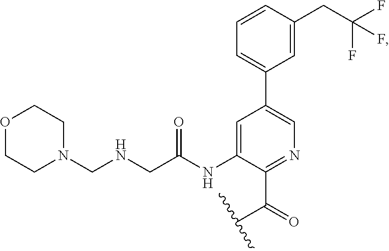 [SMILES: *C(=O)c1ncc(-c2cccc(CC(F)(F)F)c2)cc1NC(=O)CNCN1CCOCC1] | (aht) |
| 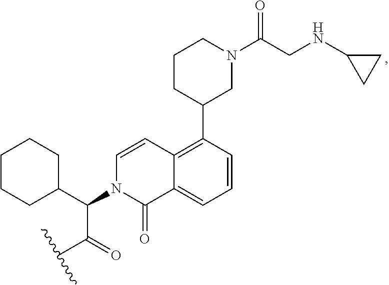 [SMILES: *C(=O)[C@@H](C1CCCCC1)n1ccc2c(C3CCCN(C(=O)CNC4CC4)C3)cccc2c1=O] | (ahu) |
| 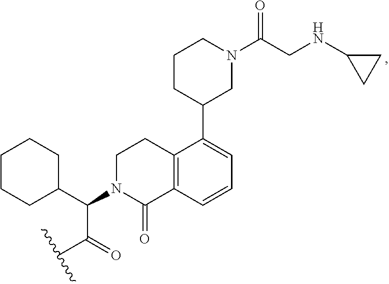 [SMILES: *C(=O)[C@@H](C1CCCCC1)N1CCc2c(cccc2C2CCCN(C(=O)CNC3CC3)C2)C1=O] | (ahv) |
| 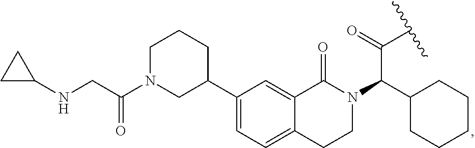 [SMILES: *C(=O)[C@@H](C1CCCCC1)N1CCc2ccc(C3CCCN(C(=O)CNC4CC4)C3)cc2C1=O] | (ahw) |
| 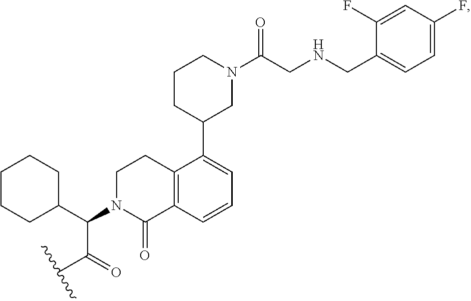 [SMILES: *C(=O)[C@@H](C1CCCCC1)N1CCc2c(cccc2C2CCCN(C(=O)CNCc3ccc(F)cc3F)C2)C1=O] | (ahx) |
| 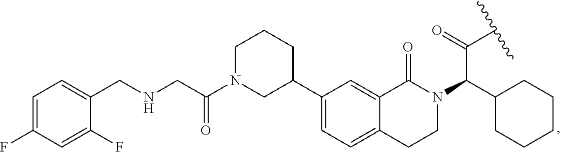 [SMILES: *C(=O)[C@@H](C1CCCCC1)N1CCc2ccc(C3CCCN(C(=O)CNCc4ccc(F)cc4F)C3)cc2C1=O] | (ahy) |
| 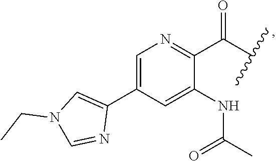 [SMILES: *C(=O)c1ncc(-c2cn(CC)cn2)cc1NC(C)=O] | (ahz) |
| 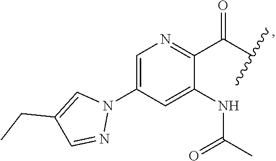 [SMILES: *C(=O)c1ncc(-n2cc(CC)cn2)cc1NC(C)=O] | (aia) |
| 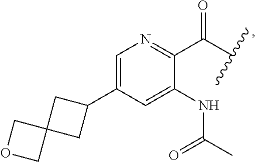 [SMILES: *C(=O)c1ncc(C2CC3(COC3)C2)cc1NC(C)=O] | (aib) |
| 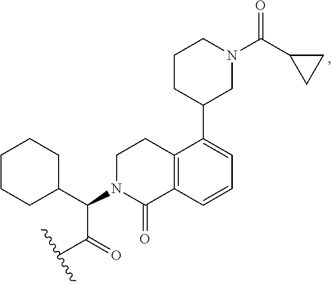 [SMILES: *C(=O)[C@@H](C1CCCCC1)N1CCc2c(cccc2C2CCCN(C(=O)C3CC3)C2)C1=O] | (aic) |
| 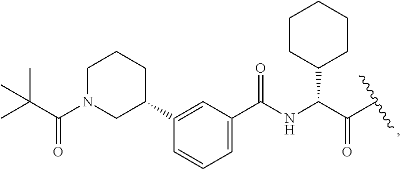 [SMILES: *C(=O)[C@H](NC(=O)c1cccc([C@H]2CCCN(C(=O)C(C)(C)C)C2)c1)C1CCCCC1] | (aid) |
| 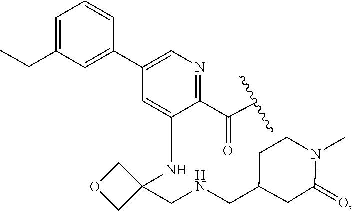 [SMILES: *C(=O)c1ncc(-c2cccc(CC)c2)cc1NC1(CNCC2CCN(C)C(=O)C2)COC1] | (aie) |
| 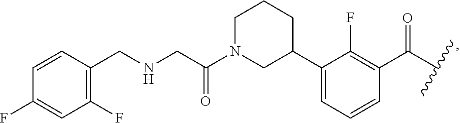 [SMILES: *C(=O)c1cccc(C2CCCN(C(=O)CNCc3ccc(F)cc3F)C2)c1F] | (aif) |
| 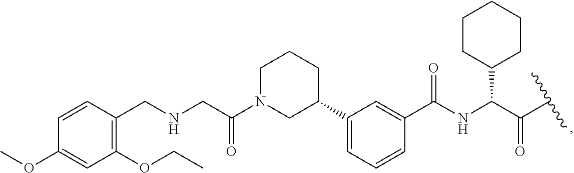 [SMILES: *C(=O)[C@H](NC(=O)c1cccc([C@H]2CCCN(C(=O)CNCc3ccc(OC)cc3OCC)C2)c1)C1CCCCC1] | (aig) |
| 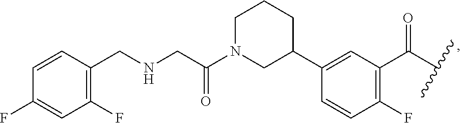 [SMILES: *C(=O)c1cc(C2CCCN(C(=O)CNCc3ccc(F)cc3F)C2)ccc1F] | (aih) |
| 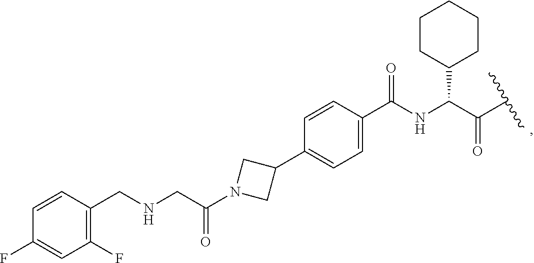 [SMILES: *C(=O)[C@H](NC(=O)c1ccc(C2CN(C(=O)CNCc3ccc(F)cc3F)C2)cc1)C1CCCCC1] | (aii) |
| 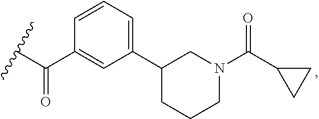 [SMILES: *C(=O)c1cccc(C2CCCN(C(=O)C3CC3)C2)c1] | (aij) |
| 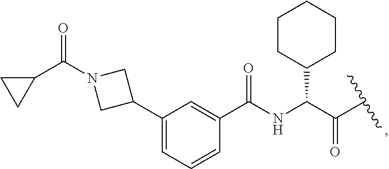 [SMILES: *C(=O)[C@H](NC(=O)c1cccc(C2CN(C(=O)C3CC3)C2)c1)C1CCCCC1] | (aik) |
| 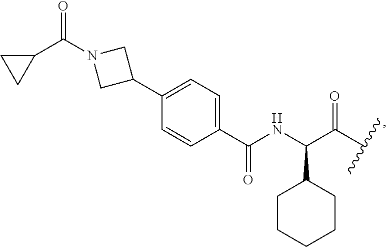 [SMILES: *C(=O)[C@H](NC(=O)c1ccc(C2CN(C(=O)C3CC3)C2)cc1)C1CCCCC1] | (ail) |
| 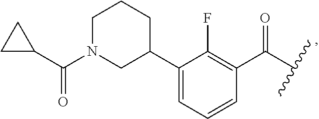 [SMILES: *C(=O)c1cccc(C2CCCN(C(=O)C3CC3)C2)c1F] | (aim) |
| 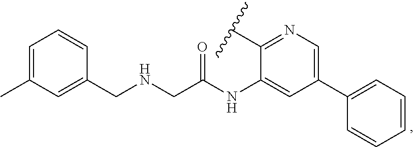 [SMILES: *c1ncc(-c2ccccc2)cc1NC(=O)CNCc1cccc(C)c1] | (ain) |
| 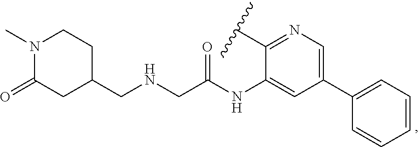 [SMILES: *c1ncc(-c2ccccc2)cc1NC(=O)CNCC1CCN(C)C(=O)C1] | (aio) |
| 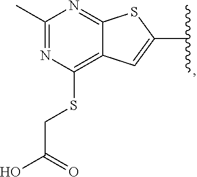 [SMILES: *c1cc2c(SCC(=O)O)nc(C)nc2s1] | (aip) |
| 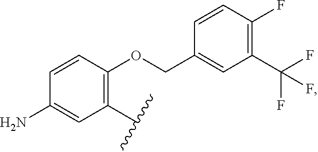 [SMILES: *c1cc(N)ccc1OCc1ccc(F)c(C(F)(F)F)c1] | (aiq) |
| 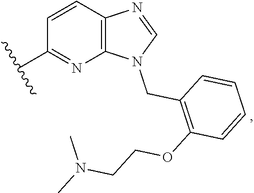 [SMILES: *c1ccc2ncn(Cc3ccccc3OCCN(C)C)c2n1] | (air) |
| 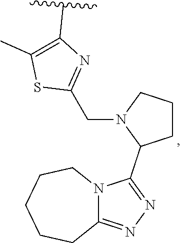 [SMILES: *c1nc(CN2CCCC2c2nnc3n2CCCCC3)sc1C] | (ais) |
| 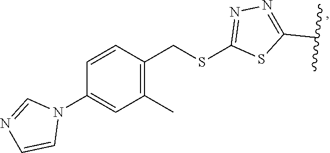 [SMILES: *c1nnc(SCc2ccc(-n3ccnc3)cc2C)s1] | (ait) |
| 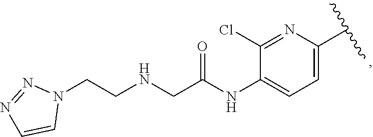 [SMILES: *c1ccc(NC(=O)CNCCn2ccnn2)c(Cl)n1] | (aiu) |
| 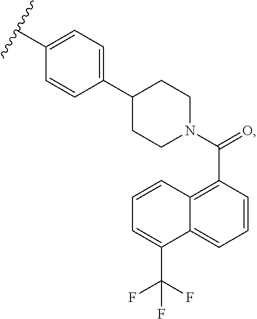 [SMILES: *c1ccc(C2CCN(C(=O)c3cccc4c(C(F)(F)F)cccc34)CC2)cc1] | (aiv) |
| 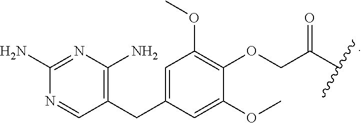 [SMILES: *C(=O)COc1c(OC)cc(Cc2cnc(N)nc2N)cc1OC] | (aiw) |

TABLE B
Exemplified Linkers (L)

| | |
|---|---|
| 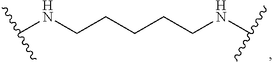 [SMILES: *NCCCCCN*] | (1) |
| 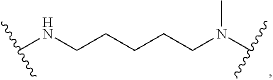 [SMILES: *NCCCCCN(*)C] | (2) |
| 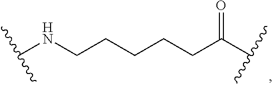 [SMILES: *NCCCCCC(*)=O] | (3) |
| 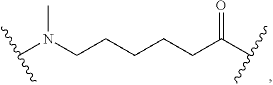 [SMILES: *C(=O)CCCCCN(*)C] | (4) |
| 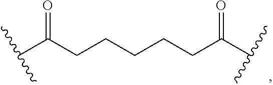 [SMILES: *C(=O)CCCCCC(*)=O] | (5) |
| 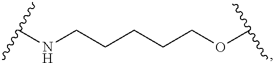 [SMILES: *NCCCCCO*] | (6) |
| 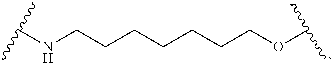 [SMILES: *NCCCCCCCO*] | (7) |
| 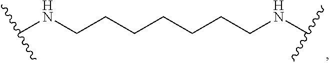 [SMILES: *NCCCCCCCN*] | (8) |
| 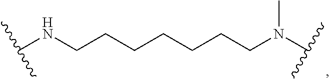 [SMILES: *NCCCCCCCN(*)C] | (9) |
| 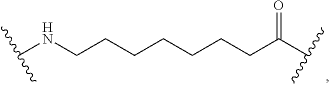 [SMILES: *NCCCCCCCC(*)=O] | (10) |
| 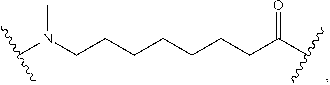 [SMILES: *C(=O)CCCCCCCN(*)C] | (11) |
| 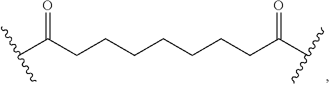 [SMILES: *C(=O)CCCCCCCC(*)=O] | (12) |
| 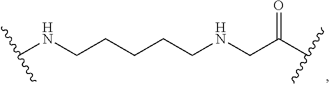 [SMILES: *NCCCCCNCC(*)=O] | (13) |
| 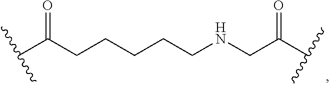 [SMILES: *C(=O)CCCCCNCC(*)=O] | (14) |
| 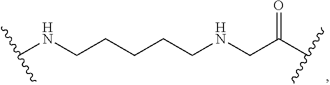 [SMILES: *NCCCCCNCC(*)=O] | (15) |
| 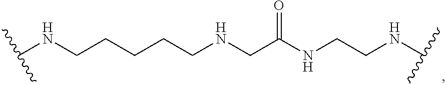 [SMILES: *NCCCCCNCC(=O)NCCN*] | (16) |
| 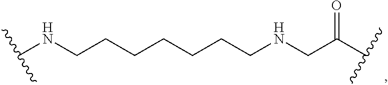 [SMILES: *NCCCCCCCNCC(*)=O] | (17) |
| 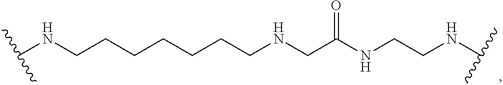 [SMILES: *NCCCCCCCNCC(=O)NCCN*] | (18) |
| 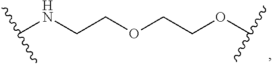 [SMILES: *NCCOCCO*] | (19) |
| 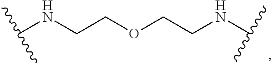 [SMILES: *NCCOCCN*] | (20) |
| 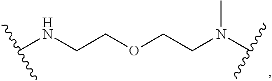 [SMILES: *NCCOCCN(*)C] | (21) |
| 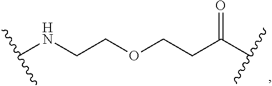 [SMILES: *NCCOCCC(*)=O] | (22) |
| 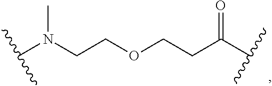 [SMILES: *C(=O)CCOCCN(*)C] | (23) |
| 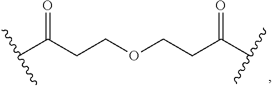 [SMILES: *C(=O)CCOCCC(*)=O] | (24) |
| 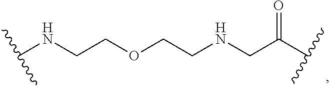 [SMILES: *NCCOCCNCC(*)=O] | (25) |
| 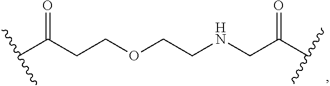 [SMILES: *C(=O)CCOCCNCC(*)=O] | (26) |
| 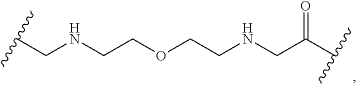 [SMILES: *CNCCOCCNCC(*)=O] | (27) |
| 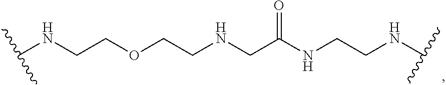 [SMILES: *NCCNC(=O)CNCCOCCN*] | (28) |
| 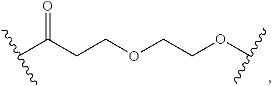 [SMILES: *OCCOCCC(*)=O] | (29) |
| 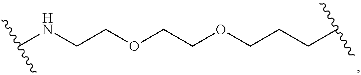 [SMILES: *CCCOCCOCCN*] | (30) |
| 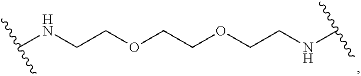 [SMILES: *NCCOCCOCCN*] | (31) |
| 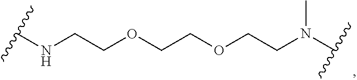 [SMILES: *NCCOCCOCCN(*)C] | (32) |
| 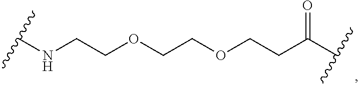 [SMILES: *NCCOCCOCCC(*)=O] | (33) |
| 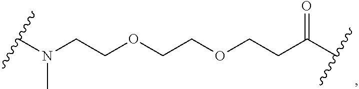 [SMILES: *C(=O)CCOCCOCCN(*)C] | (34) |
| 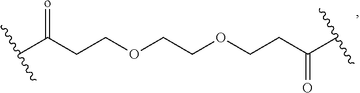 | (35) |
| 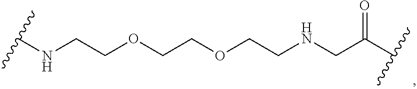 [SMILES: *NCCOCCOCCNCC(*)=O] | (36) |
| 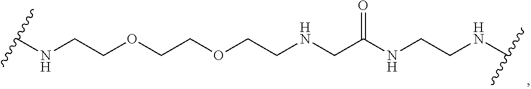 [SMILES: *NCCNC(=O)CNCCOCCOCCN*] | (37) |
| 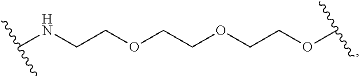 [SMILES: *NCCOCCOCCO*] | (38) |
| 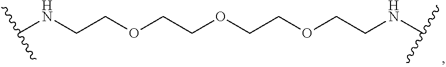 [SMILES: *NCCOCCOCCOCCN*] | (39) |
| 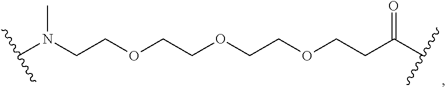 [SMILES: *C(=O)CCOCCOCCOCCN(*)C] | (40) |
| 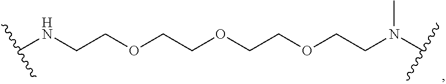 [SMILES: *NCCOCCOCCOCCN(*)C] | (41) |
| 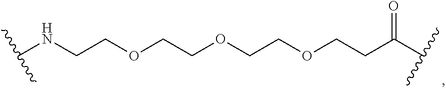 [SMILES: *NCCOCCOCCOCCC(*)=O] | (42) |
| 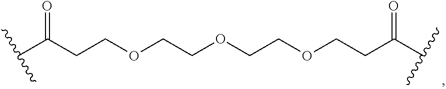 [SMILES: *C(=O)CCOCCOCCOCCC(*)=O] | (43) |
| 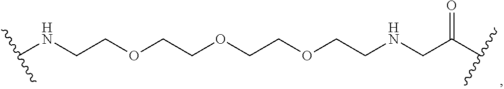 [SMILES: *NCCOCCOCCOCCNCC(*)=O] | (44) |
| 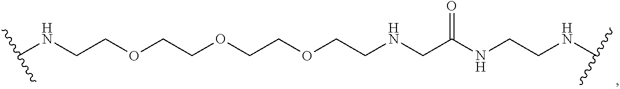 [SMILES: *NCCNC(=O)CNCCOCCOCCOCCN*] | (45) |
| 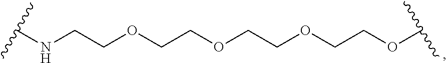 [SMILES: *NCCOCCOCCOCCO*] | (46) |
| 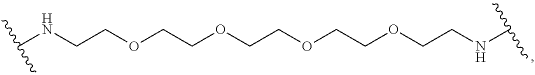 [SMILES: *NCCOCCOCCOCCOCCN*] | (47) |
| 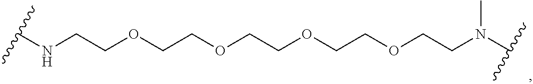 [SMILES: *NCCOCCOCCOCCOCCN(*)C] | (48) |
| 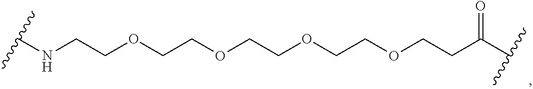 [SMILES: *NCCOCCOCCOCCOCCC(*)=O] | (49) |
| 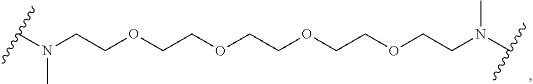 [SMILES: *N(C)CCOCCOCCOCCOCCN(*)C] | (50) |
| 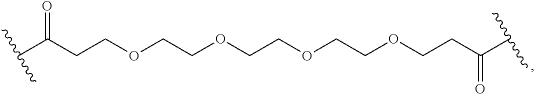 [SMILES: *C(=O)CCOCCOCCOCCOCCC(*)=O] | (51) |
| 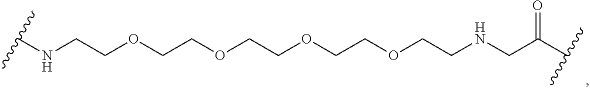 [SMILES: *NCCOCCOCCOCCOCCNCC(*)=O] | (52) |
| 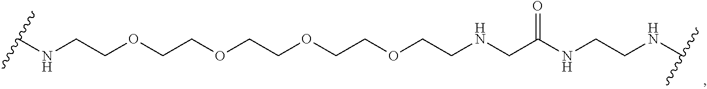 [SMILES: *NCCNC(=O)CNCCOCCOCCOCCOCCN*] | (53) |
| 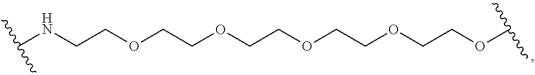 [SMILES: *NCCOCCOCCOCCOCCO*] | (54) |
| 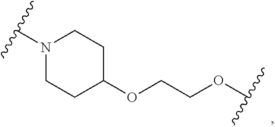 [SMILES: *OCCOC1CCN(*)CC1] | (55) |
| 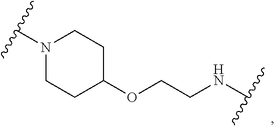 [SMILES: *NCCOC1CCN(*)CC1] | (56) |
| 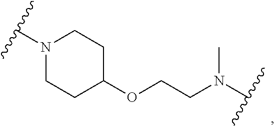 [SMILES: *N(C)CCOC1CCN(*)CC1] | (57) |
| 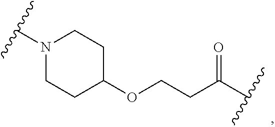 [SMILES: *C(=O)CCOC1CCN(*)CC1] | (58) |
| 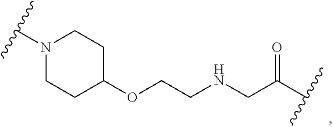 [SMILES: *C(=O)CNCCOC1CCN(*)CC1] | (59) |
| 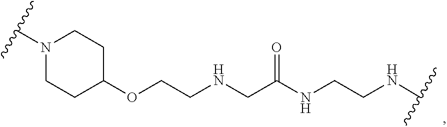 [SMILES: *NCCNC(=O)CNCCOC1CCN(*)CC1] | (60) |
| 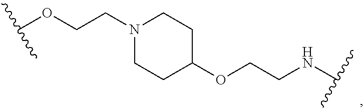 [SMILES: *NCCOC1CCN(CCO*)CC1] | (61) |
| 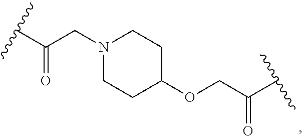 [SMILES: *C(=O)COC1CCN(CC(*)=O)CC1] | (62) |
| 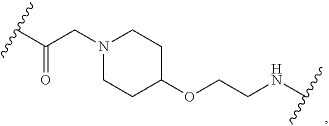 [SMILES: *NCCOC1CCN(CC(*)=O)CC1] | (63) |
| 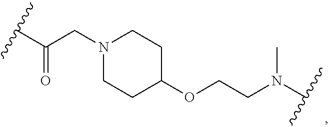 [SMILES: *C(=O)CN1CCC(OCCN(*)C)CC1] | (64) |
| 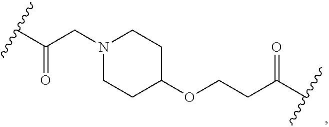 [SMILES: *C(=O)CCOC1CCN(CC(*)=O)CC1] | (65) |
| 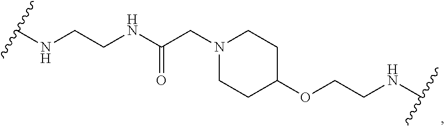 [SMILES: *NCCNC(=O)CN1CCC(OCCN*)CC1] | (66) |
| 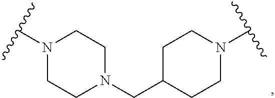 [SMILES: *N1CCC(CN2CCN(*)CC2)CC1] | (67) |
| 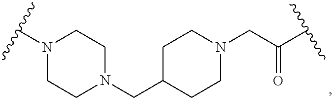 [SMILES: *C(=O)CN1CCC(CN2CCN(*)CC2)CC1] | (68) |
| 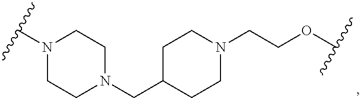 [SMILES: *OCCN1CCC(CN2CCN(*)CC2)CC1] | (69) |
| 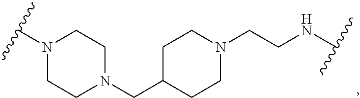 [SMILES: *NCCN1CCC(CN2CCN(*)CC2)CC1] | (70) |
| 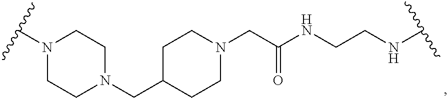 [SMILES: *NCCNC(=O)CN1CCC(CN2CCN(*)CC2)CC1] | (71) |
| 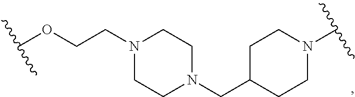 [SMILES: *OCCN1CCN(CC2CCN(*)CC2)CC1] | (72) |
| 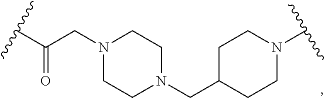 [SMILES: *C(=O)CN1CCN(CC2CCN(*)CC2)CC1] | (73) |
| 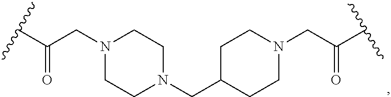 [SMILES: *C(=O)CN1CCC(CN2CCN(CC(*)=O)CC2)CC1] | (74) |
| 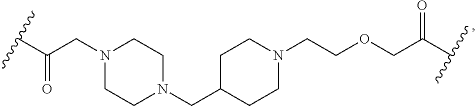 [SMILES: *C(=O)COCCN1CCC(CN2CCN(CC(*)=O)CC2)CC1] | (75) |
| 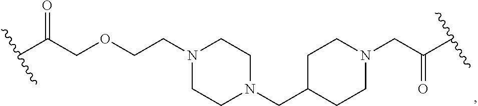 [SMILES: *C(=O)COCCN1CCN(CC2CCN(CC(*)=O)CC2)CC1] | (76) |
| 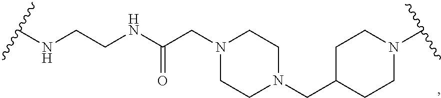 [SMILES: *NCCNC(=O)CN1CCN(CC2CCN(*)CC2)CC1] | (77) |
| 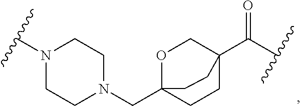 [SMILES: *C(=O)C12CCC(CN3CCN(*)CC3)(CC1)OC2] | (78) |
| 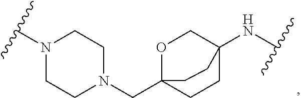 [SMILES: *NC12CCC(CN3CCN(*)CC3)(CC1)OC2] | (79) |
| 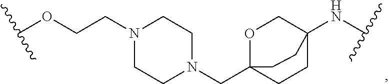 [SMILES: *NC12CCC(CN3CCN(CCO*)CC3)(CC1)OC2] | (80) |
| 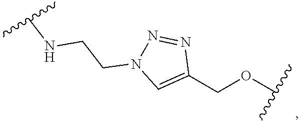 [SMILES: *NCCn1cc(CO*)nn1] | (81) |
| 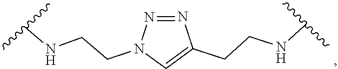 [SMILES: *NCCc1cn(CCN*)nn1] | (82) |
| 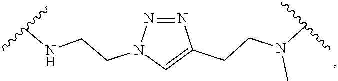 [SMILES: *NCCn1cc(CCN(*)C)nn1] | (83) |
| 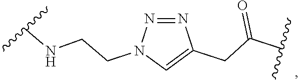 [SMILES: *NCCn1cc(CC(*)=O)nn1] | (84) |
| 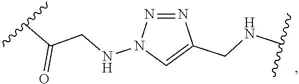 [SMILES: *NCc1cn(NCC(*)=O)nn1] | (85) |
| 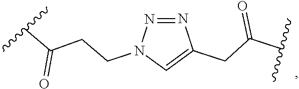 [SMILES: *C(=O)CCn1cc(CC(*)=O)nn1] | (86) |
| 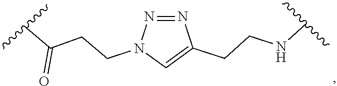 [SMILES: *NCCc1cn(CCC(*)=O)nn1] | (87) |
| 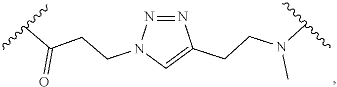 [SMILES: *C(=O)CCn1cc(CCN(*)C)nn1] | (88) |
| 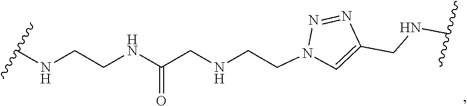 [SMILES: *NCCNC(=O)CNCCn1cc(CN*)nn1] | (89) |
| 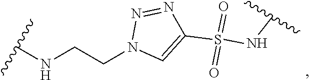 [SMILES: *NCCn1cc(S(=O)(=O)N*)nn1] | (90) |
| 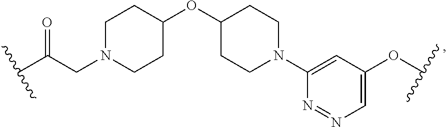 [SMILES: *Oc1cnnc(N2CCC(OC3CCN(CC(*)=O)CC3)CC2)c1] | (91) |
| 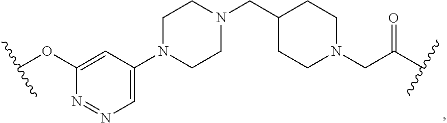 [SMILES: *Oc1cc(N2CCN(CC3CCN(CC(*)=O)CC3)CC2)cnn1] | (92) |
| 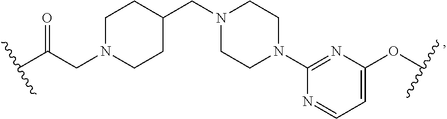 [SMILES: *Oc1ccnc(N2CCN(CC3CCN(CC(*)=O)CC3)CC2)n1] | (93) |
| 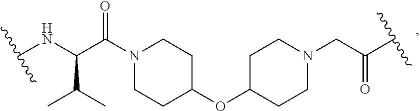 [SMILES: *N[C@@H](C(=O)N1CCC(OC2CCN(CC(*)=O)CC2)CC1)C(C)C] | (94) |
| 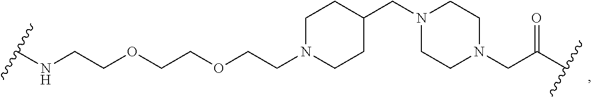 [SMILES: *NCCOCCOCCN1CCC(CN2CCN(CC(*)=O)CC2)CC1] | (95) |
| 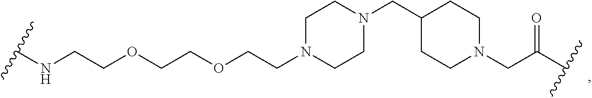 [SMILES: *NCCOCCOCCN1CCN(CC2CCN(CC(*)=O)CC2)CC1] | (96) |
| 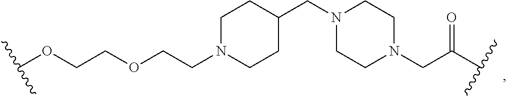 [SMILES: *OCCOCCN1CCC(CN2CCN(CC(*)=O)CC2)CC1] | (97) |
| 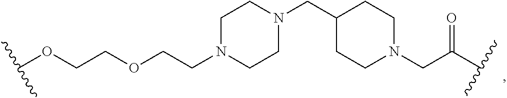 [SMILES: *OCCOCCN1CCN(CC2CCN(CC(*)=O)CC2)CC1] | (98) |
| 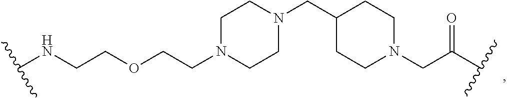 [SMILES: *NCCOCCN1CCN(CC2CCN(CC(*)=O)CC2)CC1] | (99) |
| 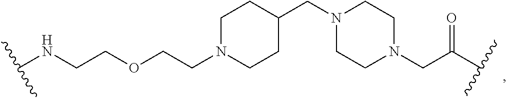 [SMILES: *NCCOCCN1CCC(CN2CCN(CC(*)=O)CC2)CC1] | (100) |
| 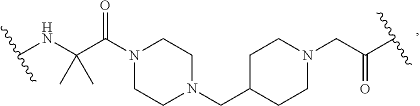 [SMILES: *NC(C)(C)C(=O)N1CCN(CC2CCN(CC(*)=O)CC2)CC1] | (101) |
| 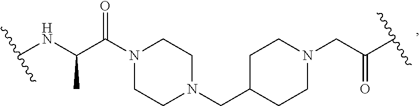 [SMILES: *N[C@H](C)C(=O)N1CCN(CC2CCN(CC(*)=O)CC2)CC1] | (102) |
| 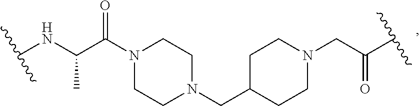 [SMILES: *N[C@@H](C)C(=O)N1CCN(CC2CCN(CC(*)=O)CC2)CC1] | (103) |
| 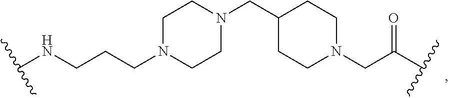 [SMILES: *NCCCN1CCN(CC2CCN(CC(*)=O)CC2)CC1] | (104) |
| 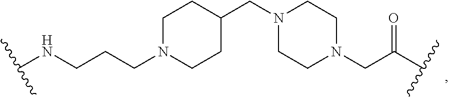 [SMILES: *NCCCN1CCC(CN2CCN(CC(*)=O)CC2)CC1] | (105) |
| 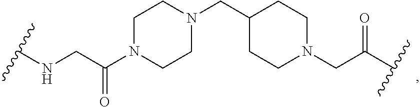 [SMILES: *NCC(=O)N1CCN(CC2CCN(CC(*)=O)CC2)CC1] | (106) |
| 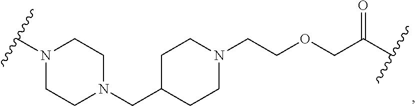 [SMILES: *C(=O)COCCN1CCC(CN2CCN(*)CC2)CC1] | (107) |
| 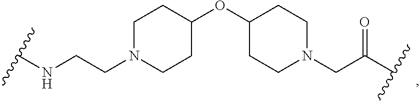 [SMILES: *NCCN1CCC(OC2CCN(CC(*)=O)CC2)CC1] | (108) |
| 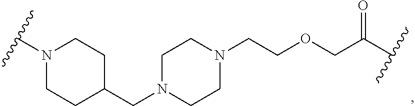 [SMILES: *C(=O)COCCN1CCN(CC2CCN(*)CC2)CC1] | (109) |
| 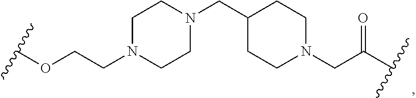 [SMILES: *OCCN1CCN(CC2CCN(CC(*)=O)CC2)CC1] | (110) |
| 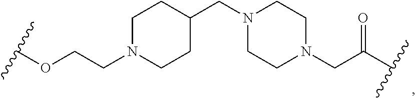 [SMILES: *OCCN1CCC(CN2CCN(CC(*)=O)CC2)CC1] | (111) |
| 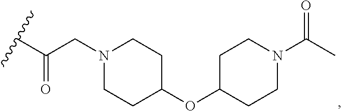 [SMILES: *C(=O)CN1CCC(OC2CCN(C(C)=O)CC2)CC1] | (112) |
| 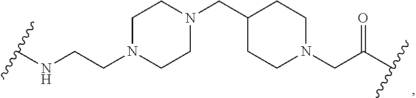 [SMILES: *NCCN1CCN(CC2CCN(CC(*)=O)CC2)CC1] | (113) |
| 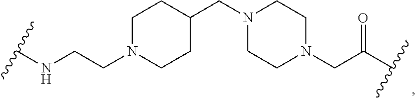 [SMILES: *NCCN1CCC(CN2CCN(CC(*)=O)CC2)CC1] | (114) |
| 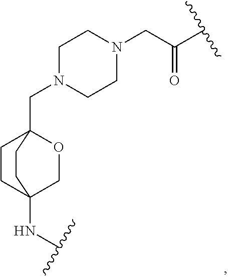 [SMILES: *NC12CCC(CN3CCN(CC(*)=O)CC3)(CC1)OC2] | (115) |
| 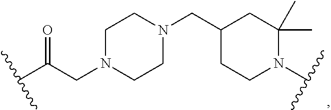 [SMILES: *C(=O)CN1CCN(CC2CCN(*)C(C)(C)C2)CC1] | (116) |
| 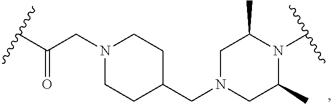 [SMILES: *C(=O)CN1CCC(CN2C[C@@H](C)N(*)[C@@H](C)C2)CC1] | (117) |
| 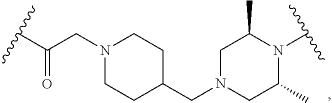 [SMILES: *C(=O)CN1CCC(CN2C[C@@H](C)N(*)[C@H](C)C2)CC1] | (118) |
| 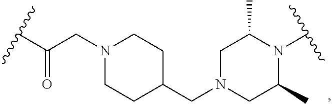 [SMILES: *C(=O)CN1CCC(CN2C[C@H](C)N(*)[C@@H](C)C2)CC1] | (119) |
| 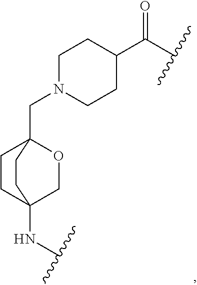 [SMILES: *NC12CCC(CN3CCC(C(*)=O)CC3)(CC1)OC2] | (120) |
| 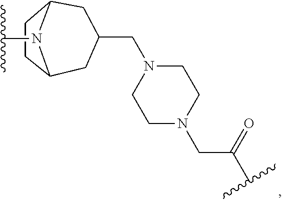 [SMILES: *C(=O)CN1CCN(CC2CC3CCC(C2)N3*)CC1] | (121) |
| 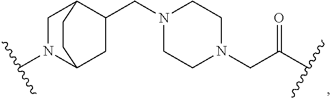 [SMILES: *C(=O)CN1CCN(CC2CC3CCC2CN3*)CC1] | (122) |
| 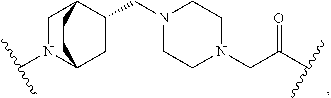 [SMILES: *C(=O)CN1CCN(C[C@@H]2C[C@H]3CC[C@@H]2CN3*)CC1] | (123) |
| 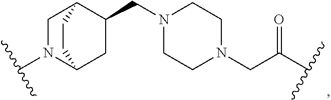 [SMILES: *C(=O)CN1CCN(C[C@H]2C[C@@H]3CC[C@H]2CN3*)CC1] | (124) |
| 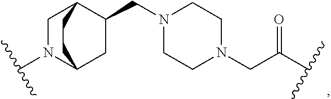 [SMILES: *C(=O)CN1CCN(C[C@H]2C[C@H]3CC[C@@H]2CN3*)CC1] | (125) |
| 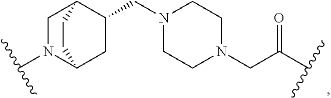 [SMILES: *C(=O)CN1CCN(C[C@@H]2C[C@@H]3CC[C@H]2CN3*)CC1] | (126) |
| 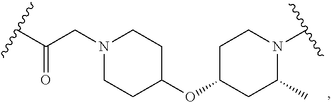 [SMILES: *C(=O)CN1CCC(O[C@@H]2CCN(*)[C@H](C)C2)CC1] | (127) |
| 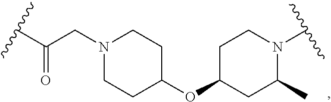 [SMILES: *C(=O)CN1CCC(O[C@H]2CCN(*)[C@@H](C)C2)CC1] | (128) |
| 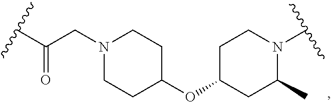 [SMILES: *C(=O)CN1CCC(O[C@@H]2CCN(*)[C@@H](C)C2)CC1] | (129) |
| 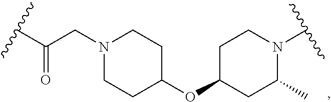 [SMILES: *C(=O)CN1CCC(O[C@H]2CCN(*)[C@H](C)C2)CC1] | (130) |
| 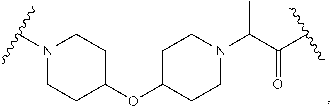 [SMILES: *C(=O)C(C)N1CCC(OC2CCN(*)CC2)CC1] | (131) |
| 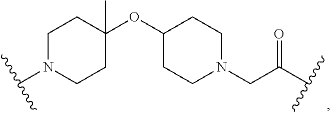 [SMILES: *C(=O)CN1CCC(OC2(C)CCN(*)CC2)CC1] | (132) |
| 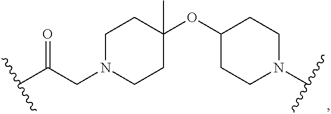 [SMILES: *C(=O)CN1CCC(C)(OC2CCN(*)CC2)CC1] | (133) |
| 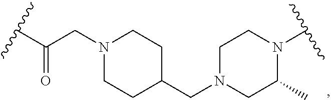 [SMILES: *C(=O)CN1CCC(CN2CCN(*)[C@H](C)C2)CC1] | (134) |
| 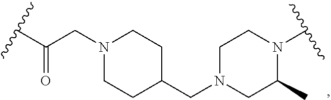 [SMILES: *C(=O)CN1CCC(CN2CCN(*)[C@@H](C)C2)CC1] | (135) |
| 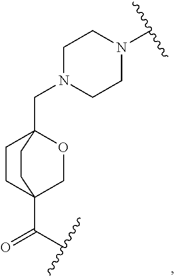 [SMILES: *C(=O)C12CCC(CN3CCN(*)CC3)(CC1)OC2] | (136) |
| 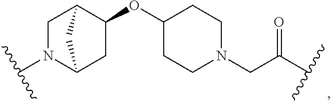 [SMILES: *C(=O)CN1CCC(O[C@H]2C[C@@H]3C[C@H]2CN3*)CC1] | (137) |
| 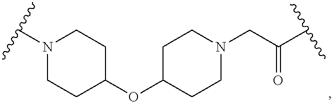 [SMILES: *C(=O)CN1CCC(OC2CCN(*)CC2)CC1] | (138) |
| 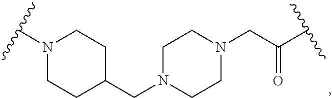 [SMILES: *C(=O)CN1CCN(CC2CCN(*)CC2)CC1] | (139) |
| 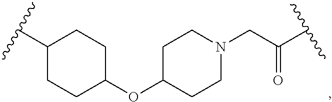 [SMILES: *C(=O)CN1CCC(OC2CCC(*)CC2)CC1] | (140) |
| 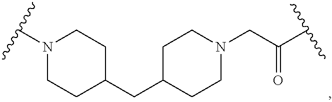 [SMILES: *C(=O)CN1CCC(CC2CCN(*)CC2)CC1] | (141) |
| 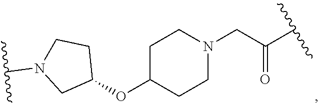 [SMILES: *C(=O)CN1CCC(O[C@H]2CCN(*)C2)CC1] | (142) |
| 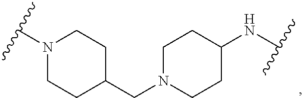 [SMILES: *NC1CCN(CC2CCN(*)CC2)CC1] | (143) |
| 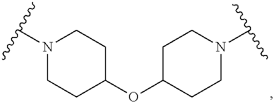 [SMILES: *N1CCC(OC2CCN(*)CC2)CC1] | (144) |
| 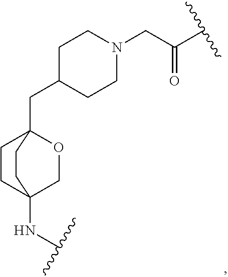 [SMILES: *NC12CCC(CC3CCN(CC(*)=O)CC3)(CC1)OC2] | (145) |
| 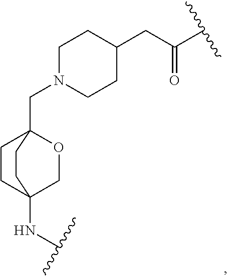 [SMILES: *NC12CCC(CN3CCC(CC(*)=O)CC3)(CC1)OC2] | (146) |
| 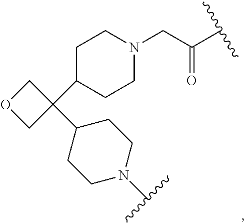 [SMILES: *C(=O)CN1CCC(C2(C3CCN(*)CC3)COC2)CC1] | (147) |
| 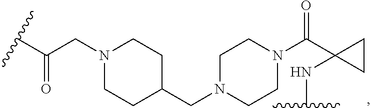 [SMILES: *NC1(C(=O)N2CCN(CC3CCN(CC(*)=O)CC3)CC2)CC1] | (148) |
| 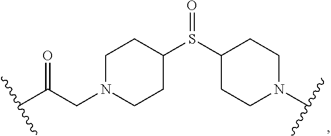 [SMILES: *C(=O)CN1CCC(S(=O)C2CCN(*)CC2)CC1] | (149) |
| 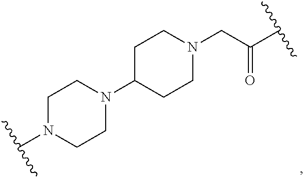 [SMILES: *C(=O)CN1CCC(N2CCN(*)CC2)CC1] | (150) |
| 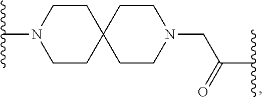 [SMILES: *C(=O)CN1CCC2(CCN(*)CC2)CC1] | (151) |
| 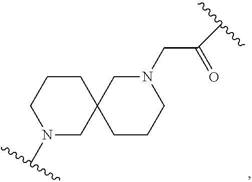 [SMILES: *C(=O)CN1CCCC2(CCCN(*)C2)C1] | (152) |
| 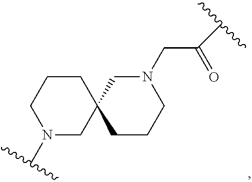 [SMILES: *C(=O)CN1CCC[C@@]2(CCCN(*)C2)C1] | (153) |
| 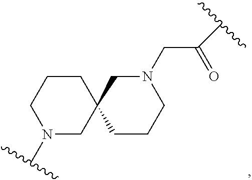 [SMILES: *C(=O)CN1CCC[C@]2(CCCN(*)C2)C1] | (154) |
| 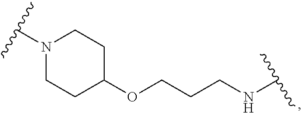 [SMILES: *NCCCOC1CCN(*)CC1] | (155) |
| 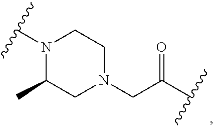 [SMILES: *C(=O)CN1CCN(*)[C@H](C)C1] | (156) |
| 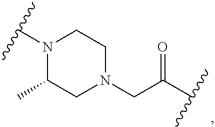 [SMILES: *C(=O)CN1CCN(*)[C@@H](C)C1] | (157) |
| 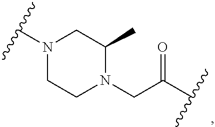 [SMILES: *C(=O)CN1CCN(*)C[C@H]1C] | (158) |
| 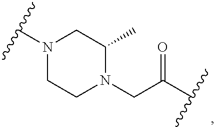 [SMILES: *C(=O)CN1CCN(*)C[C@@H]1C] | (159) |
| 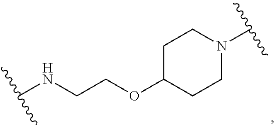 [SMILES: *NCCOC1CCN(*)CC1] | (160) |
| 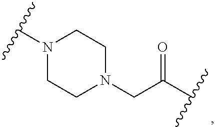 [SMILES: *C(=O)CN1CCN(*)CC1] | (161) |
| 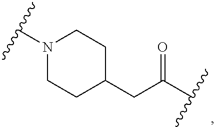 [SMILES: *C(=O)CC1CCN(*)CC1] | (162) |
| 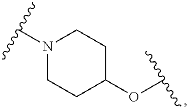 [SMILES: *OC1CCN(*)CC1] | (163) |
| 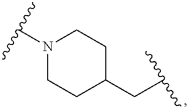 [SMILES: *CC1CCN(*)CC1] | (164) |
| 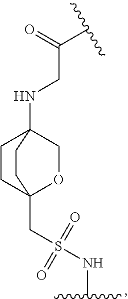 [SMILES: *NS(=O)(=O)CC12CCC(NCC(*)=O)(CC1)CO2] | (165) |
| 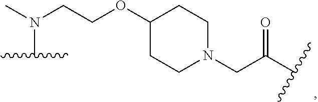 [SMILES: *C(=O)CN1CCC(OCCN(*)C)CC1] | (166) |
| 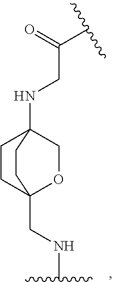 [SMILES: *NCC12CCC(NCC(*)=O)(CC1)CO2] | (167) |
| 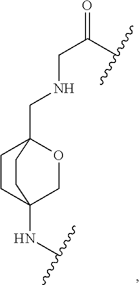 [SMILES: *NC12CCC(CNCC(*)=O)(CC1)OC2] | (168) |
| 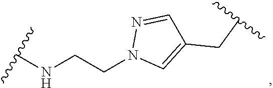 [SMILES: *Cc1cnn(CCN*)c1] | (169) |
| 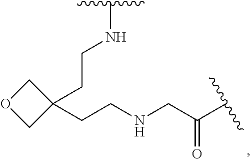 [SMILES: *NCCC1(CCNCC(*)=O)COC1] | (170) |
| 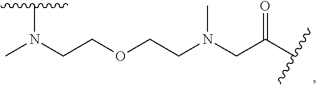 [SMILES: *C(=O)CN(C)CCOCCN(*)C] | (171) |
| 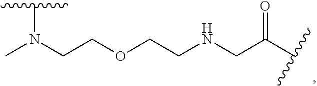 [SMILES: *C(=O)CNCCOCCN(*)C] | (172) |
| 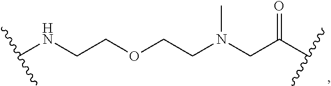 [SMILES: *NCCOCCN(C)CC(*)=O] | (173) |
| 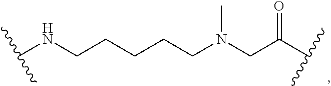 [SMILES: *NCCCCCN(C)CC(*)=O] | (174) |
| 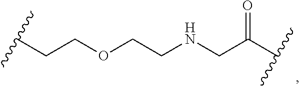 [SMILES: *CCOCCNCC(*)=O] | (175) |
| 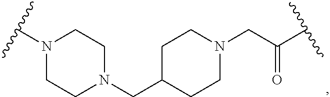 [SMILES: *C(=O)CN1CCC(CN2CCN(*)CC2)CC1] | (176) |
| 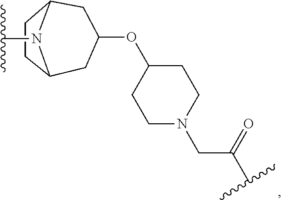 [SMILES: *C(=O)CN1CCC(OC2CC3CCC(C2)N3*)CC1] | (177) |
| 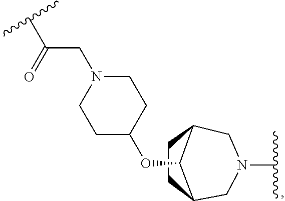 [SMILES: *C(=O)CN1CCC(O[C@H]2[C@@H]3CC[C@H]2CN(*)C3)CC1] | (178) |
| 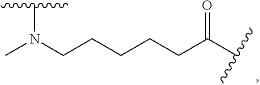 [SMILES: *C(=O)CCCCCN(*)C] | (179) |
| 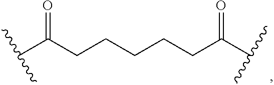 [SMILES: *C(=O)CCCCCC(*)=O] | (180) |
| 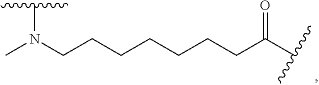 [SMILES: *C(=O)CCCCCCCN(*)C] | (181) |
| 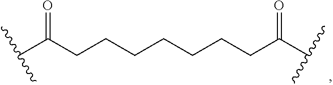 [SMILES: *C(=O)CCCCCCCC(*)=O] | (182) |
| 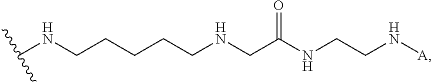 | (183) |
| 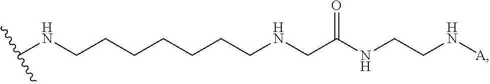 | (184) |
| 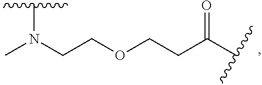 [SMILES: *C(=O)CCOCCN(*)C] | (185) |
| 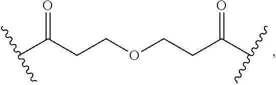 [SMILES: *C(=O)CCOCCC(*)=O] | (186) |
| 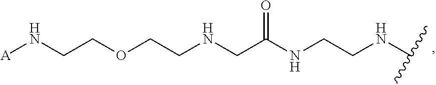 | (187) |
| 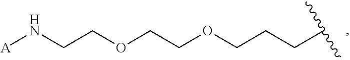 | (188) |
| 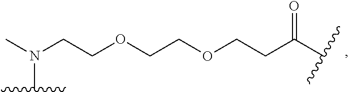 [SMILES: *C(=O)CCOCCOCCN(*)C] | (189) |
| 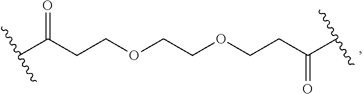 [SMILES: *C(=O)CCOCCOCCC(*)=O] | (190) |
| 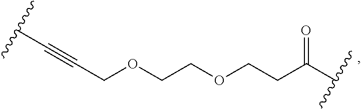 [SMILES: *C#CCOCCOCCC(*)=O] | (191) |
| 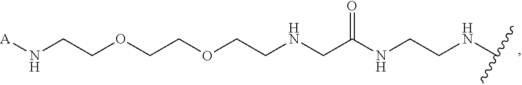 | (192) |
| 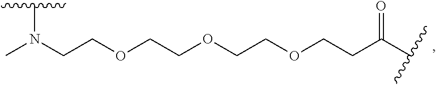 [SMILES: *C(=O)CCOCCOCCOCCN(*)C] | (193) |
| 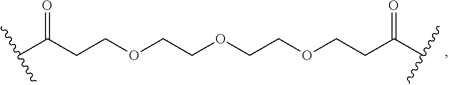 [SMILES: *C(=O)CCOCCOCCOCCC(*)=O] | (194) |
| 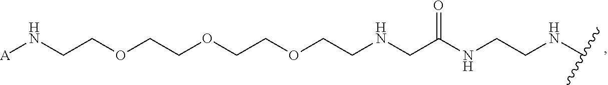 | (195) |
| 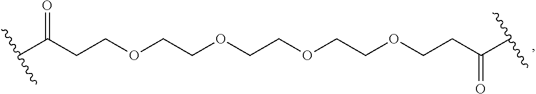 [SMILES: *C(=O)CCOCCOCCOCCOCCC(*)=O] | (196) |
| 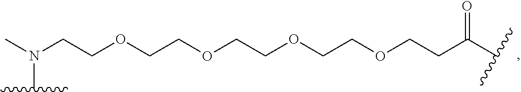 [SMILES: *C(=O)CCOCCOCCOCCOCCN(*)C] | (197) |
| 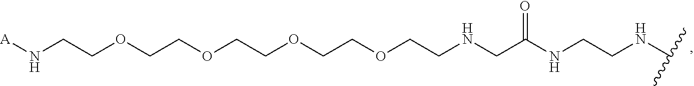 | (198) |
| 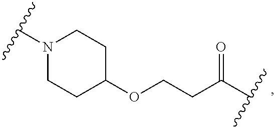 [SMILES: *C(=O)CCOC1CCN(*)CC1] | (199) |
| 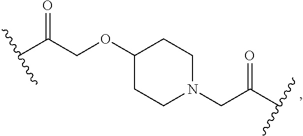 [SMILES: *C(=O)COC1CCN(CC(*)=O)CC1] | (200) |
| 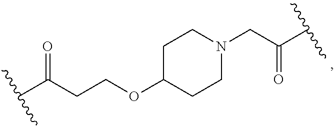 [SMILES: *C(=O)CCOC1CCN(CC(*)=O)CC1] | (201) |
| 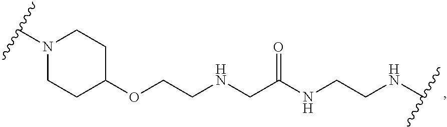 [SMILES: *NCCNC(=O)CNCCOC1CCN(*)CC1] | (202) |
| 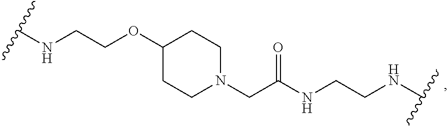 [SMILES: *NCCNC(=O)CN1CCC(OCCN*)CC1] | (203) |
| 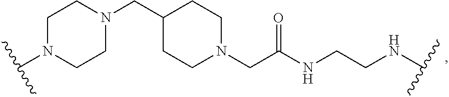 [SMILES: *NCCNC(=O)CN1CCC(CN2CCN(*)CC2)CC1] | (204) |
| 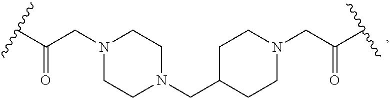 [SMILES: *C(=O)CN1CCC(CN2CCN(CC(*)=O)CC2)CC1] | (205) |
| 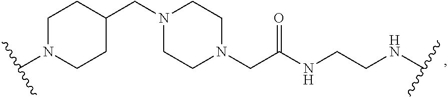 [SMILES: *NCCNC(=O)CN1CCN(CC2CCN(*)CC2)CC1] | (206) |
| 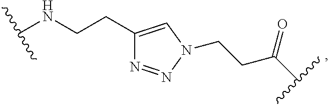 [SMILES: *NCCc1cn(CCC(*)=O)nn1] | (207) |
| 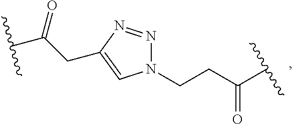 [SMILES: *C(=O)CCn1cc(CC(*)=O)nn1] | (208) |
| 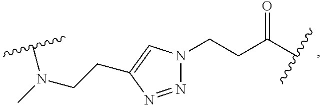 [SMILES: *C(=O)CCn1cc(CCN(*)C)nn1] | (209) |
| 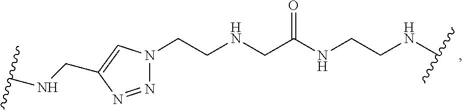 [SMILES: *NCCNC(=O)CNCCn1cc(CN*)nn1] | (210) |
| 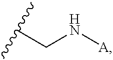 | (211) |
| 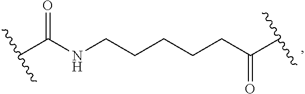 [SMILES: *C(=O)CCCCCNC(*)=O] | (212) |
| 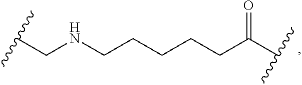 [SMILES: *CNCCCCCC(*)=O] | (213) |
| 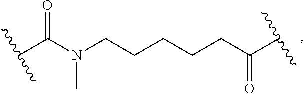 [SMILES: *C(=O)CCCCCN(C)C(*)=O] | (214) |
| 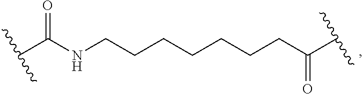 [SMILES: *C(=O)CCCCCCCNC(*)=O] | (215) |
| 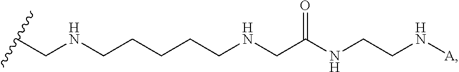 | (216) |
| 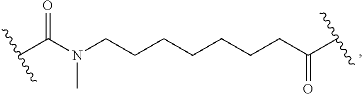 [SMILES: *C(=O)CCCCCCCN(C)C(*)=O] | (217) |
| 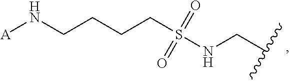 | (218) |
| 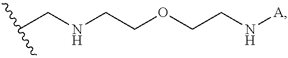 | (219) |
| 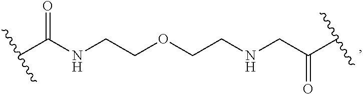 [SMILES: *C(=O)CNCCOCCNC(*)=O] | (220) |
| 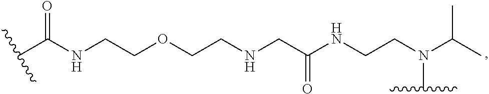 [SMILES: *C(=O)NCCOCCNCC(=O)NCCN(*)C(C)C] | (221) |
| 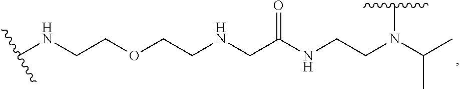 [SMILES: *NCCOCCNCC(=O)NCCN(*)C(C)C] | (222) |
| 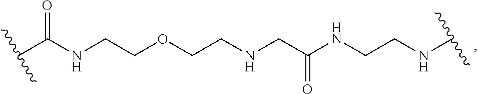 [SMILES: *NCCNC(=O)CNCCOCCNC(*)=O] | (223) |
| 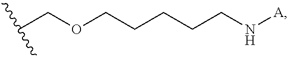 | (224) |
| 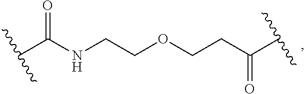 [SMILES: *C(=O)CCOCCNC(*)=O] | (225) |
| 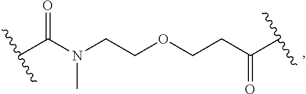 [SMILES: *C(=O)CCOCCN(C)C(*)=O] | (226) |
| 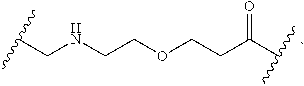 [SMILES: *CNCCOCCC(*)=O] | (227) |
| 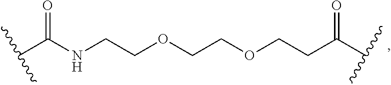 [SMILES: *C(=O)CCOCCOCCNC(*)=O] | (228) |
| 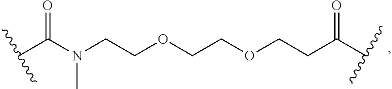 [SMILES: *C(=O)CCOCCOCCN(C)C(*)=O] | (229) |
| 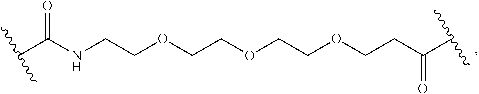 [SMILES: *C(=O)CCOCCOCCOCCNC(*)=O] | (230) |
| 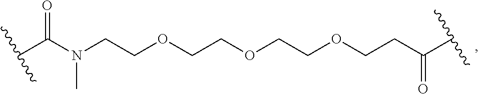 [SMILES: *C(=O)CCOCCOCCOCCN(C)C(*)=O] | (231) |
| 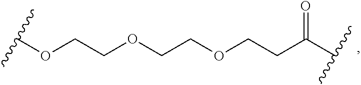 [SMILES: *OCCOCCOCCC(*)=O] | (232) |
| 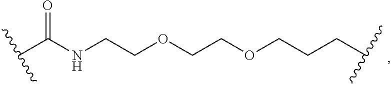 [SMILES: *CCCOCCOCCNC(*)=O] | (233) |
| 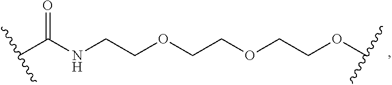 [SMILES: *OCCOCCOCCNC(*)=O] | (234) |
| 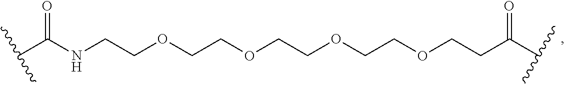 [SMILES: *C(=O)CCOCCOCCOCCOCCNC(*)=O] | (235) |
| 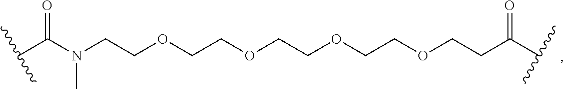 [SMILES: *C(=O)CCOCCOCCOCCOCCN(C)C(*)=O] | (236) |
| 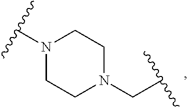 [SMILES: *CN1CCN(*)CC1] | (237) |
| 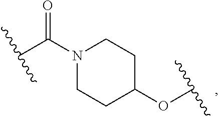 [SMILES: *OC1CCN(C(*)=O)CC1] | (238) |
| 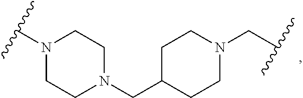 [SMILES: *CN1CCC(CN2CCN(*)CC2)CC1] | (239) |
| 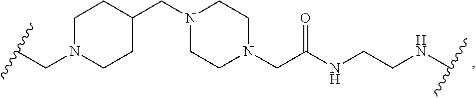 [SMILES: *CN1CCC(CN2CCN(CC(=O)NCCN*)CC2)CC1] | (240) |
| 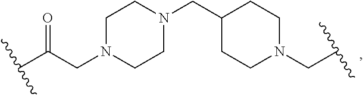 [SMILES: *CN1CCC(CN2CCN(CC(*)=O)CC2)CC1] | (241) |
| 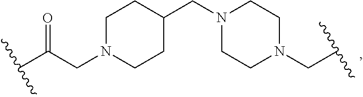 [SMILES: *CN1CCN(CC2CCN(CC(*)=O)CC2)CC1] | (242) |
| 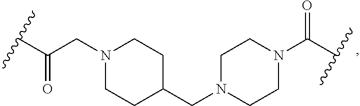 [SMILES: *C(=O)CN1CCC(CN2CCN(C(*)=O)CC2)CC1] | (243) |
| 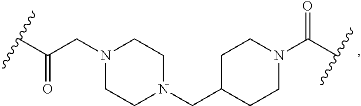 [SMILES: *C(=O)CN1CCN(CC2CCN(C(*)=O)CC2)CC1] | (244) |
| 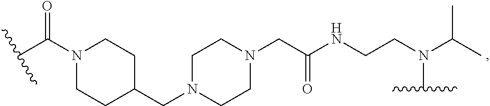 [SMILES: *C(=O)N1CCC(CN2CCN(CC(=O)NCCN(*)C(C)C)CC2)CC1] | (245) |
| 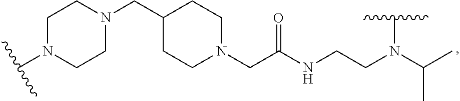 [SMILES: *N1CCN(CC2CCN(CC(=O)NCCN(*)C(C)C)CC2)CC1] | (246) |
| 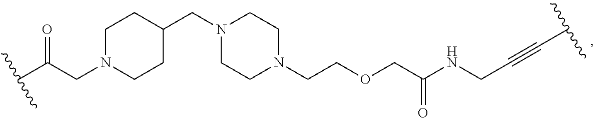 [SMILES: *C#CCNC(=O)COCCN1CCN(CC2CCN(CC(*)=O)CC2)CC1] | (247) |
| 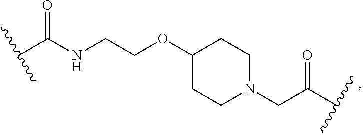 [SMILES: *C(=O)CN1CCC(OCCNC(*)=O)CC1] | (248) |
| 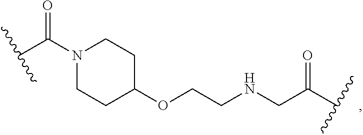 [SMILES: *C(=O)CNCCOC1CCN(C(*)=O)CC1] | (249) |
| 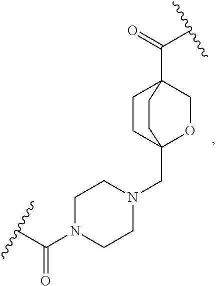 [SMILES: *C(=O)N1CCN(CC23CCC(C(*)=O)(CC2)CO3)CC1] | (250) |
| 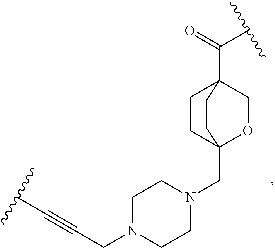 [SMILES: *C#CCN1CCN(CC23CCC(C(*)=O)(CC2)CO3)CC1] | (251) |
| 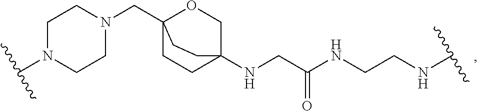 [SMILES: *NCCNC(=O)CNC12CCC(CN3CCN(*)CC3)(CC1)OC2] | (252) |
| 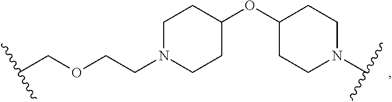 [SMILES: *COCCN1CCC(OC2CCN(*)CC2)CC1] | (253) |
| 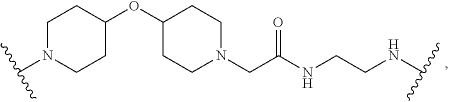 [SMILES: *NCCNC(=O)CN1CCC(OC2CCN(*)CC2)CC1] | (254) |
| 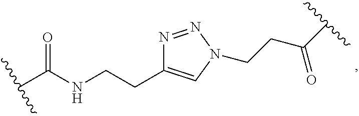 | (255) |
| 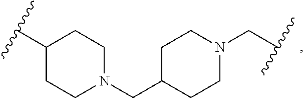 [SMILES: *CN1CCC(CN2CCC(*)CC2)CC1] | (256) |
| 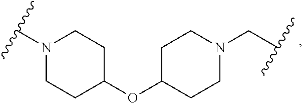 [SMILES: *CN1CCC(OC2CCN(*)CC2)CC1] | (257) |
| 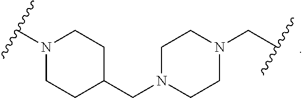 [SMILES: *CN1CCN(CC2CCN(*)CC2)CC1] | (258) |

In some embodiments, the present invention provides a compound having PBM described and disclosed herein, DBM set forth in Table A above, and a linker set forth in Table B above, or a pharmaceutically acceptable salt thereof.

Exemplary compounds of the invention are set forth in Table 1, below.

Lengthy table referenced here
US20250127907A1-20250424-T00001
Please refer to the end of the specification for access instructions.

In some embodiments, the present invention provides a compound set forth in Table 1, above, or a pharmaceutically acceptable salt thereof.

### General Methods of Providing the Present Compounds

The compounds of this invention may be prepared or isolated in general by synthetic and/or semi-synthetic methods known to those skilled in the art for analogous compounds and by methods described in detail in the Examples, herein.

In the Schemes below, where a particular protecting group, leaving group, or transformation condition is depicted, one of ordinary skill in the art will appreciate that other protecting groups, leaving groups, and transformation conditions are also suitable and are contemplated. Such groups and transformations are described in detail in March's Advanced Organic Chemistry: Reactions, Mechanisms, and Structure, M. B. Smith and J. March, 5^{th} Edition, John Wiley & Sons, 2001, Comprehensive Organic Transformations, R. C. Larock, 2^{nd} Edition, John Wiley & Sons, 1999, and Protecting Groups in Organic Synthesis, T. W. Greene and P. G. M. Wuts, 3^{rd} edition, John Wiley & Sons, 1999, the entirety of each of which is hereby incorporated herein by reference.

As used herein, the phrase “oxygen protecting group” includes, for example, carbonyl protecting groups, hydroxyl protecting groups, etc. Hydroxyl protecting groups are well known in the art and include those described in detail in Protecting Groups in Organic Synthesis, T. W. Greene and P. G. M. Wuts, 3^{rd} edition, John Wiley & Sons, 1999, the entirety of each of which is herein incorporated by reference. Examples of suitable hydroxyl protecting groups include, but are not limited to, esters, allyl ethers, ethers, silyl ethers, alkyl ethers, arylalkyl ethers, and alkoxyalkyl ethers. Examples of such esters include formates, acetates, carbonates, and sulfonates. Specific examples include formate, benzoyl formate, chloroacetate, trifluoroacetate, methoxyacetate, triphenylmethoxyacetate, p-chlorophenoxyacetate, 3-phenylpropionate, 4-oxopentanoate, 4,4-(ethylenedithio)pentanoate, pivaloate (trimethylacetyl), crotonate, 4-methoxy-crotonate, benzoate, p-benylbenzoate, 2,4,6-trimethylbenzoate, carbonates such as methyl, 9-fluorenylmethyl, ethyl, 2,2,2-trichloroethyl, 2-(trimethylsilyl)ethyl, 2-(phenylsulfonyl)ethyl, vinyl, allyl, and p-nitrobenzyl. Examples of such silyl ethers include trimethylsilyl, triethylsilyl, t-butyldimethylsilyl, t-butyldiphenylsilyl, triisopropylsilyl, and other trialkylsilyl ethers. Alkyl ethers include methyl, benzyl, p-methoxybenzyl, 3,4-dimethoxybenzyl, trityl, t-butyl, allyl, and allyloxycarbonyl ethers or derivatives. Alkoxyalkyl ethers include acetals such as methoxymethyl, methylthiomethyl, (2-methoxyethoxy)methyl, benzyloxymethyl, beta-(trimethylsilyl)ethoxymethyl, and tetrahydropyranyl ethers. Examples of arylalkyl ethers include benzyl, p-methoxybenzyl (MPM), 3,4-dimethoxybenzyl, O-nitrobenzyl, p-nitrobenzyl, p-halobenzyl, 2,6-dichlorobenzyl, p-cyanobenzyl, and 2- and 4-picolyl.

Amino protecting groups are well known in the art and include those described in detail in Protecting Groups in Organic Synthesis, T. W. Greene and P. G. M. Wuts, 3^{rd} edition, John Wiley & Sons, 1999, the entirety of each of which is herein incorporated by reference. Suitable amino protecting groups include, but are not limited to, aralkylamines, carbamates, cyclic imides, allyl amines, amides, and the like. Examples of such groups include t-butyloxycarbonyl (BOC), ethyloxycarbonyl, methyloxycarbonyl, trichloroethyloxycarbonyl, allyloxycarbonyl (Alloc), benzyloxocarbonyl (CBZ), allyl, phthalimide, benzyl (Bn), fluorenylmethylcarbonyl (Fmoc), formyl, acetyl, chloroacetyl, dichloroacetyl, trichloroacetyl, phenylacetyl, trifluoroacetyl, benzoyl, and the like.

In the schemes below, where a provided compound is formed having a reactive moiety (e.g., amine, alcohol, etc.), it is not shown but it is generally appreciated and well known by those having ordinary skill in the art that the reactivity of said reactive moiety may be masked by employing a suitable protecting group that can thereafter be removed in situ or during a separate synthetic step.

In some embodiments, compounds of the present invention are generally prepared according to Scheme A set forth below:

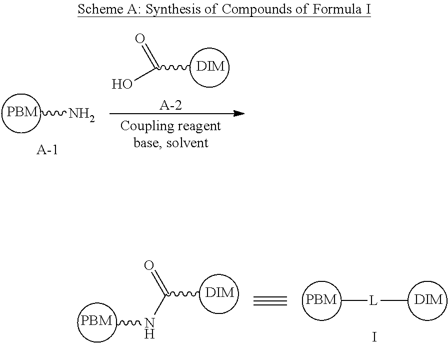

As depicted in Scheme A, above, amine A-1 is coupled to acid A-2 using the a coupling reagent in the presence of the base (e.g., DIPEA) in a solvent (e.g., DMF) to form a compound of formula I with a linker comprising an amide bond. The squiggly bond, “—”, represents the portion of the linker between CBM and the terminal amino group of A-1 or the portion of the linker between DIM and the terminal carboxyl group of A-2, respectively. The amide bond can be formed using coupling reagents known in the art such as, but not limited to DCC, DIC, EDC, HATU, HBTU, HCTU, PyAOP, PyBOP, PyBrOP, BOP, BOP—Cl, DEPBT, T3P, TATU, TBTU, TNTU, TOTU, TPTU, TSTU, or TDBTU.

In some embodiments, compounds of the present invention are generally prepared according to Scheme B set forth below:

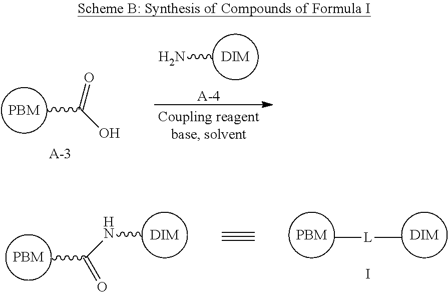

As depicted in Scheme B, above, acid A-3 is coupled to amine A-4 using a coupling reagent in the presence of the base (e.g., DIPEA) in a solvent (e.g., DMF) to form a compound of formula I with a linker comprising an amide bond. The squiggly bond, “—”, represents the portion of the linker between CBM and the terminal carboxyl group of A-3 or the portion of the linker between DIM and the terminal amino group of A-4, respectively. The amide bond can be formed using coupling reagents known in the art such as, but not limited to DCC, DIC, EDC, HATU, HBTU, HCTU, PyAOP, PyBOP, PyBrOP, BOP, BOP—Cl, DEPBT, T3P, TATU, TBTU, TNTU, TOTU, TPTU, TSTU, or TDBTU.

In some embodiments, compounds of the present invention are generally prepared according to Scheme C set forth below:

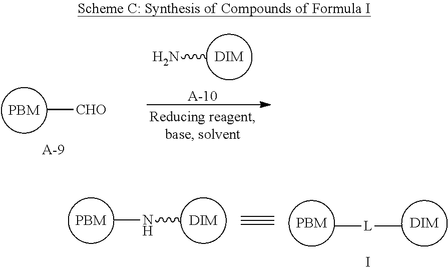

As depicted in Scheme C, above, reductive amination of the mixture of aldehyde A-9 and amine A-10 is effected in the presence of a reducing agent (e.g., NaHB(OAc)₃) and base (e.g., KOAc) in a solvent (e.g., DMF/THF) to form a compound of formula I with a linker comprising a secondary amine. The squiggly bond, “—”, represents the portion of the linker between DIM and the terminal amino group of A-8.

One of skill in the art will appreciate that various functional groups present in compounds of the invention such as aliphatic groups, alcohols, carboxylic acids, esters, amides, aldehydes, halogens and nitriles can be interconverted by techniques well known in the art including, but not limited to reduction, oxidation, esterification, hydrolysis, partial oxidation, partial reduction, halogenation, dehydration, partial hydration, and hydration. “March's Advanced Organic Chemistry”, 5^{th} Ed., Ed.: Smith, M. B. and March, J., John Wiley & Sons, New York: 2001, the entirety of which is incorporated herein by reference. Such interconversions may require one or more of the aforementioned techniques, and certain methods for synthesizing compounds of the invention are described below in the Exemplification.

### Uses, Formulation and Administration

### Pharmaceutically Acceptable Compositions

In some embodiments, the invention provides a composition comprising a compound of this invention or a pharmaceutically acceptable derivative thereof and a pharmaceutically acceptable carrier, adjuvant, or vehicle. In some embodiments, the amount of compound in compositions of this invention is such that it is effective to measurably inhibit a Parp protein, or a mutant thereof, in a biological sample or in a patient. In some embodiments, a composition of this invention is formulated for administration to a patient in need of such composition. In some embodiments, a composition of this invention is formulated for oral administration to a patient.

The term “patient,” as used herein, means an animal, preferably a mammal, and most preferably a human.

The term “pharmaceutically acceptable carrier, adjuvant, or vehicle” refers to a non-toxic carrier, adjuvant, or vehicle that does not destroy the pharmacological activity of the compound with which it is formulated. Pharmaceutically acceptable carriers, adjuvants or vehicles that may be used in the compositions of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

A “pharmaceutically acceptable derivative” means any non-toxic salt, ester, salt of an ester or other derivative of a compound of this invention that, upon administration to a recipient, is capable of providing, either directly or indirectly, a compound of this invention or an inhibitorily active metabolite or residue thereof.

As used herein, the term “inhibitorily active metabolite or residue thereof” means that a metabolite or residue thereof is also an inhibitor of a Parp protein, or a mutant thereof.

Compositions of the present invention may be administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. Preferably, the compositions are administered orally, intraperitoneally or intravenously. Sterile injectable forms of the compositions of this invention may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium.

For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

Pharmaceutically acceptable compositions of this invention may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added.

Alternatively, pharmaceutically acceptable compositions of this invention may be administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax and polyethylene glycols.

Pharmaceutically acceptable compositions of this invention may also be administered topically, especially when the target of treatment includes areas or organs readily accessible by topical application, including diseases of the eye, the skin, or the lower intestinal tract. Suitable topical formulations are readily prepared for each of these areas or organs.

Topical application for the lower intestinal tract can be effected in a rectal suppository formulation (see above) or in a suitable enema formulation. Topically-transdermal patches may also be used.

For topical applications, provided pharmaceutically acceptable compositions may be formulated in a suitable ointment containing the active component suspended or dissolved in one or more carriers. Carriers for topical administration of compounds of this invention include, but are not limited to, mineral oil, liquid petrolatum, white petrolatum, propylene glycol, polyoxyethylene, polyoxypropylene compound, emulsifying wax and water. Alternatively, provided pharmaceutically acceptable compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

For ophthalmic use, provided pharmaceutically acceptable compositions may be formulated as micronized suspensions in isotonic, pH adjusted sterile saline, or, preferably, as solutions in isotonic, pH adjusted sterile saline, either with or without a preservative such as benzylalkonium chloride. Alternatively, for ophthalmic uses, the pharmaceutically acceptable compositions may be formulated in an ointment such as petrolatum.

Pharmaceutically acceptable compositions of this invention may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

Most preferably, pharmaceutically acceptable compositions of this invention are formulated for oral administration. Such formulations may be administered with or without food. In some embodiments, pharmaceutically acceptable compositions of this invention are administered without food. In other embodiments, pharmaceutically acceptable compositions of this invention are administered with food.

The amount of compounds of the present invention that may be combined with the carrier materials to produce a composition in a single dosage form will vary depending upon the host treated, the particular mode of administration. Preferably, provided compositions should be formulated so that a dosage of between 0.01-100 mg/kg body weight/day of the compound can be administered to a patient receiving these compositions.

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of a compound of the present invention in the composition will also depend upon the particular compound in the composition.

### Uses of Compounds and Pharmaceutically Acceptable Compositions

Compounds and compositions described herein are generally useful for the inhibition of kinase activity of one or more enzymes.

As used herein, the terms “PARP1-mediated”, “PARP2-mediated”, “PARP3-mediated”, “PARP4-mediated”, “PARP5a-mediated”, PARP5b-mediated”, “PARP6-mediated”, “PARP7-mediated”, “PARP8-mediated”, “PARP9-mediated”, “PARP10-mediated”, “PARP11-mediated”, “PARP12-mediated”, “PARP13-mediated”, “PARP14-mediated”, “PARP15-mediated”, “PARP16-mediated”, and/or “PARP17-mediated” disorders, diseases, and/or conditions as used herein means any disease or other deleterious condition in which one or more of PARP1, PARP2, PARP3, PARP4, PARP-5a, PARP-5b, PARP6, PARP7, PARP8, PARP9, PARP10, PARP11, PARP12, PARP 13, PARP14, PARP15, PARP16 and/or PARP17 or a mutant thereof, are known to play a role. Accordingly, another embodiment of the present invention relates to treating or lessening the severity of one or more diseases in which one or more of PARP1, PARP2, PARP3, PARP4, PARP-5a, PARP-5b, PARP6, PARP7, PARP8, PARP9, PARP10, PARP11, PARP12, PARP 13, PARP14, PARP15, PARP16 and/or PARP17 or a mutant thereof, are known to play a role.

Compounds of the present disclosure can inhibit PARP and therefore are useful for treating diseases wherein the underlying pathology is, wholly or partially, mediated by PARP. Such diseases include cancer and other diseases with proliferation disorder. In some embodiments, the present disclosure provides treatment of an individual or a patient in vivo using a provided compound or a pharmaceutically acceptable salt thereof such that growth of cancerous tumors is inhibited. A provided compound or a pharmaceutically acceptable salt thereof can be used to inhibit the growth of cancerous tumors with aberrations that activate PARP activity. These include, but not limited to, disease (e.g., cancers) that are characterized by amplification or overexpression of PARP such as ovarian cancer, uterine carcinosarcoma and breast cancer and p27 inactivation such as breast cancer and melanomas. Accordingly, in some embodiments of the methods, the patient has been previously determined to have an amplification of the PARP gene in a biological sample obtained from the human subject that is higher than a control expression level of PARP. Alternatively, a provided compound or a pharmaceutically acceptable salt thereof can be used in conjunction with other agents or standard cancer treatments, as described below. In one embodiment, the present disclosure provides a method for inhibiting growth of tumor cells in vitro. The method includes contacting the tumor cells in vitro with a provided compound or a pharmaceutically acceptable salt thereof. In another embodiment, the present disclosure provides a method for inhibiting growth of tumor cells with PARP amplification and overexpression in an individual or a patient. The method includes administering to the individual or patient in need thereof a therapeutically effective amount of a provided compound or a pharmaceutically acceptable salt thereof.

In some embodiments, provided herein is a method of inhibiting PARP, comprising contacting the PARP with a provided compound or a pharmaceutically acceptable salt thereof. In some embodiments, provided herein is a method of inhibiting PARP in a patient, comprising administering to the patient a provided compound or a pharmaceutically acceptable salt thereof.

In some embodiments, provided herein is a method for treating cancer. The method includes administering to a patient (in need thereof), a therapeutically effective amount of a provided compound or a pharmaceutically acceptable salt thereof. In another embodiment, the cancer is characterized by amplification or overexpression of PARP. In some embodiments, the cancer is ovarian cancer or breast cancer, characterized by amplification or overexpression of PARP.

In some embodiments, provided herein is a method of treating a disease or disorder associated with PARP in a patient, comprising administering to the patient a therapeutically effective amount of a provided compound or a pharmaceutically acceptable salt thereof. In some embodiments, the disease or disorder associated with PARP is associated with an amplification of the PARP gene and/or overexpression of PARP.

Compounds of the present invention may also be useful for treatment of inflammatory conditions such as inflammatory bowel disorders.

Compounds of the present invention may also be useful for treatment of diabetes and/or as diabetes-prevention therapeutics.

Compounds of the present invention may also be useful for treatment of endotoxic shock or septic shock.

Compounds of the present invention may also be useful for treatment of peripheral nerve injuries, and the resultant pathological pain syndrome known as neuropathic pain, such as that induced by chronic constriction injury (CCI) of the common sciatic nerve and in which transsynaptic alteration of spinal cord dorsal horn characterized by hyperchromatosis of cytoplasm and nucleoplasm (so-called “dark” neurons) occurs (MAO et al., Pain, vol. 72, p: 355-366, 1997).

PARP inhibitors have also been used to extend the lifespan and proliferative capacity of cells including treatment of diseases such as skin aging (U.S. Pat. No. 5,589,483). Compounds of the present invention may also be useful for treatment Alzheimer's disease, atherosclerosis, osteoarthritis, osteoporosis, muscular dystrophy, degenerative diseases of skeletal muscle involving replicative senescence, age-related macular degeneration, immune senescence, AIDS, and other immune senescence diseases; and to alter gene expression of senescent cells.

Compounds of the present invention may also be useful for treatment of tissue damage resulting from cell damage or death due to necrosis or apoptosis, neural tissue damage resulting from ischemia and reperfusion injury, neurological disorders and neurodegenerative diseases; to prevent or treat vascular stroke; to treat or prevent cardiovascular disorders; to treat other conditions and/or disorders such as age-related macular degeneration, AIDS and other immune senescence diseases, arthritis, atherosclerosis, cachexia, cancer, degenerative diseases of skeletal muscle involving replicative senescence, diabetes, head trauma, immune senescence, inflammatory disorders like inflammatory bowel disorders such as colitis and Crohn's disease, muscular dystrophy, osteoarthritis, osteoporosis, chronic and/or acute pain (such as neuropathic pain), renal failure, retinal ischemia, septic shock (such as endotoxic shock), and skin aging; to extend the lifespan and proliferative capacity of cells; to alter gene expression of senescent cells; or to radiosensitize tumor cells.

Examples of neurodegenerative diseases that are treatable by the method of the present invention include, without limitation, trigeminal neuralgia; glossopharyngeal neuralgia; Bell's Palsy; myasthenia gravis; muscular dystrophy; amyotrophic lateral sclerosis; progressive muscular atrophy; progressive bulbar inherited muscular atrophy; herniated, ruptured or prolapsed invertebrate disk syndromes; cervical spondylosis; plexus disorders; thoracic outlet destruction syndromes; peripheral neuropathies such as those caused by lead, dapsone, ticks, porphyria, or Guillain-Barre syndrome; Alzheimer's disease; Huntington's Disease and Parkinson's disease.

Examples of cardiovascular disorders that can either cause ischemia or are caused by reperfusion of the heart include, but are not limited to, coronary artery disease, angina pectoris, myocardial infarction, cardiovascular tissue damage caused by cardiac arrest, cardiovascular tissue damage caused by cardiac bypass, cardiogenic shock, and related conditions that would be known by those of ordinary skill in the art or which involve dysfunction of or tissue damage to the heart or vasculature, especially, tissue damage related to PARP activation.

Compounds of the present invention may also be used to treat cancer, protein folding/misfolding disease, diabetes mellitus, Wolcott-Rallison syndrome, ischemia/reperfusion injury, stroke, neurodegeneration, atherosclerosis, neoplasia, hypoxia, or hypoglycemia.

Cancers which may be treated with compounds herein includes colon adenocarcinoma, esophagus adenocarcinoma, liver hepatocellular carcinoma, squamous cell carcinoma, pancreas adenocarcinoma, islet cell tumor, rectum adenocarcinoma, gastrointestinal stromal tumor, stomach adenocarcinoma, adrenal cortical carcinoma, follicular carcinoma, papillary carcinoma, breast cancer, ductal carcinoma, lobular carcinoma, intraductal carcinoma, mucinous carcinoma, Phyllodes tumor, Ewing's sarcoma, ovarian adenocarcinoma, endometrium adenocarcinoma, granulose cell tumor, mucinous cystadenocarcinoma, cervix adenocarcinoma, vulva squamous cell carcinoma, basal cell carcinoma, prostate adenocarcinoma, giant cell tumor of bone, bone osteosarcoma, larynx carcinoma, lung adenocarcinoma, kidney carcinoma, urinary bladder carcinoma, Wilm's tumor, and lymphoma.

Protein folding/misfolding diseases include Huntington's disease, spinobulbar muscular atrophy (Kennedy disease), Machado-Joseph disease, dentatorubral-pallidoluysian atrophy (Haw River Syndrome), spinocerebellar ataxia, Alzheimer's disease, Parkinson's disease, amyotrophic lateral sclerosis (ALS), Creutzfeldt-Jakob disease, bovine spongiform encephalopathy (BSE), light chain amyloidosis (AL), heavy chain amyloidosis (AH), secondary amyloidosis (AA), aortic medial amyloidosis, ApoA1, ApoAII, ad ApoAIV amyloidosis, insulin amyloidosis, cerebral 3-amyloid angiopathy, retinal ganglion cell degeneration in glaucoma, prion diseases, cataracts, tauopathies, frontotemporal lobar degeneration (FLTD), FTLD-FUS, amyotrophic lateral sclerosis (ALS/Lou Gehrig's disease), cerebral autosomal dominant arteriopathy with subcortical infarcts and leukoencephalopathy (CADASIL), Alexander disease, familial amyloidotic neuropathy, senile systemic amyloidosis, serpinopthies, Cystic Fibrosis, and Sickle Cell Disease.

In some embodiments, provided herein is a method of treating a disease or disorder associated with tumors that test positive for homologous recombination deficiency (HRD).

In some embodiments, the disease or disorder associated with PARP is lung squamous cell carcinoma, lung adenocarcinoma, pancreatic adenocarcinoma, breast invasive carcinoma, uterine carcinosarcoma, ovarian serous cystadenocarcinoma, stomach adenocarcinoma, esophageal carcinoma, bladder urothelial carcinoma, mesothelioma, or sarcoma.

In some embodiments, the disease or disorder associated with PARP is lung adenocarcinoma, breast invasive carcinoma, uterine carcinosarcoma, ovarian serous cystadenocarcinoma, or stomach adenocarcinoma.

In some embodiments, the disease or disorder associated with PARP is an adenocarcinoma, carcinoma, or cystadenocarcinoma.

In some embodiments, the disease or disorder associated with PARP is uterine cancer, ovarian cancer, stomach cancer, esophageal cancer, lung cancer, bladder cancer, pancreatic cancer, or breast cancer.

In some embodiments, the disease or disorder associated with PARP is a cancer.

In some embodiments, the cancer is characterized by amplification or overexpression of PARP. In some embodiments, the cancer is ovarian cancer or breast cancer, characterized by amplification or overexpression of PARP.

In some embodiments, the breast cancer is chemotherapy or radiotherapy resistant breast cancer, endocrine resistant breast cancer, trastuzumab resistant breast cancer, or breast cancer demonstrating primary or acquired resistance to PARP inhibition. In some embodiments, the breast cancer is advanced or metastatic breast cancer.

Examples of cancers that are treatable using the compounds of the present disclosure include, but are not limited to, bone cancer, pancreatic cancer, skin cancer, cancer of the head or neck, cutaneous or intraocular malignant melanoma, uterine cancer, ovarian cancer, rectal cancer, cancer of the anal region, stomach cancer, testicular cancer, uterine cancer, carcinoma of the fallopian tubes, carcinoma of the endometrium, endometrial cancer, carcinoma of the cervix, carcinoma of the vagina, carcinoma of the vulva, Hodgkin's Disease, non-Hodgkin's lymphoma, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, chronic or acute leukemias including acute myeloid leukemia, chronic myeloid leukemia, acute lymphoblastic leukemia, chronic lymphocytic leukemia, solid tumors of childhood, lymphocytic lymphoma, cancer of the bladder, cancer of the kidney or urethra, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, T-cell lymphoma, environmentally induced cancers including those induced by asbestos, and combinations of said cancers. The compounds of the present disclosure are also useful for the treatment of metastatic cancers.

In some embodiments, cancers treatable with compounds of the present disclosure include melanoma (e.g., metastatic malignant melanoma, BRAF and HSP90 inhibition-resistant melanoma), renal cancer (e.g., clear cell carcinoma), prostate cancer (e.g., hormone refractory prostate adenocarcinoma), breast cancer, colon cancer, lung cancer (e.g., non-small cell lung cancer and small cell lung cancer), squamous cell head and neck cancer, urothelial cancer (e.g., bladder) and cancers with high microsatellite instability (MSI^{high}). Additionally, the disclosure includes refractory or recurrent malignancies whose growth may be inhibited using the compounds of the disclosure.

In some embodiments, cancers that are treatable using the compounds of the present disclosure include, but are not limited to, solid tumors (e.g., prostate cancer, colon cancer, esophageal cancer, endometrial cancer, ovarian cancer, uterine cancer, renal cancer, hepatic cancer, pancreatic cancer, gastric cancer, breast cancer, lung cancer, cancers of the head and neck, thyroid cancer, glioblastoma, sarcoma, bladder cancer, etc.), hematological cancers (e.g., lymphoma, leukemia such as acute lymphoblastic leukemia (ALL), acute myelogenous leukemia (AML), chronic lymphocytic leukemia (CLL), chronic myelogenous leukemia (CML), DLBCL, mantle cell lymphoma, Non-Hodgkin lymphoma (including relapsed or refractory NHL and recurrent follicular), Hodgkin lymphoma or multiple myeloma) and combinations of said cancers.

In some embodiments, cancers that are treatable using the compounds of the present disclosure include, but are not limited to, cholangiocarcinoma, bile duct cancer, triple negative breast cancer, rhabdomyosarcoma, small cell lung cancer, leiomyosarcoma, hepatocellular carcinoma, Ewing's sarcoma, brain cancer, brain tumor, astrocytoma, neuroblastoma, neurofibroma, basal cell carcinoma, chondrosarcoma, epithelioid sarcoma, eye cancer, Fallopian tube cancer, gastrointestinal cancer, gastrointestinal stromal tumors, hairy cell leukemia, intestinal cancer, islet cell cancer, oral cancer, mouth cancer, throat cancer, laryngeal cancer, lip cancer, mesothelioma, neck cancer, nasal cavity cancer, ocular cancer, ocular melanoma, pelvic cancer, rectal cancer, renal cell carcinoma, salivary gland cancer, sinus cancer, spinal cancer, tongue cancer, tubular carcinoma, urethral cancer, and ureteral cancer.

In some embodiments, the compounds of the present disclosure can be used to treat sickle cell disease and sickle cell anemia.

In some embodiments, diseases and indications that are treatable using the compounds of the present disclosure include, but are not limited to hematological cancers, sarcomas, lung cancers, gastrointestinal cancers, genitourinary tract cancers, liver cancers, bone cancers, nervous system cancers, gynecological cancers, and skin cancers.

Exemplary hematological cancers include lymphomas and leukemias such as acute lymphoblastic leukemia (ALL), acute myelogenous leukemia (AML), acute promyelocytic leukemia (APL), chronic lymphocytic leukemia (CLL), chronic myelogenous leukemia (CML), diffuse large B-cell lymphoma (DLBCL), mantle cell lymphoma, Non-Hodgkin lymphoma (including relapsed or refractory NHL and recurrent follicular), Hodgkin lymphoma, myeloproliferative diseases (e.g., primary myelofibrosis (PMF), polycythemia vera (PV), and essential thrombocytosis (ET)), myelodysplasia syndrome (MDS), T-cell acute lymphoblastic lymphoma (T-ALL) and multiple myeloma (MM).

Exemplary sarcomas include chondrosarcoma, Ewing's sarcoma, osteosarcoma, rhabdomyosarcoma, angiosarcoma, fibrosarcoma, liposarcoma, myxoma, rhabdomyoma, rhabdosarcoma, fibroma, lipoma, harmatoma, and teratoma.

Exemplary lung cancers include non-small cell lung cancer (NSCLC), small cell lung cancer (SCLC), bronchogenic carcinoma, squamous cell, undifferentiated small cell, undifferentiated large cell, adenocarcinoma, alveolar (bronchiolar) carcinoma, bronchial adenoma, chondromatous hamartoma, and mesothelioma.

Exemplary gastrointestinal cancers include cancers of the esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Kaposi's sarcoma, leiomyoma, hemangioma, lipoma, neurofibroma, fibroma), large bowel (adenocarcinoma, tubular adenoma, villous adenoma, hamartoma, leiomyoma), and colorectal cancer.

Exemplary genitourinary tract cancers include cancers of the kidney (adenocarcinoma, Wilm's tumor [nephroblastoma]), bladder and urethra (squamous cell carcinoma, transitional cell carcinoma, adenocarcinoma), prostate (adenocarcinoma, sarcoma), and testis (seminoma, teratoma, embryonal carcinoma, teratocarcinoma, choriocarcinoma, sarcoma, interstitial cell carcinoma, fibroma, fibroadenoma, adenomatoid tumors, lipoma).

Exemplary liver cancers include hepatoma (hepatocellular carcinoma), cholangiocarcinoma, hepatoblastoma, angiosarcoma, hepatocellular adenoma, and hemangioma.

Exemplary bone cancers include, for example, osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma, and giant cell tumors

Exemplary nervous system cancers include cancers of the skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma (pinealoma), glioblastoma, glioblastoma multiform, oligodendroglioma, schwannoma, retinoblastoma, congenital tumors), and spinal cord (neurofibroma, meningioma, glioma, sarcoma), as well as neuroblastoma and Lhermitte-Duclos disease.

Exemplary gynecological cancers include cancers of the uterus (endometrial carcinoma), cervix (cervical carcinoma, pre-tumor cervical dysplasia), ovaries (ovarian carcinoma (serous cystadenocarcinoma, mucinous cystadenocarcinoma, unclassified carcinoma), granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), and fallopian tubes (carcinoma).

Exemplary skin cancers include melanoma, basal cell carcinoma, Merkel cell carcinoma, squamous cell carcinoma, Kaposi's sarcoma, moles dysplastic nevi, lipoma, angioma, dermatofibroma, and keloids. In some embodiments, diseases and indications that are treatable using the compounds of the present disclosure include, but are not limited to, sickle cell disease (e.g., sickle cell anemia), triple-negative breast cancer (TNBC), myelodysplastic syndromes, testicular cancer, bile duct cancer, esophageal cancer, and urothelial carcinoma.

In some embodiments, the method comprises killing one or more cancer cells in a patient with cancer. As such, the methods of the disclosure may be used to treat a tumor derived from a neoplasm or a cancer. In some embodiments, the tumors is harboring a mutation. In some embodiments, the tumor is harboring PTEN inactivation. In some embodiments, the tumor is harboring one or more PI3K pathway mutations. In some embodiments, the tumor is harboring one or more KRAS pathway mutations. In some embodiments, the tumor is harboring one or more Germline alterations. In some embodiments, the tumor is harboring a somatic deleterious BRCA alteration. In some embodiments, the tumor is a homologous recombination deficient (HRD) positive tumor. In some embodiments, the tumor is harboring a suspected germline BRCA deleterious alteration. In some embodiments, the tumor is harboring a Germline or somatic deleterious BRCA alteration. In some embodiments, the tumor is harboring a Germline or somatic deleterious BRCA alteration and/or homologous recombination deficient (HRD) positive tumor. In some embodiments, the tumor is harboring a Germline or suspected germline BRCA deleterious alteration.

It is believed that a provided compound or a pharmaceutically acceptable salt thereof may possess satisfactory pharmacological profile and promising biopharmaceutical properties, such as toxicological profile, metabolism and pharmacokinetic properties, solubility, and permeability. It will be understood that determination of appropriate biopharmaceutical properties is within the knowledge of a person skilled in the art, e.g., determination of cytotoxicity in cells or inhibition of certain targets or channels to determine potential toxicity.

As used herein, the terms “treatment,” “treat,” and “treating” refer to reversing, alleviating, delaying the onset of, or inhibiting the progress of a disease or disorder, or one or more symptoms thereof, as described herein. In some embodiments, treatment may be administered after one or more symptoms have developed. In other embodiments, treatment may be administered in the absence of symptoms. For example, treatment may be administered to a susceptible individual prior to the onset of symptoms (e.g., in light of a history of symptoms and/or in light of genetic or other susceptibility factors). Treatment may also be continued after symptoms have resolved, for example to prevent or delay their recurrence.

The terms “individual” or “patient,” used interchangeably, refer to any animal, including mammals, preferably mice, rats, other rodents, rabbits, dogs, cats, swine, cattle, sheep, horses, or primates, and most preferably humans.

The phrase “therapeutically effective amount” refers to the amount of active compound or pharmaceutical agent that elicits the biological or medicinal response in a tissue, system, animal, individual or human that is being sought by a researcher, veterinarian, medical doctor or other clinician.

In some embodiments, the compounds of the invention are useful in preventing or reducing the risk of developing any of the diseases referred to herein; e.g., preventing or reducing the risk of developing a disease, condition or disorder in an individual who may be predisposed to the disease, condition or disorder but does not yet experience or display the pathology or symptomatology of the disease.

Co-Administration with One or More Other Therapeutic Agent(s)

Depending upon the particular condition, or disease, to be treated, additional therapeutic agents that are normally administered to treat that condition, can also be present in the compositions of this invention. As used herein, additional therapeutic agents that are normally administered to treat a particular disease, or condition, are known as “appropriate for the disease, or condition, being treated.”

In some embodiments, the present invention provides a method of treating a disclosed disease or condition comprising administering to a patient in need thereof an effective amount of a compound disclosed herein or a pharmaceutically acceptable salt thereof and co-administering simultaneously or sequentially an effective amount of one or more additional therapeutic agents, such as those described herein. In some embodiments, the method includes co-administering one additional therapeutic agent. In some embodiments, the method includes co-administering two additional therapeutic agents. In some embodiments, the combination of the disclosed compound and the additional therapeutic agent or agents acts synergistically.

A compound of the current invention can also be used in combination with known therapeutic processes, for example, the administration of hormones or radiation. In some embodiments, a provided compound is used as a radiosensitizer, especially for the treatment of tumors which exhibit poor sensitivity to radiotherapy.

A compound of the current invention can be administered alone or in combination with one or more other therapeutic compounds, possible combination therapy taking the form of fixed combinations or the administration of a compound of the invention and one or more other therapeutic compounds being staggered or given independently of one another, or the combined administration of fixed combinations and one or more other therapeutic compounds. A compound of the current invention can besides, or in addition, be administered especially for tumor therapy in combination with chemotherapy, radiotherapy, immunotherapy, phototherapy, surgical intervention, or a combination of these. Long-term therapy is equally possible, as is adjuvant therapy in the context of other treatment strategies, as described above. Other possible treatments are therapy to maintain the patient's status after tumor regression, or even chemopreventive therapy, for example in patients at risk.

One or more other therapeutic agent(s) can be administered separately from a compound or composition of the invention, as part of a multiple dosage regimen. Alternatively, one or more other therapeutic agent(s) may be part of a single dosage form, mixed together with a compound of this invention in a single composition. If administered as a multiple dosage regime, one or more other therapeutic agent(s) and a compound or composition of the invention can be administered simultaneously, sequentially or within a period of time from one another, for example within 1, 2, 3, 4, 5, 6, 7, 8, 9, 10, 11, 12, 13, 14, 15, 16, 17, 18, 18, 20, 21, 22, 23, or 24 hours from one another. In some embodiments, one or more other therapeutic agent(s) and a compound or composition of the invention are administered as a multiple dosage regimen within greater than 24 hours apart.

As used herein, the term “combination,” “combined,” and related terms refers to the simultaneous or sequential administration of therapeutic agents in accordance with this invention. For example, a compound of the present invention can be administered with one or more other therapeutic agent(s) simultaneously or sequentially in separate unit dosage forms or together in a single unit dosage form. Accordingly, the present invention provides a single unit dosage form comprising a compound of the current invention, one or more other therapeutic agent(s), and a pharmaceutically acceptable carrier, adjuvant, or vehicle.

The amount of a compound of the invention and one or more other therapeutic agent(s) (in those compositions which comprise an additional therapeutic agent as described above) that can be combined with the carrier materials to produce a single dosage form varies depending upon the host treated and the particular mode of administration. Preferably, a composition of the invention should be formulated so that a dosage of between 0.01-100 mg/kg body weight/day of a compound of the invention can be administered.

In those compositions which comprise one or more other therapeutic agent(s), the one or more other therapeutic agent(s) and a compound of the invention can act synergistically. Therefore, the amount of the one or more other therapeutic agent(s) in such compositions may be less than that required in a monotherapy utilizing only that therapeutic agent. In such compositions a dosage of between 0.01-1,000 g/kg body weight/day of the one or more other therapeutic agent(s) can be administered.

The amount of one or more other therapeutic agent(s) present in the compositions of this invention may be no more than the amount that would normally be administered in a composition comprising that therapeutic agent as the only active agent. Preferably the amount of one or more other therapeutic agent(s) in the presently disclosed compositions ranges from about 50% to 100% of the amount normally present in a composition comprising that agent as the only therapeutically active agent. In some embodiments, one or more other therapeutic agent(s) is administered at a dosage of about 50%, about 55%, about 60%, about 65%, about 70%, about 75%, about 80%, about 85%, about 90%, or about 95% of the amount normally administered for that agent. As used herein, the phrase “normally administered” means the amount an FDA approved therapeutic agent is provided for dosing per the FDA label insert.

The compounds of this invention, or pharmaceutical compositions thereof, can also be incorporated into compositions for coating an implantable medical device, such as prostheses, artificial valves, vascular grafts, stents and catheters. Vascular stents, for example, have been used to overcome restenosis (re-narrowing of the vessel wall after injury). However, patients using stents or other implantable devices risk clot formation or platelet activation. These unwanted effects may be prevented or mitigated by pre-coating the device with a pharmaceutically acceptable composition comprising a kinase inhibitor. Implantable devices coated with a compound of this invention are another embodiment of the present invention.

In some embodiments, one or more other therapeutic agent is a ATM kinase inhibitor.

In some embodiments, one or more other therapeutic agent is a ATR kinase inhibitor.

In some embodiments, one or more other therapeutic agent is a CHK1 kinase inhibitor.

In some embodiments, one or more other therapeutic agent is a WEE1 inhibitor.

In some embodiments, one or more other therapeutic agent is an immune checkpoint inhibitor. In some embodiments, the immune checkpoint inhibitor is selected from a PD-1 antagonist, a PD-L1 antagonist, and a CTLA-4 antagonist, an OX40 agonist, a CD137 (also called 4-1BB) agonist, a CD27 agonist

### Exemplary Other Therapeutic Agents

In some embodiments, one or more other therapeutic agent is a Poly ADP ribose polymerase (PARP) inhibitor. In some embodiments, a PARP inhibitor is selected from olaparib (LYNPARZA®, AstraZeneca); rucaparib (RUBRACA®, Clovis Oncology); niraparib (ZEJULA®, Tesaro); talazoparib (MDV3800/BMN 673/LT00673, Medivation/Pfizer/Biomarin); veliparib (ABT-888, AbbVie); and BGB-290 (BeiGene, Inc.).

In some embodiments, one or more other therapeutic agent is a histone deacetylase (HDAC) inhibitor. In some embodiments, an HDAC inhibitor is selected from vorinostat (ZOLINZA®, Merck); romidepsin (ISTODAX®, Celgene); panobinostat (FARYDAK®, Novartis); belinostat (BELEODAQ®, Spectrum Pharmaceuticals); entinostat (SNDX-275, Syndax Pharmaceuticals) (NCT00866333); and chidamide (EPIDAZA®, HBI-8000, Chipscreen Biosciences, China).

In some embodiments, one or more other therapeutic agent is a CDK inhibitor, such as a CDK4/CDK6 inhibitor. In some embodiments, a CDK 4/6 inhibitor is selected from palbociclib (IBRANCE®, Pfizer); ribociclib (KISQALI®, Novartis); abemaciclib (Ly2835219, Eli Lilly); and trilaciclib (G1T28, G1 Therapeutics).

In some embodiments, one or more other therapeutic agent is a phosphatidylinositol 3 kinase (PI3K) inhibitor. In some embodiments, a PI3K inhibitor is selected from idelalisib (ZYDELIG®, Gilead), alpelisib (BYL719, Novartis), taselisib (GDC-0032, Genentech/Roche); pictilisib (GDC-0941, Genentech/Roche); copanlisib (BAY806946, Bayer); duvelisib (formerly IPI-145, Infinity Pharmaceuticals); PQR309 (Piqur Therapeutics, Switzerland); and TGR1202 (formerly RP5230, TG Therapeutics).

In some embodiments, one or more other therapeutic agent is a platinum-based therapeutic, also referred to as platins. Platins cause cross-linking of DNA, such that they inhibit DNA repair and/or DNA synthesis, mostly in rapidly reproducing cells, such as cancer cells. In some embodiments, a platinum-based therapeutic is selected from cisplatin (PLATINOL®, Bristol-Myers Squibb); carboplatin (PARAPLATIN®, Bristol-Myers Squibb; also, Teva; Pfizer); oxaliplatin (ELOXITIN® Sanofi-Aventis); nedaplatin (AQUPLAk, Shionogi), picoplatin (Poniard Pharmaceuticals); and satraplatin (JM-216, Agennix).

In some embodiments, one or more other therapeutic agent is a taxane compound, which causes disruption of microtubules, which are essential for cell division. In some embodiments, a taxane compound is selected from paclitaxel (TAXOL®, Bristol-Myers Squibb), docetaxel (TAXOTERE®, Sanofi-Aventis; DOCEFREZ®, Sun Pharmaceutical), albumin-bound paclitaxel (ABRAXANE®; Abraxis/Celgene), cabazitaxel (JEVTANA®, Sanofi-Aventis), and SID530 (SK Chemicals, Co.) (NCT00931008).

In some embodiments, one or more other therapeutic agent is a nucleoside inhibitor, or a therapeutic agent that interferes with normal DNA synthesis, protein synthesis, cell replication, or will otherwise inhibit rapidly proliferating cells.

In some embodiments, a nucleoside inhibitor is selected from trabectedin (guanidine alkylating agent, YONDELIS®, Janssen Oncology), mechlorethamine (alkylating agent, VALCHLOR®, Aktelion Pharmaceuticals); vincristine (ONCOVIN®, Eli Lilly; VINCASAR®, Teva Pharmaceuticals; MARQIBO®, Talon Therapeutics); temozolomide (prodrug to alkylating agent 5-(3-methyltriazen-1-yl)-imidazole-4-carboxamide (MTIC) TEMODAR®, Merck); cytarabine injection (ara-C, antimetabolic cytidine analog, Pfizer); lomustine (alkylating agent, CEENU®, Bristol-Myers Squibb; GLEOSTINE®, NextSource Biotechnology); azacitidine (pyrimidine nucleoside analog of cytidine, VIDAZA®, Celgene); omacetaxine mepesuccinate (cephalotaxine ester) (protein synthesis inhibitor, SYNRIBO®; Teva Pharmaceuticals); asparaginase Erwinia chrysanthemi (enzyme for depletion of asparagine, ELSPAR®, Lundbeck; ERWINAZE®, EUSA Pharma); eribulin mesylate (microtubule inhibitor, tubulin-based antimitotic, HALAVEN®, Eisai); cabazitaxel (microtubule inhibitor, tubulin-based antimitotic, JEVTANA®, Sanofi-Aventis); capacetrine (thymidylate synthase inhibitor, XELODA®, Genentech); bendamustine (bifunctional mechlorethamine derivative, believed to form interstrand DNA cross-links, TREANDA®, Cephalon/Teva); ixabepilone (semi-synthetic analog of epothilone B, microtubule inhibitor, tubulin-based antimitotic, IXEMPRA®, Bristol-Myers Squibb); nelarabine (prodrug of deoxyguanosine analog, nucleoside metabolic inhibitor, ARRANON®, Novartis); clorafabine (prodrug of ribonucleotide reductase inhibitor, competitive inhibitor of deoxycytidine, CLOLAR®, Sanofi-Aventis); and trifluridine and tipiracil (thymidine-based nucleoside analog and thymidine phosphorylase inhibitor, LONSURF®, Taiho Oncology).

In some embodiments, one or more other therapeutic agent is a kinase inhibitor or VEGF-R antagonist. Approved VEGF inhibitors and kinase inhibitors useful in the present invention include: bevacizumab (AVASTIN®, Genentech/Roche) an anti-VEGF monoclonal antibody; ramucirumab (CYRAMZA®, Eli Lilly), an anti-VEGFR-2 antibody and ziv-aflibercept, also known as VEGF Trap (ZALTRAP®; Regeneron/Sanofi). VEGFR inhibitors, such as regorafenib (STIVARGA®, Bayer); vandetanib (CAPRELSA®, AstraZeneca); axitinib (INLYTA®, Pfizer); and lenvatinib (LENVIMA®, Eisai); Raf inhibitors, such as sorafenib (NEXAVAR®, Bayer AG and Onyx); dabrafenib (TAFINLAR®, Novartis); and vemurafenib (ZELBORAF®, Genentech/Roche); MEK inhibitors, such as cobimetanib (COTELLIC®, Exelexis/Genentech/Roche); trametinib (MEKINIST®, Novartis); Bcr-Abl tyrosine kinase inhibitors, such as imatinib (GLEEVEC®, Novartis); nilotinib (TASIGNA®, Novartis); dasatinib (SPRYCEL®, BristolMyersSquibb); bosutinib (BOSULIF®, Pfizer); and ponatinib (INCLUSIG®, Ariad Pharmaceuticals); Her2 and EGFR inhibitors, such as gefitinib (IRESSA®, AstraZeneca); erlotinib (TARCEEVA®, Genentech/Roche/Astellas); lapatinib (TYKERB®, Novartis); afatinib (GILOTRIF®, Boehringer Ingelheim); osimertinib (targeting activated EGFR, TAGRISSO®, AstraZeneca); and brigatinib (ALUNBRIG®, Ariad Pharmaceuticals); c-Met and VEGFR2 inhibitors, such as cabozanitib (COMETRIQ®, Exelexis); and multikinase inhibitors, such as sunitinib (SUTENT®, Pfizer); pazopanib (VOTRIENT®, Novartis); ALK inhibitors, such as crizotinib (XALKORI®, Pfizer); ceritinib (ZYKADIA®, Novartis); and alectinib (ALECENZa®, Genentech/Roche); Bruton's tyrosine kinase inhibitors, such as ibrutinib (IMBRUVICA®, Pharmacyclics/Janssen); and Flt3 receptor inhibitors, such as midostaurin (RYDAPT®, Novartis).

Other kinase inhibitors and VEGF-R antagonists that are in development and may be used in the present invention include tivozanib (Aveo Pharmaecuticals); vatalanib (Bayer/Novartis); lucitanib (Clovis Oncology); dovitinib (TKI258, Novartis); Chiauanib (Chipscreen Biosciences); CEP-11981 (Cephalon); linifanib (Abbott Laboratories); neratinib (HKI-272, Puma Biotechnology); radotinib (SUPECT®, IY5511, Il-Yang Pharmaceuticals, S. Korea); ruxolitinib (JAKAFI®, Incyte Corporation); PTC299 (PTC Therapeutics); CP-547,632 (Pfizer); foretinib (Exelexis, GlaxoSmithKline); quizartinib (Daiichi Sankyo) and motesanib (Amgen/Takeda).

In some embodiments, one or more other therapeutic agent is an mTOR inhibitor, which inhibits cell proliferation, angiogenesis and glucose uptake. In some embodiments, an mTOR inhibitor is everolimus (AFINITOR®, Novartis); temsirolimus (TORISEL®, Pfizer); and sirolimus (RAPAMUNE®, Pfizer).

In some embodiments, one or more other therapeutic agent is a proteasome inhibitor. Approved proteasome inhibitors useful in the present invention include bortezomib (VELCADE®, Takeda); carfilzomib (KYPROLIS®, Amgen); and ixazomib (NINLARO®, Takeda).

In some embodiments, one or more other therapeutic agent is a growth factor antagonist, such as an antagonist of platelet-derived growth factor (PDGF), or epidermal growth factor (EGF) or its receptor (EGFR). Approved PDGF antagonists which may be used in the present invention include olaratumab (LARTRUVO®; Eli Lilly). Approved EGFR antagonists which may be used in the present invention include cetuximab (ERBITUX®, Eli Lilly); necitumumab (PORTRAZZA®, Eli Lilly), panitumumab (VECTIBIX®, Amgen); and osimertinib (targeting activated EGFR, TAGRISSO®, AstraZeneca).

In some embodiments, one or more other therapeutic agent is an aromatase inhibitor. In some embodiments, an aromatase inhibitor is selected from exemestane (AROMASIN®, Pfizer); anastazole (ARIMIDEX®, AstraZeneca) and letrozole (FEMARA®, Novartis).

In some embodiments, one or more other therapeutic agent is an antagonist of the hedgehog pathway. Approved hedgehog pathway inhibitors which may be used in the present invention include sonidegib (ODOMZO®, Sun Pharmaceuticals); and vismodegib (ERIVEDGE®, Genentech), both for treatment of basal cell carcinoma.

In some embodiments, one or more other therapeutic agent is a folic acid inhibitor. Approved folic acid inhibitors useful in the present invention include pemetrexed (ALIMTA®, Eli Lilly).

In some embodiments, one or more other therapeutic agent is a CC chemokine receptor 4 (CCR4) inhibitor. CCR4 inhibitors being studied that may be useful in the present invention include mogamulizumab (POTELIGEO®, Kyowa Hakko Kirin, Japan).

In some embodiments, one or more other therapeutic agent is an isocitrate dehydrogenase (IDH) inhibitor. IDH inhibitors being studied which may be used in the present invention include AG120 (Celgene; NCT02677922); AG221 (Celgene, NCT02677922; NCT02577406); BAY1436032 (Bayer, NCT02746081); IDH305 (Novartis, NCT02987010).

In some embodiments, one or more other therapeutic agent is an arginase inhibitor. Arginase inhibitors being studied which may be used in the present invention include AEB1102 (pegylated recombinant arginase, Aeglea Biotherapeutics), which is being studied in Phase 1 clinical trials for acute myeloid leukemia and myelodysplastic syndrome (NCT02732184) and solid tumors (NCT02561234); and CB-1158 (Calithera Biosciences).

In some embodiments, one or more other therapeutic agent is a glutaminase inhibitor. Glutaminase inhibitors being studied which may be used in the present invention include CB-839 (Calithera Biosciences).

In some embodiments, one or more other therapeutic agent is an antibody that binds to tumor antigens, that is, proteins expressed on the cell surface of tumor cells. Approved antibodies that bind to tumor antigens which may be used in the present invention include rituximab (RITUXAN®, Genentech/BiogenIdec); ofatumumab (anti-CD20, ARZERRA®, GlaxoSmithKline); obinutuzumab (anti-CD20, GAZYVA®, Genentech), ibritumomab (anti-CD20 and Yttrium-90, ZEVALIN®, Spectrum Pharmaceuticals); daratumumab (anti-CD38, DARZALEX®, Janssen Biotech), dinutuximab (anti-glycolipid GD2, UNITUXIN®, United Therapeutics); trastuzumab (anti-HER2, HERCEPTIN®, Genentech); ado-trastuzumab emtansine (anti-HER2, fused to emtansine, KADCYLA®, Genentech); and pertuzumab (anti-HER2, PERJETA®, Genentech); and brentuximab vedotin (anti-CD30-drug conjugate, ADCETRIS®, Seattle Genetics).

In some embodiments, one or more other therapeutic agent is a topoisomerase inhibitor. Approved topoisomerase inhibitors useful in the present invention include irinotecan (ONIVYDE®, Merrimack Pharmaceuticals); topotecan (HYCAMTIN®, GlaxoSmithKline). Topoisomerase inhibitors being studied which may be used in the present invention include pixantrone (PIXUVRI®, CTI Biopharma).

In some embodiments, one or more other therapeutic agent is an inhibitor of anti-apoptotic proteins, such as BCL-2. Approved anti-apoptotics which may be used in the present invention include venetoclax (VENCLEXTA®, AbbVie/Genentech); and blinatumomab (BLINCYTO®, Amgen). Other therapeutic agents targeting apoptotic proteins which have undergone clinical testing and may be used in the present invention include navitoclax (ABT-263, Abbott), a BCL-2 inhibitor (NCT02079740).

In some embodiments, one or more other therapeutic agent is an androgen receptor inhibitor. Approved androgen receptor inhibitors useful in the present invention include enzalutamide (XTANDI®, Astellas/Medivation); approved inhibitors of androgen synthesis include abiraterone (ZYTIGA®, Centocor/Ortho); approved antagonist of gonadotropin-releasing hormone (GnRH) receptor (degaralix, FIRMAGON®, Ferring Pharmaceuticals).

In some embodiments, one or more other therapeutic agent is a selective estrogen receptor modulator (SERM), which interferes with the synthesis or activity of estrogens. Approved SERMs useful in the present invention include raloxifene (EVISTA®, Eli Lilly).

In some embodiments, one or more other therapeutic agent is an inhibitor of bone resorption. An approved therapeutic which inhibits bone resorption is Denosumab (XGEVA®, Amgen), an antibody that binds to RANKL, prevents binding to its receptor RANK, found on the surface of osteoclasts, their precursors, and osteoclast-like giant cells, which mediates bone pathology in solid tumors with osseous metastases. Other approved therapeutics that inhibit bone resorption include bisphosphonates, such as zoledronic acid (ZOMETA®, Novartis).

In some embodiments, one or more other therapeutic agent is an inhibitor of interaction between the two primary p53 suppressor proteins, MDMX and MDM2. Inhibitors of p53 suppression proteins being studied which may be used in the present invention include ALRN-6924 (Aileron), a stapled peptide that equipotently binds to and disrupts the interaction of MDMX and MDM2 with p53. ALRN-6924 is currently being evaluated in clinical trials for the treatment of AML, advanced myelodysplastic syndrome (MDS) and peripheral T-cell lymphoma (PTCL) (NCT02909972; NCT02264613).

In some embodiments, one or more other therapeutic agent is an inhibitor of transforming growth factor-beta (TGF-beta or TGFB). Inhibitors of TGF-beta proteins being studied which may be used in the present invention include NIS793 (Novartis), an anti-TGF-beta antibody being tested in the clinic for treatment of various cancers, including breast, lung, hepatocellular, colorectal, pancreatic, prostate and renal cancer (NCT 02947165). In some embodiments, the inhibitor of TGF-beta proteins is fresolimumab (GC1008; Sanofi-Genzyme), which is being studied for melanoma (NCT00923169); renal cell carcinoma (NCT00356460); and non-small cell lung cancer (NCT02581787). Additionally, in some embodiments, the additional therapeutic agent is a TGF-beta trap, such as described in Connolly et al. (2012) Int'l J. Biological Sciences 8:964-978. One therapeutic compound currently in clinical trials for treatment of solid tumors is M7824 (Merck KgaA—formerly MSB0011459X), which is a bispecific, anti-PD-L1/TGF-β trap compound (NCT02699515); and (NCT02517398). M7824 is comprised of a fully human IgG1 antibody against PD-L1 fused to the extracellular domain of human TGF-beta receptor II, which functions as a TGF-β“trap.”

In some embodiments, one or more other therapeutic agent is selected from glembatumumab vedotin-monomethyl auristatin E (MMAE) (Celldex), an anti-glycoprotein NMB (gpNMB) antibody (CR011) linked to the cytotoxic MMAE. gpNMB is a protein overexpressed by multiple tumor types associated with cancer cells' ability to metastasize.

In some embodiments, one or more other therapeutic agents is an antiproliferative compound. Such antiproliferative compounds include, but are not limited to aromatase inhibitors; antiestrogens; topoisomerase I inhibitors; topoisomerase II inhibitors; microtubule active compounds; alkylating compounds; histone deacetylase inhibitors; compounds which induce cell differentiation processes; cyclooxygenase inhibitors; MMP inhibitors; mTOR inhibitors; antineoplastic antimetabolites; platin compounds; compounds targeting/decreasing a protein or lipid kinase activity and further anti-angiogenic compounds; compounds which target, decrease or inhibit the activity of a protein or lipid phosphatase; gonadorelin agonists; anti-androgens; methionine aminopeptidase inhibitors; matrix metalloproteinase inhibitors; bisphosphonates; biological response modifiers; antiproliferative antibodies; heparanase inhibitors; inhibitors of Ras oncogenic isoforms; telomerase inhibitors; proteasome inhibitors; compounds used in the treatment of hematologic malignancies; compounds which target, decrease or inhibit the activity of Flt-3; Hsp90 inhibitors such as 17-AAG (17-allylaminogeldanamycin, NSC330507), 17-DMAG (17-dimethylaminoethylamino-17-demethoxy-geldanamycin, NSC707545), IPI-504, TEMODAL CNF1010, CNF2024, CNF1010 from Conforma Therapeutics; temozolomide (TEMODAL®); kinesin spindle protein inhibitors, such as SB715992 or SB743921 from GlaxoSmithKline, or pentamidine/chlorpromazine from CombinatoRx; MEK inhibitors such as ARRY142886 from Array BioPharma, AZd₆244 from AstraZeneca, PD181461 from Pfizer and leucovorin.

In some embodiments, one or more other therapeutic agents is an USP1 inhibitor. In some embodiments the USP1 inhibitor is selected from KSQ-4279 (KSQ Therapeutics), TNG-348 (Tango Therapeutics) and Debio 0432 (formerly FT-3171, Deibopharm).

The term “aromatase inhibitor” as used herein relates to a compound which inhibits estrogen production, for instance, the conversion of the substrates androstenedione and testosterone to estrone and estradiol, respectively. The term includes, but is not limited to steroids, especially atamestane, exemestane and formestane and, in particular, non-steroids, especially aminoglutethimide, roglethimide, pyridoglutethimide, trilostane, testolactone, ketokonazole, vorozole, fadrozole, anastrozole and letrozole. Exemestane is marketed under the trade name AROMASIN™. Formestane is marketed under the trade name LENTARON™. Fadrozole is marketed under the trade name AFEMA™. Anastrozole is marketed under the trade name ARIMIDEX™. Letrozole is marketed under the trade names FEMARA™ or FEMAr™. Aminoglutethimide is marketed under the trade name ORIMETEN™. A combination of the invention comprising a chemotherapeutic agent which is an aromatase inhibitor is particularly useful for the treatment of hormone receptor positive tumors, such as breast tumors.

The term “antiestrogen” as used herein relates to a compound which antagonizes the effect of estrogens at the estrogen receptor level. The term includes, but is not limited to tamoxifen, fulvestrant, raloxifene and raloxifene hydrochloride. Tamoxifen is marketed under the trade name NOLVADEX™ Raloxifene hydrochloride is marketed under the trade name EVISTA™. Fulvestrant can be administered under the trade name FASLODEX™. A combination of the invention comprising a chemotherapeutic agent which is an antiestrogen is particularly useful for the treatment of estrogen receptor positive tumors, such as breast tumors.

The term “anti-androgen” as used herein relates to any substance which is capable of inhibiting the biological effects of androgenic hormones and includes, but is not limited to, bicalutamide (CASODEX™). The term “gonadorelin agonist” as used herein includes, but is not limited to abarelix, goserelin, and goserelin acetate. Goserelin can be administered under the trade name ZOLADEX™.

The term “topoisomerase I inhibitor” as used herein includes, but is not limited to topotecan, gimatecan, irinotecan, camptothecian and its analogues, 9-nitrocamptothecin and the macromolecular camptothecin conjugate PNU-166148. Irinotecan can be administered, e.g., in the form as it is marketed, e.g., under the trademark CAMPTOSAR™. Topotecan is marketed under the trade name HYCAMPTIN™.

The term “topoisomerase II inhibitor” as used herein includes, but is not limited to the anthracyclines such as doxorubicin (including liposomal formulation, such as CAELYX™), daunorubicin, epirubicin, idarubicin and nemorubicin, the anthraquinones mitoxantrone and losoxantrone, and the podophillotoxines etoposide and teniposide. Etoposide is marketed under the trade name ETOPOPHOS™ Teniposide is marketed under the trade name VM 26-Bristol Doxorubicin is marketed under the trade name ACRIBLASTIN™ or ADRIAMYCIN™. Epirubicin is marketed under the trade name FARMORUBICIN™. Idarubicin is marketed. under the trade name ZAVEDOS™. Mitoxantrone is marketed under the trade name NOVANTRON™.

The term “microtubule active agent” relates to microtubule stabilizing, microtubule destabilizing compounds and microtublin polymerization inhibitors including, but not limited to taxanes, such as paclitaxel and docetaxel; vinca alkaloids, such as vinblastine or vinblastine sulfate, vincristine or vincristine sulfate, and vinorelbine; discodermolides; cochicine and epothilones and derivatives thereof. Paclitaxel is marketed under the trade name TAXOL™. Docetaxel is marketed under the trade name TAXOTERE™. Vinblastine sulfate is marketed under the trade name VINBLASTIN R.P™. Vincristine sulfate is marketed under the trade name FARMISTIN™.

The term “alkylating agent” as used herein includes, but is not limited to, cyclophosphamide, ifosfamide, melphalan or nitrosourea (BCNU or Gliadel). Cyclophosphamide is marketed under the trade name CYCLOSTIN™. Ifosfamide is marketed under the trade name HOLOXAN™.

The term “histone deacetylase inhibitors” or “HDAC inhibitors” relates to compounds which inhibit the histone deacetylase and which possess antiproliferative activity. This includes, but is not limited to, suberoylanilide hydroxamic acid (SAHA).

The term “antineoplastic antimetabolite” includes, but is not limited to, 5-fluorouracil or 5-FU, capecitabine, gemcitabine, DNA demethylating compounds, such as 5-azacytidine and decitabine, methotrexate and edatrexate, and folic acid antagonists such as pemetrexed. Capecitabine is marketed under the trade name XELODA™. Gemcitabine is marketed under the trade name GEMZAR™.

The term “platin compound” as used herein includes, but is not limited to, carboplatin, cis-platin, cisplatinum and oxaliplatin. Carboplatin can be administered, e.g., in the form as it is marketed, e.g., under the trademark CARBOPLAT™. Oxaliplatin can be administered, e.g., in the form as it is marketed, e.g. under the trademark ELOXATIN™.

The term “compounds targeting/decreasing a protein or lipid kinase activity; or a protein or lipid phosphatase activity; or further anti-angiogenic compounds” as used herein includes, but is not limited to, protein tyrosine kinase and/or serine and/or threonine kinase inhibitors or lipid kinase inhibitors, such as a) compounds targeting, decreasing or inhibiting the activity of the platelet-derived growth factor-receptors (PDGFR), such as compounds which target, decrease or inhibit the activity of PDGFR, especially compounds which inhibit the PDGF receptor, such as an N-phenyl-2-pyrimidine-amine derivative, such as imatinib, SU101, SU6668 and GFB-111; b) compounds targeting, decreasing or inhibiting the activity of the fibroblast growth factor-receptors (FGFR); c) compounds targeting, decreasing or inhibiting the activity of the insulin-like growth factor receptor I (IGF-IR), such as compounds which target, decrease or inhibit the activity of IGF-IR, especially compounds which inhibit the kinase activity of IGF-I receptor, or antibodies that target the extracellular domain of IGF-I receptor or its growth factors; d) compounds targeting, decreasing or inhibiting the activity of the Trk receptor tyrosine kinase family, or ephrin B4 inhibitors; e) compounds targeting, decreasing or inhibiting the activity of the AxI receptor tyrosine kinase family; f) compounds targeting, decreasing or inhibiting the activity of the Ret receptor tyrosine kinase; g) compounds targeting, decreasing or inhibiting the activity of the Kit/SCFR receptor tyrosine kinase, such as imatinib; h) compounds targeting, decreasing or inhibiting the activity of the C-kit receptor tyrosine kinases, which are part of the PDGFR family, such as compounds which target, decrease or inhibit the activity of the c-Kit receptor tyrosine kinase family, especially compounds which inhibit the c-Kit receptor, such as imatinib; i) compounds targeting, decreasing or inhibiting the activity of members of the c-Abl family, their gene-fusion products (e.g., BCR-Abl kinase) and mutants, such as compounds which target decrease or inhibit the activity of c-Abl family members and their gene fusion products, such as an N-phenyl-2-pyrimidine-amine derivative, such as imatinib or nilotinib (AMN107); PD180970; AG957; NSC 680410; PD173955 from ParkeDavis; or dasatinib (BMS-354825); j) compounds targeting, decreasing or inhibiting the activity of members of the protein kinase C (PKC) and Raf family of serine/threonine kinases, members of the MEK, SRC, JAK/pan-JAK, FAK, PDK1, PKB/Akt, Ras/MAPK, PI3K, SYK, TYK2, BTK and TEC family, and/or members of the cyclin-dependent kinase family (CDK) including staurosporine derivatives, such as midostaurin; examples of further compounds include UCN-01, safingol, BAY 43-9006, Bryostatin 1, Perifosine; llmofosine; RO 318220 and RO 320432; GO 6976; 1sis 3521; LY333531/LY379196; isochinoline compounds; FTIs; PD184352 or QAN697 (a PI3K inhibitor) or AT7519 (CDK inhibitor); k) compounds targeting, decreasing or inhibiting the activity of protein-tyrosine kinase inhibitors, such as compounds which target, decrease or inhibit the activity of protein-tyrosine kinase inhibitors include imatinib mesylate (GLEEVEC™) or tyrphostin such as Tyrphostin A23/RG-50810; AG 99; Tyrphostin AG 213; Tyrphostin AG 1748; Tyrphostin AG 490; Tyrphostin B44; Tyrphostin B44 (+) enantiomer; Tyrphostin AG 555; AG 494; Tyrphostin AG 556, AG957 and adaphostin (4-{[(2,5-dihydroxyphenyl)methyl]amino}-benzoic acid adamantyl ester; NSC 680410, adaphostin); 1) compounds targeting, decreasing or inhibiting the activity of the epidermal growth factor family of receptor tyrosine kinases (EGFR1 ErbB2, ErbB3, ErbB4 as homo- or heterodimers) and their mutants, such as compounds which target, decrease or inhibit the activity of the epidermal growth factor receptor family are especially compounds, proteins or antibodies which inhibit members of the EGF receptor tyrosine kinase family, such as EGF receptor, ErbB2, ErbB3 and ErbB4 or bind to EGF or EGF related ligands, CP 358774, ZD 1839, ZM 105180; trastuzumab (HERCEPTIN™), cetuximab (ERBITUX™), Iressa, Tarceva, OSI-774, Cl-1033, EKB-569, GW-2016, ELI, E2.4, E2.5, E6.2, E6.4, E2.11, E6.3 or E7.6.3, and 7H-pyrrolo-[2,3-d]pyrimidine derivatives; m) compounds targeting, decreasing or inhibiting the activity of the c-Met receptor, such as compounds which target, decrease or inhibit the activity of c-Met, especially compounds which inhibit the kinase activity of c-Met receptor, or antibodies that target the extracellular domain of c-Met or bind to HGF, n) compounds targeting, decreasing or inhibiting the kinase activity of one or more JAK family members (JAK1/JAK2/JAK3/TYK2 and/or pan-JAK), including but not limited to PRT-062070, SB-1578, baricitinib, pacritinib, momelotinib, VX-509, AZD-1480, TG-101348, tofacitinib, and ruxolitinib; o) compounds targeting, decreasing or inhibiting the kinase activity of PI3 kinase (PI3K) including but not limited to ATU-027, SF-1126, DS-7423, PBI-05204, GSK-2126458, ZSTK-474, buparlisib, pictrelisib, PF-4691502, BYL-719, dactolisib, XL-147, XL-765, and idelalisib; and; and q) compounds targeting, decreasing or inhibiting the signaling effects of hedgehog protein (Hh) or smoothened receptor (SMO) pathways, including but not limited to cyclopamine, vismodegib, itraconazole, erismodegib, and IPI-926 (saridegib).

The term “PI3K inhibitor” as used herein includes, but is not limited to compounds having inhibitory activity against one or more enzymes in the phosphatidylinositol-3-kinase family, including, but not limited to PI3Kα, PI3Kγ, PI3Kδ, PI3Kβ, PI3K-C2α, PI3K-C2β, PI3K-C2γ, Vps34, p110-α, p110-β, p110-γ, p110-δ, p85-α, p85-β, p55-γ, p150, p101, and p87. Examples of PI3K inhibitors useful in this invention include but are not limited to ATU-027, SF-1126, DS-7423, PBI-05204, GSK-2126458, ZSTK-474, buparlisib, pictrelisib, PF-4691502, BYL-719, dactolisib, XL-147, XL-765, and idelalisib.

The term “Bcl-2 inhibitor” as used herein includes, but is not limited to compounds having inhibitory activity against B-cell lymphoma 2 protein (Bcl-2), including but not limited to ABT-199, ABT-731, ABT-737, apogossypol, Ascenta's pan-Bcl-2 inhibitors, curcumin (and analogs thereof), dual Bcl-2/Bcl-xL inhibitors (Infinity Pharmaceuticals/Novartis Pharmaceuticals), Genasense (G3139), HA14-1 (and analogs thereof; see WO2008118802), navitoclax (and analogs thereof, see U.S. Pat. No. 7,390,799), NH-1 (Shenayng Pharmaceutical University), obatoclax (and analogs thereof, see WO2004106328), S-001 (Gloria Pharmaceuticals), TW series compounds (Univ. of Michigan), and venetoclax. In some embodiments the Bcl-2 inhibitor is a small molecule therapeutic. In some embodiments the Bcl-2 inhibitor is a peptidomimetic.

The term “BTK inhibitor” as used herein includes, but is not limited to compounds having inhibitory activity against Bruton's Tyrosine Kinase (BTK), including, but not limited to AVL-292 and ibrutinib.

The term “SYK inhibitor” as used herein includes, but is not limited to compounds having inhibitory activity against spleen tyrosine kinase (SYK), including but not limited to PRT-062070, R-343, R-333, Excellair, PRT-062607, and fostamatinib.

Further examples of BTK inhibitory compounds, and conditions treatable by such compounds in combination with compounds of this invention can be found in WO2008039218 and WO2011090760, the entirety of which are incorporated herein by reference.

Further examples of SYK inhibitory compounds, and conditions treatable by such compounds in combination with compounds of this invention can be found in WO2003063794, WO2005007623, and WO2006078846, the entirety of which are incorporated herein by reference.

Further examples of PI3K inhibitory compounds, and conditions treatable by such compounds in combination with compounds of this invention can be found in WO2004019973, WO2004089925, WO2007016176, U.S. Pat. No. 8,138,347, WO2002088112, WO2007084786, WO2007129161, WO2006122806, WO2005113554, and WO2007044729 the entirety of which are incorporated herein by reference.

Further examples of JAK inhibitory compounds, and conditions treatable by such compounds in combination with compounds of this invention can be found in WO2009114512, WO2008109943, WO2007053452, WO2000142246, and WO2007070514, the entirety of which are incorporated herein by reference.

Further anti-angiogenic compounds include compounds having another mechanism for their activity, e.g., unrelated to protein or lipid kinase inhibition e.g., thalidomide (THALOMID™) and TNP-470.

Examples of proteasome inhibitors useful for use in combination with compounds of the invention include, but are not limited to bortezomib, disulfiram, epigallocatechin-3-gallate (EGCG), salinosporamide A, carfilzomib, ONX-0912, CEP-18770, and MLN9708.

Compounds which target, decrease or inhibit the activity of a protein or lipid phosphatase are e.g. inhibitors of phosphatase 1, phosphatase 2A, or CDC25, such as okadaic acid or a derivative thereof.

Compounds which induce cell differentiation processes include, but are not limited to, retinoic acid, α- γ- or δ-tocopherol or α- γ- or δ-tocotrienol.

The term cyclooxygenase inhibitor as used herein includes, but is not limited to, Cox-2 inhibitors, 5-alkyl substituted 2-arylaminophenylacetic acid and derivatives, such as celecoxib (CELEBREX™), rofecoxib (VIOXX™), etoricoxib, valdecoxib or a 5-alkyl-2-arylaminophenylacetic acid, such as 5-methyl-2-(2′-chloro-6′-fluoroanilino)phenyl acetic acid, lumiracoxib.

The term “bisphosphonates” as used herein includes, but is not limited to, etridonic, clodronic, tiludronic, pamidronic, alendronic, ibandronic, risedronic and zoledronic acid. Etridonic acid is marketed under the trade name DIDRONEL™. Clodronic acid is marketed under the trade name BONEFOS™ Tiludronic acid is marketed under the trade name Skelid™. Pamidronic acid is marketed under the trade name AREDIA™. Alendronic acid is marketed under the trade name FOSAMAX™. Ibandronic acid is marketed under the trade name BONDRANAT™. Risedronic acid is marketed under the trade name ACTONEL™. Zoledronic acid is marketed under the trade name ZOMETA™. The term “mTOR inhibitors” relates to compounds which inhibit the mammalian target of rapamycin (mTOR) and which possess antiproliferative activity such as sirolimus (RAPAMUNE®), everolimus (CERTICAN™), CCI-779 and ABT578.

The term “heparanase inhibitor” as used herein refers to compounds which target, decrease or inhibit heparin sulfate degradation. The term includes, but is not limited to, PI-88. The term “biological response modifier” as used herein refers to a lymphokine or interferons.

The term “inhibitor of Ras oncogenic isoforms”, such as H-Ras, K-Ras, or N-Ras, as used herein refers to compounds which target, decrease or inhibit the oncogenic activity of Ras; for example, a “farnesyl transferase inhibitor” such as L-744832, DK8G557 or R115777 (ZARNESTRA™). The term “telomerase inhibitor” as used herein refers to compounds which target, decrease or inhibit the activity of telomerase. Compounds which target, decrease or inhibit the activity of telomerase are especially compounds which inhibit the telomerase receptor, such as telomestatin.

The term “methionine aminopeptidase inhibitor” as used herein refers to compounds which target, decrease or inhibit the activity of methionine aminopeptidase. Compounds which target, decrease or inhibit the activity of methionine aminopeptidase include, but are not limited to, bengamide or a derivative thereof.

The term “proteasome inhibitor” as used herein refers to compounds which target, decrease or inhibit the activity of the proteasome. Compounds which target, decrease or inhibit the activity of the proteasome include, but are not limited to, Bortezomib (VELCADE™) and MLN 341.

The term “matrix metalloproteinase inhibitor” or (“MMP” inhibitor) as used herein includes, but is not limited to, collagen peptidomimetic and nonpeptidomimetic inhibitors, tetracycline derivatives, e.g., hydroxamate peptidomimetic inhibitor batimastat and its orally bioavailable analogue marimastat (BB-2516), prinomastat (AG3340), metastat (NSC 683551) BMS-279251, BAY 12-9566, TAA211, MMI270B or AAJ996.

The term “compounds used in the treatment of hematologic malignancies” as used herein includes, but is not limited to, FMS-like tyrosine kinase inhibitors, which are compounds targeting, decreasing or inhibiting the activity of FMS-like tyrosine kinase receptors (Flt-3R); interferon, 1-β-D-arabinofuransylcytosine (ara-c) and bisulfan; and ALK inhibitors, which are compounds which target, decrease or inhibit anaplastic lymphoma kinase.

Compounds which target, decrease or inhibit the activity of FMS-like tyrosine kinase receptors (Flt-3R) are especially compounds, proteins or antibodies which inhibit members of the Flt-3R receptor kinase family, such as PKC412, midostaurin, a staurosporine derivative, SU11248 and MLN518.

The term “HSP90 inhibitors” as used herein includes, but is not limited to, compounds targeting, decreasing or inhibiting the intrinsic ATPase activity of HSP90; degrading, targeting, decreasing or inhibiting the HSP90 client proteins via the ubiquitin proteosome pathway. Compounds targeting, decreasing or inhibiting the intrinsic ATPase activity of HSP90 are especially compounds, proteins or antibodies which inhibit the ATPase activity of HSP90, such as 17-allylamino, 17-demethoxygeldanamycin (17AAG), a geldanamycin derivative; other geldanamycin related compounds; radicicol and HDAC inhibitors.

The term “antiproliferative antibodies” as used herein includes, but is not limited to, trastuzumab (HERCEPTIN™), Trastuzumab-DM1, erbitux, bevacizumab (AVASTIN™), rituximab (RITUXAN®), PR064553 (anti-CD40) and 2C4 Antibody. By antibodies is meant intact monoclonal antibodies, polyclonal antibodies, multispecific antibodies formed from at least 2 intact antibodies, and antibodies fragments so long as they exhibit the desired biological activity.

For the treatment of acute myeloid leukemia (AML), compounds of the current invention can be used in combination with standard leukemia therapies, especially in combination with therapies used for the treatment of AML. In particular, compounds of the current invention can be administered in combination with, for example, farnesyl transferase inhibitors and/or other drugs useful for the treatment of AML, such as Daunorubicin, Adriamycin, Ara-C, VP-16, Teniposide, Mitoxantrone, Idarubicin, Carboplatinum and PKC412.

Other anti-leukemic compounds include, for example, Ara-C, a pyrimidine analog, which is the 2′-alpha-hydroxy ribose (arabinoside) derivative of deoxycytidine. Also included is the purine analog of hypoxanthine, 6-mercaptopurine (6-MP) and fludarabine phosphate. Compounds which target, decrease or inhibit activity of histone deacetylase (HDAC) inhibitors such as sodium butyrate and suberoylanilide hydroxamic acid (SAHA) inhibit the activity of the enzymes known as histone deacetylases. Specific HDAC inhibitors include MS275, SAHA, FK228 (formerly FR901228), Trichostatin A and compounds disclosed in U.S. Pat. No. 6,552,065 including, but not limited to, N-hydroxy-3-[4-[[[2-(2-methyl-1H-indol-3-yl)-ethyl]-amino]methyl]phenyl]-2E-2-propenamide, or a pharmaceutically acceptable salt thereof and N-hydroxy-3-[4-[(2-hydroxyethyl){2-(1H-indol-3-yl)ethyl]-amino]methyl]phenyl]-2E-2-propenamide, or a pharmaceutically acceptable salt thereof, especially the lactate salt. Somatostatin receptor antagonists as used herein refer to compounds which target, treat or inhibit the somatostatin receptor such as octreotide, and SOM230. Tumor cell damaging approaches refer to approaches such as ionizing radiation. The term “ionizing radiation” referred to above and hereinafter means ionizing radiation that occurs as either electromagnetic rays (such as X-rays and gamma rays) or particles (such as alpha and beta particles). Ionizing radiation is provided in, but not limited to, radiation therapy and is known in the art. See Hellman, Principles of Radiation Therapy, Cancer, in Principles and Practice of Oncology, Devita et al., Eds., 4^{th} Edition, Vol. 1, pp. 248-275 (1993).

Also included are EDG binders and ribonucleotide reductase inhibitors. The term “EDG binders” as used herein refers to a class of immunosuppressants that modulates lymphocyte recirculation, such as FTY720. The term “ribonucleotide reductase inhibitors” refers to pyrimidine or purine nucleoside analogs including, but not limited to, fludarabine and/or cytosine arabinoside (ara-C), 6-thioguanine, 5-fluorouracil, cladribine, 6-mercaptopurine (especially in combination with ara-C against ALL) and/or pentostatin. Ribonucleotide reductase inhibitors are especially hydroxyurea or 2-hydroxy-1H-isoindole-1,3-dione derivatives.

Also included are in particular those compounds, proteins or monoclonal antibodies of VEGF such as 1-(4-chloroanilino)-4-(4-pyridylmethyl)phthalazine or a pharmaceutically acceptable salt thereof, 1-(4-chloroanilino)-4-(4-pyridylmethyl)phthalazine succinate; ANGIOSTATIN™; ENDOSTATIN™; anthranilic acid amides; ZD4190; Zd₆474; SU5416; SU6668; bevacizumab; or anti-VEGF antibodies or anti-VEGF receptor antibodies, such as rhuMAb and RHUFab, VEGF aptamer such as Macugon; FLT-4 inhibitors, FLT-3 inhibitors, VEGFR-2 IgGI antibody, Angiozyme (RPI 4610) and Bevacizumab (AVASTIN™).

Photodynamic therapy as used herein refers to therapy which uses certain chemicals known as photosensitizing compounds to treat or prevent cancers. Examples of photodynamic therapy include treatment with compounds, such as VISUDYNE™ and porfimer sodium.

Angiostatic steroids as used herein refers to compounds which block or inhibit angiogenesis, such as, e.g., anecortave, triamcinolone, hydrocortisone, 11-α-epihydrocotisol, cortexolone, 17α-hydroxyprogesterone, corticosterone, desoxycorticosterone, testosterone, estrone and dexamethasone.

Implants containing corticosteroids refers to compounds, such as fluocinolone and dexamethasone.

Other chemotherapeutic compounds include, but are not limited to, plant alkaloids, hormonal compounds and antagonists; biological response modifiers, preferably lymphokines or interferons; antisense oligonucleotides or oligonucleotide derivatives; shRNA or siRNA; or miscellaneous compounds or compounds with other or unknown mechanism of action.

The structure of the active compounds identified by code numbers, generic or trade names may be taken from the actual edition of the standard compendium “The Merck Index” or from databases, e.g., Patents International (e.g., IMS World Publications).

### Exemplary Immune Checkpoint Inhibitors

In some embodiments, an immuno-oncology agent is an immune checkpoint inhibitor as described herein.

The term “checkpoint inhibitor” as used herein relates to agents useful in preventing cancer cells from avoiding the immune system of the patient. One of the major mechanisms of anti-tumor immunity subversion is known as “T-cell exhaustion,” which results from chronic exposure to antigens that has led to up-regulation of inhibitory receptors. These inhibitory receptors serve as immune checkpoints in order to prevent uncontrolled immune reactions.

PD-1 and co-inhibitory receptors such as cytotoxic T-lymphocyte antigen 4 (CTLA-4, B and T Lymphocyte Attenuator (BTLA; CD272), T cell Immunoglobulin and Mucin domain-3 (Tim-3), Lymphocyte Activation Gene-3 (Lag-3; CD223), and others are often referred to as a checkpoint regulators. They act as molecular “gatekeepers” that allow extracellular information to dictate whether cell cycle progression and other intracellular signaling processes should proceed.

In some embodiments, an immune checkpoint inhibitor is an antibody to PD-1. PD-1 binds to the programmed cell death 1 receptor (PD-1) to prevent the receptor from binding to the inhibitory ligand PDL-1, thus overriding the ability of tumors to suppress the host anti-tumor immune response.

In some embodiments, the checkpoint inhibitor is a biologic therapeutic or a small molecule. In some embodiments, the checkpoint inhibitor is a monoclonal antibody, a humanized antibody, a fully human antibody, a fusion protein or a combination thereof. In some embodiments, the checkpoint inhibitor inhibits a checkpoint protein selected from CTLA-4, PDL1, PDL2, PD1, B7-H3, B7-H4, BTLA, HVEM, TIM3, GAL9, LAG3, VISTA, KIR, 2B4, CD160, CGEN-15049, CHK 1, CHK2, A2aR, B-7 family ligands or a combination thereof. In some embodiments, the checkpoint inhibitor interacts with a ligand of a checkpoint protein selected from CTLA-4, PDL1, PDL2, PD1, B7-H3, B7-H4, BTLA, HVEM, TIM3, GAL9, LAG3, VISTA, KIR, 2B4, CD160, CGEN-15049, CHK 1, CHK2, A2aR, B-7 family ligands or a combination thereof. In some embodiments, the checkpoint inhibitor is an immunostimulatory agent, a T cell growth factor, an interleukin, an antibody, a vaccine or a combination thereof. In some embodiments, the interleukin is IL-7 or IL-15. In some embodiments, the interleukin is glycosylated IL-7. In an additional aspect, the vaccine is a dendritic cell (DC) vaccine.

Checkpoint inhibitors include any agent that blocks or inhibits in a statistically significant manner, the inhibitory pathways of the immune system. Such inhibitors can include small molecule inhibitors or can include antibodies, or antigen binding fragments thereof, that bind to and block or inhibit immune checkpoint receptors or antibodies that bind to and block or inhibit immune checkpoint receptor ligands. Illustrative checkpoint molecules that can be targeted for blocking or inhibition include, but are not limited to, CTLA-4, PDL1, PDL2, PD1, B7-H3, B7-H4, BTLA, HVEM, GAL9, LAG3, TIM3, VISTA, KIR, 2B4 (belongs to the CD2 family of molecules and is expressed on all NK, γδ, and memory CD8⁺ (αβ) T cells), CD160 (also referred to as BY55), CGEN-15049, CHK 1 and CHK2 kinases, A2aR, and various B-7 family ligands. B7 family ligands include, but are not limited to, B7-1, B7-2, B7-DC, B7-H1, B7-H2, B7-H3, B7-H4, B7-H5, B7-H6 and B7-H7. Checkpoint inhibitors include antibodies, or antigen binding fragments thereof, other binding proteins, biologic therapeutics, or small molecules, that bind to and block or inhibit the activity of one or more of CTLA-4, PDL1, PDL2, PD1, BTLA, HVEM, TIM3, GAL9, LAG3, VISTA, KIR, 2B4, CD 160 and CGEN-15049. Illustrative immune checkpoint inhibitors include, but are not limited to, Tremelimumab (CTLA-4 blocking antibody), anti-OX40, PD-L1 monoclonal Antibody (Anti-B7-H1; MEDI4736), MK-3475 (PD-1 blocker), Nivolumab (anti-PD1 antibody), CT-011 (anti-PD1 antibody), BY55 monoclonal antibody, AMP224 (anti-PDL1 antibody), BMS-936559 (anti-PDL1 antibody), MPLDL3280A (anti-PDL1 antibody), MSB0010718C (anti-PDL1 antibody), and ipilimumab (anti-CTLA-4 checkpoint inhibitor). Checkpoint protein ligands include, but are not limited to PD-L1, PD-L2, B7-H3, B7-H4, CD28, CD86 and TIM-3.

In certain embodiments, the immune checkpoint inhibitor is selected from a PD-1 antagonist, a PD-L1 antagonist, and a CTLA-4 antagonist. In some embodiments, the checkpoint inhibitor is selected from the group consisting of nivolumab (OPDIVO®), ipilimumab (YERVOY®), and pembrolizumab (KEYTRUDA®). In some embodiments, the checkpoint inhibitor is selected from nivolumab (anti-PD-1 antibody, OPDIVO@, Bristol-Myers Squibb); pembrolizumab (anti-PD-1 antibody, KEYTRUDA®, Merck); ipilimumab (anti-CTLA-4 antibody, YERVOY®, Bristol-Myers Squibb); durvalumab (anti-PD-L1 antibody, IMFINZI®, AstraZeneca); and atezolizumab (anti-PD-L1 antibody, TECENTRIQ®, Genentech).

In some embodiments, the checkpoint inhibitor is selected from the group consisting of lambrolizumab (MK-3475), nivolumab (BMS-936558), pidilizumab (CT-011), AMP-224, MDX-1105, MED14736, MPDL3280A, BMS-936559, ipilimumab, lirlumab, IPH2101, pembrolizumab (KEYTRUDA®), and tremelimumab.

In some embodiments, an immune checkpoint inhibitor is REGN2810 (Regeneron), an anti-PD-1 antibody tested in patients with basal cell carcinoma (NCT03132636); NSCLC (NCT03088540); cutaneous squamous cell carcinoma (NCT02760498); lymphoma (NCT02651662); and melanoma (NCT03002376); pidilizumab (CureTech), also known as CT-011, an antibody that binds to PD-1, in clinical trials for diffuse large B-cell lymphoma and multiple myeloma; avelumab (BAVENCIO®, Pfizer/Merck KGaA), also known as MSB0010718C), a fully human IgGI anti-PD-L1 antibody, in clinical trials for non-small cell lung cancer, Merkel cell carcinoma, mesothelioma, solid tumors, renal cancer, ovarian cancer, bladder cancer, head and neck cancer, and gastric cancer; or PDR001 (Novartis), an inhibitory antibody that binds to PD-1, in clinical trials for non-small cell lung cancer, melanoma, triple negative breast cancer and advanced or metastatic solid tumors. Tremelimumab (CP-675,206; Astrazeneca) is a fully human monoclonal antibody against CTLA-4 that has been in studied in clinical trials for a number of indications, including: mesothelioma, colorectal cancer, kidney cancer, breast cancer, lung cancer and non-small cell lung cancer, pancreatic ductal adenocarcinoma, pancreatic cancer, germ cell cancer, squamous cell cancer of the head and neck, hepatocellular carcinoma, prostate cancer, endometrial cancer, metastatic cancer in the liver, liver cancer, large B-cell lymphoma, ovarian cancer, cervical cancer, metastatic anaplastic thyroid cancer, urothelial cancer, fallopian tube cancer, multiple myeloma, bladder cancer, soft tissue sarcoma, and melanoma. AGEN-1884 (Agenus) is an anti-CTLA4 antibody that is being studied in Phase 1 clinical trials for advanced solid tumors (NCT02694822).

In some embodiments, a checkpoint inhibitor is an inhibitor of T-cell immunoglobulin mucin containing protein-3 (TIM-3). TIM-3 inhibitors that may be used in the present invention include TSR-022, LY3321367 and MBG453. TSR-022 (Tesaro) is an anti-TIM-3 antibody which is being studied in solid tumors (NCT02817633). LY3321367 (Eli Lilly) is an anti-TIM-3 antibody which is being studied in solid tumors (NCT03099109). MBG453 (Novartis) is an anti-TIM-3 antibody which is being studied in advanced malignancies (NCT02608268).

In some embodiments, a checkpoint inhibitor is an inhibitor of T cell immunoreceptor with Ig and ITIM domains, or TIGIT, an immune receptor on certain T cells and NK cells. TIGIT inhibitors that may be used in the present invention include BMS-986207 (Bristol-Myers Squibb), an anti-TIGIT monoclonal antibody (NCT02913313); OMP-313M32 (Oncomed); and anti-TIGIT monoclonal antibody (NCT03119428).

In some embodiments, a checkpoint inhibitor is an inhibitor of Lymphocyte Activation Gene-3 (LAG-3). LAG-3 inhibitors that may be used in the present invention include BMS-986016 and REGN3767 and IMP321. BMS-986016 (Bristol-Myers Squibb), an anti-LAG-3 antibody, is being studied in glioblastoma and gliosarcoma (NCT02658981). REGN3767 (Regeneron), is also an anti-LAG-3 antibody, and is being studied in malignancies (NCT03005782). IMP321 (Immutep S.A.) is an LAG-3-Ig fusion protein, being studied in melanoma (NCT02676869); adenocarcinoma (NCT02614833); and metastatic breast cancer (NCT00349934).

Checkpoint inhibitors that can be used in the present invention include OX40 agonists. OX40 agonists that are being studied in clinical trials include PF-04518600/PF-8600 (Pfizer), an agonistic anti-OX40 antibody, in metastatic kidney cancer (NCT03092856) and advanced cancers and neoplasms (NCT02554812; NCT05082566); GSK3174998 (Merck), an agonistic anti-OX40 antibody, in Phase 1 cancer trials (NCT02528357); MEDI0562 (Medimmune/AstraZeneca), an agonistic anti-OX40 antibody, in advanced solid tumors (NCT02318394 and NCT02705482); MED16469, an agonistic anti-OX40 antibody (Medimmune/AstraZeneca), in patients with colorectal cancer (NCT02559024), breast cancer (NCT01862900), head and neck cancer (NCT02274155) and metastatic prostate cancer (NCT01303705); and BMS-986178 (Bristol-Myers Squibb) an agonistic anti-OX40 antibody, in advanced cancers (NCT02737475).

Checkpoint inhibitors that can be used in the present invention include CD137 (also called 4-1BB) agonists. CD 137 agonists that are being studied in clinical trials include utomilumab (PF-05082566, Pfizer) an agonistic anti-CD137 antibody, in diffuse large B-cell lymphoma (NCT02951156) and in advanced cancers and neoplasms (NCT02554812 and NCT05082566); urelumab (BMS-663513, Bristol-Myers Squibb), an agonistic anti-CD137 antibody, in melanoma and skin cancer (NCT02652455) and glioblastoma and gliosarcoma (NCT02658981); and CTX-471 (Compass Therapeutics), an agonistic anti-CD137 antibody in metastatic or locally advanced malignancies (NCT03881488).

Checkpoint inhibitors that can be used in the present invention include CD27 agonists. CD27 agonists that are being studied in clinical trials include varlilumab (CDX-1127, Celldex Therapeutics) an agonistic anti-CD27 antibody, in squamous cell head and neck cancer, ovarian carcinoma, colorectal cancer, renal cell cancer, and glioblastoma (NCT02335918); lymphomas (NCT01460134); and glioma and astrocytoma (NCT02924038).

Checkpoint inhibitors that can be used in the present invention include glucocorticoid-induced tumor necrosis factor receptor (GITR) agonists. GITR agonists that are being studied in clinical trials include TRx518 (Leap Therapeutics), an agonistic anti-GITR antibody, in malignant melanoma and other malignant solid tumors (NCT01239134 and NCT02628574); GWN323 (Novartis), an agonistic anti-GITR antibody, in solid tumors and lymphoma (NCT 02740270); INCAGN01876 (Incyte/Agenus), an agonistic anti-GITR antibody, in advanced cancers (NCT02697591 and NCT03126110); MK-4166 (Merck), an agonistic anti-GITR antibody, in solid tumors (NCT02132754) and MEDI1873 (Medimmune/AstraZeneca), an agonistic hexameric GITR-ligand molecule with a human IgGI Fc domain, in advanced solid tumors (NCT02583165).

Checkpoint inhibitors that can be used in the present invention include inducible T-cell co-stimulator (ICOS, also known as CD278) agonists. ICOS agonists that are being studied in clinical trials include MEDI-570 (Medimmune), an agonistic anti-ICOS antibody, in lymphomas (NCT02520791); GSK3359609 (Merck), an agonistic anti-ICOS antibody, in Phase 1 (NCT02723955); JTX-2011 (Jounce Therapeutics), an agonistic anti-ICOS antibody, in Phase 1 (NCT02904226).

Checkpoint inhibitors that can be used in the present invention include killer IgG-like receptor (KIR) inhibitors. KIR inhibitors that are being studied in clinical trials include lirilumab (IPH2102/BMS-986015, Innate Pharma/Bristol-Myers Squibb), an anti-KIR antibody, in leukemias (NCT01687387, NCT02399917, NCT02481297, NCT02599649), multiple myeloma (NCT02252263), and lymphoma (NCT01592370); IPH2101 (1-7F9, Innate Pharma) in myeloma (NCT01222286 and NCT01217203); and IPH4102 (Innate Pharma), an anti-KIR antibody that binds to three domains of the long cytoplasmic tail (KIR3DL2), in lymphoma (NCT02593045).

Checkpoint inhibitors that can be used in the present invention include CD47 inhibitors of interaction between CD47 and signal regulatory protein alpha (SIRPa). CD47/SIRPa inhibitors that are being studied in clinical trials include ALX-148 (Alexo Therapeutics), an antagonistic variant of (SIRPa) that binds to CD47 and prevents CD47/SIRPa-mediated signaling, in phase 1 (NCT03013218); TTI-621 (SIRPa-Fc, Trillium Therapeutics), a soluble recombinant fusion protein created by linking the N-terminal CD47-binding domain of SIRPa with the Fc domain of human IgGI, acts by binding human CD47, and preventing it from delivering its “do not eat” signal to macrophages, is in clinical trials in Phase 1 (NCT02890368 and NCT02663518); CC-90002 (Celgene), an anti-CD47 antibody, in leukemias (NCT02641002); and Hu5F9-G4 (Forty Seven, Inc.), in colorectal neoplasms and solid tumors (NCT02953782), acute myeloid leukemia (NCT02678338) and lymphoma (NCT02953509).

Checkpoint inhibitors that can be used in the present invention include CD73 inhibitors. CD73 inhibitors that are being studied in clinical trials include MEDI9447 (Medimmune), an anti-CD73 antibody, in solidtumors (NCT02503774); and BMS-986179 (Bristol-Myers Squibb), an anti-CD73 antibody, in solid tumors (NCT02754141).

Checkpoint inhibitors that can be used in the present invention include agonists of stimulator of interferon genes protein (STING, also known as transmembrane protein 173, or TMEM173). Agonists of STING that are being studied in clinical trials include MK-1454 (Merck), an agonistic synthetic cyclic dinucleotide, in lymphoma (NCT03010176); and ADU-S100 (MIW815, Aduro Biotech/Novartis), an agonistic synthetic cyclic dinucleotide, in Phase 1 (NCT02675439 and NCT03172936).

Checkpoint inhibitors that can be used in the present invention include CSF1R inhibitors. CSF1R inhibitors that are being studied in clinical trials include pexidartinib (PLX3397, Plexxikon), a CSF 1R small molecule inhibitor, in colorectal cancer, pancreatic cancer, metastatic and advanced cancers (NCT02777710) and melanoma, non-small cell lung cancer, squamous cell head and neck cancer, gastrointestinal stromal tumor (GIST) and ovarian cancer (NCT02452424); and IMC-CS4 (LY3022855, Lilly), an anti-CSF-1R antibody, in pancreatic cancer (NCT03153410), melanoma (NCT03101254), and solid tumors (NCT02718911); and BLZ945 (4-[2((1R,2R)-2-hydroxycyclohexylamino)-benzothiazol-6-yloxyl]-pyridine-2-carboxylic acid methylamide, Novartis), an orally available inhibitor of CSF1R, in advanced solid tumors (NCT02829723).

Checkpoint inhibitors that can be used in the present invention include NKG2A receptor inhibitors. NKG2A receptor inhibitors that are being studied in clinical trials include monalizumab (IPH2201, Innate Pharma), an anti-NKG2A antibody, in head and neck neoplasms (NCT02643550) and chronic lymphocytic leukemia (NCT02557516).

In some embodiments, the immune checkpoint inhibitor is selected from nivolumab, pembrolizumab, ipilimumab, avelumab, durvalumab, atezolizumab, or pidilizumab.

### EXEMPLIFICATION

### General Synthetic Methods

The following examples are intended to illustrate the invention and are not to be construed as being limitations thereon. Temperatures are given in degrees centigrade. If not mentioned otherwise, all evaporations are performed under reduced pressure, preferably between about 15 mm Hg and 100 mm Hg (=20-133 mbar). The structure of final products, intermediates and starting materials is confirmed by standard analytical methods, e.g., microanalysis and spectroscopic characteristics, e.g., MS, IR, NMR. Abbreviations used are those conventional in the art.

All starting materials, building blocks, reagents, acids, bases, dehydrating agents, solvents, and catalysts utilized to synthesis the compounds of the present invention are either commercially available or can be produced by organic synthesis methods known to one of ordinary skill in the art (Houben-Weyl 4th Ed. 1952, Methods of Organic Synthesis, Thieme, Volume 21). Further, the compounds of the present invention can be produced by organic synthesis methods known to one of ordinary skill in the art as shown in the following examples.

All reactions are carried out under nitrogen or argon unless otherwise stated.

Proton NMR (¹H NMR) is conducted in deuterated solvent. In certain compounds disclosed herein, one or more ¹H shifts overlap with residual proteo solvent signals; these signals have not been reported in the experimental provided hereinafter.

As depicted in the Examples below, in certain exemplary embodiments, compounds are prepared according to the following general procedures. It will be appreciated that, although the general methods depict the synthesis of certain compounds of the present invention, the following general methods, and other methods known to one of ordinary skill in the art, can be applied to all compounds and subclasses and species of each of these compounds, as described herein.

### Abbreviations

Ac: acetyl
ACN: acetonitrile
AcOH: acetic acid
BINAP: (2,2′-bis(diphenylphosphino)-1,1′-binaphthyl)
BnBr: Benzyl bromide
Boc: tert-butoxycarbonyl
Boc2O: di-tert-butyl dicarbonate
Cbz: benzyloxycarbonyl
COD: 1,5-Cyclooctadiene
DCE: 1,2-dichloroethane
DCM: dichloromethane
dcpp: 1,3-Bis(dicyclohexylphosphino) propane bis(tetrafluoroborate)
DDQ: 2,3-Dichloro-5,6-dicyano-1,4-benzoquinone
DIAD: 1,3-bis(dicyclohexylphosphino)propane bis(tetrafluoroborate)
DIPEA or DIEA: N,N-diisopropylethylamine
DMAP: 4-dimethylaminopyridine
DMF: N,N-dimethylformamide
DMSO: dimethyl sulfoxide
EA: ethyl acetate
EDC or EDCI: 1-(3-dimethylaminopropyl)-3-ethylcarbodiimide hydrochloride
ee: enantiomeric excess
eq: equivalents
Et3N: triethyl amine
EtI: ethyl iodide
EtOAc: ethyl acetate
EtOH: ethanol
FA: formic acid
Fmoc: fluorenylmethoxycarbonyl
FmocCl or Fmoc-Cl: 9-fluorenylmethoxycarbonyl chloride
h or hrs: hours
HATU: N,N,N′,N′-tetramethyl-O-(7-azabenzotriazol-1-yl)uronium hexafluorophosphate
HBTU: (2-(1H-benzotriazol-1-yl)-1,1,3,3-tetramethyluronium hexafluorophosphate,
hexafluorophosphate benzotriazole tetramethyl uronium)
HMPA: Hexamethylphosphoramide
HOAc: acetic acid
HOAt: 1-Hydroxy-7-azabenzotriazole
HOBt: Hydroxybenzotriazole
HPLC: high performance liquid chromatography
hr, hrs, or h: hour
IPA or i-PrOH: isopropyl alcohol
M: molar
M: molarity
MeCN: acetonitrile
MeOH: methanol
Mhz megahertz
min: minutes
mL: milliliters
mm: milimeters
mM: millμmolar
mmol: millμmoles
MS: mass spectrometry or mass spectrometer
MS: Molecule sieves
MTBE: Methyl tert-butyl ether
NaOBH(OAc)3: Sodium triacetoxyborohydride
NMR: Nuclear Magnetic Resonance
Pd(dppf)Cl₂: [1,1′-Bis(diphenylphosphino)ferrocene]dichloropalladium(II)
PE: petroleum ether
psi: pounds per square inch
Rf: retention factor
Rt: retention time
sat: saturated
SFC: supercritical fluid chromatography
t-BuONa: Sodium tert-butoxide
TEA: triethylamine
Tf: trifluoromethanesulfonyl
TFA: trifluoracetic acid
TFE: 2,2,2-trifluoroethanol
THF: tetrahydrofuran
TLC: thin layer chromatography
TMS: trimethyl silyl
TMSI: trimethylsilyl iodide
TMSOTf: Trimethylsilyl trifluoromethanesulfonate
μm: micrometers
pmol: micromoles
v/v: volume per volume

### General Conditions

Mass spectra were acquired on LC-MS systems using electrospray, chemical and electron impact ionization methods from a range of instruments of the following configurations: SHIMADZU LCMS-2020, Agilent 1200 LC/G1956A MSD and Agilent 1200\G6110A, Agilent 1200 LC & Agilent 6110 MSD Mass Spectrometer [M+H]⁺ refers to protonated molecular ion of the chemical species.

Chiral HPLC spectra were acquired on SFC systems (Agilent1260 & Berger) using Chiralpak AD-3, AS-3, OD-3, IF-3, IE-3, IA-3, IF-3, IK-3, AS-S & AD-S; Chiralcel OD-3, OD-S & OJ-S.

HPLC spectrum were detected at 254 nm and 220 nm, unless indicated otherwise.

NMR spectra were run on Bruker 400 MHz spectrometers using ICON-NMR, under TopSpin program control. Spectra were measured at 298K, unless indicated otherwise, and were referenced relative to the solvent resonance.

TABLE 2
LCMS Methods

| | | | Mobile | Mobile |
|---|---|---|---|---|
| Method | Gradient | Column | Phase A | Phase B |
| A | 0% B to 60% B | Kinetex | 0.0375% | 0.01875% |
| | over 0.6 min, hold | EVO C18 | TFA in | TFA in |
| | at 60% B for 0.18 | (2.1 × 30 mm, | water | ACN (v/v) |
| | min, 60% B to 0% | 5 μm) | (v/v) | |
| | B over 0.01 min, | | | |
| | hold 0% B for 0.01 | | | |
| | min; Flow rate: | | | |
| | 2.0 mL/min | | | |
| B | 0% B to 60% B | HALO C18 | 0.0375% | 0.01875% |
| | over 0.5 min, hold | (3.0 × 30 mm, | TFA in | TFA in |
| | at 60% B for 0.3 | 5.0 μm) | water | ACN (v/v) |
| | min, 60% B to 0% | | (v/v) | |
| | B over 0.01 min, | | | |
| | hold at 0% B for | | | |
| | 0.24 min; | | | |
| | Flow rate: | | | |
| | 1.5 mL/min | | | |
| C | 0% B to 60% B | Kinetex | 0.025% | ACN/ |
| | over 0.8 min, hold | EVO C18 | NH₃•H₂O in | |
| | at 60% B for 0.4 | C18 | water (v/v) | |
| | min, 60% B to 0% | (2.1 × 30 mm, | | |
| | B over 0.01 min, | 5 μm) | | |
| | hold at 0% B for | | | |
| | 0.34 min; | | | |
| | Flow rate: | | | |
| | 1.5 mL/min | | | |
| D | 5% B to 95% B | Kinetex | 0.0375% | 0.01875% |
| | over 0.6 min, hold | EVO C18 | TFA in | TFA in |
| | at 95% B for 0.18 | C18 | water | ACN (v/v) |
| | min, 95% B to 5% | (2.1 × 30 mm, | (v/v) | |
| | B over 0.01 min, | 5 μm) | | |
| | hold at 5% B for | | | |
| | 0.01 min; | | | |
| | Flow rate: | | | |
| | 2 mL/min | | | |
| E | 5% B to 95% B | HALO C18 | 0.0375% | 0.01875% |
| | over 0.5 min, hold | (3.0 × 30 mm, | TFA in | TFA in |
| | at 95% B for 0.3 | 5.0 μm) | water | ACN (v/v) |
| | min, 95% B to 5% | | (v/v) | |
| | B over 0.01 min, | | | |
| | hold at 5% B for | | | |
| | 0.24 min; | | | |
| | Flow rate: | | | |
| | 1.5 mL/min | | | |
| F | 5% B to 95% B | Gemini | 0.025% | ACN |
| | over 0.7 min, hold | 5 μm | NH₃•H₂O in | |
| | at 95% B for 0.2 | NX- | water (v/v) | |
| | min, 95% B to 5% | C18 110A | | |
| | B over 0.01 min, | (2*30 mm, | | |
| | hold at 5% B for | 5 μm) | | |
| | 0.09 min; | | | |
| | Flow rate: | | | |
| | 1.7 mL/min | | | |
| G | 5% B to 95% B | Kinetex | 0.025% | ACN |
| | over 0.8 min, hold | EVO C18 | NH₃•H₂O in | |
| | at 95% B for 0.4 | C18 | water (v/v) | |
| | min, 95% B to 5% | (2.1 × 30 mm, | | |
| | B over 0.01 min, | 5 μm) | | |
| | hold at 5% B for | | | |
| | 0.34 min; | | | |
| | Flow rate: | | | |
| | 1.5 mL/min | | | |
| J | 5% B to 40% B; | Chiralpak AD-3 | CO₂ | 0.05% DEA |
| | Flow rate: 3 | (50 × 4.6 mm, | | in MeOH |
| | mL/min | 3 μm) | | |
| K- | 5% B to 40% B; | Chiralpak AS-3 | CO₂ | 0.05% DEA |
| | Flow rate: 3 | (50 × 4.6 mm, | | in MeOH |
| | mL/min | 3 μm) | | |
| L | 40% B; Flow rate: | Chiralcel OD-3 | CO₂ | 0.05% DEA |
| | 3 mL/min | (50 × 4.6 mm, | | in EtOH/ |
| | | 3 μm) | | ACN |
| M | 50% B; Flow rate: | Chiralpak AS-3 | CO₂ | 0.05% DEA |
| | 3 mL/min | (50 × 4.6 mm, | | in MeOH/ |
| | | 3 μm) | | ACN |
| N | 60% B; Flow rate: | Chiralpak AD-3 | CO₂ | 0.05% DEA |
| | 3 mL/min | (50 × 4.6 mm, | | in MeOH/ |
| | | 3 μm) | | ACN |
| O | 100% B; Flow rate: | Chiralpak IF-3 | 0.05% IPA | 0.05% IPA |
| | 1 mL | (50 × 4.6 mm, | in hexane | in EtOH/ |
| | | 3 μm | | ACN |
| P | 60% B; Flow rate: | Chiralpak AS-3 | CO₂ | 0.05% IPA |
| | 3 mL/min | (50 × 4.6 mm, | | in ACN |
| | | 3 μm) | | |
| Q | 60% B; Flow rate: | Chiralpak | | 0.05% DEA |
| | 3 mL/min | (S,S)Whelk-0 1 | | in EtOH/ |
| | | | | ACN |
| R | 100% B; Flow rate: | Chiralpak IE-3 | 0.05% IPA | 0.05% IPA |
| | 1 mL/min | (50 × 4.6 mm, | in hexane | in EtOH/ |
| | | 3 μm) | | ACN |
| S | 40% B; Flow rate: | Chiralpak AS-3 | CO₂ | 0.05% DEA |
| | 3 mL/min | (50 × 4.6 mm, | | in MeOH/ |
| | | 3 μm) | | ACN |
| T | 80% B; Flow rate: | Chiralpak IA-3 | 0.05% IPA | 0.05% IPA |
| | 1 mL/min | (50 × 4.6 mm, | in hexane | in ACN |
| | | 3 μm) | | |
| U | 45% B; Flow rate: | Chiralcel OX | | 0.05% IPA |
| | 1 mL/min | | | in EtOH/ |
| | | | | ACN |
| V | 50% B; Flow rate: | Chiralpak IA-3 | 0.05% IPA | 0.05% IPA |
| | 1 mL/min | (50 × 4.6 mm, | in hexane | in EtOH/ |
| | | 3 μm) | | ACN |
| W | 40% B; Flow rate: | Chiralcel OD-3 | | 0.05% DEA |
| | 3 mL/min | (50 × 4.6 mm, | | in MeOH/ |
| | | 3 μm) | | ACN |
| X | 80% B; Flow rate: | Chiralpak IF-3 | 0.05% IPA | 0.05% IPA |
| | 1 mL/min | (5 × 4.6 mm, | in hexane | in EtOH/ |
| | | 3 μm | | ACN |
| Y | 50% B; Flow rate: | Chiralpak IA-3 | 0.05% IPA | 0.05% IPA |
| | 1 mL/min | (50 × 4.6 mm, | in hexane | in ACN |
| | | 3 μm) | | |
| Z | 40% B; Flow rate: | Chiralcel OJ-3 | | 0.05% DEA |
| | 3 mL/min | | | in MeOH |
| AA | 80% B; Flow rate: | Chiralpak IF-3 | | 0.05% IPA |
| | 1 mL/min | (50 × 4.6 mm, | | in ACN |
| | | 3 μm) | | |
| AB | 60% B; Flow rate: | Chiralpak IK-3 | CO₂ | 0.05% DEA |
| | 3 mL/min | (50 × 4.6 mm, | | in MeOH/ |
| | | 3 μm) | | ACN |
| AC | 80% B; Flow rate: | Chiralpak IA-3 | 0.05% IPA | 0.05% IPA |
| | 3 mL/min | (50 × 4.6 mm, | in hexane | in EtOH/ |
| | | 3 μm) | | ACN |

### Example 1: Synthesis of Compounds of the Invention

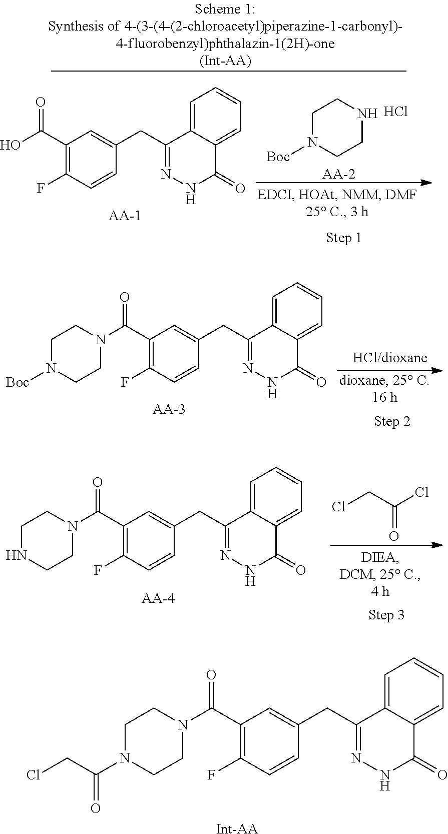

### Step 1: Synthesis of AA-3

To a mixture of AA-1 (5 g, 16.76 mmol, 1 eq), AA-2 (4.48 g, 20.12 mmol, 1.2 eq) and HOAt (2.28 g, 16.76 mmol, 1 eq) in DMF (50 mL) was added EDCI (9.64 g, 50.29 mmol, 3 eq) and NMM (8.48 g, 83.82 mmol, 9.22 mL, 5 eq). The mixture was stirred at 25° C. for 3 hrs. The reaction mixture was diluted with H₂O (100 mL) and filtered. Then filter cake was collected and concentrated under reduced pressure. AA-3 (7.5 g, 16.04 mmol, 95.67% yield, 99% purity) was obtained as a white solid. LCMS (Method E): Rt=0.509 min, [M−56+H]⁺=411.2. ¹H NMR (400 MHz, methanol-d₄) δ 8.37 (dd, J=1.2, 7.7 Hz, 1H), 7.97-7.92 (m, 1H), 7.89-7.80 (m, 2H), 7.50-7.44 (m, 1H), 7.36 (dd, J=2.2, 6.3 Hz, 1H), 7.15 (t, J=9.0 Hz, 1H), 4.38 (s, 2H), 3.76-3.69 (m, 2H), 3.51 (br s, 2H), 3.34 (br d, J=3.9 Hz, 2H), 3.27 (br s, 2H), 1.46 (s, 9H).

### Step 2: Synthesis of AA-4

To a solution of AA-3 (3.5 g, 7.50 mmol, 1 eq) in dioxane (20 mL) was added HCl/dioxane (4 M, 20 mL). The mixture was stirred at 25° C. for 16 h. The reaction mixture was concentrated under reduced pressure. AA-4 (3.9 g, crude) was obtained as a yellow oil. LCMS (Method E): Rt=0.364 min, [M+H]⁺=367.2.

### Step 3: Synthesis of Int-AA

To a solution of AA-4 (3.9 g, 10.64 mmol, 1 eq) in DCM (55 mL) was added DIEA (4.13 g, 31.93 mmol, 5.56 mL, 3 eq) and 2-chloroacetyl chloride (1.44 g, 12.77 mmol, 1.02 mL, 1.2 eq) at 0° C. Then the mixture was stirred at 25° C. for 4 hrs. The mixture was washed with brine (100 mL). The organic layer was dried over anhydrous Na₂SO₄, filtered and concentrated under reduced pressure. Int-AA (3.5 g, crude) was obtained as brown solid. LCMS (Method C): Rt=0.618 min, [M+H]⁺=443.3. ¹H NMR (400 MHz, chloroform-d) δ 11.23-10.87 (m, 1H), 8.48 (br d, J=6.1 Hz, 1H), 7.88-7.61 (m, 3H), 7.34 (br d, J=5.5 Hz, 2H), 7.05 (brt, J=7.9 Hz, 1H), 4.30 (s, 2H), 4.15-4.02 (m, 2H), 3.81 (s, 1H), 3.70 (s, 1H), 3.62 (br s, 2H), 3.54-3.45 (m, 2H), 3.44-3.29 (m, 2H).

### Alternate Step 3: Synthesis of Int-AA

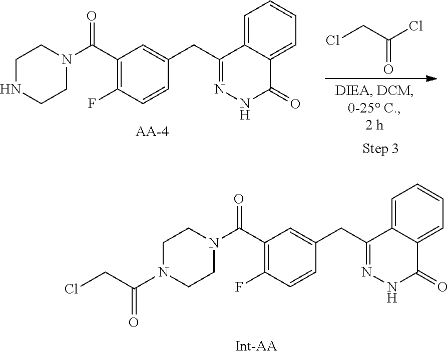

To a solution of AA-4 (1 g, 2.73 mmol, 1 eq) in DCM (14.5 mL) was added DIEA (1.06 g, 8.19 mmol, 1.43 mL, 3 eq) and 2-chloroacetyl chloride (369.91 mg, 3.28 mmol, 260.87 uL, 1.2 eq) at 0° C. Then the mixture was stirred at 25° C. for 2 hrs. The mixture was washed with brine (20 mL), dried over anhydrous Na₂SO₄, filtered and concentrated under reduced pressure to give a residue. Int-AA (900 mg, 1.78 mmol, 65.07% yield, 87.0% purity) was obtained as brown solid. LCMS (Method E): Rt=0.422 min, [M+H]⁺=443.3. ¹H NMR (400 MHz, chloroform-d) δ 10.93-10.72 (m, 1H), 8.55-8.41 (m, 1H), 7.87-7.65 (m, 3H), 7.34 (br d, J=5.8 Hz, 2H), 7.06 (brt, J=8.4 Hz, 1H), 4.30 (s, 2H), 4.17-4.01 (m, 3H), 3.90-3.83 (m, 1H), 3.81 (s, 1H), 3.62 (br d, J=4.3 Hz, 2H), 3.51 (br s, 1H), 3.47-3.25 (m, 2H).

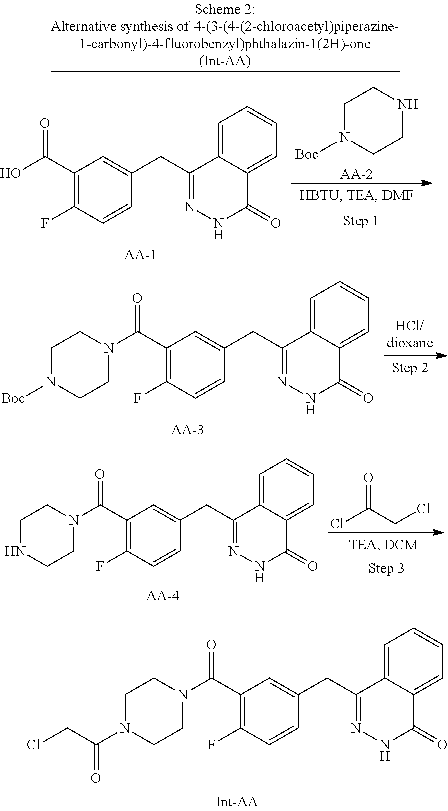

### Step 1: Synthesis of AA-3

To a solution of AA-2 (17 g, 91.27 mmol, 1.2 eq) and AA-1 (22.69 g, 76.06 mmol, 1 eq) in DMF (250 mL) was added HBTU (34.62 g, 91.27 mmol, 1.2 eq) and TEA (15.39 g, 152.12 mmol, 21.17 mL, 2 eq). The mixture was stirred at 25° C. for 2 hr. The crude product was triturated with EA (1000 mL). The solid was collected and concentrated under vacuum to give AA-3 (30 g, 63.54 mmol, 83.54% yield, 98.804% purity) as a white solid. LCMS (Method D): Rt=0.410 min, [M−55]⁺=411.2. ¹H NMR (400 MHz, DMSO-d₆) δ 12.58 (s, 1H), 8.25 (d, J=1.2 Hz, 1H), 7.95 (s, 1H), 7.91-7.80 (m, 2H), 7.45-7.40 (m, 1H), 7.36-7.31 (m, 1H), 7.25-7.20 (m, 1H), 4.32 (s, 2H), 3.59 (s, 2H), 3.41-3.38 (m, 2H), 3.24-3.19 (m, 2H), 3.17-3.09 (m, 2H), 1.40 (s, 9H). ¹⁹F NMR (376 MHz, DMSO-d₆) δ −119.77 ppm.

### Step 2: Synthesis of AA-4

To a solution of AA-3 (30 g, 64.31 mmol, 1 eq) in DCM (150 mL) was added HCl/dioxane (4 M, 150 mL, 9.33 eq). The mixture was stirred at 25° C. for 0.5 hr. The mixture was concentrated under vacuum. The crude product was used in the next step without purification. AA-4 (35 g, crude, HCl) was obtained as a white solid. LCMS (Method D): Rt=0.237 min, [M+H]⁺=367.2.

### Step 3: Synthesis of Int-AA

To AA-4 (5 g, 12.41 mmol, 1 eq, HCl) in DCM (40 mL) was added TEA (12.56 g, 124.12 mmol, 17.28 mL, 10 eq) at 25° C. and the mixture was slowly added to a stirring solution of 2-chloroacetyl chloride (19.54 g, 124.12 mmol, 10.34 mL, 10 eq) in DCM (40 mL) at 0° C. The resultant mixture was stirred at 25° C. for 12 min. The mixture was diluted with water (80 mL) and extracted with DCM (30 mL*3). The organic phase was concentrated to give a residue. The residue was purified by reversed phase HPLC (column: Welch Ultimate XB—SiOH 250 mm*70 mm*10 um; mobile phase: [EtOH+MeOH(4:1, neutral)]; gradient: 18%-43% B over 25 min) and concentrated under vacuum to give Int-AA (2 g, crude) as a yellow solid. LCMS (Method D): Rt=0.354 min, [M+H]⁺=443.0.

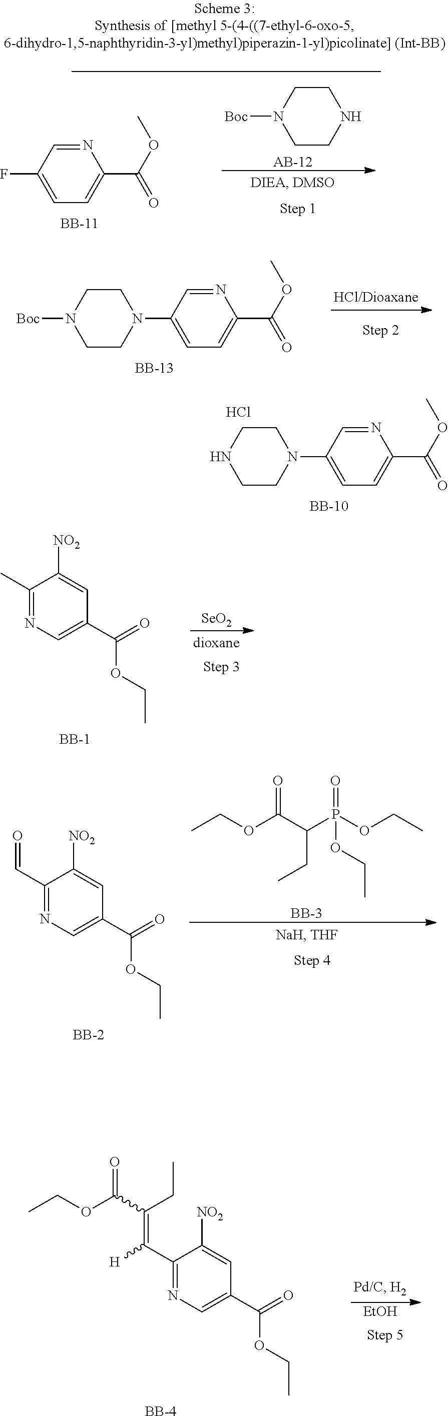

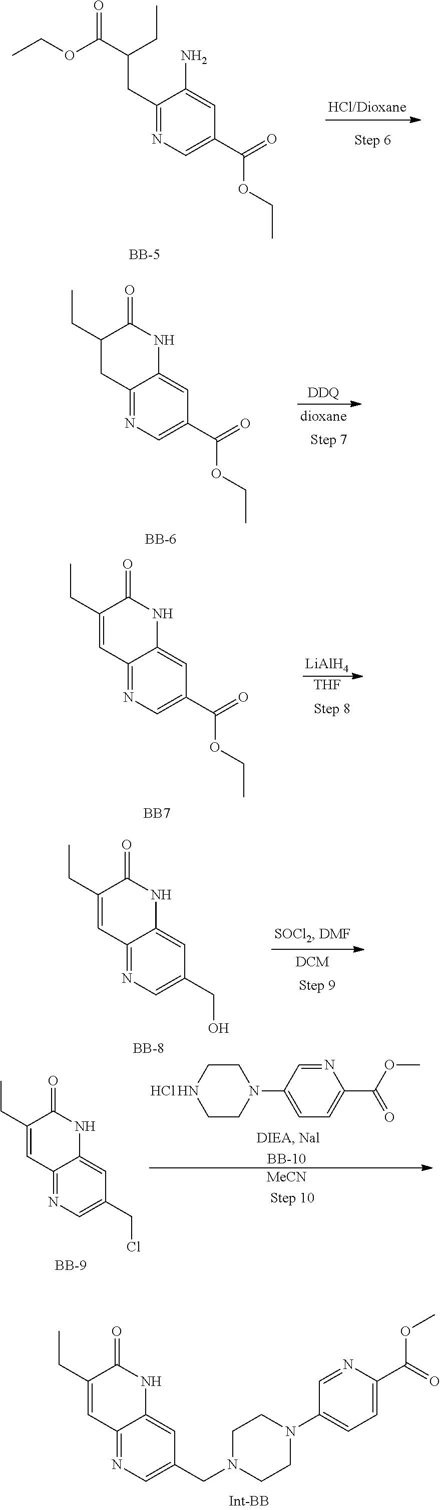

### Step 1: Synthesis of BB-13

To a solution of BB-11 (15 g, 96.70 mmol, 1 eq.) and BB-12 (21.54 g, 96.70 mmol, 1 eq.) in DMSO (150 mL) was added DIEA (24.99 g, 193.39 mmol, 33.69 mL, 2 eq.). The mixture was allowed to reach 100° C. and stirred for 16 hr. The reaction mixture was quenched with H₂O. The mixture was filtered under an inert atmosphere. The filter cake was washed with H₂O (100 mL*3) and was dissolved in EA. The solvent was dried over anhydrous Na₂SO₄, filtered and concentrated under reduced pressure to give the BB-13 (27.3 g, 84.95 mmol, 87.85% yield, 100% purity) as a yellow solid. LCMS (Method E): Rt=0.471 min, [M+H]⁺=322.1. ¹H NMR (400 MHz, CDCl₃) δ 8.31 (d, J=2.8 Hz, 1H), 7.98 (d, J=8.8 Hz, 1H), 7.23-7.07 (m, 1H), 3.93 (s, 3H), 3.63-3.55 (m, 4H), 3.37-3.27 (m, 4H), 1.46 (s, 9H).

### Step 2: Synthesis of BB-10

BB-13 (38.3 g, 119.18 mmol, 1 eq.) was dissolved in HCl/dioxane (380 mL). The mixture was then allowed to reach 25° C. and stirred for 2 hr. The reaction mixture was concentrated under reduced pressure to afford BB-10 (38 g, crude, HCl) as a light yellow solid which was used directly in the next step without further purification. LCMS (Method E): Rt=0.209 min, [M+H]⁺=222.2.

### Step 3: Synthesis of BB-2

To a solution of BB-1 (90 g, 428.19 mmol, 1 eq) in dioxane (250 mL) and H₂O (25 mL) was added SeO₂ (95.02 g, 856.38 mmol, 93.16 mL, 2 eq). The mixture was stirred at 100° C. for 20 hr. The reaction mixture was cooled to room temperature, filtered through a pad of celite and the celite was washed with ethyl acetate. The combined filtrate was concentrated to give crude product. The residue was purified by flash silica gel chromatography (440 g*2 SepaFlash silica flash column; eluent: 0-50% ethyl acetate/petroleum ether gradient at 150 mL/min). BB-2 (80 g, 239.10 mmol, 55.84% yield, 67% purity) was obtained as a red oil. LCMS (Method E): Rt=0.379 min, [M+H]⁺=225.0.

### Step 4: Synthesis of BB-4

To a stirred solution of NaH (15.85 g, 396.13 mmol, 60% purity, 2.4 eq) in anhydrous THF (380 mL) was added BB-3 (99.92 g, 396.13 mmol, 93.91 mL, 2.4 eq) dropwise with an addition funnel at 0° C. to give a grey colored mixture. The resulting mixture was stirred at 0° C. for 10 min and warmed to 40° C. and stirred at 40° C. for 5 min. The reaction mixture was cooled to −78° C. and to this cooled reaction mixture was then slowly added solution of BB-2 (37 g, 165.05 mmol, 1 eq) in THF (380 mL). The mixture was stirred at −78° C. for 15 min. The mixture was quenched with sat. NH₄Cl solution (1.5 L) and extracted with ethyl acetate (500 mL*3). The combined the organic layers were dried over sodium Na₂SO₄, filtered and concentrated to give crude product. The residue was purified by flash silica gel chromatography (330 g SepaFlash silica flash column*3, eluent: 0-20% ethyl acetate/petroleum ether gradient at 150 mL/min). BB-4 (91 g, crude) was obtained as yellow oil. LCMS (Method E): Rt=0.592, 0.621 min, [M+H]⁺=323.0.

### Step 5: Synthesis of BB-5

To a solution of BB-4 (43 g, 133.41 mmol, 1 eq) in EtOH (500 mL) was added Pd/C (10%, 7.1 g) under N₂ atmosphere. The suspension was degassed and purged with H₂ 3 times. The mixture was stirred under H₂ (20 psi) at 25° C. for 16 hr. The reaction mixture was filtered under inert atmosphere. The filter cake was washed with EtOH (100 L*3). The filtrate was concentrated under reduced pressure to afford BB-5 (51 g, crude) as a yellow oil which was used in the next step without further purification. LCMS (Method E): Rt=0.439 min, [M+H]⁺=295.2.

### Step 6: Synthesis of BB-6

To a solution of 1-5 (51 g, 173.27 mmol, 1 eq) was added HCl/dioxane (340 mL). The mixture was stirred at 25° C. for 1 hr. The reaction mixture was concentrated under reduced pressure. The pH was adjusted to 7 with aq. NaHCO₃, and the mixture was extracted with EA (1000 mL×4). The combined organic layers were dried over Na₂SO₄, filtered and concentrated under reduced pressure to give a residue. The crude product BB-6 (42 g, crude) was obtained as a yellow solid was used in the next step without further purification. LCMS: Rt=0.435 min, [M+H]⁺=249.1. ¹H NMR (400 MHz, DMSO-d₆) δ 10.30 (s, 1H), 8.60 (d, J=1.6 Hz, 1H), 7.66 (d, J=1.6 Hz, 1H), 4.42-4.25 (m, 2H), 3.23-3.14 (m, 1H), 2.98-2.88 (m, 1H), 2.66-2.52 (m, 1H), 1.79-1.66 (m, 1H), 1.51-1.39 (m, 1H), 1.32 (t, J=7.2 Hz, 3H), 0.93 (t, J=7.6 Hz, 3H).

### Step 7: Synthesis of BB-7

To a solution of BB-6 (42 g, 169.17 mmol, 1 eq) in dioxane (730 mL) was added DDQ (42.24 g, 186.08 mmol, 1.1 eq). The mixture was stirred at 110° C. for 3 hr. The solvent was removed under reduced pressure, sat. NaHCO₃ solution was added and the mixture stirred at 25° C. for 1 h. The solid was filtered off, washed with water, followed by MTBE. The resulting solid was dried under vacuum afford BB-7 (29 g, crude) as a brown. LCMS (Method (E): Rt=0.442 min, [M+H]⁺=247.1. ¹H NMR (400 MHz, DMSO-d₆) δ 12.04 (s, 1H), 8.89 (d, J=2.0 Hz, 1H), 8.15 (d, J=2.0 Hz, 1H), 7.82 (s, 1H), 4.38 (q, J=7.2 Hz, 2H), 2.61-2.53 (m, 2H), 1.35 (t, J=7.2 Hz, 3H), 1.19 (t, J=7.6 Hz, 3H).

### Step 8: Synthesis of BB-8

A solution of BB-7 (24 g, 97.46 mmol, 1 eq.) in THF (480 mL) was cooled to 0° C. via ice bath. The system was degassed and purged with nitrogen. Then, LiAlH₄ (2.5 M, 77.97 mL, 2 eq.) was introduced into the system drop wise. The mixture was maintained at 0° C. and stirred for 1.5 hr. The reaction mixture was quenched by addition of aqueous HCl (100 mL, 1M, added drop wise). The resulting mixture was concentrated under reduced pressure. The residue was diluted with water and then HCl (100 mL, 1M) was added to give a yellow suspension. The solid, which was filtered off and washed with water and MTBE was collected. The crude solid was suspended in a mixture of methanol and DCM (2:1, 1200 mL). The suspension was heated to reflux before the solid was filtered off and collected. The recrystallization process was repeated 5 times in order to remove most of the impurities and afford the target product. The filtrate was combined and concentrated to 300 mL. After the crystals were fully formed, the solid was filtered off, washed by MTBE and collected as another batch of target product. All batches were combined and concentrated under vacuum to afford BB-8 (19 g, 93.03 mmol, 95.46% yield) as an off-white solid. LCMS (Method E): Rt=0.308 min, [M+H]⁺=205.1. ¹H NMR (400 MHz, DMSO-d₆) δ 11.88 (br s, 1H), 8.37 (d, J=2.0 Hz, 1H), 7.73 (s, 1H), 7.63-7.59 (m, 1H), 5.46 (t, J=5.6 Hz, 1H), 4.61 (d, J=5.6 Hz, 2H), 2.57-2.51 (m, 2H), 1.18 (t, J=7.2 Hz, 3H).

### Step 9: Synthesis of BB-9

BB-8 (13 g, 63.66 mmol, 1 eq.) in DCM (260 mL) and DMF (6.37 mmol, 0.49 mL, 0.1 eq.) was cooled to 0° C. via ice bath. Then SOCl₂ (45.44 g, 381.93 mmol, 27.74 mL, 6 eq.) was added drop wise into the system. The mixture was then allowed to reach 25° C. and stirred for 6 hr. The reaction mixture was concentrated under reduced pressure to give BB-9 (13 g, crude) as a brown solid which was used directly into the next step without further purification. LCMS (Method E): Rt=0.417 min, [M+H]⁺=223.0.

### Step 10: Synthesis of Int-BB

To a solution of BB-9 (17.5 g, 78.59 mmol, 1 eq.) and BB-10 (17.39 g, 78.62 mmol, 1.00 eq.) in DMF (320 mL) was added DIPEA (30.33 g, 234.68 mmol, 40.88 mL, 3 eq) and KI (2.60 g, 15.65 mmol, 0.2 eq.) at 0° C. The mixture was then allowed to reach 50° C. and stirred for 2 hr. After the reaction was completed, the reaction mixture was diluted with a saturated sodium bicarbonate solution (300 mL), followed by acetonitrile (400 mL). The resulting mixture was stirred for 10 mins. The solid was filtered off and washed with H₂O (300 mL) and acetonitrile (300 mL) to give the crude product. The crude was dissolved with MeOH (1 L), recrystallized and filtered to yield Int-BB (20.5 g, 47.81 mmol, 61.12% yield, 95.024% purity) as an off-white. LCMS (Method E): Rt=0.358 min, [M+H]⁺=408.2. ¹H NMR (400 MHz, DMSO-d₆) δ 11.84 (br s, 1H), 8.44-8.30 (m, 2H), 7.87 (d, J=8.8 Hz, 1H), 7.75 (s, 1H), 7.62 (s, 1H), 7.33 (dd, J=2.6, 8.9 Hz, 1H), 3.80 (s, 3H), 3.65 (s, 2H), 3.39 (br s, 4H), 2.54 (br d, J=6.4 Hz, 6H), 1.18 (t, J=7.4 Hz, 3H).

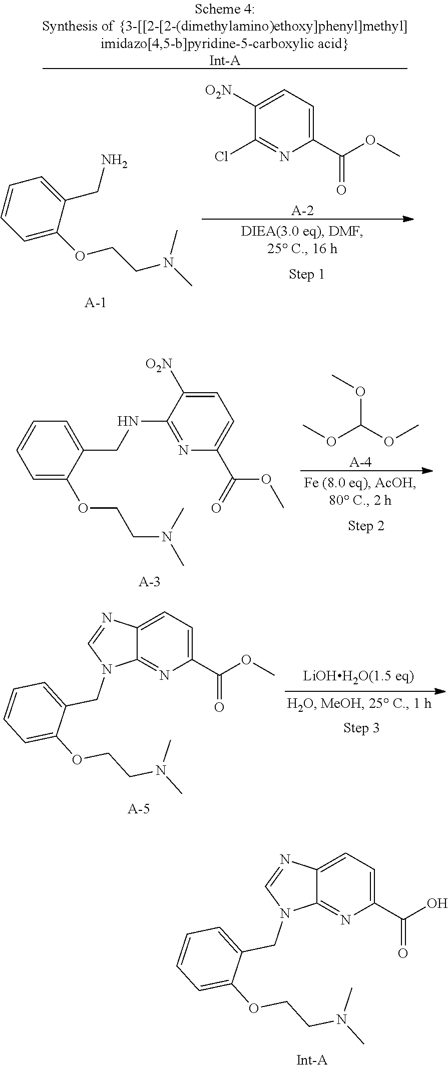

### Step 1: Synthesis of A-3

To A-1 (17 g, 87.51 mmol, 1 eq) was added DIEA (33.93 g, 262.52 mmol, 45.73 mL, 3 eq) in DMF (170 mL). Then A-2 (20.85 g, 96.26 mmol, 1.1 eq) was added. The reaction mixture was stirred at 25° C. for 16 hr. The mixture was quenched with water (500 mL) and aq. HCl (1 N) was added to adjust the pH to 2-3, and the mixture was extracted with EA (100 mL*2). To the aqueous phase was added NaHCO₃ to adjust the pH to 8-9, and it was then extracted with EA (100 mL*3), and the combined organic layers were dried over Na₂SO₄ and concentrated under reduced pressure to give A-3 (32 g, 81.20 mmol, 92.79% yield, 95% purity) as yellow solid. LCMS (Method E): Rt=0.434 min, [M+H]⁺=375.1. ¹H NMR (400 MHz, CDCl₃) δ 8.79-8.67 (m, 1H), 8.52 (d, J=8.4 Hz, 1H), 7.53 (d, J=6.8 Hz, 1H), 7.37 (d, J=8.4 Hz, 1H), 7.35-7.30 (m, 1H), 7.07-6.93 (m, 2H), 4.97 (br d, J=4.8 Hz, 2H), 4.69-4.56 (m, 2H), 4.03 (s, 3H), 3.66-3.48 (m, 2H), 3.00 (d, J=4.8 Hz, 6H).

### Step 2: Synthesis of A-5

To a solution of A-3 (32 g, 85.47 mmol, 1 eq) and A-4 (72.56 g, 683.78 mmol, 74.96 mL, 8 eq) in AcOH (300 mL) was added iron powder (38.19 g, 683.78 mmol, 8 eq). The mixture was stirred at 80° C. for 2 hr. The mixture was filtered through a pad of celite and the filtrate was adjusted with aq. Na₂CO₃ to pH=10, and then the mixture was extracted with DCM (100 mL*3). The combined organic layers were dried over Na₂SO₄, filtered and concentrated under reduced pressure to give A-5 (30 g, 77.03 mmol, 90.12% yield, 91% purity) as yellow solid. LCMS (Method E): Rt=0.392 min, [M+H]⁺=355.3. ¹H NMR (400 MHz, DMSO-d₆) δ 8.74 (s, 1H), 8.22 (d, J=8.4 Hz, 1H), 8.02 (d, J=8.4 Hz, 1H), 7.33-7.25 (m, 1H), 7.15-7.09 (m, 1H), 7.05 (d, J=8.4 Hz, 1H), 6.89 (m, 1H), 5.48 (s, 2H), 4.08 (m, 2H), 3.91 (s, 3H), 2.59 (m, 2H), 2.20 (s, 6H).

### Step 3: Synthesis of Int-A

To a solution of A-5 (30 g, 84.65 mmol, 1 eq) in MeOH (200 mL) and H₂O (50 mL) was added LiOH·H₂O (5.33 g, 126.97 mmol, 1.5 eq). The mixture was stirred at 25° C. for 1 hr. The mixture was concentrated in vacuum. The crude product was purified by reversed-phase HPLC (0.1% FA condition). After purification, the eluent was concentrated to remove organic solvents and then lyophilized to give Int-A (29 g, 78.38 mmol, 92.60% yield, 92% purity) as a yellow solid. LCMS (Method E): Rt=0.348 min, [M+H]⁺=341.1. ¹H NMR (400 MHz, DMSO-d₆) δ 8.61 (s, 1H), 8.12 (d, J=8.4 Hz, 1H), 7.98 (d, J=8.4 Hz, 1H), 7.30-7.23 (m, 1H), 7.09-7.02 (m, 2H), 6.90-6.83 (m, 1H), 5.47 (s, 2H), 4.16 (m, 2H), 2.79 (m, 2H), 2.34 (s, 6H).

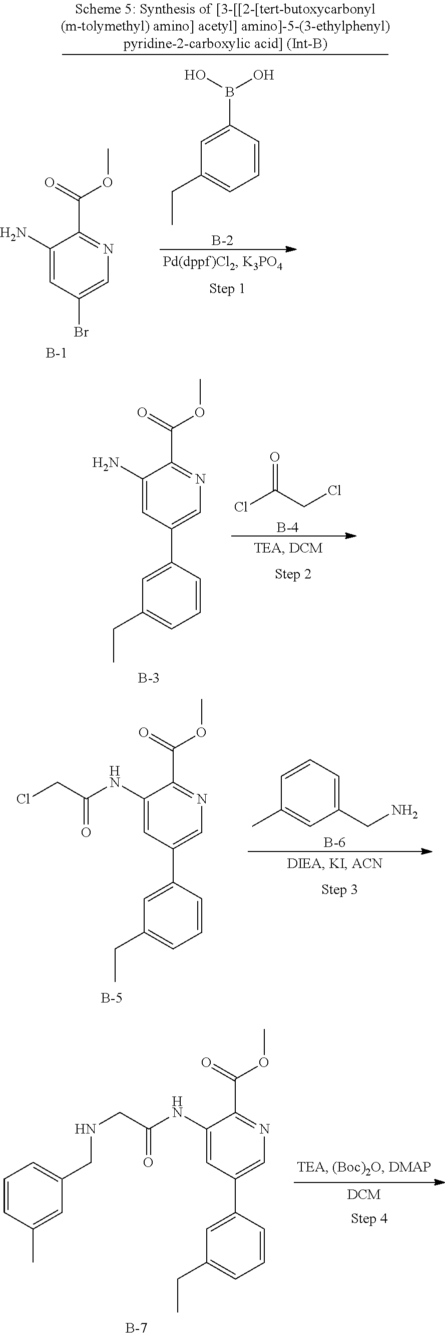

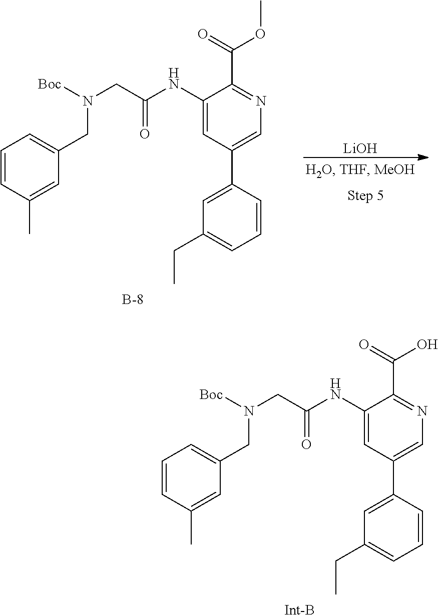

### Step 1: Synthesis of B-3

To a solution of B-1 (5 g, 21.64 mmol, 1 eq) and B-2 (3.89 g, 25.97 mmol, 1.2 eq) in dioxane (50 mL) and H₂O (10 mL) was added Pd(dppf)Cl₂ (1.41 g, 2.16 mmol, 0.1 eq) and K₃PO₄ (13.78 g, 64.92 mmol, 3 eq). The solution was degassed and purged with nitrogen 3 times. The mixture was stirred at 80° C. for 2 hr under nitrogen atmosphere. TLC (PE/EA=1:1. Rf=0.6) showed anew spot. The mixture was poured into water (60 mL) and extracted with EA (60 mL*3), the combined organic layers were dried over Na₂SO₄, filtered and concentrated under reduced pressure to give a residue. The residue was purified by column chromatography (SiO₂, petroleum ether/ethyl acetate=100/1 to 1/100) and concentrated under reduced pressure to give B-3 (4.3 g, 13.81 mmol, 63.84% yield, 82.34% purity) as a yellow solid. LCMS (Method D): Rt: 0.325 min, [M+H]⁺=257.0.

### Step 2: Synthesis of B-5

To a solution of B-3 (3.7 g, 14.44 mmol, 1 eq) in DCM (10 mL) was added TEA (5.84 g, 57.75 mmol, 8.04 mL, 4 eq) and B-4 (4.89 g, 43.31 mmol, 3.45 mL, 3 eq). The mixture was stirred at 0° C. for 1 hr. TLC (PE/EA=1:1. Rf=0.8) showed a new spot. The mixture was poured into water (18 mL) and extracted with DCM (18 mL*3). The combined organic layers were dried over Na₂SO₄, filtered and concentrated under reduced pressure to give a residue. The residue was purified by column chromatography (SiO₂, petroleum ether/ethyl acetate=100/1 to 1/100) and concentrated under reduced pressure to give B-5 (3.3 g, 9.60 mmol, 66.47% yield, 96.77% purity) as a white solid. LCMS (Method D): Rt: 0.429 min, [M+H]⁺=333.0.

### Step 3: Synthesis of B-7

To a solution of B-5 (3.8 g, 11.42 mmol, 1 eq) and B-6 (2.70 g, 17.13 mmol, 2.80 mL, 1.5 eq, HCl salt) in ACN (38 mL) was added DIEA (5.90 g, 45.68 mmol, 7.96 mL, 4 eq) and KI (7.58 g, 45.68 mmol, 4 eq). The mixture was stirred at 40° C. for 1 hr. The mixture was poured into water (40 mL), extracted with DCM (40 mL*3), and the combined organic layers were dried over Na₂SO₄, filtered and concentrated under reduced pressure to give B-7 (5 g, crude) as a yellow solid which was used for the next step without further purification. LCMS (Method D): Rt=0.341 min, [M+H]⁺=418.3.

### Step 4: Synthesis of B-8

To a solution of B-7 (5 g, 11.98 mmol, 1 eq) in DCM (50 mL) was added TEA (1.21 g, 11.98 mmol, 1.67 mL, 1 eq), DMAP (146.31 mg, 1.20 mmol, 0.1 eq) and Boc₂O (5.23 g, 23.95 mmol, 5.50 mL, 2 eq). The mixture was stirred at 25° C. for 1 hr. TLC (PE/EA=1:1, Rf=0.8) showed a new spot. The mixture was poured into water (50 mL), extracted with DCM (50 mL*3), and the combined organic layers were dried over Na₂SO₄, filtered, and concentrated under reduced pressure to give a residue. The residue was purified by column chromatography (SiO₂, petroleum ether/ethyl acetate=100/1 to 1/100) and concentrated under reduced pressure to give B-8 (4.4 g, 8.50 mmol, 70.98% yield, 99% purity) as a yellow solid. LCMS (Method D): Rt=0.570 min, [M+H]⁺=518.2.

### Step 5: Synthesis of Int-B

To a solution of B-8 (4.4 g, 8.50 mmol, 1 eq) in THF (15 mL), H₂O (15 mL) and MeOH (15 mL) was added LiOH·H₂O (1.07 g, 25.50 mmol, 3 eq) and the mixture was stirred at 25° C. for 0.5 hr. The mixture was poured into water (40 mL) and extracted with DCM (40 mL*2), then the pH of the aqueous phase was adjusted to 4-5 with 1 N HCl, extracted with DCM (20 mL*3), and then the combined organic layers were dried over Na₂SO₄, filtered and concentrated under reduced pressure to give Int-B (3.4 g, 6.75 mmol, 79.43% yield, 99% purity) as a yellow solid. LCMS (Method D): Rt=0.479 min, [M+H]⁺=504.2. ¹H NMR (400 MHz, DMSO-d₆) δ 14.31 (s, 1H), 9.40 (d, J=2.0 Hz, 1H), 8.62 (d, J=1.6 Hz, 1H), 7.68-7.59 (m, 3H), 7.48 (d, J=7.2 Hz, 1H), 7.43-7.37 (m, 1H), 7.34-7.23 (m, 3H), 4.73 (s, 2H), 4.14 (s, 2H), 3.30 (s, 2H), 2.93-2.86 (m, 2H), 2.48 (s, 3H), 1.58 (s, 9H), 1.43 (t, J=7.6 Hz, 3H).

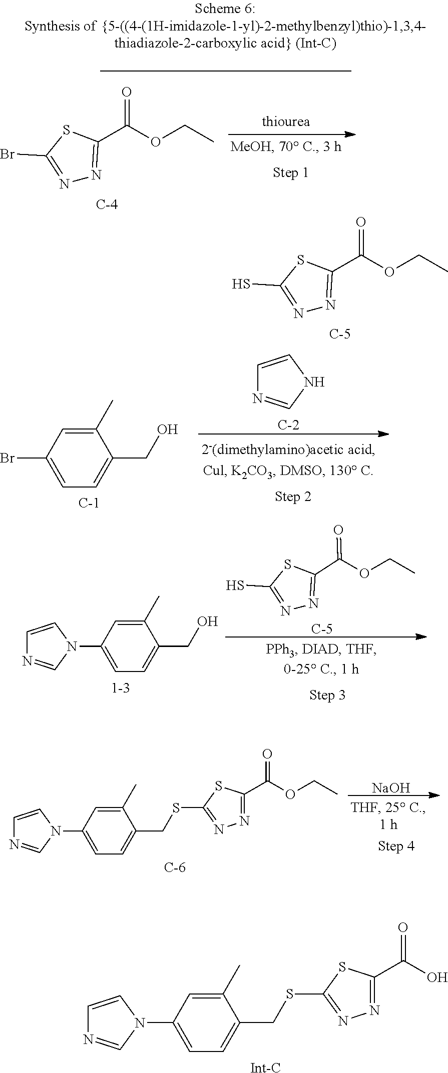

### Step 1: Synthesis of C-5

To a solution of C-4 (40 g, 168.72 mmol, 1 eq) in MeOH (160 mL) was added thiourea (19.27 g, 253.09 mmol, 1.5 eq) under N₂ and the reaction mixture was purged with N₂ three times then stirred at 70° C. for 6 hr. The reaction mixture was concentrated to remove MeOH, then acidified with 1 N aq. HCl (50 mL) at 0° C. and extracted with EA (50 mL*3). The combined organic phase was dried with anhydrous Na₂SO₄ and filtered. The filtrate was concentrated to give C-5 (32 g, 168.21 mmol, 99.69% yield) as a yellow solid which was used in the next step without any further purification. LCMS (Method E): Rt=0.445 min, [M+H]⁺+=191.1 ¹H NMR (400 MHz, DMSO-d₆) δ 4.34 (q, J=7.2 Hz, 2H), 1.29 (t, J=7.2 Hz, 3H).

### Step 2: Synthesis of C-3

To a solution of C-1 (50 g, 248.68 mmol, 1 eq) and C-2 (33.86 g, 497.36 mmol, 2 eq) in DMSO (500 mL) was added 2-(dimethylamino)acetic acid (2.56 g, 24.87 mmol, 0.1 eq), K₂CO₃ (34.37 g, 248.68 mmol, 1 eq), and CuI (9.47 g, 49.74 mmol, 0.2 eq) under N₂ and the reaction mixture was purged with N₂ three times and then stirred at 130° C. for 16 hr. The mixture was poured into saturated aq. NaCl (2000 mL) and then filtered and the filter cake was washed with EA (50 mL*3), and then the filtrate extracted with EA (500 mL*8). The combined organic layer was dried with anhydrous Na₂SO₄, filtered and evaporated to afford the crude product. The crude product was triturated with EA (30 mL), filtered and the filter cake was washed with EA (5 mL*3) to give C-3 (31 g, 164.70 mmol, 66.23% yield) as an off-white solid. LCMS (Method E): Rt=0.284 min, [M+H]⁺+=189.2. ¹H NMR (400 MHz, DMSO-d₆) δ 8.22 (br s, 1H), 7.71 (br s, 1H), 7.48-7.40 (m, 3H), 7.09 (br s, 1H), 5.21-5.13 (m, 1H), 4.52 (d, J=5.4 Hz, 2H), 2.30 (s, 3H).

### Step 3: Synthesis of C-6

To a solution of C-3 (21 g, 111.57 mmol, 1 eq) and C-5 (21.23 g, 111.57 mmol, 1 eq) in THF (200 mL) was added PPh₃ (35.12 g, 133.88 mmol, 1.2 eq) at 0° C. under N₂, and then DIAD (27.07 g, 133.88 mmol, 25.96 mL, 1.2 eq) in THF (60 mL) was added dropwise to the mixture at 0-10° C. and then stirred at 0-25° C. for 50 min under N₂. The reaction mixture was quenched with water (1000 mL) and extracted with EA (100 mL*3). The combined organic phase was dried with anhydrous Na₂SO₄, filtered and concentrated to give the crude product. The crude product was triturated with DCM (30 mL), filtered and the filter cake was washed with DCM (8 mL*3). The filtrate was purified by flash silica gel chromatography (330 g*2 SepaFlash silica flash column; eluent: 0-100% PE/EA) and the eluent was concentrated to give C-6 (36 g, 99.87 mmol, 89.52% yield) as a yellow solid. LCMS: Rt=0.561 min, [M+H]⁺+=360.9. ¹H NMR (400 MHz, DMSO-d₆) δ 8.25 (br s, 1H), 7.74 (s, 1H), 7.63-7.52 (m, 2H), 7.46-7.45 (m, 1H), 7.10 (br s, 1H), 4.72 (s, 2H), 4.40 (q, J=7.2 Hz, 2H), 2.46 (s, 3H), 1.33 (t, J=7.2 Hz, 3H).

### Step 4: Synthesis of Int-C

To a solution of C-6 (30 g, 83.23 mmol, 1 eq) in THF (300 mL) was added NaOH (1 M, 91.55 mL, 1.1 eq) slowly, the reaction mixture was stirred at 25° C. for 1 hr. White solid was precipitation. The reaction mixture was filtered and the filter cake was washed by THF (5 mL*3), the filter cake was dried under vacuum to give Int-C (23 g, 64.72 mmol, 77.76% yield, Na salt) as white solid. LCMS: Rt: 0.389 min, [M+H]⁺=332.9. ¹H NMR (400 MHz, DMSO-d₆) δ=8.23 (s, 1H), 7.72 (s, 1H), 7.58-7.49 (m, 2H), 7.43-7.40 (m, 1H), 7.08 (s, 1H), 4.58 (s, 2H), 2.44 (s, 3H).

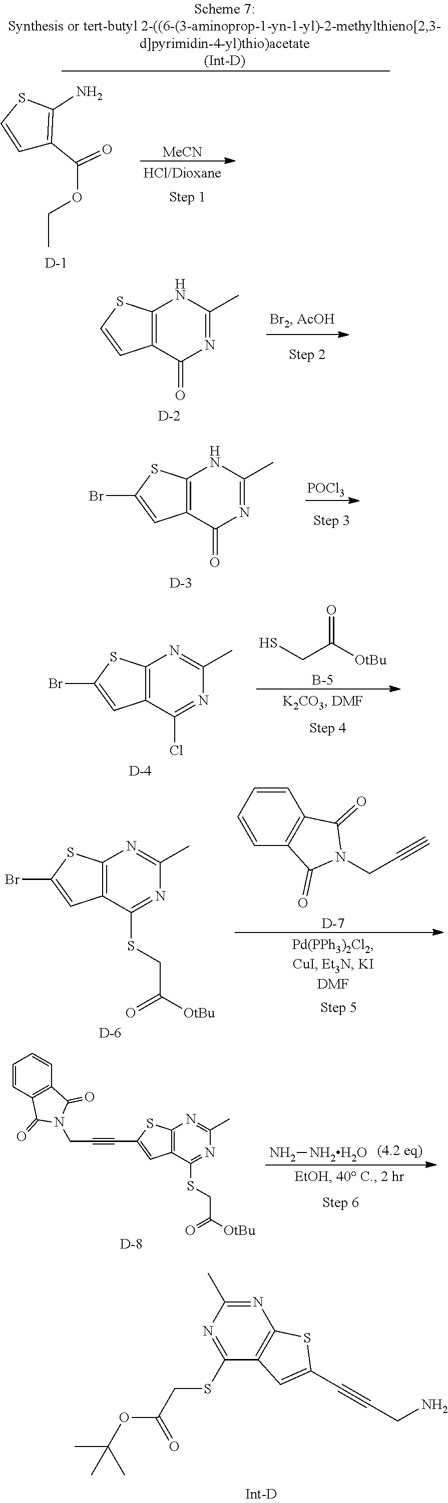

### Step 1: Synthesis of D-2

A mixture of D-1 (54 g, 315.39 mmol, 1 eq), MeCN (19.42 g, 473.09 mmol, 24.90 mL, 1.5 eq) and HCl/dioxane (4 M, 540.00 mL, 6.85 eq) was stirred at 20° C. for 30 mins. Then the mixture was stirred at 50° C. for 2 hrs and at 110° C. for 36 hrs. The reaction mixture was concentrated. The residue was triturated with MeCN (200 mL) at 20° C. 3 times to give D-2 (47 g, 282.79 mmol, 89.66% yield) as a yellow solid.

### Step 2: Synthesis of D-3

To a solution of D-2 (158 g, 950.66 mmol, 1 eq) in AcOH (1200 mL) was added Br₂ (182.31 g, 1.14 mol, 58.81 mL, 1.2 eq) dropwise at 20° C. The mixture was stirred at 20° C. for 16 hrs. The reaction mixture was concentrated to give a residue. The residue was diluted with H₂O (800 mL) and stirred for 30 mins. The mixture was filtered. The filter cake was washed with H₂O (800 mL×2) and dried under vacuum to give D-3 (77 g, crude) as a brown solid. LCMS (Method E): Rt=0.675 min, [M+H]⁺=244.9.

### Step 3: Synthesis of D-4

A suspension of D-3 (77 g, 314.16 mmol, 1 eq) in POCl₃ (495.00 g, 3.23 μmol, 300 mL, 10.28 eq) was stirred at 105° C. for 2 hrs. The reaction mixture was concentrated under vacuum to remove the POCl₃. The residue was poured into H₂O (300 mL) at 25° C. The mixture was neutralized with saturated aq. NaHCO₃ and extracted with EA (300 mL×3). The combined organic layer was washed with brine (300 mL), dried over Na₂SO₄, filtered and concentrated to give the crude product. The residue was purified by column chromatography (SiO₂, petroleum ether/ethyl acetate=20/1 to 10/1, PE/EA=5/1, R_{f}=0.7) and concentrated to give D-4 (35 g, 132.81 mmol, 42.27% yield, 100% purity) as a white solid. LCMS (Method E): Rt=0.887 min, [M+H]⁺=265.0. ¹H NMR (400 MHz, CDCl₃) δ 7.43 (s, 1H), 2.78 (s, 3H).

### Step 4: Synthesis of D-6

A mixture of D-4 (28 g, 106.24 mmol, 1 eq), D-5 (16.54 g, 111.56 mmol, 1.05 eq) and K₂CO₃ (44.05 g, 318.73 mmol, 3 eq) in DMF (300 mL) was stirred at 60° C. for 16 hrs. The reaction mixture was diluted with H₂O (200 mL) and extracted with EA (200 mL). The organic layer was washed with brine (200 mL×2), dried over Na₂SO₄, filtered and concentrated. The residue was purified by flash silica gel chromatography (30% EA in PE, PE/EA=5/1, R_{f}=0.4) to give D-6 (35 g, 93.26 mmol, 87.78% yield) as an off-white solid. LCMS (Method E): Rt=1.028 min, [M+H]⁺=375.1. ¹H NMR (400 MHz, CDCl₃) δ 7.32 (s, 1H), 4.01 (s, 2H), 2.69 (s, 3H), 1.48 (s, 9H).

### Step 5: Synthesis of D-8

To a solution of D-6 (1.0 g, 2.66 mmol, 1 eq), KI (1.33 g, 7.99 mmol, 3 eq) and TEA (1.35 g, 13.32 mmol, 1.85 mL, 5 eq) in DMF (10 mL) was added D-7 (740.11 mg, 4.00 mmol, 1.5 eq), Pd(PPh₃)₂Cl₂ (187.02 mg, 266.45 umol, 0.1 eq) and CuI (50.75 mg, 266.45 umol, 0.1 eq). After the addition, the reaction mixture was stirred at 80° C. for 2 hrs under N₂. The reaction mixture was cooled to 20° C., diluted with H₂O (50 mL) and extracted with EA (100 mL×2). The combined organic layer was washed with brine (100 mL), dried over Na₂SO₄, filtered and concentrated. The residue was purified by flash silica gel chromatography (40% EA in PE) to give D-8 (7 g, 14.60 mmol, 54.78% yield, 10 batches in parallel) as a yellow solid. LCMS (Method E): Rt=0.746 min, [M+H]⁺=480.1. ¹H NMR (400 MHz, CDCl₃) δ 7.92 (dd, J=3.2, 5.6 Hz, 2H), 7.76 (dd, J=3.2, 5.6 Hz, 2H), 7.38 (s, 1H), 4.73 (s, 2H), 3.99 (s, 2H), 2.70 (s, 3H), 1.47 (s, 9H).

### Step 6: Synthesis of Int-D

To a solution of D-8 (200 mg, 417.04 μmol, 1 eq) in EtOH (5 mL) was added hydrazine hydrate (87.68 mg, 1.75 mmol, 84.96 μL, 4.2 eq) at 25° C. The mixture was stirred at 40° C. for 2 hrs. The mixture was diluted with H₂O (5 mL) and extracted with EA (20 mL*3). The combined organic layers were washed with brine (15 mL*2), dried over anhydrous Na₂SO₄, filtered and concentrated under reduced pressure to give a residue. The residue was purified by flash silica gel chromatography (12 g SepaFlash silica flash column; eluent: 6% methanol/dichloromethane at 12 mL/min). Int-D (90 mg, 238.84 μmol, 57.27% yield, 92.740% purity) was obtained as a yellow oil. LCMS (Method G): Rt=0.668 min, [M+H]⁺=350.1. ¹H NMR (400 MHz, chloroform-d) δ 7.32 (s, 1H), 4.01 (s, 2H), 3.71 (m, 2H), 2.70 (s, 3H), 1.48 (s, 9H).

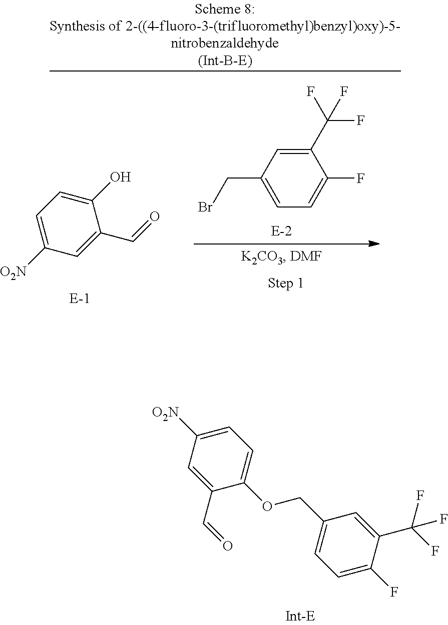 [SMILES: O=Cc1cc([N+](=O)[O-])ccc1O ; Fc1ccc(CBr)cc1C(F)(F)F ; O=Cc1cc([N+](=O)[O-])ccc1OCc1ccc(F)c(C(F)(F)F)c1]

### Step 1: Synthesis of Int-E

To a solution of E-1 (2 g, 11.97 mmol, 1 eq) and E-2 (3.38 g, 13.16 mmol, 1.1 eq) in DMF (20 mL) was added K₂CO₃ (4.96 g, 35.90 mmol, 3 eq). The mixture was stirred at 60° C. for 2 hrs. The residue was diluted with H₂O (20 mL) and extracted with ethyl acetate (25 mL*2). The combined organic phase was washed with brine (10 mL), dried with anhydrous Na₂SO₄, filtered and concentrated under vacuum. The crude residue was purified through flash silica gel chromatography (40 g SepaFlash silica flash column; eluent: 30-50% ethyl acetate/petroleum ether gradient) to give Int-E (2.5 g, 7.28 mmol, 60.86% yield) as a white solid, ¹H NMR (400 MHz, DMSO-d₆) δ=10.35 (s, 1H), 8.54 (dd, J=2.8, 9.2 Hz, 1H), 8.47 (d, J=3.2 Hz, 1H), 8.05-7.94 (m, 2H), 7.64-7.52 (m, 2H), 5.51 (s, 2H).

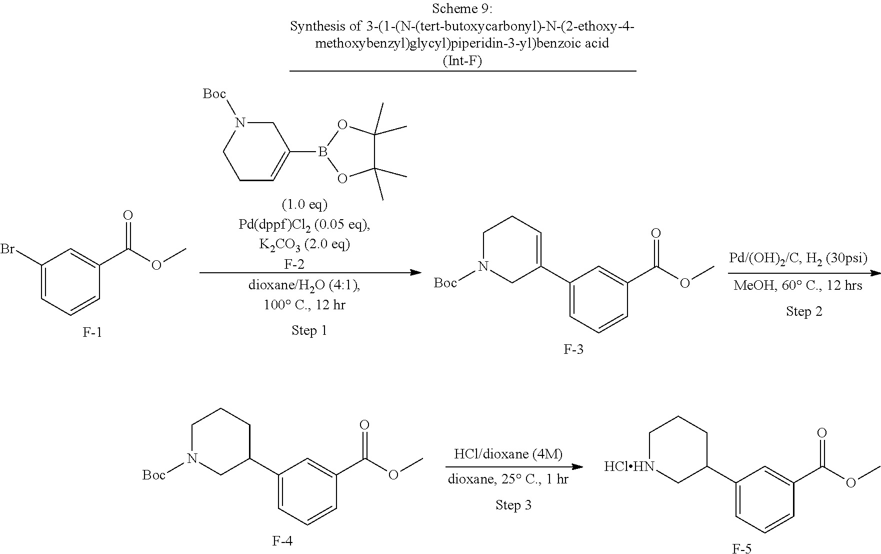

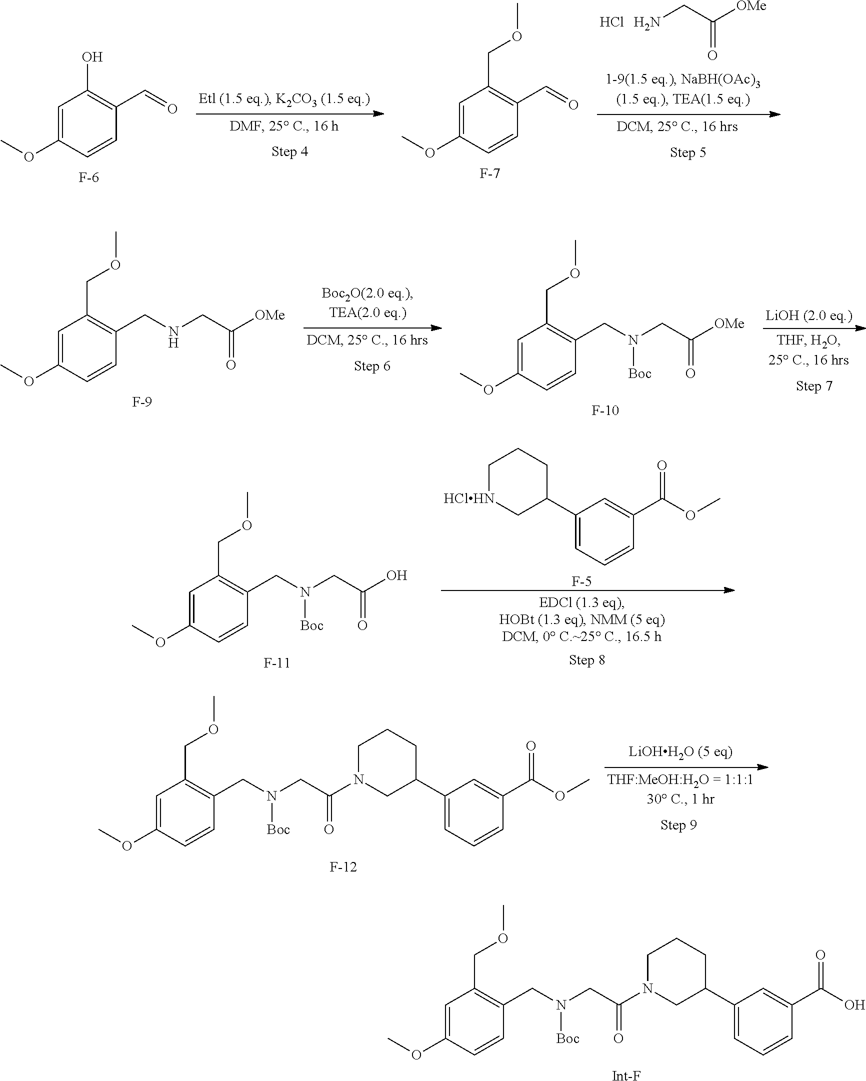

### Step 1: Synthesis of F-3

A mixture of F-1 (105 g, 488.27 mmol, 1 eq), F-2 (150.98 g, 488.27 mmol, 1 eq), Pd(dppf)Cl₂ (17.86 g, 24.41 mmol, 0.05 eq) and K₂CO₃ (134.96 g, 976.55 mmol, 2 eq) in dioxane (840 mL) and H₂O (210 mL) was degassed and purged with N₂ 3 times, and then the mixture was stirred at 100° C. for 12 hr under an N₂ atmosphere. The reaction was quenched by water (1 L), extracted with EA (1 L*3). The combined organic layers were concentrated to give a residue. The residue was divided into two parts and each part was purified by flash silica gel chromatography (:330 g SepaFlash silica flash column; eluent: 0-6% ethyl acetate/petroleum ether; gradient at 150 mL/min). Eluted fractions were concentrated to give F-3 (150 g, 463.17 mmol, 94.86% yield, 98% purity) as a colorless oil. LCMS (Method E): Rt=0.614 min, [M+Na]⁺=340.1. ¹H NMR (400 MHz, CDCl₃) δ 8.06 (s, 1H), 7.95 (d, J=7.6 Hz, 1H), 7.56 (br d, J=7.6 Hz, 1H), 7.46-7.34 (m, 1H), 6.28 (m, 1H), 4.30 (br s, 2H), 3.93 (s, 3H), 3.57 (m, 2H), 2.34 (m, 2H), 1.51 (s, 9H).

### Step 2: Synthesis of F-4

A mixture of F-3 (75 g, 236.31 mmol, 1 eq) and Pd(OH)₂/C (8.30 g, 11.82 mmol, 20% purity, 0.05 eq) in MeOH (1 L) was degassed and purged with H₂ 3 times, and then the mixture was stirred at 60° C. for 12 hr under H₂ (20 psi). The reaction mixture was filtered by diatomite (50 g), the filter cake washed with MeOH (100 mL*3) and the filtrate was concentrated to afford F-4 (75 g, 234.82 mmol, 99.37% yield, 99% purity) as a colorless oil. LCMS (Method E): Rt=0.612 min, [M+Na]⁺=342.1. ¹H NMR (400 MHz, CDCl₃) δ 8.02-7.85 (m, 2H), 7.52-7.34 (m, 2H), 4.26-4.12 (m, 2H), 3.92 (s, 3H), 2.84-2.67 (m, 3H), 2.02 (br s, 1H), 1.85-1.74 (m, 1H), 1.73-1.61 (m, 2H), 1.48 (s, 9H).

### Step 3: Synthesis of F-5

To a solution of F-4 (150 g, 469.64 mmol, 1 eq) in dioxane (750 mL) was added HCl/dioxane (4 M, 750 mL, 6.39 eq). The mixture was stirred at 25° C. for 1 hr. The reaction mixture was concentrated to give F-5 (108 g, 409.64 mmol, 87.22% yield, 97% purity, HCl salt) as a white solid. LCMS (Method E): Rt=0.371 min, [M+H]⁺=220.1. ¹H NMR (400 MHz, DMSO-d₆) δ 9.45-8.97 (m, 2H), 7.94-7.80 (m, 2H), 7.69-7.56 (m, 1H), 7.54-7.46 (m, 1H), 3.86 (s, 3H), 3.32-3.20 (m, 2H), 3.15-3.01 (m, 2H), 2.92 (br t, J=10.6 Hz, 1H), 1.94-1.65 (m, 4H).

### Step 4: Synthesis of F-7

To a solution of F-6 (180 g, 1.18 μmol, 1 eq) in DMF (900 mL) was added K₂CO₃ (245.26 g, 1.77 mol, 1.5 eq) and EtI (276.78 g, 1.77 μmol, 141.94 mL, 1.5 eq). The resulting mixture was allowed to reach 25° C. and stirred for 16 hr. The reaction mixture was quenched with water (4 L) and extracted by EA (1 L*3), and the combined organic layer were washed with brine (3 L*2) and concentrated under reduced pressure to afford the crude product. The crude product was diluted with EA (200 mL) and recrystallized to afford F-7 (210 g, 1.17 mol) as a white solid. LCMS (Method E): Rt: 0.500 min, [M+H]⁺=181.1. ¹H NMR (400 MHz, CDCl₃) δ 10.34 (s, 1H), 7.82 (d, J=8.4 Hz, 1H), 6.58-6.49 (m, 1H), 6.44 (d, J=2.0 Hz, 1H), 4.13 (q, J=6.8 Hz, 2H), 3.87 (s, 3H), 1.48 (t, J=6.8 Hz, 3H).

### Step 5: Synthesis of F-9

To a solution of F-7 (150 g, 832.41 mmol, 1 eq) in DCM (1500 mL) was added F-8 (156.77 g, 1.25 mol, 1.5 eq) and TEA (126.35 g, 1.25 mol, 173.79 mL, 1.5 eq). The resulting mixture was stirred for 0.5 hr at 25° C., then NaBH(OAc)₃ (264.63 g, 1.25 mol, 1.5 eq) was slowly added into the system under ice-bath. The whole system was then allowed to reach 25° C. and stirred for another 8 hr. The reaction mixture was quenched with water (3000 mL), the organic layer was separated and washed with brine (1000 mL). The combined organic phase was dried over anhydrous Na₂SO₄, and concentrated under reduced pressure to afford F-9 (290 g, crude) as a colorless oil.

### Step 6: Synthesis of F-10

To a solution of F-9 (290 g, crude, 1 eq) in DCM (2000 mL) was added TEA (231.71 g, 2.29 mol, 318.72 mL, 2.0 eq) and Boc₂O (499.75 g, 2.29 mol, 526.05 mL, 2.0 eq). The system was allowed to reach 25° C. and stirred for 16 hr. The reaction mixture was quenched with water (6000 mL) and extracted with EA (1500 mL*3). The combined organic layer was concentrated under reduced pressure to afford the crude product. The crude product was purified by silica gel chromatography (330 g SepaFlash silica flash column*4; eluent: 5-15% EA/PE) and the eluent was combined and concentrated to give F-10 (240 g, 679.10 mmol, 59.31% yield, 99% purity) as a colorless oil. LCMS (Method E): Rt: 0.623 min, LCMS: Rt: 0.598 min, [M+Na]⁺=376.1. ¹H NMR (400 MHz, CDCl₃) δ 7.25-7.09 (m, 1H), 6.51-6.33 (m, 2H), 4.52-4.35 (m, 2H), 4.04-3.86 (m, 4H), 3.79 (d, J=3.6 Hz, 3H), 3.70 (d, J=2.8 Hz, 3H), 1.46 (d, J=20.0 Hz, 9H), 1.39 (q, J=6.8 Hz, 3H).

### Step 7: Synthesis of F-11

To a solution of F-10 (120 g, 339.55 mmol, 1 eq) in THF (600 mL) was added LiOH·H₂O (14.25 g, 339.55 mmol, 1 eq) and H₂O (600 mL) in an ice-bath. The resulting mixture was then allowed to reach 25° C. and stirred for 16 hrs. The reaction mixture was concentrated under reduced pressure to remove THF (600 mL). The pH of the residue was adjusted to 6 by addition of aq. HCl (4.0 M). The resulting mixture was then extracted with EA (300 mL*3). The combined organic layer was then concentrated to afford the crude product. The crude product was recrystallized by EA (40 mL) to give F-11 (86 g, 253.40 mmol, 74.63% yield, 99% purity) as a white solid. LCMS (Method E): Rt=0.521 min, [M+Na]⁺=362.2. ¹H NMR (400 MHz, CDCl₃) δ 10.76-9.53 (m, 1H), 7.26-7.09 (m, 1H), 6.50-6.39 (m, 2H), 4.55-4.35 (m, 2H), 4.13-3.88 (m, 4H), 3.80 (s, 3H), 1.47 (br d, J=12.6 Hz, 9H), 1.43-1.37 (m, 3H).

### Step 8: Synthesis of F-12

To a solution of F-11 (113.86 g, 335.50 mmol, 1.1 eq) in DCM (800 mL) was added EDCI (76.01 g, 396.50 mmol, 1.3 eq), HOBt (53.58 g, 396.50 mmol, 1.3 eq) and NMM (154.25 g, 1.52 mol, 167.66 mL, 5 eq) at 0° C. The mixture was stirred at 0° C. for 0.5 hr. Then F-5 (78 g, 305.00 mmol, 1 eq, HCl salt) was added into the system. The reaction mixture was allowed to reach 25° C. and stirred for another 16 hrs. The reaction was quenched with water (300 mL) at 0° C. and extracted with DCM (100 mL*3). The combined organic layers were concentrated to afford a residue. The residue was divided into two parts and each part was purified by flash silica gel chromatography (: 330 g SepaFlash silica flash column; eluent of 0-32% ethyl acetate/petroleum ether; gradient at 150 mL/min). The eluent was combined and concentrated to give F-12 (149 g, 275.60 mmol, 90.36% yield, 99% purity) as a colorless oil. LCMS (Method E): Rt=0.622 min, [M+H]⁺=541.3. ¹H NMR (400 MHz, CDCl₃) δ 7.96-7.82 (m, 2H), 7.50-7.34 (m, 2H), 7.19-7.07 (m, 1H), 6.50-6.37 (m, 2H), 4.83-4.65 (m, 1H), 4.58-4.37 (m, 2H), 4.24-4.15 (m, 1H), 4.07-3.95 (m, 3H), 3.93 (s, 3H), 3.80 (br d, J=8.8 Hz, 4H), 3.10-2.93 (m, 1H), 2.86-2.68 (m, 1H), 2.67-2.52 (m, 1H), 2.09 (br s, 1H), 1.84 (br d, J=12.8 Hz, 1H), 1.79-1.67 (m, 1H), 1.63 (br d, J=5.2 Hz, 1H), 1.47 (br d, J=5.0 Hz, 9H), 1.43-1.34 (m, 3H).

### Step 9: Synthesis of Int-F

To a solution of F-12 (149 g, 275.60 mmol, 1 eq) in THF (500 mL) and MeOH (500 mL) was added a solution of LiOH·H₂O (57.83 g, 1.38 mol, 5 eq) in H₂O (500 mL). The mixture was stirred at 30° C. for 1 hr. The reaction mixture was concentrated under reduced pressure to remove the THF and MeOH at 30° C., and then cooled to 0° C. via ice-bath. After reaching 0° C., the pH was adjusted to 6 by addition of 1 N aq. HCl. The resulting mixture was extracted with EA (500 mL*3), and the combined organic layer was dried over anhydrous Na₂SO₄ and concentrated to afford Int-F (140 g, 263.19 mmol, 95.50% yield, 99% purity) as white solid. LCMS (Method E): Rt=0.566 min, [M+Na]⁺=549.3. ¹H NMR (400 MHz, CDCl₃) δ 13.18-12.63 (m, 1H), 7.94-7.74 (m, 2H), 7.65-7.35 (m, 2H), 7.06 (m, 1H), 6.62-6.37 (m, 2H), 4.62-4.16 (m, 3H), 4.07-3.91 (m, 4H), 3.72 (br d, J=9.2 Hz, 4H), 3.16-2.95 (m, 1H), 2.77-2.56 (m, 2H), 1.95-1.85 (m, 1H), 1.75 (br d, J=9.2 Hz, 2H), 1.54-1.24 (m, 13H).

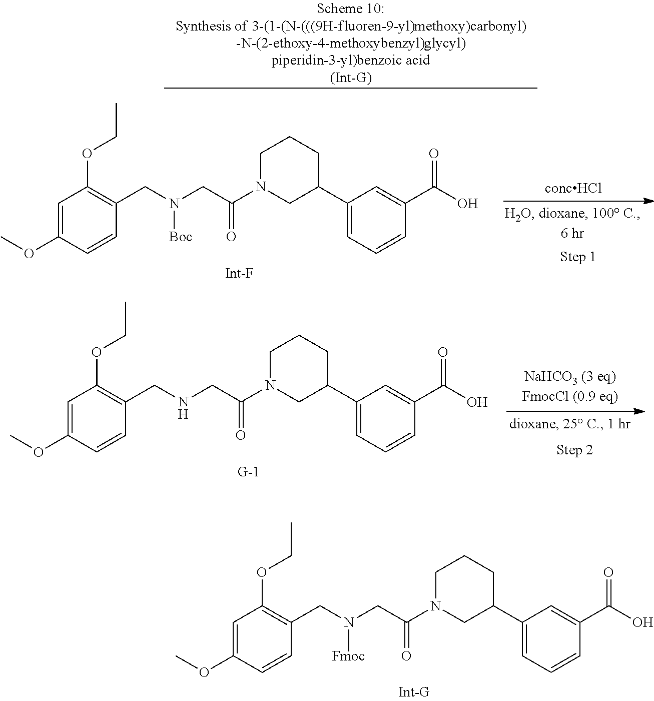

### Step 1: Synthesis of G-1

To a solution of conc. HCl (12 M, 7.99 mL, 1.01 eq) in H₂O (500 mL) and dioxane (50 mL) was added Int-F (50 g, 94.95 mmol, 1 eq). The mixture was stirred at 100° C. for 6 hr. The reaction mixture was cooled to 25° C. and used into next step directly. LCMS (Method E): Rt=0.444 min, [M+H]⁺=427.2.

### Step 2: Synthesis of Int-G

To the reaction mixture of G-1 was added dioxane (450 mL) and NaHCO₃ (23.93 g, 284.80 mmol, 11.08 mL, 3 eq), and then FmocCl (22.10 g, 85.44 mmol, 0.9 eq) was added. The mixture was stirred at 25° C. for 1 hr. The reaction mixture was adjusted to pH 6 with 1 N aq. HCl (70 mL). The resulting mixture was extracted with EA (500 mL*3), and the combined organic layer was dried over anhydrous Na₂SO₄ and concentrated to give Int-G (64 g, 86.81 mmol, two steps yield: 91.45%, 91.3% purity) as white solid. LCMS: Rt=0.607 min, [M+H]⁺=649.3. ¹H NMR (400 MHz, DMSO-d₆) δ 13.06-12.64 (m, 1H), 7.95-7.78 (m, 4H), 7.64-7.58 (m, 1H), 7.52 (br d, J=6.4 Hz, 2H), 7.48-7.35 (m, 3H), 7.29 (m, 2H), 7.02-6.68 (m, 1H), 6.58-6.49 (m, 1H), 6.49-6.30 (m, 1H), 4.50-4.15 (m, 6H), 4.12-3.88 (m, 4H), 3.73 (br d, J=8.0 Hz, 3H), 3.64-3.58 (m, 1H), 3.16-2.89 (m, 1H), 2.75-2.54 (m, 2H), 1.96-1.90 (m, 1H), 1.83-1.60 (m, 2H), 1.53-1.34 (m, 1H), 1.29 (m, 3H).

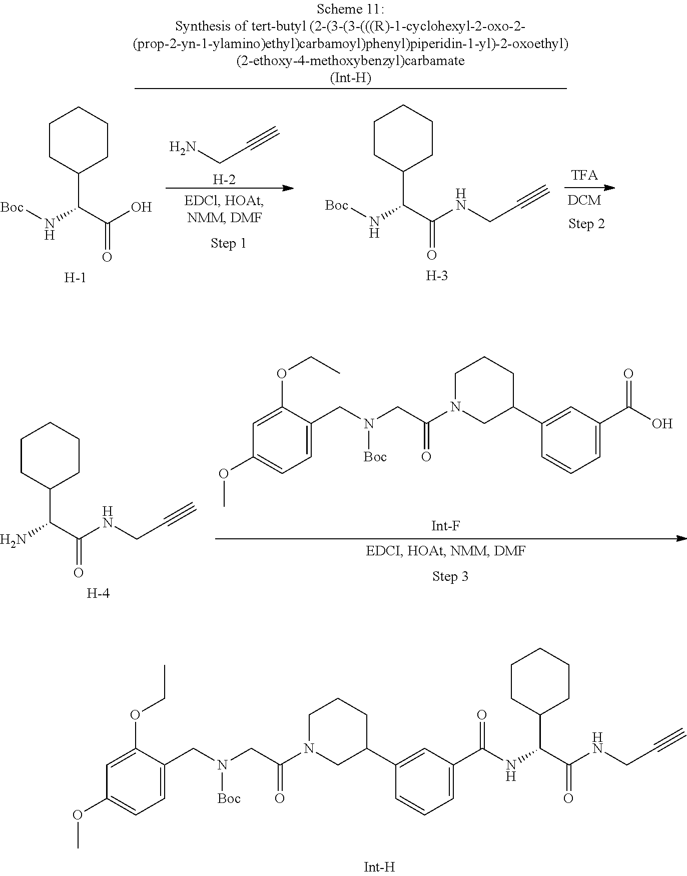

### Step 1: Synthesis of H-3

To a solution of H-1 (500 mg, 1.94 mmol, 1 eq) in DMF (5 mL) was added EDCI (1.12 g, 5.83 mmol, 3 eq), HOAt (264.47 mg, 1.94 mmol, 271.81 μL, 1 eq) and NMM (982.68 mg, 9.72 mmol, 1.07 mL, 5 eq) at 25° C. for 15 min. Then H-2 (139.13 mg, 2.53 mmol, 161.78 μL, 1.3 eq) was added at 25° C. The resulting mixture was stirred at 25° C. for 45 min. EA (4 mL) and water (4 mL) were added and the layers were separated. The aqueous phase was extracted with EA (4 mL×2), dried over Na₂SO₄, filtered, and concentrated under vacuum. The residue was purified by column chromatography (SiO₂, PE/EA=1/0 to 0/1, Rf=0.5 PE/EA=2:1) and the eluent was concentrated to give H-3 (400 mg, 1.36 mmol, 69.93% yield) as a white solid. ¹H NMR (400 MHz, CHLOROFORM-d) δ=6.19 (s, 1H), 5.08-4.94 (m, 1H), 4.09-3.99 (m, 2H), 3.93-3.85 (m, 1H), 2.26-2.21 (m, 1H), 1.77 (d, J=3.2 Hz, 5H), 1.45 (s, 9H), 1.26-0.92 (m, 6H).

### Step 2: Synthesis of H-4

To a solution of H-3 (150 mg, 509.53 μmol, 1 eq) in DCM (1 mL) was added TFA (460.50 mg, 4.04 mmol, 300.00 μL, 7.93 eq). The mixture was stirred at 25° C. for 0.5 hr. The mixture was concentrated under vacuum. The crude product was used in the next step without purification. H-4 (100 mg, 324.36 μmol, 63.66% yield, TFA) was obtained as a white solid. ¹H NMR (400 MHz, chloroform-d) δ 7.46-7.44 (m, 1H), 4.05-4.01 (m, 2H), 3.99 (d, J=3.2 Hz, 1H), 2.27-2.26 (m, 1H), 1.93-1.77 (m, 4H), 1.77-1.61 (m, 3H), 1.30-1.06 (m, 6H).

### Step 3: Synthesis of Int-H

To a solution of Int-F (153.73 mg, 291.93 μmol, 1 eq) in DMF (2 mL) was added EDCI (279.82 mg, 1.46 mmol, 5 eq), HOAt (79.47 mg, 583.85 μmol, 81.67 μL, 2 eq) and NMM (295.28 mg, 2.92 mmol, 320.95 μL, 10 eq) at 25° C., and the mixture was stirred at 25° C. for 0.5 hr. Then H-4 (90 mg, 291.93 mol, 1 eq, TFA) was added to the mixture at 25° C., and the reaction mixture was stirred at 25° C. for 1 hr. DCM (4 mL) and water (4 mL) were added and the layers were separated. The aqueous phase was extracted with DCM (5 mL×2), dried over Na₂SO₄, filtered, and concentrated to give a residue. The residue was purified by column chromatography (SiO₂, PE/EA=1/0 to 0/1) and the eluent was concentrated to give Int-H (175 mg, 241.70 μmol, 82.79% yield, 97.077% purity) as colorless oil. LCMS (Method D): Rt=0.546 min, [M+H]⁺=703.6. SFC (Method J): Rt=0.1.809 min, 2.024 min. ¹H NMR (400 MHz, chloroform-d) δ 7.64 (s, 2H), 7.37 (s, 1H), 7.19-7.08 (m, 1H), 7.01-6.93 (m, 1H), 6.42 (s, 2H), 4.69 (d, J=12.0 Hz, 1H), 4.56-4.38 (m, 3H), 4.19-4.06 (m, 2H), 4.01-3.97 (m, 4H), 3.78 (d, J=9.2 Hz, 4H), 3.13-2.98 (m, 2H), 2.64-2.52 (m, 1H), 2.21 (s, 1H), 2.03 (d, J=4.0 Hz, 3H), 1.89-1.73 (m, 6H), 1.58 (s, 2H), 1.46 (d, J=4.0 Hz, 9H), 1.42-1.33 (m, 3H), 1.33-1.02 (m, 6H).

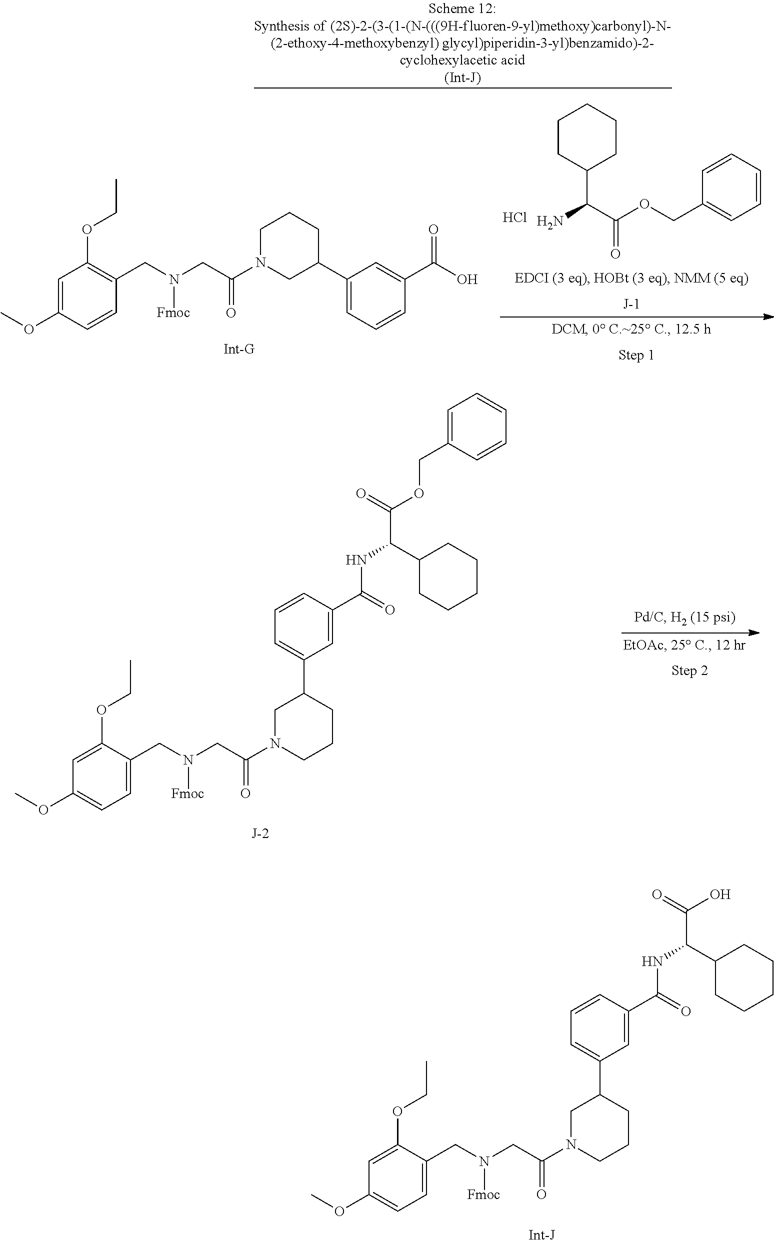

### Step 1: Synthesis of J-2

To a solution of Int-G (34 g, 46.12 mmol, 32.80 μL, 1 eq) in DCM (340 mL) was added EDCI (26.52 g, 138.36 mmol, 3 eq), HOBt (18.70 g, 138.36 mmol, 3 eq) and NMM (23.33 g, 230.60 mmol, 25.35 mL, 5 eq) at 0° C. The mixture was stirred at 0° C. for 0.5 hr. Then J-1 (17.02 g, 59.96 mmol, 1.3 eq, HCl salt) was added into the system. The reaction mixture was allowed to reach 25° C. and stirred for another 12 hrs. The reaction was quenched with water (300 mL). The resulting mixture was extracted by DCM (200 mL*3) and the combined organic layer was dried over anhydrous Na₂SO₄ and concentrated to give a residue. The residue was purified by flash silica gel chromatography (330 g SepaFlash silica flash column; eluent: 0-70% ethyl acetate/petroleum ether gradient at 120 mL/min). The eluent was concentrated under reduce pressure to give J-2 (36 g, 39.36 mmol, 85.34% yield, 96% purity) as white solid. LCMS (Method E): Rt=0.735 min, [M+H]⁺=878.7. SFC: Rt=2.459 min, 5.477 min. ¹H NMR (400 MHz, DMSO-d₆) δ 8.73-8.53 (m, 1H), 7.93-7.81 (m, 2H), 7.74 (br s, 2H), 7.65-7.49 (m, 2H), 7.48-7.21 (m, 11H), 6.99-6.67 (m, 1H), 6.57-6.28 (m, 2H), 5.23-5.05 (m, 2H), 4.49-4.29 (m, 4H), 4.29-4.16 (m, 3H), 4.13-3.95 (m, 4H), 3.73 (d, J=10.0 Hz, 3H), 3.67-3.53 (m, 1H), 3.15-2.85 (m, 1H), 2.73-2.55 (m, 2H), 1.96-1.83 (m, 2H), 1.81-1.53 (m, 7H), 1.50-1.34 (m, 1H), 1.33-1.24 (m, 3H), 1.16-0.96 (m, 4H).

### Step 2: Synthesis of Int-J

To a flask purged with nitrogen was added Pd/C (2.18 g, 10% purity) then EtOAc (500 mL) was added into the system under inert atmosphere. Then J-2 (36 g, 39.36 mmol, 1 eq) was added into the system. The system was degassed and purged with H₂, then the reaction mixture was stirred at 25° C. under H₂ atmosphere (15 psi) for 12 hr. The reaction mixture was filtered by celite (10 g), the filter cake was washed with EA (50 mL*3) and the filtrate was concentrated to give Int-J (30 g, 35.41 mmol, 89.96% yield, 93% purity) as a white solid. LCMS (Method E): Rt=0.649 min, [M+H]⁺=788.4. ¹H NMR (400 MHz, DMSO-d₆) δ12.82-12.43 (m, 1H), 8.54-8.33 (m, 1H), 7.94-7.81 (m, 2H), 7.76 (br s, 2H), 7.66-7.50 (m, 2H), 7.49-7.35 (m, 4H), 7.35-7.20 (m, 2H), 7.02-6.65 (m, 1H), 6.58-6.31 (m, 2H), 4.50-4.16 (m, 7H), 4.07-3.90 (m, 4H), 3.73 (d, J=8.6 Hz, 3H), 3.69-3.53 (m, 1H), 3.18-2.88 (m, 1H), 2.75-2.54 (m, 2H), 1.97-1.81 (m, 2H), 1.73 (br d, J=12.8 Hz, 6H), 1.64-1.54 (m, 1H), 1.52-1.34 (m, 1H), 1.33-1.25 (m, 3H), 1.17-1.02 (m, 4H).

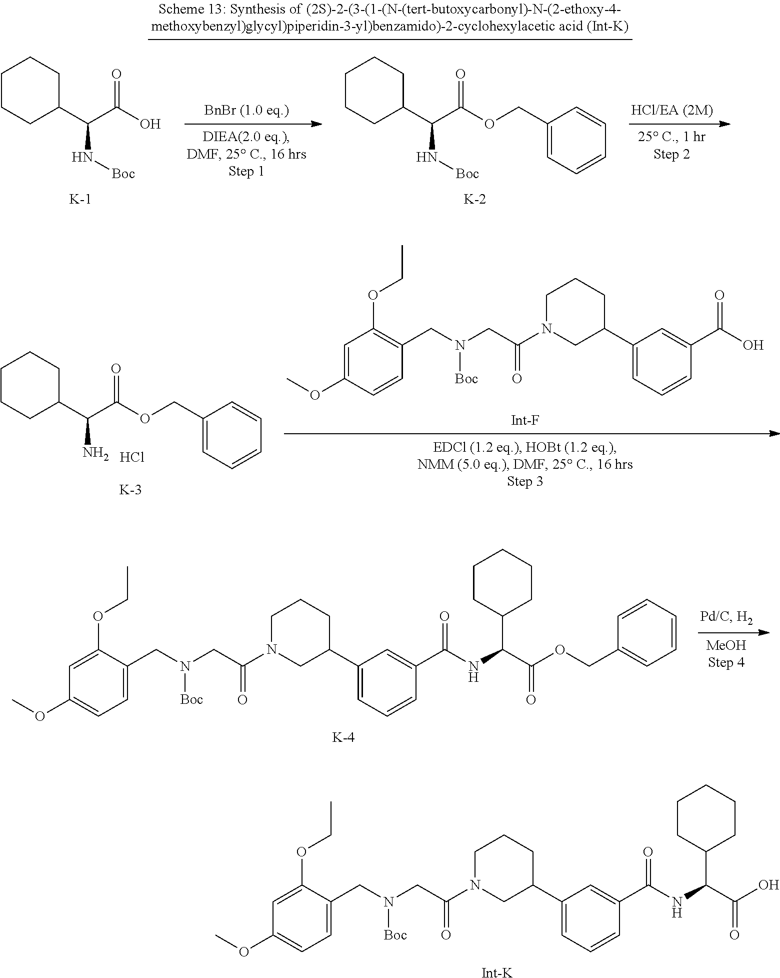

### Step 1: Synthesis of K-2

To a solution of K-1 (50 g, 194.31 mmol, 1 eq) in DMF (500 mL) was added DIEA (50.23 g, 388.61 mmol, 67.69 mL, 2 eq) and BnBr (33.23 g, 194.31 mmol, 23.08 mL, 1 eq). The reaction mixture was allowed to reach 25° C. and stirred for 16 hr. The reaction was quenched with water (2 L), then extracted with EA (500 mL*3). The combined organic layer was dried over anhydrous Na₂SO₄ and then concentrated under reduced pressure (via vacuum pump) to afford the crude product. The crude product was purified by flash silica gel chromatography (330 g SepaFlash silica flash column*2, eluent: 5-15% EA/PE) and the eluent was concentrated to give K-2 (50 g, 133.26 mmol, 68.58% yield, 92.60% purity) as a white solid. LCMS (Method E): Rt=0.658 min, [M+Na]⁺=370.3. ¹HNMR: (400 MHz, CDCl₃) δ 7.43-7.30 (m, 5H), 5.25-5.10 (m, 2H), 5.07-4.98 (m, 1H), 4.26 (br dd, J=4.9, 8.9 Hz, 1H), 1.85-1.51 (m, 7H), 1.44 (s, 9H), 1.30-1.10 (m, 4H).

### Step 2: Synthesis of K-3

K-2 (25 g, 71.95 mmol, 1 eq) was dissolved in EtOAc (125 mL) and added HCl/EtOAc (4N, 125 mL) was added drop wise. The resulting mixture was stirred at 25° C. for 1 hr. The reaction mixture was concentrated via vacuum pump to give K-3 (25 g, HCl salt form) as white solid. LCMS (Method E): Rt=0.468 min, [M+H]⁺=2482. ¹H NMR (400 MHz, CDCl₃) δ 8.926 (s, 3H), 7.46-7.30 (m, 5H), 5.34-5.16 (m, 2H), 2.14-2.01 (m, 2H), 1.89-1.70 (m, 3H), 1.69-1.56 (m, 2H), 1.51-1.02 (m, 7H).

### Step 3: Synthesis of K-4

To a solution Int-F (20 g, 37.98 mmol, 1 eq) in DMF (250 mL) was added EDCI (9.46 g, 49.37 mmol, 1.3 eq), HOBt (6.67 g, 49.37 mmol, 1.3 eq) and NMM (19.21 g, 189.89 mmol, 20.88 mL, 5 eq) at 0° C. and the resulting mixture was stirred for 0.5 hr before K-3 (10.78 g, 37.98 mmol, 1.0 eq, HCl) was introduced into the system. The reaction mixture was quenched with water (2 L) and extracted with EA (500 mL*3). The combined organic layer was washed with brine (800 mL*2) and dried over anhydrous Na₂SO₄ and then concentrated under reduced pressure (via vacuum pump) to afford the crude product. The crude was purified by flash silica-gel chromatography (330 g SepaFlash silica flash column*3, eluent: 55-70% EA/PE) and the eluent was concentrated to give K-4 (20 g, 24.34 mmol, 64.09% yield, 92% purity) as colorless oil: LCMS (Method E): Rt=0.734 min, [M+Na]⁺=778.5. ¹H NMR (400 MHz, CDCl₃) δ 7.75-7.57 (m, 2H), 7.38-7.31 (m, 7H), 7.24-7.05 (m, 1H), 6.79-6.58 (m, 1H), 6.49-6.36 (m, 2H), 5.25-5.13 (m, 2H), 4.85-4.77 (m, 1H), 4.76-4.64 (m, 1H), 4.62-4.33 (m, 2H), 4.04-3.90 (m, 3H), 3.83-3.68 (m, 4H), 3.08-2.94 (m, 1H), 2.80-2.47 (m, 2H), 1.97-1.87 (m, 1H), 1.84-1.52 (m, 10H), 1.51-1.32 (m, 12H), 1.20-0.98 (m, 5H).

### Step 4: Synthesis of Int-K

To a flask purged with nitrogen was added Pd/C (1.00 g, 939.67 μmol, 10% purity) and then EtOAc (100 mL) was added into the system. To the resulting mixture was then added K-4 (10 g, 13.23 mmol, 1 eq). The system was degassed and purged with H₂, and then the reaction mixture was stirred at 25° C. under H₂ atmosphere (15 psi) for 1 hr. The reaction mixture was filtered under inert atmosphere. The filter cake was washed with MeOH (50 mL*2). The filtrate was concentrated under reduced pressure to afford Int-K (15 g, 22.07 mmol, 83.41% yield, 97.95% purity) as white solid. LCMS (Method E): Rt=0.605 min, [M+H]⁺=666.5. SFC: Rt1=0.639, Rt2=1.292. ¹H NMR (400 MHz, CDCl₃) δ ¹H NMR (400 MHz, CDCl₃) δ 10.07 (br s, 1H), 7.73-7.66 (m, 1H), 7.65-7.42 (m, 1H), 7.40-7.03 (m, 4H), 6.49-6.33 (m, 2H), 4.79-4.63 (m, 2H), 4.60-4.06 (m, 4H), 4.04-3.88 (m, 3H), 3.65-3.80 (m, 3H), 3.04-2.97 (m, 1H), 2.80-2.48 (m, 2H), 2.03-1.91 (m, 2H), 1.87-1.58 (m, 8H), 1.52-1.33 (m, 12H), 1.24-1.01 (m, 5H).

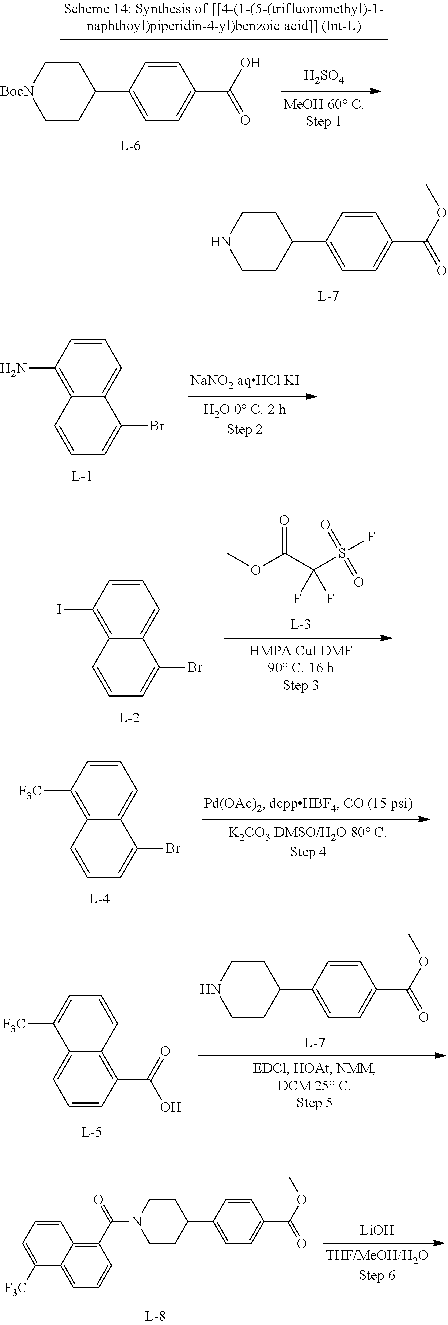

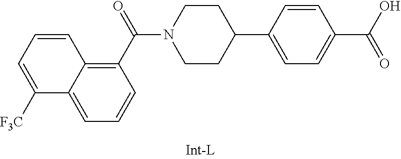 [SMILES: O=C(O)c1ccc(C2CCN(C(=O)c3cccc4c(C(F)(F)F)cccc34)CC2)cc1]

### Step 1: Synthesis of L-7

The mixture of L-6 (45 g, 147.36 mmol, 1 eq) in MeOH (500 mL) and conc. H₂SO₄ (25 mL) was stirred at 70° C. for 12 hr. The reaction was filtered and concentrated under reduced pressure to give L-7 (44 g, 134.60 mmol, 91.34% yield, 97.08% purity, H₂SO₄ salt) as a white solid which was used into the next step without further purification. LCMS (Method E): Rt=0.367 min, [M+H]⁺=220.2. ¹H NMR (400 MHz, DMSO-d₆) δ 8.65-8.19 (m, 2H), 7.93 (d, J=8.4 Hz, 2H), 7.39 (d, J=8.4 Hz, 2H), 3.84 (s, 3H), 3.43-3.38 (m, 1H), 3.34 (s, 1H), 3.09-2.89 (m, 3H), 2.02-1.90 (m, 2H), 1.88-1.73 (m, 2H).

### Step 2: Synthesis of L-2

To a solution of L-1 (85 g, 382.74 mmol, 1 eq) in conc. HCl (900 mL) was added a solution of NaNO₂ (52.81 g, 765.49 mmol, 2 eq) in H₂O (130 mL), the mixture was stirred at 0° C. for 0.5 h, and then a solution of KI (317.68 g, 1.91 μmol, 5 eq) in H₂O (250 mL) was added. The mixture was stirred at 0° C. for 0.5 h. The reaction was diluted with H₂O (3.0 L) and extracted with ethyl acetate (1 L*3). The combined organic phase was dried over Na₂SO₄, filtered and concentrated under reduced pressure to give a residue. The residue was purified by recrystallization from MeOH (500 mL) at 25° C. and filtered. The filter cake was dried under vacuum to give L-2 (90 g, 270.30 mmol, 70.62% yield) as a brown solid. ¹H NMR (400 MHz, DMSO-d₆) δ 8.23-8.21 (m, 1H), 8.17 (d, J=8.4 Hz, 1H), 8.06 (d, J=8.4 Hz, 1H), 7.96-7.94 (m, 1H), 7.56-7.52 (m, 1H), 7.42-7.38 (m, 1H).

### Step 3: Synthesis of L-4

A mixture of L-2 (80 g, 240.27 mmol, 1 eq), L-3 (230.79 g, 1.20 μmol, 152.84 mL, 5 eq), CuI (77.79 g, 408.45 mmol, 1.7 eq) and N-[bis(dimethylamino)phosphoryl]-N-methyl-methanamine (215.28 g, 1.20 mol, 210.23 mL, 5 eq) in DMF (800 mL) was degassed and purged with N₂ 3 times, and then the mixture was stirred at 90° C. for 16 hr under an N₂ atmosphere. The reaction was cooled to room temperature, then diluted with H₂O (2 L), extracted with ethyl acetate (1 L*3), and the combined organic phase was washed with saturated aqueous NaCl (2 L), dried over Na₂SO₄, filtered and concentrated under reduced pressure to give a residue. The residue was purified by flash silica gel chromatography (660 g SepaFlash silica flash column; eluent: 0-5% ethyl acetate/petroleum ether gradient at 200 mL/min). The eluent was concentrated to afford L-4 (100 g, crude) as a yellow solid. ¹H NMR (400 MHz, CDCl₃) δ 8.51 (d, J=8.4 Hz, 1H), 8.26-8.13 (m, 1H), 7.95-7.90 (m, 2H), 7.65-7.58 (m, 1H), 7.48-7.44 (m, 1H).

### Step 4: Synthesis of L-5

To a solution of 1-4 (50 g, 181.78 mmol, 1 eq) in DMSO (500 mL) and H₂O (50 mL) was added Pd(OAc)₂ (4.08 g, 18.18 mmol, 0.1 eq), 1,3-bis(dicyclohexylphosphino)propane bis(tetrafluoroborate) (22.26 g, 36.36 mmol, 0.2 eq) and K₂CO₃ (50.25 g, 363.55 mmol, 2 eq) under N₂. The suspension was degassed under vacuum and purged with CO three times. The mixture was stirred under CO (20 psi) at 80° C. for 16 hours. The reaction was diluted with H₂O (1 L) and then extracted with ethyl acetate (500 mL*2) to remove impurities. The aqueous phase was adjusted to pH=4 with saturated aqueous citric acid and extracted with EA (1 L*3). The combined organic phase was dried over Na₂SO₄, filtered and concentrated under reduced pressure to give a residue. The residue was purified by flash silica gel chromatography (660 g SepaFlash silica flash column; eluent: 0-60% ethyl acetate/petroleum ether gradient at 200 mL/min). The eluent was concentrated to afford L-5 (30 g, 124.91 mmol, 34.36% yield) as a yellow solid. LCMS: Rt=0.206 min, (M−H)=239.1. ¹H NMR (400 MHz, DMSO-d₆) δ 13.77-13.13 (m, 1H), 9.18 (d, J=8.8 Hz, 1H), 8.35 (d, J=8.8 Hz, 1H), 8.30 (d, J=7.2 Hz, 1H), 8.13 (d, J=7.2 Hz, 1H), 7.92-7.79 (m, 2H).

### Step 5: Synthesis of L-8

To a solution of L-5 (30 g, 124.91 mmol, 1 eq) and L-7 (39.64 g, 124.91 mmol, 1.0 eq, H₂SO₄ salt) in DCM (300 mL) was added EDCI (71.84 g, 374.72 mmol, 3 eq), NMM (63.17 g, 624.54 mmol, 68.66 mL, 5 eq) and HOAt (25.50 g, 187.36 mmol, 26.21 mL, 1.5 eq). The mixture was stirred at 25° C. for 1 hr. The reaction was diluted with H₂O (500 mL) and extracted with ethyl acetate (500 mL*3) and the combined organic phase was washed with saturated aqueous NaCl (500 mL), dried over Na₂SO₄, filtered and concentrated under reduced pressure to give a residue. The residue was purified by flash silica gel chromatography (660 g SepaFlash silica flash column; eluent: 0-100% ethyl acetate/petroleum ether gradient at 200 mL/min). The eluent was concentrated to afford L-8 (30 g, 61.16 mmol, 48.97% yield, 90% purity) as a yellow solid. LCMS (Method E): Rt=0.629 min, [M+H]⁺=442.2. ¹H NMR (400 MHz, CDCl₃) δ 8.25 (d, J=8.4 Hz, 1H), 8.21-8.03 (m, 1H), 8.01-7.98 (m, 2H), 7.96-7.88 (m, 1H), 7.73-7.45 (m, 3H), 7.34-7.23 (m, 2H), 5.17-5.10 (m, 1H), 3.92 (d, J=2.4 Hz, 3H), 3.63-3.41 (m, 1H), 3.22-3.06 (m, 1H), 3.05-2.94 (m, 1H), 2.90-2.83 (m, 1H), 2.18-2.04 (m, 1H), 1.97-1.81 (m, 1H), 1.80-1.68 (m, 1H), 1.63-1.39 (m, 1H).

### Step 6: Synthesis of Int-L

To a solution of L-8 (28 g, 63.43 mmol, 1 eq) in MeOH (200 mL), THF (100 mL) and H₂O (100 mL) was added LiOH·H₂O (7.99 g, 190.29 mmol, 3 eq). The mixture was stirred at 25° C. for 1 hr. The reaction mixture was concentrated under reduced pressure to give a residue. The residue was diluted with H₂O (200 mL) and extracted with ethyl acetate (100 mL*2) to remove impurities. The aqueous phase was adjusted to pH 4 with saturated aqueous citric acid and extracted with EA (200 mL*3). Then the combined organic phase was dried over Na₂SO₄, filtered and concentrated under reduced pressure to give Int-L (26 g, 60.26 mmol, 95.0% yield, 99.05% purity) as a white solid which was used in the next step without purification. LCMS (Method E): Rt=0.553 min, [M+H]⁺=428.2. ¹H NMR (400 MHz, DMSO-d₆) δ 13.45-12.05 (m, 1H), 8.28-8.02 (m, 3H), 7.89-7.86 (m, 2H), 7.84-7.55 (m, 3H), 7.43-7.39 (m, 2H), 4.88-4.81 (m, 1H), 3.37-3.24 (m, 1H), 3.23-3.06 (m, 1H), 3.05-2.80 (m, 2H), 2.03-1.91 (m, 1H), 1.90-1.70 (m, 1H), 1.67-1.14 (m, 2H).

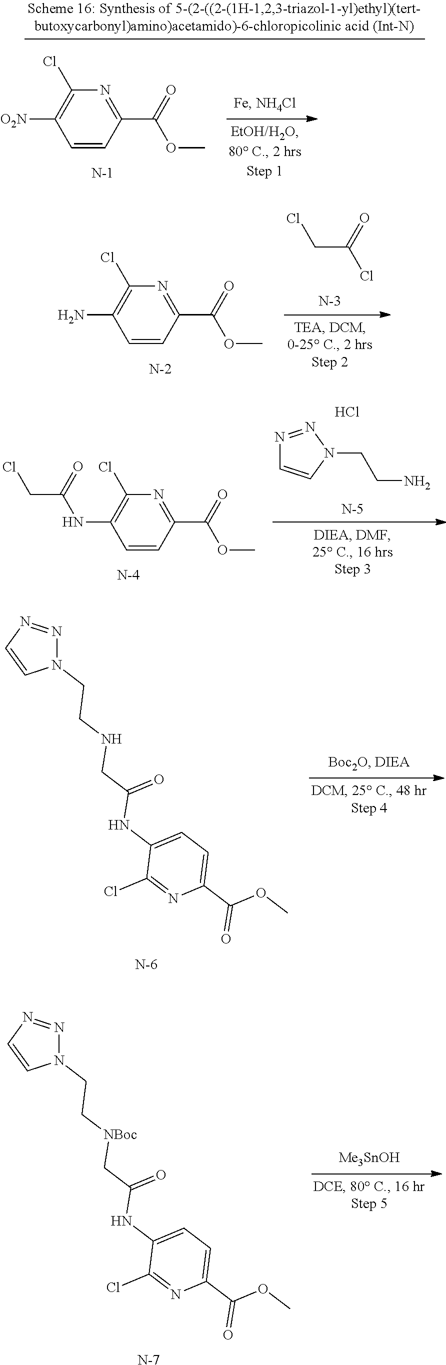

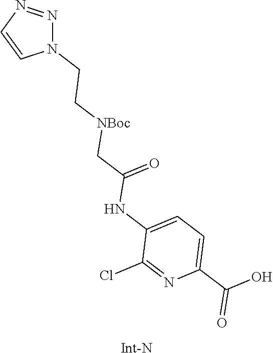 [SMILES: CC(C)(C)OC(=O)N(CCn1ccnn1)CC(=O)Nc1ccc(C(=O)O)nc1Cl ; "Int-N"]

### Step 1: Synthesis of N-2

To a solution of N-1 (60 g, 277.04 mmol, 1 eq), NH₄Cl (37.05 g, 692.59 mmol, 2.5 eq) in EtOH (400 mL) and H₂O (400 mL) was added Fe (77.36 g, 1.39 mol, 5 eq). The mixture was stirred at 80° C. for 1 hr. The reaction mixture was filtered and the filtrate was poured into water (200 mL) and extracted with EA (200 mL*2). The organic layer was washed with brine (20 mL), dried over Na₂SO₄, and concentrated to afford the crude product N-2 (52 g, 268.65 mmol, 96.97% yield, 96.4% purity) as a yellow solid. LCMS (Method E): Rt=0.360 min, [M+Na]⁺=208.9.

### Step 2: Synthesis of N-4

To a solution of N-2 (48 g, 257.24 mmol, 1 eq) and TEA (260.30 g, 2.57 mol, 358.05 mL, 10 eq) in DCM (500 mL) was added N-3 (174.32 g, 1.54 mol, 122.76 mL, 6 eq) at 0° C. Then the mixture was stirred at 25° C. for 16 hr. The reaction mixture was poured into water (100 mL) and extracted with DCM (100 mL*2). The organic layer was washed with brine (10 mL), dried over Na₂SO₄ and concentrated to afford the crude product. The crude product was purified by flash silica gel chromatography (SiO₂, PE:EA=10:1 to 3:1) to give N-4 (36 g, 109.88 mmol, 42.72% yield, 80.3% purity) as a yellow solid. LCMS (Method E): Rt=0.434 min, [M+H]⁺=262.9.

### Step 3: Synthesis of N-6

To a solution of N-4 (35 g, 133.04 mmol, 1 eq), N-5 (39.54 g, 266.08 mmol, 2 eq, HCl) in DMF (400 mL) was added DIEA (85.97 g, 665.20 mmol, 115.87 mL, 5 eq) and was allowed to stir at 25° C. for 16 hr. The reaction mixture was poured into water (600 mL) and extracted with EA (300 mL*4). The combined organic layers were washed with brine (300 mL), dried over Na₂SO₄ and concentrated to give the crude product. The crude product was purified by flash silica gel chromatography (SiO₂, PE:EA=5:1 to 1:1) to give N-6 (24 g, 70.07 mmol, 52.67% yield, 98.9% purity) as a yellow solid. LCMS (Method E): Rt=0.306 min, [M+H]⁺=339.0. ¹H NMR (400 MHz, DMSO-d₆) δ 8.80 (d, J=8.4 Hz, 1H), 8.17 (d, J=0.8 Hz, 1H), 8.11 (d, J=8.4 Hz, 1H), 7.73 (d, J=0.8 Hz, 1H), 4.54-4.52 (m, 2H), 3.88 (s, 3H), 3.42 (s, 2H), 3.13-3.11 (m, 2H).

### Step 4: Synthesis of N-7

To solution of N-6 (13 g, 38.38 mmol, 1 eq) and DIEA (14.88 g, 115.13 mmol, 20.05 mL, 3 eq) in DCM (130 mL) was added Boc₂O (16.75 g, 76.75 mmol, 17.63 mL, 2 eq). The mixture was stirred at 25° C. for 48 hr. The reaction mixture was concentrated to give the crude product, which was purified by flash silica gel chromatography (SiO₂, PE:EA=10:1 to 1:1) to give N-7 (13.5 g, 26.82 mmol, 69.90% yield, 87.2% purity) as white solid. LCMS (Method E): Rt=0.457 min, [M+H]⁺=439.1.

### Step 5: Synthesis of Int-N

To a solution of N-7 (16 g, 36.46 mmol, 1 eq) in DCE (160 mL) was added hydroxy(trimethyl)stannane (32.96 g, 182.29 mmol, 5 eq) and was allowed to stir at 80° C. for 16 hr. The reaction mixture was filtered and concentrated to give the crude product. The crude product was purified by flash silica gel chromatography (SiO₂, PE:EA=3:1 to 1:1) to give Int-N (15.5 g, 25.72 mmol, 70.55% yield, 70.5% purity) as white solid. LCMS (Method E): Rt=0.421 min, [M+H]⁺=425.2. ¹H NMR (400 MHz, DMSO-d₆) δ 9.79 (br d, J=8.8 Hz, 1H), 8.51-8.27 (m, 1H), 8.16 (s, 1H), 7.95 (br d, J=5.6 Hz, 1H), 7.73 (d, J=9.6 Hz, 1H), 4.56-4.54 (m, 2H), 4.12-4.00 (m, 2H), 3.72-3.70 (m, 2H), 1.39-1.18 (m, 9H).

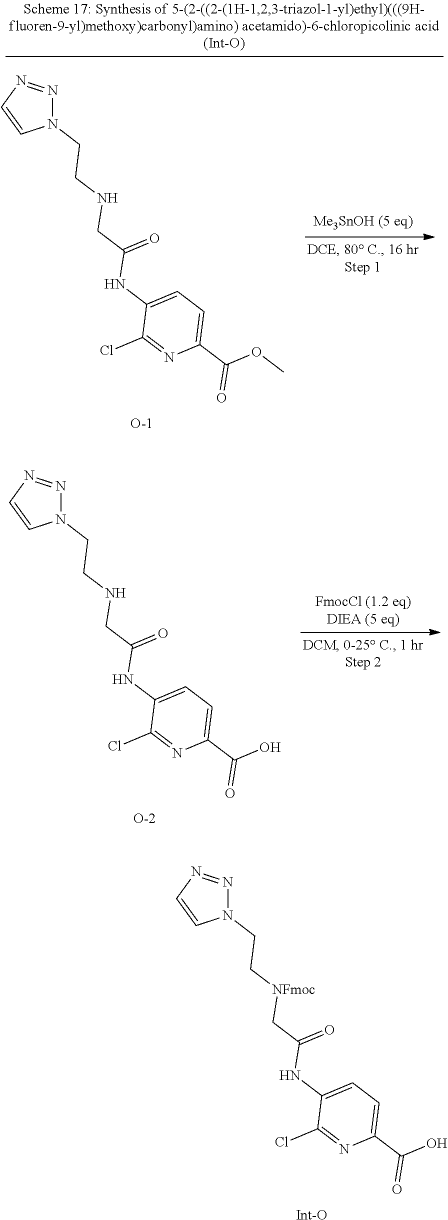

### Step 1: Synthesis of 0-2

To a solution of 0-1 (21 g, 61.99 mmol, 1 eq) in DCE (200 mL) was added hydroxy(trimethyl)stannane (56.05 g, 309.96 mmol, 5 eq). Then the mixture was stirred at 80° C. for 16 hr. The reaction mixture was filtered and the filtrate was concentrated to give the crude product 0-2 (20 g, 46.87 mmol, 75.61% yield, 76.1% purity) as yellow oil. LCMS (Method E): Rt=0.329 min, [M+H]⁺=325.

### Step 2: Synthesis of Int-O

To solution of 0-2 (20 g, 61.59 mmol, 1 eq), DIEA (39.80 g, 307.96 mmol, 53.64 mL, 5 eq) in DCE (200 mL) was added Fmoc-Cl (39.83 g, 153.98 mmol, 2.5 eq) and the mixture was allowed to stir at 0° C. for 1 hr. The reaction mixture was filtered and concentrated to give the crude product. The crude product was purified by flash silica gel chromatography (SiO₂, PE:EA=10:1 to 1:1) to give Int-0 (14.8 g, 25.90 mmol, 42.04% yield, 95.7% purity) as light yellow solid. LCMS: Rt=0.493 min, [M+H]⁺=547.2. ¹H NMR (DMSO-d₆, 400 MHz) δ 9.92 (br d, J=15.6 Hz, 1H), 8.50-8.48 (m, 1H), 8.25-7.98 (m, 2H), 7.94-7.71 (m, 4H), 7.68-7.54 (m, 2H), 7.45-7.31 (m, 3H), 7.17-7.15 (m, 1H), 4.60-4.58 (m, 1H), 4.38-4.18 (m, 4H), 4.16-4.01 (m, 2H), 3.79-3.77 (m, 1H), 3.65-3.63 (m, 1H).

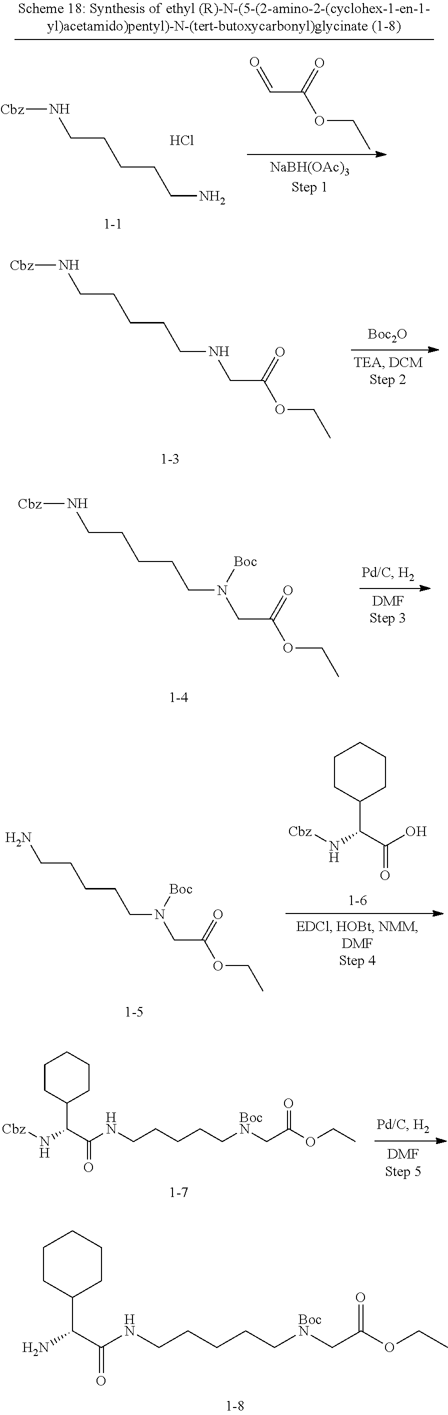

### Step 1: Synthesis of 1-3

1-1 (500 mg, 2.12 mmol, 1 eq) was added into 10% aq. K₂CO₃ (10 mL) and extracted with DCM (5 mL*3). The combined organic layers were dried over anhydrous sodium sulfate, filtered, and concentrated to give a residue. The residue was dissolved in TFE (5 mL), followed by addition of ethyl 2-oxoacetate (432.01 mg, 2.12 mmol, 1 eq) and 4 Å MS (100 mg). The reaction mixture was stirred at 25° C. for 1 h. NaBH(OAc)₃ (1.12 g, 5.29 mmol, 2.5 eq) was then added at 0° C. The reaction mixture was stirred at 25° C. for 1 h. The reaction mixture was filtered to remove solids. The filtrate was poured into 10% aq. K₂CO₃ (10 mL) and extracted with DCM (10 mL*3). The combined organic layers were washed with brine (10 mL), dried over anhydrous sodium sulfate, filtered, and concentrated to give a residue. The residue purified by flash silica gel chromatography (12 g SepaFlash silica flash column; eluent: 0-40% MeOH/ethyl acetate; gradient at 20 mL/min) and the eluent was concentrated to give 1-3 (200 mg, 620.35 μmol, 29.32% yield, 99% purity) as a yellow solid. LCMS (Method E): Rt=0.510 min, [M+H]⁺=323.0.

### Step 2: Synthesis of 1-4

To a solution of 1-3 (200 mg, 620.35 μmol, 1 eq) in DCM (2 mL) was added TEA (125.55 mg, 1.24 mmol, 172.69 μL, 2 eq) and Boc₂O (176.01 mg, 806.46 μmol, 185.27 μL, 1.3 eq). The reaction mixture was stirred at 25° C. for 2 h. The reaction mixture was diluted with DCM (1 mL) and purified by flash silica gel chromatography (20 g silica flash column; eluent of 0-40% ethyl acetate/petroleum ether, gradient at 30 mL/min) to afford 1-4 (190 mg, 427.20 μmol, 68.87% yield, 95% purity) as a colorless oil. LCMS (Method E): Rt=0.590 min, [M+Na]⁺=445.4. ¹H NMR (400 MHz, CDCl₃) δ 7.41-7.29 (m, 5H), 5.21-5.03 (m, 2H), 4.94-4.63 (m, 1H), 4.21-4.16 (m, 2H), 3.99-3.75 (m, 2H), 3.37-3.10 (m, 4H), 1.56-1.50 (m, 4H), 1.50-1.39 (m, 9H), 1.39-1.23 (m, 5H).

### Step 3: Synthesis of 1-5

To a solution of 1-4 (190 mg, 449.69 μmol, 1 eq) in DMF (2 mL) was added Pd/C (100 mg, 93.97 mol, 10% purity) and the suspension was degassed under vacuum and purged with H₂ (15 psi) three times. The mixture was stirred at 25° C. for 1 hr. The reaction mixture was concentrated under reduced pressure to give 1-5 (120 mg, crude), which was used into the next step without further purification. LCMS (Method E): Rt=0.398 min, [M+H]⁺=289.2.

### Step 4: Synthesis of 1-7

To a solution of 1-5 (700 mg, 2.43 mmol, 1.1 eq) and 1-6 (642.89 mg, 2.21 mmol, 1 eq) in DMF (7 mL) was added EDCI (1.27 g, 6.62 mmol, 3 eq), HOBt (298.17 mg, 2.21 mmol, 1 eq) and NMM (2.23 g, 22.07 mmol, 2.43 mL, 10 eq). The mixture was stirred at 25° C. for 2 hr. The reaction mixture was concentrated under reduced pressure to give the crude product. The product was purified by prep-HPLC (column: Phenomenex luna C18 150 mm*25 mm*10 um; mobile phase: [water(FA):ACN]; gradient: 57%-87% over 10 min) and the eluent was concentrated and then lyophilized to give 1-7 (600 mg, 1.04 mmol, 46.95% yield, 97% purity) as a colorless oil. LCMS (Method E): Rt=0.622 min, [M+H]⁺=562.4. SFC: Rt=1.332 min, ee=97.57%.

### Step 5: Synthesis of 1-8

To a solution of 1-7 (600 mg, 1.07 mmol, 1 eq) in DMF (6 mL) was added Pd/C (300 mg, 281.90 μmol, 10% purity). The suspension was degassed under vacuum and purged with H₂ (15 psi) three times. The mixture was stirred at 25° C. for 1 hr. The reaction mixture was concentrated under reduced pressure to give 1-8 (420 mg, 982.28 μmol, 91.96% yield, 99% purity) as a white solid which was used into the next step without further purification. LCMS (Method E): Rt=0.463 min, [M+H]⁺=428.2.

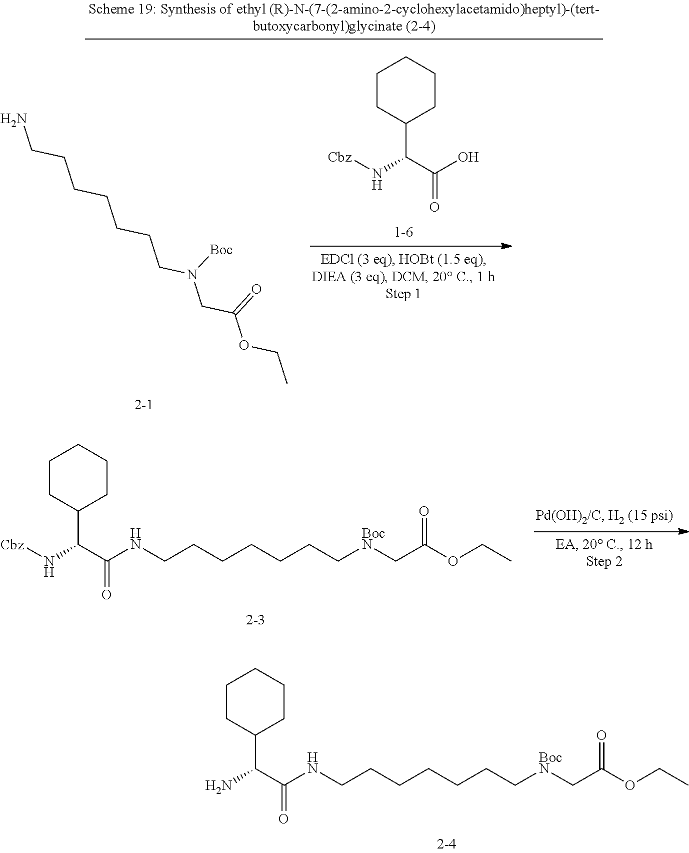 [SMILES: CCOC(=O)CN(CCCCCCCN)C(=O)OC(C)(C)C ; O=C(N[C@@H](C(=O)O)C1CCCCC1)OCc1ccccc1 ; CCOC(=O)CN(CCCCCCCNC(=O)[C@H](NC(=O)OCc1ccccc1)C1CCCCC1)C(=O)OC(C)(C)C ; CCOC(=O)CN(CCCCCCCNC(=O)[C@H](N)C1CCCCC1)C(=O)OC(C)(C)C]

### Step 1: Synthesis of 2-3

To a mixture of 1-6 (202.55 mg, 695.25 μmol, 1.1 eq), HOBt (128.11 mg, 948.06 μmol, 1.5 eq) and EDCI (363.49 mg, 1.90 mmol, 3 eq) in DCM (2 mL) was added DIEA (245.06 mg, 1.90 mmol, 330.27 μL, 3 eq) and the mixture was stirred at 20° C. for 5 min. Then, 2-1 (200 mg, 632.04 μmol, 1 eq) was added. The mixture was stirred at 20° C. for 1 hr. The mixture was then poured into a solution of saturated NaHCO₃ (3 mL) and extracted with DCM (3 mL*3). The combined organic layers were dried over anhydrous Na₂SO₄, filtered, and concentrated to give a residue. The residue was purified by flash silica gel chromatography (8 g SepaFlash silica flash column; eluent: 0-50% ethyl acetate/petroleum ether; gradient at 40 mL/min) and the eluent was concentrated. 2-3 (170 mg, 288.25 μmol, 45.61% yield) was obtained as a white solid.

LCMS (Method E): Rt=0.659 min, [M−99]⁺=490.4.

### Step 2: Synthesis of 2-4

To a solution of N-3 (150 mg, 254.34 μmol, 1 eq) in EtOAc (3 mL) was added Pd(OH)₂/C (50 mg, 20% purity) under N₂. The suspension was degassed under vacuum and purged with H₂ several times. The mixture was stirred under H₂ (15 psi) at 20° C. for 12 hours. The mixture was filtered and washed with EA (5 mL*4) and the filtrate was collected. The filtrate was concentrated to give the crude product. 2-4 (100 mg, crude) was obtained as brown oil. LCMS (Method E): Rt=0.325 min, [M+H]⁺=456.3.

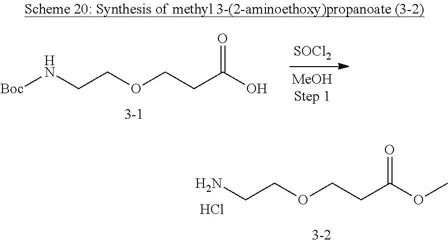

### Step 1: Synthesis of 3-2

To a solution of 3-1 (25 g, 107.18 mmol, 1 eq) in MeOH (250 mL) was added SOCl₂ (15.30 g, 128.61 mmol, 9.34 mL, 1.2 eq) at 0° C. The mixture was stirred at 25° C. for 16 hr. The reaction mixture was filtered and concentrated under reduced pressure to give crude 3-2 (20 g, crude, HCl) as a white solid. ¹H NMR (400 MHz, CDCl₃) δ 8.50-7.72 (m, 3H), 5.20-4.60 (m, 1H), 3.82-3.77 (m, 4H), 3.70 (s, 3H), 3.27 (s, 2H), 2.67-2.63 (m, 2H).

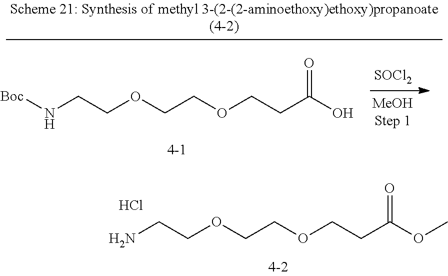

### Step 1: Synthesis of 4-2

To a solution of 4-1 (25 g, 90.15 mmol, 1 eq) in MeOH (250 mL) was added SOCl₂ (12.87 g, 108.18 mmol, 7.86 mL, 1.2 eq) at 0° C. The mixture was stirred at 25° C. for 16 hr. The reaction mixture was filtered and concentrated under reduced pressure to give crude 4-2 (27 g, crude, HCl salt) as a white solid. ¹H NMR (400 MHz, CDCl₃) δ 8.31-7.80 (m, 4H), 4.95 (s, 2H), 3.84-3.81 (m, 2H), 3.78-3.74 (m, 2H), 3.71-3.63 (m, 7H), 3.27 (s, 2H), 2.63-2.59 (m, 2H).

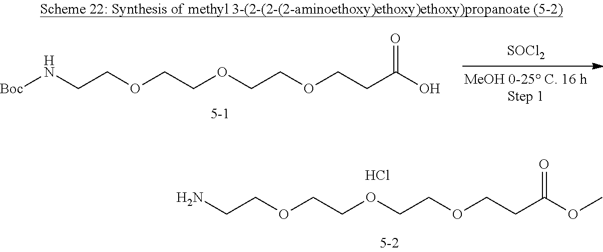 [SMILES: CC(C)(C)OC(=O)NCCOCCOCCOCCC(=O)O ; "SOCl2" ; COC(=O)CCOCCOCCOCCN ; Cl]

### Step 1: Synthesis of 5-2

To a solution of 5-1 (27 g, 84.02 mmol, 1 eq) in MeOH (270 mL) was added SOCl₂ (11.99 g, 100.82 mmol, 7.32 mL, 1.2 eq) at 0° C. The mixture was stirred at 0-25° C. for 16 h. The mixture was concentrated under reduced pressure to give a residue. The crude product was used in the next step without further purification. 5-2 (25 g) was obtained as a colorless oil. ¹H NMR (400 MHz, CDCl₃) δ 8.01 (s, 2H), 3.77 (m, 2H), 3.69 (m, 2H), 3.67-3.59 (m, 7H), 3.57 (s, 4H), 3.26-3.14 (m, 2H), 2.56 (m, 2H).

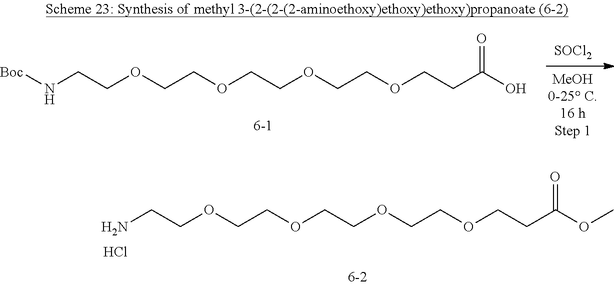 [SMILES: CC(C)(C)OC(=O)NCCOCCOCCOCCOCCC(=O)O ; "SOCl2" ; COC(=O)CCOCCOCCOCCOCCN ; Cl]

### Step 1: Synthesis of 6-2

To a solution of 6-1 (27 g, 73.89 mmol, 1 eq) in MeOH (270 mL) was added SOCl₂ (10.55 g, 88.67 mmol, 6.44 mL, 1.2 eq) at 0° C. The mixture was stirred at 0-25° C. for 16 h. The mixture was concentrated under reduced pressure to give a residue. The crude product was used in the next step without further purification. 6-2 (25.6 g, HCl salt) was obtained as a yellow oil. ¹H NMR (400 MHz, CDCl₃) δ 7.98 (s, 2H), 3.78 (m, 2H), 3.71 (m, 2H), 3.67-3.54 (m, 15H), 3.21-3.20 (d, J=4.4 Hz, 2H), 2.56 (m, 2H).

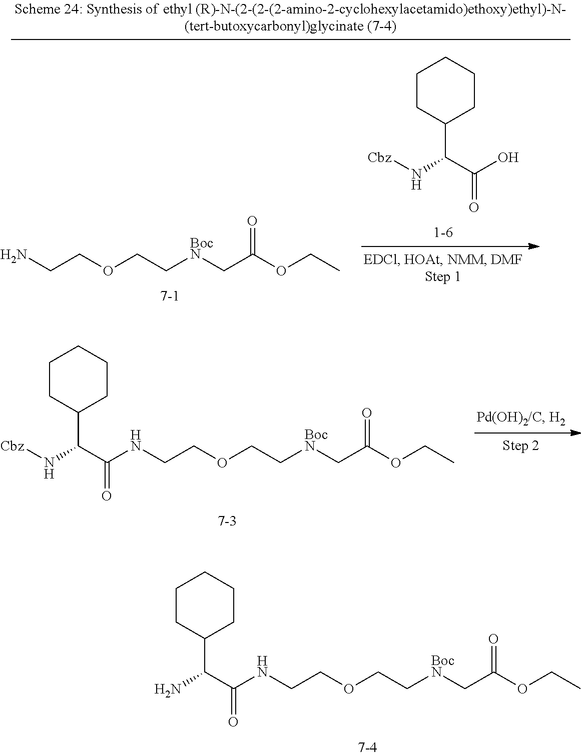 [SMILES: O=C(N[C@@H](C(=O)O)C1CCCCC1)OCc1ccccc1 ; CCOC(=O)CN(CCOCCN)C(=O)OC(C)(C)C ; CCOC(=O)CN(CCOCCNC(=O)[C@H](NC(=O)OCc1ccccc1)C1CCCCC1)C(=O)OC(C)(C)C ; CCOC(=O)CN(CCOCCNC(=O)[C@H](N)C1CCCCC1)C(=O)OC(C)(C)C]

### Step 1: Synthesis of 7-3

To a solution of 1-6 (280 mg, 961.07 μmol, 1 eq), 7-1 (279.05 mg, 961.07 μmol, 1 eq), EDCI (368.48 mg, 1.92 mmol, 2 eq), and HOAt (65.41 mg, 480.54 μmol, 67.22 μL, 0.5 eq) in DCM (3 mL) was added NMM (486.05 mg, 4.81 mmol, 528.31 μL, 5 eq). The mixture was stirred at 25° C. for 1 hr. The reaction mixture was then poured into water (10 mL), extracted with DCM (10 mL*2), and the organic layer was washed with brine (10 mL), dried over Na₂SO₄, and concentrated to afford the crude product. The crude product was purified by flash silica gel column (SiO₂, PE:EA=5:1 to 1:1) to afford 7-3 (270 mg, 478.99 μmol, 49.84% yield, 99% purity) as a colorless oil. LCMS (Method E): Rt=0.612 min, [M+H]⁺=564.5. SFC: Rt=1.904 min, ee=95.1%. ¹H NMR (400 MHz, CDCl₃) δ 7.44-7.30 (m, 5H), 5.11 (s, 2H), 4.29-4.09 (m, 4H), 4.08-3.93 (m, 2H), 3.69-3.35 (m, 8H), 1.87-1.68 (m, 5H), 1.54-1.38 (m, 9H), 1.32-1.24 (m, 6H), 1.14 (br s, 2H).

### Step 2: Synthesis of 7-4

To a solution of 7-3 (270 mg, 478.99 μmol, 1 eq) in MeOH (3 mL) was added Pd(OH)₂/C (200 mg, 284.83 μmol, 20% purity). The mixture was stirred at 25° C. for 16 hr under H₂ (15 psi). The reaction mixture was filtered and the filtrate was concentrated under vacuum to give 7-4 (150 mg, crude) as a colorless oil. LCMS (Method E): Rt=0.448 min, [M+H]⁺=430.6.

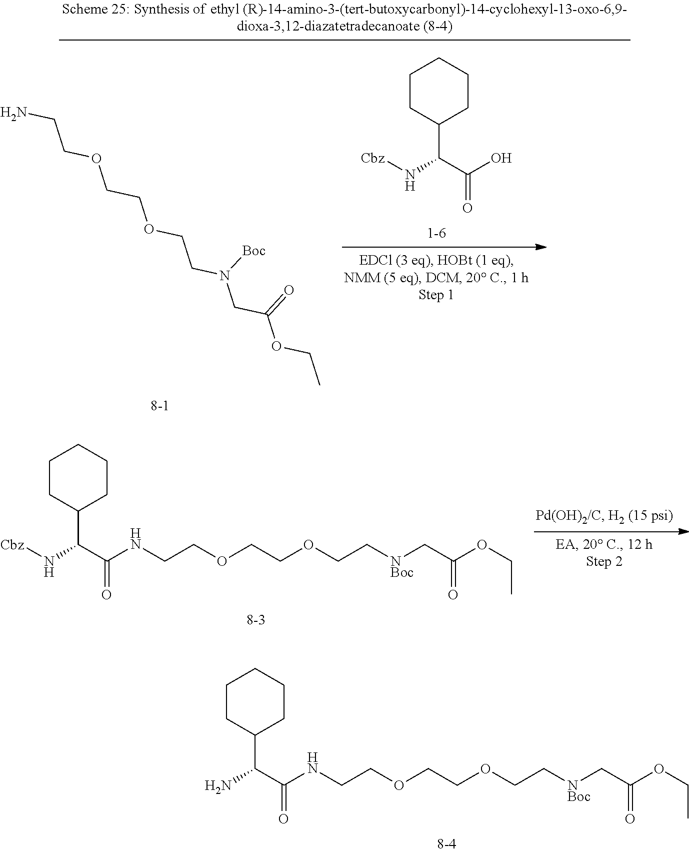 [SMILES: CCOC(=O)CN(CCOCCOCCN)C(=O)OC(C)(C)C ; O=C(N[C@@H](C(=O)O)C1CCCCC1)OCc1ccccc1 ; CCOC(=O)CN(CCOCCOCCNC(=O)[C@H](NC(=O)OCc1ccccc1)C1CCCCC1)C(=O)OC(C)(C)C ; CCOC(=O)CN(CCOCCOCCNC(=O)[C@H](N)C1CCCCC1)C(=O)OC(C)(C)C]

### Step 1: Synthesis of 8-3

To a mixture of 1-6 (287.50 mg, 986.82 μmol, 1.1 eq), EDCI (515.93 mg, 2.69 mmol, 3 eq) and HOBt (121.22 mg, 897.11 μmol, 1 eq) in DCM (3 mL) was added NMM (453.70 mg, 4.49 mmol, 493.15 L, 5 eq) and the mixture was stirred at 20° C. for 10 min. Then, 8-1 (300 mg, 897.11 μmol, 1 eq) was added. The mixture was stirred at 20° C. for 50 min. The mixture was poured into a solution of saturated NaHCO₃ (10 mL) and extracted with DCM (10 mL*3). The combined organic layers were dried over anhydrous Na₂SO₄, filtered, and concentrated to give a residue. The residue was purified by flash silica gel chromatography (40 g SepaFlash silica flash column; eluent: 0-10% MeOH/DCM; gradient at 60 mL/min) and the eluent was concentrated to give the product. The product was repurified by prep-HPLC (column: YMC-Actus Triart C18 150 mm*30 mm*7 um; mobile phase: [water (FA):ACN]; gradient: 63%-93% ACN over 10 min) and concentrated to remove MeCN, followed by adjustment of the pH to 7-8 with a solution of saturated NaHCO₃ and extraction with DCM (10 mL*3). The combined organic layers were dried over anhydrous Na₂SO₄, filtered and concentrated to afford 8-3 (300 mg, 493.64 μmol, 55.03% yield) as an off-white solid. LCMS (Method E): Rt=0.606 min, [M+H]⁺=608.4. SFC: Rt=1.972 min. ¹H NMR (400 MHz, methanol-d₄) δ 7.56-7.25 (m, 5H), 5.08 (s, 2H), 4.24-4.12 (m, 2H), 4.03 (d, J=8.0 Hz, 2H), 3.92 (d, J=6.4 Hz, 1H), 3.61-3.49 (m, 7H), 3.49-3.32 (m, 5H), 1.84-1.59 (m, 6H), 1.55-1.38 (m, 9H), 1.35-0.98 (m, 8H).

### Step 2: Synthesis of 8-4

To a solution of 8-3 (300 mg, 493.64 μmol, 1 eq) in EtOAc (5 mL) was added Pd(OH)₂/C (100 mg, 20% purity) under N₂. The suspension was degassed under vacuum and purged with H₂ several times. The mixture was stirred under H₂ (15 psi) at 20° C. for 12 hours. The mixture was filtered and washed with EA (5 mL*3). The filtrate was concentrated to give 8-4 (200 mg, crude) as a colorless oil. LCMS (Method E): Rt=0.463 min, [M+H]⁺=474.4.

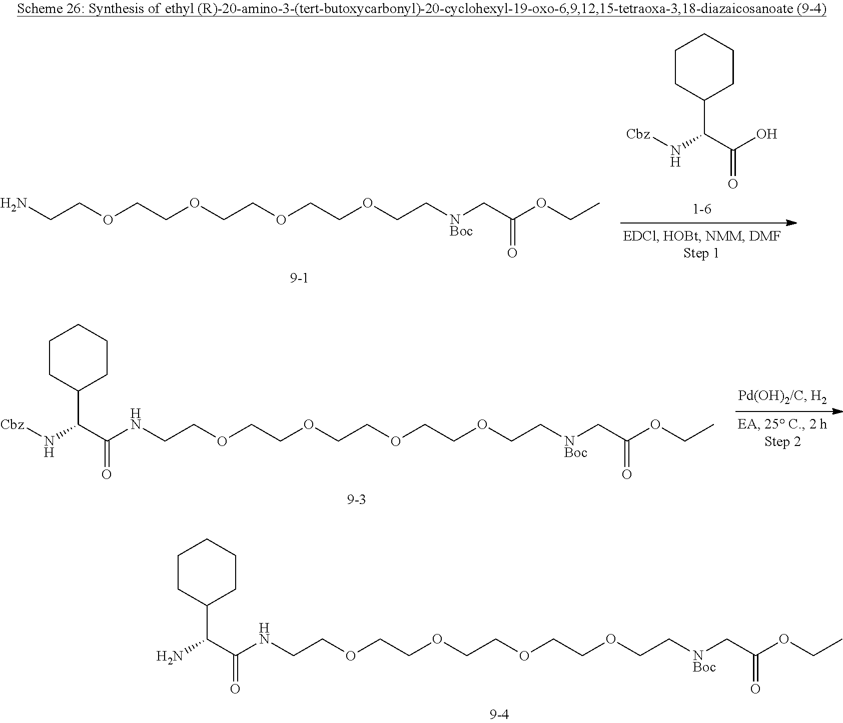 [SMILES: O=C(N[C@@H](C(=O)O)C1CCCCC1)OCc1ccccc1 ; CCOC(=O)CN(CCOCCOCCOCCOCCN)C(=O)OC(C)(C)C ; CCOC(=O)CN(CCOCCOCCOCCOCCNC(=O)[C@H](NC(=O)OCc1ccccc1)C1CCCCC1)C(=O)OC(C)(C)C ; CCOC(=O)CN(CCOCCOCCOCCOCCNC(=O)[C@H](N)C1CCCCC1)C(=O)OC(C)(C)C]

### Step 1: Synthesis of 9-3

To a solution of 9-1 (110 mg, 260.35 μmol, 1 eq) and 1-6 (83.44 mg, 286.38 μmol, 1.1 eq) in DMF (1 mL) was added EDCI (99.82 mg, 520.70 μmol, 2 eq), NMM (131.67 mg, 1.30 mmol, 143.12 μL, 5 eq) and HOBt (17.59 mg, 130.17 μmol, 0.5 eq). The mixture was stirred at 25° C. for 2 hr. The reaction mixture was quenched with water (10 mL). The mixture was extracted with EA (5 mL*3) and dried over anhydrous Na₂SO₄. The mixture was filtered and the filtrate was concentrated under vacuum. The residue was purified by flash silica gel chromatography (20 g SepaFlash silica flash column; eluent: 60-100% ethyl acetate/petroleum ether; gradient at 20 mL/min). 9-3 (150 mg, 215.57 μmol, 82.80% yield, 100% purity) was obtained as a white solid. LCMS (Method E): Rt=0.600 min, [M+H]⁺=696.5. ¹H NMR (400 MHz, DMSO-d₆) δ=8.02-7.96 (m, 1H), 7.40-7.28 (m, 5H), 7.26-7.20 (m, 1H), 5.01 (s, 2H), 4.14-4.08 (m, 2H), 3.95 (d, J=8.4 Hz, 2H), 3.85-3.80 (m, 1H), 3.56-3.34 (m, 18H), 3.31-3.22 (m, 1H), 3.19-3.11 (m, 2H), 1.70-1.47 (m, 6H), 1.43-1.30 (m, 9H), 1.22-1.17 (m, 3H), 1.13-0.92 (m, 4H).

### Step 2: Synthesis of 9-4

To a mixture of Pd(OH)₂/C (50 mg, 50% purity) in EtOAc (2 mL) was added a solution of 9-3 (150 mg, 215.57 μmol, 1 eq) in EtOAc (2 mL) under N₂. The suspension was degassed under vacuum and purged with H₂ several times. The mixture was stirred under H₂ (15 psi) at 25° C. for 2 hours. The reaction mixture was washed by MeOH 15 mL (5 mL*3) and then filtered to give a residue. The residue was used in the next step without further purification. 9-4 (110 mg, 195.83 μmol, 90.84% yield) was obtained as a white solid. ¹H NMR (400 MHz, DMSO-d₆) δ 7.87-7.83 (m, 1H), 4.13-4.07 (m, 2H), 3.94 (d, J=8.4 Hz, 2H), 3.53-3.44 (m, 14H), 3.42-3.38 (m, 2H), 3.36 (s, 2H), 3.27-3.18 (m, 2H), 3.16 (d, J=5.2 Hz, 2H), 2.90 (d, J=5.2 Hz, 1H), 1.69-1.47 (m, 7H), 1.41-1.31 (m, 9H), 1.21-1.16 (m, 3H), 1.14-0.92 (m, 4H).

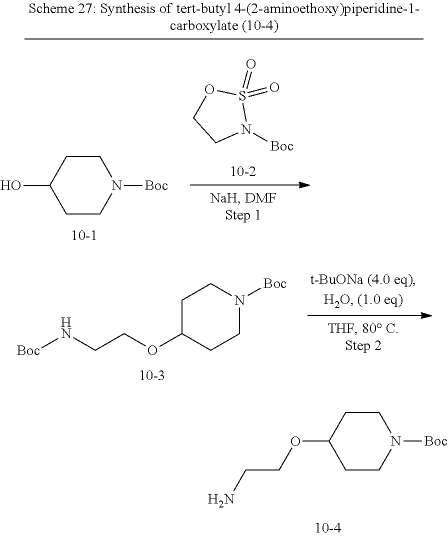 [SMILES: CC(C)(C)OC(=O)N1CCOS1(=O)=O ; CC(C)(C)OC(=O)N1CCC(O)CC1 ; CC(C)(C)OC(=O)NCCOC1CCN(C(=O)OC(C)(C)C)CC1 ; CC(C)(C)OC(=O)N1CCC(OCCN)CC1]

### Step 1: Synthesis of 10-3

To a solution of NaH (4.77 g, 119.25 mmol, 60% purity, 1.2 eq) in DMF (80 mL) was added a solution of 10-1 (20 g, 99.37 mmol, 1 eq) in DMF (120 mL) dropwise at 0° C. The mixture was stirred for 0.5 h at 25° C., and then 10-2 (23.29 g, 104.34 mmol, 1.05 eq) was added to the mixture. The mixture was stirred at 25° C. for 1.5 h. The reaction mixture was quenched with a saturated NH₄Cl solution (200 mL) and extracted with EA (200 mL*2). The organic layer was washed with a citric acid solution (30 mL*3) and dried with anhydrous Na₂SO₄, filtered and concentrated under vacuum. The aqueous phase was adjusted to pH 4 with citric acid and extracted with EA (200 mL*3). The organic layer was dried with anhydrous Na₂SO₄, filtered and concentrated under vacuum. The residue was triturated with PE/EA=20/1 (200 mL), filtered and the filter cake was washed with PE (50 mL*3), the filter cake was dried under vacuum to give 10-3 (31.6 g, 91.74 mmol, 92.32% yield) as white solid. ¹H NMR (400 MHz, CDCl₃) δ 4.87 (br s, 1H), 3.74 (br d, J=6.4 Hz, 2H), 3.62-3.39 (m, 3H), 3.34-3.17 (m, 2H), 3.14-3.00 (m, 2H), 1.80 (br dd, J=2.9, 6.5 Hz, 2H), 1.55-1.36 (m, 20H).

### Step 2: Synthesis of 10-4

To a solution of 10-3 (10 g, 29.03 mmol, 1 eq) in THF (100 mL) was added t-BuONa (11.16 g, 116.13 mmol, 4 eq) and H₂O (523.02 mg, 29.03 mmol, 523.02 μL, 1 eq). The mixture was stirred at 80° C. for 2 hr. The mixture was concentrated under vacuum. The residue was purified by column chromatography (SiO₂, DCM/MeOH=10/1) to give 10-4 (26.5 g, 108.46 mmol, 93.40% yield) as yellow oil. LCMS (Method G): Rt=0.474 min, [M+Na]⁺=267.2. ¹H NMR (400 MHz, DMSO-d₆) δ 3.67-3.54 (m, 2H), 3.49-3.32 (m, 1H), 3.37-3.34 (m, 2H), 3.02 (br d, J=9.2 Hz, 2H), 2.64-2.61 (m, 2H), 1.80-1.72 (m, 2H), 1.46-1.25 (m, 11H).

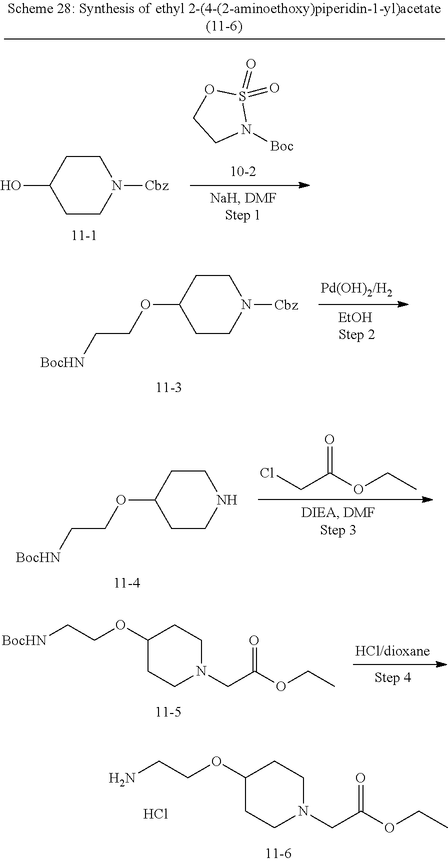

### Step 1: Synthesis of 11-3

In a 500 mL three-neck round-bottomed flask, NaH (18.36 g, 459.03 mmol, 60% purity, 1.2 eq) was dissolved in DMF (500 mL). 11-1 (90 g, 382.53 mmol, 1 eq) in DMF (500 mL) was added dropwise at 0° C. Then the reaction mixture was stirred at 25° C. for 0.5 h. Then 10-2 (89.67 g, 401.65 mmol, 1.05 eq) was added to the above mixture at 0° C. and stirred for another 2.5 h. The reaction mixture was quenched with a saturated NH₄Cl solution (3000 mL) and extracted with MTBE (1500 mL). Then the aqueous phase was extracted with EA (1500 mL*3). The combined organic phase was dried over anhydrous Na₂SO₄, filtered and concentrated to give 11-3 (118 g, 296.20 mmol, 77.43% yield, 95% purity) as yellow oil. ¹H NMR (400 MHz, CDCl₃) δ 7.41-7.29 (m, 5H), 5.13 (s, 2H), 4.86 (br s, 1H), 3.90-3.72 (m, 2H), 3.62-3.43 (m, 3H), 3.30 (br d, J=5.0 Hz, 2H), 3.26-3.17 (m, 2H), 1.82 (br s, 2H), 1.60-1.49 (m, 2H), 1.45 (s, 9H).

### Step 2: Synthesis of 11-4

To a solution of Pd(OH)₂/C (8.02 g, 11.41 mmol, 20% purity, 0.054 eq) in EtOH (800 mL) was added 11-3 (80 g, 211.38 mmol, 1 eq) under N₂. Then the reaction mixture was stirred at 25° C. for 16 hr under H₂ (50 psi) after degassing and refilling with H₂ three times. The reaction mixture was filtered through a pad of celite and concentrated to give 11-4 (58 g, crude) as a yellow oil. LCMS (Method E): Rt=0.534 min, [M+H]⁺+=245.1. ¹H NMR (400 MHz, DMSO-d₆) δ 6.81-6.67 (m, 1H), 3.42-3.32 (m, 3H), 3.09-3.01 (m, 2H), 3.00-2.92 (m, 2H), 2.65-2.55 (m, 2H), 1.90-1.76 (m, 2H), 1.37 (s, 10H).

### Step 3: Synthesis of 11-5

To a solution of 11-4 (30 g, 122.78 mmol, 1 eq) in ACN (300 mL) was added DIEA (31.74 g, 245.57 mmol, 42.77 mL, 2 eq) and ethyl 2-chloroacetate (18.06 g, 147.34 mmol, 15.70 mL, 1.2 eq). Then the reaction mixture was stirred at 40° C. for 16 hr. The reaction mixture was concentrated to afford a residue. The mixture was triturated with EA (200 mL), and the remaining residue was triturated again with EA (100 mL). The precipitate was filtered and the filtrate (desired product) was concentrated. The crude product was diluted with EA (200 mL) and saturated citric acid was added to adjust the pH to 3-4. The mixture was stirred for 0.5 h. The organic phase was separated and the aqueous solution was basified with a NaHCO₃ solution to a pH of 8-9. The aqueous phase was then extracted with DCM (500 mL*2). The combined organic phase was concentrated to give 11-5 (35 g, 105.93 mmol, 86.27% yield) as yellow oil. LCMS (Method E): Rt=0.421 min, [M+H]⁺=331.3. ¹H NMR (400 MHz, DMSO-d₆) δ 6.79-6.63 (m, 1H), 4.13-3.95 (m, 3H), 3.39-3.30 (m, 6H), 3.29-3.20 (m, 1H), 3.16 (s, 2H), 3.09-2.97 (m, 2H), 2.73-2.64 (m, 2H), 2.30-2.18 (m, 2H), 1.99 (s, 1H), 1.85-1.72 (m, 2H), 1.37 (s, 10H), 1.24-1.11 (m, 4H).

### Step 4: Synthesis of 11-6

11-5 (35 g, 105.93 mmol, 1 eq) was dissolved in HCl/dioxane (35 mL). The mixture was stirred at 25° C. for 12 hr. The reaction mixture was concentrated under reduced pressure to remove solvent and give a residue. The residue was triturated with MTBE (500 mL) at 25° C. for 30 min and filtered. The filter cake was dried to give 11-6 (31 g, crude, HCl salt) as a white solid. LCMS (Method B): Rt=0.210 min, [M+H]⁺⁺=231.4. ¹H NMR (400 MHz, D₂O) δ 4.29 (q, J=7.1 Hz, 2H), 4.09 (br s, 2H), 3.89-3.67 (m, 4H), 3.20 (br s, 3H), 3.52-3.15 (m, 3H), 2.20-2.11 (m, 1H), 2.36-2.10 (m, 1H), 2.08-1.75 (m, 1H), 1.81 (br d, J=10.8 Hz, 1H), 1.27 (t, J=7.2 Hz, 3H).

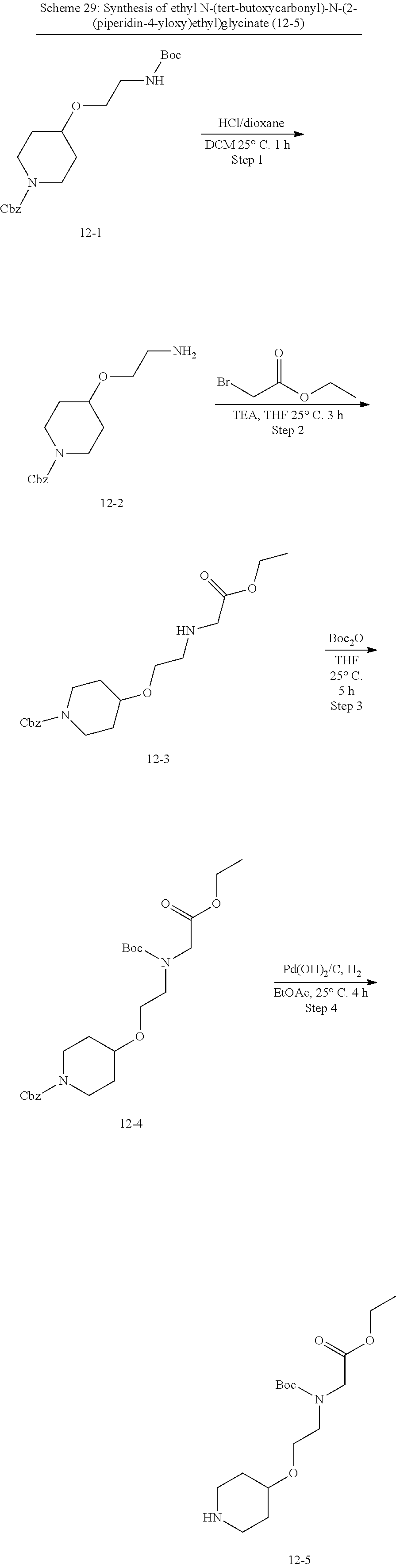

### Step 1: Synthesis of 12-2

To a solution of 12-1 (19 g, 50.20 mmol, 1 eq) in DCM (200 mL) was added HCl/dioxane (4 M in dioxane, 200.00 mL, 15.94 eq). The mixture was stirred at 25° C. for 1 h. The mixture was concentrated under reduced pressure to give a residue. The crude product was used in the next step without further purification. 12-2 (14 g, crude, HCl salt) was obtained as white solid. LCMS (Method E): Rt=0.417 min, [M+H]⁺=297.4.

### Step 2: Synthesis of 12-3

To a solution of 12-2 (12 g, 43.11 mmol, 1 eq) in THF (120 mL) was added TEA (8.72 g, 86.22 mmol, 12.00 mL, 2 eq) and ethyl 2-bromoacetate (7.92 g, 47.42 mmol, 5.25 mL, 1.1 eq). The mixture was stirred at 25° C. for 3 h. The crude product 12-3 (14.1 g, 38.69 mmol, 89.74% yield) was obtained as a colorless oil, which was used in the next step without further purification. LCMS (Method E): Rt=0.423 min, [M+H]⁺=365.2.

### Step 3: Synthesis of 12-4

To a solution of 12-3 (14 g, 38.42 mmol, 1 eq) in THF (140 mL) was added Boc₂O (16.77 g, 76.83 mmol, 17.65 mL, 2 eq). The mixture was stirred at 25° C. for 5 h. The reaction mixture was quenched by addition of water (100 mL) at 25° C., and then extracted with EA (20 mL*3). The organic layer was dried over Na₂SO₄, filtered and concentrated under reduced pressure to give a residue. The residue was purified by flash silica gel chromatography (120 g SepaFlash silica flash column. eluent: 0-80% ethyl acetate/petroleum ether gradient at 100 mL/min). 12-4 (10.5 g, 20.75 mmol, 54.01% yield, 91.8% purity) was obtained as a colorless oil. LCMS: Rt=0.626 min, [M+Na]⁺=487.2.

### Step 4: Synthesis of 12-5

To a solution of Pd(OH)₂/C (50% purity, 1.00 eq) in EtOAc (20 mL) was added 12-4 (10 g, 21.53 mmol, 1 eq) in EtOAc (80 mL) under N₂. The suspension was degassed under vacuum and purged with H₂ several times. The mixture was stirred under H₂ (15 psi) at 25° C. for 4 h. The reaction mixture was filtered to remove Pd(OH)₂/C, and the filtrate was concentrated. The crude product was used in the next step without further purification. 12-5 (28.6 g) was obtained as a colorless oil. ¹H NMR (400 MHz, CDCl₃) δ 4.17 (m, 2H), 4.08-3.97 (m, 2H), 3.63-3.53 (m, 2H), 3.50-3.39 (m, 2H), 3.38-3.28 (m, 1H), 3.11-2.98 (m, 2H), 2.61 (m, 2H), 2.20 (s, 2H), 1.95-1.79 (m, 2H), 1.46-1.36 (m, 9H), 1.29-1.24 (m, 3H).

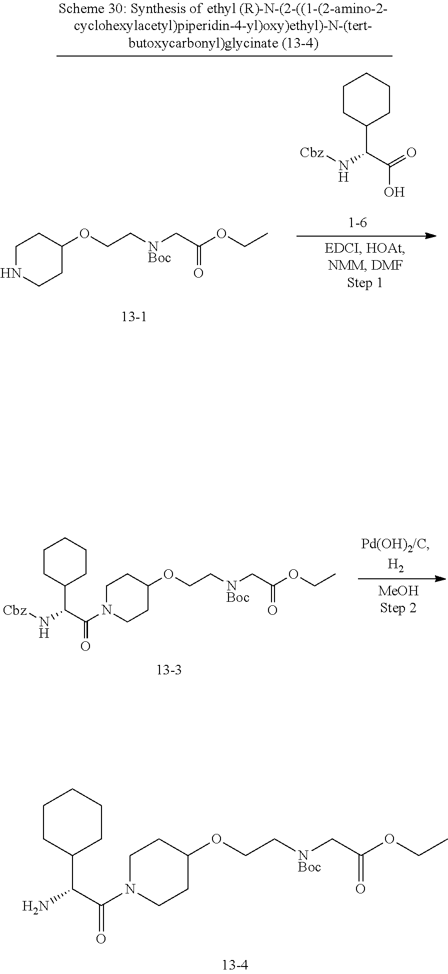 [SMILES: O=C(N[C@@H](C(=O)O)C1CCCCC1)OCc1ccccc1 ; CCOC(=O)CN(CCOC1CCNCC1)C(=O)OC(C)(C)C ; CCOC(=O)CN(CCOC1CCN(C(=O)[C@H](NC(=O)OCc2ccccc2)C2CCCCC2)CC1)C(=O)OC(C)(C)C ; CCOC(=O)CN(CCOC1CCN(C(=O)[C@H](N)C2CCCCC2)CC1)C(=O)OC(C)(C)C]

### Step 1: Synthesis of 13-3

To a solution of 1-6 (283 mg, 858.10 μmol, 1 eq), 13-1 (250 mg, 858.10, 1 eq), EDCI (368.48 mg, 1.92 mmol, 2 eq), and HOAt (65.41 mg, 480.54 μmol, 67.22 μL, 0.5 eq) in DCM (3 mL) was added NMM (486.05 mg, 4.81 mmol, 528.31 μL, 5 eq). The mixture was stirred at 25° C. for 1 hr. The reaction mixture was poured into water (10 mL) and extracted with DCM (10 mL*2). The organic layer was washed with brine (10 mL), dried over Na₂SO₄, and concentrated to afford the crude product. The crude product was purified by flash silica gel column (SiO₂, PE:EA=10:1 to 3:1) to yield 13-3 (400 mg, 653.26 μmol, 76.13% yield, 98.6% purity) as a colorless oil. LCMS (Method E): Rt=0.648 min, [M+H]⁺=603.4. SFC: Rt=1.31 min, ee=94.5%.

### Step 2: Synthesis of 13-4

To a solution of 13-3 (400 mg, 662.53 μmol, 1 eq) in MeOH (4 mL) was added Pd(OH)₂/C (232.61 mg, 331.27 μmol, 20% purity). The mixture was stirred at 25° C. for 16 hr under H₂ (15 psi). The reaction mixture was filtered and the filtrate was concentrated under vacuum to yield 13-4 (300 mg, 611.99 mol, 92.37% yield, 95.8% purity) as a colorless oil, which was used in next step without purification. LCMS (Method E): Rt=0.466 min, [M+H]⁺=470.4.

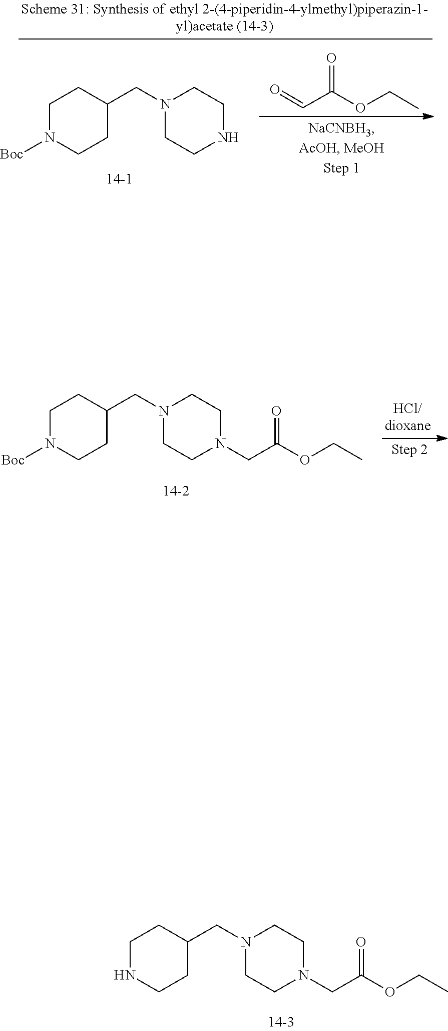

### Step 1: Synthesis of 14-2

To a solution of 14-1 (500 mg, 1.76 mmol, 1 eq) and ethyl 2-oxoacetate (720.43 mg, 7.06 mmol, 2 eq) in MeOH (5 mL) was added CH₃COOH (635.68 mg, 10.59 mmol, 605.98 μL, 6 eq) and the mixture was stirred at 25° C. for 5 hrs. NaBH₃CN (443.47 mg, 7.06 mmol, 4 eq) was added and the mixture was stirred at 25° C. for 10 hrs. The mixture was diluted with water (5 mL) and extracted with DCM (5 mL*3). The combined organic layers were dried over Na₂SO₄, filtered and concentrated in vacuum to give a residue. The residue was purified by column chromatography (SiO₂, PE:EA=5:1 to DCM:MeOH=0:1). The eluted fractions were concentrated in vacuum to give 14-2 (650 mg, 1.58 mmol, 89.74% yield, 90% purity) as a light yellow oil. LCMS: (Method D) Rt=0.398 min, [M+H]⁺=370.4. LCMS: (Method D) Rt=0.279 min, [M+H]⁺=370.4. ¹H NMR (400 MHz, DMSO-d₆) δ=4.13-4.03 (m, 2H), 3.91 (d, J=12.4 Hz, 2H), 3.47-3.33 (m, 4H), 2.97-2.96 (m, 4H), 2.71 (d, J=1.6 Hz, 4H), 1.91 (s, 3H), 1.67 (d, J=12.0 Hz, 2H), 1.39 (s, 9H), 1.21-1.17 (m, 3H), 1.02-1.00 (m, 2H).

### Step 2: Synthesis of 14-3

To a solution of 14-2 (600 mg, 1.62 mmol, 1 eq) in DCM (0.5 mL) was added HCl/dioxane (4 M, 1 mL, 2.46 eq). The mixture was stirred at 25° C. for 0.5 hr. The reaction mixture was concentrated in vacuum to give 14-3 (450 mg, crude, HCl salt) as a white solid which was used in the next step directly without further purification. LCMS (Method A): Rt=0.237 min, [M+H]⁺=270.3.

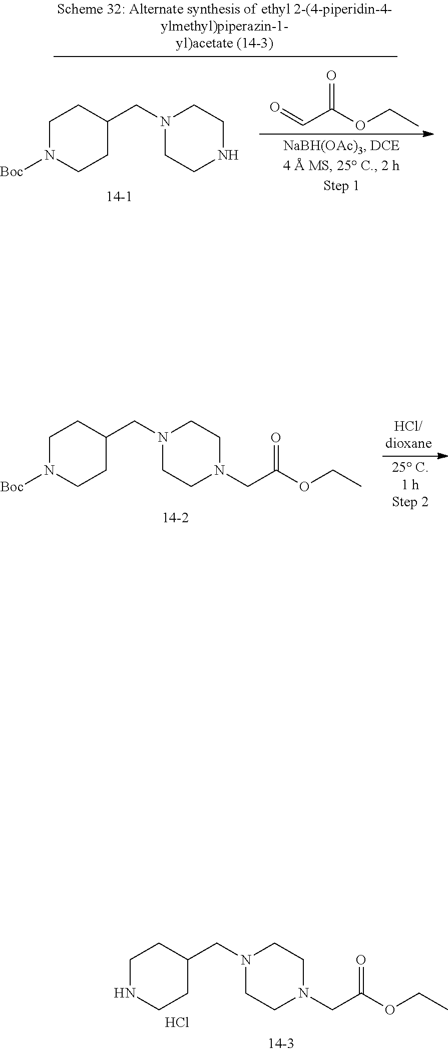

### Step 1 Synthesis of 14-3

To a solution of 14-1 (15 g, 52.93 mmol, 1 eq) in DCE (150 mL) was added 4 Å molecular sieves (1.5 g). The mixture was stirred at 25° C. for 0.25 h, followed by addition of ethyl 2-oxoacetate (16.21 g, 79.39 mmol, 1.5 eq) dropwise at 25° C. The mixture was stirred at 25° C. for 0.75 h then NaBH(OAc)₃ (28.04 g, 132.32 mmol, 2.5 eq) was added. The mixture was stirred at 25° C. for 1 h. The reaction mixture was slowly quenched with H₂O (200 mL) and then extracted with DCM (80 mL*3). The combined organic layers were dried over Na₂SO₄, filtered and concentrated under reduced pressure to give a residue. The residue was purified by flash silica gel chromatography (120 g SepaFlash silica flash column; eluent: 0-10% methanol/ethyl acetate at 100 mL/min). 14-2 (16 g, 43.30 mmol, 81.81% yield) was obtained as a yellow solid. ¹H NMR (400 MHz, DMSO-d₆) δ 4.09-4.04 (m, 2H), 3.91-3.89 (d, J=11.6 Hz, 2H), 3.17 (s, 2H), 2.75-2.56 (m, 2H), 2.49-2.45 (m, 2H), 2.34 (s, 4H), 2.11-2.09 (d, J=6.8 Hz, 2H), 1.90 (s, 2H), 1.70-1.57 (m, 3H), 1.38 (s, 9H), 1.19-1.12 (m, 3H), 0.98-0.82 (m, 2H).

### Step 2: Synthesis of 14-3

To a solution of 14-2 (30 g, 81.19 mmol, 1 eq) in DCM (300 mL) was added HCl/dioxane (4 M, 300 mL, 14.78 eq). The mixture was stirred at 25° C. for 1 h. The reaction mixture was concentrated under reduced pressure to give a residue. The residue was used in the next step without further purification. 14-3 (24 g, 78.47 mmol, 96.65% yield, HCl salt) was obtained as a white solid. ¹H NMR (400 MHz, METHANOL-d4) δ 4.42-4.28 (m, 4H), 3.99-3.74 (m, 7H), 3.66 (s, 1H), 3.46-3.43 (d, J=12.8 Hz, 2H), 3.34-3.32 (d, J=6.8 Hz, 2H), 3.08 (m, 2H), 2.34 (m, 1H), 2.23-2.20 (d, J=14.4 Hz, 2H), 1.67-1.52 (m, 2H), 1.34 (m, 3H).

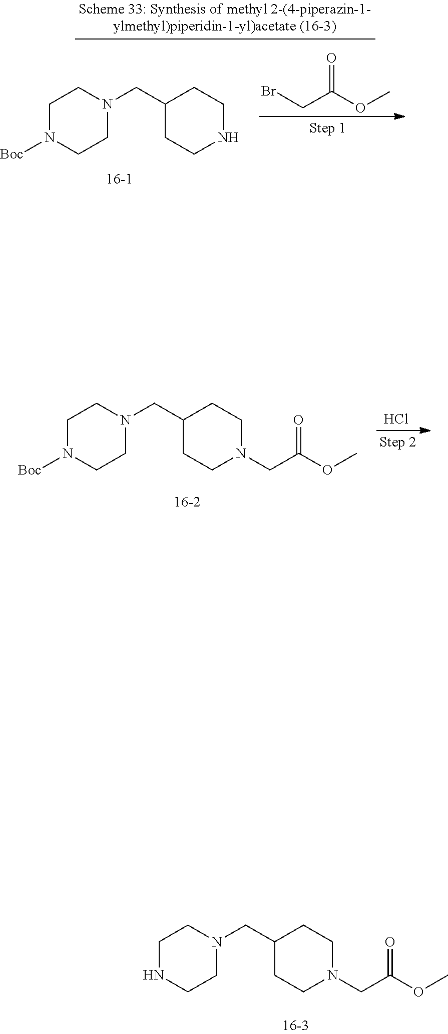

### Step 1: Synthesis of 16-2

To a solution of 16-1 (0.3 g, 1.06 mmol, 1 eq) and methyl 2-bromoacetate (178.12 mg, 1.16 mmol, 110.22 μL, 1.1 eq) in ACN (3 mL) was added DIEA (273.61 mg, 2.12 mmol, 368.75 μL, 2 eq) and the mixture was stirred at 40° C. for 1 hr. The mixture was then poured into water (1 mL) and extracted with DCM (1 mL*3) and the organic layers were dried over Na₂SO₄, filtered and concentrated to give 16-2 (0.37 g, 1.04 mmol, 98.33% yield) as a yellow solid. ¹H NMR (400 MHz, CDCl₃) δ 3.72 (s, 3H), 3.43 (s, 4H), 3.23 (s, 2H), 2.94 (d, J=11.6 Hz, 2H), 2.36 (s, 4H), 2.19 (d, J=10.4 Hz, 3H), 2.01 (s, 2H), 1.76 (d, J=12.4 Hz, 2H), 1.46 (s, 9H), 1-8-1.27 (m, 2H).

### Step 2: Synthesis of 16-3

A solution 16-2 (0.37 g, 1.04 mmol, 1 eq) in HCl/dioxane (4 M, 4 mL, 15.37 eq) was stirred at 25° C. for 0.5 hr. The mixture was concentrated to give 16-3 (0.3 g, 1.03 mmol, 98.77% yield, HCl salt) as a white solid. ¹H NMR (400 MHz, methanol-d₄) δ 3.-8-3.84 (m, 3H), 3.71 (s, 4H), 3.66 (d, J=1.6 Hz, 2H), 3.-6-3.33 (m, 2H), 3.-0-3.15 (m, 4H), 2.-4-1.80 (m, 4H), 1-6-1.67 (m, 1H), 1.38 (t, J=4.8 Hz, 4H).

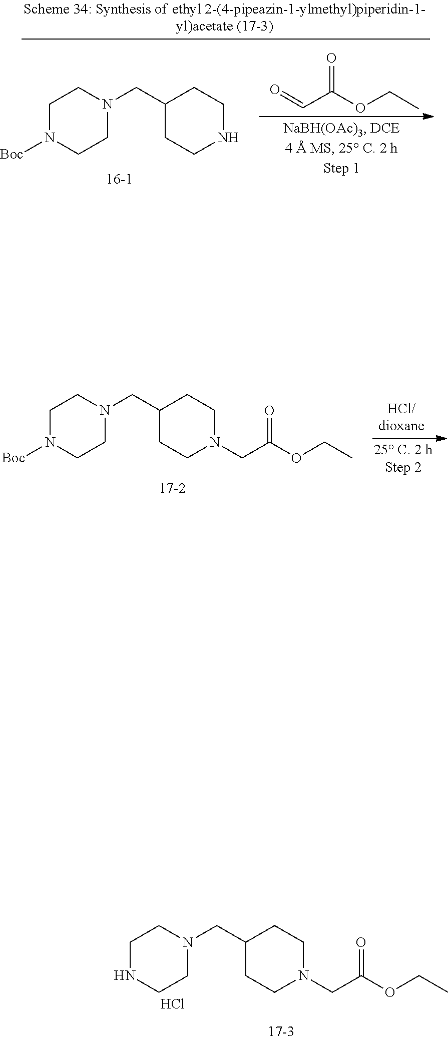

### Step 1: Synthesis of 17-2

To a solution of 16-1 (27 g, 95.27 mmol, 1 eq) in DCE (300 mL) was added 4 Å molecular sieves (2.7 g, 95.27 mmol, 1 eq). The mixture was stirred at 25° C. for 0.25 h, and then ethyl 2-oxoacetate (29.18 g, 142.90 mmol, 1.5 eq) was added dropwise at 25 C. Then the mixture was stirred at 25° C. for another 0.75 h. And NaBH(OAc)3 (50.48 g, 238.17 mmol, 2.5 eq) was added. The mixture was stirred at 25° C. for 1 h. The reaction mixture was quenched slowly by addition of H₂O (600 mL), and then diluted with DCM (150 mL) and extracted with DCM (100 mL*3). The combined organic layers were dried over Na₂SO₄, filtered and concentrated under reduced pressure to give a residue. The residue was purified by flash silica gel chromatography (330 g SepaFlash silica flash column; eluent: 0-10% Methanol/Ethyl acetate at 100 mL/min). 17-2 (33 g, 89.31 mmol, 93.75% yield) was obtained as colorless oil. ¹HNMR (400 MHz, CDCl₃) δ 4.19-4.14 (m, 2H), 3.51-3.38 (m, 5H), 3.27 (s, 2H), 3.03-3.00 (d, J=11.6 Hz, 2H), 2.43 (m, 4H), 2.32-2.24 (m, 4H), 2.01 (s, 2H), 1.75-1.72 (d, J=12.68 Hz, 2H), 1.43 (s, 9H), 1.30-1.26 (m, 3H).

### Step 2: Synthesis of 17-3

To a solution of 17-2 (32 g, 86.60 mmol, 1 eq) in DCM (300 mL) was added HCl/dioxane (4 M in dioxane, 21.65 mL, 1 eq). The mixture was stirred at 25° C. for 2 h. The reaction mixture was concentrated under reduced pressure to give a residue. The residue was used in the next step without further purification. 17-3 (28.3 g, 98.16% yield, HCl salt) was obtained as a white solid. ¹H NMR (400 MHz, methanol-d₄) δ 4.33 (m, 2H), 4.19 (s, 2H), 3.75 (d, J=3.6 Hz, 8H), 3.62-3.37 (m, 2H), 3.32 (m, 2H), 3.23 (m, 2H), 2.46-2.22 (m, 3H), 1.82-1.65 (m, 2H), 1.34 (m, 3H).

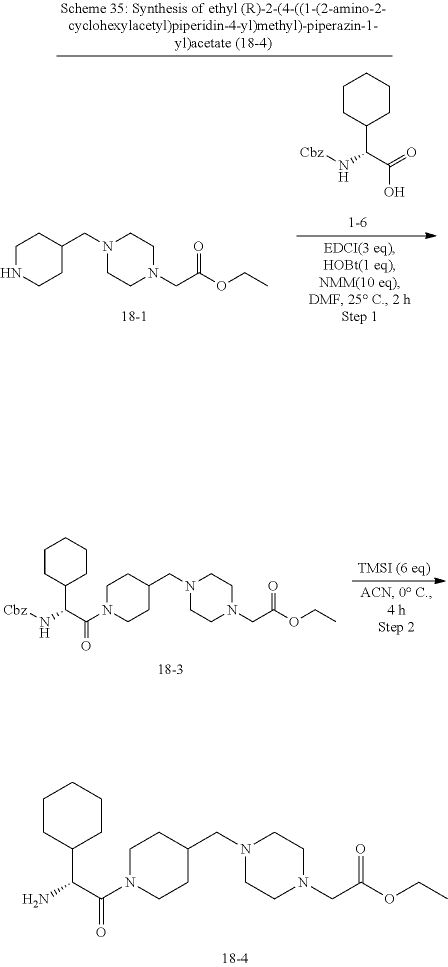 [SMILES: O=C(N[C@@H](C(=O)O)C1CCCCC1)OCc1ccccc1 ; CCOC(=O)CN1CCN(CC2CCNCC2)CC1 ; CCOC(=O)CN1CCN(CC2CCN(C(=O)[C@H](NC(=O)OCc3ccccc3)C3CCCCC3)CC2)CC1 ; CCOC(=O)CN1CCN(CC2CCN(C(=O)[C@H](N)C3CCCCC3)CC2)CC1]

### Step 1: Synthesis of 18-3

To a solution of 18-1 (629.86 mg, 2.06 mmol, 1.2 eq, HCl salt) and 1-6 (500 mg, 1.72 mmol, 1 eq) in DMF (5 mL) was added EDCI (987.00 mg, 5.15 mmol, 3 eq), HOBt (231.90 mg, 1.72 mmol, 1 eq) and NMM (1.74 g, 17.16 mmol, 1.89 mL, 10 eq). The mixture was stirred at 25° C. for 2 h. The reaction mixture was quenched by addition H₂O (1 mL), diluted with EA (1 mL) and extracted with EA (2 mL*3). The combined organic layers were washed with brine (2 mL*3), dried over anhydrous Na₂SO₄, filtered and concentrated under reduced pressure to give a residue. The residue was used in the next step without further purification. 18-3 (800 mg, 1.42 mmol, 82.46% yield, 96.1% purity) was obtained as a yellow gum. LCMS (Method E): Rt=0.470 min, [M+H]⁺=543.4. SFC: Rt=1.184 min, ee>99%.

### Step 2: Synthesis of 18-4

To a solution of 18-3 (800 mg, 1.47 mmol, 1 eq) in ACN (24 mL) was added TMSI (1.77 g, 8.84 mmol, 1.20 mL, 6 eq). The mixture was stirred at 0° C. for 4 h. The reaction mixture was quenched by addition of H₂O (10 mL), diluted with EA (20 mL) and extracted with EA (20 mL*3). The combined organic layers were adjusted to pH 7-8 with a saturated solution of NaHCO₃ and extracted with CHCl₃:i-PrOH (3:1, 10 mL*3), dried over anhydrous Na₂SO₄, filtered and concentrated under reduced pressure to give a residue. The residue was used in the next step without further purification. 18-4 (550 mg, 1.29 mmol, 87.67% yield, 96.9% purity) was obtained as a yellow gum. LCMS (Method E): Rt=0.327 min, [M+H]⁺=409.2. SFC: Rt=2.420 min, ee=93.8%. ¹H NMR (400 MHz, DMSO-d₆) δ 4.39 (d, J=12.0 Hz, 1H), 4.19-3.98 (m, 3H), 3.89 (d, J=13.2 Hz, 1H), 3.46-3.22 (m, 2H), 3.10 (d, J=6.0 Hz, 2H), 3.02-2.90 (m, 1H), 2.62-2.51 (m, 2H), 2.50-2.40 (m, 3H), 2.33 (s, 3H), 2.11-2.08 (m, 2H), 1.81-1.63 (m, 6H), 1.62-1.55 (m, 1H), 1.48 (d, J=8.4 Hz, 1H), 1.30 (d, J=1.6 Hz, 1H), 1.20-1.17 (m, 3H), 1.16-1.06 (m, 3H), 1.05-0.75 (m, 3H).

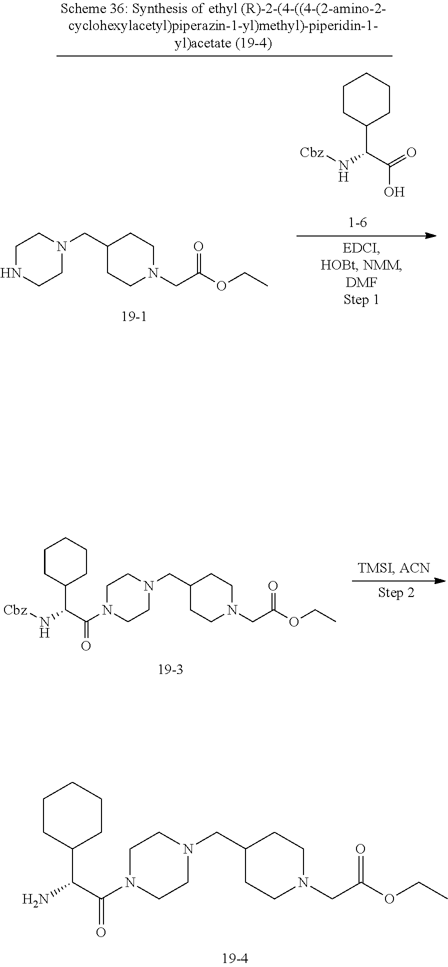 [SMILES: O=C(N[C@@H](C(=O)O)C1CCCCC1)OCc1ccccc1 ; CCOC(=O)CN1CCC(CN2CCNCC2)CC1 ; CCOC(=O)CN1CCC(CN2CCN(C(=O)[C@H](NC(=O)OCc3ccccc3)C3CCCCC3)CC2)CC1 ; CCOC(=O)CN1CCC(CN2CCN(C(=O)[C@H](N)C3CCCCC3)CC2)CC1]

### Step 1: Synthesis of 19-3

To a solution of 19-1 (503.89 mg, 1.65 mmol, 1.2 eq) and 1-6 (400 mg, 1.37 mmol, 1 eq) in DMF (5 mL) was added EDCI (789.60 mg, 4.12 mmol, 3 eq), HOBt (185.52 mg, 1.37 mmol, 1 eq) and NMM (1.39 g, 13.73 mmol, 1.51 mL, 10 eq). The mixture was stirred at 25° C. for 1 hr. The mixture was poured into water (10 mL) and extracted with EA (10 mL*3). The combined organic layers were concentrated to give a residue. The residue was purified by flash silica gel chromatography (12 g SepaFlash silica flash column, eluent: 0-100% ethyl acetate/petroleum ether; gradient at 20 mL/min) and the eluent was concentrated to give 19-3 (300 mg, 542.06 μmol, 39.48% yield, 98.06% purity) as a yellow solid. LCMS (Method E): Rt=0.427 min, [M+H]⁺=543.3. SFC: Rt=2.199 min.

### Step 2: Synthesis of 19-4

To a solution of 19-3 (250 mg, 460.65 μmol, 1 eq) in ACN (3 mL) was added TMSI (553.04 mg, 2.76 mmol, 376.22 μL, 6 eq) at 0° C. The mixture was stirred at 0° C. for 4 hr. The reaction mixture was quenched by addition of H₂O (10 mL), diluted with EA (20 mL), and extracted with EA (20 mL*3). The combined organic layers were adjusted to pH 7-8 with a saturated solution of NaHCO₃, extracted with CHCl₃:i-PrOH (3:1, 10 mL*3), dried over anhydrous Na₂SO₄, filtered and concentrated under reduced pressure to give 19-4 (150 mg, 304.72 μmol, 66.15% yield, 83% purity) as a white solid. LCMS (Method E): Rt=0.317 min, [M+H]⁺=409.4.

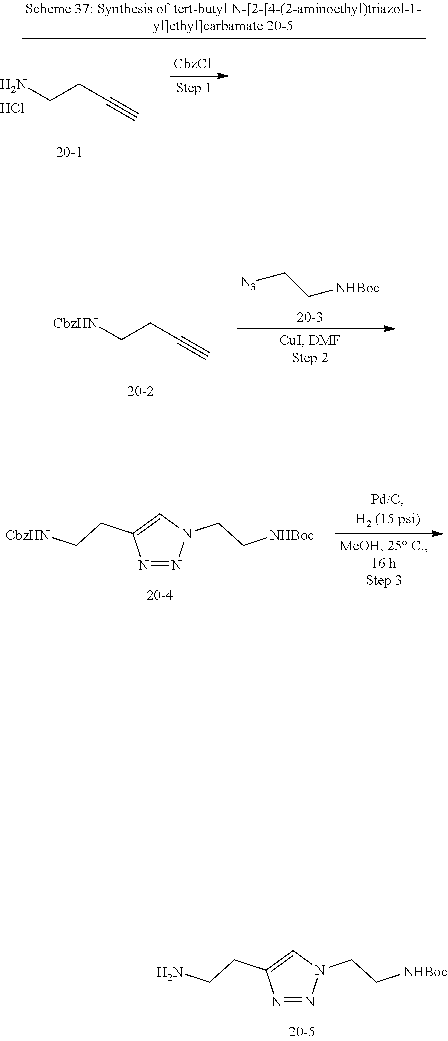

### Step 1: Synthesis of 20-2

To a solution of CbzCl (48.48 g, 284.18 mmol, 40.57 mL, 1 eq) in EtOH (480 mL) and H₂O (480 mL) was added NaHCO₃ (23.87 g, 284.18 mmol, 11.06 mL, 1 eq). Then 20-1 (30 g, 284.18 mmol, 1 eq, HCl) was added at 0° C. The mixture was stirred at 25° C. for 16 hr. The reaction mixture was quenched with water (500 mL). The mixture was extracted with EA (200 mL*3) and dried over anhydrous Na₂SO₄. The mixture was filtered and the filtrate was concentrated under vacuum. The residue was purified by flash silica gel chromatography (220 g SepaFlash silica flash column; eluent: 0-30% ethyl acetate/petroleum ether gradient at 120 mL/min) to give 20-2 (36 g, 132.85 mmol, 46.75% yield, 75% purity) as an off-white oil. LCMS (Method E): Rt=0.485 min, M+H=204.2. ¹H NMR (400 MHz, CDCl₃) δ 7.38-7.35 (m, 5H), 5.11 (s, 2H), 3.42-3.27 (m, 2H), 2.47-2.34 (m, 2H), 2.11-1.99 (m, 1H).

### Step 2: Synthesis of 20-4

To a solution of 20-2 (36 g, 177.13 mmol, 1 eq) and 20-3 (32.98 g, 177.13 mmol, 1 eq) in DMF (300 mL) was added CuI (5.06 g, 26.57 mmol, 0.15 eq). The mixture was stirred at 80° C. for 16 hr. The reaction mixture was filtered through a pad of celite and the celite was washed with ethyl acetate. Then the mixture was diluted with H₂O (1500 mL) and extracted with EA (300 mL*3). The combined organic layers were washed with brine (200 mL*2), dried over anhydrous Na₂SO₄, filtered and concentrated under reduced pressure to give a residue. The residue was purified by flash silica gel chromatography (330 g SepaFlash silica flash column; eluent: 50-100% ethyl acetate/petroleum ether gradient at 150 mL/min). 20-4 (34 g, 82.94 mmol, 46.82% yield, 95% purity) was obtained as a white solid. LCMS (Method E): Rt=0.490 min, [M+H]⁺=390.2. ¹H NMR (400 MHz, CDCl₃) δ 7.39-7.28 (m, 6H), 5.36 (s, 1H), 5.08 (s, 2H), 4.98 (s, 1H), 4.43-4.39 (m, 2H), 3.65-3.46 (m, 4H), 2.94-2.91 (m, 2H), 1.42 (s, 9H).

### Step 3: Synthesis of 20-5

To a solution of 20-4 (20 g, 51.35 mmol, 1 eq) in MeOH (100 mL) was added Pd/C (2.00 g, 1.88 mmol, 10% purity, 0.037 eq) under an N₂ atmosphere. The suspension was degassed and purged with H₂ 3 times. The mixture was stirred under H₂ (15 psi) at 25° C. for 16 hr. The reaction mixture was washed by MeOH 150 mL (50 mL*3) and then filtered to give a residue. The residue was used in the next step without further purification. 20-5 (13 g, 50.92 mmol, 99.15% yield) was obtained as an off-white oil. LCMS (Method E): Rt=0.313 min, [M+H]⁺=256.2. ¹H NMR (400 MHz, CDCl₃) δ 7.39 (s, 1H), 5.31 (s, 1H), 4.43-4.39 (m, 2H), 3.60-3.55 (m, 2H), 3.07-2.95 (m, 2H), 2.84-2.79 (m, 2H), 1.39 (s, 9H).

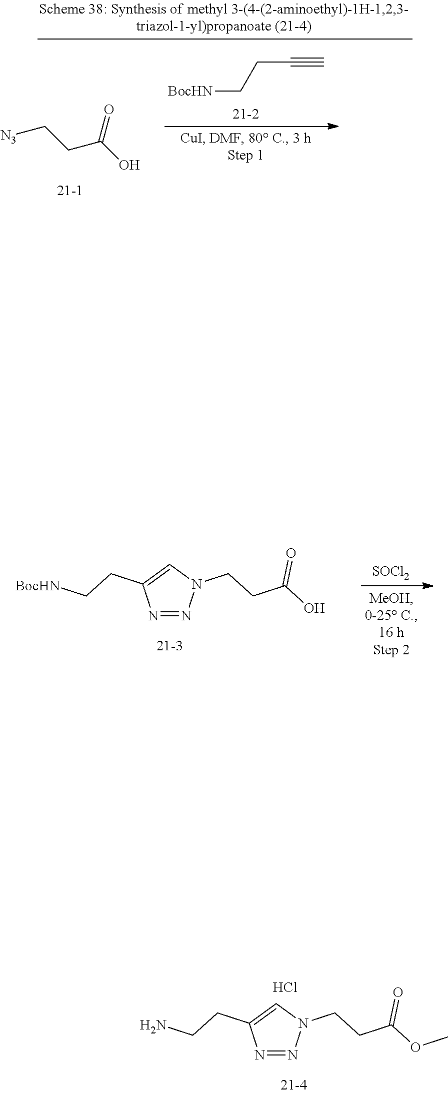

### Step 1: Synthesis of 21-3

To a solution of 21-1 (25 g, 217.22 mmol, 1 eq) in DMF (250 mL) was added CuI (4.14 g, 21.72 mmol, 0.1 eq) and 21-2 (36.76 g, 217.22 mmol, 1 eq). The mixture was stirred at 80° C. for 3 h. The reaction mixture was quenched by addition of water (500 mL) at 25° C., and then extracted with EA (100 mL*3). The organic phase was dried over Na₂SO₄, filtered and concentrated under reduced pressure to give a residue. The residue was purified by flash silica gel chromatography (330 g SepaFlash silica flash column; eluent: 0-100% ethyl acetate/petroleum ether gradient at 100 mL/min). 21-3 (42 g, 147.73 mmol, 68.01% yield) was obtained as yellow solid. LCMS (Method E): Rt=0.389 min, [M+H]⁺=285.2. ¹H NMR (400 MHz, CDCl₃) δ 9.76-8.99 (m, 1H), 7.52 (s, 1H), 5.22 (s, 1H), 4.59 (m, 2H), 3.46-3.23 (m, 2H), 2.93-2.89 (m, 2H), 2.83 (s, 2H), 1.37 (s, 9H).

### Step 2: Synthesis of 21-4

To a solution of 21-3 (40 g, 140.69 mmol, 1 eq) in MeOH (400 mL) was added SOCl₂ (20.09 g, 168.83 mmol, 12.26 mL, 1.2 eq) at 0° C. The mixture was stirred at 25° C. for 16 h. The mixture was concentrated under reduced pressure to give a residue. The crude product was used in the next step without further purification. 21-4 (32 g, 133.63 mmol, 94.98% yield, 98% purity, HCl salt) was obtained as a yellow solid. LCMS (Method E): Rt=0.17 min, [M+H]⁺=199.30. ¹H NMR (400 MHz, METHANOL-d4) δ 8.57 (s, 1H), 4.88 (m, 2H), 3.71 (s, 3H), 3.39-3.37 (d, J=6.4 Hz, 2H), 3.32 (m, 2H), 3.15 (m, 2H).

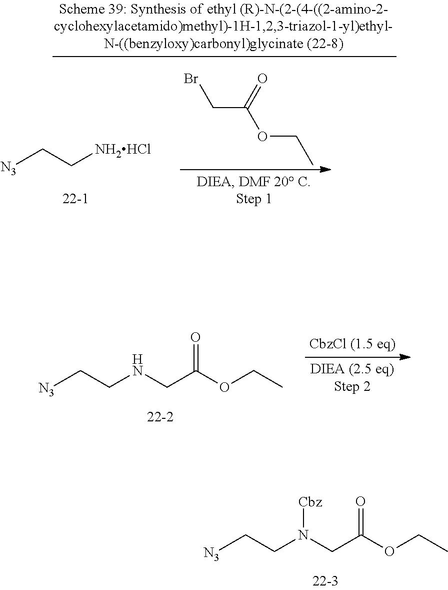

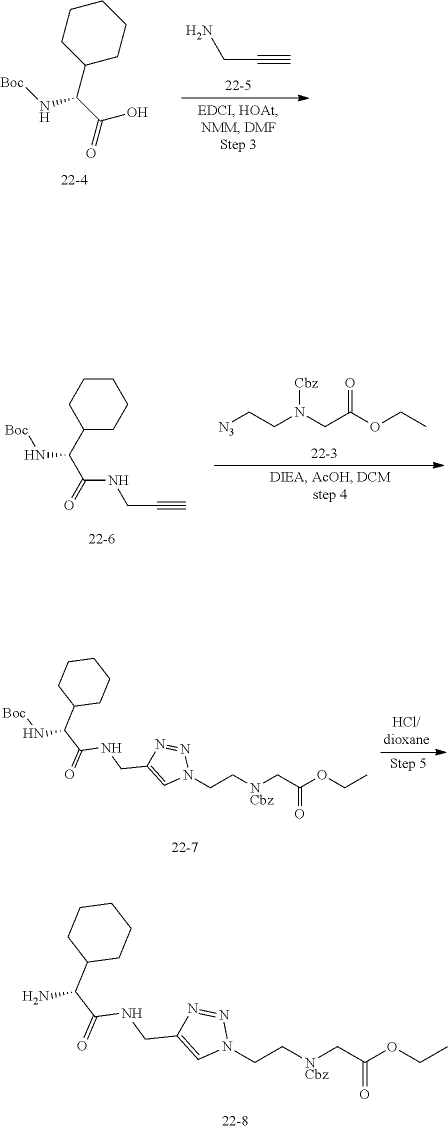

### Step 1: Synthesis of 22-2

To a solution of 22-1 (575 mg, 4.69 mmol, 1 eq) and ethyl 2-bromoacetate (470.11 mg, 2.82 mmol, 311.54 μL, 0.6 eq) in DMF (6 mL) was added TEA (569.71 mg, 5.63 mmol, 783.64 μL, 1.2 eq). The mixture was stirred at 25° C. for 4 hr. The reaction mixture was poured into water (5 ml) and extracted with EA (5 ml*2), and the organic layers were combined and concentrated in vacuum to afford the crude product. The residue was purified by flash silica gel chromatography to afford 22-2 (410 mg, 2.38 mmol, 50.7% yield) as a light-yellow oil. ¹H NMR (400 MHz, CDCl₃) δ 4.28-4.02 (m, 2H), 3.49-3.34 (m, 4H), 2.95-2.86 (m, 1H), 2.83-2.80 (m, 1H), 1.31-1.22 (m, 3H).

### Step 2: Synthesis of 22-3

To a solution of 22-2 (390 mg, 2.27 mmol, 1 eq) in DCM (4 mL) was added CbzCl (579.58 mg, 3.40 mmol, 485.01 μL, 1.5 eq) and DIEA (731.83 mg, 5.66 mmol, 986.29 μL, 2.5 eq) at 0° C. The mixture was stirred at 25° C. for 1 hr. The reaction was poured into water (5 ml) and extracted with DCM (5 ml*3), and then the organic layer was separated and concentrated under vacuum to give the crude product. The residue was purified by flash silica gel chromatography to give 22-3 (500 mg, 1.52 mmol, 67.02% yield, 93% purity) as a yellow oil. LCMS (Method E): Rt=0.539 min, [M+Na)]⁺=329.1. ¹H NMR (400 MHz, CDCl₃) δ 7.41-7.29 (m, 5H), 5.17 (d, J=18.4 Hz, 2H), 4.26-4.03 (m, 4H), 3.59-3.42 (m, 4H), 1.33-1.15 (m, 3H).

### Step 3: Synthesis of 22-6

To a solution of 22-4 (3.59 g, 13.95 mmol, 1 eq) and 22-5 (1.15 g, 20.93 mmol, 1.34 mL, 1.5 eq) in DMF (20 mL) was added EDCI (8.02 g, 41.85 mmol, 3 eq), HOAt (2.85 g, 20.93 mmol, 2.93 mL, 1.5 eq) and NMM (14.11 g, 139.51 mmol, 15.34 mL, 10 eq). The mixture was stirred at 25° C. for 2 hr. The reaction was poured into water (25 ml) and extracted with EA (25 ml*3), and then the organic layer was separated and concentrated under vacuum to give the crude product. The crude product was purified by recrystallization from DCM (50 mL) to give 22-6 (2.0 g, 5.98 mmol, 42.85% yield, 88% purity) as a white solid. LCMS (Method E): Rt=0.517 min, [M+H-56]⁺=239.2. LCMS (Method E): Rt=0.518 min, [M+H-56]⁺=239.2. ¹H NMR (400 MHz, methanol-d₄) δ 4.13-3.99 (m, 1H), 3.93-3.84 (m, 2H), 2.59 (d, J=2.0 Hz, 1H), 1.75 (d, J=8.8 Hz, 3H), 1.69-1.58 (m, 3H), 1.44 (s, 9H), 1.31-0.97 (m, 6H).

### Step 4: Synthesis of 22-7

To a solution of 22-6 (100 mg, 339.69 μmol, 2 eq) and 22-3 (52.03 mg, 169.84 μmol, 1 eq) in DCM (1 mL) was added DIEA (15.37 mg, 118.89 μmol, 20.71 μL, 0.7 eq), CuI (6.47 mg, 33.97 μmol, 0.2 eq) and AcOH (7.14 mg, 118.89 μmol, 6.81 μL, 0.7 eq) at 0° C. The mixture was stirred at 25° C. for 12 hr. The reaction was poured into water (3 ml) and extracted with EA (3 ml*2), and then the organic layer was separated and concentrated under vacuum to give the crude product. The residue was purified by flash silica gel chromatography to give 22-7 (80 mg, 123.85 μmol, 72.92% yield, 93% purity) as a yellow oil. LCMS (Method E): Rt=0.576 min, [M+H]⁺=601.4. SFC: Rt=1.237 min; ee>99%.

### Step 5: Synthesis of 22-8

A solution of 22-7 (50 mg, 83.24 μmol, 1 eq) in HCl/dioxane (0.5 mL) was stirred at 25° C. for 1 hr. The reaction was concentrated under vacuum to give 22-8 (40 mg, 65.54 μmol, 78.74% yield, 88% purity, HCl salt), which was used in the next step without further purification. LCMS (Method E): Rt=0.442 min, [M+H]⁺=501.4.

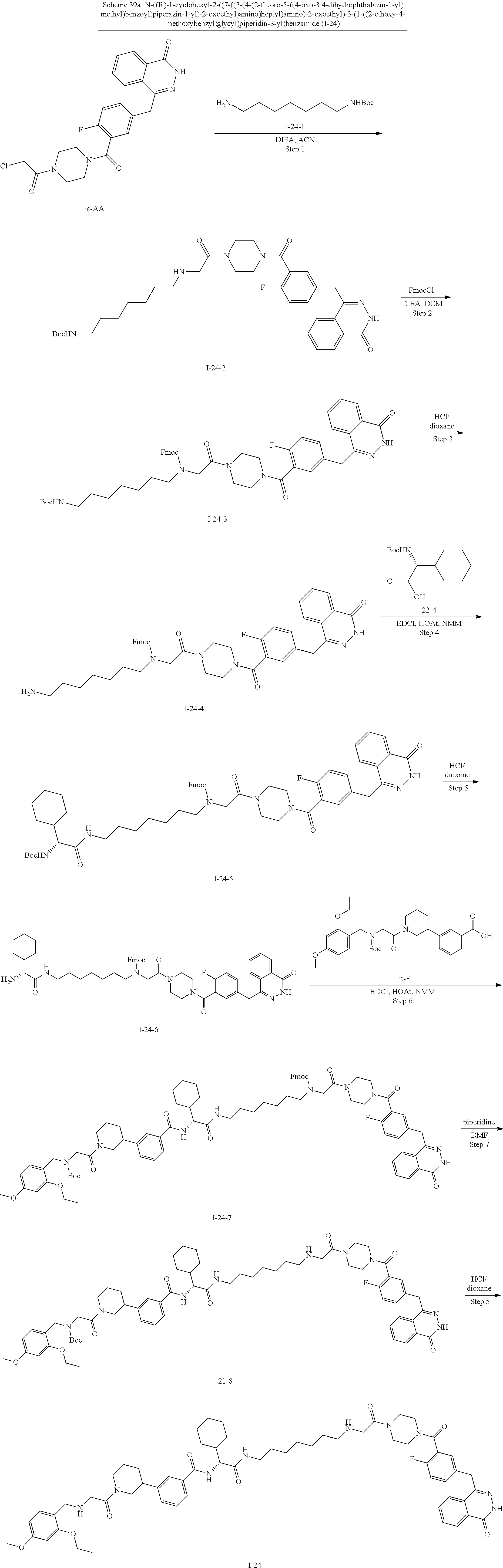

### Step 1: Synthesis of I-24-2

To a solution of Int-AA (0.3 g, 677.40 μmol, 1 eq) and I-24-1 (187.24 mg, 812.88 μmol, 1.2 eq) in ACN (3 mL) was added DIEA (350.20 mg, 2.71 mmol, 471.96 μL, 4 eq) and the mixture was stirred at 40° C. for 4 hr. The mixture was filtered and concentrated to give a residue. The residue was purified by column chromatography (SiO₂, DCM/MeOH=100/1 to 1/100), and the eluent was concentrated to give I-24-2 (0.34 g, 496.31 μmol, 73.27% yield, 92.95% purity) as a yellow solid. LCMS (Method D): Rt=0.320 min, [M+H]⁺=637.4.

### Step 2: Synthesis of I-24-3

To a solution of I-24-2 (0.34 g, 533.96 μmol, 1 eq) in DCM (3.5 mL) was added DIEA (207.03 mg, 1.60 mmol, 279.02 μL, 3 eq) and Fmoc-Cl (165.76 mg, 640.75 μmol, 1.2 eq), the mixture was stirred at 25° C. for 1 hr. The mixture was concentrated to give a residue. The residue was purified by column chromatography (SiO₂, DCM/MeOH=100/1 to 1/50) and concentrated to give I-24-3 (0.37 g, 403.86 μmol, 75.64% yield, 93.76% purity) as a yellow solid. LCMS (Method D): Rt=0.495 min, [M+H]⁺=859.3. ¹H NMR (400 MHz, chloroform-d) δ 8.47 (d, J=8.0 Hz, 1H), 7.81-7.70 (m, 5H), 7.63-7.51 (m, 2H), 7.44-7.28 (m, 6H), 7.11-7.02 (m, 1H), 4.71-4.55 (m, 1H), 4.54-4.45 (m, 2H), 4.29 (s, 2H), 4.17-3.92 (m, 2H), 3.85-3.65 (m, 4H), 3.23-3.04 (m, 6H), 1.48 (s, 2H), 1.45 (s, 9H), 1.28 (d, J=7.2 Hz, 8H). ¹⁹F NMR (376 MHz, chloroform-d) δ −117.54.

### Step 3: Synthesis of I-24-4

A solution of I-24-3 (0.37 g, 430.74 μmol, 1 eq) in HCl/dioxane (4 M, 3 mL, 27.86 eq), was stirred at 25° C. for 0.5 hr. The mixture was concentrated to give I-24-4 (0.35 g, crude, HCl salt) as a yellow solid. LCMS (Method D): Rt=0.361 min, [M+H]⁺=759.3.

### Step 4: Synthesis of I-24-5

To a solution of 1-24-4 (0.35 g, 440.06 μmol, 1 eq, HCl salt) and 22-4 (135.89 mg, 528.08 μmol, 1.2 eq) in DMF (4 mL) was added EDCI (168.72 mg, 880.13 μmol, 2 eq), HOAt (29.95 mg, 220.03 μmol, 30.78 μL, 0.5 eq) and NMM (222.56 mg, 2.20 mmol, 241.91 μL, 5 eq) and the mixture was stirred at 25° C. for 1 hr. The residue was purified by column chromatography (SiO₂, DCM/MeOH=100/1 to 1/100) and the eluent was concentrated to give I-24-5 (0.2 g, 200.36 μmol, 45.53% yield, 99% purity) as a yellow oil. LCMS (Method D): Rt=0.550 min, [M+H]⁺=998.7. SFC (Method Z): Rt=1.711 min. ¹H NMR (400 MHz, methanol-d₄) δ 8.37 (d, J=7.6 Hz, 1H), 7.91-7.76 (m, 4H), 7.75-7.42 (m, 4H), 7.41-7.15 (m, 6H), 4.58 (d, J=4.8 Hz, 2H), 4.43-4.37 (m, 2H), 4.27-4.02 (m, 2H), 3.91-3.40 (m, 8H), 3.35 (s, 1H), 3.26-3.05 (m, 6H), 1.78-1.61 (m, 6H), 1.43 (s, 9H), 1.35-0.96 (m, 14H). ¹⁹F NMR (376 MHz, methanol-d₄) δ−120.68.

### Step 5: Synthesis of I-24-6

A solution of I-24-5 (0.2 g, 200.36 μmol, 1 eq) in HCl/dioxane (4 M, 2 mL, 39.93 eq) was stirred at 25° C. for 0.5 hr. The mixture was concentrated to give a residue. I-24-6 (0.18 g, 192.61 μmol, 96.13% yield, HCl) was obtained as a white solid. LCMS (Method D): Rt=0.432 min, [M+H]⁺=898.6.

### Step 6: Synthesis of I-24-7

To a solution of I-24-6 (0.180 g, 192.61 μmol, 1 eq, HCl salt) and Int-F (121.72 mg, 231.13 μmol, 1.2 eq) in DMF (2 mL) was added EDCI (73.85 mg, 385.22 μmol, 2 eq), HOAt (13.11 mg, 96.30 mol, 13.47 μL, 0.5 eq) and NMM (97.41 mg, 963.05 μmol, 105.88 μL, 5 eq) and the mixture was stirred at 25° C. for 1 hr. The mixture was poured into water (2 mL) and extracted with EA (2 mL*3), and the organic layers were dried over Na₂SO₄, filtered and concentrated to give I-24-7 (0.27 g, 191.94 μmol, 99.65% yield) as a yellow oil. LCMS (Method D): Rt=0.619 min, [M+H]⁺=1407.3.

### Step 7: Synthesis of I-24-8

To a solution of I-24-7 (0.25 g, 177.72 μmol, 1 eq) in DMF (2.5 mL) was added piperidine (15.13 mg, 177.72 μmol, 17.55 μL, 1 eq) and the mixture was stirred at 25° C. for 1 hr. The mixture was filtered to give a residue. The residue was purified by reversed-phase HPLC (0.1% FA condition) and lyophilized to give I-24-8 (0.2 g, 158.82 μmol, 89.36% yield, 94.05% purity) as a white solid. LCMS: Rt=0.472 min, [M+H]⁺=1184.7. SFC (Method AA): Rt=2.806 min, 3.317 min.

### Step 8: Synthesis of I-24

To a solution of I-24-8 (0.06 g, 50.66 μmol, 1 eq) in DCM (0.4 mL) was added HCl/dioxane (4 M, 200.00 μL, 15.79 eq) and the mixture was stirred at 25° C. for 0.5 hr. The mixture was concentrated to give a residue. The residue was purified by preparative HPLC (column: Phenomenex luna C18 150 mm*25 mm*10 um; mobile phase: [water(FA):ACN]; gradient: 20%-50% ACN over 10 min) and the eluent was lyophilized to give I-24 (21 mg, 19.37 μmol, 38.23% yield, 100% purity) as a white solid. LCMS (Method D): Rt=0.377 min, [M+H]⁺=1084.7; SFC (Method AB): Rt=1.676 min, 1.947 min. ¹H NMR (400 MHz, methanol-d₄) δ 8.50 (s, 2H), 8.37 (d, J=7.6 Hz, 1H), 7.98-7.92 (m, 1H), 7.89-7.81 (m, 2H), 7.77-7.70 (m, 2H), 7.53-7.43 (m, 3H), 7.39-7.33 (m, 1H), 7.28-7.14 (m, 2H), 6.62-6.53 (m, 2H), 4.58-4.47 (m, 1H), 4.38 (s, 2H), 4.34 (t, J=7.2 Hz, 1H), 4.17-4.10 (m, 4H), 4.02 (s, 1H), 3.98-3.93 (m, 2H), 3.93-3.82 (m, 1H), 3.80 (d, J=2.4 Hz, 4H), 3.77-3.68 (m, 3H), 3.53 (s, 2H), 3.36 (s, 2H), 3.27-3.15 (m, 3H), 2.97-2.91 (m, 2H), 2.87-2.67 (m, 2H), 2.08-2.02 (m, 1H), 1.87 (d, J=10.4 Hz, 4H), 1.78 (d, J=12.4 Hz, 2H), 1.69 (s, 5H), 1.55 (d, J=1.6 Hz, 2H), 1.49-1.42 (m, 3H), 1.39 (s, 6H), 1.35-1.23 (m, 3H), 1.23-1.01 (m, 3H). ¹⁹F NMR (400 MHz, methanol-d₄) δ −120.47

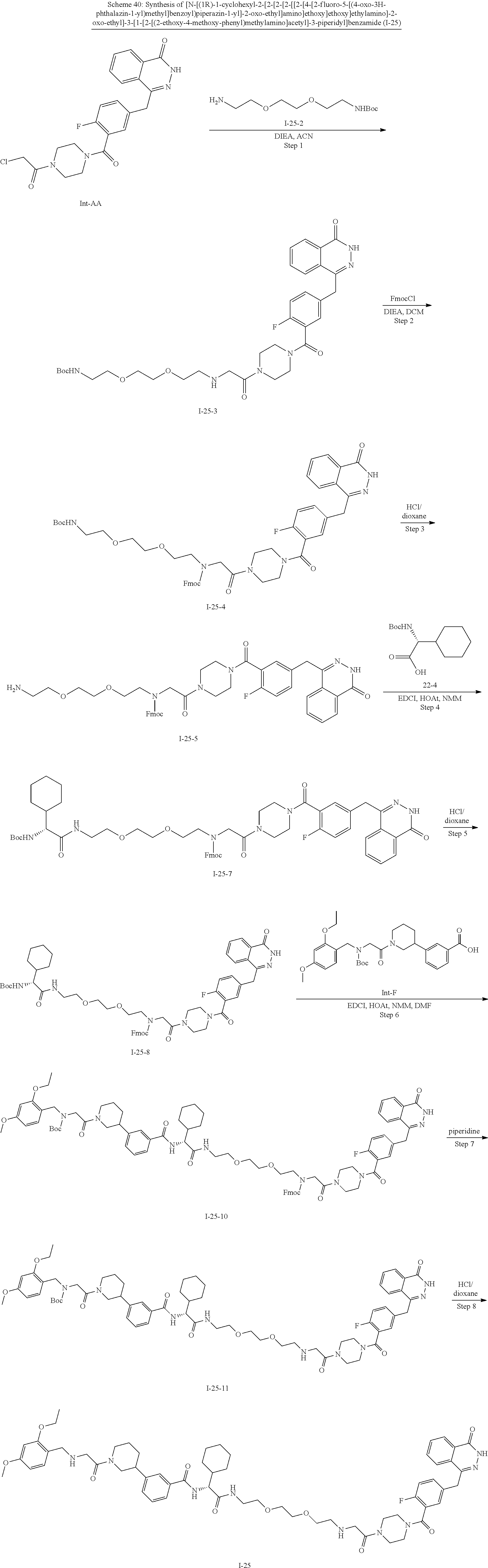

### Step 1: Synthesis of I-25-3

To a mixture of Int-AA (300 mg, 677.40 μmol, 1 eq) and I-25-2 (201.85 mg, 812.88 μmol, 1.2 eq) in ACN (3 mL) was added DIEA (350.20 mg, 2.71 mmol, 471.96 μL, 4 eq) at 25° C. and the mixture was stirred at 25° C. for 2 hr. The reaction mixture was concentrated to give a residue. The residue was purified by column chromatography (SiO₂, DCM:MeOH=1:0 to 4:1) and concentrated to give I-25-3 (360 mg, 549.85 μmol, 81.17% yield) as a white solid. LCMS (Method D): Rt=0.366 min, [M+H]⁺=655.4. ¹H NMR (400 MHz, chloroform-d) δ 10.75-9.99 (m, 1H), 8.46 (d, J=5.2 Hz, 1H), 7.83-7.70 (m, 3H), 7.33 (d, J=5.2 Hz, 2H), 7.06 (t, J=9.2 Hz, 1H), 5.30-5.18 (m, 1H), 4.29 (s, 2H), 3.83-3.26 (m, 20H), 3.16-3.05 (m, 2H), 1.44 (s, 9H).

### Step 2: Synthesis of I-25-4

To a mixture of 1-25-3 (360 mg, 549.85 μmol, 1 eq) in DCM (0.5 mL) was added DIEA (213.19 mg, 1.65 mmol, 287.32 μL, 3 eq) and Fmoc-Cl (170.69 mg, 659.82 μmol, 1.2 eq) and the mixture was stirred at 25° C. for 1 hr. The reaction mixture was concentrated to give a residue. The residue was purified by column chromatography (SiO₂, EA/MeOH=1/0 to 1/1) and concentrated to give I-25-4 (350 mg, 399.10 μmol, 72.58% yield) as a white solid. LCMS (Method D): Rt=0.544 min, [M+H]⁺=877.4. ¹H NMR (400 MHz, chloroform-d) δ 10.11-9.86 (m, 1H), 8.47 (d, J=8.4 Hz, 1H), 7.85-7.68 (m, 5H), 7.63-7.50 (m, 2H), 7.45-7.27 (m, 6H), 7.15-6.99 (m, 1H), 5.07-4.83 (m, 1H), 4.60-4.46 (m, 2H), 4.33-4.14 (m, 4H), 4.02-3.09 (m, 21H), 1.44 (s, 9H).

### Step 3: Synthesis of I-25-5

To a mixture of I-25-4 (350 mg, 399.10 μmol, 1 eq) in dioxane (3.5 mL) was added HCl/dioxane (4 M, 4 mL, 40.09 eq) at 25° C. and the mixture was stirred at 25° C. for 0.5 hr. The reaction mixture was concentrated to give I-25-5 (320 mg, crude, HCl) as a yellow gum. LCMS (Method D): Rt=0.391 min, [M+H]⁺=777.6.

### Step 4: Synthesis of I-25-7

To a mixture of I-25-5 (300 mg, 368.86 μmol, 1 eq, HCl) in DMF (3 mL) was added 22-4 (94.92 mg, 368.86 μmol, 1 eq), DIEA (238.36 mg, 1.84 mmol, 321.25 μL, 5 eq), EDCI (141.42 mg, 737.73 μmol, 2 eq), and HOAt (25.10 mg, 184.43 μmol, 25.80 μL, 0.5 eq) at 25° C. and the mixture was stirred at 25° C. for 1 hr. The reaction mixture was concentrated to give a residue. The residue was purified by column chromatography (SiO₂, EA/MeOH=1/0 to 2/1) and concentrated to give I-25-7 (120 mg, 118.09 μmol, 32.02% yield) as a white solid. LCMS (Method D): Rt=0.574 min, [M+H-Boc]⁺=916.4

LCMS (Method D): Rt=0.572 min, [M+H-Boc]⁺=916.5. SFC (Method S): Rt=1.137 min. ¹H NMR (400 MHz, chloroform-d) δ 10.85-10.30 (m, 1H), 8.45 (d, J=6.4 Hz, 1H), 7.83-7.66 (m, 5H), 7.63-7.45 (m, 2H), 7.42-7.27 (m, 6H), 7.10-6.98 (m, 1H), 4.60-4.44 (m, 2H), 4.33-4.14 (m, 4H), 3.99-3.85 (m, 2H), 3.80-3.65 (m, 3H), 3.62-3.49 (m, 9H), 3.47-3.35 (m, 6H), 3.13-3.04 (m, 1H), 2.80-2.69 (m, 1H), 1.88-1.84 (m, 1H), 1.41 (s, 9H), 1.32-0.90 (m, 10H).

### Step 5: Synthesis of I-25-8

To a mixture of I-25-7 (120 mg, 118.09 μmol, 1 eq) in dioxane (1 mL) was added HCl/dioxane (4 M, 1 mL, 33.87 eq) at 25° C. and the mixture was stirred at 25° C. for 0.5 hr. The reaction mixture was concentrated to give I-25-8 (110 mg, 115.48 μmol, 97.79% yield, HCl) as a yellow solid. LCMS (Method D): Rt=0.449 min, [M+H]⁺=916.4.

### Step 6: Synthesis of I-25-10

To a mixture of I-25-8 (110 mg, 115.48 μmol, 1 eq, HCl) in DMF (1 mL) was added Int-F (72.98 mg, 138.58 μmol, 1.2 eq), HOAt (31.44 mg, 230.97 μmol, 32.31 μL, 2 eq), EDCI (22.14 mg, 115.48 μmol, 1 eq) and NMM (58.40 mg, 577.42 μmol, 63.48 μL, 5 eq) at 25° C. and the mixture was stirred at 25° C. for 1 hr. The reaction mixture was diluted with H₂O (2 mL) and extracted with EtOAc (2 mL×3), and the combined organic layers were dried over Na₂SO₄, filtered and concentrated to give I-25-10 (150 mg, 105.29 μmol, 91.17% yield) as a yellow gum. LCMS (Method D): Rt=0.633 min, [M+H]⁺=1424.8.

### Step 7: Synthesis of I-25-11

To a mixture of I-25-10 (140 mg, 98.27 μmol, 1 eq) in DMF (1.4 mL) was added piperidine (16.73 mg, 196.54 μmol, 19.41 μL, 2 eq) at 25° C. and the mixture was stirred at 25° C. for 0.5 hr. The reaction mixture was concentrated to give a residue. The residue was purified by column chromatography (SiO₂, ethyl acetate/MeOH=1/0 to 1/1), and the eluent was concentrated to give I-25-11 (55 mg, 45.74 μmol, 46.55% yield) as a yellow solid. LCMS (Method D): Rt=0.491 min, [M+H]⁺=1202.7.

### Step 8: Synthesis of I-25

To a mixture of I-25-11 (45 mg, 37.42 μmol, 1 eq) in DCM (0.1 mL) was added HCl/dioxane (4 M, 25.71 μL, 2.75 eq) at 25° C. and the mixture was stirred at 25° C. for 0.5 hr. The reaction mixture was concentrated to give a residue. The residue was purified by reversed-phase HPLC (0.1% FA condition) and the eluent was concentrated and lyophilized to give I-25 (16.54 mg, 13.71 μmol, 36.63% yield, 91.371% purity) as a yellow solid. LCMS (Method D): Rt=0.399 min, [M+H]⁺=1102.5. SFC (Method S): Rt=0.910 min, 0.977 min. ¹H NMR (400 MHz, methanol-d₄) δ 8.40-8.32 (m, 1H), 7.97-7.91 (m, 1H), 7.90-7.79 (m, 2H), 7.78-7.69 (m, 2H), 7.53-7.39 (m, 3H), 7.38-7.32 (m, 1H), 7.30-7.22 (m, 1H), 7.16 (t, J=8.8 Hz, 1H), 6.65-6.50 (m, 2H), 4.58-4.43 (m, 1H), 4.37 (s, 3H), 4.20-4.07 (m, 4H), 4.04-3.91 (m, 3H), 3.89-3.83 (m, 1H), 3.80 (d, J=4.4 Hz, 4H), 3.74-3.68 (m, 4H), 3.64 (s, 4H), 3.61-3.50 (m, 5H), 3.40-3.34 (m, 3H), 3.23-3.05 (m, 4H), 2.94-2.69 (m, 2H), 2.09-1.98 (m, 1H), 1.90-1.66 (m, 9H), 1.50-1.39 (m, 3H), 1.35-1.05 (m, 6H). ¹⁹F NMR (376 MHz, methanol-d₄) δ−120.70.

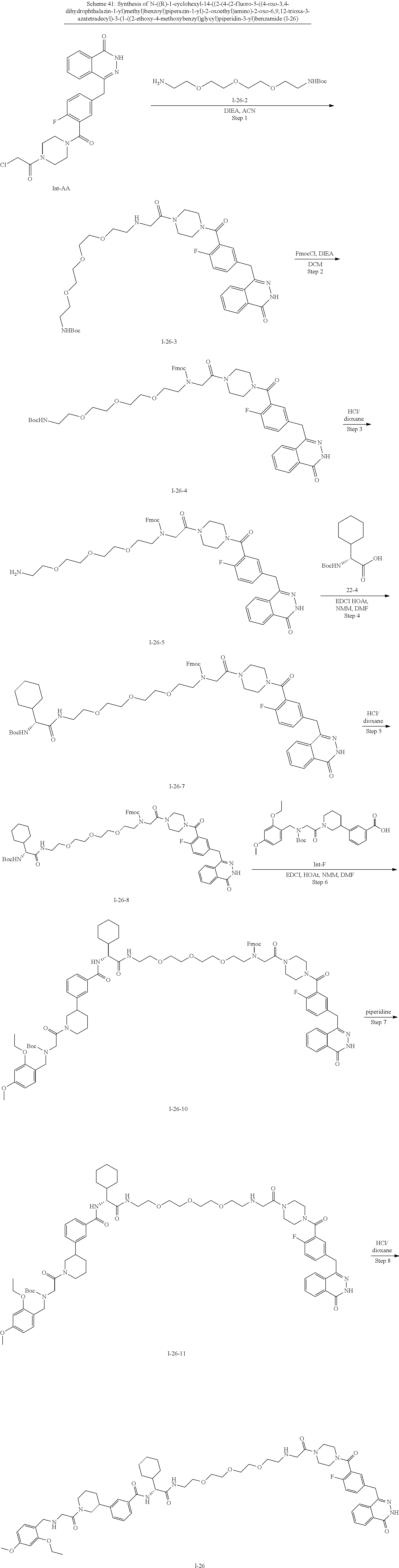

### Step 1: Synthesis of I-26-3

To a solution of Int-AA (300 mg, 677.40 μmol, 1 eq), I-26-2 (237.66 mg, 812.88 μmol, 1.2 ecanin ACN (3 mL) was added DIEA (350.20 mg, 2.71 mmol, 471.96 μL, 4 eq). The mixture was stirred at 40° C. for 2 hrs. The reaction mixture was added to H₂O (5 mL) and then extracted with DCM (10 mL*3), and the combined organic phase was dried over Na₂SO₄, filtered and concentrated to give a residue. The residue was purified by flash silica gel chromatography (20 g SepaFlash silica flash column; eluent: 0-100% ethyl acetate/petroleum ether gradient at 60 mL/min) and the eluent was concentrated to give I-26-3 (180 mg, 257.59 μmol, 38.03% yield) as a white solid. LCMS (Method F): Rt=0.547 min, [M+H]⁺=699.7.

### Step 2: Synthesis of I-26-4

To a solution of I-26-3 (180 mg, 257.59 μmol, 1 eq) in DCM (1.8 mL) was added DIEA (99.88 mg, 772.78 μmol, 134.60 μL, 3 eq) and Fmoc-Cl (79.97 mg, 309.11 μmol, 1.2 eq) at 0° C. The mixture was stirred at 25° C. for 1 hr. The reaction mixture was concentrated to give a residue. The residue was purified by flash silica gel chromatography (12 g SepaFlash silica flash column; eluent: 0-100% ethyl acetate/petroleum ether gradient at 60 mL/min) and the eluent was concentrated to give I-26-4 (200 mg, 201.95 μmol, 78.40% yield, 93% purity) as a yellow solid. LCMS (Method F): Rt=0.698 min, [M+H]⁺=921.4.

### Step 3: Synthesis of I-26-5

To a solution of I-26-4 (200 mg, 217.15 μmol, 1 eq) in dioxane (0.7 mL) was added HCl/dioxane (4 M, 1.4 mL, 25.79 eq). The mixture was stirred at 25° C. for 1 hr. The reaction mixture was concentrated under reduced pressure to give I-26-5 (180 mg, crude, HCl salt) as a white solid. LCMS (Method D): Rt=0.369 min, [M+H]⁺=821.7.

### Step 4: Synthesis of I-26-7

To a solution of I-26-5 (160 mg, 186.62 μmol, 1 eq, HCl), 22-4 (57.63 mg, 223.94 μmol, 1.2 eq) in DMF (1.6 mL) was added EDCI (71.55 mg, 373.24 μmol, 2 eq), HOAt (25.40 mg, 186.62 μmol, 26.11 μL, 1 eq) and NMM (94.38 mg, 933.09 μmol, 102.59 μL, 5 eq). The mixture was stirred at 25° C. for 1 hr. The mixture was purified by prep-HPLC (column: Phenomenex luna C18 150 mm*25 mm*10 um; mobile phase: [water(FA):ACN]; gradient: 54%-84% ACN over 10 min), and the eluent was concentrated to remove ACN and lyophilized to give I-26-7 (200 mg, 181.10 μmol, 97.04% yield, 96% purity) as a yellow solid. LCMS (Method D): Rt=0.502 min, [M+H-Boc]⁺=960.7 LCMS (Method D): Rt=0.500 min, [M+H-Boc]⁺=960.9. SFC (Method S): Rt=1.195 min. ¹H NMR (400 MHz, DMSO-d₆) δ 12.60 (d, J=4.4 Hz, 1H), 8.26 (d, J=8.0 Hz, 1H), 7.92-7.80 (m, 5H), 7.66 (d, J=7.2 Hz, 1H), 7.57 (d, J=7.2 Hz, 1H), 7.50-7.18 (m, 7H), 6.56 (d, J=8.8 Hz, 1H), 4.43-4.30 (m, 4H), 4.21-3.94 (m, 4H), 3.75 (t, J=7.6 Hz, 1H), 3.67-3.52 (m, 3H), 3.46 (s, 10H), 3.27-3.10 (m, 11H), 1.66-1.50 (m, 6H), 1.35 (s, 9H), 1.06 (d, J=7.2 Hz, 4H). ¹⁹F NMR (376 MHz, DMSO-d₆) δ−119.772.

### Step 5: Synthesis of I-26-8

To a solution of I-26-7 (118 mg, 111.30 μmol, 1 eq) in DCM (0.5 mL) was added HCl/dioxane (4 M, 1 mL, 35.94 eq). The mixture was stirred at 25° C. for 1 hr. The reaction mixture was concentrated under reduced pressure to give I-26-8 (120 mg, crude, HCl salt) as a yellow solid. LCMS (Method D): Rt=0.412 min, [M+H-Boc]⁺=1060.6.

### Step 6: Synthesis of I-26-10

To a solution of I-26-8 (120 mg, 120.41 μmol, 1 eq, HCl), Int-F (76.10 mg, 144.50 μmol, 1.2 eq) in DMF (1.2 mL) was added EDCI (46.17 mg, 240.83 μmol, 2 eq), HOAt (16.39 mg, 120.41 μmol, 16.84 L, 1 eq) and NMM (60.90 mg, 602.07 μmol, 66.19 μL, 5 eq). The mixture was stirred at 25° C. for 1 hr. The reaction mixture was filtered to give a residue. The residue was purified by flash silica gel chromatography (20 g SepaFlash silica flash column; eluent: 0-100% methanol/dichloromethane ether gradient at 40 mL/min) and the eluent was concentrated to give I-26-10 (180 mg, 102.95 μmol, 85.49% yield, 84% purity) as a yellow solid. LCMS (Method D): Rt=0.548 min, [M+H]⁺=1469.8.

### Step 7: Synthesis of I-26-11

To a solution of I-26-10 (180 mg, 102.95 μmol, 1 eq) in DMF (1.8 mL) was added piperidine (8.77 mg, 102.95 μmol, 10.17 μL, 1 eq). The mixture was stirred at 25° C. for 1 hr. The reaction mixture was added to H₂O (3 mL) and then extracted with DCM (3 mL*3), and the combined organic phase was dried over Na₂SO₄, filtered and concentrated to give a residue. The residue was purified by prep-HPLC (column: Welch Ultimate C18 150 mm*25 mm*5 um; mobile phase: [water (TFA):can]; gradient: 40%-70% ACN over 10 min) and the eluent was concentrated to remocanACN and lyophilized to give I-26-11 (80 mg, 62.26 μmol, 60.47% yield, 97% purity) as a yellow solid. LCMS (Method D): Rt=0.444 min, [M+H]⁺=1247.2. SFC (Method U): Rt=4.366 min, 4.942 min.

### Step 8: Synthesis of I-26

To a solution of I-26-11 (70 mg, 56.16 μmol, 1 eq) in DCM (0.7 mL) was added HCl/dioxane (4 M, 0.35 mL, 24.93 eq). The mixture was stirred at 25° C. for 1 hr. The reaction mixture was concentrated under reduced pressure to give a residue. The crude product was purified by reversed-phase HPLC (0.1% FA condition) and the eluent was concentrated to remove ACN and lyophilized to give I-26 (15 mg, 11.78 μmol, 20.98% yield, 99% purity, TFA salt) as a white solid. LCMS (Method D): Rt=0.398 min, [M+H]⁺=1146.7. SFC (Method V): Rt=3.810 min, 7.234 min. ¹H NMR (400 MHz, methanol-d₄) δ 8.51 (s, 1H), 8.35 (d, J=7.6 Hz, 1H), 7.97-7.91 (m, 1H), 7.90-7.79 (m, 2H), 7.78-7.69 (m, 2H), 7.53-7.39 (m, 3H), 7.38-7.32 (m, 1H), 7.28-7.23 (m, 1H), 7.16 (t, J=8.8 Hz, 1H), 6.67-6.46 (m, 2H), 4.57-4.44 (m, 1H), 4.42-4.33 (m, 3H), 4.21-4.06 (m, 4H), 4.03-3.86 (m, 4H), 3.79 (d, J=5.2 Hz, 4H), 3.72 (d, J=4.0 Hz, 4H), 3.69-3.57 (m, 10H), 3.54 (d, J=12.0 Hz, 4H), 3.48-3.41 (m, 1H), 3.35 (d, J=6.8 Hz, 3H), 3.23-3.09 (m, 3H), 2.95-2.68 (m, 2H), 2.02 (d, J=11.2 Hz, 1H), 1.91-1.60 (m, 9H), 1.48-1.40 (m, 3H), 1.32-1.06 (m, 5H). ¹⁹F NMR (376 MHz, methanol-d₄) δ −120.65.

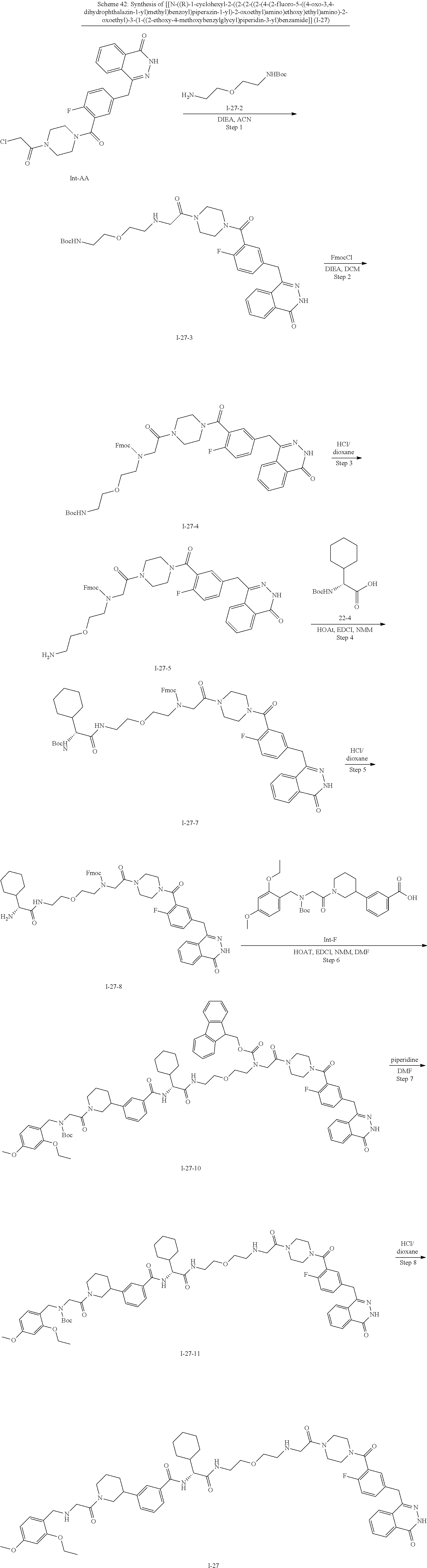

### Step 1: Synthesis of I-27-3

To a solution of Int-AA (200 mg, 451.60 μmol, 1 eq) and I-27-2 (138.37 mg, 677.40 μmol, 1.5 eq) in ACN (2 mL) was added DIEA (175.10 mg, 1.35 mmol, 235.98 μL, 3 eq). The mixture was stirred at 25° C. for 4 hr. The reaction mixture was quenched by addition of H₂O (2 mL), and then extracted with EA 8 mL (4 mL*2). The combined organic layers were washed with the saturated solution of NaCl (2 mL*2), dried over Na₂SO₄, filtered and concentrated under reduced pressure to give a residue. The residue was purified by flash silica gel chromatography (20 g SepaFlash silica flash column; eluent: 0-10% MeOH/DCM gradient at 20 mL/min). The eluent was concentrated under reduced pressure to give the product. I-27-3 (300 mg, 397.92 μmol, 88.11% yield, 81% purity) was obtained as a yellow oil. LCMS (Method D): Rt=0.294 min, [M+H]⁺=611.3. ¹H NMR (400 MHz, chloroform-d) δ 8.54-8.40 (m, 1H), 7.89-7.71 (m, 3H), 7.37-7.29 (m, 2H), 7.11-7.02 (m, 1H), 4.29 (s, 2H), 3.79-3.75 (m, 2H), 3.74-3.64 (m, 4H), 3.62-3.56 (m, 2H), 3.56-3.53 (m, 3H), 3.43-3.39 (m, 1H), 3.37-3.28 (m, 4H), 3.23-3.20 (m, 1H), 3.12-2.92 (m, 2H), 1.43 (s, 9H).

### Step 2: Synthesis of I-27-4

To a solution of I-27-3 (300 mg, 491.26 μmol, 1 eq) in DCM (3 mL) was added DIEA (190.48 mg, 1.47 mmol, 256.71 μL, 3 eq) and Fmoc-Cl (190.63 mg, 736.89 μmol, 1.5 eq). The mixture was stirred at 25° C. for 2 hr. The reaction mixture was concentrated under reduced pressure to remove DCM. The residue was diluted with H₂O (5 mL) and extracted with EA (10 mL*2) dried over Na₂SO₄, filtered and concentrated under reduced pressure to give a residue. The residue was purified by flash silica gel chromatography (20 g SepaFlash silica flash column; eluent: 0-20% EtOH/DCM at 100 mL/min) and the eluent was concentrated under reduced pressure to give the product. I-27-4 (400 mg, 417.81 μmol, 85.05% yield, 87% purity) was obtained as an orange oil. LCMS (Method D): Rt=0.463 min, [M+H]⁺*=833.7. ¹H NMR (400 MHz, chloroform-d) δ 8.50-8.44 (m, 1H), 7.80-7.75 (m, 4H), 7.64-7.52 (m, 3H), 7.45-7.29 (m, 8H), 7.13-7.01 (m, 1H), 5.00-4.79 (m, 1H), 4.62-4.50 (m, 2H), 4.42 (d, J=6.4 Hz, 1H), 4.28 (s, 2H), 4.26-4.16 (m, 2H), 3.61-3.46 (m, 6H), 3.45-3.27 (m, 9H), 1.43 (s, 9H).

### Step 3: Synthesis of I-27-5

To a solution of I-27-4 (400 mg, 480.24 μmol, 1 eq) in DCM (4 mL) was added HCl/dioxane (4 M, 4 mL). The mixture was stirred at 25° C. for 1 hr. The mixture was filtered and concentrated to give a residue. The crude product was used in the next step without further purification. I-27-5 (350 mg, crude, HCl) was obtained as a colorless oil. LCMS (Method D): Rt=0.386 min, [M+H]⁺=733.6.

### Step 4: Synthesis of I-27-7

To a solution of 22-4 (91.99 mg, 357.49 μmol, 1.1 eq) in DMF (1 mL) was added HOAt (88.47 mg, 649.98 μmol, 90.92 μL, 2 eq), EDCI (311.50 mg, 1.62 mmol, 5 eq), NMM (361.60 mg, 3.57 mmol, 393.04 L, 11 eq) and I-27-5 (250 mg, 324.99 μmol, 1 eq, HCl). The mixture was stirred at 25° C. for 1 hr. The residue was diluted with H₂O (10 mL) and extracted with EA (10 mL*3). The combined organic layers were washed with NaCl (25 mL*2), dried over Na₂SO₄, filtered and concentrated under reduced pressure to give a residue. The residue was purified by prep-HPLC (FA condition), the solvent (ACN) removed by rotary evaporation, and then extracted with EA (3×10 mL). The organic layer was dried over Na₂SO₄, filtered and concentrated under vacuum to give the product. I-27-7 (180 mg, 184.42 μmol, 56.75% yield, 99.6% purity) was obtained as a colorless oil. LCMS (Method D): Rt=0.534 min, [M+H]⁺=972.5. SFC (Method S): Rt=0.834 min, 1.955 min. ¹HNMR (400 MHz, chloroform-d) δ 10.82-10.34 (m, 1H), 8.50-8.43 (m, 1H), 7.84-7.68 (m, 5H), 7.61-7.51 (m, 2H), 7.44-7.28 (m, 6H), 7.15-7.00 (m, 1H), 6.85-6.55 (m, 1H), 5.37-5.18 (m, 1H), 4.64-4.49 (m, 2H), 4.37-4.16 (m, 4H), 4.01-3.51 (m, 8H), 3.50-3.40 (m, 3H), 3.36-3.06 (m, 6H), 1.70 (s, 4H), 1.63 (d, J=11.2 Hz, 2H), 1.44 (s, 1H), 1.40 (d, J=10.0 Hz, 9H), 1.15-1.05 (m, 2H), 1.00-0.93 (m, 1H), 0.92-0.81 (m, 1H). ¹⁹F NMR (376 MHz, chloroform-d) 6-117.505.

### Step 5: Synthesis of I-27-8

To a solution of I-27-7 (110 mg, 113.16 μmol, 1 eq) in DCM (1 mL) was added HCl/dioxane (4 M, 1 mL). The mixture was stirred at 25° C. for 0.5 hr. The reaction mixture was concentrated under reduced pressure to give I-27-8 (90 mg, 99.07 μmol, 87.55% yield, HCl) as a light yellow solid. LCMS (Method D): Rt=0.405 min, [M+H]⁺=872.8.

### Step 6: Synthesis of I-27-10

To a solution of Int-F (62.61 mg, 118.88 μmol, 1.2 eq) in DMF (1 mL) was added HOAt (26.97 mg, 198.14 μmol, 27.72 μL, 2 eq), EDCI (94.96 mg, 495.35 μmol, 5 eq), NMM (100.21 mg, 990.70 μmol, 108.92 μL, 10 eq) and I-27-8 (90 mg, 99.07 μmol, 1 eq, HCl). The mixture was stirred at 25° C. for 12 hr. The reaction was diluted with H₂O 5 mL and extracted with EA 15 mL (5 mL*3). The combined organic layers were washed with brine 5 mL, dried over Na₂SO₄, filtered and concentrated under reduced pressure to give a residue. The residue was purified by prep-HPLC (FA condition) to give I-27-10 (100 mg, 59.60 μmol, 60.16% yield, 82.279% purity) as a white solid. LCMS (Method D): Rt=0.588 min, [M+H]⁺⁺=1380.9.

### Step 7: Synthesis of I-27-11

To a solution of I-27-10 (100.00 mg, 72.43 μmol, 1 eq) in THF (1 mL) was added piperidine (86.22 mg, 0.1 mL). The mixture was stirred at 25° C. for 0.5 hr. The reaction mixture was concentrated under reduced pressure to remove THF. The residue was diluted with H₂O (3 mL) and extracted with EA (5 mL*2). The combined organic layers were dried over Na₂SO₄, filtered and concentrated under reduced pressure to give a residue. The residue was purified by flash silica gel chromatography (12 g SepaFlash silica flash column; eluent: 0-100% ethyl acetate/petroleum ether gradient at 50 mL/min) and the eluent was concentrated under reduced pressure to give the product. I-27-11 (30 mg, 24.85 μmol, 34.31% yield, 95.943% purity) was obtained as a colorless oil. LCMS (Method D): Rt=0.468 min, [M+H]⁺=1158.7

### Step 8: Synthesis of I-27

To a solution of I-27-11 (30 mg, 25.90 μmol, 1 eq) in DCM (0.3 mL) was added HCl/dioxane (4 M, 0.15 mL). The mixture was stirred at 25° C. for 1 hr. The reaction mixture was concentrated under reduced pressure to remove DCM. The residue was diluted with NH₃·H₂O (0.5 mL) to adjust the pH to 8 and concentrated under reduced pressure to give a residue. The residue was purified by prep-HPLC (FA condition) and the eluent was concentrated under reduced pressure to remove ACN and the lyophilized to give the product. I-27 (18.80 mg, 16.37 μmol, 63.20% yield, 96.142% purity, FA) was obtained as a white solid. LCMS (Method D): Rt=0.368 min, [M+H]⁺=1058.6. SFC (Method T): Rt=3.00 min, 5.121 min. ¹H NMR (400 MHz, methanol-d₄) δ 8.-9-8.47 (m, 1H), 8.36 (d, J=7.6 Hz, 1H), 7.-7-7.91 (m, 1H), 7.-0-7.79 (m, 2H), 7.-7-7.67 (m, 2H), 7.-4-7.40 (m, 3H), 7.36 (s, 1H), 7.-6-7.11 (m, 2H), 6.-1-6.47 (m, 2H), 4.-7-4.45 (m, 1H), 4.37 (s, 3H), 4.-5-3.98 (m, 4H), 3.-2-3.82 (m, 2H), 3.-1-3.65 (m, 9H), 3.-5-3.49 (m, 7H), 3.-5-3.35 (m, 3H), 3.-5-3.09 (m, 2H), 2.-3-2.85 (m, 2H), 2.-4-2.63 (m, 2H), 2.-9-1.97 (m, 1H), 1.-3-1.82 (m, 4H), 1.-1-1.75 (m, 2H), 1.-2-1.57 (m, 3H), 1.-8-1.39 (m, 3H), 1.-4-1.20 (m, 3H), 1.-9-1.07 (m, 2H). ¹⁹F NMR (376 MHz, DMSO-d₆) δ −120.605.

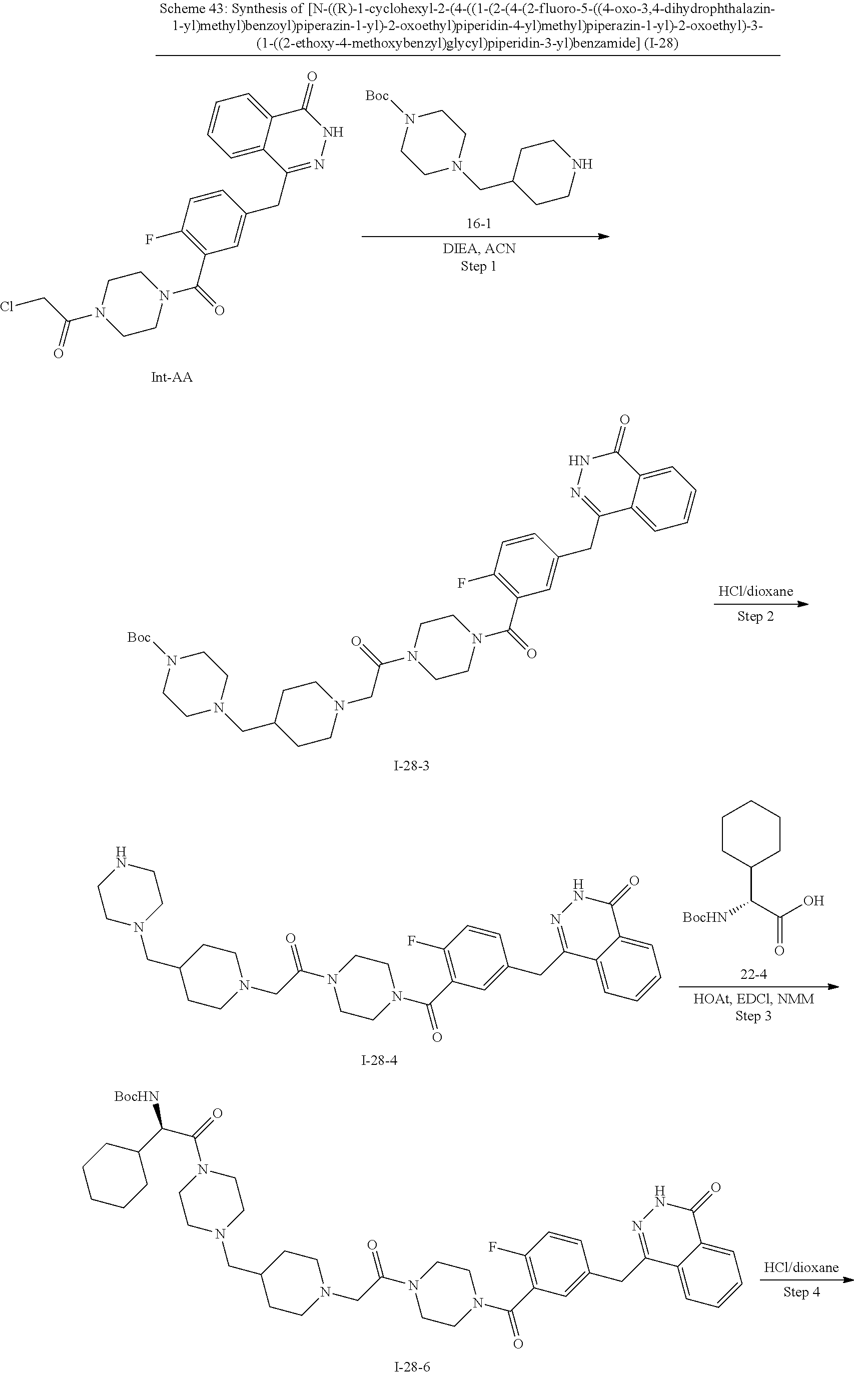 [SMILES: O=C(CCl)N1CCN(C(=O)c2cc(Cc3n[nH]c(=O)c4ccccc34)ccc2F)CC1 ; CC(C)(C)OC(=O)N1CCN(CC2CCNCC2)CC1 ; CC(C)(C)OC(=O)N1CCN(CC2CCN(CC(=O)N3CCN(C(=O)c4cc(Cc5n[nH]c(=O)c6ccccc56)ccc4F)CC3)CC2)CC1 ; O=C(CN1CCC(CN2CCNCC2)CC1)N1CCN(C(=O)c2cc(Cc3n[nH]c(=O)c4ccccc34)ccc2F)CC1 ; CC(C)(C)OC(=O)N[C@@H](C(=O)O)C1CCCCC1 ; CC(C)(C)OC(=O)N[C@@H](C(=O)N1CCN(CC2CCN(CC(=O)N3CCN(C(=O)c4cc(Cc5n[nH]c(=O)c6ccccc56)ccc4F)CC3)CC2)CC1)C1CCCCC1]

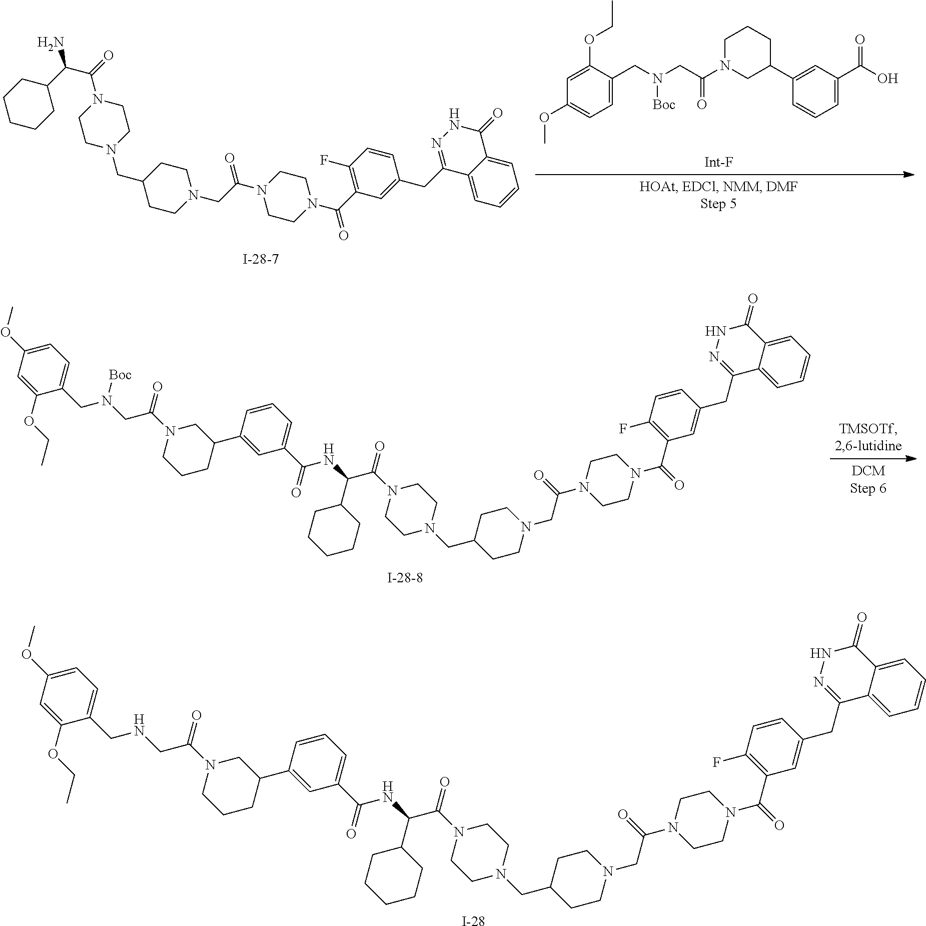

### Step 1: Synthesis of I-28-3

To a solution of 16-1 in ACN (3 mL) was added Int-AA (200 mg, 451.60 μmol, 1 eq) and DIEA (175.10 mg, 1.35 mmol, 235.98 μL, 3 eq). The mixture was stirred at 25° C. for 6 hr. The reaction mixture was concentrated under reduced pressure to remove ACN. The residue was diluted with H₂O (10 mL) and extracted with EA (5 mL*3). The combined organic layers were washed with brine (5 mL*3), dried over Na₂SO₄, filtered and concentrated under reduced pressure to give a residue. The residue was purified by prep-HPLC (FA condition) and the eluent was concentrated under reduced pressure to give I-28-3 (320 mg, 432.07 μmol, 95.68% yield, 99.3% purity, FA salt) as a yellow oil. LCMS (Method D): Rt=0.277 min, [M+H]⁺=690.5. ¹H NMR (400 MHz, chloroform-d) δ 11.45-10.71 (m, 1H), 8.48-8.43 (m, 1H), 7.84-7.69 (m, 3H), 7.37-7.29 (m, 2H), 7.10-7.00 (m, 1H), 4.30 (s, 2H), 4.02-3.36 (m, 14H), 3.35-3.12 (m, 4H), 2.67-2.49 (m, 6H), 2.45-2.36 (m, 2H), 1.87 (d, J=12.6 Hz, 2H), 1.73 (s, 1H), 1.46 (s, 9H). ¹⁹F NMR (376 MHz, chloroform-d) δ −117.79.

### Step 2: Synthesis of I-28-4

To a solution of I-28-3 (310 mg, 421.29 μmol, 1 eq, FA salt) in DCM (3 mL) was added HCl/dioxane (4 M, 3 mL). The mixture was stirred at 25° C. for 1 hr. The reaction mixture was concentrated under reduced pressure to remove DCM. The crude product was used into the next step without further purification. I-28-4 (300 mg, crude, HCl salt) was obtained as a white solid. LCMS (Method F): Rt=0.430 min, [M+H]⁺=590.4. ¹H NMR (400 MHz, DMSO-d₆) δ 8.30-8.21 (m, 1H), 8.01-7.75 (m, 3H), 7.47 (s, 1H), 7.41-7.31 (m, 1H), 7.29-7.21 (m, 1H), 4.34 (s, 2H), 3.50 (d, J=1.2 Hz, 8H), 3.32-3.15 (m, 8H), 3.14-2.92 (m, 6H), 2.14 (d, J=12.8 Hz, 3H), 1.67-1.48 (m, 5H). ¹⁹F NMR (376 MHz, DMSO-d₆) δ −119.69.

### Step 3: Synthesis of I-28-6

To a solution of 22-4 (82.19 mg, 319.41 μmol, 1 eq) in DMF (1 mL) was added HOAt (86.95 mg, 638.81 μmol, 89.36 μL, 2 eq) and EDCI (306.15 mg, 1.60 mmol, 5 eq). Then I-28-4 (200 mg, 319.41 μmol, 1 eq, HCl salt) and NMM (323.07 mg, 3.19 mmol, 351.16 μL, 10 eq) in DMF (1 mL) was added. The mixture was stirred at 25° C. for 1 hr. The reaction mixture was diluted with H₂O (5 mL) and extracted with EA (5 mL*3). The combined organic layers were washed with brine (5 mL*3), dried over Na₂SO₄, filtered and concentrated under reduced pressure to give a residue. The residue was purified by prep-HPLC (FA condition) and the eluent was concentrated under reduced pressure to give I-28-6 (190 mg, 213.20 μmol, 66.75% yield, 98.19% purity, FA salt) as a white solid. LCMS (Method D): Rt=0.299 min, [M+H]⁺=829.4. SFC (Method P): Rt=0.608 min. ¹H NMR (400 MHz, chloroform-d) δ 10.47-9.96 (m, 1H), 8.51-8.42 (m, 1H), 8.15 (s, 1H), 7.85-7.67 (m, 3H), 7.41-7.29 (m, 2H), 7.12-7.00 (m, 1H), 5.31 (d, J=8.0 Hz, 1H), 4.51-4.41 (m, 1H), 4.29 (s, 2H), 3.84-3.75 (m, 2H), 3.73-3.67 (m, 2H), 3.66-3.49 (m, 6H), 3.40-3.24 (m, 4H), 3.02-2.94 (m, 2H), 2.45 (d, J=16.8 Hz, 5H), 2.27-2.23 (m, 2H), 1.87-1.71 (m, 5H), 1.65-1.51 (m, 4H), 1.44 (s, 9H), 1.34-1.01 (m, 8H). ¹⁹F NMR (376 MHz, chloroform-d) δ −117.59.

### Step 4: Synthesis of I-28-7

To a solution of I-28-6 (180 mg, 217.13 μmol, 1 eq) in DCM (2 mL) was added HCl/dioxane (4 M, 2 mL). The mixture was stirred at 25° C. for 0.5 hr. The reaction mixture was concentrated under reduced pressure to remove DCM. The crude product was used in the next step without further purification. I-28-7 (160 mg, 209.05 μmol, 96.28% yield, HCl salt) was obtained as a white solid. LCMS (Method D): Rt=0.229 min, [M+H]⁺=729.4.

### Step 5: Synthesis of I-28-9

To a solution of I-28-7 (100 mg, 130.66 μmol, 1 eq, HCl salt) and Int-F (68.81 mg, 130.66 μmol, 1 eq) in DMF (1 mL) was added HOAt (35.57 mg, 261.32 μmol, 36.55 μL, 2 eq), EDCI (125.24 mg, 653.29 mol, 5 eq), and NMM (132.16 mg, 1.31 mmol, 143.65 μL, 10 eq). The mixture was stirred at 25° C. for 1 hr. The reaction mixture was diluted with H₂O (5 mL) and extracted with EA (5 mL*3). The combined organic layers were washed with brine (5 mL*3), dried over Na₂SO₄, filtered and concentrated under reduced pressure to give a residue. The residue was purified by prep-HPLC (FA condition) and the eluent was concentrated under reduced pressure to give I-28-9 (90 mg, 72.73 μmol, 55.66% yield, 99% purity) as a yellow solid. LCMS (Method D): Rt=0.428 min, [M+H]⁺=1237.9. SFC: Rt=3.079, 3.219 min.

### Step 6: Synthesis of I-28

To a solution of I-28-9 (60 mg, 48.48 μmol, 1 eq) in DCM (0.6 mL) was added 2,6-dimethylpyridine (1.56 mg, 14.55 μmol, 1.69 μL, 0.3 eq) and trimethylsilyl trifluoromethanesulfonate (16.16 mg, 72.73 μmol, 13.14 μL, 1.5 eq). The mixture was stirred at 0° C. for 0.5 hr. The reaction mixture was concentrated under reduced pressure to remove DCM. The residue was diluted with H₂O (2 mL) and extracted with EA (5 mL*3). The combined organic layers were dried over Na₂SO₄, filtered and concentrated under reduced pressure to give a residue. The residue was purified by prep-HPLC (column: Waters Xbridge C18 150 mm*50 mm*10 um; mobile phase: [water(NH₃H₂O):ACN]; gradient: 45%-75% ACN over 11 min) and the eluent was concentrated under reduced pressure to give I-28 (24.32 mg, 21.38 mol, 44.10% yield, 99% purity) as a white solid. LCMS (Method D): Rt=0.341 mm, [M+H]⁺=1137.8. SFC (Method R): Rt=6.415, 7.756 min. ¹H NMR (400 MHz, methanol-d₄) δ 8.41-8.33 (m, 1H), 7.99-7.91 (m, 1H), 7.89-7.80 (m, 2H), 7.78-7.65 (m, 2H), 7.51-7.41 (m, 3H), 7.38 (s, 1H), 7.20-7.09 (m, 2H), 6.56-6.42 (m, 2H), 4.93-4.89 (m, 1H), 4.58-4.46 (m, 1H), 4.38 (s, 2H), 4.11-4.00 (m, 2H), 3.80 (d, J=5.2 Hz, 3H), 3.77-3.75 (m, 4H), 3.73 (d, J=5.2 Hz, 4H), 3.66 (d, J=1.6 Hz, 1H), 3.59-3.35 (m, 6H), 3.26-3.17 (m, 2H), 3.15-3.03 (m, 1H), 2.95-2.83 (m, 2H), 2.80-2.60 (m, 2H), 2.52-2.33 (m, 4H), 2.26-2.15 (m, 2H), 2.12-1.99 (m, 3H), 1.95-1.82 (m, 4H), 1.81-1.65 (m, 7H), 1.63-1.50 (m, 2H), 1.47-1.38 (m, 3H), 1.35-1.16 (m, 6H), 1.10 (d, J=12.0 Hz, 2H). ¹⁹F NMR (376 MHz, methanol-d₄) δ −120.77.

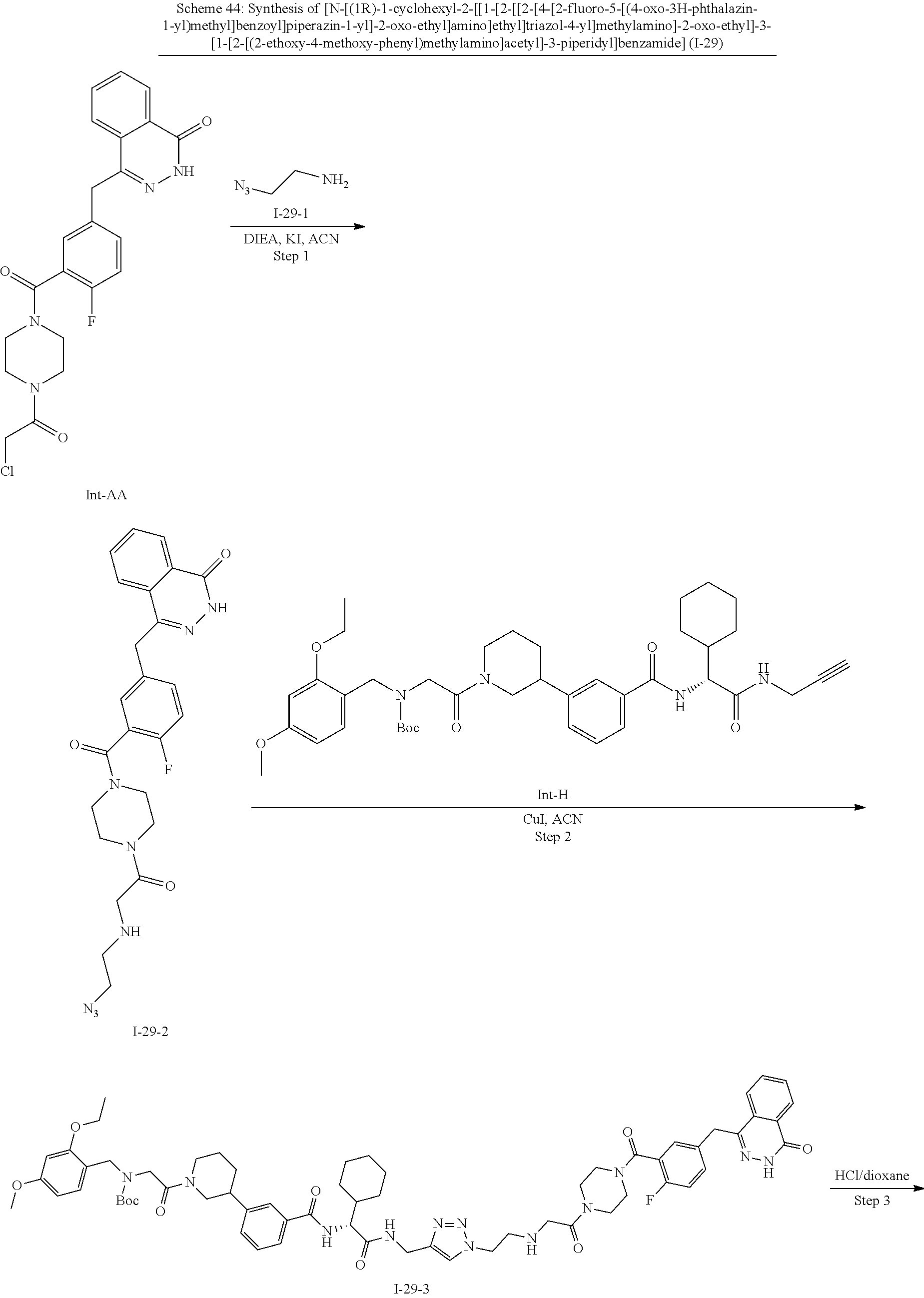

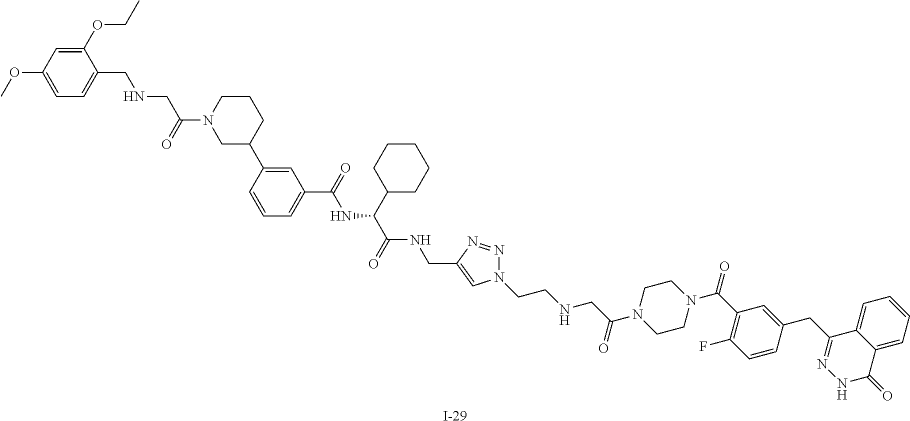

### Step 1: Synthesis of I-29-2

To a solution of Int-AA (166.04 mg, 1.35 mmol, 2 eq, HCl) and I-29-1 (300.00 mg, 677.40 μmol, 1 eq) in ACN (4 mL) was added DIEA (437.75 mg, 3.39 mmol, 589.95 μL, 5 eq) and KI (112.45 mg, 677.40 μmol, 1 eq). The mixture was stirred at 40° C. for 2 hr. The crude product was purified by reverse phase HPLC (0.1% FA condition) and the eluent was lyophilized to give I-29-2 (150 mg, 304.57 μmol, 44.96% yield) as a yellow solid. LCMS (Method D): Rt=0.264 min, [M+H]⁺=493.3. ¹H NMR (400 MHz, chloroform-d) δ 10.82-10.56 (m, 1H), 8.52-8.42 (m, 1H), 7.84-7.77 (m, 2H), 7.76-7.71 (m, 1H), 7.40-7.29 (m, 2H), 7.08-7.04 (m, 1H), 4.30 (s, 2H), 3.90-3.71 (m, 3H), 3.62 (s, 2H), 3.56-3.49 (m, 4H), 3.42-3.31 (m, 4H), 2.98-2.85 (m, 2H).

### Step 2: Synthesis of I-29-3

A mixture of Int-H (95 mg, 135.16 μmol, 1 eq), I-29-2 (66.57 mg, 135.16 μmol, 1 eq), and CuI (5.15 mg, 27.03 μmol, 0.2 eq) in ACN (4 mL) was stirred at 60° C. for 1 hr under N₂ atmosphere and in darkness. DCM (4 mL) and water (3 mL) were added and the layers were separated. The aqueous phase was extracted with DCM (4 mL×2), dried over Na₂SO₄, filtered, and concentrated under vacuum. The crude product was purified by reverse phase HPLC (0.1% FA) to give I-29-3 (85 mg, 67.98 μmol, 50.30% yield, 95.608% purity) as a yellow solid. LCMS (Method D): Rt=0.4587 min, [M+H]⁺=1196.6.

### Step 3: Synthesis of I-29

To a solution of I-29-3 (75 mg, 62.74 μmol, 1 eq) in DCM (3 mL) was added HCl/dioxane (4 M, 750.00 μL, 47.82 eq). The mixture was stirred at 25° C. for 0.2 hr. The mixture was concentrated under vacuum. The crude product was purified by reverse phase HPLC (0.10% FA) and the eluent was lyophilized to give I-29 (11.5 mg, 9.64 μmol, 15.36% yield, 95.644% purity, FA) as a white solid. LCMS (Method D): Rt=0.361 min, [M+H]⁺=1095.6. SFC (Method AC): Rt=3.954 min, 7.658 min. ¹H NMR (400 MHz, methanol-d₄) δ 8.63-8.41 (m, 1H), 8.36 (d, J=8.0 Hz, 1H), 7.97-7.90 (m, 2H), 7.89-7.80 (m, 2H), 7.76-7.70 (m, 2H), 7.51-7.41 (m, 3H), 7.37 (s, 1H), 7.28-7.23 (m, 1H), 7.16 (s, 1H), 6.61 (s, 2H), 4.57-4.43 (m, 5H), 4.37 (s, 2H), 4.17-4.06 (m, 4H), 4.02-3.89 (m, 2H), 3.79 (d, J=5.2 Hz, 3H), 3.70 (s, 3H), 3.61 (s, 1H), 3.48 (s, 3H), 3.40 (s, 1H), 3.28-3.13 (m, 4H), 3.08 (d, J=4.8 Hz, 2H), 2.91-2.68 (m, 2H), 2.07-2.00 (m, 1H), 1.85 (s, 4H), 1.79-1.73 (m, 2H), 1.67 (d, J=11.2 Hz, 3H), 1.48-1.40 (m, 3H), 1.36-1.01 (m, 6H). ¹⁹F NMR (376 MHz, methanol-d₄) δ −120.74.

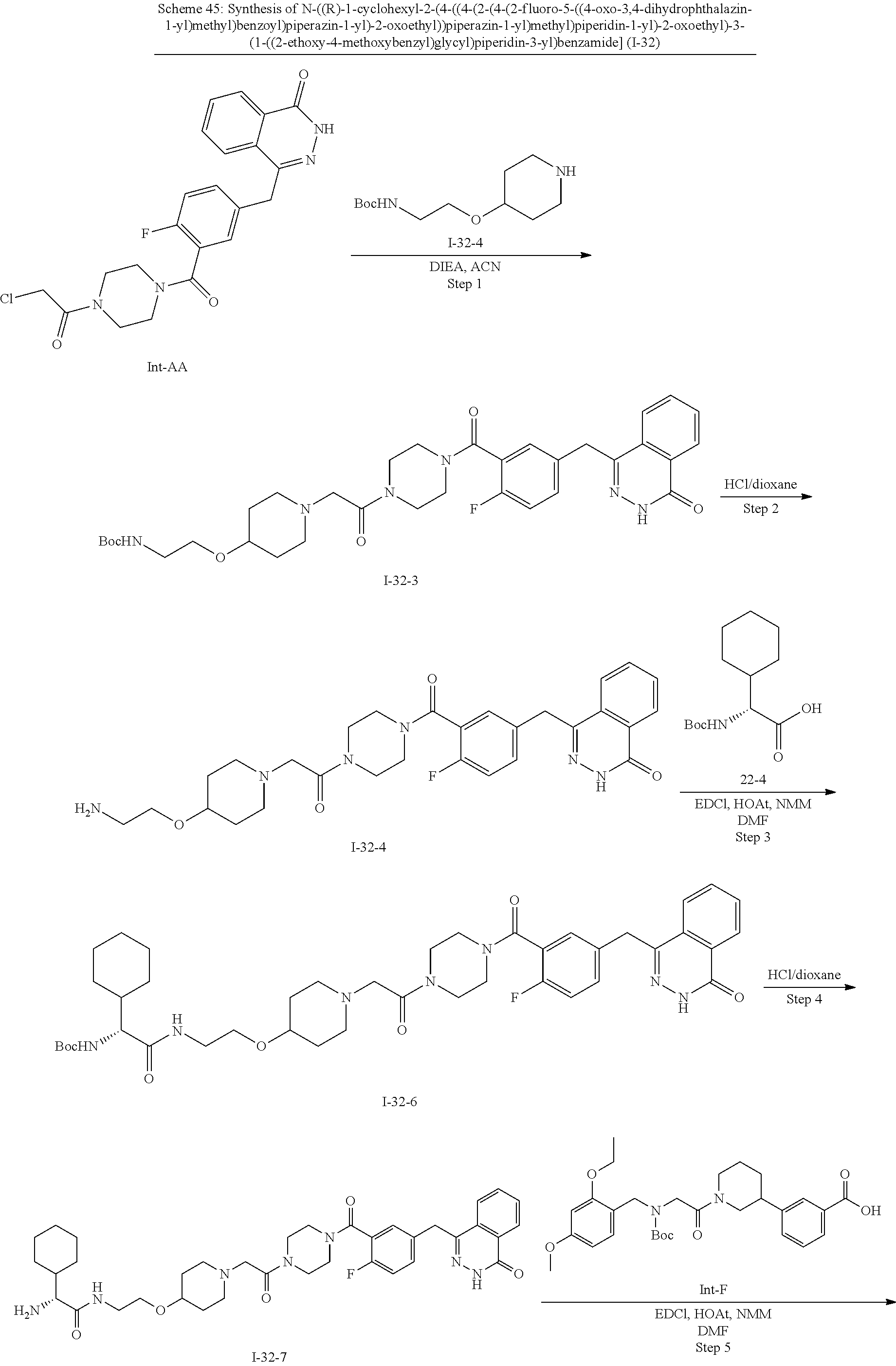

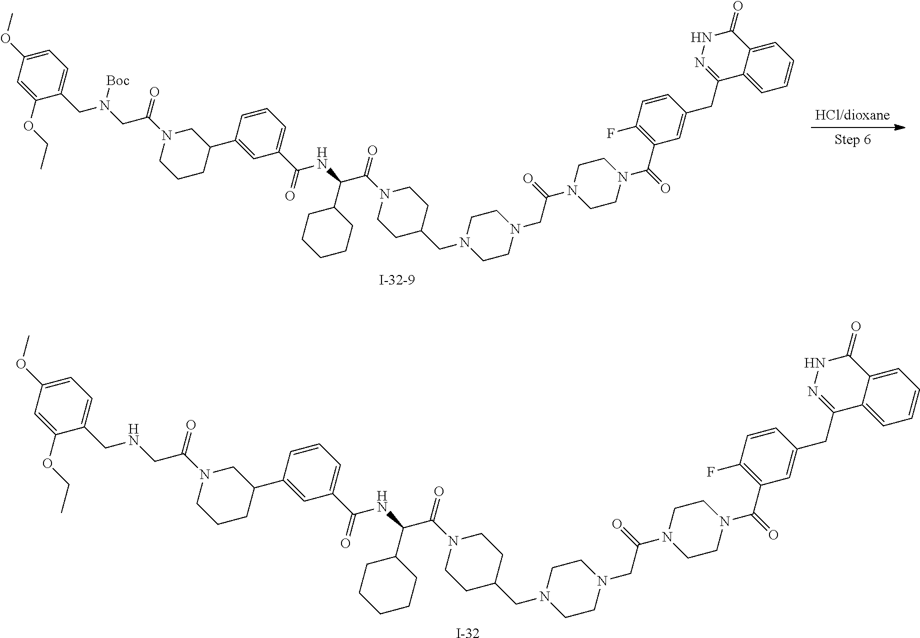

### Step 1: Synthesis of I-32-3

To a solution of Int-AA (500 mg, 1.13 mmol, 1 eq) and I-32-2 (275.85 mg, 1.13 mmol, 1 eq) in ACN (5 mL) was added DIEA (437.75 mg, 3.39 mmol, 589.95 μL, 3 eq). The mixture was stirred at 40° C. for 4 hr. The reaction mixture was concentrated under vacuum. The residue was purified by column chromatography (SiO₂, EA/MeOH=1/0 to 0/1, Rf=0.5, DCM:MeOH=10:1), and the eluent was concentrated under reduced pressure to give I-32-3 (490 mg, 655.10 μmol, 58.02% yield, 87% purity) as a brown oil. LCMS (Method D): Rt=0.365 min. ¹H NMR (400 MHz, methanol-d₄) δ 8.37 (d, J=7.6 Hz, 1H), 7.98-7.93 (m, 1H), 7.90-7.81 (m, 2H), 7.49 (s, 1H), 7.37 (s, 1H), 7.16-7.14 (m, 1H), 4.39 (s, 2H), 3.81-3.65 (m, 5H), 3.50-3.47 (m, 5H), 3.42 (d, J=1.2 Hz, 2H), 3.27-3.13 (m, 4H), 2.95-2.78 (m, 2H), 2.55-2.29 (m, 2H), 1.91 (s, 2H), 1.72-1.58 (m, 2H), 1.44 (s, 9H). ¹⁹F NMR (376 MHz, methanol-d₄) δ−120.792.

### Step 2: Synthesis of I-32-4

A solution of I-32-3 (380 mg, 583.95 μmol, 1 eq) in CH₂Cl₂ (3.80 mL) and HCl/dioxane (4 M, 3.80 mL, 26.03 eq) was stirred at 25° C. for 1 hour. The mixture was concentrated under reduced pressure to give I-32-4 (350 mg, crude, HCl) as a yellow solid, which was used in the next step without further purification. LCMS (Method D): Rt=0.233 min.

### Step 3: Synthesis of I-32-6

To a solution of I-32-4 (350 mg, 596.17 μmol, 1 eq, HCl) in DMF (4 mL) was added I-32-5 (184.09 mg, 715.40 μmol, 1.2 eq), HOAt (324.58 mg, 2.38 mmol, 333.58 μL, 4 eq), NMM (361.81 mg, 3.58 mmol, 393.28 μL, 6 eq) and EDCI (228.57 mg, 1.19 mmol, 2 eq). The mixture was stirred at 25° C. for 1 hr. The reaction mixture diluted with water (5 mL), extracted with EA (10 mL*2), and the combined organic layers were washed with brine (5 mL*2), dried over Na₂SO₄, filtered and concentrated under reduced pressure to give a residue. The crude product was purified by reversed-phase HPLC (0.10% FA condition) to give I-32-6 (240 mg, 284.38 μmol, 47.70% yield, 93.6% purity) as a yellow solid. LCMS (Method D): Rt=0.363 min. SFC (Method W): Rt=0.577 min, 0.699 min.

### Step 4: Synthesis of I-32-7

A solution of I-32-6 (240 mg, 303.82 μmol, 1 eq) in CH₂Cl₂ (2.4 mL) and HCl/dioxane (4 M, 2.4 mL, 31.60 eq) was stirred at 25° C. for 0.5 hr. The mixture was concentrated under reduced pressure to give I-32-7 (220 mg, 302.91 μmol, 99.70% yield, HCl) as a white solid, which was used in the next step without further purification. LCMS (Method D): Rt=0.299 min.

### Step 5: Synthesis of I-32-9

To a solution of I-32-7 (220 mg, 302.91 μmol, 1 eq, HCl) in DMF (2.5 mL) was added Int-F (191.42 mg, 363.50 μmol, 1.2 eq), HOAt (164.92 mg, 1.21 mmol, 169.50 μL, 4 eq), NMM (183.83 mg, 1.82 mmol, 199.82 μL, 6 eq) and EDCI (116.14 mg, 605.83 μmol, 2 eq). The mixture was stirred at 25° C. for 1 hr. The reaction mixture diluted with H₂O (3 mL) and extracted with EA (5 mL*2). The combined organic layers were washed with brine 10 mL (5 mL*2), dried over Na₂SO₄, filtered and concentrated under reduced pressure to give a residue. The crude product was purified by reversed-phase HPLC (0.1% FA condition) to give I-32-9 (200 mg, 158.88 μmol, 52.45% yield, 95.2% purity) as a white solid. LCMS (Method D): Rt=0.471 min.

### Step 6: Synthesis of I-32

A solution of I-32-9 (100 mg, 83.44 μmol, 1 eq) in CH₂Cl₂ (1 mL) and HCl/dioxane (4 M, 0.5 mL, 23.97 eq) was stirred at 25° C. for 0.2 hr. The mixture was concentrated to give a residue. The crude product was purified by reversed-phase HPLC (0.1% FA condition). The eluent was concentrated under reduced pressure to remove ACN and then lyophilized to give the crude product. The crude product was purified by reversed-phase HPLC (0.1% NH₃·H₂O condition) to give I-32 (19.34 mg, 17.56 μmol, 21.04% yield, 99.7% purity) as a white solid. LCMS (Method D): Rt=0.398 min. SFC (Method X): Rt=3.053 min, 4.701 min. ¹H NMR (400 MHz, methanol-d₄) δ 8.38-8.34 (m, 1H), 7.97-7.91 (m, 1H), 7.89-7.78 (m, 2H), 7.78-7.67 (m, 2H), 7.51-7.34 (m, 4H), 7.19-7.08 (m, 2H), 6.54-6.40 (m, 2H), 4.57-4.46 (m, 1H), 4.37 (s, 3H), 4.11-3.98 (m, 2H), 3.85-3.62 (m, 10H), 3.57-3.39 (m, 7H), 3.38-3.32 (m, 2H), 3.29-3.25 (m, 1H), 3.22-3.01 (m, 3H), 2.79-2.60 (m, 4H), 2.27-2.11 (m, 2H), 2.07-1.96 (m, 2H), 1.91-1.71 (m, 9H), 1.69-1.47 (m, 4H), 1.46-1.37 (m, 3H), 1.34-1.05 (m, 5H). ¹⁹F NMR (376 MHz, methanol-d₄) δ −120.741.

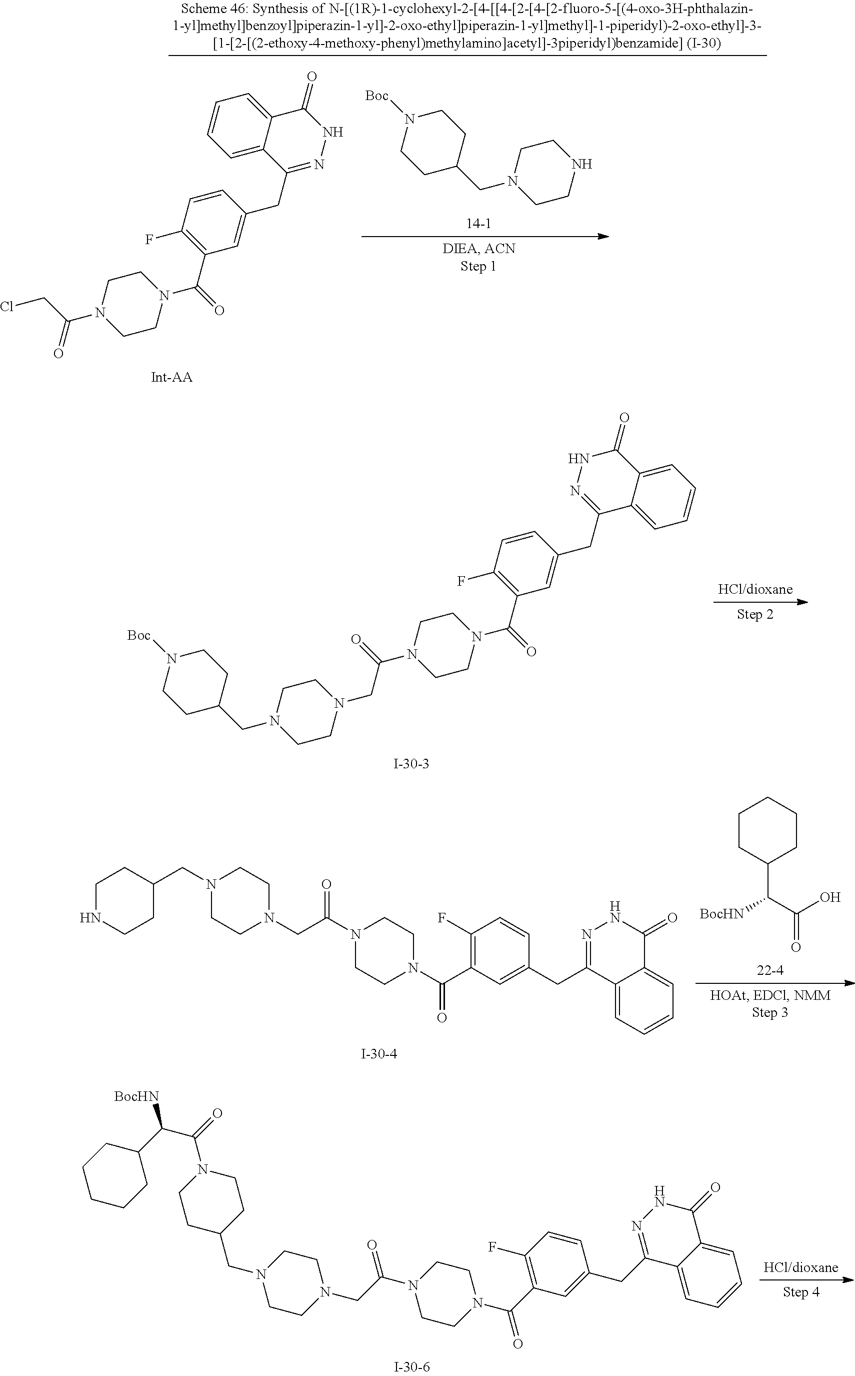 [SMILES: O=C(CCl)N1CCN(C(=O)c2cc(Cc3n[nH]c(=O)c4ccccc34)ccc2F)CC1 ; CC(C)(C)OC(=O)N1CCC(CN2CCNCC2)CC1 ; CC(C)(C)OC(=O)N1CCC(CN2CCN(CC(=O)N3CCN(C(=O)c4cc(Cc5n[nH]c(=O)c6ccccc56)ccc4F)CC3)CC2)CC1 ; O=C(CN1CCN(CC2CCNCC2)CC1)N1CCN(C(=O)c2cc(Cc3n[nH]c(=O)c4ccccc34)ccc2F)CC1 ; CC(C)(C)OC(=O)N[C@@H](C(=O)O)C1CCCCC1 ; CC(C)(C)OC(=O)N[C@@H](C(=O)N1CCC(CN2CCN(CC(=O)N3CCN(C(=O)c4cc(Cc5n[nH]c(=O)c6ccccc56)ccc4F)CC3)CC2)CC1)C1CCCCC1]

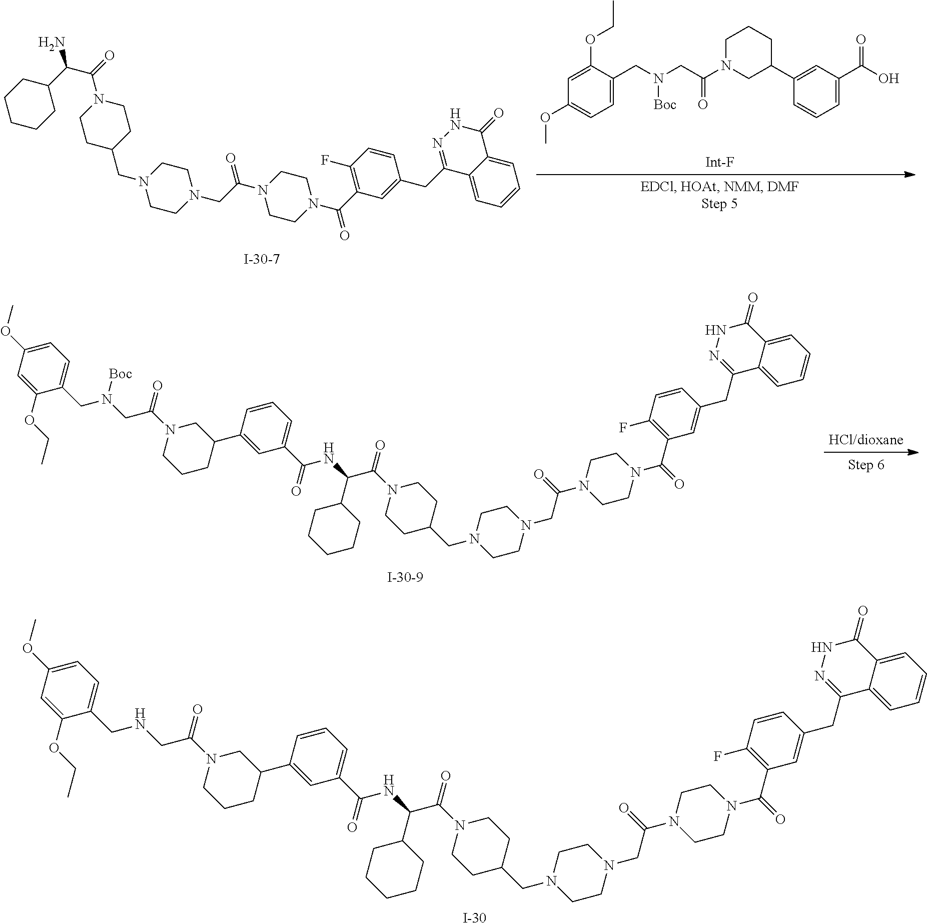

### Step 1: Synthesis of I-30-3

To a solution of 14-1 (95.99 mg, 338.70 μmol, 1.5 eq) in ACN (1 mL) was added Int-AA (100 mg, 225.80 μmol, 1 eq) and DIEA (87.55 mg, 677.40 μmol, 117.99 μL, 3 eq). The mixture was stirred at 25° C. for 4 hours. The mixture was concentrated under vacuum. The residue was purified by reversed-phase HPLC (0.1% FA) and the eluent was concentrated under reduced pressure to give I-30-3 (250 mg, 356.25 μmol, 78.89% yield, 98.3% purity) as a yellow solid. LCMS (Method D): Rt=0.325 min, [M+H]⁺=690.5. ¹H NMR (400 MHz, chloroform-d) δ 10.45-10.27 (m, 1H), 8.50-8.42 (m, 1H), 7.84-7.69 (m, 3H), 7.33 (s, 2H), 7.10-7.02 (m, 1H), 4.30 (s, 2H), 4.20-3.98 (m, 3H), 3.87-3.48 (m, 6H), 3.39-3.20 (m, 4H), 2.89-2.59 (m, 9H), 2.44 (s, 2H), 1.84-1.70 (m, 3H), 1.46 (s, 9H), 1.21-1.05 (m, 2H).

### Step 2: Synthesis of I-30-4

A solution of I-30-3 (250 mg, 362.41 μmol, 1 eq) in DCM (2.5 mL) and HCl/dioxane (4 M, 2.5 mL) was stirred at 25° C. for 2 hours. The mixture was concentrated under reduced pressure to give I-30-4 (320 mg, crude, HCl salt) as a yellow solid, which was used in the next step without further purification. LCMS (Method D): Rt=0.234 min, [M+H]⁺=590.6

### Step 3: Synthesis of I-30-6

To a solution of I-30-4 (220 mg, 351.35 μmol, 1 eq, HCl salt) in DMF (2.2 mL) was added I-30-5 (108.49 mg, 421.62 μmol, 1.2 eq), NMM (284.31 mg, 2.81 mmol, 309.03 μL, 8 eq), HOAt (239.11 mg, 1.76 mmol, 245.74 μL, 5 eq) and EDCI (134.71 mg, 702.69 μmol, 2 eq). The mixture was stirred at 25° C. for 1 hr. The reaction mixture diluted with water (10 mL) and extracted with EA (15 mL*2). The combined organic layers were washed with brine (10 mL*2), dried over Na₂SO₄, filtered and concentrated under reduced pressure to give a residue. The residue was purified by flash silica gel chromatography (20 g SepaFlash silica flash column; eluent: 0-15% MeOH/DCM at 50 mL/min). The eluent was concentrated under reduced pressure to give I-30-6 (210 mg, 253.31 μmol, 72.10% yield) as a yellow solid. LCMS (Method D): Rt=0.374 min, [M+H]⁺=829.6. SFC (Method K): Rt=2.401 min. ¹H NMR (400 MHz, chloroform-d) δ 11.02-10.87 (m, 1H), 8.51-8.42 (m, 1H), 7.86-7.69 (m, 3H), 7.41-7.29 (m, 2H), 7.10-7.00 (m, 1H), 5.48-5.36 (m, 1H), 5.15 (d, J=8.4 Hz, 1H), 4.58 (d, J=12.0 Hz, 1H), 4.46 (s, 1H), 4.30 (s, 2H), 4.22-4.14 (m, 1H), 3.97 (d, J=12.0 Hz, 1H), 3.87-3.63 (m, 4H), 3.59-3.53 (m, 2H), 3.09-2.99 (m, 4H), 2.71-2.54 (m, 8H), 2.33 (s, 2H), 1.76-1.71 (m, 4H), 1.66-1.61 (m, 4H), 1.44-1.41 (m, 9H), 1.25-1.22 (m, 2H), 1.16-1.14 (m, 2H), 1.13-1.10 (m, 2H), 1.07-1.00 (m, 2H).

### Step 4: Synthesis of I-30-7

To a solution of I-30-6 (210 mg, 253.31 μmol, 1 eq) in DCM (2.1 mL) was added HCl/dioxane (4 M, 2.1 mL, 33.16 eq). The mixture was stirred at 25° C. for 1 hour. The mixture was concentrated under reduced pressure to give I-30-7 (190 mg, 248.25 μmol, 98.00% yield, HCl salt) as a white solid and was used in the next step without further purification. LCMS (Method D): Rt=0.274 min, [M+H]⁺=729.6.

### Step 5: Synthesis of I-30-9

To a solution of I-30-7 (100 mg, 130.66 μmol, 1 eq, HCl salt) in DMF (1 mL) was added Int-F (82.57 mg, 156.79 μmol, 1.2 eq), HOAt (71.14 mg, 522.63 μmol, 73.11 μL, 4 eq), NMM (79.29 mg, 783.95 mol, 86.19 μL, 6 eq) and EDCI (50.09 mg, 261.32 μmol, 2 eq). The mixture was stirred at 25° C. for 1 hr. The reaction mixture diluted with H₂O (3 mL) and extracted with EA (5 mL*2). The combined organic layers were washed with brine (5 mL*2), dried over Na₂SO₄, filtered and concentrated under reduced pressure to give a residue. The residue was purified by reversed-phase HPLC (0.1% FA). The eluent was concentrated under reduced pressure to remove ACN and then lyophilized to give I-30-9 (50 mg, 39.76 μmol, 30.43% yield, 98.4% purity) as a yellow solid. LCMS (Method D): Rt=0.450 min, [M+H]⁺=1238.5.

### Step 6: Synthesis of I-30

To a solution of I-30-9 (70 mg, 56.57 μmol, 1 eq) in DCM (0.7 mL) was added HCl/dioxane (4 M, 0.35 mL, 24.75 eq). The mixture was stirred at 25° C. for 0.2 hr. To the mixture was added NH₃·H₂O to adjust the pH to 8. The mixture was concentrated under vacuum. The residue was purified by reversed-phase HPLC (neutral condition). The eluent was concentrated under reduced pressure to remove ACN and then lyophilized to give I-30 (15.58 mg, 13.10 μmol, 23.15% yield, 95.6% purity) as an off-white solid. LCMS: Rt=0.752 min, [M+H]⁺=1137.8. SFC (Method L): Rt=2.444 min, 2.739 min. ¹H NMR (400 MHz, methanol-d₄) δ 8.37 (d, J=7.6 Hz, 1H), 7.98-7.92 (m, 1H), 7.91-7.80 (m, 2H), 7.77-7.66 (m, 2H), 7.51-7.41 (m, 3H), 7.40-7.34 (m, 1H), 7.21-7.09 (m, 2H), 6.56-6.43 (m, 2H), 4.98-4.92 (m, 1H), 4.60-4.48 (m, 3H), 4.43-4.36 (m, 2H), 4.34-4.24 (m, 1H), 4.13-4.00 (m, 2H), 3.86-3.63 (m, 10H), 3.61-3.45 (m, 4H), 3.29-3.24 (m, 2H), 3.23-3.07 (m, 3H), 2.80-2.64 (m, 3H), 2.62-2.34 (m, 8H), 2.28-2.14 (m, 2H), 2.05-2.00 (m, 1H), 1.93-1.74 (m, 9H), 1.72-1.64 (m, 2H), 1.47-1.38 (m, 3H), 1.36-1.19 (m, 5H), 1.16-1.04 (m, 3H). ¹⁹F NMR (376 MHz, methanol-d₄) δ −120.73.

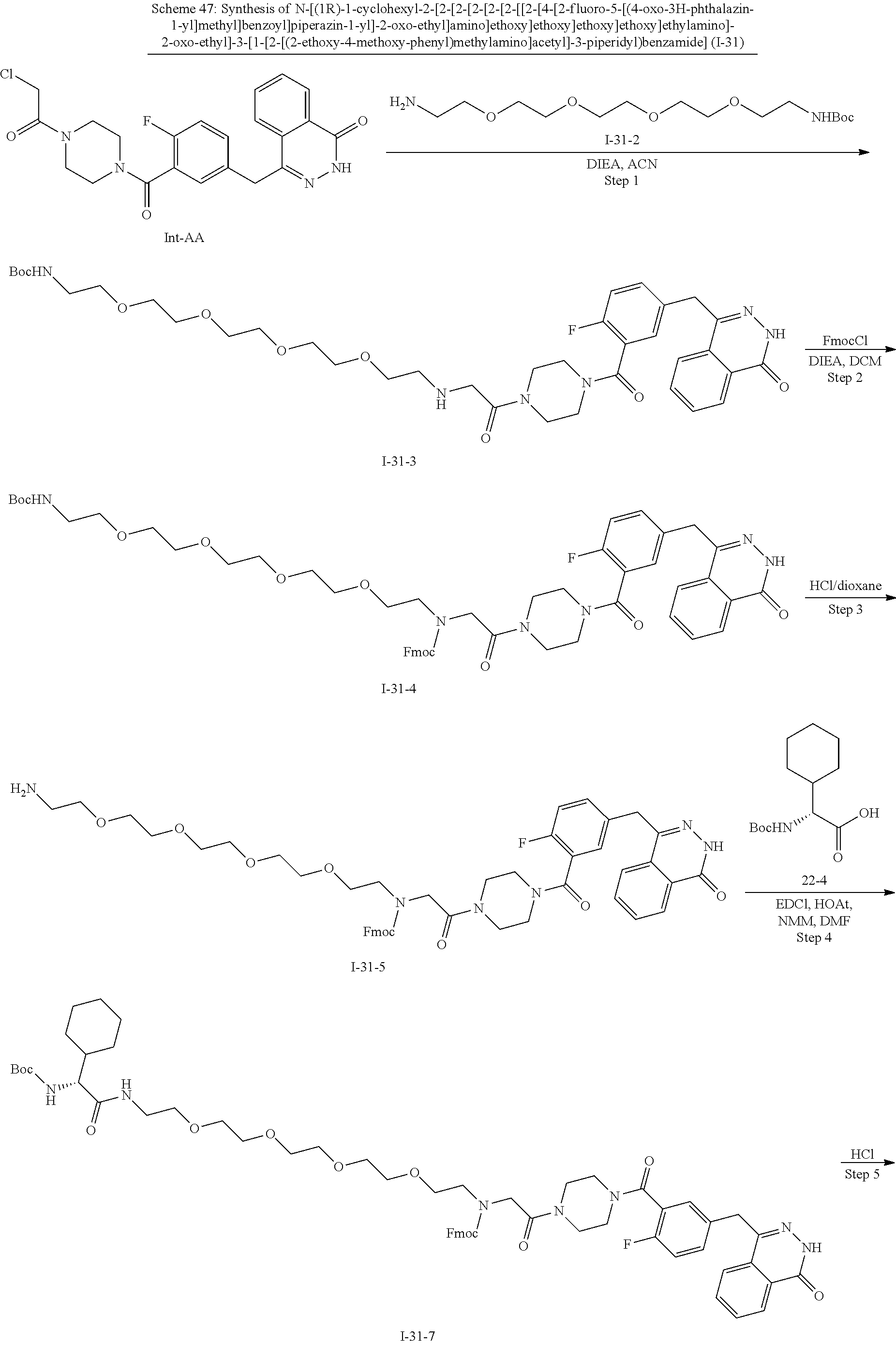

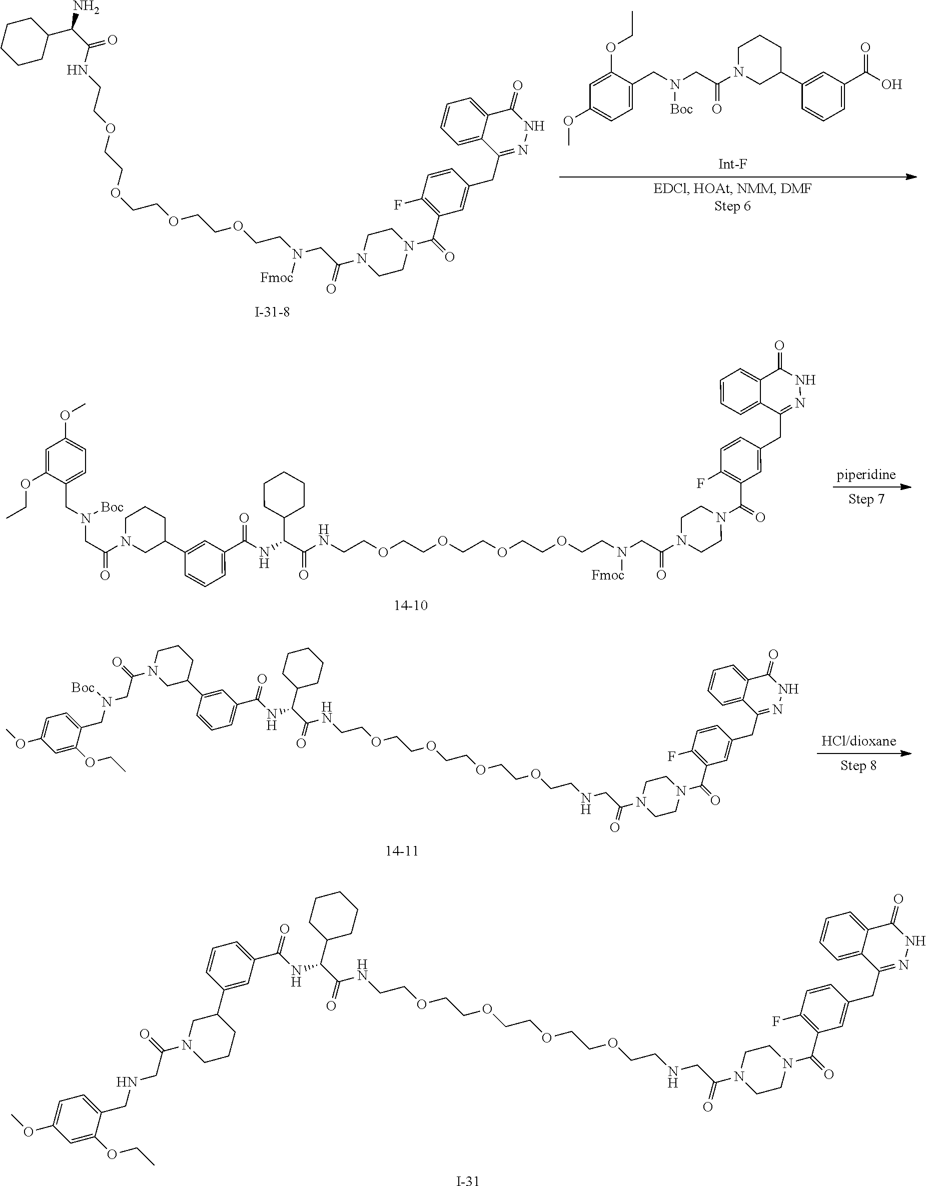

### Step 1: Synthesis of I-31-3

To a solution of Int-AA (500 mg, 1.13 mmol, 1 eq) and I-31-2 (455.79 mg, 1.35 mmol, 1.2 eq) in ACN (5 mL) was added DIEA (583.66 mg, 4.52 mmol, 786.61 μL, 4 eq). The mixture was stirred at 25° C. for 4 h. The mixture was concentrated under reduced pressure to give a residue. The crude product was purified by column chromatography (SiO₂, DCM/MeOH=1/0 to 0/1) and the eluent was concentrated under reduced pressure to give 1-31-3 (450 mg, 496.75 μmol, 44.00% yield, 82% purity) as a yellow solid. LCMS (Method D): Rt=0.352 min, [M+H]⁺=743.3.

### Step 2: Synthesis of I-31-4

To a solution of I-31-3 (450 mg, 605.79 μmol, 1 eq) in DCM (5 mL) was added DIEA (234.88 mg, 1.82 mmol, 316.55 μL, 3 eq) and Fmoc-Cl (188.06 mg, 726.95 μmol, 1.2 eq). The mixture was stirred at 25° C. for 12 h. The reaction mixture was diluted with H₂O (5 mL), extracted with DCM 15 ml (5 ml*3), dried with anhydrous sodium sulfate, filtered and concentrated to give I-31-4 (600 mg, crude) as a yellow solid. LCMS (Method D): Rt=0.504 min, [M+H]⁺=965.5

### Step 3: Synthesis of I-31-5

To a solution of I-31-4 (580 mg, 600.99 μmol, 1 eq) in dioxane (3 mL) was added HCl/dioxane (4 M, 3 mL, 19.97 eq). The mixture was stirred at 25° C. for 0.5 h. The mixture was concentrated under reduced pressure to give I-31-5 (550 mg, crude, HCl salt) as a yellow solid. LCMS (Method D): Rt=0.387 min, [M+H]⁺=865.5

### Step 4: Synthesis of I-31-7

To a solution of 22-4 (188.41 mg, 732.18 μmol, 1.2 eq) in DMF (6 mL) was added EDCI (350.90 mg, 1.83 mmol, 3 eq), NMM (308.57 mg, 3.05 mmol, 335.41 μL, 5 eq) and HOAt (83.05 mg, 610.15 μmol, 85.35 μL, 1 eq). The mixture was stirred at 25° C. for 0.5 h. Then I-31-5 (550 mg, 610.15 μmol, 1 eq, HCl salt) was added into the mixture and the mixture was stirred at 25° C. for 1 h. The reaction mixture was diluted with H₂O (10 mL), extracted with EA (10 ml*3), dried with anhydrous sodium sulfate, filtered and concentrated to give the crude product. The crude product was purified by column chromatography (SiO₂, DCM/MeOH=1/0 to 0/1) and the eluent was concentrated under reduced pressure to give I-31-7 (390 mg, 349.64 μmol, 57.30% yield, 99% purity) as a white solid. LCMS: Rt=0.527 min, [M+H]⁺=1104.6. SFC (Method M): Rt=0.599 min.

### Step 5: Synthesis of I-31-8

To a solution of I-31-7 (370 mg, 335.06 μmol, 1 eq) in dioxane (2 mL) was added HCl/dioxane (4 M, 2 mL, 23.88 eq). The mixture was stirred at 25° C. for 0.5 h. The mixture was concentrated under reduced pressure to give I-31-8 (370 mg, crude, HCl salt) as a white solid. LCMS: Rt=0.396 min, [M+H]⁺=1005.5.

### Step 6: Synthesis of I-31-10

To a solution of Int-F (224.69 mg, 426.67 μmol, 1.2 eq) in DMF (4 mL) was added EDCI (204.49 mg, 1.07 mmol, 3 eq), HOAt (48.40 mg, 355.56 μmol, 49.74 μL, 1 eq) and NMM (179.82 mg, 1.78 mmol, 195.46 μL, 5 eq). The mixture was stirred at 25° C. for 0.25 h. Then I-31-8 (370 mg, 355.56 μmol, 1 eq, HCl salt) was added into the mixture and the mixture was stirred at 25° C. for 1 h. The reaction mixture was diluted with H₂O (15 mL), extracted with EA (15 ml*3), dried with anhydrous sodium sulfate, filtered and concentrated to give I-31-10 (520 mg, crude) as a yellow oil. LCMS: Rt=0.588 min, [M+H]⁺=1513.3.

### Step 7: Synthesis of I-31-11

To a solution of I-31-10 (500 mg, 330.52 μmol, 1 eq) in DMF (5 mL) was added piperidine (42.21 mg, 495.78 μmol, 48.96 μL, 1.5 eq). The mixture was stirred at 25° C. for 1 h. The mixture was purified by prep-HPLC (0.1% FA) and the eluent was lyophilized to give I-31-11 (280 mg, 212.63 μmol, 64.33% yield, 98% purity) as a white solid. LCMS: Rt=0.470 min, [M+H]⁺=1290.8. SFC (Method N): Rt=0.650 min, 0.969 min.

### Step 8: Synthesis of I-31

To a solution of I-31-11 (100 mg, 77.49 μmol, 1 eq) in dioxane (1 mL) was added HCl/dioxane (4 M, 0.5 mL, 25.81 eq). The mixture was stirred at 25° C. for 0.5 h. The mixture was concentrated under reduced pressure to give a residue. The crude product was purified by prep-HPLC (0.1% FA) and the eluent was lyophilized to give I-31 (38.76 mg, 29.06 μmol, 37.50% yield, 92.705% purity, FA salt) as a white solid. LCMS: Rt=0.379 min, [M+H]⁺=1190.8. SFC (Method O): Rt=4.005 min, 6.556 min. ¹H NMR (400 MHz, methanol-d₄) δ 8.49 (s, 1H), 8.36 (d, J=7.6 Hz, 1H), 7.97-7.91 (m, 1H), 7.89-7.80 (m, 2H), 7.77-7.70 (m, 2H), 7.52-7.41 (m, 3H), 7.36 (t, J=6.8 Hz, 1H), 7.29-7.23 (m, 1H), 7.20-7.13 (m, 1H), 6.63-6.53 (m, 2H), 4.57-4.46 (m, 1H), 4.43-4.36 (m, 3H), 4.18-4.09 (m, 4H), 4.03-3.90 (m, 4H), 3.80 (d, J=4.8 Hz, 4H), 3.74 (d, J=3.2 Hz, 4H), 3.68-3.58 (m, 14H), 3.54 (d, J=4.0 Hz, 4H), 3.45-3.34 (m, 4H), 3.21-3.08 (m, 3H), 2.95-2.71 (m, 2H), 2.06-2.00 (m, 1H), 1.89-1.65 (m, 9H), 1.50-1.42 (m, 3H), 1.30-1.06 (m, 5H). ¹⁹F NMR (376 MHz, methanol-d₄) δ −120.688

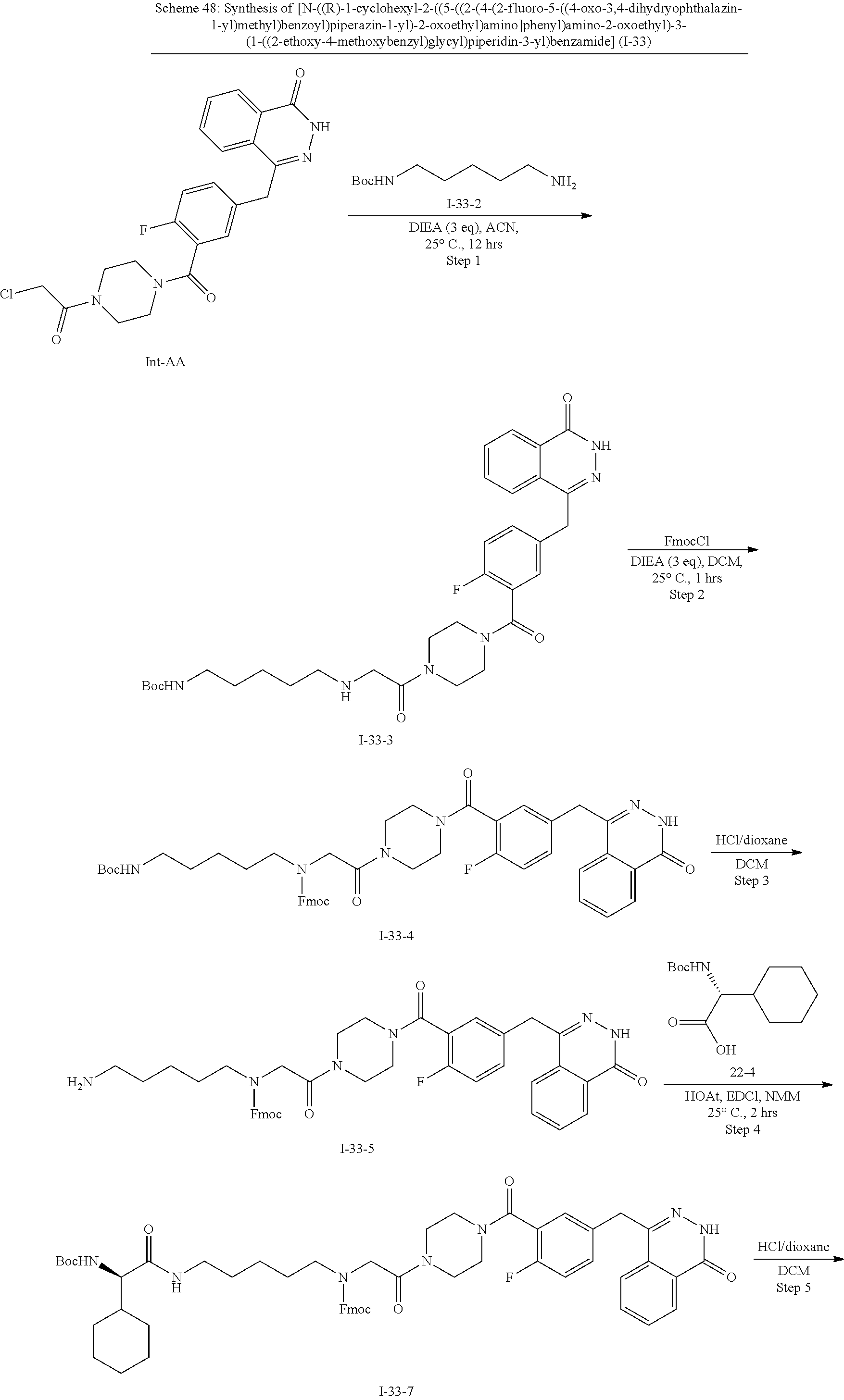

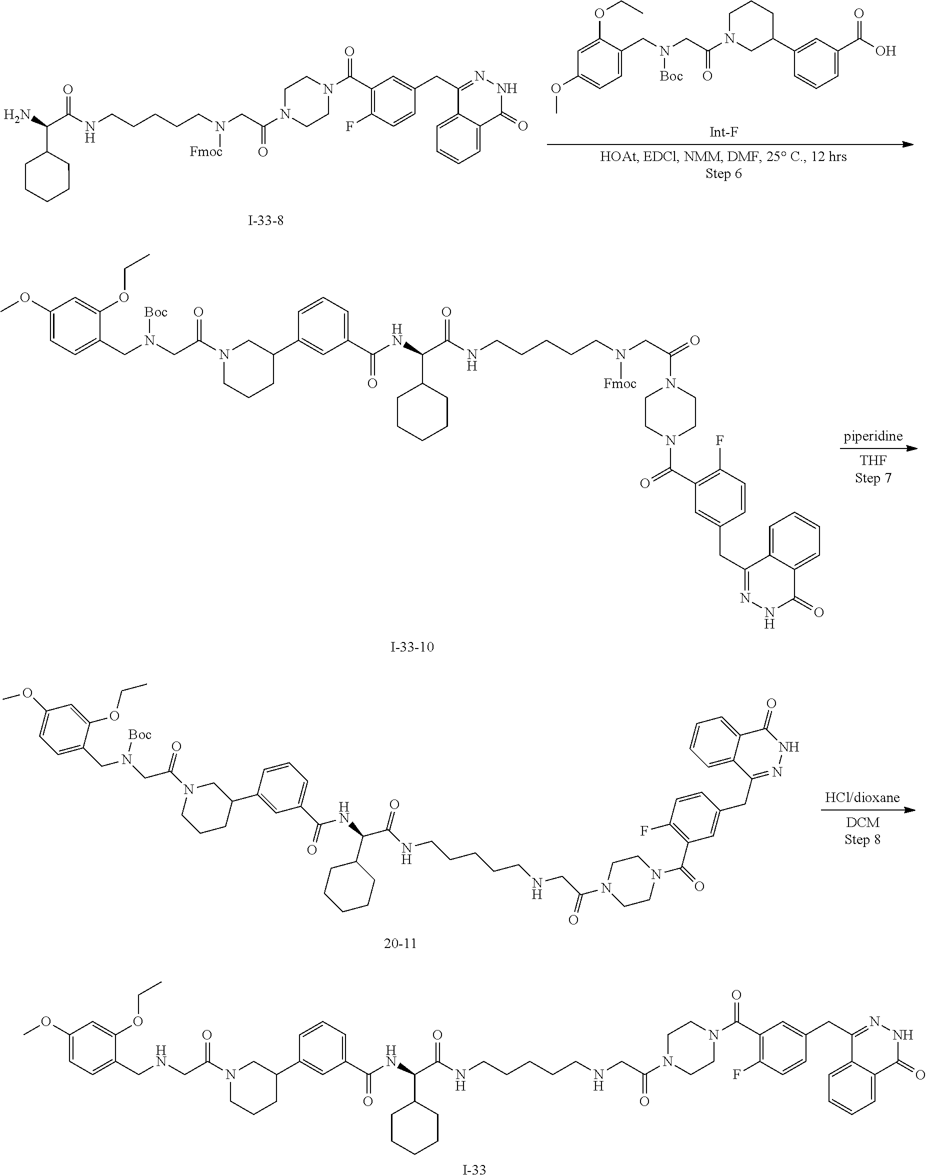

### Step 1: Synthesis of I-33-3

To a solution of Int-AA (200 mg, 451.60 μmol, 1 eq) and I-33-2 (137.03 mg, 677.40 μmol, 140.98 L, 1.5 eq) in ACN (2 mL) was added DIEA (175.10 mg, 1.35 mmol, 235.98 μL, 3 eq). The mixture was stirred at 25° C. for 1 hr. The reaction mixture was quenched by addition of H₂O (2 mL), and then extracted with EA (4 mL*2). The combined organic layers were washed with a saturated solution of NaCl (2 mL 2), dried over Na₂SO₄, filtered and concentrated under reduced pressure to give a residue. The residue was used in the next step without further purification. I-33-3 (270 mg, crude) was obtained as a yellow oil. LCMS (Method D): Rt=0.294 min, [M+H]⁺=609.4.

### Step 2: Synthesis of I-33-4

To a solution of I-33-3 (270 mg, 443.57 μmol, 1 eq) in DCM (3 mL) was added DIEA (171.98 mg, 1.33 mmol, 231.78 μL, 3 eq) and Fmoc-Cl (172.13 mg, 665.35 μmol, 1.5 eq). The mixture was stirred at 25° C. for 1 hr. The reaction mixture was quenched by addition H₂O (4 mL), and then extracted with EA (4 mL*3). The combined organic layers were dried over Na₂SO₄, filtered and concentrated under reduced pressure to give a residue. The residue was used in the next step without further purification. I-33-4 (270 mg, 292.44 μmol, 65.93% yield, 90% purity) was obtained as a yellow oil. LCMS (Method D): Rt=0.529 min, [M+H]⁺=831.5.

### Step 3: Synthesis of I-33-5

To a solution of I-33-4 (270 mg, 324.93 μmol, 1 eq) in DCM (2 mL) was added HCl/dioxane (2 mL). The mixture was stirred at 25° C. for 1 hr. The reaction mixture was concentrated under reduced pressure to give a residue. The residue was used in the next step without further purification. I-33-5 (250 mg, crude, HCl) was obtained as a white solid. LCMS (Method D): Rt=0.401 min, [M+H]⁺=731.4.

### Step 4: Synthesis of I-33-7

To a solution of 22-4 (250 mg, 325.82 μmol, 1 eq, HCl) in DMF (3 mL) was added EDCI (312.31 mg, 1.63 mmol, 5 eq), HOAt (88.70 mg, 651.65 μmol, 91.16 μL, 2 eq), NMM (329.56 mg, 3.26 mmol, 358.22 μL, 10 eq) and I-33-6 (83.84 mg, 325.82 μmol, 1 eq). The mixture was stirred at 25° C. for 1 hr. The reaction mixture was quenched by addition of H₂O (3 mL), and then extracted with EA (5 mL*2). The combined organic layers were washed with a saturated solution of NaCl (5 mL*2), dried over Na₂SO₄, filtered and concentrated under reduced pressure to give a residue. The residue was used in the next step without further purification. I-33-7 (300 mg, 265.63 μmol, 81.53% yield, 85.9% purity) was obtained as a yellow oil. LCMS (Method D): Rt=0.550 min, [M+H]⁺=970.5.

### Step 5: Synthesis of I-33-8

To a solution of I-33-7 (300 mg, 309.24 μmol, 1 eq) in DCM (2 mL) was added HCl/dioxane (2 mL). The mixture was stirred at 25° C. for 1 hr. The reaction mixture was concentrated under reduced pressure to give a residue. The residue was used in the next step without further purification. I-33-8 (300 mg, crude, HCl) was obtained as a yellow oil. LCMS (Method D): Rt=0.381 min, [M+H]⁺=870.5.

### Step 6: Synthesis of I-33-10

To a solution of I-33-8 (300 mg, 330.95 μmol, 1 eq, HCl) in DMF (3 mL) was added HOAt (90.09 mg, 661.90 μmol, 92.59 μL, 2 eq), EDCI (317.22 mg, 1.65 mmol, 5 eq), NMM (334.75 mg, 3.31 mmol, 363.85 μL, 10 eq) and Int-F (174.29 mg, 330.95 μmol, 1 eq). The mixture was stirred at 25° C. for 1 hr. The reaction mixture was quenched by addition of H₂O (3 mL), and then extracted with EA (5 mL*2). The combined organic layers were washed with a saturated solution of NaCl (5 mL*2), dried over Na₂SO₄, filtered and concentrated under reduced pressure to give a residue. The residue was used in the next step without further purification. I-33-10 (450 mg, crude) was obtained as a yellow oil. LCMS (Method D): Rt=0.631 min, [M+H]⁺=1378.9.

### Step 7: Synthesis of I-33-11

To a solution of I-33-10 (450 mg, 326.41 μmol, 1 eq) in THF (5 mL) was added piperidine (0.5 mL). The mixture was stirred at 25° C. for 0.5 hr. The reaction mixture was quenched by addition of H₂O (3 mL), and then extracted with EA (5 mL*2). The combined organic layers were washed with a saturated solution of NaCl (5 mL*2), dried over Na₂SO₄, filtered and concentrated under reduced pressure to give a residue. The residue was purified by flash silica gel chromatography (20 g SepaFlash silica flash column; eluent: 0-10% MeOH/DCM gradient at 20 mL/min) and the eluent was concentrated to give the product. I-33-11 (200 mg, 158.60 μmol, 48.59% yield, 91.7% purity) was obtained as a yellow oil. LCMS (Method D): Rt=0.493 min, [M+H]⁺=1156.7.

### Step 8: Synthesis of I-33

To a solution of I-33-11 (100 mg, 86.48 μmol, 1 eq) in DCM (1 mL) was added HCl/dioxane (0.5 mL). The mixture was stirred at 25° C. for 0.5 hr. The reaction mixture was quenched by addition of NH₃·H₂O (3 mL), and then concentrated under reduced pressure to give a residue. The residue was purified by prep-HPLC (FA condition) and the eluent was lyophilized to give the product. I-33 (17.53 mg, 16.35 μmol, 57.57% yield, 98.524% purity) was obtained as a white solid. LCMS (Method D): Rt=0.405 min, [M+H]⁺=1056.6. SFC (Method T): Rt=3.665 min, 7.433 min. ¹H NMR (400 MHz, methanol-d₄) δ 8.36 (d, J=7.6 Hz, 1H), 7.96-7.91 (m, 1H), 7.89-7.80 (m, 2H), 7.77-7.68 (m, 2H), 7.51-7.40 (m, 3H), 7.37 (s, 1H), 7.18-7.08 (m, 2H), 6.55-6.41 (m, 2H), 4.57-4.47 (m, 1H), 4.39-4.32 (m, 3H), 4.11-3.98 (m, 2H), 3.85-3.70 (m, 9H), 3.67 (d, J=2.0 Hz, 1H), 3.58-3.49 (m, 5H), 3.48-3.37 (m, 3H), 3.25-3.19 (m, 2H), 3.14-3.04 (m, 1H), 2.80-2.65 (m, 2H), 2.60 (d, J=7.2 Hz, 2H), 2.06-1.97 (m, 1H), 1.88-1.75 (m, 6H), 1.69 (d, J=1.6 Hz, 2H), 1.56 (s, 6H), 1.46-1.37 (m, 5H), 1.32-1.24 (m, 2H), 1.16-1.05 (m, 2H). ¹⁹F NMR (376 MHz, methanol-d₄) δ −120.680.

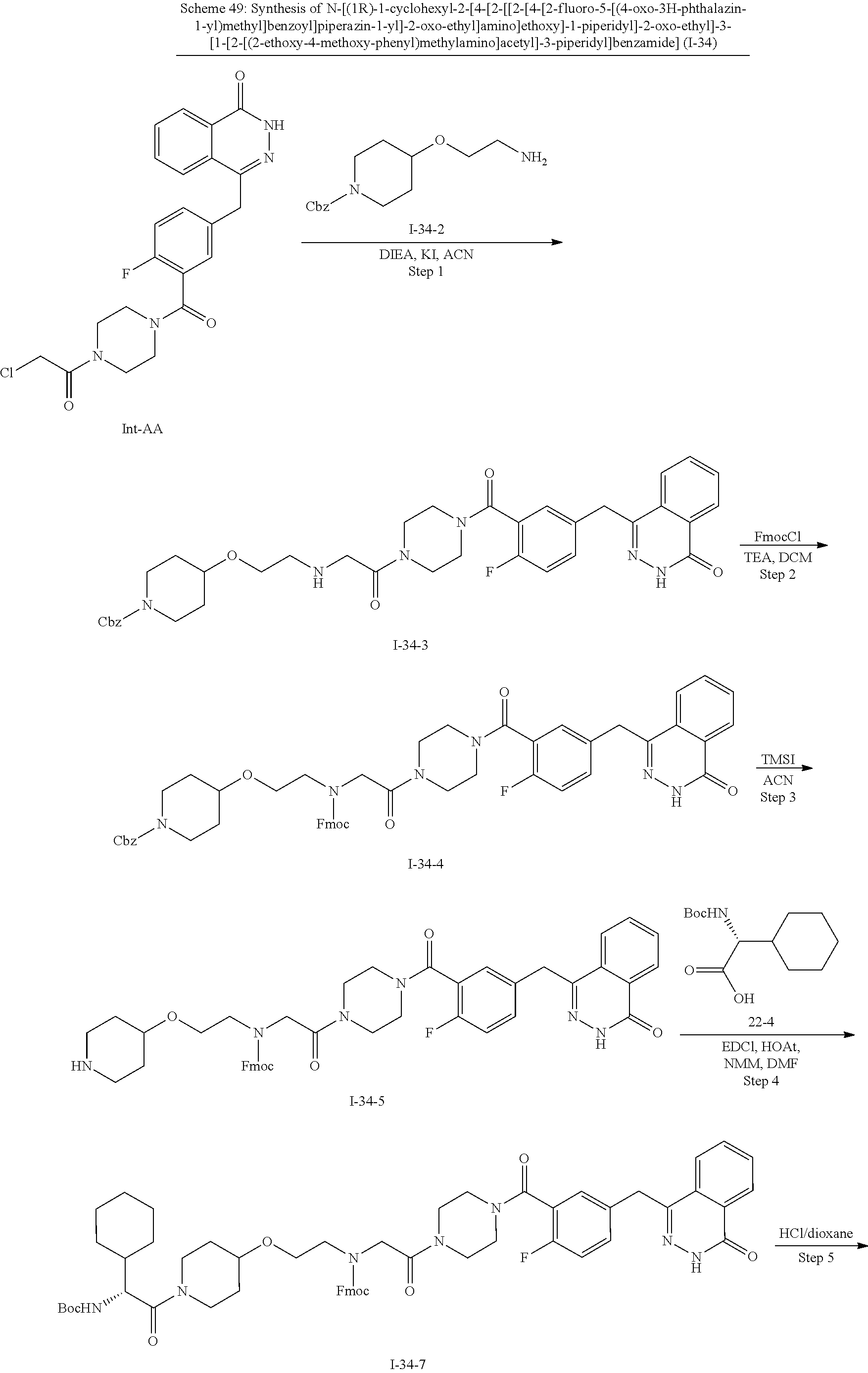

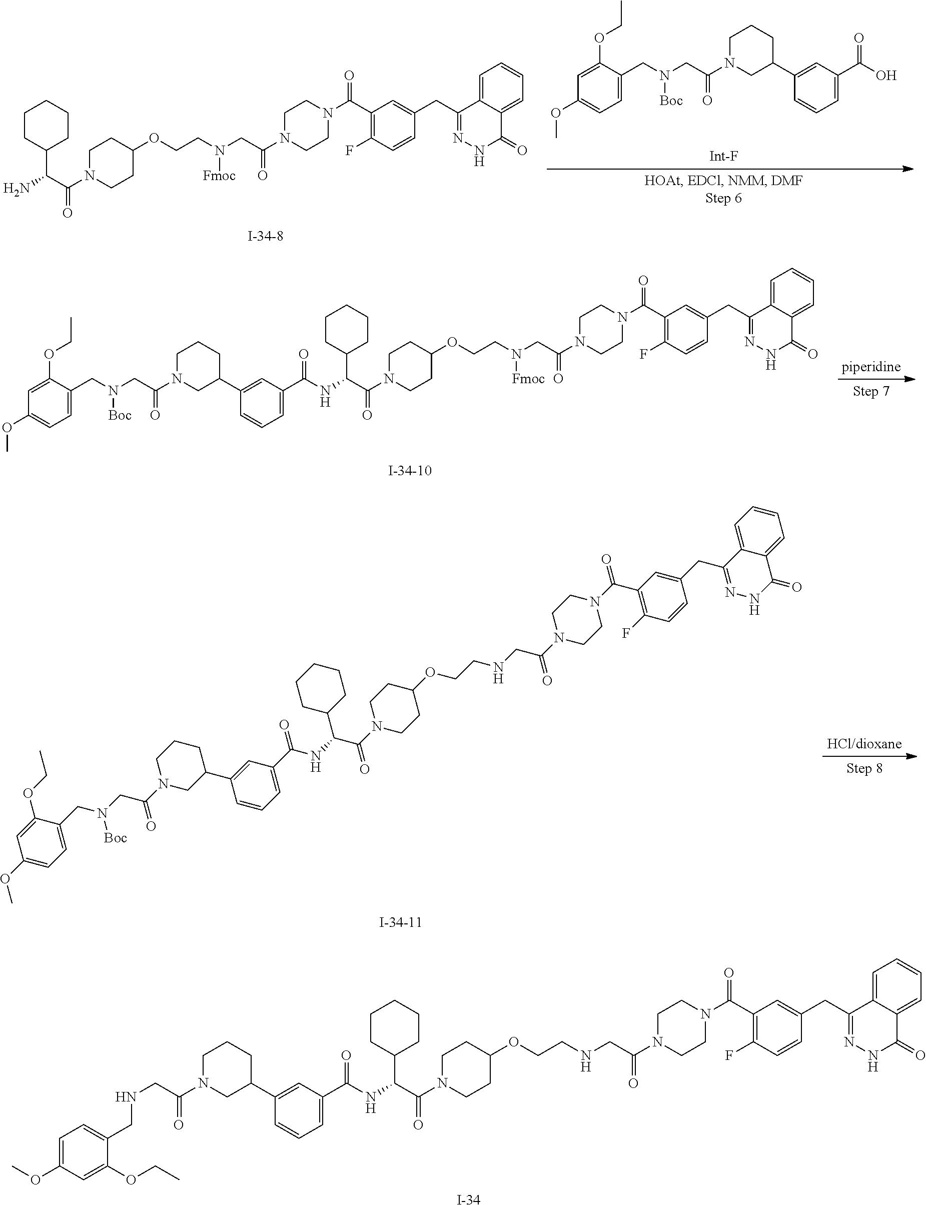

### Step 1: Synthesis of I-34-2

To a solution of Int-AA (500 mg, 1.13 mmol, 1 eq) and I-34-2 (314.25 mg, 1.13 mmol, 1 eq) in ACN (5 mL) was added DIEA (583.66 mg, 4.52 mmol, 786.61 μL, 4 eq) and KI (749.67 mg, 4.52 mmol, 4 eq). The mixture was stirred at 40° C. for 4 hr. The mixture was washed with water (10 mL) and extracted with DCM (15 mL*3). The combined organic layers were dried over sodium sulfate, filtered and concentrated under reduced pressure to give a residue. The residue was purified by column chromatography (SiO₂, EA/MeOH=1/0 to 0/1, Rf=0.5, DCM:MeOH=10:1) to give I-34-3 (1.4 g, 817.81 μmol, 72.44% yield, 40% purity) as a yellow gum. LCMS (Method D): Rt=0.394 min, [M+H]⁺=685.3.

### Step 2: Synthesis of I-34-4

To a solution of I-34-3 (1.2 g, 700.98 μmol, 1 eq, 40% purity) in DCM (12 mL) was added DIEA (271.78 mg, 2.10 mmol, 366.29 μL, 3 eq) and FmocCl (217.61 mg, 841.18 μmol, 1.2 eq). The mixture was stirred at 25° C. for 1 hr. The reaction mixture was concentrated under vacuum. The residue was purified by column chromatography (SiO₂, EA/MeOH=1/0 to 0/1, Rf=0.7, DCM:MeOH=10:1) to give I-34-4 (570 mg, 477.62 μmol, 68.14% yield, 76% purity) as a brown solid. LCMS (Method D): Rt=0.557 min, [M+H]+=907.5.

### Step 3: Synthesis of I-34-5

To a solution of I-34-4 (570 mg, 477.62 μmol, 1 eq, 76% purity) in ACN (6 mL) was added TMSI (573.41 mg, 2.87 mmol, 390.08 μL, 6 eq). The mixture was stirred at 0° C. for 0.5 hr. The reaction mixture was concentrated under vacuum. The residue was purified by prep-HPLC (FA condition) and the eluent was lyophilized to give I-34-5 (270 mg, 317.91 μmol, 66.56% yield, 91% purity) as a brown solid. LCMS (Method D): Rt=0.414 min, [M+H]⁺=773.3. ¹H NMR (400 MHz, methanol-d₄) δ 8.53 (s, 1H), 8.36 (d, J=7.6 Hz, 1H), 7.96-7.62 (m, 7H), 7.42-7.25 (m, 6H), 4.59 (s, 2H), 4.41-4.37 (m, 2H), 4.27-4.10 (m, 2H), 3.90-3.81 (m, 1H), 3.77-3.71 (m, 1H), 3.61 (d, J=13.6 Hz, 2H), 3.55-3.37 (m, 6H), 3.26 (d, J=12.4 Hz, 2H), 3.19 (s, 5H), 3.06 (d, J=2.0 Hz, 2H), 1.89 (d, J=11.2 Hz, 2H), 1.82-1.69 (m, 2H). ¹⁹F NMR (376 MHz, methanol-d₄) δ −120.687.

### Step 4: Synthesis of I-34-7

To a solution of I-34-5 (200 mg, 258.78 μmol, 1 eq) and 22-4 (66.59 mg, 258.78 μmol, 1 eq) in DMF (2 mL) was added EDCI (148.83 mg, 776.34 μmol, 3 eq), NMM (130.87 mg, 1.29 mmol, 142.25 μL, 5 eq) and HOAt (35.22 mg, 258.78 μmol, 36.20 μL, 1 eq). The mixture was stirred at 25° C. for 1 hr. The mixture was washed with water (5 mL) and extracted with EA (8 mL*3). The combined organic layers were dried over sodium sulfate, filtered and concentrated under reduced pressure to give a residue. The residue was purified by column chromatography (SiO₂, EA/MeOH=1/0 to 0/1, Rf=0.5, DCM:MeOH=10:1) to give I-34-7 (160 mg, 113.81 μmol, 43.98% yield, 72% purity) as a yellow oil. LCMS (Method D): Rt=0.542 min, [M+H]⁺=1012.7.

### Step 5: Synthesis of I-34-8

To a solution of I-34-7 (140 mg, 138.32 μmol, 1 eq, 72% purity) in DCM (1 mL) was added HCl/dioxane (4 M, 1.2 mL, 34.70 eq). The mixture was stirred at 25° C. for 0.5 hr. The reaction mixture was concentrated under reduced pressure to give I-34-8 (160 mg, crude, HCl salt) as a yellow solid. LCMS (Method D): Rt=0.413 min, [M+H]=912.4.

### Step 6: Synthesis of I-34-10

To a solution of I-34-8 (130 mg, 137.06 μmol, 1 eq, HCl salt) and Int-F (86.61 mg, 164.47 μmol, 1.2 eq) in DMF (1.5 mL) was added EDCI (78.82 mg, 411.17 μmol, 3 eq), NMM (69.31 mg, 685.28 μmol, 75.34 μL, 5 eq) and HOAt (18.65 mg, 137.06 μmol, 19.17 μL, 1 eq). The mixture was stirred at 25° C. for 1 hr. The mixture was washed with water (5 mL) and extracted with EA (8 mL*3). The combined organic layers were dried over sodium sulfate, filtered and concentrated under reduced pressure to give a residue. The residue was purified by column chromatography (SiO₂, EA/MeOH=1/0 to 0/1, Rf=0.5, DCM:MeOH=10:1) to give I-34-10 (140 mg, 98.55 μmol, 71.90% yield, 99% purity) as a yellow solid. LCMS (Method D): Rt=0.647 min, [M+H]⁺=1420.8.

### Step 7: Synthesis of I-34-11

To a solution of I-34-10 (140 mg, 98.55 μmol, 1 eq) in THF (1.6 mL) was added piperidine (172.44 mg, 2.03 mmol, 0.2 mL, 20.55 eq). The mixture was stirred at 25° C. for 0.5 hr. The reaction mixture was concentrated under vacuum. The residue was purified by column chromatography (SiO₂, DCM/MeOH=1/0 to 0/1, Rf=0.3, DCM:MeOH=10:1) to give I-34-11 (80 mg, 64.75 μmol, 65.71% yield, 97% purity) as a yellow solid. LCMS (Method D): Rt=0.495 min, [M+H]⁺=1198.6. SFC (Method Y): Rt=3.664 min, 4.716 min.

### Step 8: Synthesis of I-34

A solution of I-34-11 (45 mg, 37.55 μmol, 1 eq) in DCM (0.6 mL) was added HCl/dioxane (4 M, 300.00 μL, 31.96 eq). The mixture was stirred at 25° C. for 0.2 hr. The pH was acidified to 6-7 with NH₃·H₂O (0.3 ml) and the mixture was concentrated under vacuum. The residue was purified by prep-HPLC (0.1% NH₃·H₂O) and the eluent was lyophilized to give I-34 (16 mg, 14.06 μmol, 37.45% yield, 96.52% purity) as an off-white solid. LCMS (Method D): Rt=0.402 min, [M+H]⁺=1098.6. SFC (Method T): Rt=2.733 min, 3.364 min. ¹H NMR (400 MHz, methanol-d₄) δ 8.40-8.32 (m, 1H), 7.94 (d, J=7.2 Hz, 1H), 7.90-7.81 (m, 2H), 7.75-7.68 (m, 2H), 7.49-7.40 (m, 3H), 7.36 (d, J=10.4 Hz, 1H), 7.19-7.12 (m, 2H), 6.54-6.45 (m, 2H), 4.97-4.89 (m, 2H), 4.69-4.45 (m, 2H), 4.38 (d, J=7.2 Hz, 2H), 4.11-4.01 (m, 3H), 3.83-3.74 (m, 9H), 3.67-3.46 (m, 13H), 3.16-3.09 (m, 1H), 2.85-2.74 (m, 3H), 2.72-2.66 (m, 1H), 2.04-1.94 (m, 2H), 1.92-1.74 (m, 8H), 1.73-1.62 (m, 4H), 1.55 (s, 1H), 1.47-1.39 (m, 3H), 1.31-1.25 (m, 2H), 1.16-1.07 (m, 2H). ¹⁹F NMR (376 MHz, methanol-d₄) δ −120.778.

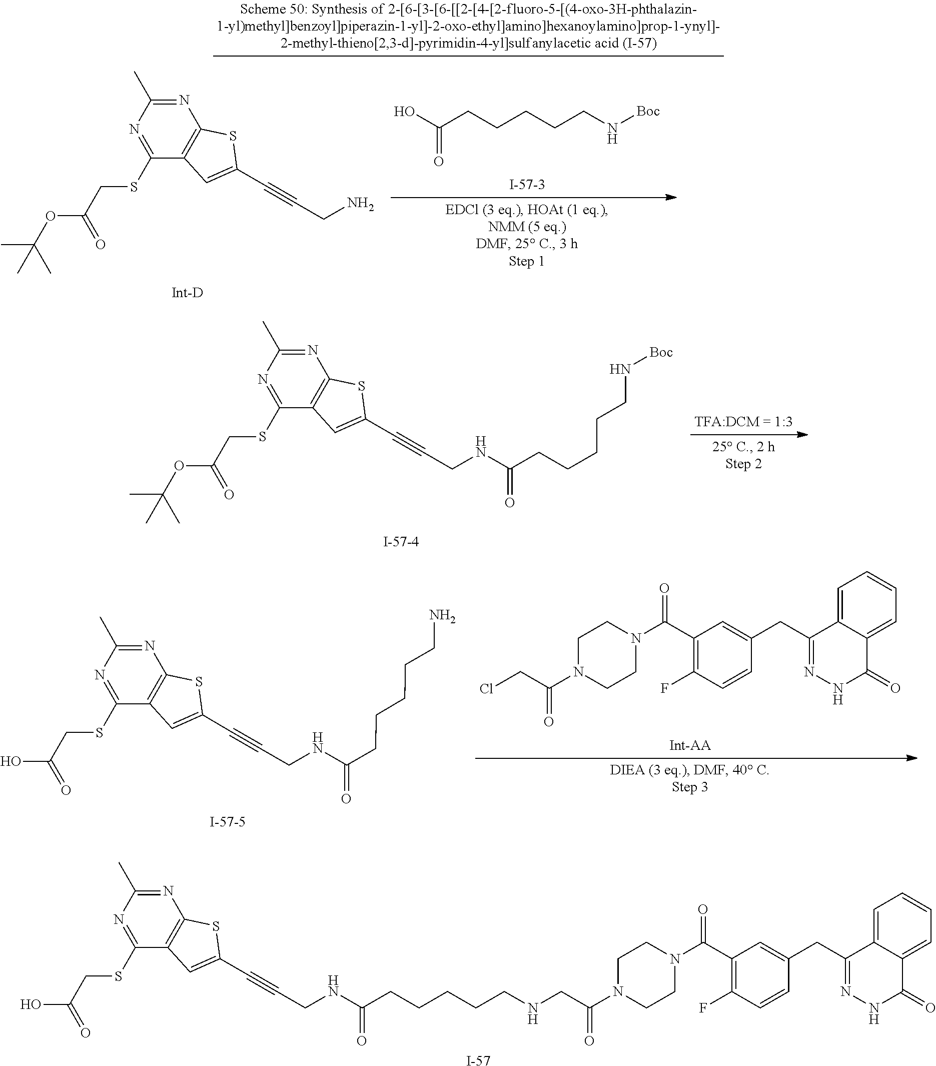

### Step 1: Synthesis of I-57-4

To a solution of Int-D (90 mg, 257.53 μmol, 1.2 eq), I-57-3 (49.64 mg, 214.61 μmol, 1 eq) and HOAt (29.21 mg, 214.61 μmol, 30.02 μL, 1 eq) in DMF (2 mL) was added EDCI (123.42 mg, 643.83 μmol, 3 eq) and NMM (108.54 mg, 1.07 mmol, 117.97 μL, 5 eq). The mixture was stirred at 25° C. for 2.5 hrs. The mixture was diluted with H₂O (8 mL) and extracted with EA (20 mL*3). The combined organic layers were washed with brine (15 mL*2), dried over anhydrous Na₂SO₄, filtered and concentrated under reduced pressure to give a residue. The residue was used for the next step without further purification. I-57-4 (90 mg, 140.76 μmol, 65.59% yield, 88.015% purity) was obtained as a yellow oil was obtained. LCMS (Method C): Rt=0.618 min, [M+H]⁺=563.3.

### Step 2: Synthesis of I-57-5

To a solution of I-57-4 (90 mg, 159.93 μmol, 1 eq) in DCM (1.5 mL) was added TFA (767.50 mg, 6.73 mmol, 0.5 mL, 42.09 eq). The mixture was stirred at 25° C. for 4.5 hrs. The reaction mixture was concentrated under reduced pressure to give I-57-5 (160 mg, crude) as a yellow oil, which was used in the next step without further purification. LCMS (Method E): Rt=0.394 min, [M+H]⁺=406.9.

### Step 3: Synthesis of I-57

To a solution of I-57-5 (65 mg, 159.89 μmol, 1 eq) in DMF (1 mL) was added DIEA (103.33 mg, 799.47 μmol, 139.25 μL, 5 eq) and Int-AA (63.73 mg, 143.90 μmol, 0.9 eq). The mixture was stirred at 40° C. for 16 hrs. The reaction mixture was diluted with DMF (1 mL) and filtered. The filtrate was purified by prep-HPLC (column: Phenomenex Luna C18 150 mm*25 mm*10 um; mobile phase: [water (TFA):ACN]; gradient: 20%-50% ACN over 9 min). I-57 (3.65 mg, 4.18 μmol, 2.61% yield, 93.079% purity) as a white solid was obtained. LCMS (Method E): Rt=0.440 min, [M+H]⁺=813.3. ¹H NMR (400 MHz, methanol-d₄) δ 8.36 (m, 1H), 7.97-7.91 (m, 1H), 7.90-7.79 (m, 2H), 7.54-7.47 (m, 1H), 7.47-7.42 (m, 1H), 7.40-7.34 (m, 1H), 7.20-7.12 (m, 1H), 4.38 (s, 2H), 4.34-4.28 (m, 2H), 4.28-4.23 (m, 2H), 3.86-3.61 (m, 4H), 3.60-3.50 (m, 2H), 3.49-3.38 (m, 2H), 3.37-3.33 (m, 2H), 2.92 (m, 2H), 2.71-2.62 (m, 3H), 2.34-2.23 (m, 2H), 1.74-1.62 (m, 4H), 1.48-1.38 (m, 2H).

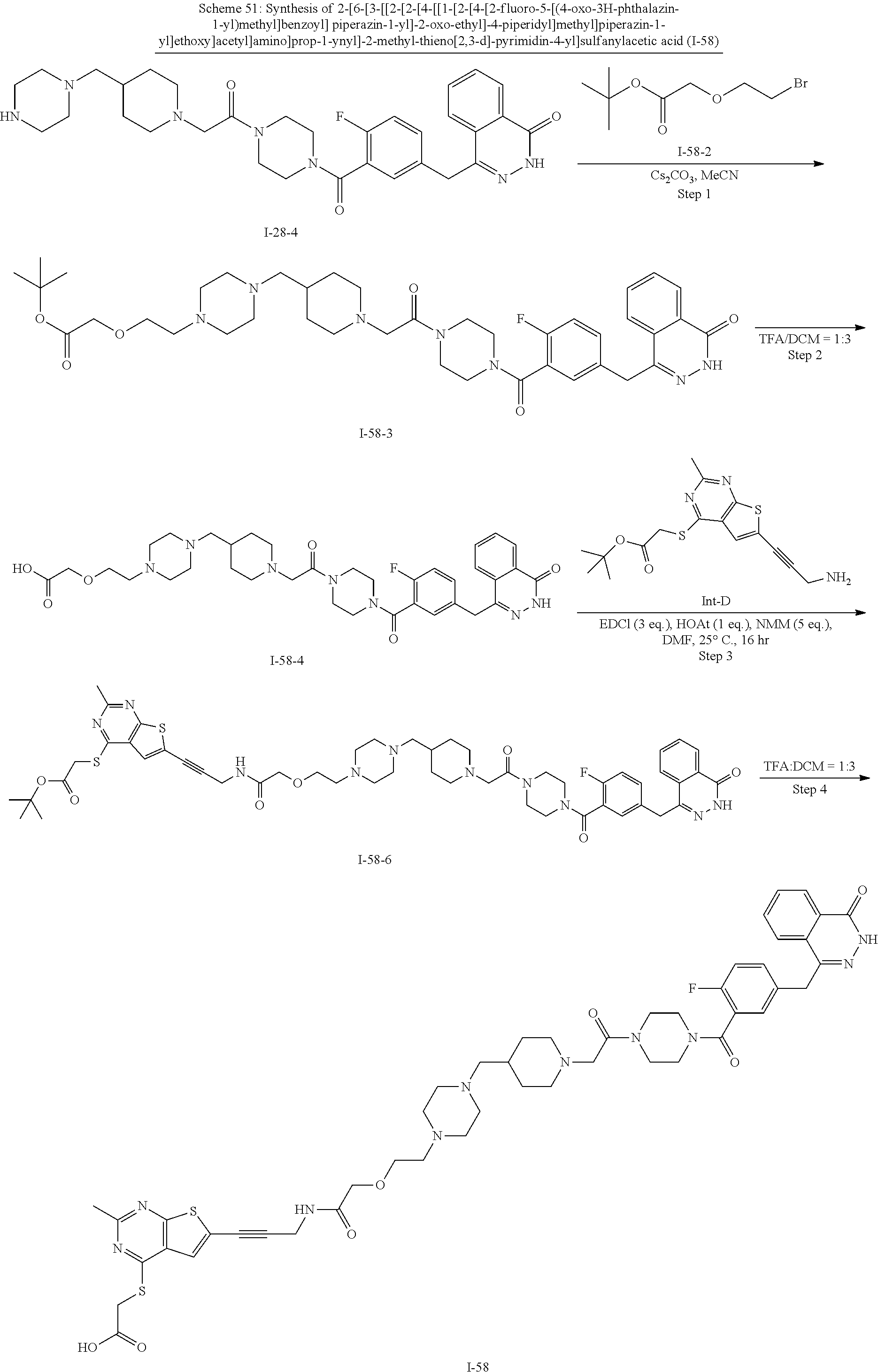

### Step 1: Synthesis of I-58-3

To a solution of I-28-4 (200 mg, 339.15 μmol, 1 eq) in CH₃CN (4 mL) was added I-58-2 (162.19 mg, 678.31 μmol, 2 eq) and Cs₂CO₃ (221.01 mg, 678.31 μmol, 2 eq). The mixture was stirred at 80° C. for 16 hrs. The reaction mixture was filtered and the filtrate was concentrated under reduced pressure to give a residue. The crude product was purified by reversed-phase HPLC (0.1% NH₃·H₂O). I-58-3 (140 mg, 161.13 μmol, 47.51% yield, 86.8% purity) was obtained as a yellow oil. LCMS (Method E): Rt=0.388 min, (M−H)=748.4.

### Step 2: Synthesis of I-58-4

To a solution of I-58-3 (100 mg, 133.71 μmol, 1 eq) in DCM (1.5 mL) was added TFA (767.50 mg, 6.73 mmol, 0.5 mL, 50.34 eq). The mixture was stirred at 25° C. for 2 hrs. The reaction mixture was concentrated under reduced pressure to give the crude product. The residue was used for the next step without further purification. I-58-4 (200 mg, crude) was obtained as a yellow oil. LCMS (Method E): Rt=0.349 min, [M+H]⁺=692.3.

### Step 3: Synthesis of I-58-6

To a solution of I-58-4 (200 mg, 289.10 μmol, 1 eq), Int-D (101.03 mg, 289.10 μmol, 1 eq) in DMF (1.5 mL) was added EDCI (166.27 mg, 867.31 μmol, 3 eq), HOAt (39.35 mg, 289.10 μmol, 40.44 μL, 1 eq) and NMM (146.21 mg, 1.45 mmol, 158.92 μL, 5 eq). The mixture was stirred at 25° C. for 2 hrs. The reaction mixture was filtered without a work up. The reaction mixture was purified by reversed-phase HPLC (0.1% NH₃·H₂O). I-58-6 (25 mg, crude) was obtained as a colorless oil. LCMS (Method C): Rt=0.963 min, [M+H]⁺=1023.6.

### Step 4: Synthesis of I-58

To a solution of I-58-6 (25 mg, 24.43 μmol, 1 eq) in DCM (0.3 mL) was added TFA (153.50 mg, 1.35 mmol, 0.1 mL, 55.10 eq). The mixture was stirred at 25° C. for 1.5 hr. The reaction mixture was filtered and concentrated under reduced pressure to give a residue. The residue was purified by prep-HPLC (column: Phenomenex Luna C18 150 mm*25 mm*10 um; mobile phase: [water (TFA):ACN]; gradient: 10%-40% ACN over 9 min). I-58 (8.53 mg, 8.73 μmol, 35.73% yield, 98.979% purity) was obtained as a white solid. LCMS (Method E): Rt=0.408 min, [M+H]⁺=967.4. ¹H NMR (400 MHz, methanol-d₄) δ 8.39-8.35 (m, 1H), 7.98-7.93 (m, 1H), 7.90-7.81 (m, 2H), 7.55-7.49 (m, 1H), 7.48 (s, 1H), 7.40-7.33 (m, 1H), 7.18 (m, 1H), 4.39 (s, 2H), 4.34 (s, 2H), 4.27 (br s, 1H), 4.19 (br s, 1H), 4.17 (m, 4H), 3.92-3.86 (m, 2H), 3.83-3.76 (m, 2H), 3.76-3.43 (m, 8H), 3.42-3.32 (m, 9H), 3.10-2.98 (m, 1H), 3.08-2.97 (m, 1H), 2.94-2.75 (m, 2H), 2.68 (s, 3H), 2.46-2.36 (m, 2H), 2.09-1.98 (m, 2H), 1.97-1.77 (m, 1H), 1.54 (br s, 1H).

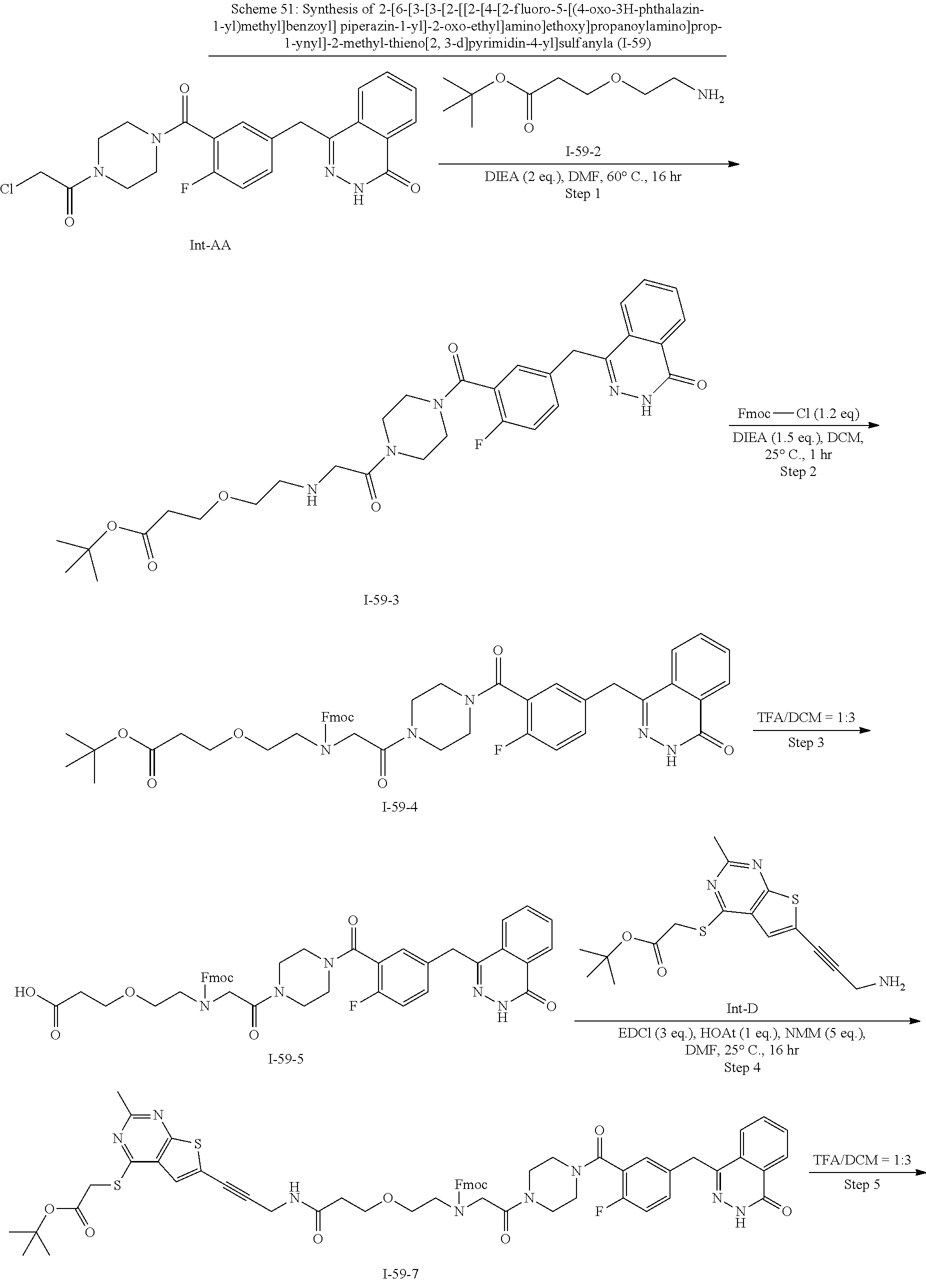

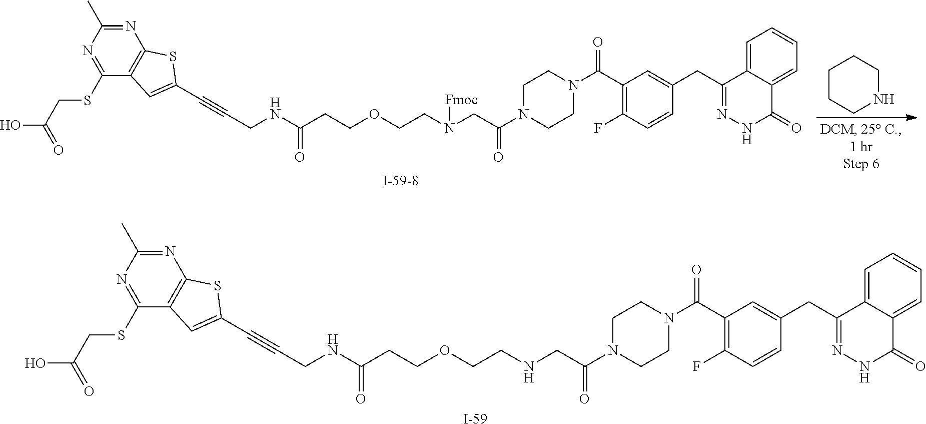

### Step 1: Synthesis of I-59-3

To a solution of Int-AA (500 mg, 1.13 mmol, 1 eq) in DMF (5 mL) was added I-59-2 (213.66 mg, 1.13 mmol, 1 eq) and DIEA (291.83 mg, 2.26 mmol, 393.30 μL, 2 eq). The mixture was stirred at 60° C. for 4 hrs. The mixture was diluted with H₂O (20 mL) and extracted with EA (20 mL*3). The combined organic layers were washed with brine (15 mL*2), dried over anhydrous Na₂SO₄, filtered and concentrated under reduced pressure to give a residue. The residue was purified by prep-HPLC (column: Waters Xbridge Prep OBD C18 150 mm*40 mm*10 um; mobile phase: [water (NH₄HCO₃):ACN]; gradient: 17%-47% ACN over 15 min). I-59-3 (150 mg, crude) as a yellow oil was obtained. LCMS (Method C): Rt=0.736 min, [M+H]⁺=596.5.

### Step 2: Synthesis of I-59-4

To a solution of I-59-3 (150 mg, 251.82 μmol, 1 eq) in DCM (1.5 mL) was added DIPEA (48.82 mg, 377.73 μmol, 65.79 μL, 1.5 eq) and Fmoc-Cl (78.18 mg, 302.19 μmol, 1.2 eq). The mixture was stirred at 25° C. for 2 hrs. The reaction mixture was filtered and concentrated under reduced pressure to give a residue. The residue was purified by flash silica gel chromatography (12 g SepaFlash silica flash column; eluent: 10% methanol/dichloromethane at 30 mL/min). I-59-4 (150 mg, crude) was obtained as a white solid. LCMS (Method E): Rt=0.592 min, [M+H]⁺=818.5.

### Step 3: Synthesis of I-59-5

To a solution of I-59-4 (150 mg, 183.40 μmol, 1 eq) in DCM (1.2 mL) was added TFA (614.00 mg, 5.38 mmol, 400.00 μL, 29.36 eq). The mixture was stirred at 25° C. for 1 hr. The reaction mixture was concentrated under reduced pressure to give I-59-5 (200 mg, crude) as brown oil, which was used in the next step without further purification. LCMS (Method C): Rt=0.558 min, [M+H]=762.4.

### Step 4: Synthesis of I-59-7

To a solution of I-59-5 (200 mg, 262.54 μmol, 1 eq), Int-D (91.75 mg, 262.54 μmol, 1 eq) in DMF (1.5 mL) was added EDCI (150.99 mg, 787.62 μmol, 3 eq), HOAt (35.73 mg, 262.54 μmol, 36.73 μL, 1 eq) and NMM (132.78 mg, 1.31 mmol, 144.32 μL, 5 eq). The mixture was stirred at 25° C. for 16 hrs. The mixture was diluted with H₂O (5 mL) and extracted with EA (15 mL*3). The combined organic layers were washed with brine (10 mL*2), dried over anhydrous Na₂SO₄, filtered and concentrated under reduced pressure to give a residue. The residue was purified by flash silica gel chromatography (12 g SepaFlash silica flash column; eluent: 8% methanol/dichloromethane gradient at 25 mL/min). I-59-7 (45 mg, crude) as brown oil was obtained. LCMS (Method E): Rt=0.629 min, [M+H]⁺=1093.5.

### Step 5: Synthesis of I-59-8

To a solution of I-59-7 (25 mg, 22.87 μmol, 1 eq) in DCM (0.3 mL) was added TFA (153.50 mg, 1.35 mmol, 0.1 mL, 58.87 eq). The mixture was stirred at 25° C. for 2 hrs. The reaction mixture was concentrated under reduced pressure to give the crude product. The residue was used for the next step without further purification. I-59-8 (50 mg, crude) was obtained as brown oil. LCMS (Method C): Rt=0.583 min, [M+H]⁺=1037.5.

### Step 6: Synthesis of I-59

To a solution of I-59-8 (50 mg, 48.21 μmol, 1 eq) in DCM (0.4 mL) was added piperidine (41.05 mg, 482.09 μmol, 47.61 μL, 10 eq). The mixture was stirred at 25° C. for 1 hr. The reaction mixture was concentrated under reduced pressure to give the crude product. The residue was purified by prep-HPLC (column: Phenomenex Luna C18 150 mm*25 mm*10 um; mobile phase: [water (TFA):ACN]; gradient: 16%-46% ACN over 9 min). I-59 (9.71 mg, 11.92 μmol, 24.72% yield, 100% purity) as a yellow solid was detected. LCMS (Method E): Rt=0.422 min, [M+H]⁺=815.2. ¹H NMR (400 MHz, methanol-d₄) δ 8.40-8.29 (m, 1H), 7.96-7.89 (m, 1H), 7.88-7.77 (m, 2H), 7.53-7.46 (m, 1H), 7.45-7.31 (m, 2H), 7.20-7.09 (m, 1H), 4.36 (m, 2H), 4.29 (s, 1H), 4.25-4.20 (m, 1H), 4.19-4.08 (m, 4H), 3.86-3.74 (m, 6H), 3.74-3.68 (m, 1H), 3.54 (m, 2H), 3.41 (s, 2H), 3.29 (br s, 3H), 2.66 (m, 3H), 2.59-2.53 (m, 2H).

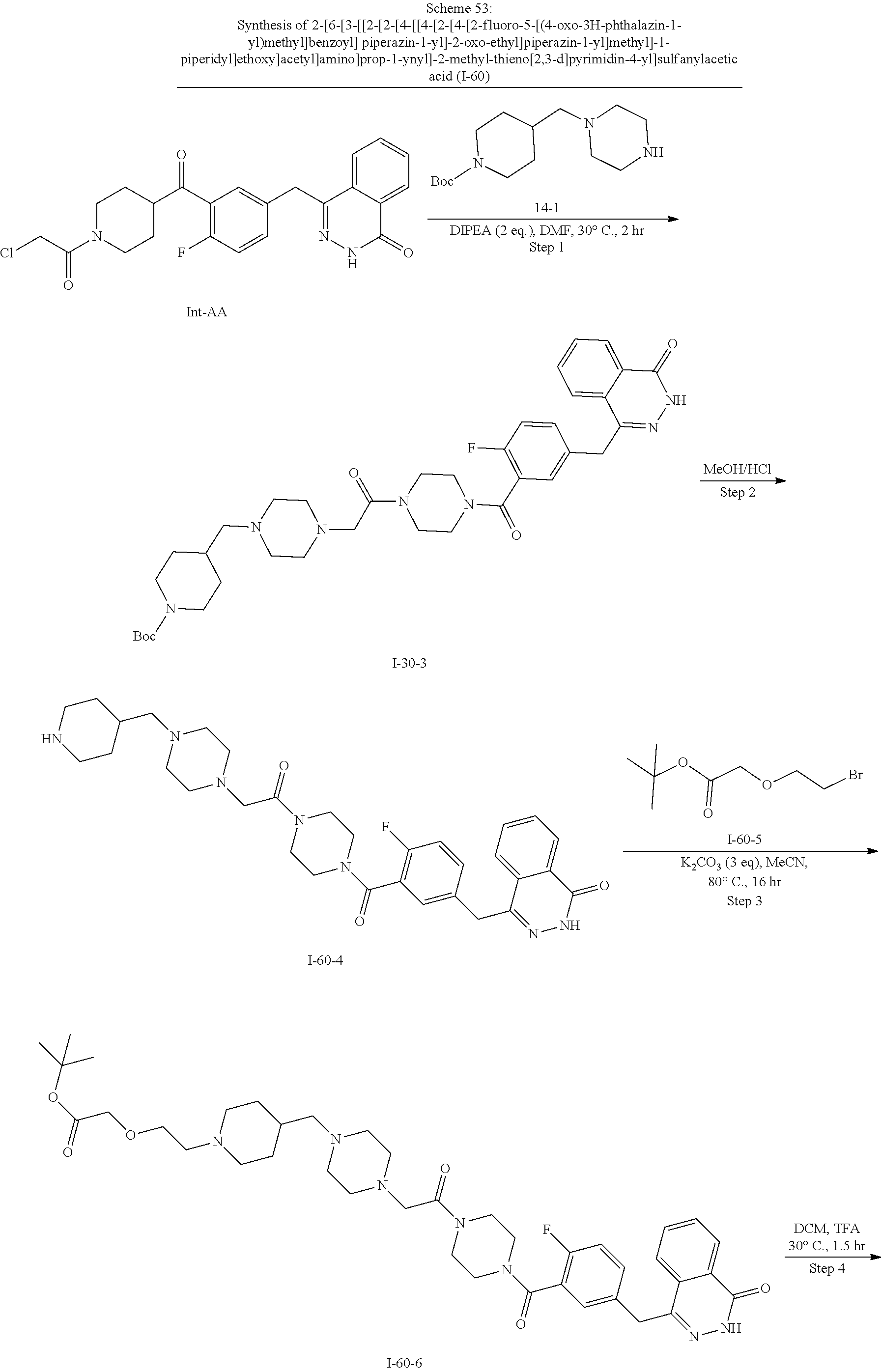

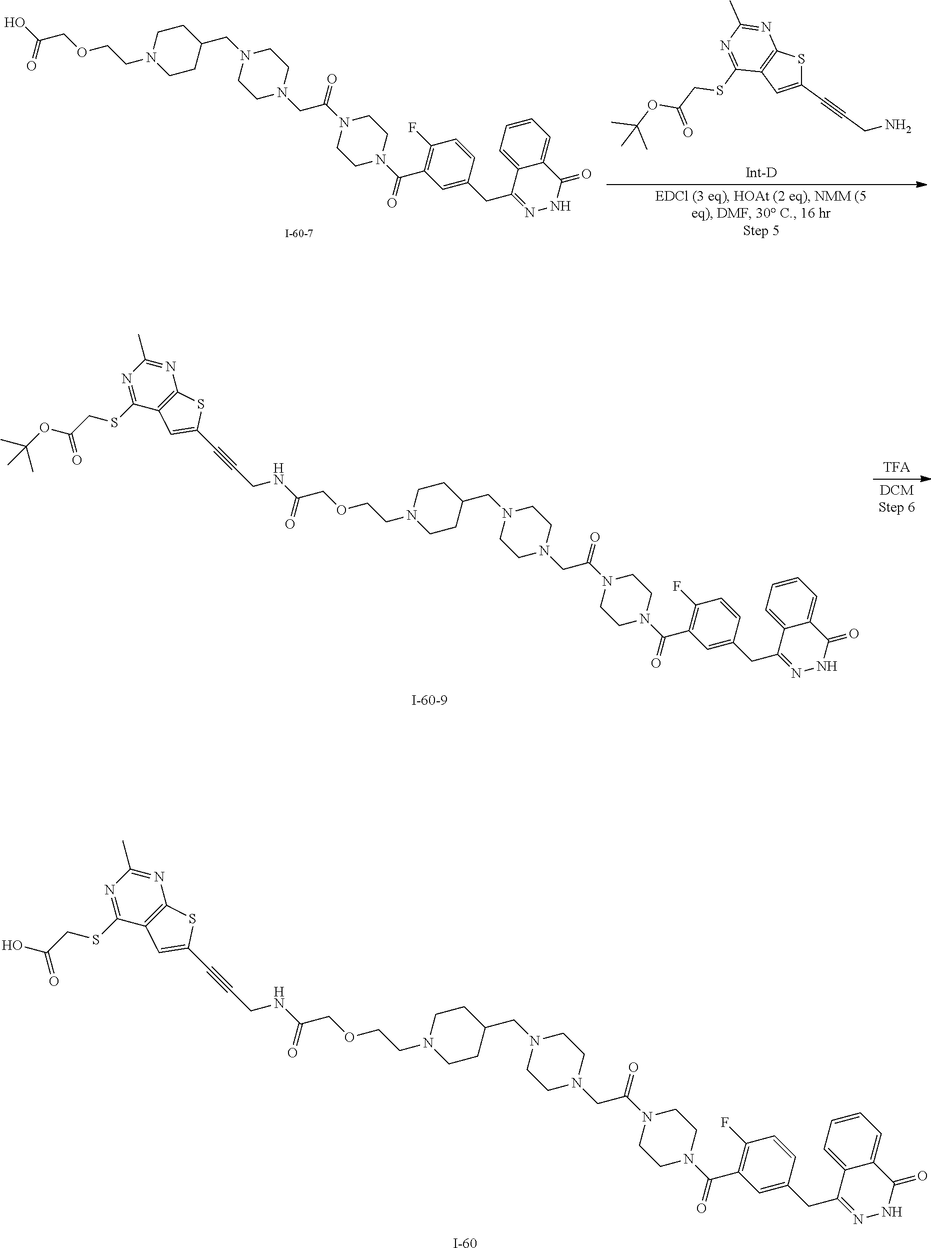

### Step 1: Synthesis of I-30-3

To a solution of Int-AA (500 mg, 1.13 mmol, 1 eq) in DMF (5 mL) was added 14-1 (639.94 mg, 2.26 mmol, 2 eq) and DIPEA (291.83 mg, 2.26 mmol, 393.30 μL, 2 eq). The mixture was stirred at 30° C. for 2 hrs. The reaction mixture was washed with water (80 mL) and extracted with EA (30 mL*3). The combined organic phase was washed with brine (50 mL*2), dried with anhydrous Na₂SO₄, filtered and concentrated under vacuum to give I-30-3 (850 mg, 1.11 mmol, 98.23% yield, 90% purity) as a brown gum. I-60-3 was used to next step without further purification. LCMS (Method E): Rt=0.422 min, [M+H]⁺=690.5.

### Step 2: Synthesis of I-60-4

I-30-3 (850 mg, 1.23 mmol, 1 eq) was dissolved in HCl/MeOH (4 M, 8.50 mL, 27.59 eq). The mixture was stirred at 25° C. for 1 hr. The reaction mixture was concentrated under reduced pressure to give I-60-4 (800 mg, 1.02 mmol, 82.95% yield, 80% purity, HCl) as a brown foam and was used to next step without further purification. LCMS (Method E): Rt=0.347 min, [M+H]⁺=590.4.

### Step 3: Synthesis of I-60-6

To a solution of I-60-4 (500 mg, 847.89 μmol, 1 eq) in CH₃CN (5 mL) was added I-60-5 (202.73 mg, 847.89 μmol, 1 eq) and Cs₂CO₃ (828.77 mg, 2.54 mmol, 3 eq). The mixture was stirred at 80° C. for 15 hrs. The reaction mixture was diluted with 1 mL CH₃CN and filtered. The filtrate was used for purification. The crude product was purified by reversed-phase HPLC (0.1% NH₃·H₂O). I-60-6 (280 mg, 355.66 mol, 41.95% yield, 95% purity) was obtained as a brown gum. LCM (Method G)S: Rt=0.620 min, [M+H]⁺=748.5.

### Step 4: Synthesis of I-60-7

To a solution of I-60-6 (280 mg, 374.38 μmol, 1 eq) in DCM (3 mL) was added TFA (3.07 g, 26.92 mmol, 2 mL, 71.92 eq). The mixture was stirred at 30° C. for 1.5 hrs. The reaction mixture was concentrated under reduced pressure to give I-60-7 (320 mg, 317.69 μmol, 84.86% yield, 80% purity, TFA) as a brown gum, which was used in the next step without further purification. LCMS (Method G): Rt=0.378 min, [M+H]⁺=692.4.

### Step 5: Synthesis of I-60-9

To a solution of I-60-7 (300 mg, 433.66 μmol, 1 eq) and Int-D (227.33 mg, 650.49 μmol, 1.5 eq) in DMF (5 mL) was added EDCI (249.40 mg, 1.30 mmol, 3 eq), HOAt (118.05 mg, 867.31 μmol, 121.33 L, 2 eq) and NMM (219.32 mg, 2.17 mmol, 238.39 μL, 5 eq). The mixture was stirred at 30° C. for 16 hrs. The reaction mixture was diluted with DMF (1 mL) and filtered. The filtrate was purified by reversed-phase HPLC (0.1% NH₃·H₂O). I-60-9 (30 mg, 20.52 μmol, 4.73% yield, 70% purity) was obtained as a yellow gum. LCMS (Method E): Rt=0.463 min, [M+H]⁺=1023.7.

### Step 6: Synthesis of I-60

To a solution of I-60-9 (30 mg, 29.32 μmol, 1 eq) in DCM (0.3 mL) was added TFA (153.50 mg, 1.35 mmol, 0.1 mL, 45.92 eq). The mixture was stirred at 30° C. for 2 hrs. The mixture was filtered and the filtrate was concentrated. The residue was purified by prep-HPLC (column: Waters Xbridge 150 mm*25 mm*5 um; mobile phase: [water (ammonia hydroxide v/v):ACN]; gradient: 0%-30% ACN over min). I-60 (4.53 mg, 4.66 μmol, 15.89% yield, 99.484% purity) was obtained as an off-white solid. LCMS (Method E): Rt=0.408 min, [M+H]⁺=967.6. ¹H NMR (400 MHz, methanol-d₄) δ 8.37 (m, 1H), 7.99-7.92 (m, 1H), 7.90-7.80 (m, 2H), 7.55-7.49 (m, 1H), 7.47 (m, 1H), 7.18 (m, 1H), 4.39 (s, 2H), 4.35 (s, 2H), 4.16 (m, 4H), 4.03-3.97 (m, 1H), 3.91 (m, 3H), 3.84-3.80 (m, 1H), 3.80-3.72 (m, 2H), 3.70 (m, 2H), 3.67-3.49 (m, 4H), 3.48-3.35 (m, 5H), 3.18 (br d, J=2.4 Hz, 3H), 3.11-2.90 (m, 6H), 2.67 (s, 5H), 2.16-1.92 (m, 4H), 1.68-1.57 (m, 2H), 1.34-1.16 (m, 4H), 0.95-0.83 (m, 2H).

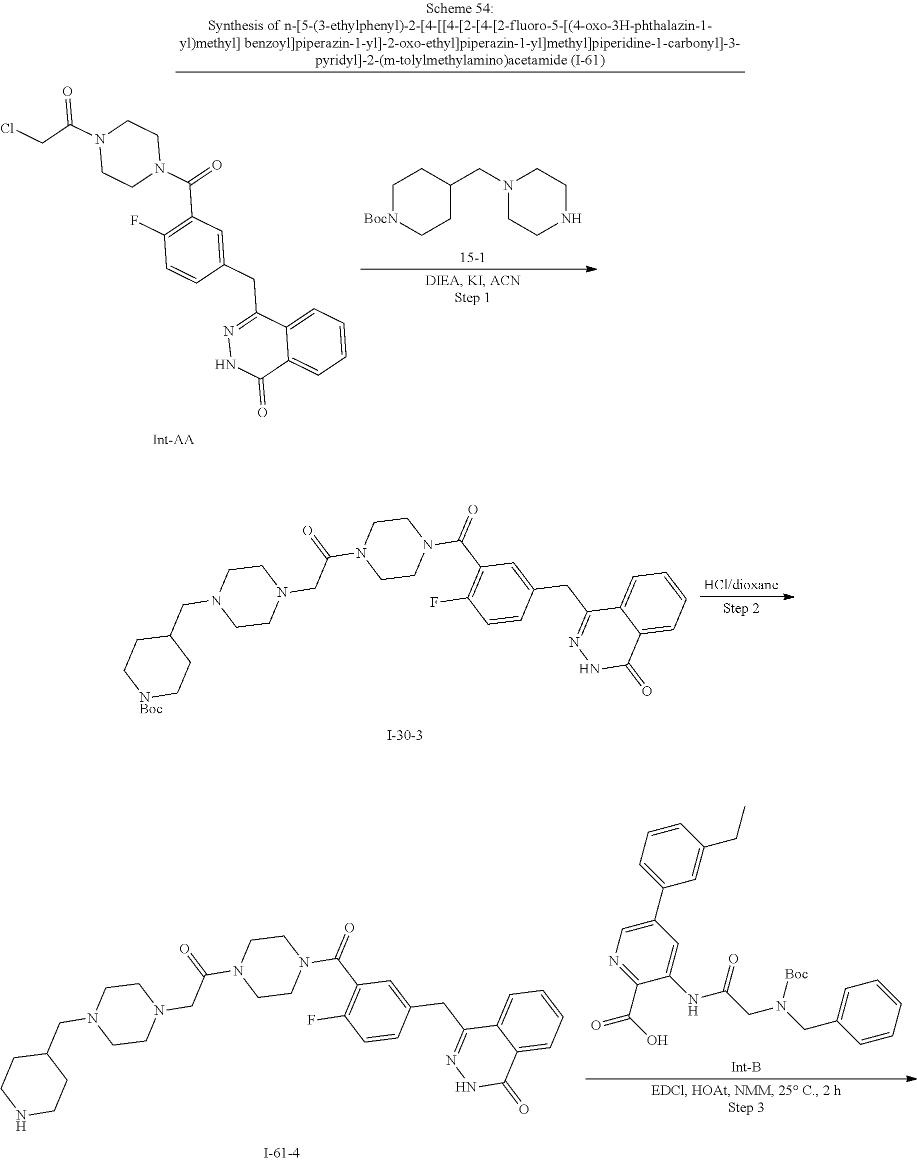

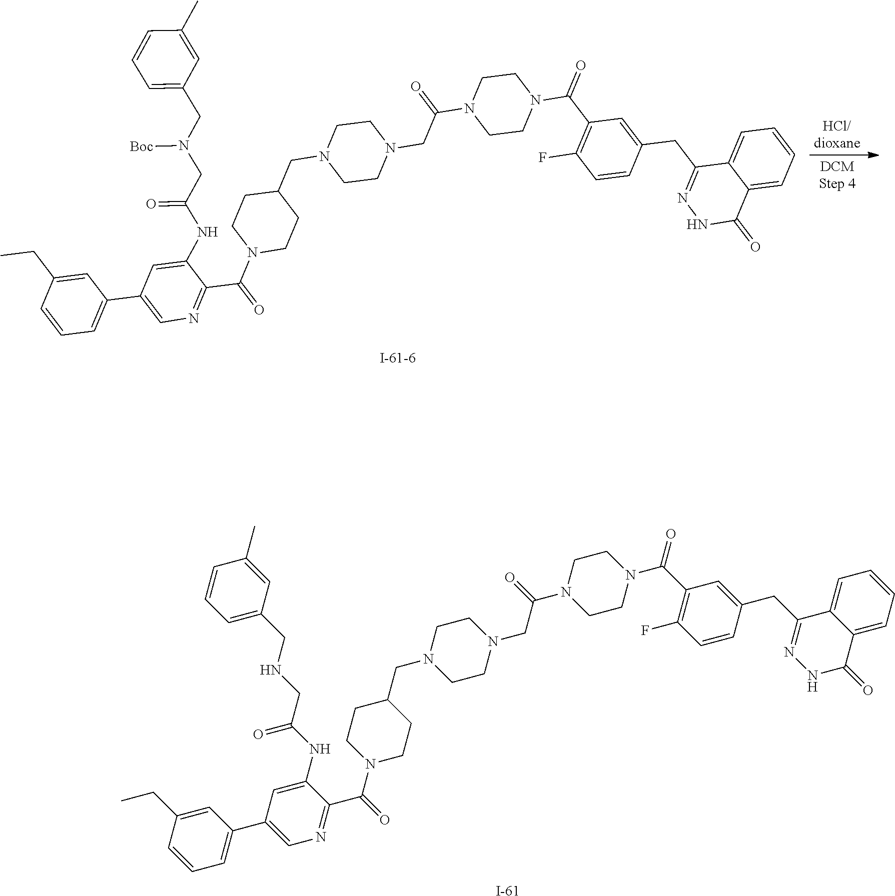

### Step 1: Synthesis of I-30-3

To a solution of Int-AA (100 mg, 225.80 μmol, 1 eq) and 14-1 (83.19 mg, 293.54 μmol, 1.3 eq) in ACN (3 mL) was added KI (112.45 mg, 677.40 μmol, 3 eq) and DIEA (116.73 mg, 903.20 μmol, 157.32 L, 4 eq). The mixture was stirred at 40° C. for 2 hr. The mixture was diluted with water (5 mL) and extracted with DCM (5 mL*3). The combined organic layer was dried over Na₂SO₄, filtered and concentrated under vacuum to give a residue. The residue was purified by column chromatography (SiO₂, PE:EA=5:1 to DCM:MeOH=0:1), then the organic liquid was concentrated to give I-30-3 (150 mg, 180.48 μmol, 79.93% yield, 83% purity) as a white solid. LCMS (Method D): Rt=0.298 min, [M+H]⁺=690.3.

### Step 2: Synthesis of I-61-4

To a solution of I-30-3 (130 mg, 188.46 μmol, 1 eq) in DCM (0.2 mL) was added HCl/dioxane (4 M, 1.30 mL, 27.59 eq). The mixture was stirred at 25° C. for 0.2 hr. The reaction mixture was concentrated under vacuum to give a residue. The residue was used in the next step directly without further purification. I-61-4 (90 mg, crude, HCl salt) was obtained as a white solid. LCMS (Method A): Rt=0.280 min, [M+H]⁺=590.4.

### Step 3: Synthesis of I-61-6

To a solution of I-61-4 (30 mg, 59.57 μmol, 1 eq) and Int-B (55.95 mg, 89.36 μmol, 1.5 eq, HCl) in DMF (1 mL) was added EDCI (34.26 mg, 178.72 μmol, 3 eq), HOAt (8.11 mg, 59.57 μmol, 8.33 μL, 1 eq), and NMM (30.13 mg, 297.86 μmol, 32.75 μL, 5 eq). The mixture was stirred at 25° C. for 1 hr. The mixture was diluted with water (5 mL) and extracted with DCM (5 mL*3). The combined organic layer was dried over Na₂SO₄, filtered and concentrated under vacuum to give a residue. The residue was purified by prep-HPLC (column: Phenomenex luna C18 150 mm*25 mm*10 um; mobile phase: [water (FA):ACN]; gradient: 33%-63% ACN over 10 min). The eluent was concentrated under vacuum and lyophilized to give I-61-6 (30 mg, 27.90 μmol, 46.83% yield) as a white solid. LCMS (Method D): Rt=0.444 min, [M+H]⁺=1076.2

### Step 4: Synthesis of I-61

To a solution of I-61-6 (30 mg, 27.90 μmol, 1 eq) in DCM (0.3 mL) was added HCl/dioxane (4 M, 0.3 mL, 43.01 eq). The mixture was stirred at 25° C. for 0.5 hr. The reaction mixture was concentrated under vacuum to give a residue. The residue was lyophilized to give I-61 (16.13 mg, 15.94 μmol, 57.15% yield, 99% purity, HCl salt) as a white solid. LCMS (Method D): Rt=O. 350 min, [M+2H]²⁺=488.4. LCMS (Method D): Rt=0. 373 min, [M+H]⁺=975.6. ¹H NMR (400 MHz, DMSO-d₆) δ 12.60 (s, 1H), 10.51 (s, 1H), 9.70-9.45 (m, 2H), 8.73 (d, J=1.2 Hz, 1H), 8.31-8.16 (m, 2H), 8.00-7.80 (m, 3H), 7.59-7.51 (m, 2H), 7.50-7.43 (m, 2H), 7.39-7.31 (m, 5H), 7.28-7.22 (m, 2H), 4.54-4.43 (m, 1H), 4.34 (s, 2H), 4.18 (s, 2H), 3.91 (s, 2H), 3.74-3.66 (m, 2H), 3.62 (d, J=3.2 Hz, 2H), 3.57 (s, 3H), 3.39 (s, 2H), 3.25 (d, J=2.0 Hz, 2H), 3.19 (d, J=1.2 Hz, 2H), 3.14-3.01 (m, 3H), 2.88-2.79 (m, 1H), 2.70-2.67 (m, 2H), 2.34 (s, 3H), 2.18-1.76 (m, 4H), 1.24-1.21 (m, 7H). ¹⁹F NMR (376 MHz, DMSO-d₆) δ −119.72.

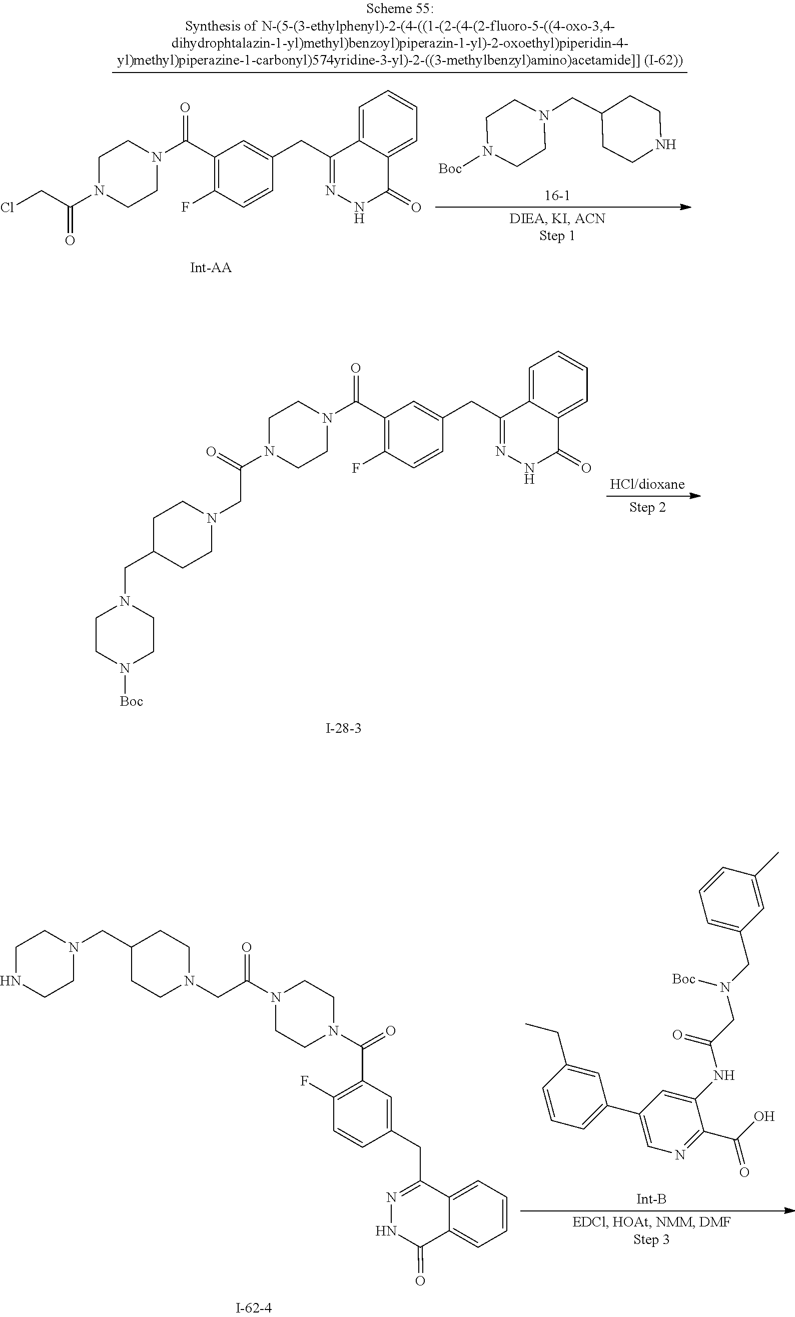 [SMILES: O=C(CCl)N1CCN(C(=O)c2cc(Cc3n[nH]c(=O)c4ccccc34)ccc2F)CC1 ; CC(C)(C)OC(=O)N1CCN(CC2CCNCC2)CC1 ; CC(C)(C)OC(=O)N1CCN(CC2CCN(CC(=O)N3CCN(C(=O)c4cc(Cc5n[nH]c(=O)c6ccccc56)ccc4F)CC3)CC2)CC1 ; O=C(CN1CCC(CN2CCNCC2)CC1)N1CCN(C(=O)c2cc(Cc3n[nH]c(=O)c4ccccc34)ccc2F)CC1 ; CCc1cccc(-c2cnc(C(=O)O)c(NC(=O)CN(Cc3cccc(C)c3)C(=O)OC(C)(C)C)c2)c1]

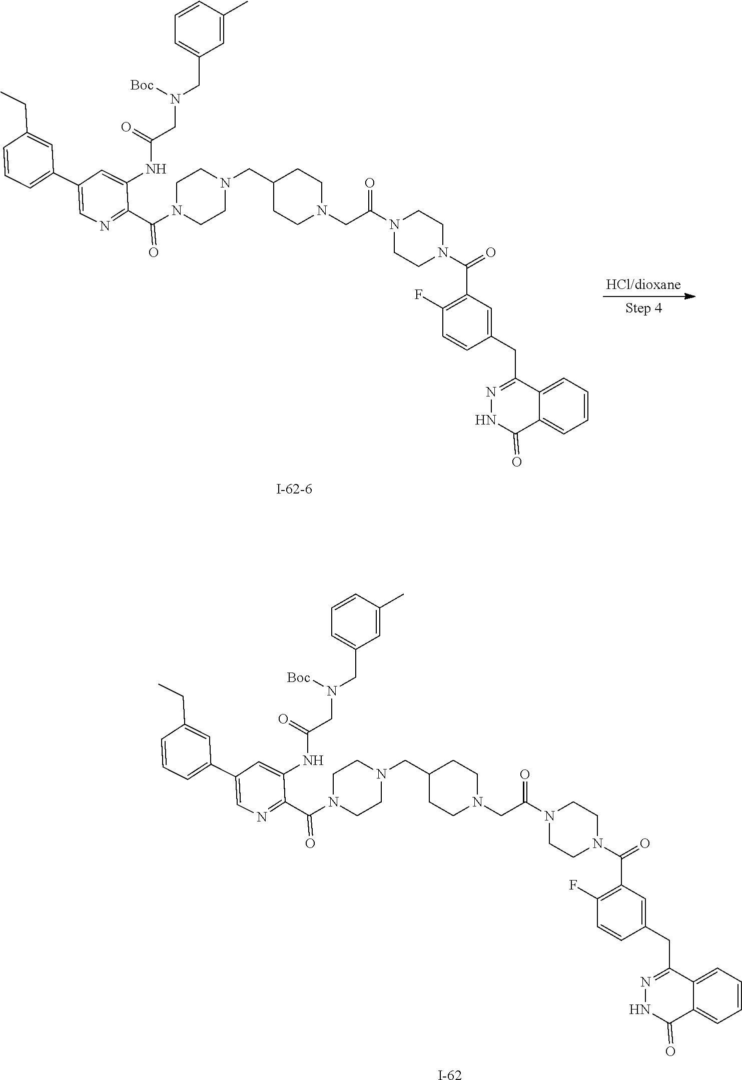

### Step 1: Synthesis of I-28-3

To a solution of 16-1 (307.17 mg, 1.08 mmol, 1.2 eq), Int-AA (307.17 mg, 1.08 mmol, 1.2 eq) in can (4 mL) was added DIEA (466.93 mg, 3.61 mmol, 629.28 μL, 4 eq) and KI (599.73 mg, 3.61 mmol, 4 eq). The mixture was stirred at 40° C. for 1 hr. The reaction mixture was added to H₂O (8 mL) and then extracted with EA (8 mL*3), the organic phase was dried with Na₂SO₄, filtered and concentrated. The residue was purified by flash silica gel chromatography (40 g SepaFlash silica flash column; eluent: 0-100% ethyl acetate/petroleum ether gradient at 60 mL/min) and the eluent was concentrated to give I-28-3 (650 mg, crude) as a black brown solid. LCMS (Method D): Rt=0.265 min, [M+H]⁺=690.4.

### Step 2: Synthesis of I-62-4

To a solution of I-28-3 (100 mg, 144.97 μmol, 1 eq) in dioxane (0.3 mL) was added HCl/dioxane (4 M, 0.6 mL, 16.56 eq). The mixture was stirred at 25° C. for 1 hr. The reaction mixture was concentrated under reduced pressure to give I-62-4 (100 mg, crude, HCl) as a brown solid. LCMS (Method D): Rt=0.223 min, [M+H]⁺=590.3.

### Step 3: Synthesis of I-62-6

To a solution of I-62-4 (100 mg, 159.70 μmol, 1 eq, HCl salt) and Int-B (80.42 mg, 159.70 μmol, 1 eq) in DMF (1 mL) was added EDCI (61.23 mg, 319.41 μmol, 2 eq), HOAt (21.74 mg, 159.70 μmol, 22.34 μL, 1 eq) and NMM (80.77 mg, 798.51 μmol, 87.79 μL, 5 eq). The mixture was stirred at 60° C. for 1 hr. The reaction mixture was added to H₂O (5 mL), extracted with DCM (10 mL*3), and the combined organic phase was dried over Na₂SO₄, filtered and concentrated. The residue was purified by flash silica gel chromatography (12 g SepaFlash silica flash column; eluent: 0-100% methanol/dichloromethane ether gradient at 30 mL/min) and the eluent was concentrated to give I-62-6 (150 mg, 112.99 μmol, 70.75% yield, 81% purity) as a white solid. LCMS (Method D): Rt=0.397 min, [M+2H-Boc]²+=488.6 LCMS (Method D): Rt=0.438 min, [M+2H-Boc]²⁺=488.3.

### Step 4: Synthesis of I-62

To a solution of I-62-6 (150 mg, 112.99 μmol, 1 eq) in dioxane (0.5 mL) was added HCl/dioxane (4 M, 1 mL, 35.40 eq). The mixture was stirred at 25° C. for 1 hr. The reaction mixture was concentrated under reduced pressure to give a residue. The residue was purified by prep-HPLC (column: Phenomenex luna C18 150 mm*25 mm*10 um; mobile phase: [water (FA):ACN]; gradient: 13%-43% ACN) the eluent was concentrated to remove ACN and lyophilized to yield I-62 (10 mg, 9.79 μmol, 8.66% yield, 99% purity, FA salt) as a white solid. LCMS (Method D): Rt=0.332 min, [M+H]⁺=975.5. ¹H NMR (400 MHz, methanol-d₄) δ 8.63 (d, J=4.4 Hz, 2H), 8.43-8.33 (m, 2H), 7.99-7.92 (m, 1H), 7.91-7.80 (m, 2H), 7.55-7.47 (m, 3H), 7.43 (t, J=7.6 Hz, 1H), 7.40-7.34 (m, 1H), 7.34-7.28 (m, 2H), 7.24 (d, J=4.4 Hz, 2H), 7.17 (t, J=8.8 Hz, 1H), 7.11 (d, J=4.0 Hz, 1H), 4.39 (s, 2H), 3.91 (s, 1H), 3.88 (s, 2H), 3.84-3.74 (m, 5H), 3.71 (d, J=3.6 Hz, 1H), 3.59 (s, 1H), 3.52 (s, 5H), 3.42 (d, J=3.2 Hz, 1H), 3.39-3.32 (m, 4H), 2.79-2.65 (m, 4H), 2.50 (s, 4H), 2.35 (s, 3H), 2.28 (t, J=5.6 Hz, 2H), 2.00-1.90 (m, 2H), 1.84-1.72 (m, 1H), 1.52-1.37 (m, 2H), 1.29 (t, J=7.6 Hz, 3H). ¹⁹F NMR (376 MHz, methanol-d₄) δ −120.689

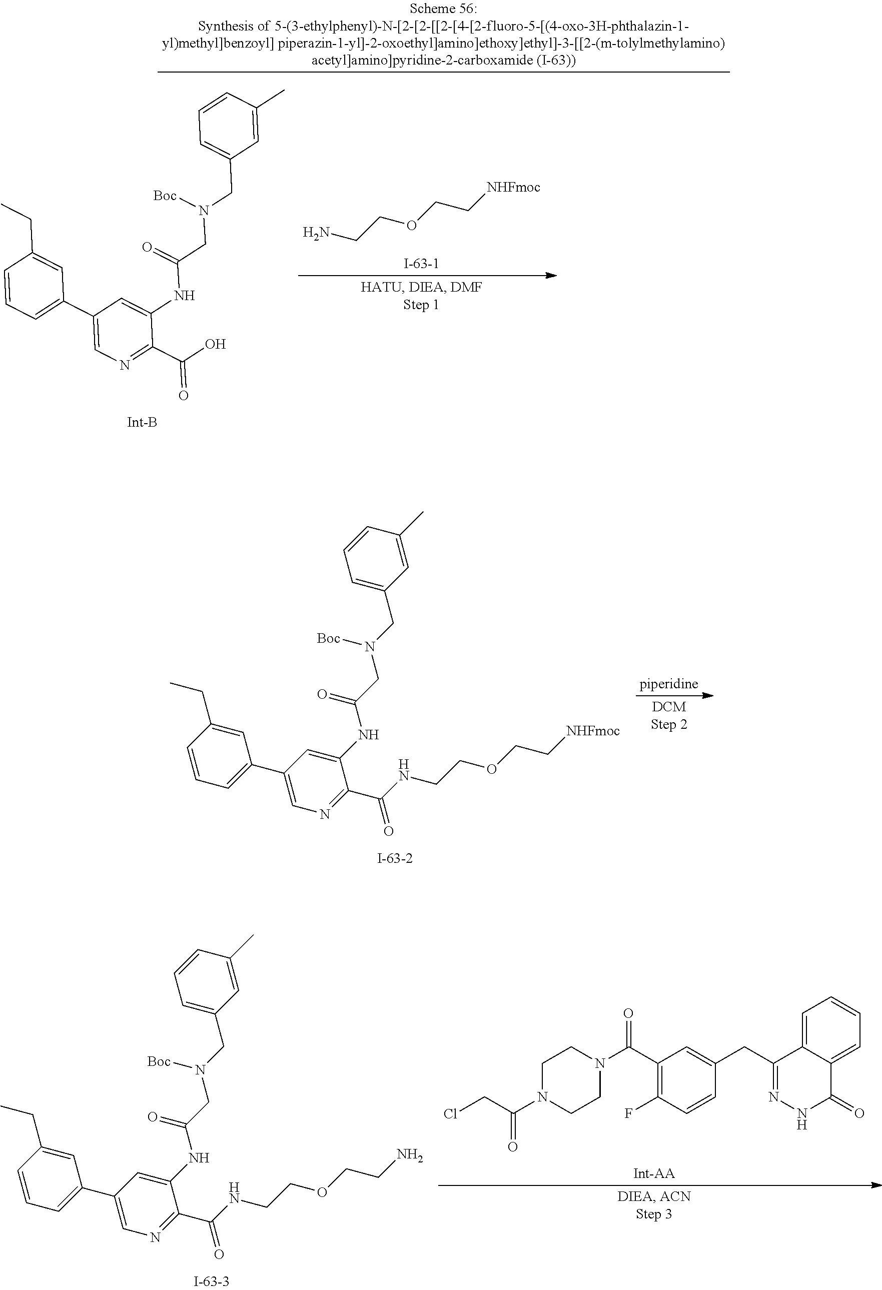

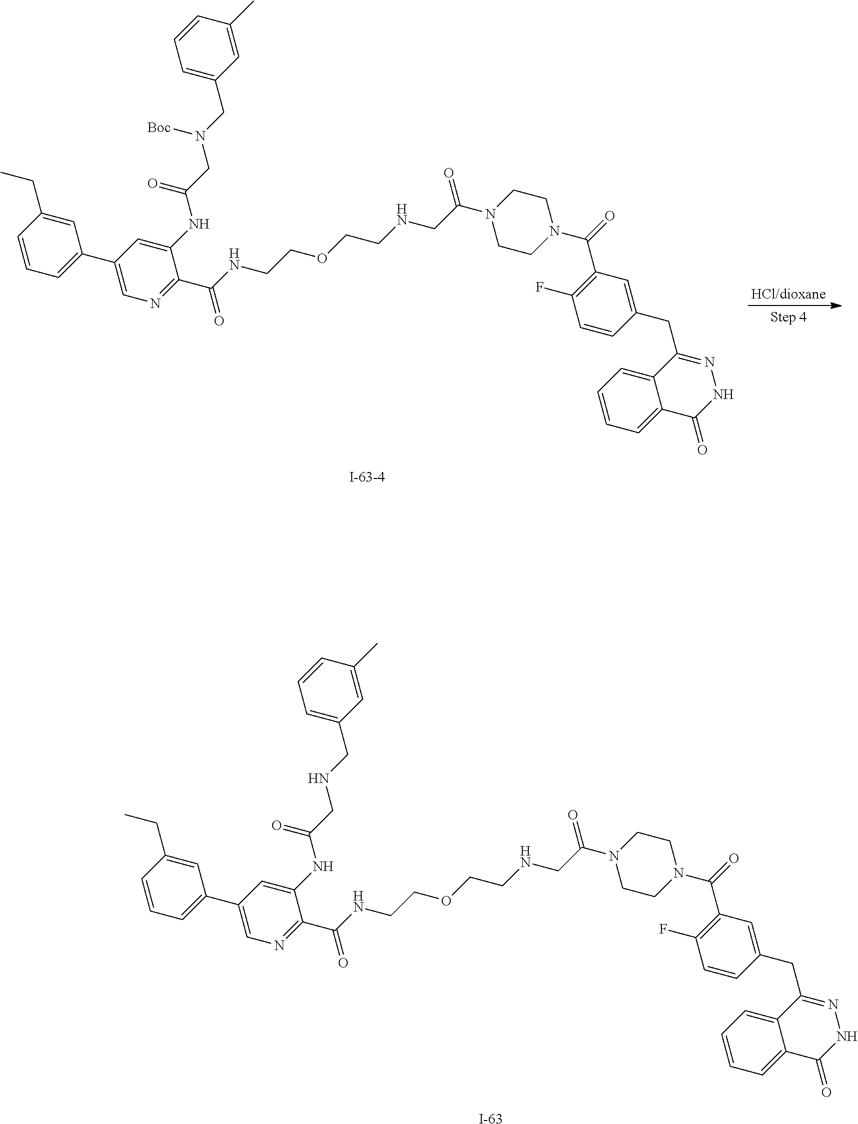

### Step 1: Synthesis of I-63-2

To a solution of Int-B (150 mg, 297.86 μmol, 1 eq) and I-63-1 (194.44 mg, 595.72 μmol, 2 eq) in DMF (1 mL) was added HATU (226.51 mg, 595.72 μmol, 2 eq) and DIEA (76.99 mg, 595.72 μmol, 103.76 L, 2 eq). The mixture was stirred at 25° C. for 1 hr. The mixture was diluted with water (5 mL) and extracted with DCM (5 mL*3). The combined organic layer was dried over Na₂SO₄, filtered and concentrated under vacuum to give a residue. The residue was purified by column chromatography (SiO₂, PE:EA=5:1 to DCM:MeOH=0:1), then the eluent was concentrated under vacuum to give I-63-2 (150 mg, 179.20 μmol, 60.16% yield, 97% purity) as a light yellow oil. LCMS (Method D): Rt=0.582 min, [M+H]⁺=812.4.

### Step 2: Synthesis of I-63-3

To a solution of I-63-2 (150 mg, 184.74 μmol, 1 eq) in DCM (1 mL) was added piperidine (47.19 mg, 554.21 μmol, 54.73 μL, 3 eq). The mixture was stirred at 25° C. for 1 hr. The mixture was purified directly without further work up. The residue was purified by column chromatography (SiO₂, PE:EA=5:1 to DCM:MeOH=0:1). The eluent was concentrated under vacuum to give I-63-3 (80 mg, 134.30 μmol, 72.70% yield, 99% purity) as a light yellow solid. LCMS (Method A): Rt=0.465 min, [M+H]⁺=590.6.

### Step 3: Synthesis of I-63-4

To a solution of I-63-3 (40 mg, 67.83 μmol, 1 eq) and Int-AA (33.04 mg, 74.61 μmol, 1.1 eq) in can (1 mL) was added DIEA (35.07 mg, 271.31 μmol, 47.26 μL, 4 eq). The mixture was stirred at 60° C. for 2 hr. The mixture was diluted with water (5 mL) and extracted with DCM (5 mL*3). The combined organic layer was dried over Na₂SO₄, filtered and concentrated under vacuum to give a residue. The residue was purified by reverse phase HPLC (H_{can}ACN), then the eluent was concentrated under vacuum and lyophilized to give I-63-4 (100 mg, crude) as a white solid. LCMS: Rt=0.839 min, [M+H]⁺=996.3.

### Step 4: Synthesis of I-63

A solution of I-63-4 (0.02 g, 20.08 μmol, 1 eq) in HCl/dioxane (4 M, 0.2 mL, 39.85 eq) was stirred at 25° C. for 0.5 hr. The mixture was concentrated, dissolved in water (1 mL) and ACN (0.5 mL), and lyophilized to give I-63 (14 mg, 14.43 μmol, 71.89% yield, 96.14% purity, HCl salt) as a white solid. LCMS (Method D): Rt=0.375 min, [M+H]⁺=896.6. ¹H NMR (400 MHz, methanol-d₄) δ 9.23 (s, 1H), 8.64 (d, J=11.6 Hz, 1H), 8.35 (d, J=7.2 Hz, 1H), 8.00-7.91 (m, 1H), 7.87-7.83 (m, 2H), 7.53-7.46 (m, 3H), 7.45-7.40 (m, 1H), 7.39-7.28 (m, 6H), 7.14 (t, J=8.8 Hz, 1H), 4.37-4.29 (m, 4H), 4.22-4.11 (m, 4H), 3.86 (s, 2H), 3.78-3.59 (m, 8H), 3.45 (d, J=4.0 Hz, 3H), 3.25 (d, J=2.8 Hz, 2H), 2.75-2.71 (m, 2H), 2.38 (d, J=7.2 Hz, 3H), 1.32-1.23 (m, 4H). ¹⁹F NMR (376 MHz, methanol-d₄) δ −120.58.

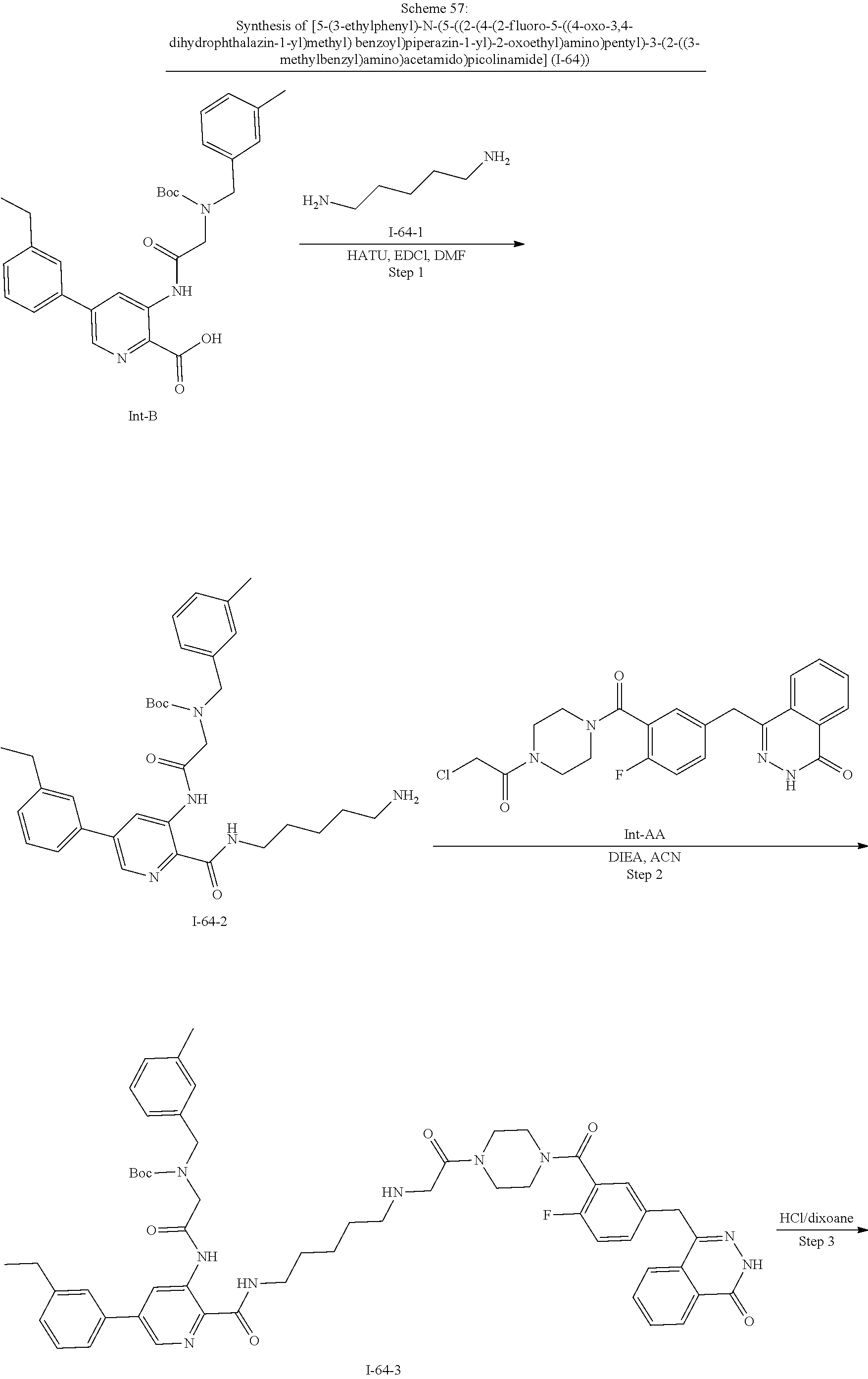 [SMILES: CCc1cccc(-c2cnc(C(=O)O)c(NC(=O)CN(Cc3cccc(C)c3)C(=O)OC(C)(C)C)c2)c1 ; NCCCCCN ; CCc1cccc(-c2cnc(C(=O)NCCCCCN)c(NC(=O)CN(Cc3cccc(C)c3)C(=O)OC(C)(C)C)c2)c1 ; O=C(CCl)N1CCN(C(=O)c2cc(Cc3n[nH]c(=O)c4ccccc34)ccc2F)CC1 ; CCc1cccc(-c2cnc(C(=O)NCCCCCNCC(=O)N3CCN(C(=O)c4cc(Cc5n[nH]c(=O)c6ccccc56)ccc4F)CC3)c(NC(=O)CN(Cc3cccc(C)c3)C(=O)OC(C)(C)C)c2)c1]

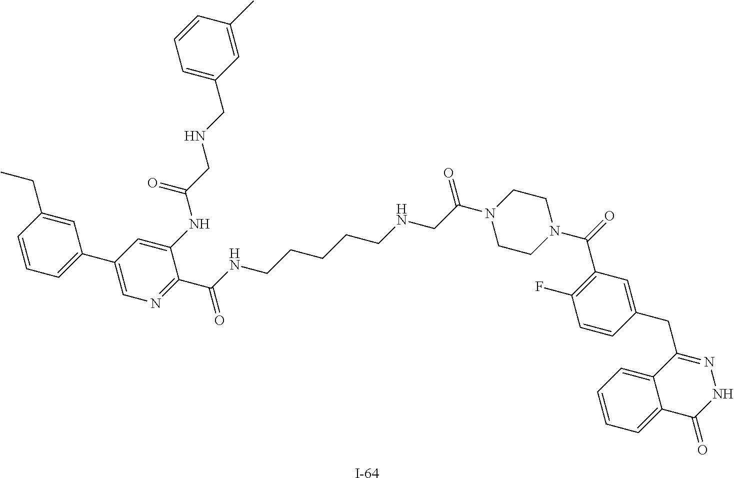

### Step 1: Synthesis of I-64-2

To solution of Int-B (250 mg, 496.44 μmol, 1 eq) in DMF (3 mL) was added HATU (226.51 mg, 595.72 μmol, 1.2 eq) and DIEA (192.48 mg, 1.49 mmol, 259.41 μL, 3 eq) and was stirred at 25° C. for 0.1 hr. Then I-64-1 (202.90 mg, 1.99 mmol, 232.42 μL, 4 eq) was added. The mixture was stirred at 25° C. for 1 hr. The reaction mixture was diluted with H₂O (5 mL) and extracted with EA (5 mL*3). The combined organic layers were washed with brine (5 mL*3), dried over Na₂SO₄, filtered and concentrated under reduced pressure to give a residue. The residue was purified by flash silica gel chromatography (20 g SepaFlash silica flash column; eluent: 0-12% methanol/dichloromethane at 80 mL/min) and the eluent was concentrated under reduced pressure to give I-64-2 (128 mg, 217.78 μmol, 43.87% yield) as a yellow oil. LCMS (Method D): Rt=0.428 min, [M+H]⁺=588.3. ¹H NMR (400 MHz, chloroform-d) δ 12.80-12.45 (m, 1H), 9.38 (s, 1H), 8.50-8.31 (m, 2H), 7.48 (s, 2H), 7.42 (d, J=7.6 Hz, 1H), 7.24-7.18 (m, 1H), 7.13-7.02 (m, 3H), 4.68-4.56 (m, 2H), 4.13-3.91 (m, 2H), 3.49-3.42 (m, 2H), 2.82-2.67 (m, 4H), 2.33 (s, 3H), 1.71-1.65 (m, 4H), 1.56 (s, 9H), 1.44 (s, 2H), 1.32-1.25 (m, 3H).

### Step 2: Synthesis of I-64-3

To a solution of 1-64-2 (120 mg, 204.17 μmol, 1 eq) m ACN (2 mL) was added Int-AA (54.25 mg, 122.50 μmol, 0.6 eq) and DIEA (79.16 mg, 612.50 μmol, 106.69 μL, 3 eq). The mixture was stirred at 25° C. for 1 hr. The reaction mixture was concentrated under reduced pressure to remove ACN. The residue was purified by prep-HPLC (FA condition) and the eluent was concentrated under reduced pressure to give I-64-3 (40 mg, 40.24 μmol, 19.71% yield) as a white solid. LCMS (Method D): Rt=0.498 min, [M+H]⁺=994.6. ¹H NMR (400 MHz, chloroform-d) δ 10.68-10.16 (m, 1H), 9.37 (s, 1H), 8.49-8.42 (m, 2H), 8.38-8.28 (m, 1H), 7.83-7.66 (m, 3H), 7.48 (s, 2H), 7.45-7.40 (m, 1H), 7.36-7.27 (m, 3H), 7.24-7.17 (m, 1H), 7.12-7.01 (m, 4H), 4.70-4.55 (m, 2H), 4.28 (s, 2H), 4.09 (s, 1H), 3.94 (s, 1H), 3.81-3.56 (m, 6H), 3.48-3.29 (m, 8H), 2.87-2.68 (m, 4H), 2.32 (s, 3H), 1.72-1.66 (m, 3H), 1.60-1.40 (m, 12H), 1.35-1.23 (m, 3H).

### Step 3: Synthesis of I-64

To a solution of I-64-3 (35 mg, 35.21 μmol, 1 eq) in DCM (0.5 mL) was added HCl/dioxane (4 M, 0.5 mL). The mixture was stirred at 25° C. for 0.5 hr. The reaction mixture was concentrated under reduced pressure to remove DCM. The crude product was dissolved in ACN (1 mL) and water (10 ml), then lyophilized to give I-64 (26.41 mg, 27.94 μmol, 79.37% yield, 98.4% purity, HCl salt) as a white solid. LCMS (Method D): Rt=0.357 min, [M+H]⁺=894.4 LCMS (Method D): Rt=0.394 min, [M+H]⁺=894.7 ¹H NMR (400 MHz, methanol-d₄) δ 9.22 (s, 1H), 8.63 (s, 1H), 8.36 (d, J=7.6 Hz, 1H), 7.94 (d, J=4.4 Hz, 1H), 7.89-7.78 (m, 2H), 7.51 (d, J=11.2 Hz, 3H), 7.48-7.42 (m, 1H), 7.40-7.32 (m, 5H), 7.29 (d, J=5.6 Hz, 1H), 7.19-7.11 (m, 1H), 4.38 (s, 2H), 4.30 (s, 2H), 4.22-4.08 (m, 4H), 3.87-3.67 (m, 3H), 3.58-3.34 (m, 7H), 3.08 (s, 2H), 2.75 (m, 2H), 2.72-2.66 (s, 3H), 1.86-1.69 (m, 4H), 1.52 (d, J=4.4 Hz, 2H), 1.35-1.24 (m, 3H). ¹⁹F NMR (376 MHz, methanol-d₄) δ −120.732.

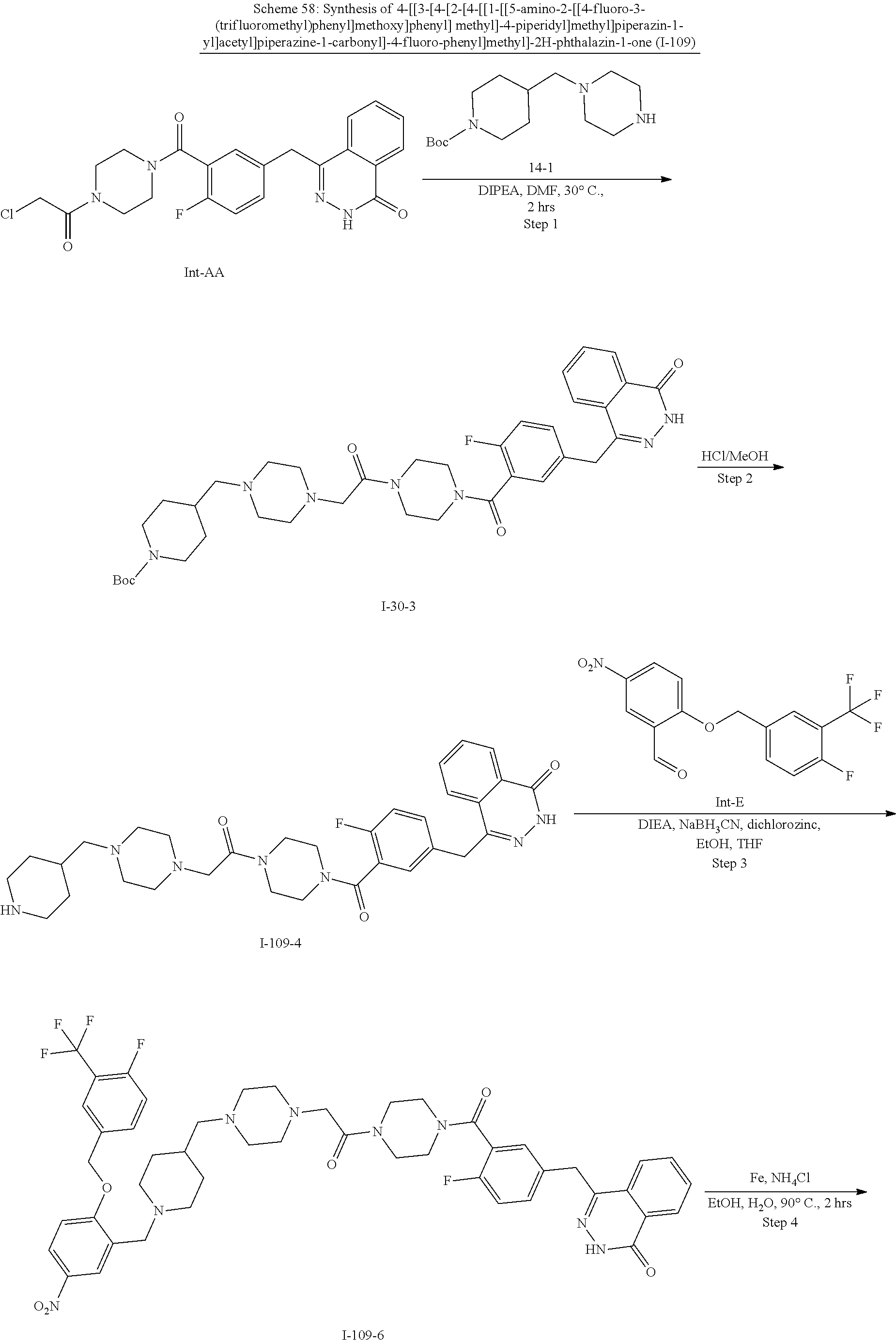

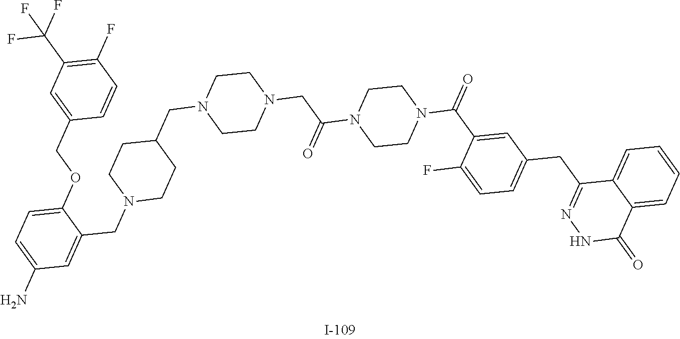 [SMILES: Nc1ccc(OCc2ccc(F)c(C(F)(F)F)c2)c(CN2CCC(CN3CCN(CC(=O)N4CCN(C(=O)c5cc(Cc6n[nH]c(=O)c7ccccc67)ccc5F)CC4)CC3)CC2)c1]

### Step 1: Synthesis of I-30-3

To a solution of Int-AA (300 mg, 677.40 μmol, 1 eq) in DMF (3 mL) was added 14-1 (383.96 mg, 1.35 mmol, 2 eq) and DIPEA (175.10 mg, 1.35 mmol, 235.98 μL, 2 eq). The mixture was stirred at 30° C. for 2 hrs. The reaction mixture was washed with water (80 mL) and extracted with EA (30 mL*3). The combined organic phase was washed with brine (50 mL*1), dried with anhydrous Na₂SO₄, filtered and concentrated under vacuum to give I-30-3 (450 mg, 554.49 μmol, 81.86% yield, 85% purity) as a yellow oil and was used directly in the next step without further purification. LCMS (Method G): Rt=0.595 min, [M+H]⁺=690.4.

### Step 2: Synthesis of I-109-4

I-30-3 (450 mg, 652.35 μmol, 1 eq) was dissolved in HCl/MeOH (4 M, 4.50 mL, 27.59 eq). The mixture was stirred at 25° C. for 1 hr. The reaction mixture was concentrated under reduced pressure to give I-109-4 (400 mg, 619.65 μmol, 94.99% yield, 97% purity, HCl) as a brown foam which was used directly in the next step. LCMS (Method E): Rt=0.427 min, [M+H]⁺=690.4.

### Step 3: Synthesis of I-109-6

To a solution of I-109-4 (100 mg, 169.58 μmol, 1 eq) and Int-E (87.31 mg, 254.37 μmol, 1.5 eq) in EtOH (1 mL) and THF (1 mL) was added DIEA (43.83 mg, 339.15 μmol, 59.07 μL, 2 eq) and dichlorozinc (2.5 M, 203.49 μL, 3 eq) and the resulting solution was stirred for 0.5 hr at 40° C., and then NaBH₃CN (31.97 mg, 508.73 μmol, 3 eq) was added. The mixture was stirred at 40° C. for 12 hrs. The residue was purified by prep-HPLC (column: Phenomenex luna C18 150 mm*25 mm*10 um; mobile phase: [water(FA):ACN]; gradient: 10%-40% ACN over 10 min). The collected mixture was lyophilized. I-109-6 (25 mg, 26.17 μmol, 15.44% yield, 96% purity) was obtained as a white solid. LCMS (Method E): Rt=0.445 min, [M+H]⁺=917.4.

### Step 4: Synthesis of I-109

To a solution of I-109-6 (25 mg, 27.26 μmol, 1 eq) in EtOH (0.5 mL) and H₂O (0.5 mL) was added Fe (9.14 mg, 163.59 μmol, 6 eq) and NH₄Cl (7.29 mg, 136.32 μmol, 5 eq). The mixture was stirred at 90° C. for 2 hrs. The resulting product was filtered. The filtrate was concentrated under vacuum. I-109 (1.91 mg, 2.00 μmol, 7.35% yield, 93% purity) was obtained as an off-white solid. LCMS (Method E): Rt=0.412 min, [M+H]⁺=887.5. ¹H NMR (400 MHz, methanol-d₄) δ 8.37 (dd, J=1.3, 7.8 Hz, 1H), 8.00-7.93 (m, 1H), 7.90-7.82 (m, 2H), 7.80 (m, 1H), 7.76-7.72 (m, 1H), 7.48 (m, 1H), 7.35 (m, 2H), 7.16 (t, J=8.9 Hz, 1H), 6.86 (d, J=8.6 Hz, 1H), 6.76 (d, J=2.6 Hz, 1H), 6.68 (dd, J=2.7, 8.6 Hz, 1H), 5.05 (s, 2H), 4.38 (s, 2H), 3.72 (m, 4H), 3.54 (m, 2H), 3.51 (m, 2H), 3.27-3.19 (m, 2H), 2.95 (m, 2H), 2.60-2.38 (m, 8H), 2.21-2.15 (m, 2H), 2.13-2.06 (m, 2H), 1.77-1.68 (m, 2H), 1.56-1.49 (m, 1H), 1.36-1.08 (m, 4H). ¹⁹F NMR: (377 MHz, methanol-d₄).

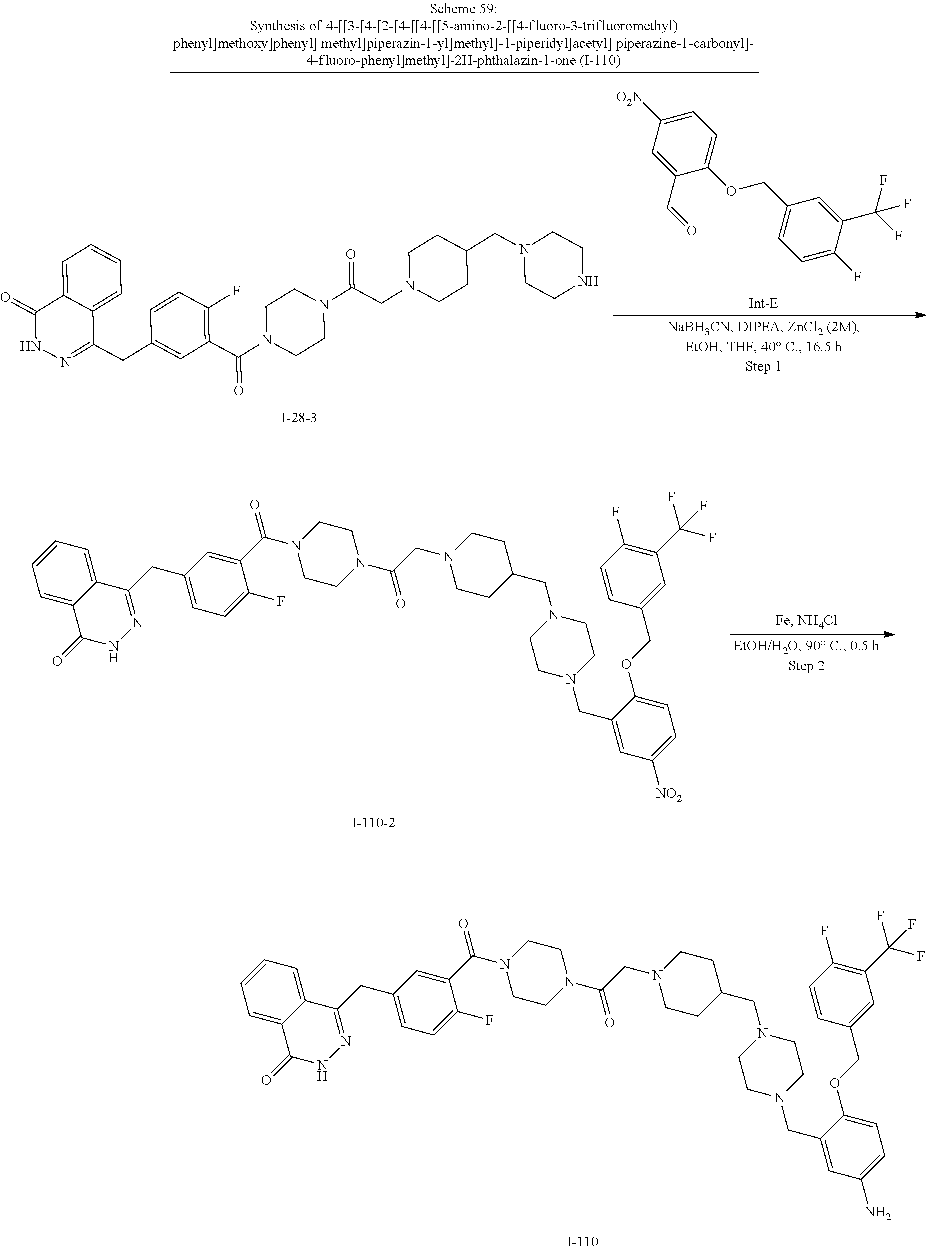 [SMILES: O=C(CN1CCC(CN2CCNCC2)CC1)N1CCN(C(=O)c2cc(Cc3n[nH]c(=O)c4ccccc34)ccc2F)CC1 ; O=Cc1cc([N+](=O)[O-])ccc1OCc1ccc(F)c(C(F)(F)F)c1 ; O=C(CN1CCC(CN2CCN(Cc3cc([N+](=O)[O-])ccc3OCc3ccc(F)c(C(F)(F)F)c3)CC2)CC1)N1CCN(C(=O)c2cc(Cc3n[nH]c(=O)c4ccccc34)ccc2F)CC1 ; Nc1ccc(OCc2ccc(F)c(C(F)(F)F)c2)c(CN2CCN(CC3CCN(CC(=O)N4CCN(C(=O)c5cc(Cc6n[nH]c(=O)c7ccccc67)ccc5F)CC4)CC3)CC2)c1]

### Step 1: Synthesis of I-110-2

To a solution of I-28-3 (70 mg, 111.79 μmol, 1 eq, HCl salt) and Int-E (95.93 mg, 279.48 μmol, 2.5 eq) in EtOH (1 mL) and THF (1 mL) was added DIPEA (28.90 mg, 223.58 μmol, 38.94 μL, 2 eq) and zinc chloride (2 M, 167.69 μL, 3 eq). The mixture was stirred at 40° C. for 0.5 h. Then NaBH₃CN (21.08 mg, 335.38 μmol, 3 eq) was added and the resulting mixture was stirred at 40° C. for 16 h. The reaction mixture was filtered and the filtrate was concentrated. The residue was purified by flash silica gel chromatography (20 g sepaflash silica flash column, eluent of 0-60% MeOH/DCM; gradient at 40 mL/min). I-110-2 (50 mg, 39.78 μmol, 35.58% yield, 72.9% purity) was obtained as a white solid. LCMS (Method G): Rt=0.754 min, [M+H]⁺=917.5.

### Step 2: Synthesis of I-110

To a solution of I-110-2 (30 mg, 32.72 μmol, 1 eq) and Fe (9.14 mg, 163.59 μmol, 5 eq) in EtOH (0.5 mL) and H₂O (0.5 mL) was added NH₄Cl (8.75 mg, 163.59 μmol, 5 eq). The mixture was stirred at 90° C. for 0.5 hr. The mixture was filtered and the filtrate was purified by prep-HPLC (column: Waters Xbridge 150 mm*25 mm*5 um; mobile phase: [water (ammonia hydroxide v/v):ACN]; gradient: 35%-65% ACN over 10 min). I-110 (4.02 mg, 4.38 μmol, 13.38% yield, 96.6% purity) was obtained as a white solid. LCMS (Method C): Rt=0.935 min, [M+H]⁺=887.5. ¹H NMR (400 MHz, CD₃OD) δ 8.42-8.32 (m, 1H), 7.99-7.93 (m, 1H), 7.91-7.81 (m, 2H), 7.80-7.70 (m, 2H), 7.48 (br dd, J=2.8, 4.2 Hz, 1H), 7.41-7.28 (m, 2H), 7.16 (t, J=9.0 Hz, 1H), 6.85 (d, J=8.5 Hz, 1H), 6.77 (d, J=2.5 Hz, 1H), 6.67 (dd, J=2.8, 8.8 Hz, 1H), 5.05 (s, 2H), 4.39 (s, 2H), 3.83-3.63 (m, 4H), 3.61-3.45 (m, 5H), 3.43-3.33 (m, 2H), 3.26-3.12 (m, 2H), 2.96-2.79 (m, 2H), 2.65-2.32 (m, 7H), 2.18 (br dd, J=6.9, 13.0 Hz, 2H), 2.10-1.96 (m, 2H), 1.80-1.63 (m, 2H), 1.59-1.42 (m, 1H), 1.34-1.10 (m, 2H).

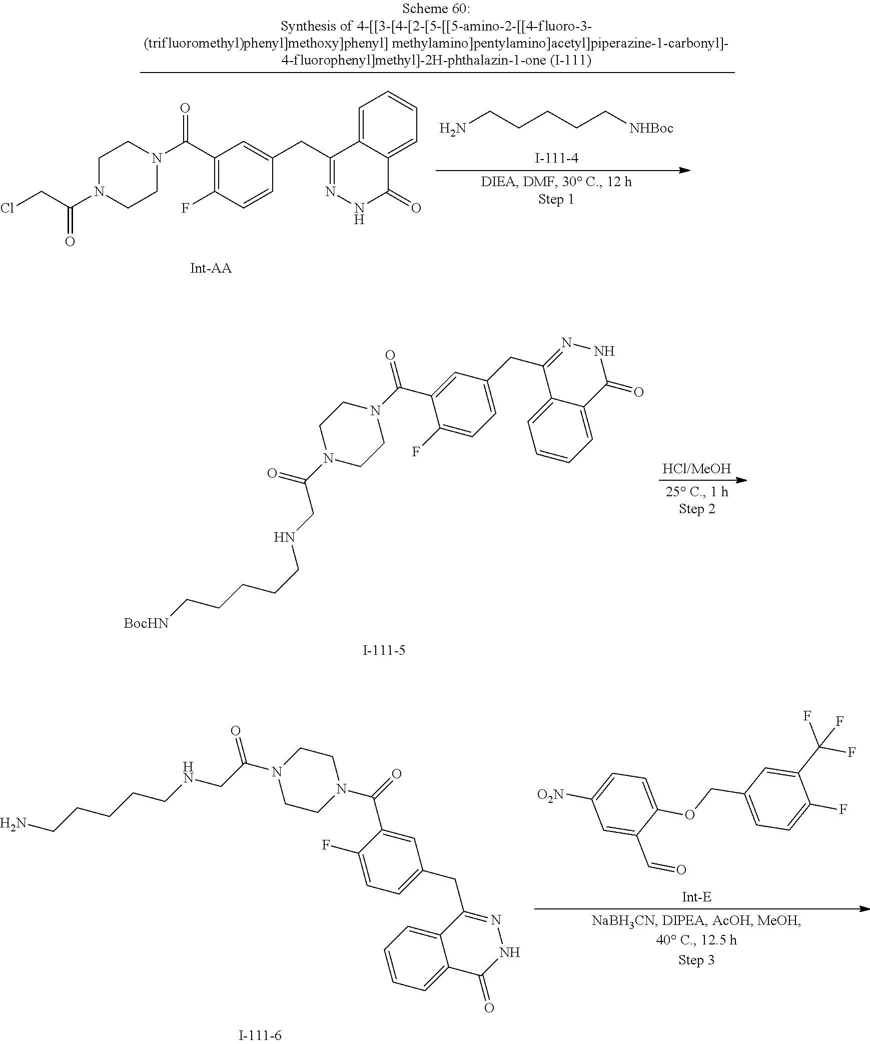

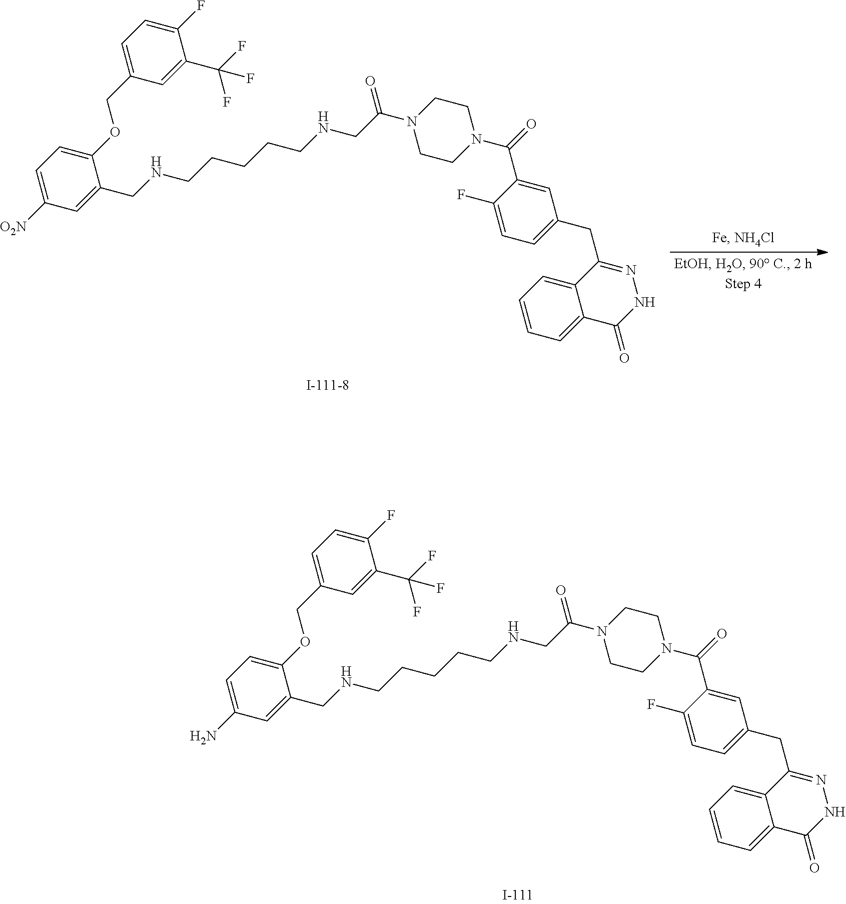 [SMILES: O=C(CNCCCCCNCc1cc([N+](=O)[O-])ccc1OCc1ccc(F)c(C(F)(F)F)c1)N1CCN(C(=O)c2cc(Cc3n[nH]c(=O)c4ccccc34)ccc2F)CC1 ; Nc1ccc(OCc2ccc(F)c(C(F)(F)F)c2)c(CNCCCCCNCC(=O)N2CCN(C(=O)c3cc(Cc4n[nH]c(=O)c5ccccc45)ccc3F)CC2)c1]

### Step 1: Synthesis of I-111-5

To a solution of Int-AA (300 mg, 677.40 umol, 1 eq) and I-111-4 (274.07 mg, 1.35 mmol, 281.96 uL, 2 eq) in DMF (3 mL) was added DIEA (175.10 mg, 1.35 mmol, 235.98 uL, 2 eq). The mixture was stirred at 30° C. for 12 hrs. The residue was diluted with H₂O (10 mL) and was extracted with ethyl acetate (10 mL*2). The combined organic phase was washed with brine (10 mL), dried with anhydrous Na₂SO₄, filtered and concentrated under vacuum. The residue was purified through flash silica gel chromatography (12 g SepaFlash silica flash column; eluent: 30-50% DCM/MeOH). I-111-5 (150 mg, 221.78 umol, 32.74% yield, 90% purity) was obtained as a white gum. LCMS (Method E): Rt=0.424 min, [M+H]⁺=609.3.

### Step 2: Synthesis of I-111-6

A solution of I-111-5 (100 mg, 164.28 umol, 1 eq) in HCl/MeOH (4 M, 1.64 mL) was stirred at 25° C. for 1 h. The mixture was concentrated under vacuum. I-111-6 (80 mg, 157.30 umol, 95.75% yield) was obtained as a yellow solid. LCMS (Method E): Rt=0.357 min, [M+H]⁺=509.1.

### Step 3: Synthesis I-111-8

To a solution of I-111-6 (80 mg, 157.30 umol, 1 eq) and Int-E (53.99 mg, 157.30 umol, 1 eq) in MeOH (1 mL) was added DIPEA (81.32 mg, 629.19 umol, 109.59 uL, 4 eq) and AcOH (37.78 mg, 629.19 umol, 36.02 uL, 4 eq). The solution was stirred for 0.5 h at 40° C., then NaBH₃CN (39.54 mg, 629.19 umol, 4 eq) was added. The mixture was stirred at 40° C. for 12 hrs. The mixture was concentrated under vacuum. The residue was purified by prep-HPLC (column: Phenomenex luna C18 150 mm*25 mm*10 um; mobile phase: [water (FA):ACN]; gradient: 10%-40% ACN over 10 min). I-111-8 (40 mg, 44.99 umol, 28.60% yield, 94% purity) was obtained as a white solid. LCMS (Method B): Rt=0.576 min, [M+H]⁺=836.4.

### Step 4: Synthesis of I-111

To a solution of I-111-8 (40 mg, 47.86 umol, 1 eq) in EtOH (0.5 mL) and H₂O (0.5 mL) was added Fe (16.04 mg, 287.14 umol, 6 eq) and NH₄Cl (12.80 mg, 239.29 umol, 5 eq). The mixture was stirred at 90° C. for 2 hrs. The resulting product was filtered and the filtrate was concentrated under vacuum. The residue was purified by prep-HPLC (column: Phenomenex Luna C18 150 mm*25 mm*10 um; mobile phase: [water (TFA):ACN]; gradient: 10%-40% ACN over 9 min). I-111 (7.15 mg, 8.87 μmol, 18.54% yield, 100% purity) was obtained as a yellow solid. LCMS (Method G): Rt=0.726 min, [M+H]⁺=806.4. ¹H NMR (400 MHz, methanol-d₄) δ 8.36 (dd, J=1.2, 7.6 Hz, 1H), 7.94 (m, 1H), 7.90-7.81 (m, 4H), 7.50 (m, 1H), 7.46-7.36 (m, 4H), 7.30-7.25 (m, 1H), 7.16 (t, J=9.2 Hz, 1H), 5.31-5.25 (m, 2H), 4.38 (s, 2H), 4.27 (s, 2H), 4.15-4.01 (m, 2H), 3.85-3.69 (m, 3H), 3.53 (m, 2H), 3.37 (m, 2H), 3.11-2.97 (m, 4H), 1.73 (m, 4H), 1.43 (m, 3H).

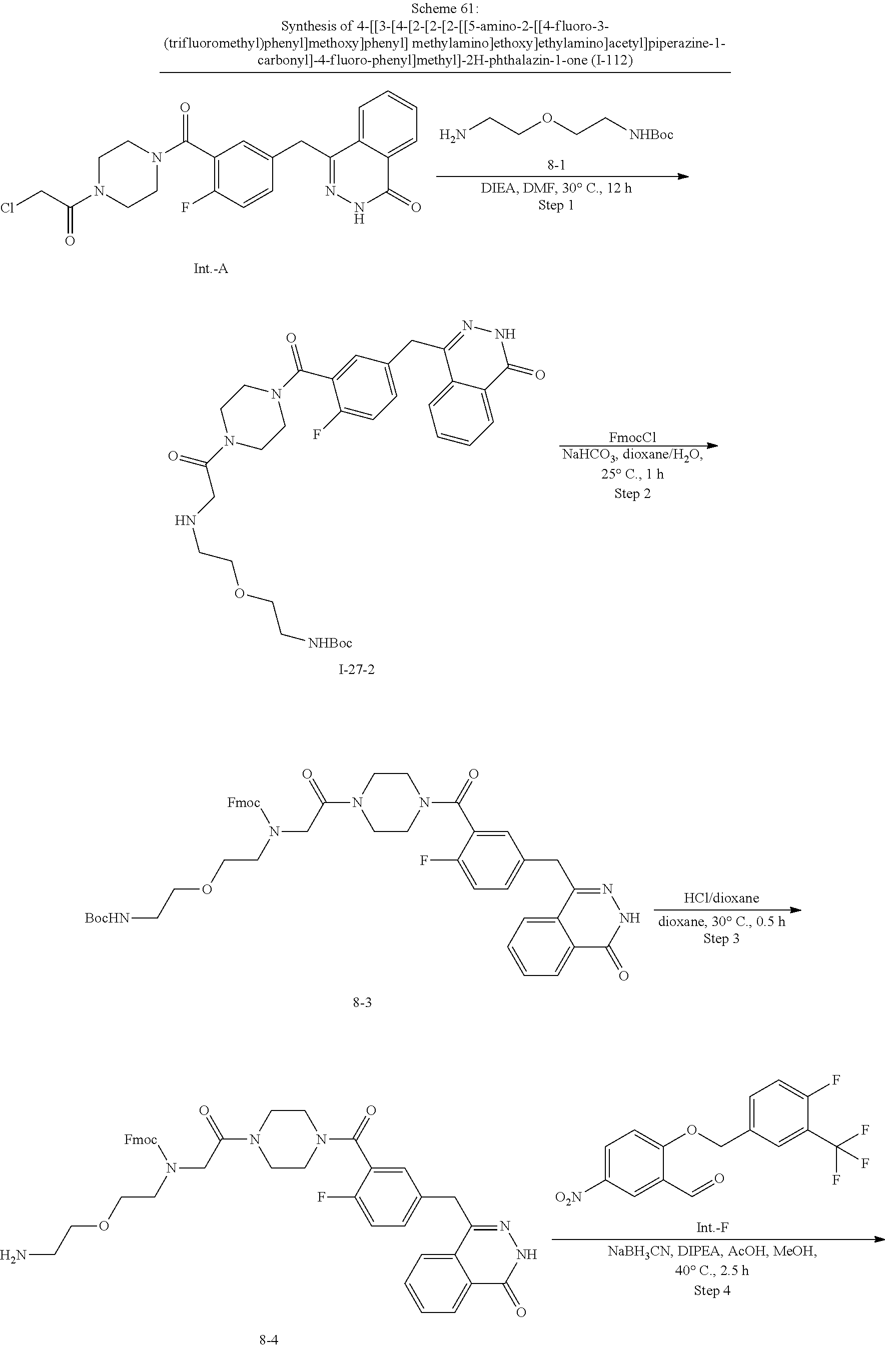

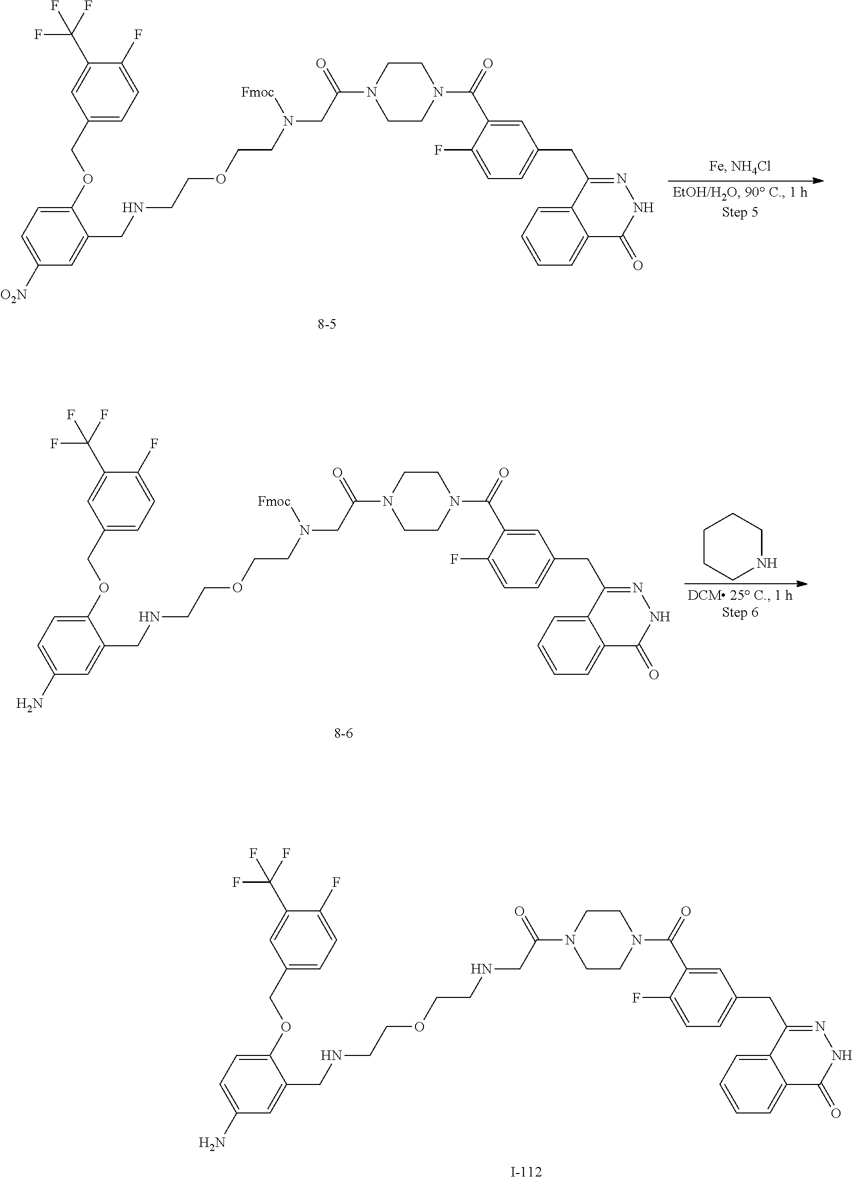

### Step 1: Synthesis of I-112-2

To a solution of Int-AA (1 g, 2.26 mmol, 1 eq) and I-112-1 (922.46 mg, 4.52 mmol, 2 eq) in DMF (10 mL) was added DIEA (583.66 mg, 4.52 mmol, 786.61 μL, 2 eq). The mixture was stirred at 30° C. for 12 hrs. The mixture was diluted with H₂O (20 mL) and extracted with ethyl acetate (25 mL*2). The combined organic layers were washed with brine (10 mL), dried with anhydrous Na₂SO₄, filtered and concentrated under vacuum. The crude was purified by flash silica gel chromatography (40 g sepaflash silica flash column; eluent of 30-50% MeOH/DCM). I-112-2 (500 mg, 769.64 μmol, 34.09% yield, 94% purity) was obtained as a yellow oil. LCMS (Method G): Rt=0.500 min, [M+H]⁺=611.4.

### Step 2: Synthesis of I-112-3

To a solution of I-112-2 (500 mg, 818.77 umol, 1 eq) and FmocCl (254.18 mg, 982.52 μmol, 1.2 eq) in dioxane (4 mL) and H₂O (2 mL) was added NaHCO₃ (103.17 mg, 1.23 mmol, 1.5 eq). The mixture was stirred at 25° C. for 1 h. The residue was diluted with H₂O (10 mL) and extracted with ethyl acetate (15 mL*2). The combined organic phases were washed with brine (10 mL), dried with anhydrous Na₂SO₄, filtered and concentrated under vacuum. The residue was purified through flash silica gel chromatography (40 g SepaFlash silica flash column; eluent: 0-10% MeOH/DCM). I-112-3 (450 mg, 486.25 μmol, 59.39% yield, 90% purity) was obtained as a yellow oil. LCMS (Method G): Rt=0.682 min, [M+H]⁺=833.5

### Step 3: Synthesis of I-112-4

A solution of I-112-3 (450 mg, 540.27 umol, 1 eq) in HCl/dioxane (4 M, 5.40 mL, 40 eq) was stirred at 30° C. for 0.5 h. The mixture was concentrated. I-112-4 (395 mg, 539.03 umol, 99.77% yield) was obtained as a yellow solid. LCMS (Method E): Rt=0.464 min, [M+H]⁺=733.4.

### Step 4: Synthesis of I-112-5

To a solution of I-112-4 (395 mg, 539.03 umol, 1 eq) and Int-E (185.01 mg, 539.03 μmol, 1 eq) in MeOH (4 mL) was added DIPEA (139.33 mg, 1.08 mmol, 187.78 μL, 2 eq) and AcOH (161.85 mg, 2.70 mmol, 154.29 μL, 5 eq). The solution was stirred for 0.5 h at 40° C., then NaBH₃CN (101.62 mg, 1.62 mmol, 3 eq) was added. The mixture was stirred at 40° C. for 2 h. The residue was diluted with H₂O (10 mL) and was extracted with ethyl acetate (15 mL*2). The combined organic phases were washed with brine (10 mL), dried with anhydrous Na₂SO₄, filtered and concentrated under vacuum. The residue was purified with flash silica gel chromatography (20 g SepaFlash silica flash column; eluent: 0-10% MeOH/DCM). I-112-5 (350 mg, 280.65 μmol, 52.07% yield, 85% purity) was obtained as a yellow oil. LCMS (Method E): Rt=0.536 min, [M+H]⁺=1060.3

### Step 5: Synthesis of I-112-6

To a solution of I-112-5 (350 mg, 330.18 umol, 1 eq) in EtOH (4 mL) and H₂O (4 mL) was added Fe (110.63 mg, 1.98 mmol, 6 eq) and NH₄Cl (88.31 mg, 1.65 mmol, 5 eq). The mixture was stirred at 90° C. for 1 h. The resulting product was filtered. The filtrate was concentrated under vacuum. I-112-6 (320 mg, 310.67 umol, 94.09% yield) was obtained as brown solid. LCMS (Method E): Rt=0.504 min, [M+H]⁺=1030.6.

### Step 6: Synthesis of I-112

To a solution of I-112-6 (300 mg, 291.25 umol, 1 eq) in DCM (3 mL) was added piperidine (247.99 mg, 2.91 mmol, 287.63 uL, 10.0 eq). The mixture was stirred at 25° C. for 1 h. The resulting product was filtered. The filtrate was concentrated under vacuum. The residue was purified by prep-HPLC (column: Phenomenex luna C18 150 mm*25 mm*10 um; mobile phase: [water (TFA):ACN]; gradient: 10%-40% ACN over 9 min). I-112 (13.26 mg, 15.92 μmol, 5.47% yield, 97% purity) was obtained as a white solid. LCMS (Method G): Rt=0.632 min, [M+H]⁺=808.5. ¹H NMR (400 MHz, methanol-d₄) δ 12.61 (s, 1H), 8.87-8.74 (m, 1H), 8.26 (m, 1H), 7.97-7.81 (m, 6H), 7.56 (m 1H), 7.51-7.45 (m, 1H), 7.40-7.32 (m, 1H), 7.29-7.24 (m, 1H), 7.11-7.02 (m, 1H), 6.97 (m, 1H), 5.18 (br s, 2H), 4.34 (s, 2H), 4.15 (m, 4H), 4.08 (br s, 1H), 3.71-3.58 (m, 8H), 3.47-3.41 (m, 2H), 3.32-3.23 (m, 2H), 3.12 (m, 4H).

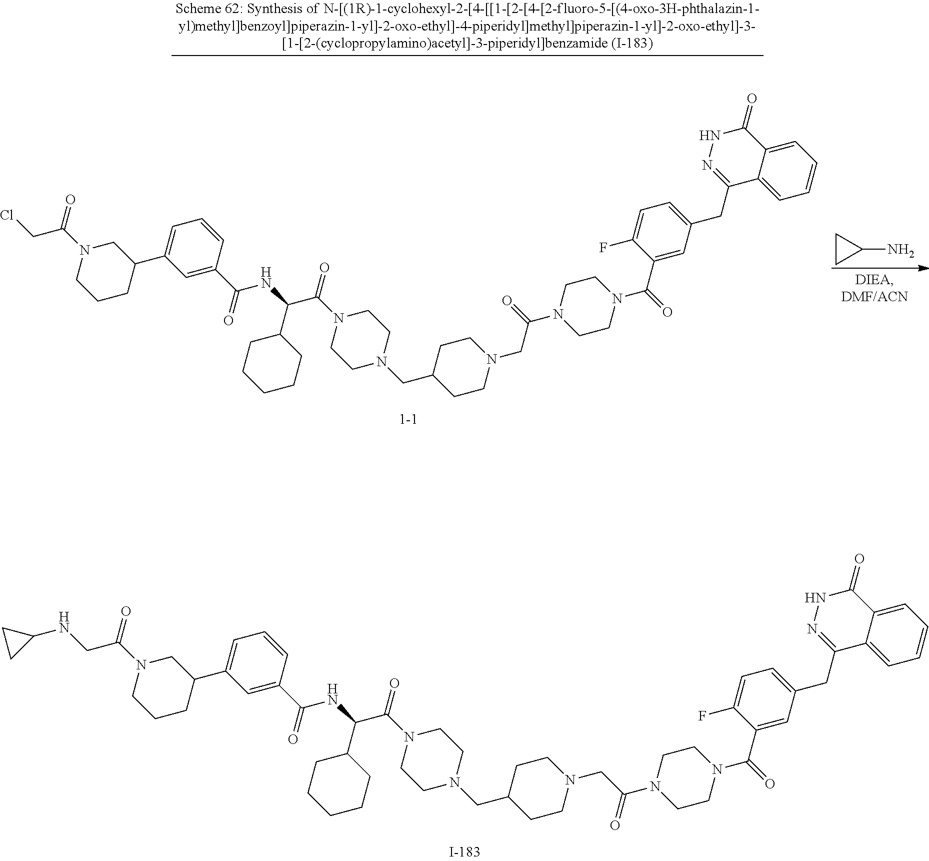

To a solution of cyclopropanamine (5.75 mg, 100.74 μmol, 6.98 μL, 2 eq) in DMF (0.5 mL) and ACN (0.5 mL) was added DIEA (26.04 mg, 201.49 μmol, 35.10 μL, 4 eq) under N₂. The reaction mixture was stirred at 25° C. for 0.5 h, and then KI (4.18 mg, 25.19 μmol, 0.5 eq) and Intermediate 1-1 (50 mg, 50.37 μmol, 1 eq) were added into the mixture. After that, the reaction mixture was stirred at 25° C. for 15.5 hrs. The reaction mixture was filtered and concentrated under the pressure to give the crude product. The crude product was purified by prep-HPLC (column: YMC-Actus Triart C18 150*30 mm*7 um; mobile phase: [water (FA)-ACN]; gradient: 8%-38% B over 10 min), and the major peak was concentrated and lyophilized to afford I-183 (21 mg, 19.82 μmol, 39.36% yield, 100% purity, FA salt). Method E: Rt=0.404 min, (M+H)+=1013.9. SFC: Rt=3.753 min, 4.347 min. ¹H NMR (400 MHz, CHLOROFORM-d) δ=10.78-10.23 (m, 1H), 8.55-8.37 (m, 1H), 7.84-7.65 (m, 5H), 7.46-7.30 (m, 4H), 7.16-7.03 (m, 2H), 5.05 (d, J=2.0 Hz, 1H), 4.70 (d, J=12.8 Hz, 1H), 4.29 (s, 2H), 3.92-2.90 (m, 21H), 2.81-2.59 (m, 2H), 2.37-2.17 (m, 5H), 2.13-1.73 (m, 10H), 1.69-1.55 (m, 3H), 1.35-1.05 (m, 8H), 0.72-0.40 (m, 4H).

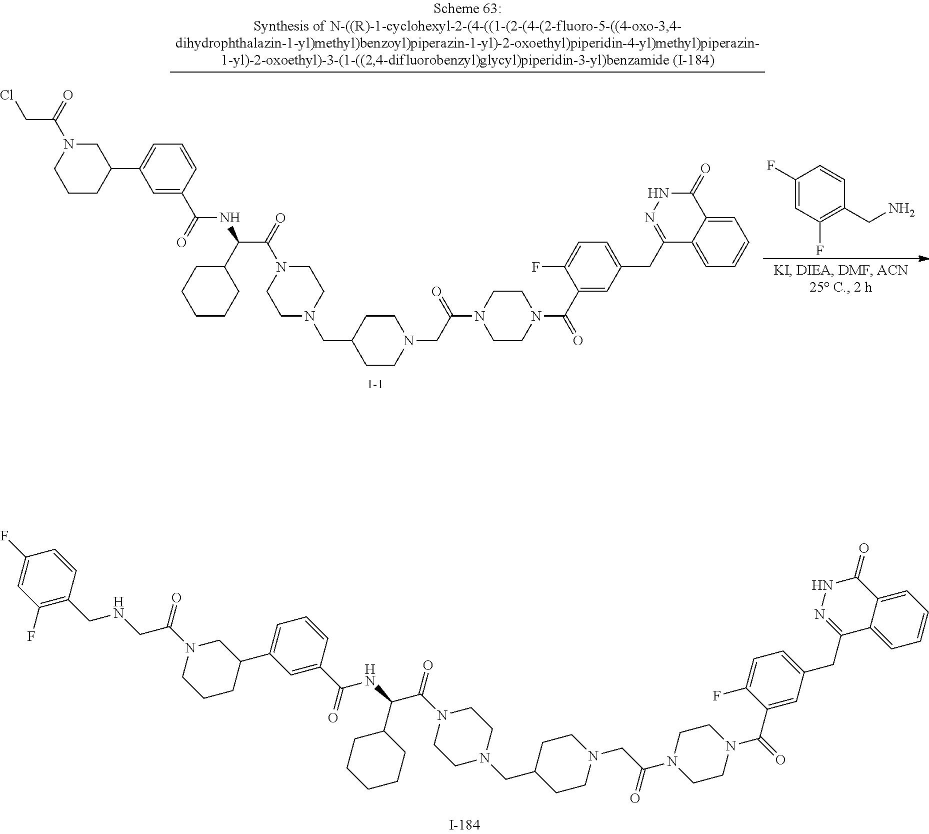 [SMILES: O=C(N[C@@H](C(=O)N1CCN(CC2CCN(CC(=O)N3CCN(C(=O)c4cc(Cc5n[nH]c(=O)c6ccccc56)ccc4F)CC3)CC2)CC1)C1CCCCC1)c1cccc(C2CCCN(C(=O)CCl)C2)c1 ; NCc1ccc(F)cc1F ; O=C(N[C@@H](C(=O)N1CCN(CC2CCN(CC(=O)N3CCN(C(=O)c4cc(Cc5n[nH]c(=O)c6ccccc56)ccc4F)CC3)CC2)CC1)C1CCCCC1)c1cccc(C2CCCN(C(=O)CNCc3ccc(F)cc3F)C2)c1]

To a solution of (2,4-difluorophenyl)methanamine (16.22 mg, 113.34 μmol, 2.5 eq) in ACN (0.5 mL) and DMF (0.5 mL) was added DIEA (23.44 mg, 181.34 μmol, 31.59 μL, 4 eq) and stirred at 25° C. for 1 hr. Then to the mixture was added KI (3.76 mg, 22.67 μmol, 0.5 eq) and Intermediate 1-1 (45 mg, 45.33 mol, 1 eq) and stirred at 25° C. for 2 hrs. The mixture was filtered and washed with MeOH (1 mL*3) to give the filtrate. The filtrate was concentrated to give a residue, which was purified by prep-HPLC (column: Waters Xbridge 150*25 mm*5 um; mobile phase: [water (ammonia hydroxide v/v)-ACN]; gradient: 35%-65% B over 10 min). The eluent was concentrated to remove MeCN and then lyophilized to afford I-184 (19.32 mg, 17.58 μmol, 38.77% yield, 100% purity) as a brown solid. Method E: Rt=0.417 min, [M+H]⁺=1099.5. SFC: Rt=3.472 min, 4.530 min. ¹H NMR (400 MHz, METHANOL-d₄) 6=8.37-8.30 (m, 1H), 7.98-7.92 (m, 1H), 7.85-7.80 (m, 2H), 7.75 (s, 1H), 7.73-7.68 (m, 1H), 7.51-7.35 (m, 5H), 7.16-7.10 (m, 1H), 6.99-6.88 (m, 2H), 4.91-4.90 (m, 1H), 4.60-4.49 (m, 1H), 4.38 (s, 2H), 3.88-3.64 (m, 10H), 3.60-3.45 (m, 5H), 3.27-3.07 (m, 4H), 2.93-2.67 (m, 4H), 2.53-2.32 (m, 4H), 2.21-2.20 (m, 2H), 2.13-2.00 (m, 3H), 1.91-1.66 (m, 10H), 1.62-1.49 (m, 2H), 1.33-1.06 (m, 8H). ¹⁹F NMR (377 MHz, METHANOL-d₄) δ=−113.4, −116.3, −120.7,

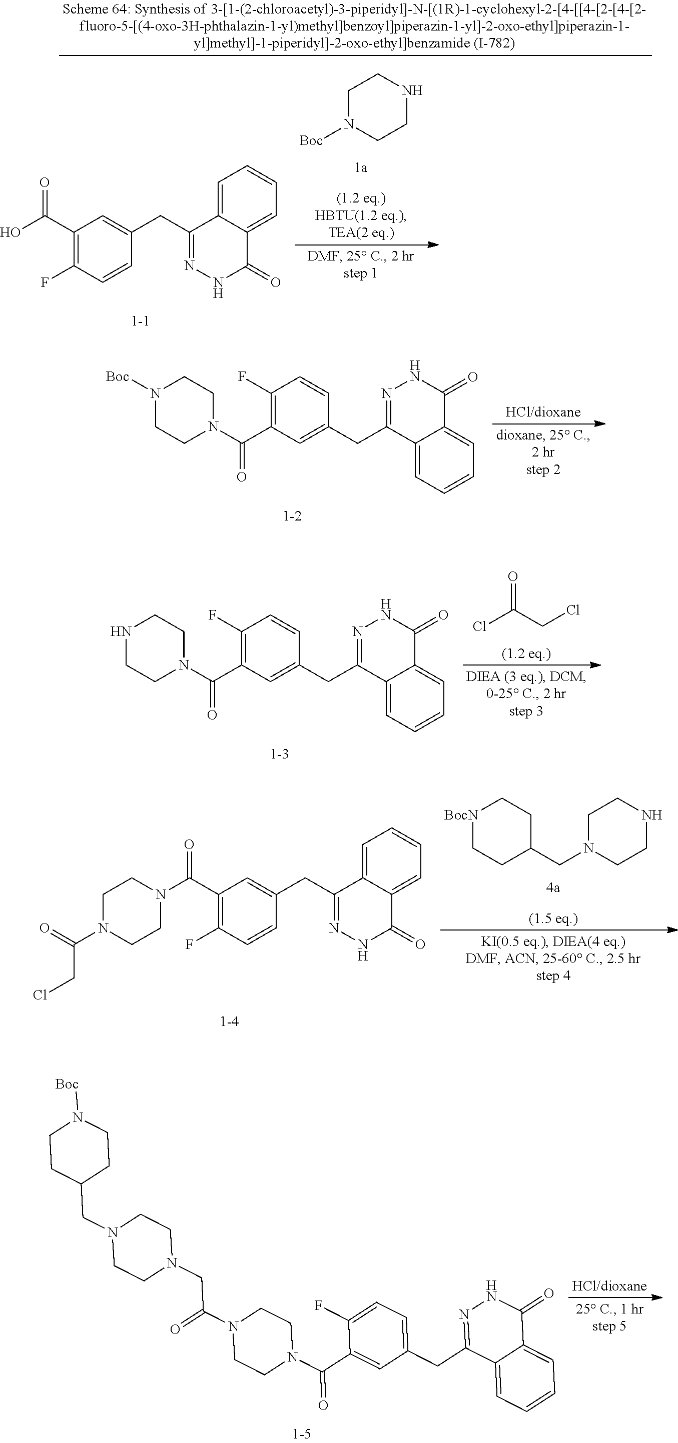

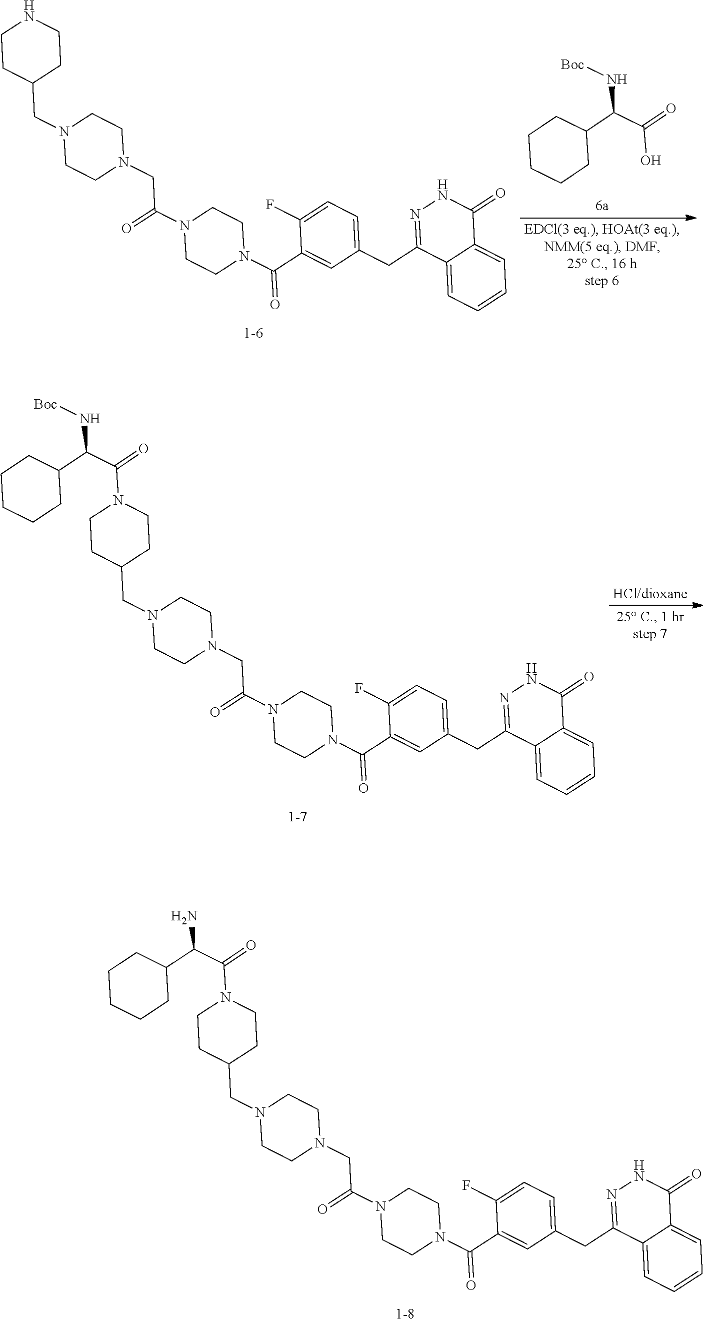

### Step 1: Synthesis of Intermediate 1-2

To a solution of Intermediate 1-1 (20 g, 67.05 mmol, 1 eq), tert-butyl piperazine-1-carboxylate (14.99 g, 80.46 mmol, 1.2 eq) in DMF (200 mL) was added HBTU (30.52 g, 80.46 mmol, 1.2 eq) and TEA (13.57 g, 134.11 mmol, 18.67 mL, 2 eq). The mixture was stirred at 25° C. for 2 hr. The reaction mixture was diluted with H₂O (200 mL) and filtered. And the filter cake was washed with DMF-H₂O (1:1, 2*40 ml), H₂O (2*40 ml), isopropanol (2*40 ml) and PE (2*40 ml) to give Intermediate 1-2 as a yellow solid. LCMS (Method G) Retention time: 0.580 min, [M+H]⁺=467.2. ¹H NMR (400 MHz, DMSO-d₆) δ=12.59 (s, 1H), 8.26 (dd, J=1.1, 7.8 Hz, 1H), 7.98-7.93 (m, 1H), 7.88 (dt, J=1.4, 7.6 Hz, 1H), 7.85-7.80 (m, 1H), 7.46-7.40 (m, 1H), 7.34 (dd, J=2.1, 6.4 Hz, 1H), 7.23 (t, J=9.0 Hz, 1H), 4.32 (s, 2H), 3.59 (br s, 2H), 3.23 (br s, 2H), 3.17-3.07 (m, 2H), 1.40 (s, 9H)

### Step 2: Synthesis of Intermediate 1-3

To Intermediate 1-2 (14 g, 30.01 mmol, 1 eq) was added HCl/dioxane (4 M, 140.00 mL). The reaction mixture was concentrated under reduced pressure to give Intermediate 1-3 (11.5 g, 28.55 mmol, 95.12% yield, HCl salt) as a brown solid. LCMS (Method E): Retention time: 0.355 min, [M+H]⁺=367.0.

### Step 3: Synthesis of Intermediate 1-4

To a solution of Intermediate 1-3 (12 g, 32.75 mmol, 1 eq) in DCM (120 mL) was added DIEA (12.70 g, 98.26 mmol, 17.11 mL, 3 eq) and 2-chloroacetyl chloride (4.44 g, 39.30 mmol, 3.13 mL, 1.2 eq) at 0° C. The mixture was stirred at 25° C. for 2 hr. The reaction mixture was washed with water (100 mL) and extracted with DCM (50 mL*2). The combined organic phase was washed with brine (100 mL*1), dried with anhydrous Na₂SO₄, filtered and concentrated in vacuum to give Intermediate 1-4 (13 g, 29.35 mmol, 89.62% yield) as a brown solid. LCMS (Method E): Retention time: 0.423 min, [M+H]⁺=443.0.

### Step 4: Synthesis of Intermediate 1-5

To a solution of tert-butyl 4-(piperazin-1-ylmethyl)piperidine-1-carboxylate (7.68 g, 27.10 mmol, 1 eq) in DMF (60 mL) and MeCN (50 mL) was added DIEA (14.01 g, 108.38 mmol, 18.88 mL, 4 eq), the reaction mixture was stirred at 25° C. for 0.5 h, then KI (2.25 g, 13.55 mmol, 0.5 eq) and Int-AA (12 g, 27.10 mmol, 1 eq) were added to the mixture, and the reaction mixture was stirred at 60° C. for 2 hr. The reaction mixture was washed with water (200 mL) and extracted with EA (150 mL*2). The combined organic phase was washed with brine (200 mL*1), and dried with anhydrous Na₂SO₄, filtered and concentrated in vacuum to give Intermediate 1-5 (18 g, 19.70 mmol, 72.71% yield, 75.5% purity) as a brown oil. LCMS (Method G): Retention time: 0.603 min, [M+H]⁺=690.5.

### Step 5: Synthesis of Intermediate 1-6

To a solution of Intermediate 1-5 (18 g, 26.09 mmol, 1 eq) in dioxane (80 mL) was added HCl/dioxane (4 M, 100 mL, 15.33 eq). The mixture was stirred at 25° C. for 1 hr. The reaction mixture was added to EA (20 mL) and stirred. The mixture was filtered and was concentrated under reduced pressure to give Intermediate 1-6 (16 g, 21.72 mmol, 83.24% yield, 85% purity, HCl salt) as a brown solid. LCMS (Method E): Retention time: 0.358 min, [M+H]⁺=590.3.

### Step 6: Synthesis of Intermediate 1-7

To a solution of Intermediate 1-6 (16 g, 27.13 mmol, 1 eq), (2R)-2-(tert-butoxycarbonylamino)-2-cyclohexyl-acetic acid (10.47 g, 40.70 mmol, 1.5 eq) in DMF (160 mL) was added EDCI (15.60 g, 81.40 mmol, 3 eq), NMM (13.72 g, 135.66 mmol, 14.91 mL, 5 eq) and HOAt (11.08 g, 81.40 mmol, 3 eq). The mixture was stirred at 25° C. for 15 hr. The reaction mixture was washed with water (50 mL) and extracted with DCM (40 mL*2). The combined organic phase was washed with brine (50 mL), dried with anhydrous Na₂SO₄, filtered and concentrated in vacuum to give Intermediate 1-7 (6 g, 7.01 mmol, 25.85% yield, 96.9% purity) as a brown solid. LCMS (Method G): Retention time: 0.645 min, [M+H]⁺=829.7. ¹H NMR (400 MHz, CHLOROFORM-d) δ=10.95-10.74 (m, 1H), 8.53-8.42 (m, 1H), 7.81-7.69 (m, 3H), 7.41-7.30 (m, 2H), 7.05 (br t, J=8.4 Hz, 1H), 5.39 (br d, J=8.9 Hz, 1H), 4.58 (br d, J=13.0 Hz, 1H), 4.47 (br d, J=4.8 Hz, 1H), 4.29 (s, 2H), 4.12 (q, J=7.1 Hz, 1H), 3.96 (br d, J=13.6 Hz, 1H), 3.75-3.56 (m, 4H), 3.35-2.96 (m, 6H), 2.61-2.37 (m, 8H), 2.26-2.02 (m, 4H), 1.90-1.53 (m, 14H), 1.27-1.01 (m, 9H).

### Step 7: Synthesis of Intermediate 1-8

To a solution of Intermediate 1-7 (6 g, 7.24 mmol, 1 eq) in dioxane (30 mL) was added HCl/dioxane (4 M, 30 mL, 16.58 eq). The mixture was stirred at 25° C. for 1 hr. The reaction mixture was concentrated under reduced pressure to give Intermediate 1-8 (5.5 g, 6.83 mmol, 94.33% yield, 95% purity, HCl salt) as an off-white solid. LCMS (Method E): Retention time (Method E): 0.737 min, [M+H]⁺=729.4.

### Step 8: Synthesis of Intermediate 1-10

To a solution of Intermediate 1-8 (2 g, 2.61 mmol, 1 eq, HCl salt), Intermediate 1-9 (1.20 g, 3.92 mmol, 1.5 eq) in DMF (20 mL) was added EDCI (1.50 g, 7.84 mmol, 3 eq), NMM (1.32 g, 13.07 mmol, 1.44 mL, 5 eq) and HOAt (1.07 g, 7.84 mmol, 3 eq). The mixture was stirred at 25° C. for 2 hr. The reaction mixture was diluted with 10 mL of H₂O and filtered. The filter cake was used for purification. The crude product was purified by reversed-phase HPLC (0.1% FA condition) to give Intermediate 1-10 (1.5 g, 1.46 mmol, 55.92% yield, 99% purity) as a yellow solid. LCMS (Method E) Retention time: 0.533 min, [M+H]⁺=1016.6.

### Step 9: Synthesis of Intermediate 1-11

To a solution of Intermediate 1-10 (1.5 g, 1.48 mmol, 1 eq) in dioxane (5 mL) was added HCl/dioxane (2 M, 20.00 mL, 27.10 eq). The mixture was stirred at 25° C. for 1 hr. The reaction mixture was concentrated under reduced pressure to give Intermediate 1-11 (1.4 g, 1.47 mmol, 99.57% yield, HCl salt) was obtained as an off-white solid. LCMS (Method E): Retention time: 0.417 min, [M+H]⁺=916.6.

### Step 10: Synthesis of I-782

To a solution of Intermediate 1-11 (1.4 g, 1.47 mmol, 1 eq, HCl salt) in DCM (14 mL) was added DIEA (569.83 mg, 4.41 mmol, 767.97 μL, 3 eq) and 2-chloroacetyl chloride (199.19 mg, 1.76 mmol, 140.47 L, 1.2 eq) at 0° C. The mixture was stirred at 25° C. for 2 hr. The reaction mixture was washed with water (20 mL) and extracted with EA (4 mL*2). The combined organic phase was washed with brine (15 mL), dried with anhydrous Na₂SO₄, filtered and concentrated in vacuum to give I-782 (900 mg, 879.49 μmol, 59.84% yield, 97% purity) as a yellow solid.

LCMS (Method E): Retention time: 0.475 min, [M+H]⁺=992.6. ¹H NMR (400 MHz, DMSO-d₆) δ =12.60 (s, 1H), 8.43 (br s, 1H), 8.26 (d, J=7.8 Hz, 1H), 8.00-7.93 (m, 1H), 7.88 (br t, J=7.5 Hz, 1H), 7.82 (br t, J=7.0 Hz, 2H), 7.76 (br t, J=6.2 Hz, 1H), 7.49-7.35 (m, 4H), 7.23 (br t, J=8.9 Hz, 1H), 4.79 (br s, 1H), 4.50-4.35 (m, 4H), 4.33 (s, 2H), 4.10 (q, J=5.2 Hz, 2H), 3.86 (br s, 1H), 3.69-3.54 (m, 3H), 3.48 (br d, J=18.6 Hz, 2H), 3.23-3.13 (m, 8H), 3.10-3.00 (m, 2H), 2.61 (br d, J=14.9 Hz, 3H), 2.37 (br s, 6H), 2.13-1.99 (m, 2H), 1.90 (br s, 2H), 1.76 (br s, 4H), 1.67 (br s, 3H), 1.59 (br d, J=2.8 Hz, 2H), 1.27-1.11 (m, 3H), 0.99 (br s, 3H), 0.91-0.81 (m, 1H).

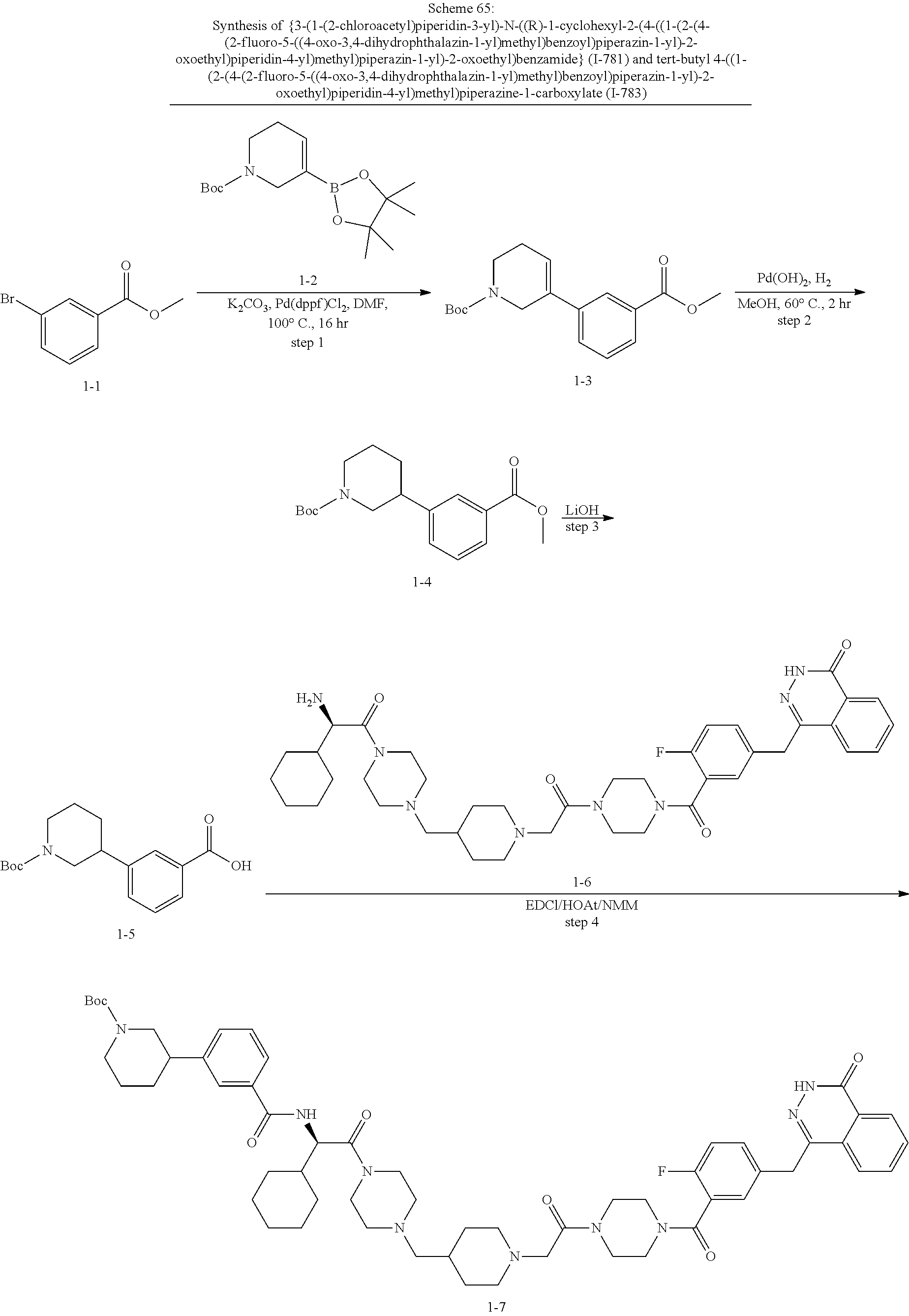

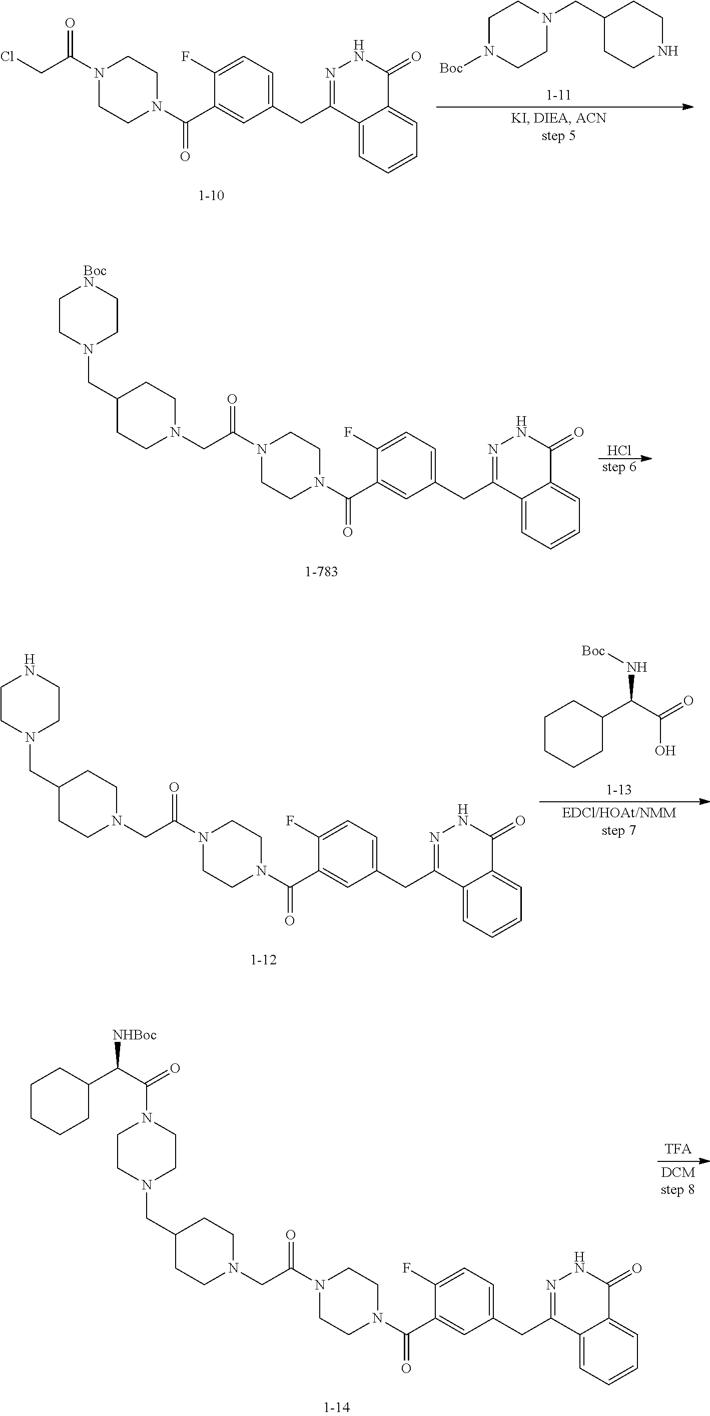

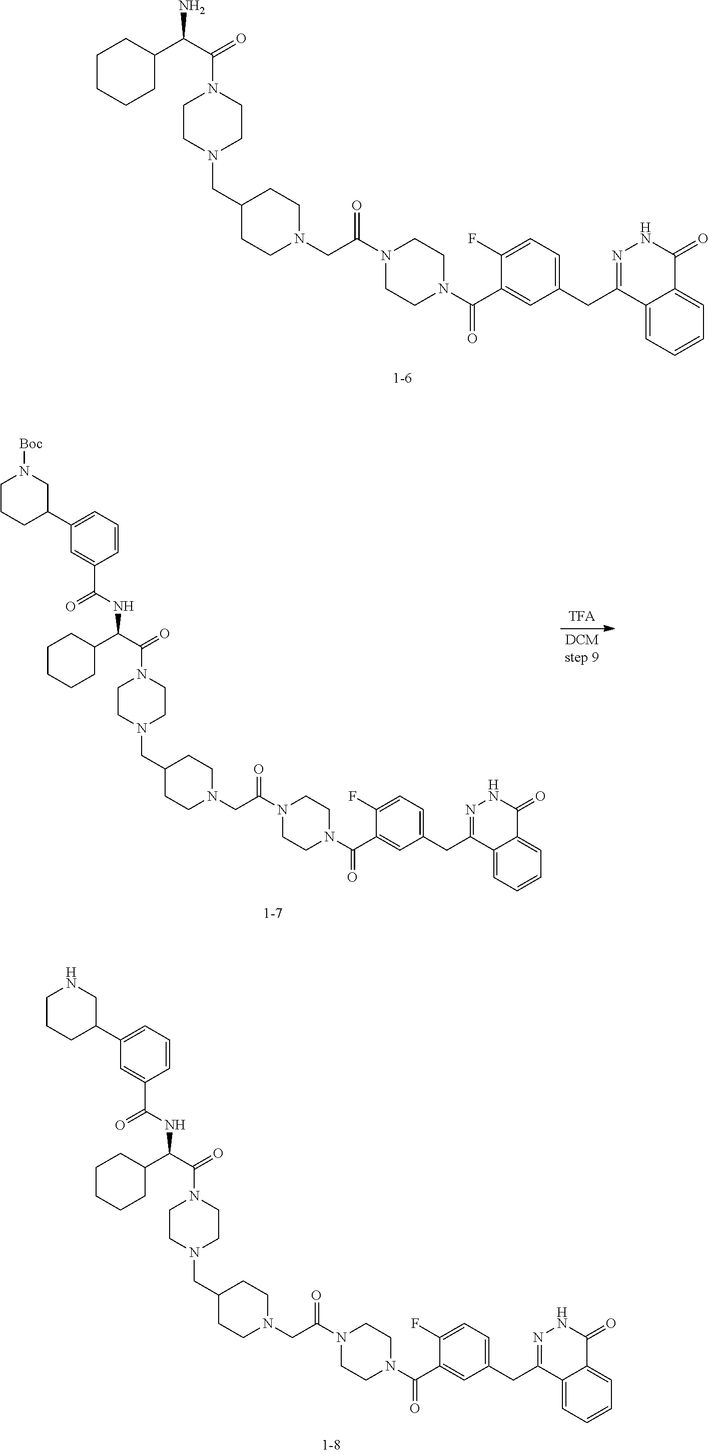 [SMILES: N[C@@H](C(=O)N1CCN(CC2CCN(CC(=O)N3CCN(C(=O)c4cc(Cc5n[nH]c(=O)c6ccccc56)ccc4F)CC3)CC2)CC1)C1CCCCC1 ; CC(C)(C)OC(=O)N1CCCC(c2cccc(C(=O)N[C@@H](C(=O)N3CCN(CC4CCN(CC(=O)N5CCN(C(=O)c6cc(Cc7n[nH]c(=O)c8ccccc78)ccc6F)CC5)CC4)CC3)C3CCCCC3)c2)C1 ; O=C(N[C@@H](C(=O)N1CCN(CC2CCN(CC(=O)N3CCN(C(=O)c4cc(Cc5n[nH]c(=O)c6ccccc56)ccc4F)CC3)CC2)CC1)C1CCCCC1)c1cccc(C2CCCNC2)c1]

### Step 1: Synthesis of Intermediate 1-3

To a solution of Intermediate 1-1 (5 g, 23.25 mmol, 1 eq), Intermediate 1-2 (10.78 g, 34.88 mmol, 1.5 eq), and K₂CO₃ (9.64 g, 69.75 mmol, 3 eq) in DMF (50 mL) was added Pd(dppf)Cl₂ (1.70 g, 2.33 mmol, 0.1 eq). Then the mixture was stirred at 100° C. for 16 hr. The reaction mixture was poured into water (200 mL) and extracted with EA (200 mL*2). The organic layer was washed with brine (200 mL) and dried over Na₂SO₄ and concentrated to give the crude product. The crude product was purified by flash silica gel chromatography (SiO₂, PE:EA=10:1 to 3:1) to give Intermediate 1-3 (7.3 g, 21.64 mmol, 93.09% yield, 94.1% purity) as a yellow solid. LCMS (Method E) LCMS: Retention time: 0.633 min, [M+Na]⁺=340.1. ¹H NMR (400 MHz, CHLOROFORM-d) δ=8.07 (s, 1H), 7.96 (d, J=7.6 Hz, 1H), 7.57 (br d, J=7.6 Hz, 1H), 7.49-7.39 (m, 1H), 6.37-6.25 (m, 1H), 4.31 (br s, 2H), 3.94 (s, 3H), 3.59-3.57 (m, 2H), 2.35 (br d, J=3.6 Hz, 2H), 1.52 (s, 9H).

### Step 2: Synthesis of Intermediate 1-4

To a solution of Pd(OH)₂ (700 mg, 20% purity) in MeOH (100 mL) was added Intermediate 1-3 (7.3 g, 23.00 mmol, 1 eq). Then the mixture was stirred at 60° C. for 2 hr under H₂ (15 psi). The mixture was filtered through a pad of Celite and washed with MeOH (30 mL*4) and the filtrate was concentrated to give Intermediate 1-4 (7.2 g, 21.28 mmol, 92.52% yield) as colorless oil. LCMS (Method E): Retention time: 0.626 min, [M+Na]⁺=342.2.

### Step 3: Synthesis of Intermediate 1-5

To a solution of Intermediate 1-4 (7.2 g, 22.54 mmol, 1 eq) in H₂O (70 mL) and MeOH (70 mL) was added LiOH·H₂O (2.84 g, 67.63 mmol, 3 eq). Then the mixture was stirred at 25° C. for 1 hr. The reaction mixture was poured into water (200 mL) and extracted with EA (200 mL), the aqueous phase was adjusted to pH=7 with 1 N HCl, and then extracted with EA (200 mL*2), the organic layer was washed with brine and dried over Na₂SO₄ and Concentrated to give Intermediate 1-5 (6.5 g, 20.04 mmol, 88.92% yield, 94.17% purity) as white solid. LCMS (Method E): Retention time: 0.555 min, [M+23]⁺=328.3. ¹H NMR (400 MHz, CHLOROFORM-d) δ=7.95-7.77 (m, 2H), 7.48-7.30 (m, 2H), 4.25-4.05 (m, 2H), 2.70 (br s, 2H), 2.01-1.95 (m, 2H), 1.77-1.47 (m, 3H), 1.41 (s, 9H).

### Step 4: Synthesis of Intermediate 1-7

To a solution of Intermediate 1-5 (1.05 g, 3.44 mmol, 1 eq), HOAt (468.01 mg, 3.44 mmol, 1 eq), EDCI (1.32 g, 6.88 mmol, 2 eq) and NMM (1.74 g, 17.19 mmol, 1.89 mL, 5 eq) in DMF (15 mL) was added Intermediate 1-6 (2.51 g, 3.44 mmol, 1 eq). The mixture was stirred at 25° C. for 1 h. The mixture was poured onto H₂O (60 mL) and filtered. The filter cake was washed with H₂O (10 mL*4) and concentrated under vacuum to give Intermediate 1-7 (4 g, crude) as a yellow solid. LCMS (Method E): Retention time: 0.481 min, [M+H]⁺=1016.9, SFC: Rt=0.760 min. ¹H NMR (400 MHz, DMSO-d₆) δ=12.59 (s, 1H), 8.49 (br d, J=7.9 Hz, 1H), 8.26 (br d, J=7.5 Hz, 1H), 7.96-7.73 (m, 5H), 7.48-7.33 (m, 4H), 7.23 (brt, J=8.7 Hz, 1H), 4.75 (brt, J=8.2 Hz, 1H), 4.33 (br s, 2H), 4.05-3.92 (m, 2H), 3.65-3.47 (m, 8H), 3.19-3.14 (m, 2H), 2.79 (br d, J=3.1 Hz, 1H), 2.66 (br d, J=10.9 Hz, 1H), 2.36-2.21 (m, 4H), 2.11-1.86 (m, 6H), 1.80-1.54 (m, 10H), 1.40 (br s, 10H), 1.28-0.93 (m, 11H), 0.84 (br d, J=7.5 Hz, 3H).

### Step 5: Synthesis of I-783

To a solution of Intermediate 1-10 (6 g, 13.55 mmol, 1 eq) in DMF (30 mL) and ACN (30 mL) was added KI (2.25 g, 13.55 mmol, 1 eq) and DIEA (7.00 g, 54.19 mmol, 9.44 mL, 4 eq) and Intermediate 1-11 (4.22 g, 14.90 mmol, 1.1 eq). The mixture was stirred at 60° C. for 1 h. The reaction mixture was filtered. The crude product was purified by reversed-phase HPLC (0.1% NH₃·H₂O condition) to give I-783 (8 g, 11.47 mmol, 84.65% yield, 98.894% purity) as a gray solid. LCMS (Method E): Retention time: 0.614 min, [M+H]⁺=690.4. ¹H NMR (400 MHz, DMSO-d₆) δ=12.76-12.42 (m, 1H), 8.27 (m, 1H), 8.05-7.78 (m, 3H), 7.55-7.20 (m, 3H), 4.33 (br s, 2H), 3.68-3.50 (m, 4H), 3.18-3.03 (m, 4H), 2.94-2.72 (m, 3H), 2.31-1.92 (m, 11H), 1.70-1.55 (m, 3H), 1.39 (br s, 10H), 1.15-0.85 (m, 3H).

### Step 6: Synthesis of Intermediate 1-12

To a solution of I-783 (4 g, 5.80 mmol, 1 eq) in dioxane (20 mL) was added HCl/dioxane (2 M, 20.00 mL, 6.90 eq). The mixture was stirred at 25° C. for 3 h. The mixture was concentrated under vacuum to give Intermediate 1-12 (3.7 g, crude, HCl salt) as a gray solid. LCMS (Method E): Retention time: 0.347 min, [M+H]⁺=590.4.

### Step 7: Synthesis of Intermediate 1-14

To a solution of Intermediate 1-13 (1.5 g, 5.83 mmol, 1 eq), HOAt (793.42 mg, 5.83 mmol, 1 eq), EDCI (2.23 g, 11.66 mmol, 2 eq) and NMM (2.95 g, 29.15 mmol, 3.20 mL, 5 eq) in DMF (15 mL) was added Intermediate 1-12 (3.65 g, 5.83 mmol, 1 eq, HCl salt). The mixture was stirred at 25° C. for 1 h. The reaction was poured to water (100 mL), extracted with EA (40 mL*3), and the combined organic layer was washed with brine (40 mL*3), dried over anhydrous Na₂SO₄, filtered and concentrated under vacuum to give a residue. The residue was triturated with 100 mL of PE:EA=10:1 at 25° C. for 30 min filtered and the filter cake was concentrated under vacuum to give Intermediate 1-14 (4.3 g, 5.19 mmol, 88.98% yield) as a gray solid. LCMS (Method E): Retention time: 0.665 min, [M+H]⁺=829.7. SFC: Rt=2.280 min. ¹H NMR (400 MHz, DMSO-d₆) δ=12.60 (s, 1H), 8.27 (br d, J=7.6 Hz, 1H), 8.01-7.95 (m, 1H), 7.93-7.80 (m, 2H), 7.50-7.34 (m, 2H), 7.24 (br t, J=8.9 Hz, 1H), 6.73 (br d, J=8.6 Hz, 1H), 4.34 (s, 2H), 4.21 (br t, J=7.8 Hz, 1H), 3.61-3.50 (m, 4H), 3.20-3.07 (m, 4H), 2.80 (br d, J=12.6 Hz, 1H), 2.37-2.21 (m, 4H), 2.14-2.04 (m, 2H), 2.01-1.93 (m, 2H), 1.74-1.49 (m, 9H), 1.36 (s, 9H), 1.31-1.29 (m, 1H), 1.30-1.23 (m, 1H), 1.17-1.00 (m, 6H), 0.99-0.90 (m, 2H), 0.88-0.80 (m, 4H).

### Step 8: Synthesis of Intermediate 1-6

To a solution of Intermediate 1-14 (4.3 g, 5.19 mmol, 1 eq) in DCM (35 mL) was added TFA (7 mL) at 0° C. Then the mixture was stirred at 25° C. for 3 h. The reaction was poured into sat. NaHCO₃ (200 mL), extracted with DCM (100 mL*3), and the combined organic layer was washed with brine (100 mL*3), dried over anhydrous Na₂SO₄, filtered and concentrated under vacuum to give Intermediate 1-6 (2.8 g, crude) as a yellow solid. LCMS (Method E): Retention time: 0.532 min, [M+H]⁺=729.4.

### Step 9: Synthesis of Intermediate 1-8

To a solution of Intermediate 1-7 (4 g, 3.94 mmol, 1 eq) in DCM (30 mL) was added TFA (6 mL) at 0° C. The mixture was stirred at 25° C. for 2 h. The reaction was poured into sat. NaHCO₃ (200 mL), extracted with DCM (100 mL*3), and the combined organic layer was washed with brine (100 mL*3), dried over anhydrous Na₂SO₄, filtered and concentrated under vacuum to give Intermediate 1-8 (3.3 g, crude) as a yellow solid. LCMS (Method E): Retention time: 0.377 min, [M+H]⁺=916.7.

### Step 10: Synthesis of I-781

To a solution of Intermediate 1-8 (3 g, 3.27 mmol, 1 eq) in DCM (30 mL) was added Intermediate 1-9 (443.81 mg, 3.93 mmol, 312.99 μL, 1.2 eq) and TEA (994.07 mg, 9.82 mmol, 1.37 mL, 3 eq). The mixture was stirred at 25° C. for 1 h. The reaction was poured into H₂O (100 ml), extracted with DCM (50 ml*3), and the combined organic layer was washed with brine (50 ml*3), dried over anhydrous Na₂SO₄, filtered and concentrated under vacuum to give a residue. The residue was purified by reversed-phase HPLC (0.1% NH₃·H₂O). I-781 (1.89 g, 1.87 mmol, 57.05% yield, 98.111% purity) was obtained as a gray solid. LCMS (Method G): Rt=0.443 min, [M+H]⁺=992.8 SFC: Rt=3.457 min, Rt=3.895 min. ¹H NMR (400 MHz, DMSO-d6) δ=12.59 (s, 1H), 8.47 (m, 1H), 8.27 (d, J=7.6 Hz, 1H), 8.00-7.95 (m, 1H), 7.93-7.87 (m, 1H), 7.86-7.74 (m, 3H), 7.48-7.34 (m, 4H), 7.24 (m, 1H), 4.76 (m, 1H), 4.50-4.32 (m, 5H), 3.97-3.81 (m, 1H), 3.70-3.46 (m, 9H), 3.21-3.06 (m, 5H), 2.83-2.62 (m, 4H), 2.35-2.22 (m, 4H), 2.19-2.03 (m, 4H), 2.00-1.87 (m, 4H), 1.82-1.57 (m, 10H), 1.45 (m, 1H), 1.19-0.99 (m, 6H).

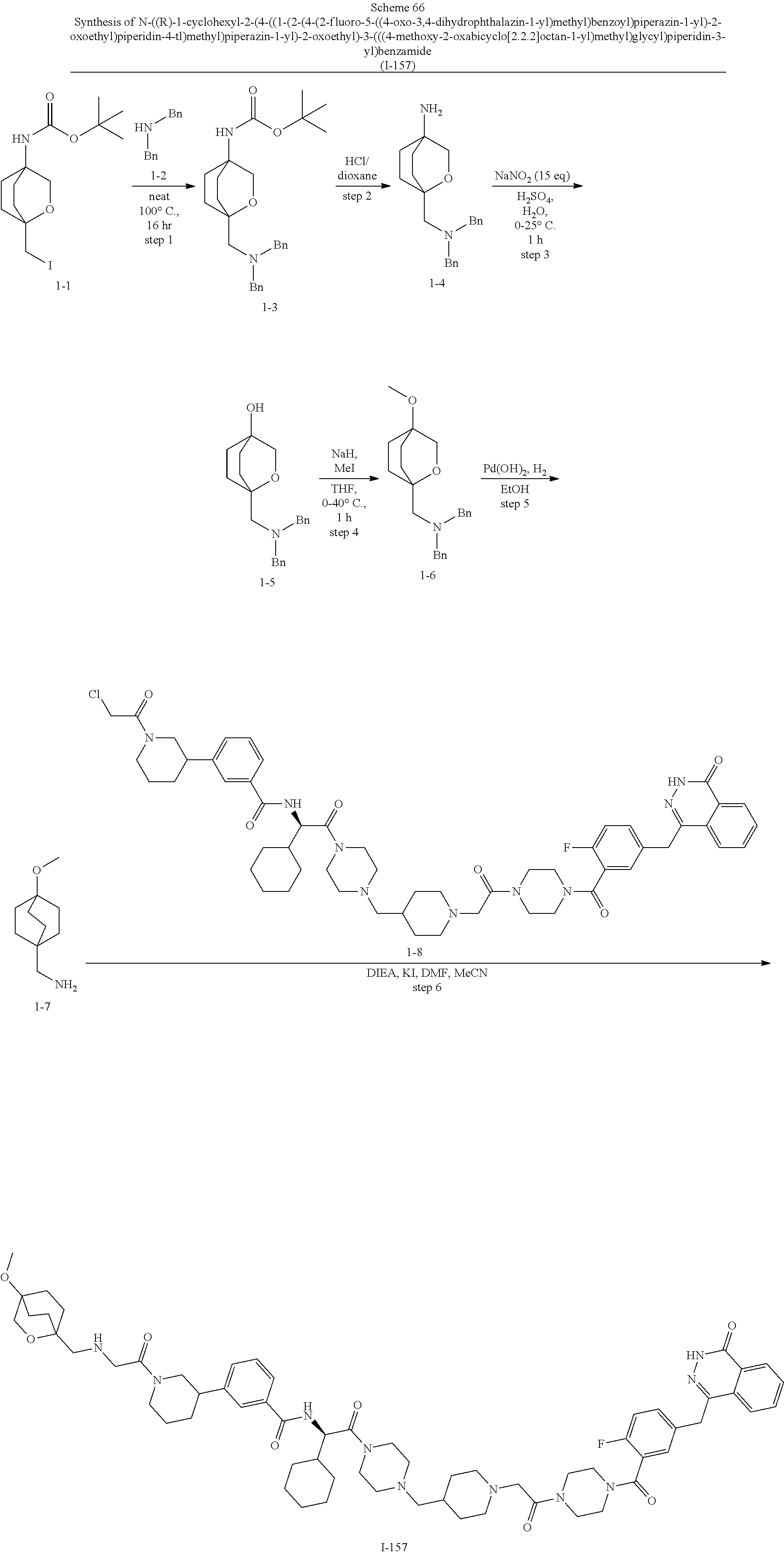

### Step 1: Synthesis of Intermediate 1-3

A solution of Intermediate 1-1 (4.1 g, 11.16 mmol, 1 eq) in Intermediate 1-2 (20 mL) was stirred at 100° C. for 16 hrs. The reaction mixture was concentrated under vacuum to give the crude product. The crude product was purified by prep-HPLC (column: Kromasil Eternity XT 250*80 mm*10 um; mobile phase: [water (NH₃H₂O)-ACN]; gradient: 80%-100% B over 30 min) to give Intermediate 1-3 (1 g, 2.10 mmol, 18.79% yield) as colorless oil. LCMS (Method E): Rt=0.519 min, [M+H]⁺=437.3

### Step 2: Synthesis of Intermediate 1-4

A solution of Intermediate 1-3 (1 g, 2.29 mmol, 1 eq) in HCl/dioxane (5 mL) was stirred at 25° C. for 1 hr. The reaction mixture was concentrated under vacuum to give Intermediate 1-4 (900 mg, 2.07 mmol, 90.30% yield, HCl) as colorless oil. LCMS (Method E): Rt=0.378 min, [M+H]⁺=337.3.

### Step 3: Synthesis of Intermediate 1-5

To a solution of Intermediate 1-4 (900 mg, 2.41 mmol, 1 eq, HCl) in H₂SO₄ (5 mL) was added NaNO₂ (2.50 g, 36.20 mmol, 15 eq) in H₂O (2.5 mL) at 0° C. Then the mixture was stirred at 25° C. for 1 hr. The reaction mixture was poured into water (5 mL) and extracted with EA (5 mL*2), the organic layer was washed with brine (5 mL) and dried over Na₂SO₄. The organics were concentrated to give the crude product. The crude product was purified by reverse phase column (FA) to give Intermediate 1-5 (420 mg, 1.20 mmol, 49.61% yield) as colorless oil. LCMS (Method E): Rt=0.429 min, [M+H]⁺=338.2.

### Step 4: Synthesis of Intermediate 1-6

To a mixture of Intermediate 1-5 (420 mg, 1.24 mmol, 1 eq) in THF (5 mL) was added NaH (74.67 mg, 1.87 mmol, 60% purity, 1.5 eq) at 0° C. and the mixture was stirred at 0° C. for 0.5 hr, then Mel (264.99 mg, 1.87 mmol, 116.22 μL, 1.5 eq) was added to the mixture and it was stirred at 40° C. for 1 hr. The reaction mixture was poured into H₂O (10 mL) and extracted with EtOAc (10 mL×3), the combined organic layers were dried over Na₂SO₄, filtered and concentrated to give a residue. The residue was purified by reverse phase column (FA) to give Intermediate 1-6 (400 mg, 1.11 mmol, 89.33% yield) as colorless oil. LCMS (Method E): Rt=0.453 min, [M+H]⁺=352.3.

### Step 5: Synthesis of Intermediate 1-7

To a solution of Intermediate 1-6 (400 mg, 1.14 mmol, 1 eq) in EtOH (4 mL) was added Pd(OH)₂ (199.78 mg, 284.51 μmol, 20% purity). Then the mixture was stirred at 25° C. for 16 hrs under H₂ (15 psi). The reaction mixture was filtered and the filtrate was concentrated under vacuum to give Intermediate 1-7 (200 mg, 1.05 mmol, 92.37% yield) as colorless oil. ¹H NMR (400 MHz, CHLOROFORM-d) δ=3.77-3.73 (m, 2H), 3.24 (s, 3H), 2.60 (s, 2H), 2.05-1.85 (m, 4H), 1.84-1.75 (m, 2H), 1.69-1.55 (m, 2H).

### Step 6: Synthesis of I-157

To a solution of Intermediate 1-7 (25.88 mg, 151.12 μmol, 2.5 eq) in DMF (1 mL) and MeCN (1 mL) was added DIEA (23.44 mg, 181.34 μmol, 31.59 μL, 3 eq) and stirred at 25° C. for 1 hr. Then to the mixture was added KI (5.02 mg, 30.22 μmol, 0.5 eq) and Intermediate 1-8 (60 mg, 60.45 μmol, 1 eq) and stirred at 25° C. for 15 hr. The reaction mixture was filtered to give the filtrate. The filtrate was purified by prep-HPLC (column: C18 150×30 mm; mobile phase: [water (FA)-ACN]; gradient: 18%-48% B over 7 min) to give I-157 (13.73 mg, 11.70 μmol, 19.36% yield, FA) as a white solid by lyophilization. LCMS (Method E): Rt=0.424 min, [M+H]⁺=1127.6. SFC: Rt=4.904 min, 7.111 min. ¹H NMR (400 MHz, METHANOL-d₄) δ=8.39 (d, J=7.6 Hz, 1H), 8.02-7.70 (m, 5H), 7.58-7.33 (m, 4H), 7.19-7.17 (m, 1H), 4.93 (br d, J=8.4 Hz, 1H), 4.72-4.49 (m, 2H), 4.40 (s, 2H), 3.92-3.79 (m, 3H), 3.78-3.66 (m, 8H), 3.60-3.50 (m, 3H), 3.46-3.35 (m, 3H), 3.27-3.16 (m, 4H), 3.10-2.97 (m, 2H), 2.95-2.66 (m, 4H), 2.58-2.36 (m, 4H), 2.35-2.18 (m, 4H), 2.10-1.86 (m, 9H), 1.85-1.55 (m, 12H), 1.43-1.19 (m, 6H), 1.19-1.05 (m, 2H).

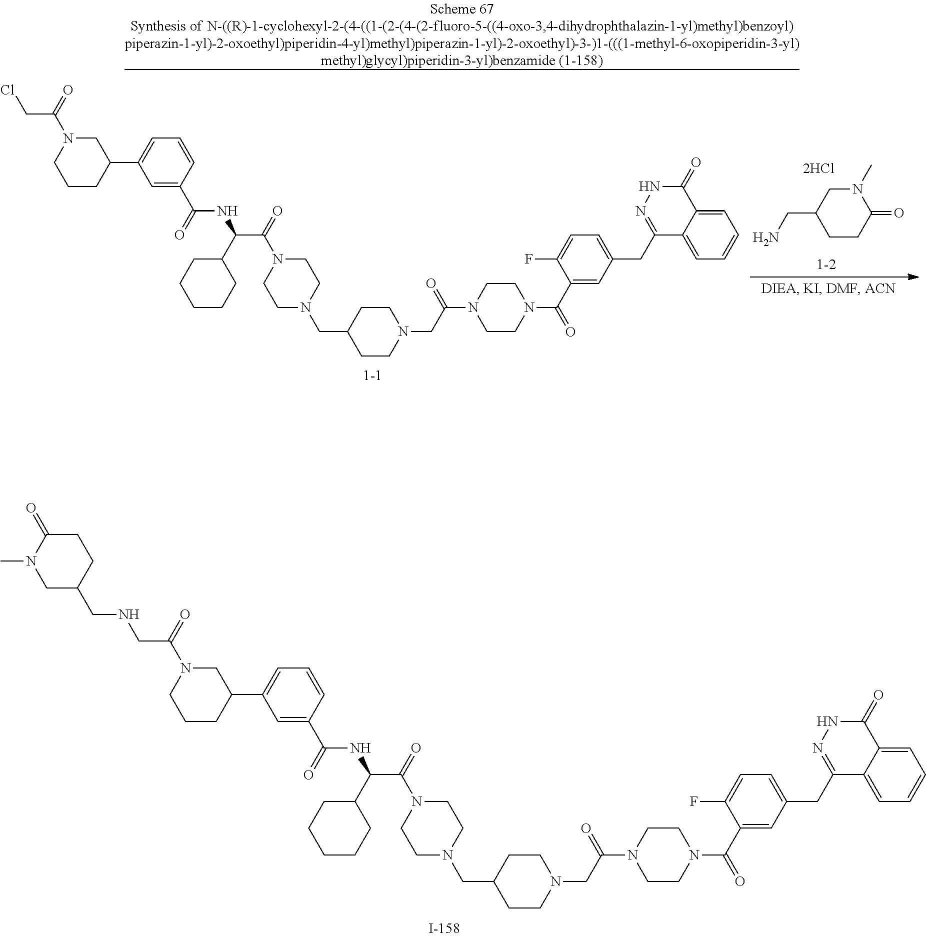

To a solution of Intermediate 1-2 (32.51 mg, 151.12 μmol, 2.5 eq, 2HCl) in MeCN (1 mL) and DMF (1 mL) was added DIEA (39.06 mg, 302.23 μmol, 52.64 μL, 5 eq) and stirred at 25° C. for 1 hr. Then to the mixture was added KI (5.02 mg, 30.22 μmol, 0.5 eq) and Intermediate 1-1 (60 mg, 60.45 μmol, 1 eq) and stirred at 25° C. for 15 hrs. The reaction mixture was filtered to give the filtrate. The filtrate was purified by prep-HPLC (column: C18 150×30 mm; mobile phase: [water (FA)-ACN]; gradient: 10%-40% B over 7 min) to give I-158 (16.52 mg, 14.44 μmol, 23.88% yield, FA salt) as a yellow solid. LCMS (Method B): Retention time: 0.506 min, [M+H]⁺=1098.7 LCMS (Method E): Retention time (Method E): 0.399 min, [M+H]⁺=1098.6 SFC: Retention time: 4.561 min, 5.778 min. ¹H NMR (400 MHz, METHANOL-d₄) δ=8.39 (d, J=7.2 Hz, 1H), 8.04-7.67 (m, 5H), 7.58-7.34 (m, 4H), 7.19-7.17 (m, 1H), 4.61 (br d, J=10.0 Hz, 1H), 4.41 (s, 2H), 3.96-3.63 (m, 10H), 3.62-3.45 (m, 6H), 3.37 (br s, 2H), 3.26-3.06 (m, 4H), 2.95 (d, J=8.8 Hz, 3H), 2.91-2.66 (m, 4H), 2.58-2.31 (m, 8H), 2.27-2.25 (m, 2H), 2.20-2.05 (m, 2H), 2.01-1.52 (m, 14H), 1.46-1.04 (m, 8H).

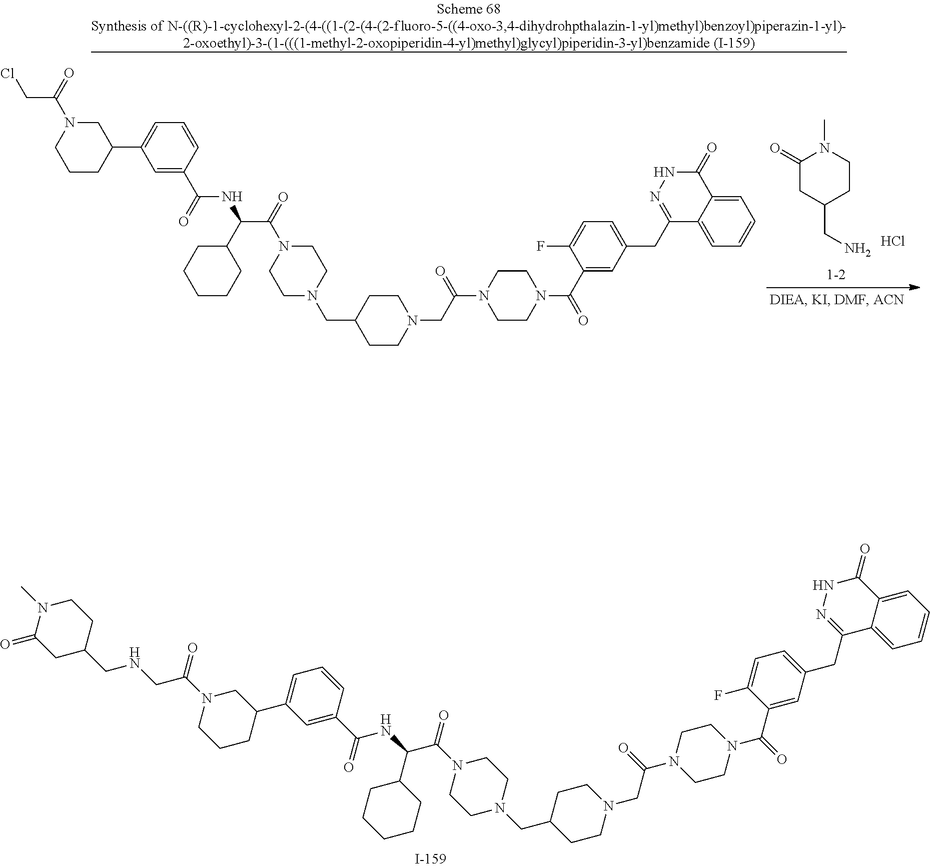

To a solution of Intermediate 1-2 (27.00 mg, 151.12 μmol, 2.5 eq, HCl) in MeCN (1 mL) and DMF (1 mL) was added DIEA (31.25 mg, 241.79 μmol, 42.11 μL, 4 eq) and the mixture was stirred at 25° C. for 1 hr. Then to the mixture was added KI (5.02 mg, 30.22 μmol, 0.5 eq) and Intermediate 1-1 (60 mg, 60.45 μmol, 1 eq) and stirred at 25° C. for 15 hrs. The reaction mixture was filtered to give the filtrate. The filtrate was purified by prep-HPLC (column: C18 150×30 mm; mobile phase: [water (FA)-ACN]; gradient: 10%-40% B over 7 min) to give I-159 (18.59 mg, 16.93 μmol, 28% yield) as a yellow solid. LCMS (Method E): Retention time: 0.398 min, [M+H]⁺=1098.7. SFC: Retention time: 7.388 min, 10.357 min. ¹H NMR (400 MHz, METHANOL-d₄) δ=8.39 (br d, J=7.6 Hz, 1H), 8.06-7.67 (m, 5H), 7.61-7.33 (m, 4H), 7.19-7.17 (m, 1H), 4.66-4.54 (m, 1H), 4.40 (s, 2H), 3.92-3.64 (m, 10H), 3.61-3.49 (m, 4H), 3.48-3.34 (m, 5H), 3.28-3.16 (m, 1H), 3.09-3.07 (m, 2H), 2.95 (d, J=4.0 Hz, 3H), 2.90-2.63 (m, 4H), 2.59-2.22 (m, 9H), 2.19-2.00 (m, 4H), 1.99-1.76 (m, 8H), 1.75-1.51 (m, 5H), 1.45-1.02 (m, 8H).

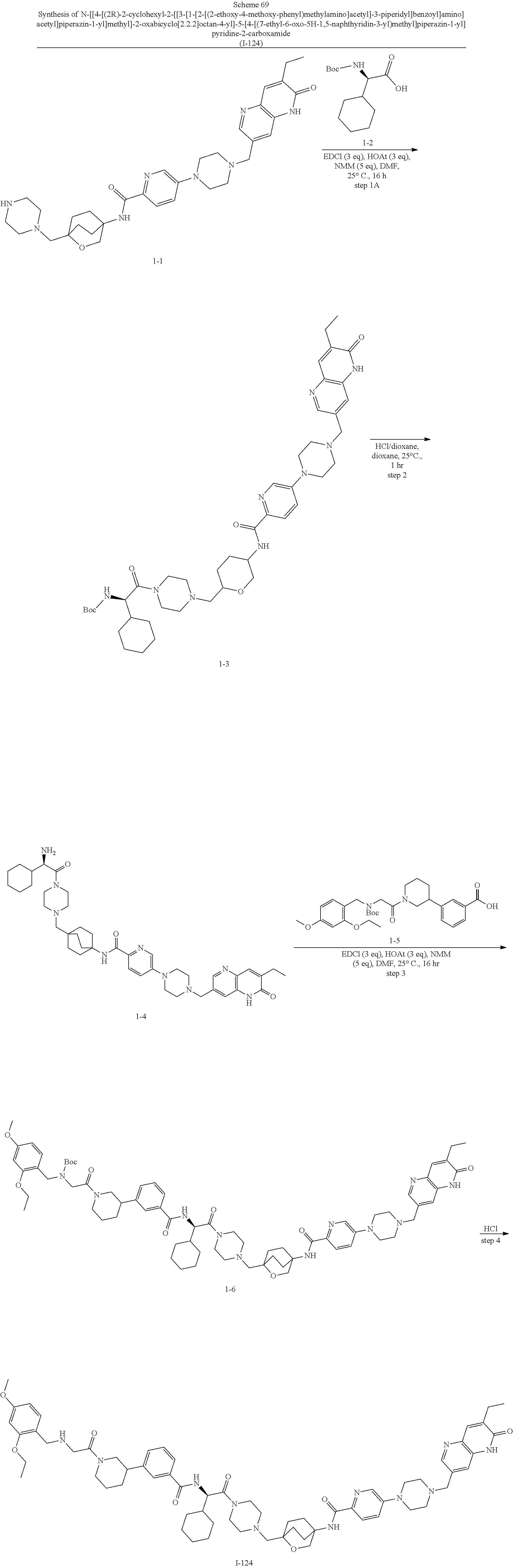

### Step 1: Synthesis of Intermediate 1-3

To a solution of Intermediate 1-2 (963.76 mg, 3.75 mmol, 1.5 eq), EDCI (1.44 g, 7.49 mmol, 3 eq) and HOAt (1.02 g, 7.49 mmol, 3 eq) in DMF (8 mL) was added Intermediate 1-1 (1.5 g, 2.50 mmol, 1 eq) and NMM (1.26 g, 12.48 mmol, 1.37 mL, 5 eq), and the mixture was stirred at 25° C. for 16 hr. The residue was diluted with H₂O (20 mL) and was extracted with ethyl acetate (25 mL*2). The combined organic phase was washed with brine (10 mL), dried over anhydrous Na₂SO₄, filtered and concentrated in vacuum. The crude residue was purified through flash silica gel chromatography (ISCO; 20 g SepaFlash® Silica Flash Column, eluent of 0˜10% DCM/MeOH) to give Intermediate 1-3 (500 mg, 559.48 μmol, 22.41% yield, 94% purity) as yellow oil. LCMS (Method G): Retention time: 0.723 min, [M+H]⁺=840.6.

### Step 2: Synthesis of Intermediate 1-4

To a solution of Intermediate 1-2 (450 mg, 535.67 μmol, 1 eq) in DCM (3 mL) was added HCl/dioxane (3 mL), the mixture was stirred at 25° C. for 1 hr. The resulting product was filtered and the filtrate was concentrated in vacuum. Intermediate 1-4 (400 mg, 489.43 μmol, 91.37% yield, 95% purity, HCl salt) was obtained as yellow solid. LCMS (Method G): Retention time: 0.639 min, [M+H]⁺=740.4.

### Step 3: Synthesis of Intermediate 1-6

To a solution of Intermediate 1-5 (305.22 mg, 579.59 μmol, 1.5 eq), EDCI (222.22 mg, 1.16 mmol, 3 eq) and HOAt (157.78 mg, 1.16 mmol, 3 eq) in DMF (2 mL) was added Intermediate 1-4 (300 mg, 386.39 μmol, 1 eq, HCl) and NMM (195.41 mg, 1.93 mmol, 212.41 μL, 5 eq), the mixture was stirred at 25° C. for 2 hr. The mixture was purified by reversed-phase HPLC (0.1% FA condition) to give Intermediate 1-6 (100 mg, 80.09 μmol, 20.73% yield, 100% purity) was obtained as white solid. LCMS (Method E): Retention time: 0.524 min, [M+H]⁺=1248.9.

### Step 4: Synthesis of I-124

To a solution of HCl (12 M, 4.04 μL, 1.01 eq) in dioxane (0.06 mL) and H₂O (0.6 mL) was added Intermediate 1-6 (60 mg, 48.06 μmol, 1 eq). The mixture was stirred at 40° C. for 2 hrs. The crude product was purified by reversed-phase HPLC (0.1% FA condition) to give I-124 (25.52 mg, 21.93 mol, 45.63% yield, 98.67% purity) as a yellow solid. LCMS (Method G): Retention time: 0.819 min, [M+H]⁺=1148.9. ¹H NMR (400 MHz, METHANOL-d4) δ=8.49 (d, J=1.6 Hz, 1H), 8.23 (d, J=2.8 Hz, 1H), 7.88-7.81 (m, 2H), 7.78-7.74 (m, 2H), 7.72 (m, 1H), 7.53-7.41 (m, 2H), 7.35 (dd, J=2.8, 8.8 Hz, 1H), 7.29 (t, J=8.0 Hz, 1H), 6.65-6.60 (m, 1H), 6.57 (m, 1H), 4.60-4.42 (m, 1H), 4.24-4.18 (m, 2H), 4.18-4.12 (m, 2H), 4.11-4.00 (m, 4H), 3.81 (d, J=4.0 Hz, 4H), 3.75 (s, 4H), 3.70 (m, 1H), 3.57 (m, 1H), 3.44-3.36 (m, 4H), 3.23 (br s, 1H), 2.81-2.57 (m, 12H), 2.39 (br s, 2H), 2.33-2.23 (m, 2H), 2.10-1.98 (m, 5H), 1.87 (br s, 4H), 1.77 (m, 4H), 1.73-1.65 (m, 3H), 1.59-1.36 (m, 4H), 1.28 (m, 6H), 1.18-1.08 (m, 2H). SFC: Retention time: 3.177 min. SFC: Retention time: 5.416 min.

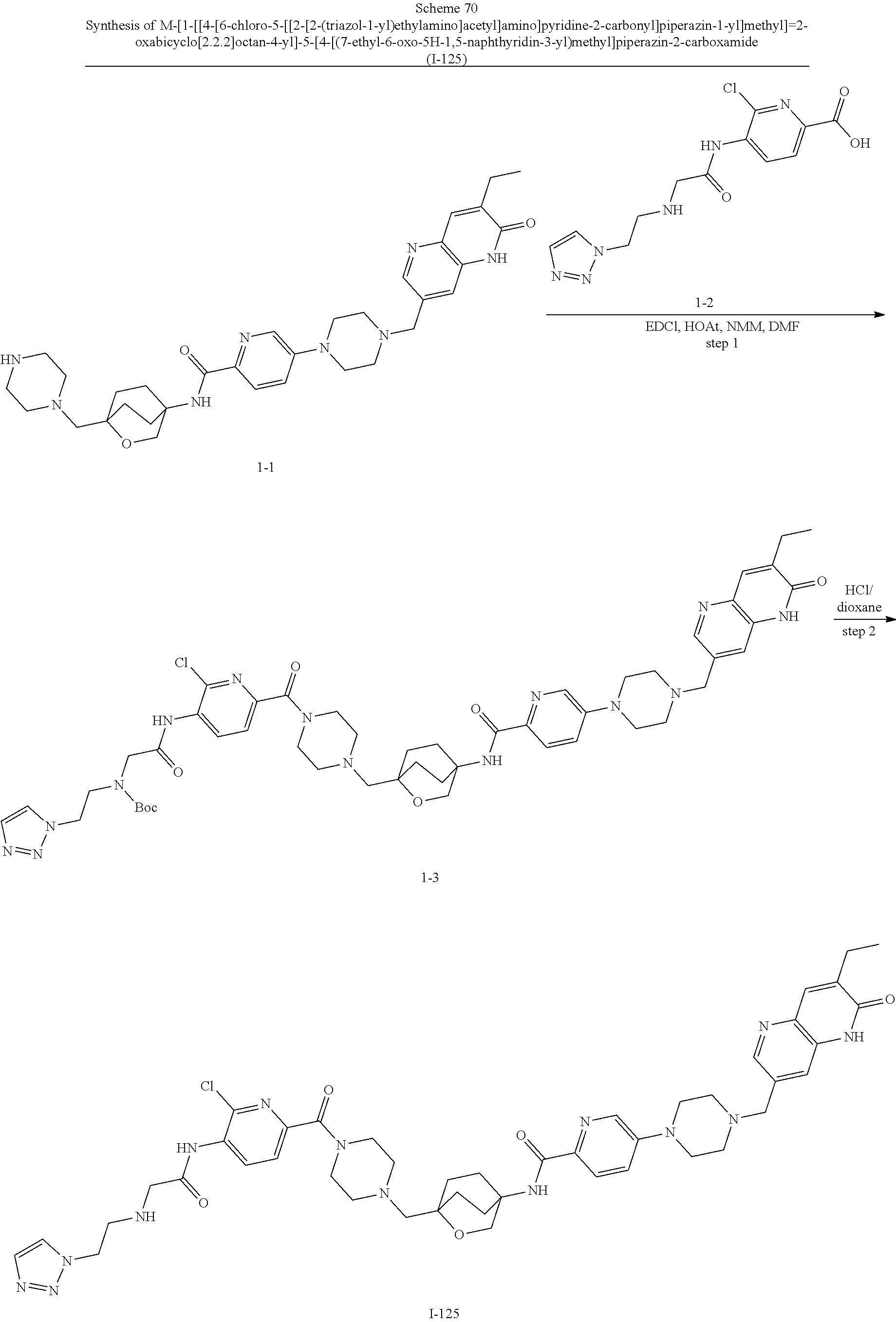

### Step 1: Synthesis of Intermediate 1-3

To a solution of Intermediate 1-2 (90 mg, 211.85 μmol, 1 eq), EDCI (81.22 mg, 423.69 μmol, 2 eq), HOAt (14.42 mg, 105.92 μmol, 0.5 eq), and NMM (107.14 mg, 1.06 mmol, 116.45 μL, 5 eq) in DMF (1 mL) was added Intermediate 1-1 (152.72 mg, 254.21 μmol, 1.2 eq). Then the mixture was stirred at 25° C. for 1 hr. The reaction mixture was poured into water (5 mL) and extracted with EA (5 mL*2), the organic layer was washed with brine (5 mL) and dried over Na₂SO₄. The organics were concentrated to give the crude product. The crude product was purified by flash silica gel chromatography (SiO₂, PE:EA=3:1 to 1:1) to give Intermediate 1-3 (80 mg, 69.39 μmol, 32.76% yield, 87.4% purity) as a white solid. LCMS (Method E): Rt=0.408 min, [M/2+H]⁺=504.7.

### Step 2: Synthesis of I-125

A solution of Intermediate 1-3 (65 mg, 64.51 μmol, 1 eq) in HCl/dioxane (0.5 mL) was stirred at 25° C. for 1 hr. The crude product was purified by prep-HPLC (column: Phenomenex luna C18 150*25 mm*10 um; mobile phase: [water (HCl)-ACN]; gradient: 1%-25% B over 10 min) to give I-125 (35.76 mg, 37.35 μmol, 57.90% yield, 98.59% purity, HCl) as a white solid by lyophilization. LCMS (Method E): Rt=0.359 min, [M+H]⁺=907.5. ¹H NMR (400 MHz, METHANOL-d₄) δ=8.81 (d, J=1.6 Hz, 1H), 8.64 (d, J=8.4 Hz, 1H), 8.42 (d, J=2.8 Hz, 1H), 8.29 (s, 1H), 8.23-8.15 (m, 2H), 7.93 (s, 1H), 7.88 (s, 1H), 7.85-7.80 (m, 2H), 4.96-4.90 (m, 4H), 4.71 (s, 2H), 4.65-4.53 (m, 1H), 4.43-4.31 (m, 1H), 4.30-4.23 (m, 4H), 3.95-3.70 (m, 7H), 3.68-3.52 (m, 5H), 3.35 (br s, 4H), 2.75-2.73 (m, 2H), 2.47-2.36 (m, 2H), 2.28-2.09 (m, 4H), 2.00-1.87 (m, 2H), 1.34-1.31 (m, 3H).

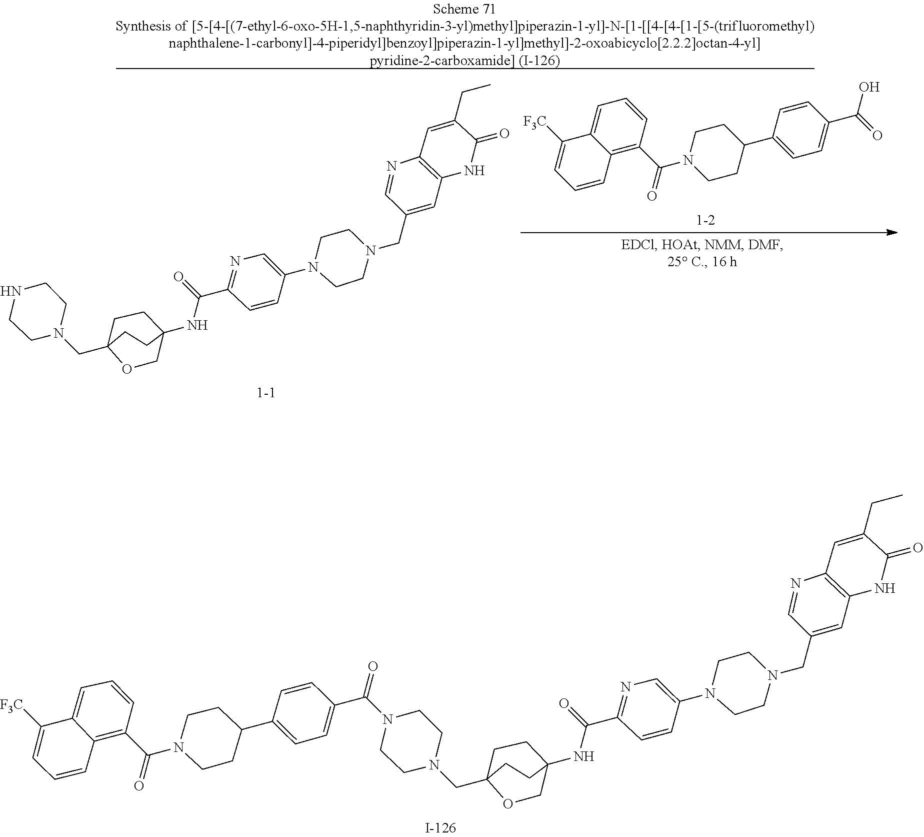

To a solution of Intermediate 1-2 (53 mg, 124.84 μmol, 1.5 eq) in DMF (1 mL) was added EDCI (48 mg, 249.69 μmol, 3 eq), HOAt (34 mg, 249.69 μmol, 3 eq), NMM (42 mg, 416.14 μmol, 45.75 μL, 5 eq) and Intermediate 1-1 (50 mg, 83.23 μmol, 1 eq,). The mixture was stirred at 25° C. for 12 hr. The reaction solution was filtered and concentrated. The residue was dissolved in DMF (2 mL) and purified by prep-HPLC (column: Phenomenex luna C18 150*25 mm*10 um; mobile phase: [water(FA)-ACN]; gradient: 20%-50% B over 9 min) to give I-126 (13.02 mg, 12.76 μmol, 15.33% yield, 99% purity) as a white solid. LCMS (Method E): Retention time: 0.480 min, [M+H]⁺=1010.6. ¹H NMR (400 MHz, METHANOL-d4) 6=8.48 (s, 1H), 8.44-8.39 (m, 1H), 8.30-8.19 (m, 3H), 8.05-7.99 (m, 1H), 7.88-7.81 (m, 2H), 7.79-7.67 (m, 4H), 7.58 (d, J=8.0 Hz, 1H), 7.43-7.29 (m, 6H), 5.07-4.94 (m, 1H), 4.59 (s, 1H), 4.08 (s, 2H), 3.73 (s, 4H), 3.50-3.36 (m, 7H), 3.24-3.03 (m, 2H), 2.99-2.88 (m, 1H), 2.66 (q, J=7.5 Hz, 7H), 2.54 (d, J=7.6 Hz, 2H), 2.36-2.21 (m, 4H), 2.14-1.96 (m, 5H), 1.95-1.83 (m, 1H), 1.80-1.65 (m, 4H), 1.54-1.39 (m, 1H), 1.28 (t, J=7.2 Hz, 3H).

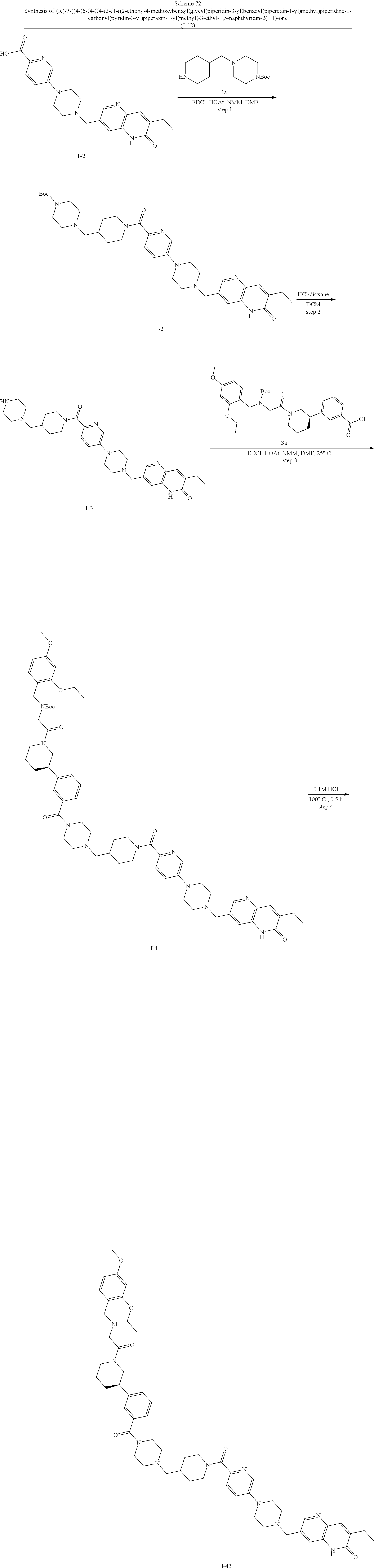

### Step 1: Synthesis of Intermediate 1-2

To a solution of Intermediate 1-1 (327.75 mg, 1.16 mmol, 1.3 eq) in DMF (5 mL) was added EDCI (511.61 mg, 2.67 mmol, 3 eq), HOAt (121.08 mg, 889.59 μmol, 1 eq) and NMM (899.80 mg, 8.90 mmol, 978.04 μL, 10 eq). The mixture was stirred at 25° C. for 1 hr. The mixture was poured into water (3 mL) and extracted with EA (3 mL*3). The combined organic layers were concentrated to give a residue. The residue was purified by flash silica gel chromatography (ISCO; 40 g SepaFlash Silica Flash Column, Eluent of 0˜40% MeOH/ethyl acetate gradient @60 mL/min) and the eluent was concentrated to give the product. Intermediate 1-2 (500 mg, 637.49 μmol, 71.66% yield, 84% purity) was obtained as white solid. LCMS (Method E): Rt=0.381 min, [M+H]⁺=659.3.

### Step 2: Synthesis of Intermediate 1-3

To a solution of Intermediate 1-2 (500 mg, 758.92 μmol, 1 eq) in DCM (3.5 mL) was added HCl/dioxane (4 M, 3.5 mL, 18.45 eq). The mixture was stirred at 25° C. for 0.5 hr. The reaction mixture was concentrated and used for the next step directly. The residue of Intermediate 1-3 (400 mg, 631.75 mol, 83.24% yield, 94% purity, HCl salt) was obtained as colorless liquid. LCMS (Method E): Rt=0.319 min, [M+H]⁺=559.4.

### Step 3: Synthesis of Intermediate 1-4

To a solution of Intermediate 1-3 (44.24 mg, 84.01 μmol, 1 eq) in DMF (0.5 mL) was added EDCI (48.31 mg, 252.03 μmol, 3 eq), HOAt (11.43 mg, 84.01 μmol, 11.75 μL, 1 eq) and NMM (84.97 mg, 840.09 mol, 92.36 μL, 10 eq). The mixture was stirred at 25° C. for 1 hr. The mixture was poured into water (3 mL) and extracted with EA (3 mL*3). The combined organic layers were concentrated to give a residue. The residue was purified by flash silica gel chromatography (ISCO; 12 g SepaFlash Silica Flash Column, Eluent of 0-40% MeOH/Ethyl acetate gradient @20 mL/min) and the eluent was concentrated to give Intermediate 1-4 (85 mg, 79.64 μmol, 94.80% yield) as a white solid. LCMS (Method E): Rt=0.484 min, [M+H]⁺=1067.7. SFC: Rt=1.505 min, ee value=100%.

### Step 4: Synthesis of I-42

To a solution of Intermediate 1-4 (80 mg, 74.95 μmol, 1 eq) was added HCl (0.1 M, 3.00 mL, 4 eq). The mixture was stirred at 100° C. for 0.5 hr. To the mixture was added NaHCO₃ to adjust pH to 9˜10, and then the mixture was lyophilized to give a residue. The product was purified by prep-HPLC (column: Waters Xbridge 150*25 mm*5 um; mobile phase: [water (NH₃H2O)-ACN]; gradient: 32%-62% B over 9 min) and the eluent was concentrated to remove MeCN and then lyophilized. I-42 (34.29 mg, 33.84 mol, 45.14% yield, 95.44% purity) was obtained as a white solid. LCMS (Method E): Rt=0.392 min, [M+H]⁺=967.5. SFC: Rt=2.183 min, ee value=100%. ¹H NMR (400 MHz, METHANOL-d₄) δ=8.48 (d, J=1.6 Hz, 1H), 8.26-8.20 (m, 1H), 7.86-7.74 (m, 2H), 7.50-7.35 (m, 4H), 7.33-7.23 (m, 2H), 7.12-7.00 (m, 1H), 6.53-6.41 (m, 2H), 4.65-4.46 (m, 2H), 4.05-4.00 (m, 2H), 3.91 (d, J=11.6 Hz, 1H), 3.84-3.67 (m, 10H), 3.50-3.34 (m, 8H), 3.15-3.03 (m, 2H), 2.92-2.81 (m, 1H), 2.76-2.62 (m, 8H), 2.53 (s, 2H), 2.40 (s, 2H), 2.27 (d, J=6.4 Hz, 2H), 2.05-1.97 (m, 1H), 1.94-1.73 (m, 5H), 1.65-1.51 (m, 1H), 1.42-1.40 (m, 3H), 1.28-1.20 (m, 3H), 1.25-1.14 (m, 2H).

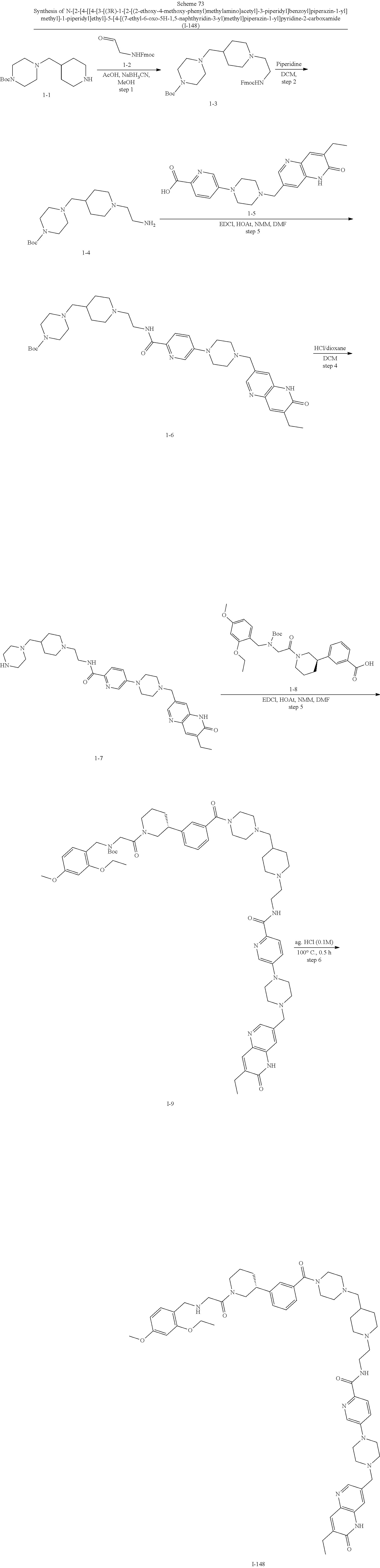

### Step 1: Synthesis of Intermediate 1-3

To a solution of Intermediate 1-1 (1 g, 3.53 mmol, 1 eq) and Intermediate 1-2 (1.49 g, 5.29 mmol, 1.5 eq) in MeOH (10 mL) was added AcOH (423.78 mg, 7.06 mmol, 403.99 μL, 2 eq), the mixture was stirred at 25° C. for 0.25 h, then NaBH₃CN (665.21 mg, 10.59 mmol, 3 eq) was added to the mixture, and the mixture was stirred at 25° C. for 1.75 hrs. The reaction mixture was quenched by addition of HCl (1 M, 10 mL), and then was diluted with DCM (10 mL) and extracted with DCM (10 mL*3). The combined organic layers were dried over anhydrous Na₂SO₄, filtered and concentrated under reduced pressure to give a residue. The residue was purified by reversed phase chromatography (0.1% FA condition), then concentrated to remove organic solvents and lyophilized to give Intermediate 1-3 (1 g, 1.79 mmol, 50.62% yield, 98% purity) as a white solid. LCMS (Method G): Rt=0.792 min, [M+H]⁺=549.2.

### Step 2: Synthesis of Intermediate 1-4

To a solution of Intermediate 1-3 (500 mg, 911.22 μmol, 1 eq) in DCM (5 mL) was added piperidine (2.16 g, 25.32 mmol, 2.50 mL, 27.78 eq). The mixture was stirred at 25° C. for 0.5 hr. The reaction mixture was concentrated under reduced pressure to give a residue. The residue was purified by prep-HPLC (column: Waters Xbridge 150*25 mm*5 um; mobile phase: [water (NH₄HCO₃)-ACN]; gradient: 12%-42% B over 9 min), then concentrated to remove organic solvents and lyophilized to give Intermediate 1-4 (150 mg, 459.45 μmol, 50.42% yield) as a white solid. LCMS (Method E): Rt=0.318 min, [M+H]⁺=327.3.

### Step 3: Synthesis of Intermediate 1-6

To a solution of Intermediate 1-4 (150 mg, 459.45 μmol, 1 eq) and Intermediate 1-5 (180.77 mg, 459.45 μmol, 1 eq) in DMF (1.5 mL) was added EDCI (264.23 mg, 1.38 mmol, 3 eq), HOAt (62.54 mg, 459.45 μmol, 1 eq) and NMM (464.72 mg, 4.59 mmol, 505.13 μL, 10 eq). The mixture was stirred at 25° C. for 2 hrs. The reaction mixture was quenched by addition of H₂O (2 mL), and then diluted with EA (2 mL) and extracted with EA (2 mL*3). The combined organic layers were washed with brine (2 mL*3), dried over anhydrous Na₂SO₄, filtered and concentrated under reduced pressure to give a residue. The residue was purified by reversed phase (0.1% FA condition), then concentrated to remove organic solvents and lyophilized to give Intermediate 1-6 (130 mg, 164.84 μmol, 35.88% yield, 89% purity) as a yellow solid. LCMS (Method G): Rt=0.611 min, [M+H]=702.7.

### Step 4: Synthesis of Intermediate 1-7

To a solution of Intermediate 1-6 (100 mg, 142.47 μmol, 1 eq) in DCM (0.2 mL) was added HCl/dioxane (4 M, 1 mL, 28.08 eq). The mixture was stirred at 25° C. for 1 hr. The reaction mixture was concentrated under reduced pressure to give Intermediate 1-7 (90 mg, 141.01 μmol, 98.98% yield, HCl salt) as a yellow solid. The crude product was used in next step without further purification. LCMS (Method E): Rt=0.319 min, [M+H]⁺=602.5

### Step 5: Synthesis of Intermediate 1-9

To a solution of Intermediate 1-7 (70 mg, 109.68 μmol, 1 eq, HCl salt) and Intermediate 1-8 (57.76 mg, 109.68 μmol, 1 eq) in DMF (1 mL) was added EDCI (63.07 mg, 329.03 μmol, 3 eq), HOAt (14.93 mg, 109.68 μmol, 15.34 μL, 1 eq) and NMM (110.93 mg, 1.10 mmol, 120.58 μL, 10 eq). The mixture was stirred at 25° C. for 2 hrs. The reaction mixture was quenched by addition of H₂O (1 mL), and then diluted with EA (1 mL) and extracted with EA (2 mL*3). The combined organic layers were washed with brine (2 mL*3), dried over anhydrous Na₂SO₄, filtered and concentrated under reduced pressure to give a residue. The residue was purified by reversed phase chromatography (0.1% FA condition), then concentrated to remove organic solvents and lyophilized to give Intermediate 1-9 (100 mg, 87.36 mol, 79.65% yield, 97% purity) as a white solid. LCMS (Method E): Rt=0.446 min, [M+H]⁺=1110.7.

### Step 6: Synthesis of I-148

A solution of Intermediate 1-9 (100.00 mg, 90.06 μmol, 1 eq) in added HCl (0.1 M, 3.60 mL, 4 eq) was stirred at 100° C. for 0.5 hr. The reaction mixture was basified with NH₃·H₂O and extracted with CH₃Cl:i-PrOH=3:1 (3 mL*3). The combined organic layers were dried over anhydrous Na₂SO₄, filtered and concentrated under reduced pressure to give a residue. The residue was purified by prep-HPLC (column: Waters Xbridge 150*25 mm*5 um; mobile phase: [water (NH₄HCO₃)-ACN]; gradient: 38%-68% B over 9 min), then concentrated to remove organic solvents and lyophilized to give I-148 (45.18 mg, 44.72 μmol, 49.66% yield, 100% purity) as an off-white solid. LCMS: Rt=0.391 min, [M+H]⁺=1010.6. SFC: Rt=4.582 min, ee value=100%. H NMR (400 MHz, METHANOL-d₄) δ=8.49 (d, J=2.0 Hz, 1H), 8.28 (d, J=2.8 Hz, 1H), 7.90 (d, J=8.8 Hz, 1H), 7.83 (s, 1H), 7.76 (d, J=1.2 Hz, 1H), 7.45-7.34 (m, 3H), 7.32-7.24 (m, 2H), 7.15-7.10 (m, 1H), 6.53-6.42 (m, 2H), 4.58-4.45 (m, 1H), 4.09-4.01 (m, 2H), 3.88-3.65 (m, 10H), 3.58-3.53 (m, 2H), 3.50-3.35 (m, 8H), 3.16-2.96 (m, 3H), 2.76-2.57 (m, 10H), 2.54-2.34 (m, 4H), 2.24 (d, J=6.8 Hz, 2H), 2.14-1.97 (m, 3H), 1.87-1.73 (m, 4H), 1.66-1.50 (m, 2H), 1.45-1.39 (m, 3H), 1.33-1.20 (m, 5H).

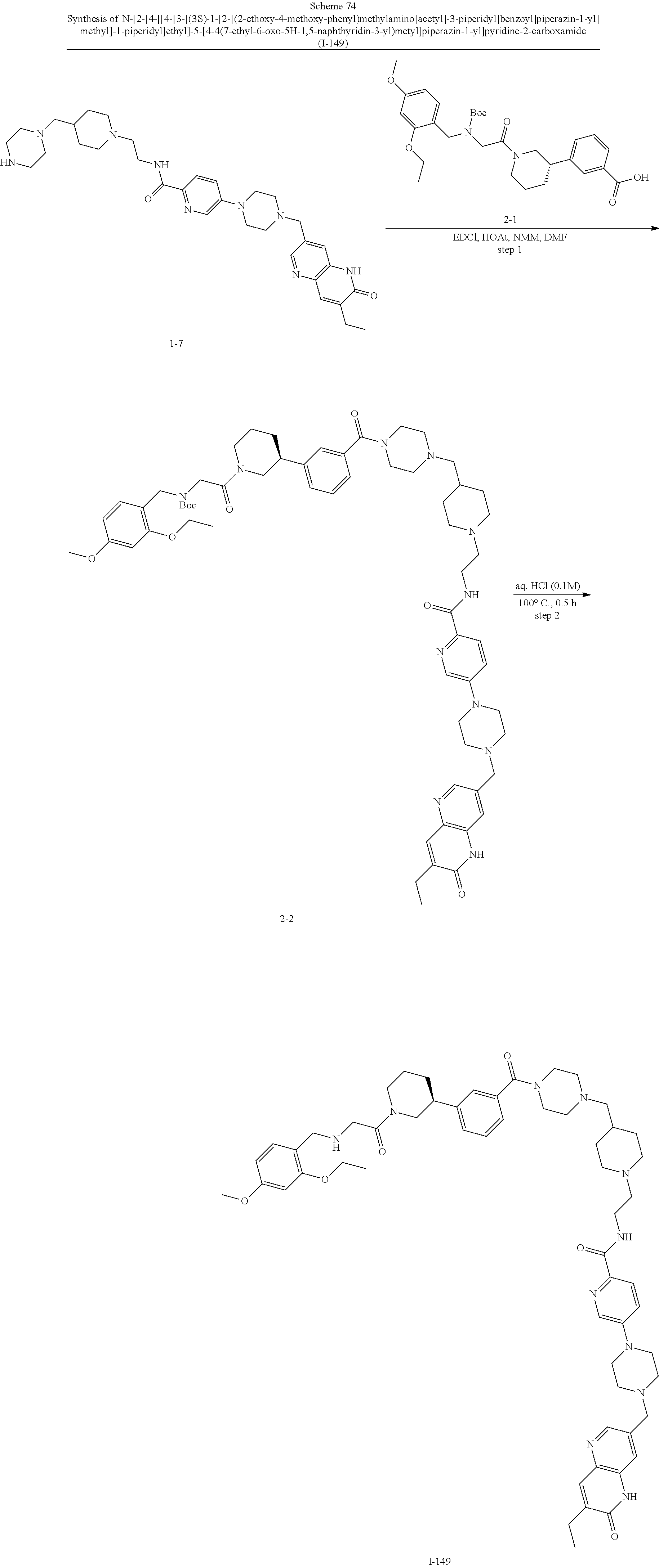 [SMILES: CCc1cc2ncc(CN3CCN(c4ccc(C(=O)NCCN5CCC(CN6CCNCC6)CC5)nc4)CC3)cc2[nH]c1=O ; CCOc1cc(OC)ccc1CN(CC(=O)N1CCC[C@@H](c2cccc(C(=O)O)c2)C1)C(=O)OC(C)(C)C ; CCOc1cc(OC)ccc1CN(CC(=O)N1CCC[C@@H](c2cccc(C(=O)N3CCN(CC4CCN(CCNC(=O)c5ccc(N6CCN(Cc7cnc8cc(CC)c(=O)[nH]c8c7)CC6)cn5)CC4)CC3)c2)C1)C(=O)OC(C)(C)C ; CCOc1cc(OC)ccc1CNCC(=O)N1CCC[C@@H](c2cccc(C(=O)N3CCN(CC4CCN(CCNC(=O)c5ccc(N6CCN(Cc7cnc8cc(CC)c(=O)[nH]c8c7)CC6)cn5)CC4)CC3)c2)C1]

### Step 1: Synthesis of Intermediate 2-2

To a solution of Intermediate 1-7 (50 mg, 78.34 μmol, 1 eq, HCl salt) and Intermediate 2-1 (41.26 mg, 78.34 μmol, 1 eq) in DMF (1 mL) was added EDCI (45.05 mg, 235.02 μmol, 3 eq), HOAt (10.66 mg, 78.34 gmol, 1 eq) and NMM (79.24 mg, 783.40 gmol, 86.13 μL, 10 eq). The mixture was stirred at 25° C. for 2 hrs. The reaction mixture was quenched by addition of H₂O (1 mL), and then diluted with EA (1 mL) and extracted with EA (2 mL*3). The combined organic layers were washed with brine (2 mL*3), dried over anhydrous Na₂SO₄, filtered and concentrated under reduced pressure to give a residue. The residue was purified by reversed phase chromatography (0.1% FA condition), then concentrated to remove organic solvents and lyophilized to give Intermediate 2-2 (70 mg, 63.04 μmol, 80.47% yield) as a yellow solid. LCMS (Method E): Rt=0.486 min [M+H]⁺=1110.9

### Step 2: Synthesis of I-149

A solution of Intermediate 2-2 (70 mg, 63.04 μmol, 1 eq) in HCl (0.1 M, 2.52 mL, 4 eq) was stirred at 100° C. for 0.5 hr. The reaction mixture was adjusted basified NH₃·H₂O and extracted with CH₃Cl:i-PrOH=3:1 (3 mL*3). The combined organic layers were dried over anhydrous Na₂SO₄, filtered and concentrated under reduced pressure to give a residue. The residue was purified by prep-HPLC (column: Phenomenex luna C18 250*50 mm*15 um; mobile phase: [water (FA)-ACN]; gradient: 2%-32% B over 9 min), then concentrated to remove organic solvents and lyophilized to give I-149 (38.72 mg, 38.33 μmol, 60.80% yield, 100% purity) as a light yellow solid. LCMS (Method E): Rt=0.384 min, [M+H]⁺=1010.9. SFC: Rt=3.467 min, ee value=95.098%. ¹H NMR (400 MHz, METHANOL-d₄) δ=8.49 (d, J=2.0 Hz, 1H), 8.29 (d, J=2.8 Hz, 1H), 7.90 (d, J=8.8 Hz, 1H), 7.83 (s, 1H), 7.76 (s, 1H), 7.46-7.34 (m, 3H), 7.33-7.25 (m, 2H), 7.18-7.14 (m Hz, 1H), 6.56-6.46 (m, 2H), 4.57-4.46 (m, 1H), 4.11-4.03 (m, 2H), 3.96-3.79 (m, 3H), 3.79-3.68 (m, 7H), 3.63-3.52 (m, 4H), 3.49-3.37 (m, 6H), 3.18-3.02 (m, 3H), 2.79-2.63 (m, 10H), 2.56-2.34 (m, 4H), 2.25 (d, J=7.2 Hz, 2H), 2.22-2.12 (m, 2H), 2.05-1.97 (m, 1H), 1.89-1.74 (m, 4H), 1.69-1.52 (m, 2H), 1.47-1.40 (m, 3H), 1.34-1.21 (m, 5H).

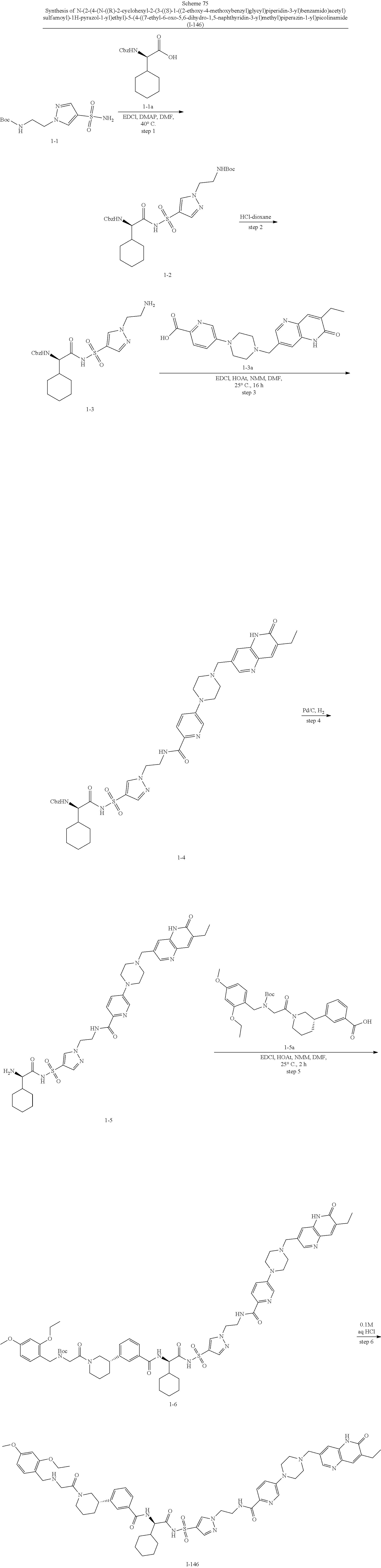

### Step 1: Synthesis of Intermediate 1-2

To a mixture of Intermediate 1-1 (200 mg, 688.85 μmol, 1 eq), Intermediate 1-1a (240.83 mg, 826.62 μmol, 1.2 eq) and DMAP (252.46 mg, 2.07 mmol, 3 eq) in DMF (2 mL) was added EDCI (660.27 mg, 3.44 mmol, 5 eq). The mixture was stirred at 40° C. for 2 hrs. The reaction was poured into MeOH (1 mL). The residue was purified by Prep-HPLC (column: Phenomenex luna C18 150*40 mm*15 um;mobile phase: [water(FA)-ACN]; gradient: 48%-78% B over 15 min) and concentrated under vacuum to remove MeCN. The aqueous phase was extracted with EA (30 mL*3), the organic phase was dried with anhydrous Na₂SO₄, filtered and concentrated under vacuum to give Intermediate 1-2 (350 mg, 620.94 μmol, 90.14% yield) as brown oil. LCMS (Method E): Rt=0.572 min, [M−99]+=464.2.

### Step 2: Synthesis of Intermediate 1-3

A mixture of Intermediate 1-2 (350 mg, 620.94 μmol, 1 eq) in DCM (3 mL) was added HCl/dioxane (2 M, 3 mL, 9.66 eq) was stirred at 25° C. for 0.5 hr. The mixture was concentrated under vacuum to give Intermediate 1-3 (0.3 g, crude, HCl salt) as yellow oil. LCMS (Method G): Rt=0.355 min, (M+H)+=464.2.

### Step 3: Synthesis of Intermediate 1-4

To a mixture of Intermediate 1-3 (0.3 g, 599.99 μmol, 1 eq, HCl salt), Intermediate 1-3a (259.66 mg, 659.99 μmol, 1.1 eq) and HOAt (81.66 mg, 599.99 μmol, 1 eq) in DMF (3 mL) was added EDCI (230.04 mg, 1.20 mmol, 2 eq) and NMM (303.44 mg, 3.00 mmol, 329.83 μL, 5 eq). The mixture was stirred at 25° C. for 3 hrs. The mixture was diluted with water (10 mL), extracted with EtOAc (10 mL*3). The organic layer was concentrated under vacuum. The residue was purified by Pre-HPLC (column: Waters Xbridge Prep OBD C18 150*40 mm*10 um; mobile phase: [water(NH4HCO3)-ACN]; gradient: 30%-60% B over 20 min) and dried by lyophilization to give Intermediate 1-4 (0.35 g, 417.18 μmol, 69.53% yield) as yellow solid. LCMS (Method E): Rt=0.465 min, (M+H)+=839.4. SFC: Rt=0.969 min.

### Step 4: Synthesis of Intermediate 1-5

To a solution of Intermediate 1-4 (350 mg, 417.18 μmol, 1 eq) in EtOH (20 mL) was added Pd(OH)₂/C (0.2 g, 20% purity). The suspension was degassed and purged with H₂ 3 times. The mixture was stirred under H₂ (15 psi) at 25° C. for 5 hr. The mixture was filtered through a pad of Celite and washed with MeOH (20 mL*5). The filtrate was concentrated under vacuum to give Intermediate 1-5 (0.3 g, crude) as an off-white oil. LCMS (Method E): Rt=0.370 min, (M+H)+=705.4.

### Step 5: Synthesis of Intermediate 1-6

To a mixture of Intermediate 1-5 (50 mg, 70.94 μmol, 1 eq), Intermediate 1-5a (29.89 mg, 56.75 mol, 0.8 eq) and HOAt (9.66 mg, 70.94 μmol, 1 eq) in DMF (1 mL) was added EDCI (27.20 mg, 141.88 mol, 2 eq) and NMM (35.88 mg, 354.69 μmol, 39.00 μL, 5 eq). The mixture was stirred at 25° C. for 1 hr. The mixture was diluted with water (2 mL), extracted with EtOAc (5 mL*3). The organic layer was concentrated under vacuum. The residue was purified by Pre-HPLC (0.1% FA condition) and dried by lyophilization to give Intermediate 1-6 (30 mg, 24.72 μmol, 34.85% yield) as white solid. LCMS (Method G): Rt=0.477 min, (M+H)+=1212.8.

### Step 6: Synthesis of I-146

A mixture of Intermediate 1-6 (30 mg, 24.72 μmol, 1 eq) in HCl (0.1 M, 0.5 mL, 2.02 eq). was stirred at 100° C. for 3 hr. The mixture was dried by lyophilization. The residue was purified by Prep-HPLC (column: Phenomenex luna C18 150*25 mm*10 um; mobile phase: [water(FA)-ACN]; gradient: 15%-45% B over 9 min) and dried by lyophilization to give I-146 (15.27 mg, 13.63 μmol, 55.13% yield, 99.371% purity) as white solid. Mass: LCMS (Method E): Rt=0.458 min, (M+H)+=1113.6. SFC: Rt=1.561 min, ¹H NMR (400 MHz, DMSO-d6) δ=12.05-11.68 (m, 1H), 8.54-8.53 (m, 1H), 8.40 (d, J=1.6 Hz, 1H), 8.26 (d, J=2.4 Hz, 1H), 7.97 (br s, 1H), 7.82 (d, J=8.8 Hz, 1H), 7.76-7.20 (m, 11H), 6.61-6.47 (m, 2H), 4.48-4.19 (m, 4H), 4.15-3.99 (m, 4H), 3.95 (br d, J=10.4 Hz, 2H), 3.89-3.80 (m, 2H), 3.75 (d, J=6.4 Hz, 3H), 3.70-3.61 (m, 6H), 2.58-2.52 (m, 7H), 1.97-1.70 (m, 5H), 1.62-1.42 (m, 6H), 1.37-1.35 (m, 2H), 1.32-1.27 (m, 2H), 1.19-1.17 (m, 3H), 1.14-0.84 (m, 7H).

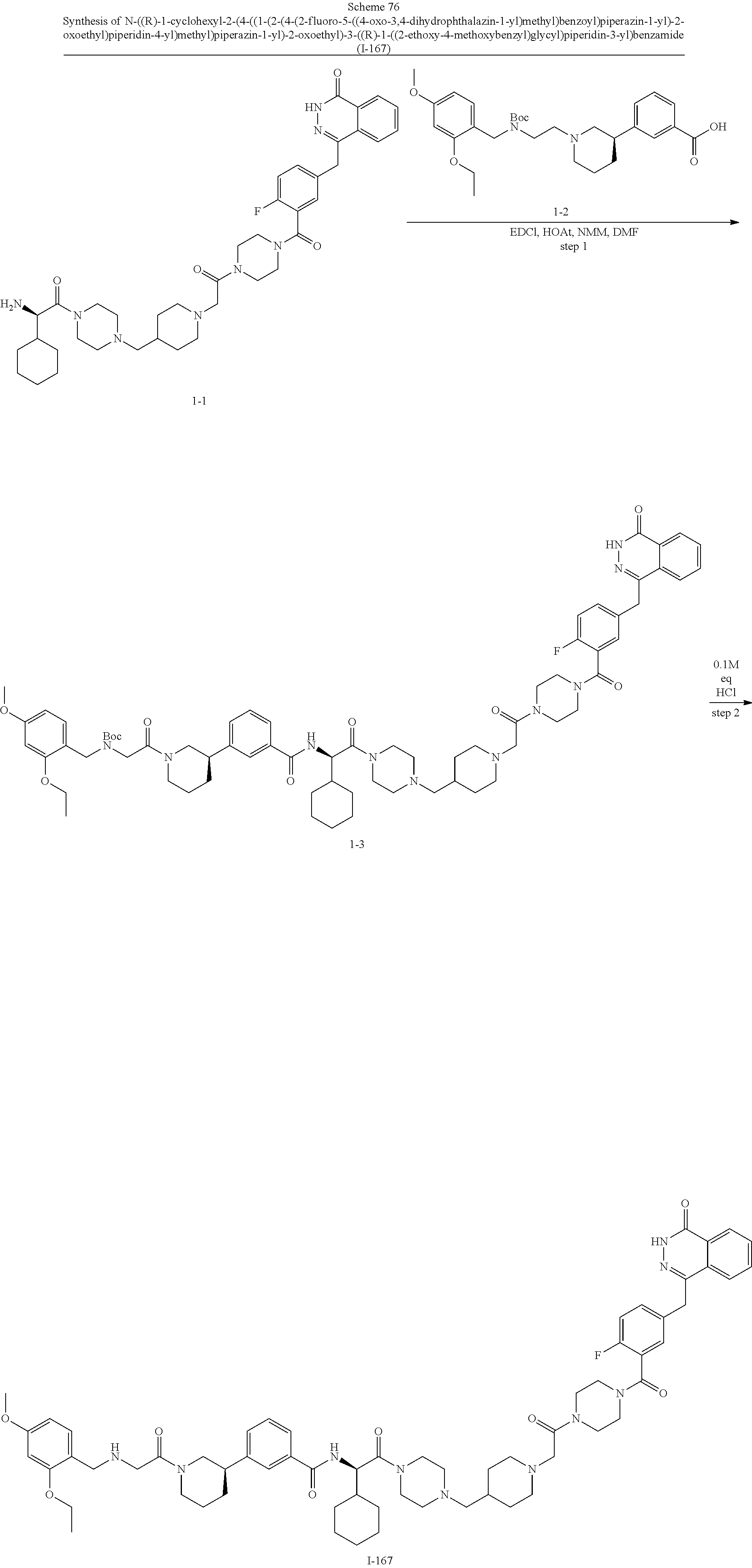

### Step 1: Synthesis of Intermediate 1-3

To a solution of Intermediate 1-1 (80 mg, 104.53 μmol, 1 eq, HCl salt) and Intermediate 1-2 (55.05 mg, 104.53 μmol, 1 eq) in DMF (1 mL) was added EDCI (60.11 mg, 313.58 μmol, 3 eq), NMM (105.73 mg, 1.05 mmol, 114.92 μL, 10 eq) and HOAt (14.23 mg, 104.53 μmol, 1 eq). The mixture was stirred at 25° C. for 1 hr. The reaction mixture was diluted with MeOH (1 mL) and purified by Prep-HPLC (FA condition), the target peak was concentrated and lyophilized to give Intermediate 1-3 (86 mg, 69.49 μmol, 66.49% yield) as a white solid, LCMS showed a major peak with desired molecular weight was found. LCMS (Method E): Rt=0.507 min, M+H=1239.1.

### Step 2: Synthesis of I-167

A solution of Intermediate 1-6 (86 mg, 69.49 μmol, 1 eq) in HCl (0.1 M, 2.08 mL, 3 eq) was stirred at 100° C. for 0.5 hr. To the mixture was added NaHCO₃ to adjust the pH to 9˜10, and then the mixture was lyophilized to give a residue. The residue was purified by Prep-HPLC (basic condition) and then lyophilized to afford I-167 (47 mg, 41.32 μmol, 59.46% yield, 100% purity) as a white solid. LCMS (Method E): Rt=0.440 min, M+H=1137.7 SFC: Rt=4.184 min, ee %=99.624%. ¹H NMR (400 MHz, CHLOROFORM-d) δ=10.45-10.01 (m, 1H), 8.53-8.27 (m, 1H), 7.80-7.65 (m, 5H), 7.43-7.31 (m, 4H), 7.20-7.13 (m, 1H), 7.11-6.93 (m, 2H), 6.47-6.39 (m, 2H), 5.11-4.64 (m, 2H), 4.29 (s, 2H), 4.07-3.97 (m, 2H), 3.86-3.57 (m, 16H), 3.55-3.23 (m, 5H), 3.23-3.12 (m, 2H), 3.08-2.94 (m, 1H), 2.89-2.79 (m, 2H), 2.73-2.54 (m, 2H), 2.49-2.36 (m, 4H), 2.20 (d, J=6.4 Hz, 2H), 2.06 (d, J=10.8 Hz, 3H), 1.88-1.80 (m, 3H), 1.74-1.50 (m, 6H), 1.46-1.39 (m, 3H), 1.34-1.01 (m, 8H).

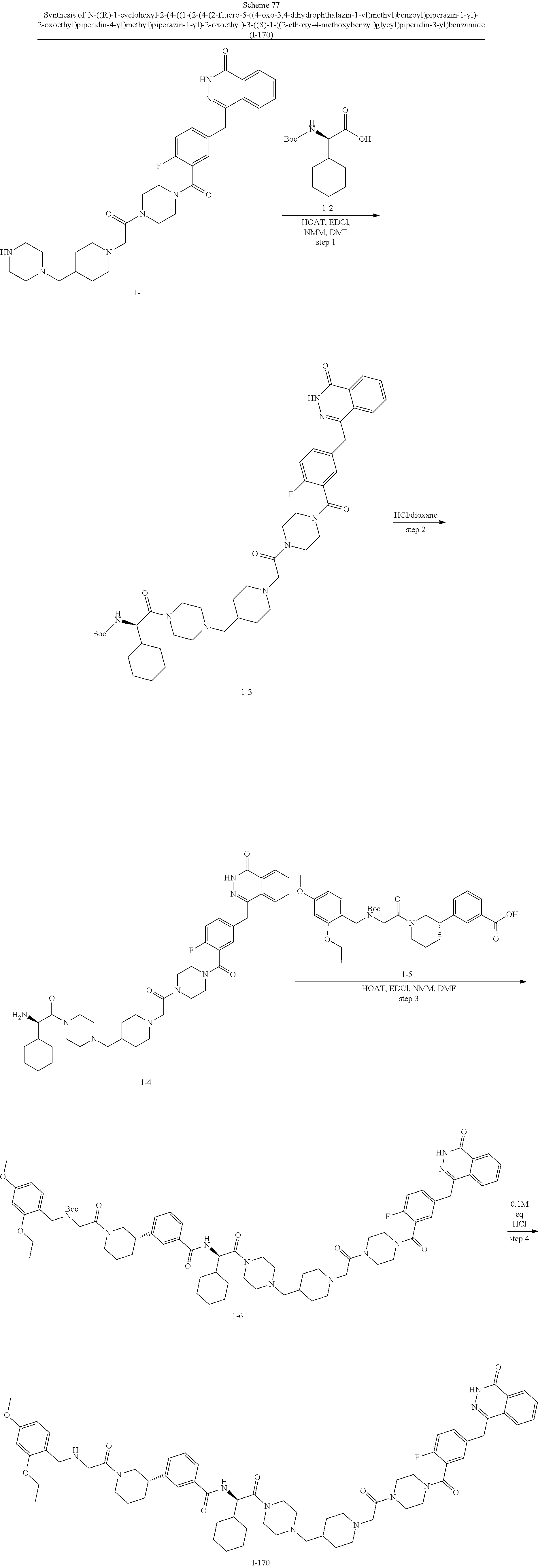

### Step 1: Synthesis of Intermediate 1-3

To a solution of Intermediate 1-1 (2.0 g, 3.19 mmol, 1 eq, HCl salt) and Intermediate 1-2 (904.10 mg, 3.51 mmol, 1.1 eq) in DMF (15 mL) was added EDCI (1.84 g, 9.58 mmol, 3 eq), NMM (3.23 g, 31.94 mmol, 3.51 mL, 10 eq) and HOAt (434.75 mg, 3.19 mmol, 1 eq). The mixture was stirred at 25° C. for 1 hr. The reaction mixture was quenched with water (10 mL). The mixture was extracted with EA (10 mL*3) and dried over anhydrous Na₂SO₄. The mixture was filtered and the filtrate was concentrated under vacuum. The residue was purified by flash silica gel chromatography (ISCO; 40 g SepaFlash® Silica Flash Column, Eluent of 0˜30% EA/MeOH @60 mL/min) to afford Intermediate 1-3 (1.56 g, 1.84 mmol, 57.74% yield, 98% purity) as a yellow solid LCMS (Method E): Rt=0.441 min, M+H=829.5

### Step 2: Synthesis of Intermediate 1-4

To a solution of Intermediate 1-3 (1.56 g, 1.88 mmol, 1 eq) in DCM (5 mL) was added HCl/dioxane (10 mL). The mixture was stirred at 25° C. for 0.5 hr. The reaction mixture was concentrated under the pressure to give Intermediate 1-4 (2.2 g, crude, HCl salt) as a white solid. LCMS (Method E): Rt=0.380 min, M+H=729.6 SFC: Rt=0.721 min

### Step 3: Synthesis of Intermediate 1-6

To a solution of Intermediate 1-4 (80 mg, 109.75 μmol, 1 eq) and Intermediate 1-5 (57.80 mg, 109.75 μmol, 1 eq) in DMF (1 mL) was added EDCI (63.12 mg, 329.26 μmol, 3 eq), NMM (111.01 mg, 1.10 mmol, 120.67 μL, 10 eq) and HOAt (14.94 mg, 109.75 μmol, 15.35 μL, 1 eq). The mixture was stirred at 25° C. for 1 hr. The reaction mixture was diluted with MeOH (1 mL) and purified by Prep-HPLC (FA condition), the target peak was concentrated and lyophilized to give Intermediate 1-6 (80 mg, 64.65 μmol, 58.90% yield) as a white solid. LCMS: Rt=0.508 min, M+H=1238.2 LCMS: Rt=0.508 min, M+H=1238.0

### Step 4: Synthesis of I-170

A solution of Intermediate 1-6 (80 mg, 64.65 μmol, 1 eq) in HCl (0.1 M, 1.94 mL, 3 eq) was stirred at 100° C. for 0.5 hr. To the mixture was added NaHCO₃ to adjust the pH to 9˜10, and then the mixture was lyophilized to give a residue. The residue was purified by Prep-HPLC (basic condition) and then lyophilized to afford I-170 (41 mg, 36.05 μmol, 55.76% yield, 100% purity) as a white solid. LCMS (Method E): Rt=0.445 min, M+H=1138.7 SFC: Rt=3.526 min, ee %=95.31%. ¹H NMR (400 MHz, CHLOROFORM-d) δ 10.32-9.91 (m, 1H), 8.56-8.40 (m, 1H), 7.83-7.61 (m, 5H), 7.42-7.31 (m, 4H), 7.22-7.13 (m, 1H), 7.11-7.00 (m, 2H), 6.47-6.40 (m, 2H), 5.05 (s, 1H), 4.70 (d, J=11.2 Hz, 1H), 4.29 (s, 2H), 4.07-3.97 (m, 2H), 3.90-3.56 (m, 16H), 3.56-3.24 (m, 5H), 3.22-3.13 (m, 2H), 3.09-2.94 (m, 1H), 2.92-2.78 (m, 2H), 2.76-2.53 (m, 2H), 2.50-2.33 (m, 4H), 2.20 (d, J=6.4 Hz, 2H), 2.10-2.01 (m, 3H), 1.84 (d, J=2.8 Hz, 3H), 1.71-1.49 (m, 6H), 1.46-1.39 (m, 3H), 1.33-1.05 (m, 8H).

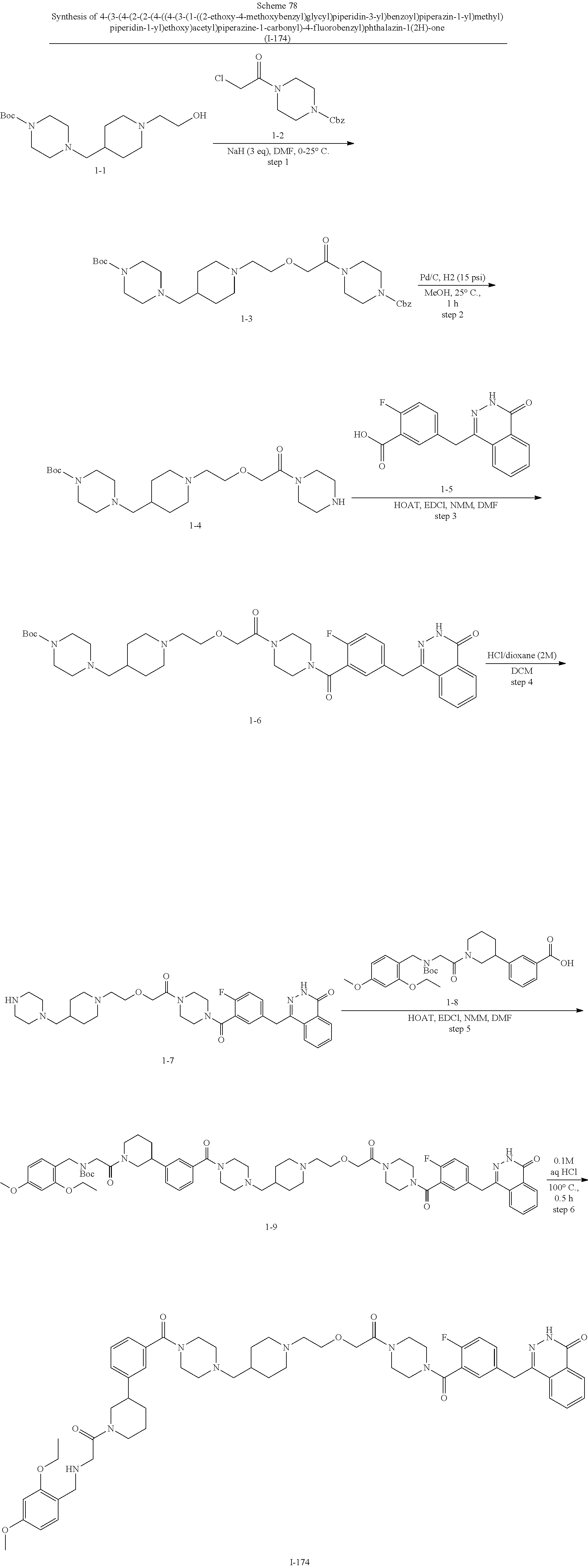

### Step 1: Synthesis of Intermediate 1-3

To a solution of Intermediate 1-1 (500 mg, 1.53 mmol, 1 eq) in DMF (5 mL) was added NaH (183.21 mg, 4.58 mmol, 60% purity, 3 eq) at 0° C. for 0.5 h. Then Intermediate 1-2 (906.21 mg, 3.05 mmol, 2 eq) was added and the mixture was stirred at 25° C. for 1.5 hr. The reaction mixture was concentrated under reduced pressure to give the crude product. The crude product was quenched with a saturated NH₄Cl (15 mL) solution and extracted with EA (10 mL*3). The combined organic phase was dried over anhydrous Na₂SO₄, filtered and concentrated to give Intermediate 1-3 (880 mg, 1.48 mmol, 97.23% yield, 99.16% purity) as a white solid. LCMS (Method E): Rt=0.393 min, [M+H]⁺=588.5.

### Step 2: Synthesis of Intermediate 1-4

To a solution of Intermediate 1-3 (300 mg, 510.42 μmol, 1 eq) in MeOH (2 mL) was added Pd/C (50 mg, 10% purity), and the suspension was degassed under H₂ (15 psi) and purged with H₂ (15 psi) three times. The mixture was stirred at 25° C. for 1 hr. The mixture was filtered and washed with MeOH (2 mL*3) to give the filtrate. The filtrate was concentrated to give Intermediate 1-4 (230 mg, 456.33 μmol, 89.40% yield, 90% purity) as yellow oil. LCMS (Method E): Rt=0.318 min, [M+H]⁺=454.3.

### Step 3: Synthesis of Intermediate 1-6

To a solution of Intermediate 1-5 (166.36 mg, 557.74 μmol, 1.1 eq) and Intermediate 1-4 (230 mg, 507.04 μmol, 1 eq) in DMF (0.3 mL) was added EDCI (291.60 mg, 1.52 mmol, 3 eq), HOAt (69.01 mg, 507.04 μmol, 1 eq) and NMM (512.87 mg, 5.07 mmol, 557.46 μL, 10 eq). The mixture was stirred at 25° C. for 1 hr. The reaction mixture was concentrated under reduced pressure to give the crude product. The crude product was purified by reversed phased HPLC (FA condition), and the target peak was concentrated and extracted with EA (10 mL*2), dried over anhydrous sodium sulfate, filtered and concentrated to give Intermediate 1-6 (360 mg, 482.21 μmol, 95.10% yield, 98.30% purity) as a white solid. LCMS (Method E): Rt=0.382 min, [M+H]⁺=734.4.

### Step 4: Synthesis of Intermediate 1-7

To a solution of Intermediate 1-6 (300 mg, 408.79 μmol, 1 eq) in DCM (2 mL) was added HCl/dioxane (2 M, 3 mL, 14.68 eq). The mixture was stirred at 25° C. for 1 hr. The reaction mixture was concentrated under reduced pressure to give Intermediate 1-7 (270 mg, 330.34 μmol, 80.81% yield, 82% purity, HCl salt) as a yellow solid. LCMS (Method E): Rt=0.358 min, [M+H]⁺=634.3

### Step 5: Synthesis of Intermediate 1-9

To a solution of Intermediate 1-7 (150 mg, 223.81 μmol, 1 eq, HCl salt) and Intermediate 1-8 (153.22 mg, 290.95 μmol, 1.3 eq) in DMF (3 mL) was added EDCI (128.71 mg, 671.43 μmol, 3 eq), HOAt (30.46 mg, 223.81 μmol, 1 eq) and NMM (226.38 mg, 2.24 mmol, 246.06 μL, 10 eq). The mixture was stirred at 25° C. for 1 hr. The mixture was poured into water (10 mL) and extracted with EA (10 mL*3). The combined organic layers were concentrated to give a residue. The residue was purified by reversed phased HPLC (FA), the target peak was concentrated and then extracted with EA (20 mL*2), dried over anhydrous sodium sulfate, filtered and concentrated to give Intermediate 1-9 (150 mg, 129.99 μmol, 58.08% yield, 99% purity) as a yellow solid. LCMS (Method E): Rt=0.482 min, [M+H]⁺=1142.6.

### Step 6: Synthesis of I-174

A solution of Intermediate 1-9 (120 mg, 105.05 μmol, 1 eq) in HCl (0.1 M, 4.20 mL, 4 eq) was stirred at 100° C. for 0.5 hr. The reaction mixture was concentrated under reduced pressure to give the crude product. The crude product was purified by prep-HPLC (column: YMC-Actus Triart C18 150*30 mm*7 um; mobile phase: [water (FA)-ACN]; gradient: 10%-40% B over 10 min) and the eluent was concentrated to remove MeCN and then lyophilized to afford I-174 (61.76 mg, 56.18 μmol, 53.48% yield, 99% purity, FA salt) as a brown solid. LCMS (Method E): Rt=0.405 min, [M+H]⁺=1042.7. SFC: Rt=5.587 min, 6.440 min, ¹H NMR (400 MHz, METHANOL-d₄) δ 8.44 (s, 2H), 8.37 (d, J=7.6 Hz, 1H), 7.98-7.92 (m, 1H), 7.91-7.81 (m, 2H), 7.51 (s, 1H), 7.47-7.39 (m, 2H), 7.38-7.25 (m, 4H), 7.17-7.10 (m, 1H), 6.65-6.54 (m, 2H), 4.57-4.48 (m, 1H), 4.46-4.34 (m, 4H), 4.23-4.10 (m, 4H), 4.07-3.93 (m, 2H), 3.90-3.40 (m, 18H), 3.39-3.32 (m, 4H), 3.17-3.10 (m, 1H), 3.06 (d, J=10.4 Hz, 2H), 2.86-2.67 (m, 2H), 2.61-2.38 (m, 4H), 2.33 (s, 2H), 2.06-2.00 (m, 3H), 1.98-1.77 (m, 3H), 1.70-1.42 (m, 6H).

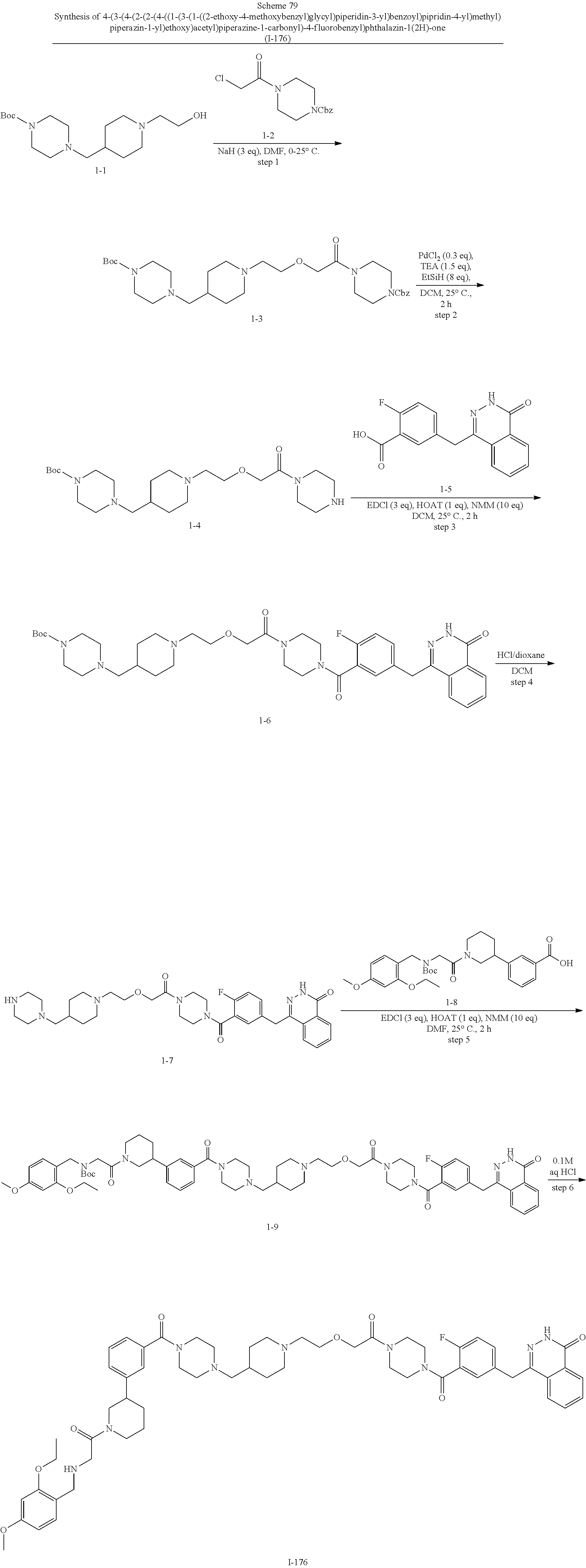

### Step 1: Synthesis of Intermediate 1-3

To a solution of Intermediate 1-1 (1 g, 3.05 mmol, 1 eq) in DMF (1 mL) was added NaH (366.42 mg, 9.16 mmol, 60% purity, 3 eq). The mixture was stirred at 0° C. for 0.5 h, then Intermediate 1-2 (1.81 g, 6.11 mmol, 2 eq) was added to the mixture, and the mixture was stirred at 25° C. for 1.5 h. The reaction mixture was quenched with a saturated NH₄Cl solution and extracted with EA (20 mL*3). The combined organic phase was dried over anhydrous Na₂SO₄, filtered and concentrated to give a residue. The residue was purified by reversed phase chromatography (FA condition), then concentrated to remove organic solvents and lyophilized to give Intermediate 1-3 (1.3 g, 707.79 μmol, 23.18% yield, 32% purity) as a yellow oil LCMS (Method E): Rt=0.448 min, [M+H]⁺=588.4.

### Step 2: Synthesis of Intermediate 1-4

To a solution of Intermediate 1-3 (300 mg, 510.42 μmol, 1 eq) in DCM (3 mL) was added PdCl₂ (27.15 mg, 153.13 μmol, 0.3 eq), TEA (77.47 mg, 765.63 μmol, 106.57 μL, 1.5 eq) and Et₃SiH (474.80 mg, 4.08 mmol, 652.20 μL, 8 eq). The mixture was stirred at 25° C. for 2 hr. The reaction mixture was concentrated under reduced pressure to give a residue. The residue was purified by reversed phase chromatography (NH₃·H₂O condition), then concentrated to remove organic solvents and lyophilized to give Intermediate 1-4 (100 mg, 220.45 μmol, 43.19% yield, 100% purity) as colorless oil. LCMS (Method E): Rt=0.3518 min, [M+H]⁺=454.4.

### Step 3: Synthesis of Intermediate 1-6

To a solution of Intermediate 1-4 (100 mg, 220.45 μmol, 1 eq) and Intermediate 1-5 (72.33 mg, 242.50 μmol, 1.1 eq) in DCM (1 mL) was added EDCI (126.78 mg, 661.35 μmol, 3 eq), HOAt (30.01 mg, 220.45 μmol, 1 eq) and NMM (222.98 mg, 2.20 mmol, 242.37 μL, 10 eq). The mixture was stirred at 25° C. for 2 hr. The reaction mixture was concentrated under reduced pressure to give a residue. The residue was purified by reversed phase ( ), then concentrated to remove organic solvents and lyophilized to give Intermediate 1-6 (110 mg, 148.39 μmol, 67.31% yield, 99% purity) as a white solid. LCMS (Method G): Rt=0.608 min, [M+H]⁺=734.4.

### Step 4: Synthesis of Intermediate 1-7

A solution of Intermediate 1-6 (310 mg, 422.42 μmol, 1 eq) in DCM (2 mL) in HCl/dioxane (2 M, 3.10 mL, 14.68 eq) was stirred at 25° C. for 1 hr. The reaction mixture was concentrated under reduced pressure to give a residue. The residue was used in next step without further purification. Intermediate 1-7 (240 mg, crude) was obtained as a white solid. LCMS (Method E): Rt=0.362 min, [M+H]⁺=634.4.

### Step 5: Synthesis of Intermediate 1-9

To a solution of Intermediate 1-7 (100 mg, 157.79 μmol, 1 eq) and Intermediate 1-8 (83.10 mg, 157.79 μmol, 1 eq) in DMF (1 mL) was added EDCI (90.75 mg, 473.37 μmol, 3 eq), HOAt (21.48 mg, 157.79 μmol, 1 eq) and NMM (159.60 mg, 1.58 mmol, 173.48 μL, 10 eq). The mixture was stirred at 25° C. for 2 hr. The reaction mixture was quenched by addition of H₂O (1 mL), and then diluted with EA (1 mL) and extracted with EA (2 mL*3). The combined organic layers were washed with brine (2 mL*3), dried over anhydrous Na₂SO₄, filtered and concentrated under reduced pressure to give a residue. The residue was purified by reversed phase chromatography (FA condition), then concentrated to remove organic solvents and lyophilized to give Intermediate 1-9 (105 mg, 91.92 μmol, 58.25% yield, 100% purity) as a white solid. LCMS (Method E): Rt=0.497 min, [M+H]⁺=1142.6.

### Step 6: Synthesis of I-176

A solution of Intermediate 1-9 (85 mg, 74.41 μmol, 1 eq) in HCl (0.1 M, 2.98 mL, 4 eq). The mixture was stirred at 100° C. for 0.5 hr. The reaction mixture was basified with NH₃·H₂O and extracted with CH₃Cl: i-PrOH (3:1) (3 mL*3). The combined organic layers were dried over anhydrous Na₂SO₄, filtered and concentrated under reduced pressure to give a residue. The residue was purified by prep-HPLC (column: YMC-Actus Triart C18 150*30 mm*7 um; mobile phase: [water (FA)-ACN]; gradient: 13%-43% B over 10 min), then concentrated to remove organic solvents and lyophilized to give I-176 (41.2 mg, 37.86 μmol, 50.88% yield, 100% purity, FA) as a white solid. LCMS (Method E): Rt=0.406 min, [M+H]⁺=1042.9. SFC: Rt=4.653 min, 5.407 min. ¹H NMR (400 MHz, METHANOL-d₄) δ=8.54 (s, 1H), 8.37-8.35 (m, 1H), 7.93 (d, J=3.6 Hz, 1H), 7.90-7.79 (m, 2H), 7.51-7.33 (m, 4H), 7.31-7.23 (m, 2H), 7.21-7.12 (m, 2H), 6.56-6.46 (m, 2H), 4.67-4.47 (m, 3H), 4.37 (s, 2H), 4.28-4.18 (m, 2H), 4.12-4.04 (m, 2H), 3.93-3.86 (m, 2H), 3.82-3.62 (m, 12H), 3.62-3.54 (m, 2H), 3.54-3.43 (m, 2H), 3.19-3.03 (m, 2H), 2.90-2.81 (m, 1H), 2.78-2.58 (m, 8H), 2.57-2.36 (m, 4H), 2.29-2.16 (m, 2H), 2.01 (d, J=11.2 Hz, 1H), 1.92-1.68 (m, 5H), 1.66-1.50 (m, 1H), 1.48-1.38 (m, 3H), 1.27-1.05 (m, 2H).

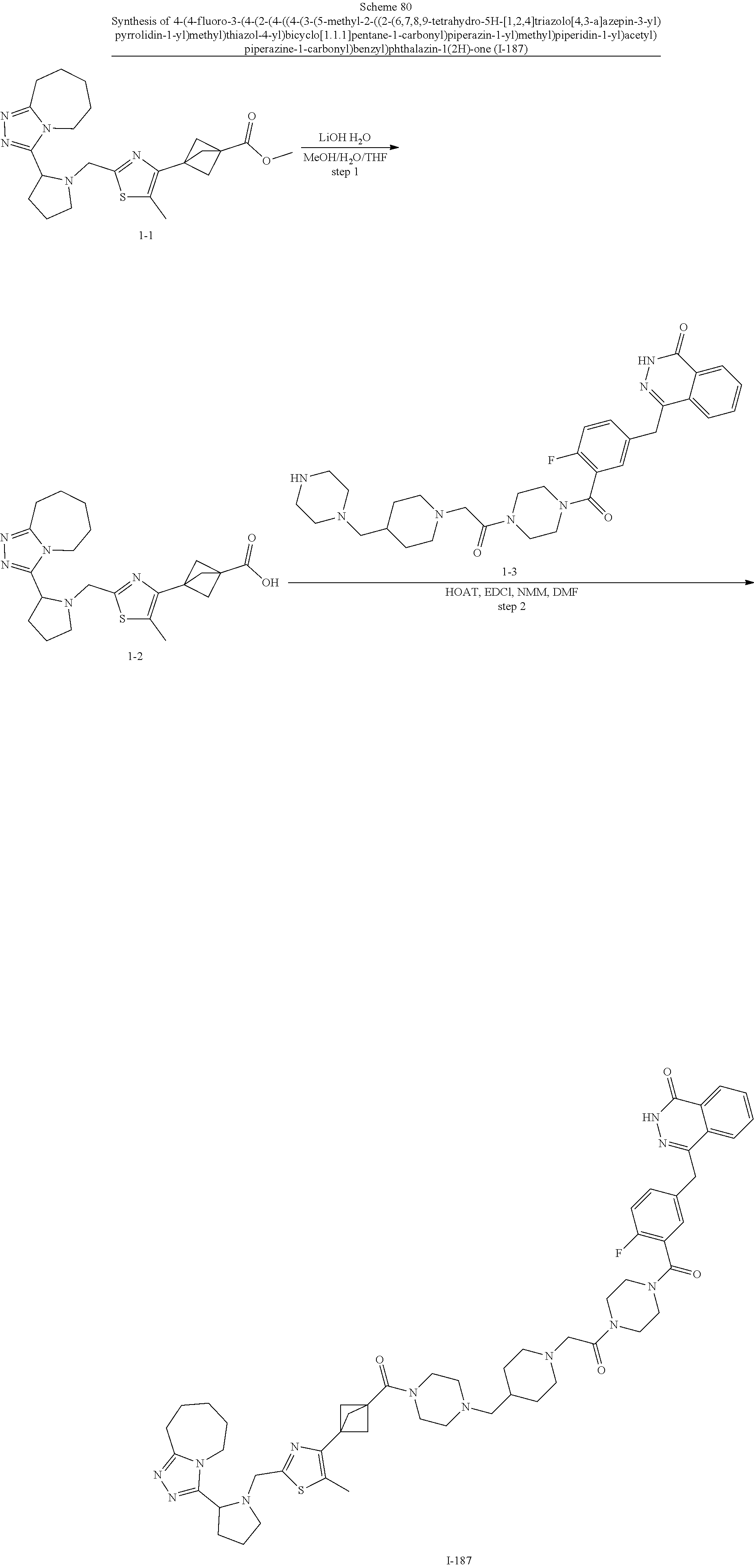 [SMILES: COC(=O)C12CC(c3nc(CN4CCCC4c4nnc5n4CCCCC5)sc3C)(C1)C2 ; "LiOH H2O" ; Cc1sc(CN2CCCC2c2nnc3n2CCCCC3)nc1C12CC(C(=O)O)(C1)C2 ; O=C(CN1CCC(CN2CCNCC2)CC1)N1CCN(C(=O)c2cc(Cc3n[nH]c(=O)c4ccccc34)ccc2F)CC1 ; Cc1sc(CN2CCCC2c2nnc3n2CCCCC3)nc1C12CC(C(=O)N3CCN(CC4CCN(CC(=O)N5CCN(C(=O)c6cc(Cc7n[nH]c(=O)c8ccccc78)ccc6F)CC5)CC4)CC3)(C1)C2]

### Step 1: Synthesis of Intermediate 1-3

To a solution of Intermediate 1-1 (0.5 g, 1.13 mmol, 1 eq) in MeOH (1.25 mL), H₂O (1.25 mL) and THF (2.5 mL) was added LiOH H₂O (95.03 mg, 2.26 mmol, 2 eq). The mixture was stirred at 25° C. for 1 hrs. The mixture was adjusted to pH=4 with 2M HCl and concentrated under vacuum. The crude product was purified by reversed-phase HPLC (0.1% FA condition) and concentrated under vacuum to remove MeCN and dried by lyophilization to give Intermediate 1-2 (295 mg, 689.96 μmol, 60.94% yield) was obtained as white solid. LCMS: Rt=0.408 min, (M+H)+=428.2.

### Step 2: Synthesis of I-187

A mixture of Intermediate 1-2 (30 mg, 70.17 μmol, 1 eq), Intermediate 1-3 (54.44 mg, 70.17 mol, 1 eq) and HOAt (9.55 mg, 70.17 μmol, 9.82 μL, 1 eq) in DMF (0.5 mL) was added EDCI (26.90 mg, 140.33 μmol, 2 eq) and NMM (35.49 mg, 350.83 μmol, 38.57 μL, 5 eq). The mixture was stirred at 25° C. for 1.5 hrs. The mixture was poured into MeOH (1 mL). The residue was purified by Pre-HPLC (column: Phenomenex luna C18 150*25 mm*10 um; mobile phase: [water(FA)-ACN]; gradient: 8%-38% B over 9 min) and dried by lyophilization to give I-187 (38.51 mg, 37.93 μmol, 54.05% yield, 98.412% purity) as white solid. LCMS (Method G): Rt=1.658 min, (M+H)+=999.8. SFC: Rt=1.321 min, 3.083 min. ¹H NMR (400 MHz, DMSO-d₆) δ=12.59 (s, 1H), 8.26 (d, J=7.6 Hz, 1H), 8.16 (s, 1H), 8.00-7.93 (m, 1H), 7.89-7.88 (m, 1H), 7.86-7.80 (m, 1H), 7.44 (br s, 1H), 7.37-7.36 (m, 1H), 7.25-7.23 (m, 1H), 4.33 (s, 2H), 4.29-4.12 (m, 2H), 3.95-3.94 (m, 1H), 3.74 (d, J=14.8 Hz, 1H), 3.65-3.48 (m, 10H), 3.19-3.07 (m, 6H), 2.87-2.72 (m, 4H), 2.36 (d, J=6.0 Hz, 9H), 2.33-2.21 (m, 5H), 2.19-2.06 (m, 3H), 1.99-1.83 (m, 5H), 1.78 (br d, J=5.6 Hz, 2H), 1.70-1.51 (m, 6H), 1.49-1.38 (m, 1H), 1.17-0.95 (m, 2H). ¹⁹F NMR (377 MHz, DMSO-d₆): δ=−119.8.

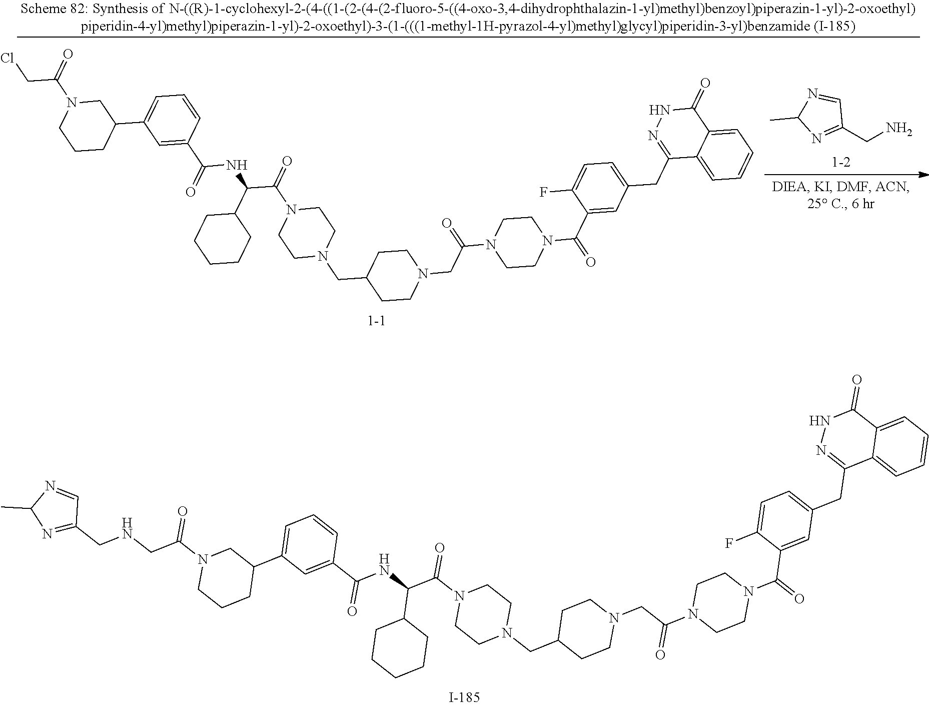

To a solution of Intermediate 1-2 (8.40 mg, 75.56 μmol, 1.5 eq) in ACN (0.5 mL) and DMF (0.5 mL) was added DIEA (26.04 mg, 201.49 μmol, 35.10 μL, 4 eq) under N₂ and it was stirred at 25° C. for 0.5 h. Then KI (4.18 mg, 25.19 μmol, 0.5 eq) and Intermediate 1-1 (50 mg, 50.37 μmol, 1 eq) were added into the mixture and it was stirred at 25° C. for 5.5 hrs. The reaction mixture was quenched with water (5 mL) at 25° C., and then extracted with EA (5 mL*3). Then the organic phase was dried over Na₂SO₄, filtered and concentrated under reduced pressure to give a residue. The residue was purified by prep-HPLC (column: Phenomenex luna C18 150*25 mm*10 um; mobile phase: [water (FA)-ACN]; gradient: 5%-35% B over 9 min). The major peak was concentrated and lyophilized to afford I-185 (20.05 mg, 18.37 μmol, 36.48% yield, 97.811% purity) as a white solid. LCMS (Method E): Rt=0.405 min, [M+H]⁺=1067.7. SFC: Rt=4.394 min, 4.804 min. ¹H NMR (400 MHz, METHANOL-d₄) δ=8.38-8.36 (d, J=7.2 Hz, 1H), 7.99-7.92 (m, 1H), 7.91-7.81 (m, 2H), 7.79-7.69 (m, 3H), 7.60-7.57 (d, J=9.2 Hz, 1H), 7.53-7.43 (m, 3H), 7.38 (m, 1H), 7.17 (m, 1H), 4.94-4.90 (m, 1H), 4.62-4.46 (m, 1H), 4.39 (s, 2H), 4.11-4.06 (m, 2H), 4.01 (m, 1H), 3.91-3.89 (d, J=8.4 Hz, 3H), 3.83-3.67 (m, 9H), 3.66-3.51 (m, 4H), 3.48 (s, 1H), 3.36 (s, 1H), 3.28-3.18 (m, 3H), 2.91-2.71 (m, 2H), 2.63-2.61 (d, J=9.2 Hz, 2H), 2.52 (s, 2H), 2.45-2.44 (d, J=2.8 Hz, 2H), 2.27 (m, 2H), 2.12-2.00 (m, 1H), 1.89 (s, 6H), 1.81-1.65 (m, 6H), 1.45-1.23 (m, 5H), 1.21-1.03 (m, 3H). ¹⁹F NMR (377 MHz, METHANOL-d₄) δ=−120.68.

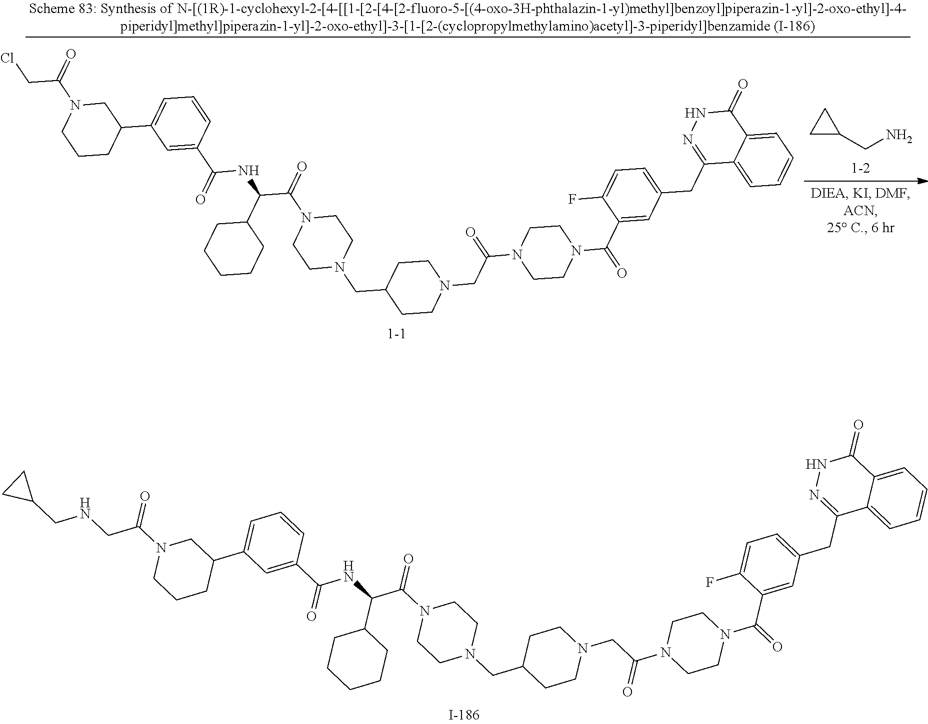

To a solution of Intermediate 1-2 (7.16 mg, 100.74 μmol, 2 eq) in ACN (0.5 mL) and DMF (0.5 mL) was added DIEA (26.04 mg, 201.49 μmol, 35.10 μL, 4 eq) under N₂, and the reaction mixture was stirred at 25° C. for 0.5 h. Then KI (4.18 mg, 25.19 μmol, 0.5 eq) and Intermediate 1-1 (50 mg, 50.37 mol, 1 eq) were added into the mixture, and the reaction mixture was stirred at 25° C. for 5.5 hrs. The reaction mixture was quenched with water (5 mL) at 25° C., and then extracted with EA (5 mL*3). The combined organic layers were dried over Na₂SO₄, filtered and concentrated under reduced pressure to give a residue. The residue was purified by prep-HPLC (column: Phenomenex luna C18 150*25 mm*10 um; mobile phase: [water (FA)-ACN]; gradient: 5%-35% B over 9 min), and the major peak was concentrated and lyophilized to afford I-186 (22.35 mg, 19.58 μmol, 38.88% yield, 100% purity, TFA salt) as a white solid. LCMS (Method E): Rt=0.416 min, [M+H]⁺=1027.7. SFC: Rt=3.601 min, 4.265 min. ¹H NMR (400 MHz, METHANOL-d₄) δ=8.38-8.36 (d, J=7.6 Hz, 1H), 8.00-7.93 (m, 1H), 7.92-7.81 (m, 2H), 7.81-7.69 (m, 2H), 7.56-7.42 (m, 3H), 7.39 (m, 1H), 7.17 (m, 1H), 4.93-4.90 (d, J=9.6 Hz, 1H), 4.62-4.46 (m, 1H), 4.39 (s, 2H), 4.22-3.98 (m, 2H), 3.88-3.67 (m, 9H), 3.67-3.45 (m, 5H), 3.37 (s, 1H), 3.28-3.19 (m, 3H), 2.97-2.74 (m, 4H), 2.64-2.39 (m, 6H), 2.27 (m, 2H), 2.09-2.06 (d, J=11.2 Hz, 1H), 1.90 (m, 6H), 1.82-1.65 (m, 6H), 1.45-1.23 (m, 5H), 1.18-1.09 (m, 3H), 0.72 (m, 2H), 0.47-0.37 (m, 2H). ¹⁹F NMR (377 MHz, METHANOL-d₄), 6=−76.87, −120.76.

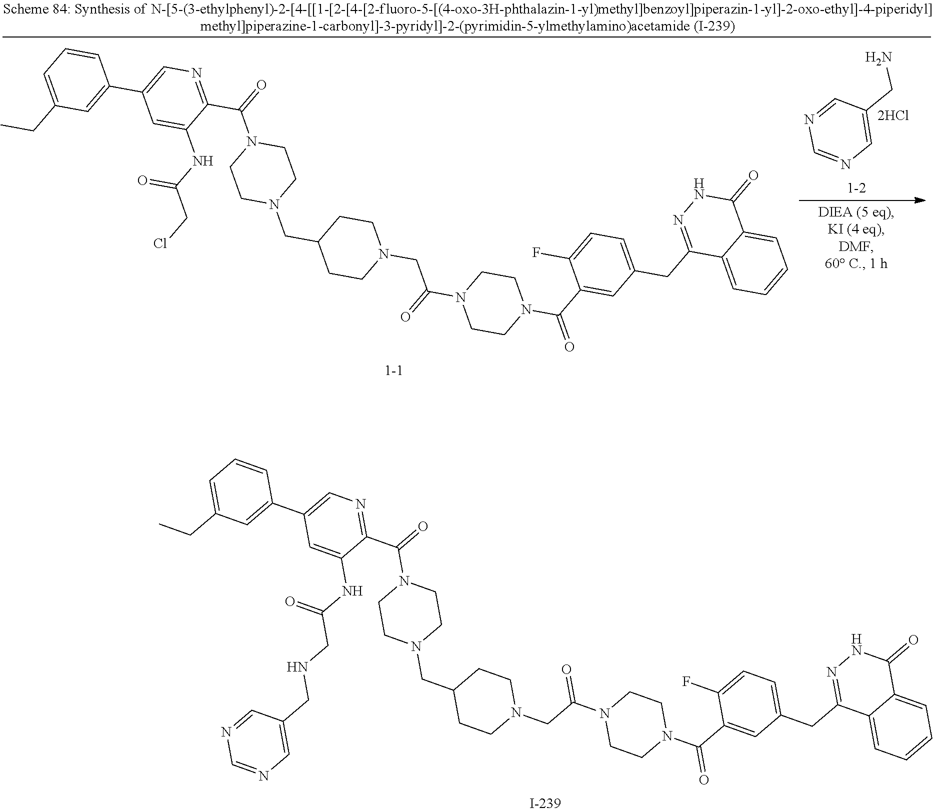 [SMILES: CCc1cccc(-c2cnc(C(=O)N3CCN(CC4CCN(CC(=O)N5CCN(C(=O)c6cc(Cc7n[nH]c(=O)c8ccccc78)ccc6F)CC5)CC4)CC3)c(NC(=O)CCl)c2)c1 ; NCc1cncnc1 ; CCc1cccc(-c2cnc(C(=O)N3CCN(CC4CCN(CC(=O)N5CCN(C(=O)c6cc(Cc7n[nH]c(=O)c8ccccc78)ccc6F)CC5)CC4)CC3)c(NC(=O)CNCc3cncnc3)c2)c1]

To a solution of Intermediate 1-1 (50 mg, 56.15 μmol, 1 eq) and Intermediate 1-2 (12.26 mg, 84.23 μmol, 1.5 eq, HCl salt) in DMF (0.5 mL) was added DIEA (36.29 mg, 280.76 gmol, 48.90 μL, 5 eq) and KI (37.29 mg, 224.61 μmol, 4 eq). The mixture was stirred at 60° C. for 1 hr. The reaction mixture was washed with water and extracted with EA 3 times, and the separated organic phase was concentrated to give the crude product. The crude product was purified by prep-HPLC (column: Phenomenex luna C18 150*25 mm*10 um; mobile phase: [water (FA)-ACN]; gradient: 2%-32% B over 15 min), then concentrated to remove organic solvents and lyophilized to give I-239 (17.3 mg, 17.96 gmol, 31.99% yield, 100% purity) as a white solid. LCMS (Method E): Rt=0.408 min [M+H]⁺=963.5. ¹H NMR (400 MHz, METHANOL-d₄) 6=9.07 (s, 1H), 8.98 (s, 2H), 8.88 (d, J=2.0 Hz, 1H), 8.60 (d, J=1.6 Hz, 1H), 8.36 (d, J=7.6 Hz, 1H), 8.00-7.92 (m, 1H), 7.91-7.80 (m, 2H), 7.55-7.46 (m, 3H), 7.46-7.29 (m, 3H), 7.19-7.14 (m, 1H), 4.65-4.57 (m, 1H), 4.38 (s, 2H), 3.91 (s, 2H), 3.85-3.63 (m, 6H), 3.58 (s, 2H), 3.55-3.45 (m, 5H), 3.38-3.33 (m, 1H), 3.08 (d, J=6.4 Hz, 2H), 2.77-2.71 (m, 2H), 2.56-2.17 (m, 8H), 1.91-1.77 (m, 2H), 1.72-1.58 (m, 1H), 1.41-1.23 (m, 6H). ¹⁹F NMR (377 MHz, METHANOL-d₄), δ=−120.75.

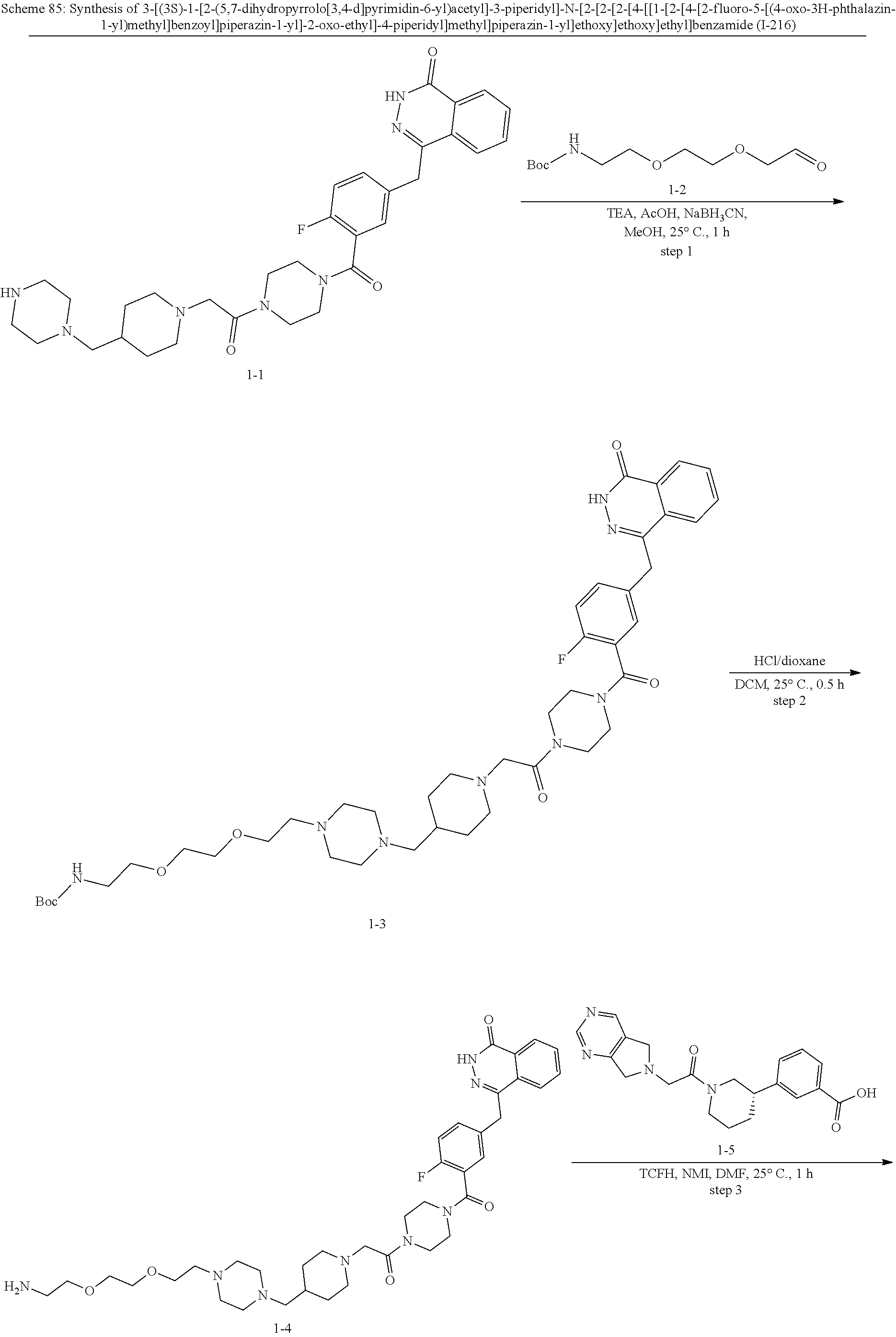

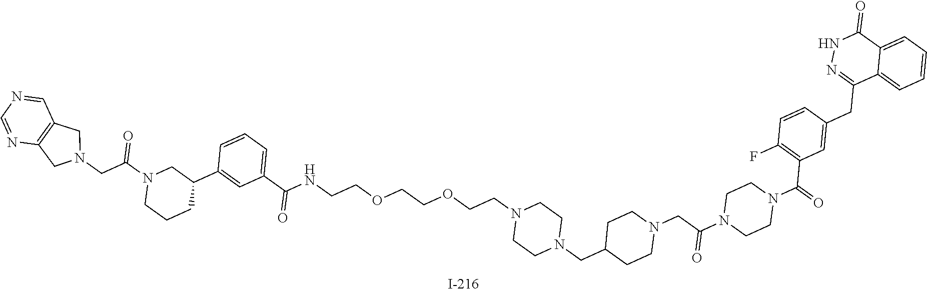 [SMILES: O=C(NCCOCCOCCN1CCN(CC2CCN(CC(=O)N3CCN(C(=O)c4cc(Cc5n[nH]c(=O)c6ccccc56)ccc4F)CC3)CC2)CC1)c1cccc([C@@H]2CCCN(C(=O)CN3Cc4cncnc4C3)C2)c1]

### Step 1: Synthesis of Intermediate 1-3

To a mixture of intermediate 1-1 (500 mg, 798.51 μmol, 1 eq, HCl salt) in MeOH (5 mL) was added TEA (242.40 mg, 2.40 mmol, 333.43 μL, 3 eq). The mixture was stirred at 25° C. for 15 mins. Then AcOH (287.71 mg, 4.79 mmol, 274.27 μL, 6 eq) and intermediate 1-2 (197.46 mg, 798.51 μmol, 1 eq) were added and the mixture was stirred at 25° C. for 15 mins. Then NaBH₃CN (200.72 mg, 3.19 mmol, 4 eq) was added and the mixture was stirred at 25° C. for 1 hr. The reaction mixture was concentrated under reduced pressure to give a residue. The crude product was purified by reversed-phase HPLC ( ) followed by lyophilization to give a residue. Intermediate 1-3 (210 mg, 212.30 μmol, 26.59% yield, 83% purity) was obtained as a light yellow oil. LCMS (Method H): Rt=0.554 min, (M+H)=821.2. Step 2: Synthesis of Intermediate 1-4

To a mixture of intermediate 1-3 (210 mg, 212.30 μmol, 1 eq) in DCM (2 mL) was added HCl/dioxane (2 M, 1 mL, 9.42 eq). The mixture was stirred at 25° C. for 30 mins. The reaction mixture was concentrated under reduced pressure to give a residue. Intermediate 1-4 (300 mg, crude, HCl salt) was obtained as a white solid. LCMS (Method D): Rt=0.241 min, (M+H)=721.5.

### Step 3: Synthesis of I-216

To a mixture of intermediate 1-4 (100 mg, 132.04 μmol, 1 eq, HCl salt) in DMF (1 mL) was added NMI (37.94 mg, 462.15 μmol, 36.84 μL, 3.5 eq). Then intermediate 1-5 (58.06 mg, 158.45 μmol, 1.2 eq) and TCFH (55.57 mg, 198.06 μmol, 1.5 eq) were added in and the mixture was stirred at 25° C. for 1 hour. The crude product was purified by reversed-phase HPLC (0.1% NH₃·H₂O condition) followed by lyophilization to give a residue. I-216 (22 mg, 20.50 μmol, 15.53% yield, 99.636% purity) was obtained as an off-white solid. LCMS (Method F): Rt=0.549 min, (M+H)=1070.0. SFC: Retention time: 0.876 min. ¹H NMR (400 MHz, METHANOL-d4) δ=8.97 (s, 1H), 8.63 (s, 1H), 8.36 (d, J=7.6 Hz, 1H), 7.99-7.91 (m, 1H), 7.90-7.80 (m, 2H), 7.79-7.66 (m, 2H), 7.51-7.34 (m, 4H), 7.16 (t, J=8.8 Hz, 1H), 4.59 (d, J=9.6 Hz, 2H), 4.38 (s, 2H), 4.23-4.11 (m, 4H), 4.05 (d, J=13.2 Hz, 1H), 3.91-3.82 (m, 1H), 3.81-3.70 (m, 4H), 3.68-3.60 (m, 9H), 3.57 (t, J=4.8 Hz, 3H), 3.51 (s, 1H), 3.24-3.12 (m, 3H), 3.07-2.96 (m, 1H), 2.95-2.64 (m, 5H), 2.59-2.35 (m, 9H), 2.19-2.11 (m, 2H), 2.09-1.99 (m, 3H), 1.91-1.80 (m, 2H), 1.76-1.59 (m, 3H), 1.55-1.45 (m, 1H), 1.30-1.12 (m, 2H).

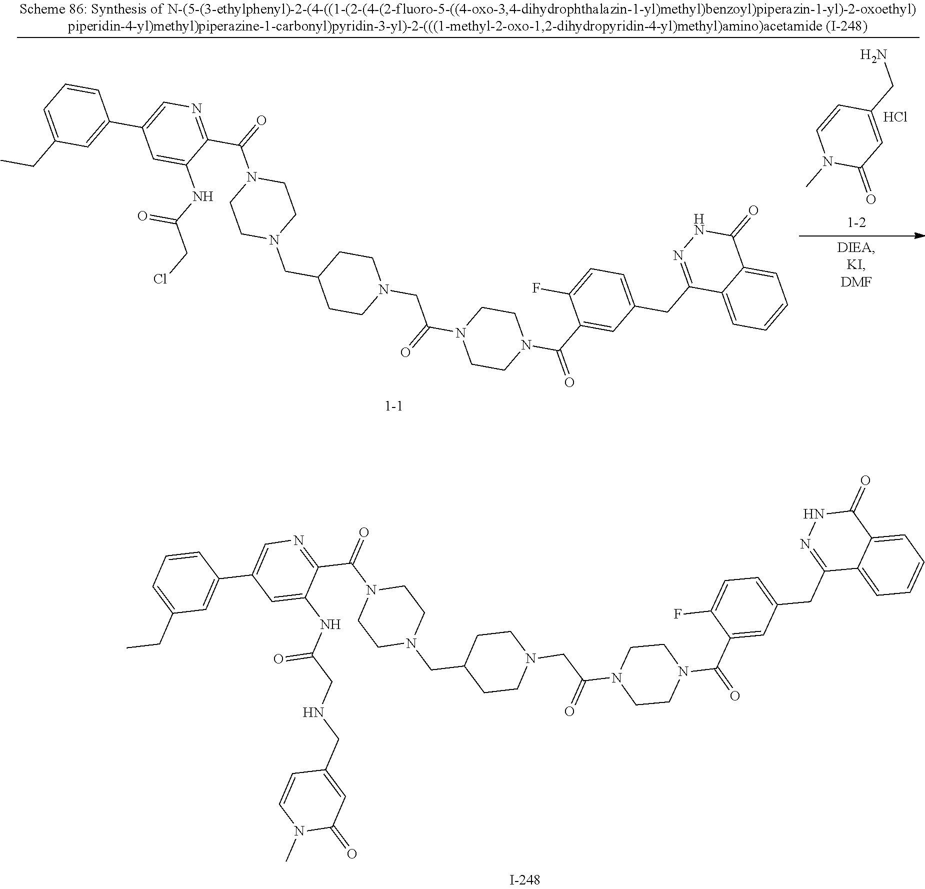

To a solution of Intermediate 1-1 (50 mg, 56.15 μmol, 1 eq) and Intermediate 1-2 (14.71 mg, 84.23 μmol, 1.5 eq, HCl salt) in DMF (1 mL) was added DIEA (36.29 mg, 280.76 μmol, 48.90 μL, 5 eq) and KI (37.29 mg, 224.61 μmol, 4 eq). The mixture was stirred at 30° C. for 12 hrs. The reaction mixture was concentrated under reduced pressure to give the crude product. The crude product was purified by prep-HPLC (column: Phenomenex luna C18 150*25 mm*10 um; mobile phase: [water (TFA)-ACN]; gradient: 10%-40% B over 15 min) and the eluent was concentrated to remove MeCN and then lyophilized to afford I-248 (32.05 mg, 28.97 μmol, 51.60% yield, 100% purity, TFA salt) as a white solid. LCMS (Method I): Rt=0.397 min, [M+H]⁺=992.7. ¹H NMR (400 MHz, METHANOL-d₄) δ=8.75-8.67 (m, 1H), 8.37 (d, J=7.6 Hz, 1H), 8.18 (d, J=1.6 Hz, 1H), 7.98-7.92 (m, 1H), 7.91-7.81 (m, 2H), 7.77 (d, J=7.2 Hz, 1H), 7.55-7.32 (m, 6H), 7.17-7.10 (m, 1H), 6.69 (s, 1H), 6.49-630 (m, 1H), 4.82-4.71 (m, 1H), 4.45-4.16 (m, 6H), 4.10 (s, 2H), 4.02-3.88 (m, 2H), 3.86-3.67 (m, 5H), 3.61-3.44 (m, 9H), 3.37 (s, 3H), 3.28-3.07 (m, 4H), 2.75-2.60 (m, 2H), 2.42-2.25 (m, 1H), 2.23-2.07 (m, 2H), 1.88-1.60 (m, 2H), 1.43-1.20 (m, 4H). ¹⁹F NMR (377 MHz, METHANOL-d₄), δ=−120.73.

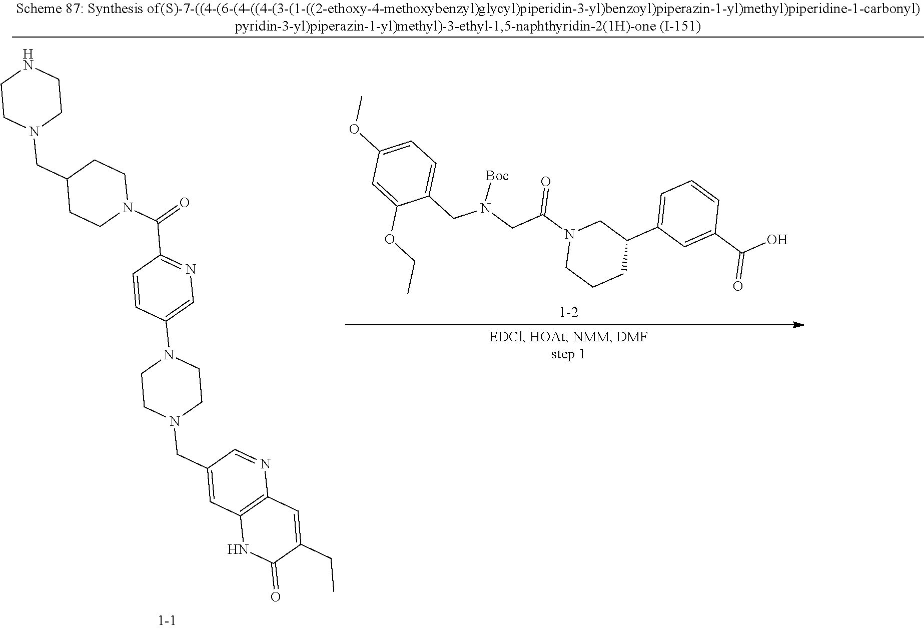 [SMILES: CCc1cc2ncc(CN3CCN(c4ccc(C(=O)N5CCC(CN6CCNCC6)CC5)nc4)CC3)cc2[nH]c1=O ; CCOc1cc(OC)ccc1CN(CC(=O)N1CCC[C@@H](c2cccc(C(=O)O)c2)C1)C(=O)OC(C)(C)C]

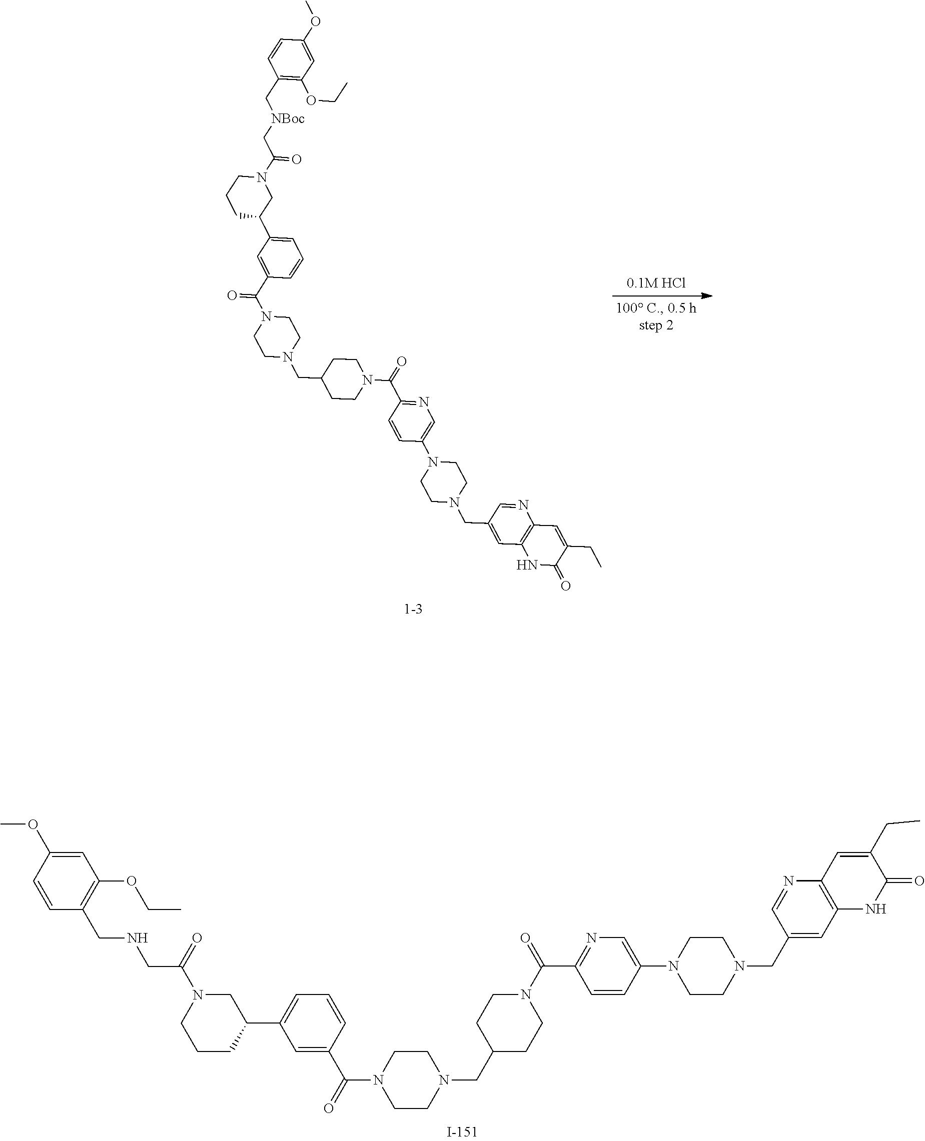 [SMILES: CCOc1cc(OC)ccc1CN(CC(=O)N1CCC[C@@H](c2cccc(C(=O)N3CCN(CC4CCN(C(=O)c5ccc(N6CCN(Cc7cnc8cc(CC)c(=O)[nH]c8c7)CC6)cn5)CC4)CC3)c2)C1)C(=O)OC(C)(C)C ; CCOc1cc(OC)ccc1CNCC(=O)N1CCC[C@@H](c2cccc(C(=O)N3CCN(CC4CCN(C(=O)c5ccc(N6CCN(Cc7cnc8cc(CC)c(=O)[nH]c8c7)CC6)cn5)CC4)CC3)c2)C1]

### Step 1: Synthesis of Intermediate 1-3

To a solution of Intermediate 1-1 (50 mg, 84.01 μmol, 1 eq, HCl salt) and Intermediate 1-2 (44.24 mg, 84.01 μmol, 1 eq) in DMF (0.5 mL) was added EDCI (48.31 mg, 252.03 μmol, 3 eq), HOAt (11.43 mg, 84.01 μmol, 1 eq) and NMM (84.97 mg, 840.09 μmol, 92.36 μL, 10 eq). The mixture was stirred at 25° C. for 1 hr. The mixture was poured into water (3 mL) and extracted with EA (3 mL*3). The combined organic layers were concentrated to give a residue. The residue was purified by flash silica gel chromatography (ISCO; 12 g SepaFlash Silica Flash Column, Eluent of 0-40% MeOH/Ethyl acetate gradient @20 mL/min) and the eluent was concentrated to give Intermediate 1-3 (85 mg, 77.03 μmol, 91.69% yield, 96.72% purity) as a white solid. LCMS (Method E): Rt=0.472 min, [M+H]⁺=1067.7.

### Step 2: Synthesis of I-151

To a solution of Intermediate 1-3 (85 mg, 79.64 μmol, 1 eq) was added HCl (0.1 M, 3.19 mL, 4 eq). The mixture was stirred at 100° C. for 0.5 hr. To the mixture was added NaHCO₃ to adjusted the pH to 9˜10, and then lyophilized to give a residue. The product was purified by prep-HPLC (column: Waters Xbridge 150*25 mm*5 um; mobile phase: [water (ammonia hydroxide v/v)-ACN]; gradient: 25%-55% B over 10 min) and the eluent was concentrated to remove MeCN and then lyophilization. I-151 (33.83 mg, 32.02 μmol, 40.21% yield, 91.55% purity) was obtained as an off-white solid. LCMS (Method G): Rt=0.658 min, [M+H]⁺=967.7. SFC: Rt=2.671 min, ee value=97.60%. ¹H NMR (400 MHz, METHANOL-d₄) δ =8.51-8.46 (m, 1H), 8.23 (d, J=2.4 Hz, 1H), 7.84-7.75 (m, 2H), 7.47-7.36 (m, 4H), 7.33-7.24 (m, 2H), 7.12-7.00 (m, 1H), 6.53-6.43 (m, 2H), 4.63-4.47 (m, 2H), 4.05-4.00 (m, 2H), 3.96-3.87 (m, 1H), 3.84-3.68 (m, 10H), 3.49-3.35 (m, 8H), 3.16-3.02 (m, 2H), 2.93-2.80 (m, 1H), 2.76-2.62 (m, 8H), 2.57-2.48 (m, 2H), 2.45-2.36 (m, 2H), 2.31-2.21 (m, 2H), 2.05-1.97 (m, 1H), 1.94-1.73 (m, 5H), 1.64-1.51 (m, 1H), 1.42-1.40 (m, 3H), 1.29-1.20 (m, 3H), 1.21-1.00 (m, 2H).

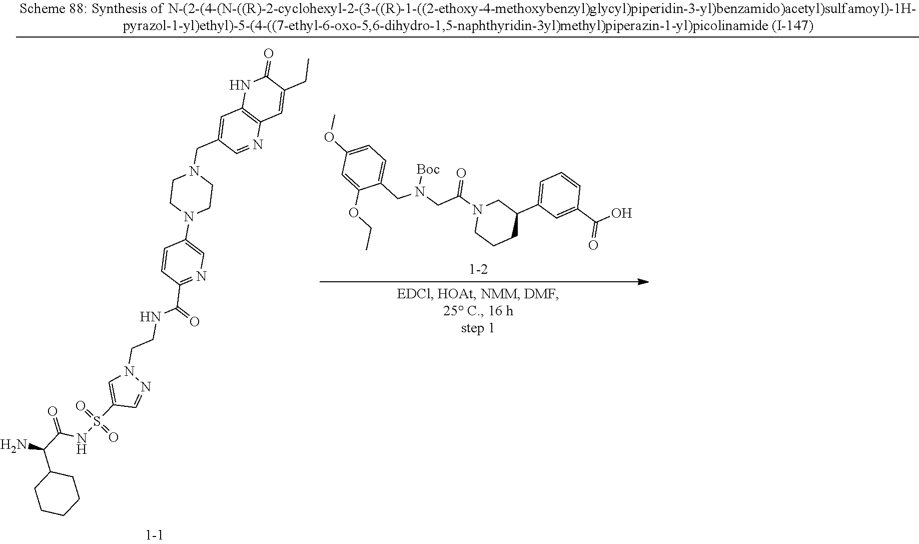 [SMILES: CCc1cc2ncc(CN3CCN(c4ccc(C(=O)NCCn5cc(S(=O)(=O)NC(=O)[C@H](N)C6CCCCC6)cn5)nc4)CC3)cc2[nH]c1=O ; CCOc1cc(OC)ccc1CN(CC(=O)N1CCC[C@H](c2cccc(C(=O)O)c2)C1)C(=O)OC(C)(C)C]

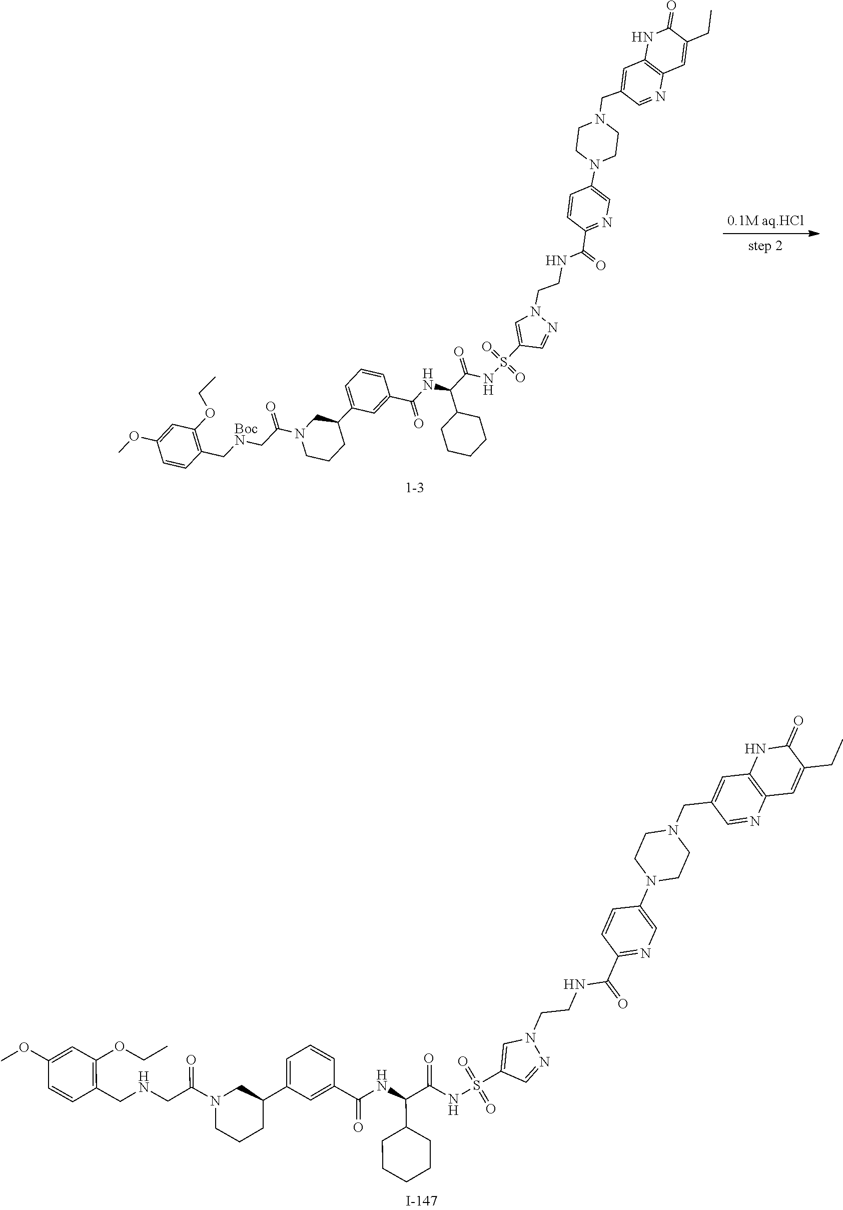

### Step 1: Synthesis of Intermediate 1-3

A mixture of Intermediate 1-1 (50 mg, 70.94 μmol, 1 eq), Intermediate 1-2 (29.89 mg, 56.75 mol, 0.8 eq) and HOAt (9.66 mg, 70.94 μmol, 1 eq) in DMF (1 mL) was added EDCI (27.20 mg, 141.88 mol, 2 eq) and NMM (35.88 mg, 354.69 μmol, 39.00 μL, 5 eq). The mixture was stirred at 25° C. for 1 hrs. The mixture was diluted with water (2 mL), and extracted with EtOAc (5 mL*3). The organic layer was concentrated under vacuum. The residue was purified by Prep-HPLC (0.1% FA condition) and dried by lyophilization to give Intermediate 1-3 (40 mg, 32.96 μmol, 46.47% yield) as white solid. LCMS (Method G): Rt=0.481 min, (M+H)+=1213.8.

### Step 2: Synthesis of I-147

A mixture of Intermediate 1-3 (40 mg, 32.96 μmol, 1 eq) in HCl (0.1 M, 0.5 mL, 1.52 eq). was stirred at 100° C. for 0.5 hr. The mixture was dried by lyophilization. The residue was purified by Prep-HPLC (column: Phenomenex luna C18 150*25 mm*10 um; mobile phase: [water(FA)-ACN]; gradient: 15%-45% B over 9 min) and dried by lyophilization to give I-147 (10.78 mg, 9.68 μmol, 29.37% yield, 100% purity) as white solid. LCMS (Method E): Rt=0.462 min, (M+H)+=1113.6. LCMS (Method): Rt=11.932 min, (M+H)+=1114.6. SFC: Rt=1.625 min, 1.847 min. ¹H NMR R (400 MHz, DMSO-d6) δ=11.96-11.74 (m, 1H), 8.54-8.53 (m, 1H), 8.40 (d, J=1.6 Hz, 1H), 8.26 (d, J=2.4 Hz, 1H), 8.06-7.95 (m, 1H), 7.82 (d, J=8.8 Hz, 1H), 7.77-7.62 (m, 5H), 7.48-7.24 (m, 4H), 6.60-6.50 (m, 2H), 4.48-4.30 (m, 1H), 4.26-4.25 (m, 2H), 4.14-3.85 (m, 7H), 3.75 (d, J=6.8 Hz, 3H), 3.70-3.60 (m, 5H), 3.28-3.05 (m, 5H), 2.92-2.70 (m, 2H), 2.56-2.52 (m, 7H), 2.00-1.88 (m, 1H), 1.84-1.70 (m, 3H), 1.65-1.43 (m, 6H), 1.42-1.26 (m, 4H), 1.19-1.17 (m, 3H), 1.14-0.84 (m, 6H).

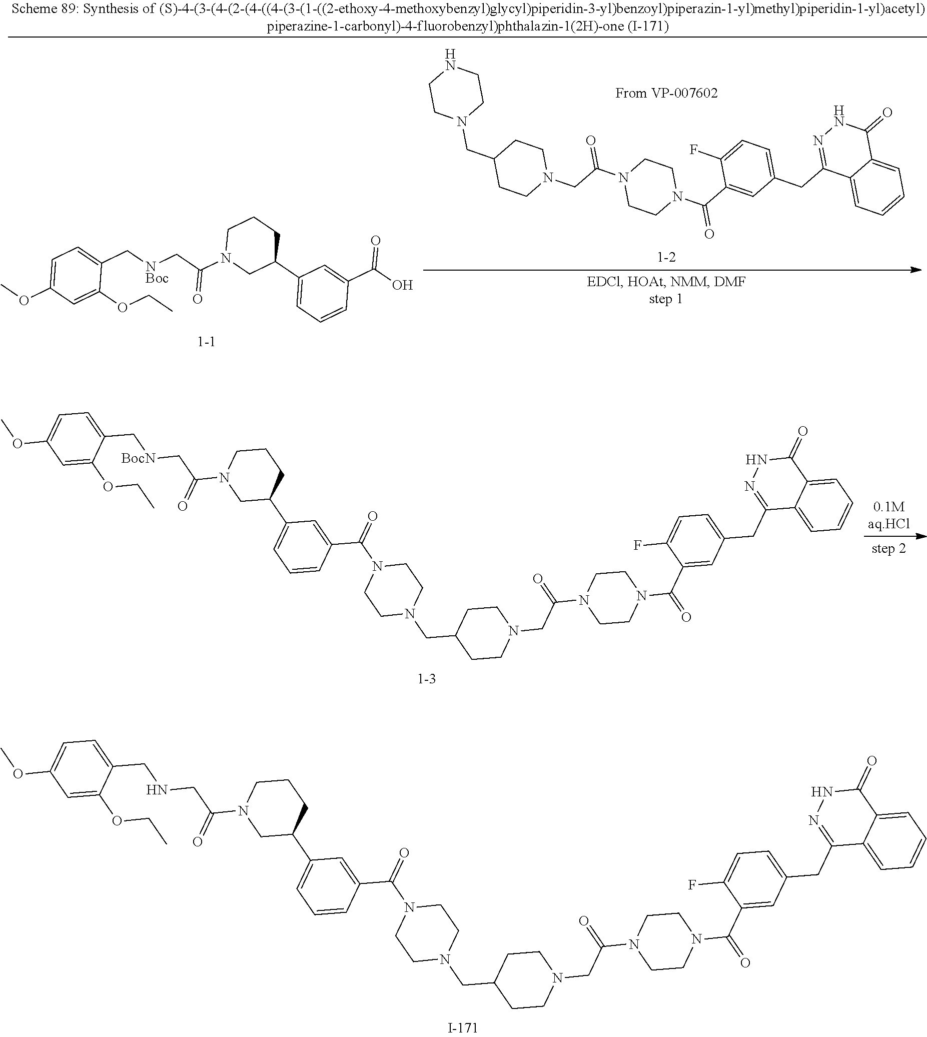 [SMILES: CCOc1cc(OC)ccc1CN(CC(=O)N1CCC[C@@H](c2cccc(C(=O)O)c2)C1)C(=O)OC(C)(C)C ; O=C(CN1CCC(CN2CCNCC2)CC1)N1CCN(C(=O)c2cc(Cc3n[nH]c(=O)c4ccccc34)ccc2F)CC1 ; CCOc1cc(OC)ccc1CN(CC(=O)N1CCC[C@@H](c2cccc(C(=O)N3CCN(CC4CCN(CC(=O)N5CCN(C(=O)c6cc(Cc7n[nH]c(=O)c8ccccc78)ccc6F)CC5)CC4)CC3)c2)C1)C(=O)OC(C)(C)C ; CCOc1cc(OC)ccc1CNCC(=O)N1CCC[C@@H](c2cccc(C(=O)N3CCN(CC4CCN(CC(=O)N5CCN(C(=O)c6cc(Cc7n[nH]c(=O)c8ccccc78)ccc6F)CC5)CC4)CC3)c2)C1]

### Step 1: Synthesis of Intermediate 1-3

To a solution of Intermediate 1-2 (71.34 mg, 113.93 μmol, 1.2 eq, HCl) and Intermediate 1-1 (50 mg, 94.95 μmol, 1 eq) in DMF (1 mL) was added EDCI (54.60 mg, 284.84 μmol, 3 eq), HOAt (12.92 mg, 94.95 μmol, 1 eq) and NMM (96.03 mg, 949.45 μmol, 104.38 μL, 10 eq). The mixture was stirred at 25° C. for 2 hrs. The reaction mixture was quenched by addition H₂O (1 mL), and then diluted with EA (1 mL) and extracted with EA (2 mL*3). The combined organic layers were washed with brine (2 mL*3), dried over anhydrous Na₂SO₄, filtered and concentrated under reduced pressure to give a residue. The residue was purified by reversed phase chromatography (0.10% FA condition), then concentrated to remove organic solvents and lyophilized to give Intermediate 1-3 (60 mg, 54.63 μmol, 57.54% yield, 100% purity) as a white solid. LCMS (Method E): Rt=0.476 min, [M+H]⁺=1098.6. SFC: Rt=3.228 min, ee value=97.14%.

### Step 2: Synthesis of I-171

To a solution of Intermediate 1-3 (60 mg, 54.63 μmol, 1 eq) in HCl (0.1 M, 1.64 mL, 3 eq) was stirred at 100° C. for 0.5 hr. The reaction mixture was adjusted to pH 8 with NH₃·H₂O, and then concentrated under reduced pressure to give a residue. The residue was purified by prep-HPLC (column: Waters Xbridge 150*25 mm*5 um; mobile phase: [water (ammonia hydroxide v/v)-ACN]; gradient: 33%-63% B over 10 min), then concentrated to remove organic solvents and lyophilized to give I-171 (25.05 mg, 24.84 μmol, 45.48% yield, 99% purity) as a white solid. LCMS (Method E): Rt=0.401 min, [M+H]⁺=998.8. SFC: Rt=4.808 min, ee value=94.71%. ¹H NMR (400 MHz, METHANOL-d₄) δ=8.39-8.34 (m, 1H), 7.97-7.92 (m, 1H), 7.90-7.80 (m, 2H), 7.48 (d, J=3.2 Hz, 1H), 7.45-7.35 (m, 3H), 7.32-7.25 (m, 2H), 7.18-7.10 (m, 2H), 6.53-6.41 (m, 2H), 4.56-4.47 (m, 1H), 4.38 (s, 2H), 4.11-4.00 (m, 2H), 3.87-3.63 (m, 13H), 3.59-3.34 (m, 7H), 3.25-3.16 (m, 2H), 3.14-3.03 (m, 1H), 2.94-2.84 (m, 2H), 2.77-2.62 (m, 2H), 2.54-2.35 (m, 4H), 2.25-2.21 (m, 2H), 2.10-1.98 (m, 3H), 1.87-1.72 (m, 4H), 1.62-1.50 (m, 2H), 1.45-1.39 (m, 3H), 1.31-1.15 (m, 2H).

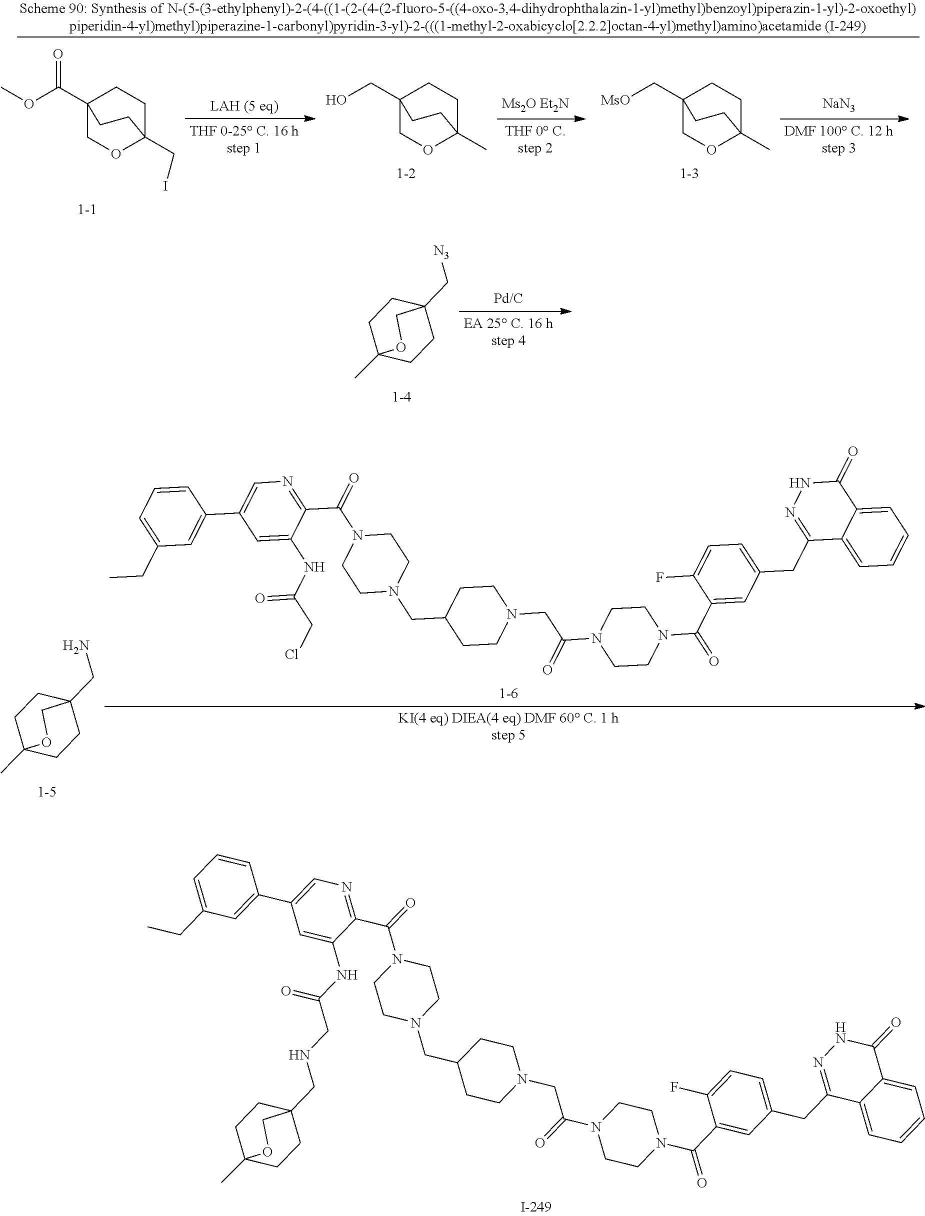

### Step 1: Synthesis of Intermediate 1-2

To a solution of Intermediate 1-1 (1 g, 3.22 mmol, 1 eq) in THF (10 mL) was added LAH (2.5 M, 6.45 mL, 5 eq) at 0° C. under N₂. The reaction mixture was stirred at 25° C. for 16 hrs under N₂. The reaction mixture was slowly added Na₂SO₄·10H₂O (5 g) at 0° C. The mixture was filtered and concentrated to afford crude product. The crude product was purified by flash silica gel chromatography (ISCO; 40 g SepaFlash Silica Flash Column, Eluent of 0˜100% Ethyl acetate/Petroleum ether gradient @40 mL/min) and concentrated under reduced pressure to give Intermediate 1-2 (290 mg, 1.86 mmol) as colorless oil. LCMS (Method I): Rt=0.387 min, [M+H]⁺=157.2. ¹H NMR (400 MHz, CHLOROFORM-d) δ=4.63 (s, 1H), 3.75 (s, 2H), 3.33-3.27 (m, 2H), 1.83-1.76 (m, 2H), 1.68-1.57 (m, 4H), 1.48 (d, J=6.4 Hz, 2H), 1.09 (s, 3H).

### Step 2: Synthesis of Intermediate 1-3

To a solution of Intermediate 1-2 (290 mg, 1.86 mmol, 1 eq) in THF (5 mL) was added TEA (563.52 mg, 5.57 mmol, 775.13 μL, 3 eq) and methylsulfonyl methanesulfonate (485.05 mg, 2.78 mmol, 1.5 eq). The mixture was stirred at 0° C. for 1 hr. The reaction was diluted with H₂O (5 mL), extracted with ethyl acetate 30 mL (10 mL*3). The organic phase was washed with saturated aqueous NaCl (5 mL). Then dried over Na₂SO₄, filtered and concentrated under reduced pressure to give a residue. The residue was purified by flash silica gel chromatography (ISCO; 20 g SepaFlash Silica Flash Column, Eluent of 0˜100% Ethyl acetate/Petroleum ether gradient @40 mL/min). The eluent was concentrated to afford Intermediate 1-3 (210 mg, 896.24 μmol, 48.28% yield) as a white solid. ¹H NMR (400 MHz, CHLOROFORM-d) δ=3.90 (s, 2H), 3.77 (s, 2H), 3.00 (d, J=0.8 Hz, 3H), 1.92-1.79 (m, 2H), 1.72 (br t, J=11.6 Hz, 2H), 1.68-1.59 (m, 3H), 1.58-1.50 (m, 1H), 1.12 (s, 3H).

### Step 3: Synthesis of Intermediate 1-4

To a solution of Intermediate 1-3 (150 mg, 640.17 μmol, 1 eq) in DMF (2 mL) was added NaN₃ (200 mg, 3.08 mmol, 4.81 eq). The mixture was stirred at 100° C. for 12 hrs. The reaction was diluted with sat. Na₂CO₃ (5 mL), extracted with ethyl acetate (10 mL*3). The organic phase was washed with saturated aqueous NaCl (5 mL), dried over Na₂SO₄, filtered and concentrated under reduced pressure to give a residue. The crude product Intermediate 1-4 (200 mg) was obtained as colorless oil and it was used in the next step directly without further purification. ¹H NMR (400 MHz, CHLOROFORM-d) δ=3.68 (t, J=1.2 Hz, 2H), 3.09-3.03 (m, 2H), 1.87-1.72 (m, 2H), 1.68-1.46 (m, 6H), 1.07 (s, 3H).

### Step 4: Synthesis of Intermediate 1-5

To a solution of Pd/C (23.49 mg, 22.07 μmol, 10% purity, 0.2 eq) in EA (2 mL) was added Intermediate 1-4 (20 mg, 110.35 μmol, 1 eq) under N₂. The suspension was degassed under vacuum and purged with H₂ several times. The mixture was stirred under H₂ (15 psi) at 25° C. for 16 hours. The reaction was diluted with MeOH (3 mL*5) filtered and concentrated under reduced pressure to give Intermediate 1-5 (10 mg, 64.42 μmol, 58.37% yield) as a white solid and it was used in the next step without further purification. MS: [M+H]⁺=156.2.

### Step 5: Synthesis of I-249

To a solution of Intermediate 1-5 (5.23 mg, 33.69 μmol, 1.2 eq) and Intermediate 1-6 (25 mg, 28.08 μmol, 1 eq) in DMF (1 mL) was added KI (18.64 mg, 112.30 μmol, 4 eq) and DIEA (14.51 mg, 112.30 μmol, 19.56 μL, 4 eq). The mixture was stirred at 60° C. for 1 hr. The reaction was filtered and the filtrate was purified by prep-HPLC (column: YMC-Actus Triart C18 150*30 mm*7 um; mobile phase: [water (FA)-ACN]; gradient: 13%-43% B over 10 min). The eluent was concentrated under reduced pressure to remove ACN, then lyophilized to afford I-249 (12 mg, 11.22 μmol, 39.97% yield, 98.69% purity, FA salt) as a white solid. LCMS (Method I): Rt=0.422 min, [M+H]⁺=1009.5. ¹H NMR (400 MHz, METHANOL-d₄) δ=8.79 (s, 1H), 8.63 (d, J=2.0 Hz, 1H), 8.43-8.35 (m, 1H), 8.32-8.27 (m, 1H), 8.01-7.95 (m, 1H), 7.93-7.77 (m, 2H), 7.66-7.31 (m, 7H), 7.21-7.17 (m, 1H), 4.60 (br s, 2H), 4.41 (s, 2H), 4.25-4.12 (m, 2H), 3.92-3.83 (m, 5H), 3.81-3.72 (m, 2H), 3.58 (br s, 5H), 3.50 (s, 2H), 3.41 (br s, 1H), 3.11-2.92 (m, 2H), 2.79-2.73 (m, 2H), 2.70-2.64 (m, 2H), 2.61-2.54 (m, 2H), 2.51 (s, 2H), 2.39 (d, J=6.5 Hz, 2H), 2.07 (d, J=13.2 Hz, 2H), 2.01-1.91 (m, 1H), 1.88-1.80 (m, 2H), 1.78-1.67 (m, 6H), 1.62-1.50 (m, 2H), 1.31-1.29 (m, 3H), 1.11 (s, 3H). ¹⁹F NMR (377 Hz, METHANOL-d₄), δ=−120.69.

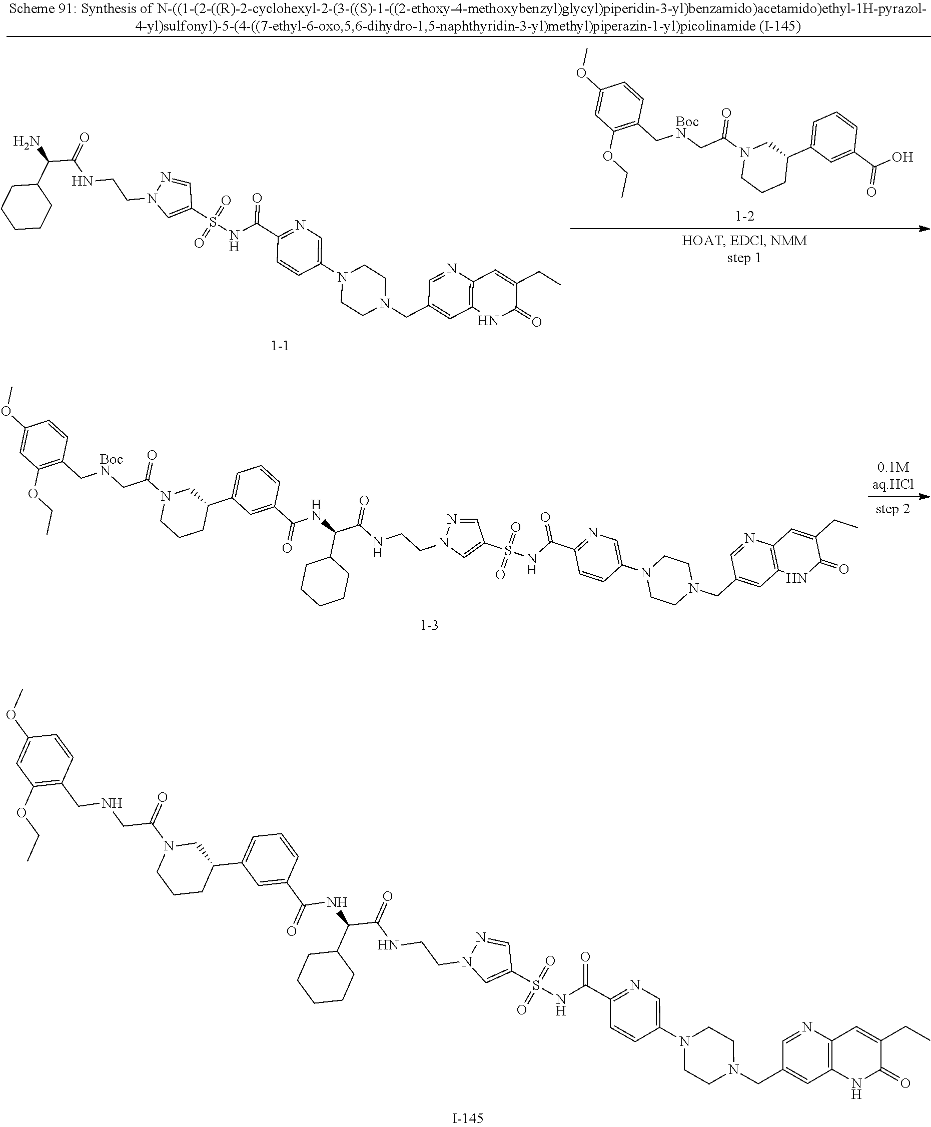 [SMILES: CCc1cc2ncc(CN3CCN(c4ccc(C(=O)NS(=O)(=O)c5cnn(CCNC(=O)[C@H](N)C6CCCCC6)c5)nc4)CC3)cc2[nH]c1=O ; CCOc1cc(OC)ccc1CN(CC(=O)N1CCC[C@@H](c2cccc(C(=O)O)c2)C1)C(=O)OC(C)(C)C ; CCOc1cc(OC)ccc1CN(CC(=O)N1CCC[C@@H](c2cccc(C(=O)N[C@@H](C(=O)NCCn3cc(S(=O)(=O)NC(=O)c4ccc(N5CCN(Cc6cnc7cc(CC)c(=O)[nH]c7c6)CC5)cn4)cn3)C3CCCCC3)c2)C1)C(=O)OC(C)(C)C ; CCOc1cc(OC)ccc1CNCC(=O)N1CCC[C@@H](c2cccc(C(=O)N[C@@H](C(=O)NCCn3cc(S(=O)(=O)NC(=O)c4ccc(N5CCN(Cc6cnc7cc(CC)c(=O)[nH]c7c6)CC5)cn4)cn3)C3CCCCC3)c2)C1]

### Step 1: Synthesis of Intermediate 1-2

A mixture of Intermediate 1-1 (65 mg, 87.68 μmol, 1 eq, HCl), Intermediate 1-2 (46.18 mg, 87.68 μmol, 1 eq) and HOAt (11.93 mg, 87.68 μmol, 1 eq) in DMF (1 mL) was added EDCI (33.62 mg, 175.37 μmol, 2 eq) and NMM (44.35 mg, 438.42 μmol, 48.20 μL, 5 eq). The mixture was stirred at 25° C. for 1 hrs. The mixture was diluted with water (2 mL), and extracted with EtOAc (5 mL*3). The organic layer was concentrated under vacuum. The residue was purified by Pre-HPLC (column: Phenomenex luna C18 150*25 mm*10 um; mobile phase: [water(FA)-ACN]; gradient: 35%-65% B over 9 min) and dried by lyophilization to give Intermediate 1-3 (40 mg, 32.96 μmol, 37.59% yield) as white solid. LCMS (Method E): Rt=0.544 min, (M+H)+=1213.6. SFC: Rt=1.669 min, 1.753 min.

### Step 2: Synthesis of I-145

A mixture of Intermediate 1-3 (40.00 mg, 32.96 μmol, 1 eq) in HCl (0.1 M, 1 mL, 3.03 eq) was stirred at 100° C. for 2 hr. The mixture was dried by lyophilization. The residue was purified by Pre-HPLC (column: Phenomenex luna C18 150*25 mm*10 um; mobile phase: [water(FA)-ACN]; gradient: 15%-45% B over 9 min) and dried by lyophilization to give I-145 (12.58 mg, 11.30 μmol, 34.28% yield, 100% purity) as white solid. LCMS (Method E): Rt=0.435 min, (M+H)+=1113.6. SFC: Rt=2.597 min, 3.212 min. ¹H NMR (400 MHz, DMSO-d6) δ=12.00-11.70 (m, 1H), 8.42-8.28 (m, 2H), 8.21-8.12 (m, 1H), 8.11-7.97 (m, 2H), 7.91-7.80 (m, 1H), 7.80-7.39 (m, 7H), 7.33-7.17 (m, 2H), 6.57-6.37 (m, 2H), 4.49-4.35 (m, 1H), 4.29-4.11 (m, 4H), 4.09-3.86 (m, 6H), 3.78-3.62 (m, 8H), 3.23 (br d, J=2.4 Hz, 4H), 2.90-2.76 (m, 2H), 2.65-2.59 (m, 1H), 2.53 (br d, J=2.0 Hz, 4H), 1.94-1.86 (m, 1H), 1.84-1.58 (m, 7H), 1.56-1.41 (m, 3H), 1.37-1.22 (m, 4H), 1.21-0.86 (m, 9H).

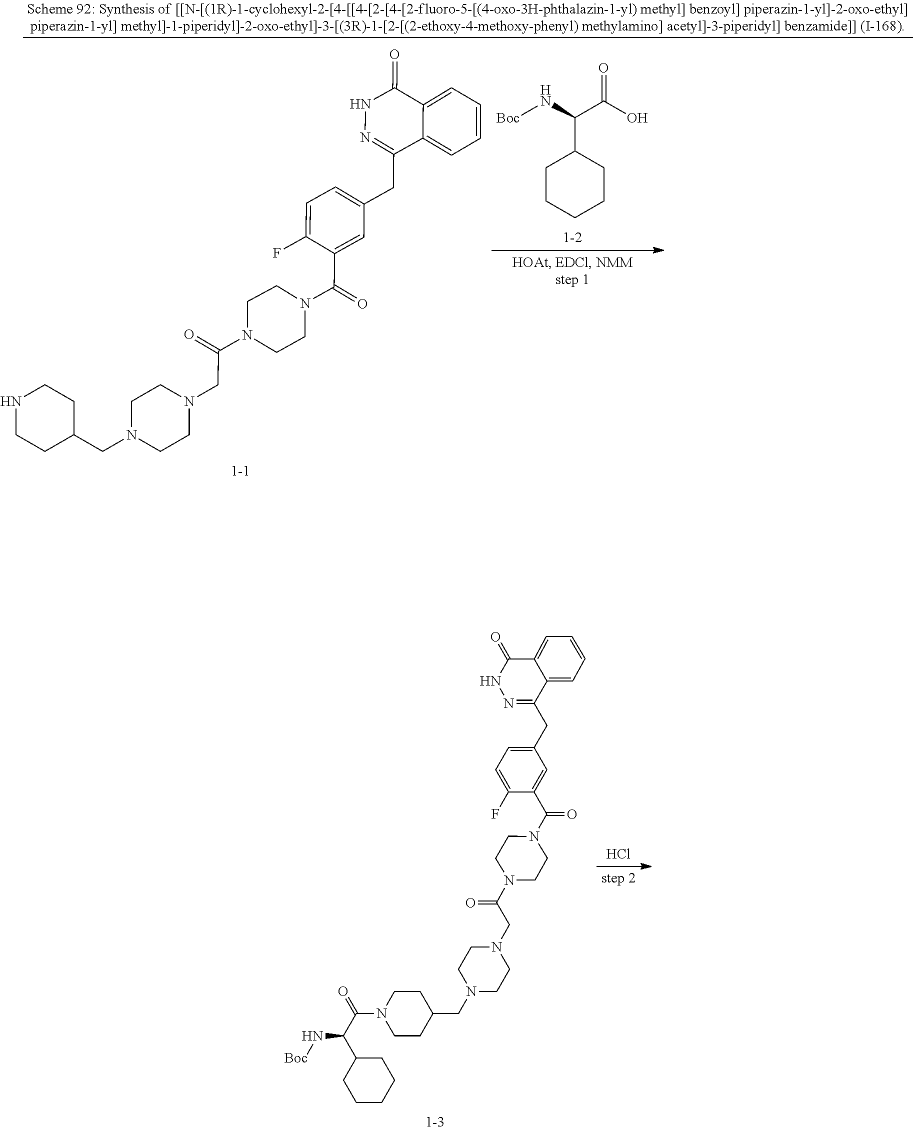

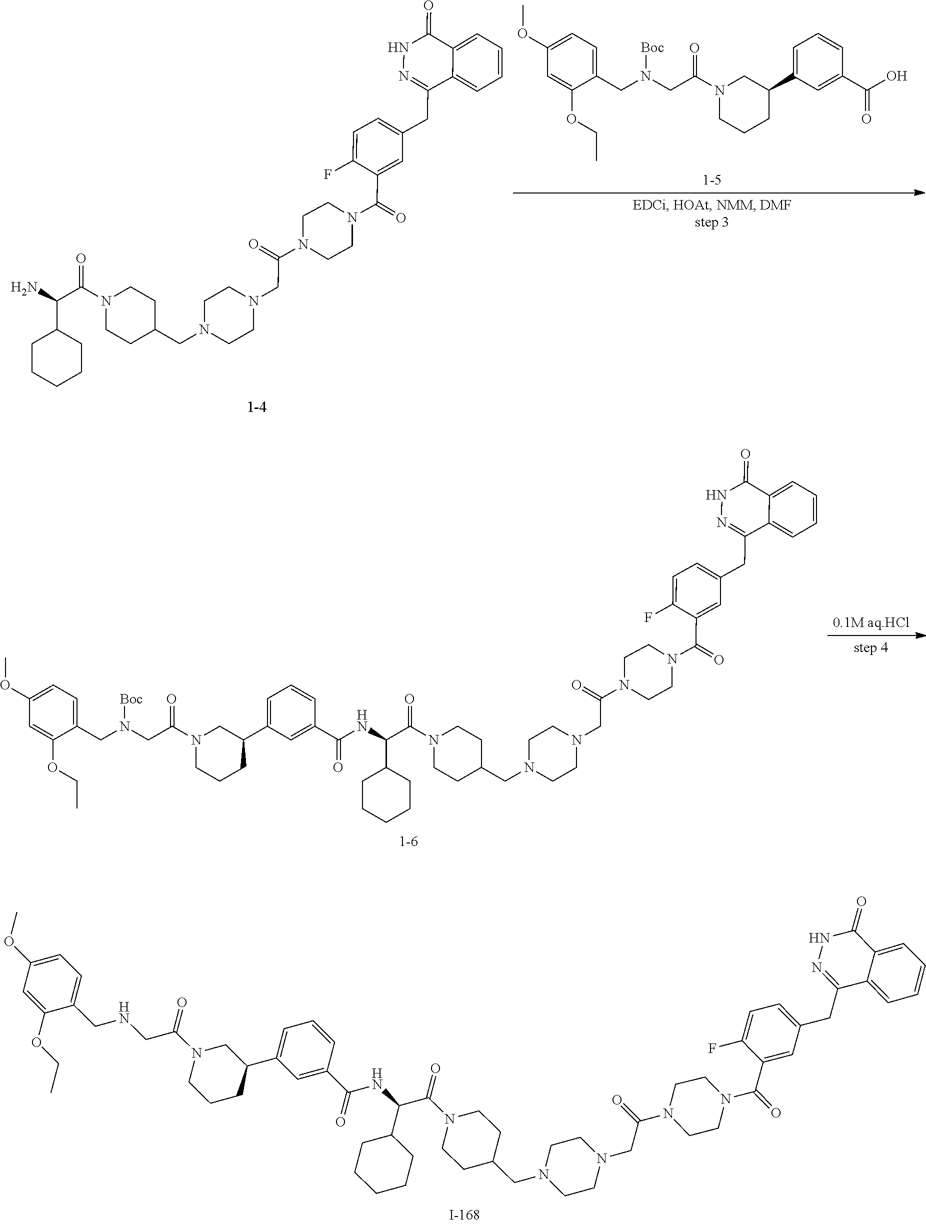

### Step 1: Synthesis of Intermediate 1-3

To a solution of intermediate 1-1 (1.8 g, 2.87 mmol, 1 eq, HCl) and intermediate 1-2 (887.66 mg, 3.45 mmol, 1.2 eq) in DMF (18 mL) was added EDCI (1.65 g, 8.62 mmol, 3 eq), NMM (1.45 g, 14.37 mmol, 1.58 mL, 5 eq) and HOAt (391.27 mg, 2.87 mmol, 402.13 μL, 1 eq). The mixture was stirred at 25° C. for 1 hr. The reaction mixture was poured into H₂O (50 mL), and extracted with EA (20 mL*3). The combined organic layers were dried over Na₂SO4, filtered and concentrated under reduced pressure to give a residue. The residue was purified by flash silica gel chromatography (ISCO; 40 g SepaFlash Silica Flash Column, Eluent of 0˜100% PE/EA@36 mL/min, PE/EA=0:1, Rf=0.2) and the eluent was concentrated under reduced pressure to give a product. Intermediate 1-3 (2.5 g, crude) was obtained as a yellow solid. LCMS (Method D): Rt=0.370 min, (M+H)=829.4.

### Step 2: Synthesis of Intermediate 1-4

To a solution of intermediate 1-3 (2.5 g, 3.02 mmol, 1 eq) in DCM (5 mL) was added HCl/dioxane (2 M, 20 mL, 13.26 eq) and it was stirred at 25° C. for 0.5 hr. The reaction mixture was concentrated under reduced pressure to give a residue. Intermediate 1-4 (2.6 g, crude, HCl salt) was obtained as a yellow solid. LCMS (Method D): Rt=0.268 min, (M+H)=729.3.

### Step 3: Synthesis of Intermediate 1-6

To a solution of intermediate 1-4 (500 mg, 653.29 μmol, 1 eq, HCl salt) and intermediate 1-5 (412.84 mg, 783.95 μmol, 1.2 eq) in DMF (5 mL) was added EDCI (375.71 mg, 1.96 mmol, 3 eq), NMM (330.39 mg, 3.27 mmol, 359.12 μL, 5 eq) and HOAt (88.92 mg, 653.29 μmol, 91.39 μL, 1 eq). The mixture was stirred at 25° C. for 0.5 hr. The reaction mixture was poured into H₂O (20 mL), and extracted with EA (10 mL*3). The combined organic layers were dried over Na₂SO₄, filtered and concentrated under reduced pressure to give a residue. The residue was purified by flash silica gel chromatography (ISCO; 20 g SepaFlash Silica Flash Column, Eluent of 0˜100% DCM/MeOH@50 mL/min, DCM/MeOH=10:1, Rf=0.2) and the eluent was concentrated under reduced pressure to give a residue. Intermediate 1-6 (478 mg, crude) was obtained as a yellow gum. LCMS (Method D): Rt=0.460 min, (M+H)=1237.8. SFC: Retention time: 0.952 min.

### Step 4: Synthesis of I-168

To a solution of intermediate 1-6 (478 mg, 386.26 μmol, 1 eq) was added HCl (0.1 M, 15.45 mL, 4 eq). The mixture was stirred at 100° C. for 0.5 hr. To the mixture was added NaHCO₃ to adjust the pH to 9˜10, and then it was extracted with DCM (50 mL*3), the combined organic layers were dried over Na₂SO₄, filtered and concentrated under reduced pressure to give a residue. The residue was purified by prep-HPLC (column: Waters Xbridge 150*25 mm*5 um; mobile phase: [water (ammonia hydroxide v/v)-ACN]; gradient: 38%-68% B over 10 min) and the eluent was concentrated to remove MeCN and then lyophilized. I-168 (124.76 mg, 108.30 μmol, 28.04% yield, 98.730% purity) was obtained as a white solid. LCMS (Method D): Rt=0.368 min, (M+H)=1137.9. SFC: Retention time: 3.910 min. ¹H NMR (400 MHz, METHANOL-d4) 6=8.49-8.25 (m, 1H), 8.03-7.91 (m, 1H), 7.90-7.79 (m, 2H), 7.79-7.65 (m, 2H), 7.58-7.32 (m, 4H), 7.25-7.04 (m, 2H), 6.67-6.36 (m, 2H), 4.59-4.50 (m, 2H), 4.39 (s, 2H), 4.34-4.24 (m, 1H), 4.05 (dd, J=6.8, 13.4 Hz, 2H), 3.87-3.62 (m, 11H), 3.60-3.44 (m, 4H), 3.26-3.04 (m, 4H), 2.81-2.62 (m, 4H), 2.61-2.33 (m, 8H), 2.28-2.14 (m, 2H), 2.02 (dd, J=3.6, 9.8 Hz, 1H), 1.97-1.74 (m, 10H), 1.72-1.62 (m, 2H), 1.47-1.38 (m, 3H), 1.34-1.02 (m, 8H). ¹⁹F NMR (400 MHz, CHLOROFORM-d) δ=−120.808.

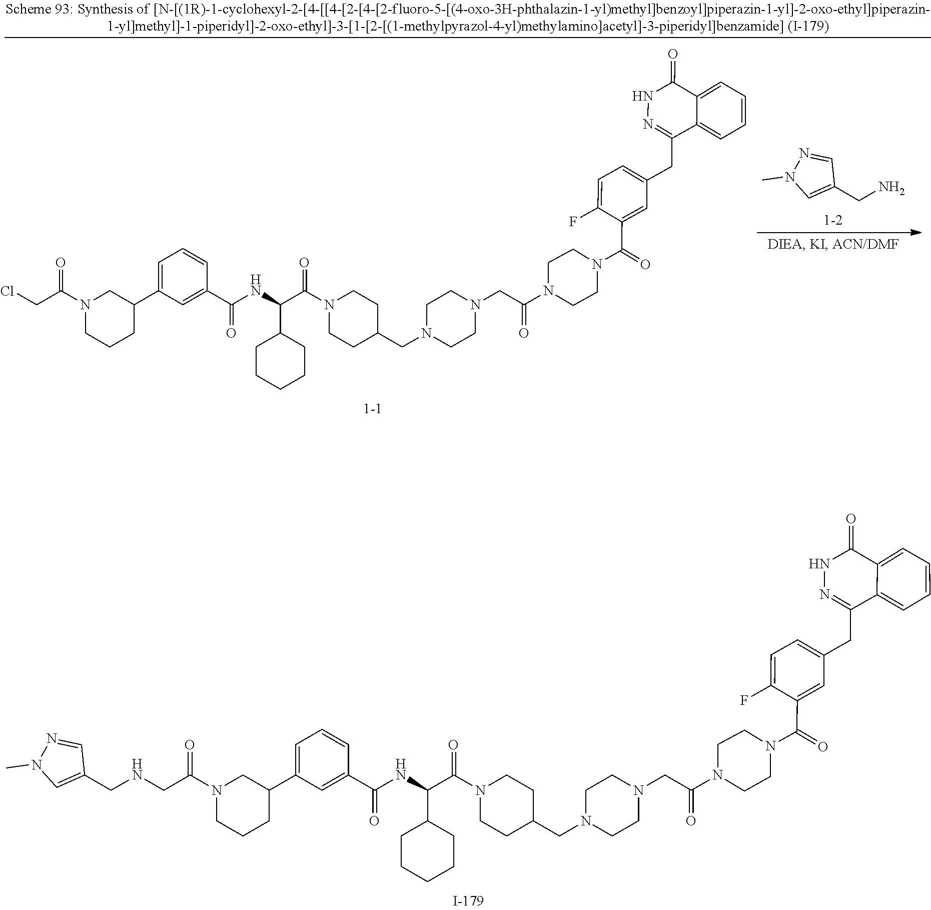

To a solution of intermediate 1-2 (5.04 mg, 45.33 μmol, 1.5 eq) in ACN (0.2 mL) and DMF (0.2 mL) was added DIEA (15.62 mg, 120.89 μmol, 21.06 μL, 4 eq) at 25° C. under N₂ and the mixture was stirred at 25° C. for 0.5 h, then intermediate 1-1 (30 mg, 30.22 μmol, 1 eq) and KI (2.51 mg, 15.11 μmol, 0.5 eq) were added to the mixture and it was stirred at 60° C. for 2 hr. The reaction mixture was concentrated to give a residue. The residue was purified by reversed-phase HPLC (column: Waters Xbridge 150*25 mm*5 um; mobile phase: [water (NH₃H2O)-ACN]; gradient: 28%-58% B over 10 min) and the eluent was lyophilized to give I-179 (8.09 mg, 7.46 μmol, 24.68% yield, 98.419% purity) as a white solid. LCMS (Method D): Retention time: 0.343 min, (M+1)=1067.6. SFC: Retention time: 6.015 min, 8.013 min. ¹H NMR (400 MHz, CHLOROFORM-d) δ=10.20-9.82 (m, 1H), 8.52-8.40 (m, 1H), 7.83-7.63 (m, 5H), 7.46-7.28 (m, 6H), 7.14-7.02 (m, 2H), 5.07 (d, J=1.6 Hz, 1H), 4.76-4.66 (m, 1H), 4.65-4.56 (m, 1H), 4.29 (s, 2H), 4.15-4.03 (m, 1H), 3.87 (d, J=11.6 Hz, 3H), 3.82-3.75 (m, 2H), 3.70 (d, J=8.4 Hz, 4H), 3.64-3.54 (m, 2H), 3.52-3.43 (m, 2H), 3.42-2.97 (m, 7H), 2.76-2.59 (m, 3H), 2.59-2.31 (m, 8H), 2.25-2.12 (m, 2H), 2.11-2.03 (m, 1H), 1.93-1.72 (m, 11H), 1.41-0.95 (m, 9H). ¹⁹F NMR (376 MHz, METHANOL-d4) δ=−117.618.

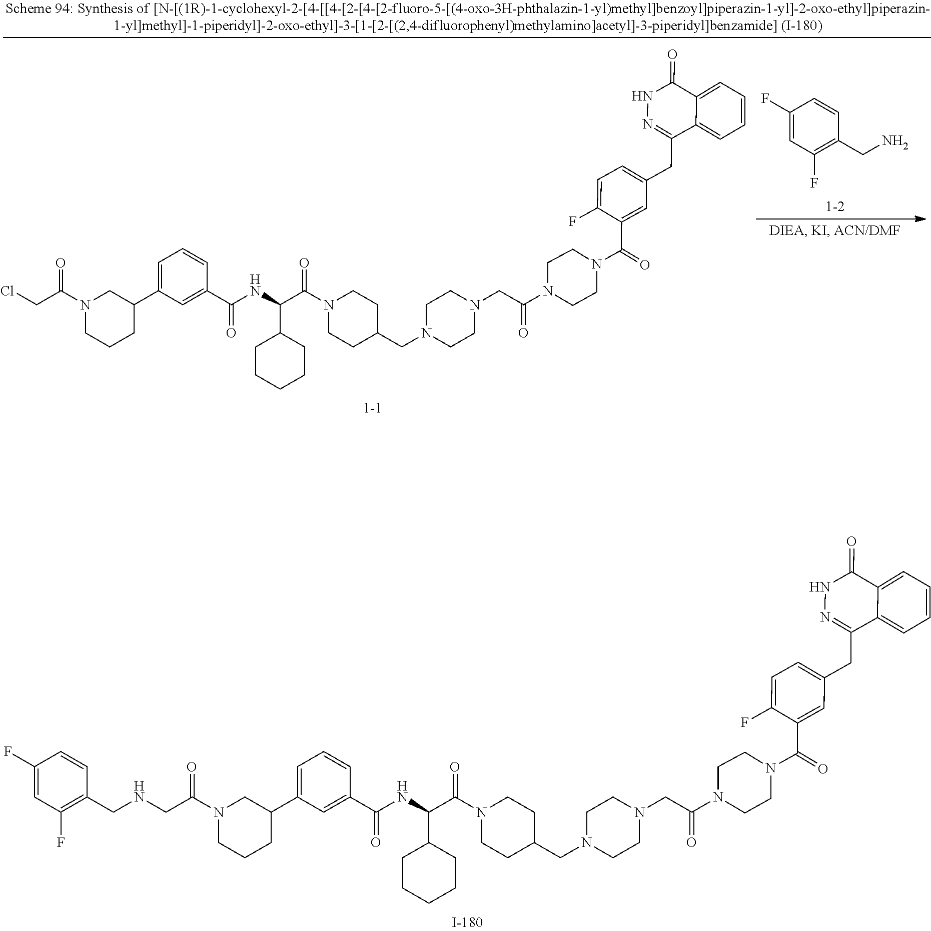

To a solution of intermediate 1-2 (6.49 mg, 45.33 μmol, 5.39 μL, 1.5 eq) in ACN (0.2 mL) and DMF (0.2 mL) was added DIEA (15.62 mg, 120.89 μmol, 21.06 μL, 4 eq) at 25° C. under N₂ and stirred at 25° C. for 0.5 h, then intermediate 1-1 (30 mg, 30.22 μmol, 1 eq) and KI (2.51 mg, 15.11 μmol, 0.5 eq) were added to the mixture and it was stirred at 60° C. for 2 h. The reaction mixture was concentrated to give a residue. The residue was purified by reversed-phase HPLC (column: Waters Xbridge 150*25 mm*5 um; mobile phase: [water (NH₃H2O)-ACN]; gradient: 38%-68% B over 10 min) and lyophilized to give I-180 (6.3 mg, 5.65 μmol, 18.68% yield, 98.536% purity) as a white solid. LCMS (Method D): Retention time: 0.364 min, (M+1)=1099.5. SFC: Retention time: 4.174 min, 5.637 min. ¹H NMR (400 MHz, CHLOROFORM-d) δ=10.11-9.79 (m, 1H), 8.51-8.42 (m, 1H), 7.84-7.62 (m, 5H), 7.44-7.30 (m, 5H), 7.14-7.02 (m, 2H), 6.91-6.74 (m, 2H), 5.14-5.02 (m, 1H), 4.78-4.66 (m, 1H), 4.65-4.54 (m, 1H), 4.29 (s, 2H), 4.15-4.03 (m, 1H), 3.84 (d, J=9.2 Hz, 2H), 3.81-3.68 (m, 4H), 3.65-3.53 (m, 2H), 3.53-3.44 (m, 2H), 3.42-2.96 (m, 7H), 2.77-2.60 (m, 3H), 2.58-2.32 (m, 8H), 2.25-2.12 (m, 2H), 2.11-2.03 (m, 1H), 1.98-1.69 (m, 12H), 1.33-1.00 (m, 8H)¹⁹FNMR: (376 MHz, METHANOL-d4) δ=−112.049, −114.871, −117.618.

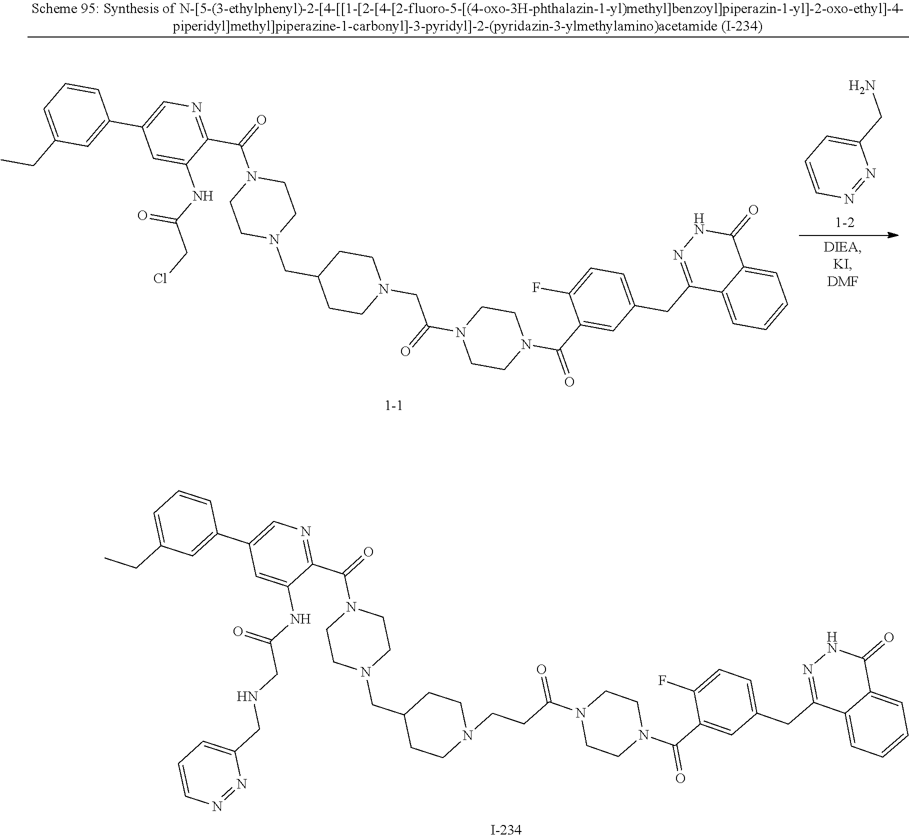 [SMILES: CCc1cccc(-c2cnc(C(=O)N3CCN(CC4CCN(CC(=O)N5CCN(C(=O)c6cc(Cc7n[nH]c(=O)c8ccccc78)ccc6F)CC5)CC4)CC3)c(NC(=O)CCl)c2)c1 ; NCc1cccnn1 ; CCc1cccc(-c2cnc(C(=O)N3CCN(CC4CCN(CCC(=O)N5CCN(C(=O)c6cc(Cc7n[nH]c(=O)c8ccccc78)ccc6F)CC5)CC4)CC3)c(NC(=O)CNCc3cccnn3)c2)c1]

To a solution of Intermediate 1-1 (50 mg, 56.15 μmol, 1 eq) and Intermediate 1-2 (9.19 mg, 84.23 μmol, 1.5 eq) in DMF (1 mL) was added DIEA (36.29 mg, 280.76 μmol, 48.90 μL, 5 eq) and KI (37.29 mg, 224.61 μmol, 4 eq). The mixture was stirred at 60° C. for 2 hrs. The reaction mixture was washed with water and extracted with EA 3 times, and the separated organic phase was concentrated to give the crude product. The crude product was purified by prep-HPLC (column: YMC-Actus Triart C18 150*30 mm*7 um; mobile phase: [water (FA)-ACN]; gradient: 8%-38% B over 10 min) and the eluent was concentrated to remove MeCN and then lyophilized to afford I-234 (10.19 mg, 9.90 μmol, 17.64% yield, 98.07% purity, FA salt) as an orange solid. LCMS: Rt=0.429 min, [M+H]⁺=963.6. ¹H NMR (400 MHz, METHANOL-d₄) δ=9.12-9.10 (m, 1H), 8.73 (d, J=2.0 Hz, 1H), 8.61 (d, J=2.0 Hz, 1H), 8.37-8.30 (m, 1H), 8.26-8.24 (m, 1H), 8.04-8.00 (m, 1H), 7.98-7.93 (m, 1H), 7.92-7.81 (m, 2H), 7.77-7.60 (m, 1H), 7.57-7.48 (m, 3H), 7.46-7.35 (m, 2H), 7.32 (d, J=7.6 Hz, 1H), 7.17-7.10 (m, 1H), 4.69-4.55 (m, 1H), 4.39 (s, 2H), 4.26-4.14 (m, 4H), 3.85-3.69 (m, 5H), 3.60-3.49 (m, 8H), 3.40 (s, 2H), 3.10-2.98 (m, 2H), 2.75-2.65 (m, 2H), 2.54 (d, J=4.4 Hz, 4H), 2.35 (d, J=6.8 Hz, 2H), 2.09-1.99 (m, 2H), 1.98-1.86 (m, 1H), 1.61-1.46 (m, 2H), 1.29-1.20 (m, 3H). ¹⁹F NMR (377 MHz, METHANOL-d₄), δ=−120.743.

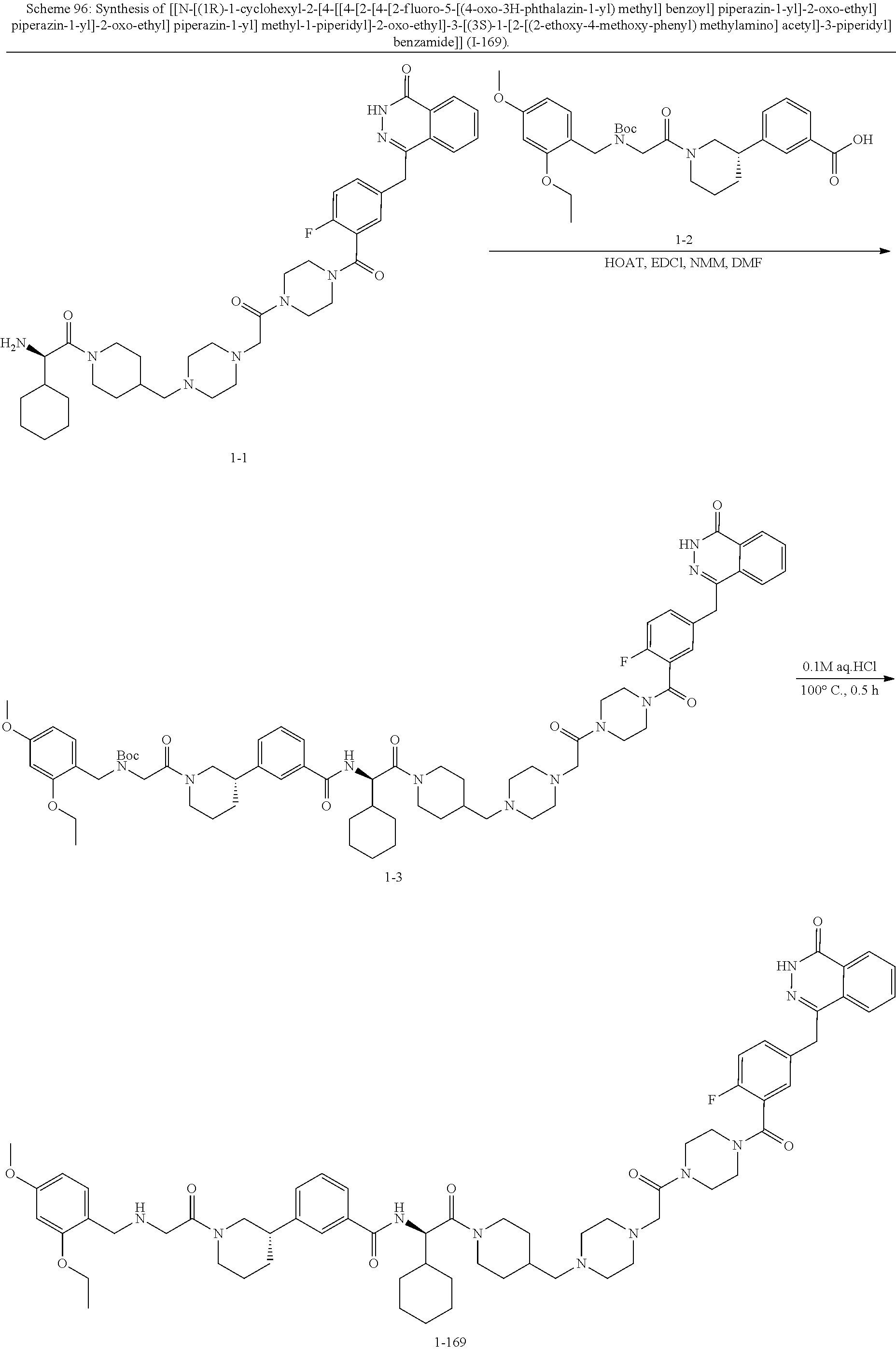

### Step 1: Synthesis of Intermediate 1-3

To a solution of intermediate 1-1 (500 mg, 653.29 μmol, 1 eq, HCl salt) and intermediate 1-2 (412.84 mg, 783.95 μmol, 1.2 eq) in DMF (5 mL) was added EDCI (375.71 mg, 1.96 mmol, 3 eq), NMM (330.39 mg, 3.27 mmol, 359.12 μL, 5 eq) and HOAt (88.92 mg, 653.29 μmol, 91.39 μL, 1 eq). The mixture was stirred at 25° C. for 0.5 hr. The reaction mixture was poured into H₂O (20 mL), and extracted with EA (10 mL*3). The combined organic layers were dried over Na₂SO₄, filtered and concentrated under reduced pressure to give a residue. The residue was purified by flash silica gel chromatography (ISCO; 20 g SepaFlash Silica Flash Column, Eluent of 0˜100% DCM/MeOH@50 mL/min, DCM/MeOH=10:1, Rf=0.2) and the eluent was concentrated under reduced pressure to give Intermediate 1-3 (565 mg, crude) as a yellow gum. LCMS (Method D): Rt=0.455 min, (M+H)=1237.5. SFC: Retention time: 0.991 min.

### Step 2: Synthesis of I-169

To a solution of intermediate 1-3 (565 mg, 456.57 μmol, 1 eq) was added HCl (0.1 M, 18.26 mL, 4 eq). The mixture was stirred at 100° C. for 0.5 hr. To the mixture was added NaHCO₃ to adjust the pH to 9˜10, and then it was extracted with DCM (100 mL*3), and the combined organic layers were dried over Na₂SO₄, filtered and concentrated under reduced pressure to give a residue. The residue was purified by prep-HPLC (column: Waters Xbridge 150*25 mm*5 um; mobile phase: [water (ammonia hydroxide v/v)-ACN]; gradient: 38%-68% B over 10 min) and the eluent was concentrated to remove MeCN and then lyophilized. I-169 (124.76 mg, 108.98 μmol, 23.87% yield, 99.354% purity) was obtained as a white solid. LCMS (Method D): Rt=0.367 min, (M+H)=1137.5. SCF: Retention time: 3.353 min. ¹H NMR (400 MHz, METHANOL-d4) δ=8.37 (d, J=7.8 Hz, 1H), 7.95 (d, J=6.2 Hz, 1H), 7.91-7.80 (m, 2H), 7.78-7.68 (m, 2H), 7.52-7.41 (m, 3H), 7.40-7.33 (m, 1H), 7.20-7.10 (m, 2H), 6.55-6.43 (m, 2H), 4.49-4.49 (m, 1H), 4.56-4.48 (m, 1H), 4.39 (s, 2H), 4.33-4.26 (m, 1H), 4.06 (dd, J=6.8, 14.0 Hz, 2H), 3.82-3.64 (m, 11H), 3.57-3.46 (m, 4H), 3.24-3.06 (m, 4H), 2.80-2.63 (m, 4H), 2.59-2.39 (m, 8H), 2.28-2.17 (m, 2H), 2.05-1.98 (m, 1H), 1.92-1.74 (m, 10H), 1.72-1.65 (m, 2H), 1.47-1.39 (m, 3H), 1.31-1.05 (m, 8H). ¹⁹F NMR (400 MHz, CHLOROFORM-d) δ=−120.807.

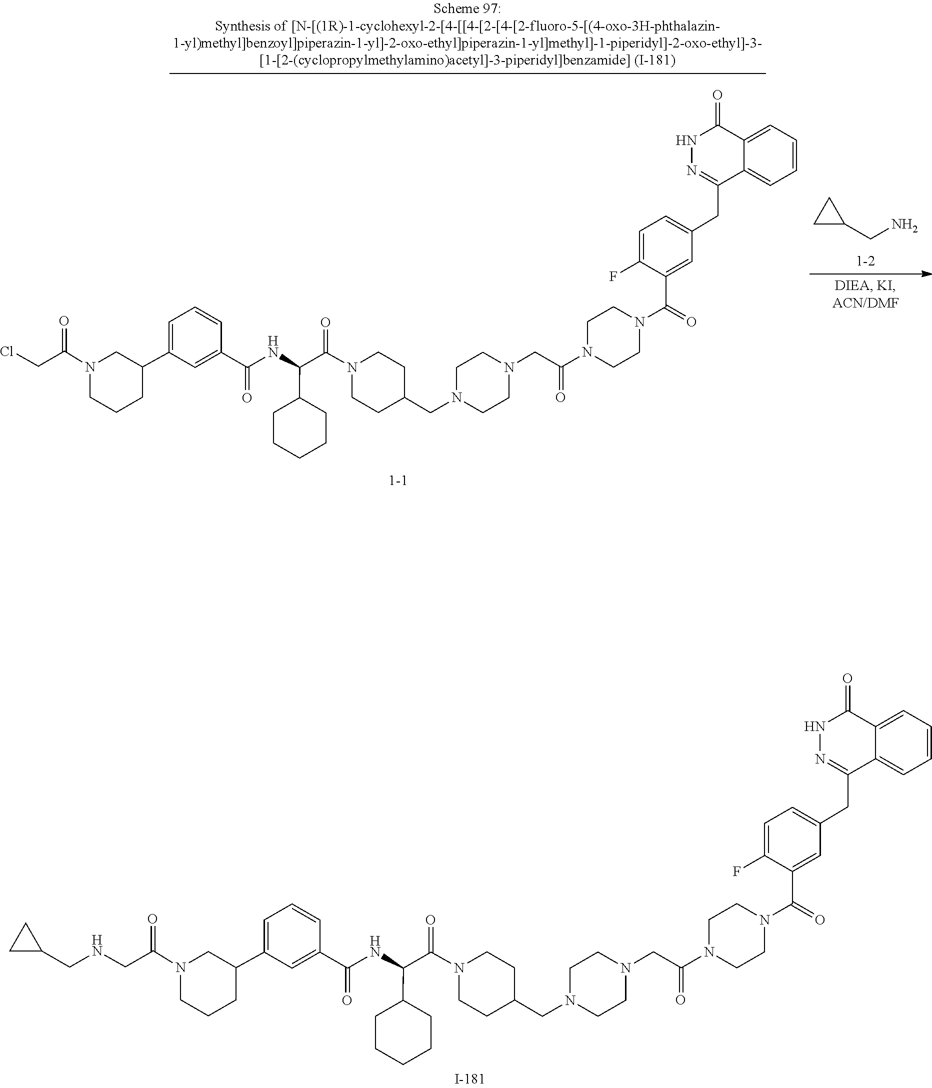

To a solution of intermediate 1-2 (3.22 mg, 45.33 μmol, 1.5 eq) in ACN (0.2 mL) and DMF (0.2 mL) was added DIEA (15.62 mg, 120.89 μmol, 21.06 μL, 4 eq) under N₂ and the mixture was stirred at 25° C. for 0.5 h, then intermediate 1-1 (30 mg, 30.22 μmol, 1 eq) and KI (2.51 mg, 15.11 μmol, 0.5 eq) were added to the mixture and it was stirred at 60° C. for 2 hr. The reaction mixture was concentrated to give a residue. The residue was purified by reversed-phase HPLC (0.1% FA condition) and lyophilized to give I-181 (13 mg, 12.02 μmol, 39.76% yield, 99.223% purity, FA salt) as a yellow gum. LCMS (Method D): Retention time: 0.360 min, (M+1)=1027.6. SFC: Retention time: 4.517 min, 5.886 min. ¹H NMR (400 MHz, CHLOROFORM-d) δ=10.38-9.87 (m, 1H), 8.47 (d, J=7.6 Hz, 1H), 8.35 (s, 1H), 7.82-7.66 (m, 5H), 7.44-7.29 (m, 4H), 7.25-7.16 (m, 1H), 7.06 (t, J=8.4 Hz, 1H), 5.16-4.99 (m, 1H), 4.71-4.52 (m, 2H), 4.29 (s, 2H), 4.17-4.06 (m, 1H), 3.99-3.42 (m, 10H), 3.41-2.92 (m, 7H), 2.87-2.62 (m, 5H), 2.61-2.38 (m, 8H), 1.91-1.62 (m, 12H), 1.33-1.00 (m, 9H), 0.64 (d, J=6.8 Hz, 2H), 0.39-0.27 (m, 2H). ¹⁹F NMR (376 MHz, METHANOL-d4) δ=−117.611

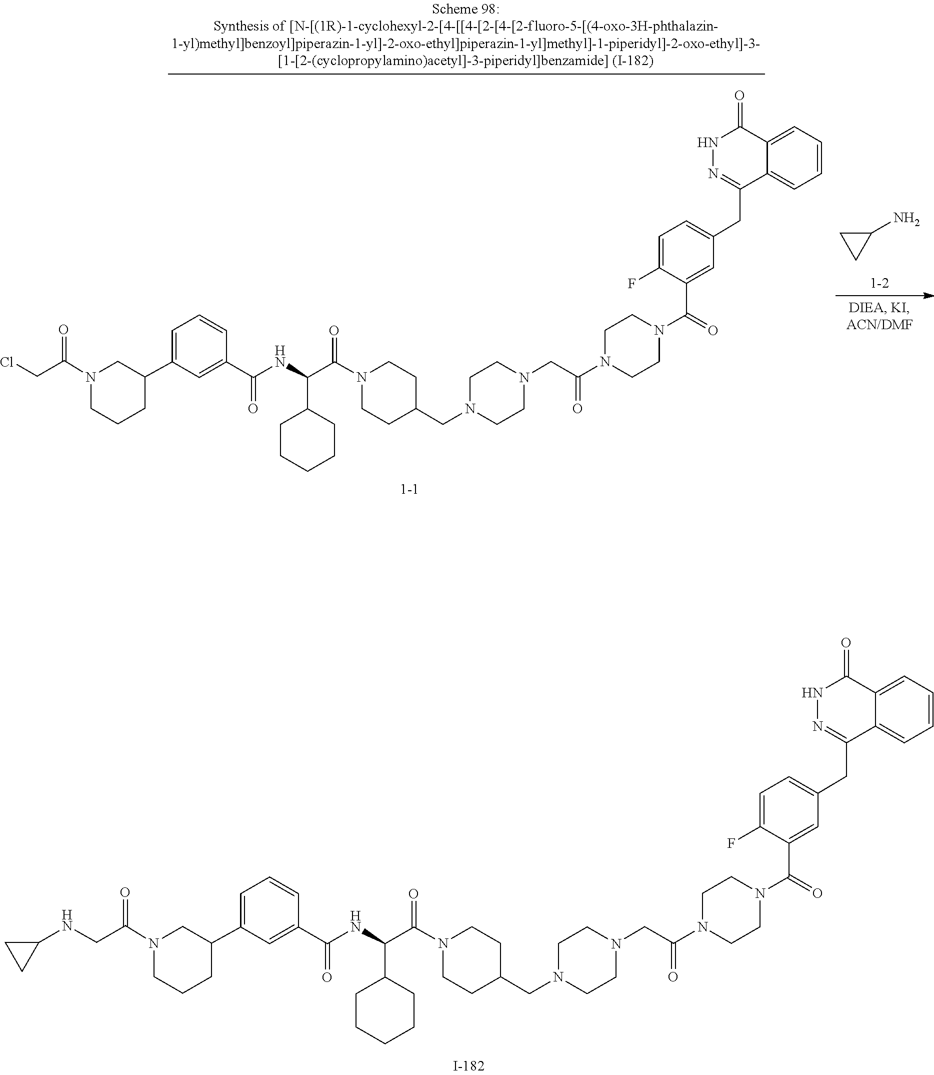

To a solution of intermediate 1-2 (2.59 mg, 45.33 μmol, 3.14 μL, 1.5 eq) in ACN (0.2 mL) and DMF (0.2 mL) was added DIEA (15.62 mg, 120.89 μmol, 21.06 μL, 4 eq) at 25° C. under N₂ and stirred at 25° C. for 0.5 hr, then intermediate 1-1 (30 mg, 30.22 μmol, 1 eq) and KI (2.51 mg, 15.11 μmol, 0.5 eq) were added to the mixture and it was stirred at 60° C. for 2 hr. The reaction mixture was concentrated to give a residue. The residue was purified by reversed-phase HPLC (0.1% FA condition) and lyophilized to give I-182 (10.04 mg, 8.83 μmol, 29.22% yield, 93.179% purity, FA salt) as a white solid. LCMS (Method D): Retention time: 0.353 min, (M+1)=1013.5. SFC: Retention time: 2.667 min, 2.975 min. ¹H NMR (400 MHz, CHLOROFORM-d) δ=10.19-9.97 (m, 1H), 8.47 (d, J=7.2 Hz, 1H), 8.23 (s, 1H), 7.83-7.63 (m, 5H), 7.44-7.30 (m, 4H), 7.15-7.02 (m, 2H), 5.13-5.01 (m, 1H), 4.77-4.66 (m, 1H), 4.65-4.55 (m, 1H), 4.32-4.25 (m, 2H), 4.16-4.06 (m, 1H), 3.85-3.75 (m, 2H), 3.74-3.67 (m, 2H), 3.65-3.51 (m, 4H), 3.41-3.16 (m, 4H), 3.14-3.04 (m, 2H), 2.82-2.62 (m, 3H), 2.61-2.40 (m, 8H), 2.29-2.18 (m, 4H), 1.82-1.72 (m, 8H), 1.51-1.38 (m, 3H), 1.30-1.05 (m, 9H), 0.60-0.38 (m, 4H). ¹⁹F NMR (376 MHz, METHANOL-d4) δ=−117.603

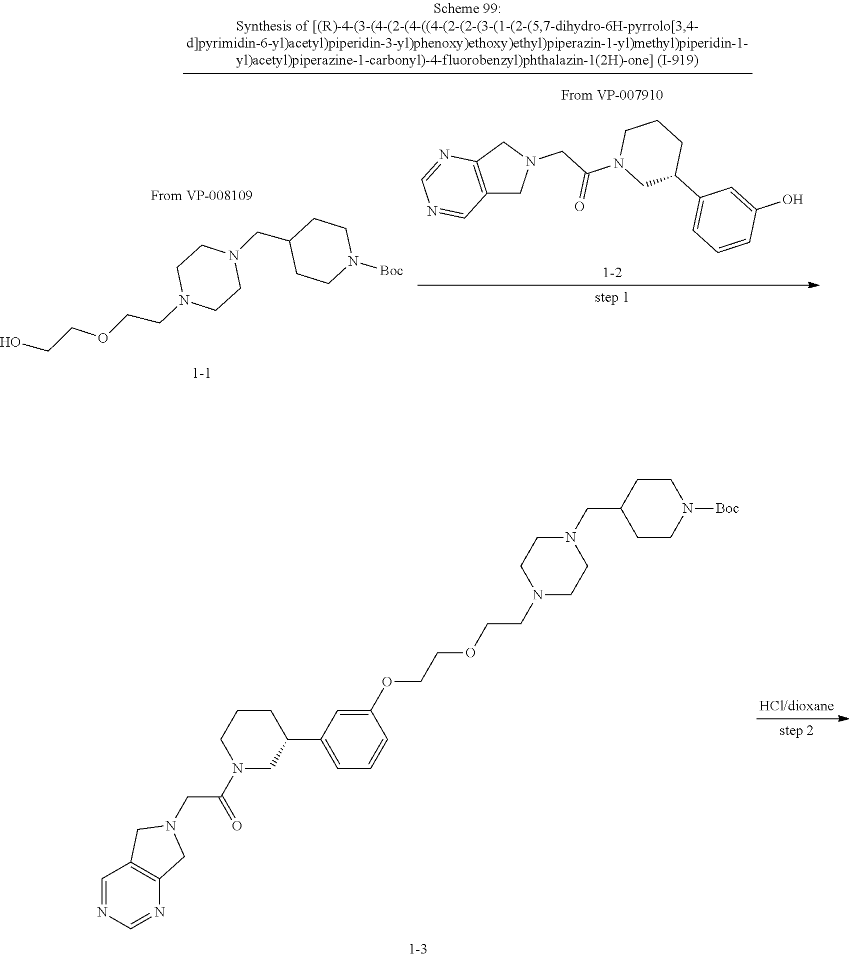 [SMILES: CC(C)(C)OC(=O)N1CCC(CN2CCN(CCOCCO)CC2)CC1 ; O=C(CN1Cc2cncnc2C1)N1CCC[C@H](c2cccc(O)c2)C1 ; CC(C)(C)OC(=O)N1CCC(CN2CCN(CCOCCOc3cccc([C@H]4CCCN(C(=O)CN5Cc6cncnc6C5)C4)c3)CC2)CC1]

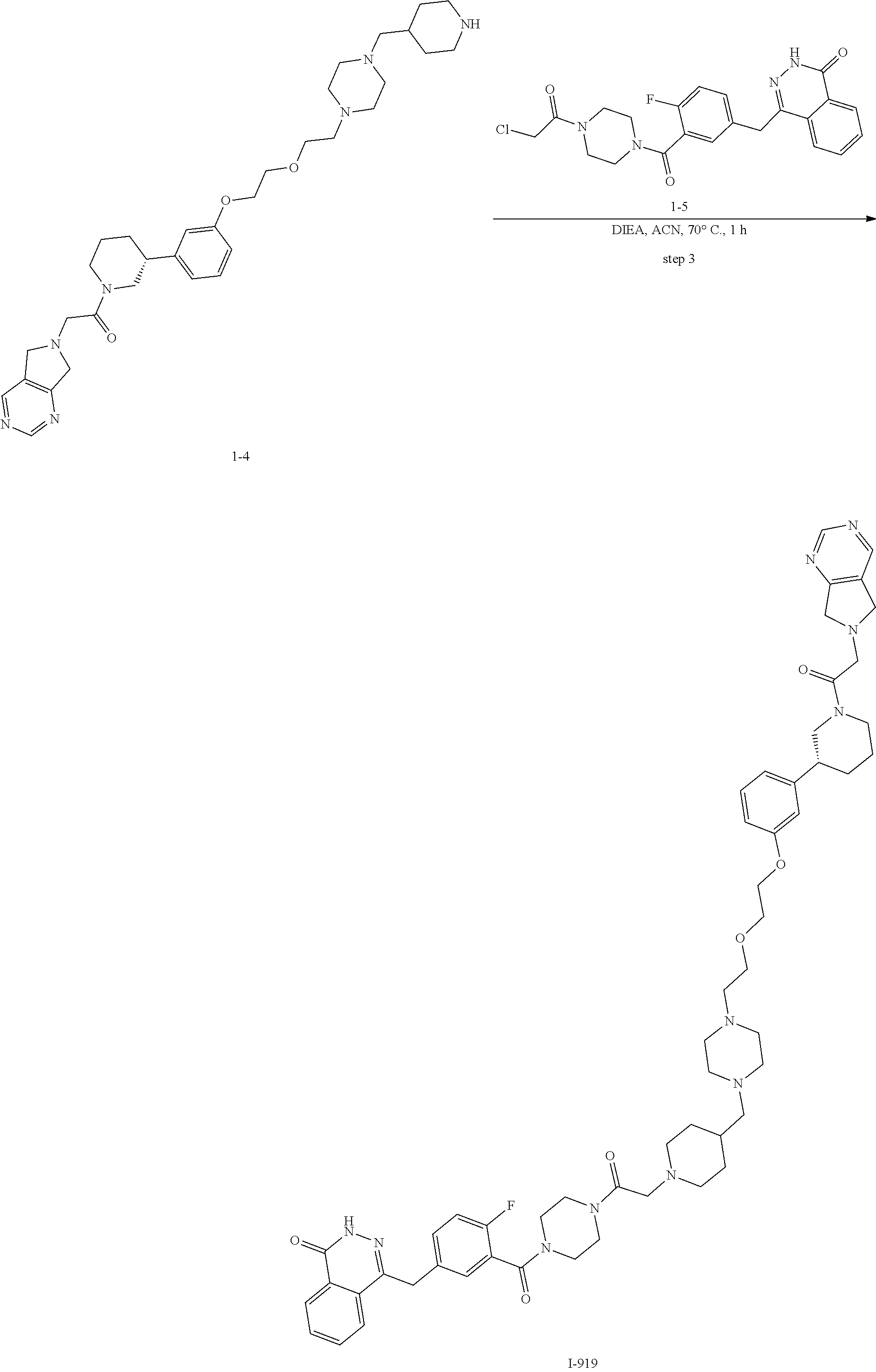 [SMILES: O=C(CN1Cc2cncnc2C1)N1CCC[C@H](c2cccc(OCCOCCN3CCN(CC4CCNCC4)CC3)c2)C1 ; O=C(CCl)N1CCN(C(=O)c2cc(Cc3n[nH]c(=O)c4ccccc34)ccc2F)CC1 ; O=C(CN1CCC(CN2CCN(CCOCCOc3cccc([C@H]4CCCN(C(=O)CN5Cc6cncnc6C5)C4)c3)CC2)CC1)N1CCN(C(=O)c2cc(Cc3n[nH]c(=O)c4ccccc34)ccc2F)CC1]

### Step 1: Synthesis of Intermediate 1-3.

A mixture of Intermediate 1-1 (115.27 mg, 310.28 μmol, 1.5 eq), Intermediate 1-2 (70 mg, 206.85 μmol, 1 eq), and 2-(tributyl-phosphanylidene)acetonitrile (499.25 mg, 2.07 mmol, 10 eq) in toluene (1 mL) was degassed and purged with N₂ 3 times, and then the mixture was stirred at 90° C. for 6 hr under N₂ atmosphere. The reaction mixture was diluted with H₂O (5 mL) and extracted with EA (10 mL*3). The combined organic layers were dried over Na₂SO₄, filtered and concentrated under reduced pressure to give a residue. The residue was purified by prep-HPLC (FA condition), and the eluent was concentrated and lyophilized to give Intermediate 1-3 (60 mg, 68.42 μmol, 33.08% yield, 84.151% purity, FA salt) as a yellow solid. LCMS (Method D): Retention time: 0.288 min, (M+H)=692.4. ¹H NMR (400 MHz, METHANOL-d4) δ=8.99 (d, J=2.8 Hz, 1H), 8.64 (d, J=2.4 Hz, 1H), 8.59-8.43 (m, 1H), 7.27-7.17 (m, 1H), 6.92-6.74 (m, 3H), 4.62-4.51 (m, 1H), 4.24-3.98 (m, 9H), 3.87-3.78 (m, 5H), 3.20-3.10 (m, 1H), 3.04-2.91 (m, 5H), 2.82-2.59 (m, 8H), 2.36-2.29 (m, 2H), 2.07-1.99 (m, 1H), 1.90-1.56 (m, 7H), 1.45 (s, 9H), 1.13-0.97 (m, 2H)

### Step 2: Synthesis of Intermediate 1-4.

To a solution of Intermediate 1-3 (50 mg, 67.76 μmol, 1 eq, FA) in DCM (0.5 mL) was added HCl/dioxane (2 M, 0.5 mL). The mixture was stirred at 25° C. for 1 hr. The reaction mixture was concentrated under reduced pressure to give a residue. The crude product was used in the next step without further purification. Intermediate 1-4 (45 mg, crude, HCl salt) was obtained as a yellow oil. LCMS (Method D): Retention time: 0.203 min, (M+H)=592.3.

### Step 3: Synthesis of I-919.

To a solution of Intermediate 1-4 (45 mg, 71.63 μmol, 1 eq, HCl salt) and Intermediate 1-5 (34.89 mg, 78.79 μmol, 1.1 eq) in ACN (1 mL) was added DIEA (27.77 mg, 214.88 μmol, 37.43 μL, 3 eq). The mixture was stirred at 70° C. for 1 hr. The reaction mixture was diluted with H₂O (2 mL) and extracted with EA (5 mL*3). The combined organic layers were washed with dried over Na₂SO₄, filtered and concentrated under reduced pressure to give a residue. The residue was purified by prep-HPLC (FA condition), and the eluent was concentrated and lyophilized to give I-919 (27.99 mg, 26.40 μmol, 36.86% yield, 98.507% purity, FA salt) as a yellow gum. LCMS (Method D): Retention time: 0.613 min, (M+H)=998.5. SFC: Retention time: 5.161 min. ¹H NMR (400 MHz, METHANOL-d4) δ=8.98 (d, J=2.8 Hz, 1H), 8.63 (d, J=2.4 Hz, 1H), 8.55 (s, 1H), 8.42-8.31 (m, 1H), 7.97-7.92 (m, 1H), 7.90-7.79 (m, 2H), 7.49 (d, J=6.0 Hz, 1H), 7.39-7.33 (m, 1H), 7.25-7.12 (m, 2H), 6.88-6.84 (m, 1H), 6.82-6.77 (m, 2H), 4.60-4.53 (m, 1H), 4.38 (s, 2H), 4.21-4.12 (m, 5H), 4.08-3.99 (m, 2H), 3.89-3.76 (m, 5H), 3.75-3.63 (m, 6H), 3.60-3.47 (m, 2H), 3.24-3.10 (m, 3H), 2.94-2.83 (m, 2H), 2.77-2.67 (m, 2H), 2.64-2.54 (m, 6H), 2.45 (s, 4H), 2.22-2.15 (m, 2H), 2.10-1.97 (m, 3H), 1.93-1.83 (m, 2H), 1.81-1.68 (m, 3H), 1.65-1.46 (m, 2H), 1.29-1.17 (m, 2H). ¹⁹F NMR (376 MHz, METHANOL-d4) δ=−120.69.

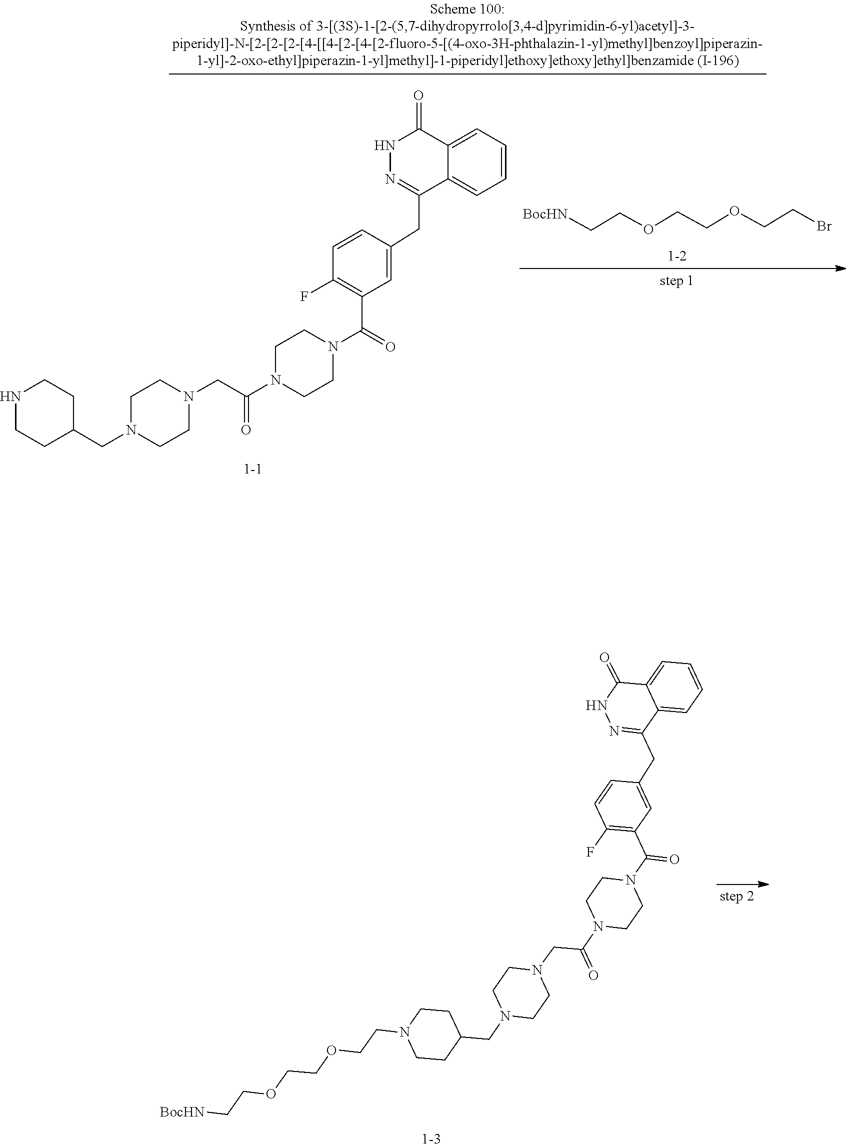 [SMILES: O=C(CN1CCN(CC2CCNCC2)CC1)N1CCN(C(=O)c2cc(Cc3n[nH]c(=O)c4ccccc34)ccc2F)CC1 ; CC(C)(C)OC(=O)NCCOCCOCCBr ; CC(C)(C)OC(=O)NCCOCCOCCN1CCC(CN2CCN(CC(=O)N3CCN(C(=O)c4cc(Cc5n[nH]c(=O)c6ccccc56)ccc4F)CC3)CC2)CC1]

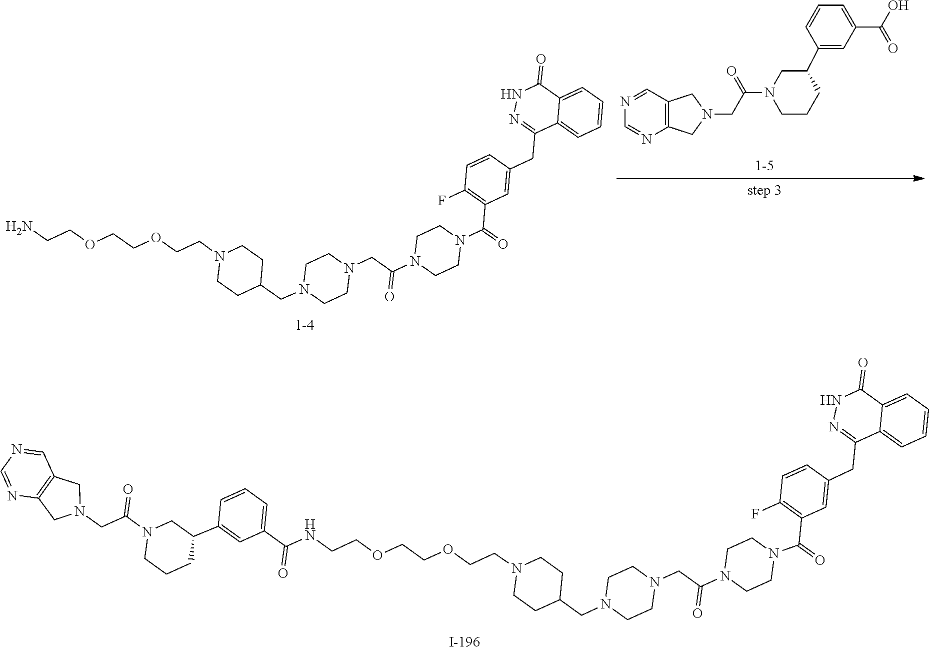

### Step 1: Synthesis of Intermediate 1-3

To a solution of Intermediate 1-1 (400 mg, 638.81 μmol, 1 eq, HCl salt), and Intermediate 1-2 (239.32 mg, 766.57 μmol, 1.2 eq) in ACN (4 mL) was added TEA (193.92 mg, 1.92 mmol, 266.74 μL, 3 eq). The mixture was stirred at 70° C. for 12 hr. The reaction mixture was concentrated under reduced pressure to give a residue, and the residue was poured into water (50 mL) and extracted with EA (20 mL*3). The combined organic layers were dried over Na₂SO₄, filtered and concentrated under reduced pressure to give a residue. The crude product was purified by reversed-phase HPLC (0.1% NH₃·H₂O condition), and the eluent was concentrated to remove ACN and lyophilized to give Intermediate 1-3 as a white solid. LCMS (Method F): Rt=0.640 min, M+H=821.8. ¹H NMR (400 MHz, DMSO-d6) δ=12.59 (s, 1H), 8.26 (d, J=7.6 Hz, 1H), 7.99-7.94 (m, 1H), 7.93-7.78 (m, 2H), 7.49-7.32 (m, 2H), 7.23 (t, J=8.8 Hz, 1H), 6.79-6.68 (m, 1H), 4.33 (s, 2H), 3.65-3.55 (m, 3H), 3.51 (s, 2H), 3.45 (s, 2H), 3.39-3.36 (m, 3H), 3.29 (s, 3H), 3.20-3.11 (m, 3H), 3.10-3.01 (m, 3H), 2.81 (d, J=10.8 Hz, 2H), 2.44-2.22 (m, 9H), 2.06-2.04 (m, 2H), 1.89 (t, J=11.2 Hz, 2H), 1.59 (s, 2H), 1.37 (s, 9H), 1.25-1.13 (m, 2H), 1.11-0.95 (m, 2H).

### Step 2: Synthesis of Intermediate 1-4

A solution of Intermediate 1-3 (130 mg, 158.35 μmol, 1 eq) in HCl/dioxane (2 M, 1.3 mL, 16.42 eq) was stirred at 25° C. for 1 hr. The reaction mixture was concentrated under reduced pressure to give Intermediate 1-4 (119 mg, 157.13 μmol, 99.23% yield, HCl salt) as a white solid. LCMS (Method D): Rt=0.183 min, M+H=721.4.

### Step 3: Synthesis of I-196

To a solution of Intermediate 1-4 (100 mg, 138.72 μmol, 1 eq), and Intermediate 1-5 (50.83 mg, 138.72 μmol, 1 eq) in DMF (1 mL) was added TCFH (58.38 mg, 208.08 μmol, 1.5 eq) and NMI (39.86 mg, 485.52 μmol, 38.70 μL, 3.5 eq). The mixture was stirred at 25° C. for 1 hr. The residue was purified by prep-HPLC (column: Waters Xbridge 150*25 mm*5 um; mobile phase: [water(NH₃H₂O)-ACN]; gradient: 22%-42% B over 14 min). The eluent was lyophilized to give a residue. The residue was further purified by prep-HPLC (column: Phenomenex luna C18 150*25 mm*10 um; mobile phase: [water(FA)-ACN]; gradient: 0%-28% B over 10 min). The eluent was lyophilized to give I-196 (15 mg, 14.03 μmol, 10.11% yield, 100% purity) as a colorless gum. LCMS (Method D): Rt=0.275 min, M+H=1069.5. SFC: Rt=0.657 min. ¹H NMR (400 MHz, METHANOL-d4) δ=9.00 (s, 1H), 8.65 (s, 1H), 8.40-8.37 (m, 2H), 8.00-7.94 (m, 1H), 7.91-9.83 (m, 2H), 7.80-7.67 (m, 2H), 7.51 (s, 1H), 7.48-7.43 (m, 1H), 7.41-7.36 (m, 1H), 7.19 (t, J=8.8 Hz, 1H), 4.65-4.58 (m, 1H), 4.40 (s, 2H), 4.26-4.13 (m, 4H), 4.06 (d, J=12.8 Hz, 1H), 3.92-3.78 (m, 5H), 3.77-3.67 (m, 9H), 3.63-3.51 (m, 6H), 3.48-3.35 (m, 3H), 3.29-3.14 (m, 3H), 2.97 (s, 2H), 2.90-2.77 (m, 2H), 2.76-2.50 (m, 8H), 2.34 (d, J=7.2 Hz, 2H), 2.12-1.95 (m, 3H), 1.89 (t, J=12.8 Hz, 3H), 1.77-1.59 (m, 1H), 1.56-1.23 (m, 3H).

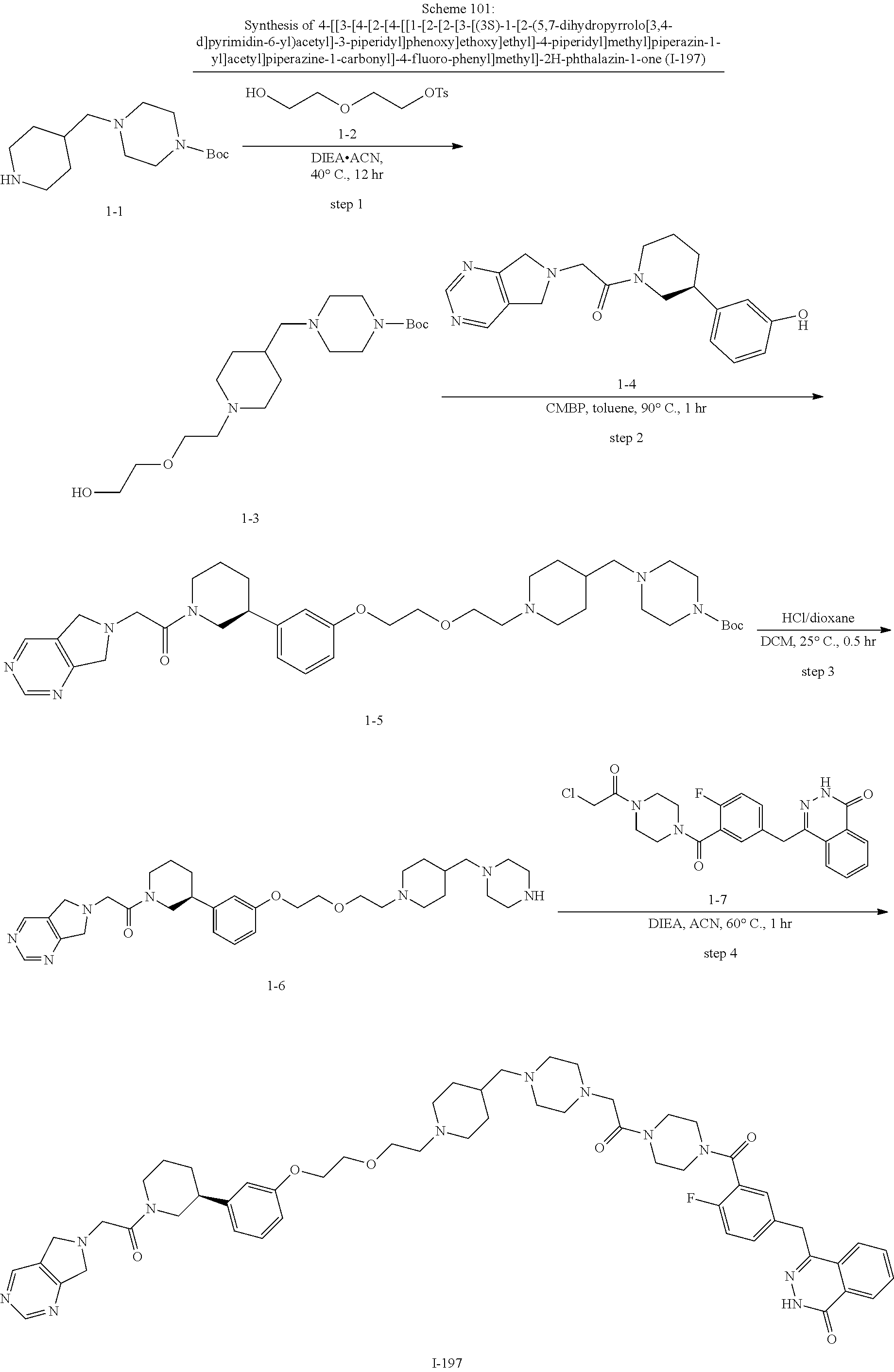

### Step 1: Synthesis of Intermediate 1-3

To a solution of intermediate 1-1 (400 mg, 1.41 mmol, 1 eq) and intermediate 1-2 (367.39 mg, 1.41 mmol, 1 eq) in ACN (4 mL) was added DIEA (547.24 mg, 4.23 mmol, 737.52 μL, 3 eq). The mixture was stirred at 40° C. for 12 hr. The reaction mixture was concentrated in vacuo. The residue was purified by reversed phase HPLC (NH₃·H₂O condition), the eluent was concentrated under reduced pressure to remove ACN and lyophilized to give intermediate 1-3 (460 mg, 1.18 mmol, 83.34% yield, 95% purity) as a yellow oil. LCMS (Method F): Rt=0.545 min, (M+H)=372.2. ¹H NMR (400 MHz, METHANOL-d4) δ=3.68-3.61 (m, 4H), 3.57-3.50 (m, 2H), 3.41 (s, 4H), 3.02 (d, J=11.6 Hz, 2H), 2.62-2.61 (m, 2H), 2.41-2.32 (m, 4H), 2.21 (d, J=7.2 Hz, 2H), 2.10-2.06 (m, 2H), 1.79 (d, J=13.2 Hz, 2H), 1.60-1.56 (m, 1H), 1.45 (s, 9H), 1.32-1.20 (m, 2H).

### Step 2: Synthesis of Intermediate 1-5

A mixture of intermediate 1-3 (164.68 mg, 443.26 μmol, 1.5 eq), intermediate 1-4 (100 mg, 295.51 mol, 1 eq), and CMBP (713.20 mg, 2.96 mmol, 10 eq) in toluene (1 mL) was degassed and purged with N₂ 3 times, and then the mixture was stirred at 90° C. for 12 hr under N₂ atmosphere. The mixture was washed with water (3 mL) and extract with EA (5 mL*3). The combined organic layers were dried over sodium sulfate, filtered and concentrated under reduced pressure to give a residue. The residue was purified by prep-TLC (SiO₂, DCM:MeOH=8:1, Rf=0.4). The excised silica was stirred with DCM:MeOH=8:1 (30 mL), filtered, and then concentrated under reduced pressure to give intermediate 1-5 (160 mg, 224.31 μmol, 75.91% yield, 97% purity) as a white solid. LCMS (Method D): Rt=0.253 min, (M+H)=692.5. SFC: Rt=1.478 min. Step 3: Synthesis of Intermediate 1-6

To a solution of intermediate 1-5 (140 mg, 202.34 μmol, 1 eq) in DCM (1.5 mL) was added HCl/dioxane (2 M, 1.5 mL, 14.83 eq). The mixture was stirred at 25° C. for 0.5 hr. The reaction mixture was concentrated in vacuo. Intermediate 1-6 (140 mg, crude, HCl salt) was obtained as yellow gum and it was used in the next step without further purification. LCMS (Method D): Rt=0.190 min, (M+H)=592.3.

### Step 4: Synthesis of I-197

To a solution of intermediate 1-6 (80 mg, 127.34 μmol, 1 eq, HCl salt) and intermediate 1-7 (56.39 mg, 127.34 μmol, 1 eq) in ACN (0.8 mL) was added DIEA (49.37 mg, 382.02 μmol, 66.54 μL, 3 eq). The mixture was stirred at 60° C. for 1 hr. The reaction mixture was concentrated in vacuo. The residue was purified by prep-HPLC (basic condition), and the eluent was concentrated under reduced pressure to remove ACN and lyophilized to give I-197 (19.35 mg, 18.08 μmol, 14.20% yield, 97.556% purity, FA salt) as yellow gum. LCMS (Method D): Rt=0.262 min, (M+H)=998.4. SFC: Rt=4.916 min. ¹H NMR (400 MHz, METHANOL-d4) δ=8.98 (d, J=5.2 Hz, 1H), 8.64 (d, J=4.4 Hz, 1H), 8.41 (s, 2H), 8.37 (d, J=7.6 Hz, 1H), 8.05-7.76 (m, 3H), 7.50 (s, 1H), 7.37-7.34 (m, 1H), 7.29-7.12 (m, 2H), 6.85 (s, 3H), 4.54 (d, J=11.6 Hz, 1H), 4.38 (s, 2H), 4.32-4.06 (m, 6H), 4.02 (d, J=12.4 Hz, 1H), 3.95-3.71 (m, 8H), 3.68 (s, 2H), 3.63-3.32 (m, 10H), 3.20-3.13 (m, 1H), 2.99-2.96 (m, 2H), 2.84-2.45 (m, 10H), 2.39-2.28 (m, 2H), 2.05-1.93 (m, 3H), 1.92-1.76 (m, 3H), 1.70-1.55 (m, 1H), 1.53-1.36 (m, 2H). ¹⁹F NMR (376 MHz, MeOD-d6) δ=120.778.

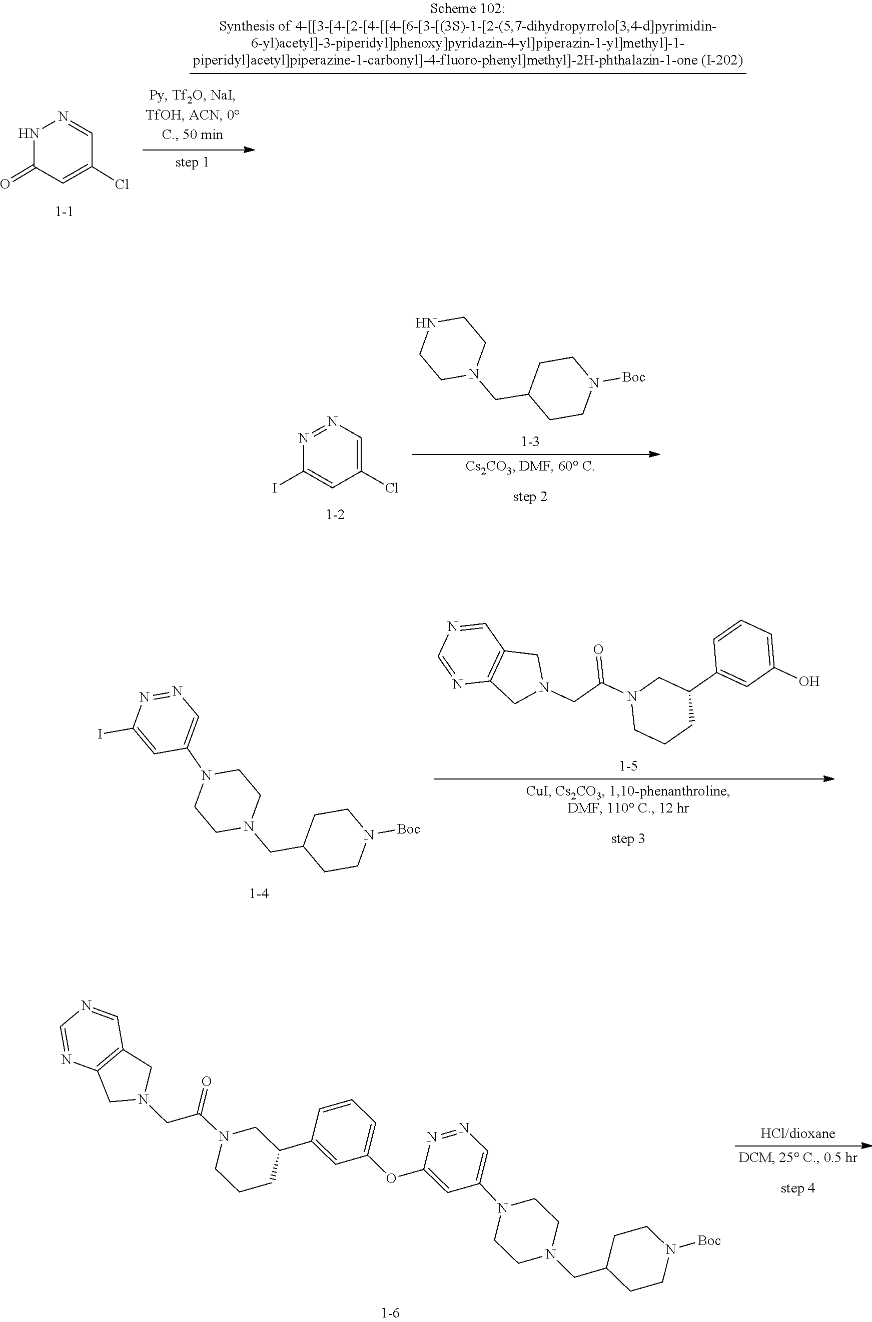 [SMILES: O=c1cc(Cl)cn[nH]1 ; CC(C)(C)OC(=O)N1CCC(CN2CCNCC2)CC1 ; Clc1cnnc(I)c1 ; CC(C)(C)OC(=O)N1CCC(CN2CCN(c3cnnc(I)c3)CC2)CC1 ; O=C(CN1Cc2cncnc2C1)N1CCC[C@@H](c2cccc(O)c2)C1 ; CC(C)(C)OC(=O)N1CCC(CN2CCN(c3cnnc(Oc4cccc([C@@H]5CCCN(C(=O)CN6Cc7cncnc7C6)C5)c4)c3)CC2)CC1]

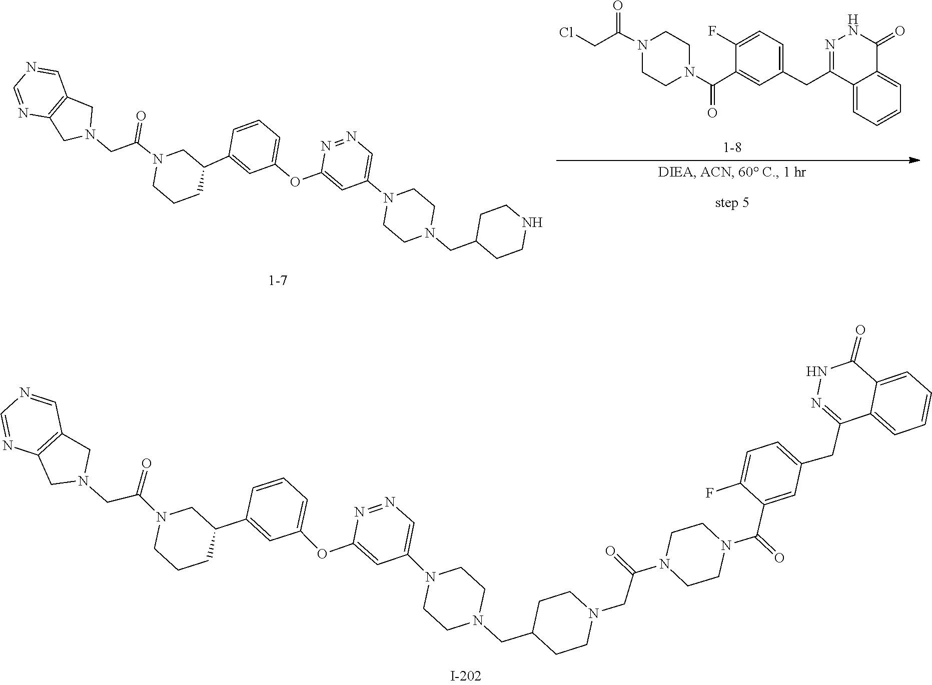 [SMILES: O=C(CN1Cc2cncnc2C1)N1CCC[C@@H](c2cccc(Oc3cc(N4CCN(CC5CCNCC5)CC4)cnn3)c2)C1 ; O=C(CCl)N1CCN(C(=O)c2cc(Cc3n[nH]c(=O)c4ccccc34)ccc2F)CC1 ; O=C(CN1CCC(CN2CCN(c3cnnc(Oc4cccc([C@@H]5CCCN(C(=O)CN6Cc7cncnc7C6)C5)c4)c3)CC2)CC1)N1CCN(C(=O)c2cc(Cc3n[nH]c(=O)c4ccccc34)ccc2F)CC1]

### Step 1: Synthesis of Intermediate 1-2

A solution of pyridine (3.64 g, 45.97 mmol, 3.71 mL, 1.2 eq), intermediate 1-1 (5 g, 38.30 mmol, 1 eq) in ACN (50 mL) was cooled to ⁰° C. and Tf₂O (9.19 g, 32.56 mmol, 5.37 mL, 0.85 eq) was added dropwise. The mixture was stirred for 30 min, and then charged with NaI (5.74 g, 38.30 mmol, 1 eq) in one portion. TfOH (6.32 g, 42.14 mmol, 3.73 mL, 1.1 eq) was added dropwise and the mixture was stirred for 20 min at 0° C. The pH of reaction mixture was acidified to 7-8 with aq. NaHCO₃ and washed with aq. Na₂SO₃, and then extracted with EA (50 mL*3). The combined organic layer was dried over Na₂SO₄, filtered and concentrated in vacuo to give a residue. The residue was purified by column chromatography (SiO₂, PE/EA=1/0 to 0/1, Rf=0.4, PE:EA=5:1) to give intermediate 1-2 (3 g, 11.23 mmol, 29.32% yield, 90% purity) as a yellow solid. LCMS (Method A): Rt=0.292 min, (M+H)=240.9.

### Step 2: Synthesis of Intermediate 1-4

To a solution of intermediate 1-3 (200 mg, 705.70 μmol, 1 eq) and intermediate 1-2 (169.67 mg, 705.70 μmol, 1 eq) in DMF (2 mL) was added Cs₂CO₃ (689.79 mg, 2.12 mmol, 3 eq). The mixture was stirred at 60° C. for 1 hr. The mixture was washed with water (3 mL) and extracted with EA (5 mL*3). The combined organic layers were dried over sodium sulfate, filtered and concentrated under reduced pressure to give a residue. The residue was purified by column chromatography (SiO₂, PE/EA=1/0 to 0/1, Rf=0.35, PE:EA=0:1) to give intermediate 1-4 (310 mg, 616.98 μmol, 87.43% yield, 97% purity) as yellow oil. LCMS (Method D): Rt=0.223 min, (M+H)=488.1. ¹H NMR (400 MHz, METHANOL-d4) δ=8.83 (d, J=2.8 Hz, 1H), 7.37 (d, J=2.8 Hz, 1H), 4.08-4.02 (m, 2H), 3.52-3.46 (m, 4H), 2.77 (s, 2H), 2.57-2.52 (m, 4H), 2.25 (d, J=6.8 Hz, 2H), 1.78 (d, J=10.4 Hz, 3H), 1.45 (s, 9H), 1.12-1.02 (m, 2H).

### Step 3: Synthesis of Intermediate 1-6

To a solution of intermediate 1-4 (120.98 mg, 248.22 μmol, 1.2 eq) and intermediate 1-5 (70 mg, 206.85 μmol, 1 eq) in DMF (1 mL) was added CuI (7.88 mg, 41.37 μmol, 0.2 eq), 1,10-phenanthroline (11.18 mg, 62.06 μmol, 0.3 eq) and Cs₂CO₃ (134.79 mg, 413.71 μmol, 2 eq). The mixture was stirred at 100° C. for 2 hrs. The mixture was washed with water (3 mL) and extracted with EA (5 mL*3). The combined organic layers were dried over sodium sulfate, filtered and concentrated under reduced pressure to give a residue. The residue was purified by prep-TLC (SiO₂, DCM:MeOH=10:1). The excised silica was stirred with DCM:MeOH=10:1 (15 mL) and filtered, then the filtrate was concentrated under reduced pressure to give intermediate 1-6 (90 mg, 122.52 μmol, 59.23% yield, 95% purity) as a yellow gum. LCMS (Method D): Rt=0.288 min, (M+H)=698.4.

### Step 4: Synthesis of Intermediate 1-7

To a solution of intermediate 1-6 (90 mg, 128.96 μmol, 1 eq) in DCM (0.9 mL) was added HCl/dioxane (2 M, 0.9 mL, 13.96 eq). The mixture was stirred at 25° C. for 0.5 hr. The reaction mixture was concentrated in vacuo to give intermediate 1-7 (90 mg, crude, HCl salt) as yellow gum. LCMS (Method D): Rt=0.188 min, (M+H)=598.3.

### Step 5: Synthesis of I-202

To a solution of intermediate 1-7 (90 mg, 141.91 μmol, 1 eq, HCl salt) and intermediate 1-8 (62.85 mg, 141.91 μmol, 1 eq) in ACN (1 mL) was added DIEA (55.02 mg, 425.72 μmol, 74.15 μL, 3 eq). The mixture was stirred at 60° C. for 1 hr. The reaction mixture was concentrated in vacuo to give crude product. The residue was purified by reversed phase HPLC (basic condition), the eluent was concentrated under reduced pressure to remove ACN and lyophilized to give I-202 (15.87 mg, 15.41 μmol, 10.86% yield, 97.532% purity) as an off-white solid. LCMS (Method D): Rt=0.288 min, (M+H)=1004.7. SFC: Rt=1.752 min. ¹H NMR (400 MHz, METHANOL-d4) δ=8.96 (d, J=6.8 Hz, 1H), 8.68 (s, 1H), 8.61 (d, J=14.0 Hz, 1H), 8.37 (d, J=7.6 Hz, 1H), 7.94 (s, 1H), 7.89-7.82 (m, 2H), 7.49 (s, 1H), 7.40-7.34 (m, 2H), 7.20-7.07 (m, 3H), 6.99 (s, 1H), 6.47 (s, 1H), 4.59-4.53 (m, 2H), 4.39 (s, 2H), 4.20-4.10 (m, 4H), 4.07-3.99 (m, 1H), 3.85 (s, 1H), 3.76 (d, J=14.8 Hz, 3H), 3.67-3.51 (m, 4H), 3.48 (s, 4H), 3.27-3.20 (m, 2H), 3.17-3.12 (m, 1H), 2.95-2.88 (m, 2H), 2.80-2.70 (m, 2H), 2.55 (s, 4H), 2.25 (s, 2H), 2.14-1.98 (m, 4H), 1.86-1.75 (m, 4H), 1.64-1.54 (m, 2H), 1.31-1.23 (m, 2H). ¹⁹F NMR (376 MHz, MeOD-d6) δ =120.802.

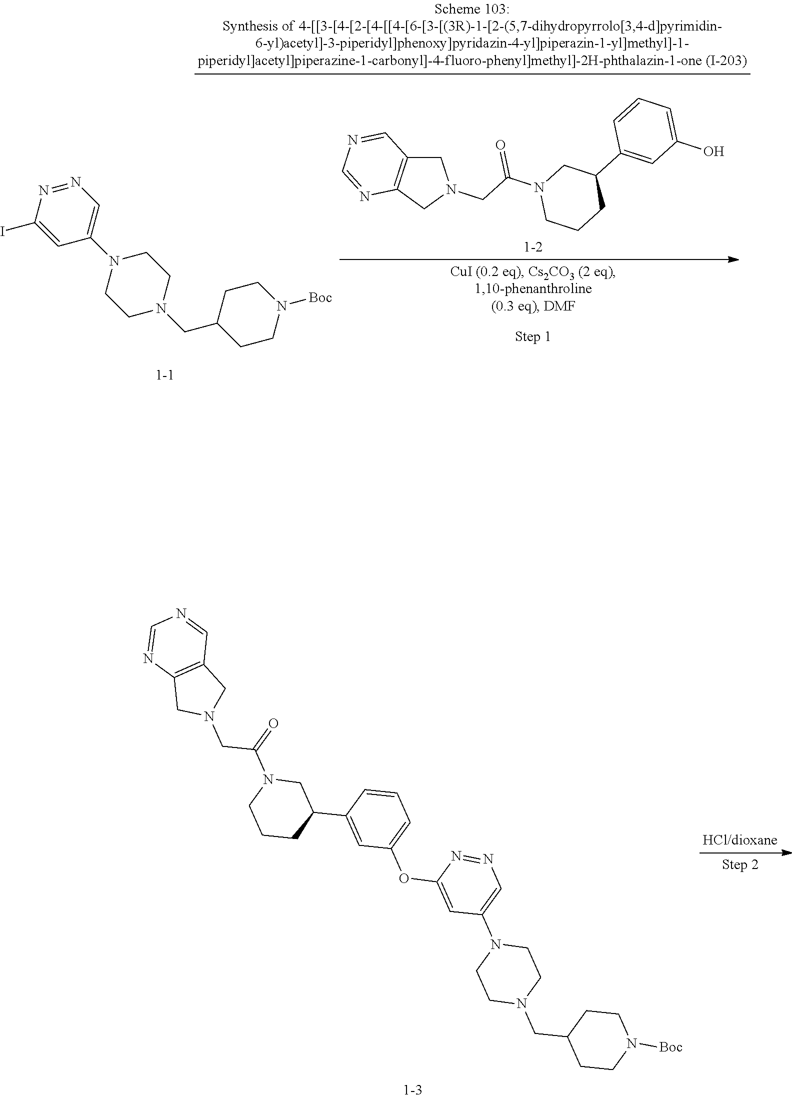 [SMILES: CC(C)(C)OC(=O)N1CCC(CN2CCN(c3cnnc(I)c3)CC2)CC1 ; O=C(CN1Cc2cncnc2C1)N1CCC[C@H](c2cccc(O)c2)C1 ; CC(C)(C)OC(=O)N1CCC(CN2CCN(c3cnnc(Oc4cccc([C@H]5CCCN(C(=O)CN6Cc7cncnc7C6)C5)c4)c3)CC2)CC1]

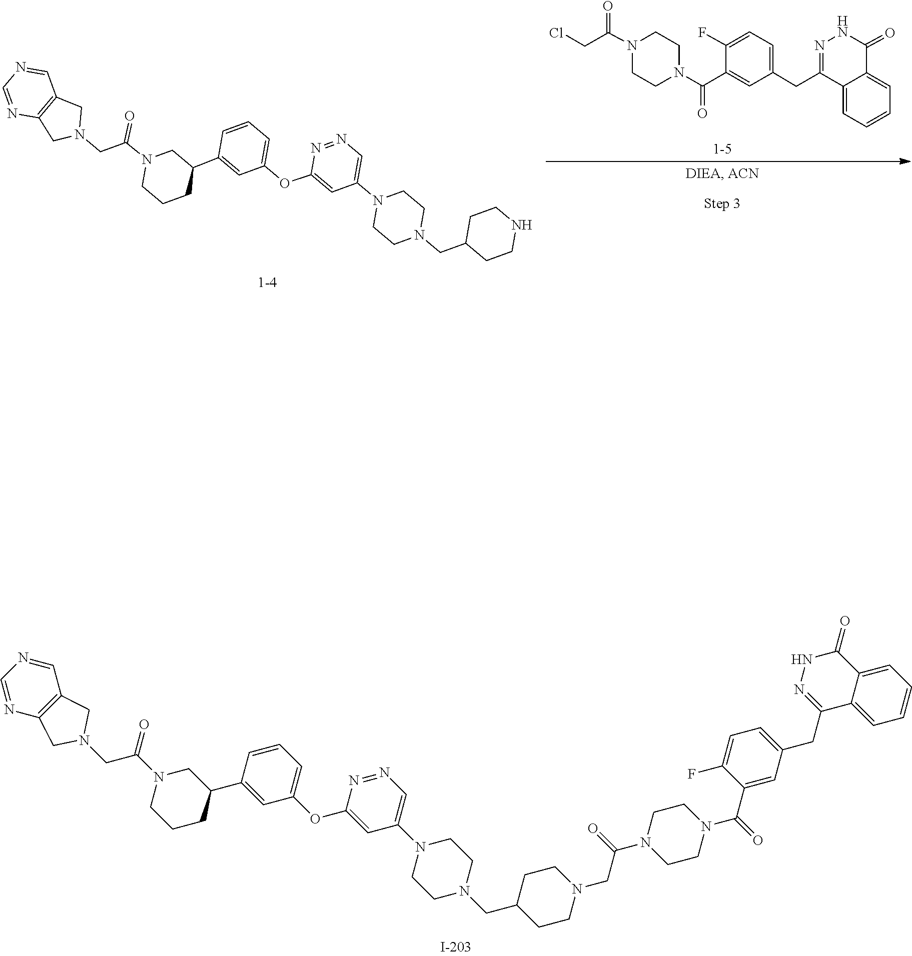 [SMILES: O=C(CN1Cc2cncnc2C1)N1CCC[C@H](c2cccc(Oc3cc(N4CCN(CC5CCNCC5)CC4)cnn3)c2)C1 ; O=C(CCl)N1CCN(C(=O)c2cc(Cc3n[nH]c(=O)c4ccccc34)ccc2F)CC1 ; O=C(CN1CCC(CN2CCN(c3cnnc(Oc4cccc([C@H]5CCCN(C(=O)CN6Cc7cncnc7C6)C5)c4)c3)CC2)CC1)N1CCN(C(=O)c2cc(Cc3n[nH]c(=O)c4ccccc34)ccc2F)CC1]

### Step 1: Synthesis of Intermediate 1-3

To a mixture of 2 intermediate 1-2 (50 mg, 147.75 μmol, 1 eq) and intermediate 1-1 (108.02 mg, 221.63 μmol, 1.5 eq) in DMF (1 mL) was added Cs₂CO₃ (96.28 mg, 295.51 μmol, 2 eq), 1,10-phenanthroline (7.99 mg, 44.33 μmol, 0.3 eq), CuI (5.63 mg, 29.55 μmol, 0.2 eq), and then the mixture was stirred at 100° C. for 1 hour under N₂ atmosphere. The reaction mixture was quenched with water (5 mL) and extracted with EA (2 mL*2). The combined organic layers were concentrated under reduced pressure to give a residue. The residue was purified by prep-TLC (SiO², DCM:MeOH=10:1) to give intermediate 1-3 (80 mg, 97.44 μmol, 65.95% yield, 85% purity) as a yellow solid. LCMS (Method D): Rt=0.297 min, [M+H]⁺=698.5.

### Step 2: Synthesis of Intermediate 1-4

A mixture of intermediate 1-3 (70 mg, 100.31 μmol, 1 eq) in HCl/dioxane (1 mL) was stirred at 20° C. for 10 mins. The reaction mixture was concentrated under reduced pressure to give intermediate 1-4 (70 mg, crude, HCl salt) as a white solid. LCMS (Method D): Rt=0.194 min, [M+H]⁺=598.3.

### Step 3: Synthesis of I-203

To a mixture of intermediate 1-4 (70 mg, 110.37 μmol, 1 eq, HCl salt) and intermediate 1-5 (48.88 mg, 110.37 μmol, 1 eq) in ACN (1 mL) was added DIEA (42.79 mg, 331.12 μmol, 57.67 μL, 3 eq), then the mixture was stirred at 60° C. for 1 hour. The reaction mixture was concentrated under reduced pressure to give a residue. The crude product was purified by reversed-phase HPLC (0.1% FA condition). The eluent was concentrated under reduced pressure to remove MeCN and then lyophilized to give I-203 (9.03 mg, 8.94 μmol, 8.10% yield, 99.38% purity) as a white solid. LCMS (Method D): Rt=0.268 min, [M+H]⁺=1004.3, SFC: Retention time: 2.447 min. ¹H NMR (400 MHz, METHANOL-d4) δ=8.96 (d, J=7.6 Hz, 1H), 8.68 (s, 1H), 8.61 (d, J=14.0 Hz, 1H), 8.37 (d, J=7.2 Hz, 1H), 7.98-7.93 (m, 1H), 7.90-7.81 (m, 2H), 7.49 (s, 1H), 7.39-7.32 (m, 2H), 7.22-7.01 (m, 3H), 7.00-6.97 (m, 1H), 6.47 (s, 1H), 4.59-4.53 (m, 3H), 4.39 (s, 2H), 4.21-4.09 (m, 4H), 4.03-4.01 (m, 1H), 3.95-3.84 (m, 1H), 3.82-3.61 (m, 6H), 3.60-3.41 (m, 7H), 3.28-3.12 (m, 3H), 2.96-2.87 (m, 2H), 2.82-2.69 (m, 2H), 2.55 (s, 4H), 2.31-2.21 (m, 2H), 2.12-2.04 (m, 2H), 1.86-1.77 (m, 3H), 1.59 (d, J=8.4 Hz, 2H), 1.29 (s, 2H). ¹⁹F NMR (376 MHz, DMSO-d6) δ=−120.79.

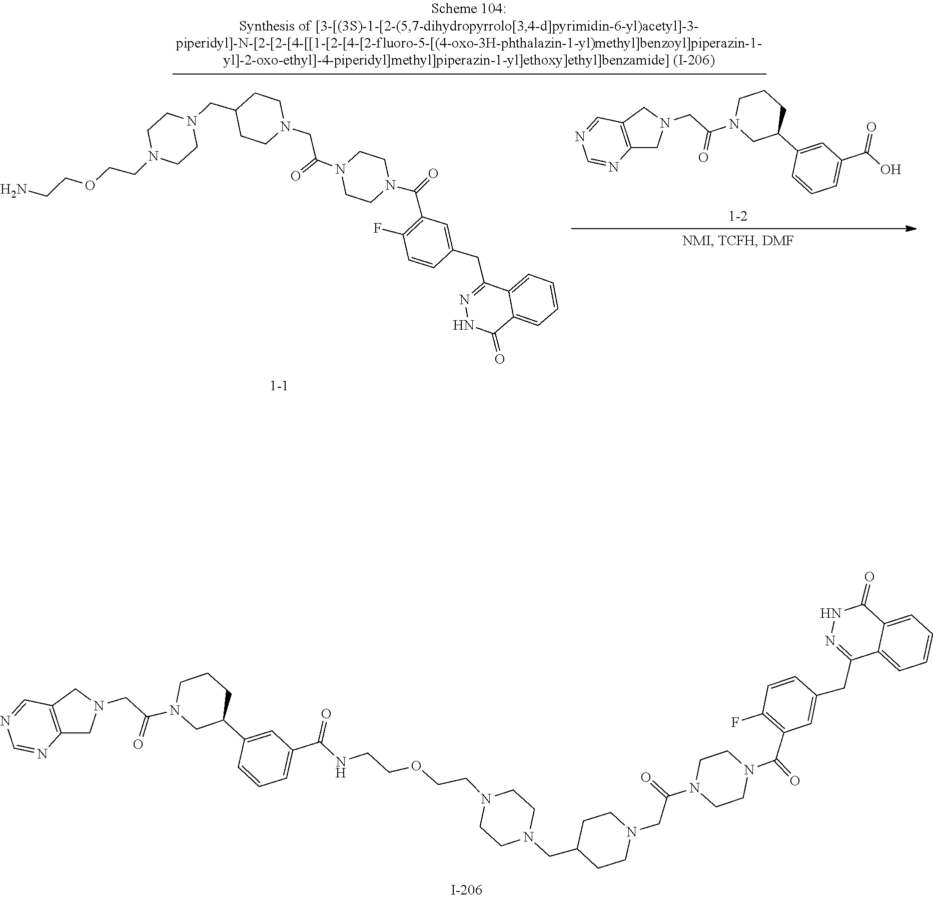 [SMILES: NCCOCCN1CCN(CC2CCN(CC(=O)N3CCN(C(=O)c4cc(Cc5n[nH]c(=O)c6ccccc56)ccc4F)CC3)CC2)CC1 ; O=C(O)c1cccc([C@@H]2CCCN(C(=O)CN3Cc4cncnc4C3)C2)c1 ; O=C(NCCOCCN1CCN(CC2CCN(CC(=O)N3CCN(C(=O)c4cc(Cc5n[nH]c(=O)c6ccccc56)ccc4F)CC3)CC2)CC1)c1cccc([C@@H]2CCCN(C(=O)CN3Cc4cncnc4C3)C2)c1]

To a solution intermediate 1-1 (50 mg, 70.10 μmol, 1 eq, HCl salt) in DMF (1 mL) was added NMI (17.27 mg, 210.30 μmol, 16.76 μL, 3 eq) at 25° C., then to the mixture was added TCFH (29.50 mg, 105.15 μmol, 1.5 eq) and intermediate 1-2 (25.68 mg, 70.10 μmol, 1 eq) at 25° C., and the reaction mixture was stirred at 25° C. for 0.5 hr. The residue was purified by prep-HPLC (column: Waters Xbridge BEH C18 150*25 mm*5 um; mobile phase: [water(NH4HCO3)-ACN]; gradient: 15%-45% B over 10 min) and the eluent was lyophilized to give product. I-206 (15.5 mg, 14.80 μmol, 21.11% yield, 97.885% purity) as a yellow solid. LCMS (Method H): Rt=0.501 min, (M+H)=1025.3. SFC: Rt=0.882 min. ¹H NMR (400 MHz, METHANOL-d4) δ=8.97 (s, 1H), 8.63 (s, 1H), 8.37 (d, J=7.6 Hz, 1H), 7.98-7.92 (m, 1H), 7.90-7.68 (m, 4H), 7.51-7.36 (m, 4H), 7.20-7.12 (m, 1H), 4.60 (d, J=9.6 Hz, 3H), 4.38 (s, 2H), 4.20 (d, J=7.6 Hz, 2H), 4.14 (d, J=10.4 Hz, 2H), 4.05 (d, J=12.8 Hz, 1H), 3.89-3.82 (m, 1H), 3.80-3.70 (m, 4H), 3.68-3.61 (m, 5H), 3.61-3.45 (m, 5H), 3.24 (s, 1H), 3.21-3.13 (m, 2H), 2.96-2.81 (m, 3H), 2.81-2.67 (m, 2H), 2.61-2.58 (m, 2H), 2.54 (d, J=1.2 Hz, 3H), 2.37 (s, 3H), 2.17-2.10 (m, 2H), 2.09-2.00 (m, 3H), 1.91-1.81 (m, 2H), 1.74-1.60 (m, 3H), 1.51-1.42 (m, 1H), 1.29-1.14 (m, 2H), 0.89 (d, J=7.2 Hz, 3H). F¹⁹ NMR (400 MHz, METHAN OL-d4) δ=−120.779.

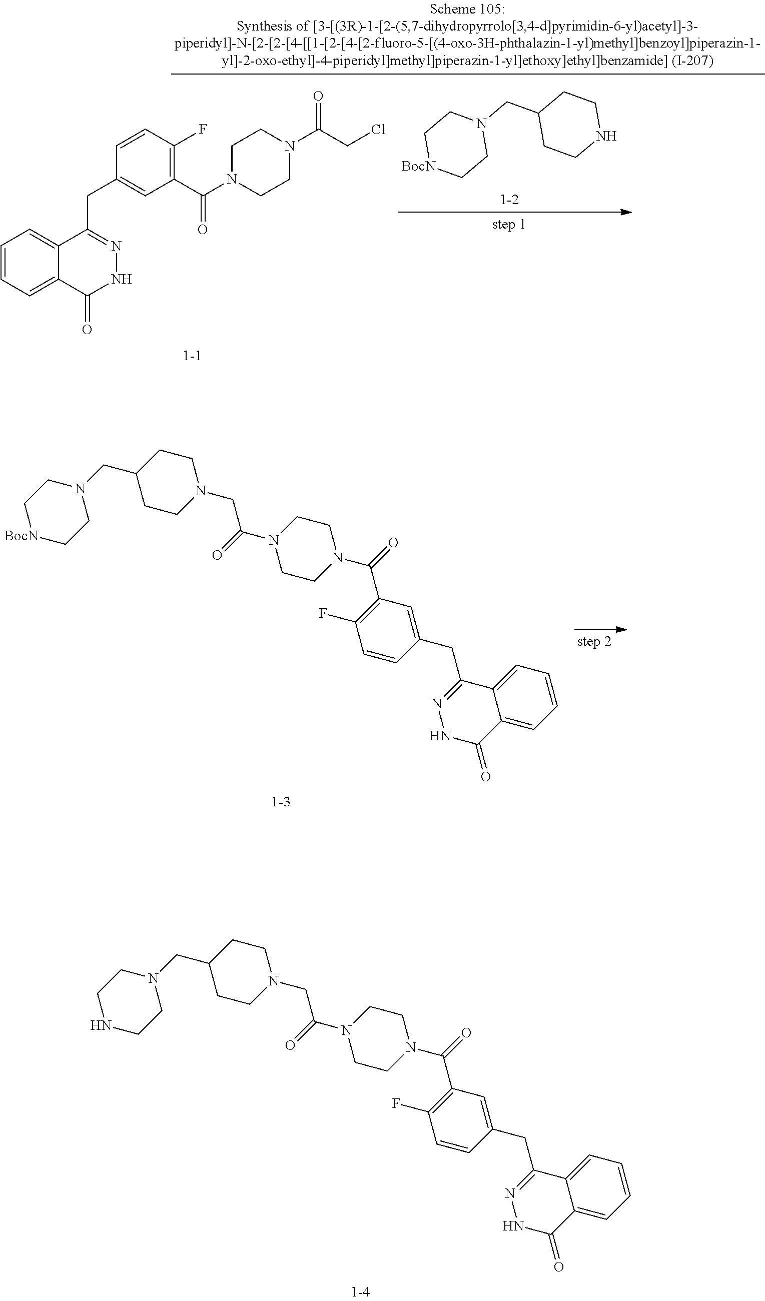 [SMILES: O=C(CCl)N1CCN(C(=O)c2cc(Cc3n[nH]c(=O)c4ccccc34)ccc2F)CC1 ; CC(C)(C)OC(=O)N1CCN(CC2CCNCC2)CC1 ; CC(C)(C)OC(=O)N1CCN(CC2CCN(CC(=O)N3CCN(C(=O)c4cc(Cc5n[nH]c(=O)c6ccccc56)ccc4F)CC3)CC2)CC1 ; O=C(CN1CCC(CN2CCNCC2)CC1)N1CCN(C(=O)c2cc(Cc3n[nH]c(=O)c4ccccc34)ccc2F)CC1]

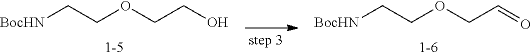 [SMILES: CC(C)(C)OC(=O)NCCOCCO ; CC(C)(C)OC(=O)NCCOCC=O]

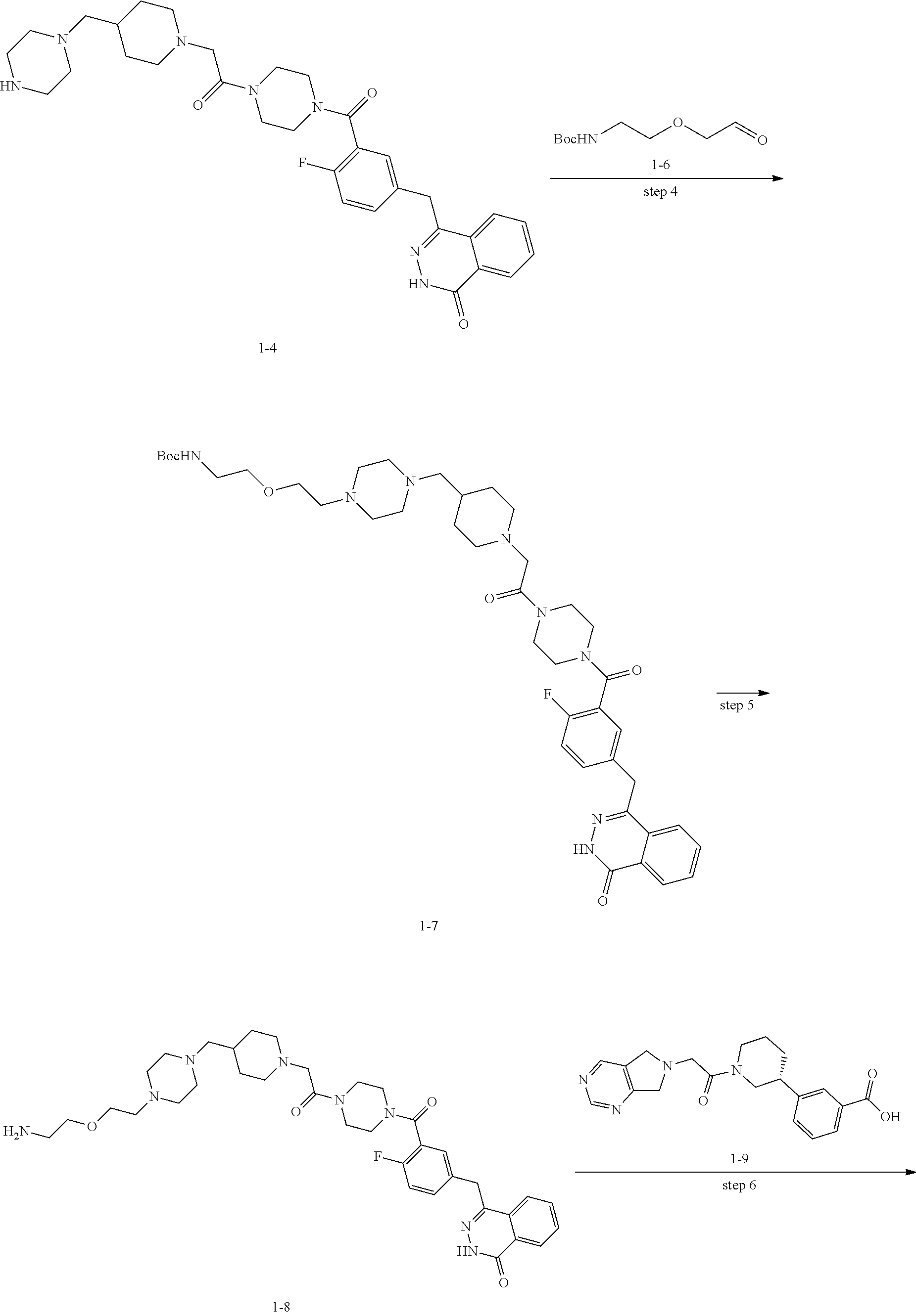 [SMILES: O=C(CN1CCC(CN2CCNCC2)CC1)N1CCN(C(=O)c2cc(Cc3n[nH]c(=O)c4ccccc34)ccc2F)CC1 ; CC(C)(C)OC(=O)NCCOCC=O ; CC(C)(C)OC(=O)NCCOCCN1CCN(CC2CCN(CC(=O)N3CCN(C(=O)c4cc(Cc5n[nH]c(=O)c6ccccc56)ccc4F)CC3)CC2)CC1 ; NCCOCCN1CCN(CC2CCN(CC(=O)N3CCN(C(=O)c4cc(Cc5n[nH]c(=O)c6ccccc56)ccc4F)CC3)CC2)CC1 ; O=C(O)c1cccc([C@H]2CCCN(C(=O)CN3Cc4cncnc4C3)C2)c1]

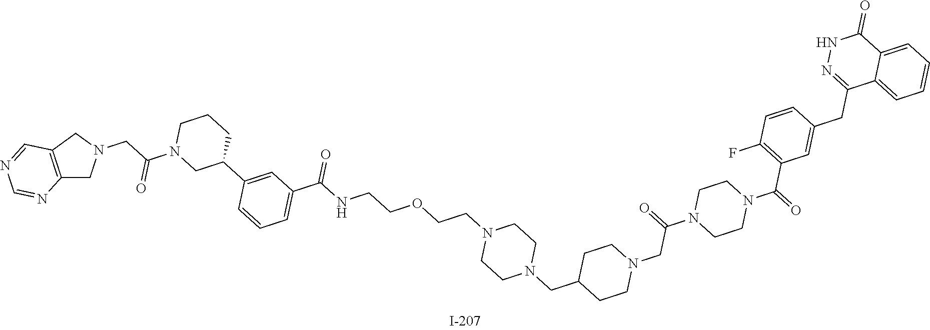 [SMILES: O=C(NCCOCCN1CCN(CC2CCN(CC(=O)N3CCN(C(=O)c4cc(Cc5n[nH]c(=O)c6ccccc56)ccc4F)CC3)CC2)CC1)c1cccc([C@H]2CCCN(C(=O)CN3Cc4cncnc4C3)C2)c1]

### Step 1: Synthesis of Intermediate 1-3

To a solution of intermediate 1-1 (307.17 mg, 1.08 mmol, 1.2 eq) in ACN (4 mL) was added intermediate 1-2 (400 mg, 903.20 μmol, 1 eq) and DIEA (350.20 mg, 2.71 mmol, 471.96 μL, 3 eq). The mixture was stirred at 25° C. for 4 hrs. The mixture was quenched with water (3 mL) and extracted with EA (5 mL×2), dried over Na₂SO₄, filtered and concentrated under vacuum. The residue was purified by column chromatography (SiO₂, DCM/MeOH=1/0 to 0/1, Rf=0.4, DCM:MeOH=10:1) and concentrated in vacuo to give intermediate 1-3 (550 mg, 776.02 μmol, 85.92% yield, 97.33% purity) as a yellow solid. LCMS (Method D): Rt=0.264 min, (M+H)=690.3.

### Step 2: Synthesis of Intermediate 1-4

A mixture of intermediate 1-3 (510 mg, 739.33 μmol, 1 eq) and HCl/dioxane (2 M, 4 mL, 10.82 eq) was stirred at 25° C. for 0.5 hr. The mixture was concentrated in vacuo. The crude product was used in the next step without purification. Intermediate 1-4 (500 mg, crude, HCl salt) was obtained as a yellow solid LCMS (Method D): Rt=0.146 min, (M−55)=590.2.

### Step 3: Synthesis of Intermediate 1-6

To a solution of DMSO (1.90 g, 24.36 mmol, 1.90 mL, 5 eq) in DCM (10 mL) was added dropwise a solution of (COCl)₂ (1.42 g, 11.21 mmol, 980.90 μL, 2.3 eq) in DCM (10 mL) at −78° C. under N₂ atmosphere. Then a solution of intermediate 1-5 (1 g, 4.87 mmol, 1 eq) in DCM (10 mL) was added into the mixture and stirred at −78° C. for 0.5 hr. Then TEA (3.94 g, 38.98 mmol, 5.43 mL, 8 eq) was added into the mixture and it was stirred at −78° C. for 5 hrs. The reaction mixture was poured into water (50 mL) and extracted with EA (20 mL*4). The combined organic layers were dried over Na₂SO₄, filtered and concentrated under reduced pressure to give a residue. The crude product intermediate 1-6 (1 g, crude) was a yellow oil used in the next step without further purification. ¹H NMR (400 MHz, CHLOROFORM-d) δ=5.43-5.34 (m, 1H), 4.15-4.05 (m, 2H), 3.91 (d, J=11.6 Hz, 2H), 3.41-3.23 (m, 4H), 1.46 (s, 9H).

### Step 4: Synthesis of Intermediate 1-7

To a solution of intermediate 1-4 (300 mg, 479.11 μmol, 1 eq, HCl salt) in EtOH (3 mL) was added TEA (193.92 mg, 1.92 mmol, 266.74 μL, 4 eq) and it was stirred at 25° C. for 20 mins. AcOH (172.63 mg, 2.87 mmol, 164.56 μL, 6 eq), and intermediate 1-6 (97.37 mg, 479.11 μmol, 1 eq) were added into the mixture and it was stirred at 25° C. for 20 min. Then the NaBH₃CN (120.43 mg, 1.92 mmol, 4 eq) was added into the mixture. The mixture was at 25° C. for 20 mins. The crude product was purified by reverse phase HPLC (neutral condition) and the eluent was lyophilized to give intermediate 1-7 (200 mg, 240.84 μmol, 50.27% yield, 93.557% purity) as a yellow solid. LCMS (Method D): Rt=0.302 min, (M+H)=777.4.

### Step 5: Synthesis of Intermediate 1-8

A solution of intermediate 1-7 (100 mg, 128.71 μmol, 1 eq) in HCl/dioxane (2 M, 1 mL) was stirred at 25° C. for 0.5 hr. The mixture was concentrated in vacuo. The crude product was used in the next step without purification. Intermediate 1-8 (100 mg, crude, HCl salt) was obtained as a yellow solid. LCMS (Method D): Rt=0.212 min, (M+H)=677.5.

### Step 6: Synthesis of I-207

To a solution intermediate 1-8 (50 mg, 70.10 μmol, 1 eq, HCl salt) in DMF (1 mL) was added NMI (17.27 mg, 210.30 μmol, 16.76 μL, 3 eq) at 25° C., then to the mixture was added TCFH (29.50 mg, 105.15 μmol, 1.5 eq) and intermediate 1-9 (25.68 mg, 70.10 mol, 1 eq) at 25° C., and the reaction mixture was stirred at 25° C. for 0.5 hr. The residue was purified by prep-HPLC (column: Waters Xbridge BEH C18 150*25 mm*5 um; mobile phase: [water(NH₄HCO₃)-ACN]; gradient: 15%-45% B over 10 min) and the eluent was lyophilized to give I-207 (21.1 mg, 20.38 μmol, 29.07% yield, 99.025% purity) as a yellow solid. LCMS (Method H): Rt=0.505 min, (M+H)=1025.3. SFC: Rt=1.440 min. ¹H NMR (400 MHz, METHANOL-d4) δ=8.97 (s, 1H), 8.63 (s, 1H), 8.37 (d, J=7.6 Hz, 1H), 7.99-7.92 (m, 1H), 7.90-7.68 (m, 4H), 7.52-7.34 (m, 4H), 7.20-7.09 (m, 1H), 4.59 (s, 3H), 4.38 (s, 2H), 4.23-4.11 (m, 4H), 4.08-4.01 (m, 1H), 3.89-3.82 (m, 1H), 3.81-3.69 (m, 4H), 3.68-3.60 (m, 5H), 3.60-3.45 (m, 5H), 3.26-3.12 (m, 4H), 2.91-2.82 (m, 2H), 2.81-2.67 (m, 2H), 2.61-2.57 (m, 2H), 2.54 (s, 3H), 2.37 (d, J=0.4 Hz, 3H), 2.17-2.10 (m, 2H), 2.09-1.99 (m, 3H), 1.92-1.82 (m, 2H), 1.73-1.58 (m, 3H), 1.51-1.41 (m, 1H), 1.27-1.11 (m, 2H). ¹⁹F NMR (400 MHz, METHAN OL-d4) δ=−120.695.

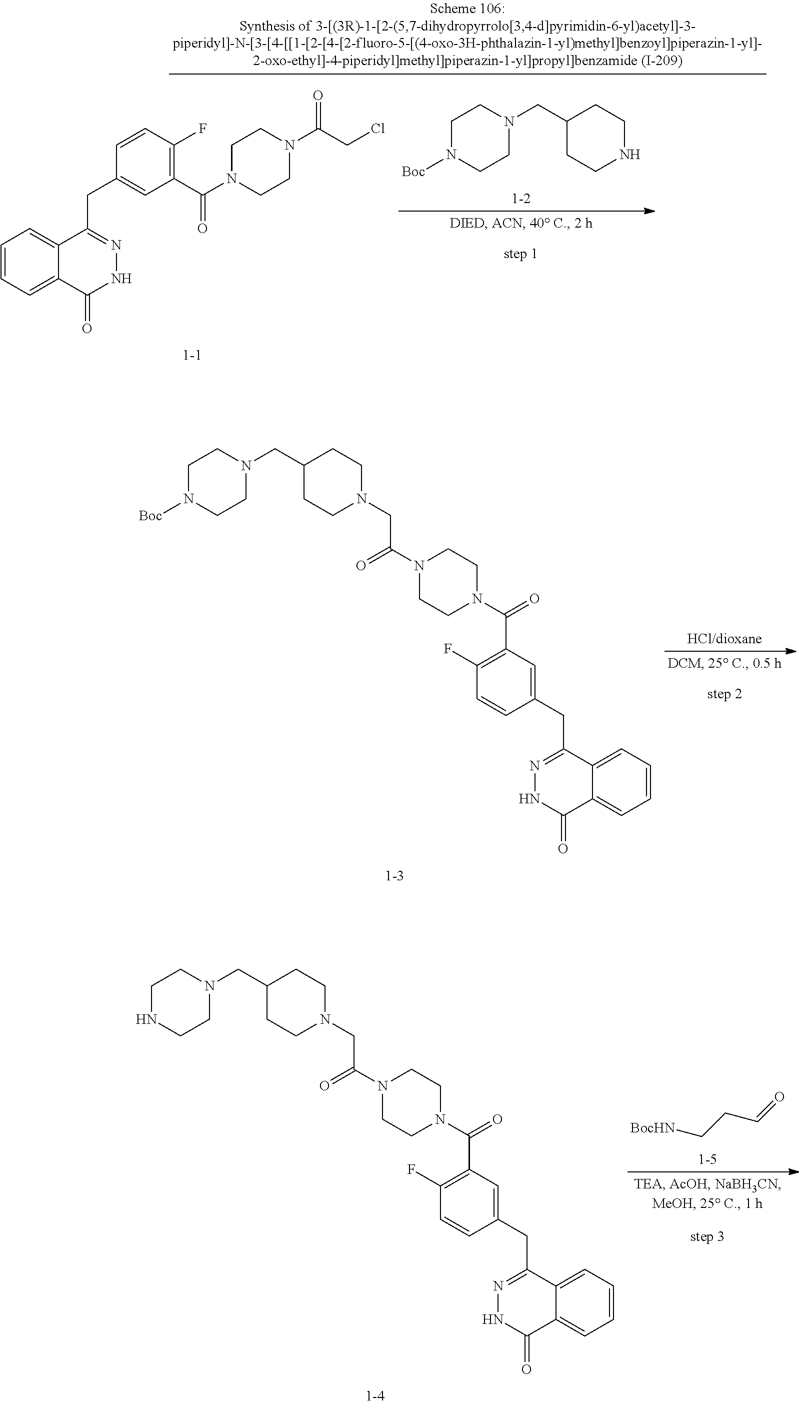 [SMILES: O=C(CCl)N1CCN(C(=O)c2cc(Cc3n[nH]c(=O)c4ccccc34)ccc2F)CC1 ; CC(C)(C)OC(=O)N1CCN(CC2CCNCC2)CC1 ; CC(C)(C)OC(=O)N1CCN(CC2CCN(CC(=O)N3CCN(C(=O)c4cc(Cc5n[nH]c(=O)c6ccccc56)ccc4F)CC3)CC2)CC1 ; O=C(CN1CCC(CN2CCNCC2)CC1)N1CCN(C(=O)c2cc(Cc3n[nH]c(=O)c4ccccc34)ccc2F)CC1 ; CC(C)(C)OC(=O)NCCC=O]

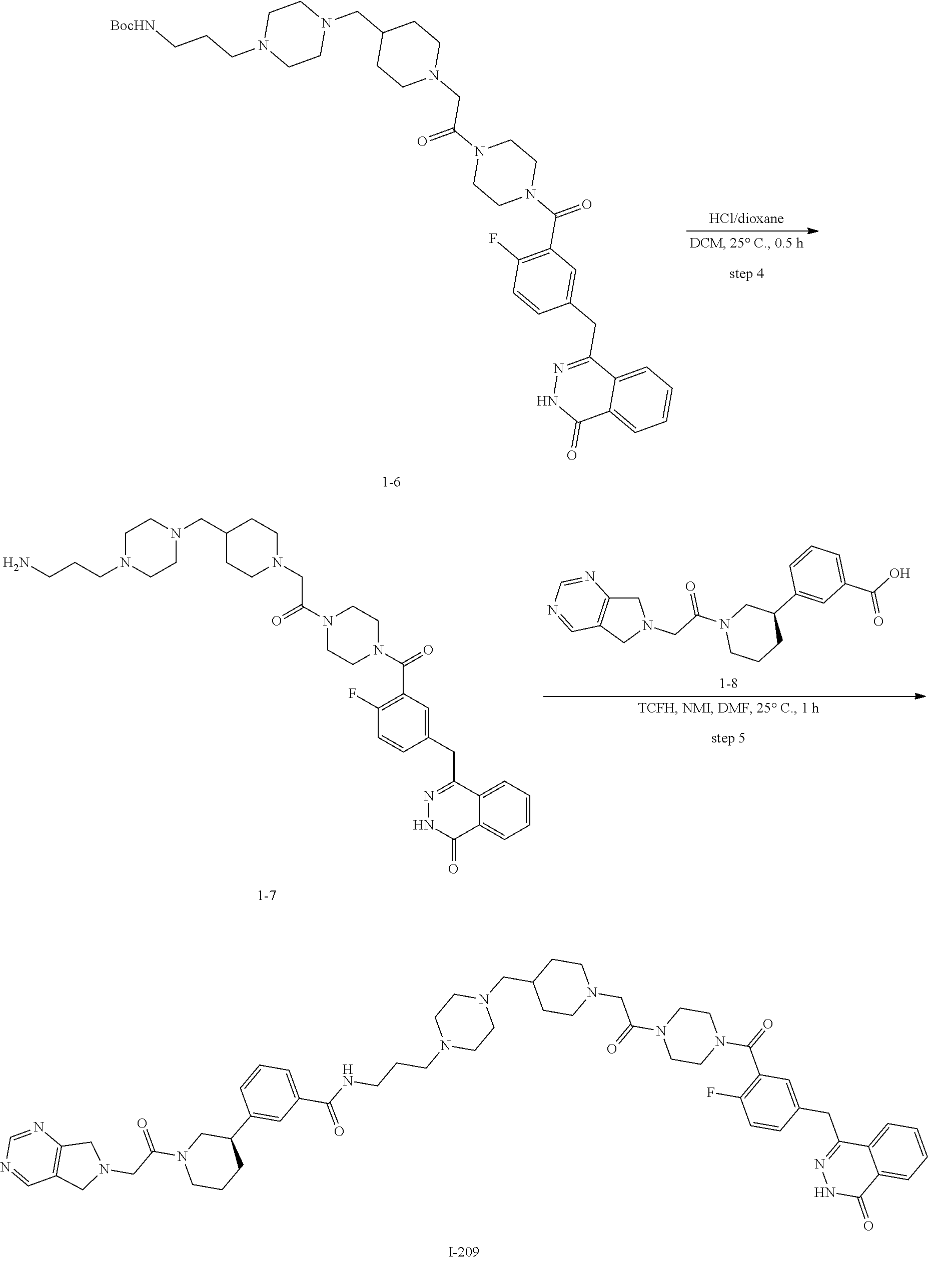 [SMILES: CC(C)(C)OC(=O)NCCCN1CCN(CC2CCN(CC(=O)N3CCN(C(=O)c4cc(Cc5n[nH]c(=O)c6ccccc56)ccc4F)CC3)CC2)CC1 ; NCCCN1CCN(CC2CCN(CC(=O)N3CCN(C(=O)c4cc(Cc5n[nH]c(=O)c6ccccc56)ccc4F)CC3)CC2)CC1 ; O=C(O)c1cccc([C@H]2CCCN(C(=O)CN3Cc4cncnc4C3)C2)c1 ; O=C(NCCCN1CCN(CC2CCN(CC(=O)N3CCN(C(=O)c4cc(Cc5n[nH]c(=O)c6ccccc56)ccc4F)CC3)CC2)CC1)c1cccc([C@H]2CCCN(C(=O)CN3Cc4cncnc4C3)C2)c1]

### Step 1: Synthesis of Intermediate 1-3

To a mixture of intermediate 1-2 (153.58 mg, 541.92 μmol, 1.2 eq) and intermediate 1-1 (200 mg, 451.60 μmol, 1 eq) in ACN (2 mL) was added DIEA (233.46 mg, 1.81 mmol, 314.64 μL, 4 eq). The mixture was stirred at 40° C. for 2 hours. To the reaction mixture was added H₂O (2 mL) and then it was extracted with DCM (5 mL*3), the combined organic phase was washed, dried by Na₂SO₄, filtered and concentrated. The residue was purified by flash silica gel chromatography (ISCO; 12 g SepaFlash Silica Flash Column, Eluent of 0˜60% Ethyl acetate/Methanol gradient @40 mL/min) to give Intermediate 1-3 (310 mg, 440.05 μmol, 97.44% yield, 97.92% purity) as a brown oil. LCMS (Method D): Rt=0.259 min, (M+H)=690.4.

### Step 2: Synthesis of Intermediate 1-4

To a mixture of intermediate 1-3 (260 mg, 369.07 μmol, 1 eq) in DCM (3 mL) was added HCl/dioxane (2 M, 2.60 mL, 14.09 eq). The mixture was stirred at 25° C. for 30 mins. The reaction mixture was concentrated under reduced pressure to give a residue. Intermediate 1-4 (250 mg, crude, HCl salt) as a white solid. LCMS (Method D): Rt=0.228 min, (M+H)=590.2.

### Step 3: Synthesis of Intermediate 1-6

To a mixture of intermediate 1-4 (250 mg, 399.26 μmol, 1 eq, HCl salt) in MeOH (2.5 mL) was added TEA (121.20 mg, 1.20 mmol, 166.71 μL, 3 eq). The mixture was stirred at 25° C. for 15 mins. Then AcOH (143.86 mg, 2.40 mmol, 137.14 μL, 6 eq) and intermediate 1-5 (103.73 mg, 598.89 μmol, 1.5 eq) were added and the mixture was stirred at 25° C. for 15 mins. Then NaBH₃CN (100.36 mg, 1.60 mmol, 4 eq) was added and the mixture was stirred at 25° C. for 1 hr. The reaction mixture was concentrated under reduced pressure to give a residue. The crude product was purified by reversed-phase HPLC (neutral condition) followed by lyophilization to give a residue. Intermediate 1-6 (155 mg, 207.52 μmol, 51.98% yield, 100% purity) was obtained as a white solid. LCMS (Method D): Rt=0.280 min, (M+H)=747.8.

### Step 4: Synthesis of Intermediate 1-7

To a mixture of intermediate 1-6 (100 mg, 133.88 μmol, 1 eq) in DCM (1 mL) was added HCl/dioxane (2 M, 1 mL, 14.94 eq). The mixture was stirred at 25° C. for 30 mins. The reaction mixture was concentrated under reduced pressure to give a residue. Intermediate 1-7 (280 mg, crude, HCl salt) was obtained as a white solid. LCMS (Method D): Rt=0.224 min, (M+H)=647.5.

### Step 5: Synthesis of I-209

To a mixture of intermediate 1-7 (70 mg, 102.45 μmol, 1 eq, HCl salt) and intermediate 1-8 (45.05 mg, 122.94 μmol, 1.2 eq) in ACN (1 mL) was added TCFH (43.12 mg, 153.68 μmol, 1.5 eq) and NMI (29.44 mg, 358.58 μmol, 28.58 μL, 3.5 eq). The mixture was stirred at 25° C. for 1 hour. The mixture was concentrated under reduced pressure to give a residue. The residue was purified by prep-HPLC (column: Waters Xbridge 150*25 mm*5 um; mobile phase: [water (NH₃H2O)-ACN]; gradient: 17%-47% B over 10 min) followed by lyophilization to give I-209 (8 mg, 7.94 μmol, 7.75% yield, 98.835% purity) as a yellow solid. LCMS (Method F): Rt=0.553 min, (M+H)=995.4. SFC: Retention time: 2.756 min. ¹H NMR (400 MHz, METHANOL-d4) δ=8.98 (s, 1H), 8.63 (s, 1H), 8.40-8.34 (m, 1H), 7.99-7.92 (m, 1H), 7.91-7.63 (m, 4H), 7.52-7.35 (m, 4H), 7.16 (t, J=8.8 Hz, 1H), 4.70-4.50 (m, 2H), 4.38 (s, 2H), 4.23-4.11 (m, 4H), 4.05 (d, J=12.4 Hz, 1H), 3.92-3.82 (m, 1H), 3.81-3.64 (m, 5H), 3.60-3.49 (m, 2H), 3.43 (s, 2H), 3.25-3.13 (m, 3H), 2.96-2.80 (m, 3H), 2.80-2.67 (m, 2H), 2.48 (s, 9H), 2.24-2.16 (m, 2H), 2.11-2.01 (m, 3H), 1.93-1.68 (m, 7H), 1.59-1.46 (m, 1H), 1.31-1.14 (m, 2H).

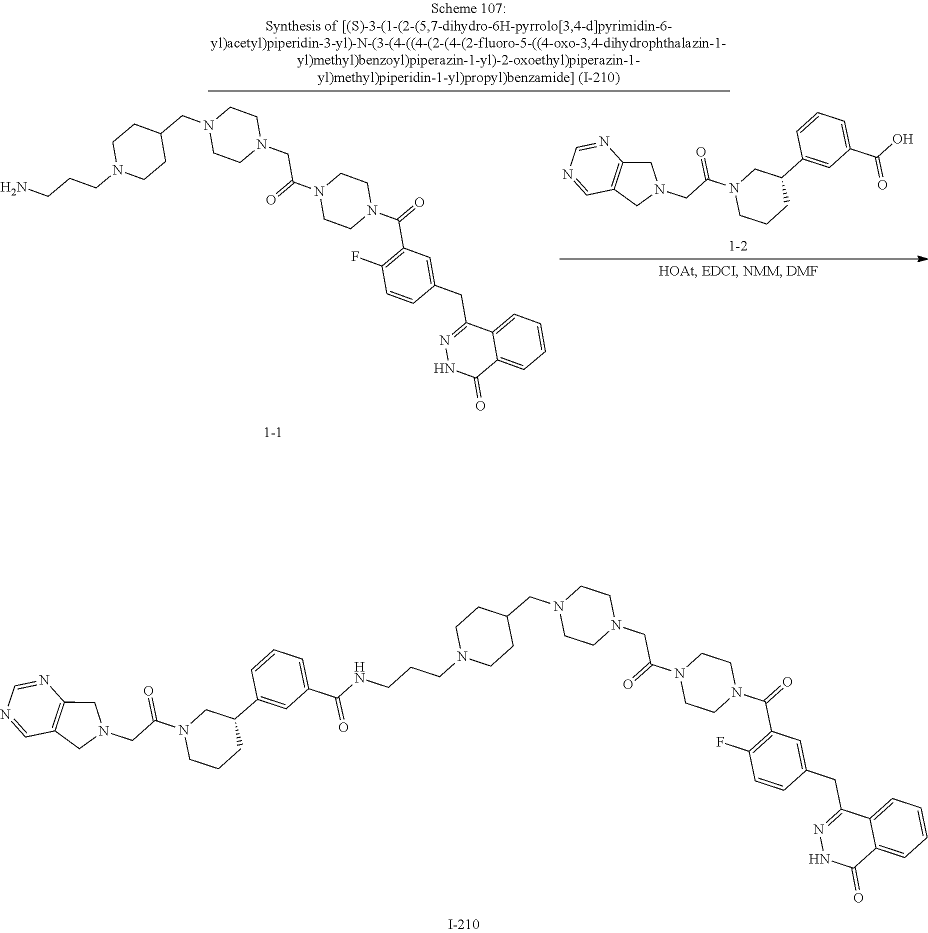 [SMILES: NCCCN1CCC(CN2CCN(CC(=O)N3CCN(C(=O)c4cc(Cc5n[nH]c(=O)c6ccccc56)ccc4F)CC3)CC2)CC1 ; O=C(O)c1cccc([C@@H]2CCCN(C(=O)CN3Cc4cncnc4C3)C2)c1 ; O=C(NCCCN1CCC(CN2CCN(CC(=O)N3CCN(C(=O)c4cc(Cc5n[nH]c(=O)c6ccccc56)ccc4F)CC3)CC2)CC1)c1cccc([C@@H]2CCCN(C(=O)CN3Cc4cncnc4C3)C2)c1]

To a solution of intermediate 1-1 (60 mg, 87.81 μmol, 1 eq, HCl salt) and intermediate 1-2 (38.61 mg, 105.38 μmol, 1.2 eq) in DMF (1 mL) was added TCFH (36.96 mg, 131.72 μmol, 1.5 eq) and NMI (25.23 mg, 307.35 μmol, 24.50 μL, 3.5 eq). The mixture was stirred at 25° C. for 1 hr. To the reaction mixture was added H₂O (3 mL) and then it extracted with DCM (5 mL*3), the combined organic phase was washed, dried by Na₂SO₄, filtered and concentrated. The residue was purified by prep-HPLC (column: Waters Xbridge BEH C18 150*25 mm*5 um; mobile phase: [water (NH₄HCO3)-ACN]; gradient: 15%-45% B over 10 min), the eluent was concentrated to remove ACN and lyophilized to give I-210 (27.16 mg, 25.80 μmol, 29.38% yield, 94.533% purity) as a yellow solid. LCMS (Method H): Retention time: 0.267 min, (M+H)=995.5. SFC: Retention time: 1.096 min. ¹H NMR (400 MHz, METHANOL-d4) δ=8.98 (s, 1H), 8.64 (s, 1H), 8.38-8.36 (m, 1H), 7.95-7.94 (m, 1H), 7.90-7.81 (m, 2H), 7.76-7.64 (m, 2H), 7.52-7.35 (m, 4H), 7.18-7.14 (m, 1H), 4.64-4.55 (m, 2H), 4.39 (s, 2H), 4.23-4.12 (m, 4H), 4.05 (m, 1H), 3.89-3.65 (m, 6H), 3.58-3.47 (m, 2H), 3.42 (s, 2H), 3.26 (s, 2H), 3.23-3.13 (m, 2H), 3.00 (d, J=6.4 Hz, 2H), 2.91-2.38 (m, 12H), 2.22-2.17 (m, 2H), 2.04 (s, 3H), 1.92-1.73 (m, 6H), 1.72-1.45 (m, 2H), 1.33-1.20 (m, 2H).

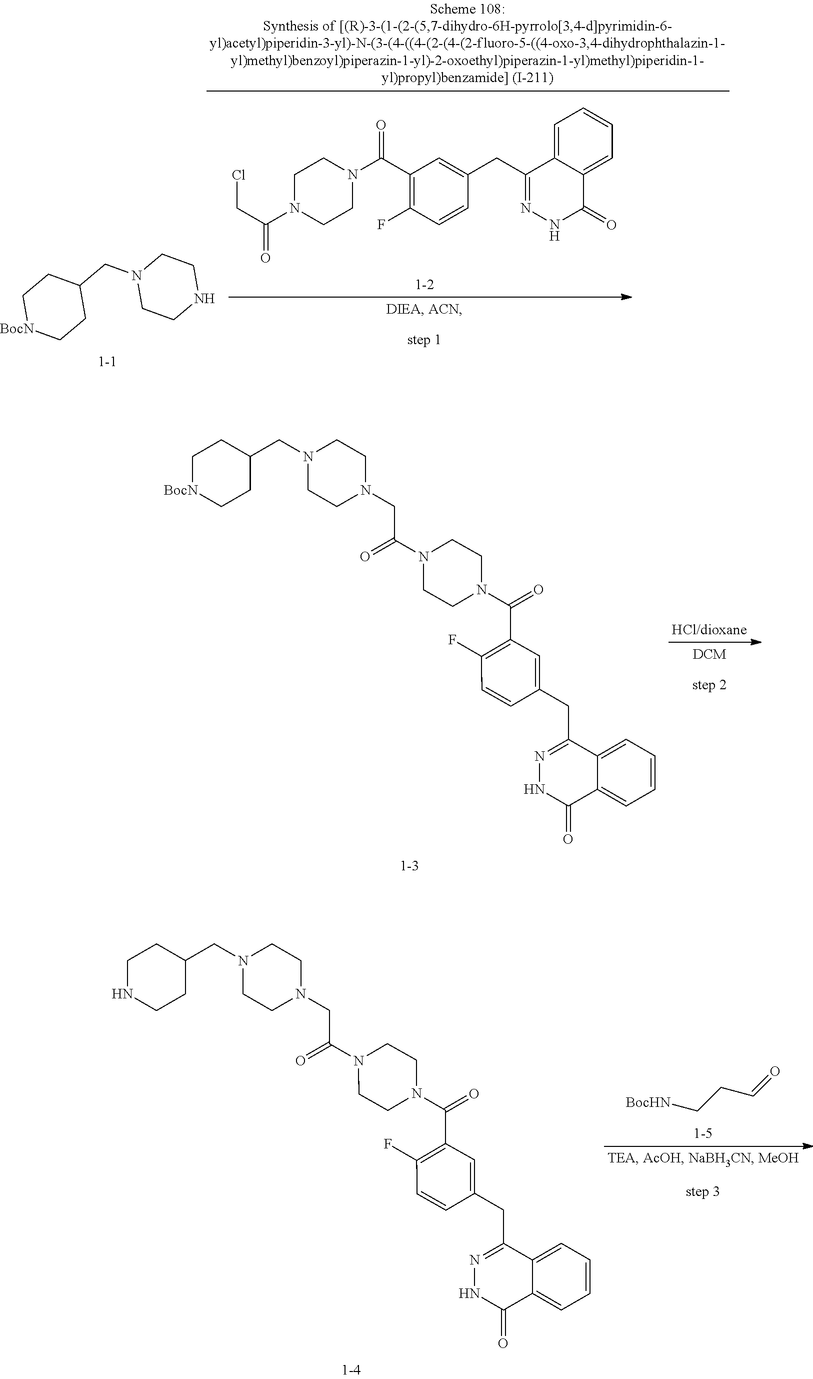 [SMILES: CC(C)(C)OC(=O)N1CCC(CN2CCNCC2)CC1 ; O=C(CCl)N1CCN(C(=O)c2cc(Cc3n[nH]c(=O)c4ccccc34)ccc2F)CC1 ; CC(C)(C)OC(=O)N1CCC(CN2CCN(CC(=O)N3CCN(C(=O)c4cc(Cc5n[nH]c(=O)c6ccccc56)ccc4F)CC3)CC2)CC1 ; O=C(CN1CCN(CC2CCNCC2)CC1)N1CCN(C(=O)c2cc(Cc3n[nH]c(=O)c4ccccc34)ccc2F)CC1 ; CC(C)(C)OC(=O)NCCC=O]

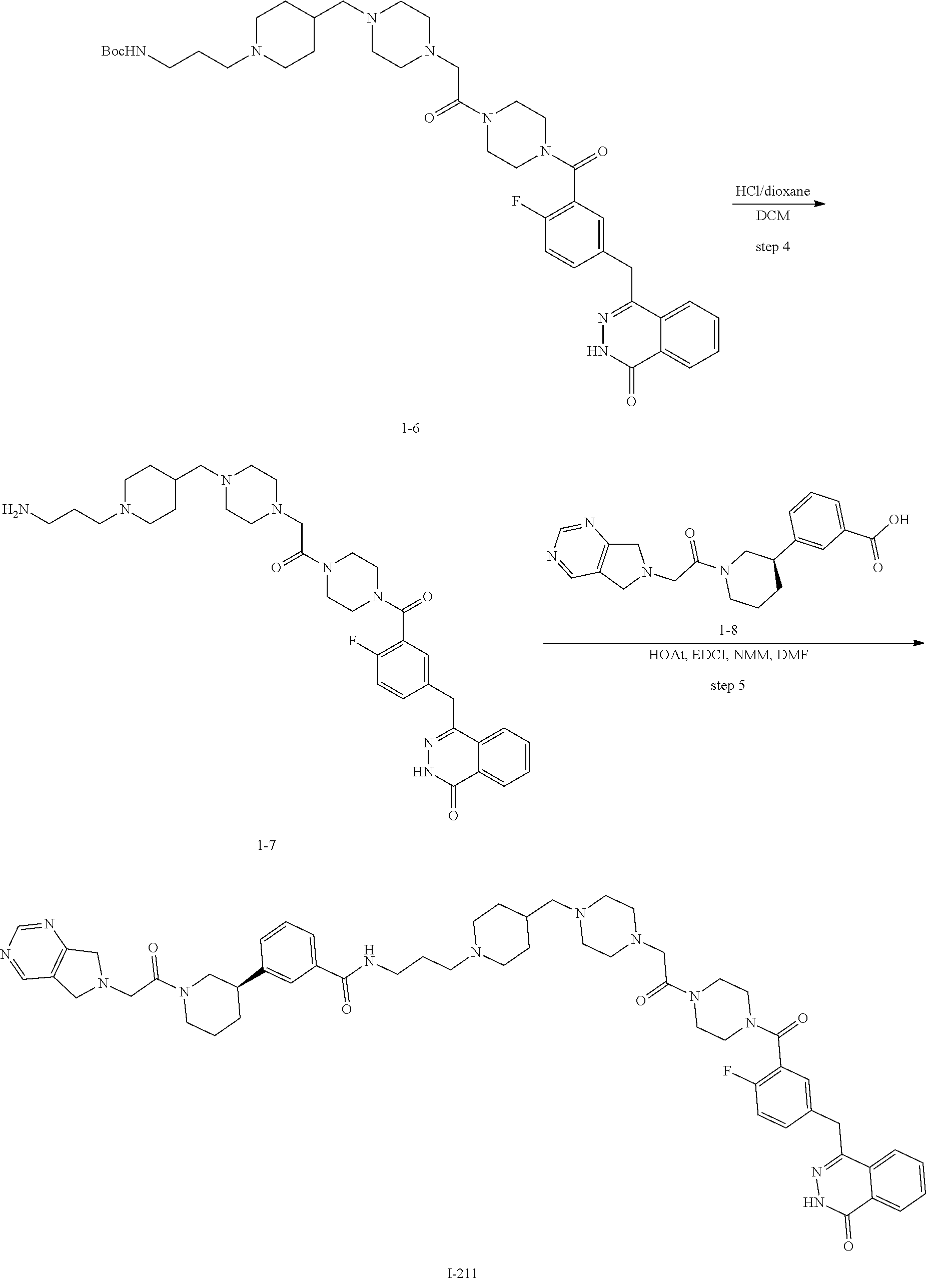 [SMILES: CC(C)(C)OC(=O)NCCCN1CCC(CN2CCN(CC(=O)N3CCN(C(=O)c4cc(Cc5n[nH]c(=O)c6ccccc56)ccc4F)CC3)CC2)CC1 ; NCCCN1CCC(CN2CCN(CC(=O)N3CCN(C(=O)c4cc(Cc5n[nH]c(=O)c6ccccc56)ccc4F)CC3)CC2)CC1 ; O=C(O)c1cccc([C@H]2CCCN(C(=O)CN3Cc4cncnc4C3)C2)c1 ; O=C(NCCCN1CCC(CN2CCN(CC(=O)N3CCN(C(=O)c4cc(Cc5n[nH]c(=O)c6ccccc56)ccc4F)CC3)CC2)CC1)c1cccc([C@H]2CCCN(C(=O)CN3Cc4cncnc4C3)C2)c1]

### Step 1: Synthesis of Intermediate 1-3

To a solution of intermediate 1-1 (200 mg, 705.70 μmol, 1 eq) and intermediate 1-2 (375.04 mg, 846.83 μmol, 1.2 eq) in ACN (2 mL) was added DIEA (364.82 mg, 2.82 mmol, 491.68 μL, 4 eq). The mixture was stirred at 40° C. for 2 hrs. The reaction mixture was concentrated under reduced pressure to give a residue. The residue was purified by column chromatography (SiO₂, Petroleum ether/Ethyl acetate=1/0 to 0/1) and was concentrated to give intermediate 1-3 (500 mg, crude) as a white solid. LCMS (Method D): Retention time: 0.333 min, (M+H)=690.3.

### Step 2: Synthesis of Intermediate 1-4

To a solution of intermediate 1-3 (400 mg, 579.86 μmol, 1 eq) in DCM (3 mL) was added HCl/dioxane (2 M, 2.00 mL, 6.90 eq). The mixture was stirred at 25° C. for 0.5 hr. The reaction mixture was concentrated under reduced pressure to give intermediate 1-4 (400 mg, crude, HCl salt) as a white solid. LCMS (Method D): Retention time: 0.248 min, (M+H)=590.4. ¹H NMR (400 MHz, DMSO-d6) δ=12.65-12.54 (m, 1H), 8.27-8.26 (m, 1H), 7.98-7.81 (m, 3H), 7.51-7.43 (m, 1H), 7.41-7.32 (m, 1H), 7.27-7.2 (m, 1H), 4.34 (s, 2H), 3.69 (s, 2H), 3.45-3.34 (m, 4H), 3.28-3.19 (m, 4H), 3.15-3.08 (m, 2H), 2.84-2.81 (m, 2H), 2.07-1.96 (m, 2H), 1.51-1.36 (m, 2H), 1.31-1.26 (m, 10H), 1.25-1.19 (m, 1H).

### Step 3: Synthesis of Intermediate 1-6

To a solution of intermediate 1-4 (450 mg, 718.66 μmol, 1 eq, HCl salt) and intermediate 1-5 (149.37 mg, 862.40 μmol, 1.2 eq) in MeOH (5 mL) was added TEA (72.72 mg, 718.66 μmol, 100.03 μL, 1 eq) and AcOH (43.16 mg, 718.66 μmol, 41.14 μL, 1 eq). The mixture was stirred at 25° C. for 0.5 hr, then NaBH₃CN (135.49 mg, 2.16 mmol, 3 eq) was added and the mixture was stirred at 25° C. for 0.5 hr. The reaction mixture was concentrated under reduced pressure to give a residue. The residue was purified by prep-HPLC (column: Welch Ultimate XB—SiOH 25*150*10 um; mobile phase: [EtOH+MeOH (4:1, 0.1% NH₄OH)]; gradient: 47%-90% B over 25 min) and the eluent was concentrated to give intermediate 1-6 (250 mg, 334.71 μmol, 46.57% yield) as a white solid. LCMS (Method D): Retention time: 0.283 min, (M+H)=747.2.

### Step 4: Synthesis of Intermediate 1-7

To a solution of intermediate 1-6 (170 mg, 227.60 μmol, 1 eq) in DCM (2 mL) was added HCl/dioxane (2 M, 1.00 mL, 8.79 eq) and the mixture was stirred at 25° C. for 0.5 hr. The reaction mixture was concentrated under reduced pressure to give intermediate 1-7 (200 mg, crude, HCl salt) as a white solid. LCMS (Method D): Retention time: 0.170 min, (M+H)=647.4.

### Step 5: Synthesis of I-211

To a solution of intermediate 1-7 (60 mg, 87.81 μmol, 1 eq, HCl salt) and intermediate 1-8 (38.61 mg, 105.38 μmol, 1.2 eq) in DMF (1 mL) was added TCFH (36.96 mg, 131.72 μmol, 1.5 eq) and NMI (25.23 mg, 307.35 μmol, 24.50 μL, 3.5 eq. The mixture was stirred at 25° C. for 1 hr. To the reaction mixture was added H₂O (3 mL) and then it was extracted with DCM (5 mL*3), the combined organic phase was washed, dried by Na₂SO₄, filtered and concentrated. The residue was purified by prep-HPLC (column: Waters Xbridge BEH C18 150*25 mm*5 um; mobile phase: [water(NH₄HCO₃)-ACN]; gradient: 15%-45% B over 10 min) and the eluent was concentrated to remove ACN and lyophilized to give I-211 (31.17 mg, 29.84 μmol, 33.98% yield, 95.271% purity) as a yellow solid. LCMS (Method H): Retention time: 0.266 min, (M+H)=995.5. SFC: Retention time: 1.902 min. ¹H NMR (400 MHz, METHANOL-d4) δ=8.98 (s, 1H), 8.64 (s, 1H), 8.38-8.36 (m, 1H), 7.94 (d, J=6.0 Hz, 1H), 7.91-7.80 (m, 2H), 7.77-7.62 (m, 2H), 7.54-7.31 (m, 4H), 7.19-7.14 (m, 1H), 4.57 (s, 2H), 4.39 (s, 2H), 4.25-4.11 (m, 4H), 4.09-4.00 (m, 1H), 3.87-3.64 (m, 6H), 3.57-3.48 (m, 2H), 3.43 (d, J=4.8 Hz, 2H), 3.27 (s, 2H), 3.23-3.15 (m, 2H), 3.15-3.04 (m, 2H), 2.85-2.33 (m, 12H), 2.23-2.18 (m, 4H), 2.09-2.02 (m, 1H), 1.97-1.78 (m, 6H), 1.72-1.57 (m, 2H), 1.34-1.23 (m, 2H).

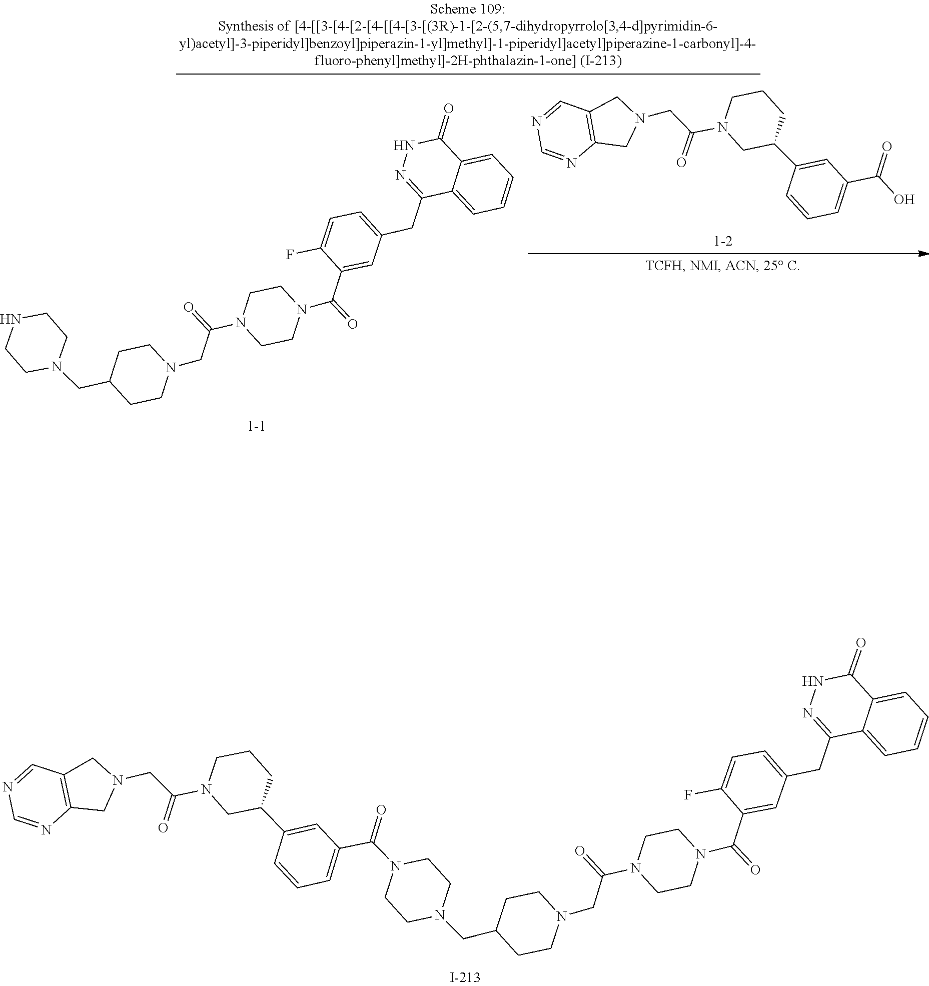

To a mixture of intermediate 1-1 (50 mg, 79.85 μmol, 1 eq, HCl salt) in ACN (0.5 mL) was added intermediate 1-2 (35.11 mg, 95.82 μmol, 1.2 eq), TCFH (26.89 mg, 95.82 μmol, 1.2 eq) and NMI (22.95 mg, 279.48 μmol, 22.28 μL, 3.5 eq) at 25° C., and the mixture was stirred at 25° C. for 1 hr. The reaction mixture was concentrated to give a residue. The residue was purified by prep-HPLC (column: Waters Xbridge 150*25 mm*5 um; mobile phase: [water (NH₃H₂O)-ACN]; gradient: 18%-48% B over 14 min) and lyophilized to give I-213 (21.42 mg, 22.75 μmol, 28.50% yield, 99.654% purity) as a white solid. LCMS (Method F): Retention time: 0.527 min, (M+1)=938.6. SFC: Retention time: 1.869 min. ¹H NMR (400 MHz, METHANOL-d4) δ=9.00 (s, 1H), 8.65 (s, 1H), 8.42-8.35 (m, 1H), 8.00-7.82 (m, 3H), 7.50 (d, J=3.6 Hz, 1H), 7.47-7.24 (m, 5H), 7.18 (t, J=9.2 Hz, 1H), 4.61 (s, 3H), 4.40 (s, 2H), 4.26-4.12 (m, 4H), 4.10-4.00 (m, 1H), 3.92-3.84 (m, 1H), 3.83-3.72 (m, 5H), 3.71-3.66 (m, 1H), 3.58 (s, 1H), 3.53 (s, 1H), 3.49-3.39 (m, 2H), 3.29-3.13 (m, 4H), 2.97-2.87 (m, 2H), 2.87-2.70 (m, 2H), 2.52 (s, 2H), 2.46-2.33 (m, 2H), 2.30-2.20 (m, 2H), 2.14-2.02 (m, 3H), 1.94-1.73 (m, 4H), 1.72-1.51 (m, 2H), 1.34-1.19 (m, 2H).

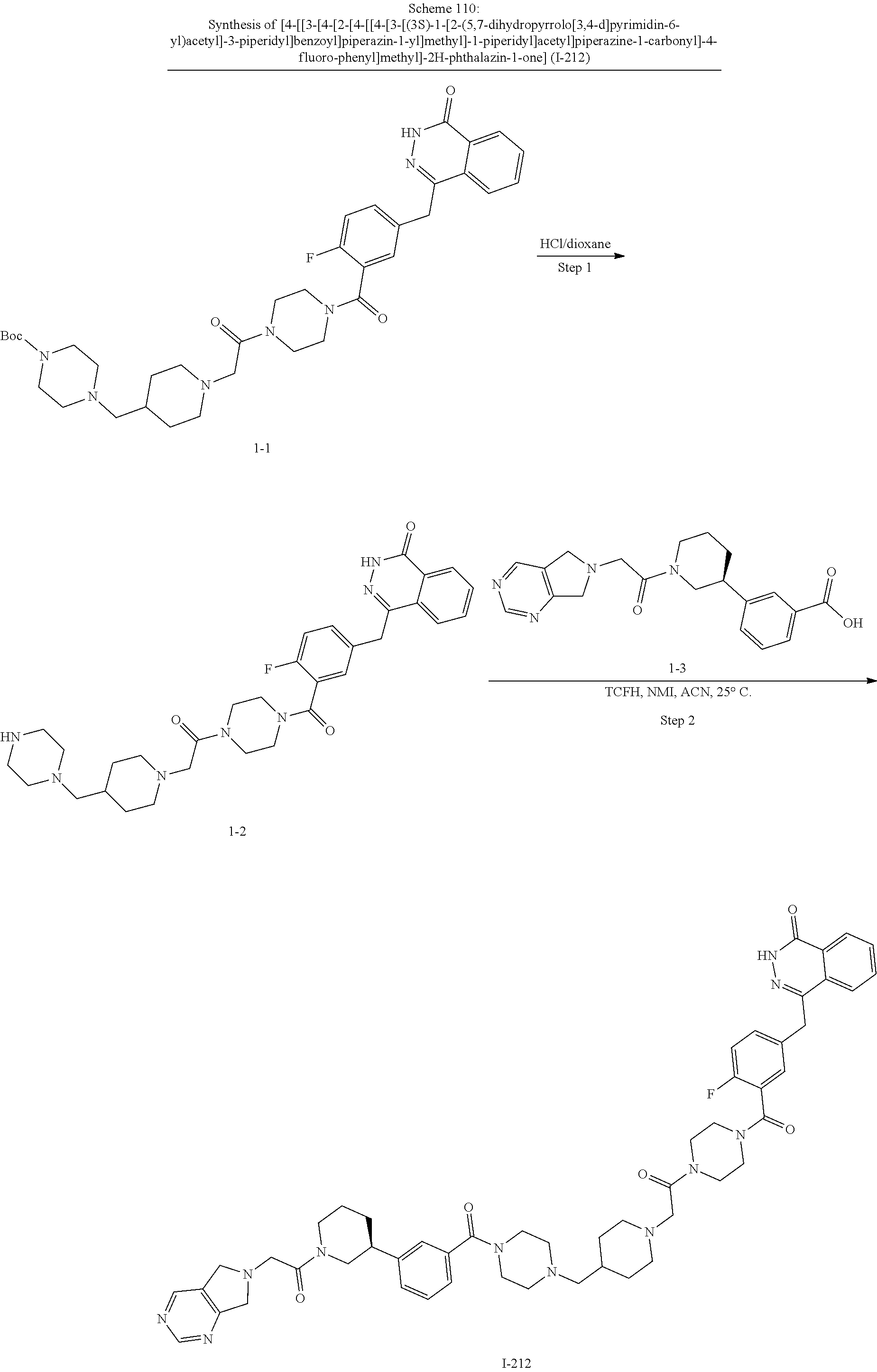

### Step 1: Synthesis of Intermediate 1-2.

To a mixture of intermediate 1-1 (240 mg, 347.92 μmol, 1 eq) in DCM (1 mL) was added HCl/dioxane (2 M, 2.00 mL, 11.50 eq) at 25° C. and the mixture was stirred at 25° C. for 1 hr. The reaction mixture was concentrated to give the crude product. The crude product was used for next the step directly. Intermediate 1-2 (217 mg, crude, HCl salt) was obtained as a white solid. LCMS (Method D): Retention time: 0.176 min, (M+1)=590.2.

### Step 2: Synthesis of I-212

To a mixture of intermediate 1-2 (50 mg, 79.85 μmol, 1 eq, HCl salt) in ACN (0.5 mL) was added intermediate 1-3 (35.11 mg, 95.82 μmol, 1.2 eq), TCFH (26.89 mg, 95.82 μmol, 1.2 eq) and NMI (22.95 mg, 279.48 μmol, 22.28 μL, 3.5 eq) and the mixture was stirred at 25° C. for 1 hr. The reaction mixture was concentrated to give a residue. The residue was purified by prep-HPLC (column: Waters Xbridge 150*25 mm*5 um; mobile phase: [water(NH₃H₂O)-ACN]; gradient: 18%-48% B over 10 min) and lyophilized to give I-212 (17.08 mg, 18.14 μmol, 22.72% yield, 99.63% purity) as a white solid. LCMS (Method F): Retention time: 0.531 min, (M+1)=938.7. SFC: Retention time: 1.208 min. ¹H NMR (400 MHz, METHANOL-d4) δ=8.98 (s, 1H), 8.64 (s, 1H), 8.37 (d, J=7.6 Hz, 1H), 7.99-7.79 (m, 3H), 7.52-7.45 (m, 1H), 7.45-7.22 (m, 5H), 7.16 (t, J=9.2 Hz, 1H), 4.58 (s, 3H), 4.39 (s, 2H), 4.23-4.11 (m, 4H), 4.08-3.99 (m, 1H), 3.93-3.81 (m, 1H), 3.81-3.71 (m, 5H), 3.69-3.64 (m, 1H), 3.59-3.49 (m, 2H), 3.47-3.37 (m, 2H), 3.13 (s, 4H), 2.96-2.85 (m, 2H), 2.84-2.67 (m, 2H), 2.56-2.44 (m, 2H), 2.43-2.32 (m, 2H), 2.28-2.18 (m, 2H), 2.14-1.98 (m, 3H), 1.91-1.72 (m, 4H), 1.69-1.51 (m, 2H), 1.32-1.16 (m, 2H).

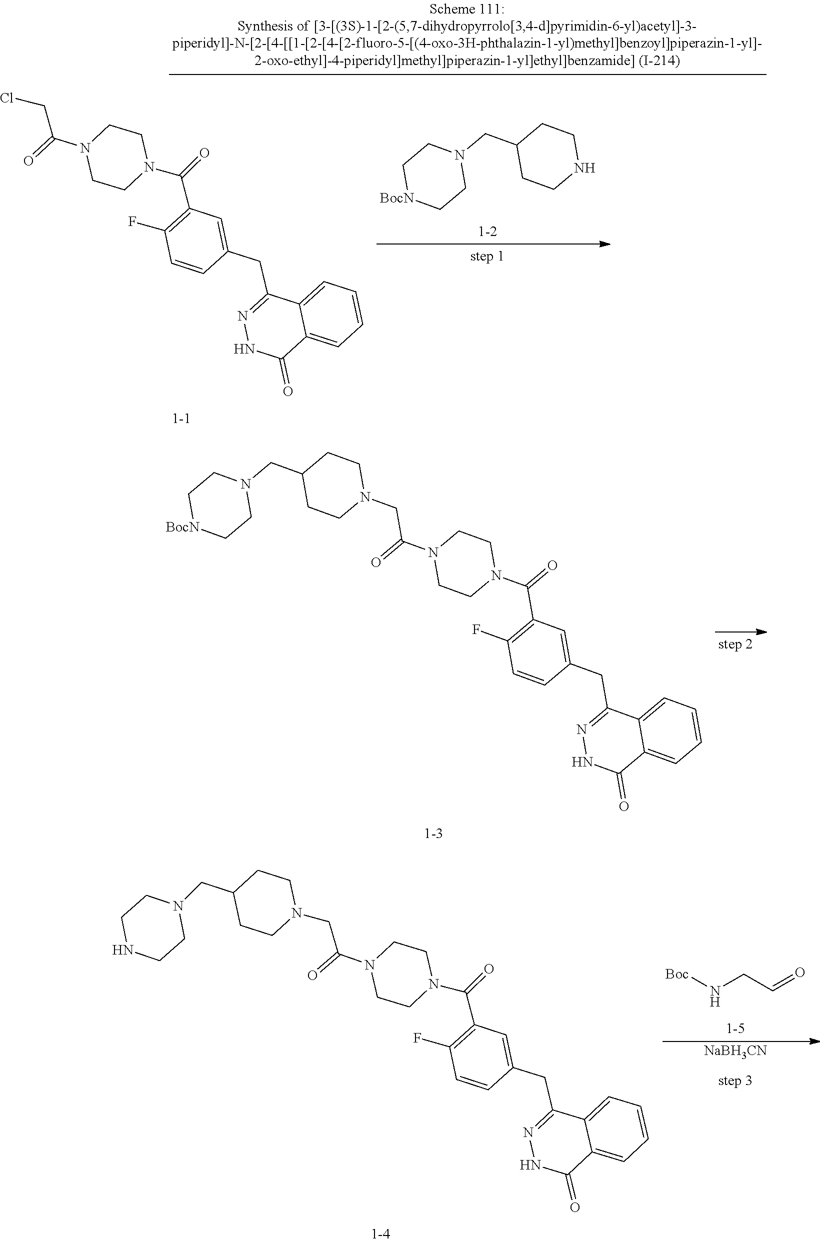 [SMILES: O=C(CCl)N1CCN(C(=O)c2cc(Cc3n[nH]c(=O)c4ccccc34)ccc2F)CC1 ; CC(C)(C)OC(=O)N1CCN(CC2CCNCC2)CC1 ; CC(C)(C)OC(=O)N1CCN(CC2CCN(CC(=O)N3CCN(C(=O)c4cc(Cc5n[nH]c(=O)c6ccccc56)ccc4F)CC3)CC2)CC1 ; O=C(CN1CCC(CN2CCNCC2)CC1)N1CCN(C(=O)c2cc(Cc3n[nH]c(=O)c4ccccc34)ccc2F)CC1 ; CC(C)(C)OC(=O)NCC=O ; "NaBH3CN"]

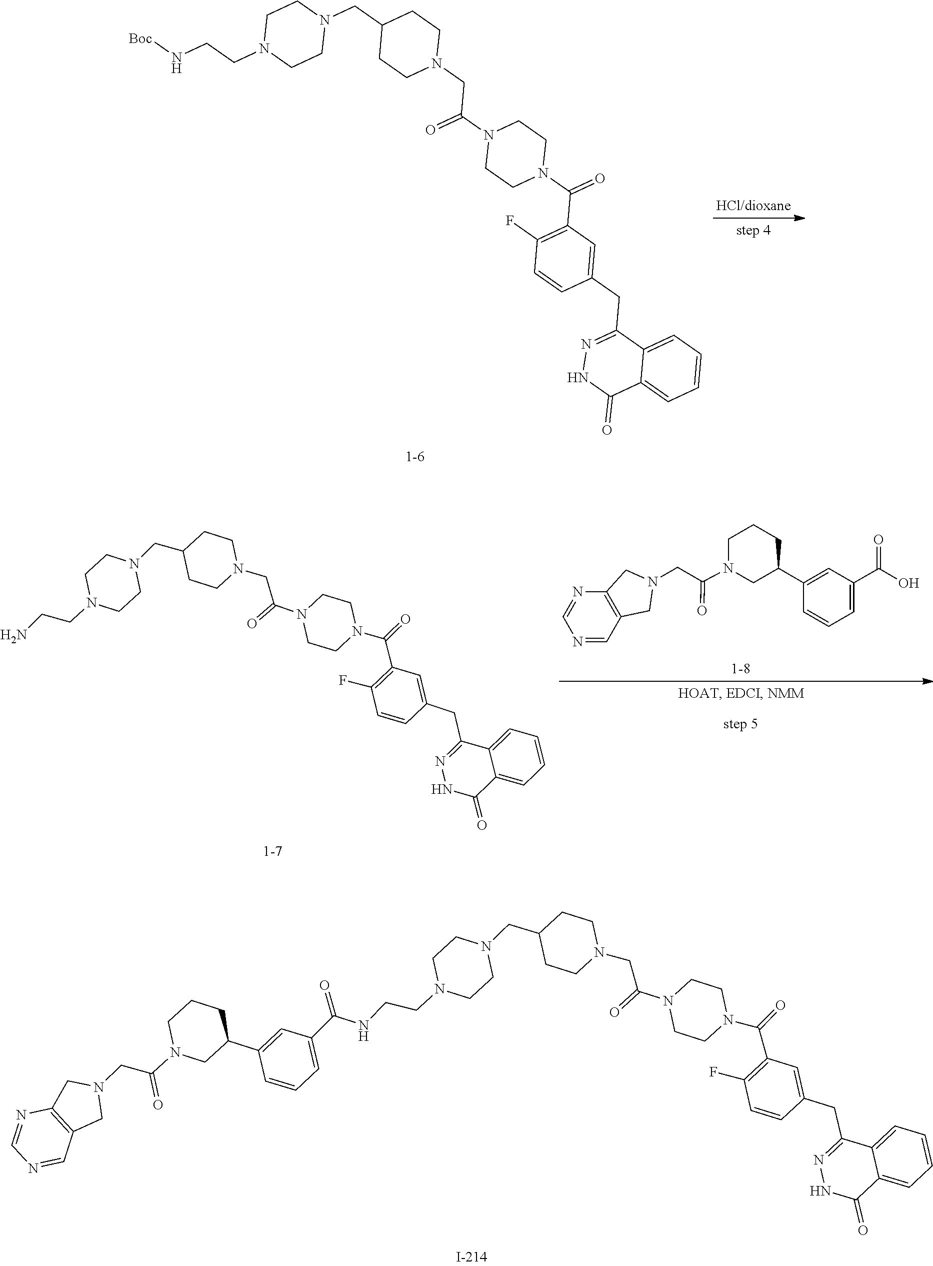 [SMILES: CC(C)(C)OC(=O)NCCN1CCN(CC2CCN(CC(=O)N3CCN(C(=O)c4cc(Cc5n[nH]c(=O)c6ccccc56)ccc4F)CC3)CC2)CC1 ; NCCN1CCN(CC2CCN(CC(=O)N3CCN(C(=O)c4cc(Cc5n[nH]c(=O)c6ccccc56)ccc4F)CC3)CC2)CC1 ; O=C(O)c1cccc([C@@H]2CCCN(C(=O)CN3Cc4cncnc4C3)C2)c1 ; O=C(NCCN1CCN(CC2CCN(CC(=O)N3CCN(C(=O)c4cc(Cc5n[nH]c(=O)c6ccccc56)ccc4F)CC3)CC2)CC1)c1cccc([C@@H]2CCCN(C(=O)CN3Cc4cncnc4C3)C2)c1]

### Step 1: Synthesis of Intermediate 1-3

To a solution of intermediate 1-2 (317.22 mg, 1.12 mmol, 1.2 eq) and intermediate 1-1 (413.08 mg, 932.74 μmol, 1 eq) in ACN (4 mL) was added DIEA (482.20 mg, 3.73 mmol, 649.87 μL, 4 eq). The mixture was stirred at 40° C. for 2 hrs. The reaction mixture was washed with H₂O (10 mL) at 25° C. and the mixture was extracted with DCM (10 ml*3), and the combined organic phase was dried with anhydrous sodium sulfate, filtered and the filtrate was concentrated to give crude product. The crude product was purified by column chromatography (SiO₂, DCM/MeOH=1/0 to 0/1) and the eluent was concentrated under reduced pressure to give intermediate 1-3 (500 mg, 717.58 μmol, 76.93% yield, 99% purity) as a brown solid. LCMS (Method D): Retention time: 0.281 min, (M+H)=690.2.

### Step 2: Synthesis of Intermediate 1-4

A mixture of intermediate 1-3 (450 mg, 652.35 μmol, 1 eq) in HCl/dioxane (2 M, 4.5 mL, 13.80 eq) was stirred at 25° C. for 1 hr. The mixture was concentrated under reduced pressure to give intermediate 1-4 (450 mg, crude, HCl salt) as a white solid. LCMS (Method D): Retention time: 0.240 min, (M+Na)=590.3.

### Step 3: Synthesis of Intermediate 1-6

To a solution of intermediate 1-4 (350 mg, 558.96 μmol, 1 eq, HCl salt) in MeOH (3.5 mL) was added TEA (226.24 mg, 2.24 mmol, 311.20 μL, 4 eq). The mixture was stirred at 25° C. for 15 mins. Then AcOH (167.83 mg, 2.79 mmol, 159.99 μL, 5 eq) and intermediate 1-5 (88.98 mg, 558.96 μmol, 1 eq) were added into the mixture, and the mixture was stirred at 25° C. for 0.5 hr. The NaBH₃CN (140.50 mg, 2.24 mmol, 4 eq) was added into the mixture, and the mixture was stirred at 25° C. for 1 hr. To the reaction mixture was added H₂O (2 mL) at 25° C. The crude product was purified by prep-HPLC (neutral condition) and the eluent was lyophilized to give intermediate 1-6 (200 mg, 264.71 μmol, 47.36% yield, 97% purity) as a white solid. LCMS (Method D): Retention time: 0.291 min, (M−55)=733.4.

### Step 4: Synthesis of Intermediate 1-7

A solution of intermediate 1-6 (200 mg, 272.89 μmol, 1 eq) in HCl/dioxane (2 M, 2 mL, 14.66 eq) was stirred at 25° C. for 1 hr. The mixture was concentrated under reduced pressure to give intermediate 1-7 (200 mg, crude, HCl salt) as a white solid. LCMS (Method D): Retention time: 0.233 min, (M+H)=633.3.

### Step 5: Synthesis of I-214

To a solution of intermediate 1-8 (65.70 mg, 179.31 μmol, 1.5 eq) and intermediate 1-7 (80 mg, 119.54 μmol, 1 eq, HCl) in DMF (0.8 mL) was added TCFH (50.31 mg, 179.31 μmol, 1.5 eq) and NMI (34.35 mg, 418.39 μmol, 33.35 μL, 3.5 eq). The mixture was stirred at 25° C. for 0.5 hr. The mixture was purified by prep-HPLC (column: Waters Xbridge 150*25 mm*5 um; mobile phase: [water(NH₄HCO₃)-ACN]; gradient: 15%-45% B over 14 min) and the eluent was lyophilized to give I-214 (25.09 mg, 24.96 mol, 20.88% yield, 97.619% purity). LCMS (Method D): Retention time: 0.533 min, (M+H)=981.7. SFC: Retention time: 1.180 min. ¹H NMR (400 MHz, METHANOL-d4) δ=8.98 (s, 1H), 8.63 (s, 1H), 8.37 (d, J=7.6 Hz, 1H), 7.99-7.91 (m, 1H), 7.90-7.80 (m, 2H), 7.78-7.65 (m, 2H), 7.51-7.35 (m, 4H), 7.16 (t, J=9.2 Hz, 1H), 4.63-4.57 (m, 1H), 4.38 (s, 2H), 4.23-4.11 (m, 4H), 4.05 (d, J=12.8 Hz, 1H), 3.90-3.82 (m, 1H), 3.82-3.70 (m, 4H), 3.67 (s, 1H), 3.59-3.49 (m, 4H), 3.36-3.32 (m, 1H), 3.28-3.12 (m, 4H), 2.95-2.76 (m, 4H), 2.74-2.66 (m, 1H), 2.61 (d, J=4.4 Hz, 5H), 2.54-2.40 (m, 4H), 2.23-2.20 (m, 2H), 2.10-2.00 (m, 3H), 1.93-1.82 (m, 2H), 1.79-1.63 (m, 3H), 1.59-1.51 (m, 1H), 1.31-1.17 (m, 2H).

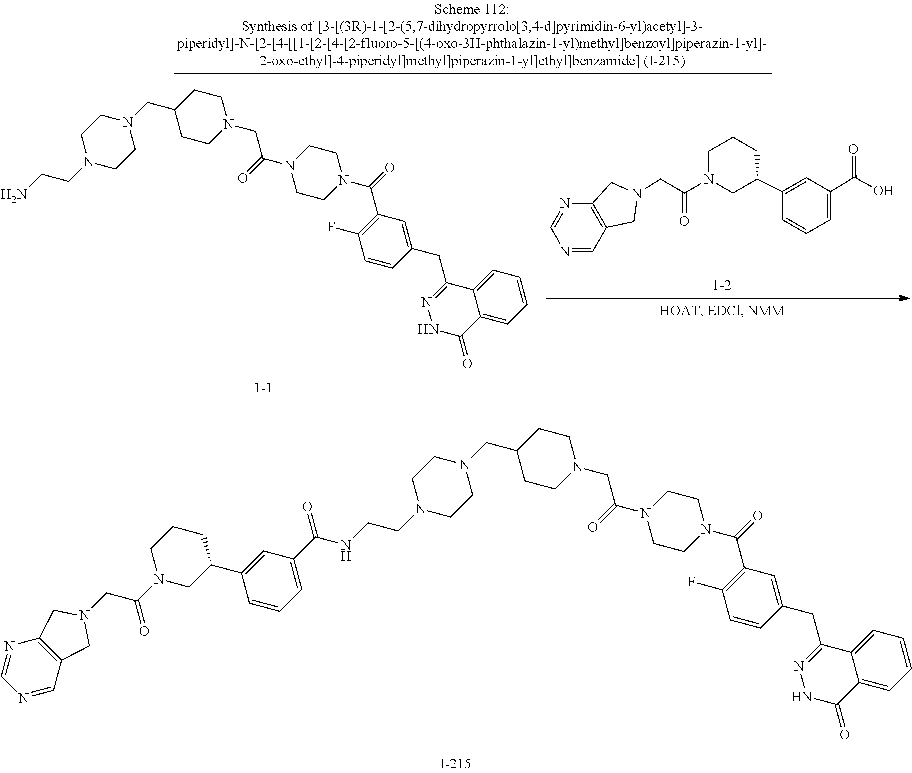 [SMILES: NCCN1CCN(CC2CCN(CC(=O)N3CCN(C(=O)c4cc(Cc5n[nH]c(=O)c6ccccc56)ccc4F)CC3)CC2)CC1 ; O=C(O)c1cccc([C@H]2CCCN(C(=O)CN3Cc4cncnc4C3)C2)c1 ; O=C(NCCN1CCN(CC2CCN(CC(=O)N3CCN(C(=O)c4cc(Cc5n[nH]c(=O)c6ccccc56)ccc4F)CC3)CC2)CC1)c1cccc([C@H]2CCCN(C(=O)CN3Cc4cncnc4C3)C2)c1]

To a solution of intermediate 1-1 (100 mg, 149.43 μmol, 1 eq, HCl salt) and intermediate 1-2 (82.13 mg, 224.14 μmol, 1.5 eq) in DMF (1 mL) was added TCFH (62.89 mg, 224.14 μmol, 1.5 eq) and NMI (42.94 mg, 522.99 μmol, 41.69 μL, 3.5 eq). The mixture was stirred at 25° C. for 0.5 hr. The mixture was purified by prep-HPLC (column: Waters Xbridge 150*25 mm*5 um; mobile phase: [water(NH₄HCO₃)-ACN]; gradient: 15%-45% B over 14 min) and the eluent was lyophilized to give I-215 (22.96 mg, 23.07 mol, 15.44% yield, 98.602% purity). LCMS (Method D): Retention time: 0.529 min, (M+H)=981.7. SFC: Retention time: 2.012 min. ¹H NMR (400 MHz, METHANOL-d4) δ=8.98 (s, 1H), 8.63 (s, 1H), 8.37 (d, J=8.0 Hz, 1H), 7.99-7.91 (m, 1H), 7.91-7.65 (m, 4H), 7.53-7.35 (m, 4H), 7.16 (t, J=8.8 Hz, 1H), 4.62-4.56 (m, 1H), 4.38 (s, 2H), 4.23-4.11 (m, 4H), 4.05 (d, J=13.2 Hz, 1H), 3.89-3.82 (m, 1H), 3.81-3.69 (m, 4H), 3.67 (s, 1H), 3.59-3.49 (m, 4H), 3.36-3.32 (m, 1H), 3.28-3.11 (m, 4H), 2.94-2.75 (m, 4H), 2.74-2.65 (m, 1H), 2.61 (d, J=4.0 Hz, 5H), 2.55-2.41 (m, 4H), 2.22-2.20 (m, 2H), 2.11-2.01 (m, 3H), 1.93-1.82 (m, 2H), 1.79-1.62 (m, 3H), 1.60-1.51 (m, 1H), 1.30-1.16 (m, 2H).

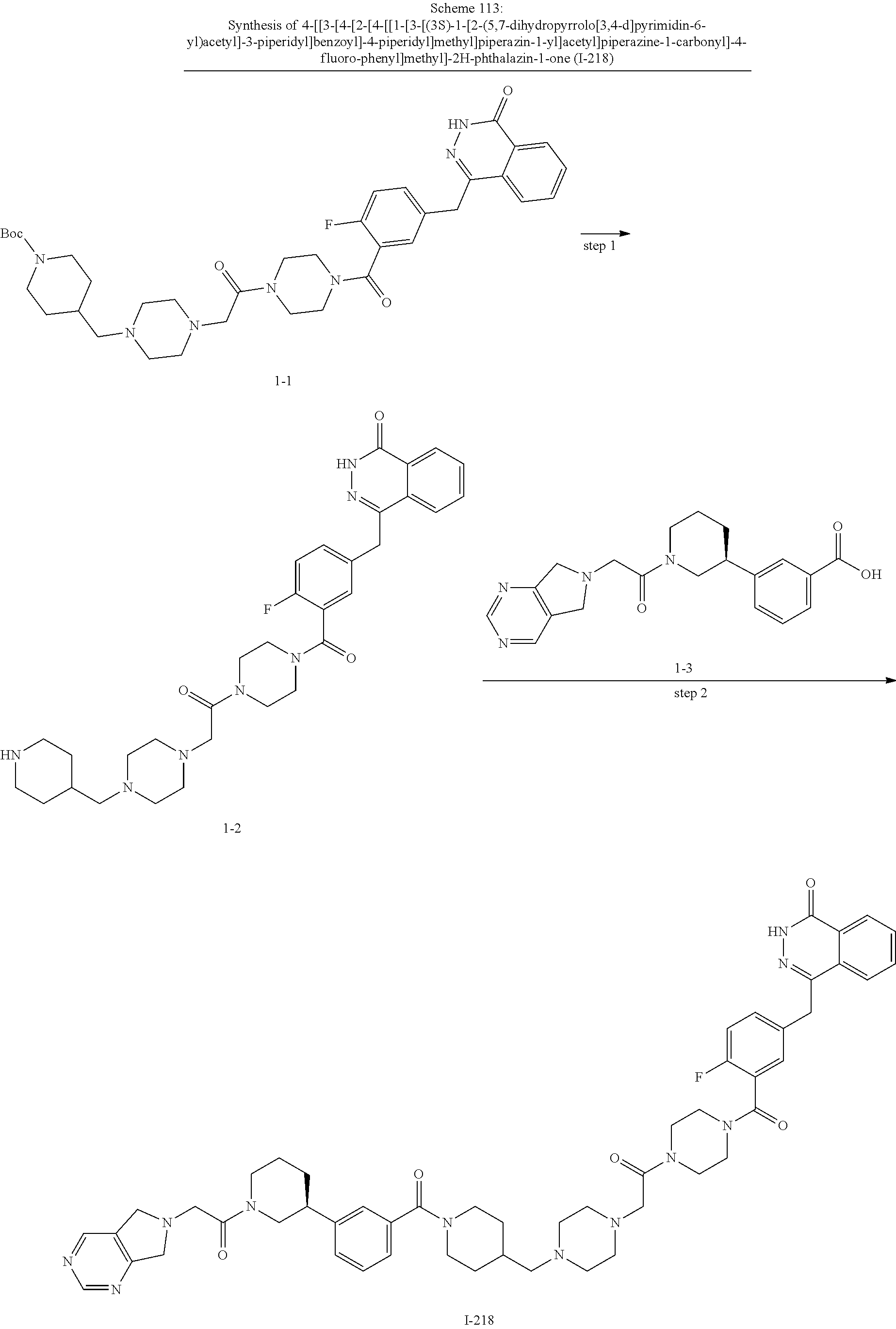 [SMILES: CC(C)(C)OC(=O)N1CCC(CN2CCN(CC(=O)N3CCN(C(=O)c4cc(Cc5n[nH]c(=O)c6ccccc56)ccc4F)CC3)CC2)CC1 ; O=C(CN1CCN(CC2CCNCC2)CC1)N1CCN(C(=O)c2cc(Cc3n[nH]c(=O)c4ccccc34)ccc2F)CC1 ; O=C(O)c1cccc([C@@H]2CCCN(C(=O)CN3Cc4cncnc4C3)C2)c1 ; O=C(CN1CCN(CC2CCN(C(=O)c3cccc([C@@H]4CCCN(C(=O)CN5Cc6cncnc6C5)C4)c3)CC2)CC1)N1CCN(C(=O)c2cc(Cc3n[nH]c(=O)c4ccccc34)ccc2F)CC1]

### Step 1: Synthesis of Intermediate 1-2

To a solution of Intermediate 1-1 (600 mg, 869.80 μmol, 1 eq) in DCM (4 mL) was added TFA (3.07 g, 26.92 mmol, 2 mL). The mixture was stirred at 25° C. for 1 hr. The reaction mixture was concentrated under reduced pressure to give a residue. The residue was used in the next step without further purification. Intermediate 1-2 (710 mg, TFA salt) was obtained as a yellow oil. LCMS (Method D): Rt=0.247 min, [M+H]⁺=590.2.

### Step 2: Synthesis of I-218

To a solution of Intermediate 1-2 (100 mg, 159.70 μmol, 1 eq, HCl salt) and Intermediate 1-3 (58.52 mg, 159.70 μmol, 1 eq) in DMF (1 mL) was added TCFH (67.21 mg, 239.55 μmol, 1.5 eq) and NMI (45.89 mg, 558.96 μmol, 44.56 μL, 3.5 eq). The mixture was stirred at 25° C. for 1 hr. The residue was purified by prep-HPLC (column: Waters Xbridge 150*25 mm*5 um; mobile phase: [water(NH₃H₂O)-ACN]; gradient: 17%-47% B over 10 min). The eluent was lyophilization to give I-218 (20 mg, 19.61 μmol, 12.28% yield, 91.990% purity) as a white solid. LCMS (Method D): Rt=0.299 min, M+H=938.4. SFC: Rt=1.128 min. ¹H NMR (400 MHz, METHANOL-d₄) δ=9.00 (s, 1H), 8.65 (s, 1H), 8.39 (d, J=7.6 Hz, 1H), 8.01-7.94 (m, 1H), 7.93-7.80 (m, 2H), 7.53-7.48 (m, 1H), 7.45-7.36 (m, 3H), 7.35-7.24 (m, 2H), 7.18 (t, J=9.2 Hz, 1H), 4.64-4.55 (m, 3H), 4.40 (s, 2H), 4.28-4.11 (m, 5H), 4.10-4.01 (m, 1H), 3.94-3.65 (m, 8H), 3.61-3.50 (m, 2H), 3.24-3.08 (m, 3H), 2.93-2.70 (m, 4H), 2.62-2.44 (m, 7H), 2.31-2.22 (m, 2H), 2.09-2.03 (m, 1H), 1.94-1.84 (m, 4H), 1.77-1.60 (m, 2H), 1.28-1.10 (m, 2H). ¹⁹F NMR (376 MHz, METHANOL-d₄) δ=−120.802.

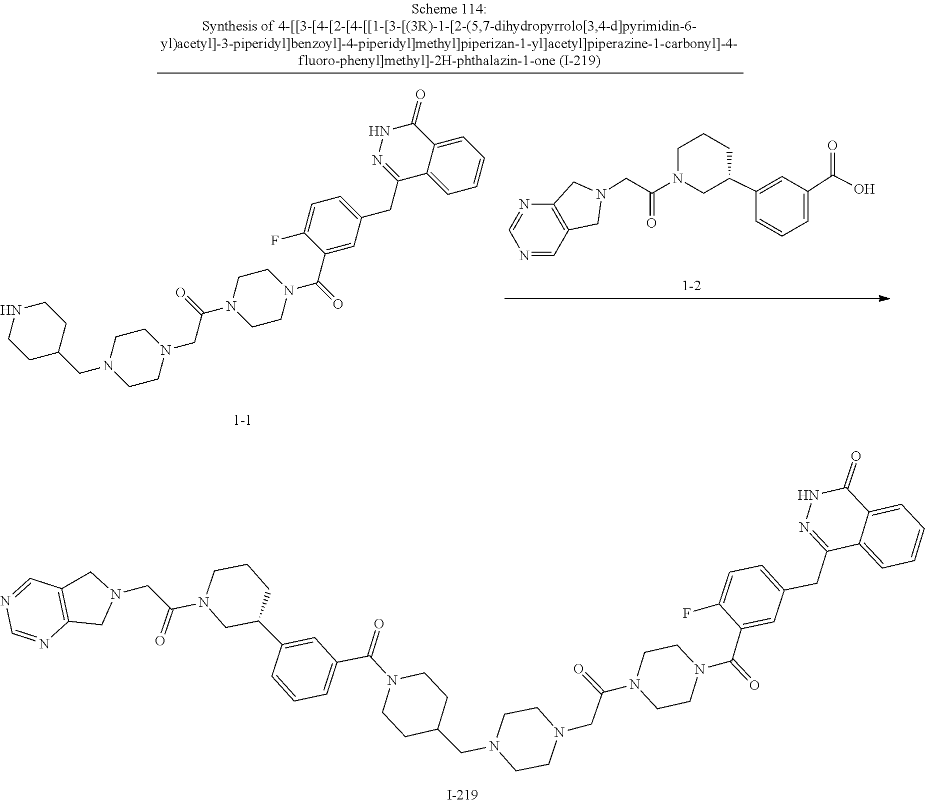 [SMILES: O=C(CN1CCN(CC2CCNCC2)CC1)N1CCN(C(=O)c2cc(Cc3n[nH]c(=O)c4ccccc34)ccc2F)CC1 ; O=C(O)c1cccc([C@H]2CCCN(C(=O)CN3Cc4cncnc4C3)C2)c1 ; O=C(CN1CCN(CC2CCN(C(=O)c3cccc([C@H]4CCCN(C(=O)CN5Cc6cncnc6C5)C4)c3)CC2)CC1)N1CCN(C(=O)c2cc(Cc3n[nH]c(=O)c4ccccc34)ccc2F)CC1]

To a solution of Intermediate 1-1 (80 mg, 127.76 μmol, 1 eq, HCl salt) and Intermediate 1-2 (46.81 mg, 127.76 μmol, 1 eq) in ACN (1 mL) was added TCFH (53.77 mg, 191.64 μmol, 1.5 eq) and NMI (36.71 mg, 447.17 μmol, 35.64 μL, 3.5 eq). The mixture was stirred at 25° C. for 1 hr. The reaction mixture was concentrated under reduced pressure to give a residue. The residue was purified by prep-HPLC (column: Waters Xbridge 150*25 mm*5 um; mobile phase: [water(NH₃H₂O)-ACN]; gradient: 16%-46% B over 14 min). The eluent was lyophilized to give I-219 (15 mg, 15.99 μmol, 12.52% yield, 100% purity) as a white solid. LCMS (Method D): Rt=0.516 min, M+H=938.6. SFC: Rt=1.487 min. ¹H NMR (400 MHz, METHANOL-d4) δ=8.98 (s, 1H), 8.64 (s, 1H), 8.37 (d, J=7.6 Hz, 1H), 7.95 (d, J=5.6 Hz, 1H), 7.91-7.79 (m, 2H), 7.51-7.48 (m, 1H), 7.44-7.30 (m, 4H), 7.24 (d, J=8.0 Hz, 1H), 7.16 (t, J=8.8 Hz, 1H), 4.57 (s, 3H), 4.39 (s, 2H), 4.24-4.09 (m, 5H), 4.07-4.00 (m, 1H), 3.91-3.64 (m, 8H), 3.58-3.48 (m, 2H), 3.23-3.10 (m, 3H), 2.91-2.67 (m, 4H), 2.59-2.40 (m, 8H), 2.28-2.19 (m, 2H), 2.06-2.03 (m, 1H), 1.91-1.81 (m, 4H), 1.75-1.59 (m, 2H), 1.33-1.26 (m, 1H). ¹⁹F NMR (376 MHz, METHANOL-d4), δ=−120.809.

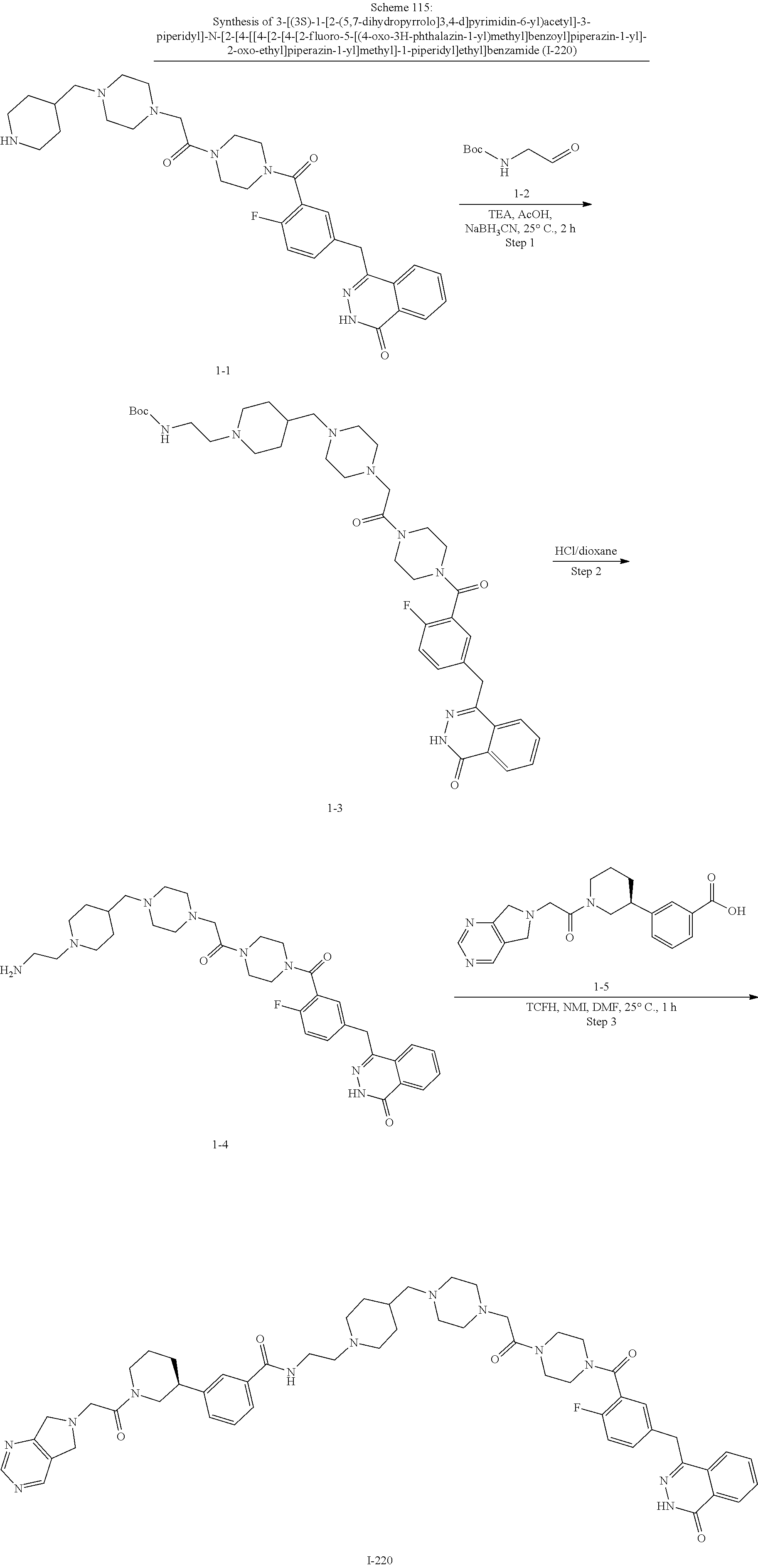 [SMILES: O=C(CN1CCN(CC2CCNCC2)CC1)N1CCN(C(=O)c2cc(Cc3n[nH]c(=O)c4ccccc34)ccc2F)CC1 ; CC(C)(C)OC(=O)NCC=O ; CC(C)(C)OC(=O)NCCN1CCC(CN2CCN(CC(=O)N3CCN(C(=O)c4cc(Cc5n[nH]c(=O)c6ccccc56)ccc4F)CC3)CC2)CC1 ; NCCN1CCC(CN2CCN(CC(=O)N3CCN(C(=O)c4cc(Cc5n[nH]c(=O)c6ccccc56)ccc4F)CC3)CC2)CC1 ; O=C(O)c1cccc([C@@H]2CCCN(C(=O)CN3Cc4cncnc4C3)C2)c1 ; O=C(NCCN1CCC(CN2CCN(CC(=O)N3CCN(C(=O)c4cc(Cc5n[nH]c(=O)c6ccccc56)ccc4F)CC3)CC2)CC1)c1cccc([C@@H]2CCCN(C(=O)CN3Cc4cncnc4C3)C2)c1]

### Step 1: Synthesis of Intermediate 1-3

To a solution of intermediate 1-1 (330 mg, 527.02 μmol, 1 eq, HCl salt) in MeOH (3 mL) was added TEA (213.31 mg, 2.11 mmol, 293.42 μL, 4 eq) dropwise at 25° C. for 10 min. Then intermediate 1-2 (125.84 mg, 790.53 gmol, 1.5 eq) and HOAc (126.59 mg, 2.11 mmol, 120.68 μL, 4 eq) was added at 25° C. for 20 min. Then NaBH₃CN (198.71 mg, 3.16 mmol, 6 eq) was added, and the resulting mixture was stirred at 25° C. for 1 hr. The mixture was diluted with water (5 mL) and extracted with DCM (5 mL*3). The combined organic layer was dried over Na₂SO₄, filtered and concentrated in vacuo to give a residue. The residue was purified by reverse phase HPLC (neutral condition), then the residue was concentrated in vacuo and lyophilized. Intermediate 1-3 (150 mg, 182.16 μmol, 34.56% yield, 89% purity) was obtained as a white solid. LCMS (Method H): Retention time=0.545 min, (M+H)=733.1.

### Step 2: Synthesis of Intermediate 1-4

To a solution of intermediate 1-3 (120 mg, 163.74 μmol, 1 eq) in DCM (0.3 mL) was added HCl/dioxane (2 M, 800.00 L) and it was stirred at 25° C. for 20 min. The reaction mixture was concentrated in vacuo to give a residue. The residue was used in the next step directly without further purification. Intermediate 1-4 (200 mg, crude) was obtained as a white solid. LCMS (Method D): Retention time=0.222 min, (M+H)=633.6.

### Step 3: Synthesis of I-220

To a solution of intermediate 1-4 (60 mg, 89.66 μmol, 1 eq, HCl salt) and intermediate 1-5 (39.42 mg, 107.59 μmol, 1.2 eq) in DMF (0.5 mL) was added TCFH (37.73 mg, 134.48 μmol, 1.5 eq) and NMI (25.76 mg, 313.79 μmol, 25.01 μL, 3.5 eq). The mixture was stirred at 25° C. for 30 min. The mixture was diluted with water (5 mL) and extracted with DCM (5 mL*3). The combined organic layer was dried over Na₂SO₄, filtered and concentrated in vacuo to give a residue. The residue was purified by prep-HPLC (column: Waters Xbridge BEH C18 150*25 mm*5 um; mobile phase: [water(NH₄HCO₃)-ACN]; gradient: 16%-46% B over 10 min). Then eluent was concentrated in vacuo and lyophilized. I-220 (13.59 mg, 13.43 μmol, 14.98% yield, 96.974% purity) was obtained as a white solid. LMCS (Method D): Retention time=0.269 min, (M+H)=981.4. SFC: Retention time=1.145 min. ¹H NMR (400 MHz, METHANOL-d₄) δ=8.98 (s, 1H), 8.64 (s, 1H), 8.37 (d, J=8.0 Hz, 1H), 8.00-7.93 (m, 1H), 7.91-7.86 (m, 1H), 7.86-7.81 (m, 1H), 7.77-7.66 (m, 2H), 7.52-7.43 (m, 2H), 7.43-7.34 (m, 2H), 7.16 (t, J=8.8 Hz, 1H), 4.90 (s, 2H), 4.61-4.57 (m, 1H), 4.39 (s, 2H), 4.20 (d, J=10.4 Hz, 2H), 4.15 (d, J=12.4 Hz, 2H), 4.08-4.02 (m, 1H), 3.88-3.79 (m, 2H), 3.77-3.65 (m, 4H), 3.56 (d, J=4.5 Hz, 3H), 3.52-3.48 (m, 1H), 3.27 (s, 1H), 3.22-3.14 (m, 2H), 3.07-3.01 (m, 2H), 2.90-2.68 (m, 3H), 2.65-2.60 (m, 2H), 2.58-2.41 (m, 7H), 2.24-2.18 (m, 2H), 2.15-2.03 (m, 3H), 1.91-1.77 (m, 4H), 1.70-1.56 (m, 2H), 1.31-1.23 (m, 2H). ¹⁹F NMR (376 MHz, METHANOL-d₄) δ=−120.740

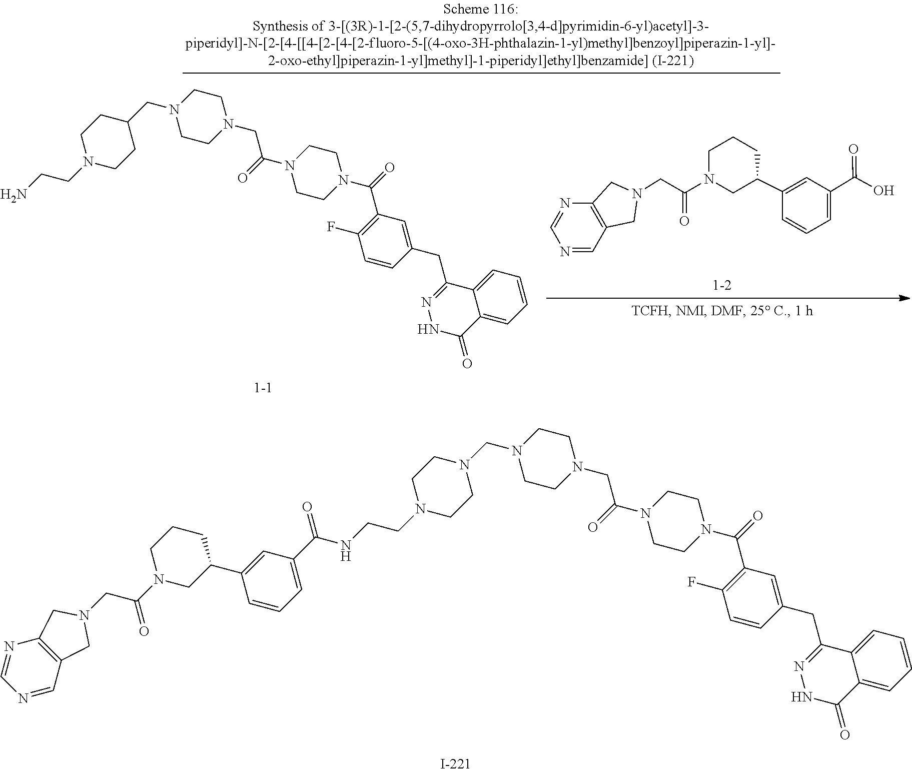 [SMILES: NCCN1CCC(CN2CCN(CC(=O)N3CCN(C(=O)c4cc(Cc5n[nH]c(=O)c6ccccc56)ccc4F)CC3)CC2)CC1 ; O=C(O)c1cccc([C@H]2CCCN(C(=O)CN3Cc4cncnc4C3)C2)c1 ; O=C(NCCN1CCN(CN2CCN(CC(=O)N3CCN(C(=O)c4cc(Cc5n[nH]c(=O)c6ccccc56)ccc4F)CC3)CC2)CC1)c1cccc([C@H]2CCCN(C(=O)CN3Cc4cncnc4C3)C2)c1]

To a solution of intermediate 1-1 (60 mg, 89.66 μmol, 1 eq, HCl salt) and intermediate 1-2 (39.42 mg, 107.59 μmol, 1.2 eq) in DMF (1 mL) was added TCFH (37.73 mg, 134.48 μmol, 1.5 eq) and NMI (25.76 mg, 313.79 μmol, 25.01 μL, 3.5 eq). The mixture was stirred at 25° C. for 30 min. The mixture was diluted with water (5 mL) and extracted with DCM (5 mL*3). The combined organic layer was dried over Na₂SO₄, filtered and concentrated in vacuo to give a residue. The residue was purified by prep-HPLC (column: Waters Xbridge BEH C18 150*25 mm*5 um; mobile phase: [water (NH₄HCO₃)-ACN]; gradient: 16%-46% B over 10 min). Then the eluent was concentrated in vacuo and lyophilized. I-221 (16.33 mg, 15.31 μmol, 17.08% yield, 92.009% purity) was obtained as a white solid. LCMS (Method D): Retention time=0.269 min, (M+H)=981.4. SFC: Retention time=1.894 min. ¹H NMR (400 MHz, METHANOL-d₄) δ=8.98 (s, 1H), 8.63 (s, 1H), 8.37 (d, J=7.2 Hz, 1H), 7.99-7.92 (m, 1H), 7.91-7.80 (m, 2H), 7.75 (d, J=16.4 Hz, 1H), 7.69 (t, J=7.6 Hz, 1H), 7.53-7.43 (m, 2H), 7.43-7.35 (m, 2H), 7.16 (t, J=8.8 Hz, 1H), 4.68-4.48 (m, 2H), 4.39 (s, 2H), 4.20 (d, J=10.4 Hz, 2H), 4.15 (d, J=12.4 Hz, 2H), 4.05-4.03 (m, 1H), 3.89-3.79 (m, 2H), 3.79-3.69 (m, 3H), 3.67 (s, 1H), 3.56 (s, 3H), 3.51 (s, 1H), 3.27 (s, 2H), 3.22-3.13 (m, 2H), 3.03 (d, J=8.4 Hz, 2H), 2.86-2.72 (m, 2H), 2.69-2.58 (m, 3H), 2.57-2.46 (m, 5H), 2.45-2.32 (m, 2H), 2.21-2.04 (m, 2H), 2.14-2.03 (m, 3H), 1.94-1.84 (m, 2H), 1.82-1.76 (m, 2H), 1.70-1.53 (m, 2H), 1.31-1.24 (m, 2H). ¹⁹F NMR (376 MHz, METHANOL-d₄) δ=−120.807.

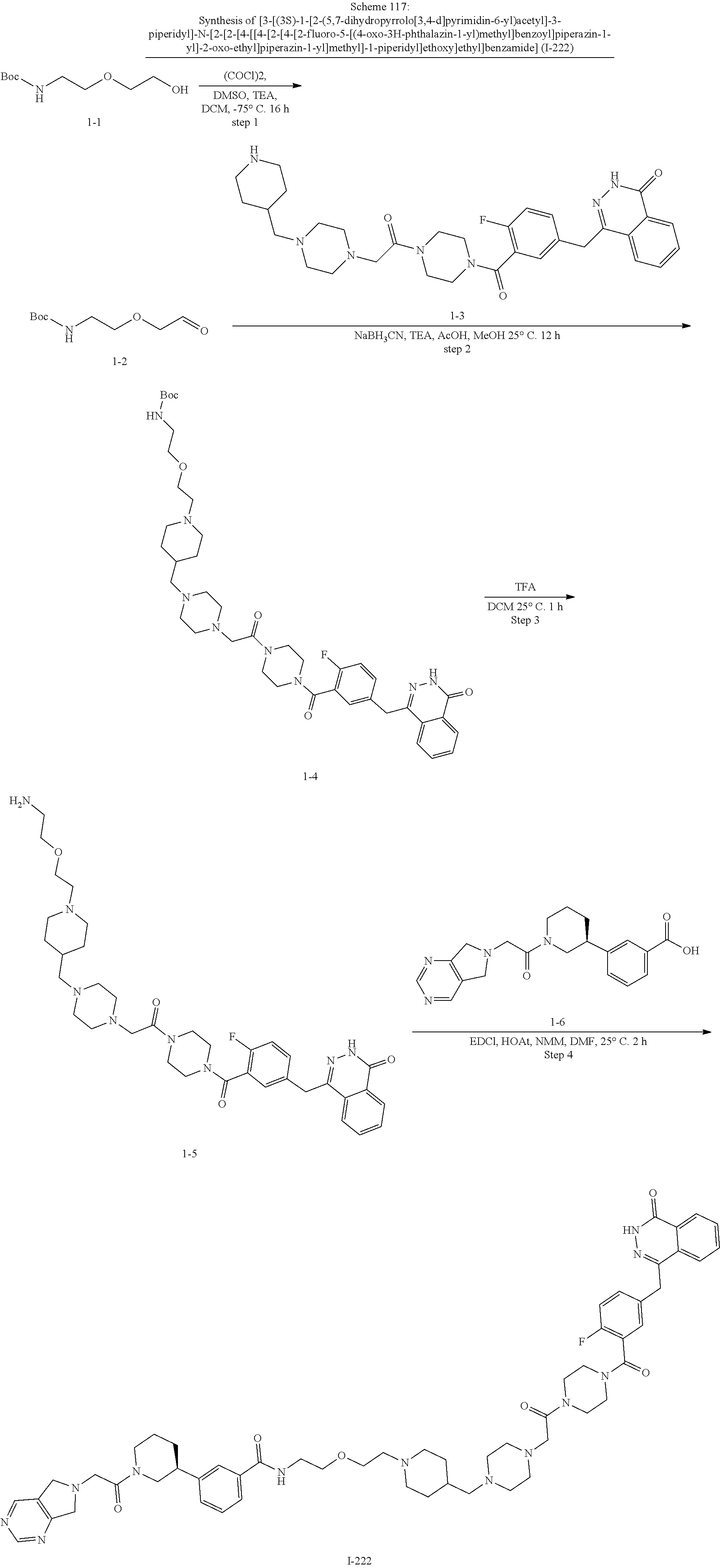

### Step 1: Synthesis of Intermediate 1-2

To a solution of DMSO (1.90 g, 24.36 mmol, 1.90 mL, 5 eq) in DCM (10 mL) was added dropwise a solution of (COCl)₂ (1.42 g, 11.21 mmol, 980.90 μL, 2.3 eq) in DCM (10 mL) at −78° C. under N2 atmosphere. Then a solution of intermediate 1-1 (1 g, 4.87 mmol, 1 eq) in DCM (10 mL) was added, and it was stirred at −78° C. for 0.5 h. Then the TEA (3.94 g, 38.98 mmol, 5.43 mL, 8 eq) was added into the mixture and it was stirred at −78° C. for 3 h. The reaction mixture was poured into water (50 mL) and extracted with EA (20 mL*4). The combined organic layers were dried over Na₂SO₄, filtered and concentrated under reduced pressure to give a residue. The crude product intermediate 1-2 (700 mg, 3.44 mmol, 70.69% yield) was a yellow oil and used in the next step without further purification. ¹H NMR (400 MHz, CHLOROFORM-d) δ=5.33 (br s, 1H), 3.86 (br d, J=12.0 Hz, 2H), 3.57-3.48 (m, 2H), 2.76-2.71 (m, 2H), 1.42 (s, 9H).

### Step 2: Synthesis of Intermediate 1-4

To a solution of intermediate 1-3 (150 mg, 213.15 μmol, 1 eq, TFA salt) in MeOH (1.5 mL) was added TEA (129.41 mg, 1.28 mmol, 178.01 μL, 6 eq) to adjust the pH to 7-8 and it was stirred at 25° C. for 5 min then AcOH (76.80 mg, 1.28 mmol, 73.21 μL, 6 eq) was added to adjust the pH to 6-5. Intermediate 1-2 (64.98 mg, 319.73 μmol, 1.5 eq) was added in and it was stirred at 25° C. for 5 min. NaBH₃CN (53.58 mg, 852.61 μmol, 4 eq) was added. The mixture was stirred at 25° C. for 12 h 50 min. The residue was diluted with H₂O (5 mL) and extracted with EA (5 mL*3), dried over Na₂SO₄, filtered and concentrated under reduced pressure to give a residue. The crude product was purified by reversed-phase HPLC (neutral condition). The eluent was concentrated under reduced pressure to remove ACN, then lyophilized to afford product. Intermediate 1-4 (200 mg, crude) was obtained as yellow oil. LCMS (Method H): Rt=0.537 min, [M+H]⁺=777.2.

### Step 3: Synthesis of Intermediate 1-5

To a solution of intermediate 1-4 (150 mg, 193.07 μmol, 1 eq) in DCM (1 mL) was added TFA (0.5 mL). The mixture was stirred at 25° C. for 1 hr. The reaction mixture was concentrated under reduced pressure to give a residue, then washed by PE (3*1 mL) and concentrated under reduced pressure to give a residue. The crude product intermediate 1-5 (150 mg, 189.67 μmol, 98.24% yield, TFA salt) was a yellow oil and was used in the next step without further purification.

### Step 4: Synthesis of I-222

To a solution of intermediate 1-5 (100 mg, 147.75 μmol, 1 eq) and intermediate 1-6 (54.14 mg, 147.75 μmol, 1 eq) in DMF (1 mL) was added TCFH (62.18 mg, 221.62 μmol, 1.5 eq) and NMI (42.46 mg, 517.12 μmol, 41.22 μL, 3.5 eq). The mixture was stirred at 25° C. for 1 hr. The residue was purified by prep-HPLC (column: Waters Xbridge 150*25 mm*5 um; mobile phase: [water(NH₃H₂O)-ACN]; gradient: 22%-42% B over 14 min). The eluent was lyophilized to give a residue. The residue was purified by prep-HPLC (column: Phenomenex Luna C18 100*30 mm*5 um; mobile phase: [water(FA)-ACN]; gradient: 0%-28% B over 10 min). The eluent was lyophilized to give I-222 (10 mg, 9.75 μmol, 6.60% yield, 100% purity) as an orange solid. LCMS (Method D): Rt=0.278 min, [M+H]⁺=1025.4. SFC: Retention time: 0.649 min H NMR (400 MHz, METHANOL-d₄) δ=8.98 (s, 1H), 8.63 (s, 1H), 8.46 (s, 1H), 8.38-8.36 (m, 1H), 7.94 (d, J=7.6 Hz, 1H), 7.91-7.80 (m, 2H), 7.79-7.67 (m, 2H), 7.53-7.41 (m, 3H), 7.40-7.33 (m, 1H), 7.17 (t, J=8.8 Hz, 1H), 4.63-4.55 (m, 1H), 4.39 (s, 2H), 4.23-4.11 (m, 4H), 4.05 (d, J=13.6 Hz, 1H), 3.92-3.76 (m, 5H), 3.76-3.58 (m, 8H), 3.57-3.48 (m, 4H), 3.35 (d, J=2.4 Hz, 2H), 3.28 (s, 3H), 3.22-3.12 (m, 1H), 2.99-2.89 (m, 2H), 2.86-2.71 (m, 2H), 2.63-2.44 (m, 7H), 2.24 (s, 2H), 2.09-2.02 (m, 1H), 1.95-1.76 (m, 5H), 1.72-1.57 (m, 1H), 1.46-1.27 (m, 3H). ¹⁹F NMR (376 MHz, METHANOL-d₄) δ=−12.785.

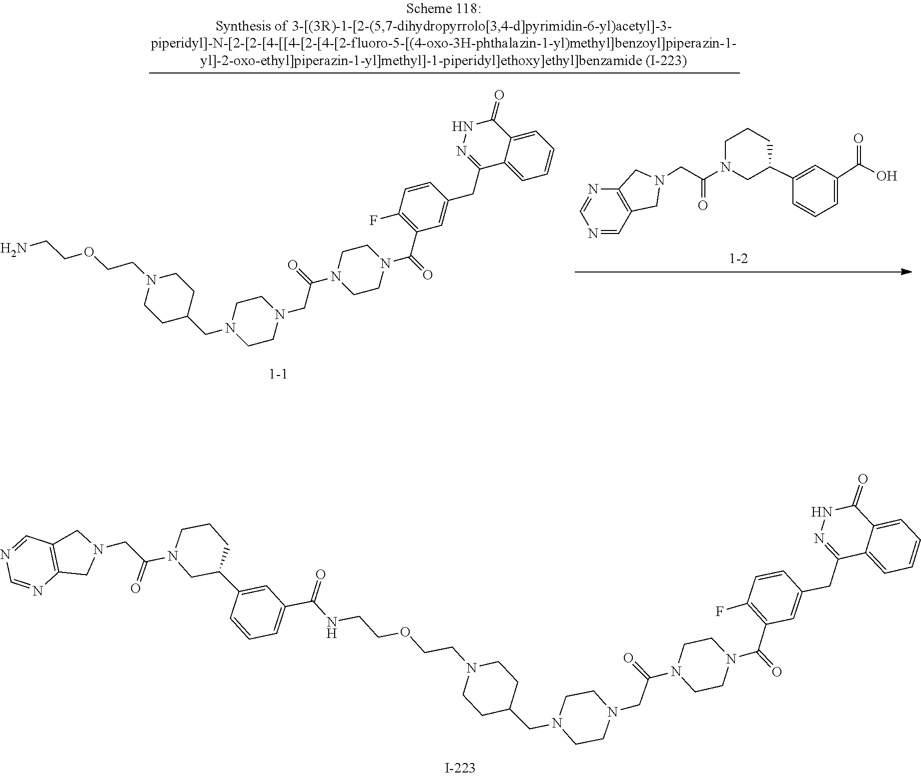 [SMILES: NCCOCCN1CCC(CN2CCN(CC(=O)N3CCN(C(=O)c4cc(Cc5n[nH]c(=O)c6ccccc56)ccc4F)CC3)CC2)CC1 ; O=C(O)c1cccc([C@H]2CCCN(C(=O)CN3Cc4cncnc4C3)C2)c1 ; O=C(NCCOCCN1CCC(CN2CCN(CC(=O)N3CCN(C(=O)c4cc(Cc5n[nH]c(=O)c6ccccc56)ccc4F)CC3)CC2)CC1)c1cccc([C@H]2CCCN(C(=O)CN3Cc4cncnc4C3)C2)c1]

To a solution of Intermediate 1-1 (100 mg, 140.20 μmol, 1 eq, HCl salt) and Intermediate 1-2 (51.37 mg, 140.20 μmol, 1 eq) in DMF (1 mL) was added TCFH (59.00 mg, 210.30 μmol, 1.5 eq) and NMI (40.29 mg, 490.69 μmol, 39.11 μL, 3.5 eq) The mixture was stirred at 25° C. for 1 hr. The residue was purified by prep-HPLC (column: Waters Xbridge BEH C18 150*25 mm*5 um; mobile phase: [water(NH₄HCO₃)-ACN]; gradient: 18%-38% B over 10 min). The eluent was lyophilized to give a residue. The residue was purified by prep-HPLC (column: Phenomenex C18 150 mm*30 mm*5 m; mobile phase: [water(FA)-ACN]; gradient: 0%-28% B over 10 min). The eluent was lyophilized to give I-223 (10 mg, 9.75 mol, 6.96% yield, 100% purity) as white gum. LCMS (Method D): Rt=0.275 min, M+H=1025.5. SFC: Rt=1.003 min. ¹H NMR (400 MHz, METHANOL-d₄) δ=8.97 (s, 1H), 8.63 (s, 1H), 8.42 (s, 1H), 8.36 (d, J=7.6 Hz, 1H), 7.99-7.92 (m, 1H), 7.90-7.80 (m, 2H), 7.80-7.67 (m, 2H), 7.55-7.41 (m, 3H), 7.39-7.34 (m, 1H), 7.17 (t, J=8.8 Hz, 1H), 4.65-4.54 (m, 1H), 4.38 (s, 2H), 4.24-4.10 (m, 4H), 4.05 (d, J=12.8 Hz, 1H), 3.91-3.77 (m, 5H), 3.77-3.59 (m, 8H), 3.52 (s, 4H), 3.42-3.33 (m, 3H), 3.28 (s, 2H), 3.24-3.13 (m, 1H), 2.95 (t, J=12.4 Hz, 2H), 2.84-2.72 (m, 2H), 2.69-2.51 (m, 7H), 2.28 (s, 2H), 2.13-1.98 (m, 2H), 1.96-1.79 (m, 5H), 1.75-1.58 (m, 1H), 1.49-1.34 (m, 2H). ¹⁹F NMR (376 MHz, METHANOL-d₄) δ=−120.667, −120.764.

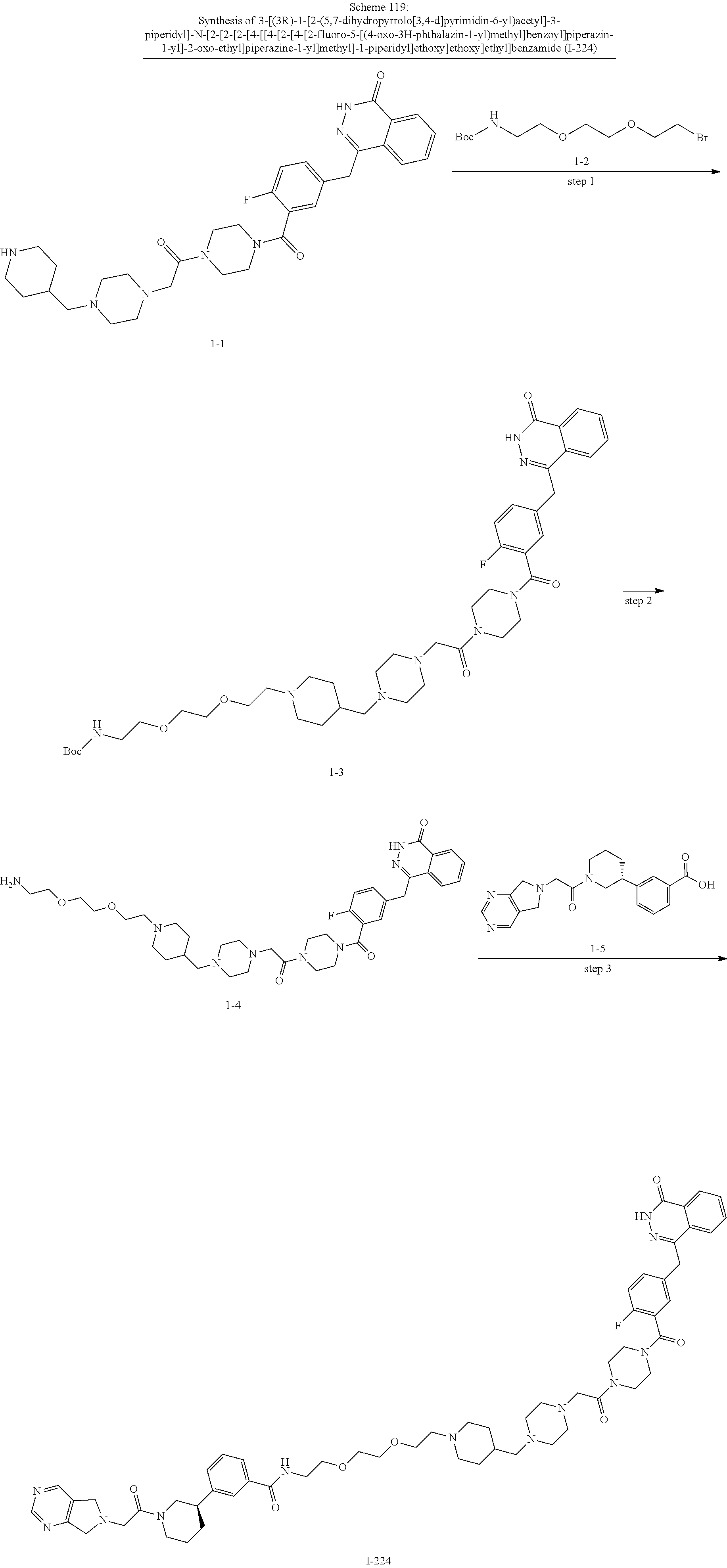 [SMILES: O=C(CN1CCN(CC2CCNCC2)CC1)N1CCN(C(=O)c2cc(Cc3n[nH]c(=O)c4ccccc34)ccc2F)CC1 ; CC(C)(C)OC(=O)NCCOCCOCCBr ; CC(C)(C)OC(=O)NCCOCCOCCN1CCC(CN2CCN(CC(=O)N3CCN(C(=O)c4cc(Cc5n[nH]c(=O)c6ccccc56)ccc4F)CC3)CC2)CC1 ; NCCOCCOCCN1CCC(CN2CCN(CC(=O)N3CCN(C(=O)c4cc(Cc5n[nH]c(=O)c6ccccc56)ccc4F)CC3)CC2)CC1 ; O=C(O)c1cccc([C@H]2CCCN(C(=O)CN3Cc4cncnc4C3)C2)c1 ; O=C(NCCOCCOCCN1CCC(CN2CCN(CC(=O)N3CCN(C(=O)c4cc(Cc5n[nH]c(=O)c6ccccc56)ccc4F)CC3)CC2)CC1)c1cccc([C@H]2CCCN(C(=O)CN3Cc4cncnc4C3)C2)c1]

### Step 1: Synthesis of Intermediate 1-3

To a solution of Intermediate 1-1 (60 mg, 85.26 μmol, 1 eq, TFA salt) in MeOH (1 mL) was added TEA (51.76 mg, 511.56 μmol, 71.20 μL, 6 eq) and it was stirred at 25° C. for 10 min. AcOH (30.72 mg, 511.56 μmol, 29.29 μL, 6 eq) and Intermediate 1-2 (21.08 mg, 85.26 μmol, 1 eq) were added, and it was stirred at 25° C. for 30 min. NaBH₃CN (21.43 mg, 341.04 μmol, 4 eq) was added. The mixture was stirred at 25° C. for 50 min. The reaction mixture was concentrated under reduced pressure to give a residue. The residue was purified by prep-HPLC (Neutral condition), and the eluent was lyophilized to give Intermediate 1-3 (55 mg, 66.99 μmol, 78.57% yield) as a yellow oil. LCMS (Method D): Rt=0.547 min, (M+H)=821.2.

### Step 2: Synthesis of Intermediate 1-4

To a solution of t Intermediate 1-3 (50 mg, 60.90 μmol, 1 eq) in DCM (0.25 mL) was added HCl/dioxane (2 M, 0.25 mL, 8.21 eq). The mixture was stirred at 25° C. for 0.5 hr. The reaction mixture was concentrated under reduced pressure to give a residue. The residue was used into the next step without further purification. Intermediate 1-4 (120 mg, crude, HCl salt) was obtained as white solid. LCMS (Method D): Rt=0.227 min, (M+H)=721.5.

### Step 3: Synthesis of I-224

To a solution of Intermediate 1-4 (100 mg, 132.04 μmol, 1 eq, HCl salt) and Intermediate 1-5 (48.38 mg, 132.04 μmol, 1 eq) in DMF (0.5 mL) was added TCFH (55.57 mg, 198.06 μmol, 1.5 eq) and NMI (37.94 mg, 462.15 μmol, 36.84 μL, 3.5 eq). The mixture was stirred at 25° C. for 1 hr. The residue was purified by prep-HPLC (column: Waters Xbridge BEH C18 150*25 mm*5 um; mobile phase: [water(NH₄HCO₃)-ACN]; gradient: 18%-38% B over 10 min). The eluent was lyophilized to give I-224 (25 mg, 23.38 μmol, 17.71% yield, 100% purity) as a yellow solid. LCMS (Method D): Rt=0.275 min, (M+H)=1069.5. SFC: Rt=1.049 min. ¹H NMR (400 MHz, METHANOL-d₄) δ=8.97 (s, 1H), 8.63 (s, 1H), 8.41-8.33 (m, 1H), 7.96-7.94 (m, 1H), 7.90-7.81 (m, 2H), 7.78-7.67 (m, 2H), 7.51-7.43 (m, 2H), 7.42-7.35 (m, 2H), 7.16 (t, J=8.8 Hz, 1H), 4.59 (d, J=9.6 Hz, 2H), 4.38 (s, 2H), 4.20 (d, J=8.8 Hz, 2H), 4.14 (d, J=11.6 Hz, 2H), 4.05 (d, J=13.2 Hz, 1H), 3.89-3.78 (m, 3H), 3.76-3.70 (m, 3H), 3.66-3.64 (m, 6H), 3.63-3.60 (m, 4H), 3.57 (t, J=5.2 Hz, 3H), 3.51 (s, 1H), 3.22-3.14 (m, 2H), 2.94 (d, J=10.8 Hz, 2H), 2.81-2.68 (m, 2H), 2.59-2.38 (m, 10H), 2.18-2.15 (m, 2H), 2.08-2.00 (m, 3H), 1.92-1.83 (m, 2H), 1.71 (d, J=11.2 Hz, 3H), 1.56-1.46 (m, 1H), 1.26-1.16 (m, 2H). ¹⁹F NMR (376 MHz, METHANOL-d₄) δ=−120.762.

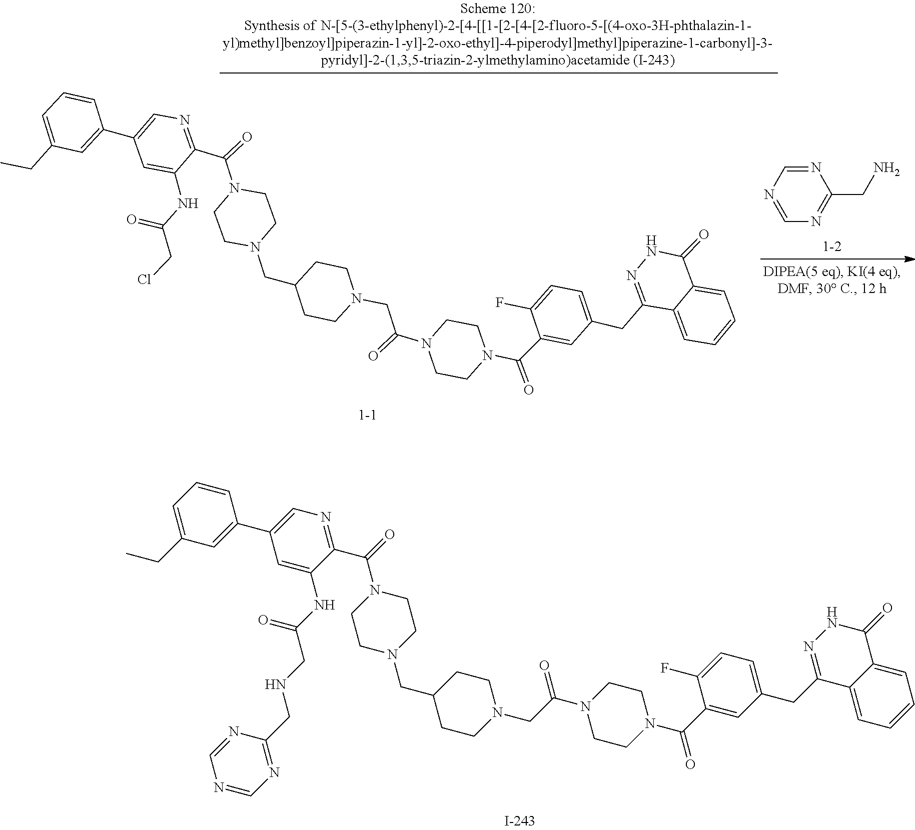 [SMILES: CCc1cccc(-c2cnc(C(=O)N3CCN(CC4CCN(CC(=O)N5CCN(C(=O)c6cc(Cc7n[nH]c(=O)c8ccccc78)ccc6F)CC5)CC4)CC3)c(NC(=O)CCl)c2)c1 ; NCc1ncncn1 ; CCc1cccc(-c2cnc(C(=O)N3CCN(CC4CCN(CC(=O)N5CCN(C(=O)c6cc(Cc7n[nH]c(=O)c8ccccc78)ccc6F)CC5)CC4)CC3)c(NC(=O)CNCc3ncncn3)c2)c1]

A solution of Intermediate 1-2 (37.10 mg, 336.91 μmol, 3 eq) and Intermediate 1-1 (100 mg, 112.30 μmol, 1 eq) in DMF (2 mL) was added DIEA (43.54 mg, 336.91 μmol, 58.68 μL, 3 eq). The reaction mixture was stirred and refluxed at 40° C. for 1 h. The reaction mixture was filtered and concentrated under reduced pressure to give a residue. The crude was purified by prep-HPLC (column: Welch Xtimate C18 150*25 mm*5 um; mobile phase: [water (TFA)-ACN]; gradient: 10%-40% B over 10 min) and the eluent was concentrated to remove ACN and lyophilized to give I-243 (18 mg, 17.04 μmol, 15.17% yield, 91.257% purity) as a brown solid. LCMS (Method D): Retention time: 0.294 min, (M+H)=964.3. ¹H NMR (400 MHz, METHANOL-d₄) δ=8.73-8.66 (m, 1H), 8.39-8.34 (m, 1H), 8.25-8.11 (m, 1H), 7.97-7.93 (m, 1H), 7.88-7.81 (m, 2H), 7.58-7.27 (m, 8H), 7.17 (t, J=8.8 Hz, 1H), 4.39 (s, 2H), 4.34-4.21 (m, 3H), 3.96 (d, J=12.4 Hz, 3H), 3.85-3.66 (m, 6H), 3.58-3.42 (m, 7H), 3.37 (s, 4H), 3.25-3.11 (m, 4H), 2.77-2.71 (m, 2H), 2.37-1.97 (m, 4H), 1.81-1.69 (m, 2H), 1.29 (t, J=7.6 Hz, 3H). ¹⁹F NMR (376 MHz, METHANOL-d₄) δ=−76.971, −120.672.

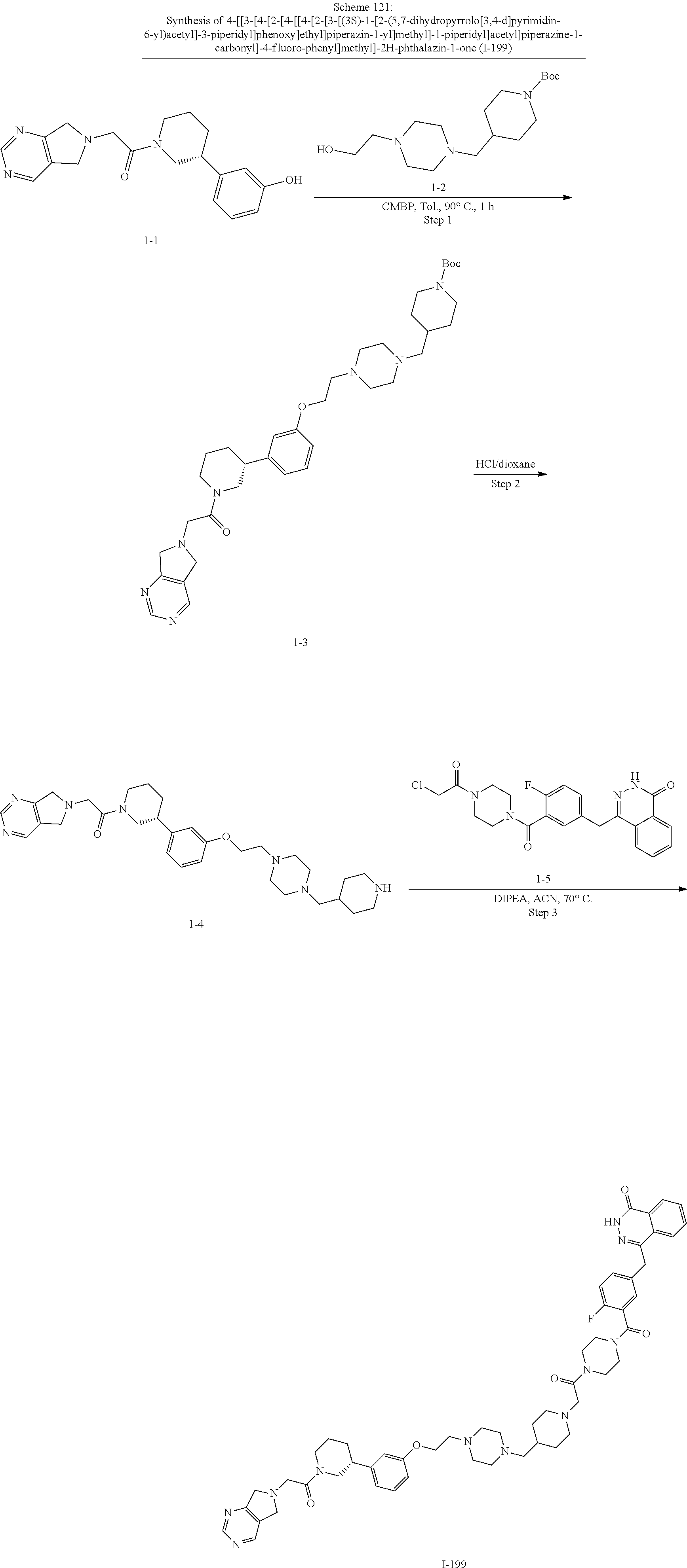

### Step 1: Synthesis of Intermediate 1-3

To a mixture of intermediate 1-1 (100.00 mg, 295.51 μmol, 1 eq) and intermediate 1-2 (96.77 mg, 295.51 μmol, 1 eq) in toluene (1 mL) was added 2-(tributyl-phosphanylidene) acetonitrile (213.96 mg, 886.52 μmol, 3 eq), then the mixture was stirred at 90° C. for 1 hour under N₂ atmosphere. The reaction mixture was concentrated under reduced pressure to give a residue. The crude product was purified by reversed-phase HPLC (0.1% FA condition). The eluent was concentrated under reduced pressure to remove MeCN and then lyophilized to give the intermediate 1-3 (100 mg, 154.36 μmol, 52.23% yield) as a yellow solid. LCMS (Method D): Rt=0.295 min, [M+H]⁺=648.4.

### Step 2: Synthesis of Intermediate 1-4

A mixture of intermediate 1-3 (100 mg, 154.36 μmol, 1 eq) in HCl/dioxane (1 mL) was stirred at 20° C. for 10 mins. The reaction mixture was concentrated under reduced pressure to give intermediate 1-4 (100 mg, crude, HCl salt) as a yellow solid.

### Step 3: Synthesis of I-199

To a mixture of intermediate 1-4 (100 mg, 171.18 μmol, 1 eq, HCl salt) in DMF (1 mL) was added intermediate 1-5 (75.81 mg, 171.18 μmol, 1 eq) and DIEA (110.62 mg, 855.88 μmol, 149.08 μL, 5 eq), then the mixture was stirred at 60° C. for 1 hour. The reaction mixture was quenched by with water (10 mL) and extracted with EA (3 mL*2). The combined organic layers were concentrated under reduced pressure to give a residue. The crude product was purified by reversed-phase HPLC (0.1% FA condition). The eluent was concentrated under reduced pressure to remove MeCN and then lyophilized to give I-199 as a yellow gum. LCMS (Method D): Rt=0.274 min, [M+H]⁺=954.6. SFC: Retention time: 5.879 min. ¹H NMR (400 MHz, METHANOL-d4) δ=8.99 (d, J=2.8 Hz, 1H), 8.64 (s, 1H), 8.37 (d, J=6.8 Hz, 3H), 7.99-7.93 (m, 1H), 7.91-7.80 (m, 2H), 7.51 (s, 1H), 7.41-7.34 (m, 1H), 7.28-7.14 (m, 2H), 6.94-6.77 (m, 3H), 4.62-4.50 (m, 1H), 4.39 (s, 2H), 4.27-4.10 (m, 6H), 4.02 (d, J=13.8 Hz, 1H), 3.94 (s, 1H), 3.90-3.63 (m, 6H), 3.56 (d, J=15.6 Hz, 2H), 3.47-3.32 (m, 5H), 3.22-2.98 (m, 7H), 2.86-2.63 (m, 8H), 2.48 (s, 2H), 2.02-1.76 (m, 6H), 1.70-1.43 (m, 3H). ¹⁹F NMR (376 MHz, METHANOL-d4) δ=−120.57-−120.84.

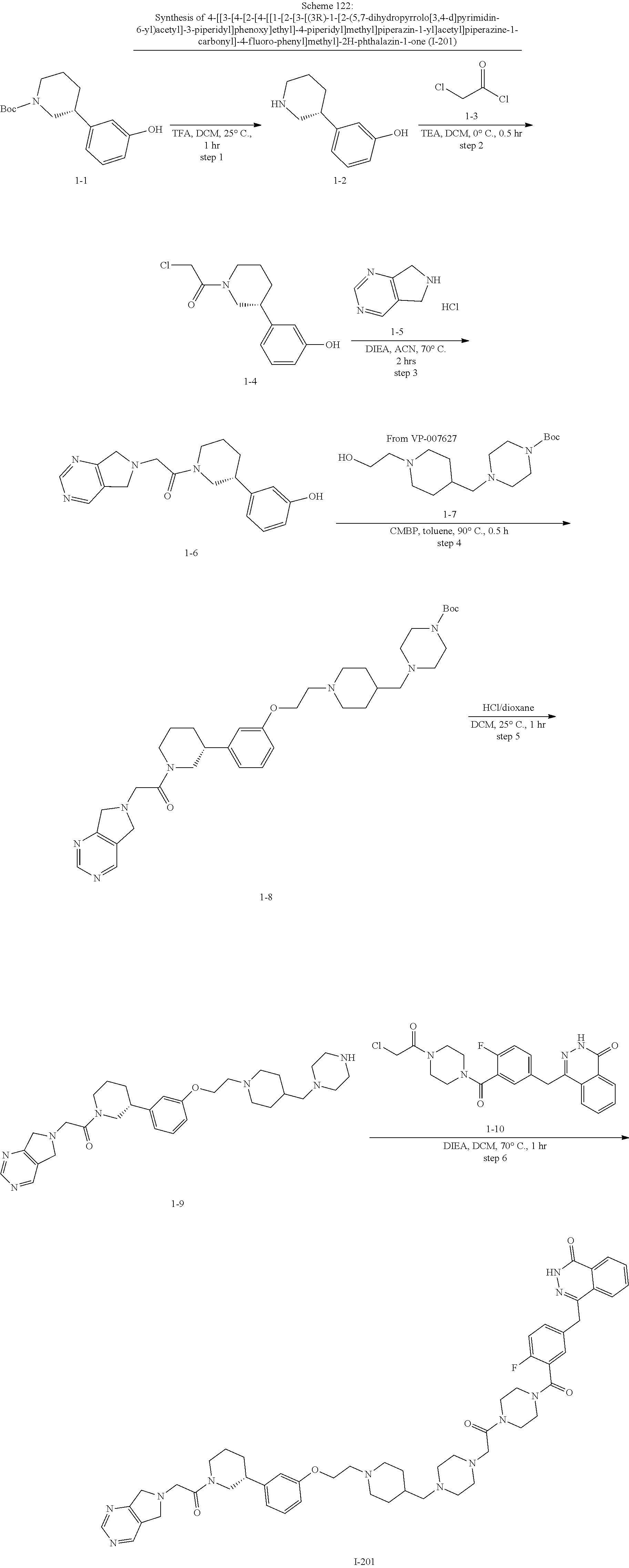

### Step 1: Synthesis of Intermediate 1-2

A solution of intermediate 1-1 (500 mg, 1.80 mmol, 1 eq) in CH₂Cl₂ (5 mL) and TFA (1 mL) was stirred at 25° C. for 1 hr. The mixture was concentrated under reduced pressure to give intermediate 1-2 (530 mg, crude, TFA salt) as yellow oil, which was used in the next step. LCMS (Method H): Rt: 0.345 min, (M+H)=690.5.

### Step 2: Synthesis of Intermediate 1-4

To a solution of intermediate 1-2 (530 mg, 1.82 mmol, 1 eq, TFA salt) in CH₂Cl₂ (5 mL) was added TEA (552.38 mg, 5.46 mmol, 759.81 μL, 3 eq). Then intermediate 1-3 (246.62 mg, 2.18 mmol, 173.92 L, 1.2 eq) was dropwise added into the mixture and stirred at 0° C. for 0.5 hr. The reaction mixture diluted with H₂O (5 mL) and extracted with CH₂Cl₂ (5 mL*2). The combined organic layers were dried over Na₂SO₄, filtered and concentrated under reduced pressure to give a residue. The residue was purified by flash silica gel chromatography (ISCO; 12 g SepaFlash Silica Flash Column, Eluent of 0˜40% Ethyl acetate/Petroleum ethergradient @50 mL/min). The eluent was concentrated under reduced pressure to give intermediate 1-4 (350 mg, 1.37 mmol, 75.05% yield, 99% purity) as a yellow solid. LCMS (Method D): Rt: 0.292 min, (M+H)=254.0. SFC: Rt: 1.578 min. ¹H NMR (400 MHz, METHANOL-d4) δ=7.18-7.08 (m, 1H), 6.80-6.61 (m, 3H), 4.59-4.47 (m, 1H), 4.36-4.28 (m, 1H), 4.28-4.13 (m, 1H), 4.02-3.89 (m, 1H), 3.25-3.10 (m, 1H), 2.81-2.54 (m, 2H), 2.07-1.96 (m, 1H), 1.94-1.82 (m, 1H), 1.82-1.52 (m, 2H).

### Step 3: Synthesis of Intermediate 1-6

To a solution of intermediate 1-5 (321.24 mg, 1.66 mmol, 1.2 eq) in ACN (3 mL) was added DIEA (534.85 mg, 4.14 mmol, 720.83 μL, 3 eq) and intermediate 1-4 (350 mg, 1.38 mmol, 1 eq). The mixture was stirred at 70° C. for 2 hours. The reaction mixture diluted with H₂O (5 mL) and extracted with EA (5 mL*2), the combined organic layers were washed with brine (1 mL*2), dried over Na₂SO₄, filtered and concentrated under reduced pressure to give a residue. The residue was purified by flash silica gel chromatography (ISCO; 12 g SepaFlash Silica Flash Column, Eluent of 0˜20% MeOH/CH₂Cl₂ @30 mL/min). The eluent was concentrated under reduced pressure to give intermediate 1-6 (320 mg, 899.28 μmol, 65.19% yield, 95.1%purity) as yellow oil. LCMS (Method D): Rt: 0.194 min, (M+H)=339.0. SFC: Rt: 1.209 min.

### Step 4: Synthesis of Intermediate 1-8

To a solution of intermediate 1-6 (100 mg, 295.51 μmol, 1 eq) and intermediate 1-7 (145.15 mg, 443.26 μmol, 1.5 eq) in toluene (1 mL) was added 2-(tributyl-phosphanylidene) acetonitrile (213.96 mg, 886.52 μmol, 3 eq) underN₂ atmosphere. The mixture was stirred at 90° C. for 0.5 hour. The reaction mixture diluted with H₂O (1.5 mL) and extracted with EA (1.5 mL*2). The combined organic layers were dried over Na₂SO₄, filtered and concentrated under reduced pressure to give a residue. The residue was purified by flash silica gel chromatography (ISCO; 12 g SepaFlash Silica Flash Column, Eluent of 0˜25% MeOH/CH₂Cl₂ @30 mL/min). The eluent was concentrated under reduced pressure to give intermediate 1-8 (140 mg, 213.29 μmol, 72.18% yield, 98.7% purity) as yellow oil. LCMS (Method D): Rt: 0.213 min, (M+H)=648.4. SFC: Rt: 1.130 min.

### Step 5: Synthesis of Intermediate 1-9

A solution of intermediate 1-8 (110 mg, 169.79 μmol, 1 eq) in CH₂Cl₂ (0.5 mL) and HCl/dioxane (2 M, 1.10 mL, 12.96 eq) was stirred at 25° C. for 1 hr. The mixture was concentrated under reduced pressure to give intermediate 1-9 (100 mg, crude, HCl salt) as a yellow oil and used in the next step. LCMS (Method D): Rt: 0.134 min, (M+H)=548.4.

### Step 6: Synthesis of I-201

To a solution of Intermediate 1-9 (70.00 mg, 119.82 μmol, 1 eq, HCl salt) and Intermediate 1-10 (53.07 mg, 119.82 μmol, 1 eq) in ACN (1 mL) was added DIEA (46.46 mg, 359.47 μmol, 62.61 μL, 3 eq). The mixture was stirred at 70° C. for 1 hr. The reaction mixture diluted with H₂O (1 mL) and extracted with EA (1.5 mL*2). The combined organic layers were dried over Na₂SO₄, filtered and concentrated under reduced pressure to give a residue. The residue was purified by prep-HPLC (column: Phenomenex luna C18 150*25 mm*10 um; mobile phase: [water (FA)-ACN]; gradient: 0%-26% B over 10 min). The eluent was concentrated under reduced pressure to remove ACN and then lyophilized to give I-201 (12.89 mg, 13.51 μmol, 11.27% yield, 100% purity) as yellow gum. LCMS (Method D): Rt: 0.278 min, (M+H)=954.4. SFC: Rt: 5.797 min. ¹H NMR (400 MHz, METHANOL-d4) δ=9.02-8.96 (m, 1H), 8.64 (s, 1H), 8.41-8.35 (m, 2H), 7.99-7.91 (m, 1H), 7.91-7.79 (m, 2H), 7.55-7.45 (m, 1H), 7.41-7.32 (m, 1H), 7.31-7.21 (m, 1H), 7.20-7.12 (m, 1H), 6.98-6.80 (m, 3H), 4.62-4.48 (m, 1H), 4.39 (s, 2H), 4.37-4.27 (m, 2H), 4.24-4.10 (m, 4H), 4.06-3.96 (m, 1H), 3.91-3.65 (m, 6H), 3.65-3.36 (m, 9H), 3.36-3.32 (m, 1H), 3.21-3.10 (m, 1H), 3.08-2.92 (m, 2H), 2.83-2.57 (m, 10H), 2.44 (s, 2H), 2.08-1.96 (m, 3H), 1.96-1.73 (m, 3H), 1.72-1.42 (m, 3H). ¹⁹F NMR (376 MHz, METHANOL-d4) δ=−120.779.

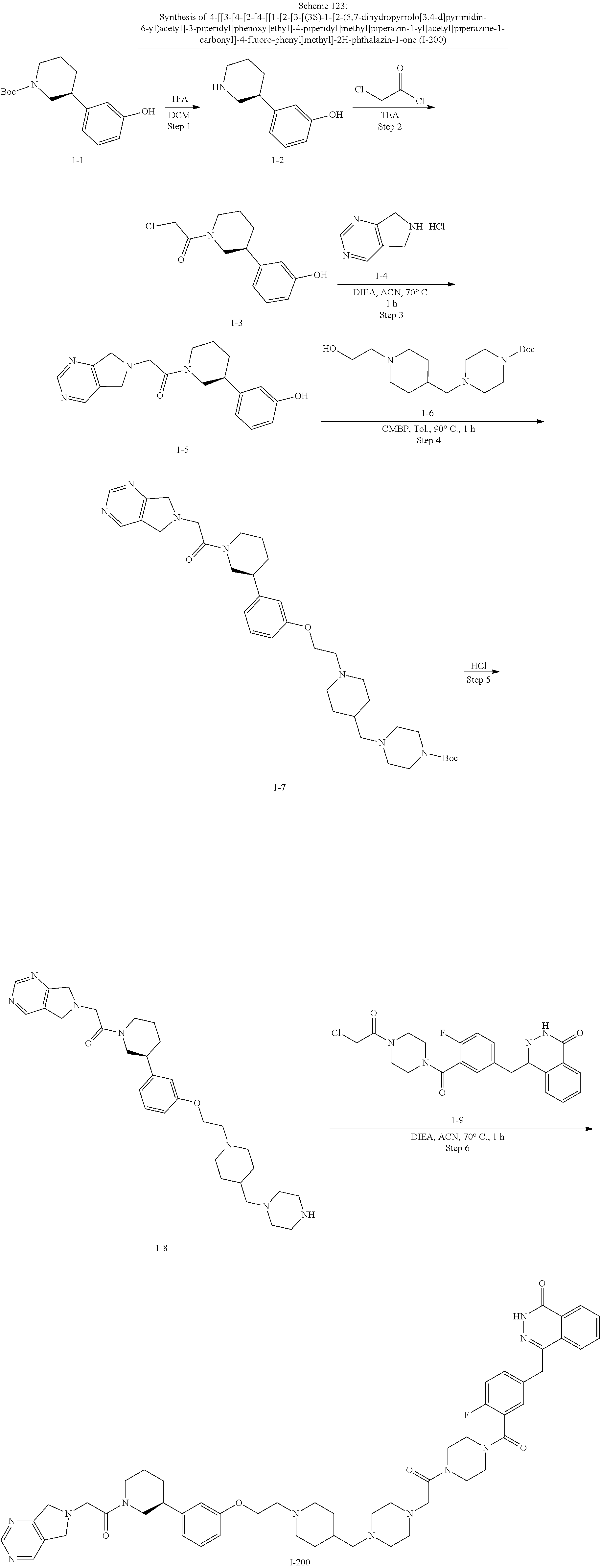

### Step 1: Synthesis of Intermediate 1-2

To a solution of intermediate 1-1 (1.7 g, 6.13 mmol, 1 eq) in DCM (5 mL) was added TFA (3.07 g, 26.93 mmol, 2.00 mL, 4.39 eq). The mixture was stirred at 25° C. for 2 hrs. The mixture was concentrated to give intermediate 1-2 (1.7 g, crude, TFA salt) as a yellow oil. LCMS (Method D): Rt: 0.138 min, (M+H)=178.1.

### Step 2: Synthesis of Intermediate 1-3

To a solution of intermediate 1-2 (1.7 g, 5.84 mmol, 1 eq, TFA salt) in DCM (10 mL) was added TEA (1.77 g, 17.51 mmol, 2.44 mL, 3 eq) at 0° C. Then 2-chloroacetyl chloride (791.04 mg, 7.00 mmol, 557.86 μL, 1.2 eq) was dropwise added into the mixture and stirred at 0° C. for 1 hr. The reaction mixture was diluted with water (30 mL) and extracted with DCM (10 mL×3). The combined organic layers were washed with brine (10 mL×3) and dried over sodium sulfate, filtered and concentrated under reduced pressure to give a residue. The residue was purified by flash silica gel chromatography (ISCO®; 20 g SepaFlash® Silica Flash Column, Eluent of 24-30% Ethylacetate/Petroleum ether gradient @70 mL/min). The eluent was concentrated under reduced pressure to give intermediate 1-3 (1.4 g, 5.31 mmol, 91.03% yield, 96.29% purity) as a white solid. LCMS (Method D): Rt: 0.350 min, (M+H)=254.0. SFC: Rt: 1.585 min. ¹H NMR (400 MHz, METHANOL-d4) δ=7.13-7.11 (m, 1H), 6.81-6.58 (m, 3H), 4.60-4.45 (m, 1H), 4.36-4.12 (m, 2H), 4.02-3.87 (m, 1H), 3.18-3.12 (m, 1H), 2.82-2.53 (m, 2H), 2.01 (d, J=13.6 Hz, 1H), 1.92-1.82 (m, 1H), 1.82-1.52 (m, 2H).

### Step 3: Synthesis of Intermediate 1-5

To a solution of Intermediate 1-3 (1.4 g, 5.52 mmol, 1 eq) and intermediate 1-5 (1.18 g, 6.07 mmol, 1.1 eq) in ACN (6 mL) was added DIEA (3.57 g, 27.59 mmol, 4.81 mL, 5 eq). The mixture was stirred at 70° C. for 2.5 hrs. The reaction mixture was diluted with water (60 mL) and extracted with ethyl acetate (20 mL×3). The combined organic layers were washed with brine (20 mL×3) and dried over sodium sulfate, filtered and concentrated under reduced pressure to give a residue. The residue was purified by flash silica gel chromatography (ISCO; 20 g SepaFlash Silica Flash Column, Eluent of 0-20% Methanol/Ethyl acetate gradient @70 mL/min). The eluent was concentrated under reduced pressure to give intermediate 1-5 (1.6 g, 4.36 mmol, 78.98% yield, 92.17% purity) as a brown gum. LCMS (Method D): Rt: 0.224 min, (M+H)=339.1. ¹H NMR (400 MHz, CHLOROFORM-d) δ=9.05 (s, 1H), 8.55 (s, 1H), 7.15 (t, J=7.6 Hz, 1H), 6.78-6.65 (m, 3H), 4.64 (d, J=10.0 Hz, 1H), 4.26-4.15 (m, 4H), 3.93 (d, J=12.4 Hz, 1H), 3.71 (s, 1H), 3.15-3.08 (m, 1H), 2.72-2.56 (m, 2H), 2.09-2.00 (m, 1H), 1.85 (d, J=13.2 Hz, 2H), 1.71 (d, J=12.4 Hz, 1H), 1.64-1.53 (m, 1H).

### Step 4: Synthesis of Intermediate 1-7

To a solution of intermediate 1-5 (100 mg, 295.51 μmol, 1 eq) and intermediate 1-6 (193.53 mg, 591.01 μmol, 2 eq) in toluene (0.5 mL) was added 2-(tributyl-phosphanylidene) acetonitrile (213.96 mg, 886.52 μmol, 3 eq) under N₂ atmosphere. The mixture was stirred at 90° C. for 1 hr under N₂ atmosphere. The reaction mixture was diluted with water (30 mL) and extracted with ethyl acetate (10 mL×3). The combined organic layers were washed with brine (10 mL×3) and dried over sodium sulfate, filtered and concentrated under reduced pressure to give a residue. The crude product was purified by reversed-phase HPLC (0.10% FA condition). The eluent was concentrated under reduced pressure to remove ACN and then lyophilized to give intermediate 1-7 (120 mg, 184.34 μmol, 62.38% yield, 99.52% purity) as a brown oil. LCMS: Rt: 0.254 min, (M+H)=648.4. SFC: Rt: 1.120 min. ¹H NMR (400 MHz, CHLOROFORM-d) δ=9.06 (s, 1H), 8.57 (s, 1H), 8.37 (s, 1H), 7.27-7.20 (m, 1H), 6.91-6.65 (m, 2H), 4.77-4.60 (m, 1H), 4.54-4.13 (m, 10H), 3.95 (d, J=12.0 Hz, 1H), 3.80-3.69 (m, 1H), 3.69-3.51 (m, 2H), 3.42 (s, 4H), 3.37-3.18 (m, 2H), 3.17-3.02 (m, 1H), 2.68-2.62 (m, 3H), 2.38 (s, 4H), 2.26 (d, J=6.0 Hz, 2H), 2.15-1.79 (m, 4H), 1.76-1.54 (m, 4H), 1.46 (s, 9H).

### Step 5: Synthesis of Intermediate 1-8

To a solution of intermediate 1-7 (120 mg, 185.23 μmol, 1 eq) in DCM (0.5 mL) was added HCl/dioxane (2 M, 0.5 mL). The mixture was stirred at 25° C. for 0.5 hr. The mixture was concentrated to give intermediate 1-8 (110 mg, crude, HCl salt) as a yellow solid. LCMS (Method D): Rt: 0.162 min, (M+H)=548.3.

### Step 6: Synthesis of I-200

To a solution of Intermediate 1-8 (100 mg, 171.18 μmol, 1 eq, HCl salt) in ACN (0.2 mL) was added DIEA (66.37 mg, 513.53 μmol, 89.45 μL, 3 eq). Then intermediate 1-9 (75.81 mg, 171.18 μmol, 1 eq) was added into the mixture and it was stirred at 70° C. for 0.5 hr. The reaction mixture was diluted with water (10 mL) and extracted with ethyl acetate (3 mL×3). The combined organic layers were washed with brine (3 mL×3) and dried over sodium sulfate, filtered and concentrated under reduced pressure to give a residue. The residue was purified by prep-HPLC (column: Phenomenex luna C18 150*25 mm*10 um; mobile phase: [water (FA)-ACN]; gradient: 0%-21% B over 10 min). The eluent was concentrated under reduced pressure to give the product. The eluent was concentrated under reduced pressure to remove ACN and then lyophilized to give I-200 (74.61 mg, 73.84 μmol, 43.13% yield, 98.98% purity, FA salt) as a yellow gum. LCMS (Method D): Rt: 0.305 min, (M+H)=954.5. SFC: Rt: 4.739 min. ¹H NMR (400 MHz, METHANOL-d4) δ=8.99 (d, J=4.0 Hz, 1H), 8.64 (s, 1H), 8.39-8.31 (m, 3H), 8.01-7.92 (m, 1H), 7.91-7.80 (m, 2H), 7.56-7.45 (m, 1H), 7.37-7.35 (m, 1H), 7.31-7.21 (m, 1H), 7.17-7.14 (m, 1H), 6.98-6.81 (m, 3H), 4.64-4.47 (m, 1H), 4.41-4.28 (m, 4H), 4.25-4.09 (m, 4H), 4.02 (d, J=12.8 Hz, 1H), 3.92-3.71 (m, 4H), 3.70-3.55 (m, 6H), 3.52 (s, 5H), 3.35 (s, 1H), 3.15-3.08 (m, 1H), 3.06 (s, 2H), 2.91-2.63 (m, 10H), 2.51 (s, 2H), 2.08-1.94 (m, 4H), 1.91-1.75 (m, 2H), 1.73-1.46 (m, 3H) ¹⁹F NMR (400 MHz, METHAN OL-d4) δ=−120.711.

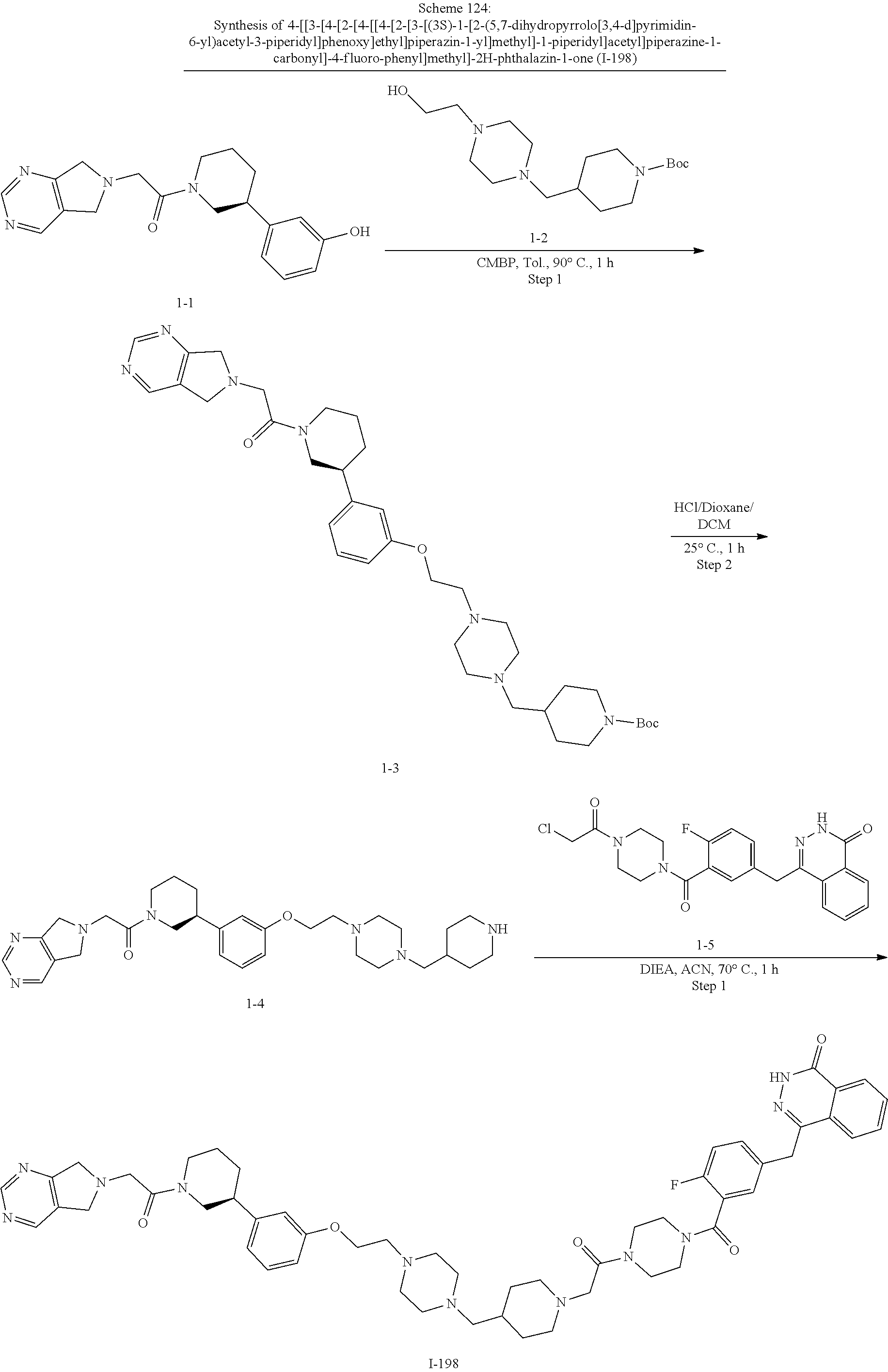 [SMILES: O=C(CN1Cc2cncnc2C1)N1CCC[C@@H](c2cccc(O)c2)C1 ; CC(C)(C)OC(=O)N1CCC(CN2CCN(CCO)CC2)CC1 ; CC(C)(C)OC(=O)N1CCC(CN2CCN(CCOc3cccc([C@@H]4CCCN(C(=O)CN5Cc6cncnc6C5)C4)c3)CC2)CC1 ; O=C(CN1Cc2cncnc2C1)N1CCC[C@@H](c2cccc(OCCN3CCN(CC4CCNCC4)CC3)c2)C1 ; O=C(CCl)N1CCN(C(=O)c2cc(Cc3n[nH]c(=O)c4ccccc34)ccc2F)CC1 ; O=C(CN1CCC(CN2CCN(CCOc3cccc([C@@H]4CCCN(C(=O)CN5Cc6cncnc6C5)C4)c3)CC2)CC1)N1CCN(C(=O)c2cc(Cc3n[nH]c(=O)c4ccccc34)ccc2F)CC1]

### Step 1: Synthesis of Intermediate 1-3

To a solution of intermediate 1-1 (100 mg, 295.51 μmol, 1 eq) and intermediate 1-2 (193.53 mg, 591.01 μmol, 2 eq) in toluene (1 mL) was added 2-(tributyl-phosphanylidene) acetonitrile (213.96 mg, 886.52 μmol, 3 eq). The mixture was stirred at 90° C. for 1 hr. The reaction mixture was diluted with water (20 mL) and extracted with ethyl acetate (7 mL×3). The combined organic layers were washed with brine (7 mL×3) and dried over sodium sulfate, filtered and concentrated under reduced pressure to give a residue. The crude product was purified by reversed-phase HPLC (0.1% FA condition). The eluent was concentrated under reduced pressure to remove ACN and then lyophilized to give intermediate 1-3 (160 mg, 221.85 μmol, 75.08% yield, 89.83% purity) as a brown oil. LCMS (Method D): Rt: 0.288 min, (M+H)=648.6. SFC: Rt: 1.168 min. ¹H NMR (400 MHz, CHLOROFORM-d) δ=9.05 (s, 1H), 8.56 (s, 1H), 8.35 (s, 1H), 7.23 (t, J=7.6 Hz, 1H), 6.88-6.68 (m, 2H), 5.50-5.11 (m, 4H), 4.77-4.60 (m, 1H), 4.25-4.16 (m, 4H), 4.09 (s, 2H), 3.99-3.87 (m, 1H), 3.82-3.55 (m, 2H), 3.05 (q, J=5.2 Hz, 2H), 3.00-2.86 (m, 3H), 2.82-2.56 (m, 8H), 2.42-2.26 (m, 2H), 2.06 (s, 1H), 1.87 (d, J=12.4 Hz, 1H), 1.81-1.53 (m, 5H), 1.45 (s, 9H), 1.19-0.96 (m, 2H).

### Step 2: Synthesis of Intermediate 1-4

To a solution of intermediate 1-3 (160 mg, 246.97 μmol, 1 eq) in DCM (0.5 mL) was added HCl/dioxane (2 M, 0.5 mL, 4.05 eq). The mixture was stirred at 25° C. for 0.5 hr. The mixture was concentrated to give intermediate 1-4 (150 mg, crude, HCl salt) as a yellow solid. LCMS (Method D): Rt: 0.200 min, (M+H)=548.4.

### Step 3: Synthesis of I-198

To a solution of intermediate 1-4 (70 mg, 119.82 μmol, 1 eq, HCl salt) and intermediate 1-5 (53.07 mg, 119.82 μmol, 1 eq) in ACN (0.5 mL) was added DIEA (46.46 mg, 359.47 μmol, 62.61 μL, 3 eq). The mixture was stirred at 70° C. for 1 hr. The reaction mixture was diluted with water (10 mL) and extracted with ethyl acetate (3 mL×3). The combined organic layers were washed with brine (3 mL×3) and dried over sodium sulfate, filtered and concentrated under reduced pressure to give a residue. The residue was purified by prep-HPLC (column: Phenomenex luna C18 150*25 mm*10 um; mobile phase: [water (FA)-ACN]; gradient: 0%-23% B over 10 min). The eluent was concentrated under reduced pressure to remove ACN and then lyophilized to give I-198 (15.13 mg, 14.73 μmol, 12.30% yield, 97.40% purity, FA salt) as a white solid. LCMS (Method D): Rt: 0.315 min, (M+H)=954.5. SFC: Rt: 4.823 min. ¹H NMR (400 MHz, METHANOL-d4) δ=8.98 (d, J=3.2 Hz, 1H), 8.64 (s, 1H), 8.42 (s, 2H), 8.35 (d, J=7.6 Hz, 1H), 7.98-7.91 (m, 1H), 7.84-7.82 (m, 2H), 7.50-7.45 (m, 1H), 7.41-7.34 (m, 1H), 7.29-7.20 (m, 1H), 7.16-7.14 (m, H), 6.96-6.76 (m, 3H), 4.63-4.48 (m, 1H), 4.38 (s, 2H), 4.28 (s, 1H), 4.22 (s, 2H), 4.20-4.10 (m, 3H), 4.09-3.95 (m, 3H), 3.93-3.72 (m, 4H), 3.68 (s, 1H), 3.55 (d, J=4.4 Hz, 2H), 3.51-3.32 (m, 5H), 3.30-3.01 (m, 7H), 2.95-2.61 (m, 8H), 2.50 (s, 2H), 2.06-1.94 (m, 3H), 1.93-1.72 (m, 3H), 1.71-1.44 (m, 3H). ¹⁹F NMR (400 MHz, METHAN OL-d4) δ=−120.711.

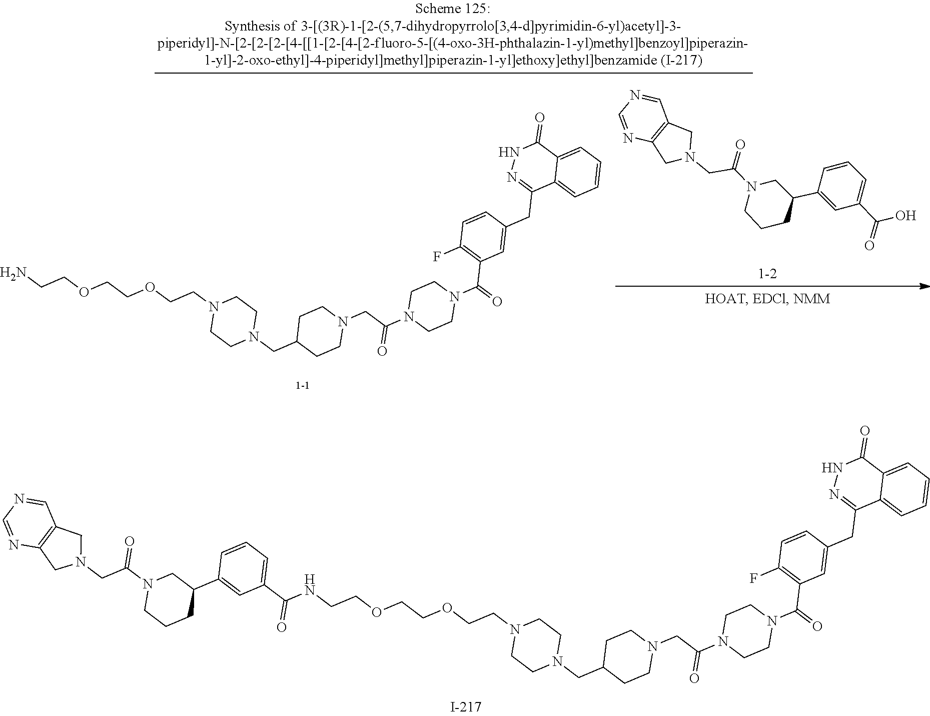

To a solution of Intermediate 1-2 (46.45 mg, 126.76 μmol, 1.2 eq) in DMF (0.8 mL) was added NMM (53.42 mg, 528.17 μmol, 58.07 μL, 5 eq), EDCI (101.25 mg, 528.17 μmol, 5 eq), HOAt (28.76 mg, 211.27 μmol, 29.55 μL, 2 eq), and Intermediate 1-1 (80 mg, 105.63 μmol, 1 eq, HCl salt). The mixture was stirred at 25° C. for 1 h. The reaction mixture was filtered to give the filtrate. The crude was purified by prep-HPLC (column: Phenomenex luna C18 150*25 mm*10 um; mobile phase: [water (FA)-ACN]; gradient: 0%-24% B over 10 min) the eluent was concentrated to remove ACN and lyophilized to give I-217 (12 mg, 11.19 μmol, 10.59% yield, 99.689% purity) as a yellow gum. LCMS (Method D): Retention time: 0.263 min, (M+H)=1069.4. SFC: Retention time: 2.276 min. ¹H NMR (400 MHz, METHANOL-d4) δ=8.98 (s, 1H), 8.64 (s, 1H), 8.44-8.34 (m, 3H), 7.98-7.92 (m, 1H), 7.90-7.80 (m, 2H), 7.78-7.66 (m, 2H), 7.50 (d, J=6.4 Hz, 1H), 7.46-7.41 (m, 1H), 7.39-7.33 (m, 1H), 7.17 (t, J=8.8 Hz, 1H), 4.58 (d, J=8.8 Hz, 1H), 4.39 (s, 2H), 4.23-4.11 (m, 4H), 4.05 (d, J=13.2 Hz, 1H), 3.88 (s, 1H), 3.84 (d, J=3.6 Hz, 1H), 3.80 (s, 2H), 3.76 (t, J=4.4 Hz, 3H), 3.67 (s, 8H), 3.59 (d, J=4.8 Hz, 3H), 3.53 (s, 1H), 3.43 (s, 1H), 3.39-3.32 (m, 3H), 3.18 (d, J=12.5 Hz, 1H), 3.09 (d, J=4.4 Hz, 6H), 2.90-2.81 (m, 1H), 2.77 (d, J=6.0 Hz, 1H), 2.71 (d, J=10.0 Hz, 6H), 2.41-2.30 (m, 2H), 2.08-2.02 (m, 1H), 1.89 (d, J=8.8 Hz, 4H), 1.80-1.72 (m, 1H), 1.71-1.60 (m, 1H), 1.50-1.38 (m, 2H), 1.38-1.23 (m, 1H). ¹⁹F NMR (376 MHz, METHANOL-d4) δ=−120.749.

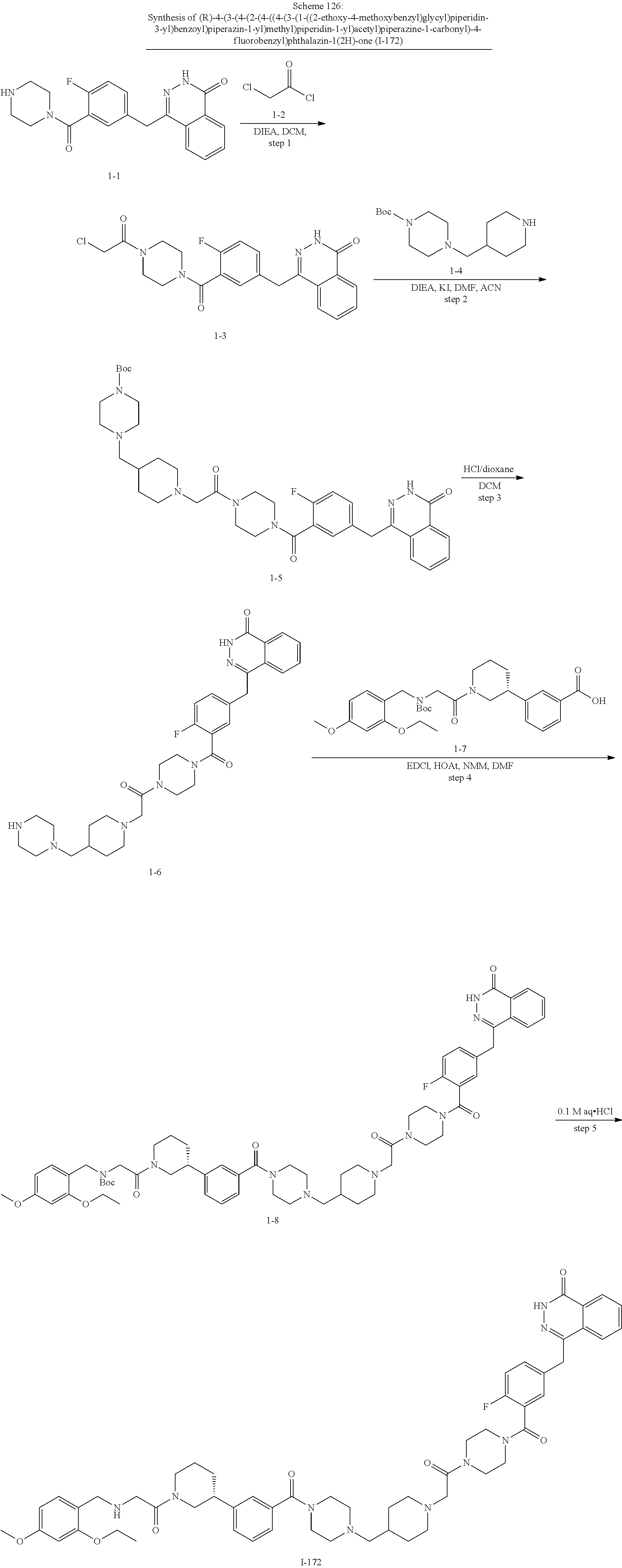

### Step 1: Synthesis of Intermediate 1-3

To a solution of Intermediate 1-1 (12 g, 32.75 mmol, 1 eq) in DCM (120 mL) was added DIEA (12.70 g, 98.26 mmol, 17.11 mL, 3 eq) and Intermediate 1-2 (4.44 g, 39.30 mmol, 3.13 mL, 1.2 eq) at 0° C. The mixture was stirred at 25° C. for 2 hr. The reaction mixture was washed with water (100 mL) and extracted with DCM (50 mL*2). The combined organic phase was washed with brine (100 mL), dried with anhydrous Na₂SO₄, filtered and concentrated in vacuum to give Intermediate 1-3 (13 g, 29.35 mmol, 89.62% yield) as a brown solid. LCMS (Method D): Rt=0.421 min, [M+H]⁺=443.0.

### Step 2: Synthesis of Intermediate 1-5

To a solution of Intermediate 1-4 (1.87 g, 6.60 mmol, 1.5 eq) in ACN (10 mL) and DMF (12 mL) was added DIEA (2.28 g, 17.61 mmol, 3.07 mL, 4 eq) under N₂, the reaction mixture was stirred at 25° C. for 0.5 h, then KI (365.46 mg, 2.20 mmol, 0.5 eq) and Intermediate 1-3 (1.95 g, 4.40 mmol, 1 eq) were added to the mixture, and the reaction mixture was stirred at 60° C. for 12 hrs. The reaction mixture was quenched by addition of H₂O (20 mL), and then diluted with EA (20 mL) and extracted with EA (40 mL*3). The combined organic layers were washed with brine (40 mL*3), dried over anhydrous Na₂SO₄, filtered and concentrated under reduced pressure to give a residue. The residue was purified by reversed phase HPLC (0.1% NH₃·H₂O condition), then concentrated to remove organic solvents and lyophilized to give Intermediate 1-5 (2.3 g, 3.20 mmol, 72.70% yield, 96% purity) as a yellow solid. LCMS (Method E): Rt=0.390 min, [M+H]⁺=690.4.

### Step 3: Synthesis of Intermediate 1-6

To a solution of Intermediate 1-5 (2.3 g, 3.33 mmol, 1 eq) in DCM (23 mL) was added HCl/dioxane (4 M, 23 mL, 27.59 eq). The mixture was stirred at 25° C. for 1 hr. The reaction mixture was concentrated under reduced pressure to give Intermediate 1-6 (2.1 g, 3.32 mmol, 99.5% yield, 99% purity, HCl salt) as a brown solid. LCMS (Method E): Rt=0.359 min, [M+H]⁺=590.3. ¹H NMR (400 MHz, DMSO-d₆) δ=12.67-12.57 (m, 1H), 10.28-10.19 (m, 1H), 8.25 (d, J=8.0 Hz, 1H), 8.01-7.78 (m, 3H), 7.51-7.32 (m, 2H), 7.26-7.24 (m, 1H), 4.56-4.30 (m, 6H), 3.81-3.57 (m, 6H), 3.53-3.49 (m, 3H), 3.46-3.19 (m, 8H), 3.12-2.98 (m, 3H), 2.16 (d, J=11.2 Hz, 2H), 1.99 (d, J=13.2 Hz, 1H), 1.68-1.20 (m, 2H). ¹⁹FNMR (377 MHz, DMSO-d₆), δ=−119.7.

### Step 4: Synthesis of Intermediate 1-8

To a solution of Intermediate 1-6 (71.34 mg, 113.93 μmol, 1.2 eq, HCl salt) and Intermediate 1-7 (50 mg, 94.95 μmol, 1 eq) in DMF (1 mL) was added EDCI (54.60 mg, 284.84 μmol, 3 eq), HOAt (12.92 mg, 94.95 μmol, 1 eq) and NMM (96.03 mg, 949.45 μmol, 104.38 μL, 10 eq). The mixture was stirred at 25° C. for 2 hrs. The reaction mixture was quenched by addition of H₂O (1 mL), and then diluted with EA (1 mL) and extracted with EA (2 mL*3). The combined organic layers were washed with brine (2 mL*3), dried over anhydrous Na₂SO₄, filtered and concentrated under reduced pressure to give a residue. The residue was purified by reversed phase HPLC (0.1% FA condition), and then concentrated to remove organic solvents and lyophilized to give Intermediate 1-8 (80 mg, 72.11 μmol, 75.95% yield, 99% purity) as a white solid. LCMS (Method E): Rt=0.473 min, [M+H]⁺=1098.6. SFC: Rt=3.780 min, ee value=99.314%.

### Step 5: Synthesis of I-172

A solution of Intermediate 1-8 (60 mg, 54.63 μmol, 1 eq) in aq. HCl (0.1 M, 1.64 mL, 3 eq) was stirred at 100° C. for 0.5 hr. The reaction mixture was adjusted to pH=8 with NH₃·H₂O, and then concentrated under reduced pressure to give a residue. The residue was purified by prep-HPLC (column: Waters Xbridge 150*25 mm*5 um; mobile phase: [water (ammonia hydroxide v/v)-ACN]; gradient: 33%-63% B over 10 min), then concentrated to remove organic solvents and lyophilized to give I-172 (25.21 mg, 24.50 μmol, 44.8% yield, 97% purity) as a white solid. LCMS (Method E): Rt=0.405 min, [M+H]⁺=998.8. SFC: Rt=5.610 min, ee value>99%. ¹H NMR (400 MHz, METHANOL-d₄) δ=8.40-8.34 (m, 1H), 7.97-7.92 (m, 1H), 7.90-7.79 (m, 2H), 7.48 (br s, 1H), 7.44-7.34 (m, 3H), 7.32-7.24 (m, 2H), 7.16-7.12 (m, 2H), 6.53-6.42 (m, 2H), 4.56-4.46 (m, 1H), 4.38 (s, 2H), 4.10-4.00 (m, 2H), 3.84 (d, J=2.4 Hz, 1H), 3.81-3.62 (m, 12H), 3.59-3.34 (m, 7H), 3.25-3.16 (m, 2H), 3.15-3.02 (m, 1H), 2.95-2.83 (m, 2H), 2.78-2.59 (m, 2H), 2.56-2.33 (m, 4H), 2.25-2.21 (m, 2H), 2.12-1.96 (m, 3H), 1.87-1.71 (m, 4H), 1.64-1.49 (m, 2H), 1.45-1.39 (m, 3H), 1.32-1.15 (m, 2H). ¹⁹F NMR (377 MHz, METHANOL-d₄) δ=−120.8.

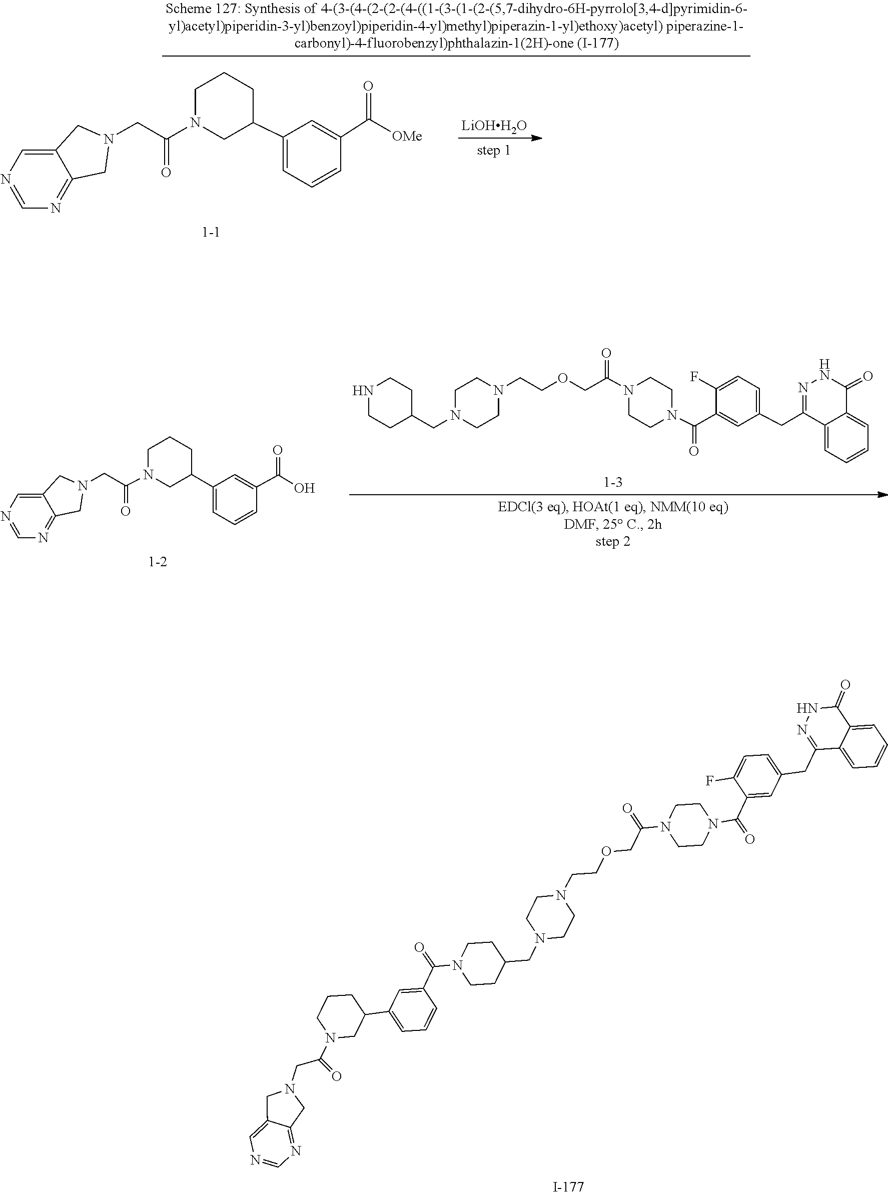 [SMILES: COC(=O)c1cccc(C2CCCN(C(=O)CN3Cc4cncnc4C3)C2)c1 ; "LiOH·H2O" ; O=C(O)c1cccc(C2CCCN(C(=O)CN3Cc4cncnc4C3)C2)c1 ; O=C(COCCN1CCN(CC2CCNCC2)CC1)N1CCN(C(=O)c2cc(Cc3n[nH]c(=O)c4ccccc34)ccc2F)CC1 ; O=C(COCCN1CCN(CC2CCN(C(=O)c3cccc(C4CCCN(C(=O)CN5Cc6cncnc6C5)C4)c3)CC2)CC1)N1CCN(C(=O)c2cc(Cc3n[nH]c(=O)c4ccccc34)ccc2F)CC1]

### Step 1: Synthesis of Intermediate 1-2

To a solution of Intermediate 1-1 (260 mg, 683.42 μmol, 1 eq) in MeOH (2 mL), THF (2 mL) and H₂O (0.5 mL) was added LiOH·H₂O (143.39 mg, 3.42 mmol, 5 eq). The mixture was stirred at 25° C. for 1 hr. The reaction mixture was concentrated under reduced pressure to give a residue. The residue was purified by reversed phase (NH₃·H₂O condition), then concentrated to remove ACN and lyophilized to give Intermediate 1-2 (160 mg, 436.67 μmol, 63.89% yield) as a yellow solid. LCMS (Method C): Rt=0.385 min, [M+H]⁺=367.2.

### Step 2: Synthesis of I-177

To a solution of Intermediate 1-2 (50 mg, 136.46 μmol, 1 eq) and Intermediate 1-3 (86.48 mg, 136.46 μmol, 1 eq) in DMF (0.5 mL) was added EDCI (78.48 mg, 409.37 μmol, 3 eq), HOAt (18.57 mg, 136.46 μmol, 19.09 μL, 1 eq) and NMM (138.02 mg, 1.36 mmol, 150.03 μL, 10 eq). The mixture was stirred at 25° C. for 2 hrs. The reaction mixture was quenched by addition of H₂O (0.5 mL), and then diluted with EA (0.5 mL) and extracted with EA (1 mL*3). The combined organic layers were washed with brine (1 mL*3), dried over anhydrous Na₂SO₄, filtered and concentrated under reduced pressure to give a residue. The residue was purified by prep-HPLC (column: Waters Xbridge 150*25 mm*5 um; mobile phase: [water (ammonia hydroxide v/v)-ACN]; gradient: 8%-38% B over 10 min), then concentrated to remove organic solvents and lyophilized to give I-177 (13.72 mg, 12.99 μmol, 9.52% yield, 93% purity) as a white solid. LCMS (Method G): Rt=0.520 min, [M+H]⁺=982.7. SFC: Rt=0.837 min, 1.231 min. ¹H NMR (400 MHz, METHANOL-d₄) δ=8.98 (s, 1H), 8.63 (s, 1H), 8.39-8.35 (m, 1H), 7.98-7.79 (m, 3H), 7.44 (s, 5H), 7.24 (d, J=7.2 Hz, 1H), 7.18-7.13 (m, 1H), 4.62-4.52 (m, 3H), 4.38 (s, 2H), 4.26-4.10 (m, 6H), 4.07-4.00 (m, 1H), 3.91-3.72 (m, 4H), 3.70-3.59 (m, 5H), 3.54-3.45 (m, 2H), 3.22-3.07 (m, 2H), 2.87-2.70 (m, 3H), 2.67-2.30 (m, 9H), 2.27-2.00 (m, 4H), 1.96-1.52 (m, 7H), 1.26-1.03 (m, 2H). ¹⁹F NMR (377 MHz, METHANOL-d₄), δ=−120.7.

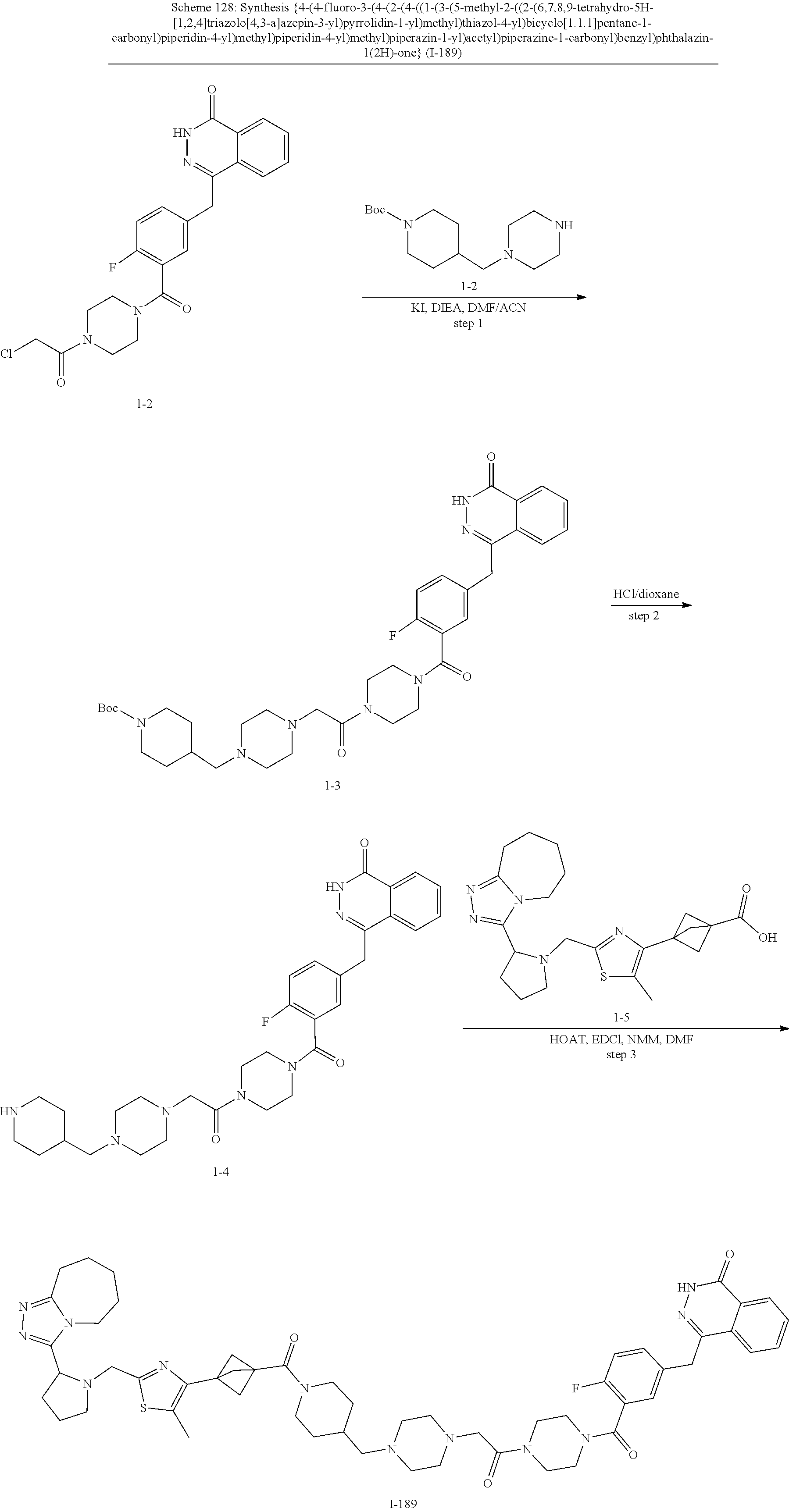

### Step 1: Synthesis of Intermediate 1-3

To a solution of Intermediate 1-1 (0.7 g, 1.58 mmol, 1 eq) in DMF (3 mL) and ACN (3 mL) was added KI (262.38 mg, 1.58 mmol, 1 eq), DIEA (817.13 mg, 6.32 mmol, 1.10 mL, 4 eq) and Intermediate 1-2 (492.75 mg, 1.74 mmol, 1.1 eq) at 20° C. The mixture was stirred at 60° C. for 0.5 h. The reaction mixture was quenched with H₂O (10 mL) and extracted with EA (8 ml*3), the combined organic layer was washed with brine (8 mL*3), dried over with Na₂SO₄, then filtered and concentrated under reduced pressure to give a residue. The residue was purified by reversed-phase HPLC (0.1% FA condition) and then reversed-phase HPLC (0.1% NH₃·H₂O condition). The eluent was concentrated under reduced pressure to remove ACN, then lyophilized to afford Intermediate 1-3 (330 mg, 425.76 μmol, 26.94% yield, 89% purity) as yellow solid. LCMS (Method E): Retention time: 0.436 min, (M+H)⁺=690.4.

### Step 2: Synthesis of Intermediate 1-4

To a solution of Intermediate 1-3 (50 mg, 72.48 μmol, 1 eq) in dioxane (0.5 mL) was added HCl/dioxane (2 M, 0.5 mL, 13.80 eq). The mixture was stirred at 25° C. for 1 h. The mixture was concentrated under vacuum to give Intermediate 1-4 (50 mg, crude, HCl salt) as a yellow solid. LCMS (Method E): Retention time: 0.350 min, (M+H)⁺=590.3.

### Step 3: Synthesis of I-189

To a solution of Intermediate 1-5 (30 mg, 70.17 μmol, 1 eq), HOAt (9.55 mg, 70.17 μmol, 9.82 L, 1 eq), EDCI (26.90 mg, 140.33 μmol, 2 eq) and NMM (35.49 mg, 350.83 μmol, 38.57 μL, 5 eq) in DMF (0.5 mL) was added Intermediate 1-4 (41.38 mg, 66.08 μmol, 0.94 eq, HCl salt). The mixture was stirred at 25° C. for 1 h. The mixture was filtered. The filtrate was purified by Prep-HPLC (column: Waters Xbridge 150*25 mm*5 um; mobile phase: [water (NH₄HCO₃)-ACN]; gradient: 20%-50% B over 15 min). The eluent was concentrated under reduced pressure to remove ACN, then lyophilized to afford I-189 (42.08 mg, 41.99 μmol, 59.85% yield, 99.72% purity) as a white solid. LCMS (Method G): Rt=0.559 min, (M+H)⁺=999.6. SFC: Rt=6.377 min, Rt=9.515 min. ¹H NMR (400 MHz, DMSO-d6) δ=12.60 (s, 1H), 8.27 (d, J=7.6 Hz, 1H), 8.02-7.96 (m, 1H), 7.92-7.81 (m, 2H), 7.44 (br s, 1H), 7.38 (brt, J=6.4 Hz, 1H), 7.24 (br t, J=8.7 Hz, 1H), 4.34-4.09 (m, 6H), 4.01-3.94 (m, 1H), 3.75 (d, J=14.9 Hz, 1H), 3.68-3.41 (m, 7H), 3.24-2.94 (m, 7H), 2.91-2.81 (m, 2H), 2.47-2.26 (m, 18H), 2.19-2.06 (m, 3H), 1.89-1.54 (m, 12H), 0.97-0.81 (m, 2H). ¹⁹F NMR (377 MHz, DMSO-d6) δ=−119.7.

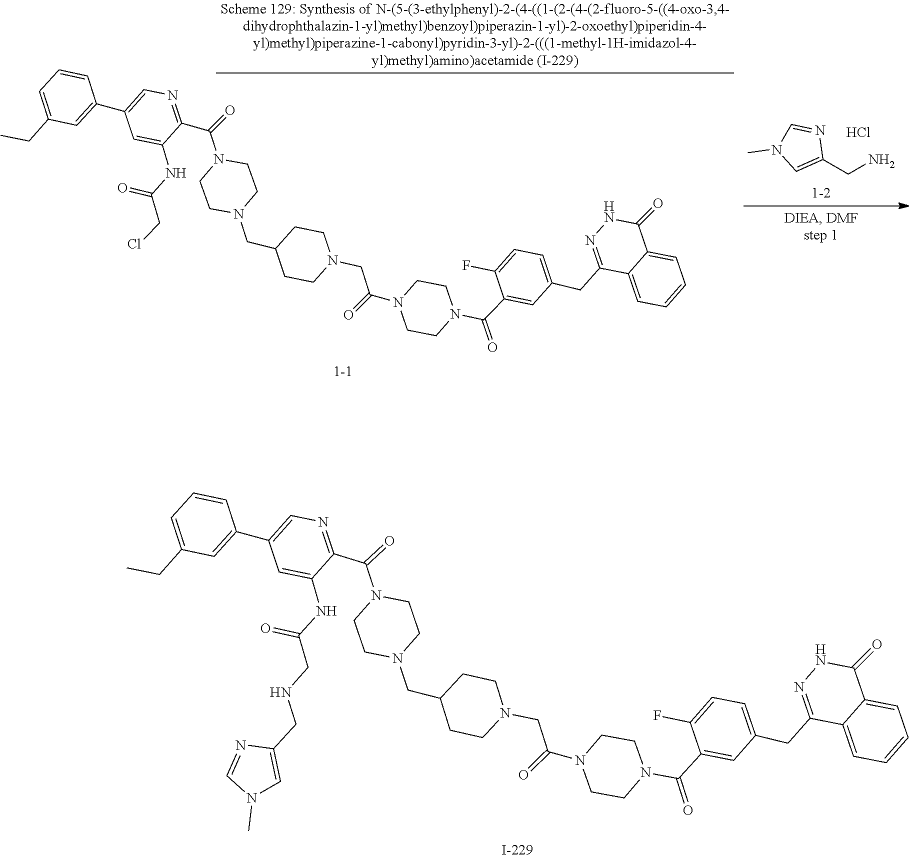

To a solution of Intermediate 1-1 (80.09 mg, 89.94 μmol, 1 eq) in DMF (1.5 mL) was added DIEA (58.12 mg, 449.71 μmol, 78.33 μL, 5 eq) and Intermediate 1-2 (19.91 mg, 134.91 μmol, 1.5 eq, HCl salt). The mixture was stirred at 100° C. for 3 hrs. The mixture was filtered. The filtrate was purified by Prep-HPLC (column: Phenomenex luna C18 150*25 mm*10 um; mobile phase: [water (FA)-ACN]; gradient: 2%-32% B over 9 min) and prep-HPLC (column: Waters Xbridge 150*25 mm*5 um; mobile phase: [water (NH₄HCO₃)-ACN]; gradient: 22%-52% B over 9 min). The eluent was concentrated under reduced pressure to remove ACN, then lyophilized to afford I-229 (11.28 mg, 11.66 μmol, 12.96% yield, 99.7% purity) as an off-white solid. LCMS (Method E): Rt=0.405 min, [M+H]⁺=965.5. ¹H NMR (400 MHz, METHANOL-d₄) δ=8.77 (s, 1H), 8.62 (d, J=2.0 Hz, 1H), 8.38 (m, 1H), 7.97-7.82 (m, 3H), 7.57-7.32 (m, 7H), 7.20-7.09 (m, 2H), 4.69-4.57 (m, 1H), 4.40 (s, 2H), 3.86-3.64 (m, 11H), 3.62-3.43 (m, 6H), 3.27-3.18 (m, 2H), 2.95-2.84 (m, 2H), 2.82-2.73 (m, 2H), 2.54-2.42 (m, 4H), 2.24 (m, 2H), 2.11-2.03 (m, 2H), 1.77 (m, 2H), 1.57 (m, 1H), 1.35-1.21 (m, 6H). ¹⁹F NMR (377 MHz, METHANOL-d₄) δ 120.7.

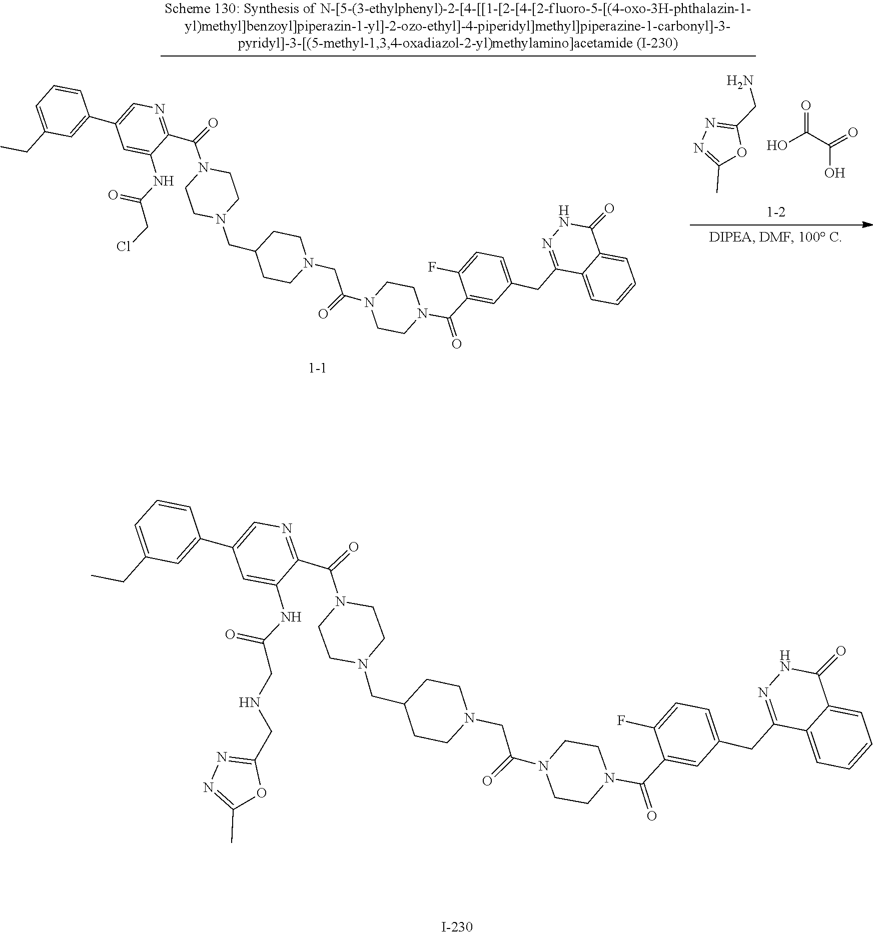

To a solution of Intermediate 1-1 (100 mg, 112.30 μmol, 1 eq) in DMF (2 mL) was added DIEA (72.57 mg, 561.52 μmol, 97.81 μL, 5 eq) and Intermediate 1-2 (34.22 mg, 168.46 μmol, 1.5 eq, oxalic acid salt). The mixture was stirred at 100° C. for 3 hrs. The mixture was filtered. The filtrate was purified by prep-HPLC (column: Waters Xbridge 150*25 mm*5 um; mobile phase: [water (NH₄HCO₃)-ACN]; gradient: 28%-58% B over 9 min). The eluent was concentrated under reduced pressure to remove ACN, then lyophilized to afford I-230 (14.39 mg, 14.63 μmol, 13.03% yield, 98.3% purity) as an off-white solid. LCMS (Method E): Rt=0.419 min, [M+H]⁺=967.4. ¹H NMR (400 MHz, METHANOL-d₄) δ=8.71 (d, J=2.0 Hz, 1H), 8.59 (d, J=2.0 Hz, 1H), 8.35 (m, 1H), 7.95-7.77 (m, 3H), 7.57-7.45 (m, 3H), 7.43-7.34 (m, 2H), 7.30 (d, J=7.6 Hz, 1H), 7.15 (m, 1H), 4.37 (s, 2H), 4.08 (s, 2H), 3.84-3.64 (m, 6H), 3.58-3.47 (m, 6H), 3.29-3.14 (m, 3H), 2.88 (m, 2H), 2.73 (m, 2H), 2.58-2.43 (m, 7H), 2.23 (m, 2H), 2.14-2.02 (m, 2H), 1.76 (m, 2H), 1.60-1.48 (m, 1H), 1.36-1.14 (m, 6H). ¹⁹FNMR (377 MHz, METHANOL-d₄) δ=−120.7.

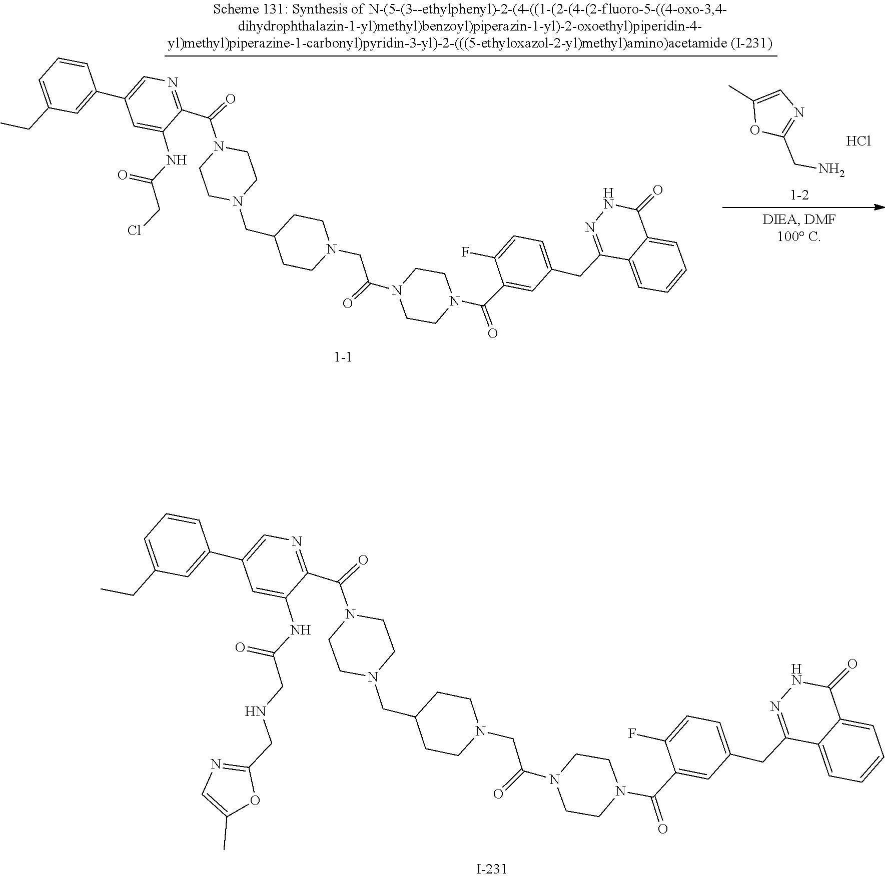

To a solution of Intermediate 1-1 (100 mg, 112.30 μmol, 1 eq) in DMF (2 mL) was added DIEA (72.57 mg, 561.52 μmol, 97.81 μL, 5 eq) and Intermediate 1-2 (25.03 mg, 168.46 μmol, 1.5 eq, HCl salt). The mixture was stirred at 100° C. for 3 hrs. The mixture was filtered, the filtrate was purified by prep-HPLC (column: Phenomenex luna C18 150*25 mm*10 um; mobile phase: [water (FA)-ACN]; gradient: 5%-35% B over 9 min), the eluent was concentrated under reduced pressure to remove ACN, then lyophilized to afford I-231 (22.97 mg, 22.69 μmol, 20.21% yield, formic acid salt) as an off-white solid. LCMS (Method E): Rt=0.417 min, [M+H]⁺=966.4. ¹H NMR (400 MHz, METHANOL-d₄) δ=8.62 (m, 2H), 8.36 (m, 1H), 8.29 (s, 2H), 7.98-7.91 (m, 1H), 7.89-7.79 (m, 2H), 7.51 (m, 2H), 7.44-7.27 (m, 3H), 7.16 (m, 1H), 6.72 (d, J=0.8 Hz, 1H), 4.37 (s, 2H), 4.19-4.07 (m, 2H), 3.95 (s, 2H), 3.84-3.68 (m, 5H), 3.59-3.48 (m, 8H), 3.38 (br s, 3H), 3.03-2.93 (m, 2H), 2.76-2.69 (m, 2H), 2.68-2.54 (m, 4H), 2.39 (m, 2H), 2.29 (d, J=0.8 Hz, 3H), 2.03 (m, 2H), 1.93 (m, 1H), 1.60-1.47 (m, 2H), 1.28 (m, 3H). ¹⁹FNMR (377 MHz, METHANOL-d₄) δ=−120.7.

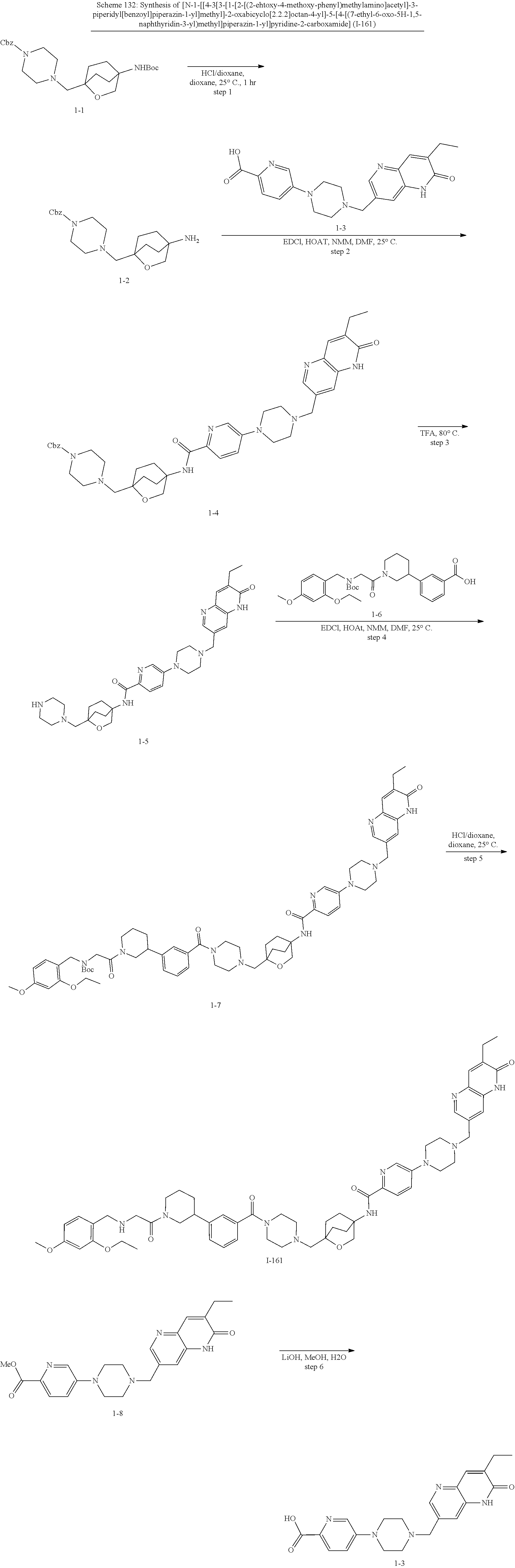

### Step 1: Synthesis of Intermediate 1-2

To a solution of intermediate 1-1 (2 g, 4.35 mmol, 1 eq) in dioxane (10 mL) was added HCl/dioxane (4 M, 10 mL, 9.19 eq). The mixture was stirred at 25° C. for 0.5 hr. The reaction mixture was concentrated under reduced pressure to give intermediate 1-2 (2.3 g, crude, HCl salt) as a white solid. LCMS (Method E): Retention time: 0.337 min, [M+H]⁺=360.1.

### Step 2: Synthesis of Intermediate 1-4

To a solution of Intermediate 1-3 (1 g, 2.54 mmol, 1 eq) in DMF (10 mL) was added EDCI (1.46 g, 7.63 mmol, 3 eq), HOAt (1.04 g, 7.63 mmol, 3 eq), NMM (1.29 g, 12.71 mmol, 1.40 mL, 5 eq) and intermediate 1-2 (1.37 g, 3.46 mmol, 1.36 eq, HCl salt). The mixture was stirred at 25° C. for 1 hr. The reaction mixture was diluted into water (40 mL) and extracted with ethyl acetate (15 mL×3). The combined organic layers were washed with brine (10 mL×2), dried over anhydrous sodium sulfate, filtered and concentrated under reduced pressure to give intermediate 1-4 (2 g, crude) as a yellow solid. LCMS (Method G): Retention time: 0.658 min, [M+H]⁺=735.5.

### Step 3: Synthesis of Intermediate 1-5

A solution of intermediate 1-4 (50 mg, 68.04 μmol, 1 eq) in TFA (1 mL) was stirred at 80° C. for 12 hr. The mixture was adjusted to pH 9 by slowly adding a saturated NaHCO₃ solution, and then extracted with chloroform/isopropyl alcohol 15 mL (5 mL×3). The combined organic layers were washed with brine (5 mL×2), dried over anhydrous sodium sulfate, filtered and concentrated under reduced pressure to give intermediate 1-5 (50 mg, crude) as a white solid. LCMS (Method E): Retention time: 0.334 min, [M+H]⁺=601.4.

### Step 4: Synthesis of Intermediate 1-7

To a solution of Intermediate 1-6 (29 mg, 55.49 μmol, 1 eq) in DMF (1 mL) was added EDCI (32 mg, 166.46 μmol, 3 eq), HOAt (23 mg, 166.46 μmol, 3 eq), NMM (28 mg, 277.43 μmol, 30.50 μL, 5 eq) and intermediate 1-5 (50 mg, 83.23 μmol, 1.5 eq). The mixture was stirred at 25° C. for 12 hr. The reaction mixture was diluted into water (10 mL) and extracted with ethyl acetate (5 mL×3). The combined organic layers were washed with brine (5 mL×2), dried over anhydrous sodium sulfate, filtered and concentrated under reduced pressure to give intermediate 1-7 (50 mg, crude) as yellow oil. LCMS (Method E): Retention time: 0.486 min, [M+H]⁺=1109.7.

### Step 5: Synthesis of I-161

To a solution of intermediate 1-7 (50 mg, 45.07 μmol, 1 eq) in dioxane (0.5 mL) was added HCl/dioxane (4 M, 0.5 mL, 44.37 eq). The mixture was stirred at 25° C. for 1 hr. The reaction mixture was concentrated under reduced pressure to give the crude product. The residue was dissolved in DMF (2 mL) and purified by prep-HPLC (column: Phenomenex luna C18 150*25 mm*10 um; mobile phase: [water(FA)-ACN]; gradient: 8%-38% B over 9 min) to give I-161 (10.92 mg, 10.60 μmol, 23.53% yield, 98% purity) as an off-white solid. LCMS (Method E): Retention time: 0.420 min, [M+H]⁺=1009.7. SFC: Retention time: 10.234 min. ¹H NMR (400 MHz, METHANOL-d4) δ=8.49 (d, J=1.6 Hz, 1H), 8.43 (s, 1H), 8.23 (d, J=2.8 Hz, 1H), 7.87-7.81 (m, 2H), 7.76 (s, 1H), 7.49-7.39 (m, 2H), 7.38-7.26 (m, 4H), 6.64-6.54 (m, 2H), 4.64-4.46 (m, 1H), 4.22-4.12 (m, 4H), 4.08 (s, 2H), 4.05-3.88 (m, 2H), 3.85-3.67 (m, 8H), 3.50-3.35 (m, 6H), 3.25-3.09 (m, 1H), 2.91-2.74 (m, 2H), 2.72-2.63 (m, 8H), 2.58-2.48 (m, 2H), 2.38-2.22 (m, 4H), 2.16-1.95 (m, 5H), 1.93-1.71 (m, 4H), 1.67-1.55 (m, 1H), 1.46 (d, J=8.0, 17.5 Hz, 3H), 1.28 (t, J=7.2 Hz, 3H).

### Step 6: Synthesis of Intermediate 1-3

To a solution of Intermediate 1-8 (10 g, 24.54 mmol, 1 eq.) in MeOH (50 mL) was added LiOH·H₂O (2.06 g, 49.08 mmol, 2.0 eq) and H₂O (50 mL) respectively. The resulting mixture was then allowed to reach 25° C. and stirred for 16 hrs. The resulting mixture was concentrated to remove MeOH (50 mL), and then the pH of residue was adjusted to 7. The solid was filtered off, collected and washed by water (500 mL) to afford the target product as an off-white solid. MeOH (10 ml) was added and it was recrystallized to afford the target product Intermediate 1-3 (9.0 g, 21.62 mmol, 88.08% yield, 94.5% purity) as an off white solid. LCMS (Method E): Rt=0.314 min, [M+H]⁺=394.1. ¹H NMR (400 MHz, DMSO-d6) δ=12.16-11.68 (m, 1H), 8.48-8.19 (m, 2H), 7.90-7.78 (m, 1H), 7.77-7.54 (m, 2H), 7.40-7.27 (m, 1H), 3.65-3.59 (m, 2H), 3.37-3.27 (m, 4H), 2.61-2.51 (m, 6H), 1.24-1.08 (m, 3H).

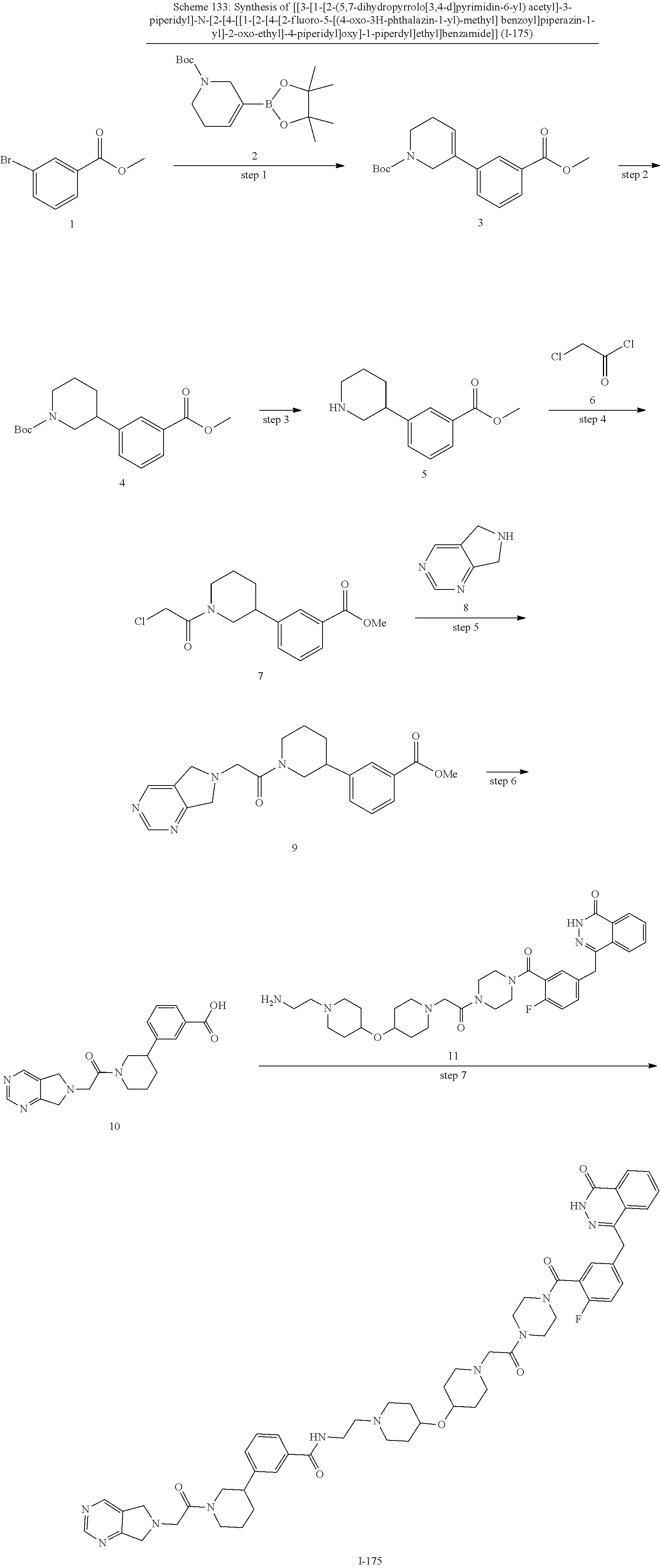

### Step 1: Synthesis of Intermediate 3

To a solution of Intermediate 1 (3 g, 13.95 mmol, 1 eq) and Intermediate 2 (4.31 g, 13.95 mmol, 1 eq) and K₂CO₃ (3.86 g, 27.90 mmol, 2 eq) in dioxane (30 mL) and H₂O (6 mL) was added Pd(dppf) Cl₂·CH₂Cl₂ (569.63 mg, 697.53 μmol, 0.05 eq) under N₂. The mixture was stirred at 100° C. for 12 hr. The aqueous phase was extracted with ethyl acetate (500 mL). The combined organic phase was washed with brine (500 mL), dried with Na₂SO₄, filtered and concentrated in vacuum. The residue was purified by column chromatography (SiO₂, Petroleum ether/Ethyl acetate=5/1 to 3/1). Intermediate 3 (4 g, 12.23 mmol, 87.63% yield, 97% purity) was obtained as a yellow oil. LCMS (Method E): Retention time: 0.669 min, [M+H]⁺=318.2. ¹H NMR (400 MHz, CHLOROFORM-d) δ=8.06 (s, 1H), 7.94 (d, J=7.6 Hz, 1H), 7.56 (br d, J=7.6 Hz, 1H), 7.44-7.38 (m, 1H), 6.28 (tt, J=1.6, 4.0 Hz, 1H), 4.30 (br s, 2H), 3.93 (s, 3H), 3.56 (br t, J=5.6 Hz, 2H), 2.34 (br s, 2H), 1.51 (s, 9H).

### Step 2: Synthesis of Intermediate 4

To a solution of Intermediate 3 (4 g, 12.60 mmol, 1 eq) in MeOH (40 mL) was added Pd/C (2 g, 1.88 mmol, 10% purity, 1.49e−1 eq) under H₂ (50 psi). The mixture was stirred at 60° C. for 12 hr under H₂ (50 psi). The mixture was filtered and concentrated under reduced pressure. Intermediate 4 (4 g, crude) was obtained as yellow oil. LCMS (Method E): Retention time: 0.929 min, [M−56+H]⁺=264.1.

### Step 3: Synthesis of Intermediate 5

To a solution of Intermediate 4 (2 g, 6.26 mmol, 1 eq) in dioxane (20 mL) was added HCl/dioxane (4 M, 10 mL, 6.39 eq). The mixture was stirred at 25° C. for 1 h. The mixture was filtered and concentrated under reduced pressure to give Intermediate 5 (2 g, crude) as a yellow solid. It was used in the next step directly without further purification. LCMS (Method E): Retention time: 0.446 min, [M+H]⁺=220.2.

### Step 4: Synthesis of Intermediate 7

To a solution of Intermediate 5 (2 g, 9.12 mmol, 1 eq) in DCM (20 mL) was added DIEA (3.54 g, 27.36 mmol, 4.77 mL, 3 eq) and Intermediate 6 (1.55 g, 13.68 mmol, 1.09 mL, 1.5 eq) at 0° C. The mixture was stirred at 25° C. for 2 h. The aqueous phase was extracted with ethyl acetate (100 mL). The combined organic phase was washed with brine (100 mL), dried with Na₂SO₄, filtered and concentrated in vacuum. The residue was purified by column chromatography (SiO₂, Petroleum ether/Ethyl acetate=3/1 to 1/1). Intermediate 7 (1.7 g, 5.63 mmol, 61.76% yield, 98% purity) was obtained as a yellow solid. LCMS (Method E): Retention time: 0.571 min, [M+H]⁺=296.1. ¹H NMR (400 MHz, CHLOROFORM-d) δ=7.98-7.89 (m, 2H), 7.49-7.36 (m, 2H), 4.75-4.63 (m, 1H), 4.18-4.02 (m, 2H), 3.93 (d, J=5.3 Hz, 3H), 3.23-3.09 (m, 1H), 2.95-2.59 (m, 2H), 2.11 (br d, J=11.0 Hz, 1H), 1.99-1.58 (m, 4H).

### Step 5: Synthesis of Intermediate 9

To a solution of Intermediate 7 (900 mg, 3.04 mmol, 1 eq) and Intermediate 8 (442.35 mg, 3.65 mmol, 1.2 eq) in ACN (8 mL) was added K₂CO₃ (1.26 g, 9.13 mmol, 3 eq) and NaI (45.61 mg, 304.30 μmol, 0.1 eq). The mixture was stirred at 80° C. for 1 hr. The aqueous phase was extracted with ethyl acetate (300 mL). The combined organic phase was washed with brine (300 mL), dried with Na₂SO₄, filtered and concentrated in vacuum. The residue was purified by prep-HPLC (column: Waters Xbridge BEH C18 250*50 mm*10 um; mobile phase: [water (NH₄HCO₃)-ACN]; gradient: 15%-45% B over 20 min). Intermediate 9 (500 mg, 1.30 mmol, 42.76% yield, 99% purity) was obtained as a yellow solid. LCMS (Method E): Retention time: 0.434 min, [M+H]⁺=381.2.

### Step 6: Synthesis of Intermediate 10

To a solution of Intermediate 9 (220 mg, 578.28 μmol, 1 eq) in MeOH (2 mL), THF (2 mL), and H₂O (2 mL) was added LiOH·H₂O (48.53 mg, 1.16 mmol, 2 eq). The mixture was stirred at 25° C. for 12 h. The mixture was filtered and concentrated under reduced pressure to give Intermediate 10 (200 mg, crude) as a yellow solid. It was used in the next step directly without further purification. LCMS (Method E): Retention time: 0.445 min, [M+H]⁺=367.2.

### Step 7: Synthesis of I-175

To a solution of Intermediate 10 (100 mg, 272.92 μmol, 1 eq) and Intermediate 11 (237.79 mg, 354.79 μmol, 1.3 eq, HCl salt) in DMF (3 mL) was added EDCI (104.64 mg, 545.83 μmol, 2 eq), NMM (82.81 mg, 818.75 μmol, 90.02 μL, 3 eq) and HOBt (36.88 mg, 272.92 μmol, 1 eq). The mixture was stirred at 25° C. for 1 h. The aqueous phase was extracted with ethyl acetate (100 mL). The combined organic phase was washed with brine (100 mL), dried with Na₂SO₄, filtered and concentrated in vacuum. The residue was purified by prep-HPLC (column: Waters Xbridge C18 150*50 mm*10 um; mobile phase: [water (NH₄HCO₃)-ACN]; gradient: 17%-47% B over 10 min). I-175 (48.22 mg, 49.10 μmol, 17.99% yield, 100% purity) was obtained as a white solid. LCMS (Method E): Retention time: 0.428 min, [M+H]⁺=982.4. SFC: Retention time: 1.490 min, 2.231 min. ¹H NMR (400 MHz, DMSO-d₆) δ=12.74-12.44 (m, 1H), 8.99 (s, 1H), 8.66 (s, 1H), 8.42-8.31 (m, 1H), 8.26 (d, J=7.6 Hz, 1H), 8.01-7.93 (m, 1H), 7.92-7.79 (m, 2H), 7.76-7.63 (m, 2H), 7.48-7.32 (m, 4H), 7.23 (t, J=8.8 Hz, 1H), 4.53-4.39 (m, 1H), 4.33 (s, 2H), 4.12-4.03 (m, 3H), 3.94 (brt, J=14.8 Hz, 1H), 3.82-3.43 (m, 7H), 3.35 (s, 6H), 3.21-2.99 (m, 5H), 2.81-2.55 (m, 6H), 2.43 (brt, J=6.4 Hz, 2H), 2.19-2.02 (m, 4H), 1.92 (br d, J=10.4 Hz, 1H), 1.74 (br s, 6H), 1.60-1.26 (m, 5H). ¹⁹F NMR (377 MHz, DMSO-d₆) δ=−119.76.

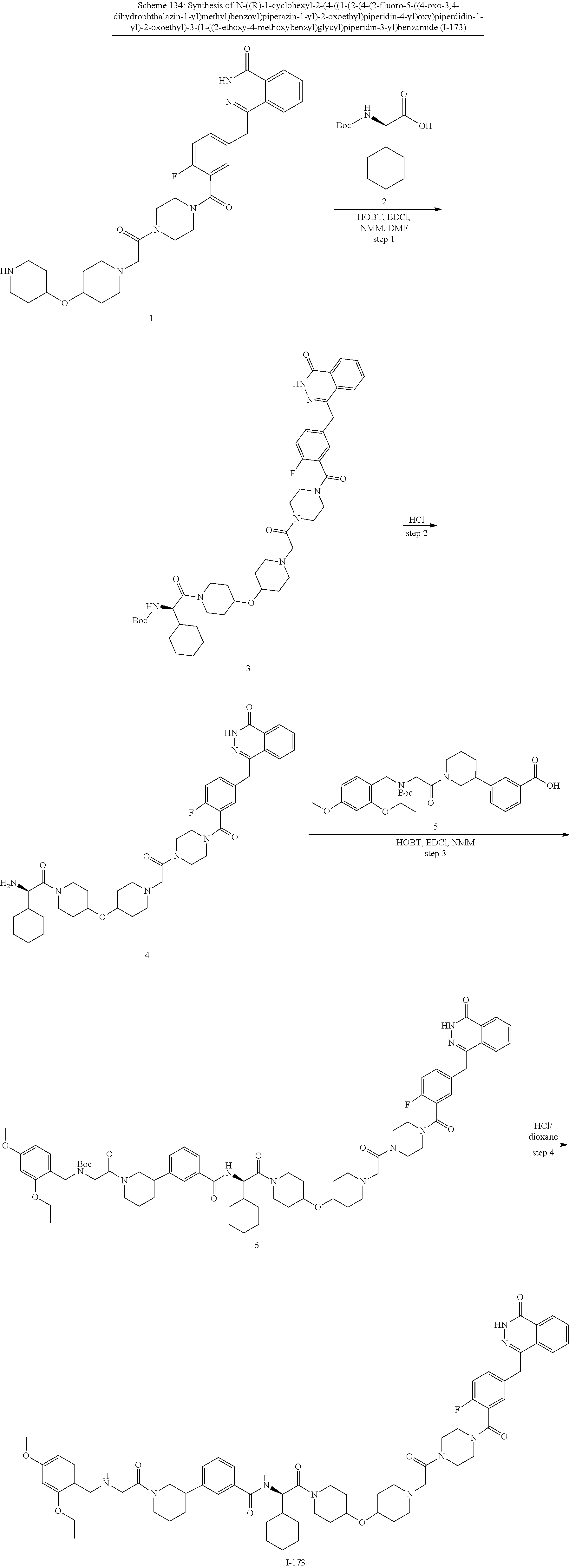

### Step 1: Synthesis of Intermediate 3

To a solution of Intermediate 1 (700 mg, 1.19 mmol, 1 eq) and Intermediate 2 (365.94 mg, 1.42 mmol, 1.2 eq) in DMF (10 mL) was added EDCI (454.35 mg, 2.37 mmol, 2 eq), HOBt (320.26 mg, 2.37 mmol, 2 eq) and NMM (359.60 mg, 3.56 mmol, 390.87 μL, 3 eq), the reaction mixture was stirred at 25° C. for 1 hr. The reaction mixture was diluted with H₂O (100 mL) and extracted with EA (100 mL*3). The combined organic layers were dried over Na₂SO₄, filtered and concentrated under reduced pressure to give a residue. The residue was purified by flash silica gel chromatography (ISCO; 20 g SepaFlash Silica Flash Column, Eluent of 0˜10% MeOH/DCM @80 mL/min). Intermediate 3 (560 mg, 637.86 μmol, 53.83% yield, 94.54% purity) was obtained as yellow solid. LCMS (Method E): Retention time: 0.534 min, [M+H]⁺=830.5.

### Step 2: Synthesis of Intermediate 4

To a solution of Intermediate 3 (200 mg, 240.96 μmol, 1 eq) in dioxane (1 mL) and MeOH (1 mL) was added HCl/dioxane (4 M, 2.00 mL, 33.20 eq) and the mixture was stirred at 25° C. for 1 hr. The reaction mixture was concentrated under reduced pressure to give Intermediate 4 (180 mg, crude) as a yellow solid. It was used in the next step without further purification. LCMS (Method E): Retention time: 0.441 min, [M+H]⁺=730.4.

### Step 3: Synthesis of Intermediate 6

To a solution of Intermediate 5 (155.85 mg, 295.94 μmol, 1.2 eq) and Intermediate 4 (180 mg, 246.62 μmol, 1 eq) in DMF (3 mL) was added EDCI (94.55 mg, 493.23 μmol, 2 eq), HOBt (66.65 mg, 493.23 μmol, 2 eq) and NMM (74.83 mg, 739.85 μmol, 81.34 μL, 3 eq), and the reaction mixture was stirred at 25° C. for 1 hr. The reaction mixture was diluted with H₂O (20 mL) and extracted with EA (20 mL*3). The combined organic layers were dried over Na₂SO₄, filtered and concentrated under reduced pressure to give Intermediate 6 (300 mg, crude) as yellow oil. It was used in the next step without further purification. LCMS (Method E): Retention time: 0.601 min, [M+H]⁺=1238.8.

### Step 4: Synthesis of I-173

To a solution of Intermediate 6 (200 mg, 161.49 μmol, 1 eq) in dioxane (2 mL) was added HCl/dioxane (4 M, 2 mL), and the reaction mixture was stirred at 25° C. for 1 hr. The reaction mixture was adjusted to pH=7 by addition of TEA and concentrated under reduced pressure to give a residue. The residue was purified by prep-HPLC (column: Phenomenex luna C18 150*25 mm*10 um; mobile phase: [water (FA)-ACN]; gradient: 18%-48% B over 10 min). I-173 (44.09 mg, 36.58 μmol, 22.65% yield, 98.27% purity, FA salt) was obtained as a white solid. LCMS (Method E): Retention time: 0.512 min, [M+H]⁺=1138.4. SFC: Retention time: 3.428 min, 3.633 min. ¹H NMR (400 MHz, DMSO-d₆) δ=12.60 (s, 1H), 8.55-8.40 (m, 1H), 8.30 (s, 1H), 8.19 (s, 1H), 8.01-7.70 (m, 5H), 7.40 (brdd, J=8.0, 16.4 Hz, 4H), 7.28-7.15 (m, 2H), 6.58-6.44 (m, 2H), 4.82-4.72 (m, 1H), 4.51-4.27 (m, 4H), 4.02 (br d, J=7.6 Hz, 2H), 3.80 (br d, J=8.8 Hz, 4H), 3.74 (br d, J=4.8 Hz, 4H), 3.64 (br d, J=9.6 Hz, 4H), 3.53-3.36 (m, 6H), 3.18-2.99 (m, 6H), 2.76-2.57 (m, 4H), 2.22-2.05 (m, 2H), 1.93-1.56 (m, 13H), 1.48-0.95 (m, 13H). ¹⁹F NMR (377 MHz, DMSO-d₆) δ=−119.77.

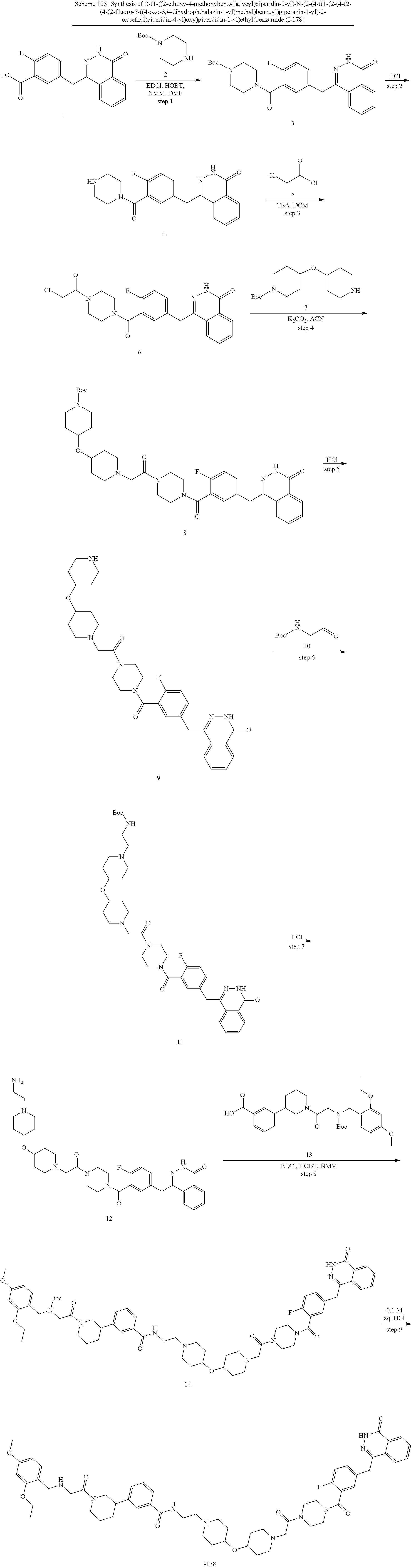

### Step 1: Synthesis of Intermediate 3

To a solution of Intermediate 1 (3 g, 10.06 mmol, 1 eq) and Intermediate 2 (2.25 g, 12.07 mmol, 1.2 eq) in DMF (30 mL) was added EDCI (3.86 g, 20.12 mmol, 2 eq), HOBt (2.72 g, 20.12 mmol, 2 eq) and NMM (3.05 g, 30.17 mmol, 3.32 mL, 3 eq), and the reaction mixture was stirred at 25° C. for 2 hr. The reaction mixture was diluted with H₂O (200 mL) and extracted with EA (200 mL*3). The combined organic layers were dried over Na₂SO₄, filtered and concentrated under reduced pressure to give a residue. The residue was purified by flash silica gel chromatography (ISCO; 40 g SepaFlash Silica Flash Column, Eluent of 0˜10% MeOH/DCM (0.1% NH₃. H₂O) @80 mL/min). Intermediate 3 (4.5 g, 9.62 mmol, 95.60% yield, 99.68% purity) was obtained as a yellow solid. LCMS (Method E): Retention time: 0.539 min, [M−56+H]⁺=411.1.

### Step 2: Synthesis of Intermediate 4

To a solution of Intermediate 3 (2.5 g, 5.36 mmol, 1 eq) in dioxane (10 mL) was added HCl/dioxane (4 M, 10 mL, 7.46 eq) and the reaction mixture was stirred at 25° C. for 1 hr. The reaction mixture was concentrated under reduced pressure to give Intermediate 4 (2 g, crude) as a yellow solid. It was used in the next step without further purification. LCMS (Method E): Retention time: 0.382 min, [M+H]⁺=367.1.

### Step 3: Synthesis of Intermediate 6

To a solution of Intermediate 4 (2 g, 5.46 mmol, 1 eq) in DCM (20 mL) was added TEA (2.76 g, 27.29 mmol, 3.80 mL, 5 eq). Then Intermediate 5 (924.78 mg, 8.19 mmol, 652.17 μL, 1.5 eq) was dropwise added at 0° C. and the reaction mixture was stirred at 25° C. for 1 hr. The reaction mixture was diluted with H₂O (100 mL) and extracted with DCM (100 mL*3). The combined organic layers were dried over Na₂SO₄, filtered and concentrated under reduced pressure to give a residue. The residue was purified by flash silica gel chromatography (ISCO; 40 g SepaFlash Silica Flash Column, Eluent of 0˜10% MeOH/DCM @100 mL/min). Intermediate 6 (2.2 g, 4.76 mmol, 87.16% yield, 95.78% purity) was obtained as a yellow solid. LCMS (Method E): Retention time: 0.470 min, [M+H]⁺=443.1.

### Step 4: Synthesis of Intermediate 8

To a solution of Intermediate 6 (1 g, 2.26 mmol, 1 eq) and Intermediate 7 (770.59 mg, 2.71 mmol, 1.2 eq) in ACN (10 mL) was added K₂CO₃ (936.20 mg, 6.77 mmol, 3 eq) and NaI (33.85 mg, 225.80 μmol, 0.1 eq) and the reaction mixture was stirred at 100° C. for 1 hr. The reaction mixture was filtered and concentrated under reduced pressure to give a residue. The crude product was triturated with DCM/MTBE and then filtered, and the filter cake was dried under vacuum to give Intermediate 8 (1 g, 1.45 mmol, 64.11% yield) as a brown solid. LCMS (Method E): Retention time: 0.478 min, [M+H]⁺=691.3.

### Step 5: Synthesis of Intermediate 9

To a solution of Intermediate 8 (1 g, 1.45 mmol, 1 eq) in dioxane (4 mL) and MeOH (1 mL) was added HCl/dioxane (4 M, 5 mL, 13.82 eq) and the reaction mixture was stirred at 25° C. for 1 hr. The reaction mixture was concentrated under reduced pressure to give Intermediate 9 (860 mg, crude) as a yellow solid. It was used in the next step without further purification. LCMS (Method E): Retention time: 0.391 min, [M+H]⁺=591.3.

### Step 6: Synthesis of Intermediate 11

To a solution of Intermediate 9 (760 mg, 1.29 mmol, 1 eq) and Intermediate 10 (245.77 mg, 1.54 mmol, 1.2 eq) in MeOH (8 mL) was added AcOH (7.73 mg, 128.66 μmol, 7.37 μL, 0.1 eq) and the mixture was stirred at 25° C. for 1 hr, then NaBH₃CN (161.71 mg, 2.57 mmol, 2 eq) was added, the reaction mixture was stirred at 25° C. for 4 hr. Then NaBH₃CN (161.71 mg, 2.57 mmol, 2 eq) was added and the reaction mixture was stirred at 40° C. for 12 hr. The reaction mixture was concentrated under reduced pressure to give a residue. The residue was purified by flash silica gel chromatography (ISCO; 20 g SepaFlash Silica Flash Column, Eluent of 2-20% MeOH/DCM (0.1% NH₃·H₂O) @70 mL/min). Intermediate 11 (450 mg, 601.11 μmol, 46.72% yield, 98.03% purity) was obtained as a white solid. LCMS (Method E): Retention time: 0.478 min, [M+H]⁺=734.6.

### Step 7: Synthesis of Intermediate 12

To a solution of Intermediate 11 (300 mg, 408.79 μmol, 1 eq) in dioxane (2 mL) was added HCl/dioxane (4 M, 2 mL, 19.57 eq), and the reaction mixture was stirred at 25° C. for 1 hr. The reaction mixture was concentrated under reduced pressure to give Intermediate 12 (260 mg, crude) as a white solid. It was used into next step without further purification. LCMS (Method E): Retention time: 0.376 min, [M+H]⁺=634.4.

### Step 8: Synthesis of Intermediate 14

To a solution of Intermediate 12 (200 mg, 315.58 μmol, 1 eq) and Intermediate 13 (199.43 mg, 378.70 μmol, 1.2 eq) in DMF (3 mL) was added EDCI (120.99 mg, 631.16 μmol, 2 eq), HOBt (85.28 mg, 631.16 μmol, 2 eq) and NMM (95.76 mg, 946.74 μmol, 104.09 μL, 3 eq), and the reaction mixture was stirred at 25° C. for 2 hr. The reaction mixture was diluted with H₂O (30 mL) and extracted with EA (30 mL*3). The combined organic layers were dried over Na₂SO₄, filtered and concentrated under reduced pressure to give a residue. The residue was purified by prep-HPLC (column: Phenomenex luna C18 150*25 mm*10 um; mobile phase: [water (FA)-ACN]; gradient: 19%-49% B over 10 min). Intermediate 14 (150 mg, 128.21 μmol, 73.23% yield, 97.64% purity) was obtained as a white solid. LCMS (Method E): Retention time: 0.514 min, [M+H]⁺=1142.4. ¹H NMR (400 MHz, DMSO-d₆) δ=12.60 (s, 1H), 8.74-8.61 (m, 1H), 8.26 (d, J=7.5 Hz, 1H), 8.14 (s, 1H), 8.00-7.93 (m, 1H), 7.92-7.70 (m, 4H), 7.49-7.33 (m, 4H), 7.23 (t, J=9.0 Hz, 1H), 7.10-7.00 (m, 1H), 6.54-6.42 (m, 2H), 4.48-4.40 (m, 1H), 4.32 (s, 2H), 4.24 (s, 1H), 4.06-3.95 (m, 4H), 3.72 (br d, J=9.4 Hz, 3H), 3.57 (br s, 4H), 3.43 (br s, 4H), 3.26-3.14 (m, 4H), 3.08-3.02 (m, 2H), 2.86 (br d, J=2.0 Hz, 2H), 2.76-2.64 (m, 4H), 2.35-2.19 (m, 2H), 1.95-1.70 (m, 7H), 1.61 (br s, 2H), 1.51-1.41 (m, 2H), 1.36 (br d, J=4.9 Hz, 9H), 1.32-1.27 (m, 2H). ¹⁹F NMR (377 MHz, DMSO-d₆) δ=−119.76.

### Step 9: Synthesis of I-178

To a solution of Intermediate 14 (150 mg, 131.31 μmol, 1 eq) in dioxane (2 mL) was added HCl/dioxane (4 M, 2 mL, 60.93 eq) and the reaction mixture was stirred at 25° C. for 1 hr. The reaction mixture was concentrated under reduced pressure to give a residue. The residue was purified by prep-HPLC (column: Phenomenex luna C18 150*25 mm*10 um; mobile phase: [water (FA)-ACN]; gradient: 5%-35% B over 10 min). I-178 (64.72 mg, 59.47 μmol, 45.29% yield, 100% purity, FA salt) was obtained as a white solid. LCMS (Method E): Retention time: 0.455 min, [M+H]⁺=1042.4. SFC: Retention time: 2.625 min, 3.932 min. ¹H NMR (400 MHz, DMSO-d₆) δ=12.61 (s, 1H), 8.46 (br s, 1H), 8.27 (d, J=7.6 Hz, 1H), 8.21 (s, 3H), 8.00-7.94 (m, 1H), 7.92-7.81 (m, 2H), 7.79-7.66 (m, 2H), 7.48-7.35 (m, 4H), 7.27-7.19 (m, 2H), 6.58-6.46 (m, 2H), 4.44 (brt, J=12.0 Hz, 1H), 4.34 (s, 2H), 4.06-4.01 (m, 2H), 3.70-3.60 (m, 10H), 3.52-3.35 (m, 8H), 3.19-3.00 (m, 5H), 2.85-2.60 (m, 6H), 2.28-2.06 (m, 4H), 1.93 (br d, J=10.0 Hz, 1H), 1.78 (br d, J=9.2 Hz, 6H), 1.53-1.27 (m, 8H). ¹⁹F NMR (377 MHz, DMSO-d₆) δ=−119.77.

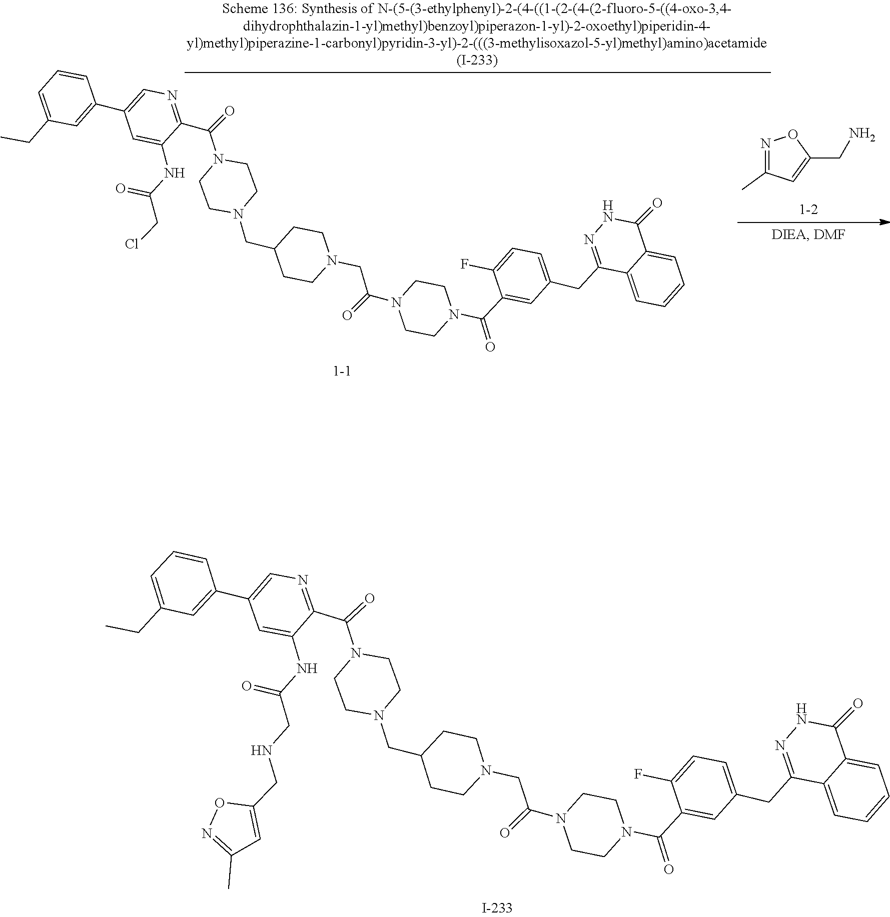

To a solution of Intermediate 1-1 (50 mg, 56.15 μmol, 1 eq) and Intermediate 1-2 (9.44 mg, 84.23 μmol, 1.5 eq) in DMF (1 mL) was added DIEA (36.29 mg, 280.76 μmol, 48.90 μL, 5 eq). The mixture was stirred at 40° C. for 2 hrs. The reaction mixture was washed with water and extracted with EA 3 times, and the separated organic phase was concentrated to give the crude product. The crude product was purified by prep-HPLC (column: YMC-Actus Triart C18 150*30 mm*7 um; mobile phase: [water (FA)-ACN]; gradient: 10%-40% B over 10 min) and the eluent was concentrated to remove MeCN and then lyophilized to afford I-233 (16.81 mg, 16.44 μmol, 29.28% yield, 99% purity, FA salt) as a white solid. LCMS (Method E): Rt=0.417 min, [M+H]⁺=966.7. HPLC: Rt=1.469 min. ¹H NMR (400 MHz, METHANOL-d₄) δ=8.73 (d, J=2.0 Hz, 1H), 8.61 (d, J=2.0 Hz, 1H), 8.48-8.42 (m, 1H), 8.39-8.35 (m, 1H), 7.98-7.93 (m, 1H), 7.90-7.80 (m, 2H), 7.55-7.48 (m, 3H), 7.43-7.35 (m, 1H), 7.40-7.34 (m, 1H), 7.31 (d, J=7.6 Hz, 1H), 7.17-7.00 (m, 1H), 6.33 (s, 1H), 4.38 (s, 2H), 3.96 (s, 2H), 3.92-3.66 (m, 8H), 3.65-3.50 (m, 5H), 3.46 (s, 3H), 3.38-3.33 (m, 2H), 2.80-2.64 (m, 4H), 2.60-2.48 (m, 4H), 2.31-2.28 (m, 2H), 2.26 (s, 3H), 1.95 (s, 2H), 1.78 (d, J=2.8 Hz, 1H), 1.52-1.37 (m, 2H), 1.29-1.15 (m, 3H). ¹⁹F NMR (377 MHz, METHANOL-d₄), δ=−120.750.

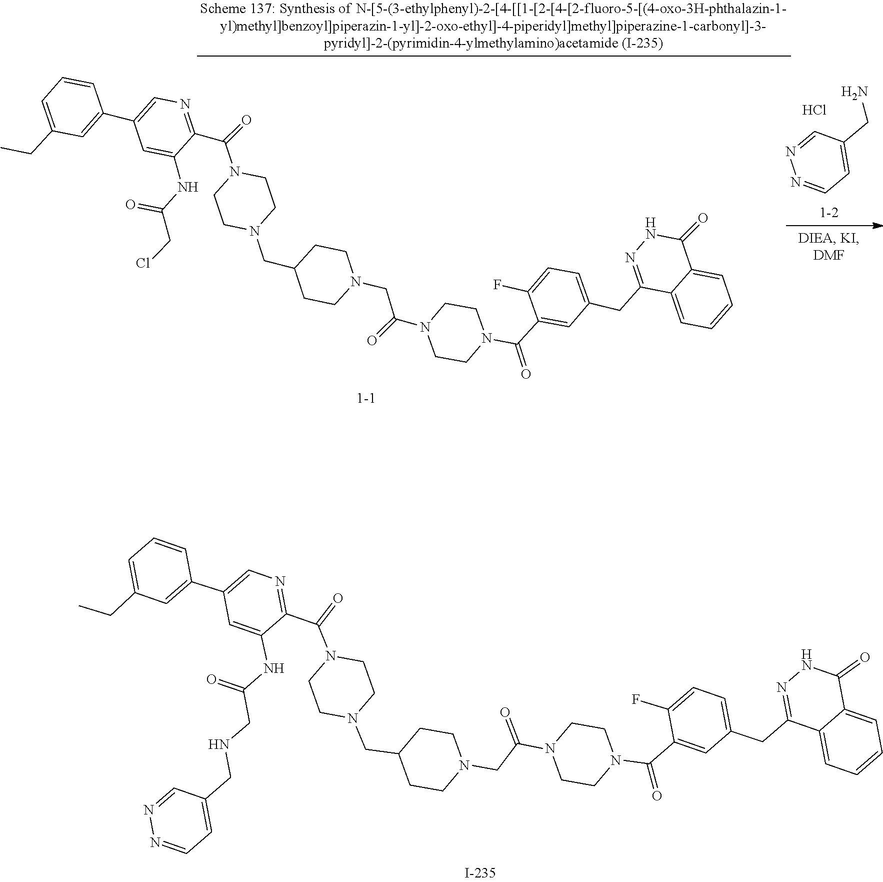 [SMILES: CCc1cccc(-c2cnc(C(=O)N3CCN(CC4CCN(CC(=O)N5CCN(C(=O)c6cc(Cc7n[nH]c(=O)c8ccccc78)ccc6F)CC5)CC4)CC3)c(NC(=O)CCl)c2)c1 ; NCc1ccnnc1 ; Cl ; CCc1cccc(-c2cnc(C(=O)N3CCN(CC4CCN(CC(=O)N5CCN(C(=O)c6cc(Cc7n[nH]c(=O)c8ccccc78)ccc6F)CC5)CC4)CC3)c(NC(=O)CNCc3ccnnc3)c2)c1]

To a solution of Intermediate 1-1 (50 mg, 56.15 μmol, 1 eq) and Intermediate 1-2 (12.26 mg, 84.23 μmol, 1.5 eq, HCl salt) in DMF (1 mL) was added DIEA (36.29 mg, 280.76 μmol, 48.90 μL, 5 eq) and KI (37.29 mg, 224.61 μmol, 4 eq). The mixture was stirred at 60° C. for 2 hrs. The reaction mixture was washed with water and extracted with EA 3 times, and the separated organic phase was concentrated to give the crude product. The crude product was purified by prep-HPLC (column: Phenomenex luna C18 150*25 mm*10 um; mobile phase: [water (FA)-ACN]; gradient: 30%-60% B over 15 min) and the eluent was concentrated to remove MeCN and then lyophilized to afford I-235 (31.45 mg, 31.17 μmol, 55.50% yield, 100% purity, FA salt) as an orange solid. LCMS (Method E): Rt=0.426 min, [M+H]⁺=963.8. ¹H NMR (400 MHz, DMSO-d₆) δ=12.60 (s, 1H), 9.35 (s, 1H), 9.20 (d, J=5.6 Hz, 1H), 8.83 (s, 1H), 8.64 (d, J=1.6 Hz, 1H), 8.26 (d, J=7.6 Hz, 1H), 8.13 (s, 1H), 7.99-7.94 (m, 1H), 7.92-7.76 (m, 3H), 7.56-7.51 (m, 2H), 7.46 (d, J=7.6 Hz, 2H), 7.38-7.31 (m, 2H), 7.26-7.20 (m, 1H), 6.53 (s, 1H), 4.34 (s, 3H), 3.86 (s, 2H), 3.74-3.57 (m, 6H), 3.53-3.41 (m, 7H), 3.20 (s, 1H), 3.00-2.80 (m, 2H), 2.70-2.65 (m, 3H), 2.45-2.32 (m, 3H), 2.15-2.10 (m, 2H), 1.97-1.69 (m, 4H), 1.51-1.35 (m, 2H), 1.30-1.15 (m, 5H). ¹⁹F NMR (377 MHz, DMSO-d₆), δ=−119.694.

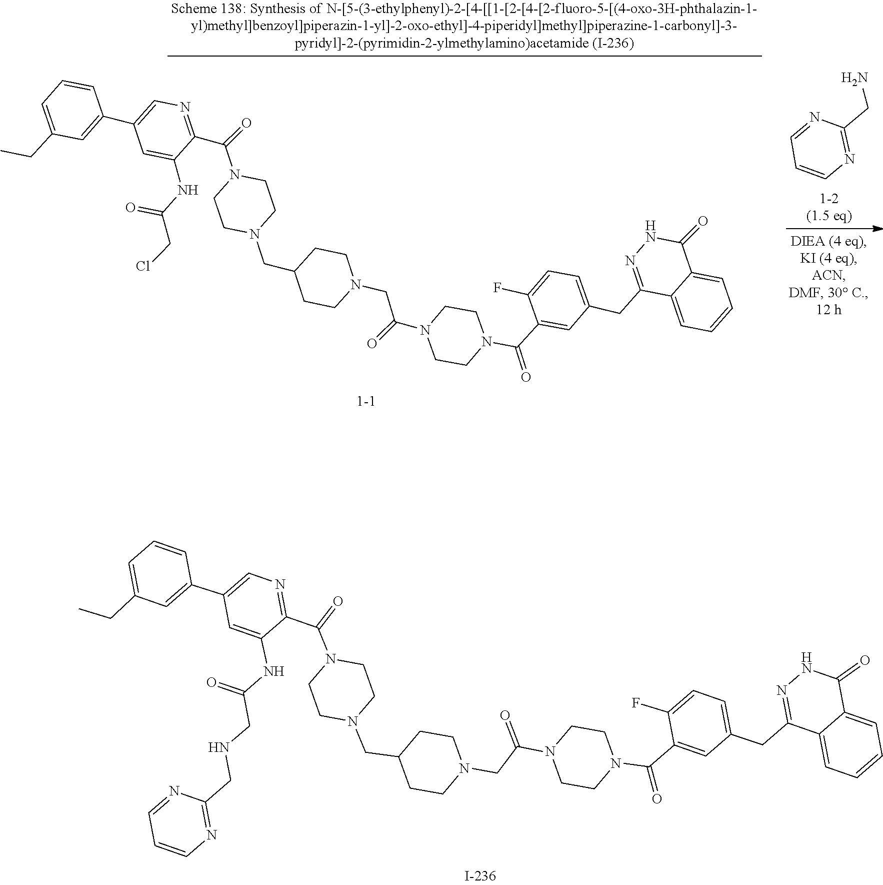 [SMILES: CCc1cccc(-c2cnc(C(=O)N3CCN(CC4CCN(CC(=O)N5CCN(C(=O)c6cc(Cc7n[nH]c(=O)c8ccccc78)ccc6F)CC5)CC4)CC3)c(NC(=O)CCl)c2)c1 ; NCc1ncccn1 ; CCc1cccc(-c2cnc(C(=O)N3CCN(CC4CCN(CC(=O)N5CCN(C(=O)c6cc(Cc7n[nH]c(=O)c8ccccc78)ccc6F)CC5)CC4)CC3)c(NC(=O)CNCc3ncccn3)c2)c1]

To a solution of Intermediate 1-1 (30 mg, 33.69 μmol, 1 eq) and Intermediate 1-2 (5.52 mg, 50.54 μmol, 1.5 eq) in ACN (0.5 mL) and DMF (0.1 mL) was added DIEA (17.42 mg, 134.76 μmol, 23.47 μL, 4 eq) and KI (22.37 mg, 134.76 μmol, 4 eq). Then the mixture was stirred at 30° C. for 12 hrs. The mixture was purified by prep-HPLC (column: YMC-Actus Triart C18 150*30 mm*7 um; mobile phase: [water (FA)-ACN]; gradient: 8%-38% B over 10 min) and then lyophilized to afford I-236 (18.76 mg, 19.48 μmol, 57.81% yield, 100% purity) was obtained as a white solid. LCMS (Method E): Rt=0.406 min, [M+H]⁺=963.5. ¹H NMR (400 MHz, METHANOL-d₄) δ=8.82 (d, J=4.8 Hz, 2H), 8.64 (d, J=1.6 Hz, 1H), 8.58 (s, 1H), 8.40-8.33 (m, 1H), 7.99-7.79 (m, 3H), 7.58-7.47 (m, 3H), 7.46-7.35 (m, 3H), 7.32 (d, J=7.2 Hz, 1H), 7.19-7.14 (m, 1H), 4.39 (s, 2H), 4.31-4.15 (m, 4H), 3.89-3.68 (m, 7H), 3.56 (br s, 6H), 3.45-3.33 (m, 3H), 3.18-2.99 (m, 2H), 2.78-2.71 (m, 2H), 2.68-2.51 (m, 4H), 2.38 (br d, J=7.2 Hz, 2H), 2.12-1.87 (m, 3H), 1.63-1.48 (m, 2H), 1.31-1.27 (m, 3H). ¹⁹FNMR (377 MHz, METHANOL-d₄), δ=−120.713.

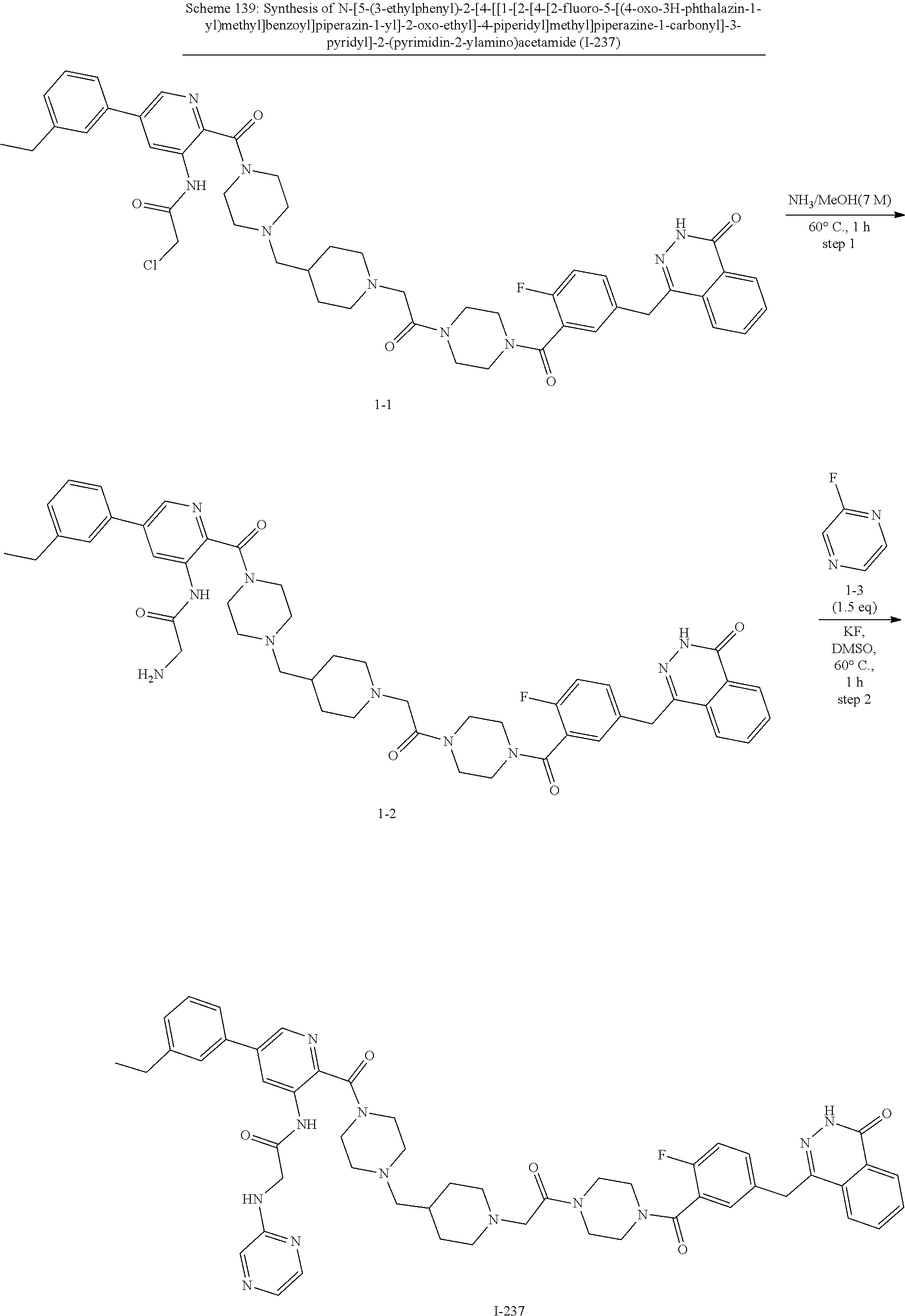 [SMILES: CCc1cccc(-c2cnc(C(=O)N3CCN(CC4CCN(CC(=O)N5CCN(C(=O)c6cc(Cc7n[nH]c(=O)c8ccccc78)ccc6F)CC5)CC4)CC3)c(NC(=O)CCl)c2)c1 ; CCc1cccc(-c2cnc(C(=O)N3CCN(CC4CCN(CC(=O)N5CCN(C(=O)c6cc(Cc7n[nH]c(=O)c8ccccc78)ccc6F)CC5)CC4)CC3)c(NC(=O)CN)c2)c1 ; Fc1cnccn1 ; CCc1cccc(-c2cnc(C(=O)N3CCN(CC4CCN(CC(=O)N5CCN(C(=O)c6cc(Cc7n[nH]c(=O)c8ccccc78)ccc6F)CC5)CC4)CC3)c(NC(=O)CNc3cnccn3)c2)c1 ; "I-237"]

### Step 1: Synthesis of Intermediate 1-2

A solution of Intermediate 1-1 (200 mg, 224.61 μmol, 1 eq) in NH₃/MeOH (5 mL) was stirred at 60° C. for 1 hr. The reaction mixture was concentrated to give a residue. The residue was purified by prep-HPLC (column: YMC-Actus Triart C18 150*30 mm*7 um; mobile phase: [water (FA)-ACN]; gradient: 8%-38% B over 10 min). The eluent was concentrated under reduced pressure to remove ACN, then lyophilized to afford Intermediate 1-2 (120 mg, 134.06 μmol, 59.69% yield, 97.308% purity) as a white solid. LCMS (Method E): Rt=0.409 min, [M+H]⁺=871.5.

### Step 2: Synthesis of I-237

To a solution of Intermediate 1-3 (9.91 mg, 101.03 μmol, 1.1 eq) and Intermediate 1-2 (80.00 mg, 91.85 μmol, 1 eq) in DMSO (0.5 mL) was added KF (16.01 mg, 275.54 μmol, 3 eq). The mixture was stirred at 60° C. for 1 hr. extracted with EA 3 times The reaction mixture was filtered to give a residue. The residue was purified by prep-HPLC (column: Waters Xbridge 150*25 mm*5 um; mobile phase: [water (NH₄HCO₃)-ACN]; gradient: 25%-55% B over 15 min). The eluent was concentrated under reduced pressure to remove ACN, then lyophilized to afford I-237 (25 mg, 26.34 μmol, 28.68% yield, 100% purity) as a white solid. LCMS (Method G): Rt=0.622 min, [M+H]⁺=949.6. ¹H NMR (400 MHz, METHANOL-d₄) δ=8.71 (s, 1H), 8.63-8.59 (m, 1H), 8.42-8.35 (m, 3H), 8.00-7.94 (m, 1H), 7.93-7.82 (m, 2H), 7.57-7.48 (m, 3H), 7.44 (t, J=7.6 Hz, 1H), 7.39 (d, J=4.4 Hz, 1H), 7.33 (d, J=7.6 Hz, 1H), 7.20-7.18 (m, 1H), 6.81-6.72 (m, 1H), 4.41 (s, 2H), 4.19 (s, 2H), 3.85-3.47 (m, 11H), 3.29-3.18 (m, 2H), 2.99-2.85 (m, 2H), 2.77-2.73 (m, 2H), 2.59-2.36 (m, 4H), 2.29-2.18 (m, 2H), 2.16-2.02 (m, 2H), 1.86-1.72 (m, 2H), 1.66-1.48 (m, 1H), 1.38-1.14 (m, 6H). ¹⁹F NMR (377 MHz, METHANOL-d₄), δ=−120.719.

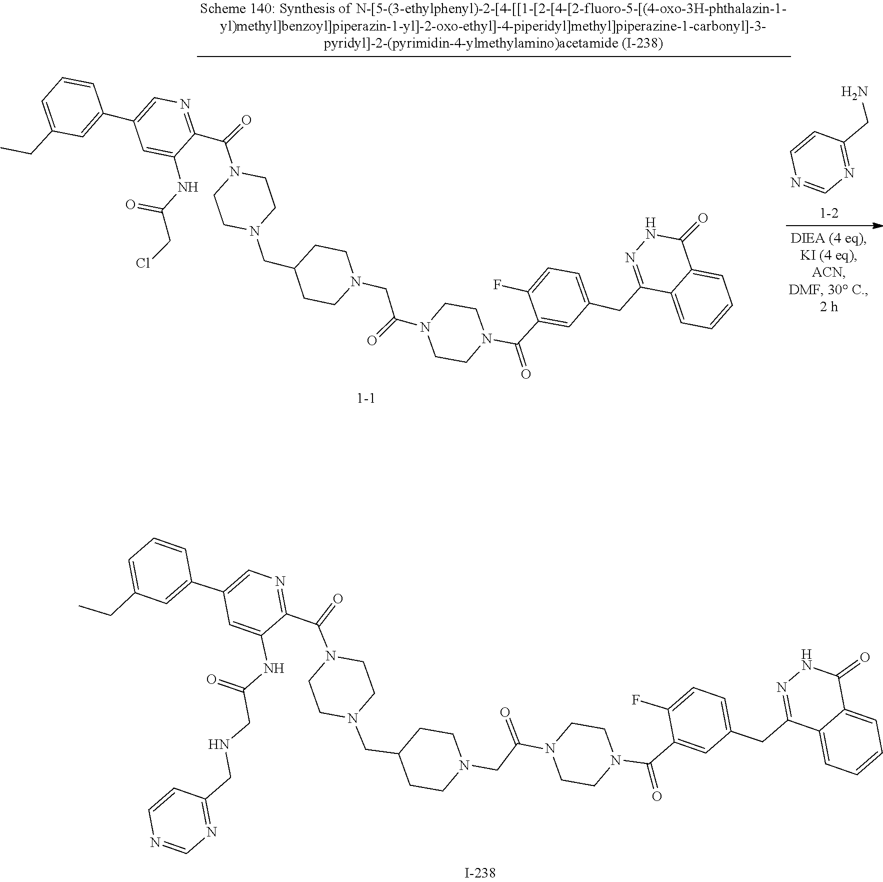 [SMILES: CCc1cccc(-c2cnc(C(=O)N3CCN(CC4CCN(CC(=O)N5CCN(C(=O)c6cc(Cc7n[nH]c(=O)c8ccccc78)ccc6F)CC5)CC4)CC3)c(NC(=O)CCl)c2)c1 ; NCc1ccncn1 ; CCc1cccc(-c2cnc(C(=O)N3CCN(CC4CCN(CC(=O)N5CCN(C(=O)c6cc(Cc7n[nH]c(=O)c8ccccc78)ccc6F)CC5)CC4)CC3)c(NC(=O)CNCc3ccncn3)c2)c1]

To a solution of Intermediate 1-1 (50 mg, 56.15 μmol, 1 eq) and Intermediate 1-2 (9.19 mg, 84.23 mol, 1.5 eq) in ACN (0.4 mL) and DMF (0.1 mL) was added DIEA (29.03 mg, 224.61 μmol, 39.12 μL, 4 eq) and KI (37.29 mg, 224.61 μmol, 4 eq). Then the mixture was stirred at 30° C. for 2 hrs. The mixture was diluted with MeOH (1 Ml) and purified by prep-HPLC (column: Phenomenex luna C18 150*25 mm*10 um; mobile phase: [water (FA)-ACN]; gradient: 1%-30% B over 1 min) and then lyophilized to afford I-238 (31.42 mg, 31.14 μmol, 55.45% yield, 100% purity, FA salt) as an off-white solid. LCMS (Method E): Rt=0.415 min, [M+H]⁺=963.5. ¹H NMR (400 MHz, METHANOL-d₄) δ=9.15 (s, 1H), 8.79 (d, J=5.2 Hz, 1H), 8.64 (s, 2H), 8.43-8.32 (m, 1H), 8.14 (br s, 1H), 8.02-7.93 (m, 1H), 7.93-7.80 (m, 2H), 7.75 (d, J=4.8 Hz, 1H), 7.59-7.47 (m, 3H), 7.47-7.30 (m, 3H), 7.20-7.14 (m, 1H), 4.39 (s, 2H), 4.35-4.22 (m, 2H), 4.17 (s, 2H), 3.91-3.76 (m, 4H), 3.72 (s, 3H), 3.58 (br d, J=16.4 Hz, 6H), 3.45-3.34 (m, 3H), 3.22-3.04 (m, 2H), 2.83-2.57 (m, 6H), 2.53-2.35 (m, 2H), 2.18-1.91 (m, 3H), 1.71-1.48 (m, 2H), 1.32-1.27 (m, 3H). ¹⁹FNMR (377 MHz, METHANOL-d₄), δ=−120.689.

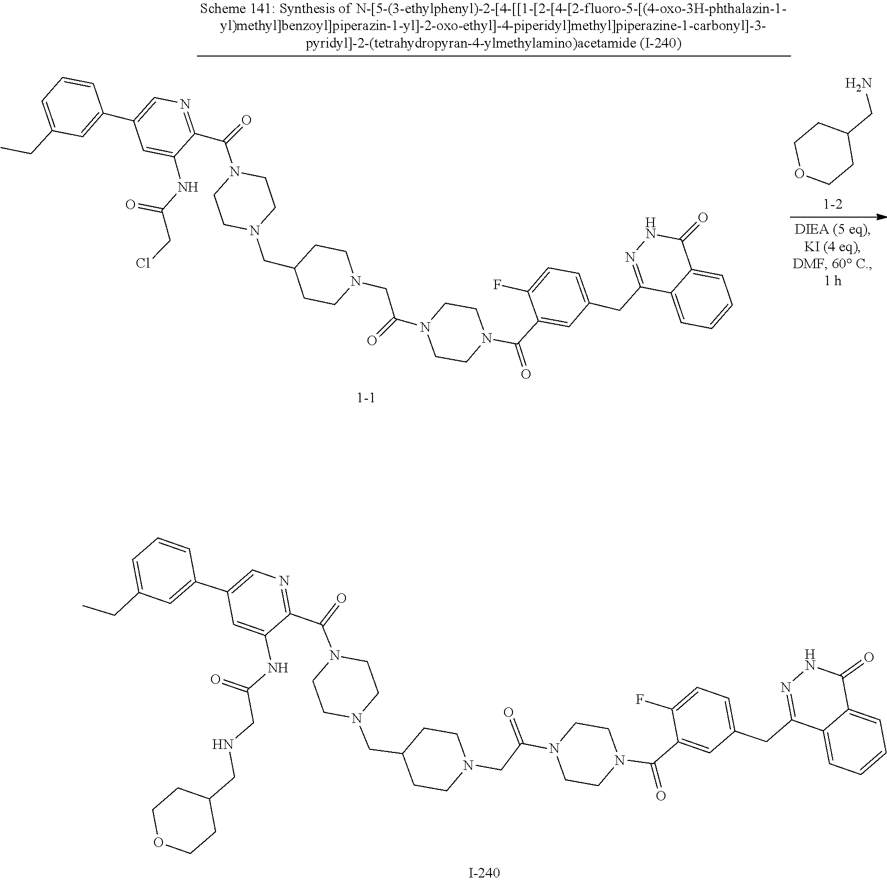 [SMILES: CCc1cccc(-c2cnc(C(=O)N3CCN(CC4CCN(CC(=O)N5CCN(C(=O)c6cc(Cc7n[nH]c(=O)c8ccccc78)ccc6F)CC5)CC4)CC3)c(NC(=O)CCl)c2)c1 ; NCC1CCOCC1 ; CCc1cccc(-c2cnc(C(=O)N3CCN(CC4CCN(CC(=O)N5CCN(C(=O)c6cc(Cc7n[nH]c(=O)c8ccccc78)ccc6F)CC5)CC4)CC3)c(NC(=O)CNCC3CCOCC3)c2)c1]

To a solution of Intermediate 1-1 (50 mg, 56.15 μmol, 1 eq) and Intermediate 1-2 (9.70 mg, 84.23 μmol, 1.5 eq) in DMF (0.5 mL) was added DIEA (36.29 mg, 280.76 μmol, 48.90 μL, 5 eq) and KI (37.29 mg, 224.61 μmol, 4 eq). The mixture was stirred at 60° C. for 1 hr. The reaction mixture was washed with water and extracted with EA 3 times, and the separated organic phase was concentrated to give the crude product. The crude product was purified by prep-HPLC (column: Phenomenex luna C18 150*25 mm*10 um; mobile phase: [water (FA)-ACN]; gradient: 2%-32% B over 15 min), then concentrated to remove organic solvents and lyophilized to give I-240 (14.02 mg, 14.47 μmol, 25.76% yield, 100% purity) as a white solid. LCMS (Method E): Rt=0.417 min, [M+H]⁺=969.5. ¹H NMR (400 MHz, METHANOL-d₄) δ=8.79 (d, J=2.0 Hz, 1H), 8.63-8.59 (m, 1H), 8.37 (d, J=7.6 Hz, 1H), 7.98-7.93 (m, 1H), 7.90-7.81 (m, 2H), 7.56-7.46 (m, 3H), 7.46-7.29 (m, 3H), 7.19-7.14 (m, 1H), 4.59 (s, 1H), 4.38 (s, 2H), 3.99-3.95 (m, 2H), 3.86-3.73 (m, 4H), 3.71-3.64 (m, 2H), 3.58-3.41 (m, 10H), 3.17-3.03 (m, 2H), 2.80-2.70 (m, 2H), 2.57 (d, J=6.4 Hz, 4H), 2.48 (s, 2H), 2.43-2.23 (m, 4H), 1.90-1.63 (m, 6H), 1.43-1.23 (m, 8H). ¹⁹F NMR (377 MHz, METHANOL-d₄), δ=−120.77.

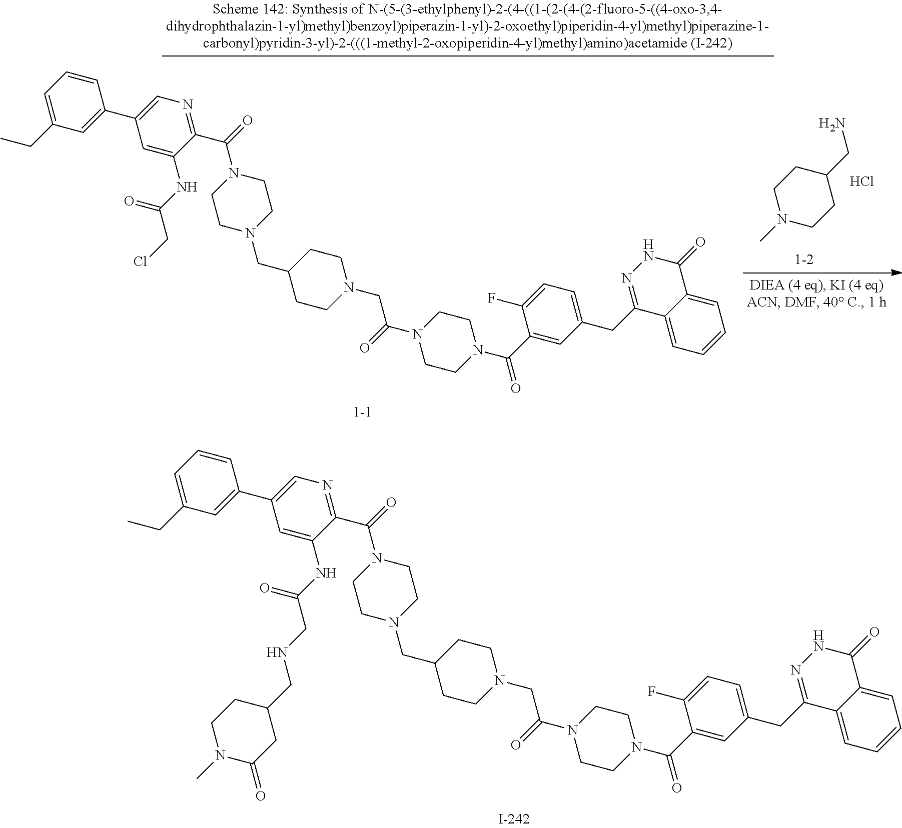

To a solution of Intermediate 1-1 (50 mg, 56.15 μmol, 1 eq) and Intermediate 1-2 (15.05 mg, 84.23 μmol, 1.5 eq) in ACN (1 mL) and DMF (0.5 mL) was added DIEA (29.03 mg, 224.61 μmol, 39.12 L, 4 eq) and KI (37.29 mg, 224.61 μmol, 4 eq). The mixture was stirred at 40° C. for 1 hr. The reaction mixture was filtered and the mother liquor was purified by prep-HPLC (column: Phenomenex luna C18 150*25 mm*10 um; mobile phase: [water (FA)-ACN]; gradient: 18%-48% B over 15 min). The eluent was concentrated under reduced pressure to remove ACN, then lyophilized to afford I-242 (25 mg, 25.10 μmol, 44.69% yield, 100% purity) as a white solid. LCMS (Method E): Rt=0.419 min, [M+H]⁺=996.6. SFC: Rt=2.893 min, 3.561 min. ¹H NMR (400 MHz, METHANOL-d₄) δ=8.68 (d, J=1.6 Hz, 1H), 8.45 (s, 1H), 8.37 (d, J=7.6 Hz, 1H), 8.00-7.93 (m, 1H), 7.92-7.77 (m, 2H), 7.59-7.48 (m, 3H), 7.47-7.29 (m, 3H), 7.19-7.14 (m, 1H), 4.39 (s, 2H), 4.34-4.21 (m, 2H), 4.03-3.91 (m, 2H), 3.90-3.70 (m, 5H), 3.68-3.51 (m, 6H), 3.49-3.35 (m, 5H), 3.23-3.09 (m, 2H), 3.08-2.98 (m, 2H), 2.95 (s, 3H), 2.84-2.68 (m, 6H), 2.64-2.49 (m, 3H), 2.39-2.26 (m, 1H), 2.23-2.00 (m, 5H), 1.76-1.52 (m, 3H), 1.31-1.27 (m, 3H). ¹⁹F NMR (377 MHz, METHANOL-d₄), δ=−120.70.

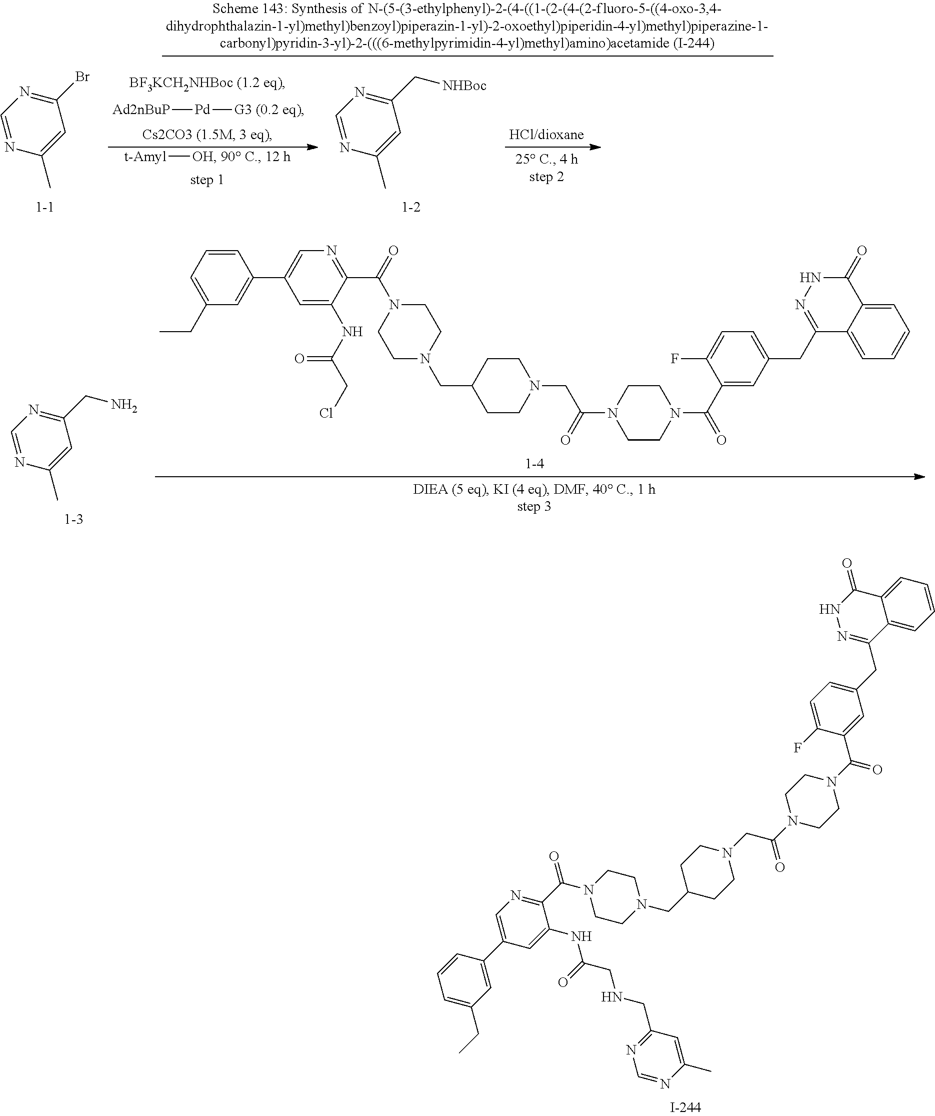

### Step 1: Synthesis of Intermediate 1-2

To a solution of Intermediate 1-1 (100 mg, 578.00 μmol, 1 eq) and potassium;(tert-butoxycarbonylamino)methyl-trifluoro-boranuide (164.43 mg, 693.60 μmol, 1.2 eq) in 2-methyl-2-butanol (2 mL) was added Cs₂CO₃ (1.5 M, 1.16 mL, 3 eq) and Ad₂nBuP Pd G₃ (cataCXium A Pd G₃) (84.19 mg, 115.60 μmol, 0.2 eq). Then the mixture was stirred at 90° C. for 12 h under N₂. The mixture was poured into water (3 mL) and extracted with EA (3 mL*3). The combined organic layers were dried over anhydrous Na₂SO₄, filtered and concentrated to give a residue. The residue was purified by flash silica gel chromatography (ISCO; 12 g SepaFlash Silica Flash Column, Eluent of 0˜70% Ethyl acetate/Petroleum ether gradient @40 mL/min) and the eluent was concentrated to give Intermediate 1-2 (25 mg, 101.89 μmol, 17.63% yield, 91% purity) as yellow oil. LCMS (Method E): Rt=0.399 min, [M+H]⁺=224.0. ¹H NMR (400 MHz, CHLOROFORM-d) δ=9.01 (s, 1H), 7.16 (s, 1H), 5.43 (br s, 1H), 4.39 (br d, J=5.6 Hz, 2H), 2.54 (s, 3H), 1.48 (s, 9H).

### Step 2: Synthesis of Intermediate 1-3

To a mixture of Intermediate 1-2 (25 mg, 101.89 μmol, 1 eq) in DCM (0.5 mL) was added HCl/dioxane (2 M, 0.5 mL), and the mixture was stirred at 25° C. for 4 hrs. The reaction mixture was concentrated under reduced pressure to give Intermediate 1-3 (15 mg, crude, HCl salt) as yellow gum. LCMS (Method B): Rt=0.108 min, [M+H]⁺=124.0.

### Step 3: Synthesis of I-244

To a solution of Intermediate 1-4 (50 mg, 56.15 μmol, 1 eq) and Intermediate 1-3 (10.76 mg, 67.38 μmol, 1.2 eq, HCl) in DMF (1 mL) was added KI (37.29 mg, 224.61 μmol, 4 eq) and DIEA (36.29 mg, 280.76 μmol, 48.90 μL, 5 eq). The mixture was stirred at 40° C. for 1 hr. The reaction mixture was filtered and the mother liquor was purified by prep-HPLC (column: YMC-Actus Triart C18 150*30 mm*7 um; mobile phase: [water (FA)-ACN]; gradient: 10%-40% B over 10 min). The eluent was concentrated under reduced pressure to remove ACN, then lyophilized to afford I-244 (30 mg, 29.73 μmol, 52.95% yield, 96.84% purity) as a yellow solid. LCMS (Method E): Rt=0.450 min, [M+Na]⁺=999.5. ¹H NMR (400 MHz, METHANOL-d₄) δ=9.03 (s, 1H), 8.67 (d, J=1.8 Hz, 1H), 8.59 (s, 1H), 8.39 (d, J=6.8 Hz, 1H), 8.07-7.95 (m, 1H), 7.94-7.78 (m, 2H), 7.63 (s, 1H), 7.58-7.50 (m, 3H), 7.46 (t, J=7.6 Hz, 1H), 7.43-7.32 (m, 2H), 7.21-7.19 (m, 1H), 4.41 (s, 2H), 4.34-4.23 (m, 2H), 4.21 (s, 2H), 3.82 (br s, 6H), 3.77-3.71 (m, 1H), 3.70-3.49 (m, 7H), 3.44-3.36 (m, 3H), 3.17-3.07 (m, 1H), 2.78-2.74 (m, 2H), 2.73-2.63 (m, 4H), 2.59 (s, 3H), 2.51-2.40 (m, 2H), 2.17-1.92 (m, 3H), 1.69-1.50 (m, 2H), 1.33-1.29 (m, 3H). ¹⁹F NMR (377 MHz, METHANOL-d₄), δ=−120.77.

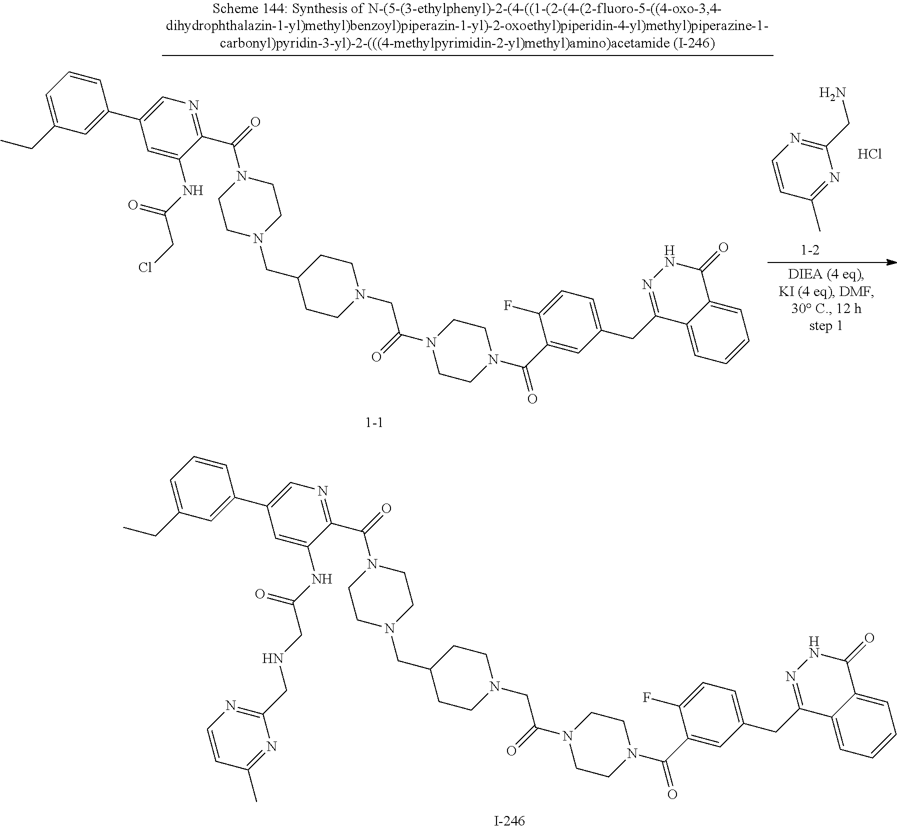

To a solution of Intermediate 1-1 (50 mg, 56.15 μmol, 1 eq) and Intermediate 1-2 (13.44 mg, 84.23 μmol, 1.5 eq, HCl salt) in DMF (0.5 mL) was added DIPEA (29.03 mg, 224.61 μmol, 39.12 μL, 4 eq) and KI (37.29 mg, 224.61 μmol, 4 eq). The reaction mixture was stirred at 30° C. for 12 hrs. The reaction mixture was diluted with MeOH (0.5 mL) and then purified by prep-HPLC (column: Phenomenex luna C18 150*25 mm*10 um; mobile phase: [water (FA)-ACN]; gradient: 2%-32% B over 9 min), and the eluent was concentrated and lyophilized to afford I-246 (17.74 mg, 17.97 μmol, 32.01% yield, 99% purity) as an orange solid. LCMS (Method E): Rt=0.411 min, [M+H]⁺=977.7. ¹H NMR (400 MHz, METHANOL-d₄) δ=8.71-8.61 (m, 3H), 8.38-8.36 (m, 1H), 7.99-7.80 (m, 3H), 7.58-7.48 (m, 4H), 7.46-7.35 (m, 2H), 7.32 (d, J=7.6 Hz, 1H), 7.17-7.16 (m, 1H), 4.70-4.50 (m, 2H), 4.39 (s, 2H), 4.33-4.18 (m, 2H), 4.10 (s, 2H), 3.90-3.65 (m, 7H), 3.58-3.55 (m, 5H), 3.43-3.33 (m, 3H), 3.18-3.02 (m, 2H), 2.76-2.70 (m, 2H), 2.69 (s, 3H), 2.59 (s, 3H), 2.46-2.32 (m, 2H), 2.11-1.88 (m, 3H), 1.65-1.47 (m, 2H), 1.31-1.27 (m, 3H). ¹⁹F NMR (377 MHz, METHANOL-d₄), δ=−120.74.

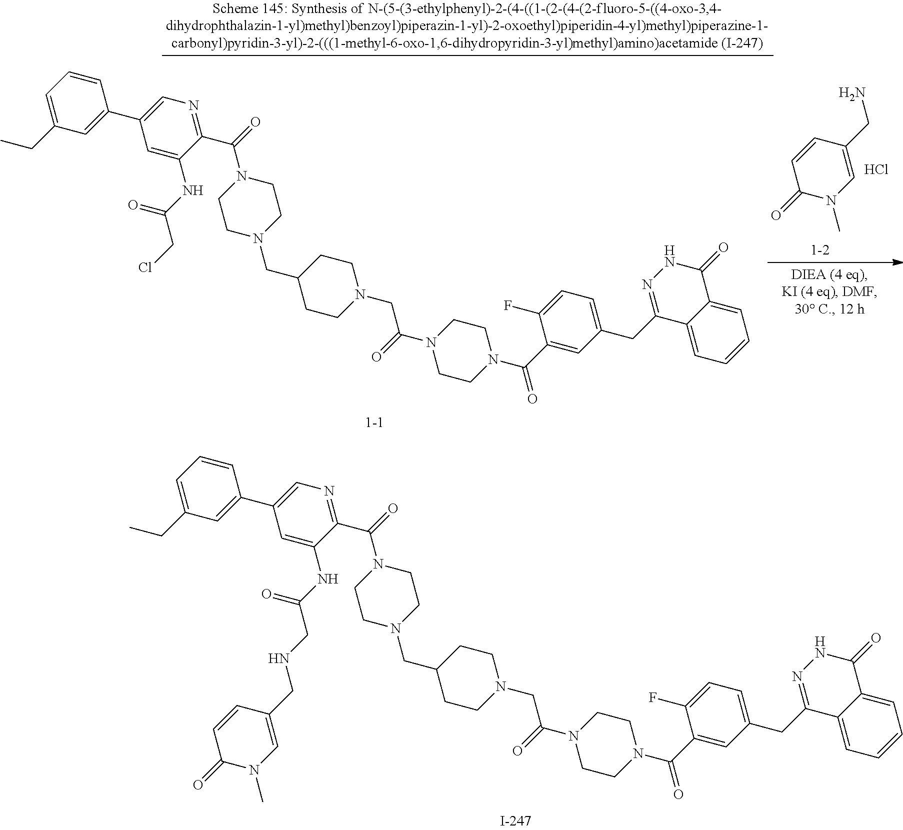

To a solution of Intermediate 1-1 (50 mg, 56.15 μmol, 1 eq) and Intermediate 1-2 (14.71 mg, 84.23 μmol, 1.5 eq, HCl salt) in DMF (0.5 mL) was added KI (37.29 mg, 224.61 μmol, 4 eq) and DIPEA (29.03 mg, 224.61 μmol, 39.12 μL, 4 eq). The reaction mixture was diluted with MeOH (0.5 mL). The mixture was purified by prep-HPLC (column: Phenomenex luna C18 250*50 mm*15 um; mobile phase: [water (FA)-ACN]; gradient: 2%-32% B over 9 min), and the target peak was concentrated and lyophilized to afford I-247 (21.77 mg, 21.72 μmol, 38.69% yield, 99% purity) as a white solid. LCMS (Method E): Rt=0.407 min, [M+H]⁺=992.8. ¹H NMR (400 MHz, METHANOL-d₄) δ=8.73 (s, 1H), 8.62 (d, J=1.6 Hz, 1H), 8.40-8.33 (m, 1H), 8.00-7.71 (m, 5H), 7.56-7.28 (m, 6H), 7.17-7.16 (m, 1H), 6.56 (d, J=9.2 Hz, 1H), 4.39 (s, 2H), 4.30-4.16 (m, 2H), 3.88-3.68 (m, 7H), 3.64-3.48 (m, 11H), 3.39 (s, 3H), 3.16-2.98 (m, 2H), 2.77-2.72 (m, 2H), 2.56 (d, J=16.6 Hz, 4H), 2.36 (d, J=6.8 Hz, 2H), 2.05 (d, J=13.6 Hz, 2H), 1.99-1.88 (m, 1H), 1.63-1.48 (m, 2H), 1.31-1.27 (m, 3H). ¹⁹F NMR (377 MHz, METHANOL-d₄), δ=−120.74.

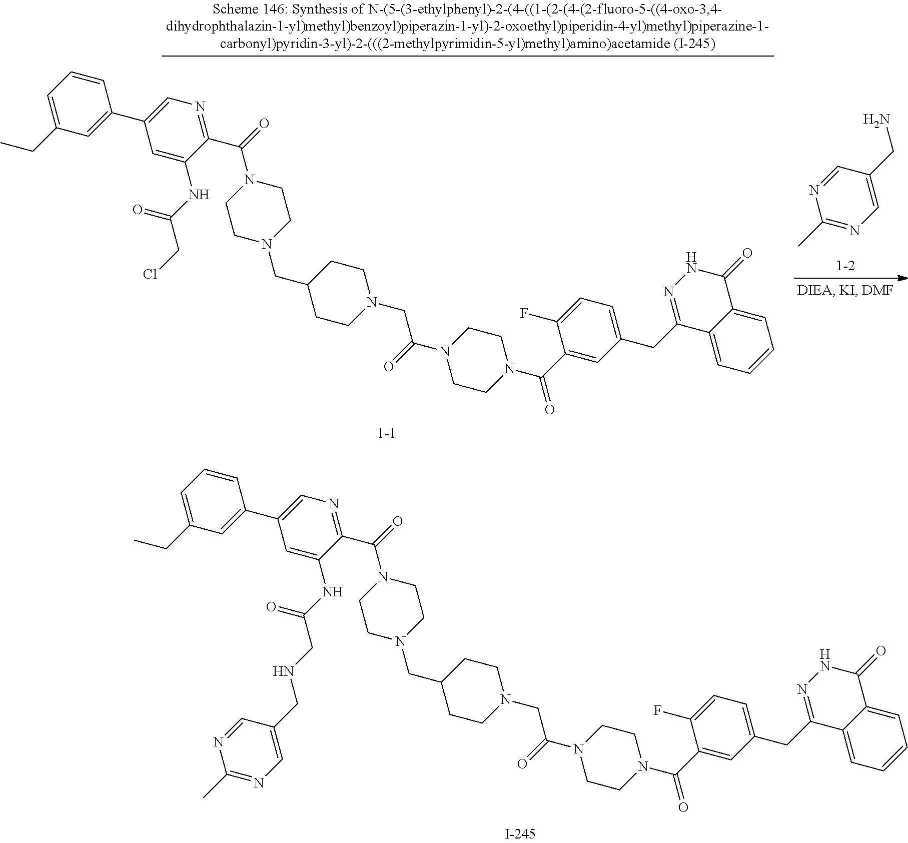

To a solution of Intermediate 1-1 (50 mg, 56.15 μmol, 1 eq) and Intermediate 1-2 (10.37 mg, 84.23 μmol, 1.5 eq) in DMF (1 mL) was added KI (37.29 mg, 224.61 μmol, 4 eq) and DIEA (36.29 mg, 280.76 μmol, 48.90 μL, 5 eq). The mixture was stirred at 40° C. for 1 hr. The reaction mixture was filtered and the filtrate was purified by prep-HPLC (column: Phenomenex luna C18 150*25 mm*10 um; mobile phase: [water (FA)-ACN]; gradient: 18%-48% B over 15 min). The eluent was concentrated under reduced pressure to remove ACN, and then lyophilized to afford I-245 (23 mg, 23.54 μmol, 41.92% yield, 100% purity) as a white solid. LCMS (Method E): Rt=0.430 min, [M+H]⁺=977.6. ¹H NMR (400 MHz, METHANOL-d₄) δ=8.87 (s, 2H), 8.65 (d, J=2.0 Hz, 1H), 8.61-8.53 (m, 1H), 8.40-8.34 (m, 1H), 8.00-7.93 (m, 1H), 7.92-7.79 (m, 2H), 7.59-7.48 (m, 3H), 7.46-7.42 (m, 1H), 7.41-7.29 (m, 2H), 7.19-7.17 (m, 1H), 4.39 (s, 2H), 4.35-4.22 (m, 2H), 4.16-4.06 (m, 2H), 3.99-3.86 (m, 2H), 3.83 (d, J=3.6 Hz, 1H), 3.81-3.68 (m, 6H), 3.68-3.52 (m, 4H), 3.39 (br s, 2H), 3.35 (br s, 2H), 3.21-3.04 (m, 2H), 2.98-2.84 (m, 3H), 2.78-2.72 (m, 3H), 2.71-2.57 (m, 4H), 2.22-2.00 (m, 3H), 1.75-1.49 (m, 2H), 1.31-1.27 (m, 3H). ¹⁹F NMR (377 MHz, METHANOL-d₄), δ=−120.70.

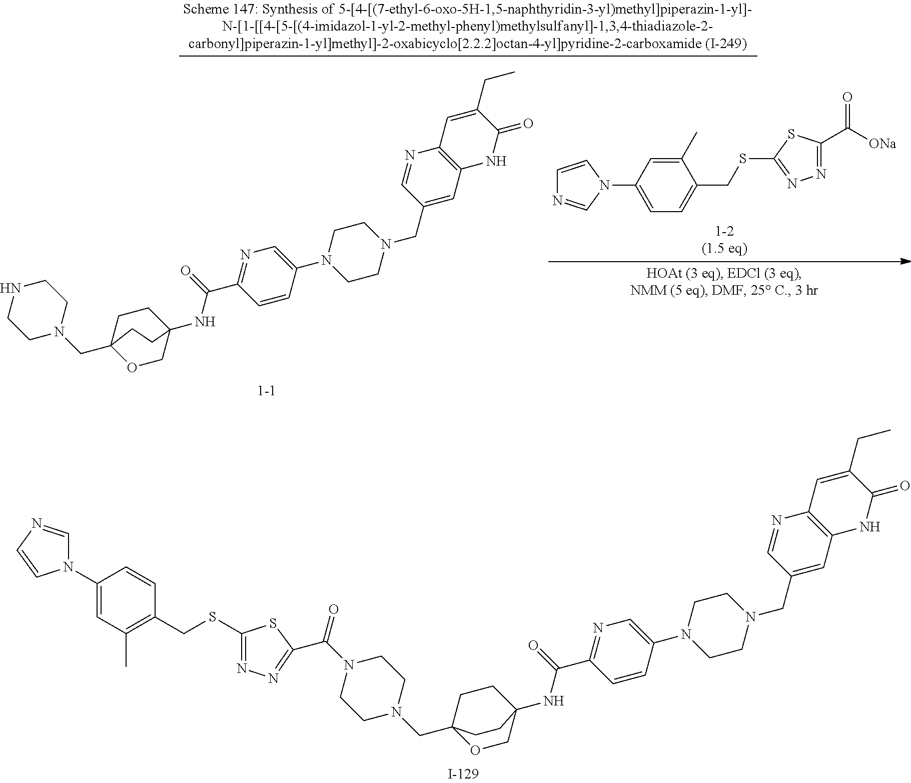

To a solution of intermediate 1-1 (56.51 mg, 94.06 μmol, 1 eq) in DMF (0.5 mL) was added EDCI (54.09 mg, 282.18 μmol, 3 eq.), HOAt (38.41 mg, 282.18 μmol, 3 eq.), NMM (47.57 mg, 470.30 μmol, 51.71 μL, 5 eq.), and intermediate 1-2 (50 mg, 141.09 μmol, 1.5 eq.). The mixture was stirred at 25° C. for 3 hr. The reaction mixture was filtered and then diluted with H₂O (5 mL) and extracted with EA (20 mL*3). The combined organic layers were washed with brine (15 mL*2), dried over anhydrous Na₂SO₄, filtered and concentrated under reduced pressure to give a residue. The residue was purified by prep-HPLC (column: Waters Xbridge 150*25 mm*5 um; mobile phase: [water(NH₄HCO₃)-ACN]; gradient: 388%-68% B over 9 min) to give I-129 (13.47 mg, 14.56 μmol, 15.48% yield, 98.940% purity) as white solid. LCMS (Method E): Retention time: 0.392 min, [M+H]⁺=915.5. ¹H NMR (400 MHz, CHLOROFORM-d) δ=8.57-8.51 (m, 1H), 8.22-8.10 (m, 1H), 8.08-7.97 (m, 1H), 7.87 (d, J=4.4 Hz, 2H), 7.71-7.66 (m, 1H), 7.63-7.59 (m, 1H), 7.56-7.51 (m, 1H), 7.24 (br d, J=2.4 Hz, 1H), 7.23-7.18 (m, 3H), 4.70-4.61 (m, 4H), 4.36-4.26 (m, 1H), 4.14-4.11 (m, 2H), 3.83-3.77 (m, 1H), 3.77-3.64 (m, 3H), 3.40-3.29 (m, 5H), 2.85-2.72 (m, 3H), 2.54-2.44 (m, 11H), 2.34 (s, 3H), 2.31-2.23 (m, 1H), 2.17-1.98 (m, 2H), 1.77-1.63 (m, 1H), 1.33 (s, 1H), 1.32 (s, 3H), 1.30 (s, 1H).

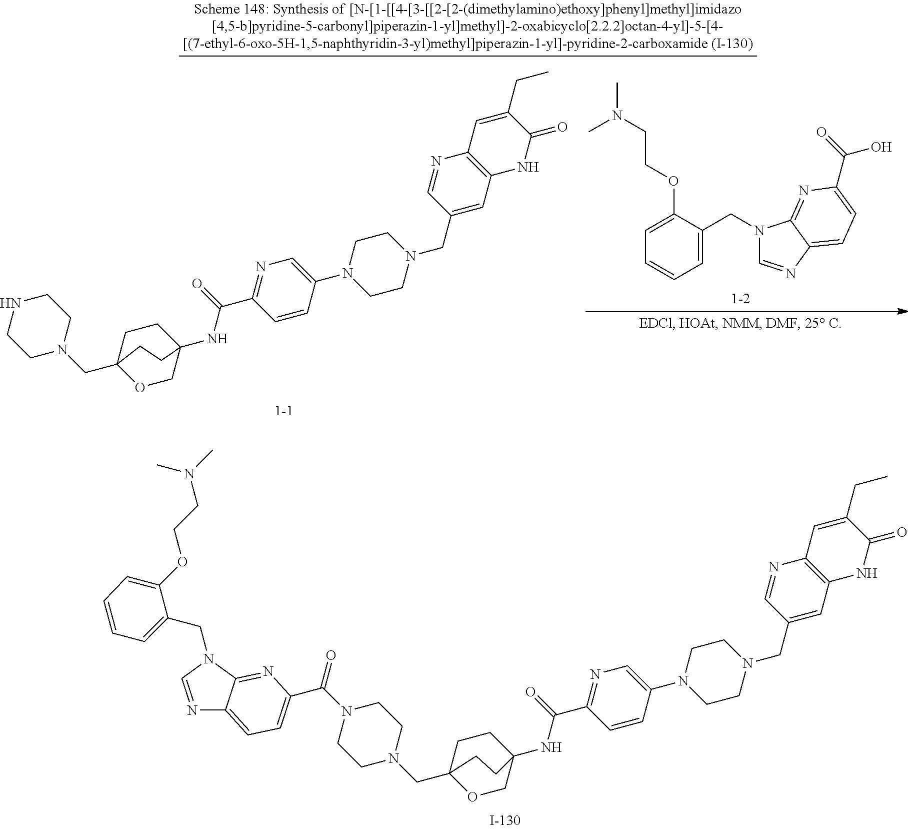

To a solution of 1-1 (24 mg, 69.36 μmol, 1 eq) in DMF (1 mL) was added EDCI (40 mg, 208.07 mol, 3 eq), HOAt (28 mg, 208.07 μmol, 3 eq), NMM (14 mg, 138.71 μmol, 15.25 μL, 2 eq) and intermediate 1-2 (50 mg, 83.23 μmol, 1.2 eq). The mixture was stirred at 25° C. for 12 hr. The reaction solution was filtered and the residue was dissolved in DMF (2 mL) and purified by prep-HPLC (column: Waters Xbridge 150*25 mm*5 um; mobile phase: [water (ammonia hydroxide v/v)-ACN]; gradient: 25%-55% B over 10 min) to give I-130 (21.57 mg, 23.37 μmol, 33.69% yield) as a yellow solid. LCMS (Method G): Retention time: 0.663 min, [M+H]⁺=923.6. ¹H NMR (400 MHz, METHANOL-d4) δ=8.64 (s, 1H), 8.47 (d, J=1.6 Hz, 1H), 8.21 (d, J=2.8 Hz, 1H), 8.13 (d, J=8.4 Hz, 1H), 7.87-7.79 (m, 2H), 7.74 (d, J=2.0 Hz, 1H), 7.56 (d, J=8.4 Hz, 1H), 7.35-7.24 (m, 3H), 7.00 (d, J=8 Hz, 1H), 6.91 (t, J=7.2 Hz, 1H), 5.54 (s, 2H), 4.18-4.07 (m, 4H), 3.78 (s, 2H), 3.70 (s, 2H), 3.46 (s, 2H), 3.40-3.33 (m, 4H), 2.75 (t, J=5.6 Hz, 2H), 2.70-2.60 (m, 8H), 2.50 (s, 2H), 2.34 (s, 5H), 2.32 (s, 1H), 2.11-1.96 (m, 4H), 1.79-1.66 (m, 2H), 1.27 (t, J=7.2 Hz, 3H).

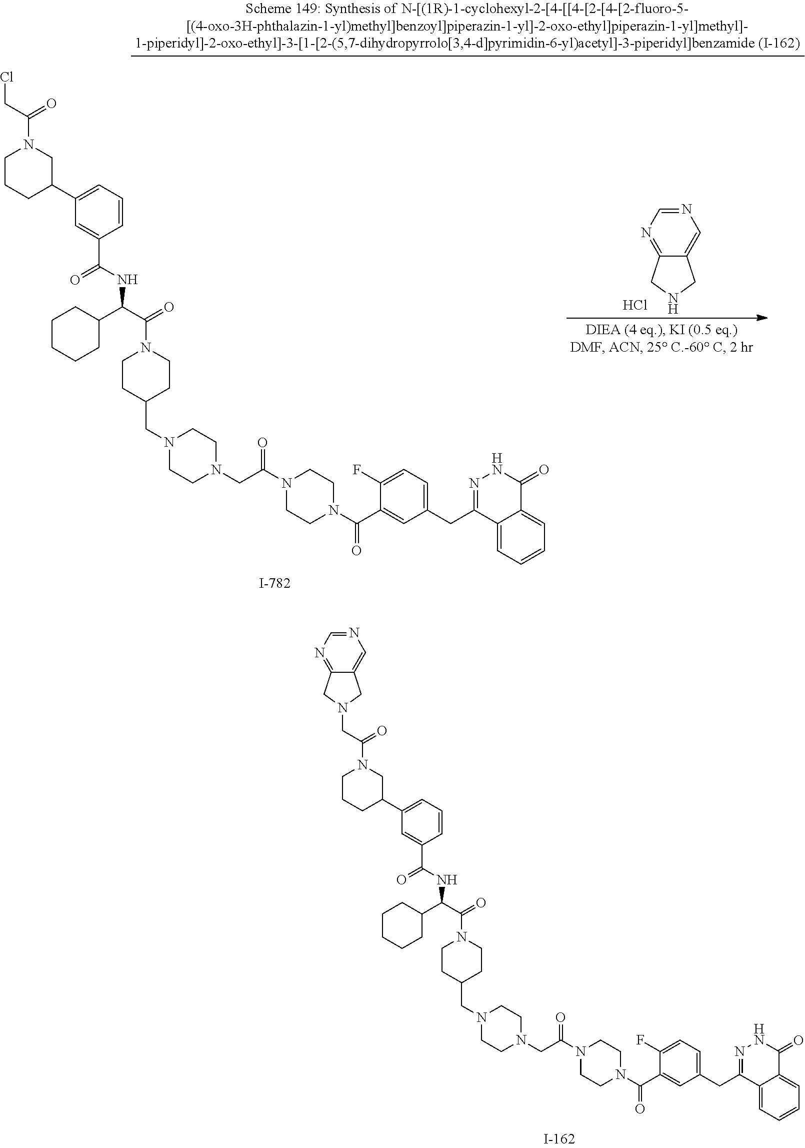 [SMILES: O=C(N[C@@H](C(=O)N1CCC(CN2CCN(CC(=O)N3CCN(C(=O)c4cc(Cc5n[nH]c(=O)c6ccccc56)ccc4F)CC3)CC2)CC1)C1CCCCC1)c1cccc(C2CCCN(C(=O)CCl)C2)c1 ; Cl ; c1ncc2c(n1)CNC2 ; O=C(N[C@@H](C(=O)N1CCC(CN2CCN(CC(=O)N3CCN(C(=O)c4cc(Cc5n[nH]c(=O)c6ccccc56)ccc4F)CC3)CC2)CC1)C1CCCCC1)c1cccc(C2CCCN(C(=O)CN3Cc4cncnc4C3)C2)c1]

To a solution of 6,7-dihydro-5H-pyrrolo[3,4-d]pyrimidine;dihydrochloride (14.66 mg, 75.56 μmol, 1.5 eq) in MeCN (1 mL) and DMF (1 mL) was added DIEA (26.04 mg, 201.49 μmol, 35.10 μL, 4 eq). The reaction mixture was stirred at 25° C. for 0.5 h, then KI (4.18 mg, 25.19 μmol, 0.5 eq) and I-782 (50 mg, 50.37 μmol, 1 eq) were added to the mixture. The reaction mixture was stirred at 60° C. for 4 hr. The reaction mixture was diluted with 0.5 mL of DMF and filtered. The filtrate was used for purification. The mixture was purified by prep-HPLC (column: Waters Xbridge 150*25 mm*5 um; mobile phase: [water (ammonia hydroxide v/v)-ACN]; gradient: 23%-53% B over 10 min) to obtain a solution and then freeze-dried to give I-162 (27.97 mg, 25.82 μmol, 51.26% yield, 99.44% purity) as a yellow solid. LCMS (Method E): Retention time: 0.427 min, [M+H]⁺=1078.0. ¹H NMR (400 MHz, METHANOL-d4) δ=8.98 (s, 1H), 8.63 (s, 1H), 8.37 (d, J=7.6 Hz, 1H), 7.94 (br d, J=6.4 Hz, 1H), 7.91-7.80 (m, 2H), 7.79-7.68 (m, 2H), 7.52-7.35 (m, 4H), 7.16 (t, J=8.8 Hz, 1H), 4.98-4.92 (m, 2H), 4.58 (s, 4H), 4.39 (s, 2H), 4.33-4.26 (m, 1H), 4.23-4.12 (m, 4H), 4.03 (br s, 1H), 3.90-3.70 (m, 5H), 3.67 (br d, J=4.8 Hz, 1H), 3.51 (br s, 2H), 3.26-3.11 (m, 4H), 2.89-2.69 (m, 3H), 2.60-2.41 (m, 7H), 2.21 (br d, J=7.6 Hz, 2H), 2.05 (br d, J=13.6 Hz, 1H), 1.87 (br t, J=9.6 Hz, 6H), 1.80-1.63 (m, 5H), 1.34-1.01 (m, 8H).

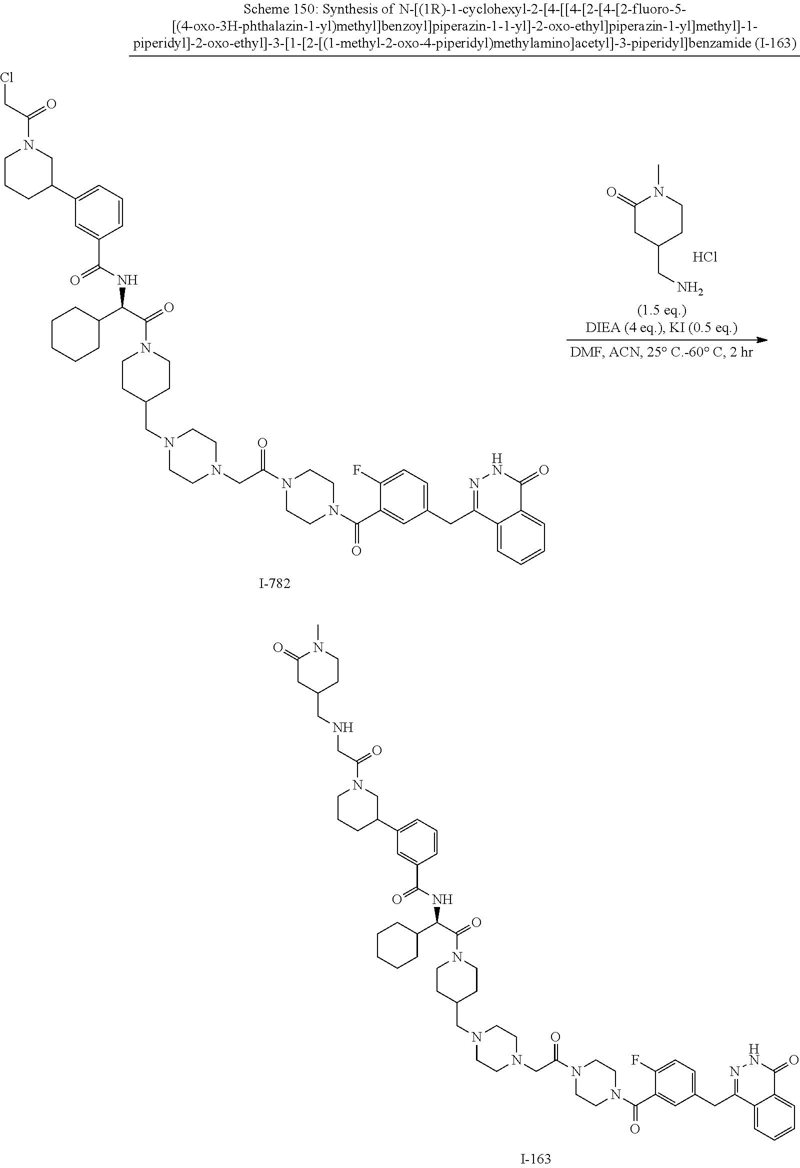

To a solution of 4-(aminomethyl)-1-methyl-piperidin-2-one;hydrochloride (13.50 mg, 75.56 μmol, 1.5 eq) in DMF (1 mL) and ACN (1 mL) was added DIEA (26.04 mg, 201.49 μmol, 35.10 μL, 4 eq), and the reaction mixture was stirred at 25° C. for 0.5 h, then KI (4.18 mg, 25.19 μmol, 0.5 eq) and I-782 (50 mg, 50.37 μmol, 1 eq) were added to the mixture, and the reaction mixture was stirred at 60° C. for 15 hr. The reaction mixture was diluted with 0.5 mL of DMF and filtered. The filtrate was used for purification. The mixture was purified by prep-HPLC (column: Waters Xbridge 150*25 mm*5 um; mobile phase: [water (ammonia hydroxide v/v)-ACN]; gradient: 23%-53% B over 10 min) to obtain a solution and then freeze-dried to give I-163 (10.73 mg, 9.38 μmol, 18.62% yield, 96% purity) as a white solid. LCMS (Method E): Retention time: 0.427 min, [M+H]⁺=1099.0. ¹H NMR (400 MHz, METHANOL-d4) δ=8.37 (br d, J=7.6 Hz, 1H), 7.98-7.92 (m, 1H), 7.91-7.80 (m, 2H), 7.79-7.75 (m, 1H), 7.74-7.68 (m, 1H), 7.52-7.35 (m, 4H), 7.16 (s, 1H), 4.93 (br dd, J=5.6, 8.3 Hz, 2H), 4.58 (br s, 4H), 4.39 (s, 2H), 4.33-4.24 (m, 1H), 3.96-3.84 (m, 1H), 3.82-3.65 (m, 4H), 3.53 (br s, 5H), 3.27-3.10 (m, 4H), 2.92 (br d, J=3.6 Hz, 3H), 2.87-2.63 (m, 4H), 2.60-2.41 (m, 10H), 2.27-2.14 (m, 2H), 2.05 (br d, J=10.4 Hz, 4H), 1.96-1.81 (m, 7H), 1.75-1.51 (m, 5H), 1.17-1.05 (m, 1H), 1.36-1.04 (m, 7H).

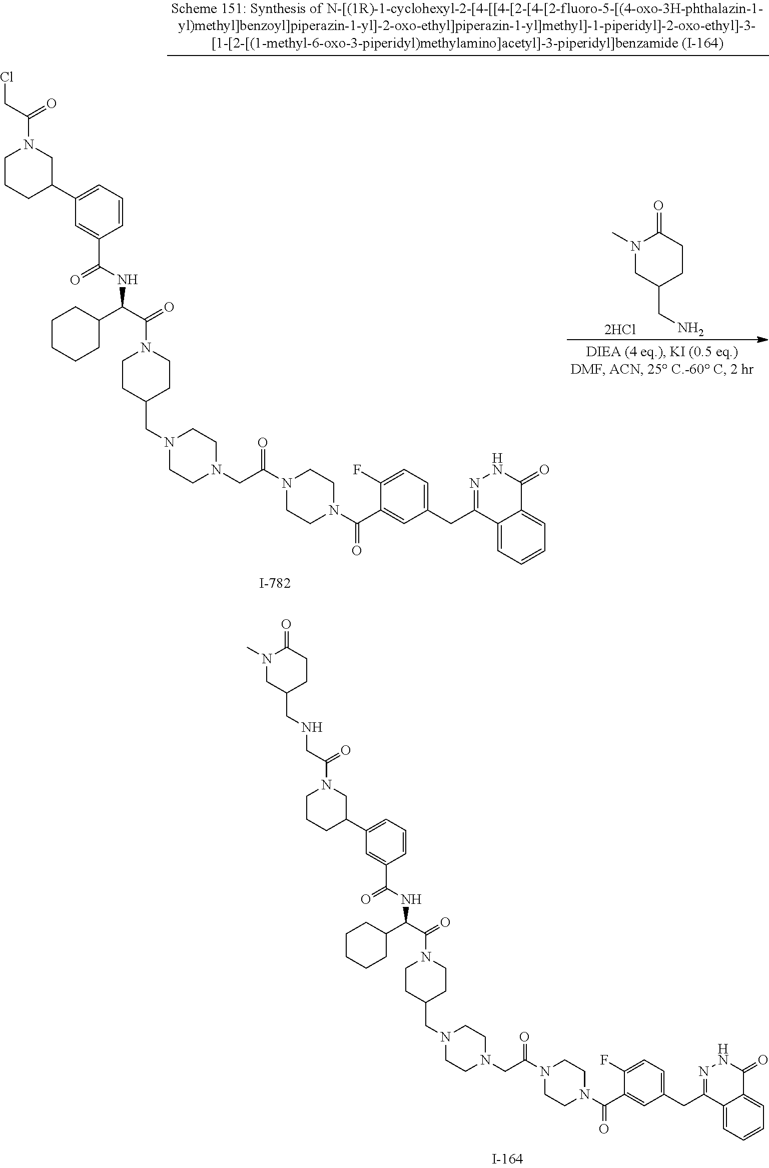

To a solution of 5-(aminomethyl)-1-methyl-piperidin-2-one (16.25 mg, 75.56 μmol, 1.5 eq, 2HCl) m ACN (1 mL) and DMF (1 mL) was added DIEA (26.04 mg, 201.49 μmol, 35.10 μL, 4 eq), and the reaction mixture was stirred at 25° C. for 0.5 h, then KI (4.18 mg, 25.19 μmol, 0.5 eq) and I-782 (50 mg, 50.37 mol, 1 eq) were added to the mixture, the reaction mixture was stirred at 60° C. for 15 hr. The reaction mixture was diluted with 0.5 mL of DMF and filtered. The filtrate was used for purification. The mixture was purified by prep-HPLC (column: Waters Xbridge 150*25 mm*5 um; mobile phase: [water (ammonia hydroxide v/v)-ACN]; gradient: 23%-53% B over 10 min) to obtain a solution and then freeze-dried to give I-164 (13.15 mg, 11.85 μmol, 23.52% yield, 98.944% purity) as a yellow solid. LCMS (Method E): Retention time: 0.418 min, [M+H]⁺=1099.1. ¹H NMR (400 MHz, METHANOL-d4) δ=8.36 (br d, J=7.5 Hz, 1H), 7.94 (br d, J=5.6 Hz, 1H), 7.90-7.80 (m, 2H), 7.79-7.69 (m, 2H), 7.53-7.34 (m, 4H), 7.16 (s, 1H), 4.96 (br s, 1H), 4.64-4.48 (m, 4H), 4.38 (s, 2H), 4.32-4.26 (m, 1H), 3.96-3.85 (m, 1H), 3.82-3.64 (m, 4H), 3.61-3.41 (m, 6H), 3.25-3.05 (m, 4H), 2.92 (d, J=10.0 Hz, 3H), 2.87-2.77 (m, 1H), 2.71 (br s, 2H), 2.64-2.37 (m, 10H), 2.33 (br d, J=7.6 Hz, 3H), 2.08-2.01 (m, 2H), 1.99-1.63 (m, 13H), 1.56-1.46 (m, 1H), 1.28-1.07 (m, 8H).

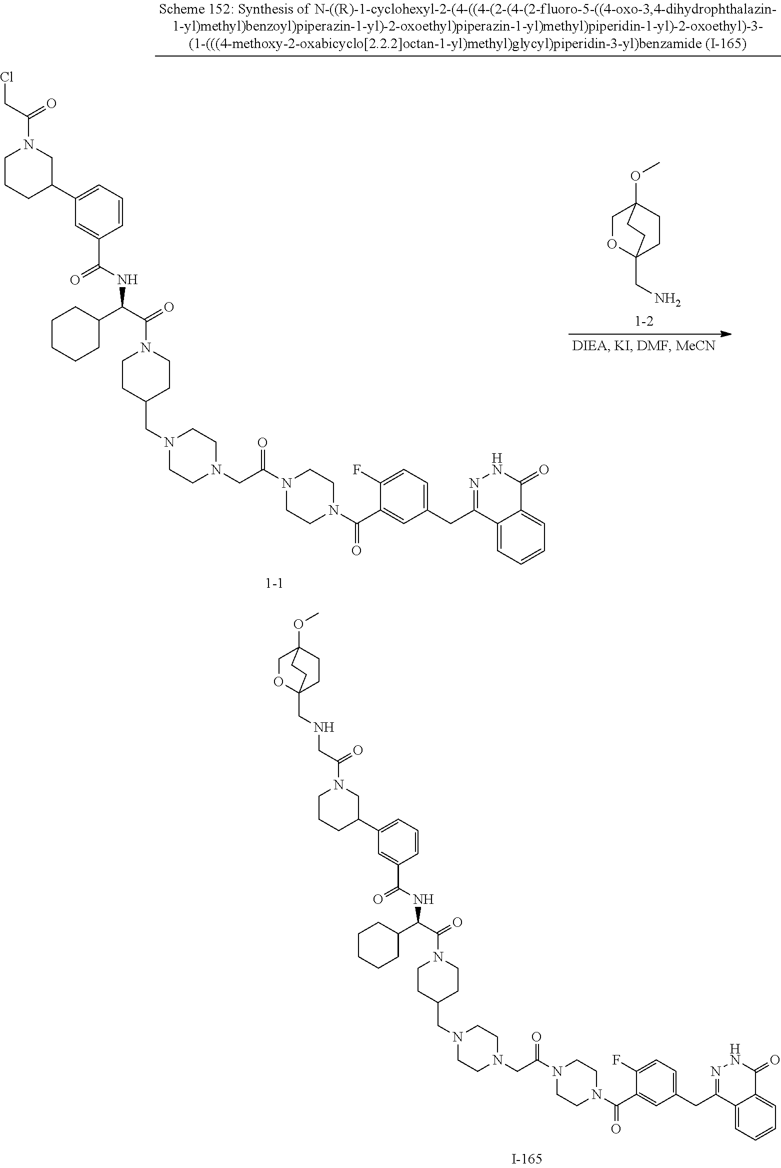 [SMILES: O=C(N[C@@H](C(=O)N1CCC(CN2CCN(CC(=O)N3CCN(C(=O)c4cc(Cc5n[nH]c(=O)c6ccccc56)ccc4F)CC3)CC2)CC1)C1CCCCC1)c1cccc(C2CCCN(C(=O)CCl)C2)c1 ; COC12CCC(CN)(CC1)OC2 ; COC12CCC(CNCC(=O)N3CCCC(c4cccc(C(=O)N[C@@H](C(=O)N5CCC(CN6CCN(CC(=O)N7CCN(C(=O)c8cc(Cc9n[nH]c(=O)c%10ccccc9%10)ccc8F)CC7)CC6)CC5)C5CCCCC5)c4)C3)(CC1)OC2]

To a solution of Intermediate 1-2 (25.88 mg, 151.12 μmol, 2.5 eq) in DMF (1 mL) and MeCN (1 mL) was added DIEA (23.44 mg, 181.34 μmol, 31.59 μL, 3 eq) and it was stirred at 25° C. for 1 hr. Then to the mixture was added KI (5.02 mg, 30.22 μmol, 0.5 eq) and Intermediate 1-1 (60.00 mg, 60.45 μmol, 1 eq) and stirred at 25° C. for 15 hrs. The reaction mixture was filtered to give the filtrate. The filtrate was purified by prep-HPLC (column: C18 150×30 mm; mobile phase: [water (FA)-ACN]; gradient: 10%-40% B over 7 min) to give I-165 (28.65 mg, 24.42 μmol, 40.39% yield, 100% purity, FA salt) as a white solid by lyophilization. LCMS (Method E): Retention time: 0.445 min, [M+H]⁺=1127.6. SFC: Retention time: 4.886 min, 6.676 min. ¹H NMR (400 MHz, METHANOL-d₄) δ=8.52 (s, 1H), 8.39 (d, J=7.6 Hz, 1H), 8.00-7.66 (m, 5H), 7.55-7.33 (m, 4H), 7.19-7.17 (m, 1H), 4.99-4.95 (m, 1H), 4.65-4.51 (m, 2H), 4.41 (s, 2H), 4.32 (br d, J=13.6 Hz, 1H), 4.00-3.67 (m, 9H), 3.65-3.40 (m, 3H), 3.37 (br s, 2H), 3.29-3.16 (m, 5H), 3.08-2.80 (m, 4H), 2.80-2.53 (m, 9H), 2.44-2.30 (m, 2H), 2.12-1.66 (m, 21H), 1.45-1.23 (m, 3H), 1.23-1.00 (m, 4H).

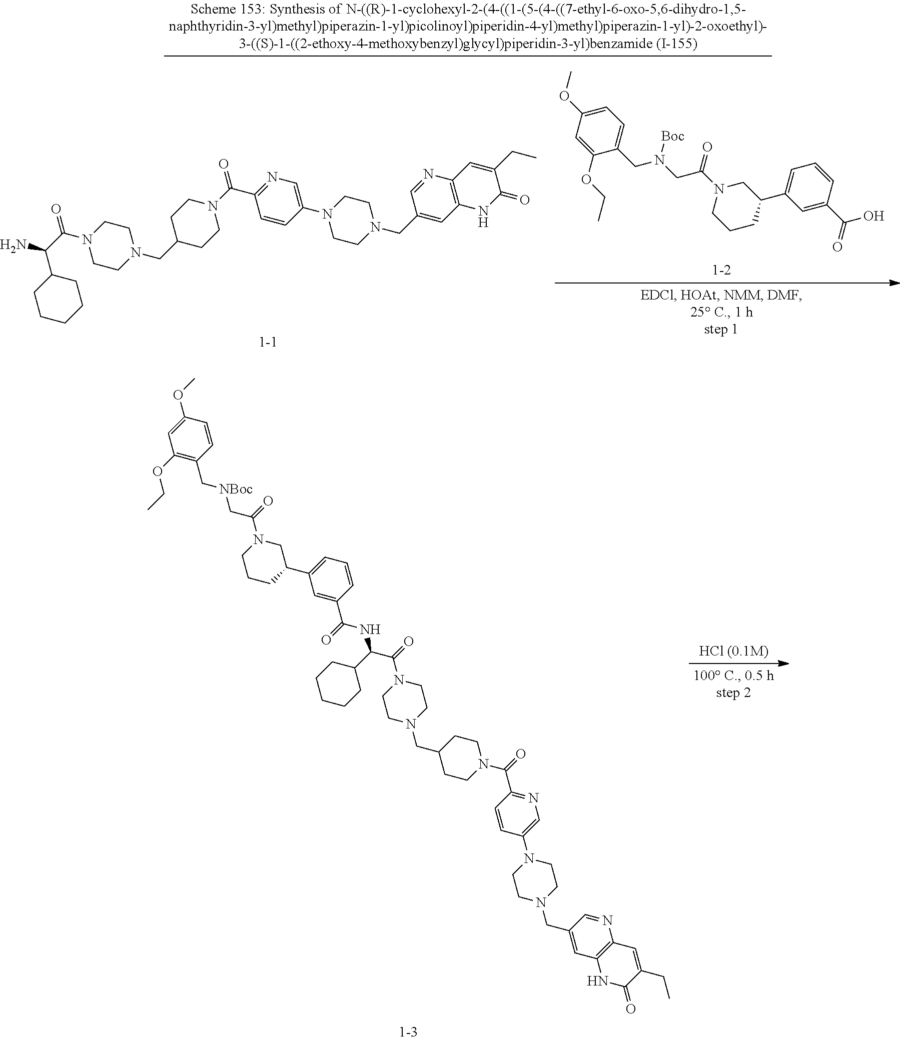

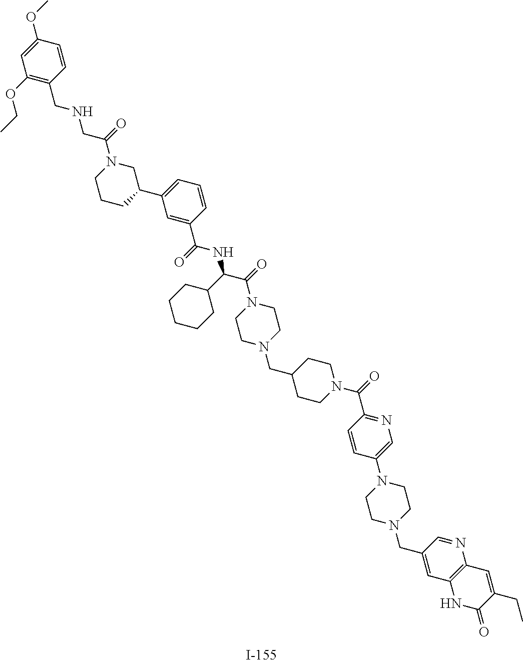 [SMILES: CCOc1cc(OC)ccc1CNCC(=O)N1CCC[C@@H](c2cccc(C(=O)N[C@@H](C(=O)N3CCN(CC4CCN(C(=O)c5ccc(N6CCN(Cc7cnc8cc(CC)c(=O)[nH]c8c7)CC6)cn5)CC4)CC3)C3CCCCC3)c2)C1]

### Step 1: Synthesis of Intermediate 1-3

To a solution of Intermediate 1-1 (90 mg, 128.96 μmol, 1 eq) and Intermediate 1-2 (67.91 mg, 128.96 μmol, 1 eq) in DMF (1 mL) was added EDCI (74.16 mg, 386.87 μmol, 3 eq), NMM (130.44 mg, 1.29 mmol, 141.78 μL, 10 eq) and HOAt (17.55 mg, 128.96 μmol, 1 eq). The mixture was stirred at 25° C. for 1 hr. The reaction mixture was diluted with MeOH (1 mL) and purified by prep-HPLC (FA condition), and the target peak was concentrated and lyophilized to give Intermediate 1-3 (50 mg, 41.44 μmol, 32.14% yield) as a white solid. LCMS (Method E): Rt=0.507 min, [M+H]⁺=1207.3.

### Step 2: Synthesis of I-155

To a solution of Intermediate 1-3 (50 mg, 41.44 μmol, 1 eq) was added HCl (0.1 M, 1.66 mL, 4 eq). The mixture was stirred at 100° C. for 0.5 hr. To the mixture was added NaHCO₃ to adjust the pH to 9˜10, and then lyophilized to give a residue. The residue was purified by prep-HPLC (basic condition), and the target peak was concentrated and lyophilized to give I-155 (16 mg, 14.46 μmol, 34.90% yield, 100% purity) as a white solid. LCMS (Method E): Rt=0.460 min, [M+H]⁺=1106.6. SFC: Rt=3.927 min, 4.796 min, ee %=89.606%. ¹H NMR (400 MHz, METHANOL-d₄) δ=8.50 (d, J=1.6 Hz, 1H), 8.23 (d, J=2.4 Hz, 1H), 7.84 (s, 1H), 7.77 (d, J=1.2 Hz, 1H), 7.75-7.68 (m, 2H), 7.48-7.40 (m, 4H), 7.16-7.11 (m, 1H), 6.54-6.43 (m, 2H), 4.69-4.53 (m, 6H), 4.52 (s, 1H), 4.10-4.01 (m, 2H), 4.00-3.78 (m, 4H), 3.77-3.73 (m, 7H), 3.54-3.46 (m, 2H), 3.40-3.36 (m, 4H), 3.17-3.06 (m, 2H), 2.93-2.83 (m, 1H), 2.80-2.72 (m, 1H), 2.72-2.64 (m, 7H), 2.54-2.38 (m, 4H), 2.29-2.22 (m, 2H), 2.04-2.00 (m, 1H), 1.92-1.84 (m, 4H), 1.83-1.73 (m, 4H), 1.69 (d, J=11.6 Hz, 2H), 1.63-1.52 (m, 1H), 1.46-1.38 (m, 3H), 1.31-1.26 (m, 5H), 1.24 (d, J=9.6 Hz, 2H), 1.10 (d, J=12.0 Hz, 2H).

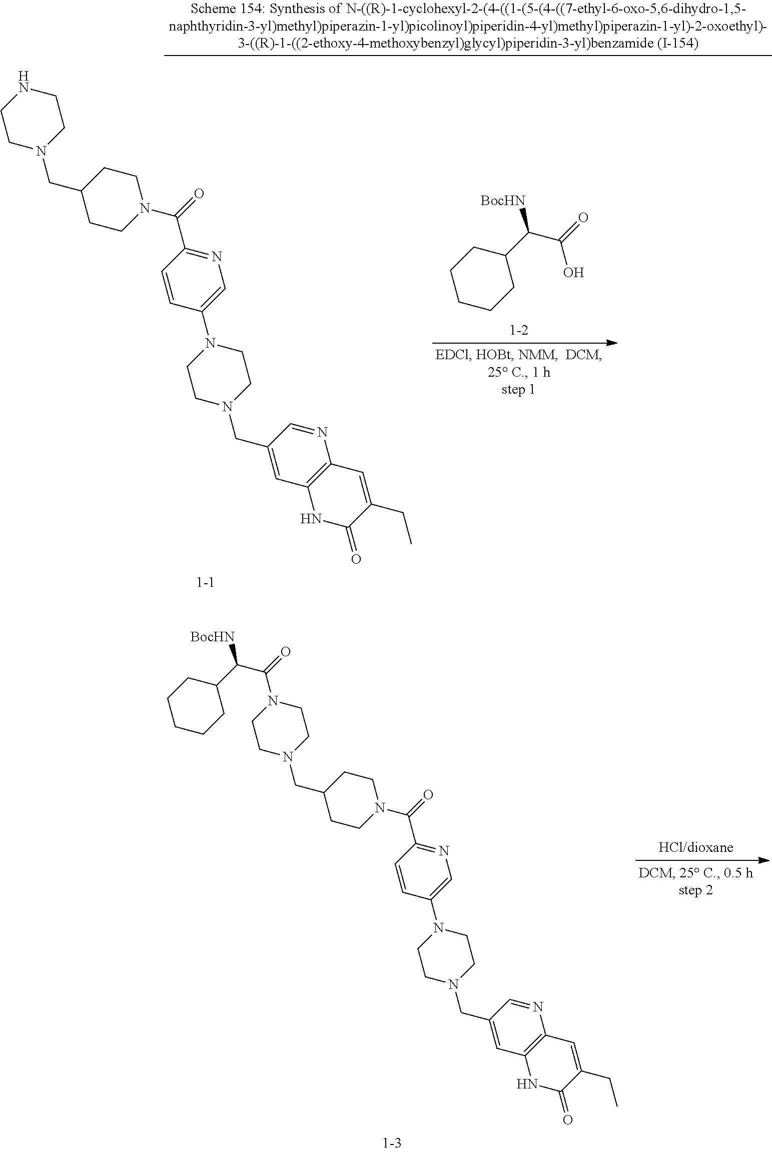 [SMILES: CCc1cc2ncc(CN3CCN(c4ccc(C(=O)N5CCC(CN6CCNCC6)CC5)nc4)CC3)cc2[nH]c1=O ; CC(C)(C)OC(=O)N[C@@H](C(=O)O)C1CCCCC1 ; CCc1cc2ncc(CN3CCN(c4ccc(C(=O)N5CCC(CN6CCN(C(=O)[C@H](NC(=O)OC(C)(C)C)C7CCCCC7)CC6)CC5)nc4)CC3)cc2[nH]c1=O]

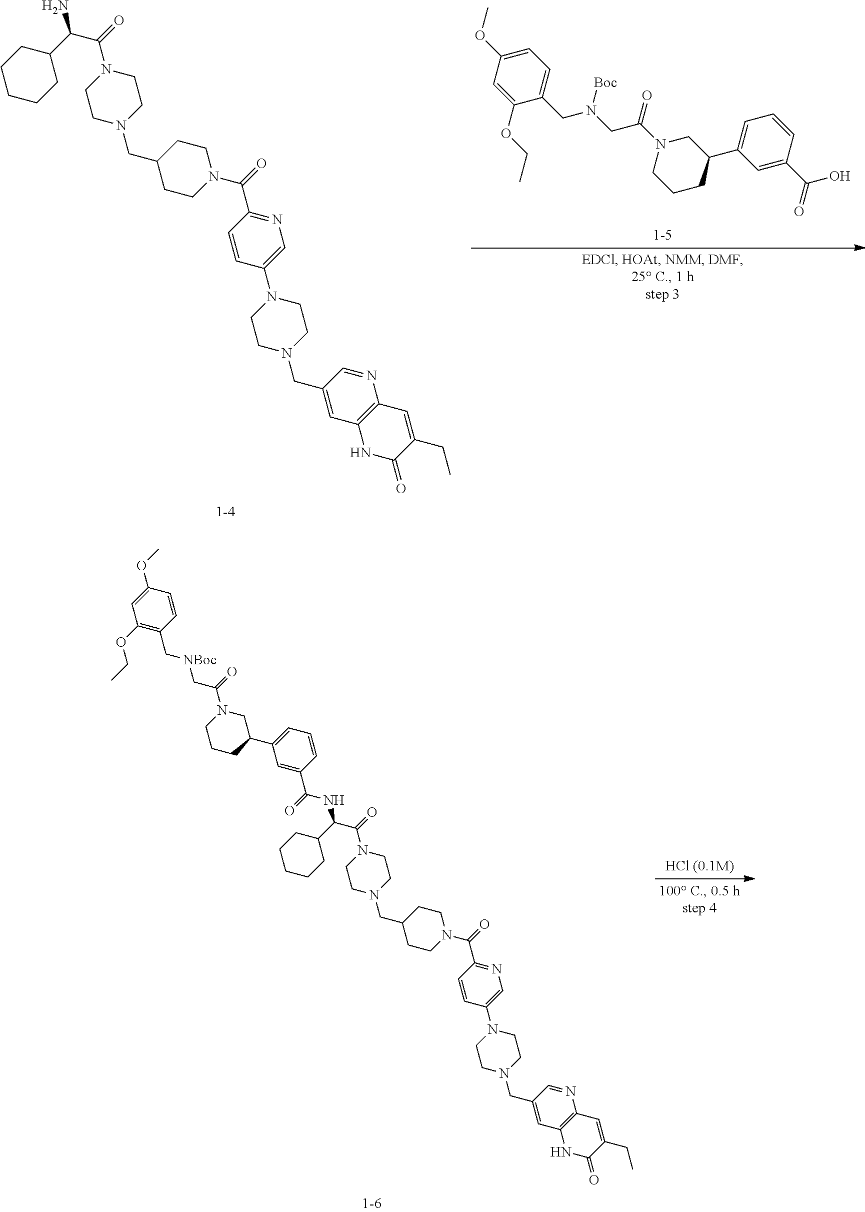 [SMILES: CCc1cc2ncc(CN3CCN(c4ccc(C(=O)N5CCC(CN6CCN(C(=O)[C@H](N)C7CCCCC7)CC6)CC5)nc4)CC3)cc2[nH]c1=O ; CCOc1cc(OC)ccc1CN(CC(=O)N1CCC[C@H](c2cccc(C(=O)O)c2)C1)C(=O)OC(C)(C)C ; CCOc1cc(OC)ccc1CN(CC(=O)N1CCC[C@H](c2cccc(C(=O)N[C@@H](C(=O)N3CCN(CC4CCN(C(=O)c5ccc(N6CCN(Cc7cnc8cc(CC)c(=O)[nH]c8c7)CC6)cn5)CC4)CC3)C3CCCCC3)c2)C1)C(=O)OC(C)(C)C]

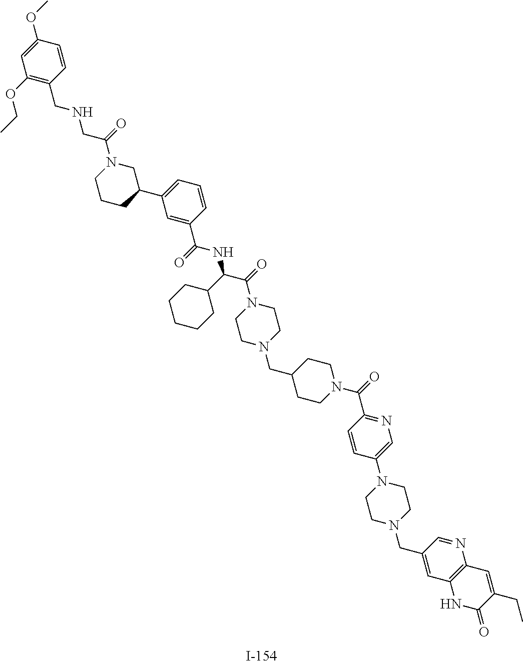 [SMILES: CCOc1cc(OC)ccc1CNCC(=O)N1CCC[C@H](c2cccc(C(=O)N[C@@H](C(=O)N3CCN(CC4CCN(C(=O)c5ccc(N6CCN(Cc7cnc8cc(CC)c(=O)[nH]c8c7)CC6)cn5)CC4)CC3)C3CCCCC3)c2)C1]

### Step 1: Synthesis of Intermediate 1-3

To a solution of Intermediate 1-1 (150 mg, 252.03 μmol, 1 eq, HCl salt) and Intermediate 1-2 (64.85 mg, 252.03 μmol, 1 eq) in DCM (1 mL) was added EDCI (144.94 mg, 756.08 μmol, 3 eq), NMM (254.92 mg, 2.52 mmol, 277.08 μL, 10 eq) and HOBt (34.05 mg, 252.03 μmol, 1 eq). The mixture was stirred at 25° C. for 1 hr. The reaction mixture was quenched with water (5 mL). The mixture was extracted with EA (5 mL*3) and dried over anhydrous Na₂SO₄. The mixture was filtered and the filtrate was concentrated under vacuum. The residue was purified by prep-HPLC (FA condition) to afford Intermediate 1-3 (70 mg, 87.72 μmol, 34.80% yield, 100% purity) as a white solid. LCMS (Method E): Rt=0.477 min, [M+H]⁺=798.2. SFC: Rt=1.974 min.

### Step 2: Synthesis of Intermediate 1-4

To a solution of Intermediate 1-3 (70 mg, 87.72 μmol, 1 eq) in DCM (0.5 mL) was added HCl/dioxane (0.5 mL). The mixture was stirred at 25° C. for 0.5 hr. The reaction mixture was concentrated under the pressure to give Intermediate 1-4 (70 mg, crude) as a white solid. LCMS (Method E): Rt=0.380 min, [M+H]⁺=698.4.

### Step 3: Synthesis of Intermediate 1-6

To a solution of Intermediate 1-4 (60 mg, 85.97 μmol, 1 eq) and Intermediate 1-5 (45.27 mg, 85.97 μmol, 1 eq) in DMF (2 mL) was added EDCI (49.44 mg, 257.91 μmol, 3 eq), NMM (86.96 mg, 859.71 μmol, 94.52 μL, 10 eq) and HOAt (11.70 mg, 85.97 μmol, 1 eq). The mixture was stirred at 25° C. for 1 hr. The reaction mixture was diluted with MeOH (1 mL) and purified by prep-HPLC (FA condition), then concentrated to remove organic solvents and lyophilized to give Intermediate 1-6 (60 mg, 49.73 μmol, 57.85% yield) as a white solid. LCMS (Method E): Rt=0.504 min, [M+H]⁺=1207.2.

### Step 4: Synthesis of I-154

To a solution of Intermediate 1-6 (60 mg, 49.73 μmol, 1 eq) was added HCl (0.1 M, 1.99 mL, 4 eq). The mixture was stirred at 100° C. for 0.5 hr. To the mixture was added NaHCO₃ to adjust the pH to 9˜10, and then lyophilized to give a residue. The residue was purified by prep-HPLC (basic condition), then the target peak was concentrated to remove organic solvents and lyophilized to give I-154 (14.19 mg, 12.13 μmol, 24.40% yield, 94.598% purity) as a white solid. LCMS (Method E): Rt=0.450 min, [M+H]⁺=1106.6. SFC: Rt=0.909 min, 1.223 min, ee %=95.77%. ¹H NMR (400 MHz, METHANOL-d₄) δ=8.50 (d, J=1.6 Hz, 1H), 8.23 (d, J=2.4 Hz, 1H), 7.85-7.68 (m, 4H), 7.48-7.40 (m, 4H), 7.17-7.12 (m, 1H), 6.55-6.42 (m, 2H), 4.58 (s, 6H), 4.54-4.49 (m, 1H), 4.09-4.03 (m, 2H), 3.93-3.79 (m, 4H), 3.78 (s, 3H), 3.75 (d, J=8.8 Hz, 4H), 3.54 (d, J=6.4 Hz, 2H), 3.40-3.35 (m, 4H), 3.20-3.05 (m, 2H), 2.94-2.82 (m, 1H), 2.76-2.63 (m, 8H), 2.56-2.37 (m, 4H), 2.31-2.20 (m, 2H), 2.07-2.00 (m, 1H), 1.92.

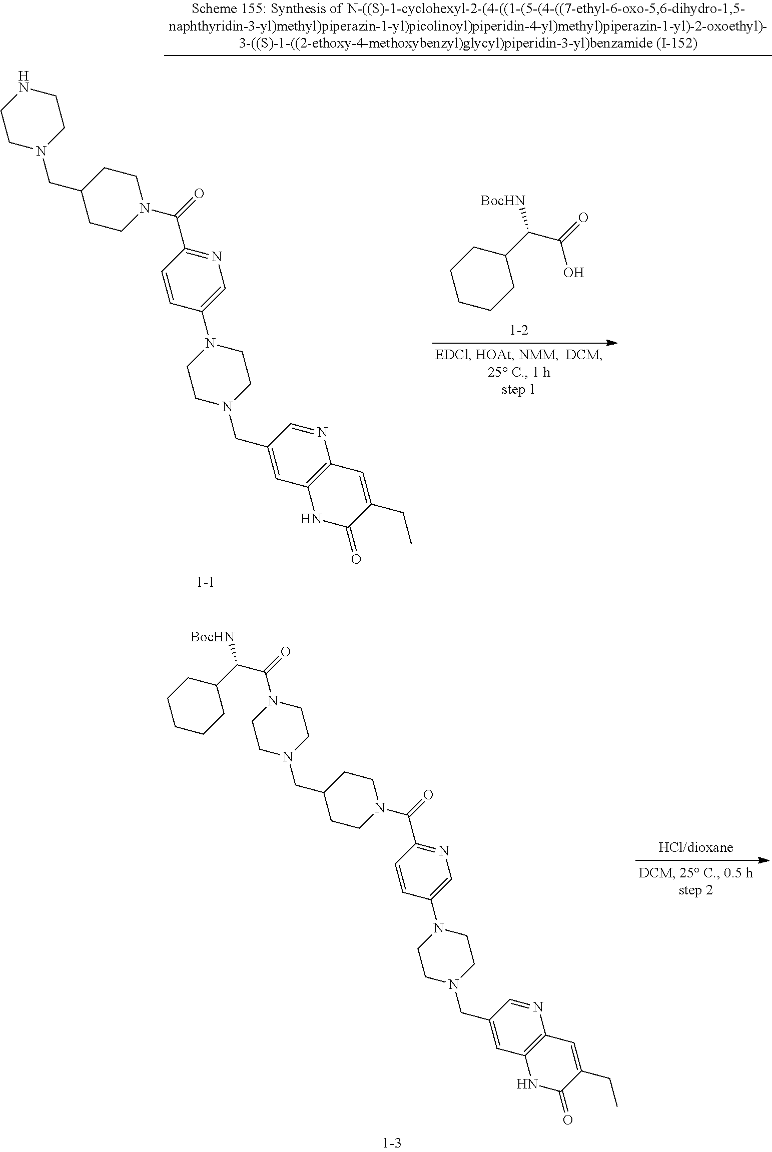 [SMILES: CCc1cc2ncc(CN3CCN(c4ccc(C(=O)N5CCC(CN6CCNCC6)CC5)nc4)CC3)cc2[nH]c1=O ; CC(C)(C)OC(=O)N[C@H](C(=O)O)C1CCCCC1 ; CCc1cc2ncc(CN3CCN(c4ccc(C(=O)N5CCC(CN6CCN(C(=O)[C@@H](NC(=O)OC(C)(C)C)C7CCCCC7)CC6)CC5)nc4)CC3)cc2[nH]c1=O]

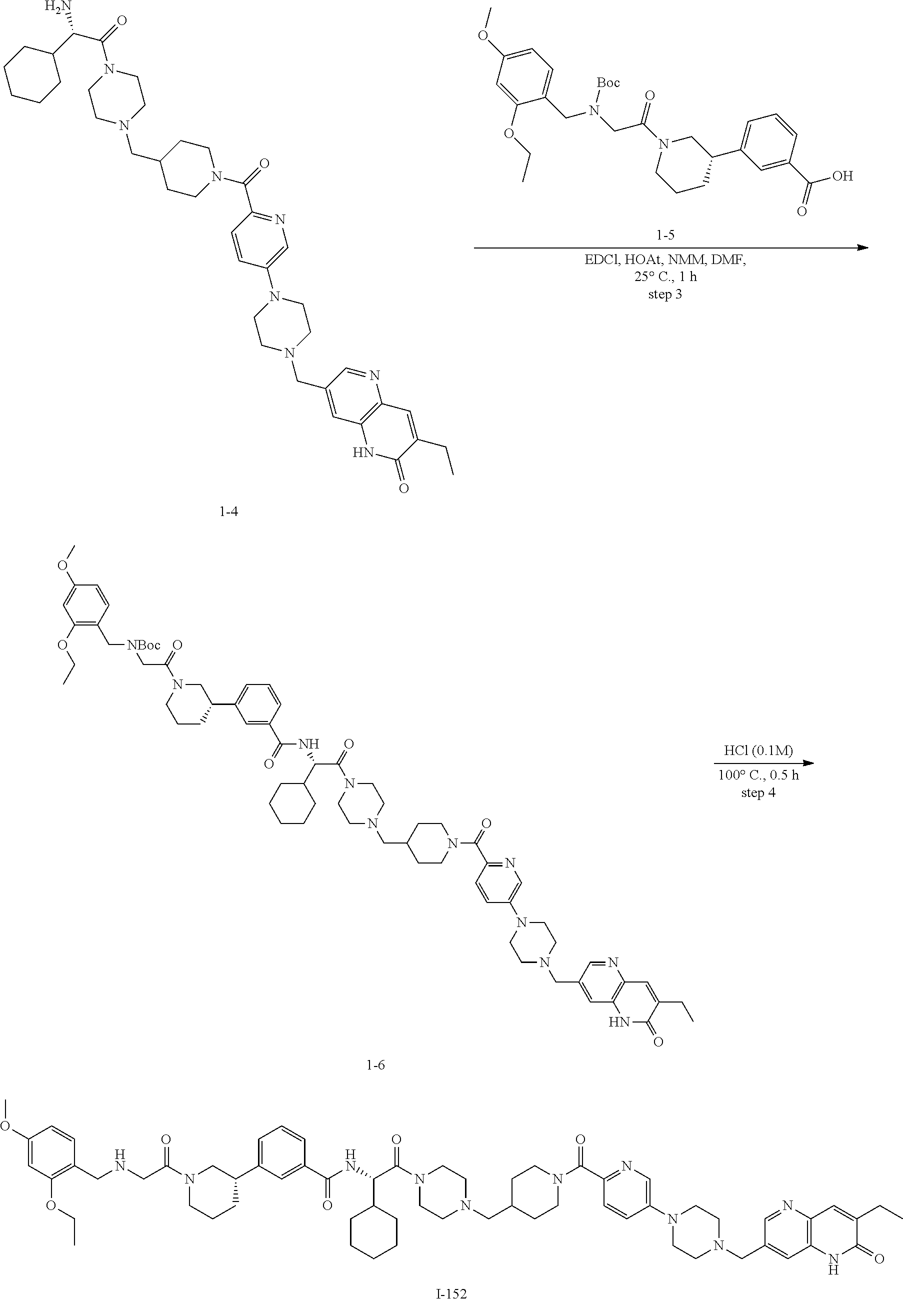

### Step 1: Synthesis of Intermediate 1-3

To a solution of Intermediate 1-1 (80 mg, 134.41 μmol, 1 eq, HCl salt) and Intermediate 1-2 (38.05 mg, 147.86 μmol, 1.1 eq) in DCM (1 mL) was added EDCI (77.30 mg, 403.24 μmol, 3 eq), NMM (135.96 mg, 1.34 mmol, 147.78 μL, 10 eq) and HOAt (18.30 mg, 134.41 μmol, 18.80 μL, 1 eq). The mixture was stirred at 25° C. for 1 hr. The reaction mixture was diluted with MeOH (1 mL) and purified by prep-HPLC (FA condition), and the target peak was concentrated and lyophilized to give Intermediate 1-3 (100 mg, 120.30 μmol, 89.50% yield, 96% purity) as a white solid. LCMS (Method E): Rt=0.420 min, [M+H]⁺=798.7.

### Step 2: Synthesis of Intermediate 1-4

To a solution of Intermediate 1-3 (100 mg, 125.31 μmol, 1 eq) in DCM (0.5 mL) was added HCl/dioxane (0.5 mL). The mixture was stirred at 25° C. for 0.5 hr. The reaction mixture was concentrated under the pressure to give Intermediate 1-4 (90 mg, crude, HCl salt) as a white solid. LCMS (Method E): Rt=0.343 min, [M+H]⁺=698.6.

### Step 3: Synthesis of Intermediate 1-6

To a solution of Intermediate 1-4 (90 mg, 128.96 μmol, 1 eq) and Intermediate 1-5 (67.91 mg, 128.96 μmol, 1 eq) in DMF (1 mL) was added EDCI (74.16 mg, 386.87 μmol, 3 eq), NMM (130.44 mg, 1.29 mmol, 141.78 μL, 10 eq) and HOAt (17.55 mg, 128.96 μmol, 18.04 μL, 1 eq). The mixture was stirred at 25° C. for 1 hr. The reaction mixture was diluted with MeOH (1 mL) and purified by prep-HPLC (FA condition), and the target peak was concentrated and lyophilized to give Intermediate 1-6 (50 mg, 41.44 μmol, 32.14% yield, 1000% purity) as a white solid. LCMS (Method E): Rt=0.502 min, [M+H]⁺=1207.2.

### Step 4: Synthesis of I-152

A solution of Intermediate 1-6 (50 mg, 41.44 μmol, 1 eq) was added HCl (0.1 M, 1.66 mL, 4 eq) was stirred at 100° C. for 0.5 hr. To the mixture was added NaHCO₃ to adjust the pH to 9˜10, and then lyophilized to give a residue. The residue was purified by prep-HPLC (basic condition) and lyophilized to afford I-152 (20 mg, 18.08 μmol, 43.62% yield, 100% purity) as a white solid. LCMS (Method E): Rt=0.449 min, [M+H]⁺=1107.8. SFC: Rt=4.926 min. ¹H NMR (400 MHz, METHANOL-d₄) δ=8.50 (d, J=1.6 Hz, 1H), 8.23 (d, J=2.4 Hz, 1H), 7.84 (s, 1H), 7.77 (s, 1H), 7.75-7.67 (m, 2H), 7.48-7.40 (m, 4H), 7.16-7.12 (m, 1H), 6.55-6.43 (m, 2H), 4.58 (s, 7H), 4.11-4.02 (m, 2H), 3.96-3.79 (m, 4H), 3.78-3.74 (m, 7H), 3.57-3.51 (m, 2H), 3.39-3.36 (m, 4H), 3.18-3.05 (m, 2H), 2.95-2.81 (m, 1H), 2.75-2.63 (m, 8H), 2.54-2.37 (m, 4H), 2.25 (d, J=7.2 Hz, 2H), 2.09-2.00 (m, 1H), 1.91-1.84 (m, 4H), 1.83-1.72 (m, 4H), 1.72-1.66 (m, 2H), 1.59 (d, J=4.4 Hz, 1H), 1.46-1.38 (m, 3H), 1.30-1.26 (m, 5H), 1.24-1.18 (m, 2H), 1.16-1.08 (m, 2H).

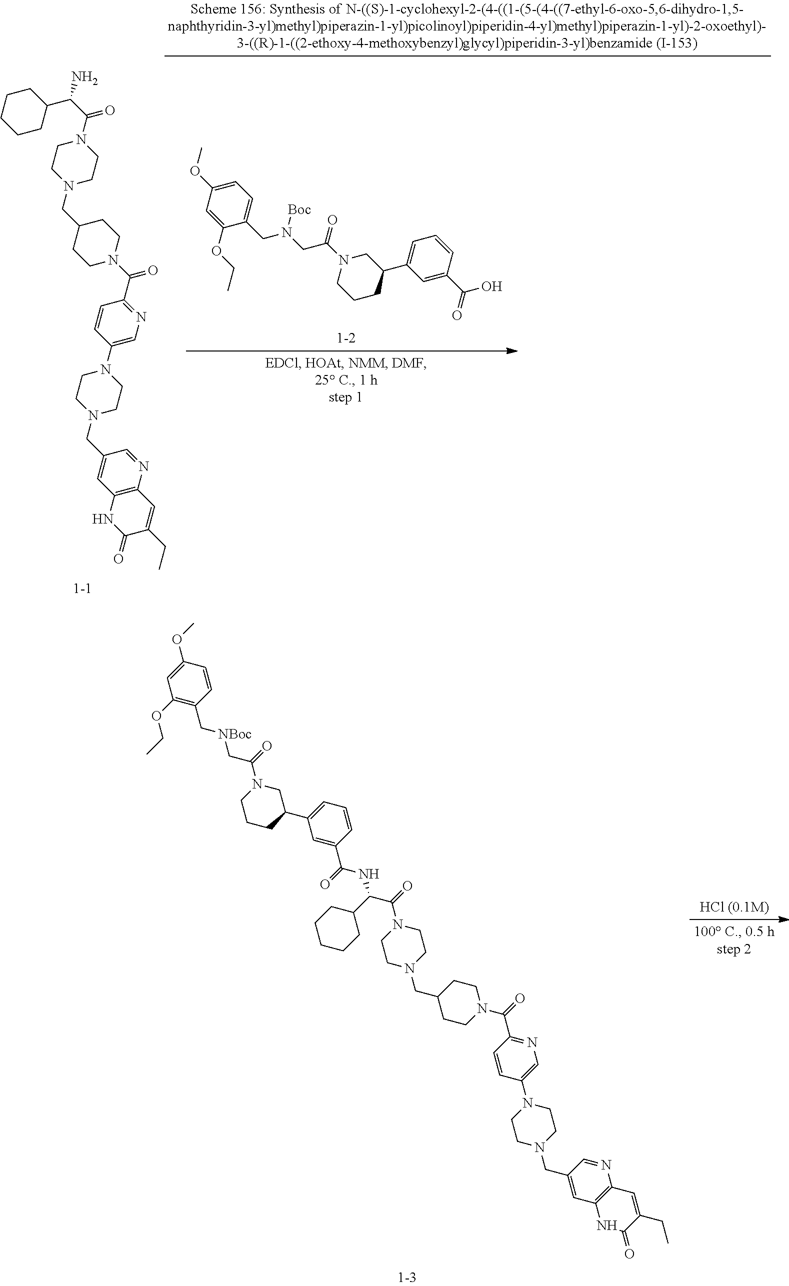 [SMILES: CCc1cc2ncc(CN3CCN(c4ccc(C(=O)N5CCC(CN6CCN(C(=O)[C@@H](N)C7CCCCC7)CC6)CC5)nc4)CC3)cc2[nH]c1=O ; CCOc1cc(OC)ccc1CN(CC(=O)N1CCC[C@H](c2cccc(C(=O)O)c2)C1)C(=O)OC(C)(C)C ; CCOc1cc(OC)ccc1CN(CC(=O)N1CCC[C@H](c2cccc(C(=O)N[C@H](C(=O)N3CCN(CC4CCN(C(=O)c5ccc(N6CCN(Cc7cnc8cc(CC)c(=O)[nH]c8c7)CC6)cn5)CC4)CC3)C3CCCCC3)c2)C1)C(=O)OC(C)(C)C]

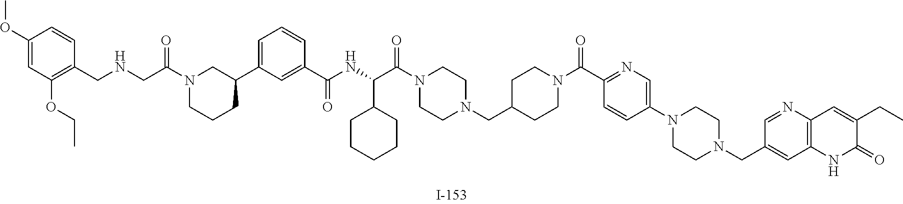 [SMILES: CCOc1cc(OC)ccc1CNCC(=O)N1CCC[C@H](c2cccc(C(=O)N[C@H](C(=O)N3CCN(CC4CCN(C(=O)c5ccc(N6CCN(Cc7cnc8cc(CC)c(=O)[nH]c8c7)CC6)cn5)CC4)CC3)C3CCCCC3)c2)C1]

### Step 1: Synthesis of Intermediate 1-3

To a solution of Intermediate 1-1 (60 mg, 85.97 μmol, 1 eq) and Intermediate 1-2 (45.27 mg, 85.97 μmol, 1 eq) in DMF (1 mL) was added EDCI (49.44 mg, 257.91 μmol, 3 eq), NMM (86.96 mg, 859.71 μmol, 94.52 μL, 10 eq) and HOAt (11.70 mg, 85.97 gmol, 1 eq). The mixture was stirred at 25° C. for 1 hr. The reaction mixture was diluted with MeOH (1 mL) and purified by prep-HPLC (FA condition) to afford Intermediate 1-3 (60 mg, 49.73 μmol, 57.85% yield) as a white solid. LCMS (Method E): Rt=0.522 min, [M+H]⁺=1207.7.

### Step 2: Synthesis of I-153

To a solution of Intermediate 1-3 (60 mg, 49.73 μmol, 1 eq) was added HCl (0.1 M, 1.99 mL, 4 eq). The mixture was stirred at 100° C. for 0.5 hr. To the mixture was added NaHCO₃ to adjust the pH to 9˜10, and then lyophilized to give a residue. The residue was purified by prep-HPLC (basic condition) and lyophilized to afford I-153 (16.85 mg, 15.23 gmol, 30.62% yield, 1000% purity) as a white solid. LCMS (Method E): Rt=0.440 min, [M+H]⁺=1106.6. SFC: Rt=1.750 min. ¹H NMR (400 MHz, METHANOL-d₄) δ=8.51 (d, J=1.6 Hz, 1H), 8.25 (d, J=2.4 Hz, 1H), 7.86 (s, 1H), 7.80-7.68 (m, 3H), 7.52-7.41 (m, 4H), 7.20-7.15 (m, 1H), 6.57-6.47 (m, 2H), 4.60 (s, 7H), 4.13-4.04 (m, 2H), 3.92-3.81 (m, 4H), 3.81-3.74 (m, 7H), 3.59 (d, J=5.6 Hz, 2H), 3.42-3.37 (m, 4H), 3.20-3.08 (m, 2H), 2.89-2.65 (m, 9H), 2.57-2.39 (m, 4H), 2.27 (d, J=6.4 Hz, 2H), 2.06 (s, 1H), 1.94-1.86 (m, 4H), 1.85-1.74 (m, 4H), 1.74-1.67 (m, 2H), 1.66-1.53 (m, 1H), 1.49-1.40 (m, 3H), 1.33-1.27 (m, 5H), 1.27-1.21 (m, 2H), 1.18-1.09 (m, 2H).

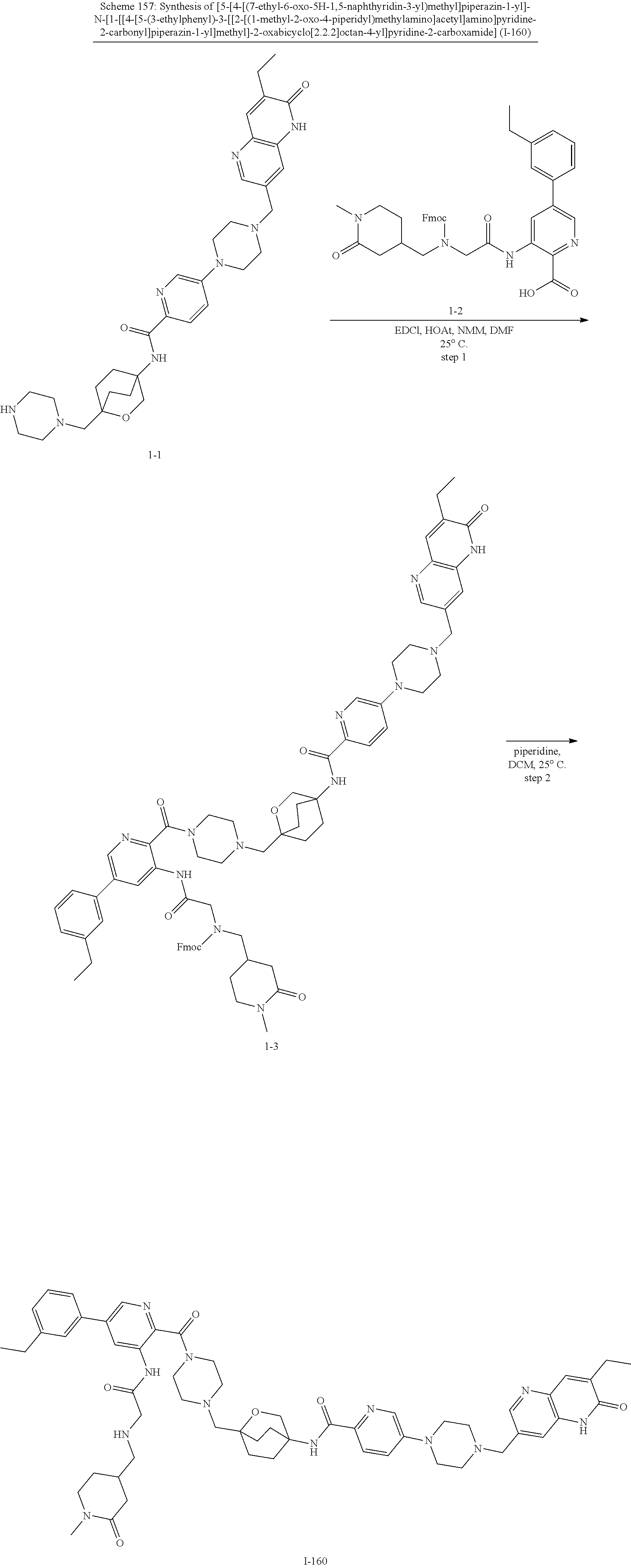

### Step 1: Synthesis of Intermediate 1-3

To a solution of Intermediate 1-2 (100 mg, 154.62 μmol, 1 eq) in DMF (1 mL) was added EDCI (89 mg, 463.87 μmol, 3 eq), HOAt (63 mg, 463.87 μmol, 3 eq), NMM (78 mg, 773.12 μmol, 85.00 μL, 5 eq) and intermediate 1-1 (111 mg, 185.55 μmol, 1.2 eq). The mixture was stirred at 25° C. for 2 hr. The reaction mixture was diluted with water (20 mL) and extracted with ethyl acetate (10 mL×3). The combined organic layers were washed with brine (20 mL), dried over anhydrous sodium sulfate, filtered and concentrated under reduced pressure to give intermediate 1-3 (100 mg, crude) as a yellow solid. LCMS (Method E): Retention time: 0.506 min, [M+H]⁺=1129.7.

### Step 2: Synthesis of I-160

To a solution of intermediate 1-3 (100 mg, 81.34 μmol, 1 eq) in DCM (1 mL) was added piperidine (69 mg, 813.36 μmol, 80.32 μL, 10 eq). The mixture was stirred at 25° C. for 1 hr. The reaction mixture was concentrated under reduced pressure to give the crude product. The residue was dissolved in DMF (2 mL) and purified by prep-HPLC (column: Phenomenex luna C18 150*25 mm*10 um; mobile phase: [water(FA)-ACN]; gradient: 5%-35% B over 9 min) and the organic phase was concentrated under reduced pressure to remove acetonitrile, and the liquid was lyophilized to give I-160 (3.87 mg, 3.77 μmol, 4.63% yield, 98% purity) as an off-white solid. LCMS (Method E): Retention time: 0.422 min, [M+H]⁺=1007.7. SFC: Retention time: 4.435 min. ¹H NMR (400 MHz, METHANOL-d4) δ=8.66-8.59 (m, 2H), 8.49 (d, J=1.6 Hz, 1H), 8.24 (d, J=2.8 Hz, 2H), 7.90-7.81 (m, 2H), 7.76 (s, 1H), 7.55-7.47 (m, 2H), 7.46-7.40 (m, 1H), 7.39-7.28 (m, 2H), 4.11 (s, 2H), 3.85 (s, 2H), 3.77-3.66 (m, 4H), 3.64-3.54 (m, 2H), 3.47-3.37 (m, 6H), 2.95 (s, 3H), 2.88-2.72 (m, 8H), 2.71-2.62 (m, 6H), 2.61-2.50 (m, 3H), 2.34-2.24 (m, 2H), 2.22-1.97 (m, 7H), 1.85-1.75 (m, 2H), 1.70-1.56 (m, 1H), 1.29 (d, J=2.4, 7.5 Hz, 6H).

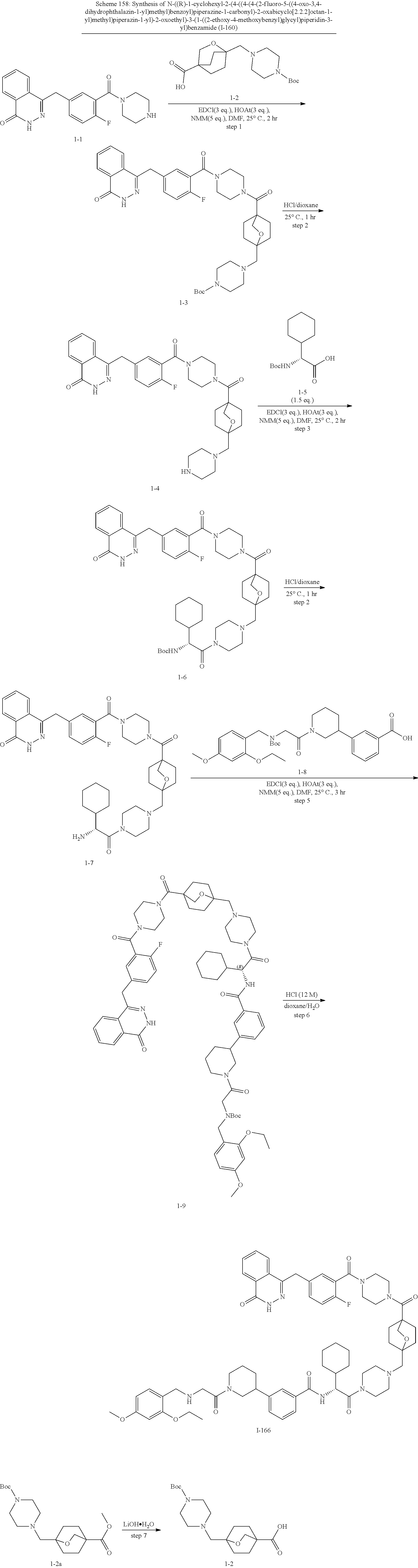

### Step 1: Synthesis of Intermediate 1-3

To a solution of Intermediate 1-1 (370 mg, 918.46 μmol, 1 eq, HCl salt) and Intermediate 1-2 (488.31 mg, 1.38 mmol, 1.5 eq) in DMF (4 mL) was added EDCI (528.21 mg, 2.76 mmol, 3 eq), NMM (464.51 mg, 4.59 mmol, 504.90 μL, 5 eq) and HOAt (375.03 mg, 2.76 mmol, 3 eq). The mixture was stirred at 25° C. for 2 hr. The reaction mixture was diluted with (30 mL) H₂O and filtered. The filter cake was used for purification. Intermediate 1-3 (540 mg, 729.92 μmol, 79.47% yield, 95% purity) was obtained as a yellow solid. LCMS (Method G): Rt=0.586 min, [M+H]⁺=703.5.

### Step 2: Synthesis of Intermediate 1-4

To a solution of Intermediate 1-3 (540 mg, 768.34 μmol, 1 eq) in dioxane (2 mL) was added HCl/dioxane (2 M, 3 mL, 7.81 eq). The mixture was stirred at 25° C. for 1 hr. The reaction mixture was concentrated under reduced pressure to give Intermediate 1-4 (490 mg, 732.13 μmol, 95.29% yield, 95.5% purity, HCl salt) was obtained as a yellow solid. LCMS (Method E): Rt=0.376 min, [M+H]⁺=603.3.

### Step 3: Synthesis of Intermediate 1-6

To a solution of Intermediate 1-4 (490 mg, 813.01 μmol, 1 eq) and Intermediate 1-5 (313.81 mg, 1.22 mmol, 1.5 eq) in DMF (5 mL) was added EDCI (467.57 mg, 2.44 mmol, 3 eq), NMM (411.17 mg, 4.07 mmol, 446.92 μL, 5 eq) and HOAt (331.98 mg, 2.44 mmol, 3 eq). The mixture was stirred at 25° C. for 2 hr. The reaction mixture was washed with water (50 mL) and extracted with DCM (30 mL*2). The combined organic phase was washed with brine (50 mL), dried with anhydrous Na₂SO₄, filtered and concentrated in vacuum to give a yellow oil. The crude product was purified by reversed-phase HPLC (0.1% NH₃·H₂O condition) to give Intermediate 1-6 (400 mg, 437.05 μmol, 53.76% yield, 92% purity) as an off-white solid. LCMS (Method G): Rt=0.659 min, [M+H]⁺=842.7. SFC: Rt=0.931 min.

### Step 4: Synthesis of Intermediate 1-7

To a solution of Intermediate 1-6 (400 mg, 475.06 μmol, 1 eq) in dioxane (1 mL) was added HCl/dioxane (2 M, 4 mL, 16.84 eq). The mixture was stirred at 25° C. for 1 hr. The reaction mixture was concentrated under reduced pressure to give an off-white solid. Intermediate 1-7 (350 mg, 346.24 mol, 72.89% yield, 77% purity, HCl salt) was obtained as an off-white solid. LCMS (Method G): Rt=0.563 min, [M+H]⁺=742.5.

### Step 5: Synthesis of Intermediate 1-9

To a solution of Intermediate 1-7 (150 mg, 192.71 μmol, 1 eq, HCl salt) and Intermediate 1-8 (152.23 mg, 289.07 μmol, 1.5 eq) in DMF (2 mL) was added EDCI (110.83 mg, 578.14 μmol, 3 eq), NMM (97.46 mg, 963.57 μmol, 105.94 μL, 5 eq) and HOAt (78.69 mg, 578.14 μmol, 80.88 μL, 3 eq). The mixture was stirred at 25° C. for 3 hr. The reaction mixture was washed with water (50 mL) and extracted with DCM (40 mL*2). The combined organic phase was washed with brine (50 mL*1), dried with anhydrous Na₂SO₄, filtered and concentrated in vacuum to give a yellow oil. The crude product was purified by reversed-phase HPLC (0.1% FA condition) to give Intermediate 1-9 (90 mg, 70.53 μmol, 36.60% yield, 98% purity) as an off-white solid. LCMS (Method G): Rt=0.763 min, [M+H]⁺=1250.9.

### Step 6: Synthesis of I-166

To a solution of Intermediate 1-9 (90 mg, 71.97 μmol, 1 eq) in dioxane (0.1 mL) and H₂O (1 mL) was added HCl (12 M, 12.00 μL, 2 eq). The mixture was stirred at 40° C. for 72 hr. The reaction mixture was diluted with 1 mL of MeOH and the mixture was used for purification. The crude product was purified by reversed-phase HPLC (0.1% FA condition) to give I-166 (6.49 mg, 5.54 μmol, 42.60% yield, 98.17% purity) as a brown solid. LCMS (Method E): Rt=0.476 min, [M+H]⁺=1150.7. SFC: Rt=5.242 min, 7.602 min. ¹H NMR (400 MHz, METHANOL-d₄) δ=8.37-8.10 (m, 1H), 7.98-7.93 (m, 1H), 7.91-7.80 (m, 2H), 7.77-7.68 (m, 2H), 7.51-7.41 (m, 3H), 7.36-7.30 (m, 1H), 7.24-7.13 (m, 2H), 6.59-6.47 (m, 2H), 4.64-4.44 (m, 2H), 4.38 (s, 2H), 4.13-4.05 (m, 2H), 3.96-3.90 (m, 4H), 3.83-3.64 (m, 14H), 3.58 (s, 3H), 3.21-3.10 (m, 1H), 2.80-2.68 (m, 2H), 2.61 (s, 2H), 2.56-2.48 (m, 2H), 2.30 (s, 2H), 2.19-2.10 (m, 2H), 2.05-1.75 (m, 12H), 1.68-1.55 (m, 4H), 1.43-1.40 (m, 4H), 1.34-1.23 (m, 3H), 1.16-1.04 (m, 2H).

### Step 7: Synthesis of Intermediate 1-2

To a solution of Intermediate 1-2a (600 mg, 1.63 mmol, 1 eq) in MeOH (2 mL), THF (2 mL) and H₂O (2 mL) was added LiOH·H₂O (683.32 mg, 16.28 mmol, 10 eq). The mixture was stirred at 25° C. for 2 hr. The reaction solution was acidified with HCl (1 M) to pH=5-6 and used for purification without workup. The crude product was purified by reversed-phase HPLC (0.1% HCl condition) to give a white solid. Intermediate 1-2 (500 mg, 1.37 mmol, 84.03% yield, 97% purity) was obtained as a white solid. LCMS (Method G): Rt=0.284 min, [M+H]⁺=355.2.

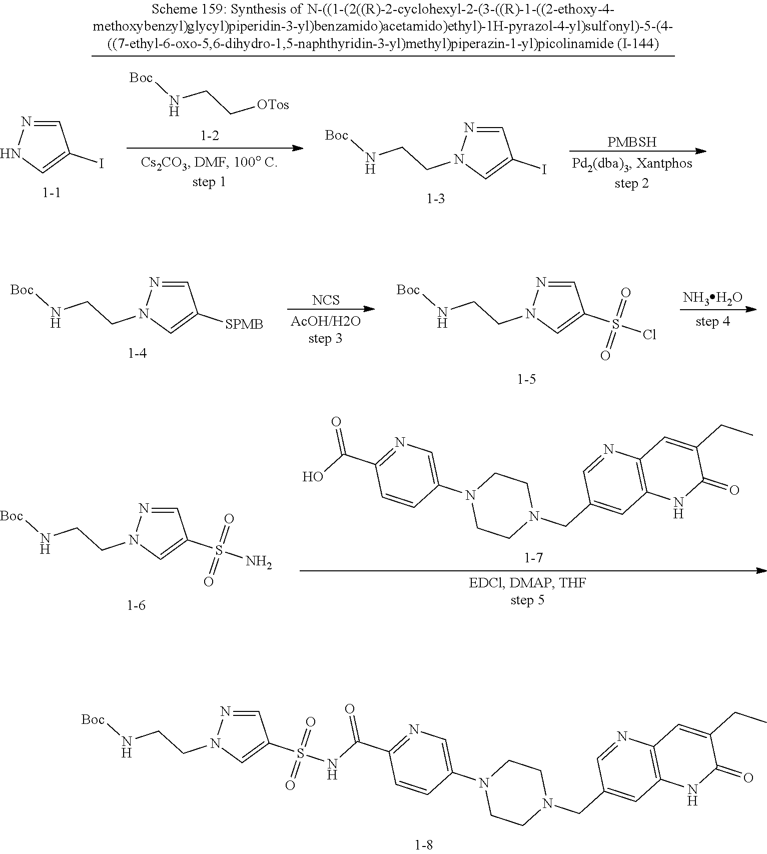

### Step 1: Synthesis of Intermediate 1-3

To a solution of Intermediate 1-1 (12 g, 61.86 mmol, 1 eq) in DMF (100 mL) was added Cs₂CO₃ (40.31 g, 123.73 mmol, 2 eq) and Intermediate 1-2 (25.36 g, 80.42 mmol, 1.3 eq). The mixture was stirred at 100° C. for 1 hr. The reaction mixture was poured into H₂O (200 mL) and white solid formed. The mixture was filtrated and the filter cake was concentrated to give Intermediate 1-3 (16 g, 47.46 mmol, 76.71% yield) as a white solid. LCMS (Method E): Rt=0.517 min, [M+H]⁺=338.2.

### Step 2: Synthesis of Intermediate 1-4

To a solution of Intermediate 1-3 (16 g, 47.46 mmol, 1 eq) in dioxane (190 mL) was added (4-methoxyphenyl)methanethiol (8.78 g, 56.95 mmol, 7.93 mL, 1.2 eq), Pd₂(dba)₃ (1.09 g, 1.19 mmol, 0.025 eq), Xantphos (1.37 g, 2.37 mmol, 0.05 eq) and DIEA (12.27 g, 94.91 mmol, 16.53 mL, 2 eq). The mixture was diluted with water (150 mL), extracted with EtOAc (150 mL*3) and washed with brine (30 mL*3). The organic layer was dried with anhydrous Na₂SO₄, filtered and concentrated under vacuum. The residue was purified by flash silica gel chromatography (ISCO; 220 g SepaFlash Silica Flash Column, Eluent of 0-56% Ethyl acetate/Petroleum ether gradient @100 mL/min) and concentrated under vacuum to give Intermediate 1-4 (15.7 g, 43.19 mmol, 91.02% yield) as yellow oil. LCMS (Method E): Rt=0.547 min, [M+H]⁺=364.1.

### Step 3: Synthesis of Intermediate 1-5

To a solution of Intermediate 1-4 (8.5 g, 23.39 mmol, 1 eq) in AcOH (64 mL) and H₂O (16 mL) was added NCS (12.49 g, 93.54 mmol, 4 eq) at 0° C. The mixture was stirred at 25° C. for 1 hr. The mixture was concentrated under vacuum and combined with another lot of product to give Intermediate 1-5 (1.6 g, 5.17 mmol, 22.09% yield) as yellow solid. LCMS (Method E): Rt=0.552 min, [M-Boc+H]⁺=209.9.

### Step 4: Synthesis of Intermediate 1-6

To a solution of Intermediate 1-5 (1.6 g, 5.17 mmol, 1 eq) in DCM (16 mL) was added NH₃·H₂O (3.64 g, 25.97 mmol, 4 mL, 25% purity, 5.03 eq). The mixture was stirred at 25° C. for 1.5 hr. The crude product was purified by reversed-phase HPLC (0.1% FA condition) and concentrated under vacuum to remove MeCN and dried by lyophilization to give Intermediate 1-6 (0.7 g, 2.41 mmol, 46.68% yield) as gray solid. LCMS (Method E): Rt=0.391 min, [M-Boc+H]⁺=191.2.

### Step 5: Synthesis of Intermediate 1-8

To a mixture of Intermediate 1-6 (0.5 g, 1.72 mmol, 1 eq), Intermediate 1-7 (745.31 mg, 1.89 mmol, 1.1 eq) and DMAP (631.16 mg, 5.17 mmol, 3 eq) in DMF (5 mL) was added EDCI (1.65 g, 8.61 mmol, 5 eq). The mixture was stirred at 40° C. for 2 hrs. The reaction was poured into MeOH (1 mL). The residue was purified by prep-HPLC (column: Phenomenex luna C18 150*40 mm*15 um; mobile phase: [water(FA)-ACN]; gradient: 8%-38% B over 15 min) and dried by lyophilization to give Intermediate 1-8 (490 mg, 736.00 μmol, 42.74% yield) as a yellow solid. LCMS: Rt=0.423 min, [M+H]⁺=666.7.

### Step 6: Synthesis of Intermediate 1-9

A mixture of Intermediate 1-8 (490 mg, 736.00 μmol, 1 eq) in DCM (5 mL) was added HCl/dioxane (2 M, 3 mL, 8.15 eq). The mixture was stirred at 25° C. for 5 min. The mixture was concentrated under vacuum to give Intermediate 1-9 (450 mg, crude, HCl salt) as yellow solid. LCMS (Method E): Rt=0.332 min, [M+H]⁺=566.3.

### Step 7: Synthesis of Intermediate 1-11

To a mixture of Intermediate 1-9 (450 mg, 747.38 μmol, 1 eq, HCl salt), Intermediate 1-10 (211.55 mg, 822.11 μmol, 1.1 eq) and HOAt (101.73 mg, 747.38 μmol, 1 eq) in DMF (5 mL) was added EDCI (286.54 mg, 1.49 mmol, 2 eq) and NMM (377.99 mg, 3.74 mmol, 410.85 μL, 5 eq). The mixture was stirred at 25° C. for 3 h hrs. The mixture was diluted with water (2 mL) and extracted with EtOAc (5 mL*3). The organic layer was concentrated under vacuum. The residue was purified by prep-HPLC (0.1% FA condition) and dried by lyophilization to give Intermediate 1-11 (0.1 g, 124.23 μmol, 16.62% yield) as white solid. LCMS (Method E): Rt=0.465 min, [M+H]⁺=805.5.

### Step 8: Synthesis of Intermediate 1-12

A mixture of Intermediate 1-11 (140 mg, 173.92 μmol, 1 eq) in DCM (1 mL) was added HCl/dioxane (2 M, 1 mL, 11.50 eq). The mixture was stirred at 25° C. for 1 hr. The mixture was concentrated under vacuum to give Intermediate 1-12 (130 mg, crude, HCl salt) as a yellow oil. LCMS (Method E): Rt=0.371 min, [M+H]⁺=705.4.

### Step 9: Synthesis of Intermediate 1-14

A mixture of Intermediate 1-12 (65 mg, 87.68 μmol, 1 eq, HCl salt), Intermediate 1-13 (46.18 mg, 87.68 μmol, 1 eq) and HOAt (11.93 mg, 87.68 μmol, 1 eq) in DMF (1 mL) was added EDCI (33.62 mg, 175.37 μmol, 2 eq) and NMM (44.35 mg, 438.42 μmol, 48.20 μL, 5 eq). The mixture was stirred at 25° C. for 1 hrs. The mixture was diluted with water (2 mL) and extracted with EtOAc (5 mL*3). The organic layer was concentrated under vacuum. The residue was purified by prep-HPLC (column: Phenomenex luna C18 150*25 mm*10 um; mobile phase: [water(FA)-ACN]; gradient: 35%-65% B over 9 min) and dried by lyophilization to give Intermediate 1-14 (55 mg, 45.33 μmol, 51.69% yield) as off-white solid. LCMS (Method E): Rt=0.542 min, [M+H]⁺=1213.6. SFC: Rt=1.783 min, 1.885 min.

### Step 10: Synthesis of I-144

A mixture of Intermediate 1-14 (55.00 mg, 45.33 μmol, 1 eq) in HCl (0.1 M, 1 mL, 2.21 eq) was stirred at 100° C. for 1 hr. The mixture was dried by lyophilization. The residue was purified by pre-HPLC (column: Phenomenex luna C18 150*25 mm*10 um; mobile phase: [water(FA)-ACN]; gradient: 18%-48% B over 9 min) and dried by lyophilization. 25 mg of reactant 1 was added in 0.1M HCl (0.6 mL) and stirred at 100° C. for 3 hrs. The residue was purified by Pre-HPLC (column: Phenomenex luna C18 150*25 mm*10 um; mobile phase: [water(FA)-ACN]; gradient: 15%-45% B over 9 min) and dried by lyophilization to get I-144 (20.33 mg, 18.26 μmol, 40.29% yield, 100% purity) as white solid. LCMS (Method G): Rt=0.448 min, [M+H]⁺=1113.7. SFC: Rt=2.634 min, 3.089 min. ¹H NMR (400 MHz, DMSO-d6) δ=12.00-11.67 (m, 1H), 8.43-8.22 (m, 2H), 8.20-8.14 (m, 1H), 8.13-7.94 (m, 2H), 7.87-7.80 (m, 1H), 7.80-7.38 (m, 7H), 7.33-7.17 (m, 2H), 6.57-6.39 (m, 2H), 4.49-4.35 (m, 1H), 4.33-4.10 (m, 4H), 4.10-3.85 (m, 6H), 3.77-3.62 (m, 9H), 3.23 (br s, 7H), 2.89-2.73 (m, 2H), 2.64 (br s, 1H), 2.54 (br d, J=7.2 Hz, 4H), 1.97-1.88 (m, 1H), 1.80-1.43 (m, 8H), 1.35-1.33 (m, 1H), 1.29-1.22 (m, 2H), 1.19-1.17 (m, 3H), 1.13-0.89 (m, 5H).

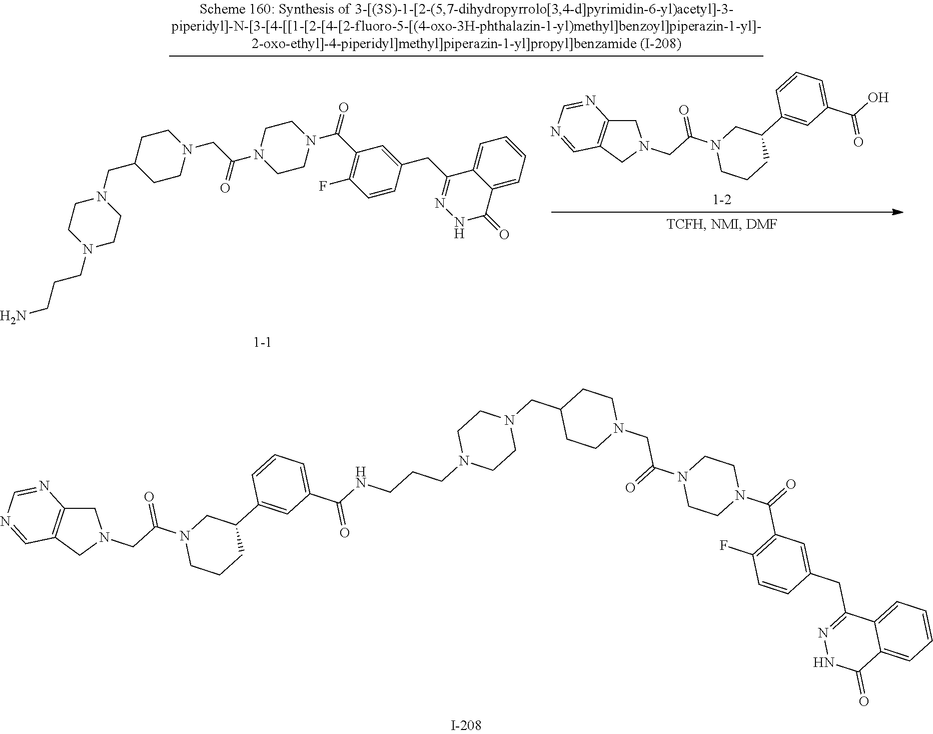

To a solution of Intermediate 1-1 (50 mg, 73.18 μmol, 1 eq, HCl salt) in DMF (0.5 mL) was added dropwise NMI (21.03 mg, 256.13 μmol, 20.42 μL, 3.5 eq) at 25° C., and then Intermediate 1-2 (32.18 mg, 87.81 μmol, 1.2 eq), TCFH (30.80 mg, 109.77 μmol, 1.5 eq) was added dropwise at 25° C. The resulting mixture was stirred at 25° C. for 1 hr. The reaction mixture was filtered, the filtrate was purified by prep-HPLC (column: Waters xbridge 150*25 mm 10 um; mobile phase: [water (NH₃H₂O)-ACN]; gradient: 17%-47% B over 14 min), the eluent was concentrated to remove ACN and lyophilized to give product (30 mg). The product was further purified by SFC (column: DAICEL CHIRALCEL OD (250 mm*30 mm, 10 um); mobile phase: [CO₂-ACN/EtOH (0.1% NH₃H₂O)]; B%: 62.5%, isocratic elution mode). The target peak was concentrated to afford I-208 (8.91 mg, 8.83 μmol, 31.98% yield, 98.68% purity) as yellow solid. LCMS (Method E): Rt=0.385 min, [M+H]⁺=996.8. SFC: Rt=1.396 min, ee %>99%. ¹H NMR (400 MHz, METHANOL-d₄) δ=8.98 (s, 1H), 8.63 (s, 1H), 8.36 (d, J=8.0 Hz, 1H), 7.98-7.91 (m, 1H), 7.90-7.80 (m, 2H), 7.75-7.64 (m, 2H), 7.50-7.34 (m, 4H), 7.18-7.16 (m, 1H), 4.77-4.54 (m, 2H), 4.38 (s, 2H), 4.22-4.12 (m, 4H), 4.04 (d, J=13.6 Hz, 1H), 3.90-3.82 (m, 1H), 3.81-3.65 (m, 5H), 3.58-3.48 (m, 2H), 3.45-3.40 (m, 2H), 3.35 (s, 1H), 3.26-3.11 (m, 3H), 2.95-2.81 (m, 3H), 2.80-2.67 (m, 2H), 2.47 (s, 9H), 2.19-2.18 (m, 2H), 2.09-2.01 (m, 3H), 1.91-1.81 (m, 4H), 1.78-1.65 (m, 3H), 1.64-1.56 (m, 1H), 1.24-1.12 (m, 2H).

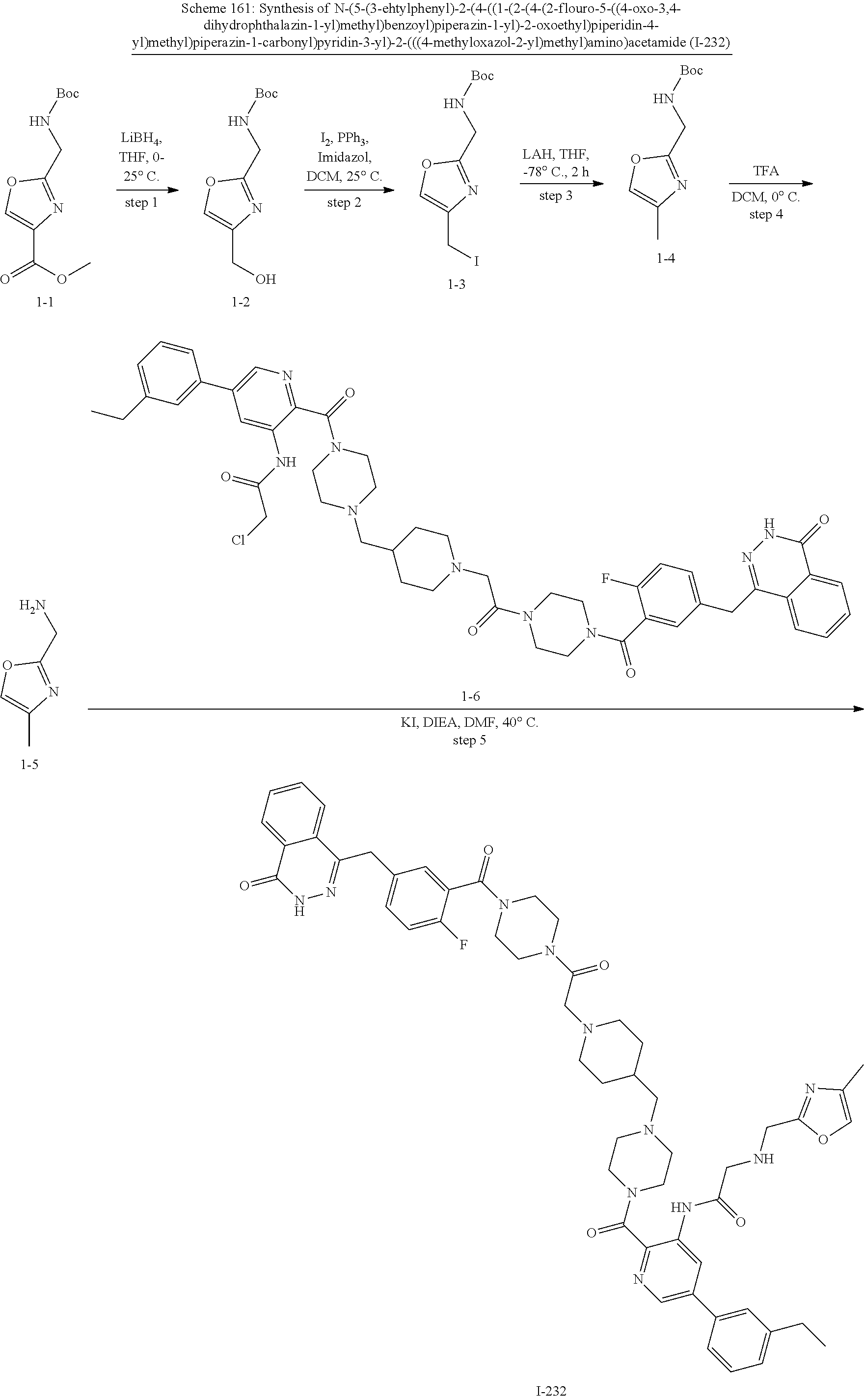

### Step 1: Synthesis of Intermediate 1-2

To a solution of Intermediate 1-1 (1 g, 3.90 mmol, 1 eq) in THF (20 mL) was added LiBH₄ (2 M, 7.80 mL, 4 eq) and EtOH (898.88 mg, 19.51 mmol, 5 eq) at 0° C. under N₂ and then the mixture was stirred at 25° C. for 1 h. The reaction mixture was quenched by MeOH (30 mL) and stirred at 25° C. for 0.5 h, then 1N HCl (5 mL) was added, then the mixture was stirred at 25° C. for 0.5, then H₂O (20 mL) was added, the mixture was extracted with DCM (30 mL*3), the combined organic layers was washed brine (30 mL*3), dried over anhydrous Na₂SO₄, filtered and the filtrate was concentrated under vacuum to give a residue. The residue was purified by flash silica gel chromatography (ISCO; 20 g SepaFlash Silica Flash Column, Eluent of 0˜10% DCM/MeOH @80 mL/min) and concentrated under vacuum to afford Intermediate 1-2 (740 mg, 3.24 mmol, 83.08% yield) as a yellow oil. LCMS (Method C): Rt=0.535 min, [M+H]⁺=229.2. ¹H NMR (400 MHz, CHLOROFORM-d) δ=7.53 (s, 1H), 5.56 (br s, 1H), 4.54 (s, 2H), 4.42 (br d, J=5.5 Hz, 2H), 1.44 (s, 9H).

### Step 2: Synthesis of Intermediate 1-3

To a solution of PPh₃ (896.33 mg, 3.42 mmol, 2 eq) in DCM (4 mL) was added I₂ (867.36 mg, 3.42 mmol, 688.38 μL, 2 eq). The mixture was stirred at 25° C. for 10 min, then imidazole (232.65 mg, 3.42 mmol, 2 eq) was added. The mixture was stirred at 25° C. for 10 min. Then a solution of Intermediate 1-2 (390 mg, 1.71 mmol, 1 eq) in DCM (4 mL) was added. The mixture was stirred at 25° C. for 1 h 40 min. The reaction mixture was quenched by sat. NaHCO₃ (2 mL) and extracted with DCM (2 mL*3), the combined organic layers were washed brine (2 mL*3), dried over anhydrous Na₂SO₄, filtered and the filtrate was concentrated under vacuum to give a residue. The residue was purified by flash silica gel chromatography (ISCO; 40 g SepaFlash Silica Flash Column, Eluent of 0˜30% PE/EA@120 mL/min) and concentrated under vacuum to afford Intermediate 1-3 (390 mg, 1.12 mmol, 65.75% yield, 97.4% purity) as a yellow oil. LCMS (Method G): Rt=0.540 min, [M−55]⁺=283.0. ¹H NMR (400 MHz, CHLOROFORM-d) δ=7.60 (s, 1H), 5.15 (br s, 1H), 4.43 (br d, J=5.5 Hz, 2H), 4.26 (s, 2H), 1.47 (s, 9H).

### Step 3: Synthesis of Intermediate 1-4

To a solution of Intermediate 1-3 (350 mg, 1.04 mmol, 1 eq) in THF (4 mL) was added LAH (2 M, 1.04 mL, 2 eq) under N₂. The mixture was stirred at −78° C. for 2 h. The reaction mixture was quenched by addition of Na₂SO₄·10H₂O (800 mg), stirred at 25° C. for 0. 5 h, filtered and concentrated under reduced pressure to give a residue. The residue was purified by flash silica gel chromatography (ISCO; 12 g SepaFlash Silica Flash Column, Eluent of 0˜20% Ethyl acetate/Petroleum ether gradient @18 mL/min) and concentrated under vacuum to afford Intermediate 1-4 (190 mg, 895.19 μmol, 86.49% yield) as yellow oil. ¹H NMR (400 MHz, CHLOROFORM-d) δ=7.31 (s, 1H), 5.21 (br s, 1H), 4.39 (br d, J=5.3 Hz, 2H), 2.14 (s, 3H), 1.45 (s, 9H).

### Step 4: Synthesis of Intermediate 1-5

To a solution of Intermediate 1-4 (100 mg, 471.15 μmol, 1 eq) in DCM (1 mL) was added TFA (0.2 mL) at 0° C. Then the mixture was stirred at 0° C. for 1 h. The mixture was concentrated under vacuum to give Intermediate 1-5 (80 mg, crude, TFA salt) as a light yellow solid. LCMS (Method G): Rt=0.120 min, [M+H]⁺=113.0.

### Step 5: Synthesis of I-232

To a solution of Intermediate 1-6 (80 mg, 89.84 μmol, 1 eq) in DMF (1.5 mL) was added DIEA (46.45 mg, 359.37 μmol, 62.60 μL, 4 eq), KI (59.66 mg, 359.37 μmol, 4 eq) and Intermediate 1-5 (30.48 mg, 134.76 μmol, 1.5 eq, TFA salt). The mixture was stirred at 40° C. for 1 h. The mixture was filtered. The filtrate was purified by prep-HPLC (column: Waters Xbridge 150*25 mm*5 um; mobile phase: [water (NH₄HCO₃)-ACN]; gradient: 30%-60% B over 15 min) and lyophilized to afford I-232 (32.11 mg, 32.66 μmol, 36.36% yield, 98.2% purity) as a white solid. LCMS (Method G): Rt=0.622 min, [M+H]⁺=966.6. ¹H NMR (400 MHz, METHANOL-d₄) δ=8.72 (d, J=1.6 Hz, 1H), 8.62 (d, J=2.0 Hz, 1H), 8.38 (m, 1H), 7.99-7.93 (m, 1H), 7.86 (m, 2H), 7.59 (d, J=0.8 Hz, 1H), 7.56-7.48 (m, 3H), 7.46-7.37 (m, 2H), 7.32 (d, J=7.6 Hz, 1H), 7.17 (m, 1H), 4.60 (br s, 1H), 4.39 (s, 2H), 3.97 (s, 2H), 3.84-3.67 (m, 6H), 3.58-3.44 (m, 6H), 3.31-3.23 (m, 2H), 2.94 (m, 2H), 2.76 (m, 2H), 2.59-2.45 (m, 4H), 2.26 (m, 2H), 2.17-2.06 (m, 5H), 1.79 (m, 2H), 1.65-1.54 (m, 1H), 1.36-1.18 (m, 6H). ¹⁹F NMR (377 MHz, METHANOL-d₄) δ =−120.7.

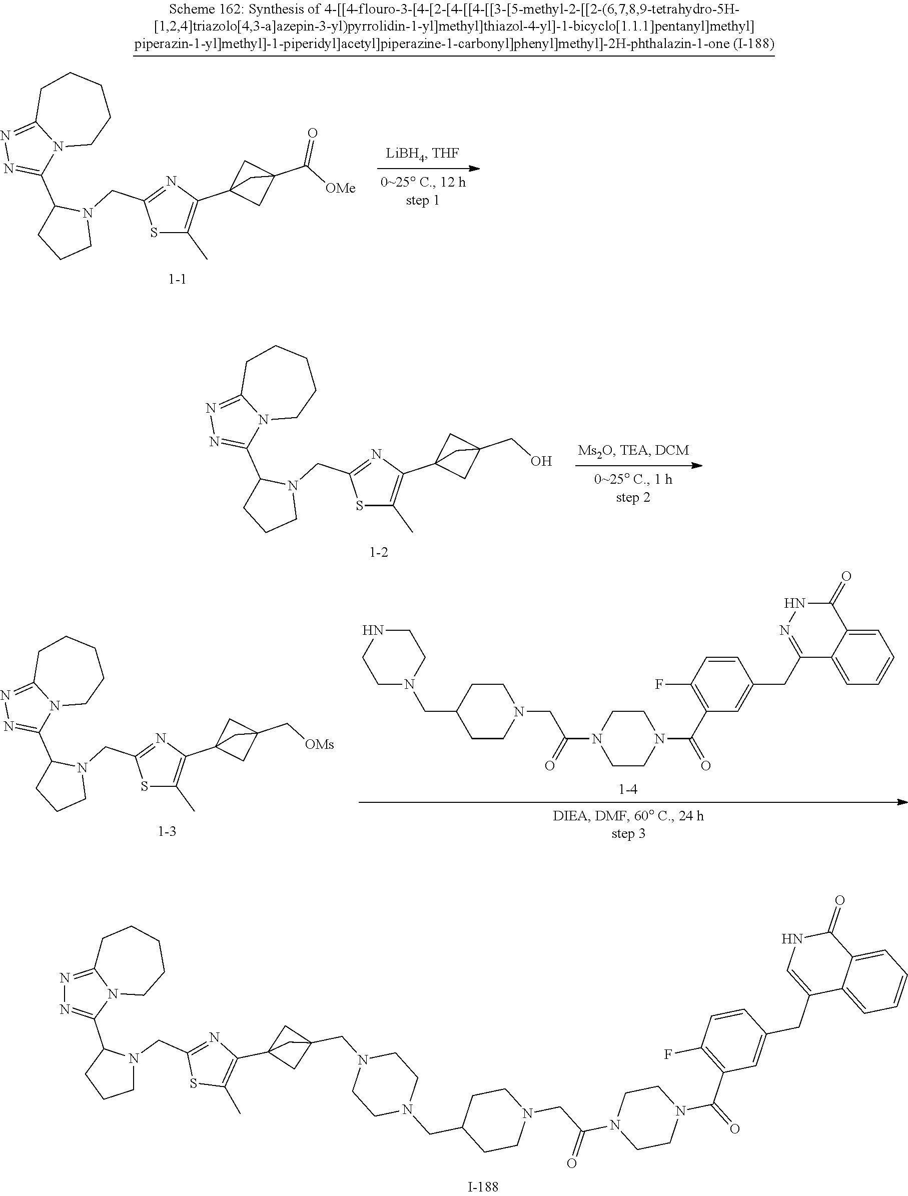 [SMILES: COC(=O)C12CC(c3nc(CN4CCCC4c4nnc5n4CCCCC5)sc3C)(C1)C2 ; Cc1sc(CN2CCCC2c2nnc3n2CCCCC3)nc1C12CC(CO)(C1)C2 ; Cc1sc(CN2CCCC2c2nnc3n2CCCCC3)nc1C12CC(COS(C)(=O)=O)(C1)C2 ; O=C(CN1CCC(CN2CCNCC2)CC1)N1CCN(C(=O)c2cc(Cc3n[nH]c(=O)c4ccccc34)ccc2F)CC1 ; Cc1sc(CN2CCCC2c2nnc3n2CCCCC3)nc1C12CC(CN3CCN(CC4CCN(CC(=O)N5CCN(C(=O)c6cc(Cc7c[nH]c(=O)c8ccccc78)ccc6F)CC5)CC4)CC3)(C1)C2]

### Step 1: Synthesis of Intermediate 1-2

Intermediate 1-1 (588 mg, 1.33 mmol, 1 eq) in THF (12 mL) was added LiBH₄ (2 M, 2.66 mL, 4 eq) at 0° C. and then the mixture was stirred at 25° C. for 12 hrs under N₂. The reaction mixture was quenched by MeOH (60 mL) and 2 N HCl (3 mL) and stirred at 60° C. for 4 hrs, then it was concentrated under vacuum to give a residue. The residue was purified by reversed-phase HPLC (0.10% FA condition) to afford Intermediate 1-2 (200 mg, 483.58 μmol, 36.32% yield) was obtained as a yellow solid. LCMS (Method E): Rt=0.390 min, [M+H]⁺=414.2. ¹H NMR (400 MHz, METHANOL-d₄): δ=5.13-5.03 (m, 1H), 4.77 (d, J=15.8 Hz, 1H), 4.57 (d, J=15.6 Hz, 1H), 4.41 (br d, J=4.6 Hz, 2H), 3.70-3.61 (m, 1H), 3.59 (s, 2H), 3.29-3.13 (m, 3H), 2.71-2.60 (m, 1H), 2.52 (s, 3H), 2.28-2.08 (m, 9H), 2.03-1.79 (m, 6H).

### Step 2: Synthesis of Intermediate 1-4

To a solution of Intermediate 1-2 (100 mg, 241.79 μmol, 1 eq) and TEA (73.40 mg, 725.38 μmol, 100.96 μL, 3 eq) in THF (2 mL) was added Ms₂O (63.18 mg, 362.69 μmol, 1.5 eq) at 0° C. Then the mixture was warmed to 25° C. and stirred at 25° C. for 1 h. The residue was poured into H₂O (4 mL). The aqueous phase was extracted with EA (3 mL*3). The combined organic phase was washed with brine (3 mL*3), dried with anhydrous sodium sulfate, filtered, and concentrated to give a residue. The residue was purified by flash silica gel chromatography (ISCO; 4 g SepaFlash Silica Flash Column, Eluent of 0˜100% Ethylacetate/Petroleum ether gradient @40 mL/min) and concentrated under vacuum to afford Intermediate 1-3 (120 mg, 244.07 μmol, 50.47% yield) as a yellow oil. LCMS (Method E): Rt=0.411 min, [M+H]⁺=492.2.

### Step 3: Synthesis of I-188

To a solution of Intermediate 1-3 (40 mg, 81.36 μmol, 1 eq) in DMF (1 mL) was added DIEA (52.57 mg, 406.78 μmol, 70.85 μL, 5 eq) and Intermediate 1-4 (47.98 mg, 81.36 μmol, 1 eq). The mixture was stirred at 60° C. for 24 hrs. The mixture was filtered. The filtrate was purified by Prep-HPLC (column: Phenomenex luna C18 150*25 mm*10 um; mobile phase: [water (FA)-ACN]; gradient: 2%-32% B over 9 min) to afford I-188 (11.17 mg, 11.18 μmol, 13.74% yield, 98.594% purity) as an orange solid. LCMS (Method E): Rt=0.400 min, [M+H]⁺=985.5. SFC: Rt=3.342 min, 4.726 min. ¹H NMR (400 MHz, METHANOL-d₄) δ=8.49 (s, 1H), 8.42-8.35 (m, 1H), 8.02-7.93 (m, 1H), 7.93-7.81 (m, 2H), 7.52 (m, 1H), 7.39 (m, 1H), 7.19 (m, 1H), 4.46-4.29 (m, 4H), 4.08-3.98 (m, 1H), 3.90-3.62 (m, 7H), 3.56-3.46 (m, 2H), 3.39-3.35 (m, 1H), 3.31-3.20 (m, 3H), 3.09-2.91 (m, 7H), 2.78 (br s, 4H), 2.70-2.36 (m, 9H), 2.35-2.20 (m, 7H), 2.18-1.65 (m, 13H), 1.52-1.28 (m, 3H). ¹⁹F NMR (377 MHz, METHANOL-d₄), δ=−120.7.

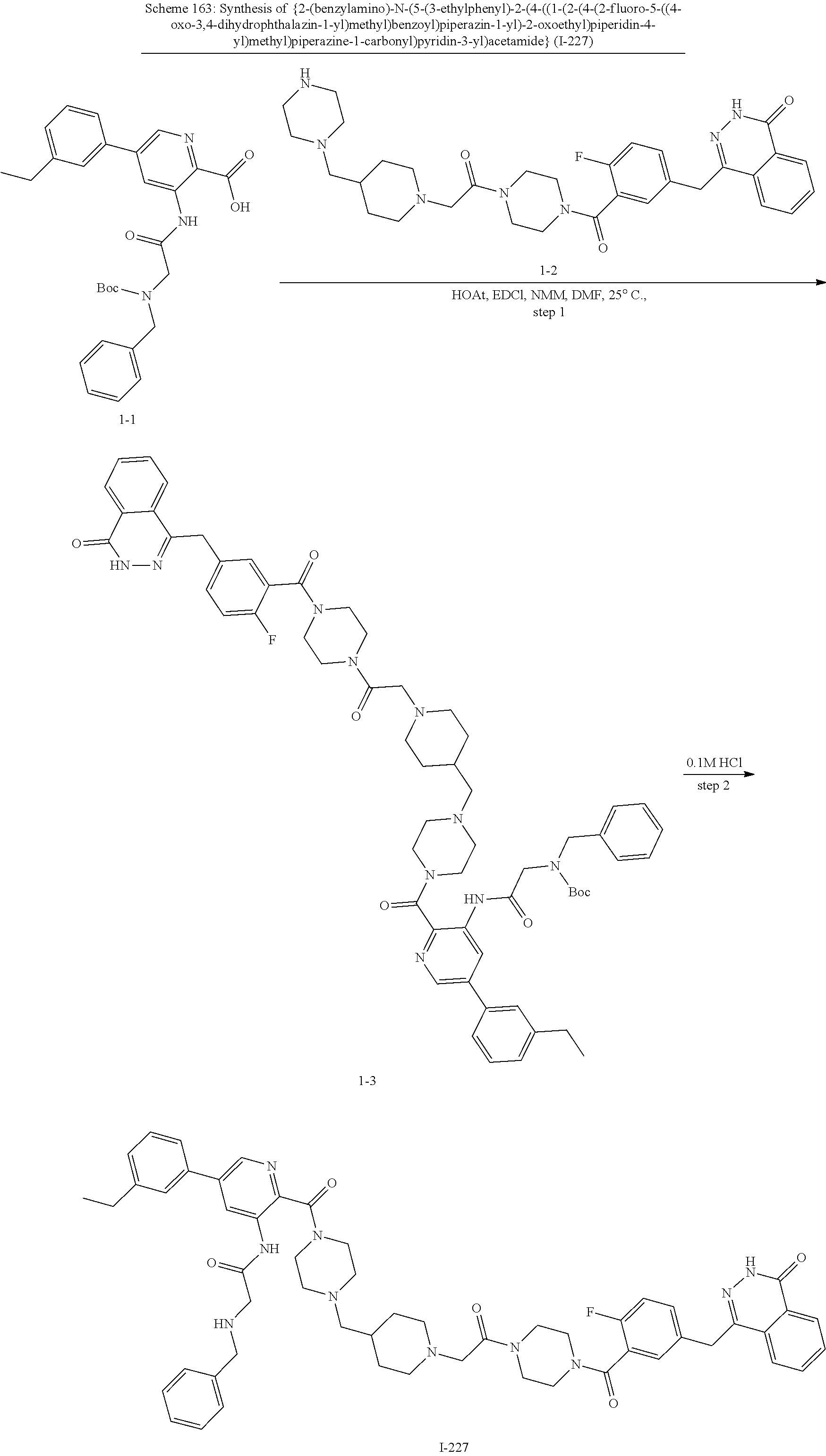 [SMILES: CCc1cccc(-c2cnc(C(=O)O)c(NC(=O)CN(Cc3ccccc3)C(=O)OC(C)(C)C)c2)c1 ; O=C(CN1CCC(CN2CCNCC2)CC1)N1CCN(C(=O)c2cc(Cc3n[nH]c(=O)c4ccccc34)ccc2F)CC1 ; CCc1cccc(-c2cnc(C(=O)N3CCN(CC4CCN(CC(=O)N5CCN(C(=O)c6cc(Cc7n[nH]c(=O)c8ccccc78)ccc6F)CC5)CC4)CC3)c(NC(=O)CN(Cc3ccccc3)C(=O)OC(C)(C)C)c2)c1 ; CCc1cccc(-c2cnc(C(=O)N3CCN(CC4CCN(CC(=O)N5CCN(C(=O)c6cc(Cc7n[nH]c(=O)c8ccccc78)ccc6F)CC5)CC4)CC3)c(NC(=O)CNCc3ccccc3)c2)c1]

### Step 1: Synthesis of Intermediate 1-3

To a solution of Intermediate 1-1 (50 mg, 102.13 μmol, 1 eq) in DMF (1 mL) was added HOAt (13.90 mg, 102.13 μmol, 14.29 μL, 1 eq), EDCI (39.16 mg, 204.26 μmol, 2 eq), NMM (51.65 mg, 510.66 μmol, 56.14 μL, 5 eq) and Intermediate 1-2 (63.95 mg, 102.13 μmol, 1 eq, HCl salt). The mixture was stirred at 25° C. for 1 h. The mixture was filtered, the filtrate was purified by reversed-phase HPLC (0.1% FA condition). The eluent was concentrated under reduced pressure to remove ACN, then lyophilized to afford Intermediate 1-3 (50 mg, 47.11 μmol, 46.13% yield) as a yellow solid. LCMS (Method E): Retention time: 0.483 min, (M+H)⁺=1061.6.

### Step 2: Synthesis of I-227

A solution of Intermediate 1-3 (50 mg, 47.11 μmol, 1 eq) in aq. HCl (0.1 M, 1 mL, 2.12 eq) was stirred at 100° C. for 2 h. To the mixture was added MeOH (0.5 mL). The mixture was purified by prep-HPLC (column: Phenomenex luna C18 150*25 mm*10 um; mobile phase: [water (FA)-ACN]; gradient: 5%-35% B over 9 min). The eluent was concentrated under reduced pressure to remove ACN, then lyophilized to afford I-227 (14.83 mg, 15.30 μmol, 32.47% yield, 99.1% purity) as an off-white solid. LCMS (Method G): Rt=0.701 min, (M+H)⁺=961.6. ¹H NMR (400 MHz, METHANOL-d4) δ=8.74 (d, J=2.0 Hz, 1H), 8.61 (d, J=2.0 Hz, 1H), 8.41-8.35 (m, 1H), 7.98-7.93 (m, 1H), 7.85 (m, 2H), 7.53-7.41 (m, 6H), 7.38-7.24 (m, 5H), 7.17 (m, 1H), 4.39 (s, 2H), 3.87-3.74 (m, 6H), 3.71-3.60 (m, 3H), 3.56-3.45 (m, 7H), 3.39-3.34 (m, 1H), 3.21-3.07 (m, 2H), 2.75 (m, 2H), 2.53-2.35 (m, 6H), 2.25 (m, 2H), 1.92-1.80 (m, 2H), 1.74-1.61 (m, 1H), 1.44-1.24 (m, 6H). ¹⁹F NMR (377 MHz, DMSO-d6) δ=−120.7.

### Scheme 164: Synthesis of 4-[[3-[4-[2-[2-[4-[[4-[3-[(3R)-1-[2-(5,7-dihydropyrrolo[3,4-d]pyrimidin-6-yl)acetyl]-3-piperidyl]benzoyl]piperazin-1-yl]methyl]-1-piperidyl]ethoxy]acetyl]piperazine-1-carbonyl]-4-fluoro-phenyl]methyl]-2H-phthalazin-1-one (I-226)

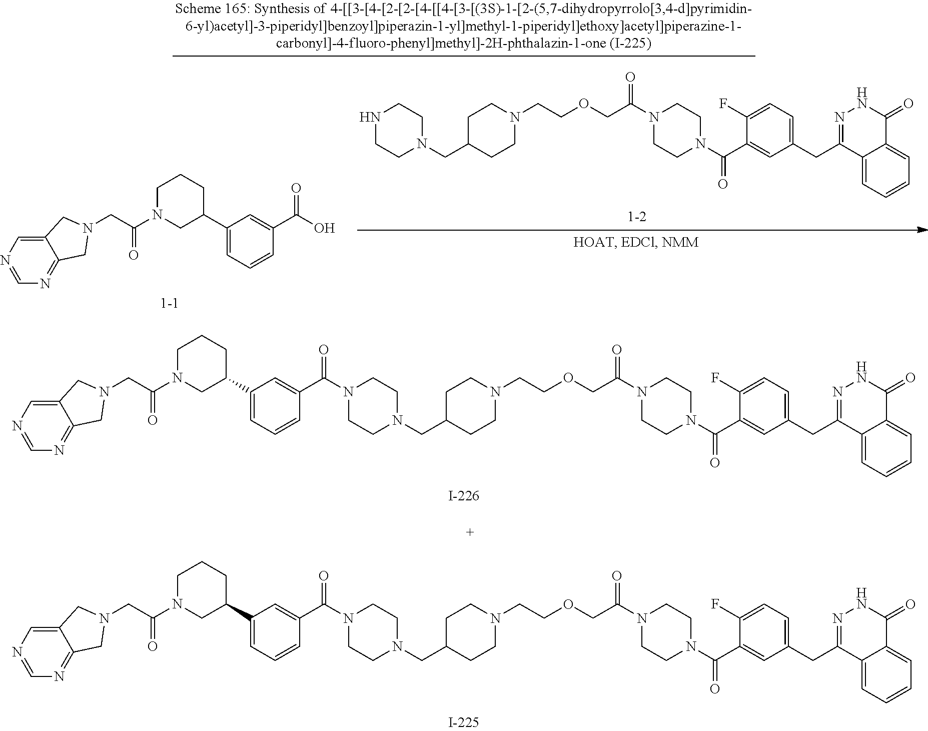 [SMILES: O=C(O)c1cccc(C2CCCN(C(=O)CN3Cc4cncnc4C3)C2)c1 ; O=C(COCCN1CCC(CN2CCNCC2)CC1)N1CCN(C(=O)c2cc(Cc3n[nH]c(=O)c4ccccc34)ccc2F)CC1 ; O=C(COCCN1CCC(CN2CCN(C(=O)c3cccc([C@H]4CCCN(C(=O)CN5Cc6cncnc6C5)C4)c3)CC2)CC1)N1CCN(C(=O)c2cc(Cc3n[nH]c(=O)c4ccccc34)ccc2F)CC1 ; O=C(COCCN1CCC(CN2CCN(C(=O)c3cccc([C@@H]4CCCN(C(=O)CN5Cc6cncnc6C5)C4)c3)CC2)CC1)N1CCN(C(=O)c2cc(Cc3n[nH]c(=O)c4ccccc34)ccc2F)CC1]

To a solution of Intermediate 1-1 (60 mg, 163.75 μmol, 1 eq) and Intermediate 1-2 (109.75 mg, 163.75 μmol, 1 eq, HCl salt) in DMF (0.6 mL) was added EDCI (94.17 mg, 491.25 μmol, 3 eq), HOAt (22.29 mg, 163.75 μmol, 22.91 μL, 1 eq) and NMM (165.63 mg, 1.64 mmol, 180.03 μL, 10 eq). The mixture was stirred at 25° C. for 1 hr. The mixture was poured into water (5 mL) and extracted with EA (5 mL*3). The combined organic layers were concentrated to give a residue. The residue was purified by prep-HPLC (column: YMC-Actus Triart C18 150*30 mm*7 um; mobile phase: [water (FA)-ACN]; gradient: 5%-35% B over 10 min) and the eluent was concentrated to remove MeCN and then lyophilized. The product was separated by SFC (column: DAICEL CHIRALCEL OD (250 mm*30 mm, 10 um); mobile phase: [CO₂-ACN/EtOH (0.1% NH₃H2O)]; B%: 60%, isocratic elution mode) and the eluent was concentrated to give I-226 (33.03 mg, 32.12 μmol, 19.62% yield, FA salt) as a yellow solid and I-225 (27.18 mg, 26.44 μmol, 16.14% yield, FA) as a yellow solid LCMS (Method E): Rt=0.393 min, [M+H]⁺=982.5.

LCMS (Method E): Rt=0.375 min, [M+H]⁺=982.5.

LCMS (Method E): Rt=0.375 min, [M+H]⁺=982.5.

SFC: Rt=1.144 min, ee value >99%.

SFC: Rt=1.885 min, ee value=99.7%.

¹H NMR (400 MHz, METHANOL-d₄) δ=8.98 (s, 1H), 8.66-8.61 (m, 1H), 8.48 (br s, 1H), 8.40-8.33 (m, 1H), 7.99-7.92 (m, 1H), 7.91-7.79 (m, 2H), 7.50 (br s, 1H), 7.45-7.23 (m, 5H), 7.17-7.10 (m, 1H), 4.61-4.52 (m, 1H), 4.48-4.34 (m, 4H), 4.25-4.10 (m, 4H), 4.08-3.99 (m, 1H), 3.93-3.39 (m, 15H), 3.38-3.32 (m, 4H), 3.23-2.97 (m, 3H), 2.89-2.68 (m, 2H), 2.59-2.26 (m, 6H), 2.07 (d, J=12.8 Hz, 3H), 1.98-1.78 (m, 3H), 1.73-1.46 (m, 3H), 1.37-1.26 (m, 1H)

¹H NMR (400 MHz, METHANOL-d₄) δ=9.00-8.95 (m, 1H), 8.63 (s, 1H), 8.55 (s, 1H), 8.36 (d, J=8.0 Hz, 1H), 7.94 (d, J=3.2 Hz, 1H), 7.91-7.80 (m, 2H), 7.52-7.46 (m, 1H), 7.44-7.23 (m, 5H), 7.16-7.10 (m, 1H), 4.60-4.53 (m, 1H), 4.38 (s, 2H), 4.30-4.10 (m, 6H), 4.07-3.98 (m, 1H), 3.91-3.57 (m, 10H), 3.55-3.39 (m, 4H), 3.22-3.12 (m, 1H), 3.09-2.96 (m, 2H), 2.88-2.61 (m, 4H), 2.50 (br s, 2H), 2.36 (d, J=11.2 Hz, 2H), 2.27-2.09 (m, 5H), 2.03-2.00 (m, 1H), 1.92-1.73 (m, 4H), 1.70-1.54 (m, 2H), 1.37-1.15 (m, 3H)

¹⁹F NMR (377 MHz, METHANOL-d₄) δ=−120.7.

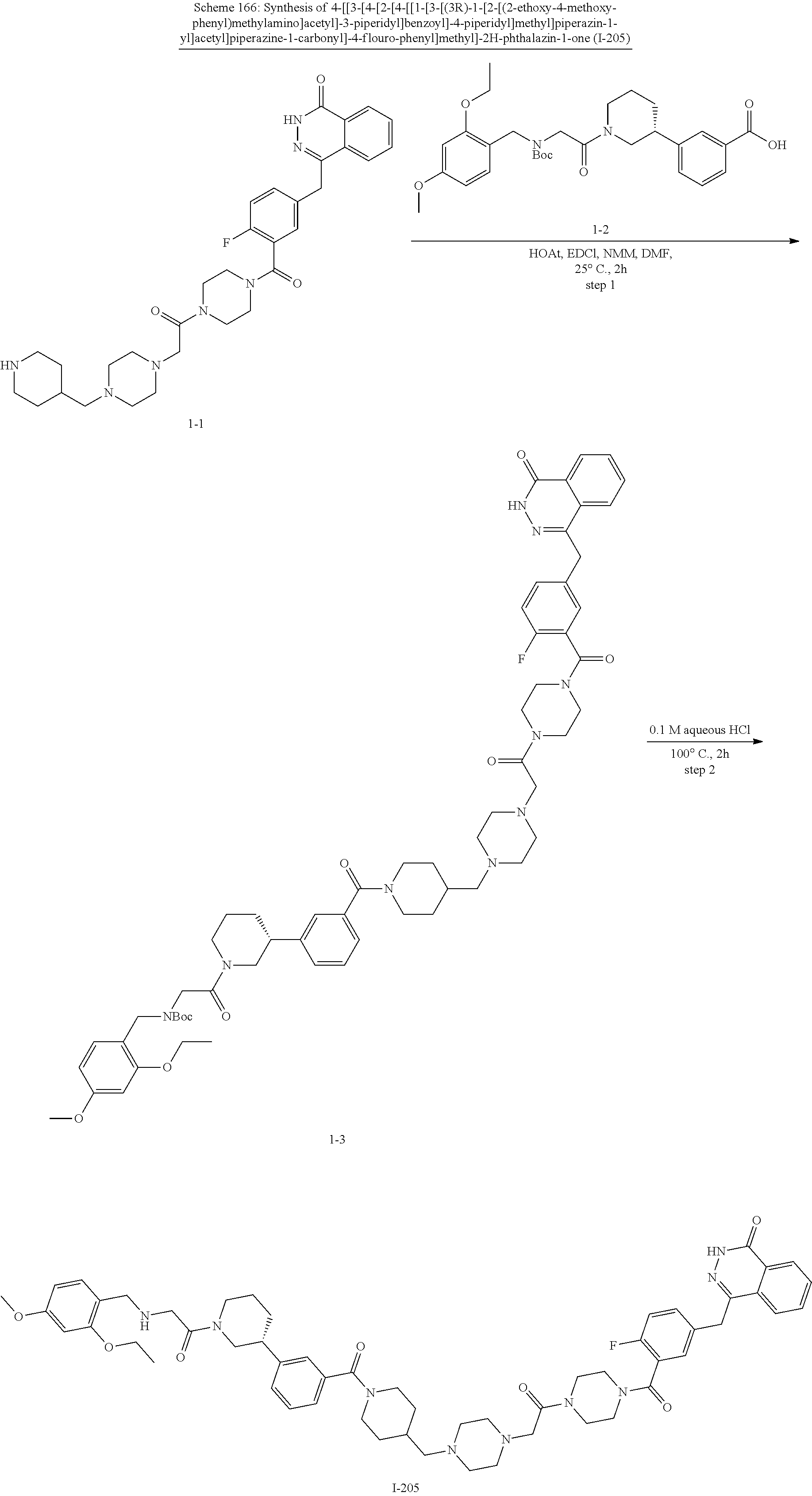 [SMILES: O=C(CN1CCN(CC2CCNCC2)CC1)N1CCN(C(=O)c2cc(Cc3n[nH]c(=O)c4ccccc34)ccc2F)CC1 ; CCOc1cc(OC)ccc1CN(CC(=O)N1CCC[C@H](c2cccc(C(=O)O)c2)C1)C(=O)OC(C)(C)C ; CCOc1cc(OC)ccc1CN(CC(=O)N1CCC[C@H](c2cccc(C(=O)N3CCC(CN4CCN(CC(=O)N5CCN(C(=O)c6cc(Cc7n[nH]c(=O)c8ccccc78)ccc6F)CC5)CC4)CC3)c2)C1)C(=O)OC(C)(C)C ; CCOc1cc(OC)ccc1CNCC(=O)N1CCC[C@H](c2cccc(C(=O)N3CCC(CN4CCN(CC(=O)N5CCN(C(=O)c6cc(Cc7n[nH]c(=O)c8ccccc78)ccc6F)CC5)CC4)CC3)c2)C1]

### Step 1: Synthesis of Intermediate 1-3

A mixture of Intermediate 1-1 (50 mg, 79.85 μmol, 1 eq, HCl salt), Intermediate 1-2 (42.05 mg, 79.85 μmol, 1 eq) and HOAt (10.87 mg, 79.85 μmol, 11.17 μL, 1 eq) in DMF (1 mL) was added EDCI (30.62 mg, 159.70 μmol, 2 eq) and NMM (40.38 mg, 399.26 μmol, 43.90 μL, 5 eq). The mixture was stirred at 25° C. for 2 hrs. The mixture was poured into water (1 mL). The residue was purified by prep-HPLC (column: Waters Xbridge 150*25 mm*5 um; mobile phase: [water (NH₄HCO₃)-ACN]; gradient: 42%-72% B over 9 min) and dried by lyophilization to afford Intermediate 1-3 (60 mg, 54.63 μmol, 68.41% yield) as a white solid. LCMS (Method G): Rt=0.678 min, [M+H]⁺=1098.8.

### Step 2: Synthesis of I-205

A mixture of Intermediate 1-3 (0.06 g, 54.63 μmol, 1 eq) and aq. HCl (0.1 M, 1.64 mL, 3 eq) was stirred at 100° C. for 2 hrs. The mixture was purified by prep-HPLC (column: Phenomenex Luna C18 150*25 mm*10 um; mobile phase: [water (HCl)-ACN]; gradient: 8%-38% B over 10 min). The eluent was lyophilized to give I-205 (20.02 mg, 18.78 μmol, 75.0% yield, 97.0% purity, HCl salt) as a white solid. LCMS (Method G): Rt=0.632 min, [M+H]⁺=998.6. SFC: Rt=1.557 min. ¹H NMR (400 MHz, DMSO-d₆) δ=12.60 (br s, 1H), 9.18-8.77 (m, 2H), 8.26 (d, J=7.6 Hz, 1H), 8.03-7.79 (m, 3H), 7.50-7.31 (m, 6H), 7.28-7.22 (m, 2H), 6.65-6.52 (m, 2H), 4.52-4.38 (m, 2H), 4.37-4.32 (m, 2H), 4.14-4.04 (m, 5H), 4.03-3.91 (m, 2H), 3.77 (d, J=3.6 Hz, 4H), 3.72-3.55 (m, 8H), 3.53-3.46 (m, 4H), 3.30-3.17 (m, 5H), 3.16-2.99 (m, 4H), 2.87-2.74 (m, 2H), 2.71-2.53 (m, 2H), 2.21-2.06 (m, 1H), 2.01-1.72 (m, 5H), 1.44-1.29 (m, 4H), 1.27-1.07 (m, 2H). ¹⁹F NMR (377 MHz, DMSO-d6) δ=−119.7.

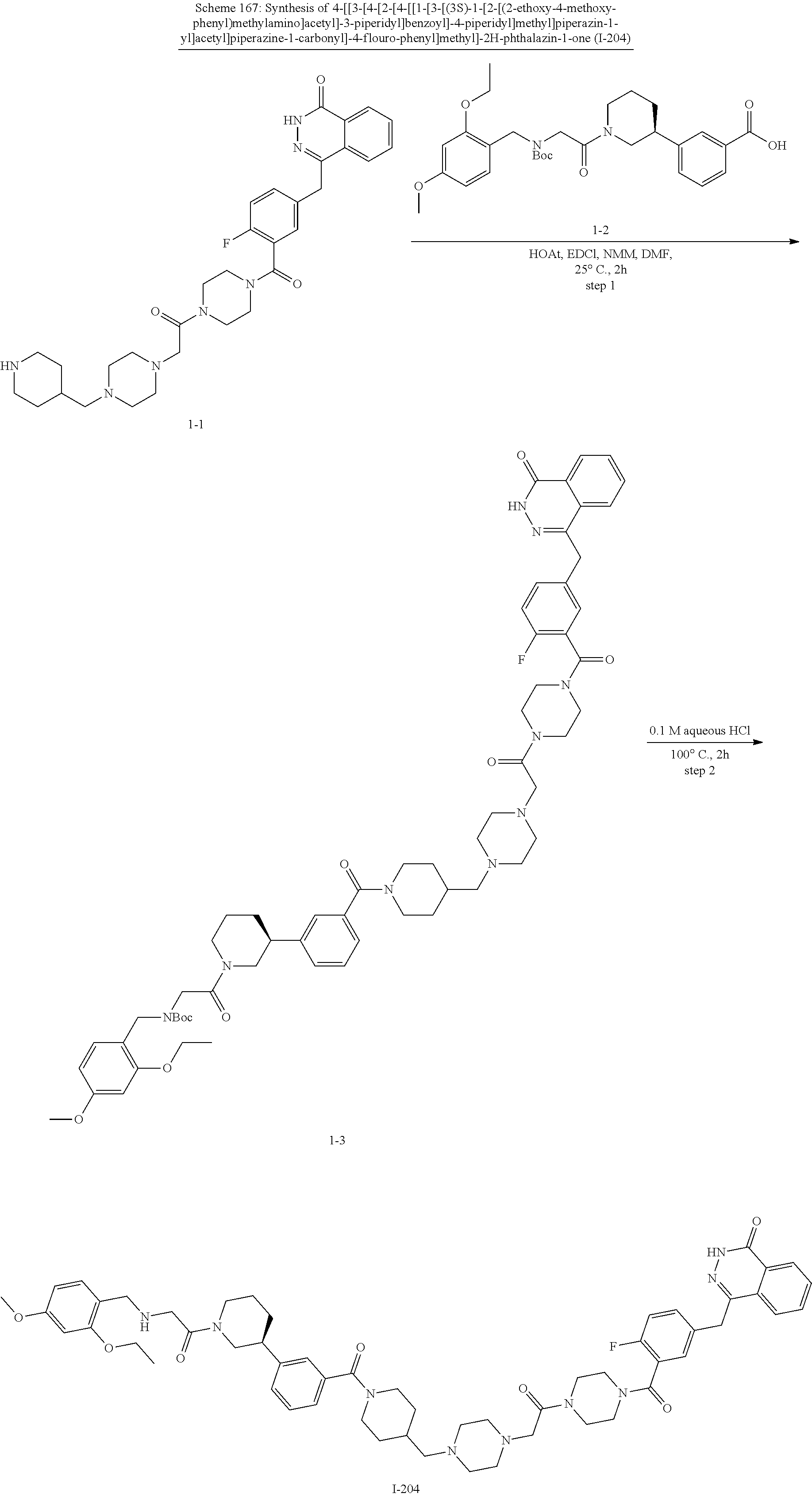 [SMILES: O=C(CN1CCN(CC2CCNCC2)CC1)N1CCN(C(=O)c2cc(Cc3n[nH]c(=O)c4ccccc34)ccc2F)CC1 ; CCOc1cc(OC)ccc1CN(CC(=O)N1CCC[C@@H](c2cccc(C(=O)O)c2)C1)C(=O)OC(C)(C)C ; CCOc1cc(OC)ccc1CN(CC(=O)N1CCC[C@@H](c2cccc(C(=O)N3CCC(CN4CCN(CC(=O)N5CCN(C(=O)c6cc(Cc7n[nH]c(=O)c8ccccc78)ccc6F)CC5)CC4)CC3)c2)C1)C(=O)OC(C)(C)C ; CCOc1cc(OC)ccc1CNCC(=O)N1CCC[C@@H](c2cccc(C(=O)N3CCC(CN4CCN(CC(=O)N5CCN(C(=O)c6cc(Cc7n[nH]c(=O)c8ccccc78)ccc6F)CC5)CC4)CC3)c2)C1]

### Step 1: Synthesis of Intermediate 1-3

A mixture of Intermediate 1-1 (50 mg, 79.85 μmol, 1 eq, HCl salt), Intermediate 1-2 (42.05 mg, 79.85 μmol, 1 eq) and HOAt (10.87 mg, 79.85 μmol, 11.17 μL, 1 eq) in DMF (1 mL) was added EDCI (30.62 mg, 159.70 μmol, 2 eq) and NMM (40.38 mg, 399.26 μmol, 43.90 μL, 5 eq). The mixture was stirred at 25° C. for 2 hrs. The mixture was poured into water (1 mL). The residue was purified by prep-HPLC (column: Waters Xbridge 150*25 mm*5 um; mobile phase: [water (NH₄HCO₃)-ACN]; gradient: 42%-72% B over 9 min) and dried by lyophilization to afford Intermediate 1-3 (60 mg, 54.63 μmol, 68.41% yield) as a white solid. LCMS (Method G): Rt=0.680 min, [M+H]⁺=1098.8.

### Step 2: Synthesis of I-204

A mixture of Intermediate 1-3 (55 mg, 50.08 μmol, 1 eq) and aq. HCl (0.1 M, 1.50 mL, 3 eq) was stirred at 100° C. for 2 hrs. The mixture was purified by prep-HPLC (column: Phenomenex Luna C18 150*25 mm*10 um; mobile phase: [water (HCl)-ACN]; gradient: 8%-38% B over 10 min). The eluent was lyophilized to give I-204 (16.95 mg, 15.56 μmol, 77.67% yield, 94.9% purity, HCl salt) as a white solid. LCMS (Method G): Rt=0.624 min, [M+H]⁺=998.6. SFC: Rt=1.445 min. ¹H NMR (400 MHz, DMSO-d₆) δ=12.61 (d, J=2.8 Hz, 1H), 9.07-8.85 (m, 2H), 8.26 (d, J=7.6 Hz, 1H), 8.02-7.79 (m, 3H), 7.50-7.44 (m, 1H), 7.44-7.32 (m, 5H), 7.28-7.22 (m, 2H), 6.65-6.51 (m, 2H), 4.50-4.36 (m, 2H), 4.34 (s, 2H), 4.13-4.03 (m, 5H), 4.03-3.90 (m, 2H), 3.77 (d, J=3.6 Hz, 4H), 3.71-3.54 (m, 8H), 3.48 (br d, J=3.6 Hz, 5H), 3.28-3.17 (m, 4H), 3.16-2.98 (m, 4H), 2.85-2.74 (m, 2H), 2.69-2.58 (m, 1H), 2.18-2.03 (m, 1H), 2.00-1.88 (m, 2H), 1.86-1.69 (m, 3H), 1.64-1.43 (m, 1H), 1.42-1.27 (m, 4H), 1.26-1.09 (m, 2H). ¹⁹F NMR (377 MHz, DMSO-d6) δ=−119.7.

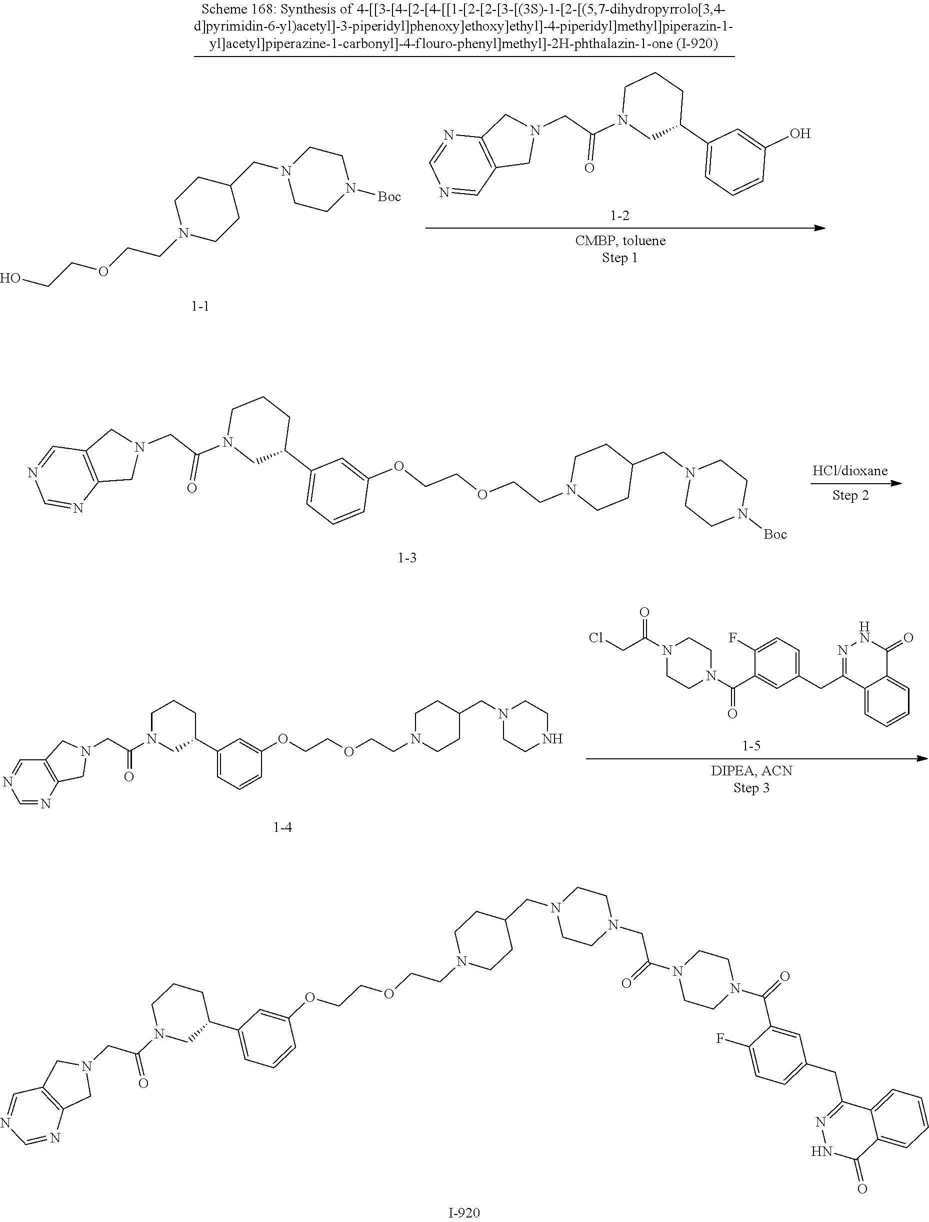 [SMILES: CC(C)(C)OC(=O)N1CCN(CC2CCN(CCOCCO)CC2)CC1 ; O=C(CN1Cc2cncnc2C1)N1CCC[C@H](c2cccc(O)c2)C1 ; CC(C)(C)OC(=O)N1CCN(CC2CCN(CCOCCOc3cccc([C@H]4CCCN(C(=O)CN5Cc6cncnc6C5)C4)c3)CC2)CC1 ; O=C(CN1Cc2cncnc2C1)N1CCC[C@H](c2cccc(OCCOCCN3CCC(CN4CCNCC4)CC3)c2)C1 ; O=C(CCl)N1CCN(C(=O)c2cc(Cc3n[nH]c(=O)c4ccccc34)ccc2F)CC1 ; O=C(CN1CCN(CC2CCN(CCOCCOc3cccc([C@H]4CCCN(C(=O)CN5Cc6cncnc6C5)C4)c3)CC2)CC1)N1CCN(C(=O)c2cc(Cc3n[nH]c(=O)c4ccccc34)ccc2F)CC1]

### Step 1: Synthesis of Intermediate 1-3

To a mixture of Intermediate 1-1 (131.74 mg, 354.61 μmol, 1.2 eq) and Intermediate 1-2 (100.00 mg, 295.51 μmol, 1 eq) in toluene (1 mL) was added 2-(tributyl-phosphanylidene)acetonitrile (213.96 mg, 886.52 μmol, 3 eq), then the mixture was stirred at 90° C. for 1 hr under N₂. The reaction mixture was concentrated under reduced pressure to give a residue. The residue was purified by flash silica gel chromatography ((ISCO; 12 g SepaFlash Silica Flash Column, Eluent of 0˜80% Ethyl acetate/Petroleum ether gradient @60 mL/min).). The eluent was concentrated under reduced pressure to give Intermediate 1-3 (90 mg, 121.62 μmol, 41.16% yield, 93.5% purity) as a white solid. LCMS (Method D): Rt=0.255 min, [M+H]⁺=692.7.

### Step 2: Synthesis of Intermediate 1-4

A mixture of Intermediate 1-3 (80 mg, 115.62 μmol, 1 eq) in HCl/dioxane (1 mL) was stirred at 20° C. for 10 min. The reaction mixture was concentrated under reduced pressure to give Intermediate 1-4 (100 mg, crude, HCl salt) was obtained as a white solid. LCMS (Method D): Rt=0.195 min, [M+H]⁺=592.5.

### Step 3: Synthesis of I-920

To a mixture of Intermediate 1-4 (70 mg, 111.42 μmol, 1 eq, HCl salt) and Intermediate 1-5 (49.35 mg, 111.42 μmol, 1 eq) in ACN (1 mL) was added DIEA (43.20 mg, 334.26 μmol, 58.22 μL, 3 eq), then the mixture was 60° C. for 1 hr. The reaction mixture was concentrated under reduced pressure to give a residue. The crude product was purified by reversed-phase HPLC (0.1% FA condition). The eluent was concentrated under reduced pressure to remove MeCN and then lyophilized to give I-920 (19 mg, 18.92 μmol, 16.98% yield, 99.4% purity) as a yellow gum. LCMS (Method D): Rt=0.237 min, [M+H]⁺=998.4. SFC: Retention time: 6.820 min. ¹H NMR (400 MHz, METHANOL-d4) δ=8.98 (d, J=5.6 Hz, 1H), 8.64 (d, J=4.4 Hz, 1H), 8.54-8.40 (m, 1H), 8.37 (d, J=7.6 Hz, 1H), 7.99-7.91 (m, 1H), 7.91-7.78 (m, 2H), 7.54-7.46 (m, 1H), 7.39-7.34 (m, 1H), 7.29-7.11 (m, 2H), 6.93-6.72 (m, 3H), 4.60-4.49 (m, 1H), 4.39 (s, 2H), 4.25-4.08 (m, 6H), 4.02 (d, J=14.4 Hz, 1H), 3.94-3.65 (m, 10H), 3.60-3.48 (m, 4H), 3.38-3.31 (m, 6H), 3.18-3.10 (m, 1H), 3.05-2.92 (m, 2H), 2.80-2.46 (m, 10H), 2.30-2.26 (m, 2H), 2.07-1.93 (m, 3H), 1.91-1.77 (m, 3H), 1.73-1.58 (m, 1H), 1.50-1.35 (m, 2H). ¹⁹F NMR (376 MHz, METHANOL-d4) δ=−120.81.

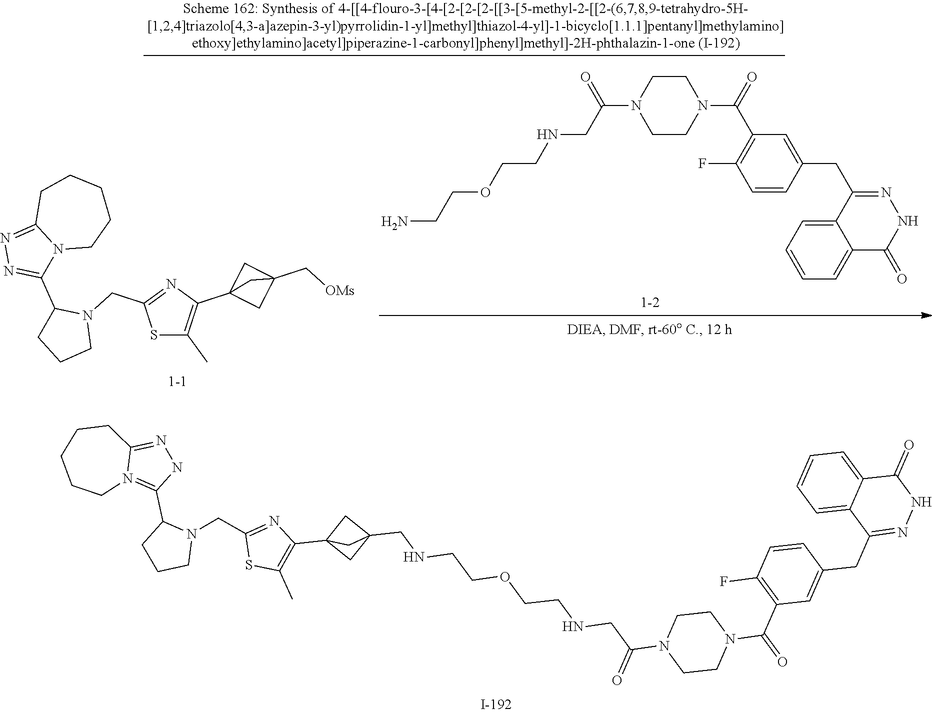

To a solution of Intermediate 1-1 (30 mg, 61.02 μmol, 1 eq) in DMF (0.8 mL) was added DIEA (39.43 mg, 305.08 μmol, 53.14 μL, 5 eq) and Intermediate 1-2 (31.15 mg, 61.02 μmol, 1 eq). The mixture was stirred at 60° C. for 12 hrs. The mixture was filtered and the filtrate was purified by prep-HPLC (column: Phenomenex luna C18 150*25 mm*10 um; mobile phase: [water (FA)-ACN]; gradient: 5%-35% B over 9 min), and the eluent was concentrated under reduced pressure to remove ACN, then lyophilized to afford I-192 (5.82 mg, 6.28 μmol, 10.30% yield, 97.8% purity) as a white solid. LCMS (Method E): Rt=0.395 min, [M+H]⁺=906.4. SFC: Rt=0.872 min, 1.536 min. ¹H NMR (400 MHz, METHANOL-d₄) δ=8.42 (s, 2H), 8.37 (m, 1H), 7.99-7.92 (m, 1H), 7.91-7.81 (m, 2H), 7.55-7.47 (m, 1H), 7.40-7.32 (m, 1H), 7.17 (m, 1H), 4.39 (s, 4H), 4.04-3.92 (m, 2H), 3.91-3.67 (m, 10H), 3.64-3.50 (m, 3H), 3.38 (br s, 2H), 3.30-3.23 (m, 4H), 3.15-3.04 (m, 2H), 2.98-2.88 (m, 2H), 2.49-2.36 (m, 4H), 2.35-2.22 (m, 6H), 2.21-2.09 (m, 1H), 2.08-1.86 (m, 5H), 1.85-1.59 (m, 4H), 1.40-1.29 (m, 1H). ¹⁹FNMR (377 MHz, METHANOL-d₄) δ=−120.7.

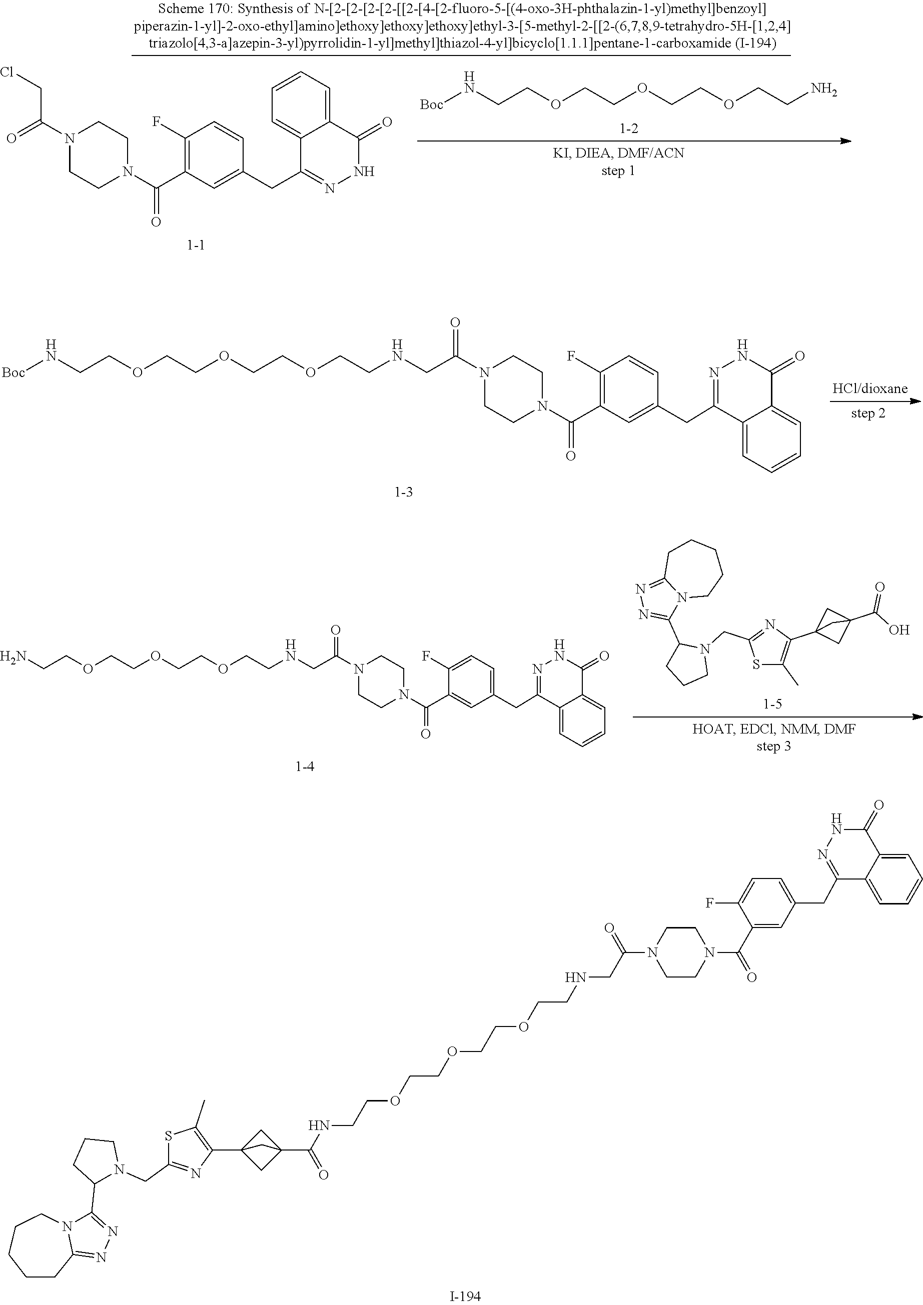 [SMILES: O=C(CCl)N1CCN(C(=O)c2cc(Cc3n[nH]c(=O)c4ccccc34)ccc2F)CC1 ; CC(C)(C)OC(=O)NCCOCCOCCOCCN ; CC(C)(C)OC(=O)NCCOCCOCCOCCNCC(=O)N1CCN(C(=O)c2cc(Cc3n[nH]c(=O)c4ccccc34)ccc2F)CC1 ; NCCOCCOCCOCCNCC(=O)N1CCN(C(=O)c2cc(Cc3n[nH]c(=O)c4ccccc34)ccc2F)CC1 ; Cc1sc(CN2CCCC2c2nnc3n2CCCCC3)nc1C12CC(C(=O)O)(C1)C2 ; Cc1sc(CN2CCCC2c2nnc3n2CCCCC3)nc1C12CC(C(=O)NCCOCCOCCOCCNCC(=O)N3CCN(C(=O)c4cc(Cc5n[nH]c(=O)c6ccccc56)ccc4F)CC3)(C1)C2]

### Step 1: Synthesis of Intermediate 1-3

To a solution of Intermediate 1-1 (0.2 g, 451.60 μmol, 1 eq) and KI (74.97 mg, 451.60 μmol, 1 eq) in DMF (1 mL) and ACN (1 mL) was added Intermediate 1-2 (145.24 mg, 496.76 μmol, 1.1 eq) and DIEA (233.46 mg, 1.81 mmol, 314.63 μL, 4 eq). The mixture was stirred at 60° C. for 2 hrs. The mixture was concentrated under vacuum. The crude product was purified by reversed-phase HPLC (0.1% NH₃H2O condition) and concentrated under vacuum to remove MeCN and lyophilized. The product was repurified by prep-HPLC (column: Phenomenex luna C18 150*25 mm*10 um; mobile phase: [water (FA)-ACN]; gradient: 12%-42% B over 9 min) and dried by lyophilization to afford Intermediate 1-3 (100 mg, 143.11 mol, 31.69% yield) as a colorless oil. LCMS (Method G): Rt=0.512 min, [M+H]⁺=699.4.

### Step 2: Synthesis of Intermediate 1-4

A mixture of Intermediate 1-3 (100 mg, 143.11 μmol, 1 eq) in DCM (1 mL) was added HCl/dioxane (2 M, 1 mL, 13.98 eq). The mixture was stirred at 25° C. for 0.5 hr. The mixture was concentrated under vacuum to afford Intermediate 1-4 (90 mg, crude, HCl salt) as a white solid. LCMS (Method E): Rt=0.355 min, [M+H]⁺=599.4.

### Step 3: Synthesis of I-194

A mixture of Intermediate 1-5 (35 mg, 69.58 μmol, 1 eq), Intermediate 1-4 (44.19 mg, 69.58 mol, 1 eq, HCl salt) and HOAt (9.47 mg, 69.58 μmol, 9.73 μL, 1 eq) in DMF (1 mL) was added EDCI (26.68 mg, 139.16 μmol, 2 eq) and NMM (35.19 mg, 347.90 μmol, 38.25 μL, 5 eq). The mixture was stirred at 25° C. for 1.5 hrs. The mixture was poured into MeOH (1 mL). The residue was purified twice by prep-HPLC (column: Phenomenex luna C18 150*25 mm*10 um; mobile phase: [water (FA)-ACN]; gradient: 8%-38% B over 9 min) and dried by lyophilization to afford I-194 (15.07 mg, 14.81 μmol, 21.29% yield, 99.0% purity) as an off-white oil. LCMS (Method E): Rt=0.400 min, [M+H]⁺=1008.6. SFC: Rt=3.175 min, 4.454 min. ¹H NMR (400 MHz, DMSO-d₆) δ=12.59 (s, 1H), 8.26 (d, J=6.8 Hz, 1H), 8.20-8.16 (m, 1H), 7.98-7.93 (m, 1H), 7.92-7.86 (m, 1H), 7.86-7.78 (m, 2H), 7.49-7.40 (m, 1H), 7.36 (br s, 1H), 7.28-7.18 (m, 1H), 4.33 (s, 2H), 4.28-4.13 (m, 2H), 3.95-3.94 (m, 1H), 3.74 (br d, J=14.8 Hz, 2H), 3.58 (br s, 2H), 3.54 (br s, 2H), 3.50 (br s, 8H), 3.46 (br d, J=4.4 Hz, 4H), 3.22-3.15 (m, 6H), 2.88-2.80 (m, 2H), 2.67 (br d, J=2.0 Hz, 3H), 2.38-2.28 (m, 5H), 2.27-2.15 (m, 7H), 1.94-1.48 (m, 10H). ¹⁹F NMR (377 MHz, DMSO-d6) δ=−119.7.

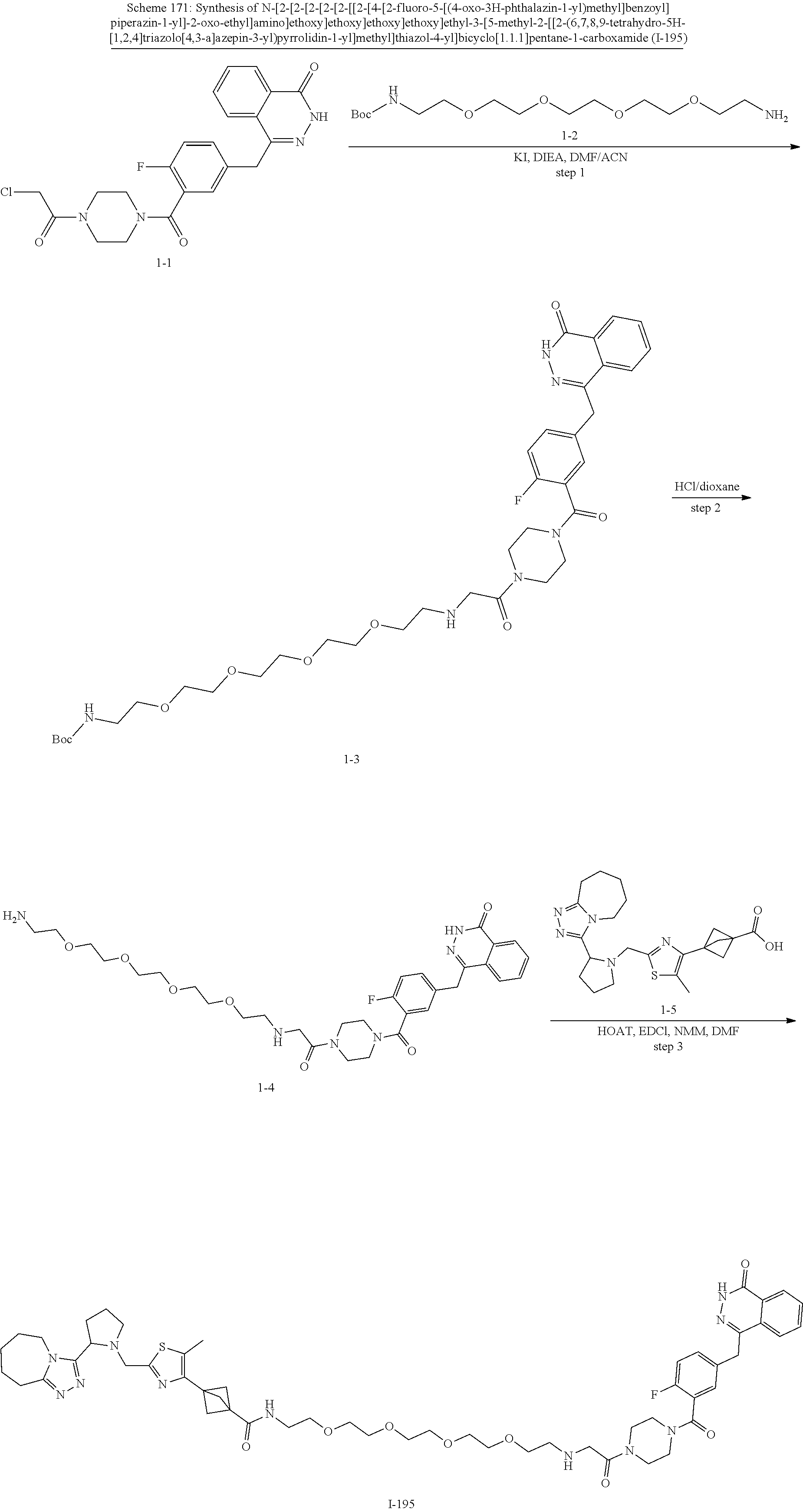 [SMILES: O=C(CCl)N1CCN(C(=O)c2cc(Cc3n[nH]c(=O)c4ccccc34)ccc2F)CC1 ; CC(C)(C)OC(=O)NCCOCCOCCOCCOCCN ; CC(C)(C)OC(=O)NCCOCCOCCOCCOCCNCC(=O)N1CCN(C(=O)c2cc(Cc3n[nH]c(=O)c4ccccc34)ccc2F)CC1 ; NCCOCCOCCOCCOCCNCC(=O)N1CCN(C(=O)c2cc(Cc3n[nH]c(=O)c4ccccc34)ccc2F)CC1 ; Cc1sc(CN2CCCC2c2nnc3n2CCCCC3)nc1C12CC(C(=O)O)(C1)C2 ; Cc1sc(CN2CCCC2c2nnc3n2CCCCC3)nc1C12CC(C(=O)NCCOCCOCCOCCOCCNCC(=O)N3CCN(C(=O)c4cc(Cc5n[nH]c(=O)c6ccccc56)ccc4F)CC3)(C1)C2]

### Step 1: Synthesis of Intermediate 1-3

To a solution of Intermediate 1-1 (0.2 g, 451.60 μmol, 1 eq) and KI (74.97 mg, 451.60 μmol, 1 eq) in DMF (1 mL) and ACN (1 mL) was added Intermediate 1-2 (167.12 mg, 496.76 μmol, 1.1 eq) and DIEA (233.46 mg, 1.81 mmol, 314.63 μL, 4 eq). The mixture was stirred at 60° C. for 2 hrs. The mixture was concentrated under vacuum. The crude product was purified by reversed-phase HPLC (0.1% NH₃·H₂O condition) and concentrated under vacuum to remove MeCN and dried by lyophilization. Then the product was repurified by prep-HPLC (column: Phenomenex luna C18 150*25 mm*10 um; mobile phase: [water (FA)-ACN]; gradient: 12%-42% B over 9 min) and dried by lyophilization to afford Intermediate 1-3 (150 mg, 201.93 μmol, 44.71% yield) as a colorless oil. LCMS (Method G): Rt=0.512 min, [M+H]⁺=743.6.

### Step 2: Synthesis of Intermediate 1-4

A mixture of Intermediate 1-3 (150 mg, 201.93 μmol, 1 eq) in DCM (1 mL) was added HCl/dioxane (2 M, 1 mL, 9.90 eq). The mixture was stirred at 25° C. for 0.5 hr. The mixture was concentrated under vacuum to afford Intermediate 1-4 (140 mg, crude, HCl salt) as a white solid. LCMS (Method E): Rt=0.370 min, [M+H]⁺=643.4.

### Step 3: Synthesis of I-195

To a mixture of Intermediate 1-5 (35 mg, 69.58 μmol, 1 eq), Intermediate 1-4 (47.26 mg, 69.58 μmol, 1 eq, HCl salt) and HOAt (9.47 mg, 69.58 μmol, 9.73 μL, 1 eq) in DMF (1 mL) was added EDCI (26.68 mg, 139.16 μmol, 2 eq) and NMM (35.19 mg, 347.90 μmol, 38.25 μL, 5 eq). The mixture was stirred at 25° C. for 1.5 hrs. The mixture was poured into MeOH (1 mL). The residue was purified twice by prep-HPLC (column: Phenomenex luna C18 150*25 mm*10 um; mobile phase: [water (FA)-ACN]; gradient: 10%-40% B over 9 min) and dried by lyophilized to afford I-195 (18.49 mg, 16.81 μmol, 24.16% yield, 95.6% purity, formic acid salt) as yellow oil. LCMS (Method E): Rt=0.423 min, [M+H]⁺=1052.6. SFC: Rt=0.769 min, 1.243 min. ¹H NMR (400 MHz, METHANOL-d₄) δ=8.49 (s, 1H), 8.37 (d, J=7.4 Hz, 1H), 7.98-7.92 (m, 1H), 7.91-7.80 (m, 2H), 7.53-7.47 (m, 1H), 7.38-7.32 (m, 1H), 7.18-7.14 (m, 1H), 4.59 (br d, J=3.6 Hz, 3H), 4.44-4.28 (m, 4H), 4.04-3.85 (m, 3H), 3.83-3.70 (m, 5H), 3.69-3.60 (m, 11H), 3.55 (br s, 4H), 3.38 (br d, J=5.6 Hz, 2H), 3.17-3.12 (m, 1H), 3.00-2.90 (m, 2H), 2.49-2.42 (m, 1H), 2.40 (s, 3H), 2.37 (s, 5H), 2.35-2.27 (m, 1H), 2.04 (s, 8H), 2.01-1.95 (m, 2H), 1.95-1.88 (m, 2H), 1.80-1.67 (m, 3H). ¹⁹F NMR (377 MHz, METHANOL-d₄) δ=−120.6.

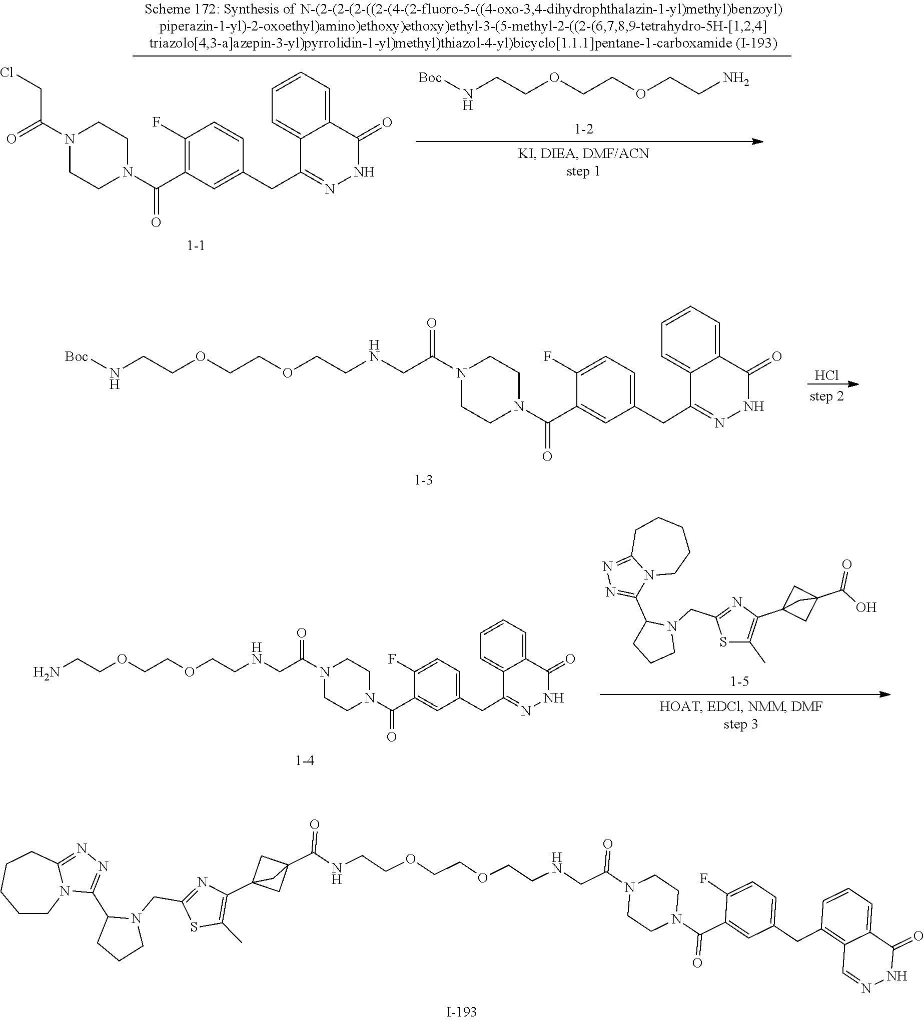

### Step 1: Synthesis of Intermediate 1-3

To a solution of Intermediate 1-1 (0.2 g, 451.60 μmol, 1 eq) and KI (74.97 mg, 451.60 μmol, 1 eq) in DMF (1 mL) and ACN (1 mL) was added Intermediate 1-2 (123.35 mg, 496.76 μmol, 1.1 eq) and DIEA (233.46 mg, 1.81 mmol, 314.63 μL, 4 eq). The mixture was stirred at 60° C. for 2 hr. The mixture was concentrated under vacuum. The crude product was purified by reversed-phase HPLC (0.1% NH₃·H₂O condition) and concentrated under vacuum to remove MeCN and dried by lyophilization. The residue was purified by prep-HPLC (column: Phenomenex luna C18 150*25 mm*10 um; mobile phase: [water(FA)-ACN]; gradient: 12%-42% B over 9 min) and dried by lyophilization to give Intermediate 1-3 (110 mg, 168.01 μmol, 37.20% yield) as colorless oil. LCMS (Method E): Rt=0.431 min, (M+H)*=655.3.

### Step 2: Synthesis of Intermediate 1-4

To a mixture of Intermediate 1-3 (110 mg, 168.01 μmol, 1 eq) in DCM (1 mL) was added HCl/dioxane (2 M, 1 mL, 11.90 eq) it was stirred at 25° C. for 0.5 hr. The mixture was concentrated under vacuum to give Intermediate 1-4 (0.1 g, crude, HCl salt) as a white solid. LCMS (Method E): Rt=0.354 min, (M+H)⁺=555.3.

### Step 3: Synthesis of I-193

To a mixture of Intermediate 1-5 (35 mg, 69.58 μmol, 1 eq), Intermediate 1-4 (41.13 mg, 69.58 μmol, 1 eq, HCl salt) and HOAt (9.47 mg, 69.58 μmol, 9.73 μL, 1 eq) in DMF (1 mL) was added EDCI (26.68 mg, 139.16 μmol, 2 eq) and NMM (35.19 mg, 347.90 μmol, 38.25 μL, 5 eq). The mixture was stirred at 25° C. for 1.5 hrs. The mixture was poured into MeOH (1 mL). The residue was purified by prep-HPLC (column: Phenomenex luna C18 150*25 mm*10 um; mobile phase: [water(FA)-ACN]; gradient: 8%-38% B over 9 min) and dried by lyophilization to give I-193 (22.99 mg, 23.54 μmol, 33.84% yield, 98.7% purity, formic acid salt) as an off-white solid. LCMS (Method E): Rt=0.405 min, (M+H)⁺=964.7. SFC: Rt=1.582 min, 1.973 min. ¹H NMR (400 MHz, DMSO-d6) δ=12.59 (s, 1H), 8.26 (d, J=7.2 Hz, 1H), 8.18 (br d, J=4.0 Hz, 2H), 7.99-7.93 (m, 1H), 7.89-7.88 (m, 1H), 7.86-7.77 (m, 2H), 7.48-7.41 (m, 1H), 7.37 (br d, J=3.6 Hz, 1H), 7.25-7.21 (m, 1H), 4.33 (s, 2H), 4.28-4.11 (m, 3H), 3.98-3.92 (m, 2H), 3.74 (br d, J=15.2 Hz, 4H), 3.57 (br s, 3H), 3.54 (br s, 2H), 3.50 (br s, 7H), 3.19 (br d, J=4.8 Hz, 5H), 2.84-2.83 (m, 2H), 2.73-2.64 (m, 2H), 2.33 (s, 4H), 2.23 (s, 6H), 1.94-1.83 (m, 3H), 1.83-1.48 (m, 7H). ¹⁹F NMR (377 MHz, DMSO-d6) δ=−119.6.

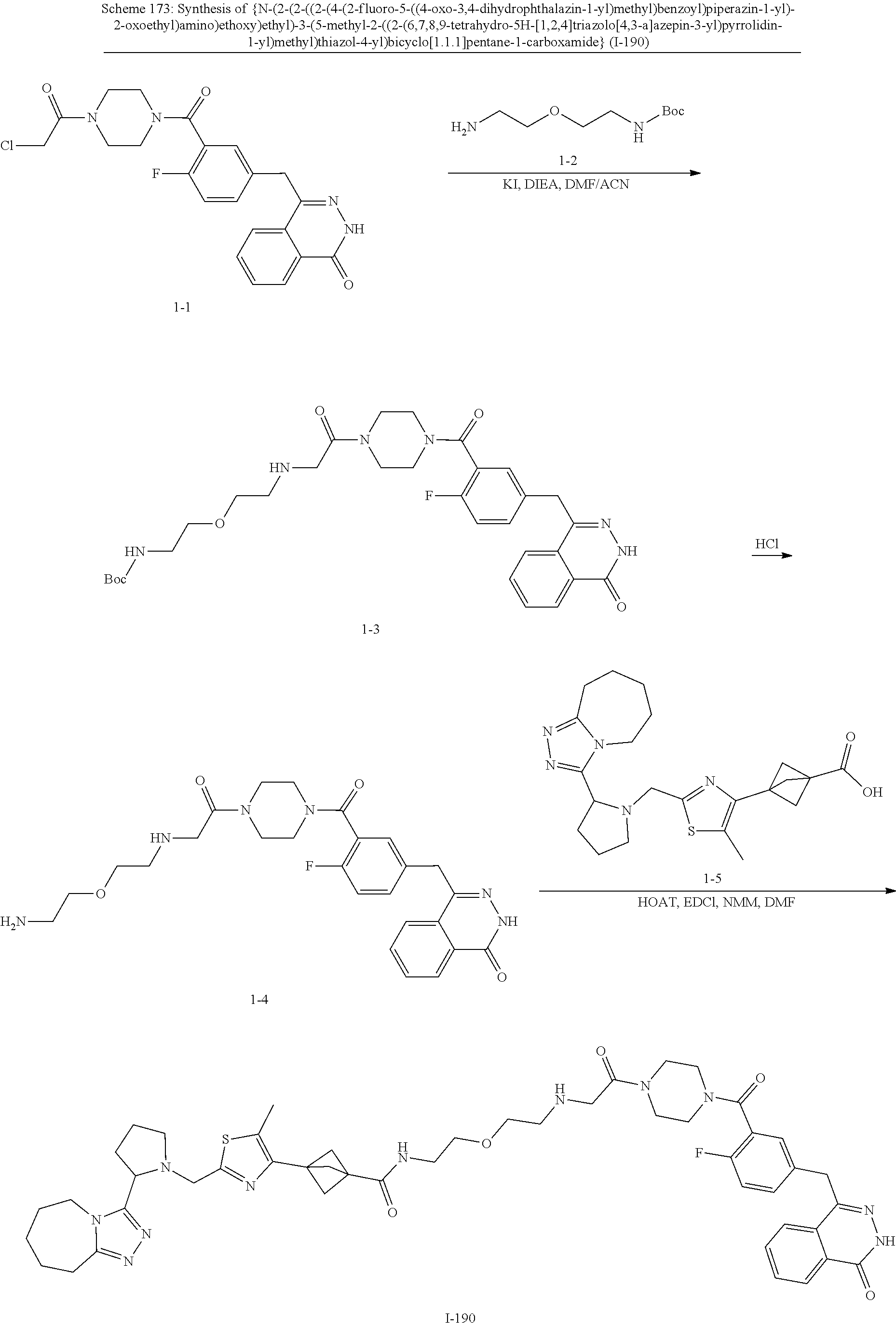

### Step 1: Synthesis of Intermediate 1-3

To a solution of Intermediate 1-1 (0.2 g, 451.60 μmol, 1 eq) in DMF (1 mL) and ACN (1 mL) was added KI (74.97 mg, 451.60 μmol, 1 eq) and DIEA (233.46 mg, 1.81 mmol, 4 eq) and Intermediate 1-2 (101.47 mg, 496.76 μmol, 1.1 eq). The mixture was stirred at 60° C. for 1.5 hr. To the reaction mixture was added H₂O (2 mL) and it was extracted with EA (5 mL*5) and washed with brine (3 mL*4). The organic layer was dried with anhydrous Na₂SO₄, filtered and concentrated under vacuum to give the residue. The residue was purified by flash silica gel chromatography (ISCO; SepaFlash Silica Flash Column, Eluent of 0˜30% DCM/MeOH gradient). Intermediate 1-3 (0.09 g, 129.07 μmol, 28.5% yield, 87.58% purity) was obtained as a yellow oil. LCMS (Method E): Retention time: 0.426 min, (M+H)⁺=611.4.

### Step 2: Synthesis of Intermediate 1-4

To a solution of Intermediate 1-3 (45 mg, 73.69 μmol, 1 eq) in dioxane (0.5 mL) was added HCl/dioxane (2 M, 0.5 mL, 13.57 eq). The mixture was stirred at 25° C. for 1 h. The mixture was concentrated under vacuum to give Intermediate 1-4 (40 mg, crude, HCl salt) as a yellow oil. LCMS (Method E): Retention time: 0.363 min, (M+H)⁺=511.3.

### Step 3: Synthesis of I-190

To a solution of Intermediate 1-5 (30 mg, 70.17 μmol, 1 eq), HOAt (9.55 mg, 70.17 μmol, 9.82 L, 1 eq), EDCI (26.90 mg, 140.33 μmol, 2 eq) and NMM (35.49 mg, 350.83 μmol, 38.57 μL, 5 eq) in DMF (0.5 mL) was added Intermediate 1-4 (38.38 mg, 70.17 μmol, 1 eq, HCl salt). The mixture was stirred at 25° C. for 12 h. The mixture was filtered. The filtrate was purified by prep-HPLC (column: Phenomenex luna C18 150*25 mm*10 um; mobile phase: [water (FA)-ACN]; gradient: 10%-40% B over 9 min). The eluent was concentrated under vacuum and combined with another lot of product and lyophilized. I-190 (31.06 mg, 31.20 μmol, 44.4% yield, 97.0% purity, formic acid salt) was obtained as white solid. LCMS (Method E): Rt=0.404 min, (M+H)⁺=920.7. SFC: Rt=2.874 min, Rt=3.973 min. ¹H NMR (400 MHz, DMSO-d6) δ=12.61 (s, 1H), 8.27 (d, J=7.6 Hz, 1H), 8.15 (s, 1H), 8.00-7.80 (m, 4H), 7.51-7.33 (m, 2H), 7.24 (m, 1H), 4.37-4.16 (m, 4H), 3.95 (m, 1H), 3.82-3.59 (m, 10H), 3.26-3.10 (m, 8H), 2.97-2.77 (m, 5H), 2.40-2.16 (m, 11H), 1.92-1.75 (m, 5H), 1.70-1.50 (m, 4H). ¹⁹F NMR (377 MHz, DMSO-d6) δ=−119.6.

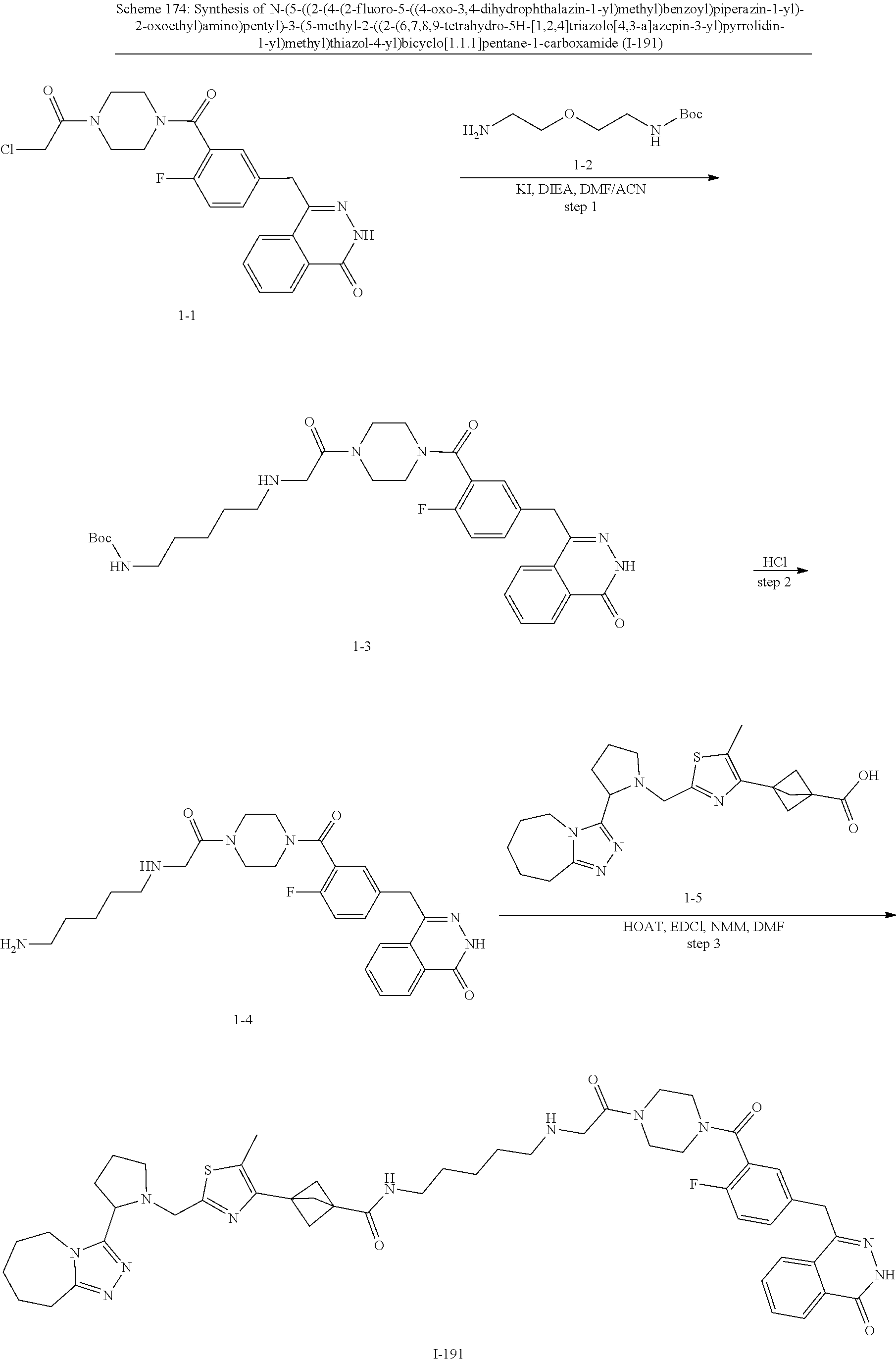

### Step 1: Synthesis of Intermediate 1-3

To a solution of Intermediate 1-1 (0.2 g, 451.60 μmol, 1 eq) and KI (74.97 mg, 451.60 μmol, 1 eq) in DMF (1 mL) and ACN (1 mL) was added Intermediate 1-2 (100.49 mg, 496.76 μmol, 103.39 μL, 1.1 eq) and DIEA (233.46 mg, 1.81 mmol, 314.63 μL, 4 eq). The mixture was stirred at 60° C. for 2 hr. The mixture was concentrated under vacuum. The crude product was purified by reversed-phase HPLC (0.1% NH₃·H₂O condition) and concentrated under vacuum to remove MeCN and dried by lyophilization. The residue was purified by prep-HPLC (column: Phenomenex luna C18 150*25 mm*10 um; mobile phase: [water(FA)-ACN]; gradient: 15%-45% B over 9 min) and dried by lyophilization to give Intermediate 1-3 (90 mg, 147.86 μmol, 32.74% yield) as colorless oil. LCMS (Method E): Rt=0.431 min, (M+H)⁺=609.4.

### Step 2: Synthesis of Intermediate 1-4

To a mixture of Intermediate 1-3 (90 mg, 147.86 μmol, 1 eq) in DCM (1 mL) was added HCl/dioxane (2 M, 1 mL, 13.53 eq) and the mixture was stirred at 25° C. for 0.5 hr. The mixture was concentrated under vacuum to give Intermediate 1-4 (80 mg, crude, HCl salt) as a white solid. LCMS: Rt=0.361 min, (M+H)⁺=509.3.

### Step 3: Synthesis of I-191

To a mixture of Intermediate 1-5 (20 mg, 46.78 μmol, 1 eq), Intermediate 1-4 (25.50 mg, 46.78 μmol, 1 eq, HCl salt) and HOAt (6.37 mg, 46.78 μmol, 6.54 μL, 1 eq) in DMF (1 mL) was added EDCI (17.93 mg, 93.55 μmol, 2 eq) and NMM (23.66 mg, 233.88 μmol, 25.71 μL, 5 eq). The mixture was stirred at 25° C. for 1.5 hrs. The mixture was poured into MeOH (1 mL). The residue was purified by prep-HPLC (column: Phenomenex luna C18 150*25 mm*10 um; mobile phase: [water(FA)-ACN]; gradient: 8%-38% B over 9 min) and dried by lyophilization to give I-191 (12.12 mg, 13.20 μmol, 28.22% yield, formic acid salt) as off-white solid. LCMS (Method E): Rt=0.418 min, (M+H)⁺=918.5. SFC: Rt=1.578 min, 1.955 min. ¹H NMR (400 MHz, DMSO-d6) δ=12.59 (s, 1H), 8.26 (d, J=7.6 Hz, 1H), 8.22 (s, 1H), 7.99-7.93 (m, 1H), 7.92-7.79 (m, 2H), 7.74-7.73 (m, 1H), 7.48-7.40 (m, 1H), 7.36 (br s, 1H), 7.25-7.21 (m, 1H), 4.33 (s, 2H), 4.29-4.12 (m, 2H), 3.95-3.94 (m, 1H), 3.74 (br d, J=15.2 Hz, 1H), 3.66-3.49 (m, 8H), 3.23-3.08 (m, 6H), 3.02 (br d, J=6.0 Hz, 2H), 2.90-2.81 (m, 2H), 2.37-2.31 (m, 4H), 2.22 (s, 6H), 2.18-2.12 (m, 1H), 1.94-1.83 (m, 3H), 1.80-1.52 (m, 6H), 1.47-1.33 (m, 4H), 1.30-1.22 (m, 2H), 1.07-1.03 (m, 1H). ¹⁹F NMR (377 MHz, DMSO-d6) δ=−119.7.

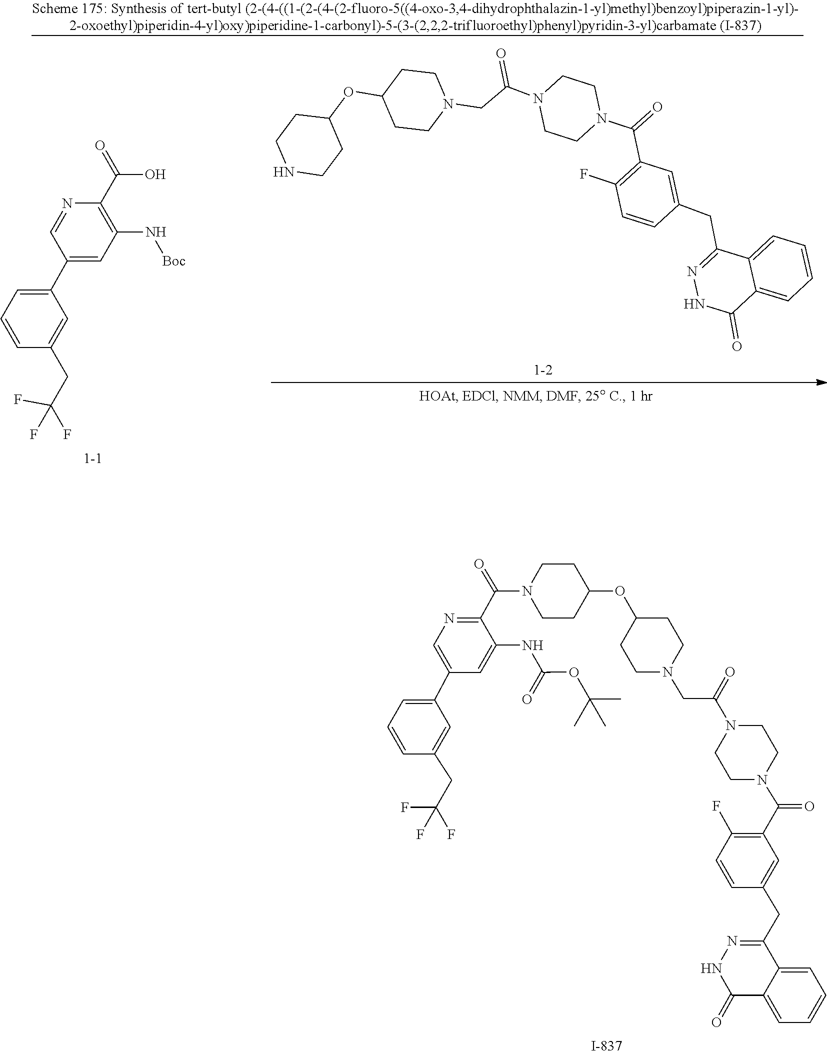 [SMILES: CC(C)(C)OC(=O)Nc1cc(-c2cccc(CC(F)(F)F)c2)cnc1C(=O)O ; O=C(CN1CCC(OC2CCNCC2)CC1)N1CCN(C(=O)c2cc(Cc3n[nH]c(=O)c4ccccc34)ccc2F)CC1 ; CC(C)(C)OC(=O)Nc1cc(-c2cccc(CC(F)(F)F)c2)cnc1C(=O)N1CCC(OC2CCN(CC(=O)N3CCN(C(=O)c4cc(Cc5n[nH]c(=O)c6ccccc56)ccc4F)CC3)CC2)CC1]

To a solution of Intermediate 1-1 (100 mg, 252.30 μmol, 1 eq) in DMF (1 mL) was added HOAt (34.34 mg, 252.30 μmol, 35.29 μL, 1 eq), EDCI (145.10 mg, 756.89 μmol, 3 eq), NMM (127.59 mg, 1.26 mmol, 138.69 μL, 5 eq) and Intermediate 1-2 (149.03 mg, 252.30 μmol, 1 eq). The mixture was stirred at 25° C. for 1 hr. The mixture was washed with H₂O (1 mL) and extract with EA (2 mL*3). The combined organic layers were dried over anhydrous sodium sulfate, filtered and concentrated under reduced pressure to give a residue. The residue was purified by prep-HPLC (column: CD01-Phenomenex luna C18 150*25*10 um; mobile phase: [water (FA)-ACN]; gradient: 25%-555% B over 10 min), the eluent was concentrated under reduced pressure to remove ACN and lyophilized to give I-837 (19.03 mg, 19.50 μmol, 62.99% yield, 99.303% purity) as a white solid. LCMS (Method D): Rt=0.411 min, [M+H]⁺=969.4. ¹H NMR (400 MHz, METHANOL-d₄) δ=8.55 (d, J=1.2 Hz, 1H), 8.45 (s, 1H), 8.42 (d, J=1.6 Hz, 1H), 8.37 (d, J=8.4 Hz, 1H), 7.98-7.92 (m, 1H), 7.90-7.81 (m, 2H), 7.72-7.67 (m, 2H), 7.57-7.48 (m, 2H), 7.45 (d, J=8.0 Hz, 1H), 7.37 (m, 1H), 7.17 (m, 1H), 4.39 (s, 2H), 4.09-4.02 (m, 1H), 3.81 (s, 2H), 3.75 (d, J=4.8 Hz, 1H), 3.68 (s, 2H), 3.65 (s, 3H), 3.61 (d, J=11.2 Hz, 3H), 3.54 (d, J=4.4 Hz, 3H), 3.49 (d, J=4.4 Hz, 1H), 3.38-3.33 (m, 2H), 3.06-2.95 (m, 2H), 2.75-2.56 (m, 2H), 2.00-1.89 (m, 4H), 1.79-1.65 (m, 4H), 1.53 (s, 9H). ¹⁹F NMR (376 MHz, METHANOL-d₄) δ=−67.41, −120.79.

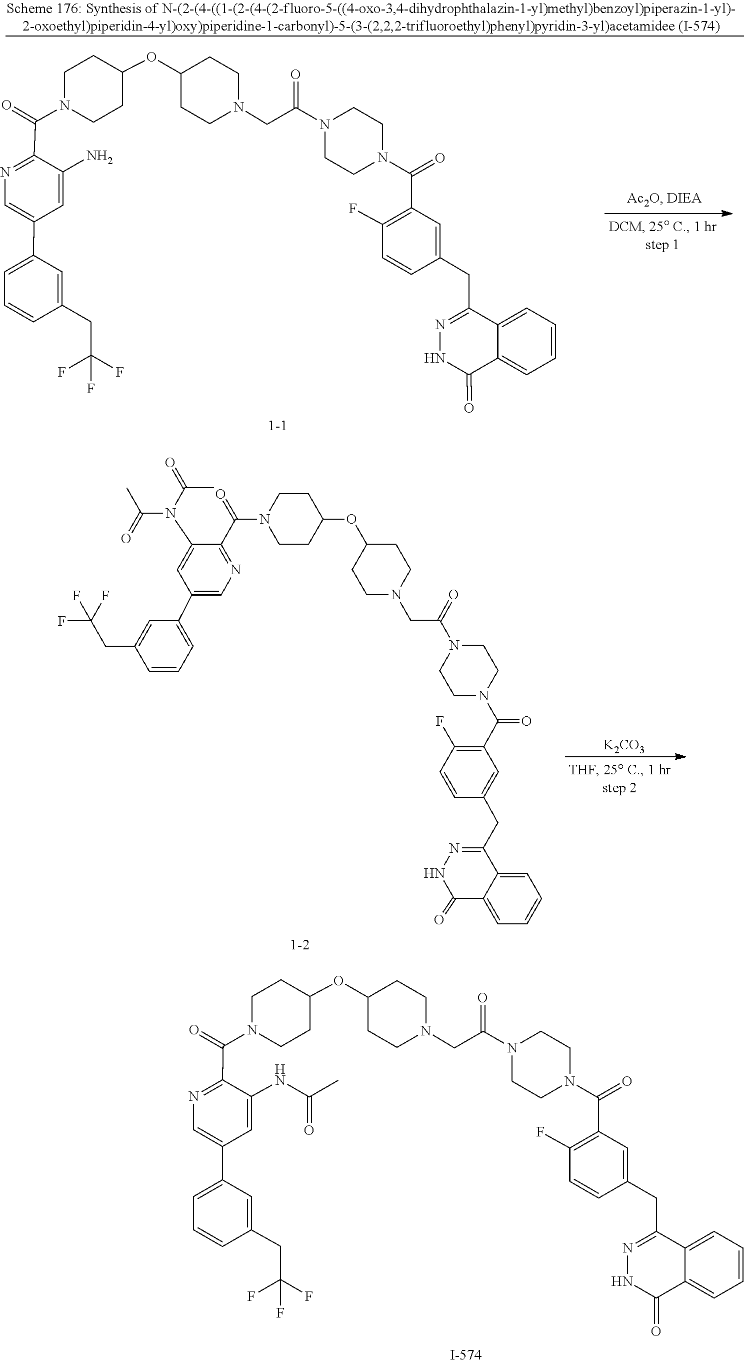

### Step 1: Synthesis of Intermediate 1-2

To a solution of Intermediate 1-1 (40 mg, 44.18 μmol, 1 eq, HCl salt) in DCM (0.4 mL) was added Ac₂O (5.64 mg, 55.25 μmol, 5.19 μL, 1.25 eq) and DIEA (17.84 mg, 138.04 μmol, 24.04 μL, 3.12 eq). The mixture was stirred at 25° C. for 1 hr. The mixture was washed with water (1 mL) and extracted with EA (2 mL*3). The combined organic layers were dried over sodium sulfate, filtered and concentrated under reduced pressure to give Intermediate 1-2 (60 mg, crude) as a yellow oil. LCMS (Method D): Rt=0.395 min, [M+H]⁺=953.2.

### Step 2: Synthesis of I-574

To a solution of Intermediate 1-2 (60 mg, 62.96 μmol, 1 eq) in THF (0.6 mL) was added K₂CO₃ (17.40 mg, 125.92 μmol, 2 eq). The mixture was stirred at 25° C. for 1 hr. The mixture was diluted with water (1 mL) and extracted with EA (2 mL*3). The combined organic layers were dried over sodium sulfate, filtered and concentrated under reduced pressure to give a residue. The residue was purified by reversed phase HPLC (0.1% FA condition), the eluent was concentrated under reduced pressure to remove ACN and lyophilized to give I-574 (13.85 mg, 15.13 μmol, 24.03% yield, 99.504% purity) as a white solid. LCMS (Method D): Rt=0.357 min, [M+H]⁺=911.2. ¹H NMR (400 MHz, METHANOL-d₄) δ=8.65 (s, 1H), 8.56-8.47 (m, 1H), 8.37 (d, J=7.6 Hz, 1H), 8.27 (s, 1H), 7.99-7.92 (m, 1H), 7.90-7.81 (m, 2H), 7.72-7.66 (m, 2H), 7.56-7.44 (m, 3H), 7.38 (m, 1H), 7.17 (m, 1H), 4.39 (s, 2H), 4.09-3.99 (m, 1H), 3.83-3.73 (m, 3H), 3.73-3.57 (m, 8H), 3.57-3.46 (m, 4H), 3.38-3.33 (m, 2H), 3.06-2.91 (m, 2H), 2.76-2.50 (m, 2H), 2.17 (s, 3H), 2.02-1.88 (m, 4H), 1.70 (m, 4H). ¹⁹F NMR (376 MHz, METHANOL-d₄) δ=−67.42, −120.79.

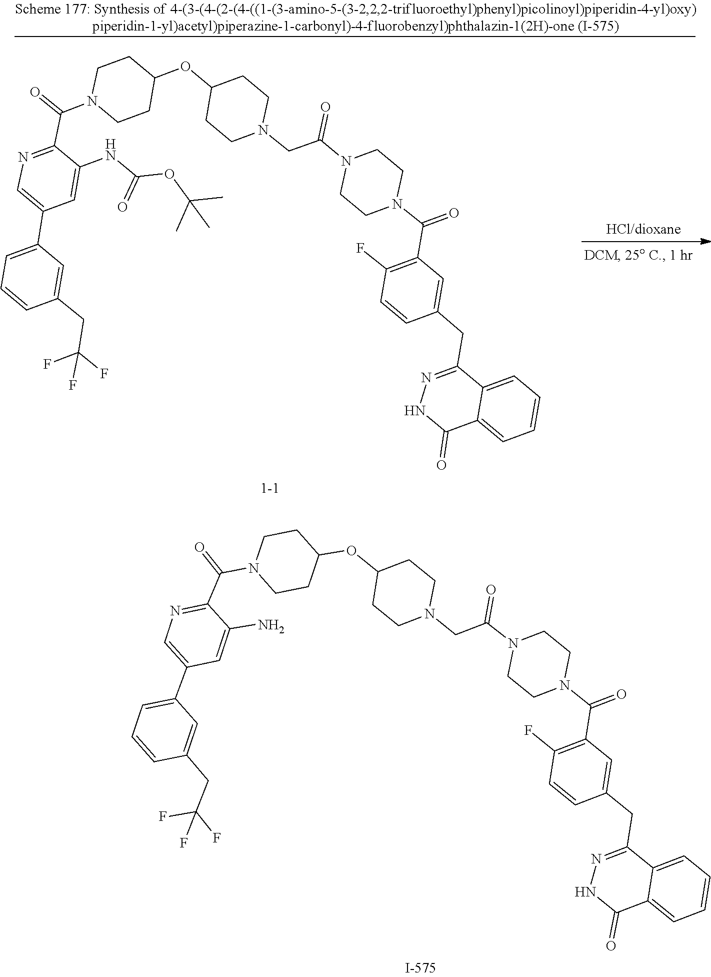 [SMILES: CC(C)(C)OC(=O)Nc1cc(-c2cccc(CC(F)(F)F)c2)cnc1C(=O)N1CCC(OC2CCN(CC(=O)N3CCN(C(=O)c4cc(Cc5n[nH]c(=O)c6ccccc56)ccc4F)CC3)CC2)CC1 ; Nc1cc(-c2cccc(CC(F)(F)F)c2)cnc1C(=O)N1CCC(OC2CCN(CC(=O)N3CCN(C(=O)c4cc(Cc5n[nH]c(=O)c6ccccc56)ccc4F)CC3)CC2)CC1]

To a solution of Intermediate 1-1 (35 mg, 36.12 μmol, 1 eq) in DCM (0.4 mL) was added HCl/dioxane (2 M, 0.4 mL). The mixture was stirred at 25° C. for 1 hr. The reaction mixture was concentrated under vacuum. The residue was purified by prep-HPLC (column: CD03-Welch Xtimate C18 150*25*5 um; mobile phase: [water(FA)-ACN]; gradient: 18%-48% B over 10 min), the eluent was concentrated under reduced pressure to remove ACN and lyophilized to give I-575 (18.11 mg, 19.75 μmol, 54.67% yield, 99.758% purity, FA salt) as a white solid. LCMS (Method D): Rt=0.375 min, [M+H]⁺=869.3. ¹H NMR (400 MHz, METHANOL-d₄) δ=8.44 (s, 1H), 8.40-8.34 (m, 1H), 8.09 (d, J=1.6 Hz, 1H), 7.98-7.92 (m, 1H), 7.90-7.81 (m, 2H), 7.65-7.60 (m, 2H), 7.50 (m, 2H), 7.45-7.35 (m, 3H), 7.17 (m, 1H), 4.39 (s, 2H), 4.12-4.02 (m, 1H), 3.84-3.75 (m, 3H), 3.74 (s, 1H), 3.70 (s, 4H), 3.63-3.56 (m, 4H), 3.56-3.50 (m, 2H), 3.48 (s, 1H), 3.38-3.33 (m, 2H), 3.10-2.97 (m, 2H), 2.83-2.56 (m, 2H), 1.97 (d, J=3.2 Hz, 3H), 1.91-1.84 (m, 1H), 1.82-1.66 (m, 3H), 1.65-1.56 (m, 1H). ¹⁹F NMR (376 MHz, METHANOL-d₄) δ=−67.43, −120.78.

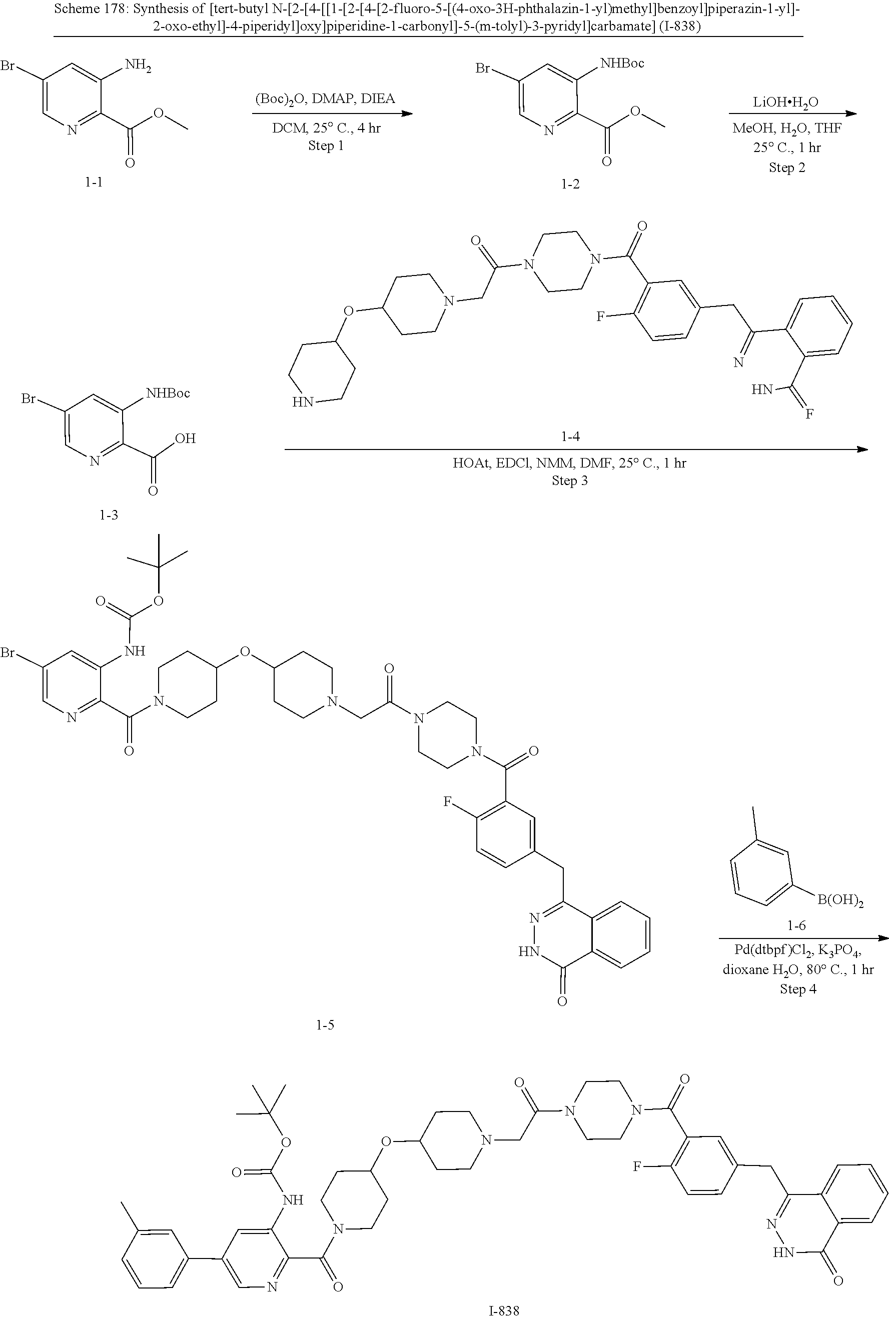

### Step 1: Synthesis of Intermediate 1-2.

To a mixture of intermediate 1-1 (2 g, 8.66 mmol, 1 eq) in DCM (20 mL) was added (Boc)₂O (1.89 g, 8.66 mmol, 1.99 mL, 1 eq), DMAP (105.75 mg, 865.63 μmol, 0.1 eq) and DIEA (2.24 g, 17.31 mmol, 3.02 mL, 2 eq) at 25° C., and the mixture was stirred at 25° C. for 4 hours. The reaction mixture was diluted with H₂O (30 mL) and extracted with DCM (20 mL×3), the combined organic layers were dried over Na₂SO₄, filtered and concentrated to give a residue. The residue was purified by column chromatography (SiO₂, PE/EA=1/0 to 7/3) and concentrated to give Intermediate 1-2 (1.42 g, 4.29 mmol, 49.54% yield, N/A purity) as a white solid. LCMS (Method D): Retention time: 0.413 min, [M+H]⁺=331.0. ¹H NMR (400 MHz, METHANOL-d₄) δ=9.07 (d, J=2.0 Hz, 1H), 8.33 (d, J=2.0 Hz, 1H), 3.97 (s, 3H), 1.55 (s, 9H).

### Step 2: Synthesis of Intermediate 1-3.

To a mixture of Intermediate 1-2 (1.42 g, 4.29 mmol, 1 eq) in H₂O (5 mL), THF (5 mL) and MeOH (5 mL) was added LiOH·H₂O (539.81 mg, 12.86 mmol, 3 eq) at 25° C., and the mixture was stirred at 25° C. for 1 h. The reaction mixture was adjusted to pH=5 by aq. HCl (1 N) and extracted with EtOAc (15 mL×3), the combined organic layers were dried over Na₂SO₄, filtered and concentrated to give Intermediate 1-3 (1.17 g, 3.69 mmol, 86.04% yield) as a white solid. LCMS (Method E): Rt: 0.376 min, [M-tBu+H]⁺=260.9.

### Step 3: Synthesis of Intermediate 1-5.

To a mixture of intermediate 1-3 (640 mg, 2.02 mmol, 1.27 eq) in DMF (10 mL) was added intermediate 1-4 (1 g, 1.59 mmol, 1 eq, HCl salt), EDCI (917.01 mg, 4.78 mmol, 3 eq), HOAt (217.03 mg, 1.59 mmol, 223.05 μL, 1 eq) and NMM (806.41 mg, 7.97 mmol, 876.53 μL, 5 eq) at 25° C., and the mixture was stirred at 25° C. for 1 h. The reaction mixture was diluted with H₂O (20 mL) and extracted with EtOAc (20 mL×3). The combined organic layers were dried over Na₂SO₄, filtered and concentrated to give a residue. The residue was purified by column chromatography (SiO₂, EA/MeOH=1/0 to 7/3) and concentrated to give Intermediate 1-5 (0.9 g, 1.01 mmol, 63.43% yield) as a brown solid. LCMS (Method D): Retention time: 0.367 min, [M+H]⁺=889.2.

### Step 4: Synthesis of I-838.

To a mixture of intermediate 1-5 (50 mg, 56.19 μmol, 1 eq) in dioxane (0.5 mL) and H₂O (0.1 mL) was added intermediate 1-6 (9.17 mg, 67.43 μmol, 1.2 eq), ditert-butyl(cyclopenta-1,4-dien-1-yl)phosphane;dichloropalladium;iron (3.66 mg, 5.62 μmol, 0.1 eq) and K₃PO₄ (35.78 mg, 168.58 μmol, 3 eq) at 25° C., the mixture was degassed and purged with N₂ 3 times, and then the mixture was stirred at 80° C. for 1 h under N₂ atmosphere. The reaction mixture was diluted with H₂O (2 mL) and extracted with DCM (2 mL×3), the combined organic layers were dried over Na₂SO₄, filtered and concentrated to give a residue. The residue was purified by reversed phase HPLC (0.1% FA condition) and lyophilized to give I-838 (18.66 mg, 20.71 μmol, 36.86% yield, 100% purity) as a white solid. LCMS (Method D): Retention time: 0.405 min, [M+H]⁺=901.3. ¹H NMR (400 MHz, METHANOL-d4) δ=8.54-8.50 (m, 1H), 8.44-8.32 (m, 2H), 7.99-7.92 (m, 1H), 7.91-7.80 (m, 2H), 7.53-7.46 (m, 3H), 7.42-7.35 (m, 2H), 7.28 (d, J=7.6 Hz, 1H), 7.17 (t, J=8.8 Hz, 1H), 4.39 (s, 2H), 4.12-4.00 (m, 1H), 3.80 (s, 2H), 3.77-3.72 (m, 1H), 3.68 (d, J=4.4 Hz, 3H), 3.63-3.46 (m, 5H), 3.45-3.33 (m, 4H), 2.96-2.76 (m, 2H), 2.44 (s, 5H), 2.00-1.86 (m, 4H), 1.74-1.60 (m, 4H), 1.53 (s, 9H). ¹⁹F NMR (377 MHz, METHANOL-d₄), δ=−120.794.

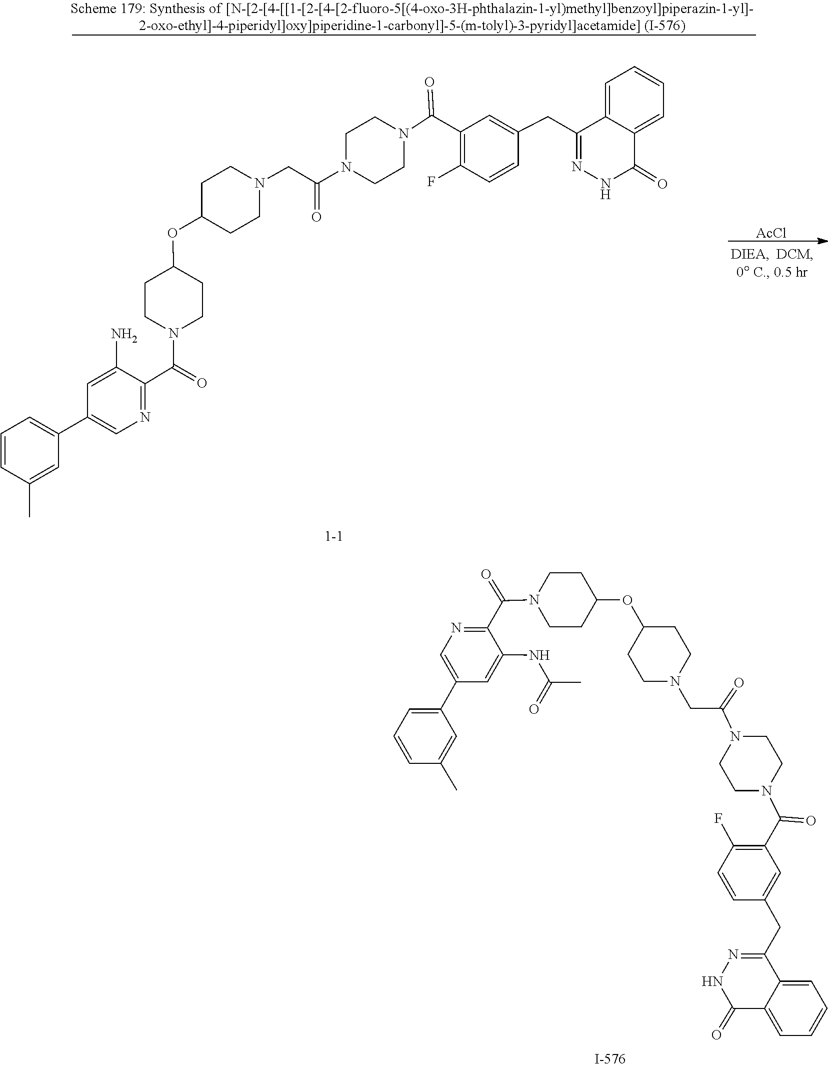 [SMILES: "AcCl" ; Cc1cccc(-c2cnc(C(=O)N3CCC(OC4CCN(CC(=O)N5CCN(C(=O)c6cc(Cc7n[nH]c(=O)c8ccccc78)ccc6F)CC5)CC4)CC3)c(N)c2)c1 ; CC(=O)Nc1cc(-c2cccc(C)c2)cnc1C(=O)N1CCC(OC2CCN(CC(=O)N3CCN(C(=O)c4cc(Cc5n[nH]c(=O)c6ccccc56)ccc4F)CC3)CC2)CC1]

To a mixture of Intermediate 1-1 (100 mg, 119.42 μmol, 1 eq, HCl salt) in DCM (1 mL) was added DIEA (30.87 mg, 238.84 μmol, 41.60 μL, 2 eq) and acetyl chloride (9.37 mg, 119.42 μmol, 8.49 μL, 1 eq) at 0° C., and the mixture was stirred at 0° C. for 0.5 h. The reaction mixture was diluted with H₂O (1 mL) and extracted with DCM (1 mL×3), the combined organic layers were dried over Na₂SO₄, filtered and concentrated to give a residue. The residue was purified by prep-HPLC (column: CD01-Phenomenex luna C18 150*25*10 um; mobile phase: [water (FA)-ACN]; gradient: 12%-42% B over 10 min) and lyophilized to give I-576 (21.65 mg, 24.27 μmol, 20.33% yield, 99.67% purity, FA salt) as a white solid. LCMS (Method D): Retention time: 0.342 min, [M+H]⁺=843.3. ¹H NMR (400 MHz, METHANOL-d4) δ=8.62 (s, 1H), 8.49 (s, 1H), 8.37 (d, J=8.0 Hz, 1H), 8.25 (s, 1H), 7.98-7.92 (m, 1H), 7.91-7.81 (m, 2H), 7.53-7.44 (m, 3H), 7.43-7.34 (m, 2H), 7.28 (d, J=7.6 Hz, 1H), 7.17 (t, J=9.2 Hz, 1H), 4.39 (s, 2H), 4.07-3.98 (m, 1H), 3.80 (s, 2H), 3.75 (s, 1H), 3.67 (s, 3H), 3.64-3.60 (m, 1H), 3.52 (s, 5H), 3.42 (s, 1H), 3.37-3.33 (m, 2H), 3.02-2.85 (m, 2H), 2.61-2.46 (m, 2H), 2.43 (s, 3H), 2.17 (s, 3H), 2.01-1.87 (m, 4H), 1.77-1.61 (m, 4H). ¹⁹F NMR (376 MHz, METHANOL-d4) δ=−120.785.

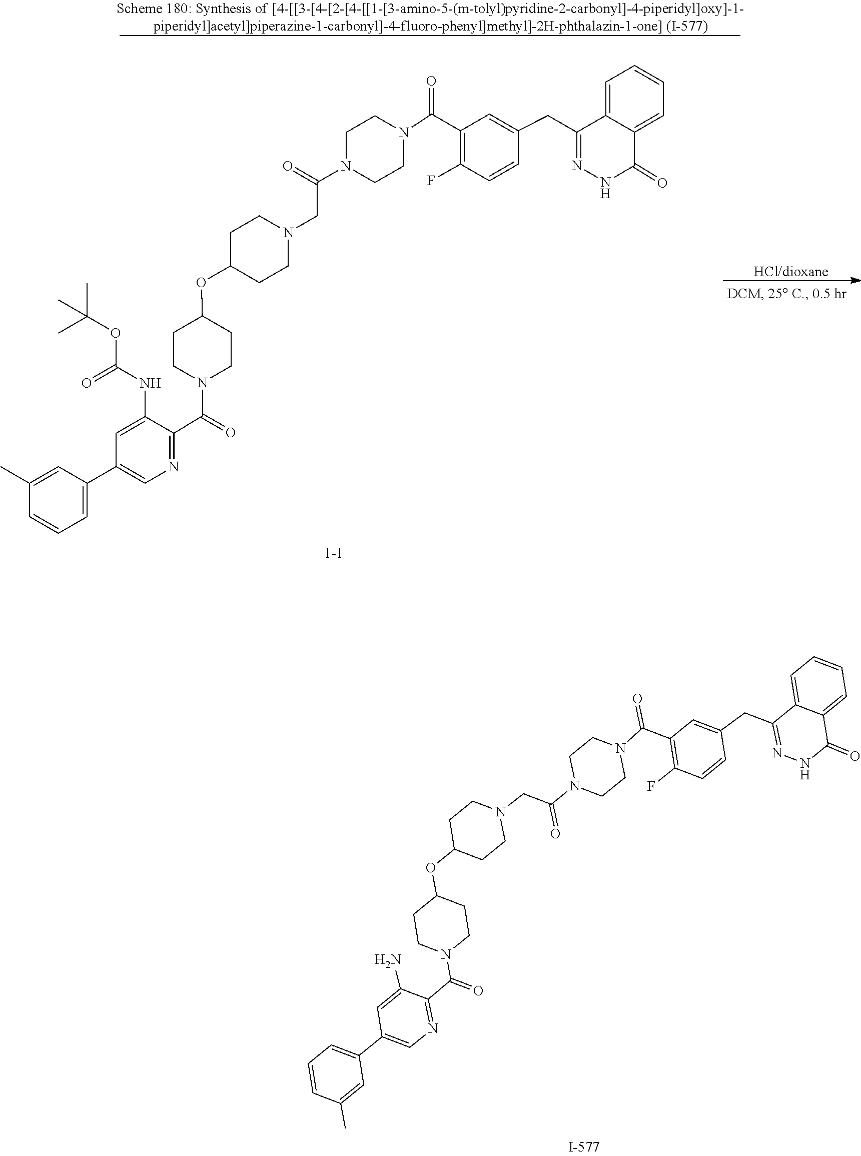 [SMILES: Cc1cccc(-c2cnc(C(=O)N3CCC(OC4CCN(CC(=O)N5CCN(C(=O)c6cc(Cc7n[nH]c(=O)c8ccccc78)ccc6F)CC5)CC4)CC3)c(NC(=O)OC(C)(C)C)c2)c1 ; "HCl/dioxane" ; Cc1cccc(-c2cnc(C(=O)N3CCC(OC4CCN(CC(=O)N5CCN(C(=O)c6cc(Cc7n[nH]c(=O)c8ccccc78)ccc6F)CC5)CC4)CC3)c(N)c2)c1]

To a mixture of intermediate 1-1 (30 mg, 33.30 μmol, 1 eq) in DCM (0.5 mL) was added HCl/dioxane (2 M, 0.5 mL, 30.03 eq) at 25° C., and the mixture was stirred at 25° C. for 0.5 h. I-577 (24.21 mg, 28.26 μmol, 84.86% yield, 97.730% purity, HCl salt) was obtained as a yellow solid. LCMS (Method D): Retention time: 0.337 min, [M+H]⁺=801.3. ¹H NMR (400 MHz, METHANOL-d₄) δ=8.40-8.35 (m, 1H), 8.24 (d, J=1.2 Hz, 1H), 7.98-7.92 (m, 1H), 7.91-7.78 (m, 3H), 7.55-7.46 (m, 3H), 7.45-7.30 (m, 3H), 7.18 (t, J=9.2 Hz, 1H), 4.39 (s, 2H), 4.33 (d, J=3.6 Hz, 1H), 4.26 (d, J=4.8 Hz, 1H), 3.97 (s, 1H), 3.92-3.76 (m, 5H), 3.73 (d, J=1.2 Hz, 1H), 3.70-3.63 (m, 1H), 3.57-3.43 (m, 5H), 3.37 (s, 3H), 3.20-3.08 (m, 1H), 2.44 (s, 3H), 2.34-2.17 (m, 1H), 2.07 (s, 3H), 2.01-1.92 (m, 2H), 1.91-1.80 (m, 1H), 1.77-1.65 (m, 2H). ¹⁹F NMR (376 MHz, METHANOL-d₄) δ=−120.770.

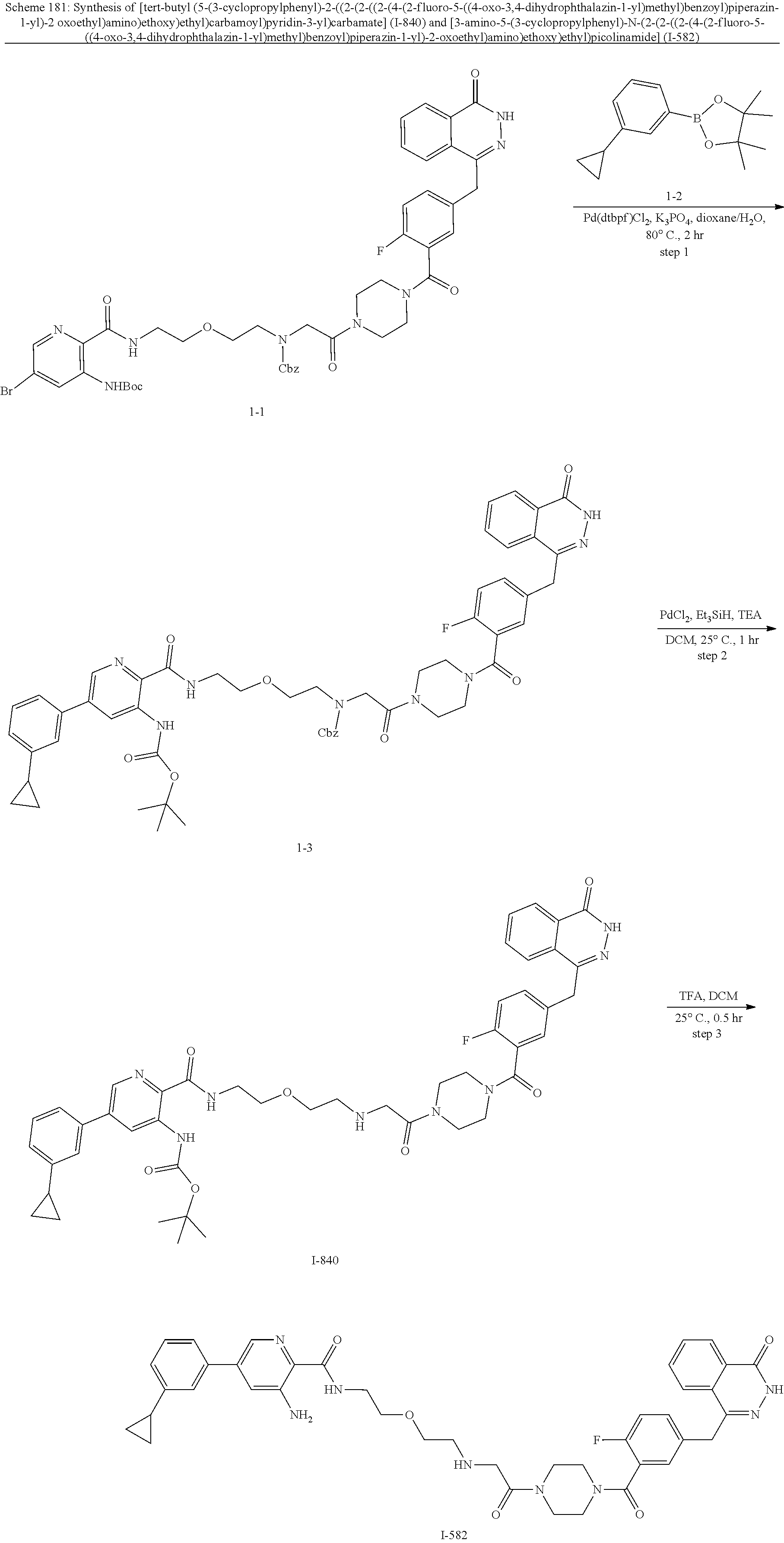 [SMILES: CC(C)(C)OC(=O)Nc1cc(Br)cnc1C(=O)NCCOCCN(CC(=O)N1CCN(C(=O)c2cc(Cc3n[nH]c(=O)c4ccccc34)ccc2F)CC1)C(=O)OCc1ccccc1 ; CC1(C)OB(c2cccc(C3CC3)c2)OC1(C)C ; CC(C)(C)OC(=O)Nc1cc(-c2cccc(C3CC3)c2)cnc1C(=O)NCCOCCN(CC(=O)N1CCN(C(=O)c2cc(Cc3n[nH]c(=O)c4ccccc34)ccc2F)CC1)C(=O)OCc1ccccc1 ; CC(C)(C)OC(=O)Nc1cc(-c2cccc(C3CC3)c2)cnc1C(=O)NCCOCCNCC(=O)N1CCN(C(=O)c2cc(Cc3n[nH]c(=O)c4ccccc34)ccc2F)CC1 ; Nc1cc(-c2cccc(C3CC3)c2)cnc1C(=O)NCCOCCNCC(=O)N1CCN(C(=O)c2cc(Cc3n[nH]c(=O)c4ccccc34)ccc2F)CC1]

### Step 1: Synthesis of Intermediate 1-3.

A mixture of Intermediate 1-1 (150 mg, 158.93 μmol, 1 eq), Intermediate 1-2 (42.68 mg, 174.82 μmol, 1.1 eq), K₃PO₄ (101.21 mg, 476.79 μmol, 3 eq), and Pd(dtbpf)Cl₂ (10.36 mg, 15.89 μmol, 0.1 eq) in dioxane (1 mL) and H₂O (0.1 mL) was degassed and purged with N₂ 3 times, and then the mixture was stirred at 80° C. for 1 hr under N₂ atmosphere. The reaction mixture was diluted with H₂O (2 mL) and extracted with EA (5 mL*3). The combined organic layers were dried over Na₂SO₄, filtered and concentrated under reduced pressure to give a residue. The residue was purified by flash silica gel chromatography (ISCO; 12 g SepaFlash Silica Flash Column, Eluent of 0˜5% Methanol/Ethyl gradient @60 mL/min), and the eluent was concentrated under reduced pressure to give a residue. Intermediate 1-3 (80 mg, 69.66 μmol, 43.83% yield, 85.433% purity) was obtained as a yellow solid. LCMS (Method D): Retention time: 0.576 min, [M-Boc+H]⁺=881.2. ¹H NMR (400 MHz, METHANOL-d₄) δ=9.03-8.87 (m, 1H), 8.47-8.29 (m, 2H), 7.94-7.79 (m, 3H), 7.49-7.28 (m, 8H), 7.25-7.21 (m, 2H), 7.13 (d, J=6.8 Hz, 2H), 5.15-5.10 (m, 1H), 4.99 (d, J=8.0 Hz, 1H), 4.38-4.30 (m, 2H), 4.28-4.20 (m, 2H), 3.74-3.71 (m, 1H), 3.68-3.60 (m, 5H), 3.56 (d, J=4.0 Hz, 7H), 3.40 (d, J=6.0 Hz, 1H), 3.24-3.08 (m, 2H), 2.03-1.95 (m, 1H), 1.54 (s, 9H), 1.04-0.96 (m, 2H), 0.78-0.72 (m, 2H).

### Step 2: Synthesis of I-840.

A mixture of Intermediate 1-3 (70 mg, 71.35 μmol, 1 eq), PdCl₂ (3.80 mg, 21.41 μmol, 0.3 eq) and TEA (14.44 mg, 142.70 μmol, 19.86 μL, 2 eq) in DCM (0.5 mL) was degassed and purged with N₂ 3 times, and then a solution of Et₃SiH (33.19 mg, 285.40 μmol, 45.58 μL, 4 eq) in DCM (0.2 mL) was added into the system at 10° C. The mixture was stirred at 25° C. for 1 hr under N₂ atmosphere. The reaction mixture was filtered and concentrated under reduced pressure to give a residue. The crude product was purified by reversed-phase HPLC (neutral condition), the eluent was concentrated and lyophilized to give I-840 (40 mg, 47.23 μmol, 66.19% yield, 100% purity) as a white solid. LCMS: Retention time (Method D): 0.451 min, [M+H]⁺=847.2. ¹H NMR (400 MHz, METHANOL-d₄) δ=8.97 (s, 1H), 8.45 (d, J=8.0 Hz, 1H), 8.38-8.34 (m, 1H), 7.95-7.90 (m, 1H), 7.87-7.78 (m, 2H), 7.49-7.32 (m, 5H), 7.17-7.06 (m, 2H), 4.84 (s, 1H), 4.36 (d, J=5.6 Hz, 2H), 3.74-3.56 (m, 11H), 3.53-3.44 (m, 3H), 3.21 (s, 1H), 2.85 (d, J=4.4 Hz, 2H), 2.05-1.92 (m, 1H), 1.54 (s, 9H), 1.04-0.94 (m, 2H), 0.79-0.70 (m, 2H). ¹⁹F NMR (376 MHz, METHANOL-d₄) δ=−120.61.

### Step 3: Synthesis of I-582

To a solution of I-840 (20 mg, 23.61 μmol, 1 eq) in DCM (0.2 mL) was added TFA (0.04 mL). The mixture was stirred at 25° C. for 0.5 hr. The reaction mixture was concentrated under reduced pressure to give a residue. The crude product was purified by reversed-phase HPLC (neutral condition), and the eluent was concentrated and lyophilized to give I-582 (17.33 mg, 19.83 μmol, 83.99% yield, 98.525% purity, TFA salt) as a white solid. LCMS (Method D): Retention time: 0.379 min, [M+H]⁺=747.3. ¹H NMR (400 MHz, METHANOL-d₄) δ=8.39-8.33 (m, 1H), 8.04 (d, J=15.6 Hz, 1H), 7.95-7.90 (m, 1H), 7.89-7.80 (m, 2H), 7.53-7.44 (m, 1H), 7.37-7.25 (m, 5H), 7.18-7.05 (m, 2H), 4.40-4.33 (m, 2H), 3.79-3.65 (m, 10H), 3.60 (s, 2H), 3.49 (s, 2H), 3.28-3.17 (m, 2H), 3.03-2.95 (m, 2H), 2.00-1.90 (m, 1H), 1.03-0.91 (m, 2H), 0.75-0.67 (m, 2H). ¹⁹F NMR (376 MHz, METHANOL-d₄) δ=−77.04, −120.62.

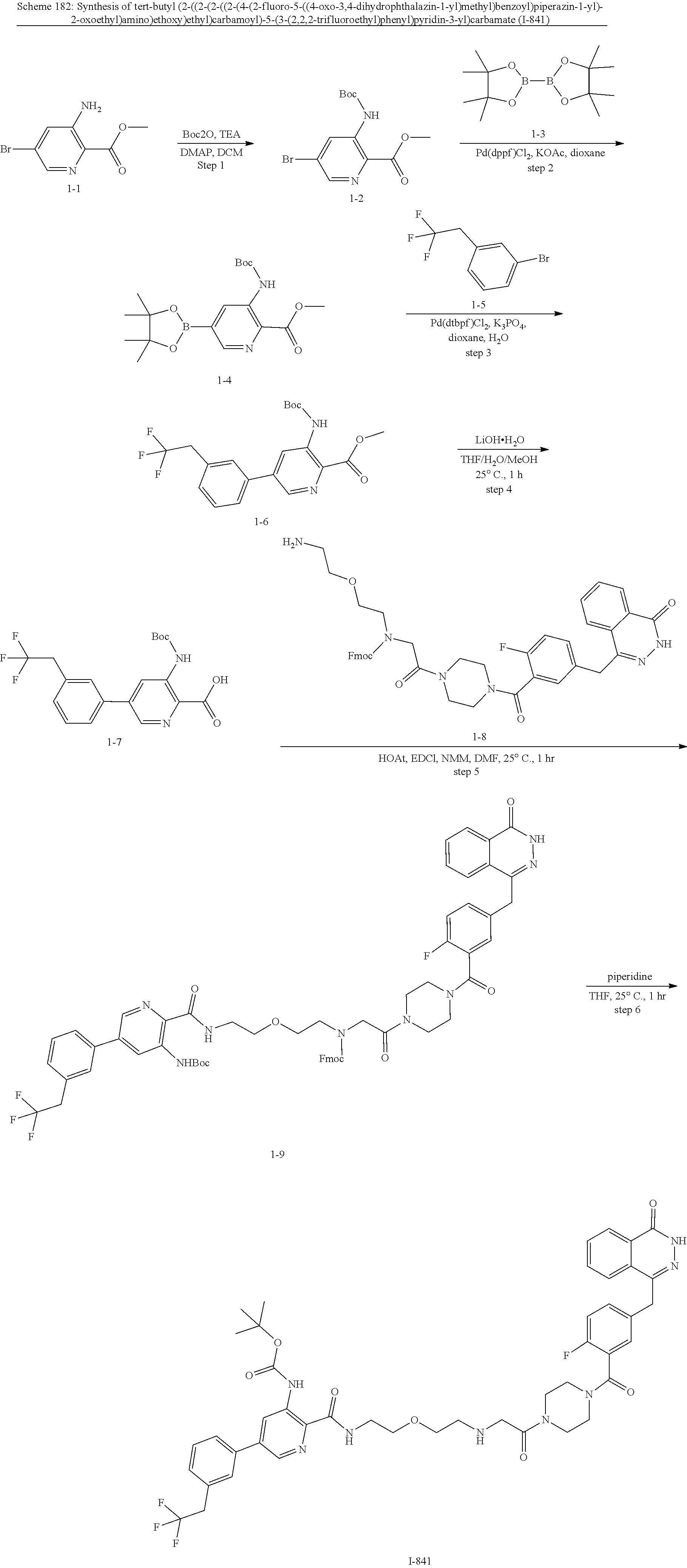

### Step 1: Synthesis of Intermediate 1-2

To a solution of Intermediate 1-1 (10 g, 43.28 mmol, 1 eq) in DCM (100 mL) was added Boc₂O (12.28 g, 56.27 mmol, 12.93 mL, 1.3 eq), DMAP (1.06 g, 8.66 mmol, 0.2 eq) and TEA (13.14 g, 129.84 mmol, 18.07 mL, 3 eq). The mixture was stirred at 25° C. for 1 hr. The reaction mixture was diluted with water (120 mL) and extracted with ethyl acetate (40 mL×3). The combined organic layers were washed with brine (40 mL×3) and dried over sodium sulfate, filtered and concentrated under reduced pressure to give a residue. The residue was purified by flash silica gel chromatography (ISCO; 330 g SepaFlash Silica Flash Column, Eluent of 0-50% EA:PE gradient @40 mL/min). The eluent was concentrated under reduced pressure to give a residue. Intermediate 1-2 (13 g, 33.46 mmol, 77.31% yield, 85.238% purity) was obtained as a white solid. LCMS (Method D): Rt=0.473 min, [M+H]⁺=330.9. ¹H NMR (400 MHz, CHLOROFORM-d) δ=10.21 (s, 1H), 9.14 (d, J=2.0 Hz, 1H), 8.37 (s, 1H), 4.02 (s, 3H), 1.55 (s, 9H).

### Step 2: Synthesis of Intermediate 1-4

To a mixture of Intermediate 1-2 (2 g, 6.04 mmol, 1 eq) in dioxane (20 mL) was added Intermediate 1-3 (4.60 g, 18.12 mmol, 3 eq), Pd(dppf)Cl₂ (441.90 mg, 603.93 μmol, 0.1 eq) and AcOK (1.78 g, 18.12 mmol, 3 eq) at 25° C., the mixture was degassed and purged with N₂ 3 times, and then the mixture was stirred at 100° C. for 1 hr. The reaction mixture was diluted with H₂O (30 mL) and extracted with DCM (20 mL×3), the combined organic layers were dried over Na₂SO₄, filtered and concentrated to give a residue. The crude product was purified by reversed-phase HPLC (0.1% FA condition) and lyophilized to give Intermediate 1-4 (1.9 g, crude) as a brown solid. LCMS (Method D): Rt=0.324 min, [M+H]⁺=297.1.

### Step 3: Synthesis of Intermediate 1-6

To a solution of Intermediate 1-4 (710 mg, 2.40 mmol, 1 eq) in dioxane (5 mL) and H₂O (1 mL) was added Intermediate 1-5 (573.19 mg, 2.40 mmol, 1 eq), K₃PO₄ (1.53 g, 7.19 mmol, 3 eq) and Pd(dtbpf)Cl₂ (156.29 mg, 239.80 μmol, 0.1 eq) at 25° C., the mixture was degassed and purged with N₂ 3 times, and then the mixture was stirred at 80° C. for 1 hr under N₂ atmosphere. The reaction mixture was diluted with H₂O (5 mL) and extracted with DCM (5 mL×3), the combined organic layers were dried over Na₂SO₄, filtered and concentrated to give a residue. The residue was purified by column chromatography (SiO₂, PE/EA=1/0 to 1/1) and concentrated to give Intermediate 1-6 (710 mg, 1.73 mmol, 72.15% yield) as a brown solid. LCMS (Method D): Rt=0.470 min, [M+H]⁺=411.1.

### Step 4: Synthesis of Intermediate 1-7

To a solution of Intermediate 1-6 (600 mg, 1.46 mmol, 1 eq) in MeOH (2 mL), THF (2 mL) and H₂O (2 mL) was added LiOH·H₂O (184.06 mg, 4.39 mmol, 3 eq). The mixture was stirred at 25° C. for 1 hr. The mixture was diluted with H₂O (5 mL) and HCl (1M) was added to adjust the pH to 4. The mixture was extracted with EA (5 mL*2). The combined organic layers were dried over Na₂SO₄, filtered and concentrated under reduced pressure to give Intermediate 1-7 (480 mg, 1.21 mmol, 82.83% yield, 100% purity) as a yellow solid. The product was used in the next step without further purification. LCMS (Method D): Rt=0.454 min, [M+H]⁺=397.0. ¹H NMR (400 MHz, METHANOL-d4) δ=9.27-9.22 (m, 1H), 8.55-8.48 (m, 1H), 7.77-7.69 (m, 2H), 7.60-7.54 (m, 1H), 7.53-7.47 (m, 1H), 3.69-3.58 (m, 2H), 1.57 (s, 9H). ¹⁹F NMR (376 MHz, METHANOL-d4) δ=67.377.

### Step 5: Synthesis of Intermediate 1-9

To a solution of Intermediate 1-7 (257.62 mg, 649.98 μmol, 1 eq) in DMF (5 mL) was added EDCI (373.80 mg, 1.95 mmol, 3 eq), HOAt (88.47 mg, 649.98 μmol, 90.92 μL, 1 eq), NMM (328.72 mg, 3.25 mmol, 357.30 μL, 5 eq) and Intermediate 1-8 (500.00 mg, 649.98 μmol, 1 eq, HCl salt). The mixture was stirred at 25° C. for 1 hr. The mixture was diluted with H₂O (10 mL) and extracted with EA (10 mL*2). The combined organic layers were dried over Na₂SO₄, filtered and concentrated under reduced pressure to give a residue. The residue was purified by reversed-phase HPLC (neutral condition), and the eluent was concentrated under reduced pressure to remove ACN and then lyophilized to give Intermediate 1-9 (400 mg, 357.11 μmol, 54.94% yield, 99.2% purity) as a yellow solid. LCMS (Method D): Rt: 0.587 min, (M+H)=1111.2.

### Step 6: Synthesis of I-841

To a solution of Intermediate 1-9 (60 mg, 54.00 μmol, 1 eq) in THF (0.8 mL) was added piperdine (86.22 mg, 1.01 mmol, 0.1 mL, 18.75 eq). The mixture was stirred at 25° C. for 0.5 hr. The mixture was concentrated in vacuo. The crude product was purified by reversed-phase column (0.10% FA condition). The eluent was concentrated under reduced pressure to remove ACN and then lyophilized to give I-841 (20.86 mg, 22.00 μmol, 40.74% yield, 98.6% purity, FA salt) as a white solid. LCMS (Method D): Rt: 0.439 min, [M+H]⁺=889.3. ¹H NMR (400 MHz, METHANOL-d4) δ=9.03 (s, 1H), 8.56-8.52 (m, 1H), 8.51-8.46 (m, 1H), 8.39-8.32 (m, 1H), 7.96-7.90 (m, 1H), 7.89-7.79 (m, 2H), 7.71-7.64 (m, 2H), 7.56-7.41 (m, 3H), 7.36-7.30 (m, 1H), 7.18-7.09 (m, 1H), 4.40-4.33 (m, 2H), 3.78-3.55 (m, 14H), 3.52-3.45 (m, 2H), 3.36-3.32 (m, 1H), 3.26-3.21 (m, 1H), 3.02-2.93 (m, 2H), 1.54 (s, 9H). ¹⁹F NMR (376 MHz, METHANOL-d4) δ=−67.339, −120.622.

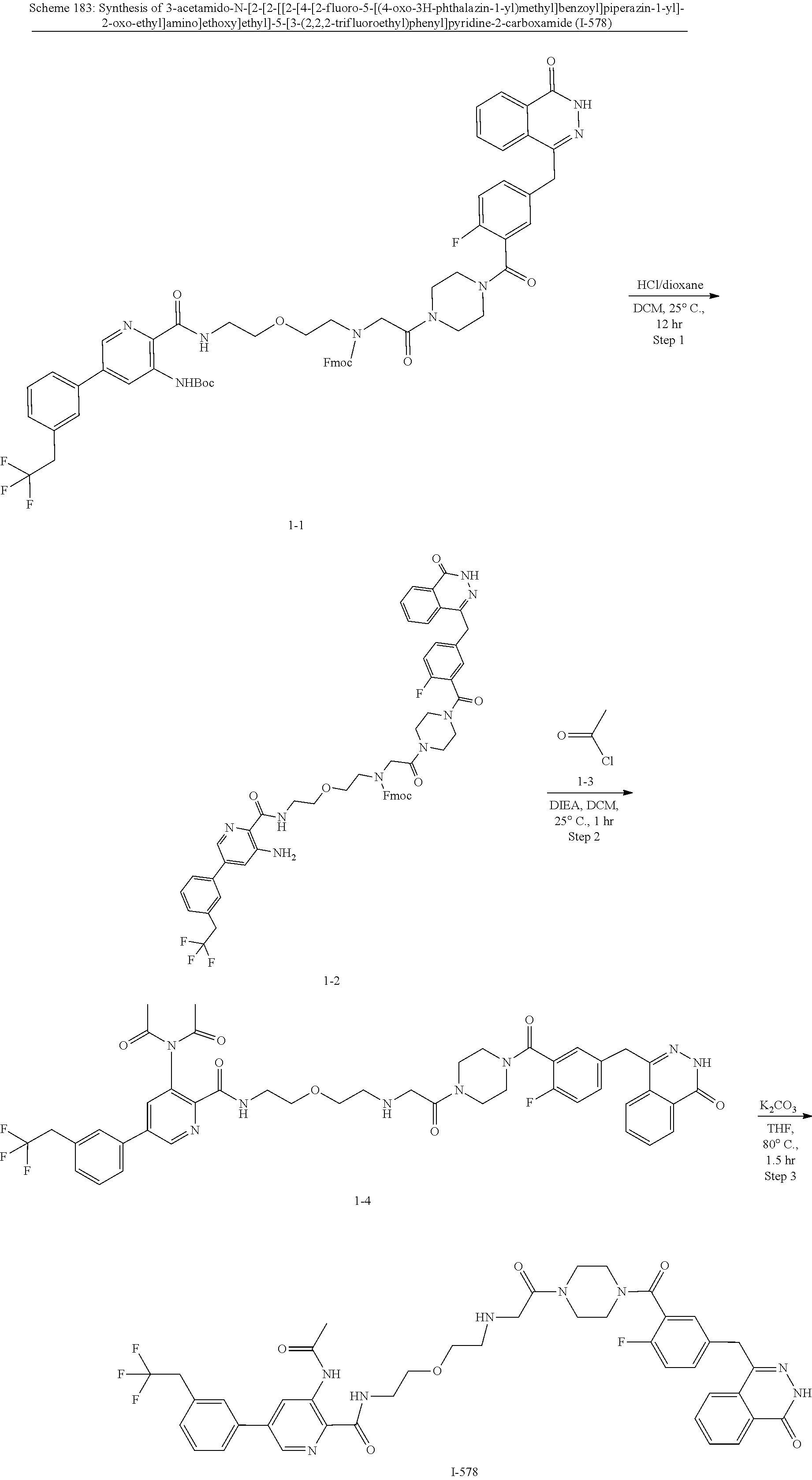

### Step 1: Synthesis of Intermediate 1-2

To a solution of Intermediate 1-1 (100 mg, 90.00 μmol, 1 eq) in CH₂Cl₂ (0.3 mL) was added HCl/dioxane (2 M, 1 mL). The mixture was stirred at 25° C. for 12 hr. The mixture was concentrated under reduced pressure to give Intermediate 1-2 (100 mg, crude, HCl salt) as a yellow oil, which was used in the next step without further purification. LCMS (Method D): Rt: 0.519 min, [M+H]⁺=1011.2.

### Step 2: Synthesis of Intermediate 1-4

To a solution of Intermediate 1-2 (100 mg, 95.47 μmol, 1 eq, HCl salt) in DCM (1 mL) was added DIEA (37.01 mg, 286.40 μmol, 49.88 μL, 3 eq) and Intermediate 1-3 (11.24 mg, 143.20 μmol, 10.18 μL, 1.5 eq). The mixture was stirred at 25° C. for 1 hr. The mixture was diluted with H₂O (1 mL) and extracted with CH₂Cl₂ (1 mL*2). The combined organic layers were dried over Na₂SO₄, filtered and concentrated under reduced pressure to give Intermediate 1-4 (110 mg, crude) as a yellow oil, which was used in the next step without further purification. LCMS (Method D): Rt: 0.499 min, [M+H]⁺=1095.2.

### Step 3: Synthesis of I-578

To a solution of Intermediate 1-4 (110 mg, 100.45 μmol, 1 eq) in THF (1 mL) was added K₂CO₃ (41.65 mg, 301.34 μmol, 3 eq). The mixture was stirred at 80° C. for 1.5 hr. The mixture was filtered. The filtrate was purified by reversed-phase HPLC (0.1% FA condition). The eluent was concentrated under reduced pressure to remove ACN and then lyophilized to give I-578 (35.65 mg, 39.60 μmol, 39.42% yield, 97.4% purity, FA salt) as a yellow solid. LCMS (Method D): Rt=0.390 min, [M+H]⁺=831.2. ¹H NMR (400 MHz, METHANOL-d4) δ=9.26 (s, 1H), 8.59-8.54 (m, 1H), 8.51 (s, 1H), 8.39-8.33 (m, 1H), 7.96-7.91 (m, 1H), 7.88-7.79 (m, 2H), 7.66 (s, 2H), 7.54-7.41 (m, 3H), 7.33 (d, J=6.0 Hz, 1H), 7.18-7.09 (m, 1H), 4.39-4.33 (m, 2H), 3.82 (s, 1H), 3.76-3.62 (m, 11H), 3.61-3.52 (m, 2H), 3.50-3.44 (m, 2H), 3.28-3.21 (m, 2H), 3.08-3.00 (m, 2H), 2.25 (s, 3H). ¹⁹F NMR (376 MHz, METHANOL-d4) δ=−67.347, −120.674.

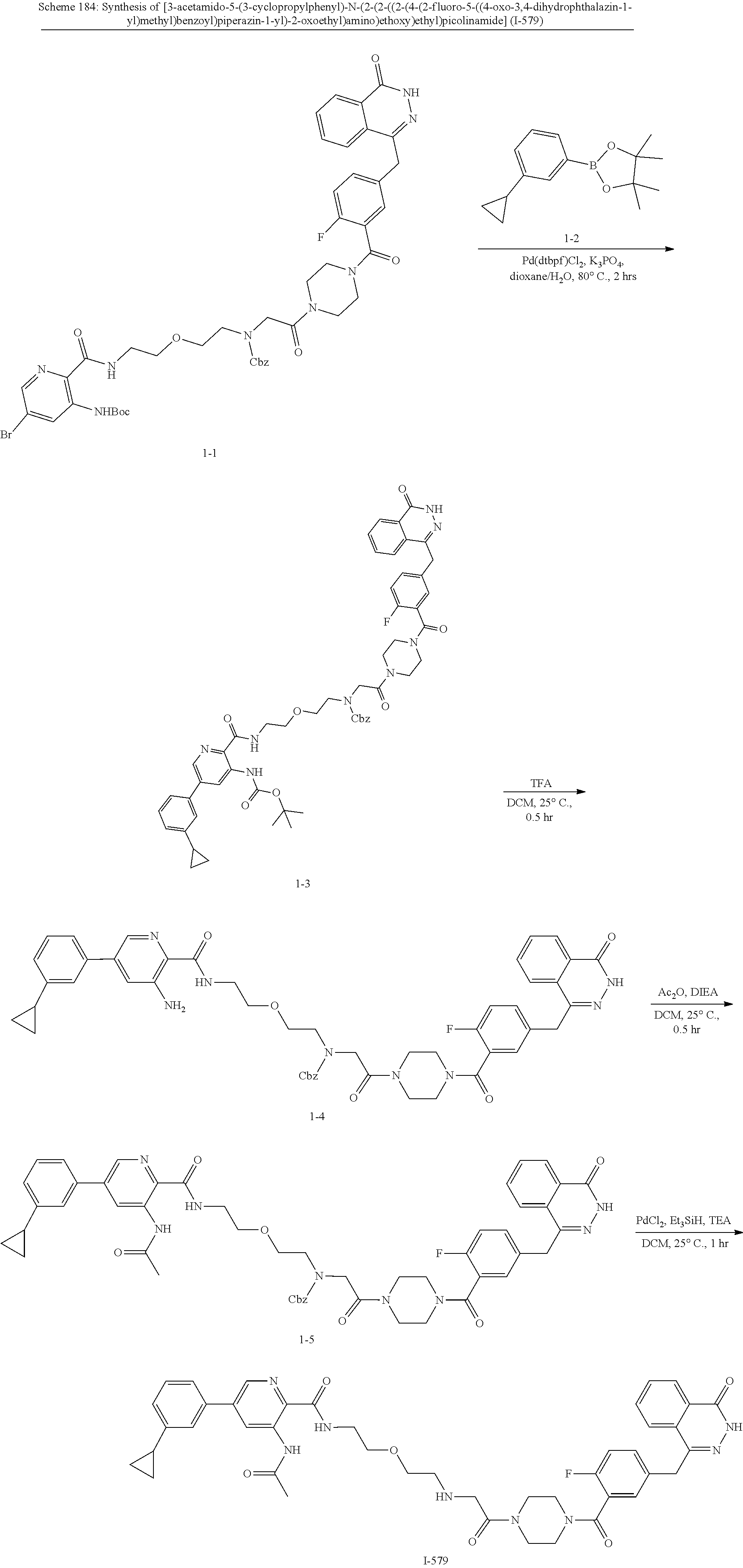

### Step 1: Synthesis of Intermediate 1-3

A mixture of intermediate 1-1 (500 mg, 529.77 μmol, 1 eq), intermediate 1-2 (142.27 mg, 582.74 mol, 1.1 eq), K₃PO₄ (337.35 mg, 1.59 mmol, 3 eq), and ditert-butyl(cyclopenta-1,4-dien-1-yl)phosphane;dichloropalladium;iron (34.53 mg, 52.98 μmol, 0.1 eq) in dioxane (5 mL) and H₂O (0.5 mL) was degassed and purged with N₂ 3 times, and then the mixture was stirred at 80° C. for 1 hr under N₂ atmosphere. The reaction mixture was diluted with H₂O (10 mL) and extracted with EA (10 mL*3). The combined organic layers were dried over Na₂SO₄, filtered and concentrated under reduced pressure to give a residue. The residue was purified by flash silica gel chromatography (ISCO; 20 g SepaFlash Silica Flash Column, Eluent of 0˜5% Methanol/Ethyl acetate gradient @60 mL/min), the eluent was concentrated under reduced pressure to give a residue. Intermediate 1-3 (440 mg, 425.50 μmol, 80.32% yield, 94.875% purity) was obtained as a yellow oil. LCMS (Method D): Retention time: 0.564 min, [M+H]⁺=981.3. ¹H NMR (400 MHz, METHANOL-d₄) δ=8.99-8.87 (m, 1H), 8.47-8.30 (m, 2H), 7.95-7.79 (m, 3H), 7.49-7.38 (m, 2H), 7.37-7.26 (m, 6H), 7.22 (d, J=4.8 Hz, 2H), 7.13 (s, 1H), 7.07-6.92 (m, 1H), 5.12 (s, 1H), 4.99 (d, J=7.2 Hz, 1H), 4.33 (d, J=18.8 Hz, 2H), 4.27-4.18 (m, 2H), 3.72 (s, 1H), 3.68-3.60 (m, 5H), 3.58-3.48 (m, 7H), 3.38 (s, 1H), 3.19 (d, J=11.6 Hz, 1H), 3.11-2.99 (m, 1H), 2.03-1.94 (m, 1H), 1.53 (s, 9H), 1.00 (s, 2H), 0.74 (s, 2H).

### Step 2: Synthesis of Intermediate 1-4.

To a solution of intermediate 1-3 (200 mg, 203.86 μmol, 1 eq) in DCM (2 mL) was added TFA (0.4 mL). The mixture was stirred at 25° C. for 0.5 hour. The reaction mixture was concentrated under reduced pressure to give a residue and the residue was washed with PE (3 mL*3). The crude product was used in the next step without further purification. Intermediate 1-4 (200 mg, 191.78 μmol, 94.07% yield, 95.407% purity, TFA salt) was obtained as a yellow oil. LCMS (Method D): Retention time: 0.489 min, [M+H]⁺=881.3. ¹H NMR (400 MHz, METHANOL-d₄) δ=8.36 (d, J=7.2 Hz, 1H), 7.93-7.77 (m, 3H), 7.51-7.19 (m, 12H), 7.15-7.00 (m, 2H), 5.13-4.98 (m, 2H), 4.38-4.24 (m, 4H), 3.68-3.48 (m, 12H), 3.22-3.11 (m, 2H), 3.02-2.83 (m, 2H), 2.02-1.91 (m, 1H), 1.00 (d, J=8.4 Hz, 2H), 0.73 (d, J=1.2 Hz, 2H). ¹⁹F NMR (376 MHz, METHANOL-d₄) δ=−77.479, −120.584.

### Step 3: Synthesis of Intermediate 1-5.

To a solution of intermediate 1-4 (150 mg, 150.76 μmol, 1 eq, TFA salt) in DCM (2 mL) was added DIEA (58.45 mg, 452.27 μmol, 78.78 μL, 3 eq) and acetyl chloride (11.83 mg, 150.76 μmol, 10.72 L, 1 eq). The mixture was stirred at 25° C. for 0.5 hr. The reaction mixture was diluted with H₂O (5 mL) and extracted with DCM (10 mL*3). The combined organic layers were concentrated under reduced pressure to give a residue. The crude product was purified by reversed-phase HPLC (neutral condition), the eluent was concentrated and lyophilized to give Intermediate 1-5 (130 mg, 140.85 μmol, 93.43% yield, 100% purity) as a yellow solid. LCMS (Method D): Retention time: 0.505 min, [M+H]⁺=923.5. ¹H NMR (400 MHz, METHANOL-d₄) δ=9.25-9.10 (m, 1H), 8.54-8.48 (m, 1H), 8.35 (d, J=6.8 Hz, 1H), 7.96-7.77 (m, 3H), 7.47-7.21 (m, 11H), 7.13 (d, J=7.2 Hz, 1H), 5.16-5.00 (m, 2H), 4.38-4.20 (m, 4H), 3.73-3.51 (m, 12H), 3.48 (s, 2H), 3.27-3.00 (m, 3H), 2.21 (d, J=12.8 Hz, 3H), 1.00 (s, 2H), 0.74 (s, 2H). ¹⁹F NMR (376 MHz, METHANOL-d₄) δ=−120.657.

### Step 4: Synthesis of I-579

A mixture of intermediate 1-5 (80 mg, 86.67 μmol, 1 eq), PdCl₂ (4.61 mg, 26.00 μmol, 0.3 eq), and TEA (17.54 mg, 173.35 μmol, 24.13 μL, 2 eq) in DCM (0.5 mL) was degassed and purged with N₂ 3 times, and then a solution of Et₃SiH (40.31 mg, 346.70 μmol, 55.37 μL, 4 eq) in DCM (0.5 mL) was added into the system at 10° C. The mixture was stirred at 10-25° C. for 1 hr under N₂ atmosphere. The reaction mixture was filtered and concentrated under reduced pressure to give a residue. The residue was purified by reversed-phase HPLC (FA condition), the eluent was concentrated and lyophilized to give I-579 (15.64 mg, 17.80 μmol, 20.54% yield, 95.024% purity, FA salt) as a white solid. LCMS (Method D): Retention time: 0.412 min, (M+H)=789.4. ¹H NMR (400 MHz, METHANOL-d₄) δ=9.20 (s, 1H), 8.61-8.42 (m, 2H), 8.35 (d, J=7.8 Hz, 1H), 7.95-7.88 (m, 1H), 7.87-7.76 (m, 2H), 7.47 (d, J=2.8 Hz, 1H), 7.42-7.28 (m, 4H), 7.17-7.05 (m, 2H), 4.35 (s, 2H), 3.80-3.52 (m, 12H), 3.46 (d, J=4.0 Hz, 2H), 3.28-3.24 (m, 1H), 3.22 (s, 1H), 3.00 (s, 2H), 2.23 (s, 3H), 2.03-1.89 (m, 1H), 1.03-0.92 (m, 2H), 0.79-0.65 (m, 2H). ¹⁹F NMR (376 MHz, METHANOL-d₄) δ=−120.612.

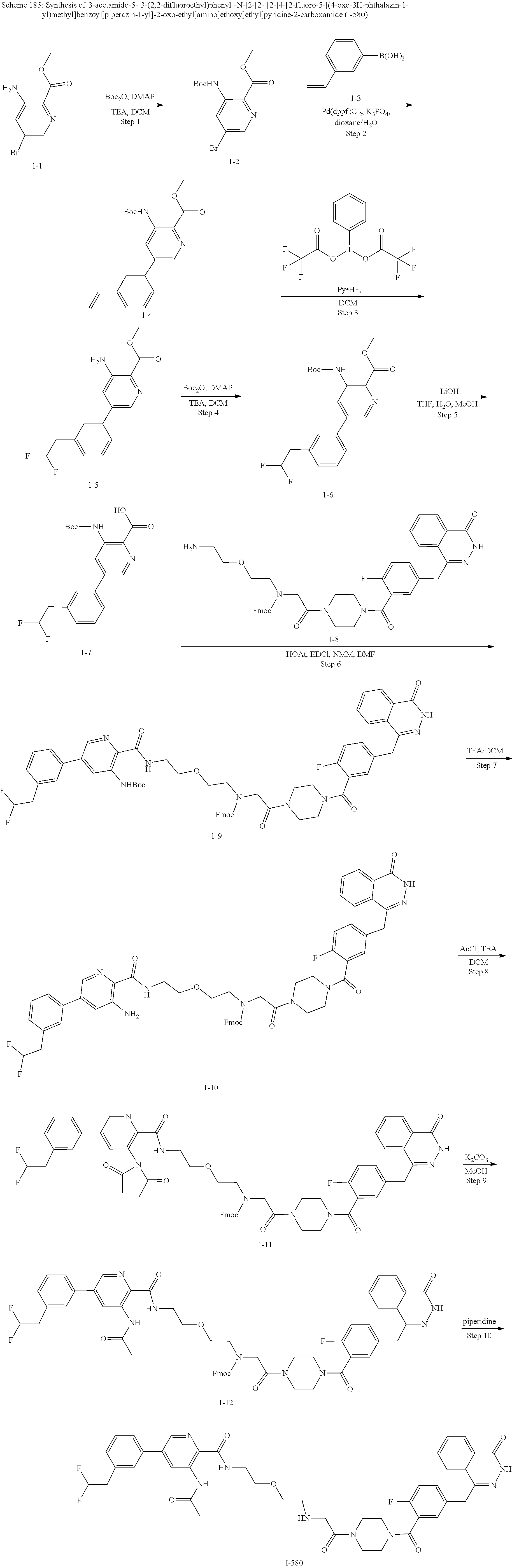

### Step 1: Synthesis of Intermediate 1-2.

To a mixture of Intermediate 1-1 (4.5 g, 19.48 mmol, 1 eq) in DCM (100 mL) was added Boc₂O (5.10 g, 23.37 mmol, 5.37 mL, 1.2 eq), TEA (3.94 g, 38.95 mmol, 5.42 mL, 2 eq), and DMAP (237.94 mg, 1.95 mmol, 0.1 eq), then the mixture was stirred at 20° C. for 12 hours. The reaction mixture was concentrated under reduced pressure to give a residue. The residue was purified by flash silica gel chromatography (ISCO; 50 g SepaFlash Silica Flash Column, Eluent of 0˜30% Ethylacetate/Petroleum ether gradient @80 mL/min). The eluent was concentrated under reduced pressure to give Intermediate 1-2 (4 g, 11.60 mmol, 59.54% yield, 96% purity) as a white solid. LCMS (Method D): Retention time: 0.461 min, (M+H)=274.9. ¹H NMR (400 MHz, CHLOROFORM-d₄) δ=10.21 (s, 1H), 9.14 (d, J=1.6 Hz, 1H), 8.37 (d, J=2.0 Hz, 1H), 4.02 (s, 3H), 1.54 (s, 9H).

### Step 2: Synthesis of Intermediate 1-4

To a mixture of Intermediate 1-2 (4 g, 12.08 mmol, 1 eq) and Intermediate 1-3 (2.68 g, 18.12 mmol, 1.5 eq) in dioxane (40 mL) and H₂O (8 mL) was added Pd(dppf)Cl₂ (883.80 mg, 1.21 mmol, 0.1 eq) and K₃PO₄ (7.69 g, 36.24 mmol, 3 eq), then the mixture was stirred at 80° C. for 1 hour under N₂. The reaction mixture was diluted with water (100 mL) and extracted with EA (40 mL*2). The combined organic layers were concentrated under reduced pressure to give a residue. The residue was purified by flash silica gel chromatography ((ISCO; 40 g SepaFlash Silica Flash Column, Eluent of 0˜30% Ethylacetate/Petroleum ether gradient @80 mL/min). The eluent was concentrated under reduced pressure to give Intermediate 1-4 (3.5 g, 9.88 mmol, 81.76% yield) as a yellow solid. LCMS (Method D): Retention time: 0.523 min, [M+H]⁺=355.1.

### Step 3: Synthesis of Intermediate 1-5.

To a mixture of Intermediate 1-5 (1 g, 2.82 mmol, 1 eq) in DCM (200 mL) was added [phenyl-(2,2,2-trifluoroacetyl)oxy-iodanyl]2,2,2-trifluoroacetate (1.33 g, 3.10 mmol, 1.1 eq) and pyridine;hydrofluoride (5.59 g, 39.50 mmol, 5.08 mL, 70% purity, 14 eq), then the mixture was stirred at 20° C. for 1 hour. To the reaction mixture was added a NaHCO₃ solution (about 100 mL) to adjust pH to 7-8. The mixture was then extracted with DCM (40 mL*3). The combined organic layers were concentrated under reduced pressure to give a residue. The crude product was purified by reversed-phase HPLC (0.1% FA condition). The eluent was concentrated under reduced pressure to remove ACN and then lyophilized to give Intermediate 1-5 (800 mg, 2.65 mmol, 31.36% yield, 97% purity) as a yellow solid. LCMS (Method D): Retention time: 0.326 min, [M+H]⁺=293.0. ¹H NMR (400 MHz, CHLOROFORM-d₄) δ=8.28 (d, J=2.0 Hz, 1H), 7.54-7.50 (m, 1H), 7.49-7.44 (m, 2H), 7.33 (d, J=7.6 Hz, 1H), 7.21 (d, J=2.0 Hz, 1H), 6.12-5.84 (m, 1H), 4.00 (s, 3H), 3.27-3.18 (m, 2H). ¹⁹F NMR (376 MHz, METHANOL-d₄) δ=−114.961.

### Step 4: Synthesis of Intermediate 1-6.

To a mixture of Intermediate 1-5 (600 mg, 2.05 mmol, 1 eq) in DCM (6 mL) was added Boc₂O (672.03 mg, 3.08 mmol, 707.40 μL, 1.5 eq), TEA (623.17 mg, 6.16 mmol, 857.18 μL, 3 eq) and DMAP (25.08 mg, 205.28 μmol, 0.1 eq), then the mixture was stirred at 20° C. for 1 hour. The reaction mixture was concentrated under reduced pressure to give a residue. The residue was purified by flash silica gel chromatography (ISCO; 20 g SepaFlash Silica Flash Column, Eluent of 0˜30% Ethyl acetate/Petroleum ether gradient @40 mL/min). The eluent was concentrated under reduced pressure to give Intermediate 1-6 (700 mg, 1.73 mmol, 84.29% yield, 97% purity) as a white solid. LCMS (Method D): Retention time: 0.503 min, [M+H]⁺=393.1.

### Step 5: Synthesis of Intermediate 1-7.

To a mixture of Intermediate 1-6 (640 mg, 1.63 mmol, 1 eq) in THF (4 mL), MeOH (2 mL) and H₂O (1 mL) was added LiOH·H₂O (136.89 mg, 3.26 mmol, 2 eq), then the mixture was stirred at 20° C. for 16 hours. The reaction mixture was concentrated under reduced pressure to remove THF, and then 1 N HCl was added (about 5 mL) to adjust pH to 3-4, and then the mixture was extracted with EA (10 mL*3). The combined organic layers were concentrated under reduced pressure to give Intermediate 1-7 (580 mg, 1.53 mmol, 93.98% yield) as a white solid. LCMS (Method D): Retention time: 0.436 min, [M+H]⁺=323.0.

### Step 6: Synthesis of Intermediate 1-9.

To a mixture of Intermediate 1-7 (100 mg, 264.29 μmol, 1 eq) and Intermediate 1-8 (203.31 mg, 264.29 μmol, 1 eq, HCl salt) in DMF (2 mL) was added HOAt (35.97 mg, 264.29 μmol, 36.97 μL, 1 eq), EDCI (151.99 mg, 792.88 μmol, 3 eq), and NMM (133.67 mg, 1.32 mmol, 145.29 μL, 5 eq), then the mixture was stirred at 20° C. for 1 hour. The reaction mixture was diluted with water (80 mL) and extracted with (30 mL*2). The combined organic layers were concentrated under reduced pressure to give Intermediate 1-9 (120 mg, crude) as a white solid. LCMS (Method D): Retention time: 0.576 min, [M+H]⁺=1093.2.

### Step 7: Synthesis of Intermediate 1-10.

A mixture of Intermediate 1-9 (100 mg, 91.48 μmol, 1 eq) in TFA (0.5 mL) and DCM (0.5 mL) was stirred at 20° C. for 10 min. The reaction mixture was concentrated under reduced pressure to give Intermediate 1-10 (100 mg, 90.33 μmol, 98.74% yield, TFA salt) as a white solid. LCMS (Method D): Retention time: 0.553 min, [M+H]⁺=993.4.

### Step 8: Synthesis of Intermediate 1-11.

To a mixture of Intermediate 1-10 (80 mg, 80.56 μmol, 1 eq) in DCM (1 mL) was added TEA (24.46 mg, 241.68 μmol, 33.64 μL, 3 eq) and acetyl chloride (6.32 mg, 80.56 μmol, 5.73 μL, 1 eq), then the mixture was stirred at 0° C. for 5 min. The reaction mixture was quenched with water (15 mL) and extracted with (10 mL*3). The combined organic layers were concentrated under reduced pressure to give Intermediate 1-11 (70 mg, 64.99 μmol, 80.67% yield) as a white solid. LCMS (Method D): Retention time: 0.509 min, [M+H]⁺=1077.3.

### Step 9: Synthesis of Intermediate 1-12.

To a mixture of Intermediate 1-11 (70 mg, 64.99 μmol, 1 eq) in MeOH (2 mL) was added K₂CO₃ (26.95 mg, 194.97 μmol, 3 eq), then the mixture was stirred at 25° C. for 1 hour. The reaction mixture was filtered and concentrated under reduced pressure to give Intermediate 1-12 (60 mg, 57.97 μmol, 89.20% yield) as a white solid. LCMS (Method D): Retention time: 0.419 min, [M+H]⁺=813.3.

### Step 10: Synthesis of I-580

A mixture of intermediate 1-12 (40 mg, 38.64 μmol, 1 eq) in piperidine (0.1 mL) and THF (0.8 mL) was stirred at 20° C. for 30 min. The reaction mixture was concentrated under reduced pressure to give a residue. The crude product was purified by reversed-phase HPLC (0.1% FA condition). The eluent was concentrated under reduced pressure to remove ACN and then lyophilized to give I-580 (9.78 mg, 11.31 mol, 29.25% yield, 99.281% purity, FA salt) as a white solid. LCMS (Method D): Retention time: 0.365 min, [M+H]⁺=813.7. ¹H NMR (400 MHz, METHANOL-d₄) δ=9.29-9.20 (m, 1H), 8.61-8.52 (m, 1H), 8.50 (s, 1H), 8.36 (d, J=7.6 Hz, 1H), 7.96-7.90 (m, 1H), 7.88-7.79 (m, 2H), 7.63-7.55 (m, 2H), 7.52-7.44 (m, 2H), 7.42-7.36 (m, 1H), 7.34-7.29 (m, 1H), 7.17-7.13 (m, 1H), 6.25-5.88 (m, 1H), 4.36 (d, J=3.6 Hz, 2H), 3.84 (s, 1H), 3.78-3.60 (m, 11H), 3.46 (s, 2H), 3.25-3.12 (m, 4H), 3.05 (d, J=4.8 Hz, 2H), 2.25 (s, 3H). ¹⁹F NMR (376 MHz, METHANOL-d₄) δ=−116.951, −120.662,

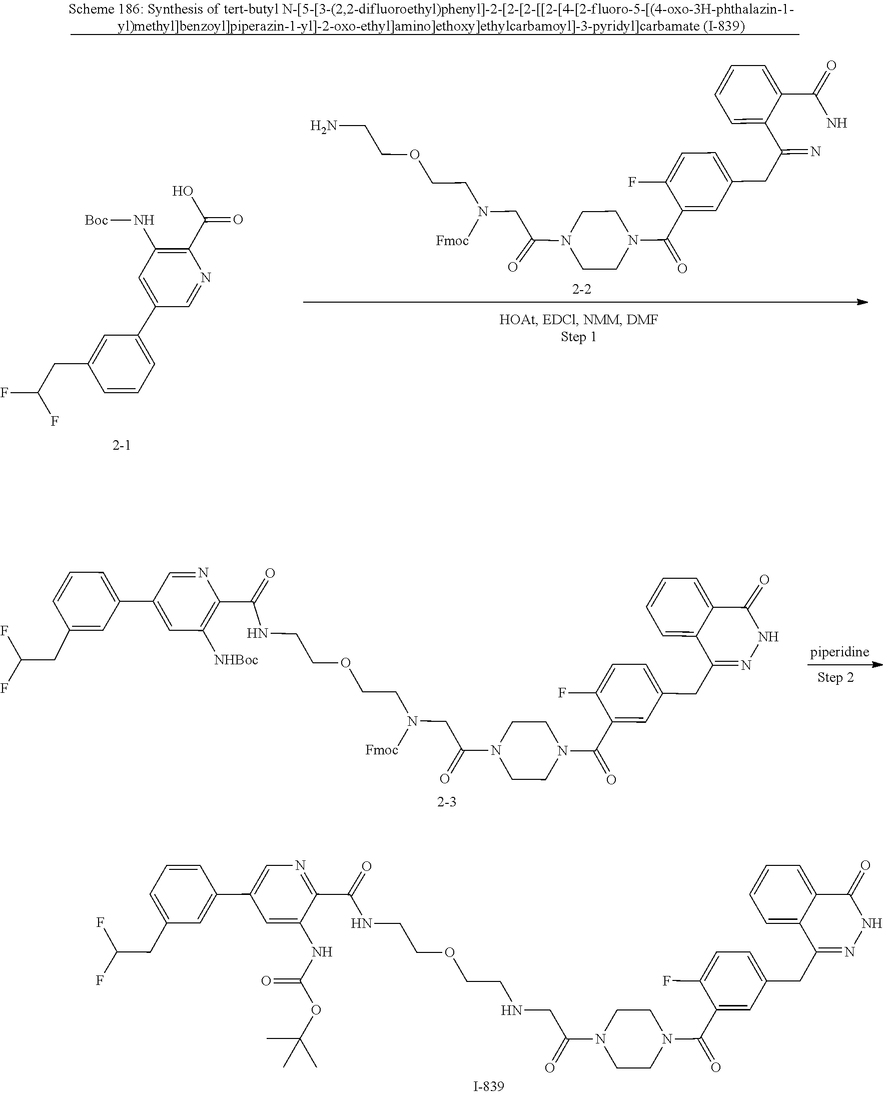

### Step 1: Synthesis of Intermediate 2-3.

To a mixture of Intermediate 2-1 (30 mg, 79.29 μmol, 1 eq) and Intermediate 2-2 (60.99 mg, 79.29 μmol, 1 eq, HCl salt) in DMF (1 mL) was added HOAt (10.79 mg, 79.29 μmol, 11.09 μL, 1 eq), EDCI (45.60 mg, 237.86 gmol, 3 eq), and NMM (40.10 mg, 396.44 gmol, 43.59 μL, 5 eq), then the mixture was stirred at 20° C. for 1 hour. The reaction mixture was diluted with water (50 mL) and extracted with EA (10 mL*3). The combined organic layers were concentrated under reduced pressure to give Intermediate 2-3 (50 mg, crude) as a white solid. LCMS (Method D): Retention time: 0.581 min, [M+H]⁺=1093.2.

### Step 2: Synthesis of I-839.

A mixture of Intermediate 2-3 (40 mg, 36.59 μmol, 1 eq) in piperidine (0.1 mL) and THF (0.8 mL) was stirred at 20° C. for 1 hour. The reaction mixture was concentrated under reduced pressure to give a residue. The crude product was purified by reversed-phase HPLC (0.1% FA condition). The eluent was concentrated under reduced pressure to remove ACN and then lyophilized to give I-839 (13.86 mg, 15.76 mol, 43.08% yield, 99.059% purity) as a white solid. LCMS (Method D): Retention time: 0.446 min, [M+H]⁺=871.7. ¹H NMR (400 MHz, METHANOL-d₄) δ=9.01 (d, J=3.2 Hz, 1H), 8.48 (d, J=9.6 Hz, 1H), 8.36 (d, J=7.2 Hz, 1H), 7.98-7.90 (m, 1H), 7.88-7.79 (m, 2H), 7.66-7.58 (m, 2H), 7.48 (d, J=6.4 Hz, 2H), 7.44-7.36 (m, 1H), 7.35-7.30 (m, 1H), 7.15-7.11 (m, 1H), 6.27-5.89 (m, 1H), 4.36 (d, J=6.0 Hz, 2H), 3.87 (s, 1H), 3.81-3.60 (m, 11H), 3.53-3.43 (m, 3H), 3.41-3.34 (m, 2H), 3.29-3.13 (m, 5H), 3.11-3.02 (m, 2H), 1.54 (s, 9H). ¹⁹F NMR (376 MHz, METHANOL-d₄) δ=−116.942, −120.662.

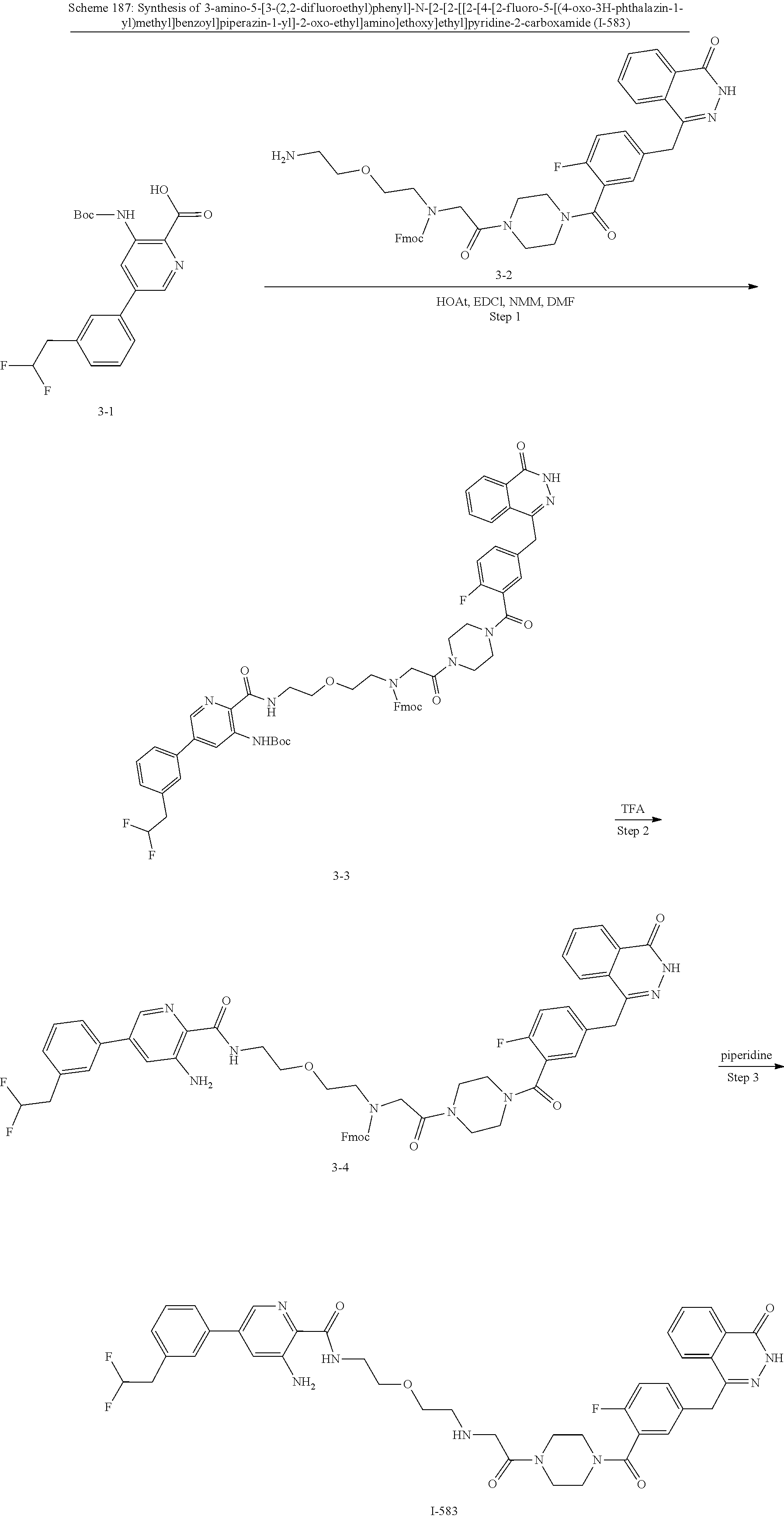

### Step 1: Synthesis of Intermediate 3-3.

To a mixture of Intermediate 3-1 (50 mg, 132.15 μmol, 1 eq) and Intermediate 3-2 (101.65 mg, 132.15 μmol, 1 eq, HCl salt) in DMF (1 mL) was added HOAt (17.99 mg, 132.15 μmol, 18.49 μL, 1 eq), EDCI (76.00 mg, 396.44 μmol, 3 eq), and NMM (66.83 mg, 660.73 μmol, 72.64 μL, 5 eq), then the mixture was stirred at 20° C. for 1 hour. The reaction mixture was diluted with water (50 mL) and extracted with EA (10 mL*3). The combined organic layers were concentrated under reduced pressure to give Intermediate 3-3 (80 mg, crude) as a white solid. LCMS (Method D): Retention time: 0.578 min, [M+H]⁺=1093.2.

### Step 2: Synthesis of Intermediate 3-4.

A mixture of Intermediate 3-3 (60 mg, 54.89 μmol, 1 eq) in TFA (0.1 mL) and DCM (1 mL) was stirred at 20° C. for 1 hour. The reaction mixture was concentrated under reduced pressure to give Intermediate 3-4 (50 mg, crude, TFA) as a white solid. LCMS (Method D): Retention time: 0.509 min, [M+H]⁺=993.2.

### Step 3: Synthesis of I-583

A mixture of intermediate 3-4 (40 mg, 40.28 μmol, 1 eq) in piperidine (0.1 mL) and THF (0.8 mL) was stirred at 20° C. for 1 hour. The reaction mixture was concentrated under reduced pressure to give a residue. The crude product was purified by reversed-phase HPLC (0.1% FA condition). The eluent was concentrated under reduced pressure to remove ACN and then lyophilized to give I-583 (14.58 mg, 18.92 mol, 46.96% yield, 100% purity) as a white solid. LCMS (Method D): Retention time: 0.361 min, [M+H]⁺=771.5. ¹H NMR (400 MHz, METHANOL-d₄) δ=8.39-8.33 (m, 1H), 8.06 (d, J=14.4 Hz, 1H), 7.94-7.90 (m, 1H), 7.89-7.78 (m, 2H), 7.57-7.50 (m, 2H), 7.48-7.39 (m, 2H), 7.33 (d, J=4.4 Hz, 3H), 7.15-7.12 (m, 1H), 6.26-5.86 (m, 1H), 4.36 (d, J=2.8 Hz, 2H), 3.65 (s, 7H), 3.60-3.55 (m, 3H), 3.52-3.45 (m, 3H), 3.37-3.33 (m, 1H), 3.28-3.18 (m, 4H), 2.86-2.79 (m, 2H). ¹⁹F NMR (376 MHz, METHANOL-d₄) δ=−116.876, δ=−120.652.

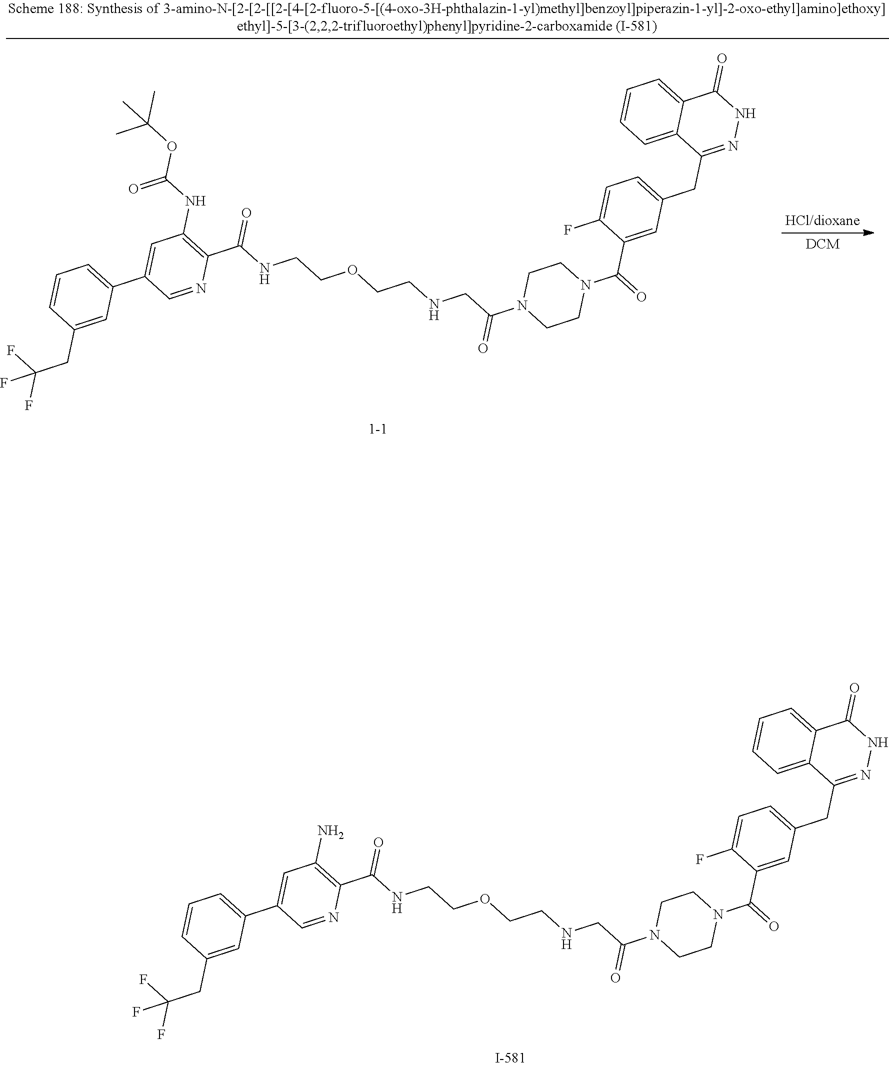 [SMILES: CC(C)(C)OC(=O)Nc1cc(-c2cccc(CC(F)(F)F)c2)cnc1C(=O)NCCOCCNCC(=O)N1CCN(C(=O)c2cc(Cc3n[nH]c(=O)c4ccccc34)ccc2F)CC1 ; Nc1cc(-c2cccc(CC(F)(F)F)c2)cnc1C(=O)NCCOCCNCC(=O)N1CCN(C(=O)c2cc(Cc3n[nH]c(=O)c4ccccc34)ccc2F)CC1]

To a solution of intermediate 1-1 (50 mg, 56.25 gmol, 1 eq) in CH₂Cl₂ (0.2 mL) and HCl/dioxane (2 M, 0.5 mL) was stirred at 25° C. for 1 hour. The mixture was concentrated in vacuum. The crude product was purified by reversed-phase HPLC (0.1% HCl condition). The eluent was concentrated under reduced pressure to remove ACN and then lyophilized to give I-581 (21.75 mg, 26.36 μmol, 46.86% yield, 100% purity, HCl salt) was obtained as a yellow solid. LCMS (Method D): Rt: 0.387 min, [M+H]⁺=789.3. ¹H NMR (400 MHz, METHANOL-d₄) δ=8.40-8.36 (m, 1H), 8.18-8.12 (m, 1H), 7.99-7.93 (m, 1H), 7.92-7.81 (m, 2H), 7.64 (s, 2H), 7.56-7.41 (m, 4H), 7.38-7.31 (m, 1H), 7.21-7.11 (m, 1H), 4.42-4.36 (m, 2H), 4.23-4.10 (m, 2H), 3.88-3.82 (m, 2H), 3.80-3.71 (m, 4H), 3.71-3.56 (m, 5H), 3.55-3.46 (m, 2H), 3.32-3.27 (m, 5H), 1.98 (s, 1H). ¹⁹F NMR (376 MHz, METHANOL-d₄) δ=−67.377, −120.637.

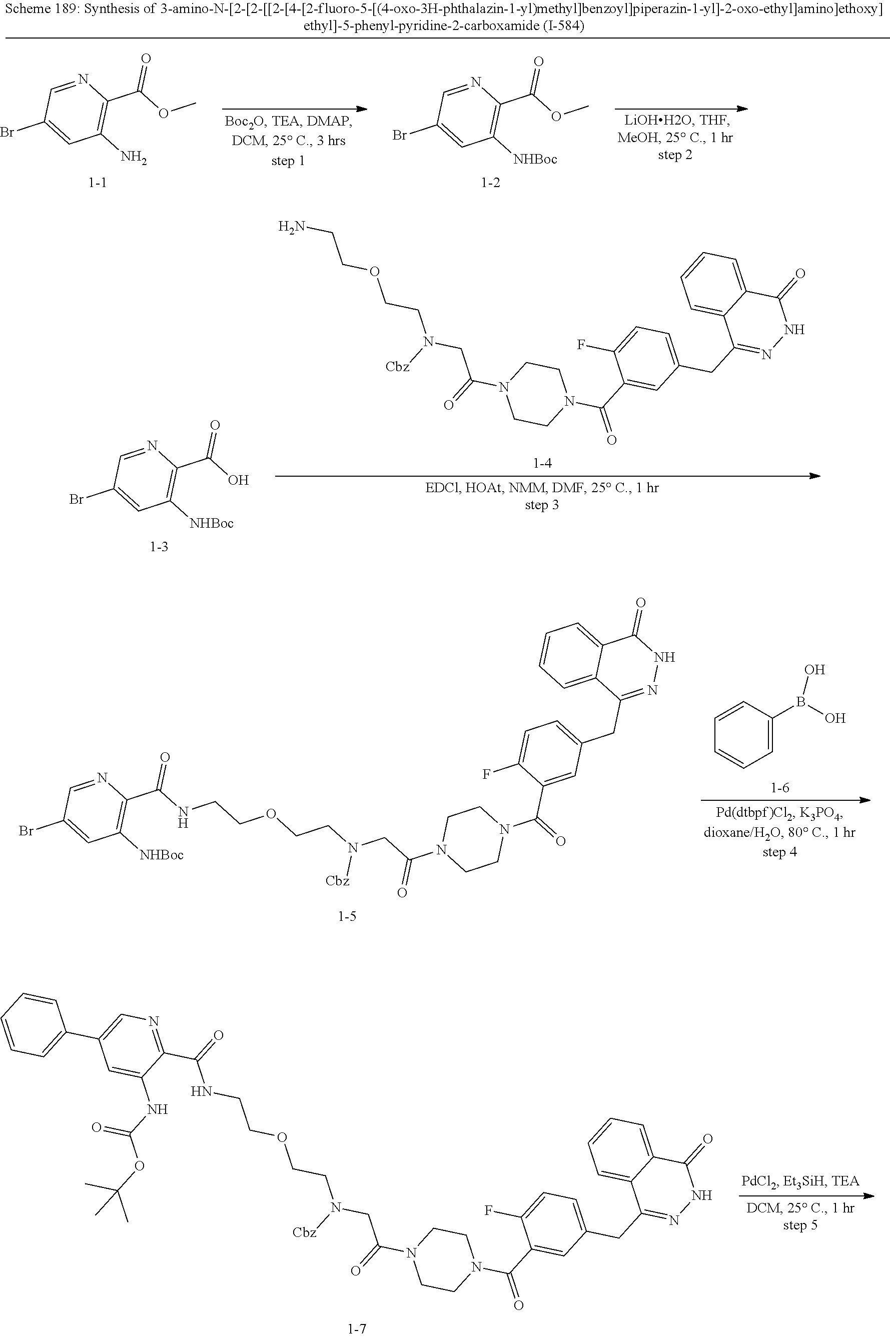

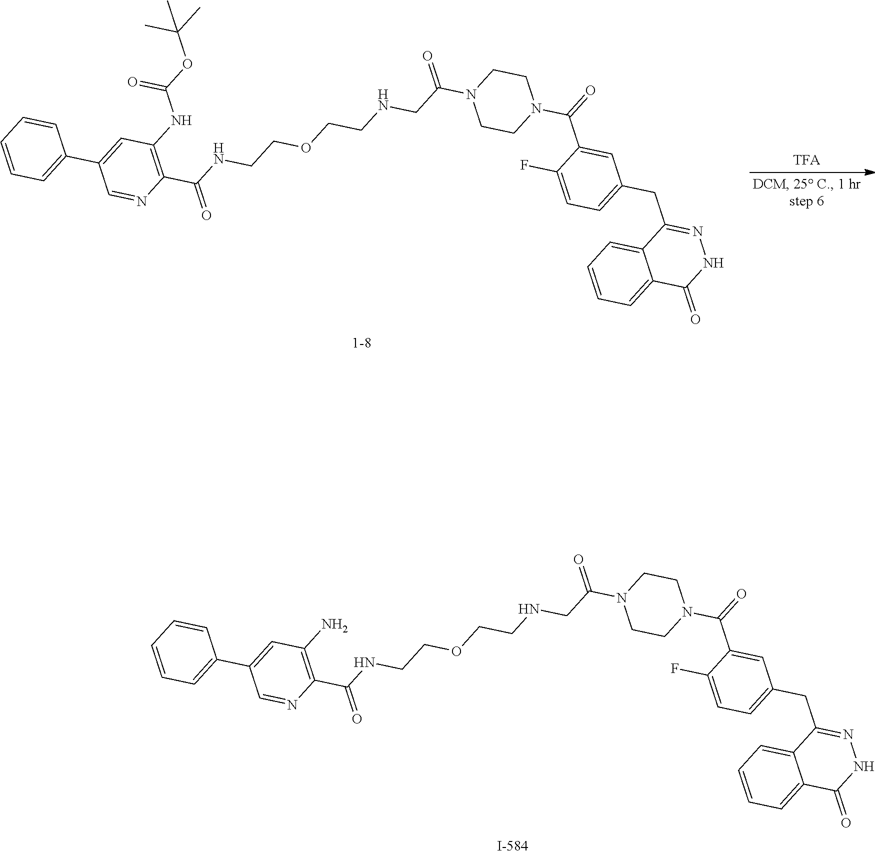 [SMILES: CC(C)(C)OC(=O)Nc1cc(-c2ccccc2)cnc1C(=O)NCCOCCNCC(=O)N1CCN(C(=O)c2cc(Cc3n[nH]c(=O)c4ccccc34)ccc2F)CC1 ; Nc1cc(-c2ccccc2)cnc1C(=O)NCCOCCNCC(=O)N1CCN(C(=O)c2cc(Cc3n[nH]c(=O)c4ccccc34)ccc2F)CC1]

### Step 1: Synthesis of Intermediate 1-2

To a solution of intermediate 1-1 (20 g, 86.56 mmol, 1 eq) in DCM (200 mL) was added Boc₂O (20.78 g, 95.22 mmol, 21.87 mL, 1.1 eq), TEA (26.28 g, 259.69 mmol, 36.15 mL, 3 eq) and DMAP (1.06 g, 8.66 mmol, 0.1 eq). The mixture was stirred at 25° C. for 3 hrs. The reaction mixture was diluted with H₂O (100 mL) and extracted with EA (100 mL*2). The combined organic layers were dried over Na₂SO₄, filtered and concentrated under reduced pressure to give a residue. The residue was purified by flash silica gel chromatography (ISCO; 220 g SepaFlash Silica Flash Column, Eluent of 0˜18% Ethylacetate/Petroleum ether gradient @100 mL/min). The eluent was concentrated under reduced pressure to give intermediate 1-2 (12.3 g, 36.98 mmol, 42.72% yield, 99.560% purity) as a white solid. LCMS (Method D): Retention time: 0.478 min, [M+H]⁺=331.0.

### Step 2: Synthesis of Intermediate 1-3.

To a solution of intermediate 1-2 (12.3 g, 37.14 mmol, 1 eq) in MeOH (40 mL), THF (40 mL) and H₂O (40 mL) was added LiOHH₂O (4.68 g, 111.43 mmol, 3 eq). The mixture was stirred at 25° C. for 1 hr. The reaction mixture was diluted with H₂O (50 mL) and added HCl (1M) to adjust pH to 4. The mixture was extracted with EA (50 mL*2). The combined organic layers were dried over Na₂SO₄, filtered and concentrated under reduced pressure to give intermediate 1-3 (11.57 g, 36.48 mmol, 98.23% yield, 100% purity) as a white solid was used in the next step without further purification. LCMS (Method D): Retention time: 0.422 min, [M-tBu+H]⁺=261.0. ¹H NMR (400 MHz, METHANOL-d₄) δ=9.09 (s, 1H), 8.33 (s, 1H), 1.55 (s, 9H).

### Step 3: Synthesis of Intermediate 1-5.

To a solution of Intermediate 1-3 (17.63 g, 25.88 mmol, 1 eq, HCl salt) in DMF (180 mL) was added Intermediate 1-4 (8.21 g, 25.88 mmol, 1 eq), EDCI (14.89 g, 77.65 mmol, 3 eq), HOAt (3.52 g, 25.88 mmol, 3.62 mL, 1 eq) and NMM (13.09 g, 129.41 mmol, 14.23 mL, 5 eq). The mixture was stirred at 25° C. for 1 hr. The reaction mixture was diluted with H₂O (150 mL) and extracted with EA (100 mL*2). The combined organic layers were washed with brine (50 mL*1) and dried over Na₂SO₄, filtered and concentrated under reduced pressure to give a residue. The residue was purified by flash silica gel chromatography (ISCO; 330 g SepaFlash Silica Flash Column, Eluent of 0˜10% Dichloromethane/Methanol ether gradient @150 mL/min). The eluent was concentrated under reduced pressure to give Intermediate 1-5 (14.1 g, 14.56 mmol, 56.26% yield, 97.469% purity) as a yellow solid. LCMS (Method D): Retention time: 0.525 min, [M+H]⁺=943.3. ¹H NMR (400 MHz, METHANOL-d₄) δ =9.02-8.84 (m, 1H), 8.35 (d, J=7.6 Hz, 1H), 8.27-8.08 (m, 1H), 7.96-7.90 (m, 1H), 7.88-7.77 (m, 2H), 7.53-7.43 (m, 1H), 7.43-7.21 (m, 6H), 7.20-7.11 (m, 1H), 5.14-4.96 (m, 2H), 4.37 (s, 2H), 4.25 (s, 1H), 4.16 (d, J=6.0 Hz, 1H), 3.81-3.70 (m, 1H), 3.67-3.45 (m, 12H), 3.45-3.37 (m, 1H), 3.30-3.25 (m, 1H), 3.21-3.10 (m, 1H), 1.50 (d, J=4.0 Hz, 9H). ¹⁹F NMR (376 MHz, METHANOL-d₄) δ=−120.437.

### Step 4: Synthesis of Intermediate 1-7.

To a solution of Intermediate 1-5 (200 mg, 211.91 μmol, 1 eq) in H₂O (0.4 mL), 1,4-dioxane (1 mL) was added Intermediate 1-6 (31.01 mg, 254.29 μmol, 1.2 eq), K₃PO₄ (134.94 mg, 635.72 μmol, 3 eq) and Pd(dtbpf)Cl₂ (13.81 mg, 21.19 μmol, 0.1 eq). The mixture was stirred at 80° C. for 1 hour under N₂. The reaction mixture was diluted with H₂O (1 mL) and extracted with EA (2 mL*2). The combined organic layers were dried over Na₂SO₄, filtered and concentrated under reduced pressure to give a residue. The residue was purified by flash silica gel chromatography (ISCO; 20 g SepaFlash Silica Flash Column, Eluent of 0˜100% Ethylacetate/Petroleum ether gradient @60 mL/min). The eluent was concentrated under reduced pressure to give Intermediate 1-7 (150 mg, 156.22 μmol, 73.72% yield, 98.002% purity) as a white solid. LCMS (Method D): Retention time: 0.546 min, [M+H]⁺=941.1.

### Step 5: Synthesis of Intermediate 1-8.

To a solution of intermediate 1-7 (150 mg, 159.40 gmol, 1 eq) in DCM (1.5 mL) was added PdCl₂ (8.48 mg, 47.82 μmol, 0.3 eq) and TEA (32.26 mg, 318.81 μmol, 44.37 μL, 2 eq). The reaction mixture was degassed with N₂. Then a solution of Et₃SiH (74.14 mg, 637.61 μmol, 101.84 μL, 4 eq) in DCM (1.5 mL) was added dropwise at 10° C. The mixture was stirred at 25° C. for 1 hr. The reaction mixture was diluted with a saturated Na₂CO₃ aqueous solution (2 mL) and extracted with EA (3 mL*2). The combined organic layers were dried over Na₂SO₄, filtered and concentrated under reduced pressure to give a residue. The crude product was purified by reversed-phase HPLC (neutral condition). The eluent was concentrated under reduced pressure to remove ACN and then lyophilized to give Intermediate 1-8 (120 mg, 147.55 μmol, 92.57% yield, 99.214% purity) as a white solid. LCMS (Method D): Retention time: 0.427 min, [M+H]⁺=807.2.

### Step 6: Synthesis of I-584

A solution of intermediate 1-8 (100 mg, 123.93 μmol, 1 eq) in DCM (1 mL) and TFA (0.2 mL) was stirred at 25° C. for 1 hr. The crude product was purified by prep-HPLC (column: CD04-Welch Utimate C18 150*25*7 um; mobile phase: [water (TFA)-ACN]; gradient: 22%-52% B over 10 min). The eluent was concentrated under reduced pressure to remove ACN and then lyophilized to give I-584 (46.43 mg, 56.37 mol, 45.49% yield, 99.659% purity, TFA salt) was obtained as a white solid. LCMS (Method D): Retention time: 0.354 min, [M+H]⁺=707.3. ¹H NMR (400 MHz, METHANOL-d₄) δ=8.41-8.33 (m, 1H), 8.11-8.03 (m, 1H), 8.11-8.03 (m, 1H), 7.97-7.91 (m, 1H), 7.89-7.79 (m, 2H), 7.65-7.57 (m, 2H), 7.53-7.27 (m, 6H), 7.20-7.09 (m, 1H), 4.37 (d, J=4.8 Hz, 2H), 4.18-4.06 (m, 2H), 3.86-3.79 (m, 2H), 3.78-3.69 (m, 4H), 3.68-3.60 (m, 3H), 3.52-3.43 (m, 2H), 3.30-3.25 (m, 5H). ¹⁹FNMR (376 MHz, METHANOL-d₄) δ=−77.314, −120.622.

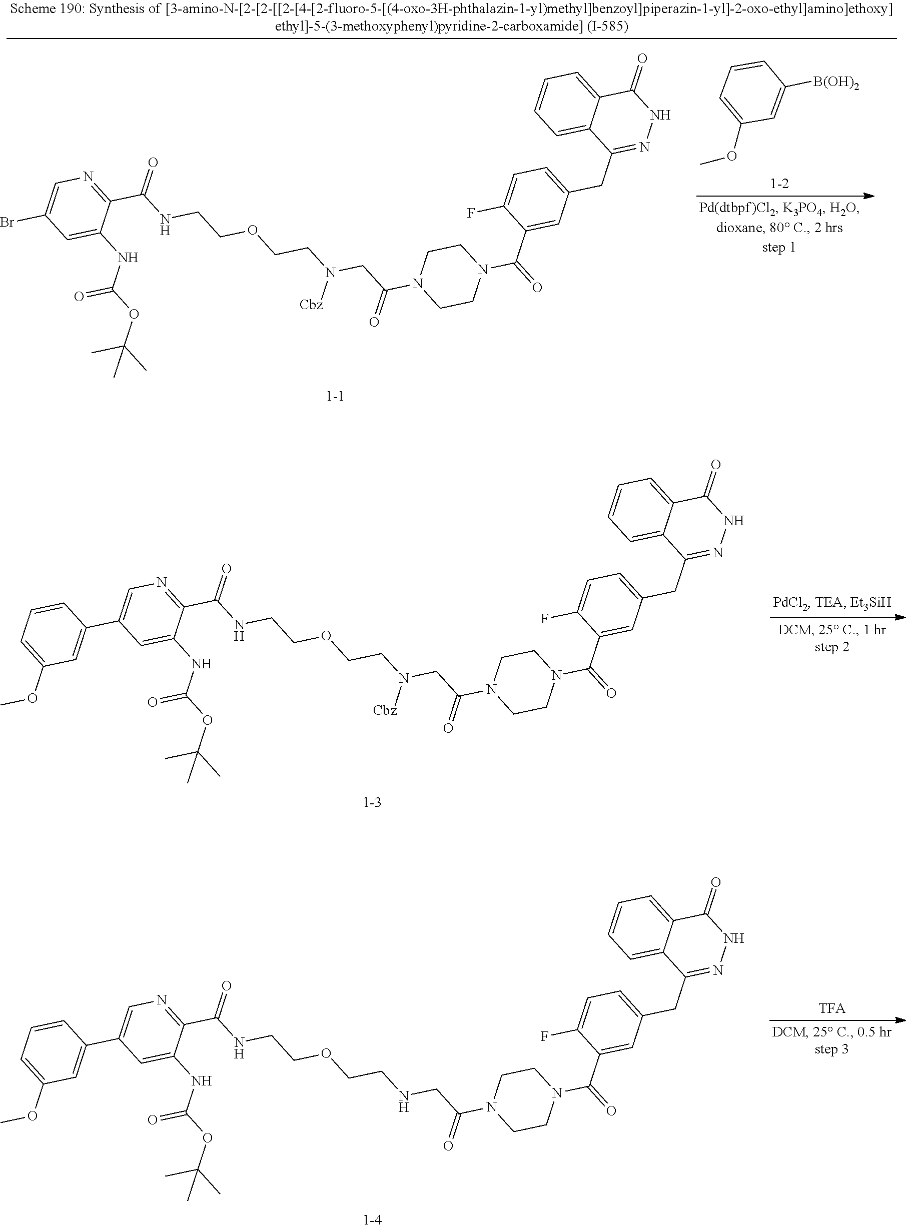 [SMILES: CC(C)(C)OC(=O)Nc1cc(Br)cnc1C(=O)NCCOCCN(CC(=O)N1CCN(C(=O)c2cc(Cc3n[nH]c(=O)c4ccccc34)ccc2F)CC1)C(=O)OCc1ccccc1 ; COc1cccc(B(O)O)c1 ; COc1cccc(-c2cnc(C(=O)NCCOCCN(CC(=O)N3CCN(C(=O)c4cc(Cc5n[nH]c(=O)c6ccccc56)ccc4F)CC3)C(=O)OCc3ccccc3)c(NC(=O)OC(C)(C)C)c2)c1 ; COc1cccc(-c2cnc(C(=O)NCCOCCNCC(=O)N3CCN(C(=O)c4cc(Cc5n[nH]c(=O)c6ccccc56)ccc4F)CC3)c(NC(=O)OC(C)(C)C)c2)c1]

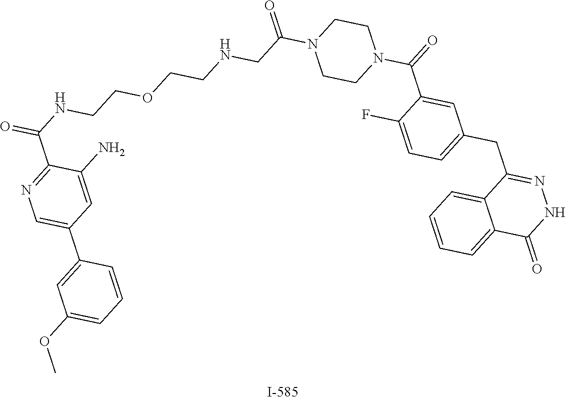 [SMILES: COc1cccc(-c2cnc(C(=O)NCCOCCNCC(=O)N3CCN(C(=O)c4cc(Cc5n[nH]c(=O)c6ccccc56)ccc4F)CC3)c(N)c2)c1]

### Step 1: Synthesis of Intermediate 1-3.

A mixture of Intermediate 1-1 (250 mg, 264.88 μmol, 1 eq), Intermediate 1-2 (40.25 mg, 264.88 mol, 1 eq), ditert-butyl(cyclopenta-1,4-dien-1-yl)phosphane;dichloropalladium;iron (17.26 mg, 26.49 mol, 0.1 eq), and tripotassium;phosphate (168.68 mg, 794.65 μmol, 3 eq) in dioxane (2.0 mL) and H₂O (0.4 mL) was degassed and purged with N₂ 3 times, and then the mixture was stirred at 80° C. for 2 hours under N₂ atmosphere. The reaction mixture was quenched by addition of H₂O (5 mL) at 25° C., and extracted with DCM (10 mL*3), dried over Na₂SO₄, filtered and concentrated under reduced pressure to give a residue. The crude product was purified by reversed-phase chromatography (neutral condition), the eluent was concentrated and lyophilized to give Intermediate 1-3 (250 mg, 256.58 μmol, 96.86% yield, 99.659% purity) as a white solid. LCMS (Method F): Retention time: 0.550 min, [M+H]⁺=971.2.

### Step 2: Synthesis of Intermediate 1-4.

To a solution of Intermediate 1-3 (80 mg, 82.39 μmol, 1 eq) in DCM (0.6 mL) was added PdCl₂ (7.30 mg, 41.19 μmol, 0.5 eq) and TEA (25.01 mg, 247.16 μmol, 34.40 μL, 3 eq). The reaction mixture was degassed with N₂. Then Et₃SiH (114.96 mg, 988.63 μmol, 157.91 μL, 12 eq) was added into the mixture. The mixture was stirred at 25° C. for 1 hr. The reaction mixture was quenched by addition of H₂O (3 mL) at 25° C., and extracted with DCM (3 mL*3), the combined organic phase was dried over Na₂SO₄, filtered and concentrated under reduced pressure to give a crude product. The crude product was used for the next step directly. Intermediate 1-4 (80 mg, crude) was obtained as a yellow solid. LCMS (Method D): Retention time: 0.424 min, [M+H]⁺=837.2.

### Step 3: Synthesis of I-585

To a solution of Intermediate 1-4 (40 mg, 47.80 μmol, 1 eq) in DCM (0.5 mL) was added TFA (153.50 mg, 1.35 mmol, 0.1 mL). The mixture was stirred at 25° C. for 0.5 hr. The mixture was dried with N₂ to give a crude product. The crude product was purified by prep-HPLC (column: CD04-Welch Utimate C18 150*25*7 um; mobile phase: [water(TFA)-ACN]; gradient: 28%-58% B over 10 min) and lyophilized to give I-585 (16.27 mg, 19.12 μmol, 40.01% yield, 100% purity, TFA salt) as a white solid. LCMS (Method D): Retention time: 0.336 min, [M+H]⁺=737.3. ¹H NMR (400 MHz, METHANOL-d₄) δ=8.37-8.08 (m, 1H), 8.12-8.00 (m, 1H), 7.96-7.90 (m, 1H), 7.89-7.80 (m, 2H), 7.49-7.36 (m, 1H), 7.34-7.28 (m, 3H), 7.20-7.10 (m, 3H), 7.03-6.94 (m, 1H), 4.36 (d, J=4.4 Hz, 2H), 4.17-4.06 (m, 2H), 3.86-3.80 (m, 5H), 3.76-3.69 (m, 4H), 3.67-3.60 (m, 3H), 3.52-3.48 (m, 1H), 3.44 (t, J=5.2 Hz, 1H), 3.28 (s, 5H). ¹⁹F NMR (376 MHz, METHANOL-d₄) δ=−77.209, −120.614.

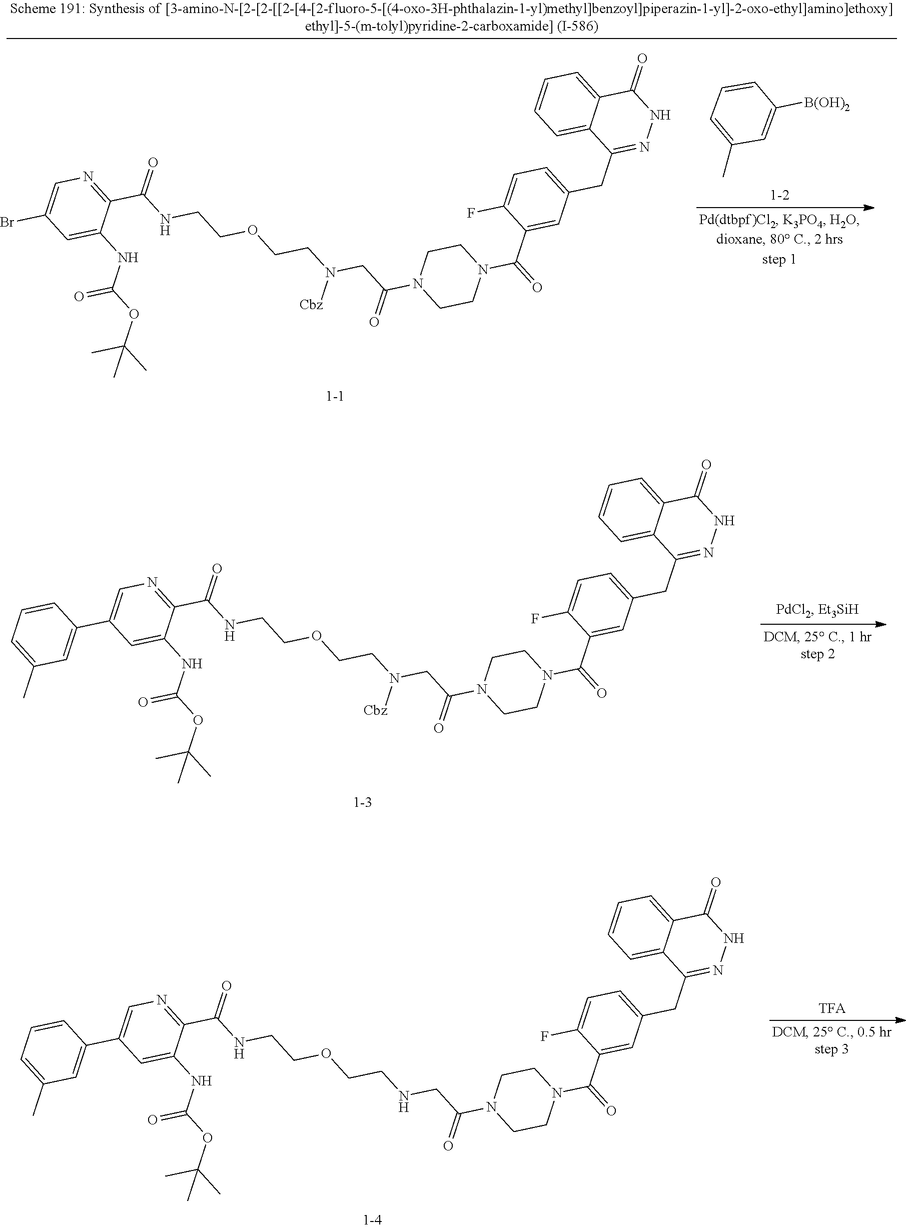

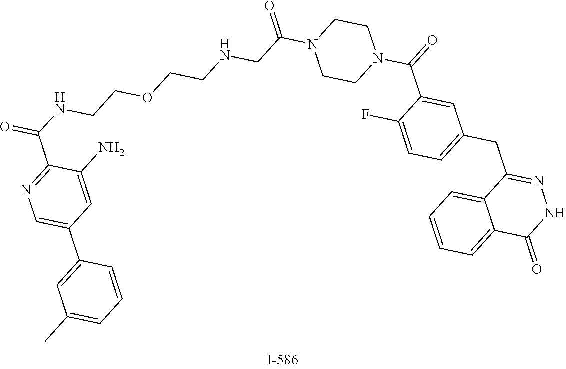 [SMILES: Cc1cccc(-c2cnc(C(=O)NCCOCCNCC(=O)N3CCN(C(=O)c4cc(Cc5n[nH]c(=O)c6ccccc56)ccc4F)CC3)c(N)c2)c1]

### Step 1: Synthesis of Intermediate 1-3.

A mixture of Intermediate 1-1 (200 mg, 211.91 μmol, 1 eq), Intermediate 1-2 (28.81 mg, 211.91 μmol, 1 eq), Pd(dtbpf)Cl₂ (13.81 mg, 21.19 μmol, 0.1 eq), and tripotassium;phosphate (134.94 mg, 635.72 mol, 3 eq) in dioxane (2.0 mL) and H₂O (0.4 mL) was degassed and purged with N₂ 3 times, and then the mixture was stirred at 80° C. for 2 hours under N₂ atmosphere. The reaction mixture was quenched by addition of H₂O (5 mL) at 25° C., and extracted with DCM (5 mL*3), the combined organic phase was dried over Na₂SO₄, filtered and concentrated under reduced pressure to give a crude product. The crude product was purified by reversed-phase HPLC (neutral condition), the eluent was concentrated and lyophilized to give a crude product. Intermediate 1-3 (170 mg, 177.73 μmol, 83.87% yield, 99.844% purity) was obtained as a yellow solid. LCMS (Method F): Retention time: 0.803 min, [M+H]⁺=955.3.

### Step 2: Synthesis of Intermediate 1-4.

To a solution of Intermediate 1-3 (60 mg, 62.82 μmol, 1 eq) in DCM (0.6 mL) was added PdCl₂ (5.57 mg, 31.41 μmol, 0.5 eq) and TEA (19.07 mg, 188.47 μmol, 26.23 μL, 3 eq). The reaction mixture was degassed with N₂. Then Et₃SiH (87.66 mg, 753.90 μmol, 120.41 μL, 12 eq) was added into the mixture. The mixture was stirred at 25° C. for 1 hour. The reaction mixture was quenched by addition of H₂O (3 mL) and extracted with DCM (3 mL*3), the combined organic phase was dried over Na₂SO₄, filtered and concentrated under reduced pressure to give a crude product. The crude product was used for the next step directly. Intermediate 1-4 (60 mg, crude) was obtained as a yellow solid. LCMS (Method D): Retention time: 0.435 min, [M+H]⁺=821.2.

### Step 3: Synthesis of I-586

To a solution of Intermediate 1-4 (30 mg, 36.55 μmol, 1 eq) in DCM (0.5 mL) was added TFA (153.50 mg, 1.35 mmol, 0.1 mL). The mixture was stirred at 25° C. for 0.5 hour. The mixture was dried with N₂ to give a crude product. The crude product was purified by prep-HPLC (column: CD01-Phenomenex luna C18 150*25*10 um; mobile phase: [water (TFA)-ACN]; gradient: 27%-57% B over 10 min), and the eluent was concentrated to remove ACN and lyophilized to give I-586 (10.98 mg, 15.23 μmol, 41.68% yield, 100% purity) as a white solid. LCMS (Method D): Retention time: 0.396 min, [M+H]⁺=721.3. ¹H NMR (400 MHz, METHANOL-d₄) δ=8.40-8.33 (m, 1H), 8.12-7.99 (m, 1H), 7.97-7.90 (m, 1H), 7.90-7.80 (m, 2H), 7.49 (d, J=5.6 Hz, 1H), 7.45-7.28 (m, 5H), 7.25-7.20 (m, 1H), 7.17-7.10 (m, 1H), 4.36 (d, J=5.6 Hz, 2H), 4.18-4.06 (m, 2H), 3.82 (t, J=4.2 Hz, 2H), 3.72 (s, 4H), 3.68-3.60 (m, 3H), 3.52-3.48 (m, 1H), 3.44 (t, J=4.8 Hz, 1H), 3.28 s, 5H), 2.40 (d, J=11.2 Hz, 3H). ¹⁹F NMR (376 MHz, METHANOL-d₄) δ=−77.224, −120.607.

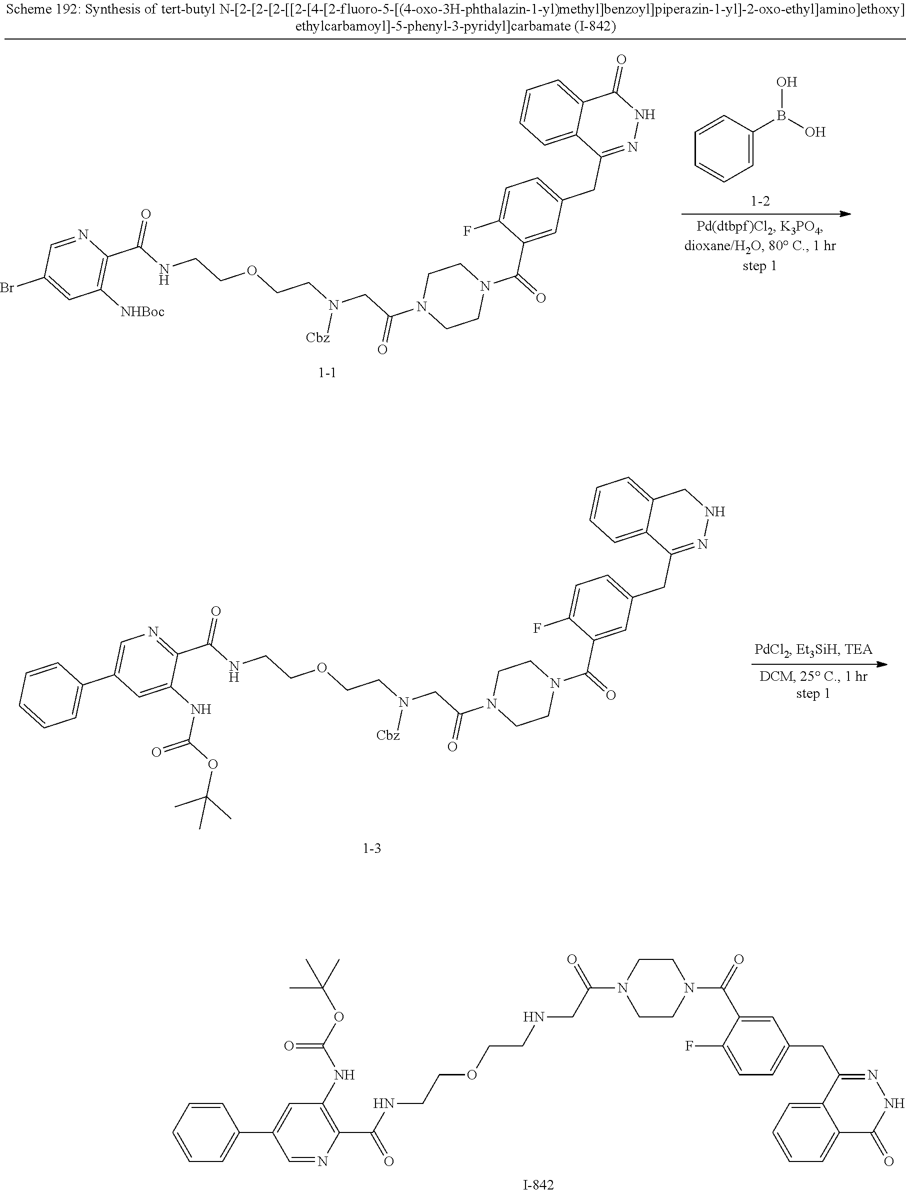 [SMILES: CC(C)(C)OC(=O)Nc1cc(Br)cnc1C(=O)NCCOCCN(CC(=O)N1CCN(C(=O)c2cc(Cc3n[nH]c(=O)c4ccccc34)ccc2F)CC1)C(=O)OCc1ccccc1 ; OB(O)c1ccccc1 ; CC(C)(C)OC(=O)Nc1cc(-c2ccccc2)cnc1C(=O)NCCOCCN(CC(=O)N1CCN(C(=O)c2cc(CC3=NNCc4ccccc43)ccc2F)CC1)C(=O)OCc1ccccc1 ; CC(C)(C)OC(=O)Nc1cc(-c2ccccc2)cnc1C(=O)NCCOCCNCC(=O)N1CCN(C(=O)c2cc(Cc3n[nH]c(=O)c4ccccc34)ccc2F)CC1]

### Step 1: Synthesis of Intermediate 1-3.

To a solution of Intermediate 1-1 (100 mg, 105.95 μmol, 1 eq) in H₂O (0.2 mL) and 1,4-dioxane (1 mL) was added Intermediate 1-2 (15.50 mg, 127.14 μmol, 1.2 eq), K₃PO₄ (67.47 mg, 317.86 μmol, 3 eq) and Pd(dtbpf)Cl₂ (6.91 mg, 10.60 μmol, 0.1 eq). The mixture was stirred at 80° C. for 1 hour under N₂. The reaction mixture was diluted with H₂O (5 mL) and extracted with EA (5 mL*2). The combined organic layers were dried over Na₂SO₄, filtered and concentrated under reduced pressure to give a residue. The residue was purified by flash silica gel chromatography (ISCO; 20 g SepaFlash Silica Flash Column, Eluent of 0˜100% Ethylacetate/Petroleum ether gradient @60 mL/min). The eluent was concentrated under reduced pressure to give Intermediate 1-3 (60 mg, 61.80 μmol, 58.33% yield, 96.923% purity) as a white solid. L CMS: Retention time (Method D): 0.550 min, [M+H]⁺=941.2. ¹H NMR (400 MHz, METHANOL-d4) δ=9.08-8.86 (m, 1H), 8.53-8.29 (m, 2H), 7.95-7.88 (m, 1H), 7.87-7.75 (m, 2H), 7.71-7.54 (m, 2H), 7.52-7.39 (m, 4H), 7.37-7.27 (m, 3H), 7.25-7.21 (m, 2H), 7.17-7.01 (m, 1H), 5.12 (s, 1H), 5.05-4.95 (m, 1H), 4.39-4.30 (m, 2H), 4.29-4.17 (m, 2H), 3.76-3.59 (m, 6H), 3.58-3.44 (m, 6H), 3.42-3.36 (m, 1H), 3.35-3.32 (m, 1H), 3.30-3.16 (m, 2H), 3.12-3.00 (m, 1H), 1.53 (s, 9H). ¹⁹F NMR (376 MHz, METHANOL-d4) δ=−77.502, −120.486.

### Step 2: Synthesis of I-842.

To a solution of Intermediate 1-3 (50 mg, 53.13 μmol, 1 eq) in DCM (1 mL) was added PdCl₂ (2.83 mg, 15.94 μmol, 0.3 eq) and TEA (10.75 mg, 106.27 μmol, 14.79 μL, 2 eq). The reaction mixture was degassed with N₂. Then a solution of Et₃SiH (24.71 mg, 212.54 μmol, 33.95 μL, 4 eq) in DCM (0.5 mL) was added dropwise at 10° C. The mixture was stirred at 25° C. for 1 hour. The reaction mixture was diluted with H₂O (2 mL) and extracted with DCM (5 mL*2). The combined organic layers were dried over Na₂SO₄, filtered and concentrated under reduced pressure to give a residue. The crude product was purified by reversed-phase HPLC (neutral condition). The eluent was concentrated under reduced pressure to remove ACN and then lyophilized to give I-842 (12.12 mg, 15.02 μmol, 28.27% yield, 100% purity) as a white solid. LCMS (Method D): Retention time: 0.429 min, (M+H)=807.2. ¹H NMR (400 MHz, METHANOL-d₄) δ=9.02 (s, 1H), 8.48 (d, J=6.4 Hz, 1H), 8.36 (d, J=7.6 Hz, 1H), 7.97-7.91 (m, 1H), 7.89-7.79 (m, 2H), 7.69 (d, J=7.2 Hz, 2H), 7.55-7.41 (m, 4H), 7.36-7.30 (m, 1H), 7.19-7.09 (m, 1H), 4.37 (d, J=6.0 Hz, 2H), 3.76-3.58 (m, 10H), 3.56 (s, 1H), 3.53-3.42 (m, 4H), 3.23-3.20 (m, 1H), 2.86-2.80 (m, 2H), 1.54 (s, 9H). ¹⁹F NMR (376 MHz, METHANOL-d₄) δ=−120.674.

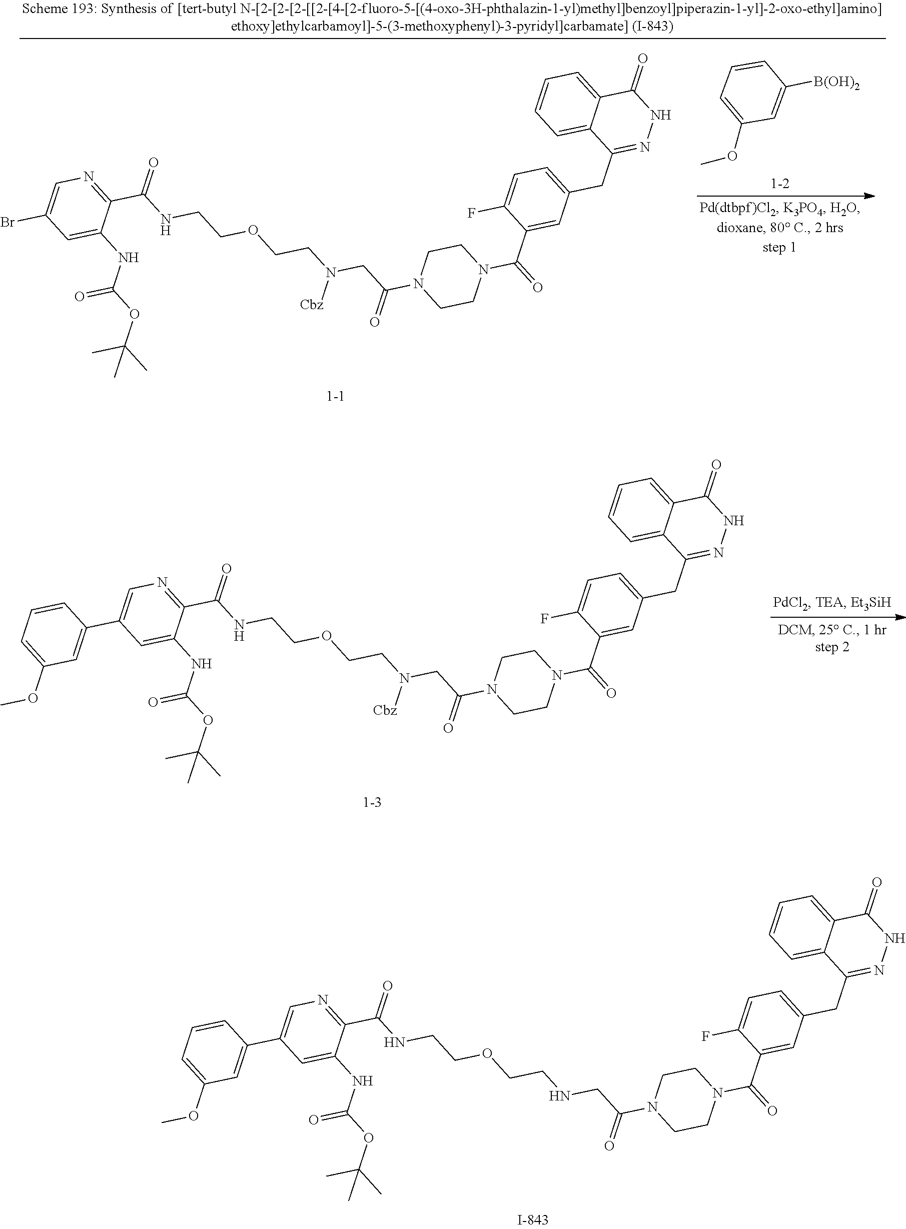 [SMILES: CC(C)(C)OC(=O)Nc1cc(Br)cnc1C(=O)NCCOCCN(CC(=O)N1CCN(C(=O)c2cc(Cc3n[nH]c(=O)c4ccccc34)ccc2F)CC1)C(=O)OCc1ccccc1 ; COc1cccc(B(O)O)c1 ; COc1cccc(-c2cnc(C(=O)NCCOCCN(CC(=O)N3CCN(C(=O)c4cc(Cc5n[nH]c(=O)c6ccccc56)ccc4F)CC3)C(=O)OCc3ccccc3)c(NC(=O)OC(C)(C)C)c2)c1 ; COc1cccc(-c2cnc(C(=O)NCCOCCNCC(=O)N3CCN(C(=O)c4cc(Cc5n[nH]c(=O)c6ccccc56)ccc4F)CC3)c(NC(=O)OC(C)(C)C)c2)c1]

### Step 1: Synthesis of Intermediate 1-3

A mixture of Intermediate 1-1 (250 mg, 264.88 μmol, 1 eq), Intermediate 1-2 (40.25 mg, 264.88 mol, 1 eq), ditert-butyl(cyclopenta-1,4-dien-1-yl)phosphane;dichloropalladium;iron (17.26 mg, 26.49 mol, 0.1 eq), and tripotassium;phosphate (168.68 mg, 794.65 μmol, 3 eq) in dioxane (2.0 mL) and H₂O (0.4 mL) was degassed and purged with N₂ 3 times, and then the mixture was stirred at 80° C. for 2 hr under N₂ atmosphere. The reaction mixture was quenched by addition of H₂O (2 mL) and extracted with DCM (1 mL*3), dried over Na₂SO₄, filtered and concentrated under reduced pressure to give a crude product. The crude product was purified by reversed-phase HPLC (neutral condition) and lyophilized to give Intermediate 1-3 (250 mg, 256.58 μmol, 96.86% yield, 99.659% purity) as a white solid. LCMS (Method F): Retention time: 0.550 min, [M+H]⁺=971.2.

### Step 2: Synthesis of I-843.

To a solution of Intermediate 1-3 (50 mg, 51.49 μmol, 1 eq) in DCM (0.6 mL) was added PdCl₂ (4.57 mg, 25.75 μmol, 0.5 eq) and TEA (15.63 mg, 154.47 μmol, 21.50 μL, 3 eq). The reaction mixture was degassed with N₂. Then Et₃SiH (71.85 mg, 617.90 μmol, 98.69 μL, 12 eq) was added. The mixture was stirred at 25° C. for 1 hr. The reaction mixture was quenched by addition of H₂O (0.5 mL) at 25° C., and extracted with DCM (1 mL*3), dried over Na₂SO₄, filtered and concentrated under reduced pressure to give a crude product. The crude product was purified by reversed-phase HPLC (0.1% NH₃·H₂O condition) and prep-HPLC (column: CD01-Phenomenex luna C18 150*25*10 um; mobile phase: [water(FA)-ACN]; gradient: 22%-52% B over 11 min), the eluent was concentrated under reduced pressure to remove ACN and then lyophilized to give I-843 (13.64 mg, 15.45 μmol, 30.00% yield, 100% purity, FA salt) as a white solid. LCMS (Method D): Retention time: 0.434 min, [M+H]⁺=837.3. ¹H NMR (400 MHz, METHANOL-d₄) δ=8.98 (d, J=2.8 Hz, 1H), 8.54 (s, 1H), 8.46 (d, J=9.2 Hz, 1H), 8.39-8.32 (m, 1H), 7.96-7.90 (m, 1H), 7.89-7.78 (m, 2H), 7.50-7.43 (m, 1H), 7.43-7.35 (m, 1H), 7.35-7.28 (m, 1H), 7.27-7.08 (m, 3H), 7.05-6.95 (m, 1H), 4.36 (d, J=5.6 Hz, 2H), 3.85 (d, J=15.2 Hz, 3H), 3.75-3.59 (m, 11H), 3.51-3.44 (m, 2H), 3.29-3.18 (m, 3H), 3.00-2.90 (m, 2H), 1.54 (s, 9H). ¹⁹F NMR (376 MHz, METHANOL-d₄) δ=−120.642.

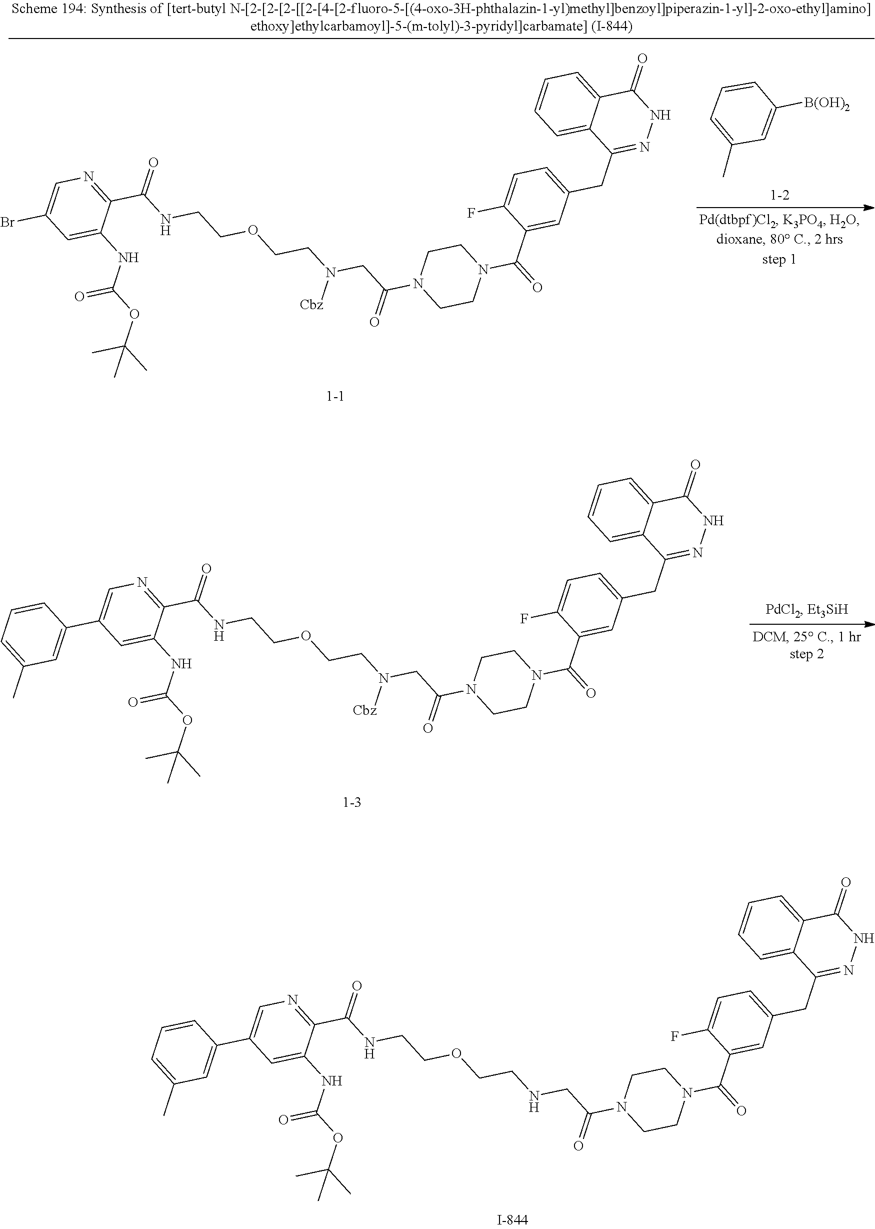

### Step 1: Synthesis of Intermediate 1-3.

A mixture of Intermediate 1-1 (200 mg, 211.91 μmol, 1 eq), Intermediate 1-2 (28.81 mg, 211.91 mol, 1 eq), ditert-butyl(cyclopenta-1,4-dien-1-yl)phosphane;dichloropalladium;iron (13.81 mg, 21.19 μmol, 0.1 eq), and tripotassium;phosphate (134.94 mg, 635.72 μmol, 3 eq) in dioxane (2.0 mL) and H₂O (0.4 mL) was degassed and purged with N₂ 3 times, and then the mixture was stirred at 80° C. for 2 hr under N₂ atmosphere. The reaction mixture was quenched by addition of H₂O (2 mL) at 25° C., and extracted with DCM (1 mL*3), dried over Na₂SO₄, filtered and concentrated under reduced pressure to give a residue. The crude product was purified by reversed-phase HPLC (neutral condition) and lyophilized to give Intermediate 1-3 (170 mg, 177.73 μmol, 83.87% yield, 99.844% purity) as a yellow solid. LCMS (Method F): Retention time: 0.803 min, [M+H]⁺=955.3.

### Step 2: Synthesis of I-844.

To a solution of Intermediate 1-3 (60 mg, 62.82 μmol, 1 eq) in DCM (0.6 mL) was added PdCl₂ (5.57 mg, 31.41 μmol, 0.5 eq) and TEA (19.07 mg, 188.47 μmol, 26.23 μL, 3 eq). The reaction mixture was degassed with N₂. Then Et₃SiH (87.66 mg, 753.90 μmol, 120.41 μL, 12 eq) was added. The mixture was stirred at 25° C. for 1 hr. The reaction mixture was quenched by addition of H₂O (2 mL) and extracted with DCM (2 mL*3), dried over Na₂SO₄, filtered and concentrated under reduced pressure to give a crude product. The crude product was purified by prep-HPLC (column: CD01-Phenomenex luna C18 150*25*10 um; mobile phase: [water(FA)-ACN]; gradient: 25%-55% B over 8 min) and lyophilized to give I-844 (12.74 mg, 15.52 μmol, 42.47% yield, 100% purity) as a white solid. LCMS (Method D): Retention time: 0.417 min, [M+H]⁺=821.3. ¹H NMR (400 MHz, METHANOL-d₄) δ=8.99 (s, 1H), 8.54 (s, 1H), 8.46 (d, J=8.8 Hz, 1H), 8.38-8.33 (m, 1H), 7.95-7.90 (m, 1H), 7.88-7.79 (m, 2H), 7.52-7.44 (m, 3H), 7.42-7.34 (m, 1H), 7.33-7.32 (m, 1H), 7.29-7.23 (m, 1H), 7.17-7.09 (m, 1H), 4.36 (d, J=6.4 Hz, 2H), 3.76-3.59 (m, 11H), 3.51-3.44 (m, 2H), 3.30-3.19 (m, 3H), 2.99-2.89 (m, 2H), 2.42 (d, J=14.8 Hz, 3H), 1.54 (s, 9H). ¹⁹F NMR (376 MHz, METHANOL-d₄) δ=−120.644.

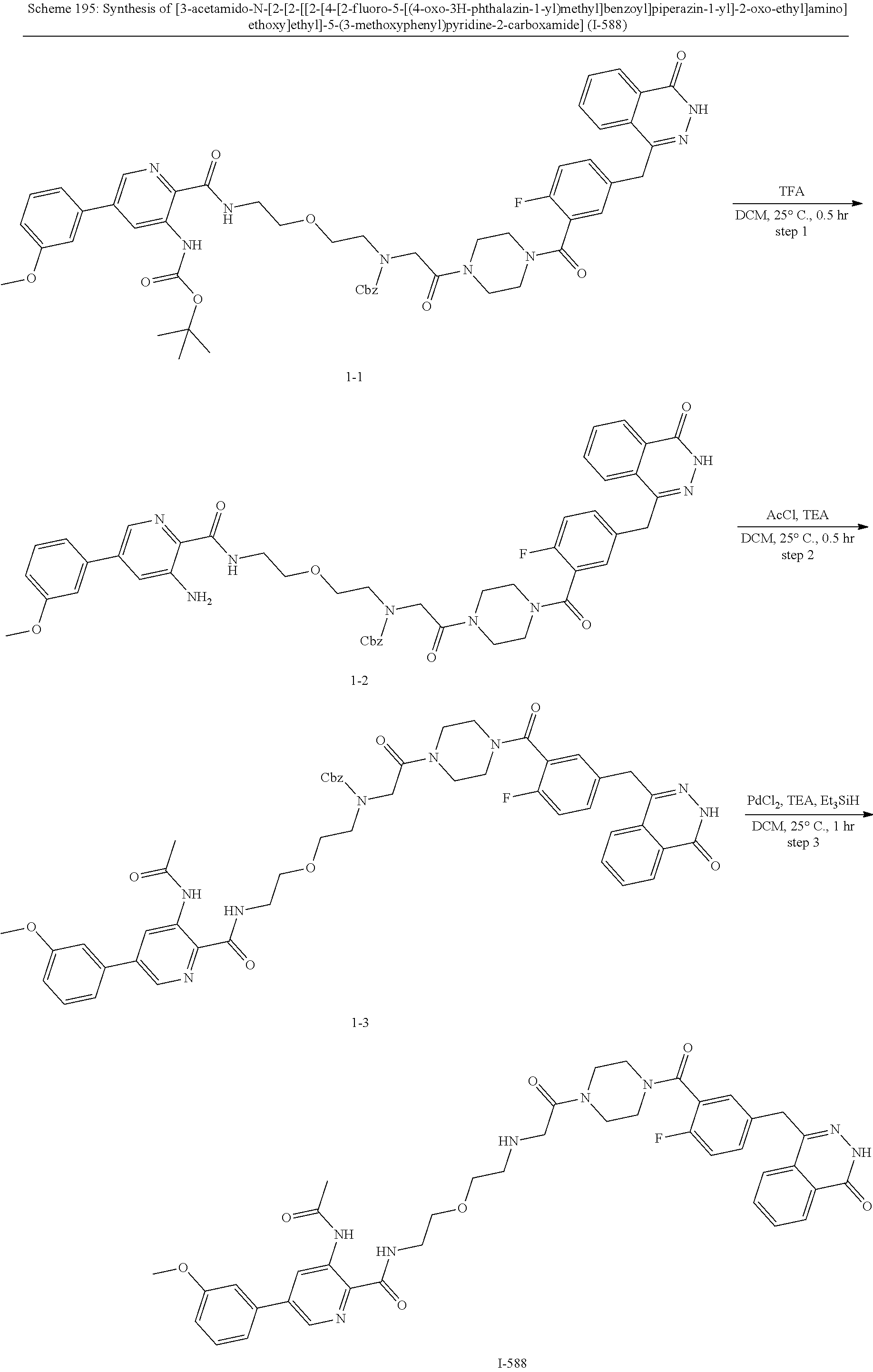 [SMILES: COc1cccc(-c2cnc(C(=O)NCCOCCN(CC(=O)N3CCN(C(=O)c4cc(Cc5n[nH]c(=O)c6ccccc56)ccc4F)CC3)C(=O)OCc3ccccc3)c(NC(=O)OC(C)(C)C)c2)c1 ; COc1cccc(-c2cnc(C(=O)NCCOCCN(CC(=O)N3CCN(C(=O)c4cc(Cc5n[nH]c(=O)c6ccccc56)ccc4F)CC3)C(=O)OCc3ccccc3)c(N)c2)c1 ; COc1cccc(-c2cnc(C(=O)NCCOCCN(CC(=O)N3CCN(C(=O)c4cc(Cc5n[nH]c(=O)c6ccccc56)ccc4F)CC3)C(=O)OCc3ccccc3)c(NC(C)=O)c2)c1 ; COc1cccc(-c2cnc(C(=O)NCCOCCNCC(=O)N3CCN(C(=O)c4cc(Cc5n[nH]c(=O)c6ccccc56)ccc4F)CC3)c(NC(C)=O)c2)c1]

### Step 1: Synthesis of Intermediate 1-2

To a solution of Intermediate 1-1 (100 mg, 102.98 μmol, 1 eq) in DCM (1 mL) was added TFA (307.00 mg, 2.69 mmol, 0.2 mL). The mixture was stirred at 25° C. for 0.5 hr. The mixture was dried by a stream of N₂ to give a crude product. Without further purification, the crude product was used in the next step. Intermediate 1-2 (100 mg, crude, TFA salt) was obtained as a yellow solid. LCMS (Method D): Retention time: 0.468 min, [M+H]⁺=871.2.

### Step 2: Synthesis of Intermediate 1-3

To a solution of Intermediate 1-2 (100 mg, 101.53 μmol, 1 eq, TFA salt) in DCM (1 mL) was added TEA (20.55 mg, 203.06 μmol, 28.26 μL, 2 eq) and acetyl chloride (7.97 mg, 101.53 μmol, 7.22 μL, 1 eq). The mixture was stirred at 25° C. for 0.5 hr. The reaction mixture was quenched by addition of H₂O (1 mL) and extracted with DCM (1 mL*3), the combined organic phase was dried over Na₂SO₄, filtered and concentrated under reduced pressure to give a crude product. The crude product was purified by reversed-phase HPLC (0.1% FA condition) and lyophilized to give a crude product. Intermediate 1-3 (80 mg, 87.63 μmol, 86.31% yield, 100% purity) was obtained as a white solid. LCMS (Method D): Retention time: 0.480 min, [M+H]⁺=913.2.

### Step 3: Synthesis of I-588

To a solution of Intermediate 1-3 (70 mg, 76.67 μmol, 1 eq) in DCM (0.7 mL) was added PdCl₂ (6.80 mg, 38.34 μmol, 0.5 eq) and TEA (23.28 mg, 230.02 μmol, 32.02 μL, 3 eq). The reaction mixture was degassed and purged with N₂ 3 times. Then Et₃SiH (106.98 mg, 920.09 μmol, 146.96 μL, 12 eq) was added. The mixture was stirred at 25° C. for 1 hr. The reaction mixture was quenched by addition of H₂O (3 mL), and extracted with DCM (4 mL*3), the combined organic phase was dried over Na₂SO₄, filtered and concentrated under reduced pressure to give a crude product. The crude product was purified by prep-HPLC (column: CD01-Phenomenex luna C18 150*25*10 um; mobile phase: [water (FA)-ACN]; gradient: 15%-45% B over 8 min) and lyophilized to give I-588 (21.04 mg, 25.51 μmol, 33.27% yield, 100% purity, FA salt) as a white solid. LCMS (Method E): Retention time: 0.369 min, [M+H]⁺=779.4. ¹H NMR (400 MHz, METHANOL-d₄) δ=9.23 (s, 1H), 8.57-8.49 (m, 1H), 8.40-8.31 (m, 1H), 7.97-7.89 (m, 1H), 7.89-7.79 (m, 2H), 7.47 (d, J=3.2 Hz, 1H), 7.44 (s, 1H), 7.34-7.28 (m, 1H), 7.25-7.09 (m, 3H), 7.03-6.98 (m, 1H), 4.36 (d, J=3.6 Hz, 2H), 3.93-3.53 (m, 15H), 3.46 (d, J=3.2 Hz, 2H), 3.22 (d, J=2.4 Hz, 3H), 3.07-2.99 (m, 2H), 2.24 (s, 3H), ¹⁹F NMR (376 MHz, METHANOL-d₄) δ=−120.674.

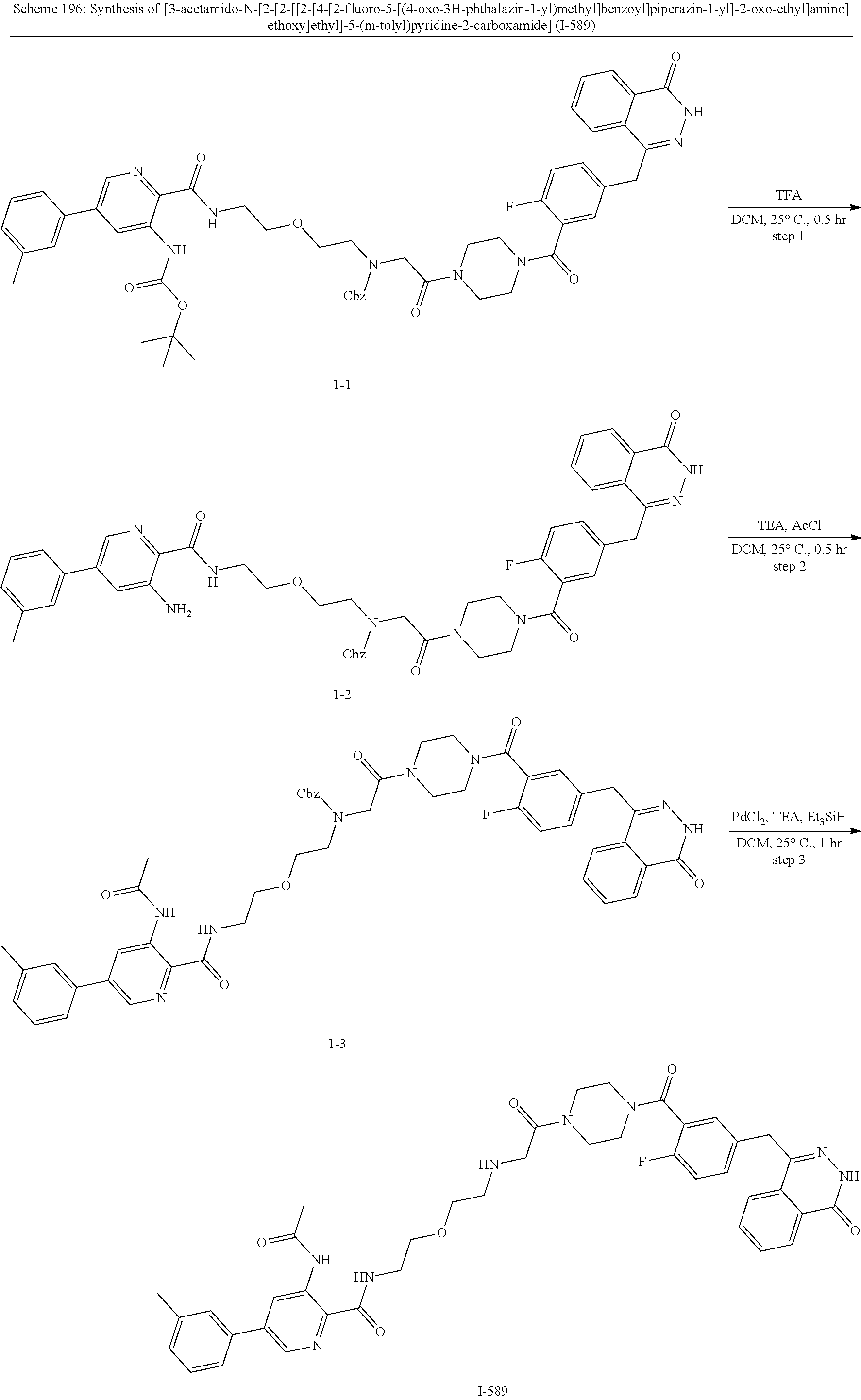 [SMILES: Cc1cccc(-c2cnc(C(=O)NCCOCCN(CC(=O)N3CCN(C(=O)c4cc(Cc5n[nH]c(=O)c6ccccc56)ccc4F)CC3)C(=O)OCc3ccccc3)c(NC(=O)OC(C)(C)C)c2)c1 ; Cc1cccc(-c2cnc(C(=O)NCCOCCN(CC(=O)N3CCN(C(=O)c4cc(Cc5n[nH]c(=O)c6ccccc56)ccc4F)CC3)C(=O)OCc3ccccc3)c(N)c2)c1 ; CC(=O)Nc1cc(-c2cccc(C)c2)cnc1C(=O)NCCOCCN(CC(=O)N1CCN(C(=O)c2cc(Cc3n[nH]c(=O)c4ccccc34)ccc2F)CC1)C(=O)OCc1ccccc1 ; CC(=O)Nc1cc(-c2cccc(C)c2)cnc1C(=O)NCCOCCNCC(=O)N1CCN(C(=O)c2cc(Cc3n[nH]c(=O)c4ccccc34)ccc2F)CC1]

### Step 1: Synthesis of Intermediate 1-2.

To a solution of Intermediate 1-1 (90 mg, 94.24 μmol, 1 eq) in DCM (1 mL) was added TFA (307.00 mg, 2.69 mmol, 0.2 mL). The mixture was stirred at 25° C. for 0.5 hr. The mixture was dried by a stream of N₂ to give a crude product. Without further purification, the crude product was used in the next step. Intermediate 1-2 (90 mg, crude, TFA salt) was obtained as a yellow solid. LCMS (Method D): Retention time: 0.484 min, [M+H]⁺=855.6.

### Step 2: Synthesis of Intermediate 1-3.

To a solution of Intermediate 1-2 (90 mg, 92.88 μmol, 1 eq, TFA salt) in DCM (1 mL) was added TEA (18.80 mg, 185.77 μmol, 25.86 μL, 2 eq) and acetyl chloride (7.29 mg, 92.88 μmol, 6.60 μL, 1 eq). The mixture was stirred at 25° C. for 0.5 hr. The reaction mixture was quenched by addition of H₂O (1 mL) and extracted with DCM (1 mL*3), dried over Na₂SO₄, filtered and concentrated under reduced pressure to give a crude product. The crude product was purified by reversed-phase HPLC (0.1% FA condition) and lyophilized to give Intermediate 1-3 (55 mg, 60.99 μmol, 65.66% yield, 99.460% purity) as a white solid. LCMS (Method D): Retention time: 0.498 min, [M+H]⁺=897.2.

### Step 3: Synthesis of I-589

To a solution of Intermediate 1-3 (45 mg, 50.17 μmol, 1 eq) in DCM (0.5 mL) was added PdCl₂ (4.45 mg, 25.08 μmol, 0.5 eq) and TEA (15.23 mg, 150.51 μmol, 20.95 μL, 3 eq). The reaction mixture was degassed with N₂. Then Et₃SiH (70.00 mg, 602.03 μmol, 96.16 μL, 12 eq) was added. The mixture was stirred at 25° C. for 1 hr. The reaction mixture was quenched by addition of H₂O (1 mL) and extracted with DCM (2 mL*3), dried over Na₂SO₄, filtered and concentrated under reduced pressure to give a residue. The crude product was purified by reversed-phase HPLC (0.1% FA condition) and lyophilized to give I-589 (16.5 mg, 20.24 μmol, 40.34% yield, 99.209% purity, FA salt) as a white solid. LCMS (Method D): Retention time: 0.371 min, [M+H]⁺=763.3. ¹H NMR (400 MHz, METHANOL-d₄) δ=9.21 (s, 1H), 8.55-8.48 (m, 2H), 8.35 (d, J=7.6 Hz, 1H), 7.95-7.89 (m, 1H), 7.87-7.79 (m, 2H), 7.48-7.22 (m, 6H), 7.16-7.09 (m, 1H), 4.35 (d, J=3.2 Hz, 2H), 3.80-3.59 (m, 12H), 3.46 (d, J=4.4 Hz, 2H), 3.29-3.20 (m, 2H), 2.99 (d, J=4.0 Hz, 2H), 2.40 (d, J=14.8 Hz, 3H), 2.23 (s, 3H). ¹⁹F NMR (376 MHz, METHANOL-d₄) δ=−120.635.

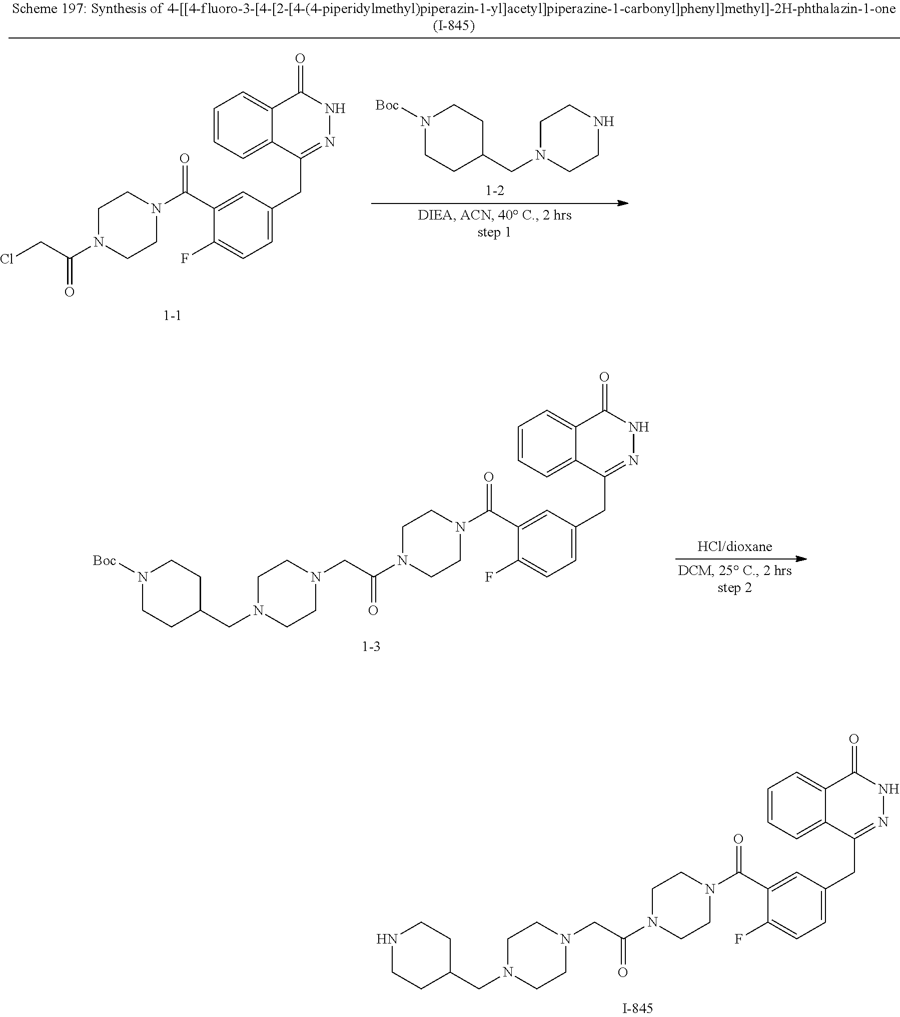 [SMILES: O=C(CCl)N1CCN(C(=O)c2cc(Cc3n[nH]c(=O)c4ccccc34)ccc2F)CC1 ; CC(C)(C)OC(=O)N1CCC(CN2CCNCC2)CC1 ; CC(C)(C)OC(=O)N1CCC(CN2CCN(CC(=O)N3CCN(C(=O)c4cc(Cc5n[nH]c(=O)c6ccccc56)ccc4F)CC3)CC2)CC1 ; O=C(CN1CCN(CC2CCNCC2)CC1)N1CCN(C(=O)c2cc(Cc3n[nH]c(=O)c4ccccc34)ccc2F)CC1]

### Step 1: Synthesis of Intermediate 1-3

To a solution of Intermediate 1-1 (10 g, 22.58 mmol, 1 eq) in ACN (100 mL) was added DIEA (11.67 g, 90.32 mmol, 15.73 mL, 4 eq) and Intermediate 1-2 (7.68 g, 27.10 mmol, 1.2 eq). The mixture was stirred at 40° C. for 2 hr. The residue was diluted with H₂O (50 mL) and extracted with EA (50 mL*3), dried over Na₂SO₄, filtered and concentrated under reduced pressure to give a residue. The residue was purified by flash silica gel chromatography (ISCO; 330 g SepaFlash Silica Flash Column, Eluent of 0˜-100% Dichloromethane/Methanol gradient@100 mL/min). The eluent was concentrated under reduced pressure to give a residue. To the residue was added H₂O and ACN and lyophilized to give Intermediate 1-3 (12 g, 13.92 mmol, 61.65% yield, 80.022% purity) as a yellow solid. LCMS (Method D): Rt=0.324 min, [M+H]⁺=690.3. ¹H NMR (400 MHz, METHANOL-d₄) δ=8.37 (d, J=7.6 Hz, 1H), 7.99-7.92 (m, 1H), 7.90-7.82 (m, 2H), 7.49 (d, J=2.4 Hz, 1H), 7.38 (t, J=6.0 Hz, 1H), 7.16 (t, J=8.8 Hz, 1H), 4.39 (s, 2H), 4.05 (d, J=12.4 Hz, 3H), 3.81-3.63 (m, 5H), 3.53 (d, J=18.4 Hz, 2H), 2.77 (s, 2H), 2.49 (d, J=18.8 Hz, 8H), 2.22-2.17 (m, 2H), 1.74 (d, J=12.4 Hz, 5H), 1.45 (s, 9H), 1.11-0.97 (m, 3H).

### Step 2: Synthesis of I-845

To Intermediate 1-3 (6 g, 8.70 mmol, 1 eq) was added HCl/dioxane (2 M, 60 mL, 13.80 eq). The mixture was stirred at 25° C. for 2 hr. The reaction liquid was concentrated by reducing pressure to obtain the crude product. Then 50 mg of crude product was purified by reversed-phase HPLC (0.1% HCl condition), which was concentrated and lyophilized to give I-845 (19.94 mg, 31.84 μmol, 39.88% yield, 100.00% purity, HCl salt) as a white solid. LCMS (Method D): Rt=0.220 min, [M+H]⁺=590.2. ¹H NMR (400 MHz, METHANOL-d₄) δ=8.37 (d, J=7.6 Hz, 1H), 8.00-7.93 (m, 1H), 7.92-7.79 (m, 2H), 7.51 (d, J=1.6 Hz, 1H), 7.41-7.35 (m, 1H), 7.18 (t, J=9.2 Hz, 1H), 4.58 (d, J=17.6 Hz, 2H), 4.40 (s, 2H), 3.93 (br s, 4H), 3.87-3.63 (m, 7H), 3.56 (d, J=3.6 Hz, 2H), 3.49-3.39 (m, 4H), 3.34 (d, J=1.6 Hz, 3H), 3.13-3.01 (m, 2H), 2.33 (d, J=1.6 Hz, 1H), 2.22 (d, J=13.2 Hz, 2H), 1.65-1.49 (m, 2H). ¹⁹F NMR (376 MHz, METHANOL-d₄) δ=−120.725.

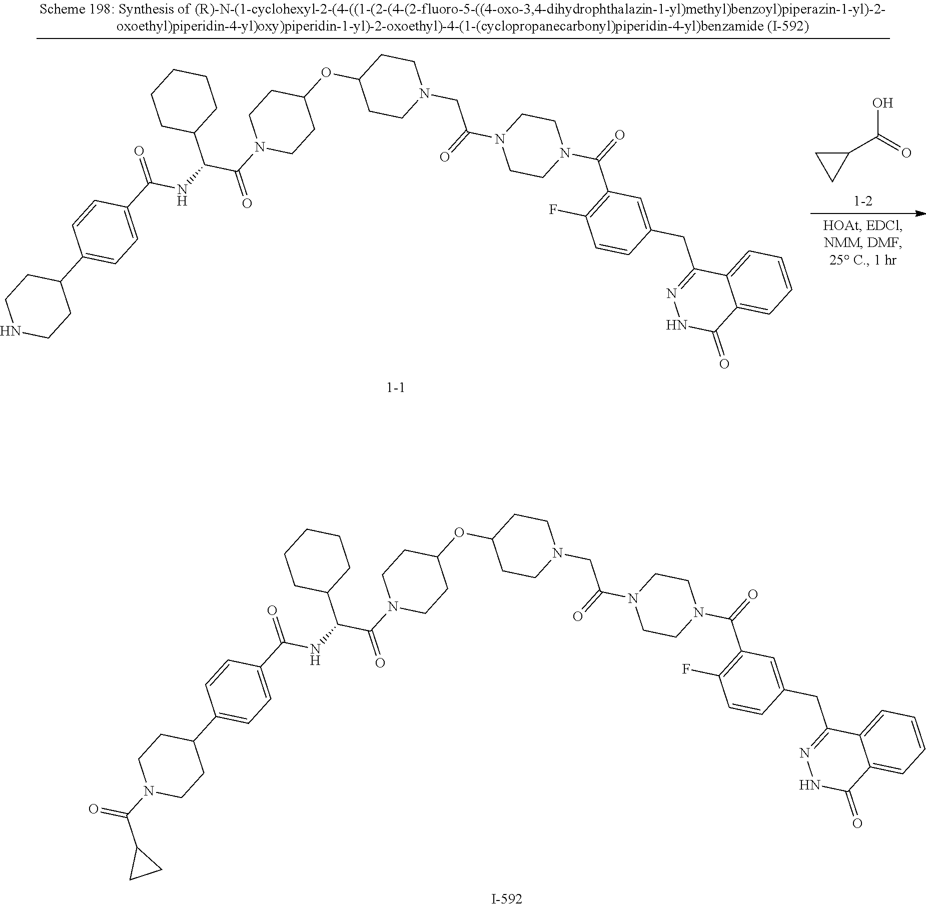 [SMILES: O=C(N[C@@H](C(=O)N1CCC(OC2CCN(CC(=O)N3CCN(C(=O)c4cc(Cc5n[nH]c(=O)c6ccccc56)ccc4F)CC3)CC2)CC1)C1CCCCC1)c1ccc(C2CCNCC2)cc1 ; O=C(O)C1CC1 ; O=C(N[C@@H](C(=O)N1CCC(OC2CCN(CC(=O)N3CCN(C(=O)c4cc(Cc5n[nH]c(=O)c6ccccc56)ccc4F)CC3)CC2)CC1)C1CCCCC1)c1ccc(C2CCN(C(=O)C3CC3)CC2)cc1]

To a solution of Intermediate 1-1 (100 mg, 104.87 μmol, 1 eq, HCl salt) and Intermediate 1-2 (9.03 mg, 104.87 μmol, 8.30 μL, 1 eq) in DMF (1 mL) was added EDCI (40.21 mg, 209.74 μmol, 2 eq), HOAt (14.27 mg, 104.87 μmol, 14.67 μL, 1 eq) and NMM (53.04 mg, 524.34 μmol, 57.65 μL, 5 eq). The mixture was stirred at 25° C. for 1 hr. To the reaction mixture was added H₂O (2 mL) and then extracted with DCM (2 mL*3), the combined organic phase was washed, with dried by Na₂SO₄, filtered and concentrated. The residue was purified by prep-HPLC (column: CD01-Phenomenex luna C18 150*25*10 um; mobile phase: [water (FA)-ACN]; gradient: 20%-50% B over 10 min), the eluent was concentrated to remove ACN and lyophilized to give I-592 (21.94 mg, 21.19 μmol, 20.21% yield, 99.599% purity, FA salt) as a white solid LCMS (Method D): Retention time: 0.385 min, [M+H]⁺=985.4. SFC: Retention time: 1.424 min. ¹H NMR (400 MHz, METHANOL-d₄) δ=8.53 (d, J=2.0 Hz, 1H), 8.39 (d, J=7.6 Hz, 1H), 8.02-7.94 (m, 1H), 7.94-7.83 (m, 2H), 7.80 (d, J=8.0 Hz, 2H), 7.51 (d, J=3.6 Hz, 1H), 7.39 (d, J=8.0 Hz, 3H), 7.19 (t, J=9.2 Hz, 1H), 4.94 (d, J=8.4 Hz, 1H), 4.73-4.65 (m, 1H), 4.55-4.46 (m, 1H), 4.41 (s, 2H), 4.11-4.00 (m, 1H), 4.00-3.85 (m, 1H), 3.84-3.74 (m, 3H), 3.70 (s, 2H), 3.66-3.51 (m, 4H), 3.51-3.40 (m, 2H), 3.39-3.34 (m, 2H), 3.29-3.24 (m, 1H), 3.01-2.75 (m, 4H), 2.58-2.34 (m, 2H), 2.11-1.99 (m, 1H), 1.91 (d, J=12.0 Hz, 8H), 1.81-1.75 (m, 2H), 1.71 (d, J=11.6 Hz, 4H), 1.66-1.55 (m, 3H), 1.55-1.43 (m, 1H), 1.41-1.18 (m, 4H), 1.18-1.05 (m, 2H), 0.97-0.88 (m, 2H), 0.84 (d, J=8.0 Hz, 2H). ¹⁹F NMR (376 MHz, METHANOL-d₄) δ=−120.787.

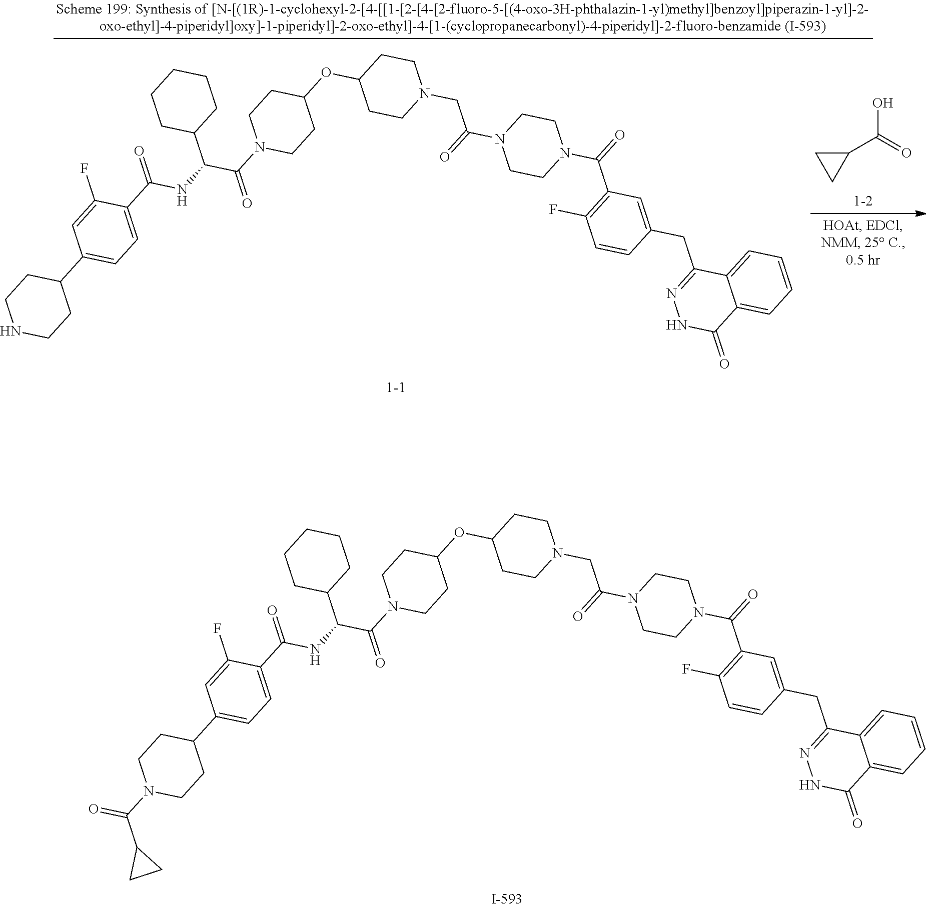 [SMILES: O=C(N[C@@H](C(=O)N1CCC(OC2CCN(CC(=O)N3CCN(C(=O)c4cc(Cc5n[nH]c(=O)c6ccccc56)ccc4F)CC3)CC2)CC1)C1CCCCC1)c1ccc(C2CCNCC2)cc1F ; O=C(O)C1CC1 ; O=C(N[C@@H](C(=O)N1CCC(OC2CCN(CC(=O)N3CCN(C(=O)c4cc(Cc5n[nH]c(=O)c6ccccc56)ccc4F)CC3)CC2)CC1)C1CCCCC1)c1ccc(C2CCN(C(=O)C3CC3)CC2)cc1F]

To a solution of Intermediate 1-1 (70 mg, 72.05 μmol, 1 eq, HCl salt) in DMF (0.7 mL) was added EDCI (41.44 mg, 216.15 μmol, 3 eq), NMM (36.44 mg, 360.24 μmol, 39.61 μL, 5 eq), Intermediate 1-2 (6.20 mg, 72.05 μmol, 5.70 μL, 1 eq) and HOAt (9.81 mg, 72.05 μmol, 10.08 μL, 1 eq). The mixture was stirred at 25° C. for 0.5 h. The reaction mixture was quenched by addition H₂O (5 mL) and extracted with EA (5 mL*3), the combined organic layers were dried over Na₂SO₄, filtered and concentrated under reduced pressure to give a residue. The crude product was purified by prep-HPLC (column: CD01-Phenomenex luna C18 150*25*10 um; mobile phase: [water(FA)-ACN]; gradient: 18%-48% B over 8 min) and the eluent was lyophilized to give I-593 (29.42 mg, 29.27 μmol, 40.62% yield, 99.791% purity) as a white solid. LCMS (Method D): Retention time: 0.438 min, [M+H]⁺=1003.5. ¹H NMR (400 MHz, METHANOL-d₄) δ=8.36 (d, J=7.6 Hz, 1H), 7.99-7.91 (m, 1H), 7.90-7.79 (m, 2H), 7.71 (t, J=8.0 Hz, 1H), 7.49 (s, 1H), 7.38 (s, 1H), 7.26-7.08 (m, 3H), 4.98 (d, J=6.8 Hz, 1H), 4.66 (d, J=13.2 Hz, 1H), 4.48 (d, J=13.6 Hz, 1H), 4.38 (s, 2H), 4.01 (t, J=11.6 Hz, 1H), 3.94-3.83 (m, 1H), 3.77 (d, J=17.6 Hz, 3H), 3.68 (s, 2H), 3.64-3.39 (m, 6H), 3.34 (s, 2H), 3.29-3.20 (m, 2H), 2.98-2.70 (m, 4H), 2.57-2.27 (m, 2H), 2.11-1.87 (m, 6H), 1.87-1.75 (m, 5H), 1.75-1.40 (m, 7H), 1.33-1.07 (m, 5H), 0.95-0.76 (m, 4H). ¹⁹F NMR (376 MHz, METHANOL-d₄) δ=−115.337, −120.740.

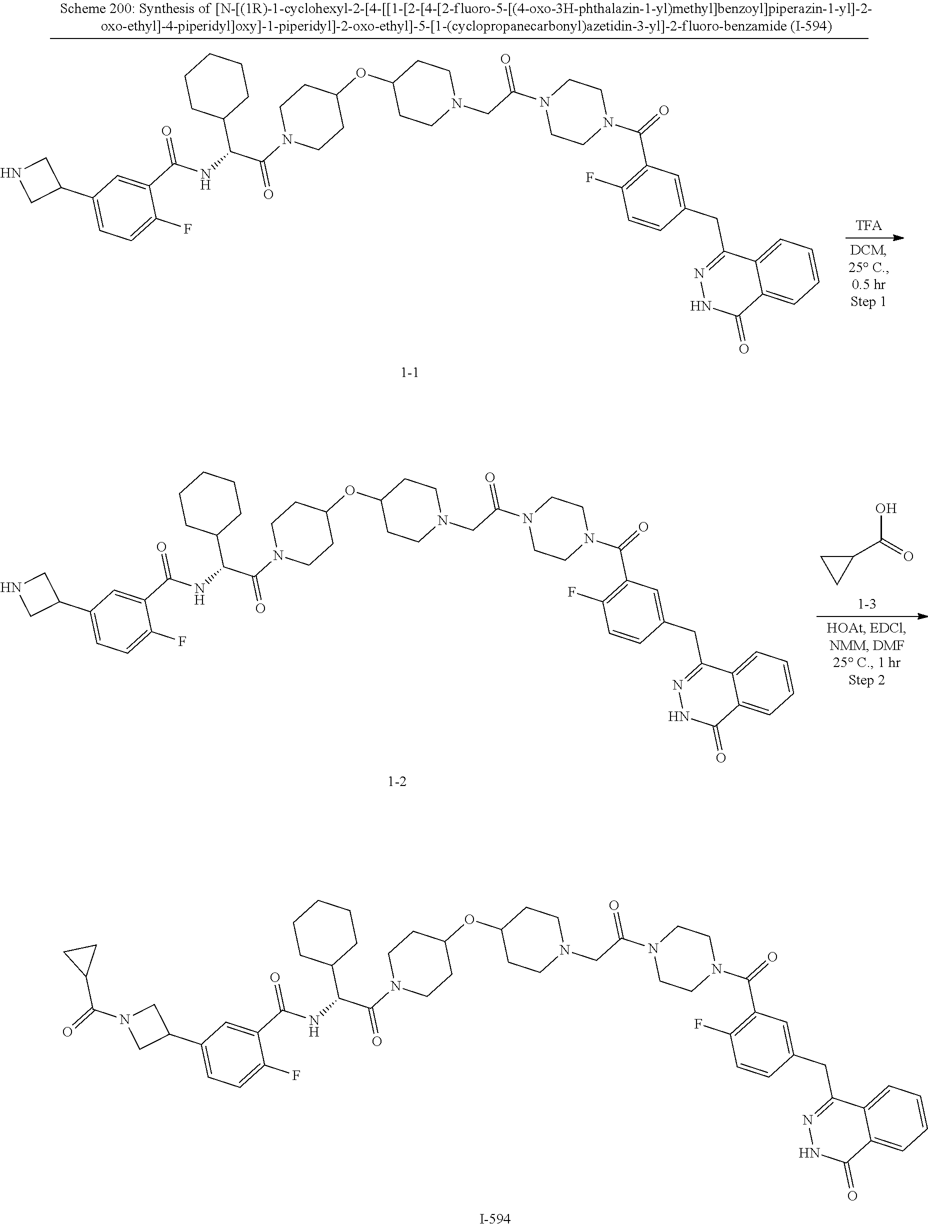 [SMILES: O=C(N[C@@H](C(=O)N1CCC(OC2CCN(CC(=O)N3CCN(C(=O)c4cc(Cc5n[nH]c(=O)c6ccccc56)ccc4F)CC3)CC2)CC1)C1CCCCC1)c1cc(C2CNC2)ccc1F ; O=C(N[C@@H](C(=O)N1CCC(OC2CCN(CC(=O)N3CCN(C(=O)c4cc(Cc5n[nH]c(=O)c6ccccc56)ccc4F)CC3)CC2)CC1)C1CCCCC1)c1cc(C2CNC2)ccc1F ; O=C(O)C1CC1 ; O=C(N[C@@H](C(=O)N1CCC(OC2CCN(CC(=O)N3CCN(C(=O)c4cc(Cc5n[nH]c(=O)c6ccccc56)ccc4F)CC3)CC2)CC1)C1CCCCC1)c1cc(C2CN(C(=O)C3CC3)C2)ccc1F]

### Step 1: Synthesis of Intermediate 1-2.

To a mixture of intermediate 1-1 (40 mg, 39.72 μmol, 1 eq) in DCM (0.5 mL) was added TFA (153.50 mg, 1.35 mmol, 0.1 mL, 33.90 eq) at 25° C., and the mixture was stirred at 25° C. for 0.5 h. The reaction mixture was concentrated to give a residue. The crude product was used in the next step without further purification. Intermediate 1-2 (40 mg, crude, TFA salt) was obtained as a yellow gum. LCMS (Method D): Retention time: 0.318 min, [M+H]⁺=907.4.

### Step 2: Synthesis of I-594

To a mixture of intermediate 1-2 (40 mg, 39.17 μmol, 1 eq, TFA salt) in DMF (0.5 mL) was added intermediate 1-3 (5.06 mg, 58.76 μmol, 4.65 μL, 1.5 eq), EDCI (22.53 mg, 117.52 μmol, 3 eq), HOAt (5.33 mg, 39.17 μmol, 5.48 μL, 1 eq) and NMM (19.81 mg, 195.87 μmol, 21.53 μL, 5 eq) at 25° C., and the mixture was stirred at 25° C. for 1 h. The reaction mixture was diluted with H₂O (2 mL) and extracted with DCM (2 mL×3), the combined organic layers were dried over Na₂SO₄, filtered and concentrated to give a residue. The residue was purified by reversed-phase HPLC (0.1% FA condition) and lyophilized to give I-594 (28.64 mg, 27.07 μmol, 69.10% yield, 96.517% purity, FA salt) as a white solid. LCMS (Method D): Retention time: 0.379 min, [M+H]⁺=975.4. SFC: Retention time: 0.837 min¹H NMR (400 MHz, METHANOL-d₄) δ=8.50 (s, 1H), 8.37 (d, J=7.6 Hz, 1H), 7.99-7.92 (m, 1H), 7.91-7.80 (m, 2H), 7.75 (d, J=6.4 Hz, 1H), 7.61-7.53 (m, 1H), 7.49 (d, J=2.4 Hz, 1H), 7.38 (t, J=5.6 Hz, 1H), 7.28-7.14 (m, 2H), 4.98 (d, J=7.2 Hz, 1H), 4.76 (t, J=8.4 Hz, 2H), 4.46-4.40 (m, 1H), 4.39 (s, 2H), 4.37-4.32 (m, 1H), 4.08-3.96 (m, 3H), 3.82-3.73 (m, 3H), 3.68 (s, 2H), 3.65-3.55 (m, 2H), 3.55-3.46 (m, 4H), 3.43-3.38 (m, 1H), 2.98-2.85 (m, 2H), 2.61-2.39 (m, 2H), 2.02-1.90 (m, 3H), 1.89-1.77 (m, 5H), 1.76-1.58 (m, 7H), 1.57-1.49 (m, 1H), 1.35-1.07 (m, 6H), 0.93-0.80 (m, 4H). ¹⁹F NMR (376 MHz, METHANOL-d₄) δ=−118.137, −120.742.

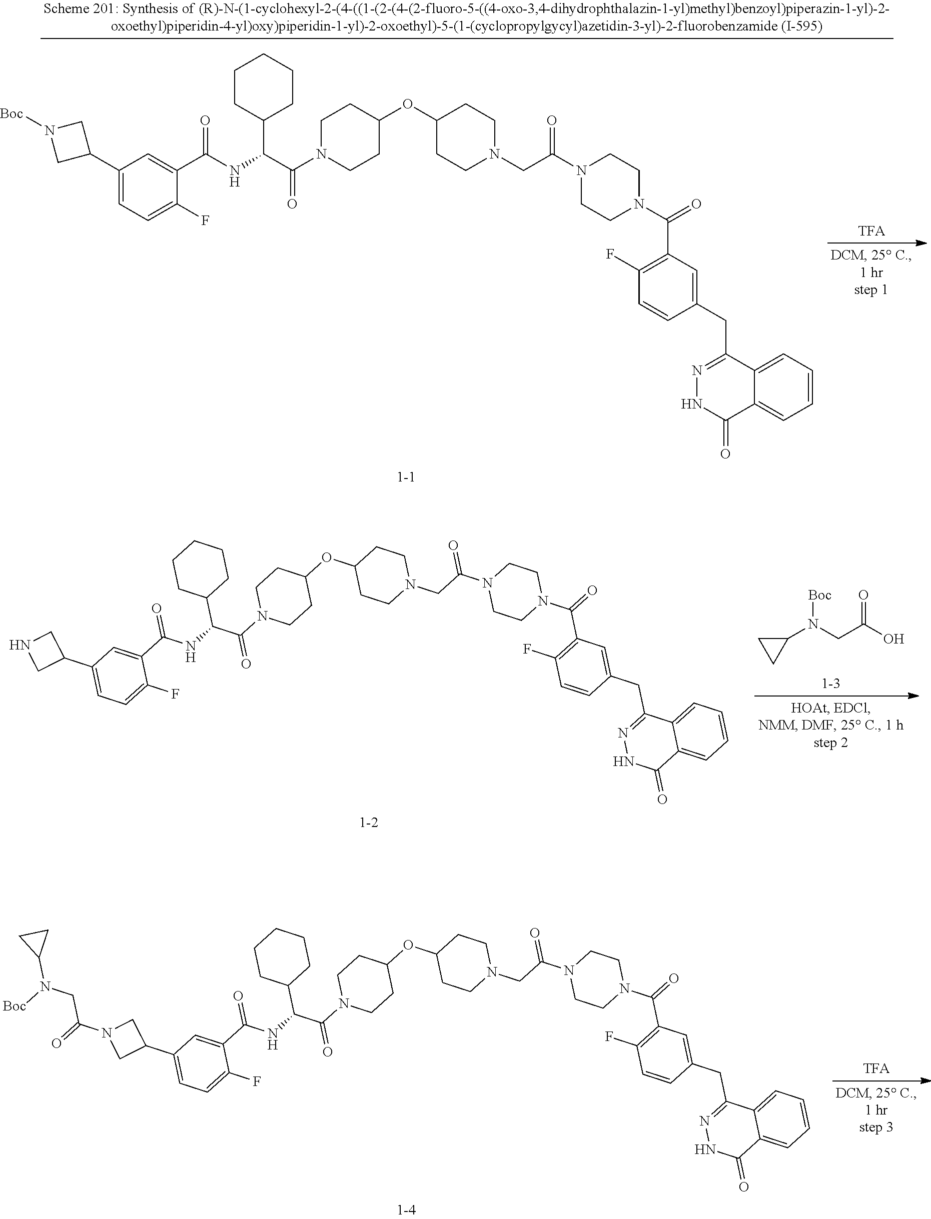 [SMILES: CC(C)(C)OC(=O)N1CC(c2ccc(F)c(C(=O)N[C@@H](C(=O)N3CCC(OC4CCN(CC(=O)N5CCN(C(=O)c6cc(Cc7n[nH]c(=O)c8ccccc78)ccc6F)CC5)CC4)CC3)C3CCCCC3)c2)C1 ; O=C(N[C@@H](C(=O)N1CCC(OC2CCN(CC(=O)N3CCN(C(=O)c4cc(Cc5n[nH]c(=O)c6ccccc56)ccc4F)CC3)CC2)CC1)C1CCCCC1)c1cc(C2CNC2)ccc1F ; CC(C)(C)OC(=O)N(CC(=O)O)C1CC1 ; CC(C)(C)OC(=O)N(CC(=O)N1CC(c2ccc(F)c(C(=O)N[C@@H](C(=O)N3CCC(OC4CCN(CC(=O)N5CCN(C(=O)c6cc(Cc7n[nH]c(=O)c8ccccc78)ccc6F)CC5)CC4)CC3)C3CCCCC3)c2)C1)C1CC1]

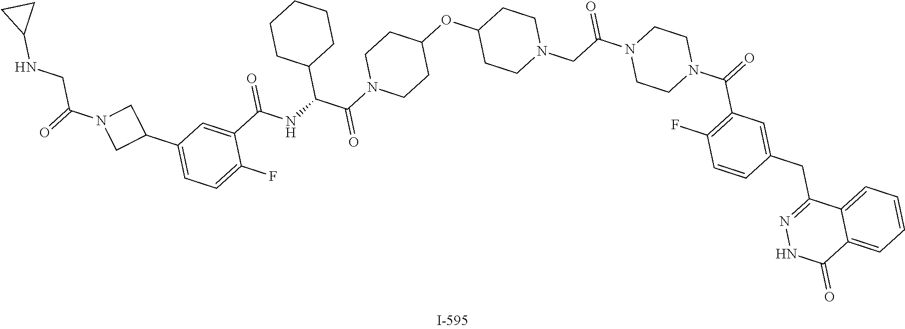 [SMILES: O=C(N[C@@H](C(=O)N1CCC(OC2CCN(CC(=O)N3CCN(C(=O)c4cc(Cc5n[nH]c(=O)c6ccccc56)ccc4F)CC3)CC2)CC1)C1CCCCC1)c1cc(C2CN(C(=O)CNC3CC3)C2)ccc1F]

### Step 1: Synthesis of Intermediate 1-2

To a solution of Intermediate 1-1 (80 mg, 79.43 μmol, 1 eq) in DCM (1 mL) was added TFA (307.00 mg, 2.69 mmol, 0.2 mL, 33.90 eq). The mixture was stirred at 25° C. for 1 hr. The reaction mixture was concentrated under reduced pressure to give a residue. The crude was used in the next step. Intermediate 1-2 (80 mg, 78.35 μmol, 98.64% yield, TFA salt) was obtained as a white solid. LCMS (Method D): Retention time: 0.314 min, [M+H]⁺=907.3.

### Step 2: Synthesis of Intermediate 1-4

To a solution of Intermediate 1-2 (80 mg, 78.35 μmol, 1 eq, TFA salt) and Intermediate 1-3 (16.86 mg, 78.35 μmol, 1 eq) in DMF (0.8 mL) was added EDCI (30.04 mg, 156.70 μmol, 2 eq), HOAt (10.66 mg, 78.35 μmol, 10.96 μL, 1 eq) and NMM (39.62 mg, 391.74 μmol, 43.07 μL, 5 eq). The mixture was stirred at 25° C. for 1 hr. To the reaction mixture was added H₂O (2 mL) and then it was extracted with DCM (2 mL*3), the combined organic phase was washed, with dried by Na₂SO₄, filtered and concentrated. The crude product was purified by reversed-phase HPLC (0.1% TFA condition), and the eluent was concentrated to remove ACN and lyophilized to Intermediate 1-4 (80 mg, 72.44 μmol, 92.47% yield) as a white solid. LCMS (Method D): Retention time: 0.409 min, [M+H]⁺=1104.4. SFC: Retention time: 1.830 min

### Step 3: Synthesis of I-595

To a solution of Intermediate 1-4 (70 mg, 63.39 μmol, 1 eq) in DCM (1 mL) was added TFA (307.00 mg, 2.69 mmol, 0.2 mL, 42.47 eq). The mixture was stirred at 25° C. for 1 hr. The reaction mixture was concentrated under reduced pressure to give a residue. To the reaction mixture was added H₂O (10 ml) and ACN (2 ml) and it was lyophilized to give I-595 (60.15 mg, 53.00 μmol, 83.61% yield, 98.522% purity, TFA salt) as a white solid. LCMS (Method D): Retention time: 0.320 min, (M+H)=1004.5. SFC: Retention time: 1.927 min. ¹H NMR (400 MHz, METHANOL-d₄) δ=8.37 (d, J=7.6 Hz, 1H), 8.28 (t, J=5.6 Hz, 1H), 7.99-7.92 (m, 1H), 7.91-7.79 (m, 2H), 7.75 (t, J=5.2 Hz, 1H), 7.57 (d, J=3.2 Hz, 1H), 7.53-7.50 (m, 1H), 7.42-7.32 (m, 1H), 7.30-7.21 (m, 1H), 7.18 (t, J=9.2 Hz, 1H), 4.98 (d, J=4.0 Hz, 2H), 4.66 (t, J=8.4 Hz, 1H), 4.56-4.46 (m, 1H), 4.42-4.35 (m, 2H), 4.31 (d, J=4.8 Hz, 2H), 4.23 (d, J=5.2 Hz, 1H), 4.14-4.04 (m, 2H), 4.04-3.98 (m, 3H), 3.96 (s, 1H), 3.91-3.75 (m, 4H), 3.73 (d, J=2.0 Hz, 1H), 3.69-3.62 (m, 1H), 3.56 (s, 1H), 3.52-3.46 (m, 2H), 3.46-3.41 (m, 1H), 3.36 (s, 3H), 3.20-3.07 (m, 1H), 2.85-2.77 (m, 1H), 2.28-2.14 (m, 1H), 2.12-1.93 (m, 4H), 1.91-1.76 (m, 5H), 1.75-1.63 (m, 3H), 1.61-1.51 (m, 1H), 1.33-1.10 (m, 5H), 0.98-0.86 (m, 5H). ¹⁹F NMR (376 MHz, METHANOL-d₄) δ=−77.302, −117.738, −120.706.

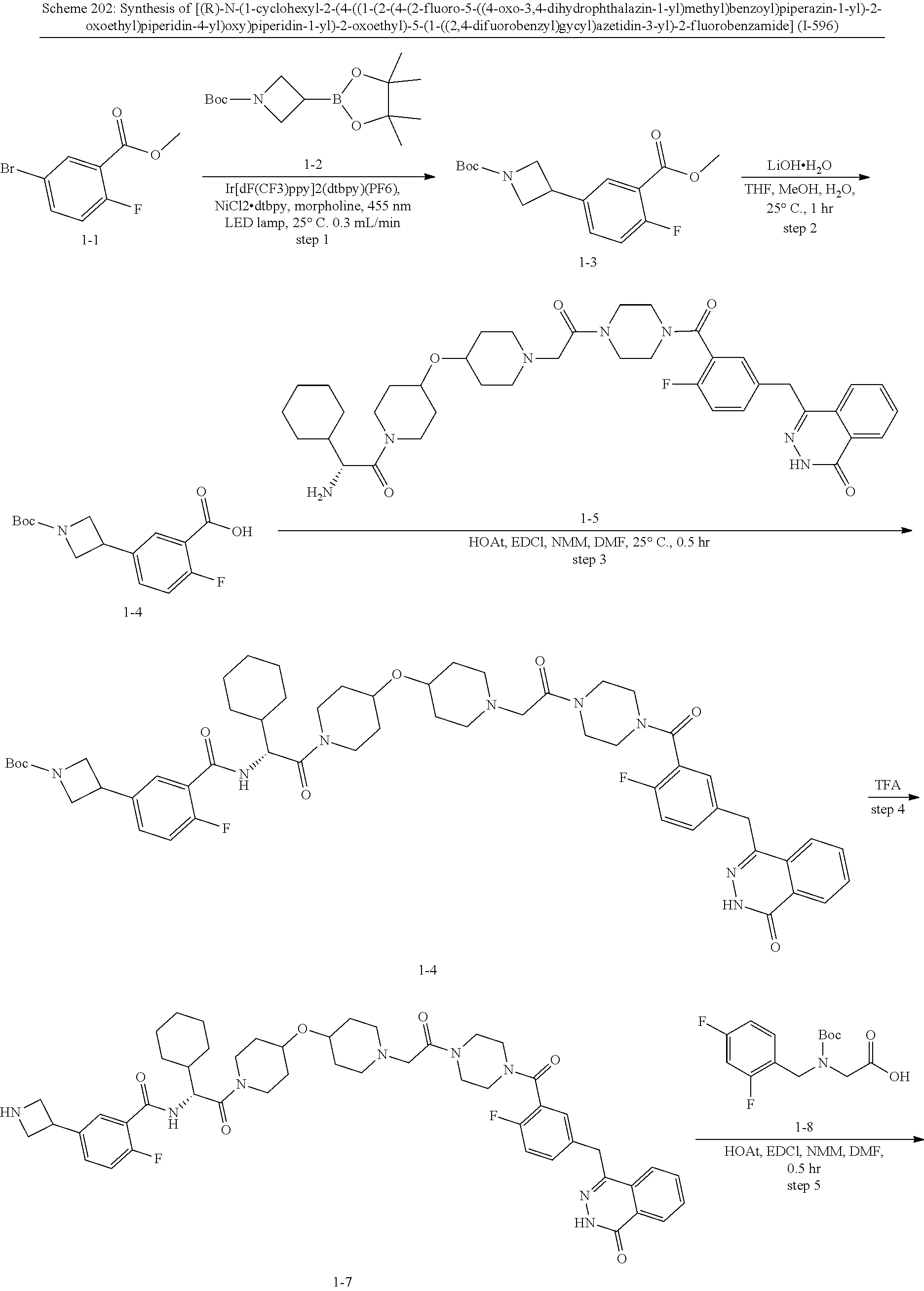

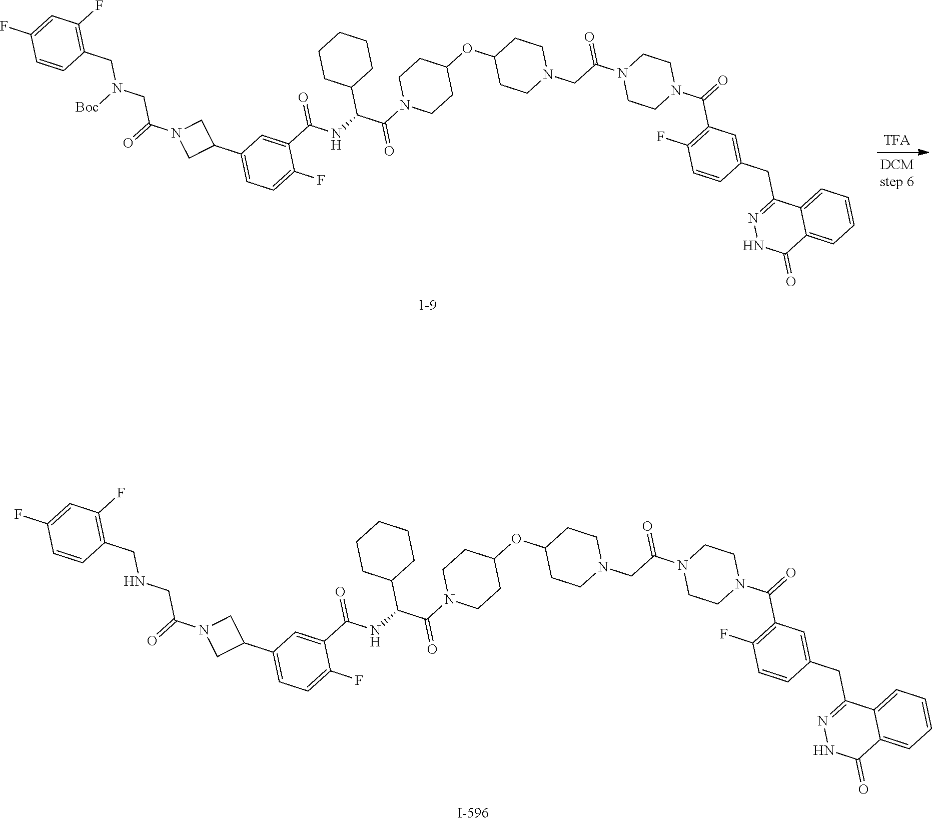 [SMILES: CC(C)(C)OC(=O)N(CC(=O)N1CC(c2ccc(F)c(C(=O)N[C@@H](C(=O)N3CCC(OC4CCN(CC(=O)N5CCN(C(=O)c6cc(Cc7n[nH]c(=O)c8ccccc78)ccc6F)CC5)CC4)CC3)C3CCCCC3)c2)C1)Cc1ccc(F)cc1F ; O=C(N[C@@H](C(=O)N1CCC(OC2CCN(CC(=O)N3CCN(C(=O)c4cc(Cc5n[nH]c(=O)c6ccccc56)ccc4F)CC3)CC2)CC1)C1CCCCC1)c1cc(C2CN(C(=O)CNCc3ccc(F)cc3F)C2)ccc1F]

### Step 1: Synthesis of Intermediate 1-3

To a 15 mL vial equipped with a stir bar was added Intermediate 1-1 (164.59 mg, 706.29 μmol, 1 eq), Intermediate 1-2 (0.4 g, 1.41 mmol, 2 eq), Ir[dF(CF₃)ppy]₂(dtbpy)(PF₆) (7.92 mg, 7.06 μmol, 0.01 eq), NiCl₂-dtbpy (14.05 mg, 35.31 μmol, 0.05 eq), and morpholine (92.30 mg, 1.06 mmol, 93.23 μL, 1.5 eq) in DMF (4 mL). The reaction solution was pumped through the reactor at a flow rate of 300 L min⁻¹, irradiating with a 455 nm LED lamp, with cooling water to keep the reaction temperature at 25° C. for 1 h. The reaction mixture was concentrated under reduced pressure to give a residue. The residue was purified by reversed-phase HPLC (0.1% FA condition), the eluent was concentrated and lyophilized to give Intermediate 1-3 (0.25 g, crude) as a white solid. LCMS (Method D): Retention time: 0.504 min, [M+H]⁺=254.1.

### Step 2: Synthesis of Intermediate 1-4

To a solution of Intermediate 1-3 (40 mg, 129.31 μmol, 1 eq) in THF (0.2 mL), MeOH (0.2 mL), and H₂O (0.2 mL) was added LiOH·H₂O (16.28 mg, 387.93 μmol, 3 eq). The mixture was stirred at 25° C. for 0.5 hr. The reaction mixture was adjusted to pH 6 by 1 M HCl, and then extracted with DCM (5 mL*2). The combined organic layers were washed with brine (3 mL*2), dried over Na₂SO₄, filtered and concentrated under reduced pressure to give Intermediate 1-4 (40 mg, crude) as a white solid. LCMS (Method D): Retention time: 0.429 min, [M+H]⁺=240.1.

### Step 3: Synthesis of Intermediate 1-6

To a solution of Intermediate 1-4 (40 mg, 135.45 μmol, 1 eq) and Intermediate 1-5 (103.80 mg, 135.45 μmol, 1 eq, HCl salt) in DMF (1 mL) was added EDCI (77.90 mg, 406.36 μmol, 3 eq), HOAt (18.44 mg, 135.45 μmol, 18.95 μL, 1 eq), and NMM (68.50 mg, 677.26 μmol, 74.46 μL, 5 eq). The mixture was stirred at 25° C. for 0.5 hr. The mixture was filtered and concentrated under reduced pressure to give a residue. The residue was purified by reversed-phase HPLC (0.1% FA condition), the eluent was concentrated and lyophilized to give Intermediate 1-6 (0.06 g, 59.57 μmol, 43.98% yield) as a white solid. LCMS: Retention time (Method D): 0.475 min, [M+H]⁺=1007.5. SFC: Retention time: 1.355 min.

### Step 4: Synthesis of Intermediate 1-7

To a solution of Intermediate 1-6 (50 mg, 49.64 μmol, 1 eq) in DCM (1 mL) was added TFA (307.00 mg, 2.69 mmol, 0.2 mL, 54.23 eq). The mixture was stirred at 25° C. for 0.15 hr. The reaction mixture was concentrated under reduced pressure to give Intermediate 1-7 (50 mg, crude, TFA salt). LCMS (Method D): Retention time: 0.314 min, [M+H]⁺=907.3. SFC: Retention time: 1.088 min.

### Step 5: Synthesis of Intermediate 1-9

To a solution of Intermediate 1-7 (0.05 g, 48.97 μmol, 1 eq, TFA salt) and Intermediate 1-8 (14.75 mg, 48.97 μmol, 1 eq) in DMF (0.5 mL) was added EDCI (28.16 mg, 146.90 μmol, 3 eq), HOAt (6.67 mg, 48.97 μmol, 6.85 μL, 1 eq) and NMM (24.76 mg, 244.84 μmol, 26.92 μL, 5 eq). The mixture was stirred at 25° C. for 0.5 hr. The reaction mixture was concentrated under reduced pressure to give a residue. The residue was purified by reversed-phase HPLC (0.1% FA condition), the eluent was concentrated to remove ACN and lyophilized to give Intermediate 1-9 (30 mg, 25.20 μmol, 51.47% yield) as a white solid. LCMS (Method D): Retention time: 0.432 min, [M+H]⁺=1190.6. SFC: Retention time: 0.807 min.

### Step 6: Synthesis of I-596

To a solution of Intermediate 1-9 (20 mg, 16.80 μmol, 1 eq) in DCM (0.5 mL) was added TFA (153.50 mg, 1.35 mmol, 0.1 mL, 80.12 eq). The mixture was stirred at 25° C. for 0.15 hr. The reaction mixture was concentrated under reduced pressure to give I-596 (12.87 mg, 10.15 μmol, 60.38% yield, 94.927% purity, TFA salt) as a white solid. LCMS (Method D): Retention time: 0.338 min, [M+H]⁺=1090.3. SFC: Retention time: 0.909 min. ¹H NMR (400 MHz, METHANOL-d₄) δ=8.37 (d, J=7.6 Hz, 1H), 8.30-8.27 (m, 1H), 7.99-7.91 (m, 1H), 7.91-7.80 (m, 2H), 7.79-7.71 (m, 1H), 7.64-7.55 (m, 2H), 7.51 (s, 1H), 7.40-7.32 (m, 1H), 7.28-7.23 (m, 1H), 7.20-7.07 (m, 3H), 5.02-4.96 (m, 1H), 4.67-4.58 (m, 1H), 4.54-4.45 (m, 1H), 4.39 (s, 2H), 4.34-4.29 (m, 3H), 4.28-4.20 (m, 2H), 4.13-3.99 (m, 3H), 3.94 (s, 3H), 3.87-3.70 (m, 5H), 3.68-3.40 (m, 7H), 2.26-2.16 (m, 1H), 2.11-2.01 (m, 3H), 1.99-1.92 (m, 1H), 1.92-1.75 (m, 6H), 1.69 (s, 3H), 1.64-1.49 (m, 2H), 1.33 (s, 2H), 1.27-1.06 (m, 5H). ¹⁹F NMR (376 MHz, METHANOL-d₄) δ=−77.107, −108.244, −112.912, −117.723, −120.713.

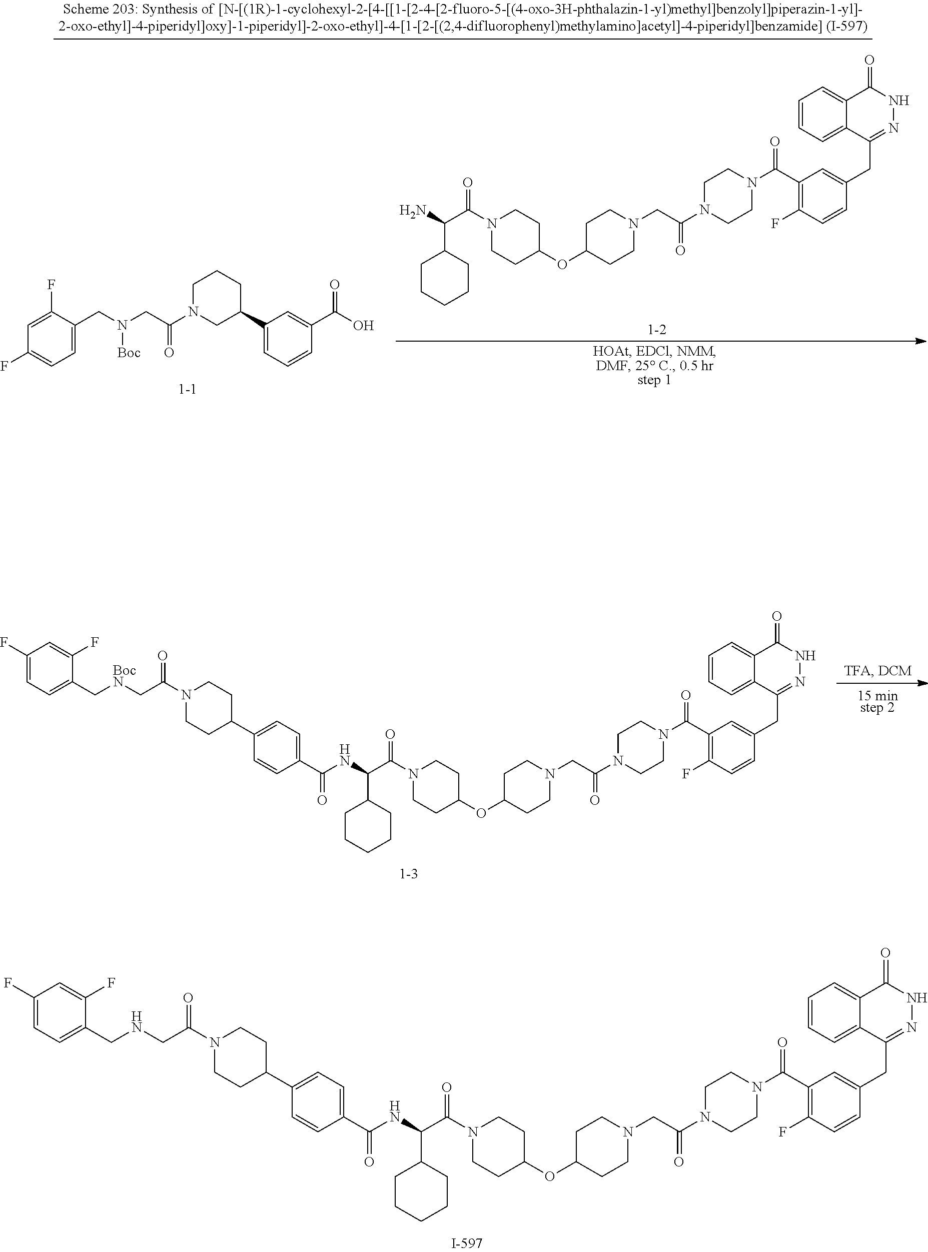

### Step 1: Synthesis of Intermediate 1-3

To a solution of Intermediate 1-2 (100 mg, 104.87 μMol, 1 eq, HCl salt) in DMF (1 mL) was added EDCI (60.31 mg, 314.60 μmol, 3 eq), NMM (53.04 mg, 524.34 μmol, 57.65 μL, 5 eq), Intermediate 1-1 (31.60 mg, 104.87 μmol, 1 eq) and HOAt (14.27 mg, 104.87 μmol, 14.67 μL, 1 eq). The mixture was stirred at 25° C. for 0.5 h. The reaction mixture was diluted with H₂O (2 mL) and extracted with EA (2 mL*3). The combined organic layers were washed with brine (2 mL*3), dried over Na₂SO₄, filtered and concentrated under reduced pressure to give a residue. The crude product was purified by prep-HPLC (column: CD01-Phenomenex luna C18 150*25*10 um; mobile phase: [water(FA)-ACN]; gradient: 31%-61% B over 10 min) and the eluent was lyophilized to give Intermediate 1-3 (90 mg, 74.23 μmol, 70.78% yield, 99% purity) as a white solid. LCMS (Method D): Retention time: 0.507 min, [M+H]⁺=1200.7. SFC: Retention time: 0.808 min.

### Step 2: Synthesis of I-597

To a solution of Intermediate 1-3 (80 mg, 66.65 μmol, 1 eq) in DCM (0.6 mL) was added TFA (307.00 mg, 2.69 mmol, 0.2 mL, 40.40 eq). The mixture was stirred at 25° C. for 15 min. The mixture was concentrated under reduced pressure to give a residue. The crude product was purified by reversed phase HPLC (0.1% TFA condition) and the eluent was lyophilized to give I-597 (29.81 mg, 24.39 μmol, 36.60% yield, 99.367% purity, TFA salt) as a white solid. SFC: Retention time: 3.154 min, 5.482 min. LCMS (Method D): Retention time: 0.393 min, (M+H)=1100.6. ¹H NMR (400 MHz, METHANOL-d₄) δ=8.37 (d, J=7.6 Hz, 1H), 7.99-7.91 (m, 1H), 7.91-7.76 (m, 4H), 7.67-7.57 (m, 1H), 7.51 (d, J=3.2 Hz, 1H), 7.36 (d, J=8.0 Hz, 3H), 7.21-7.06 (m, 3H), 4.92 (d, J=8.8 Hz, 1H), 4.66 (d, J=13.2 Hz, 1H), 4.39 (s, 2H), 4.34-4.28 (m, 3H), 4.26-4.19 (m, 2H), 4.17-4.12 (m, 1H), 4.04-4.02 (m, 1H), 3.95 (s, 1H), 3.81 (s, 5H), 3.73-3.61 (m, 2H), 3.60-3.41 (m, 5H), 3.36 (s, 3H), 3.29-3.21 (m, 2H), 3.19-3.05 (m, 1H), 3.00-2.89 (m, 1H), 2.85 (t, J=12.0 Hz, 1H), 2.28-2.13 (m, 1H), 2.05 (s, 2H), 2.00-1.82 (m, 7H), 1.76 (d, J=7.6 Hz, 2H), 1.73-1.51 (m, 6H), 1.34-1.19 (m, 3H), 1.16-1.05 (m, 2H). ¹⁹F NMR (376 MHz, METHANOL-d₄) δ=−77.221, −108.394, −112.905, −120.665, −120.740.

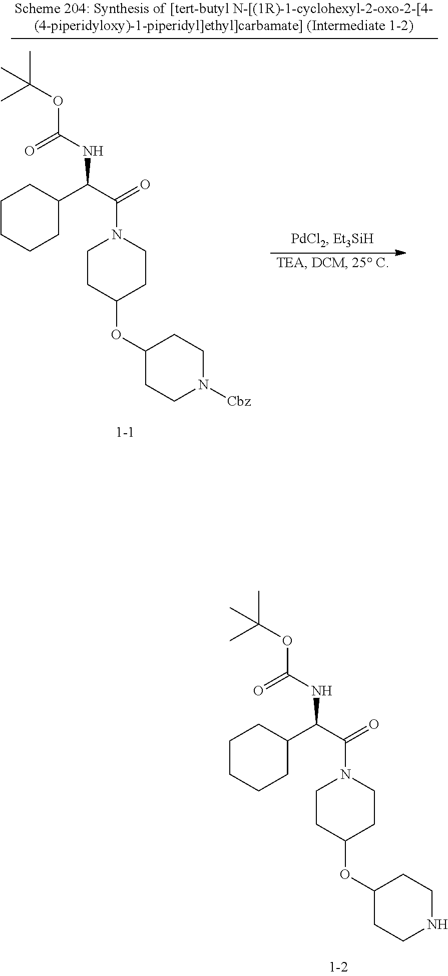

To a solution of Intermediate 1-1 (100 mg, 179.30 μmol, 1 eq) in DCM (1 mL) was added PdCl₂ (15.90 mg, 89.65 μmol, 0.5 eq), Et₃SiH (250.18 mg, 2.15 mmol, 343.66 μL, 12 eq) and TEA (54.43 mg, 537.90 μmol, 74.87 μL, 3 eq). The mixture was stirred at 25° C. for 0.5 h. The mixture was filtered and concentrated under reduced pressure to give a residue. The crude product was purified by prep-HPLC (column: CD04-Welch Utimate C18 150*25*7 um; mobile phase: [water(FA)-ACN]; gradient: 15%-45% B over 8 min) and the eluent was lyophilized to give Intermediate 1-2 (21.53 mg, 44.18 μmol, 24.64% yield, 96.372% purity, FA salt) as a white solid. LCMS (Method D): Retention time: 0.378 min, [M+H]⁺=424.5. SFC: Retention time: 0.616 min, 0.720 min. ¹H NMR (400 MHz, METHANOL-d₄) δ=8.53 (s, 1H), 4.38 (d, J=8.0 Hz, 1H), 4.06-3.89 (m, 1H), 3.88-3.73 (m, 3H), 3.58-3.34 (m, 2H), 3.31-3.19 (m, 2H), 3.17-3.05 (m, 2H), 2.07-1.95 (m, 2H), 1.95-1.80 (m, 4H), 1.76 (d, J=9.2 Hz, 3H), 1.69-1.57 (m, 4H), 1.55-1.48 (m, 1H), 1.44 (d, J=2.0 Hz, 9H), 1.30-1.15 (m, 3H), 1.11-0.96 (m, 2H).

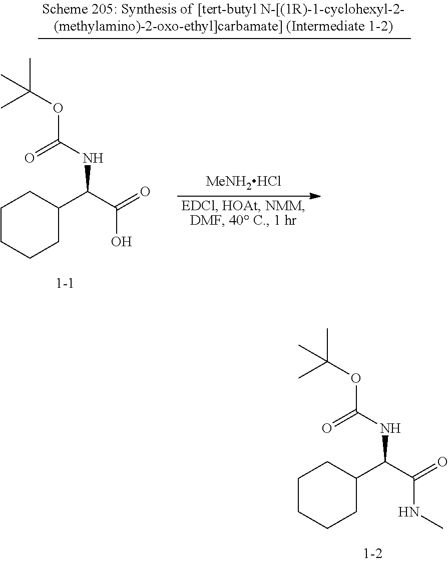 [SMILES: "MeNH2·HCl" ; CC(C)(C)OC(=O)N[C@@H](C(=O)O)C1CCCCC1 ; "EDCl, HOAt, NMM,DMF, 40° C., 1 hr" ; CNC(=O)[C@H](NC(=O)OC(C)(C)C)C1CCCCC1]

### Step 1: Synthesis of 1-2.

To a solution of Intermediate 1-1 (400 mg, 1.55 mmol, 1 eq) in DMF (4 mL) was added methanamine;hydrochloride (125.94 mg, 1.87 mmol, 1.2 eq), EDCI (893.97 mg, 4.66 mmol, 3 eq), HOAt (211.58 mg, 1.55 mmol, 217.45 μL, 1 eq) and NMM (786.14 mg, 7.77 mmol, 854.50 μL, 5 eq). The mixture was stirred at 25° C. for 1 hr. The mixture was concentrated under reduced pressure to give a residue. The crude product was purified by prep-HPLC (column: CD04-Welch Utimate C18 150*25*7 um; mobile phase: [water(FA)-ACN]; gradient: 25%-55% B over 8 min), the eluent was concentrated and lyophilized to give Intermediate 1-2 (164.57 mg, 565.60 μmol, 36.39% yield, 92.920% purity) as a white solid. LCMS (Method D): Retention time: 0.421 min, [M+H]⁺=271.2. SFC: Retention time: 0.837 min, 0.909 min. ¹H NMR (400 MHz, METHANOL-d₄) δ=3.81 (d, J=5.2 Hz, 1H), 2.73 (s, 3H), 1.81-1.53 (m, 6H), 1.44 (s, 9H), 1.31-1.16 (m, 3H), 1.15-0.97 (m, 2H).

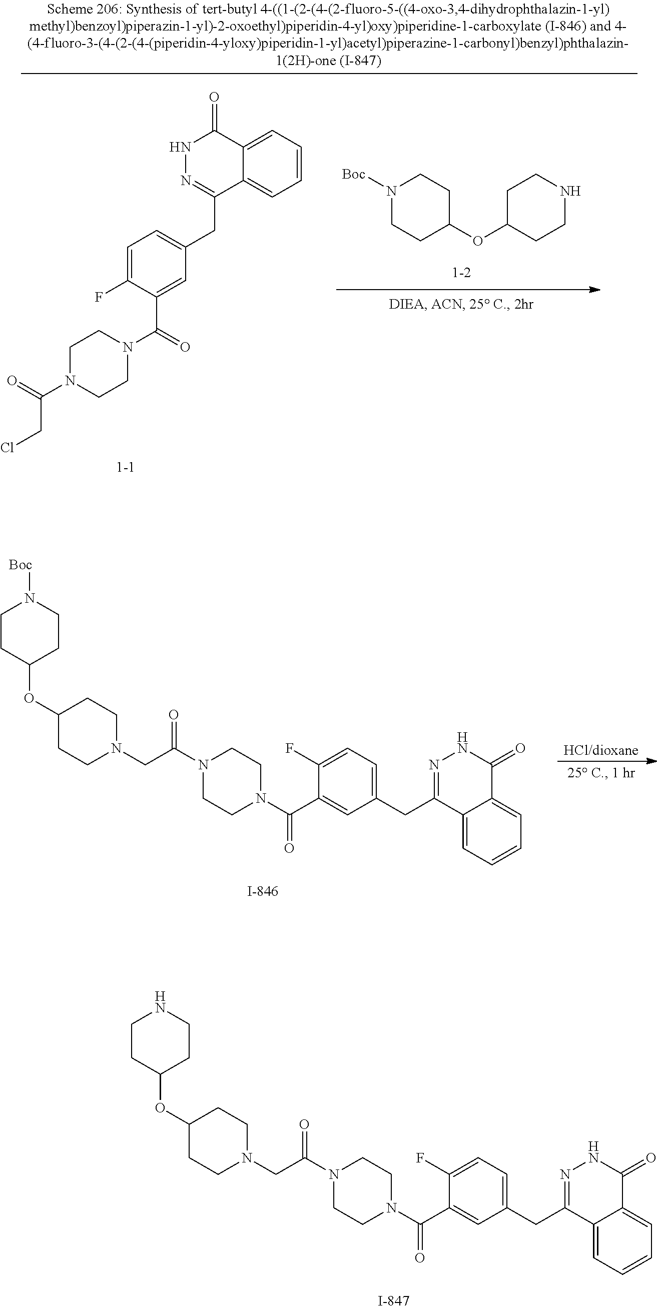

### Step 1: Synthesis of I-846

To a solution of Intermediate 1-1 (100 mg, 225.80 μmol, 1 eq) and Intermediate 1-2 (64.22 mg, 225.80 μmol, 1 eq) in ACN (1 mL) was added DIEA (29.18 mg, 225.80 μmol, 39.33 μL, 1 eq). The mixture was stirred at 25° C. for 1 hr. The reaction mixture was concentrated under reduced pressure to give a residue. The residue was purified by prep-HPLC (column: CD01-Phenomenex luna C18 150*25*10 um; mobile phase: [water (FA)-ACN]; gradient: 15%-45% B over 10 min) and the eluent was concentrated to remove ACN and lyophilized to give I-846 (74.69 mg, 100.72 μmol, 44.61% yield, 99.364% purity, FA salt) as a white solid. LCMS (Method D): Retention time: 0.323 min, (M+H)=691.3. ¹H NMR (400 MHz, METHANOL-d₄) δ=8.43 (s, 1H), 8.37 (d, J=7.6 Hz, 1H), 7.94 (s, 1H), 7.91-7.79 (m, 2H), 7.50 (s, 1H), 7.37 (s, 1H), 7.17 (t, J=8.8 Hz, 1H), 4.39 (s, 2H), 3.84-3.59 (m, 10H), 3.53 (s, 1H), 3.47 (s, 3H), 3.21-3.01 (m, 4H), 2.87-2.64 (m, 2H), 2.05-1.90 (m, 2H), 1.80 (s, 4H), 1.45 (s, 11H). ¹⁹F NMR (376 MHz, METHANOL-d₄) δ=−120.779.

### Step 2: Synthesis of I-847

I-846 (50 mg, 67.86 μmol, 1 eq, FA salt) was added into HCl/dioxane (2 M, 468.77 μL, 13.82 eq) and then the mixture was stirred at 25° C. for 1 hr. The reaction mixture was concentrated under reduced pressure to give a residue. The residue was purified by prep-HPLC (column: CD01-Phenomenex luna C18 150*25*10 um; mobile phase: [water (FA)-ACN]; gradient: 0%-29% B over 10 min) the eluent was concentrated to remove ACN and lyophilized to give I-847 (14.64 mg, 22.60 μmol, 33.30% yield, 98.276% purity, FA salt) as a white solid. LCMS (Method D): Retention time: 0.216 min, (M+H)=591.2. ¹H NMR (400 MHz, METHANOL-d₄) δ=8.41 (s, 1H), 8.37 (d, J=7.2 Hz, 1H), 7.95 (s, 1H), 7.91-7.78 (m, 2H), 7.50 (s, 1H), 7.37 (s, 1H), 7.17 (t, J=8.8 Hz, 1H), 4.39 (s, 2H), 3.81 (s, 2H), 3.75 (s, 1H), 3.63 (d, J=13.2 Hz, 4H), 3.56-3.45 (m, 3H), 3.39-3.32 (m, 4H), 3.18-3.06 (m, 2H), 3.06-2.90 (m, 2H), 2.74-2.54 (m, 2H), 2.08-1.90 (m, 4H), 1.88-1.63 (m, 4H). ¹⁹F NMR (376 MHz, METHANOL-d₄) δ=−120.802.

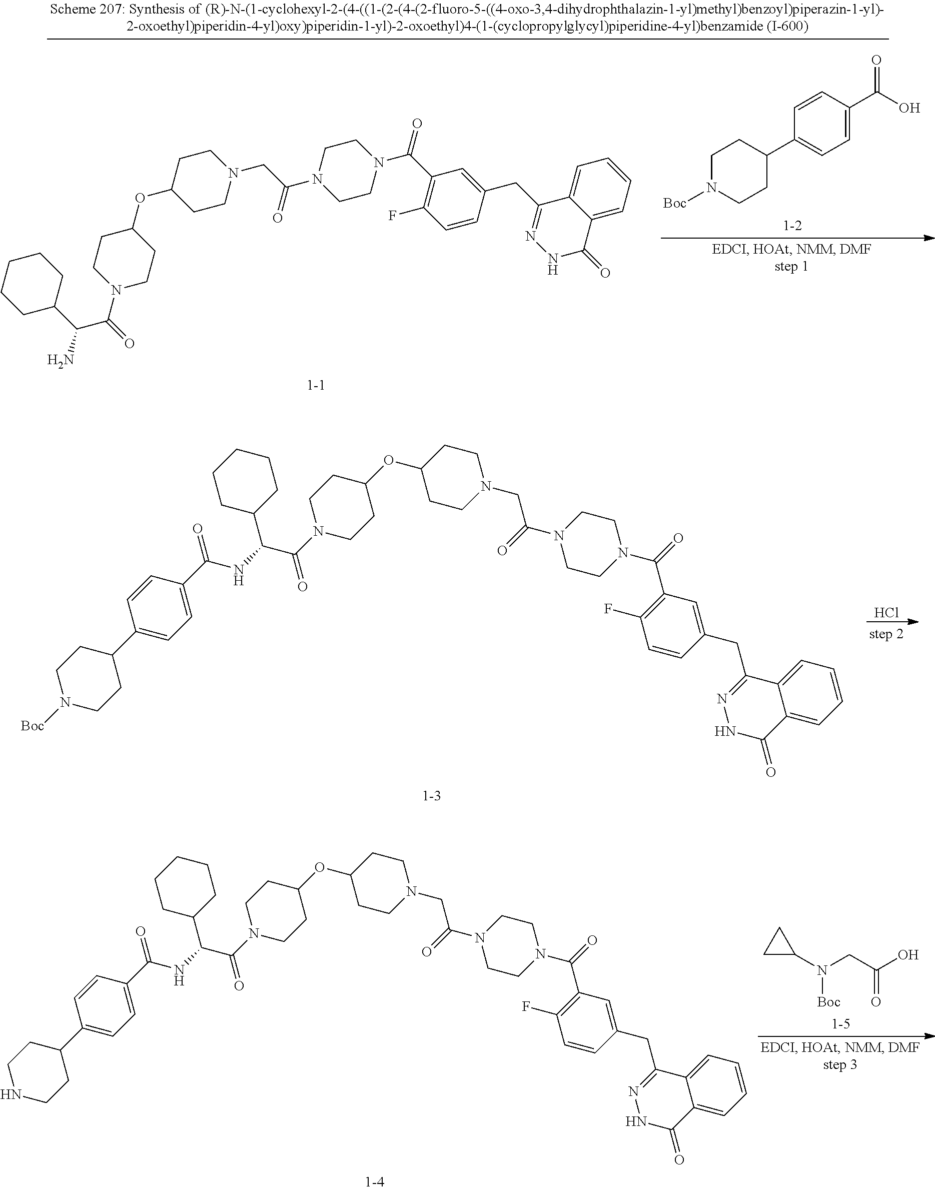 [SMILES: N[C@@H](C(=O)N1CCC(OC2CCN(CC(=O)N3CCN(C(=O)c4cc(Cc5n[nH]c(=O)c6ccccc56)ccc4F)CC3)CC2)CC1)C1CCCCC1 ; CC(C)(C)OC(=O)N1CCC(c2ccc(C(=O)O)cc2)CC1 ; CC(C)(C)OC(=O)N1CCC(c2ccc(C(=O)N[C@@H](C(=O)N3CCC(OC4CCN(CC(=O)N5CCN(C(=O)c6cc(Cc7n[nH]c(=O)c8ccccc78)ccc6F)CC5)CC4)CC3)C3CCCCC3)cc2)CC1 ; Cl ; O=C(N[C@@H](C(=O)N1CCC(OC2CCN(CC(=O)N3CCN(C(=O)c4cc(Cc5n[nH]c(=O)c6ccccc56)ccc4F)CC3)CC2)CC1)C1CCCCC1)c1ccc(C2CCNCC2)cc1 ; CC(C)(C)OC(=O)N(CC(=O)O)C1CC1]

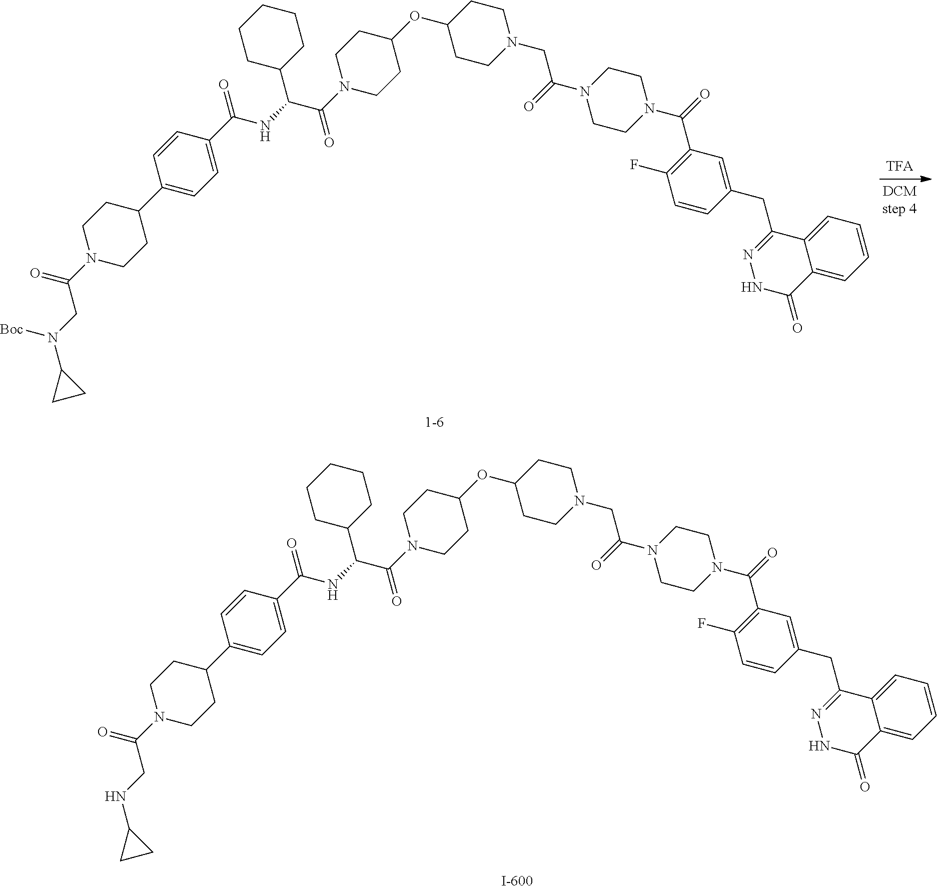 [SMILES: CC(C)(C)OC(=O)N(CC(=O)N1CCC(c2ccc(C(=O)N[C@@H](C(=O)N3CCC(OC4CCN(CC(=O)N5CCN(C(=O)c6cc(Cc7n[nH]c(=O)c8ccccc78)ccc6F)CC5)CC4)CC3)C3CCCCC3)cc2)CC1)C1CC1 ; O=C(N[C@@H](C(=O)N1CCC(OC2CCN(CC(=O)N3CCN(C(=O)c4cc(Cc5n[nH]c(=O)c6ccccc56)ccc4F)CC3)CC2)CC1)C1CCCCC1)c1ccc(C2CCN(C(=O)CNC3CC3)CC2)cc1]

### Step 1: Synthesis of Intermediate 1-3

To a solution of Intermediate 1-1 (400 mg, 521.96 μmol, 1 eq, HCl salt) and Intermediate 1-2 (159.39 mg, 521.96 μmol, 1 eq) in DMF (4 mL) was added EDCI (200.12 mg, 1.04 mmol, 2 eq) and HOAt (71.04 mg, 521.96 μmol, 73.02 μL, 1 eq) and NMM (263.97 mg, 2.61 mmol, 286.93 μL, 5 eq). The mixture was stirred at 25° C. for 12 hr. The reaction mixture was quenched by addition of H₂O (5 mL) and extracted with EA (5 mL*3), the combined organic layers were dried over Na₂SO₄, filtered and concentrated under reduced pressure to give a residue. The residue was purified by flash silica gel chromatography (ISCO; 40 g SepaFlash Silica Flash Column, Eluent of 0˜100% Ethyl acetate/Petroleum ether gradient @50 mL/min) and the eluent was concentrated to give Intermediate 1-3 (400 mg, 393.22 μmol, 75.34% yield, N/A purity) as a white solid. LCMS (Method D): Retention time: 0.417 min, [M+H]⁺=1017.5. SFC: Retention time: 1.847 min.

### Step 2: Synthesis of Intermediate 1-4

A solution of Intermediate 1-3 (370 mg, 363.73 μmol, 1 eq) in HCl/dioxane (2 M, 3.70 mL, 20.34 eq) was stirred at 25° C. for 1 hr. The reaction mixture was concentrated under reduced pressure to give a residue. The crude was used in the next step. Intermediate 1-4 (370 mg, crude, HCl salt) was obtained as a white solid. LCMS (Method D): Retention time: 0.316 min, [M+H]⁺=917.5. SFC: Retention time: 1.888 min.

### Step 3: Synthesis of Intermediate 1-6

To a solution of Intermediate 1-4 (100 mg, 104.87 μmol, 1 eq, HCl salt) and Intermediate 1-5 (22.57 mg, 104.87 μmol, 1 eq) in DMF (1 mL) was added EDCI (40.21 mg, 209.74 μmol, 2 eq), HOAt (14.27 mg, 104.87 μmol, 14.67 μL, 1 eq) and NMM (53.04 mg, 524.34 μmol, 57.65 μL, 5 eq). The mixture was stirred at 25° C. for 1 hr. To the reaction mixture was added H₂O (2 mL) and then it was extracted with DCM (2 mL*3), the combined organic phase was dried by Na₂SO₄, filtered and concentrated. The crude product was purified by reversed-phase HPLC (0.1% TFA condition) and the eluent was concentrated to remove ACN and lyophilized to give Intermediate 1-6 (110 mg, 98.71 μmol, 94.13% yield) as a white solid. LCMS (Method D): Retention time: 0.411 min, [M+H]⁺=1114.5.

### Step 4: Synthesis of I-600

To a solution of Intermediate 1-6 (100 mg, 89.74 μmol, 1 eq) in DCM (1 mL) was added TFA (460.50 mg, 4.04 mmol, 0.3 mL, 45.00 eq). The mixture was stirred at 25° C. for 1 hr. The reaction mixture was concentrated under reduced pressure to give a residue. The residue was purified by prep-HPLC (column: CD01-Phenomenex luna C18 150*25*10 um; mobile phase: [water (TFA)-ACN]; gradient: 20%-50% B over 10 min) the eluent was concentrated to remove ACN and lyophilized to give the product. I-600 (60.52 mg, 52.66 μmol, 58.68% yield, 98.175% purity, TFA salt) was obtained as a white solid. LCMS (Method D): Retention time: 0.375 min, [M+H]⁺=1014.5. SFC: Retention time: 1.888 min. ¹H NMR (400 MHz, METHANOL-d₄) δ=8.37 (d, J=7.6 Hz, 1H), 7.99-7.93 (m, 1H), 7.91-7.82 (m, 2H), 7.79 (d, J=8.4 Hz, 2H), 7.53-7.50 (m, 1H), 7.37 (d, J=8.4 Hz, 3H), 7.18 (t, J=9.2 Hz, 1H), 4.92 (d, J=8.0 Hz, 1H), 4.66 (d, J=13.2 Hz, 1H), 4.39 (s, 2H), 4.33-4.26 (m, 2H), 4.25-4.16 (m, 2H), 4.10-3.99 (m, 1H), 3.95 (s, 1H), 3.87 (d, J=14.8 Hz, 1H), 3.80 (d, J=10.4 Hz, 4H), 3.73 (s, 1H), 3.66 (d, J=1.2 Hz, 1H), 3.58-3.49 (m, 3H), 3.47-3.42 (m, 1H), 3.34 (d, J=10.0 Hz, 4H), 3.28-3.23 (m, 1H), 3.20-3.03 (m, 1H), 2.94 (t, J=12.0 Hz, 1H), 2.89-2.76 (m, 2H), 2.30-2.14 (m, 1H), 2.04 (d, J=6.4 Hz, 3H), 2.00-1.89 (m, 4H), 1.86 (d, J=11.2 Hz, 3H), 1.81-1.72 (m, 3H), 1.72-1.61 (m, 4H), 1.59-1.49 (m, 1H), 1.34-1.19 (m, 3H), 1.17-1.05 (m, 2H), 0.97-0.87 (m, 4H). ¹⁹F NMR (377 MHz, METHANOL-d₄) δ=−77.142, −120.712.

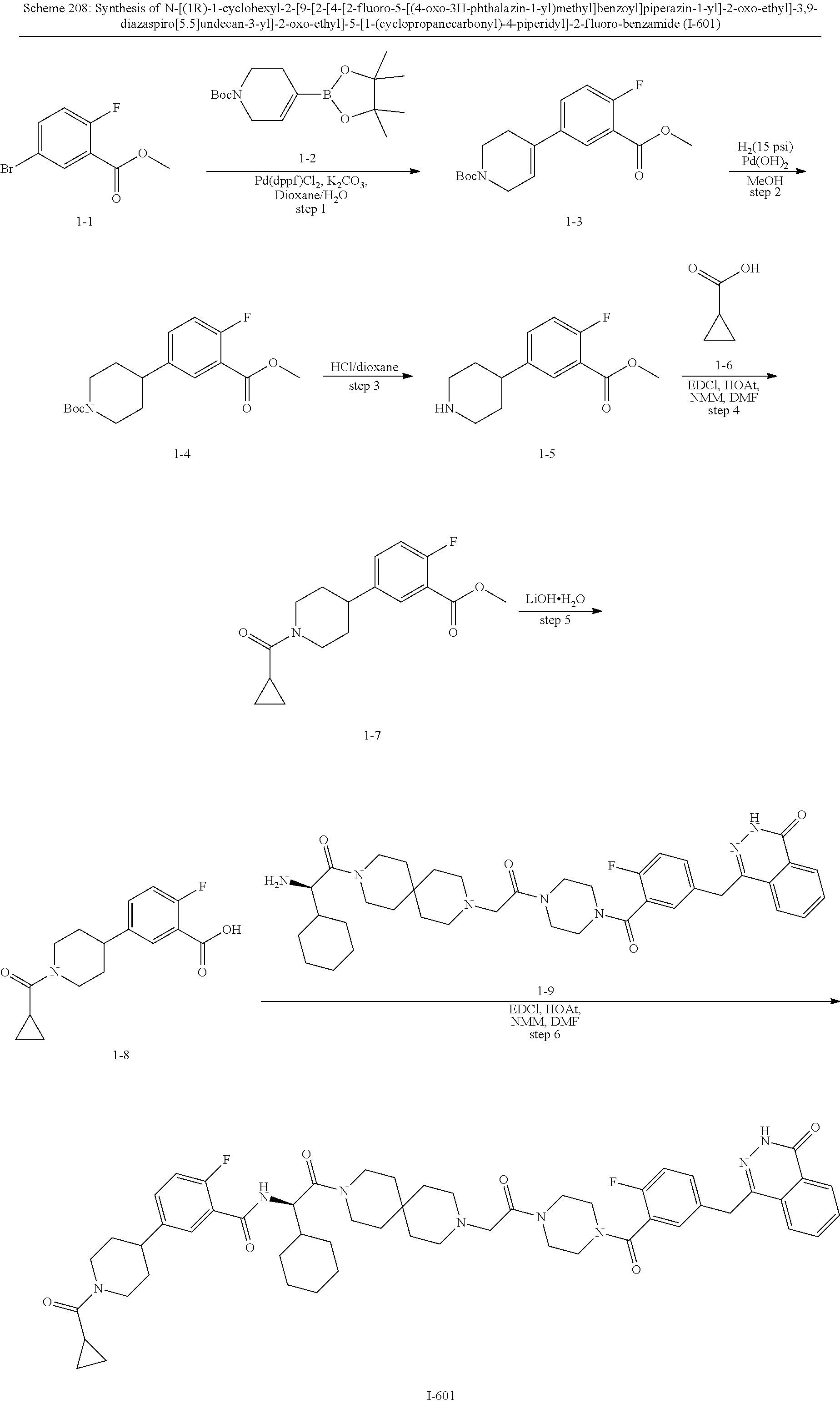

### Step 1: Synthesis of Intermediate 1-3

A mixture of Intermediate 1-1 (2 g, 8.58 mmol, 1 eq), Intermediate 1-2 (2.92 g, 9.44 mmol, 1.1 eq), Pd(dppf)Cl₂ (627.98 mg, 858.24 μmol, 0.1 eq) and K₂CO₃ (2.37 g, 17.16 mmol, 2 eq) in dioxane (20 mL) and H₂O (5 mL) was degassed and purged with N₂ 3 times, and then the mixture was stirred at 100° C. for 2 hr under N₂ atmosphere. The reaction mixture was quenched by H₂O (40 mL), and extracted with EA (20 mL*3). The combined organic layers were washed with brine (20 mL*3), dried over anhydrous Na₂SO₄, filtered and concentrated under reduced pressure to give a residue. The residue was purified by flash silica gel chromatography (ISCO; 40 g SepaFlash Silica Flash Column, Eluent of 0˜10% Ethyl acetate/Petroleum ether gradient @100 mL/min). The eluent was concentrated under vacuum. Intermediate 1-3 (1.3 g, 3.88 mmol, 45.17% yield) was obtained as light yellow oil. ¹H NMR (400 MHz, CHLOROFORM-d) δ=7.92 (dd, J=2.5, 6.9 Hz, 1H), 7.51 (ddd, J=2.5, 4.5, 8.6 Hz, 1H), 7.10 (dd, J=8.8, 10.3 Hz, 1H), 6.04 (br s, 1H), 4.08 (br d, J=2.4 Hz, 2H), 3.94 (s, 3H), 3.64 (t, J=5.6 Hz, 2H), 2.51 (br s, 2H), 1.49 (s, 9H).

### Step 2: Synthesis of Intermediate 1-4

To a solution of Pd(OH)₂/C (600 mg, 854.46 μmol, 20% purity, 0.22 eq) in MeOH (10 mL) was added Intermediate 1-3 (1.3 g, 3.88 mmol, 1 eq) in MeOH (10 mL). The mixture was degassed and purged with H₂ 3 times, and then the mixture was stirred at 60° C. for 2 h under H₂ (15 psi). The mixture was filtered and the filtrate was concentrated under vacuum to give a crude product. The crude was used for the next step directly without purification. Intermediate 1-4 (1.1 g, crude) was obtained as yellow oil. LCMS (Method G): Rt=0.735 min, [M−99]⁺=238.2.

### Step 3: Synthesis of Intermediate 1-5

To a solution of Intermediate 1-4 (1.1 g, 3.26 mmol, 1 eq) in DCM (6 mL) was added HCl/dioxane (4 M, 2.75 mL, 3.37 eq). The mixture was stirred at 25° C. for 1 h. The mixture was concentrated under vacuum to give a crude product. The crude was used for the next step directly without purification. Intermediate 1-5 (1 g, crude, HCl salt) was obtained as a yellow solid.

### Step 4: Synthesis of Intermediate 1-7

To a solution of Intermediate 1-5 (1 g, 3.65 mmol, 1 eq, HCl salt) in DMF (10 mL) was added HOAt (497.24 mg, 3.65 mmol, 511.04 μL, 1 eq), EDCI (1.40 g, 7.31 mmol, 2 eq), NMM (1.85 g, 18.27 mmol, 2.01 mL, 5 eq) and Intermediate 1-6 (314.50 mg, 3.65 mmol, 289.07 μL, 1 eq). The mixture was stirred at 25° C. for 1 h. The reaction mixture was quenched by H₂O (20 mL), and extracted with EA (10 mL*3). The combined organic layers were washed with brine (10 mL*3), dried over anhydrous Na₂SO₄, filtered and concentrated under reduced pressure to give a residue. The crude was used for the next step directly without further purification. Intermediate 1-7 (1.1 g, crude) was obtained as yellow oil. LCMS (Method G): Rt=0.569 min, [M+H]⁺=306.2.

### Step 5: Synthesis of Intermediate 1-8

To a solution of Intermediate 1-7 (1.1 g, 3.60 mmol, 1 eq) in THF (4 mL), MeOH (4 mL) and H₂O (4 mL) was added LiOH·H₂O (453.52 mg, 10.81 mmol, 3 eq). The mixture was stirred at 25° C. for 1 h. The mixture was concentrated under vacuum to remove THF and MeOH. Then the aqueous was adjusted to pH=3 by aq. HCl (1N) and extracted with EA (5 ml*3). The combined organic layer was washed with brine (5 ml*3), dried over anhydrous Na₂SO₄, filtered and concentrated under vacuum to give a crude product. The crude was used for the next step directly without purification. Intermediate 1-8 (0.87 g, crude) was obtained as yellow oil. ¹H NMR (400 MHz, DMSO-d₆) δ=13.32-12.74 (m, 1H), 7.77 (dd, J=2.3, 7.1 Hz, 1H), 7.65-7.51 (m, 1H), 7.29 (dd, J=8.6, 10.6 Hz, 1H), 4.62-4.40 (m, 2H), 3.22 (br t, J=11.4 Hz, 1H), 2.96-2.86 (m, 1H), 2.68 (br t, J=11.2 Hz, 1H), 2.08-2.04 (m, 1H), 1.95-1.77 (m, 2H), 1.69-1.42 (m, 2H), 0.85-0.74 (m, 4H).

### Step 6: Synthesis of I-601

To a solution of Intermediate 1-8 (30 mg, 102.98 μmol, 1 eq) in DMF (1 mL) was added HOAt (14.02 mg, 102.98 μmol, 14.41 μL, 1 eq), EDCI (39.48 mg, 205.96 μmol, 2 eq), NMM (52.08 mg, 514.90 μmol, 56.61 μL, 5 eq) and Intermediate 1-9 (75.83 mg, 102.98 μmol, 1 eq, HCl salt). The mixture was stirred at 25° C. for 1 h. The reaction mixture was quenched by H₂O (2 mL), and extracted with EA (1 mL*3). The combined organic layers were washed with brine (1 mL*3), dried over anhydrous Na₂SO₄, filtered and concentrated under reduced pressure to give a residue. The residue was purified by Prep-HPLC (column: Waters Xbridge 150*25 mm*5 um; mobile phase: [water (ammonia hydroxide v/v)-ACN]; gradient: 28%-58% B over 10 min). I-601 (23.99 mg, 24.55 μmol, 23.84% yield, 99.594% purity) was obtained as a yellow solid. LCMS (Method G): Rt=0.661 min, [M+H]⁺=973.6. SFC: Rt=1.900 min. ¹H NMR (400 MHz, METHANOL-d₄) δ=8.36 (br d, J=7.6 Hz, 1H), 7.97-7.91 (m, 1H), 7.90-7.79 (m, 2H), 7.66-7.60 (m, 1H), 7.50-7.35 (m, 3H), 7.16-7.15 (m, 2H), 4.98 (br d, J=6.8 Hz, 1H), 4.65 (br d, J=12.8 Hz, 1H), 4.47 (br d, J=13.2 Hz, 1H), 4.38 (s, 2H), 3.88-3.41 (m, 11H), 3.29-3.18 (m, 3H), 2.91-2.81 (m, 1H), 2.74-2.71 (m, 1H), 2.49 (br d, J=5.2 Hz, 4H), 2.01-1.91 (m, 2H), 1.87-1.42 (m, 18H), 1.39-1.03 (m, 6H), 0.91-0.77 (m, 4H). ¹⁹F NMR (377 MHz, METHANOL-d₄)

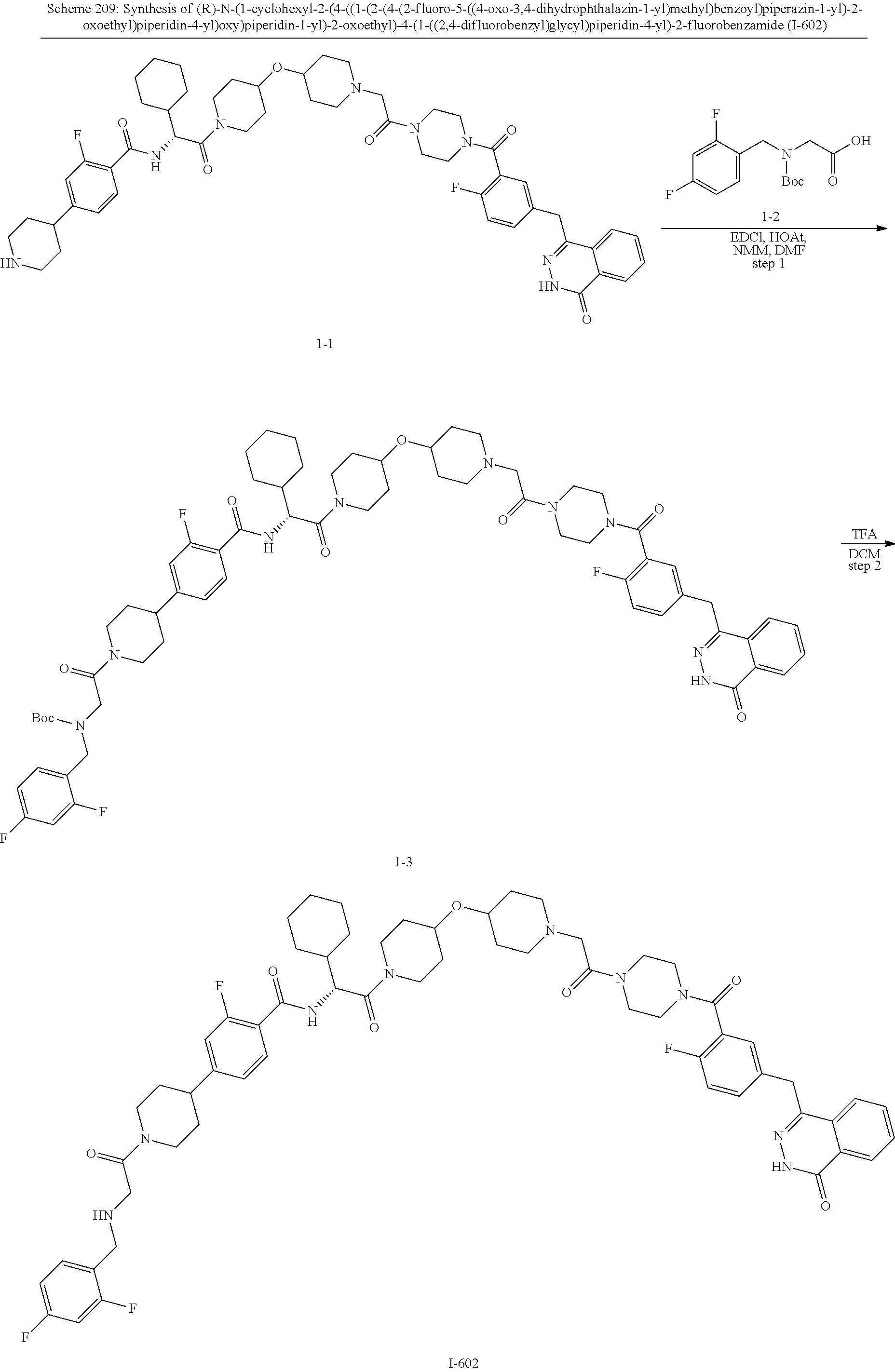

### Step 1: Synthesis of Intermediate 1-3

To a solution of Intermediate 1-1 (80 mg, 82.34 μmol, 1 eq, HCl salt) and Intermediate 1-2 (24.81 mg, 82.34 μmol, 1 eq) in DMF (0.8 mL) was added EDCI (31.57 mg, 164.68 μmol, 2 eq), HOAt (11.21 mg, 82.34 μmol, 11.52 μL, 1 eq) and NMM (41.64 mg, 411.70 μmol, 45.26 μL, 5 eq). The mixture was stirred at 25° C. for 1 hr. To the reaction mixture was added H₂O (2 mL) and then it was extracted with DCM (2 mL*3), the combined organic phase was dried by Na₂SO₄, filtered and concentrated. The crude product was purified by reversed-phase HPLC (0.1% TFA condition), and the eluent was concentrated to remove ACN and lyophilized to give Intermediate 1-3 (90 mg, 71.81 μmol, 87.21% yield, 97.211% purity) as a white solid. LCMS (Method D): Retention time: 0.436 min, [M+H]⁺=1218.4.

### Step 2: Synthesis of I-602

To a solution of Intermediate 1-3 (70 mg, 57.45 μmol, 1 eq) in DCM (1 mL) was added TFA (307.00 mg, 2.69 mmol, 200.00 μL, 46.86 eq). The mixture was stirred at 25° C. for 1 hr. The reaction mixture was concentrated under reduced pressure to give a residue. The residue was dissolved in H₂O (20 ml) and ACN (5 ml), then the mixture was lyophilized to give product. I-602 (68.12 mg, 53.91 μmol, 93.83% yield, 97.522% purity, TFA salt) was obtained as a white solid. LCMS (Method D): Retention time: 0.346 min, [M+H]⁺=1118.4. SFC: Retention time: 1.399 min. ¹H NMR (400 MHz, METHANOL-d₄) δ=8.37 (d, J=7.6 Hz, 1H), 8.12 (t, J=7.6 Hz, 1H), 7.99-7.92 (m, 1H), 7.91-7.79 (m, 2H), 7.72 (t, J=8.0 Hz, 1H), 7.66-7.58 (m, 1H), 7.57-7.47 (m, 1H), 7.37 (s, 1H), 7.23-7.07 (m, 5H), 4.99 (s, 1H), 4.67 (d, J=12.8 Hz, 1H), 4.39 (s, 2H), 4.34-4.29 (m, 3H), 4.23 (d, J=7.6 Hz, 1H), 4.20 (s, 1H), 4.17-4.11 (m, 1H), 4.05-3.97 (m, 1H), 3.95 (s, 1H), 3.92-3.76 (m, 5H), 3.75-3.70 (m, 1H), 3.70-3.63 (m, 1H), 3.63-3.54 (m, 1H), 3.53-3.47 (m, 2H), 3.46-3.42 (m, 1H), 3.36 (s, 4H), 3.27-3.21 (m, 1H), 3.18-3.05 (m, 1H), 3.01-2.91 (m, 1H), 2.89-2.78 (m, 1H), 2.30-2.14 (m, 1H), 2.06 (s, 3H), 2.00-1.91 (m, 3H), 1.90-1.76 (m, 5H), 1.76-1.65 (m, 4H), 1.64-1.51 (m, 2H), 1.38-1.03 (m, 6H). ¹⁹F NMR (400 MHz, METHANOL-d₄) δ=−77.303, −108.357, −112.928, −115.233, −120.716.

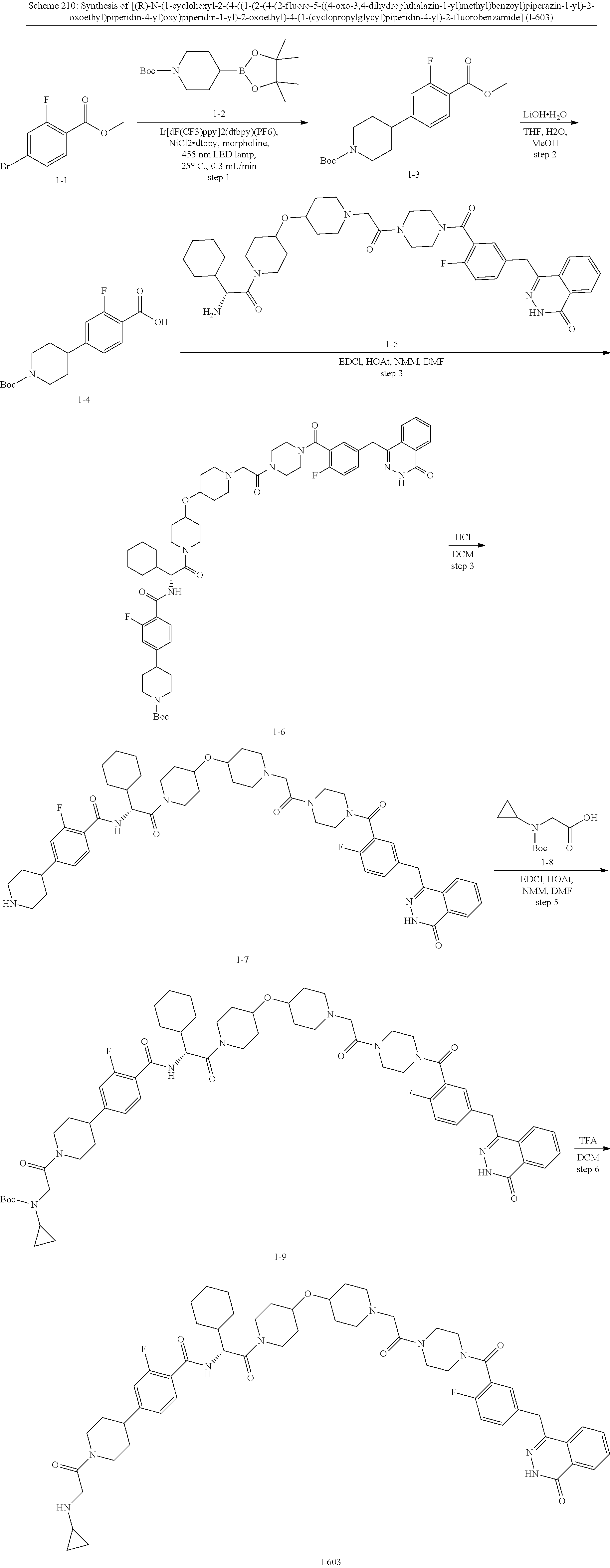

### Step 1: Synthesis of Intermediate 1-3

To a 15 mL vial equipped with a stir bar was added intermediate 1-1 (1 g, 4.29 mmol, 1 eq), intermediate 1-2 (2.67 g, 8.58 mmol, 2 eq), Ir[dF(CF₃)ppy]₂(dtbpy)(PF₆) (48.14 mg, 42.91 μmol, 0.01 eq), NiCl₂-dtbpy (85.39 mg, 214.56 μmol, 0.05 eq), morpholine (560.78 mg, 6.44 mmol, 566.44 μL, 1.5 eq) and DMF (10 mL). The reaction solution was pumped through the reactor at a flow rate of 300 L min, irradiating with a 455 nm LED lamp, with cooling water to keep the reaction temperature at 25° C. for 1 hr. The reaction mixture was diluted with H₂O (15 mL) and extracted with DCM (10 mL*3). The combined organic layers were washed with aq. NaCl (5 mL*2), dried over Na₂SO₄, filtered and concentrated under reduced pressure to give a residue. The residue was purified by flash silica gel chromatography (ISCO; 80 g SepaFlash Silica Flash Column, Eluent of 0˜50% Ethyl acetate/Petroleum ether gradient @60 mL/min) and concentrated under reduced pressure to give intermediate 1-3 (0.86 g, 2.55 mmol, 59.40% yield), as a colorless oil. LCMS (Method D): Retention time: 0.551 min, [M+H]⁺=282.1. ¹H NMR (400 MHz, METHANOL-d4) δ=7.88-7.84 (m, 1H), 7.22-7.02 (m, 2H), 4.22 (d, J=13.2 Hz, 2H), 3.89 (s, 3H), 2.94-2.74 (m, 3H), 1.84 (d, J=12.8 Hz, 2H), 1.65-1.56 (m, 2H), 1.48 (s, 9H).

### Step 2: Synthesis of Intermediate 1-4

To a solution of intermediate 1-3 (60 mg, 177.84 μmol, 1 eq) in THF (0.3 mL), MeOH (0.3 mL), and H₂O (0.3 mL) was added LiOH·H₂O (22.39 mg, 533.52 μmol, 3 eq). The mixture was stirred at 25° C. for 0.5 hr. The reaction mixture was adjusted to pH=6 by HCl (1M), and then extracted with DCM (5 mL*2). The combined organic layers were washed with NaCl (aq) (3 mL*2), dried over Na₂SO₄, filtered and concentrated under reduced pressure to give intermediate 1-4 (60 mg, crude) as a white solid. LCMS (Method D): Retention time: 0.480 min, [M+H]⁺=323.1.

### Step 3: Synthesis of Intermediate 1-6

To a solution of intermediate 1-4 (0.06 g, 185.55 μmol, 1 eq) and intermediate 1-5 (142.20 mg, 185.55 μmol, 1 eq, HCl salt) in DMF (1 mL) was added EDCI (106.71 mg, 556.66 μmol, 3 eq), NMM (93.84 mg, 927.76 μmol, 102.00 μL, 5 eq), and HOAt (25.26 mg, 185.55 μmol, 25.96 μL, 1 eq). The mixture was stirred at 25° C. for 0.5 hr. The reaction mixture was filtered and concentrated under reduced pressure to give a residue. The residue was purified by reversed phase HPLC (0.1% FA condition), and the eluent was concentrated to remove ACN and lyophilized to give intermediate 1-6 (0.11 g, 106.26 μmol, 57.27% yield) as a white solid. LCMS (Method D): Retention time: 0.422 min, [M+H]⁺=1035.4. SFC: Rt: 1.896 min.

### Step 4: Synthesis of Intermediate 1-7

A solution of intermediate 1-6 (0.1 g, 96.60 μmol, 1 eq) in HCl/dioxane (1 mL) was stirred at 25° C. for 0.5 hr. The reaction mixture was concentrated under reduced pressure to give intermediate 1-7 (0.1 g, crude, HCl salt) as a white solid. LCMS (Method D): Retention time: 0.322 min, [M+H]⁺=935.4. SFC: Rt: 1.812 min.

### Step 5: Synthesis of Intermediate 1-9

To a solution of intermediate 1-7 (0.1 g, 102.93 μmol, 1 eq, HCl salt) and intermediate 1-8 (22.15 mg, 102.93 μmol, 1 eq) in DMF (1 mL) was added NMM (52.05 mg, 514.63 μmol, 56.58 μL, 5 eq), HOAt (14.01 mg, 102.93 μmol, 14.40 μL, 1 eq) and EDCI (59.19 mg, 308.78 μmol, 3 eq). The mixture was stirred at 25° C. for 0.5 hr. The reaction mixture was filtered and concentrated under reduced pressure to give a residue. The residue was purified by reversed phase HPLC (0.1% FA condition), and the eluent was concentrated to remove ACN and lyophilized to give intermediate 1-9 (0.07 g, 61.82 μmol, 60.06% yield) as a white solid. LCMS (Method D): Retention time: 0.484 min, [M+H]⁺=1132.7. SFC: Rt: 1.943 min.

### Step 6: Synthesis of I-603

To a solution of intermediate 1-9 (0.06 g, 52.99 μmol, 1 eq) in DCM (0.6 mL) was added TFA (921.00 mg, 8.08 mmol, 600.00 μL, 152.44 eq). The mixture was stirred at 25° C. for 0.5 hr. The reaction mixture was concentrated under reduced pressure to give a residue. The residue was purified by reversed phase HPLC (0.10% TFA condition) and the eluent was concentrated to remove ACN and lyophilized to give I-603 (30.22 mg, 26.21 μmol, 49.47% yield, 99.424% purity, TFA salt) as a white solid. LCMS (Method D): Retention time: 0.325 min, [M+H]⁺=1032.4. SFC: Rt: 2.041 min. ¹H NMR (400 MHz, METHANOL-d4) δ=8.37 (d, J=7.6 Hz, 1H), 8.18-8.02 (m, 1H), 7.99-7.93 (m, 1H), 7.92-7.80 (m, 2H), 7.74-7.70 (m, 1H), 7.51 (d, J=3.6 Hz, 1H), 7.41-7.27 (m, 1H), 7.26-7.06 (m, 3H), 5.04-4.94 (m, 1H), 4.69-4.61 (m, 1H), 4.39 (s, 2H), 4.32-4.11 (m, 4H), 4.06-3.91 (m, 2H), 3.91-3.70 (m, 6H), 3.67-3.60 (m, 1H), 3.57-3.42 (m, 4H), 3.35 (s, 3H), 3.27-3.07 (m, 4H), 3.01-2.91 (m, 1H), 2.89-2.73 (m, 2H), 2.30-2.10 (m, 1H), 2.09-1.48 (m, 18H), 1.33-1.06 (m, 5H), 0.94-0.91 (m, 3H). ¹⁹F NMR (376 MHz, METHANOL-d4) δ=−77.188 (s), −115.246 (s), −120.673-−120.764 (d).

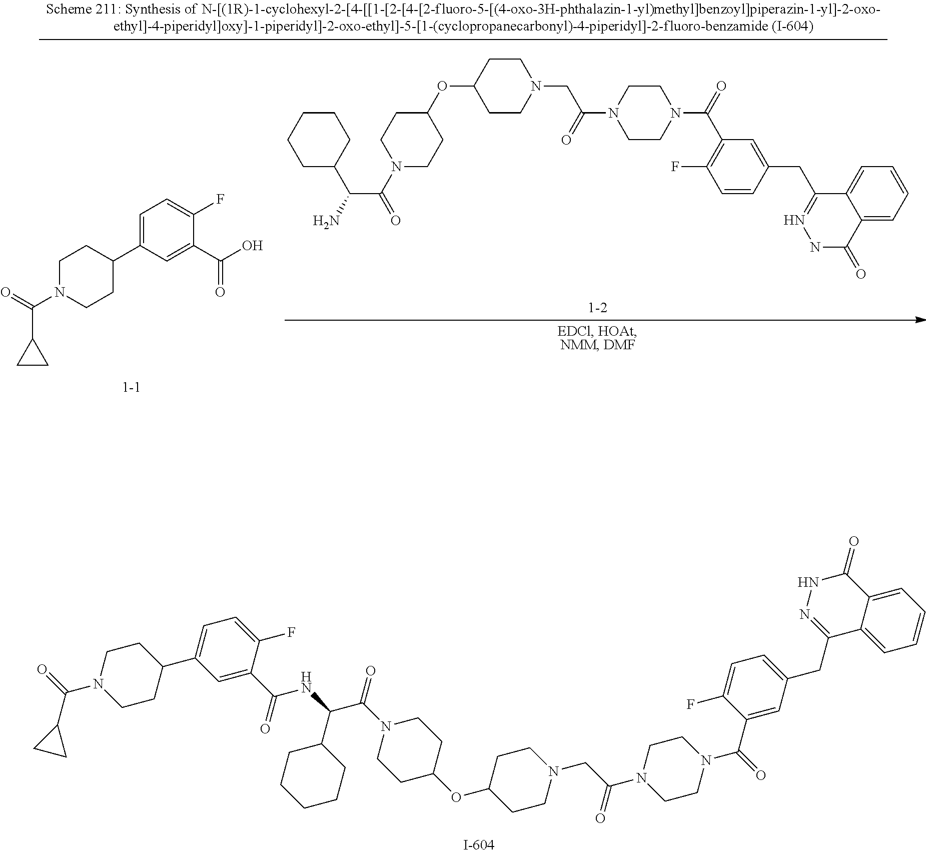

To a solution of Intermediate 1-1 (50 mg, 171.63 μmol, 1 eq) and Intermediate 1-2 (125.27 mg, 163.47 μmol, 9.52e−1 eq, HCl) in DMF (1 mL) was added EDCI (65.81 mg, 343.27 μmol, 2 eq), NMM (86.80 mg, 858.17 μmol, 94.35 μL, 5 eq) and HOAt (23.36 mg, 171.63 μmol, 24.01 μL, 1 eq). The mixture was stirred at 25° C. for 1 hr. The mixture was diluted with water (3 mL), and extracted with EtOAc (5 mL*3). The organic layer was concentrated under vacuum. The residue was purified by Prep-HPLC (column: Waters Xbridge 150*25 mm*5 um; mobile phase: [water (ammonia hydroxide v/v)-ACN]; gradient: 28%-58% B over 10 min) and dried by lyophilization. I-604 (38.64 mg, 38.36 μmol, 22.35% yield, 99.6% purity) was obtained as a white solid. LCMS (Method G): Rt=0.657 min, [M+H]⁺=1003.7. SFC: Rt=5.055 min, ee %=91.04%. ¹H NMR (400 MHz, METHANOL-d₄) δ=8.37 (br d, J=7.6 Hz, 1H), 7.95 (br d, J=7.6 Hz, 1H), 7.90-7.80 (m, 2H), 7.64-7.61 (m, 1H), 7.51-7.36 (m, 3H), 7.19-7.13 (m, 2H), 4.98 (br d, J=7.6 Hz, 1H), 4.66 (br d, J=12.4 Hz, 1H), 4.52-4.43 (m, 1H), 4.38 (s, 2H), 4.09-3.98 (m, 1H), 3.84-3.63 (m, 6H), 3.61-3.43 (m, 5H), 3.29-3.19 (m, 3H), 2.95-2.87 (m, 1H), 2.85-2.70 (m, 3H), 2.41-2.23 (m, 2H), 2.04-1.96 (m, 2H), 1.96-1.76 (m, 10H), 1.73-1.52 (m, 8H), 1.35-1.07 (m, 6H), 0.91-0.80 (m, 4H). ¹⁹F NMR (377 MHz, METHANOL-d₄) δ=−76.921, −119.235, −120.773.

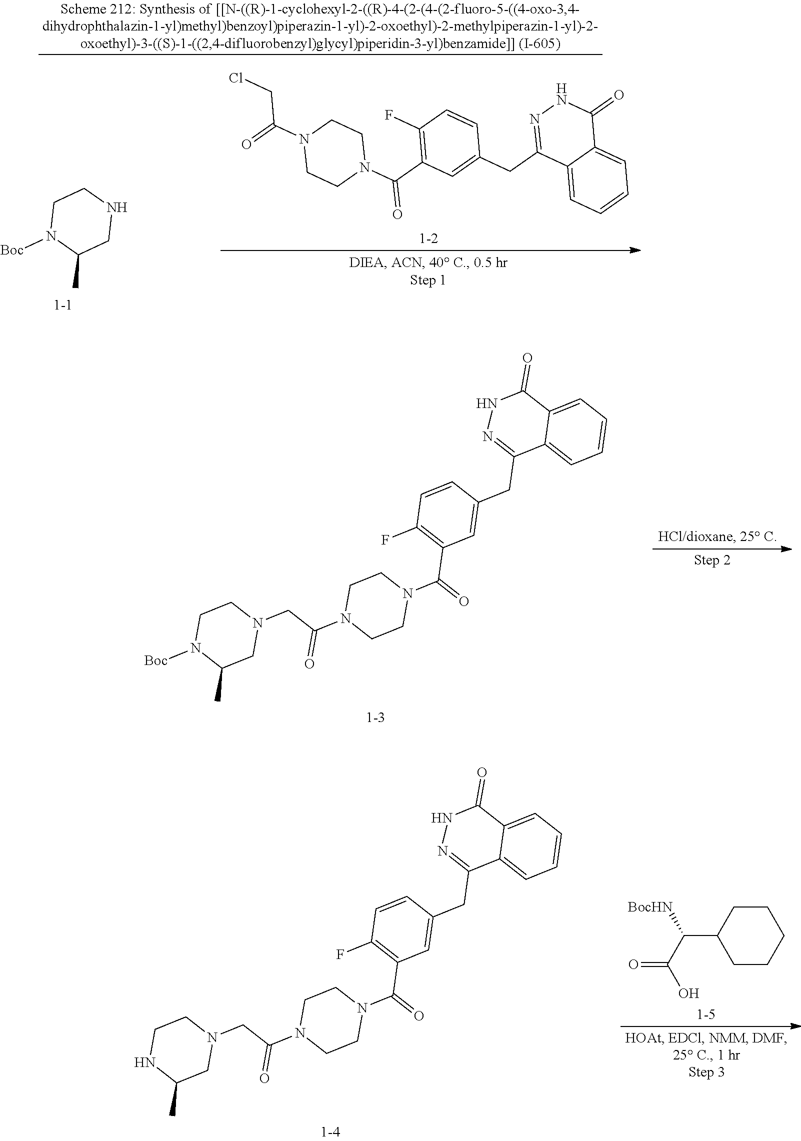 [SMILES: C[C@@H]1CNCCN1C(=O)OC(C)(C)C ; O=C(CCl)N1CCN(C(=O)c2cc(Cc3n[nH]c(=O)c4ccccc34)ccc2F)CC1 ; C[C@@H]1CN(CC(=O)N2CCN(C(=O)c3cc(Cc4n[nH]c(=O)c5ccccc45)ccc3F)CC2)CCN1C(=O)OC(C)(C)C ; C[C@@H]1CN(CC(=O)N2CCN(C(=O)c3cc(Cc4n[nH]c(=O)c5ccccc45)ccc3F)CC2)CCN1 ; CC(C)(C)OC(=O)N[C@@H](C(=O)O)C1CCCCC1]

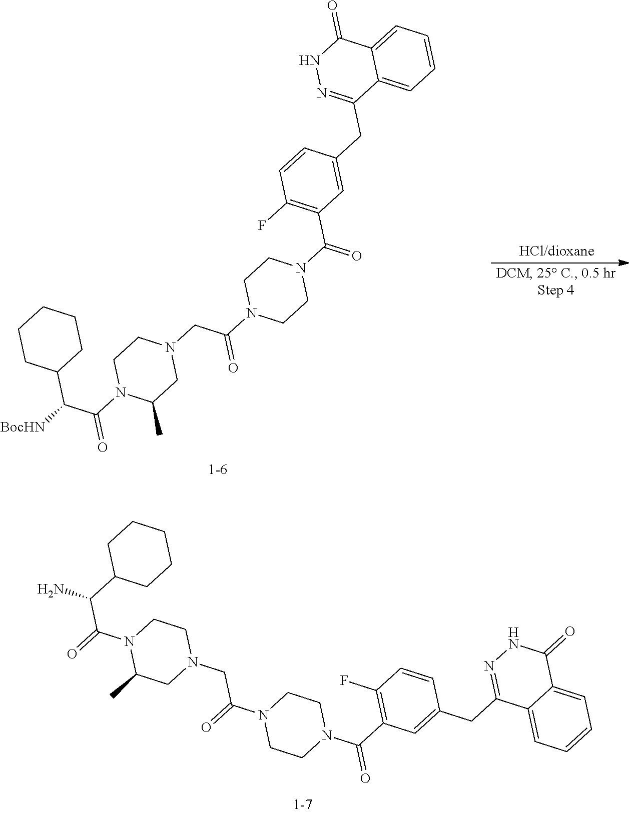 [SMILES: C[C@@H]1CN(CC(=O)N2CCN(C(=O)c3cc(Cc4n[nH]c(=O)c5ccccc45)ccc3F)CC2)CCN1C(=O)[C@H](NC(=O)OC(C)(C)C)C1CCCCC1 ; C[C@@H]1CN(CC(=O)N2CCN(C(=O)c3cc(Cc4n[nH]c(=O)c5ccccc45)ccc3F)CC2)CCN1C(=O)[C@H](N)C1CCCCC1]

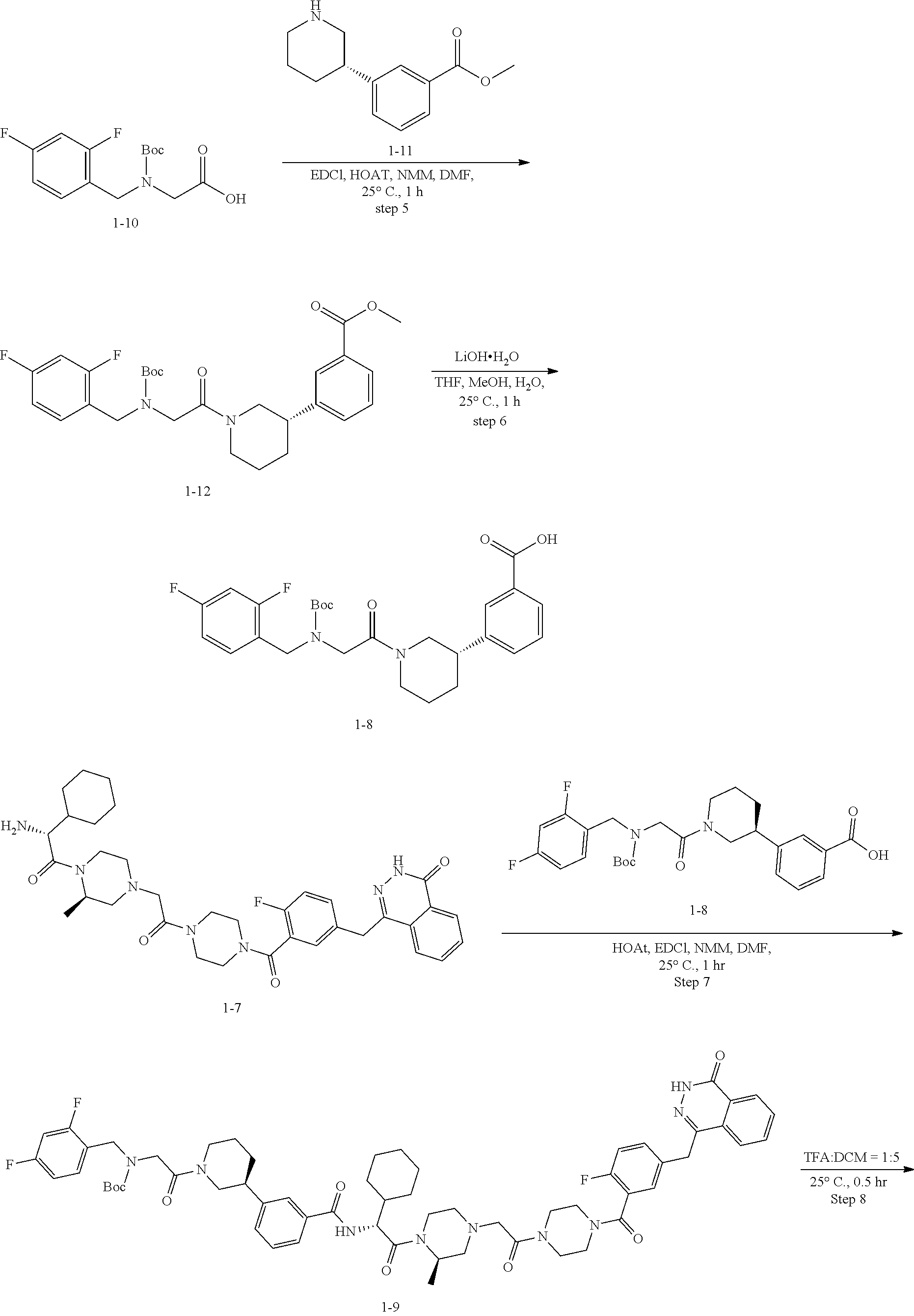

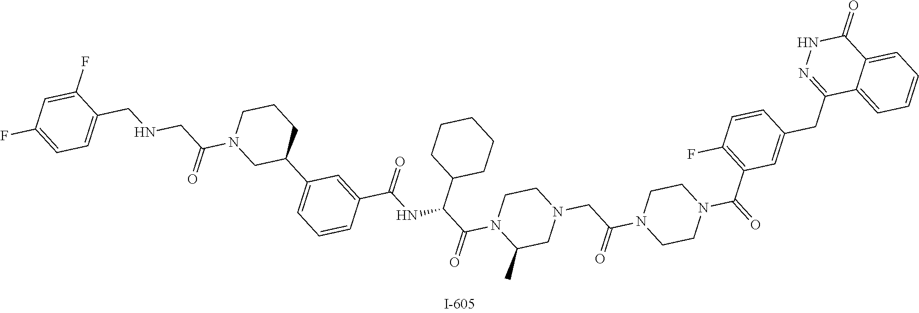 [SMILES: C[C@@H]1CN(CC(=O)N2CCN(C(=O)c3cc(Cc4n[nH]c(=O)c5ccccc45)ccc3F)CC2)CCN1C(=O)[C@H](NC(=O)c1cccc([C@@H]2CCCN(C(=O)CNCc3ccc(F)cc3F)C2)c1)C1CCCCC1]

### Step 1: Synthesis of Intermediate 1-3.

To a solution of intermediate 1-1 (200 mg, 998.62 μmol, 1 eq) and intermediate 1-2 (442.26 mg, 998.62 μmol, 1 eq) in ACN (3 mL) was added DIEA (387.19 mg, 3.00 mmol, 521.82 μL, 3 eq). The mixture was stirred at 40° C. for 0.5 hr. The reaction mixture was concentrated under reduced pressure to give a residue. The residue was purified by flash silica gel chromatography (ISCO; 20 g SepaFlash Silica Flash Column, Eluent of 0˜5% Methanol/Ethyl acetate gradient @60 mL/min), the eluent was concentrated under reduced pressure to give the product. Intermediate 1-3 (350 mg, 573.57 μmol, 57.44% yield, 99.422% purity) was obtained as a white solid. LCMS (Method D): Retention time: 0.345 min, [M+H]⁺=607.2. SFC: Retention time: 1.437 min. ¹H NMR (400 MHz, METHANOL-d4) δ=8.37 (d, J=7.6 Hz, 1H), 7.99-7.92 (m, 1H), 7.90-7.81 (m, 2H), 7.49 (s, 1H), 7.42-7.32 (m, 1H), 7.21-7.10 (m, 1H), 4.39 (s, 2H), 4.27-4.09 (m, 1H), 4.02-3.45 (m, 8H), 3.25-3.05 (m, 3H), 2.87-2.57 (m, 2H), 2.26-1.95 (m, 2H), 1.46 (s, 9H), 1.33-1.12 (m, 4H).

### Step 2: Synthesis of Intermediate 1-4.

To a solution of intermediate 1-3 (300 mg, 494.49 μmol, 1 eq) in DCM (2 mL) was added HCl/dioxane (2 M, 2 mL). The mixture was stirred at 25° C. for 0.5 hr. The reaction mixture was concentrated under reduced pressure to give a crude product. The crude product was used in the next step without further purification. Intermediate 1-4 (300 mg, crude, HCl salt) was obtained as a white solid. LCMS (Method D): Retention time: 0.268 min, [M+H]⁺=507.1. SFC: Retention time: 1.324 min.

### Step 3: Synthesis of Intermediate 1-6.

To a solution of intermediate 1-5 (118.47 mg, 460.38 μmol, 1 eq) and intermediate 1-4 (250 mg, 460.38 μmol, 1 eq, HCl salt) in DMF (3 mL) was added HOAt (62.66 mg, 460.38 μmol, 64.40 μL, 1 eq), EDCI (264.77 mg, 1.38 mmol, 3 eq) and NMM (232.83 mg, 2.30 mmol, 253.08 μL, 5 eq). The mixture was stirred at 25° C. for 0.5 hr. The reaction mixture was diluted with H₂O (5 mL) and extracted with EA (10 mL*3). The combined organic layers were washed with brine (10 mL*3), dried over Na₂SO₄, filtered and concentrated under reduced pressure to give a residue. The residue was purified by flash silica gel chromatography (ISCO; 20 g SepaFlash Silica Flash Column, Eluent of 0˜5% Methanol/Ethyl acetate gradient @60 mL/min), and the eluent was concentrated under reduced pressure to give a residue. Intermediate 1-6 (250 mg, 322.94 μmol, 70.15% yield, 96.349% purity) was obtained as a pale yellow solid. LCMS: Retention time (Method D): 0.374 min, [M+H]⁺=746.4. SFC: Retention time: 4.093 min. ¹H NMR (400 MHz, METHANOL-d4) δ=8.37 (d, J=8.0 Hz, 1H), 7.98-7.93 (m, 1H), 7.91-7.80 (m, 2H), 7.53-7.35 (m, 2H), 7.21-7.12 (m, 1H), 4.66-4.51 (m, 1H), 4.40-4.36 (m, 2H), 4.34-4.18 (m, 1H), 4.02-3.80 (m, 3H), 3.77-3.57 (m, 4H), 3.25-3.12 (m, 2H), 2.28-1.99 (m, 2H), 1.75 (d, J=2.8 Hz, 5H), 1.70-1.56 (m, 4H), 1.44 (d, J=4.0 Hz, 12H), 1.30-1.13 (m, 7H).

### Step 4: Synthesis of Intermediate 1-7.

To a solution of intermediate 1-6 (250 mg, 335.17 μmol, 1 eq) in DCM (2 mL) was added HCl/dioxane (2 M, 2 mL). The mixture was stirred at 25° C. for 0.5 hr. The reaction mixture was concentrated under reduced pressure to give a crude product. The crude product was used in the next step without further purification. Intermediate 1-7 (220 mg, 314.76 μmol, 93.91% yield, 97.607% purity, HCl salt) was obtained as a white solid. LCMS (Method D): Retention time: 0.269 min, [M+H]⁺=646.3. SFC: Retention time: 2.365 min. ¹H NMR (400 MHz, METHANOL-d4) δ=8.37 (d, J=6.8 Hz, 1H), 7.94 (s, 1H), 7.90-7.83 (m, 2H), 7.52 (s, 1H), 7.42-7.35 (m, 1H), 7.21-7.15 (m, 1H), 4.39 (br s, 4H), 3.86-3.78 (m, 3H), 3.72 (d, J=14.4 Hz, 2H), 3.57-3.50 (m, 3H), 3.37 (s, 3H), 1.84 (s, 5H), 1.73 (d, J=13.6 Hz, 3H), 1.59 (d, J=4.4 Hz, 1H), 1.38-1.27 (m, 5H), 1.25-1.12 (m, 5H).

### Step 5: Synthesis of Intermediate 1-12

To a solution of Intermediate 1-10 (2.5 g, 8.30 mmol, 1 eq) in DMF (25 mL) was added HOAt (1.13 g, 8.30 mmol, 1.16 mL, 1 eq), EDCI (4.77 g, 24.89 mmol, 3 eq), NMM (4.20 g, 41.49 mmol, 4.56 mL, 5 eq), then Intermediate 1-11 (2.12 g, 8.30 mmol, 1 eq, HCl salt) was added. The mixture was stirred at 25° C. for 1 hr. The reaction mixture was diluted with water (15 mL) and extracted with EA (10 mL*3). The combined organic layers were dried over Na₂SO₄, filtered and concentrated under reduced pressure to give a residue. The residue was purified by flash silica gel chromatography (ISCO; 80 g SepaFlash Silica Flash Column, Eluent of 0˜100% Petroleum ether/Ethyl acetate @60 mL/min). The eluent was concentrated under reduced pressure to give a residue. Intermediate 1-12 (2.2 g, 4.38 mmol, 52.76% yield) was obtained as a yellow oil. LCMS (Method D): Retention time: 0.494 min, [M+H]⁺=503.2.

### Step 6: Synthesis of Intermediate 1-8.

To a solution of Intermediate 1-12 (2.2 g, 4.38 mmol, 1 eq) in MeOH (7 mL), H₂O (7 mL), and THF (7 mL) was added LiOH·H₂O (367.41 mg, 8.76 mmol, 2 eq). The mixture was stirred at 25° C. for 1 hr. The mixture was adjusted to pH 5 with citric acid and extracted with EA (20 mL*3). The combined organic layers were dried over Na₂SO₄, filtered and concentrated under reduced pressure to give a residue. The residue was used in the next step without further purification. Intermediate 1-8 (2.7 g, crude) was obtained as a colorless oil. LCMS (Method D): Retention time: 0.461 min, [M+Na]⁺=511.2. SFC: Retention time: 1.621 min.

### Step 7: Synthesis of Intermediate 1-9.

To a solution of intermediate 1-7 (150 mg, 219.87 μmol, 1 eq, HCl salt) and intermediate 1-8 (107.41 mg, 219.87 μmol, 1 eq) in DMF (2 mL) was added HOAt (29.93 mg, 219.87 μmol, 30.76 μL, 1 eq), EDCI (126.45 mg, 659.61 μmol, 3 eq) and NMM (111.20 mg, 1.10 mmol, 120.86 μL, 5 eq). The mixture was stirred at 25° C. for 0.5 hr. The reaction mixture was diluted with H₂O (5 mL) and extracted with EA (5 mL*3). The combined organic layers were washed with brine (5 mL*3), dried over Na₂SO₄, filtered and concentrated under reduced pressure to give a residue. The residue was purified by flash silica gel chromatography (ISCO; 20 g SepaFlash Silica Flash Column, Eluent of 0˜12% Methanol/Dichloromethane gradient @60 mL/min), the eluent was concentrated under reduced pressure to give the product. Intermediate 1-9 (150 mg, 114.30 μmol, 51.99% yield, 85.060% purity) was obtained as a yellow solid. LCMS (Method D): Retention time: 0.453 min, [M/2+H]⁺=509.0.

### Step 8: Synthesis of I-605

To a solution of intermediate 1-9 (50 mg, 44.79 μmol, 1 eq) in DCM (0.5 mL) was added TFA (0.1 mL). The mixture was stirred at 25° C. for 0.1 hr. The reaction mixture was concentrated under reduced pressure to give a residue. The residue was purified by reversed phase HPLC (0.1% TFA condition) and the eluent was concentrated and lyophilized to give the desired product. I-605 (14.33 mg, 12.59 μmol, 28.11% yield, 99.320% purity, TFA salt) was obtained as a white solid. LCMS (Method D): Retention time: 0.356 min, [M+H]⁺=1016.2. SFC: Retention time: 2.158 min. ¹H NMR (400 MHz, METHANOL-d4) δ=8.37 (d, J=7.6 Hz, 1H), 8.00-7.93 (m, 1H), 7.92-7.83 (m, 2H), 7.80-7.70 (m, 2H), 7.67-7.59 (m, 1H), 7.54-7.43 (m, 3H), 7.35 (d, J=5.2 Hz, 1H), 7.22-7.06 (m, 3H), 5.19-4.93 (m, 1H), 4.73 (s, 1H), 4.58-4.48 (m, 1H), 4.40 (s, 2H), 4.35-4.27 (m, 3H), 4.26-4.19 (m, 2H), 4.17-4.06 (m, 1H), 3.85-3.68 (m, 5H), 3.64-3.48 (m, 4H), 3.45-3.33 (m, 4H), 3.28-3.17 (m, 2H), 3.14-2.99 (m, 1H), 2.96-2.74 (m, 2H), 2.10-2.02 (m, 1H), 2.00-1.86 (m, 4H), 1.82-1.68 (m, 4H), 1.61 (d, J=6.8 Hz, 2H), 1.41-1.22 (m, 5H), 1.18-1.05 (m, 2H). ¹⁹F NMR (376 MHz, METHANOL-d4) δ=−76.72 (s), −108.19 (s), −112.60 (s), −120.42 (s).

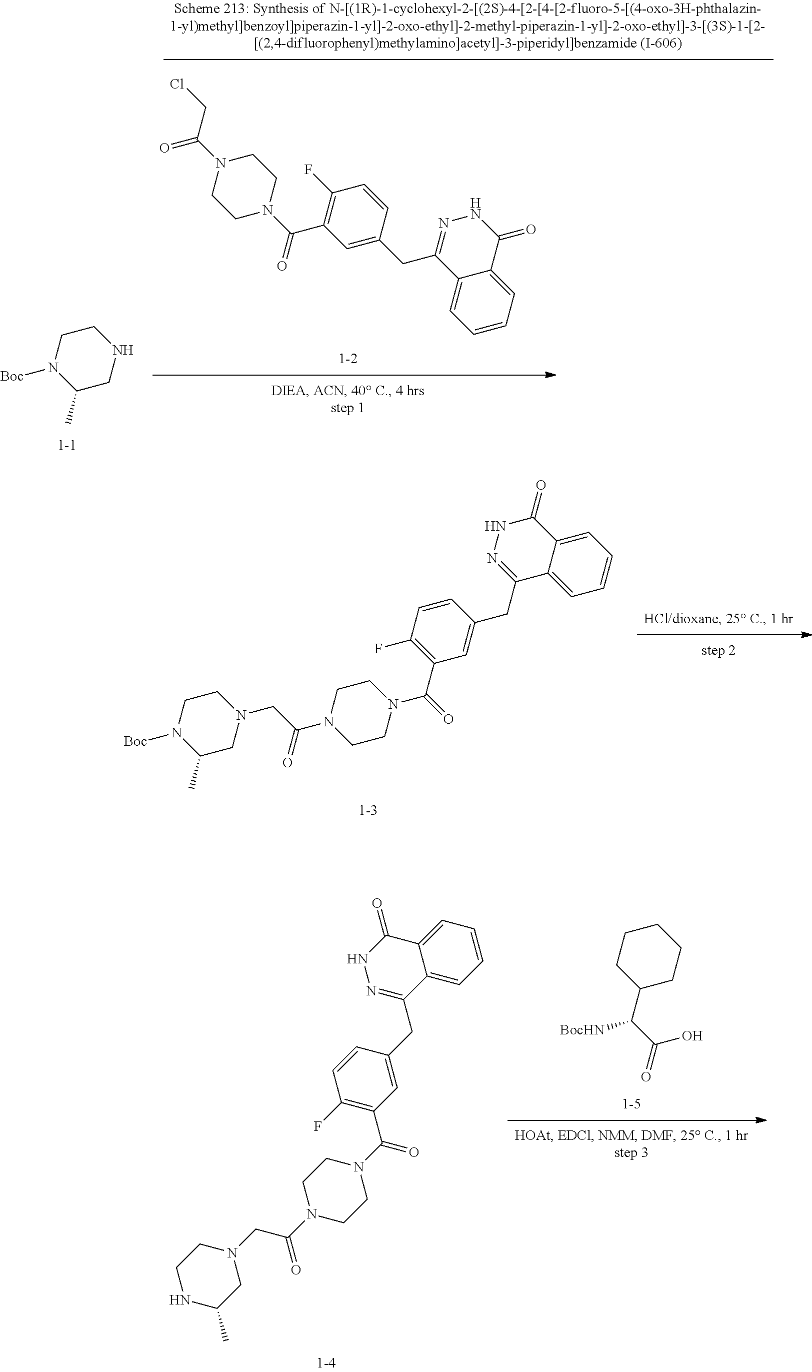 [SMILES: C[C@H]1CNCCN1C(=O)OC(C)(C)C ; O=C(CCl)N1CCN(C(=O)c2cc(Cc3n[nH]c(=O)c4ccccc34)ccc2F)CC1 ; C[C@H]1CN(CC(=O)N2CCN(C(=O)c3cc(Cc4n[nH]c(=O)c5ccccc45)ccc3F)CC2)CCN1C(=O)OC(C)(C)C ; C[C@H]1CN(CC(=O)N2CCN(C(=O)c3cc(Cc4n[nH]c(=O)c5ccccc45)ccc3F)CC2)CCN1 ; CC(C)(C)OC(=O)N[C@@H](C(=O)O)C1CCCCC1]

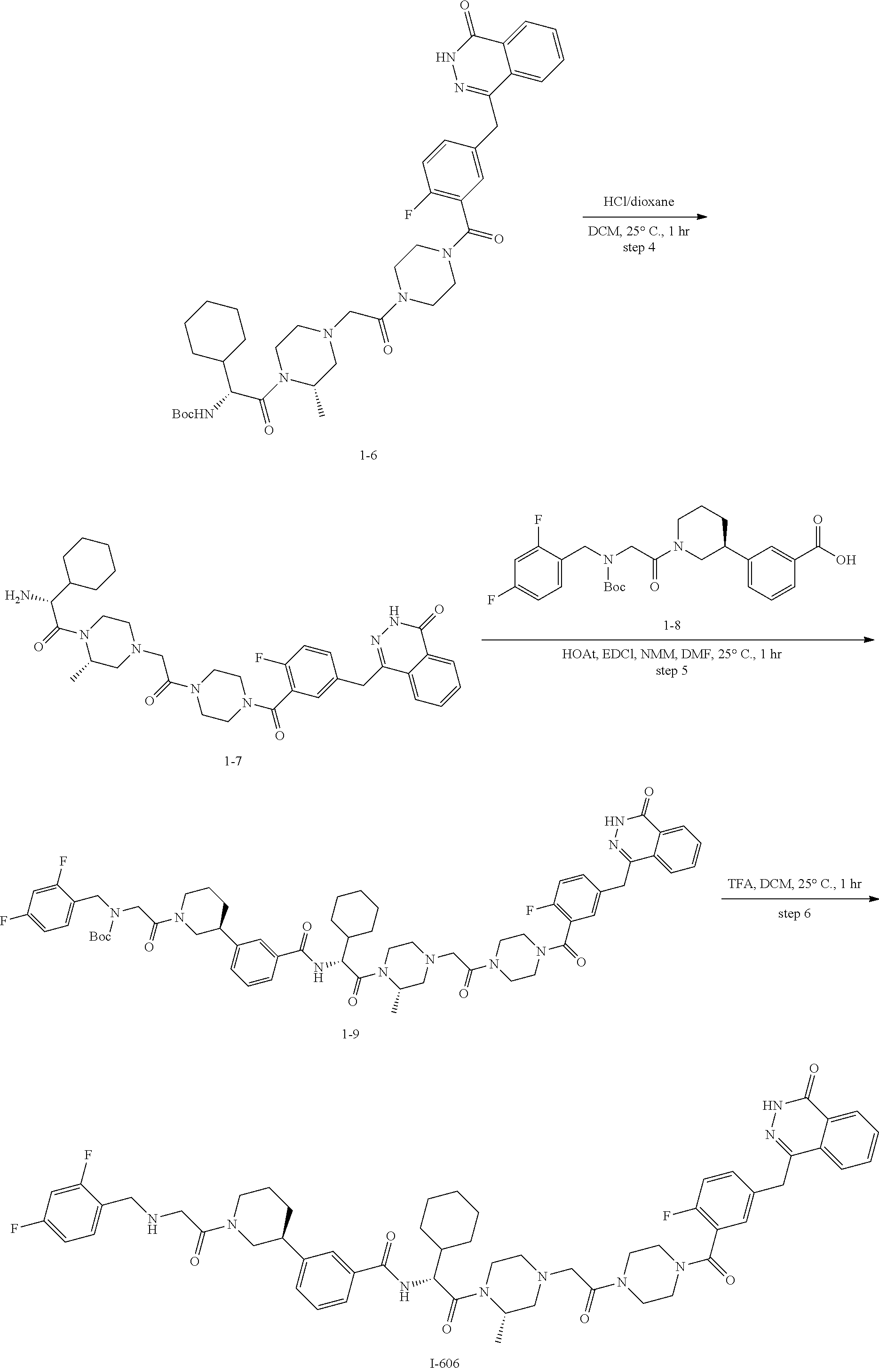

### Step 1: Synthesis of Intermediate 1-3.

To a solution of intermediate 1-1 (162.80 mg, 812.88 μmol, 1.2 eq) in ACN (3 mL) was added intermediate 1-2 (300 mg, 677.40 μmol, 1 eq) and DIEA (350.20 mg, 2.71 mmol, 471.96 μL, 4 eq). The mixture was stirred at 40° C. for 1 h. The reaction mixture was diluted with H₂O (2 mL) and extracted with DCM (3 mL*2). The combined organic layers were dried over Na₂SO₄, filtered and concentrated under reduced pressure to give intermediate 1-3 (400 mg, 622.48 μmol, 91.89% yield, 94.412% purity) as a yellow solid. LCMS (Method D): Retention time: 0.304 min, [M+H]⁺=607.2. SFC: Retention time: 1.363 min.

### Step 2: Synthesis of Intermediate 1-4.

A solution of intermediate 1-3 (350 mg, 576.90 μmol, 1 eq) in DCM (1.4 mL) and HCl/dioxane (2 M, 3.5 mL) was stirred at 25° C. for 1 h. The mixture was concentrated in vacuum to give intermediate 1-4 (400 mg, crude, salt HCl), which was obtained as a yellow solid. LCMS (Method D): Retention time: 0.251 min, [M+H]⁺=507.3. SFC: Retention time: 1.318 min. ¹H NMR (400 MHz, METHANOL-d₄) δ=8.37 (d, J=7.2 Hz, 1H), 7.99-7.93 (m, 1H), 7.92-7.80 (m, 2H), 7.54-7.47 (m, 1H), 7.42-7.34 (m, 1H), 7.22-7.13 (m, 1H), 4.39 (s, 2H), 3.91-3.68 (m, 8H), 3.62-3.54 (m, 3H), 3.48-3.34 (m, 5H), 3.27-3.17 (m, 1H), 1.46-1.43 (m, 3H). ¹⁹FNMR (376 MHz, MeOD-d6) δ=−120.704 (s).

### Step 3: Synthesis of Intermediate 1-6.

To a solution of intermediate 1-5 (165.85 mg, 644.53 μmol, 1 eq) in DMF (3.5 mL) was added intermediate 1-4 (350 mg, 644.53 μmol, 1 eq, HCl salt), EDCI (370.67 mg, 1.93 mmol, 3 eq), HOAt (87.73 mg, 644.53 μmol, 90.16 μL, 1 eq) and NMM (325.96 mg, 3.22 mmol, 354.31 μL, 5 eq). The mixture was stirred at 40° C. for 2 hr. The reaction mixture was diluted with H₂O (3 mL) and extracted with EA (3 mL*2). The combined organic layers were washed with brine (2 mL*1), dried over Na₂SO₄, filtered and concentrated under reduced pressure to give a residue. The residue was purified by prep-HPLC (column: CD01-Phenomenex luna C18 150*25*10 um; mobile phase: [water (FA)-ACN]; gradient: 22%-52% B over 10 min). The eluent was concentrated under reduced pressure to remove ACN and then lyophilized to give intermediate 1-6 (150 mg, 201.10 μmol, 31.20% yield, 100% purity) as a white solid. LCMS (Method D): Retention time: 0.380 min, [M+H]⁺=746.4. SFC: Retention time: 5.918 min.

### Step 4: Synthesis of Intermediate 1-7.

A solution of intermediate 1-6 (130 mg, 174.29 μmol, 1 eq) in DCM (0.5 mL) and HCl/dioxane (2 M, 1.5 mL) was stirred at 25° C. for 1 h. The mixture was concentrated in vacuum to give intermediate 1-7 (130 mg, crude, HCl salt) as yellow oil. LCMS (Method D): Retention time: 0.272 min, [M+H]⁺=646.2. SFC Data: Retention time: 1.024 min. ¹H NMR (400 MHz, METHANOL-d₄) δ=8.39 (d, J=7.6 Hz, 1H), 8.01-7.95 (m, 1H), 7.94-7.83 (m, 2H), 7.57-7.51 (m, 1H), 7.43-7.36 (m, 1H), 7.23-7.16 (m, 1H), 4.48-4.37 (m, 5H), 4.28-4.21 (m, 1H), 3.92-3.71 (m, 5H), 3.64-3.52 (m, 4H), 3.52-3.45 (m, 1H), 3.43-3.36 (m, 3H), 3.29-3.25 (m, 1H), 1.91-1.84 (m, 3H), 1.79-1.67 (m, 3H), 1.59-1.56 (m, 1H), 1.47-1.42 (m, 2H), 1.36-1.30 (m, 4H), 0.95-0.87 (m, 1H). ¹⁹FNMR (376 MHz, MeOD-d6) δ=−120.757 (s).

### Step 5: Synthesis of Intermediate 1-9.

To a solution of intermediate 1-7 (100 mg, 146.58 μmol, 1 eq, HCl salt) in DMF (1 mL) was added intermediate 1-8 (71.61 mg, 146.58 μmol, 1 eq), EDCI (84.30 mg, 439.74 μmol, 3 eq), HOAt (19.95 mg, 146.58 μmol, 20.50 μL, 1 eq) and NMM (74.13 mg, 732.90 μmol, 80.58 μL, 5 eq). The mixture was stirred at 40° C. for 1 h. The reaction mixture was diluted with H₂O (1 mL) and extracted with EA (2 mL*2). The combined organic layers were washed with brine (2 mL*1), dried over Na₂SO₄, filtered and concentrated under reduced pressure to give intermediate 1-9 (160 mg, 143.33 μmol, 97.79% yield), which was obtained as a yellow oil. LCMS (Method D): Retention time: 0.457 min, [M+H]⁺=1116.3.

### Step 6: Synthesis of I-606

A solution of intermediate 1-9 (140 mg, 125.42 μmol, 1 eq) in DCM (1.5 mL) and TFA (0.3 mL) was stirred at 25° C. for 1 h. The mixture was concentrated in vacuum to give a crude product. The crude product was purified by prep-HPLC (column: CD01-Phenomenex luna C18 150*25*10 um; mobile phase: [water (TFA)-ACN]; gradient: 18%-48% B over 10 min). The eluent was concentrated under reduced pressure to remove ACN and then lyophilized to give I-606 (31.02 mg, 27.29 μmol, 21.76% yield, 99.441% purity, TFA salt) as a white solid. LCMS (Method D): Retention time: 0.350 min, [M+H]⁺=1016.2. SFC Data: Retention time: 2.407 min. ¹H NMR (400 MHz, METHANOL-d₄) δ=8.61-8.41 (m, 1H), 8.37 (d, J=8.0 Hz, 1H), 7.98-7.92 (m, 1H), 7.91-7.81 (m, 2H), 7.80-7.68 (m, 2H), 7.66-7.56 (m, 1H), 7.55-7.41 (m, 3H), 7.41-7.32 (m, 1H), 7.22-7.05 (m, 3H), 5.14-4.89 (m, 1H), 4.71-4.49 (m, 2H), 4.39 (s, 2H), 4.35-4.27 (m, 3H), 4.26-4.07 (m, 3H), 3.87-3.66 (m, 5H), 3.62-3.46 (m, 4H), 3.41-3.33 (m, 3H), 3.27-3.03 (m, 3H), 2.97-2.73 (m, 2H), 2.13-2.02 (m, 1H), 2.00-1.54 (m, 11H), 1.46-1.37 (m, 2H), 1.36-1.09 (m, 5H). ¹⁹FNMR (376 MHz, MeOD-d6) δ=−77.127 (s), −108.373 (s), −112.838 (s), −120.652-−120.734 (m).

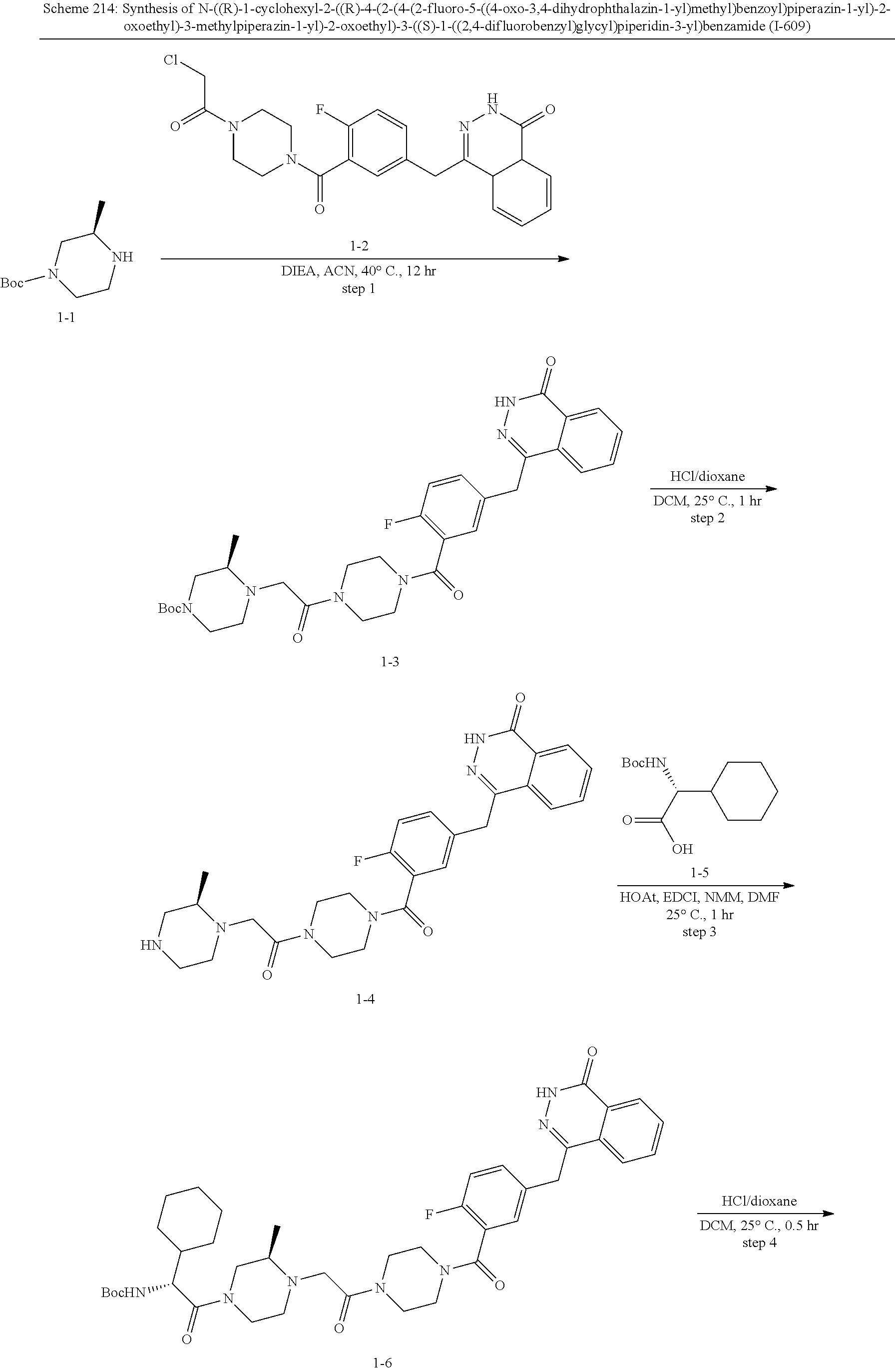 [SMILES: C[C@@H]1CN(C(=O)OC(C)(C)C)CCN1 ; O=C1NN=C(Cc2ccc(F)c(C(=O)N3CCN(C(=O)CCl)CC3)c2)C2C=CC=CC12 ; C[C@@H]1CN(C(=O)OC(C)(C)C)CCN1CC(=O)N1CCN(C(=O)c2cc(Cc3n[nH]c(=O)c4ccccc34)ccc2F)CC1 ; C[C@@H]1CNCCN1CC(=O)N1CCN(C(=O)c2cc(Cc3n[nH]c(=O)c4ccccc34)ccc2F)CC1 ; CC(C)(C)OC(=O)N[C@@H](C(=O)O)C1CCCCC1 ; C[C@@H]1CN(C(=O)[C@H](NC(=O)OC(C)(C)C)C2CCCCC2)CCN1CC(=O)N1CCN(C(=O)c2cc(Cc3n[nH]c(=O)c4ccccc34)ccc2F)CC1]

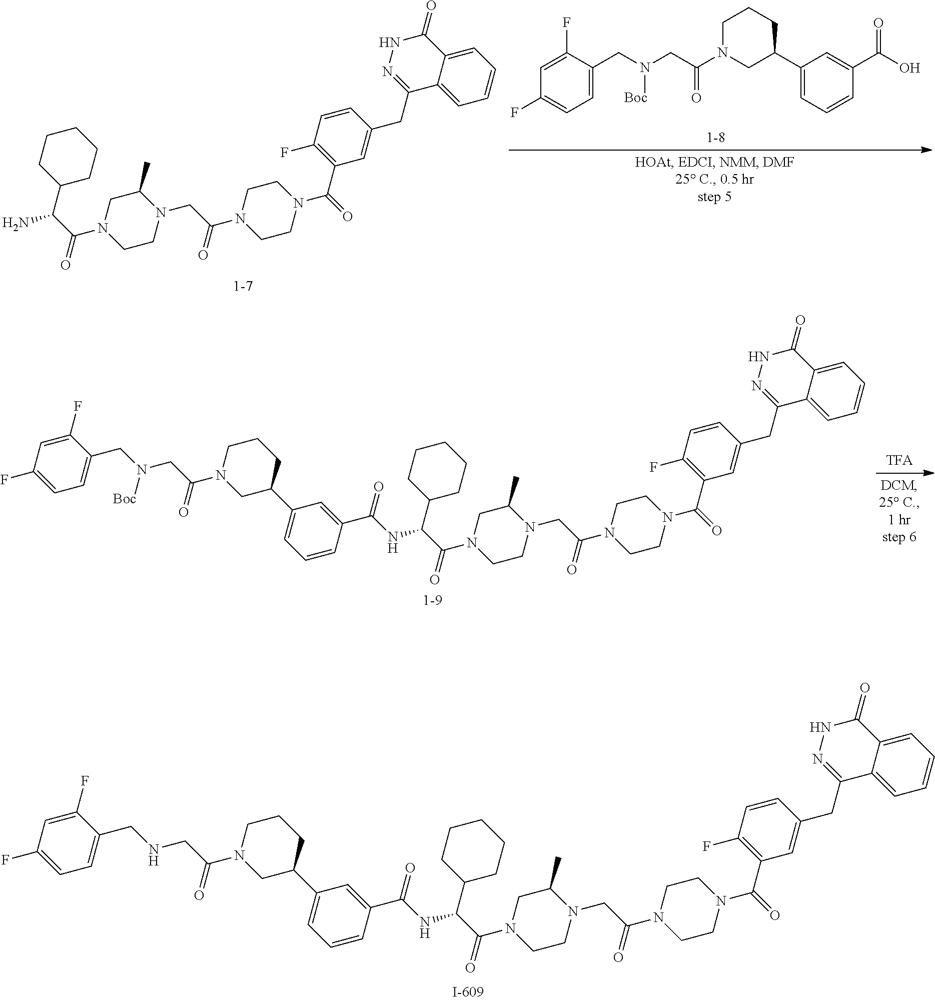

### Step 1: Synthesis of Intermediate 1-3

To a solution of intermediate 1-2 (500 mg, 1.13 mmol, 1 eq) in ACN (5 mL) was added DIEA (437.74 mg, 3.39 mmol, 589.95 μL, 3 eq) and intermediate 1-1 (226.11 mg, 1.13 mmol, 1 eq). The mixture was stirred at 40° C. for 12 h. The mixture was washed with water (10 mL) and extract with EA (5 mL*3). The combined organic layers were dried over sodium sulfate, filtered and concentrated under reduced pressure to give a residue. The residue was purified by flash silica gel chromatography (ISCO; 20 g SepaFlash Silica Flash Column, Eluent of 0˜10% EA/MeOH gradient @50 mL/min) and then the eluent was concentrated in vacuo to give intermediate 1-3 (490 mg, 801.89 μmol, 71.03% yield, 99.285% purity) as yellow solid. LCMS (Method D): Rt=0.318 min, [M+H]⁺=607.2. SFC: Rt=2.069 min, 2.219 min. ¹H NMR (400 MHz, CHLOROFORM-d) δ=10.62-10.23 (m, 1H), 8.46 (s, 1H), 7.80-7.69 (m, 3H), 7.33 (d, J=2.0 Hz, 2H), 7.06 (m, 1H), 4.29 (s, 2H), 3.96-3.79 (m, 2H), 3.76-3.55 (m, 6H), 3.38-3.16 (m, 3H), 3.10-2.82 (m, 2H), 2.77-2.65 (m, 1H), 2.61-2.25 (m, 2H), 1.75-1.60 (m, 1H), 1.46 (s, 9H), 1.07 (d, J=7.8 Hz, 3H). ¹⁹F NMR (377 MHz, CHLOROFORM-d) δ=−117.61 (s).

### Step 2: Synthesis of Intermediate 1-4

To a solution of intermediate 1-3 (440 mg, 725.25 μmol, 1 eq) in DCM (4.5 mL) was added HCl/dioxane (2 M, 4.5 mL). The mixture was stirred at 25° C. for 1 h. The reaction mixture was concentrated to give a crude product. The crude product was used in the next step without further purification. Intermediate 1-4 (550 mg, crude, HCl salt) was obtained as yellow solid. LCMS (Method D): Rt=0.242 min, [M+H]⁺=507.2. SFC: Rt: 1.194 min.

### Step 3: Synthesis of Intermediate 1-6

To a solution of intermediate 1-5 (113.73 mg, 441.96 μmol, 1.2 eq) in DMF (2 mL) was added HOAt (50.13 mg, 368.30 μmol, 51.52 μL, 1 eq), EDCI (211.81 mg, 1.10 mmol, 3 eq), NMM (186.26 mg, 1.84 mmol, 202.46 μL, 5 eq) and intermediate 1-4 (200 mg, 368.30 μmol, 1 eq, HCl salt). The mixture was stirred at 25° C. for 1 h. The mixture was washed with water (3 mL) and extracted with EA (2 mL*3). The combined organic layers were dried over sodium sulfate, filtered and concentrated under reduced pressure to give a residue. The residue was purified by reversed phase HPLC (FA condition), the eluent was concentrated under reduced pressure to remove ACN and lyophilized to give intermediate 1-6 (170 mg, 222.71 μmol, 60.47% yield, 97.714% purity) as a white solid. LCMS (Method D): Rt=0.353 min, [M+H]⁺=746.3. SFC: Rt: 4.939 min, 6.692 min. ¹H NMR (400 MHz, METHANOL-d4) δ=8.40-8.33 (m, 1H), 7.99-7.91 (m, 1H), 7.90-7.80 (m, 2H), 7.55-7.46 (m, 1H), 7.40-7.33 (m, 1H), 7.17 (m, 1H), 4.41-4.32 (m, 3H), 4.01-3.80 (m, 3H), 3.79-3.58 (m, 5H), 3.57-3.43 (m, 2H), 3.36 (d, J=2.0 Hz, 1H), 3.18 (s, 1H), 3.17-2.90 (m, 2H), 2.88-2.76 (m, 1H), 2.67-2.50 (m, 1H), 2.48-2.31 (m, 1H), 1.82-1.71 (m, 3H), 1.68-1.55 (m, 3H), 1.43 (s, 9H), 1.30-1.18 (m, 3H), 1.15-1.01 (m, 5H). ¹⁹F NMR (376 MHz, METHANOL-d4) δ=−120.78 (s).

### Step 4: Synthesis of Intermediate 1-7

To a solution of intermediate 1-6 (150 mg, 201.10 μmol, 1 eq) in DCM (1.5 mL) was added HCl/dioxane (2 M, 1.5 mL). The mixture was stirred at 25° C. for 0.5 h. The reaction mixture was concentrated to give a crude product. The crude product was used in the next step without further purification. Intermediate 1-7 (160 mg, crude, HCl salt) was obtained as a white solid. LCMS (Method D): Rt=0.264 min, [M+H]⁺=646.2. ¹H NMR (400 MHz, METHANOL-d4) δ=8.41-8.32 (m, 1H), 8.00-7.93 (m, 1H), 7.92-7.82 (m, 2H), 7.54-7.47 (m, 1H), 7.38 (m, 1H), 7.18 (m, 1H), 4.69-4.55 (m, 1H), 4.47-4.30 (m, 5H), 4.28-4.12 (m, 1H), 3.86 (d, J=5.2 Hz, 1H), 3.79 (d, J=4.4 Hz, 2H), 3.77-3.69 (m, 2H), 3.63-3.52 (m, 5H), 3.43 (s, 2H), 3.36 (d, J=6.0 Hz, 1H), 1.89-1.76 (m, 4H), 1.72 (d, J=12.0 Hz, 2H), 1.51-1.41 (m, 2H), 1.37-1.27 (m, 3H), 1.25-1.14 (m, 3H). ¹⁹F NMR (377 MHz, METHANOL-d4) δ=−120.75 (s).

### Step 5: Synthesis of Intermediate 1-9

To a solution of intermediate 1-8 (71.61 mg, 146.58 μmol, 1 eq) in DMF (1 mL) was added HOAt (19.95 mg, 146.58 μmol, 20.50 μL, 1 eq), EDCI (84.30 mg, 439.74 μmol, 3 eq), NMM (74.13 mg, 732.90 mol, 80.58 μL, 5 eq) and intermediate 1-7 (100 mg, 146.58 μmol, 1 eq, HCl salt). The mixture was stirred at 25° C. for 0.5 h. The mixture was washed with water (1 mL) and extracted with EA (2 mL*3). The combined organic layers dried over sodium sulfate, filtered and concentrated under reduced pressure to give a residue. The residue was purified by reversed phase HPLC (FA condition), and the eluent was concentrated under reduced pressure to remove ACN and lyophilized to give Intermediate 1-9 (150 mg, 134.38 μmol, 91.67% yield, 100% purity) as a white solid. LCMS (Method D): Rt=0.451 min, [M+H]⁺=1116.6.

### Step 6: Synthesis of I-609

To a solution of intermediate 1-9 (120 mg, 107.50 μmol, 1 eq) in DCM (1.2 mL) was added TFA (0.24 mL). The mixture was stirred at 25° C. for 1 h. The reaction mixture was concentrated to give a residue. The residue was purified by reversed phase HPLC (TFA condition), and the eluent was concentrated under reduced pressure to remove ACN and lyophilized to give I-609 (73.97 mg, 65.45 μmol, 60.88% yield, 100% purity, TFA salt) as a white solid. LCMS (Method D): Rt=0.338 min, [M+H]⁺=1016.4. SFC: Rt: 3.584 min, 4.546 min. ¹H NMR (400 MHz, METHANOL-d4) δ=8.62-8.47 (m, 1H), 8.37 (d, J=7.6 Hz, 1H), 8.00-7.91 (m, 1H), 7.90-7.81 (m, 2H), 7.80-7.70 (m, 2H), 7.61 (m, 1H), 7.55-7.43 (m, 3H), 7.37 (m, 1H), 7.22-7.06 (m, 3H), 4.65-4.42 (m, 3H), 4.39 (s, 2H), 4.32 (d, J=13.2 Hz, 3H), 4.26-4.08 (m, 3H), 3.97-3.87 (m, 1H), 3.86-3.62 (m, 6H), 3.57 (s, 3H), 3.45-3.33 (m, 4H), 3.27-3.12 (m, 2H), 2.95-2.83 (m, 1H), 2.78 (m, 1H), 2.12-2.02 (m, 1H), 1.91 (d, J=13.6 Hz, 4H), 1.79 (d, J=9.2 Hz, 2H), 1.75-1.63 (m, 3H), 1.47 (s, 1H), 1.39 (m, 2H), 1.35-1.22 (m, 3H), 1.19-1.09 (m, 2H). ¹⁹F NMR (376 MHz, METHANOL-d4) δ=−77.15 (s), −101.38 (s), −112.83 (s), −120.74 (s).

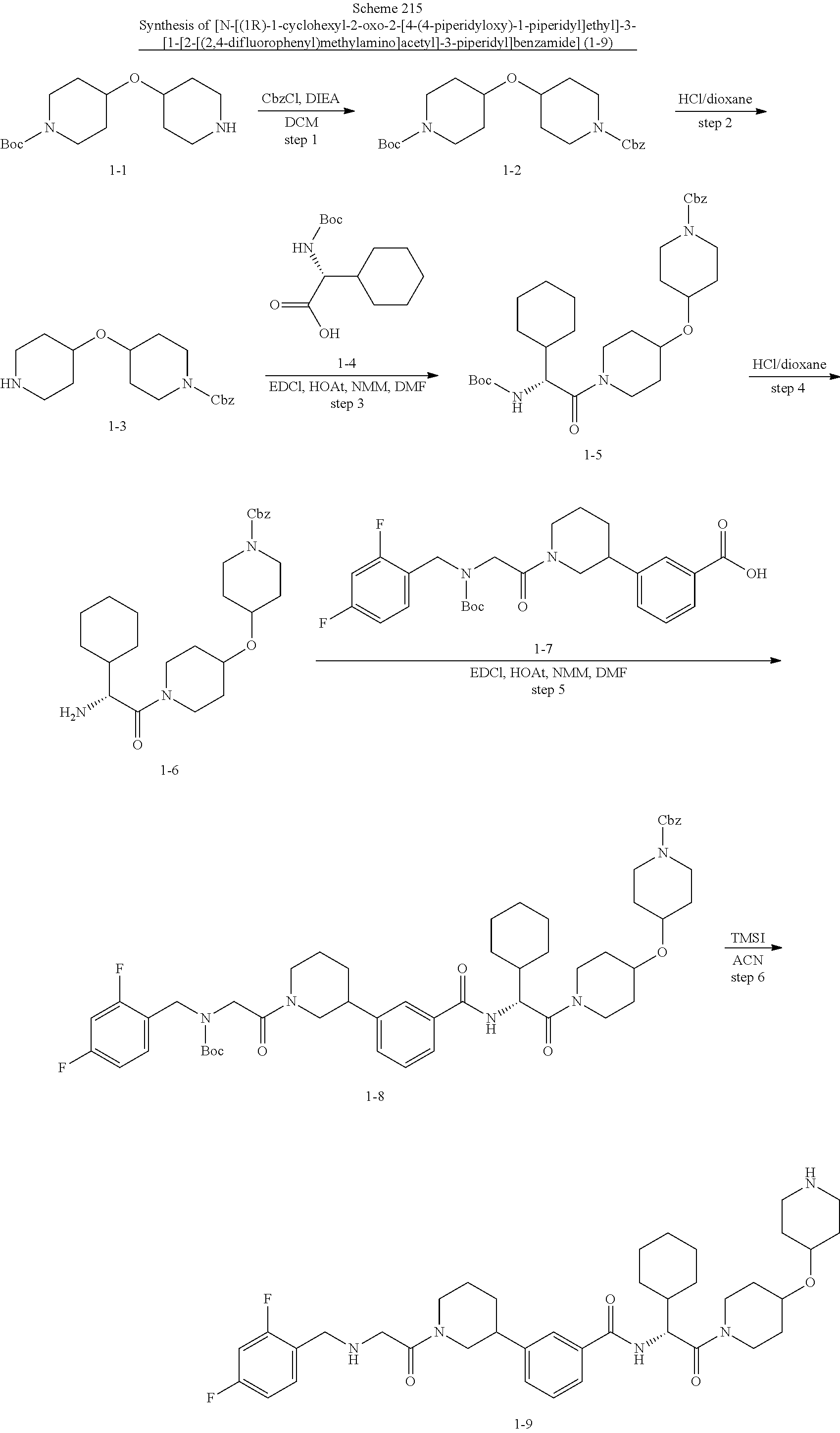

### Step 1: Synthesis of Intermediate 1-2

To a solution of intermediate 1-1 (2 g, 7.03 mmol, 1 eq) and DIEA (1.82 g, 14.07 mmol, 2.45 mL, 2 eq) in DCM (20 mL) was added CbzCl (1.80 g, 10.55 mmol, 1.51 mL, 1.5 eq) at 0° C. The mixture was stirred at 25° C. for 0.5 h. The reaction mixture was washed with H₂O (20 mL) at 25° C., and the mixture was extracted with EA (20 ml*3), and the combined organic phase was dried with anhydrous sodium sulfate, filtered and filtrate was concentrated to give crude product. The crude product was purified by column chromatography (SiO₂, PE/EA=1/0 to 0/1) and the eluent was concentrated under reduced pressure to give intermediate 1-2 (2.9 g, 5.54 mmol, 78.82% yield, 80% purity) as a yellow oil. LCMS (Method D): Retention time=0.572 min, [M+Na]⁺=441.4.

### Step 2: Synthesis of Intermediate 1-3

A solution of intermediate 1-2 (2 g, 4.78 mmol, 1 eq) in HCl/dioxane (2 M, 20.00 mL, 8.37 eq) was stirred at 25° C. for 1 h. The mixture was concentrated under reduced pressure to give intermediate 1-3 (2 g, crude, HCl salt) as a white solid. LCMS (Method D): Retention time=0.248 min, [M+H]⁺=319.1.

### Step 3: Synthesis of Intermediate 1-5

To a solution of intermediate 1-3 (2 g, 5.64 mmol, 1 eq, HCl salt) in DMF (20 mL) was added EDCI (3.24 g, 16.91 mmol, 3 eq), NMM (2.85 g, 28.18 mmol, 3.10 mL, 5 eq), intermediate 1-4 (1.45 g, 5.64 mmol, 1 eq) and HOAt (767.10 mg, 5.64 mmol, 788.39 μL, 1 eq). The mixture was stirred at 25° C. for 0.5 h. The mixture was purified by column chromatography (SiO₂, PE/EA=1/0 to 0/1) and the eluent was concentrated under reduced pressure to give intermediate 1-5 (3 g, 4.57 mmol, 81.13% yield, 85% purity) as a yellow oil. LCMS (Method D): Retention time=0.526 min, [M+H]⁺=558.3.

### Step 4: Synthesis of Intermediate 1-6

A solution of intermediate 1-5 (200 mg, 358.60 μmol, 1 eq) in HCl/dioxane (2 M, 2 mL, 11.15 eq) was stirred at 25° C. for 1 h. The reaction mixture was concentrated under reduced pressure to give intermediate 1-6 (200 mg, crude, HCl salt) as a white solid. LCMS (Method D): Retention time=0.384 min, (M+H)=458.3.

### Step 5: Synthesis of Intermediate 1-8

To a solution of intermediate 1-6 (200 mg, 404.81 μmol, 1 eq, HCl salt) in DMF (2 mL) was added EDCI (232.80 mg, 1.21 mmol, 3 eq), NMM (204.72 mg, 2.02 mmol, 222.53 μL, 5 eq), intermediate 1-7 (197.76 mg, 404.81 μmol, 1 eq) and HOAt (55.10 mg, 404.81 μmol, 56.63 μL, 1 eq). The mixture was stirred at 25° C. for 0.5 h. The mixture was purified by reversed phase HPLC (0.1% FA condition) and the eluent was lyophilized to give intermediate 1-8 (220 mg, 225.19 μmol, 55.63% yield, 95% purity) as a yellow solid. LCMS (Method D): Retention time=0.589 min, [M+H]⁺=928.5. SFC: Retention time=1.109 min, 1.882 min.

### Step 6: Synthesis of Intermediate 1-9

To a solution of intermediate 1-8 (200 mg, 215.49 μmol, 1 eq) in ACN (2 mL) was added TMSI (86.24 mg, 430.98 μmol, 58.66 μL, 2 eq). The mixture was stirred at 25° C. for 1 h. The mixture was purified by prep-HPLC (column: CD01-Phenomenex luna C18 150*25*10 um; mobile phase: [water(FA)-ACN]; gradient: 99%-39% B over 10 min) and the eluent was lyophilized to give Intermediate 1-9 (50.86 mg, 67.66 μmol, 31.40% yield, 98.435% purity, FA salt) as a white solid. LCMS (Method D): Retention time=0.361 min, [M+H]⁺=694.4. SFC: Retention time=3.153 min, 3.800 min. NMR (400 MHz, METHANOL-d4) δ=8.43 (s, 2H), 7.83-7.67 (m, 2H), 7.66-7.38 (m, 3H), 7.15-6.97 (m, 2H), 4.98-4.92 (m, 1H), 4.63-4.42 (m, 1H), 4.17 (d, J=12.8 Hz, 2H), 4.08-3.92 (m, 3H), 3.88-3.48 (m, 5H), 3.39-3.33 (m, 1H), 3.30-3.19 (m, 2H), 3.18-3.09 (m, 2H), 3.04-2.71 (m, 2H), 2.13-1.94 (m, 4H), 1.94-1.74 (m, 9H), 1.73-1.47 (m, 5H), 1.40-1.19 (m, 4H), 1.18-1.05 (m, 2H) ¹⁹F NMR (377 MHz, METHANOL-d₄), δ=−110.030 (s), −113.866-−113.888 (d).

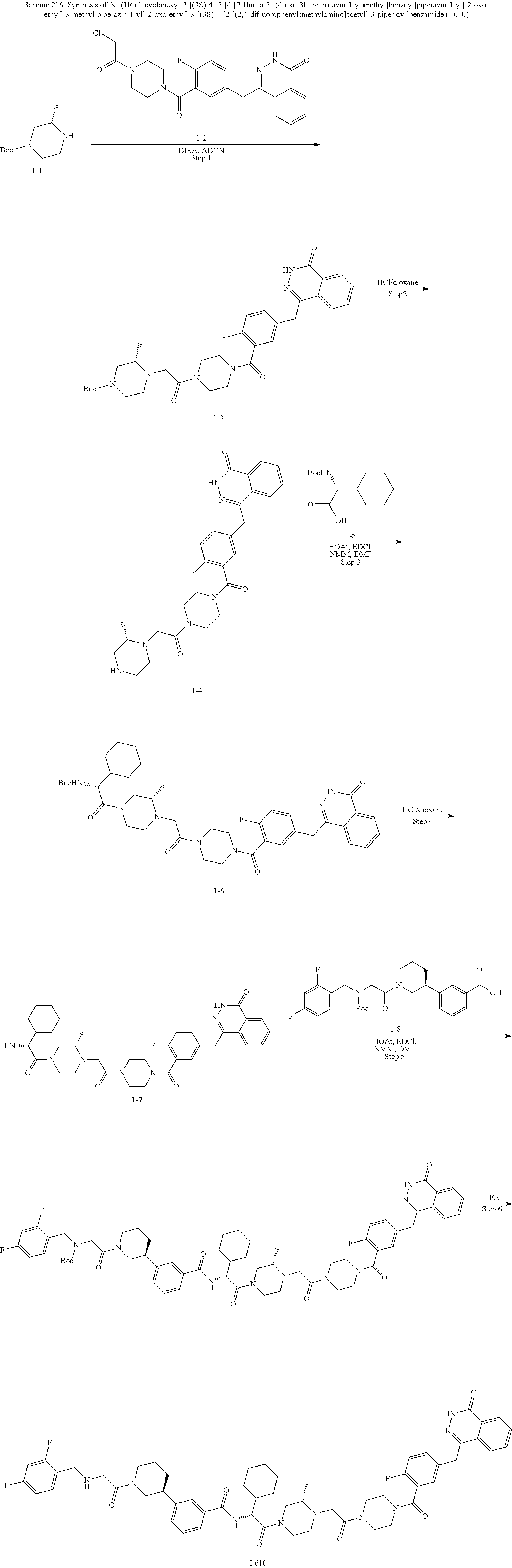

### Step 1: Synthesis of Intermediate 1-3

To a mixture of intermediate 1-1 (542.67 mg, 2.71 mmol, 2 eq) and intermediate 1-2 (600 mg, 1.35 mmol, 1 eq) in ACN (10 mL) was added DIEA (525.29 mg, 4.06 mmol, 707.94 μL, 3 eq), then the mixture was stirred at 40° C. for 1 h. The reaction mixture was diluted with water (80 mL) and extracted with EA (30 mL*2). The combined organic layers were concentrated under reduced pressure to give a residue. The residue was purified by flash silica gel chromatography (ISCO; 20 g SepaFlash Silica Flash Column, Eluent of 2˜10% EA: Methanol@60 mL/min). The eluent was concentrated under reduced pressure to give the product. Intermediate 1-3 (810 mg, 1.34 mmol, 98.55% yield, 100% purity) was obtained as a white solid. LCMS (Method D): Rt: 0.317 min, [M+H]⁺=607.3. SFC: Retention time: 2.070 min.

### Step 2: Synthesis of Intermediate 1-4

To a mixture of intermediate 1-3 (10 g, 37.01 mmol, 1 eq) in DCM (4 mL) was added HCl/dioxane (2 M, 1.57 mL, 5 eq), then the mixture was stirred at 20° C. for 1 h. The reaction mixture was concentrated under reduced pressure to give a residue. Intermediate 1-4 (330 mg, 607.70 μmol, 97.02% yield, HCl salt) was obtained as a white solid. LCMS (Method D): Rt: 0.251 min, [M+H]⁺=607.1. SFC: Retention time: 0.975 min.

### Step 3: Synthesis of Intermediate 1-6

To a solution of intermediate 1-4 (330 mg, 607.70 μmol, 1 eq, HCl salt) and intermediate 1-5 (156.38 mg, 607.70 μmol, 1 eq) in DMF (5 mL) was added HOAt (82.71 mg, 607.70 μmol, 85.01 μL, 1 eq), EDCI (349.49 mg, 1.82 mmol, 3 eq) and NMM (307.33 mg, 3.04 mmol, 334.06 μL, 5 eq), then the mixture was stirred at 40° C. for 1 h. The reaction mixture was diluted with water (80 mL) and extracted with EA (30 mL*2). The combined organic layers were concentrated under reduced pressure to give a residue. Intermediate 1-6 (200 mg, 241.33 μmol, 39.71% yield, 90% purity) was obtained as a colorless oil. LCMS (Method D): Rt: 0.379 min, [M+H]⁺=746.4. SFC: Retention time: 4.763 min.

### Step 4: Synthesis of Intermediate 1-7

To a solution of intermediate 1-6 (180 mg, 241.33 μmol, 1 eq) in DCM (2 mL) was added HCl/dioxane (2 M, 603.31 μL, 5 eq), then the mixture was stirred at 20° C. for 1 h. The reaction mixture was concentrated under reduced pressure to give a residue. Intermediate 1-6 (140 mg, 205.21 mol, 85.03% yield, HCl salt) was obtained as a white solid. LCMS (Method D): Rt: 0.269 min, [M+H]⁺=646.2.

### Step 5: Synthesis of Intermediate 1-9

To a solution of intermediate 1-7 (140 mg, 216.80 μmol, 1 eq) and intermediate 1-8 (105.91 mg, 216.80 μmol, 1 eq) in DMF (3 mL) was added HOAt (29.51 mg, 216.80 μmol, 30.33 μL, 1 eq), EDCI (124.68 mg, 650.39 μmol, 3 eq) and NMM (109.64 mg, 1.08 mmol, 119.18 μL, 5 eq), then the mixture was stirred at 40° C. for 1 h. The reaction mixture was diluted with water (80 mL) and extracted with EA (30 mL*2). The combined organic layers were concentrated under reduced pressure to give a residue. Intermediate 1-9 (240 mg, 215.00 μmol, 99.17% yield) was obtained as a yellow solid. LCMS (Method D): Rt: 0.455 min, [M+H]⁺=1116.2.

### Step 6: Synthesis of I-610

To a mixture of intermediate 1-9 (200 mg, 179.17 μmol, 1 eq) in DCM (2 mL) was added TFA (614.00 mg, 0.4 mL), then the mixture was stirred at 25° C. for 1 h. To the reaction mixture was added NaHCO₃ (about 10 mL) to adjust the pH to 7-8, the mixture was then extracted with DCM (10 mL*3). The combined organic layers were concentrated under reduced pressure to give a residue. The crude product was purified by prep-HPLC (column: CD01-Phenomenex luna C18 150*25*10 um; mobile phase: [water (FA)-ACN]; gradient: 5%-35% B over 8 min). The eluent was concentrated under reduced pressure to remove MeCN and then lyophilized to give the product. I-610 (34 mg, 32.72 μmol, 18.26% yield, 97.805% purity) was obtained as a yellow solid. LCMS (Method D): Rt: 0.354 min, [M+H]⁺=1016.4. SFC: Retention time: 3.982 min. ¹H NMR (400 MHz, METHANOL-d4) δ=8.35 (d, J=7.2 Hz, 1H), 7.98-7.90 (m, 1H), 7.90-7.67 (m, 4H), 7.53-7.34 (m, 5H), 7.18-7.13 (m, 1H), 7.04-6.90 (m, 2H), 4.95-4.88 (m, 2H), 4.59-4.47 (m, 1H), 4.37 (s, 2H), 4.27-4.02 (m, 1H), 3.95-3.87 (m, 3H), 3.81 (d, J=8.4 Hz, 2H), 3.77-3.55 (m, 7H), 3.43-3.34 (m, 1H), 3.27-2.97 (m, 4H), 2.94-2.69 (m, 3H), 2.64-2.24 (m, 2H), 2.02 (s, 1H), 1.97-1.49 (m, 10H), 1.31-1.18 (m, 3H), 1.17-1.01 (m, 5H). ¹⁹F NMR (400 MHz, METHANOL-d4) δ=−112.058-−112.110 (m), −115.300-−115.390 (m), −120.599-−120.689 (m).

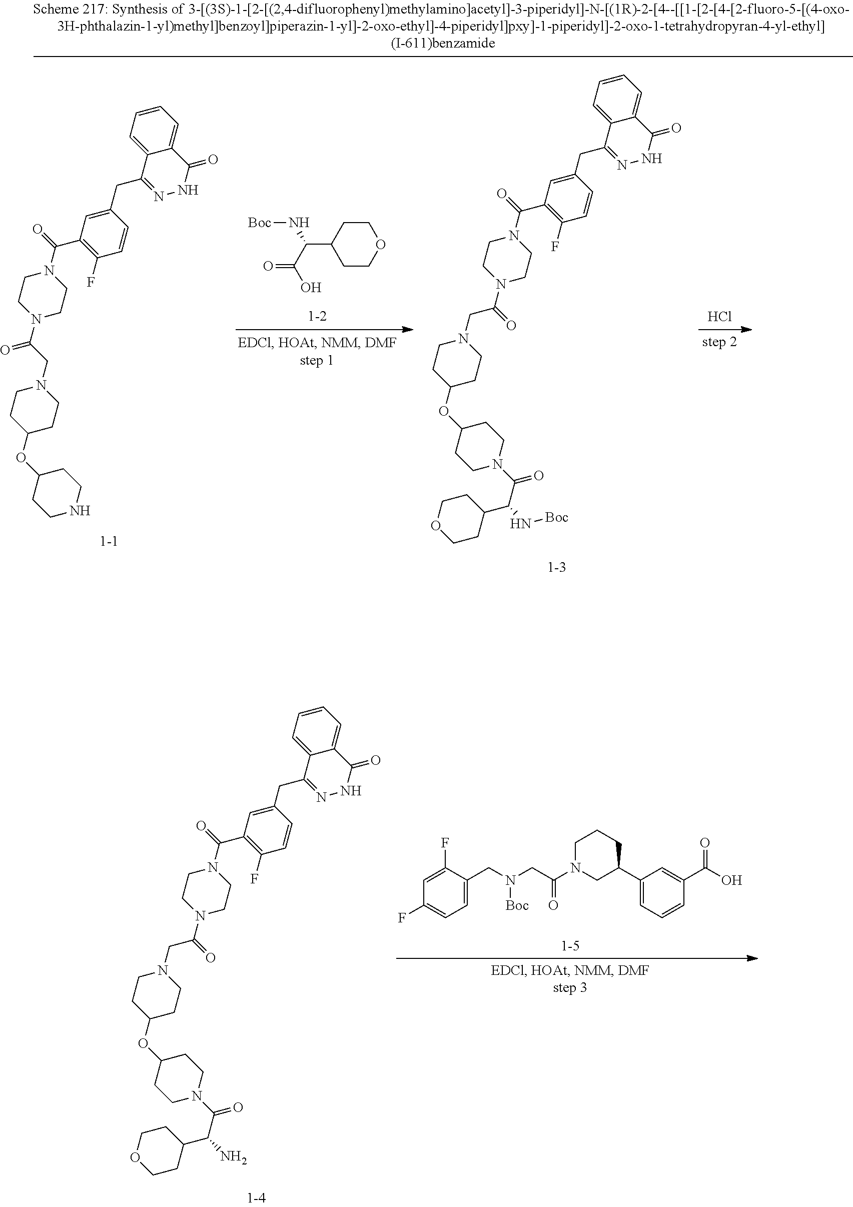 [SMILES: O=C(CN1CCC(OC2CCNCC2)CC1)N1CCN(C(=O)c2cc(Cc3n[nH]c(=O)c4ccccc34)ccc2F)CC1 ; CC(C)(C)OC(=O)N[C@@H](C(=O)O)C1CCOCC1 ; CC(C)(C)OC(=O)N[C@@H](C(=O)N1CCC(OC2CCN(CC(=O)N3CCN(C(=O)c4cc(Cc5n[nH]c(=O)c6ccccc56)ccc4F)CC3)CC2)CC1)C1CCOCC1 ; Cl ; N[C@@H](C(=O)N1CCC(OC2CCN(CC(=O)N3CCN(C(=O)c4cc(Cc5n[nH]c(=O)c6ccccc56)ccc4F)CC3)CC2)CC1)C1CCOCC1 ; CC(C)(C)OC(=O)N(CC(=O)N1CCC[C@@H](c2cccc(C(=O)O)c2)C1)Cc1ccc(F)cc1F]

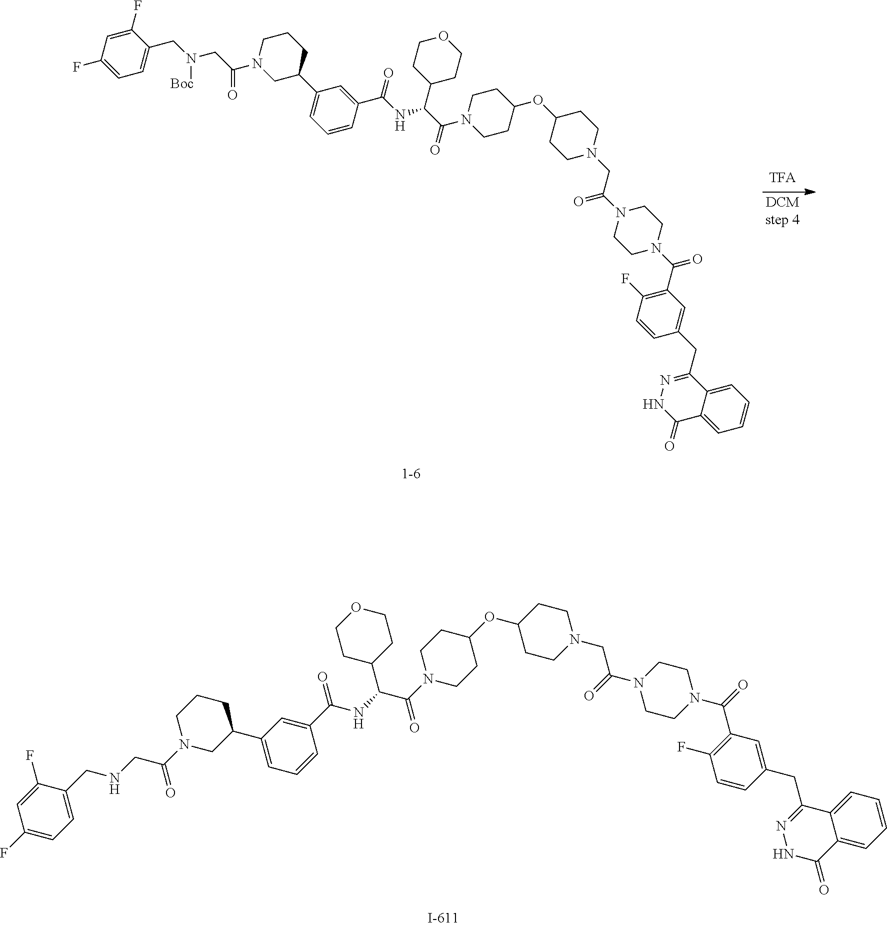 [SMILES: CC(C)(C)OC(=O)N(CC(=O)N1CCC[C@@H](c2cccc(C(=O)N[C@@H](C(=O)N3CCC(OC4CCN(CC(=O)N5CCN(C(=O)c6cc(Cc7n[nH]c(=O)c8ccccc78)ccc6F)CC5)CC4)CC3)C3CCOCC3)c2)C1)Cc1ccc(F)cc1F ; O=C(N[C@@H](C(=O)N1CCC(OC2CCN(CC(=O)N3CCN(C(=O)c4cc(Cc5n[nH]c(=O)c6ccccc56)ccc4F)CC3)CC2)CC1)C1CCOCC1)c1cccc([C@@H]2CCCN(C(=O)CNCc3ccc(F)cc3F)C2)c1]

### Step 1: Synthesis of Intermediate 1-3

To a solution of intermediate 1-2 (250 mg, 964.14 μmol, 1 eq) in DMF (6 mL) was added HOAt (262.46 mg, 1.93 mmol, 269.74 μL, 2 eq), EDCI (924.13 mg, 4.82 mmol, 5 eq), NMM (975.20 mg, 9.64 mmol, 1.06 mL, 10 eq) and intermediate 1-1 (604.66 mg, 964.14 gmol, 1 eq, HCl salt). The mixture was stirred at 25° C. for 0.5 h. The reaction mixture was diluted with water (5 mL) and extracted with EA (5 mL*3). The combined organic layers were dried over Na₂SO₄, filtered and concentrated under reduced pressure to give a residue. The residue was purified by flash silica gel chromatography (ISCO; 40 g SepaFlash Silica Flash Column, Eluent of 0˜100% Petroleum ether/Methanol @60 mL/min). The eluent was concentrated under reduced pressure to give a residue. Intermediate 1-3 (560 mg, 673.10 μmol, 69.81% yield) was obtained as a white solid. LCMS (Method D): Rt=0.338 min [M+H]⁺=832.6. SFC: Retention time: 1.592 min.

### Step 2: Synthesis of Intermediate 1-4

A solution of intermediate 1-3 (500 mg, 600.98 μmol, 1 eq) in HCl/dioxane (2 M, 5.00 mL, 16.64 eq) was stirred at 25° C. for 1.5 h. The reaction mixture was concentrated under reduced pressure to give a residue. The residue was used in the next step without further purification. Intermediate 1-4 (610 mg, crude) was obtained as a yellow oil. LCMS (Method D): Rt=0.239 min [M+H]⁺=732.3. SFC: Retention time: 1.661 min.

### Step 3: Synthesis of Intermediate 1-6

To a solution of intermediate 1-5 (63.58 mg, 130.15 μmol, 1 eq) in DMF (1 mL) was added HOAt (35.43 mg, 260.31 gmol, 36.41 μL, 2 eq), EDCI (124.75 mg, 650.77 μmol, 5 eq), NMM (131.65 mg, 1.30 mmol, 143.10 μL, 10 eq) and intermediate 1-4 (100 mg, 130.15 μmol, 1 eq, HCl salt). The mixture was stirred at 25° C. for 1 h. The reaction mixture was diluted with water (5 mL) and extracted with EA (5 mL*3). The combined organic layers were dried over Na₂SO₄, filtered and concentrated under reduced pressure to give a residue. The residue was used in the next step without further purification. Intermediate 1-6 (90 mg, 74.85 μmol, 57.51% yield) was obtained as a yellow oil. LCMS (Method D): Rt=0.473 min, [M+H]⁺=1202.9.

### Step 4: Synthesis of I-611

To a solution of intermediate 1-6 (80 mg, 66.54 μmol, 1 eq) in DCM (0.6 mL) was added TFA (460.50 mg, 4.04 mmol, 0.3 mL, 60.70 eq). The mixture was stirred at 25° C. for 2 h. The reaction mixture was concentrated under reduced pressure to give a residue. The residue was purified by reversed phase HPLC (0.1% TFA condition). The eluent was lyophilized to give product. I-611 (48 mg, 39.14 μmol, 58.82% yield, 99.173% purity, TFA salt) was obtained as a white solid. LCMS (Method D): Rt=0.332 min, [M+H]⁺=1102.2. ¹H NMR (400 MHz, METHANOL-d4) δ=8.37 (d, J=7.6 Hz, 1H), 7.99-7.93 (m, 1H), 7.91-7.81 (m, 2H), 7.81-7.70 (m, 2H), 7.62 (t, J=6.8 Hz, 1H), 7.55-7.43 (m, 3H), 7.41-7.32 (m, 1H), 7.22-7.06 (m, 3H), 5.02-4.99 (m, 1H), 4.63-4.46 (m, 1H), 4.39 (s, 2H), 4.35-4.28 (m, 3H), 4.27-4.20 (m, 2H), 4.20-4.11 (m, 1H), 4.10-4.02 (m, 1H), 4.01-3.93 (m, 3H), 3.85-3.63 (m, 7H), 3.59-3.43 (m, 5H), 3.43-3.34 (m, 5H), 3.28-3.11 (m, 2H), 3.05-2.74 (m, 2H), 2.25-1.98 (m, 6H), 1.95-1.81 (m, 4H), 1.78-1.29 (m, 8H). ¹⁹F NMR (376 MHz, METHANOL-d₄) δ=−76.985 (s), −108.373 (s), −112.861 (s), −120.667-−120.757 (m). SFC: Retention time: 1.856 min.

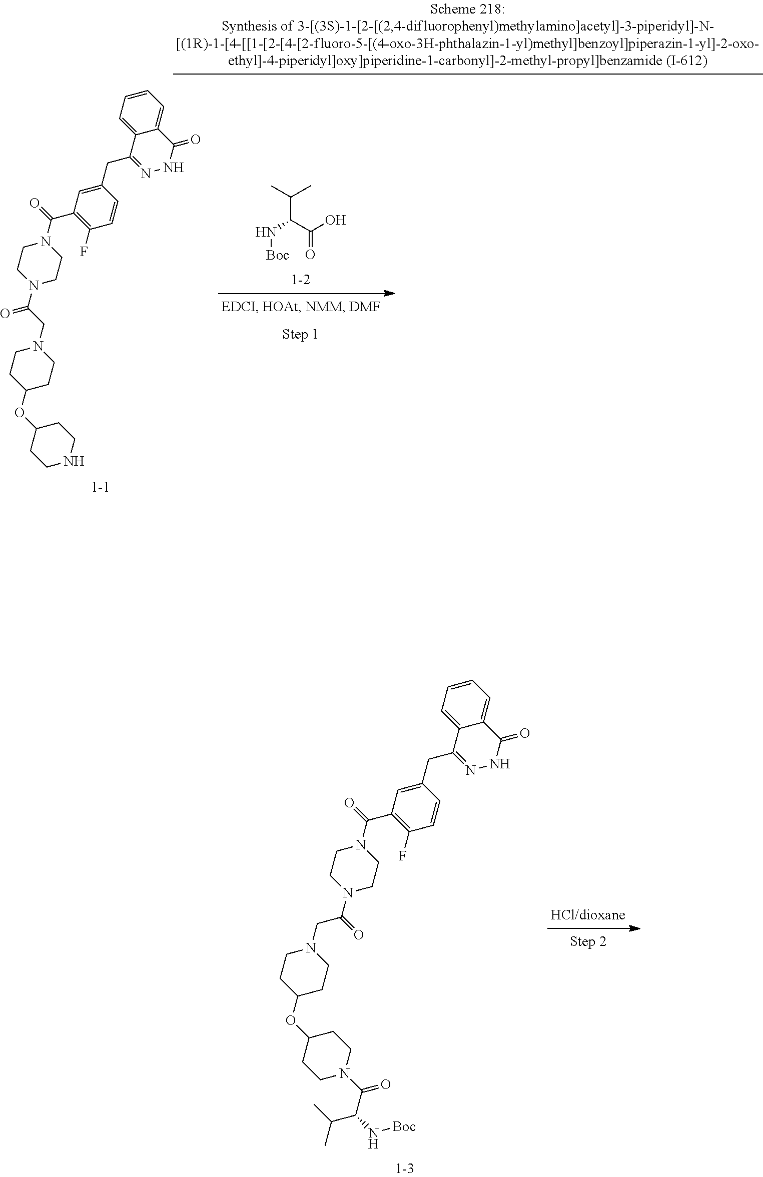 [SMILES: O=C(CN1CCC(OC2CCNCC2)CC1)N1CCN(C(=O)c2cc(Cc3n[nH]c(=O)c4ccccc34)ccc2F)CC1 ; CC(C)[C@@H](NC(=O)OC(C)(C)C)C(=O)O ; CC(C)[C@@H](NC(=O)OC(C)(C)C)C(=O)N1CCC(OC2CCN(CC(=O)N3CCN(C(=O)c4cc(Cc5n[nH]c(=O)c6ccccc56)ccc4F)CC3)CC2)CC1]

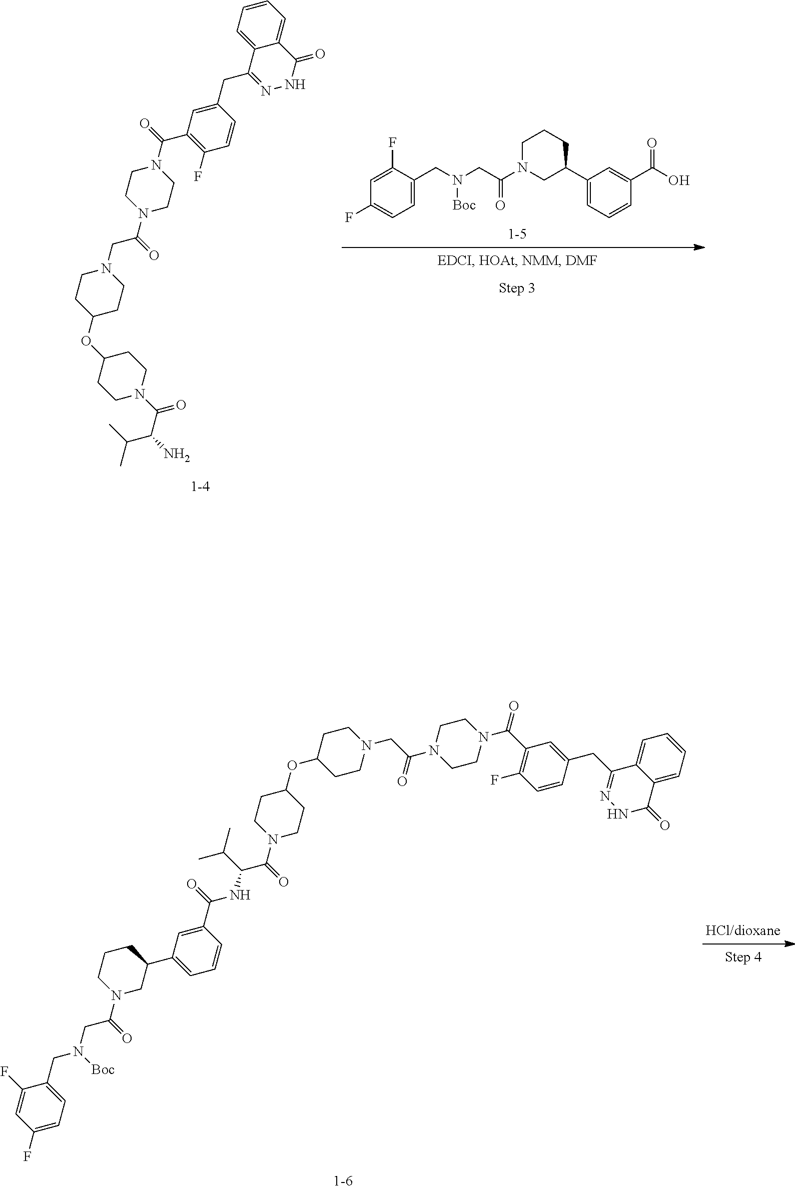 [SMILES: CC(C)[C@@H](N)C(=O)N1CCC(OC2CCN(CC(=O)N3CCN(C(=O)c4cc(Cc5n[nH]c(=O)c6ccccc56)ccc4F)CC3)CC2)CC1 ; CC(C)(C)OC(=O)N(CC(=O)N1CCC[C@@H](c2cccc(C(=O)O)c2)C1)Cc1ccc(F)cc1F ; CC(C)[C@@H](NC(=O)c1cccc([C@@H]2CCCN(C(=O)CN(Cc3ccc(F)cc3F)C(=O)OC(C)(C)C)C2)c1)C(=O)N1CCC(OC2CCN(CC(=O)N3CCN(C(=O)c4cc(Cc5n[nH]c(=O)c6ccccc56)ccc4F)CC3)CC2)CC1]

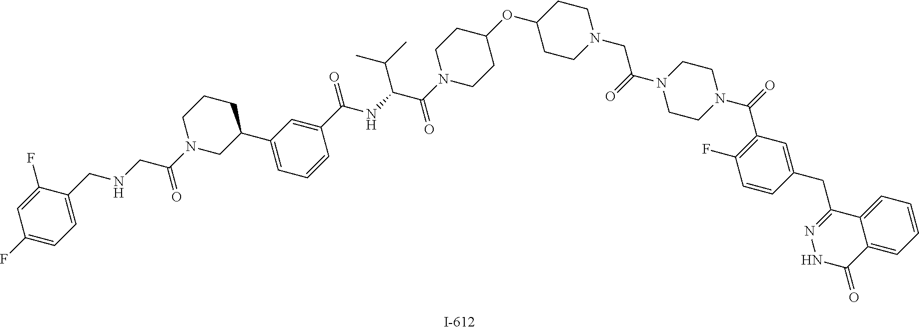 [SMILES: CC(C)[C@@H](NC(=O)c1cccc([C@@H]2CCCN(C(=O)CNCc3ccc(F)cc3F)C2)c1)C(=O)N1CCC(OC2CCN(CC(=O)N3CCN(C(=O)c4cc(Cc5n[nH]c(=O)c6ccccc56)ccc4F)CC3)CC2)CC1]

### Step 1: Synthesis of Intermediate 1-3

To a solution of intermediate 1-2 (69.29 mg, 318.90 μmol, 1 eq) in DMF (2 mL) was added HOAt (86.81 mg, 637.81 μmol, 89.22 μL, 2 eq), EDCI (305.67 mg, 1.59 mmol, 5 eq), NMM (322.57 mg, 3.19 mmol, 350.62 μL, 10 eq) and intermediate 1-1 (200 mg, 318.90 μmol, 1 eq, HCl salt). The mixture was stirred at 25° C. for 1 h. The reaction mixture was diluted with water (5 mL) and extracted with EA (5 mL*3). The combined organic layers were dried over Na₂SO₄, filtered and concentrated under reduced pressure to give a residue. The residue was purified by flash silica gel chromatography (ISCO; 20 g SepaFlash Silica Flash Column, Eluent of 0˜100% Petroleum ether/Ethyl acetate @70 mL/min). The eluent was concentrated under reduced pressure to give a residue. Intermediate 1-3 (210 mg, 265.85 μmol, 83.36% yield) was obtained as a yellow oil. LCMS (Method D): Rt=0.339 min, [M+H]⁺=790.3. SFC: Retention time: 1.399 min.

### Step 2: Synthesis of Intermediate 1-4

A solution of intermediate 1-3 (170 mg, 215.21 μmol, 1 eq) in HCl/dioxane (2 M, 1.70 mL, 15.80 eq) was stirred at 25° C. for 1 h. The reaction mixture was concentrated under reduced pressure to give a residue. The residue was used in the next step without further purification. Intermediate 1-4 (190 mg, crude, HCl salt) was obtained as a yellow oil. LCMS (Method D): Rt=0.260 min, [M+H]⁺=690.5. SFC: Retention time: 1.542 min.

### Step 3: Synthesis of Intermediate 1-6

To a solution of intermediate 1-5 (107.62 mg, 220.30 μmol, 1 eq) in DMF (2 mL) was added HOAt (59.97 mg, 440.60 μmol, 61.63 μL, 2 eq), EDCI (211.16 mg, 1.10 mmol, 5 eq), NMM (222.83 mg, 2.20 mmol, 242.20 μL, 10 eq) and intermediate 1-4 (160 mg, 220.30 μmol, 1 eq, HCl salt). The mixture was stirred at 25° C. for 1 h. The reaction mixture was diluted with water (5 mL) and extracted with EA (5 mL*3). The combined organic layers were dried over Na₂SO₄, filtered and concentrated under reduced pressure to give a residue. The residue was purified by reversed phase HPLC (0.1% FA condition). The eluent was lyophilized to give product. Intermediate 1-6 (190 mg, 163.75 μmol, 74.33% yield) was obtained as a white solid. LCMS (Method D): Rt=0.489 min, [M+H]⁺=1160.7. SFC: Retention time: 1.801 min.

### Step 4: Synthesis of I-612

A solution of intermediate 1-6 (50 mg, 43.09 μmol, 1 eq) in HCl/dioxane (2 M, 0.5 mL, 23.21 eq) was stirred at 25° C. for 20 mins. The reaction mixture was concentrated under reduced pressure to give a residue. The residue was purified by reversed phase HPLC (0.1% NH₃·H₂O condition). The eluent was lyophilized to give product. I-612 (20 mg, 18.24 μmol, 42.32% yield, HCl salt) was obtained as a white solid. LCMS (Method D): Rt=0.336 min, [M+H]⁺=1060.2. ¹H NMR (400 MHz, METHANOL-d4) δ=8.37 (d, J=7.6 Hz, 1H), 7.99-7.93 (m, 1H), 7.91-7.81 (m, 2H), 7.79 (s, 1H), 7.76-7.71 (m, 1H), 7.68-7.58 (m, 1H), 7.56-7.42 (m, 3H), 7.41-7.33 (m, 1H), 7.21-7.07 (m, 3H), 4.95-4.89 (m, 1H), 4.62-4.44 (m, 1H), 4.39 (s, 2H), 4.32 (d, J=11.6 Hz, 3H), 4.28-4.24 (m, 1H), 4.20 (d, J=9.2 Hz, 1H), 4.16-3.91 (m, 3H), 3.85-3.63 (m, 7H), 3.60-3.43 (m, 5H), 3.39-3.33 (m, 3H), 3.27-3.13 (m, 2H), 3.12-2.91 (m, 1H), 2.90-2.76 (m, 1H), 2.28-2.15 (m, 2H), 2.06 (s, 4H), 1.95-1.80 (m, 4H), 1.73-1.48 (m, 3H), 1.37-1.27 (m, 1H), 1.06-0.97 (m, 6H). ¹⁹F NMR (376 MHz, METHANOL-d₄) δ=−108.380 (s), −112.823-−112.876 (m), −120.674-−120.757 (m). SFC: Retention time: 0.731 min.

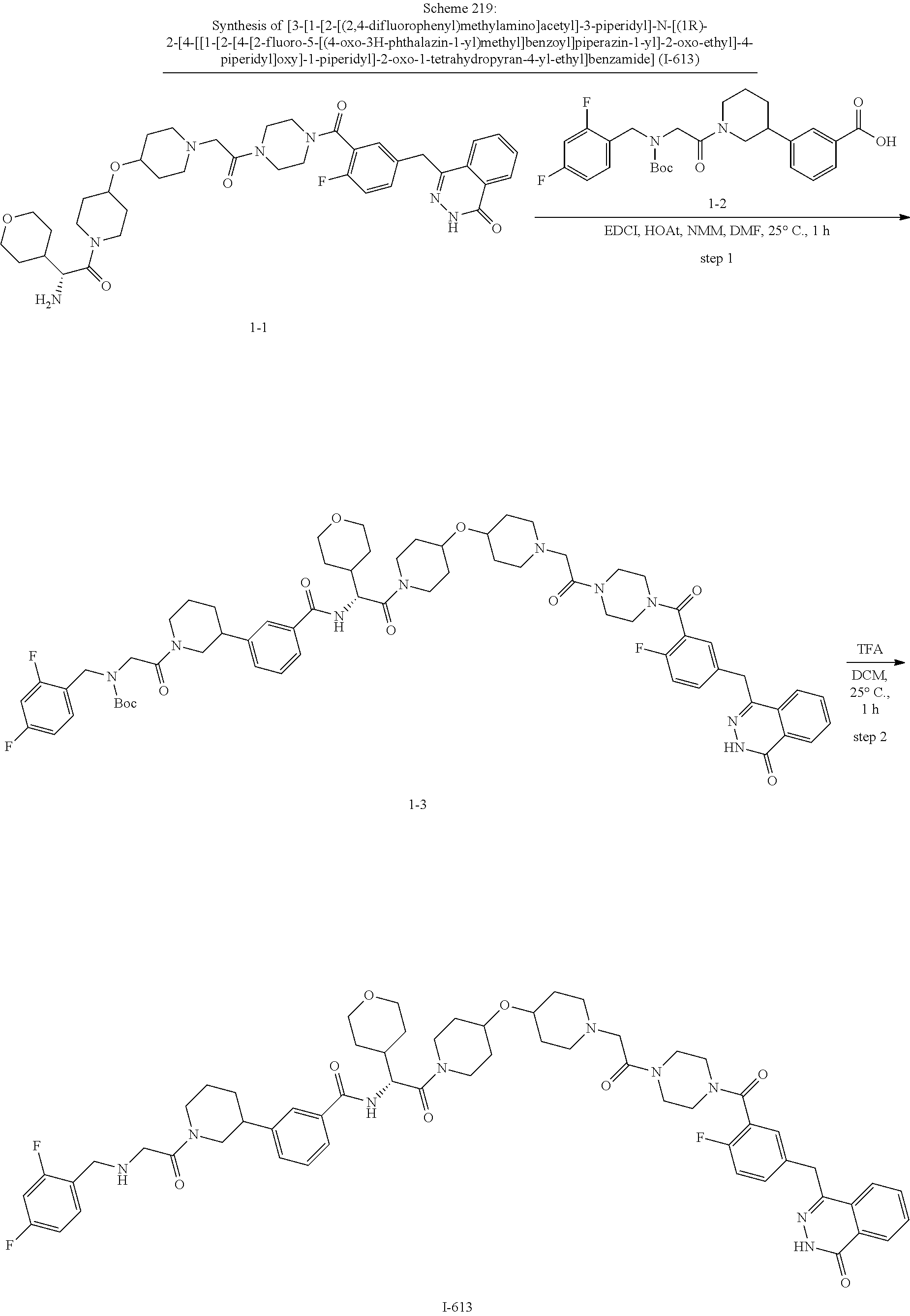

### Step 1: Synthesis of Intermediate 1-3

To a solution of intermediate 1-2 (50 mg, 102.35 μmol, 1 eq) in DMF (0.5 mL) was added EDCI (98.10 mg, 511.75 μmol, 5 eq), NMM (103.52 mg, 1.02 mmol, 112.53 μL, 10 eq) and HOAt (27.86 mg, 204.70 μmol, 28.63 μL, 2 eq), then intermediate 1-1 (95.23 mg, 112.58 μmol, 1.1 eq, TFA salt) was added into the mixture. The mixture was stirred at 25° C. for 2 hrs. The residue was diluted with H₂O (5 mL) and extracted with EA (5 mL*3) dried over Na₂SO₄, filtered and concentrated under reduced pressure to give a residue. Intermediate 1-3 (120 mg, 99.80 μmol, 97.51% yield) was obtained as a yellow oil and was used in the next step without further purification. LCMS (Method D): Rt=0.479 min, [M+H]⁺=1202.6.

### Step 2: Synthesis of I-613

To a solution of intermediate 1-3 (120 mg, 99.80 μmol, 1 eq) in DCM (0.8 mL) was added TFA (0.4 mL). The mixture was stirred at 25° C. for 1 hr. The residue was diluted with H₂O (5 mL) and extracted with EA (5 mL*3) dried over Na₂SO₄, filtered and concentrated under reduced pressure to give a residue. The crude product was purified by reversed-phase HPLC (0.1% TFA condition). The eluent was concentrated under reduced pressure to remove ACN, then lyophilized to afford product. I-613 (22 mg, 18.09 μmol, 18.12% yield, 100% purity, TFA salt) was obtained as a white solid. LCMS (Method D): Rt=0.327 min, [M+H]⁺=1102.2. ¹H NMR (400 MHz, METHANOL-d4) δ=8.37 (d, J=7.2 Hz, 1H), 7.94 (d, J=7.2 Hz, 1H), 7.91-7.81 (m, 2H), 7.80-7.71 (m, 2H), 7.64-7.56 (m, 1H), 7.55-7.42 (m, 3H), 7.40-7.31 (m, 1H), 7.22-7.03 (m, 3H), 5.06-4.94 (m, 1H), 4.65-4.44 (m, 1H), 4.39 (s, 2H), 4.31 (d, J=12.4 Hz, 3H), 4.27-4.04 (m, 4H), 4.03-3.88 (m, 4H), 3.85-3.62 (m, 7H), 3.60-3.46 (m, 4H), 3.46-3.35 (m, 5H), 3.21-3.06 (m, 2H), 3.02-2.74 (m, 2H), 2.27-2.10 (m, 2H), 2.06 (s, 3H), 1.98-1.80 (m, 5H), 1.78-1.62 (m, 3H), 1.61-1.53 (m, 2H), 1.52-1.30 (m, 3H). ¹⁹F NMR: (377 MHz, METHANOL-d₄) δ=−120.673-−120.763 (m), −112.852 (s), −108.357 (s), −77.106 (s). SFC: Retention time: 4.648 min, 5.358 min.

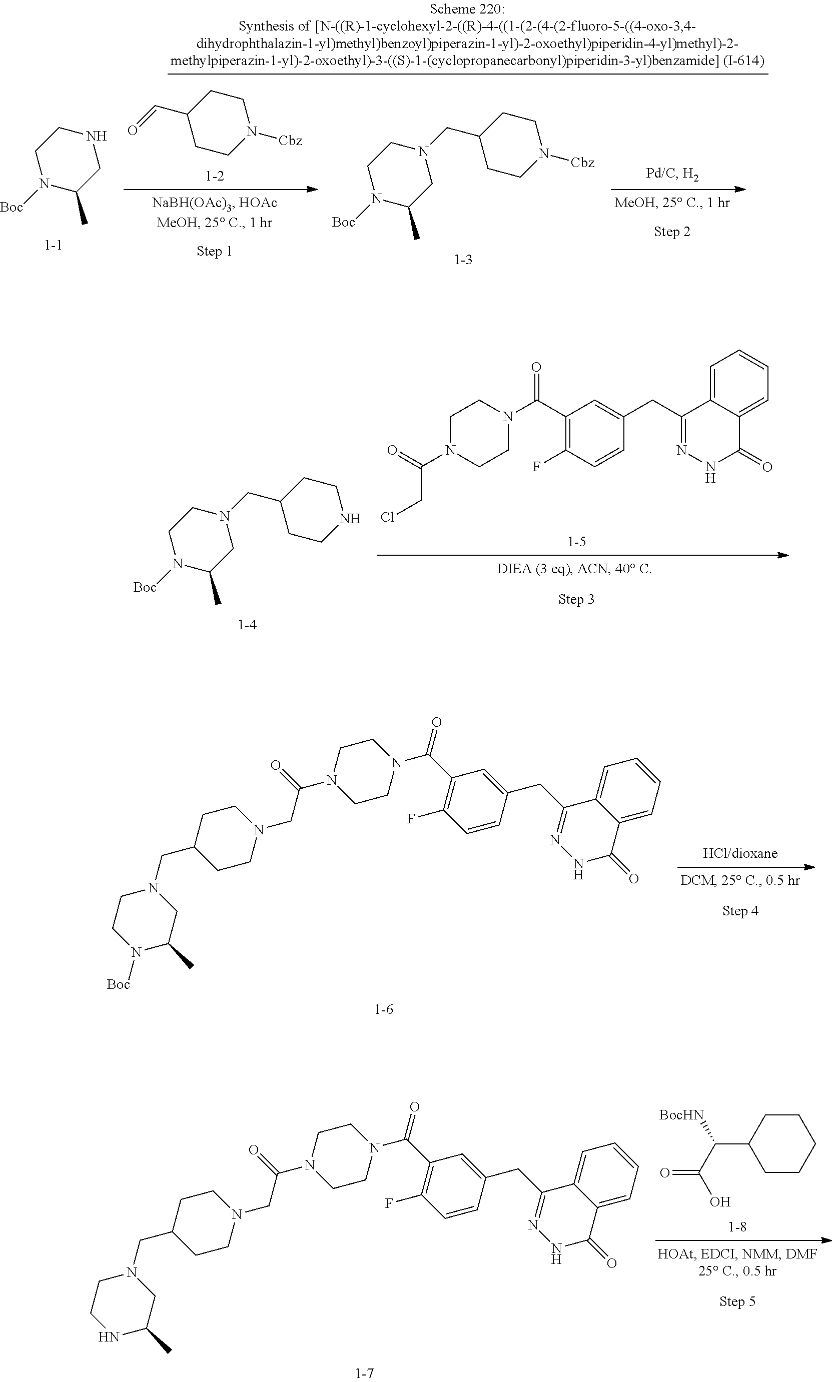

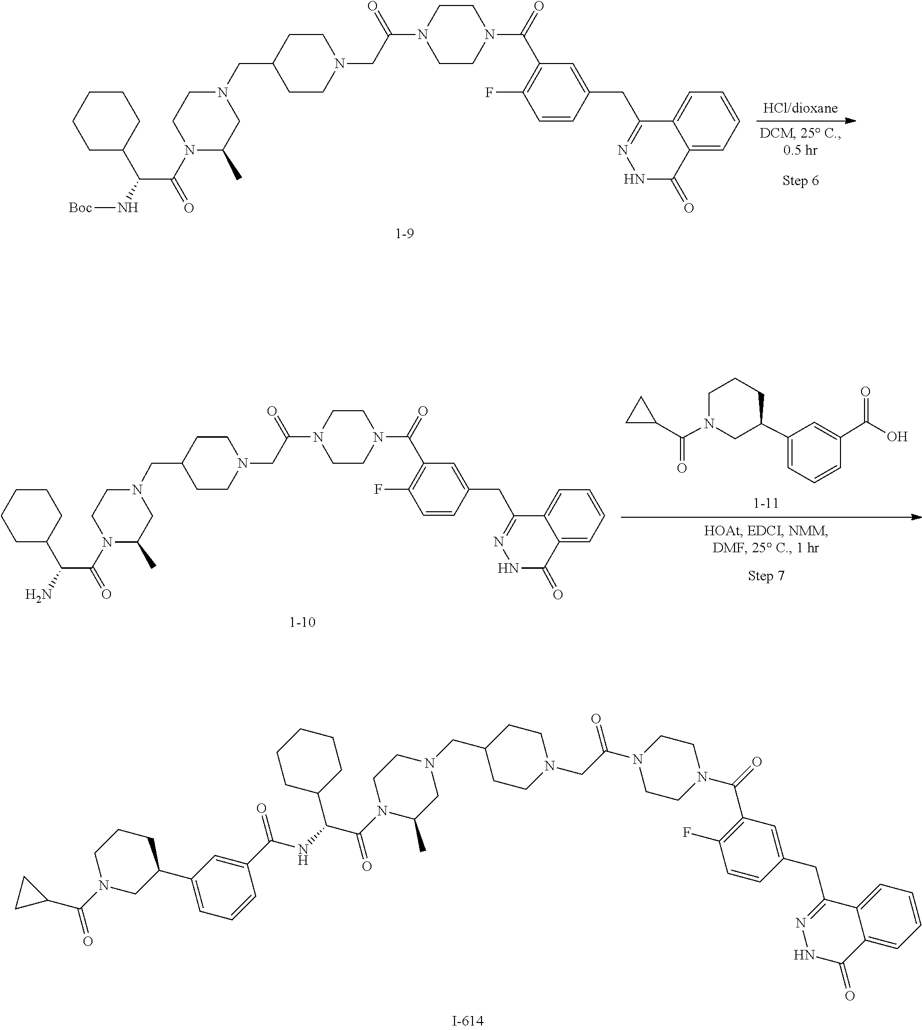 [SMILES: C[C@@H]1CN(CC2CCN(CC(=O)N3CCN(C(=O)c4cc(Cc5n[nH]c(=O)c6ccccc56)ccc4F)CC3)CC2)CCN1C(=O)[C@H](NC(=O)OC(C)(C)C)C1CCCCC1 ; C[C@@H]1CN(CC2CCN(CC(=O)N3CCN(C(=O)c4cc(Cc5n[nH]c(=O)c6ccccc56)ccc4F)CC3)CC2)CCN1C(=O)[C@H](N)C1CCCCC1 ; O=C(O)c1cccc([C@@H]2CCCN(C(=O)C3CC3)C2)c1 ; C[C@@H]1CN(CC2CCN(CC(=O)N3CCN(C(=O)c4cc(Cc5n[nH]c(=O)c6ccccc56)ccc4F)CC3)CC2)CCN1C(=O)[C@H](NC(=O)c1cccc([C@@H]2CCCN(C(=O)C3CC3)C2)c1)C1CCCCC1]

### Step 1: Synthesis of Intermediate 1-3.

To a solution of intermediate 1-1 (300 mg, 1.50 mmol, 1 eq) and intermediate 1-2 (370.42 mg, 1.50 mmol, 1 eq) in MeOH (3 mL) was added and AcOH (89.95 mg, 1.50 mmol, 85.75 μL, 1 eq) and the mixture was stirred for 0.1 hr. Then NaBH(OAc)₃ (634.94 mg, 3.00 mmol, 2 eq) was added into the mixture. The mixture was stirred at 25° C. for 1 hr. The reaction mixture was diluted with H₂O (5 mL) and extracted with EA (10 mL*3). The combined organic layers were dried over Na₂SO₄, filtered and concentrated under reduced pressure to give a residue. The residue was purified by flash silica gel chromatography (ISCO; 20 g SepaFlash Silica Flash Column, Eluent of 0˜20% Ethyl acetate/Petroleum ether gradient @60 mL/min), the eluent was concentrated under reduced pressure to give a residue. Intermediate 1-3 (550 mg, 1.27 mmol, 85.08% yield) was obtained as a yellow oil. LCMS (Method D): Retention time: 0.304 min, [M+H]⁺=432.7. ¹H NMR (400 MHz, METHANOL-d4) δ=7.43-7.20 (m, 5H), 4.22-4.05 (m, 4H), 3.77 (d, J=12.8 Hz, 1H), 3.14-3.03 (m, 1H), 2.89-2.66 (m, 4H), 2.20-2.09 (m, 2H), 2.02-1.97 (m, 6H), 1.78 (d, J=4.8 Hz, 2H), 1.45 (s, 9H), 1.23 (d, J=6.4 Hz, 3H).

### Step 2: Synthesis of Intermediate 1-4.

To a solution of Pd(OH)₂/C (300 mg, 427.24 μmol, 20% purity) in MeOH (5 mL) was added intermediate 1-3 (500 mg, 1.16 mmol, 1 eq), the mixture was then degassed and purged with H₂ 3 times, and then the mixture was stirred at 25° C. for 1 hr under H₂ (15 psi) atmosphere. The reaction mixture was filtered and washed with MeOH (15 mL*2), and the filtrate was concentrated under reduced pressure to give a product. The crude product was used in the next step without further purification. Intermediate 1-4 (350 mg, crude) was obtained as a colorless oil. LCMS (Method D): Retention time: 0.294 min, [M+H]⁺=298.2. ¹H NMR (400 MHz, METHANOL-d4) δ=4.16 (s, 1H), 3.76 (d, J=13.2 Hz, 1H), 3.19-3.03 (m, 3H), 2.79-2.63 (m, 4H), 2.21-2.03 (m, 3H), 1.99-1.81 (m, 4H), 1.74 (d, J=2.8 Hz, 1H), 1.45 (s, 9H), 1.24 (d, J=6.0 Hz, 4H).

### Step 3: Synthesis of Intermediate 1-6.

To a solution of intermediate 1-4 (350 mg, 1.18 mmol, 1 eq) and intermediate 1-5 (521.14 mg, 1.18 mmol, 1 eq) in ACN (4 mL) was added DIEA (456.25 mg, 3.53 mmol, 614.89 μL, 3 eq). The mixture was stirred at 40° C. for 0.5 hr. The reaction mixture was concentrated under reduced pressure to give a residue. The residue was purified by flash silica gel chromatography (ISCO; 20 g SepaFlash Silica Flash Column, Eluent of 0˜5% Methanol/Ethyl acetate gradient @60 mL/min), the eluent was concentrated under reduced pressure to give a residue. Intermediate 1-6 (400 mg, 516.38 μmol, 43.88% yield, 90.863% purity) was obtained as a yellow solid. LCMS (Method D): Retention time: 0.275 min, [M+H]⁺=704.3.1H NMR (400 MHz, METHANOL-d4) δ=8.36 (d, J=8.0 Hz, 1H), 7.94 (d, J=7.2 Hz, 1H), 7.89-7.77 (m, 2H), 7.47 (s, 1H), 7.41-7.35 (m, 1H), 7.19-7.11 (m, 1H), 4.37 (s, 2H), 4.19-4.12 (m, 1H), 3.94-3.59 (m, 6H), 3.59-3.49 (m, 2H), 3.36-3.32 (m, 3H), 3.24 (s, 1H), 3.20-3.16 (m, 1H), 3.12-3.02 (m, 1H), 2.95-2.83 (m, 2H), 2.77-2.62 (m, 2H), 2.19-2.03 (m, 4H), 1.94-1.85 (m, 1H), 1.75 (d, J=10.8 Hz, 2H), 1.45 (s, 9H), 1.28-1.16 (m, 5H).

### Step 4: Synthesis of Intermediate 1-7.

To a solution of intermediate 1-6 (300 mg, 426.23 μmol, 1 eq) in DCM (2 mL) was added HCl/dioxane (2 M, 2 mL) and it was stirred at 25° C. for 0.1 hr. The reaction mixture was concentrated under reduced pressure to give a residue. The crude product was used in the next step without further purification. Intermediate 1-7 (280 mg, crude, HCl salt) was obtained as a white solid. LCMS (Method D): Retention time: 0.252 min, (M+H)=604.2.

### Step 5: Synthesis of Intermediate 1-9.

To a solution of intermediate 1-7 (250 mg, 390.51 μmol, 1 eq, HCl salt) and intermediate 1-8 (100.49 mg, 390.51 μmol, 1 eq) in DMF (3 mL) was added HOAt (53.15 mg, 390.51 μmol, 54.63 μL, 1 eq), EDCI (224.58 mg, 1.17 mmol, 3 eq) and NMM (197.49 mg, 1.95 mmol, 214.67 μL, 5 eq). The mixture was stirred at 25° C. for 0.5 hr. The reaction mixture was diluted with H₂O (5 mL) and extracted with EA (10 mL*3). The combined organic layers were washed with brine (10 mL*3), dried over Na₂SO₄, filtered and concentrated under reduced pressure to give a residue. The residue was purified by flash silica gel chromatography (ISCO; 12 g SepaFlash Silica Flash Column, Eluent of 0˜5% Methanol/Ethyl acetate gradient @60 mL/min), and the eluent was concentrated under reduced pressure to give a residue. Intermediate 1-9 (150 mg, 174.53 μmol, 44.69% yield, 98.090% purity) was obtained as a yellow solid. LCMS (Method D): Retention time: 0.331 min, [M+H]⁺=843.3. SFC: Retention time: 1.019 min. ¹H NMR (400 MHz, CHLOROFORM-d) δ=8.51-8.40 (m, 1H), 7.86-7.70 (m, 3H), 7.33 (d, J=4.8 Hz, 2H), 7.12-7.02 (m, 1H), 5.34-5.18 (m, 1H), 4.71-4.59 (m, 1H), 4.45-4.37 (m, 1H), 4.29 (s, 2H), 3.89-3.54 (m, 8H), 3.39 (d, J=9.6 Hz, 2H), 3.33-3.25 (m, 1H), 3.03-2.90 (m, 1H), 2.82-2.59 (m, 3H), 2.23-2.06 (m, 3H), 1.90-1.51 (m, 13H), 1.43 (s, 9H), 1.38 (s, 2H), 1.30-1.20 (m, 4H), 1.18-1.11 (m, 2H), 1.03 (d, J=9.2 Hz, 3H). ¹⁹F NMR (376 MHz, CHLOROFORM-d) δ=−117.618(s).

### Step 6: Synthesis of Intermediate 1-10.

To a solution of intermediate 1-9 (150 mg, 177.93 μmol, 1 eq) in DCM (0.5 mL) was added HCl/dioxane (2 M, 0.5 mL). The mixture was stirred at 25° C. for 0.5 hr. The reaction mixture was concentrated under reduced pressure to give a residue. The crude product was used in the next step without further purification. Intermediate 1-10 (130 mg, 166.80 μmol, 93.74% yield, HCl salt) was obtained as a white solid. LCMS (Method D): Retention time: 0.249 min, [M+H]⁺=1265.6.

### Step 7: Synthesis of I-614

To a solution of intermediate 1-10 (60 mg, 76.98 μmol, 1 eq, HCl salt) and intermediate 1-11 (21.04 mg, 76.98 μmol, 1 eq) in DMF (1 mL) was added HOAt (10.48 mg, 76.98 μmol, 10.77 μL, 1 eq), EDCI (44.27 mg, 230.95 μmol, 3 eq) and NMM (38.93 mg, 384.92 μmol, 42.32 μL, 5 eq). The mixture was stirred at 25° C. for 1 hr. The reaction mixture was diluted with H₂O 5 (mL) and extracted with EA (10 mL*3). The combined organic layers were washed with brine (10 mL*3), dried over Na₂SO₄, filtered and concentrated under reduced pressure to give a residue. The crude product was purified by reversed phase HPLC (neutral condition), and the eluent was concentrated and lyophilized to give the desired product. I-614 (15.2 mg, 14.99 μmol, 19.47% yield, 98.457% purity) was obtained as a white solid. LCMS (Method D): Retention time: 0.361 min, [M+H]⁺=998.4. HPLC: Retention time: 2.787 minSFC: Retention time: 2.282 min. ¹H NMR (400 MHz, METHANOL-d4) δ=8.37 (d, J=7.6 Hz, 1H), 7.99-7.93 (m, 1H), 7.89-7.80 (m, 2H), 7.78-7.66 (m, 2H), 7.53-7.34 (m, 4H), 7.20-7.13 (m, 1H), 4.68-4.53 (m, 2H), 4.44-4.29 (m, 4H), 4.07-3.95 (m, 1H), 3.82-3.65 (m, 4H), 3.60-3.45 (m, 3H), 3.24-3.02 (m, 3H), 2.96-2.85 (m, 3H), 2.80-2.68 (m, 3H), 2.24-1.96 (m, 9H), 1.92-1.66 (m, 11H), 1.62-1.41 (m, 3H), 1.34-1.19 (m, 7H), 1.17-1.05 (m, 2H), 0.91-0.78 (m, 4H). ¹⁹F NMR (376 MHz, METHANOL-d4) δ=−120.79.

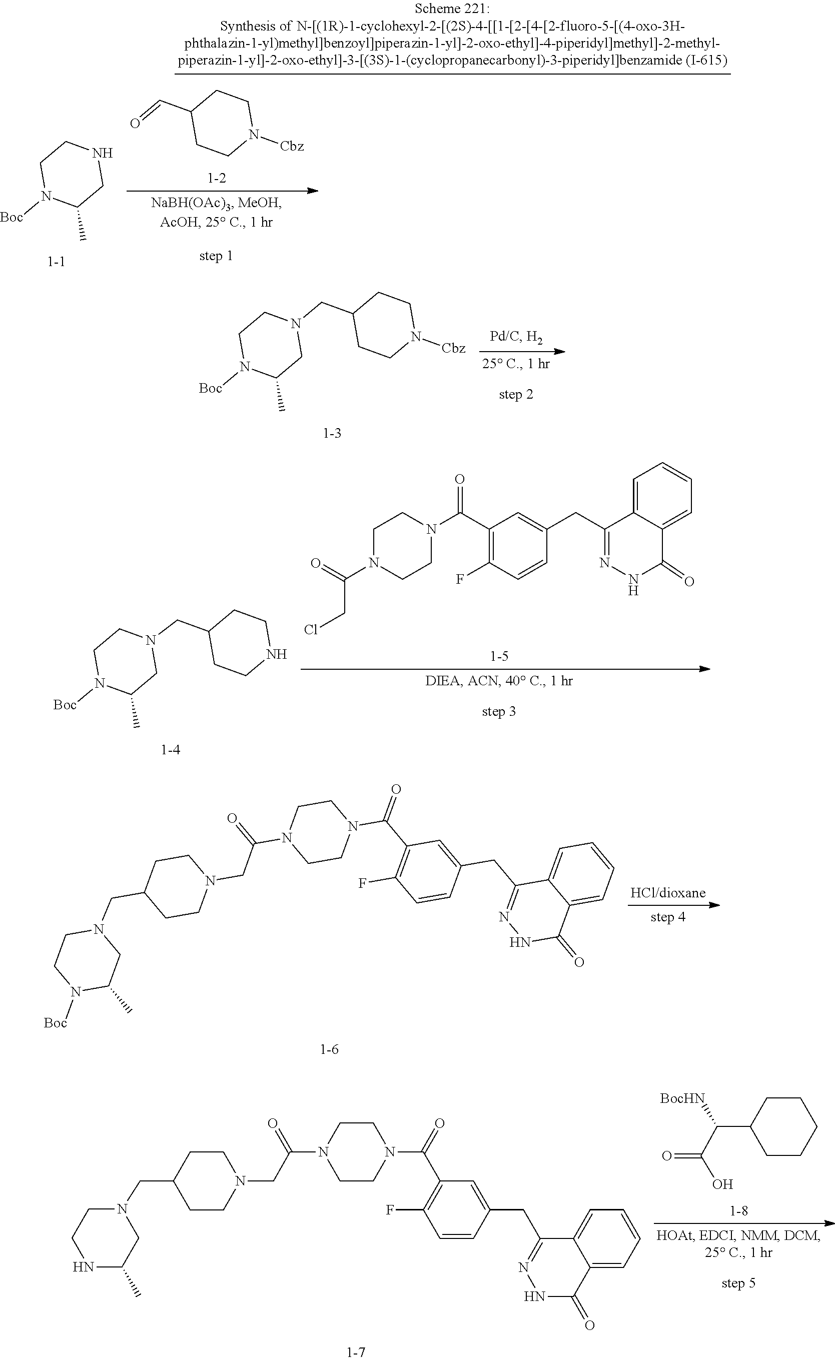

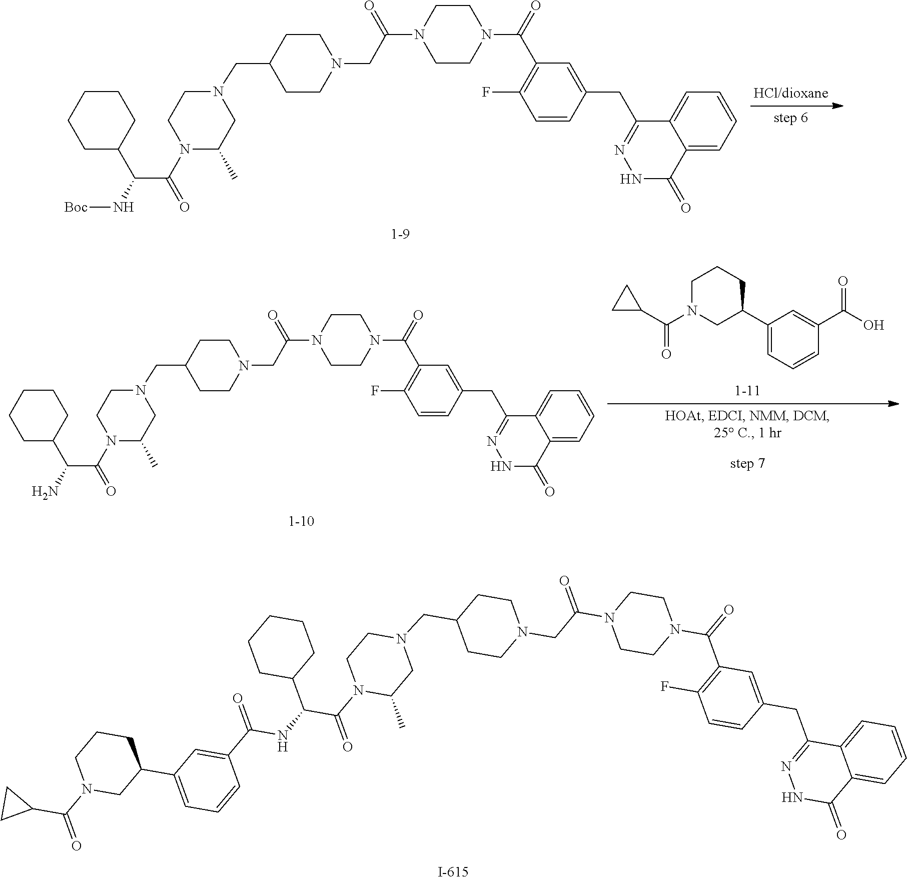 [SMILES: C[C@H]1CN(CC2CCN(CC(=O)N3CCN(C(=O)c4cc(Cc5n[nH]c(=O)c6ccccc56)ccc4F)CC3)CC2)CCN1C(=O)[C@H](NC(=O)OC(C)(C)C)C1CCCCC1 ; C[C@H]1CN(CC2CCN(CC(=O)N3CCN(C(=O)c4cc(Cc5n[nH]c(=O)c6ccccc56)ccc4F)CC3)CC2)CCN1C(=O)[C@H](N)C1CCCCC1 ; O=C(O)c1cccc([C@@H]2CCCN(C(=O)C3CC3)C2)c1 ; C[C@H]1CN(CC2CCN(CC(=O)N3CCN(C(=O)c4cc(Cc5n[nH]c(=O)c6ccccc56)ccc4F)CC3)CC2)CCN1C(=O)[C@H](NC(=O)c1cccc([C@@H]2CCCN(C(=O)C3CC3)C2)c1)C1CCCCC1]

### Step 1: Synthesis of Intermediate 1-3

To solution of intermediate 1-1 (500 mg, 2.50 mmol, 1 eq) and intermediate 1-2 (802.58 mg, 3.25 mmol, 1.3 eq) in MeOH (8 mL) was added AcOH (299.84 mg, 4.99 mmol, 285.84 μL, 2 eq). The mixture was stirred at 25° C. for 0.5 h. Then NaBH(OAc)₃ (2.65 g, 12.48 mmol, 5 eq) was added into the mixture and it was stirred at 25° C. for 0.5 h. The mixture was concentrated under reduced pressure to give a residue. Then the reaction mixture was diluted with water (60 mL) and extracted with EA (20 mL×3). The combined organic layers were washed with brine (20 mL×3) and dried over sodium sulfate, filtered and concentrated under reduced pressure to give a residue. The residue was purified by flash silica gel chromatography (ISCO; 40 g SepaFlash Silica Flash Column, Eluent of 0˜40% EA:PE gradient @40 mL/min). The eluent was concentrated under reduced pressure to give intermediate 1-3 (650 mg, 1.16 mmol, 46.49% yield, 77.06% purity), which was obtained as a yellow oil. LCMS (Method D): Rt: 0.332 min, [M+H]⁺=432.2.

### Step 2: Synthesis of Intermediate 1-4

To a solution of intermediate 1-3 (500 mg, 1.16 mmol, 1 eq) in MeOH (5 mL) was added Pd(OH)₂/C (300.00 mg). The suspension was degassed and purged with N₂ 3 times. The mixture was stirred at 25° C. for 1 h under H₂ (15 Psi) atmosphere. The reaction mixture was filtered and washed with MeOH (3 mL*5), the filtrate was concentrated under reduced pressure to give intermediate 1-4 (320 mg, 1.08 mmol, 92.86% yield) as a yellow oil. LCMS (Method D): Rt: 0.366 min, [M+H]⁺=298.2.

### Step 3: Synthesis of Intermediate 1-6

To a solution of intermediate 1-4 (300 mg, 1.01 mmol, 1 eq) and intermediate 1-5 (446.69 mg, 1.01 mmol, 1 eq) in ACN (8 mL) was added DIEA (391.07 mg, 3.03 mmol, 527.05 μL, 3 eq). The mixture was stirred at 40° C. for 1 h. The mixture was concentrated under reduced pressure to give a residue. Then the reaction mixture was diluted with water (60 mL) and extracted with ethyl acetate (20 mL×3). The combined organic layers were washed with brine (20 mL×3) and dried over sodium sulfate, filtered and concentrated under reduced pressure to give a residue. The residue was purified by flash silica gel chromatography (ISCO®; 40 g SepaFlash® Silica Flash Column, Eluent of 0˜30% Methanol: Ethyl acetate gradient @40 mL/min). The eluent was concentrated under reduced pressure to give intermediate 1-6 (610 mg, 755.52 μmol, 74.91% yield, 87.175% purity) as a yellow oil. LCMS (Method D): Rt: 0.283 min, [M+H]⁺=704.2. ¹H NMR (400 MHz, CHLOROFORM-d) δ=10.52-10.34 (m, 1H), 8.52-8.39 (m, 1H), 7.83-7.67 (m, 3H), 7.40-7.30 (m, 2H), 7.05 (s, 1H), 4.29 (s, 2H), 4.18 (d, J=1.2 Hz, 1H), 3.85-3.69 (m, 4H), 3.64-3.53 (m, 2H), 3.49 (s, 11H), 3.37-3.21 (m, 3H), 3.12-3.01 (m, 1H), 2.88 (d, J=6.8 Hz, 1H), 2.69 (d, J=8.4 Hz, 1H), 2.56 (d, J=10.4 Hz, 1H), 2.20-2.05 (m, 4H), 1.94-1.93 (m 1H), 1.76 (s, 3H), 1.24-1.13 (m, 7H). ¹⁹F NMR (376 MHz, METHANOL-d4) δ=−117.634 (s). SFC: Rt: 1.283 min.

### Step 4: Synthesis of Intermediate 1-7

To a solution of intermediate 1-6 (550 mg, 781.42 μmol, 1 eq) in DCM (5 mL) was added HCl/dioxane (2 M, 195.36 L). The mixture was stirred at 25° C. for 0.5 h. The mixture was concentrated under reduced pressure to give intermediate 1-7 (450 mg, 745.37 μmol, 95.39% yield) as a yellow oil. LCMS (Method D): Rt: 0.221 min, [M+H]⁺=604.3.

### Step 5: Synthesis of Intermediate 1-9

To a solution of intermediate 1-8 (170.49 mg, 662.55 μmol, 1 eq) in DCM (7 mL) was added EDCI (381.03 mg, 1.99 mmol, 3 eq), NMM (335.07 mg, 3.31 mmol, 364.21 μL, 5 eq) and HOAt (90.18 mg, 662.55 μmol, 92.68 μL, 1 eq) and it was stirred at 25° C. for 0.5 hr. And then intermediate 1-7 (400 mg, 662.55 μmol, 1 eq) was added into mixture and stirred at 25° C. for 0.5 hr. The reaction mixture was diluted with water (60 mL) and extracted with DCM (20 mL×3). The combined organic layers were washed with brine (20 mL×3) and dried over sodium sulfate, filtered and concentrated under reduced pressure to give a residue. The residue was purified by flash silica gel chromatography (ISCO; 40 g SepaFlash Silica Flash Column, Eluent of 0˜30% Methanol:Ethyl acetate gradient @40 mL/min). The eluent was concentrated under reduced pressure to give intermediate 1-9 (500 mg, 555.04 μmol, 83.77% yield, 93.584% purity) as a yellow oil. LCMS (Method D): Rt: 0.325 min, [M+H]⁺=843.3. ¹H NMR (400 MHz, CHLOROFORM-d) δ=10.64-10.49 (m, 1H), 8.52-8.42 (m, 1H), 7.83-7.66 (m, 3H), 7.42-7.29 (m, 2H), 7.05 (s, 1H), 4.46-4.44 (m, 1H), 4.29 (s, 2H), 3.76-3.74 (m, 9H), 3.66-3.54 (m, 2H), 3.48 (s, 2H), 3.35 (bs, 1H), 3.22-3.14 (m, 2H), 2.90-2.74 (m, 3H), 2.48 (s, 7H), 2.34 (s, 6H), 2.18-2.02 (m, 5H), 1.75 (s, 4H), 1.67-1.60 (m, 2H), 1.36 (d, J=6.0 Hz, 1H), 1.28-1.00 (m, 9H). ¹⁹F NMR (376 MHz, METHANOL-d4) δ=−117.664 (s). SFC: Rt: 1.341 min.

### Step 6: Synthesis of Intermediate 1-10

To a solution of intermediate 1-9 (200 mg, 237.24 μmol, 1 eq) in DCM (2 mL) was added HCl/dioxane (2 M, 1 mL). The mixture was stirred at 25° C. for 0.5 h. The mixture was concentrated under reduced pressure to give intermediate 1-10 (150 mg, crude, HCl salt) as a yellow oil. LCMS (Method D): Rt: 0.244 min, [M+H]⁺=743.4.

### Step 7: Synthesis of I-615

To a solution of intermediate 1-11 (35.07 mg, 128.31 μmol, 1 eq) in DCM (1.5 mL) was added EDCI (73.79 mg, 384.92 μmol, 3 eq), NMM (64.89 mg, 641.53 μmol, 70.53 μL, 5 eq) and HOAt (17.46 mg, 128.31 μmol, 17.95 μL, 1 eq) at 25° C. for 0.5 h. Then, intermediate 1-10 (100 mg, 128.31 μmol, 1 eq, HCl) was added into mixture and it was stirred at 25° C. for 0.5 h. The reaction mixture was diluted with water (30 mL) and extracted with EA (10 mL×3). The combined organic layers were washed with brine (10 mL×3) and dried over sodium sulfate, filtered and concentrated under reduced pressure to give a residue. The residue was purified by prep-HPLC (column: (s,s) WHELK-01 (250 mm*30 mm, 10 um); mobile phase: [CO₂-EtOH (0.1% NH₃H2O)]; B%: 60%, isocratic elution mode) and the eluent was concentrated under reduced pressure to remove ACN and then lyophilized to give I-615 (5.68 mg, 5.60 μmol, 4.37% yield, 98.454% purity) as a white solid. LCMS (Method D): Rt: 0.353 min, [M+H]⁺=998.3. ¹H NMR (400 MHz, METHANOL-d4) δ=8.37 (d, J=7.6 Hz, 1H), 7.98-7.92 (m, 1H), 7.91-7.82 (m, 2H), 7.81-7.67 (m, 2H), 7.55-7.32 (m, 4H), 7.16 (s, 1H), 5.03-4.90 (m, 1H), 4.77-4.68 (m, 1H), 4.57 (d, J=13.2 Hz, 1H), 4.44-4.25 (m, 4H), 3.83-3.64 (m, 4H), 3.61-3.43 (m, 3H), 3.29-3.17 (m, 3H), 2.98-2.79 (m, 4H), 2.79-2.66 (m, 2H), 2.26-1.97 (m, 8H), 1.96-1.62 (m, 12H), 1.61-1.40 (m, 3H), 1.36-1.18 (m, 7H), 1.16-1.02 (m, 2H), 0.95-0.73 (m, 4H). ¹⁹F NMR (376 MHz, METHANOL-d4) δ=−120.716 (s). SFC: Rt: 2.020 min.

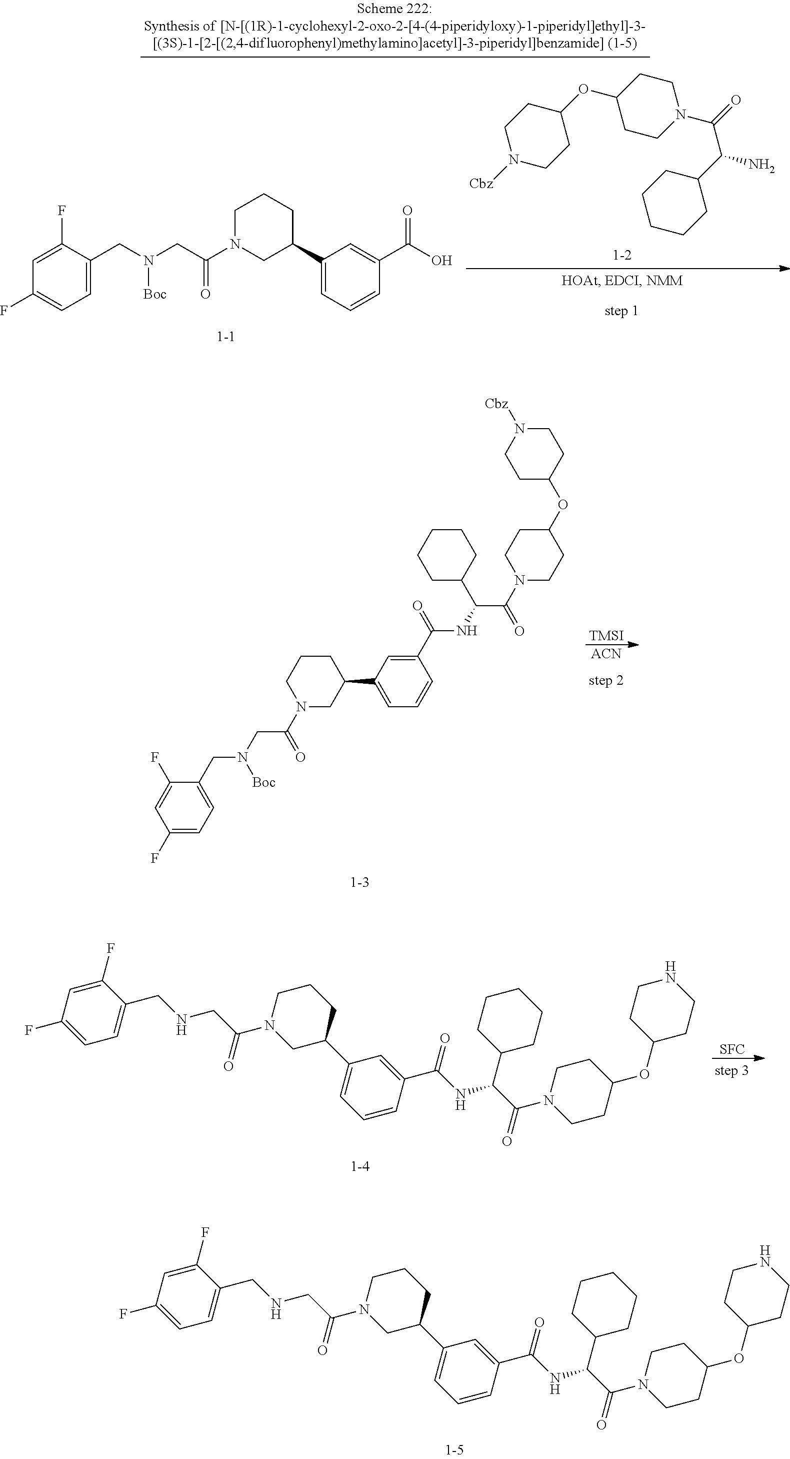

### Step 1: Synthesis of Intermediate 1-3

To a solution of intermediate 1-1 (200 mg, 404.81 μmol, 1 eq, HCl salt) in DMF (2 mL) was added EDCI (232.80 mg, 1.21 mmol, 3 eq), NMM (204.72 mg, 2.02 mmol, 222.53 μL, 5 eq), intermediate 1-2 (197.76 mg, 404.81 μmol, 1 eq) and HOAt (55.10 mg, 404.81 μmol, 56.63 μL, 1 eq). The mixture was stirred at 25° C. for 0.5 h. The mixture was purified by reversed phase HPLC (0.1% FA condition) and the eluent was lyophilized to give intermediate 1-3 (220 mg, 227.56 μmol, 56.21% yield, 96% purity) as a yellow solid. SFC: Retention time: 0.587 min, 1.837 min. LCMS (Method D): Retention time: 0.585 min, [M+H]⁺=928.6.

### Step 2: Synthesis of Intermediate 1-4

To Intermediate 1-3 (200 mg, 215.49 μmol, 1 eq) in ACN (2 mL) was added TMSI (86.24 mg, 430.98 μmol, 58.66 μL, 2 eq). The mixture was stirred at 25° C. for 1 h. The mixture was purified by prep-HPLC (column: CD01-Phenomenex luna C18 150*25*10 um; mobile phase: [water(FA)-ACN]; gradient: 9%-39% B over 10 min) and the eluent was lyophilized to give intermediate 1-4 (50 mg, 70.62 μmol, 32.77% yield, 98% purity) as a white solid. SFC: Retention time: 3.582 min, 4.749 min. LCMS (Method D): Retention time: 0.368 min, [M+H]⁺=694.4. ¹H NMR (400 MHz, METHANOL-d4) δ=8.46 (s, 1H), 7.80-7.68 (m, 2H), 7.61-7.39 (m, 3H), 7.13-6.95 (m, 2H), 4.98-4.93 (m, 1H), 4.62-4.44 (m, 1H), 4.11 (d, J=15.2 Hz, 2H), 4.07-3.90 (m, 3H), 3.89-3.58 (m, 5H), 3.57-3.45 (m, 1H), 3.39-3.33 (m, 1H), 3.30-3.17 (m, 2H), 3.15-3.11 (m, 2H), 3.01-2.71 (m, 2H), 2.04-2.00 (m, 4H), 1.94-1.75 (m, 9H), 1.74-1.44 (m, 6H), 1.37-1.18 (m, 3H), 1.18-1.04 (m, 2H).

### Step 3: Synthesis of Intermediate 1-5

Intermediate 1-4 (50 mg) was purified by SFC silica gel chromatography (column: DAICEL CHIRALPAK IK (250 mm*30 mm, 10 um); mobile phase: [Hexane-IPA (0.1% NH₃)]; B%: 40%, isocratic elution mode) and concentrated under reduced pressure to give product. Intermediate 1-5 (21.17 mg, 30.08 μmol, 41.75% yield, 98.601% purity) was obtained as a yellow solid. SFC: Retention time: 3.716 min. LCMS (Method D): Retention time: 0.310 min, [M+H]⁺=694.4. ¹H NMR (400 MHz, METHANOL-d4) δ=9.41 (s, 1H), 8.79 (s, 1H), 8.43 (d, J=4.0 Hz, 1H), 8.38-8.35 (m, 1H), 7.98-7.91 (m, 1H), 7.91-7.80 (m, 3H), 7.56-7.46 (m, 5H), 7.41-7.34 (m, 1H), 7.16 (t, J=9.2 Hz, 1H), 5.82 (s, 2H), 4.56-4.45 (m, 1H), 4.38 (s, 2H), 3.94 (s, 3H), 3.80-3.64 (m, 4H), 3.56-3.48 (m, 2H), 3.30-3.18 (m, 4H), 3.13 (t, J=6.4 Hz, 2H), 2.84 (d, J=9.6 Hz, 2H), 2.61-2.37 (m, 10H), 1.93-1.81 (m, 2H), 1.42 (d, J=6.8 Hz, 6H). ¹⁹F NMR: (377 MHz, METHANOL-d₄) δ=−116.275 (s), −113.453 (s).

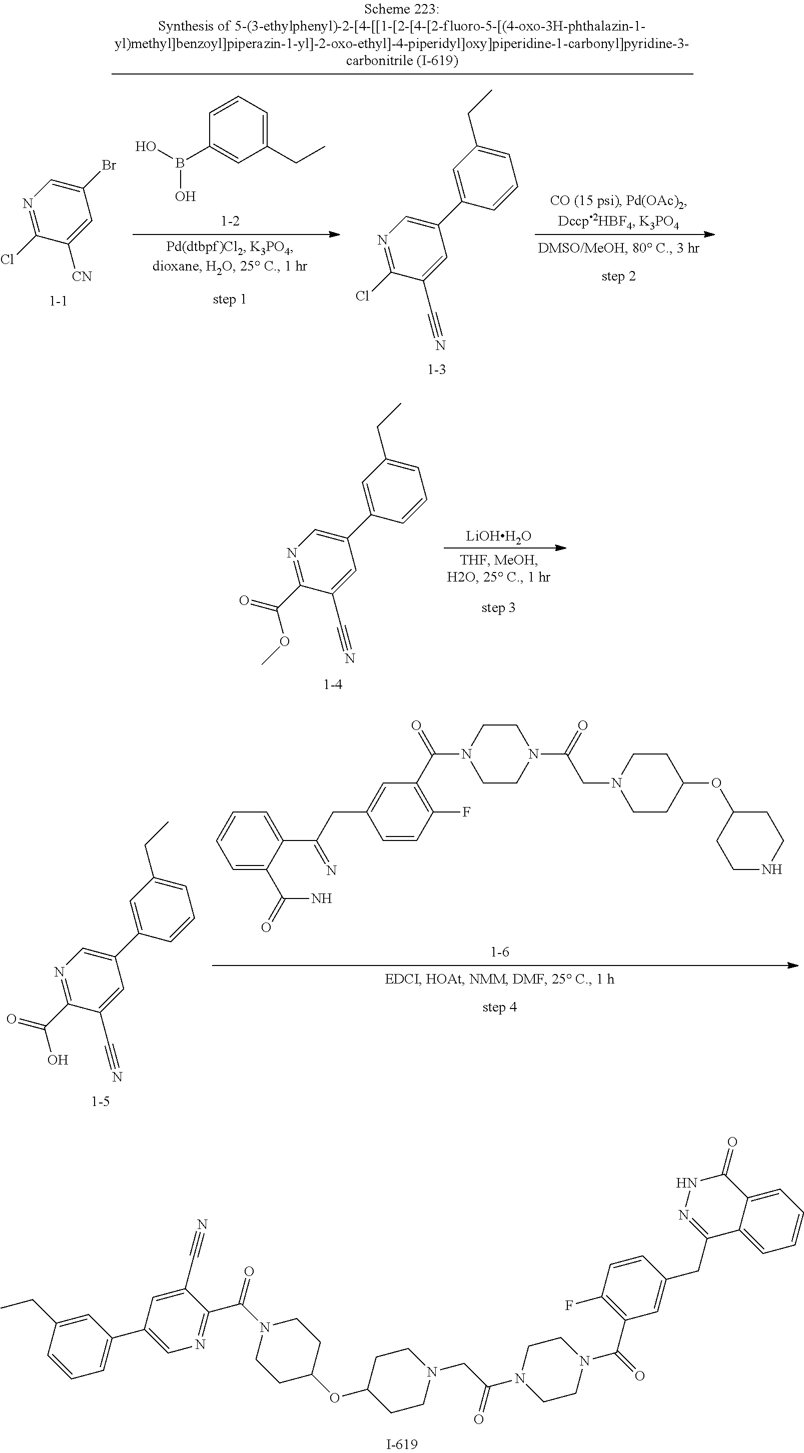

### Step 1: Synthesis of Intermediate 1-3

To a solution of intermediate 1-1 (600 mg, 2.76 mmol, 1 eq) and intermediate 1-2 (455.22 mg, 3.04 mmol, 1.1 eq) in dioxane (6 mL) and H₂O (1.2 mL) was added Pd(dtbpf)Cl₂ (179.83 mg, 275.92 μmol, 0.1 eq) and K₃PO₄ (1.76 g, 8.28 mmol, 3 eq). The mixture was stirred at 80° C. for 2 hr. The reaction mixture was extracted with DCM (5 mL*3). The combined organic layers were dried over Na₂SO₄, filtered and concentrated under reduced pressure to give a residue. The residue was purified by flash silica gel chromatography (ISCO; 40 g SepaFlash Silica Flash Column, Eluent of 0˜9% Ethyl acetate/Petroleum ether gradient @60 mL/min) and concentrated under reduced pressure to give a residue. Intermediate 1-3 (750 mg, crude) was obtained as a yellow solid. LCMS (Method D): Retention time:=0.483 min, [M+H]⁺=242.9.

### Step 2: Synthesis of Intermediate 1-4

To a solution of intermediate 1-3 (650 mg, 2.68 mmol, 1 eq) in DMSO (7 mL) and MeOH (7 mL) was added K₃PO₄ (5.36 mmol, 2 eq), Pd(OAc)₂ (60.13 mg, 267.82 μmol, 0.1 eq), Dccp·HBF₄ (535.63 mol, 0.2 eq). Then the mixture was stirred at 80° C. for 3 hr under CO atmosphere. The reaction mixture was filtered and concentrated under reduced pressure to give a residue. The crude product was used in the next step without further purification. Intermediate 1-4 (600 mg, 2.25 mmol, 84.13% yield) was obtained as a white solid. LCMS (Method D): Retention time:=0.428 min, [M+H]⁺=267.2.

### Step 3: Synthesis of Intermediate 1-5

To a solution of intermediate 1-4 (150 mg, 563.29 μmol, 1 eq) in THF (1.5 mL), MeOH (1.5 mL), and H₂O (0.75 mL) was added LiOH·H₂O (118.19 mg, 2.82 mmol, 5 eq). The mixture was stirred at 25° C. for 1 hr. The reaction mixture was concentrated under reduced pressure and adjusted pH=5 with HCl (1 M). Then the mixture was extracted with DCM (10 mL*3). The combined organic layers were dried over Na₂SO₄, filtered and concentrated under reduced pressure to give a residue. The crude product was used in the next step without further purification. Intermediate 1-5 (182 mg, crude) was obtained as a white solid. LCMS (Method D): Retention time:=0.348 min, [M+H]⁺=253.1.

### Step 4: Synthesis of I-619

To a solution of intermediate 1-5 (132 mg, 523.25 μmol, 1 eq) and intermediate 1-6 (328.16 mg, 523.25 μmol, 1 eq, HCl salt) in DMF (1.5 mL) was added EDCI (300.92 mg, 1.57 mmol, 3 eq), NMM (264.63 mg, 2.62 mmol, 287.64 μL, 5 eq) and HOAt (71.22 mg, 523.25 μmol, 73.20 μL, 1 eq). The mixture was stirred at 25° C. for 1 hr. The reaction mixture was filtered and concentrated under reduced pressure to give a residue. The crude product was purified by reversed phase HPLC (0.1% FA condition) followed by lyophilization to give product. I-619 (157.5 mg, 176.83 μmol, 33.79% yield, 97.786% purity, FA salt) was obtained as a white solid. LCMS (Method D): Retention time:=0.374 min, [M+H]⁺=825.3. ¹H NMR (400 MHz, METHANOL-d₄) δ=9.08 (d, J=1.6 Hz, 1H), 8.57 (d, J=2.0 Hz, 1H), 8.46 (s, 1H), 8.36 (d, J=7.6 Hz, 1H), 7.99-7.91 (m, 1H), 7.91-7.79 (m, 2H), 7.60 (s, 1H), 7.56 (d, J=7.6 Hz, 1H), 7.53-7.42 (m, 2H), 7.41-7.32 (m, 2H), 7.16 (t, J=8.8 Hz, 1H), 4.38 (s, 2H), 4.07 (d, J=6.8 Hz, 1H), 3.82 (d, J=12.0 Hz, 2H), 3.75 (s, 1H), 3.67 (s, 5H), 3.60-3.47 (m, 4H), 3.44-3.33 (m, 2H), 3.29-3.21 (m, 1H), 3.03 (s, 2H), 2.78-2.72 (m, 4H), 2.04-1.86 (m, 4H), 1.83-1.61 (m, 4H), 1.29 (t, J=7.6 Hz, 3H). ¹⁹F NMR (377 MHz, METHANOL-d₄), δ=−120.747 (s).

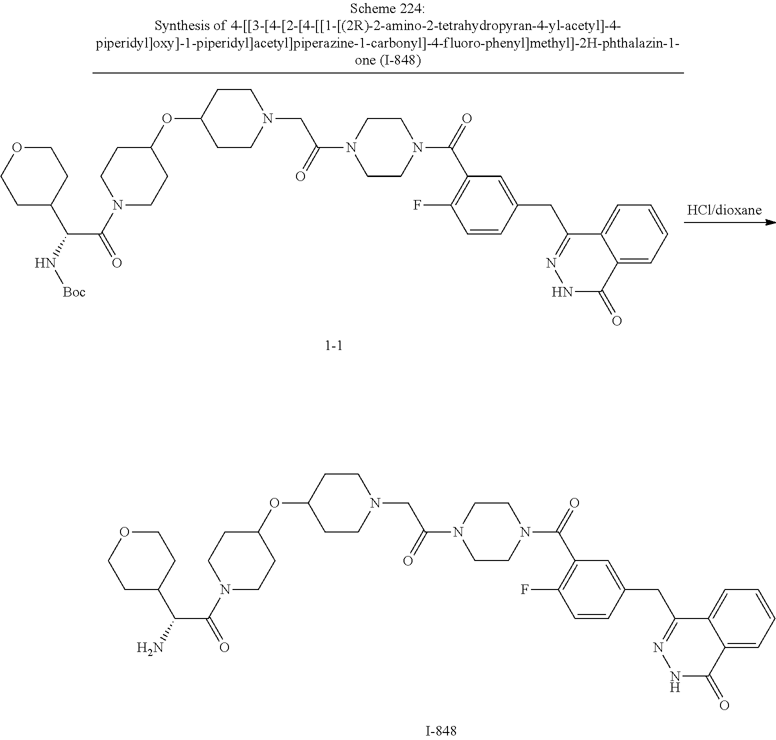 [SMILES: CC(C)(C)OC(=O)N[C@@H](C(=O)N1CCC(OC2CCN(CC(=O)N3CCN(C(=O)c4cc(Cc5n[nH]c(=O)c6ccccc56)ccc4F)CC3)CC2)CC1)C1CCOCC1 ; N[C@@H](C(=O)N1CCC(OC2CCN(CC(=O)N3CCN(C(=O)c4cc(Cc5n[nH]c(=O)c6ccccc56)ccc4F)CC3)CC2)CC1)C1CCOCC1]

A solution of intermediate 1-1 (50 mg, 60.10 μmol, 1 eq) in HCl/dioxane (2 M, 0.5 mL, 16.64 eq) was stirred at 25° C. for 1 hr. The reaction mixture was concentrated under reduced pressure to give a residue. The residue was purified by prep-HPLC (column: CD03-Welch Xtimate C18 150*25*5 um; mobile phase: [water(HCl)-ACN]; gradient: 1%-28% B over 10 min). The eluent was lyophilized to give product. I-848 (25 mg, 32.06 μmol, 53.35% yield, 98.534% purity, HCl salt) was obtained as a white solid. LCMS (Method D): Retention time:=0.255 min, [M+H]⁺=732.5. ¹H NMR (400 MHz, METHANOL-d4) δ=8.37 (d, J=7.6 Hz, 1H), 8.00-7.93 (m, 1H), 7.92-7.79 (m, 2H), 7.56-7.46 (m, 1H), 7.40-7.36 (m 1H), 7.17 (t, J=9.2 Hz, 1H), 4.44-4.27 (m, 5H), 4.10-3.94 (m, 3H), 3.91-3.70 (m, 6H), 3.69-3.62 (m, 1H), 3.59-3.42 (m, 5H), 3.42-3.33 (m, 6H), 3.22-3.12 (m, 1H), 2.23 (d, J=13.6 Hz, 1H), 2.07 (s, 3H), 2.00-1.78 (m, 3H), 1.72-1.47 (m, 6H). ¹⁹F NMR (377 MHz, METHANOL-d₄), δ=−120.764 (s). SFC: Retention time: 1.682 min.

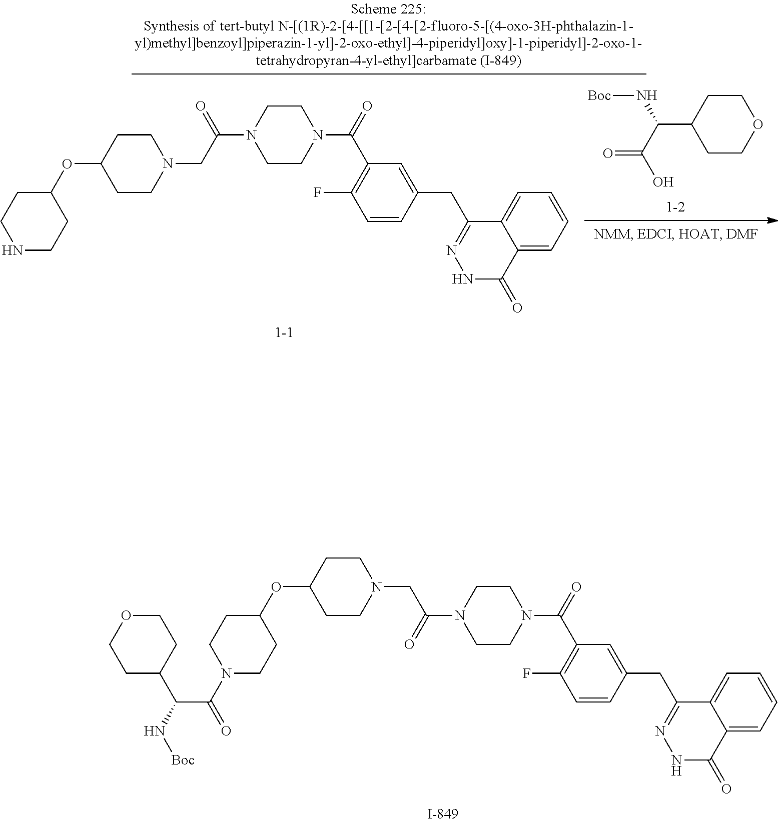 [SMILES: O=C(CN1CCC(OC2CCNCC2)CC1)N1CCN(C(=O)c2cc(Cc3n[nH]c(=O)c4ccccc34)ccc2F)CC1 ; CC(C)(C)OC(=O)N[C@@H](C(=O)O)C1CCOCC1 ; CC(C)(C)OC(=O)N[C@@H](C(=O)N1CCC(OC2CCN(CC(=O)N3CCN(C(=O)c4cc(Cc5n[nH]c(=O)c6ccccc56)ccc4F)CC3)CC2)CC1)C1CCOCC1]

To a solution of intermediate 1-2 (20.67 mg, 79.73 μmol, 1 eq) in DMF (0.5 mL) was added HOAt (21.70 mg, 159.45 μmol, 22.31 μL, 2 eq), EDCI (76.42 mg, 398.63 μmol, 5 eq), NMM (80.64 mg, 797.26 mol, 87.65 μL, 10 eq), and intermediate 1-1 (50 mg, 79.73 μmol, 1 eq, HCl salt). The mixture was stirred at 25° C. for 1 hr. The reaction mixture was diluted with water (5 mL) and extracted with EA (5 mL*3). The combined organic layers were dried over Na₂SO₄, filtered and concentrated under reduced pressure to give a residue. The residue was purified by prep-HPLC (column: CD04-Welch Utimate C18 150*25*7 um; mobile phase: [water(FA)-ACN]; gradient: 10%-40% B over 8 min). Then the eluent was lyophilized to give product. I-849 (22 mg, 24.64 μmol, 30.90% yield, 98.319% purity, FA salt) was obtained as a white solid. LCMS (Method D): Rt=0.346 min, [M+H]⁺=832.5. ¹H NMR (400 MHz, METHANOL-d₄) δ=8.44 (s, 1H), 8.37 (d, J=7.2 Hz, 1H), 7.94 (d, J=3.6 Hz, 1H), 7.91-7.80 (m, 2H), 7.50 (s, 1H), 7.42-7.33 (m, 1H), 7.17 (t, J=8.8 Hz, 1H), 4.45-4.35 (m, 3H), 4.08-3.87 (m, 3H), 3.85-3.61 (m, 8H), 3.60-3.46 (m, 4H), 3.43-3.33 (m, 4H), 3.25-3.15 (m, 1H), 3.13-2.96 (m, 2H), 2.82-2.53 (m, 2H), 2.04-1.87 (m, 4H), 1.86-1.69 (m, 3H), 1.67-1.57 (m, 2H), 1.43 (s, 10H), 1.40-1.26 (m, 3H). ¹⁹F NMR (377 MHz, METHANOL-d₄), δ=−120.779 (s). SFC: Retention time: 1.536 min.

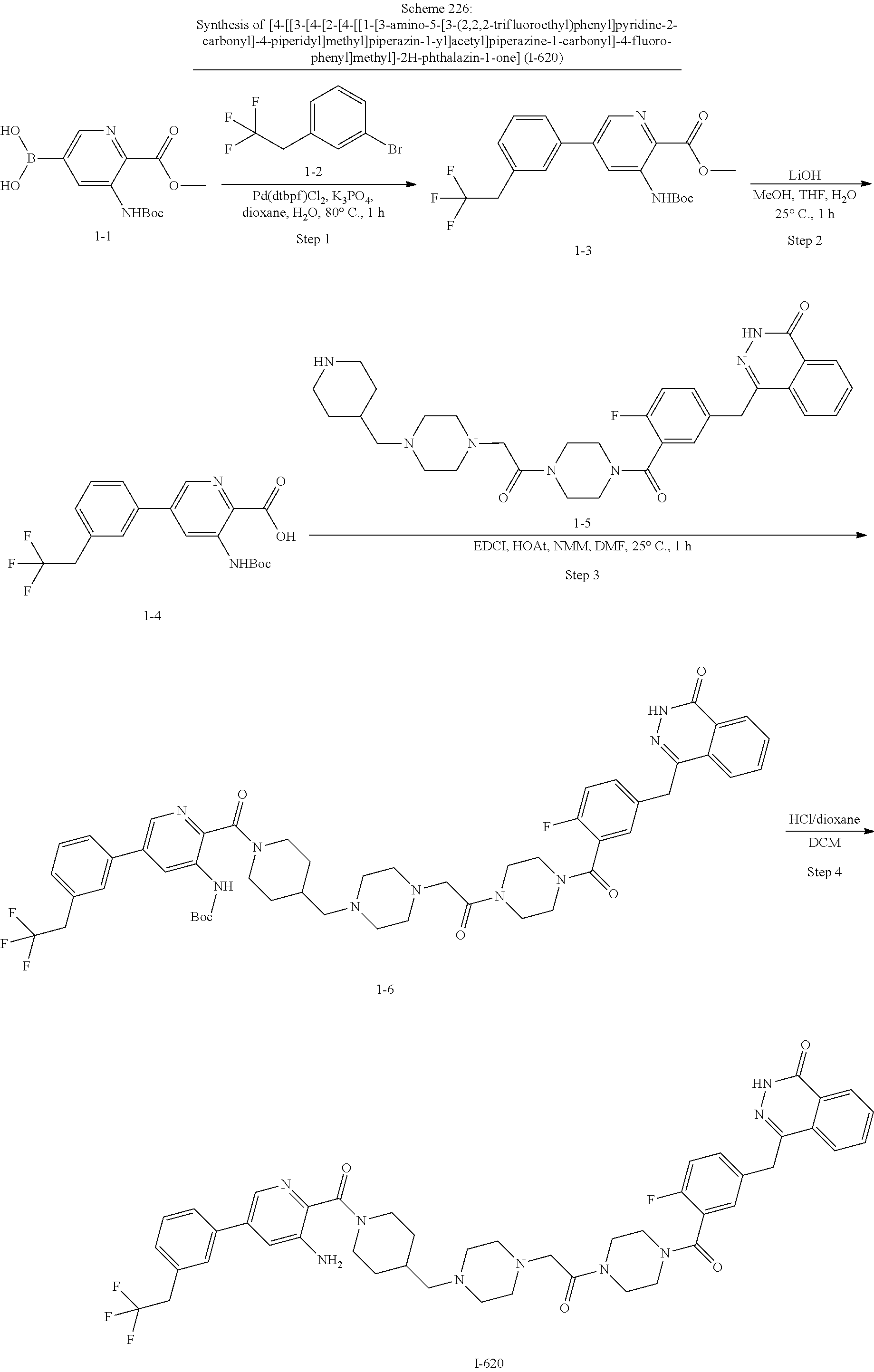

### Step 1: Synthesis of Intermediate 1-3.

To a mixture of intermediate 1-1 (180 mg, 607.94 μmol, 1 eq) in dioxane (1.5 mL) and H₂O (0.3 mL) was added intermediate 1-2 (145.32 mg, 607.94 μmol, 1 eq), K₃PO₄ (387.13 mg, 1.82 mmol, 3 eq) and Pd(dtbpf)Cl₂ (39.62 mg, 60.79 μmol, 0.1 eq) at 25° C., and the mixture was degassed and purged with N₂ 3 times, and then the mixture was stirred at 80° C. for 1 h under N₂ atmosphere. The reaction mixture was diluted with H₂O (3 mL) and extracted with EtOAc (3 mL×3), the combined organic layers were dried over Na₂SO₄, filtered and concentrated to give a residue. The residue was purified by column chromatography (SiO₂, PE/EA=1/0 to 6/4) and concentrated to give the product. Intermediate 1-3 (160 mg, 389.88 μmol, 64.13% yield) was obtained as a brown gum. LCMS (Method D): Retention time: 0.469 min, [M+H]⁺=411.1.

### Step 2: Synthesis of Intermediate 1-4.

To a mixture of intermediate 1-3 (130 mg, 316.77 μmol, 1 eq) in H₂O (0.5 mL), THF (0.5 mL) and MeOH (0.5 mL) was added LiOH·H₂O (39.88 mg, 950.32 μmol, 3 eq) at 25° C., the mixture was stirred at 25° C. for 1 h. The reaction mixture was diluted with H₂O (2 mL) and extracted with EA (2 mL×3), the combined organic layers were dried over Na₂SO₄, filtered and concentrated to give the crude product. The crude product was used for the next step directly. Intermediate 1-4 (110 mg, crude) was obtained as a yellow solid. LCMS (Method D): Retention time: 0.412 min, [M+H]⁺=397.1.

### Step 3: Synthesis of Intermediate 1-6.

To a solution of intermediate 1-4 (110 mg, 277.53 μmol, 1 eq) in DMF (2 mL) was added intermediate 1-5 (173.78 mg, 277.53 μmol, 1 eq, HCl salt), EDCI (159.61 mg, 832.58 μmol, 3 eq), HOAt (37.77 mg, 277.53 μmol, 38.82 μL, 1 eq) and NMM (140.35 mg, 1.39 mmol, 152.56 μL, 5 eq) at 25° C., the mixture was stirred at 25° C. for 1 h. The reaction mixture was concentrated to give a residue. The residue was purified by reversed phase HPLC (0.1% FA condition) and lyophilized to give the product. Intermediate 1-6 (50 mg, 51.65 μmol, 18.61% yield) was obtained as a yellow solid. LCMS (Method D): Retention time: 0.392 min, [M+H]⁺=968.3.

### Step 4: Synthesis of I-620

To a mixture of intermediate 1-6 (50 mg, 51.65 μmol, 1 eq) in DCM (0.5 mL) was added HCl/dioxane (2 M, 0.5 mL, 19.36 eq) at 25° C., and the mixture was stirred at 25° C. for 0.5 h. The reaction mixture was concentrated to give a residue. The residue was purified by reversed phase HPLC (0.1% FA condition) and lyophilized to give the product. I-620 (4.5 mg, 4.74 μmol, 9.17% yield, 96.236% purity, FA salt) was obtained as a white solid. LCMS (Method D): Retention time: 0.341 min, [M+H]⁺=868.3. ¹H NMR (400 MHz, METHANOL-d4) δ=8.37 (d, J=8.4 Hz, 1H), 8.09 (s, 1H), 7.99-7.92 (m, 1H), 7.91-7.80 (m, 2H), 7.65-7.58 (m, 2H), 7.53-7.47 (m, 2H), 7.45-7.35 (m, 3H), 7.17 (t, J=8.8 Hz, 1H), 4.73-4.55 (m, 2H), 4.39 (s, 2H), 3.84-3.65 (m, 6H), 3.64-3.49 (m, 5H), 3.25 (s, 1H), 2.95-2.86 (m, 1H), 2.70-2.44 (m, 9H), 2.37-2.27 (m, 2H), 2.08-1.99 (m, 1H), 1.97-1.86 (m, 2H), 1.80-1.71 (m, 1H), 0.93-0.86 (m, 1H). ¹⁹F NMR: (377 MHz, METHANOL-d₄) δ=−120.809 (s), −67.437 (s).

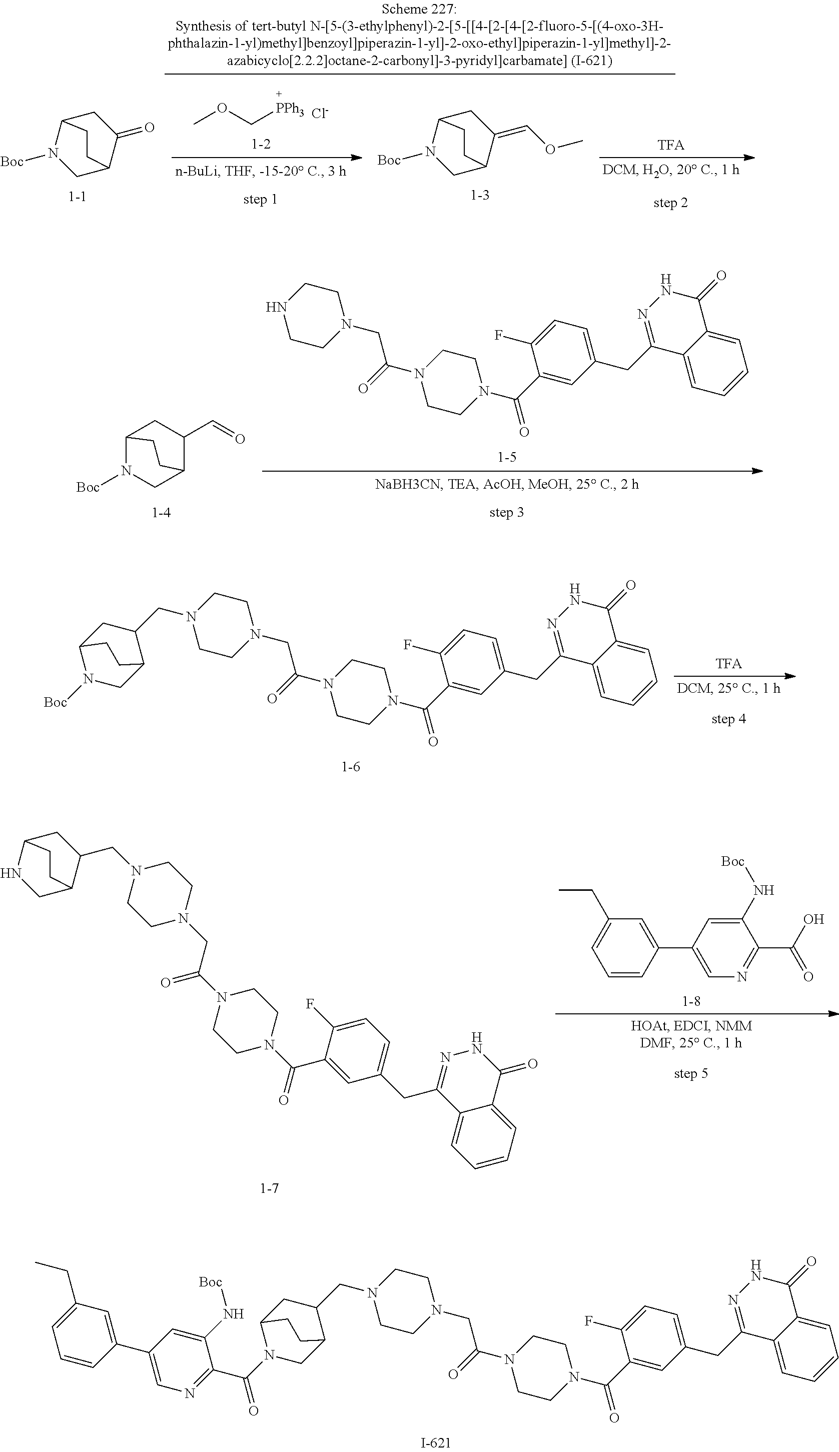

### Step 1: Synthesis of Intermediate 1-3

To a solution of intermediate 1-2 (2.28 g, 6.66 mmol, 1.5 eq) in THF (5 mL) was added n-BuLi (2.5 M, 2.66 mL, 1.5 eq) at −10° C. under N₂ atmosphere, and the mixture was stirred at 0° C. for 1 h. A solution of intermediate 1-1 (1 g, 4.44 mmol, 1 eq) in THF (5 mL) was added dropwise to the reaction solution at −20° C., and then the mixture was stirred for 2 h at 25° C. under N₂ atmosphere. The mixture was quenched with sat. NH₄Cl (20 mL) under N₂ atmosphere and extracted with EA (10 mL*3), dried over Na₂SO₄, filtered and concentrated under reduced pressure to give a residue. The crude product was purified by reversed phase HPLC (0.1% FA condition). The eluent was concentrated under reduced pressure to remove ACN, then lyophilized to afford product. Intermediate 1-3 (600 mg, 2.37 mmol, 53.36% yield) was obtained as a yellow oil. LCMS (Method D): Rt=0.438 min, [M-tBu+H]⁺=198.1. ¹H NMR (400 MHz, METHANOL-d4) δ=5.88 (s, 1H), 4.09 (br s, 1H), 3.55 (s, 1H), 3.52 (s, 2H), 3.30-3.23 (m, 1H), 3.01-2.89 (m, 1H), 2.50-2.22 (m, 2H), 1.97-1.82 (m, 1H), 1.78-1.57 (m, 3H), 1.45 (s, 10H).

### Step 2: Synthesis of Intermediate 1-4

To a solution of intermediate 1-3 (340 mg, 1.34 mmol, 1 eq) in DCM (2.5 mL) and H₂O (0.8 mL) was added TFA (918.17 mg, 8.05 mmol, 598.16 μL, 6 eq). The mixture was stirred at 25° C. for 2 hr. The reaction mixture was quenched by H₂O (5 mL), and then extracted with DCM (5 mL*3). The combined organic layers were dried over Na₂SO₄, filtered and concentrated under reduced pressure to give a residue. The crude product intermediate 1-4 (300 mg, 1.25 mmol, 93.41% yield) was obtained as a yellow oil.

### Step 3: Synthesis of Intermediate 1-6

To a solution of intermediate 1-5 (600 mg, 989.17 μmol, 1 eq, TFA salt) in MeOH (3 mL) was added TEA (600.56 mg, 5.94 mmol, 826.08 μL, 6 eq) and the mixture was stirred at 25° C. for 10 mins, and then AcOH (356.41 mg, 5.94 mmol, 339.76 μL, 6 eq) and intermediate 1-4 (236.72 mg, 989.17 μmol, 1 eq) were added into the mixture, and the mixture was stirred at 25° C. for 10 mins. Then NaBH₃CN (248.65 mg, 3.96 mmol, 4 eq) was added into the mixture. The mixture was stirred at 25° C. for 2 hrs. The residue was diluted with H₂O (5 mL) and extracted with EA (5 mL*3) dried over Na₂SO₄, filtered and concentrated under reduced pressure to give a residue. The residue was purified by flash silica gel chromatography (ISCO; 40 g SepaFlash Silica Flash Column, Eluent of 0˜100% Dichloromethane/Methanol@60 mL/min). The eluent was concentrated under reduced pressure to give product. Intermediate 1-6 (600 mg, 838.16 μmol, 84.73% yield) was obtained as a yellow oil. LCMS (Method D): Rt=0.336 min, [M+H]⁺=716.3. SFC: Retention time: 1.075 min, 1.187 min.

### Step 4: Synthesis of Intermediate 1-7

A solution of intermediate 1-6 (500 mg, 698.47 μmol, 1 eq) in TFA (1.5 mL) and DCM (3 mL) was stirred at 25° C. for 1 hr. The reaction mixture was concentrated under reduced pressure then washed by PE (3*1 mL) to give a residue. Intermediate 1-7 (500 mg, 685.15 μmol, 98.09% yield, TFA salt) was obtained as a colorless oil and used in the next step without further purification.

### Step 5: Synthesis of I-621

To a solution of intermediate 1-8 (100 mg, 292.07 μmol, 1 eq) in DMF (1 mL) was added EDCI (279.95 mg, 1.46 mmol, 5 eq), NMM (295.42 mg, 2.92 mmol, 321.10 μL, 10 eq) and HOAt (79.51 mg, 584.13 μmol, 81.71 μL, 2 eq), then intermediate 1-7 (234.45 mg, 321.27 μmol, 1.1 eq, TFA salt) was added into the mixture. The mixture was stirred at 25° C. for 2 hrs. The residue was diluted with H₂O (5 mL) and extracted with EA (5 mL*3), dried over Na₂SO₄, filtered and concentrated under reduced pressure to give a residue. The crude product was purified by reversed phase HPLC (0.1% FA condition). The eluent was concentrated under reduced pressure to remove ACN, then lyophilized to afford product. I-621 (50 mg, 47.04 μmol, 16.10% yield, 99.167% purity, TFA salt) was obtained as a white solid. LCMS (Method D): Rt=0.483 min, [M+H]⁺=940.5. ¹H NMR (400 MHz, METHANOL-d4) δ=8.59-8.39 (m, 2H), 8.37 (d, J=7.6 Hz, 1H), 8.00-7.90 (m, 1H), 7.90-7.78 (m, 2H), 7.55-7.45 (m, 3H), 7.42 (t, J=7.6 Hz, 1H), 7.36 (br d, J=6.8 Hz, 1H), 7.30 (br d, J=7.2 Hz, 1H), 7.17 (t, J=8.8 Hz, 1H), 4.39 (s, 2H), 3.89-3.62 (m, 6H), 3.58-3.33 (m, 5H), 2.81-2.71 (m, 4H), 2.67 (br d, J=8.8 Hz, 7H), 2.39-1.93 (m, 4H), 1.93-1.56 (m, 5H), 1.54 (s, 9H), 1.39-1.23 (m, 4H), 1.20 (s, 1H). ¹⁹F NMR: (377 MHz, METHANOL-d₄) δ=−120.794 (s), −76.954 (s). SFC: Retention time: 1.673 min, 1.716 min.

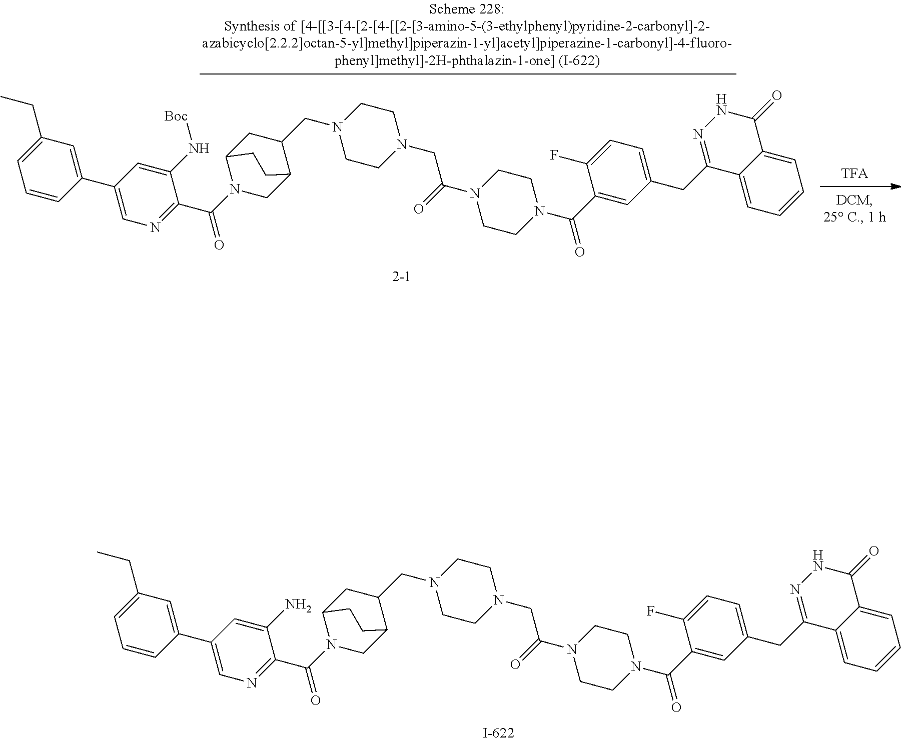 [SMILES: CCc1cccc(-c2cnc(C(=O)N3CC4CCC3CC4CN3CCN(CC(=O)N4CCN(C(=O)c5cc(Cc6n[nH]c(=O)c7ccccc67)ccc5F)CC4)CC3)c(NC(=O)OC(C)(C)C)c2)c1 ; CCc1cccc(-c2cnc(C(=O)N3CC4CCC3CC4CN3CCN(CC(=O)N4CCN(C(=O)c5cc(Cc6n[nH]c(=O)c7ccccc67)ccc5F)CC4)CC3)c(N)c2)c1]

To a solution of intermediate 2-1 (40 mg, 42.55 μmol, 1 eq) in DCM (0.3 mL) was added TFA (0.15 mL). The mixture was stirred at 25° C. for 2 hrs. The residue was concentrated under reduced pressure then washed by PE (3*1 mL) to give a residue. The crude product was purified by reversed phase HPLC (0.1% TFA condition), the eluent was concentrated under reduced pressure to remove ACN, then lyophilized to afford product. I-622 (20 mg, 20.61 μmol, 48.44% yield, 98.31% purity, TFA salt) was obtained as a white solid. LCMS (Method D): Rt=0.646 min, [M+H]⁺=840.4. ¹H NMR (400 MHz, METHANOL-d4) δ=8.37 (d, J=8.0 Hz, 1H), 8.20-8.09 (m, 1H), 8.05-7.92 (m, 1H), 7.91-7.79 (m, 2H), 7.59-7.49 (m, 2H), 7.48-7.40 (m, 3H), 7.39-7.33 (m, 1H), 7.30 (br d, J=7.6 Hz, 1H), 7.17 (t, J=8.8 Hz, 1H), 4.39 (s, 2H), 3.88-3.65 (m, 7H), 3.64-3.50 (m, 3H), 3.46-3.34 (m, 2H), 3.28-3.01 (m, 10H), 2.77-2.69 (m, 2H), 2.33 (d, J=10.4 Hz, 1H), 2.17-1.90 (m, 3H), 1.90-1.59 (m, 4H), 1.37-1.16 (m, 4H). ¹⁹F NMR: (377 MHz, METHANOL-d₄) δ=−120.664-−120.784 (m), −77.322 (s). SFC: Retention time: 1.931 min, 2.041 min.

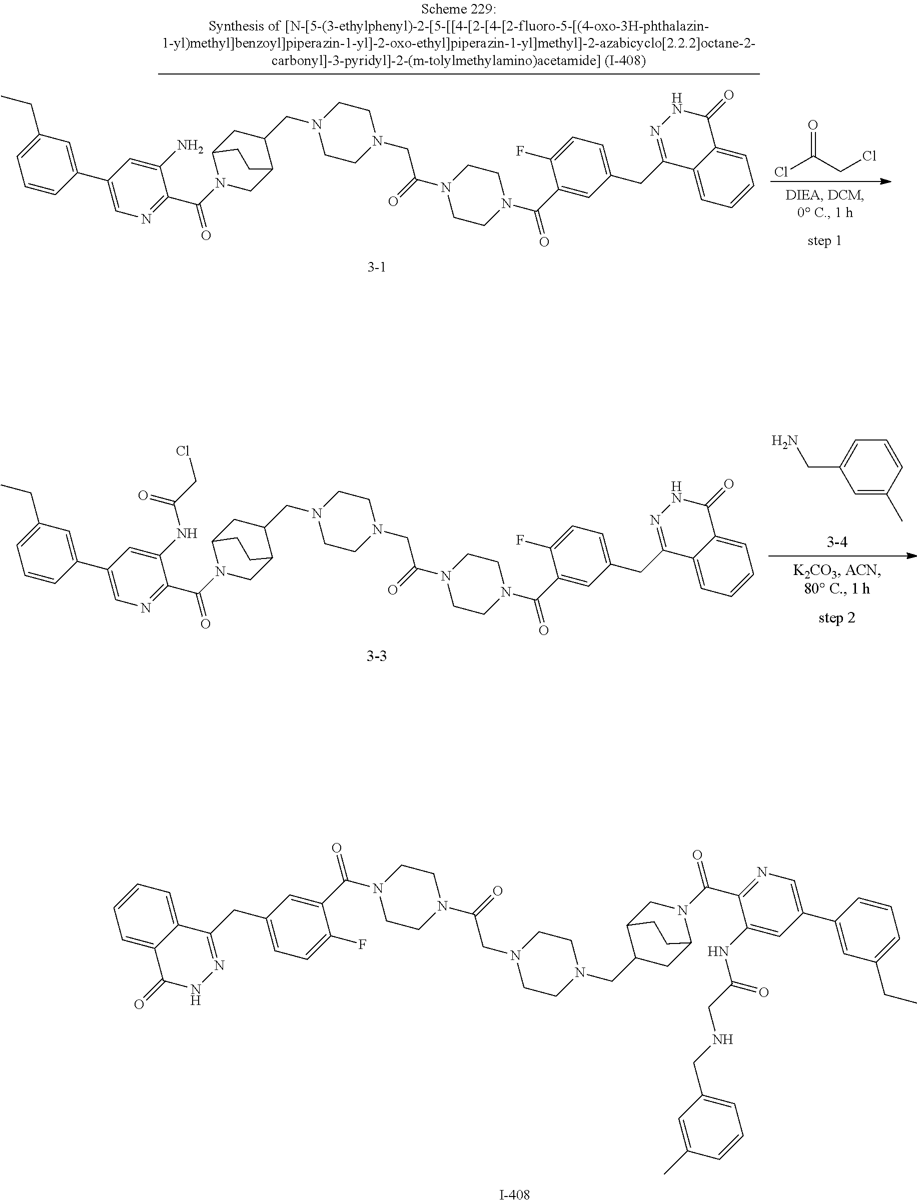

### Step 1: Synthesis of Intermediate 3-3

To a solution of intermediate 3-1 (150 mg, 157.23 μmol, 1 eq, TFA salt) in DCM (1.5 mL) was added DIEA (40.64 mg, 314.46 μmol, 54.77 μL, 2 eq) and intermediate 3-2 (17.76 mg, 157.23 μmol, 12.52 L, 1 eq) at 0° C. The mixture was stirred at 0° C. for 1 hr. The residue was diluted with H₂O (5 mL) and extracted with DCM (5 mL*3) dried over Na₂SO₄, filtered and concentrated under reduced pressure to give a residue. The residue was purified by flash silica gel chromatography (ISCO; 12 g SepaFlash Silica Flash Column, Eluent of 0˜100% Dichloromethane/Methanol@60 mL/min). The eluent was concentrated under reduced pressure to give product. Intermediate 3-3 (140 mg, 152.76 μmol, 97.16% yield) was obtained as yellow oil. LCMS (Method D): Rt=0.373 min, [M+H]⁺=916.3. SFC: Retention time: 2.136 min, 2.447 min

### Step 2: Synthesis of I-408

To a solution of intermediate 3-3 (100 mg, 109.11 μmol, 1 eq) and intermediate 3-4 (15.87 mg, 130.94 μmol, 16.43 μL, 1.2 eq) in ACN (1 mL) was added K₂CO₃ (30.16 mg, 218.23 μmol, 2 eq). The mixture was stirred at 40° C. for 2 hrs. The residue was diluted with H₂O (5 mL) and extracted with EA (5 mL*3), dried over Na₂SO₄, filtered and concentrated under reduced pressure to give a residue. The crude product was purified by reversed phase HPLC (0.1% FA condition), the eluent was concentrated under reduced pressure to remove ACN, then lyophilized to afford product. I-408 (15 mg, 14.65 μmol, 13.42% yield, 97.768% purity) was obtained as a white solid. LCMS: Rt=0.349 min, [M+H]⁺=1001.4. ¹H NMR (400 MHz, METHANOL-d4) δ=8.84-8.68 (m, 1H), 8.63-8.55 (m, 1H), 8.36 (br d, J=7.6 Hz, 1H), 7.94 (br s, 1H), 7.90-7.77 (m, 2H), 7.50 (br d, J=15.2 Hz, 3H), 7.45-7.34 (m, 2H), 7.30 (br d, J=6.4 Hz, 2H), 7.26-7.12 (m, 3H), 7.06 (br s, 1H), 4.68-4.45 (m, 1H), 4.38 (br s, 2H), 3.79 (br s, 3H), 3.76-3.60 (m, 5H), 3.59-3.45 (m, 3H), 3.42 (br s, 2H), 3.27-3.16 (m, 2H), 2.80-2.70 (m, 2H), 2.58-2.35 (m, 8H), 2.33 (br s, 4H), 2.24-1.96 (m, 4H), 1.92-1.66 (m, 3H), 1.66-1.40 (m, 2H), 1.39-1.09 (m, 4H). ¹⁹F NMR: (377 MHz, METHANOL-d₄) δ=−120.764 (s). SFC: Retention time: 0.901 min, 1.0241 min.

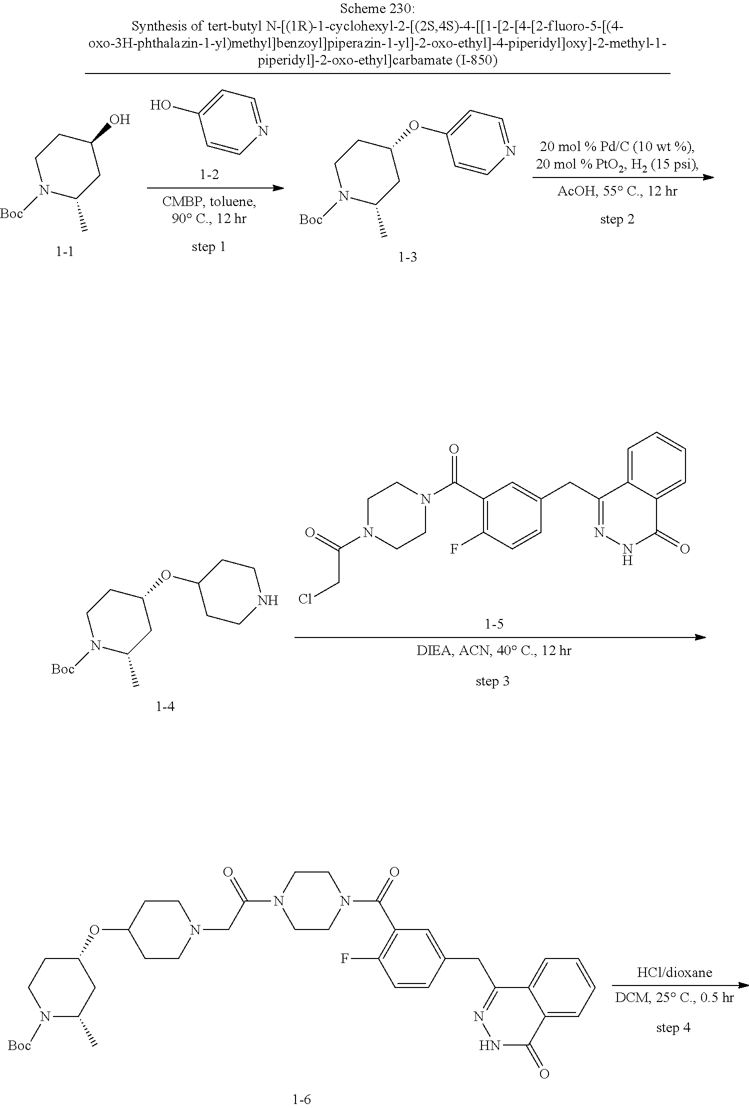 [SMILES: C[C@H]1C[C@H](O)CCN1C(=O)OC(C)(C)C ; Oc1ccncc1 ; C[C@H]1C[C@@H](Oc2ccncc2)CCN1C(=O)OC(C)(C)C ; C[C@H]1C[C@@H](OC2CCNCC2)CCN1C(=O)OC(C)(C)C ; O=C(CCl)N1CCN(C(=O)c2cc(Cc3n[nH]c(=O)c4ccccc34)ccc2F)CC1 ; C[C@H]1C[C@@H](OC2CCN(CC(=O)N3CCN(C(=O)c4cc(Cc5n[nH]c(=O)c6ccccc56)ccc4F)CC3)CC2)CCN1C(=O)OC(C)(C)C]

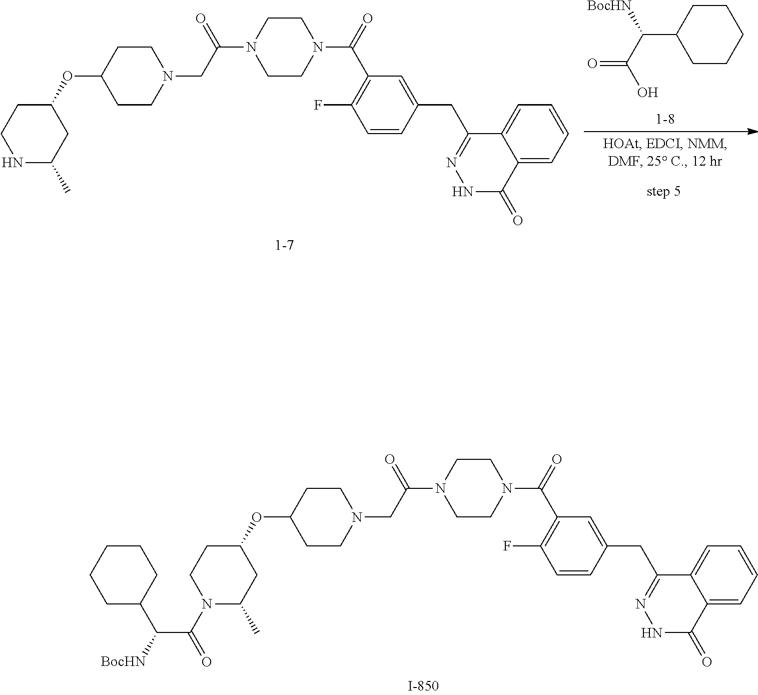 [SMILES: C[C@H]1C[C@@H](OC2CCN(CC(=O)N3CCN(C(=O)c4cc(Cc5n[nH]c(=O)c6ccccc56)ccc4F)CC3)CC2)CCN1 ; CC(C)(C)OC(=O)N[C@@H](C(=O)O)C1CCCCC1 ; C[C@H]1C[C@@H](OC2CCN(CC(=O)N3CCN(C(=O)c4cc(Cc5n[nH]c(=O)c6ccccc56)ccc4F)CC3)CC2)CCN1C(=O)[C@H](NC(=O)OC(C)(C)C)C1CCCCC1]

### Step 1: Synthesis of Intermediate 1-3

To a solution of intermediate 1-1 (100 mg, 464.49 μmol, 1 eq) and intermediate 1-2 (44.17 mg, 464.49 μmol, 1 eq) in toluene (1 mL) was added CMBP (224.21 mg, 928.99 μmol, 2 eq). The mixture was stirred at 90° C. for 2 hrs. The mixture was diluted with water (1 mL) and extracted with EA (2 mL*3). The combined organic layers were dried over sodium sulfate, filtered and concentrated under reduced pressure to give a residue. The residue was purified by reversed phase HPLC (0.1% FA condition), the eluent was concentrated under reduced pressure to remove ACN and lyophilized to give product. Intermediate 1-3 (60 mg, 203.17 μmol, 43.74% yield, 99% purity) was obtained as a brown solid. LCMS (Method D): Rt=0.289 min, [M+H]⁺=293.2. SFC: Rt=0.863 min. ¹H NMR (400 MHz, METHANOL-d4) δ=8.35 (d, J=6.4 Hz, 2H), 7.01 (d, J=6.4 Hz, 2H), 4.93-4.89 (m, 1H), 4.42-4.29 (m, 1H), 3.90-3.88 (m, 1H), 3.30-3.23 (m, 1H), 1.99 (s, 1H), 1.96 (s, 2H), 1.86-1.79 (m, 1H), 1.47 (s, 9H), 1.27 (d, J=7.2 Hz, 3H).

### Step 2: Synthesis of Intermediate 1-4

To a solution of intermediate 1-3 (100 mg, 342.03 μmol, 1 eq) in AcOH (2.5 mL) was added Pd/C (20 mg, 18.79 μmol, 10% purity, 5.49e−2 eq) and PtO₂ (30 mg, 132.11 μmol, 3.86e−1 eq) under N₂ atmosphere. The suspension was degassed and purged with H₂ 3 times. The mixture was stirred under H₂ (15 Psi) at 55° C. for 12 hrs. The resulting mixture was filtered, and the filtrate was concentrated under reduced pressure. The crude product was used in the next step without further purification. Intermediate 1-4 (80 mg, 260.04 μmol, 76.03% yield, 97% purity) was obtained as yellow oil. LCMS (Method D): Rt=0.303 min, [M+H]⁺=299.2. ¹H NMR (400 MHz, CHLOROFORM-d) δ=4.31-4.27 (m, 1H), 3.80-3.78 (m, 1H), 3.47-3.46 (m, 1H), 3.30-3.15 (m, 2H), 3.13-3.06 (m, 2H), 2.71-2.63 (m, 2H), 1.87-1.86 (m, 2H), 1.85-1.83 (m, 2H), 1.71-1.69 (m, 4H), 1.46 (s, 9H), 1.33 (d, J=7.2 Hz, 3H).

### Step 3: Synthesis of Intermediate 1-6

To a solution of intermediate 1-4 (80 mg, 268.08 μmol, 1 eq) and intermediate 1-5 (118.72 mg, 268.08 μmol, 1 eq) in ACN (1 mL) was added DIEA (103.94 mg, 804.24 μmol, 140.08 μL, 3 eq). The mixture was stirred at 40° C. for 12 hrs. The mixture was diluted with water (2 mL) and extracted with EA (3 mL*3). The combined organic layers were dried over sodium sulfate, filtered and concentrated under reduced pressure to give a residue. The residue was purified by flash silica gel chromatography (ISCO; 12 g SepaFlash Silica Flash Column, Eluent of 0˜17% MeOH/EA @40 mL/min) and then the eluent was concentrated in vacuo to give product. Intermediate 1-6 (70 mg, 93.36 μmol, 34.82% yield, 94% purity) was obtained as yellow oil. LCMS (Method D): Rt=0.361 min, [M+H]⁺=705.5. ¹H NMR (400 MHz, METHANOL-d4) δ=8.37 (d, J=7.6 Hz, 1H), 7.98-7.93 (m, 1H), 7.90-7.81 (m, 2H), 7.48 (d, J=2.8 Hz, 1H), 7.38 (s, 1H), 7.16 (t, J=8.8 Hz, 1H), 4.39 (s, 2H), 4.26 (s, 1H), 3.80-3.79 (m, 2H), 3.77-3.64 (m, 4H), 3.57-3.49 (m, 3H), 3.28 (s, 3H), 3.23-3.13 (m, 2H), 2.85-2.75 (m, 2H), 2.47-2.34 (m, 2H), 1.88 (s, 2H), 1.73 (s, 3H), 1.66-1.53 (m, 3H), 1.45 (s, 9H), 1.32-1.28 (m, 3H).

### Step 4: Synthesis of Intermediate 1-7

To a solution of intermediate 1-6 (60 mg, 85.13 μmol, 1 eq) in DCM (0.5 mL) was added HCl/dioxane (2 M, 0.5 mL). The mixture was stirred at 25° C. for 0.5 hr. The reaction mixture was concentrated to give intermediate 1-7 (60 mg, crude, HCl salt) as a yellow solid. LCMS (Method D): Rt=0.243 min, [M+H]⁺=605.4.

### Step 5: Synthesis of I-850

To a solution of intermediate 1-8 (24.08 mg, 93.58 μmol, 1 eq) in DMF (1 mL) was added EDCI (53.82 mg, 280.73 μmol, 3 eq), HOAt (12.74 mg, 93.58 μmol, 13.09 μL, 1 eq) and NMM (47.33 mg, 467.89 μmol, 51.44 μL, 5 eq). Then intermediate 1-7 (60 mg, 93.58 μmol, 1 eq, HCl salt) was added into the mixture. The mixture was stirred at 25° C. for 1 hr. The mixture was diluted with water (1 mL) and extracted with EA (2 mL*3). The combined organic layers were dried over sodium sulfate, filtered and concentrated under reduced pressure to give a residue. The residue was purified by reversed phase HPLC (01% FA condition), the eluent was concentrated under reduced pressure to remove ACN and lyophilized to give product. I-850 (20.5 mg, 23.03 μmol, 24.61% yield, FA salt) was obtained as a yellow solid. LCMS: Rt=0.407 min, [M+H]⁺=844.4. SFC: Rt=2.040 min. ¹H NMR (400 MHz, METHANOL-d4) δ=8.50 (s, 1H), 8.37 (d, J=8.0 Hz, 1H), 7.98-7.92 (m, 1H), 7.91-7.81 (m, 2H), 7.49 (d, J=2.8 Hz, 1H), 7.38-7.37 (m, 1H), 7.17-7.16 (m, 1H), 6.54-6.39 (m, 1H), 4.77 (d, J=1.6 Hz, 1H), 4.39 (s, 2H), 4.34-4.25 (m, 1H), 3.84 (s, 1H), 3.83-3.72 (m, 3H), 3.69 (s, 2H), 3.61-3.50 (m, 4H), 3.47-3.45 (m, 1H), 3.34 (s, 2H), 3.17-3.05 (m, 1H), 2.91-2.80 (m, 2H), 2.58-2.41 (m, 2H), 1.94-1.84 (m, 3H), 1.73 (s, 6H), 1.67 (d, J=10.4 Hz, 5H), 1.49-1.46 (m, 1H), 1.43 (s, 9H), 1.30 (d, J=4.8 Hz, 2H), 1.26-1.18 (m, 3H), 1.10-1.00 (m, 2H).

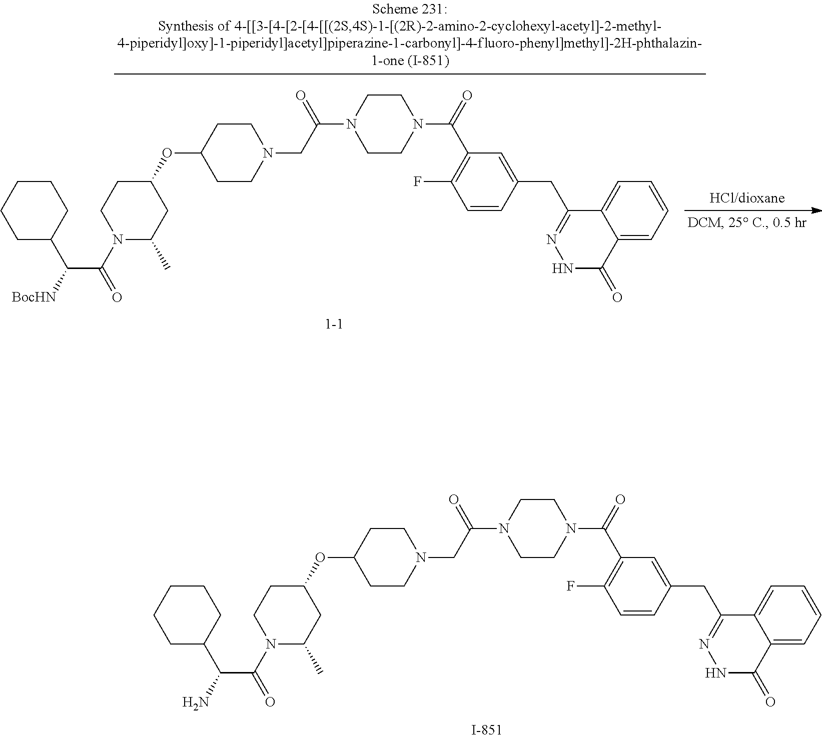 [SMILES: C[C@H]1C[C@@H](OC2CCN(CC(=O)N3CCN(C(=O)c4cc(Cc5n[nH]c(=O)c6ccccc56)ccc4F)CC3)CC2)CCN1C(=O)[C@H](NC(=O)OC(C)(C)C)C1CCCCC1 ; C[C@H]1C[C@@H](OC2CCN(CC(=O)N3CCN(C(=O)c4cc(Cc5n[nH]c(=O)c6ccccc56)ccc4F)CC3)CC2)CCN1C(=O)[C@H](N)C1CCCCC1]

To a solution of intermediate 1-1 (30 mg, 35.54 μmol, 1 eq) in DCM (0.3 mL) was added HCl/dioxane (2 M, 0.3 mL). The mixture was stirred at 25° C. for 0.5 hr. The reaction mixture was concentrated to give a residue The residue was purified by prep-HPLC (column: CD02-Waters Xbidge BEH C18 150*25*10 um; mobile phase: [water (HCl)-ACN]; gradient: 5%-35% B over 10 min) and the eluent was concentrated under reduced pressure to remove ACN and lyophilized to give product. I-851 (20.44 mg, 26.00 μmol, 73.16% yield, 99.281% purity, HCl salt) was obtained as a white solid. LCMS (Method D): Rt=0.296 min, [M+H]⁺=744.6. SFC: Rt=2.820 min. ¹H NMR (400 MHz, METHANOL-d4) δ=8.37 (d, J=7.6 Hz, 1H), 7.98-7.94 (m, 1H), 7.92-7.87 (m, 1H), 7.86-7.82 (m, 1H), 7.54-7.49 (m, 1H), 7.38-7.36 (m, 1H), 7.18-7.17 (m, 1H), 4.84 (s, 1H), 4.39 (s, 2H), 4.35 (d, J=6.0 Hz, 1H), 4.33-4.27 (m, 2H), 4.17 (d, J=4.8 Hz, 1H), 3.98-3.88 (m, 2H), 3.83 (d, J=5.2 Hz, 1H), 3.78 (d, J=4.8 Hz, 1H), 3.73 (d, J=4.0 Hz, 1H), 3.68-3.65 (m, 1H), 3.57-3.52 (m, 2H), 3.49-3.46 (m, 1H), 3.39 (s, 2H), 3.36-3.33 (m, 2H), 3.19 (d, J=11.6 Hz, 1H), 2.31-2.17 (m, 1H), 2.09 (s, 3H), 1.94 (d, J=15.6 Hz, 2H), 1.88-1.78 (m, 6H), 1.73 (d, J=10.8 Hz, 3H), 1.51-1.46 (m, 1H), 1.38-1.32 (m, 3H), 1.26-1.12 (m, 4H). ¹⁹F NMR (376 MHz, MeOD-d4) δ=−120.764.

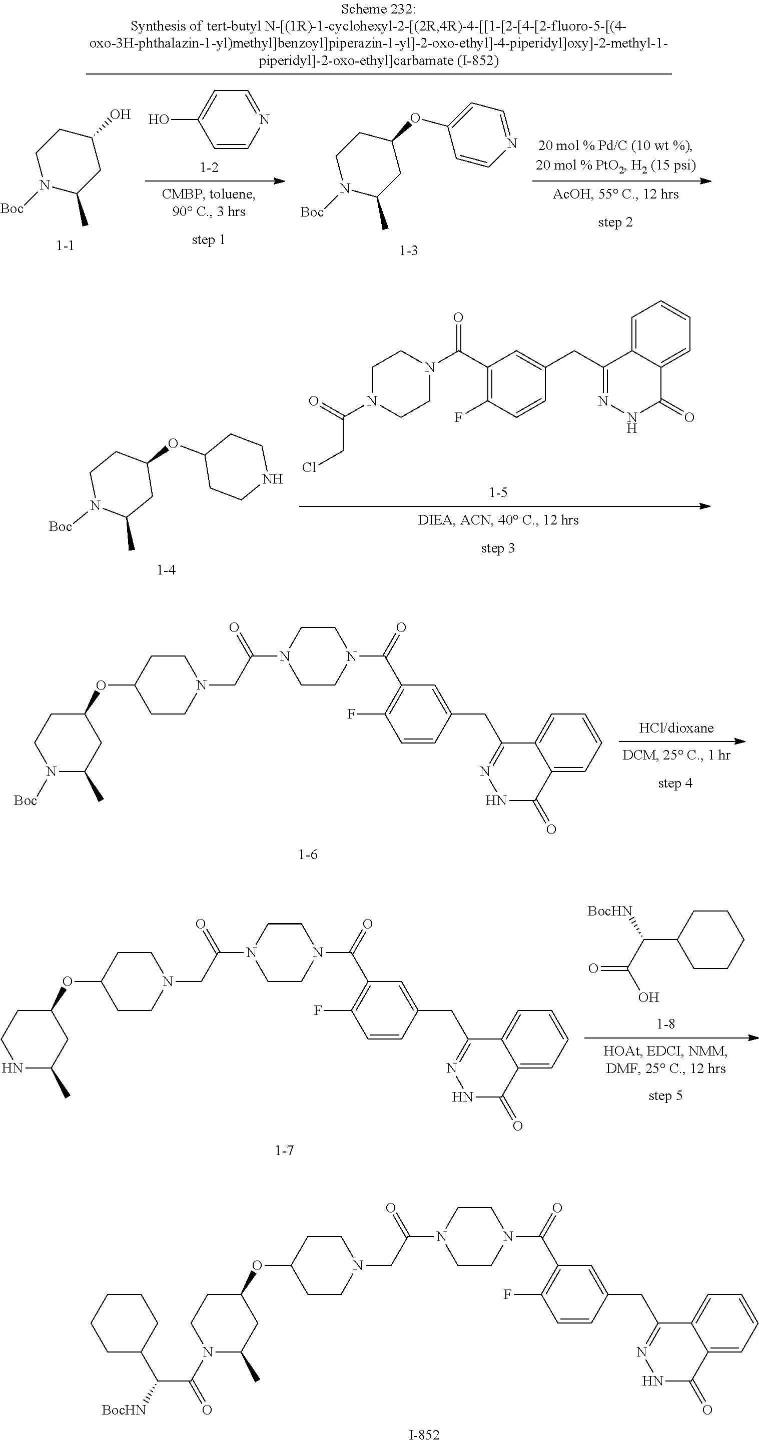

### Step 1: Synthesis of Intermediate 1-3.

To a solution of intermediate 1-1 (1 g, 4.64 mmol, 1 eq) in toluene (10 mL) was added intermediate 1-2 (441.73 mg, 4.64 mmol, 1 eq) and CMBP (2.24 g, 9.29 mmol, 2 eq). The mixture was stirred at 90° C. for 3 hrs under N₂ atmosphere. The reaction mixture was diluted with H₂O (5 mL) and extracted with EA (5 mL*2). The combined organic layers were dried over Na₂SO₄, filtered and concentrated under reduced pressure to give a residue. The residue was purified by flash silica gel chromatography (ISCO; 40 g SepaFlash Silica Flash Column, Eluent of 0˜60% Ethyl acetate/Petroleum ethergradient @40 mL/min). The eluent was concentrated under reduced pressure to give intermediate 1-3 (840 mg, 2.85 mmol, 61.31% yield, 99.115% purity) as a yellow solid. LCMS (Method D): Retention time: 0.286 min, [M+H]⁺=293.1. SFC: Retention time: 3.944 min. ¹H NMR (400 MHz, METHANOL-d₄) δ=8.42-8.29 (m, 2H), 7.05-6.96 (m, 2H), 4.93-4.88 (m, 1H), 4.41-4.31 (m, 1H), 3.94-3.86 (m, 1H), 3.30-3.23 (m, 1H), 2.01-1.94 (m, 3H), 1.84-1.77 (m, 1H), 1.47 (s, 9H), 1.27 (d, J=7.2 Hz, 3H).

### Step 2: Synthesis of Intermediate 1-4.

To a solution of Pd/C (400 mg, 10% purity) and PtO₂ (400 mg) in AcOH (10 mL) was added intermediate 1-3 (800 mg, 2.74 mmol, 1 eq) under N₂ atmosphere. The suspension was degassed and purged with H₂ 3 times. The mixture was stirred under H₂ (15 Psi) at 55° C. for 12 hrs. The mixture was filtered and the filter cake was washed with MeOH (3 mL*3), then the filtrate was concentrated under reduced pressure to give a residue. The mixture was diluted with H₂O (8 mL) and NH₃·H₂O was added to adjust pH to 8. The mixture was extracted with DCM (5 mL*2). The combined organic layers were dried over Na₂SO₄, filtered and concentrated under reduced pressure to give intermediate 1-4 (750 mg, 2.46 mmol, 89.88% yield, 97.856% purity) as a colorless oil. LCMS (Method D): Retention time: 0.299 min, [M+H]⁺=299.2. ¹H NMR (400 MHz, METHANOL-d₄) δ=4.37-4.17 (m, 1H), 3.88-3.65 (m, 2H), 3.28-3.14 (m, 2H), 3.11-2.86 (m, 2H), 1.99-1.91 (m, 1H), 1.82-1.72 (m, 4H), 1.72-1.47 (m, 5H), 1.45 (s, 9H), 1.33-1.28 (m, 3H).

### Step 3: Synthesis of Intermediate 1-6.

To a solution of intermediate 1-4 (750 mg, 2.51 mmol, 1 eq) in ACN (10 mL) was added intermediate 1-5 (890.43 mg, 2.01 mmol, 0.8 eq) and DIEA (974.45 mg, 7.54 mmol, 1.31 mL, 3 eq). The mixture was stirred at 40° C. for 12 hrs. The reaction mixture was diluted with H₂O (10 mL) and extracted with DCM (5 mL*2). The combined organic layers were dried over Na₂SO₄, filtered and concentrated under reduced pressure to give a residue. The crude product was purified by flash silica gel chromatography (ISCO; 80 g SepaFlash Silica Flash Column, Eluent of 0-60% Ethyl acetate/Methanol @60 mL/min). The eluent was concentrated under reduced pressure to give intermediate 1-6 (1 g, 1.39 mmol, 55.44% yield, 98.201% purity) was obtained as a yellow solid. LCMS (Method D): Retention time: 0.366 min, [M+H]⁺=705.2. SFC: Retention time: 1.989 min. ¹H NMR (400 MHz, METHANOL-d₄) δ=8.41-8.33 (m, 1H), 7.99-7.92 (m, 1H), 7.91-7.81 (m, 2H), 7.52-7.45 (m, 1H), 7.41-7.34 (m, 1H), 7.21-7.13 (m, 1H), 4.64-4.55 (m, 1H), 4.39 (s, 2H), 4.30-4.21 (m, 1H), 3.83-3.65 (m, 6H), 3.59-3.46 (m, 3H), 3.37-3.33 (m, 1H), 3.27-3.24 (m, 1H), 3.22-3.14 (m, 2H), 2.80-2.67 (m, 2H), 2.38-2.24 (m, 2H), 1.92-1.80 (m, 2H), 1.79-1.70 (m, 3H), 1.65-1.51 (m, 3H), 1.45 (s, 9H), 1.31-1.28 (m, 3H). ¹H NMR (400 MHz, METHANOL-d₄) δ=−120.779.

### Step 4: Synthesis of Intermediate 1-7.

A solution of intermediate 1-6 (100 mg, 141.88 μmol, 1 eq) in DCM (0.4 mL) and HCl/dioxane (2 M, 1 mL) was stirred at 25° C. for 1 hr. The mixture was concentrated in vacuum to give intermediate 1-7 (95 mg, crude, HCl salt) as a yellow solid. LCMS (Method D): Retention time: 0.236 min, [M+H]⁺=605.2.

### Step 5: Synthesis of I-852.

To a solution of intermediate 1-7 (90 mg, 140.37 μmol, 1 eq, HCl salt) in DMF (1 mL) was added intermediate 1-8 (36.12 mg, 140.37 μmol, 1 eq), EDCI (80.73 mg, 421.10 μmol, 3 eq), HOAt (19.11 mg, 140.37 μmol, 19.64 μL, 1 eq) and NMM (70.99 mg, 701.84 μmol, 77.16 μL, 5 eq). The mixture was stirred at 25° C. for 12 hrs. The reaction mixture was diluted with H₂O (1 mL) and extracted with EA (2 mL*2). The combined organic layers were washed with brine (2 mL*1), dried over Na₂SO₄, filtered and concentrated under reduced pressure to give a residue. The crude product was purified by reversed-phase HPLC (0.10% FA condition). The eluent was concentrated under reduced pressure to remove ACN and then lyophilized to give I-852 (28.73 mg, 31.98 μmol, 22.79% yield, 99.084% purity, FA salt) as a white solid. LCMS (Method D): Retention time: 0.406 min, [M+H]⁺=844.4. SFC Data: Retention time: 2.031 min. ¹H NMR (400 MHz, METHANOL-d₄) δ=8.59-8.43 (m, 1H), 8.37 (d, J=7.6 Hz, 1H), 8.00-7.92 (m, 1H), 7.91-7.80 (m, 2H), 7.54-7.46 (m, 1H), 7.41-7.35 (m, 1H), 7.22-7.13 (m, 1H), 6.72-6.46 (m, 1H), 4.72-4.49 (m, 1H), 4.39 (s, 2H), 4.33-4.22 (m, 1H), 3.92-3.71 (m, 4H), 3.70-3.59 (m, 4H), 3.56-3.47 (m, 3H), 3.39-3.32 (m, 2H), 3.22-2.86 (m, 3H), 2.78-2.53 (m, 2H), 1.98-1.89 (m, 2H), 1.85-1.53 (m, 12H), 1.51-1.47 (m, 1H), 1.43 (s, 9H), 1.31 (s, 2H), 1.28-1.14 (m, 3H), 1.12-0.92 (m, 2H). ¹⁹F NMR (376 MHz, MeOD-d6) δ=−120.779.

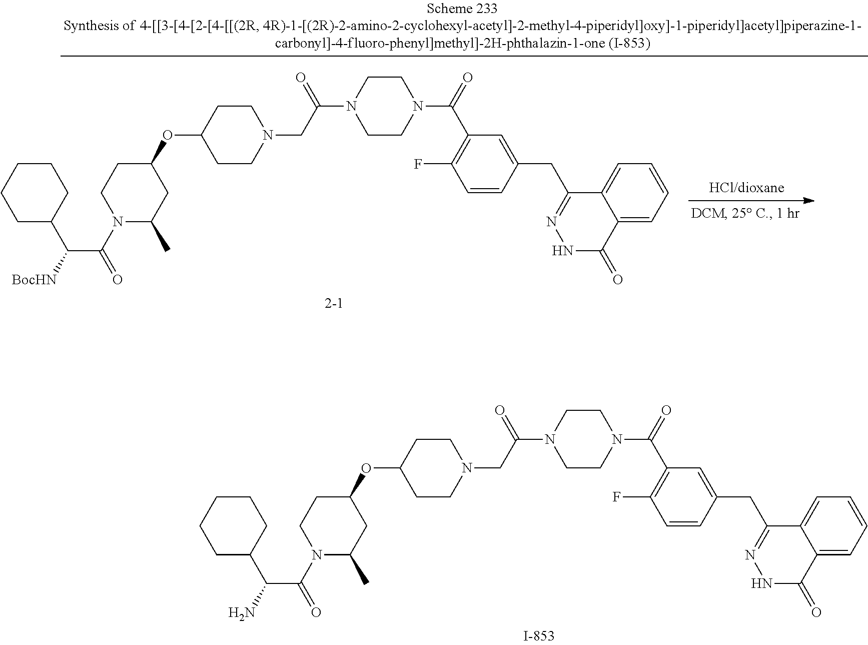 [SMILES: C[C@@H]1C[C@H](OC2CCN(CC(=O)N3CCN(C(=O)c4cc(Cc5n[nH]c(=O)c6ccccc56)ccc4F)CC3)CC2)CCN1C(=O)[C@H](NC(=O)OC(C)(C)C)C1CCCCC1 ; C[C@@H]1C[C@H](OC2CCN(CC(=O)N3CCN(C(=O)c4cc(Cc5n[nH]c(=O)c6ccccc56)ccc4F)CC3)CC2)CCN1C(=O)[C@H](N)C1CCCCC1]

A solution of intermediate 2-1 (100 mg, 118.48 μmol, 1 eq) in DCM (0.4 mL) and HCl/dioxane (2 M, 1.00 mL) was stirred at 25° C. for 1 hr. The mixture was concentrated in vacuum. The crude product was purified by prep-HPLC (column: CD02-Waters Xbidge BEH C18 150*25*10 um; mobile phase: [water (HCl)-ACN]; gradient: 3%-33% B over 10 min). The eluent was concentrated under reduced pressure to remove ACN and then lyophilized to give I-853 (36.86 mg, 46.66 μmol, 39.38% yield, 98.786% purity, HCl salt) as a white solid. LCMS (Method D): Retention time: 0.298 min, (M+H)=744.4. SFC: Retention time: 3.642 min. ¹H NMR (400 MHz, METHANOL-d₄) δ=8.37 (d, J=7.6 Hz, 1H), 7.99-7.94 (m, 1H), 7.91-7.81 (m, 2H), 7.54-7.48 (m, 1H), 7.41-7.35 (m, 1H), 7.20-7.14 (m, 1H), 4.75-4.65 (m, 1H), 4.39 (s, 2H), 4.38-4.34 (m, 1H), 4.33-4.27 (m, 2H), 4.00-3.88 (m, 2H), 3.86-3.81 (m, 1H), 3.80-3.77 (m, 1H), 3.75-3.71 (m, 1H), 3.70-3.60 (m, 2H), 3.57-3.52 (m, 2H), 3.49-3.45 (m, 1H), 3.41-3.38 (m, 2H), 3.37-3.33 (m, 2H), 3.22-3.13 (m, 1H), 2.36-2.15 (m, 1H), 2.08 (s, 3H), 1.99-1.91 (m, 1H), 1.89-1.78 (m, 6H), 1.76-1.62 (m, 4H), 1.59-1.48 (m, 1H), 1.39-1.31 (m, 3H), 1.27-1.14 (m, 4H). ¹⁹F NMR (376 MHz, MeOD-d₆) δ=−120.757.

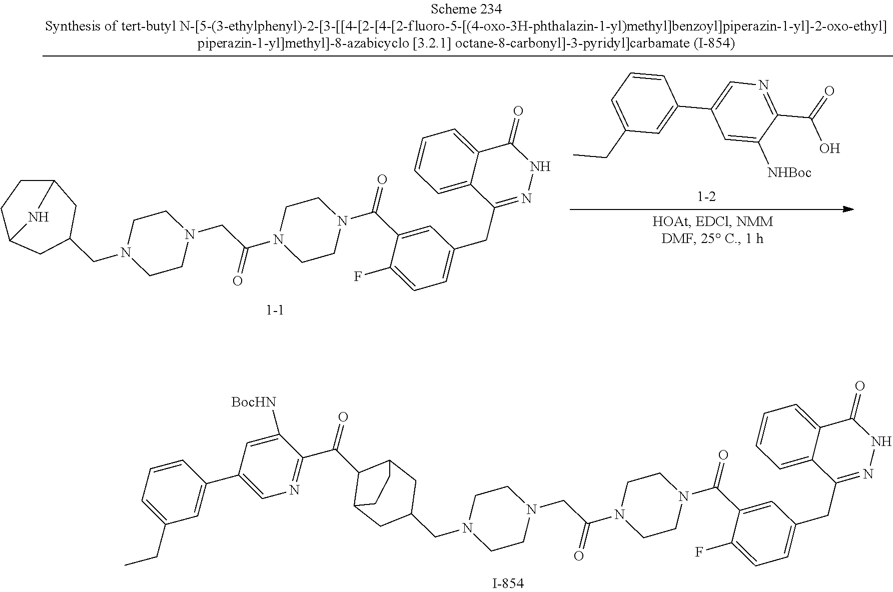

To a solution of intermediate 1-2 (55.61 mg, 162.41 μmol, 1 eq) and intermediate 1-1 (100 mg, 162.41 μmol, 1 eq) in DMF (2 mL) was added NMM (82.13 mg, 812.03 μmol, 89.28 μL, 5 eq), EDCI (155.67 mg, 812.03 μmol, 5 eq) and HOAt (44.21 mg, 324.81 μmol, 45.44 μL, 2 eq). The mixture was stirred at 25° C. for 1 hr. The reaction mixture was diluted with water (2 mL) and extracted with DCM (2 mL*3), the combined organic phase was dried by Na₂SO₄, and concentrated under reduced pressure to give a residue. The residue was purified by reversed-phase column (0.1% FA condition) and the eluent was lyophilized to give product. I-854 (35 mg, 33.29 μmol, 20.50% yield, 93.789% purity, FA salt) was obtained as a yellow solid. LCMS (Method D): Retention time: 0.437 min, [M+H]⁺=940.2. ¹H NMR (400 MHz, METHANOL-d4) δ=8.63-8.51 (m, 2H), 8.49 (s, 1H), 8.37 (d, J=7.6 Hz, 1H), 8.01-7.91 (m, 1H), 7.91-7.77 (m, 2H), 7.54-7.46 (m, 3H), 7.45-7.35 (m, 2H), 7.31 (d, J=7.6 Hz, 1H), 7.16 (t, J=9.2 Hz, 1H), 4.55-4.35 (m, 3H), 3.85-3.63 (m, 5H), 3.58-3.35 (m, 5H), 3.27 (s, 1H), 2.79-2.70 (m, 3H), 2.68-2.51 (m, 7H), 2.33 (d, J=14.4 Hz, 3H), 2.12-2.02 (m, 2H), 1.84 (s, 3H), 1.71 (s, 2H), 1.54 (s, 10H), 1.29 (t, J=7.6 Hz, 3H). ¹⁹F NMR: (377 MHz, METHANOL-d₄) δ=−120.792 (s).

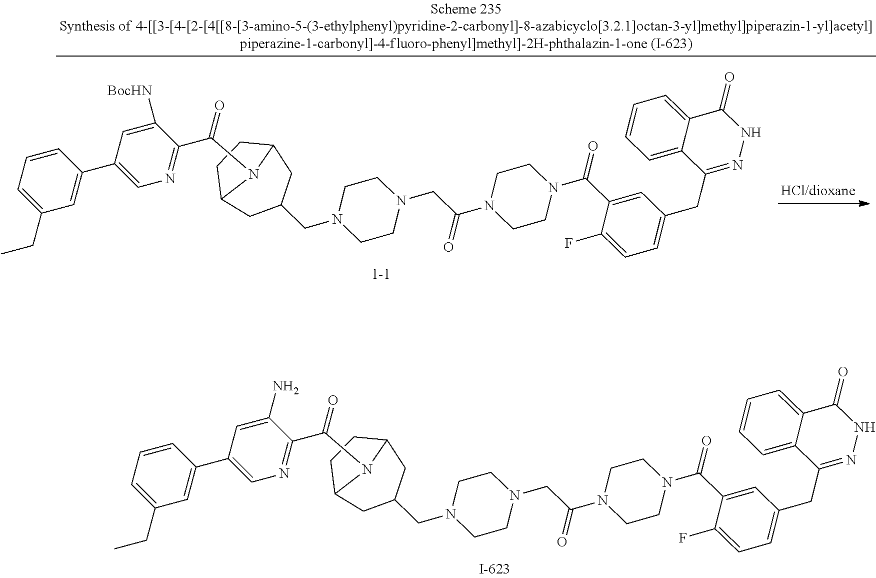 [SMILES: CCc1cccc(-c2cnc(C(=O)N3C4CCC3CC(CN3CCN(CC(=O)N5CCN(C(=O)c6cc(Cc7n[nH]c(=O)c8ccccc78)ccc6F)CC5)CC3)C4)c(NC(=O)OC(C)(C)C)c2)c1 ; CCc1cccc(-c2cnc(C(=O)N3C4CCC3CC(CN3CCN(CC(=O)N5CCN(C(=O)c6cc(Cc7n[nH]c(=O)c8ccccc78)ccc6F)CC5)CC3)C4)c(N)c2)c1]

A mixture of intermediate 1-1 (25 mg, 26.59 μmol, 1 eq) in HCl/dioxane (2 M, 0.3 mL) was stirred at 25° C. for 1 hr. The mixture was concentrated under reduced pressure to give a residue. The residue was lyophilized to give a product. I-623 (18 mg, 19.45 μmol, 73.12% yield, 94.682% purity, HCl salt) was obtained as a blue solid. LCMS (Method D): Retention time: 0.373 min, [M+H]⁺=840.4. ¹H NMR (400 MHz, METHANOL-d4) δ=8.37 (d, J=7.2 Hz, 1H), 8.33 (d, J=1.6 Hz, 1H), 8.02-7.92 (m, 2H), 7.91-7.81 (m, 2H), 7.57 (s, 1H), 7.55-7.49 (m, 2H), 7.48-7.44 (m, 1H), 7.41-7.35 (m, 2H), 7.17 (t, J=9.2 Hz, 1H), 4.39 (s, 2H), 4.32 (d, J=12.8 Hz, 2H), 3.84 (s, 1H), 3.78 (d, J=4.0 Hz, 1H), 3.71 (d, J=1.6 Hz, 1H), 3.66 (s, 6H), 3.56 (s, 3H), 3.41 (s, 2H), 3.14 (d, J=1.2 Hz, 3H), 2.94-2.87 (m, 1H), 2.79-2.72 (m, 2H), 2.64-2.56 (m, 1H), 2.22-2.15 (m, 2H), 2.02-1.92 (m, 4H), 1.74-1.61 (m, 2H), 1.56 (s, 1H), 1.32-1.27 (m, 4H). ¹⁹F NMR (376 MHz, MeOD-d4) δ=−120.710.

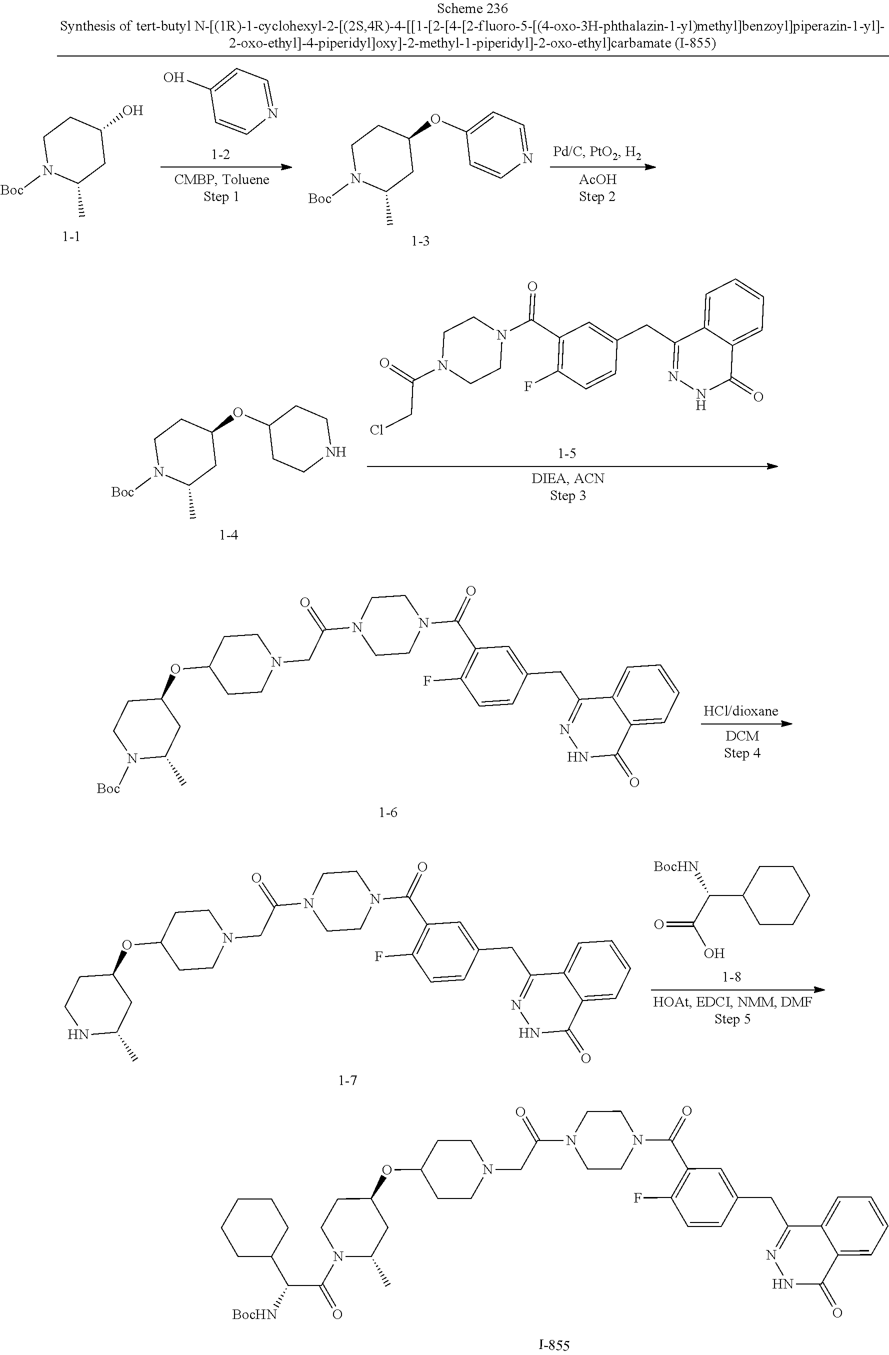

### Step 1: Synthesis of Intermediate 1-3.

To a mixture of intermediate 1-1 (1.6 g, 7.43 mmol, 1 eq) and intermediate 1-2 (706.77 mg, 7.43 mmol, 1 eq) in toluene (16 mL) was added CMBP (3.59 g, 14.86 mmol, 2 eq), then the mixture was stirred at 90° C. for 16 hours. The reaction mixture was concentrated under reduced pressure to give a residue. The crude product was purified by reversed-phase HPLC (0.1% FA condition). The eluent was concentrated under reduced pressure to remove ACN and then lyophilized to give intermediate 1-3 (700 mg, 2.39 mmol, 32.22% yield) as a white solid. LCMS (Method D): Retention time=0.292 min, [M+H]⁺=293.1. SFC: Retention time=0.760 min. ¹H NMR (400 MHz, METHANOL-d₄) δ=8.48-8.40 (m, 2H), 6.87-6.81 (m, 2H), 4.71-4.56 (m, 2H), 4.18-4.13 (m, 1H), 3.10-2.98 (m, 1H), 2.17-2.09 (m, 1H), 2.03-1.97 (m, 1H), 1.77 (d, J=5.6 Hz, 1H), 1.48 (s, 9H), 1.26 (d, J=7.2 Hz, 3H).

### Step 2: Synthesis of Intermediate 1-4.

To a mixture of intermediate 1-3 (500 mg, 1.71 mmol, 1 eq) in AcOH (10 mL) was added PtO₂ (300.18 mg, 1.32 mmol, 7.73e−1 eq) and Pd/C (200.00 mg, 187.93 μmol, 10% purity, 0.11 eq). The suspension was degassed and purged with N₂ 3 times, and then the mixture was degassed and purged with H₂ 3 times, then the mixture was stirred at 55° C. for 16 hours under H₂ atmosphere (15 psi). The reaction mixture was filtered and concentrated under reduced pressure to give intermediate 1-4 (450 mg, 1.51 mmol, 88.18% yield) as a yellow oil. LCMS (Method D): Retention time: 0.291 min, [M+H]⁺=299.2.

### Step 3: Synthesis of Intermediate 1-6.

To a mixture of intermediate 1-4 (400 mg, 1.34 mmol, 1 eq) and intermediate 1-5 (415.53 mg, 938.28 μmol, 0.7 eq) in ACN (5 mL) was added DIEA (519.71 mg, 4.02 mmol, 700.41 μL, 3 eq), then the mixture was stirred at 40° C. for 16 hours. The reaction mixture was concentrated under reduced pressure to give a residue. The residue was purified by flash silica gel chromatography (ISCO; 40 g SepaFlash Silica Flash Column, Eluent of 0˜10% Ethyl acetate/MeOH gradient @80 mL/min). The eluent was concentrated under reduced pressure to give intermediate 1-6 (200 mg, crude) as a white solid. LCMS (Method D): Retention time: 0.346 min, (M+H)=705.7.

### Step 4: Synthesis of Intermediate 1-7.

To a mixture of intermediate 1-6 (30 mg, 42.56 μmol, 1 eq) in DCM (0.5 mL) was added HCl/dioxane (2 M, 0.5 mL, 23.49 eq) and the mixture was stirred at 20° C. for 10 mins. The reaction mixture was concentrated under reduced pressure to give intermediate 1-7 (25 mg, 38.99 μmol, 91.61% yield, HCl salt) as a white solid. LCMS (Method D): Retention time: 0.234 min, (M+H)=605.2.

### Step 5: Synthesis of I-855.

To a mixture of intermediate 1-7 (25 mg, 38.99 μmol, 1 eq, HCl salt) and intermediate 1-8 (12.04 mg, 46.79 μmol, 1.2 eq) in DMF (1 mL) was added HOAt (5.31 mg, 38.99 μmol, 5.45 μL, 1 eq), EDCI (22.42 mg, 116.97 μmol, 3 eq), and NMM (19.72 mg, 194.95 μmol, 21.43 μL, 5 eq), then the mixture was stirred at 20° C. for 1 hour. The reaction mixture was diluted with water (10 mL) and extracted with EA (3 mL*3). The combined organic layers were concentrated under reduced pressure to give a residue. The crude product was purified by reversed-phase HPLC (0.1% FA condition). The eluent was concentrated under reduced pressure to remove ACN and then lyophilized to give I-855 (7.47 mg, 8.32 μmol, 21.34% yield, 99.130% purity, FA salt) as a white solid. LCMS (Method D): Retention time=0.402 min, (M+H)=844.5. SFC: Retention time=1.796 min. ¹H NMR (400 MHz, METHANOL-d₄) δ=8.54-8.49 (m, 1H), 8.37 (d, J=7.2 Hz, 1H), 7.98-7.93 (m, 1H), 7.91-7.81 (m, 2H), 7.54-7.45 (m, 1H), 7.38 (d, J=2.8 Hz, 1H), 7.19-7.15 (m, 1H), 4.58 (s, 1H), 4.52-4.44 (m, 1H), 4.39 (s, 3H), 4.11-3.84 (m, 2H), 3.81-3.66 (m, 4H), 3.58-3.50 (m, 3H), 3.26 (s, 3H), 2.87-2.77 (m, 2H), 2.43-2.26 (m, 2H), 2.07-2.01 (m, 1H), 1.96-1.83 (m, 3H), 1.79-1.53 (m, 9H), 1.43 (s, 10H), 1.32 (d, J=6.0 Hz, 2H), 1.28-1.17 (m, 5H), 1.12-0.97 (m, 2H). ¹⁹F NMR (377 MHz, METHANOL-d₄), δ=−120.762.

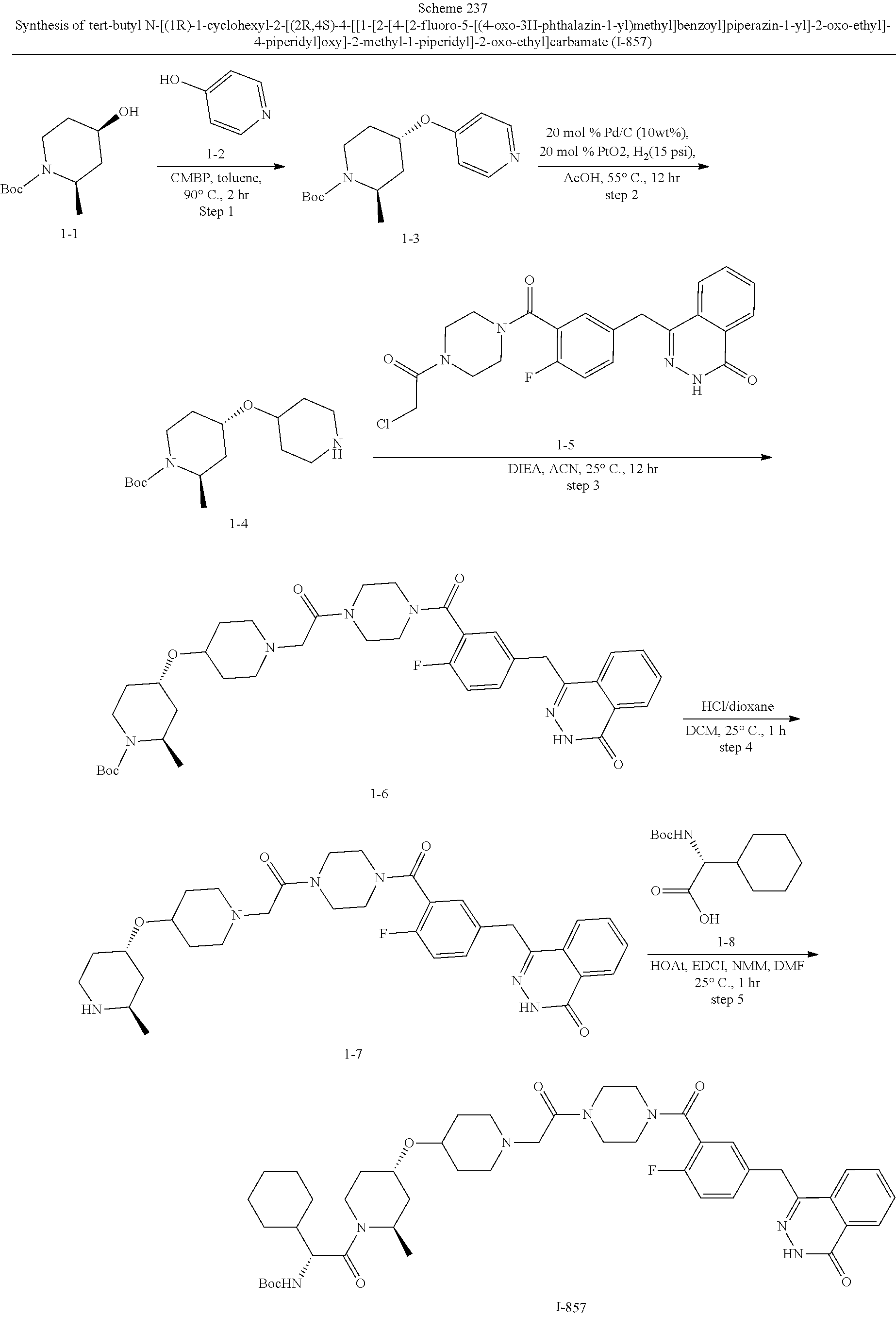

### Step 1: Synthesis of Intermediate 1-3

To a solution of intermediate 1-1 (1.7 g, 7.90 mmol, 1 eq) and intermediate 1-2 (750.94 mg, 7.90 mmol, 1 eq) in toluene (15 mL) was added CMBP (5.72 g, 23.69 mmol, 3 eq). The mixture was stirred at 90° C. for 12 hrs. The mixture was filtered and the filtrate was purified by reversed phase HPLC (0.1% FA condition). The eluent was concentrated under reduced pressure to remove ACN and then lyophilized to give product. Intermediate 1-3 (550 mg, 1.83 mmol, 23.23% yield, 97.5% purity) was obtained as a yellow solid. LCMS (Method D): Rt: 0.291 min, [M+H]⁺=293.2. ¹H NMR (400 MHz, METHANOL-d4) δ=8.41-8.36 (m, 2H), 7.16-7.02 (m, 2H), 4.96-4.88 (m, 1H), 4.62-4.49 (m, 1H), 4.15-4.02 (m, 1H), 3.19-3.03 (m, 1H), 2.22-2.12 (m, 1H), 2.10-2.01 (m, 1H), 1.79-1.76 (m, 1H), 1.57-1.55 (m, 1H), 1.50 (s, 1H), 1.48-1.46 (m, 9H), 1.29 (d, J=7.2 Hz, 3H). SFC: Rt: 3.735 min.

### Step 2: Synthesis of Intermediate 1-4

To a solution of intermediate 1-3 (100 mg, 342.03 μmol, 1 eq) in AcOH (2 mL) was added PtO₂ (50 mg) and Pd/C (50 mg, 10% purity). The flask was degassed and purged with H₂ 3 times, then the mixture was stirred at 55° C. for 12 hrs under H₂ (15 psi) atmosphere. The mixture was filtered and the filter cake was washed with MeOH (1 mL*3), then the filtrate was concentrated under reduced pressure to give a residue. To the mixture was added NH₃·H₂O (2 mL) to adjust pH to 8. The mixture was diluted with H₂O (2 mL) and extracted with DCM (1 mL*2). The combined organic layers were dried over Na₂SO₄, filtered and concentrated under reduced pressure to give intermediate 1-4 (60 mg, 201.06 μmol, 58.78% yield) as a yellow oil. LCMS (Method D): Rt: 0.287 min, [M+H]⁺=299.2.

### Step 3: Synthesis of Intermediate 1-6

To a solution of intermediate 1-4 (60 mg, 201.06 μmol, 1 eq) and intermediate 1-5 (71.23 mg, 160.85 μmol, 0.8 eq) in ACN (1 mL) was added DIEA (77.96 mg, 603.18 μmol, 105.06 μL, 3 eq). The mixture was stirred at 25° C. for 12 hrs. The mixture was diluted with H₂O (1 mL) and extracted with DCM (1 mL*2). The combined organic layers were dried over Na₂SO₄, filtered and concentrated under reduced pressure to give a residue. The residue was purified by flash silica gel chromatography (ISCO; 12 g SepaFlash Silica Flash Column, Eluent of 0˜15% MeOH/Ethyl acetate gradient @60 mL/min). The eluent was concentrated under reduced pressure to give product. Intermediate 1-6 (55 mg, 76.00 μmol, 37.80% yield, 97.4% purity) was obtained as a white solid. LCMS (Method D): Rt: 0.361 min, [M+H]⁺=705.3.

### Step 4: Synthesis of Intermediate 1-7

A solution of intermediate 1-6 (50 mg, 70.94 μmol, 1 eq) in DCM (0.5 mL) and HCl/dioxane (2 M, 0.5 mL) was stirred at 25° C. for 1 hr. The mixture was concentrated under reduced pressure to give intermediate 1-7 (50 mg, crude, HCl salt) as a yellow solid. LCMS (Method D): Rt: 0.251 min, [M+H]⁺=605.3.

### Step 5: Synthesis of I-857

To a solution of intermediate 1-8 (20.07 mg, 77.98 μmol, 1 eq) in DMF (0.8 mL) was added EDCI (44.85 mg, 233.95 μmol, 3 eq), HOAt (10.61 mg, 77.98 μmol, 10.91 μL, 1 eq), intermediate 1-7 (50 mg, 77.98 μmol, 1 eq, HCl salt) and NMM (39.44 mg, 389.91 μmol, 42.87 μL, 5 eq). The mixture was stirred at 25° C. for 1 hr. The mixture was filtered. The filtrate was purified by reversed phase HPLC (0.1% FA condition). The eluent was concentrated under reduced pressure to remove ACN and then lyophilized to give product. I-857 (20.51 mg, 23.04 μmol, 29.55% yield, 100% purity, FA salt) was obtained as a white solid. LCMS (Method D): Rt: 0.401 min, [M+H]⁺=844.3. ¹H NMR (400 MHz, METHANOL-d4) δ=8.48 (s, 1H), 8.37 (d, J=7.6 Hz, 1H), 7.99-7.92 (m, 1H), 7.91-7.80 (m, 2H), 7.54-7.45 (m, 1H), 7.41-7.34 (m, 1H), 7.21-7.12 (m, 1H), 4.68-4.46 (m, 1H), 4.39 (s, 2H), 4.36-4.26 (m, 1H), 4.04-3.70 (m, 4H), 3.69-3.56 (m, 3H), 3.56-3.46 (m, 3H), 3.43-3.37 (m, 1H), 3.28-3.22 (m, 1H), 2.96-2.82 (m, 2H), 2.64-2.35 (m, 2H), 2.14-1.99 (m, 1H), 1.98-1.86 (m, 3H), 1.84-1.50 (m, 9H), 1.43 (s, 10H), 1.36-0.91 (m, 10H). ¹⁹F NMR (376 MHz, METHANOL-d4) δ=−120.787 (s). SFC: Rt: 2.023 min.

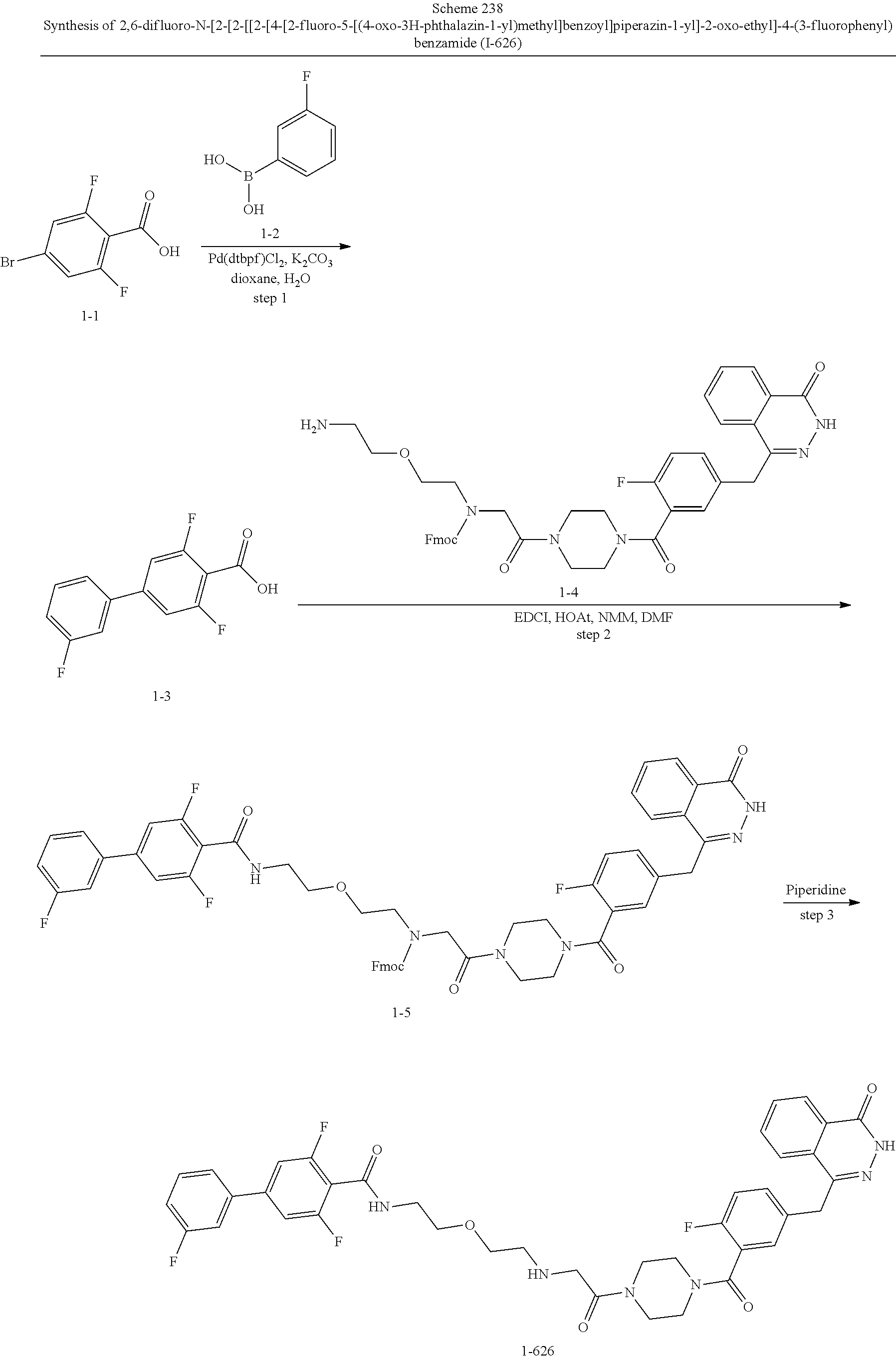

### Step 1: Synthesis of Intermediate 1-3

A mixture of Intermediate 1-1 (500 mg, 2.11 mmol, 1 eq), Intermediate 1-2 (324.71 mg, 2.32 mmol, 1.1 eq), cyclopentyl(diphenyl)phosphane;dichloropalladium;iron (154.37 mg, 210.97 μmol, 0.1 eq) and K₂CO₃ (583.15 mg, 4.22 mmol, 2 eq) in dioxane (4 mL) and H₂O (1 mL) was degassed and purged with N₂ 3 times, and then the mixture was stirred at 100° C. for 2 hr under N₂ atmosphere. The mixture was diluted with water (5 ml) and extracted with EtOAc (3 ml*3). The water layer was adjust to pH=3 with aq. HCl and extracted with EA. The organic layer was concentrated under vacuum. This residue was used in the next step without purification. Intermediate 1-3 (330 mg, crude) as a gray solid was obtained. ¹H NMR (400 MHz, METHANOL-d₄) δ=7.53-7.47 (m, 2H), 7.46-7.44 (m, 1H), 7.40-7.33 (m, 2H), 7.21-7.14 (m, 1H). ¹⁹F NMR (377 MHz, METHANOL-d₄) δ=−112.084, −114.301.

### Step 2: Synthesis of Intermediate 1-5

To a solution of Intermediate 1-3 (100 mg, 396.53 μmol, 1 eq), EDCI (152.03 mg, 793.06 μmol, 2 eq), HOAt (53.97 mg, 396.53 μmol, 55.47 μL, 1 eq) and NMM (200.54 mg, 1.98 mmol, 217.98 μL, 5 eq) in DMF (1.5 mL) was added Intermediate 1-4 (290.58 mg, 396.53 μmol, 1 eq). The mixture was stirred at 25° C. for 2 hr. The mixture was used in the next step without workup. Intermediate 1-5 (350 mg, crude) in DMF (1.5 mL) was obtained as a brown liquid. LCMS (Method G): Rt=0.721 min, [M+H]⁺=967.5.

### Step 3: Synthesis of I-626

To a solution of Intermediate 1-5 (350 mg, 361.96 μmol, 1 eq) in DMF (1.5 mL) was added piperidine (323.33 mg, 3.80 mmol, 0.375 mL, 10.49 eq), then the mixture was stirred at 25° C. for 0.5 h. The mixture was diluted with water (2 mL), extracted with EtOAc (2 mL×3). The organic layer was concentrated under vacuum. The residue was purified by Prep-HPLC (column: Waters Xbridge 150*25 mm*5 um; mobile phase: [water (ammonia hydroxide v/v)-ACN]; gradient: 20%-50% B over 10 min) and dried by lyophilization. I-626 (64.99 mg, 86.20 μmol, 23.82% yield, 98.78% purity) as an off-white solid was obtained. LCMS (Method G): Rt=0.572 min, [M+H]⁺=745.4. ¹H NMR (400 MHz, METHANOL-d₄) δ=8.37-8.33 (m, 1H), 7.96-7.90 (m, 1H), 7.88-7.79 (m, 2H), 7.50-7.45 (m, 3H), 7.42 (br d, J=12.0 Hz, 1H), 7.36 (br d, J=8.0 Hz, 3H), 7.18-7.10 (m, 2H), 4.36 (s, 2H), 3.75 (br s, 1H), 3.68 (br d, J=4.0 Hz, 1H), 3.62 (br d, J=8.0 Hz, 8H), 3.54 (br s, 2H), 3.47 (br s, 2H), 3.38 (br s, 1H), 3.24 (br s, 1H), 2.80 (br d, J=4.0 Hz, 2H). ¹⁹F NMR (377 MHz, METHANOL-d₄) δ=−114.211, −114.229, −114.586, −120.695.

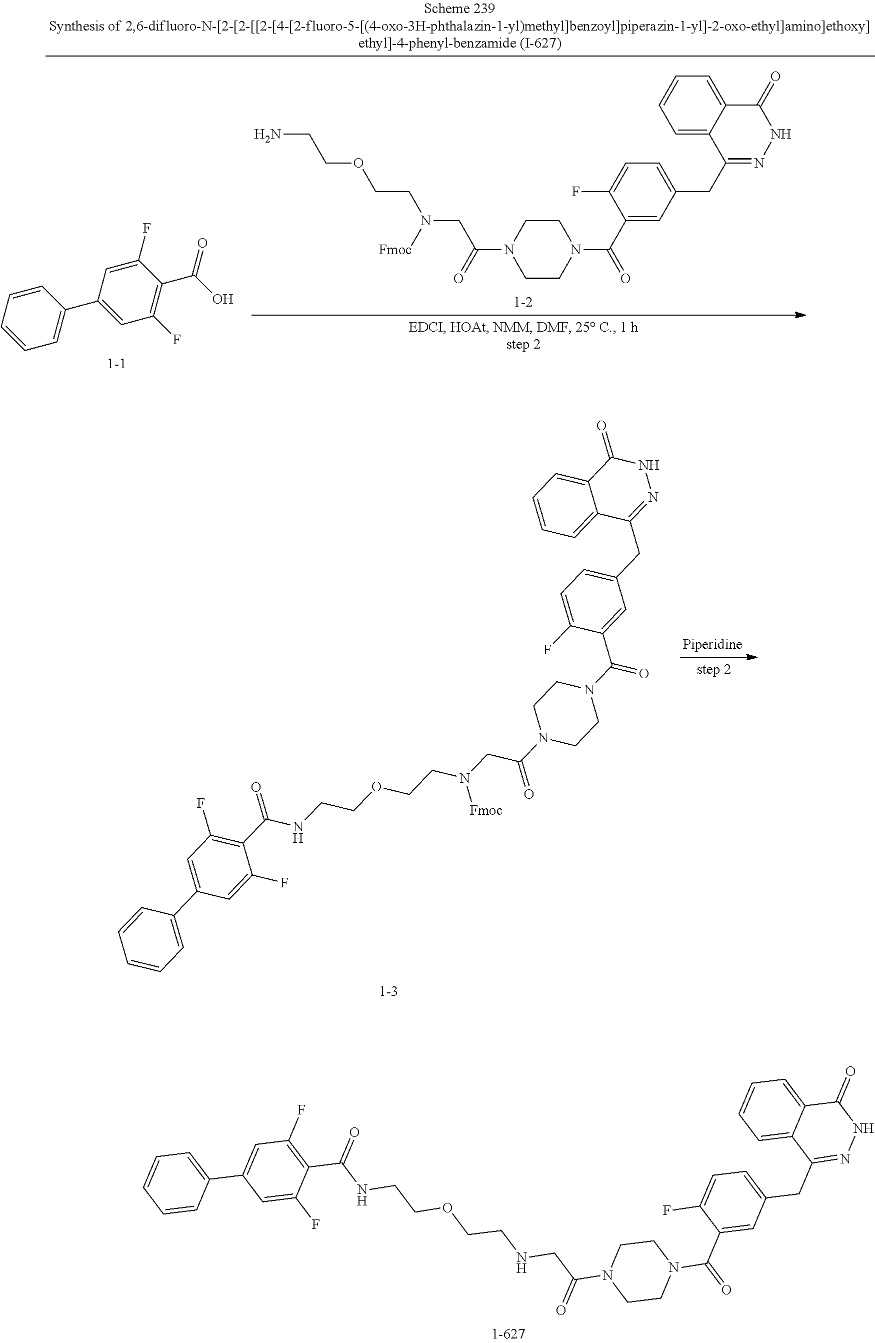

### Step 1: Synthesis of Intermediate 1-3

To a solution of Intermediate 1-1 (50 mg, 213.49 μmol, 1 eq) in DMF (1 mL) was added Intermediate 1-2 (156.45 mg, 213.49 μmol, 1 eq), EDCI (81.85 mg, 426.99 μmol, 2 eq), NMM (107.97 mg, 1.07 mmol, 117.36 μL, 5 eq) and HOAt (29.06 mg, 213.49 μmol, 29.87 μL, 1 eq). The mixture was stirred at 25° C. for 1 hr. The residue was used in the next step without work up. A solution of Intermediate 1-3 (200 mg, crude) in DMF (1 mL) as brown liquid was obtained. LCMS (Method G): Rt=0.697 min, [M+H]⁺=949.5.

### Step 2: Synthesis of I-627

To a solution of Intermediate 1-3 (200 mg, 210.75 μmol, 1 eq) in DMF (1 mL) was added piperidine (215.55 mg, 2.53 mmol, 0.25 mL, 12.01 eq). The mixture was stirred at 25° C. for 0.5 hr. The mixture was diluted with water (5 ml) and extracted with EtOAc (10 ml*3). The organic layer was concentrated under vacuum. The residue was purified by Prep-HPLC (column: Waters Xbridge 150*25 mm*5 um; mobile phase: [water (ammonia hydroxide v/v)-ACN]; gradient: 20%-50% B over 10 min) and dried by lyophilization. I-627 (27 mg, 35.63 μmol, 16.91% yield, 95.9% purity) was obtained as an off-white solid. LCMS (Method G): Rt=0.563 min, [M+H]⁺=727.4. SFC: Rt=1.788 min, ee %=100%. ¹H NMR (400 MHz, METHANOL-d₄) δ=8.38-8.33 (m, 1H), 7.95-7.91 (m, 1H), 7.87-7.82 (m, 2H), 7.66-7.61 (m, 2H), 7.49-7.39 (m, 4H), 7.32 (d, J=9.2 Hz, 3H), 7.16-7.09 (m, 1H), 4.36 (s, 2H), 3.83-3.74 (m, 1H), 3.71-3.59 (m, 8H), 3.59-3.40 (m, 5H), 3.40-3.35 (m, 1H), 3.26-3.19 (m, 1H), 2.80 (br d, J=4.4 Hz, 2H). ¹⁹F NMR (377 MHz, METHANOL-d₄) δ=−114.939, −120.696.

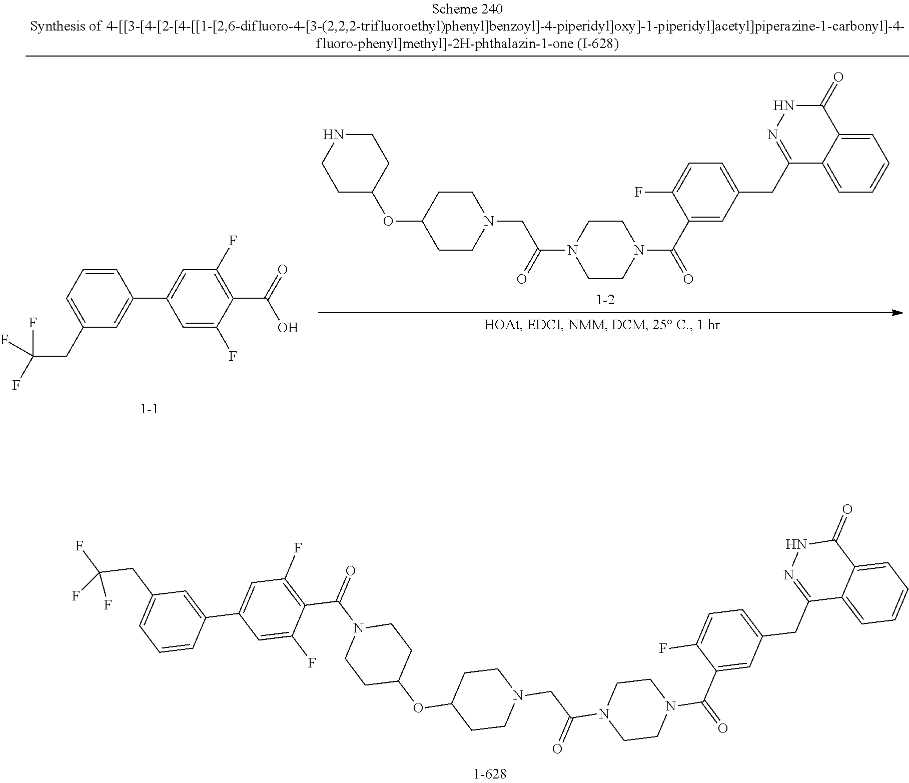 [SMILES: O=C(O)c1c(F)cc(-c2cccc(CC(F)(F)F)c2)cc1F ; O=C(CN1CCC(OC2CCNCC2)CC1)N1CCN(C(=O)c2cc(Cc3n[nH]c(=O)c4ccccc34)ccc2F)CC1 ; O=C(CN1CCC(OC2CCN(C(=O)c3c(F)cc(-c4cccc(CC(F)(F)F)c4)cc3F)CC2)CC1)N1CCN(C(=O)c2cc(Cc3n[nH]c(=O)c4ccccc34)ccc2F)CC1]

To a solution of intermediate 1-1 (50 mg, 158.12 μmol, 1 eq) in DCM (2 mL) was added EDCI (90.93 mg, 474.35 μmol, 3 eq), NMM (79.97 mg, 790.58 μmol, 86.92 μL, 5 eq) and HOAt (8.61 mg, 63.25 μmol, 8.85 μL, 1 eq) at 25° C. for 0.5 hr. And then intermediate 1-2 (93.40 mg, 158.12 μmol, 1 eq) was added into the mixture and stirred at 25° C. for 0.5 hr. The reaction mixture was diluted with water (30 mL) and extracted with EA (10 mL×3). The combined organic layers were washed with brine (10 mL×3) and dried over sodium sulfate, filtered and concentrated under reduced pressure to give a residue. The crude product was purified by reversed-phase HPLC (column: CD07-Daisogel SP-100-8-ODS-PK 150*25*10 um; mobile phase: [water (NH₄HCO₃)-ACN]; gradient: 40%-70% B over 10 min) and the eluent was concentrated under reduced pressure to remove ACN and then lyophilized to give I-628 (50.95 mg, 56.70 μmol, 35.86% yield, 98.922% purity) as a white solid. LCMS (Method D): Rt: 0.414 min, (M+H)=889.2. ¹H NMR (400 MHz, METHANOL-d4) δ=8.40-8.35 (m, 1H), 7.99-7.92 (m, 1H), 7.91-7.81 (m, 2H), 7.71-7.64 (m, 2H), 7.54-7.47 (m, 2H), 7.46-7.36 (m, 4H), 7.17 (s, 1H), 4.39 (s, 2H), 4.12-4.01 (m, 1H), 3.86-3.72 (m, 4H), 3.71-3.47 (m, 9H), 3.30-3.17 (m, 4H), 2.77 (s, 2H), 2.36-2.20 (m, 2H), 1.90 (d, J=13.2 Hz, 4H), 1.71-1.52 (m, 4H). ¹⁹F NMR (376 MHz, METHANOL-d4) δ=−67.429 (m), −115.056-−115.117 (m), −120.799 (s).

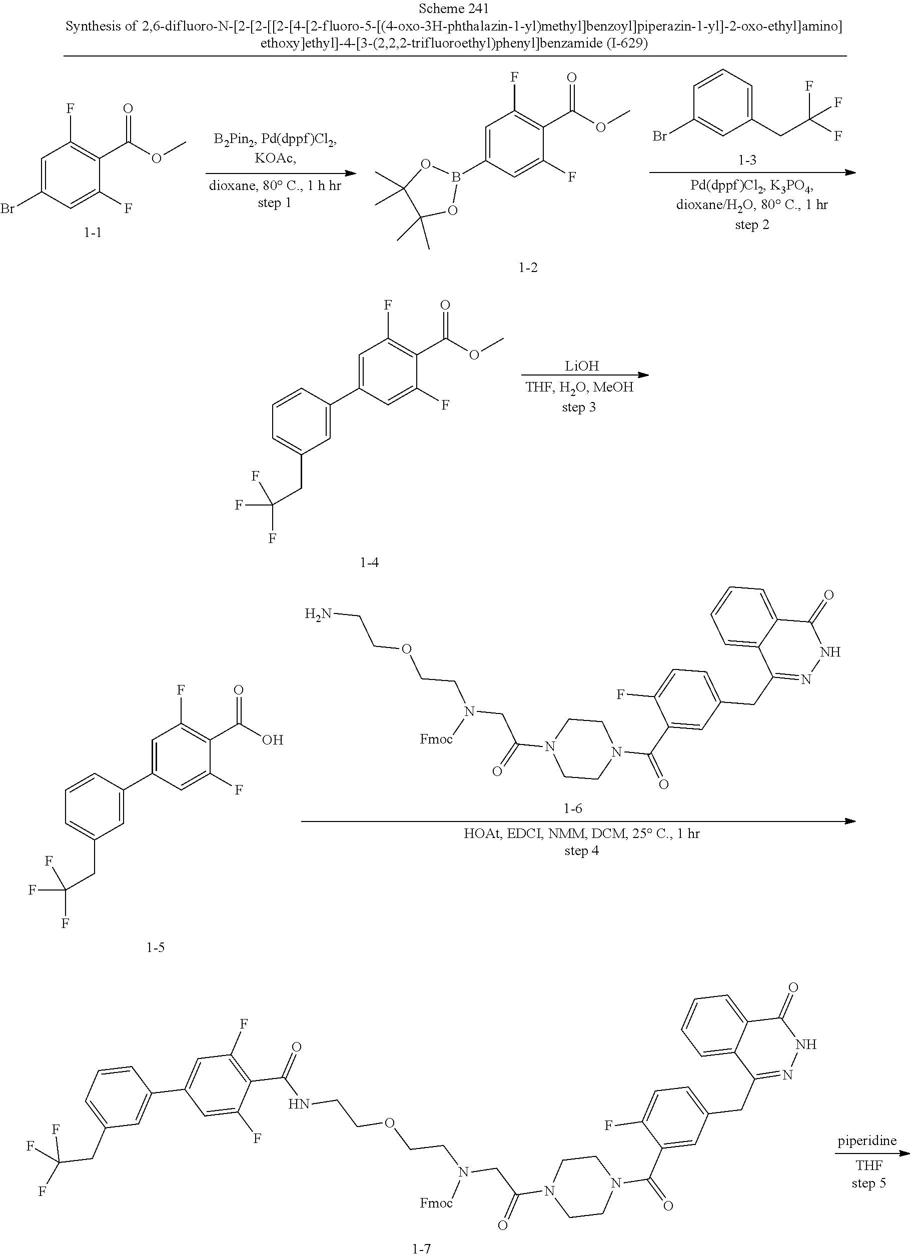

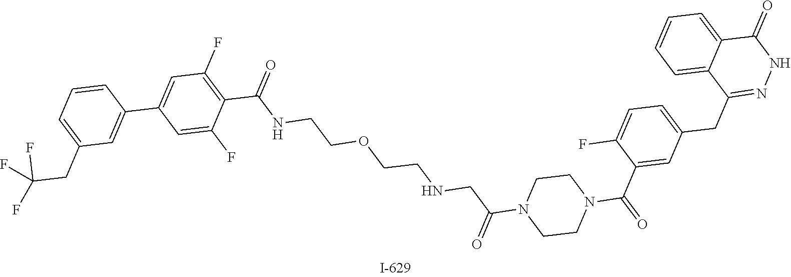 [SMILES: O=C(NCCOCCNCC(=O)N1CCN(C(=O)c2cc(Cc3n[nH]c(=O)c4ccccc34)ccc2F)CC1)c1c(F)cc(-c2cccc(CC(F)(F)F)c2)cc1F]

### Step 1: Synthesis of Intermediate 1-2

To a solution of intermediate 1-1 (303.48 mg, 1.20 mmol, 1.5 eq) in dioxane (5 mL) and Pin₂B2 (303.48 mg, 1.20 mmol, 1.5 eq) was added Pd(dppf)Cl₂ (58.30 mg, 79.67 μmol, 0.1 eq) and KOAc (234.58 mg, 2.39 mmol, 3 eq). The suspension was degassed and purged with N₂ 3 times. The mixture was stirred at 90° C. for 1 hr under N₂ atmosphere. The reaction mixture was diluted with water (30 mL) and extracted with EA (10 mL×3). The combined organic layers were washed with brine (10 mL×3) and dried over sodium sulfate, filtered and concentrated under reduced pressure to give intermediate 1-2 (200 mg, 670.94 μmol, 84.21% yield) as a brown oil. LCMS (Method D): Rt: 0.489 min, [M+H]⁺=299.0.

### Step 2: Synthesis of Intermediate 1-4

To a solution of intermediate 1-2 (180 mg, 603.85 μmol, 1 eq) and intermediate 1-3 (144.34 mg, 603.85 μmol, 1 eq) in H₂O (1 mL) and dioxane (3 mL) was added Pd(dppf)Cl₂ (44.18 mg, 60.38 μmol, 0.1 eq) and K₃PO₄ (250.36 mg, 1.81 mmol, 3 eq). The suspension was degassed and purged with N₂ 3 times. The mixture was stirred at 90° C. for 1 hr under N₂ atmosphere. The mixture was concentrated under reduced pressure to give a residue. The residue was purified by flash silica gel chromatography (ISCO; 40 g SepaFlash Silica Flash Column, Eluent of 0-20% DCM:MeOH gradient @40 mL/min). The eluent was concentrated under reduced pressure to give intermediate 1-4 (180 mg, 541.31 μmol, 89.64% yield, 99.315% purity) as a yellow oil. LCMS (Method D): Rt: 0.500 min, [M+H]⁺=330.9.

### Step 3: Synthesis of Intermediate 1-5

To a solution of intermediate 1-4 (180 mg, 545.04 μmol, 1 eq) in THF (0.8 mL), H₂O (0.2 mL) and MeOH (0.8 mL) was added LiOH·H₂O (68.62 mg, 1.64 mmol, 3 eq). The mixture was stirred at 40° C. for 0.5 hr. The mixture was concentrated to give a residue. The reaction mixture was diluted with water (30 mL). Then the mixture was adjusted to pH=6-7 with saturated CA aqueous solution. Then the reaction mixture was extracted with DCM (10 mL×3). The combined organic layers were washed with brine (10 mL×3) and dried over sodium sulfate, filtered and concentrated under reduced pressure to give intermediate 1-5 (160 mg, 505.97 μmol, 92.83% yield) as a white solid. LCMS (Method D): Rt: 0.435 min, [M+H]⁺=316.9.

### Step 4: Synthesis of Intermediate 1-7

To a solution of intermediate 1-5 (50 mg, 158.12 μmol, 1 eq) in DCM (2 mL) was added EDCI (90.93 mg, 474.35 μmol, 3 eq), NMM (79.97 mg, 790.58 μmol, 86.92 μL, 5 eq) and HOAt (21.52 mg, 158.12 μmol, 22.12 μL, 1 eq) at 25° C. for 0.5 hr. And then intermediate 1-6 (115.87 mg, 158.12 μmol, 1 eq) was added into the mixture and it was stirred at 25° C. for 0.5 hr. The reaction mixture was diluted with water (3 mL) and extracted with EA (5 mL×3). The combined organic layers were washed with brine (10 mL×3) and dried over sodium sulfate, filtered and concentrated under reduced pressure to give intermediate 1-7 (130 mg, 126.09 μmol, 79.75% yield) as a yellow oil. LCMS (Method D): Rt: 0.518 min, [M+H]⁺=1031.2.

### Step 5: Synthesis of I-629

To a solution of intermediate 1-7 (100 mg, 96.99 μmol, 1 eq) in THF (1 mL) was added piperidine (0.2 mL). The mixture was stirred at 25° C. for 0.5 hr. The reaction mixture was diluted with water (5 mL) and extracted with EA (5 mL×3). The combined organic layers were washed with brine (5 mL) and dried over sodium sulfate, filtered and concentrated under reduced pressure to give a residue. The crude product was purified by reversed-phase HPLC (column: CD07-Daisogel SP-100-8-ODS-PK 150*25*10 um; mobile phase: [water (NH4HCO3)-ACN]; gradient: 22%-52% B over 10 min) and the eluent was concentrated under reduced pressure to remove ACN and then lyophilized to give I-629 (50.04 mg, 61.35 μmol, 63.25% yield, 99.153% purity). LCMS (Method D): Rt: 0.379 min, [M+H]⁺=809.2. ¹H NMR (400 MHz, METHANOL-d4) δ=8.34 (d, J=7.6 Hz, 1H), 7.95-7.88 (m, 1H), 7.87-7.76 (m, 2H), 7.62 (s, 2H), 7.52-7.37 (m, 3H), 7.33 (d, J=8.8 Hz, 3H), 7.12 (s, 1H), 4.35 (s, 2H), 3.80-3.42 (m, 16H), 3.38 (s, 1H), 3.24 (s, 1H), 2.80 (d, J=4.8 Hz, 2H). ¹⁹F NMR (376 MHz, METHANOL-d4) δ=−67.302, −114.662, −120.599.

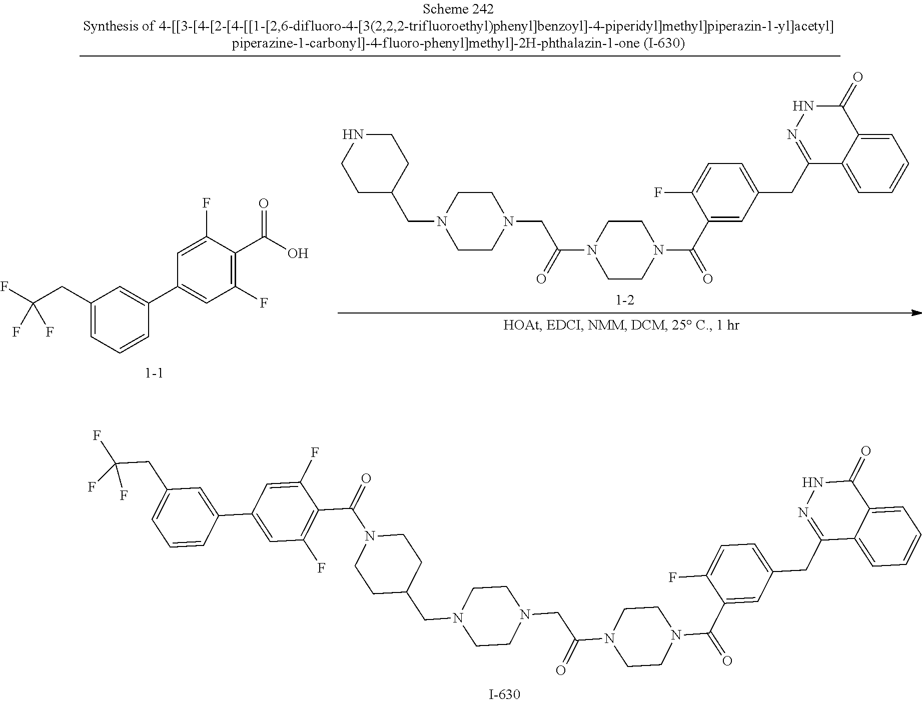 [SMILES: O=C(O)c1c(F)cc(-c2cccc(CC(F)(F)F)c2)cc1F ; O=C(CN1CCN(CC2CCNCC2)CC1)N1CCN(C(=O)c2cc(Cc3n[nH]c(=O)c4ccccc34)ccc2F)CC1 ; O=C(CN1CCN(CC2CCN(C(=O)c3c(F)cc(-c4cccc(CC(F)(F)F)c4)cc3F)CC2)CC1)N1CCN(C(=O)c2cc(Cc3n[nH]c(=O)c4ccccc34)ccc2F)CC1]

### Step 1: Synthesis of Intermediate 1-3

To a solution of intermediate 1-1 (50 mg, 158.12 μmol, 1 eq) in DCM (2 mL) was added EDCI (90.93 mg, 474.35 μmol, 3 eq), NMM (79.97 mg, 790.58 μmol, 86.92 μL, 5 eq) and HOAt (21.52 mg, 158.12 μmol, 22.12 μL, 1 eq) at 25° C. for 0.5 hr. Then intermediate 1-2 (93.24 mg, 158.12 μmol, 1 eq) was added into the mixture and stirred at 25° C. for 0.5 hr. The reaction mixture was diluted with water (5 mL) and extracted with EA (5 mL×3). The combined organic layers were washed with brine (5 mL) and dried over sodium sulfate, filtered and concentrated under reduced pressure to give a residue. The crude product was purified by reversed-phase HPLC (column: CD07-Daisogel SP-100-8-ODS-PK 150*25*10 um; mobile phase: [water (NH₄HCO3)-ACN]; gradient: 39%-69% B over 10 min) and the eluent was concentrated under reduced pressure to remove ACN and then lyophilized to give I-630 (24.12 mg, 27.10 μmol, 17.14% yield, 99.754% purity) as a white solid. LCMS (Method D): Rt: 0.388 min, [M+H]⁺=888.2. ¹H NMR (400 MHz, METHANOL-d4) δ=8.40-8.33 (m, 1H), 7.99-7.92 (m, 1H), 7.91-7.79 (m, 2H), 7.69-7.63 (m, 2H), 7.53-7.46 (m, 2H), 7.45-7.33 (m, 4H), 7.16 (s, 1H), 4.69 (d, J=12.8 Hz, 1H), 4.39 (s, 2H), 3.83-3.69 (m, 3H), 3.68-3.49 (m, 6H), 3.33 (s, 1H), 3.29-3.11 (m, 4H), 2.92-2.93 (m, 1H), 2.51 (s, 8H), 2.26-2.24 (m, 2H), 1.99-1.85 (m, 2H), 1.85-1.77 (m, 1H), 1.28-1.08 (m, 2H). ¹⁹F NMR (376 MHz, METHANOL-d4) δ=−67.429, −115.102, −120.802.

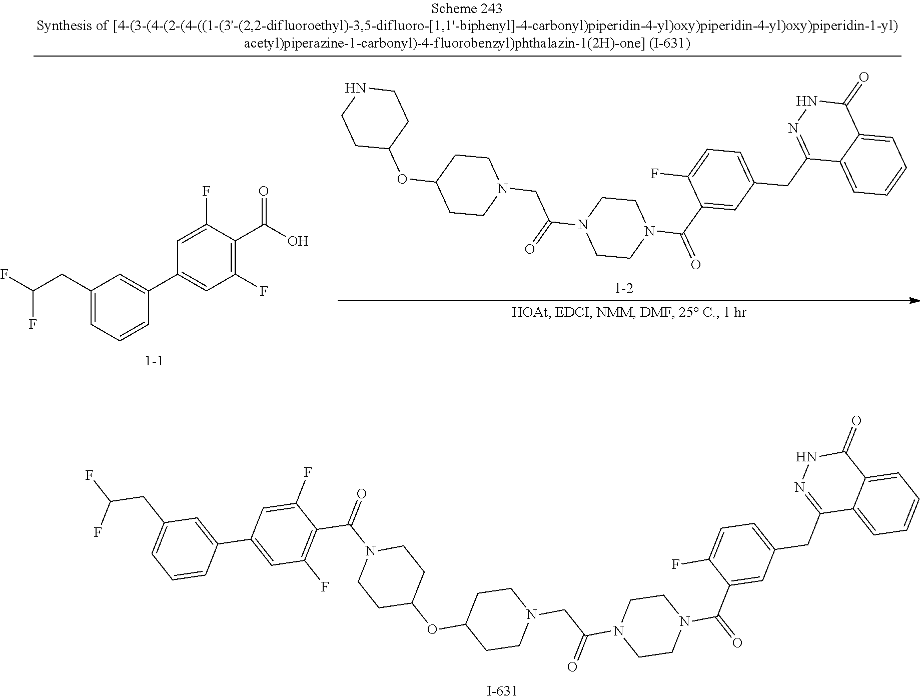 [SMILES: O=C(O)c1c(F)cc(-c2cccc(CC(F)F)c2)cc1F ; O=C(CN1CCC(OC2CCNCC2)CC1)N1CCN(C(=O)c2cc(Cc3n[nH]c(=O)c4ccccc34)ccc2F)CC1 ; O=C(CN1CCC(OC2CCN(C(=O)c3c(F)cc(-c4cccc(CC(F)F)c4)cc3F)CC2)CC1)N1CCN(C(=O)c2cc(Cc3n[nH]c(=O)c4ccccc34)ccc2F)CC1]

To a solution of intermediate 1-1 (84.12 mg, 134.12 μmol, 1 eq, HCl salt) in DMF (1 mL) was added HOAt (18.26 mg, 134.12 μmol, 18.76 μL, 1 eq), EDCI (77.14 mg, 402.37 μmol, 3 eq) and NMM (67.83 mg, 670.62 μmol, 73.73 μL, 5 eq). The mixture was stirred at 25° C. for 1 hr. The reaction mixture was diluted with H₂O (5 mL) and extracted with EA (5 mL*3). The combined organic layers were concentrated under reduced pressure to give a residue. The residue was purified by reversed phase HPLC (0.1% FA condition), the eluent was concentrated and lyophilized to give the desired product. I-631 (49.53 mg, 53.75 μmol, 40.08% yield, 99.512% purity, FA salt) was obtained as a white solid. LCMS (Method D): Retention time: 0.407 min, [M+H]⁺=871.3. ¹H NMR (400 MHz, METHANOL-d4) δ=8.45 (s, 1H), 8.37 (d, J=7.6 Hz, 1H), 7.98-7.92 (m, 1H), 7.90-7.80 (m, 2H), 7.60 (d, J=2.0 Hz, 2H), 7.52-7.44 (m, 2H), 7.42-7.34 (m, 4H), 7.21-7.13 (m, 1H), 6.25-5.85 (m, 1H), 4.39 (s, 2H), 4.11-4.01 (m, 1H), 3.81 (s, 2H), 3.75 (d, J=5.2 Hz, 1H), 3.73-3.56 (m, 7H), 3.55-3.44 (m, 2H), 3.35 (s, 2H), 3.29-3.18 (m, 3H), 3.14-2.96 (m, 2H), 2.83-2.60 (m, 2H), 1.97 (d, J=3.2 Hz, 3H), 1.87-1.53 (m, 5H). ¹⁹F NMR (376 MHz, CHLOROFORM-d) δ=−115.23, −117.01, 120.73.

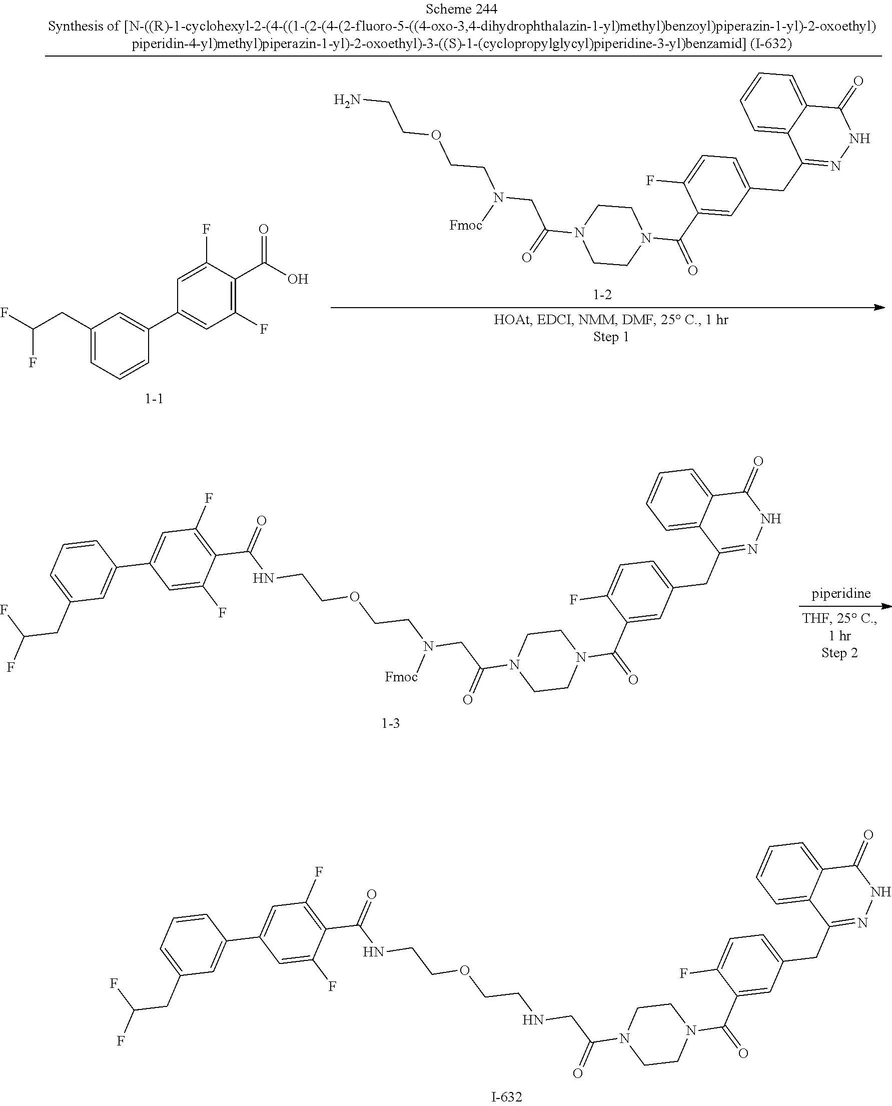

### Step 1: Synthesis of Intermediate 1-3.

To a solution of intermediate 1-1 (70 mg, 234.72 μmol, 1 eq) and intermediate 1-2 (180.56 mg, 234.72 μmol, 1 eq, HCl salt) in DMF (1 mL) was added HOAt (31.95 mg, 234.72 μmol, 32.83 μL, 1 eq), EDCI (134.99 mg, 704.15 μmol, 3 eq) and NMM (118.70 mg, 1.17 mmol, 129.03 μL, 5 eq). The mixture was stirred at 25° C. for 0.5 hr. The reaction mixture was diluted with H₂O (5 mL) and extracted with EA (5 mL*3). The combined organic layers were concentrated under reduced pressure to give a residue. The residue was purified by reversed-phase HPLC (Neutral condition), the eluent was concentrated and lyophilized to give the desired product. Intermediate 1-3 (150 mg, 145.53 μmol, 62.00% yield, 98.284% purity) was obtained as a yellow solid. LCMS (Method D): Retention time: 0.470 min, [M+H]⁺=1013.3. ¹H NMR (400 MHz, METHANOL-d4) δ=8.48 (s, 1H), 8.37-8.29 (m, 1H), 7.93-7.86 (m, 1H), 7.85-7.67 (m, 4H), 7.66-7.58 (m, 1H), 7.54-7.40 (m, 5H), 7.38-7.31 (m, 4H), 7.30-7.20 (m, 4H), 7.17-7.02 (m, 1H), 6.22-5.87 (m, 1H), 4.57-4.48 (m, 2H), 4.37-4.29 (m, 2H), 4.27-4.10 (m, 2H), 4.00-3.89 (m, 1H), 3.74-3.57 (m, 3H), 3.56-3.49 (m, 5H), 3.43 (d, J=4.0 Hz, 2H), 3.27-3.11 (m, 7H). ¹⁹F NMR (376 MHz, METHANOL-d4) δ=−114.62, −116.91, −120.68.

### Step 2: Synthesis of I-632

To a solution of intermediate 1-3 (70 mg, 66.10 μmol, 1 eq, FA salt) in THF (0.8 mL) was added piperidine (0.1 mL). The mixture was stirred at 25° C. for 0.5 hr. The reaction mixture was concentrated under reduced pressure to give a residue. The residue was purified by reversed-phase HPLC (neutral condition), the eluent was concentrated and lyophilized to give the desired product. I-632 (24.58 mg, 31.08 μmol, 47.03% yield, 100% purity) was obtained as a white solid. LCMS (Method D): Retention time: 0.375 min, [M+H]⁺=791.2. ¹H NMR (400 MHz, METHANOL-d4) δ=8.40-8.31 (m, 1H), 7.95-7.91 (m, 1H), 7.88-7.79 (m, 2H), 7.60-7.54 (m, 2H), 7.49-7.39 (m, 2H), 7.38-7.30 (m, 4H), 7.18-7.09 (m, 1H), 6.25-5.86 (m, 1H), 4.36 (s, 2H), 3.75 (s, 1H), 3.72-3.56 (m, 9H), 3.55 (s, 2H), 3.49-3.43 (m, 2H), 3.38 (s, 1H), 3.25-3.19 (m, 2H), 3.19-3.02 (m, 1H), 2.80 (d, J=4.8 Hz, 2H). ¹⁹F NMR (376 MHz, METHANOL-d4) δ=−114.85, −116.95, −120.67.

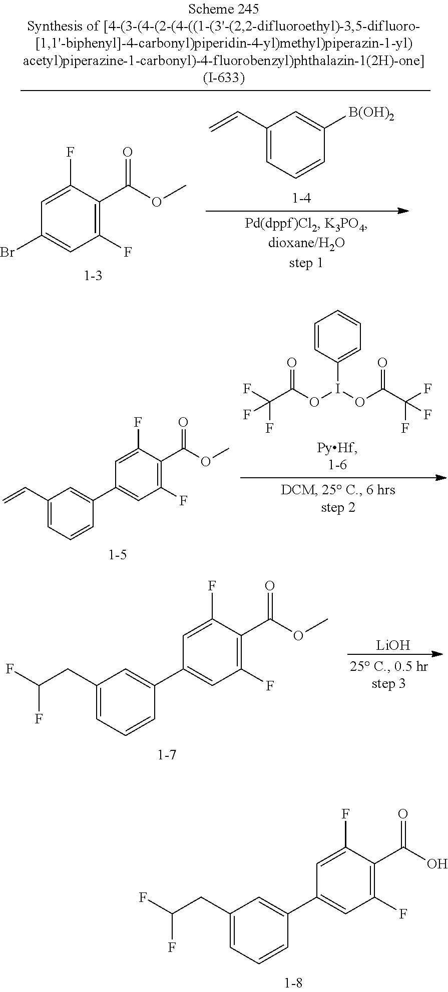

### Step 1: Synthesis of Intermediate 1-5.

A mixture of intermediate 1-3 (1 g, 3.98 mmol, 1 eq), intermediate 1-4 (648.39 mg, 4.38 mmol, 1.1 eq), K₃PO₄ (2.54 g, 11.95 mmol, 3 eq), Pd(dppf)C12 (291.49 mg, 398.37 μmol, 0.1 eq) in dioxane (10 mL) and H₂O (1 mL) was degassed and purged with N₂ 3 times, and then the mixture was stirred at 80° C. for 1 hr under N₂ atmosphere. The reaction mixture was diluted with H₂O (10 mL) and extracted with EA (10 mL*3). The combined organic layers were concentrated under reduced pressure to give a residue. The residue was purified by flash silica gel chromatography (ISCO; 20 g SepaFlash Silica Flash Column, Eluent of 0-15% Ethyl acetate/Petroleum ether gradient @60 mL/min), the eluent was concentrated under reduced pressure to give a residue. Intermediate 1-5 (900 mg, 2.78 mmol, 69.89% yield, 84.842% purity) was obtained as a yellow oil. LCMS (Method D): Retention time: 0.564 min, [M+H]⁺=274.9. ¹H NMR (400 MHz, CHLOROFORM-d) δ=7.57 (s, 1H), 7.52-7.43 (m, 3H), 7.23-7.17 (m, 2H), 6.84-6.72 (m, 1H), 5.89-5.76 (m, 1H), 5.37-5.31 (m, 1H), 3.98 (d, J=2.8 Hz, 3H). ¹⁹F NMR (376 MHz, CHLOROFORM-d) δ=−109.51.

### Step 2: Synthesis of Intermediate 1-7.

A solution of intermediate 1-6 (1.41 g, 3.28 mmol, 1 eq) and HF pyridine (6.50 g, 65.63 mmol, 5.91 mL, 20 eq) in DCM (5 mL) was stirred at 0° C. for 0.1 hr. Then intermediate 1-5 (900 mg, 3.28 mmol, 1 eq) in DCM (5 mL) was added into the mixture, then the mixture was stirred at 25° C. for 6 hrs. The reaction mixture was diluted with H₂O (10 mL) and extracted with DCM (15 mL*3). The combined organic layers were washed with brine (15 mL*3), dried over Na₂SO₄, filtered and concentrated under reduced pressure to give a residue. The residue was purified by flash silica gel chromatography (ISCO; 20 g SepaFlash Silica Flash Column, Eluent of 0˜3% Ethyl acetate/Petroleum ethergradient @60 mL/min), and the eluent was concentrated under reduced pressure to give a residue. Intermediate 1-7 (320 mg, 922.07 μmol, 28.10% yield, 89.976% purity) was obtained as a yellow oil. LCMS (Method D): Retention time: 0.479 min, [M+H]⁺=313.0. ¹H NMR (400 MHz, CHLOROFORM-d) δ=7.53-7.43 (m, 3H), 7.34 (d, J=7.2 Hz, 1H), 7.21-7.16 (m, 2H), 6.15-5.76 (m, 1H), 3.98 (s, 3H), 3.30-3.15 (m, 2H). ¹⁹F NMR (376 MHz, CHLOROFORM) δ=−109.39, −114.99.

### Step 3: Synthesis of Intermediate 1-8.

To a solution of intermediate 1-7 (300 mg, 960.74 μmol, 1 eq) in THF (1 mL), MeOH (1 mL), H₂O (1 mL) was added LiOH·H₂O (120.95 mg, 2.88 mmol, 3 eq). The mixture was stirred at 25° C. for 0.5 hr. The mixture was adjusted to pH=5 with 1M HCl and extracted with EA (15 mL*3), the organic layers were dried over Na₂SO₄, filtered and concentrated to give a residue. The crude product was used in the next step without further purification. Intermediate 1-8 (270 mg, 822.89 μmol, 85.65% yield, 90.893% purity) was obtained as a pale yellow solid. LCMS (Method D): Retention time: 0.423 min, [M+H]⁺=299.0. ¹H NMR (400 MHz, METHANOL-d4) δ=7.61 (s, 2H), 7.49-7.43 (m, 1H), 7.39-7.31 (m, 3H), 6.25-5.90 (m, 1H), 3.29-3.17 (m, 2H). ¹⁹F NMR (376 MHz, METHANOL-d4) δ=−112.29, −116.98.

### Step 4: Synthesis of Intermediate 1-2.

To a solution of intermediate 1-1 (500 mg, 724.83 μmol, 1 eq) in DCM (3 mL) was added HCl/dioxane (2 M, 3 mL). The mixture was stirred at 25° C. for 0.5 hr. The reaction mixture was concentrated under reduced pressure to give product. Intermediate 1-2 (450 mg, 718.66 μmol, 99.15% yield, HCl salt) was obtained as a pale yellow solid. LCMS (Method D): Retention time: 0.234 min, [M+H]⁺=596.2.

### Step 5: Synthesis of I-633

To a solution of intermediate 1-8 (50 mg, 167.65 μmol, 1 eq) and intermediate 1-2 (104.98 mg, 167.65 μmol, 1 eq, HCl salt) in DMF (1 mL) was added HOAt (22.82 mg, 167.65 μmol, 23.45 μL, 1 eq), EDCI (96.42 mg, 502.96 μmol, 3 eq) and NMM (84.79 mg, 838.27 μmol, 92.16 μL, 5 eq). The mixture was stirred at 25° C. for 1 hr. The reaction mixture was diluted with H₂O (5 mL) and extracted with EA (5 mL*3). The combined organic layers were dried over Na₂SO₄, filtered and concentrated under reduced pressure to give a residue. The residue was purified by reversed phase HPLC (0.1% FA condition), the eluent was concentrated and lyophilized to give the desired product. I-633 (21.08 mg, 22.95 μmol, 13.69% yield, 99.737% purity, FA salt) was obtained as a white solid. LCMS (Method D): Retention time: 0.377 min, [M+H]⁺=870.3. ¹H NMR (400 MHz, METHANOL-d4) δ=8.43 (s, 1H), 8.36 (d, J=8.0 Hz, 1H), 7.99-7.91 (m, 1H), 7.90-7.77 (m, 2H), 7.61 (d, J=1.6 Hz, 2H), 7.53-7.44 (m, 2H), 7.42-7.34 (m, 4H), 7.20-7.11 (m, 1H), 6.26-5.89 (m, 1H), 4.70 (d, J=12.8 Hz, 1H), 4.38 (s, 2H), 3.83-3.71 (m, 2H), 3.70-3.59 (m, 3H), 3.51 (d, J=4.8 Hz, 2H), 3.46-3.33 (m, 3H), 3.28 (d, J=4.4 Hz, 1H), 3.25-3.18 (m, 2H), 2.97-2.87 (m, 1H), 2.86-2.58 (m, 8H), 2.54-2.40 (m, 2H), 2.04-1.90 (m, 2H), 1.82 (d, J=12.8 Hz, 1H), 1.31-1.12 (m, 2H). ¹⁹F NMR (376 MHz, METHANOL-d4) δ=−115.13, −116.99, −120.77.

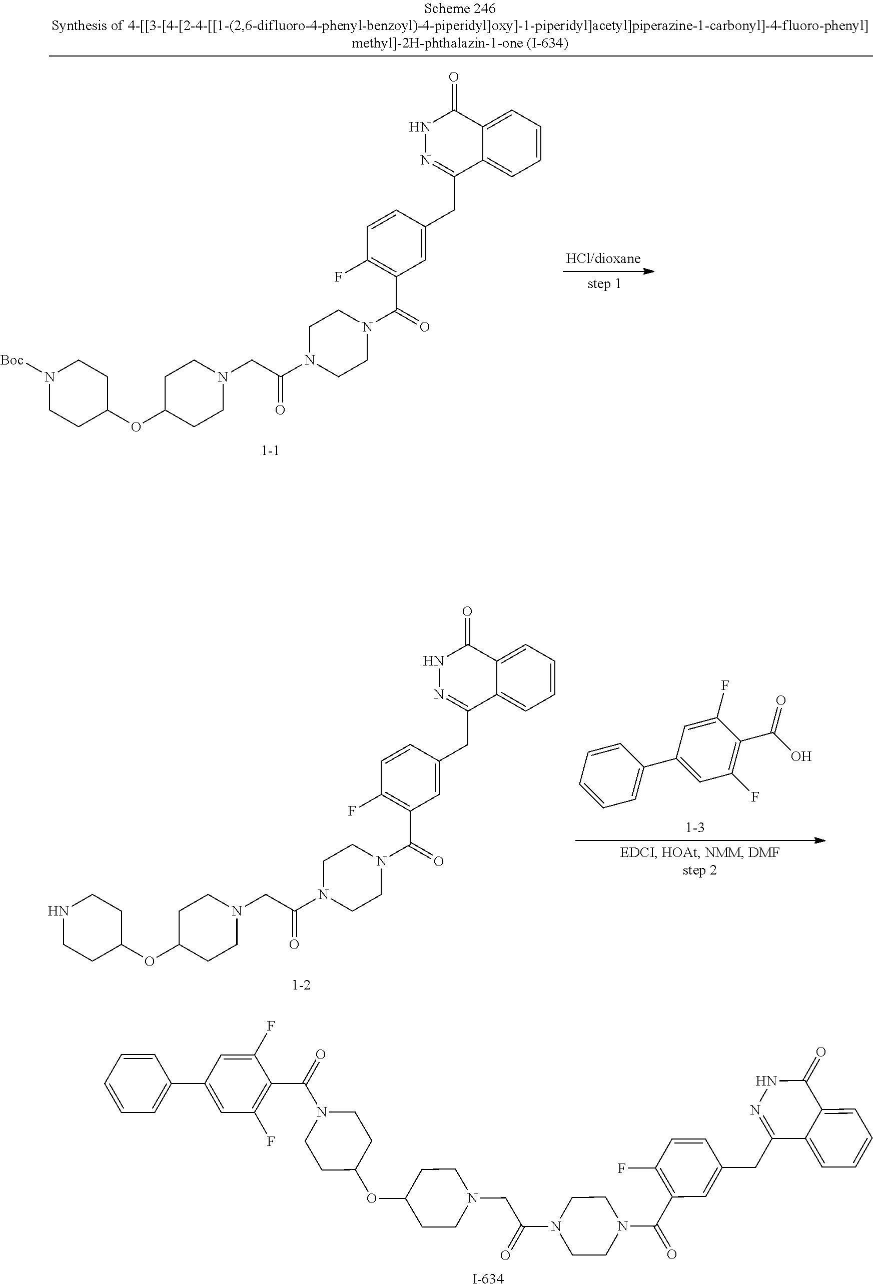 [SMILES: CC(C)(C)OC(=O)N1CCC(OC2CCN(CC(=O)N3CCN(C(=O)c4cc(Cc5n[nH]c(=O)c6ccccc56)ccc4F)CC3)CC2)CC1 ; O=C(CN1CCC(OC2CCNCC2)CC1)N1CCN(C(=O)c2cc(Cc3n[nH]c(=O)c4ccccc34)ccc2F)CC1 ; O=C(O)c1c(F)cc(-c2ccccc2)cc1F ; O=C(CN1CCC(OC2CCN(C(=O)c3c(F)cc(-c4ccccc4)cc3F)CC2)CC1)N1CCN(C(=O)c2cc(Cc3n[nH]c(=O)c4ccccc34)ccc2F)CC1]

### Step 1: Synthesis of Intermediate 1-2

To a solution of Intermediate 1-1 (100 mg, 144.76 μmol, 1 eq) in DCM (1 mL) was added HCl/dioxane (2 M, 2 mL, 27.63 eq). The mixture was stirred at 25° C. for 30 min. The reaction mixture was concentrated under vacuum. Intermediate 1-2 (80 mg, crude, HCl salt) was obtained as a light yellow oil and was used without further purification. LCMS (Method E): Rt=0.356 min, [M+H]⁺=591.3.

### Step 2: Synthesis of I-634

To a solution of Intermediate 1-2 (80 mg, 127.56 μmol, 1 eq, HCl salt), EDCI (48.91 mg, 255.12 μmol, 2 eq), HOAt (17.36 mg, 127.56 μmol, 17.84 μL, 1 eq) and NMM (64.51 mg, 637.81 μmol, 70.12 μL, 5 eq) in DMF (1 mL) was added Intermediate 1-3 (29.87 mg, 127.56 μmol, 1 eq). The mixture was stirred at 25° C. for 2 hr. The mixture was diluted with water (2 mL), extracted with EtOAc (2 mL×3). The organic layer was concentrated under vacuum. The residue was purified by Prep-HPLC (column: Waters Xbridge 150*25 mm*5 um; mobile phase: [water (NH₄HCO₃)-ACN]; gradient: 38%-68% B over 9 min) and dried by lyophilization. I-634 (31.56 mg, 39.11 μmol, 30.66% yield) as a white solid was obtained. LCMS (Method G): Rt=0.641 min, [M+H]⁺=807.4. ¹H NMR (400 MHz, METHANOL-d₄) δ=8.36-8.33 (m, 1H), 7.93 (br d, J=8.0 Hz, 1H), 7.88-7.83 (m, 1H), 7.83-7.77 (m, 1H), 7.66 (br d, J=8.0 Hz, 2H), 7.49-7.45 (m, 3H), 7.44-7.41 (m, 1H), 7.40-7.35 (m, 3H), 7.18-7.11 (m, 1H), 4.36 (s, 2H), 4.09-4.02 (m, 1H), 3.85-3.68 (m, 5H), 3.65 (br s, 1H), 3.60-3.48 (m, 5H), 3.30-3.26 (m, 2H), 3.24 (br s, 1H), 3.18 (s, 1H), 2.76 (br s, 2H), 2.25 (br d, J=8.0 Hz, 2H), 1.87 (br d, J=12.0 Hz, 4H), 1.65-1.51 (m, 4H. ¹⁹F NMR (377 MHz, METHANOL-d₄) δ=−115.099, −115.224, −120.624, −120.665.

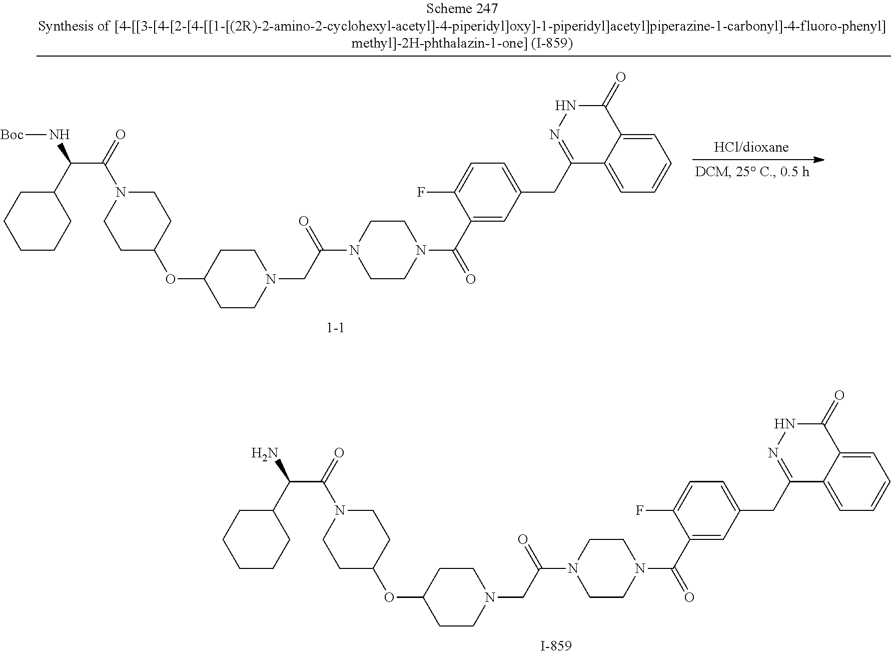 [SMILES: CC(C)(C)OC(=O)N[C@@H](C(=O)N1CCC(OC2CCN(CC(=O)N3CCN(C(=O)c4cc(Cc5n[nH]c(=O)c6ccccc56)ccc4F)CC3)CC2)CC1)C1CCCCC1 ; N[C@@H](C(=O)N1CCC(OC2CCN(CC(=O)N3CCN(C(=O)c4cc(Cc5n[nH]c(=O)c6ccccc56)ccc4F)CC3)CC2)CC1)C1CCCCC1]

To a solution of intermediate 1-1 (7.4 g, 8.92 mmol, 1 eq) in DCM (18 mL) was added HCl/dioxane (2 M, 37.00 mL, 8.30 eq). The mixture was stirred at 25° C. for 0.5 h. The reaction mixture was concentrated under reduced pressure to give a crude product. The residue (70 mg) was purified by prep-HPLC (column: CD01-Phenomenex luna C18 150*25*10 um; mobile phase: [water (FA)-ACN]; gradient: 1%-31% B over 10 min) and lyophilized to give the product. I-859 (32.81 mg, 41.51 μmol, 45.45% yield, 98.174% purity, FA salt) was obtained as a white solid. LCMS (Method D): Retention time: 0.264 min, [M+H]⁺=730.4. SFC: Retention time: 1.551 min. ¹H NMR (400 MHz, METHANOL-d4) δ=8.42 (s, 1H), 8.39-8.35 (m, 1H), 7.98-7.92 (m, 1H), 7.91-7.81 (m, 2H), 7.50 (d, J=2.4 Hz, 1H), 7.37 (t, J=7.2 Hz, 1H), 7.17 (t, J=9.2 Hz, 1H), 4.39 (s, 2H), 4.27 (d, J=5.2 Hz, 1H), 3.86-3.65 (m, 8H), 3.65-3.58 (m, 2H), 3.56-3.43 (m, 3H), 3.35 (s, 2H), 3.26 (d, J=3.6 Hz, 1H), 3.15-2.98 (m, 2H), 2.82-2.59 (m, 2H), 2.04-1.89 (m, 3H), 1.88-1.79 (m, 4H), 1.78-1.66 (m, 5H), 1.64-1.51 (m, 2H), 1.38-1.26 (m, 2H), 1.26-1.09 (m, 3H). ¹⁹F NMR: (377 MHz, METHANOL-d₄) δ=−120.792 (s).

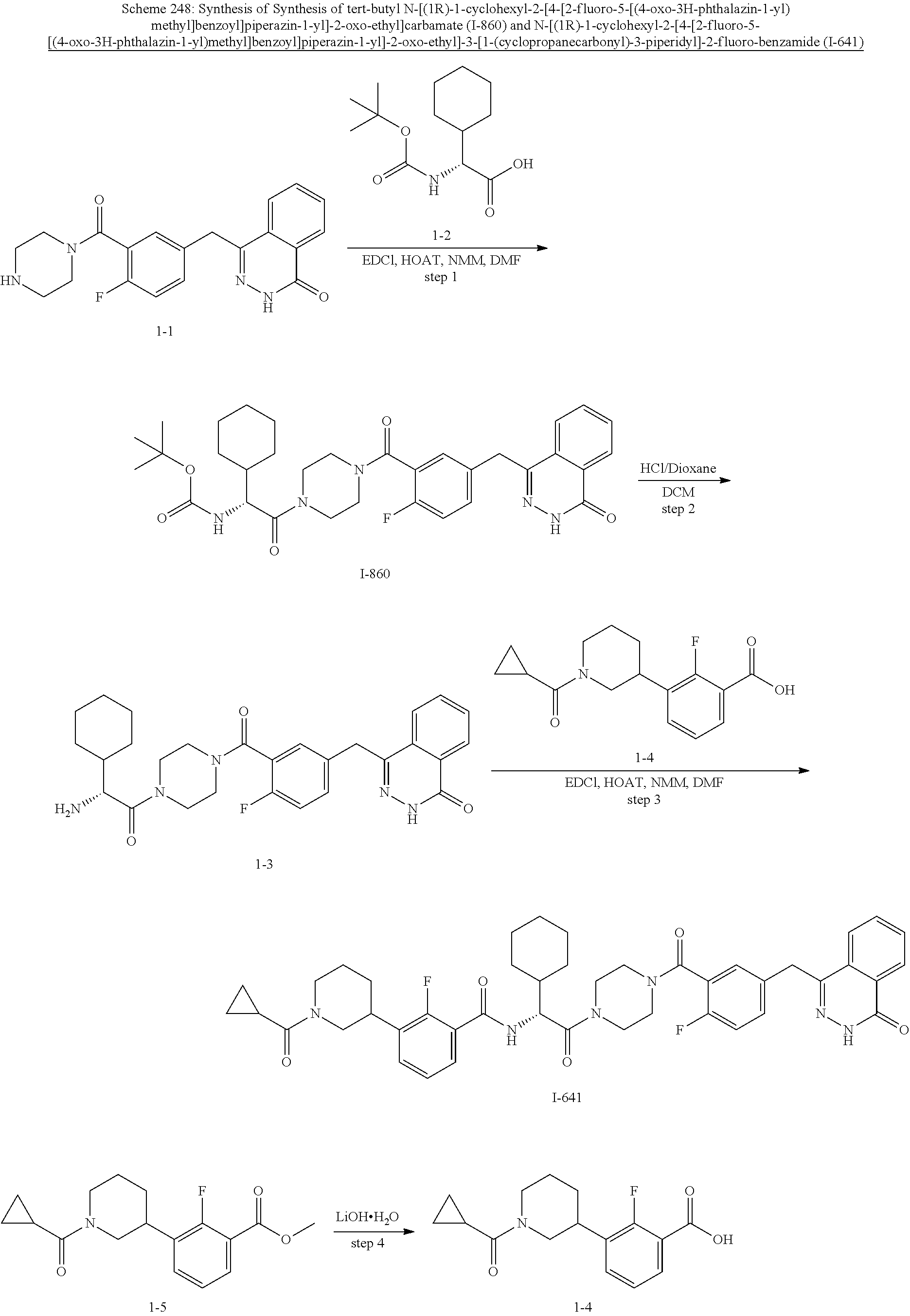

### Step 1: Synthesis of I-860

To a mixture of Intermediate 1-1 (0.1 g, 272.93 μmol, 1 eq), Intermediate 1-2 (70.23 mg, 272.93 mol, 1 eq) and HOAt (37.15 mg, 272.93 μmol, 38.18 μL, 1 eq) in DMF (1 mL) was added EDCI (104.64 mg, 545.87 μmol, 2 eq) and NMM (138.04 mg, 1.36 mmol, 150.04 μL, 5 eq). The mixture was stirred at 25° C. for 1 hr. The mixture was poured into water (5 mL) and filtered, and the filter cake was dried by lyophilization to afford the product (120 mg, 198.12 μmol, 72.59% yield) as a white solid. 30 mg of product was purified by reversed-phase HPLC (column: Waters Xbridge 150*25 mm*5 um; mobile phase: [water (NH₄HCO₃)-ACN]; gradient: 40%-70% B over 9 min) and concentrated under vacuum to remove MeCN and dried by lyophilization. I-860 (23.17 mg, 36.84 μmol, 74.38% yield, 96.31% purity) was obtained as a white solid. LCMS (Method G): Rt=0.644 min, [M+H]⁺=606.4. SFC: Rt=2.384 min. ¹H NMR (400 MHz, DMSO-d₆) δ=12.60 (s, 1H), 8.26 (d, J=7.6 Hz, 1H), 8.01-7.94 (m, 1H), 7.93-7.86 (m, 1H), 7.86-7.79 (m, 1H), 7.48-7.34 (m, 2H), 7.25-7.23 (m, 1H), 6.89-6.87 (m, 1H), 4.33 (s, 2H), 4.28-4.11 (m, 1H), 3.77-3.41 (m, 6H), 3.24-3.08 (m, 2H), 1.70-1.55 (m, 5H), 1.51 (br d, J=8.0 Hz, 1H), 1.35 (br d, J=11.2 Hz, 9H), 1.11 (br s, 3H), 1.03-0.86 (m, 2H). ¹H NMR (400 MHz, CHLOROFORM-d) δ=10.41-10.22 (m, 1H), 8.53-8.43 (m, 1H), 7.84-7.66 (m, 3H), 7.39-7.30 (m, 2H), 7.07-7.05 (m, 1H), 5.25 (br d, J=9.2 Hz, 1H), 4.52-4.32 (m, 1H), 4.29 (s, 2H), 3.99-3.86 (m, 1H), 3.83-3.74 (m, 1H), 3.71-3.55 (m, 2H), 3.48-3.47 (m, 1H), 3.41-3.25 (m, 2H), 1.79-1.56 (m, 7H), 1.43 (d, J=6.4 Hz, 9H), 0.96 (br s, 5H). ¹⁹FNMR (377 MHz, CHLOROFORM-d) δ=−117.549.

### Step 2: Synthesis of Intermediate 1-3

To a mixture of I-860 (60 mg, 99.06 μmol, 1 eq) in DCM (1 mL) was added HCl/dioxane (2 M, 1 mL, 20.19 eq) and it was stirred at 25° C. for 3 hr. The mixture was concentrated under vacuum. The residue was used in the next step without purification. Intermediate 1-3 (60 mg, crude, HCl salt) as a white solid was obtained. LCMS (Method G): Rt=0.516 min, [M+H]⁺=506.4.

### Step 3: Synthesis of I-641

A mixture of Intermediate 1-3 (60 mg, 110.69 μmol, 1 eq, HCl salt), Intermediate 1-4 (32.25 mg, 110.69 μmol, 1 eq) and HOAt (15.07 mg, 110.69 μmol, 15.48 μL, 1 eq) in DMF (1 mL) was added EDCI (42.44 mg, 221.38 μmol, 2 eq) and NMM (55.98 mg, 553.46 μmol, 60.85 μL, 5 eq). The mixture was stirred at 25° C. for 1 hr. The mixture was poured into water (5 mL) and filtered, and the filter cake was concentrated under vacuum. The residue was purified by Prep-HPLC (column: Waters Xbridge 150*25 mm*5 um; mobile phase: [water (NH₄HCO₃)-ACN]; gradient: 32%-62% B over 15 min) and was dried by lyophilization. I-641 (24.24 mg, 30.73 μmol, 27.76% yield, 98.73% purity) was obtained as a white solid. LCMS (Method G): Rt=0.635 min, [M+H]⁺=779.4. SFC: Rt=1.694 min, 2.462 min. ¹H NMR (400 MHz, CHLOROFORM-d) δ=10.32-10.15 (m, 1H), 8.47 (br d, J=4.8 Hz, 1H), 7.95-7.67 (m, 4H), 7.44-7.30 (m, 4H), 7.25-7.17 (m, 1H), 7.07-7.03 (m, 1H), 5.14-4.94 (m, 1H), 4.78-4.62 (m, 1H), 4.29 (s, 3H), 4.08-3.27 (m, 8H), 3.24-2.98 (m, 2H), 2.84-2.54 (m, 1H), 2.07 (br d, J=11.6 Hz, 1H), 1.93-1.65 (m, 10H), 1.31-0.96 (m, 7H), 0.86-0.66 (m, 2H). ¹⁹F NMR (377 MHz, CHLOROFORM-d) δ=−117.555, −118.574, −119.391, −119.552.

### Step 4: Synthesis of Intermediate 1-4

To a solution of Intermediate 1-5 (0.92 g, 3.01 mmol, 1 eq) in THF (3 mL), H₂O (3 mL) and MeOH (3 mL) was added LiOH·H₂O (379.31 mg, 9.04 mmol, 3 eq). The mixture was stirred at 25° C. for 0.5 h. The mixture was concentrated under vacuum to remove THF and MeOH. Then the pH was adjusted to 3 by 1N HCl and extracted with EA (5 ml*3). The combined organic layers were washed with brine (5 ml*3), dried over anhydrous Na₂SO₄, filtered and concentrated under vacuum to give a crude product. The crude was used for the next step directly without purification. Intermediate 1-4 (750 mg, crude) was obtained as a yellow solid. ¹H NMR (400 MHz, DMSO-d₆) δ=14.10-11.22 (m, 1H), 7.72 (br s, 1H), 7.60 (br d, J=6.6 Hz, 1H), 7.26 (br s, 1H), 4.53-4.22 (m, 2H), 3.28-3.09 (m, 1H), 3.06-2.84 (m, 1H), 2.76-2.57 (m, 1H), 1.98-1.65 (m, 4H), 1.57-1.46 (m, 1H), 0.83-0.72 (m, 4H). ¹⁹F NMR (377 MHz, DMSO-d₆) δ=−117.322, −117.531.

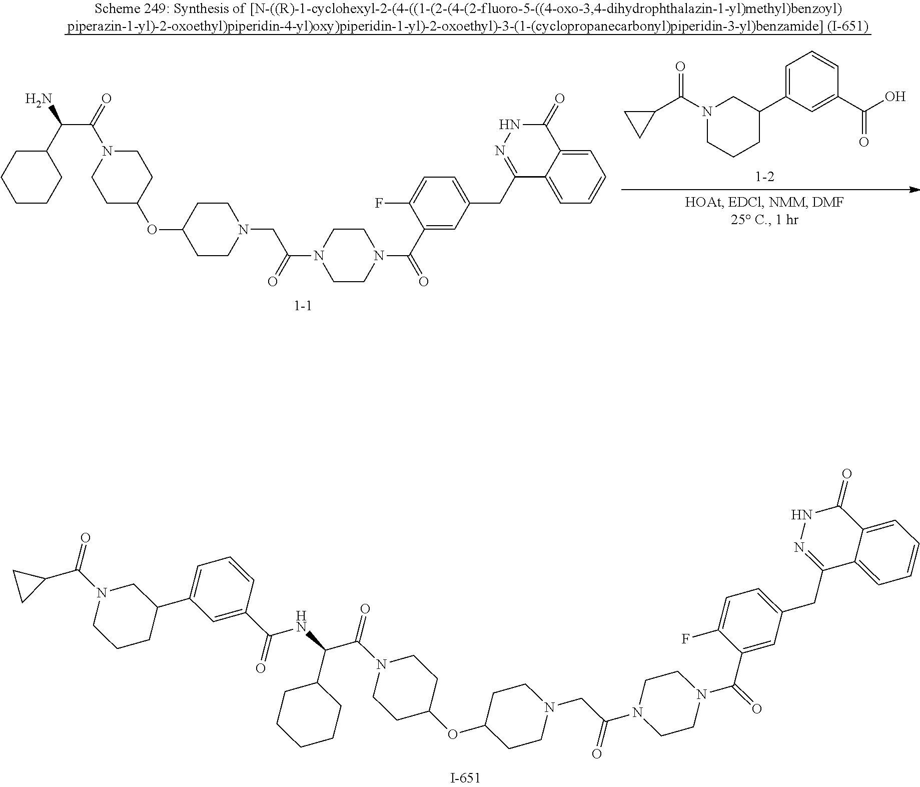 [SMILES: N[C@@H](C(=O)N1CCC(OC2CCN(CC(=O)N3CCN(C(=O)c4cc(Cc5n[nH]c(=O)c6ccccc56)ccc4F)CC3)CC2)CC1)C1CCCCC1 ; O=C(O)c1cccc(C2CCCN(C(=O)C3CC3)C2)c1 ; O=C(N[C@@H](C(=O)N1CCC(OC2CCN(CC(=O)N3CCN(C(=O)c4cc(Cc5n[nH]c(=O)c6ccccc56)ccc4F)CC3)CC2)CC1)C1CCCCC1)c1cccc(C2CCCN(C(=O)C3CC3)C2)c1]

To a solution of intermediate 1-2 (35.67 mg, 130.49 μmol, 1 eq) in DMF (1 mL) was added HOAt (17.76 mg, 130.49 μmol, 18.25 μL, 1 eq) and EDCI (75.05 mg, 391.47 μmol, 3 eq). Then intermediate 1-1 (100 mg, 130.49 μmol, 1 eq, HCl salt) and NMM (65.99 mg, 652.45 μmol, 71.73 μL, 5 eq) was added in to the mixture. The mixture was stirred at 25° C. for 0.5 hr. The reaction mixture was diluted with H₂O (2 mL) and extracted with EA (5 mL*3). The combined organic layers were washed with brine (5 mL*3), dried over Na₂SO₄, filtered and concentrated under reduced pressure to give a residue. The residue was purified by reversed phase HPLC (0.1% FA condition), the eluent was concentrated and lyophilized to give the desired product. I-651 (30.73 mg, 29.64 μmol, 22.72% yield, 99.468% purity, FA salt) was obtained as a white solid. LCMS (Method D): Retention time=0.416 min, (M+H)=985.4. SFC: Retention time=3.732 min, 4.646 min. ¹H NMR (400 MHz, METHANOL-d₄) δ=8.48 (s, 1H), 8.37 (d, J=7.6 Hz, 1H), 7.95 (d, J=7.2 Hz, 1H), 7.91-7.81 (m, 2H), 7.77 (d, J=13.2 Hz, 1H), 7.74-7.65 (m, 1H), 7.55-7.35 (m, 4H), 7.21-7.11 (m, 1H), 4.95-4.90 (m, 1H), 4.57 (d, J=10.4 Hz, 1H), 4.38 (s, 3H), 4.05 (d, J=12.0 Hz, 1H), 3.99-3.84 (m, 1H), 3.77 (d, J=17.6 Hz, 3H), 3.71-3.56 (m, 4H), 3.55-3.40 (m, 5H), 3.37-3.33 (m, 1H), 3.30-3.19 (m, 2H), 3.00-2.67 (m, 4H), 2.64-2.42 (m, 2H), 2.06 (d, J=12.8 Hz, 1H), 2.02-1.81 (m, 9H), 1.78 (d, J=11.2 Hz, 2H), 1.73-1.56 (m, 6H), 1.55-1.45 (m, 1H), 1.36-1.18 (m, 3H), 1.16-1.04 (m, 2H), 0.93-0.73 (m, 4H). ¹⁹F NMR (377 MHz, METHANOL-d₄), δ=−120.749.

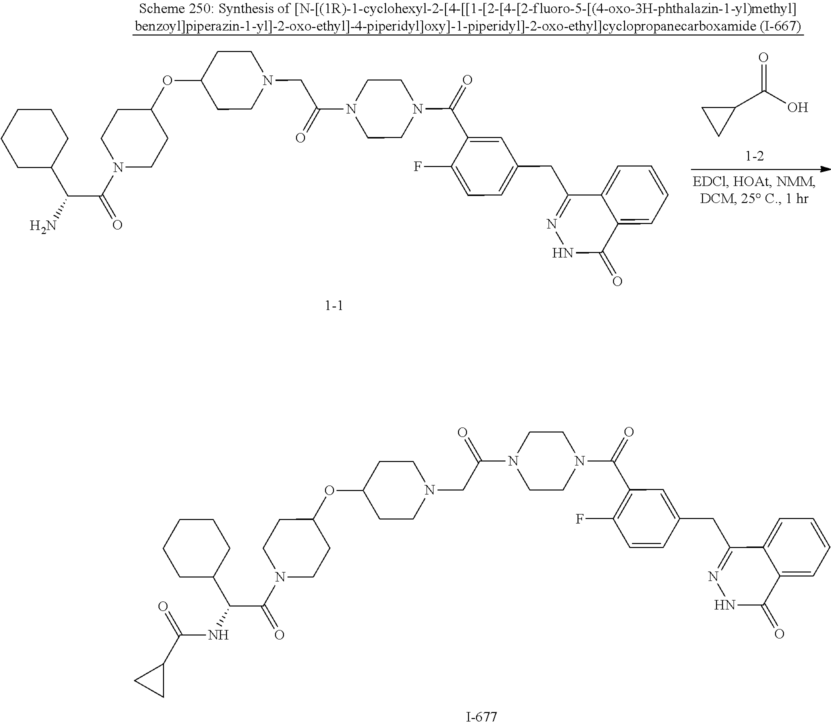 [SMILES: N[C@@H](C(=O)N1CCC(OC2CCN(CC(=O)N3CCN(C(=O)c4cc(Cc5n[nH]c(=O)c6ccccc56)ccc4F)CC3)CC2)CC1)C1CCCCC1 ; O=C(O)C1CC1 ; O=C(N[C@@H](C(=O)N1CCC(OC2CCN(CC(=O)N3CCN(C(=O)c4cc(Cc5n[nH]c(=O)c6ccccc56)ccc4F)CC3)CC2)CC1)C1CCCCC1)C1CC1]

To a solution of intermediate 1-2 (78.79 mg, 411.03 μmol, 3 eq) in DCM (2 mL) was added EDCI (78.79 mg, 411.03 μmol, 3 eq), NMM (69.29 mg, 685.04 μmol, 75.32 μL, 5 eq) and HOAt (18.65 mg, 137.01 μmol, 19.17 μL, 1 eq) at 25° C., then the mixture was stirred at 25° C. for 0.5 hr. Intermediate 1-1 (100 mg, 137.01 μmol, 1 eq) was added into the mixture and stirred at 25° C. for 0.5 hr. The reaction mixture was diluted with water (30 mL) and extracted with EA (10 mL×3). The combined organic layers were washed with brine (10 mL×3) and dried over sodium sulfate, filtered and concentrated under reduced pressure to give a residue. The residue was purified by reversed phase HPLC (neutral condition) and the eluent was concentrated under reduced pressure to remove ACN and then lyophilized to give I-667 (46.92 mg, 56.73 μmol, 41.41% yield, 96.481% purity), which was obtained as a white solid. LCMS (Method D): Rt: 0.360 min, [M+H]⁺=798.3. ¹H NMR (400 MHz, METHANOL-d4) δ=8.36 (d, J=7.6 Hz, 1H), 7.98-7.91 (m, 1H), 7.90-7.78 (m, 2H), 7.48 (s, 1H), 7.42-7.35 (m, 1H), 7.16 (s, 1H), 4.72 (d, J=8.0 Hz, 1H), 4.38 (s, 2H), 4.04-3.88 (m, 1H), 3.81 (d, J=15.6 Hz, 2H), 3.76-3.61 (m, 4H), 3.60-3.44 (m, 4H), 3.40 (d, J=8.8 Hz, 1H), 3.26-3.11 (m, 3H), 2.76 (s, 2H), 2.31-2.20 (m, 2H), 1.94-1.65 (m, 11H), 1.64-1.42 (m, 5H), 1.33-1.17 (m, 3H), 1.16-0.97 (m, 3H), 0.89-0.79 (m, 2H), 0.78-0.68 (m, 2H). ¹⁹F NMR (376 MHz, METHANOL-d4) δ=−120.693 (s). SFC: Rt: 1.535 min.

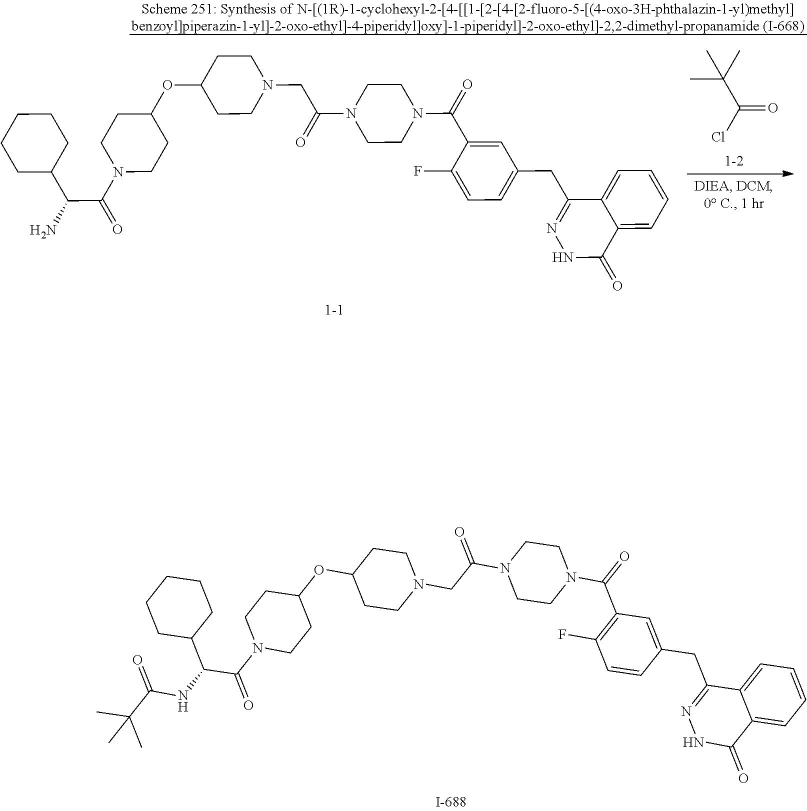

To a solution of intermediate 1-1 (70 mg, 95.91 μmol, 1 eq) in DCM (1 mL) was added DIEA (61.98 mg, 479.53 μmol, 83.53 μL, 5 eq) and intermediate 1-2 (15.03 mg, 124.68 μmol, 15.34 μL, 1.3 eq). The mixture was stirred at 0° C. for 1 hr. The reaction mixture was diluted with water (30 mL) and extracted with EA (10 mL×3). The combined organic layers were washed with brine (10 mL×3) and dried over sodium sulfate, filtered and concentrated under reduced pressure to give a residue. The crude product was purified by reversed-phase HPLC (column: CD02-Waters Xbidge BEH C18 150*25*10 um; mobile phase: [water (NH₄HCO₃)-ACN]; gradient: 30%-60% B over 10 min) and the eluent was concentrated under reduced pressure to remove ACN and then lyophilized to give I-668 (35.44 mg, 43.54 μmol, 45.40% yield, 100% purity) as a white solid. LCMS (Method D): Rt: 0.375 min, [M+H]⁺=814.5. ¹H NMR (400 MHz, METHANOL-d4) δ=8.36 (d, J=7.6 Hz, 1H), 7.98-7.91 (m, 1H), 7.90-7.78 (m, 2H), 7.48 (s, 1H), 7.38 (s, 1H), 7.16 (s, 1H), 4.75 (d, J=7.6 Hz, 1H), 4.38 (s, 2H), 4.02-3.64 (m, 7H), 3.63-3.32 (m, 6H), 3.28-3.11 (m, 3H), 2.76 (s, 2H), 2.26 (d, J=11.2 Hz, 2H), 1.93-1.69 (m, 8H), 1.69-1.42 (m, 6H), 1.34-1.09 (m, 12H), 1.08-0.93 (m, 2H). ¹⁹F NMR (376 MHz, METHANOL-d4) δ=−120.690. SFC: 0.648 min.

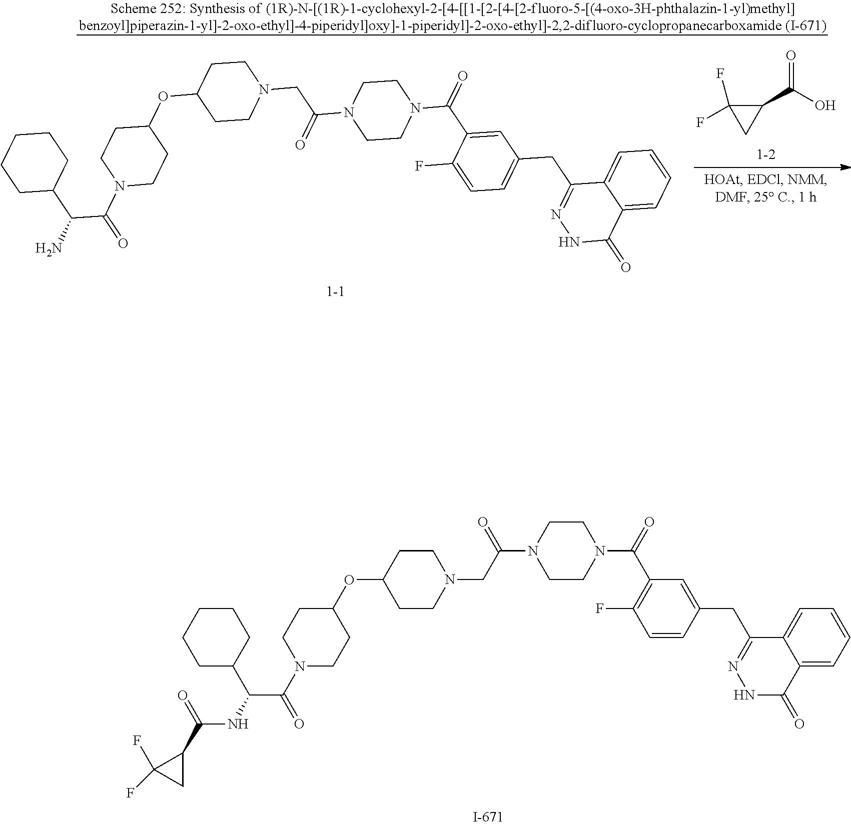 [SMILES: N[C@@H](C(=O)N1CCC(OC2CCN(CC(=O)N3CCN(C(=O)c4cc(Cc5n[nH]c(=O)c6ccccc56)ccc4F)CC3)CC2)CC1)C1CCCCC1 ; O=C(O)[C@H]1CC1(F)F ; O=C(N[C@@H](C(=O)N1CCC(OC2CCN(CC(=O)N3CCN(C(=O)c4cc(Cc5n[nH]c(=O)c6ccccc56)ccc4F)CC3)CC2)CC1)C1CCCCC1)[C@H]1CC1(F)F]

To a solution of intermediate 1-1 (30 mg, 39.15 μmol, 1 eq, HCl salt) and intermediate 1-2 (4.78 mg, 39.15 μmol, 1 eq) in DMF (0.3 mL) was added EDCI (22.51 mg, 117.44 μmol, 3 eq), HOAt (5.33 mg, 39.15 μmol, 5.48 μL, 1 eq) and NMM (19.80 mg, 195.74 μmol, 21.52 μL, 5 eq). The mixture was stirred at 25° C. for 1 hr. The reaction mixture was concentrated under reduced pressure to give a residue. The crude product was purified by reversed phase HPLC (0.1% TFA condition) and lyophilized to give a residue. I-671 (29.88 mg, 31.28 μmol, 79.91% yield, 99.241% purity, TFA salt) was obtained as a white solid. LCMS (Method D): Rt=0.357 min, [M+H]⁺=834.9. SFC: Retention time: 1.222 min. ¹H NMR (400 MHz, METHANOL-d4) δ=8.38 (d, J=7.6 Hz, 1H), 8.00-7.94 (m, 1H), 7.93-7.83 (m, 2H), 7.55-7.48 (m, 1H), 7.40-7.32 (m, 1H), 7.19 (t, J=8.8 Hz, 1H), 4.74 (d, J=7.6 Hz, 1H), 4.40 (s, 2H), 4.29 (s, 1H), 4.22 (s, 1H), 3.96 (s, 2H), 3.87-3.63 (m, 6H), 3.54 (d, J=4.0 Hz, 3H), 3.46 (d, J=12.0 Hz, 2H), 3.41-3.33 (m, 4H), 3.20-3.05 (m, 1H), 2.71-2.57 (m, 1H), 2.29-2.10 (m, 1H), 2.06 (s, 2H), 2.01-1.82 (m, 4H), 1.81-1.65 (m, 6H), 1.64-1.44 (m, 3H), 1.33-1.15 (m, 3H), 1.12-0.99 (m, 2H). ¹⁹F NMR (400 MHz, METHANOL-d4) δ=−76.774, −120.449, −127.076, −142.568.

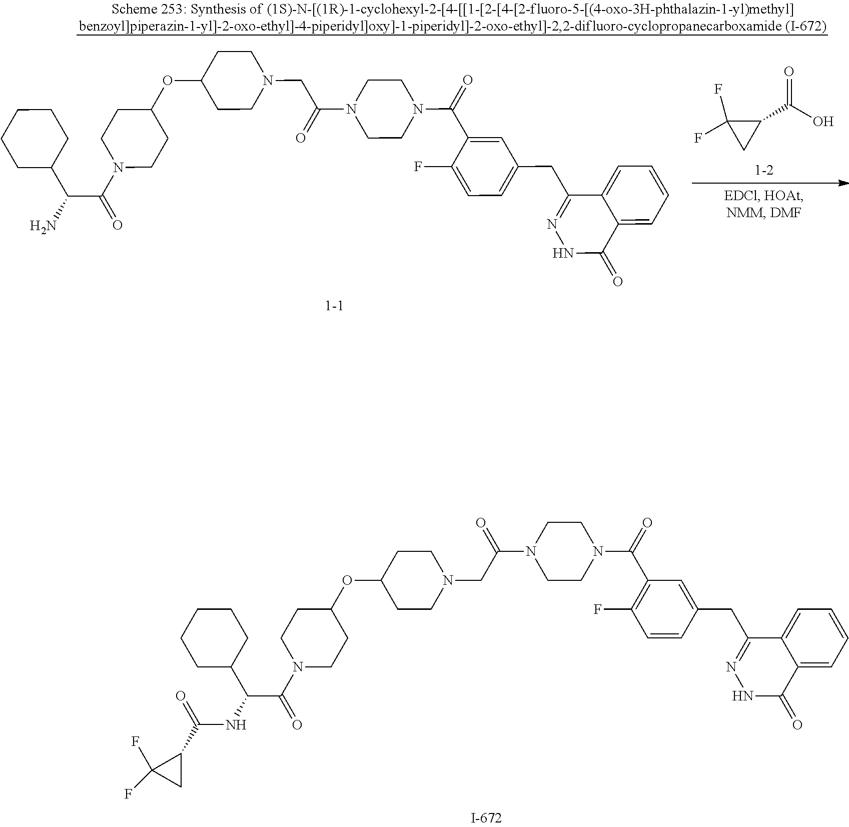 [SMILES: N[C@@H](C(=O)N1CCC(OC2CCN(CC(=O)N3CCN(C(=O)c4cc(Cc5n[nH]c(=O)c6ccccc56)ccc4F)CC3)CC2)CC1)C1CCCCC1 ; O=C(O)[C@@H]1CC1(F)F ; O=C(N[C@@H](C(=O)N1CCC(OC2CCN(CC(=O)N3CCN(C(=O)c4cc(Cc5n[nH]c(=O)c6ccccc56)ccc4F)CC3)CC2)CC1)C1CCCCC1)[C@@H]1CC1(F)F]

To a solution of intermediate 1-2 (4.78 mg, 39.15 μmol, 1 eq) in DMF (0.5 mL) was added HOAt (5.33 mg, 39.15 μmol, 5.48 μL, 1 eq), EDCI (22.51 mg, 117.44 μmol, 3 eq), NMM (19.80 mg, 195.74 μmol, 21.52 μL, 5 eq) and intermediate 1-1 (30 mg, 39.15 μmol, 1 eq, HCl salt). The mixture was stirred at 25° C. for 1 hr. The reaction liquid was concentrated under vacuum to obtain the crude product. The residue was purified by prep-HPLC (column: CD01-Phenomenex luna C18 150*25*10 um; mobile phase: [water (FA)-ACN]; gradient: 13%-43% B over 10 min), which was concentrated and lyophilized to afford I-672 (23.45 mg, 26.53 μmol, 67.76% yield, 99.536% purity, FA salt) as a white solid. LCMS (Method D): Rt=0.362 min, [M+H]⁺=834.3. ¹H NMR (400 MHz, METHANOL-d4) δ=8.54-8.46 (m, 1H), 8.37 (d, J=7.6 Hz, 1H), 7.98-7.92 (m, 1H), 7.91-7.81 (m, 2H), 7.54-7.46 (m, 1H), 7.38 (t, J=5.2 Hz, 1H), 7.17 (t, J=9.2 Hz, 1H), 4.75 (d, J=8.0 Hz, 1H), 4.39 (s, 2H), 4.08-3.89 (m, 1H), 3.87-3.72 (m, 4H), 3.70-3.56 (m, 4H), 3.52 (d, J=4.0 Hz, 4H), 3.40-3.33 (m, 2H), 3.29-3.11 (m, 1H), 3.05-2.87 (m, 2H), 2.72-2.46 (m, 3H), 2.08-1.87 (m, 4H), 1.83-1.65 (m, 9H), 1.63-1.40 (m, 3H), 1.36-1.16 (m, 3H), 1.11-0.96 (m, 2H). ¹⁹F NMR (376 MHz, METHANOL-d4) δ=−120.61 (s), −120.78-−127.61 (d), −142.92-−143.49 (m). SFC: Rt=0.963 min.

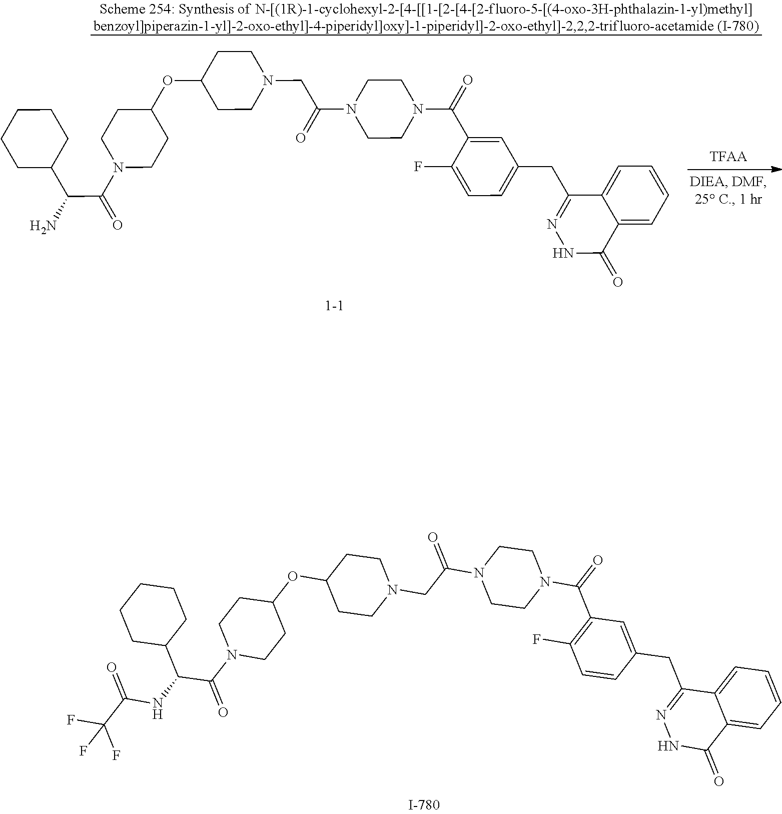 [SMILES: N[C@@H](C(=O)N1CCC(OC2CCN(CC(=O)N3CCN(C(=O)c4cc(Cc5n[nH]c(=O)c6ccccc56)ccc4F)CC3)CC2)CC1)C1CCCCC1 ; O=C(CN1CCC(OC2CCN(C(=O)[C@H](NC(=O)C(F)(F)F)C3CCCCC3)CC2)CC1)N1CCN(C(=O)c2cc(Cc3n[nH]c(=O)c4ccccc34)ccc2F)CC1]

To a solution of intermediate 1-1 (100 mg, 130.49 μmol, 1 eq, HCl salt) in DMF (1 mL) was added DIEA (50.59 mg, 391.47 μmol, 68.19 μL, 3 eq) and TFAA (82.22 mg, 391.47 μmol, 54.41 μL, 3 eq). The mixture was stirred at 25° C. for 1 hr. The mixture was washed with water (2 mL) and extracted with EA (3 mL*3). The combined organic layers were dried over sodium sulfate, filtered and concentrated under reduced pressure to give a residue. The residue was purified by reversed phase HPLC (0.1% TFA condition), the eluent was concentrated under reduced pressure to remove ACN and lyophilized to give product. I-780 (67.2 mg, 70.68 μmol, 54.17% yield, 98.865% purity, TFA salt) was obtained as a white solid. LCMS (Method D): Rt=0.362 min, [M+H]⁺=826.7. SFC: Rt=1.454 min. ¹H NMR (400 MHz, METHANOL-d4) δ=8.37 (d, J=7.6 Hz, 1H), 7.94 (d, J=4.0 Hz, 1H), 7.90-7.81 (m, 2H), 7.50 (s, 1H), 7.37-7.35 (m, 1H), 7.17-7.15 (m, 1H), 4.72 (d, J=8.8 Hz, 1H), 4.39 (s, 2H), 4.04-3.94 (m, 1H), 3.80 (d, J=4.4 Hz, 2H), 3.76 (d, J=4.4 Hz, 2H), 3.70 (d, J=3.2 Hz, 2H), 3.61 (s, 1H), 3.53 (d, J=4.4 Hz, 2H), 3.46 (s, 2H), 3.26-3.20 (m, 1H), 3.16-3.05 (m, 2H), 2.98-2.68 (m, 2H), 1.98 (d, J=1.6 Hz, 3H), 1.90-1.82 (m, 3H), 1.78 (d, J=11.2 Hz, 4H), 1.71-1.56 (m, 4H), 1.54-1.45 (m, 1H), 1.37-1.13 (m, 4H), 1.09-0.99 (m, 2H). ¹⁹F NMR (377 MHz, METHANOL-d4) δ=−76.74, −76.93, −120.74.

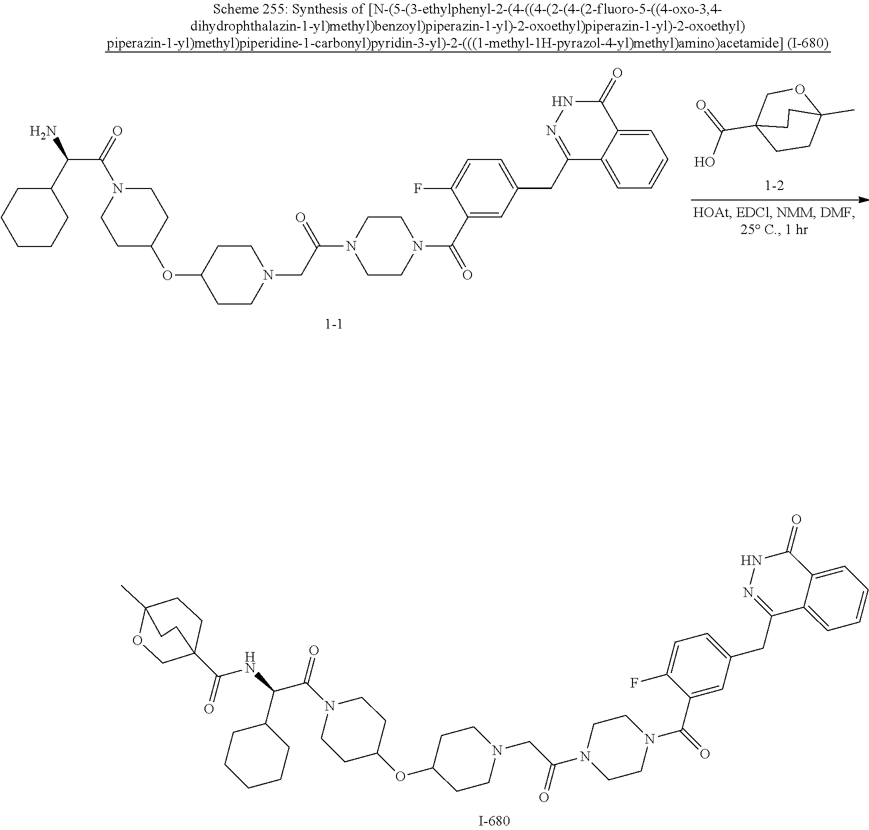

To a solution of intermediate 1-2 (17.77 mg, 104.39 μmol, 1 eq) in DMF (1 mL) was added HOAt (14.21 mg, 104.39 μmol, 14.60 μL, 1 eq) and EDCI (60.04 mg, 313.18 μmol, 3 eq). Then intermediate 1-1 (80 mg, 104.39 μmol, 1 eq, HCl salt) and NMM (52.79 mg, 521.96 μmol, 57.39 μL, 5 eq) was added into the mixture. The mixture was stirred at 25° C. for 0.5 hr. The reaction mixture was diluted with H₂O (5 mL) and extracted with EA (5 mL*3). The combined organic layers were dried over Na₂SO₄, filtered and concentrated under reduced pressure to give a residue. The crude product was purified by reversed phase HPLC (0.1% FA condition), the eluent was concentrated and lyophilized to give the desired product. I-680 (25.74 mg, 27.16 μmol, 26.01% yield, 97.919% purity, FA salt) was obtained as a white solid. LCMS (Method D): Retention time: 0.381 min, [M+H]⁺=882.3. SFC: Retention time: 1.456 min. ¹H NMR (400 MHz, METHANOL-d4) δ=8.43 (s, 2H), 8.37 (d, J=7.6 Hz, 1H), 7.98-7.92 (m, 1H), 7.91-7.81 (m, 2H), 7.50 (d, J=3.2 Hz, 1H), 7.41-7.33 (m, 1H), 7.28 (d, J=6.0 Hz, 1H), 7.20-7.13 (m, 1H), 4.75-4.67 (m, 1H), 4.39 (s, 2H), 4.01-3.88 (m, 3H), 3.86-3.67 (m, 8H), 3.65-3.35 (m, 6H), 3.18-3.05 (m, 2H), 2.92-2.76 (m, 2H), 2.05-1.88 (m, 7H), 1.87-1.63 (m, 13H), 1.60 (d, J=12.0 Hz, 3H), 1.32-1.15 (m, 3H), 1.08 (s, 3H), 1.03-0.90 (m, 2H). ¹⁹FNMR (376 MHz, METHANOL-d4) δ=−120.75.

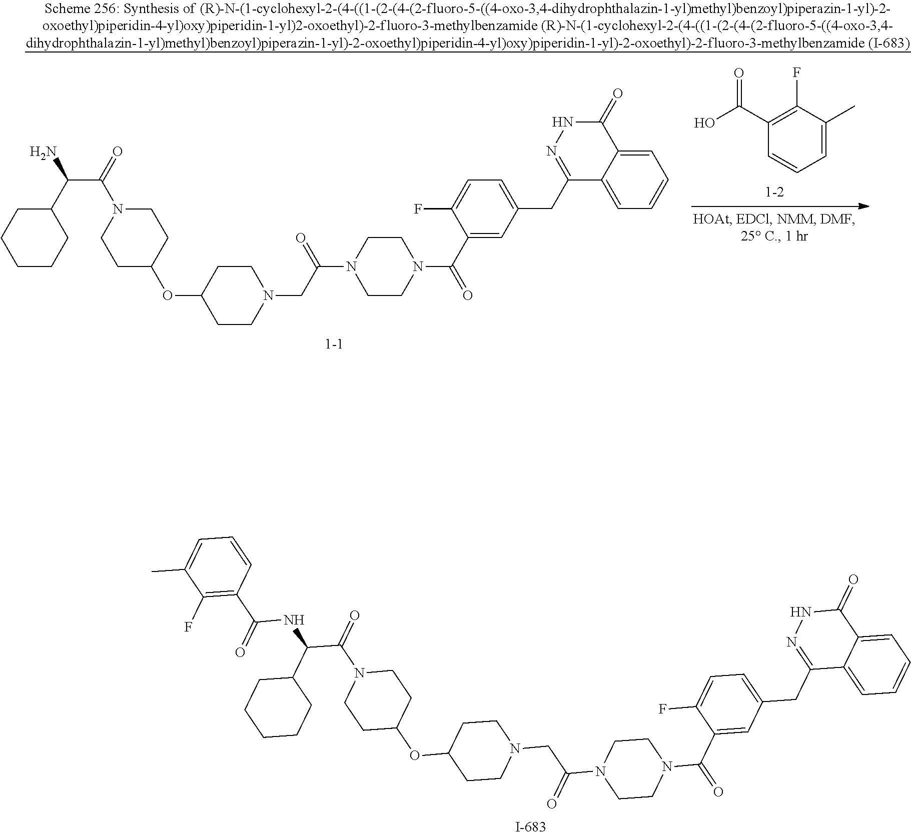 [SMILES: N[C@@H](C(=O)N1CCC(OC2CCN(CC(=O)N3CCN(C(=O)c4cc(Cc5n[nH]c(=O)c6ccccc56)ccc4F)CC3)CC2)CC1)C1CCCCC1 ; Cc1cccc(C(=O)O)c1F ; Cc1cccc(C(=O)N[C@@H](C(=O)N2CCC(OC3CCN(CC(=O)N4CCN(C(=O)c5cc(Cc6n[nH]c(=O)c7ccccc67)ccc5F)CC4)CC3)CC2)C2CCCCC2)c1F]

To a solution of intermediate 1-2 (160.91 mg, 1.04 mmol, 1 eq) in DMF (8 mL) was added HOAt (142.09 mg, 1.04 mmol, 146.03 μL, 1 eq), EDCI (600.36 mg, 3.13 mmol, 3 eq), NMM (527.95 mg, 5.22 mmol, 573.86 μL, 5 eq) and intermediate 1-1 (800 mg, 1.04 mmol, 1 eq, HCl salt). The mixture was stirred at 25° C. for 1 hr. The mixture was washed with water (10 mL) and extracted with EA (10 mL*3). The combined organic layers were dried over sodium sulfate, filtered and concentrated under reduced pressure to give a residue. The residue was purified by prep-HPLC (column: CD05-Phenomenex luna C18 150*40*10 um; mobile phase: [water(FA)-ACN]; gradient: 16%-46% B over 10 min), the eluent was concentrated under reduced pressure to remove ACN and lyophilized to give I-683 (369.41 mg, 400.75 μmol, 38.39% yield, 98.94% purity, FA salt) as a white solid. LCMS (Method D): Rt=0.387 min, [M+H]⁺=866.4. SFC: Rt: 1.267 min. ¹H NMR (400 MHz, METHANOL-d4) δ=8.42 (d, J=6.4 Hz, 1H), 8.37 (d, J=7.6 Hz, 1H), 7.95 (d, J=6.4 Hz, 1H), 7.91-7.80 (m, 2H), 7.55-7.48 (m, 2H), 7.44-7.33 (m, 2H), 7.21-7.12 (m, 2H), 4.97 (d, J=7.6 Hz, 1H), 4.39 (s, 2H), 4.07-3.97 (m, 1H), 3.95-3.69 (m, 8H), 3.62 (s, 2H), 3.55-3.44 (m, 3H), 3.38-3.34 (m, 1H), 3.25 (s, 1H), 3.16-3.04 (m, 2H), 2.92-2.70 (m, 2H), 2.32 (s, 3H), 2.04-1.91 (m, 3H), 1.88-1.74 (m, 7H), 1.69 (d, J=9.6 Hz, 3H), 1.58-1.47 (m, 1H), 1.36-1.20 (m, 3H), 1.19-1.09 (m, 2H). ¹⁹F NMR (376 MHz, METHANOL-d4) δ=−120.4 (s), −120.8 (s).

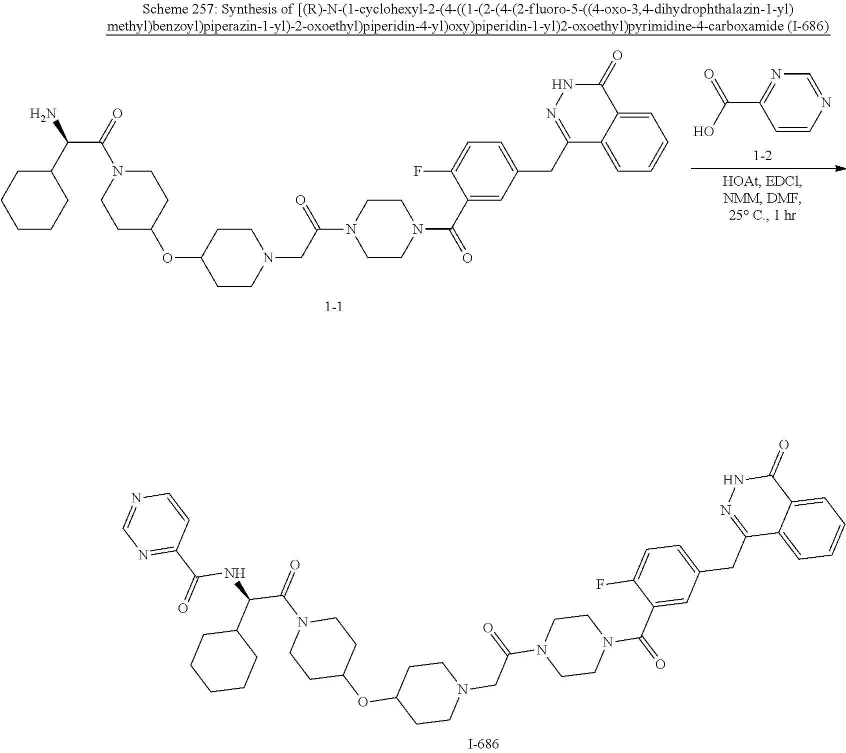

To a solution of intermediate 1-2 (9.72 mg, 78.29 μmol, 1 eq) in DMF (0.5 mL) was added HOAt (10.66 mg, 78.29 μmol, 10.95 μL, 1 eq) and EDCI (45.03 mg, 234.88 μmol, 3 eq). Then intermediate 1-1 (60 mg, 78.29 μmol, 1 eq, HCl salt) and NMM (39.60 mg, 391.47 μmol, 43.04 μL, 5 eq) was added into the mixture. The mixture was stirred at 25° C. for 0.5 hr. The reaction mixture was diluted with H₂O (2 mL) and extracted with EA (5 mL*3). The combined organic layers were washed with brine (5 mL*3), dried over Na₂SO₄, filtered and concentrated under reduced pressure to give a residue. The residue was purified by prep-HPLC (neutral condition), the eluent was concentrated and lyophilized to give the desired product. I-686 (7.52 mg, 8.95 μmol, 11.43% yield, 99.485% purity) was obtained as a white solid. LCMS (Method D): Retention time: 0.348 min, [M+H]⁺=836.4. SFC: Retention time: 1.914 min. ¹H NMR (400 MHz, METHANOL-d4) δ=9.30 (s, 1H), 9.03 (d, J=5.2 Hz, 1H), 8.37 (d, J=7.8 Hz, 1H), 8.08 (d, J=4.4 Hz, 1H), 7.95 (d, J=7.8 Hz, 1H), 7.90-7.79 (m, 2H), 7.53-7.35 (m, 2H), 7.22-7.10 (m, 1H), 5.00 (d, J=7.2 Hz, 1H), 4.39 (s, 2H), 4.05-3.84 (m, 2H), 3.82-3.63 (m, 6H), 3.61-3.41 (m, 6H), 2.92-2.77 (m, 2H), 2.52-2.26 (m, 2H), 1.95-1.82 (m, 5H), 1.80-1.73 (m, 4H), 1.70-1.58 (m, 4H), 1.37-1.24 (m, 4H), 1.23-1.06 (m, 4H). ¹⁹FNMR (376 MHz, METHANOL-d4) δ=−120.67.

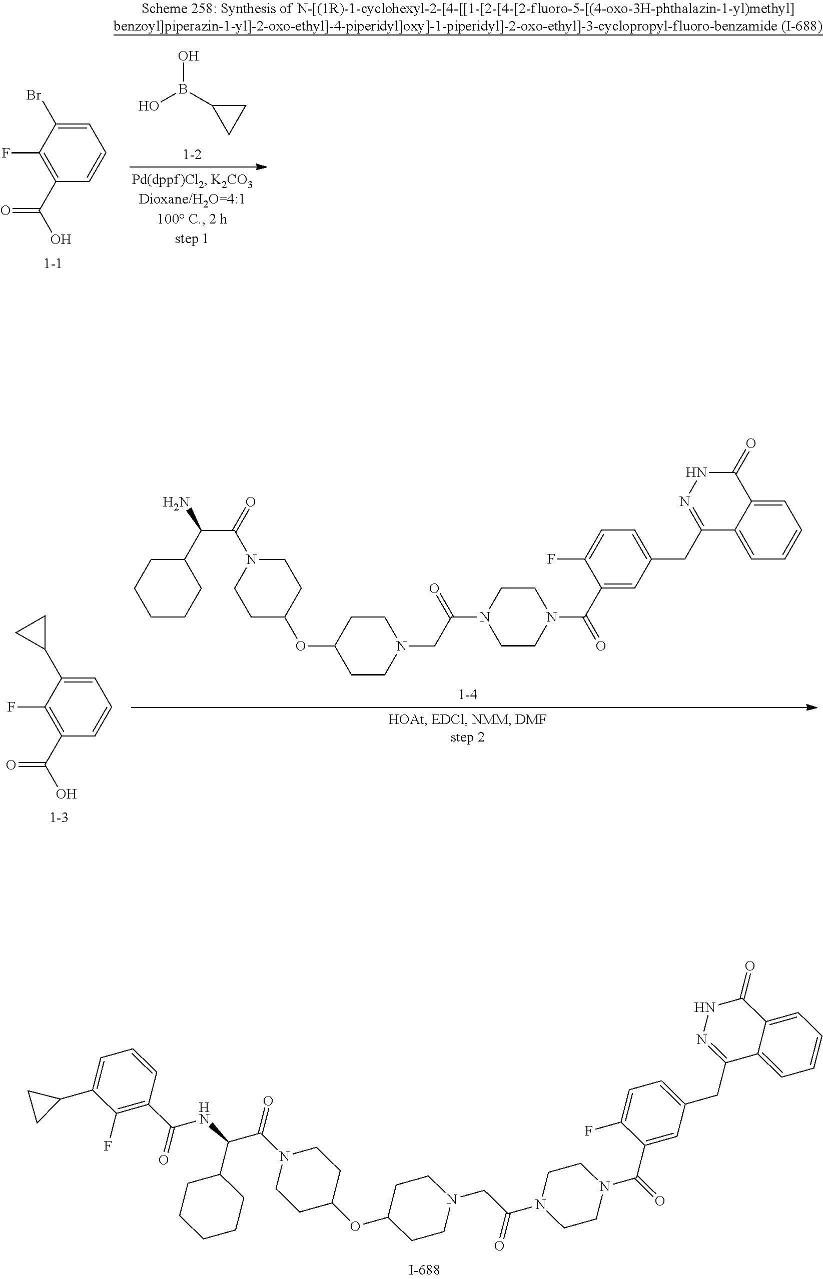 [SMILES: O=C(O)c1cccc(Br)c1F ; OB(O)C1CC1 ; O=C(O)c1cccc(C2CC2)c1F ; N[C@@H](C(=O)N1CCC(OC2CCN(CC(=O)N3CCN(C(=O)c4cc(Cc5n[nH]c(=O)c6ccccc56)ccc4F)CC3)CC2)CC1)C1CCCCC1 ; O=C(N[C@@H](C(=O)N1CCC(OC2CCN(CC(=O)N3CCN(C(=O)c4cc(Cc5n[nH]c(=O)c6ccccc56)ccc4F)CC3)CC2)CC1)C1CCCCC1)c1cccc(C2CC2)c1F]

### Step 1: Synthesis of Intermediate 1-3

To a solution of Intermediate 1-1 (2 g, 9.13 mmol, 1 eq) in dioxane (16 mL) and H₂O (4 mL) was added Intermediate 1-2 (1.18 g, 13.70 mmol, 1.5 eq), Pd(dppf)Cl₂ (668.20 mg, 913.21 μmol, 0.1 eq) and K₂CO₃ (2.52 g, 18.26 mmol, 2 eq) and the mixture was degassed and purged with N₂ 3 times, and then the mixture was stirred at 100° C. for 2 hr under N₂ atmosphere. The reaction mixture was quenched by H₂O (20 mL), and extracted with EA (10 mL*3). The combined organic layers were washed with H₂O (10 mL*2), the aqueous layer was adjusted to pH=3 by 1N HCl, extracted with EA (10 mL*3), and the combined organic layer was dried over anhydrous Na₂SO₄, filtered and concentrated under reduced pressure to give a residue. The residue was purified by flash silica gel chromatography (ISCO; 20 g SepaFlash Silica Flash Column, Eluent of 0˜20% Ethyl acetate/Petroleum ether gradient @100 mL/min) and concentrated under reduced pressure. Intermediate 1-3 (0.85 g, 4.72 mmol, 51.66% yield) was obtained as a white solid. LCMS (Method C): Rt=0.328 min, [M−H]⁺=179.1. ¹H NMR (400 MHz, DMSO-d₆) δ=13.15 (br s, 1H), 7.61 (dt, J=2.0, 7.2 Hz, 1H), 7.23-7.05 (m, 2H), 2.23-1.93 (m, 1H), 1.04-0.95 (m, 2H), 0.77-0.70 (m, 2H). ¹⁹F NMR (377 MHz, DMSO-d₆) δ=−118.579.

### Step 2: Synthesis of I-688

To a solution of Intermediate 1-3 (0.35 g, 1.94 mmol, 1 eq) in DMF (5 mL) was added Intermediate 1-4 (1.49 g, 1.94 mmol, 1 eq, HCl salt), HOAt (264.40 mg, 1.94 mmol, 271.74 μL, 1 eq), EDCI (744.78 mg, 3.89 mmol, 2 eq) and NMM (982.42 mg, 9.71 mmol, 1.07 mL, 5 eq). The mixture was stirred at 25° C. for 1 h. The mixture was poured into H₂O (10 mL), filtered and the filter cake was washed with H₂O (2 mL*3) and concentrated under vacuum to give a crude product. The crude was purified by Prep-HPLC (column: Kromasil Eternity XT 250*80 mm*10 um; mobile phase: [water (NH₃H₂O)-ACN]; gradient: 40%-70% B over 30 min) and (column: Phenomenex luna C18 150*40 mm*15 um; mobile phase: [water (FA)-ACN]; gradient: 25%-55% B over 15 min). I-688 (690.88 mg, 734.43 μmol, 37.81% yield, 99.72% purity, FA salt) was obtained as a white solid. LCMS (Method G): Rt=0.713 min, [M+H]⁺=892.5. SFC: Rt=1.421 min, ee %=94.932%. ¹H NMR (400 MHz, METHANOL-d₄) δ=8.60-8.51 (m, 1H), 8.38 (br d, J=7.2 Hz, 1H), 8.02-7.78 (m, 3H), 7.57-7.45 (m, 2H), 7.39 (br s, 1H), 7.22-7.10 (m, 3H), 5.00 (br d, J=7.6 Hz, 1H), 4.40 (s, 2H), 4.10-3.38 (m, 14H), 3.31-3.24 (m, 1H), 2.87 (br s, 2H), 2.43 (br d, J=3.6 Hz, 2H), 2.19-2.09 (m, 1H), 1.99-1.48 (m, 15H), 1.33-1.09 (m, 5H), 1.08-1.00 (m, 2H), 0.79-0.71 (m, 2H). ¹⁹F NMR (377 MHz, METHANOL-d₄) δ=−120.702, −123.170.

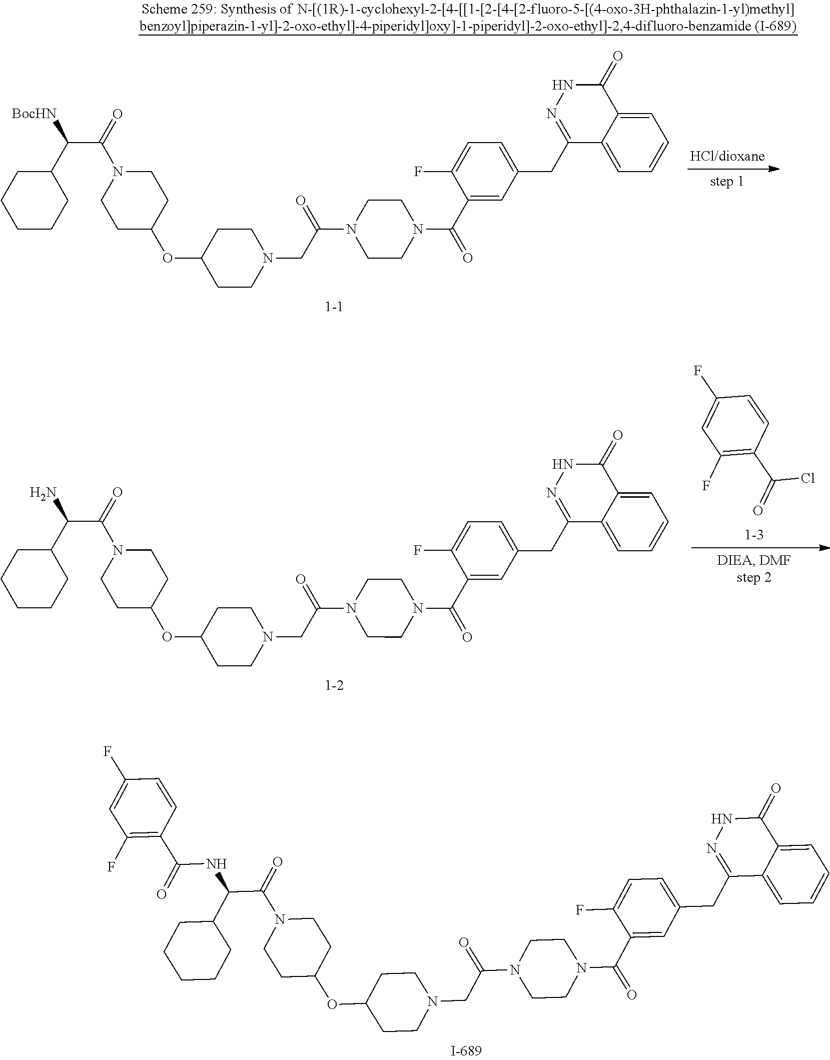 [SMILES: CC(C)(C)OC(=O)N[C@@H](C(=O)N1CCC(OC2CCN(CC(=O)N3CCN(C(=O)c4cc(Cc5n[nH]c(=O)c6ccccc56)ccc4F)CC3)CC2)CC1)C1CCCCC1 ; N[C@@H](C(=O)N1CCC(OC2CCN(CC(=O)N3CCN(C(=O)c4cc(Cc5n[nH]c(=O)c6ccccc56)ccc4F)CC3)CC2)CC1)C1CCCCC1 ; O=C(Cl)c1ccc(F)cc1F ; O=C(N[C@@H](C(=O)N1CCC(OC2CCN(CC(=O)N3CCN(C(=O)c4cc(Cc5n[nH]c(=O)c6ccccc56)ccc4F)CC3)CC2)CC1)C1CCCCC1)c1ccc(F)cc1F]

### Step 1: Synthesis of Intermediate 1-2

To a solution of Intermediate 1-1 (4 g, 4.82 mmol, 1 eq) in DCM (8 mL) was added HCl/dioxane (2 M, 32 mL, 13.28 eq). The mixture was stirred at 25° C. for 20 min. The mixture was concentrated under vacuum to give a crude product. The crude was used for the next step directly without purification. Intermediate 1-2 (4 g, crude, HCl salt) was obtained as a yellow solid. LCMS (Method G): Rt=0.559 min, [M+H]⁺=730.6.

### Step 2: Synthesis of I-689

To a solution of Intermediate 1-2 (1 g, 1.30 mmol, 1 eq, HCl salt) and DIEA (505.95 mg, 3.91 mmol, 681.87 μL, 3 eq) in DMF (10 mL) was added Intermediate 1-3 (276.45 mg, 1.57 mmol, 192.38 μL, 1.2 eq) at 0° C. Then the mixture was warmed to 25° C. and stirred at 25° C. for 1 h. The mixture was poured onto H₂O (10 mL), filtered and the filter cake was washed with H₂O (2 mL*3) and then dried under vacuum to give a crude product. The crude was purified by Prep-HPLC (column: Kromasil Eternity XT 250*80 mm*10 um; mobile phase: [water (NH₃H2O)-ACN]; gradient: 35%-65% B over 30 min). I-689 (569.83 mg, 648.25 μmol, 49.68% yield, 98.97% purity) was obtained as a white solid. LCMS (Method G): Rt=0.669 min, [M+H]⁺=870.5. SFC: Rt=1.617 min. ¹H NMR (400 MHz, METHANOL-d₄) δ=8.36 (br d, J=7.6 Hz, 1H), 7.94 (br d, J=8.0 Hz, 1H), 7.88-7.75 (m, 3H), 7.48 (br s, 1H), 7.37 (br d, J=5.2 Hz, 1H), 7.18-7.05 (m, 3H), 4.96 (br d, J=7.6 Hz, 1H), 4.38 (s, 2H), 4.06-3.94 (m, 1H), 3.93-3.37 (m, 12H), 3.28-3.20 (m, 2H), 2.85-2.71 (m, 2H), 2.37-2.19 (m, 2H), 1.97-1.49 (m, 15H), 1.30-1.07 (m, 5H). ¹⁹F NMR (377 MHz, METHANOL-d₄) δ=−106.827, −110.958, −120.780.

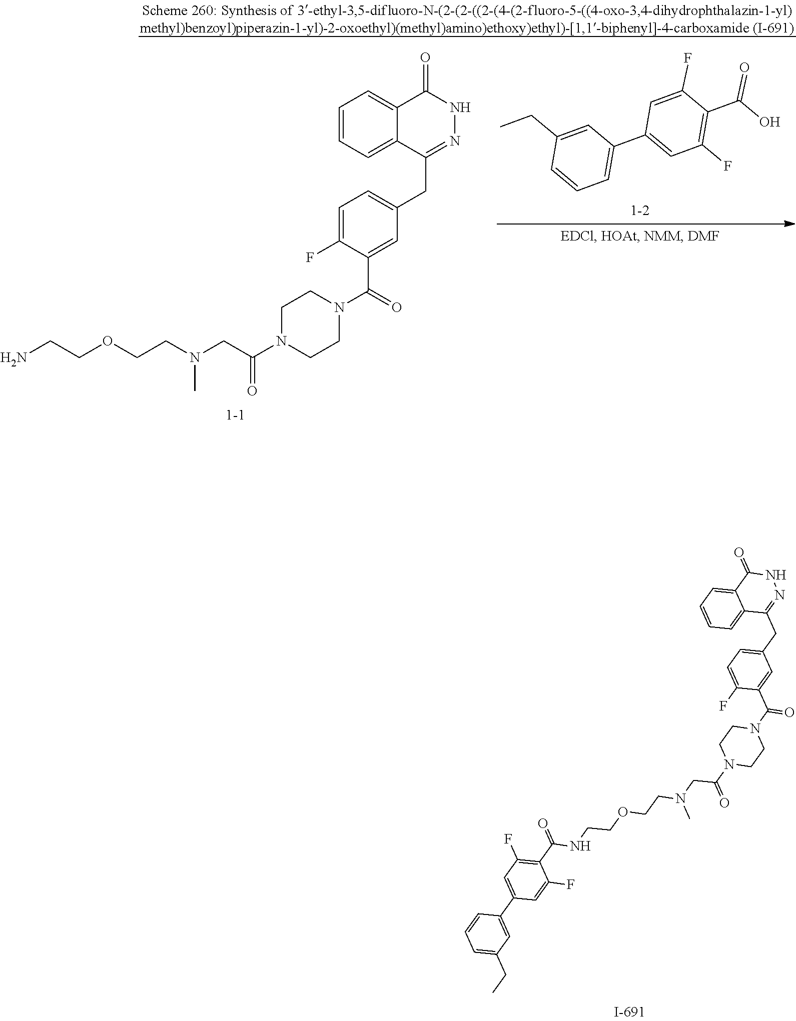 [SMILES: CN(CCOCCN)CC(=O)N1CCN(C(=O)c2cc(Cc3n[nH]c(=O)c4ccccc34)ccc2F)CC1 ; CCc1cccc(-c2cc(F)c(C(=O)O)c(F)c2)c1 ; CCc1cccc(-c2cc(F)c(C(=O)NCCOCCN(C)CC(=O)N3CCN(C(=O)c4cc(Cc5n[nH]c(=O)c6ccccc56)ccc4F)CC3)c(F)c2)c1]

To a solution of Intermediate 1-1 (1.1 g, 1.96 mmol, 1 eq, HCl salt) in DMF (11 mL) was added Intermediate 1-2 (617.01 mg, 2.35 mmol, 1.2 eq), NMM (594.93 mg, 5.88 mmol, 646.67 μL, 3 eq), EDCI (751.71 mg, 3.92 mmol, 2 eq) and HOAt (533.72 mg, 3.92 mmol, 548.53 μL, 2 eq). The mixture was stirred at 25° C. for 3 hr. The reaction mixture was combined with combined with another lot of material. The combined mixture was diluted with water (30 mL) and extracted with EA (20 mL*3). The combined organic layers were washed with sat. NaCl (20 mL), dried over Na₂SO₄, filtered and concentrated under reduced pressure to give a residue. The residue was purified by flash silica gel chromatography (ISCO; 80 g SepaFlash Silica Flash Column, Eluent of 0˜100% Ethyl acetate/Methanol @100 mL/min). The residue was purified by reversed phase HPLC (26.8*125 mm, 80 g of XB—C18, 20-40 m, 120 Å) Mobile phase: A for H₂O (0.1% NH₃·H₂O v/v) and B for acetonitrile; Gradient: B 0%-100% in 15 min; Flow rate: 40 mL/min; Column temperature: R.T. Wavelength: 220 nm/254 nm). The eluent was concentrated and lyophilized to afford the desired product. I-691 (920.03 mg, 1.19 mmol, 60.94% yield, 99.85% purity) was obtained as a white solid. LCMS (Method E): Rt=0.486 min, [M+H]⁺=769.4. ¹H NMR (400 MHz, METHANOL-d₄) δ=8.35 (d, J=7.6 Hz, 1H), 7.95-7.90 (m, 1H), 7.88-7.77 (m, 2H), 7.49-7.40 (m, 3H), 7.39 (s, 2H), 7.32-7.23 (m, 3H), 7.14-7.09 (m, 1H), 4.35 (s, 2H), 3.78 (d, J=3.2 Hz, 1H), 3.71 (s, 2H), 3.68-3.58 (m, 6H), 3.57-3.44 (m, 4H), 3.40-3.34 (m, 2H), 3.28 (d, J=2.0 Hz, 1H), 2.76-2.64 (m, 4H), 2.34 (d, J=10.0 Hz, 3H), 1.28-1.23 (m, 3H). ¹⁹F NMR (377 MHz, METHANOL-d₄) δ=−114.926, −120.683.

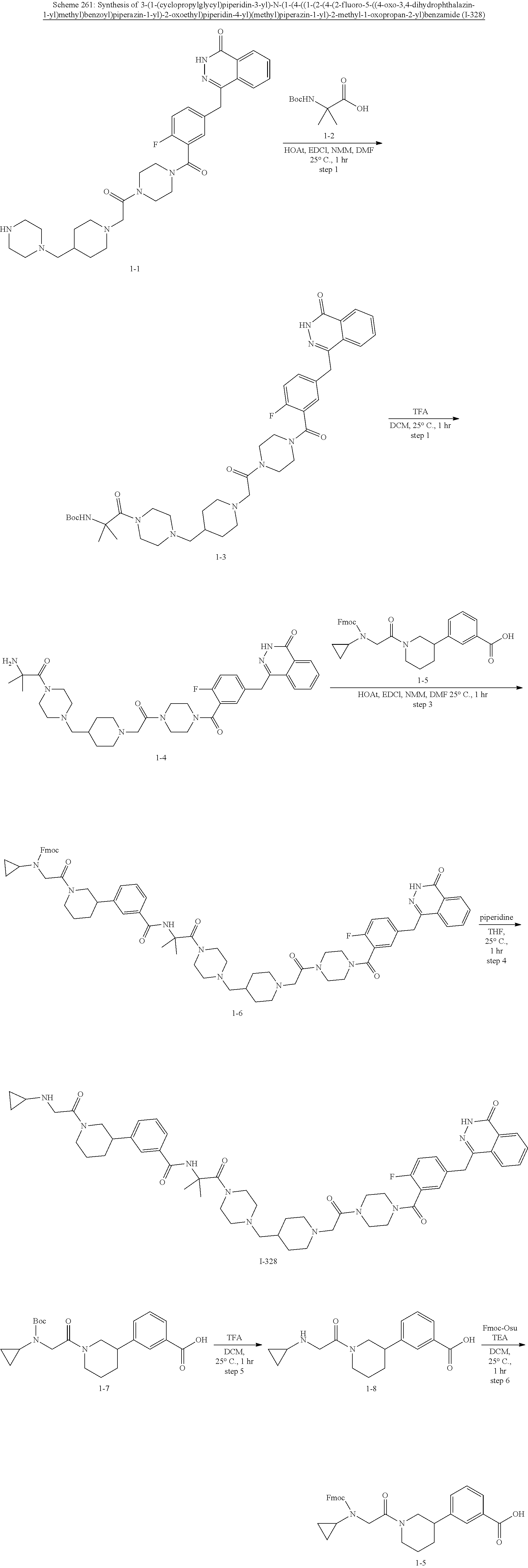

### Step 1: Synthesis of Intermediate 1-3

To a solution of Intermediate 1-2 (144.40 mg, 710.50 μmol, 1 eq) in DCM (5 mL) was added HOAt (96.71 mg, 710.50 μmol, 99.39 μL, 1 eq), EDCI (408.61 mg, 2.13 mmol, 3 eq), NMM (359.33 mg, 3.55 mmol, 390.57 μL, 5 eq) and Intermediate 1-1 (500 mg, 710.50 μmol, 1 eq, TFA salt). The mixture was stirred at 25° C. for 1 hr. The mixture was washed with water (8 mL) and extracted with DCM (5 mL*3). The combined organic layers were dried over sodium sulfate, filtered and concentrated under reduced pressure to give a residue. The residue was purified by flash silica gel chromatography (ISCO; 40 g SepaFlash Silica Flash Column, Eluent of 0˜40% EA/MeOH gradient @60 mL/min) and then the eluent was concentrated in vacuo to give Intermediate 1-3 (92 mg, 116.35 μmol, 16.38% yield, 98% purity) as white gum. LCMS (Method D): Rt=0.259 min, (M+H)⁺=775.5.

### Step 2: Synthesis of Intermediate 1-4

To a solution of Intermediate 1-3 (126 mg, 162.60 μmol, 1 eq) in DCM (1.3 mL) was added TFA (0.25 mL). The mixture was stirred at 25° C. for 1 hr. The reaction mixture was concentrated in vacuo. The crude product was used in the next step without further purification. Intermediate 1-4 (126 mg, crude, TFA salt) was obtained as yellow oil. LCMS (Method D): Rt=0.223 min, (M+H)⁺=675.4.

### Step 3: Synthesis of Intermediate 1-6

To a solution of Intermediate 1-5 (70.49 mg, 134.38 μmol, 1 eq) in DMF (1 mL) was added HOAt (18.29 mg, 134.38 μmol, 18.80 μL, 1 eq) and EDCI (77.28 mg, 403.13 μmol, 3 eq), NMM (67.96 mg, 671.88 μmol, 73.87 μL, 5 eq) and Intermediate 1-4 (106 mg, 134.38 μmol, 1 eq, TFA salt). The mixture was stirred at 25° C. for 1 hr. The mixture was washed with water (3 mL) and extracted with EA (2 mL*3). The combined organic layers were dried over sodium sulfate, filtered and concentrated under reduced pressure to give a residue. The residue was purified by prep-HPLC (FA condition), the eluent was concentrated under reduced pressure to remove ACN and lyophilized to give Intermediate 1-6 (60 mg, 45.20 μmol, 33.64% yield, 89% purity) as white solid. LCMS (Method D): Rt=0.408 min, (M+H)⁺=1181.7. ¹H NMR (400 MHz, METHANOL-d4) δ=8.39-8.33 (m, 2H), 7.97-7.91 (m, 1H), 7.90-7.77 (m, 4H), 7.75-7.63 (m, 3H), 7.55-7.44 (m, 3H), 7.43-7.30 (m, 5H), 7.19-7.12 (m, 1H), 4.83-4.73 (m, 3H), 4.58 (s, 2H), 4.55-4.45 (m, 3H), 4.38 (s, 2H), 4.31-4.12 (m, 2H), 4.04-3.89 (m, 2H), 3.82-3.62 (m, 9H), 3.57-3.47 (m, 3H), 3.42-3.34 (m, 2H), 2.73-2.59 (m, 4H), 2.46-2.29 (m, 4H), 2.21-2.13 (m, 2H), 2.09-2.03 (m, 1H), 1.91-1.80 (m, 4H), 1.58 (s, 6H), 1.45-1.24 (m, 3H), 0.74-0.48 (m, 4H),

### Step 4: Synthesis of I-328

To a solution of Intermediate 1-6 (50 mg, 42.32 μmol, 1 eq) in THF (0.5 mL) was added piperidine (63.00 L). The mixture was stirred at 25° C. for 1 hr. The reaction mixture was concentrated in vacuo. The residue was purified by prep-HPLC (basic condition), the eluent was concentrated under reduced pressure to remove ACN and lyophilized to give I-328 (19.78 mg, 20.27 μmol, 47.88% yield, 98.273% purity) as white solid. LCMS (Method D): Rt=0.253 min, (M+H)⁺=959.5. SFC: Rt=2.291 min, 3.114 min. ¹H NMR (400 MHz, METHANOL-d4) δ=8.36 (d, J=7.6 Hz, 1H), 7.99-7.91 (m, 1H), 7.90-7.80 (m, 2H), 7.75-7.67 (m, 2H), 7.52-7.42 (m, 3H), 7.37 (s, 1H), 7.16 (m, 1H), 4.62-4.57 (m, 1H), 4.38 (s, 2H), 3.95-3.87 (m, 1H), 3.79-3.63 (m, 8H), 3.59-3.49 (m, 4H), 3.27-3.08 (m, 4H), 2.90-2.70 (m, 4H), 2.35 (d, J=2.4 Hz, 3H), 2.25 (m, 1H), 2.17-2.10 (m, 2H), 2.09-2.00 (m, 3H), 1.91-1.81 (m, 2H), 1.76-1.59 (m, 4H), 1.58 (s, 6H), 1.52-1.40 (m, 1H), 1.26-1.26 (m, 1H), 1.26-1.11 (m, 2H), 0.51-0.37 (m, 4H). ¹⁹F NMR (376 MHz, METHANOL-d4) δ=−120.82.

### Step 5: Synthesis of Intermediate 1-8

A solution of Intermediate 1-7 (500 mg, 1.24 mmol, 1 eq) in TFA (1 mL) and DCM (5 mL) was stirred at 25° C. for 1 hr. The reaction mixture was concentrated in vacuo. The crude product was used in the next step without further purification. Intermediate 1-8 (500 mg, 1.15 mmol, 92.79% yield, 96% purity, TFA salt) was obtained as a white oil. LCMS (Method D): Rt=0.270 min, (M+H)⁺=303.5.

### Step 6: Synthesis of Intermediate 1-5

To a solution of Intermediate 1-8 (450 mg, 1.08 mmol, 1 eq, TFA salt) in DCM (4.5 mL) was added TEA (328.07 mg, 3.24 mmol, 451.27 μL, 3 eq) and Fmoc-OSu (437.47 mg, 1.30 mmol, 1.2 eq). The mixture was stirred at 25° C. for 1 hr. The mixture was washed with water (5 mL) and extracted with DCM (3 mL*3). The combined organic layers were dried over sodium sulfate, filtered and concentrated under reduced pressure to give a residue. The residue was purified by flash silica gel chromatography (ISCO; 40 g SepaFlash Silica Flash Column, Eluent of 0˜10% EA/MeOH gradient @60 mL/min) and then the eluent was concentrated in vacuo to give Intermediate 1-5 (175 mg, 323.58 μmol, 29.94% yield, 97% purity) as white solid. LCMS (Method D): Rt=0.476 min, (M+H)⁺=525.2.

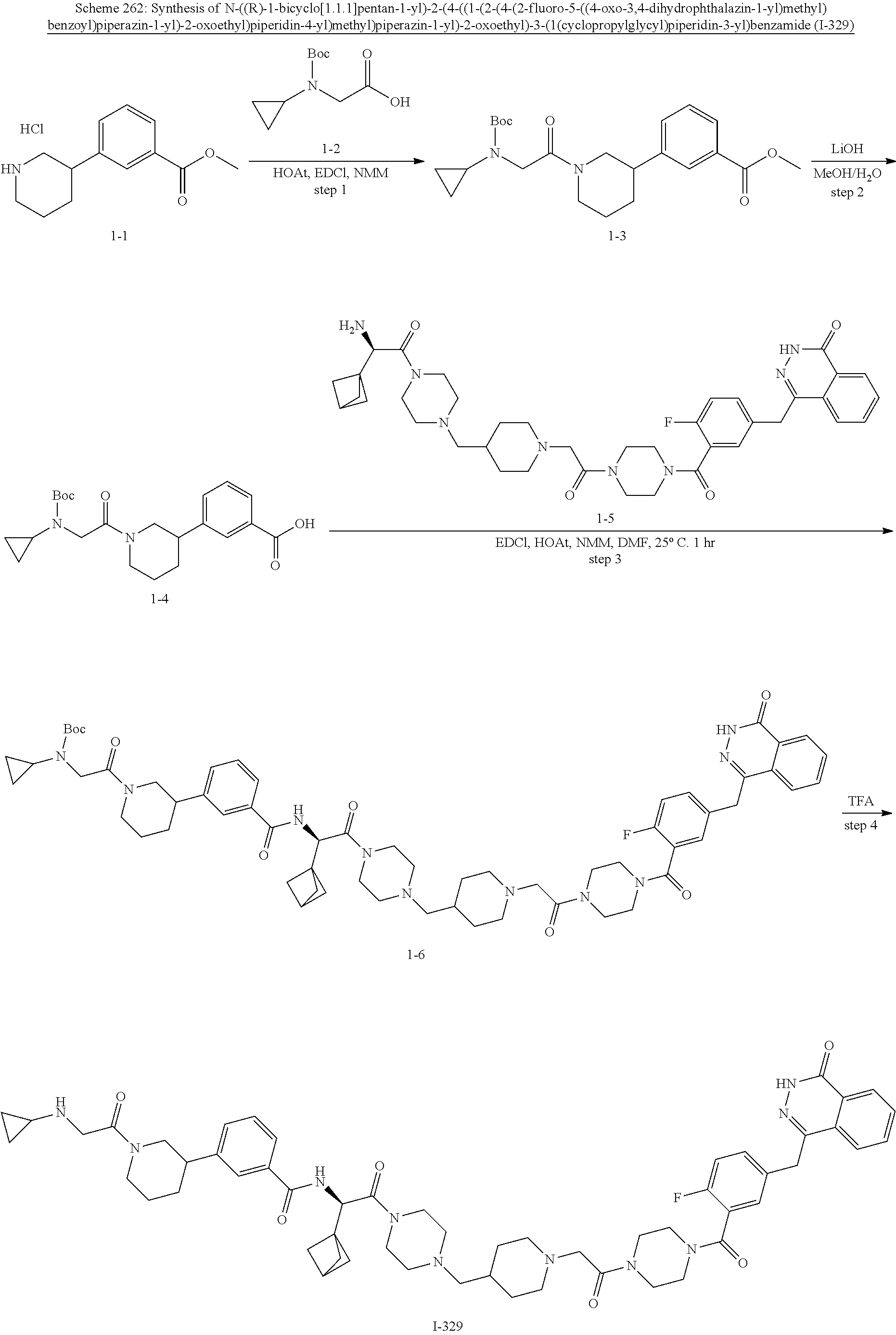

### Step 1: Synthesis of Intermediate 1-3

To a solution of Intermediate 1-1 (4 g, 15.64 mmol, 1 eq, HCl salt) and Intermediate 1-2 (3.37 g, 15.64 mmol, 1 eq) in DMF (40 mL) was added EDCI (6.00 g, 31.28 mmol, 2 eq) and HOAt (2.13 g, 15.64 mmol, 2.19 mL, 1 eq) and NMM (7.91 g, 78.20 mmol, 8.60 mL, 5 eq). The mixture was stirred at 25° C. for 1 hr. The reaction mixture was filtered and the filtrate was purified by flash silica gel chromatography. The residue was purified by flash silica gel chromatography (ISCO; 60 g SepaFlash Silica Flash Column, Eluent of 0-100% Ethyl acetate/Petroleum ethergradient @60 mL/min) and the eluent was concentrated to give Intermediate 1-3 (6.5 g, 15.61 mmol, 99.78% yield) as a white solid. LCMS (Method D): Retention time: 0.424 min, (M+Na)⁺=439.1. SFC: Retention time: 1.353, 1.439 min.

### Step 2: Synthesis of Intermediate 1-4

To a solution of Intermediate 1-3 (5.6 g, 13.45 mmol, 1 eq) in THF (22 mL) and MeOH (22 mL) and H₂O (22 mL) was added LiOH·H₂O (1.69 g, 40.34 mmol, 3 eq). The mixture was stirred at 25° C. for 1 hr. The reaction mixture was added to H₂O (60 mL) and then extracted with DCM (60 mL*3), the combined organic phase was washed, dried by Na₂SO₄, filtered and concentrated. The crude product was used in the next step. Intermediate 1-4 (5.9 g, crude) was obtained as a white solid. LCMS (Method D): Retention time: 0.469 min, (M+H)⁺=403.2.

### Step 3: Synthesis of Intermediate 1-5

To a solution of Intermediate 1-3 (38.94 mg, 96.75 μmol, 1 eq) and Intermediate 1-4 (80 mg, 96.75 μmol, 1 eq, TFA salt) in DMF (0.8 mL) was added EDCI (37.09 mg, 193.50 μmol, 2 eq) and HOAt (13.17 mg, 96.75 μmol, 13.53 μL, 1 eq) and NMM (48.93 mg, 483.75 μmol, 53.18 μL, 5 eq). The mixture was stirred at 25° C. for 1 hr. The reaction mixture was added to H₂O (2 mL) and then extracted with DCM (2 mL*3), the combined organic phase was washed, dried by Na₂SO₄, filtered and concentrated. The crude product was purified by reversed-phase HPLC (0.1% TFA condition), the eluent was concentrated to remove ACN and lyophilized to give Intermediate 1-5 (40 mg, 35.27 μmol, 36.46% yield, 96.76% purity) as a white solid. LCMS (Method D): Retention time: 1.317 min, (M+H)⁺=1097.5. SFC: Retention time: 1.399 min.

### Step 4: Synthesis of I-329

To a solution of Intermediate 1-5 (30 mg, 27.34 μmol, 1 eq) in DCM (0.3 mL) was added TFA (184.20 mg, 1.62 mmol, 120.00 μL, 59.09 eq). The mixture was stirred at 25° C. for 1 hr. The reaction mixture was concentrated under reduced pressure to give a residue, then was added to H₂O and the aqueous phase was adjusted to pH 8-9 with NaHCO₃. The crude product was purified by reversed-phase HPLC (0.1% NH₃·H₂O condition), the eluent was concentrated to remove ACN and lyophilized to give I-329 (14.38 mg, 14.22 μmol, 52.01% yield, 98.6% purity) as a white solid. LCMS (Method D): Retention time: 0.279 min, (M+H)=997.5. SFC: Retention time: 1.747, 2.277 min. ¹H NMR (400 MHz, METHANOL-d4) δ=8.37 (d, J=7.2 Hz, 1H), 7.99-7.92 (m, 1H), 7.91-7.80 (m, 2H), 7.79-7.67 (m, 2H), 7.55-7.35 (m, 4H), 7.23-7.07 (m, 1H), 5.23 (s, 1H), 4.58 (s, 2H), 4.39 (s, 2H), 3.95-3.87 (m, 1H), 3.82-3.48 (m, 13H), 3.27-3.20 (m, 2H), 2.95-2.71 (m, 4H), 2.55-2.35 (m, 5H), 2.28-2.19 (m, 3H), 2.13-2.02 (m, 3H), 1.92-1.76 (m, 10H), 1.68-1.52 (m, 2H), 1.33-1.17 (m, 3H), 0.50-0.34 (m, 4H). ¹⁹F NMR (377 MHz, METHANOL-d₄) δ=−120.8.

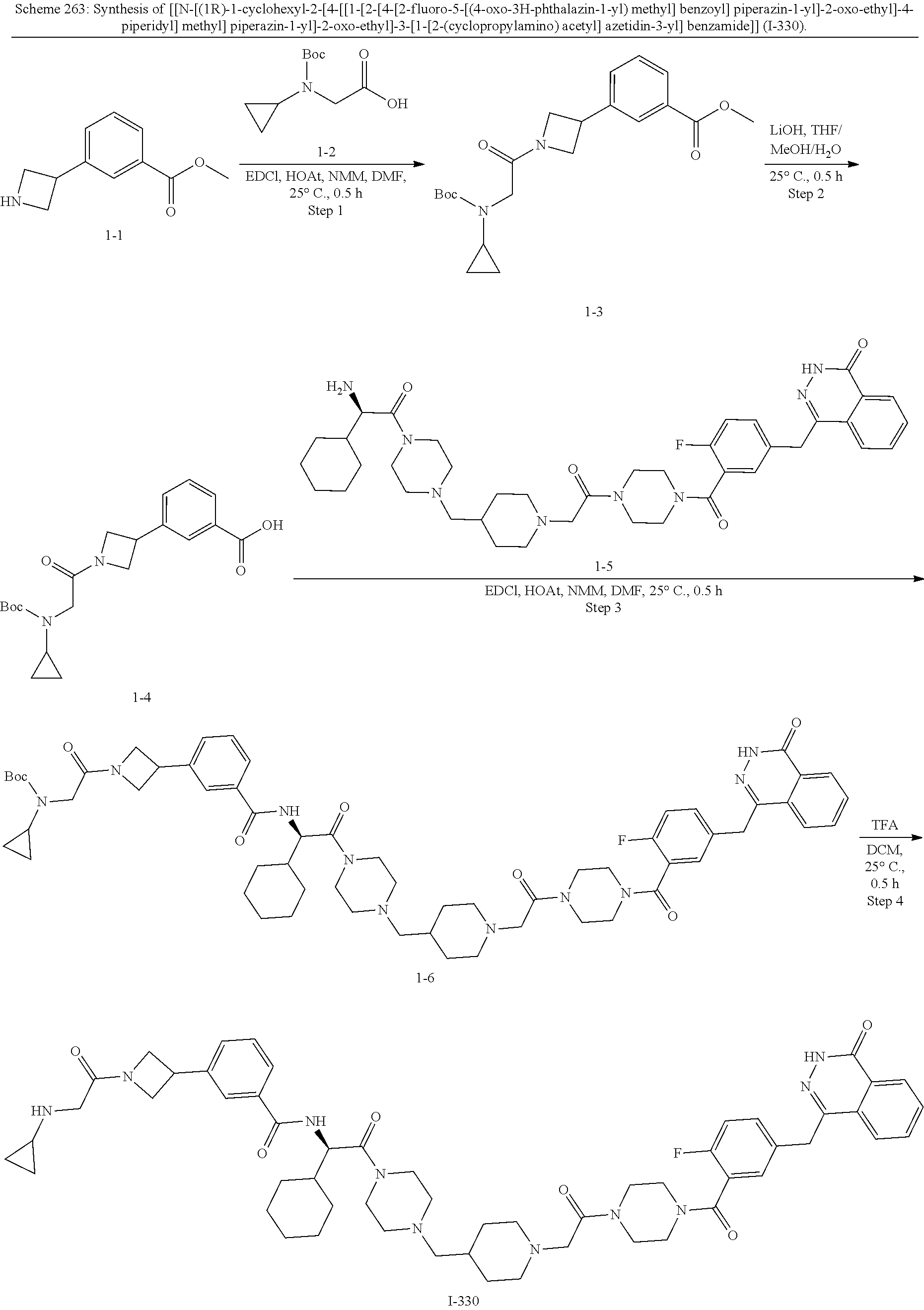

### Step 1: Synthesis of Intermediate 1-3

To a solution of intermediate 1-1 (200 mg, 1046.63 μmol, 1 eq, TFA salt) and intermediate 1-2 (169.24 mg, 786.24 μmol, 1.2 eq) in DMF (2 mL) was added EDCI (376.81 mg, 1.96 mmol, 3 eq), NMM (331.36 mg, 3.28 mmol, 360.17 μL, 5 eq) and HOAt (89.19 mg, 655.2 μmol, 91.65 μL, 1 eq). The mixture was stirred at 25° C. for 0.5 hr. The reaction mixture was poured into H₂O (10 mL), and extracted with EA (10 mL*3). The combined organic layers were dried over Na₂SO₄, filtered and concentrated under reduced pressure to give a residue. The residue was purified by flash silica gel chromatography (ISCO; 12 g SepaFlash Silica Flash Column, Eluent of 0˜100% PE/EA@36 mL/min, PE/EA=3:1, Rf=0.2) and the eluent was concentrated under reduced pressure to give Intermediate 1-3 (300 mg, crude) as a liquid. LCMS (Method D): Rt=0.438 min, (M+H)=289.1.

### Step 2: Synthesis of Intermediate 1-4

To a solution of intermediate 1-3 (382 mg, 983.38 μmol, 1 eq) in MeOH (1.5 mL), THF (1.5 mL) and H₂O (1.5 mL) was added LiOH·H₂O (123.80 mg, 2.95 mmol, 3 eq). The mixture was stirred at 25° C. for 0.5 hr. The reaction mixture was poured into H₂O (10 mL) at 25° C., the pH was adjusted to 3 with 1M aqueous HCl, and then extracted with DCM (10 mL*3). The combined organic layers were dried over Na₂SO₄, filtered and concentrated under reduced pressure to give Intermediate 1-4 (280 mg, crude) as a yellow gum. LCMS (Method D): Rt=0.394 min, (M+H)⁺=275.2.

### Step 3: Synthesis of Intermediate 1-6

To a solution of intermediate 1-5 (50 mg, 65.33 μmol, 1 eq, HCl salt) and intermediate 1-4 (26.91 mg, 71.86 μmol, 1.1 eq) in DMF (0.5 mL) was added EDCI (37.57 mg, 195.99 μmol, 3 eq), NMM (33.04 mg, 326.64 μmol, 35.91 μL, 5 eq) and HOAt (8.89 mg, 65.33 μmol, 9.14 μL, 1 eq). The mixture was stirred at 25° C. for 0.5 hr. The reaction mixture was poured into H₂O (2 mL), and extracted with EA (2 mL*3). The combined organic layers were dried over Na₂SO₄, filtered and concentrated under reduced pressure to give a residue. The residue was purified by flash silica gel chromatography (ISCO; 4 g SepaFlashSilica Flash Column, Eluent of 0˜100% DCM/MeOH@18 mL/min, DCM/MeOH=10:1, Rf=0.2) and the eluent was concentrated under reduced pressure to give Intermediate 1-6 (50 mg, crude) as a yellow gum. LCMS (Method D): Rt=0.361 min, (M+H)⁺=1085.8.

### Step 4: Synthesis of I-330

To a solution of intermediate 1-6 (50 mg, 46.07 μmol, 1 eq) in DCM (0.5 mL) was added TFA (153.50 mg, 1.35 mmol, 0.1 mL, 29.22 eq). The mixture was stirred at 25° C. for 0.5 hr. The reaction mixture was concentrated under reduced pressure to remove solvent. The residue was purified by prep-HPLC (column: Phenomenex Luna C18 150*25 mm*10 um; mobile phase: [water (FA)-ACN]; gradient: 12%-42% B over 10 min) and concentrated under reduced pressure to remove ACN, lyophilized to give I-330 (18.32 mg, 16.57 μmol, 35.96% yield, 89.0% purity) as a white solid. LCMS (Method D): Rt=0.267 min, (M+H)⁺=985.5. ¹H NMR (400 MHz, METHANOL-d4) δ=8.37 (d, J=7.2 Hz, 1H), 7.99-7.92 (m, 1H), 7.91-7.81 (m, 3H), 7.77 (d, J=7.6 Hz, 1H), 7.63-7.57 (m, 1H), 7.52 (d, J=7.6 Hz, 2H), 7.42-7.32 (m, 1H), 7.18 (t, J=8.8 Hz, 1H), 4.68 (t, J=8.8 Hz, 1H), 4.55-4.49 (m, 1H), 4.39 (s, 2H), 4.35-4.21 (m, 4H), 4.19-4.06 (m, 3H), 4.01 (s, 3H), 3.87-3.62 (m, 6H), 3.60-3.44 (m, 4H), 3.43-3.33 (m, 5H), 3.23-3.02 (m, 5H), 2.85-2.77 (m, 1H), 2.26-2.04 (m, 3H), 1.99-1.89 (m, 2H), 1.84-1.65 (m, 6H), 1.36-1.22 (m, 3H), 1.19-1.07 (m, 2H), 0.92 (d, J=5.2 Hz, 4H). F NMR (376 MHz, METHANOL-d4) δ=−120.755.

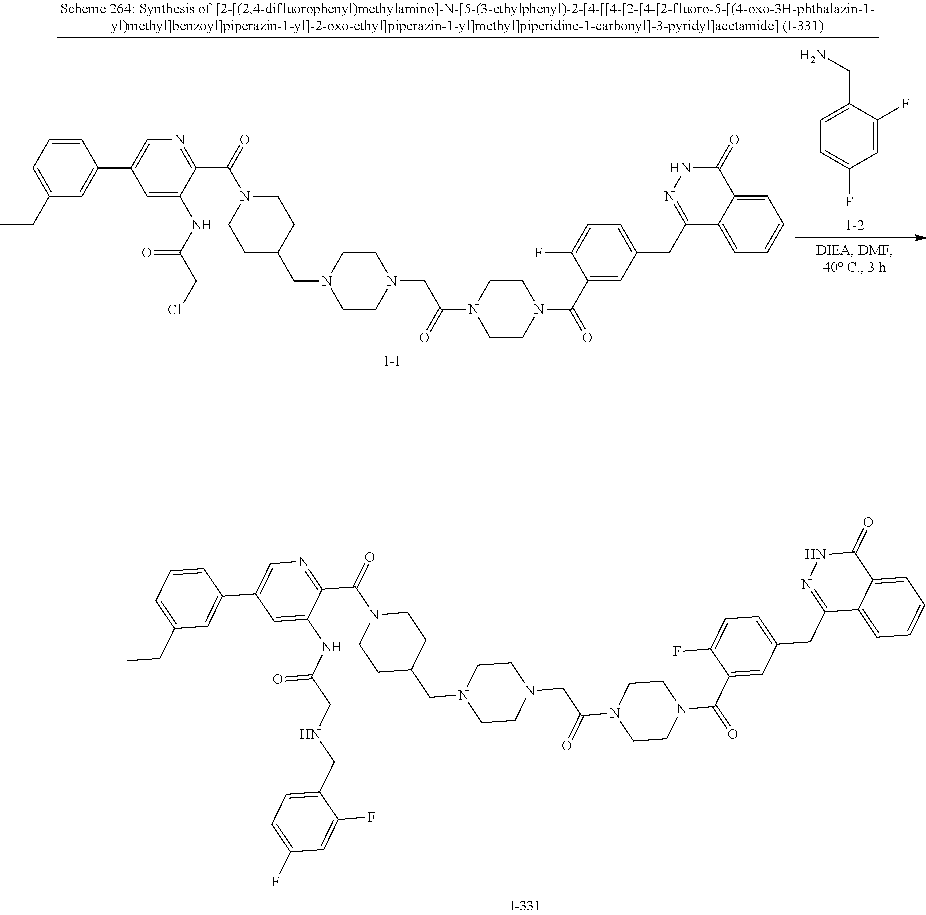 [SMILES: CCc1cccc(-c2cnc(C(=O)N3CCC(CN4CCN(CC(=O)N5CCN(C(=O)c6cc(Cc7n[nH]c(=O)c8ccccc78)ccc6F)CC5)CC4)CC3)c(NC(=O)CCl)c2)c1 ; NCc1ccc(F)cc1F ; CCc1cccc(-c2cnc(C(=O)N3CCC(CN4CCN(CC(=O)N5CCN(C(=O)c6cc(Cc7n[nH]c(=O)c8ccccc78)ccc6F)CC5)CC4)CC3)c(NC(=O)CNCc3ccc(F)cc3F)c2)c1]

To a mixture of intermediate 1-1 (150 mg, 168.46 μmol, 1 eq) in DMF (1.5 mL) was added intermediate 1-2 (28.93 mg, 202.15 μmol, 1.2 eq) and DIEA (43.54 mg, 336.91 μmol, 58.68 μL, 2 eq) at 25° C. and the mixture was stirred at 40° C. for 1 hr. The reaction mixture was concentrated to give a residue. The residue was purified by prep-HPLC (column: Phenomenex luna C18 150*25 mm*10 um; mobile phase: [water (FA)-ACN]; gradient: 13%-43% B over 10 min) and lyophilized to give I-331 (19.93 mg, 18.60 μmol, 11.04% yield, 97.3% purity, FA salt) as a purple solid. LCMS (Method D): Retention time: 0.329 min, (M+1)⁺=997.3. ¹H NMR (400 MHz, METHANOL-d4) δ=8.77 (d, J=1.6 Hz, 1H), 8.60 (d, J=1.6 Hz, 1H), 8.52 (s, 1H), 8.41-8.34 (m, 1H), 7.97-7.92 (m, 1H), 7.91-7.80 (m, 2H), 7.65-7.55 (m, 1H), 7.54-7.46 (m, 3H), 7.43 (t, J=7.6 Hz, 1H), 7.37 (t, J=7.2 Hz, 1H), 7.31 (d, J=7.6 Hz, 1H), 7.20-7.12 (m, 1H), 7.01-6.90 (m, 2H), 4.68-4.56 (m, 2H), 4.38 (s, 2H), 3.87 (s, 2H), 3.82-3.63 (m, 6H), 3.57-3.48 (m, 2H), 3.43 (s, 2H), 3.25-3.09 (m, 3H), 2.87 (t, J=12.8 Hz, 1H), 2.78-2.70 (m, 2H), 2.54 (s, 8H), 2.30-2.20 (m, 2H), 1.88 (d, J=4.0 Hz, 2H), 1.80-1.69 (m, 1H), 1.32-1.18 (m, 5H).

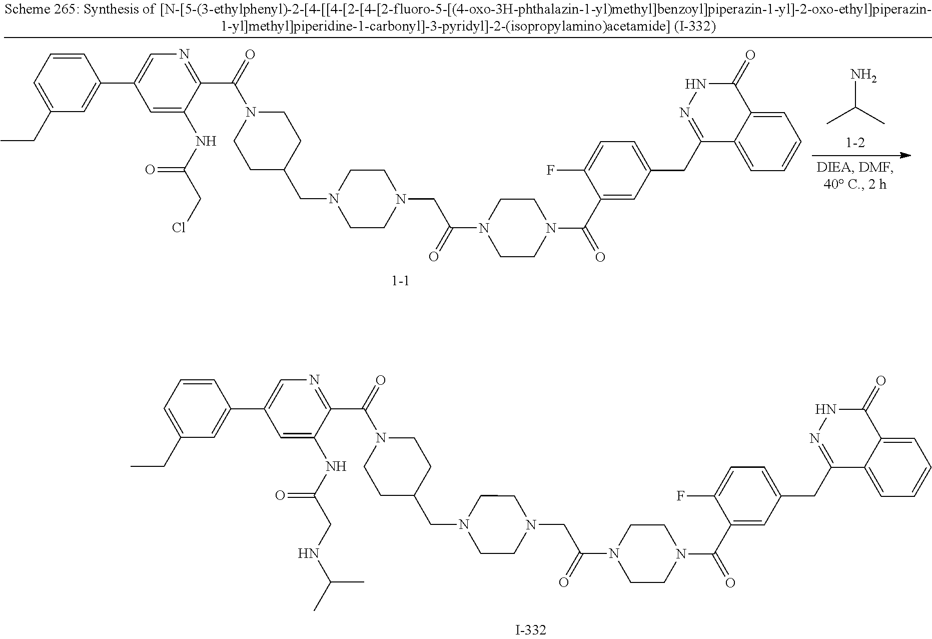

To a mixture of intermediate 1-1 (70 mg, 78.61 μmol, 1 eq) in DMF (1 mL) was added intermediate 1-2 (5.58 mg, 94.34 μmol, 8.10 μL, 1.2 eq) and DIEA (20.32 mg, 157.23 μmol, 27.39 μL, 2 eq) at 25° C. and the mixture was stirred at 40° C. for 1 h. The reaction mixture was concentrated to give a residue. The residue was purified by prep-HPLC (column: Phenomenex luna C18 150*25 mm*10 um; mobile phase: [water (FA)-ACN]; gradient: 7%-37% B over 8 min) and lyophilized to give I-332 (20 mg, 21.90 μmol, 27.86% yield) as a white solid. LCMS (Method D): Retention time: 0.335 min, (M+H)⁺=913.5. ¹H NMR (400 MHz, METHANOL-d4) δ=8.70 (s, 1H), 8.61 (s, 1H), 8.50 (s, 1H), 8.37 (d, J=7.2 Hz, 1H), 7.94 (d, J=5.2 Hz, 1H), 7.91-7.79 (m, 2H), 7.60-7.46 (m, 3H), 7.43 (t, J=7.6 Hz, 1H), 7.40-7.34 (m, 1H), 7.31 (d, J=7.6 Hz, 1H), 7.16 (t, J=8.8 Hz, 1H), 4.68 (d, J=14.0 Hz, 2H), 4.38 (s, 2H), 3.82-3.65 (m, 5H), 3.61-3.48 (m, 4H), 3.25 (s, 3H), 3.15 (t, J=12.4 Hz, 1H), 3.06-2.97 (m, 1H), 2.92 (t, J=12.8 Hz, 1H), 2.78-2.71 (m, J=7.6 Hz, 2H), 2.58 (d, J=2.4 Hz, 8H), 2.31 (d, J=8.8 Hz, 2H), 1.93 (s, 2H), 1.76 (d, J=12.0 Hz, 1H), 1.29 (t, J=7.6 Hz, 5H), 1.19 (d, J=6.0 Hz, 6H). ¹⁹F NMR (377 MHz, METHANOL-d₄) δ=−120.8.

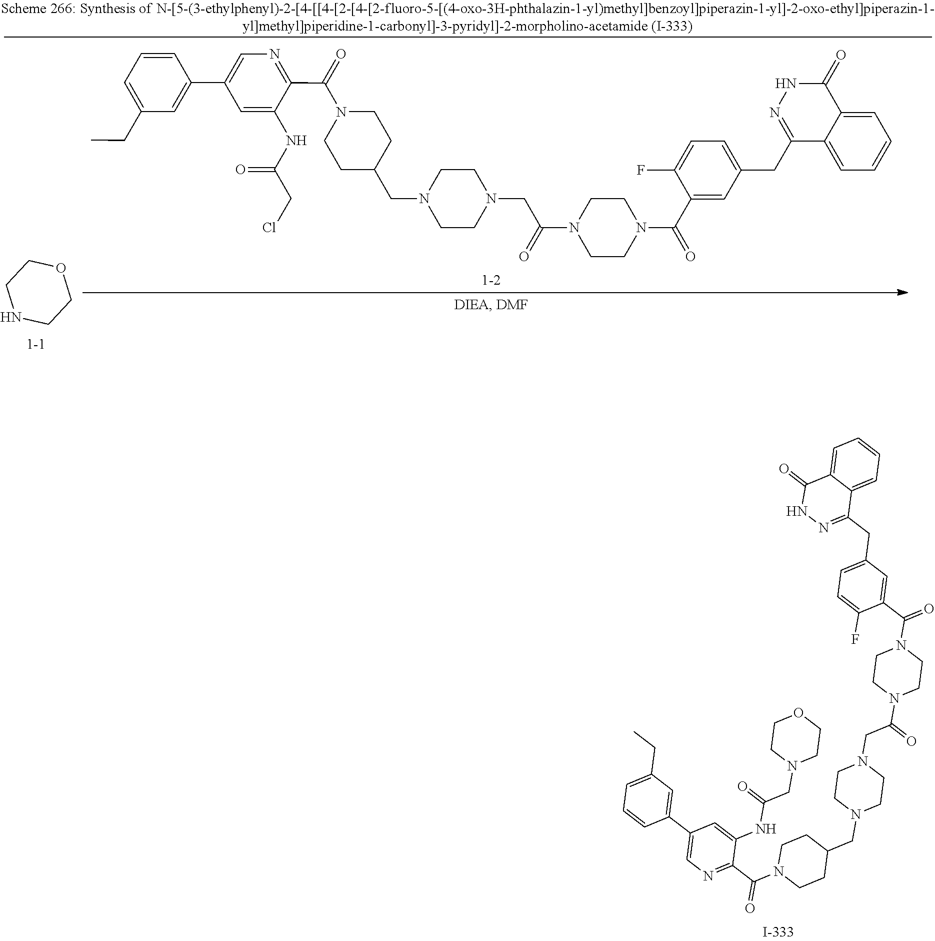 [SMILES: CCc1cccc(-c2cnc(C(=O)N3CCC(CN4CCN(CC(=O)N5CCN(C(=O)c6cc(Cc7n[nH]c(=O)c8ccccc78)ccc6F)CC5)CC4)CC3)c(NC(=O)CCl)c2)c1 ; C1COCCN1 ; CCc1cccc(-c2cnc(C(=O)N3CCC(CN4CCN(CC(=O)N5CCN(C(=O)c6cc(Cc7n[nH]c(=O)c8ccccc78)ccc6F)CC5)CC4)CC3)c(NC(=O)CN3CCOCC3)c2)c1]

To a solution of Intermediate 1-1 (6.85 mg, 78.61 μmol, 6.92 μL, 1 eq) and Intermediate 1-2 (70 mg, 78.61 μmol, 1 eq) in DMF (1 mL) was added DIEA (20.32 mg, 157.23 μmol, 27.39 μL, 2 eq). The mixture was stirred at 40° C. for 1 hr. The reaction mixture was diluted with water (5 mL) and extracted with EA (5 mL*3). The combined organic layers were dried over Na₂SO₄, filtered and concentrated under reduced pressure to give a residue. The residue was purified by prep-HPLC (column: CD02-Waters Xbidge BEH C18 150*25*10 um; mobile phase: [water (NH₃H₂O)-ACN]; gradient: 34%-64% B over 11 min). The eluent was lyophilized to give I-333 (15 mg, 15.94 μmol, 20.28% yield) as a white solid. LCMS (Method D): Rt=0.333 min, [M+H]⁺=941.8. ¹H NMR (400 MHz, METHANOL-d₄) δ=8.88 (s, 1H), 8.60 (s, 1H), 8.37 (d, J=7.6 Hz, 1H), 7.99-7.92 (m, 1H), 7.91-7.80 (m, 2H), 7.55-7.47 (m, 3H), 7.45-7.35 (m, 2H), 7.31 (d, J=7.2 Hz, 1H), 7.16 (t, J=8.8 Hz, 1H), 4.78-4.66 (m, 2H), 4.39 (s, 2H), 3.88-3.62 (m, 10H), 3.58-3.48 (m, 2H), 3.23-3.10 (m, 4H), 3.00-2.90 (m, 1H), 2.78-2.72 (m, 2H), 2.63 (s, 4H), 2.59-2.43 (m, 7H), 2.29-2.20 (m, 2H), 2.01-1.87 (m, 2H), 1.82-1.72 (m, 1H), 1.34-1.21 (m, 7H). ¹⁹F NMR: (377 MHz, METHANOL-d₄) δ=−120.8.

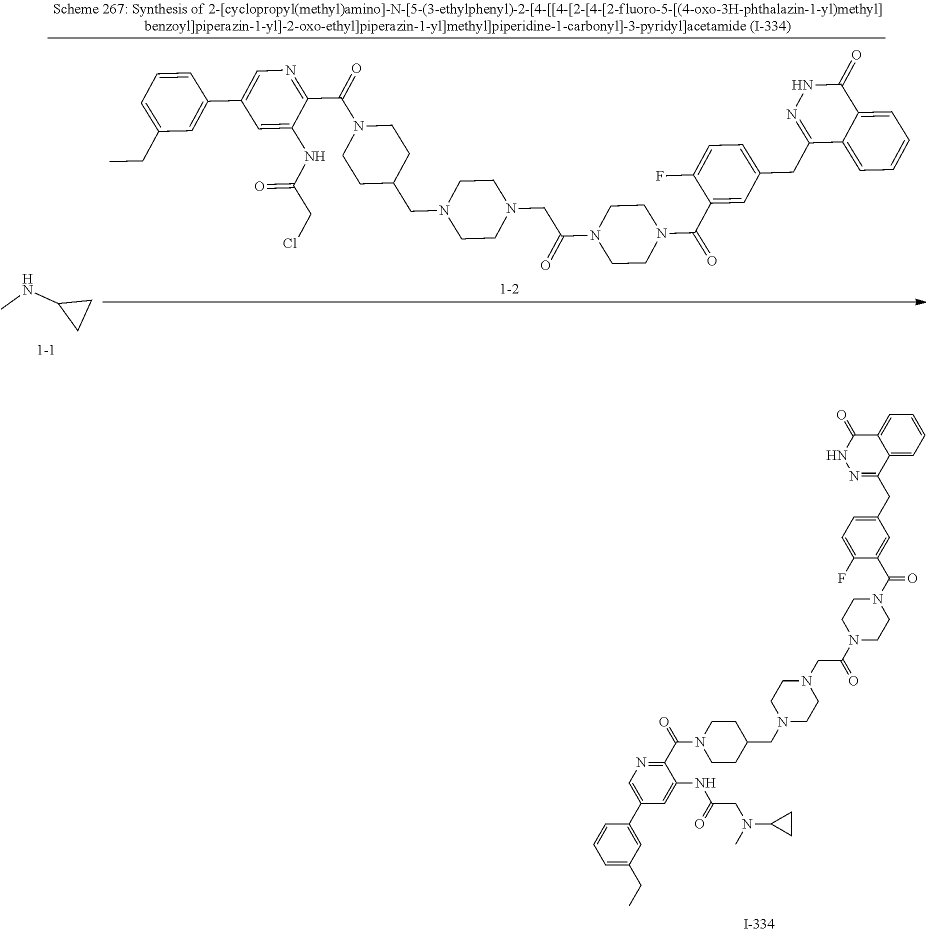

To a solution of Intermediate 1-1 (5.59 mg, 78.61 μmol, 1 eq) and Intermediate 1-2 (70 mg, 78.61 μmol, 1 eq) in DMF (1 mL) was added DIEA (20.32 mg, 157.23 μmol, 27.39 μL, 2 eq). The mixture was stirred at 40° C. for 1.5 hr. The reaction mixture was diluted with water (5 mL) and extracted with EA (5 mL*3). The combined organic layers were dried over Na₂SO₄, filtered and concentrated under reduced pressure to give a residue. The residue was purified by prep-HPLC (column: CD02-Waters Xbidge BEH C18 150*25*10 um; mobile phase: [water(NH₃H₂O)-ACN]; gradient: 43%-73% B over 11 min). The eluent was lyophilized to give (I-334) (15 mg, 16.21 μmol, 20.63% yield, 100% purity) as a white solid. LCMS (Method D): Rt=0.368 min, [M+H]⁺=925.4. ¹H NMR (400 MHz, METHANOL-d4) δ=8.86 (s, 1H), 8.58 (s, 1H), 8.37 (d, J=7.6 Hz, 1H), 7.99-7.92 (m, 1H), 7.92-7.80 (m, 2H), 7.56-7.46 (m, 3H), 7.45-7.34 (m, 2H), 7.31 (d, J=7.2 Hz, 1H), 7.16 (t, J=9.2 Hz, 1H), 4.74-4.66 (m, 2H), 4.39 (s, 2H), 3.82-3.71 (m, 4H), 3.67 (d, J=1.6 Hz, 1H), 3.58-3.49 (m, 2H), 3.37 (s, 2H), 3.27 (s, 1H), 3.22-3.12 (m, 2H), 2.97-2.89 (m, 1H), 2.77-2.72 (m, 2H), 2.57-2.45 (m, 9H), 2.28-2.19 (m, 2H), 2.09-2.05 (m, 1H), 2.00-1.88 (m, 2H), 1.81-1.73 (m, 1H), 1.34-1.22 (m, 9H), 0.57 (d, J=4.4 Hz, 3H). ¹⁹F NMR (376 MHz, METHANOL-d₄) δ=−120.8.

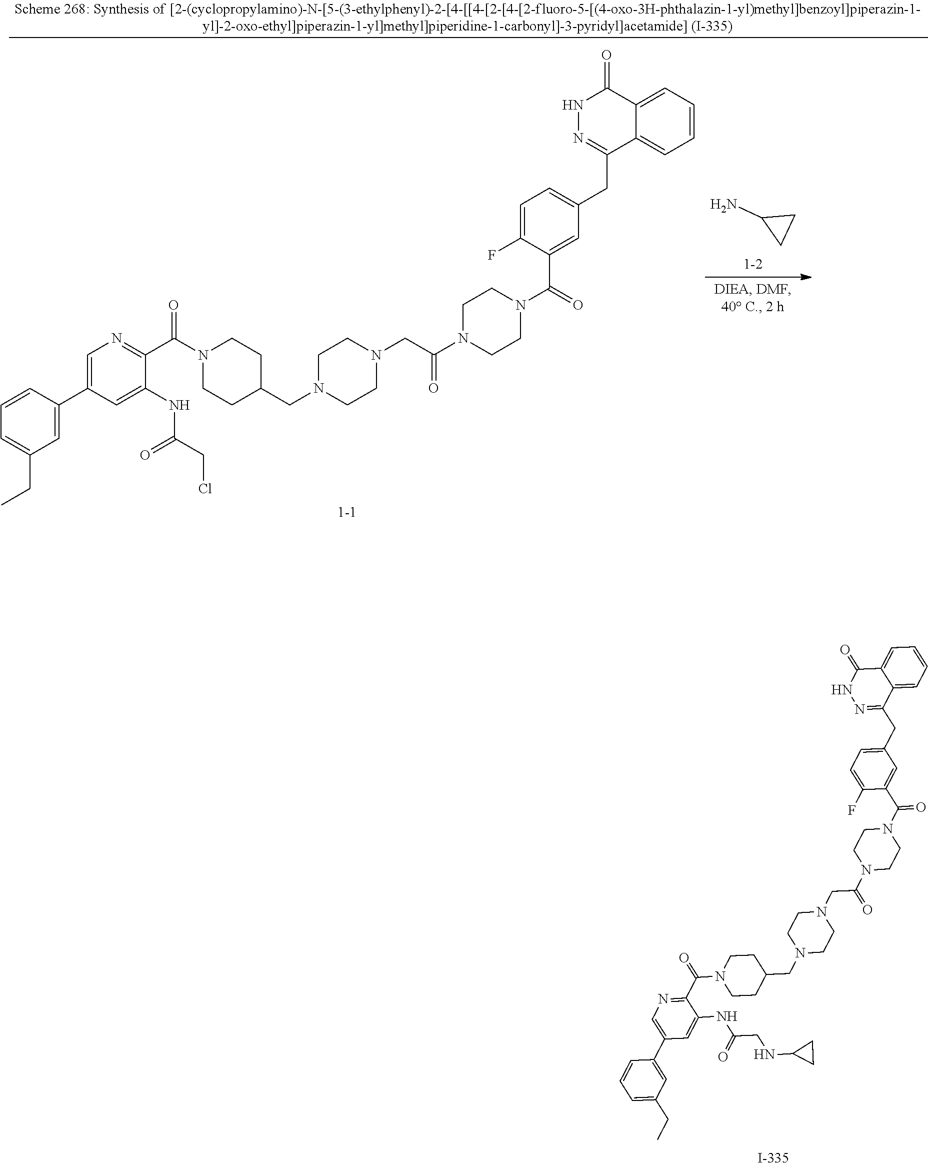

To a solution of intermediate 1-1 (50 mg, 56.15 μmol, 1 eq) and intermediate 1-2 (4.81 mg, 84.23 μmol, 5.84 μL, 1.5 eq) in DMF (0.5 mL) was added DIEA (14.51 mg, 112.30 μmol, 19.56 μL, 2 eq). The mixture was stirred at 40° C. for 2 hr. The mixture was poured into 5 mL of H₂O and then extracted with EA (5 mL*3). The combined organic layers were dried over Na₂SO₄, filtered and concentrated under reduced pressure to give a residue. The residue was purified by reversed-phase HPLC (0.1% FA condition). The eluent was concentrated under reduced pressure to remove ACN, then lyophilized to afford I-335 (16 mg, 16.50 μmol, 29.38% yield, 93.93% purity) as a white solid. LCMS (Method D): Rt=0.318 min, [M+H]⁺=911.4.

¹H NMR (400 MHz, METHANOL-d4) δ=8.75 (d, J=2.0 Hz, 1H), 8.59 (d, J=2.0 Hz, 1H), 8.42-8.33 (m, 2H), 7.99-7.92 (m, 1H), 7.91-7.80 (m, 2H), 7.54-7.48 (m, 2H), 7.43 (t, J=7.6 Hz, 1H), 7.38-7.34 (m, 1H), 7.31 (br d, J=7.6 Hz, 1H), 7.17 (t, J=9.2 Hz, 1H), 4.70 (br d, J=12.4 Hz, 2H), 4.39 (s, 2H), 3.84-3.63 (m, 5H), 3.54-3.46 (m, 4H), 3.41 (br s, 1H), 3.26-3.20 (m, 1H), 3.18-3.11 (m, 1H), 2.99-2.87 (m, 2H), 2.85-2.68 (m, 8H), 2.62-2.48 (m, 2H), 2.40-2.29 (m, 1H), 2.07-1.91 (m, 2H), 1.77 (br d, J=11.6 Hz, 1H), 1.41-1.34 (m, 5H), 1.29 (t, J=7.6 Hz, 3H), 0.55-0.45 (m, 3H). ¹⁹F NMR: (377 MHz, METHANOL-d₄) δ=−120.785.

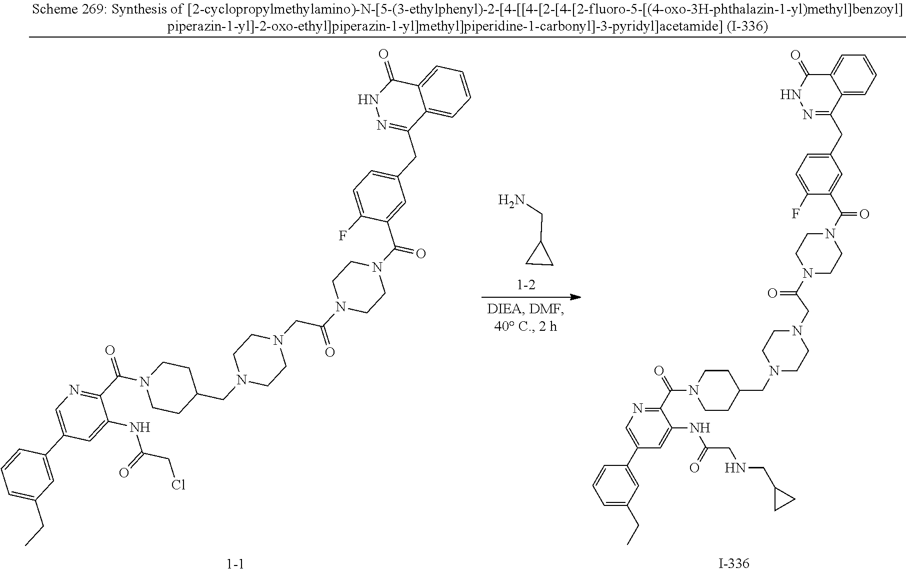 [SMILES: CCc1cccc(-c2cnc(C(=O)N3CCC(CN4CCN(CC(=O)N5CCN(C(=O)c6cc(Cc7n[nH]c(=O)c8ccccc78)ccc6F)CC5)CC4)CC3)c(NC(=O)CCl)c2)c1 ; NCC1CC1 ; CCc1cccc(-c2cnc(C(=O)N3CCC(CN4CCN(CC(=O)N5CCN(C(=O)c6cc(Cc7n[nH]c(=O)c8ccccc78)ccc6F)CC5)CC4)CC3)c(NC(=O)CNCC3CC3)c2)c1]

To a solution of intermediate 1-1 (50 mg, 56.15 μmol, 1 eq) and intermediate 1-2 (5.99 mg, 84.23 μmol, 1.5 eq) in DMF (0.5 mL) was added DIEA (14.51 mg, 112.30 μmol, 19.56 μL, 2 eq). The mixture was stirred at 40° C. for 2 hr. The mixture was poured into 5 mL of H₂O and then extracted with EA (5 mL*3). The combined organic layers were dried over Na₂SO₄, filtered and concentrated under reduced pressure to give a residue. The crude product was purified by reversed-phase HPLC (0.1% TFA condition). The eluent was concentrated under reduced pressure to remove ACN, then lyophilized to afford I-336 (10 mg, 8.94 μmol, 15.93% yield, 92.946% purity, TFA salt) as a white solid. LCMS (Method D): Rt=0.327 min, [M+H]⁺=925.4. ¹H NMR (400 MHz, METHANOL-d4) δ=8.65 (s, 1H), 8.50 (br s, 2H), 8.37 (br d, J=7.6 Hz, 1H), 7.99-7.92 (m, 1H), 7.91-7.80 (m, 2H), 7.54-7.48 (m, 2H), 7.44 (br t, J=7.6 Hz, 1H), 7.40-7.29 (m, 2H), 7.17 (brt, J=9.2 Hz, 1H), 4.69-4.59 (m, 3H), 4.39 (s, 2H), 3.82 (br s, 3H), 3.76-3.66 (m, 3H), 3.52 (br s, 2H), 3.38 (br s, 1H), 3.25-3.11 (m, 3H), 2.97-2.86 (m, 1H), 2.82 (br d, J=7.2 Hz, 2H), 2.79-2.71 (m, 3H), 2.66 (br d, J=15.2 Hz, 6H), 2.44-2.39 (m, 2H), 1.95 (br d, J=5.6 Hz, 2H), 1.77-1.73 (m, 1H), 1.43-1.32 (m, 3H), 1.29 (t, J=7.6 Hz, 3H), 1.17-1.02 (m, 1H), 0.66 (br d, J=7.6 Hz, 2H), 0.35 (br d, J=5.2 Hz, 2H). ¹⁹F NMR: (377 MHz, METHANOL-d₄) δ=−120.710.

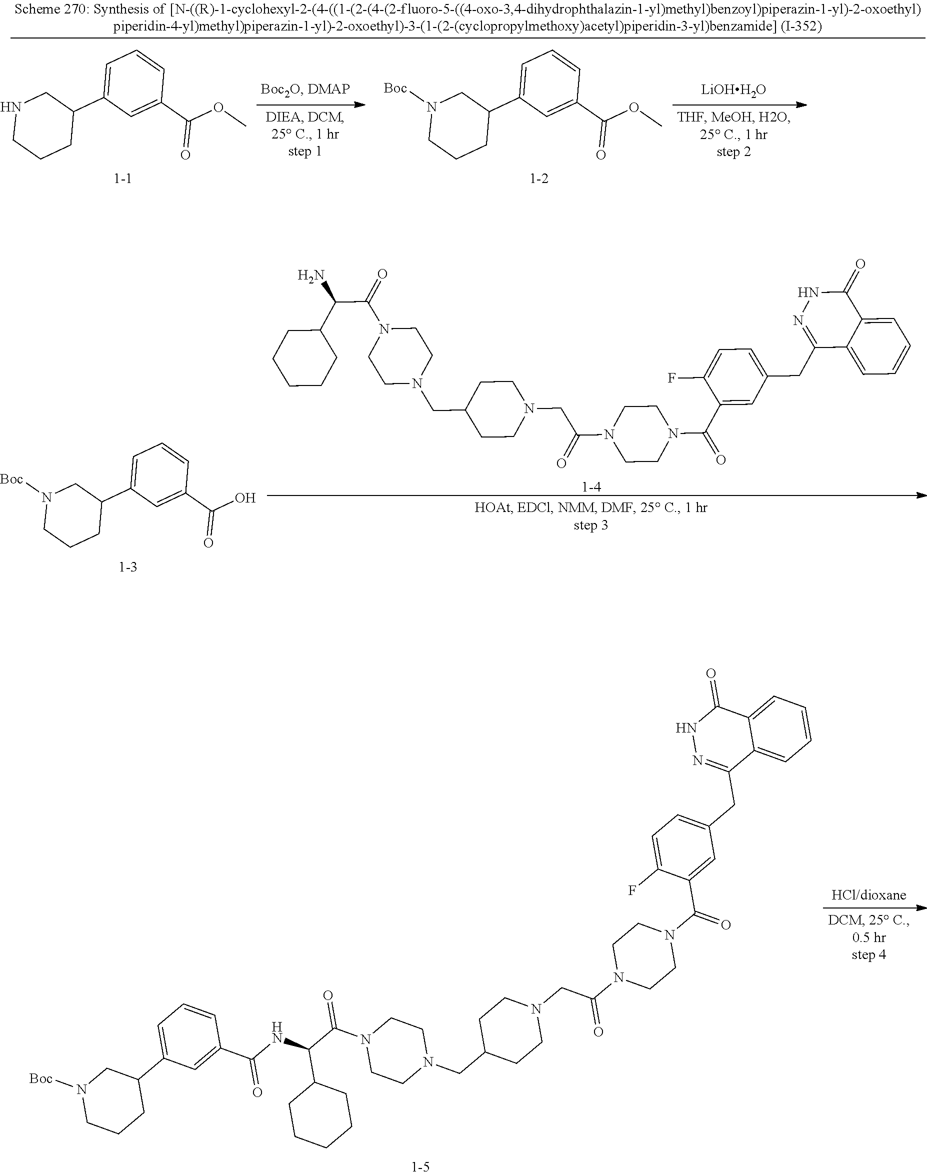

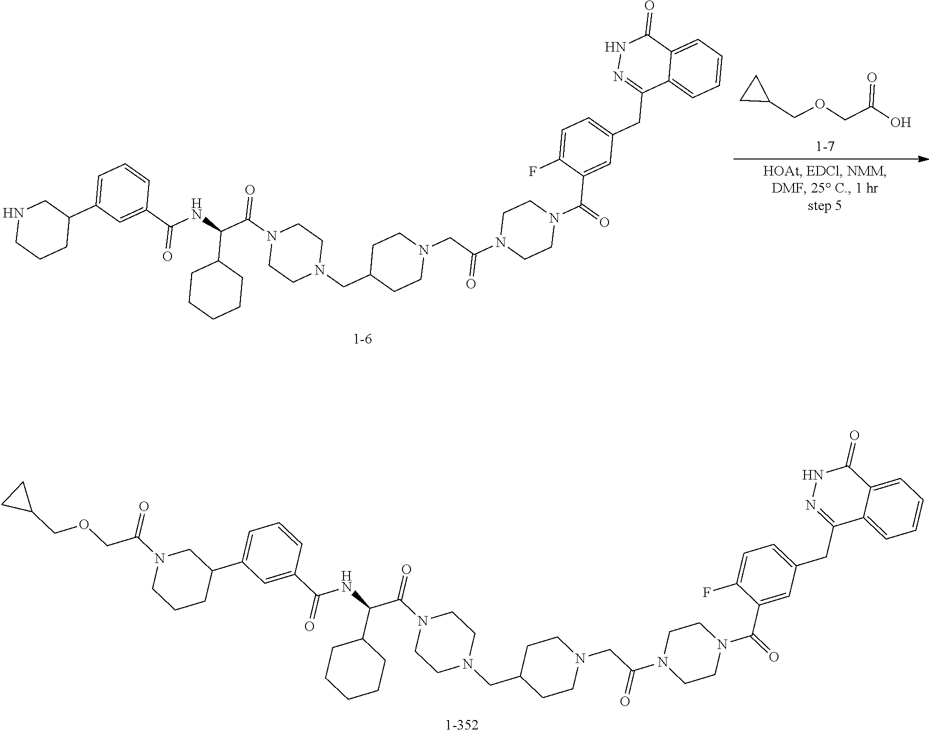

### Step 1: Synthesis of Intermediate 1-2.

To a solution of Intermediate 1-1 (5 g, 22.80 mmol, 1 eq) in DCM (50 mL) was added DIEA (8.84 g, 68.41 mmol, 11.92 mL, 3 eq), Boc₂O (5.97 g, 27.36 mmol, 6.29 mL, 1.2 eq) and DMAP (278.57 mg, 2.28 mmol, 0.1 eq). The mixture was stirred at 25° C. for 1 hr. The reaction mixture was diluted with H₂O (20 mL) and extracted with EA (10 mL*3). The combined organic layers were washed with brine (10 mL*3), dried over Na₂SO₄, filtered and concentrated under reduced pressure to give a residue. The residue was purified by flash silica gel chromatography (ISCO; 120 g SepaFlash Silica Flash Column, Eluent of 0˜20% Ethyl acetate/Petroleum ethergradient @100 mL/min), the eluent was concentrated to give Intermediate 1-2 (7 g, 21.92 mmol, 96.12% yield, 100% purity) as a yellow oil. LCMS (Method D): Retention time: 0.443 min, (M+Na)⁺=342.1. ¹H NMR (400 MHz, CHLOROFORM-d) δ=7.95-7.86 (m, 2H), 7.47-7.36 (m, 2H), 4.26-4.09 (m, 2H), 3.92 (s, 3H), 2.75 (d, J=9.2 Hz, 2H), 1.78 (d, J=12.4 Hz, 1H), 1.72-1.60 (m, 2H), 1.47 (s, 9H), 1.11-0.79 (m, 2H).

### Step 2: Synthesis of Intermediate 1-3.

To a solution of Intermediate 1-2 (7 g, 21.92 mmol, 1 eq) in THF (20 mL), MeOH (20 mL) and H₂O (20 mL) was added LiOH·H₂O (2.76 g, 65.75 mmol, 3 eq). The mixture was stirred at 25° C. for 1 hr. The reaction mixture was adjusted pH to 6-7 with 1 N HCl, and then was diluted with H₂O (5 mL) and extracted with EA (5 mL*3). The combined organic layers were washed with brine (5 mL*3), dried over Na₂SO₄, filtered and concentrated under reduced pressure to give a residue. The crude product was used in the next step without further purification. Intermediate 1-3 (6 g, 19.38 mmol, 88.41% yield, 98.6% purity) was obtained as a yellow oil LCMS (Method F): Retention time: 0.494 min, (M+Na)⁺=328.1. ¹H NMR (400 MHz, CHLOROFORM-d) δ=7.82 (s, 1H), 7.75 (s, 1H), 7.17 (s, 2H), 4.83 (s, 2H), 2.61 (d, J=12.0 Hz, 2H), 1.90-1.82 (m, 1H), 1.62 (d, J=10.8 Hz, 1H), 1.53 (d, J=10.0 Hz, 1H), 1.49-1.41 (m, 2H), 1.39 (s, 9H).

### Step 3: Synthesis of Intermediate 1-5.

To a solution of Intermediate 1-3 (398.99 mg, 1.31 mmol, 1 eq) in DMF (10 mL) was added HOAt (177.84 mg, 1.31 mmol, 182.77 μL, 1 eq) and EDCI (751.42 mg, 3.92 mmol, 3 eq). Then Intermediate 1-4 (1 g, 1.31 mmol, 1 eq, HCl salt) and NMM (660.78 mg, 6.53 mmol, 718.24 μL, 5 eq) was added into the system. The mixture was stirred at 25° C. for 1 hr. The reaction mixture was diluted with H₂O (5 mL) and extracted with EA (10 mL*3). The combined organic layers were washed with brine (10 mL*3), dried over Na₂SO₄, filtered and concentrated under reduced pressure to give a residue. The residue was purified by flash silica gel chromatography (ISCO; 20 g SepaFlash Silica Flash Column, Eluent of 0˜15% Methanol/Ethyl acetate gradient @60 mL/min), the eluent was concentrated under reduced pressure to give Intermediate 1-5 (760 mg, 729.99 μmol, 55.87% yield, 97.612% purity) as a yellow solid. LCMS (Method D): Retention time: 0.388 min, (M+H)⁺=1016.5. SFC: Retention time: 1.207, 1.342 min. ¹H NMR (400 MHz, CHLOROFORM-d) δ=10.21-9.86 (m, 1H), 8.51-8.43 (m, 1H), 7.78 (s, 2H), 7.70 (s, 2H), 7.66 (s, 1H), 7.39-7.36 (m, 2H), 7.32 (d, J=5.2 Hz, 2H), 7.11-6.98 (m, 2H), 5.05 (d, J=6.4 Hz, 1H), 4.29 (s, 2H), 4.23-4.11 (m, 2H), 3.85-3.54 (m, 12H), 3.34-3.14 (m, 4H), 2.96-2.67 (m, 6H), 2.49-2.38 (m, 4H), 2.20 (d, J=6.4 Hz, 2H), 2.14-1.96 (m, 4H), 1.76 (s, 3H), 1.48 (s, 9H), 1.28-1.09 (m, 9H), 1.03-0.94 (m, 3H).

### Step 4: Synthesis of Intermediate 1-6.

To a solution of Intermediate 1-5 (200 mg, 196.80 μmol, 1 eq) in DCM (2 mL) was added HCl/dioxane (2 M, 2 mL). The mixture was stirred at 25° C. for 0.5 hr. The aqueous layer was adjusted pH to 7-8 with ammonium hydroxide, and the reaction mixture was diluted with H₂O (5 mL) and extracted with EA (5 mL*3). The combined organic layers were dried over Na₂SO₄, filtered and concentrated under reduced pressure to give a residue. The crude product was used in the next step without further purification. Intermediate 1-6 (120 mg, 56.36 μmol, 28.64% yield, 44.7% purity, HCl salt) was obtained as a pale yellow solid. LCMS (Method D): Retention time: 0.276 min, (M+H)⁺=916.4.

### Step 5: Synthesis of I-352

To a solution of Intermediate 1-7 (13.66 mg, 104.98 μmol, 1 eq) in DMF (1 mL) was added HOAt (14.29 mg, 104.98 μmol, 14.68 μL, 1 eq) and EDCI (60.37 mg, 314.93 μmol, 3 eq). Then Intermediate 1-6 (100 mg, 104.98 μmol, 1 eq, HCl salt) and NMM (53.09 mg, 524.88 μmol, 57.71 μL, 5 eq) was added. The mixture was stirred at 25° C. for 1 hr. The reaction mixture was diluted with H₂O (5 mL) and extracted with EA (5 mL*3). The combined organic layers were washed with brine (5 mL*3), dried over Na₂SO₄, filtered and concentrated under reduced pressure to give a residue. The residue was purified by prep-HPLC (FA condition), the eluent was concentrated and lyophilized to give I-352 (24.90 mg, 22.93 μmol, 21.84% yield, 98.9% purity, FA salt) as a white solid. LCMS (Method D): Retention time: 0.350 min, (M+H)⁺=1028.5. SFC: Retention time: 3.749, 4.229 min. ¹H NMR (400 MHz, METHANOL-d4) δ=8.49 (s, 1H), 8.37 (d, J=8.0 Hz, 1H), 7.95 (d, J=7.2 Hz, 1H), 7.91-7.80 (m, 2H), 7.76 (s, 1H), 7.71 (d, J=7.6 Hz, 1H), 7.49 (d, J=6.0 Hz, 2H), 7.46-7.33 (m, 2H), 7.20-7.12 (m, 1H), 4.90 (d, J=8.8 Hz, 1H), 4.60-4.52 (m, 1H), 4.39 (s, 2H), 4.31-4.11 (m, 2H), 4.05-3.93 (m, 1H), 3.90-3.78 (m, 2H), 3.77-3.63 (m, 5H), 3.61-3.46 (m, 5H), 3.43-3.32 (m, 4H), 3.21-3.04 (m, 3H), 2.92-2.67 (m, 2H), 2.58-2.31 (m, 6H), 2.29-2.18 (m, 2H), 2.05 (d, J=12.0 Hz, 1H), 1.88 (d, J=12.0 Hz, 6H), 1.80-1.57 (m, 6H), 1.41-1.18 (m, 5H), 1.17-1.01 (m, 3H), 0.60-0.45 (m, 2H), 0.9-0.15 (m, 2H). ¹⁹F NMR (376 MHz, METHANOL-d4) δ=−120.77.

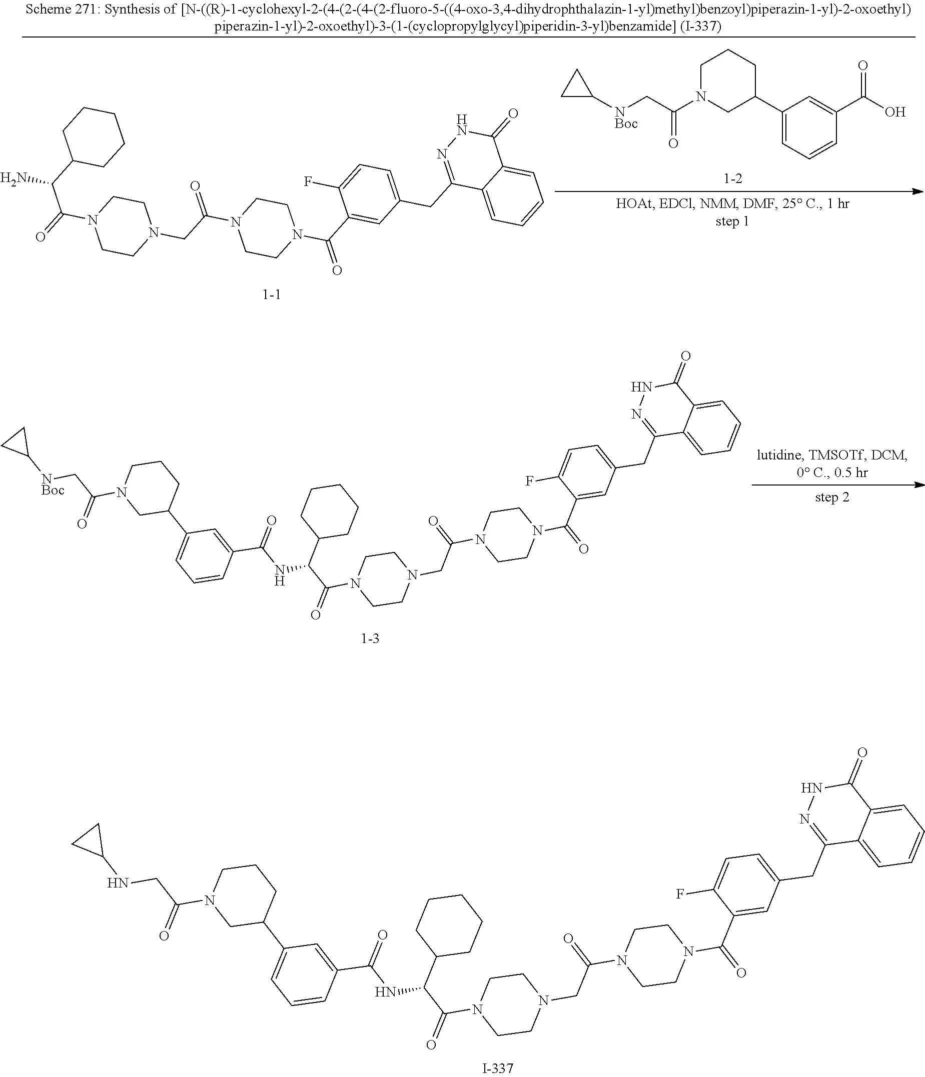 [SMILES: N[C@@H](C(=O)N1CCN(CC(=O)N2CCN(C(=O)c3cc(Cc4n[nH]c(=O)c5ccccc45)ccc3F)CC2)CC1)C1CCCCC1 ; CC(C)(C)OC(=O)N(CC(=O)N1CCCC(c2cccc(C(=O)O)c2)C1)C1CC1 ; CC(C)(C)OC(=O)N(CC(=O)N1CCCC(c2cccc(C(=O)N[C@@H](C(=O)N3CCN(CC(=O)N4CCN(C(=O)c5cc(Cc6n[nH]c(=O)c7ccccc67)ccc5F)CC4)CC3)C3CCCCC3)c2)C1)C1CC1 ; O=C(N[C@@H](C(=O)N1CCN(CC(=O)N2CCN(C(=O)c3cc(Cc4n[nH]c(=O)c5ccccc45)ccc3F)CC2)CC1)C1CCCCC1)c1cccc(C2CCCN(C(=O)CNC3CC3)C2)c1]

### Step 1: Synthesis of Intermediate 1-3.

To a solution of Intermediate 1-2 (90.35 mg, 224.48 μmol, 1 eq) and Intermediate 1-1 (150 mg, 224.48 μmol, 1 eq, HCl salt) in DMF (1 mL) was added HOAt (30.55 mg, 224.48 μmol, 31.40 μL, 1 eq) and EDCI (129.10 mg, 673.45 μmol, 3 eq) and NMM (113.53 mg, 1.12 mmol, 123.40 μL, 5 eq). The mixture was stirred at 25° C. for 1 hr. The reaction mixture was diluted with H₂O (2 mL) and extracted with EA (5 mL*3). The combined organic layers were washed with brine (5 mL*3), dried over Na₂SO₄, filtered and concentrated under reduced pressure to give a residue. The residue was purified by flash silica gel chromatography (ISCO; 20 g SepaFlash Silica Flash Column, Eluent of 0˜15% Methanol/Ethyl acetate gradient @60 mL/min), the eluent was concentrated under reduced pressure to give Intermediate 1-3 (130 mg, 127.13 μmol, 56.63% yield, 99.3% purity) as a yellow solid. LCMS (Method D): Retention time: 0.426 min, (M+H)⁺=1016.4. SFC: Retention time: 0.515 min, 0.880 min. ¹H NMR (400 MHz, METHANOL-d4) δ=8.36 (d, J=7.2 Hz, 1H), 7.98-7.91 (m, 1H), 7.90-7.79 (m, 2H), 7.78-7.68 (m, 2H), 7.54-7.33 (m, 4H), 7.21-7.12 (m, 1H), 4.89 (s, 1H), 4.61-4.51 (m, 2H), 4.38 (s, 2H), 4.22-4.11 (m, 1H), 4.10-3.98 (m, 1H), 3.95-3.82 (m, 2H), 3.82-3.67 (m, 6H), 3.55 (d, J=18.8 Hz, 4H), 3.26 (s, 1H), 3.22-3.09 (m, 1H), 2.85-2.67 (m, 3H), 2.62-2.43 (m, 4H), 2.05 (d, J=8.0 Hz, 1H), 1.95-1.83 (m, 4H), 1.78 (d, J=10.8 Hz, 2H), 1.69 (d, J=9.6 Hz, 3H), 1.45 (s, 9H), 1.38-1.18 (m, 4H), 1.16-1.05 (m, 2H), 0.75-0.57 (m, 4H).

### Step 2: Synthesis of I-337

To a solution of Intermediate 1-3 (100 mg, 98.41 μmol, 1 eq) in DCM (1 mL) was added 2,6-dimethylpyridine (3.16 mg, 29.52 μmol, 3.44 μL, 0.3 eq) and trimethylsilyl trifluoromethanesulfonate (32.81 mg, 147.61 μmol, 26.67 μL, 1.5 eq). The mixture was stirred at 0° C. for 0.5 hr. The pH of the aqueous layer was adjusted to 7-8 with ammonium hydroxide, the reaction mixture was concentrated under reduced pressure to give a residue. The residue was purified by prep-HPLC (NH₄HCO₃ condition), the eluent was concentrated and lyophilized to give I-337 (25.45 mg, 27.78 μmol, 28.23% yield, 100% purity). LCMS (Method D): Retention time: 0.354 min, (M+H)⁺=916.5. SFC: Retention time: 6.415, 7.756 min. ¹H NMR (400 MHz, METHANOL-d4) δ=8.36 (d, J=7.2 Hz, 1H), 7.98-7.92 (m, 1H), 7.89-7.79 (m, 2H), 7.78-7.68 (m, 2H), 7.51-7.35 (m, 4H), 7.20-7.11 (m, 1H), 4.90 (s, 1H), 4.58 (d, J=10.0 Hz, 1H), 4.38 (s, 2H), 3.97-3.85 (m, 2H), 3.83-3.64 (m, 7H), 3.63-3.44 (m, 6H), 3.25 (s, 1H), 3.22-3.08 (m, 1H), 2.88-2.69 (m, 2H), 2.64-2.43 (m, 4H), 2.28-2.19 (m, 1H), 2.03 (d, J=2.8 Hz, 1H), 1.96-1.82 (m, 4H), 1.80-1.73 (m, 2H), 1.69 (d, J=11.2 Hz, 3H), 1.38-1.16 (m, 4H), 1.15-1.04 (m, 2H), 0.50-0.43 (m, 2H), 0.42-0.34 (m, 2H). ¹⁹F NMR (376 MHz, METHANOL-d4) δ=−120.74.

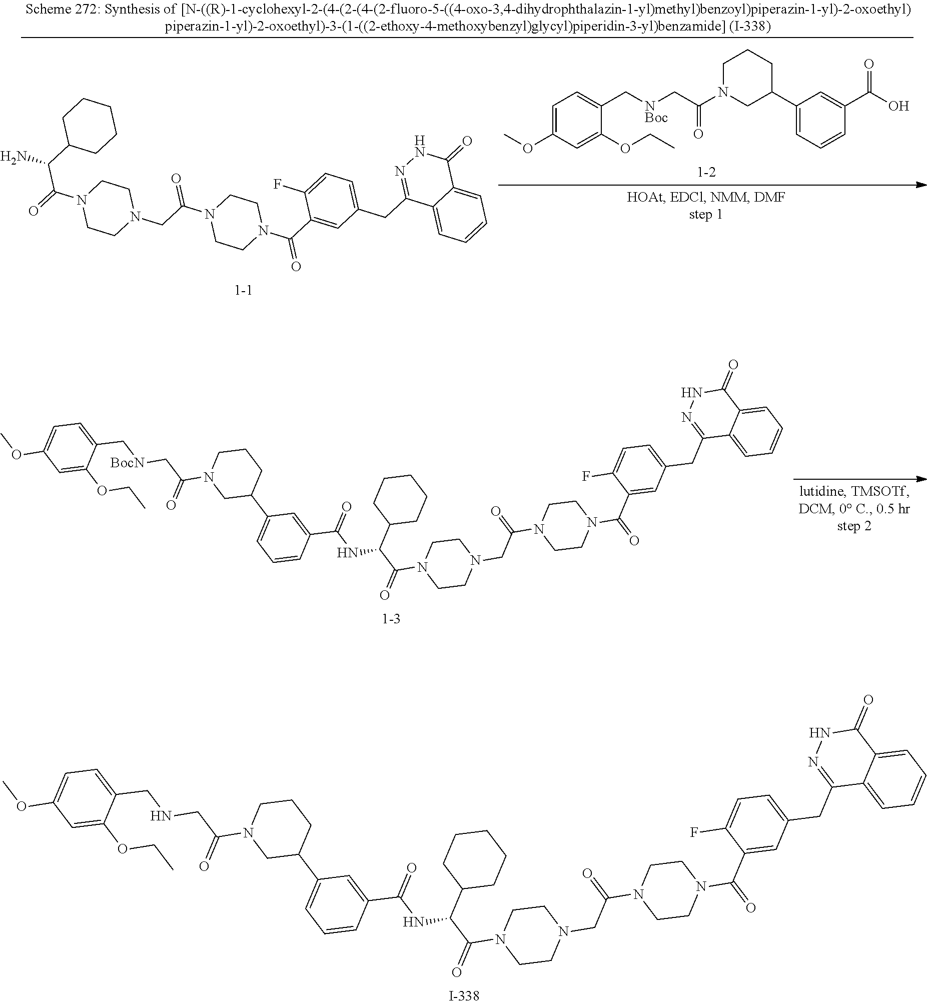

### Step 1: Synthesis of Intermediate 1-3.

To a solution of Intermediate 1-2 (157.62 mg, 299.31 μmol, 1 eq) and Intermediate 1-1 (200 mg, 299.31 μmol, 1 eq, HCl salt) in DMF (2 mL) was added HOAt (40.74 mg, 299.31 μmol, 41.87 μL, 1 eq) and EDCI (172.14 mg, 897.94 μmol, 3 eq) and NMM (151.37 mg, 1.50 mmol, 164.54 μL, 5 eq). The mixture was stirred at 25° C. for 1 hr. The reaction mixture diluted with H₂O (30 mL) and filtered to give a residue. The residue was purified by flash silica gel chromatography (ISCO; 20 g SepaFlash Silica Flash Column, Eluent of 0˜20% Methanol/Ethyl acetate gradient @60 mL/min), the eluent was concentrated under reduced pressure to give Intermediate 1-3 (230 mg, 199.35 μmol, 66.60% yield, 98.8% purity) as a white solid. LCMS (Method D): Retention time: 0.457 min, (M+H)⁺=690.5. SFC: Retention time: 1.970 min, 2.090 min. ¹H NMR (400 MHz, METHANOL-d4) δ=8.40-8.32 (m, 1H), 7.97-7.92 (m, 1H), 7.90-7.80 (m, 2H), 7.77-7.65 (m, 2H), 7.51-7.33 (m, 4H), 7.21-7.06 (m, 2H), 6.53-6.43 (m, 2H), 4.91 (d, J=8.8 Hz, 1H), 4.61-4.53 (m, 1H), 4.46-4.34 (m, 4H), 4.16-3.97 (m, 4H), 3.92-3.64 (m, 12H), 3.54 (d, J=18.0 Hz, 3H), 3.35 (s, 2H), 3.25 (s, 1H), 3.18-3.02 (m, 1H), 2.80-2.66 (m, 2H), 2.64-2.43 (m, 4H), 2.07-1.99 (m, 1H), 1.94-1.81 (m, 4H), 1.80-1.73 (m, 2H), 1.69 (d, J=11.2 Hz, 2H), 1.64-1.54 (m, 1H), 1.46 (d, J=6.8 Hz, 9H), 1.42-1.33 (m, 3H), 1.32-1.20 (m, 3H), 1.16-1.04 (m, 2H). ¹⁹F NMR (376 MHz, CHLOROFORM-d) δ=−120.747.

### Step 2: Synthesis of I-338

To a solution of Intermediate 1-3 (200 mg, 175.39 μmol, 1 eq) in DCM (2 mL) was added 2,6-dimethylpyridine (5.64 mg, 52.62 μmol, 6.13 μL, 0.3 eq) and trimethylsilyl trifluoromethanesulfonate (58.47 mg, 263.08 μmol, 47.54 μL, 1.5 eq). The mixture was stirred at 0° C. for 0.5 hr. The pH of water layers adjusted to 7-8 with ammonium hydroxide, the reaction mixture was concentrated under reduced pressure to give a residue. The residue was purified by prep-HPLC (NH₃·H₂O condition), the eluent was concentrated and lyophilized to give I-338 (56.46 mg, 54.28 μmol, 30.95% yield, 100% purity) as a white solid. LCMS (Method D): Retention time: 0.374 min, (M+H)⁺=1040.5. ¹H NMR (400 MHz, METHANOL-d4) δ=8.36 (d, J=6.4 Hz, 1H), 7.97-7.90 (m, 1H), 7.89-7.78 (m, 2H), 7.72 (d, J=13.2 Hz, 2H), 7.51-7.31 (m, 4H), 7.20-7.08 (m, 2H), 6.54-6.39 (m, 2H), 4.90 (s, 1H), 4.51 (s, 1H), 4.37 (s, 2H), 4.09-4.01 (m, 2H), 3.93-3.78 (m, 4H), 3.78-3.69 (m, 9H), 3.68-3.64 (m, 1H), 3.60-3.50 (m, 3H), 3.47-3.39 (m, 2H), 3.35 (s, 1H), 3.25 (s, 1H), 3.18-3.03 (m, 1H), 2.79-2.60 (m, 3H), 2.57 (s, 3H), 2.00 (d, J=11.2 Hz, 1H), 1.94-1.85 (m, 3H), 1.78 (d, J=10.0 Hz, 3H), 1.69 (d, J=9.2 Hz, 2H), 1.62-1.49 (m, 1H), 1.47-1.37 (m, 3H), 1.36-1.15 (m, 4H), 1.15-1.04 (m, 2H). ¹⁹F NMR (376 MHz, DMSO-d6) δ=−120.693.

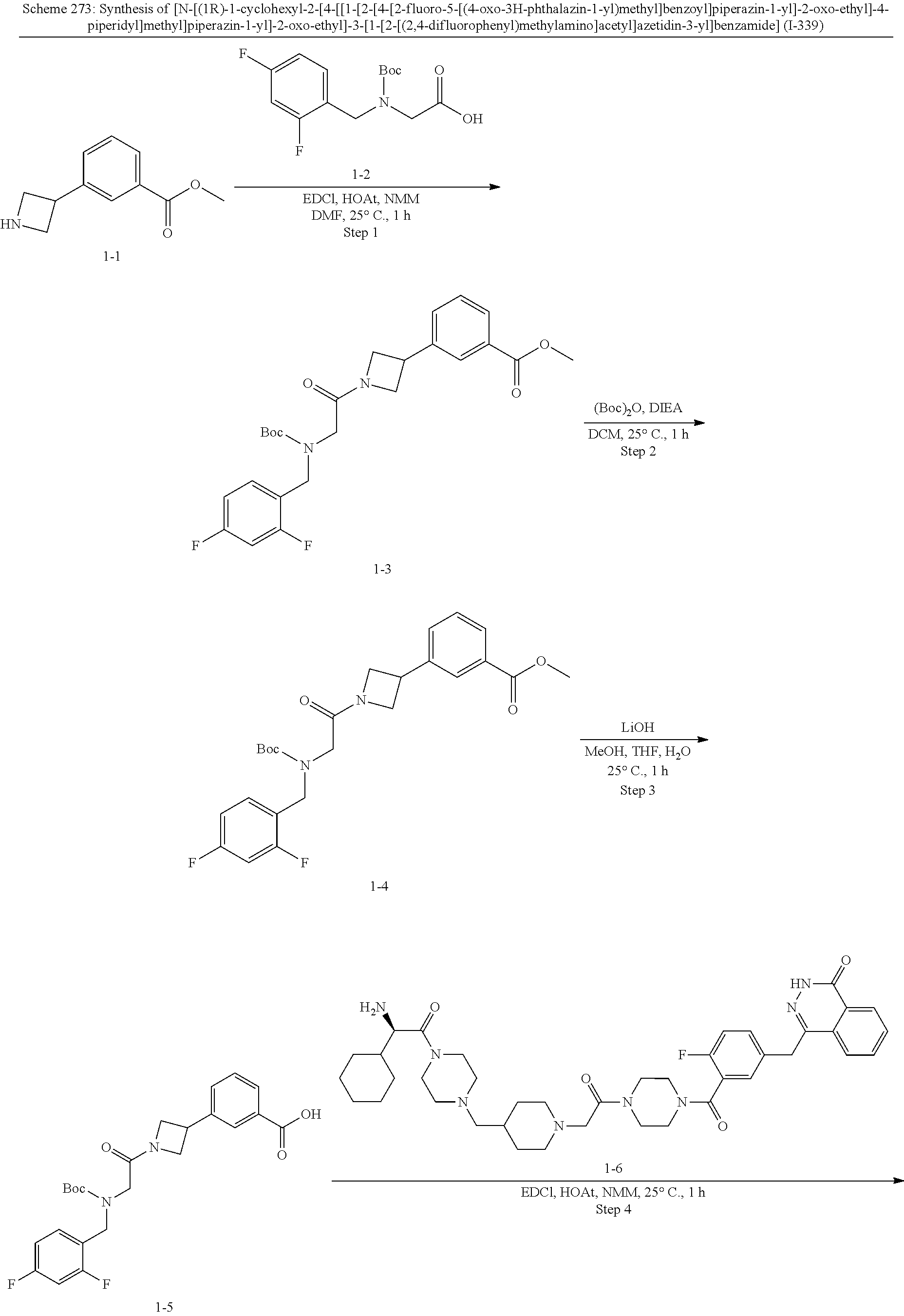

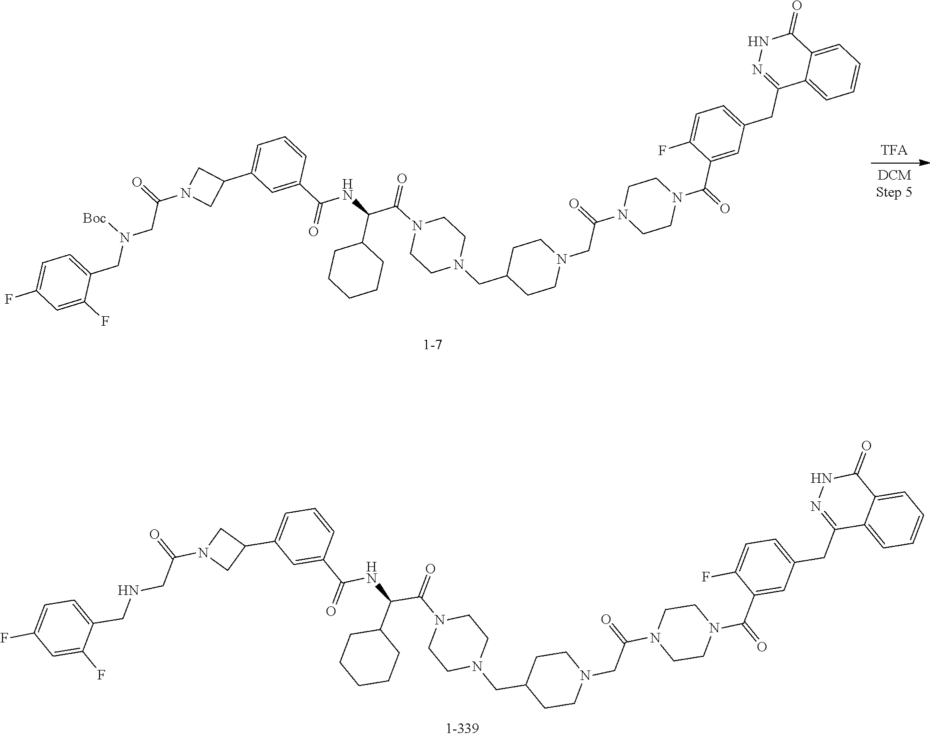 [SMILES: CC(C)(C)OC(=O)N(CC(=O)N1CC(c2cccc(C(=O)N[C@@H](C(=O)N3CCN(CC4CCN(CC(=O)N5CCN(C(=O)c6cc(Cc7n[nH]c(=O)c8ccccc78)ccc6F)CC5)CC4)CC3)C3CCCCC3)c2)C1)Cc1ccc(F)cc1F ; O=C(N[C@@H](C(=O)N1CCN(CC2CCN(CC(=O)N3CCN(C(=O)c4cc(Cc5n[nH]c(=O)c6ccccc56)ccc4F)CC3)CC2)CC1)C1CCCCC1)c1cccc(C2CN(C(=O)CNCc3ccc(F)cc3F)C2)c1]

### Step 1: Synthesis of Intermediate 1-3.

To a mixture of intermediate 1-1 (180 mg, 589.68 μmol, 1 eq, TFA salt) in DMF (2 mL) was added intermediate 1-2 (195.43 mg, 648.65 μmol, 1.1 eq), EDCI (339.13 mg, 1.77 mmol, 3 eq), HOAt (80.26 mg, 589.68 μmol, 82.49 μL, 1 eq) and NMM (298.22 mg, 2.95 mmol, 324.16 μL, 5 eq) at 25° C. The mixture was stirred at 25° C. for 1 hr. The reaction mixture was diluted with H₂O (3 mL), extracted with EtOAc (2 mL×3), dried over Na₂SO₄, filtered and concentrated to give a residue. The residue was purified by column chromatography (SiO₂, EA/MeOH=1/0 to 7/3) and concentrated to give Intermediate 1-3 (150 mg, 400.66 μmol, 67.95% yield) as a yellow oil. LCMS (method D): Retention time: 0.268 min, (M+1)⁺=375.1. ¹H NMR (400 MHz, METHANOL-d4) δ=8.02 (s, 1H), 7.94 (d, J=8.0 Hz, 1H), 7.62 (d, J=7.2 Hz, 1H), 7.55-7.41 (m, 2H), 6.96 (t, J=8.8 Hz, 2H), 4.67-4.57 (m, 1H), 4.49-4.40 (m, 1H), 4.26-4.20 (m, 1H), 4.07-3.98 (m, 2H), 3.92 (s, 3H), 3.87-3.82 (m, 2H), 3.34 (s, 2H).

### Step 2: Synthesis of Intermediate 1-4.

To a mixture of intermediate 1-3 (150 mg, 400.66 μmol, 1 eq) in DCM (1.5 mL) was added (Boc)₂O (104.93 mg, 480.79 μmol, 110.45 μL, 1.2 eq) and DIEA (77.67 mg, 600.99 μmol, 104.68 μL, 1.5 eq) at 25° C., and the mixture was stirred at 25° C. for 1 hr. The reaction mixture was diluted with H₂O (2 mL) and extracted with DCM (2 mL×3), the combined organic layers were dried over Na₂SO₄, filtered and concentrated to give a residue. The residue was purified by column chromatography (SiO₂, PE/EA=1/0 to 3/7) and concentrated to give Intermediate 1-4 (110 mg, 231.82 μmol, 57.86% yield) as a colorless oil. LCMS (Method D): Retention time: 0.440 min, (M+Na)⁺=497.1.

### Step 3: Synthesis of Intermediate 1-5.

To a mixture of intermediate 1-4 (100 mg, 210.75 μmol, 1 eq) in H₂O (0.5 mL), THF (0.5 mL) and MeOH (0.5 mL) was added LiOH·H₂O (26.53 mg, 632.25 μmol, 3 eq) at 25° C., and the mixture was stirred at 25° C. for 1 hr. The reaction mixture was diluted with H₂O (2 mL) and extracted with DCM (1 mL×3), the combined organic layers were dried over Na₂SO₄, filtered and concentrated to give the crude product. The crude product was used for the next step directly. Intermediate 1-5 (68 mg, 147.68 μmol, 70.07% yield) was obtained as a white solid. LCMS (Method D): Retention time: 0.402 min, (M+Na*)=483.1.

### Step 4: Synthesis of Intermediate 1-7.

To a mixture of intermediate 1-5 (55 mg, 119.44 μmol, 1 eq) in DMF (0.5 mL) was added intermediate 1-6 (100.56 mg, 131.39 μmol, 1.1 eq, HCl salt), EDCI (68.69 mg, 358.33 μmol, 3 eq), HOAt (16.26 mg, 119.44 μmol, 16.71 μL, 1 eq) and NMM (60.41 mg, 597.22 μmol, 65.66 μL, 5 eq) at 25° C., the mixture was stirred at 25° C. for 1 hr. The reaction mixture was concentrated to give a residue. The residue was purified by reversed-phase HPLC (0.1% FA condition) and lyophilized to give Intermediate 1-7 (80 mg, 68.30 μmol, 57.18% yield) as a yellow solid. LCMS (Method D): Retention time: 0.432 min, (M+H)⁺=1171.6.

### Step 5: Synthesis of I-339

To a mixture of intermediate 1-7 (20 mg, 17.07 μmol, 1 eq) in DCM (0.5 mL) was added TFA (153.50 mg, 1.35 mmol, 0.1 mL, 78.84 eq) at 25° C., and the mixture was stirred at 25° C. for 0.5 hr. The reaction mixture was concentrated to give a residue. The residue was purified by reversed-phase HPLC (0.1% TFA condition) and lyophilized to give I-339 (4.47 mg, 3.67 μmol, 21.49% yield, 97.2% purity, TFA salt) as a yellow solid. LCMS (Method D): Retention time: 0.432 min, (M+H)⁺=1171.6. SFC: Retention time: 0.926 min. ¹H NMR (400 MHz, METHANOL-d4) δ=8.56-8.48 (m, 1H), 8.41-8.33 (m, 1H), 7.98-7.92 (m, 1H), 7.90-7.76 (m, 3H), 7.64-7.57 (m, 2H), 7.54-7.46 (m, 2H), 7.41-7.07 (m, 4H), 4.69-4.48 (m, 3H), 4.39 (s, 2H), 4.33 (s, 2H), 4.31-4.25 (m, 2H), 4.24-4.19 (m, 1H), 4.19-4.04 (m, 3H), 3.95 (s, 3H), 3.84-3.76 (m, 2H), 3.75-3.61 (m, 3H), 3.60-3.44 (m, 3H), 3.40-3.33 (m, 4H), 3.17-3.00 (m, 4H), 2.99-2.76 (m, 3H), 2.24-2.03 (m, 3H), 2.01-1.86 (m, 2H), 1.85-1.56 (m, 6H), 1.40-1.05 (m, 6H). ¹⁹F NMR (376 MHz, METHANOL-d4) δ=−108.2, δ=−112.9, δ=−120.7.

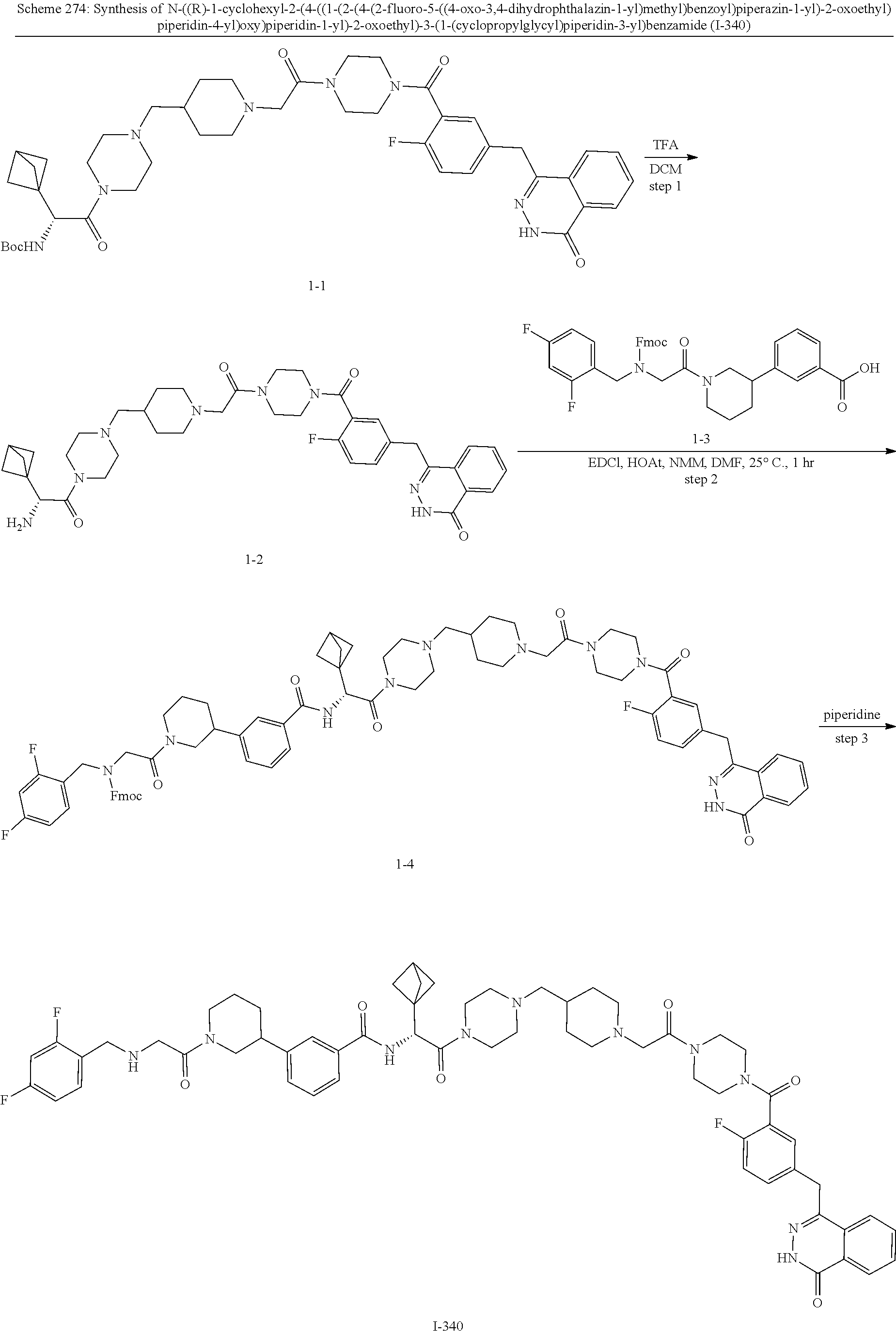

### Step 1: Synthesis of Intermediate 1-2

To a solution of Intermediate 1-1 (100 mg, 123.01 μmol, 1 eq) in DCM (1 mL) was added TFA (307.00 mg, 2.69 mmol, 0.2 mL, 21.89 eq). The mixture was stirred at 25° C. for 1 hr. The reaction mixture was concentrated under reduced pressure to give Intermediate 1-2 (100 mg, 120.94 μmol, 98.32% yield, TFA salt) as a white solid. LCMS (Method D): Retention time: 0.206 min, (M+H)⁺=713.3.

### Step 2: Synthesis of Intermediate 1-4

To a solution of Intermediate 1-2 (100 mg, 120.94 μmol, 1 eq, TFA salt) and Intermediate 1-3 (59.08 mg, 120.94 μmol, 1 eq) in DMF (1 mL) was added EDCI (46.37 mg, 241.87 μmol, 2 eq) and HOAt (16.46 mg, 120.94 μmol, 16.92 μL, 1 eq) and NMM (61.16 mg, 604.68 μmol, 66.48 μL, 5 eq). The mixture was stirred at 25° C. for 1 hr. The reaction mixture was diluted with MeOH. The crude product was purified by reversed-phase HPLC (0.1% FA condition), and the eluent was concentrated to remove ACN and lyophilized to get Intermediate 1-4 (40 mg, 33.80 μmol, 27.95% yield) as a white solid. LCMS (Method D): Retention time: 0.399 min, (M+H)⁺=1183.6.

### Step 3: Synthesis of I-340

To a solution of Intermediate 1-4 (30 mg, 25.35 μmol, 1 eq) in DCM (0.2 mL) was added HCl/dioxane (2 M, 0.2 mL, 15.78 eq). The mixture was stirred at 25° C. for 1 hr. The reaction mixture was concentrated under reduced pressure to give a residue. The residue was purified by reversed-phase HPLC (0.1% FA condition), the eluent was concentrated to remove ACN and lyophilized to give I-340 (13.37 mg, 11.79 μmol, 46.51% yield, 99.5% purity, FA salt) as a white solid. LCMS (Method D): Retention time: 0.317 min, (M+Na)⁺=1105.6. SFC: Retention time: 4.862, 5.546 min. ¹H NMR (400 MHz, METHANOL-d4) δ=8.43 (s, 1H), 8.37 (d, J=7.6 Hz, 1H), 8.01-7.92 (m, 1H), 7.91-7.79 (m, 2H), 7.79-7.67 (m, 2H), 7.59-7.42 (m, 4H), 7.37 (t, J=7.2 Hz, 1H), 7.17 (t, J=9.2 Hz, 1H), 7.05-7.01 (m, 2H), 5.24 (d, J=3.6 Hz, 1H), 4.59-4.48 (m, 1H), 4.39 (s, 2H), 4.04-3.96 (m, 2H), 3.85-3.67 (m, 10H), 3.64-3.37 (m, 6H), 3.27-3.13 (m, 2H), 3.04-2.35 (m, 10H), 2.29 (t, J=6.8 Hz, 2H), 2.10-2.02 (m, 1H), 2.00-1.53 (m, 13H), 1.51-1.35 (m, 2H).

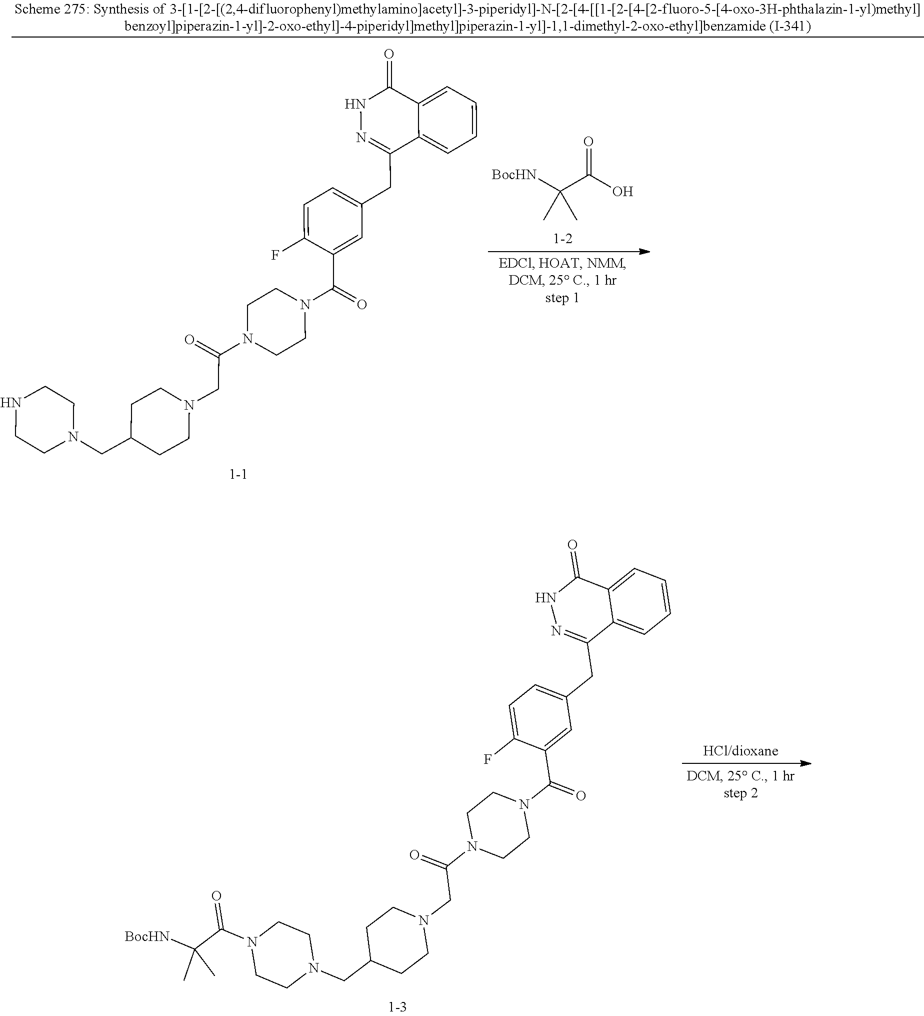 [SMILES: O=C(CN1CCC(CN2CCNCC2)CC1)N1CCN(C(=O)c2cc(Cc3n[nH]c(=O)c4ccccc34)ccc2F)CC1 ; CC(C)(C)OC(=O)NC(C)(C)C(=O)O ; CC(C)(C)OC(=O)NC(C)(C)C(=O)N1CCN(CC2CCN(CC(=O)N3CCN(C(=O)c4cc(Cc5n[nH]c(=O)c6ccccc56)ccc4F)CC3)CC2)CC1]

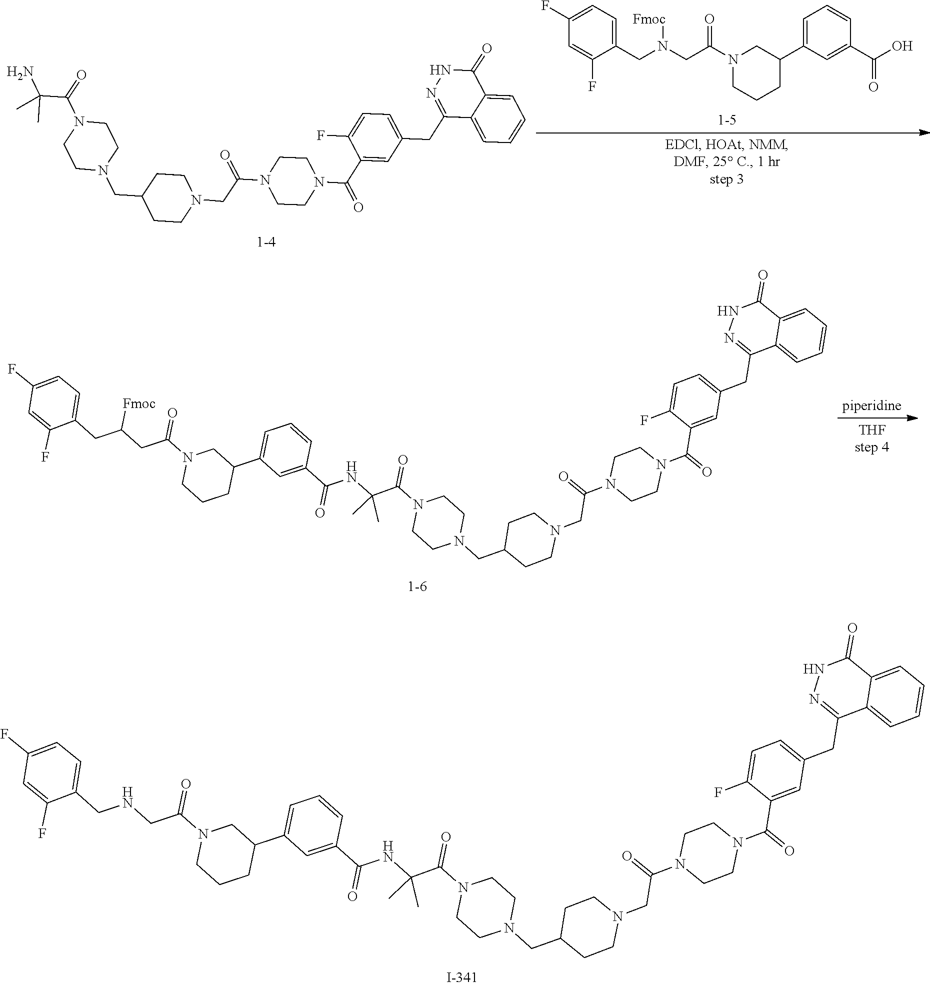

### Step 1: Synthesis of Intermediate 1-3

To a solution of Intermediate 1-2 (172.32 mg, 847.89 μmol, 1 eq) in DCM (5 mL) was added EDCI (487.62 mg, 2.54 mmol, 3 eq), HOAt (115.41 mg, 847.89 μmol, 118.61 μL, 1 eq) and NMM (428.81 mg, 4.24 mmol, 466.09 μL, 5 eq) at 25° C. for 0.5 hr. Then Intermediate 1-1 (500 mg, 847.89 μmol, 1 eq) was added into the mixture and it was stirred at 25° C. for 0.5 hr. The reaction mixture was diluted with water (60 mL) and extracted with DCM (20 mL×3). The combined organic layers were washed with brine (20 mL×3) and dried over sodium sulfate, filtered and concentrated under reduced pressure to give a residue. The crude product was purified by reversed-phase HPLC (H₂O condition) and the eluent was concentrated under reduced pressure to remove ACN and then lyophilized to give Intermediate 1-3 (300 mg, 379.39 μmol, 44.75% yield, 98% purity) as a yellow oil LCMS (Method D): Rt: 0.272 min, (M+H)⁺=775.5.

### Step 2: Synthesis of Intermediate 1-4

To a solution of Intermediate 1-3 (300 mg, 387.14 μmol, 1 eq) in DCM (3 mL) was added HCl/dioxane (2 M, 580.70 μL, 3 eq). The mixture was stirred at 25° C. for 0.5 hr. The eluent was concentrated under reduced pressure to give Intermediate 1-4 (220 mg, 326.02 μmol, 84.21% yield) as a yellow oil. LCMS (Method D): Rt: 0.215 min, (M+H)⁺=675.3.

### Step 3: Synthesis of Intermediate 1-6

To a solution of Intermediate 1-5 (199.08 mg, 326.02 μmol, 1 eq) in DMF (2.2 mL) was added EDCI (187.49 mg, 978.06 μmol, 3 eq), HOAt (44.37 mg, 326.02 μmol, 45.61 μL, 1 eq) and NMM (164.88 mg, 1.63 mmol, 179.22 μL, 5 eq) at 25° C. for 0.5 hr. Then Intermediate 1-4 (220 mg, 326.02 μmol, 1 eq) was added into the mixture and it was stirred at 25° C. for 0.5 hr. The reaction mixture was diluted with water (18 mL) and extracted with DCM (6 mL×3). The combined organic layers were washed with brine (6 mL×3) and dried over sodium sulfate, filtered and concentrated under reduced pressure to give a residue. The crude product was purified by reversed-phase HPLC (0.1% FA condition) and the eluent was concentrated under reduced pressure to remove ACN and then lyophilized to give Intermediate 1-6 (200 mg, 154.17 μmol, 47.29% yield, 97.7% purity) as a white solid. LCMS (Method D): Rt: 0.418 min, (M+H)⁺=1268.2.

### Step 4: Synthesis of I-341

To a solution of Intermediate 1-6 (200 mg, 157.80 μmol, 1 eq) in THF (2 mL) was added piperidine (230.56 mg, 2.71 mmol, 267.41 μL, 17.16 eq). The mixture was stirred at 25° C. for 1 hr. The reaction mixture was diluted with water (9 mL) and extracted with ethyl acetate (3 mL×3). The combined organic layers were washed with brine (3 mL×3) and dried over sodium sulfate, filtered and concentrated under reduced pressure to give a residue. The residue was purified by prep-HPLC (neutral condition). The eluent was concentrated under reduced pressure to remove ACN and then lyophilized to give I-341 (44.27 mg, 40.36 μmol, 25.58% yield, 99.48% purity, FA salt) as a white solid. LCMS (Method D): Rt: 0.281 min, (M+H)⁺=1045.8. ¹H NMR (400 MHz, METHANOL-d4) δ=8.34 (d, J=7.6 Hz, 1H), 7.96-7.90 (m, 1H), 7.89-7.77 (m, 2H), 7.75-7.65 (m, 2H), 7.52-7.39 (m, 4H), 7.36 (s, 1H), 7.16-7.14 (m, 1H), 7.01-6.83 (m, 2H), 4.56 (d, J=10.4 Hz, 1H), 4.36 (s, 2H), 3.91-3.77 (m, 4H), 3.75 (s, 1H), 3.74-3.61 (m, 6H), 3.57-3.44 (m, 4H), 3.28 (s, 1H), 3.23-3.04 (m, 3H), 2.90-2.66 (m, 4H), 2.33 (s, 4H), 2.12-2.11 (m, 2H), 2.06-1.95 (m, 3H), 1.84-1.83 (m, 2H), 1.76-1.38 (m, 11H), 1.32-1.07 (m, 2H). ¹⁹F NMR (376 MHz, METHANOL-d4) δ=−113.326, −16.186, −120.629, −120.704. SFC: Rt: 4.122, 4.872 min.

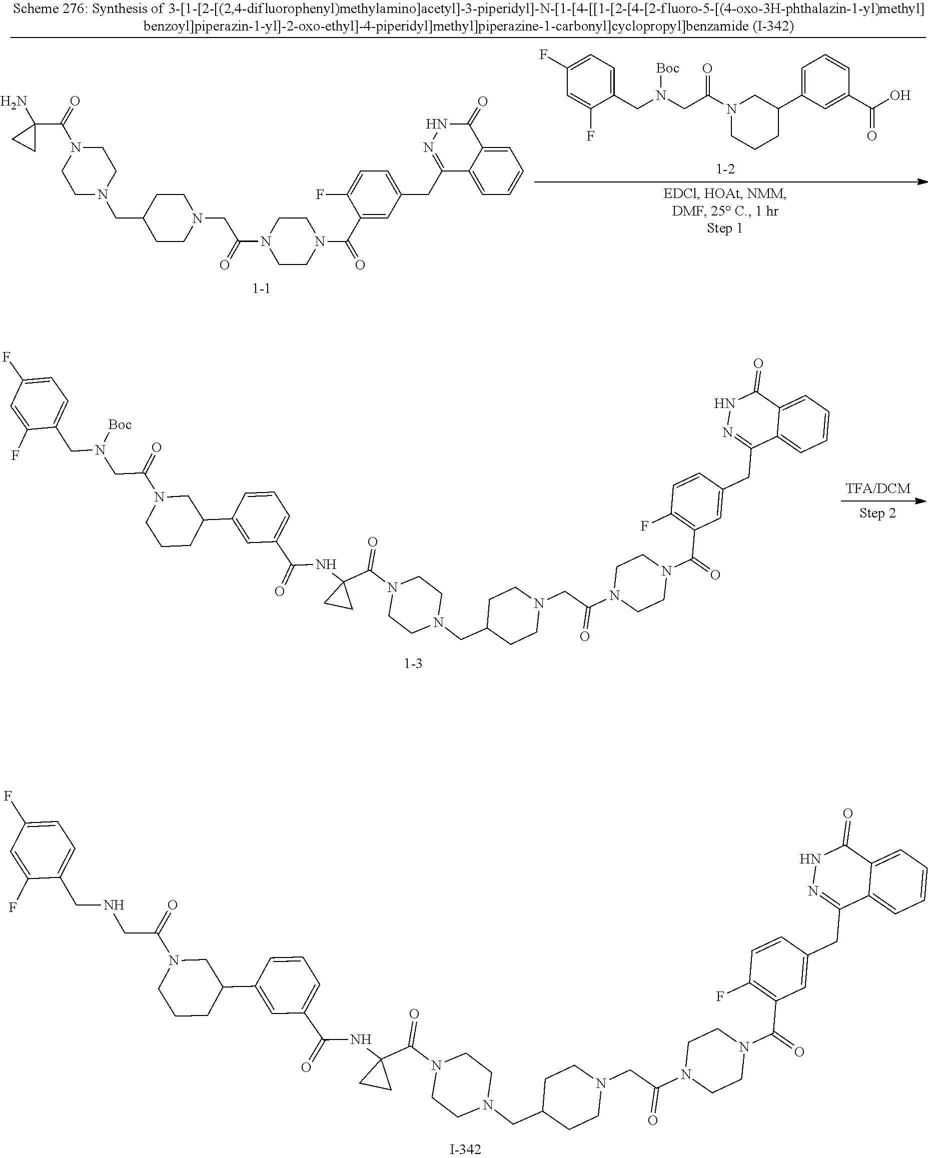 [SMILES: NC1(C(=O)N2CCN(CC3CCN(CC(=O)N4CCN(C(=O)c5cc(Cc6n[nH]c(=O)c7ccccc67)ccc5F)CC4)CC3)CC2)CC1 ; CC(C)(C)OC(=O)N(CC(=O)N1CCCC(c2cccc(C(=O)O)c2)C1)Cc1ccc(F)cc1F ; CC(C)(C)OC(=O)N(CC(=O)N1CCCC(c2cccc(C(=O)NC3(C(=O)N4CCN(CC5CCN(CC(=O)N6CCN(C(=O)c7cc(Cc8n[nH]c(=O)c9ccccc89)ccc7F)CC6)CC5)CC4)CC3)c2)C1)Cc1ccc(F)cc1F ; O=C(NC1(C(=O)N2CCN(CC3CCN(CC(=O)N4CCN(C(=O)c5cc(Cc6n[nH]c(=O)c7ccccc67)ccc5F)CC4)CC3)CC2)CC1)c1cccc(C2CCCN(C(=O)CNCc3ccc(F)cc3F)C2)c1]

### Step 1: Synthesis of Intermediate 1-3

To a solution of intermediate 1-1 (88.00 mg, 180.14 μmol, 1 eq) in DMF (0.5 mL) was added HOAt (24.52 mg, 180.14 μmol, 25.20 μL, 1 eq), NMM (91.10 mg, 900.69 μmol, 99.02 μL, 5 eq) and EDCI (103.60 mg, 540.41 μmol, 3 eq). The mixture was stirred at 25° C. for 0.5 hr. Then intermediate 1-2 (141.73 mg, 180.14 μmol, 1 eq, TFA salt) was added into the mixture and stirred at 25° C. for 0.5 hr. The reaction mixture was diluted with water (12 mL) and extracted with ethyl acetate (4 mL×3). The combined organic layers were washed with brine (4 mL×3) and dried over sodium sulfate, filtered and concentrated under reduced pressure to give a residue. The crude product was purified by reversed-phase HPLC (H₂O). The eluent was concentrated under reduced pressure to remove ACN and then lyophilized to give intermediate 1-3 (110 mg, 85.51 μmol, 47.47% yield, 88.88% purity) as a yellow solid. LCMS (Method D): Rt: 0.383 min, (M+H)⁺=1143.4. SFC: Rt: 2.863, 3.093 min. ¹H NMR (400 MHz, CHLOROFORM-d) δ=10.59-9.89 (m, 1H), 8.46 (d, J=8.4 Hz, 1H), 7.85-7.55 (m, 5H), 7.40-7.29 (m, 5H), 7.13-6.97 (m, 2H), 6.89-6.78 (m, 2H), 4.73-4.44 (m, 4H), 4.29 (s, 2H), 3.92-3.59 (m, 12H), 3.29-3.15 (m, 3H), 2.94-2.68 (m, 4H), 2.40 (s, 4H), 2.26-1.97 (m, 6H), 1.74-1.73 (m, 6H), 1.46 (s, 9H), 1.40-1.18 (m, 7H).

### Step 2: Synthesis of I-342

To a solution of intermediate 1-3 (110 mg, 96.21 μmol, 1 eq) in DCM (1 mL) was added TFA (307.00 mg, 2.69 mmol, 200 μL, 27.98 eq). The mixture was stirred at 25° C. for 1 hr. The mixture was adjusted to pH=7-8 with an aqueous solution of sodium bicarbonate. The reaction mixture was diluted with MeOH (2 mL). The crude product was purified by reversed-phase HPLC (0.1% NH₃·H₂O). The eluent was concentrated under reduced pressure to remove ACN and then lyophilized to give I-342 (31.80 mg, 29.84 μmol, 31.01% yield, 97.88% purity) as a white solid. LCMS (Method H): Rt: 0.581 min, (M+H)⁺=1043.4. SFC: Rt: 3.800, 4.487 min. ¹H NMR (400 MHz, METHANOL-d4) δ=8.36 (d, J=7.6 Hz, 1H), 7.99-7.91 (m, 1H), 7.90-7.75 (m, 2H), 7.74-7.63 (m, 2H), 7.52-7.40 (m, 4H), 7.36 (d, J=4.0 Hz, 1H), 7.16-7.14 (m, J=8.8 Hz, 1H), 7.00-6.86 (m, 2H), 4.56 (d, J=10.8 Hz, 1H), 4.38 (s, 2H), 3.91-3.74 (m, 5H), 3.68 (d, J=12.0 Hz, 6H), 3.56-3.45 (m, 4H), 3.28 (s, 2H), 3.21 (s, 1H), 3.19-3.05 (m, 2H), 2.97-2.83 (m, 2H), 2.80-2.66 (m, 2H), 2.36 (s, 4H), 2.20-1.99 (m, 5H), 1.90-1.79 (m, 2H), 1.77-1.66 (m, 2H), 1.62-1.47 (m, 2H), 1.39 (s, 2H), 1.29-1.09 (m, 4H). ¹⁹F NMR (400 MHz, METHAN OL-d4) δ=−113.280, −116.215, −120.800.

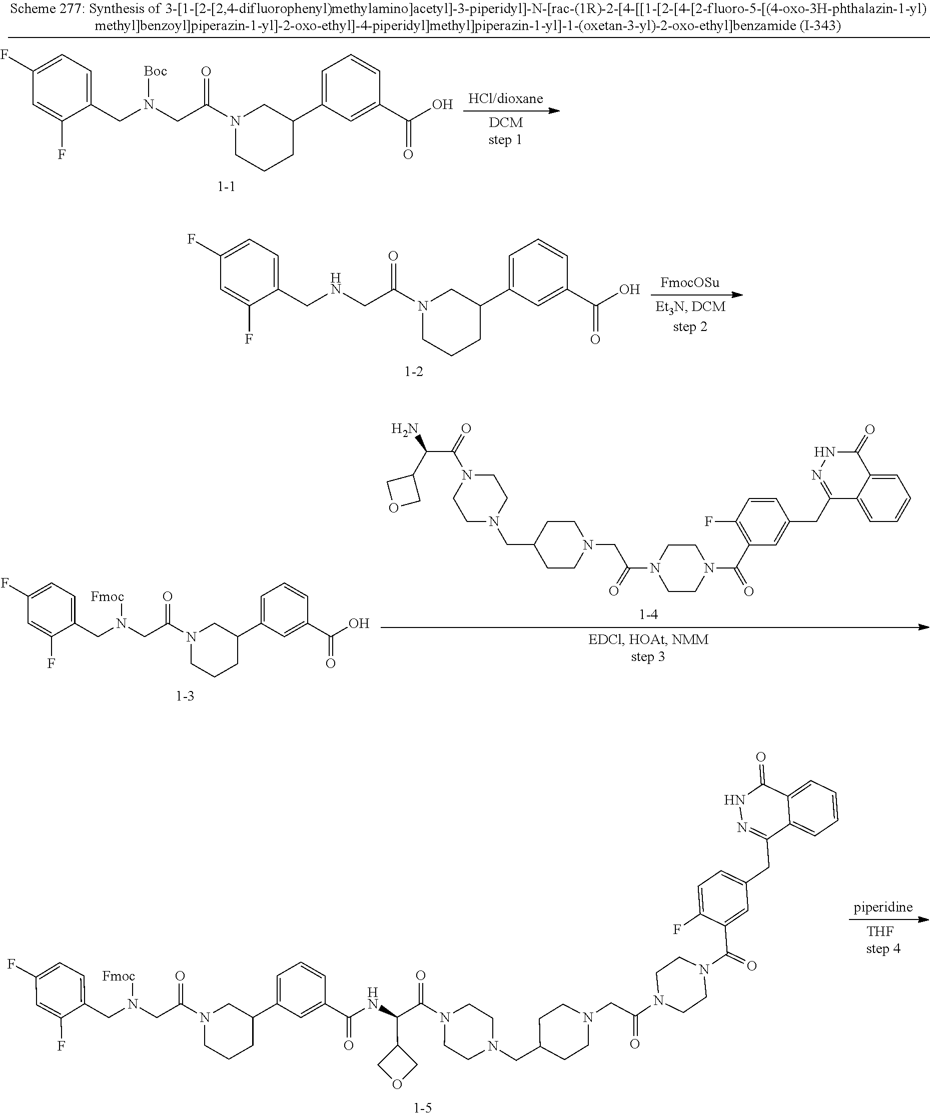

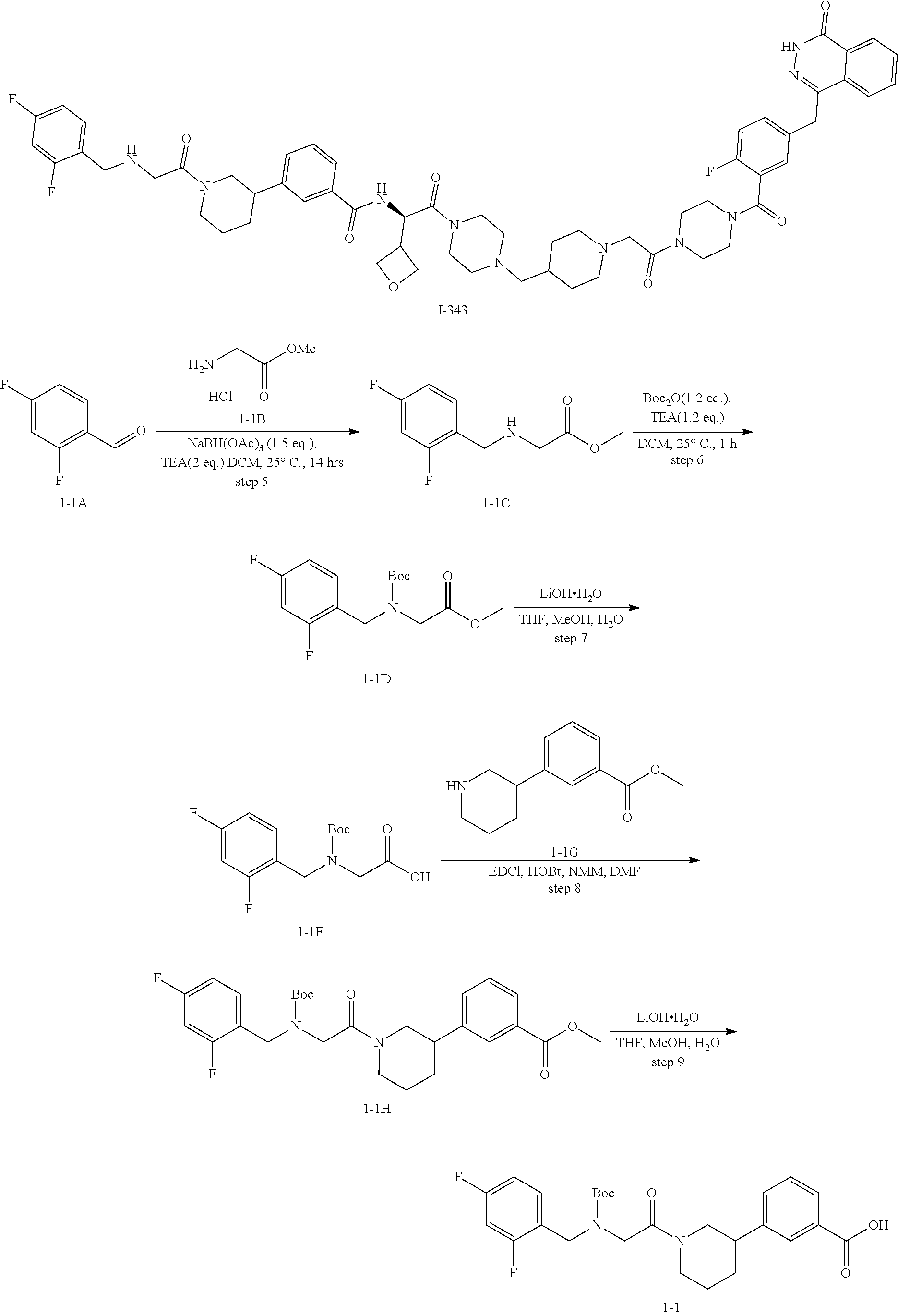

### Step 1: Synthesis of Intermediate 1-2.

To a solution of Intermediate 1-1 (500 mg, 952.41 μmol, 1 eq, HCl salt) in DCM (3 mL) was added HCl/dioxane (2 M, 6.85 mL). The mixture was stirred at 25° C. for 0.5 hr. The reaction mixture was concentrated under reduced pressure to give a residue. Without further purification, it was directly used for the next reaction. Intermediate 1-2 (300 mg, 681.54 μmol, 71.56% yield, 96.521% purity, HCl salt) was obtained as a white solid. LCMS (Method D): Retention time: 0.271 min, [M+H]⁺=389.1. ¹H NMR (400 MHz, CHLOROFORM-d) δ=7.97-7.84 (m, 2H), 7.69-7.37 (m, 3H), 7.15-7.01 (m, 2H), 4.58-4.46 (m, 1H), 4.30 (d, J=12.4 Hz, 2H), 4.25-4.04 (m, 2H), 3.77-3.65 (m, 1H), 3.29-3.25 (m, 1H), 3.25-3.11 (m, 1H), 2.92-2.66 (m, 2H), 2.08-1.97 (m, 1H), 1.92-1.76 (m, 2H), 1.74-1.52 (m, 1H).

### Step 2: Synthesis of Intermediate 1-3.

To a solution of Intermediate 1-2 (170 mg, 400.12 μmol, 1 eq, HCl salt) in DCM (2 mL) was added Et₃N (121.46 mg, 1.20 mmol, 167.08 μL, 3 eq) and Fmoc-OSu (161.97 mg, 480.15 μmol, 1.2 eq). The mixture was stirred at 25° C. for 1 hr. The reaction mixture was diluted with H₂O (2 mL) and extracted with DCM (1 mL*3). The combined organic layers were dried over Na₂SO₄, filtered and concentrated under reduced pressure to give a residue. The residue was purified by flash silica gel chromatography (ISCO; 20 g SepaFlash Silica Flash Column, Eluent of 10% MeOH/EA gradient @60 mL/min). The eluent concentrated under reduced pressure to give Intermediate 1-3 (120 mg, 196.51 μmol, 49.11% yield, 100% purity) as a white solid. LCMS (Method F): Retention time: 0.508 min, [M+H]⁺=611.1

### Step 3: Synthesis of Intermediate 1-5.

To a solution of Intermediate 1-3 (70.00 mg, 114.63 μmol, 1 eq) in DMF (1 mL) was added HOAt (31.21 mg, 229.27 μmol, 32.07 μL, 2 eq), EDCI (109.88 mg, 573.16 μmol, 5 eq) and NMM (115.95 mg, 1.15 mmol, 126.03 μL, 10 eq) and the mixture was stirred for 0.5 hr, and then was added Intermediate 1-4 (80.57 mg, 114.63 μmol, 1 eq) was added. The mixture was stirred at 25° C. for 1 hr. The reaction mixture was diluted with H₂O (3 mL) and extracted with EA (3 mL*3). The combined organic layers were washed with brine (2 mL*3), dried over Na₂SO₄, filtered and concentrated under reduced pressure to give a residue. The residue was purified by flash silica gel chromatography (ISCO; 20 g SepaFlash Silica Flash Column, Eluent of 10% EA/MeOH gradient @40 mL/min). The eluent was concentrated under reduced pressure to give Intermediate 1-5 (100 mg, 73.33 μmol, 63.97% yield, 94.9% purity) as a white solid. LCMS (Method D): Retention time: 0.466 min, [M+H]⁺=1295.6. ¹H NMR (400 MHz, CHLOROFORM-d) δ=8.36 (d, J=8.0 Hz, 1H), 7.98-7.91 (m, 1H), 7.90-7.79 (m, 3H), 7.79-7.71 (m, 3H), 7.63-7.55 (m, 1H), 7.54-7.43 (m, 4H), 7.42-7.34 (m, 3H), 7.32-7.23 (m, 2H), 7.11 (s, 2H), 6.94-6.75 (m, 2H), 4.59 (s, 9H), 4.45-4.35 (m, 5H), 4.26-4.17 (m, 2H), 4.11-4.03 (m, 1H), 3.87-3.71 (m, 6H), 3.68-3.59 (m, 5H), 3.54-3.45 (m, 5H), 2.52-2.31 (m, 6H), 2.24-2.16 (m, 2H), 1.87-1.78 (m, 4H), 1.47 (s, 1H), 1.39-1.28 (m, 7H). ¹⁹F NMR (376 MHz, CHLOROFORM-d) δ=−120.67.

### Step 4: Synthesis of I-343

A solution of Intermediate 1-5 (90 mg, 69.47 μmol, 1 eq) in THF (1 mL) and piperidine (0.12 mL) was stirred at 25° C. for 1 hr. The reaction mixture was diluted with H₂O (1 mL) and extracted with EA (2 mL*3). The combined organic layers were dried over Na₂SO₄, filtered and concentrated under reduced pressure to give a residue. The residue was purified by prep-HPLC (FA condition). The eluent was concentrated and lyophilized to give I-343 (7.72 mg, 7.19 μmol, 10.35% yield, 99.9% purity) as a white solid. LCMS (Method F): Retention time: 0.586 min, [M+H]⁺=1073.8. SFC: Retention time: 3.336, 3.561 min. ¹H NMR (400 MHz, METHANOL-d4) δ=¹H NMR (400 MHz, METHANOL-d4) δ=8.37 (d, J=7.2 Hz, 1H), 7.98-7.92 (m, 1H), 7.90-7.80 (m, 2H), 7.78 (s, 1H), 7.76-7.70 (m, 1H), 7.52-7.42 (m, 4H), 7.37 (s, 1H), 7.18-7.13 (m, 1H), 6.99-6.88 (m, 2H), 5.49-5.46 (m, 1H), 4.80 (d, J=7.2 Hz, 3H), 4.68-4.65 (m, 1H), 4.54 (s, 1H), 4.45-4.34 (m, 3H), 3.90-3.78 (m, 4H), 3.74-3.70 (m, 2H), 3.68-3.64 (m, 2H), 3.63-3.57 (m, 2H), 3.54 (s, 2H), 3.52-3.47 (m, 2H), 3.28-3.21 (m, 2H), 3.18-3.05 (m, 1H), 2.93-2.87 (m, 2H), 2.84-2.64 (m, 2H), 2.49-2.31 (m, 4H), 2.24-1.97 (m, 6H), 1.88-1.82 (m, 2H), 1.81-1.66 (m, 3H), 1.65-1.47 (m, 2H), 1.38-1.07 (m, 3H). ¹⁹F NMR (376 MHz, METHANOL-d4) δ=−120.77.

### Step 5: Synthesis of Intermediate 1-1C.

To a solution of Intermediate 1-1A (10 g, 70.37 mmol, 7.66 mL, 1 eq) and Intermediate 1-1B (22.80 g, 140.74 mmol, 2 eq, HCl salt) in DCM (100 mL) was added TEA (14.24 g, 140.74 mmol, 19.59 mL, 2 eq). The mixture was stirred at 25° C. for 2 hr. Then the NaBH(OAc)₃ (22.37 g, 105.56 mmol, 1.5 eq) was added and the mixture was stirred at 25° C. for 12 hr. The reaction mixture was diluted with water (80 mL) and extracted with EA (30 mL*3). The combined organic layers were dried over Na₂SO₄, filtered and concentrated under reduced pressure to give a residue. The residue was used in the next step without further purification. Intermediate 1-1C (15 g, 69.70 mmol, 99.05% yield) was obtained as a yellow solid. LCMS (Method D): Retention time: 0.123 min, [M+H]⁺=216.2.

### Step 6: Synthesis of Intermediate 1-1D.

To a solution of Intermediate 1-1C (15 g, 69.70 mmol, 1 eq) in DCM (150 mL) was added TEA (8.46 g, 83.64 mmol, 11.64 mL, 1.2 eq) and Boc₂O (18.26 g, 83.64 mmol, 19.22 mL, 1.2 eq). The mixture was stirred at 25° C. for 1 hr. The reaction mixture was diluted with water (100 mL) and extracted with EA (50 mL*3). The combined organic layers were dried over Na₂SO₄, filtered and concentrated under reduced pressure to give a residue. The residue was purified by flash silica gel chromatography (ISCO; 220 g SepaFlash Silica Flash Column, Eluent of 0˜100% Petroleum ether/Ethyl acetate @60 mL/min). The eluent was concentrated under reduced pressure to give Intermediate 1-1D (10.6 g, 33.62 mmol, 48.23% yield) as a yellow solid. LCMS (Method D): Retention time: 0.400 min, [M-Boc+H]⁺=216.1.

### Step 7: Synthesis of Intermediate 1-1F.

To a solution of Intermediate 1-1D (10.5 g, 33.30 mmol, 1 eq) in MeOH (35 mL), THF (35 mL), and H₂O (35 mL) was added LiOH·H₂O (2.79 g, 66.60 mmol, 2 eq). The mixture was stirred at 25° C. for 1 hr. The mixture was adjusted to pH=5 with citric acid and extracted with EA (20 mL*3). The combined organic layers were dried over Na₂SO₄, filtered and concentrated under reduced pressure to give a residue. The residue was used in the next step without further purification. Intermediate 1-1F (9.7 g, 32.20 mmol, 96.68% yield) was obtained as a colorless oil. LCMS (Method D): Retention time: 0.409 min, [M+Na]⁺=324.0.

### Step 8: Synthesis of Intermediate 1-1H.

To a solution of Intermediate 1-1F (7.5 g, 24.89 mmol, 1 eq) in DMF (75 mL) was added EDCI (23.86 g, 124.47 mmol, 5 eq), HOAt (6.78 g, 49.79 mmol, 6.96 mL, 2 eq) and NMM (25.18 g, 248.93 mmol, 27.37 mL, 10 eq), then the Intermediate 1-1G (5.46 g, 24.89 mmol, 1 eq) was added. The mixture was stirred at 25° C. for 0.5 hr. The reaction mixture was diluted with water (60 mL) and extracted with EA (30 mL*3). The combined organic layers were dried over Na₂SO₄, filtered and concentrated under reduced pressure to give a residue. The residue was purified by flash silica gel chromatography (ISCO; 80 g SepaFlash Silica Flash Column, Eluent of 0˜100% Petroleum ether/Ethyl acetate @70 mL/min). The eluent was concentrated under reduced pressure to give Intermediate 1-1H (9 g, 17.91 mmol, 71.94% yield) as yellow oil. LCMS (Method D): Retention time: 0.521 min, [M+H]⁺=503.2. ¹H NMR (400 MHz, METHANOL-d₄) δ=8.00-7.86 (m, 2H), 7.54 (d, J=7.6 Hz, 1H), 7.48-7.33 (m, 2H), 6.99-6.89 (m, 2H), 4.60-4.44 (m, 3H), 3.91 (s, 4H), 3.69 (t, J=4.4 Hz, 1H), 3.21-3.07 (m, 1H), 3.00-2.85 (m, 1H), 2.83-2.66 (m, 2H), 2.45-2.27 (m, 1H), 1.85 (t, J=12.8 Hz, 2H), 1.62 (s, 1H), 1.44 (d, J=3.6 Hz, 9H).

### Step 9: Synthesis of Intermediate 1-1.

To a solution of Intermediate 1-1H (9 g, 17.91 mmol, 1 eq) in THF (30 mL), MeOH (30 mL), and H₂O (30 mL) was added LiOH·H₂O (1.50 g, 35.82 mmol, 2 eq). The mixture was stirred at 25° C. for 2 hr. The mixture was adjusted to pH=5 with citric acid and extracted with EA (30 mL*3). The combined organic layers were dried over Na₂SO₄, filtered and concentrated under reduced pressure to give a residue. The residue was used in the next step without further purification. Intermediate 1-1 (10 g, crude) was obtained as a yellow solid. LCMS (Method D): Retention time: 0.470 min, [M+Na]⁺=511.4. ¹H NMR (400 MHz, METHANOL-d₄) δ=8.03-7.86 (m, 2H), 7.54 (d, J=7.6 Hz, 1H), 7.47-7.35 (m, 2H), 7.00-6.89 (m, 2H), 4.59-4.43 (m, 3H), 4.28-4.16 (m, 1H), 3.89 (d, J=17.6 Hz, 1H), 3.25-3.08 (m, 1H), 2.88-2.69 (m, 2H), 2.08-2.03 (m, 1H), 2.00 (s, 1H), 1.92-1.78 (m, 2H), 1.72-1.56 (m, 1H), 1.48-1.42 (m, 9H). ¹⁹F NMR (377 MHz, METHANOL-d₄) δ=−113.423, δ=−115.472, −115.862.

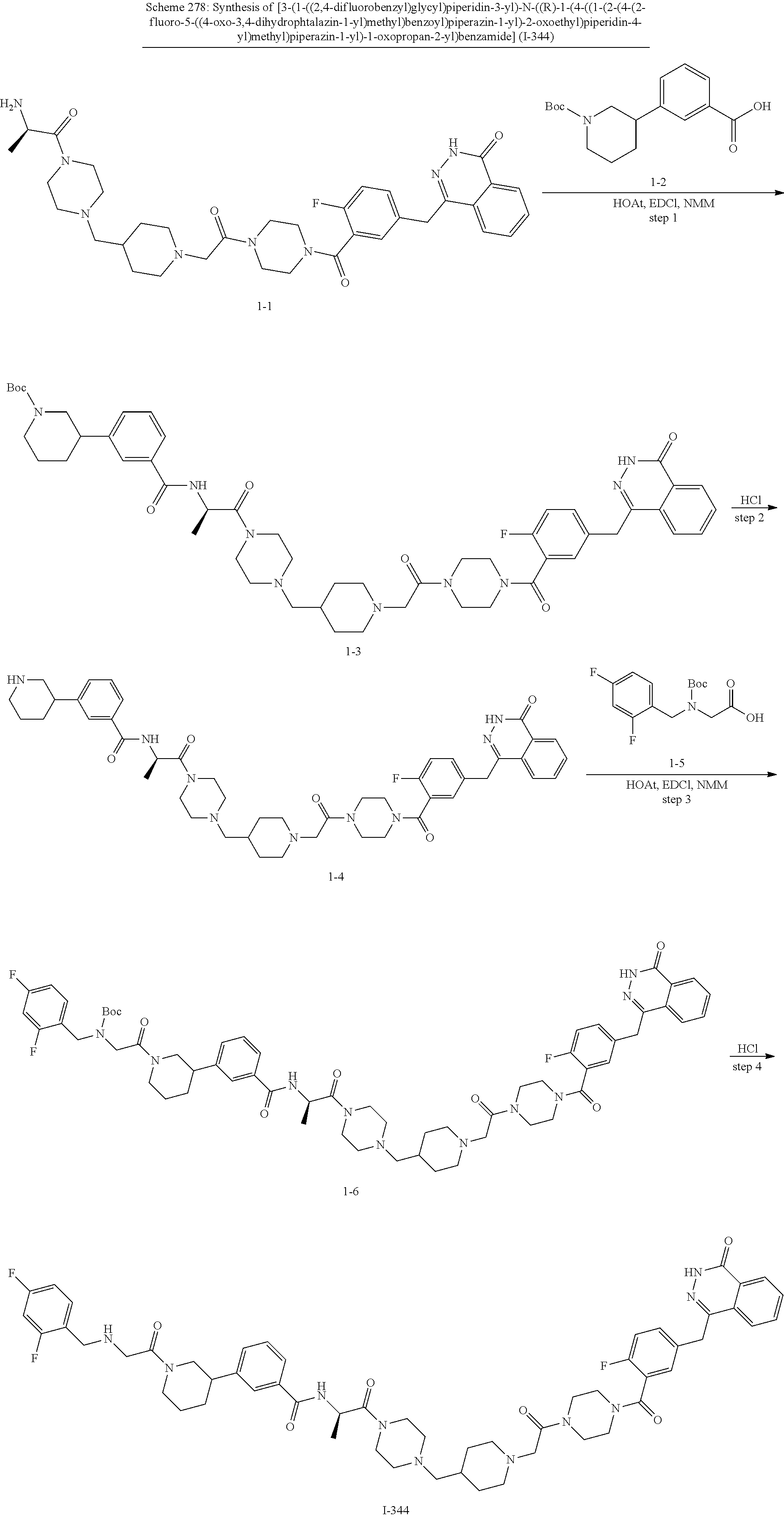

### Step 1: Synthesis of Intermediate 1-3

To a solution of Intermediate 1-1 (80 mg, 114.74 μmol, 1 eq, HCl salt) and Intermediate 1-2 (42.04 mg, 137.69 μmol, 1.2 eq) in DMF (1 mL) was added NMM (58.03 mg, 573.69 μmol, 63.07 μL, 5 eq), EDCI (65.99 mg, 344.21 μmol, 3 eq), HOAt (15.62 mg, 114.74 gmol, 16.05 μL, 1 eq). The mixture was stirred at 25° C. for 0.5 hr. The reaction mixture was washed with H₂O (2 mL) at 25° C., and the mixture was extracted with EA (2 ml*2), then the combined organic phase was dried with anhydrous sodium sulfate, filtered and the filtrate was concentrated to give the crude product. The crude product was purified by prep-HPLC (0.1% FA), the eluent was concentrated to remove ACN and lyophilized to give Intermediate 1-3 (50 mg, 52.74 μmol, 45.96% yield) as a white solid. LCMS (Method D): Retention time: 0.371 min (M+H)⁺=948.5.

### Step 2: Synthesis of Intermediate 1-4

A solution of Intermediate 1-3 (40 mg, 42.19 μmol, 1 eq) in HCl/dioxane (2M, 0.1 mL) and DCM (0.2 mL) was stirred at 25° C. for 0.5 hr. The reaction mixture was concentrated under reduced pressure to give Intermediate 1-4 (40 mg, crude, HCl salt) as a white solid. LCMS (Method D): Retention time: 0.264 min, (M+H)⁺=848.4.

### Step 3: Synthesis of Intermediate 1-6

To a solution of Intermediate 1-4 (40 mg, 45.22 μmol, 1 eq, HCl salt) and Intermediate 1-5 (16.35 mg, 54.27 μmol, 1.2 eq) in DMF (1 mL) was added NMM (22.87 mg, 226.12 μmol, 24.86 μL, 5 eq), EDCI (26.01 mg, 135.67 μmol, 3 eq), and HOAt (6.16 mg, 45.22 μmol, 6.33 μL, 1 eq). The mixture was stirred at 25° C. for 0.5 hr. The reaction mixture was diluted with H₂O (2 mL) and extracted with EA (3 mL*2). The combined organic layers were washed with NaCl (aq) (2 mL*2), dried over Na₂SO₄, filtered and concentrated under reduced pressure to give a residue. The residue was purified by prep-HPLC (0.1% FA) the eluent was concentrated to remove ACN and lyophilized to give Intermediate 1-6 (45 mg, 39.78 μmol, 87.96% yield) as a white solid. LCMS (Method D): Retention time: 0.420 min, (M+H)⁺=1131.5.

### Step 4: Synthesis of I-344

A solution of Intermediate 1-6 (20 mg, 17.68 μmol, 1 eq) in HCl/dioxane (2M, 0.1 mL) was stirred at 25° C. for 0.5 hr. The reaction mixture was concentrated under reduced pressure to give a residue. The residue was purified by prep-HPLC (column: CD02-Waters Xbidge BEH C18 150*25*10 um; mobile phase: [water(NH₃H2O)-ACN]; gradient: 28%-58% B over 10 min) the eluent was concentrated to remove ACN and lyophilized to give I-344 (8.27 mg, 6.96 μmol, 39.35% yield, 96.3% purity, TFA salt) as a white solid. LCMS (Method D): Retention time: 0.314 min, (M+H)⁺=1031.5. ¹H NMR (400 MHz, METHANOL-d4) δ=8.37 (d, J=7.2 Hz, 1H), 8.01-7.92 (m, 1H), 7.91-7.81 (m, 2H), 7.78 (s, 1H), 7.75-7.68 (m, 1H), 7.52-7.42 (m, 4H), 7.40-7.34 (m, 1H), 7.19-7.15 (m, 1H), 7.02-6.90 (m, 2H), 5.08-5.02 (m, 1H), 4.58 (s, 2H), 4.39 (s, 2H), 3.86 (d, J=10.8 Hz, 2H), 3.82-3.78 (m, 1H), 3.75 (s, 1H), 3.72-3.65 (m, 4H), 3.61-3.57 (m, 2H), 3.53 (s, 3H), 3.19-3.10 (m, 2H), 3.08-2.99 (m, 2H), 2.85-2.76 (m, 1H), 2.76-2.69 (m, 1H), 2.56-2.37 (m, 5H), 2.35-2.14 (m, 5H), 2.09-2.02 (m, 1H), 1.94-1.75 (m, 5H), 1.66-1.56 (m, 2H), 1.45-1.39 (m, 3H), 1.34-1.20 (m, 3H).

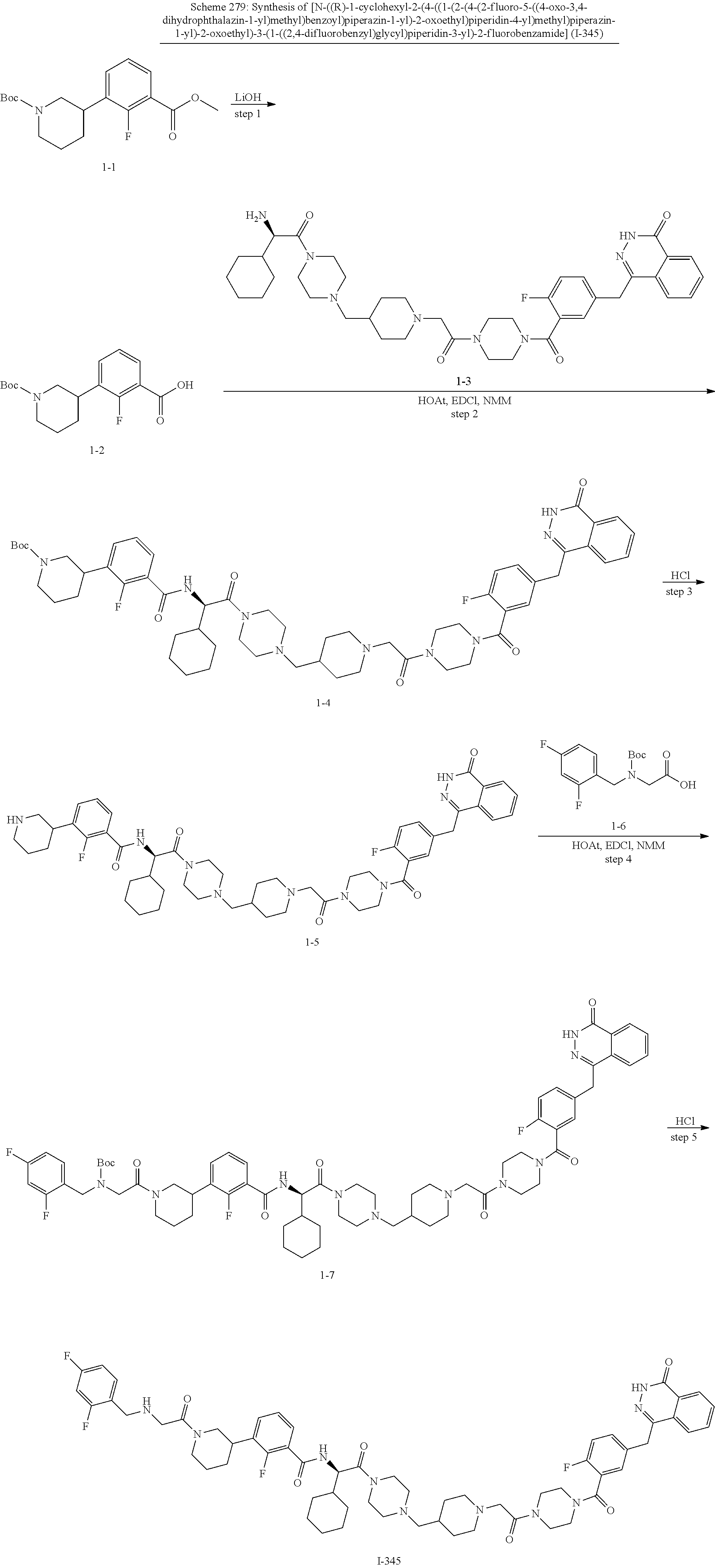

### Step 1: Synthesis of Intermediate 1-2

To a solution of Intermediate 1-1 (400 mg, 1.19 mmol, 1 eq) in THF (4 mL), MeOH (4 mL) and H₂O (4 mL) was added LiOH·H₂O (149.25 mg, 3.56 mmol, 3 eq). The mixture was stirred at 25° C. for 0.5 hr. The reaction mixture was adjusted to pH=6 by aq. HCl (2M) and concentrated to remove ACN and lyophilized to give Intermediate 1-2 (360 mg, crude) as a white solid. LCMS (Method D): Retention time: 0.428 min, (M+H)⁺=346.2.

### Step 2: Synthesis of Intermediate 1-4

To a solution of Intermediate 1-2 (84.62 mg, 235.18 μmol, 1.2 eq, HCl salt) and Intermediate 1-3 (150 mg, 195.99 μmol, 1 eq, HCl salt) in DMF (1.5 mL) was added EDCI (112.71 mg, 587.96 μmol, 3 eq), NMM (99.12 mg, 979.93 μmol, 107.74 μL, 5 eq), HOAt (26.68 mg, 195.99 μmol, 27.42 μL, 1 eq). The mixture was stirred at 25° C. for 1 hr. The reaction mixture was diluted with H₂O (6 mL) and extracted with EA (5 mL*2). The combined organic layers were washed with NaCl (aq) (5 mL*2), dried over Na₂SO₄, filtered and concentrated under reduced pressure to give a residue. The residue was purified by prep-HPLC (0.1% FA) and the eluent was concentrated to remove ACN and lyophilized to give Intermediate 1-4 (110 mg, 106.36 μmol, 54.27% yield) as a white solid. SFC showed two peaks (peak1: 1.673 min, 46.79%, peak 2: 1.934 min, 52.21%). LCMS (Method D): Retention time: 0.418 min, (M+H)⁺=1034.6.

### Step 3: Synthesis of Intermediate 1-5

A solution of Intermediate 1-4 (0.1 g, 96.69 μmol, 1 eq) in HCl/dioxane (2M, 1 mL) and DCM (2 mL) was stirred at 25° C. for 0.5 hr. The reaction mixture was concentrated under reduced pressure to give Intermediate 1-5 (100 mg, crude, HCl salt) as a white solid. LCMS (Method D): Retention time: 0.300 min, (M+H)=934.5.

### Step 4: Synthesis of Intermediate 1-7

To a solution of Intermediate 1-5 (100 mg, 103.03 μmol, 1 eq, HCl salt) and Intermediate 1-6 (31.04 mg, 103.03 μmol, 1 eq) in DMF (1 mL) was added NMM (52.11 mg, 515.15 μmol, 56.64 μL, 5 eq) and EDCI (59.25 mg, 309.09 μmol, 3 eq), HOAt (14.02 mg, 103.03 μmol, 14.41 μL, 1 eq). The mixture was stirred at 25° C. for 0.5 hr. The reaction mixture was washed with H₂O (2 mL) at 25° C., and mixture the was extracted with EA (3 ml*2). The combined organic phase was dried with anhydrous sodium sulfate, filtered and the filtrate was concentrated to give the crude product. The crude product was purified by prep-HPLC (0.1% FA), the eluent was concentrated to remove ACN and lyophilized to give Intermediate 1-7 (80 mg, 65.71 μmol, 63.78% yield) as a white solid. SFC showed two peaks (peak 1: 2.042 min, 46.375%; peak 2: 2.359 min, 53.62%). LCMS (Method D): Retention time: 0.437 min, (M+H)⁺=1217.7.

### Step 5: Synthesis of I-345

A solution of Intermediate 1-7 (60 mg, 49.29 μmol, 1 eq) in HCl/dioxane (2M, 0.5 mL) and DCM (1 mL) was stirred at 25° C. for 0.5 hr. The reaction mixture was concentrated under reduced pressure to give a residue. The residue was purified by prep-HPLC (0.1% FA), the eluent was concentrated to remove ACN and lyophilized to give I-345 (54.42 mg, 45.30 μmol, 91.92% yield, 96.8% purity, FA salt) as a white solid. LCMS (Method D): Retention time: 0.326 min, (M+H)⁺=1117.6. ¹H NMR (400 MHz, METHANOL-d4) δ=8.59-8.48 (m, 1H), 8.37 (d, J=7.6 Hz, 1H), 8.01-7.93 (m, 1H), 7.91-7.80 (m, 2H), 7.67-7.60 (m, 1H), 7.58-7.48 (m, 3H), 7.39-7.36 (m, 1H), 7.33-7.24 (m, 1H), 7.21-7.07 (m, 3H), 4.62-4.55 (m, 2H), 4.39 (s, 2H), 4.36-4.32 (m, 3H), 4.30-4.26 (m, 1H), 4.23-4.13 (m, 2H), 3.86-3.76 (m, 4H), 3.75-3.68 (m, 4H), 3.61-3.50 (m, 3H), 3.43 (d, J=11.2 Hz, 2H), 3.37 (d, J=13.2 Hz, 3H), 3.25-3.15 (m, 6H), 3.14-3.04 (m, 2H), 2.95-2.74 (m, 2H), 2.44-2.30 (m, 1H), 2.22 (d, J=12.0 Hz, 2H), 2.07-2.01 (m, 1H), 1.97-1.86 (m, 4H), 1.83-1.67 (m, 7H), 1.34-1.24 (m, 3H), 1.22-1.13 (m, 2H).

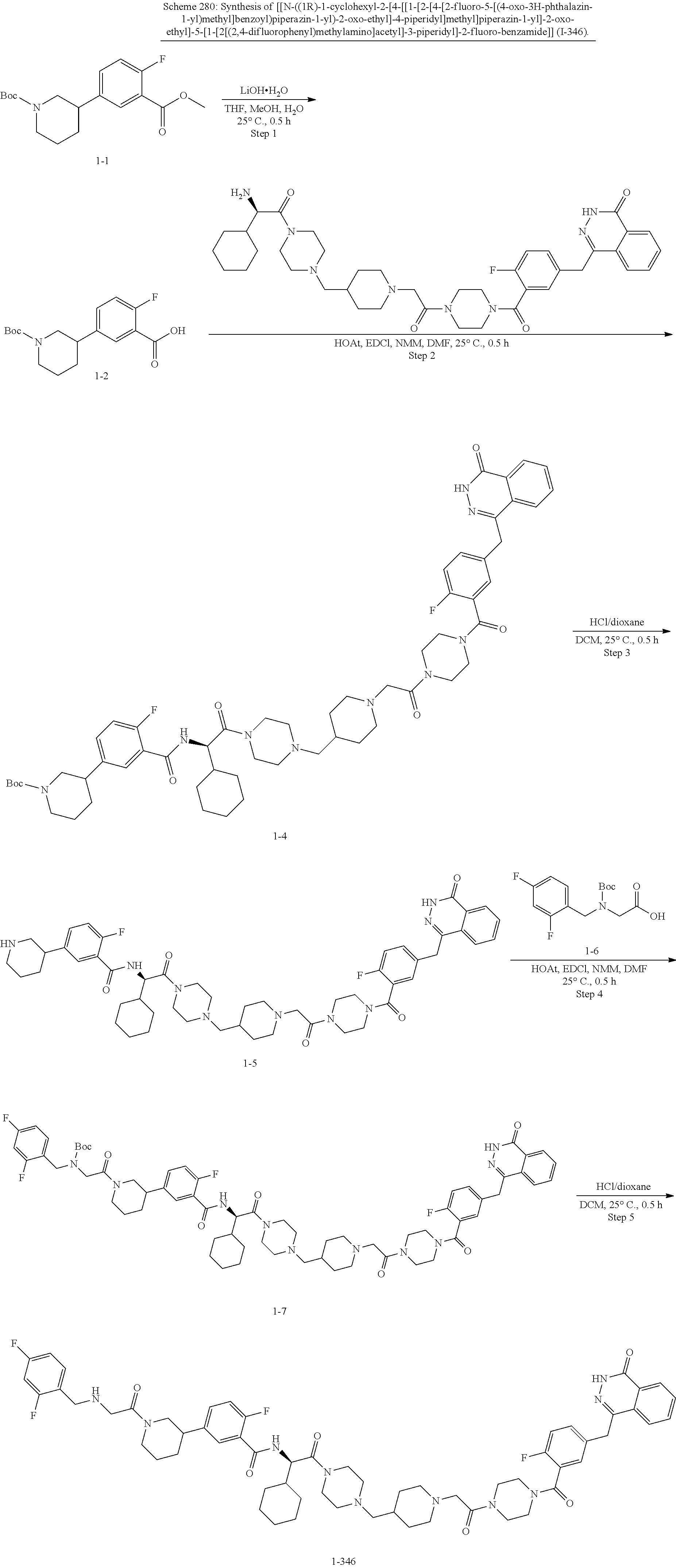

### Step 1: Synthesis of Intermediate 1-2

To a solution of intermediate 1-1 (300 mg, 889.19 μmol, 1 eq) in MeOH (1 mL), THF (1 mL) and H₂O (1 mL) was added LiOH·H₂O (111.94 mg, 2.67 mmol, 3 eq). The mixture was stirred at 25° C. for 0.5 hr. The reaction mixture was poured into H₂O (10 mL) at 25° C., the pH was adjusted to 3 with 1M aqueous HCl, and then extracted with DCM (10 mL*3). The combined organic layers were dried over Na₂SO₄, filtered and concentrated under reduced pressure to give Intermediate 1-2 (270 mg, crude) as a white solid. LCMS (Method D): Rt=0.441 min, (M+H)⁺=224.2. ¹H NMR (400 MHz, CHLOROFORM-d) δ=7.83-7.80 (m, 1H), 7.39-7.35 (m, 1H), 7.11-6.99 (m, 1H), 4.25-3.96 (m, 2H), 2.77-2.58 (m, 3H), 1.96 (d, J=9.6 Hz, 1H), 1.77-1.65 (m, 1H), 1.62-1.50 (m, 2H), 1.41 (s, 9H).

### Step 2: Synthesis of Intermediate 1-4

To a solution of intermediate 1-2 (150 mg, 195.99 μmol, 1 eq, HCl salt) and intermediate 1-3 (69.71 mg, 215.58 μmol, 1.1 eq) in DMF (1.5 mL) was added EDCI (112.71 mg, 587.96 μmol, 3 eq), NMM (99.12 mg, 979.93 μmol, 107.73 μL, 5 eq) and HOAt (26.67 mg, 195.99 μmol, 27.42 μL, 1 eq). The mixture was stirred at 25° C. for 0.5 hr. The reaction mixture was poured into H₂O (2 mL) and extracted with EA (2 mL*3). The combined organic layers were dried over Na₂SO₄, filtered and concentrated under reduced pressure to give a residue. The residue was purified by flash silica gel chromatography (ISCO; 4 g SepaFlash Silica Flash Column, Eluent of 0˜100% DCM/MeOH@18 mL/min, DCM/MeOH=10:1, Rf=0.2) and the eluent was concentrated under reduced pressure to give Intermediate 1-4 (50 mg, crude) as a yellow gum. LCMS (Method D): Rt=0.394 min, (M+H)⁺=1034.5.

### Step 3: Synthesis of Intermediate 1-5

To a solution of intermediate 1-4 (144 mg, 139.23 μmol, 1 eq) in DCM (1 mL) was added HCl/dioxane (2 M, 69.62 μL, 1 eq) and was stirred at 25° C. for 0.5 hr. The reaction mixture was concentrated under reduced pressure to give Intermediate 1-5 (160 mg, crude, HCl salt) as a yellow solid. LCMS (Method D): Rt=0.287 min, (M+H)⁺=934.4.

### Step 4: Synthesis of Intermediate 1-7

To a solution of intermediate 1-5 (160 mg, 164.85 μmol, 1 eq, HCl salt) and intermediate 1-6 (59.60 mg, 197.82 μmol, 1.2 eq) in DMF (1.6 mL) was added EDCI (94.81 mg, 494.55 μmol, 3 eq), NMM (83.37 mg, 824.25 μmol, 90.62 μL, 5 eq) and HOAt (22.44 mg, 164.85 μmol, 23.06 μL, 1 eq). The mixture was stirred at 25° C. for 0.5 hr. The reaction mixture was poured into H₂O (5 mL) and extracted with EA (5 mL*3). The combined organic layers were dried over Na₂SO₄, filtered and concentrated under reduced pressure to give a residue. The residue was purified by flash silica gel chromatography (ISCO; 12 g SepaFlash Silica Flash Column, Eluent of 0˜100% DCM/MeOH@36 mL/min, DCM/MeOH=10:1, Rf=0.3) and the eluent was concentrated under reduced pressure to give Intermediate 1-7 (122 mg, crude) as a yellow gum. LCMS (Method D): Rt=0.416 min, (M+H)⁺=1218.1.

### Step 5: Synthesis of I-346

A solution of intermediate 1-7 (122 mg, 100.21 μmol, 1 eq) in DCM (1 mL) was added HCl/dioxane (2 M, 0.2 mL, 3.99 eq) and it was stirred at 25° C. for 0.5 hr. The reaction mixture was concentrated under reduced pressure to give a residue. The residue was purified by prep-HPLC (column: Phenomenex luna C18 150*25 mm*10 um; mobile phase: [water (HCl)-ACN]; gradient: 15%-45% B over 10 min) and concentrated under reduced pressure to remove ACN, and lyophilized to give I-346 (32.84 mg, 29.33 μmol, 29.26% yield, 99.77% purity) as a yellow solid. LCMS (Method D): Rt=0.304 min, (M+H)⁺=1117.5. ¹H NMR (400 MHz, METHANOL-d4) δ=8.37 (d, J=7.6 Hz, 1H), 8.00-7.93 (m, 1H), 7.92-7.80 (m, 2H), 7.72-7.58 (m, 2H), 7.51 (s, 2H), 7.43-7.34 (m, 1H), 7.24-7.06 (m, 4H), 4.89 (d, J=2.0 Hz, 2H), 4.80-4.67 (m, 1H), 4.61-4.50 (m, 2H), 4.39 (s, 2H), 4.37-4.26 (m, 4H), 4.25-4.04 (m, 2H), 3.88-3.81 (m, 2H), 3.80-3.69 (m, 6H), 3.60-3.52 (m, 2H), 3.44-3.33 (m, 5H), 3.28-3.15 (m, 5H), 2.92-2.73 (m, 2H), 2.45-2.32 (m, 1H), 2.29-2.13 (m, 2H), 2.05 (d, J=12.4 Hz, 2H), 1.94-1.86 (m, 3H), 1.85-1.66 (m, 8H), 1.39-1.24 (m, 3H), 1.20-1.11 (m, 2H). ¹⁹F NMR (376 MHz, CHLOROFORM-d) δ=−108.395, δ=−112.831, δ=−118.235, δ=−120.757.

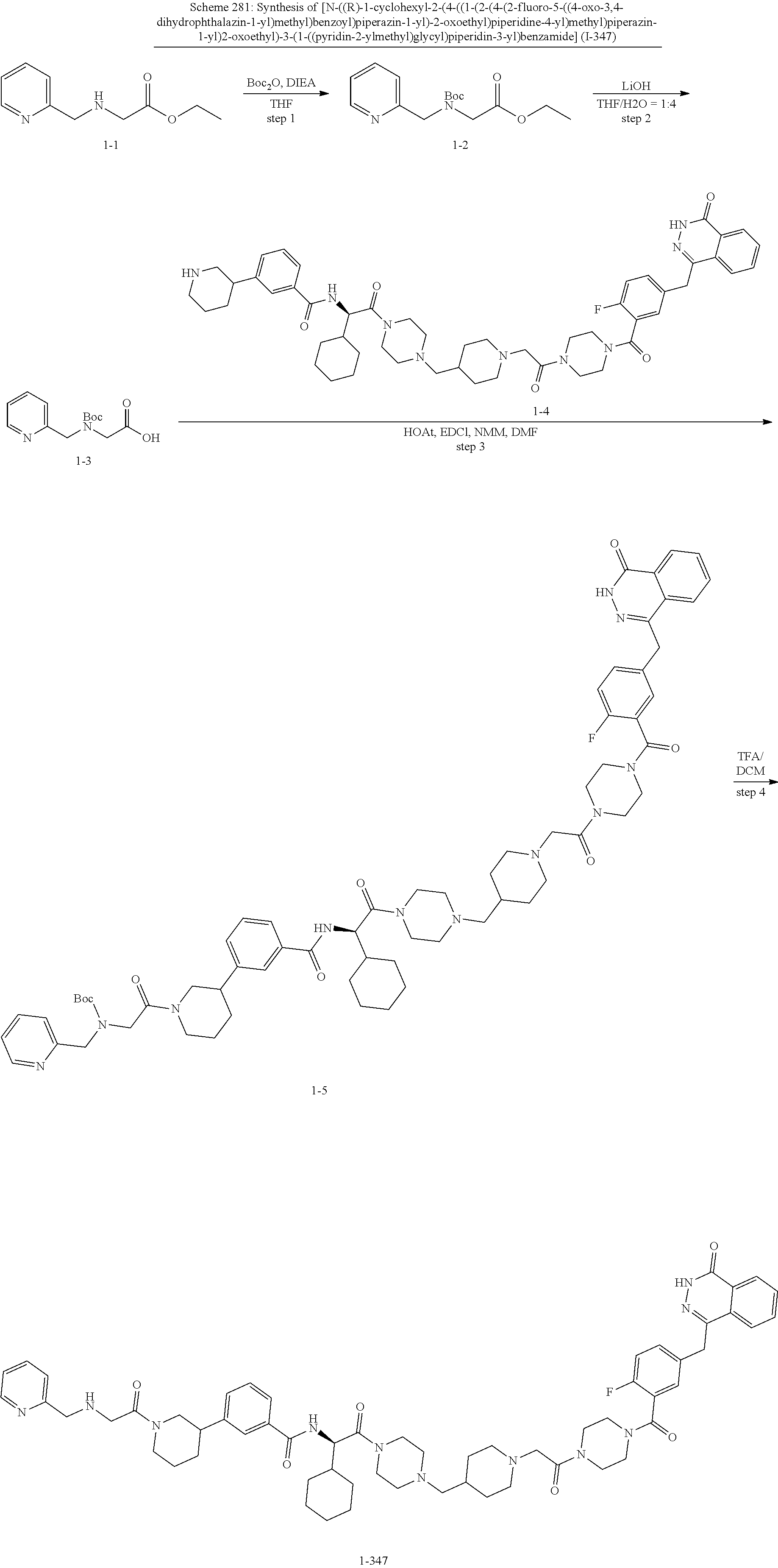

### Step 1: Synthesis of Intermediate 1-2

To a solution of Intermediate 1-1 (500 mg, 2.57 mmol, 1 eq), Boc₂O (674.19 mg, 3.09 mmol, 709.67 μL, 1.2 eq) in DCM (5 mL) was added DIEA (665.41 mg, 5.15 mmol, 896.78 μL, 2 eq). The mixture was stirred at 25° C. for 0.5 hr. The reaction mixture was concentrated under reduced pressure to give a residue. The residue was purified by flash silica gel chromatography (ISCO; 20 g SepaFlash Silica Flash Column, Eluent of 0˜20% Ethyl acetate/Petroleum ethergradient @60 mL/min) and the eluent was concentrated under reduced pressure to give Intermediate 1-2 (600 mg, 2.04 mmol, 79.18% yield) as a colorless oil. LCMS (Method D): Retention time: 0.314 min, (M+H)⁺=295.1.

### Step 2: Synthesis of Intermediate 1-3

To a solution of Intermediate 1-2 (600 mg, 2.04 mmol, 1 eq) in THF (6 mL), H₂O (6 mL) and MeOH (6 mL) was added LiOH·H₂O (256.62 mg, 6.12 mmol, 3 eq). The mixture was stirred at 25° C. for 0.5 hr. The reaction mixture was was concentrated to remove THF/MeOH and lyophilized to give Intermediate 1-3 (500 mg, crude) as a colorless oil. LCMS (Method D): Retention time: 0.214 min, (2*M+Na)⁺=555.4.

### Step 3: Synthesis of Intermediate 1-5

To a solution of Intermediate 1-3 (150 mg, 163.73 μmol, 1 eq) and Intermediate 1-4 (59.48 mg, 196.48 μmol, 1.2 eq, HCl salt) in DMF (1.5 mL) was added EDCI (94.16 mg, 491.19 μmol, 3 eq) and NMM (82.80 mg, 818.66 μmol, 90.01 μL, 5 eq), HOAt (22.29 mg, 163.73 μmol, 22.90 μL, 1 eq). The mixture was stirred at 25° C. for 0.5 hr. The reaction mixture was diluted with H₂O (2 mL) and extracted with EA (3 mL*2). The combined organic layers were washed with NaCl (aq) (1 mL*2), dried over Na₂SO₄, filtered and concentrated under reduced pressure to give a residue. The residue was purified by prep-HPLC (0.1% FA) and the eluent was concentrated to remove ACN and lyophilized to give Intermediate 1-5 (110 mg, 94.47 μmol, 57.7% yield) as a white solid. LCMS (Method D): Retention time: 0.366 min, (M+H)⁺=1164.8.

### Step 4: Synthesis of I-347

To a solution of Intermediate 1-5 (30 mg, 25.76 μmol, 1 eq) in DCM (0.3 mL) was added TFA (92.10 mg, 807.73 μmol, 60.00 μL, 31.35 eq). The mixture was stirred at 25° C. for 2 hr. The reaction mixture was concentrated under reduced pressure to give a residue. The residue was purified by prep-HPLC (0.1% TFA) and the eluent was concentrated to remove ACN and lyophilized to give I-347 (7.28 mg, 5.70 μmol, 22.14% yield, 92.3% purity, TFA salt) as a white solid. LCMS (Method D): Retention time: 0.304 min, (M+H)⁺=1064.8. ¹H NMR (400 MHz, METHANOL-d4) δ=8.64 (d, J=4.4 Hz, 1H), 8.58-8.48 (m, 1H), 8.37 (d, J=7.6 Hz, 1H), 7.99-7.92 (m, 1H), 7.92-7.81 (m, 3H), 7.80-7.76 (m, 1H), 7.73 (d, J=7.6 Hz, 1H), 7.56-7.41 (m, 5H), 7.40-7.34 (m, 1H), 7.19-7.15 (m, 1H), 4.60 (d, J=14.0 Hz, 1H), 4.57-4.48 (m, 1H), 4.45 (s, 1H), 4.42-4.37 (m, 3H), 4.32-4.22 (m, 4H), 4.21-4.13 (m, 1H), 4.05-3.92 (m, 1H), 3.85-3.81 (m, 1H), 3.80-3.74 (m, 2H), 3.73-3.64 (m, 3H), 3.57-3.51 (m, 2H), 3.36 (d, J=8.4 Hz, 4H), 3.28-3.19 (m, 2H), 3.18-2.96 (m, 4H), 2.93-2.84 (m, 1H), 2.83-2.73 (m, 1H), 2.42-2.25 (m, 1H), 2.23-2.00 (m, 4H), 1.99-1.85 (m, 4H), 1.83-1.41 (m, 8H), 1.35-1.21 (m, 4H), 1.19-1.07 (m, 2H).

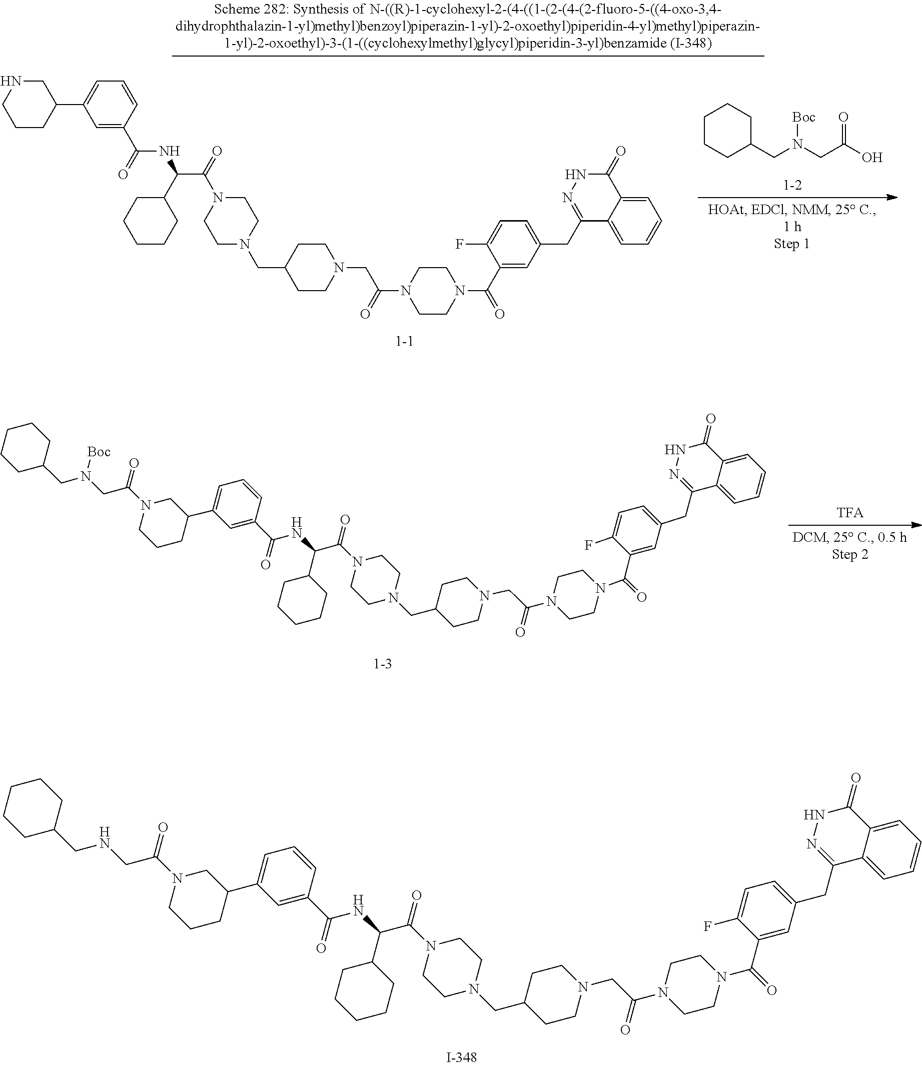 [SMILES: O=C(N[C@@H](C(=O)N1CCN(CC2CCN(CC(=O)N3CCN(C(=O)c4cc(Cc5n[nH]c(=O)c6ccccc56)ccc4F)CC3)CC2)CC1)C1CCCCC1)c1cccc(C2CCCNC2)c1 ; CC(C)(C)OC(=O)N(CC(=O)O)CC1CCCCC1 ; CC(C)(C)OC(=O)N(CC(=O)N1CCCC(c2cccc(C(=O)N[C@@H](C(=O)N3CCN(CC4CCN(CC(=O)N5CCN(C(=O)c6cc(Cc7n[nH]c(=O)c8ccccc78)ccc6F)CC5)CC4)CC3)C3CCCCC3)c2)C1)CC1CCCCC1 ; O=C(N[C@@H](C(=O)N1CCN(CC2CCN(CC(=O)N3CCN(C(=O)c4cc(Cc5n[nH]c(=O)c6ccccc56)ccc4F)CC3)CC2)CC1)C1CCCCC1)c1cccc(C2CCCN(C(=O)CNCC3CCCCC3)C2)c1]

### Step 1: Synthesis of Intermediate 1-3

To a solution of Intermediate 1-1 (150 mg, 163.73 μmol, 1 eq) and Intermediate 1-2 (53.31 mg, 196.48 μmol, 1.2 eq) in DMF (1.5 mL) was added HOAt (11.14 mg, 81.87 μmol, 11.45 μL, 0.5 eq), NMM (82.80 mg, 818.66 μmol, 90.01 μL, 5 eq) and EDCI (62.78 mg, 327.46 μmol, 2 eq) at 25° C., then the mixture was stirred at 25° C. for 1 hr. The reaction mixture was concentrated under reduced pressure to give a crude product. The crude product was purified by reversed-phase HPLC (0.1% FA condition) and lyophilized to give Intermediate 1-3 (120 mg, 102.61 μmol, 62.67% yield, 100% purity) as a white solid. LCMS (Method D): Rt=0.407 min, [M+H]⁺=1069.6.

### Step 2: Synthesis of I-348

A solution of Intermediate 1-3 (10 mg, 8.55 μmol, 1 eq) in DCM (0.2 mL) was added TFA (61.40 mg, 538.49 μmol, 0.04 mL, 62.97 eq) at 25° C., then the mixture was stirred at 25° C. for 1 hr. The reaction mixture was concentrated under reduced pressure to give a crude product. The crude product was purified by reversed-phase HPLC (0.1% FA condition) and lyophilized to give I-348 (3.15 mg, 2.66 μmol, 31.13% yield, 100% purity, TFA salt) as a white solid. LCMS (Method D): Rt=0.328 min, [M+H]⁺=1069.5. ¹H NMR (400 MHz, METHANOL-d4) δ=8.54-8.44 (m, 1H), 8.40-8.34 (m, 1H), 7.99-7.92 (m, 1H), 7.92-7.80 (m, 2H), 7.80-7.69 (m, 2H), 7.55-7.33 (m, 5H), 7.17 (t, J=9.2 Hz, 1H), 4.74-4.42 (m, 4H), 4.39 (s, 2H), 4.09-3.90 (m, 2H), 3.89-3.64 (m, 8H), 3.63-3.48 (m, 4H), 3.48-3.34 (m, 2H), 3.27-3.19 (m, 1H), 3.16 (d, J=1.2 Hz, 3H), 2.94-2.69 (m, 4H), 2.57-2.19 (m, 8H), 2.11-2.03 (m, 1H), 2.00-1.53 (m, 18H), 1.39-1.19 (m, 8H), 1.17-0.95 (m, 4H). ¹⁹F NMR (400 MHz, METHANOL-d4) δ=−120.779.

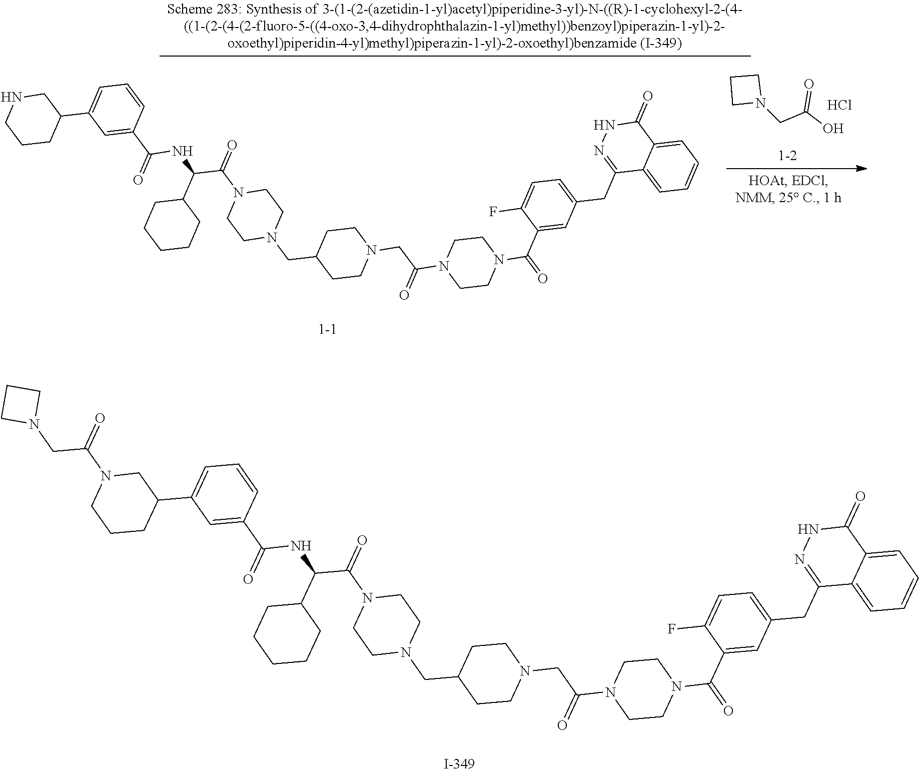 [SMILES: O=C(N[C@@H](C(=O)N1CCN(CC2CCN(CC(=O)N3CCN(C(=O)c4cc(Cc5n[nH]c(=O)c6ccccc56)ccc4F)CC3)CC2)CC1)C1CCCCC1)c1cccc(C2CCCNC2)c1 ; O=C(O)CN1CCC1 ; Cl ; O=C(N[C@@H](C(=O)N1CCN(CC2CCN(CC(=O)N3CCN(C(=O)c4cc(Cc5n[nH]c(=O)c6ccccc56)ccc4F)CC3)CC2)CC1)C1CCCCC1)c1cccc(C2CCCN(C(=O)CN3CCC3)C2)c1]

To a solution of intermediate 1-1 (100 mg, 109.15 μmol, 1 eq) and intermediate 1-2 (19.86 mg, 130.99 μmol, 1.2 eq, HCl salt) in DMF (1 mL) was added EDCI (41.85 mg, 218.31 μmol, 2 eq), NMM (55.20 mg, 545.77 μmol, 60.01 μL, 5 eq) and HOAt (7.43 mg, 54.58 μmol, 7.63 μL, 0.5 eq) at 25° C., then the mixture was stirred at 25° C. for 1 hr. The reaction mixture was concentrated under reduced pressure to give a crude product. The crude product was purified by reversed-phase HPLC (0.1% FA condition) and lyophilized to give I-349 (44.82 mg, 42.31 μmol, 38.76% yield, FA salt) as a yellow solid. LCMS (Method D): Rt=0.282 min, [M+H]⁺=1013.5. ¹H NMR (400 MHz, METHANOL-d4) δ=8.44 (s, 2H), 8.37 (d, J=7.2 Hz, 1H), 7.94 (d, J=4.8 Hz, 1H), 7.91-7.80 (m, 2H), 7.80-7.67 (m, 2H), 7.58-7.42 (m, 3H), 7.37 (s, 1H), 7.17 (t, J=9.2 Hz, 1H), 4.52 (s, 2H), 4.45-4.27 (m, 4H), 4.27-4.11 (m, 4H), 4.04-3.50 (m, 13H), 3.49-3.35 (m, 3H), 3.22 (d, J=12.0 Hz, 2H), 3.00-2.63 (m, 4H), 2.60-2.39 (m, 6H), 2.34-2.20

(m, 2H), 2.10-2.03 (m, 1H), 2.01-1.84 (m, 6H), 1.83-1.73 (m, 3H), 1.73-1.57 (m, 3H), 1.51-1.35 (m, 2H), 1.34-1.19 (m, 3H), 1.18-1.03 (m, 2H). ¹⁹F NMR (376 MHz, METHANOL-d4) δ=−120.673.

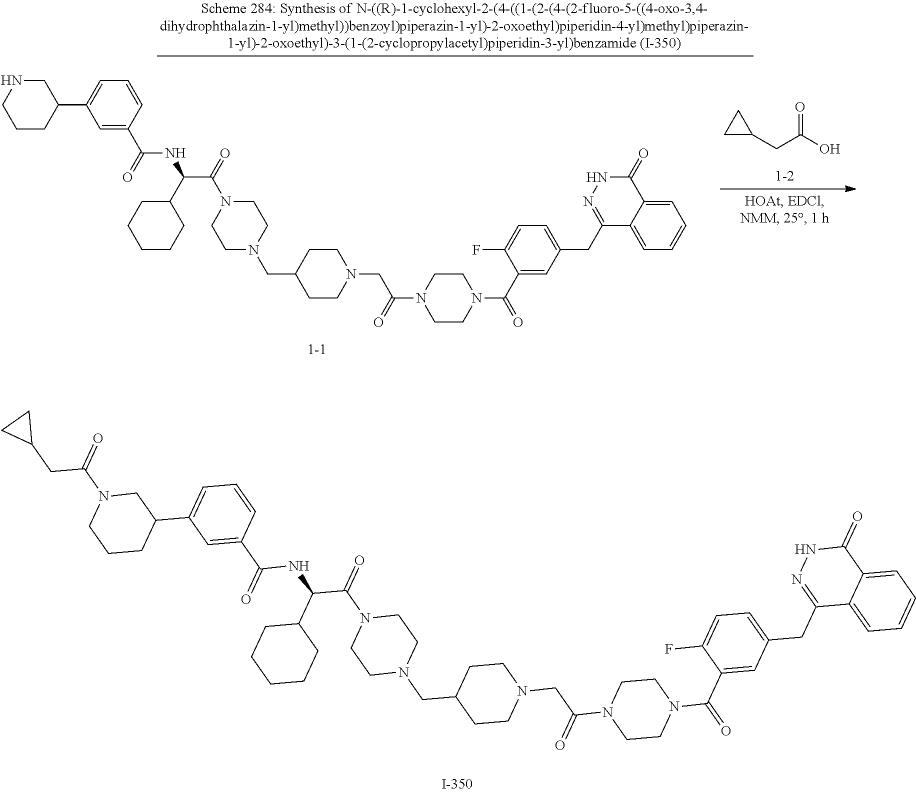 [SMILES: O=C(N[C@@H](C(=O)N1CCN(CC2CCN(CC(=O)N3CCN(C(=O)c4cc(Cc5n[nH]c(=O)c6ccccc56)ccc4F)CC3)CC2)CC1)C1CCCCC1)c1cccc(C2CCCNC2)c1 ; O=C(O)CC1CC1 ; O=C(N[C@@H](C(=O)N1CCN(CC2CCN(CC(=O)N3CCN(C(=O)c4cc(Cc5n[nH]c(=O)c6ccccc56)ccc4F)CC3)CC2)CC1)C1CCCCC1)c1cccc(C2CCCN(C(=O)CC3CC3)C2)c1]

To a solution of intermediate 1-1 (60 mg, 65.49 μmol, 1 eq) and 2-cyclopropylacetic acid (7.87 mg, 78.59 μmol, 1.2 eq) in DMF (0.6 mL) was added EDCI (25.11 mg, 130.99 μmol, 2 eq), HOAt (4.46 mg, 32.75 μmol, 4.58 μL, 0.5 eq) and NMM (33.12 mg, 327.46 μmol, 36.00 μL, 5 eq) at 25° C., then the mixture was stirred at 25° C. for 1 hr. The reaction mixture was concentrated under reduced pressure to give a crude product. The crude product was purified by reversed-phase HPLC (0.1% FA condition) and lyophilized to give I-350 (31 mg, 29.69 μmol, 45.33% yield, FA salt) as a white solid. LCMS (Method D): Rt=0.387 min, [M+H]⁺=998.5. ¹H NMR (400 MHz, METHANOL-d4) δ=8.26 (s, 1H), 8.16 (d, J=7.6 Hz, 1H), 7.73 (d, J=3.6 Hz, 1H), 7.70-7.59 (m, 2H), 7.55 (s, 1H), 7.52-7.47 (m, 1H), 7.28 (d, J=3.6 Hz, 2H), 7.26-7.20 (m, 1H), 7.16 (t, J=6.4 Hz, 1H), 6.96 (t, J=8.8 Hz, 1H), 4.69 (d, J=8.0 Hz, 1H), 4.45-4.38 (m, 1H), 4.18 (s, 2H), 3.85-3.73 (m, 1H), 3.70-3.20 (m, 13H), 3.05-2.91 (m, 4H), 2.63-2.46 (m, 2H), 2.42-2.23 (m, 5H), 2.22-2.12 (m, 3H), 2.06 (t, J=7.2 Hz, 2H), 1.91-1.81 (m, 1H), 1.77-1.43 (m, 12H), 1.22-0.98 (m, 5H), 0.95-0.77 (m, 3H), 0.40-0.25 (m, 2H), 0.05-0.10 (m, 2H). F NMR (376 MHz, METHANOL-d4) δ=−120.785.

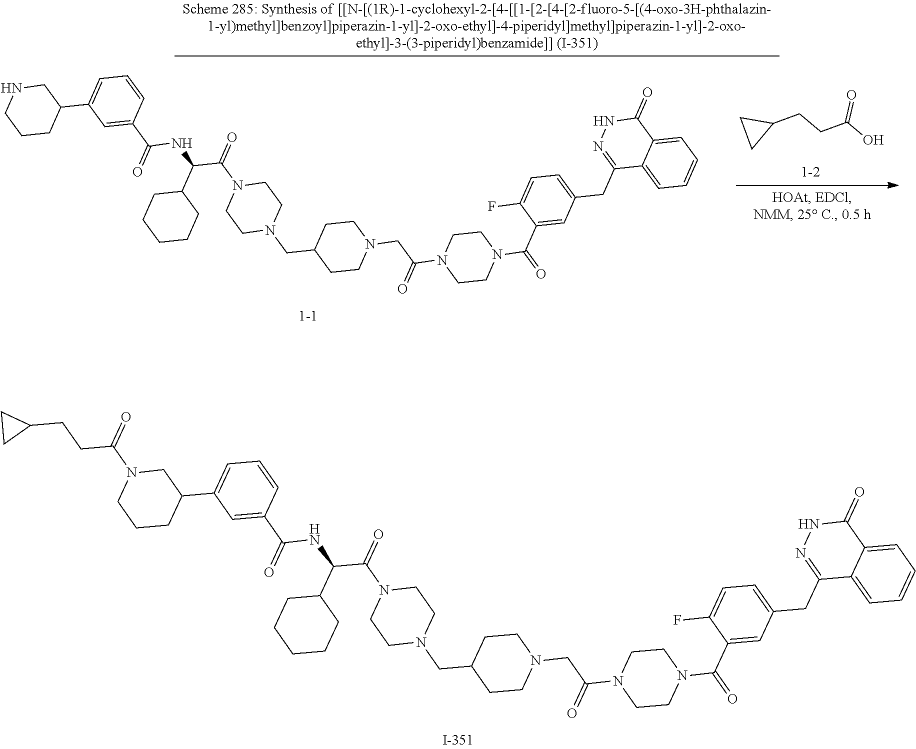 [SMILES: O=C(N[C@@H](C(=O)N1CCN(CC2CCN(CC(=O)N3CCN(C(=O)c4cc(Cc5n[nH]c(=O)c6ccccc56)ccc4F)CC3)CC2)CC1)C1CCCCC1)c1cccc(C2CCCNC2)c1 ; O=C(O)CCC1CC1 ; O=C(N[C@@H](C(=O)N1CCN(CC2CCN(CC(=O)N3CCN(C(=O)c4cc(Cc5n[nH]c(=O)c6ccccc56)ccc4F)CC3)CC2)CC1)C1CCCCC1)c1cccc(C2CCCN(C(=O)CCC3CC3)C2)c1]

To a solution of intermediate 1-1 (60 mg, 65.49 μmol, 1 eq) and intermediate 1-2 (8.97 mg, 78.59 μmol, 1.2 eq) in DMF (0.6 mL) was added EDCI (37.67 mg, 196.48 μmol, 3 eq), NMM (33.12 mg, 327.46 μmol, 36.00 μL, 5 eq) and HOAt (8.91 mg, 65.49 μmol, 9.16 μL, 1 eq). The mixture was stirred at 25° C. for 0.5 hr. The reaction mixture was poured into H₂O (2 mL), and extracted with EA (2 mL*3). The combined organic layers were dried over Na₂SO₄, filtered and concentrated under reduced pressure to give a residue. The residue was purified by prep-HPLC (column: Phenomenex Luna C18 150*25 mm*10 um; mobile phase: [water (FA)-ACN]; gradient: 12%-42% B over 10 min) and concentrated under reduced pressure to remove ACN, and lyophilized to give I-351 (16.12 mg, 15.23 μmol, 23.26% yield, 100% purity, FA salt) as a white solid. LCMS (Method D): Rt=0.367 min, (M+H)⁺=1012.5. ¹H NMR (400 MHz, METHANOL-d4) δ=8.46 (s, 1H), 8.41-8.31 (m, 1H), 8.01-7.92 (m, 1H), 7.91-7.79 (m, 2H), 7.79-7.67 (m, 2H), 7.55-7.46 (m, 2H), 7.46-7.40 (m, 1H), 7.37 (t, J=6.8 Hz, 1H), 7.17 (t, J=9.2 Hz, 1H), 4.92 (s, 2H), 4.66-4.54 (m, 2H), 4.39 (s, 2H), 4.05 (t, J=13.2 Hz, 1H), 3.98-3.65 (m, 8H), 3.62-3.42 (m, 4H), 3.35 (s, 2H), 3.29-3.12 (m, 3H), 2.89-2.66 (m, 2H), 2.63-2.46 (m, 6H), 2.44-2.35 (m, 1H), 2.27 (t, J=7.2 Hz, 2H), 2.05 (d, J=12.8 Hz, 1H), 1.84 (s, 8H), 1.72-1.58 (m, 3H), 1.56-1.49 (m, 2H), 1.47-1.34 (m, 2H), 1.34-1.20 (m, 3H), 1.19-1.02 (m, 2H), 0.84-0.67 (m, 1H), 0.51-0.41 (m, 2H), 0.11-0.04 (m, 2H). F NMR (400 MHz, METHANOL-d4) δ=−120.770.

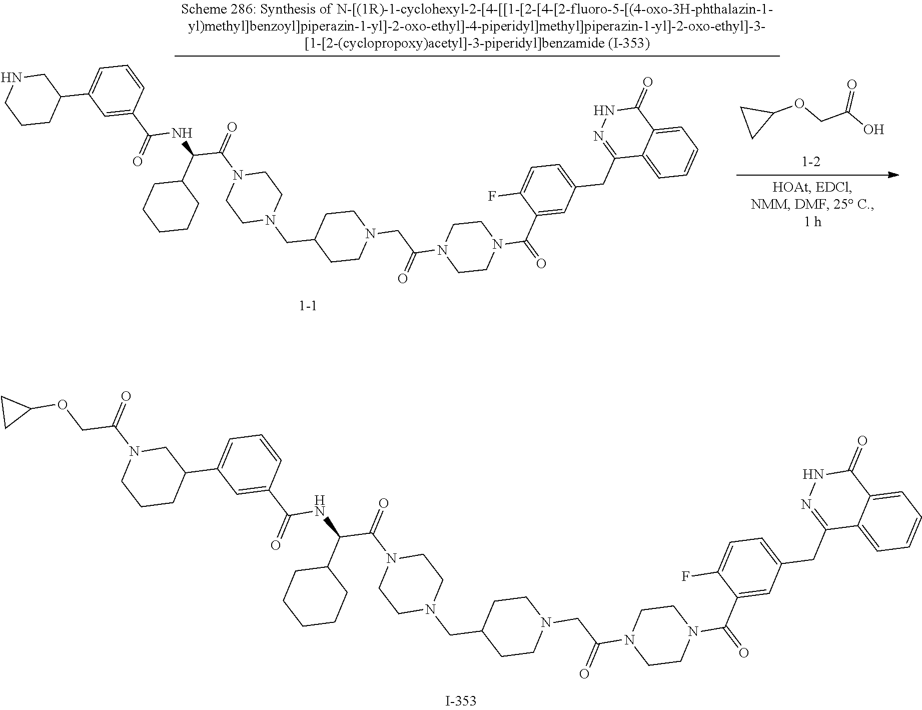 [SMILES: O=C(N[C@@H](C(=O)N1CCN(CC2CCN(CC(=O)N3CCN(C(=O)c4cc(Cc5n[nH]c(=O)c6ccccc56)ccc4F)CC3)CC2)CC1)C1CCCCC1)c1cccc(C2CCCNC2)c1 ; O=C(O)COC1CC1 ; O=C(N[C@@H](C(=O)N1CCN(CC2CCN(CC(=O)N3CCN(C(=O)c4cc(Cc5n[nH]c(=O)c6ccccc56)ccc4F)CC3)CC2)CC1)C1CCCCC1)c1cccc(C2CCCN(C(=O)COC3CC3)C2)c1]

To a solution of intermediate 1-2 (6.34 mg, 54.58 μmol, 1 eq) in DMF (0.5 mL) was added EDCI (31.39 mg, 163.73 μmol, 3 eq), HOAt (7.43 mg, 54.58 μmol, 7.63 μL, 1 eq) and NMM (27.60 mg, 272.89 μmol, 30.00 μL, 5 eq). Then intermediate 1-1 (50 mg, 54.58 μmol, 1 eq) was added. The mixture was stirred at 25° C. for 1 hr. The reaction mixture was concentrated to give a residue. The residue was purified by prep-HPLC (FA condition), the eluent was concentrated under reduced pressure to remove ACN and lyophilized to give I-353 (35.73 mg, 33.70 μmol, 61.75% yield, FA salt) as a white solid. LCMS (Method D): Rt=0.348 min, (M+H)=1014.4. SFC: Rt=1.768 min, 2.080 min. ¹H NMR (400 MHz, METHANOL-d4) δ=8.51 (s, 1H), 8.37 (d, J=7.6 Hz, 1H), 7.98-7.91 (m, 1H), 7.90-7.80 (m, 2H), 7.76 (s, 1H), 7.71 (d, J=6.8 Hz, 1H), 7.49 (s, 2H), 7.44-7.42 (m, 1H), 7.37 (d, J=5.2 Hz, 1H), 7.17-7.16 (m, 1H), 4.90-4.88 (m, 1H), 4.59-4.53 (m, 1H), 4.39 (s, 2H), 4.32-4.17 (m, 2H), 3.92 (d, J=12.4 Hz, 1H), 3.79 (d, J=4.4 Hz, 2H), 3.76-3.65 (m, 5H), 3.52 (s, 4H), 3.48-3.39 (m, 2H), 3.34 (s, 1H), 3.25-2.97 (m, 4H), 2.90-2.77 (m, 1H), 2.75-2.66 (m, 1H), 2.53-2.42 (m, 3H), 2.41-2.28 (m, 3H), 2.27-2.21 (m, 2H), 2.05 (d, J=12.0 Hz, 1H), 1.93-1.76 (m, 8H), 1.73-1.61 (m, 4H), 1.35-1.20 (m, 5H), 1.15-1.06 (m, 2H), 0.65-0.55 (m, 2H), 0.50-0.48 (m, 2H). ¹⁹F NMR (376 MHz, MeOD-d6) δ=−120.778.

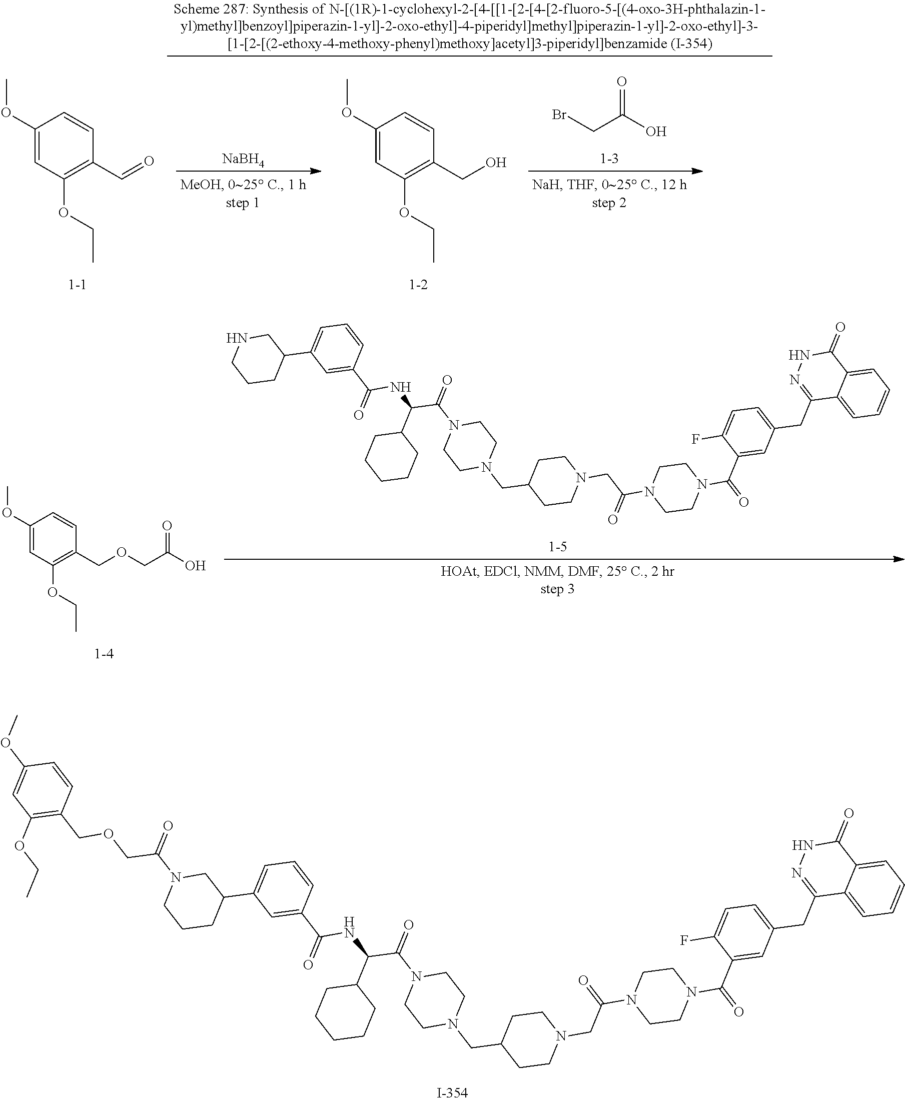

### Step 1: Synthesis of Intermediate 1-2.

To a solution of Intermediate 1-1 (500 mg, 2.77 mmol, 1 eq) in MeOH (5 mL) was added NaBH₄ (190 mg, 5.02 mmol, 1.81 eq) at 0° C. The mixture was stirred at 25° C. for 1 hr. The reaction mixture was quenched by addition of aq. NH₄Cl (2 mL) at 25° C. The reaction mixture diluted with H₂O (4 mL) and extracted with EA (5 mL*2). The combined organic layers were dried over Na₂SO₄, filtered and concentrated under reduced pressure to give Intermediate 1-2 (550 mg, crude) as a yellow solid, which was used in the next step without further purification. LCMS (Method C): Retention time: 0.625 min, (M+H-18)⁺=405.0. ¹H NMR (400 MHz, METHANOL-d4) δ=7.22 (d, J=8.0 Hz, 1H), 6.51-6.45 (m, 2H), 4.55 (s, 2H), 4.07-4.00 (m, 2H), 3.77 (s, 3H), 1.44-1.36 (m, 3H).

### Step 2: Synthesis of Intermediate 1-4.

To a solution of Intermediate 1-2 (300 mg, 1.65 mmol, 1 eq) in THF (5 mL) was added NaH (395.10 mg, 9.88 mmol, 60% purity, 6 eq) at 0° C. The mixture was stirred at 25° C. for 0.5 hr. Then to the mixture was added Intermediate 1-3 (228.76 mg, 1.65 mmol, 118.29 μL, 1 eq) and it was stirred at 25° C. for 11.5 hr. The reaction mixture was quenched by aq. NH₄Cl (4 mL) and extracted with EA (4 mL*2). The combined organic layers were dried over Na₂SO₄, filtered and concentrated under reduced pressure to give a residue. The crude product was purified by reversed-phase HPLC (0.1% FA condition). The eluent was concentrated under reduced pressure to give Intermediate 1-4 (150 mg, 624.34 μmol, 37.92% yield) as a white solid. ¹H NMR (400 MHz, METHANOL-d4) δ=7.23 (d, J=8.3 Hz, 1H), 6.52-6.47 (m, 2H), 4.57 (s, 2H), 4.07-4.00 (m, 4H), 3.78 (s, 3H), 1.43-1.37 (m, 3H).

### Step 3: Synthesis of I-354

To a solution of Intermediate 1-5 (39.34 mg, 163.73 μmol, 1.5 eq) in DMF (1 mL) was added Intermediate 1-4 (100 mg, 109.15 μmol, 1 eq), EDCI (62.78 mg, 327.46 μmol, 3 eq), HOAt (14.86 mg, 109.15 μmol, 15.27 μL, 1 eq) and NMM (33.12 mg, 327.46 μmol, 36.00 μL, 3 eq). The mixture was stirred at 25° C. for 0.5 hr. The mixture was filtered. The filtrate was purified by HPLC (column: CD04-Welch Utimate C18 150*25*7 um; mobile phase: [water (TFA)-ACN]; gradient: 27%-57% B over 10 min). The eluent was added into aq. NaHCO₃ (1.5 mL) and concentrated under reduced pressure to remove ACN. The remaining liquid was extracted with EA (5 mL*2). The combined organic layers were dried over Na₂SO₄, filtered and concentrated under reduced pressure to give a residue. The residue was purified by reversed-phase HPLC (0.1% NH₃·H₂O). The eluent was concentrated under reduced pressure to remove ACN and then lyophilized to give I-354 (3.86 mg, 3.37 μmol, 3.09% yield, 99.4% purity) as a white solid. LCMS (Method D): Retention time: 0.383 min, (M+H)⁺=1138.6. SFC: Retention time=4.393 min, 4.907 min. ¹H NMR (400 MHz, METHANOL-d₄) δ=8.40-8.33 (m, 1H), 7.98-7.92 (m, 1H), 7.91-7.80 (m, 2H), 7.77-7.67 (m, 2H), 7.52-7.40 (m, 2H), 7.39-7.28 (m, 2H), 7.26-7.12 (m, 2H), 6.54-6.47 (m, 1H), 6.41 (d, J=5.6 Hz, 1H), 4.93-4.90 (m, 1H), 4.60-4.49 (m, 5H), 4.38 (s, 2H), 4.29-4.01 (m, 3H), 4.00-3.81 (m, 3H), 3.77 (d, J=10.8 Hz, 4H), 3.74-3.65 (m, 4H), 3.60-3.48 (m, 3H), 3.29-3.18 (m, 2H), 3.16-3.03 (m, 1H), 2.98-2.84 (m, 2H), 2.80-2.61 (m, 2H), 2.55-2.40 (m, 3H), 2.37 (br s, 1H), 2.20 (d, J=6.0 Hz, 2H), 2.14-1.97 (m, 3H), 1.93-1.66 (m, 10H), 1.65-1.49 (m, 2H), 1.45-1.03 (m, 11H). ¹⁹F NMR (376 MHz, METHANOL-d₄) δ=−120.772.

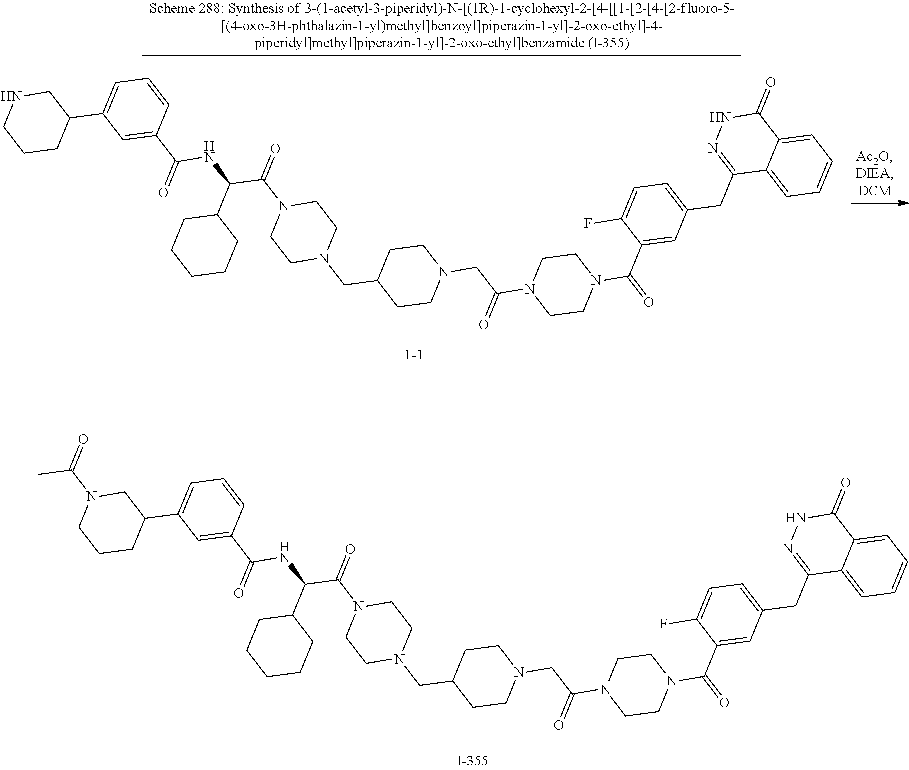 [SMILES: O=C(N[C@@H](C(=O)N1CCN(CC2CCN(CC(=O)N3CCN(C(=O)c4cc(Cc5n[nH]c(=O)c6ccccc56)ccc4F)CC3)CC2)CC1)C1CCCCC1)c1cccc(C2CCCNC2)c1 ; CC(=O)N1CCCC(c2cccc(C(=O)N[C@@H](C(=O)N3CCN(CC4CCN(CC(=O)N5CCN(C(=O)c6cc(Cc7n[nH]c(=O)c8ccccc78)ccc6F)CC5)CC4)CC3)C3CCCCC3)c2)C1]

To a solution of Intermediate 1-1 (50 mg, 54.58 μmol, 1 eq) in DCM (1 mL) was added DIEA (14.11 mg, 109.15 μmol, 19.01 μL, 2 eq) and Ac₂O (6.69 mg, 65.49 μmol, 6.15 μL, 1.2 eq). The mixture was stirred at 25° C. for 1 hr. The reaction mixture was quenched by addition of NH₃·H₂O (0.05 mL) at 25° C., and then diluted with H₂O (1 mL) and extracted with DCM (1 mL*3). The combined organic layers were concentrated under reduced pressure to give a residue. The residue was purified by prep-HPLC (FA condition), and the eluent was concentrated and lyophilized to give I-355 (31.28 mg, 31.08 μmol, 56.94% yield, 99.7% purity) as a white solid. LCMS (Method D): Retention time: 0.337 min, (M+H)⁺=958.9. SFC: Retention time: 2.558, 2.854 min. ¹H NMR (400 MHz, CHLOROFORM-d) δ=10.37-10.00 (m, 1H), 8.49-8.43 (m, 1H), 8.14 (s, 1H), 7.82-7.74 (m, 2H), 7.71 (s, 2H), 7.68-7.63 (m, 1H), 7.43-7.29 (m, 4H), 7.09-7.00 (m, 2H), 5.09-5.00 (m, 1H), 4.76-4.66 (m, 1H), 4.28 (s, 2H), 3.86 (d, J=12.8 Hz, 1H), 3.79-3.65 (m, 5H), 3.64-3.51 (m, 4H), 3.37-3.24 (m, 2H), 3.23-3.17 (m, 2H), 3.17-3.05 (m, 1H), 2.94-2.83 (m, 2H), 2.78-2.69 (m, 1H), 2.65-2.52 (m, 1H), 2.50-2.35 (m, 4H), 2.21 (d, J=7.2 Hz, 2H), 2.17-2.11 (m, 3H), 2.10 (s, 3H), 1.78-1.70 (m, 9H), 1.69-1.45 (m, 4H), 1.28-1.07 (m, 7H), ¹⁹F NMR (376 MHz, METHANOL-d4) δ=−117.67.

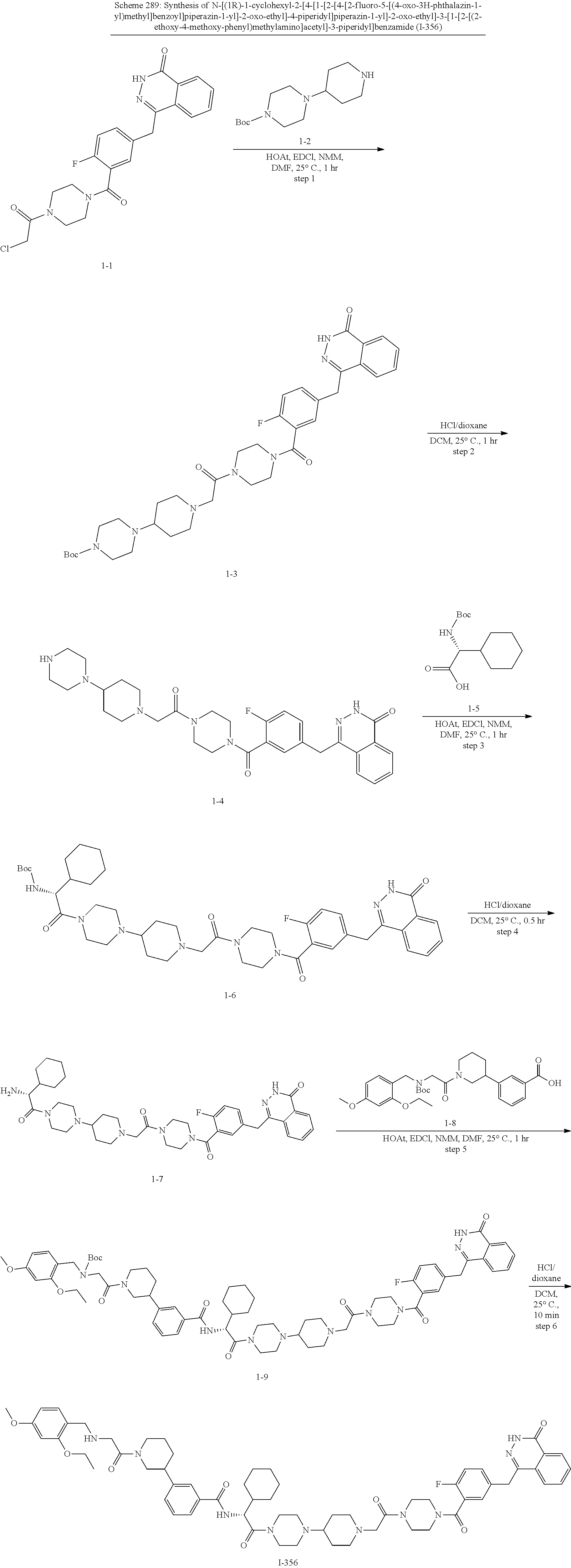

### Step 1: Synthesis of Intermediate 1-3

To a solution of intermediate 1-1 (400 mg, 903.20 μmol, 1 eq) and intermediate 1-2 (243.31 mg, 903.20 μmol, 1 eq) in ACN (4 mL) was added DIEA (350.20 mg, 2.71 mmol, 471.96 μL, 3 eq). The mixture was stirred at 60° C. for 2 hr. The mixture was washed with water (6 mL) and extracted with EA (8 mL*3). The combined organic layers were dried over sodium sulfate, filtered and concentrated under reduced pressure to give a residue. The crude product was used in the next step without further purification. Intermediate 1-3 (600 mg, 781.31 μmol, 86.50% yield, 88% purity) was obtained as a yellow solid. LCMS (Method D): Rt=0.273 min, (M+H)⁺=676.3. ¹H NMR (400 MHz, METHANOL-d4) δ=8.37 (d, J=7.6 Hz, 1H), 7.95 (d, J=6.4 Hz, 1H), 7.91-7.80 (m, 2H), 7.49 (d, J=5.2 Hz, 1H), 7.41-7.34 (m, 1H), 7.16-7.14 (m, 1H), 4.39 (s, 2H), 3.81-3.65 (m, 4H), 3.59-3.50 (m, 2H), 3.42 (s, 4H), 3.34 (s, 1H), 3.25-3.18 (m, 2H), 3.00-2.91 (m, 2H), 2.53 (s, 4H), 2.30-2.22 (m, 1H), 2.08-2.03 (m, 2H), 1.85 (d, J=0.8 Hz, 2H), 1.61-1.49 (m, 2H), 1.46 (s, 9H), 1.27 (d, J=7.2 Hz, 1H).

### Step 2: Synthesis of Intermediate 1-4

To a solution of intermediate 1-3 (550 mg, 813.86 μmol, 1 eq) in DCM (3 mL) was added HCl/dioxane (2 M, 3 mL, 7.37 eq). The mixture was stirred at 25° C. for 1 hr. The reaction mixture was concentrated in vacuo. The crude product was used in the next step without further purification. Intermediate 1-4 (550 mg, crude, HCl salt) was obtained as a yellow solid. LCMS (Method D): Rt=0.225 min, (M+H)⁺=576.3.

### Step 3: Synthesis of Intermediate 1-6

To a solution of intermediate 1-5 (210.19 mg, 816.81 μmol, 1 eq) in DMF (5 mL) was added EDCI (469.75 mg, 2.45 mmol, 3 eq) and HOAt (111.18 mg, 816.81 μmol, 114.26 μL, 1 eq). Then intermediate 1-4 (500 mg, 816.81 μmol, 1 eq, HCl salt) and NMM (413.09 mg, 4.08 mmol, 449.01 μL, 5 eq) was added. The mixture was stirred at 25° C. for 1 hr. The mixture was washed with water (10 mL) and extracted with EA (15 mL*3). The combined organic layers were dried over sodium sulfate, filtered and concentrated under reduced pressure to give a residue. The residue was purified by flash silica gel chromatography (ISCO; 40 g SepaFlash Silica Flash Column, Eluent of 0˜100% MeOH/EA @60 mL/min) and then the eluent was concentrated in vacuo to give Intermediate 1-6 (400 mg, 480.99 μmol, 58.89% yield, 98% purity) as a yellow solid. LCMS (Method D): Rt=0.323 min, (M+H)⁺=815.4. ¹H NMR (400 MHz, METHANOL-d4) δ=8.37 (d, J=7.6 Hz, 1H), 7.98-7.92 (m, 1H), 7.91-7.80 (m, 2H), 7.54-7.45 (m, 1H), 7.38 (s, 1H), 7.16-7.14 (m, 1H), 4.39 (s, 2H), 3.83-3.62 (m, 7H), 3.61-3.45 (m, 4H), 3.35 (s, 1H), 3.29 (s, 1H), 3.22 (s, 1H), 3.03-2.94 (m, 2H), 2.61 (d, J=15.6 Hz, 5H), 2.32-2.22 (m, 1H), 2.18-2.07 (m, 2H), 1.86 (d, J=6.4 Hz, 2H), 1.76 (d, J=10.4 Hz, 4H), 1.66-1.64 (m, 2H), 1.60 (d, J=12.0 Hz, 2H), 1.52 (d, J=12.0 Hz, 1H), 1.43 (s, 9H), 1.24-1.22 (m, 4H). ¹⁹F NMR (376 MHz, MeOD-d6) δ=−120.700.

### Step 4: Synthesis of Intermediate 1-7

To a solution of intermediate 1-6 (350 mg, 429.46 μmol, 1 eq) in DCM (2 mL) was added HCl/dioxane (2 M, 2 mL). The mixture was stirred at 25° C. for 0.5 hr. The reaction mixture was concentrated in vacuo. The crude product was used in the next step without further purification. Intermediate 1-7 (350 mg, crude, HCl salt) was obtained as a yellow solid. LCMS (Method D): Rt=0.241 min, (M+H)⁺=715.5. SFC: Rt=0.910 min, 1.466 min. ¹H NMR (400 MHz, METHANOL-d4) δ=8.37 (d, J=7.6 Hz, 1H), 7.95 (d, J=3.6 Hz, 1H), 7.92-7.81 (m, 2H), 7.54-7.47 (m, 1H), 7.38-7.37 (m, 1H), 7.17-7.14 (m, 1H), 4.39 (s, 2H), 3.86-3.70 (m, 7H), 3.68 (s, 2H), 3.62 (s, 3H), 3.59-3.50 (m, 3H), 3.39 (s, 4H), 3.29-3.19 (m, 2H), 2.59-2.25 (m, 4H), 1.86-1.69 (m, 6H), 1.68-1.50 (m, 1H), 1.48-1.36 (m, 1H), 1.36-1.29 (m, 2H), 1.28-1.10 (m, 4H). ¹⁹F NMR (376 MHz, MeOD-d6) δ=−120.725.

### Step 5: Synthesis of Intermediate 1-9

To a solution of intermediate 1-8 (140.18 mg, 266.19 μmol, 1 eq) in DMF (2 mL) was added EDCI (153.09 mg, 798.58 μmol, 3 eq) and HOAt (36.23 mg, 266.19 μmol, 37.24 μL, 1 eq). Then intermediate 1-7 (200 mg, 266.19 μmol, 1 eq, HCl salt) and NMM (134.62 mg, 1.33 mmol, 146.33 μL, 5 eq) was added. The mixture was stirred at 25° C. for 1 hr. The mixture was washed with water (3 mL) and extracted with EA (5 mL*3). The combined organic layers were dried over sodium sulfate, filtered and concentrated under reduced pressure to give a residue. The residue was purified by flash silica gel chromatography (ISCO; 20 g SepaFlash Silica Flash Column, Eluent of 0˜100% MeOH/EA@50 mL/min) and then the eluent was concentrated in vacuo to give Intermediate 1-9 (95 mg, 77.65 μmol, 29.17% yield) as yellow gum. LCMS (Method D): Rt=0.418 min, (M+H)⁺=1223.7. SFC: Rt=3.036 min, 3.210 min.

### Step 6: Synthesis of I-356

To a solution of intermediate 1-9 (80 mg, 65.39 μmol, 1 eq) in DCM (1 mL) was added HCl/dioxane (2 M, 0.2 mL). The mixture was stirred at 25° C. for 10 min. The reaction mixture was concentrated in vacuo. The residue was purified by prep-HPLC (0.1% NH₃·H₂O condition), and the eluent was concentrated under reduced pressure to remove ACN and lyophilized to give I-356 (24.42 mg, 20.28 μmol, 31.02% yield, 93.3% purity) as a white solid. LCMS (Method D): Rt=0.324 min, (M+H)⁺=1123.6. SFC: Rt=2.718 min, 3.855 min. ¹H NMR (400 MHz, METHANOL-d4) δ=8.37 (d, J=7.6 Hz, 1H), 7.95 (d, J=6.0 Hz, 1H), 7.90-7.80 (m, 2H), 7.78-7.67 (m, 2H), 7.52-7.40 (m, 3H), 7.37 (s, 1H), 7.21-7.10 (m, 2H), 6.58-6.35 (m, 2H), 4.64-4.46 (m, 3H), 4.38 (s, 2H), 4.11-4.00 (m, 2H), 3.89-3.88 (m, 1H), 3.83-3.64 (m, 12H), 3.59-3.43 (m, 5H), 3.24-3.17 (m, 2H), 3.14-3.05 (m, 1H), 2.99-2.91 (m, 2H), 2.77-2.58 (m, 5H), 2.57-2.49 (m, 1H), 2.32-2.22 (m, 1H), 2.14-2.00 (m, 3H), 1.91-1.73 (m, 8H), 1.72-1.65 (m, 2H), 1.63-1.48 (m, 3H), 1.46-1.39 (m, 3H), 1.33-1.23 (m, 4H), 1.17-1.05 (m, 2H). ¹⁹F NMR (376 MHz, MeOD-d6) δ=−120.722.

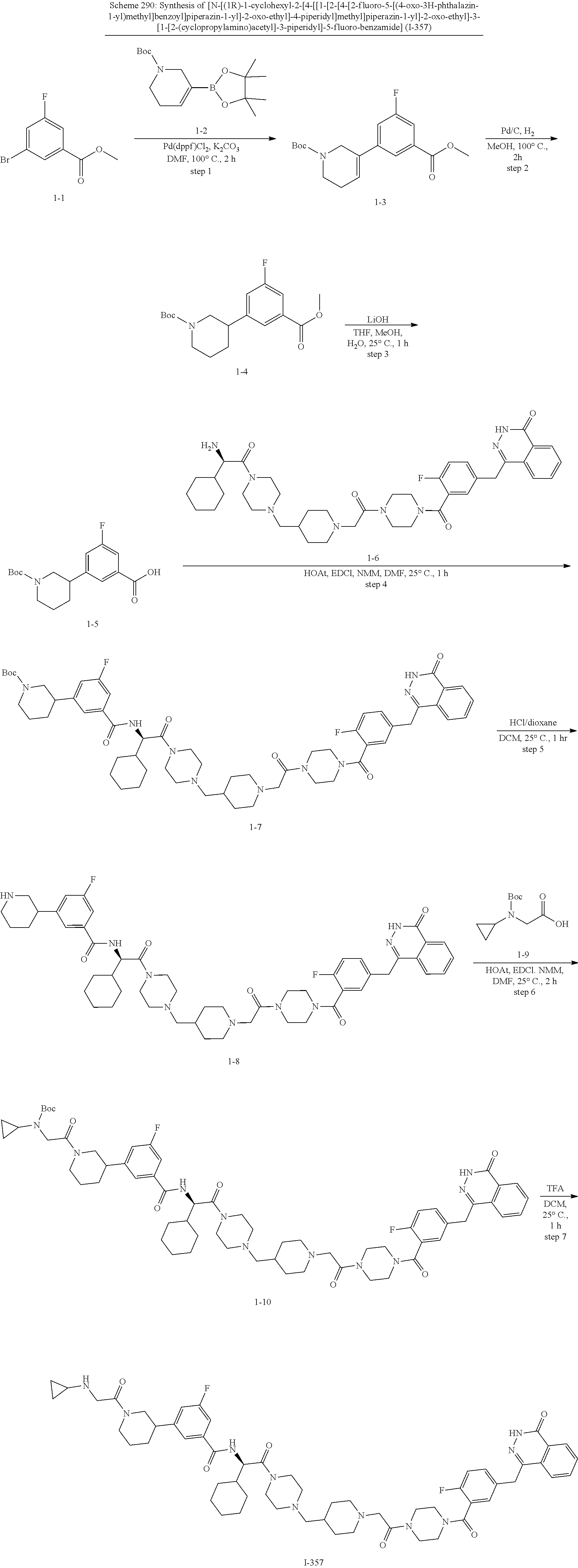

### Step 1: Synthesis of Intermediate 1-3

A mixture of intermediate 1-1 (500 mg, 2.15 mmol, 1 eq), intermediate 1-2 (796.13 mg, 2.57 mmol, 1.2 eq), cyclopentyl(diphenyl)phosphane;dichloropalladium;iron (157.00 mg, 214.56 μmol, 0.1 eq), and K₂CO₃ (593.07 mg, 4.29 mmol, 2 eq) in DMF (5 mL) was degassed and purged with N₂ 3 times, and then the mixture was stirred at 100° C. for 2 hrs under N₂ atmosphere. The residue was diluted with H₂O (5 mL) and extracted with EA (5 mL*3) dried over Na₂SO₄, filtered and concentrated under reduced pressure to give a residue. The residue was purified by flash silica gel chromatography (ISCO; 12 g SepaFlash Silica Flash Column, Eluent of 0˜100% Petroleum ether/Ethyl acetate@60 mL/min). The eluent was concentrated under reduced pressure to give Intermediate 1-3 (700 mg, 2.09 mmol, 97.28% yield) as yellow oil. LCMS (Method D): Rt=0.576 min, [M+Na]⁺=357.9. ¹H NMR (400 MHz, METHANOL-d4) δ=7.85 (br s, 1H), 7.61-7.59 (m, 1H), 7.40 (br d, J=10.0 Hz, 1H), 6.46-6.31 (m, 1H), 4.27 (br s, 2H), 3.93 (s, 3H), 3.57 (brt, J=5.6 Hz, 2H), 2.41-2.27 (m, 2H), 1.50 (s, 9H).

### Step 2: Synthesis of Intermediate 1-4

To a solution of intermediate 1-3 (600 mg, 1.79 mmol, 1 eq) in MeOH (7 mL) was added Pd/C (300 mg, 1.79 mmol, 10% purity, 1.00 eq) under N₂ atmosphere. The suspension was degassed and purged with H₂ 3 times. The mixture was stirred under H₂ at 25° C. for 1 hr. The reaction was diluted with MeOH (5 mL*5), filtered and concentrated under reduced pressure to give intermediate 1-4 (600 mg, 1.78 mmol, 99.40% yield) as yellow oil which was used in the next step without further purification. LCMS (Method D): Rt=0.487 min, [M+Na]⁺=360.1.

### Step 3: Synthesis of Intermediate 1-5

To a solution of intermediate 1-4 (300 mg, 889.19 μmol, 1 eq) in THF (1 mL), H₂O (1 mL) and MeOH (1 mL) was added LiOH·H₂O (111.94 mg, 2.67 mmol, 3 eq). The mixture was stirred at 25° C. for 1 hr. The mixture was adjusted to pH=6 with sat. citric acid and then extracted with EA (5 mL*3). The combined organic layers were dried over Na₂SO₄, filtered and concentrated under reduced pressure to give intermediate 1-5 (280 mg, 865.91 μmol, 97.38% yield) as yellow oil. LCMS (Method D): Rt=0.492 min, [M+Na]⁺=345.9.

### Step 4: Synthesis of Intermediate 1-7

To a solution of intermediate 1-5 (280 mg, 865.91 μmol, 1 eq) in DMF (3 mL) was added EDCI (829.98 mg, 4.33 mmol, 5 eq), NMM (875.84 mg, 8.66 mmol, 952.00 μL, 10 eq) and HOAt (235.72 mg, 1.73 mmol, 242.26 μL, 2 eq) then intermediate 1-6 (729.01 mg, 952.50 μmol, 1.1 eq, HCl salt) was added. The mixture was stirred at 25° C. for 1 hr. The reaction mixture was diluted with H₂O (5 mL) and extracted with EA (5 mL*3), dried over Na₂SO₄, filtered and concentrated under reduced pressure to give a residue. The residue was purified by flash silica gel chromatography (ISCO 20 g SepaFlash Silica Flash Column, Eluent of 0˜100% Dichloromethane/Methanol @60 mL/min). The eluent was concentrated under reduced pressure to give Intermediate 1-7 (700 mg, 676.83 μmol, 78.16% yield) as a yellow solid. LCMS (Method D): Rt=0.393 min, [M+H]⁺=1034.4. ¹H NMR (400 MHz, METHANOL-d4) δ=8.37 (d, J=8.0 Hz, 1H), 7.95 (br d, J=8.4 Hz, 1H), 7.91-7.79 (m, 2H), 7.59 (s, 1H), 7.53-7.42 (m, 2H), 7.38 (br s, 1H), 7.24 (br d, J=9.6 Hz, 1H), 7.16 (t, J=8.8 Hz, 1H), 5.49 (s, 2H), 4.38 (s, 2H), 4.09 (br d, J=12.8 Hz, 2H), 3.88-3.62 (m, 7H), 3.61-3.47 (m, 3H), 3.27-3.18 (m, 2H), 2.96-2.88 (m, 2H), 2.80-2.70 (m, 1H), 2.56-2.31 (m, 4H), 2.23-2.19 (m, 2H), 2.15-1.98 (m, 3H), 1.87 (br d, J=14.0 Hz, 2H), 1.82-1.72 (m, 6H), 1.72-1.52 (m, 5H), 1.47 (s, 9H), 1.38-0.99 (m, 9H). SFC: Retention time: 0.934.

### Step 5: Synthesis of Intermediate 1-8

To a solution of intermediate 1-7 (400 mg, 386.76 μmol, 1 eq) in DCM (1.5 mL) was added HCl/dioxane (2 M, 2.5 mL, 12.93 eq). The mixture was stirred at 25° C. for 1 hr. The mixture was concentrated under reduced pressure to give intermediate 1-8 (380 mg, crude, HCl salt) as a yellow oil which was used in the next step without further purification. LCMS (Method D): Rt=0.279 min, [M+H]⁺=934.5.

### Step 6: Synthesis of Intermediate 1-10

To a solution of intermediate 1-9 (101.13 mg, 469.82 μmol, 1.2 eq) in DMF (4 mL) was added EDCI (375.27 mg, 1.96 mmol, 5 eq), NMM (396.01 mg, 3.92 mmol, 430.44 μL, 10 eq) and HOAt (106.58 mg, 783.03 μmol, 109.54 μL, 2 eq). The intermediate 1-8 (380 mg, 391.52 μmol, 1 eq, HCl salt) was added. The mixture was stirred at 25° C. for 1 hr. The mixture was poured into 5 mL of H₂O and then extracted with EA (5 mL*3). The combined organic layers were dried over Na₂SO₄, filtered and concentrated under reduced pressure to give intermediate 1-10 (400 mg, 353.56 μmol, 90.30% yield) as a yellow oil which was used in the next step without further purification. LCMS (Method D): Rt=0.368 min, [M+H]⁺=1132.5.

### Step 7: Synthesis of I-357

A solution of intermediate 1-10 (400 mg, 353.56 μmol, 1 eq) in TFA (1 mL) and DCM (3 mL) was stirred at 25° C. for 1 hr. The reaction mixture was concentrated under reduced pressure to give a residue. The crude product was purified by prep-HPLC (column: CD04-Welch Utimate C18 150*25*7 um;mobile phase: [water(TFA)-ACN]; gradient: 13%-43% B over 10 min). The eluent was concentrated under lyophilization to give I-357 (175 mg, 168.29 μmol, 47.60% yield, 99.1% purity) as a white solid. LCMS (Method D): Rt=0.295 min, [M+H]⁺=1031.4. ¹H NMR (400 MHz, METHANOL-d4) δ=8.71-8.60 (m, 1H), 8.37 (d, J=7.6 Hz, 1H), 7.99-7.93 (m, 1H), 7.92-7.79 (m, 2H), 7.67-7.60 (m, 1H), 7.55-7.44 (m, 2H), 7.40-7.26 (m, 1H), 7.28 (br d, J=9.2 Hz, 1H), 7.18 (t, J=9.2 Hz, 1H), 4.85-4.75 (m, 2H), 4.63-4.49 (m, 1H), 4.39 (s, 2H), 4.35-4.28 (m, 2H), 4.25 (br d, J=12.4 Hz, 2H), 4.22-4.13 (m, 1H), 3.98-3.65 (m, 7H), 3.59-3.48 (m, 3H), 3.43-3.33 (m, 5H), 3.28-3.04 (m, 6H), 2.98-2.86 (m, 1H), 2.84-2.69 (m, 2H), 2.35-2.21 (m, 1H), 2.17-1.86 (m, 7H), 1.83-1.58 (m, 7H), 1.39-1.04 (m, 6H), 0.98-0.86 (m, 4H). ¹⁹F NMR: (377 MHz, METHANOL-d₄) δ=−114.054, −120.674. SFC: Retention time: 1.942 min, 2.114 min.

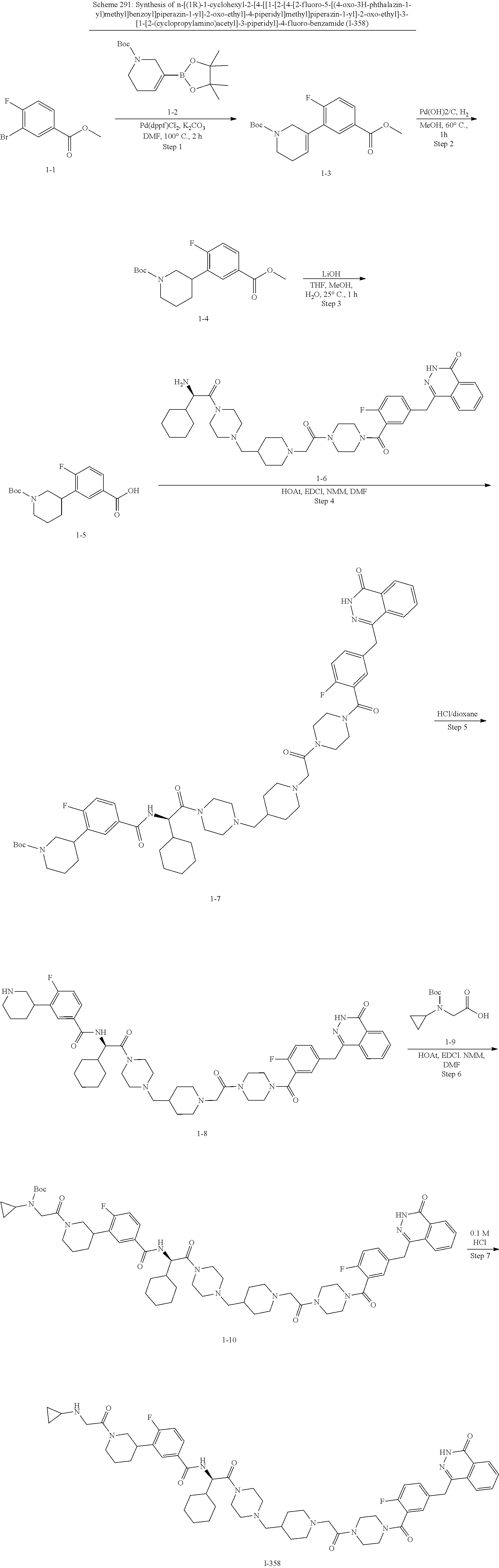

### Step 1: Synthesis of Intermediate 1-3

To a solution of intermediate 1-1 (1 g, 4.29 mmol, 1 eq) and intermediate 1-2 (1.59 g, 5.15 mmol, 1.2 eq) in dioxane (5 mL) and H₂O (1 mL) was added Pd(dppf)Cl₂ (313.99 mg, 429.12 μmol, 0.1 eq) and K₂CO₃ (1.19 g, 8.58 mmol, 2 eq). The mixture was stirred at 100° C. for 1 hr. The mixture was diluted with water (10 mL) and extracted with DCM (10 mL*3). The combined organic layers were dried over Na₂SO₄, filtered and concentrated in vacuo to give a residue. The residue was purified by column chromatography (SiO₂, PE:EA=20:1 to PE:EA=0:1), then the organic liquid was concentrated in vacuo to give Intermediate 1-3 (1.3 g, 3.68 mmol, 85.82% yield, 95% purity) as colorless oil. LCMS (Method D): Retention time=0.500 min, (M+Na)⁺=358.2.

### Step 2: Synthesis of Intermediate 1-4

To a solution of intermediate 1-3 (800 mg, 2.39 mmol, 1 eq) in MeOH (8 mL) was added Pd(OH)₂/C (200 mg, 20% purity) under N₂ atmosphere. The suspension was degassed and purged with H₂ 3 times. The mixture was stirred under H₂ (15 psi) at 60° C. for 1 hr. The reaction mixture was filtered and concentrated in vacuo to give a residue. The residue was used in the next step directly without further purification. Intermediate 1-4 (800 mg, crude) was obtained as colorless oil. LCMS (Method D): Retention time=0.584 min, (M+Na)⁺=360.1.

### Step 3: Synthesis of Intermediate 1-5

To a solution of intermediate 1-4 (800 mg, 2.37 mmol, 1 eq) in MeOH (2 mL), H₂O (2 mL) and THF (2 mL) was added LiOH·H₂O (199.01 mg, 4.74 mmol, 2 eq). The mixture was stirred at 25° C. for 0.5 hr. The reaction mixture was concentrated in vacuo to remove THF and MeOH to give a residue, and the residue was diluted with water (8 mL) and extracted with DCM (8 mL*3), then the aqueous phase was acidified with hydrochloric acid (1 M) to pH=5-6. The aqueous phase was extracted with DCM (8 mL*3). The combined organic layer was dried over Na₂SO₄, filtered and concentrated in vacuo to give a residue. The residue was used in the next step directly without further purification. Intermediate 1-5 (500 mg, 1.55 mmol, 65.21% yield) was obtained as colorless gum. LCMS (Method D): Retention time=0.504 min, (M+H)⁺=346.1.

### Step 4: Synthesis of Intermediate 1-7

To a solution of intermediate 1-5 (152.10 mg, 470.37 μmol, 1.2 eq) and intermediate 1-6 (300 mg, 391.97 μmol, 1 eq, HCl salt) in DMF (3 mL) was added EDCI (225.43 mg, 1.18 mmol, 3 eq) and HOAt (53.35 mg, 391.97 μmol, 54.83 μL, 1 eq), NMM (198.23 mg, 1.96 mmol, 215.47 μL, 5 eq). The mixture was stirred at 25° C. for 0.5 hr. The mixture was diluted with water (5 mL) and extracted with DCM (5 mL*3). The combined organic layer was dried over Na₂SO₄, filtered and concentrated in vacuo to give a residue. The residue was purified by column chromatography (SiO₂, PE:EA=10:1 to EA:MeOH=0:1), then the organic liquid was concentrated in vacuo to give Intermediate 1-7 (300 mg, 226.25 μmol, 57.72% yield, 78% purity) as yellow oil. LCMS (Method D): Retention time=0.387 min, (M+H)⁺=1034.3.

### Step 5: Synthesis of Intermediate 1-8

A solution of intermediate 1-7 (300 mg, 290.07 μmol, 1 eq) in HCl/dioxane (2 M, 2 mL, 13.79 eq) was stirred at 25° C. for 20 min. The mixture was diluted with water (5 mL) and extracted with DCM (5 mL*3). The combined organic layer was dried over Na₂SO₄, filtered and concentrated in vacuo to give a residue. The residue was purified by reverse phase HPLC (0.1% FA condition), then the eluent was concentrated in vacuo and lyophilized. Intermediate 1-8 (40 mg, 38.74 μmol, 13.36% yield, 94% purity, HCl salt) was obtained as a white solid. LCMS (Method D): Retention time=0.392 min, (M+H)⁺=934.7. SFC: Retention time=1.975 min, 2.074 min.

### Step 6: Synthesis of Intermediate 1-10

To a solution of intermediate 1-8 (40 mg, 42.82 μmol, 1 eq) and intermediate 1-9 (13.83 mg, 64.23 μmol, 1.5 eq) in DMF (0.5 mL) was added EDCI (24.63 mg, 128.46 μmol, 3 eq) and HOAt (5.83 mg, 42.82 μmol, 5.99 μL, 1 eq), NMM (21.66 mg, 214.10 μmol, 23.54 μL, 5 eq). The mixture was stirred at 25° C. for 0.5 hr. The mixture was diluted with water (5 mL) and extracted with DCM (5 mL*3). The combined organic layer was dried over Na₂SO₄, filtered and concentrated in vacuo to give a residue. The residue was purified by reverse phase HPLC (0.1% FA condition), then the residue was concentrated in vacuo and lyophilized. Intermediate 1-10 (20 mg, 16.79 μmol, 39.21% yield, 94.9% purity) was obtained as colorless gum. LCMS (Method D): Retention time=0.369 min, (M+H)⁺=1131.6.

### Step 7: Synthesis of I-358

To a solution of intermediate 1-10 (20 mg, 17.68 μmol, 1 eq) in DCM (0.3 mL) was added TFA (153.50 mg, 1.35 mmol, 0.1 mL, 76.15 eq). The mixture was stirred at 25° C. for 15 min. The reaction mixture was concentrated in vacuo to give a residue. The residue was purified by reversed phase HPLC (0.1% TFA condition), then the residue was concentrated in vacuo and lyophilized. I-358 (10.91 mg, 10.58 μmol, 59.85% yield, 100% purity) was obtained as a white solid. LCMS (Method D): Retention time=0.292 min, (M+H)⁺=1031.6. SFC: Retention time=3.540 min, 4.505 min. ¹H NMR (400 MHz, METHANOL-d4) δ=8.59-8.48 (m, 1H), 8.40-8.35 (m, 1H), 7.97-7.93 (m, 1H), 7.90-7.79 (m, 4H), 7.52 (d, J=2.4 Hz, 1H), 7.37-7.34 (m, 1H), 7.26-7.15 (m, 2H), 4.62-4.55 (m, 1H), 4.39 (s, 2H), 4.33-4.27 (m, 2H), 4.26-4.19 (m, 3H), 3.99-3.91 (m, 1H), 3.81-3.77 (m, 3H), 3.74-3.65 (m, 3H), 3.58-3.55 (m, 1H), 3.54-3.47 (m, 2H), 3.35 (d, J=3.2 Hz, 4H), 3.27-3.19 (m, 3H), 3.13-3.12 (m, 1H), 3.10-3.05 (m, 2H), 3.02-2.95 (m, 2H), 2.94-2.85 (m, 1H), 2.81-2.77 (m, 1H), 2.26-2.18 (m, 1H), 2.15-2.04 (m, 3H), 2.01-1.86 (m, 5H), 1.84-1.75 (m, 3H), 1.74-1.61 (m, 5H), 1.36-1.24 (m, 4H), 1.17-1.08 (m, 2H), 0.94-0.89 (m, 4H). ¹⁹F NMR (376 MHz, METHAN OL-d4) δ=−115.1, δ=−120.6.

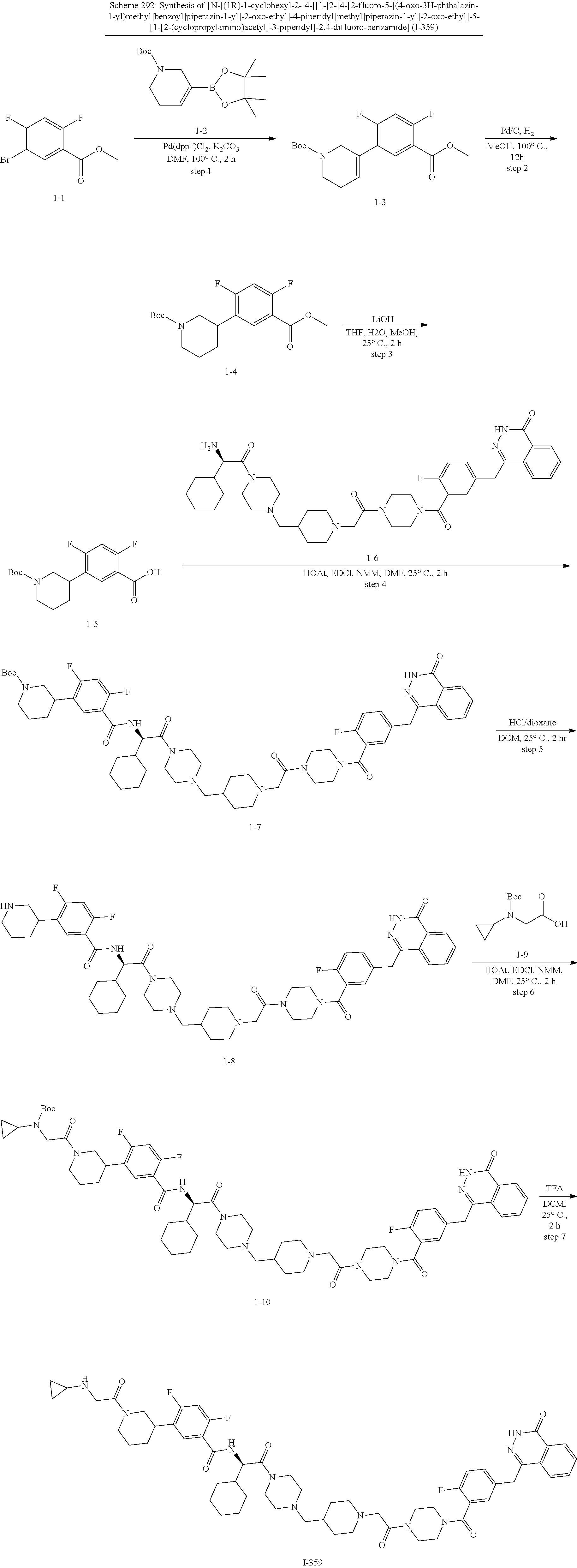

### Step 1: Synthesis of Intermediate 1-3

A mixture of intermediate 1-1 (500 mg, 1.99 mmol, 1 eq), intermediate 1-2 (923.84 mg, 2.99 mmol, 1.5 eq), Pd(dppf)Cl₂ (145.74 mg, 199.18 μmol, 0.1 eq), and K₂CO₃ (550.57 mg, 3.98 mmol, 2 eq) in DMF (5 mL) was degassed and purged with N₂ 3 times, and then the mixture was stirred at 100° C. for 1 hr under N₂ atmosphere. The residue was diluted with H₂O (15 mL) and extracted with EA (10 mL*3), dried over Na₂SO₄, filtered and concentrated under reduced pressure to give a residue. The residue was purified by flash silica gel chromatography (ISCO; 20 g SepaFlash Silica Flash Column, Eluent of 0˜100% Petroleum ether/Ethyl acetate @60 mL/min). The eluent was concentrated under reduced pressure to give Intermediate 1-3 (700 mg, 1.98 mmol, 99.46% yield) as a yellow oil. LCMS (Method D): Rt=0.506 min, [M+Na]⁺=376.0.

### Step 2: Synthesis of Intermediate 1-4

To a solution of intermediate 1-3 (200 mg, 566.00 μmol, 1 eq) in MeOH (1 mL) was added Pd/C (100 mg, 10% purity) under N₂ atmosphere. The suspension was degassed and purged with H₂ 3 times. The mixture was stirred under H₂ at 25° C. for 1 hr. The reaction was diluted with MeOH (5 mL*5), filtered and concentrated under reduced pressure to give a residue. The residue was purified by flash silica gel chromatography (ISCO; 20 g SepaFlash Silica Flash Column, Eluent of 0˜100% Petroleum ether/Ethyl acetate@60 mL/min). The eluent was concentrated under reduced pressure to give Intermediate 1-4 (190 mg, 534.65 μmol, 94.46% yield) as yellow oil. LCMS (Method D): Rt=0.563 min, [M+Na]⁺=377.9. ¹H NMR (400 MHz, METHANOL-d4) δ=7.92 (br t, J=8.4 Hz, 1H), 7.07 (br t, J=10.4 Hz, 1H), 4.15-3.99 (m, 2H), 3.94-3.74 (m, 3H), 3.01-2.72 (m, 3H), 1.97 (br d, J=10.0 Hz, 1H), 1.80 (br d, J=12.0 Hz, 2H), 1.65-1.52 (m, 1H), 1.50-1.39 (m, 9H).

### Step 3: Synthesis of Intermediate 1-5

To a solution of intermediate 1-4 (185 mg, 520.58 μmol, 1 eq) in THF (0.7 mL), H₂O (0.7 mL) and MeOH (0.7 mL) was added LiOH·H₂O (65.54 mg, 1.56 mmol, 3 eq). The mixture was stirred at 25° C. for 1 hr. The mixture was adjusted to pH=6 with sat. citric acid and then extracted with EA (5 mL*3). The combined organic layers were dried over Na₂SO₄, filtered and concentrated under reduced pressure to give intermediate 1-5 (190 mg, crude) as yellow oil, which was used in the next step without further purification. LCMS (Method D): Rt=0.482 min, [M+Na]⁺=363.9.

### Step 4: Synthesis of Intermediate 1-7

To a solution of intermediate 1-5 (170 mg, 498.02 μmol, 1 eq) in DMF (2 mL) was added EDCI (477.36 mg, 2.49 mmol, 5 eq), NMM (503.74 mg, 4.98 mmol, 547.54 μL, 10 eq) and HOAt (135.57 mg, 996.05 μmol, 139.34 μL, 2 eq), then intermediate 1-6 (399.31 mg, 547.83 μmol, 1.1 eq) was added. The mixture was stirred at 25° C. for 1 hr. The residue was diluted with H₂O (5 mL) and extracted with EA (5 mL*3), dried over Na₂SO₄, filtered and concentrated under reduced pressure to give a residue. The residue was purified by flash silica gel chromatography (ISCO; 12 g SepaFlash Silica Flash Column, Eluent of 0˜100% Dichloromethane/Methanol@60 mL/min). The eluent was concentrated under reduced pressure to give Intermediate 1-7 (150 mg, 142.55 μmol, 28.62% yield) as a yellow oil. LCMS (Method D): Rt=0.374 min, [M+H]⁺=1052.5.

### Step 5: Synthesis of Intermediate 1-8

A solution of intermediate 1-7 (150 mg, 142.55 μmol, 1 eq) in HCl/dioxane (2M, 1 mL) and DCM (0.5 mL) was stirred at 25° C. for 2 hr. The mixture was concentrated under reduced pressure to give a residue. The crude product was purified by reversed-phase HPLC (0.1% HCl condition). The eluent was concentrated under reduced pressure to remove ACN, then lyophilized to afford Intermediate 1-8 (90 mg, 94.53 μmol, 66.31% yield) as a white solid. LCMS (Method D): Rt=0.287 min, [M+H]⁺=952.5. SFC: Retention time: 1.232 min, 1.743 min.

### Step 6: Synthesis of Intermediate 1-10

To a solution of intermediate 1-9 (15.82 mg, 73.52 μmol, 1 eq) in DMF (0.5 mL) was added EDCI (70.47 mg, 367.60 μmol, 5 eq), NMM (74.36 mg, 735.21 μmol, 80.83 μL, 10 eq) and HOAt (20.01 mg, 147.04 μmol, 20.57 μL, 2 eq), then intermediate 1-8 (70 mg, 73.52 μmol, 1 eq) was added in. The mixture was stirred at 25° C. for 2 hr. The mixture was poured into 5 mL of H₂O and then extracted with EA (5 mL*3). The combined organic layers were dried over Na₂SO₄, filtered and concentrated under reduced pressure to give intermediate 1-10 (80 mg, 69.60 μmol, 94.67% yield) as yellow oil, which was used in the next step without further purification. LCMS (Method D): Rt=0.395 min, [M+H]⁺=1149.4.

### Step 7: Synthesis of I-359

A solution of intermediate 1-10 (80 mg, 69.60 μmol, 1 eq) in TFA (0.2 mL) and DCM (0.6 mL) was stirred at 25° C. for 1 hr. The reaction mixture was concentrated under reduced pressure to give a residue. The crude product was purified by reversed-phase HPLC (0.1% TFA condition). The eluent was concentrated under reduced pressure to remove ACN, then lyophilized to afford I-359 (28 mg, 26.69 mol, 38.34% yield, 100% purity) as a white solid. LCMS (Method D): Rt=0.289 min, [M+H]⁺=1149.5. ¹H NMR (400 MHz, METHANOL-d4) δ=8.45-8.32 (m, 1H), 7.99-7.91 (m, 1H), 7.91-7.80 (m, 2H), 7.78-7.67 (m, 1H), 7.58-7.47 (m, 1H), 7.38-7.34 (m, 1H), 7.22-7.07 (m, 2H), 4.62-4.53 (m, 1H), 4.39 (s, 2H), 4.32-4.15 (m, 5H), 4.04-3.91 (m, 1H), 3.87-3.62 (m, 7H), 3.61-3.45 (m, 3H), 3.36 (br s, 5H), 3.26-3.16 (m, 3H), 3.16-3.05 (m, 4H), 3.05-2.93 (m, 1H), 2.89-2.70 (m, 2H), 2.33-2.20 (m, 1H), 2.17-2.00 (m, 3H), 1.96-1.63 (m, 11H), 1.39-1.04 (m, 6H), 0.96-0.85 (m, 4H). ¹⁹F NMR: (377 MHz, METHANOL-d₄) δ=−111.141, −113.047, −120.755. SFC: Retention time: 3.882 min, 5.234 min.

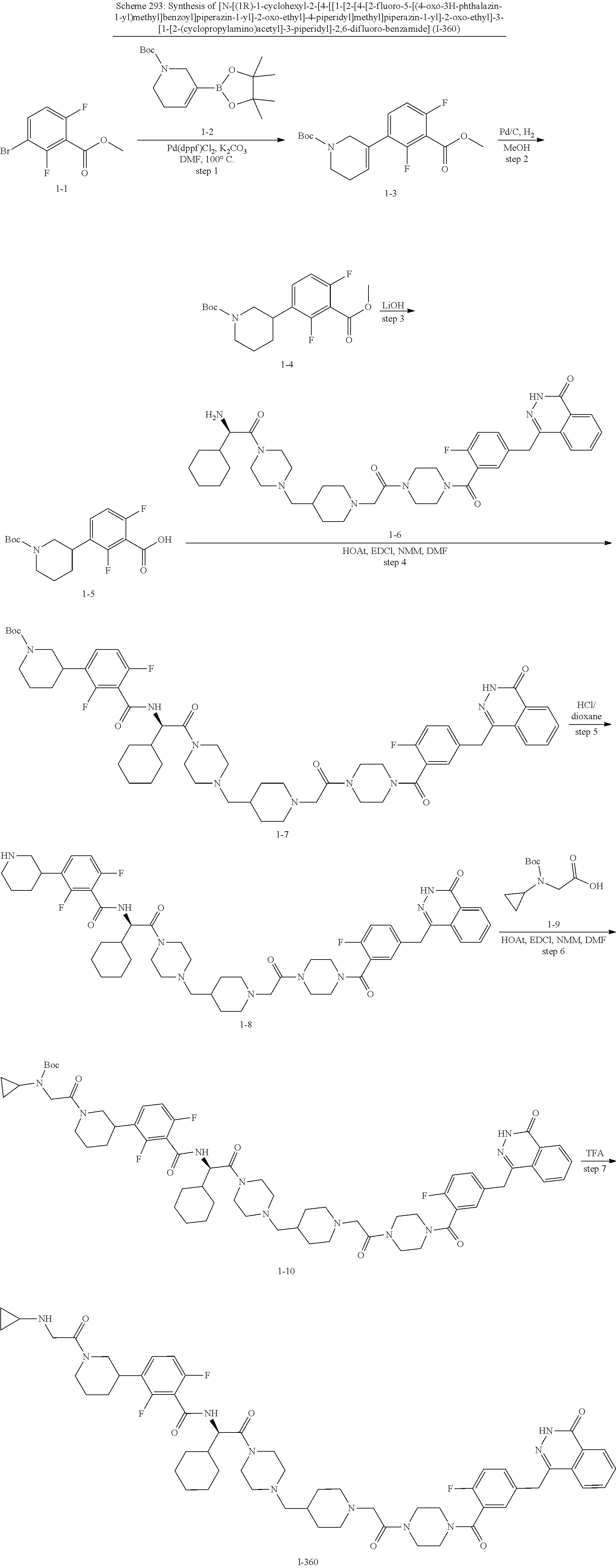

### Step 1: Synthesis of Intermediate 1-2

To a solution of Intermediate 1-1 (500 mg, 1.99 mmol, 1 eq) and Intermediate 1-2 (739.07 mg, 2.39 mmol, 1.2 eq) in DMF (5 mL) was added Pd(dppf)Cl₂ (145.74 mg, 199.18 μmol, 0.1 eq) and K₂CO₃ (550.57 mg, 3.98 mmol, 2 eq). The mixture was stirred at 100° C. for 2 h under N₂ atmosphere. The reaction mixture was diluted with H₂O (20 mL) at 25° C., and the mixture was extracted with EA (20 ml*3), and the combined organic phase was dried with anhydrous sodium sulfate, filtered and the filtrate was concentrated to give crude product. The crude product was purified by column chromatography (SiO₂, PE/EA=1/0 to 0/1) and the eluent was concentrated under reduced pressure to give Intermediate 1-3 (680 mg, 1.89 mmol, 94.68% yield, 98% purity) as a yellow oil. LCMS (Method D): Retention time: 0.572 min, (M+Na)⁺=376.0. ¹H NMR (400 MHz, METHANOL-d4) δ=7.51-7.44 (m, 1H), 7.09-7.04 (m, 1H), 6.05 (s, 1H), 4.20-4.12 (m, 2H), 3.93 (s, 3H), 3.57 (s, 2H), 2.33-2.30 (m, 2H), 1.48 (s, 9H)

### Step 2: Synthesis of Intermediate 1-4

To a solution of Intermediate 1-3 (680 mg, 1.92 mmol, 1 eq) in MeOH (14 mL) was added Pd/C (100 mg, 10% purity) under N₂ atmosphere. The system was purged with H₂, then kept under H₂ (15 psi) atmosphere. The system was allowed to reach 25° C. and stirred for 2 hrs. The mixture was filtered and concentrated under reduced pressure to give Intermediate 1-4 (660 mg, crude) as a yellow oil. LCMS (Method D): Retention time: 0.575 min, (M+Na)⁺=378.1.

### Step 3: Synthesis of Intermediate 1-5

To a solution of Intermediate 1-4 (660 mg, 1.86 mmol, 1 eq) in MeOH (2.2 mL), THF (2.2 mL) and H₂O (2.2 mL) was added LiOH·H₂O (233.80 mg, 5.57 mmol, 3 eq). The mixture was stirred at 25° C. for 1 h. The mixture was to added 10 ml of H₂O and adjusted to pH 5-6 with 1 M aq. HCl, and the mixture was extracted with EA (20 ml*3), and the combined organic phase was dried with anhydrous sodium sulfate, filtered and filtrate was concentrated to give Intermediate 1-5 (620 mg, crude) as a white solid. LCMS (Method D): Retention time: 0.492 min, (M+Na)⁺=364.0.

### Step 4: Synthesis of Intermediate 1-7

To a solution of Intermediate 1-5 (100 mg, 292.96 μmol, 1 eq) in DMF (2 mL) was added EDCI (168.48 mg, 878.87 μmol, 3 eq), NMM (148.16 mg, 1.46 mmol, 161.04 μL, 5 eq), Intermediate 1-6 (224.22 mg, 292.96 μmol, 1 eq, HCl salt) and HOAt (39.87 mg, 292.96 μmol, 40.98 μL, 1 eq). The mixture was stirred at 25° C. for 1 h. The reaction mixture was washed with H₂O (5 mL) at 25° C., and mixture was extracted with EA (5 ml*3), and the combined organic phase was dried with anhydrous sodium sulfate, filtered and the filtrate was concentrated to give crude product. The crude product was purified by column chromatography (SiO₂, EA/MeOH=1/0 to 0/1) and the eluent was concentrated under reduced pressure to give crude product. The crude product was further purified by prep-HPLC (column: Welch Xtimate C18 150*30 mm*5 um; mobile phase: [water(FA)-ACN]; gradient: 21%-51% B over 10 min) and the eluent was lyophilized to give Intermediate 1-7 (220 mg, 194.44 μmol, 66.37% yield, 93% purity) as a white solid. LCMS (Method D): Retention time: 0.363 min, (M+H)⁺=1052.2. SFC: Retention time: 1.698 min, 1.801 min.

### Step 5: Synthesis of Intermediate 1-8

To a solution of Intermediate 1-7 (180 mg, 171.07 μmol, 1 eq) in DCM (0.9 mL) was added HCl/dioxane (2 M, 1.80 mL, 21.04 eq). The mixture was stirred at 25° C. for 0.5 h. The mixture was concentrated under reduced pressure to give Intermediate 1-8 (180 mg, crude, HCl salt) as a white solid. LCMS (Method D): Retention time: 0.302 min, (M+H)⁺=952.5.

### Step 6: Synthesis of Intermediate 1-10

To a solution of Intermediate 1-8 (180 mg, 182.08 μmol, 1 eq, HCl salt) in DMF (2 mL) was added EDCI (104.71 mg, 546.24 μmol, 3 eq), NMM (92.08 mg, 910.40 μmol, 100.09 μL, 5 eq), Intermediate 1-9 (39.19 mg, 182.08 μmol, 1 eq) and HOAt (24.78 mg, 182.08 μmol, 25.47 μL, 1 eq). The mixture was stirred at 25° C. for 0.5 h. The crude product was purified by prep-HPLC (0.1% FA condition) and the eluent was lyophilized to give Intermediate 1-10 (120 mg, 99.19 μmol, 54.47% yield, 95% purity) as a white solid. LCMS (Method D): Retention time: 0.362 min, (M+H)⁺=1149.2. SFC: Retention time: 0.701 min, 1.158 min.

### Step 7: Synthesis of I-360

To a solution of Intermediate 1-10 (50 mg, 43.50 μmol, 1 eq) in DCM (0.6 mL) was added TFA (307.00 mg, 2.69 mmol, 0.2 mL, 61.89 eq). The mixture was stirred at 25° C. for 0.5 h. The mixture was concentrated under reduced pressure to give a residue. Then 10 ml of H₂O was added into the product, and the mixture was lyophilized to give I-360 (42.91 mg, 35.73 μmol, 82.14% yield, 96.874% purity, TFA salt) as a white solid. LCMS (Method D): Retention time: 0.285 min, (M+H)⁺=1049.4. SFC: Retention time: 2.819 min, 3.331 min. ¹H NMR (400 MHz, METHANOL-d4) δ=8.37 (d, J=7.6 Hz, 1H), 8.00-7.92 (m, 1H), 7.91-7.81 (m, 2H), 7.56-7.42 (m, 2H), 7.41-7.33 (m, 1H), 7.17 (t, J=9.2 Hz, 1H), 7.11-6.99 (m, 1H), 4.61-4.52 (m, 1H), 4.39 (s, 2H), 4.34-4.16 (m, 5H), 4.07-3.94 (m, 1H), 3.84-3.65 (m, 6H), 3.61-3.47 (m, 3H), 3.36 (s, 5H), 3.26-3.05 (m, 6H), 3.04-2.94 (m, 1H), 2.94-2.65 (m, 3H), 2.38-2.22 (m, 1H), 2.19-2.08 (m, 2H), 2.07-1.54 (m, 13H), 1.35-1.10 (m, 6H), 0.94-0.88 (m, 4H).

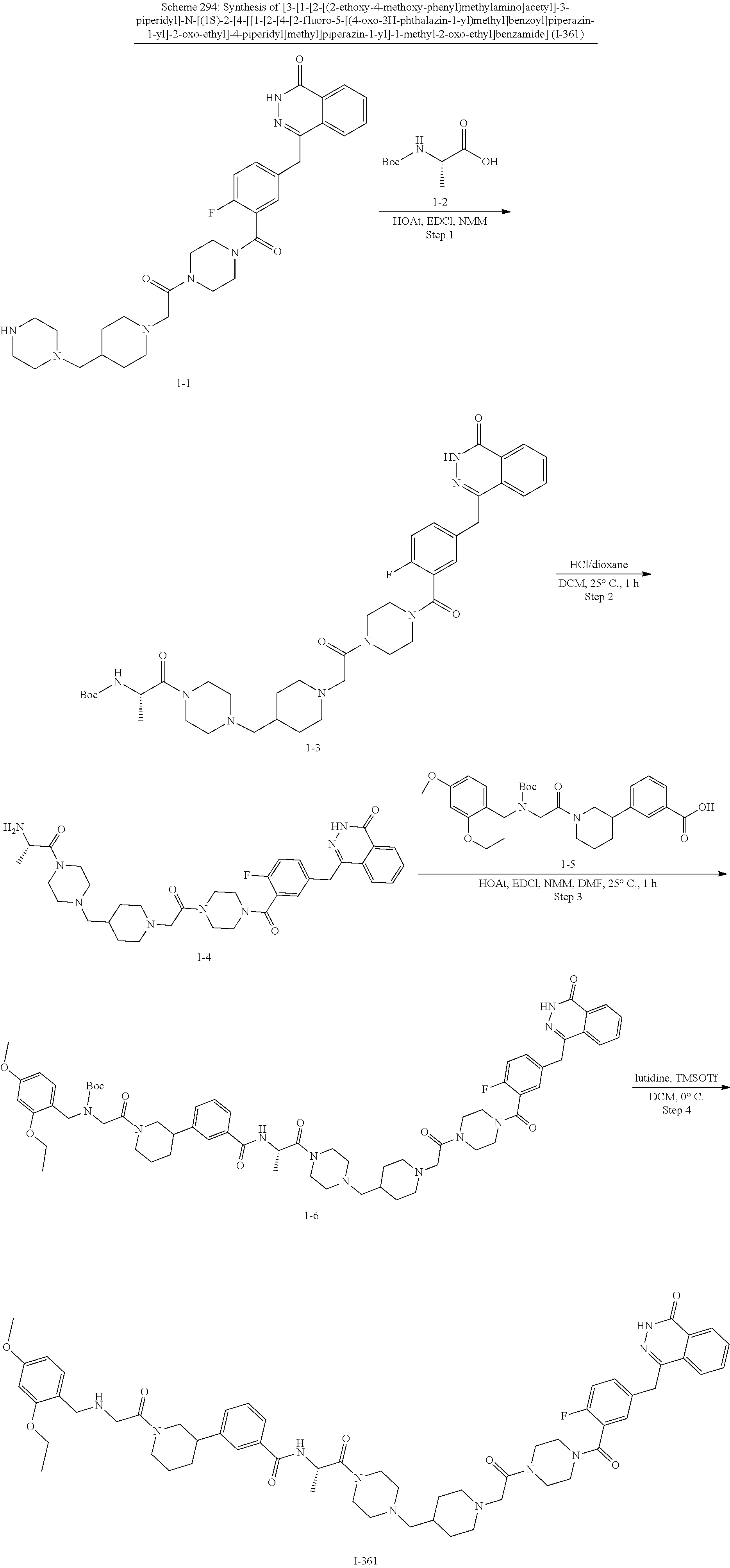

### Step 1: Synthesis of Intermediate 1-3.

To a mixture of Intermediate 1-1 (200 mg, 319.41 μmol, 1 eq, HCl salt) and Intermediate 1-2 (72.52 mg, 383.29 μmol, 1.2 eq) in DMF (2 mL) was added EDCI (183.69 mg, 958.22 μmol, 3 eq), HOAt (43.47 mg, 319.41 μmol, 44.68 μL, 1 eq) and NMM (161.53 mg, 1.60 mmol, 175.58 μL, 5 eq). The mixture was stirred at 25° C. for 1 hr. The reaction was poured into H₂O (3 mL) and extracted with DCM (5 mL*3). The combined organic layers were dried over Na₂SO₄, filtered and concentrated under reduced pressure to give the crude product. The crude product was purified by flash silica gel chromatography (ISCO; 20 g SepaFlash Silica Flash Column, Eluent of 0˜65% Ethyl acetate/Methanol gradient @40 mL/min) and concentrated under reduced pressure to give Intermediate 1-3 (210 mg, 270.47 μmol, 84.68% yield, 98% purity) as a yellow solid. LCMS (Method D): Retention time: 0.245 min, (M+H)⁺=761.3. ¹H NMR (400 MHz, METHANOL-d4) δ=8.56 (d, J=3.6 Hz, 1H), 8.39-8.35 (m, 1H), 8.24 (d, J=8.4 Hz, 1H), 7.99-7.92 (m, 1H), 7.92-7.87 (m, 1H), 7.39-7.35 (m, 2H), 4.64-4.56 (m, 2H), 4.39 (s, 2H), 4.18 (s, 1H), 4.14-4.08 (m, 1H), 3.84 (d, J=2.4 Hz, 3H), 3.77 (d, J=4.8 Hz, 1H), 3.71 (d, J=4.0 Hz, 1H), 3.55 (s, 3H), 3.10 (s, 2H), 2.57-2.44 (m, 4H), 2.32 (d, J=6.0 Hz, 2H), 2.08-2.04 (m, 1H), 2.00-1.97 (m, 1H), 1.93-1.84 (m, 8H), 1.56-1.49 (m, 2H), 1.44 (s, 9H), 1.31-1.28 (m, 1H). ¹⁹F NMR (376 MHz, METHANOL-d₄) δ=−120.8.

### Step 2: Synthesis of Intermediate 1-4.

A mixture of intermediate 1-3 (200 mg, 262.85 μmol, 1 eq) in DCM (1 mL) was added HCl/dioxane (2 M, 1.00 mL, 7.61 eq) at 25° C. was stirred at 25° C. for 0.5 hr. The reaction mixture was concentrated to give the crude product. The crude product was used for the next step directly. Intermediate 1-4 (180 mg, 258.16 μmol, 98.22% yield, HCl salt) was obtained as a white solid. LCMS (Method D): Retention time: 0.195 min, (M+H)⁺=661.3.

### Step 3: Synthesis of Intermediate 1-6.

To a mixture of intermediate 1-4 (180 mg, 258.16 μmol, 1 eq, HCl salt) in DMF (2 mL) was added intermediate 1-5 (135.95 mg, 258.16 μmol, 1 eq), EDCI (148.47 mg, 774.48 μmol, 3 eq), HOAt (35.14 mg, 258.16 μmol, 36.11 μL, 1 eq) and NMM (130.56 mg, 1.29 mmol, 141.91 μL, 5 eq) at 25° C., and the mixture was stirred at 25° C. for 1 hr. The reaction mixture was concentrated to give a residue. The residue was purified by reversed-phase HPLC (0.1% FA condition) and lyophilized to give Intermediate 1-6 (100 mg, 85.52 μmol, 33.12% yield) as a yellow solid. LCMS (Method D): Retention time: 0.429 min, (M+H)⁺=1169.6.

### Step 4: Synthesis of I-361

To a mixture of intermediate 1-6 (30 mg, 25.65 μmol, 1 eq) in DCM (0.5 mL) was added 2,6-dimethylpyridine (824.69 g, 7.70 μmol, 8.96e−1 μL, 0.3 eq) and trimethylsilyl trifluoromethanesulfonate (8.55 mg, 38.48 μmol, 6.95 μL, 1.5 eq) at 0° C., the mixture was stirred at 0° C. for 0.1 hr. The reaction mixture was concentrated to give a residue. The residue was purified by reversed-phase HPLC (0.1% NH₃·H₂O) and lyophilized to give I-361 (6.78 mg, 5.84 μmol, 22.76% yield, 92.0% purity) as a white solid. LCMS (Method D): Retention time: 0.282 min, (M+H)⁺=1069.5. SFC: Retention time: 5.460 min, 7.805 min. ¹H NMR (400 MHz, METHANOL-d4) δ=8.37 (d, J=7.2 Hz, 1H), 7.98-7.93 (m, 1H), 7.91-7.80 (m, 2H), 7.77 (s, 1H), 7.74 (t, J=7.2 Hz, 1H), 7.52-7.41 (m, 3H), 7.40-7.35 (m, 1H), 7.23-7.13 (m, 2H), 6.56 (d, J=9.6 Hz, 1H), 6.53-6.47 (m, 1H), 5.10-5.01 (m, 1H), 4.58 (s, 2H), 4.55-4.47 (m, 1H), 4.39 (s, 2H), 4.15-4.04 (m, 2H), 3.94-3.86 (m, 2H), 3.78 (d, J=4.0 Hz, 4H), 3.76-3.65 (m, 7H), 3.63-3.54 (m, 3H), 3.53-3.49 (m, 1H), 3.22 (s, 3H), 2.97-2.87 (m, 2H), 2.83-2.67 (m, 2H), 2.54-2.46 (m, 2H), 2.46-2.39 (m, 2H), 2.27-2.19 (m, 2H), 2.14-2.00 (m, 3H), 1.90-1.73 (m, 4H), 1.66-1.53 (m, 2H), 1.48-1.39 (m, 6H), 1.35-1.28 (m, 2H), 1.27-1.20 (m, 1H). ¹⁹F NMR (376 MHz, METHANOL-d₄) δ=−120.8.

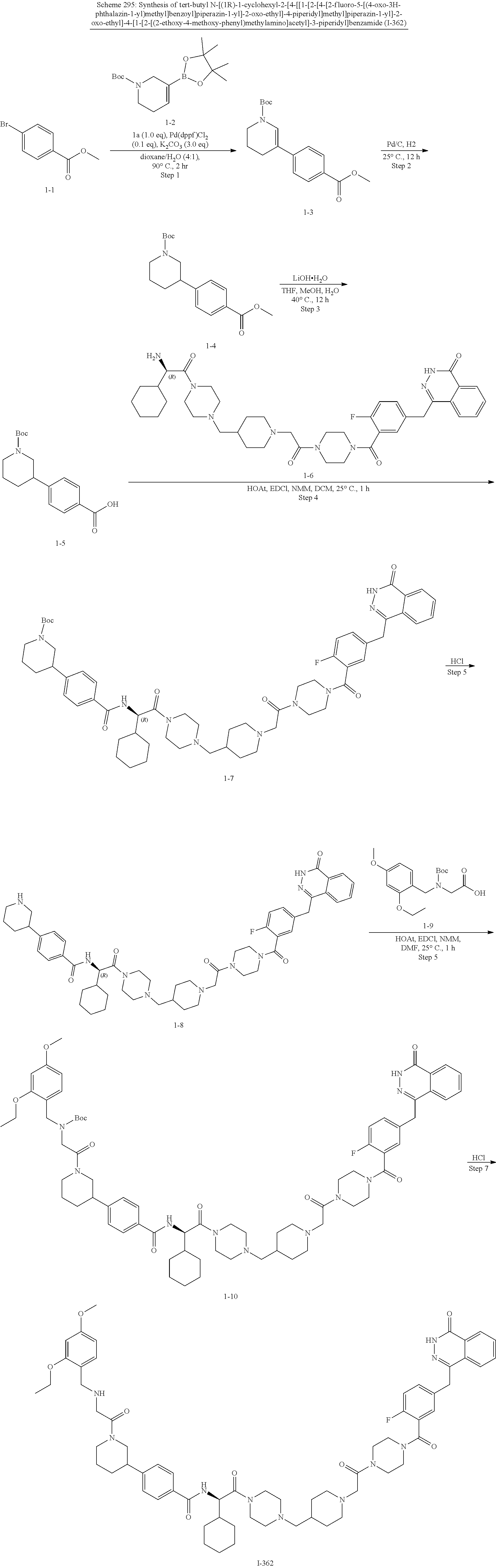

### Step 1: Synthesis of Intermediate 1-3

To a solution of Intermediate 1-1 (1.5 g, 6.98 mmol, 1 eq) and Intermediate 1-2 (2.16 g, 6.98 mmol, 1 eq) in dioxane (16 mL) and H₂O (4 mL) was added Pd(dppf)Cl₂ (510.39 mg, 697.53 μmol, 0.1 eq) and K₂CO₃ (2.89 g, 20.93 mmol, 3 eq). The suspension was degassed and purged with N₂ 3 times. The mixture was stirred at 90° C. for 1 hr under N₂. The reaction mixture was diluted with water (60 mL) and extracted with ethyl acetate (20 mL×3). The combined organic layers were washed with brine (20 mL×3) and dried over sodium sulfate, filtered and concentrated under reduced pressure to give a residue. The residue was purified by flash silica gel chromatography (ISCO; 20 g SepaFlash Silica Flash Column, Eluent of 0˜15% Ethylacetate/Petroleum ether gradient @40 mL/min). The eluent was concentrated under reduced pressure to give Intermediate 1-3 (2.1 g, 6.49 mmol, 93.04% yield, 98.0% purity) as a yellow solid. LCMS (Method H): Rt: 0.772 min, (M+H-100)⁺=218.0. ¹H NMR (400 MHz, CHLOROFORM-d) δ=8.01 (d, J=8.0 Hz, 2H), 7.44 (d, J=8.0 Hz, 2H), 6.34 (s, 1H), 4.30 (s, 2H), 3.92 (s, 3H), 3.57 (t, J=5.6 Hz, 2H), 2.35 (s, 2H), 1.51 (s, 9H).

### Step 2: Synthesis of Intermediate 1-4

To a solution of Intermediate 1-3 (2.1 g, 6.62 mmol, 1 eq) in MeOH (20 mL) was added Pd/C (400 mg, 375.87 μmol, 10%). The suspension was degassed and purged with N₂ 3 times. Then the mixture was degassed and purged with H₂ 3 times. The mixture was stirred at 25° C. for 2 hr under H₂ (15 Psi). The mixture was filtered with diatomite and washed with MeOH (10 mL*3). Then the filtrate concentrated to give Intermediate 1-4 (2 g, 6.26 mmol, 94.64% yield) as a colorless oil. LCMS (Method D): Rt: 0.496 min, (M+H-56)⁺=264.0. ¹H NMR (400 MHz, CHLOROFORM-d) δ=7.99 (d, J=7.6 Hz, 2H), 7.31 (d, J=8.0 Hz, 2H), 4.37-4.05 (m, 2H), 3.91 (s, 3H), 2.75 (d, J=9.6 Hz, 3H), 2.04 (d, J=11.2 Hz, 1H), 1.78 (d, J=11.6 Hz, 1H), 1.71-1.59 (m, 2H), 1.48 (s, 9H).

### Step 3: Synthesis of Intermediate 1-5

To a solution of Intermediate 1-4 (2 g, 6.26 mmol, 1 eq) in MeOH (5 mL), H₂O (2.5 mL) and THF (10 mL) was added LiOH·H₂O (788.31 mg, 18.79 mmol, 3 eq). The mixture was stirred at 40° C. for 1 hr. The mixture was concentrated to give a residue. The reaction mixture was diluted with water (20 mL). Then the mixture was adjusted to pH=2-3 with a saturated citric acid aqueous solution. White solid precipitated of the water. Then the white solid was dissolved into DCM (20 mL) and the organic layer was dried over sodium sulfate, filtered and concentrated under reduced pressure to give Intermediate 1-5 (1.7 g, 5.53 mmol, 88.29% yield, 99.31% purity) as a brown solid. LCMS (Method D): Rt: 0.440 min, (M−H)=250.0. ¹H NMR (400 MHz, METHANOL-d4) δ=7.97 (d, J=8.0 Hz, 2H), 7.37 (d, J=7.6 Hz, 2H), 4.10 (d, J=12.0 Hz, 2H), 2.85 (s, 2H), 2.78-2.68 (m, 1H), 2.06-1.92 (m, 1H), 1.83-1.66 (m, 2H), 1.64-1.53 (m, 1H), 1.47 (s, 9H).

### Step 4: Synthesis of Intermediate 1-7

To a solution of Intermediate 1-5 (85 mg, 278.35 μmol, 1 eq) in DCM (1 mL) was added EDCI (160.08 mg, 835.06 μmol, 3 eq), HOAt (37.89 mg, 278.35 μmol, 38.94 μL, 1 eq) and NMM (140.77 mg, 1.39 mmol, 153.01 μL, 5 eq). The mixture was stirred at 25° C. for 0.5 hr. Then Intermediate 1-6 (202.89 mg, 278.35 μmol, 1 eq) was added into the mixture and it was stirred at 25° C. for 0.5 hr. The reaction mixture was diluted with water (12 mL) and extracted with DCM (4 mL×3). The combined organic layers were washed with brine (4 mL×3) and dried over sodium sulfate, filtered and concentrated under reduced pressure to give a residue. The crude product was purified by reversed-phase HPLC (0.1% FA condition). The eluent was concentrated under reduced pressure to remove ACN and then lyophilized to give Intermediate 1-7 (200 mg, 191.54 μmol, 68.81% yield, 97.3% purity) as a yellow solid. LCMS (Method D): Rt: 0.386 min, (M+H)⁺=1016.4. ¹H NMR (400 MHz, CHLOROFORM-d) δ=10.58-9.93 (m, 1H), 8.52-8.35 (m, 1H), 7.86-7.70 (m, 5H), 7.36-7.28 (m, 4H), 7.10-6.99 (m, 2H), 5.05 (d, J=5.6 Hz, 1H), 4.29 (s, 2H), 4.25-4.02 (m, 2H), 3.83-3.53 (m, 10H), 3.36-3.20 (m, 4H), 2.99-2.86 (m, 2H), 2.81-2.69 (m, 3H), 2.45 (d, J=8.8 Hz, 3H), 2.22 (d, J=6.4 Hz, 4H), 2.07-1.98 (m, 1H), 1.81-1.58 (m, 12H), 1.48 (s, 9H), 1.33-1.06 (m, 8H). SFC: Rt: 1.264, 1.360 min. ¹⁹F NMR (400 MHz, METHAN OL-d4) δ=−117.589.

### Step 5: Synthesis of Intermediate 1-8

To a solution of Intermediate 1-7 (170 mg, 167.28 μmol, 1 eq) in DCM (2 mL) was added TFA (307.00 mg, 2.69 mmol, 200 μL, 16.10 eq). The mixture was stirred at 25° C. for 1 hr. The mixture was adjusted to pH=7˜8 with solid sodium bicarbonate. The reaction mixture was diluted with DCM (2 mL). The residue was triturated with DCM (10 mL) and filtered to afford Intermediate 1-8 (150 mg, crude) as a white solid. LCMS (Method D): Rt: 0.275 min, (M+H)⁺=916.4.

### Step 6: Synthesis of Intermediate 1-10

To a solution of Intermediate 1-8 (55 mg, 162.06 μmol, 1 eq) in DMF (1 mL) was added HOAt (22.06 mg, 162.06 μmol, 22.67 μL, 1 eq), NMM (81.96 mg, 810.30 μmol, 89.09 μL, 5 eq) and EDCI (93.20 mg, 486.18 μmol, 3 eq). The mixture was stirred at 25° C. for 0.5 hr. Then Intermediate 1-9 (148.47 mg, 162.06 μmol, 1 eq) was added into the mixture and it was stirred at 25° C. for 0.5 hr. The reaction mixture was diluted with water (12 mL) and extracted with ethyl acetate (4 mL×3). The combined organic layers were washed with brine (4 mL×3) and dried over sodium sulfate, filtered and concentrated under reduced pressure to give a residue. The crude product was purified by reversed-phase HPLC (0.1% FA condition). The eluent was concentrated under reduced pressure to remove ACN and then lyophilized to give Intermediate 1-10 (110 mg, 85.02 μmol, 52.46% yield, 95.65% purity) as a white solid. LCMS (Method D): Rt: 0.418 min, (M+H)⁺=1237.7. ¹H NMR (400 MHz, CHLOROFORM-d) δ=10.55-10.10 (m, 1H), 8.52-8.42 (m, 1H), 7.81-7.70 (m, 5H), 7.35-7.28 (m, 3H), 7.24 (d, J=8.0 Hz, 1H), 7.17-6.99 (m, 3H), 6.49-6.38 (m, 2H), 5.06-5.04 (m, 1H), 4.73-4.72 (m, 1H), 4.56-4.39 (m, 2H), 4.29 (s, 2H), 4.20-4.09 (m, 1H), 4.05-3.95 (m, 3H), 3.80 (d, J=7.6 Hz, 4H), 3.71 (s, 4H), 3.64-3.54 (m, 4H), 3.35-3.21 (m, 4H), 3.04-2.89 (m, 3H), 2.78-2.68 (m, 1H), 2.63-2.55 (m, 1H), 2.50-2.35 (m, 5H), 2.22 (d, J=6.8 Hz, 12H), 2.13-1.97 (m, 2H), 1.86-1.61 (m, 12H), 1.43-1.35 (m, 3H), 1.29-1.06 (m, 8H).

### Step 7: Synthesis of I-362

To a solution of Intermediate 1-10 (100 mg, 80.81 μmol, 1 eq) in DCM (2 mL) was added HCl/dioxane (2 M, 100 L). The mixture was stirred at 25° C. for 1 hr. The mixture was concentrated to give a residue. The crude product was purified by reversed-phase HPLC (0.1% HCl condition). The eluent was concentrated under reduced pressure to remove ACN and then lyophilized to give I-362 (11.14 mg, 9.58 μmol, 11.86% yield, 97.85% purity) as a white solid. LCMS (Method H): Rt: 0.690 min, (M+H)⁺=1137.6. ¹H NMR (400 MHz, METHANOL-d4) δ=8.37 (d, J=7.6 Hz, 1H), 7.95 (d, J=6.4 Hz, 1H), 7.91-7.75 (m, 4H), 7.49 (s, 1H), 7.38 (d, J=6.4 Hz, 3H), 7.28-7.11 (m, 2H), 6.64-6.47 (m, 2H), 4.56-4.53 (m, 1H), 4.38 (s, 2H), 4.18-4.01 (m, 4H), 3.98-3.59 (m, 14H), 3.53 (s, 3H), 3.41 (s, 1H), 3.36-3.32 (m, 3H), 3.24-3.09 (m, 1H), 3.07-2.93 (m, 2H), 2.86-2.65 (m, 2H), 2.56-2.35 (m, 4H), 2.22 (d, J=10.0 Hz, 4H), 2.03 (d, J=11.6 Hz, 1H), 1.93-1.73 (m, 8H), 1.69 (d, J=11.6 Hz, 2H), 1.65-1.54 (m, 2H), 1.46-1.42 (m, 3H), 1.35-1.18 (m, 5H), 1.17-1.02 (m, 2H). ¹⁹F NMR (400 MHz, METHAN OL-d4) δ=−120.682. SFC: Rt: 4.220, 5.420 min.

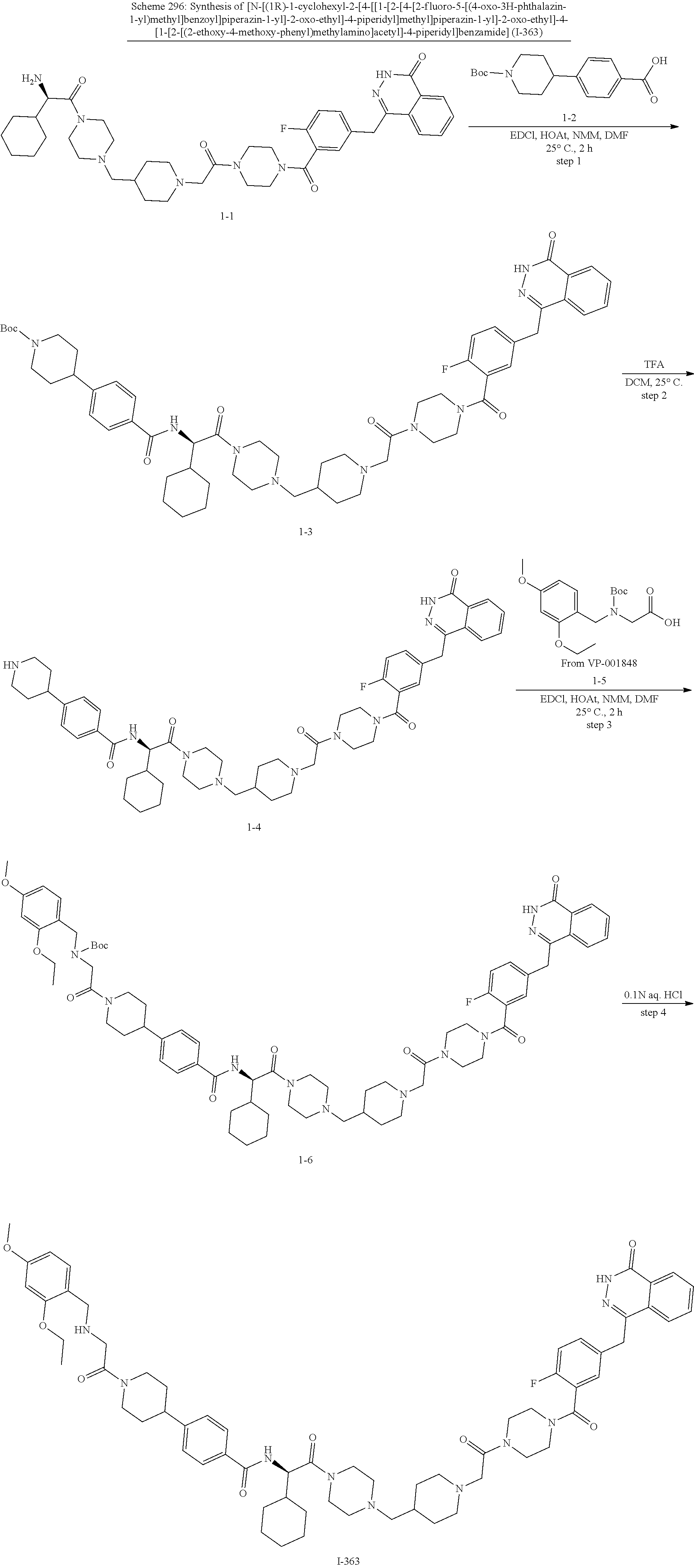

### Step 1: Synthesis of Intermediate 1-3

To a solution of intermediate 1-2 (125.68 mg, 411.58 μmol, 1.5 eq) in DMF (2 mL) was added EDCI (263.00 mg, 1.37 mmol, 5 eq), NMM (277.53 mg, 2.74 mmol, 301.67 μL, 10 eq) and HOAt (74.69 mg, 548.77 μmol, 76.77 μL, 2 eq), then intermediate 1-1 (200 mg, 274.39 μmol, 1 eq) was added in. The mixture was stirred at 25° C. for 1 hr. The residue was diluted with H₂O (5 mL) and extracted with EA (5 mL*3) dried over Na₂SO₄, filtered and concentrated under reduced pressure to give a residue. The residue was purified by flash silica gel chromatography (ISCO;12 g SepaFlash Silica Flash Column, Eluent of 0˜100% Dichloromethane/Methanol@60 mL/min). The eluent was concentrated under reduced pressure to give Intermediate 1-3 (260 mg, 255.84 μmol, 93.24% yield) as a yellow oil. LCMS (Method D): Rt=0.374 min, [M+H]⁺=1016.4.

### Step 2: Synthesis of Intermediate 1-4

A solution of intermediate 1-3 (130 mg, 127.92 μmol, 1 eq) in DCM (1 mL) and TFA (0.5 mL) was stirred at 25° C. for 1 hr. The mixture was concentrated under reduced pressure to give a residue that was then washed by PE (3*1 mL). The crude product intermediate 1-4 (120 mg, crude, TFA salt) was obtained as a yellow oil and used in the next step without further purification. LCMS (Method D): Rt=0.270 min, [M+H]⁺=916.5.

### Step 3: Synthesis of Intermediate 1-6

To a solution of intermediate 1-5 (66.68 mg, 196.48 μmol, 1.5 eq) in DMF (1.2 mL) was added EDCI (125.55 mg, 654.93 μmol, 5 eq), HOAt (35.66 mg, 261.97 μmol, 36.65 μL, 2 eq) and NMM (132.49 mg, 1.31 mmol, 144.01 μL, 10 eq) then intermediate 1-4 (120 mg, 130.99 μmol, 1 eq) was added. The mixture was stirred at 25° C. for 2 hr. The mixture was poured into 5 mL of H₂O and then extracted with EA (5 mL*3). The combined organic layers were dried over Na₂SO₄, filtered and concentrated under reduced pressure to give a residue. The residue was purified by flash silica gel chromatography (ISCO;12 g SepaFlash Silica Flash Column, Eluent of 0˜100% Dichloromethane/Methanol@60 mL/min). The eluent was concentrated under reduced pressure to give Intermediate 1-6 (150 mg, 121.21 μmol, 92.54% yield) as a yellow solid. LCMS (Method D): Rt=0.410 min, [M+H]⁺=1237.9.

### Step 4: Synthesis of I-363

A solution of intermediate 1-6 (150 mg, 121.21 μmol, 1 eq) in aq. HCl (0.1 M, 1.21 mL, 1 eq) was stirred at 100° C. for 0.5 hr. The reaction mixture was concentrated under reduced pressure to give a residue. The crude product was purified by prep-HPLC (column: Phenomenex luna C18 150*25 mm*10 um; mobile phase: [water (HCl)-ACN]; gradient: 14%-44% B over 10 min). The eluent was concentrated under lyophilization to give I-363 (15 mg, 12.08 μmol, 9.97% yield, 94.568% purity, HCl salt) as a white solid. LCMS (Method D): Rt=0.315 min, [M+H]⁺=1137.6. ¹H NMR (400 MHz, METHANOL-d4) δ=8.37 (d, J=8.0 Hz, 1H), 7.99-7.92 (m, 1H), 7.91-7.78 (m, 4H), 7.51 (br s, 1H), 7.40-7.34 (m, 3H), 7.33-7.28 (m, 1H), 7.18 (t, J=8.8 Hz, 1H), 6.63 (s, 1H), 6.59-6.56 (m, 1H), 4.84-4.75 (m, 2H), 4.71-4.61 (m, 2H), 4.39 (s, 2H), 4.36-4.27 (m, 2H), 4.24 (s, 2H), 4.20-4.14 (m, 2H), 4.08 (br s, 1H), 4.06-3.99 (m, 1H), 3.81 (s, 5H), 3.80-3.68 (m, 6H), 3.59-3.50 (m, 2H), 3.46-3.34 (m, 5H), 3.28-3.23 (m, 2H), 3.22-3.14 (m, 3H), 2.93 (br t, J=12.0 Hz, 1H), 2.84 (br t, J=12.0 Hz, 1H), 2.44-2.28 (m, 1H), 2.21 (br d, J=15.2 Hz, 2H), 1.93 (br d, J=11.2 Hz, 5H), 1.83-1.65 (m, 7H), 1.63-1.58 (m, 1H), 1.49 (t, J=6.8 Hz, 3H), 1.40-1.04 (m, 6H). ¹⁹F NMR: (377 MHz, METHANOL-d₄) δ=−120.757. SFC: Retention time: 1.456 min.

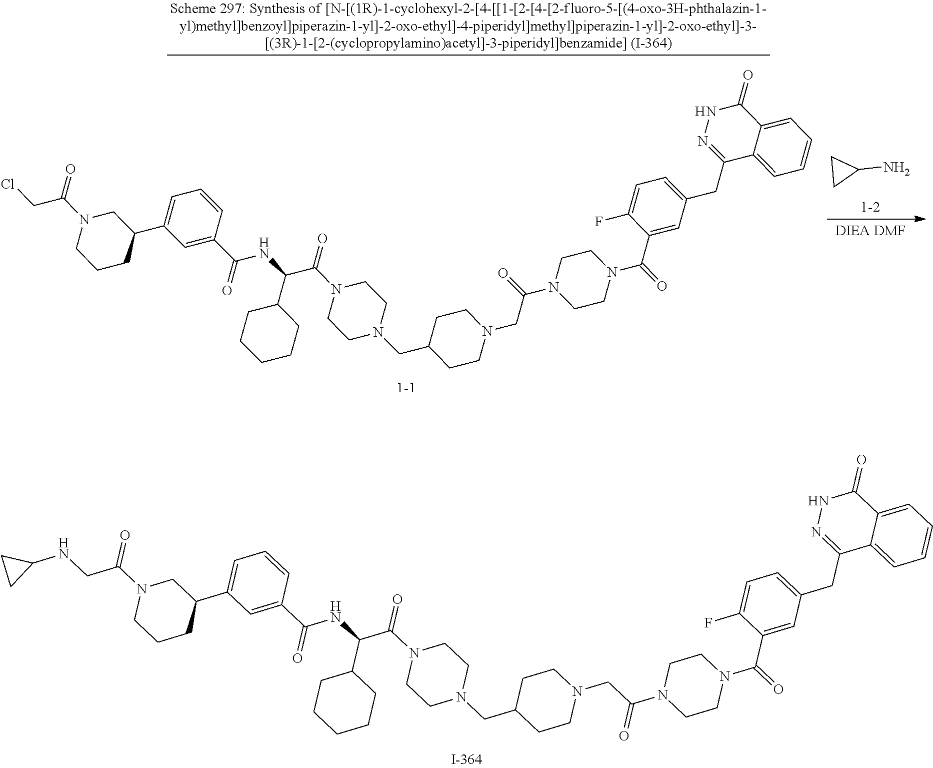

To a solution of Intermediate 1-1 (700 mg, 705.21 μmol, 1 eq) in DMF (7 mL) was added DIEA (273.43 mg, 2.12 mmol, 368.50 μL, 3 eq) and Intermediate 1-2 (60.39 mg, 1.06 mmol, 73.29 μL, 1.5 eq). The mixture was stirred at 40° C. for 12 hr. The mixture was poured into H₂O (30 mL) and stirred for 0.1 hr, and then filtered to collect the solid (500 mg, 493.46 μmol, 69.97% yield). The solid (400 mg) was purified by prep-HPLC (FA condition). The eluent was concentrated under reduced pressure to remove ACN and lyophilized to give product (244.88 mg, 231.18 μmol, 58.56% yield, FA salt) was obtained as a white solid. The white solid (100 mg) was purified by Prep-HPLC (column: Phenomenex Luna C18 150*25 mm*10 um; mobile phase: [water (FA)-ACN]; gradient: 5%-35% B over 9 min) and concentrated to remove MeCN and then lyophilized to afford I-364 (90 mg, 86.16 μmol, 87.30% yield, 97% purity) as a white solid. LCMS (Method D): Rt=0.395 min, M+H=1013.8. SFC: Rt=3.707 min. ¹H NMR (400 MHz, METHANOL-d₄) δ=8.55 (s, 1H), 8.37 (d, J=7.6 Hz, 1H), 7.98-7.91 (m, 1H), 7.90-7.75 (m, 3H), 7.74-7.68 (m, 1H), 7.52-7.35 (m, 4H), 7.18-7.14 (m, 1H), 4.95-4.89 (m, 1H), 4.61-4.52 (m, 1H), 4.38 (s, 2H), 3.92-3.49 (m, 13H), 3.37-3.32 (m, 1H), 3.25-3.10 (m, 2H), 2.98-2.68 (m, 4H), 2.54-2.33 (m, 4H), 2.28-2.01 (m, 6H), 1.97-1.50 (m, 13H), 1.44-0.97 (m, 8H), 0.54-0.35 (m, 4H).

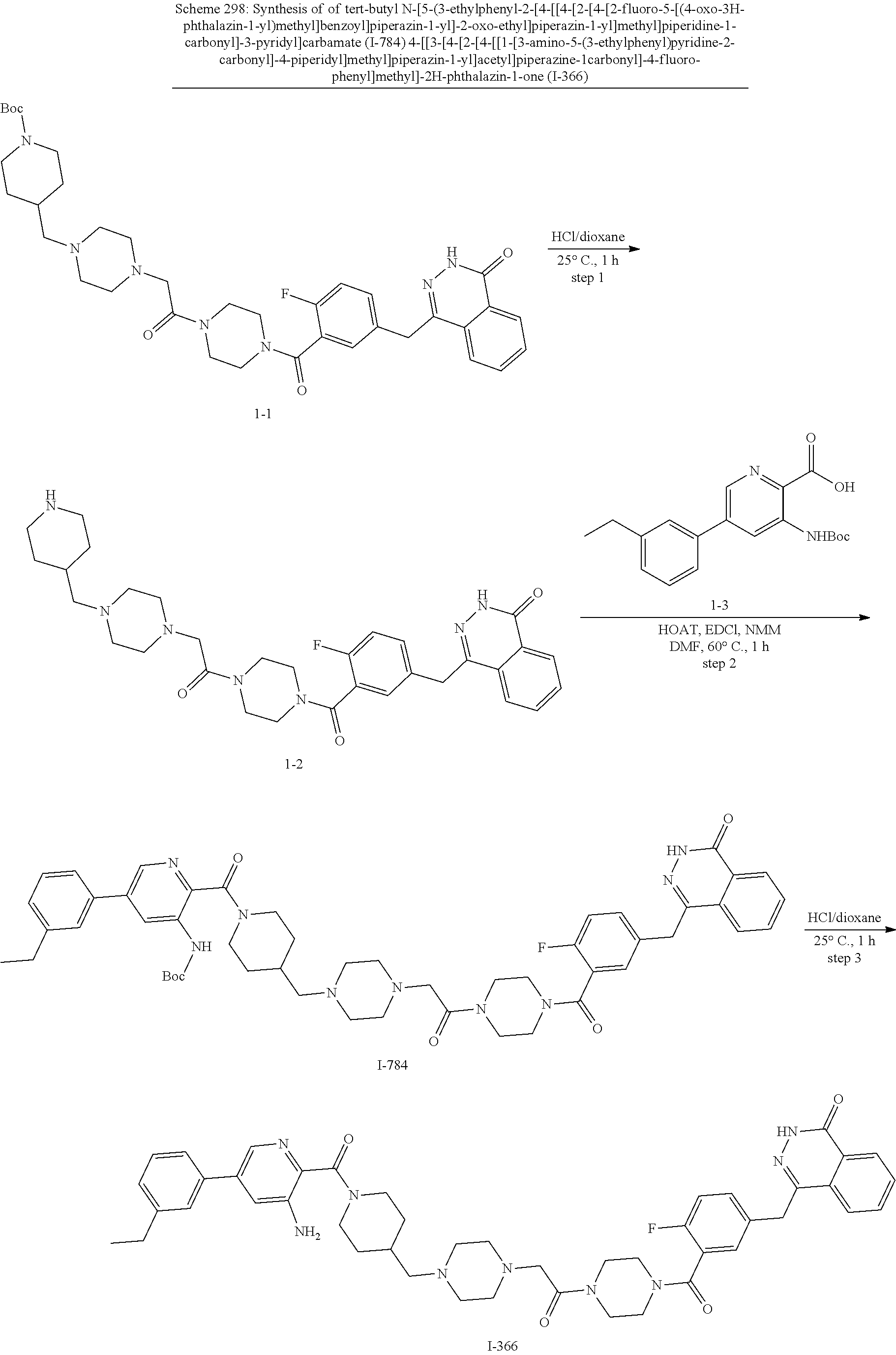 [SMILES: CC(C)(C)OC(=O)N1CCC(CN2CCN(CC(=O)N3CCN(C(=O)c4cc(Cc5n[nH]c(=O)c6ccccc56)ccc4F)CC3)CC2)CC1 ; O=C(CN1CCN(CC2CCNCC2)CC1)N1CCN(C(=O)c2cc(Cc3n[nH]c(=O)c4ccccc34)ccc2F)CC1 ; CCc1cccc(-c2cnc(C(=O)O)c(NC(=O)OC(C)(C)C)c2)c1 ; CCc1cccc(-c2cnc(C(=O)N3CCC(CN4CCN(CC(=O)N5CCN(C(=O)c6cc(Cc7n[nH]c(=O)c8ccccc78)ccc6F)CC5)CC4)CC3)c(NC(=O)OC(C)(C)C)c2)c1 ; CCc1cccc(-c2cnc(C(=O)N3CCC(CN4CCN(CC(=O)N5CCN(C(=O)c6cc(Cc7n[nH]c(=O)c8ccccc78)ccc6F)CC5)CC4)CC3)c(N)c2)c1]

### Step 1: Synthesis of Intermediate 1-2

A mixture of intermediate 1-1 (200 mg, 289.93 μmol, 1 eq) in HCl/dioxane (2 M, 2 mL, 13.80 eq) was stirred at 25° C. for 1 hr. The reaction mixture was concentrated under reduced pressure to give a residue. The residue was used in the next step without further purification. Intermediate 1-2 (300 mg, crude, HCl salt) was obtained as a yellow solid. LCMS (Method D): Rt=0.235 min, (M+H)=590.2.

### Step 2: Synthesis of I-784

To a mixture of intermediate 1-3 (131.23 mg, 383.29 μmol, 1.2 eq) and intermediate 1-2 (200 mg, 319.41 μmol, 1 eq, HCl salt) in DMF (2 mL) was added EDCI (183.69 mg, 958.22 μmol, 3 eq), HOAt (43.47 mg, 319.41 μmol, 44.68 μL, 1 eq) and NMM (161.53 mg, 1.60 mmol, 175.58 μL, 5 eq). The mixture was stirred at 60° C. for 1 hr. The reaction was poured into H₂O (2 mL) and extracted with DCM (3 mL*3). The combined organic layers were dried over Na₂SO₄, filtered and concentrated under reduced pressure to give a residue. The residue was purified by flash silica gel chromatography (ISCO; 40 g SepaFlash Silica Flash Column, Eluent of 0˜60% Ethyl acetate/Methanol gradient @40 mL/min) and concentrated under reduced pressure to give I-784 (180 mg, 184.99 μmol, 57.92% yield, 93.9% purity) as a white solid. LCMS (Method D): Rt=0.399 min, (M+H)⁺=914.4. ¹H NMR (400 MHz, METHANOL-d4) δ=8.53 (s, 1H), 8.42 (s, 1H), 8.37 (d, J=8.0 Hz, 1H), 7.94 (d, J=5.2 Hz, 1H), 7.90-7.79 (m, 2H), 7.54-7.46 (m, 3H), 7.45-7.34 (m, 2H), 7.30 (d, J=7.6 Hz, 1H), 7.16 (t, J=8.8 Hz, 1H), 4.65 (d, J=12.4 Hz, 1H), 4.38 (s, 2H), 3.82-3.65 (m, 5H), 3.53 (d, J=16.4 Hz, 2H), 3.39 (s, 1H), 3.26-3.22 (m, 1H), 3.18-3.10 (m, 1H), 2.94-2.85 (m, 1H), 2.78-2.71 (m, 2H), 2.64-2.47 (m, 7H), 2.34-2.25 (m, 2H), 1.97-1.87 (m, 2H), 1.80-1.71 (m, 1H), 1.53 (s, 10H), 1.44-1.19 (m, 7H). ¹⁹F NMR (376 MHz, METHANOL-d4) δ=−120.802.

### Step 3: Synthesis of I-366

A mixture of I-784 (50.00 mg, 54.70 μmol, 1 eq) in HCl/dioxane (2 M, 0.5 mL, 18.28 eq) was stirred at 25° C. for 1 hr. The reaction mixture was concentrated under reduced pressure to give a residue. The residue was purified by prep-HPLC (column: Phenomenex luna C18 150*25 mm*10 um; mobile phase: [water(HCl)-ACN]; gradient: 18%-48% B over 12 min) followed by lyophilization to give I-366 (21 mg, 24.69 μmol, 45.14% yield, 100% purity, HCl salt) as a white solid. LCMS (Method D): Rt=0.339 min, (M+H)⁺=814.3. ¹H NMR (400 MHz, METHANOL-d4) δ=8.37 (d, J=7.6 Hz, 1H), 8.31 (s, 1H), 7.99-7.92 (m, 2H), 7.91-7.80 (m, 2H), 7.56 (s, 1H), 7.54-7.44 (m, 3H), 7.41-7.33 (m, 2H), 7.17 (t, J=8.8 Hz, 1H), 4.39 (s, 2H), 4.22-4.15 (m, 2H), 3.87-3.68 (m, 4H), 3.54 (s, 9H), 3.46-3.37 (m, 3H), 3.35 (d, J=6.8 Hz, 1H), 3.26-3.15 (m, 2H), 3.09 (s, 2H), 2.80-2.72 (m, 2H), 2.25 (s, 1H), 2.01 (d, J=12.4 Hz, 2H), 1.55-1.22 (m, 6H). ¹⁹F NMR (376 MHz, METHAN OL-d4) δ=−120.779.

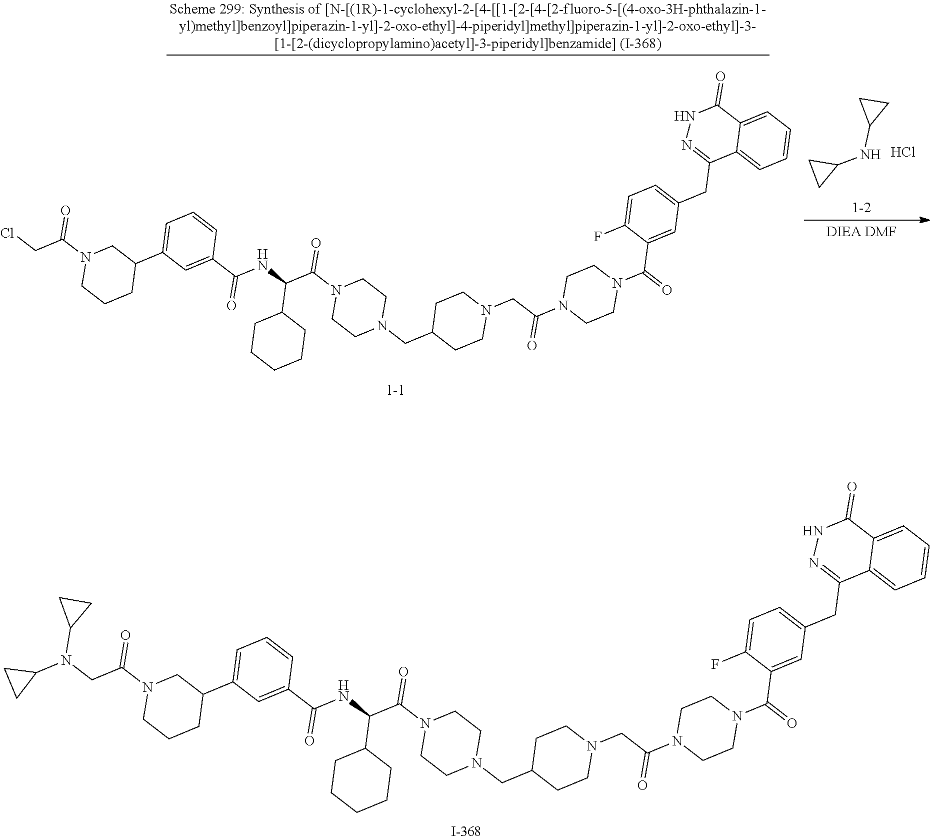

To a mixture of Intermediate 1-1 (50 mg, 50.37 μmol, 1 eq) and Intermediate 1-2 (6.73 mg, 50.37 μmol, 1 eq) in DMF (1 mL) was added DIEA (32.55 mg, 251.86 μmol, 43.87 μL, 5 eq), then the mixture was stirred at 40° C. for 1 hour. The reaction mixture was diluted with water (50 mL) and extracted with EA (10 mL*3). The combined organic layers were concentrated under reduced pressure to give a residue. The crude product was purified by reversed-phase HPLC (neutral condition). The eluent was concentrated under reduced pressure to remove MeCN and then lyophilized to give I-368 (9.28 mg, 8.35 μmol, 16.58% yield, 94.8% purity) as a white solid which. LCMS (Method D): Retention time: 0.310 min, (M+H)⁺=1053.4. SFC: Rt=2.376, Rt=2.663 Rt=2.376. ¹H NMR (400 MHz, METHANOL-d4) δ=8.40-8.35 (m, 1H), 8.00-7.92 (m, 1H), 7.90-7.81 (m, 2H), 7.78-7.67 (m, 2H), 7.53-7.41 (m, 3H), 7.37 (d, J=4.0 Hz, 1H), 7.19-7.14 (m, 1H), 4.91 (s, 1H), 4.58 (d, J=5.6 Hz, 1H), 4.39 (s, 2H), 4.13-4.05 (m, 1H), 3.85-3.65 (m, 7H), 3.60-3.48 (m, 5H), 3.21 (s, 1H), 3.09-3.01 (m, 1H), 2.96-2.85 (m, 2H), 2.78-2.63 (m, 2H), 2.53-2.36 (m, 4H), 2.27-2.18 (m, 3H), 2.15-2.02 (m, 4H), 1.94-1.65 (m, 11H), 1.64-1.54 (m, 2H), 1.39-1.04 (m, 9H), 0.49-0.31 (m, 8H). ¹⁹F NMR (376 MHz, METHANOL-d4) δ=−120.80.

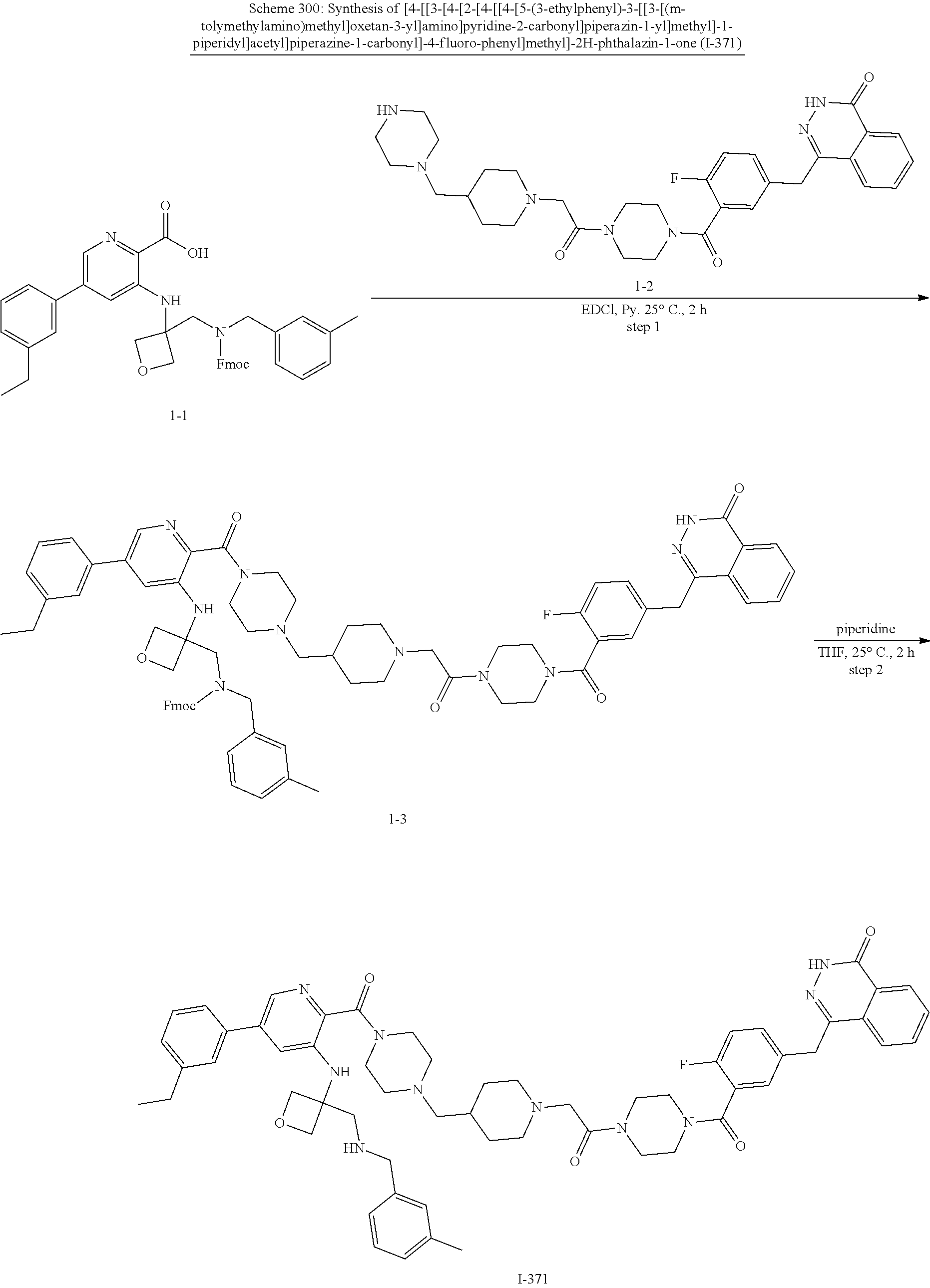

### Step 1: Synthesis of Intermediate 1-3

To a solution of intermediate 1-1 (50 mg, 76.48 μmol, 1 eq) in Py (0.5 mL) was added EDCI (73.31 mg, 382.40 μmol, 5 eq) and then intermediate 1-2 (54.12 mg, 91.78 μmol, 1.2 eq) was added. The mixture was stirred at 25° C. for 1 hr. The residue was diluted with H₂O (5 mL) and extracted with EA (5 mL*3), dried over Na₂SO₄, filtered and concentrated under reduced pressure to give a residue. The residue was purified by flash silica gel chromatography (ISCO; 12 g SepaFlash Silica Flash Column, Eluent of 0˜100% Dichloromethane/Methanol@60 mL/min). The eluent was concentrated under reduced pressure to give Intermediate 1-3 (75 mg, 61.20 μmol, 80.02% yield) as yellow oil. LCMS (Method D): Rt=0.513 min, [½M+H]⁺=613.6.

### Step 2: Synthesis of I-371

To a solution of intermediate 1-3 (70 mg, 57.12 μmol, 1 eq) in THF (0.7 mL) and piperidine (0.09 mL). The mixture was stirred at 25° C. for 2 h. The mixture was concentrated under reduced pressure to give a residue. The crude product was purified by reversed-phase HPLC (0.1% NH₃·H₂O condition). The eluent was concentrated under reduced pressure to remove ACN, then lyophilized to afford I-371 (18 mg, 17.81 μmol, 31.18% yield, 99.26% purity) as a white solid. LCMS (Method D): Rt=0.324 min, [M+H]⁺=1003.4. ¹H NMR (400 MHz, METHANOL-d4) δ=8.40-8.33 (m, 1H), 8.11 (d, J=1.6 Hz, 1H), 7.99-7.93 (m, 1H), 7.90-7.78 (m, 2H), 7.48 (br d, J=2.4 Hz, 1H), 7.42-7.33 (m, 2H), 7.32-7.22 (m, 3H), 7.16 (br t, J=9.2 Hz, 1H), 7.10-6.95 (m, 3H), 6.92 (br d, J=7.6 Hz, 1H), 6.79 (d, J=1.6 Hz, 1H), 4.77 (br s, 2H), 4.66 (br d, J=6.4 Hz, 2H), 4.58 (s, 1H), 4.39 (s, 2H), 3.87-3.76 (m, 3H), 3.76-3.66 (m, 5H), 3.58-3.47 (m, 4H), 3.17 (s, 2H), 3.14 (br d, J=1.6 Hz, 1H), 2.98-2.84 (m, 2H), 2.72-2.70 (m, 2H), 2.58-2.40 (m, 4H), 2.28-2.19 (m, 2H), 2.18-2.04 (m, 5H), 1.85-1.50 (m, 4H), 1.36-1.17 (m, 6H). ¹⁹F NMR: (377 MHz, METHANOL-d₄) δ=−120.815.

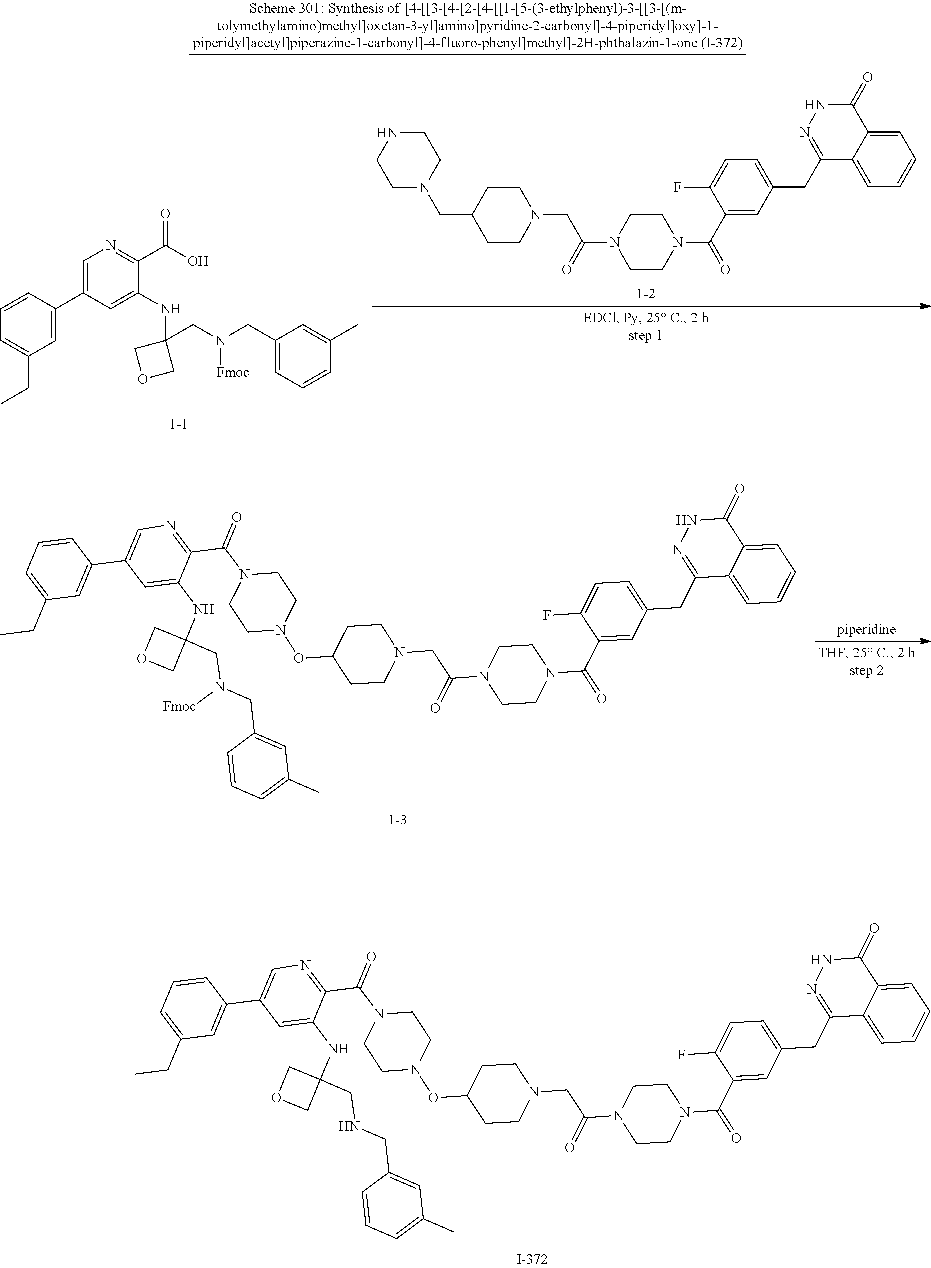

### Step 1: Synthesis of Intermediate 1-3

To a solution of intermediate 1-1 (50 mg, 76.48 μmol, 1 eq) in DMF (0.5 mL) was added HATU (43.62 mg, 114.72 μmol, 1.5 eq) and DIEA (29.65 mg, 229.44 μmol, 39.96 μL, 3 eq), then intermediate 1-2 (54.21 mg, 91.78 μmol, 1.2 eq) was added. The mixture was stirred at 25° C. for 1 hr. The residue was diluted with H₂O (5 mL) and extracted with EA (5 mL*3), dried over Na₂SO₄, filtered and concentrated under reduced pressure to give a residue. The residue was purified by flash silica gel chromatography (ISCO; 12 g SepaFlash Silica Flash Column, Eluent of 0˜100% Dichloromethane/Methanol@60 mL/min). The eluent was concentrated under reduced pressure to give Intermediate 1-3 (65 mg, 53.00 μmol, 69.30% yield) as yellow oil. LCMS (Method D): Rt=0.564 min, [M+H]⁺=1226.2.

### Step 2: Synthesis of I-372

To a solution of intermediate 1-3 (60 mg, 48.92 μmol, 1 eq) in THF (0.6 mL) was added piperidine (0.08 mL). The mixture was stirred at 25° C. for 1 hr. The mixture was concentrated under reduced pressure to give a residue. The crude product was purified by reversed-phase HPLC (0.1% NH₃·H₂O condition). The eluent was concentrated under reduced pressure to remove ACN, then lyophilized to afford I-372 (12 mg, 11.89 μmol, 24.30% yield, 99.4% purity) as a yellow solid. LCMS (Method D): Rt=0.365 min, [M+H]⁺=1004.4. ¹H NMR (400 MHz, METHANOL-d4) δ=8.40-8.31 (m, 1H), 8.10 (d, J=1.6 Hz, 1H), 7.99-7.92 (m, 1H), 7.91-7.80 (m, 2H), 7.54-7.44 (m, 1H), 7.41-7.33 (m, 2H), 7.32-7.23 (m, 3H), 7.16 (t, J=8.8 Hz, 1H), 7.09-6.96 (m, 3H), 6.96-6.88 (m, 1H), 6.81 (d, J=1.2 Hz, 1H), 4.78 (br d, J=6.8 Hz, 4H), 4.66 (br d, J=6.4 Hz, 2H), 4.61-4.54 (m, 1H), 4.39 (s, 2H), 4.13-4.00 (m, 1H), 3.83-3.64 (m, 8H), 3.59-3.46 (m, 4H), 3.26 (br s, 1H), 3.19 (br s, 1H), 3.17-3.13 (m, 2H), 2.84-2.64 (m, 4H), 2.28-2.26 (m, 2H), 2.17 (s, 3H), 1.99-1.80 (m, 4H), 1.72-1.51 (m, 4H), 1.27 (t, J=7.6 Hz, 3H). ¹⁹F NMR: (377 MHz, METHANOL-d₄) δ=−120.808.

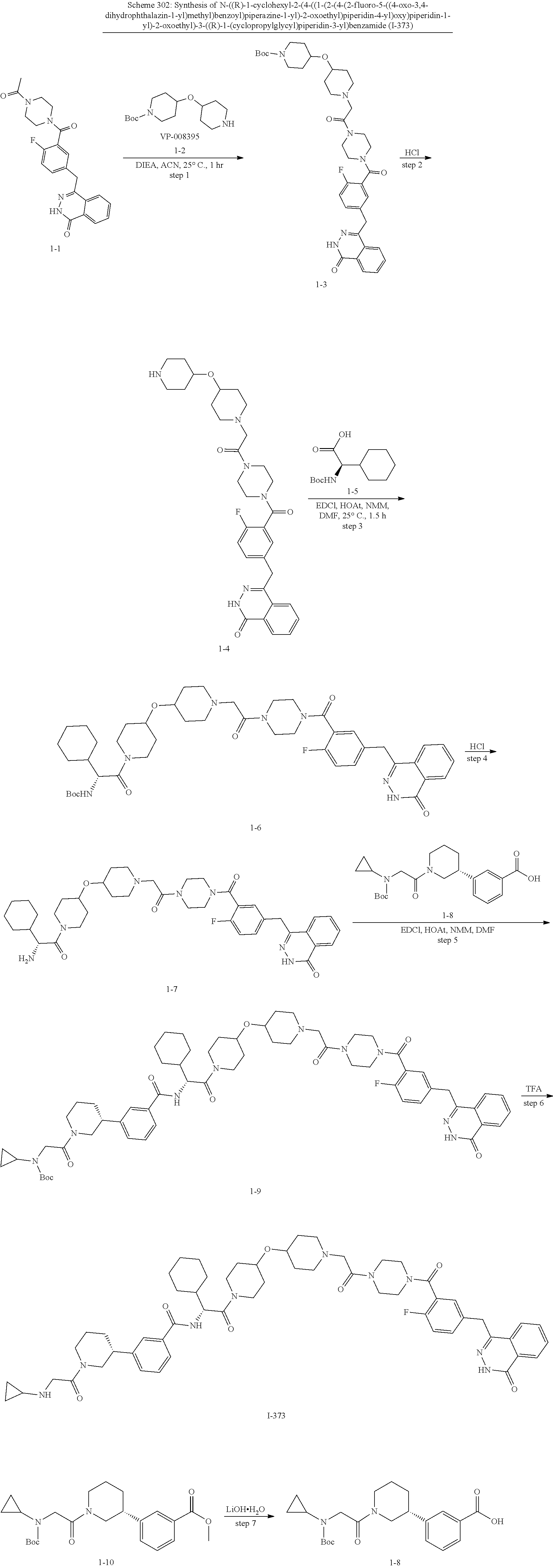

### Step 1: Synthesis of Intermediate 1-3

To a solution of Intermediate 1-1 (1.56 g, 3.52 mmol, 1 eq) and Intermediate 1-2 (1.00 g, 3.52 mmol, 1 eq) in ACN (10 mL) was added DIEA (454.45 mg, 3.52 mmol, 612.47 μL, 1 eq). The mixture was stirred at 40° C. for 1 hr. The reaction mixture was added to H₂O (15 mL) and then extracted with DCM (15 mL*3), the combined organic phase was washed, dried by Na₂SO₄, filtered and concentrated. The residue was purified by flash silica gel chromatography (ISCO; 60 g SepaFlash Silica Flash Column, Eluent of 0-100% Methanol/Dichloromethane ether gradient @60 mL/min) and the eluent was concentrated to give product. Intermediate 1-3 (2.9 g, 3.48 mmol, 99.09% yield, 83% purity) was obtained as a yellow solid. LCMS (Method D): Retention time: 0.329 min, [M+H]⁺=691.4.

### Step 2: Synthesis of Intermediate 1-4

A solution of Intermediate 1-3 (2.9 g, 3.48 mmol, 1 eq) in HCl/dioxane (2 M, 29 mL, 16.65 eq) was stirred at 25° C. for 0.5 hr. The reaction mixture was concentrated under reduced pressure to give a residue. The crude was used in the next step. Intermediate 1-4 (2.9 g, crude, HCl salt) was obtained as a white solid. LCMS (Method D): Retention time: 0.201 min, [M+H]⁺=591.3.

### Step 3: Synthesis of Intermediate 1-6

To a solution of Intermediate 1-4 (1.5 g, 2.39 mmol, 1 eq, HCl salt) and Intermediate 1-5 (615.47 mg, 2.39 mmol, 1 eq) in DMF (15 mL) was added EDCI (458.51 mg, 2.39 mmol, 1 eq) and HOAt (325.55 mg, 2.39 mmol, 334.58 μL, 1 eq) and NMM (241.92 mg, 2.39 mmol, 262.96 μL, 1 eq). The mixture was stirred at 25° C. for 1 hr. The reaction mixture was added to H₂O (15 mL) and then extracted with DCM (15 mL*3), the combined organic phase was washed, dried by Na₂SO₄, filtered and concentrated. The residue was purified by flash silica gel chromatography (ISCO; 60 g SepaFlash Silica Flash Column, Eluent of 0˜100% Ethyl acetate/Petroleum ether gradient @60 mL/min) and the eluent was concentrated to give product. Intermediate 1-6 (1.2 g, 1.45 mmol, 60.45% yield) was obtained as a white solid. LCMS (Method D): Retention time: 0.365 min, [M+H]⁺=830.5.

### Step 4: Synthesis of Intermediate 1-7

To a solution of Intermediate 1-6 (200 mg, 240.96 μmol, 1 eq) in DCM (1 mL) was added HCl/dioxane (2 M, 1 mL, 8.30 eq). The mixture was stirred at 25° C. for 0.5 hr. The reaction mixture was concentrated under reduced pressure to give a residue. The crude was used for the next step. Intermediate 1-7 (200 mg, crude, HCl) was obtained as a white solid. LCMS (Method D): Retention time: 0.270 min, [M+H]⁺=730.5.

### Step 5: Synthesis of Intermediate 1-9

To a solution of Intermediate 1-8 (70 mg, 173.92 μmol, 1 eq) and Intermediate 1-7 (133.28 mg, 173.92 μmol, 1 eq, HCl salt) in DMF (0.7 mL) was added EDCI (66.68 mg, 347.84 μmol, 2 eq) and HOAt (23.67 mg, 173.92 μmol, 24.33 μL, 1 eq) and NMM (87.96 mg, 869.60 μmol, 95.61 μL, 5 eq). The mixture was stirred at 25° C. for 1 hr. The reaction mixture was diluted with MeOH. The crude product was purified by reversed-phase HPLC (0.1% TFA condition), the eluent was concentrated to remove ACN and lyophilized to give product. Intermediate 1-9 (140 mg, 125.63 μmol, 72.24% yield) was obtained as a white solid. LCMS (Method D): Retention time: 0.420 min, [M+H]⁺=1114.6.

### Step 6: Synthesis of I-373

To a solution of Intermediate 1-9 (80 mg, 71.79 μmol, 1 eq) in DCM (0.8 mL) was added TFA (245.60 mg, 2.15 mmol, 160.00 μL, 30.00 eq). The mixture was stirred at 25° C. for 1 hr. The reaction mixture was concentrated under reduced pressure to give a residue. The crude product was purified by reversed-phase HPLC (0.1% TFA condition), the eluent was concentrated to remove ACN and lyophilized to give product. I-373 (63.42 mg, 55.57 μmol, 77.41% yield, 98.864% purity, TFA salt) was obtained as a white solid. LCMS (Method D): Retention time: 0.809 min, (M+H)=1014.6. SFC: Retention time: 6.229 min. ¹H NMR (400 MHz, METHANOL-d4) δ=8.36 (d, J=7.6 Hz, 1H), 8.32-8.24 (m, 1H), 7.99-7.91 (m, 1H), 7.90-7.67 (m, 4H), 7.58-7.40 (m, 3H), 7.40-7.31 (m, 1H), 7.17 (t, J=9.2 Hz, 1H), 4.99-4.88 (m, 2H), 4.60-4.44 (m, 1H), 4.38 (s, 2H), 4.33-4.25 (m, 2H), 4.25-4.09 (m, 2H), 4.07-3.91 (m, 2H), 3.85-3.42 (m, 11H), 3.34 (s, 3H), 3.27-3.20 (m, 1H), 3.16-2.84 (m, 2H), 2.83-2.70 (m, 2H), 2.26-1.48 (m, 18H), 1.36-1.07 (m, 6H), 0.97-0.84 (m, 4H)¹H NMR (400 MHz, METHANOL-d4) δ=8.36 (d, J=7.6 Hz, 1H), 8.32-8.24 (m, 1H), 7.99-7.91 (m, 1H), 7.90-7.67 (m, 4H), 7.58-7.40 (m, 3H), 7.40-7.31 (m, 1H), 7.17 (t, J=9.2 Hz, 1H), 4.99-4.88 (m, 2H), 4.60-4.44 (m, 1H), 4.38 (s, 2H), 4.33-4.25 (m, 2H), 4.25-4.09 (m, 2H), 4.07-3.91 (m, 2H), 3.85-3.42 (m, 11H), 3.34 (s, 3H), 3.27-3.20 (m, 1H), 3.16-2.84 (m, 2H), 2.83-2.70 (m, 2H), 2.26-1.48 (m, 18H), 1.36-1.07 (m, 6H), 0.97-0.84 (m, 4H).

### Step 7: Synthesis of Intermediate 1-8

To a solution of Intermediate 1-10 (100.00 mg, 240.09 μmol, 1 eq) in THF (0.3 mL) and MeOH (0.3 mL) and H₂O (0.3 mL) was added LiOH·H₂O (20.15 mg, 480.18 μmol, 2 eq). The mixture was stirred at 25° C. for 1 hr. The reaction mixture was adjusted to pH 7 with HCl, then the reaction mixture was concentrated under reduced pressure to give a residue. The crude was used for the next step. Intermediate 1-8 (90 mg, 223.61 μmol, 93.14% yield) was obtained as a white solid. LCMS (Method D): Retention time: 0.385 min, (M+Na)=425.2.

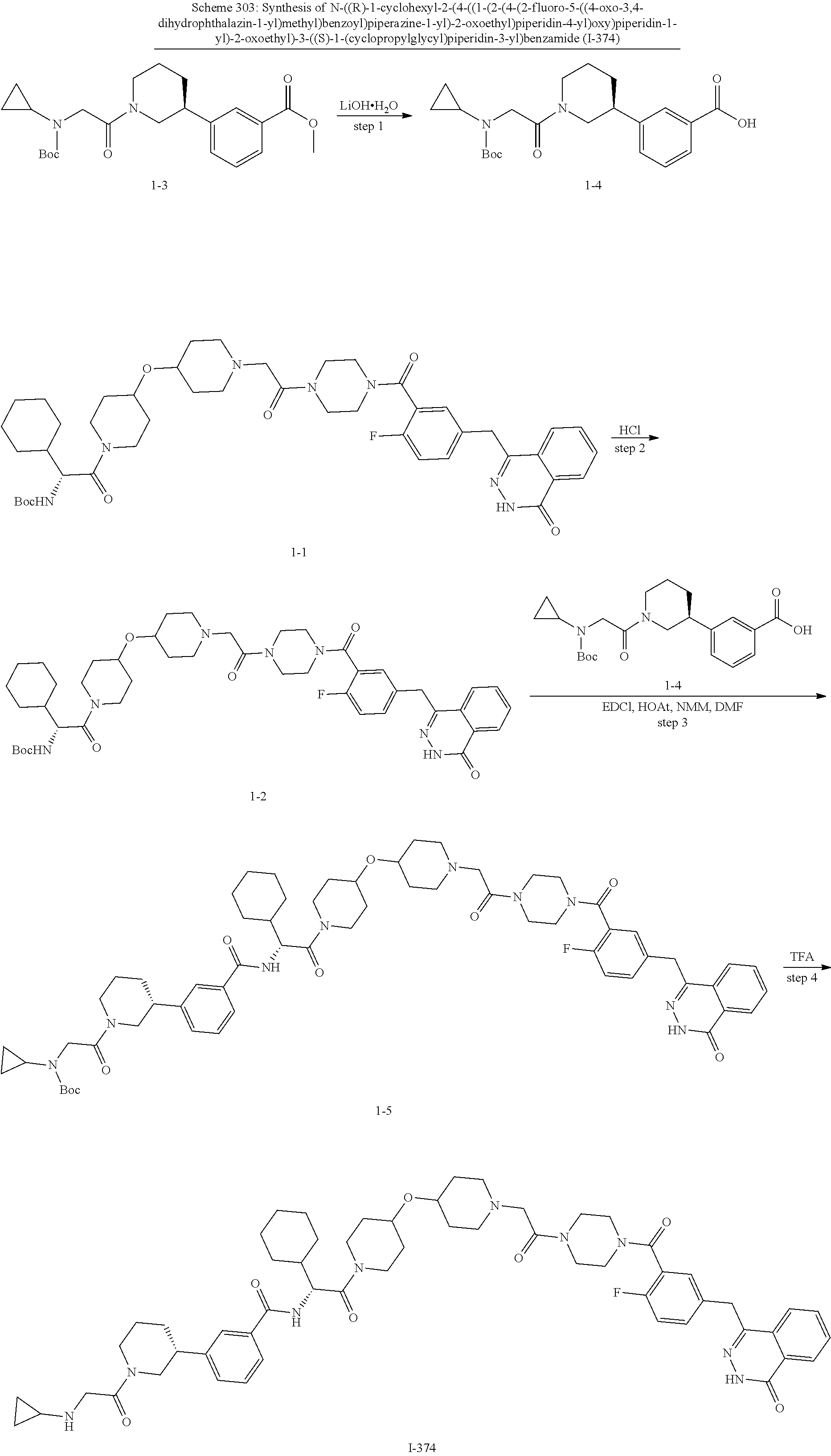

### Step 2: Synthesis of Intermediate 1-4

To a solution of Intermediate 1-3 (100.00 mg, 240.09 μmol, 1 eq) in THF (0.3 mL) and MeOH (0.3 mL) and H₂O (0.3 mL) was added LiOH·H₂O (20.15 mg, 480.18 μmol, 2 eq). The mixture was stirred at 25° C. for 1 hr. The reaction mixture was adjusted to pH 7 with HCl, then the reaction mixture was concentrated under reduced pressure to give a residue. The crude was used in the next step. Intermediate 1-4 (90 mg, 223.61 μmol, 93.14% yield) was obtained as a white solid. LCMS (Method D): Retention time: 0.380 min, [M+H]+=425.1.

### Step 1: Synthesis of Intermediate 1-2

To a solution of intermediate 1-1 (1 g, 1.20 mmol, 1 eq) in DCM (5 mL) was added HCl/dioxane (2 M, 5.00 mL, 8.30 eq). The mixture was stirred at 25° C. for 0.5 hr. The reaction mixture was concentrated under reduced pressure to give a residue. The crude was used in the next step. Intermediate 1-2 (1 g, crude, HCl salt) was obtained as a white solid. LCMS (Method D): Retention time: 0.263 min, [M+H]+=730.4. ¹H NMR (400 MHz, METHANOL-d4) δ=8.45-8.30 (m, 1H), 7.95 (d, J=6.8 Hz, 1H), 7.92-7.81 (m, 2H), 7.57-7.46 (m, 1H), 7.38-7.37 (m, 1H), 7.17 (t, J=9.2 Hz, 1H), 4.39 (s, 2H), 4.36-4.26 (m, 3H), 4.10-3.93 (m, 1H), 3.87-3.68 (m, 7H), 3.58-3.44 (m, 4H), 3.43-3.32 (m, 5H), 3.24-3.10 (m, 1H), 2.29-2.18 (m, 1H), 2.06 (d, J=1.6 Hz, 2H), 2.00-1.60 (m, 10H), 1.38-1.13 (m, 6H).

### Step 3: Synthesis of Intermediate 1-5

To a solution of Intermediate 1-2 (114.24 mg, 149.07 μmol, 1 eq, HCl salt) and Intermediate 1-4 (60 mg, 149.07 μmol, 1 eq) in DMF (0.6 mL) was added EDCI (57.16 mg, 298.15 μmol, 2 eq) and HOAt (20.29 mg, 149.07 μmol, 20.85 μL, 1 eq) and NMM (75.39 mg, 745.37 μmol, 81.95 μL, 5 eq). The mixture was stirred at 25° C. for 1 hr. The reaction mixture was diluted with MeOH. The crude product was purified by reversed-phase HPLC (0.1% TFA condition), the eluent was concentrated to remove ACN and lyophilized to give product. Intermediate 1-5 (100 mg, 89.74 μmol, 60.20% yield) was obtained as a white solid. LCMS (Method D): Retention time: 0.424 min, [M+H]+=1114.7.

### Step 4: Synthesis of I-374

To a solution of Intermediate 1-5 (120 mg, 107.69 μmol, 1 eq) in DCM (1.2 mL) was added TFA (368.40 mg, 3.23 mmol, 240.00 μL, 30.00 eq). The mixture was stirred at 25° C. for 1 hr. The reaction mixture was concentrated under reduced pressure to give a residue. The crude product was purified by reversed-phase HPLC (0.1% TFA condition), the eluent was concentrated to remove ACN and lyophilized to give product. I-374 (80.89 mg, 71.31 μmol, 66.22% yield, 99.463% purity, TFA salt) was obtained as a white solid. LCMS (Method D): Retention time: 0.339 min, [M+H]+=1014.6. SFC: Retention time: 3.934 min. ¹H NMR (400 MHz, METHANOL-d4) δ=8.37 (d, J=7.6 Hz, 1H), 8.34-8.27 (m, 1H), 8.02-7.92 (m, 1H), 7.91-7.67 (m, 4H), 7.58-7.41 (m, 3H), 7.41-7.31 (m, 1H), 7.18 (t, J=8.8 Hz, 1H), 5.00-4.90 (m, 2H), 4.62-4.52 (m, 1H), 4.39 (s, 2H), 4.33-4.26 (m, 2H), 4.25-4.10 (m, 2H), 4.08-3.92 (m, 2H), 3.85-3.43 (m, 11H), 3.35 (s, 3H), 3.25-3.00 (m, 2H), 2.93-2.84 (m, 1H), 2.84-2.71 (m, 2H), 2.26-1.50 (m, 18H), 1.37-1.20 (m, 4H), 1.19-1.05 (m, 2H), 0.98-0.81 (m, 4H).

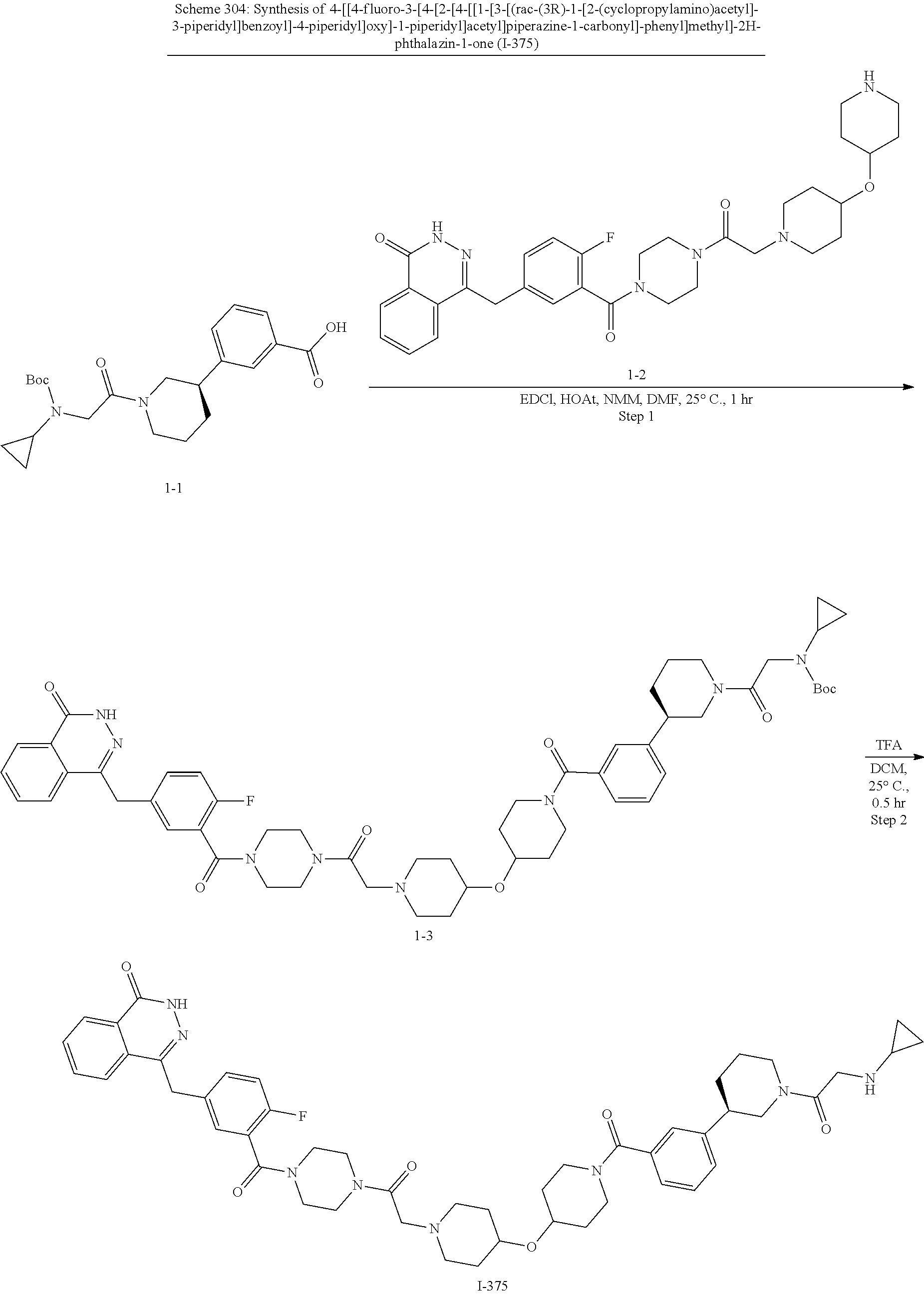 [SMILES: CC(C)(C)OC(=O)N(CC(=O)N1CCC[C@H](c2cccc(C(=O)O)c2)C1)C1CC1 ; O=C(CN1CCC(OC2CCNCC2)CC1)N1CCN(C(=O)c2cc(Cc3n[nH]c(=O)c4ccccc34)ccc2F)CC1 ; CC(C)(C)OC(=O)N(CC(=O)N1CCC[C@H](c2cccc(C(=O)N3CCC(OC4CCN(CC(=O)N5CCN(C(=O)c6cc(Cc7n[nH]c(=O)c8ccccc78)ccc6F)CC5)CC4)CC3)c2)C1)C1CC1 ; O=C(CN1CCC(OC2CCN(C(=O)c3cccc([C@H]4CCCN(C(=O)CNC5CC5)C4)c3)CC2)CC1)N1CCN(C(=O)c2cc(Cc3n[nH]c(=O)c4ccccc34)ccc2F)CC1]

### Step 1: Synthesis of Intermediate 1-3

To a solution of Intermediate 1-1 (50 mg, 124.23 μmol, 1 eq) in DMF (0.8 mL) was added EDCI (71.44 mg, 372.69 μmol, 3 eq), HOAt (16.91 mg, 124.23 μmol, 17.38 μL, 1 eq), NMM (62.83 mg, 621.14 μmol, 68.29 μL, 5 eq) and Intermediate 1-2 (85.70 mg, 136.65 μmol, 1.1 eq, HCl salt). The mixture was stirred at 25° C. for 1 hr. The mixture was filtered. The filtrate was purified by reversed-phase HPLC (0.1% FA condition). The eluent was concentrated under reduced pressure to remove ACN and then lyophilized to give Intermediate 1-3 (50 mg, 50.66 μmol, 40.78% yield, 98.8% purity) as a white solid. LCMS (Method D): Rt: 0.403 min, (M+H)⁺=975.7.

### Step 2: Synthesis of I-375

To a solution of Intermediate 1-3 (40 mg, 41.02 μmol, 1 eq) in CH₂Cl₂ (0.1 mL) was added TFA (0.5 mL) and it was stirred at 25° C. for 0.5 hr. The mixture was concentrated under vacuum. The crude product was purified by reversed-phase HPLC (0.1% TFA condition). The eluent was concentrated under reduced pressure to remove ACN and then lyophilized to give I-375 (23.75 mg, 24.01 μmol, 58.54% yield, 100% purity, TFA salt) as a white solid. LCMS (Method D): Rt: 0.299 min, (M+H)⁺=875.4. ¹H NMR (400 MHz, METHANOL-d4) δ=8.40-8.33 (m, 1H), 7.98-7.92 (m, 1H), 7.91-7.81 (m, 2H), 7.55-7.47 (m, 1H), 7.47-7.40 (m, 2H), 7.40-7.26 (m, 3H), 7.21-7.13 (m, 1H), 4.61-4.50 (m, 1H), 4.39 (s, 2H), 4.34-4.12 (m, 4H), 4.11-3.90 (m, 2H), 3.87-3.69 (m, 6H), 3.66-3.44 (m, 6H), 3.40-3.33 (m, 4H), 3.27-3.07 (m, 2H), 2.91-2.70 (m, 3H), 2.25-1.98 (m, 5H), 1.95-1.79 (m, 4H), 1.77-1.47 (m, 3H), 0.97-0.86 (m, 4H). SFC: Rt: 6.502 min. ¹⁹F NMR (376 MHz, METHANOL-d4) δ=−77.104, −120.715.

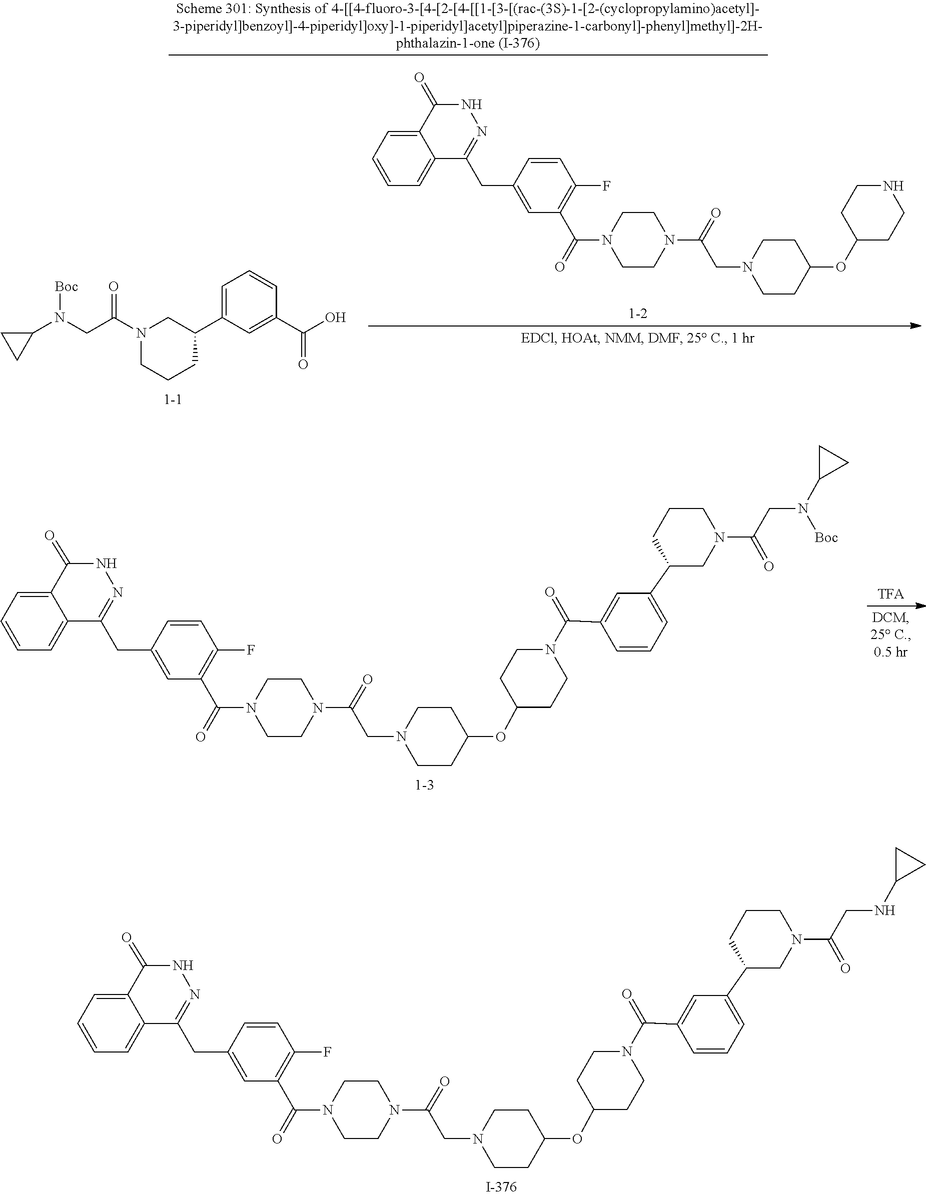 [SMILES: CC(C)(C)OC(=O)N(CC(=O)N1CCC[C@@H](c2cccc(C(=O)O)c2)C1)C1CC1 ; O=C(CN1CCC(OC2CCNCC2)CC1)N1CCN(C(=O)c2cc(Cc3n[nH]c(=O)c4ccccc34)ccc2F)CC1 ; CC(C)(C)OC(=O)N(CC(=O)N1CCC[C@@H](c2cccc(C(=O)N3CCC(OC4CCN(CC(=O)N5CCN(C(=O)c6cc(Cc7n[nH]c(=O)c8ccccc78)ccc6F)CC5)CC4)CC3)c2)C1)C1CC1 ; O=C(CN1CCC(OC2CCN(C(=O)c3cccc([C@@H]4CCCN(C(=O)CNC5CC5)C4)c3)CC2)CC1)N1CCN(C(=O)c2cc(Cc3n[nH]c(=O)c4ccccc34)ccc2F)CC1]

### Step 1: Synthesis of Intermediate 1-3

To a solution of intermediate 1-1 (80 mg, 198.77 μmol, 1 eq) in DMF (1 mL) was added EDCI (114.31 mg, 596.30 μmol, 3 eq), HOAt (27.05 mg, 198.77 μmol, 27.81 μL, 1 eq) and NMM (100.52 mg, 993.83 μmol, 109.26 μL, 5 eq). Then intermediate 1-2 (117.41 mg, 198.77 μmol, 1 eq) was added. The mixture was stirred at 25° C. for 1 hr. The resulting mixture was filtered, and the filtrate was concentrated under reduced pressure. The residue was purified by prep-HPLC (FA condition), the eluent was concentrated under reduced pressure to remove ACN and lyophilized to give Intermediate 1-3 (90 mg, 89.52 μmol, 45.04% yield, 97% purity) as a pink solid. LCMS: Rt=0.401 min, (M+H)⁺=975.6. ¹H NMR (400 MHz, METHANOL-d4) δ=8.43-8.29 (m, 2H), 7.94 (d, J=4.8 Hz, 1H), 7.90-7.80 (m, 2H), 7.54-7.46 (m, 1H), 7.45-7.39 (m, 2H), 7.39-7.30 (m, 2H), 7.28 (s, 1H), 7.17-7.14 (m, 1H), 4.53 (d, J=9.2 Hz, 1H), 4.39 (s, 2H), 4.23-4.06 (m, 2H), 4.04-3.87 (m, 3H), 3.79-3.76 (m, 5H), 3.70 (d, J=4.4 Hz, 1H), 3.63-3.49 (m, 4H), 3.48-3.33 (m, 3H), 3.28-3.08 (m, 4H), 2.98-2.85 (m, 2H), 2.82-2.65 (m, 3H), 2.09-1.93 (m, 4H), 1.91-1.78 (m, 5H), 1.72-1.53 (m, 3H), 1.46 (s, 9H), 0.74-0.61 (m, 4H). ¹⁹F NMR (376 MHz, MeOD-d4) δ=−120.762.

### Step 2: Synthesis of I-376

To a solution of intermediate 1-3 (80 mg, 82.04 μmol, 1 eq) in DCM (1 mL) was added TFA (2.69 mmol, 0.2 mL). The mixture was stirred at 25° C. for 0.5 hr. The reaction mixture was concentrated in vacuo. The residue was purified by prep-HPLC (TFA condition), the eluent was concentrated under reduced pressure to remove ACN and lyophilized to give I-376 (32.11 mg, 32.17 μmol, 39.21% yield, 99.1% purity, TFA salt) as a white solid. LCMS (Method D): Rt=0.290 min, (M+H)⁺=875.5. SFC: Rt=5.620 min, 6.540 min. ¹H NMR (400 MHz, METHANOL-d4) δ=8.37 (d, J=7.6 Hz, 1H), 7.99-7.92 (m, 1H), 7.90-7.81 (m, 2H), 7.55-7.48 (m, 1H), 7.47-7.32 (m, 4H), 7.29-7.28 (m, 1H), 7.17-7.15 (m, 1H), 4.59-4.51 (m, 1H), 4.39 (s, 2H), 4.31-4.13 (m, 4H), 4.12-3.88 (m, 2H), 3.88-3.69 (m, 6H), 3.67-3.49 (m, 5H), 3.44 (s, 1H), 3.36 (s, 3H), 3.29-3.01 (m, 3H), 2.88-2.73 (m, 3H), 2.21-2.19 (m, 1H), 2.05 (s, 4H), 1.94-1.80 (m, 4H), 1.73-1.52 (m, 3H), 0.94-0.88 (m, 4H). ¹⁹F NMR (376 MHz, MeOD-d4) δ=−77.122, −120.762.

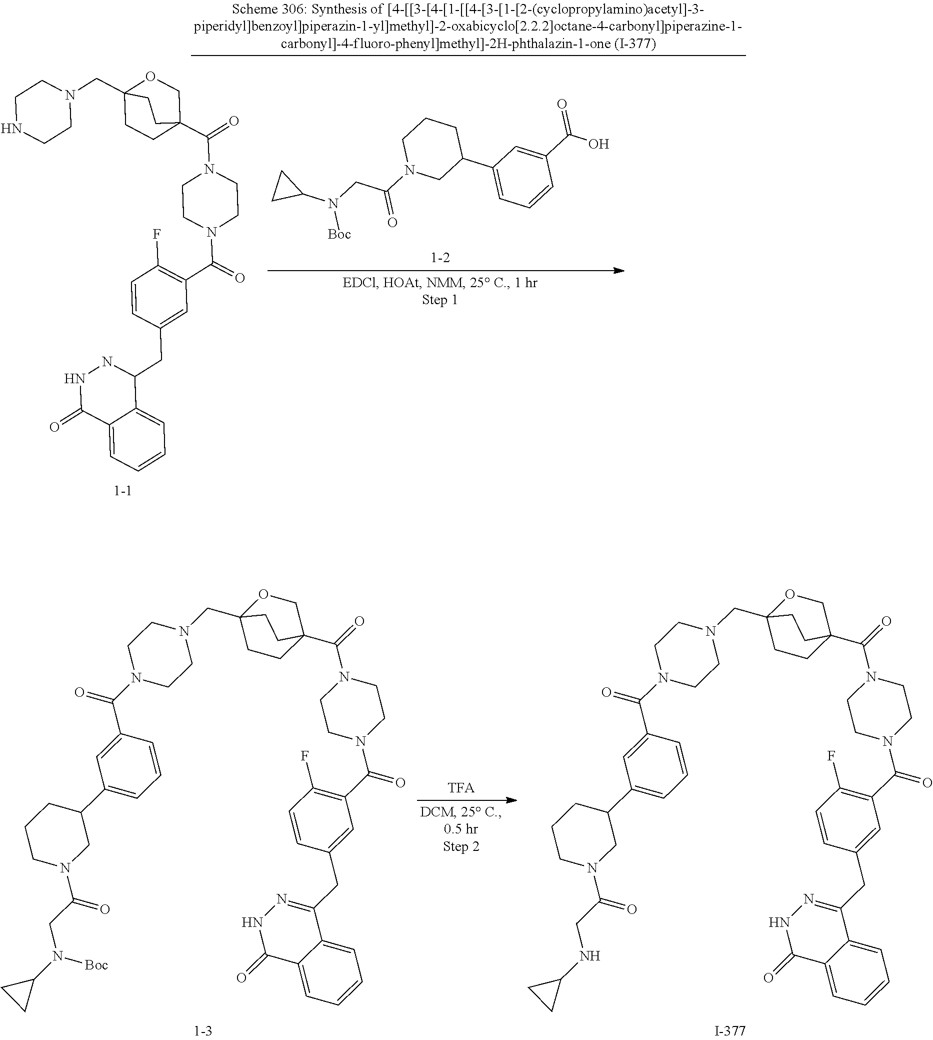

### Step 1: Synthesis of Intermediate 1-3.

To a mixture of intermediate 1-1 (152.45 mg, 238.52 μmol, 1.2 eq, HCl salt) in DMF (1 mL) was added intermediate 1-2 (80 mg, 198.77 μmol, 1 eq), EDCI (114.31 mg, 596.30 μmol, 3 eq), HOAt (27.05 mg, 198.77 μmol, 27.81 μL, 1 eq) and NMM (100.52 mg, 993.83 μmol, 109.26 μL, 5 eq) at 25° C. and the mixture was stirred at 25° C. for 1 h. The reaction mixture was concentrated to give a residue. The residue was purified by column chromatography (SiO₂, EA/MeOH=1/0 to 6/4) and concentrated to give Intermediate 1-3 (190 mg, 192.47 μmol, 96.83% yield) as a yellow gum. LCMS (Method D): Retention time: 0.373 min, (M+H)⁺=987.5.

### Step 2: Synthesis of I-377

To a mixture of intermediate 1-3 (180 mg, 182.34 μmol, 1 eq) in DCM (1 mL) was added TFA (1.54 g, 13.46 mmol, 1 mL, 73.83 eq) at 25° C., the mixture was stirred at 25° C. for 0.5 h. The reaction mixture was concentrated to give a residue. The residue was purified by reversed-phase HPLC (0.1% TFA condition) and lyophilized to give I-377 (52 mg, 50.25 μmol, 27.5% yield, 96.7% purity, TFA salt) as a white solid. LCMS (Method D): Retention time: 0.282 min, (M+H)⁺=887.4. SFC: Retention time: 2.366 min, 2.808 min. ¹H NMR (400 MHz, METHANOL-d4) δ=8.42-8.36 (m, 1H), 8.00-7.95 (m, 1H), 7.93-7.82 (m, 2H), 7.56-7.34 (m, 6H), 7.19 (t, J=9.2 Hz, 1H), 4.64-4.53 (m, 1H), 4.41 (s, 2H), 4.36-4.12 (m, 3H), 4.05 (s, 2H), 3.89-3.69 (m, 7H), 3.66-3.37 (m, 6H), 3.32-3.13 (m, 6H), 2.95-2.73 (m, 3H), 2.35-2.23 (m, 2H), 2.13-2.05 (m, 2H), 2.04-1.96 (m, 3H), 1.96-1.78 (m, 4H), 1.77-1.57 (m, 1H), 0.98-0.88 (m, 4H).

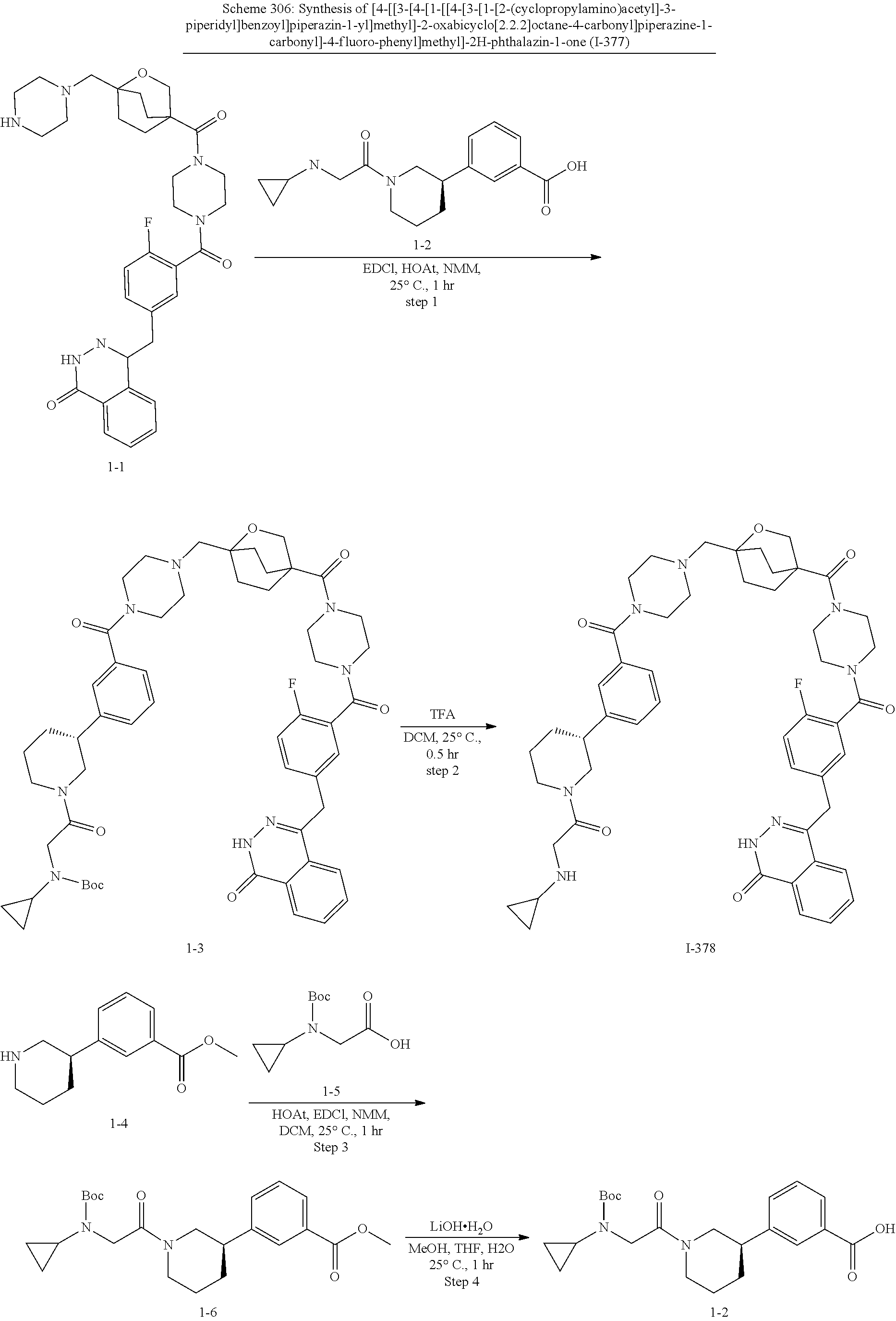

### Step 1: Synthesis of Intermediate 1-3.

To a solution of Intermediate 1-1 (152.45 mg, 238.52 μmol, 1.2 eq, HCl salt) in DMF (1 mL) was added Intermediate 1-2 (80 mg, 198.77 μmol, 1 eq), EDCI (114.31 mg, 596.30 μmol, 3 eq), HOAt (27.05 mg, 198.77 μmol, 27.81 μL, 1 eq) and NMM (100.52 mg, 993.83 μmol, 109.26 μL, 5 eq). The mixture was stirred at 25° C. for 1 hr. The mixture was concentrated under reduced pressure to give a crude product. The crude product was purified by flash silica gel chromatography (ISCO; 12 g SepaFlash Silica Flash Column, Eluent of 0˜100% Dichloromethane: Methanol ethergradient @100 mL/min, DCM/MeOH=10/1, R_{f}=0.4) and the eluent was concentrated to give Intermediate 1-3 (160 mg, 162.08 μmol, 81.54% yield, 77.24% purity) as a white solid. LCMS (Method D): Retention time: 0.396 min, (M+H)⁺=987.5.

### Step 2: Synthesis of I-378

To a solution of Intermediate 1-3 (150 mg, 151.95 μmol, 1 eq) in DCM (1.5 mL) was added TFA (2.30 g, 20.19 mmol, 1.50 mL). The mixture was stirred at 25° C. for 0.5 hr. The mixture was concentrated under reduced pressure to give a crude product. The crude product was purified by reversed-phase HPLC (0.1% TFA condition) and lyophilized to give I-378 (46.69 mg, 46.64 μmol, 30.69% yield, 100% purity, TFA salt) as a white solid. LCMS (Method D): Retention time: 0.301 min, (M+H)⁺=887.4. SFC: Retention time: 1.074 min. ¹H NMR (400 MHz, METHANOL-d₄) δ=8.40-8.34 (m, 1H), 7.98-7.92 (m, 1H), 7.91-7.81 (m, 2H), 7.53-7.34 (m, 6H), 7.17 (t, J=9.2 Hz, 1H), 4.61-4.52 (m, 1H), 4.39 (s, 2H), 4.35-4.10 (m, 3H), 4.03 (s, 2H), 3.85-3.70 (m, 7H), 3.61-3.36 (m, 6H), 3.30-3.10 (m, 6H), 2.92-2.73 (m, 3H), 2.32-2.20 (m, 2H), 2.11-2.03 (m, 2H), 2.02-1.94 (m, 3H), 1.93-1.76 (m, 4H), 1.74-1.56 (m, 1H), 0.95-0.88 (m, 4H). ¹⁹F NMR (376 MHz, METHANOL-d₄) δ=−76.979, −120.710.

### Step 3: Synthesis of Intermediate 1-6

To a solution of Intermediate 1-4 (3.6 g, 14.08 mmol, 1 eq, HCl salt) in DMF (36 mL) was added Intermediate 1-5 (3.03 g, 14.08 mmol, 1 eq), EDCI (8.10 g, 42.23 mmol, 3 eq), HOAt (1.92 g, 14.08 mmol, 1.97 mL, 1 eq) and NMM (7.12 g, 70.38 mmol, 7.74 mL, 5 eq). The mixture was stirred at 25° C. for 1 hr. The reaction mixture was quenched by addition of H₂O (100 mL), and then extracted with DCM (100 mL*3), dried over Na₂SO₄, filtered and concentrated under reduced pressure to give a residue. The residue was purified by flash silica gel chromatography (ISCO; 120 g SepaFlash Silica Flash Column, Eluent of 0˜100% Ethyl acetate/Petroleum ether gradient @80 mL/min, PE/EA=1/1, Rf=0.5) and the eluent was concentrated to give Intermediate 1-6 (4.3 g, 10.32 mmol, 73.34% yield) as a yellow solid. LCMS (Method D): Rt=0.468 min, [M+Na]⁺=439.1. SFC: Rt: 1.43 min.

### Step 4: Synthesis of Intermediate 1-2

To a solution of Intermediate 1-6 (4.3 g, 10.32 mmol, 1 eq) in H₂O (15 mL), THF (15 mL) and MeOH (15 mL) was added LiOH·H₂O (1.30 g, 30.97 mmol, 3 eq). The mixture was stirred at 25° C. for 0.5 hr. The reaction mixture was quenched by addition of H₂O (50 mL), and then extracted with DCM (50 mL), dried over Na₂SO₄, filtered and concentrated under reduced pressure to give a residue. The crudeproduct was used in the to next step. Intermediate 1-6 (2.19 g, 5.16 mmol, 49.93% yield, 94.74% purity) was obtained as a white solid. LCMS (Method D): Rt=0.381 min, [M+H]⁺=403.2. SFC: Rt: 2.07 min.

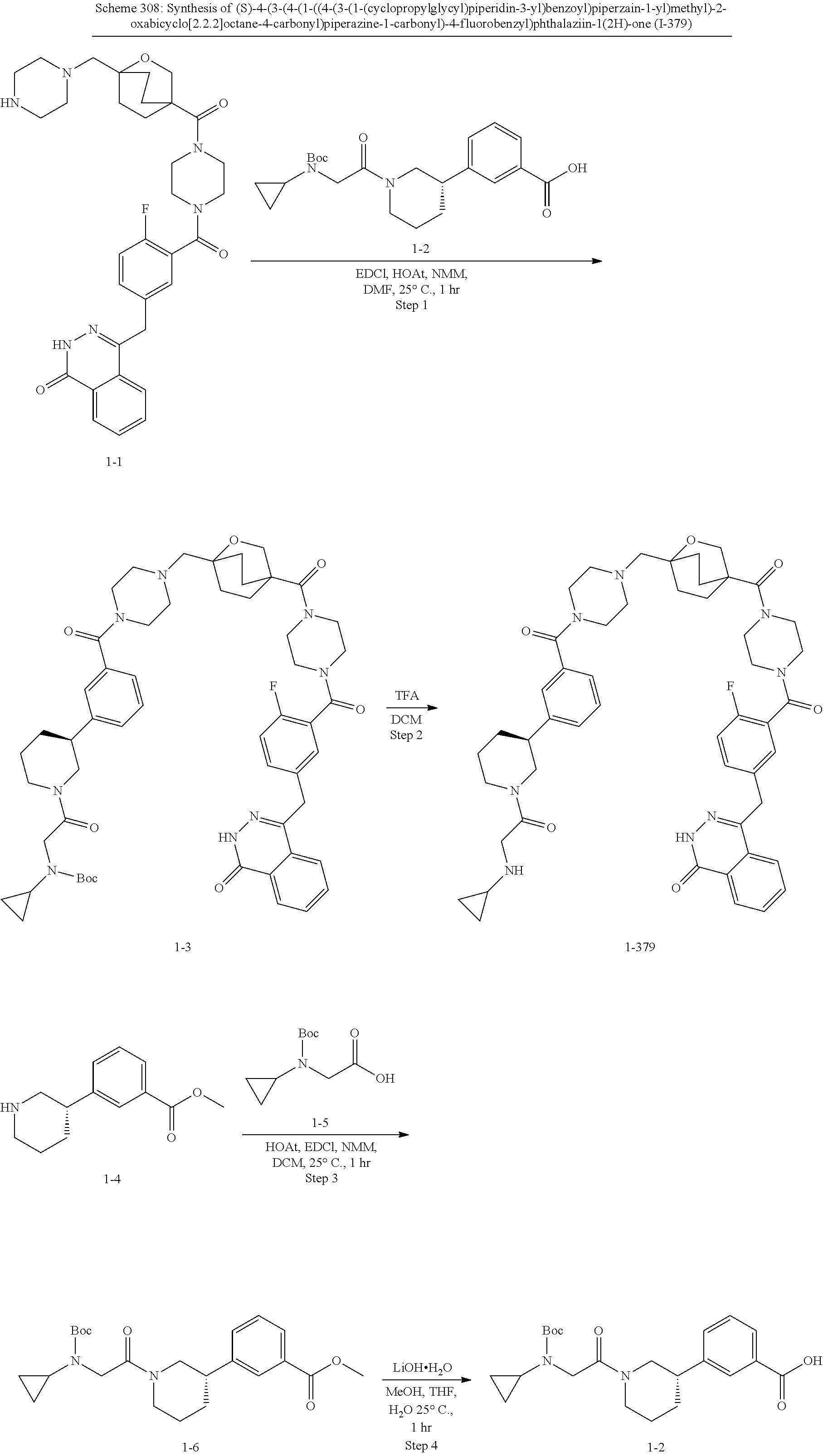

### Step 1: Synthesis of Intermediate 1-3

To a solution of intermediate 1-1 (50 mg, 78.23 μmol, 1 eq, HCl salt) and intermediate 1-2 (37.78 mg, 93.87 μmol, 1.2 eq) in DMF (0.7 mL) was added EDCI (29.99 mg, 156.46 μmol, 2 eq), NMM (39.56 mg, 391.14 μmol, 43.00 μL, 5 eq) and HOAt (5.32 mg, 39.11 μmol, 5.47 μL, 0.5 eq). The mixture was stirred at 25° C. for 1 hr. The reaction mixture was concentrated under reduced pressure to give a crude product. The crude product was purified by reversed-phase HPLC (column: Phenomenex luna C18 150*25 mm*10 um; mobile phase: [water(FA)-ACN]; gradient: 20%-500% B over 10 min) and lyophilized to give intermediate 1-3 (20 mg, 20.26 μmol, 25.90% yield) as a white solid. LCMS (Method D): Rt=0.376 min, [M/2+H]⁺=444.4.

### Step 2: Synthesis of I-379

To a solution of intermediate 1-3 (30 mg, 30.39 μmol, 1 eq) in DCM (0.15 mL) was added TFA (115.13 mg, 1.01 mmol, 75.00 μL, 33.22 eq). The mixture was stirred at 25° C. for 1 hr. The reaction mixture was concentrated to give I-379 (25.12 mg, 25.09 μmol, 82.57% yield, 100% purity, TFA salt) as a yellow solid. LCMS (Method D): Rt=0.318 min, [M+H]⁺=887.4. ¹H NMR (400 MHz, METHANOL-d4) δ=8.41-8.33 (m, 1H), 7.98-7.92 (m, 1H), 7.91-7.80 (m, 2H), 7.56-7.50 (m, 1H), 7.50-7.47 (m, 1H), 7.46 (s, 1H), 7.44-7.39 (m, 1H), 7.36 (t, J=2.4, 2H), 7.17 (t, J=9.2 Hz, 1H), 4.61-4.52 (m, 1H), 4.39 (s, 2H), 4.34-4.27 (m, 1H), 4.26 (s, 1H), 4.21-4.11 (m, 1H), 4.03 (s, 2H), 3.93-3.77 (m, 3H), 3.75 (s, 5H), 3.57 (s, 3H), 3.51-3.41 (m, 2H), 3.29-3.17 (m, 4H), 2.95-2.84 (m, 1H), 2.83-2.78 (m, 1H), 2.78-2.71 (m, 1H), 2.33-2.22 (m, 2H), 2.11-1.97 (m, 5H), 1.95 (s, 1H), 1.88-1.76 (m, 3H), 1.72 (s, 1H), 1.38-1.26 (m, 2H), 0.98-0.91 (m, 3H), 0.91-0.88 (m, 1H). F NMR (400 MHz, METHANOL-d4) δ=−77.022, −120.704.

### Step 3: Synthesis of Intermediate 1-6

To a solution of Intermediate 1-5 (4.21 g, 19.55 mmol, 1 eq) in DCM (40 mL) was added HOAt (2.66 g, 19.55 mmol, 2.73 mL, 1 eq), EDCI (11.24 g, 58.65 mmol, 3 eq), NMM (9.89 g, 97.76 mmol, 10.75 mL, 5 eq) and Intermediate 1-4 (5 g, 19.55 mmol, 1 eq, HCl salt). The mixture was stirred at 25° C. for 1 hr. The mixture was washed with water (50 mL) and extracted with DCM (20 mL*3). The combined organic layers were dried over sodium sulfate, filtered and concentrated under reduced pressure to give a residue. The residue was purified by flash silica gel chromatography (ISCO; 80 g SepaFlash Silica Flash Column, Eluent of 0˜10% EA/MeOH ether gradient @60 mL/min) and then the eluent was concentrated in vacuum to give Intermediate 1-6 (5.4 g, 12.71 mmol, 64.99% yield, 98% purity) as yellow oil. LCMS (Method D): Rt=0.540 min, [M+H]⁺=417.2. ¹H NMR (400 MHz, METHANOL-d4) δ=8.03-7.85 (m, 2H), 7.61-7.51 (m, 1H), 7.45 (m, 1H), 4.60-4.48 (m, 1H), 4.24-4.15 (m, 1H), 4.10-3.98 (m, 1H), 3.90 (d, J=2.4 Hz, 3H), 3.87 (s, 1H), 3.26-3.08 (m, 1H), 2.86-2.64 (m, 3H), 2.05 (d, J=12.8 Hz, 1H), 1.94-1.80 (m, 2H), 1.74-1.59 (m, 1H), 1.46 (s, 9H), 0.76-0.63 (m, 4H). SFC: Rt: 1.367 min, 1.445 min.

### Step 4: Synthesis of Intermediate 1-2

To a solution of Intermediate 1-6 (5.4 g, 12.96 mmol, 1 eq) in MeOH (18 mL), THF (18 mL), H₂O (18 mL) was added LiOH·H₂O (1.63 g, 38.89 mmol, 3 eq). The mixture was stirred at 25° C. for 1 hr. The pH was acidified to 3˜4 with 1N aq. HCl, and the mixture was washed with water (5 mL) and extract with EA (8 mL*3). The combined organic layers were dried over sodium sulfate, filtered and concentrated under reduced pressure to give a residue. The crude product was used in the next step without further purification. Intermediate 1-2 (5.14 g, 12.26 mmol, 94.56% yield, 96% purity) was obtained as yellow oil. LCMS (Method D): Rt=0.467 min, [M+H]⁺=403.2. ¹H NMR (400 MHz, METHANOL-d4) δ=7.98-7.93 (m, 1H), 7.92-7.87 (m, 1H), 7.57-7.49 (m, 1H), 7.43-7.42 (m, 1H), 4.54 (d, J=7.6 Hz, 1H), 4.21-4.12 (m, 1H), 4.06-3.97 (m, 1H), 3.92-3.85 (m, 1H), 3.76-3.66 (m, 1H), 3.23-3.10 (m, 1H), 2.79-2.64 (m, 3H), 1.86-1.84 (m, 3H), 1.68-1.57 (m, 1H), 1.45 (s, 9H), 0.73-0.67 (m, 2H), 0.66-0.61 (m, 2H). SFC: Rt: 1.955 min, 2.069 min.

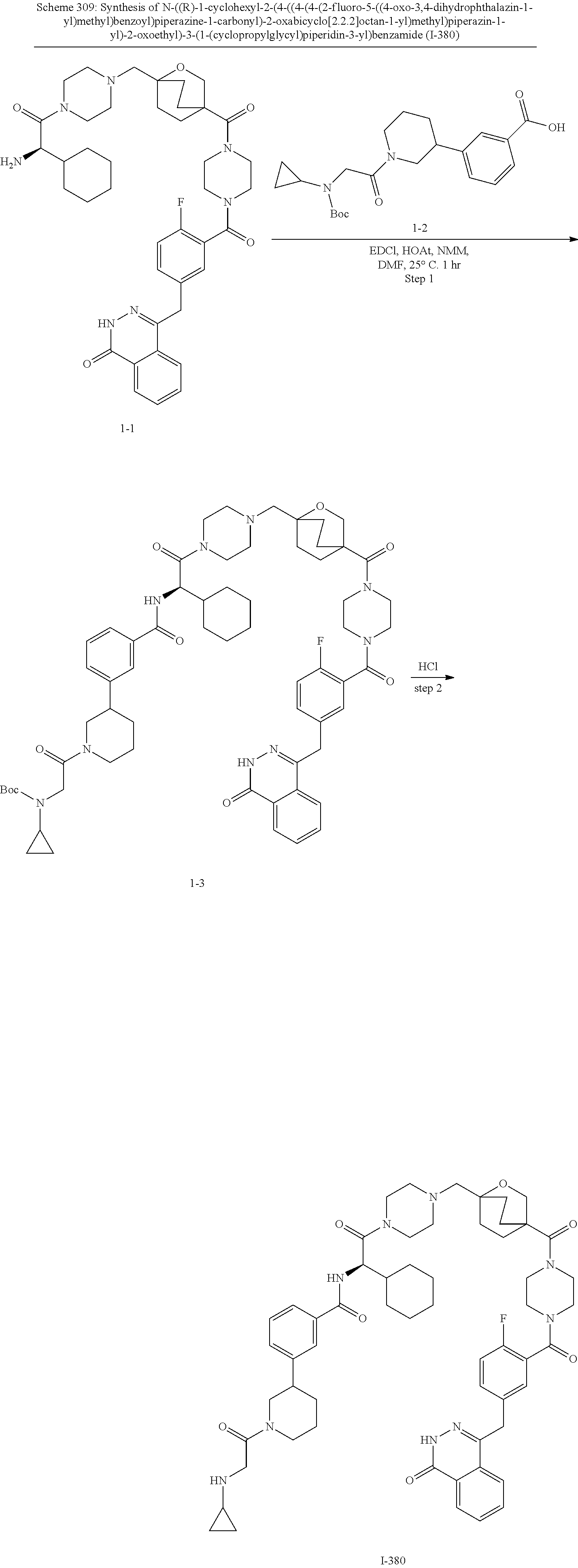

### Step 1: Synthesis of Intermediate 1-3

To a solution of Intermediate 1-1 (40 mg, 51.39 μmol, 1 eq HCl salt) and Intermediate 1-2 (20.68 mg, 51.39 μmol, 1 eq) in DMF (0.4 mL) was added EDCI (19.70 mg, 102.78 μmol, 2 eq) and NMM (25.99 mg, 256.95 μmol, 28.25 μL, 5 eq) and HOAt (6.99 mg, 51.39 μmol, 7.19 μL, 1 eq). The mixture was stirred at 25° C. for 1 hr. To the reaction mixture was added H₂O (5 mL) and then it was extracted with DCM (10 mL*3), the combined organic phase was washed, dried by Na₂SO₄, filtered and concentrated. The crude product was purified by reversed-phase HPLC (0.1% FA condition), the eluent was concentrated to remove ACN and lyophilized to give Intermediate 1-3 (45 mg, 39.95 μmol, 77.74% yield) as a white solid. LCMS (Method D): Retention time: 0.316 min, (M+H)⁺=1126.6.

### Step 2: Synthesis of I-380

To a solution of Intermediate 1-3 (30 mg, 26.63 μmol, 1 eq) in DCM (0.3 mL) was added TFA (92.10 mg, 807.73 μmol, 60.00 μL, 30.33 eq). The mixture was stirred at 25° C. for 1 hr. The reaction mixture was diluted with MeOH (1 mL). The crude product was purified by reversed-phase HPLC (0.1% TFA condition). The eluent was concentrated to remove ACN and lyophilized to give I-380 (25.54 mg, 21.82 μmol, 81.91% yield, 97.407% purity, TFA salt) as a white solid. LCMS (Method D): Retention time: 0.333 min, (M+H)⁺=1026.5. SFC: Retention time: 5.298, 6.062 min. ¹H NMR (400 MHz, METHANOL-d4) δ=8.38-8.36 (m, 1H), 7.99-7.93 (m, 1H), 7.91-7.69 (m, 4H), 7.60-7.40 (m, 3H), 7.36 (m, 1H), 7.17 (t, J=9.2 Hz, 1H), 4.84-4.76 (m, 2H), 4.69-4.43 (m, 2H), 4.39 (s, 2H), 4.33-4.11 (m, 3H), 4.05 (s, 2H), 3.76-7.34 (s, 5H), 3.67-3.38 (m, 5H), 3.29-3.11 (m, 4H), 2.96-2.85 (m, 1H), 2.84-2.74 (m, 2H), 2.41-2.20 (m, 2H), 2.18-1.86 (m, 9H), 1.86-1.48 (m, 8H), 1.42-1.04 (m, 6H), 0.96-0.86 (m, 4H).

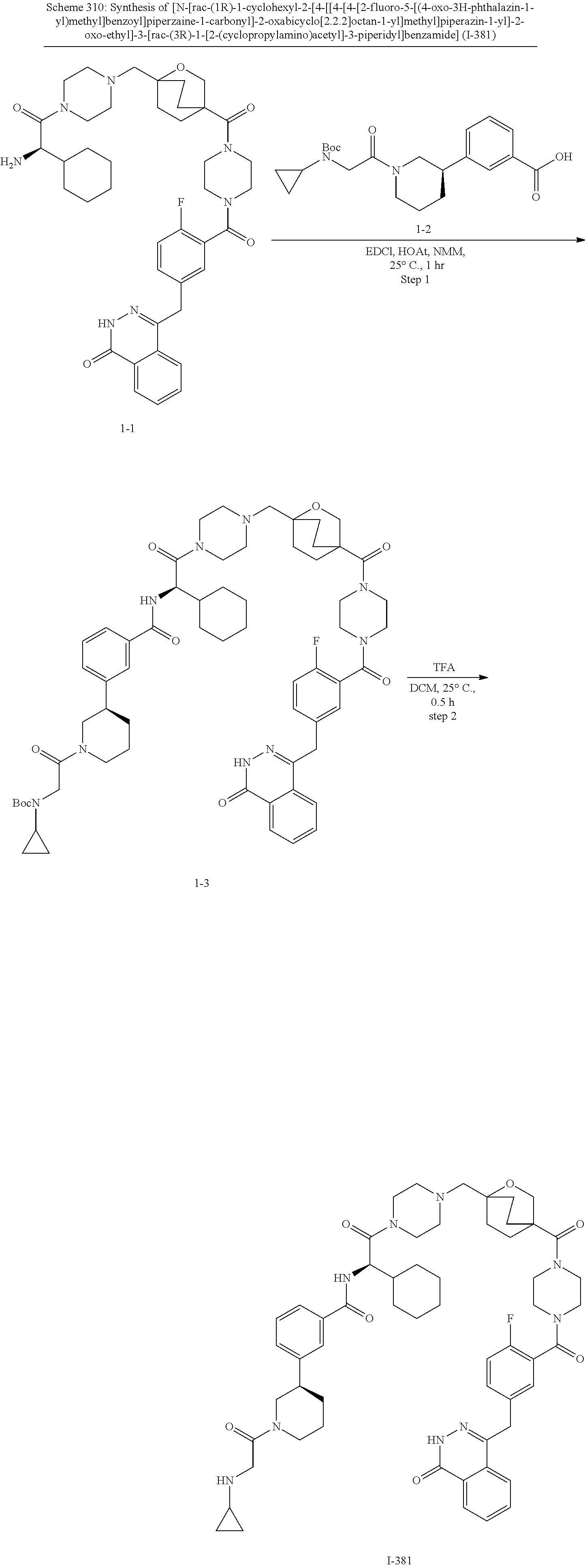

### Step 1: Synthesis of Intermediate 1-3.

To a solution of Intermediate 1-1 (70 mg, 89.93 μmol, 1 eq, HCl salt) in DMF (0.7 mL) was added Intermediate 1-2 (36.20 mg, 89.93 μmol, 1 eq), EDCI (51.72 mg, 269.80 μmol, 3 eq), HOAt (12.24 mg, 89.93 μmol, 12.58 μL, 1 eq) and NMM (45.48 mg, 449.67 μmol, 49.44 μL, 5 eq). The mixture was stirred at 25° C. for 1 hr. The mixture was concentrated under reduced pressure to give a crude product. The crude product was purified by reversed-phase HPLC (0.1% FA condition) and lyophilized to give Intermediate 1-3 (48 mg, 42.62 μmol, 47.39% yield, 100% purity) as a white solid. LCMS (Method D): Retention time: 0.479 min, (M+H)⁺=1126.6.

### Step 2: Synthesis of I-381

To a solution of Intermediate 1-3 (48 mg, 42.62 μmol, 1 eq) in DCM (0.5 mL) was added TFA (368.40 mg, 3.23 mmol, 240.00 L). The mixture was stirred at 25° C. for 0.5 hr. The mixture was concentrated under reduced pressure to give a crude product. The crude product was purified by reversed-phase HPLC (0.1% TFA condition) and lyophilized to give I-381 (15 mg, 12.73 μmol, 29.88% yield, 96.78% purity, TFA salt) as a white solid. LCMS (Method D): Retention time: 0.330 min, (M+H)⁺=1026.5. SFC: Retention time: 6.336 min. ¹H NMR (400 MHz, METHANOL-d₄) δ=8.38-7.94 (m, 1H), 7.88-7.83 (m, 1H), 7.81-7.70 (m, 2H), 7.70-7.59 (m, 2H), 7.46-7.33 (m, 3H), 7.26 (dd, J=2.0, 6.4 Hz, 1H), 7.07 (t, J=9.2 Hz, 1H), 4.53-4.38 (m, 2H), 4.29 (s, 2H), 4.21-4.12 (m, 2H), 4.11-4.02 (m, 1H), 3.97-3.92 (m, 2H), 3.77-3.57 (m, 6H), 3.51-3.27 (m, 5H), 3.18-3.07 (m, 4H), 2.94-2.54 (m, 4H), 2.23-2.11 (m, 2H), 2.04-1.48 (m, 18H), 1.27-1.00 (m, 6H), 0.84-0.78 (m, 4H). ¹⁹F NMR (376 MHz, METHANOL-d₄) δ=−77.029, −120.697.

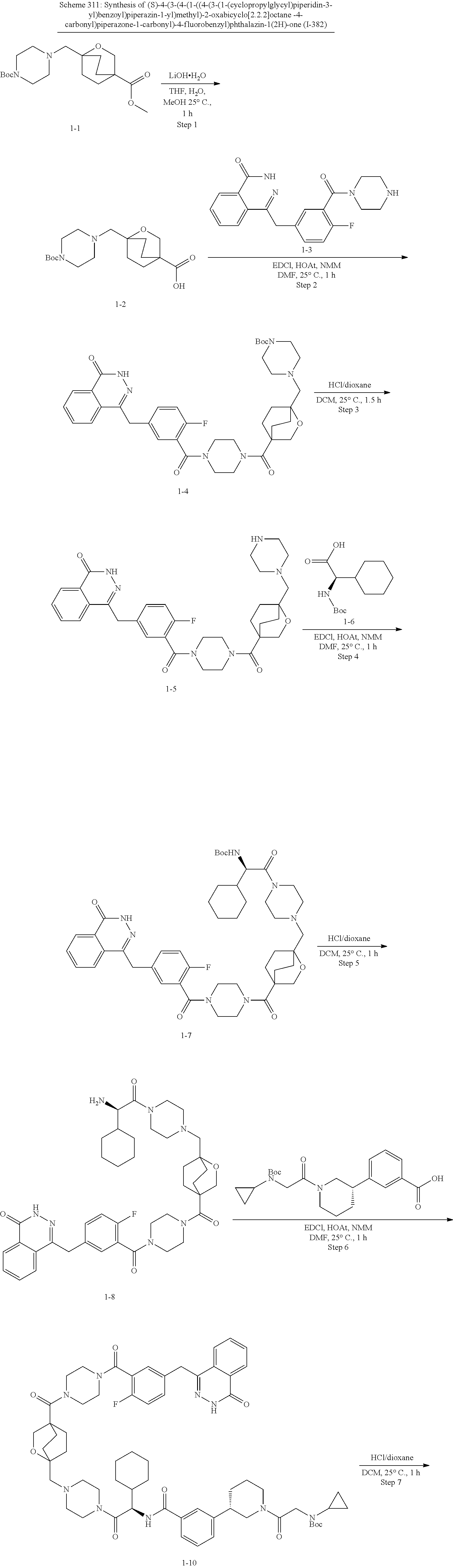

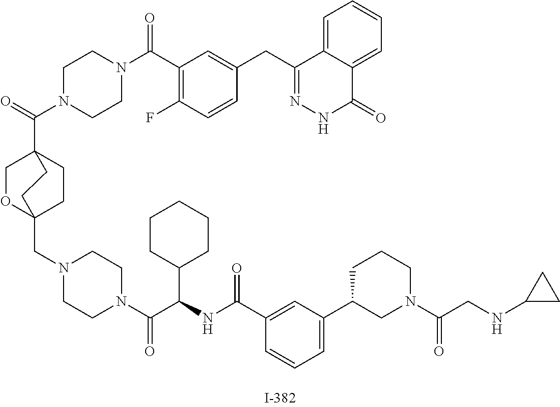 [SMILES: O=C(N[C@@H](C(=O)N1CCN(CC23CCC(C(=O)N4CCN(C(=O)c5cc(Cc6n[nH]c(=O)c7ccccc67)ccc5F)CC4)(CC2)CO3)CC1)C1CCCCC1)c1cccc([C@@H]2CCCN(C(=O)CNC3CC3)C2)c1]

### Step 1: Synthesis of Intermediate 1-2

To a solution of intermediate 1-1 (900 mg, 2.44 mmol, 1 eq) in H₂O (3 mL), THF (3 mL) and MeOH (3 mL) was added LiOH·H₂O (307.50 mg, 7.33 mmol, 3 eq). The mixture was stirred at 25° C. for 1 hr. The pH of the reaction mixture was adjusted by aq·HCl (1 M) to pH 6.0-7.0, and then the reaction mixture was quenched by adding H₂O (10 mL) at 25° C., filtered and concentrated under reduced pressure to give Intermediate 1-2 (649 mg, crude) as a white solid. LCMS (Method D): Rt=0.159 min, 0.203 min, [M+H]⁺=355.2. ¹H NMR (400 MHz, METHANOL-d4) δ=3.85 (s, 2H), 3.43 (s, 4H), 2.76 (t, J=4.8 Hz, 4H), 2.58 (s, 2H), 1.94-1.80 (m, 6H), 1.65-1.53 (m, 2H), 1.36 (s, 9H).

### Step 2: Synthesis of Intermediate 1-4

To a solution of intermediate 1-2 (629 mg, 1.77 mmol, 1 eq) and intermediate 1-3 (780.24 mg, 2.13 mmol, 1.2 eq) in DMF (6.3 mL) was added HOAt (120.77 mg, 887.32 μmol, 124.12 μL, 0.5 eq), EDCI (680.40 mg, 3.55 mmol, 2 eq) and NMM (897.49 mg, 8.87 mmol, 975.54 μL, 5 eq). The mixture was stirred at 25° C. for 1 hr. The reaction mixture was concentrated to give a crude product. The crude product was purified by reversed-phase HPLC (0.1% FA condition) and lyophilized to give Intermediate 1-4 (0.858 g, 1.21 mmol, 68.30% yield, 99.2% purity) as a yellow solid. LCMS (Method D): Rt=0.319 min, [M+H]⁺=703.4.

### Step 3: Synthesis of Intermediate 1-5

To a solution of Intermediate 1-4 (858 mg, 1.22 mmol, 1 eq) in DCM (2 mL) was added HCl/dioxane (2 M, 610.40 μL, 1 eq). The mixture was stirred at 25° C. for 1.5 hr. The reaction mixture was concentrated under reduced pressure to give Intermediate 1-5 (1 g, crude, HCl salt) as a yellow solid. LCMS (Method D): Rt=0.251 min, [M+H]⁺=603.2.

### Step 4: Synthesis of Intermediate 1-7

To a solution of intermediate 1-5 (400 mg, 625.82 μmol, 1 eq, HCl salt) and intermediate 1-6 (193.25 mg, 750.99 μmol, 1.2 eq) in DMF (4 mL) was added EDCI (239.94 mg, 1.25 mmol, 2 eq), NMM (316.50 mg, 3.13 mmol, 344.02 μL, 5 eq) and HOAt (42.59 mg, 312.91 μmol, 43.77 μL, 0.5 eq). The mixture was stirred at 25° C. for 1 hr. The reaction mixture was concentrated under reduced pressure to give a crude product. The crude product was purified by reversed-phase HPLC (0.1% FA condition) and lyophilized to give Intermediate 1-7 (0.284 g, 325.93 μmol, 52.08% yield, 96.63% purity) as a white solid. LCMS (Method D): Rt=0.364 min, [M+H]⁺=842.4.

### Step 5: Synthesis of Intermediate 1-8

To a solution of intermediate 1-7 (264 mg, 313.54 μmol, 1 eq) in DCM (0.7 mL) was added HCl/dioxane (2 M, 156.77 μL, 1 eq). The mixture was stirred at 25° C. for 1 hr. The reaction mixture was concentrated under reduced pressure to give Intermediate 1-8 (200 mg, 256.95 μmol, 81.95% yield, HCl salt) as a yellow solid. LCMS (Method D): Rt=0.313 min, [M+H]⁺=742.4.

### Step 6: Synthesis of Intermediate 1-10

To a solution of intermediate 1-8 (60 mg, 80.87 μmol, 1 eq) and intermediate 1-9 in DMF (0.6 mL) was added HOAt (5.50 mg, 40.44 μmol, 5.66 μL, 0.5 eq), NMM (40.90 mg, 404.37 μmol, 44.46 μL, 5 eq), and EDCI (31.01 mg, 161.75 μmol, 2 eq). The mixture was stirred at 25° C. for 1 hr. The reaction mixture was concentrated under reduced pressure to give a crude product. The crude product was purified by reversed-phase HPLC (0.1% FA condition) and lyophilized to give Intermediate 1-10 (42 mg, 37.29 μmol, 46.11% yield) as a white solid. LCMS (Method D): Rt=0.420 min, [M+H]⁺=1126.6.

### Step 7: Synthesis of I-382

To a solution of intermediate 1-10 (40 mg, 35.51 μmol, 1 eq) in DCM (0.1 mL) was added HCl/dioxane (2 M, 17.76 μL, 1 eq). The mixture was stirred at 25° C. for 1 hr. The reaction mixture was concentrated under reduced pressure to give the crude product. The crude product was purified by reversed-phase HPLC (column: CD01-Phenomenex luna C18 150*25*10 um; mobile phase: [water (FA)-ACN]; gradient: 12%-42% B over 10 min), the eluent was lyophilized to give I-382 (14 mg, 13.47 μmol, 37.92% yield, 98.71% purity) as a white solid. LCMS (Method D): Rt=0.377 min, [M+H]⁺=1026.6. ¹H NMR (400 MHz, METHANOL-d4) δ=8.34 (s, 1H), 7.98-7.92 (m, 1H), 7.91-7.80 (m, 2H), 7.77 (s, 1H), 7.74-7.68 (m, 1H), 7.53-7.49 (m, 1H), 7.49-7.40 (m, 2H), 7.3-7.37 (m, 1H), 7.16 (t, J=8.8 Hz, 1H), 4.90 (d, J=8.4 Hz, 2H), 4.62-4.51 (m, 1H), 4.39 (s, 2H), 3.95 (s, 2H), 3.88 (d, J=12.8 Hz, 1H), 3.75 (s, 5H), 3.73-3.61 (m, 4H), 3.61-3.53 (m, 3H), 3.53-3.35 (m, 1H), 3.25-3.12 (m, 1H), 2.94-2.82 (m, 1H), 2.82-2.72 (m, 1H), 2.67-2.45 (m, 4H), 2.38-2.27 (m, 3H), 2.25-2.14 (m, 2H), 2.11-2.02 (m, 1H), 2.01-1.83 (m, 8H), 1.81-1.74 (m, 2H), 1.73-1.55 (m, 5H), 1.36-1.19 (m, 3H), 1.17-1.04 (m, 2H), 0.59-0.42 (m, 4H). F NMR (400 MHz, METHANOL-d4) δ=−120.727.

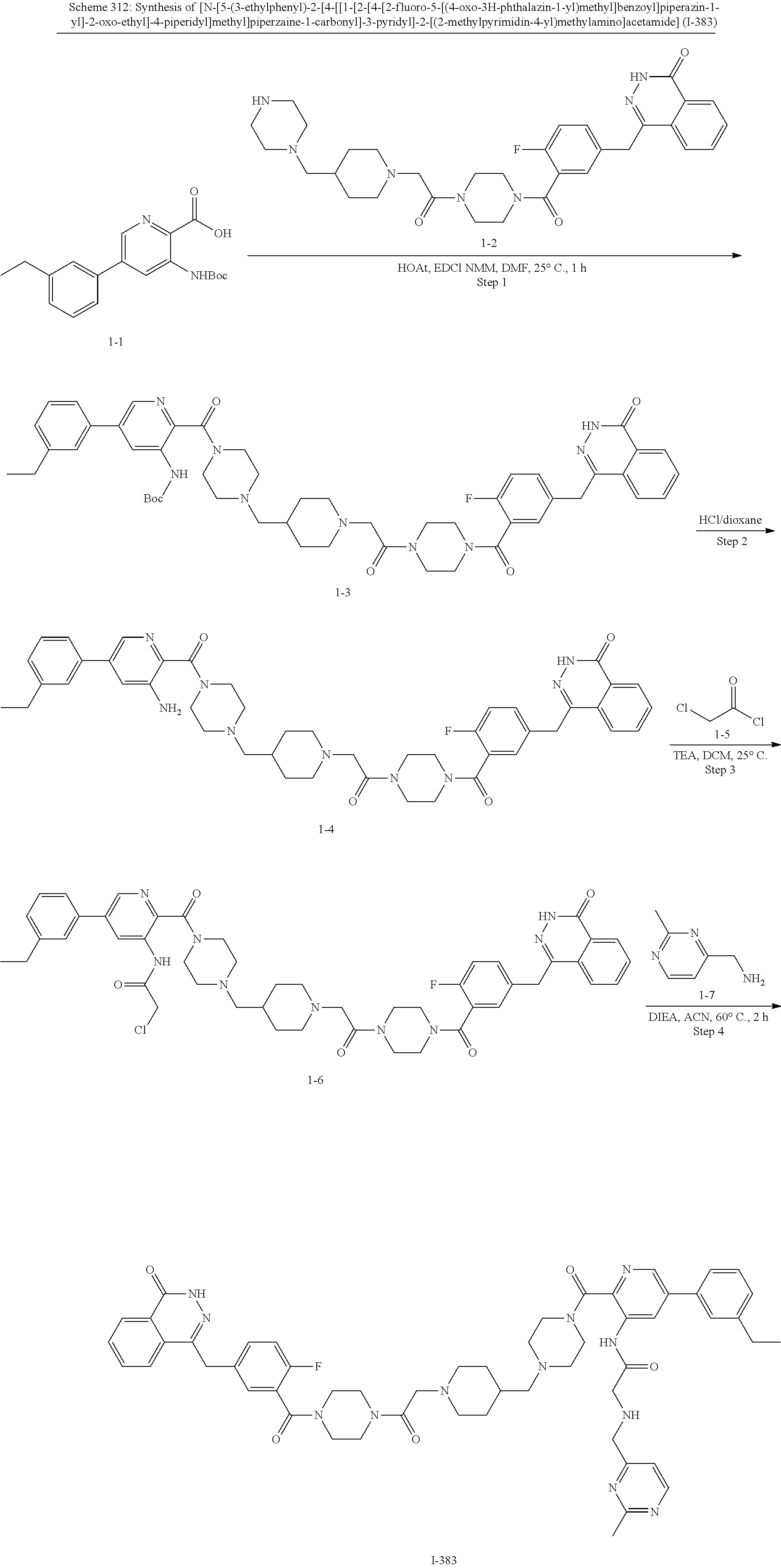

### Step 1: Synthesis of Intermediate 1-3.

To a mixture of intermediate 1-1 (1.09 g, 3.19 mmol, 1 eq) in DMF (20 mL) was added intermediate 1-2 (2 g, 3.19 mmol, 1 eq, HCl salt), HOAt (434.75 mg, 3.19 mmol, 446.81 μL, 1 eq), EDCI (1.84 g, 9.58 mmol, 3 eq) and NMM (1.62 g, 15.97 mmol, 1.76 mL, 5 eq) at 25° C., and the mixture was stirred at 25° C. for 1 h. The reaction mixture was diluted with H₂O (30 mL), extracted with EtOAc (15 mL×3), the combined organic layers were dried over Na₂SO₄, filtered and concentrated to give a residue. The residue was purified by column chromatography (SiO₂, EA/MeOH=1/0 to 7/3) and concentrated to give Intermediate 1-3 (1.31 g, 1.43 mmol, 44.87% yield) as a yellow solid. LCMS (Method D): Retention time: 0.349 min, (M+H)⁺=914.4.

### Step 2: Synthesis of Intermediate 1-4.

To a mixture of intermediate 1-3 (1.1 g, 1.20 mmol, 1 eq) in DCM (5 mL) was added HCl/dioxane (2 M, 5 mL, 8.31 eq) at 25° C., and the mixture was stirred at 25° C. for 0.5 h. The reaction mixture was concentrated to give the crude product. The crude product was used for the next step directly. Intermediate 1-4 (1 g, 1.18 mmol, 97.71% yield, HCl salt) was obtained as a yellow solid. LCMS (Method D): Retention time: 0.305 min, (M+H)⁺=814.4.

### Step 3: Synthesis of Intermediate 1-6.

To a mixture of intermediate 1-4 (1 g, 1.18 mmol, 1 eq, HCl salt) in DCM (10 mL) was added intermediate 1-5 (199.21 mg, 1.76 mmol, 140.49 μL, 1.5 eq) and TEA (356.96 mg, 3.53 mmol, 491.01 μL, 3 eq) at 0° C., the mixture was stirred at 25° C. for 0.5 h. The reaction mixture was diluted with H₂O (15 mL) and extracted with EtOAc (10 mL×3), the combined organic layers were dried over Na₂SO₄, filtered and concentrated to give a residue. The residue was purified by column chromatography (SiO₂, EA/MeOH=1/0 to 1/1) and concentrated to give Intermediate 1-6 (230 mg, 258.30 μmol, 21.97% yield) as a yellow solid. LCMS (Method D): Retention time: 0.310 min, (M+H)⁺=890.4.

### Step 4: Synthesis of I-383

To a mixture of intermediate 1-6 (110 mg, 123.53 μmol, 1 eq) in DMF (1 mL) was added intermediate 1-7 (30.43 mg, 247.07 μmol, 2 eq) and DIEA (47.90 mg, 370.60 μmol, 64.55 μL, 3 eq) at 25° C., the mixture was stirred at 60° C. for 1 h. The reaction mixture was concentrated to give a residue. The residue was purified by prep-HPLC (column: CD04-Welch Utimate C18 150*25*7 um; mobile phase: [water (TFA)-ACN]; gradient: 13%-43% B over 10 min) and lyophilized to give I-383 (13.12 mg, 11.89 μmol, 9.63% yield, 98.927% purity, TFA salt) as a yellow solid. LCMS (Method D): Retention time: 0.288 min, (M+H)⁺=977.5. ¹H NMR (400 MHz, METHANOL-d4) δ=8.77-8.67 (m, 2H), 8.40-8.33 (m, 1H), 8.25 (d, J=1.6 Hz, 1H), 7.99-7.92 (m, 1H), 7.91-7.81 (m, 2H), 7.55-7.49 (m, 3H), 7.45 (t, J=7.6 Hz, 1H), 7.42-7.31 (m, 3H), 7.18 (t, J=9.2 Hz, 1H), 4.53 (s, 2H), 4.39 (s, 2H), 4.31 (s, 1H), 4.26-4.18 (m, 3H), 3.98-3.88 (m, 2H), 3.84-3.66 (m, 5H), 3.60-3.40 (m, 7H), 3.35 (d, J=6.8 Hz, 3H), 3.24-3.06 (m, 4H), 3.01 (s, 1H), 2.81-2.69 (m, 5H), 2.36-2.24 (m, 1H), 2.22-2.08 (m, 2H), 1.82-1.63 (m, 2H), 1.30 (t, J=7.6 Hz, 3H). F NMR (376 MHz, METHANOL-d4) δ=−120.7.

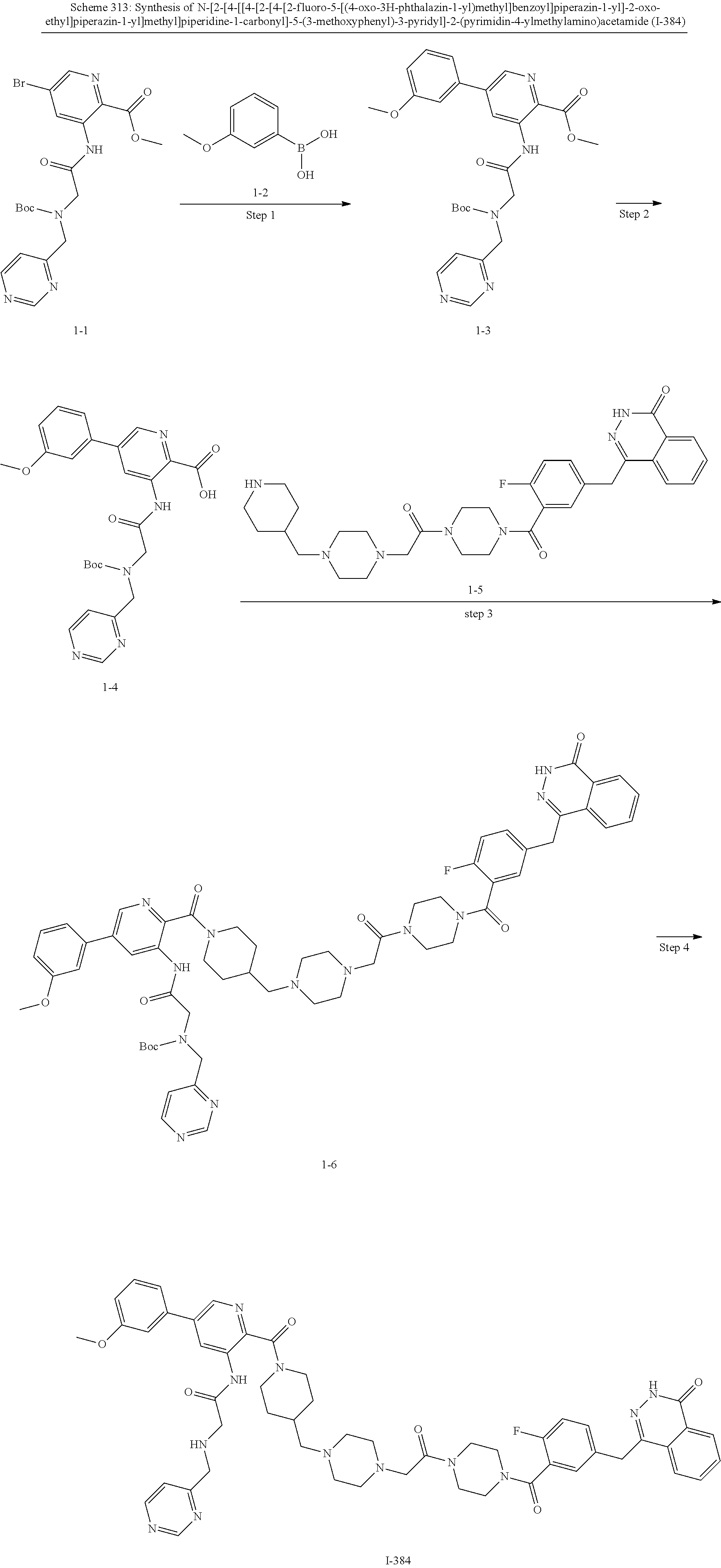

### Step 1: Synthesis of Intermediate 1-3

A mixture of Intermediate 1-2 (75.93 mg, 499.68 μmol, 1.2 eq), Intermediate 1-1 (200 mg, 416.40 μmol, 1 eq) and K₃PO₄ (265.16 mg, 1.25 mmol, 3 eq) in dioxane (2 mL) and H₂O (0.4 mL), then ditert-butyl(cyclopentyl)phosphane; dichloropalladium;iron (27.14 mg, 41.64 μmol, 0.1 eq) were added and the reaction mixture was purged with N₂ 3 times, and then the mixture was stirred at 80° C. for 1 h under N₂ atmosphere. The reaction mixture was washed with water (2 mL) and extracted with DCM (2 mL*3), the combined organic phase was dried by Na₂SO₄ and concentrated under reduced pressure to give a residue. The residue was purified by reversed-phase HPLC (0.1% of FA) and the eluent was lyophilized to give Intermediate 1-3 (80 mg, 157.31 μmol, 37.78% yield, 99.79% purity) as a white solid. LCMS (Method D): Retention time=0.427 min, (M+H)⁺=508.2.

### Step 2: Synthesis of Intermediate 1-4

A solution of Intermediate 1-3 (75 mg, 78.67 μmol, 1 eq, HCl salt) in H₂O (0.2 mL), THF (0.2 mL), and MeOH (0.2 mL) was added LiOH·H₂O (9.90 mg, 236.02 μmol, 3 eq). The reaction mixture was stirred and refluxed at 25° C. for 1 h. The mixture was concentrated under reduced pressure to give a residue. The crude product was used in the next step without further purification. Intermediate 1-4 (60 mg, crude) was obtained as a yellow solid. LCMS (Method D): Retention time=0.383 min, (M+H)⁺=494.2.

### Step 3: Synthesis of Intermediate 1-6

To a solution of Intermediate 1-5 (59.75 mg, 101.31 μmol, 1 eq) in DMF (0.5 mL) was added NMM (51.24 mg, 506.57 μmol, 55.69 μL, 5 eq) and EDCI (97.11 mg, 506.57 μmol, 5 eq), HOAt (27.58 mg, 202.63 μmol, 28.35 μL, 2 eq), Intermediate 1-4 (50 mg, 101.31 μmol, 1 eq). The mixture was stirred at 25° C. for 1 hr. The reaction mixture was washed with water (0.5 mL) and extracted with DCM (0.5 mL*3), the combined organic phase was dried by Na₂SO₄, and concentrated under reduced pressure to give a residue. The crude was purified by prep-HPLC (column: Phenomenex Luna C18 150*25 mm*10 um; mobile phase: [water(FA)-ACN]; gradient: 10%-40% B over 8 min) and the eluent was concentrated to remove ACN and lyophilized to give Intermediate 1-6 (30 mg, 28.16 μmol, 27.8% yield, 100% purity) as a white solid. LCMS (Method D): Retention time=0.307 min, (M+H)⁺=1065.2.

### Step 4: Synthesis of I-384

To a solution of Intermediate 1-6 (25 mg, 23.47 μmol, 1 eq) was added HCl/dioxane (2 M, 0.3 mL) and the mixture was stirred at 25° C. for 1 hr. The mixture was concentrated under reduced pressure to give a residue. The residue was purified by reversed-phase HPLC (0.1% HCl) and the eluent was lyophilized to give I-384 (10 mg, 9.71 μmol, 41.36% yield, 93.68% purity) as a yellow solid. LCMS (Method D): Retention time=0.300 min, (M+H)⁺=965.3. ¹H NMR (400 MHz, METHANOL-d4) δ=9.27 (s, 1H), 8.85 (d, J=5.2 Hz, 1H), 8.70 (s, 1H), 8.36 (d, J=5.2 Hz, 2H), 7.98-7.93 (m, 1H), 7.92-7.81 (m, 2H), 7.64 (d, J=5.2 Hz, 1H), 7.52-7.47 (m, 1H), 7.44 (t, J=8.0 Hz, 1H), 7.40-7.34 (m, 1H), 7.26 (d, J=8.0 Hz, 1H), 7.23 (s, 1H), 7.17 (t, J=8.8 Hz, 1H), 7.06-7.01 (m, 1H), 4.73-4.59 (m, 4H), 4.46 (d, J=14.4 Hz, 2H), 4.39 (s, 2H), 4.28 (s, 2H), 3.87 (s, 4H), 3.84 (s, 1H), 3.80 (s, 5H), 3.73 (d, J=1.6 Hz, 2H), 3.56 (s, 2H), 3.42 (s, 2H), 3.35 (s, 1H), 3.29-3.22 (m, 2H), 3.03-2.91 (m, 1H), 2.31 (s, 1H), 2.14-1.99 (m, 2H), 1.90 (d, J=12.0 Hz, 1H), 1.61 (d, J=8.8 Hz, 1H), 1.53-1.44 (m, 1H), 1.37-1.27 (m, 2H).

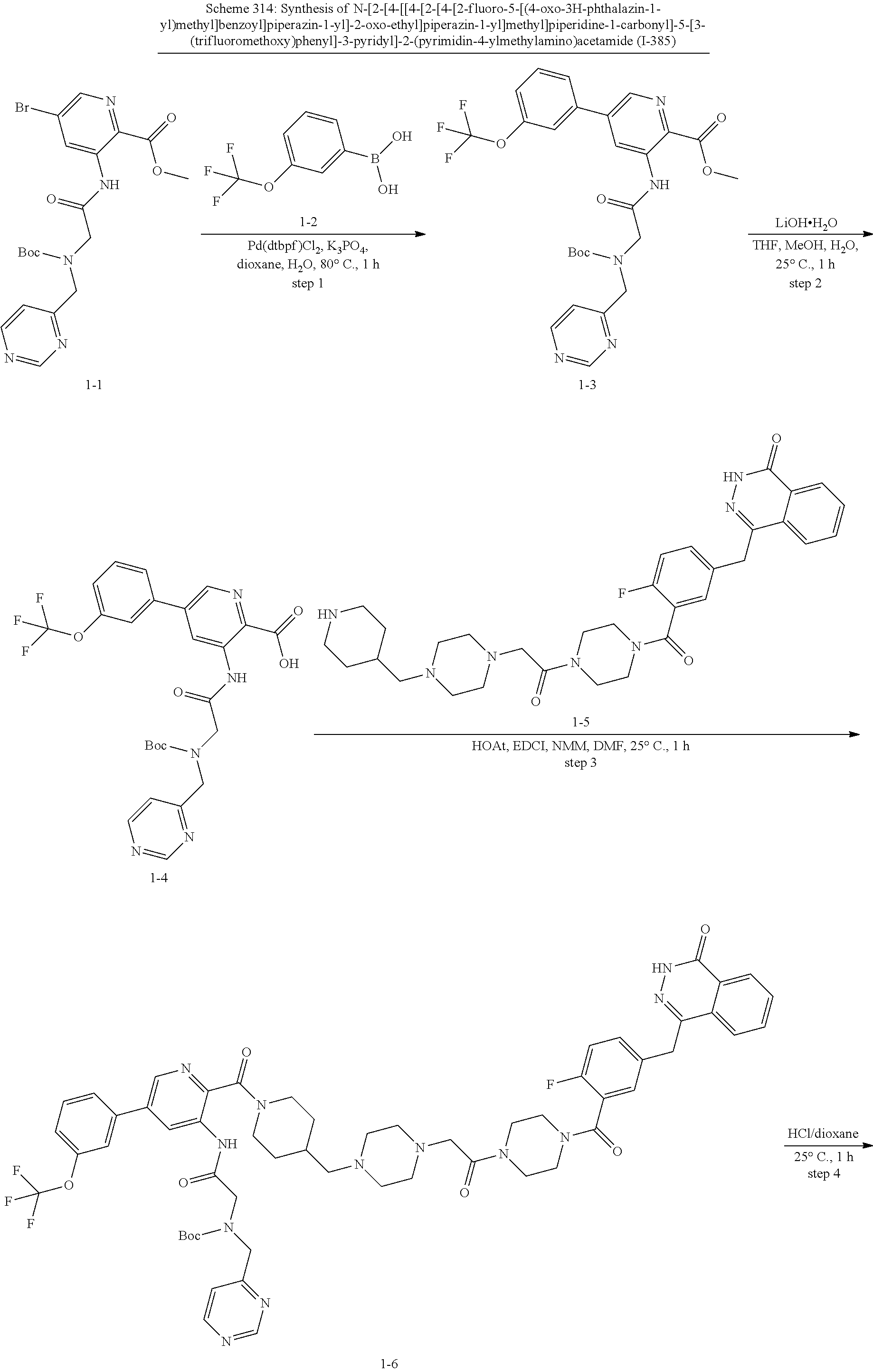

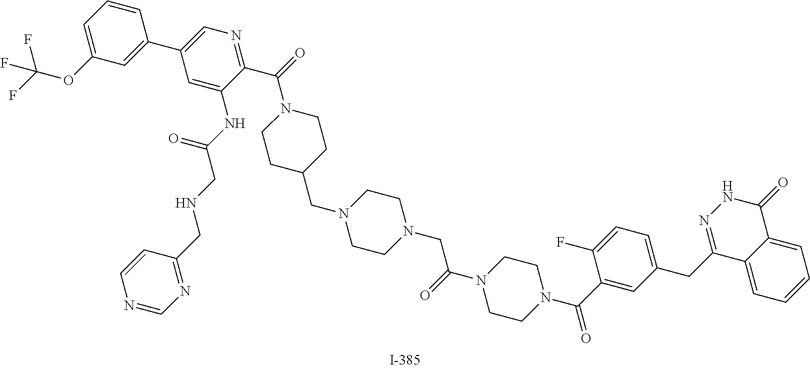 [SMILES: O=C(CNCc1ccncn1)Nc1cc(-c2cccc(OC(F)(F)F)c2)cnc1C(=O)N1CCC(CN2CCN(CC(=O)N3CCN(C(=O)c4cc(Cc5n[nH]c(=O)c6ccccc56)ccc4F)CC3)CC2)CC1]

### Step 1: Synthesis of Intermediate 1-3

A mixture of intermediate 1-1 (150 mg, 312.30 μmol, 1 eq), intermediate 1-2 (77.17 mg, 374.76 μmol, 1.2 eq), Pd(dtbpf)Cl₂ (20.35 mg, 31.23 μmol, 0.1 eq) and K₃PO₄ (198.87 mg, 936.89 μmol, 3 eq) in dioxane (1.5 mL) and H₂O (0.3 mL) was degassed and purged with N₂ 3 times, and then the mixture was stirred at 80° C. for 1 hr under N₂ atmosphere. The reaction mixture was poured into H₂O (2 mL) and extracted with DCM (5 mL*3). The combined organic layers were dried over Na₂SO₄, filtered and concentrated under reduced pressure to give the crude product. The residue was purified by flash silica gel chromatography (ISCO; 12 g SepaFlash Silica Flash Column, Eluent of 0˜100% Ethyl acetate/Petroleum ether gradient @40 mL/min) and concentrated under reduced pressure to give Intermediate 1-3 (160 mg, 284.95 μmol, 91.24% yield, 100% purity) as a brown oil. LCMS (Method D): Rt=0.538 min, (M+H)⁺=562.2.

### Step 2: Synthesis of Intermediate 1-4

To a mixture of intermediate 1-3 (110 mg, 195.90 μmol, 1 eq) in THF (0.4 mL), MeOH (0.4 mL) and H₂O (0.4 mL) was added LiOH H₂O (41.10 mg, 979.50 μmol, 5 eq). The mixture was stirred at 25° C. for 1 hr. The reaction mixture was adjusted to pH=7 with aq·HCl (1 M) and extracted with EA (2 mL*3). The combined organic layers were dried over Na₂SO₄, filtered and concentrated under reduced pressure to give the crude product. The crude product was used in the next step without further purification. Intermediate 1-4 (150 mg, crude) was obtained as a brown oil. LCMS (Method D): Rt=0.484 min, (M+H)⁺=548.2.

### Step 3: Synthesis of Intermediate 1-6

To a mixture of intermediate 1-4 (50 mg, 91.33 μmol, 1 eq) and intermediate 1-5 (62.90 mg, 100.46 μmol, 1.1 eq, HCl salt) in DMF (0.5 mL) was added EDCI (52.52 mg, 273.98 μmol, 3 eq), HOAT (12.43 mg, 91.33 μmol, 12.78 μL, 1 eq) and NMM (46.19 mg, 456.64 μmol, 50.20 μL, 5 eq). The mixture was stirred at 25° C. for 1 h. The reaction mixture was concentrated under reduced pressure to give a residue. The residue was purified by prep-HPLC (column: Welch Ultimate C18 150*25 mm*5 um; mobile phase: [water(FA)-ACN]; gradient: 17%-47% B over 10 min), and the eluent was lyophilized to give Intermediate 1-6 (20 mg, 17.69 μmol, 19.37% yield, 99% purity) as a white solid. LCMS (Method D): Rt=0.439 min, (M+H)⁺=1119.5.

### Step 4: Synthesis of I-385

A mixture of intermediate 1-6 (20 mg, 17.87 μmol, 1 eq) in added HCl/dioxane (2 M, 0.2 mL, 22.38 eq) was stirred at 25° C. for 1 hr. The reaction mixture was concentrated under reduced pressure to give I-385 (19.12 mg, 17.67 μmol, 98.85% yield, 97.52% purity, HCl salt) as a yellow solid. LCMS (Method D): Rt=0.371 min, (M+H)⁺=1019.4. ¹H NMR (400 MHz, METHANOL-d4) δ=9.31 (s, 1H), 8.89 (d, J=5.2 Hz, 1H), 8.76 (d, J=0.8 Hz, 1H), 8.47 (s, 1H), 8.37 (d, J=7.6 Hz, 1H), 7.99-7.93 (m, 1H), 7.92-7.81 (m, 2H), 7.75 (d, J=8.0 Hz, 1H), 7.70-7.63 (m, 3H), 7.54-7.47 (m, 1H), 7.45-7.35 (m, 2H), 7.17 (t, J=8.8 Hz, 1H), 4.70-4.64 (m, 3H), 4.58 (d, J=17.2 Hz, 2H), 4.39 (s, 2H), 4.29 (s, 2H), 3.92 (s, 5H), 3.88-3.77 (m, 5H), 3.73 (d, J=4.0 Hz, 1H), 3.66 (s, 1H), 3.56 (d, J=4.8 Hz, 2H), 3.41 (s, 2H), 3.35 (d, J=6.4 Hz, 3H), 3.25 (s, 1H), 3.01 (t, J=12.4 Hz, 1H), 2.39-2.28 (m, 1H), 2.10 (d, J=12.8 Hz, 1H), 1.93 (d, J=12.8 Hz, 1H), 1.68-1.58 (m, 1H), 1.57-1.46 (m, 1H). ¹⁹F NMR (376 MHz, METHAN OL-d4) δ=−59.388, −120.747.

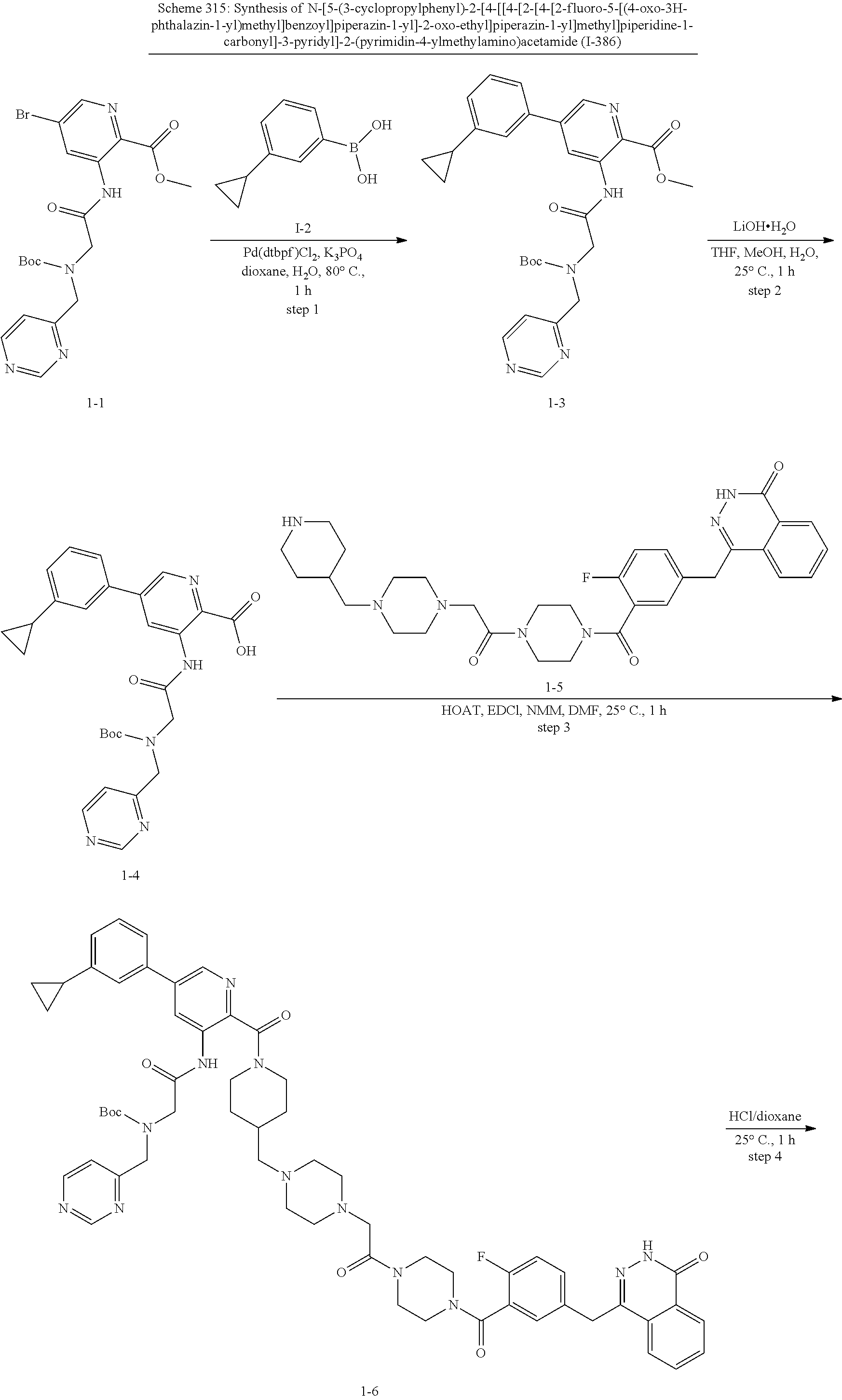

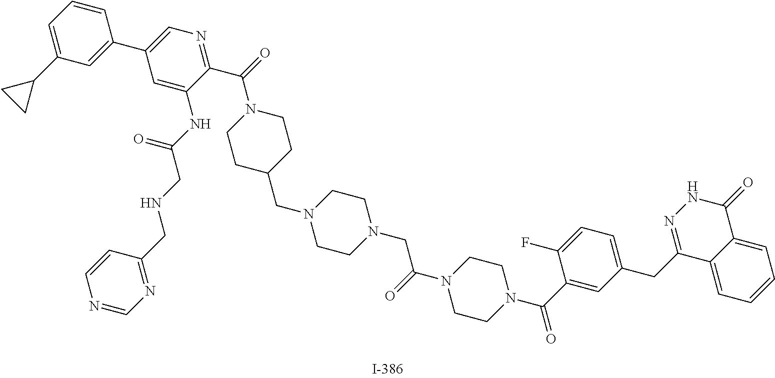 [SMILES: O=C(CNCc1ccncn1)Nc1cc(-c2cccc(C3CC3)c2)cnc1C(=O)N1CCC(CN2CCN(CC(=O)N3CCN(C(=O)c4cc(Cc5n[nH]c(=O)c6ccccc56)ccc4F)CC3)CC2)CC1]

### Step 1: Synthesis of Intermediate 1-3

A mixture of intermediate 1-1 (150 mg, 312.30 μmol, 1 eq), intermediate 1-2 (60.71 mg, 374.76 μmol, 1.2 eq), Pd(dtbpf)Cl₂ (20.35 mg, 31.23 μmol, 0.1 eq) and K₃PO₄ (198.87 mg, 936.89 μmol, 3 eq) in dioxane (1.5 mL) and H₂O (0.3 mL) was degassed and purged with N₂ 3 times, and then the mixture was stirred at 80° C. for 1 hr under N₂ atmosphere. The reaction was poured into H₂O (2 mL) and extracted with DCM (5 mL*3). The combined organic layers were dried over Na₂SO₄, filtered and concentrated under reduced pressure to give the crude product. The residue was purified by flash silica gel chromatography (ISCO; 12 g SepaFlash Silica Flash Column, Eluent of 0˜100% Ethyl acetate/Petroleum ether gradient @40 mL/min) and concentrated under reduced pressure to give a residue. Intermediate 1-3 (180 mg, crude) was obtained as a brown oil. LCMS (Method D): Rt=0.527 min, (M+H)=518.2.

### Step 2: Synthesis of Intermediate 1-4

To a mixture of intermediate 1-3 (130 mg, 251.17 μmol, 1 eq) in THF (0.2 mL), MeOH (0.2 mL) and H₂O (0.2 mL) was added LiOH H₂O (52.70 mg, 1.26 mmol, 5 eq). The mixture was stirred at 25° C. for 1 hr. The reaction mixture was adjusted to pH=7 with HCl (1 M) and extracted with EA (2 mL*3). The combined organic layers were dried over Na₂SO₄, filtered and concentrated under reduced pressure to give the crude product. The crude product was used in the next step without further purification. Intermediate 1-4 (135 mg, 249.33 μmol, 99.27% yield, 93% purity) was obtained as a brown oil. LCMS (Method D): Rt=0.467 min, (M+H)=504.7.

### Step 3: Synthesis of Intermediate 1-6

To a mixture of intermediate 1-4 (50 mg, 99.30 μmol, 1 eq) and intermediate 1-5 (68.39 mg, 109.22 μmol, 1.1 eq, HCl salt) in DMF (0.5 mL) was added EDCI (57.11 mg, 297.89 μmol, 3 eq), HOAT (13.52 mg, 99.30 μmol, 13.89 μL, 1 eq) and NMM (50.22 mg, 496.48 μmol, 54.58 μL, 5 eq). The mixture was stirred at 25° C. for 1 hr. The reaction mixture was concentrated under reduced pressure to give a residue. The residue was purified by prep-HPLC (column: Welch Ultimate C18 150*25 mm*5 um; mobile phase: [water(FA)-ACN]; gradient: 17%-47% B over 10 min) followed by lyophilization to give a residue. Intermediate 1-6 (30 mg, 27.34 μmol, 27.54% yield, 98% purity) was obtained as a white solid. LCMS (Method D): Rt=0.441 min, (1/2M+H)=538.5.

### Step 4: Synthesis of I-386

A mixture of intermediate 1-6 (30 mg, 27.90 μmol, 1 eq) in HCl/dioxane (2 M, 600.00 μL, 43.01 eq) was stirred at 25° C. for 1 h. The reaction mixture was concentrated under reduced pressure to give a residue. To the residue was added ACN and H₂O followed by lyophilization to give the product. I-386 (27.26 mg, 25.71 μmol, 92.14% yield, 95.397% purity, HCl salt) was obtained as a yellow solid. LCMS (Method D): Rt=0.362 min, (M+H)=975.5. ¹H NMR (400 MHz, METHANOL-d4) δ=9.35 (s, 1H), 8.92 (d, J=5.2 Hz, 1H), 8.82 (d, J=1.6 Hz, 1H), 8.62 (s, 1H), 8.37 (d, J=7.6 Hz, 1H), 7.99-7.93 (m, 1H), 7.92-7.87 (m, 1H), 7.86-7.81 (m, 1H), 7.75 (d, J=5.2 Hz, 1H), 7.53-7.48 (m, 2H), 7.47-7.42 (m, 2H), 7.41-7.36 (m, 1H), 7.22-7.14 (m, 2H), 4.71 (s, 2H), 4.69-4.61 (m, 2H), 4.57 (s, 1H), 4.39 (s, 2H), 4.35 (s, 2H), 3.94 (s, 5H), 3.86 (s, 2H), 3.79 (d, J=4.4 Hz, 2H), 3.74 (s, 2H), 3.66 (s, 1H), 3.56 (d, J=2.8 Hz, 2H), 3.42 (s, 2H), 3.37 (d, J=6.4 Hz, 4H), 3.06 (t, J=12.4 Hz, 1H), 2.40-2.30 (m, 1H), 2.10 (d, J=12.4 Hz, 1H), 2.06-1.99 (m, 1H), 1.95 (d, J=13.2 Hz, 1H), 1.65-1.49 (m, 2H), 1.07-1.01 (m, 2H), 0.80-0.76 (m, 2H). ¹⁹F NMR (376 MHz, METHANOL-d4) δ=−120.732.

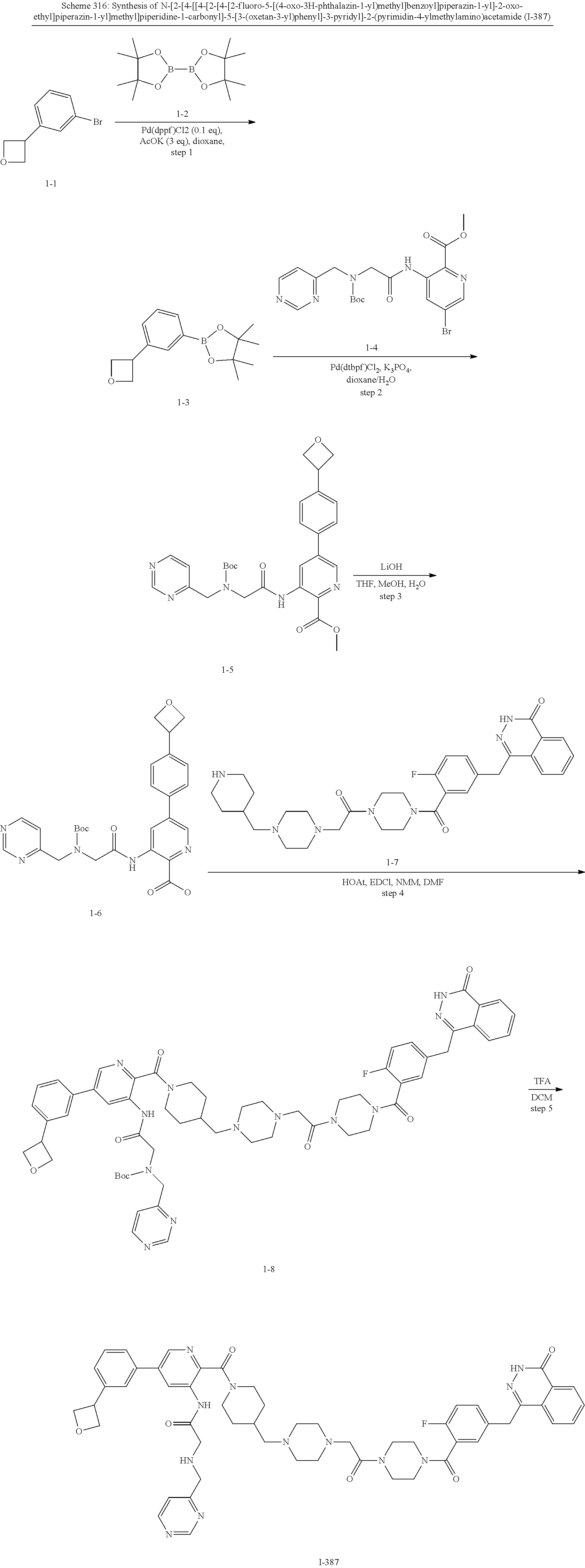

### Step 1: Synthesis of Intermediate 1-3

To a solution of intermediate 1-1 (500 mg, 2.35 mmol, 1 eq) and intermediate 1-2 (893.85 mg, 3.52 mmol, 1.5 eq) in dioxane (4 mL) was added AcOK (690.91 mg, 7.04 mmol, 3 eq) and Pd(dppf)Cl₂ (171.70 mg, 234.66 μmol, 0.1 eq). Then the mixture was stirred at 80° C. for 1 hour under N₂. To the reaction mixture was added H₂O (80 mL) and then it was extracted with EA (30 mL*2), the organic phase was dried by Na₂SO₄, filtered and concentrated. The residue was purified by flash silica gel chromatography (ISCO; 20 g SepaFlash Silica Flash Column, Eluent of 0˜30% Ethyl acetate/Petroleum ether gradient @80 mL/min) and the eluent was concentrated to give intermediate 1-3 (500 mg, 1.79 mmol, 76.17% yield, 93% purity) as a brown oil. LCMS (Method D): Retention time: 0.441 min, (M+H)=261.1. ¹H NMR (400 MHz, DMSO-d6) δ=7.68 (s, 1H), 7.59-7.54 (m, 2H), 7.43-7.37 (m, 1H), 4.97-4.94 (m, 2H), 4.61-4.58 (m, 2H), 4.30-4.23 (m, 1H), 1.31 (s, 12H).

### Step 2: Synthesis of Intermediate 1-5

To a solution of intermediate 1-3 (146.23 mg, 562.14 μmol, 1.5 eq) and intermediate 1-4 (180 mg, 374.76 μmol, 1 eq) in dioxane (2 mL) and H₂O (0.4 mL) was added ditert-butyl(cyclopentyl)phosphane;dichloropalladium;iron (24.42 mg, 37.48 μmol, 0.1 eq) and K₃PO₄ (238.64 mg, 1.12 mmol, 3 eq). The mixture was stirred at 80° C. for 1 hour under N₂. To the reaction mixture was added H₂O (80 mL) and then it was extracted with EA (30 mL*2), the organic phase was dried by Na₂SO₄, filtered and concentrated. The residue was purified by flash silica gel chromatography ((ISCO; 20 g SepaFlash Silica Flash Column, Eluent of 0˜10% Dichloromethane: Methanol @60 mL/min)). The eluent was concentrated under reduced pressure to give intermediate 1-5 (120 mg, 218.15 μmol, 58.21% yield, 97% purity) was obtained as a brown solid. LCMS (Method D): Retention time: 0.390 min, (M+H)=534.2. ¹H NMR (400 MHz, DMSO-d6) δ=11.15-10.85 (m, 1H), 9.11 (s, 1H), 8.93 (d, J=16.0 Hz, 1H), 8.77 (s, 2H), 7.77-7.49 (m, 5H), 5.01-4.97 (m, 2H), 4.75-4.61 (m, 4H), 4.37 (m, 1H), 4.28-4.17 (m, 2H), 3.90 (s, 3H), 1.37-1.26 (m, 9H).

### Step 3: Synthesis of Intermediate 1-6

To a solution of intermediate 1-5 (100 mg, 187.42 gmol, 1 eq) in THF (2 mL), H₂O (0.5 mL) and MeOH (1 mL) was added LiOH·H₂O (23.59 mg, 562.25 μmol, 3 eq). The mixture was stirred at 20° C. for 0.5 hr. The reaction mixture was concentrated under reduced pressure to remove THF and MeOH. The mixture was adjusted to pH=3-4 with 1M HCl and extracted with EA (10 mL*3). The combined organic layers were concentrated under reduced pressure to give a residue. Intermediate 1-6 (70 mg, 129.34 mol, 69.01% yield, 96% purity) was obtained as a brown solid. LCMS (Method D): Retention time: 0.371 min, (M+H)=520.1.

### Step 4: Synthesis of Intermediate 1-8

To a solution of intermediate 1-6 (45 mg, 86.61 μmol, 1 eq) and intermediate 1-7 (56.18 mg, 95.28 μmol, 1.1 eq) in DMF (1 mL) was added HOAt (11.79 mg, 86.61 μmol, 12.12 μL, 1 eq), EDCI (49.81 mg, 259.84 μmol, 3 eq) and NMM (43.80 mg, 433.07 μmol, 47.61 μL, 5 eq). Then the mixture was stirred at 40° C. for 1 hour. The reaction mixture was diluted with water (80 mL) and extracted with EA (30 mL*2). The combined organic layers were concentrated under reduced pressure to give a residue. The crude product was purified by reversed-phase HPLC (0.1% FA condition). The eluent was concentrated under reduced pressure to remove ACN and then lyophilized to give the product. Intermediate 1-8 (60 mg, 48.39 μmol, 55.86% yield, 88% purity) was obtained as a brown solid. LCMS (Method D): Retention time: 0.369 min, (M+H)=1091.7.

### Step 5: Synthesis of I-387

A solution of intermediate 1-8 (50 mg, 45.82 μmol, 1 eq) in DCM (1 mL) and TFA (0.2 mL) was stirred at 0° C. for 10 min. The reaction mixture was concentrated under reduced pressure to give a residue. The crude product was purified by reversed-phase column (0.1% NH₃·H₂O). The eluent was concentrated under reduced pressure to remove ACN and then lyophilized to give the product. I-387 (8.89 mg, 8.91 μmol, 19.45% yield, 99.347% purity) as a white solid. LCMS (Method D): Retention time: 0.303 min, (M+H)=991.5. ¹H NMR (400 MHz, METHANOL-d4) δ=9.10 (s, 1H), 8.83 (s, 1H), 8.79-8.74 (m, 1H), 8.65-8.62 (m, 1H), 8.39-8.34 (m, 1H), 7.98-7.92 (m, 1H), 7.90-7.86 (m, 1H), 7.85-7.81 (m, 1H), 7.80-7.76 (m, 1H), 7.73 (s, 1H), 7.65-7.60 (m, 1H), 7.57-7.53 (m, 2H), 7.51-7.47 (m, 1H), 7.40-7.35 (m, 1H), 7.20-7.13 (m, 1H), 5.17-5.12 (m, 2H), 4.83-4.80 (m, 4H), 4.62-4.57 (m, 2H), 4.42-4.37 (m, 3H), 4.01 (s, 2H), 3.81-3.71 (m, 4H), 3.68-3.64 (m, 1H), 3.58-3.54 (m, 1H), 3.53-3.49 (m, 3H), 3.26-3.20 (m, 2H), 3.19-3.09 (m, 2H), 2.89-2.82 (m, 1H), 2.55-2.44 (m, 6H), 2.26-2.19 (m, 2H), 1.91-1.84 (m, 2H), 1.78-1.72 (m, 1H), 1.35-1.29 (m, 1H), 1.18-1.11 (m, 1H). ¹⁹F NMR (400 MHz, METHANOL-d4) δ=−120.770.

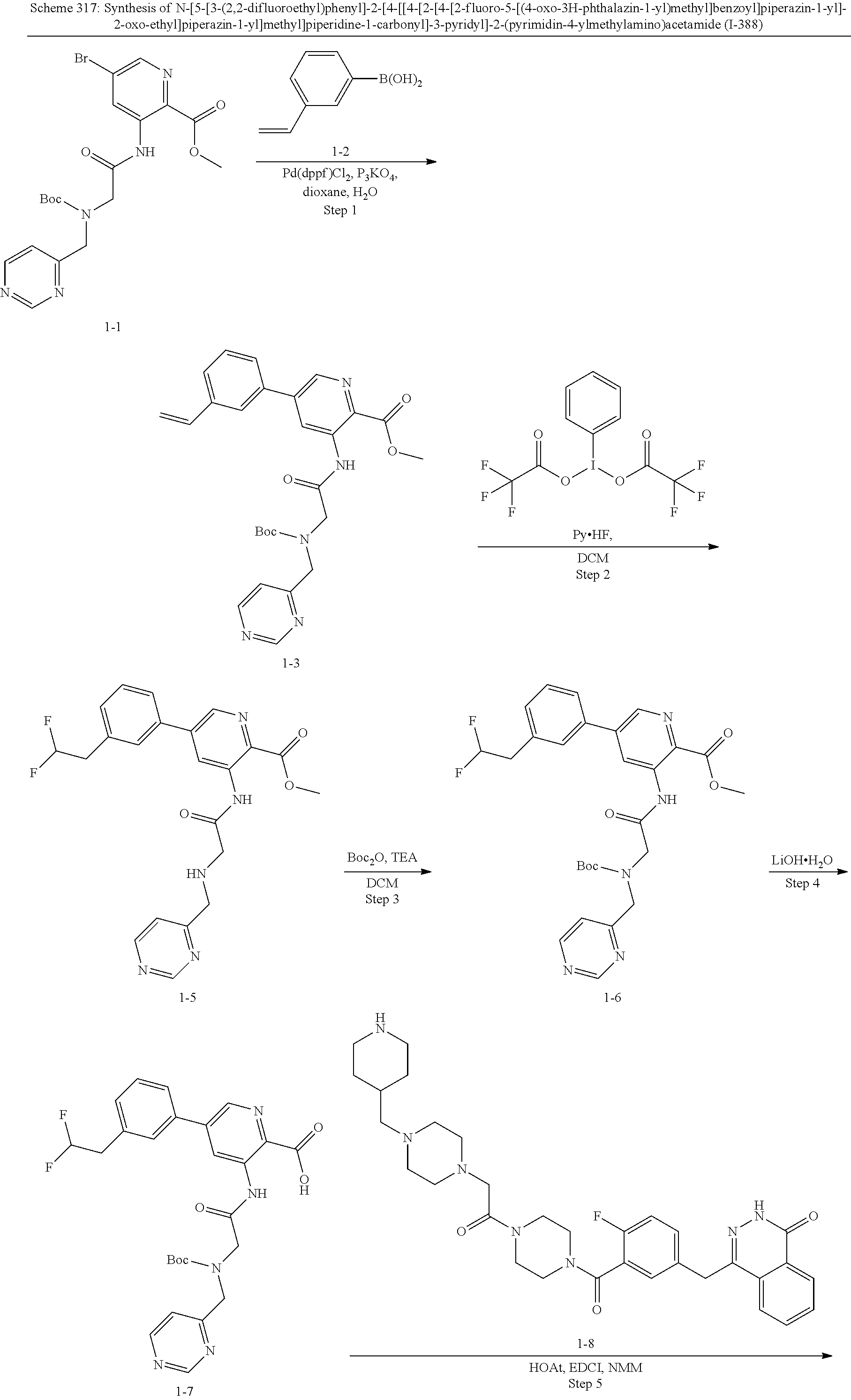

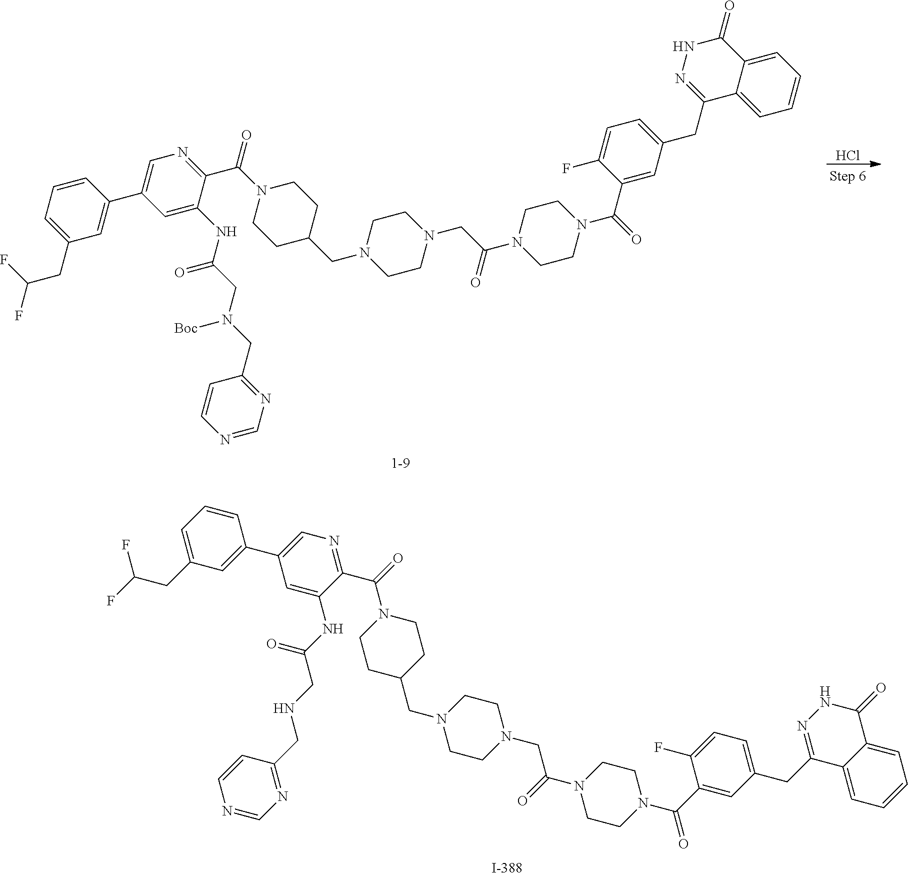 [SMILES: CC(C)(C)OC(=O)N(CC(=O)Nc1cc(-c2cccc(CC(F)F)c2)cnc1C(=O)N1CCC(CN2CCN(CC(=O)N3CCN(C(=O)c4cc(Cc5n[nH]c(=O)c6ccccc56)ccc4F)CC3)CC2)CC1)Cc1ccncn1 ; Cl ; O=C(CNCc1ccncn1)Nc1cc(-c2cccc(CC(F)F)c2)cnc1C(=O)N1CCC(CN2CCN(CC(=O)N3CCN(C(=O)c4cc(Cc5n[nH]c(=O)c6ccccc56)ccc4F)CC3)CC2)CC1]

### Step 1: Synthesis of Intermediate 1-3.

A mixture of Intermediate 1-1 (0.5 g, 1.04 mmol, 1 eq) and Intermediate 1-2 (231.05 mg, 1.56 mmol, 1.5 eq) in H₂O (1.2 mL) and dioxane (6 mL) was added K₃PO₄ (662.91 mg, 3.12 mmol, 3 eq) and Pd(dppf)Cl₂ (76.17 mg, 104.10 μmol, 0.1 eq), then the mixture was stirred at 80° C. for 1 hour under N₂ atmosphere. The reaction mixture was diluted with water (50 mL) and extracted with EA (10 mL*3). The combined organic layers were concentrated under reduced pressure to give a residue. The residue was purified by flash silica gel chromatography (ISCO; 40 g SepaFlash Silica Flash Column, Eluent of 0˜30% Ethyl acetate/Petroleum ether gradient @80 mL/min). The eluent was concentrated under reduced pressure to give Intermediate 1-3 (300 mg, 595.77 μmol, 57.23% yield) as a white solid. LCMS (Method D): Retention time: 0.459 min, (M+H)=504.3.

### Step 2: Synthesis of Intermediate 1-5.

To a mixture of Intermediate 1-3 (220 mg, 436.90 μmol, 1 eq) in DCM (10 mL) was added Intermediate 1-4 (206.67 mg, 480.59 μmol, 1.1 eq), pyridine;hydrofluoride (1.24 g, 8.74 mmol, 1.12 mL, 70% purity, 20 eq), then the mixture was stirred at 20° C. for 1 hour. To the reaction mixture was added a NaHCO₃ solution (about 3 mL) to adjust the pH to 7-8. The mixture was extracted with EA (10 mL*3). The combined organic layers were concentrated under reduced pressure to give a residue. The crude product was purified by reversed-phase HPLC (0.1% FA condition). The eluent was concentrated under reduced pressure to remove MeCN and then lyophilized to give Intermediate 1-5 (80 mg, 164.37 μmol, 37.62% yield, 90.7% purity) as a white solid. LCMS (Method D): Retention time: 0.305 min, (M+H)=442.1. ¹H NMR (400 MHz, CHLOROFORM-d) δ=12.06 (s, 1H), 9.47-9.38 (m, 1H), 9.22 (d, J=1.2 Hz, 1H), 8.74 (d, J=5.2 Hz, 1H), 8.70-8.65 (m, 1H), 7.61 (d, J=8.0 Hz, 1H), 7.56-7.52 (m, 2H), 7.51-7.47 (m, 1H), 7.37 (d, J=7.6 Hz, 1H), 6.16-5.83 (m, 1H), 4.15 (s, 2H), 4.04 (s, 3H), 3.71 (s, 2H), 3.27-3.31 (m 2H).

### Step 3: Synthesis of Intermediate 1-6.

To a mixture of Intermediate 1-5 (70 mg, 158.58 μmol, 1 eq) in DCM (2 mL) was added TEA (48.14 mg, 475.73 μmol, 66.22 μL, 3 q) and Boc₂O (51.91 mg, 237.86 μmol, 54.65 μL, 1.5 eq) at 0° C. Then the mixture was stirred at 20° C. for 1 hour. The reaction mixture was diluted with water (30 mL) and extracted with EA (5 mL*3). The combined organic layers were concentrated under reduced pressure to give a residue. The residue was purified by flash silica gel chromatography (ISCO; 12 g SepaFlash Silica Flash Column, Eluent of 0˜30% ethyl acetate/Petroleum ether gradient @30 mL/min). The eluent was on concentrated under reduced pressure to give Intermediate 1-6 (60 mg, 110.79 μmol, 69.87% yield) as a white solid. LCMS (Method D): Retention time: 0.456 min, (M+H)=542.4.

### Step 4: Synthesis of Intermediate 1-7.

To a mixture of Intermediate 1-6 (60 mg, 110.79 μmol, 1 eq) in THF (2 mL), MeOH (1 mL), and H₂O (0.5 mL) was added LiOH·H₂O (9.30 mg, 221.59 μmol, 2 eq), then the mixture was stirred at 20° C. for 0.5 hour. The reaction mixture was concentrated under reduced pressure to remove THF, and then 1 N HCl was added (about 3 mL) to adjust the pH to 3-4. The mixture was then extracted with EA (10 mL*3). The combined organic layers were concentrated under reduced pressure to give Intermediate 1-7 (60 mg, crude) as a white solid. LCMS (Method D): Retention time: 0.408 min, (M+H)=528.2.

### Step 5: Synthesis of Intermediate 1-9.

To a mixture of Intermediate 1-7 (50 mg, 94.78 μmol, 1 eq) and Intermediate 1-8 (67.07 mg, 113.74 μmol, 1.2 eq) in DMF (1 mL) was added HOAt (12.90 mg, 94.78 μmol, 13.26 μL, 1 eq), EDCI (54.51 mg, 284.35 μmol, 3 eq), NMM (47.94 mg, 473.92 μmol, 52.10 μL, 5 eq) then the mixture was stirred at 20° C. for 1 hour. The reaction mixture was diluted with water (50 mL) and extracted with EA (50 mL*2). The combined organic layers were concentrated under reduced pressure to give Intermediate 1-9 (60 mg, crude) as a white solid. LCMS (Method D): Retention time: 0.357 min, (M+H)=1099.2.

### Step 6: Synthesis of I-388

A mixture of Intermediate 1-9 (40 mg, 36.39 μmol, 1 eq) in HCl/dioxane (1 mL) was stirred at 20° C. for 5 min. The reaction mixture was concentrated under reduced pressure to give a residue. The crude product was purified by reversed-phase HPLC (0.1% FA condition). The eluent was concentrated under reduced pressure to remove MeCN and then lyophilized to give I-388 (34.19 mg, 32.06 μmol, 88.10% yield, 98% purity, FA salt) as a pink solid. LCMS (Method D): Retention time: 0.310 min, (M+H)=999.5. ¹H NMR (400 MHz, METHANOL-d4) δ=9.11 (s, 1H), 8.82-8.72 (m, 2H), 8.64-8.60 (m, 1H), 8.37 (d, J=7.6 Hz, 1H), 8.28 (s, 1H), 7.97-7.92 (m, 1H), 7.89-7.81 (m, 2H), 7.77 (d, J=5.4 Hz, 1H), 7.64 (s, 2H), 7.53-7.48 (m, 2H), 7.42-7.33 (m, 2H), 7.20-7.14 (m, 1H), 6.26-5.93 (m, 1H), 4.66-4.59 (m, 1H), 4.39 (s, 2H), 4.03 (s, 2H), 3.88-3.74 (m, 3H), 3.70-3.63 (m, 2H), 3.61-3.44 (m, 7H), 3.38-3.34 (m, 1H), 3.27-3.13 (m, 3H), 3.03-2.82 (m, 9H), 2.68 (s, 2H), 2.08-1.98 (m, 1H), 1.93-1.85 (m, 1H), 1.80-1.73 (m, 1H), 1.43-1.32 (m, 1H), 1.30-1.22 (m, 1H). ¹⁹F NMR (376 MHz, METHANOL-d4) δ=−116.99, δ=−120.78.

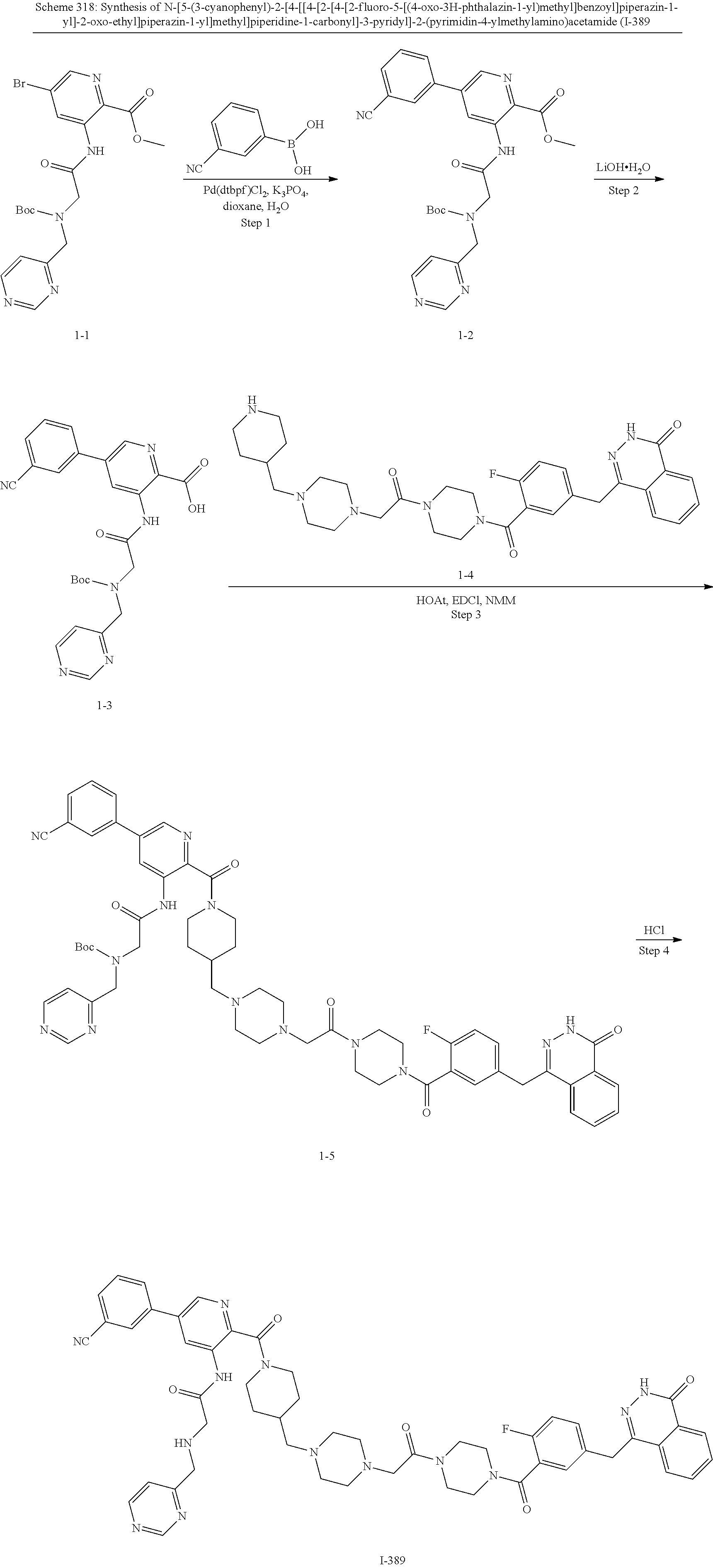

### Step 1: Synthesis of Intermediate 1-2.

To a mixture of Intermediate 1-1 (180 mg, 374.76 μmol, 1 eq) and (3-cyanophenyl)boronic acid (82.60 mg, 562.14 μmol, 1.5 eq) in dioxane (2 mL) and H₂O (0.4 mL) was added K₃PO₄ (238.64 mg, 1.12 mmol, 3 eq) and ditert-butyl(cyclopentyl) phosphane; dichloropalladium; iron (24.42 mg, 37.48 μmol, 0.1 eq), then the mixture was stirred at 80° C. for 1 hour under N₂. The reaction mixture was diluted with water (50 mL) and extracted with EA (10 mL*3). The combined organic layers were concentrated under reduced pressure to give a residue. The residue was purified by flash silica gel chromatography (ISCO; 20 g SepaFlash Silica Flash Column, Eluent of 0˜100% Ethyl acetate/Petroleum ether gradient @60 mL/min). The eluent was concentrated under reduced pressure to give Intermediate 1-2 (180 mg, 358.19 μmol, 95.58% yield) as a white solid. LCMS (Method D): Retention time: 0.418 min, (M+H)=503.0.

### Step 2: Synthesis of Intermediate 1-3.

To a mixture of Intermediate 1-2 (180 mg, 358.19 μmol, 1 eq) in THF (2 mL), MeOH (1 mL), and H₂O (0.5 mL) was added LiOH·H₂O (45.09 mg, 1.07 mmol, 3 eq), then the mixture was stirred at 20° C. for 1 hour. The reaction mixture was concentrated under reduced pressure to remove THF, and then 1 N HCl was added (about 3 mL) to adjust pH to 3-4. The mixture was extracted with EA (10 mL*3). The combined organic layers were concentrated under reduced pressure to give Intermediate 1-3 (100 mg, 204.71 μmol, 57.15% yield) as a white solid. LCMS (Method D): Retention time: 0.365 min, (M+H)=489.2.

### Step 3: Synthesis of Intermediate 1-5.

To a mixture of Intermediate 1-3 (50 mg, 102.36 μmol, 1 eq) and Intermediate 1-4 (60.36 mg, 102.36 μmol, 1 eq) in DMF (1 mL) was added HOAt (13.93 mg, 102.36 μmol, 14.32 μL, 1 eq), EDCI (58.86 mg, 307.07 μmol, 3 eq), and NMM (51.76 mg, 511.78 μmol, 56.27 μL, 5 eq) and it was stirred at 20° C. for 1 hour. The reaction mixture was diluted with water (50 mL) and extracted with EA (15 mL*2). The combined organic layers were concentrated under reduced pressure to give Intermediate 1-5 (80 mg, 75.46 μmol, 73.72% yield) as a white solid. LCMS (Method D): Retention time: 0.364 min, (M+H)=1060.4.

### Step 4: Synthesis of I-389

A mixture of Intermediate 1-5 (50 mg, 47.16 μmol, 1 eq) in DCM (1 mL) was added HCl/dioxane (2 M, 117.90 μL, 5 eq) then the mixture was stirred at 20° C. for 10 min. The reaction mixture was concentrated under reduced pressure to give a residue. The crude product was purified by reversed-phase HPLC (0.1% FA condition). The eluent was concentrated under reduced pressure to remove MeCN and then lyophilized to give I-389 (20.16 mg, 20.84 μmol, 44.19% yield, 99.24% purity) as an off-white solid. LCMS (Method D): Retention time: 0.298 min, (M+H)=960.3. ¹H NMR (400 MHz, METHANOL-d4) 6=9.10 (s, 1H), 8.82 (d, J=1.6 Hz, 1H), 8.77 (d, J=5.6 Hz, 1H), 8.66 (d, J=2.0 Hz, 1H), 8.41 (s, 1H), 8.38-8.35 (m, 1H), 8.12 (s, 1H), 8.03 (d, J=8.0 Hz, 1H), 7.97-7.92 (m, 1H), 7.91-7.80 (m, 3H), 7.78 (d, J=5.2 Hz, 1H), 7.75-7.69 (m, 1H), 7.50 (d, J=2.8 Hz, 1H), 7.41-7.33 (m, 1H), 7.19-7.14 (m, 1H), 4.61 (d, J=13.2 Hz, 1H), 4.39 (s, 2H), 4.02 (s, 2H), 3.86-3.72 (m, 3H), 3.68 (d, J=5.4 Hz, 2H), 3.58-3.50 (m, 4H), 3.46 (s, 1H), 3.40-3.32 (m, 3H), 3.18-3.10 (m, 1H), 2.93-2.62 (m, 9H), 2.50-2.47 (m, 2H), 2.05-1.93 (m, 1H), 1.93-1.85 (m, 1H), 1.76 (d, J=12.0 Hz, 1H), 1.38-1.30 (m, 1H), 1.28-1.16 (m, 1H). ¹⁹F NMR (376 MHz, CHLOROFORM-d) δ=−120.78.

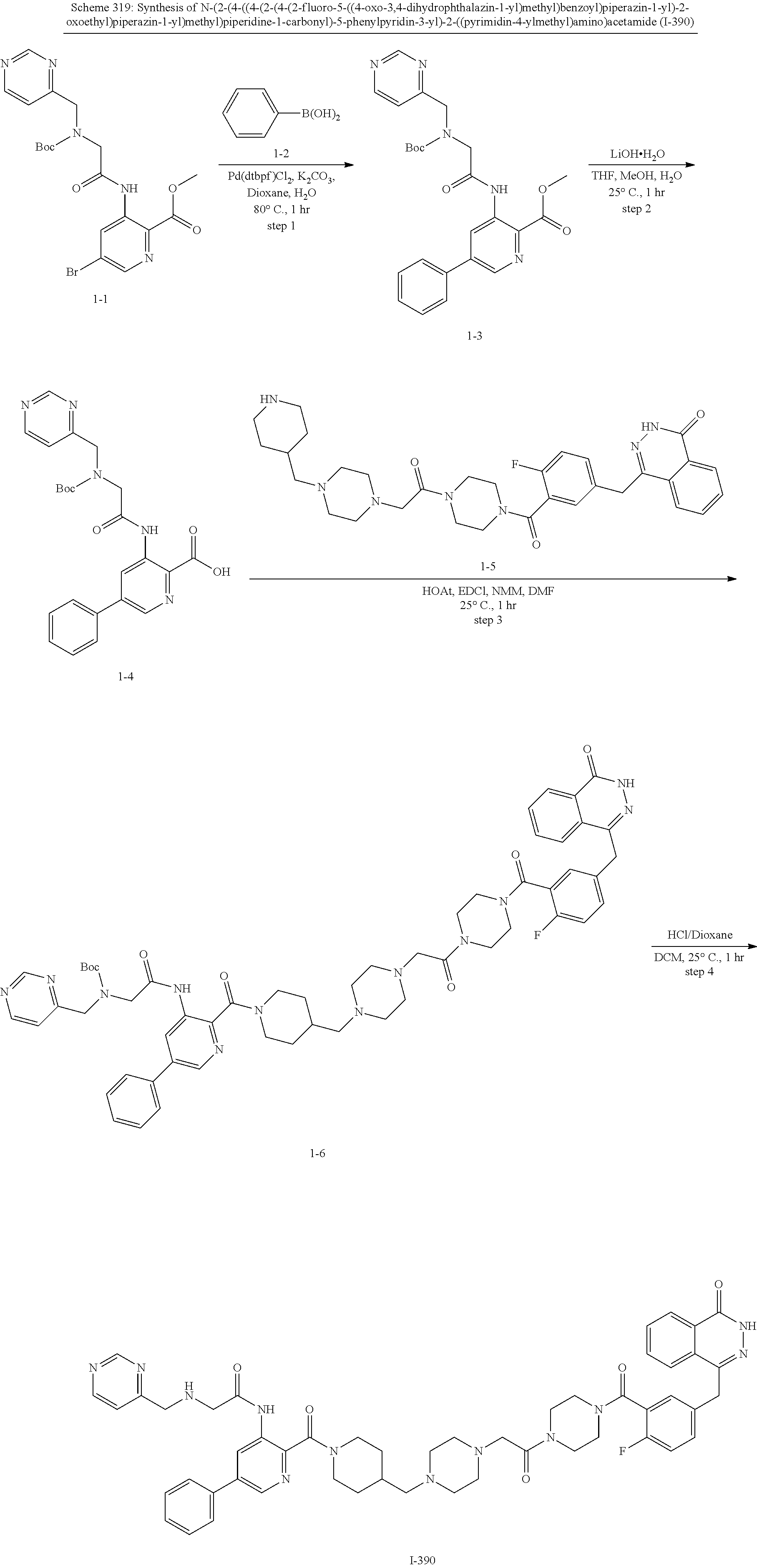

### Step 1: Synthesis of Intermediate 1-3

A mixture of Intermediate 1-1 (170 mg, 353.94 μmol, 1 eq), Intermediate 1-2 (51.79 mg, 424.72 μmol, 1.2 eq), Pd(dtbpf)Cl₂ (23.07 mg, 35.39 μmol, 0.1 eq) and K₃PO₄ (225.39 mg, 1.06 mmol, 3 eq) in dioxane (1.5 mL) and H₂O (0.3 mL) was degassed and purged with N₂ 3 times, and then the mixture was stirred at 80° C. for 1 hr under N₂ atmosphere. The mixture was diluted with water (1 mL) and extract with EA (2 mL*3). The combined organic layers were dried over sodium sulfate, filtered and concentrated under reduced pressure to give a residue. The residue was purified by flash silica gel chromatography (ISCO; 20 g SepaFlash Silica Flash Column, Eluent of 0˜100% PE/EA gradient @80 mL/min) and then the eluent was concentrated in vacuo to give Intermediate 1-3 (170 mg, 345.33 μmol, 97.57% yield, 97% purity) as yellow oil. LCMS (Method D): Rt: 0.463 min, (M+H)=478.3. ¹H NMR (400 MHz, METHANOL-d4) δ=9.30 (s, 1H), 9.08 (s, 1H), 8.80-8.60 (m, 2H), 7.72 (d, J=7.2 Hz, 2H), 7.57-7.45 (m, 4H), 4.76 (d, J=6.8 Hz, 2H), 4.34-4.25 (m, 2H), 4.01 (s, 3H), 1.41 (d, J=9.2 Hz, 9H).

### Step 2: Synthesis of Intermediate 1-4

To a solution of Intermediate 1-3 (140 mg, 293.19 μmol, 1 eq) in THF (0.5 mL), H₂O (0.5 mL) and MeOH (0.5 mL) was added LiOH·H₂O (36.91 mg, 879.56 μmol, 3 eq). The mixture was stirred at 25° C. for 1 hr. The pH was acidified to 3-4 with 1N HCl, and the mixture was diluted with water (5 mL) and extracted with EA (3 mL*3). The combined organic layers were dried over sodium sulfate, filtered and concentrated under reduced pressure to give a residue. The residue was purified by flash silica gel chromatography (ISCO; 20 g SepaFlash Silica Flash Column, Eluent of 0˜10% EA/MeOH gradient @50 mL/min) and then the eluent was concentrated in vacuo to give Intermediate 1-4 (130 mg, 272.07 μmol, 92.80% yield, 97% purity) as yellow oil. LCMS (Method D): Rt: 0.374 min, (M+H)=464.2.

### Step 3: Synthesis of Intermediate 1-6

To a solution of Intermediate 1-4 (110 mg, 237.33 μmol, 1 eq) in DMF (1 mL) was added HOAt (32.30 mg, 237.33 μmol, 33.20 μL, 1 eq), EDCI (136.49 mg, 712.00 μmol, 3 eq), NMM (120.03 mg, 1.19 mmol, 130.46 μL, 5 eq) and Intermediate 1-5 (148.61 mg, 237.33 μmol, 1 eq, HCl salt). The mixture was stirred at 25° C. for 1 hr. The mixture was diluted with water (3 mL) and extracted with EA (2 mL*3). The combined organic layers were dried over sodium sulfate, filtered and concentrated under reduced pressure to give a residue. The residue was purified by reversed phase HPLC (FA condition). The eluent was concentrated under reduced pressure to remove ACN and lyophilized to give Intermediate 1-6 (75 mg, 65.93 μmol, 27.78% yield, 91% purity) as a white solid. LCMS (Method D): Rt: 0.381 min, (M+H)=1035.6.

### Step 4: Synthesis of I-390

To a solution of Intermediate 1-6 (65 mg, 62.79 μmol, 1 eq) in DCM (0.7 mL) was added HCl/dioxane (2 M, 0.7 mL). The mixture was stirred at 25° C. for 1 hr. The pH was basified to 7-8 with NH₃·H₂O, and the mixture was washed with water (3 mL) and extracted with EA (2 mL*3). The combined organic layers were dried over sodium sulfate, filtered and concentrated under reduced pressure to give a residue. The residue was purified by reversed phase HPLC (FA condition), and the eluent was concentrated under reduced pressure to remove ACN and lyophilized to give I-390 (8.78 mg, 8.76 μmol, 13.95% yield, 97.898% purity, FA salt) as yellow solid. LCMS (Method D): Rt: 0.285 min, (M+H)=935.4. ¹H NMR (400 MHz, METHANOL-d4) δ=9.10 (s, 1H), 8.80-8.75 (m, 2H), 8.62 (d, J=2.0 Hz, 1H), 8.38 (s, 1H), 8.36 (d, J=1.2 Hz, 1H), 7.94 (d, J=7.2 Hz, 1H), 7.90-7.82 (m, 2H), 7.78 (d, J=5.2 Hz, 1H), 7.71 (d, J=7.6 Hz, 2H), 7.54-7.46 (m, 4H), 7.39-7.34 (m, 1H), 7.19-7.13 (m, 1H), 4.61 (d, J=12.4 Hz, 1H), 4.39 (s, 2H), 4.02 (s, 2H), 3.88-3.71 (m, 4H), 3.67 (s, 2H), 3.56-3.47 (m, 5H), 3.45 (s, 1H), 3.37 (s, 1H), 3.19-3.13 (m, 1H), 2.90-2.85 (m, 1H), 2.79-2.64 (m, 8H), 2.47 (m, 2H), 1.98-1.86 (m, 2H), 1.76 (d, J=14.4 Hz, 1H), 1.37-1.30 (m, 1H), 1.24-1.14 (m, 1H). ¹⁹F NMR (376 MHz, METHANOL-d4) δ=−1200.76.

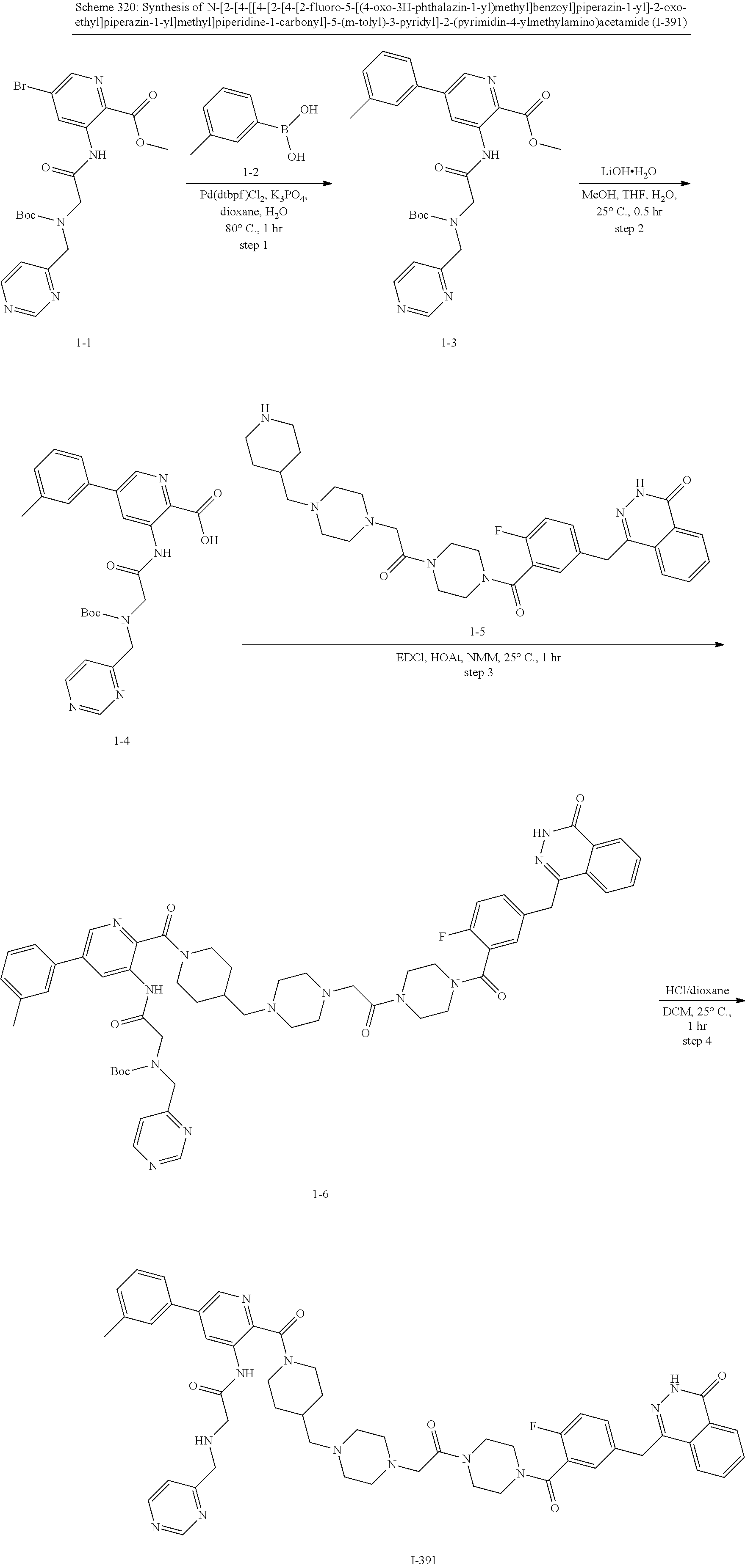

### Step 1: Synthesis of Intermediate 1-3.

To a solution of Intermediate 1-1 (170 mg, 353.94 μmol, 1 eq) in H₂O (0.4 mL) and 1,4-dioxane (2 mL) was added Intermediate 1-2 (57.74 mg, 424.72 μmol, 1.2 eq), K₃PO₄ (225.39 mg, 1.06 mmol, 3 eq), and Pd(dtbpf)Cl₂ (23.07 mg, 35.39 μmol, 0.1 eq). The mixture was stirred at 80° C. for 1 hr under N₂. The reaction mixture diluted with H₂O (2 mL) and extracted with EA (2 mL*2). The combined organic layers were dried over Na₂SO₄, filtered and concentrated under reduced pressure to give a residue. The residue was purified by flash silica gel chromatography (ISCO; 12 g SepaFlash Silica Flash Column, Eluent of 0-100% Ethylacetate/Petroleum ether gradient @60 mL/min). The eluent was concentrated under reduced pressure to give Intermediate 1-3 (150 mg, 294.18 μmol, 83.12% yield, 96.4% purity) was obtained as yellow oil. LCMS (Method D): Retention time: 0.459 min, (M+H)=492.2. ¹HNMR (400 MHz, METHANOL-d4) δ=9.30 (d, J=7.6 Hz, 1H), 9.09 (s, 1H), 8.75 (d, J=4.8 Hz, 1H), 8.64 (s, 1H), 7.60-7.49 (m, 3H), 7.47-7.39 (m, 1H), 7.32 (d, J=7.6 Hz, 1H), 4.77 (d, J=6.0 Hz, 2H), 4.36-4.24 (m, 2H), 4.02 (d, J=4.4 Hz, 3H), 2.45 (s, 3H), 1.41 (d, J=9.6 Hz, 9H).

### Step 2: Synthesis of Intermediate 1-6.

To a solution of Intermediate 1-3 (120 mg, 244.13 μmol, 1 eq) in MeOH (0.5 mL), THF (0.5 mL), and H₂O (0.5 mL) was added LiOHH₂O (30.73 mg, 732.40 μmol, 3 eq). The mixture was stirred at 25° C. for 0.5 hr. The mixture was diluted with H₂O (2 mL) and HCl (1M) was added to adjust pH to 4. The mixture was extracted with EA (2 mL*2). The combined organic layers were dried over Na₂SO₄, filtered and concentrated under reduced pressure to give a residue. The crude product Intermediate 1-4 (140 mg, crude) was obtained as a yellow gum and was used in the next step without further purification. LCMS (Method D): Retention time: 0.402 min, (M+H)=478.2.

### Step 3: Synthesis of Intermediate 1-6.

To a solution of Intermediate 1-4 (100 mg, 209.42 μmol, 1 eq) in DMF (1 mL) was added Intermediate 1-5 (157.36 mg, 251.30 μmol, 1.2 eq, HCl salt), EDCI (120.44 mg, 628.26 μmol, 3 eq), HOAt (28.50 mg, 209.42 μmol, 29.30 μL, 1 eq) and NMM (105.91 mg, 1.05 mmol, 115.12 μL, 5 eq). The mixture was stirred at 25° C. for 1 hr. The mixture was filtered. The filtrate was purified by reversed-phase HPLC (0.1% FA condition). The eluent was concentrated under reduced pressure to remove ACN and then lyophilized to give Intermediate 1-6 (50 mg, 47.66 μmol, 22.76% yield) as a white solid. LCMS (Method D): Retention time: 0.386 min, (M+H)=1049.5.

### Step 4: Synthesis of I-391

A solution of Intermediate 1-6 (40 mg, 38.12 μmol, 1 eq) in DCM (0.2 mL) and HCl/dioxane (2 M, 0.4 mL) was stirred at 25° C. for 1 hr. The mixture was concentrated in vacuum. The crude product was purified by reversed-phase HPLC (0.1% FA condition). The eluent was concentrated under reduced pressure to remove ACN and then lyophilized to give I-391 (11.05 mg, 11.50 μmol, 30.17% yield, 98.8% purity) as a pink solid. LCMS (Method D): Retention time: 0.313 min, (M+H)=949.4. ¹H NMR (400 MHz, METHANOL-d₄) δ=9.10 (s, 1H), 8.77 (d, J=5.2 Hz, 1H), 8.74 (d, J=1.6 Hz, 1H), 8.60 (d, J=2.0 Hz, 1H), 8.41 (s, 1H), 8.38-8.32 (m, 1H), 7.98-7.91 (m, 1H), 7.90-7.80 (m, 2H), 7.80-7.74 (m, 1H), 7.54-7.45 (m, 3H), 7.43-7.33 (m, 2H), 7.28 (d, 1H), 7.20-7.12 (m, 1H), 4.62-4.38 (m, 1H), 4.38 (s, 2H), 4.02 (s, 2H), 3.88-3.72 (m, 3H), 3.67 (s, 2H), 3.56-3.47 (m, 5H), 3.43 (s, 1H), 3.38-3.31 (m, 2H), 3.21-3.11 (m, 1H), 2.94-2.71 (m, 9H), 2.62-2.53 (m, 2H), 2.43 (s, 3H), 2.06-1.94 (m, 1H), 1.93-1.83 (m, 1H), 1.82-1.69 (m, 1H), 1.42-1.15 (m, 2H). ¹⁹FNMR (376 MHz, MeOD-d₆) δ=−120.697.

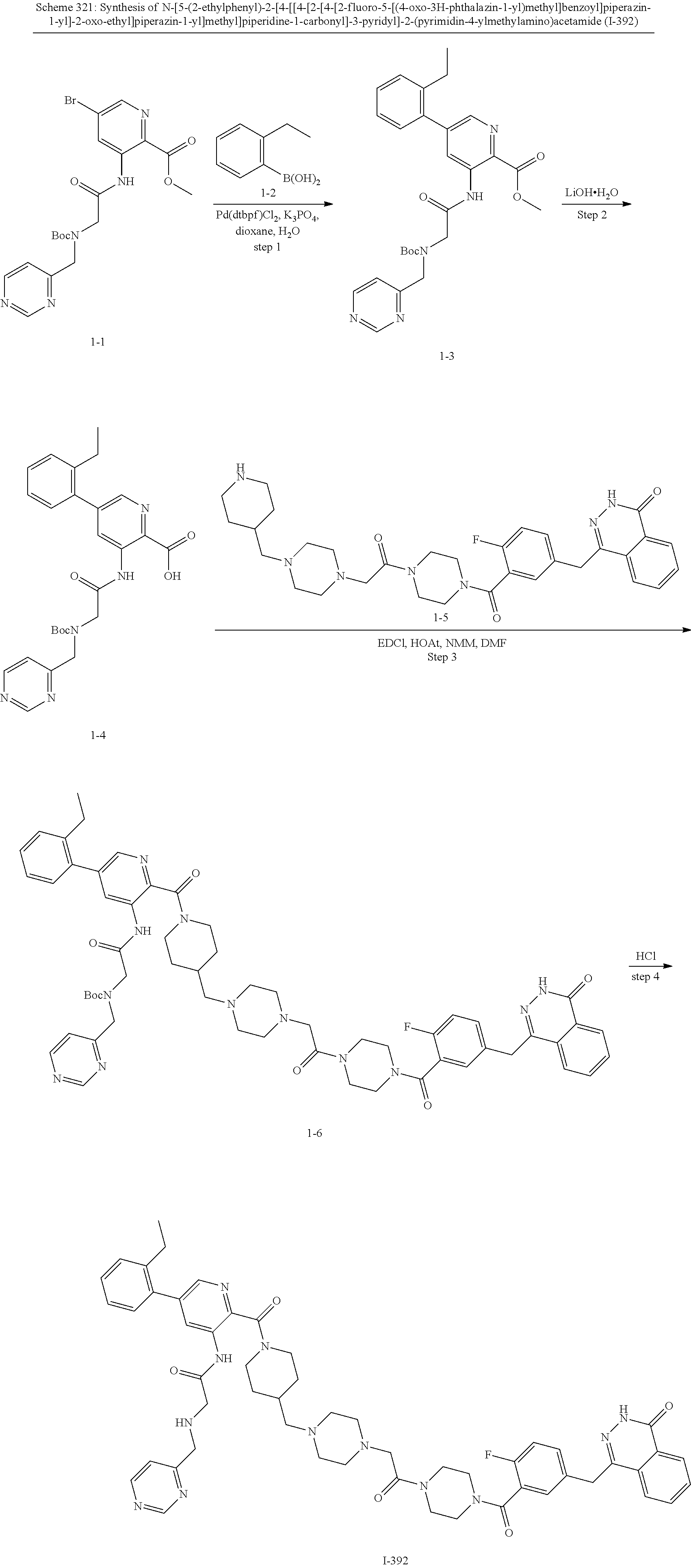

### Step 1: Synthesis of Intermediate 1-3.

To a mixture of Intermediate 1-1 (180 mg, 374.76 μmol, 1 eq) and Intermediate 1-2 (112.41 mg, 749.51 μmol, 2 eq) in dioxane (2 mL) and H₂O (0.4 mL) was added ditert-butyl(cyclopentyl)phosphane;dichloropalladium;iron (24.42 mg, 37.48 μmol, 0.1 eq) and K₃PO₄ (238.64 mg, 1.12 mmol, 3 eq), then the mixture was stirred at 80° C. for 1 hour under N₂. The reaction mixture was diluted with water (50 mL) and extracted with EA (10 mL*3). The combined organic layers were concentrated under reduced pressure to give a residue. The residue was purified by flash silica gel chromatography (ISCO; 20 g SepaFlash Silica Flash Column, Eluent of 0˜100% Ethylacetate/Petroleum ether gradient @60 mL/min). The eluent was concentrated under reduced pressure to give Intermediate 1-3 (120 mg, 237.36 μmol, 63.34% yield) as a white solid. LCMS (Method D): Retention time: 0.456 min, (M+H)=506.1.

### Step 2: Synthesis of Intermediate 1-4.

To a mixture of Intermediate 1-3 (120 mg, 237.36 μmol, 1 eq) in THF (2 mL), MeOH (1 mL), and H₂O (0.5 mL) was added LiOH·H₂O (29.88 mg, 712.08 μmol, 3 eq), then the mixture was stirred at 20° C. for 1 hour. The reaction mixture was concentrated under reduced pressure to remove THF, and then 1 N HCl was added (about 3 mL) to adjust the pH to 3˜4. The mixture was then extracted with EA (10 mL*3). The combined organic layers were concentrated under reduced pressure to give Intermediate 1-4 (60 mg, 122.07 μmol, 51.43% yield) as a white solid. LCMS (Method D): Retention time: 0.406 min, (M+H)=492.3.

### Step 3: Synthesis of Intermediate 1-6.

To a mixture of Intermediate 1-4 (40 mg, 81.38 μmol, 1 eq) and Intermediate 1-5 (47.99 mg, 81.38 μmol, 1 eq) in DMF (1 mL) was added HOAt (11.08 mg, 81.38 μmol, 11.38 μL, 1 eq), EDCI (46.80 mg, 244.13 μmol, 3 eq), NMM (41.16 mg, 406.89 μmol, 44.73 μL, 5 eq), then the mixture was stirred at 20° C. for 0.5 hour. The reaction mixture was diluted with water (50 mL) and extracted with EA (15 mL*2). The combined organic layers were concentrated under reduced pressure to give Intermediate 1-6 (80 mg, 75.24 μmol, 92.46% yield) as a white solid. LCMS (Method D): Retention time: 0.391 min, (M+H)=1063.8.

### Step 4: Synthesis of I-392

To a mixture of Intermediate 1-6 (50 mg, 47.03 μmol, 1 eq) in DCM (1 mL) was added HCl/dioxane (2 M, 117.57 μL, 5 eq), then the mixture was stirred at 20° C. for 0.5 hour. The reaction mixture was concentrated under reduced pressure to give a residue. The crude product was purified by reversed-phase HPLC (0.1% FA condition). The eluent was concentrated under reduced pressure to remove MeCN and then lyophilized to give I-392 (13.39 mg, 13.90 μmol, 29.56% yield, 100% purity) as a pink solid. LCMS (Method D): Retention time: 0.324 min, (M+H)=963.4. ¹H NMR (400 MHz, METHANOL-d4) δ=9.10 (s, 1H), 8.77 (d, J=4.8 Hz, 1H), 8.51 (s, 1H), 8.39-8.35 (m, 1H), 8.29 (s, 1H), 7.98-7.92 (m, 1H), 7.90-7.81 (m, 2H), 7.77 (d, J=3.2 Hz, 1H), 7.54-7.47 (m, 1H), 7.40-7.34 (m, 3H), 7.32-7.29 (m, 1H), 7.26-7.22 (m, 1H), 7.19-7.14 (m, 1H), 4.62 (d, J=12.8 Hz, 1H), 4.39 (s, 2H), 4.01 (s, 2H), 3.88-3.74 (m, 3H), 3.67 (d, J=1.6 Hz, 2H), 3.55-3.47 (m, 5H), 3.43-3.32 (m, 3H), 3.20-3.15 (m, 1H), 2.95-2.69 (m, 9H), 2.66-2.61 (m, 2H), 2.57-2.48 (m, 2H), 2.04-1.94 (m, 1H), 1.93-1.86 (m, 1H), 1.82-1.73 (m, 1H), 1.42-1.33 (m, 1H), 1.28-1.18 (m, 1H), 1.12 (t, J=7.6 Hz, 3H). ¹⁹F NMR (376 MHz, CHLOROFORM-d) δ=−120.79.

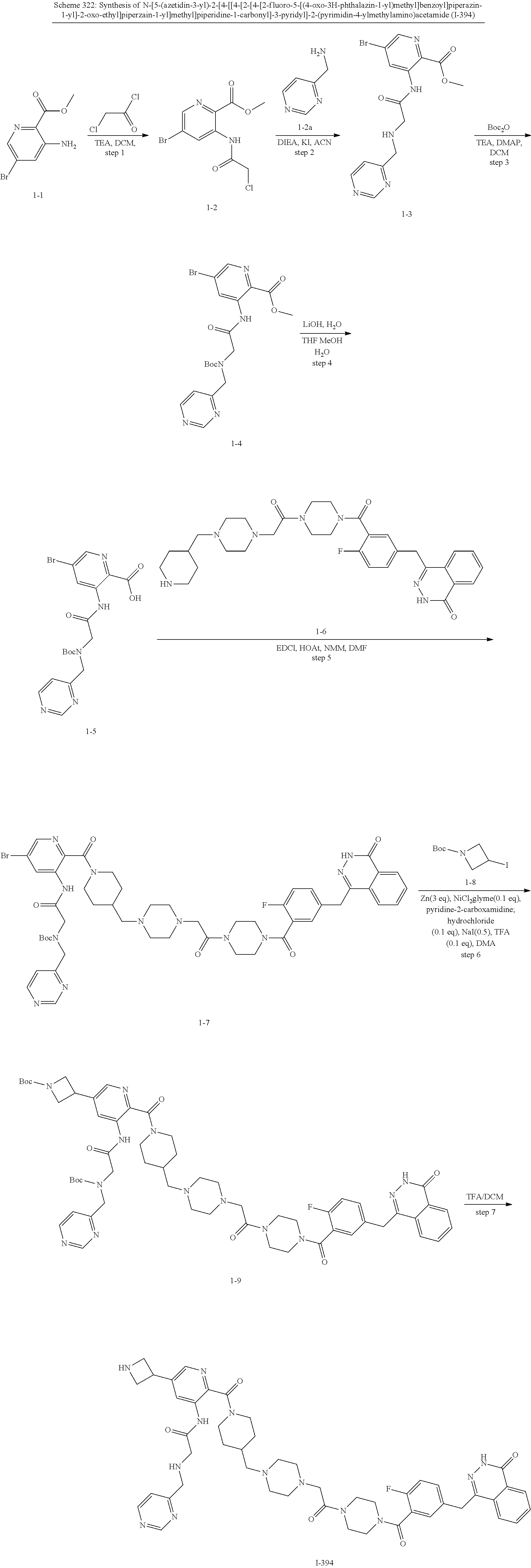

### Step 1: Synthesis of Intermediate 1-2

To a solution of Intermediate 1-1 (50 g, 216.41 mmol, 1 eq) in DCM (500 mL) was added TEA (43.80 g, 432.81 mmol, 60.24 mL, 2 eq) and 2-chloroacetyl chloride (36.66 g, 324.61 mmol, 25.85 mL, 1.5 eq). The mixture was stirred at 25° C. for 0.5 hr. The reaction mixture was quenched by addition of H₂O (50 mL) at 25° C., and then the reaction mixture was extracted with DCM (150 mL*2). The combined organic layers were washed with NaCl (aq) (50 mL*2), dried over Na₂SO₄, filtered and concentrated under reduced pressure to give a residue. The crude product was triturated with DCM at 25° C. for 30 min. The crude product was purified by prep-HPLC (column: Welch Ultimate XB—SiOH 250*50*10 um; mobile phase: [Hexane-EtOH]; gradient: 1%-18O % B over 15 min) and the eluent was concentrated to give the product. Intermediate 1-2 (70 g, crude) was obtained as a white solid. LCMS (Method D): Rt: 0.412 min, (M+H)=306.9. ¹H NMR (400 MHz, DMSO-d6) δ=8.86 (d, J=2.0 Hz, 1H), 8.59 (d, J=2.0 Hz, 1H), 4.48 (s, 2H), 3.89 (s, 3H).

### Step 2: Synthesis of Intermediate 1-3

To a solution of Intermediate 1-2 (56 g, 182.10 mmol, 1 eq) and Intermediate 1-2a (15.90 g, 145.68 mmol, 0.8 eq) in ACN (560 mL) was added DIEA (35.30 g, 273.15 mmol, 47.58 mL, 1.5 eq) and KI (60.46 g, 364.19 mmol, 2 eq). The mixture was stirred at 60° C. for 1 hr. The reaction mixture was diluted with H₂O (300 mL) and extracted with EA (500 mL*3). The combined organic layers were washed with NaCl(aq) (250 mL*2), dried over Na₂SO₄, filtered and concentrated under reduced pressure to give a residue. Intermediate 1-3 (70.5 g, crude) was obtained as a brown solid. LCMS (Method D): Rt: 0.245 min, (M+H)=381.7.

### Step 3: Synthesis of Intermediate 1-4

To a solution of Intermediate 1-3 (69 g, 181.49 mmol, 1 eq) in DCM (700 mL) was added DIEA (35.18 g, 272.23 mmol, 47.42 mL, 1.5 eq) and Boc₂O (47.53 g, 217.78 mmol, 50.03 mL, 1.2 eq) at 25° C., then the mixture was stirred at 25° C. for 0.5 hr. The mixture quenched with water (500 mL) and extracted with EA (500 mL*3), the combined organic phase was dried by Na₂SO₄, and concentrated under reduced pressure to give a residue. The residue was purified by flash silica gel chromatography (ISCO; 120 g SepaFlash Silica Flash Column, Eluent of 0˜100% EA/PE @100 mL/min, PE/EA=1:1, Rf=0.2) and the eluent was concentrated to give product. Intermediate 1-4 (60 g, 124.92 mmol, 68.83% yield) as yellow solid. LCMS (Method D): Rt: 0.426 min, (M+H)=480.0.

### Step 4: Synthesis of Intermediate 1-5

To a solution of Intermediate 1-4 (3 g, 6.25 mmol, 1 eq) in THF (10 mL), H₂O (10 mL), and MeOH (10 mL) was added LiOH·H₂O (786.31 mg, 18.74 mmol, 3 eq). The mixture was stirred at 25° C. for 1 hr. The reaction mixture was adjusted to pH=6 by HCl (2M), and then extracted with DCM (20 mL*3). The combined organic layers were washed with NaCl (aq) (10 mL*2), dried over Na₂SO₄, filtered and concentrated under reduced pressure to give a residue. Intermediate 1-5 (1.85 g, 3.97 mmol, 63.52% yield) was obtained as a yellow oil. LCMS (Method D): Rt: 0.400 min, (M+H)=466.8. ¹H NMR (400 MHz, CHLOROFORM-d) δ=11.57-11.37 (m, 1H), 9.47 (s, 1H), 9.16 (s, 1H), 8.74 (d, J=4.4 Hz, 1H), 8.36 (s, 1H), 4.72-4.64 (m, 2H), 4.33-4.19 (m, 2H), 1.44 (s, 9H).

### Step 5: Synthesis of Intermediate 1-7

To a solution of Intermediate 1-5 (1.8 g, 3.86 mmol, 1 eq) and Intermediate 1-6 (2.42 g, 3.86 mmol, 1 eq, HCl salt) in DMF (18 mL) was added EDCI (2.22 g, 11.58 mmol, 3 eq) and HOAt (525.43 mg, 3.86 mmol, 540.01 μL, 1 eq), and NMM (1.95 g, 19.30 mmol, 2.12 mL, 5 eq). The mixture was stirred at 25° C. for 0.5 hr. The reaction mixture was concentrated under reduced pressure to give a residue. The residue was purified by reversed phase HPLC (0.1% FA) the eluent was concentrated to remove ACN and lyophilized to give the product. Intermediate 1-7 (1.4 g, 1.22 mmol, 31.48% yield, TFA salt) was obtained as a yellow solid. LCMS (Method D): Rt: 0.383 min, (M+H)=1037.4. ¹H NMR (400 MHz, CHLOROFORM-d) δ=10.94-10.60 (m, 1H), 10.49-10.33 (m, 1H), 9.21-9.09 (m, 1H), 8.93 (s, 1H), 8.72 (s, 1H), 8.51-8.41 (m, 1H), 8.35 (d, J=2.0 Hz, 1H), 8.14 (s, 2H), 7.83-7.78 (m, 2H), 7.76 (d, J=9.6 Hz, 1H), 7.39-7.29 (m, 3H), 7.09-7.04 (m, 1H), 4.72-4.58 (m, 3H), 4.30 (s, 2H), 4.22 (s, 1H), 4.11 (s, 2H), 3.80 (d, J=1.6 Hz, 3H), 3.62-3.51 (m, 4H), 3.49-3.26 (m, 8H), 3.08 (d, J=11.6 Hz, 3H), 2.99 (s, 2H), 2.75 (s, 2H), 2.17-1.90 (m, 3H), 1.45-1.38 (m, 9H). ¹⁹F NMR (400 MHz, METHAN OL-d4) δ=−75.761, −117.559.

### Step 6: Synthesis of Intermediate 1-9

To a solution of Intermediate 1-7 (439.97 mg, 423.87 μmol, 1 eq) and Intermediate 1-8 (120 mg, 423.87 μmol, 1 eq) in DMA (5 mL) was added Zn (55.43 mg, 847.74 μmol, 2 eq), pyridine-2-carboximidamide hydrochloride (6.68 mg, 42.39 μmol, 0.1 eq), NaI (31.77 mg, 211.93 μmol, 0.5 eq), TFA (4.83 mg, 42.39 μmol, 3.15 μL, 0.1 eq) and NiCl₂-glyme (9.31 mg, 42.39 μmol, 0.1 eq). The suspension was degassed and purged with N₂ 3 times. The mixture was stirred at 60° C. for 2 hr under N₂. The reaction mixture was filtered and diluted with H₂O (12 mL) and then extracted with EA (4 mL*3). The combined organic layers were dried over sodium sulfate, filtered and concentrated under reduced pressure to give a residue. The solid was treated with 10 mL of 1M HCl. The crude product was purified by reversed-phase HPLC (0.1% FA condition). The eluent was concentrated under reduced pressure to remove ACN and then lyophilized to give Intermediate 1-9 (60 mg, 50.01 μmol, 11.80% yield, 92.88% purity) as a brown solid. LCMS (Method D): Rt: 0.396 min, (M+H)=1114.5.

### Step 7: Synthesis of I-394

To a solution of Intermediate 1-9 (55 mg, 49.36 μmol, 1 eq) in DCM (1 mL) was added TFA (100 L). The mixture was stirred at 25° C. for 1 hr. The mixture was concentrated to give a residue. The residue was purified by prep-HPLC (column: CD04-Welch Utimate C18 150*25*7 um; mobile phase: [water (TFA)-ACN]; gradient: 2%-32% B over 10 min). The eluent was concentrated under reduced pressure to remove ACN and then lyophilized to give I-394 (27.52 mg, 26.77 μmol, 54.23% yield, 100% purity, TFA salt) as a yellow solid. LCMS (Method D): Rt: 0239 min, (M+H)=914.3. ¹H NMR (400 MHz, METHANOL-d4) δ=9.24 (s, 1H), 8.84 (d, J=5.2 Hz, 1H), 8.49-8.42 (m, 1H), 8.40-8.33 (m, 1H), 8.21 (d, J=2.0 Hz, 1H), 7.99-7.92 (m, 1H), 7.90-7.80 (m, 2H), 7.60 (d, J=5.2 Hz, 1H), 7.55-7.46 (m, 1H), 7.40-7.31 (m, 1H), 7.22-7.11 (m, 1H), 4.67-4.55 (m, 3H), 4.48-4.30 (m, 8H), 4.23 (s, 2H), 3.95-3.78 (m, 4H), 3.78-3.67 (m, 3H), 3.60 (s, 1H), 3.53 (d, J=4.4 Hz, 1H), 3.44 (s, 1H), 3.35 (s, 4H), 3.21 (s, 4H), 3.18-3.13 (m, 1H), 3.00-2.87 (m, 3H), 2.16 (s, 1H), 1.95 (d, J=12.4 Hz, 1H), 1.76 (d, J=12.4 Hz, 1H), 1.61-1.47 (m, 1H), 1.45-1.31 (m, 1H). ¹⁹F NMR (400 MHz, METHAN OL-d4) δ=−77.074, −120.710.

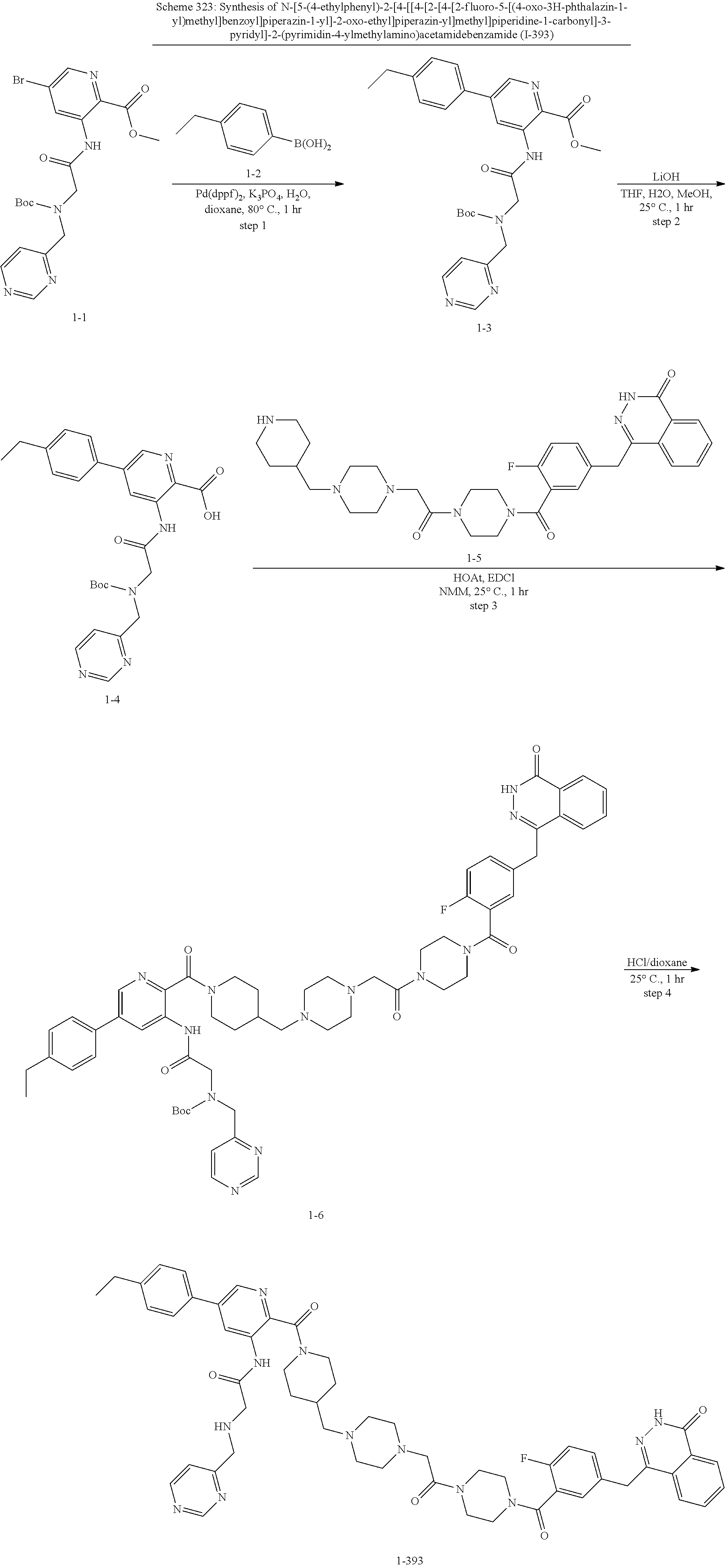

### Step 1: Synthesis of Intermediate 1-3

To a solution of intermediate 1-2 (150 mg, 312.30 μmol, 1 eq) and intermediate 1-2 (56.21 mg, 374.76 μmol, 1.2 eq) in dioxane (1.5 mL) and H₂O (0.3 mL) was added ditert-butyl(cyclopentyl)phosphane;dichloropalladium;iron (20.35 mg, 31.23 μmol, 0.1 eq) and K₃PO₄ (198.87 mg, 936.89 μmol, 3 eq), and the mixture was degassed and purged with N₂ 3 times, and then the mixture was stirred at 80° C. for 1 hr under N₂ atmosphere. The reaction mixture was concentrated under reduced pressure to give a residue. The residue was purified by flash silica gel chromatography (ISCO; 12 g SepaFlash Silica Flash Column, Eluent of 0˜100% Ethyl acetate/Petroleum ether gradient @40 mL/min) and concentrated under reduced pressure to give a residue. Intermediate 1-3 (170 mg, crude) was obtained. LCMS (Method D): Rt=0.478 min, (M+H)=506.2.

### Step 2: Synthesis of Intermediate 1-4

To a mixture of intermediate 1-3 (120 mg, 237.36 μmol, 1 eq) in MeOH (0.5 mL), THF (0.5 mL) and H₂O (0.5 mL) was added LiOH·H₂O (49.80 mg, 1.19 mmol, 5 eq). The mixture was stirred at 25° C. for 1 hr. The reaction mixture was adjusted to pH=7 with HCl (1 M) and extracted with EA (2 mL*3), the combined organic phase was dried by Na₂SO₄, filtered and concentrated under reduced pressure to give a residue. Intermediate 1-4 (165 mg, crude) was obtained as a brown solid. LCMS (Method D): Rt=0.417 min, (M+H)=492.1.

### Step 3: Synthesis of Intermediate 1-6

To a solution of intermediate 1-4 (70 mg, 142.41 μmol, 1 eq) and intermediate 1-5 (98.09 mg, 156.65 μmol, 1.1 eq, HCl salt) in DMF (1 mL) was added EDCI (81.90 mg, 427.23 μmol, 3 eq), HOAt (19.38 mg, 142.41 μmol, 19.92 μL, 1 eq) and NMM (72.02 mg, 712.05 μmol, 78.28 μL, 5 eq). The mixture was stirred at 25° C. for 1 hr. The reaction mixture was concentrated under reduced pressure to give a residue. The crude product was purified by reversed-phase HPLC (0.1% FA condition) followed by lyophilization to give a residue. Intermediate 1-6 (30 mg, 28.22 μmol, 19.81% yield, 100% purity) was obtained as a white solid. LCMS (Method D): Rt=0.442 min, (M+H)=1064.0.

### Step 4: Synthesis of I-393

To a solution of Intermediate 1-6 (30 mg, 28.22 μmol, 1 eq) was added HCl/dioxane (2 M, 0.5 mL, 35.44 eq). The mixture was stirred at 25° C. for 1 hr. The reaction mixture was concentrated under reduced pressure to give a residue. To the residue was added ACN and H₂O, followed by lyophilization to give product, which was used without further purification. I-393 (26.35 mg, 26.10 μmol, 92.52% yield, 99.025% purity, HCl salt) was obtained as a yellow solid. LCMS (Method F): Rt=0.360 min, (M+H)=963.5. ¹H NMR (400 MHz, METHANOL-d4) δ=9.30 (s, 1H), 8.88 (d, J=5.2 Hz, 1H), 8.76 (s, 1H), 8.48 (s, 1H), 8.37 (d, J=7.6 Hz, 1H), 7.99-7.92 (m, 1H), 7.92-7.80 (m, 2H), 7.72-7.61 (m, 3H), 7.51 (s, 1H), 7.42-7.35 (m, 3H), 7.17 (t, J=9.2 Hz, 1H), 4.65 (s, 3H), 4.57 (d, J=17.2 Hz, 2H), 4.39 (s, 2H), 4.30 (s, 2H), 3.92 (s, 5H), 3.88-3.76 (m, 5H), 3.76-3.65 (m, 2H), 3.56 (d, J=4.4 Hz, 2H), 3.41 (s, 2H), 3.34 (d, J=6.4 Hz, 3H), 3.27 (s, 1H), 3.02 (t, J=12.8 Hz, 1H), 2.77-2.69 (m, 2H), 2.40-2.26 (m, 1H), 2.09 (d, J=11.6 Hz, 1H), 1.93 (d, J=11.2 Hz, 1H), 1.68-1.58 (m, 1H), 1.58-1.47 (m, 1H), 1.28 (t, J=7.6 Hz, 3H).

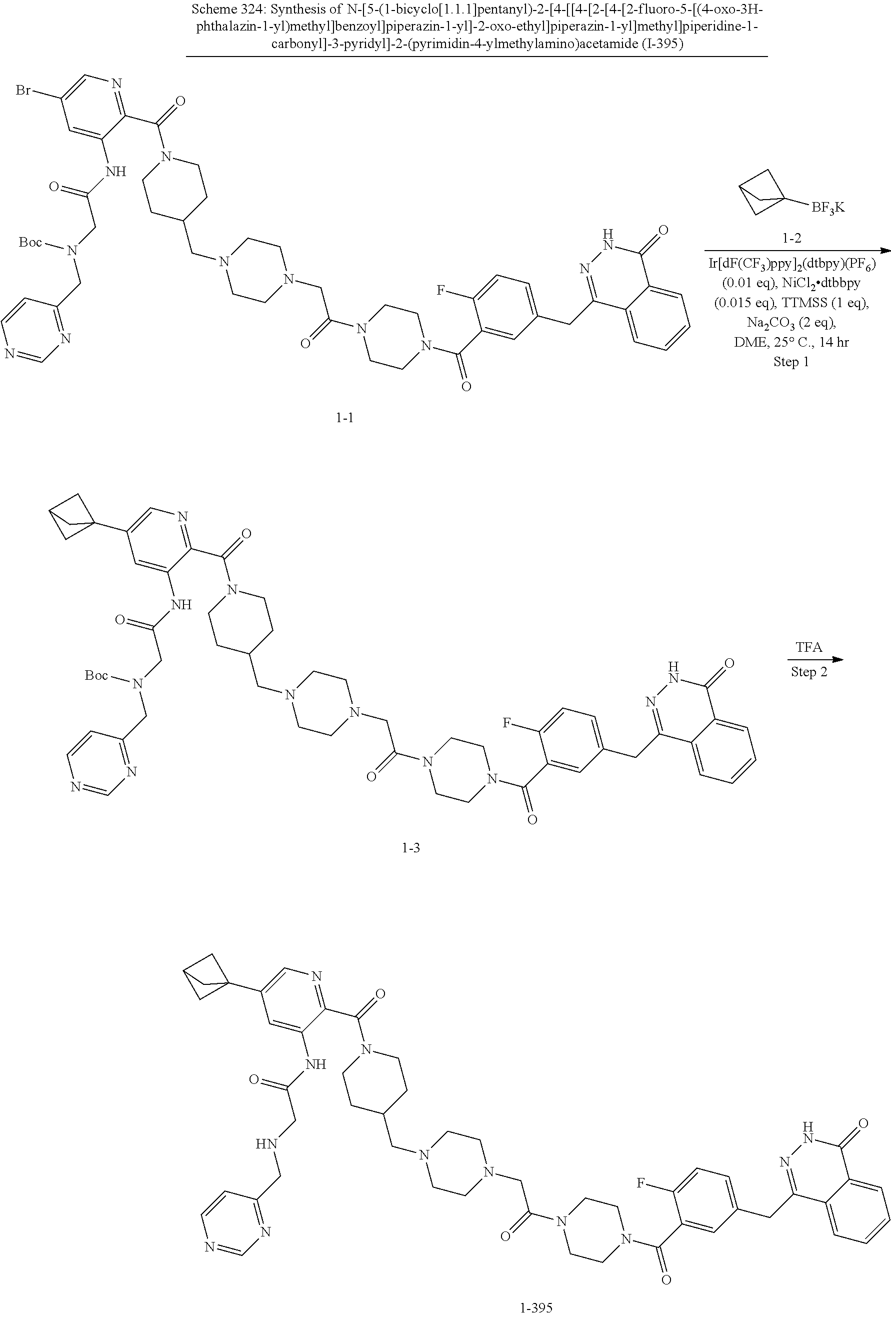

### Step 1: Synthesis of Intermediate 1-3

To a solution of Intermediate 1-2 (125.74 mg, 722.56 μmol, 1.5 eq) and Intermediate 1-1 (500 mg, 481.71 μmol, 1 eq) in DMA (10 mL) was added Ir[dF(CF₃)ppy]₂(bpy)(PF₆) (48.64 mg, 48.17 μmol, 0.1 eq), Na₂CO₃ (102.11 mg, 963.42 μmol, 2 eq) and NiCl₂-dtbpy (28.76 mg, 72.26 μmol, 0.15 eq). The vial was sealed and placed under nitrogen. The reaction was stirred and irradiated with a blue 10 W LED lamp (3 cm away), with cooling water to keep the reaction temperature at 25° C. for 14 hr. The reaction mixture was diluted with water (120 mL) and extracted with ethyl acetate (40 mL×3). The combined organic layers were washed with brine (40 mL×3) and dried over sodium sulfate, filtered and concentrated under reduced pressure to give a residue. The crude product was purified by reversed-phase HPLC (0.1% FA condition). The eluent was concentrated under reduced pressure to remove ACN and then lyophilized to give Intermediate 1-3 (160 mg, 155.02 μmol, 32.18% yield, 99.33% purity) as a brown solid. LCMS (Method D): Rt: 0.383 min, (M+H)=1025.5.

### Step 2: Synthesis of I-395

To a solution of Intermediate 1-3 (140 mg, 136.56 μmol, 1 eq) in DCM (1 mL) was added TFA (200 L). The mixture was stirred at 25° C. for 1 hr. The mixture was concentrated to give a residue. The crude product was purified by reversed-phase HPLC (or 0.1% TFA condition). The eluent was concentrated under reduced pressure to remove ACN and then lyophilized to give I-395 (70.86 mg, 67.51 μmol, 49.44% yield, 99% purity, TFA salt) as a yellow solid. LCMS (Method D): Rt: 0.303 min, (M+H)=925.4. ¹H NMR (400 MHz, METHANOL-d4) δ=9.25 (s, 1H), 8.84 (d, J=5.2 Hz, 1H), 8.40-8.34 (m, 1H), 8.27 (d, J=1.6 Hz, 1H), 7.99-7.91 (m, 2H), 7.91-7.79 (m, 2H), 7.59 (d, J=5.2 Hz, 1H), 7.54-7.46 (m, 1H), 7.45-7.37 (m, 1H), 7.21-7.12 (m, 1H), 4.66-4.55 (m, 3H), 4.38 (s, 2H), 4.19 (s, 2H), 4.05 (s, 1H), 3.98 (s, 1H), 3.84-3.74 (m, 2H), 3.70 (d, J=5.6 Hz, 2H), 3.60-3.52 (m, 2H), 3.52-3.32 (m, 11H), 3.20-3.11 (m, 1H), 3.10-2.98 (m, 2H), 2.96-2.84 (m, 1H), 2.61 (s, 1H), 2.26-2.11 (m, 7H), 1.99-1.90 (m, 1H), 1.75 (d, J=12.8 Hz, 1H), 1.61-1.46 (m, 1H), 1.46-1.32 (m, 1H). ¹⁹F NMR (400 MHz, METHAN OL-d4) δ=−77.384, −120.710.

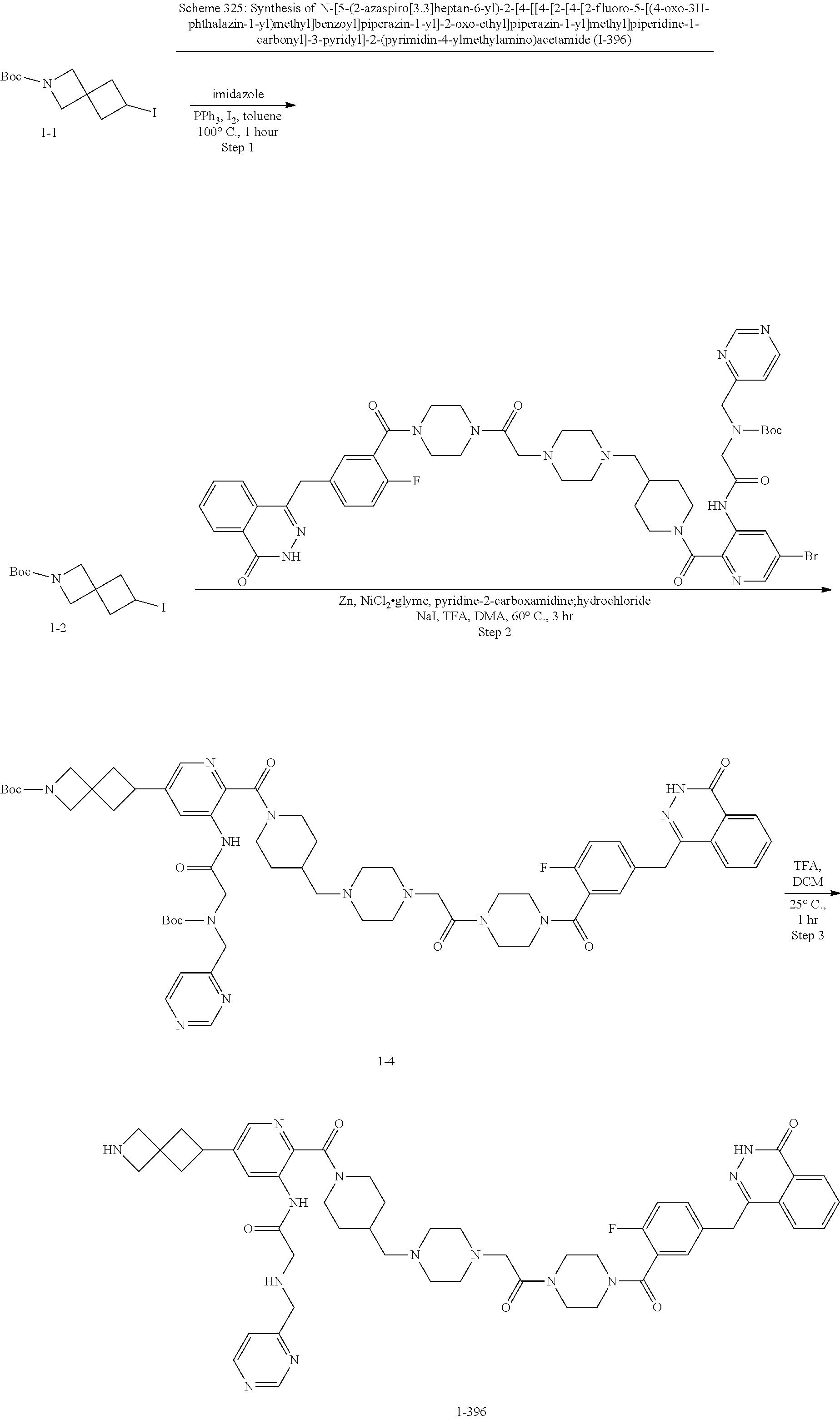 [SMILES: CC(C)(C)OC(=O)N1CC2(CC(I)C2)C1 ; CC(C)(C)OC(=O)N1CC2(CC(I)C2)C1 ; CC(C)(C)OC(=O)N(CC(=O)Nc1cc(Br)cnc1C(=O)N1CCC(CN2CCN(CC(=O)N3CCN(C(=O)c4cc(Cc5n[nH]c(=O)c6ccccc56)ccc4F)CC3)CC2)CC1)Cc1ccncn1 ; CC(C)(C)OC(=O)N(CC(=O)Nc1cc(C2CC3(C2)CN(C(=O)OC(C)(C)C)C3)cnc1C(=O)N1CCC(CN2CCN(CC(=O)N3CCN(C(=O)c4cc(Cc5n[nH]c(=O)c6ccccc56)ccc4F)CC3)CC2)CC1)Cc1ccncn1 ; O=C(CNCc1ccncn1)Nc1cc(C2CC3(CNC3)C2)cnc1C(=O)N1CCC(CN2CCN(CC(=O)N3CCN(C(=O)c4cc(Cc5n[nH]c(=O)c6ccccc56)ccc4F)CC3)CC2)CC1]

### Step 1: Synthesis of Intermediate 1-2

To a solution of Intermediate 1-1 (500 mg, 2.34 mmol, 1 eq) in toluene (30 mL) was added imidazole (478.80 mg, 7.03 mmol, 3 eq) and iodine (892.55 mg, 3.52 mmol, 708.37 μL, 1.5 eq), triphenylphosphine (1.23 g, 4.69 mmol, 2 eq), and then the mixture was stirred at 100° C. for 1 hour. The reaction mixture was diluted with water (80 mL) and extracted with EA (30 mL*2). The combined organic layers were concentrated under reduced pressure to give a residue. The residue was purified by flash silica gel chromatography (ISCO; 20 g SepaFlash Silica Flash Column, Eluent of 0˜30% Ethyl acetate/Petroleum ether gradient @80 mL/min). The eluent was concentrated under reduced pressure to give Intermediate 1-2 (600 mg, 1.86 mmol, 79.19% yield) as a white solid. ¹H NMR (400 MHz, DMSO-d₆) δ=4.47-4.39 (m, 1H), 3.85 (s, 4H), 2.95-2.83 (m, 2H), 2.70-2.58 (m, 2H), 1.36 (s, 9H).

### Step 2: Synthesis of Intermediate 1-4

To a solution of Intermediate 1-3 (200 mg, 192.68 μmol, 1 eq) and Intermediate 1-2 (62.27 mg, 192.68 μmol, 1 eq) in DMA (2 mL) was added pyridine-2-carboxamidine; hydrochloride (3.04 mg, 19.27 μmol, 0.1 eq), NaI (14.44 mg, 96.34 μmol, 0.5 eq), TFA (2.20 mg, 19.27 μmol, 1.43 μL, 0.1 eq), dichloronickel;1,2-dimethoxyethane (4.23 mg, 19.27 μmol, 0.1 eq) and Zn (25.20 mg, 385.37 μmol, 2 eq). The suspension was degassed and purged with N₂ 3 times. The mixture was stirred at 60° C. for 2 hours under N₂. The crude product was combined with another lot of material for workup. The reaction mixture was filtered and diluted with H₂O (4 mL) and then extracted with EA (3 mL*2). The combined organic layers were dried over Na₂SO₄, filtered and concentrated under reduced pressure to give a residue. The solid was treated with 10 mL of 1M HCl. The crude product was purified by reversed-phase HPLC (0.1% FA condition). The eluent was concentrated under reduced pressure to remove ACN and then lyophilized to give Intermediate 1-4 (50 mg, 43.32 μmol, 22.48% yield) as a white solid. LCMS (Method D): Rt: 0.397 min, (M+H)=1154.8.

### Step 3: Synthesis of I-396

A solution of Intermediate 1-4 (45 mg, 38.98 μmol, 1 eq) in CH₂Cl₂ (0.5 mL) and TFA (0.1 mL) was stirred at 25° C. for 2 hours. The mixture was concentrated under vacuum. The crude product was purified by reversed-phase HPLC (0.1% TFA condition). The eluent was concentrated under reduced pressure to remove ACN and then lyophilized to give I-396 (25.87 mg, 24.22 μmol, 62.13% yield, 100% purity, TFA salt) as a yellow solid. LCMS (Method D): Rt: 0.244 min, (M+H)=954.8. ¹H NMR (400 MHz, METHANOL-d₄) δ=9.24 (s, 1H), 8.87-8.81 (m, 1H), 8.40-8.34 (m, 1H), 8.31-8.26 (m, 1H), 8.01-7.92 (m, 2H), 7.90-7.81 (m, 2H), 7.59 (d, J=4.4 Hz, 1H), 7.55-7.47 (m, 1H), 7.41-7.32 (m, 1H), 7.21-7.13 (m, 1H), 4.65-4.59 (m, 1H), 4.57 (s, 2H), 4.39 (s, 2H), 4.28 (s, 2H), 4.20 (s, 2H), 4.05 (s, 2H), 3.84-3.66 (m, 7H), 3.65-3.50 (m, 4H), 3.45 (s, 1H), 3.38-3.34 (m, 1H), 3.28-3.22 (m, 3H), 3.17-3.07 (m, 5H), 2.95-2.87 (m, 3H), 2.81-2.73 (m, 2H), 2.51-2.41 (m, 2H), 2.19-2.07 (m, 1H), 1.98-1.89 (m, 1H), 1.79-1.69 (m, 1H), 1.56-1.43 (m, 1H), 1.42-1.30 (m, 1H). ¹⁹F NMR (376 MHz, MeOD-d6) δ=−77.097, −120.726.

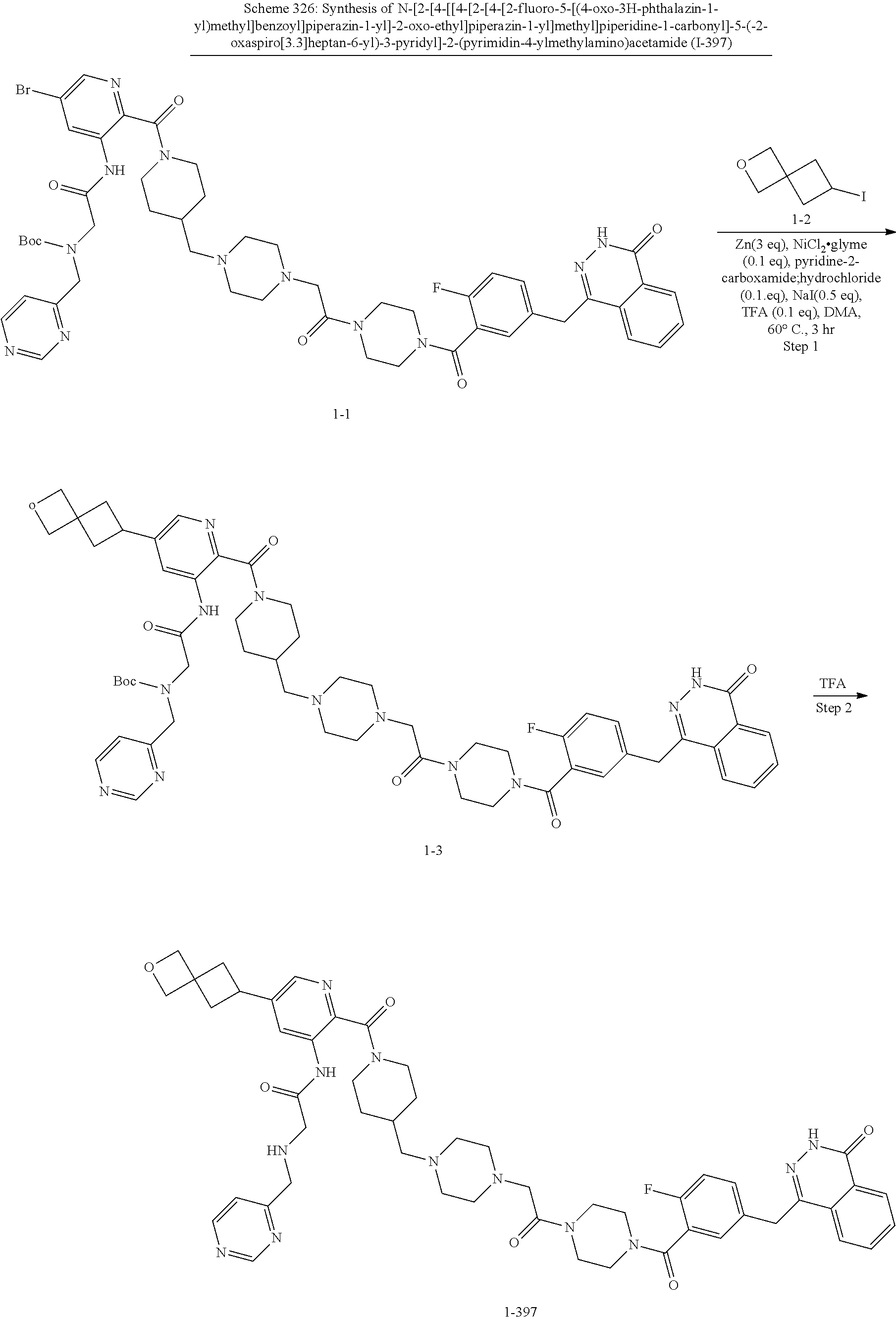

### Step 1: Synthesis of Intermediate 1-3

To a solution of Intermediate 1-1 (200 mg, 192.68 μmol, 1 eq) and Intermediate 1-2 (43.17 mg, 192.68 μmol, 1 eq) in DMA (2 mL) was added Zn (25.20 mg, 385.37 μmol, 2 eq), pyridine-2-carboximidamide hydrochloride (3.04 mg, 19.27 μmol, 0.1 eq), NaI (14.44 mg, 96.34 μmol, 0.5 eq), TFA (2.20 mg, 19.27 μmol, 1.43 μL, 0.1 eq) and NiCl₂-glyme (4.23 mg, 19.27 μmol, 0.1 eq). The suspension was degassed and purged with N₂ 3 times. The mixture was stirred at 60° C. for 3 hr. The reaction mixture was filtered and diluted with H₂O (15 mL) and then extracted with EA (5 mL*3). The combined organic layers were dried over sodium sulfate, filtered and concentrated under reduced pressure to give a residue. The solid was treated with 10 mL of 1M HCl. The crude product was purified by reversed-phase HPLC (0.1% FA condition). The eluent was concentrated under reduced pressure to remove ACN and then lyophilized to give Intermediate 1-3 (160 mg, 131.72 μmol, 68.36% yield, 86.87% purity) as a brown solid. LCMS (Method D): Rt: 0.351 min, (M+H)=1055.4.

### Step 2: Synthesis of I-397

To a solution of Intermediate 1-3 (140 mg, 132.68 μmol, 1 eq) in DCM (1 mL) was added TFA (307.00 mg, 2.69 mmol, 200 μL, 20.29 eq). The mixture was stirred at 25° C. for 1 hr. The reaction mixture was diluted with water (21 mL) and extracted with ethyl acetate (7 mL×3). The combined organic layers were washed with brine (7 mL×3) and dried over sodium sulfate, filtered and concentrated under reduced pressure to give a residue. The residue was purified by prep-HPLC (column: CD07-Daisogel SP-100-8-ODS-PK 150*25*10 um; mobile phase: [water (NH₄HCO₃)-ACN]; gradient: 10%-40% B over 10 min). The eluent was concentrated under reduced pressure to remove ACN and then lyophilized to give I-397 (21.75 mg, 21.87 μmol, 16.49% yield, 96.053% purity) as a yellow solid. LCMS (Method F): Rt: 0.499 min, (M+H)=955.7. ¹H NMR (400 MHz, METHANOL-d4) δ=9.09 (s, 1H), 8.75 (d, J=5.2 Hz, 1H), 8.43 (s, 1H), 8.36 (d, J=7.6 Hz, 1H), 8.18 (d, J=1.2 Hz, 1H), 7.98-7.91 (m, 1H), 7.90-7.79 (m, 2H), 7.76 (d, J=5.2 Hz, 1H), 7.48 (s, 1H), 7.43-7.33 (m, 1H), 7.22-7.10 (m, 1H), 4.69-4.48 (m, 4H), 4.38 (s, 2H), 3.98 (s, 2H), 3.82-3.62 (m, 6H), 3.59-3.40 (m, 6H), 3.36-3.32 (m, 1H), 3.26 (s, 1H), 3.20 (s, 1H), 3.14-3.02 (m, 1H), 2.85-2.71 (m, 3H), 2.60-2.43 (m, 7H), 2.42-2.34 (m, 3H), 2.24-2.15 (m, 2H), 1.83 (s, 2H), 1.70 (d, J=12.0 Hz, 1H), 1.24-1.05 (m, 2H). ¹⁹F NMR (400 MHz, METHAN OL-d4) δ=−120.710.

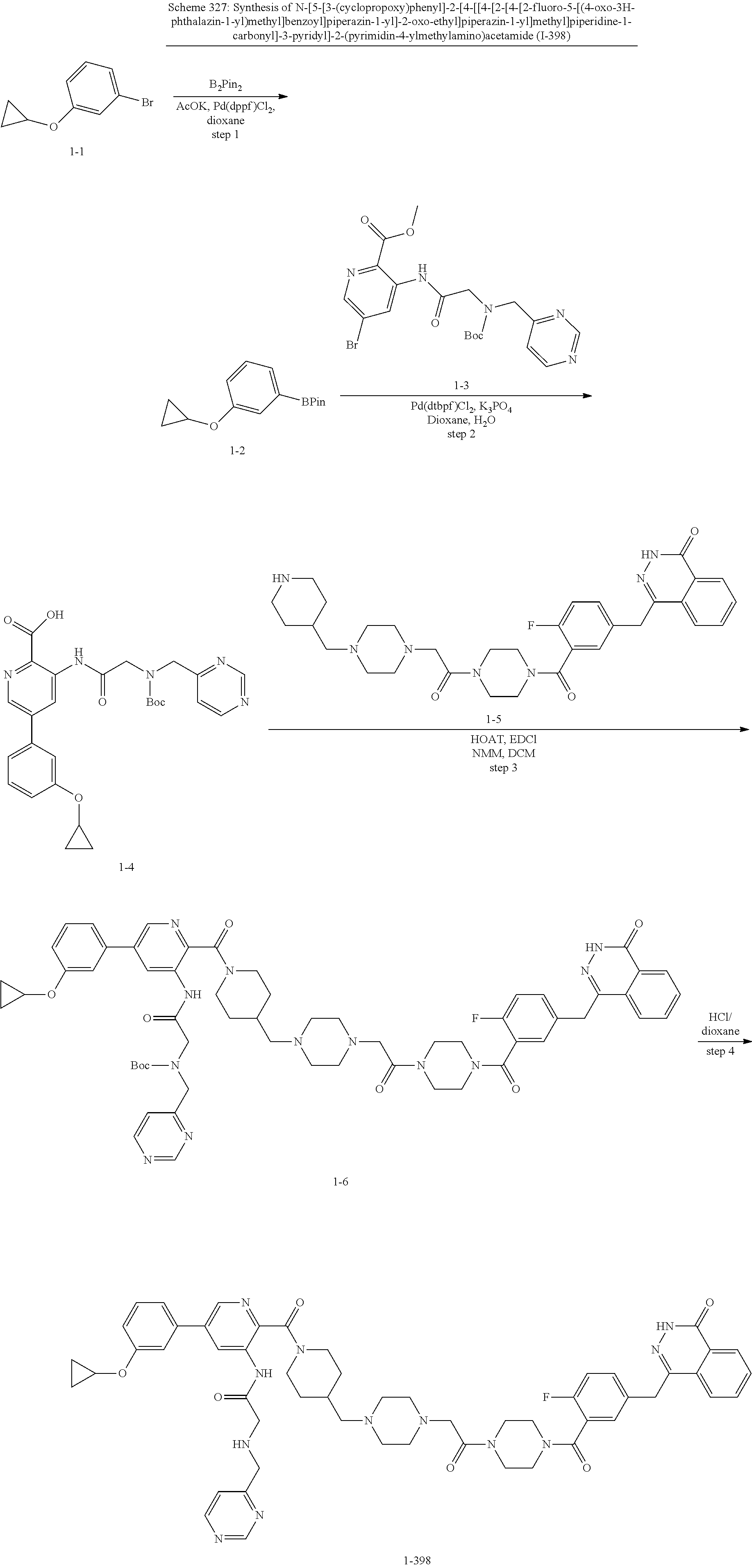

### Step 1: Synthesis of Intermediate 1-2.

A mixture of Intermediate 1-1 (500 mg, 2.35 mmol, 1 eq) and Pin₂B₂ (715.08 mg, 2.82 mmol, 1.2 eq) in dioxane (5 mL) was added potassium; acetate (690.91 mg, 7.04 mmol, 3 eq) and Pd(dppf)Cl₂ (171.70 mg, 234.66 μmol, 0.1 eq), and then the mixture was purged with N₂ 3 times. The mixture was stirred at 80° C. for 1 hr under N₂ atmosphere. The reaction mixture was diluted with H₂O (6 mL) and extracted with EA (3 mL*3). The combined organic layers were dried over Na₂SO₄, filtered and concentrated under reduced pressure to give a residue. The residue was purified by flash silica gel chromatography (ISCO; 20 g SepaFlash Silica Flash Column, Eluent of 10% EA/PE gradient @60 mL/min). The eluent concentrated under reduced pressure to give Intermediate 1-2 (500 mg, 1.74 mmol, 74.17% yield, 90.558% purity) as yellow oil. LCMS (Method D): Retention time: 0.502 min, (M+H)=261.1. ¹H NMR (400 MHz, CHLOROFORM-d) δ=7.39 (d, J=2 Hz, 1H), 7.32 (d, J=7.2 Hz, 1H), 7.22-7.15 (m, 1H), 7.02 (m, 1H), 3.68 (m, 1H), 1.25 (s, 12H), 0.70-0.64 (m, 4H).

### Step 2: Synthesis of Intermediate 1-4.

A mixture of Intermediate 1-2 (450 mg, 1.73 mmol, 1 eq), Intermediate 1-3 (830.88 mg, 1.73 mmol, 1 eq), K₃PO₄ (1.10 g, 5.19 mmol, 3 eq) and Pd(dtbpf)Cl₂ (112.74 mg, 172.99 μmol, 0.1 eq) in dioxane (1.2 mL) and H₂O (0.12 mL) was degassed and purged with N₂ 3 times. The mixture was stirred at 80° C. for 1 hr under N₂ atmosphere. The reaction mixture was diluted with H₂O (6 mL) and extracted with EA (6 mL*3). The combined organic layers were dried over Na₂SO₄, filtered and concentrated under reduced pressure to give a residue. The residue was purified by flash silica gel chromatography (ISCO; 20 g SepaFlash Silica Flash Column, Eluent of 10% MeOH/EA gradient @60 mL/min). The eluent concentrated under reduced pressure to give Intermediate 1-4 (340 mg, 506.90 μmol, 29.30% yield, 77.459% purity) as a white solid. LCMS (Method D): Retention time: 0.430 min, (M+H)=520.2. ¹H NMR (400 MHz, DMSO-d6) δ=¹H NMR (400 MHz, METHANOL-d4) δ=9.52-8.27 (m, 5H), 7.62-7.05 (m, 4H), 4.32-4.07 (m, 2H), 3.35 (s, 4H), 1.36-1.25 (m, 9H), 0.86-0.58 (m, 4H).

### Step 3: Synthesis of Intermediate 1-6.

A mixture of Intermediate 1-5 (215.66 mg, 365.70 μmol, 1 eq) and Intermediate 1-4 (190 mg, 365.70 μmol, 1 eq) in DMF (10 mL) was added HOAt (49.78 mg, 365.70 μmol, 51.16 μL, 1 eq), EDCI (210.32 mg, 1.10 mmol, 3 eq), and NMM (184.95 mg, 1.83 mmol, 201.04 μL, 5 eq). The mixture was stirred at 40° C. for 1 hr. The reaction mixture was diluted with H₂O (6 mL) and extracted with EA (6 mL*3). The combined organic layers were washed with brine (5 mL*3), dried over Na₂SO₄, filtered and concentrated under reduced pressure to give a residue. The residue was purified by prep-HPLC (FA condition). The eluent was concentrated and lyophilized to give Intermediate 1-6 (30 mg, 26.16 μmol, 7.15% yield, 95.155% purity) as a red solid. LCMS (Method D): Retention time: 0.421 min, (M+H)=546.5. ¹H NMR (400 MHz, CHLOROFORM-d) δ=¹H NMR (400 MHz, METHANOL-d4) δ=9.17-9.04 (m, 1H), 8.77-8.63 (m, 2H), 8.40-8.32 (m, 2H), 8.00-7.93 (m, 1H), 7.90-7.80 (m, 2H), 7.56 (d, J=4.0 Hz, 1H), 7.49 (d, J=3.6 Hz, 1H), 7.46-7.40 (m, 1H), 7.40-7.33 (m, 2H), 7.28 (d, J=7.6 Hz, 1H), 7.19-7.13 (m, 2H), 4.85-4.82 (m, 1H), 4.69 (s, 2H), 4.64-4.57 (m, 1H), 4.38 (s, 2H), 4.30-4.20 (m, 2H), 3.90-3.86 (m, 1H), 3.81-3.64 (m, 5H), 3.58-3.46 (m, 2H), 3.41-3.33 (m, 2H), 3.21-3.13 (m, 1H), 2.91-2.81 (m, 1H), 2.79-2.53 (m, 8H), 2.47-2.35 (m, 2H), 1.90 (d, J=11.6 Hz, 2H), 1.78-1.66 (m, 1H), 1.46 (s, 3H), 1.31 (s, 9H), 0.85-0.80 (m, 2H), 0.73 (s, 2H). ¹⁹F NMR (376 MHz, CHLOROFORM-d) δ=−120.78.

### Step 4: Synthesis of I-398

To a solution of Intermediate 1-6 (25 mg, 22.91 μmol, 1 eq) in DCM (0.25 mL) was added HCl/dioxane (2 M, 0.25 mL). The mixture was stirred at 25° C. for 0.5 hr. The reaction mixture was concentrated under reduced pressure to give a residue. The residue was dissolved in water (2 mL) and lyophilized to give I-398 (22.59 mg, 20.87 μmol, 91.08% yield, 94.917% purity, HCl salt) as a white solid. LCMS: Retention time: 0.334 min, (M+H)=991.6. ¹H NMR (400 MHz, METHANOL-d4) δ=¹H NMR (400 MHz, METHANOL-d4) δ=9.32 (s, 1H), 8.90 (d, J=5.2 Hz, 1H), 8.76 (s, 1H), 8.51 (s, 1H), 8.36 (d, J=7.6 Hz, 1H), 8.00-7.93 (m, 1H), 7.92-7.78 (m, 2H), 7.70 (d, J=5.6 Hz, 1H), 7.54-7.42 (m, 2H), 7.42-7.34 (m, 2H), 7.31 (d, J=7.6 Hz, 1H), 7.24-7.13 (m, 2H), 4.67 (s, 2H), 4.59 (d, J=17.6 Hz, 2H), 4.39 (s, 2H), 4.31 (s, 2H), 3.93 (s, 4H), 3.88-3.85 (m, 3H), 3.78 (d, J=4.4 Hz, 3H), 3.73 (s, 1H), 3.62-3.54 (m, 2H), 3.42 (s, 2H), 3.35 (d, J=6.4 Hz, 3H), 3.24 (s, 1H), 3.05-2.99 (m, 1H), 2.40-2.25 (m, 1H), 2.10 (d, J=12.4 Hz, 1H), 1.93 (d, J=11.6 Hz, 1H), 1.70-1.44 (m, 3H), 1.39-1.25 (m, 2H), 0.87-0.80 (m, 2H), 0.76-0.70 (m, 2H). ¹⁹F NMR (376 MHz, METHANOL-d4) δ=−120.70.

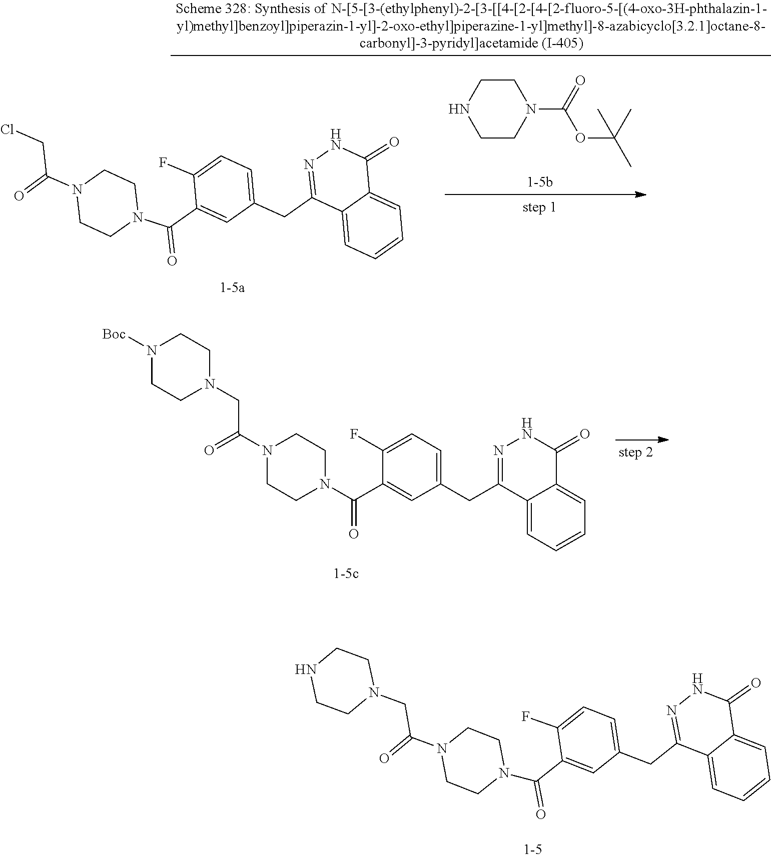 [SMILES: O=C(CCl)N1CCN(C(=O)c2cc(Cc3n[nH]c(=O)c4ccccc34)ccc2F)CC1 ; CC(C)(C)OC(=O)N1CCNCC1 ; CC(C)(C)OC(=O)N1CCN(CC(=O)N2CCN(C(=O)c3cc(Cc4n[nH]c(=O)c5ccccc45)ccc3F)CC2)CC1 ; O=C(CN1CCNCC1)N1CCN(C(=O)c2cc(Cc3n[nH]c(=O)c4ccccc34)ccc2F)CC1]

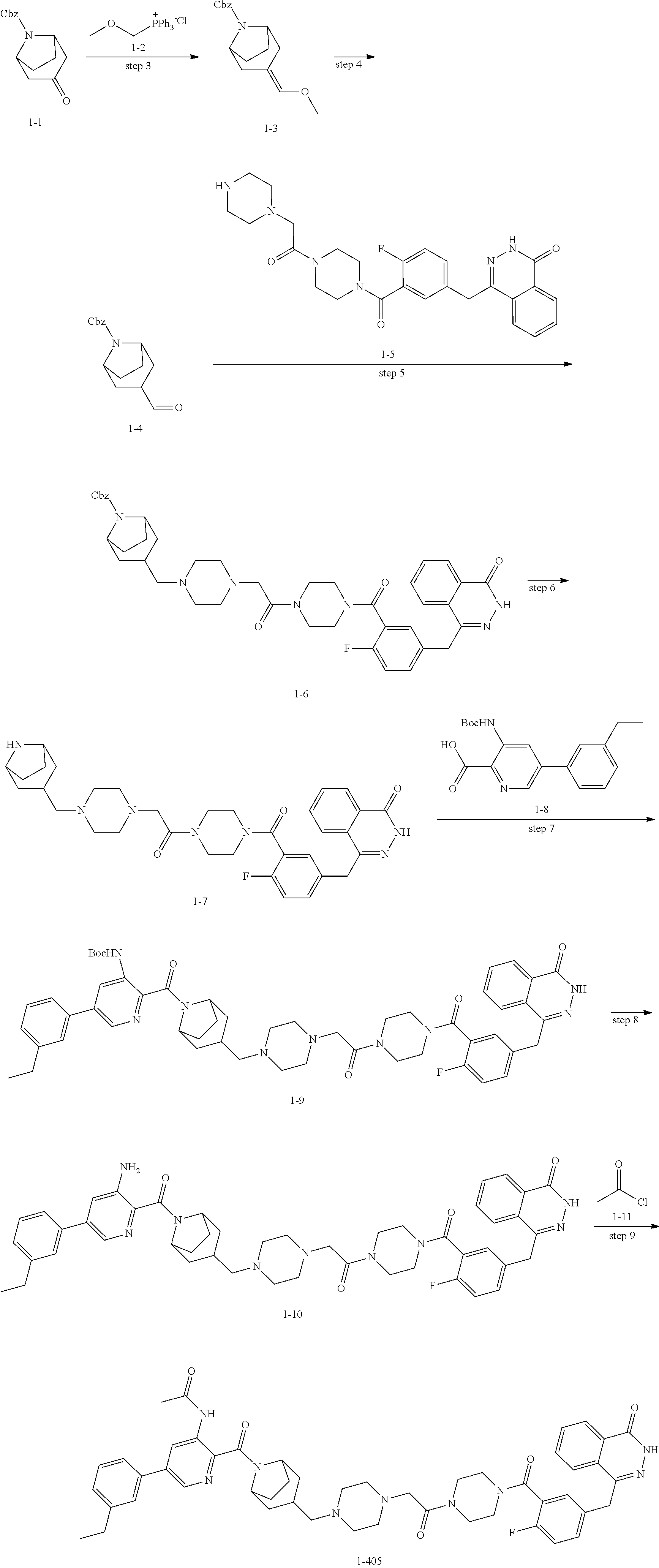

### Step 1: Synthesis of Intermediate 1-5c

To a mixture of Intermediate 1-5a (2 g, 4.52 mmol, 1 eq) in ACN (20 mL) was added DIEA (583.66 mg, 4.52 mmol, 786.60 μL, 1 eq) and Intermediate 1-5b (841.11 mg, 4.52 mmol, 1 eq). The mixture was stirred at 60° C. for 1 hr. The precipitate was filtered off, and ACN was removed by rotary evaporation. The residue was purified by reversed-phase HPLC (0.10% of FA) and the eluent was lyophilized to give product. Intermediate 1-5c (1.45 g, 2.33 mmol, 51.69% yield, 95.405% purity) was obtained as a white solid. LCMS (Method D)Retention time: 0.308 min, (M+H)=593.3.

### Step 2: Synthesis of Intermediate 1-5

A mixture of Intermediate 1-5c (0.9 g, 1.52 mmol, 1 eq) in HCl/dioxane (2 M, 9 mL) was stirred at 25° C. for 1 hr. The reaction mixture was concentrated under reduced pressure to give a residue. The crude product was used in the next step without further purification. Intermediate 1-5 (900 mg, crude, HCl salt) was obtained as a white solid. LCMS (Method D): Retention time: 0.245 min, (M+H)=493.1.

### Step 3: Synthesis of Intermediate 1-3

To a solution of Intermediate 1-2 (5.29 g, 15.43 mmol, 2 eq) in THF (10 mL) was added tBuOK (865.50 mg, 7.71 mmol, 1 eq) at −15° C., and the resultant mixture was stirred at the same temperature for 0.5 h. A solution of Intermediate 1-1 (2 g, 7.71 mmol, 1 eq) in THF (10 mL) was added dropwise to the reaction mixture at 25° C., and the mixture was then stirred for 10 h. The mixture was washed with water (20 mL) and extracted with DCM (20 mL*3), the combined organic phase was dried by Na₂SO₄, and concentrated under reduced pressure to give a residue. The residue was purified by reversed-phase HPLC (0.1% of FA) and the eluent was lyophilized to give a product. Intermediate 1-3 (650 mg, 1.89 mmol, 24.51% yield, 83.559% purity) was obtained as a brown oil. LCMS (Method D): Retention time: 0.464 min, (M+H)=288.1.

### Step 4: Synthesis of Intermediate 1-4

To a mixture of Intermediate 1-3 (200 mg, 696.01 μmol, 1 eq) in ACN (2 mL) was added NaI (31.30 mg, 208.80 μmol, 0.3 eq), CeCl₃ (68.62 mg, 278.40 μmol, 17.50 μL, 0.4 eq). The mixture was stirred at 80° C. for 10 hr. The mixture was washed with water (2 mL) and extracted with DCM (2 mL*3), the combined organic phase was dried by Na₂SO₄, and concentrated under reduced pressure to give a residue. The residue was purified by reversed-phase HPLC (0.1% of FA) and the eluent was lyophilized to give product. Intermediate 1-4 (405 mg, 1.08 mmol, 51.51% yield, 72.583% purity) was obtained as a yellow oil. LCMS (Method D): Retention time: 0.342 min, (M+H)=274.1.

### Step 5: Synthesis of Intermediate 1-6

To a solution of Intermediate 1-5 (629.02 mg, 1.19 mmol, 1 eq, HCl salt) in MeOH (3.5 mL) was added TEA (481.28 mg, 4.76 mmol, 662.00 μL, 4 eq) at 25° C. and the mixture was stirred at 25° C. for 10 min. Then Intermediate 1-4 (325 mg, 1.19 mmol, 1 eq), CH₃COOH (428.43 mg, 7.13 mmol, 408.42 μL, 6 eq) and NaBH₃CN (224.17 mg, 3.57 mmol, 3 eq) were added at 25° C. The resulting mixture was stirred at 25° C. for 10 hr. The mixture was diluted with water (4 mL) and extracted with EA (4 mL*3), the combined organic phase was dried by Na₂SO₄, and concentrated under reduced pressure to give a residue. The residue was purified by reversed-phase HPLC (0.1% of FA) and the eluent was lyophilized to give product. Intermediate 1-6 (383 mg, 482.46 μmol, 40.57% yield, 94.460% purity) was obtained as a white solid. LMCS (Method D): Retention time: 0.346 min, (M+H)=750.4

### Step 6: Synthesis of Intermediate 1-7

To a solution of Intermediate 1-6 (383 mg, 510.75 μmol, 1 eq) in ACN (4 mL) was added TMSI (306.60 mg, 1.53 mmol, 208.57 μL, 3 eq) and the reaction mixture was stirred at 25° C. for 1 hr. The reaction mixture was filtered. The reaction mixture was purified by reversed-phase HPLC (0.1% of FA) and the eluent was lyophilized to give product. Intermediate 1-7 (340 mg, 474.29 μmol, 92.86% yield, 85.893% purity) was obtained as a yellow solid. LCMS (Method D): Retention time: 0.231 min, (M+H)=616.3.

### Step 7: Synthesis of Intermediate 1-9

To a solution of Intermediate 1-7 (240 mg, 389.78 μmol, 1 eq) and Intermediate 1-8 (133.45 mg, 389.78 μmol, 1 eq) in DMF (2 mL) was added NMM (197.12 mg, 1.95 mmol, 214.26 μL, 5 eq), HOAT (106.11 mg, 779.55 μmol, 109.05 μL, 2 eq) and EDCI (373.60 mg, 1.95 mmol, 5 eq). The mixture was stirred at 25° C. for 1 hr. The mixture was diluted with water (2 mL) and extracted with DCM (2 mL*3), the combined organic phase was dried by Na₂SO₄, and concentrated under reduced pressure to give a residue. The residue was purified by flash silica gel chromatography (ISCO; 4 g SepaFlash Silica Flash Column, Eluent of 0˜50% Methanol/Dichloromethane @20 mL/min) and the eluent was concentrated to give product. Intermediate 1-9 (225 mg, 227.08 μmol, 58.26% yield, 94.882% purity) was obtained as a yellow solid. LCMS (Method D): Retention time: 0.484 min, (M+H)=940.4.

### Step 8: Synthesis of Intermediate 1-10

A solution of Intermediate 1-9 (225 mg, 239.33 μmol, 1 eq) in HCl/dioxane (2 M, 2.3 mL) was stirred at 25° C. for 1 hr. The reaction mixture was concentrated under reduced pressure to give a residue. The crude product was used in the next step without further purification. Intermediate 1-10 (225 mg, crude, HCl salt) was obtained as a yellow solid. LCMS (Method D): Retention time: 0.399 min, (M+H)=840.4.

### Step 9: Synthesis of Intermediate I-405

To a solution of Intermediate 1-10 (100 mg, 114.10 μmol, 1 eq, HCl salt) in DCM (1 mL) was added DIEA (29.49 mg, 228.19 μmol, 39.75 μL, 2 eq) and Intermediate 1-11 (10.75 mg, 136.91 μmol, 9.74 μL, 1.2 eq) and the mixture was stirred at 25° C. for 0.5 hr. The reaction mixture was filtered and concentrated under reduced pressure to give a residue. The residue was purified by reversed-phase HPLC (0.1% of FA) and the eluent was lyophilized to give product. I-405 (18 mg, 36.93 μmol, 32.37% yield, 98.712% purity) was obtained as a white solid. LCMS (Method D): Retention time: 0.399 min, (M+H)=882.4. ¹H NMR (400 MHz, METHANOL-d4) δ=8.63 (d, J=1.6 Hz, 1H), 8.50-8.45 (m, 1H), 8.37 (d, J=7.6 Hz, 1H), 7.98-7.91 (m, 1H), 7.91-7.78 (m, 2H), 7.55-7.46 (m, 3H), 7.42 (t, J=7.6 Hz, 1H), 7.40-7.34 (m, 1H), 7.31 (d, J=7.6 Hz, 1H), 7.16 (t, J=8.8 Hz, 1H), 4.38 (s, 3H), 3.82-3.72 (m, 2H), 3.68 (s, 2H), 3.52 (s, 2H), 3.38 (s, 5H), 2.88-2.53 (m, 10H), 2.46-2.27 (m, 3H), 2.18 (s, 3H), 2.11-2.00 (m, 2H), 1.91-1.78 (m, 3H), 1.76-1.65 (m, 2H), 1.61-1.50 (m, 1H), 1.29 (t, J=7.6 Hz, 3H).

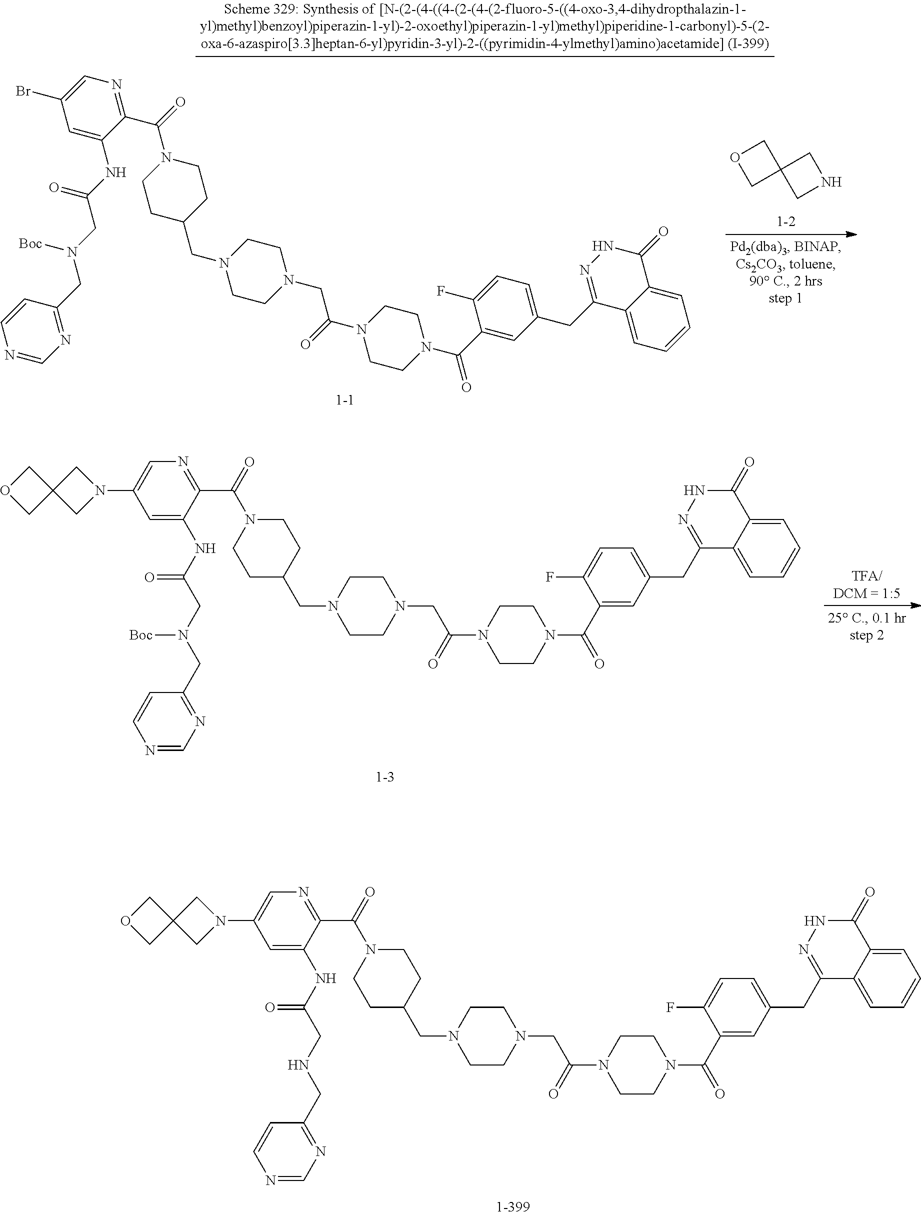 [SMILES: CC(C)(C)OC(=O)N(CC(=O)Nc1cc(Br)cnc1C(=O)N1CCC(CN2CCN(CC(=O)N3CCN(C(=O)c4cc(Cc5n[nH]c(=O)c6ccccc56)ccc4F)CC3)CC2)CC1)Cc1ccncn1 ; C1NCC12COC2 ; CC(C)(C)OC(=O)N(CC(=O)Nc1cc(N2CC3(COC3)C2)cnc1C(=O)N1CCC(CN2CCN(CC(=O)N3CCN(C(=O)c4cc(Cc5n[nH]c(=O)c6ccccc56)ccc4F)CC3)CC2)CC1)Cc1ccncn1 ; O=C(CNCc1ccncn1)Nc1cc(N2CC3(COC3)C2)cnc1C(=O)N1CCC(CN2CCN(CC(=O)N3CCN(C(=O)c4cc(Cc5n[nH]c(=O)c6ccccc56)ccc4F)CC3)CC2)CC1]

### Step 1: Synthesis of Intermediate 1-3.

A mixture of Intermediate 1-1 (120 mg, 115.61 μmol, 1 eq), Intermediate 1-2 (12.61 mg, 127.17 mol, 1.1 eq), Cs₂CO₃ (37.67 mg, 115.61 μmol, 1 eq), BINAP (3.60 mg, 5.78 μmol, 0.05 eq) and Pd₂(dba)₃ (2.12 mg, 2.31 μmol, 0.02 eq) in toluene (1 mL) was degassed and purged with N₂ 3 times, and then the mixture was stirred at 90° C. for 2 hr under N₂ atmosphere. The reaction mixture was diluted with H₂O (5 mL) and extracted with EA (5 mL*3). The combined organic layers were washed with brine (5 mL*3), dried over Na₂SO₄, filtered and concentrated under reduced pressure to give a residue. The residue was purified by prep-HPLC (neutral condition), the eluent was concentrated and lyophilized to give the desired product. Intermediate 1-3 (30 mg, 22.11 μmol, 19.12% yield, 77.841% purity) was obtained as a brown oil. LCMS (Method D): Retention time: 0.324 min, (M/2+H)=528.8.

### Step 2: Synthesis of I-399

To a solution of Intermediate 1-3 (25 mg, 23.67 μmol, 1 eq) in DCM (0.5 mL) was added TFA (0.1 mL). The mixture was stirred at 25° C. for 0.1 hr. The reaction mixture was concentrated under reduced pressure to give a residue. The residue was purified by prep-HPLC (TFA condition), the eluent was concentrated and lyophilized to give a product. I-399 (7.21 mg, 6.48 μmol, 27.39% yield, 96.221% purity, TFA salt) was obtained as a yellow solid. LCMS (Method F): Retention time: 0.486 min, (M+H)=956.5. ¹H NMR (400 MHz, METHANOL-d4) δ=9.25 (s, 1H), 8.84 (d, J=5.2 Hz, 1H), 8.37 (d, J=7.6 Hz, 1H), 8.01-7.93 (m, 1H), 7.92-7.80 (m, 2H), 7.66-7.57 (m, 2H), 7.52 (d, J=4.8 Hz, 1H), 7.40-7.31 (m, 1H), 7.25 (s, 1H), 7.21-7.12 (m, 1H), 4.68-4.52 (m, 4H), 4.39 (s, 2H), 4.21-4.12 (m, 6H), 3.96-3.44 (m, 12H), 3.24-3.11 (m, 5H), 3.09-2.97 (m, 4H), 2.94-2.76 (m, 4H), 2.19-2.07 (m, 1H), 1.97-1.87 (m, 1H), 1.79-1.66 (m, 1H), 1.57-1.23 (m, 3H). ¹⁹F NMR (376 MHz, METHANOL-d4) δ=−77.19, −120.79.

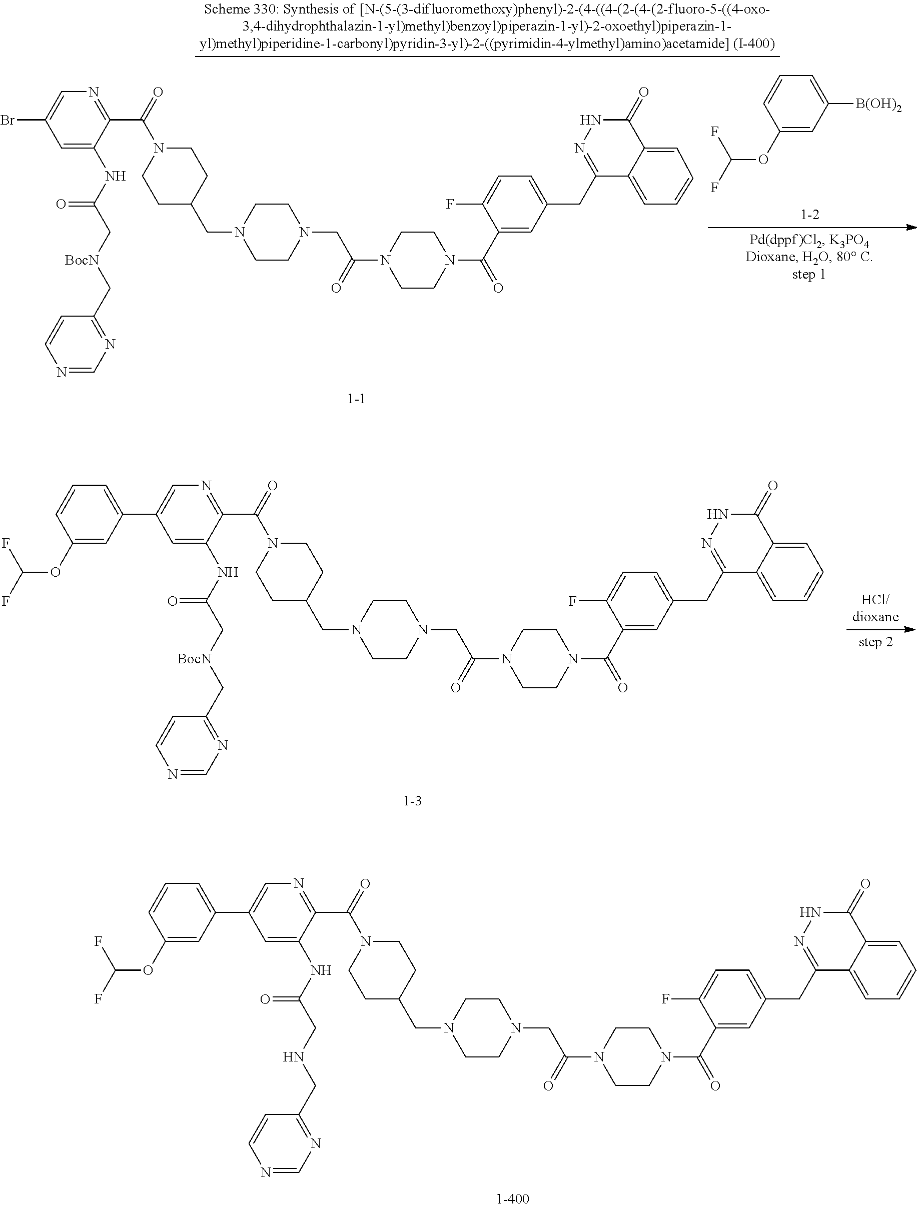

### Step 1: Synthesis of Intermediate 1-3

A mixture of Intermediate 1-1 (50 mg, 48.17 μmol, 1 eq), Intermediate 1-2 (9.05 mg, 48.17 μmol, 1 eq), ditert-butyl(cyclopentyl)phosphane;dichloropalladium;iron (3.14 mg, 4.82 μmol, 0.1 eq), and K₃PO₄ (30.68 mg, 144.51 μmol, 3 eq) in dioxane (0.5 mL) and H₂O (0.1 mL) was degassed and purged with N₂ 3 times, and then the mixture was stirred at 80° C. for 1 hr under N₂ atmosphere. The reaction mixture was filtered and concentrated under reduced pressure to give a residue. The residue was purified by prep-HPLC (column: Phenomenex luna C18 150*25 mm*10 um; mobile phase: [water(FA)-ACN]; gradient: 18%-48% B over 10 min) and the eluent was concentrated to remove ACN and lyophilized to give Intermediate 1-3 (40 mg, 36.32 μmol, 75.41% yield) as a white solid. LCMS (Method D): Retention time: 0.423 min, (M+H)=1101.5.

### Step 2: Synthesis of I-400

A solution of Intermediate 1-3 (40 mg, 36.32 μmol, 1 eq) in HCl/dioxane (0.5 mL) was stirred at 25° C. for 0.5 hr. The reaction mixture was concentrated under reduced pressure to give a residue. The residue was concentrated to remove ACN and lyophilized to give I-400 (26.1 mg, 23.93 μmol, 65.87% yield, 95.108% purity, HCl salt) as a white solid. LCMS (Method D): Retention time: 0.343 min, (M+H)=1001.5. ¹H NMR (400 MHz, METHANOL-d4) δ=9.27 (s, 1H), 8.86 (d, J=5.2 Hz, 1H), 8.72 (d, J=1.6 Hz, 1H), 8.42-8.35 (m, 2H), 7.99-7.93 (m, 1H), 7.92-7.82 (m, 2H), 7.62 (d, J=5.2 Hz, 1H), 7.60-7.56 (m, 2H), 7.54-7.50 (m, 1H), 7.48 (s, 1H), 7.41-7.34 (m, 1H), 7.31-7.25 (m, 1H), 7.20-7.15 (m, 1H), 7.13-6.76 (m, 1H), 4.67 (d, J=2.4 Hz, 1H), 4.61 (s, 2H), 4.39 (s, 2H), 4.34-4.31 (m, 2H), 4.26 (s, 2H), 3.88-3.77 (m, 4H), 3.74-3.64 (m, 6H), 3.61-3.54 (m, 4H), 3.41 (s, 2H), 3.23 (d, J=12.8 Hz, 3H), 3.02-2.94 (m, 1H), 2.32-2.22 (m, 1H), 2.05 (d, J=12.6 Hz, 1H), 1.91-1.84 (m, 1H), 1.61 (d, J=10.8 Hz, 1H), 1.52-1.44 (m, 1H), 1.37-1.30 (m, 1H).

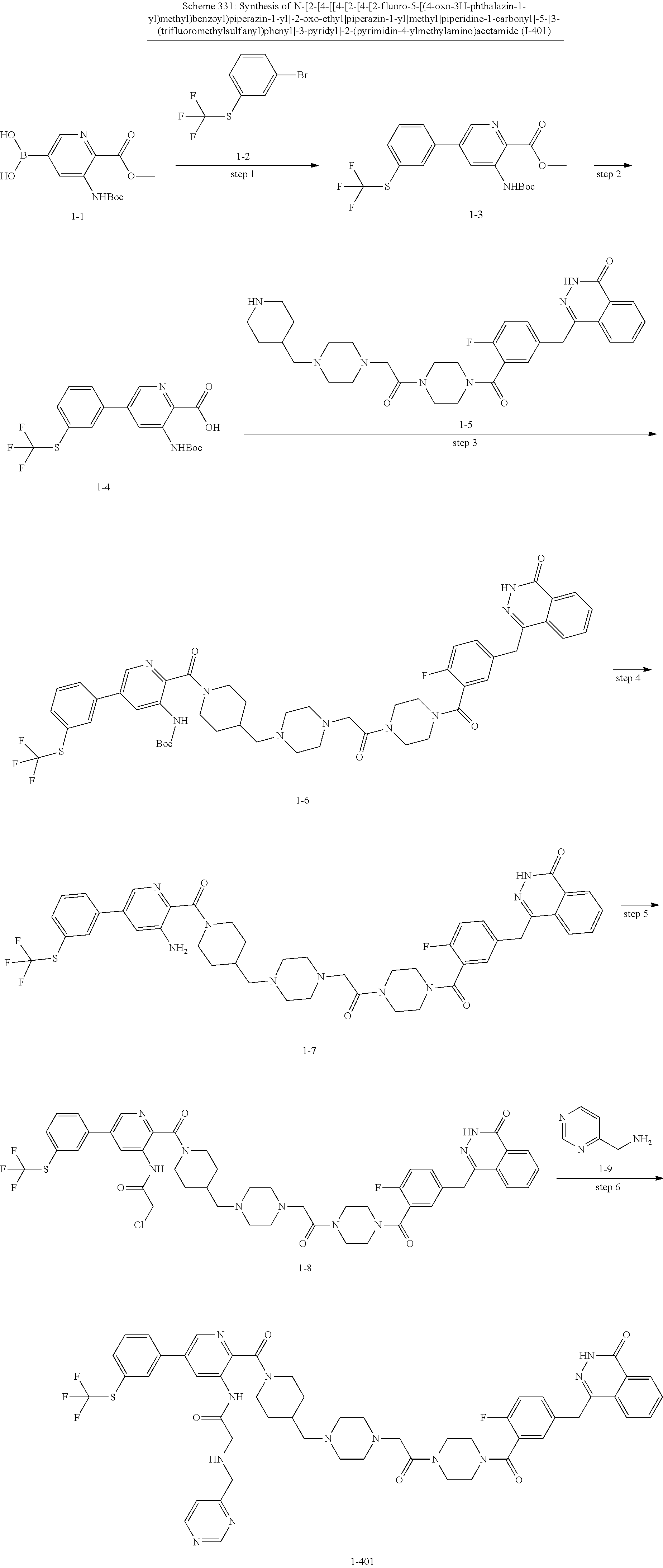

### Step 1: Synthesis of Intermediate 1-3

A mixture of Intermediate 1-1 (276.42 mg, 933.60 μmol, 1.2 eq), Intermediate 1-2 200 mg, 778.00 μmol, 1 eq), ditert-butyl(cyclopentyl)phosphane;dichloropalladium;iron (50.71 mg, 77.80 μmol, 0.1 eq) and K₃PO₄ (495.43 mg, 2.33 mmol, 3 eq) in dioxane (2 mL) and H₂O (0.4 mL) was degassed and purged with N₂ 3 times, and then the mixture was stirred at 80° C. for 1 hr under N₂ atmosphere. The reaction mixture was diluted with water (10 mL) and extracted with EA (5 mL*3). The combined organic layers were dried over Na₂SO₄, filtered and concentrated under reduced pressure to give a residue. The residue was purified by flash silica gel chromatography (ISCO; 20 g SepaFlash Silica Flash Column, Eluent of 0˜100% Petroleum ether/Ethyl acetate @70 mL/min). the eluent was concentrated under reduced pressure to give a residue. Intermediate 1-3 (245 mg, 571.86 μmol, 73.50% yield) was obtained as a yellow solid. LCMS (Method D): Rt=0.505 min, M+H=429.1.

### Step 2: Synthesis of Intermediate 1-4

To a solution of Intermediate 1-3 (240 mg, 560.19 μmol, 1 eq) in THF (0.8 mL), MeOH (0.8 mL), and H₂O (0.8 mL) was added LiOH·H₂O (70.52 mg, 1.68 mmol, 3 eq). The mixture was stirred at 25° C. for 1 hr. The mixture was adjusted to pH=5 with CA and extracted with EA 15 mL (5 mL*3). The combined organic layers were dried over Na₂SO₄, filtered and concentrated under reduced pressure to give a residue. The residue was used in the next step without further purification. Intermediate 1-4 (210 mg, 506.76 μmol, 90.46% yield) was obtained as a yellow solid. LCMS (Method D): Rt=0.508 min, M+H=415.1.

### Step 3: Synthesis of Intermediate 1-6

To a solution of Intermediate 1-4 (180 mg, 434.36 μmol, 1 eq) in DMF (2 mL) was added HOAt (59.12 mg, 434.36 μmol, 60.76 μL, 1 eq) and EDCI (249.80 mg, 1.30 mmol, 3 eq), NMM (219.68 mg, 2.17 mmol, 238.78 μL, 5 eq), Intermediate 1-5 (271.98 mg, 434.36 μmol, 1 eq, HCl salt). The mixture was stirred at 25° C. for 1 hr. The reaction mixture was diluted with water (10 mL) and extracted with EA (5 mL*3). The combined organic layers were dried over Na₂SO₄, filtered and concentrated under reduced pressure to give a residue. The residue was purified by prep-HPLC (0.1% FA condition) and the eluent was lyophilized to give product. Intermediate 1-6 (130 mg, 131.83 μmol, 30.35% yield) was obtained as a yellow oil. LCMS (Method D): Rt=0.413 min, M+H=986.4. ¹H NMR (400 MHz, METHANOL-d4) δ=8.56 (d, J=2.0 Hz, 1H), 8.44 (d, J=2.0 Hz, 1H), 8.37 (d, J=7.2 Hz, 1H), 8.02 (s, 1H), 7.98-7.78 (m, 5H), 7.69-7.62 (m, 1H), 7.55-7.46 (m, 1H), 7.39-7.34 (m, 1H), 7.17 (t, J=9.2 Hz, 1H), 4.68 (d, J=12.8 Hz, 1H), 4.39 (s, 2H), 3.93-3.75 (m, 3H), 3.73-3.58 (m, 4H), 3.55-3.45 (m, 2H), 3.23-3.07 (m, 5H), 3.03-2.89 (m, 5H), 2.86-2.78 (m, 2H), 2.14-2.06 (m, 1H), 2.00-1.90 (m, 1H), 1.80-1.73 (m, 1H), 1.53 (s, 9H), 1.50-1.43 (m, 1H), 1.41-1.26 (m, 3H).

### Step 4: Synthesis of Intermediate 1-7

To a solution of Intermediate 1-6 (110 mg, 111.55 μmol, 1 eq) in DCM (0.5 mL) was added HCl/dioxane (2 M, 0.5 mL, 8.96 eq). The mixture was stirred at 25° C. for 1 hr. The reaction mixture was concentrated under reduced pressure to give a residue. The residue was used in the next step without further purification. Intermediate 1-7 (95 mg, 107.23 μmol, 96.12% yield) was obtained as a yellow oil. LCMS (Method D): Rt=0.378 min, M+H=886.3.

### Step 5: Synthesis of Intermediate 1-8

To a solution of Intermediate 1-7 (60 mg, 65.05 μmol, 1 eq, HCl salt) in DCM (1 mL) was added TEA (19.75 mg, 195.14 μmol, 27.16 μL, 3 eq), then 2-chloroacetyl chloride (7.35 mg, 65.05 μmol, 5.18 L, 1 eq) was added at 0° C. The mixture was stirred at 0° C. for 1 hr. The reaction mixture was concentrated under reduced pressure to give a residue. The residue was used in the next step without further purification. Intermediate 1-8 (50 mg, 51.95 μmol, 79.87% yield) was obtained as a brown oil. LCMS (Method D): Rt=0.393 min, M+H=962.4.

### Step 6: Synthesis of I-401

To a solution of Intermediate 1-8 (50 mg, 51.95 μmol, 1 eq) and Intermediate 1-9 (8.50 mg, 77.93 μmol, 1.5 eq) in ACN (1 mL) was added DIEA (26.86 mg, 207.80 μmol, 36.20 μL, 4 eq) and KI (8.62 mg, 51.95 μmol, 1 eq). The mixture was stirred at 40° C. for 1.5 hr. The reaction mixture was concentrated under reduced pressure to give a residue. The residue was purified by prep-HPLC (column: Phenomenex luna C18 150*25 mm*10 um; mobile phase: [water(FA)-ACN]; gradient: 13%-43% B over 10 min), and the eluent was lyophilized to give product. I-401 (8 mg, 7.19 μmol, 13.83% yield, 97.119% purity, FA salt) was obtained as a yellow solid. LCMS (Method D): Rt=0.338 min, M+H=1035.3. ¹H NMR (400 MHz, METHANOL-d4) δ=9.10 (s, 1H), 8.83-8.73 (m, 2H), 8.64 (d, J=2.0 Hz, 1H), 8.37 (d, J=7.6 Hz, 1H), 8.31 (s, 1H), 8.04 (s, 1H), 7.98-7.90 (m, 2H), 7.90-7.82 (m, 2H), 7.82 (s, 2H), 7.70-7.65 (m, 1H), 7.50 (d, J=3.2 Hz, 1H), 7.39-7.34 (m, 1H), 7.17 (t, J=8.8 Hz, 1H), 4.65-4.58 (m, 1H), 4.39 (s, 2H), 4.02 (s, 2H), 3.90-3.62 (m, 6H), 3.55-3.49 (m, 5H), 3.43 (s, 1H), 3.16 (t, J=12.0 Hz, 1H), 2.91-2.71 (m, 9H), 2.60-2.50 (m, 2H), 2.05-1.95 (m, 1H), 1.93-1.85 (m, 1H), 1.79-1.72 (m, 1H), 1.39-1.28 (m, 2H), 1.22 (d, J=12.0 Hz, 1H).

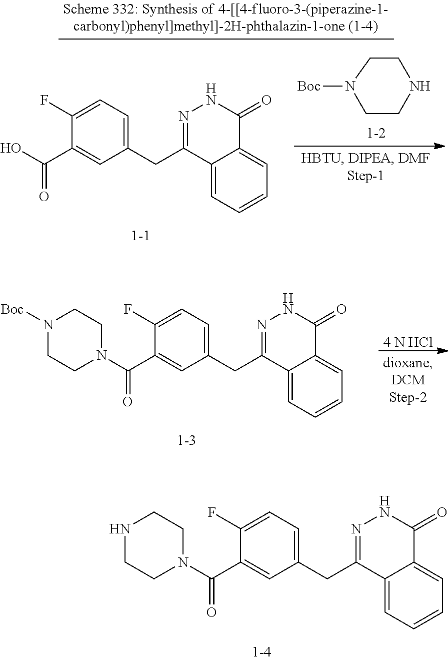

### Step 1: Synthesis of Intermediate 1-3

A solution of Intermediate 1-1 (300 g, 1.01 μmol, 1 eq), Intermediate 1-2 (206.07 g, 1.11 μmol, 1.1 eq), HBTU (457.73 g, 1.21 μmol, 1.2 eq) and TEA (203.55 g, 2.01 μmol, 279.99 mL, 2 eq) in DMF (2000 mL) was stirred at 20-25° C. for 1.5 hrs under N₂. To the reaction mixture was added to water (2 L) dropwise while stirring, and kept at 0-5° C. (ice bath) for 1 h. Then the precipitate was filtered off and collected. The collected solid was washed with ice cold water 3 times, then dried under reduced pressure. The solid was recrystallized in acetonitrile (1 L, at 20-25° C.) to afford the target product. Intermediate 1-3 (433 g, 928.18 mmol, 92.28% yield) was obtained as a white solid. LCMS (Method D): Rt=0.499 min, [M+Na]⁺=489.2. ¹H NMR (400 MHz, DMSO-d6) δ=12.60 (s, 1H), 8.26 (d, J=7.8 Hz, 1H), 7.99-7.93 (m, 1H), 7.92-7.81 (m, 2H), 7.47-7.40 (m, 1H), 7.35 (dd, J=1.8, 6.4 Hz, 1H), 7.23 (t, J=9.0 Hz, 1H), 4.33 (s, 2H), 3.59 (br s, 2H), 3.38-3.36 (m, 1H), 3.33-3.32 (m, 1H), 3.23 (br s, 2H), 3.14 (br s, 2H), 2.89 (s, 1H), 2.73 (s, 1H), 2.56-2.53 (m, 1H), 1.40 (s, 9H).

### Step 2: Synthesis of 1-4

To a suspension of Intermediate 1-3 (215 g, 460.88 mmol, 1 eq) in MeOH (200 mL) was added HCl/MeOH (4 M, 1.00 μL, 8.68 eq) portion-wisely. The system was stirred for 16 hrs at 25° C. The reaction was concentrated directly to afford the crude. The crude was combined with another batch of same scale and the combined crude was purified together. The crude was suspended in THF (1 L) and basified by NH₃·H₂O. The suspension was stirred for 16 hrs until the crystal were fully formed. The crystals were filtered off and collected. 1-4 (519 g, 1.37 μmol, 74.53% yield, 97% purity) was obtained as white solid. LCMS (Method E): Rt=0.356 min, [M+H]⁺=367.1.

¹H NMR (400 MHz, DMSO-d6) δ=12.97-12.24 (m, 1H), 8.28 (d, J=7.6 Hz, 1H), 7.97 (d, J=7.3 Hz, 1H), 7.89 (t, J=7.0 Hz, 1H), 7.86-7.81 (m, 1H), 7.44-7.39 (m, 1H), 7.37-7.32 (m, 1H), 7.22 (t, J=9.0 Hz, 1H), 4.34 (s, 2H), 3.54-3.44 (m, 2H), 3.10 (s, 2H), 2.74 (t, J=4.6 Hz, 2H), 2.63-2.55 (m, 2H), 2.52 (s, 1H)

¹H NMR (400 MHz, DMSO-d6) δ=8.24 (d, J=7.7 Hz, 1H), 7.94-7.76 (m, 3H), 7.43-7.32 (m, 1H), 7.27 (d, J=4.9 Hz, 1H), 7.17 (t, J=9.0 Hz, 1H), 4.29 (s, 2H), 3.53 (s, 2H), 3.07 (s, 2H), 2.69 (t, J=4.4 Hz, 2H), 2.58-2.52 (m, 2H).

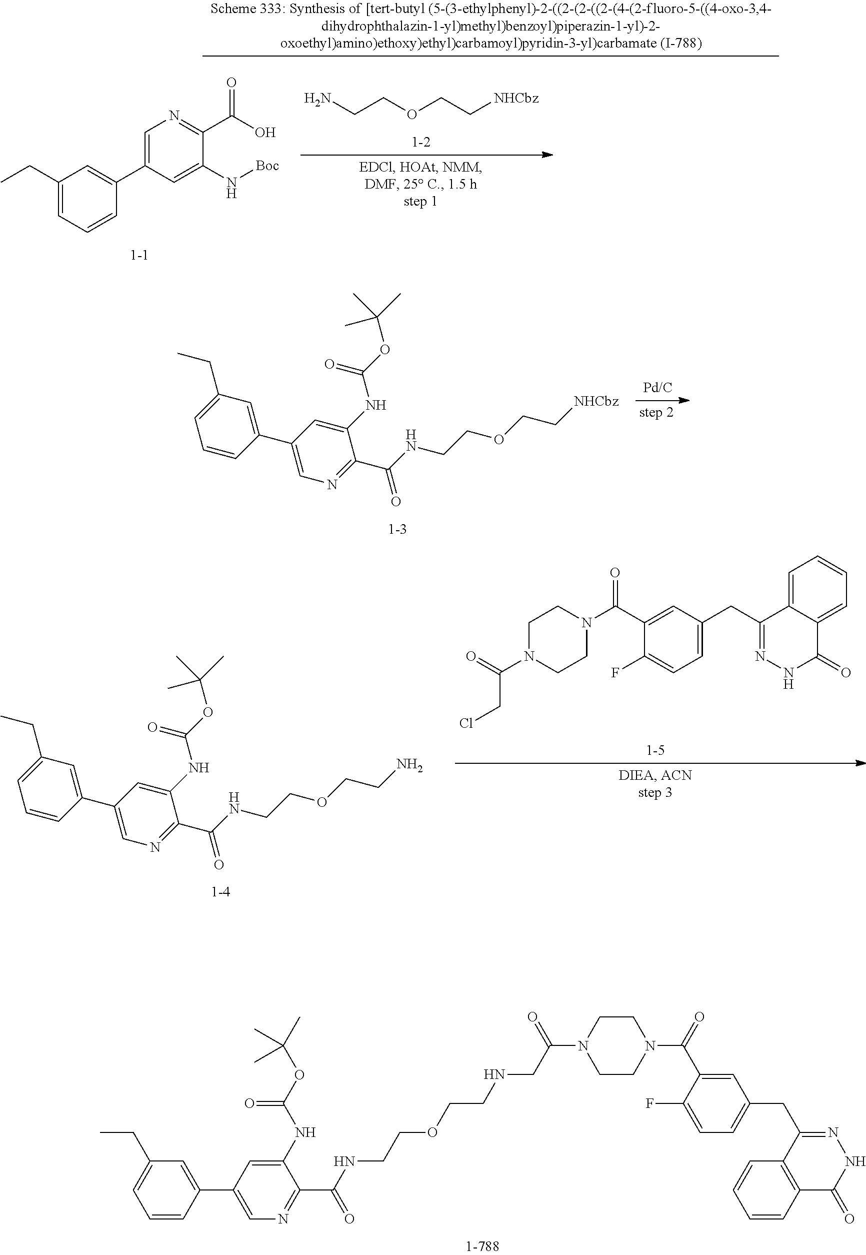 [SMILES: CCc1cccc(-c2cnc(C(=O)O)c(NC(=O)OC(C)(C)C)c2)c1 ; NCCOCCNC(=O)OCc1ccccc1 ; CCc1cccc(-c2cnc(C(=O)NCCOCCNC(=O)OCc3ccccc3)c(NC(=O)OC(C)(C)C)c2)c1 ; CCc1cccc(-c2cnc(C(=O)NCCOCCN)c(NC(=O)OC(C)(C)C)c2)c1 ; O=C(CCl)N1CCN(C(=O)c2cc(Cc3n[nH]c(=O)c4ccccc34)ccc2F)CC1 ; CCc1cccc(-c2cnc(C(=O)NCCOCCNCC(=O)N3CCN(C(=O)c4cc(Cc5n[nH]c(=O)c6ccccc56)ccc4F)CC3)c(NC(=O)OC(C)(C)C)c2)c1]

### Step 1: Synthesis of Intermediate 1-3

To a solution of Intermediate 1-1 (200 mg, 584.13 μmol, 1 eq) and Intermediate 1-2 (139.19 mg, 584.13 μmol, 1 eq) in DMF (2 mL) was added EDCI (335.94 mg, 1.75 mmol, 3 eq) and HOAt (79.51 mg, 584.13 μmol, 81.71 μL, 1 eq), NMM (295.42 mg, 2.92 mmol, 321.10 μL, 5 eq). The mixture was stirred at 25° C. for 1 hr. The reaction mixture was filtered and concentrated under reduced pressure to give a residue. The residue was purified by prep-HPLC (0.1% FA) the eluent was concentrated to remove ACN to give Intermediate 1-3 (0.2 g, 355.46 μmol, 60.85% yield) as colorless oil. LCMS (Method D): Retention time: 0.587 min, (M+H)=563.5.

### Step 2: Synthesis of Intermediate 1-4

A mixture of Intermediate 1-3 (200 mg, 355.46 μmol, 1 eq), Pd/C (100 mg, 93.97 μmol, 10% purity, 2.64e−1 eq) in MeOH (2 mL) was degassed and purged with H₂ 3 times, and then the mixture was stirred at 25° C. for 1 hr under H₂ atmosphere. The reaction mixture was filtered and concentrated under reduced pressure to give Intermediate 1-4 (0.1 g, 233.36 μmol, 65.65% yield) as colorless oil. LCMS (Method D): Retention time: 0.417 min, (M+H)=701.4.

### Step 3: Synthesis of I-788

To a solution of Intermediate 1-4 (0.1 g, 233.36 μmol, 1 eq) and 4-[[3-[4-(2-chloroacetyl)piperazine-1-carbonyl]-4-fluoro-phenyl]methyl]-2H-phthalazin-1-one (124.02 mg, 280.03 μmol, 1.2 eq) in ACN (1 mL) was added DIEA (90.48 mg, 700.08 μmol, 121.94 μL, 3 eq). The mixture was stirred at 40° C. for 0.5 hr. The reaction mixture was filtered and concentrated under reduced pressure to give a residue. The residue was purified by prep-HPLC (column: Phenomenex luna C18 150*25 mm*10 um; mobile phase: [water(FA)-ACN]; gradient: 30%-60% B over 10 min) and the eluent was concentrated to remove ACN and lyophilized to give I-788 (80 mg, 88.21 μmol, 37.80% yield, 97.137% purity, FA salt) was obtained as a white solid. LCMS (Method D): Retention time: 0.444 min, (M+H)=835.5. ¹H NMR (400 MHz, METHANOL-d4) δ=9.03-8.96 (m, 1H), 8.56 (s, 1H), 8.53-8.44 (m, 1H), 8.41-8.32 (m, 1H), 7.97-7.90 (m, 1H), 7.89-7.76 (m, 2H), 7.55-7.45 (m, 3H), 7.44-7.35 (m, 1H), 7.35-7.24 (m, 2H), 7.19-7.03 (m, 1H), 4.36 (d, J=6.0 Hz, 2H), 3.78-3.56 (m, 12H), 3.51-3.43 (m, 2H), 3.34 (d, J=2.0 Hz, 1H), 3.22 (d, J=4.0 Hz, 1H), 2.98-2.87 (m, 2H), 2.79-2.67 (m, 2H), 1.56-1.53 (m, 9H), 1.32-1.23 (m, 3H).

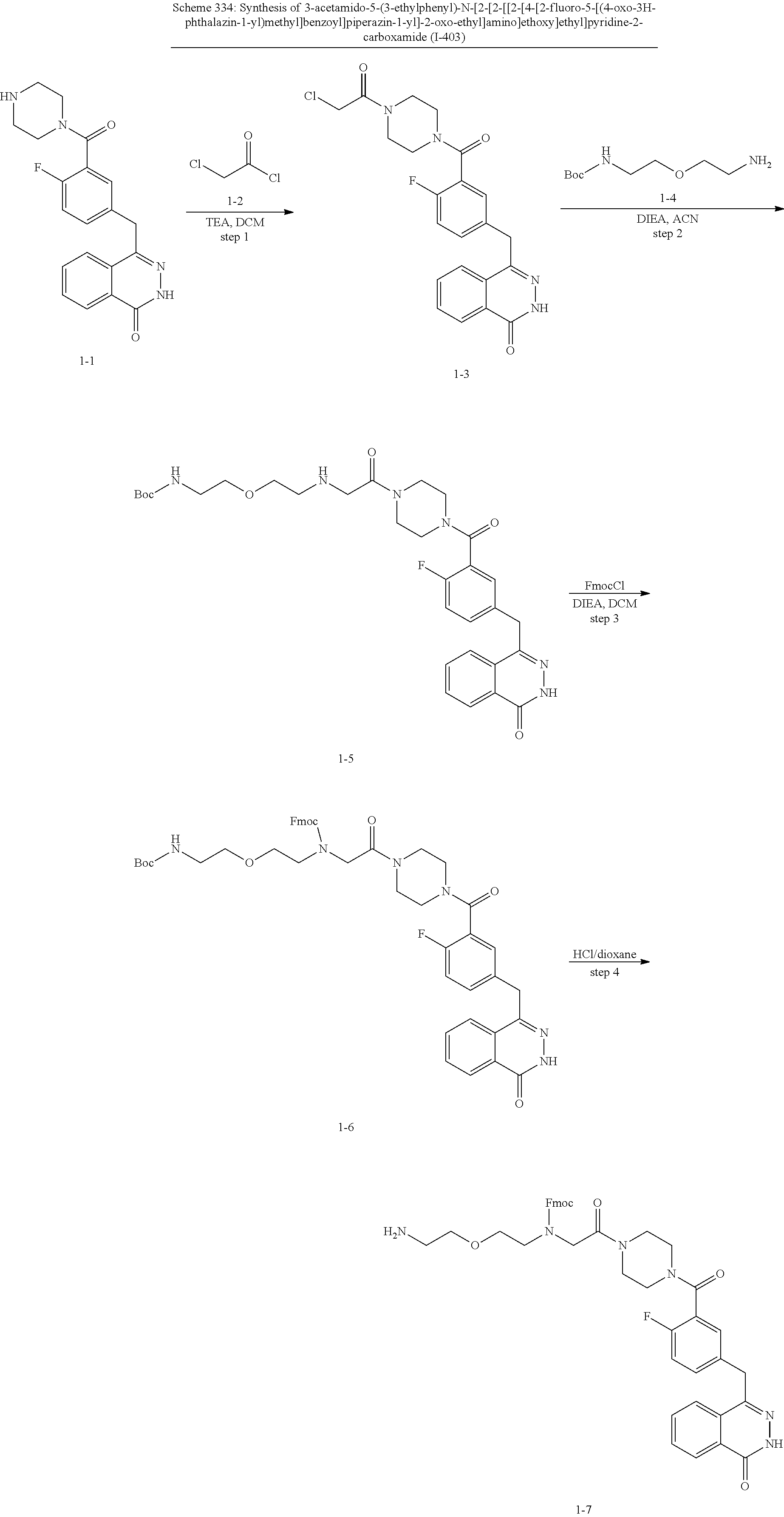

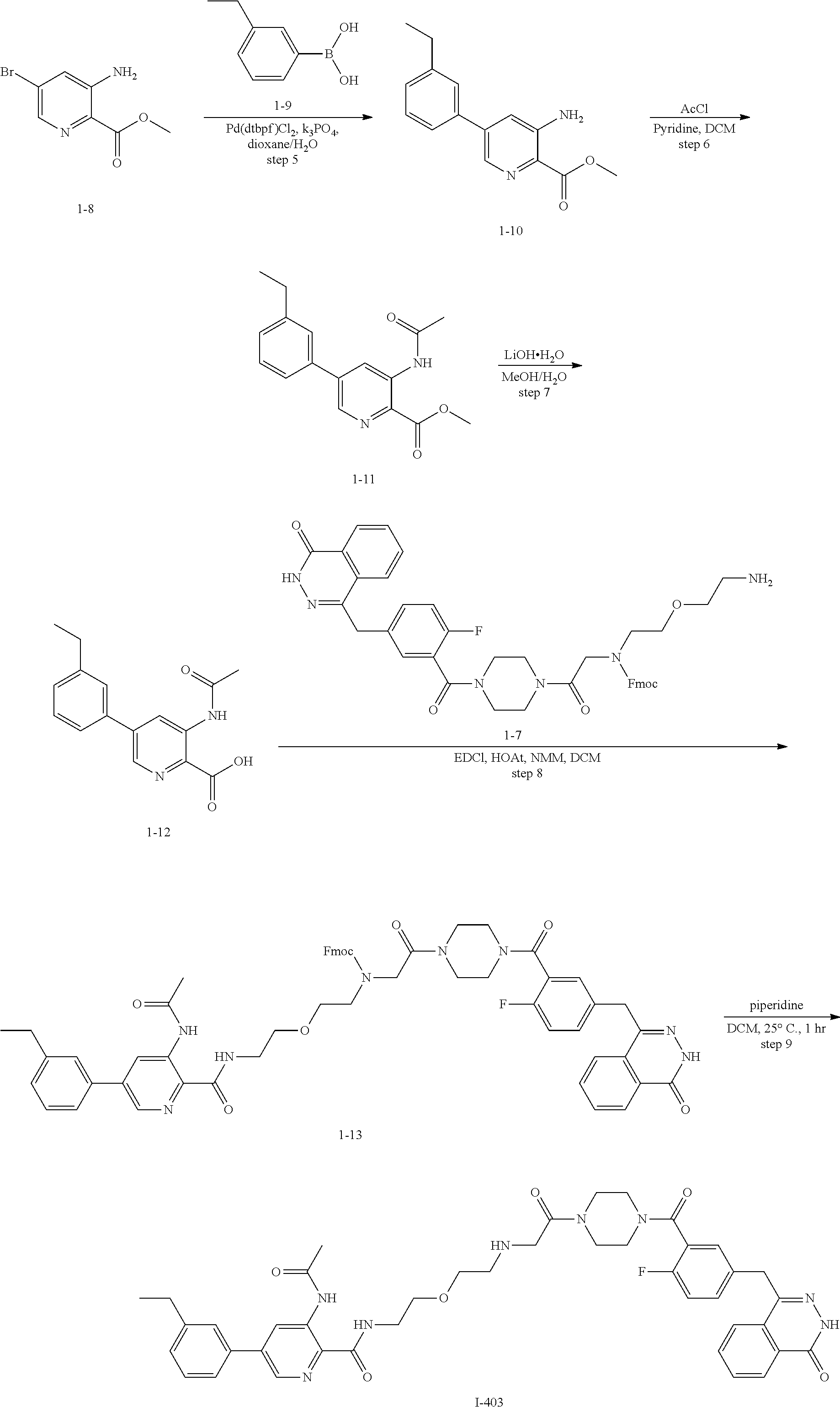

### Step 1: Synthesis of Intermediate 1-3

To a solution of Intermediate 1-1 (10 g, 27.29 mmol, 1 eq) and TEA (8.29 g, 81.88 mmol, 11.40 mL, 3 eq) in DCM (100 mL) was added Intermediate 1-2 (4.62 g, 40.94 mmol, 3.26 mL, 1.5 eq) at 0° C. Then the mixture was stirred at 0° C. for 1 hr. The reaction mixture was poured into water (200 mL) and extracted with DCM (200 mL*2), the organic layer was washed with brine (200 mL) and dried over Na₂SO₄. The reaction mixture was concentrated to give the crude product as a yellow solid. Intermediate 1-3 (12 g, 26.15 mmol, 95.80% yield, 96.5% purity) was obtained as a yellow solid. LCMS (Method E): Rt=0.428 min, [M+H]⁺=443.2.

### Step 2: Synthesis of Intermediate 1-5

To a solution of Intermediate 1-3 (11.5 g, 25.97 mmol, 1 eq) and DIEA (16.78 g, 129.83 mmol, 22.61 mL, 5 eq) in DCM (120 mL) was added Intermediate 1-4 (7.96 g, 38.95 mmol, 1.5 eq) at 40° C. Then the mixture was stirred at 40° C. for 16 hr. The reaction mixture was concentrated to give Intermediate 1-5 (16 g, 15.35 mmol, 59.13% yield, 58.6% purity) as yellow oil. The crude product was used for the next step without further purification. LCMS (Method G): Rt=0.503 min, [M+H]⁺=611.4.

### Step 3: Synthesis of Intermediate 1-6

To a solution of Intermediate 1-5 (16 g, 26.20 mmol, 1 eq) and DIEA (10.16 g, 78.60 mmol, 13.69 mL, 3 eq) in DCM (160 mL) was added 9H-fluoren-9-ylmethyl carbonochloridate (10.17 g, 39.30 mmol, 1.5 eq) at 0° C. Then the mixture was stirred at 25° C. for 1 hr. The reaction mixture was poured into water (30 mL) and extracted with DCM (30 mL*2), the organic layer was washed with brine (30 mL) and dried over Na₂SO₄. The combined organics were concentrated to give the crude product. The crude product was purified by flash silica gel column chromatography (SiO₂, PE:EA=10:1 to 1:1) to give Intermediate 1-6 (11 g, 12.61 mmol, 48.14% yield, 95.5% purity) as yellow solid. LCMS (Method E): Rt=0.561 min, [M+H]⁺=833.4. ¹H NMR (400 MHz, CHLOROFORM-d) δ=10.44 (br d, J=13.6 Hz, 1H), 8.63-8.33 (m, 1H), 7.85-7.68 (m, 5H), 7.64-7.53 (m, 2H), 7.45-7.29 (m, 6H), 7.15-7.01 (m, 1H), 5.10-4.82 (m, 1H), 4.67-4.46 (m, 2H), 4.38-4.20 (m, 3H), 3.98-3.61 (m, 4H), 3.60-3.42 (m, 5H), 3.40-3.20 (m, 8H), 1.47-1.41 (m, 9H).

### Step 4: Synthesis of Intermediate 1-7

A solution of Intermediate 1-6 (10 g, 12.01 mmol, 1 eq) in HCl/dioxane (100 mL) was stirred at 25° C. for 1 hr. The reaction mixture was concentrated to give the crude product. The crude product was triturated with EA (100 mL) at 25° C. for 30 min to give Intermediate 1-7 (9.5 g, 11.41 mmol, 95.04% yield, 92.4% purity, HCl salt) as an off-white solid. LCMS (Method E): Rt=0.460 min, [M+H]⁺=733.3.

### Step 5: Synthesis of Intermediate 1-10

To a solution of Intermediate 1-8 (20 g, 86.56 mmol, 1 eq), Intermediate 1-9 (19.47 g, 129.84 mmol, 1.5 eq), ditert-butyl(cyclopenta-1,4-dien-1-yl)phosphane;dichloropalladium;iron (2.82 g, 4.33 mmol, 0.05 eq) in dioxane (200 mL) and H₂O (20 mL) was added K₃PO₄ (55.12 g, 259.69 mmol, 3 eq). Then the mixture was stirred at 60° C. for 1 hr under N₂. The reaction mixture was poured into water (200 mL) and extracted with EA (200 mL*2), the organic layer was washed with brine (200 mL) and dried over Na₂SO₄. The combined organics were concentrated to give the crude product. The crude product was purified by flash silica gel chromatography (SiO₂, PE:EA=10:1 to 1:1) to give Intermediate 1-10 (16.5 g, 64.25 mmol, 74.22% yield, 99.8% purity) as off-white solid. LCMS (Method E): Rt=0.469 min, [M+H]⁺=257.2.

### Step 6: Synthesis of Intermediate 1-11

To a solution of Intermediate 1-10 (16.5 g, 64.38 mmol, 1 eq) and pyridine (15.28 g, 193.13 mmol, 15.59 mL, 3 eq) in DCM (160 mL) was added acetyl chloride (7.33 g, 93.35 mmol, 6.64 mL, 1.45 eq) at 0° C. Then the mixture was stirred at 0° C. for 1 hr. The reaction mixture was poured into water (300 mL) and extracted with DCM (300 mL*2), the organic layer was washed with brine (300 mL) and dried over Na₂SO₄. The combined organics were concentrated to give the crude product. The crude product was triturated with PE:EA=3:1 (5 mL) at 25° C. for 30 min to give Intermediate 1-11 (19 g, 63.15 mmol, 98.10% yield, 99.16% purity) as white solid. LCMS (Method E): Rt=0.542 min, [M+H]⁺=299.2.

### Step 7: Synthesis of Intermediate 1-12

To a solution of Intermediate 1-11 (19 g, 63.69 mmol, 1 eq) in MeOH (190 mL) and H₂O (190 mL) was added LiOH·H₂O (13.36 g, 318.43 mmol, 5 eq). Then the mixture was stirred at 25° C. for 1 hr. The reaction mixture was poured into water (500 mL) and adjusted to pH=2 with 1N HCl, and extracted with EA (500 mL*2). The organic layer was washed with brine (500 mL) and dried over Na₂SO₄. The combined organics were concentrated to give the crude product as an off-white solid. Intermediate 1-12 (16 g, 56.28 mmol, 88.37% yield, 100% purity) was obtained as an off-white solid. LCMS (Method E): Rt=0.461 min, [M+H]⁺=285.1.

### Step 8: Synthesis of Intermediate 1-13

To a solution of Intermediate 1-12 (4 g, 14.07 mmol, 1 eq), EDCI (5.39 g, 28.14 mmol, 2 eq), HOAt (957.49 mg, 7.03 mmol, 984.06 μL, 0.5 eq), and NMM (7.12 g, 70.35 mmol, 7.73 mL, 5 eq) in DCM (40 mL) was added Intermediate 1-7 (10.82 g, 14.07 mmol, 1 eq, HCl salt). Then the mixture was stirred at 25° C. for 1 hr. The reaction mixture was poured into water (30 mL) and extracted with DCM (30 mL*2), the organic layer was washed with brine (30 mL) and dried over Na₂SO₄. The combined organics were concentrated to give the crude product as a yellow oil. The crude product was used for the next step without further purification. Intermediate 1-13 (13 g, 8.82 mmol, 62.70% yield, 67.8% purity) was obtained as a yellow oil. LCMS (Method E): Rt=0.645 min, [M+H]⁺=999.5.

### Step 9: Synthesis of I-403

To a solution of Intermediate 1-13 (13 g, 13.01 mmol, 1 eq) in DCM (130 mL) was added piperidine (5.54 g, 65.06 mmol, 6.42 mL, 5 eq). Then the mixture was stirred at 25° C. for 1 hr. The reaction mixture was filtered to give the filtrate. The filtrate was purified by prep-HPLC (column: Kromasil Eternity XT 250*80 mm*10 um; mobile phase: [water (NH₄HCO₃)-ACN]; gradient: 28%-58% B over 20 min) and then 0.8 g of TFA was added to give I-403 (5.89 g, 6.60 mmol, 50.71% yield, 99.80% purity, TFA salt) as off-white solid, after lyophilization. LCMS (Method E): Rt=0.484 min, [M+H]⁺=777.4. HPLC: Rt=11.423 min. ¹H NMR (400 MHz, DMSO-d₆) δ=12.60 (s, 1H), 12.11 (br d, J=4.0 Hz, 1H), 9.37-9.09 (m, 2H), 8.60 (br d, J=3.2 Hz, 1H), 8.36-8.21 (m, 1H), 7.97-7.81 (m, 3H), 7.55-7.43 (m, 4H), 7.35 (br s, 2H), 7.24 (br d, J=3.6 Hz, 1H), 4.33 (br s, 2H), 4.05-3.81 (m, 2H), 3.77-3.48 (m, 9H), 3.47-3.38 (m, 2H), 3.29 (br s, 1H), 3.20 (br d, J=20.0 Hz, 2H), 3.04 (br d, J=4.8 Hz, 2H), 2.76-2.64 (m, 2H), 2.19 (d, J=2.8 Hz, 3H), 1.31-1.18 (m, 3H). ¹⁹F NMR (376 MHz, DMSO-d₆) δ=−73.45 (s, 3F), −119.67 (s, 1F).

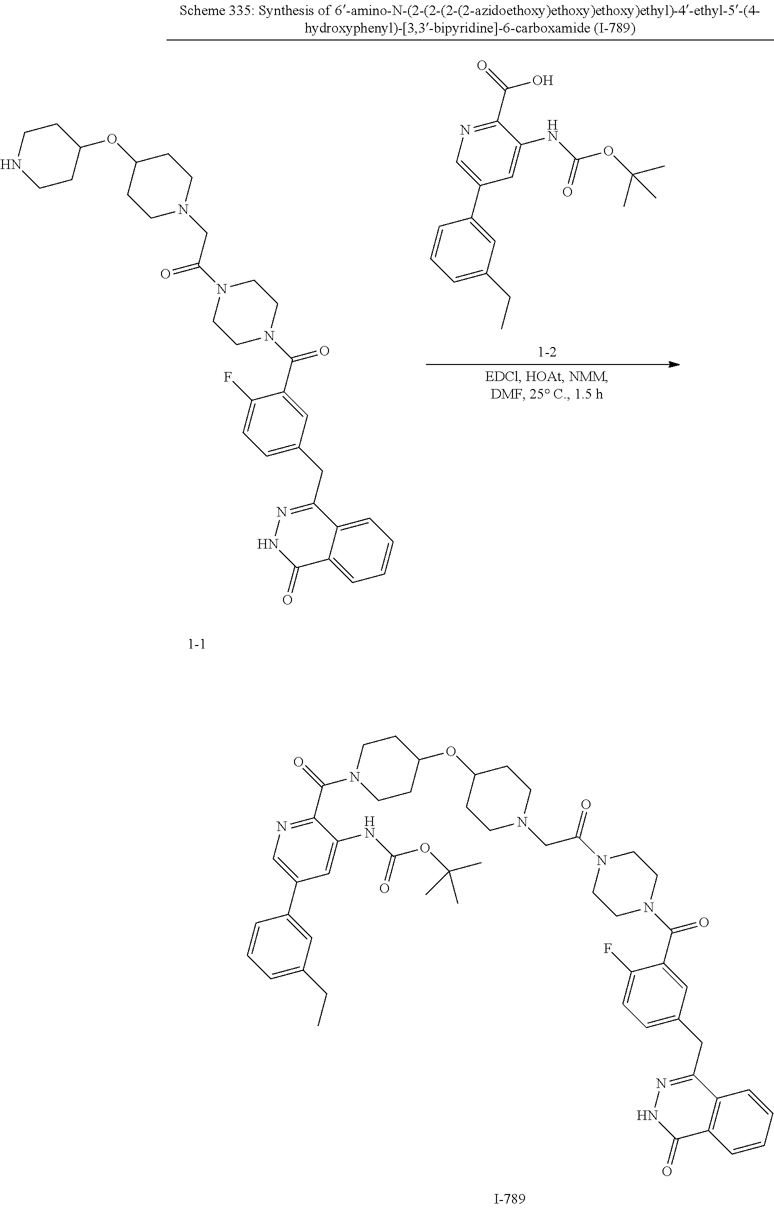 [SMILES: O=C(CN1CCC(OC2CCNCC2)CC1)N1CCN(C(=O)c2cc(Cc3n[nH]c(=O)c4ccccc34)ccc2F)CC1 ; CCc1cccc(-c2cnc(C(=O)O)c(NC(=O)OC(C)(C)C)c2)c1 ; CCc1cccc(-c2cnc(C(=O)N3CCC(OC4CCN(CC(=O)N5CCN(C(=O)c6cc(Cc7n[nH]c(=O)c8ccccc78)ccc6F)CC5)CC4)CC3)c(NC(=O)OC(C)(C)C)c2)c1]

To a solution of Intermediate 1-1 (50 mg, 84.65 μmol, 1 eq) and Intermediate 1-2 (28.98 mg, 84.65 μmol, 1 eq) in DMF (0.5 mL) was added EDCI (32.45 mg, 169.29 μmol, 2 eq) and HOAt (11.52 mg, 84.65 μmol, 11.84 μL, 1 eq) and NMM (42.81 mg, 423.24 μmol, 46.53 μL, 5 eq). The mixture was stirred at 25° C. for 1 hr. To the reaction mixture was added H₂O (2 mL) and then the reaction mixture was extracted with DCM (2 mL*3), the combined organic phase was washed, dried by Na₂SO₄, filtered and concentrated. The residue was purified by prep-HPLC (column: Phenomenex luna C18 150*25 mm*10 um; mobile phase: [water (FA)-ACN]; gradient: 28%-58% B over 10 min) and the eluent was concentrated to remove ACN and lyophilized to give product. I-789 (8.84 mg, 9.55 μmol, 11.28% yield, 98.817% purity) was obtained as a white solid. LCMS (Method D): Retention time: 0.417 min, (M+H)=915.5. ¹H NMR (400 MHz, METHANOL-d4) δ=8.54 (d, J=1.6 Hz, 1H), 8.45-8.32 (m, 2H), 7.96-7.94 (m, 1H), 7.91-7.80 (m, 2H), 7.55-7.46 (m, 3H), 7.46-7.35 (m, 2H), 7.31 (d, J=7.6 Hz, 1H), 7.17 (t, J=8.8 Hz, 1H), 4.59 (s, 2H), 4.39 (s, 2H), 4.12-4.00 (m, 1H), 3.83-3.48 (m, 11H), 2.92-2.69 (m, 4H), 2.53-2.21 (m, 2H), 2.00-1.85 (m, 4H), 1.74-1.60 (m, 4H), 1.54 (s, 9H), 1.30 (t, J=7.6 Hz, 5H).

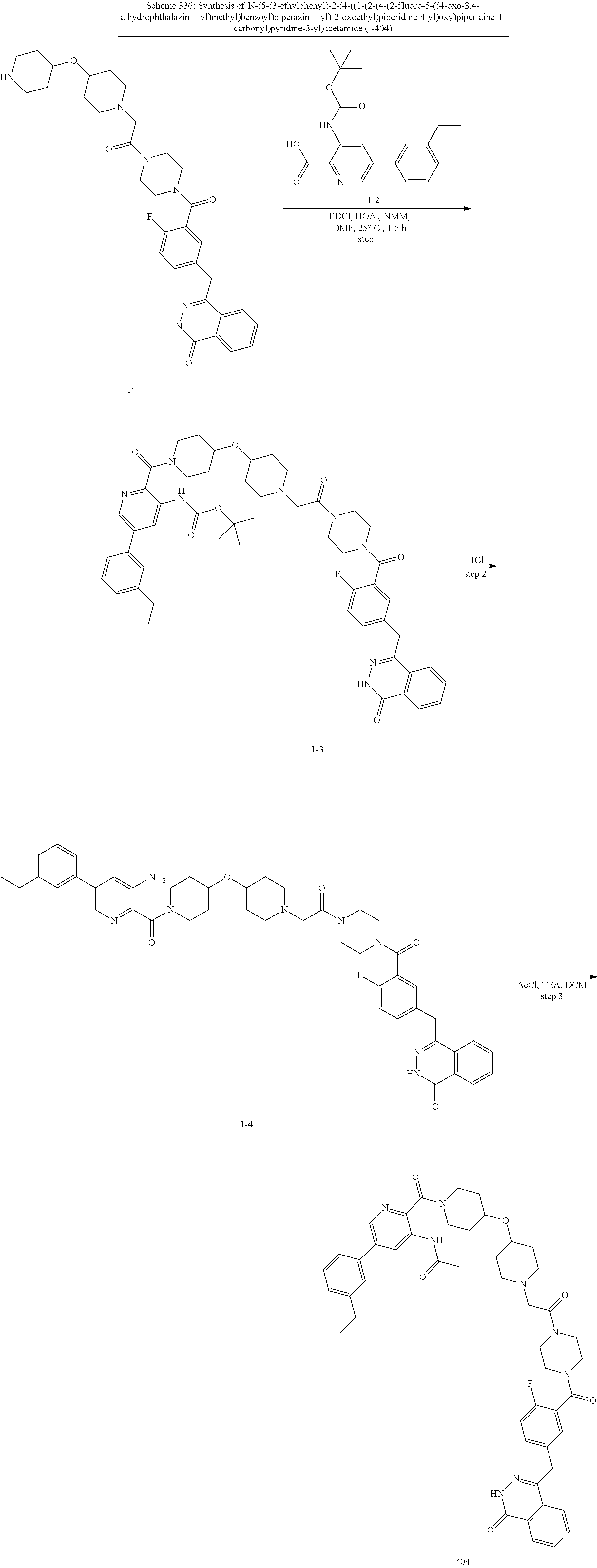 [SMILES: O=C(CN1CCC(OC2CCNCC2)CC1)N1CCN(C(=O)c2cc(Cc3n[nH]c(=O)c4ccccc34)ccc2F)CC1 ; CCc1cccc(-c2cnc(C(=O)O)c(NC(=O)OC(C)(C)C)c2)c1 ; CCc1cccc(-c2cnc(C(=O)N3CCC(OC4CCN(CC(=O)N5CCN(C(=O)c6cc(Cc7n[nH]c(=O)c8ccccc78)ccc6F)CC5)CC4)CC3)c(NC(=O)OC(C)(C)C)c2)c1 ; Cl ; CCc1cccc(-c2cnc(C(=O)N3CCC(OC4CCN(CC(=O)N5CCN(C(=O)c6cc(Cc7n[nH]c(=O)c8ccccc78)ccc6F)CC5)CC4)CC3)c(N)c2)c1 ; CCc1cccc(-c2cnc(C(=O)N3CCC(OC4CCN(CC(=O)N5CCN(C(=O)c6cc(Cc7n[nH]c(=O)c8ccccc78)ccc6F)CC5)CC4)CC3)c(NC(C)=O)c2)c1]

### Step 1: Synthesis of Intermediate 1-3

To a solution of Intermediate 1-1 (150 mg, 253.94 μmol, 1 eq) and Intermediate 1-2 (86.95 mg, 253.94 μmol, 1 eq) in DMF (1.5 mL) was added EDCI (97.36 mg, 507.88 μmol, 2 eq) and HOAt (34.56 mg, 253.94 Mol, 35.52 μL, 1 eq) and NMM (128.43 mg, 1.27 mmol, 139.59 μL, 5 eq). The mixture was stirred at 25° C. for 1 hr. To the reaction mixture was added H₂O (10 ml). The reaction mixture was then filtered and concentrated under reduced pressure to give a residue. The crude was used in the next step. Intermediate 1-3 (120 mg, 131.14 Mol, 51.64% yield) was obtained as a white solid. LCMS (Method D): Retention time: 0.412 min, (M+H)=915.5.

### Step 2: Synthesis of Intermediate 1-4

To a solution of Intermediate 1-3 (70 mg, 76.50 μmol, 1 eq) in DCM (0.6 mL) was added TFA (307.00 mg, 2.69 mmol, 0.2 mL, 35.20 eq). The mixture was stirred at 25° C. for 1 hr. The reaction mixture was concentrated under reduced pressure to give a residue. The crude was used in the next step. Intermediate 1-4 (40 mg, 43.06 μmol, 56.29% yield, TFA salt) was obtained as a white solid. LCMS (Method D): Retention time: 0.356 min, (M+H)=815.4.

### Step 3: Synthesis of I-404

To a solution of Intermediate 1-4 (30 mg, 36.81 μmol, 1 eq) in DCM (0.3 mL) was added DIEA (9.52 mg, 73.62 μmol, 12.82 μL, 2 eq) and acetyl chloride (3.47 mg, 44.17 μmol, 3.14 μL, 1.2 eq). The mixture was stirred at 0° C. for 1 hr. The reaction mixture was diluted with MeOH (2 mL). The crude product was purified by reversed-phase HPLC (0.1% TFA condition), and the eluent was concentrated to remove ACN and lyophilized to give product. I-404 (16.6 mg, 16.94 μmol, 46.02% yield, 99.104% purity, TFA salt) was obtained as a white solid LCMS (Method D): Retention time: 0.359 min, (M+H)=857.4. ¹H NMR (400 MHz, METHANOL-d4) δ=8.63 (s, 1H), 8.44-8.33 (m, 1H), 8.19 (s, 1H), 7.94 (d, J=5.2 Hz, 1H), 7.91-7.77 (m, 2H), 7.55-7.47 (m, 3H), 7.46-7.29 (m, 3H), 7.18 (t, J=9.2 Hz, 1H), 4.39 (s, 2H), 4.31 (s, 1H), 4.23 (s, 1H), 4.07-3.91 (m, 2H), 3.86-3.43 (m, 11H), 3.36 (s, 5H), 3.20-3.04 (m, 1H), 2.77-2.71 (m, 2H), 2.23-1.83 (m, 8H), 1.80-1.64 (m, 2H), 1.29 (t, J=7.6 Hz, 3H).

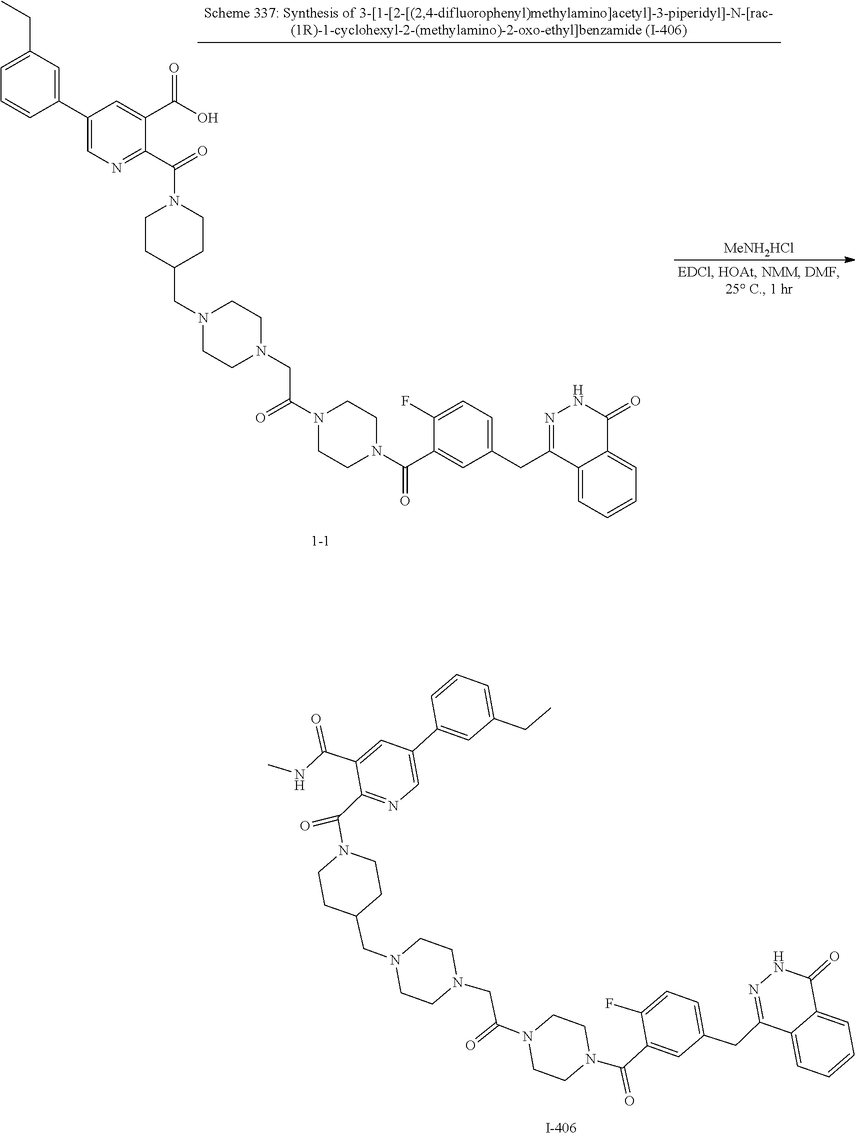 [SMILES: "MeNH2HCl" ; CCc1cccc(-c2cnc(C(=O)N3CCC(CN4CCN(CC(=O)N5CCN(C(=O)c6cc(Cc7n[nH]c(=O)c8ccccc78)ccc6F)CC5)CC4)CC3)c(C(=O)O)c2)c1 ; CCc1cccc(-c2cnc(C(=O)N3CCC(CN4CCN(CC(=O)N5CCN(C(=O)c6cc(Cc7n[nH]c(=O)c8ccccc78)ccc6F)CC5)CC4)CC3)c(C(=O)NC)c2)c1]

To a solution of Intermediate 1-1 (30 mg, 35.59 μmol, 1 eq) in DMF (0.3 mL) was added MeNH₂·HCl (2.64 mg, 39.15 μmol, 1.1 eq), EDCI (20.47 mg, 106.77 μmol, 3 eq), HOAt (4.84 mg, 35.59 mol, 4.98 μL, 1 eq) and NMM (18.00 mg, 177.95 μmol, 19.56 μL, 5 eq). The mixture was stirred at 25° C. for 1 hr. The reaction mixture was diluted with H₂O (0.5 mL) and extracted with EA (1 mL*2). The combined organic layers were washed with brine (2 mL*1), dried over Na₂SO₄, filtered and concentrated under reduced pressure to give a residue. The crude product was purified by prep-HPLC (column: Phenomenex luna C18 150*25 mm*10 um; mobile phase: [water (FA)-ACN]; gradient: 12%-42% B over 8 min). The eluent was concentrated under reduced pressure to remove ACN and then lyophilized to give I-406 (8.26 mg, 9.06 μmol, 25.46% yield, 98.933% purity, FA salt) as a white solid. LCMS (Method D): Retention time: 0.362 min, (M+H)=856.3. ¹H NMR (400 MHz, METHANOL-d₄) δ=8.93-8.85 (m, 1H), 8.43-8.28 (m, 2H), 8.02-7.78 (m, 3H), 7.63-7.53 (m, 2H), 7.52-7.29 (m, 4H), 7.20-7.12 (m, 1H), 4.71-4.51 (m, 2H), 4.38 (s, 2H), 3.86-3.62 (m, 4H), 3.59-3.47 (m, 2H), 3.46-3.33 (m, 2H), 3.30-3.20 (m, 2H), 3.17-3.06 (m, 1H), 2.97-2.84 (m, 4H), 2.81-2.42 (m, 10H), 2.39-2.26 (m, 2H), 1.98-1.84 (m, 2H), 1.74-1.62 (m, 1H), 1.46-1.23 (m, 5H). ¹⁹FNMR (376 MHz, MeOD-d₆) δ=−120.682.

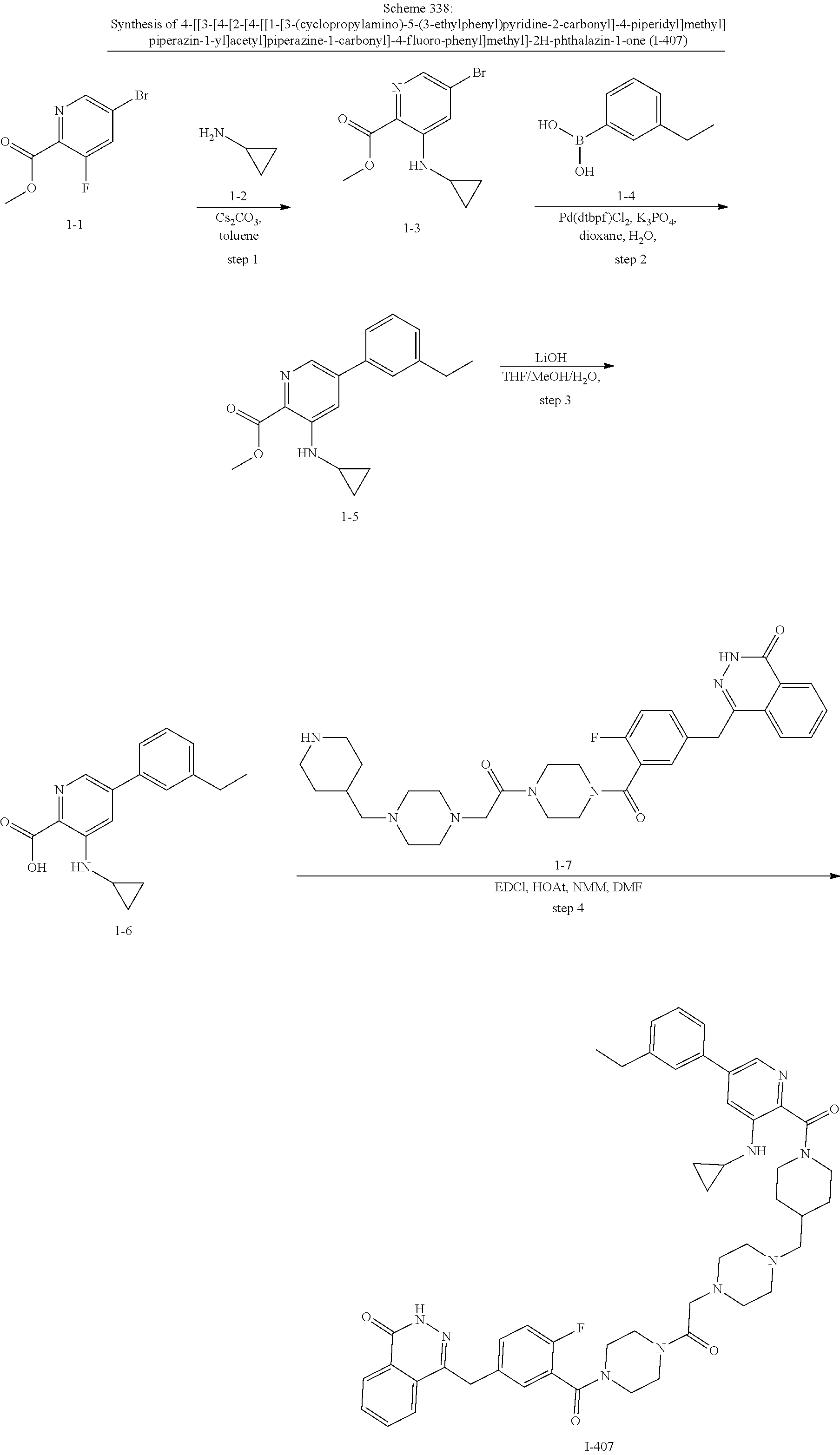

### Step 1: Synthesis of Intermediate 1-3

To a solution of intermediate 1-1 (500 mg, 2.14 mmol, 1 eq) and intermediate 1-2 (134.18 mg, 2.35 mmol, 162.84 μL, 1.1 eq) in DMF (5 mL) was added Cs₂CO₃ (2.09 g, 6.41 mmol, 3 eq), then the mixture was stirred at 100° C. for 1 hour under N₂. The reaction mixture was diluted with a saturated NaCl solution (30 mL) and extracted with EA (20 mL*2). The combined organic layers were concentrated under reduced pressure to give a residue. The residue was purified by flash silica gel chromatography (ISCO; 20 g SepaFlash Silica Flash Column, Eluent of 0-30% Ethyl acetate/Petroleum ether gradient @80 mL/min). The eluent was concentrated under reduced pressure to give the product. Intermediate 1-3 (220 mg, 660.54 mol, 30.92% yield, 81.4% purity) was obtained as a yellow solid. LCMS (Method D): Rt=0.403 min, (M+H)=270.9.

### Step 2: Synthesis of Intermediate 1-5

To a mixture of intermediate 1-3 (200 mg, 737.71 μmol, 1 eq) and intermediate 1-4 (165.96 mg, 1.11 mmol, 1.5 eq) in dioxane (2 mL) and H₂O (0.4 mL) was added K₃PO₄ (469.77 mg, 2.21 mmol, 3 eq) and ditert-butyl(cyclopentyl)phosphane; dichloropalladium; iron (48.08 mg, 73.77 μmol, 0.1 eq). The mixture was stirred at 80° C. for 1 hr under N₂. The reaction mixture was diluted with water (40 mL) and extracted with EA (30 mL*2). The combined organic layers were concentrated under reduced pressure to give a residue. The residue was purified by flash silica gel chromatography (ISCO; 20 g SepaFlash Silica Flash Column, Eluent of 0˜10% Dichloromethane: Methanol@60 mL/min). The eluent was concentrated under reduced pressure to give the product. Intermediate 1-5 (180 mg, 607.36 μmol, 82.33% yield) was obtained as a white solid. LCMS (Method D): Rt=0.438 min, (M+H)=297.2.

### Step 3: Synthesis of Intermediate 1-6

To a mixture of intermediate 1-5 (150 mg, 506.14 μmol, 1 eq) in THF (2 mL), MeOH (1 mL) and H₂O (0.5 mL) was added LiOH·H₂O (63.72 mg, 1.52 mmol, 3 eq), then the mixture was stirred at 20° C. for 0.5 hour. The reaction mixture was concentrated under reduced pressure to remove THF and MeOH and 1 M HCl was added (about 1 mL) to adjust the pH to 3-4. The reaction mixture was extracted with EA (10 mL*3). The combined organic layers were concentrated under reduced pressure to give a residue. Intermediate 1-6 (130 mg, 460.44 μmol, 90.97% yield) was obtained as a white solid. LCMS (Method D): Rt=0.387 min, (M+H)=283.1.

### Step 4: Synthesis of I-407

To a solution of intermediate 1-6 (100 mg, 354.19 gmol, 1 eq) and intermediate 1-7 (208.87 mg, 354.19 μmol, 1 eq) in DMF (1 mL) was added EDCI (203.69 mg, 1.06 mmol, 3 eq), HOAt (48.21 mg, 354.19 μmol, 49.55 μL, 1 eq) and NMM (179.13 mg, 1.77 mmol, 194.70 μL, 5 eq), then the mixture was stirred at 40° C. for 1 hour. The reaction mixture was concentrated under reduced pressure to give a residue. The crude product was purified by reversed-phase HPLC (0.1% FA condition). The eluent was concentrated under reduced pressure to remove ACN and then lyophilized to give the product. I-407 (17.08 mg, 18.98 mol, 5.36% yield, 100.000% purity, FA salt) was obtained as a white solid. LCMS (Method D): Rt=0.389 min, (M+H)=854.5. ¹H NMR (400 MHz, METHANOL-d4) δ=8.49 (s, 1H), 8.39 (d, J=7.6 Hz, 1H), 8.10 (d, J=1.6 Hz, 1H), 8.00-7.95 (m, 1H), 7.92-7.83 (m, 2H), 7.69 (d, J=1.6 Hz, 1H), 7.55-7.47 (m, 3H), 7.45-7.37 (m, 2H), 7.31 (d, J=7.2 Hz, 1H), 7.18 (t, J=8.9 Hz, 1H), 4.70-4.59 (m, 2H), 4.40 (s, 2H), 3.83-3.68 (m, 5H), 3.57-3.49 (m, 2H), 3.38 (s, 2H), 3.31 (s, 1H), 3.16-3.08 (m, 1H), 2.94-2.87 (m, 1H), 2.77-2.73 (m, 3H), 2.57 (s, 8H), 2.40-2.37 (m, 2H), 2.00-1.89 (m, 2H), 1.78-1.73 (m, 1H), 1.35-1.22 (m, 5H), 0.90-0.84 (m, 2H), 0.59-0.52 (m, 2H). ¹⁹F NMR (400 MHz, METHANOL-d4) δ=−120.787.

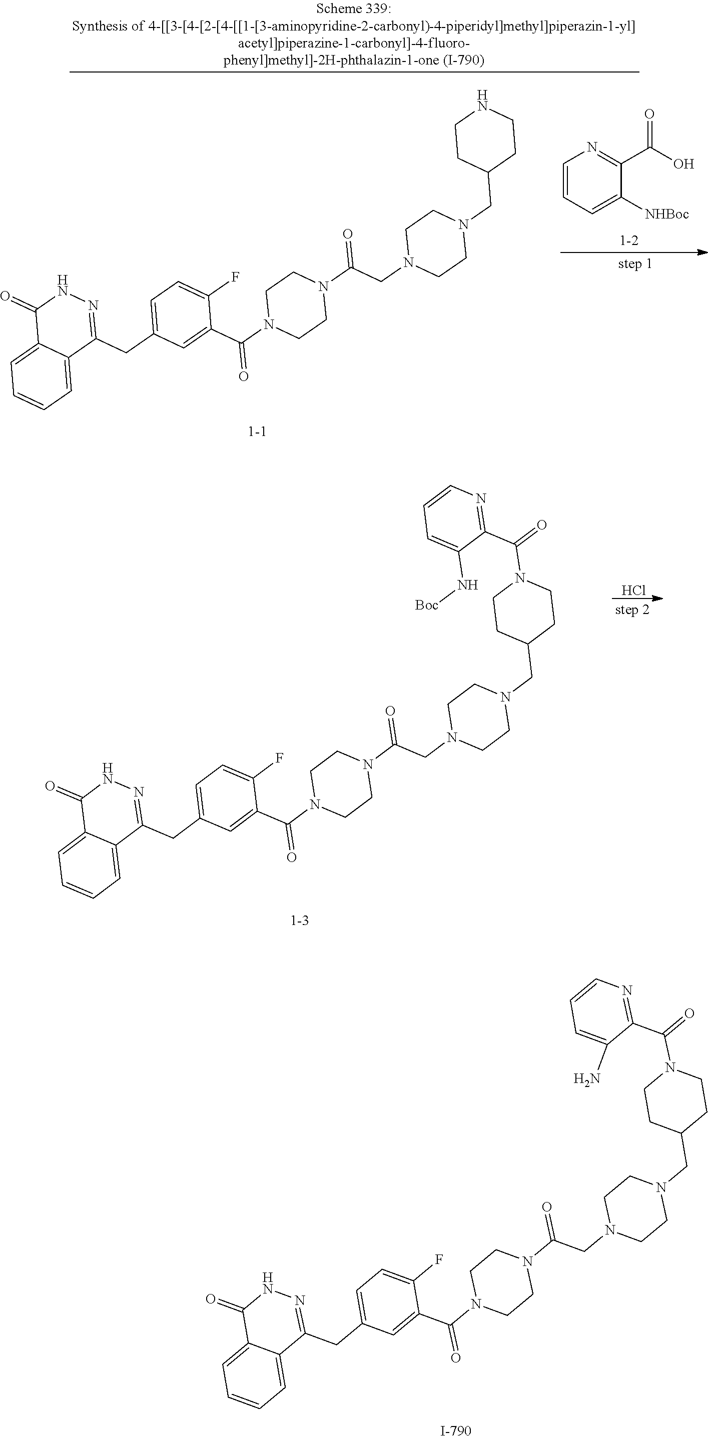 [SMILES: O=C(CN1CCN(CC2CCNCC2)CC1)N1CCN(C(=O)c2cc(Cc3n[nH]c(=O)c4ccccc34)ccc2F)CC1 ; CC(C)(C)OC(=O)Nc1cccnc1C(=O)O ; CC(C)(C)OC(=O)Nc1cccnc1C(=O)N1CCC(CN2CCN(CC(=O)N3CCN(C(=O)c4cc(Cc5n[nH]c(=O)c6ccccc56)ccc4F)CC3)CC2)CC1 ; Cl ; Nc1cccnc1C(=O)N1CCC(CN2CCN(CC(=O)N3CCN(C(=O)c4cc(Cc5n[nH]c(=O)c6ccccc56)ccc4F)CC3)CC2)CC1]

### Step 1: Synthesis of Intermediate 1-3

To a solution of Intermediate 1-2 (40.40 mg, 169.58 μmol, 1 eq) in DMF (1 mL) was added NMM (85.76 mg, 847.89 μmol, 93.22 μL, 5 eq) and EDCI (162.54 mg, 847.89 μmol, 5 eq), HOAt (46.16 mg, 339.15 μmol, 47.44 μL, 2 eq), Intermediate 1-1 (100 mg, 169.58 μmol, 1 eq). The mixture was stirred at 25° C. for 2 hr. The mixture was washed with water (1 mL) and extracted with EA (1 mL*3), the combined organic phase was dried by Na₂SO₄, and concentrated under reduced pressure to give a residue. The crude was purified by prep-HPLC (column: Phenomenex luna C18 150*25 mm*10 um; mobile phase: [water(FA)-ACN]; gradient: 5%-35% B over 8 min) the eluent was concentrated to remove ACN and lyophilized to give product. Intermediate 1-3 (100 mg, 114.12 μmol, 67.30% yield, 92.428% purity) was obtained as a yellow solid. LCMS (Method D): Retention time: 0.320 min, (M+H)=810.4.

### Step 2: Synthesis of I-790

To a solution of Intermediate 1-3 (100 mg, 123.47 μmol, 1 eq) was added HCl/dioxane (2 M, 1 mL) and the mixture was stirred at 25° C. for 1 hr. The reaction mixture was concentrated under reduced pressure to give a residue. The crude was purified by prep-HPLC (column: Phenomenex luna C18 150*25 mm*10 um; mobile phase: [water(FA)-ACN]; gradient: 0%-30% B over 10 min) and the eluent was concentrated to remove ACN and lyophilized to give product. I-790 (36 mg, 47.22 μmol, 38.24% yield, 99.138% purity, FA salt) was obtained as a white solid. LCMS (Method D): Retention time: 0.263 min, (M+H)=710.3. ¹H NMR (400 MHz, METHANOL-d4) δ=8.43-8.32 (m, 2H), 7.99-7.91 (m, 1H), 7.91-7.78 (m, 3H), 7.50 (d, J=5.2 Hz, 1H), 7.40-7.33 (m, 1H), 7.25-7.12 (m, 3H), 4.66 (d, J=12.0 Hz, 1H), 4.38 (s, 2H), 3.82-3.72 (m, 2H), 3.66 (s, 3H), 3.57-3.44 (m, 4H), 3.34 (s, 1H), 3.13 (t, J=12.4 Hz, 1H), 3.01 (s, 4H), 2.93-2.78 (m, 5H), 2.71 (d, J=6.4 Hz, 2H), 2.04 (s, 1H), 1.91 (d, J=11.6 Hz, 1H), 1.74 (d, J=11.6 Hz, 1H), 1.31 (d, J=10.4 Hz, 2H).

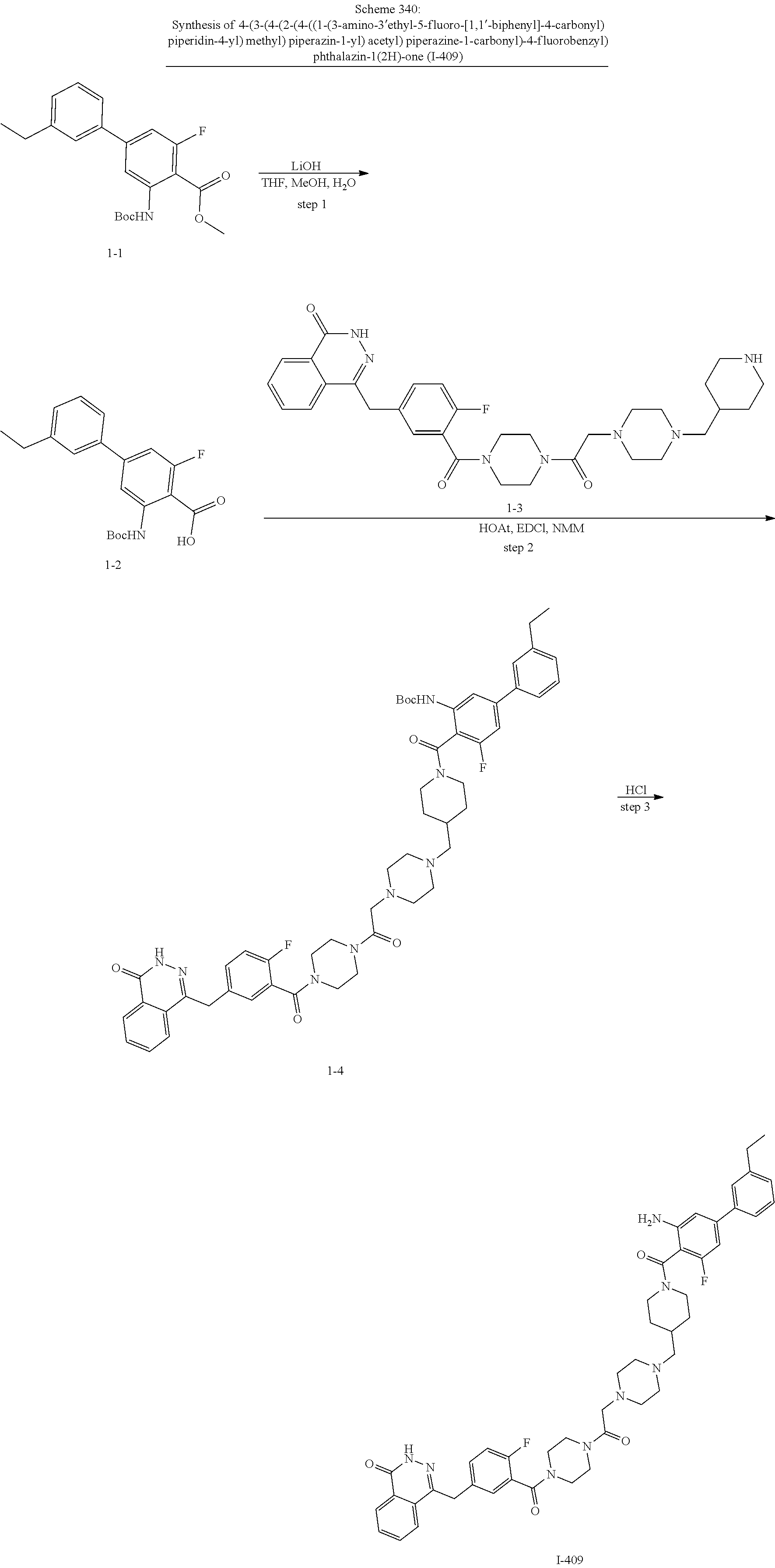

### Step 1: Synthesis of Intermediate 1-2

To a solution of Intermediate 1-1 (40 mg, 107.12 μmol, 1 eq) in THF (0.1 mL) and MeOH (0.1 mL) and H₂O (0.1 mL) was added LiOH·H₂O (8.99 mg, 214.24 μmol, 2 eq). The mixture was stirred at 25° C. for 1 hr. To the reaction mixture was added H₂O (2 mL) and then it was extracted with DCM (2 mL*3), the combined organic phase was washed, dried by Na₂SO₄, filtered and concentrated. The crude was used in the next step. Intermediate 1-2 (46 mg, crude) was obtained as a white solid. LCMS (Method D): Retention time: 0.477 min, (M+H)=382.1.

### Step 2: Synthesis of Intermediate 1-4

To a solution of Intermediate 1-2 (45.92 mg, 127.76 μmol, 1 eq) and Intermediate 1-3 (80 mg, 127.76 μmol, 1 eq, HCl salt) in DMF (0.8 mL) was added EDCI (48.98 mg, 255.52 μmol, 2 eq) and HOAt (17.39 mg, 127.76 μmol, 17.87 μL, 1 eq) and NMM (64.61 mg, 638.81 μmol, 70.23 μL, 5 eq). The mixture was stirred at 25° C. for 1 hr. The reaction mixture was diluted with MeOH (1 mL). The crude product was purified by reversed-phase HPLC (0.1% FA condition), and the eluent was concentrated to remove ACN and lyophilized to give product. Intermediate 1-4 (50 mg, 50.48 μmol, 39.51% yield, 94% purity) was obtained as a white solid. LCMS (Method D): Retention time: 0.411 min, (M+H)=931.5.

### Step 3: Synthesis of I-409

To a solution of Intermediate 1-4 (40 mg, 42.96 μmol, 1 eq) in DCM (0.2 mL) was added HCl/dioxane (2 M, 0.2 mL, 9.31 eq). The mixture was stirred at 25° C. for 1 hr. The reaction mixture was diluted with MeOH (1 mL). The crude product was purified by reversed-phase HPLC (0.1% TFA condition) and the eluent was concentrated to remove ACN and lyophilized to give product. I-409 (25.6 mg, 31.28 μmol, 72.80% yield, 99.321% purity) was obtained as a yellow solid. LCMS (Method D): Retention time: 0.374 min, (M+H)=831.4. ¹H NMR (400 MHz, METHANOL-d4) δ=8.37 (d, J=7.2 Hz, 1H), 8.04-7.76 (m, 3H), 7.51 (d, J=4.0 Hz, 1H), 7.45-7.29 (m, 4H), 7.26-7.12 (m, 2H), 6.91 (s, 1H), 6.74-6.70 (m, 1H), 4.75 (d, J=13.2 Hz, 1H), 4.39 (s, 2H), 4.05-4.01 (m, 2H), 3.85-3.68 (m, 4H), 3.62-3.51 (m, 3H), 3.49-3.32 (m, 8H), 3.28-3.15 (m, 2H), 3.09-2.89 (m, 3H), 2.73-2.67 (m, 2H), 2.25-2.12 (m, 1H), 2.04-1.84 (m, 2H), 1.42-1.25 (m, 5H).

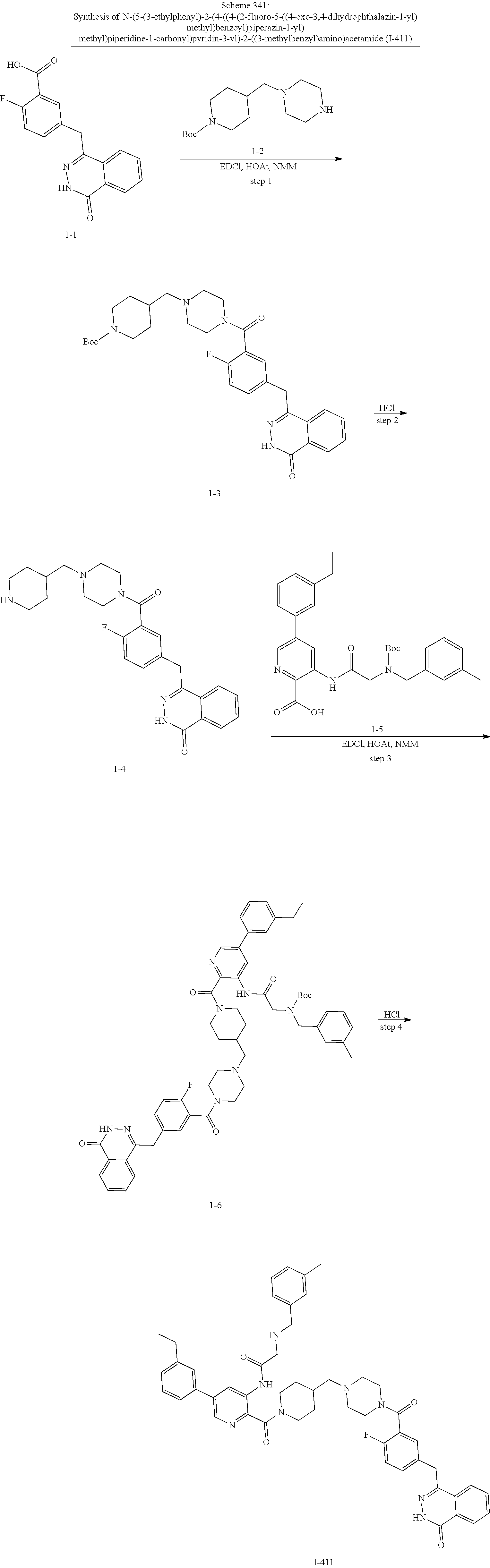

### Step 1: Synthesis of Intermediate 1-3

To a solution of Intermediate 1-1 (200 mg, 670.54 μmol, 1 eq) and Intermediate 1-2 (190.04 mg, 670.54 μmol, 1 eq) in DMF (2 mL) was added EDCI (257.09 mg, 1.34 mmol, 2 eq) and HOAt (91.27 mg, 670.54 μmol, 93.80 μL, 1 eq) and NMM (339.11 mg, 3.35 mmol, 368.60 μL, 5 eq). The mixture was stirred at 25° C. for 1 hr. To the reaction mixture was added to H₂O (10 mL). The reaction mixture was filtered and was concentrated under reduced pressure to give a residue. The crude was used in the next step. Intermediate 1-3 (330 mg, 585.46 μmol, 87.31% yield) was obtained as a white solid. LCMS (Method D): Retention time: 0.302 min, (M+H)=564.2.

### Step 2: Synthesis of Intermediate 1-4

A solution of Intermediate 1-3 (300 mg, 532.23 μmol, 1 eq) in HCl/dioxane (2 M, 3.00 mL, 11.27 eq) was stirred at 25° C. for 1 hr. The reaction mixture was concentrated under reduced pressure to give a residue. The crude was used in the next step directly. Intermediate 1-4 (300 mg, crude, HCl salt) was obtained as a white solid. LCMS (Method D): Retention time: 0.190 min, (M+H)=464.2.

### Step 3: Synthesis of Intermediate 1-6

To a solution of Intermediate 1-4 (270 mg, 539.99 μmol, 1 eq, HCl salt) and Intermediate 1-5 (299.13 mg, 593.99 μmol, 1.1 eq) in DMF (3 mL) was added EDCI (207.03 mg, 1.08 mmol, 2 eq) and HOAt (73.50 mg, 539.99 μmol, 75.54 μL, 1 eq) and NMM (273.09 mg, 2.70 mmol, 296.84 μL, 5 eq). The mixture was stirred at 25° C. for 1 hr. The reaction mixture was diluted with MeOH (1 mL). The crude product was purified by reversed-phase HPLC (0.1% FA condition), and the eluent was concentrated to remove ACN and lyophilized to give Intermediate 1-6 (130 mg, 136.97 μmol, 25.37% yield) as a white solid. LCMS (Method D): Retention time: 0.432 min, (M+H)=949.5.

### Step 4: Synthesis of I-411

To a solution of Intermediate 1-6 (110 mg, 115.90 μmol, 1 eq) in DCM (0.5 mL) was added HCl/dioxane (2 M, 0.5 mL, 8.63 eq). The mixture was stirred at 25° C. for 1 hr. The reaction mixture was diluted with MeOH (2 ml). The crude product was purified by reversed-phase HPLC (0.1% FA condition), the eluent was concentrated to remove ACN and lyophilized to give product. I-411 (48.22 mg, 55.69 μmol, 48.05% yield, 98.052% purity) was obtained as a white solid. LCMS (Method D): Retention time: 0.394 min, (M+H)=849.5. ¹H NMR (400 MHz, METHANOL-d4) δ=8.68-8.52 (m, 2H), 8.38-8.34 (m, 1H), 7.98-7.91 (m, 1H), 7.89-7.78 (m, 2H), 7.55-7.39 (m, 4H), 7.36-7.22 (m, 5H), 7.19-7.09 (m, 2H), 4.65 (d, J=13.2 Hz, 1H), 4.37 (s, 2H), 3.95 (s, 2H), 3.81-3.68 (m, 3H), 3.61 (s, 2H), 3.25 (s, 2H), 3.18-3.10 (m, 1H), 2.92-2.81 (m, 1H), 2.77-2.71 (m, 2H), 2.48 (s, 2H), 2.35 (s, 3H), 2.32-2.16 (m, 4H), 1.93-1.81 (m, 2H), 1.76 (d, J=14.6 Hz, 1H), 1.29 (t, J=7.6 Hz, 4H), 1.25-1.17 (m, 1H).

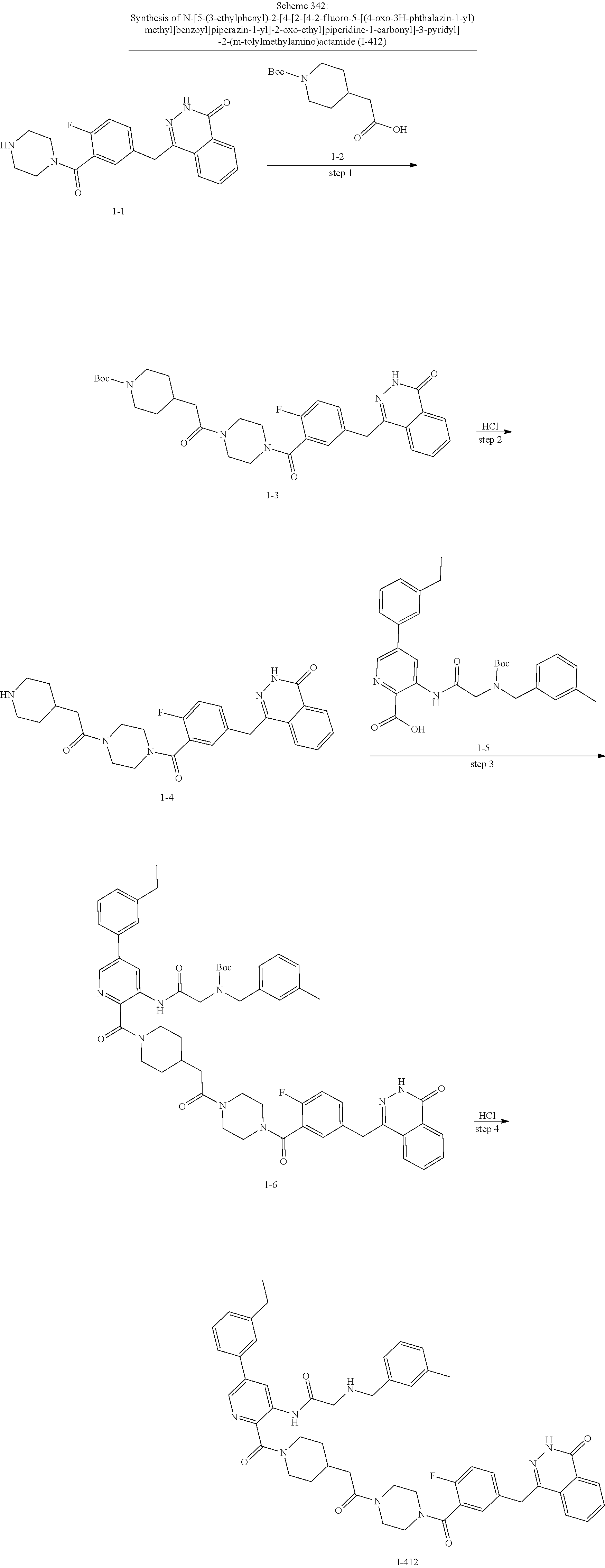

### Step 1: Synthesis of Intermediate 1-3

To a solution of Intermediate 1-2 (135.89 mg, 558.52 μmol, 1.5 eq) in DMF (1.5 mL) was added NMM (188.31 mg, 1.86 mmol, 204.68 μL, 5 eq) and EDCI (356.90 mg, 1.86 mmol, 5 eq), HOAt (101.36 mg, 744.70 μmol, 104.17 μL, 2 eq), Intermediate 1-1 (150 mg, 372.35 μmol, 1 eq, HCl salt). The mixture was stirred at 25° C. for 2 hr. The mixture was diluted with water (1.5 mL) and extracted with DCM (1.5 mL*3), the combined organic phase was dried by Na₂SO₄, and concentrated under reduced pressure to give a residue. The residue was purified by flash silica gel chromatography (ISCO; 12 g SepaFlash Silica Flash Column, Eluent of 0-50% Methanol/Dichloromethane @30 mL/min) and the eluent was concentrated to give product. Intermediate 1-3 (160 mg, 236.92 μmol, 63.63% yield, 87.61% purity) was obtained as a yellow solid. LCMS (Method D): Retention time=0.438 min, (M+H)=492.2.

### Step 2: Synthesis of Intermediate 1-4

To a solution of Intermediate 1-3 (160 mg, 270.42 μmol, 1 eq) was added HCl/dioxane (2 M, 1.6 mL) and the mixture was stirred at 25° C. for 1 hr. The reaction mixture was concentrated under reduced pressure to give a residue. ACN and H₂O were added and it was to lyophilized to give product. The crude was used for the next step. Intermediate 1-4 (160 mg, crude, HCl salt) was obtained as a white solid. LCMS (Method D): Retention time=0.288 min, (M+H)=492.1.

### Step 3: Synthesis of Intermediate 1-6

To a solution of Intermediate 1-5 (125.89 mg, 249.99 μmol, 1.2 eq) in DMF (1.1 mL) was added NMM (105.36 mg, 1.04 mmol, 114.52 μL, 5 eq) and EDCI (199.68 mg, 1.04 mmol, 5 eq), HOAt (56.71 mg, 416.65 μmol, 58.28 μL, 2 eq), Intermediate 1-4 (110 mg, 208.33 μmol, 1 eq, HCl salt). The mixture was stirred at 25° C. for 1 hr. The mixture was diluted with water (1 mL) and extracted with EA (1 mL*3), the combined organic phase was dried by Na₂SO₄, concentrated under reduced pressure to give a residue. The residue was purified by reversed-phase HPLC (0.1% of FA) and the eluent was lyophilized to give product. Intermediate 1-6 (80 mg, 81.87 μmol, 39.30% yield, 100% purity) was obtained as a white solid. LCMS (Method D): Retention time=0.546 min, (M+H)=977.4.

### Step 4: Synthesis of I-412

To a solution of Intermediate 1-6 (70 mg, 71.64 μmol, 1 eq) was added HCl/dioxane (2 M, 0.7 mL) and the mixture was stirred at 25° C. for 1 hr. The reaction mixture was concentrated under reduced pressure to give a residue. ACN and H₂O were added to the residue and it was lyophilized to give product and no further purification was performed. I-412 (61 mg, 65.00 μmol, 90.74% yield, 97.344% purity, HCl salt) was obtained as a white solid. LCMS (Method D): Retention time=0.410 min, (M+H)=877.6. ¹H NMR (400 MHz, METHANOL-d4) δ=8.70 (s, 1H), 8.39-8.33 (m, 2H), 7.98-7.92 (m, 1H), 7.90-7.80 (m, 2H), 7.55-7.48 (m, 3H), 7.44 (t, J=7.6 Hz, 1H), 7.39-7.31 (m, 5H), 7.31-7.26 (m, 1H), 7.17 (t, J=8.8 Hz, 1H), 4.66-4.58 (m, 1H), 4.38 (s, 2H), 4.29 (s, 2H), 4.03 (s, 2H), 3.81-3.71 (m, 3H), 3.70-3.64 (m, 2H), 3.57-3.45 (m, 2H), 3.36-3.32 (m, 1H), 3.19 (t, J=12.8 Hz, 1H), 2.98-2.86 (m, 1H), 2.78-2.70 (m, 2H), 2.46 (d, J=6.8 Hz, 1H), 2.39 (d, J=6.4 Hz, 4H), 2.16-2.03 (m, 1H), 1.91 (d, J=10.0 Hz, 1H), 1.72 (d, J=1.6 Hz, 1H), 1.55-1.45 (m, 1H), 1.43-1.35 (m, 1H), 1.33-1.26 (m, 4H).

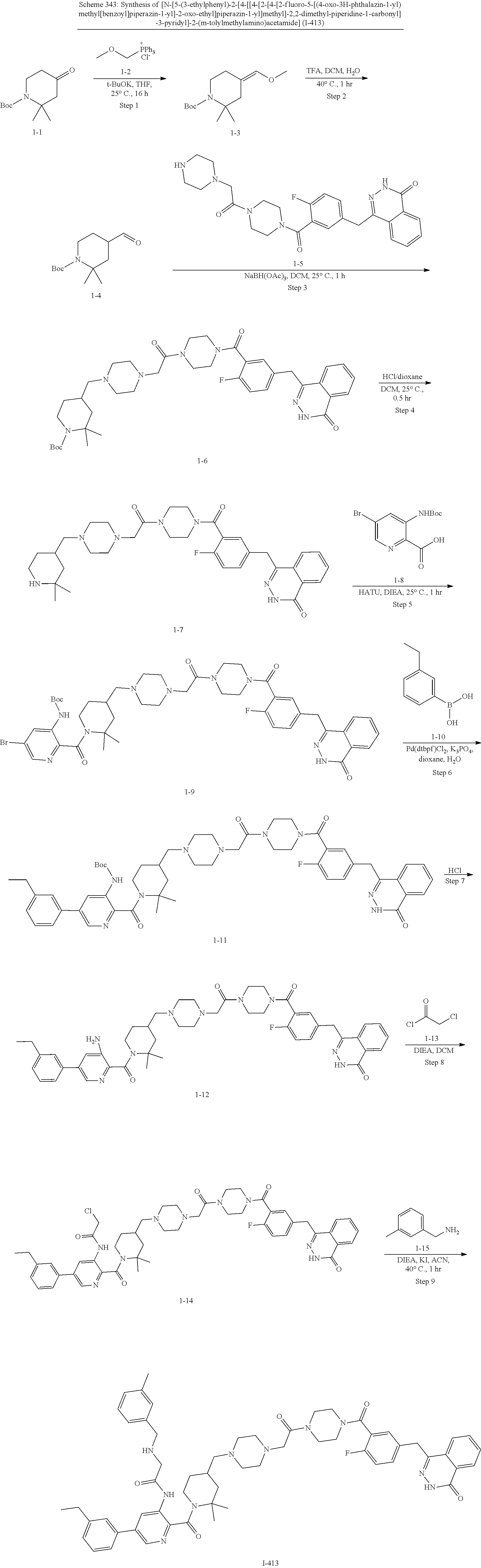

### Step 1: Synthesis of Intermediate 1-3.

To a solution of intermediate 1-2 (9.05 g, 26.40 mmol, 1.5 eq) in THF (35 mL) was added t-BuOK (2.96 g, 26.40 mmol, 1.5 eq) at 0° C. and the mixture was stirred at 0° C. for 1 h. Then intermediate 1-1 (4 g, 17.60 mmol, 1 eq) in THF (5 mL) was added and it was stirred at 25° C. for 15 h. The reaction mixture was diluted with H₂O (10 mL) and extracted with EtOAc (10 mL×3), the combined organic layers were dried over Na₂SO₄, filtered and concentrated to give a residue. The residue was purified by column chromatography (SiO₂, PE/EA=1/0 to 9/1) and concentrated to give a product. Intermediate 1-3 (3.5 g, 13.71 mmol, 77.89% yield) was obtained as colorless oil. ¹H NMR (400 MHz, METHANOL-d4) δ=6.06-5.88 (m, 1H), 3.58-3.50 (m, 5H), 2.35-2.16 (m, 4H), 1.46 (d, J=2.0 Hz, 9H), 1.38 (d, J=3.6 Hz, 6H).

### Step 2: Synthesis of Intermediate 1-4.

To a mixture of intermediate 1-3 (700 mg, 2.74 mmol, 1 eq) in DCM (6 mL) and H₂O (2 mL) was added TFA (1.88 g, 16.45 mmol, 1.22 mL, 6 eq) at 25° C., the mixture was stirred at 40° C. for 1 hr. The reaction mixture was diluted with H₂O (5 mL) and extracted with DCM (5 mL×3), the combined organic layers were dried over Na₂SO₄, filtered and concentrated to give the crude product. The crude product was used for the next step directly. Intermediate 1-4 (630 mg, 2.61 mmol, 95.23% yield) was obtained as a yellow oil.

### Step 3: Synthesis of Intermediate 1-6.

To a mixture of intermediate 1-5 (1.2 g, 2.27 mmol, 1 eq, HCl salt) in DCM (12 mL) was added TEA (22.95 mg, 226.84 μmol, 31.57 μL, 0.1 eq) and AcOH (13.62 mg, 226.84 μmol, 12.99 μL, 0.1 eq), then intermediate 1-4 (630 mg, 2.61 mmol, 1.15 eq) was added and the mixture was stirred at 25° C. for 0.5 h, then NaBH(OAc)₃ (961.54 mg, 4.54 mmol, 2 eq) was added and the mixture was stirred at 25° C. for 1 h. The reaction mixture was diluted with H O (2 mL) and extracted with DCM (2 mL×3), the combined organic layers were dried over Na₂SO₄, filtered and concentrated to give a residue. The residue was purified by column chromatography (SiO₂, EA/MeOH=1/0 to 7/3) and concentrated to give the product. Intermediate 1-6 (470 mg, 654.71 μmol, 28.86% yield) was obtained as yellow gum. LCMS (Method D): Retention time: 0.331 min, (M+1)=718.3.

### Step 4: Synthesis of Intermediate 1-7.

To a mixture of intermediate 1-6 (470 mg, 654.71 μmol, 1 eq) in DCM (2.5 mL) was added HCl/dioxane (2 M, 2.5 mL, 7.64 eq) at 25° C., then the mixture was stirred at 25° C. for 0.5 h. The reaction mixture was concentrated to give the crude product. The crude product was used for the next step directly. Intermediate 1-7 (430 mg, crude, HCl salt) was obtained as a yellow solid. LCMS (Method D): Retention time: 0.223 min, (M+1)=618.2.

### Step 5: Synthesis of Intermediate 1-9.

To a mixture of intermediate 1-7 (400 mg, 611.42 μmol, 1 eq, HCl salt) in DMF (4 mL) was added intermediate 1-8 (290.85 mg, 917.13 μmol, 1.5 eq), HATU (464.96 mg, 1.22 mmol, 2 eq) and DIEA (158.04 mg, 1.22 mmol, 213.00 μL, 2 eq) at 25° C. The mixture was stirred at 25° C. for 1 h. The reaction mixture was diluted with H₂O (10 mL), extracted with DCM (10 mL×3), the combined organic layers were dried over Na₂SO₄, filtered and concentrated to give a residue. The residue was purified by reversed-phase HPLC (0.1% FA condition) and lyophilized to give the product. Intermediate 1-9 (220 mg, 239.95 μmol, 39.24% yield) was obtained as a yellow solid. LCMS (Method D): Retention time: 0.437 min, (M+1)=916.3.

### Step 6: Synthesis of Intermediate 1-11

To a mixture of intermediate 1-9 (220 mg, 239.95 μmol, 1 eq) in dioxane (2 mL) and H₂O (0.4 mL) was added intermediate 1-10 (71.98 mg, 479.89 μmol, 2 eq), ditert-butyl(cyclopenta-1,4-dien-1-yl)phosphane;dichloropalladium;iron (15.64 mg, 23.99 μmol, 0.1 eq) and K₃PO₄ (152.80 mg, 719.84 μmol, 3 eq) at 25° C. The mixture was degassed and purged with N₂ 3 times, and then the mixture was stirred at 80° C. for 1 h under N₂ atmosphere. The reaction mixture was diluted with H₂O (2 mL) and extracted with DCM (2 mL×3), the combined organic layers were dried over Na₂SO₄, filtered and concentrated to give a residue. The residue was purified by column chromatography (SiO₂, EA/MeOH=1/0 to 1/1) and concentrated to give the product. Intermediate 1-11 (165 mg, crude) was obtained as brown gum. LCMS (Method D): Retention time: 0.490 min, (M+1)=942.5. ¹H NMR (400 MHz, METHANOL-d4) δ=8.51 (s, 1H), 8.37 (d, J=8.0 Hz, 1H), 8.25 (s, 1H), 7.98-7.92 (m, 1H), 7.91-7.79 (m, 2H), 7.56-7.45 (m, 3H), 7.44-7.33 (m, 2H), 7.30 (d, J=7.2 Hz, 1H), 7.21-7.12 (m, 1H), 4.71-4.56 (m, 3H), 4.39 (s, 2H), 3.85-3.61 (m, 4H), 3.58-3.47 (m, 3H), 3.22 (s, 2H), 3.17-3.08 (m, 4H), 2.78-2.70 (m, 2H), 2.62-2.47 (m, 6H), 2.31-2.20 (m, 2H), 2.13-2.04 (m, 1H), 1.95-1.86 (m, 1H), 1.66 (s, 3H), 1.58 (s, 3H), 1.53 (s, 9H), 1.29 (d, J=5.6 Hz, 4H).

### Step 7: Synthesis of Intermediate 1-12

To a mixture of intermediate 1-11 (145 mg, 153.91 μmol, 1 eq) in DCM (1 mL) was added HCl/dioxane (2 M, 1 mL, 12.99 eq) at 25° C. The mixture was stirred at 25° C. for 0.5 hr. The reaction mixture was concentrated to give a residue. The residue was purified by prep-HPLC (column: CD01-Phenomenex luna C18 150*25*10 um; mobile phase: [water (FA)-ACN]; gradient: 14%-44% B over 10 min) and lyophilized to give the product. Intermediate 1-12 (140 mg, crude, HCl salt) was obtained as a brown solid. LCMS (Method D): Retention time: 0.394 min, (M+1)=842.4. ¹H NMR (400 MHz, METHANOL-d4) δ=8.50 (s, 1H), 8.37 (d, J=8.0 Hz, 1H), 8.06 (d, J=1.6 Hz, 1H), 7.99-7.92 (m, 1H), 7.91-7.80 (m, 2H), 7.52-7.46 (m, 1H), 7.46-7.34 (m, 5H), 7.26 (d, J=7.2 Hz, 1H), 7.16 (t, J=8.8 Hz, 1H), 4.39 (s, 2H), 3.83-3.77 (m, 1H), 3.77-3.65 (m, 3H), 3.59-3.46 (m, 4H), 3.26 (s, 2H), 3.22-3.11 (m, 2H), 2.79-2.67 (m, 3H), 2.66-2.51 (m, 7H), 2.36-2.27 (m, 2H), 2.14-2.06 (m, 1H), 1.92-1.83 (m, 1H), 1.67 (s, 3H), 1.59 (s, 3H), 1.55-1.47 (m, 1H), 1.36-1.32 (m, 1H), 1.30 (s, 4H).

### Step 8: Synthesis of Intermediate 1-14

To a mixture of intermediate 1-12 (90 mg, 102.45 μmol, 1 eq, HCl salt) in DCM (1 mL) was added TEA (20.73 mg, 204.90 μmol, 28.52 μL, 2 eq) and intermediate 1-13 (17.36 mg, 153.68 μmol, 12.24 μL, 1.5 eq) at 25° C. The mixture was stirred at 25° C. for 0.5 hr. The reaction mixture was diluted with H₂O (1 mL) and extracted with DCM (1 mL×3), the combined organic layers were dried over Na₂SO₄, filtered and concentrated to give the crude product. The crude product was used for the next step directly. Intermediate 1-14 (90 mg, crude) was obtained as brown gum. LCMS (Method D): Retention time: 0.434 min, (M+1)=918.3.

### Step 9: Synthesis of I-413

To a mixture of intermediate 1-14 (90 mg, 97.99 μmol, 1 eq) in ACN (1 mL) was added intermediate 1-15 (17.81 mg, 146.98 μmol, 18.44 μL, 1.5 eq), KI (32.53 mg, 195.97 μmol, 2 eq) and DIEA (25.33 mg, 195.97 μmol, 34.13 μL, 2 eq) at 25° C. The mixture was stirred at 40° C. for 1 h. The reaction mixture was diluted with H₂O (1 mL) and extracted with DCM (1 mL×3), the combined organic layers were dried over Na₂SO₄, filtered and concentrated to give a residue. The residue was purified by reversed-phase HPLC (0.1% FA condition) and lyophilized to give the product. I-413 (29.66 mg, 27.32 μmol, 27.88% yield, 96.635% purity, FA salt) was obtained as a white solid. LCMS (Method D): Retention time: 0.400 min, (M+1)=1003.5. SFC: Retention time: 2.438 min, 2.807 min. ¹H NMR (400 MHz, METHANOL-d4) δ=8.66-8.55 (m, 2H), 8.43 (s, 1H), 8.40-8.34 (m, 1H), 8.00-7.91 (m, 1H), 7.91-7.78 (m, 2H), 7.57-7.46 (m, 3H), 7.43 (t, J=7.6 Hz, 1H), 7.40-7.34 (m, 1H), 7.31 (d, J=7.6 Hz, 1H), 7.28 (s, 1H), 7.22 (d, J=4.8 Hz, 2H), 7.16 (t, J=8.8 Hz, 1H), 7.08 (s, 1H), 4.39 (s, 2H), 3.86 (s, 2H), 3.79 (d, J=3.6 Hz, 1H), 3.77-3.72 (m, 1H), 3.68 (s, 2H), 3.58-3.49 (m, 3H), 3.49-3.43 (m, 3H), 3.39 (s, 2H), 3.27-3.11 (m, 2H), 2.79-2.71 (m, 3H), 2.71-2.60 (m, 6H), 2.44-2.28 (m, 6H), 2.18-2.07 (m, 1H), 1.95-1.85 (m, 1H), 1.73-1.61 (m, 4H), 1.57 (s, 3H), 1.55-1.48 (m, 1H), 1.40-1.34 (m, 1H), 1.29 (t, J=7.6 Hz, 3H).

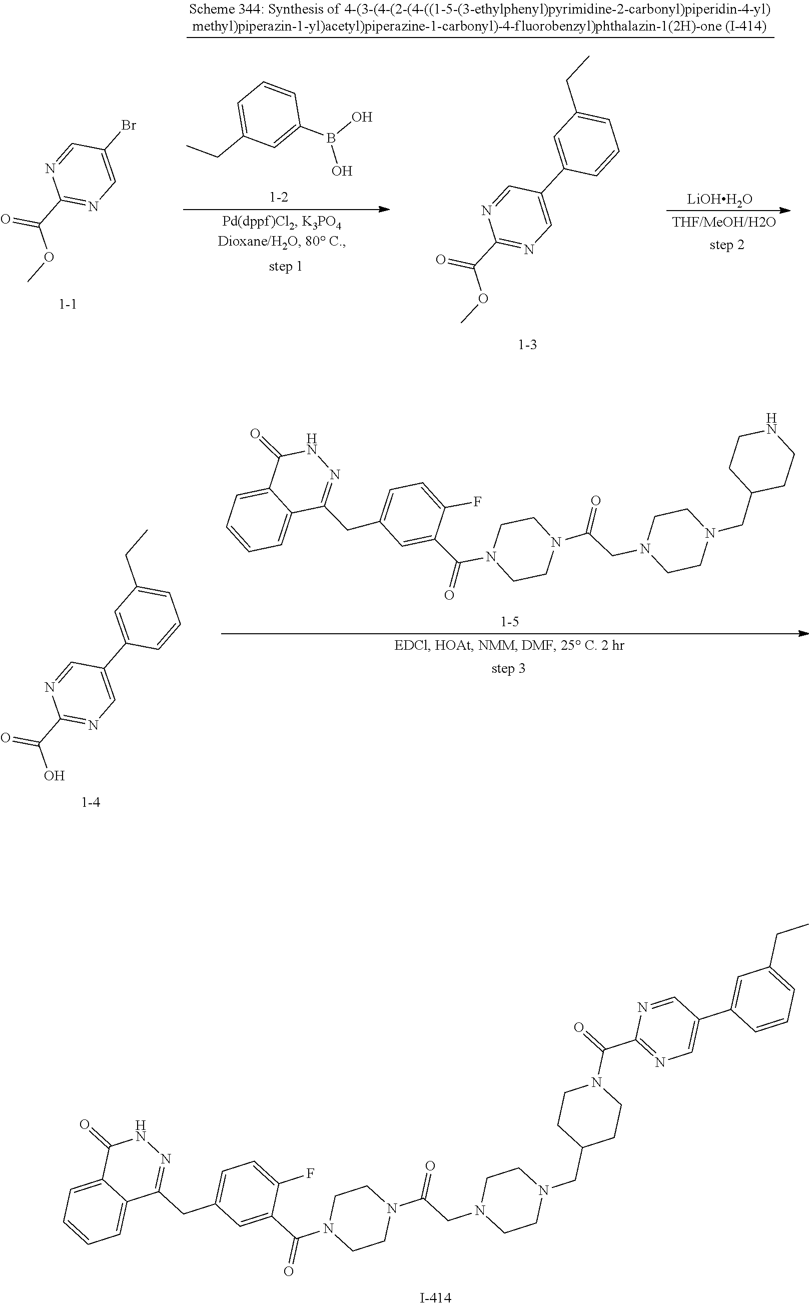

### Step 1: Synthesis of Intermediate 1-3

To a solution of Intermediate 1-1 (200 mg, 921.57 μmol, 1 eq) and Intermediate 1-2 (165.86 mg, 1.11 mmol, 1.2 eq) in dioxane (2 mL) and H₂O (0.4 mL) was added K₃PO₄ (586.86 mg, 2.76 mmol, 3 eq) and ditert-butyl(cyclopentyl)phosphane;dichloropalladium;iron (60.06 mg, 92.16 μmol, 0.1 eq). The mixture was stirred at 80° C. for 1 hr. The reaction mixture was filtered and the filtrate was concentrated. The residue was purified by flash silica gel chromatography (ISCO; 30 g SepaFlash Silica Flash Column, Eluent of 0˜100% Methanol/Dichloromethane Eether gradient @60 mL/min) and the eluent was concentrated to give product. Intermediate 1-3 (200 mg, 774.32 μmol, 84.02% yield, 93.798% purity) was obtained as a white solid. LCMS (Method D): Retention time: 0.332 min, (M+H)=243.1.

### Step 2: Synthesis of Intermediate 1-4

To a solution of Intermediate 1-3 (200 mg, 825.52 μmol, 1 eq) in THF (1 mL) and MeOH (1 mL) and H₂O (1 mL) was added LiOH·H₂O (69.28 mg, 1.65 mmol, 2 eq). The mixture was stirred at 25° C. for 1 hr. To the reaction mixture was added H₂O (5 mL) and then it was extracted with DCM (10 mL*3), the combined organic phase was washed, dried by Na₂SO₄, filtered and concentrated. The crude was used in the next step. Intermediate 1-4 (50 mg, 219.06 μmol, 26.54% yield) was obtained as a white solid. LCMS (Method D): Retention time: 0.306 min, (M+H)=229.1.

### Step 3: Synthesis of I-414

To a solution of Intermediate 1-4 (50 mg, 219.06 μmol, 1 eq) and Intermediate 1-5 (137.17 mg, 219.06 μmol, 1 eq, HCl salt) in DMF (0.5 mL) was added EDCI (83.99 mg, 438.12 μmol, 2 eq) and HOAt (29.82 mg, 219.06 μmol, 30.64 μL, 1 eq) and NMM (110.79 mg, 1.10 mmol, 120.42 μL, 5 eq). The mixture was stirred at 25° C. for 1 hr. The reaction mixture was diluted with MeOH (2 mL). The residue was purified by prep-HPLC (column: Phenomenex luna C18 150*25 mm*10 um; mobile phase: [water (FA)-ACN]; gradient: 15%-45% B over 10 min) and the eluent was concentrated to remove ACN and lyophilized to give product. I-414 (18.6 mg, 21.89 μmol, 9.99% yield, 99.537% purity, FA salt) was obtained as a white solid. LCMS (Method D): Retention time: 0.374 min, (M+H)=800.4. ¹H NMR (400 MHz, METHANOL-d4) δ=9.13 (s, 2H), 8.46 (s, 1H), 8.38 (d, J=7.6 Hz, 1H), 7.95 (d, J=7.2 Hz, 1H), 7.91-7.81 (m, 2H), 7.63-7.54 (m, 2H), 7.53-7.44 (m, 2H), 7.42-7.33 (m, 2H), 7.17 (t, J=9.2 Hz, 1H), 4.67 (d, J=13.6 Hz, 1H), 4.39 (s, 2H), 3.84-3.63 (m, 4H), 3.61-3.49 (m, 3H), 3.37 (s, 3H), 3.30-3.27 (m, 2H), 3.20-3.12 (m, 1H), 2.95 (t, J=12.8 Hz, 1H), 2.79-2.73 (m, 2H), 2.72-2.51 (m, 7H), 2.40-2.34 (m, 2H), 2.03-1.87 (m, 2H), 1.86-1.68 (m, 1H), 1.34-1.26 (m, 5H).

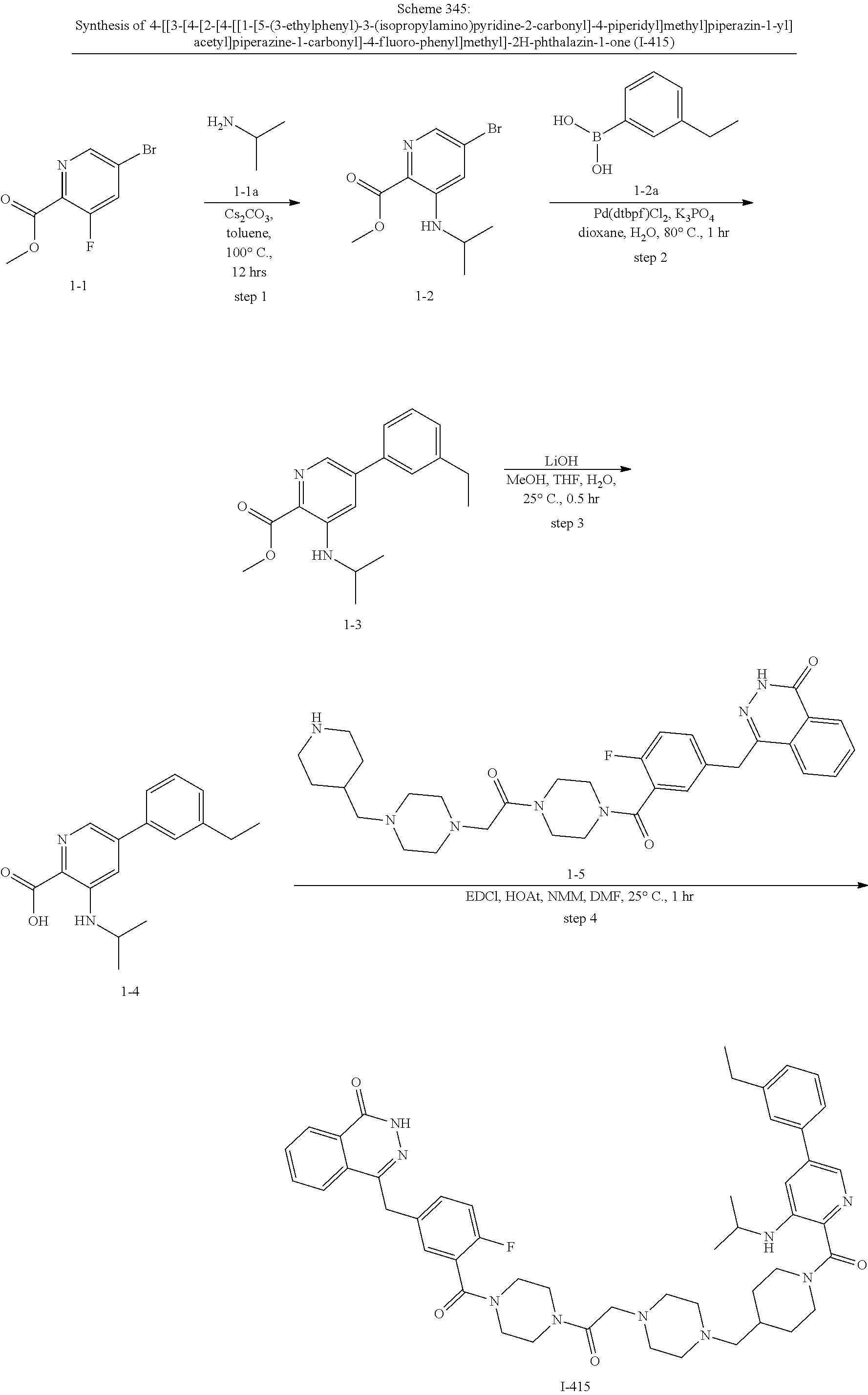

### Step 1: Synthesis of Intermediate 1-2.

To a solution of Intermediate 1-1 (200.00 mg, 854.62 μmol, 1 eq) in DMF (2 mL) was added intermediate 1-1a (50.52 mg, 854.62 μmol, 73.43 μL, 1 eq) and Cs₂CO₃ (835.35 mg, 2.56 mmol, 3 eq). The mixture was stirred at 100° C. for 12 hr. The reaction mixture was diluted with H₂O (2 mL) and extracted with EA (3 mL*2). The combined organic layers were washed with brine (2 mL*1), dried over Na₂SO₄, filtered and concentrated under reduced pressure to give a residue. The residue was purified by flash silica gel chromatography (ISCO; 20 g SepaFlash Silica Flash Column, Eluent of 0˜35% Ethyl acetate/Petroleum ether gradient @60 mL/min). The eluent was concentrated under reduced pressure to give Intermediate 1-2 (150 mg, crude) as yellow liquid. LCMS (Method D): Retention time: 0.467 min, (M+H)=272.7. ¹H NMR (400 MHz, METHANOL-d4) δ=7.87-7.79 (m, 1H), 7.53-7.47 (m, 1H), 3.90 (s, 3H), 3.83-3.70 (m, 1H), 1.29-1.26 (m, 6H).

### Step 2: Synthesis of Intermediate 1-3.

To a solution of Intermediate 1-2 (150 mg, 549.20 μmol, 1 eq) in H₂O (0.3 mL) and 1,4-dioxane (1.5 mL) was added intermediate 1-2a (98.84 mg, 659.04 μmol, 1.2 eq), K₃PO₄ (349.73 mg, 1.65 mmol, 3 eq) and Pd(dtbpf)Cl₂ (35.79 mg, 54.92 μmol, 0.1 eq). The mixture was stirred at 80° C. for 1 hr under N₂. The reaction mixture was diluted with H₂O (3 mL) and extracted with EA (3 mL*2). The combined organic layers were washed with brine (2 mL*1), dried over Na₂SO₄, filtered and concentrated under reduced pressure to give a residue. The residue was purified by flash silica gel chromatography (ISCO; 12 g SepaFlash Silica Flash Column, Eluent of 0˜25% Ethyl acetate/Petroleum ether gradient @60 mL/min). The eluent was concentrated under reduced pressure to give Intermediate 1-3 (150 mg, crude, HCl salt) as yellow oil. LCMS (Method D): Retention time: 0.450 min, (M+H)=299.0.

### Step 3: Synthesis of Intermediate 1-4.

To a solution of Intermediate 1-3 (130 mg, 435.69 μmol, 1 eq) in MeOH (0.5 mL), THF (0.5 mL) and H₂O (0.5 mL) was added LiOH·H₂O (54.85 mg, 1.31 mmol, 3 eq). The mixture was stirred at 25° C. for 0.5 hr. The mixture was diluted with H₂O (2 mL) and HCl (1M) was added to adjust pH to 4. The mixture was extracted with EA (2 mL*2). The combined organic layers were dried over Na₂SO₄, filtered and concentrated under reduced pressure to give a residue to give the crude Intermediate 1-4 (120 mg, 397.65 μmol, 91.27% yield, 94.227% purity) as a yellow gum that was used in the next step without further purification. LCMS (Method D): Retention time: 0.408 min, (M+H)=285.1.

### Step 4: Synthesis of I-414

To a solution of Intermediate 1-4 (28.93 mg, 101.75 μmol, 1 eq) in DMF (1 mL) was added Intermediate 1-5 (60 mg, 101.75 μmol, 1 eq), EDCI (58.51 mg, 305.24 μmol, 3 eq), HOAt (13.85 mg, 101.75 μmol, 14.23 μL, 1 eq) and NMM (51.46 mg, 508.73 μmol, 55.93 μL, 5 eq). The mixture was stirred at 25° C. for 1 hr. The reaction mixture was diluted with H₂O (2 mL) and extracted with EA (2 mL*2). The combined organic layers were washed with brine (2 mL*1), dried over Na₂SO₄, filtered and concentrated under reduced pressure to give a residue. The crude product was purified by prep-HPLC (column: Phenomenex luna C18 150*25 mm*10 um; mobile phase: [water (FA)-ACN]; gradient: 20%-500% B over 8 min). The eluent was concentrated under reduced pressure to remove ACN and then lyophilized to give I-414 (10.87 mg, 11.99 μmol, 11.78% yield, 99.473% purity, FA salt) was obtained as a white solid. LCMS (Method D): Retention time: 0.401 min, (M+H)=856.4. ¹H NMR (400 MHz, METHANOL-d₄) δ=8.45 (s, 1H), 8.39-8.31 (m, 1H), 8.06-8.00 (m, 1H), 7.98-7.91 (m, 1H), 7.90-7.79 (m, 2H), 7.52-7.34 (m, 5H), 7.33-7.31 (m, 1H), 7.30-7.25 (m, 1H), 7.20-7.12 (m, 1H), 4.74-4.55 (m, 1H), 4.38 (s, 2H), 3.85-3.63 (m, 6H), 3.57-3.47 (m, 2H), 3.46-3.31 (m, 4H), 3.21-3.06 (m, 1H), 2.95-2.85 (m, 1H), 2.84-2.57 (m, 10H), 2.54-2.39 (m, 2H), 2.05-1.85 (m, 2H), 1.83-1.62 (m, 1H), 1.32-1.19 (m, 11H). ¹⁹FNMR (376 MHz, MeOD-d₆) δ=−120.712.

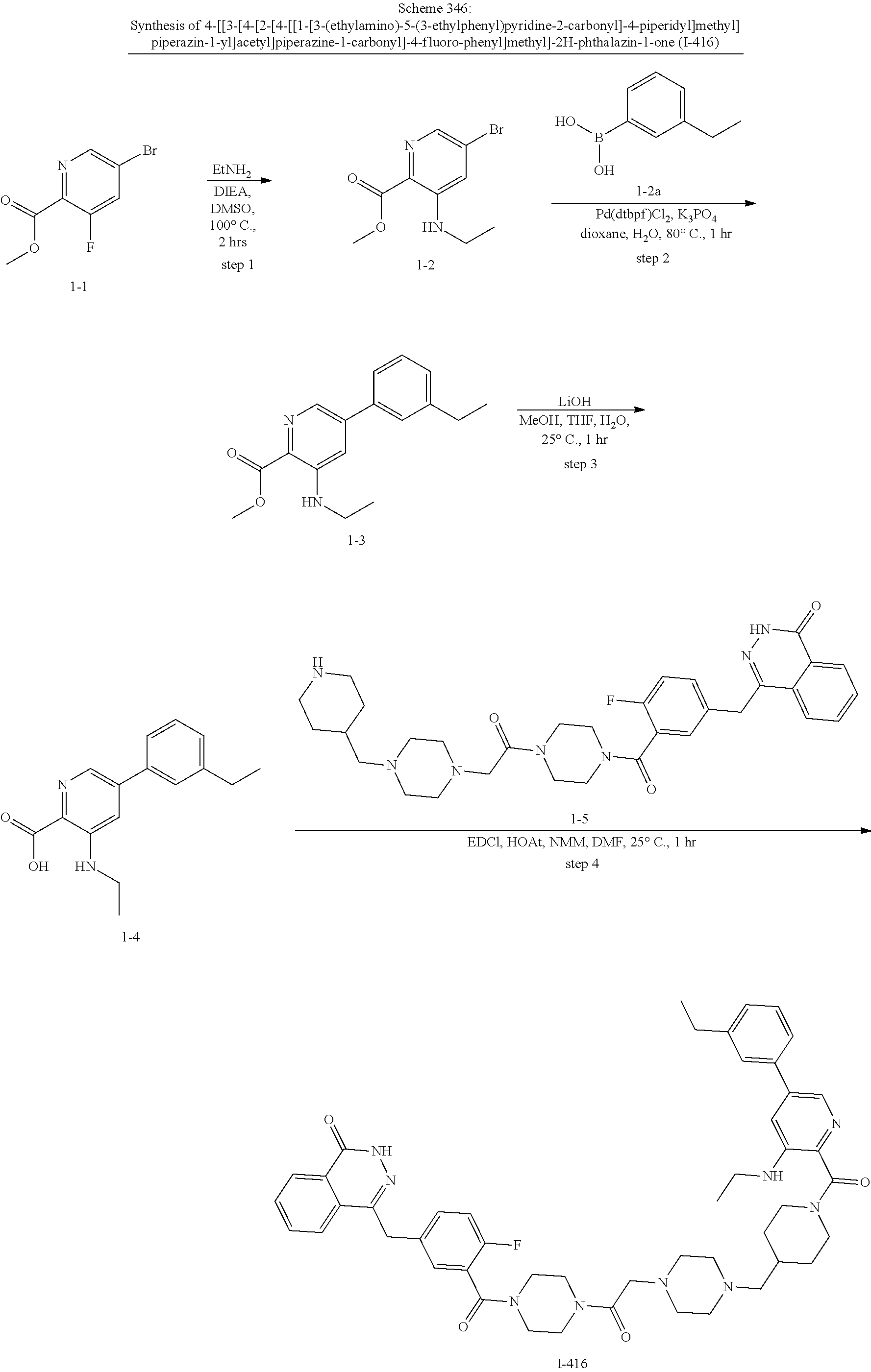

### Step 1: Synthesis of Intermediate 1-2.

To a solution of Intermediate 1-1 (500 mg, 2.14 mmol, 1 eq) in DMSO (5 mL) was added EtNH₂ (2 M, 1.60 mL, 1.5 eq) and DIEA (828.40 mg, 6.41 mmol, 1.12 mL, 3 eq). The mixture was stirred at 100° C. for 2 hrs. The reaction mixture was diluted with H₂O (5 mL) and extracted with EA (5 mL*2). The combined organic layers were washed with brine (3 mL*1), dried over Na₂SO₄, filtered and concentrated under reduced pressure to give a residue. The residue was purified by flash silica gel chromatography (ISCO; 40 g SepaFlash Silica Flash Column, Eluent of 0˜30% Ethyl acetate/Petroleum ether gradient @60 mL/min). The eluent was concentrated under reduced pressure to give Intermediate 1-2 (660 mg, crude) as yellow oil. LCMS (Method D): Retention time: 0.389 min, (M+H)=258.9.

### Step 2: Synthesis of Intermediate 1-3.

To a solution of Intermediate 1-2 (400 mg, 1.54 mmol, 1 eq) in H₂O (1 mL) and 1,4-dioxane (5 mL) was added intermediate 1-2a (277.85 mg, 1.85 mmol, 1.2 eq), K₃PO₄ (983.10 mg, 4.63 mmol, 3 eq) and Pd(dtpbf)Cl₂ (100.62 mg, 154.38 μmol, 0.1 eq). The mixture was stirred at 80° C. for 1 hr under N₂. The reaction mixture was diluted with H₂O (2 mL) and extracted with EA (2 mL*2). The combined organic layers were washed with brine (2 mL*1), dried over Na₂SO₄, filtered and concentrated under reduced pressure to give a residue. The residue was purified by flash silica gel chromatography (ISCO; 20 g SepaFlash Silica Flash Column, Eluent of 0˜30% Ethyl acetate/Petroleum ether gradient @60 mL/min). The eluent was concentrated under reduced pressure to give Intermediate 1-3 (360 mg, 1.18 mmol, 76.59% yield, 93.4% purity) as a yellow oil. LCMS (Method D): Retention time: 0.429 min, (M+H)=285.0. ¹H NMR (400 MHz, METHANOL-d₄) δ=8.14-7.95 (m, 1H), 7.55-7.18 (m, 5H), 3.92 (s, 3H), 3.36-3.31 (m, 2H), 2.76-2.68 (m, 2H), 1.36-1.25 (m, 6H).

### Step 3: Synthesis of Intermediate 1-4.

To a solution of Intermediate 1-3 (200 mg, 703.35 μmol, 1 eq) in MeOH (0.5 mL), THF (0.5 mL) and H₂O (0.5 mL) was added LiOH·H₂O (88.55 mg, 2.11 mmol, 3 eq). The mixture was stirred at 25° C. for 1 hr. The mixture was diluted with H₂O (1 mL) and HCl (1M) was added to adjust pH to 4. The mixture was extracted with EA (1 mL*2). The combined organic layers were dried over Na₂SO₄, filtered and concentrated under reduced pressure to give crude Intermediate 1-4 (230 mg, crude) as a yellow gum, which was used in the next step without further purification. LCMS (Method D): Retention time: 0.385 min, (M+H)=271.0. ¹H NMR (400 MHz, METHANOL-d₄) δ=8.06-7.89 (m, 1H), 7.65-7.25 (m, 5H), 3.43-3.33 (m, 2H), 2.78-2.66 (m, 2H), 1.38-1.22 (m, 6H).

### Step 4: Synthesis of I-416

To a solution of Intermediate 1-4 (36.67 mg, 135.66 μmol, 1 eq) in DMF (1 mL) was added Intermediate 1-5 (80 mg, 135.66 μmol, 1 eq), EDCI (78.02 mg, 406.99 μmol, 3 eq), HOAt (18.47 mg, 135.66 μmol, 18.98 μL, 1 eq) and NMM (68.61 mg, 678.31 μmol, 74.57 μL, 5 eq). The mixture was stirred at 25° C. for 1 hr. The reaction mixture was diluted with H₂O (1 mL) and extracted with EA (2 mL*2). The combined organic layers were washed with brine (2 mL*1), dried over Na₂SO₄, filtered and concentrated under reduced pressure to give a residue. The crude product was purified by prep-HPLC (column: Phenomenex luna C18 150*25 mm*10 um; mobile phase: [water (FA)-ACN]; gradient: 18%-48% B over 10 min). The eluent was concentrated under reduced pressure to remove ACN and then lyophilized to give I-416 (16.21 mg, 19.07 μmol, 14.06% yield, 99.058% purity) as a yellow solid. LCMS (Method D): Retention time: 0.391 min, (M+H)=842.5. ¹H NMR (400 MHz, METHANOL-d₄) δ=8.55-8.47 (m, 1H), 8.40-8.33 (m, 1H), 8.06-8.00 (m, 1H), 7.98-7.92 (m, 1H), 7.90-7.79 (m, 2H), 7.49-7.35 (m, 5H), 7.31-7.24 (m, 2H), 7.20-7.12 (m, 1H), 4.70-4.60 (m, 1H), 4.38 (s, 2H), 3.85-3.61 (m, 6H), 3.57-3.48 (m, 2H), 3.36-3.33 (m, 1H), 3.29-3.21 (m, 4H), 3.17-3.07 (m, 1H), 2.95-2.85 (m, 1H), 2.77-2.70 (m, 2H), 2.63-2.43 (m, 8H), 2.31-2.23 (m, 2H), 1.95-1.85 (m, 2H), 1.80-1.69 (m, 1H), 1.32-1.24 (m, 8H). ¹⁹FNMR (376 MHz, MeOD-d₆) δ=−120.802.

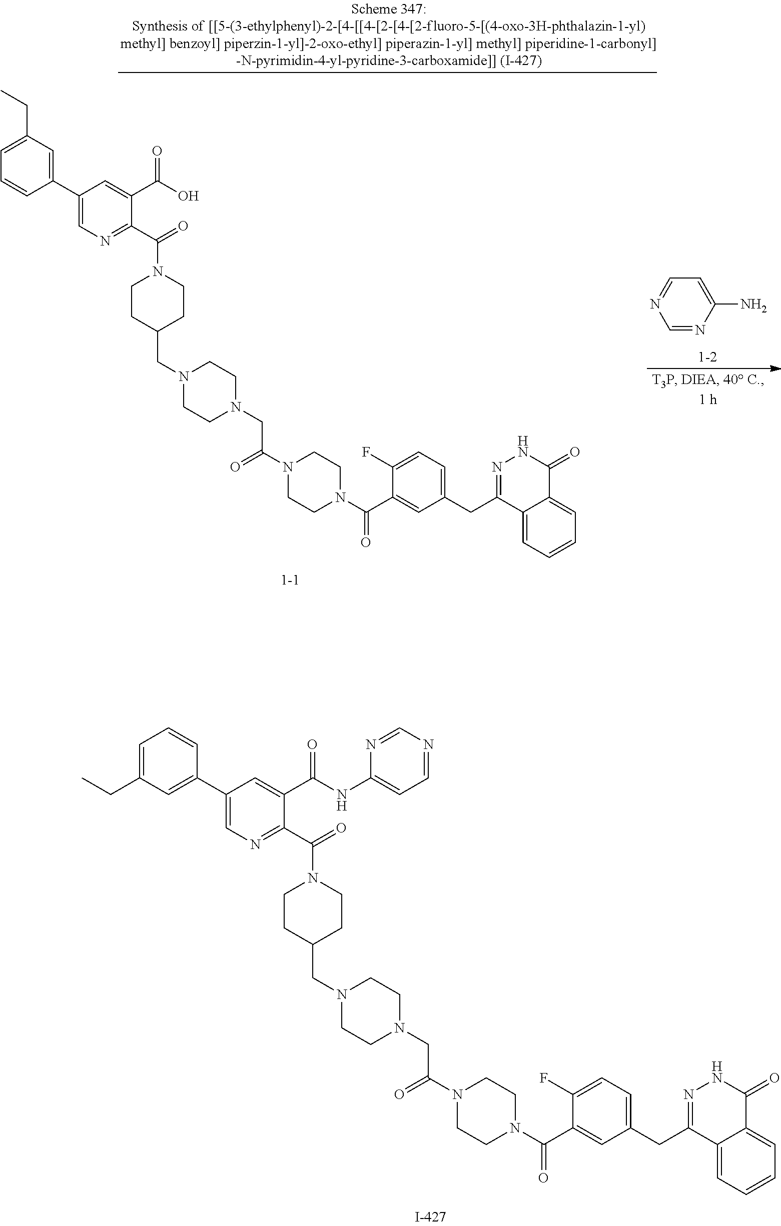 [SMILES: CCc1cccc(-c2cnc(C(=O)N3CCC(CN4CCN(CC(=O)N5CCN(C(=O)c6cc(Cc7n[nH]c(=O)c8ccccc78)ccc6F)CC5)CC4)CC3)c(C(=O)O)c2)c1 ; Nc1ccncn1 ; CCc1cccc(-c2cnc(C(=O)N3CCC(CN4CCN(CC(=O)N5CCN(C(=O)c6cc(Cc7n[nH]c(=O)c8ccccc78)ccc6F)CC5)CC4)CC3)c(C(=O)Nc3ccncn3)c2)c1]

To a solution of intermediate 1-1 (100 mg, 118.63 μmol, 1 eq) and intermediate 1-2 (11.28 mg, 118.63 μmol, 1 eq) in DMF (1 mL) was added T₃P (256.43 mg, 355.89 μmol, 50% purity, 3 eq) and DIEA (46.00 mg, 355.89 μmol, 61.99 μL, 3 eq). The mixture was stirred at 40° C. for 1 hr. The reaction mixture was poured into H₂O (2 mL) and extracted with EA (2 mL*3). The combined organic layers were dried over Na₂SO₄, filtered and concentrated under reduced pressure to give a residue. The residue was purified by prep-HPLC (column: CD02-Waters Xbidge BEH C18 150*25*10 um; mobile phase: [water (NH₄HCO₃)-ACN]; gradient: 27%-57% B over 10 min) and concentrated under reduced pressure to remove ACN and lyophilized to give product. I-427 (9.09 mg, 9.85 μmol, 8.30% yield, 99.677% purity) was obtained as a yellow solid. LCMS (Method H): Rt=0.602 min, (M+H)=920.5. ¹H NMR (400 MHz, METHANOL-d4) δ=8.97 (d, J=2.0 Hz, 1H), 8.89 (s, 1H), 8.68 (d, J=5.6 Hz, 1H), 8.51 (d, J=2.0 Hz, 1H), 8.37 (d, J=8.0 Hz, 1H), 8.29 (d, J=6.0 Hz, 1H), 7.97-7.91 (m, 1H), 7.90-7.80 (m, 2H), 7.67 (s, 1H), 7.62 (d, J=7.6 Hz, 1H), 7.52-7.48 (m, 1H), 7.47-7.43 (m, 1H), 7.41-7.30 (m, 2H), 7.16 (t, J=8.8 Hz, 1H), 4.80 (s, 2H), 4.68-4.51 (m, 2H), 4.39 (s, 2H), 3.82-3.62 (m, 4H), 3.61-3.48 (m, 3H), 3.27 (s, 1H), 3.23-3.12 (m, 2H), 2.92 (t, J=13.2 Hz, 1H), 2.77 (q, J=7.6 Hz, 2H), 2.52 (s, 7H), 2.31-2.17 (m, 2H), 1.96-1.83 (m, 2H), 1.73 (d, J=12.4 Hz, 1H), 1.53-1.33 (m, 2H), 1.33-1.28 (m, 3H).

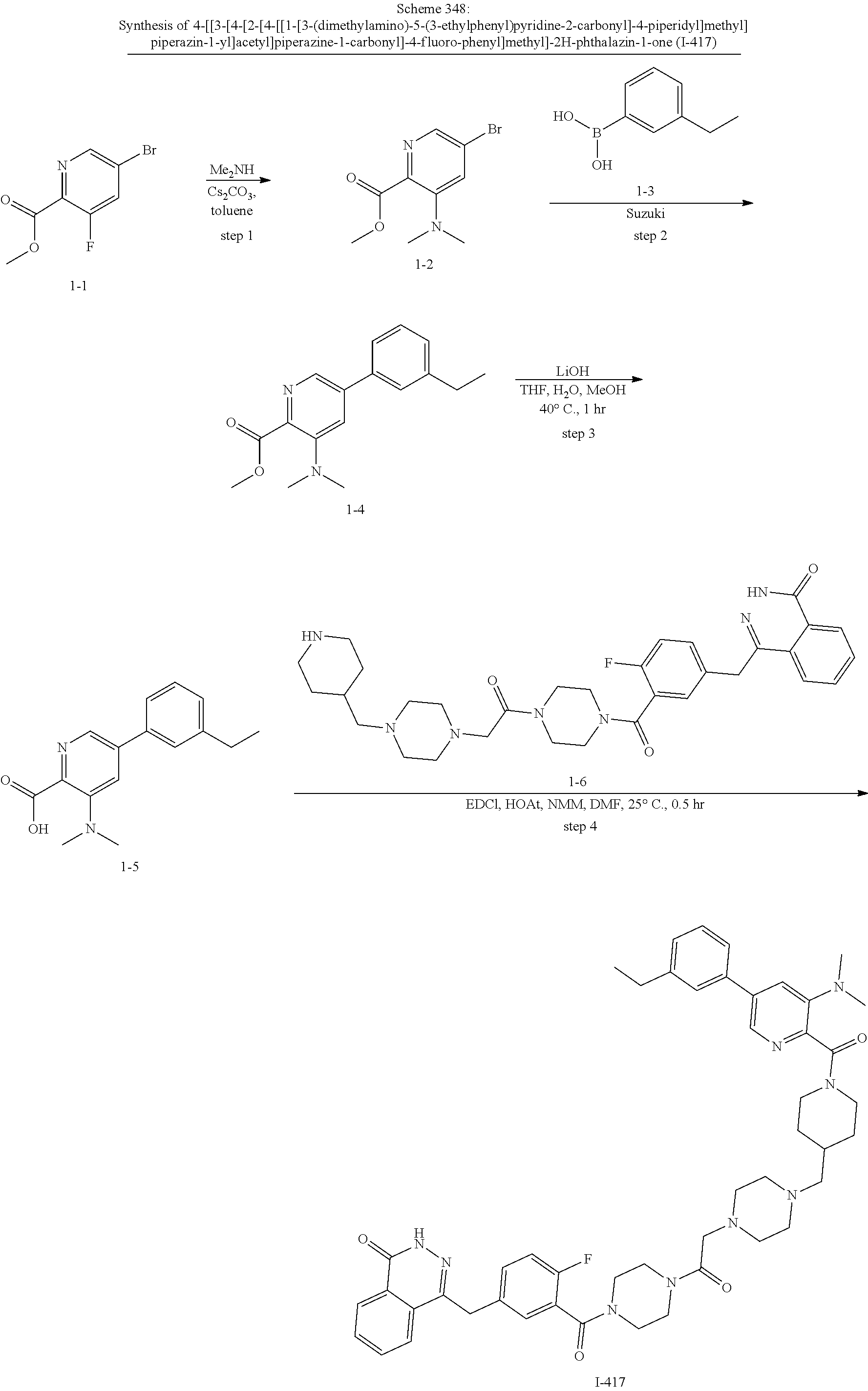

### Step 1: Synthesis of Intermediate 1-2

To a solution of Intermediate 1-1 (100 mg, 427.31 μmol, 1 eq) in DMF (1 mL) was added Me₂NH (48.16 mg, 427.31 μmol, 54.11 μL, 1 eq) and Cs₂CO₃ (417.68 mg, 1.28 mmol, 3 eq). The mixture was stirred at 100° C. for 1 hr. The reaction mixture was diluted with water (21 mL) and extracted with ethyl acetate (7 mL×3). The combined organic layers were washed with brine (7 mL×3) and dried over sodium sulfate, filtered and concentrated under reduced pressure to give Intermediate 1-2 (100 mg, 385.95 μmol, 90.32% yield, 100% purity) as a yellow oil. LCMS (Method D): Rt: 0.320 min, (M+H)=258.9.

### Step 2: Synthesis of Intermediate 1-4

To a solution of Intermediate 1-2 (100 mg, 385.95 μmol, 1 eq) and Intermediate 1-3 (57.89 mg, 385.95 μmol, 1 eq) in H₂O (0.3 mL) and dioxane (1 mL) was added cyclopentyl (diphenyl) phosphane; dichloropalladium; iron (28.24 mg, 38.60 μmol, 0.1 eq) and dipotassium; carbonate (160.02 mg, 1.16 mmol, 3 eq). The suspension was stirred at 90° C. for 1 hr under N₂. The reaction mixture was diluted with water (18 mL) and extracted with ethyl acetate (6 mL×3). The combined organic layers were washed with brine (6 mL×3) and dried over sodium sulfate, filtered and concentrated under reduced pressure to give a residue. The residue was purified by flash silica gel chromatography (ISCO; 12 g SepaFlash Silica Flash Column, Eluent of 0˜10% Petroleum ether:Ethyl acetate gradient @30 mL/min). The eluent was concentrated under reduced pressure to give Intermediate 1-4 (80 mg, 275.43 μmol, 71.36% yield, 97.9% purity) as yellow oil. LCMS (Method D): Rt: 0.400 min, (M+H)=285.0.

### Step 3: Synthesis of Intermediate 1-5

To a solution of Intermediate 1-4 (100 mg, 351.68 μmol, 1 eq) in THF (0.4 mL), H₂O (0.1 mL), and MeOH (0.4 mL) was added LiOH·H₂O (14.76 mg, 351.68 μmol, 1 eq). The mixture was stirred at 50° C. for 1 hr. The pH of the reaction mixture was adjusted to 6.5 with citric acid monohydrate. The reaction mixture was diluted with water (15 mL) and extracted with ethyl acetate (5 mL×3). The combined organic layers were washed with brine (5 mL×3) and dried over sodium sulfate, filtered and concentrated under reduced pressure to give Intermediate 1-5 (80 mg, 273.15 μmol, 77.67% yield, 92.3% purity) as yellow oil. LCMS (Method D): Rt: 0.315 min, (M+H)=271.0.

### Step 4: Synthesis of I-417

To a solution of Intermediate 1-6 (185.31 mg, 295.94 μmol, 1 eq, HCl salt) in DCM (2 mL) was added EDCI (170.20 mg, 887.82 μmol, 3 eq), HOAt (40.28 mg, 295.94 μmol, 41.40 μL, 1 eq), and NMM (149.67 mg, 1.48 mmol, 162.68 μL, 5 eq) at 25° C. for 0.5 hr. Then Intermediate 1-5 (80 mg, 295.94 μmol, 1 eq) was added into the mixture and it was stirred at 25° C. for 0.5 hr. The reaction mixture was diluted with water (9 mL) and extracted with DCM (3 mL×3). The combined organic layers were washed with brine (3 mL×3) and dried over sodium sulfate, filtered and concentrated under reduced pressure to give a residue. The crude product was purified by reversed-phase HPLC (column: CD01-Phenomenex luna C18 150*25*10 um; mobile phase: [water (FA)-ACN]; gradient: 16%-46% B over 5 min) and the eluent was concentrated under reduced pressure to remove ACN and then lyophilized to give I-417 (35.45 mg, 37.96 μmol, 12.83% yield, 95.08% purity, FA salt) as a white solid. LCMS (Method D): Rt: 0.380 min, (M+H)=842.3. ¹H NMR (400 MHz, METHANOL-d4) δ=8.40 (s, 1H), 8.39-8.35 (m, 1H), 8.26 (d, J=1.6 Hz, 1H), 8.00-7.92 (m, 1H), 7.91-7.80 (m, 2H), 7.58 (d, J=1.6 Hz, 1H), 7.53-7.34 (m, 5H), 7.29 (d, J=7.6 Hz, 1H), 7.19-7.17 (m, 1H), 4.76-4.65 (m, 1H), 4.39 (s, 2H), 3.83-3.72 (m, 2H), 3.68 (s, 2H), 3.58-3.45 (m, 4H), 3.41 (s, 1H), 3.38-3.33 (m, 1H), 3.27-2.97 (m, 2H), 2.95 (s, 6H), 2.93-2.68 (m, 11H), 2.63-2.51 (m, 2H), 2.11-1.91 (m, 2H), 1.76 (d, J=12.8 Hz, 1H), 1.31-1.27 (m, 5H). ¹⁹F NMR (376 MHz, METHANOL-d4) δ=−120.779.

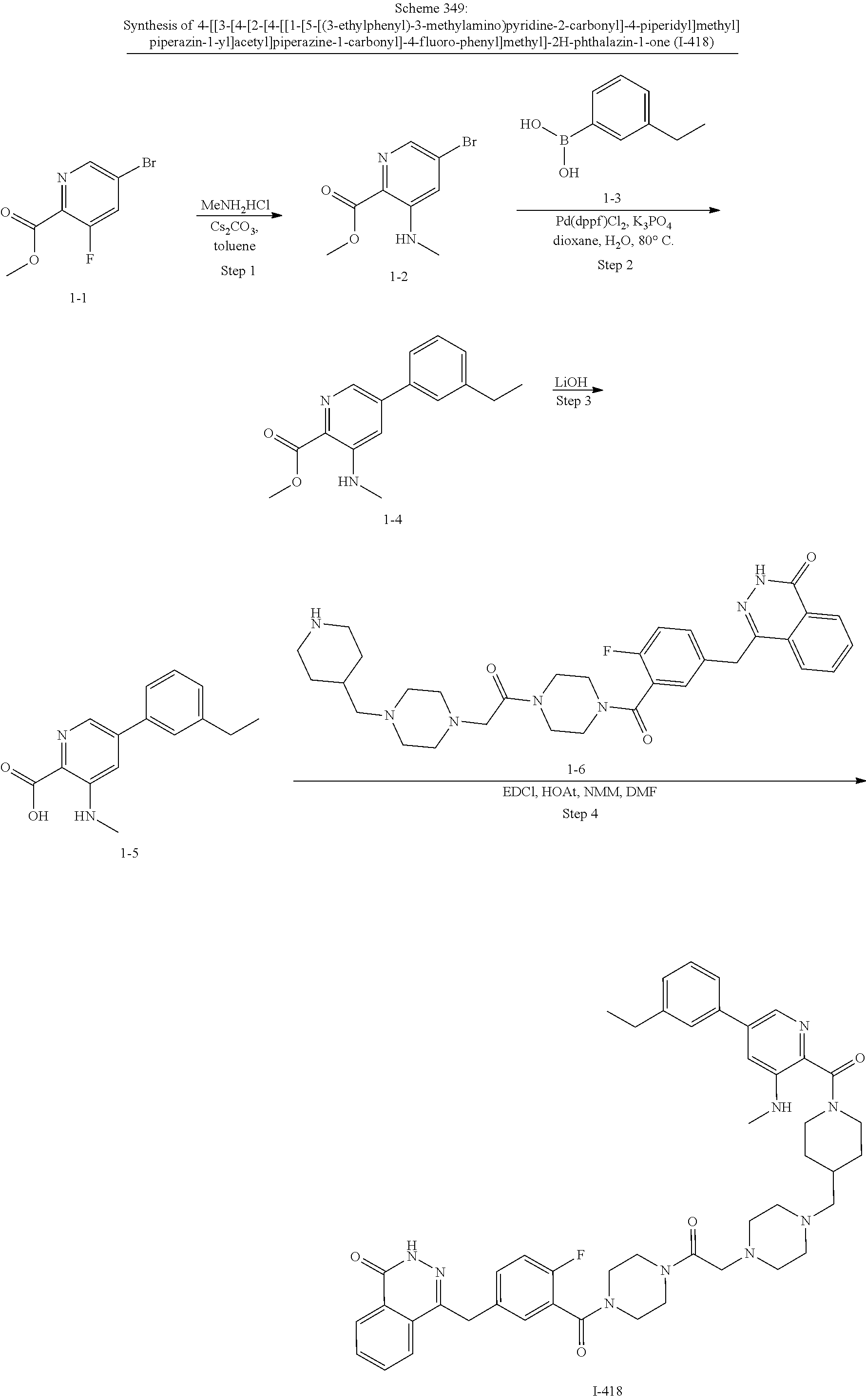

### Step 1: Synthesis of Intermediate 1-2.

To a mixture of Intermediate 1-1 (400 mg, 1.71 mmol, 1 eq) and methanamine; hydrochloride (461.62 mg, 6.84 mmol, 4 eq) in toluene (5 mL) was added Cs₂CO₃ (2.23 g, 6.84 mmol, 4 eq), then the mixture was stirred at 105° C. for 16 hours under N₂. The reaction mixture was concentrated under reduced pressure to give a residue. The residue was purified by flash silica gel chromatography (ISCO; 12 g SepaFlash Silica Flash Column, Eluent of 0˜30% Ethyl acetate/Petroleum ether gradient @80 mL/min). The eluent was concentrated under reduced pressure to give Intermediate 1-2 (200 mg, 709.99 μmol, 41.54% yield, 87% purity) as a white solid. LCMS (Method D): Retention time: 0.344 min, (M+H)=247.0.

### Step 2: Synthesis of Intermediate 1-4.

To a mixture of Intermediate 1-2 (150 mg, 612.06 μmol, 1 eq) and Intermediate 1-3 (137.70 mg, 918.09 μmol, 1.5 eq) in dioxane (2 mL) and H₂O (0.4 mL) was added Pd(dppf)Cl₂ (44.78 mg, 61.21 μmol, 0.1 eq) and K₃PO₄ (389.76 mg, 1.84 mmol, 3 eq), then the mixture was stirred at 80° C. for 1 hour under N₂. The reaction mixture was quenched with water (20 mL) and extracted with EA (5 mL*2). The combined organic layers were concentrated under reduced pressure to give a residue. The residue was purified by flash silica gel chromatography (ISCO; 20 g SepaFlash Silica Flash Column, Eluent of 0-50% Ethylacetate/Petroleum ether gradient @60 mL/min). The eluent was concentrated under reduced pressure to give Intermediate 1-4 (150 mg, 471.65 μmol, 77.06% yield, 85% purity) as a white solid. LCMS (Method D): Retention time: 0.381 min, (M+H)=271.2. ¹H NMR (400 MHz, DMSO-d6) δ=8.16 (d, J=2.0 Hz, 1H), 7.96 (s, 1H), 7.62 (s, 1H), 7.57 (s, 1H), 7.43 (t, J=7.6 Hz, 1H), 7.34 (d, J=2.0 Hz, 1H), 7.26-7.21 (m, 1H), 3.83 (s, 3H), 2.95 (d, J=5.2 Hz, 3H), 2.70 (q, J=7.6 Hz, 2H), 1.24 (t, J=7.6 Hz, 3H).

### Step 3: Synthesis of Intermediate 1-5.

To a mixture of Intermediate 1-4 (120 mg, 443.91 μmol, 1 eq) in THF (2 mL), MeOH (1 mL) and H₂O (0.5 mL) was added LiOH·H₂O (55.88 mg, 1.33 mmol, 3 eq), the mixture was stirred at 20° C. for 0.5 hour. The reaction mixture was concentrated under reduced pressure to remove THF, and then 1 N HCl was added (about 3 mL) to adjust the pH to 3-4. The mixture was extracted with EA (10 mL*3). The combined organic layers were concentrated under reduced pressure to give Intermediate 1-5 (70 mg, 273.12 μmol, 61.53% yield) as a white solid. LCMS (Method D): Retention time: 0.353 min, (M+H)=257.2.

### Step 4: Synthesis of I-418

To a mixture of Intermediate 1-5 (50 mg, 195.08 μmol, 1 eq) and Intermediate 1-6 (115.04 mg, 195.08 μmol, 1 eq) in DMF (2 mL) was added HOAt (26.55 mg, 195.08 μmol, 27.29 μL, 1 eq), EDCI (112.19 mg, 585.25 μmol, 3 eq), NMM (98.66 mg, 975.42 μmol, 107.24 μL, 5 eq), then the mixture was stirred at 20° C. for 1 hour. The reaction mixture was diluted with water (50 mL) and extracted with EA (10 mL*3). The combined organic layers were concentrated under reduced pressure to give a residue. The crude product was purified by reversed-phase HPLC (0.1% FA condition). The eluent was concentrated under reduced pressure to remove MeCN and then lyophilized to give I-418 (19.29 mg, 21.83 μmol, 11.19% yield, 98.9% purity, FA salt) as a yellow solid. LCMS (Method D): Retention time: 0.377 min, (M+H)=828.3. ¹H NMR (400 MHz, METHANOL-d4) δ=8.48-8.43 (m, 1H), 8.37 (d, J=7.6 Hz, 1H), 8.03 (s, 1H), 7.99-7.92 (m, 1H), 7.90-7.82 (m, 2H), 7.53-7.45 (m, 3H), 7.43-7.35 (m, 2H), 7.30-7.25 (m, 2H), 7.19-7.16 (m, 1H), 4.69-4.65 (m, 1H), 4.39 (s, 2H), 3.81-3.66 (m, 5H), 3.52 (br s, 2H), 3.40-3.32 (m, 4H), 3.16-3.09 (m, 1H), 2.93-2.86 (m, 4H), 2.82-2.53 (m, 10H), 2.44-2.35 (m, 2H), 1.98-1.86 (m, 2H), 1.82-1.69 (m, 1H), 1.32-1.24 (m, 5H). ¹⁹F NMR (376 MHz, METHANOL-d4) δ=−120.76.

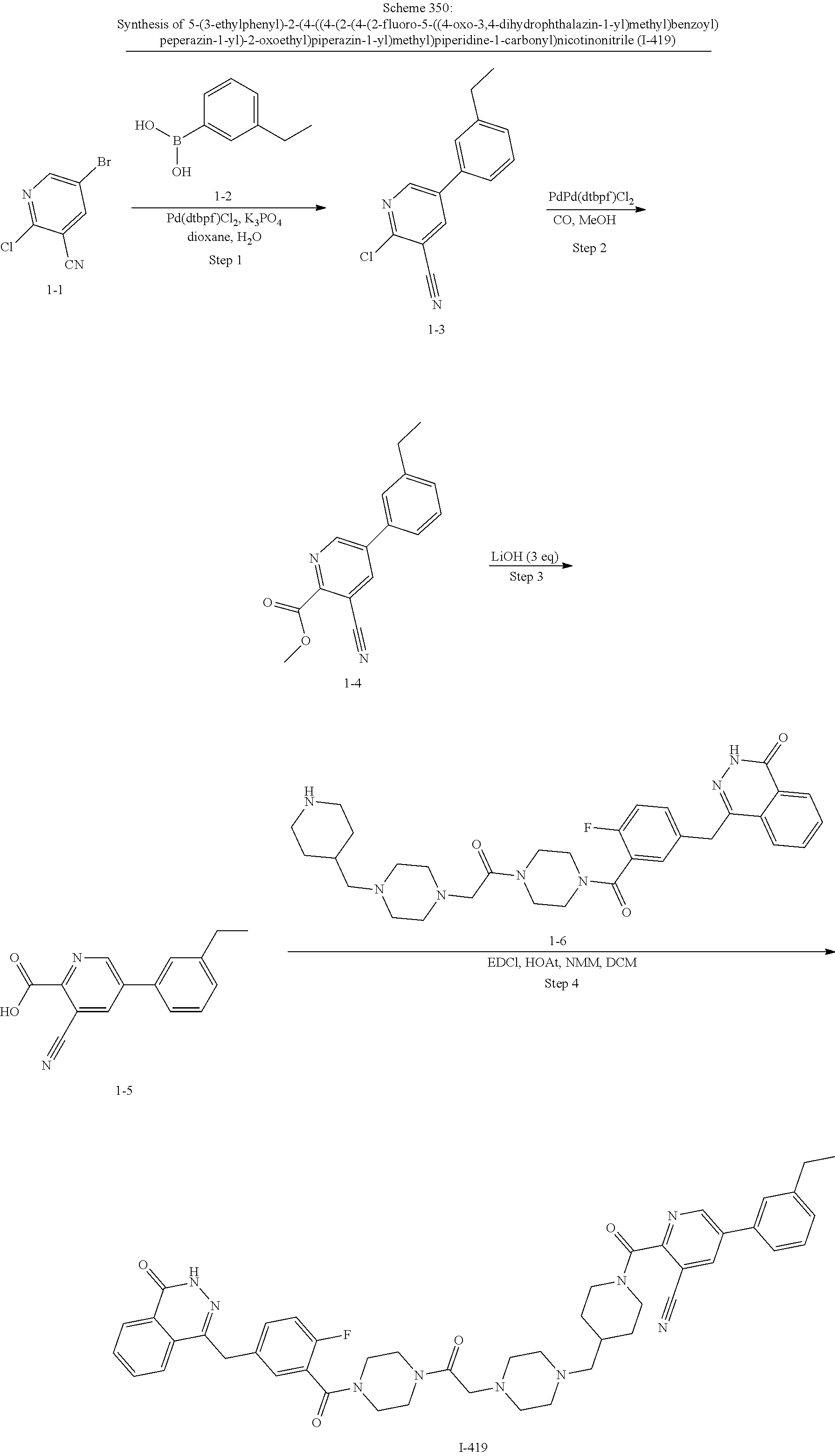

### Step 1: Synthesis of Intermediate 1-3

A mixture of Intermediate 1-1 (1 g, 4.60 mmol, 1 eq), Intermediate 1-2 (758.70 mg, 5.06 mmol, 1.1 eq), K₃PO₄ (2.93 g, 13.80 mmol, 3 eq), and Pd(dppf)Cl₂ (336.49 mg, 459.87 umol, 0.1 eq) in dioxane (10 mL) and H₂O (1 mL) was degassed and purged with N₂ 3 times, and then the mixture was stirred at 80° C. for 0.5 hr under N₂ atmosphere. The reaction mixture was diluted with H₂O (10 mL) and extracted with EA (10 mL*3). The combined organic layers were dried over Na₂SO₄, filtered and concentrated under reduced pressure to give a residue. The residue was purified by flash silica gel chromatography (ISCO; 40 g SepaFlash Silica Flash Column, Eluent of 0˜50% Ethyl acetate/Petroleum ether gradient @120 mL/min, Rf=0.46 (PE:EA=5:1), the eluent was concentrated under reduced pressure to give Intermediate 1-3 (1.1 g, 4.53 mmol, 98.55% yield) as a white solid. LCMS (Method E): Rt=0.483 min, [M+H]=242.9.

### Step 2: Synthesis of Intermediate 1-4

A mixture of Intermediate 1-3 (500 mg, 2.06 mmol, 1 eq), cyclopentyl (diphenyl) phosphane;dichloropalladium;iron (150.74 mg, 206.01 μmol, 0.1 eq) and TEA (625.39 mg, 6.18 mmol, 860.23 μL, 3 eq) in MeOH (5 mL) was degassed and purged with N₂ 3 times, and then purged with CO (2.06 mmol, 50 psi) 3 times and the mixture was stirred at 60° C. for 12 hr under CO (50 psi) atmosphere. The reaction mixture was poured into water (10 mL) and extracted with EA (15 mL*3). The combined organic layers were dried over Na₂SO₄, filtered and concentrated under reduced pressure to give a residue. The residue was purified by flash silica gel chromatography (ISCO; 40 g SepaFlash Silica Flash Column, Eluent of 0-50% Ethyl acetate/Petroleum ether gradient @120 mL/min Rf=0.15 (PE/EA=5:1). The eluent was concentrated under reduced pressure to give Intermediate 1-4 as a white solid. LCMS (Method E): Rt=0.314 min, [M+H]⁺=260.9.

### Step 3: Synthesis of Intermediate 1-5

To a solution of Intermediate 1-4 (200 mg, 751.05 μmol, 1 eq) in DCM (5 mL), MeOH (2.5 mL) and H₂O (2.5 mL) was added LiOH·H₂O (94.55 mg, 2.25 mmol, 3 eq). The mixture was stirred at 25° C. for 2 hr. The reaction mixture was quenched by addition of 1 M HCl to adjust the pH to 7, and then the mixture was extracted with EA (5 mL*3). The organics were dried by Na₂SO₄, filtered and concentrated under reduced pressure to give Intermediate 1-5 (150 mg, 594.61 μmol, 79.17% yield) as a white solid. LCMS (Method E): Rt=0.424 min, [M+H]⁺=527.1.

### Step 4: Synthesis of I-419

To a solution of Intermediate 1-5 (100 mg, 396.40 μmol, 1 eq) in DMF (2 mL) was added HOAt (53.96 mg, 396.40 μmol, 55.45 μL, 1 eq), EDCI (151.98 mg, 792.81 μmol, 2 eq), NMM (120.29 mg, 1.19 mmol, 130.75 μL, 3 eq) and Intermediate 1-6 (233.76 mg, 396.40 μmol, 1 eq). The mixture was stirred at 25° C. for 1 hr. The reaction mixture was quenched by addition of H₂O (5 mL) at 25° C., and then diluted with EA (5 mL), and extracted with EA (5 mL*3), dried by Na₂SO₄, filtered and concentrated under reduced pressure to give a residue. The residue was purified by prep-HPLC (column: CD04-Welch Utimate C18 150*25*7 um; mobile phase: [water(TFA)-ACN]; gradient: 22%-52% B over 10 min) and concentrated under reduced pressure to give a residue. The residue was further purified by prep-HPLC (basic condition). The eluent was concentrated under reduced pressure to remove ACN and lyophilized to give I-419 (22.29 mg, 27.05 μmol, 6.82% yield, 100% purity) as a yellow solid. LCMS (Method E): Rt=0.421 min, [M+H]⁺=824.5. ¹H NMR (400 MHz, METHANOL-d4) δ=9.08 (d, J=2.0 Hz, 1H), 8.56 (d, J=2.0 Hz, 1H), 8.37 (d, J=7.6 Hz, 1H), 7.94 (d, J=6.4 Hz, 1H), 7.90-7.81 (m, 2H), 7.60 (s, 1H), 7.56 (d, J=8.0 Hz, 1H), 7.50-7.43 (m, 2H), 7.40-7.32 (m, 2H), 7.20-7.12 (m, 1H), 4.68 (d, J=14.0 Hz, 2H), 4.58 (s, 1H), 4.42-4.37 (m, 1H), 4.39 (s, 1H), 3.84-3.63 (m, 5H), 3.63-3.62 (m, 1H), 3.61-3.47 (m, 4H), 3.21 (s, 2H), 3.01-2.93 (m, 1H), 2.76 (q, J=7.6 Hz, 2H), 2.52 (d, J=2.4 Hz, 4H), 2.53-2.48 (m, 1H), 2.29-2.21 (m, 2H), 1.99-1.89 (m, 2H), 1.84-1.75 (m, 1H), 1.29 (t, J=7.6 Hz, 7H).

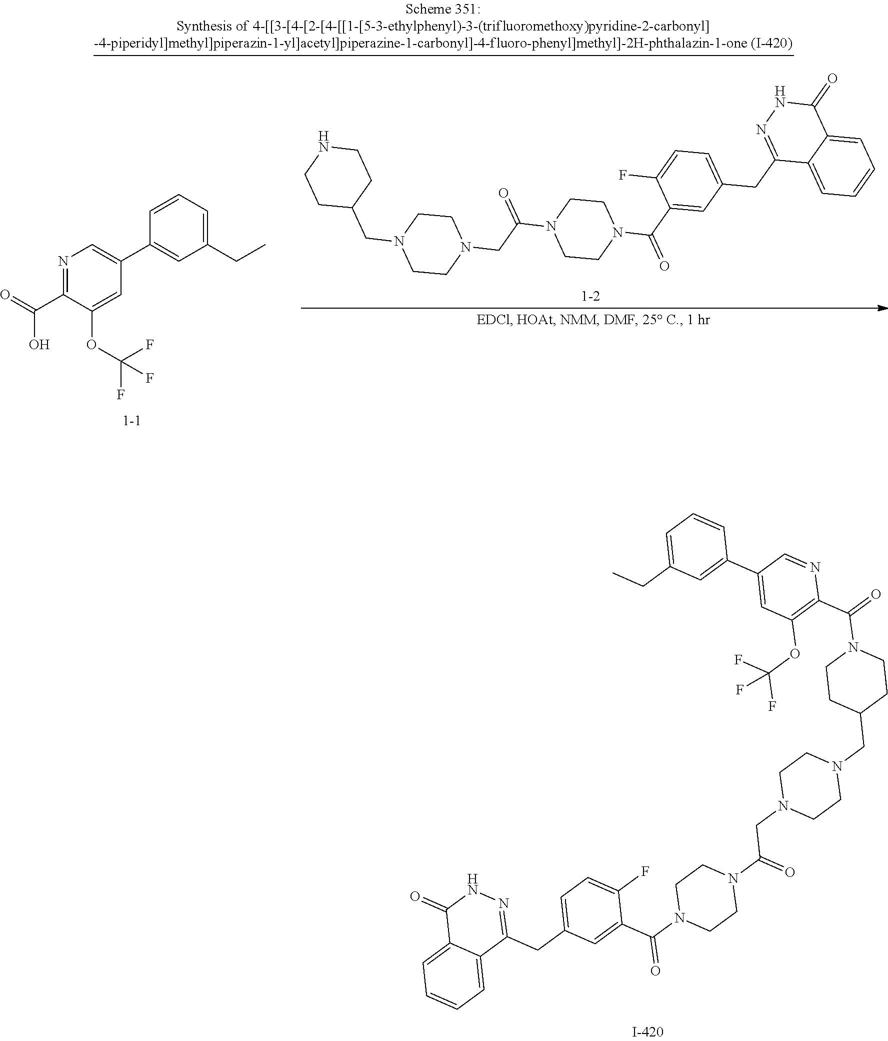 [SMILES: CCc1cccc(-c2cnc(C(=O)O)c(OC(F)(F)F)c2)c1 ; O=C(CN1CCN(CC2CCNCC2)CC1)N1CCN(C(=O)c2cc(Cc3n[nH]c(=O)c4ccccc34)ccc2F)CC1 ; CCc1cccc(-c2cnc(C(=O)N3CCC(CN4CCN(CC(=O)N5CCN(C(=O)c6cc(Cc7n[nH]c(=O)c8ccccc78)ccc6F)CC5)CC4)CC3)c(OC(F)(F)F)c2)c1]

A solution of Intermediate 1-1 (38.39 mg, 123.33 μmol, 1 eq) in DMF (1 mL) and NMM (62.37 mg, 616.64 μmol, 67.80 μL, 5 eq) was stirred at 25° C. for 10 min. Then to the mixture was added Intermediate 1-2 (80 mg, 135.66 μmol, 1.1 eq), EDCI (70.93 mg, 369.99 μmol, 3 eq), and HOAt (16.79 mg, 123.33 μmol, 17.25 μL, 1 eq) and the mixture was stirred at 25° C. for 50 min. The mixture was filtered. The filtrate was purified by reversed-phase HPLC (0.1% FA condition). The eluent was concentrated under reduced pressure to remove ACN and then lyophilized to give I-420 (5.53 mg, 5.85 μmol, 4.74% yield, 98.2% purity, FA salt) as a yellow solid. LCMS (Method D): Rt: 0.405 min, (M+H)=883.5 ¹H NMR (400 MHz, METHANOL-d4) δ=8.86 (d, J=1.6 Hz, 1H), 8.56-8.43 (m, 1H), 8.39-8.34 (m, 1H), 8.11-8.07 (m, 1H), 7.99-7.93 (m, 1H), 7.91-7.80 (m, 2H), 7.58-7.42 (m, 4H), 7.40-7.32 (m, 2H), 7.20-7.13 (m, 1H), 4.85-4.79 (m, 1H), 4.72-4.64 (m, 1H), 4.39 (s, 2H), 3.85-3.61 (m, 4H), 3.58-3.44 (m, 3H), 3.37-3.32 (m, 2H), 3.30-3.26 (m, 2H), 3.20-3.09 (m, 1H), 3.00-2.86 (m, 1H), 2.79-2.72 (m, 2H), 2.70-2.51 (m, 7H), 2.43-2.28 (m, 2H), 1.94 (s, 2H), 1.82-1.70 (m, 1H), 1.33-1.17 (m, 5H). ¹⁹F NMR (376 MHz, METHANOL-d4) δ=−58.723, −120.712.

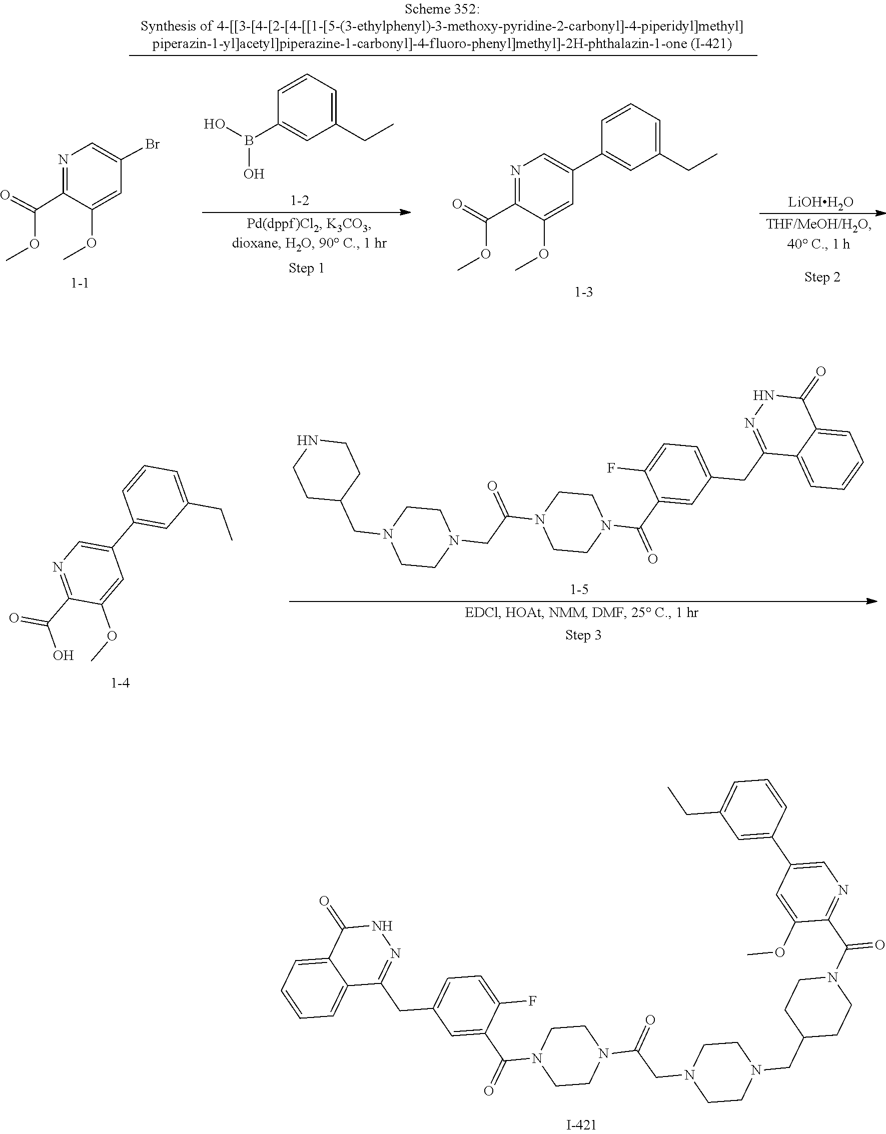

### Step 1: Synthesis of Intermediate 1-3

To a solution of Intermediate 1-1 (150 mg, 609.61 μmol, 1 eq) and Intermediate 1-2 (91.43 mg, 609.61 μmol, 1 eq) in dioxane (1 mL) and H₂O (0.25 mL) was added K₂CO₃ (252.76 mg, 1.83 mmol, 3 eq) and Pd(dppf)Cl₂ (44.61 mg, 60.96 μmol, 0.1 eq). The suspension was degassed and purged with N₂ 3 times. The mixture was stirred at 90° C. for 1 hr under N₂. The reaction mixture was diluted with water (21 mL) and extracted with ethyl acetate (7 mL×3). The combined organic layers were washed with brine (7 mL×3) and dried over sodium sulfate, filtered and concentrated under reduced pressure to give a residue. The residue was purified by flash silica gel chromatography (ISCO; 12 g SepaFlash Silica Flash Column, Eluent of 30˜50% Ethyl acetate/Petroleum ether gradient @40 mL/min). The eluent was concentrated under reduced pressure to give Intermediate 1-3 (140 mg, 516.01 μmol, 84.65% yield, 100% purity) as a brown solid. LCMS (Method D): Rt: 0.426 min, (M+H)=272.0. ¹H NMR (400 MHz, CHLOROFORM-d) δ=8.51 (s, 1H), 7.51 (s, 1H), 7.45-7.39 (m, 3H), 7.35-7.28 (m, 1H), 4.02 (d, J=4.0 Hz, 6H), 2.78-2.73 (m, 2H), 1.35-1.28 (m, 3H).

### Step 2: Synthesis of Intermediate 1-4

To a solution of Intermediate 1-3 (140 mg, 516.01 μmol, 1 eq) in THF (0.5 mL), MeOH (0.5 mL) and H₂O (0.2 mL) was added LiOH·H₂O (64.96 mg, 1.55 mmol, 3 eq). The mixture was stirred at 40° C. for 1 hr. The mixture was concentrated to give a residue. The reaction mixture was diluted with water (9 mL). Then the mixture was adjusted to pH=6˜7 with a saturated citric acid aqueous solution. Then the reaction mixture was extracted with DCM (3 mL×3). The combined organic layers were washed with brine (3 mL×3) and dried over sodium sulfate, filtered and concentrated under reduced pressure to give Intermediate 1-4 (100 mg, crude) as a brown oil. LCMS (Method D): Rt: 0.344 min, (M+H)=257.9.

### Step 3: Synthesis of I-421

To a solution of Intermediate 1-3 (50 mg, 194.34 μmol, 1 eq) in DMF (1 mL) was added EDCI (111.76 mg, 583.01 μmol, 3 eq), NMM (98.28 mg, 971.69 μmol, 106.83 μL, 5 eq) and HOAt (26.45 mg, 194.34 μmol, 27.19 μL, 1 eq). The mixture was stirred at 25° C. for 0.5 hr. Then Intermediate 1-2 (114.60 mg, 194.34 μmol, 1 eq) was added into the mixture and it was stirred at 25° C. for 0.5 hr. The reaction mixture was diluted with water (30 mL) and extracted with ethyl acetate (10 mL×3). The combined organic layers were washed with brine (10 mL×3) and dried over sodium sulfate, filtered and concentrated under reduced pressure to give a residue. The crude product was purified by reversed-phase HPLC (0.1% FA condition). The eluent was concentrated under reduced pressure to remove ACN and then lyophilized to give I-421 (14.54 mg, 16.58 μmol, 8.53% yield, 99.78% purity, FA salt) as a white solid. LCMS (Method D): Rt: 0.372 min, (M+H)=829.4. ¹H NMR (400 MHz, METHANOL-d4) δ=8.48 (s, 1H), 8.42-8.33 (m, 2H), 7.98-7.92 (m, 1H), 7.91-7.79 (m, 2H), 7.74 (d, J=1.2 Hz, 1H), 7.57-7.46 (m, 3H), 7.43-7.41 (m, 1H), 7.40-7.34 (m, 1H), 7.31 (d, J=7.6 Hz, 1H), 7.16-7.14 (m, 1H), 4.84 (s, 1H), 4.68 (d, J=12.4 Hz, 1H), 4.38 (s, 2H), 3.99 (s, 3H), 3.84-3.64 (m, 4H), 3.52 (d, J=2.8 Hz, 2H), 3.46-3.32 (m, 4H), 3.11-3.09 (m, 1H), 2.95-2.83 (m, 1H), 2.82-2.48 (m, 10H), 2.39-2.38 (m, 2H), 1.94 (d, J=7.2 Hz, 2H), 1.74 (d, J=12.4 Hz, 1H), 1.38-1.16 (m, 5H). ¹⁹F NMR (400 MHz, METHAN OL-d4) δ=−120.787.

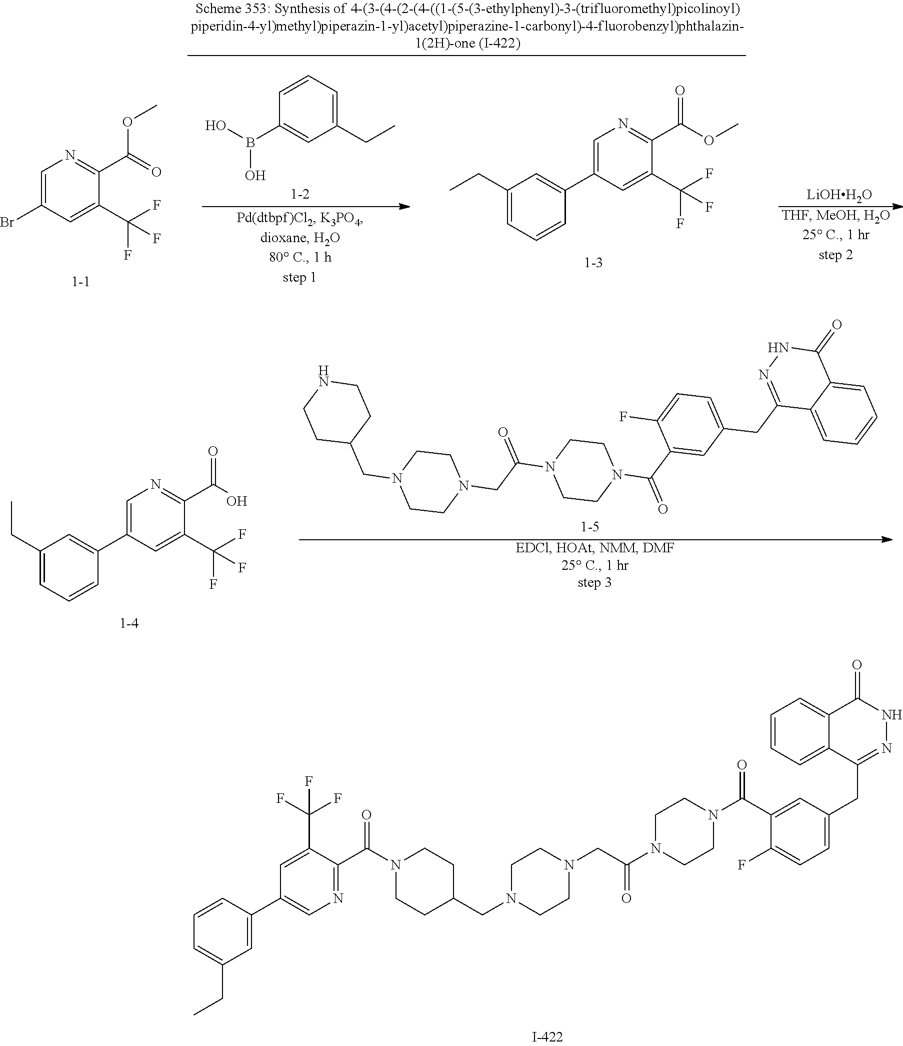

### Step 1: Synthesis of Intermediate 1-3

To a solution of Intermediate 1-1 (250 mg, 880.19 μmol, 1 eq) in dioxane (2.5 mL) and H₂O (0.5 mL) was added ditert-butyl(cyclopentyl) phosphane; dichloropalladium;iron (57.37 mg, 88.02 μmol, 0.1 eq) and K₃PO₄ (560.50 mg, 2.64 mmol, 3 eq) and (3-ethylphenyl)boronic acid (158.42 mg, 1.06 mmol, 1.2 eq). The mixture was stirred at 80° C. for 1 hr. The mixture was washed with water (3 mL) and extracted with EA (2 mL*3). The combined organic layers were dried over sodium sulfate, filtered and concentrated under reduced pressure to give a residue. The residue was purified by flash silica gel chromatography (ISCO; 20 g SepaFlash Silica Flash Column, Eluent of 0˜10% PE/EA gradient @50 mL/min) and then the eluent was concentrated in vacuo to give Intermediate 1-3 (308 mg, crude) as a brown oil. LCMS (Method D): Rt=0.579 min, (M+H)=310.4.

### Step 2: Synthesis of Intermediate 1-4

To a solution of Intermediate 1-3 (308 mg, 995.85 μmol, 1 eq) in MeOH (1 mL), H₂O (1 mL), and THF (1 mL) was added LiOH·H₂O (125.37 mg, 2.99 mmol, 3 eq). The mixture was stirred at 25° C. for 1 hr. The pH was acidified to 3˜4 with 1N HCl, and the mixture was washed with water (5 mL) and extracted with EA (8 mL*3). The combined organic layers were dried over sodium sulfate, filtered and concentrated under reduced pressure to give a residue. The residue was purified by flash silica gel chromatography (ISCO; 20 g SepaFlash Silica Flash Column, Eluent of 0˜70% PE/EA gradient @50 mL/min) and then the eluent was concentrated in vacuo to give Intermediate 1-4 (269 mg, 911.07 μmol, 91.49% yield) as a yellow solid. LCMS (Method D): Rt=0.505 min, (M+H)=296.0. ¹H NMR (400 MHz, METHANOL-d4) δ=9.05 (d, J=2.0 Hz, 1H), 8.42 (d, J=1.6 Hz, 1H), 7.62-7.52 (m, 2H), 7.46 (m, 1H), 7.35 (d, J=7.6 Hz, 1H), 2.75 (m, 2H), 1.29 (m, 3H). ¹⁹F NMR (376 MHz, METHANOL-d4) δ=−61.49.

### Step 3: Synthesis of I-422

To a solution of Intermediate 1-4 (40 mg, 135.48 μmol, 1 eq) in DMF (1 mL) was added HOAt (18.44 mg, 135.48 μmol, 18.95 μL, 1 eq), EDCI (77.91 mg, 406.43 μmol, 3 eq), NMM (68.51 mg, 677.38 mol, 74.47 μL, 5 eq) and Intermediate 1-5 (103.86 mg, 165.86 μmol, 1.22 eq, HCl salt). The mixture was stirred at 25° C. for 1 hr. The mixture was washed with water (1 mL) and extracted with EA (2 mL*3). The combined organic layers were dried over sodium sulfate, filtered and concentrated under reduced pressure to give a residue. The residue was purified by prep-HPLC (FA condition), the eluent was concentrated under reduced pressure to remove ACN and lyophilized to give I-422 (14.11 mg, 15.32 μmol, 11.31% yield, 99.127% purity, FA salt) as white solid. LCMS (Method D): Rt=0.392 min, (M+H)=867.4. ¹H NMR (400 MHz, METHANOL-d4) δ=9.08 (d, J=1.6 Hz, 1H), 8.56-8.44 (m, 1H), 8.42 (d, J=2.0 Hz, 1H), 8.37 (d, J=6.8 Hz, 1H), 7.99-7.91 (m, 1H), 7.90-7.81 (m, 2H), 7.61-7.55 (m, 2H), 7.51-7.44 (m, 2H), 7.40-7.33 (m, 2H), 7.16 (m, 1H), 4.73-4.64 (m, 1H), 4.59 (s, 1H), 4.39 (s, 2H), 3.80-3.66 (m, 4H), 3.56-3.50 (m, 2H), 3.37 (d, J=2.4 Hz, 3H), 3.26-3.23 (m, 1H), 3.13 (m, 1H), 2.97-2.89 (m, 1H), 2.76 (d, J=7.6 Hz, 2H), 2.68-2.43 (m, 8H), 2.35-2.25 (m, 2H), 1.98-1.88 (m, 2H), 1.77-1.68 (m, 1H), 1.32-1.28 (m, 3H), 1.27-1.14 (m, 2H). ¹⁹F NMR (376 MHz, METHANOL-d4) δ=−48.77, −128.65.

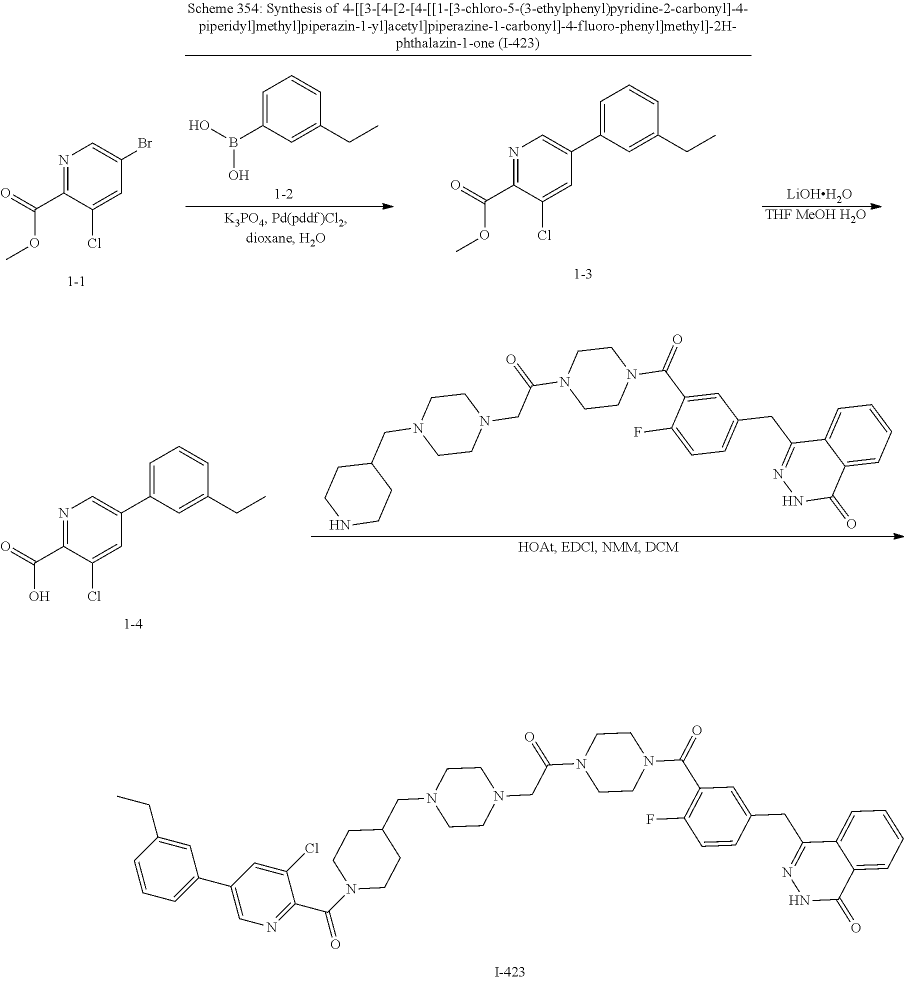

### Step 1: Synthesis of Intermediate 1-3.

A mixture of Intermediate 1-2 (164.67 mg, 1.10 mmol, 1.1 eq), Intermediate 1-1 (250 mg, 998.10 μmol, 1 eq), K₃PO₄ (635.59 mg, 2.99 mmol, 3 eq), cyclopentyl (diphenyl) phosphane;dichloropalladium;iron (73.03 mg, 99.81 μmol, 0.1 eq) in dioxane (3 mL) and H₂O (0.3 mL) was degassed and purged with N₂ 3 times, and then the mixture was stirred at 80° C. for 1 hr under N₂ atmosphere. The reaction mixture was diluted with H₂O (6 mL) and extracted with EA (6 mL*3). The combined organic layers were dried over Na₂SO₄, filtered and concentrated under reduced pressure to give a residue. The residue was purified by flash silica gel chromatography (ISCO; 20 g SepaFlash Silica Flash Column, Eluent of 90% EA/PE gradient @80 mL/min). The eluent was concentrated under reduced pressure to give Intermediate 1-3 (260 mg, 826.73 μmol, 82.83% yield, 87.675% purity) as a black solid. LCMS (Method D): Retention time: 0.558 min, (M+H)=276.2. ¹H NMR (400 MHz, CHLOROFORM-d) δ==8.74-8.68 (m, 1H), 8.15-8.13 (m, 1H), 7.54-7.42 (m, 2H), 7.42-7.34 (m, 1H), 7.29 (d, J=6.0 Hz, 1H), 3.96 (d, J=1.6 Hz, 3H), 2.74-2.65 (m, 2H), 1.27-1.22 (m, 3H).

### Step 2: Synthesis of Intermediate 1-4.

To a solution of Intermediate 1-3 (150 mg, 544.01 μmol, 1 eq) in THF (0.5 mL), MeOH (0.5 mL) and H₂O (0.5 mL) was added LiOH·H₂O (68.49 mg, 1.63 mmol, 3 eq). The mixture was stirred at 25° C. for 1 hr. The reaction mixture was quenched by addition of H₂O (4 mL), and extracted with EA (4 mL*3). The aqueous phase was adjusted to pH 3 wit 1M HCl and extracted with EA (4 mL*3), dried over Na₂SO₄, filtered and concentrated under reduced pressure to give a residue. Without further purification, it was used directly for the next reaction. Intermediate 1-4 (135 mg, 471.11 μmol, 86.60% yield, 91.326% purity) was obtained as a white solid. LCMS (Method F): Retention time: 0.412 min, (M+H)=262.1. ¹H NMR (400 MHz, DMSO-d6) δ=8.78 (d, J=2.0 Hz, 1H), 8.22 (d, J=2.0 Hz, 1H), 7.57-7.48 (m, 2H), 7.45-7.41 (m, 1H), 7.33 (d, J=7.6 Hz, 1H), 2.79-2.68 (m, 2H), 1.31-1.26 (m, 3H).

### Step 3: Synthesis of I-423

To a solution of Intermediate 1-4 (100 mg, 382.11 μmol, 1 eq) in DMF (10 mL) was added HOAT (52.01 mg, 382.11 μmol, 53.45 μL, 1 eq), EDCI (219.75 mg, 1.15 mmol, 3 eq), NMM (193.25 mg, 1.91 mmol, 210.06 μL, 5 eq) and Intermediate 1-5 (225.33 mg, 382.11 μmol, 1 eq). The mixture was stirred at 25° C. for 1 hr. The reaction mixture was diluted with H₂O (20 mL) and extracted with EA (10 mL*3). The combined organic layers were washed by brine (5 mL*3), dried over Na₂SO₄, filtered and concentrated under reduced pressure to give a residue. The residue was purified by prep-HPLC (FA condition). The eluent was concentrated and lyophilized to give I-423 (68.57 mg, 76.36 μmol, 19.98% yield, 97.929% purity, FA salt) as a white solid. LCMS (Method D): Retention time: 0.395 min, (M+H)=833.5. ¹H NMR (400 MHz, CHLOROFORM-d) δ=8.79 (d, J=1.6 Hz, 1H), 8.43 (s, 1H), 8.37 (d, J=7.6 Hz, 1H), 8.23 (d, J=1.6 Hz, 1H), 7.98-7.92 (m, 1H), 7.91-7.80 (m, 2H), 7.57-7.47 (m, 3H), 7.46-7.41 (m, 1H), 7.40-7.30 (m, 2H), 7.21-7.12 (m, 1H), 4.84-4.82 (m, 1H), 4.69 (d, J=12.8 Hz, 1H), 4.38 (s, 2H), 3.82-3.71 (m, 2H), 3.68 (s, 2H), 3.52 (s, 2H), 3.40 (s, 4H), 3.20-3.11 (m, 1H), 3.01-2.85 (m, 1H), 2.80-2.71 (m, 3H), 2.67 (d, J=15.2 Hz, 7H), 2.47-2.35 (m, 2H), 1.96 (d, J=6.8 Hz, 2H), 1.76 (d, J=12.8 Hz, 1H), 1.33-1.22 (m, 5H). ¹⁹F NMR (376 MHz, CHLOROFORM-d) δ=−117.59.

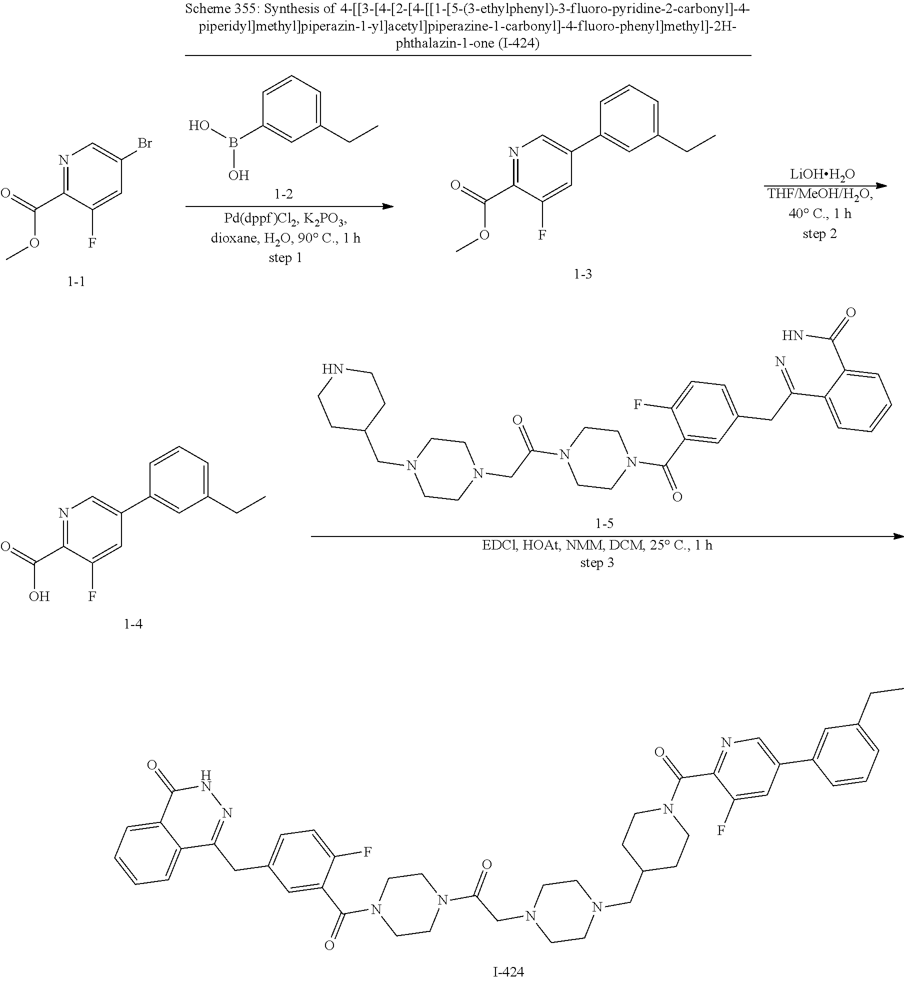

### Step 1: Synthesis of Intermediate 1-3

To a solution of Intermediate 1-1 (200 mg, 854.62 μmol, 1 eq) and Intermediate 1-2 (128.18 mg, 854.62 μmol, 1 eq) in H₂O (0.6 mL) and dioxane (2 mL) was added cyclopentyl (diphenyl) phosphane; dichloropalladium; iron (62.53 mg, 85.46 μmol, 0.1 eq) and dipotassium; carbonate (354.34 mg, 2.56 mmol, 3 eq). The suspension was stirred at 90° C. for 1 hr under N₂. The reaction mixture was diluted with water (9 mL) and extracted with ethyl acetate (3 mL×3). The combined organic layers were washed with brine (3 mL×3) and dried over sodium sulfate, filtered and concentrated under reduced pressure to give a residue. The residue was purified by flash silica gel chromatography (ISCO; 12 g SepaFlash Silica Flash Column, Eluent of 0˜10% Petroleum ether: Ethyl acetate gradient @40 mL/min). The eluent was concentrated under reduced pressure to give Intermediate 1-3 (200 mg, 758.27 μmol, 88.73% yield, 98.3% purity) as a yellow oil. LCMS (Method D): Rt: 0.444 min, (M+H)=724.1. ¹H NMR (400 MHz, CHLOROFORM-d) δ=8.79 (s, 1H), 7.76-7.72 (m, 1H), 7.50-7.41 (m, 3H), 7.39-7.30 (m, 1H), 4.05 (s, 3H), 2.78-2.72 (m, 2H), 1.32-1.24 (m, 3H). ¹⁹F NMR (376 MHz, METHANOL-d4) δ=−117.469.

### Step 2: Synthesis of Intermediate 1-4

To a solution of Intermediate 1-4 (200 mg, 771.38 μmol, 1 eq) in THF (1.6 mL) and H₂O (0.4 mL) was added LiOH·H₂O (32.37 mg, 771.38 μmol, 1 eq). The mixture was stirred at 50° C. for 1 hr. The pH of the reaction solution was adjusted to pH 6.5 with citric acid monohydrate. The reaction mixture was diluted with water (21 mL) and extracted with ethyl acetate (7 mL×3). The combined organic layers were washed with brine (7 mL×3) and dried over sodium sulfate, filtered and concentrated under reduced pressure to give Intermediate 1-4 (150 mg, 601.84 μmol, 78.02% yield, 98.4% purity) as a white solid. LCMS (Method D): Rt: 0.400 min, (M+H)=246.0.

### Step 3: Synthesis of I-424

To a solution of Intermediate 1-4 (204.25 mg, 326.20 μmol, 1 eq, HCl salt) in DCM (2 mL) was added EDCI (187.60 mg, 978.60 μmol, 3 eq), HOAt (44.40 mg, 326.20 μmol, 45.63 μL, 1 eq) and NMM (164.97 mg, 1.63 mmol, 179.32 μL, 5 eq) at 25° C. for 0.5 hr. Then Intermediate 1-5 (80 mg, 326.20 μmol, 1 eq) was added into the mixture and it was stirred at 25° C. for 0.5 hr. The reaction mixture was diluted with water (18 mL) and extracted with ethyl acetate (6 mL×3). The combined organic layers were washed with brine (6 mL×3) and dried over sodium sulfate, filtered and concentrated under reduced pressure to give a residue. The crude product was purified by reversed-phase HPLC (column: CD02-Waters X bridge BEH C18 150*25*10 um; mobile phase: [water (NH₄HCO₃)-ACN]; gradient: 33%-63% B over 10 min) and the eluent was concentrated under reduced pressure to remove ACN and then lyophilized to give I-424 (14.66 mg, 17.95 μmol, 5.50% yield, 100% purity) as a yellow oil. LCMS (Method D): Rt: 0.390 min, (M+H)=817.3. ¹H NMR (400 MHz, CHLOROFORM-d) δ=9.93 (s, 1H), 8.66 (s, 1H), 8.51-8.43 (m, 1H), 7.79 (d, J=3.6 Hz, 2H), 7.72 (s, 2H), 7.43 (s, 3H), 7.38-7.28 (m, 3H), 7.12-7.02 (m, 1H), 4.85-4.76 (m, 1H), 4.29 (s, 2H), 3.95-3.43 (m, 8H), 3.39-3.19 (m, 4H), 3.15-3.07 (m, 1H), 2.96-2.69 (m, 5H), 2.67-2.13 (m, 7H), 2.04-1.74 (m, 3H), 1.37-1.25 (m, 5H). ¹⁹F NMR (376 MHz, METHANOL-d4) δ=−117.589, 124.952.

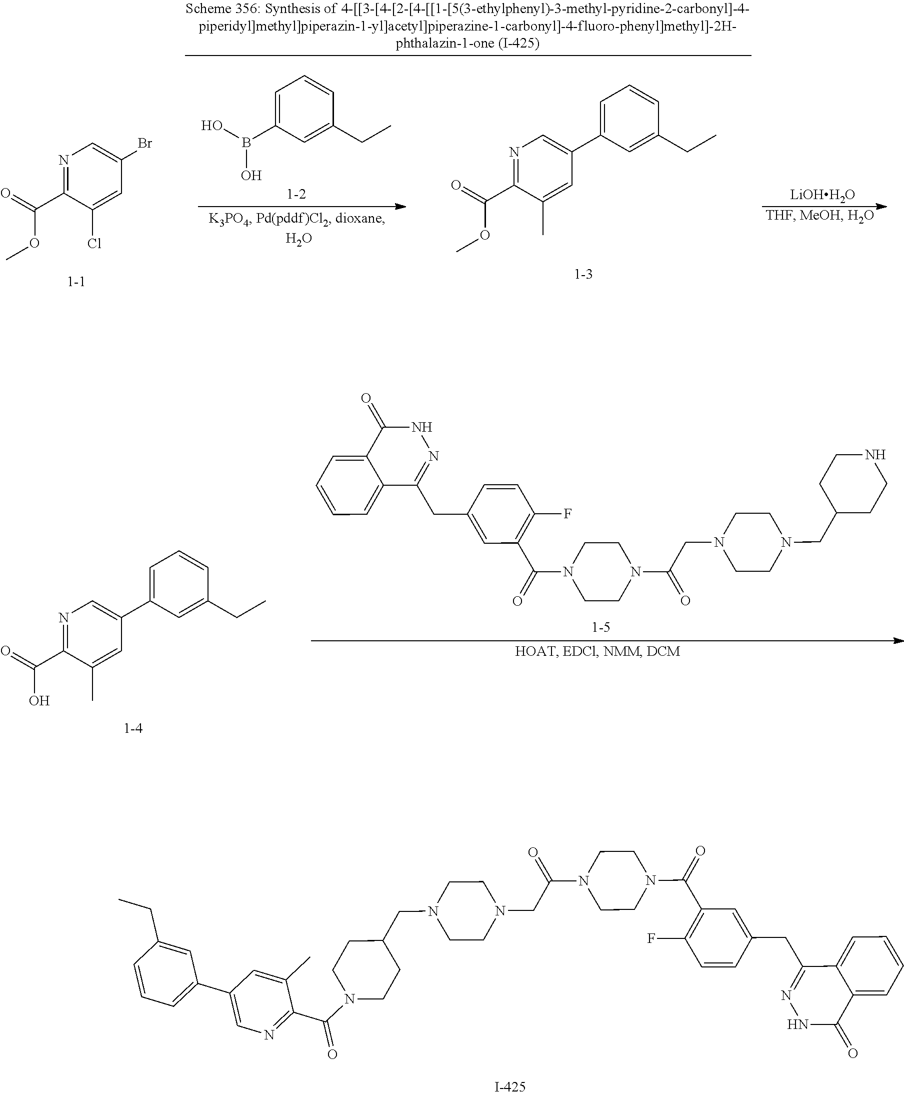

### Step 1: Synthesis of Intermediate 1-3.

A mixture of Intermediate 1-2 (179.28 mg, 1.20 mmol, 1.1 eq), Intermediate 1-1 (250 mg, 1.09 mmol, 1 eq), K₃PO₄ (692.00 mg, 3.26 mmol, 3 eq), cyclopentyl (diphenyl) phosphane; dichloropalladium;iron (79.51 mg, 108.67 μmol, 0.1 eq) in dioxane (3 mL) and H₂O (0.3 mL) was degassed and purged with N₂ 3 times, and then the mixture was stirred at 80° C. for 1 hr under N₂ atmosphere. The reaction mixture was diluted with H₂O (6 mL) and extracted with EA (6 mL*3). The combined organic layers were dried over Na₂SO₄, filtered and concentrated under reduced pressure to give a residue. The residue was purified by flash silica gel chromatography (ISCO; 20 g SepaFlash Silica Flash Column, Eluent of 90% EA/PE gradient @80 mL/min). The eluent concentrated under reduced pressure to give Intermediate 1-3 (180 mg, 686.10 μmol, 63.14% yield, 97.316% purity) as a white solid. LCMS (Method D): Retention time: 0.513 min, (M+H)=256.2. ¹H NMR (400 MHz, CHLOROFORM-d) δ=8.67 (d, J=2.0 Hz, 1H), 8.01 (d, J=1.6 Hz, 1H), 7.54-7.47 (m, 2H), 7.43-7.39 (m, 1H), 7.30 (d, J=7.6 Hz, 1H), 3.96 (s, 3H), 2.76-2.70 (m, 2H), 2.63 (s, 3H), 1.30-1.26 (m, 3H).

### Step 2: Synthesis of Intermediate 1-4.

To a solution of Intermediate 1-3 (100 mg, 391.68 μmol, 1 eq) in THF (0.5 mL), MeOH (0.5 mL), and H₂O (0.5 mL). H₂O (4 mL) was added and the mixture was extracted with EA (4 mL*3). The aqueous phase was adjusted to pH 3 with 1M HCl and was extracted with EA (4 mL*3), dried over Na₂SO₄, filtered and concentrated under reduced pressure to give a residue. Without further purification, the crude was used for the next reaction. Intermediate 1-4 (90 mg, 360.44 μmol, 92.03% yield, 96.633% purity) was obtained as a white solid. LCMS (Method F): Retention time: 0.342 min, (M+H)=242.1. ¹H NMR (400 MHz, DMSO-d6) δ=8.69 (d, J=2.0 Hz, 1H), 8.08 (d, J=1.6 Hz, 1H), 7.56-7.48 (m, 2H), 7.43-7.40 (m, 1H), 7.31 (d, J=7.6 Hz, 1H), 2.77-2.71 (m, 2H), 2.70 (s, 3H), 1.30-1.26 (m, 3H).

### Step 3: Synthesis of I-425

To a solution of Intermediate 1-4 (90 mg, 373.00 μmol, 1 eq) in DMF (8 mL) was added HOAT (50.77 mg, 373.00 μmol, 52.18 μL, 1 eq), EDCI (214.52 mg, 1.12 mmol, 3 eq), NMM (188.64 mg, 1.87 mmol, 205.04 μL, 5 eq) and Intermediate 1-5 (219.96 mg, 373.00 μmol, 1 eq). The mixture was stirred at 25° C. for 1 hr. The reaction mixture was diluted with H₂O (16 mL) and extracted with EA (15 mL*3). The combined organic layers were washed by brine (5 mL*3), dried over Na₂SO₄, filtered and concentrated under reduced pressure to give a residue. The residue was purified by prep-HPLC (FA condition). The eluent was concentrated and lyophilized to give I-425 (49.3 mg, 56.65 μmol, 15.19% yield, 98.711% purity, FA salt) as a white solid. LCMS (Method D): Retention time: 0.393 min, (M+H)=813.6. ¹H NMR (400 MHz, CHLOROFORM-d) δ=8.63 (d, J=1.6 Hz, 1H), 8.53-8.48 (m, 1H), 8.37 (d, J=7.6 Hz, 1H), 8.01 (s, 1H), 7.98-7.92 (m, 1H), 7.90-7.79 (m, 2H), 7.55-7.45 (m, 3H), 7.44-7.34 (m, 2H), 7.29 (d, J=7.2 Hz, 1H), 7.20-7.12 (m, 1H), 4.85-4.83 (m, 1H), 4.70 (d, J=12.0 Hz, 1H), 4.38 (s, 2H), 3.79 (d, J=2.8 Hz, 1H), 3.76-3.62 (m, 3H), 3.57-3.43 (m, 2H), 3.41-3.32 (m, 3H), 3.25 (s, 1H), 3.17-3.06 (m, 1H), 2.97-2.87 (m, 1H), 2.83-2.72 (m, 2H), 2.57 (s, 8H), 2.39 (s, 3H), 2.35-2.27 (m, 2H), 1.92 (s, 2H), 1.79-1.68 (m, 1H), 1.32-1.27 (m, 3H), 1.26-1.09 (m, 2H). ¹⁹F NMR (376 MHz, CHLOROFORM-d) δ=−120.78.

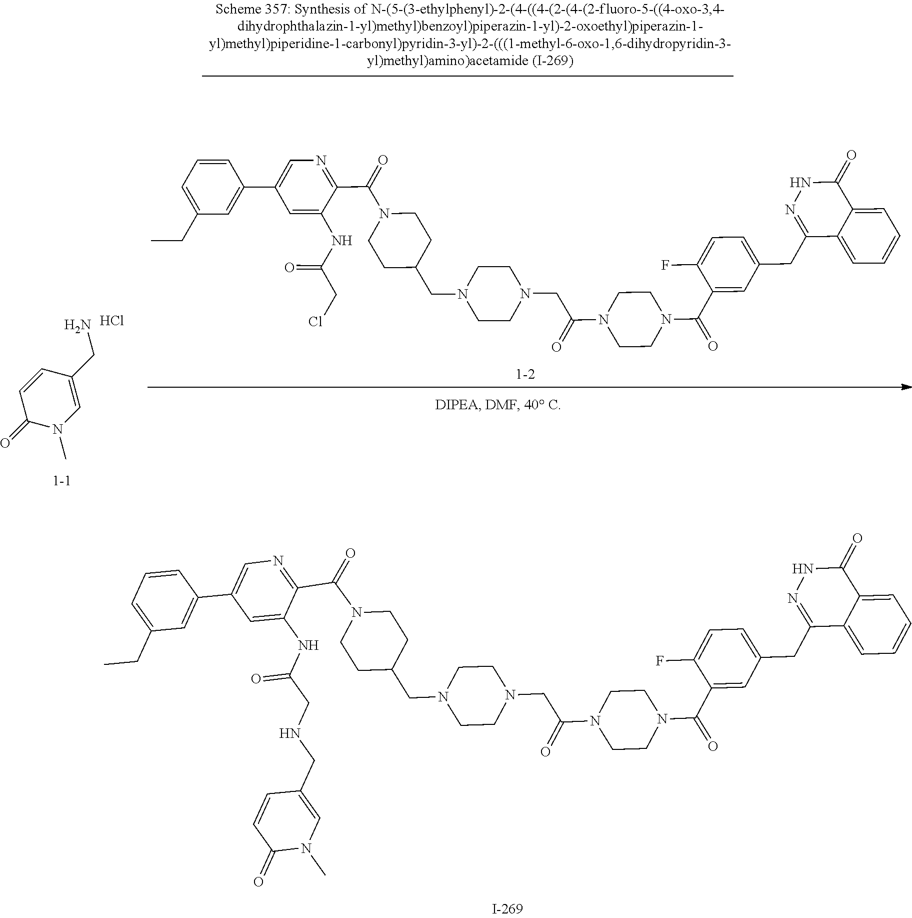

To a solution of Intermediate 1-2 (100 mg, 112.30 μmol, 1 eq) and Intermediate 1-1 (19.61 mg, 112.30 μmol, 1 eq, HCl salt) in DMF (1 mL) was added DIEA (43.54 mg, 336.91 μmol, 58.68 μL, 3 eq). The mixture was stirred at 40° C. for 12 h. Without workup, the crude product was purified by prep-HPLC (column: Phenomenex luna C18 150*25 mm*10 um; mobile phase: [water (FA)-ACN]; gradient: 3%-33% B over 8 min) and the eluent was lyophilized to give I-269 (31.1 mg, 30.54 μmol, 27.19% yield, 97.413% purity) as a white solid. LCMS (Method D): Retention time: 0.345 min, [M+H]⁺=992.3. ¹HNMR (400 MHz, METHANOL-d₄) δ=8.76 (s, 1H), 8.60 (d, J=1.2 Hz, 1H), 8.38-8.29 (m, 2H), 7.98-7.91 (m, 1H), 7.90-7.80 (m, 2H), 7.80-7.71 (m, 2H), 7.54-7.48 (m, 3H), 7.43 (t, J=7.6 Hz, 1H), 7.39-7.30 (m, 2H), 7.17 (t, J=8.8 Hz, 1H), 6.55 (d, J=9.2 Hz, 1H), 4.75-4.63 (m, 2H), 4.38 (s, 2H), 3.88-3.74 (m, 3H), 3.67 (s, 4H), 3.58 (s, 3H), 3.56-3.46 (m, 6H), 3.34 (d, J=1.6 Hz, 1H), 3.17 (t, J=12.8 Hz, 1H), 2.99-2.71 (m, 12H), 2.64 (s, 2H), 2.04 (d, J=1.6 Hz, 1H), 1.93 (d, J=12.4 Hz, 1H), 1.77 (d, J=12.4 Hz, 1H), 1.39-1.24 (m, 5H). ¹⁹F NMR (377 MHz, METHANOL-d₄) δ=−120.763.

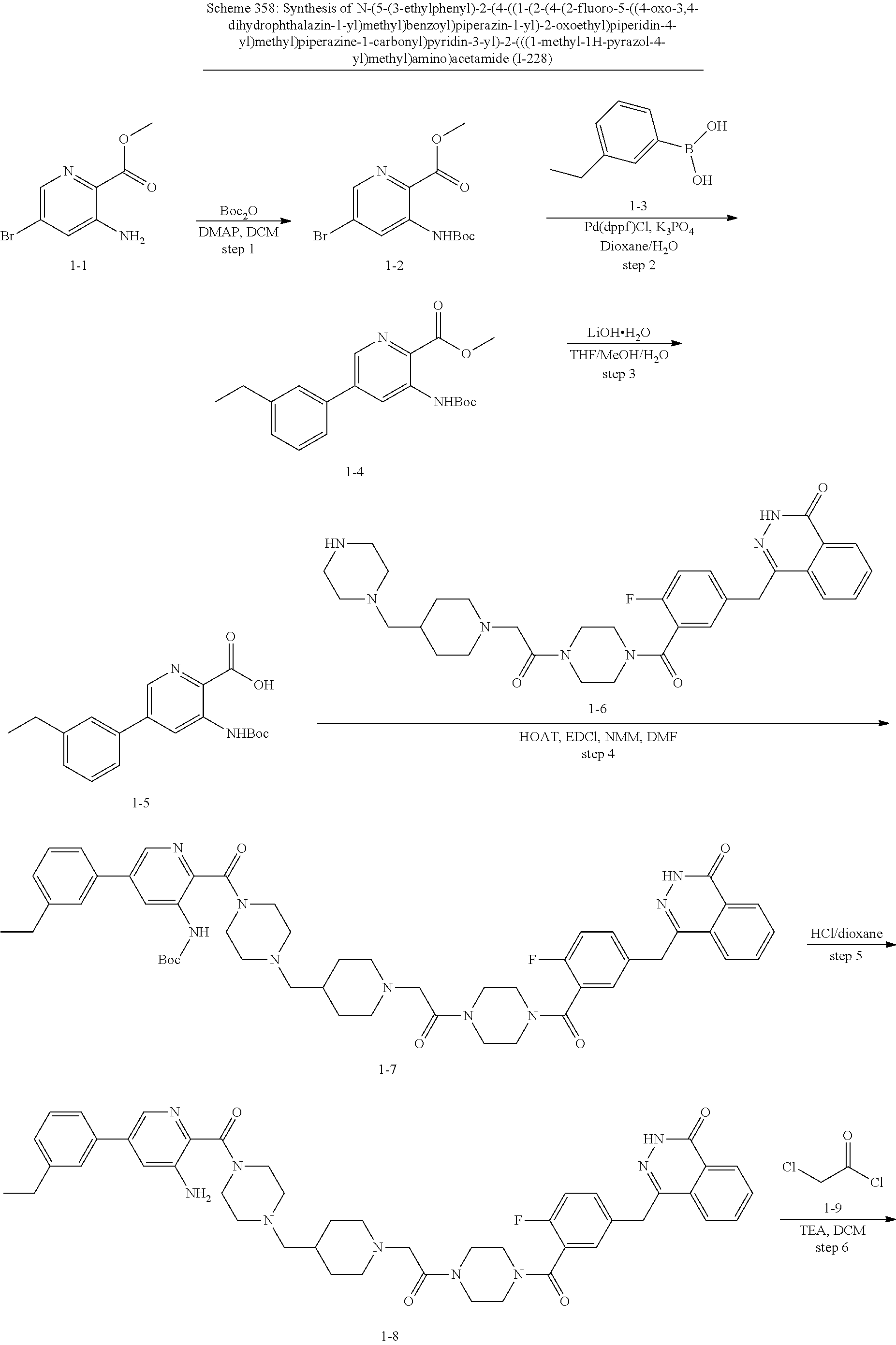

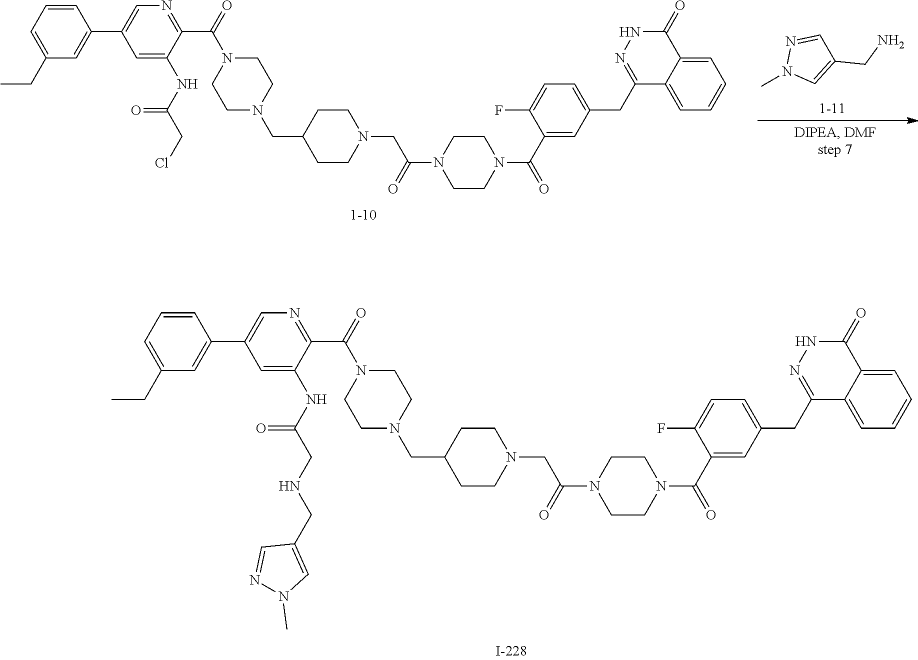

### Step 1: Synthesis of Intermediate 1-2

To a solution of Intermediate 1-1 (20 g, 86.56 mmol, 1 eq) in DCM (200 mL) was added Boc₂O (22.67 g, 103.88 mmol, 23.86 mL, 1.2 eq) and DMAP (1.06 g, 8.66 mmol, 0.1 eq). The mixture was stirred at 25° C. for 2 hrs. Then the mixture was stirred at 25° C. for 10 hrs. The residue was poured into water (100 mL). The aqueous phase was extracted with EA (50 mL*3). The combined organic phase was washed with brine (100 mL*3), dried with anhydrous sodium sulfate, filtered, and concentrated in vacuo to give a residue. The residue was purified by flash silica gel chromatography (ISCO; 40 g SepaFlash Silica Flash Column, Eluent of 0˜20% Ethylacetate/Petroleum ether gradient @100 mL/min) and concentrated under vacuum to afford Intermediate 1-2 (18 g, 54.35 mmol, 62.79% yield) was obtained as a brown solid. LCMS (Method E): Rt=0.602 min, M+H=331.0. ¹H NMR (400 MHz, DMSO-d₆) δ=9.98 (s, 1H), 8.69 (br d, J=2.4 Hz, 1H), 8.42 (br d, J=2.4 Hz, 1H), 3.86 (s, 3H), 1.48 (s, 9H).

### Step 2: Synthesis of Intermediate 1-4

A mixture of Intermediate 1-2 (10 g, 30.20 mmol, 1 eq), Intermediate 1-3 (6.79 g, 45.30 mmol, 1.5 eq), Pd(dppf)Cl₂ (2.21 g, 3.02 mmol, 0.1 eq), and K₃PO₄ (19.23 g, 90.59 mmol, 3 eq) in dioxane (100 mL) and H₂O (20 mL) was degassed and purged with N₂ 3 times, and then the mixture was stirred at 80° C. for 2 hrs under N₂ atmosphere. The residue was poured into water (100 mL). The aqueous phase was extracted with EA (100 mL*3). The combined organic phase was washed with brine (100 mL*3), dried with anhydrous sodium sulfate, filtered, and concentrated to give a residue. The residue was purified by flash silica gel chromatography (ISCO; 220 g SepaFlash Silica Flash Column, Eluent of 0˜20% Ethyl acetate/Petroleum ether gradient @100 mL/min) and concentrated under vacuum to afford Intermediate 1-4 (11 g, crude) as a brown oil. LCMS (Method G): Rt=0.823 min, M+H=357.1. ¹H NMR (400 MHz, DMSO-d₆) δ=10.02 (s, 1H), 8.70 (d, J=1.8 Hz, 1H), 8.60 (d, J=2.0 Hz, 1H), 7.96 (s, 1H), 7.55-7.49 (m, 2H), 7.43 (t, J=7.6 Hz, 1H), 7.34-7.30 (m, 1H), 3.88 (s, 3H), 2.68 (q, J=7.5 Hz, 2H), 1.49 (s, 9H), 1.22 (t, J=7.6 Hz, 3H).

### Step 3: Synthesis of Intermediate 1-5

To a solution of Intermediate 1-4 (5 g, 14.03 mmol, 1 eq) in THF (20 mL), MeOH (10 mL) and H₂O (20 mL) was added LiOH·H₂O (2.94 g, 70.14 mmol, 5 eq). The mixture was stirred at 25° C. for 1 h. The mixture was concentrated under vacuum to remove THF and MeOH. The aqueous phase was adjusted to pH=7 with 1N HCl (35 mL) and filtered and the filter cake was dried under vacuum to give Intermediate 1-5 (4.4 g, crude) as a brown solid. LCMS (Method E): Rt=0.608 min, M+H=343.1. ¹H NMR (400 MHz, DMSO-d₆) δ=12.89 (s, 1H), 8.87 (d, J=1.1 Hz, 1H), 8.36 (d, J=1.6 Hz, 1H), 7.52-7.38 (m, 3H), 7.29 (br d, J=7.4 Hz, 1H), 2.68 (q, J=7.5 Hz, 2H), 1.49 (s, 9H), 1.25-1.15 (m, 3H).

### Step 4: Synthesis of Intermediate 1-7

To a solution of Intermediate 1-5 (100 mg, 292.07 μmol, 1 eq) in DMF (2 mL) was added HOAt (39.75 mg, 292.07 μmol, 40.86 μL, 1 eq), EDCI (111.98 mg, 584.13 μmol, 2 eq), NMM (147.71 mg, 1.46 mmol, 160.55 μL, 5 eq) and Intermediate 1-6 (182.88 mg, 292.07 μmol, 1 eq, HCl salt). The mixture was stirred at 25° C. for 12 hrs. The mixture was poured into 6 ml of H₂O, filtered and the filter cake was dried under vacuum to give Intermediate 1-7 (180 mg, crude) as a brown solid. LCMS (Method E): Rt=0.492 min, M+H=914.5.

### Step 5: Synthesis of Intermediate 1-8

To a solution of Intermediate 1-7 (180 mg, 167.38 μmol, 1 eq) in dioxane (1.5 mL) was added HCl/dioxane (2 M, 83.69 μL, 1 eq). The mixture was stirred at 25° C. for 1 h. The mixture was concentrated under vacuum to give a crude product. The crude product was purified by reversed-phase HPLC (0.1% NH₃·H₂O) to afford Intermediate 1-8 (110 mg, 135.14 μmol, 80.74% yield, 100% purity) as a yellow solid. LCMS (Method E): Rt=0.433 min, M+H=814.5.

### Step 6: Synthesis of Intermediate 1-10

To a solution of Intermediate 1-8 (110 mg, 135.14 μmol, 1 eq) in DCM (2 mL) was added TEA (41.02 mg, 405.43 μmol, 56.43 μL, 3 eq) and Intermediate 1-9 (18.32 mg, 162.17 μmol, 12.92 μL, 1.2 eq). The mixture was stirred at 25° C. for 1 h. The mixture was poured into H₂O (4 mL). The aqueous phase was extracted with DCM (2 mL*3). The combined organic phase was washed with brine (2 mL*3), dried with anhydrous sodium sulfate, filtered, and concentrated to give a residue. The residue was purified by reversed-phase HPLC (0.1% FA condition) to afford Intermediate 1-10 (70 mg, 78.61 μmol, 58.17% yield) as a yellow solid. LCMS (Method E): Rt=0.433 min, M+H=890.3. ¹H NMR (400 MHz, CHLOROFORM-d) δ=10.74 (s, 1H), 10.62 (br d, J=2.8 Hz, 1H), 10.28-10.20 (m, 1H), 8.97 (s, 1H), 8.57 (s, 1H), 8.46 (br d, J=5.1 Hz, 1H), 7.81-7.71 (m, 3H), 7.47-7.38 (m, 3H), 7.36-7.29 (m, 3H), 7.06 (t, J=8.7 Hz, 1H), 4.31-4.20 (m, 4H), 3.87-3.56 (m, 9H), 3.33 (br d, J=14.5 Hz, 3H), 3.11 (q, J=7.4 Hz, 1H), 2.96 (br dd, J=1.8, 6.2 Hz, 2H), 2.60-2.50 (m, 3H), 2.33-2.23 (m, 3H), 1.91-1.60 (m, 10H), 1.32-1.23 (m, 7H).

### Step 7: Synthesis of I-228

To a solution of Intermediate 1-10 (60 mg, 67.38 μmol, 1 eq) in DMF (1 mL) was added DIEA (43.54 mg, 336.91 μmol, 58.68 μL, 5 eq) and Intermediate 1-11 (8.99 mg, 80.86 μmol, 1.2 eq). The mixture was stirred at 40° C. for 2 hrs. 0.5 mL of MeOH was added to the mixture. The mixture was purified by reversed-phase HPLC (0.1% FA condition) to afford I-228 (27.78 mg, 28.78 μmol, 42.72% yield, 100% purity) as a white solid. LCMS (Method E): Rt=0.420 min, M+H=965.4. ¹H NMR (400 MHz, METHANOL-d₄) δ=8.79 (d, J=2.0 Hz, 1H), 8.61 (d, J=2.0 Hz, 1H), 8.55 (s, 1H), 8.40-8.35 (m, 1H), 7.99-7.93 (m, 1H), 7.91-7.82 (m, 2H), 7.68 (s, 1H), 7.58 (s, 1H), 7.55-7.48 (m, 3H), 7.45-7.36 (m, 2H), 7.32 (d, J=7.6 Hz, 1H), 7.17 (m, 1H), 4.39 (s, 2H), 3.88-3.66 (m, 12H), 3.54 (br s, 4H), 3.46-3.39 (m, 3H), 3.05-2.96 (m, 2H), 2.79-2.73 (m, 2H), 2.53-2.41 (m, 4H), 2.28-2.17 (m, 4H), 1.81 (m, 2H), 1.61 (m, 1H), 1.40-1.38 (m, 2H), 1.33-1.24 (m, 5H).

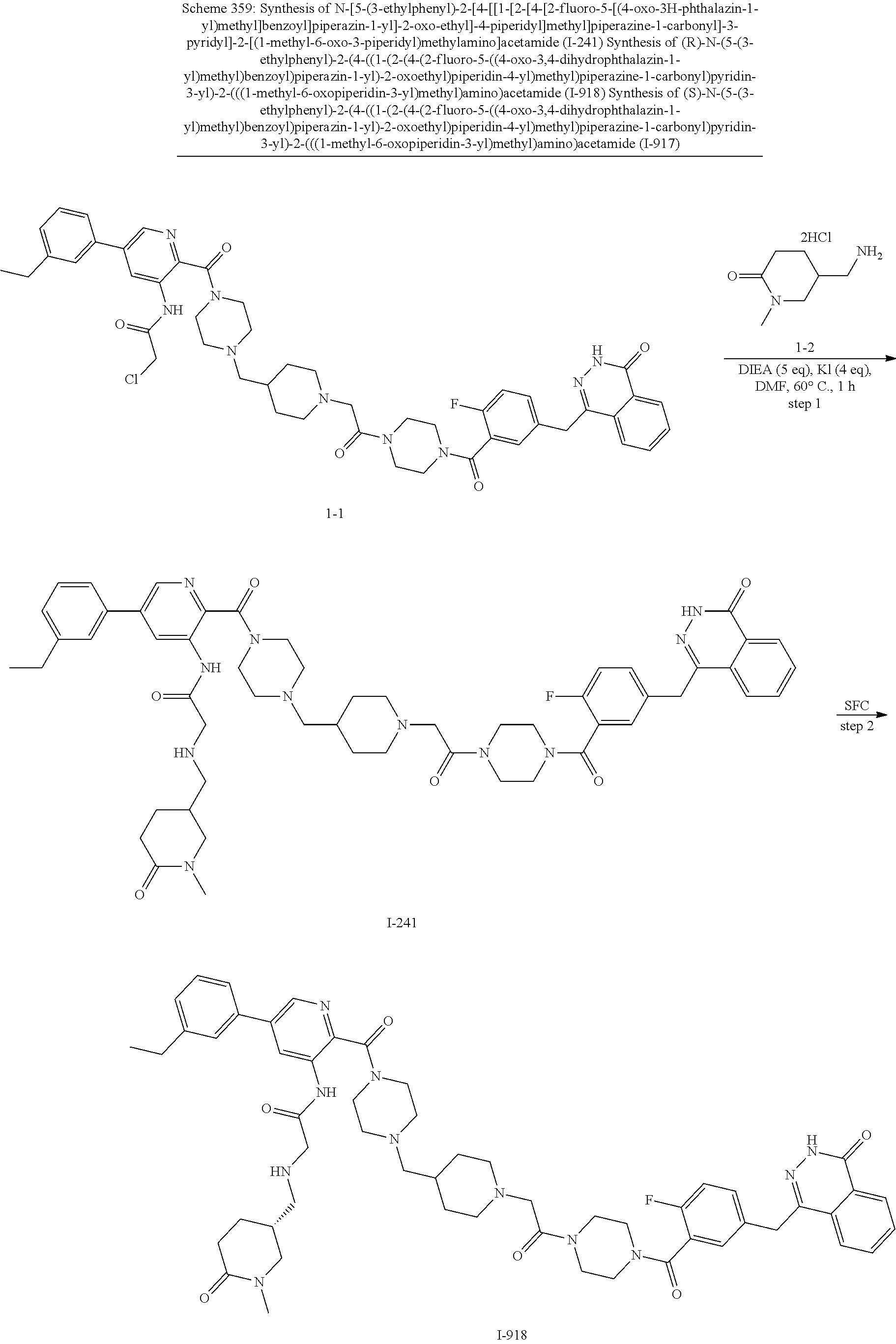

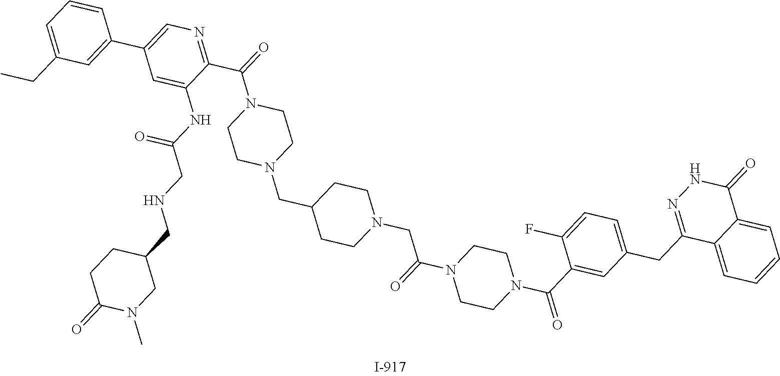 [SMILES: CCc1cccc(-c2cnc(C(=O)N3CCN(CC4CCN(CC(=O)N5CCN(C(=O)c6cc(Cc7n[nH]c(=O)c8ccccc78)ccc6F)CC5)CC4)CC3)c(NC(=O)CNC[C@@H]3CCC(=O)N(C)C3)c2)c1]

### Step 1: Synthesis of I-241

To a solution of Intermediate 1-1 (50 mg, 56.15 μmol, 1 eq) and Intermediate 1-2 (15.05 mg, 84.23 μmol, 1.5 eq, HCl salt) in DMF (0.5 mL) was added DIEA (36.29 mg, 280.76 μmol, 48.90 μL, 5 eq) and KI (37.29 mg, 224.61 μmol, 4 eq). The mixture was stirred at 60° C. for 1 hr. The reaction mixture was concentrated under reduced pressure to give a residue. The residue was purified by prep-HPLC (column: Phenomenex luna C18 150*25 mm*10 um; mobile phase: [water (TFA)-ACN]; gradient: 11%-41% B over 15 min), then concentrated to remove organic solvents and lyophilized to give I-241 (37 mg, 37.14 μmol, 66.15% yield, 100% purity) as a white solid. LCMS (Method E): Rt=0.414 min, [M+H]⁺=996.5. SFC: Rt=4.967 min, 5.469 min. ¹H NMR (400 MHz, METHANOL-d₄) δ=8.72 (s, 1H), 8.37 (d, J=7.6 Hz, 1H), 8.21 (d, J=1.6 Hz, 1H), 7.99-7.92 (m, 1H), 7.91-7.80 (m, 2H), 7.56-7.41 (m, 4H), 7.40-7.32 (m, 2H), 7.19-7.15 (m, 1H), 4.39 (s, 2H), 4.34-4.22 (m, 2H), 4.13 (s, 2H), 4.02-3.87 (m, 2H), 3.85-3.66 (m, 5H), 3.61-3.44 (m, 7H), 3.37 (s, 4H), 3.30-3.05 (m, 8H), 2.96 (s, 3H), 2.78-2.72 (m, 2H), 2.52-2.27 (m, 4H), 2.22-2.03 (m, 3H), 1.85-1.62 (m, 3H), 1.31-1.27 (m, 3H).

### Step 2: Synthesis of I-918 and I-917

I-241 (10 mg, 10.04 μmol, 1 eq) was purified by SFC separation (column: DAICEL CHIRALPAK IK (250 mm*50 mm, 10 um); mobile phase: [Hexane-EtOH (0.1% NH₃·H₂O)]; B%: 70%, isocratic elution mode) to afford Peak 1 and Peak 2. Peak 1 was purified by prep-HPLC (column: Phenomenex luna C18 150*25 mm*10 um; mobile phase: [water (TFA)-ACN]; gradient: 12%-42% B over 15 min), then concentrated to remove organic solvents and lyophilized to give I-918 (2.50 mg, 2.48 μmol, 24.76% yield, 99.02% purity) as an off-white solid. LCMS (Method E): Rt=0.423 min, [M+H]⁺=996.5. SFC: Rt=5.484 min, ee value=82.8%. ¹H NMR (400 MHz, METHANOL-d₄) δ=8.70 (d, J=1.6 Hz, 1H), 8.40-8.31 (m, 2H), 7.99-7.92 (m, 1H), 7.90-7.83 (m, 2H), 7.54-7.49 (m, 3H), 7.47-7.42 (m, 1H), 7.38-7.33 (m, 2H), 7.21-7.15 (m, 1H), 4.39 (s, 2H), 4.30-4.26 (m, 1H), 4.21 (d, J=8.4 Hz, 1H), 4.07 (s, 2H), 3.83-3.67 (m, 7H), 3.57-3.48 (m, 4H), 3.17-3.11 (m, 3H), 3.07-3.01 (m, 3H), 2.96 (s, 4H), 2.79-2.68 (m, 5H), 2.48-2.35 (m, 3H), 2.17-2.02 (m, 6H), 1.71-1.58 (m, 3H), 1.32-1.27 (m, 6H).

Peak 2 was purified by prep-HPLC (column: Phenomenex luna C18 150*25 mm*10 um; mobile phase: [water (TFA)-ACN]; gradient: 12%-42% B over 15 min), then concentrated to remove organic solvents and lyophilized to give I-917 (2.43 mg, 2.36 μmol, 23.51% yield, 96.76% purity) as an off-white solid. LCMS (Method E): Rt=0.417 min, [M+H]⁺=996.5. SFC: Rt=6.056 min, ee value=82.26%. ¹H NMR (400 MHz, METHANOL-d₄) δ=8.74-8.67 (m, 1H), 8.40-8.35 (m, 1H), 8.31 (s, 1H), 7.99-7.92 (m, 1H), 7.91-7.81 (m, 2H), 7.55-7.48 (m, 3H), 7.47-7.43 (m, 1H), 7.40-7.32 (m, 2H), 7.20-7.15 (m, 1H), 4.39 (s, 2H), 4.30-4.19 (m, 2H), 4.08 (s, 2H), 3.84-3.68 (m, 6H), 3.57-3.49 (m, 3H), 3.36 (d, J=3.6 Hz, 2H), 3.25-3.19 (m, 2H), 3.16-3.00 (m, 7H), 2.96 (s, 3H), 2.83-2.69 (m, 4H), 2.48-2.34 (m, 3H), 2.24-2.00 (m, 5H), 1.73-1.58 (m, 3H), 1.34-1.27 (m, 6H).

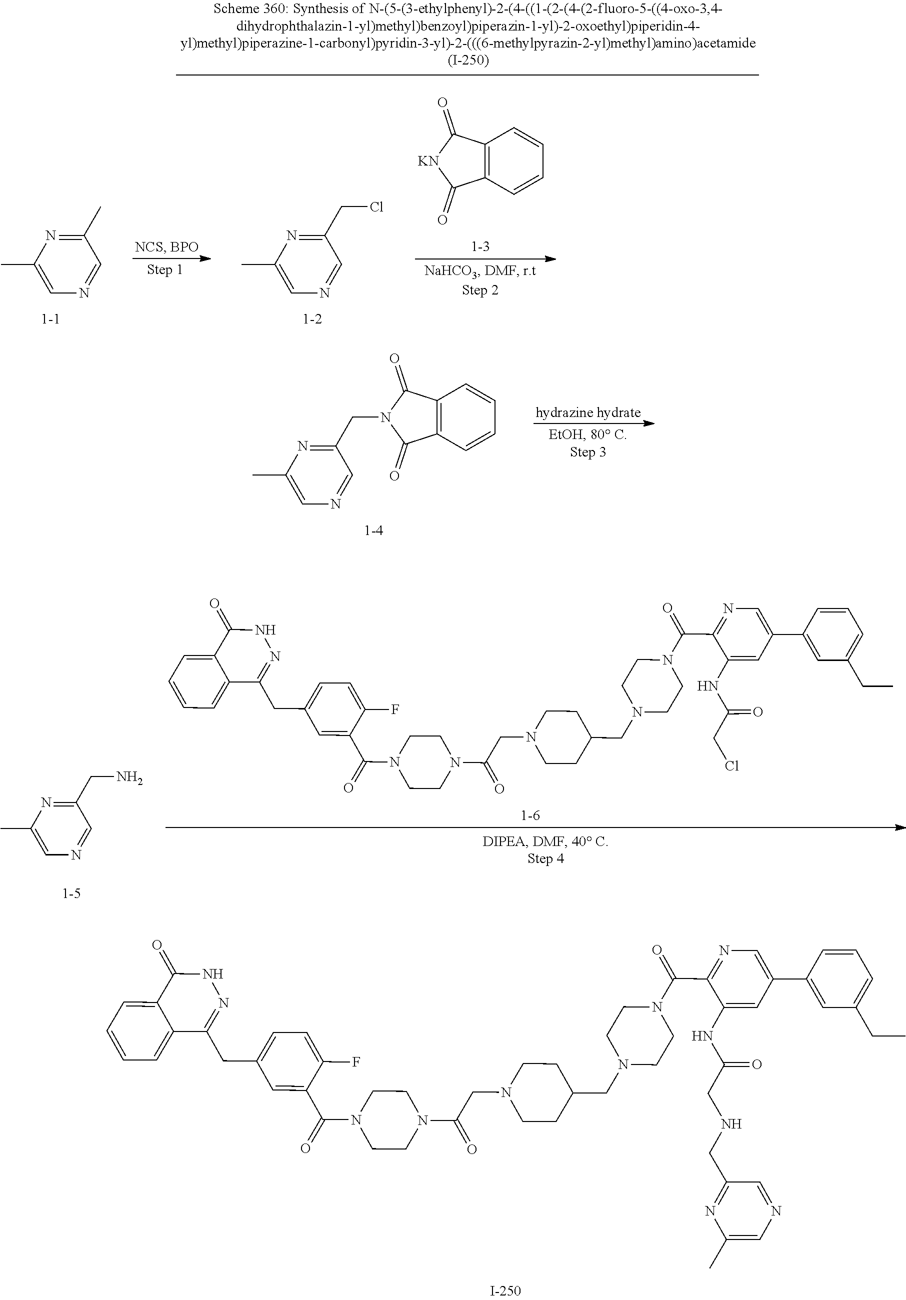

### Step 1 Synthesis of Intermediate 1-2

A mixture of Intermediate 1-1 (1.5 g 13.87 mmol, 1 eq) and NCS (2.04 g, 15.26 mmol, 1.1 eq) in CCl₄ (30 mL) was degassed and purged with N₂ 3 times, and then the mixture was stirred at 85° C., BPO (167.99 mg, 693.54 μmol, 0.05 eq) was added to the above mixture at 85° C., then the mixture stirred for 18 hr under N₂ atmosphere. The reaction was diluted with water (20 mL) and extracted with dichloromethane. The combined organic layers were dried over sodium sulfate, filtered and concentrated in vacuo. The residue was purified by flash silica gel chromatography (ISCO; 20 g SepaFlash Silica Flash Column, Eluent of 0˜100% Ethyl acetate/Petroleum ether gradient @80 mL/min). Rf=0.23 (PE:EA=1:1). Intermediate 1-2 (770 mg, 4.78 mmol, 34.46% yield, 88.5% purity) was obtained as colorless oil. LCMS (Method D): Rt=0.211 min, [M+H]⁺=143.2.

### Step 2 Synthesis of Intermediate 1-4

To a solution of Intermediate 1-2 (770 mg, 5.40 mmol, 1 eq) in DMF (5 mL) was added NaHCO₃ (680.49 mg, 8.10 mmol, 315.19 μL, 1.5 eq) and Intermediate 1-3 (1.00 g, 5.40 mmol, 1 eq). The mixture was stirred at 25° C. for 16 hr. The reaction mixture was partitioned between H₂O (20 mL) and EA (20 mL). The organic phase was separated, washed with saturated NaCl (20 mL), dried over Na₂SO₄, filtered and concentrated under reduced pressure to give a residue. The residue was purified by flash silica gel chromatography (ISCO; 20 g SepaFlash Silica Flash Column, Eluent of 0˜100% Ethyl acetate/Petroleum ether gradient @80 mL/min). Rf=0.20 (PE:EA=1:1). Intermediate 1-4 (550 mg, 1.95 mmol, 36.07% yield, 89.7% purity) was obtained as a white solid. LCMS (Method D): Rt=0.325 min, [M+H]=254.1.

### Step 3: Synthesis of Intermediate 1-5

A mixture of Intermediate 1-4 (200 mg, 789.72 μmol, 1 eq), NH₂NH₂·H₂O (310 mg, 6.19 mmol, 300.39 μL, 7.84 eq) in EtOH (2 mL) was degassed and purged with N₂ 3 times, and then the mixture was stirred at 70° C. for 1 hr under N₂ atmosphere. The resulting mixture was filtered, and the filtrate was concentrated under reduced pressure to give Intermediate 1-5 (50 mg, crude) as a white solid. LCMS: Rt=0.431 min, [M+H]⁺=124.0

### Step 4: Synthesis of I-250

To a solution of Intermediate 1-5 (8.30 mg, 67.38 μmol, 1.2 eq) in DMF (3 mL) was added DIPEA (14.51 mg, 112.30 μmol, 19.56 μL, 2 eq) and Intermediate 1-6 (50 mg, 56.15 μmol, 1 eq). The mixture was stirred at 40° C. for 16 hr. The mixture was concentrated under reduced pressure to give a residue. The residue was purified by prep-HPLC (TFA condition). The eluent was concentrated under reduced pressure to remove ACN and lyophilized to give I-250 (7.04 mg, 6.45 μmol, 11.49% yield, TFA salt) as a white solid. LCMS (Method F): Rt=0.628 min, [M+H]⁺=977.6. ¹H NMR (400 MHz, METHANOL-d₄) δ=8.79 (s, 1H), 8.65 (s, 1H), 8.60 (d, J=1.9 Hz, 1H), 8.42-8.33 (m, 2H), 7.97-7.92 (m, 1H), 7.91-7.80 (m, 2H), 7.55-7.47 (m, 3H), 7.45-7.40 (m, 1H), 7.39-7.35 (m, 1H), 7.34-7.29 (m, 1H), 7.20-7.11 (m, 1H), 4.57 (br s, 1H), 4.38 (s, 2H), 4.00 (s, 2H), 3.80-3.73 (m, 4H), 3.72-3.66 (m, 2H), 3.55-3.50 (m, 5H), 3.37-3.32 (m, 2H), 3.29-3.22 (m, 2H), 3.00-2.90 (m, 2H), 2.78-2.71 (m, 2H), 2.55 (s, 3H), 2.45 (br s, 4H), 2.25-2.19 (m, 2H), 2.18-2.08 (m, 2H), 1.82-1.74 (m, 2H), 1.64-1.52 (m, 1H), 1.31-1.22 (m, 5H). ¹⁹F NMR (377 MHz, METHANOL-d₄) δ=−76.948, −120.778.

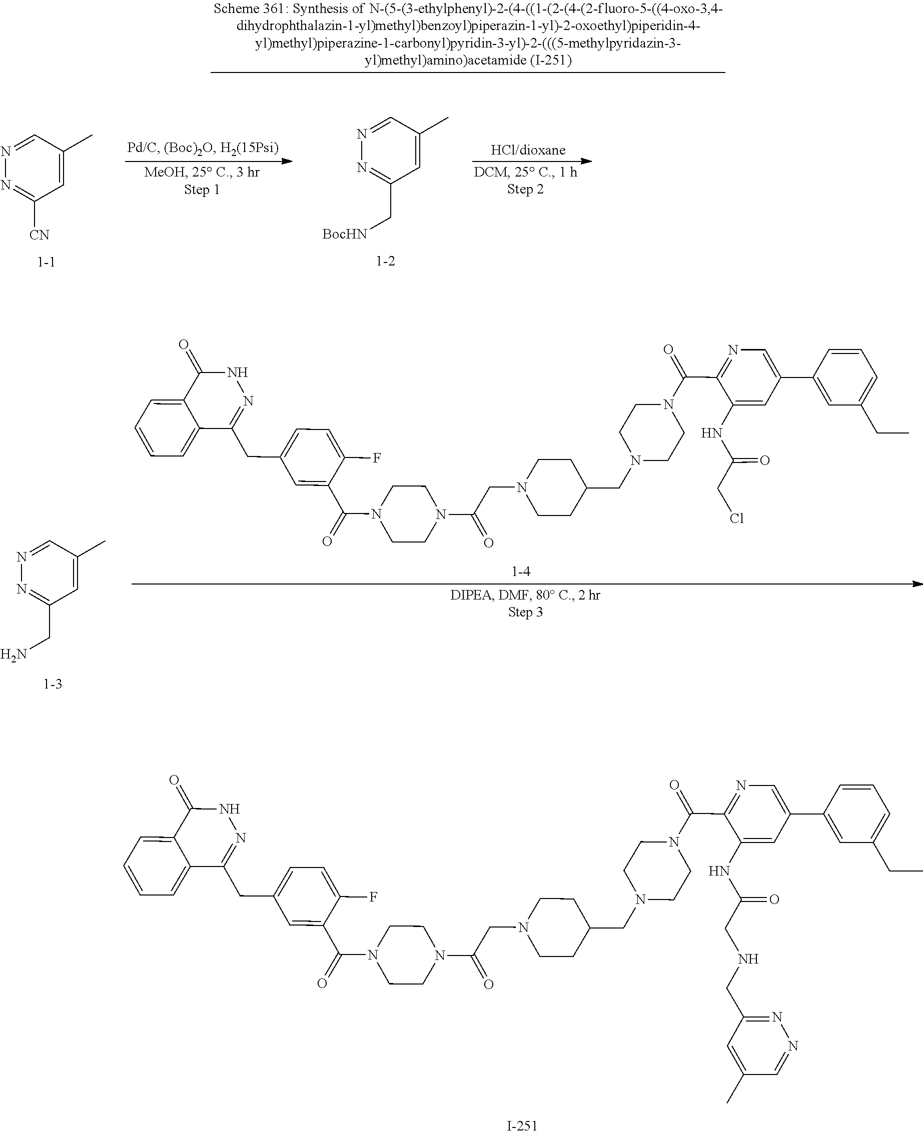

### Step 1: Synthesis of Intermediate 1-2

To a solution of Pd/C (100 mg, 93.97 μmol, 10% purity, 5.60e−2 eq) in MeOH (2 mL) was added Intermediate 1-1 (200 mg, 1.68 mmol, 1 eq) and (Boc)₂O (732.85 mg, 3.36 mmol, 771.42 μL, 2 eq) under N₂ atmosphere. The suspension was degassed and purged with H₂ 3 times. The mixture was stirred under H₂ (15 Psi) at 25° C. for 3 hr. The reaction mixture was filtered and washed with MeOH 4 mL (2 mL*2). The filtrate was concentrated under reduced pressure to give a residue. The residue was purified by prep-TLC (SiO₂, DCM:MeOH=10:1, Rf=0.30, UV=254 nm). The eluent was concentrated under reduced pressure to give Intermediate 1-2 (110 mg, 477.40 μmol, 28.43% yield, 96.9% purity) was obtained as yellow oil. LCMS (Method C): Rt=0.555 min, [M+H]⁺=223.9.

### Step 2: Synthesis of Intermediate 1-3

A solution of Intermediate 1-2 (110 mg, 492.68 μmol, 1 eq) in CH₂Cl₂ (0.5 mL) and HCl/dioxane (2 M, 611.11 μL, 2.48 eq) was stirred at 25° C. for 1 hr. The mixture was concentrated under reduced pressure to give Intermediate 1-3 (80 mg, crude, HCl salt) as a white solid and was used in the next step without further purification. LCMS (Method C): Rt=0.340 min, [M+H]⁺=124.0. ¹H NMR (400 MHz, METHANOL-d₄) δ=9.54 (s, 1H), 8.32-8.24 (m, 1H), 4.63 (s, 2H), 2.65 (s, 3H)

### Step 3: Synthesis of I-251

To a solution of Intermediate 1-3 (12.91 mg, 80.86 μmol, 1.2 eq, HCl salt) in DMF (0.5 mL) was added Intermediate 1-4 (60 mg, 67.38 μmol, 1 eq) and DIPEA (26.13 mg, 202.15 μmol, 35.21 μL, 3 eq). The mixture was stirred at 80° C. for 2 hr. The mixture was filtered. The filtrate was purified by reversed-phase HPLC (0.1% FA condition). The eluent was concentrated under reduced pressure to remove ACN and then lyophilized to give I-251 (44.26 mg, 40.75 μmol, 60.47% yield, 94.2% purity, FA salt) as an orange gum. LCMS (Method D): Rt=0.329 min, [M+H]⁺=977.3. ¹H NMR (400 MHz, METHANOL-d₄) δ=8.97 (s, 1H), 8.72 (s, 1H), 8.61 (s, 1H), 8.36 (d, J=7.6 Hz, 1H), 8.33 (s, 1H), 7.97-7.92 (m, 1H), 7.90-7.78 (m, 3H), 7.55-7.47 (m, 3H), 7.46-7.40 (m, 1H), 7.39-7.29 (m, 2H), 7.21-7.11 (m, 1H), 4.38 (s, 2H), 4.14 (s, 2H), 4.08 (s, 1H), 3.99 (s, 1H), 3.86-3.65 (m, 6H), 3.54 (s, 6H), 3.50-3.42 (m, 2H), 3.38 (s, 2H), 2.96-2.82 (m, 2H), 2.79-2.69 (m, 2H), 2.52 (s, 4H), 2.43 (s, 3H), 2.37-2.27 (m, 2H), 2.07-1.94 (m, 2H), 1.93-1.78 (m, 1H), 1.57-1.42 (m, 2H), 1.34-1.24 (m, 3H). ¹⁹F NMR (376 MHz, METHANOL-d₄) δ=−120.700.

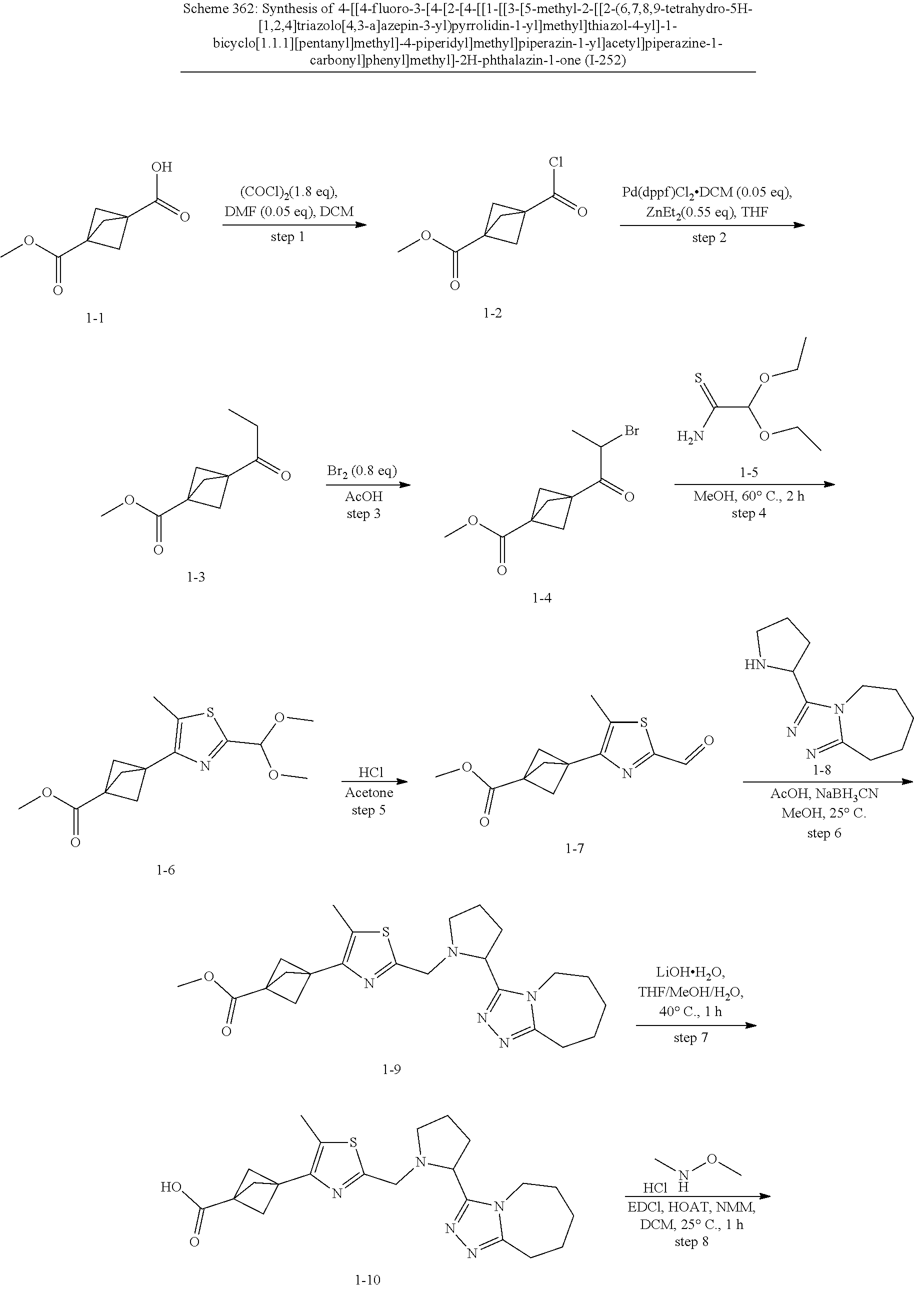

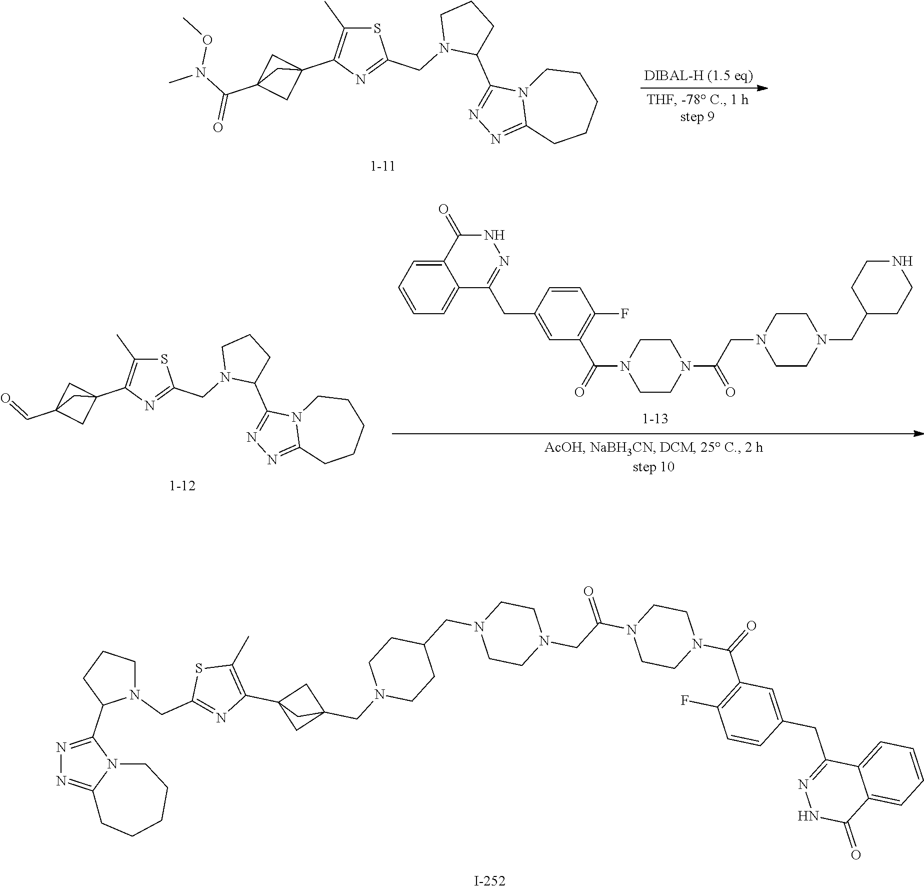 [SMILES: CON(C)C(=O)C12CC(c3nc(CN4CCCC4c4nnc5n4CCCCC5)sc3C)(C1)C2 ; Cc1sc(CN2CCCC2c2nnc3n2CCCCC3)nc1C12CC(C=O)(C1)C2 ; O=C(CN1CCN(CC2CCNCC2)CC1)N1CCN(C(=O)c2cc(Cc3n[nH]c(=O)c4ccccc34)ccc2F)CC1 ; Cc1sc(CN2CCCC2c2nnc3n2CCCCC3)nc1C12CC(CN3CCC(CN4CCN(CC(=O)N5CCN(C(=O)c6cc(Cc7n[nH]c(=O)c8ccccc78)ccc6F)CC5)CC4)CC3)(C1)C2]

### Step 1: Synthesis of Intermediate 1-2

To a solution of Intermediate 1-1 (19 g, 111.66 mmol, 1 eq) in DCM (190 mL) was added DMF (408.08 mg, 5.58 mmol, 429.55 μL, 0.05 eq). Then (COCl)₂ (25.51 g, 200.98 mmol, 17.59 mL, 1.8 eq) was dropwise added into the mixture at 0° C. and the mixture was stirred at 25° C. for 2 hr. The mixture was concentrated to give Intermediate 1-2 (19 g, 100.74 mmol, 90.22% yield) as a yellow solid. LCMS (Method D): Rt: 0.262 min, [M+H-35.5+31]⁺=185.0.

### Step 2: Synthesis of Intermediate 1-3

To a solution of Intermediate 1-2 (19 g, 100.74 mmol, 1 eq) in THF (190 mL) was added Pd(dppf)Cl₂·CH₂Cl₂ (4.11 g, 5.04 mmol, 0.05 eq). The reaction mixture was degassed with N₂ three times. Then ZnEt₂ (1 M, 55.41 mL, 0.55 eq) was dropwise added into the mixture at 0° C. and the mixture was stirred at 0° C. for 2 hr under N₂. The reaction mixture was dropwise added into 1M HCl aqueous (100 mL), then extracted by EA (50 mL×2). The combined organic layer was washed with brine (50 mL), dried over Na₂SO₄, filtered and concentrated to give residue. The residue was purified by flash silica gel chromatography (ISCO; 330 g SepaFlash Silica Flash Column, Eluent of 15˜40% Ethyl acetate/Petroleum ether gradient @150 mL/min). The eluent was concentrated under reduced pressure to give Intermediate 1-3 (9.4 g, 35.67 mmol, 35.41% yield) as colorless oil. ¹H NMR (400 MHz, CHLOROFORM-d) δ=3.71-3.70 (m, 3H), 2.40-2.45 (m, 2H), 2.31-2.28 (m, 6H), 1.05 (t, J=7.6 Hz, 3H).

### Step 3: Synthesis of Intermediate 1-4

To a mixture of Intermediate 1-3 (8 g, 43.90 mmol, 1 eq) in AcOH (70 mL) was added Br₂ (5.61 g, 35.12 mmol, 1.81 mL, 0.8 eq) at 20° C. and then the mixture was stirred at 20° C. for 2 hr under N₂ atmosphere. The reaction mixture was quenched by addition of H₂O (50 mL) at 10° C., and then the reaction mixture was diluted with and extracted with EA (20 mL*3). The combined organic layers were washed with saturated solution of NaHCO₃ (20 mL*3), dried over Na₂SO₄, filtered and concentrated under reduced pressure to give a residue. The residue was purified by flash silica gel chromatography (ISCO; 120 g SepaFlash Silica Flash Column, Eluent of 0˜20% Ethyl acetate/Petroleum ether gradient @80 mL/min). The eluent was concentrated to give Intermediate 1-4 (8 g, 30.64 mmol, 69.78% yield) as yellow oil. ¹H NMR (400 MHz, CHLOROFORM-d) δ=4.54 (q, J=6.8 Hz, 1H), 3.71 (s, 3H), 2.45-2.38 (m, 6H), 1.73 (d, J=6.8 Hz, 3H).

### Step 4: Synthesis of Intermediate 1-6

To a solution of Intermediate 1-4 (4.38 g, 26.81 mmol, 1 eq) in MeOH (70 mL) was added Intermediate 1-5 (7 g, 26.81 mmol, 1 eq). The mixture was stirred at 60° C. for 2 hr. The mixture was concentrated to give a residue. The residue was purified by flash silica gel chromatography (ISCO; 80 g SepaFlash Silica Flash Column, Eluent of 0˜20% Ethyl acetate/Petroleum ether gradient @80 mL/min). The eluent was concentrated under reduced pressure to give Intermediate 1-6 (2.9 g, crude) as brown oil. LCMS (Method D): Rt: 0.395 min, [M+H]⁺=298.0.

### Step 5: Synthesis of Intermediate 1-7

To a solution of Intermediate 1-6 (200 mg, 672.56 μmol, 1 eq) in acetone (16 mL) was added HCl (1 M, 1.60 mL, 2.38 eq). The mixture was stirred at 40° C. for 10 hr. The mixture was adjusted to pH=7-8 with an aqueous solution of sodium bicarbonate. The reaction mixture was quenched with water (210 mL) and extracted with ethyl acetate (70 mL*3). The combined organic layers were dried over sodium sulfate, filtered and concentrated under reduced pressure to give Intermediate 1-7 (1.2 g, crude) as a brown solid. LCMS (Method D): Rt: 0.359 min, [M+H]⁺=252.0. ¹H NMR (400 MHz, CHLOROFORM-d) δ=9.85 (s, 1H), 3.73 (s, 3H), 2.56 (s, 3H), 2.53 (s, 6H).

### Step 6: Synthesis of Intermediate 1-9

To a solution of Intermediate 1-7 (1.2 g, 4.78 mmol, 1 eq) and Intermediate 1-8 (3.48 g, 14.33 mmol, 3 eq, HCl salt) in MeOH (20 mL) was added AcOH (286.76 mg, 4.78 mmol, 273.36 μL, 1 eq). The mixture was stirred at 25° C. for 0.1 hr. Then NaBH₃CN (600.16 mg, 9.55 mmol, 2 eq) was added into the mixture and it was stirred at 25° C. for 2 hr. The mixture was concentrated to give a residue. Then the reaction mixture was diluted with water (60 mL) and extracted with ethyl acetate (20 mL×3). The combined organic layers were washed with brine (20 mL×3) and dried over sodium sulfate, filtered and concentrated under reduced pressure to give a residue. The residue was purified by flash silica gel chromatography (ISCO; 40 g SepaFlash Silica Flash Column, Eluent of 0˜50% Ethyl acetate/Methanol gradient @60 mL/min). The eluent was concentrated under reduced pressure to give Intermediate 1-9 (900 mg, 1.61 mmol, 33.71% yield, 78.99% purity) as a brown gum. LCMS (Method D): Rt: 0.317 min, [M+H]⁺=442.2. ¹H NMR (400 MHz, METHANOL-d₄) δ=4.45-4.24 (m, 2H), 4.04-3.96 (m, 1H), 3.87 (d, J=14.8 Hz, 1H), 3.78-3.66 (m, 4H), 3.27-3.19 (m, 1H), 3.07-2.85 (m, 2H), 2.62-2.13 (m, 10H), 2.06-1.87 (m, 5H), 1.85-1.61 (m, 4H).

### Step 7: Synthesis of Intermediate 1-10

To a solution of Intermediate 1-9 (300 mg, 679.37 μmol, 1 eq) in THF (1.5 mL), H₂O (0.3 mL) and MeOH (1.5 mL) was added LiOH·H₂O (85.53 mg, 2.04 mmol, 3 eq). The mixture was stirred at 40° C. for 1 hr. The mixture was concentrated to give a residue. The reaction mixture was diluted with water (10 mL). Then the mixture was adjusted to pH=6-7 with a saturated citric acid aqueous solution. Then the reaction mixture was extracted with DCM (4 mL×3). The combined organic layers were washed with brine (4 mL×3) and dried over sodium sulfate, filtered and concentrated under reduced pressure to give Intermediate 1-10 (240 mg, crude) as a yellow gum. LCMS (Method D): Rt: 0281 min, [M+H]⁺=428.1.

### Step 8: Synthesis of Intermediate 1-11

To a solution of Intermediate 1-10 (240 mg, 561.32 μmol, 1 eq) and N-methoxymethanamine;hydrochloride (54.75 mg, 561.32 μmol, 1 eq) in DCM (2 mL) was added EDCI (322.82 mg, 1.68 mmol, 3 eq), NMM (283.88 mg, 2.81 mmol, 308.57 μL, 5 eq) and HOAt (76.40 mg, 561.32 μmol, 78.52 μL, 1 eq). The mixture was stirred at 25° C. for 1 hr. The reaction mixture was diluted with water (9 mL) and extracted with DCM (3 mL×3). The combined organic layers were washed with brine (3 mL×3) and dried over sodium sulfate, filtered and concentrated under reduced pressure to give Intermediate 1-11 (400 mg, crude) as yellow oil. LCMS (Method D): Rt: 0.310 min, [M+H]⁺=471.4. ¹H NMR (400 MHz, CHLOROFORM-d) δ=4.44-4.21 (m, 2H), 4.11-4.08 (m, 1H), 3.93 (d, J=15.2 Hz, 1H), 3.77 (s, 1H), 3.72-3.69 (m, 3H), 3.65 (d, J=15.2 Hz, 1H), 3.34-3.29 (m, 1H), 3.21 (s, 3H), 3.00-2.94 (m, 2H), 2.67-2.66 (m, 1H), 2.51 (s, 7H), 2.40 (s, 3H), 2.03-1.88 (m, 6H), 1.79 (s, 2H).

### Step 9: Synthesis of Intermediate 1-12

To a solution of Intermediate 1-11 (400 mg, 849.93 μmol, 1 eq) in THF (4 mL) was added DIBAL-H (1 M, 1.27 mL, 1.5 eq) at −78° C. The mixture was stirred at −78° C. for 1 hr. The reaction mixture was quenched with a saturated ammonium chloride solution (9 mL) at −78° C., and then extracted with EA (3 mL*3). The combined organic layers were dried over Na₂SO₄, filtered and concentrated under reduced pressure to give Intermediate 1-12 (220 mg, crude) as a yellow solid. LCMS (Method D): Rt: 0.264 min, [M+H]⁺=412.0. ¹H NMR (400 MHz, DMSO-d₆) δ=9.58 (s, 1H), 4.33-4.08 (m, 2H), 3.99-3.89 (m, 1H), 3.80-3.68 (m, 1H), 3.60-3.53 (m, 1H), 3.38 (s, 1H), 3.13 (d, J=9.2 Hz, 1H), 2.93-2.76 (m, 2H), 2.37-2.27 (m, 6H), 2.05-1.92 (m, 4H), 1.92-1.82 (m, 3H), 1.77 (s, 2H), 1.67 (s, 2H), 1.56 (d, J=5.2 Hz, 2H).

### Step 10: Synthesis of I-252

To a solution of Intermediate 1-12 (40 mg, 97.19 μmol, 1 eq) and Intermediate 1-13 (60.86 mg, 97.19 μmol, 1 eq, HCl salt) in DCM (1 mL) was added AcOH (5.84 mg, 97.19 μmol, 5.56 μL, 1 eq). The mixture was stirred at 25° C. for 0.5 hr. Then NaBH₃CN (12.22 mg, 194.38 μmol, 2 eq) was added into the mixture and it was stirred at 25° C. for 1.5 hr. The reaction mixture was diluted with water (6 mL) and extracted with DCM (2 mL×3). The combined organic layers were washed with brine (2 mL×3) and dried over sodium sulfate, filtered and concentrated under reduced pressure to give a residue. The residue was purified by prep-HPLC (column: CD01-Phenomenex luna C18 150*25*10 um; mobile phase: [water (FA)-ACN]; gradient: 10%-40% B over 12 min). The eluent was concentrated under reduced pressure to remove ACN and then lyophilized to give I-252 (4.18 mg, 4.24 μmol, 4.37% yield, 100% purity, FA salt) as a white solid. LCMS (Method D): Rt: 0.288 min, [M+H]⁺=985.4. ¹H NMR (400 MHz, METHANOL-d₄) δ=8.43-8.35 (m, 2H), 7.99-7.92 (m, 1H), 7.92-7.79 (m, 2H), 7.50 (d, J=2.8 Hz, 1H), 7.37-7.35 (m, 1H), 7.19-7.17 (m, 1H), 4.43-4.29 (m, 4H), 4.00-3.98 (m, 1H), 3.91-3.83 (m, 1H), 3.80 (s, 1H), 3.77-3.65 (m, 4H), 3.60-3.48 (m, 4H), 3.43 (s, 1H), 3.36 (s, 2H), 3.29 (s, 3H), 3.03-2.90 (m, 4H), 2.77-2.54 (m, 8H), 2.53-2.12 (m, 13H), 2.10-1.86 (m, 9H), 1.82-1.65 (m, 4H), 1.49 (d, J=11.2 Hz, 2H). ¹⁹F NMR (377 MHz, METHAN OL-d₄) δ=−120.794. SFC: Rt: 2.736, 3.510 min.

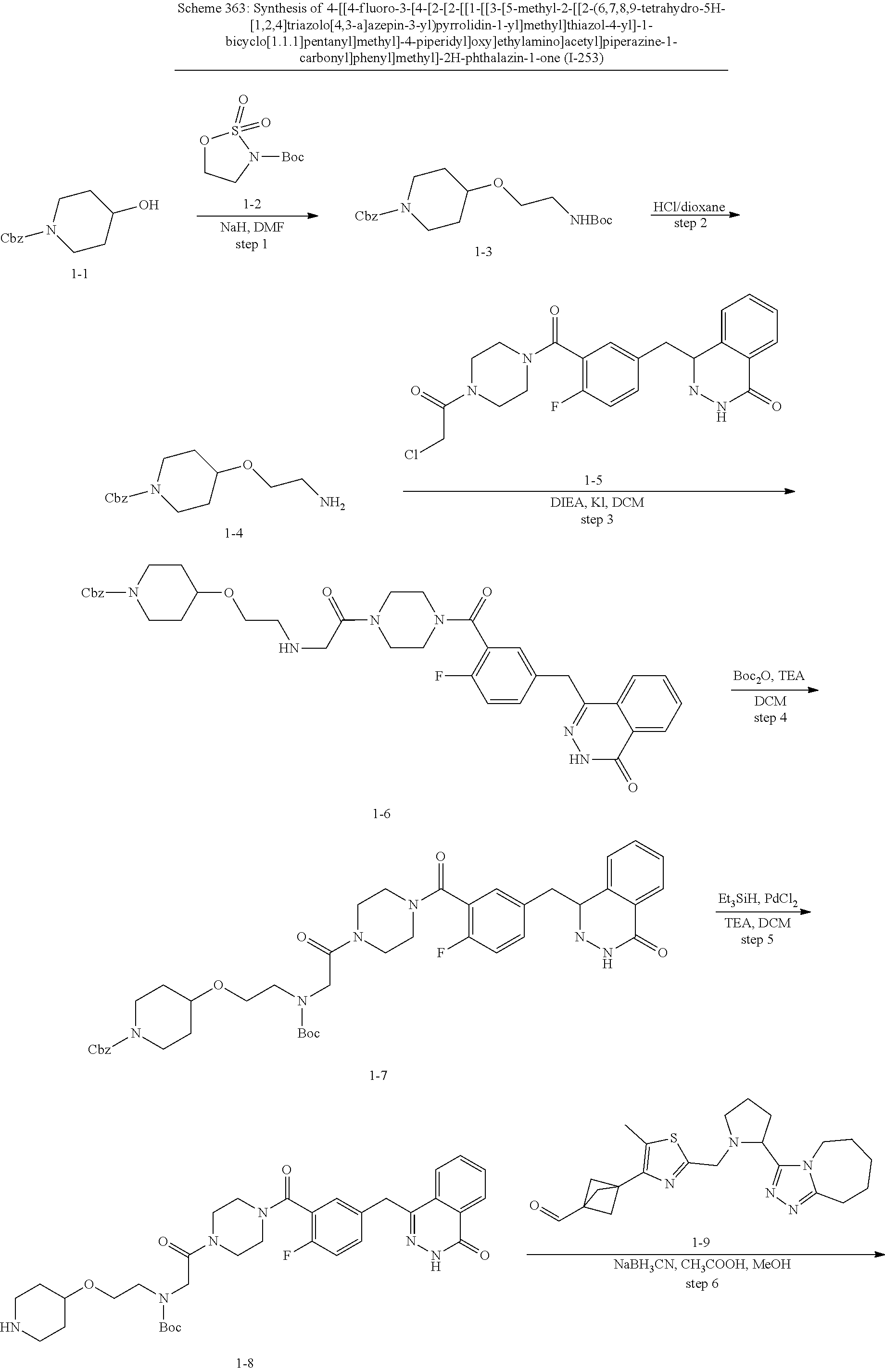

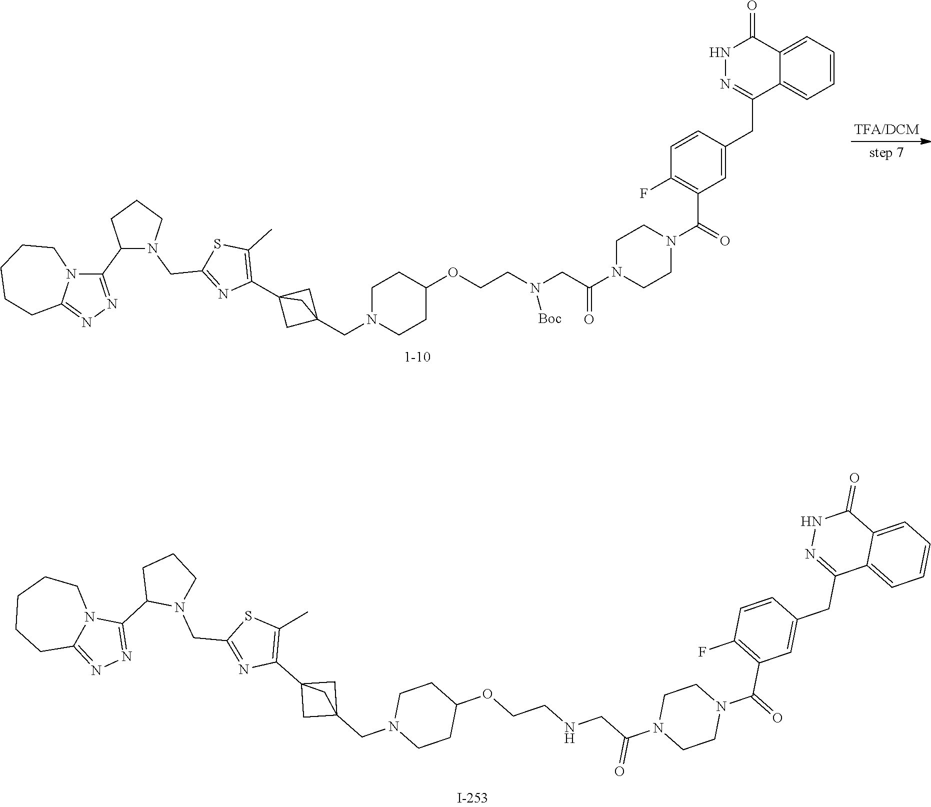 [SMILES: Cc1sc(CN2CCCC2c2nnc3n2CCCCC3)nc1C12CC(CN3CCC(OCCN(CC(=O)N4CCN(C(=O)c5cc(Cc6n[nH]c(=O)c7ccccc67)ccc5F)CC4)C(=O)OC(C)(C)C)CC3)(C1)C2 ; Cc1sc(CN2CCCC2c2nnc3n2CCCCC3)nc1C12CC(CN3CCC(OCCNCC(=O)N4CCN(C(=O)c5cc(Cc6n[nH]c(=O)c7ccccc67)ccc5F)CC4)CC3)(C1)C2]

### Step 1: Synthesis of Intermediate 1-3

To a 250 ml three-neck round-bottomed flask was added NaH (1.02 g, 25.50 mmol, 60% purity, 1.2 eq) and DMF (50 mL). The suspension was degassed and purged with N₂ 3 times. Then Intermediate 1-1 (5 g, 21.25 mmol, 1 eq) in DMF (15 ml) was added dropwise at 0° C. Then the mixture was stirred at 25° C. for 0.5 hr. Intermediate 1-2 (4.98 g, 22.31 mmol, 1.05 eq) was added dropwise at 0° C. and stirred at 25° C. for 2.5 hr under N₂. The mixture was quenched by aq. NH₄Cl (120 ml) and extracted with EA (70 mL*3). The combined organic layers dried over sodium sulfate, filtered and concentrated under reduced pressure to give a residue. The crude product was purified by reversed-phase HPLC (0.1% FA condition). The eluent was concentrated under reduced pressure to remove ACN and then lyophilized to give the product. Intermediate 1-3 (3.5 g, 8.91 mmol, 41.94% yield, 96.378% purity) as yellow oil. LCMS (Method D): Retention time: 0.441 min, [M+H-Boc]=279.0. ¹H NMR (400 MHz, METHANOL-d₄) δ=7.41-7.24 (m, 5H), 5.11 (s, 2H), 3.74 (d, J=4.4 Hz, 2H), 3.57-3.48 (m, 3H), 3.27 (br s, 2H), 3.20-3.18 (m 2H), 1.83-1.80 (m, 2H), 1.56-1.48 (m, 2H), 1.43 (s, 9H).

### Step 2: Synthesis of Intermediate 1-4

To a solution of Intermediate 1-3 (2 g, 5.28 mmol, 1 eq) in DCM (10 mL) was added HCl/dioxane (2 M, 10 mL). The mixture was stirred at 25° C. for 1 hr. The mixture was concentrated to give a residue. The residue was used for the next step directly. Intermediate 1-4 (1.6 g, crude, HCl salt) was obtained as a white solid. LCMS (Method D): Retention time: 0.235 min, [M+H]⁺=279.1.

### Step 3: Synthesis of Intermediate 1-6

To a solution of Intermediate 1-4 (1.5 g, 4.76 mmol, 1 eq, HCl salt) and Intermediate 1-5 (2.11 g, 4.76 mmol, 1 eq) in DCM (20 mL) was added DIEA (1.85 g, 14.29 mmol, 2.49 mL, 3 eq) and KI (79.10 mg, 476.48 μmol, 0.1 eq). The mixture was stirred at 40° C. for 1 hr. The reaction mixture was diluted with water (60 mL) and extracted with DCM (20 mL×3). The combined organic layers were washed with brine (20 mL×3) and dried over sodium sulfate, filtered and concentrated under reduced pressure to give a residue. The crude product was purified by reversed-phase HPLC (H₂O). The eluent was concentrated under reduced pressure to remove ACN and then lyophilized to give the product. Intermediate 1-6 (1.2 g, 1.69 mmol, 35.51% yield, 96.559% purity) was obtained as a brown solid. LCMS (Method D): Retention time: 0.347 min, [M+H]⁺=685.4. ¹H NMR (400 MHz, METHANOL-d₄) δ=8.37-8.34 (m, 1H), 7.98-7.89 (m, 1H), 7.84-7.81 (m, 2H), 7.51-7.43 (m, 1H), 7.37 (s, 1H), 7.35-7.33 (m, 3H), 7.32-7.27 (m, 1H), 7.15 (t, J=8.8 Hz, 1H), 5.10 (s, 2H), 4.37 (s, 2H), 3.81-3.64 (m, 5H), 3.64-3.43 (m, 7H), 3.39 (d, J=4.0 Hz, 1H), 3.35 (s, 3H), 3.29-3.22 (m, 2H), 2.78 (d, J=5.2 Hz, 2H), 1.84 (s, 2H), 1.60-1.35 (m, 2H).

### Step 4: Synthesis of Intermediate 1-7

To a solution of Intermediate 1-6 (1.1 g, 1.61 mmol, 1 eq) in DCM (20 mL) was added Boc₂O (420.71 mg, 1.93 mmol, 442.86 μL, 1.2 eq) and TEA (812.76 mg, 8.03 mmol, 1.12 mL, 5 eq). The mixture was stirred at 25° C. for 1 hr. The reaction mixture was washed with water (10 mL) and extracted with DCM (10 mL*3), the combined organic phase was dried by Na₂SO₄, concentrated under reduced pressure to give a residue. The residue was purified by flash silica gel chromatography (ISCO; 80 g SepaFlash Silica Flash Column, Eluent of 0˜50% Ethyl acetate/Petroleum ether @80 mL/min) and the eluent was concentrated to give product. Intermediate 1-7 (1.14 g, 1.35 mmol, 88.43% yield, 93.088% purity) was obtained as a white solid. LCMS (Method D): Retention time: 0.492 min, [M+H]⁺=785.3.

### Step 5: Synthesis of Intermediate 1-8

To a solution of Intermediate 1-7 (500 mg, 637.05 μmol, 1 eq) in DCM (5 mL) was added TEA (193.39 mg, 1.91 mmol, 266.01 μL, 3 eq), Et₃SiH (888.89 mg, 7.64 mmol, 1.22 mL, 12 eq), PdCl₂ (56.48 mg, 318.52 μmol, 0.5 eq), and the reaction mixture was stirred at 25° C. for 1 hr. The reaction mixture was washed with water (5 mL) and extracted with DCM (5 mL*3), the combined organic phase was dried by Na₂SO₄, and concentrated under reduced pressure to give a residue. The residue was purified by flash silica gel chromatography (ISCO; 20 g SepaFlash Silica Flash Column, Eluent of 0˜50% Methanol/Dichloromethane @40 mL/min) and the eluent was concentrated to give product. Intermediate 1-8 (420 mg, 627.98 μmol, 98.58% yield, 97.298% purity) was obtained as a white solid. LCMS (Method D): Retention time: 0.326 min, [M+H]⁺=651.2.

### Step 6: Synthesis of Intermediate 1-10

To a solution of Intermediate 1-9 (113.84 mg, 276.61 μmol, 1 eq) and Intermediate 1-8 (180 mg, 276.61 μmol, 1 eq) in MeOH (2 mL) was added CH₃COOH (16.61 mg, 276.61 μmol, 15.83 μL, 1 eq) at 25° C., and the mixture was stirred at 60° C. for 12 h. Then NaBH₃CN (52.15 mg, 829.83 μmol, 3 eq) were added at 60° C. The resulting mixture was stirred at 60° C. for 2 hr. The reaction mixture was washed with water (2 mL) and extracted with DCM (2 mL*3), the combined organic phase was dried by Na₂SO₄, and concentrated under reduced pressure to give a residue. The residue was purified by reversed-phase HPLC (0.1% of FA) and the eluent was lyophilized to give product. Intermediate 1-10 (30 mg, 26.23 μmol, 9.48% yield, 91.486% purity) was obtained as brown oil. LCMS (Method D): Retention time: 0.386 min, [M+H]⁺=1046.5.

### Step 7: Synthesis of I-253

To a solution of Intermediate 1-10 (20 mg, 19.11 μmol, 1 eq) in DCM (0.2 mL) was added TFA (153.50 mg, 1.35 mmol, 0.1 mL, 70.43 eq) and the mixture was stirred at 25° C. for 1 hr. The mixture was concentrated under reduced pressure to give a residue. The residue was purified by reversed-phase HPLC (0.1% of TFA) and the eluent was lyophilized to give product. I-253 (14 mg, 12.77 μmol, 66.83% yield, 96.743% purity, TFA salt) was obtained as a white solid. LCMS (Method D): Retention time: 0.286 min, [M+H]⁺=946.3. SFC: Retention time: 1.538 min, 1.921 min. ¹H NMR (400 MHz, METHANOL-d₄) δ=8.37 (d, J=7.2 Hz, 1H), 7.99-7.92 (m, 1H), 7.91-7.78 (m, 2H), 7.56-7.46 (m, 1H), 7.38 (t, J=5.2 Hz, 1H), 7.17 (t, J=9.2 Hz, 1H), 4.47-4.25 (m, 5H), 4.24-4.07 (m, 3H), 4.06-3.97 (m, 1H), 3.90-3.62 (m, 7H), 3.60-3.42 (m, 4H), 3.41-3.33 (m, 7H), 3.20-3.00 (m, 3H), 2.83-2.70 (m, 1H), 2.50-2.40 (m, 4H), 2.35 (s, 6H), 2.29-2.13 (m, 2H), 2.11-1.91 (m, 7H), 1.90-1.69 (m, 5H). ¹⁹F NMR (377 MHz, METHANOL-d₄) δ=−77.069, −120.672.

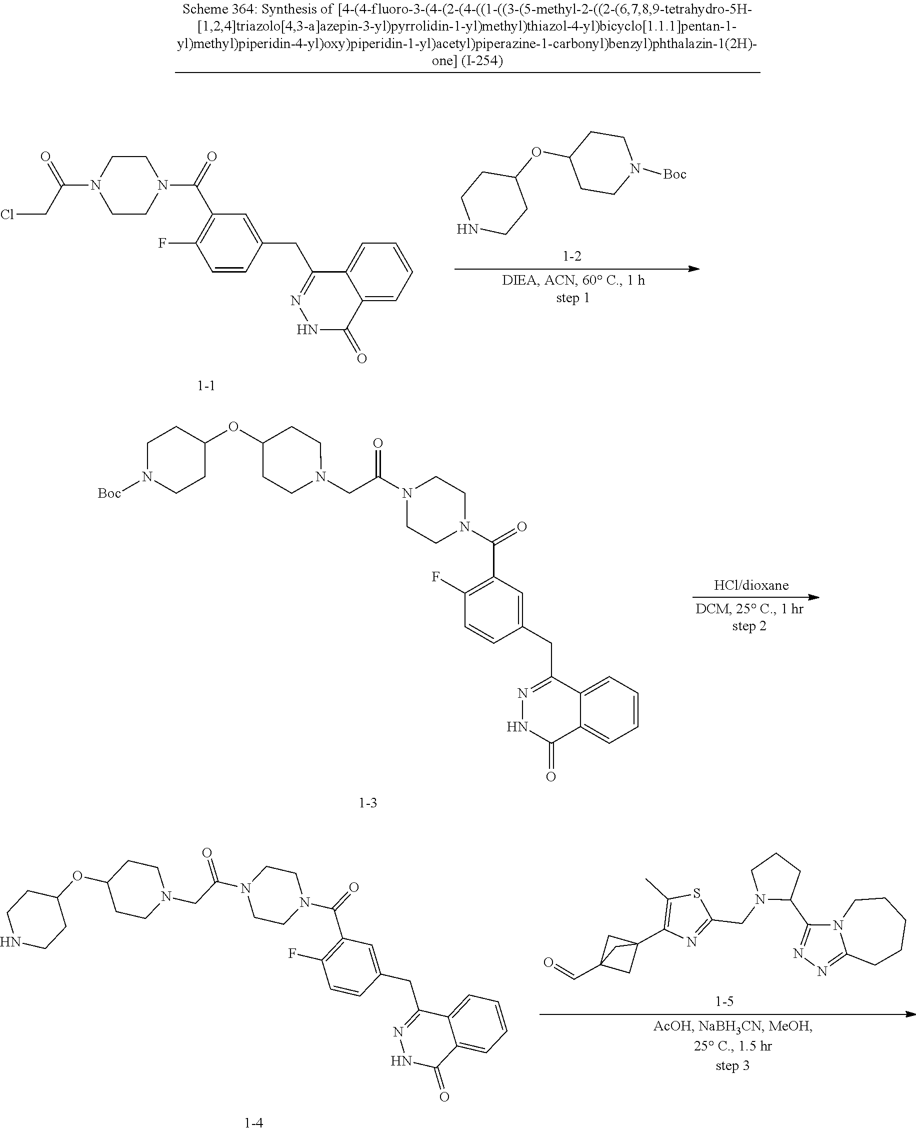 [SMILES: O=C(CCl)N1CCN(C(=O)c2cc(Cc3n[nH]c(=O)c4ccccc34)ccc2F)CC1 ; CC(C)(C)OC(=O)N1CCC(OC2CCNCC2)CC1 ; CC(C)(C)OC(=O)N1CCC(OC2CCN(CC(=O)N3CCN(C(=O)c4cc(Cc5n[nH]c(=O)c6ccccc56)ccc4F)CC3)CC2)CC1 ; O=C(CN1CCC(OC2CCNCC2)CC1)N1CCN(C(=O)c2cc(Cc3n[nH]c(=O)c4ccccc34)ccc2F)CC1 ; Cc1sc(CN2CCCC2c2nnc3n2CCCCC3)nc1C12CC(C=O)(C1)C2]

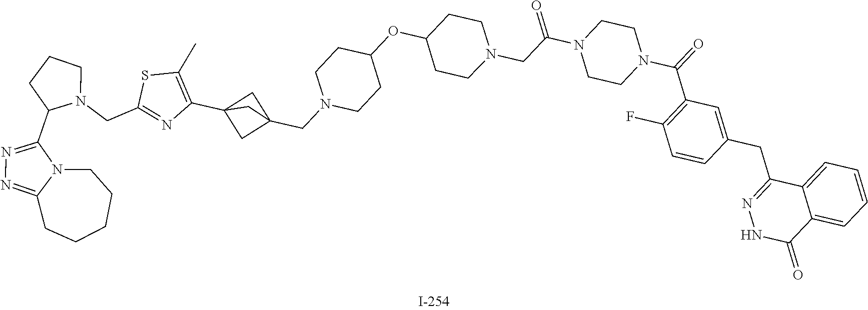 [SMILES: Cc1sc(CN2CCCC2c2nnc3n2CCCCC3)nc1C12CC(CN3CCC(OC4CCN(CC(=O)N5CCN(C(=O)c6cc(Cc7n[nH]c(=O)c8ccccc78)ccc6F)CC5)CC4)CC3)(C1)C2]

### Step 1: Synthesis of Intermediate 1-3

To a solution of Intermediate 1-2 (300 mg, 1.05 mmol, 1 eq) and Intermediate 1-1 (467.17 mg, 1.05 mmol, 1 eq) in ACN (3 mL) was added DIEA (409.00 mg, 3.16 mmol, 551.22 μL, 3 eq). The mixture was stirred at 60° C. for 1 hr. The reaction mixture was concentrated under reduced pressure to give a residue. The residue was purified by reverse phase HPLC (FA condition), the eluent was concentrated and lyophilized to give a product. Intermediate 1-3 (500 mg, 631.21 μmol, 59.84% yield, 93.019% purity, FA salt) was obtained as a white solid. LCMS (Method D): Retention time: 0.347 min, [M+H]⁺=691.5. ¹H NMR (400 MHz, METHANOL-d₄) δ=8.42-8.39 (m, 1H), 8.00-7.94 (m, 1H), 7.93-7.83 (m, 2H), 7.53 (d, J=3.2 Hz, 1H), 7.45-7.32 (m, 1H), 7.23-7.16 (m, 1H), 4.41 (s, 2H), 3.94 (s, 1H), 3.83 (s, 2H), 3.79-3.65 (m, 6H), 3.63-3.53 (m, 2H), 3.45 (s, 1H), 3.41-3.34 (m, 2H), 3.26-3.09 (m, 4H), 3.07-2.80 (m, 2H), 2.09-1.96 (m, 2H), 1.89-1.74 (m, 4H), 1.55-1.49 (m, 2H), 1.47 (s, 9H). ¹⁹F NMR (377 MHz, METHANOL-d₄) δ=−120.764.

### Step 2: Synthesis of Intermediate 1-4

To a solution of Intermediate 1-3 (200 mg, 271.43 μmol, 1 eq, FA salt) in DCM (2 mL) was added HCl/dioxane (2 M, 2 mL). The mixture was stirred at 25° C. for 1 hr. The reaction mixture was concentrated under reduced pressure to give a residue. The residue was dissolved in MeOH (5 mL), then ion-exchange resin (500 mg) was added, and the mixture was stirred at 25° C. for 1 hr until the pH reached 8-9. Then the mixture was filtered and the filter cake was washed with MeOH (5 mL*3), the filtrate was concentrated to give the crude. The residue was dissolved in DCM (10 mL) and dried over Na₂SO₄, filtered and concentrated to give a product. The crude product was used in the next step without further purification. Intermediate 1-4 (100 mg, 169.29 μmol, 62.37% yield) was obtained as a white solid. LCMS (Method D): Retention time: 0.274 min, [M+H]⁺=591.3.

### Step 3: Synthesis of I-254

To a solution of Intermediate 1-4 (70 mg, 118.51 μmol, 1 eq) and Intermediate 1-5 (48.77 mg, 118.51 μmol, 1 eq) in MeOH (1 mL) was added HOAc (7.12 mg, 118.51 μmol, 6.78 μL, 1 eq) and the mixture was stirred for 0.5 hr. Then NaBH₃CN (14.89 mg, 237.01 μmol, 2 eq) was added into the system. The mixture was stirred at 25° C. for 1 hr. The reaction mixture was diluted with H₂O (2 mL) and extracted with EA (5 mL*3). The combined organic layers were dried over Na₂SO₄, filtered and concentrated under reduced pressure to give a residue. The residue was purified by prep-HPLC (neutral condition) and the eluent was concentrated and lyophilized to give the desired product. I-254 (19.05 mg, 19.29 μmol, 16.28% yield, 99.855% purity) was obtained as a white solid. LCMS (Method H): Retention time: 0.601 min, [M+H]⁺=986.6. SFC: Retention time: 1.664, 1.979 min. ¹H NMR (400 MHz, METHANOL-d₄) δ=8.37 (d, J=7.6 Hz, 1H), 8.00-7.92 (m, 1H), 7.90-7.82 (m, 2H), 7.53-7.34 (m, 2H), 7.21-7.12 (m, 1H), 4.89 (s, 1H), 4.65-4.54 (m, 1H), 4.43-4.31 (m, 4H), 4.02-3.97 (m, 1H), 3.89-3.82 (m, 1H), 3.79 (d, J=3.2 Hz, 1H), 3.76-3.70 (m, 3H), 3.67 (s, 1H), 3.59-3.49 (m, 4H), 3.26-3.17 (m, 2H), 2.96-2.92 (m, 2H), 2.90-2.73 (m, 4H), 2.55 (s, 2H), 2.44 (d, J=8.8 Hz, 1H), 2.39 (s, 3H), 2.34-2.21 (m, 5H), 2.16 (s, 6H), 2.05-1.96 (m, 3H), 1.94-1.81 (m, 7H), 1.80-1.67 (m, 4H), 1.65-1.49 (m, 4H). ¹⁹F NMR (376 MHz, METHANOL-d₄) δ=−120.76.

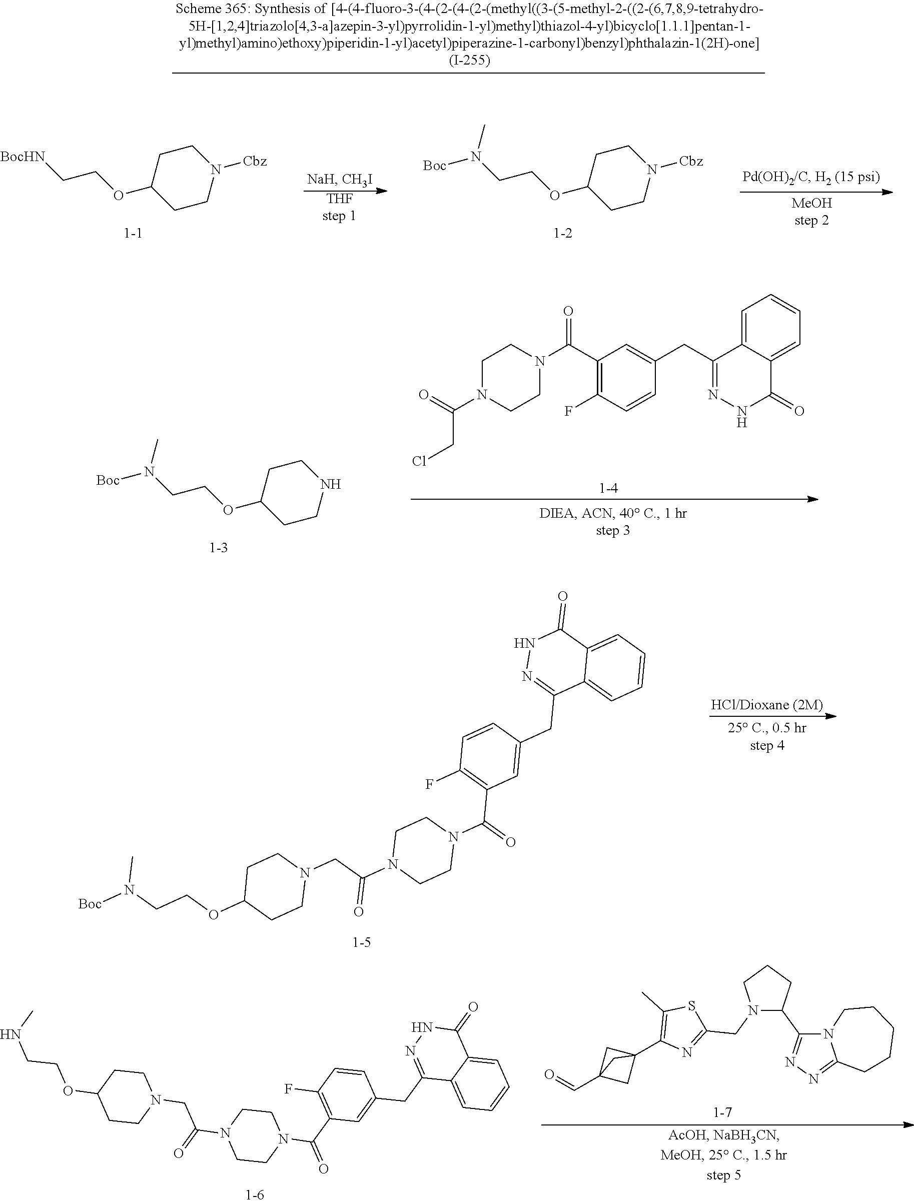

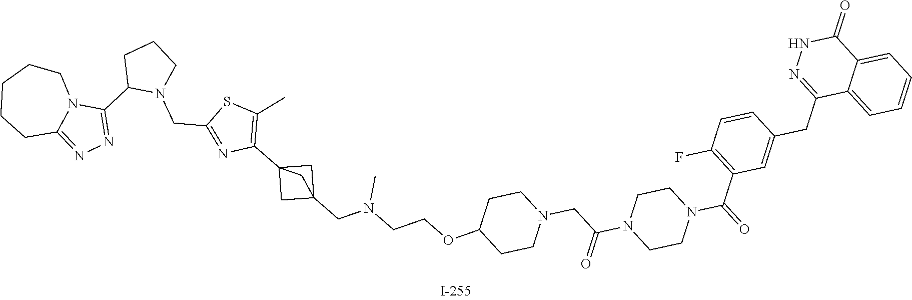 [SMILES: Cc1sc(CN2CCCC2c2nnc3n2CCCCC3)nc1C12CC(CN(C)CCOC3CCN(CC(=O)N4CCN(C(=O)c5cc(Cc6n[nH]c(=O)c7ccccc67)ccc5F)CC4)CC3)(C1)C2]

### Step 1: Synthesis of Intermediate 1-2.

To a solution of Intermediate 1-1 (600 mg, 1.59 mmol, 1 eq) in THF (6 mL) was added NaH (95.11 mg, 2.38 mmol, 60% purity, 1.5 eq) and the mixture was stirred at 0° C. for 0.1 hr. Then Mel (337.54 mg, 2.38 mmol, 148.04 μL, 1.5 eq) was added into the system. The mixture was stirred at 40° C. for 2 hr. The reaction mixture was quenched by H₂O (10 mL), and then extracted with EA (15 mL*3). The combined organic layers were dried over Na₂SO₄, filtered and concentrated under reduced pressure to give a residue. The crude product was used in the next step without further purification. Intermediate 1-2 (600 mg, 1.31 mmol, 82.54% yield, 85.598% purity) was obtained as a yellow solid. LCMS (Method D): Retention time: 0.474 min, [M+Na]⁺=415.2. ¹H NMR (400 MHz, METHANOL-d₄) δ=7.37-7.14 (m, 5H), 5.03 (s, 2H), 3.68-3.58 (m, 2H).

### Step 2: Synthesis of Intermediate 1-3.

To a solution of Pd(OH)₂/C (500 mg, 254.78 μmol, 20% purity) in MeOH (6 mL) was added Intermediate 1-2 (600 mg, 1.53 mmol, 1 eq) and the mixture was degassed and purged with H₂ 3 times, and then the mixture was stirred at 25° C. for 1 hr under H₂ (15 psi) atmosphere. The reaction mixture was filtered and washed with MeOH (10 mL*2) and the filtrate was concentrated under reduced pressure to give a product. The crude product was used in the next step without further purification. Intermediate 1-3 (350 mg, crude) was obtained as a colorless oil. LCMS (Method D): Retention time: 0.243 min, [M+H]⁺=259.2.

### Step 3: Synthesis of Intermediate 1-5.

To a solution of Intermediate 1-3 (300 mg, 1.16 mmol, 1 eq) and Intermediate 1-4 (514.26 mg, 1.16 mmol, 1 eq) in ACN (3 mL) was added DIEA (450.22 mg, 3.48 mmol, 606.77 μL, 3 eq). The mixture was stirred at 40° C. for 1 hr. The reaction mixture was concentrated under reduced pressure to give a residue. The residue was purified by flash silica gel chromatography (ISCO; 20 g SepaFlash Silica Flash Column, Eluent of 0˜11% Methanol/Ethyl acetate gradient @60 mL/min), the eluent was concentrated under reduced pressure to give a residue. Intermediate 1-5 (500 mg, 655.65 μmol, 56.46% yield, 87.170% purity) was obtained as a white solid. LCMS (Method D): Retention time: 0.338 min, [M+H]⁺=665.2. ¹H NMR (400 MHz, METHANOL-d₄) δ=8.39 (d, J=7.2 Hz, 1H), 8.00-7.95 (m, 1H), 7.93-7.82 (m, 2H), 7.55-7.36 (m, 2H), 7.22-7.12 (m, 1H), 4.41 (s, 2H), 3.84-3.68 (m, 4H), 3.61-3.52 (m, 4H), 3.40 (s, 5H), 3.30-3.22 (m, 2H), 2.93 (s, 3H), 2.82-2.69 (m, 2H), 2.42-2.25 (m, 2H), 1.97-1.85 (m, 2H), 1.70-1.54 (m, 2H), 1.47 (d, J=3.2 Hz, 9H).

### Step 4: Synthesis of Intermediate 1-6.

To a solution of Intermediate 1-5 (150 mg, 225.64 μmol, 1 eq) in DCM (0.5 mL) was added HCl/dioxane (2 M, 0.5 mL). The mixture was stirred at 25° C. for 0.5 hr. The reaction mixture was concentrated under reduced pressure to give a residue. The residue was purified by prep-HPLC (basic condition), and the eluent was concentrated and lyophilized to give the desired product. Intermediate 1-6 (120 mg, 183.82 μmol, 81.46% yield, 86.493% purity) was obtained as a white solid. LCMS (Method D): Retention time: 0.218 min, [M+H]⁺=565.3.

### Step 5: Synthesis of I-255

To a solution of Intermediate 1-6 (100 mg, 177.10 μmol, 1 eq) and Intermediate 1-7 (72.89 mg, 177.10 μmol, 1 eq) in MeOH (1 mL) was added HOAc (10.64 mg, 177.10 μmol, 10.14 μL, 1 eq) and the mixture was stirred for 0.5 hr. Then NaBH₃CN (22.26 mg, 354.20 μmol, 2 eq) was added into the system. The mixture was stirred at 25° C. for 1 hr. The reaction mixture was diluted with H₂O (2 mL) and extracted with EA (5 mL*3). The combined organic layers were concentrated under reduced pressure to give a residue. The residue was purified by prep-HPLC (neutral condition), the eluent was concentrated and lyophilized to give the desired product. I-255 (10.77 mg, 11.15 μmol, 6.29% yield, 99.390% purity) was obtained as a white solid. LCMS (Method H): Retention time: 0.596 min, [M+H]⁺=960.7. SFC: Retention time: 2.073, 2.460 min. ¹H NMR (400 MHz, METHANOL-d₄) δ=8.40-8.33 (m, 1H), 7.99-7.92 (m, 1H), 7.89-7.77 (m, 2H), 7.53-7.45 (m, 1H), 7.40-7.35 (m, 1H), 7.20-7.11 (m, 1H), 4.43-4.29 (m, 4H), 4.02-3.96 (m, 1H), 3.89-3.82 (m, 1H), 3.81-3.70 (m, 4H), 3.68-3.58 (m, 4H), 3.58-3.49 (m, 2H), 3.28-3.18 (m, 3H), 2.97-2.89 (m, 2H), 2.80-2.63 (m, 6H), 2.46-2.34 (m, 8H), 2.32-2.22 (m, 3H), 2.17 (d, J=4.0 Hz, 6H), 2.04-1.86 (m, 8H), 1.77 (d, J=1.2 Hz, 2H), 1.72-1.57 (m, 4H). ¹⁹F NMR (376 MHz, METHANOL-d₄) δ=−120.78.

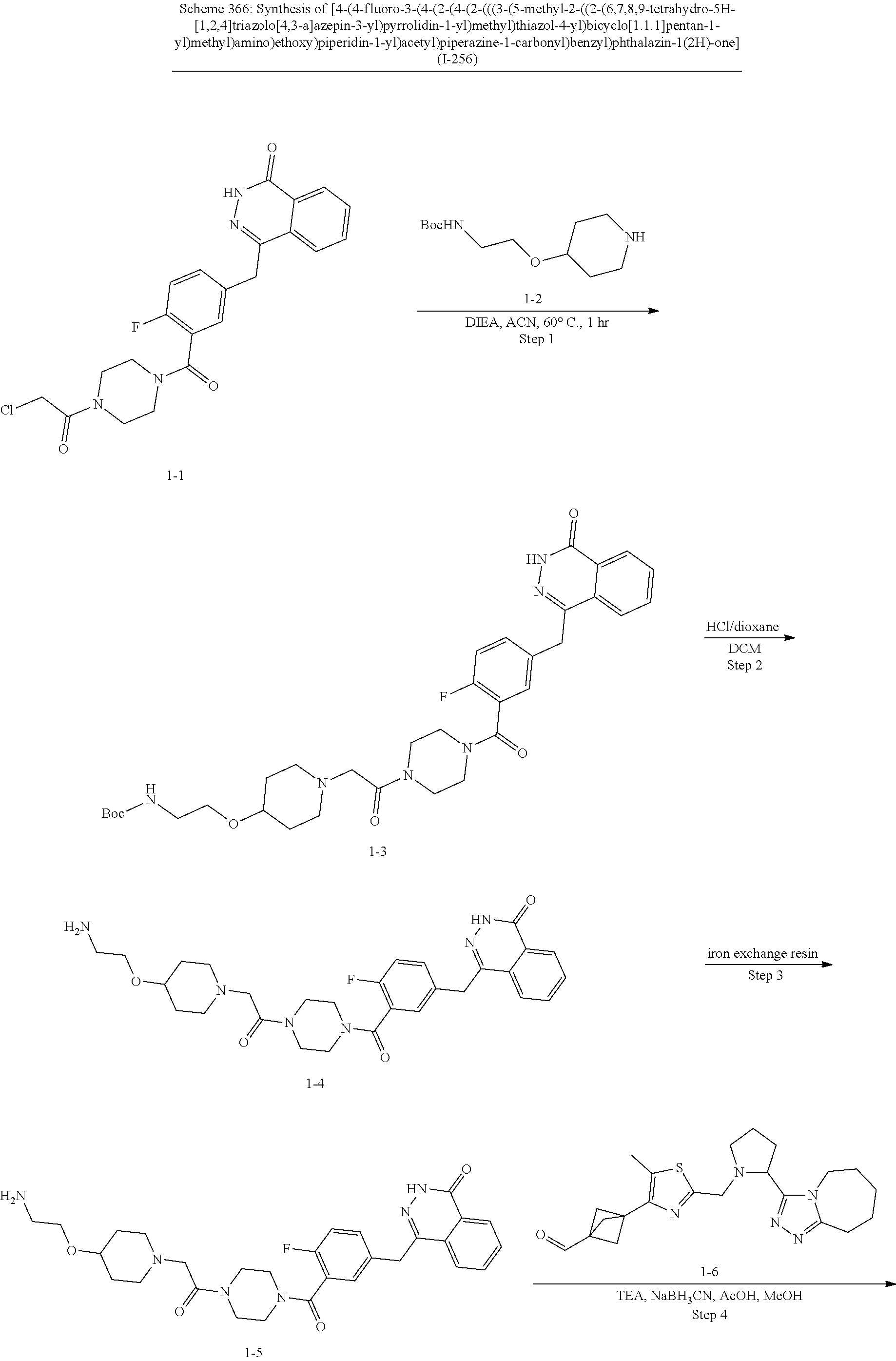 [SMILES: O=C(CCl)N1CCN(C(=O)c2cc(Cc3n[nH]c(=O)c4ccccc34)ccc2F)CC1 ; CC(C)(C)OC(=O)NCCOC1CCNCC1 ; CC(C)(C)OC(=O)NCCOC1CCN(CC(=O)N2CCN(C(=O)c3cc(Cc4n[nH]c(=O)c5ccccc45)ccc3F)CC2)CC1 ; NCCOC1CCN(CC(=O)N2CCN(C(=O)c3cc(Cc4n[nH]c(=O)c5ccccc45)ccc3F)CC2)CC1 ; NCCOC1CCN(CC(=O)N2CCN(C(=O)c3cc(Cc4n[nH]c(=O)c5ccccc45)ccc3F)CC2)CC1 ; Cc1sc(CN2CCCC2c2nnc3n2CCCCC3)nc1C12CC(C=O)(C1)C2]

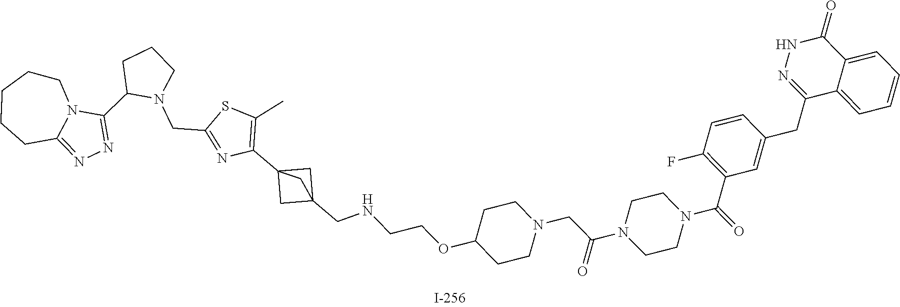 [SMILES: Cc1sc(CN2CCCC2c2nnc3n2CCCCC3)nc1C12CC(CNCCOC3CCN(CC(=O)N4CCN(C(=O)c5cc(Cc6n[nH]c(=O)c7ccccc67)ccc5F)CC4)CC3)(C1)C2]

### Step 1: Synthesis of Intermediate 1-3.

To a solution of Intermediate 1-2 (80 mg, 327.43 μmol, 1 eq) in ACN (1.5 mL) was added Intermediate 1-1 (145.01 mg, 327.43 μmol, 1 eq) and DIEA (126.95 mg, 982.28 μmol, 171.10 μL, 3 eq). The mixture was stirred at 60° C. for 1 hr. The reaction mixture was diluted with H₂O (2 mL) and extracted with EA (2 mL*3). The combined organic layers were dried over Na₂SO₄, filtered and concentrated under reduced pressure to give a residue. The residue was purified by flash silica gel chromatography (ISCO; 20 g SepaFlash Silica Flash Column, Eluent of 15% MeOH/EA gradient @60 mL/min). The eluent was concentrated under reduced pressure to give a residue. Intermediate 1-3 (110 mg, 149.76 μmol, 45.74% yield, 88.597% purity) was obtained as brown oil. LCMS (Method D): Retention time: 0.345 min, [M+H]⁺=651.2. ¹H NMR (400 MHz, CHLOROFORM-d) δ=10.36-10.05 (m, 1H), 8.51-8.42 (m, 1H), 7.83-7.69 (m, 3H), 7.33 (d, J=5.6 Hz, 2H), 7.07 (m, 1H), 4.29 (s, 2H), 3.95-3.65 (m, 4H), 3.63-3.45 (m, 4H), 3.40-3.11 (m, 7H), 2.76 (d, J=3.2 Hz, 2H), 2.32-2.17 (m, 2H), 2.10 (s, 1H), 1.94-1.87 (m, 2H), 1.62 (m, 3.6 Hz, 2H), 1.50-1.40 (m, 9H). ¹⁹F NMR (376 MHz, CHLOROFORM-d) δ=−117.537, −117.582.

### Step 2: Synthesis of Intermediate 1-4.

To a solution of Intermediate 1-3 (100 mg, 153.67 μmol, 1 eq) in DCM (0.5 mL) was added HCl/dioxane (2 M, 1 mL). The mixture was stirred at 25° C. for 0.5 hr. The reaction mixture was concentrated under reduced pressure to give a residue. The crude product was used for the next step directly. Intermediate 1-4 (190 mg, crude, HCl salt) was obtained as a white solid. LCMS (Method D): Retention time: 0.231 min, [M+H]⁺=551.2.

### Step 3: Synthesis of Intermediate 1-2.

To a solution of Intermediate 1-4 (180 mg, 306.60 μmol, 1 eq, HCl salt) in MeOH (4 mL) was added ion exchange resin (1 g). The mixture was stirred at 25° C. for 0.1 hr. The reaction mixture was stirred until the pH reached 8-9. The mixture was filtered and the filter cake was washed with MeOH (5 mL*3), the filtrate was concentrated to give the crude. The residue was dissolved in DCM (10 mL) and dried over Na₂SO₄, filtered and concentrated to give a product. The crude product was used in the next step without further purification. Intermediate 1-5 (70 mg, 127.13 μmol, 41.46% yield) was obtained as a white solid.

### Step 4: Synthesis of I-256

To a solution of Intermediate 1-5 (35 mg, 63.56 μmol, 1 eq) and Intermediate 1-3 (26.16 mg, 63.56 μmol, 1 eq) in MeOH (0.5 mL) was added TEA (38.59 mg, 381.39 μmol, 53.08 μL, 6 eq) and HOAc (22.90 mg, 381.39 μmol, 21.83 μL, 6 eq) and the mixture was stirred for 6 hr. Then NaBH₃CN (15.98 mg, 254.26 μmol, 4 eq) was added into the system. The mixture was stirred at 25° C. for 1 hr. The reaction mixture was diluted with H₂O (5 mL) and extracted with EA (5 mL*3). The combined organic layers were dried over Na₂SO₄, filtered and concentrated under reduced pressure to give a residue. The residue was purified by prep-HPLC (column: CD01-Phenomenex luna C18 150*25*10 um; mobile phase: [water (FA)-ACN]; gradient: 6%-36% B over 10 min), the eluent was concentrated and lyophilized to give a product. I-256 (8.25 mg, 8.16 μmol, 12.83% yield, 98.118% purity, FA salt) was obtained as a white solid. LCMS (Method D): Retention time: 0.291 min, [M+H]⁺=946.5. SFC: Retention time: 3.826, 5.221 min. ¹H NMR (400 MHz, METHANOL-d₄) δ=8.46 (d, J=4.0 Hz, 1H), 8.37 (d, J=6.4 Hz, 1H), 8.01-7.78 (m, 3H), 7.51 (s, 1H), 7.37 (s, 1H), 7.22-7.10 (m, 1H), 4.39 (s, 4H), 4.00 (s, 1H), 3.90-3.48 (m, 15H), 3.27-3.19 (m, 3H), 3.04 (s, 2H), 2.94 (s, 2H), 2.62 (d, J=2.4 Hz, 2H), 2.40 (s, 5H), 2.26 (s, 7H), 2.09-1.87 (m, 8H), 1.83-1.63 (m, 6H). ¹⁹F NMR (376 MHz, METHANOL-d₄) δ=−120.77.

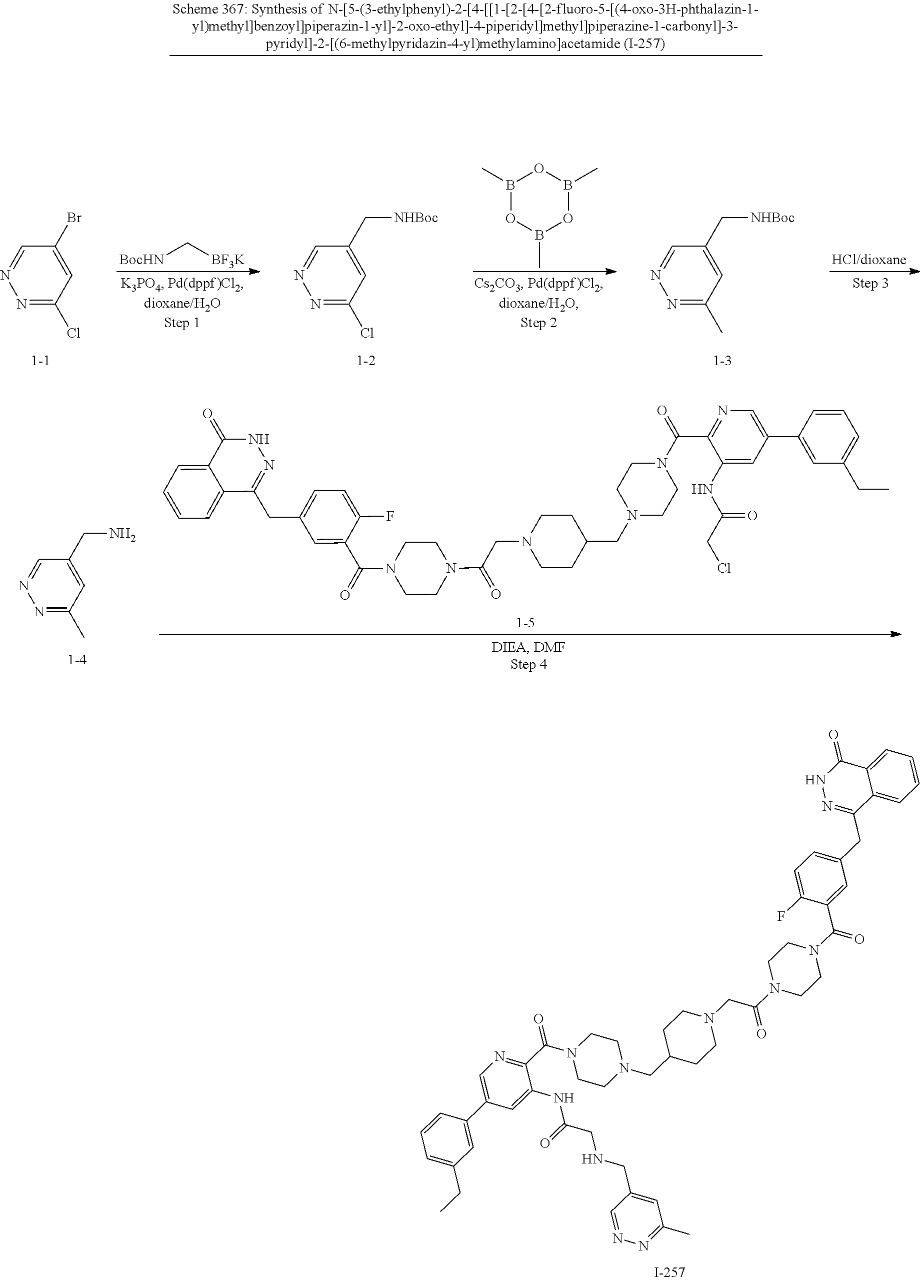

### Step 1: Synthesis of Intermediate 1-2.

A mixture of Intermediate 1-1 (6.7 g, 34.64 mmol, 1 eq) and potassium;(tert-butoxycarbonylamino)methyl-trifluoro-boranuide (9.85 g, 41.57 mmol, 1.2 eq) was added Pd(dppf)Cl₂ (2.53 g, 3.46 mmol, 0.1 eq), K₃PO₄ (22.06 g, 103.91 mmol, 3 eq) in dioxane (80 mL) and H₂O (16 mL) was degassed and purged with N₂ 3 times, and then the mixture was stirred at 90° C. for 16 hr under N₂ atmosphere. The reaction mixture was diluted with water (100 mL) and extracted with EA (20 mL*3). The combined organic layers were concentrated under reduced pressure to give a residue. The residue was purified by flash silica gel chromatography ((ISCO; 80 g SepaFlash Silica Flash Column, Eluent of 0-50% Ethyl acetate/Petroleum ether gradient @100 mL/min). The eluent was concentrated under reduced pressure to give Intermediate 1-2 (1.8 g, crude) as a white solid. LCMS (Method D): Retention time: 0.303 min, [M+H]⁺=244.0. ¹H NMR (400 MHz, CHLOROFORM-d) δ=9.05 (d, J=2.0 Hz, 1H), 7.47-7.43 (m, 1H), 5.16 (s, 1H), 4.37 (d, J=6.0 Hz, 2H), 1.47 (s, 9H).

### Step 2: Synthesis of Intermediate 1-3.

To a mixture of Intermediate 1-2 (0.5 g, 2.05 mmol, 1 eq) in dioxane (10 mL) and H₂O (2 mL) was added Pd(dppf)Cl₂ (150.13 mg, 205.18 μmol, 0.1 eq), Cs₂CO₃ (2.01 g, 6.16 mmol, 3 eq), and 2,4,6-trimethyl-1,3,5,2,4,6-trioxatriborinane (3.5 M, 2.93 mL, 5 eq), then the mixture was stirred at 80° C. for 16 hours. The reaction mixture was diluted with water (50 mL) and extracted with EA (20 mL*2). The combined organic layers were concentrated under reduced pressure to give a residue. The residue was purified by flash silica gel chromatography (ISCO; 40 g SepaFlash Silica Flash Column, Eluent of 0˜50% Ethyl acetate/Petroleum ether gradient @80 mL/min). The eluent was concentrated under reduced pressure to give Intermediate 1-3 (150 mg, 544.18 μmol, 26.52% yield, 81% purity) as a yellow solid. LCMS (Method D): Retention time: 0.294 min, [M+H]+=224.2.

### Step 3: Synthesis of Intermediate 1-4.

To a mixture of Intermediate 1-3 (80 mg, 358.31 μmol, 1 eq) in DCM (0.1 mL) was added HCl/dioxane (2 M, 895.77 μL, 5 eq), and then the mixture was stirred at stirred at 20° C. for 5 min. The reaction mixture was concentrated under reduced pressure to give Intermediate 1-4 (60 mg, crude, HCl salt) as a white solid. LCMS (Method D): Retention time: 0.053 min, [M+H]+=124.0.

### Step 4: Synthesis of I-257

To a mixture of Intermediate 1-5 (46.49 mg, 52.21 μmol, 1 eq) and Intermediate 1-4 (50 mg, 313.25 μmol, 6 eq, HCl salt) in DMF (1 mL) was added DIEA (20.24 mg, 156.63 μmol, 27.28 μL, 3 eq), and then the mixture was stirred at 40° C. for 1 hour. The reaction mixture was quenched with water (30 mL) and extracted with EA (5 mL*3). The combined organic layers were concentrated under reduced pressure to give a residue. The crude product was purified by reversed-phase HPLC (0.1% FA condition). The eluent was concentrated under reduced pressure to remove MeCN and then lyophilized to give I-257 (15.63 mg, 15.28 μmol, 29.26% yield, 100% purity, FA salt) as a yellow solid. LCMS (Method D): Retention time: 0.301 min, [M+H]+=977.3. ¹H NMR (400 MHz, METHANOL-d₄) δ=9.21 (d, J=1.6 Hz, 1H), 8.86 (d, J=2.0 Hz, 1H), 8.60 (d, J=2.0 Hz, 1H), 8.39 (s, 1H), 8.38-8.36 (m, 1H), 7.99-7.92 (m, 1H), 7.90-7.78 (m, 3H), 7.54-7.49 (m, 3H), 7.43 (t, J=7.6 Hz, 1H), 7.40-7.34 (m, 1H), 7.32 (d, J=7.6 Hz, 1H), 7.17 (t, J=9.2 Hz, 1H), 4.39 (s, 2H), 3.98 (s, 1H), 3.93 (s, 2H), 3.88 (s, 1H), 3.84-3.75 (m, 4H), 3.71 (d, J=3.6 Hz, 1H), 3.60-3.53 (m, 4H), 3.51 (s, 2H), 3.44-3.32 (m, 5H), 2.78-2.74 (m, 4H), 2.70 (s, 3H), 2.48 (s, 4H), 2.30-2.28 (m, 2H), 1.97 (d, J=6.0 Hz, 2H), 1.88-1.75 (m, 1H), 1.53-1.40 (m, 2H), 1.29 (t, J=7.6 Hz, 3H). ¹⁹F NMR (376 MHz, METHANOL-d₄) δ=−120.77.

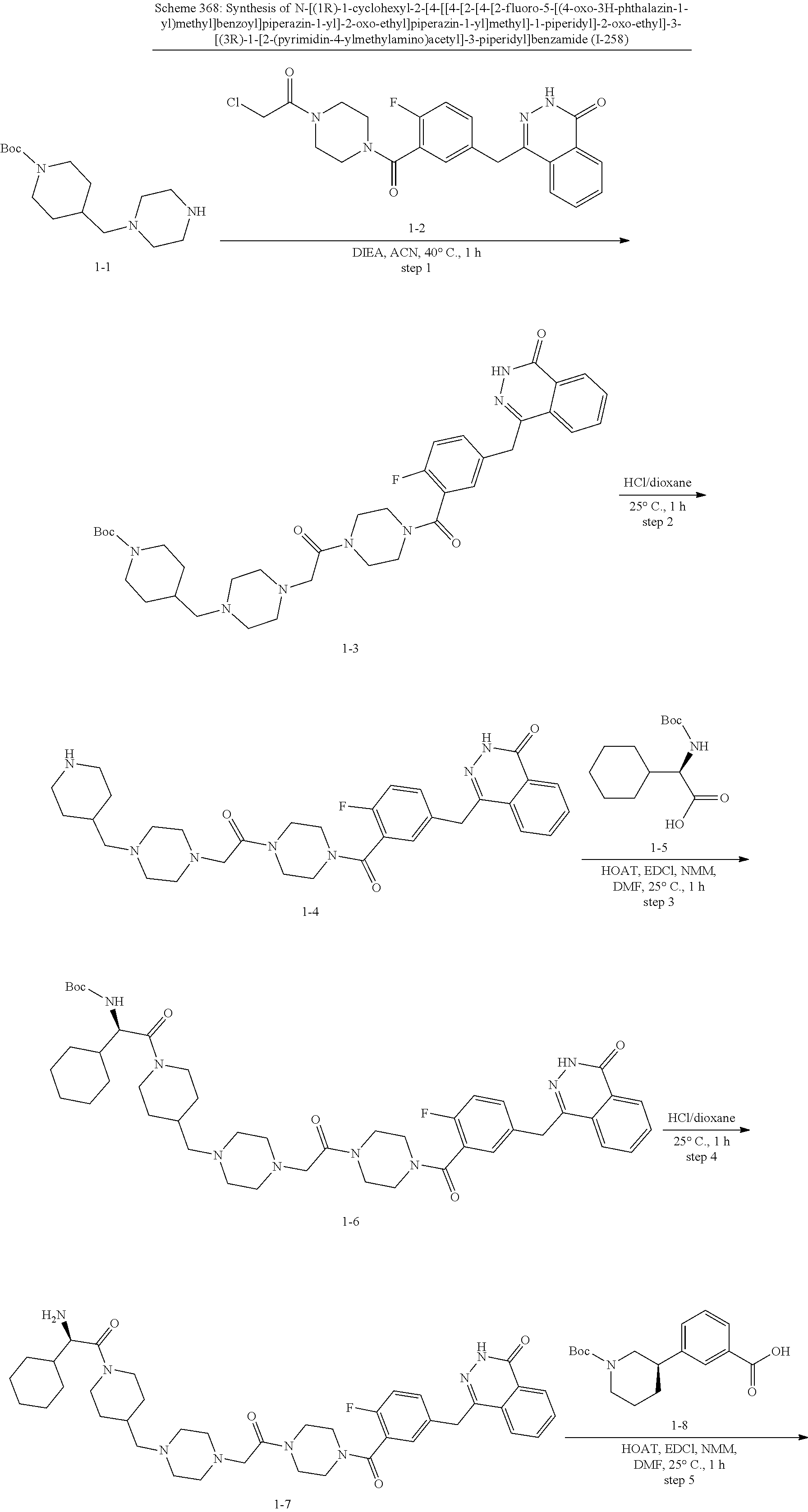

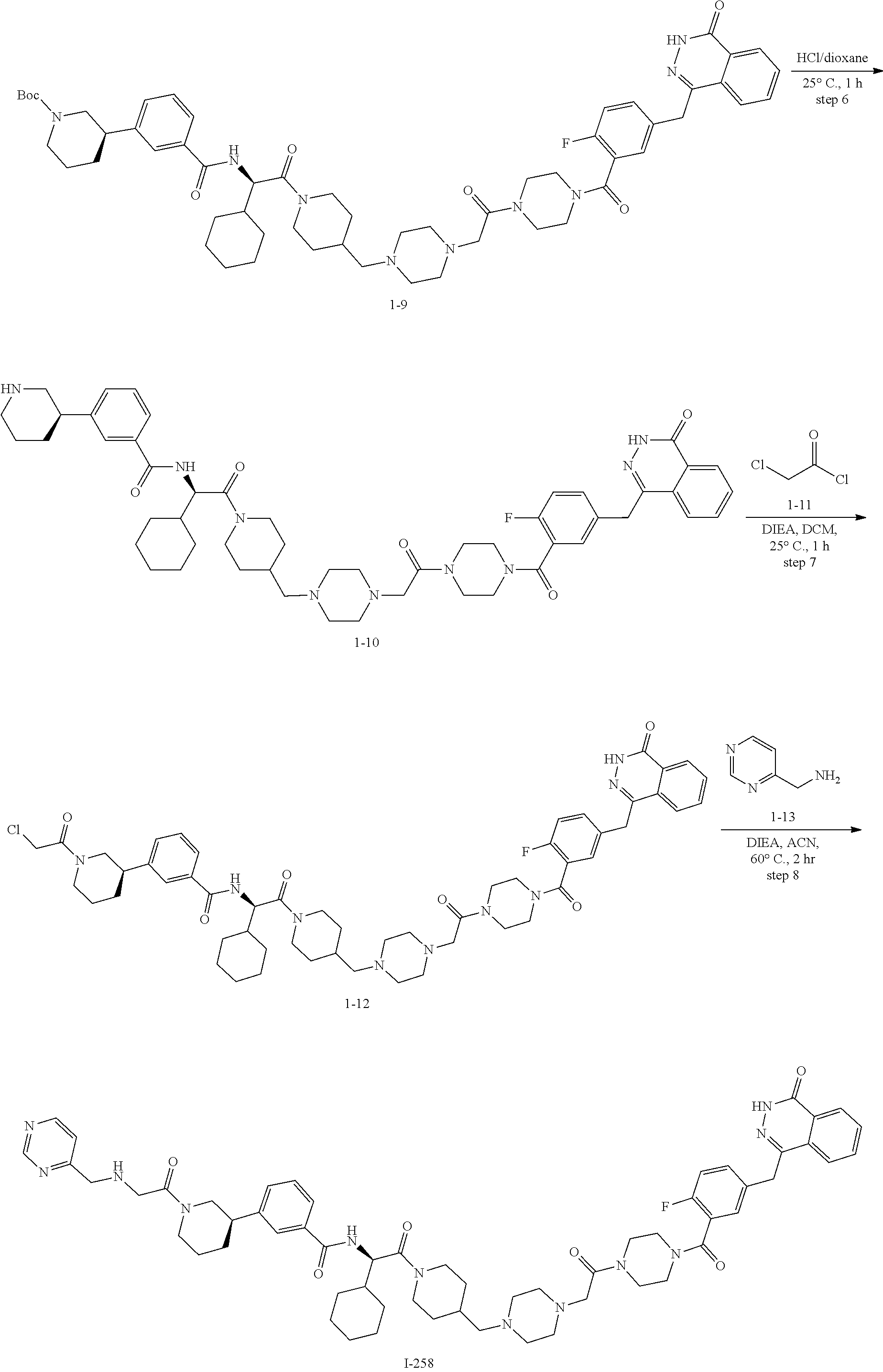

### Step 1: Synthesis of Intermediate 1-3

To a mixture of Intermediate 1-1 (3.20 g, 11.29 mmol, 1 eq) and Intermediate 1-2 (5 g, 11.29 mmol, 1 eq) in ACN (50 mL) was added DIEA (5.84 g, 45.16 mmol, 7.87 mL, 4 eq). The mixture was stirred at 40° C. for 1 hr. The reaction was concentrated under reduced pressure and poured into H₂O (15 mL) and extracted with EA (20 mL*3). The combined organic layers were dried over Na₂SO₄, filtered and concentrated under reduced pressure to give the crude product. The residue was purified by flash silica gel chromatography (ISCO; 80 g SepaFlash Silica Flash Column, Eluent of 0-40% Ethyl acetate/Methanol gradient @100 mL/min) and concentrated under reduced pressure to give a residue. Intermediate 1-3 (6.1 g, 8.84 mmol, 78.33% yield) was obtained as a yellow solid. LCMS (Method D): Rt=0.358 min, [M+H]+=690.3. ¹H NMR (400 MHz, METHANOL-d₄) δ=8.41-8.34 (m, 1H), 7.95 (d, J=4.8 Hz, 1H), 7.91-7.81 (m, 2H), 7.49 (d, J=1.2 Hz, 1H), 7.38 (d, J=5.2 Hz, 1H), 7.16 (t, J=8.8 Hz, 1H), 4.69-4.54 (m, 1H), 4.39 (s, 2H), 4.07-3.99 (m, 2H), 3.82-3.64 (m, 4H), 3.54 (d, J=18.4 Hz, 2H), 3.39-3.34 (m, 1H), 3.27 (s, 1H), 3.21 (s, 1H), 2.83-2.69 (m, 3H), 2.52 (s, 7H), 2.24-2.17 (m, 2H), 1.74 (d, J=11.1 Hz, 3H), 1.45 (s, 9H), 1.09-0.99 (m, 2H). ¹⁹F NMR (376 MHz, METHAN OL-d₄) δ=−120.787.

### Step 2: Synthesis of Intermediate 1-4

A mixture of Intermediate 1-3 (5.9 g, 8.55 mmol, 1 eq) in HCl/dioxane (2 M, 59.00 mL, 13.80 eq) was stirred at 25° C. for 1 hr. The reaction mixture was concentrated under reduced pressure to give a residue. Intermediate 1-4 (7.26 g, crude, HCl salt) was obtained as a yellow solid. LCMS (Method D): Rt=0.237 min, (M+H)=590.3. ¹H NMR (400 MHz, METHANOL-d₄) δ=8.37 (d, J=68 Hz, 1H), 7.99-7.93 (m, 1H), 7.92-7.80 (m, 2H), 7.54-7.47 (m, 1H), 7.42-7.35 (m, 1H), 7.17 (t, J=9.2 Hz, 1H), 4.68-4.50 (m, 2H), 4.39 (s, 2H), 3.99-3.88 (m, 4H), 3.88-3.77 (m, 4H), 3.76-3.69 (m, 2H), 3.61-3.52 (m, 2H), 3.50-3.39 (m, 4H), 3.38-3.33 (m, 2H), 3.30-3.16 (m, 1H), 3.07 (t, J=12.4 Hz, 2H), 2.40-2.26 (m, 1H), 2.26-2.15 (m, 2H), 1.67-1.60 (m, 1H), 1.60-1.50 (m, 2H). ¹⁹F NMR (376 MHz, METHANOL-d₄) δ=−120.725.

### Step 3: Synthesis of Intermediate 1-6

To a mixture of Intermediate 1-4 (2.26 g, 3.61 mmol, 1 eq, HCl salt) and Intermediate 1-5 (928.76 mg, 3.61 mmol, 1 eq) in DMF (22 mL) was added HOAT (491.26 mg, 3.61 mmol, 504.89 μL, 1 eq), EDCI (2.08 g, 10.83 mmol, 3 eq) and NMM (1.83 g, 18.05 mmol, 1.98 mL, 5 eq). The mixture was stirred at 25° C. for 1 hr. The reaction mixture was poured into H₂O (20 mL) and extracted with DCM (30 mL*3). The combined organic layers were dried over Na₂SO₄, filtered and concentrated under reduced pressure to give the crude product. The residue was purified by flash silica gel chromatography (ISCO; 80 g SepaFlash Silica Flash Column, Eluent of 0˜50% Ethyl acetate/Methanol gradient @60 mL/min) and concentrated under reduced pressure to give a residue. The residue was further purified by prep-HPLC (column: Phenomenex luna C18 150*25 mm*10 um; mobile phase: [water (FA)-ACN]; gradient: 15%-45% B over 8 min) followed by lyophilization to give a residue. Intermediate 1-6 (3 g, crude) was obtained as a yellow solid. LCMS (Method D): Rt=0.407 min, [M+H]+=829.4. SFC: Retention time: 1.188 min. ¹H NMR (400 MHz, METHANOL-d₄) δ=8.37 (d, J=7.6 Hz, 1H), 8.00-7.92 (m, 1H), 7.91-7.79 (m, 2H), 7.49 (s, 1H), 7.42-7.33 (m, 1H), 7.17 (t, J=8.8 Hz, 1H), 4.59 (s, 2H), 4.54-4.46 (m, 1H), 4.39 (s, 3H), 4.12 (d, J=7.6 Hz, 1H), 3.81-3.65 (m, 4H), 3.53 (d, J=15.2 Hz, 2H), 3.39 (s, 1H), 3.24 (s, 1H), 3.19-3.07 (m, 1H), 2.75-2.48 (m, 9H), 2.30-2.22 (m, 2H), 1.92-1.80 (m, 3H), 1.75 (d, J=8.4 Hz, 3H), 1.68-1.56 (m, 3H), 1.43 (d, J=3.2 Hz, 9H), 1.32-1.17 (m, 4H), 1.10-0.99 (m, 3H). ¹⁹F NMR (376 MHz, METHANOL-d₄) δ=−120.777.

### Step 4: Synthesis of Intermediate 1-7

A solution of Intermediate 1-6 (2.98 g, 3.34 mmol, 1 eq) in HCl/dioxane (2 M, 30 mL, 17.95 eq) was stirred at 25° C. for 1 hr. The reaction mixture was concentrated under reduced pressure to give a residue. Intermediate 1-7 (4.23 g, crude, HCl salt) was obtained as a light yellow solid. LCMS (Method D): Rt=0.312 min, [M+H]+=729.4.

### Step 5: Synthesis of Intermediate 1-9

To a mixture of Intermediate 1-7 (600 mg, 783.95 μmol, 1.1 eq, HCl salt) and Intermediate 1-8 (217.63 mg, 712.68 μmol, 1 eq) in DMF (6 mL) was added EDCI (409.86 mg, 2.14 mmol, 3 eq), HOAT (97.00 mg, 712.68 μmol, 99.70 μL, 1 eq) and NMM (360.43 mg, 3.56 mmol, 391.77 μL, 5 eq). The mixture was stirred at 25° C. for 1 hr. The reaction mixture was poured into H₂O (5 mL) and extracted with DCM (10 mL*3). The combined organic layers were dried over Na₂SO₄, filtered and concentrated under reduced pressure to give the crude product. The residue was purified by flash silica gel chromatography (ISCO; 20 g SepaFlash Silica Flash Column, Eluent of 0˜60% Ethyl acetate/Methanol gradient @40 mL/min) and concentrated under reduced pressure to give a residue. The residue was further purified by prep-HPLC (column: Phenomenex luna C18 150*25 mm*10 um; mobile phase: [water (FA)-ACN]; gradient: 28%-58% B over 10 min) followed by lyophilization to give a residue. Intermediate 1-9 (310 mg, 305.04 μmol, 42.80% yield) was obtained as a yellow solid. LCMS (Method D): Rt=0.479 min, [M+H]+=1016.5. SFC: Retention time: 0.648 min. ¹H NMR (400 MHz, METHANOL-d₄) δ=8.37 (d, J=7.2 Hz, 1H), 7.99-7.92 (m, 1H), 7.91-7.81 (m, 2H), 7.76-7.67 (m, 2H), 7.53-7.34 (m, 4H), 7.16 (t, J=9.2 Hz, 1H), 4.59 (s, 4H), 4.55-4.48 (m, 1H), 4.39 (s, 2H), 4.35-4.26 (m, 1H), 4.12 (d, J=12.4 Hz, 2H), 3.82-3.64 (m, 4H), 3.53 (d, J=14.0 Hz, 2H), 3.39-3.34 (m, 1H), 3.26-3.13 (m, 2H), 2.97-2.79 (m, 2H), 2.79-2.66 (m, 3H), 2.56 (d, J=3.6 Hz, 7H), 2.33-2.21 (m, 2H), 2.03-1.97 (m, 1H), 1.93-1.83 (m, 4H), 1.82-1.74 (m, 4H), 1.73-1.64 (m, 2H), 1.63-1.55 (m, 1H), 1.47 (s, 9H), 1.34-1.19 (m, 4H), 1.17-1.04 (m, 3H). ¹⁹F NMR (376 MHz, METHAN OL-d₄) δ=−120.792.

### Step 6: Synthesis of Intermediate 1-10

A mixture of Intermediate 1-9 (300 mg, 271.59 μmol, 1 eq) in HCl/dioxane (2 M, 3 mL, 22.09 eq) was stirred at 25° C. for 1 hr. The reaction mixture was concentrated under reduced pressure to give a residue. Intermediate 1-10 (450 mg, crude, HCl salt) was obtained as a light yellow solid. LCMS (Method D): Rt=0.321 min, [M+H]+=916.8.

### Step 7: Synthesis of Intermediate 1-12

To a mixture of Intermediate 1-10 (400 mg, 419.91 μmol, 1 eq, HCl salt) and Intermediate 1-11 (47.43 mg, 419.91 μmol, 33.45 μL, 1 eq) in DCM (4 mL) was added DIEA (108.54 mg, 839.81 μmol, 146.28 μL, 2 eq). The mixture was stirred at 25° C. for 1 hr. The reaction mixture was concentrated under reduced pressure to give a residue. The crude product was purified by reversed-phase HPLC (0.1% FA condition) followed by lyophilization to give a residue. Intermediate 1-12 (115 mg, 110.06 μmol, 26.21% yield, 95% purity) was obtained as a white solid. LCMS (Method D): Rt=0.376 min, [M+H]+=992.4. SFC: Retention time: 2.792 min. ¹H NMR (400 MHz, METHANOL-d₄) δ=8.39-8.35 (m, 1H), 7.99-7.92 (m, 1H), 7.91-7.80 (m, 2H), 7.80-7.68 (m, 2H), 7.54-7.41 (m, 3H), 7.40-7.33 (m, 1H), 7.17 (t, J=8.8 Hz, 1H), 4.95-4.88 (m, 2H), 4.63-4.49 (m, 4H), 4.39 (s, 2H), 4.37-4.30 (m, 2H), 3.99 (d, J=13.2 Hz, 1H), 3.83-3.72 (m, 2H), 3.67 (s, 2H), 3.55-3.45 (m, 3H), 3.41 (t, J=10.0 Hz, 1H), 3.34 (s, 1H), 3.28-3.12 (m, 4H), 2.95-2.69 (m, 10H), 2.61-2.48 (m, 2H), 2.09-2.03 (m, 1H), 1.87 (d, J=11.6 Hz, 5H), 1.81-1.75 (m, 2H), 1.73-1.66 (m, 2H), 1.36-1.18 (m, 5H), 1.16-1.05 (m, 3H). ¹⁹F NMR (376 MHz, METHAN OL-d₄) δ=−120.800.

### Step 8: Synthesis of I-258

To a mixture of Intermediate 1-12 (70 mg, 66.99 μmol, 1 eq) and Intermediate 1-13 (21.93 mg, 200.98 μmol, 3 eq) in ACN (1 mL) was added DIEA (25.98 mg, 200.98 μmol, 35.01 μL, 3 eq). The mixture was stirred at 60° C. for 2 h. The reaction mixture was concentrated under reduced pressure to give a residue. The crude product was purified by reversed-phase HPLC (0.1% FA condition) followed by lyophilization to give a residue. I-258 (6 mg, 5.24 μmol, 7.82% yield, 96.986% purity, FA salt) was obtained as a yellow solid. LCMS (Method D): Rt=0.318 min, [M+H]+=1065.5. SFC: Retention time: 1.609 min. ¹H NMR (400 MHz, METHANOL-d₄) δ=9.11 (d, J=9.2 Hz, 1H), 8.77-8.69 (m, 1H), 8.47 (s, 1H), 8.37 (d, J=7.2 Hz, 1H), 7.95 (d, J=6.8 Hz, 1H), 7.90-7.81 (m, 2H), 7.77 (s, 1H), 7.71 (t, J=6.0 Hz, 1H), 7.61-7.55 (m, 1H), 7.49 (d, J=7.2 Hz, 2H), 7.46-7.41 (m, 1H), 7.37 (d, J=1.2 Hz, 1H), 7.17 (t, J=8.8 Hz, 1H), 4.94 (s, 2H), 4.67-4.45 (m, 3H), 4.39 (s, 2H), 4.34-4.27 (m, 1H), 4.04 (d, J=15.2 Hz, 2H), 3.89-3.65 (m, 7H), 3.52 (d, J=1.2 Hz, 2H), 3.45-3.39 (m, 1H), 3.36 (s, 2H), 3.24-3.13 (m, 2H), 2.95-2.71 (m, 4H), 2.70-2.48 (m, 7H), 2.41-2.29 (m, 2H), 2.08-2.03 (m, 1H), 1.87 (d, J=6.0 Hz, 7H), 1.81-1.75 (m, 2H), 1.69 (d, J=1.6 Hz, 2H), 1.35-1.21 (m, 4H), 1.17-1.05 (m, 3H). ¹⁹F NMR (376 MHz, METHAN OL-d₄)

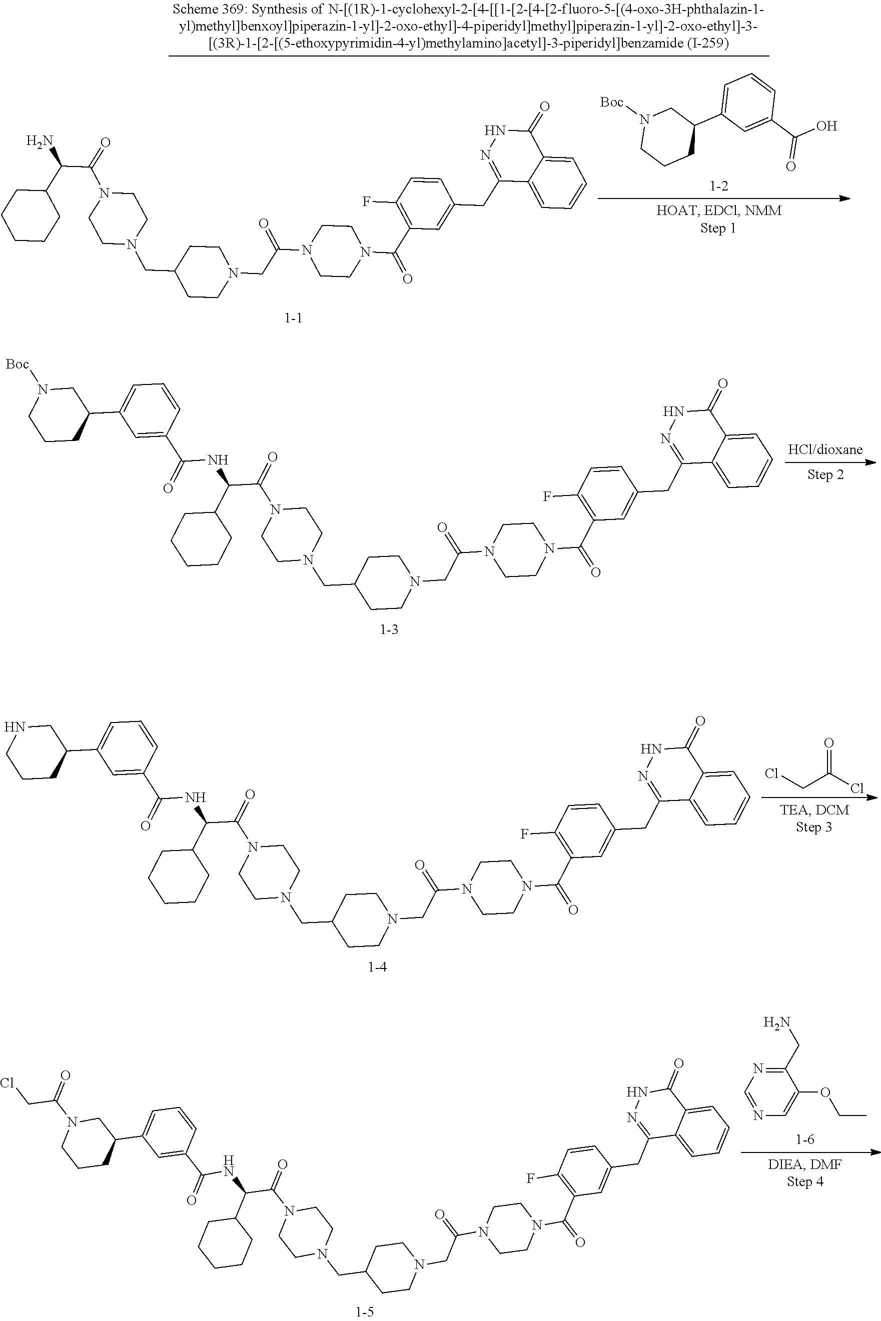

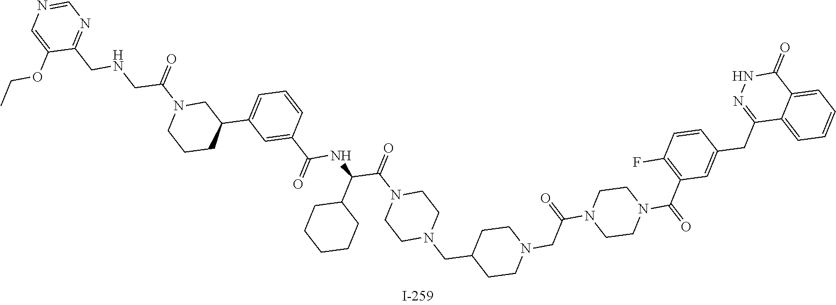 [SMILES: CCOc1cncnc1CNCC(=O)N1CCC[C@H](c2cccc(C(=O)N[C@@H](C(=O)N3CCN(CC4CCN(CC(=O)N5CCN(C(=O)c6cc(Cc7n[nH]c(=O)c8ccccc78)ccc6F)CC5)CC4)CC3)C3CCCCC3)c2)C1]

### Step 1: Synthesis of Intermediate 1-3

To a solution of Intermediate 1-2 (1.2 g, 3.93 mmol, 1 eq) in DMF (12 mL) was added EDCI (2.26 g, 11.79 mmol, 3 eq), NMM (1.99 g, 19.65 mmol, 2.16 mL, 5 eq) and HOAt (534.87 mg, 3.93 mmol, 549.72 L, 1 eq). Then Intermediate 1-1 (3.01 g, 3.93 mmol, 1 eq, HCl salt) was added into the mixture and it was stirred at 25° C. for 1.5 hr. The reaction mixture was diluted with water (60 mL) and extracted with ethyl acetate (20 mL×3). The combined organic layers were washed with brine (20 mL×3) and dried over sodium sulfate, filtered and concentrated under reduced pressure to give a residue. The crude product was purified by reversed-phase HPLC (0.1% FA condition). The eluent was concentrated under reduced pressure to remove ACN and then lyophilized to give Intermediate 1-3 (2.6 g, 2.52 mmol, 64.05% yield, 98.38% purity) as a yellow solid. LCMS (Method D): Rt: 0.434 min, [M+H]+=1016.4. SFC: Rt: 0.645 min. ¹H NMR (400 MHz, CHLOROFORM-d) δ=10.38-10.11 (m, 1H), 8.51-8.40 (m, 1H), 8.16 (s, 1H), 7.83-7.62 (m, 5H), 7.42-7.29 (m, 4H), 7.15-7.00 (m, 2H), 5.07 (s, 1H), 4.69-4.51 (m, 1H), 4.29 (s, 2H), 4.25-4.04 (m, 3H), 3.89-3.63 (m, 4H), 3.57 (s, 2H), 3.39-3.17 (m, 4H), 3.15-3.08 (m, 1H), 2.80-2.68 (m, 3H), 2.59 (d, J=2.8 Hz, 9H), 2.28-2.13 (m, 14H), 1.87-1.70 (m, 9H), 1.30-1.01 (m, 8H).

### Step 2: Synthesis of Intermediate 1-4

To a solution of Intermediate 1-3 (2.6 g, 2.56 mmol, 1 eq) in DCM (8 mL) was added HCl/dioxane (2 M, 4 mL, 3.13 eq). The mixture was stirred at 25° C. for 1 hr. The mixture was concentrated to give Intermediate 1-4 (2.5 g, crude, HCl salt) as a yellow solid. LCMS (Method D): Rt: 0.318 min, [M+H]+=916.5. SFC: Rt: 0.940 min.

### Step 3: Synthesis of Intermediate 1-5

To a solution of Intermediate 1-4 (2.5 g, 2.62 mmol, 1 eq, HCl salt) in DCM (25 mL) was added TEA (1.33 g, 13.12 mmol, 1.83 mL, 5 eq) at 0° C. Then 2-chloroacetyl chloride (355.69 mg, 3.15 mmol, 250.84 μL, 1.2 eq) was added dropwise into the mixture and it was stirred at 0° C. for 1 hr. The reaction mixture was diluted with water (30 mL) and extracted with DCM (10 mL×3). The combined organic layers were washed with brine (10 mL×3) and dried over sodium sulfate, filtered and concentrated under reduced pressure to give a residue. The crude product was purified by reversed-phase HPLC (H₂O). The eluent was concentrated under reduced pressure to remove ACN and then lyophilized to give Intermediate 1-5 (1.4 g, 1.37 mmol, 52.21% yield, 97.153% purity) as a yellow solid. LCMS (Method D): Rt: 0.393 min, [M+H]+=992.3. SFC: Rt: 3.138 min. ¹H NMR (400 MHz, CHLOROFORM-d) δ=9.95 (s, 1H), 8.47 (d, J=6.0 Hz, 1H), 7.83-7.65 (m, 5H), 7.42-7.29 (m, 4H), 7.13-7.02 (m, 2H), 5.09 (d, J=6.0 Hz, 1H), 4.75-4.54 (m, 2H), 4.29 (s, 2H), 4.20-4.04 (m, 3H), 3.98-3.88 (m, 1H), 3.85-3.66 (m, 4H), 3.65-3.54 (m, 2H), 3.42-3.00 (m, 7H), 2.94-2.58 (m, 4H), 2.57-2.34 (m, 8H), 2.24-2.07 (m, 3H), 1.76 (d, J=10.0 Hz, 9H), 1.29-1.05 (m, 8H).

### Step 4: Synthesis of I-259

To a solution of Intermediate 1-5 (200 mg, 201.49 μmol, 1 eq) in DMF (2 mL) was added Intermediate 1-6 (107.68 mg, 402.98 μmol, 2 eq, TFA salt) and DIEA (78.12 mg, 604.46 μmol, 105.29 L, 3 eq). The mixture was stirred at 40° C. for 1 hr. The reaction mixture was diluted with water (30 mL) and extracted with ethyl acetate (10 mL×3). The combined organic layers were washed with brine (10 mL×3) and dried over sodium sulfate, filtered and concentrated under reduced pressure to give a residue. The residue was purified by prep-HPLC (column: CD01-Phenomenex luna C18 150*25*10 um; mobile phase: [water (TFA)-ACN]; gradient: 14%-44% B over 10 min). The eluent was concentrated under reduced pressure to remove ACN and then lyophilized to give I-259 (51.34 mg, 40.58 μmol, 20.14% yield, 96.70% purity, TFA salt) as a yellow solid. LCMS (Method H): Rt: 0.616 min, [M+H]+=1109.7. SFC: Rt: 4.856 min. ¹H NMR (400 MHz, METHANOL-d₄) δ=8.80 (d, J=3.2 Hz, 1H), 8.53 (d, J=6.4 Hz, 1H), 8.36 (d, J=7.6 Hz, 1H), 7.98-7.92 (m, 1H), 7.91-7.70 (m, 4H), 7.53-7.42 (m, 3H), 7.40-7.34 (m, 1H), 7.17 (t, J=8.8 Hz, 1H), 4.98-4.91 (m, 2H), 4.60 (d, J=13.6 Hz, 1H), 4.54-4.49 (m, 2H), 4.46 (d, J=12.4 Hz, 1H), 4.38 (s, 2H), 4.36-4.25 (m, 4H), 3.93-3.79 (m, 3H), 3.78-3.67 (m, 3H), 3.60 (s, 1H), 3.53 (s, 1H), 3.44 (s, 1H), 3.35 (s, 5H), 3.28-3.17 (m, 6H), 3.15-3.13 (m, 1H), 3.00-2.98 (m, J=5.6 Hz, 1H), 2.97-2.64 (m, 4H), 2.21-2.03 (m, 2H), 1.89 (s, 6H), 1.77 (s, 2H), 1.72-1.61 (m, 3H), 1.49-1.46 (m, 3H), 1.37-1.21 (m, 4H), 1.20-1.08 (m, 3H). ¹⁹F NMR (377 MHz, METHANOL-d₄) δ=−77.142, −120.614.

### Scheme 370: Synthesis of N—((R)-1-cyclohexyl-2-(4-((1-(2-(4-(2-fluoro-5-((4-oxo-3,4-dihydrophthalazin-1-yl)methyl)benzoyl)piperazin-1-yl)-2-oxoethyl)piperidin-4-yl)methyl)piperazin-1-yl)-2-oxoethyl)-3-((R)-1-(((4-ethoxy-6-methoxypyridin-3-yl)methyl)glycyl)piperidin-3-yl)benzamide (I-260)

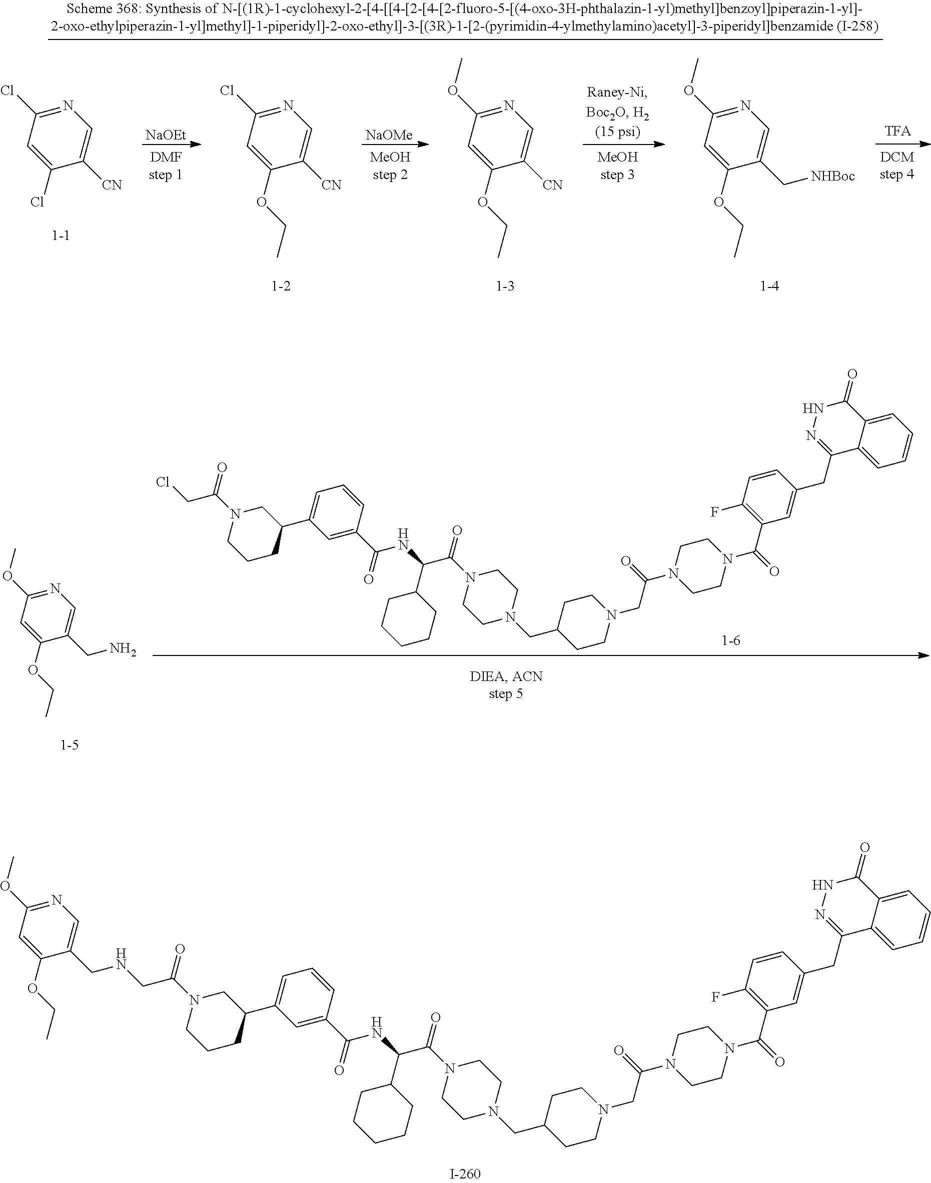

### Step 1: Synthesis of Intermediate 1-2

To a solution of Intermediate 1-1 (5 g, 28.90 mmol, 1 eq) in DMF (50 mL) was added NaOEt (9.83 g, 28.90 mmol, 1 eq). The mixture was stirred at 25° C. for 1 hr. The reaction mixture was diluted with water (40 mL) and extracted with EA (20 mL*3). The combined organic layers were dried over Na₂SO₄, filtered and concentrated under reduced pressure to give a residue. The residue was purified by flash silica gel chromatography (ISCO; 220 g SepaFlash Silica Flash Column, Eluent of 0˜100% Petroleum ether/Ethyl acetate @100 mL/min). The eluent was concentrated under reduced pressure to give Intermediate 1-2 (2.2 g, 12.05 mmol, 41.69% yield) as a yellow solid. LCMS (Method D): Rt=0.251 min, [M+H]+=183.1.

### Step 2: Synthesis of Intermediate 1-3

To a solution of Intermediate 1-2 (2.1 g, 11.50 mmol, 1 eq) in MeOH (21 mL) was added NaOMe (2.07 g, 11.50 mmol, 1 eq). The mixture was stirred at 25° C. for 3.5 hr. The reaction mixture was diluted with water (40 mL) and extracted with EA (20 mL*3). The combined organic layers were dried over Na₂SO₄, filtered and concentrated under reduced pressure to give a residue. The residue was purified by reversed phase HPLC (0.1% FA condition), the eluent was lyophilized to give Intermediate 1-3 (790 mg, 4.43 mmol, 38.55% yield) as a white solid. LCMS (Method D): Rt=0.342 min, [M+H]+=179.1.

### Step 3: Synthesis of Intermediate 1-4

To a solution of Intermediate 1-3 (300 mg, 1.68 mmol, 1 eq) and Boc₂O (404.20 mg, 1.85 mmol, 425.47 μL, 1.1 eq) in MeOH (5 mL) was added Raney-Ni (288.47 mg, 3.37 mmol, 2 eq) under N₂ atmosphere. The suspension was degassed and purged with H₂ 3 times. The mixture was stirred under H₂ (50 psi) at 25° C. for 5 hr. The reaction was diluted with MeOH (25 mL), and concentrated under reduced pressure to give a residue. The residue was purified by prep-HPLC (column: CD06-Waters Xbidge C18 150*40* 10 um; mobile phase: [water (NH₄HCO₃)-ACN]; gradient: 42%-72% B over 10 min). The eluent was lyophilized to give product. Intermediate 1-4 (95 mg, 336.48 μmol, 19.99% yield) was obtained as a yellow solid. LCMS (Method F): Rt=0.580 min, [M+H]+=283.0. ¹H NMR (400 MHz, METHANOL-d₄) δ=7.81 (s, 1H), 6.31 (s, 1H), 4.16-4.08 (m, 4H), 3.86 (s, 3H), 1.46-1.41 (m, 12H).

### Step 4: Synthesis of Intermediate 1-5

To a solution of Intermediate 1-4 (90 mg, 318.77 μmol, 1 eq) in DCM (0.6 mL) was added TFA (307.00 mg, 2.69 mmol, 200.00 μL, 8.45 eq). The mixture was stirred at 25° C. for 1.5 hr. The reaction mixture was concentrated under reduced pressure to give a residue. The residue was used in the next step without further purification. Intermediate 1-5 (110 mg, crude, TFA salt) was obtained as yellow oil. LCMS (Method F): Rt=0.398 min, [M+H]+=183.0. ¹H NMR (400 MHz, DMSO-d₆) δ=8.02 (s, 1H), 7.94 (s, 2H), 6.45 (s, 1H), 4.16-4.11 (m, 2H), 3.93-3.89 (m, 2H), 3.84 (s, 3H), 1.37 (t, J=7.2 Hz, 3H).

### Step 5: Synthesis of I-260

To a solution of Intermediate 1-5 (50 mg, 168.78 μmol, 1 eq, TFA salt) and Intermediate 1-6 (67.01 mg, 67.51 μmol, 0.4 eq) in ACN (0.5 mL) was added DIEA (43.63 mg, 337.56 μmol, 58.80 μL, 2 eq). The mixture was stirred at 25° C. for 1 hr. The reaction mixture was diluted with water (5 mL) and extracted with EA (5 mL*3). The combined organic layers were dried over Na₂SO₄, filtered and concentrated under reduced pressure to give a residue. The residue was purified by reversed phase column (0.1% NH₃·H₂O condition), and the eluent was lyophilized to give product. I-260 (8 mg, 7.03 μmol, 53.33% yield, TFA salt) was obtained as a white solid. LCMS (Method D): Rt=0.349 min, [M+H]+=1138.9. ¹H NMR (400 MHz, METHANOL-d₄) δ=8.37 (d, J=7.6 Hz, 1H), 7.99-7.93 (m, 1H), 7.92-7.80 (m, 3H), 7.79-7.67 (m, 2H), 7.53-7.42 (m, 3H), 7.41-7.34 (m, 1H), 7.17 (t, J=8.8 Hz, 1H), 6.37 (d, J=15.6 Hz, 1H), 4.59 (s, 4H), 4.39 (s, 2H), 4.33-4.27 (m, 1H), 4.18-4.13 (m, 2H), 3.87 (s, 4H), 3.81 (s, 3H), 3.76-3.70 (m, 2H), 3.69-3.60 (m, 3H), 3.53 (d, J=16.0 Hz, 3H), 3.25-3.15 (m, 3H), 2.60-2.45 (m, 7H), 2.23-2.17 (m, 3H), 2.08-1.98 (m, 2H), 1.91-1.76 (m, 9H), 1.72-1.65 (m, 2H), 1.51-1.40 (m, 4H), 1.36-1.24 (m, 10H), 1.15-1.05 (m, 3H). ¹⁹F NMR (376 MHz, METHANOL-d₄) δ=−76.962, −120.809. SFC: Retention time: 0.875 min.

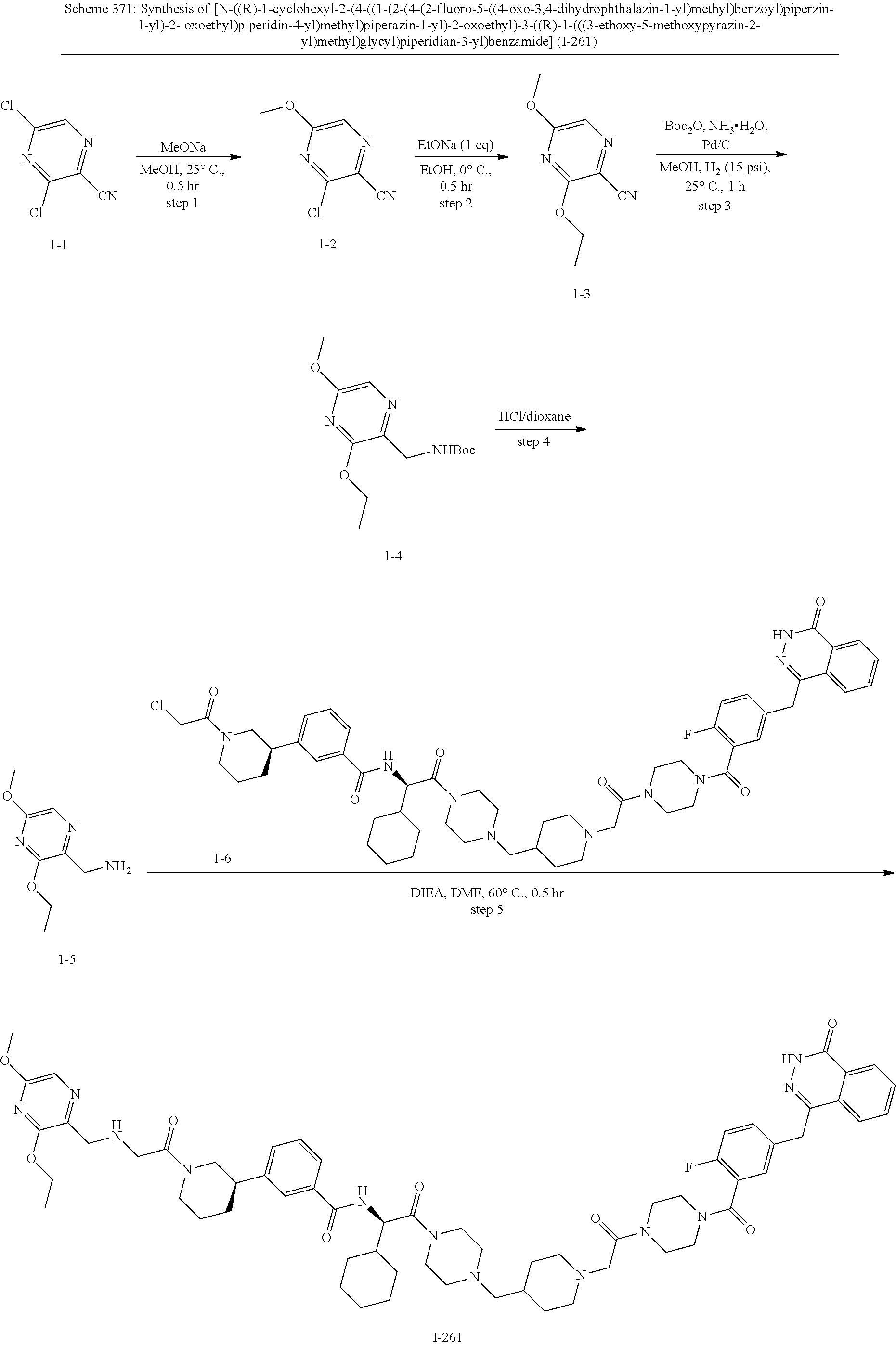

### Step 1: Synthesis of Intermediate 1-2

To a mixture of Intermediate 1-1 (2 g, 11.50 mmol, 1 eq) in MeOH (20 mL) was added sodium;methanolate (5 M, 2.30 mL, 1 eq) at 0° C., the mixture was stirred at 25° C. for 0.5 hr. The reaction mixture was filtered and concentrated under reduced pressure to give a residue. The residue was purified by flash silica gel chromatography (ISCO; 80 g SepaFlash Silica Flash Column, Eluent of 0˜50% Ethyl acetate/Petroleum ether gradient @100 mL/min) was concentrated to give Intermediate 1-2 (1.8 g, 10.62 mmol, 92.35% yield) as a white solid. LCMS (Method D): Retention time: 0.219 min, [M+H]+=170.1. ¹H NMR (400 MHz, CHLOROFORM-d) δ=8.20 (s, 1H), 4.09 (s, 3H).

### Step 2: Synthesis of Intermediate 1-3

To a solution of Intermediate 1-2 (1 g, 5.90 mmol, 1 eq) in EtOH (10 mL) was added EtONa (2.01 g, 5.90 mmol, 20% purity, 1 eq). The mixture was stirred at 0° C. for 0.5 hr. The reaction mixture was filtered and concentrated under reduced pressure to give a residue. The residue was purified by prep-HPLC (0.1% FA) and the eluent was concentrated to remove ACN and lyophilized to give Intermediate 1-3 (100 mg, 558.11 μmol, 9.46% yield) as a white solid. LCMS (Method D): Retention time: 0.288 min, [M+H]+=180.1. ¹H NMR (400 MHz, CHLOROFORM-d) δ=7.76 (s, 1H), 4.42-4.37 (m, 2H), 3.99 (s, 3H), 1.39-1.36 (m, 3H).

### Step 3: Synthesis of Intermediate 1-4

To a solution of Intermediate 1-3 (80 mg, 446.49 μmol, 1 eq), tert-butoxycarbonyl tert-butyl carbonate (146.17 mg, 669.73 μmol, 153.86 μL, 1.5 eq) in MeOH (1 mL) was added Pd/C (40.00 mg, 37.59 μmol, 10% purity, 8.42e−2 eq), NH₃·H₂O (125.18 mg, 892.98 μmol, 137.56 μL, 25% purity, 2 eq) under N₂ atmosphere. The suspension was degassed under vacuum and purged with H₂ 3 times. The mixture was stirred under H₂ (15 psi) at 25° C. for 1 hr. The reaction mixture was filtered and concentrated under reduced pressure to give a residue. The residue was purified by flash silica gel chromatography (ISCO; 12 g SepaFlash Silica Flash Column, Eluent of 0˜20% Ethyl acetate/Petroleum ether gradient @40 mL/min) and was concentrated under reduced pressure to give Intermediate 1-4 (50 mg, 168.87 μmol, 37.82% yield, 95.692% purity) as a white solid. LCMS (Method D): Retention time: 0.466 min, [M+H-tBu]+=227.9.

### Step 4: Synthesis of Intermediate 1-5

To a solution of Intermediate 1-4 (40 mg, 141.18 μmol, 1 eq) in DCM (0.2 mL) was added HCl/dioxane (2 M, 800.00 μL, 11.33 eq). The mixture was stirred at 25° C. for 0.5 hr. The reaction mixture was concentrated under reduced pressure to give Intermediate 1-5 (45 mg, crude, HCl salt) as a white solid. LCMS (Method D): Retention time: 1.193 min, [M+H]+=184.2.

### Step 5: Synthesis of I-261

To a solution of Intermediate 1-5 (20 mg, 91.05 μmol, 1 eq, HCl salt) and Intermediate 1-6 (72.30 mg, 72.84 μmol, 0.80 eq) in DMF (0.5 mL) was added DIEA (35.30 mg, 273.14 μmol, 47.57 μL, 3 eq). The mixture was stirred at 60° C. for 0.5 hr. The reaction mixture was diluted with H₂O (6 mL) and extracted with EA (5 mL*2). The combined organic layers were washed with NaCl (aq) (5 mL*2), dried over Na₂SO₄, filtered and concentrated under reduced pressure to give a residue. The residue was purified by prep-HPLC (column: Phenomenex luna C18 150*25 mm*10 um; mobile phase: [water (FA)-ACN]; gradient: 12%-42% B over 10 min) and the eluent was concentrated to remove ACN and lyophilized to give I-261 (17.82 mg, 14.82 μmol, 16.28% yield, 98.608% purity, FA salt) as a yellow solid. LCMS (Method D): Retention time: 0.383 min, [M+H]+=1139.6. ¹H NMR (400 MHz, METHANOL-d₄) δ=8.52 (s, 1H), 8.37 (d, J=7.6 Hz, 1H), 8.00-7.92 (m, 1H), 7.91-7.81 (m, 2H), 7.78 (s, 1H), 7.70 (d, J=5.2 Hz, 2H), 7.53-7.42 (m, 3H), 7.40-7.33 (m, 1H), 7.19-7.14 (m, 1H), 4.95 (s, 1H), 4.59-4.51 (m, 2H), 4.47-4.36 (m, 5H), 4.34-4.25 (m, 1H), 4.11-3.97 (m, 5H), 3.91-3.65 (m, 7H), 3.58-3.48 (m, 2H), 3.30-3.13 (m, 4H), 3.05-2.82 (m, 1H), 2.78-2.68 (m, 2H), 2.57 (s, 7H), 2.34-2.22 (m, 2H), 2.11-2.01 (m, 1H), 2.00-1.48 (m, 13H), 1.42-1.38 (m, 3H), 1.36-0.97 (m, 8H). ¹⁹F NMR (377 MHz ETHANOL-d₄) δ=−120.747.

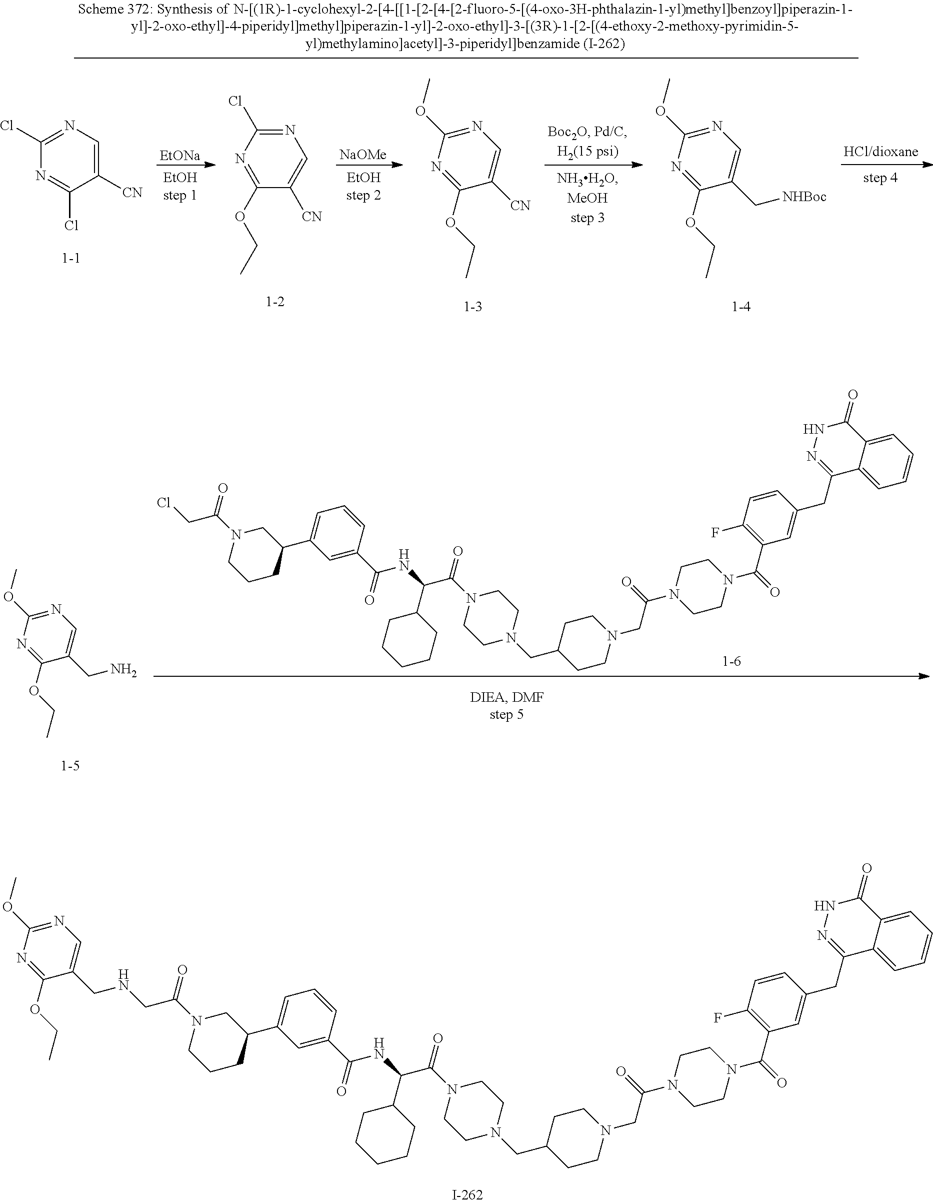

### Step 1: Synthesis of Intermediate 1-2

To a solution of Intermediate 1-1 (1 g, 5.75 mmol, 1 eq) in EtOH (10 mL) was added EtONa (1.96 g, 5.75 mmol, 20% purity, 1 eq). The mixture was stirred at 0° C. for 0.5 hr. DCM (10 mL) and water (8 mL) were added to the reaction mixture and the layers were separated. The aqueous phase was extracted with DCM (10 mL×2). The organic layers were collected and dried under vacuum. The residue was purified by column chromatography (SiO₂, PE/EA=1/0 to 0/1, Rf=0.3, EA/PE=1:20) and the eluent was concentrated in vacuum. Intermediate 1-2 (250 mg, 1.36 mmol, 23.69% yield, 100% purity) was obtained as yellow oil. LCMS (Method D): Rt=0.296 min, [M+H]+=184.0. HPLC: Rt=3.26 min. ¹H NMR (400 MHz, DMSO-d₆) δ=9.14 (s, 1H), 4.54-4.33 (m, 2H), 1.40-1.31 (m, 3H).

### Step 2: Synthesis of Intermediate 1-3

To a solution of Intermediate 1-2 (240 mg, 1.31 mmol, 1 eq) in EtOH (3 mL) was added NaOMe (235.39 mg, 1.31 mmol, 30% purity, 1 eq). The mixture was stirred at 0° C. for 0.5 hr. DCM (5 mL) and water (4 mL) were added and the layers were separated. The aqueous phase was extracted with DCM (4 mL×2). The organic layers were collected and dried under vacuum. The residue was purified by column chromatography (SiO₂, PE/EA=1/0 to 0/1, Rf=0.5, EA/PE=1:10) and the eluent was concentrated in vacuum. Intermediate 1-3 (220 mg, 1.23 mmol, 93.93% yield, 100% purity) was obtained as white oil. LCMS (Method D): Rt=0.288 min, [M+H]⁺=180.1. ¹H NMR (400 MHz, DMSO-d6) δ=8.87 (s, 1H), 4.55-4.44 (m, 2H), 3.98 (s, 3H), 1.40-1.30 (m, 3H).

### Step 3: Synthesis of Intermediate 1-4

To a solution of Intermediate 1-3 (220 mg, 1.23 mmol, 1 eq) in MeOH (3 mL) was added Pd/C (62.86 mg, 59.07 μmol, 10% purity, 4.81e−2 eq), NH₃·H₂O (86.07 mg, 736.71 μmol, 94.58 μL, 30% purity, 0.6 eq) and (Boc)₂O (535.95 mg, 2.46 mmol, 564.16 μL, 2 eq) under N₂ atmosphere. The suspension was degassed and purged with H₂ 3 times. The mixture was stirred under H₂ (15 Psi) at 25° C. for 1 hr. The resulting mixture was filtered and concentrated in vacuo. The residue was purified by column chromatography (SiO₂, PE/EA=1/0 to 0/1, Rf=0.3, EA/PE=1:3) and the eluent was concentrated in vacuum. Intermediate 1-4 (250 mg, 877.67 μmol, 71.48% yield, 99.466% purity) was obtained as a white solid. LCMS (Method D): Rt=0.330 min, [M+H]⁺=284.1. ¹H NMR (400 MHz, DMSO-d₆) δ=8.07 (s, 1H), 7.25-7.11 (m, 1H), 4.40-4.34 (m, 2H), 3.97 (d, J=5.6 Hz, 2H), 3.85 (s, 3H), 1.39-1.36 (m, 9H), 1.34-1.30 (m, 3H).

### Step 4: Synthesis of Intermediate 1-5

A solution of Intermediate 1-4 (100 mg, 352.95 μmol, 1 eq) in HCl/dioxane (2 M, 600.00 μL, 3.40 eq) was stirred at 25° C. for 0.5 hr. The mixture was concentrated in vacuum. The crude product was used in the next step without purification. Intermediate 1-5 (75 mg, crude, HCl salt) was obtained as a white solid. LCMS (Method D): Rt=0.124 min, [M+H]⁺=184.1.

### Step 5: Synthesis of I-262

To a solution of Intermediate 1-5 (15 mg, 68.28 μmol, 1 eq, HCl salt) and Intermediate 1-6 (67.78 mg, 68.28 μmol, 1 eq) in DMF (1 mL) was added DIEA (26.48 mg, 204.85 μmol, 35.68 μL, 3 eq). The resulting mixture was stirred at 60° C. for 1 hr. The crude product was used in the next step without work up. The residue was purified by prep-HPLC (column: CD02-Waters Xbidge BEH C18 150*25*10 um; mobile phase: [water (NH₄HCO₃)-ACN]; gradient: 34%-64% B over 10 min) and the eluent was lyophilized to give product. I-262 (14.5 mg, 12.50 μmol, 18.31% yield, 98.226% purity) was obtained as a white solid. LCMS (Method H): Rt=0.650 min, [M+H]⁺=1139.2. SFC: Rt=8.471 min. ¹H NMR (400 MHz, METHANOL-d₄) δ=8.37 (d, J=8.0 Hz, 1H), 8.17 (d, J=7.2 Hz, 1H), 7.95 (d, J=2.4 Hz, 1H), 7.92-7.65 (m, 4H), 7.55-7.30 (m, 4H), 7.20-7.13 (m, 1H), 4.99-4.90 (m, 2H), 4.69-4.42 (m, 5H), 4.39 (s, 2H), 4.33-4.25 (m, 1H), 3.96 (s, 3H), 3.90-3.84 (m, 1H), 3.79 (d, J=1.2 Hz, 1H), 3.74-3.64 (m, 4H), 3.56 (s, 1H), 3.54-3.40 (m, 3H), 3.27 (d, J=6.8 Hz, 1H), 3.22-3.07 (m, 3H), 2.90-2.61 (m, 4H), 2.52 (d, J=3.6 Hz, 7H), 2.28-2.16 (m, 2H), 2.07-2.00 (m, 1H), 1.99-1.81 (m, 7H), 1.80-1.63 (m, 5H), 1.47-1.39 (m, 3H), 1.38-1.20 (m, 4H), 1.20-0.96 (m, 4H). ¹⁹F NMR (377 MHz, METHANOL-d₄) δ=−120.817.

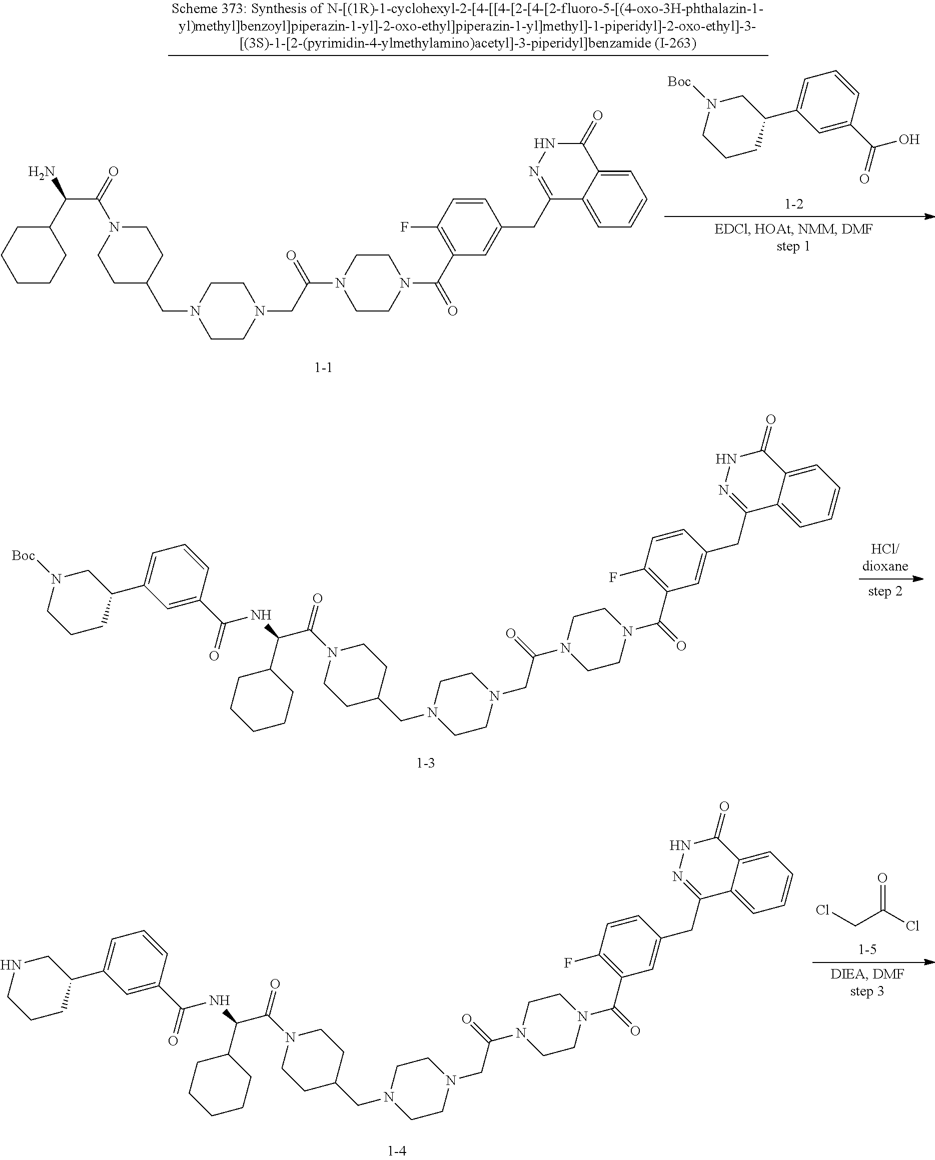

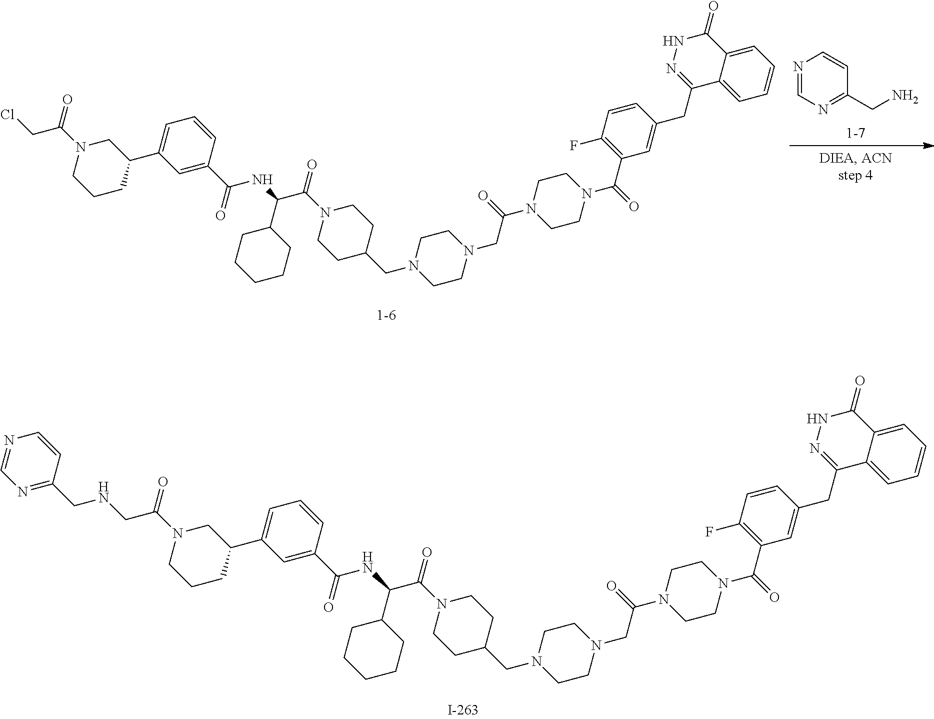

### Step 1: Synthesis of Intermediate 1-3

To a solution of Intermediate 1-2 (230.42 mg, 754.57 μmol, 1.1 eq) in DMF (5 mL) was added EDCI (657.50 mg, 3.43 mmol, 5 eq) and HOAT (186.74 mg, 1.37 mmol, 191.92 μL, 2 eq), NMM (346.92 mg, 3.43 mmol, 377.09 μL, 5 eq), Intermediate 1-1 (500 mg, 685.97 μmol, 1 eq). The mixture was stirred at 25° C. for 2 hr. The reaction mixture was washed with water (3 mL) and extracted with EA (3 mL*3), the combined organic phase was dried by Na₂SO₄ and concentrated under reduced pressure to give a residue. The residue was purified by flash silica gel chromatography (ISCO; 12 g SepaFlashSilica Flash Column, Eluent of 0˜50% Methanol/Dichloromethane @30 mL/min) and the eluent was concentrated to give product. Intermediate 1-3 (340 mg, 308.76 μmol, 45.01% yield, 92.286% purity) was obtained as yellow oil. LCMS (Method D): Retention time=0.435 min, [M+H]+=1016.4. SFC: Retention time: 1.986 min.

### Step 2: Synthesis of Intermediate 1-4

A solution of Intermediate 1-3 (340 mg, 334.56 mol, 1 eq) in HCl/dioxane (2 M, 3.4 mL, 20.32 eq) was stirred at 25° C. for 1 hr. The mixture was concentrated under reduced pressure to give a residue. The crude product was used in the next step without further purification. Intermediate 1-4 (350 mg, crude, HCl salt) was obtained as an off-white solid. LCMS (Method D): Retention time=0.321 min, [M+H]+=916.4.

### Step 3: Synthesis of Intermediate 1-6

To a mixture of Intermediate 1-4 (340 mg, 356.92 μmol, 1 eq, HCl salt) and Intermediate 1-5 (48.37 mg, 428.30 μmol, 34.11 μL, 1.2 eq) in DMF (2 mL) was added DIEA (92.26 mg, 713.84 μmol, 124.34 μL, 2 eq). The mixture was stirred at 25° C. for 1 hr. The reaction mixture was filtered to give the filtrate. The crude was purified by prep-HPLC (column: Phenomenex luna C18 150*25 mm*10 um; mobile phase: [water (FA)-ACN]; gradient: 18%-48% B over 10 min) the eluent was concentrated to remove ACN and lyophilized to give product. Intermediate 1-6 (300 mg, 282.77 μmol, 79.22% yield, 93.559% purity) was obtained as a white solid. LCMS (Method D): Retention time=0.393 min, [M+H]+=993.0. SFC: Retention time: 2.198 min.

### Step 4: Synthesis of I-263

To a mixture of Intermediate 1-6 (100 mg, 100.74 μmol, 1 eq) in ACN (1 mL) was added DIEA (39.06 mg, 302.23 μmol, 52.64 μL, 3 eq) and Intermediate 1-7 (13.19 mg, 120.89 μmol, 1.2 eq). The mixture was stirred at 60° C. for 2 hr. The reaction mixture was filtered to give the filtrate. The crude was purified by prep-HPLC (column: Phenomenex luna C18 150*25 mm*10 um; mobile phase: [water (FA)-ACN]; gradient: 5%-35% B over 8 min) and the eluent was concentrated to remove ACN and lyophilized to give product. I-263 (17 mg, 15.05 μmol, 14.94% yield, 98.386% purity, FA salt) was obtained as a yellow solid. LCMS (Method D): Retention time=0.324 min, [M+H]+=1065.6. SFC: Retention time: 1.495 min. ¹H NMR (400 MHz, METHANOL-d₄) δ=9.11 (d, J=11.2 Hz, 1H), 8.77-8.68 (m, 1H), 8.48 (s, 1H), 8.37 (d, J=7.6 Hz, 1H), 7.98-7.92 (m, 1H), 7.90-7.81 (m, 2H), 7.78-7.68 (m, 2H), 7.61-7.54 (m, 1H), 7.49 (d, J=6.0 Hz, 2H), 7.46-7.41 (m, 1H), 7.40-7.34 (m, 1H), 7.16 (t, J=8.8 Hz, 1H), 4.97-4.91 (m, 1H), 4.60-4.50 (m, 2H), 4.39 (s, 2H), 4.34-4.27 (m, 1H), 4.04 (d, J=12.0 Hz, 2H), 3.86 (d, J=12.8 Hz, 1H), 3.81-3.62 (m, 6H), 3.56-3.48 (m, 2H), 3.39-3.32 (m, 3H), 3.29-3.08 (m, 3H), 2.89-2.80 (m, 1H), 2.79-2.69 (m, 3H), 2.63 (d, J=13.6 Hz, 7H), 2.41-2.29 (m, 2H), 2.09-2.02 (m, 1H), 1.96-1.83 (m, 6H), 1.78 (d, J=8.0 Hz, 3H), 1.70-1.60 (m, 3H), 1.34-1.20 (m, 4H), 1.16-1.05 (m, 3H). ¹⁹F NMR (377 MHz, METHANOL-d₄) δ=−120.785.

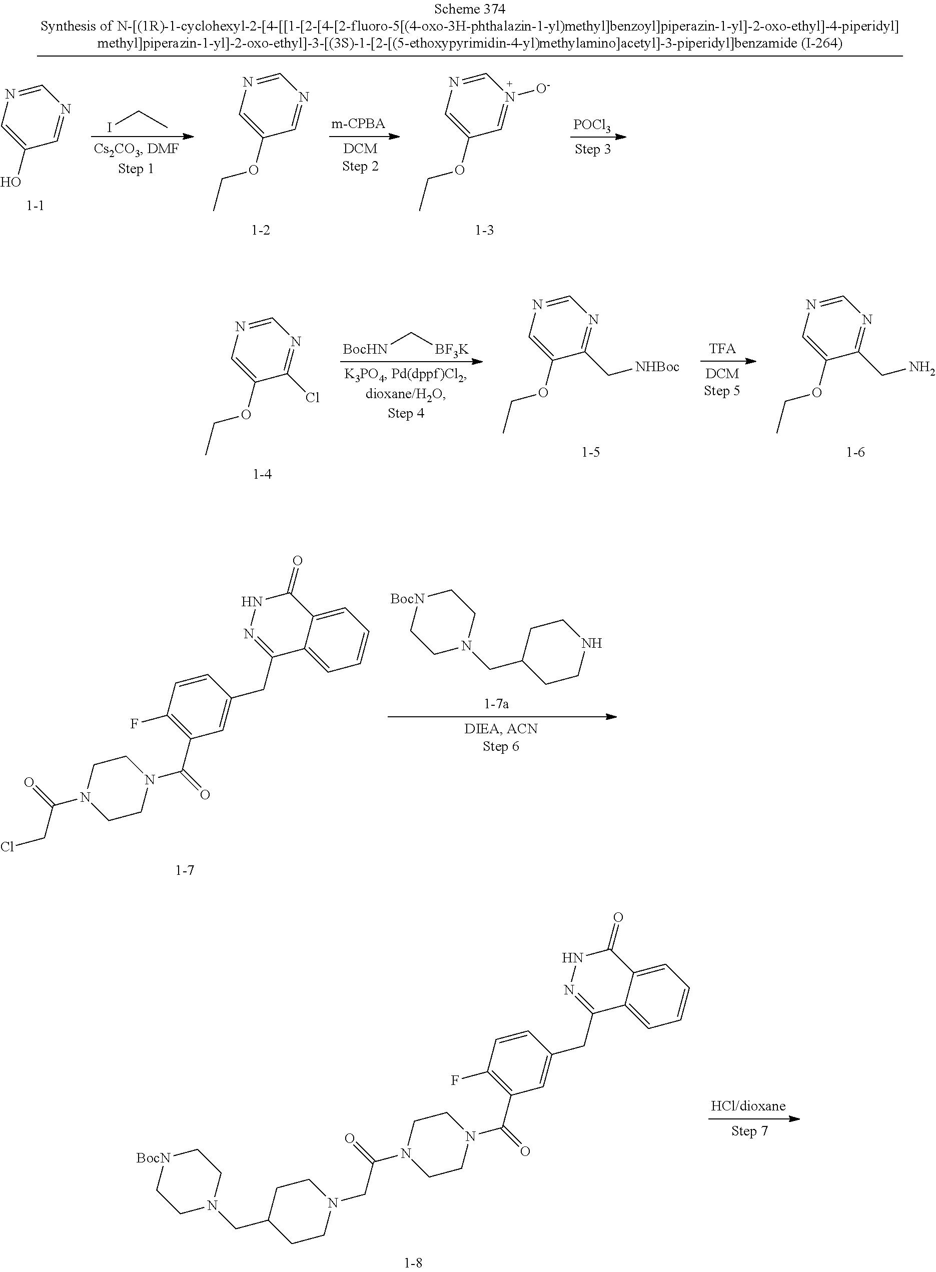

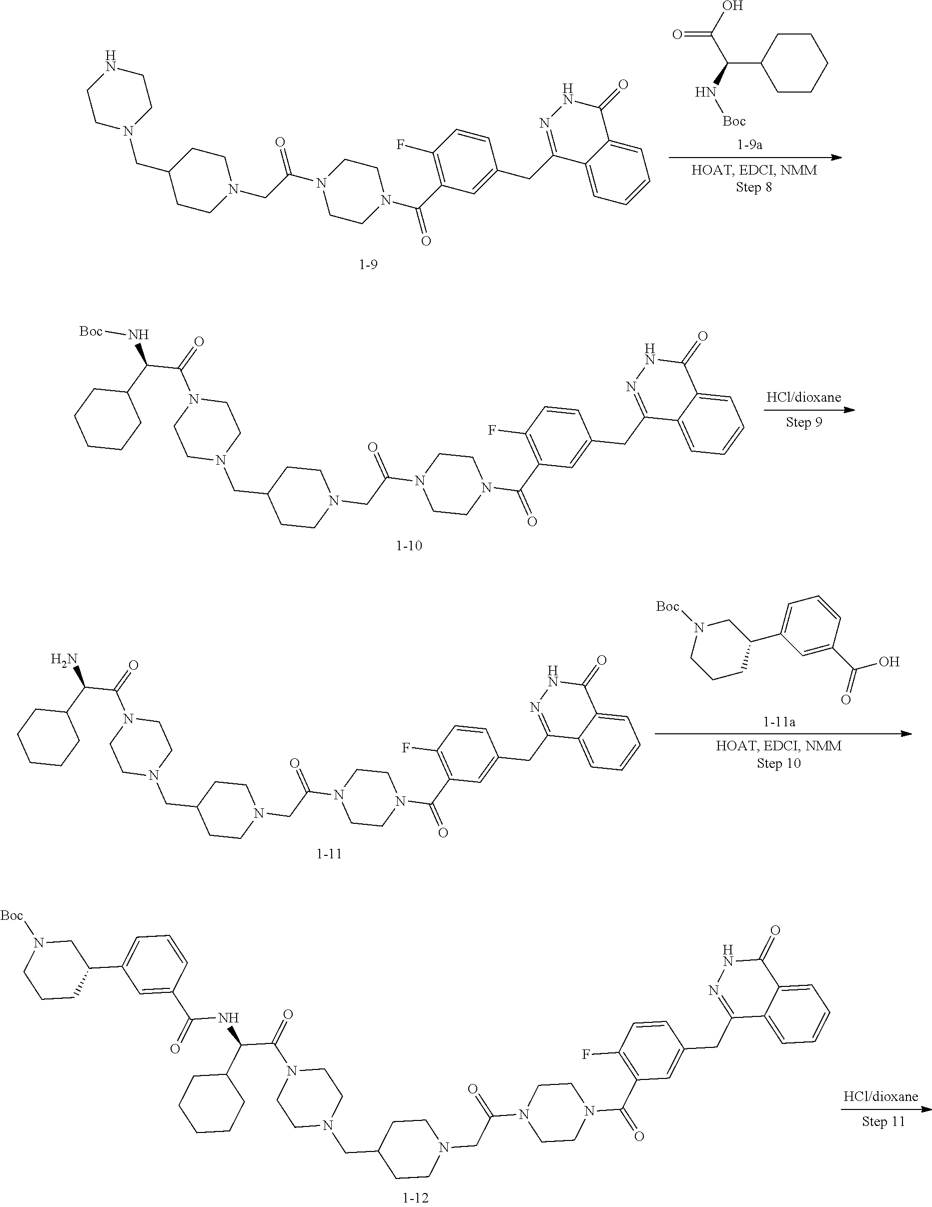

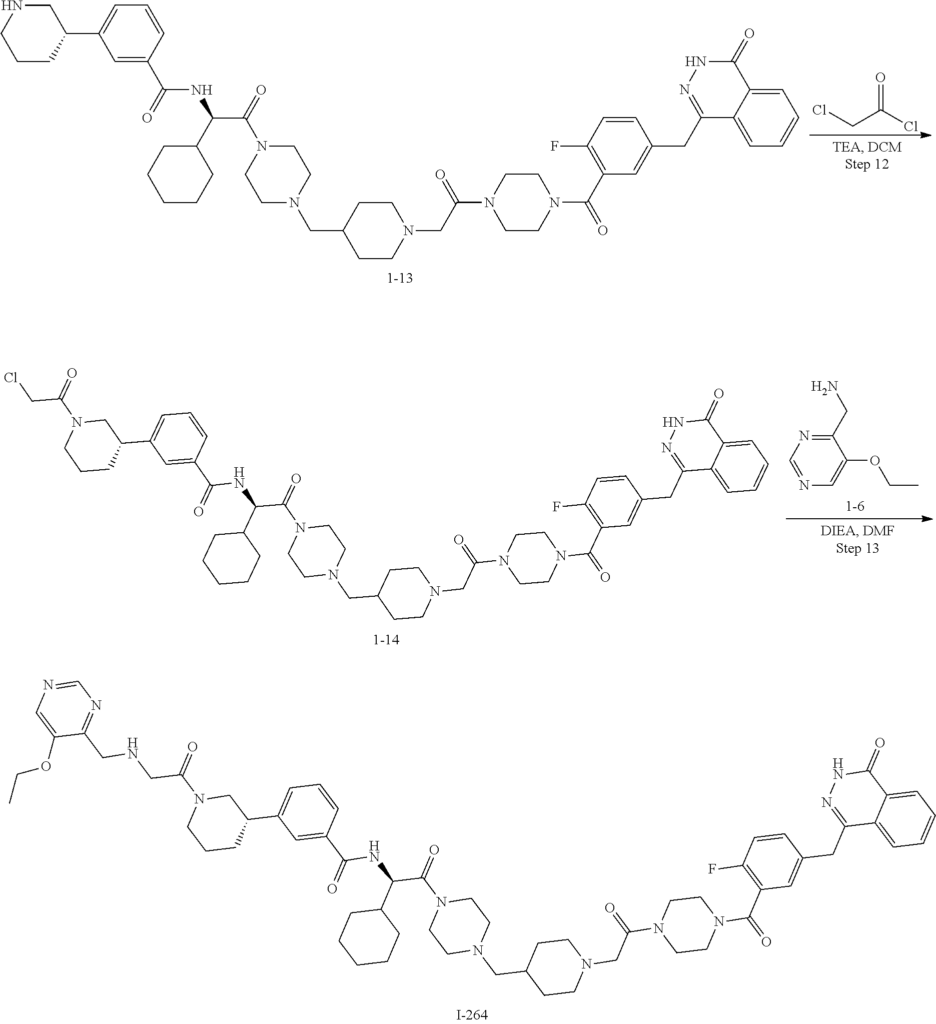

### Step 1: Synthesis of Intermediate 1-2.

To a mixture of Intermediate 1-1 (1.8 g, 18.73 mmol, 1 eq) and EtI (3.51 g, 22.48 mmol, 1.80 mL, 1.2 eq) in DMF (20 mL) was added Cs₂CO₃ (18.31 g, 56.20 mmol, 3 eq), then the mixture was stirred at 60° C. for 1 hour. The reaction mixture was diluted with water (60 mL) and extracted with EA (10 mL*3). The combined organic layers were concentrated under reduced pressure to give a residue. The residue was purified by flash silica gel chromatography (ISCO; 40 g SepaFlash Silica Flash Column, Eluent of 0˜50% Ethyl acetate/Petroleum ether gradient @80 mL/min). The eluent was concentrated under reduced pressure to give Intermediate 1-2 (2 g, 16.11 mmol, 86.00% yield) as a white solid. LCMS (Method D): Retention time: 0.185 min, [M+H]+=125.1. ¹H NMR (400 MHz, CHLOROFORM-d) δ=8.84 (s, 1H), 8.40 (s, 2H), 4.20-4.09 (m, 2H), 1.48 (t, J=6.8 Hz, 4H).

### Step 2: Synthesis of Intermediate 1-3.

To a mixture of Intermediate 1-2 (1.8 g, 14.50 mmol, 1 eq) in DCM (30 mL) was added m-CPBA (3.53 g, 17.40 mmol, 85% purity, 1.2 eq) at 0° C., then the mixture was stirred at 20° C. for 1 hour. The reaction mixture was quenched by addition of a saturated Na₂SO₃ solution (50 mL) at 0° C., and then it was stirred for 0.5 hr. Then the mixture was diluted with H₂O (20 mL) and extracted with EA (5 mL*3). The combined organic layers were washed with a 10% Na₂SO₃ solution (5 mL*3) and brine (5 mL*3), dried over Na₂SO₄, filtered and concentrated under reduced pressure to give a residue. The residue was purified by flash silica gel chromatography (ISCO; 20 g SepaFlash Silica Flash Column, Eluent of 0˜100% Ethyl acetate/Petroleum ether gradient @60 mL/min). The eluent was concentrated under reduced pressure to give Intermediate 1-3 (1.5 g, 10.70 mmol, 73.82% yield, 100% purity) as a white solid. LCMS (Method A): Retention time: 0.130 min, [M+H]+=141.1.

### Step 3: Synthesis of Intermediate 1-4.

A mixture of Intermediate 1-3 (1.5 g, 10.70 mmol, 1 eq) in POCl₃ (10 mL) was stirred at 100° C. for 0.5 hour. The mixture was concentrated under reduced pressure to give a residue. Then the residue was quenched by H₂O (30 ml) at 20° C., and adjusted to pH 7-8 with NaHCO₃, and the mixture was extracted with EA (10 ml*3), and the combined organic phase was dried with anhydrous sodium sulfate, filtered and the filtrate was concentrated to give product. The residue was purified by flash silica gel chromatography (ISCO; 40 g SepaFlash Silica Flash Column, Eluent of 0˜60% Ethyl acetate/Petroleum ether gradient @80 mL/min). The eluent was concentrated under reduced pressure to give Intermediate 1-4 (1 g, 5.86 mmol, 54.79% yield, 93% purity) as a white solid. LCMS (Method D): Retention time: 0.264 min, [M+H]+=159.1. ¹H NMR (400 MHz, DMSO-d₆) δ=8.65 (s, 1H), 8.63 (s, 1H), 4.30 (q, J=7.2 Hz, 2H), 1.41-1.37 (m, 3H).

### Step 4: Synthesis of Intermediate 1-5.

To a mixture of Intermediate 1-4 (0.9 g, 5.68 mmol, 1 eq) and potassium;(tert-butoxycarbonylamino)methyl-trifluoro-boranuide (2.42 g, 10.22 mmol, 1.8 eq) in dioxane (20 mL) and H₂O (4 mL) was added K₃PO₄ (3.61 g, 17.03 mmol, 3 eq), Pd(dppf)Cl₂ (415.26 mg, 567.52 μmol, 0.1 eq), then the mixture was stirred at 100° C. for 16 hours under N₂. The reaction mixture was diluted with water (50 mL) and extracted with EA (10 mL*2). The combined organic layers were concentrated under reduced pressure to give a residue. The residue was purified by flash silica gel chromatography (ISCO; 20 g SepaFlash Silica Flash Column, Eluent of 0˜40% Ethyl acetate/Petroleum ether gradient @60 mL/min). The eluent was concentrated under reduced pressure to give Intermediate 1-5 (350 mg, 1.34 mmol, 23.62% yield, 97% purity) as a white solid. LCMS (Method D): Retention time: 0.343 min, [M+H]+=198.1. ¹H NMR (400 MHz, DMSO-d₆) δ=8.71 (s, 1H), 8.46 (s, 1H), 7.02 (t, J=5.2 Hz, 1H), 4.24 (d, J=6.0 Hz, 2H), 4.22-4.17 (m, 3H), 1.39 (s, 9H), 1.37-1.35 (m, 3H).

### Step 5: Synthesis of Intermediate 1-6.

To a mixture of Intermediate 1-5 (100 mg, 394.79 μmol, 1 eq) in DCM (1 mL) was added TFA (0.2 mL) and then the mixture was stirred at 20° C. for 1 hour. The reaction mixture was concentrated under reduced pressure to give Intermediate 1-6 (120 mg, crude, TFA salt) as a white solid. LCMS: Retention time: 0.078 min, [M+H]+=154.1.

### Step 6: Synthesis of Intermediate 1-8.

To a mixture of Intermediate 1-7a (5.12 g, 18.06 mmol, 1 eq) and Intermediate 1-7 (8 g, 18.06 mmol, 1 eq) in ACN (100 mL) was added DIEA (9.34 g, 72.26 mmol, 12.59 mL, 4 eq), then the mixture was stirred at 20° C. for 1 hour. The reaction mixture was diluted with water (200 mL) and extracted with EA (50 mL*2). The combined organic layers were concentrated under reduced pressure to give Intermediate 1-8 (11 g, 15.95 mmol, 88.28% yield) as a white solid. LCMS (Method D): Retention time: 0.326 min, [M+H]+=690.6. ¹H NMR (400 MHz, DMSO-d₆) δ=12.59 (s, 1H), 8.26 (d, J=7.6 Hz, 1H), 7.99-7.93 (m, 1H), 7.90-7.87 (m, 1H), 7.86-7.80 (m, 1H), 7.48-7.30 (m, 2H), 7.23 (t, J=8.8 Hz, 1H), 4.33 (s, 2H), 3.90 (d, J=11.4 Hz, 2H), 3.61 (s, 2H), 3.50 (s, 2H), 3.18 (s, 2H), 2.83 (t, J=4.8 Hz, 1H), 2.67 (d, J=1.6 Hz, 2H), 2.35 (s, 8H), 2.11-2.05 (m, 2H), 1.63 (s, 4H), 1.38 (s, 9H), 1.02-0.89 (m, 4H).

### Step 7: Synthesis of Intermediate 1-9.

A mixture of Intermediate 1-8 (10 g, 14.50 mmol, 1 eq) in DCM (70 mL) was added HCl/dioxane (2 M, 36.24 mL, 5 eq) and then the mixture was stirred at 20° C. for 5 min. The reaction mixture was concentrated under reduced pressure to give Intermediate 1-9 (10 g, crude, HCl salt) as a white solid. LCMS (Method D): Retention time: 0.245 min, [M+H]+=590.2.

### Step 8: Synthesis of Intermediate 1-10.

To a mixture of Intermediate 1-9 (8.00 g, 12.78 mmol, 1 eq, HCl salt) and Intermediate 1-9a (6.58 g, 25.55 mmol, 2 eq) in DMF (100 mL) was added HOAt (1.74 g, 12.78 mmol, 1.79 mL, 1 eq), EDCI (7.35 g, 38.33 mmol, 3 eq) and NMM (6.46 g, 63.88 mmol, 7.02 mL, 5 eq), then the mixture was stirred at 25° C. for 1 hour. The reaction mixture was diluted with water (200 mL) and extracted with EA (50 mL*2). The combined organic layers were concentrated under reduced pressure to give a residue. The residue was purified by flash silica gel chromatography ISCO; 120 g SepaFlash Silica Flash Column, Eluent of 0˜10% DCM/MeOH gradient @100 mL/min). The eluent was concentrated under reduced pressure to give Intermediate 1-10 (8 g, 9.46 mmol, 74.02% yield, 98% purity) as a white solid. LCMS (Method D): Retention time: 0.372 min, [M+H]+=829.5. SFC: Rt=1.214 min.

### Step 9: Synthesis of Intermediate 1-11.

To a mixture of t Intermediate 1-10 (7.00 g, 8.44 mmol, 1 eq) in DCM (40 mL) was added HCl/dioxane (2 M, 42.22 mL, 10 eq), then the mixture was stirred at 20° C. for 10 min. The reaction mixture was concentrated under reduced pressure to give Intermediate 1-11 (11 g, crude, HCl salt) as a white solid. LCMS (Method D): Retention time: 0.277 min, [M+H]+=729.4.

### Step 10: Synthesis of Intermediate 1-12.

To a mixture of Intermediate 1-11 (4.51 g, 5.89 mmol, 1.5 eq, HCl salt) and Intermediate 1-11a (1.2 g, 3.93 mmol, 1 eq) in DMF (12 mL) was added HOAt (534.87 mg, 3.93 mmol, 549.72 μL, 1 eq), EDCI (2.26 g, 11.79 mmol, 3 eq), NMM (1.99 g, 19.65 mmol, 2.16 mL, 5 eq), then the mixture was stirred at 20° C. for 1 hour. The reaction mixture was quenched with water (200 mL) and extracted with EA (50 mL*3). The combined organic layers were concentrated under reduced pressure to give a residue. The residue was purified by flash silica gel chromatography (ISCO; 80 g SepaFlash Silica Flash Column, Eluent of 0˜10% DCM/MeOH @80 mL/min). The eluent was concentrated under reduced pressure to give Intermediate 1-12 (3 g, 2.62 mmol, 66.63% yield, 88.7% purity) as a white solid. LCMS (Method D): Retention time: 0.401 min, [M+H]+=1016.4. ¹H NMR (400 MHz, CHLOROFORM-d) δ=10.19 (d, J=6.8 Hz, 1H), 8.53-8.42 (m, 1H), 8.02 (s, 1H), 7.81-7.74 (m, 2H), 7.73-7.66 (m, 2H), 7.37 (br s, 2H), 7.17-6.94 (m, 2H), 5.31 (s, 1H), 5.15-5.04 (m, 1H), 4.60-4.59 (m, 1H), 4.29 (s, 2H), 4.25-4.03 (m, 3H), 3.88-3.66 (m, 4H), 3.65-3.54 (m, 2H), 3.39-3.07 (m, 5H), 2.81-2.60 (m, 4H), 2.57-2.37 (m, 7H), 2.25-2.13 (m, 2H), 2.08-1.94 (m, 2H), 1.76 (br d, J=12.0 Hz, 8H), 1.67 (s, 9H), 1.47 (s, 6H), 1.15 (d, J=12.8 Hz, 4H).

### Step 11: Synthesis of Intermediate 1-13.

To a mixture of Intermediate 1-12 (2.8 g, 2.76 mmol, 1 eq) in DCM (10 mL) was added HCl/dioxane (2 M, 13.78 mL, 10 eq), then the mixture was stirred at 20° C. for 10 min. The reaction mixture was concentrated under reduced pressure to give Intermediate 1-13 (3 g, crude, HCl salt) as a white solid. LCMS (Method D): Retention time: 0.288 min, [M+H]+=916.4.

### Step 12: Synthesis of Intermediate 1-14.

To a mixture of Intermediate 1-13 (3 g, 3.15 mmol, 1 eq, HCl salt) and 2-chloroacetyl chloride (426.83 mg, 3.78 mmol, 301.01 μL, 1.2 eq) in DCM (40 mL) was added TEA (1.59 g, 15.75 mmol, 2.19 mL, 5 eq), then the mixture was stirred at 0° C. for 10 min. The reaction mixture was diluted with water (100 mL) and extracted with DCM (80 mL*2). The combined organic layers were concentrated under reduced pressure to give a residue. The crude product was purified by reversed-phase HPLC (0.1% FA condition). The eluent was concentrated under reduced pressure to remove MeCN and then lyophilized to give Intermediate 1-14 (2.3 g, 2.06 mmol, 65.48% yield, 89% purity) as a white solid. LCMS (Method D): Retention time: 0.428 min, [M+H]+=992.5. SFC: Rt=0.827 min. ¹H NMR (400 MHz, DMSO-d₆) δ=12.59 (s, 1H), 8.51-8.38 (m, 1H), 8.26 (d, J=7.6 Hz, 1H), 7.99-7.95 (m, 1H), 7.92-7.72 (m, 4H), 7.46-7.34 (m, 3H), 7.24 (t, J=8.8 Hz, 1H), 4.83-4.73 (m, 1H), 4.44-4.31 (m, 5H), 4.24-4.13 (m, 1H), 3.94-3.79 (m, 1H), 3.61-3.01 (m, 22H), 2.87-2.72 (m, 1H), 2.69-2.56 (m, 2H), 2.25-2.08 (m, 2H), 1.98-1.84 (m, 2H), 1.81-1.58 (m, 10H), 1.18-0.84 (m, 7H). ¹⁹F NMR (376 MHz, DMSO-d₆) δ=−119.77.

### Step 13: Synthesis of I-264

To a mixture of Intermediate 1-14 (74.30 mg, 74.85 μmol, 0.5 eq) and Intermediate 1-6 (40 mg, 149.70 μmol, 1 eq, TFA salt) in DMF (1 mL) was added DIEA (96.74 mg, 748.49 μmol, 130.37 μL, 5 eq), then the mixture was stirred at 40° C. for 16 hours. The reaction mixture was quenched with water (60 mL) and extracted with EA (10 mL*3). The combined organic layers were concentrated under reduced pressure to give a residue. The crude product was purified by reversed-phase HPLC (0.1% TFA condition). The eluent was concentrated under reduced pressure to remove MeCN and then lyophilized to give I-264 (2.94 mg, 2.37 μmol, 1.58% yield, 98.5% purity, TFA salt) as a yellow solid. LCMS (Method D): Retention time: 0.345 min, [M+H]+=1109.5. SFC: Rt=3.779 min. ¹H NMR (400 MHz, METHANOL-d₄) δ=8.82 (d, J=4.0 Hz, 1H), 8.54 (d, J=6.8 Hz, 1H), 8.37 (d, J=7.6 Hz, 1H), 7.99-7.92 (m, 1H), 7.92-7.81 (m, 2H), 7.80-7.67 (m, 2H), 7.53-7.42 (m, 3H), 7.39-7.34 (m, 1H), 7.20-7.15 (m, 1H), 4.95-4.91 (m, 1H), 4.65-4.55 (m, 2H), 4.53-4.47 (m, 2H), 4.42-4.25 (m, 7H), 3.84-3.61 (m, 7H), 3.55-3.44 (m, 2H), 3.35 (s, 3H), 3.26-3.16 (m, 4H), 3.04 (d, J=4.8 Hz, 3H), 2.97-2.66 (m, 6H), 2.18-2.05 (m, 2H), 1.96-1.63 (m, 11H), 1.52-1.45 (m, 3H), 1.41-1.27 (m, 4H), 1.24-1.05 (m, 4H). ¹⁹F NMR (376 MHz, METHANOL-d₄) δ=−77.099, −120.66.

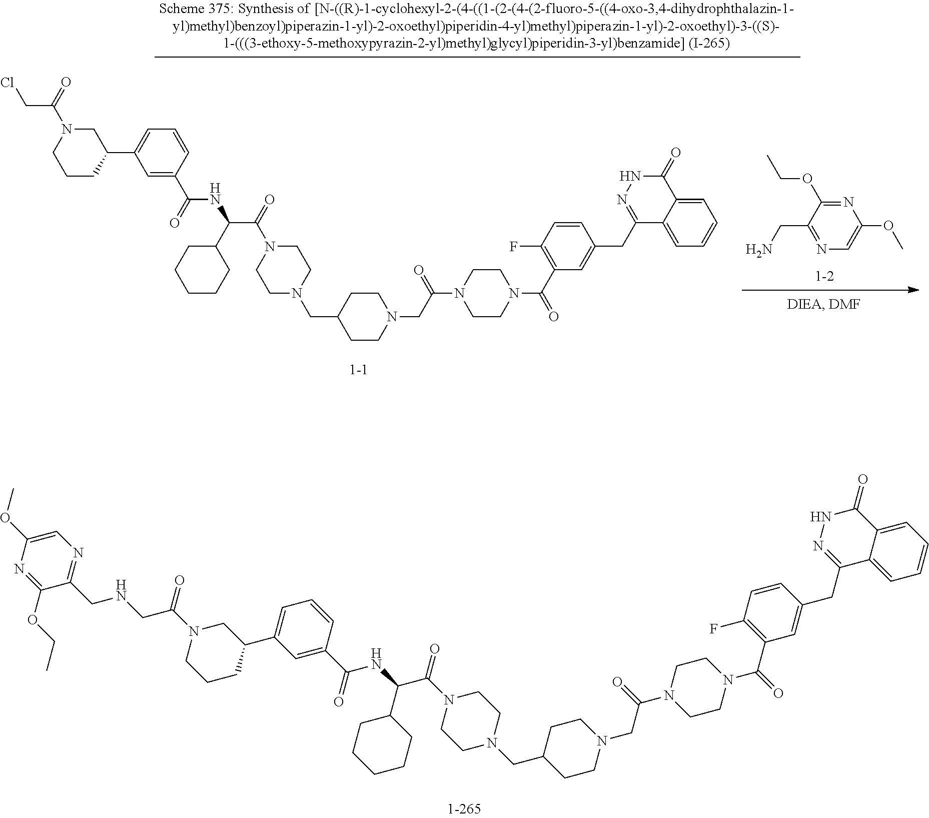 [SMILES: O=C(N[C@@H](C(=O)N1CCN(CC2CCN(CC(=O)N3CCN(C(=O)c4cc(Cc5n[nH]c(=O)c6ccccc56)ccc4F)CC3)CC2)CC1)C1CCCCC1)c1cccc([C@@H]2CCCN(C(=O)CCl)C2)c1 ; CCOc1nc(OC)cnc1CN ; CCOc1nc(OC)cnc1CNCC(=O)N1CCC[C@@H](c2cccc(C(=O)N[C@@H](C(=O)N3CCN(CC4CCN(CC(=O)N5CCN(C(=O)c6cc(Cc7n[nH]c(=O)c8ccccc78)ccc6F)CC5)CC4)CC3)C3CCCCC3)c2)C1]

To a solution of Intermediate 1-2 (20 mg, 91.05 μmol, 1 eq, HCl salt) and Intermediate 1-1 (72.30 mg, 72.84 μmol, 0.8 eq) in DMF (0.5 mL) was added DIEA (35.30 mg, 273.14 μmol, 47.58 μL, 3 eq). The mixture was stirred at 60° C. for 0.5 hr. The reaction mixture was diluted with H₂O (6 mL) and extracted with EA (5 mL*2). The combined organic layers were washed with NaCl (aq) (5 mL*2), dried over Na₂SO₄, filtered and concentrated under reduced pressure to give a residue. The residue was purified by prep-HPLC (column: Phenomenex luna C18 150*25 mm*10 um; mobile phase: [water (FA)-ACN]; gradient: 13%-43% B over 10 min). The eluent was concentrated to remove ACN and lyophilized to give product. I-265 (11.61 mg, 10.00 μmol, 10.99% yield, 98.179% purity) was obtained as a white solid. LCMS (Method D): Retention time: 0.395 min, [M+H]⁺=1139.7. SFC: Retention time: 3.232 min. ¹H NMR (400 MHz, METHANOL-d₄) δ=8.37 (d, J=7.6 Hz, 1H), 7.99-7.92 (m, 1H), 7.91-7.81 (m, 2H), 7.79-7.66 (m, 3H), 7.53-7.32 (m, 4H), 7.19-7.14 (m, 1H), 4.99-4.91 (m, 1H), 4.63-4.47 (m, 3H), 4.43-4.37 (m, 4H), 4.34-4.26 (m, 1H), 4.07-3.93 (m, 5H), 3.85-3.65 (m, 7H), 3.57-3.49 (m, 2H), 3.27-3.08 (m, 4H), 2.89-2.79 (m, 1H), 2.77-2.69 (m, 2H), 2.63-2.37 (m, 8H), 2.31-2.20 (m, 2H), 2.10-2.02 (m, 1H), 1.94-1.74 (m, 9H), 1.72-1.58 (m, 3H), 1.42-1.36 (m, 3H), 1.33-1.01 (m, 8H). ¹⁹F NMR (377 MHz, METHANOL-d₄) δ=−120.732.

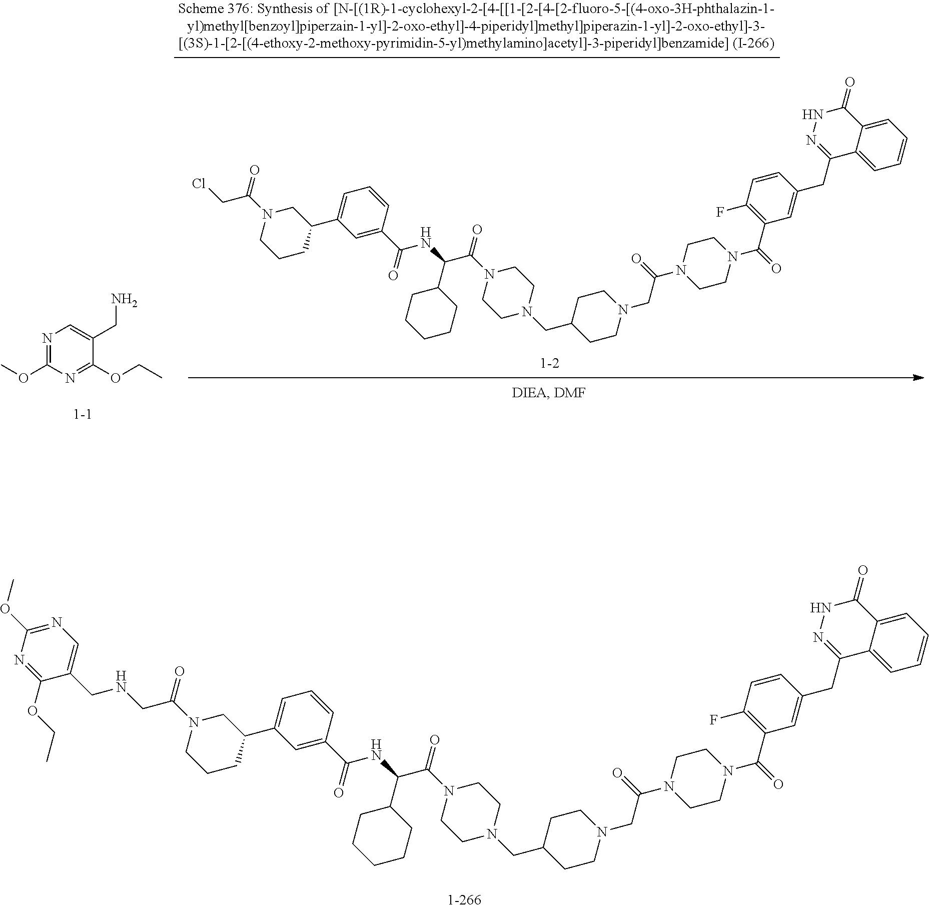 [SMILES: CCOc1nc(OC)ncc1CN ; O=C(N[C@@H](C(=O)N1CCN(CC2CCN(CC(=O)N3CCN(C(=O)c4cc(Cc5n[nH]c(=O)c6ccccc56)ccc4F)CC3)CC2)CC1)C1CCCCC1)c1cccc([C@@H]2CCCN(C(=O)CCl)C2)c1 ; CCOc1nc(OC)ncc1CNCC(=O)N1CCC[C@@H](c2cccc(C(=O)N[C@@H](C(=O)N3CCN(CC4CCN(CC(=O)N5CCN(C(=O)c6cc(Cc7n[nH]c(=O)c8ccccc78)ccc6F)CC5)CC4)CC3)C3CCCCC3)c2)C1]

To a solution of intermediate 1-1 (10 mg, 45.52 μmol, 1 eq, HCl salt) and intermediate 1-2 (45.19 mg, 45.52 μmol, 1 eq) in DMF (1 mL) was added DIEA (17.65 mg, 136.57 μmol, 23.79 μL, 3 eq). The resulting mixture was stirred at 60° C. for 1 hr. The crude product was used in the next step without work up. The residue was purified by prep-HPLC (column: CD02-Waters Xbidge BEH C18 150*25*10 um; mobile phase: [water (NH₄HCO₃)-ACN]; gradient: 34%-64% B over 10 min) and the eluent was lyophilized to give product. I-266 (6.5 mg, 5.46 μmol, 12.00% yield, 95.775% purity) was obtained as a white solid. LCMS (Method D): Rt=0.336 min, [M+H]⁺=1139.5. SFC: Rt=6.962 min. ¹H NMR (400 MHz, METHANOL-d₄) δ=8.39-8.30 (m, 2H), 8.02-7.80 (m, 3H), 7.79-7.68 (m, 2H), 7.57-7.40 (m, 3H), 7.40-7.31 (m, 1H), 7.22-7.10 (m, 1H), 4.97-4.88 (m, 2H), 4.59 (s, 5H), 4.39 (s, 3H), 4.22-4.15 (m, 3H), 4.01 (d, J=4.0 Hz, 3H), 3.82-3.68 (m, 5H), 3.65-3.42 (m, 4H), 3.25-3.14 (m, 6H), 3.12-2.97 (m, 4H), 2.92-2.82 (m, 3H), 2.81-2.71 (m, 2H), 2.15-2.03 (m, 2H), 1.97-1.84 (m, 5H), 1.82-1.56 (m, 6H), 1.52-1.39 (m, 3H), 1.39-1.23 (m, 4H), 1.23-0.99 (m, 4H). ¹⁹F NMR (400 MHz, METHAN OL-d₄) δ=−77.301.

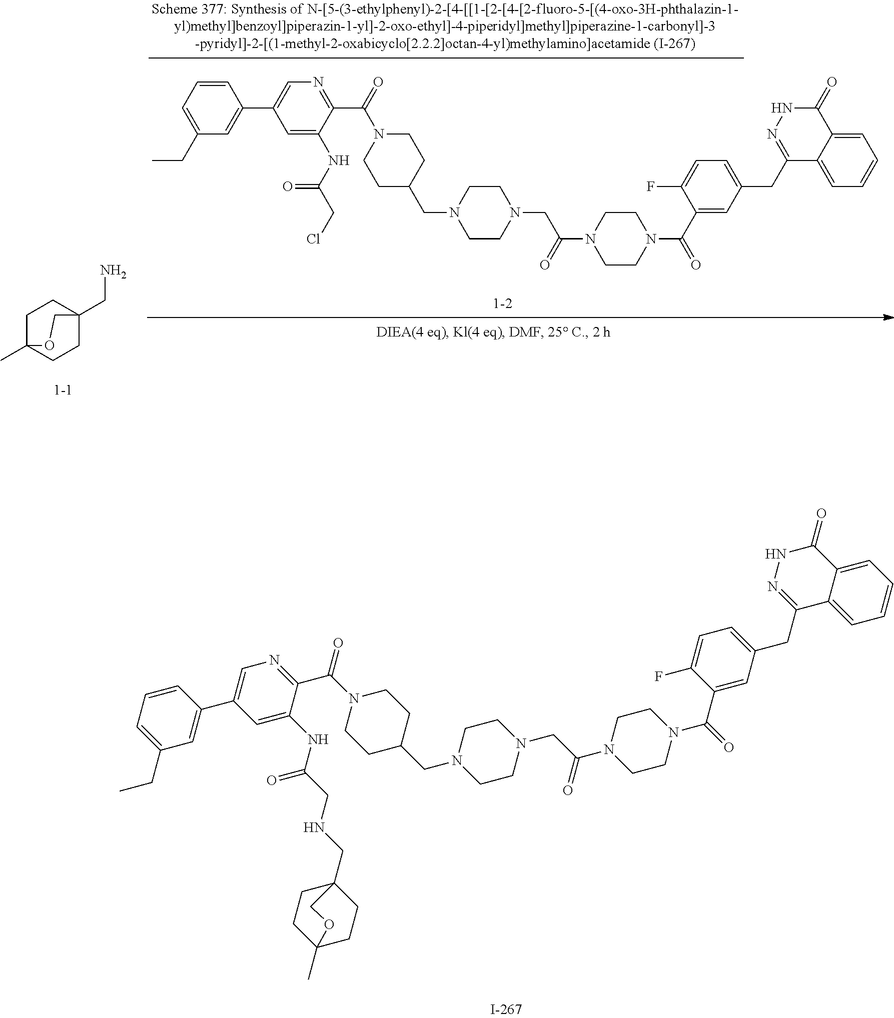 [SMILES: CCc1cccc(-c2cnc(C(=O)N3CCC(CN4CCN(CC(=O)N5CCN(C(=O)c6cc(Cc7n[nH]c(=O)c8ccccc78)ccc6F)CC5)CC4)CC3)c(NC(=O)CCl)c2)c1 ; CC12CCC(CN)(CC1)CO2 ; CCc1cccc(-c2cnc(C(=O)N3CCC(CN4CCN(CC(=O)N5CCN(C(=O)c6cc(Cc7n[nH]c(=O)c8ccccc78)ccc6F)CC5)CC4)CC3)c(NC(=O)CNCC34CCC(C)(CC3)OC4)c2)c1]

To a solution of Intermediate 1-1 (14.64 mg, 94.34 μmol, 1.2 eq) and Intermediate 1-2 (70.00 mg, 78.61 μmol, 1 eq) in DMF (1 mL) was added KI (52.20 mg, 314.45 μmol, 4 eq) and DIEA (40.64 mg, 314.45 μmol, 54.77 μL, 4 eq). The mixture was stirred at 25° C. for 2 hrs. The reaction mixture was filtered and purified by prep-HPLC (column: Waters Xbridge 150*25 mm*5 um; mobile phase: [water (NH₄HCO₃)-ACN]; gradient: 35%-65% B over 15 min). The eluent was concentrated under reduced pressure to remove ACN, then lyophilized to afford I-267 (10 mg, 9.91 μmol, 12.60% yield, 100% purity) as a white solid. LCMS (Method G): Rt=0.668 min, [M+H]⁺=1009.7. ¹H NMR (400 MHz, METHANOL-d₄) δ=8.90 (d, J=2.0 Hz, 1H), 8.59 (d, J=2.0 Hz, 1H), 8.37 (d, J=7.6 Hz, 1H), 7.98-7.93 (m, 1H), 7.91-7.80 (m, 2H), 7.57-7.46 (m, 3H), 7.44-7.42 (m, 1H), 7.40-7.34 (m, 1H), 7.31 (d, J=7.6 Hz, 1H), 7.20-7.13 (m, 1H), 4.77-4.68 (m, 2H), 4.61-4.56 (m, 1H), 4.39 (s, 2H), 3.86-3.64 (m, 7H), 3.59-3.48 (m, 2H), 3.37-3.34 (m, 2H), 3.27 (d, J=1.8 Hz, 1H), 3.22-3.17 (m, 1H), 3.16-3.10 (m, 1H), 2.99-2.90 (m, 1H), 2.75-2.73 (m, 2H), 2.60-2.35 (m, 9H), 2.31-2.21 (m, 2H), 2.01-1.88 (m, 2H), 1.85-1.62 (m, 9H), 1.31-1.27 (m, 6H), 1.09 (s, 3H).

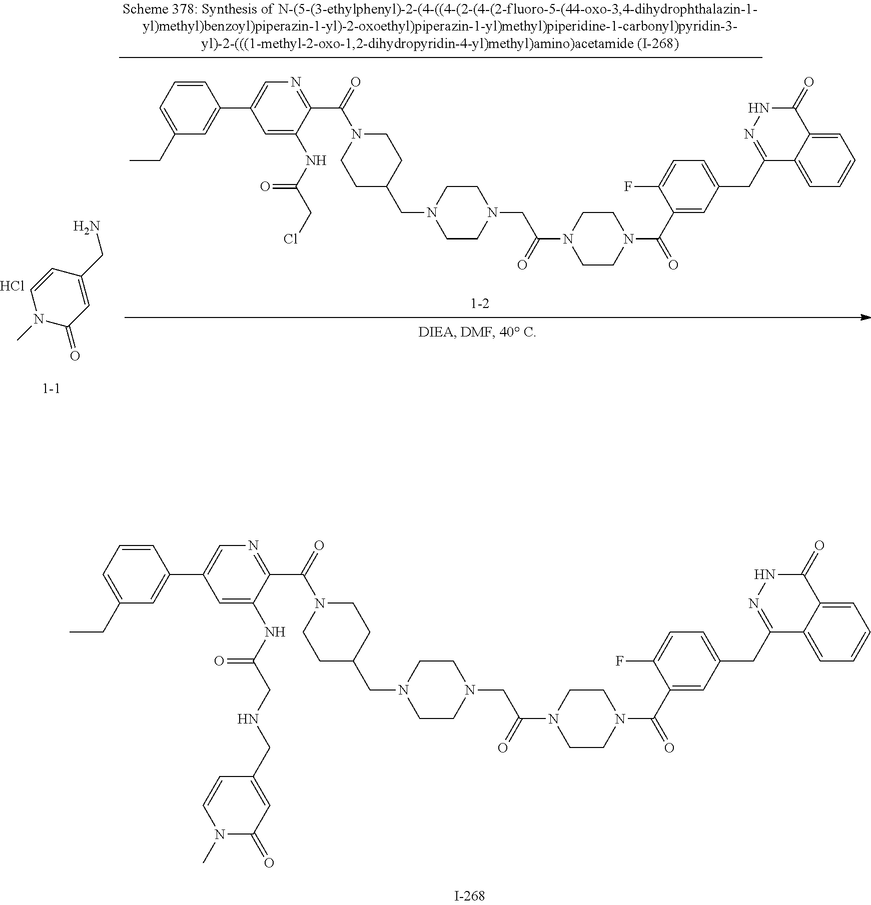

To a solution of Intermediate 1-2 (100 mg, 112.30 μmol, 1 eq) in DMF (1 mL) was added DIEA (43.54 mg, 336.91 μmol, 58.68 μL, 3 eq) and Intermediate 1-1 (23.53 mg, 134.76 μmol, 1.2 eq, HCl salt). The mixture was stirred at 40° C. for 12 h. Without workup, the crude product was purified by prep-HPLC (column: Phenomenex luna C18 150*25 mm*10 um; mobile phase: [water (FA)-ACN]; gradient: 3%-33% B over 20 min) and the eluent was lyophilized to give I-268 (30.8 mg, 30.38 μmol, 27.06% yield, 97.876% purity) as a white solid. LCMS (Method D): Retention time: 0.345 min, [M+H]⁺=992.3. ¹H NMR (400 MHz, METHANOL-d₄) δ=8.77 (d, J=1.6 Hz, 1H), 8.60 (d, J=1.6 Hz, 1H), 8.41-8.30 (m, 2H), 7.99-7.91 (m, 1H), 7.90-7.80 (m, 2H), 7.61 (d, J=7.2 Hz, 1H), 7.55-7.47 (m, 3H), 7.43 (t, J=7.6 Hz, 1H), 7.39-7.29 (m, 2H), 7.17 (t, J=9.2 Hz, 1H), 6.63 (s, 1H), 6.54 (d, J=6.8 Hz, 1H), 4.70 (d, J=12.8 Hz, 2H), 4.38 (s, 2H), 3.88-3.72 (m, 5H), 3.67 (s, 2H), 3.56-3.42 (m, 9H), 3.35 (s, 1H), 3.18 (t, J=12.4 Hz, 1H), 3.04-2.68 (m, 12H), 2.67-2.58 (m, 2H), 2.04-2.02 (m, 1H), 1.91 (d, J=12.4 Hz, 1H), 1.76 (d, J=12.8 Hz, 1H), 1.37-1.22 (m, 5H) ¹⁹F NMR (377 MHz, METHANOL-d₄) δ=−120.77.

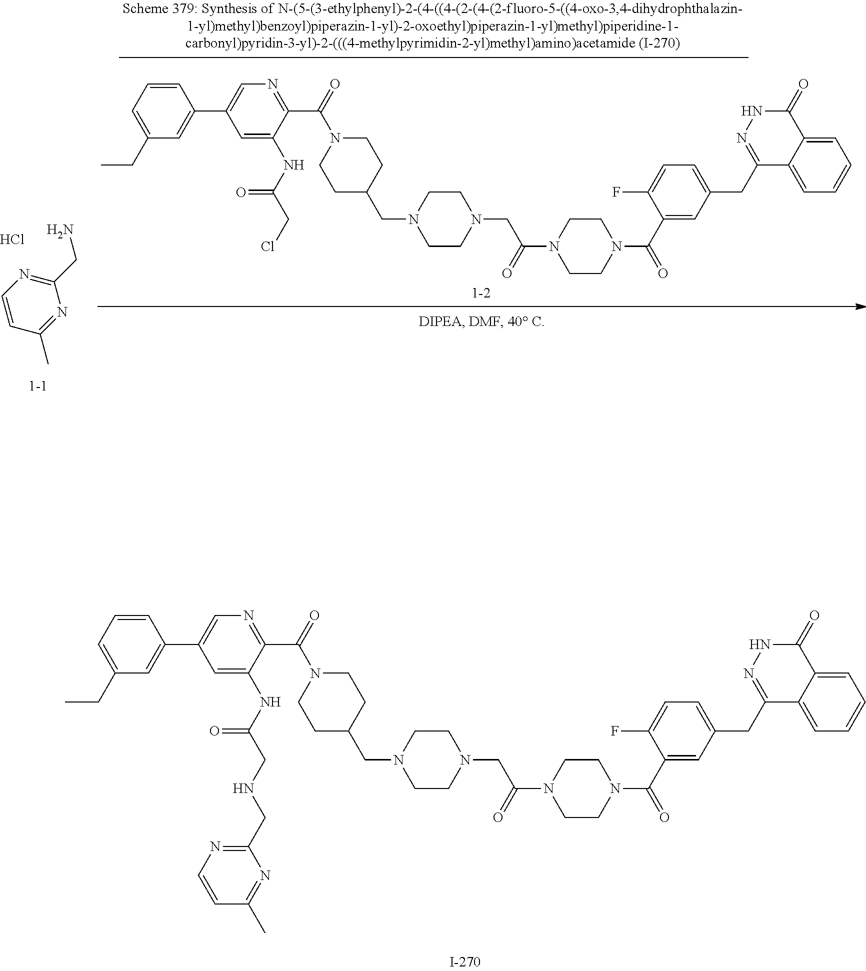

To a solution of Intermediate 1-2 (100 mg, 112.30 μmol, 1 eq) and Intermediate 1-1 (21.51 mg, 134.76 μmol, 1.2 eq, HCl salt) in DMF (1 mL) was added DIEA (43.54 mg, 336.91 gmol, 58.68 μL, 3 eq). The mixture was stirred at 60° C. for 3 h. Without workup, the crude product was purified by prep-HPLC (column: Welch Ultimate C18 150*25 mm*5 um; mobile phase: [water (TFA)-ACN]; gradient: 15%-35% B over 13 min) and the eluent was lyophilized to give I-270 (15.71 mg, 14.32 gmol, 12.75% yield, 99.486% purity, TFA salt) as a yellow solid. LCMS (Method D): Retention time: 0.322 min, [M+H]⁺=977.4. ¹H NMR (400 MHz, METHANOL-d₄) δ=8.74-8.65 (m, 2H), 8.41-8.30 (m, 2H), 7.98 (s, 1H), 7.91-7.81 (m, 2H), 7.56-7.48 (m, 3H), 7.46-7.31 (m, 4H), 7.17 (t, J=8.8 Hz, 1H), 4.67 (d, J=12.4 Hz, 1H), 4.56 (s, 2H), 4.39 (s, 2H), 4.26 (s, 2H), 3.88-3.44 (m, 10H), 3.28-3.17 (m, 5H), 3.07 (s, 4H), 3.01-2.84 (m, 4H), 2.78-2.72 (m, 2H), 2.65-2.54 (m, 3H), 2.19-2.10 (m, 1H), 2.00-1.93 (m, 1H), 1.82-1.74 (m, 1H), 1.61-1.50 (m, 1H), 1.45-1.37 (m, 1H), 1.32-1.27 (in, 3H). ¹⁹F NMR (377 MHz, METHANOL-d₄) δ=−77.181, −120.793.

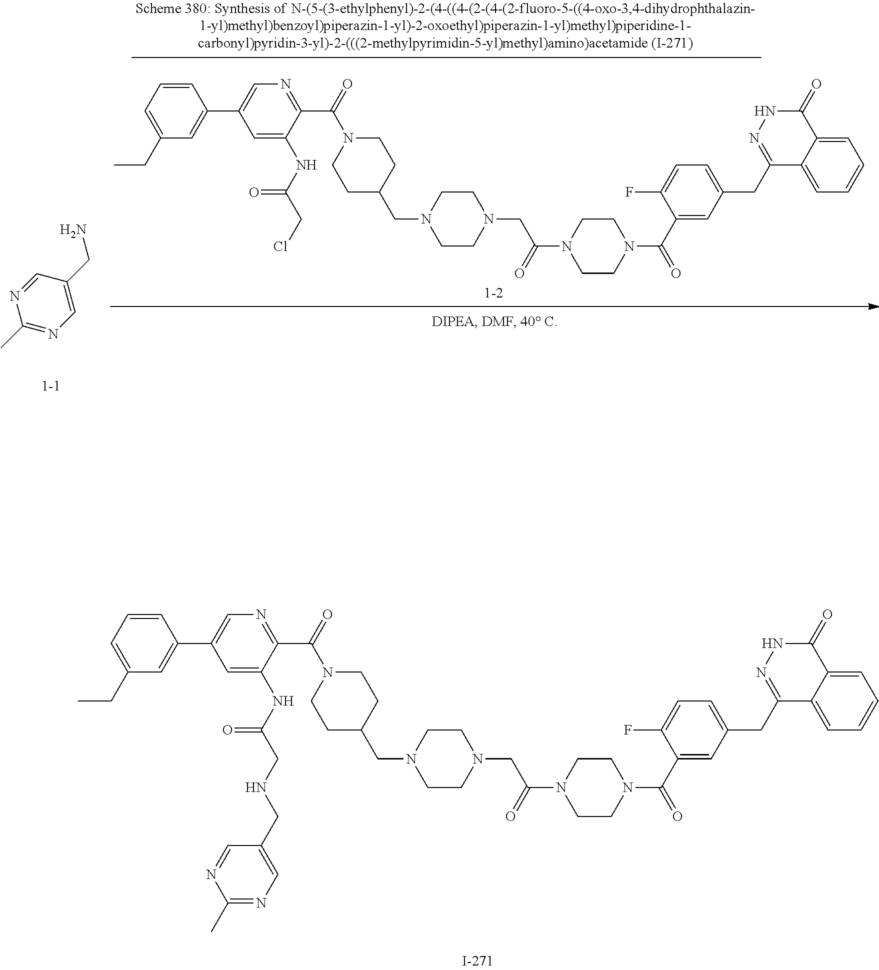

To a solution of Intermediate 1-2 (100 mg, 112.30 μmol, 1 eq) and Intermediate 1-1 (16.60 mg, 134.76 μmol, 1.2 eq) in DMF (1 mL) was added DIEA (29.03 mg, 224.61 μmol, 39.12 μL, 2 eq). The mixture was stirred at 40° C. for 1 hr. The reaction mixture was concentrated under reduced pressure to give a residue. The residue was purified by prep-HPLC (column: Phenomenex luna C18 150*25 mm*10 um; mobile phase: [water (FA)-ACN]; gradient: 5%-35% B over 8 min) and the eluent was concentrated to remove ACN and lyophilized to give product. I-271 (36 mg, 35.70 μmol, 31.79% yield, 96.905% purity) was obtained as a white solid. LCMS (Method D): Retention time: 0.321 min, [M+H]⁺=977.3. ¹H NMR (400 MHz, METHANOL-d₄) δ=8.86-8.82 (m, 2H), 8.60 (d, J=2.0 Hz, 1H), 8.37 (d, J=7.2 Hz, 2H), 7.95 (s, 1H), 7.91-7.76 (m, 2H), 7.67-7.25 (m, 6H), 7.17 (t, J=8.8 Hz, 1H), 4.72 (d, J=13.2 Hz, 1H), 4.39 (s, 2H), 3.96-3.83 (m, 3H), 3.83-3.73 (m, 2H), 3.68 (d, J=4.8 Hz, 2H), 3.55-3.44 (m, 5H), 3.38 (s, 3H), 3.21-3.12 (m, 1H), 2.99-2.60 (m, 14H), 2.51-2.46 (m, 2H), 2.06-1.84 (m, 2H), 1.82-1.70 (m, 1H), 1.42-1.15 (m, 5H). ¹⁹F NMR (377 MHz, METHANOL-d₄) δ=−120.801.

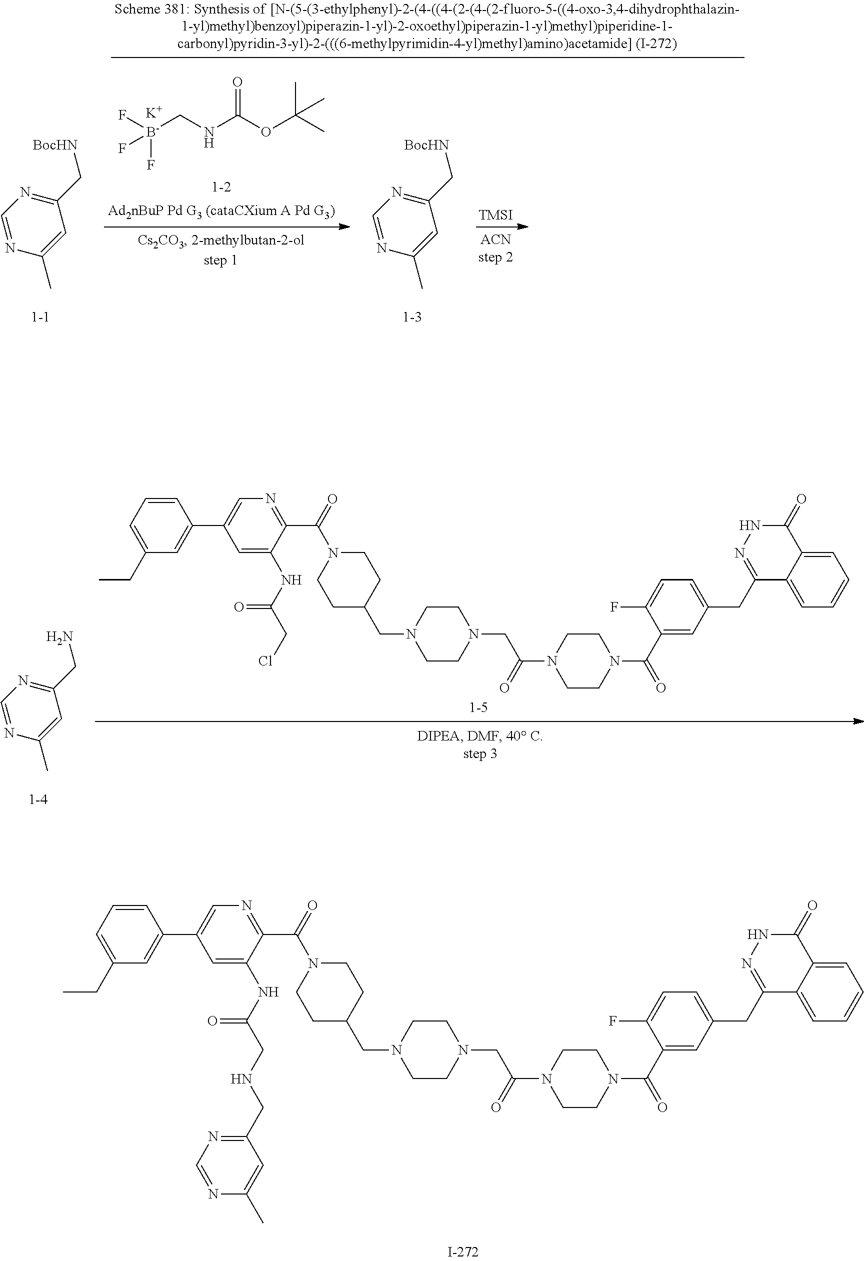

### Step 1: Synthesis of Intermediate 1-3

A mixture of Intermediate 1-1 (400 mg, 2.31 mmol, 1 eq), Intermediate 1-2 (657.72 mg, 2.77 mmol, 1.2 eq), Cs₂CO₃ (1.5 M, 4.62 mL, 3 eq), and Ad₂nBuP Pd G3(cataCXium A Pd G₃) (336.75 mg, 462.40 μmol, 0.2 eq) in 2-methylbutan-2-ol (4 mL) was degassed and purged with N₂ 3 times, and then the mixture was stirred at 90° C. for 16 hr under N₂ atmosphere. The reaction mixture was concentrated under reduced pressure to give a residue. The residue was purified by flash silica gel chromatography (ISCO; 80 g SepaFlash Silica Flash Column, Eluent of 0˜80% Ethyl acetate/Petroleum ether gradient @100 mL/min) and was concentrated under reduced pressure to give Intermediate 1-3 (250 mg, 1.12 mmol, 48.43% yield) as a yellow oil. LCMS (Method D): Retention time: 0.223 min, [M+H]⁺=224.1. ¹H NMR (400 MHz, CHLOROFORM-d) δ=8.94 (s, 1H), 7.09 (s, 1H), 4.31 (d, J=5.6 Hz, 2H), 2.46 (s, 3H), 1.40 (s, 9H).

### Step 2: Synthesis of Intermediate 1-4

To a solution of Intermediate 1-3 (50 mg, 223.94 μmol, 1 eq) in ACN (1 mL) was added TMSI (268.86 mg, 1.34 mmol, 182.90 μL, 6 eq). The mixture was stirred at 0° C. for 0.5 hr. The reaction mixture was concentrated under reduced pressure to give Intermediate 1-4 (27 mg, crude) as a yellow solid. LCMS (Method D): Retention time: 0.1 min, [M+H]⁺=124.0.

### Step 3: Synthesis of I-272

To a solution of Intermediate 1-4 (16.60 mg, 134.76 μmol, 1.2 eq) and Intermediate 1-5 (100 mg, 112.30 μmol, 1 eq) in DMF (0.5 mL) was added DIEA (29.03 mg, 224.61 μmol, 39.12 μL, 2 eq). The mixture was stirred at 40° C. for 0.5 hr. The reaction mixture was diluted with H₂O (6 mL) and extracted with EA (5 mL*2). The combined organic layers were washed with NaCl (aq) (5 mL*2), dried over Na₂SO₄, filtered and concentrated under reduced pressure to give a residue. The residue was purified by prep-HPLC (column: Phenomenex luna C18 150*25 mm*10 um; mobile phase: [water (FA)-ACN]; gradient: 6%-36% B over 10 min) and the eluent was concentrated to remove ACN and lyophilized to give I-272 (18.06 mg, 18.39 μmol, 16.38% yield, 99.518% purity) as a white solid. LCMS (Method D): Retention time: 0.322 min, [M+H]⁺=977.6. ¹H NMR (400 MHz, METHANOL-d₄) δ=8.94 (s, 1H), 8.79-8.73 (m, 1H), 8.64-8.57 (m, 1H), 8.39-8.34 (m, 1H), 7.99-7.92 (m, 1H), 7.90-7.81 (m, 2H), 7.69 (s, 1H), 7.57-7.47 (m, 3H), 7.45-7.41 (m, 1H), 7.40-7.30 (m, 2H), 7.17 (m, 1H), 4.58 (br s, 1H), 4.39 (s, 2H), 3.97 (s, 2H), 3.85-3.72 (m, 3H), 3.68 (s, 2H), 3.56-3.33 (m, 8H), 3.21-3.11 (m, 2H), 2.90-2.66 (m, 10H), 2.57 (s, 3H), 2.50-2.40 (m, 2H), 2.03-1.83 (m, 2H), 1.81-1.70 (m, 1H), 1.39-1.24 (m, 5H). ¹⁹F NMR (377 MHz, METHANOL-d₄) δ=−120.800.

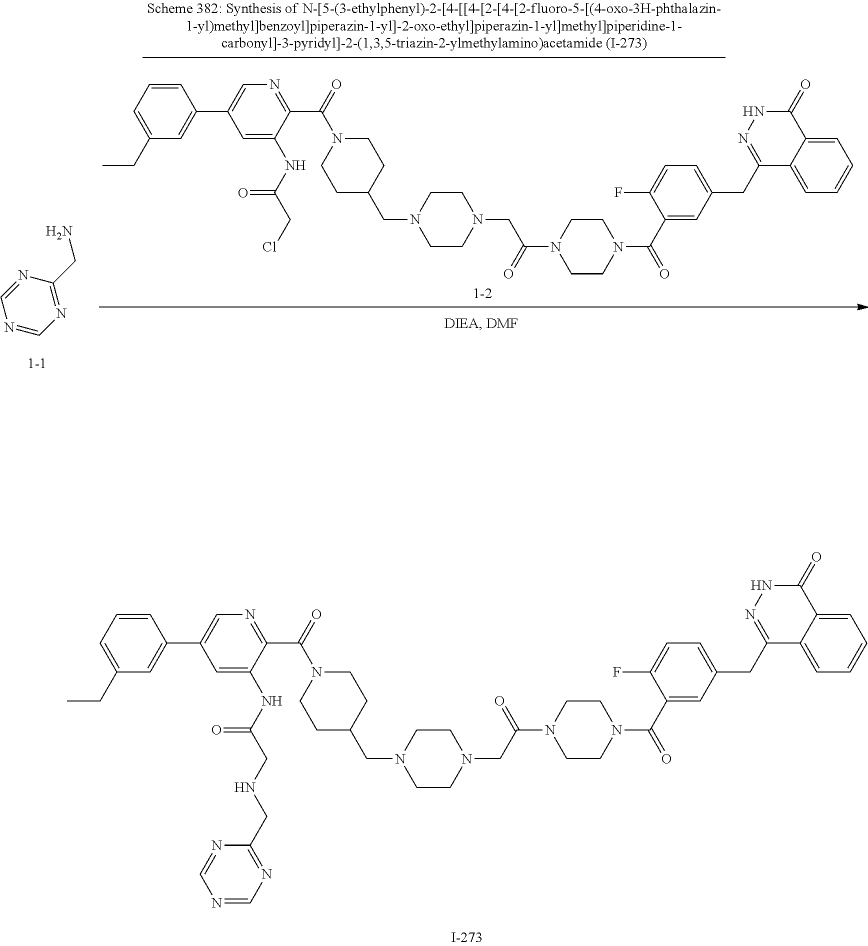 [SMILES: NCc1ncncn1 ; CCc1cccc(-c2cnc(C(=O)N3CCC(CN4CCN(CC(=O)N5CCN(C(=O)c6cc(Cc7n[nH]c(=O)c8ccccc78)ccc6F)CC5)CC4)CC3)c(NC(=O)CCl)c2)c1 ; CCc1cccc(-c2cnc(C(=O)N3CCC(CN4CCN(CC(=O)N5CCN(C(=O)c6cc(Cc7n[nH]c(=O)c8ccccc78)ccc6F)CC5)CC4)CC3)c(NC(=O)CNCc3ncncn3)c2)c1]

A solution of Intermediate 1-1 (74.20 mg, 673.82 μmol, 3 eq) and Intermediate 1-2 (200 mg, 224.61 μmol, 1 eq) in DMF (2 mL) was added DIEA (87.09 mg, 673.82 μmol, 117.37 μL, 3 eq). The reaction mixture was stirred and refluxed at 60° C. for 0.5 h. The reaction was diluted with water (2 mL) and extracted with DCM (2 mL*3), the combined organic phase was dried by Na₂SO₄, and concentrated under reduced pressure to give a residue. The crude was purified by prep-HPLC (column: Welch Ultimate C18 150*25 mm*5 um; mobile phase: [water (TFA)-ACN]; gradient: 12%-42% B over 10 min) the eluent was concentrated to remove ACN and lyophilized to give product. I-273 (7 mg, 6.18 μmol, 2.75% yield, 95.111% purity, TFA salt) was obtained as a brown solid. LCMS (Method D): Retention time: 0.326 min, [M+H]⁺=964.4. ¹H NMR (400 MHz, METHANOL-d₄) δ=8.71-8.63 (m, 1H), 8.37 (d, J=7.2 Hz, 1H), 8.25 (s, 1H), 7.99-7.93 (m, 1H), 7.90-7.81 (m, 2H), 7.56-7.47 (m, 4H), 7.46-7.41 (m, 1H), 7.41-7.30 (m, 3H), 7.17 (t, J=8.8 Hz, 1H), 4.67-4.60 (m, 1H), 4.39 (s, 2H), 3.87-3.58 (m, 8H), 3.58-3.41 (m, 3H), 3.30-3.10 (m, 6H), 3.09-2.98 (m, 4H), 2.98-2.82 (m, 4H), 2.80-2.79 (m, 1H), 2.81-2.68 (m, 2H), 2.19-2.06 (m, 1H), 1.99-1.89 (m, 1H), 1.80-1.69 (m, 1H), 1.63-1.47 (m, 1H), 1.29 (t, J=7.6 Hz, 5H). ¹⁹F NMR (377 MHz, METHANOL-d₄) δ=−77.084, −120.792.

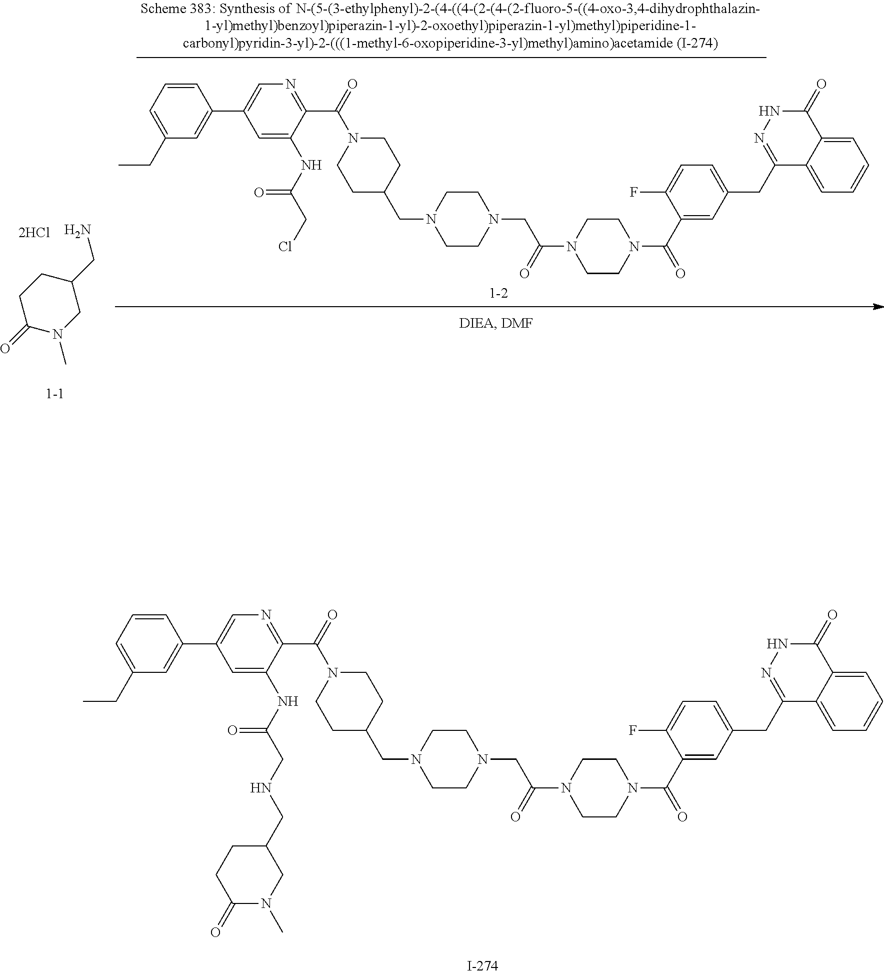

A solution of Intermediate 1-1 (44.14 mg, 247.07 μmol, 1.1 eq, HCl salt) and Intermediate 1-2 (200 mg, 224.61 μmol, 1 eq) in DMF (2 mL) was added DIEA (87.09 mg, 673.82 μmol, 117.37 μL, 3 eq). The reaction mixture was stirred and refluxed at 40° C. for 2 h. The reaction was diluted with water (2 mL) and extracted with DCM (2 mL*3), the combined organic phase was dried by Na₂SO₄, and concentrated under reduced pressure to give a residue. The crude was purified by prep-HPLC (column: Phenomenex luna C18 150*25 mm*10 um; mobile phase: [water (FA)-ACN]; gradient: 8%-38% B over 10 min) and the eluent was concentrated to remove ACN and lyophilized to give product. I-274 (50 mg, 47.29 mol, 20.20% yield, 94.587% purity, FA salt) was obtained as a white solid. LCMS (Method D): Retention time: 0.326 min, [M+H]⁺=996.5. SFC: Retention time: 3.906 min, 4.314 min. ¹H NMR (400 MHz, METHANOL-d₄) δ=8.82 (s, 1H), 8.60 (s, 1H), 8.43 (s, 1H), 8.37 (d, J=7.6 Hz, 1H), 7.99-7.92 (m, 1H), 7.91-7.79 (m, 2H), 7.56-7.47 (m, 3H), 7.43 (t, J=7.6 Hz, 1H), 7.39-7.33 (m, 1H), 7.31 (d, J=7.6 Hz, 1H), 7.16 (t, J=8.8 Hz, 1H), 4.68 (d, J=12.0 Hz, 1H), 4.38 (s, 2H), 3.88-3.77 (m, 2H), 3.76-3.63 (m, 3H), 3.59-3.49 (m, 3H), 3.49-3.42 (m, 2H), 3.40 (s, 1H), 3.33 (s, 3H), 3.25-3.12 (m, 2H), 3.01-2.88 (m, 4H), 2.79-2.79 (m, 1H), 2.81-2.62 (m, 10H), 2.48-2.35 (m, 4H), 2.18-2.07 (m, 1H), 2.05-1.91 (m, 3H), 1.82-1.71 (m, 1H), 1.63-1.51 (m, 1H), 1.29 (t, J=7.6 Hz, 6H). ¹⁹F NMR (377 MHz, METHANOL-d₄) δ=−120.787.

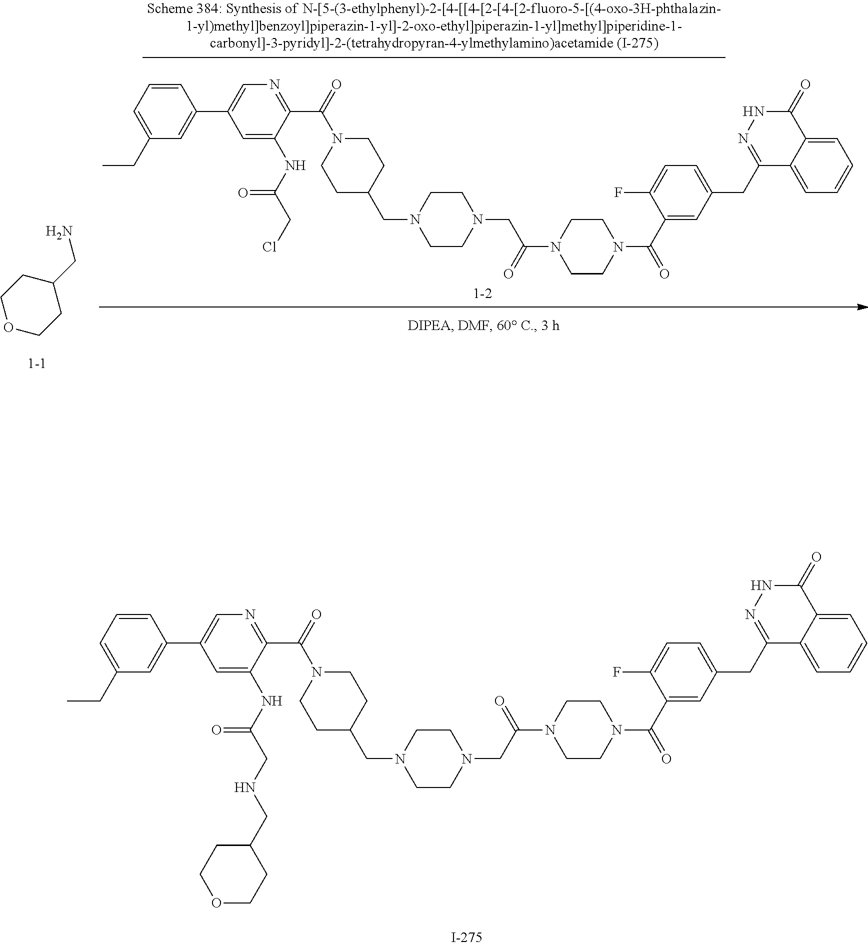 [SMILES: NCC1CCOCC1 ; CCc1cccc(-c2cnc(C(=O)N3CCC(CN4CCN(CC(=O)N5CCN(C(=O)c6cc(Cc7n[nH]c(=O)c8ccccc78)ccc6F)CC5)CC4)CC3)c(NC(=O)CCl)c2)c1 ; CCc1cccc(-c2cnc(C(=O)N3CCC(CN4CCN(CC(=O)N5CCN(C(=O)c6cc(Cc7n[nH]c(=O)c8ccccc78)ccc6F)CC5)CC4)CC3)c(NC(=O)CNCC3CCOCC3)c2)c1]

To a mixture of Intermediate 1-1 (100 mg, 110.06 μmol, 1.2 eq) and Intermediate 1-2 (10.56 mg, 91.71 μmol, 1.00 eq) in DMF (1 mL) was added DIEA (23.71 mg, 183.43 μmol, 31.95 μL, 2 eq). The mixture was stirred at 60° C. for 3 hr. The reaction was poured into H₂O (1 mL) and extracted with EA (3 mL*3). The combined organic layers were dried over Na₂SO₄, filtered and concentrated under reduced pressure to give the crude product. The residue was purified by prep-HPLC (column: Waters xbridge 150*25 mm 10 um; mobile phase: [water (NH₄HCO₃)-ACN]; gradient: 38%-68% B over 11 min) followed by lyophilization. I-275 (38 mg, 38.60 μmol, 42.09% yield, 98.457% purity) was obtained as a white solid. LCMS (Method H): Rt=0.637 min, [M+H]⁺=969.1. ¹H NMR (400 MHz, METHANOL-d₄) δ=8.84 (s, 1H), 8.59 (s, 1H), 8.37 (d, J=7.6 Hz, 1H), 7.98-7.92 (m, 1H), 7.90-7.81 (m, 2H), 7.54-7.47 (m, 3H), 7.43 (t, J=7.6 Hz, 1H), 7.40-7.35 (m, 1H), 7.31 (d, J=7.6 Hz, 1H), 7.16 (t, J=9.2 Hz, 1H), 4.71-4.65 (m, 1H), 4.57 (s, 2H), 4.39 (s, 2H), 4.00-3.93 (m, 2H), 3.76 (d, J=19.2 Hz, 4H), 3.67 (d, J=1.6 Hz, 1H), 3.59-3.47 (m, 3H), 3.47-3.41 (m, 2H), 3.40 (d, J=3.6 Hz, 2H), 3.34 (d, J=6.4 Hz, 1H), 3.27 (s, 1H), 3.21-3.11 (m, 2H), 2.97-2.87 (m, 1H), 2.78-2.72 (m, 2H), 2.57-2.46 (m, 8H), 2.29-2.21 (m, 2H), 1.99-1.87 (m, 2H), 1.83-1.72 (m, 4H), 1.38-1.33 (m, 1H), 1.29 (t, J=7.6 Hz, 5H), 1.26-1.17 (m, 1H). ¹⁹F NMR (377 MHz, METHANOL-d₄) δ=−120.727.

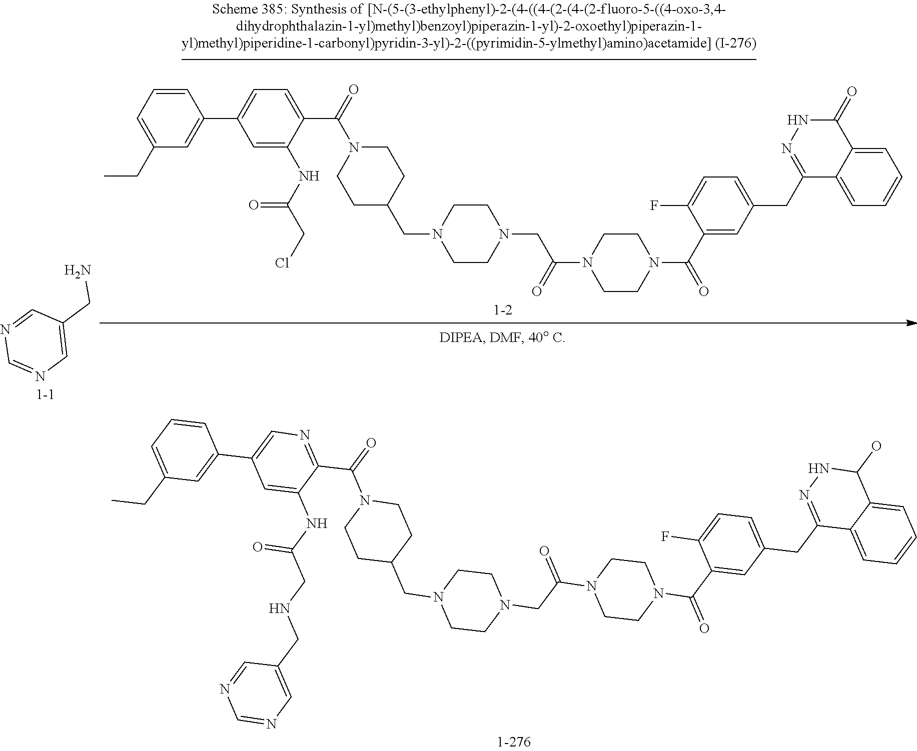 [SMILES: NCc1cncnc1 ; CCc1cccc(-c2ccc(C(=O)N3CCC(CN4CCN(CC(=O)N5CCN(C(=O)c6cc(Cc7n[nH]c(=O)c8ccccc78)ccc6F)CC5)CC4)CC3)c(NC(=O)CCl)c2)c1 ; CCc1cccc(-c2cnc(C(=O)N3CCC(CN4CCN(CC(=O)N5CCN(C(=O)c6cc(CC7=NNC(O)c8ccccc87)ccc6F)CC5)CC4)CC3)c(NC(=O)CNCc3cncnc3)c2)c1]

To a solution of Intermediate 1-1 (14.71 mg, 134.76 μmol, 1.2 eq) and Intermediate 1-2 (100 mg, 112.30 μmol, 1 eq) in DMF (1 mL) was added DIEA (29.03 mg, 224.61 μmol, 39.12 μL, 2 eq). The mixture was stirred at 40° C. for 1 hr. The reaction mixture was diluted with H₂O (6 mL) and extracted with EA (5 mL*2). The combined organic layers were washed with NaCl (aq) (5 mL*2), dried over Na₂SO₄, filtered and concentrated under reduced pressure to give a residue. The residue was purified by prep-HPLC (column: Phenomenex luna C18 150*25 mm*10 um; mobile phase: [water (FA)-ACN]; gradient: 6%-36% B over 10 min) the eluent was concentrated to remove ACN and lyophilized to give I-276 (32.51 mg, 33.22 μmol, 29.58% yield, 98.427% purity) as a white solid. LCMS (Method D): Retention time: 0.351 min, [M+H]⁺=963.5. ¹H NMR (400 MHz, METHANOL-d₄) δ=9.08 (s, 1H), 8.97 (s, 2H), 8.86 (s, 1H), 8.60 (s, 1H), 8.49-8.40 (m, 1H), 8.37 (d, J=7.6 Hz, 1H), 7.99-7.93 (m, 1H), 7.90-7.80 (m, 2H), 7.56-7.46 (m, 3H), 7.45-7.41 (m, 1H), 7.40-7.34 (m, 1H), 7.32 (d, J=7.2 Hz, 1H), 7.19-7.15 (m, 1H), 4.73-4.68 (m, 1H), 4.57 (s, 2H), 4.39 (s, 2H), 3.94-3.83 (m, 3H), 3.82-3.63 (m, 5H), 3.58-3.45 (m, 5H), 3.40 (s, 1H), 3.19-3.13 (m, 1H), 2.94-2.87 (m, 1H), 2.84-2.51 (m, 10H), 2.45-2.40 (m, 1H), 2.02-1.87 (m, 2H), 1.81-1.72 (m, 1H), 1.43 (d, J=8.0 Hz, 1H), 1.31-1.28 (m, 4H). ¹⁹F NMR (377 MHz, METHANOL-d₄) δ=−120.802.

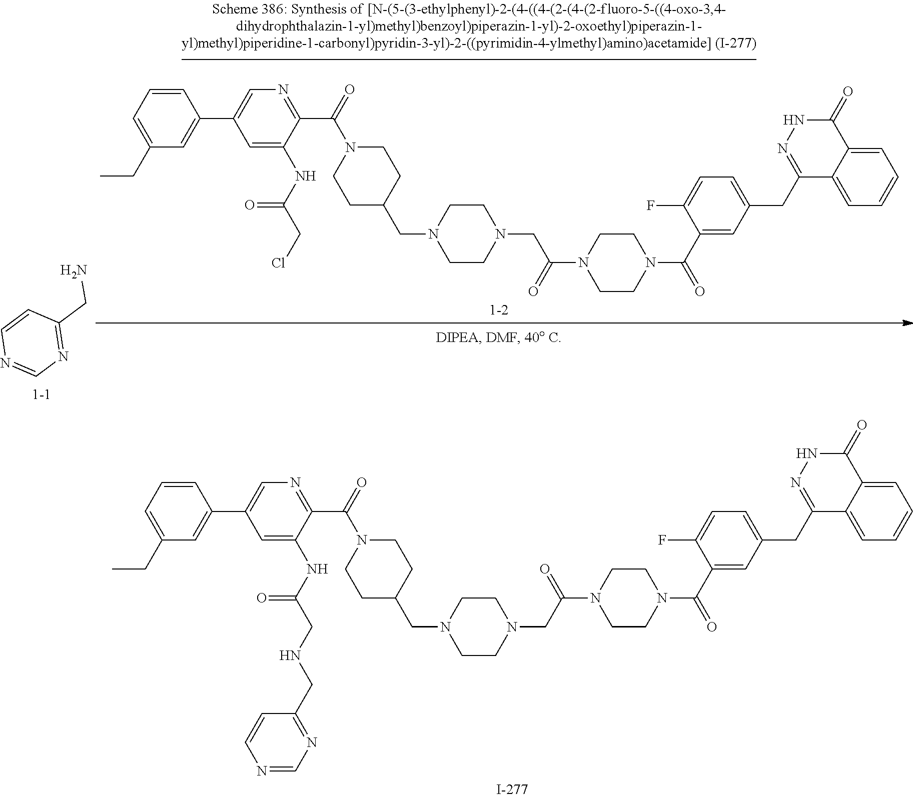 [SMILES: NCc1ccncn1 ; CCc1cccc(-c2cnc(C(=O)N3CCC(CN4CCN(CC(=O)N5CCN(C(=O)c6cc(Cc7n[nH]c(=O)c8ccccc78)ccc6F)CC5)CC4)CC3)c(NC(=O)CCl)c2)c1 ; CCc1cccc(-c2cnc(C(=O)N3CCC(CN4CCN(CC(=O)N5CCN(C(=O)c6cc(Cc7n[nH]c(=O)c8ccccc78)ccc6F)CC5)CC4)CC3)c(NC(=O)CNCc3ccncn3)c2)c1]

To a solution of Intermediate 1-2 (100 mg, 112.30 μmol, 1 eq) and Intermediate 1-1 (14.71 mg, 134.76 μmol, 1.2 eq) in DMF (1 mL) was added DIEA (29.03 mg, 224.61 μmol, 39.12 μL, 2 eq). The mixture was stirred at 40° C. for 1 hr. The reaction mixture was diluted with H₂O (6 mL) and extracted with EA (5 mL*2). The combined organic layers were washed with NaCl (aq) (5 mL*2), dried over Na₂SO₄, filtered and concentrated under reduced pressure to give a residue. The residue was purified by prep-HPLC (column: Phenomenex luna C18 150*25 mm*10 um; mobile phase: [water (FA)-ACN]; gradient: 6%-36% B over 10 min) the eluent was concentrated to remove ACN and lyophilized to give I-277 (21.6 mg, 21.57 μmol, 19.21% yield, 96.192% purity) as a pink solid. LCMS (Method D): Retention time: 0.351 min, [M+H]⁺=963.4. ¹H NMR (400 MHz, METHANOL-d₄) δ=9.10 (s, 1H), 8.79-8.74 (m, 2H), 8.61 (s, 1H), 8.40-8.34 (m, 2H), 7.99-7.92 (m, 1H), 7.91-7.81 (m, 2H), 7.78 (d, J=5.2 Hz, 1H), 7.56-7.46 (m, 3H), 7.45-7.41 (m, 1H), 7.40-7.28 (m, 2H), 7.19-7.14 (m, 1H), 4.60 (d, J=12.4 Hz, 2H), 4.39 (s, 2H), 4.02 (s, 2H), 3.86-3.72 (m, 3H), 3.68 (s, 2H), 3.53 (s, 4H), 3.45 (s, 1H), 3.37 (s, 2H), 3.22-3.10 (m, 1H), 2.93-2.81 (m, 2H), 2.80-2.65 (m, 9H), 2.54-2.43 (m, 2H), 2.03-1.85 (m, 2H), 1.80-1.71 (m, 1H), 1.40-1.20 (m, 5H). ¹⁹F NMR (377 MHz, METHANOL-d₄) δ=−120.794.

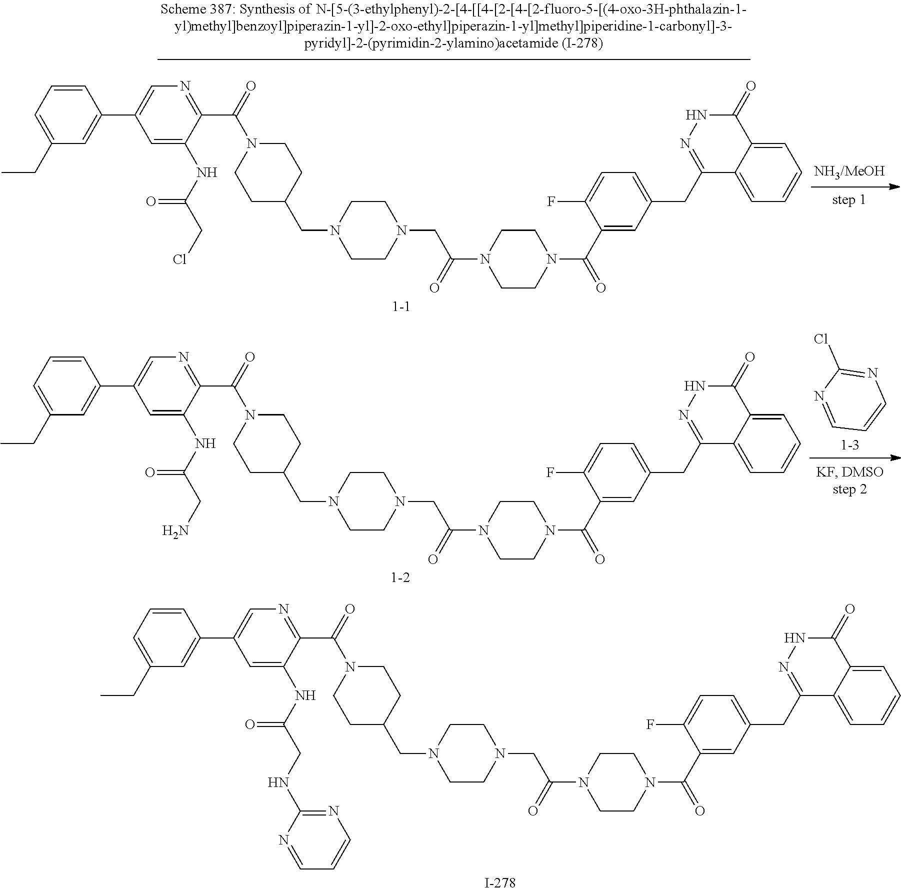

### Step 1: Synthesis of Intermediate 1-2

A solution of Intermediate 1-1 (0.5 g, 561.52 μmol, 1 eq) in NH₃/MeOH (7 M, 5 mL, 62.33 eq) was stirred and refluxed at 60° C. for 12 h. The reaction mixture was filtered to give the filtrate. The filtrate was concentrated and the residue was purified by reversed-phase HPLC (0.1% of FA) and the eluent was lyophilized to give product. Intermediate 1-2 (55 mg, 60.14 μmol, 10.71% yield, 95.24% purity) was obtained as a yellow solid. LCMS (Method D): Retention time: 0.413 min, [M+H]+=870.3.

### Step 2: Synthesis of I-278

To a solution of Intermediate 1-2 (53 mg, 60.85 μmol, 1 eq) and Intermediate 1-3 (10.45 mg, 106.55 μmol, 1.75 eq) in DMSO (0.5 mL) was added KF (10.61 mg, 182.55 μmol, 3 eq) at 25° C., then the mixture was stirred at 60° C. for 1 hr. The reaction mixture was filtered to give the filtrate. The residue was purified by reversed-phase HPLC (0.1% of FA) and the eluent was lyophilized to give product. I-278 (7 mg, 6.96 μmol, 11.44% yield, 94.419% purity) was obtained as a white solid. LCMS (Method D): Retention time: 0.366 min, [M+H]+=949.4. ¹H NMR (400 MHz, METHANOL-d₄) δ=8.67 (s, 1H), 8.58 (d, J=2.0 Hz, 1H), 8.40-8.33 (m, 3H), 7.99-7.92 (m, 1H), 7.91-7.81 (m, 2H), 7.54-7.47 (m, 3H), 7.45-7.41 (m, 1H), 7.41-7.35 (m, 1H), 7.31 (d, J=7.2 Hz, 1H), 7.17 (t, J=8.8 Hz, 1H), 6.76 (t, J=4.8 Hz, 1H), 4.58 (s, 2H), 4.51-4.44 (m, 1H), 4.39 (s, 2H), 4.16 (s, 2H), 3.81-3.66 (m, 5H), 3.57-3.49 (m, 2H), 3.28-3.20 (m, 2H), 3.10-3.00 (m, 1H), 2.78-2.70 (m, 3H), 2.62-2.40 (m, 8H), 2.27-2.18 (m, 2H), 1.89-1.79 (m, 2H), 1.75-1.66 (m, 1H), 1.29 (t, J=7.6 Hz, 5H). ¹⁹F NMR (377 MHz, METHANOL-d₄) δ=−120.807.

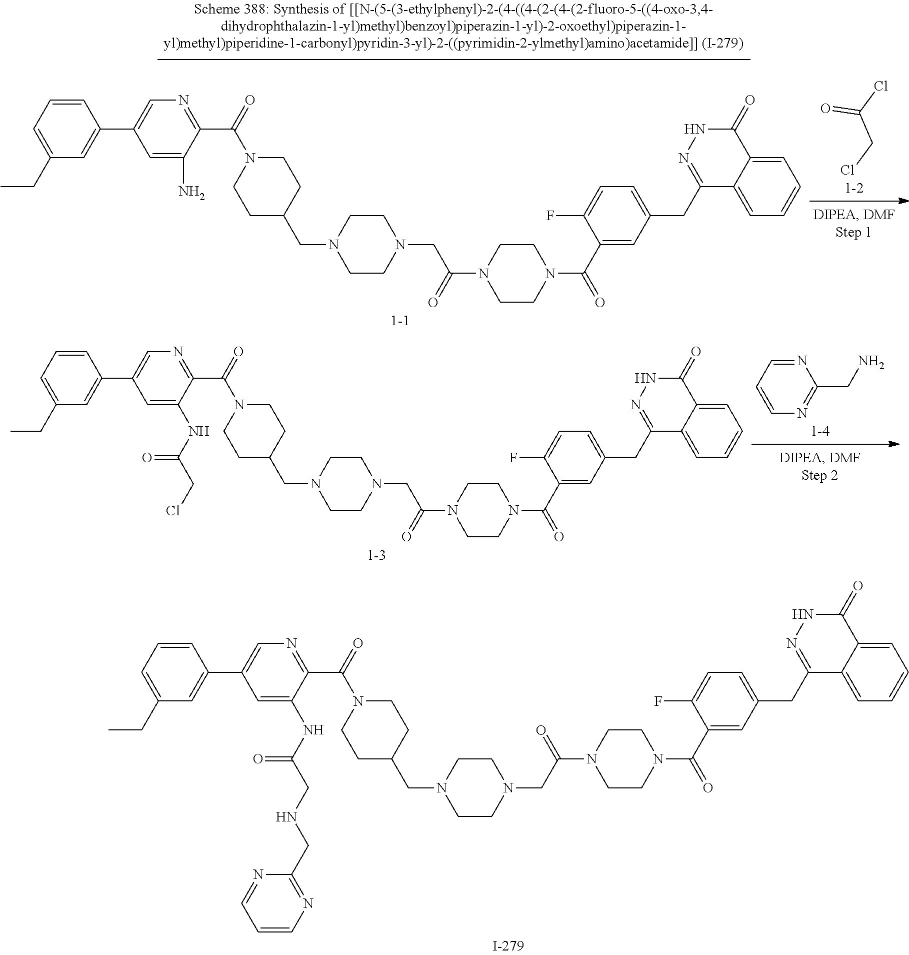

### Step 1: Synthesis of Intermediate 1-3

To a mixture of Intermediate 1-1 (10.69 g, 12.57 mmol, 1 eq, HCl salt) and Intermediate 1-2 (1.70 g, 15.08 mmol, 1.20 mL, 1.2 eq) in DMF (100 mL) was added DIEA (3.25 g, 25.14 mmol, 4.38 mL, 2 eq). The mixture was stirred at 25° C. for 1 hr. The reaction mixture was concentrated under reduced pressure to give a residue. The residue was purified by flash silica gel chromatography (ISCO; 120 g SepaFlash Silica Flash Column, Eluent of 0˜50% Ethyl acetate/Methanol gradient @100 mL/min) and then the eluent was concentrated under reduced pressure to give a residue. The residue was further purified by prep-HPLC (column: Phenomenex luna C18 (250*70 mm, 10 um); mobile phase: [water(FA)-ACN]; gradient: 15%-45% B over 22 min) followed by lyophilization to give Intermediate 1-3 (2.4 g, 2.56 mmol, 20.37% yield, 95% purity) as a yellow oil. LCMS (Method D): Rt=0.374 min, [M+H]⁺=890.6. ¹H NMR (400 MHz, METHANOL-d4) δ=8.66 (d, J=1.6 Hz, 1H), 8.45 (d, J=1.6 Hz, 1H), 8.36 (d, J=7.6 Hz, 1H), 7.98-7.92 (m, 1H), 7.91-7.80 (m, 2H), 7.55-7.47 (m, 3H), 7.43 (t, J=7.6 Hz, 1H), 7.40-7.34 (m, 1H), 7.32 (d, J=7.2 Hz, 1H), 7.16 (t, J=9.2 Hz, 1H), 4.66 (d, J=12.4 Hz, 1H), 4.38 (s, 2H), 4.29 (s, 2H), 3.90-3.63 (m, 8H), 3.61-3.49 (m, 4H), 3.26-3.16 (m, 4H), 3.03 (d, J=3.6 Hz, 1H), 2.96 (s, 1H), 2.92 (d, J=11.6 Hz, 3H), 2.79-2.69 (m, 4H), 2.09 (s, 1H), 1.96 (d, J=16.4 Hz, 1H), 1.78 (d, J=12.4 Hz, 1H), 1.56-1.40 (m, 2H), 1.29 (t, J=7.6 Hz, 3H). ¹⁹F NMR (377 MHz, METHANOL-d₄) δ=−120.770.

### Step 2: Synthesis of I-279

To a solution of Intermediate 1-3 (100 mg, 112.30 μmol, 1 eq) in DMF (1 mL) was added DIPEA (43.54 mg, 336.91 μmol, 58.68 μL, 3 eq) and Intermediate 1-4 (12.26 mg, 112.30 μmol, 1 eq). The mixture was stirred at 40° C. for 16 hr. The reaction mixture was filtered and then the filtrate was purified by prep-HPLC (column: Phenomenex luna C18 150*25 mm*10 um; mobile phase: [water (FA)-ACN]; gradient: 4%-34% B over 10 min). The eluent was concentrated under reduced pressure to remove ACN and lyophilized to give I-279 (15.50 mg, 15.76 μmol, 14.04% yield, 97.94% purity) as a yellow solid. LCMS (Method D): Rt=0.358 min, [M+H]⁺=963.3. ¹H NMR (400 MHz, METHANOL-d₄) δ=8.84-8.80 (m, 1H), 8.66 (d, J=17.2 Hz, 2H), 8.51-8.34 (m, 2H), 8.01-7.81 (m, 3H), 7.61-7.49 (m, 3H), 7.47-7.31 (m, 4H), 7.23-7.13 (m, 1H), 4.69-4.57 (m, 1H), 4.40 (s, 2H), 4.17-4.15 (m, 1H), 3.84-3.73 (m, 3H), 3.69 (s, 2H), 3.65-3.49 (m, 4H), 3.49-3.44 (m, 1H), 3.38 (d, J=12.0 Hz, 3H), 3.23-3.12 (m, 1H), 3.01-2.44 (m, 14H), 2.07-1.96 (m, 1H), 1.95-1.71 (m, 2H), 1.43-1.36 (m, 1H), 1.36-1.25 (m, 4H). ¹⁹F NMR (376 MHz, METHANOL-d₄) δ=−120.755.

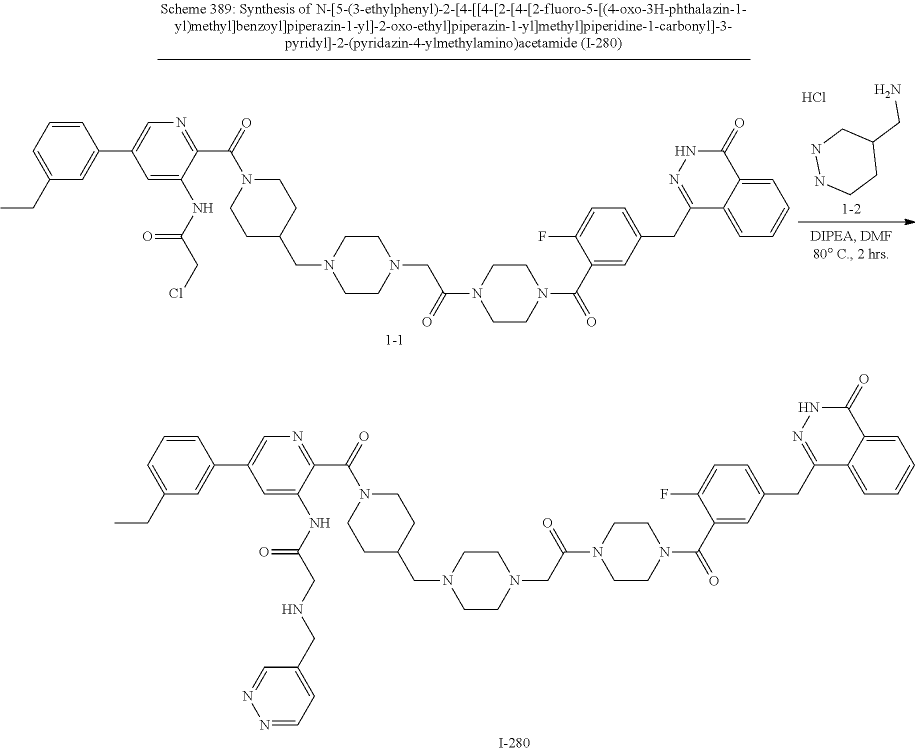

To a solution of Intermediate 1-1 (100 mg, 112.30 μmol, 1 eq) in DMF (1 mL) was added Intermediate 1-2 (24.53 mg, 168.46 μmol, 1.5 eq) and DIPEA (43.54 mg, 336.91 μmol, 58.68 μL, 3 eq). The mixture was stirred at 80° C. for 2 hrs. The mixture was filtered. The filtrate was purified by reversed-phase HPLC (0.1% FA condition). The eluent was concentrated under reduced pressure to remove ACN and then lyophilized to give I-280 (27.28 mg, 27.53 μmol, 24.52% yield, 97.2% purity, FA salt) as a pink solid. LCMS (Method D): Rt: 0.346 min, [M+H]⁺=963.5. ¹H NMR (400 MHz, METHANOL-d₄) δ=9.38 (s, 1H), 9.15 (d, J=5.2 Hz, 1H), 8.86 (s, 1H), 8.59 (s, 1H), 8.52-8.44 (m, 1H), 8.36 (d, J=7.6 Hz, 1H), 7.98-7.92 (m, 1H), 7.92-7.80 (m, 3H), 7.53 (s, 1H), 7.52-7.46 (m, 2H), 7.45-7.40 (m, 1H), 7.40-7.34 (m, 1H), 7.31 (d, J=7.6 Hz, 1H), 7.19-7.12 (m, 1H), 4.67 (d, J=13.2 Hz, 1H), 4.38 (s, 2H), 3.97 (s, 2H), 3.90-3.83 (m, 1H), 3.82-3.77 (m, 1H), 3.76-3.72 (m, 1H), 3.72-3.64 (m, 2H), 3.56-3.52 (m, 1H), 3.50 (s, 3H), 3.34 (s, 2H), 3.27 (s, 1H), 3.20-3.07 (m, 1H), 2.93-2.82 (m, 1H), 2.78-2.51 (m, 10H), 2.38-2.25 (m, 2H), 1.97-1.83 (m, 2H), 1.81-1.70 (m, 1H), 1.34-1.25 (m, 4H), 1.22-1.08 (m, 1H). ¹⁹F NMR (376 MHz, METHANOL-d₄) δ=−120.787.

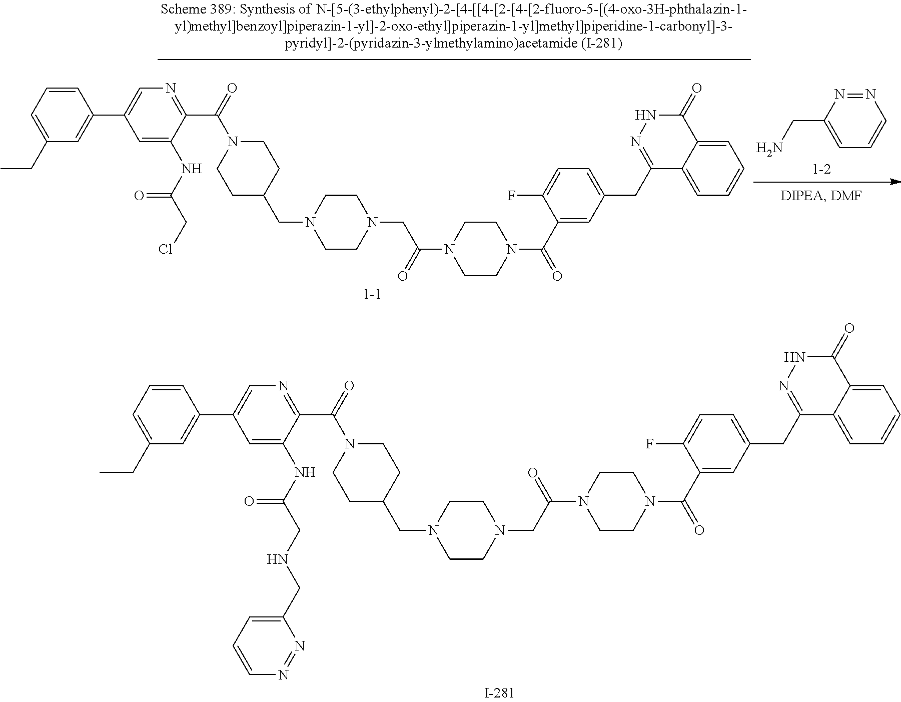

To a mixture of Intermediate 1-1 (100 mg, 112.30 μmol, 1 eq) and Intermediate 1-2 (24.51 mg, 224.61 μmol, 2 eq) in DMF (1 mL) was added DIEA (43.54 mg, 336.91 μmol, 58.68 μL, 3 eq), then the mixture was stirred at 40° C. for 1 hour. The reaction mixture was diluted with water (30 mL) and extracted with EA (5 mL*2). The combined organic layers were concentrated under reduced pressure to give a residue. The crude product was purified by prep-HPLC (column: Phenomenex luna C18 150*25 mm*10 um; mobile phase: [water (FA)-ACN]; gradient: 8%-38% B over 10 min) to give I-281 (27.94 mg, 26.47 μmol, 23.57% yield, 95.6% purity, FA salt) as a white solid. L CMS (Method D): Retention time: 0.329 min, [M+H]⁺=963.4. ¹H NMR (400 MHz, METHANOL-d₄) δ=9.11 (d, J=4.8 Hz, 1H), 8.74 (s, 1H), 8.61 (s, 1H), 8.37 (d, J=7.6 Hz, 1H), 8.34 (s, 1H), 8.03 (d, J=8.4 Hz, 1H), 7.99-7.92 (m, 1H), 7.91-7.81 (m, 2H), 7.76-7.54 (m, 1H), 7.57-7.47 (m, 3H), 7.43 (t, J=7.6 Hz, 1H), 7.40-7.28 (m, 2H), 7.17 (t, J=9.2 Hz, 1H), 4.60 (d, J=13.2 Hz, 1H), 4.39 (s, 2H), 4.19 (s, 2H), 3.86-3.72 (m, 3H), 3.67 (s, 2H), 3.57-3.46 (m, 5H), 3.41 (s, 3H), 3.17 (t, J=12.4 Hz, 1H), 2.99-2.70 (m, 11H), 2.64-2.51 (m, 2H), 2.07-1.95 (m, 1H), 1.88 (d, J=11.6 Hz, 1H), 1.76 (d, J=13.2 Hz, 1H), 1.41-1.19 (in, 5H). ¹⁹F NMR (376 MHz, METHANOL-d₄) δ=−120.79.

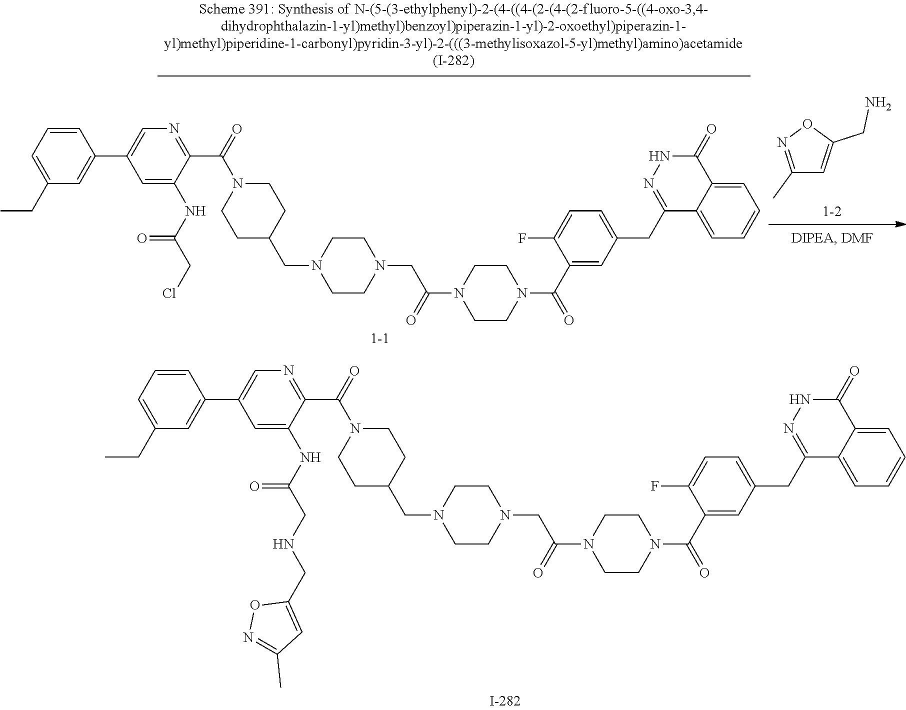

To a solution of Intermediate 1-2 (25.19 mg, 224.61 μmol, 2 eq) and Intermediate 1-1 (100 mg, 112.30 μmol, 1 eq) in DMF (1 mL) was added DIEA (43.54 mg, 336.91 μmol, 58.68 μL, 3 eq). The mixture was stirred at 60° C. for 2 hr. The reaction mixture was diluted with water (6 mL) and extracted with ethyl acetate (2 mL×3). The combined organic layers were washed with brine (2 mL×3) and dried over sodium sulfate, filtered and concentrated under reduced pressure to give a residue. The residue was purified by prep-HPLC (column: Phenomenex luna C18 150*25 mm*10 um; mobile phase: [water (FA)-ACN]; gradient: 12%-42% B over 10 min). The eluent was concentrated under reduced pressure to remove ACN and then lyophilized to give I-282 (27.83 mg, 27.74 μmol, 24.70% yield, 96.294% purity) as a white solid. LCMS (Method D): Rt: 0.344 min, [M+H]⁺=966.4. ¹H NMR (400 MHz, METHANOL-d₄) δ=8.74 (s, 1H), 8.60 (s, 1H), 8.37 (d, J=7.6 Hz, 1H), 8.00-7.91 (m, 1H), 7.90-7.79 (m, 2H), 7.56-7.47 (m, 3H), 7.43-7.41 (m, 1H), 7.39-7.33 (m, 1H), 7.31 (d, J=7.6 Hz, 1H), 7.18-7.15 (m, 1H), 6.33 (s, 1H), 4.66 (d, J=12.4 Hz, 1H), 4.38 (s, 2H), 3.96 (s, 2H), 3.84-3.73 (m, 3H), 3.67 (s, 2H), 3.52 (s, 2H), 3.46 (s, 3H), 3.39-3.33 (m, 2H), 3.19-3.17 (m, 1H), 2.98-2.62 (m, 12H), 2.53-2.50 (m, 2H), 2.26 (s, 3H), 2.05-1.86 (m, 2H), 1.76 (d, J=12.0 Hz, 1H), 1.35 (d, J=13.6 Hz, 1H), 1.31-1.27 (m, J=7.6 Hz, 4H). ¹⁹F NMR (377 MHz, METHAN OL-d₄) δ=−120.779.

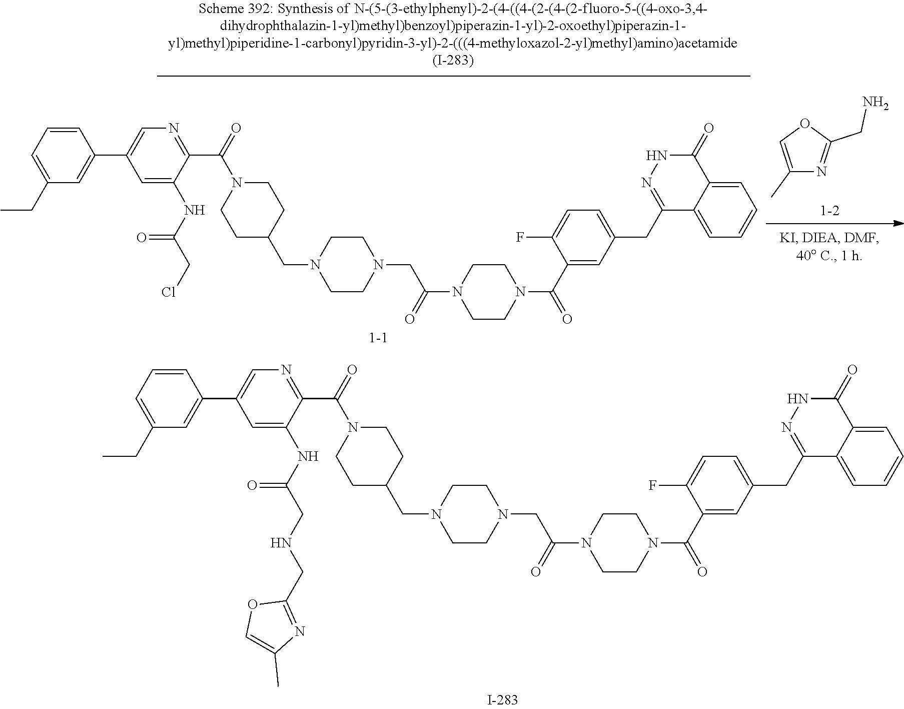

To a solution of Intermediate 1-1 (80 mg, 89.84 μmol, 1 eq) in DMF (1.5 mL) was added DIEA (46.45 mg, 359.37 μmol, 62.60 μL, 4 eq), KI (59.66 mg, 359.37 μmol, 4 eq) and Intermediate 1-2 (30.48 mg, 134.76 μmol, 1.5 eq, TFA salt). The mixture was stirred at 40° C. for 1 h. The mixture was filtered. The filtrate was purified by Prep-HPLC (column: Waters Xbridge 150*25 mm*5 um; mobile phase: [water (NH₄HCO₃)-ACN]; gradient: 28%-58% B over 15 min) to afford I-283 (8.22 mg, 7.35 μmol, 8.18% yield, 96.572% purity, TFA salt) as a white solid. LCMS (Method G): Rt=0.612 min, [M+H]⁺=966.6. ¹H NMR (400 MHz, METHANOL-d₄) δ=8.70 (s, 1H), 8.60 (d, J=2.0 Hz, 1H), 8.40-8.33 (m, 1H), 7.98-7.92 (m, 1H), 7.90-7.81 (m, 2H), 7.58-7.45 (m, 4H), 7.43-7.29 (m, 3H), 7.16 (m, 1H), 4.67-4.61 (m, 1H), 4.38 (s, 2H), 3.94 (s, 2H), 3.86-3.58 (m, 6H), 3.57-3.46 (m, 4H), 3.27-3.02 (m, 3H), 2.93-2.85 (m, 1H), 2.78-2.72 (m, 2H), 2.65-2.35 (m, 8H), 2.35-2.25 (m, 2H), 2.12 (s, 3H), 1.96-1.86 (m, 2H), 1.79-1.71 (m, 1H), 1.33-1.25 (m, 6H).

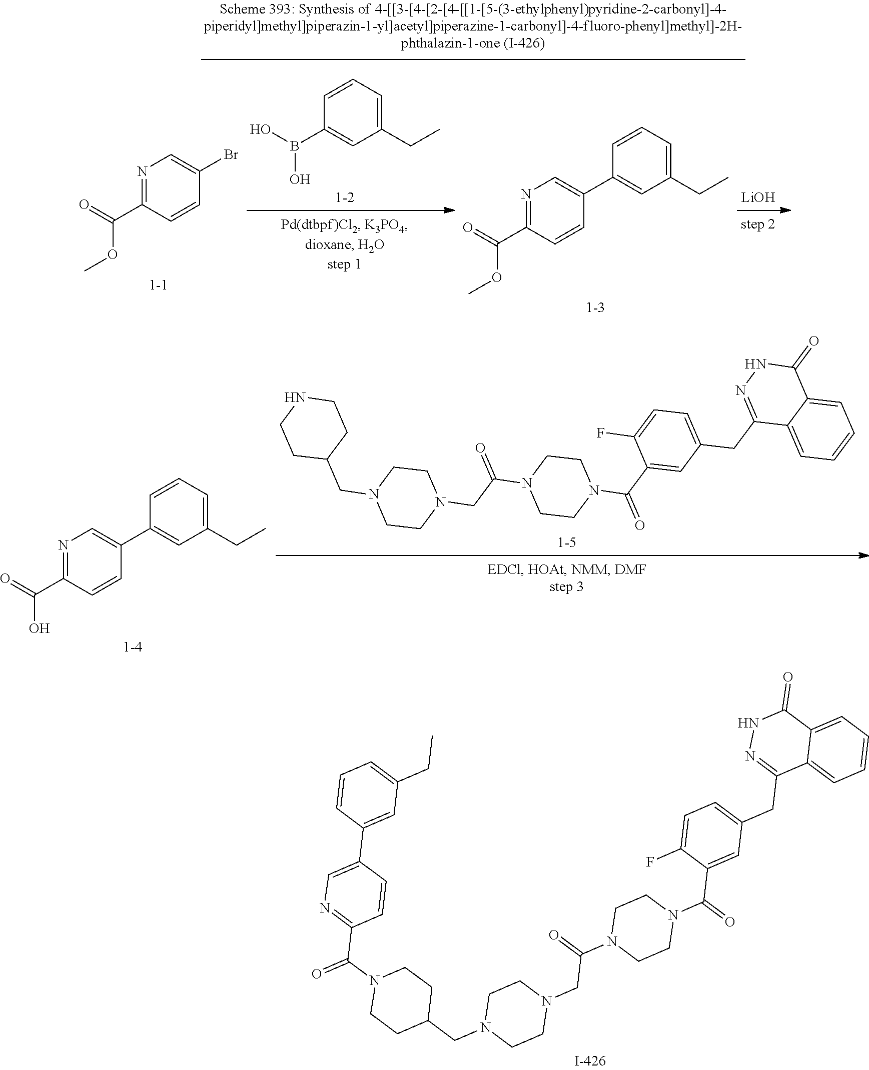

### Step 1: Synthesis of Intermediate 1-3

To a solution of intermediate 1-1 (200 mg, 925.79 gmol, 1 eq) and Intermediate 1-2 (208.28 mg, 1.39 mmol, 1.5 eq) in H₂O (0.4 mL) and dioxane (2 mL) was added K₃PO₄ (589.54 mg, 2.78 mmol, 3 eq) and ditert-butyl(cyclopentyl)phosphane;dichloropalladium;iron (60.34 mg, 92.58 μmol, 0.1 eq), then the mixture was stirred at 80° C. for 1 hour under N₂. The reaction mixture was diluted with water (80 mL) and extracted with EA (30 mL*2). The combined organic layers were concentrated under reduced pressure to give a residue. The residue was purified by flash silica gel chromatography (ISCO; 20 g SepaFlash Silica Flash Column, Eluent of 0˜10% Dichloromethane: Methanol@60 mL/min). The eluent was concentrated under reduced pressure to give the product. Intermediate 1-3 (150 mg, 621.67 μmol, 67.15% yield) was obtained as a white solid. LCMS (Method D): Rt: 0.478 min, (M+H)=241.9.

### Step 2: Synthesis of Intermediate 1-4

To a solution of intermediate 1-3 (120 mg, 497.34 μmol, 1 eq) in THF (2 mL), MeOH (1 mL) and H₂O (0.5 mL) was added LiOH·H₂O (62.61 mg, 1.49 mmol, 3 eq), then the mixture was stirred at 20° C. for 0.5 hour. The reaction mixture was concentrated under reduced pressure to remove THF and MeOH. 1 M HCl (about 1 mL) was added to adjust the to pH=3˜4, and the mixture was extracted with EA (10 mL*3). The combined organic layers were concentrated under reduced pressure to give a residue. Intermediate 1-4 (100 mg, 440.03 μmol, 88.48% yield) was obtained as a white solid.

### Step 3: Synthesis of I-426

To a solution of intermediate 1-4 (50 mg, 220.01 μmol, 1 eq) and intermediate 1-5 (129.74 mg, 220.01 μmol, 1 eq) in DMF (2 mL) was added HOAt (29.95 mg, 220.01 μmol, 30.78 μL, 1 eq), NMM (111.27 mg, 1.10 mmol, 120.94 μL, 5 eq) and EDCI (126.53 mg, 660.04 μmol, 3 eq), then the mixture was stirred at 20° C. for 1 hour. The reaction mixture was concentrated under reduced pressure to give a residue. The crude product was purified by reversed-phase HPLC (0.1% FA condition). The eluent was concentrated under reduced pressure to remove ACN and then lyophilized to give the product. I-426 (32.68 mg, 36.99 μmol, 16.81% yield, 95.644% purity, FA salt) was obtained as a yellow solid. LCMS (Method D): Rt: 0.371 min, (M+H)=799.3. ¹H NMR (400 MHz, METHANOL-d4) δ=8.83 (s, 1H), 8.49 (s, 1H), 8.37 (d, J=7.6 Hz, 1H), 8.19-8.17 (m, 1H), 7.99-7.91 (m, 1H), 7.91-7.81 (m, 2H), 7.64 (d, J=8.0 Hz, 1H), 7.57-7.47 (m, 3H), 7.45-7.35 (m, 2H), 7.31 (d, J=7.6 Hz, 1H), 7.16 (t, J=8.9 Hz, 1H), 4.66 (d, J=12.4 Hz, 1H), 4.39 (s, 2H), 3.89-3.61 (m, 6H), 3.56-3.49 (m, 2H), 3.34 (s, 3H), 3.15 (t, J=12.8 Hz, 1H), 2.92 (t, J=12.4 Hz, 1H), 2.78-2.53 (m, 10H), 2.37-2.32 (m, 2H), 1.98-1.89 (m, 2H), 1.81-1.72 (m, 1H), 1.31-1.27 (m, 5H). ¹⁹F NMR (400 MHz, METHANOL-d4) δ=−120.800.

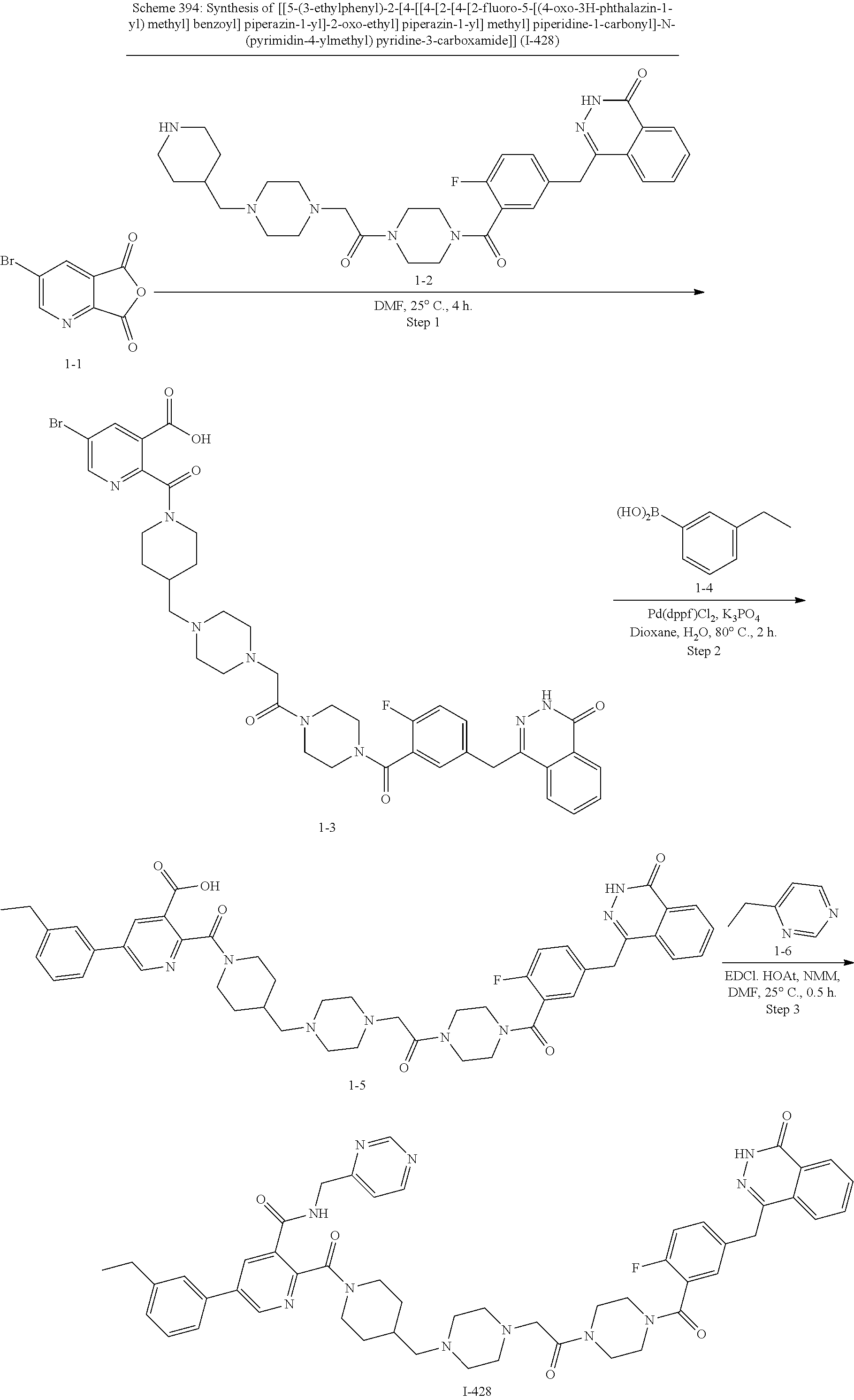

### Step 1: Synthesis of Intermediate 1-3

To a solution of intermediate 1-1 (728.24 mg, 3.19 mmol, 2 eq) in DMF (10 mL) was added intermediate 1-2 (1 g, 1.60 mmol, 1 eq, HCl salt). The mixture was stirred at 25° C. for 4 hr. To the mixture was added H₂O (5 mL) to give a residue. The residue was purified by reversed-phase chromatography (neutral condition) and the eluent was concentrated to remove MeCN and then lyophilized. Intermediate 1-1 (589 mg, crude) was obtained as a yellow solid. LCMS (Method D): Rt=0.278 min, (M+H)=817.2.

### Step 2: Synthesis of Intermediate 1-5

A mixture of intermediate 1-3 (589 mg, 720.31 μmol, 1 eq), intermediate 1-4 (140.44 mg, 936.41 μmol, 1.3 eq), ditert-butyl(cyclopentyl)phosphane;dichloropalladium;iron (46.95 mg, 72.03 μmol, 0.1 eq) and K₃PO₄ (458.69 mg, 2.16 mmol, 3 eq) in dioxane (6 mL) and H₂O (1.2 mL) was degassed and purged with N₂ 3 times, and then the mixture was stirred at 80° C. for 2 hr under N₂ atmosphere. The mixture was concentrated under reduced pressure to give a residue. The residue was purified by reversed-phase chromatography (neutral condition) and the eluent was concentrated to remove MeCN and then lyophilized. Intermediate 1-5 (380 mg, crude) was obtained as a brown solid. LCMS (Method D): Rt=0.359 min, (M+H)=843.3.

### Step 3: Synthesis of I-428

To a solution of intermediate 1-5 (50 mg, 59.32 μmol, 1 eq) and intermediate 1-6 (6.47 mg, 59.32 μmol, 1 eq) in DMF (0.5 mL) was added EDCI (34.11 mg, 177.95 μmol, 3 eq), HOAt (8.07 mg, 59.32 μmol, 8.30 μL, 1 eq) and NMM (30.00 mg, 296.58 μmol, 32.61 μL, 5 eq). The mixture was stirred at 25° C. for 0.5 hr. The reaction mixture was poured into H₂O (2 mL) and extract with EA (2 mL*3). The combined organic layers were dried over Na₂SO₄, filtered and concentrated under reduced pressure to give a residue. The residue was purified by prep-HPLC (column: Phenomenex luna C18 150*25 mm*10 um; mobile phase: [water (FA)-ACN]; gradient: 12%-42% B over 8 min) and concentrated under reduced pressure to remove ACN, and lyophilized to give product. I-428 (12.22 mg, 12.98 μmol, 21.88% yield, 99.223% purity) was obtained as a yellow solid. LCMS (Method D): Rt=0.342 min, (M+H)=934.5. ¹H NMR (400 MHz, METHANOL-d4) δ=9.10 (s, 1H), 8.94 (d, J=2.0 Hz, 1H), 8.75 (d, J=5.6 Hz, 1H), 8.49 (d, J=2.0 Hz, 1H), 8.37 (dd, J=1.2, 7.6 Hz, 1H), 8.00-7.92 (m, 1H), 7.91-7.80 (m, 2H), 7.66-7.62 (m, 2H), 7.60 (d, J=8.0 Hz, 1H), 7.52-7.44 (m, 2H), 7.40-7.32 (m, 2H), 7.17 (t, J=9.2 Hz, 1H), 4.75-4.55 (m, 4H), 4.39 (s, 2H), 3.78 (d, J=19.6 Hz, 2H), 3.67 (s, 2H), 3.54-3.46 (m, 4H), 3.41 (s, 2H), 3.20-3.11 (m, 1H), 3.03-2.83 (m, 5H), 2.82-2.67 (m, 6H), 2.65-2.55 (m, 2H), 2.11-1.94 (m, 1H), 1.93-1.84 (m, 1H), 1.68 (br d, J=12.6 Hz, 1H), 1.52-1.35 (m, 2H), 1.31 (t, J=7.6 Hz, 3H).

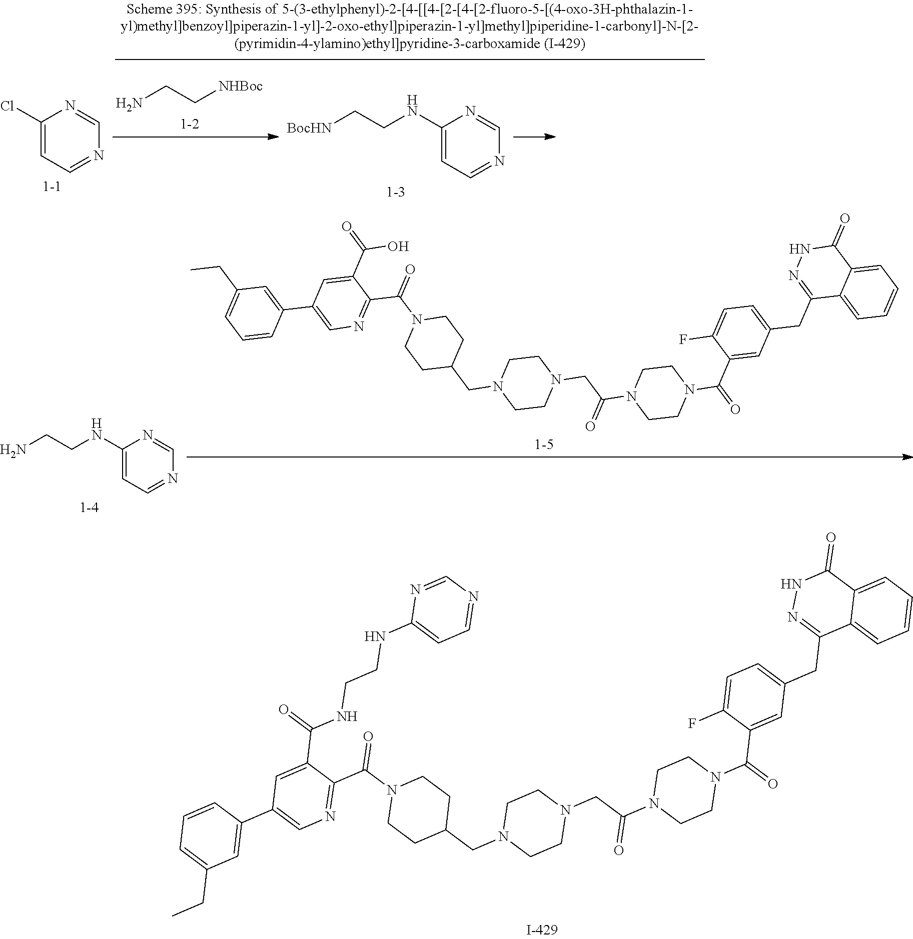

Step 1: Synthesis of Intermediate 1-3 To a solution of intermediate 1-1 (500 mg, 4.37 mmol, 1 eq) in ACN (5 mL) was added DIEA (1.13 g, 8.73 mmol, 1.52 mL, 2 eq) and Intermediate 1-2 (1.05 g, 6.55 mmol, 1.03 mL, 1.5 eq). The mixture was stirred at 25° C. for 1 hr. The residue was diluted with H₂O (5 mL) and extracted with EA (5 mL*3), dried over Na₂SO₄, filtered and concentrated under reduced pressure to give a residue. The residue was purified by flash silica gel chromatography (ISCO; 12 g SepaFlash Silica Flash Column, Eluent of 0˜100% Petroleum ether/Ethyl acetate@60 mL/min). The eluent was concentrated to afford intermediate 1-3 (300 mg, 1.26 mmol, 28.84% yield) as yellow oil. ¹H NMR (400 MHz, CHLOROFORM-d) δ=8.56 (s, 1H), 8.13 (d, J=5.2 Hz, 1H), 6.41 (d, J=5.6 Hz, 1H), 5.87 (s, 1H), 5.84-5.82 (m, 1H), 5.01 (s, 1H), 3.52 (d, J=3.2 Hz, 2H), 3.39 (d, J=4.8 Hz, 2H), 1.44 (s, 9H).

### Step 2: Synthesis of Intermediate 1-4

To a solution of intermediate 1-3 (46 mg, 193.05 μmol, 1 eq) in DCM (0.2 mL) was added HCl/dioxane (2 M, 0.4 mL, 4.14 eq). The mixture was stirred at 25° C. for 1 hr. The reaction liquid was concentrated by reduced pressure to obtain the crude product. The crude product was used in the next step without further purification. Intermediate 1-4 (33 mg, 188.97 μmol, 97.89% yield, HCl salt) was obtained as a white solid. ¹H NMR (400 MHz, METHANOL-d4) δ=8.78 (s, 1H), 8.18-8.15 (m, 1H), 6.92 (d, J=7.2 Hz, 1H), 3.94-3.90 (m, 2H), 3.25 (t, J=6.0 Hz, 2H).

### Step 3: Synthesis of I-429

To a solution of intermediate 1-5 (50 mg, 59.32 μmol, 1 eq) in DMF (0.5 mL) was added HOAt (8.07 mg, 59.32 μmol, 8.30 μL, 1 eq), EDCI (34.11 mg, 177.95 μmol, 3 eq), NMM (30.00 mg, 296.58 μmol, 32.61 L, 5 eq) and intermediate 1-4 (12.43 mg, 71.18 μmol, 1.2 eq, HCl salt). The mixture was stirred at 25° C. for 1 hr. The reaction liquid was concentrated by reducing pressure to obtain the crude product. The residue was purified by prep-HPLC (column: Phenomenex luna C18 150*25 mm*10 um; mobile phase: [water (FA)-ACN]; gradient: 5%-35% B over 8 min), and the eluent was concentrated and lyophilized to afford I-429 (10.94 mg, 11.15 μmol, 18.79% yield, 98.132% purity) as a yellow solid. LCMS (Method D): Rt=0.361 min, (M+H)=963.5. ¹H NMR (400 MHz, METHANOL-d4) δ=8.89 (d, J=2.0 Hz, 1H), 8.41-8.36 (m, 2H), 8.27 (d, J=1.6 Hz, 1H), 8.01-7.91 (m, 2H), 7.91-7.80 (m, 2H), 7.58 (s, 1H), 7.55-7.47 (m, 2H), 7.47-7.42 (m, 1H), 7.41-7.31 (m, 2H), 7.20-7.14 (m, 1H), 6.56 (d, J=3.2 Hz, 1H), 4.63 (d, J=14.0 Hz, 2H), 4.39 (s, 2H), 3.83-3.73 (m, 2H), 3.68-3.64 (m, 4H), 3.60 (d, J=4.0 Hz, 2H), 3.56-3.46 (m, 3H), 3.43 (d, J=6.8 Hz, 2H), 3.35 (s, 2H), 3.14 (t, J=13.2 Hz, 1H), 2.96-2.87 (m, 1H), 2.81-2.72 (m, 6H), 2.71-2.63 (m, 3H), 2.55-2.42 (m, 2H), 2.01-1.87 (m, 2H), 1.68 (d, J=13.6 Hz, 1H), 1.46-1.34 (m, 2H), 1.30 (t, J=7.2 Hz, 3H).

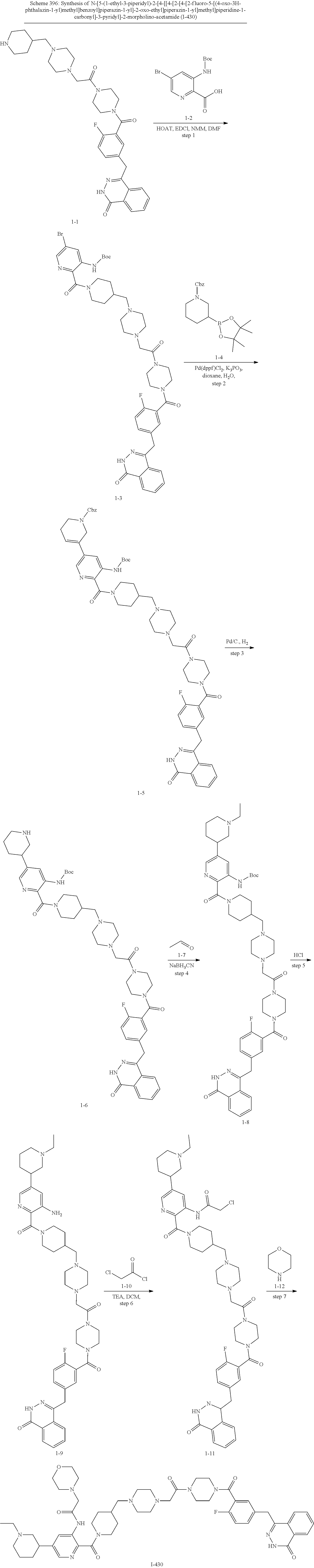

### Step 1: Synthesis of Intermediate 1-3

To a solution of intermediate 1-1 (500 mg, 847.89 μmol, 1 eq) and intermediate 1-2 (295.78 mg, 932.67 μmol, 1.1 eq) in DMF (7 mL) was added EDCI (487.62 mg, 2.54 mmol, 3 eq), HOAt (115.41 mg, 847.89 μmol, 118.61 μL, 1 eq) and NMM (428.81 mg, 4.24 mmol, 466.09 μL, 5 eq), then the mixture was stirred at 40° C. for 1 hour. The reaction mixture was diluted with water (80 mL) and extracted with DCM (30 mL*2). The combined organic layers were concentrated under reduced pressure to give a residue. The residue was purified by flash silica gel chromatography (ISCO; 20 g SepaFlash Silica Flash Column, Eluent of 0˜15% EA: Methanol@60 mL/min). The eluent was concentrated under reduced pressure to give the product. Intermediate 1-3 (500 mg, 506.29 μmol, 59.71% yield, 90% purity) was obtained as a brown solid. LCMS (Method D): Retention time: 0.366 min, (M+H)=888.3.

### Step 2: Synthesis of Intermediate 1-5

A mixture of intermediate 1-3 (500 mg, 562.54 μmol, 1 eq) and intermediate 1-4 (482.70 mg, 1.41 mmol, 2.5 eq) in dioxane (5 mL) and H₂O (1 mL) was added K₃PO₄ (358.23 mg, 1.69 mmol, 3 eq) and ditert-butyl(cyclopentyl)phosphane;dichloropalladium;iron (36.66 mg, 56.25 μmol, 0.1 eq), then the mixture was stirred at 80° C. for 1 hour under N₂. The reaction mixture was diluted with water (80 mL) and extracted with EA (30 mL*2). The combined organic layers were concentrated under reduced pressure to give a residue. The residue was purified by flash silica gel chromatography (ISCO; 20 g SepaFlash Silica Flash Column, Eluent of 0˜11% Ethyl acetate:Methanol@60 mL/min). The eluent was concentrated under reduced pressure to give the product. Intermediate 1-5 (475 mg, 438.78 μmol, 78.00% yield, 94.7% purity) was obtained as a red solid. LCMS (Method D): Retention time: 0.416 min, (M+H)=1025.3.

### Step 3: Synthesis of Intermediate 1-6

To a solution of Pd(OH)₂ (164.38 mg, 234.11 μmol, 20% purity) in MeOH (5 mL) was added intermediate 1-5 (475 mg, 463.34 μmol, 1 eq), and the mixture was degassed and purged with H₂ 3 times, and then the mixture was stirred at 40° C. for 2 hours under H₂ (15 psi) atmosphere. The reaction mixture was filtered and concentrated under reduced pressure to give a residue. Intermediate 1-6 (270 mg, 302.33 μmol, 65.25% yield) was obtained as a brown solid. LCMS (Method D): Retention time: 0.290 min, (M+H)=893.5.

### Step 4: Synthesis of Intermediate 1-8

To a solution of intermediate 1-6 (250 mg, 279.94 μmol, 1 eq) and intermediate 1-7 (21.14 mg, 335.92 μmol, 26.93 μL, 1.2 eq) in DCM (3 mL) was added AcOH (33.62 mg, 559.87 μmol, 32.05 μL, 2 eq) and NaBH(OAc)₃ (296.65 mg, 1.40 mmol, 5 eq), then the mixture was stirred at 20° C. for 5 min. To the reaction mixture was added a NaHCO₃ solution (about 10 mL) to adjust the pH to 7-8, and the mixture was extracted with EA (10 mL*3). The combined organic layers were concentrated under reduced pressure to give a residue. The crude product was purified by reversed-phase HPLC (0.1% NH₃·H₂O condition). The eluent was concentrated under reduced pressure to remove ACN and then lyophilized to give the product. Intermediate 1-8 (70 mg, 71.06 μmol, 25.38% yield, 93.5% purity) was obtained as a white solid. LCMS (Method F): Retention time: 0.709 min, (M+H)=921.5.

### Step 5: Synthesis of Intermediate 1-9

To a solution of intermediate 1-8 (60 mg, 65.14 μmol, 1 eq) in DCM (1 mL) was added HCl/dioxane (2 M, 162.85 μL, 5 eq). The mixture was stirred at 20° C. for 1 hour. The reaction mixture was concentrated under reduced pressure to give a residue. Intermediate 1-9 (50 mg, 58.31 μmol, 89.52% yield, HCl salt) was obtained as a white solid. LCMS (Method D): Retention time: 0.483 min, (M+H)=821.6.

### Step 6: Synthesis of Intermediate 1-11

To a solution of intermediate 1-9 (50 mg, 58.31 μmol, 1 eq, HCl salt) in DCM (1 mL) was added TEA (17.70 mg, 174.94 μmol, 24.35 μL, 3 eq) and intermediate 1-10 (6.59 mg, 58.31 μmol, 4.64 μL, 1 eq) at 0° C., then the mixture was stirred at 0° C. for 1 hour. The reaction mixture was diluted with water (60 mL) and extracted with DCM (15 mL*2). The combined organic layers were concentrated under reduced pressure to give a residue. Intermediate 1-11 (45 mg, 50.14 μmol, 85.99% yield) was obtained as a red solid. LCMS (Method D): Retention time: 0.262 min, (M+H)=897.4.

### Step 7: Synthesis of I-430

A solution of intermediate 1-11 (45 mg, 50.14 μmol, 1 eq) and Intermediate 1-12 (21.84 mg, 250.70 μmol, 22.06 μL, 5 eq) in DCM (1 mL) was stirred at 40° C. for 2 hours. The reaction mixture was diluted with a saturated brine solution (30 mL) and extracted with EA (20 mL*2). The combined organic layers were concentrated under reduced pressure to give a residue. The crude product was purified by prep-HPLC (column: CD04-Welch Utimate C18 150*25*7 um; mobile phase: [water(FA)-ACN]; gradient: 1%-22% B over 8 min). The eluent was concentrated under reduced pressure to remove ACN and then lyophilized to give the product. I-430 (31.56 mg, 31.67 μmol, 63.16% yield, 99.756% purity, FA salt) was obtained as a yellow solid. LCMS (Method D): Retention time: 0.242 min, (M+H)=948.4. ¹H NMR (400 MHz, METHANOL-d4) δ=8.59 (s, 1H), 8.41 (s, 2H), 8.37 (d, J=7.6 Hz, 1H), 8.32 (s, 1H), 7.99-7.92 (m, 1H), 7.91-7.80 (m, 2H), 7.50 (d, J=5.2 Hz, 1H), 7.39-7.34 (m, 1H), 7.19-7.14 (m, 1H), 4.73 (d, J=13.2 Hz, 1H), 4.38 (s, 2H), 3.90-3.72 (m, 8H), 3.69-3.59 (m, 4H), 3.52 (s, 3H), 3.45 (s, 1H), 3.35 (s, 1H), 3.24-3.17 (m, 5H), 3.15-3.05 (m, 2H), 3.01-2.78 (m, 10H), 2.68-2.58 (m, 6H), 2.15-1.95 (m, 5H), 1.88-1.73 (m, 2H), 1.41-1.23 (m, 5H). ¹⁹F NMR (376 MHz, METHANOL-d₄) δ=−120.659, −120.764.

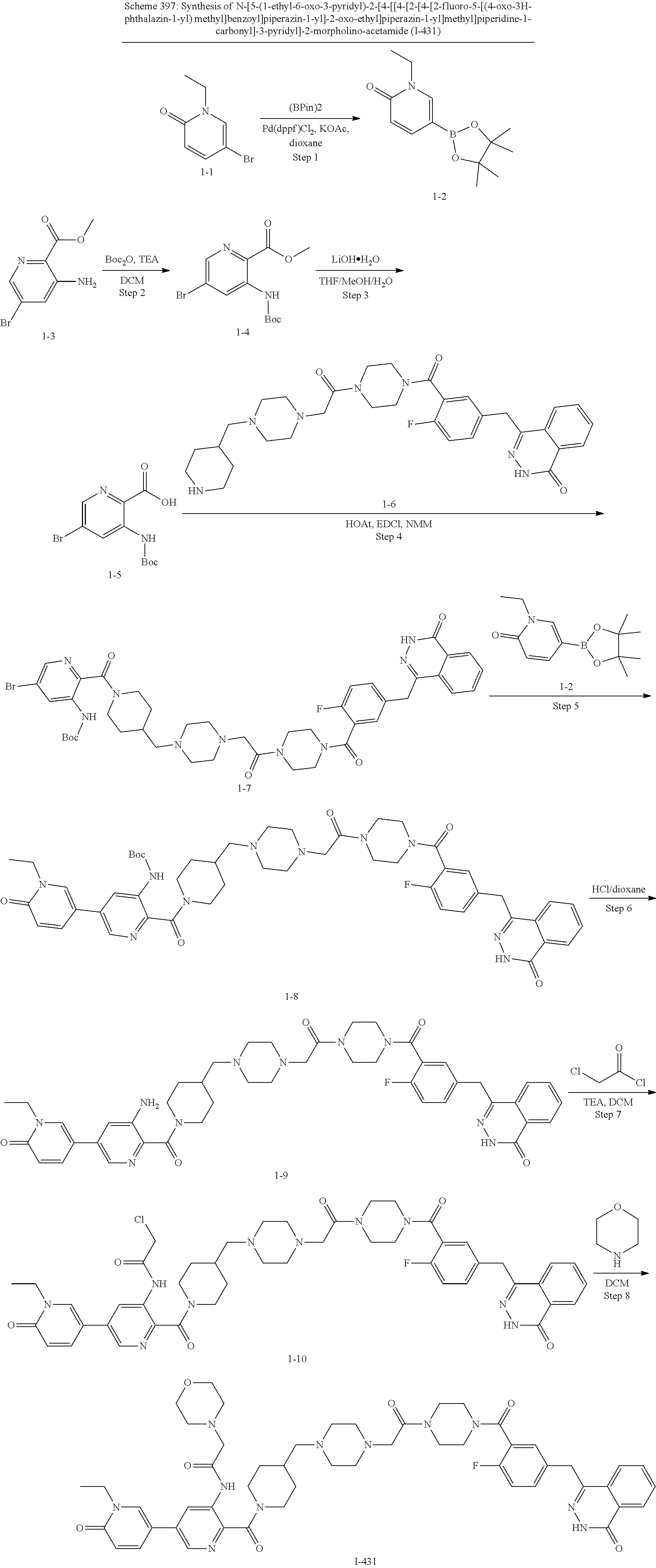

### Step 1: Synthesis of Intermediate 1-2.

To a mixture of Intermediate 1-1 (700 mg, 3.46 mmol, 1 eq) and Pin₂B₂ (1.32 g, 5.20 mmol, 1.5 eq) in dioxane (8 mL) was added Pd(dppf)Cl₂ (253.50 mg, 346.45 μmol, 0.1 eq), KOAc (1.02 g, 10.39 mmol, 3 eq), and then the mixture was stirred at 80° C. for 1 hour under N₂. The reaction mixture was diluted with water (50 mL) and extracted with EA (10 mL*3). The combined organic layers were concentrated under reduced pressure to give a residue. The residue was purified by flash silica gel chromatography ((ISCO; 40 g SepaFlash Silica Flash Column, Eluent of 0-50% Ethylacetate/Petroleum ether gradient @60 mL/min).). The eluent was concentrated under reduced pressure to give Intermediate 1-2 (500 mg, 1.75 mmol, 50.40% yield, 87% purity) as a white solid. LCMS (Method D): Retention time: 0.364 min, (M+H)=249.9.

### Step 2: Synthesis of Intermediate 1-4.

To a mixture of Intermediate 1-3 (2 g, 8.66 mmol, 1 eq) in DCM (20 mL) was added Boc₂O (2.27 g, 10.39 mmol, 2.39 mL, 1.2 eq), TEA (1.75 g, 17.31 mmol, 2.41 mL, 2 eq), DMAP (105.75 mg, 865.63 μmol, 0.1 eq), and then the mixture was stirred at 20° C. for 1 hour. The reaction mixture was diluted with water (100 mL) and extracted with EA (50 mL*3). The combined organic layers were concentrated under reduced pressure to give a residue. The residue was purified by flash silica gel chromatography (ISCO; 40 g SepaFlash Silica Flash Column, Eluent of 0˜50% Ethyl acetate/Petroleum ether gradient @80 mL/min). The eluent was concentrated under reduced pressure to give Intermediate 1-4 (2 g, 6.04 mmol, 69.77% yield) as a white solid. LCMS (Method D): Retention time: 0.477 min, (M+H)=330.8. ¹H NMR (400 MHz, DMSO-d6) δ=9.99 (s, 1H), 8.69 (d, J=2.0 Hz, 1H), 8.44 (d, J=2.0 Hz, 1H), 3.86 (s, 3H), 1.48 (s, 9H).

### Step 3: Synthesis of Intermediate 1-5.

To a mixture of Intermediate 1-4 (1.8 g, 5.44 mmol, 1 eq) in THF (20 mL), MeOH (10 mL), and H₂O (5 mL) was added LiOH·H₂O (684.27 mg, 16.31 mmol, 3 eq), then the mixture was stirred at 20° C. for 1 hour. The reaction mixture was concentrated under reduced pressure to remove THF, and 1 N HCl was added (about 10 mL) to adjust the pH to 3˜4, the mixture was extracted with EA (10 mL*3). The combined organic layers were concentrated under reduced pressure to give Intermediate 1-5 (2 g, crude) as a white solid. LCMS (Method D): Retention time: 0.418 min, (M+H)=260.8. ¹H NMR (400 MHz, DMSO-d6) δ=13.09 (s, 1H), 8.79 (s, 1H), 8.20 (s, 1H), 1.48 (s, 9H).

### Step 4: Synthesis of Intermediate 1-7

To a mixture of Intermediate 1-5 (150 mg, 472.98 μmol, 1 eq) and Intermediate 1-6 (278.92 mg, 472.98 μmol, 1 eq) in DMF (2 mL) was added HOAt (64.38 mg, 472.98 μmol, 66.16 μL, 1 eq), EDCI (272.01 mg, 1.42 mmol, 3 eq), NMM (239.20 mg, 2.36 mmol, 260.01 μL, 5 eq), and then the mixture was stirred at 20° C. for 1 hour. The reaction mixture was diluted with water (40 mL) and extracted with EA (15 mL*2). The combined organic layers were concentrated under reduced pressure to give Intermediate 1-7 (300 mg, crude) as a white solid. LCMS (Method D): Retention time: 0.362 min, (M+H)=888.3.

### Step 5: Synthesis of Intermediate 1-8.

To a mixture of Intermediate 1-7 (230 mg, 258.77 μmol, 1 eq) and Intermediate 1-2 (128.93 mg, 517.54 μmol, 2 eq) in dioxane (5 mL) and H₂O (1 mL) was added Pd(dppf)Cl₂ (18.93 mg, 25.88 μmol, 0.1 eq), K₃PO₄ (164.78 mg, 776.31 μmol, 3 eq), and then the mixture was stirred at 80° C. for 1 hour under N₂. The reaction mixture was concentrated under reduced pressure to give a residue. The residue was purified by flash silica gel chromatography (ISCO; 20 g SepaFlash Silica Flash Column, Eluent of 0˜50% ethyl acetate/Petroleum ether gradient @50 mL/min). The eluent was concentrated under reduced pressure to give Intermediate 1-8 (200 mg, 214.81 μmol, 83.01% yield) as a white solid. LCMS (Method D): Retention time: 0.352 min, (M+H)=931.4.

### Step 6: Synthesis of Intermediate 1-9.

A mixture of Intermediate 1-8 (60 mg, 64.44 μmol, 1 eq) in DCM (1 mL) was added HCl/dioxane (2 M, 161.11 μL, 5 eq), then the mixture was stirred at 20° C. for 5 min. The reaction mixture was concentrated under reduced pressure to give Intermediate 1-9 (60 mg, crude, HCl salt) as a white solid. LCMS (Method D): Retention time: 0.291 min, (M+H)=831.4.

### Step 7: Synthesis of Intermediate 1-10.

To a mixture of Intermediate 1-9 (60 mg, 69.17 μmol, 1 eq, HCl salt) in DCM (1 mL) was added TEA (21.00 mg, 207.51 μmol, 28.88 μL, 3 eq) and 2-chloroacetyl chloride (9.37 mg, 83.01 μmol, 6.61 μL, 1.2 eq), then the mixture was stirred at 0° C. for 5 min. The reaction mixture was quenched with water (20 mL) and extracted with DCM (5 mL*3). The combined organic layers were concentrated under reduced pressure to give Intermediate 1-10 (80 mg, crude) as a white solid. LCMS (Method D): Retention time: 0.308 min, (M+H)=907.3.

### Step 8: Synthesis of I-431

To a mixture of Intermediate 1-10 (80 mg, 88.16 μmol, 1 eq) in DCM (1 mL) was added morpholine (38.40 mg, 440.81 μmol, 38.79 μL, 5 eq), then the mixture was stirred at 40° C. for 1 hour. The reaction mixture was concentrated under reduced pressure to give a residue. The crude product was purified by reversed-phase HPLC (0.1% FA condition). The eluent was concentrated under reduced pressure to remove MeCN and then lyophilized to give I-431 (5.3 mg, 5.32 μmol, 6.03% yield, 96.10% purity) as a white solid. LCMS (Method D): Retention time: 0.270 min, (M+H)=958.4. ¹H NMR (400 MHz, METHANOL-d4) δ=8.76 (d, J=1.6 Hz, 1H), 8.56 (d, J=1.6 Hz, 1H), 8.37 (d, J=7.6 Hz, 1H), 8.18 (d, J=2.0 Hz, 1H), 7.97-7.81 (m, 4H), 7.50 (s, 1H), 7.40-7.31 (m, 1H), 7.21-7.13 (m, 1H), 6.68 (d, J=9.6 Hz, 1H), 4.73 (d, J=12.4 Hz, 1H), 4.38 (s, 2H), 4.19-4.11 (m, 2H), 3.88-3.85 (m, 2H), 3.83-3.80 (m, 4H), 3.75 (s, 1H), 3.68 (s, 2H), 3.52 (s, 2H), 3.44 (s, 1H), 3.37 (s, 2H), 3.22-3.18 (m, 4H), 3.15 (s, 1H), 2.97-2.89 (m, 1H), 2.74 (s, 7H), 2.62 (s, 4H), 2.54-2.46 (m, 2H), 2.04-1.93 (m, 2H), 1.77 (d, J=12.0 Hz, 1H), 1.44-1.37 (m, 3H), 1.35-1.23 (m, 2H). ¹⁹F NMR (376 MHz, METHANOL-d4) δ=−120.77.

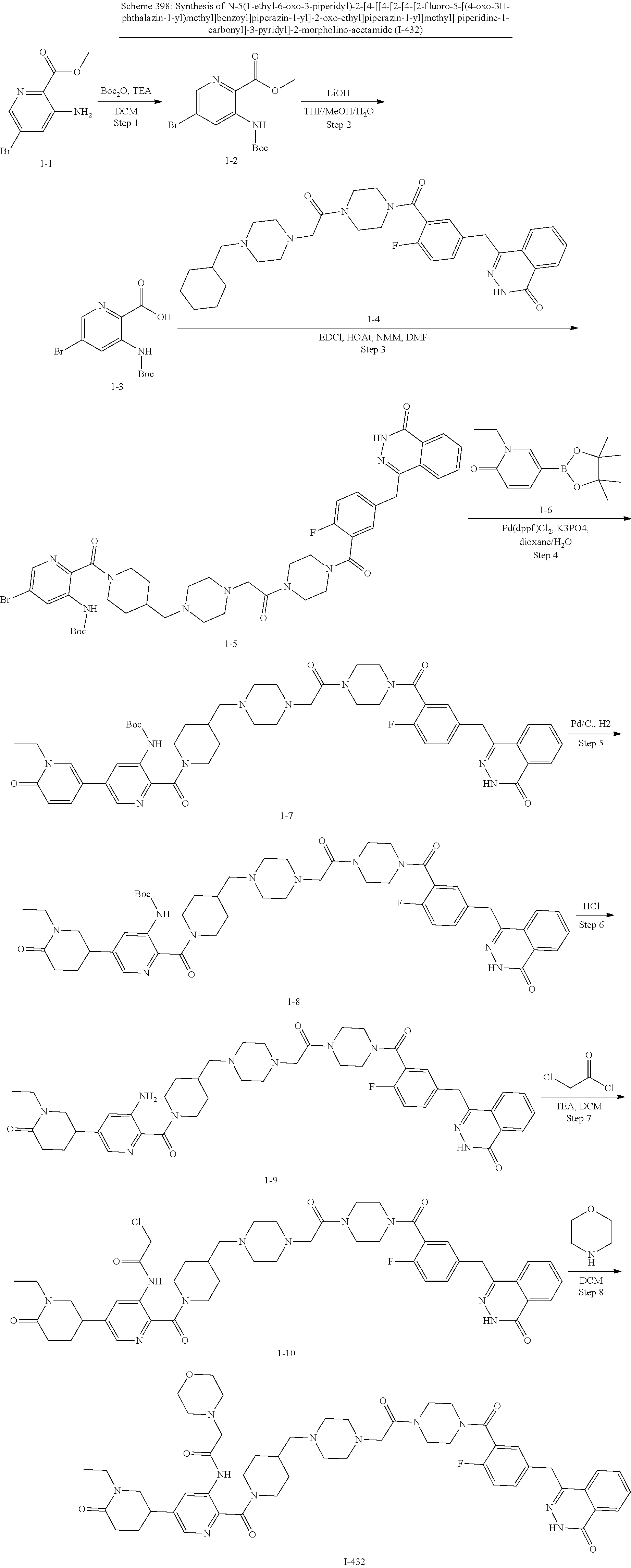

### Step 1: Synthesis of Intermediate 1-2.

To a mixture of Intermediate 1-1 (5 g, 21.64 mmol, 1 eq) in DCM (50 mL) was added Boc₂O (5.67 g, 25.97 mmol, 5.97 mL, 1.2 eq), TEA (4.38 g, 43.28 mmol, 6.02 mL, 2 eq), and DMAP (264.38 mg, 2.16 mmol, 0.1 eq), then the mixture was stirred at 20° C. for 1 hour. The reaction mixture was diluted with water (100 mL) and extracted with EA (50 mL*3). The combined organic layers were concentrated under reduced pressure to give a residue. The residue was purified by flash silica gel chromatography (ISCO; 40 g SepaFlash Silica Flash Column, Eluent of 0˜50% Ethyl acetate/Petroleum ether gradient @80 mL/min). The eluent was concentrated under reduced pressure to give Intermediate 1-2 (5 g, 15.10 mmol, 69.77% yield) as a white solid. LCMS (Method D): Retention time: 0.475 min, (M+H)=216.8.

### Step 2: Synthesis of Intermediate 1-3.

To a mixture of Intermediate 1-2 (5 g, 15.10 mmol, 1 eq) in THF (20 mL), MeOH (10 mL), and H₂O (5 mL) was added LiOH·H₂O (696.94 mg, 16.61 mmol, 1.1 eq), then the mixture was stirred at 20° C. for 1 hour. The reaction mixture was concentrated under reduced pressure to remove THF, and 1 N HCl was added (about 10 mL) to adjust the pH to 3˜4, and the mixture was extracted with EA (10 mL*3). The combined organic layers were concentrated under reduced pressure to give Intermediate 1-3 (4 g, crude) as a white solid. LCMS (Method D): Retention time: 0.431 min, (M+H)=260.8.

### Step 3: Synthesis of Intermediate 1-5.

To a mixture of Intermediate 1-3 (200 mg, 630.65 μmol, 1 eq) and Intermediate 1-4 (371.89 mg, 630.65 μmol, 1 eq) in DMF (3 mL) was added HOAt (85.84 mg, 630.65 μmol, 88.22 μL, 1 eq), EDCI (362.69 mg, 1.89 mmol, 3 eq), and NMM (318.94 mg, 3.15 mmol, 346.67 μL, 5 eq), then the mixture was stirred at 20° C. for 1 hour. The reaction mixture was diluted with water (100 mL) and extracted with EA (20 mL*3). The combined organic layers were concentrated under reduced pressure to give Intermediate 1-5 (300 mg, 337.53 μmol, 53.52% yield) as a white solid.

### Step 4: Synthesis of Intermediate 1-7.

To a mixture of Intermediate 1-5 (300 mg, 337.53 μmol, 1 eq) and Intermediate 1-6 (252.25 mg, 1.01 mmol, 3 eq) in dioxane (2 mL) and H₂O (0.4 mL) was added Pd(dppf)Cl₂ (24.70 mg, 33.75 μmol, 0.1 eq) and K₃PO₄ (214.94 mg, 1.01 mmol, 3 eq), and then the mixture was stirred at 80° C. for 1 hour under N₂. The reaction mixture was concentrated under reduced pressure to give a residue. The residue was purified by flash silica gel chromatography (ISCO; 20 g SepaFlash Silica Flash Column, Eluent of 0˜60% Ethyl acetate/Petroleum ether gradient @60 mL/min). The eluent was concentrated under reduced pressure to give Intermediate 1-7 (200 mg, 214.81 μmol, 63.64% yield) as a white solid. LCMS (Method D): Retention time: 0.350 min, (M+H)=931.5.

### Step 5: Synthesis of Intermediate 1-8.

To a mixture of Intermediate 1-7 (150 mg, 161.11 μmol, 1 eq) in DMF (1 mL) was added Pd/C (1.71 mg, 1.61 μmol, 10% purity, 0.01 eq). The suspension was degassed and purged with N₂ 3 times, and then the mixture was degassed and purged with H₂ (15 psi) 3 times, then the mixture was stirred at 100° C. for 1 hour under H₂ (324.76 g, 161.11 μmol, 1 eq) atmosphere (15 psi). The reaction mixture was filtered and concentrated under reduced pressure to give Intermediate 1-8 (120 mg, 128.33 μmol, 79.66% yield) as a white solid.

### Step 6: Synthesis of Intermediate 1-9.

A mixture of Intermediate 1-8 (90 mg, 96.25 μmol, 1 eq) in DCM (1 mL) was added HCl/dioxane (2 M, 240.62 μL, 5 eq) and it was stirred at 20° C. for 5 min. The reaction mixture was concentrated under reduced pressure to give Intermediate 1-9 (80 mg, crude, HCl salt) as a white solid.

### Step 7: Synthesis of Intermediate 1-10.

To a mixture of Intermediate 1-9 (60.28 mg, 69.17 μmol, 1 eq, HCl salt) in DCM (1 mL) was added TEA (21.00 mg, 207.51 μmol, 28.88 μL, 3 eq) and 2-chloroacetyl chloride (7.81 mg, 69.17 mol, 5.51 μL, 1 eq), then the mixture was stirred at 0° C. for 5 min. The reaction mixture was quenched with water (20 mL) and extracted with DCM (5 mL*3). The combined organic layers were concentrated under reduced pressure to give Intermediate 1-10 (60 mg, 65.83 μmol, 95.17% yield) as a white solid. LCMS (Method D): Retention time: 0.300 min, (M+H)=911.4.

### Step 8: Synthesis of I-432

To a mixture of Intermediate 1-10 (60 mg, 65.83 μmol, 1 eq) in DCM (1 mL) was added morpholine (28.67 mg, 329.14 μmol, 28.96 μL, 5 eq), then the mixture was stirred at 40° C. for 1 hour. The reaction mixture was concentrated under reduced pressure to give a residue. The crude product was purified by reversed-phase HPLC (column: CD04-Welch Utimate C18 150*25*7 um; mobile phase: [water(FA)-ACN]; gradient: 1%-28% B over 8 min). The eluent was concentrated under reduced pressure to remove MeCN and then lyophilized to give I-432 (10.14 mg, 9.99 μmol, 15.17% yield, 99.29% purity, FA salt) as a white solid. LCMS (Method D): Retention time: 0.288 min, (M+H)=962.5. SFC: Rt=6.270 min, Rt=8.116 min. ¹H NMR (400 MHz, METHANOL-d4) δ=8.58 (d, J=1.6 Hz, 1H), 8.38 (s, 1H), 8.36-8.31 (m, 2H), 7.99-7.92 (m, 1H), 7.91-7.80 (m, 2H), 7.55-7.47 (m, 1H), 7.39-7.33 (m, 1H), 7.19-7.14 (m, 1H), 4.72 (d, J=12.8 Hz, 1H), 4.39 (s, 2H), 3.83-3.73 (m, 7H), 3.67 (s, 2H), 3.58-3.42 (m, 8H), 3.37-3.32 (m, 2H), 3.30-3.25 (m, 1H), 3.19-3.09 (m, 3H), 3.02-2.76 (m, 9H), 2.69-2.57 (m, 6H), 2.55-2.46 (m, 2H), 2.16-1.94 (m, 4H), 1.76 (d, J=12.4 Hz, 1H), 1.39-1.23 (m, 2H), 1.18-1.14 (m, 3H). ¹⁹F NMR (376 MHz, METHANOL-d4) δ=−120.70.

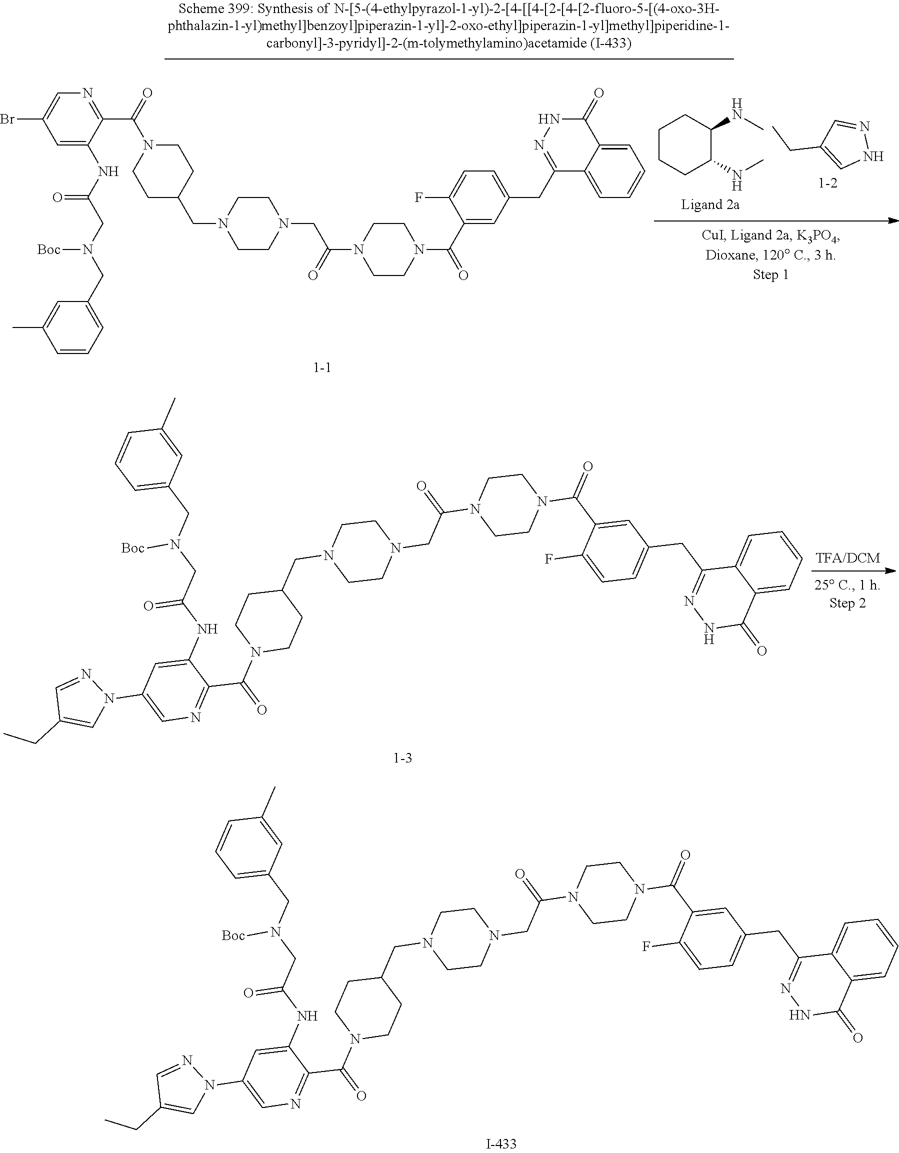

### Step 1: Synthesis of Intermediate 1-3

To a solution of Intermediate 1-2 (12.82 mg, 133.33 μmol, 1 eq) and Intermediate 1-1 (140 mg, 133.33 μmol, 1 eq) in dioxane (2 mL) was added K₃PO₄ (70.75 mg, 333.33 μmol, 2.5 eq), rac-(1R,2R)—N1, N2-dimethylcyclohexane-1,2-diamine (1.90 mg, 13.33 μmol, 0.1 eq) and CuI (2.54 mg, 13.33 μmol, 0.1 eq). The suspension was degassed and purged with N₂ 3 times. The mixture was stirred at 100° C. for 2 hr under N₂. The reaction mixture was diluted with water (30 mL) and extracted with ethyl acetate (10 mL×3). The combined organic layers were washed with brine (10 mL×3) and dried over sodium sulfate, filtered and concentrated under reduced pressure to give a residue. The crude product was purified by reversed-phase HPLC (0.1% FA condition). The eluent was concentrated under reduced pressure to remove ACN and then lyophilized to give Intermediate 1-3 (100 mg, 55.95 μmol, 41.96% yield, 59.6% purity) as a yellow solid. LCMS (Method D): Rt: 0.427 min, (M+H)=1065.4.

### Step 2: Synthesis of I-433

To a solution of Intermediate 1-3 (90 mg, 84.49 μmol, 1 eq) in DCM (2 mL) was added TFA (614.00 mg, 5.39 mmol, 0.4 mL, 63.74 eq). The mixture was stirred at 25° C. for 1 hr. The mixture was concentrated to give a residue. The crude product was purified by reversed-phase chromatography. The eluent was concentrated under reduced pressure to remove ACN and then lyophilized to give I-433 (12.01 mg, 11.34 μmol, 13.42% yield, 91.128% purity) as an off-white solid. LCMS (Method F): Rt: 0.637 min, (M+H)=965.7. ¹H NMR (400 MHz, METHANOL-d4) δ=8.96 (s, 1H), 8.77 (d, J=2.0 Hz, 1H), 8.37 (d, J=7.6 Hz, 1H), 8.15 (s, 1H), 7.94 (d, J=4.0 Hz, 1H), 7.91-7.75 (m, 2H), 7.67 (s, 1H), 7.55-7.45 (m, 1H), 7.42-7.35 (m, 1H), 7.28 (s, 1H), 7.24-7.12 (m, 3H), 7.09-7.07 (m, 1H), 4.72-4.50 (m, 2H), 4.39 (s, 2H), 3.82-3.69 (m, 6H), 3.66 (d, J=2.4 Hz, 1H), 3.53 (d, J=16.4 Hz, 3H), 3.44-3.36 (m, 2H), 3.27 (s, 2H), 3.23-3.06 (m, 3H), 2.89-2.85 (m, 1H), 2.65-2.41 (m, 10H), 2.34 (s, 3H), 2.25-2.16 (m, 2H), 1.91-1.80 (m, 2H), 1.78-1.71 (m, 1H), 1.28-1.16 (m, 3H). ¹⁹F NMR (400 MHz, METHAN OL-d4) δ=−120.810.

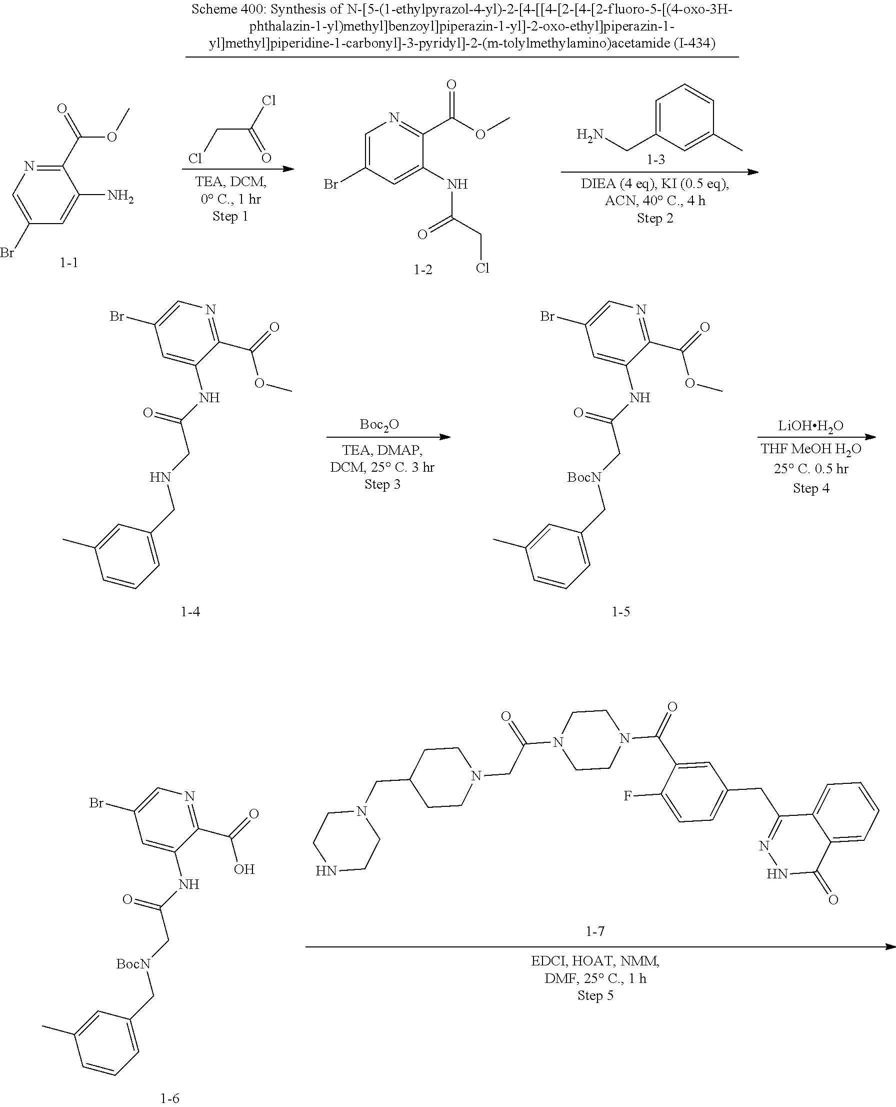

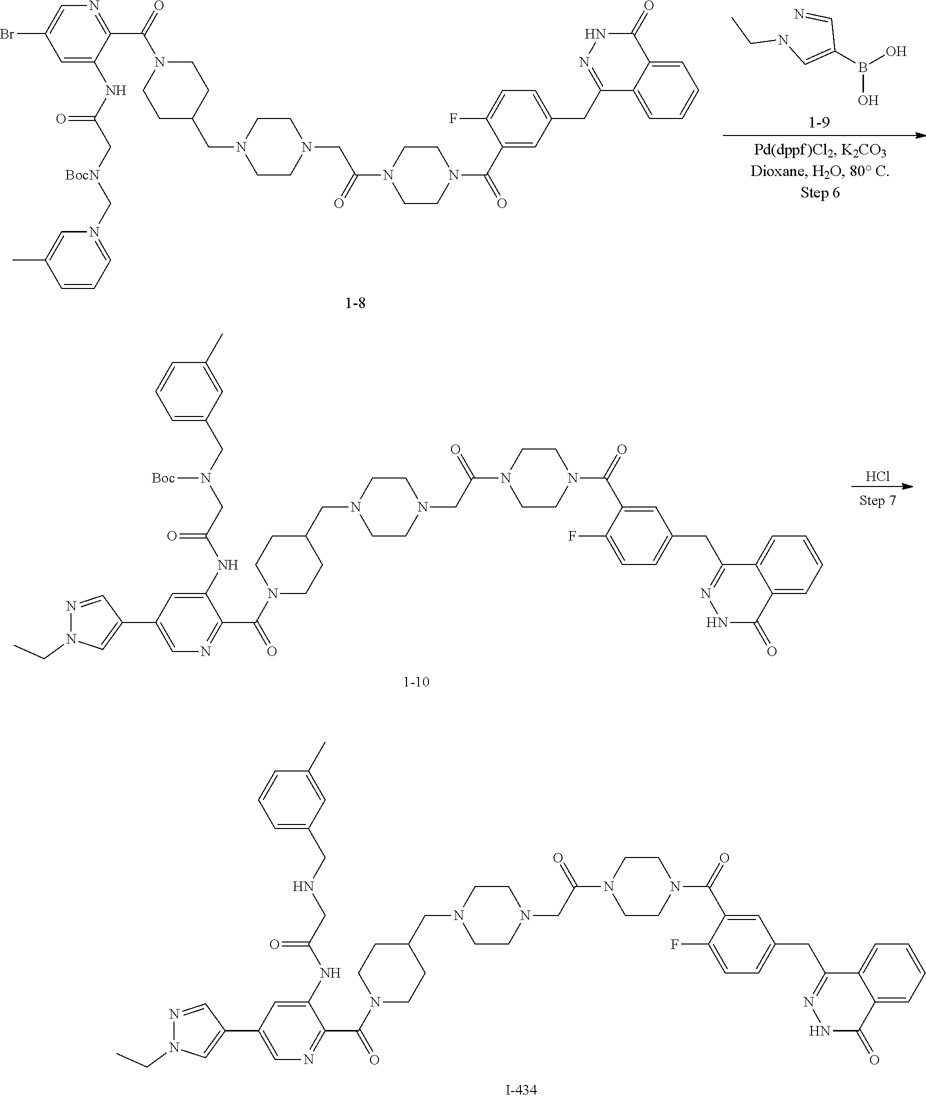

### Step 1: Synthesis of Intermediate 1-2

To a solution of Intermediate 1-1 (30 g, 129.84 mmol, 1 eq) and TEA (26.28 g, 259.69 mmol, 36.15 mL, 2 eq) in DCM (100 mL) was added 2-chloroacetyl chloride (22.00 g, 194.77 mmol, 15.51 mL, 1.5 eq) at 25° C. The mixture was stirred at 25° C. for 0.5 hr. The mixture was concentrated to give a residue. The residue was triturated with DCM (150 mL) and filtered to afford Intermediate 1-2 (25 g, crude) as a white solid. LCMS (Method D): Rt: 0.352 min, (M+H)=306.8.

### Step 2: Synthesis of Intermediate 1-4

To a solution of Intermediate 1-2 (5.08 g, 16.50 mmol, 1 eq) and Intermediate 1-3 (2 g, 16.50 mmol, 2.07 mL, 1 eq) in ACN (50 mL) was added DIEA (3.20 g, 24.76 mmol, 4.31 mL, 1.5 eq) and KI (1.37 g, 8.25 mmol, 0.5 eq). The mixture was stirred at 60° C. for 1 hr. The mixture was concentrated to give Intermediate 1-4 (6.5 g, crude) as a brown liquid. LCMS (Method D): Rt: 0.291 min, (M+H)=391.9.

### Step 3: Synthesis of Intermediate 1-5

To a solution of Intermediate 1-4 (6.5 g, 16.57 mmol, 1 eq) in ACN (80 mL) was added Boc₂O (4.34 g, 19.89 mmol, 4.57 mL, 1.2 eq), TEA (3.35 g, 33.14 mmol, 4.61 mL, 2 eq) and DMAP (202.45 mg, 1.66 mmol, 0.1 eq). The mixture was stirred at 25° C. for 1 hr. The mixture was concentrated to give a residue. The reaction mixture was diluted with water (120 mL) and extracted with ethyl acetate (40 mL×3). The combined organic layers were washed with brine (40 mL×3) and dried over sodium sulfate, filtered and concentrated under reduced pressure to give a residue. The residue was purified by flash silica gel chromatography (ISCO; 80 g SepaFlash Silica Flash Column, Eluent of 20-60% Ethylacetate/Petroleum ether gradient @100 mL/min). The eluent was concentrated under reduced pressure to give Intermediate 1-5 (2.2 g, 3.81 mmol, 22.99% yield, 85.25% purity) as a yellow solid. LCMS (Method D): Rt: 0.517 min, (M−H)=492.0. ¹H NMR (400 MHz, DMSO-d6) δ=11.00-10.55 (m, 1H), 9.04-8.78 (m, 1H), 8.54 (d, J=2.0 Hz, 1H), 7.25-7.13 (m, 1H), 7.11-6.99 (m, 3H), 4.47 (s, 2H), 3.98 (d, J=9.6 Hz, 2H), 3.91-3.86 (m, 3H), 2.25 (s, 3H), 1.45-1.29 (m, 9H).

### Step 4: Synthesis of Intermediate 1-6

To a solution of Intermediate 1-5 (2.2 g, 4.47 mmol, 1 eq) in THF (10 mL), MeOH (12 mL) and H₂O (2.5 mL) was added LiOH·H₂O (562.51 mg, 13.40 mmol, 3 eq). The mixture was stirred at 50° C. for 1 hr. The mixture was concentrated to give a residue. The reaction mixture was diluted with water (60 mL). Then the mixture was adjusted to pH=6-7 with saturated citric acid aqueous solution. Then the reaction mixture was extracted with DCM (20 mL×3). The combined organic layers were washed with brine (20 mL×3) and dried over sodium sulfate, filtered and concentrated under reduced pressure to give a residue. The crude product was purified by reversed-phase HPLC (0.1% FA condition). The eluent was concentrated under reduced pressure to remove ACN and then lyophilized to give Intermediate 1-6 (0.8 g, 1.67 mmol, 37.43% yield, 100% purity) as a yellow solid. LCMS (Method D): Rt: 0.482 min, (M+H-100)=377.9. ¹H NMR (400 MHz, DMSO-d6) δ=11.46 (s, 1H), 9.11 (s, 1H), 8.52 (d, J=1.6 Hz, 1H), 7.29-7.15 (m, 1H), 7.13-7.01 (m, 3H), 4.50 (s, 2H), 4.09-3.90 (m, 3H), 2.26 (s, 3H), 1.43-1.29 (m, 9H).

### Step 5: Synthesis of Intermediate 1-8

To a solution of Intermediate 1-6 (230 mg, 480.83 μmol, 1 eq) in DMF (5 mL) was added EDCI (276.53 mg, 1.44 mmol, 3 eq), HOAt (65.45 mg, 480.83 μmol, 67.26 μL, 1 eq) and NMM (243.17 mg, 2.40 mmol, 264.32 μL, 5 eq). Then Intermediate 1-7 (301.08 mg, 480.83 μmol, 1 eq, HCl salt) was added into the mixture and it was stirred at 25° C. for 1 hr. The reaction mixture was diluted with water (60 mL) and extracted with ethyl acetate (20 mL×3). The combined organic layers were washed with brine (20 mL×3) and dried over sodium sulfate, filtered and concentrated under reduced pressure to give a residue. The crude product was purified by reversed-phase HPLC (0.1% FA condition). The eluent was concentrated under reduced pressure to remove ACN and then lyophilized to give Intermediate 1-8 (300 mg, 237.11 μmol, 49.31% yield, 82.99% purity) as a brown solid. LCMS (Method D): Rt: 0.421 min, (M+H)=1049.3.

### Step 6: Synthesis of Intermediate 1-10

To a solution of Intermediate 1-9 (18.66 mg, 133.33 μmol, 1 eq) and Intermediate 1-8 (140 mg, 133.33 μmol, 1 eq) in dioxane (2 mL) and H₂O (0.5 mL) was added Pd(dppf)Cl₂ (9.76 mg, 13.33 μmol, 0.1 eq) and K₂CO₃ (55.28 mg, 399.99 μmol, 3 eq). The suspension was degassed and purged with N₂ 3 times. The mixture was stirred at 90° C. for 1 hr under N₂. The reaction mixture was diluted with water (60 mL) and extracted with ethyl acetate (20 mL×3). The combined organic layers were washed with brine (20 mL×3) and dried over sodium sulfate, filtered and concentrated under reduced pressure to give a residue. The residue was purified by flash silica gel chromatography (ISCO; 12 g SepaFlash Silica Flash Column, Eluent of 0˜10% Methanol/Ethylacetate ether gradient @50 mL/min). The eluent was concentrated under reduced pressure to give Intermediate 1-10 (100 mg, 50.69 μmol, 38.02% yield, 54% purity) as a brown solid. LCMS (Method D): Rt: 0.404 min, (M+H)=1065.7.

### Step 7: Synthesis of I-434

To a solution of Intermediate 1-10 (100 mg, 93.88 μmol, 1 eq) in DCM (1 mL) was added TFA (460.50 mg, 4.04 mmol, 0.3 mL). The mixture was stirred at 25° C. for 1 hr. The mixture was concentrated to give a residue. The crude product was purified by reversed-phase HPLC (0.1% TFA condition). The eluent was concentrated under reduced pressure to remove ACN and then lyophilized to give I-434 (68.38 mg, 62.45 μmol, 66.52% yield, 98.55% purity, TFA salt) as a white solid. LCMS (Method D): Rt: 0.340 min, (M+H)=965.6. ¹H NMR (400 MHz, METHANOL-d4) δ=8.64 (d, J=1.2 Hz, 1H), 8.37-8.35 (m, 1H), 8.26-8.14 (m, 2H), 7.98-7.91 (m, 2H), 7.90-7.78 (m, 2H), 7.50 (d, J=4.8 Hz, 1H), 7.41-7.26 (m, 5H), 7.21-7.12 (m, 1H), 4.86-4.75 (m, 2H), 4.63 (d, J=13.2 Hz, 1H), 4.38 (s, 2H), 4.30-4.21 (m, 4H), 4.00 (s, 2H), 3.88-3.73 (m, 5H), 3.69 (s, 1H), 3.60 (s, 1H), 3.53 (d, J=4.4 Hz, 1H), 3.45 (s, 1H), 3.41-3.32 (m, 4H), 3.24-3.12 (m, 5H), 2.97 (d, J=6.0 Hz, 2H), 2.93-2.85 (m, 1H), 2.39 (s, 3H), 2.16 (s, 1H), 1.95 (d, J=11.6 Hz, 1H), 1.77 (d, J=12.0 Hz, 1H), 1.60-1.47 (m, 4H), 1.45-1.34 (m, 1H). ¹⁹F NMR (400 MHz, METHAN OL-d4) δ=−77.09, −120.682.

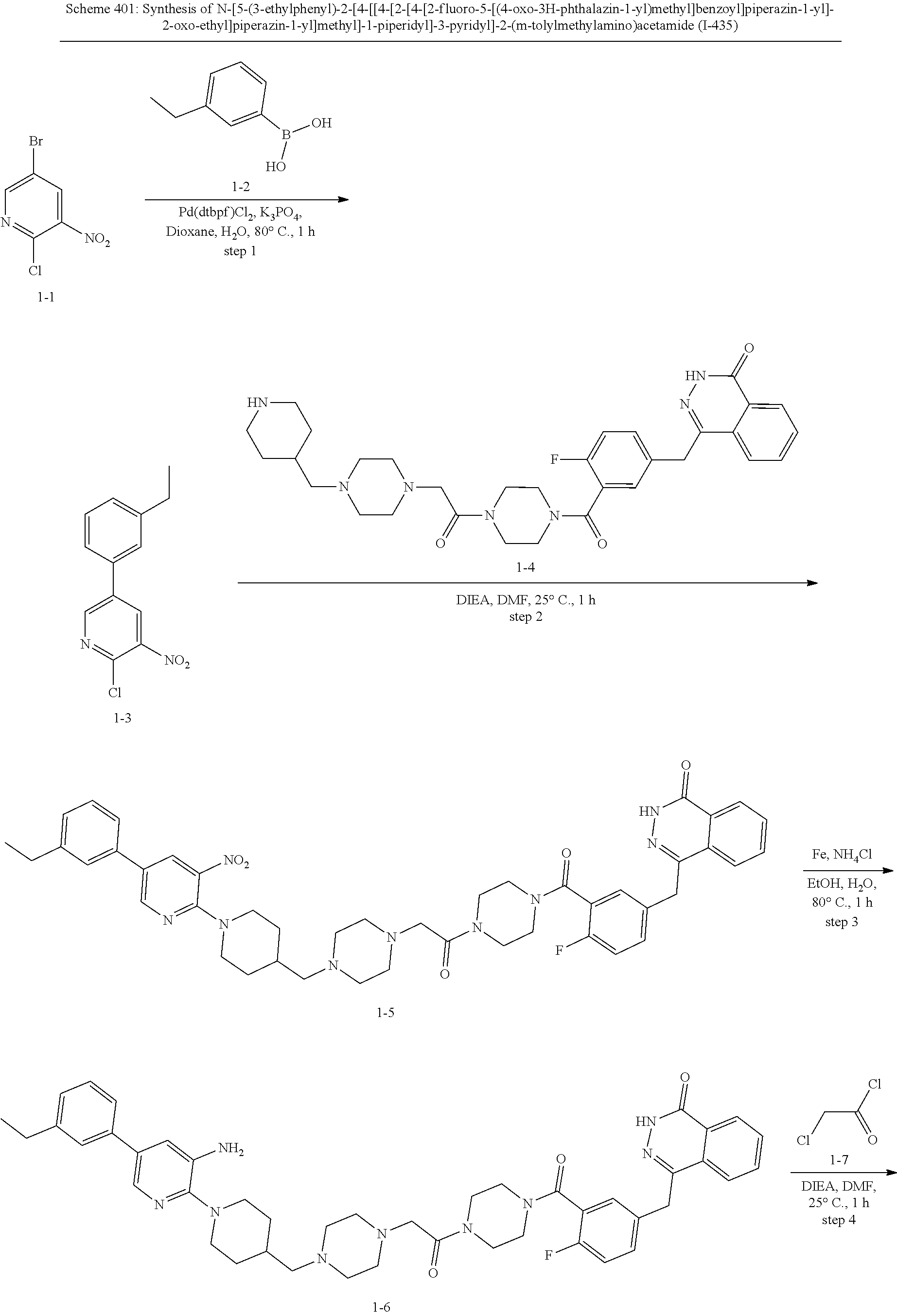

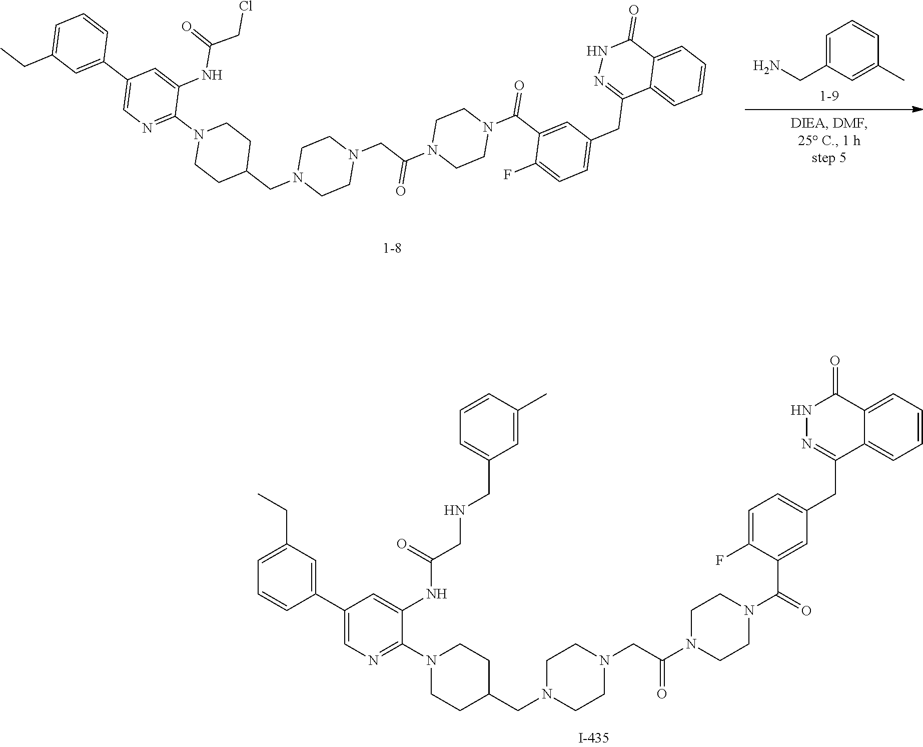

### Step 1: Synthesis of Intermediate 1-3

A mixture of intermediate 1-1 (1 g, 4.21 mmol, 1 eq), intermediate 1-2 (758.00 mg, 5.05 mmol, 1.2 eq), K₃PO₄ (2.68 g, 12.63 mmol, 3 eq) and Pd(dtbpf)Cl₂ (274.49 mg, 421.16 μmol, 0.1 eq) in dioxane (10 mL) and H₂O (2 mL) was degassed and purged with N₂ 3 times, and then the mixture was stirred at 80° C. for 1 hr under N₂ atmosphere. The reaction was poured into H₂O (10 mL) and extracted with DCM (15 mL*3). The combined organic layers were dried over Na₂SO₄, filtered and concentrated under reduced pressure to give the crude product. The residue was purified by flash silica gel chromatography (ISCO; 40 g SepaFlash Silica Flash Column, Eluent of 0˜100% Ethylacetate/Petroleum ethergradient @40 mL/min) and concentrated under reduced pressure to give a residue. Intermediate 1-3 (1.1 g, 3.22 mmol, 76.56% yield, 77% purity) was obtained as a brown solid. LCMS (Method D): Rt=0.495 min, (M+H)=263.1.

### Step 2: Synthesis of Intermediate 1-5

To a solution of intermediate 1-3 (500 mg, 1.47 mmol, 1 eq) and intermediate 1-4 (1.01 g, 1.61 mmol, 1.1 eq, HCl) in DMF (5 mL) was added DIEA (568.25 mg, 4.40 mmol, 765.84 μL, 3 eq). The mixture was stirred at 25° C. for 1 hr. The reaction mixture was concentrated under reduced pressure to give a residue. The residue was purified by flash silica gel chromatography (ISCO; 40 g SepaFlash Silica Flash Column, Eluent of 0-65% Ethyl acetate/Methanol gradient @60 mL/min) and concentrated under reduced pressure to give a residue. Intermediate 1-5 (1.3 g, 1.43 mmol, 97.84% yield, 90% purity) was obtained as a brown solid. LCMS (Method D): Rt=0.424 min, (M+H)=816.3.

### Step 3: Synthesis of Intermediate 1-6

To a solution of intermediate 1-5 (500 mg, 612.80 μmol, 1 eq) in EtOH (5 mL) and H₂O (1 mL) was added Fe (102.66 mg, 1.84 mmol, 3 eq) and NH₄Cl (163.90 mg, 3.06 mmol, 5 eq). The mixture was stirred at 80° C. for 1 hr. The reaction mixture was diluted with MeOH (3 mL*3), filtered and concentrated under reduced pressure to give a residue. The residue was purified by flash silica gel chromatography (ISCO; 40 g SepaFlash Silica Flash Column, Eluent of 0˜60% Ethyl acetate/Methanol gradient @60 mL/min) and concentrated under reduced pressure to give a residue. Intermediate 1-6 (200 mg, 234.11 μmol, 38.20% yield, 92% purity) was obtained as a brown oil. LCMS (Method D): Rt=0.330 min, (M+H)=786.4.

### Step 4: Synthesis of Intermediate 1-8

To a solution of intermediate 1-6 (100 mg, 127.23 μmol, 1 eq) in DMF (1 mL) was added DIEA (32.89 mg, 254.47 μmol, 44.32 μL, 2 eq) and intermediate 1-7 (17.24 mg, 152.68 μmol, 12.16 μL, 1.2 eq). The mixture was stirred at 25° C. for 1 hr. The reaction mixture was concentrated under reduced pressure to give a residue. The residue was purified by flash silica gel chromatography (ISCO; 12 g SepaFlash Silica Flash Column, Eluent of 0˜50% Ethyl acetate/Methanol gradient @40 mL/min). Intermediate 1-8 (85 mg, 82.79 μmol, 65.07% yield, 84% purity) was obtained as a light yellow oil. LCMS (Method D): Rt=0.445 min, (M+H)=862.4.

### Step 5: Synthesis of I-435

To a solution of intermediate 1-8 (65 mg, 75.37 μmol, 1 eq) in DMF (0.5 mL) was added DIEA (19.48 mg, 150.74 μmol, 26.26 μL, 2 eq) and intermediate 1-9 (10.96 mg, 90.44 μmol, 11.35 μL, 1.2 eq). The mixture was stirred at 25° C. for 1 hr. The reaction mixture was concentrated under reduced pressure to give a residue. The crude product was purified by reversed-phase HPLC (0.1% FA condition) followed by lyophilization to give a residue. I-435 (8 mg, 8.05 μmol, 10.69% yield, 100% purity, FA salt) was obtained as a white solid. LCMS (Method D): Rt=0.360 min, (M+H)=947. ¹H NMR (400 MHz, METHANOL-d4) δ=8.81 (s, 1H), 8.50-8.44 (m, 1H), 8.38 (d, J=7.2 Hz, 1H), 8.25 (d, J=2.4 Hz, 1H), 7.99-7.92 (m, 1H), 7.91-7.81 (m, 2H), 7.54-7.46 (m, 1H), 7.43 (s, 1H), 7.41-7.35 (m, 3H), 7.25-7.20 (m, 4H), 7.19-7.14 (m, 1H), 7.09-7.04 (m, 1H), 4.39 (s, 2H), 3.85 (s, 2H), 3.80 (d, J=3.2 Hz, 1H), 3.78-3.73 (m, 1H), 3.73-3.67 (m, 2H), 3.60-3.50 (m, 3H), 3.47 (s, 3H), 3.35 (d, J=2.0 Hz, 3H), 2.88-2.81 (m, 2H), 2.76-2.68 (m, 3H), 2.68-2.44 (m, 8H), 2.31 (d, J=3.2 Hz, 3H), 2.27-2.18 (m, 2H), 1.88-1.81 (m, 2H), 1.78-1.67 (m, 1H), 1.45-1.35 (m, 2H), 1.28 (t, J=7.6 Hz, 3H). ¹⁹F NMR (376 MHz, METHAN OL-d4) δ=−120.800.

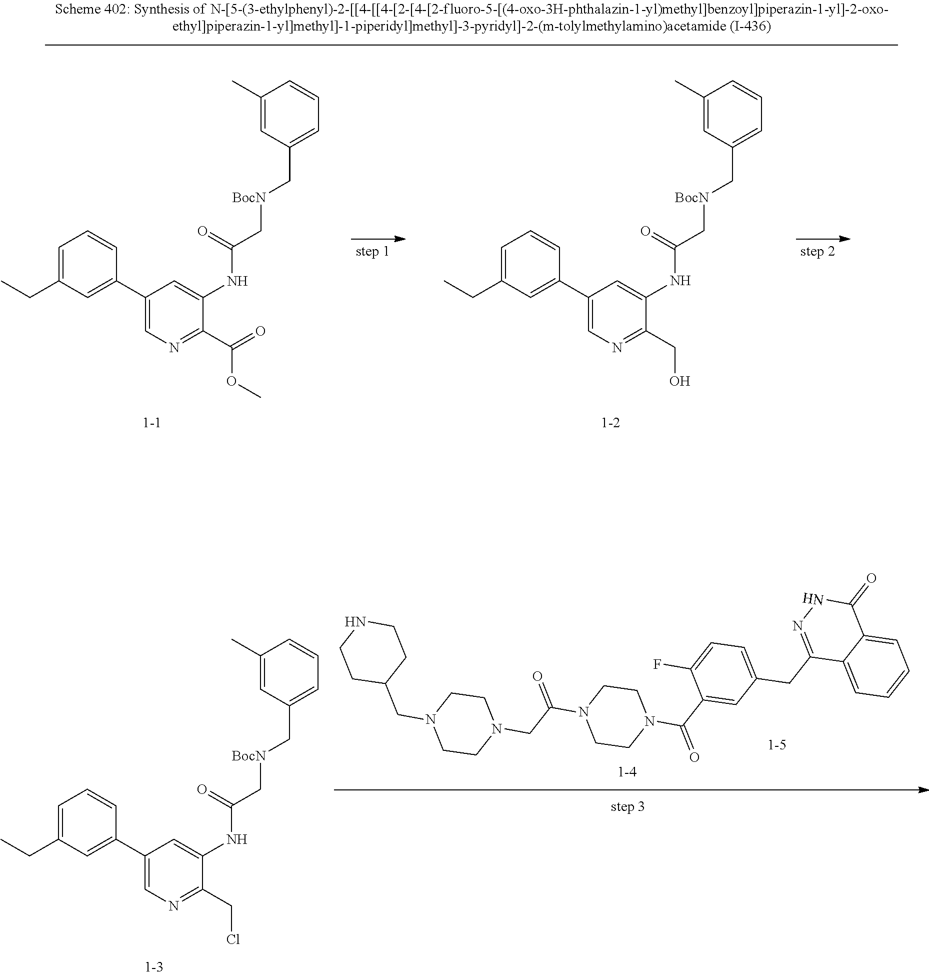

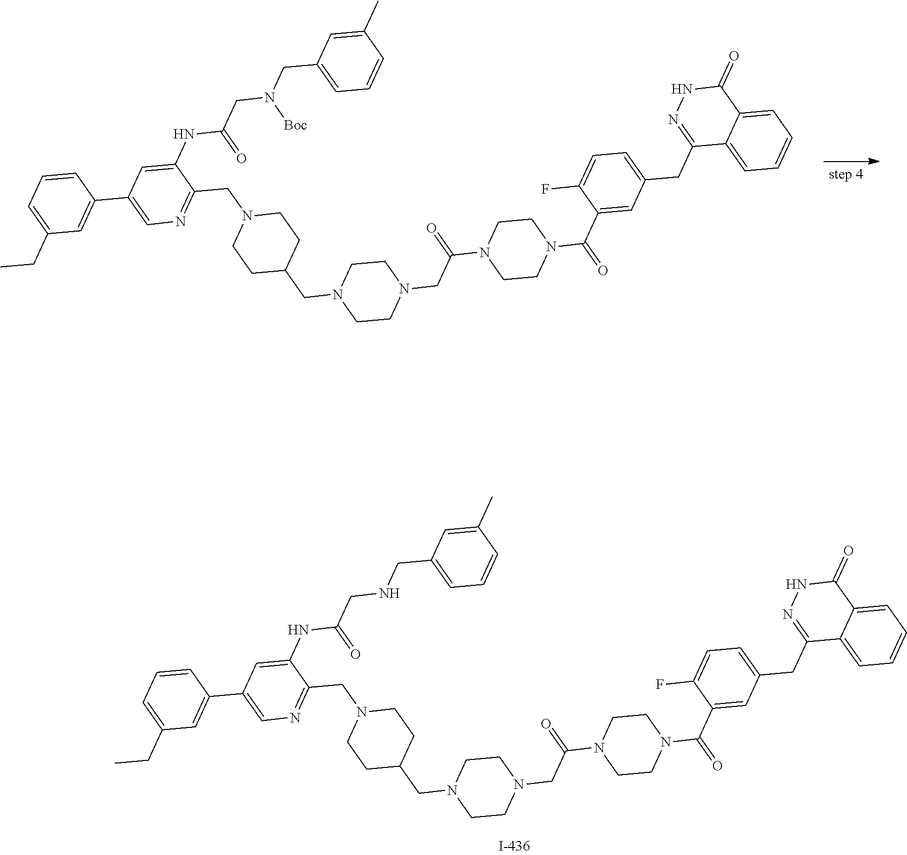 [SMILES: CCc1cccc(-c2cnc(CN3CCC(CN4CCN(CC(=O)N5CCN(C(=O)c6cc(Cc7n[nH]c(=O)c8ccccc78)ccc6F)CC5)CC4)CC3)c(NC(=O)CN(Cc3cccc(C)c3)C(=O)OC(C)(C)C)c2)c1 ; CCc1cccc(-c2cnc(CN3CCC(CN4CCN(CC(=O)N5CCN(C(=O)c6cc(Cc7n[nH]c(=O)c8ccccc78)ccc6F)CC5)CC4)CC3)c(NC(=O)CNCc3cccc(C)c3)c2)c1]

### Step 1: Synthesis of Intermediate 1-2

To a solution of Intermediate 1-1 (0.3 g, 579.58 μmol, 1 eq) in THF (3 mL) was added LAH (2.5 M, 1.62 mL, 7 eq) at 0° C. under N₂ atmosphere and the mixture was stirred at 25° C. for 2 hr under N₂ atmosphere. The reaction was quenched with Na₂SO₄·10H₂O (50 mg) filtered and concentrated under reduced pressure to give a residue. The residue was used for the next step without further purification. Intermediate 1-2 (120 mg, 245.10 μmol, 42.29% yield) was obtained as yellow oil. LCMS (Method D): Rt=0.468 min, M+H=490.1,

### Step 2: Synthesis of Intermediate 1-3

To a solution of Intermediate 1-2 (80 mg, 163.40 μmol, 1 eq) in DCM (1 mL) was added SOCl₂ (29.16 mg, 245.10 μmol, 17.80 μL, 1.5 eq) and DMAP (9.98 mg, 81.70 μmol, 0.5 eq). The mixture was stirred at 0° C. for 1 hr. The reaction mixture was poured into water (3 mL) and extracted with EA (2 mL*3). The combined organic layers were dried over Na₂SO₄, filtered and concentrated under reduced pressure to give a residue. The crude product was purified by reversed-phase HPLC (0.1% FA condition), and the eluent was lyophilized to give product. Intermediate 1-3 (20 mg, 39.37 μmol, 24.09% yield) was obtained as pink oil. LCMS (Method D): Rt=0.376 min, M+H=408.2.

### Step 3: Synthesis of Intermediate 1-5

To a solution of Intermediate 1-3 (20 mg, 39.37 μmol, 1 eq) and Intermediate 1-4 (23.21 mg, 39.37 μmol, 1 eq) in ACN (0.5 mL) was added DIEA (20.35 mg, 157.46 μmol, 27.43 μL, 4 eq) and KI (3.27 mg, 19.68 μmol, 0.5 eq). The mixture was stirred at 25° C. for 1 hr. The reaction mixture was diluted with water (10 mL) and extracted with EA (5 mL*3). The combined organic layers were dried over Na₂SO₄, filtered and concentrated under reduced pressure to give a residue. The residue was purified by prep-HPLC (0.1% FA condition), and the eluent was lyophilized to give product. Intermediate 1-5 (25 mg, 23.56 μmol, 59.84% yield) was obtained as yellow oil. LCMS (Method D): Rt=0.407 min, M+H=531.6.

### Step 4: Synthesis of I-436

To a solution of Intermediate 1-5 (20 mg, 18.84 μmol, 1 eq) in DCM (0.1 mL) was added HCl/dioxane (2 M, 0.1 mL, 10.61 eq). The mixture was stirred at 25° C. for 0.5 hr. The reaction mixture was concentrated under reduced pressure to give a residue. The residue was purified by prep-HPLC (0.1% HCl condition) and the eluent was lyophilized to give the product. I-436 (8 mg, 8.02 μmol, 42.55% yield, 100% purity, HCl salt) was obtained as a yellow solid. LCMS (Method D): Rt=0.303 min, M+H=961.5. ¹H NMR (400 MHz, METHANOL-d4) δ=8.85 (d, J=2.0 Hz, 1H), 8.38 (d, J=7.6 Hz, 1H), 8.12 (d, J=2.0 Hz, 1H), 8.02-7.93 (m, 1H), 7.92-7.81 (m, 2H), 7.54-7.48 (m, 3H), 7.47-7.40 (m, 2H), 7.38 (d, J=4.4 Hz, 3H), 7.35-7.27 (m, 2H), 7.17 (t, J=9.2 Hz, 1H), 4.62 (s, 2H), 4.50 (d, J=16.8 Hz, 2H), 4.39 (s, 2H), 4.33 (s, 2H), 4.20 (s, 2H), 3.87 (s, 6H), 3.82-3.71 (m, 5H), 3.70-3.51 (m, 5H), 3.47-3.34 (m, 6H), 2.78-2.72 (m, 2H), 2.40 (s, 3H), 2.30-2.21 (m, 2H), 1.94-1.57 (m, 2H), 1.38-1.26 (m, 5H).

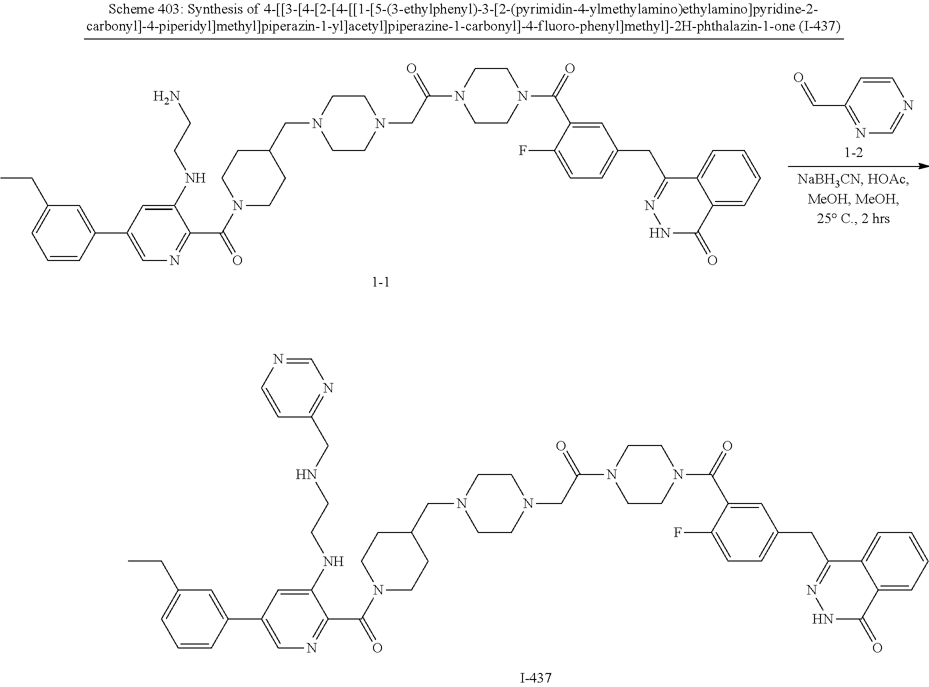 [SMILES: CCc1cccc(-c2cnc(C(=O)N3CCC(CN4CCN(CC(=O)N5CCN(C(=O)c6cc(Cc7n[nH]c(=O)c8ccccc78)ccc6F)CC5)CC4)CC3)c(NCCN)c2)c1 ; O=Cc1ccncn1 ; CCc1cccc(-c2cnc(C(=O)N3CCC(CN4CCN(CC(=O)N5CCN(C(=O)c6cc(Cc7n[nH]c(=O)c8ccccc78)ccc6F)CC5)CC4)CC3)c(NCCNCc3ccncn3)c2)c1]

To a solution of intermediate 1-1 (40 mg, 44.77 μmol, 1 eq, HCl salt) in MeOH (0.5 mL) was added TEA (18.12 mg, 179.07 μmol, 24.92 μL, 4 eq) dropwise at 25° C. and the mixture was stirred for 10 min. Then intermediate 1-2 (5.32 mg, 49.25 μmol, 1.1 eq) and HOAc (10.75 mg, 179.07 μmol, 10.25 μL, 4 eq) as added at 25° C. and the mixture was stirred for 20 min. NaBH₃CN (16.88 mg, 268.61 μmol, 6 eq) was added. The resulting mixture was stirred at 25° C. for 1 hr. The mixture was diluted with water (5 mL) and extracted with DCM (5 mL*3). The combined organic layer was dried over Na₂SO₄, filtered and concentrated in vacuo to give a residue. The residue was purified by prep-HPLC (column: CD04-Welch Utimate C18 150*25*7 um; mobile phase: [water (TFA)-ACN]; gradient: 13%-43% B over 10 min). Then the eluent was concentrated in vacuo and lyophilized. I-437 (12 mg, 11.07 μmol, 24.72% yield, 98.053% purity, TFA salt) was obtained as a yellow solid. LCMS (Method D): Retention time=0.321 min, (M+H)=949.4. ¹H NMR (400 MHz, METHANOL-d4) δ=9.16 (s, 1H), 8.81 (d, J=5.2 Hz, 1H), 8.37 (d, J=7.6 Hz, 1H), 8.16 (s, 1H), 7.95 (t, J=6.8 Hz, 1H), 7.89-7.81 (m, 2H), 7.57-7.55 (m, 1H), 7.53-7.48 (m, 3H), 7.45-7.38 (m, 2H), 7.37-7.28 (m, 2H), 7.20-7.14 (m, 1H), 4.79-4.69 (m, 2H), 4.54 (s, 2H), 4.39 (s, 2H), 3.82 (d, J=11.6 Hz, 4H), 3.77 (s, 4H), 3.71 (d, J=8.0 Hz, 2H), 3.60 (s, 2H), 3.54 (s, 2H), 3.44 (s, 4H), 3.17 (s, 6H), 2.95 (s, 2H), 2.77-2.71 (m, 2H), 2.20-2.14 (m, 1H), 2.01-1.93 (m, 1H), 1.85-1.78 (m, 1H), 1.41 (d, J=11.6 Hz, 2H), 1.32-1.26 (m, 3H).

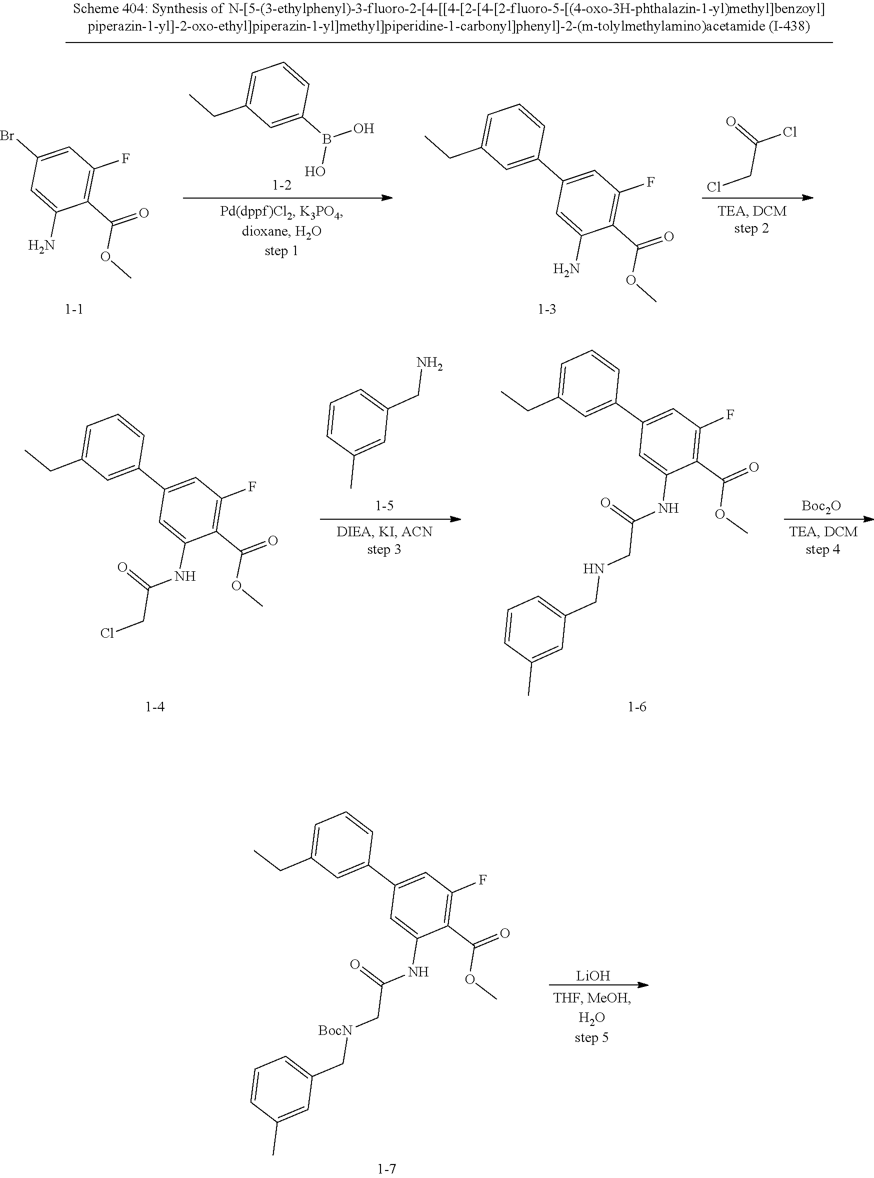

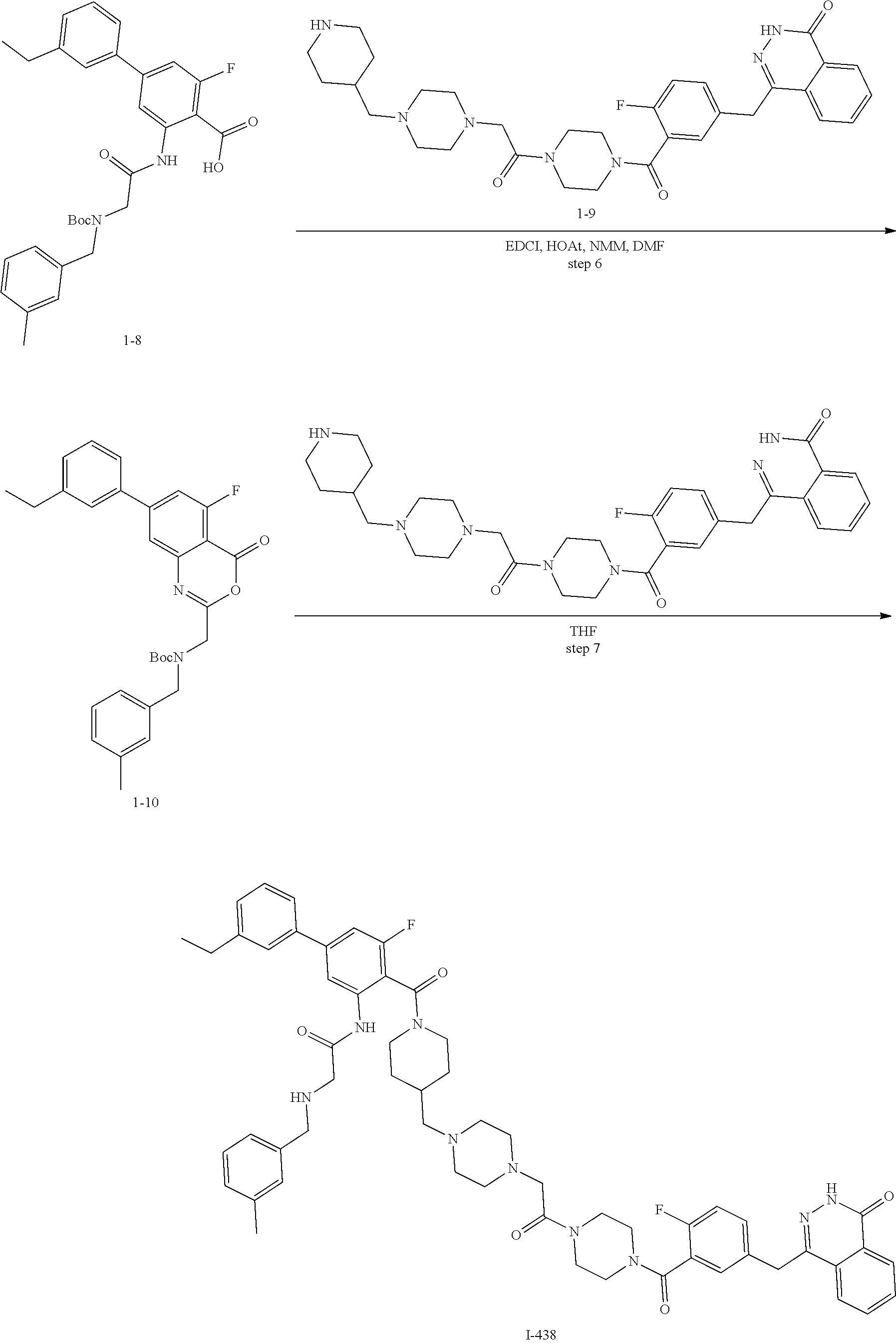

### Step 1: Synthesis of Intermediate 1-3

A mixture of Intermediate 1-1 (1 g, 4.03 mmol, 1 eq), Intermediate 1-2 (1.21 g, 8.06 mmol, 2 eq), Pd(dppf)Cl₂ (294.98 mg, 403.15 μmol, 0.1 eq), and K₃PO₄ (2.57 g, 12.09 mmol, 3 eq) in dioxane (7.5 mL) and H₂O (1.5 mL) was degassed and purged with N₂ 3 times, and then the mixture was stirred at 90° C. for 1 hr under N₂ atmosphere. The reaction mixture was diluted with water (10 mL) and extracted with EA (10 mL*3). The combined organic layers were dried over Na₂SO₄, filtered and concentrated under reduced pressure to give a residue. The residue was purified by flash silica gel chromatography (ISCO; 40 g SepaFlash Silica Flash Column, Eluent of 0˜100% Petroleum ether/Ethyl acetate @60 mL/min). The eluent was concentrated under reduced pressure to give a residue. Intermediate 1-3 (1.2 g, crude) was obtained as yellow oil. LCMS (Method D): Rt=0.500 min, M+H=274.1. ¹H NMR (400 MHz, METHANOL-d4) δ=7.42-7.37 (m, 2H), 7.35-7.30 (m, 1H), 7.22 (d, J=7.6 Hz, 1H), 6.82-6.78 (m, 1H), 6.56-6.53 (m, 1H), 3.87 (s, 3H), 2.72-2.67 (m, 2H), 1.26 (t, J=7.6 Hz, 3H).

### Step 2: Synthesis of Intermediate 1-4

To a solution of Intermediate 1-3 (800 mg, 2.93 mmol, 1 eq) in DCM (8 mL) was added TEA (888.59 mg, 8.78 mmol, 1.22 mL, 3 eq), 2-chloroacetyl chloride (396.72 mg, 3.51 mmol, 279.78 μL, 1.2 eq) at 0° C. The mixture was stirred at 0° C. for 3 hr. The reaction mixture was concentrated under reduced pressure to give a residue. The residue was used for the next step without further purification. Intermediate 1-4 (910 mg, 2.60 mmol, 88.88% yield) was obtained as a yellow oil. LCMS (Method D): Rt=0.512 min, M+Na=372.0.

### Step 3: Synthesis of Intermediate 1-6

To a solution of Intermediate 1-4 (900 mg, 2.57 mmol, 1 eq) and Intermediate 1-5 (311.80 mg, 2.57 mmol, 322.77 μL, 1 eq) in ACN (10 mL) was added DIEA (1.33 g, 10.29 mmol, 1.79 mL, 4 eq) and KI (213.56 mg, 1.29 mmol, 0.5 eq). The mixture was stirred at 40° C. for 12 hr. The reaction mixture was poured into water (10 mL) and extracted with DCM (5 mL*4). The combined organic layers were dried over Na₂SO₄, filtered and concentrated under reduced pressure to give a residue. The residue was purified by flash silica gel chromatography (ISCO; 40 g SepaFlash Silica Flash Column, Eluent of 0˜1000% Petroleum ether/Ethyl acetate@60 mL/min). The eluent was concentrated under reduced pressure to give a residue. Intermediate 1-6 (460 mg, 1.06 mmol, 41.15% yield) was obtained as a yellow oil. LCMS (Method D): Rt=0.388 min, M+H=435.1.

### Step 4: Synthesis of Intermediate 1-7

To a solution of Intermediate 1-6 (400 mg, 920.60 μmol, 1 eq) and Boc₂O (301.37 mg, 1.38 mmol, 317.24 μL, 1.5 eq) in DCM (4 mL) was added TEA (139.73 mg, 1.38 mmol, 192.20 μL, 1.5 eq). The mixture was stirred at 25° C. for 1 hr. The reaction mixture was poured into water (10 mL) and extracted with DCM (5 mL*4). The combined organic layers were dried over Na₂SO₄, filtered and concentrated under reduced pressure to give a residue. The residue was purified by flash silica gel chromatography (ISCO; 40 g SepaFlash Silica Flash Column, Eluent of 0˜100% Petroleum ether/Ethyl acetate@60 mL/min). The eluent was concentrated under reduced pressure to give a residue. Intermediate 1-7 (360 mg, 673.38 μmol, 73.15% yield) was obtained as a yellow oil. LCMS (Method D): Rt=0.610 min, M+H=435.2. ¹H NMR (400 MHz, METHANOL-d4) δ=8.44 (s, 1H), 7.46 (d, J=15.6 Hz, 2H), 7.37 (t, J=7.6 Hz, 1H), 7.27 (d, J=7.6 Hz, 1H), 7.24-7.16 (m, 2H), 7.14-7.02 (m, 3H), 4.57 (s, 2H), 4.07-3.91 (m, 5H), 2.74-2.68 (m, 2H), 2.27 (s, 3H), 1.57-1.43 (m, 9H), 1.27 (t, J=7.6 Hz, 3H).

### Step 5: Synthesis of Intermediate 1-8

To a solution of Intermediate 1-7 (350 mg, 654.67 μmol, 1 eq) in THF (1 mL), MeOH (1 mL), and H₂O (1 mL) was added LiOH·H₂O (54.94 mg, 1.31 mmol, 2 eq). The mixture was stirred at 25° C. for 1 hr. The mixture was adjusted to pH=5 with citric acid and extracted with EA (5 mL*3). The combined organic layers were dried over Na₂SO₄, filtered and concentrated under reduced pressure to give a residue. The residue was used for the next step without further purification. Intermediate 1-8 (300 mg, 576.27 μmol, 88.02% yield) was obtained as a yellow oil.

LCMS (Method D): Rt=0.560 min, M+H=521.8. ¹H NMR (400 MHz, METHANOL-d4) δ=8.60 (s, 1H), 7.53-7.45 (m, 2H), 7.39 (t, J=7.6 Hz, 1H), 7.28 (d, J=7.6 Hz, 1H), 7.25-7.18 (m, 2H), 7.16-7.03 (m, 3H), 4.58 (s, 2H), 4.05-3.91 (m, 2H), 2.76-2.70 (m, 2H), 2.29 (s, 3H), 1.54-1.44 (m, 9H), 1.28 (t, J=7.6 Hz, 3H).

### Step 6: Synthesis of Intermediate 1-10

To a solution of Intermediate 1-8 (50 mg, 96.04 μmol, 1 eq) in DMF (1 mL) was added HOAt (13.07 mg, 96.04 μmol, 13.44 μL, 1 eq) and EDCI (55.24 mg, 288.13 μmol, 3 eq), NMM (48.57 mg, 480.22 μmol, 52.80 μL, 5 eq), then Intermediate 1-9 (56.64 mg, 96.04 μmol, 1 eq) was added. The mixture was stirred at 60° C. for 1 hr. The reaction mixture was poured into water (5 mL) and extracted with DCM 8 mL (2 mL*4). The combined organic layers were dried over Na₂SO₄, filtered and concentrated under reduced pressure to give a residue. The residue was used for the next step without further purification. Intermediate 1-10 (60 mg, crude) was obtained as a yellow oil. LCMS (Method D): Rt=0.678 min, M+H=503.2

### Step 7: Synthesis of I-438

A solution of Intermediate 1-9 (30 mg, 50.87 μmol, 1 eq) and Intermediate 1-10 (30.68 mg, 61.05 μmol, 1.2 eq) in THF (1 mL) was stirred at 80° C. for 12 hr. The reaction mixture was concentrated under reduced pressure to give a residue. The residue was purified by prep-HPLC (column: Welch Xtimate C18 150*30 mm*5 um; mobile phase: [water(FA)-ACN]; gradient: 17%-47% B over 10 min) and the eluent was lyophilized to give product. I-438 (17 mg, 16.13 μmol, 31.71% yield, 98.511% purity, FA salt) was obtained as a white solid. LCMS (Method D): Rt=0.414 min, M+H=992.5. ¹H NMR (400 MHz, METHANOL-d4) δ=8.46 (d, J=2.4 Hz, 1H), 8.37 (d, J=7.6 Hz, 1H), 8.18-8.04 (m, 1H), 7.98-7.91 (m, 1H), 7.91-7.79 (m, 2H), 7.53-7.42 (m, 3H), 7.42-7.34 (m, 2H), 7.34-7.19 (m, 5H), 7.19-7.13 (m, 1H), 7.10 (d, J=6.4 Hz, 1H), 4.72-4.60 (m, 1H), 4.38 (s, 2H), 3.87-3.77 (m, 3H), 3.75-3.59 (m, 4H), 3.52 (d, J=2.8 Hz, 2H), 3.45 (s, 2H), 3.35 (s, 2H), 3.29-3.18 (m, 2H), 3.16-3.03 (m, 1H), 2.89 (t, J=13.2 Hz, 1H), 2.78-2.67 (m, 3H), 2.65-2.46 (m, 7H), 2.40-2.29 (m, 4H), 2.23-2.18 (m, 1H), 1.93-1.69 (m, 3H), 1.28 (t, J=7.6 Hz, 3H), 1.26-0.96 (m, 2H).

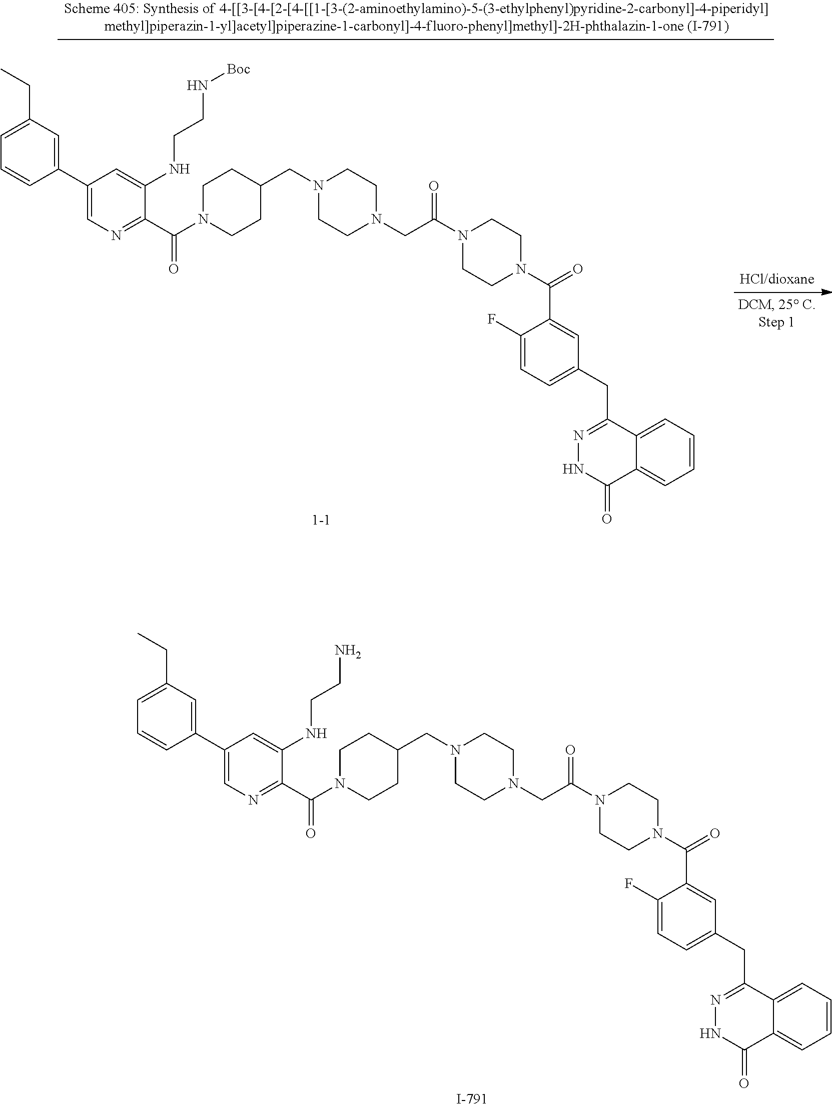 [SMILES: CCc1cccc(-c2cnc(C(=O)N3CCC(CN4CCN(CC(=O)N5CCN(C(=O)c6cc(Cc7n[nH]c(=O)c8ccccc78)ccc6F)CC5)CC4)CC3)c(NCCNC(=O)OC(C)(C)C)c2)c1 ; CCc1cccc(-c2cnc(C(=O)N3CCC(CN4CCN(CC(=O)N5CCN(C(=O)c6cc(Cc7n[nH]c(=O)c8ccccc78)ccc6F)CC5)CC4)CC3)c(NCCN)c2)c1]

To a solution of intermediate 1-1 (20 mg, 20.90 μmol, 1 eq) in DCM (0.5 mL) was added HCl/dioxane (2 M, 0.2 mL, 19.14 eq). The mixture was stirred at 25° C. for 0.5 hr. The reaction mixture was concentrated in vacuo to give a residue. The residue was diluted with water and MeCN and lyophilized directly without further purification. I-791 (9 mg, 10.05 μmol, 48.09% yield, 99.767% purity, HCl salt) was obtained as a yellow solid. LCMS (Method D): Retention time=0.334 min, (M+H)=857.6. ¹H NMR (400 MHz, METHANOL-d4) δ=8.41-8.31 (m, 2H), 8.01-7.92 (m, 2H), 7.91-7.80 (m, 2H), 7.67-7.60 (m, 2H), 7.54-7.46 (m, 2H), 7.43-7.33 (m, 2H), 7.17 (t, J=8.8 Hz, 1H), 4.53 (d, J=16.4 Hz, 2H), 4.39 (s, 2H), 3.88 (s, 7H), 3.77 (t, J=6.4 Hz, 6H), 3.63-3.50 (m, 3H), 3.41 (s, 2H), 3.33 (s, 4H), 3.26 (s, 2H), 3.25-3.20 (m, 2H), 2.80-2.74 (m, 2H), 2.40-2.29 (m, 1H), 2.06 (d, J=12.0 Hz, 2H), 1.59-1.43 (m, 2H), 1.30 (t, J=7.6 Hz, 3H).

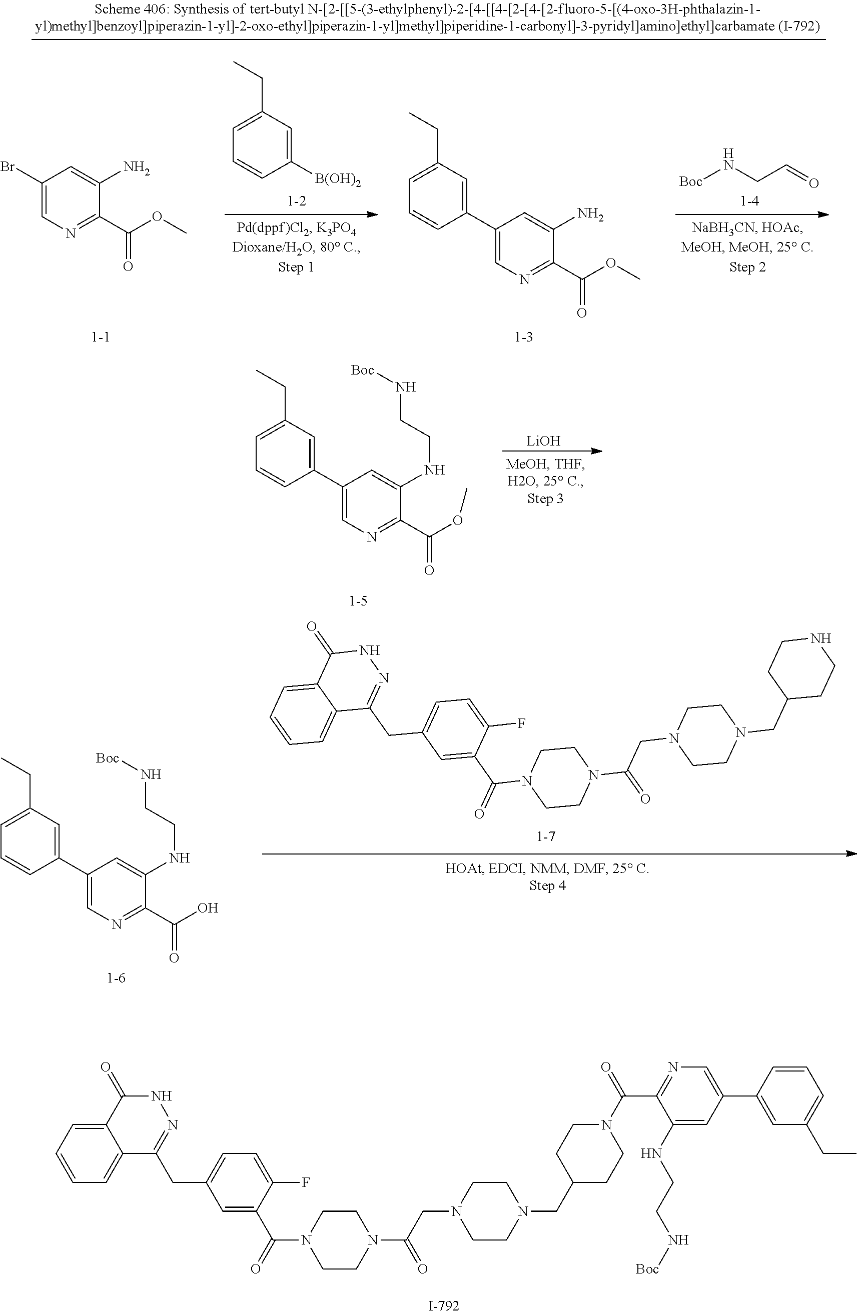

To a solution of intermediate 1-1 (2 g, 8.66 mmol, 1 eq) and intermediate 1-2 (1.56 g, 10.39 mmol, 1.2 eq) in dioxane (15 mL) and H₂O (3 mL) was added Pd(dppf)Cl₂ (633.38 mg, 865.63 μmol, 0.1 eq) and K₃PO₄ (5.51 g, 25.97 mmol, 3 eq). The mixture was stirred at 80° C. for 1 hr. The mixture was diluted with water (15 mL) and extracted with DCM (10 mL*3). The combined organic layer was dried over Na₂SO₄, filtered and concentrated in vacuo to give a residue. The residue was purified by column chromatography (SiO₂, PE:EA=10:1 to PE:EA=0:1), then the organic liquid was concentrated in vacuo to give a residue. Intermediate 1-3 (2 g, 7.73 mmol, 89.25% yield, 99% purity) was obtained as a brown solid. LCMS (Method D): Retention time=0.337 min, (M+H)=257.1.

### Step 2: Synthesis of Intermediate 1-5

To a solution of intermediate 1-3 (200 mg, 780.34 μmol, 1 eq) and intermediate 1-4 (149.06 mg, 936.41 μmol, 1.2 eq) in MeOH (2 mL) was added CH₃COOH (281.16 mg, 4.68 mmol, 268.03 μL, 6 eq). The mixture was stirred at 25° C. for 0.5 h, then NaBH₃CN (196.15 mg, 3.12 mmol, 4 eq) was added and the mixture was stirred at 25° C. for 1 h. The mixture was diluted with water (8 mL) and extracted with DCM (8 mL*3). The combined organic layer was dried over Na₂SO₄, filtered and concentrated in vacuo to give a residue. The residue was purified by column chromatography (SiO₂, PE:EA=10:1 to DCM:MeOH=0:1), then the organic liquid was concentrated in vacuo to give a residue. Intermediate 1-5 (100 mg, 250.32 μmol, 32.08% yield) was obtained as a yellow solid. LCMS (Method D): Retention time=0.439 min, (M+H)=400.5.

### Step 3: Synthesis of Intermediate 1-6

To a solution of intermediate 1-5 (100 mg, 250.32 μmol, 1 eq) in THF (0.3 mL), H₂O (0.3 mL), and MeOH (0.3 mL) was added LiOH·H₂O (21.01 mg, 500.65 μmol, 2 eq). The mixture was stirred at 25° C. for 0.5 hr. The reaction mixture was concentrated in vacuo to remove THF and MeOH to give a residue, the residue was diluted with water (5 mL) and extracted with DCM (5 mL*3), then the aqueous phase was acidified with hydrochloric acid (1 M) to pH=5-6, and the mixture was extracted with DCM (5 mL*3). The combined organic layer was dried over Na₂SO₄, filtered and concentrated in vacuo to give a residue. The residue was used in the next step directly without further purification. Intermediate 1-6 (95 mg, 246.46 μmol, 98.46% yield) was obtained as a yellow solid. LCMS (Method D): Retention time=0.415 min, (M+H)=386.1.

### Step 4: Synthesis of I-792

To a solution of intermediate 1-6 (45 mg, 116.74 μmol, 1 eq) and intermediate 1-7 (87.72 mg, 140.09 μmol, 1.2 eq, HCl salt) in DMF (1 mL) was added EDCI (67.14 mg, 350.23 μmol, 3 eq) and HOAt (15.89 mg, 116.74 μmol, 16.33 μL, 1 eq), and NMM (59.04 mg, 583.72 μmol, 64.18 μL, 5 eq). The mixture was stirred at 25° C. for 0.5 hr. The mixture was diluted with water (5 mL) and extracted with DCM (5 mL*3). The combined organic layer was dried over Na₂SO₄, filtered and concentrated in vacuo to give a residue. The mixture was purified by prep-HPLC (column: Phenomenex luna C18 150*25 mm*10 um; mobile phase: [water (FA)-ACN]; gradient: 23%-53% B over 10 min). Then the eluent was concentrated in vacuo and lyophilized. I-792 (19 mg, 19.85 μmol, 17.00% yield, 100% purity) was obtained as a white solid. LCMS (Method D): Retention time=0.396 min, (M+H)=957.8. ¹H NMR (400 MHz, METHANOL-d4) δ=8.37 (d, J=7.6 Hz, 1H), 8.04 (d, J=1.6 Hz, 1H), 7.98-7.92 (m, 1H), 7.91-7.80 (m, 2H), 7.54-7.46 (m, 3H), 7.44 (s, 1H), 7.41-7.34 (m, 2H), 7.27 (d, J=7.6 Hz, 1H), 7.16 (t, J=9.6 Hz, 1H), 4.82-4.80 (m, 2H), 4.71-4.60 (m, 1H), 4.38 (s, 2H), 3.82-3.77 (m, 1H), 3.77-3.63 (m, 4H), 3.56-3.49 (m, 2H), 3.38-3.35 (m, 2H), 3.34 (s, 4H), 3.30-3.29 (m, 2H), 3.27 (s, 2H), 3.18-3.09 (m, 1H), 2.96-2.85 (m, 1H), 2.74-2.70 (m, 2H), 2.62 (s, 4H), 2.59 (d, J=2.4 Hz, 3H), 2.40-2.30 (m, 2H), 1.97-1.86 (m, 2H), 1.81-1.68 (m, 1H), 1.40 (s, 9H), 1.29 (t, J=7.6 Hz, 3H).

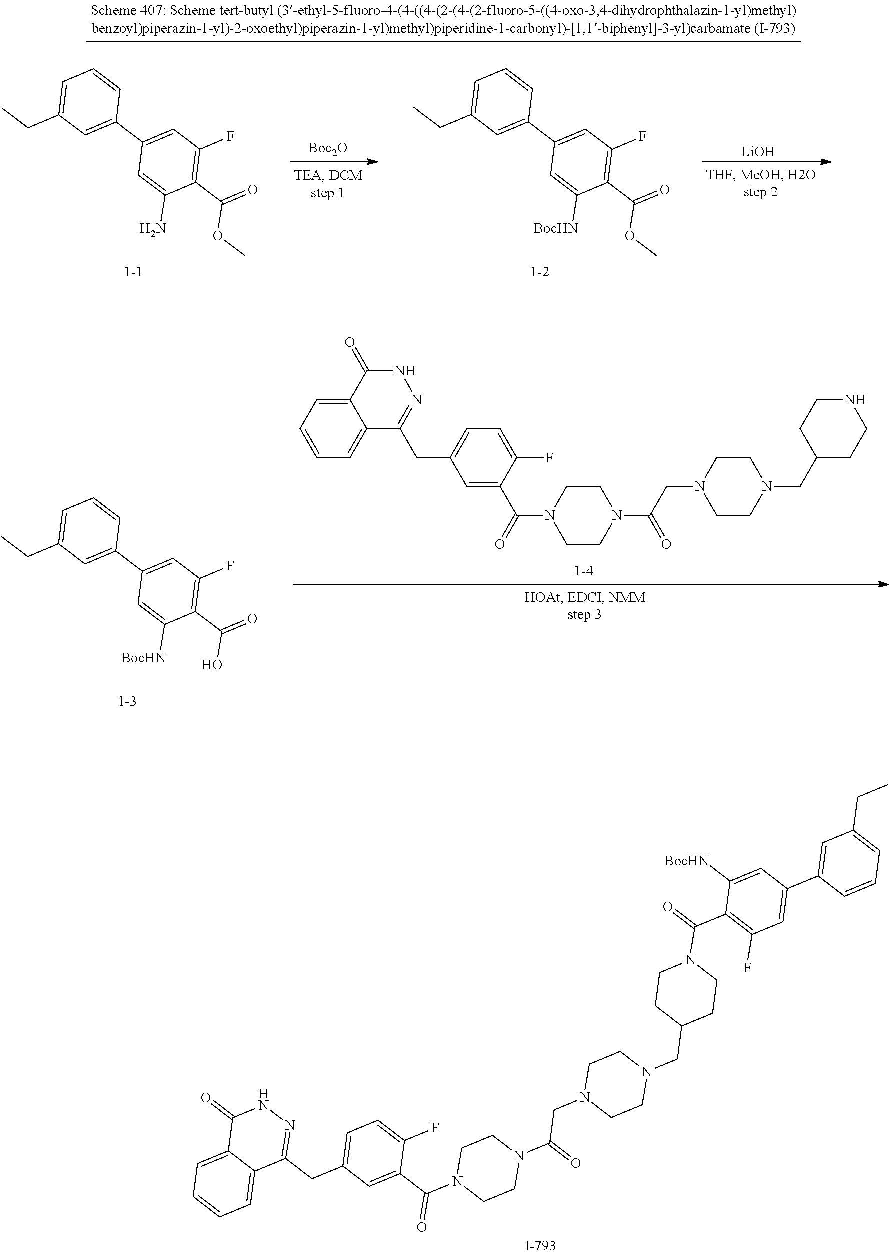

### Step 1: Synthesis of Intermediate 1-2

To a solution of Intermediate 1-1 (180 mg, 658.61 μmol, 1 eq) in DCM (1.8 mL) was added Boc₂O (172.49 mg, 790.34 μmol, 181.57 μL, 1.2 eq) and TEA (133.29 mg, 1.32 mmol, 183.34 μL, 2 eq) and DMAP (8.05 mg, 65.86 μmol, 0.1 eq). The mixture was stirred at 25° C. for 1 hr. The reaction mixture was concentrated under reduced pressure to give a residue. The residue was purified by flash silica gel chromatography (ISCO; 20 g SepaFlash Silica Flash Column, Eluent of 0˜100% Ethyl acetate/Petroleum ethergradient @40 mL/min) and the eluent was concentrated to give product. Intermediate 1-2 (80 mg, 214.24 μmol, 32.53% yield) was obtained as a white solid. LCMS (Method D): Retention time: 0.534 min, (M+Na)=396.1. ¹H NMR (400 MHz, CHLOROFORM-d) δ=9.93 (s, 1H), 8.49 (s, 1H), 7.51-7.43 (m, 2H), 7.37 (t, J=7.2 Hz, 1H), 7.26-7.23 (m, 1H), 7.01-6.98 (m, 1H), 3.98 (s, 3H), 2.76-2.70 (m, 2H), 1.57-1.54 (m, 9H), 1.29 (t, J=7.6 Hz, 3H).

### Step 2: Synthesis of Intermediate 1-3

To a solution of Intermediate 1-2 (40 mg, 107.12 μmol, 1 eq) in THF (0.1 mL), MeOH (0.1 mL) and H₂O (0.1 mL) was added LiOH·H₂O (8.99 mg, 214.24 μmol, 2 eq). The mixture was stirred at 25° C. for 1 hr. To the reaction mixture was added H₂O (2 mL) and then it was extracted with DCM (2 mL*3), the combined organic phase was dried by Na₂SO₄, filtered and concentrated. The crude was used for the next step. Intermediate 1-3 (30 mg, 78.47 μmol, 73.25% yield, 94% purity) was obtained as a white solid. LCMS (Method D): Retention time: 0.480 min, (M+Na)=396.1.

### Step 3: Synthesis of I-793

To a solution of Intermediate 1-3 (11.48 mg, 31.94 μmol, 1 eq) and Intermediate 1-4 (20 mg, 31.94 μmol, 1 eq, HCl salt) in DMF (0.2 mL) was added EDCI (12.25 mg, 63.88 μmol, 2 eq) and HOAt (4.35 mg, 31.94 μmol, 4.47 μL, 1 eq) and NMM (16.15 mg, 159.70 μmol, 17.56 μL, 5 eq). The mixture was stirred at 25° C. for 1 hr. The reaction mixture was diluted with MeOH (1 ml). The residue was purified by prep-HPLC (column: Phenomenex luna C18 150*25 mm*10 um; mobile phase: [water (FA)-ACN]; gradient: 30%-60% B over 8 min) and the eluent was concentrated to remove ACN and lyophilized to give the product. I-793 (7.5 mg, 7.58 μmol, 23.74% yield, 98.786% purity, FA salt) was obtained as a white solid. LCMS (Method D): Retention time: 0.414 min, (M+H)=931.4. ¹H NMR (400 MHz, METHANOL-d4) δ=8.54-8.45 (m, 1H), 8.45-8.32 (m, 1H), 8.00-7.94 (m, 1H), 7.93-7.82 (m, 2H), 7.77 (d, J=6.4 Hz, 1H), 7.55-7.43 (m, 3H), 7.42-7.35 (m, 2H), 7.32-7.12 (m, 3H), 4.73-4.64 (m, 1H), 4.40 (s, 2H), 3.85-3.62 (m, 5H), 3.56-3.53 (m, 2H), 3.35 (s, 2H), 3.31-3.08 (m, 3H), 2.95-2.82 (m, 1H), 2.77-2.71 (m, 2H), 2.70-2.47 (m, 7H), 2.40-2.27 (m, 2H), 1.99-1.74 (m, 3H), 1.60-1.49 (m, 9H), 1.41-1.19 (m, 6H).

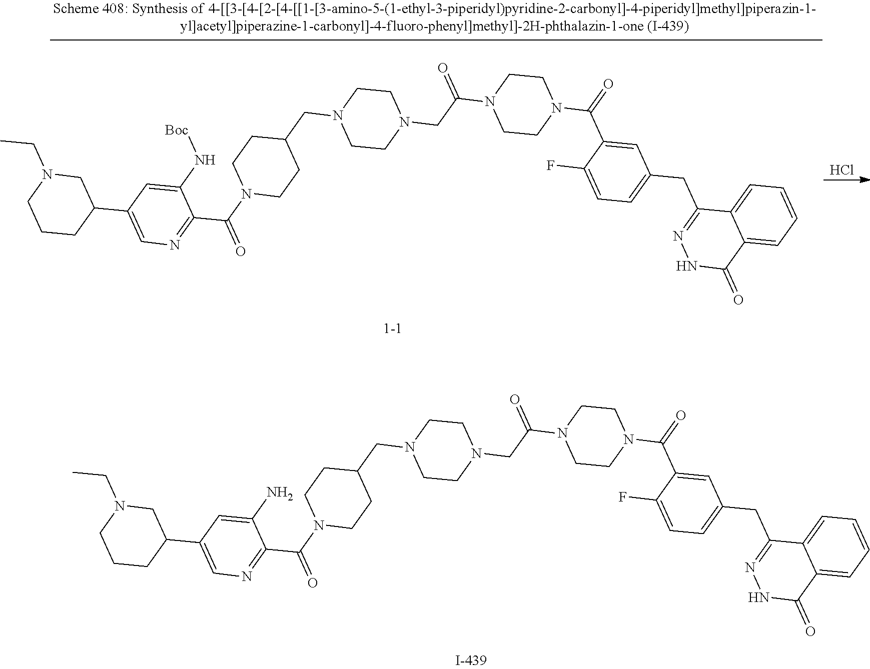 [SMILES: CCN1CCCC(c2cnc(C(=O)N3CCC(CN4CCN(CC(=O)N5CCN(C(=O)c6cc(Cc7n[nH]c(=O)c8ccccc78)ccc6F)CC5)CC4)CC3)c(NC(=O)OC(C)(C)C)c2)C1 ; Cl ; CCN1CCCC(c2cnc(C(=O)N3CCC(CN4CCN(CC(=O)N5CCN(C(=O)c6cc(Cc7n[nH]c(=O)c8ccccc78)ccc6F)CC5)CC4)CC3)c(N)c2)C1]

To a solution of intermediate 1-1 (40 mg, 43.43 μmol, 1 eq) in DCM (1 mL) was added HCl/dioxane (2 M, 108.56 μL, 5 eq), then the mixture was stirred at 20° C. for 1 hour. The reaction mixture was concentrated under reduced pressure to give a residue. The crude product was purified by reversed-phase HPLC (0.1% FA condition). The eluent was concentrated under reduced pressure to remove ACN and then lyophilized to give the product. I-439 (8.49 mg, 9.68 μmol, 22.30% yield, 98.883% purity, FA salt) was obtained as a white solid. LCMS (Method D): Retention time: 0.250 min, (M+H)=821.5. ¹H NMR (400 MHz, METHANOL-d4) δ=8.48 (s, 1H), 8.37 (d, J=7.6 Hz, 1H), 7.99-7.92 (m, 1H), 7.91-7.77 (m, 3H), 7.53-7.45 (m, 1H), 7.40-7.33 (m, 1H), 7.21-7.11 (m, 2H), 4.64 (d, J=11.6 Hz, 1H), 4.39 (s, 2H), 3.84-3.78 (m, 1H), 3.75 (s, 1H), 3.68 (s, 3H), 3.61-3.48 (m, 4H), 3.44 (s, 1H), 3.39-3.33 (m, 2H), 3.26 (s, 1H), 3.18-3.16 (m, 2H), 3.13-2.97 (m, 3H), 2.96-2.87 (m, 2H), 2.85-2.64 (m, 8H), 2.56-2.46 (m, 2H), 2.12-1.89 (m, 5H), 1.82-1.69 (m, 2H), 1.40-1.15 (m, 5H). ¹⁹F NMR (400 MHz, METHANOL-d4) δ=−120.672, −120.770.

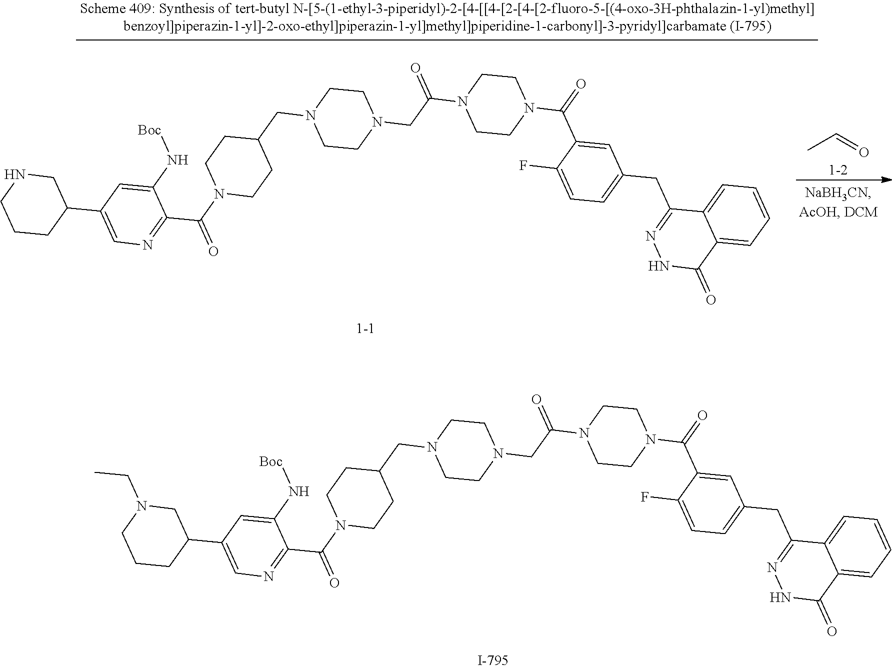

To a solution of intermediate 1-1 (90 mg, 100.78 μmol, 1 eq) and intermediate 1-2 (7.61 mg, 120.93 μmol, 9.69 μL, 1.2 eq) in DCM (1 mL) was added AcOH (12.10 mg, 201.55 μmol, 11.54 μL, 2 eq) and NaBH(OAc)₃ (106.79 mg, 503.89 μmol, 5 eq), then the mixture was stirred at 20° C. for 5 min. To the reaction mixture was added NaHCO₃ (about 10 mL) to adjust the pH to 7-8, and it was extracted with EA (10 mL*3). The combined organic layers were concentrated under reduced pressure to give a residue. The crude product was purified by reversed-phase HPLC (0.1% FA condition). The eluent was concentrated under reduced pressure to remove ACN and then lyophilized to give the product. I-795 (70 mg, 76.00 μmol, 75.41% yield, 100% purity, FA salt) was obtained as a white solid. LCMS (Method D): Retention time: 0.296 min, (M+H)=921.5. ¹H NMR (400 MHz, METHANOL-d4) δ=8.43 (s, 1H), 8.39-8.34 (m, 1H), 8.28-8.16 (m, 2H), 8.02-7.91 (m, 1H), 7.91-7.74 (m, 2H), 7.55-7.44 (m, 1H), 7.41-7.31 (m, 1H), 7.19-7.14 (m, 1H), 4.63 (d, J=12.4 Hz, 1H), 4.39 (s, 2H), 3.79 (d, J=4.4 Hz, 1H), 3.75-3.71 (m, 1H), 3.68 (s, 2H), 3.59 (d, J=12.0 Hz, 2H), 3.54-3.48 (m, 2H), 3.44 (s, 1H), 3.39-3.31 (m, 4H), 3.21-3.02 (m, 5H), 2.98-2.86 (m, 2H), 2.86-2.65 (m, 8H), 2.56-2.47 (m, 2H), 2.14-2.03 (m, 2H), 2.01-1.89 (m, 3H), 1.88-1.78 (m, 1H), 1.75-1.66 (m, 1H), 1.52 (s, 9H), 1.40-1.23 (m, 5H). ¹⁹F NMR (400 MHz, METHANOL-d4) δ=−120.792.

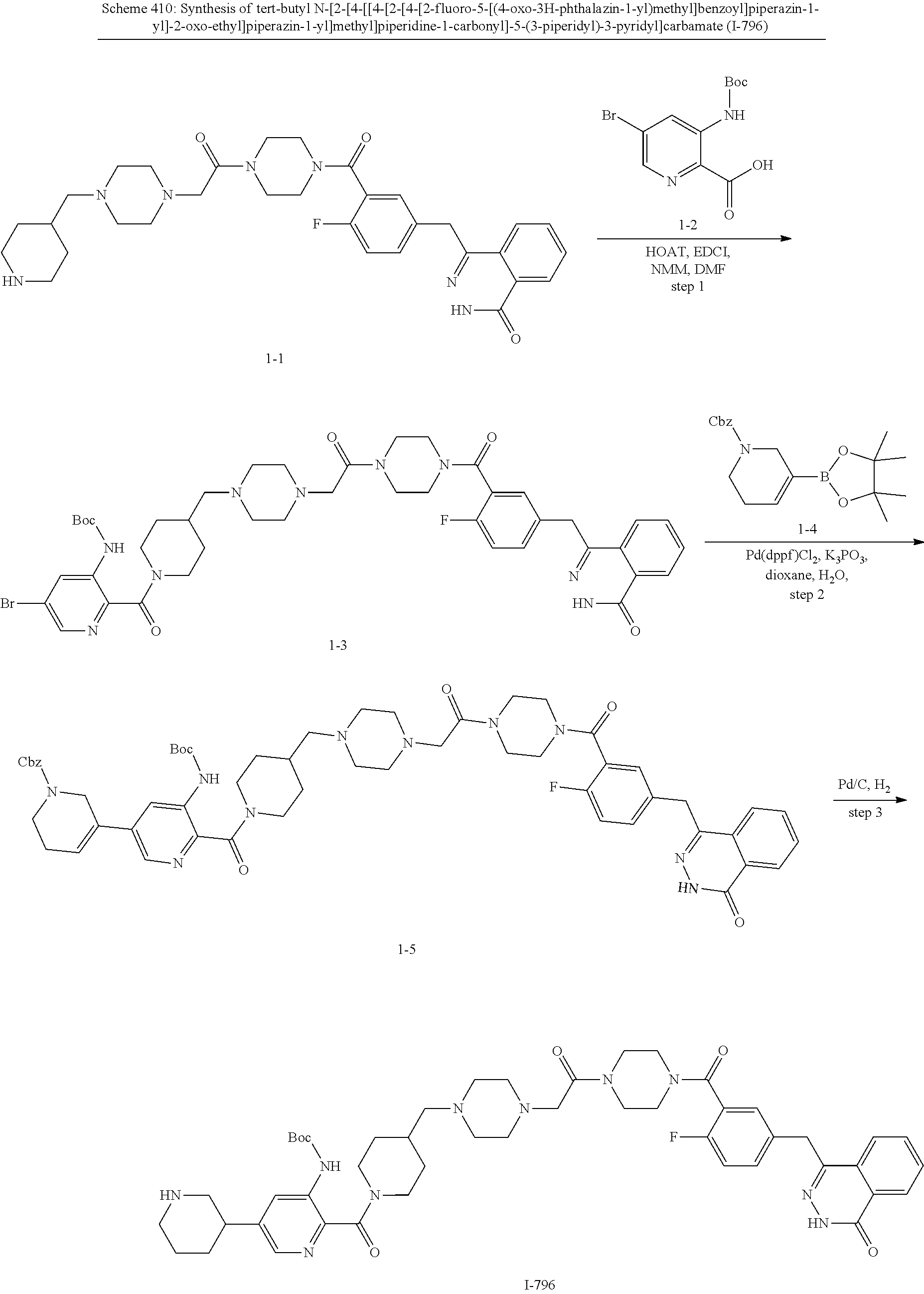

### Step 1: Synthesis of Intermediate 1-3

To a solution of intermediate 1-1 (250 mg, 423.94 μmol, 1 eq) and intermediate 1-2 (147.89 mg, 466.34 μmol, 1.1 eq) in DCM (2 mL) was added EDCI (243.81 mg, 1.27 mmol, 3 eq), HOAt (57.70 mg, 423.94 μmol, 59.30 μL, 1 eq) and NMM (214.40 mg, 2.12 mmol, 233.05 μL, 5 eq), then the mixture was stirred at 40° C. for 1 hour. The reaction mixture was diluted with water (80 mL) and extracted with DCM (30 mL*2). The combined organic layers were concentrated under reduced pressure to give a residue. Intermediate 1-3 (280 mg, 315.02 μmol, 74.31% yield) was obtained as a green solid. LCMS (Method D): Retention time: 0.364 min, (M+H)=888.3.

### Step 2: Synthesis of Intermediate 1-5

To a solution of intermediate 1-3 (280 mg, 315.02 μmol, 1 eq) and intermediate 1-4 (216.25 mg, 630.05 μmol, 2 eq) in H₂O (0.6 mL) and dioxane (3 mL) was added ditert-butyl (cyclopentyl)phosphane;dichloropalladium; iron (20.53 mg, 31.50 μmol, 0.1 eq) and K₃PO₄ (200.61 mg, 945.07 μmol, 3 eq). The mixture was stirred at 80° C. for 1 hour. The reaction mixture was diluted with water (80 mL) and extracted with EA (30 mL*2). The combined organic layers were concentrated under reduced pressure to give a residue. The crude product was purified by reversed-phase HPLC (0.1% NH₃·H₂O condition). The eluent was concentrated under reduced pressure to remove ACN and then lyophilized to give the product. Intermediate 1-5 (240 mg, 213.04 μmol, 67.63% yield, 91% purity) was obtained as a red solid. LCMS (Method D): Retention time: 0.418 min, (M+H)=1025.4.

### Step 3: Synthesis of I-796

To a solution of Pd(OH)₂ (164.38 mg, 234.11 μmol, 20% purity) in MeOH (3 mL) was added intermediate 1-5 (240 mg, 234.11 μmol, 1 eq), and the mixture was degassed and purged with H₂ 3 times, and then the mixture was stirred at 40° C. for 2 hrs under H₂ (15 psi) atmosphere. The reaction mixture was filtered and concentrated under reduced pressure to give a residue. The crude product was purified by reversed-phase HPLC (0.1% NH₃·H₂O condition). The eluent was concentrated under reduced pressure to remove ACN and then lyophilized to give the product. I-796 (155 mg, 173.56 μmol, 74.14% yield, 100% purity) was obtained as a white solid. LCMS (Method D): Retention time: 0.287 min, (M+H)=893.5. ¹H NMR (400 MHz, METHANOL-d4) δ=8.40-8.34 (m, 1H), 8.18 (d, J=1.6 Hz, 1H), 8.10 (s, 1H), 7.98-7.92 (m, 1H), 7.90-7.81 (m, 2H), 7.48 (s, 1H), 7.40-7.35 (m, 1H), 7.19-7.16 (m, 1H), 4.61 (d, J=12.8 Hz, 1H), 4.39 (s, 2H), 3.80-3.65 (m, 5H), 3.57-3.49 (m, 2H), 3.35 (s, 1H), 3.26 (s, 2H), 3.20-3.18 (m, 1H), 3.17-3.03 (m, 3H), 2.89-2.82 (m, 2H), 2.78-2.67 (m, 2H), 2.51 (s, 8H), 2.25-2.20 (m, 2H), 2.03 (d, J=8.2 Hz, 1H), 1.95-1.84 (m, 3H), 1.78-1.68 (m, 3H), 1.51 (s, 9H), 1.33-1.23 (m, 2H). ¹⁹F NMR (400 MHz, METHANOL-d4) δ=−120.725.

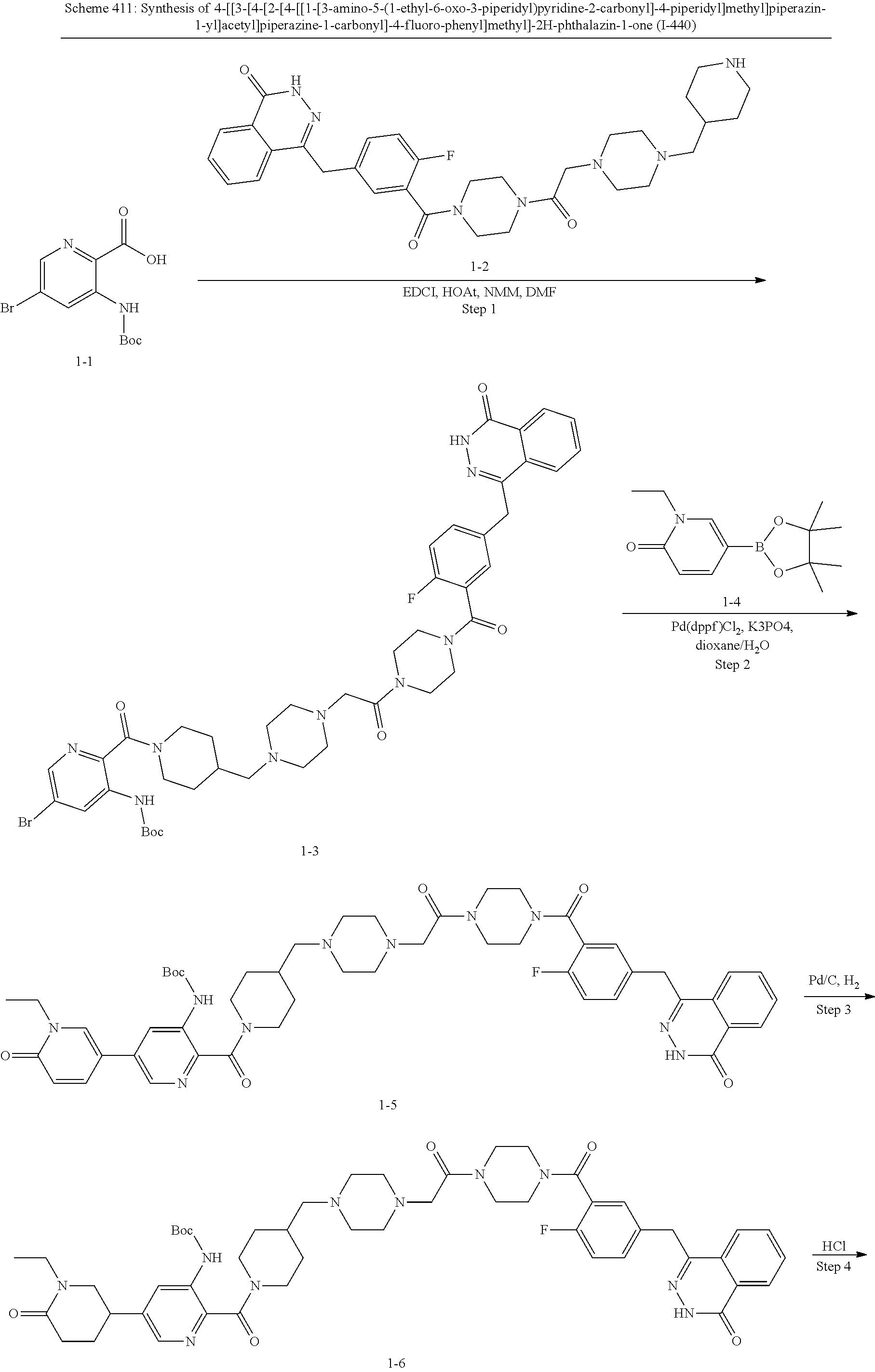

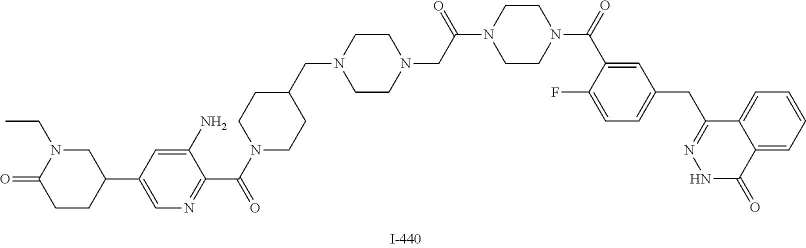 [SMILES: CCN1CC(c2cnc(C(=O)N3CCC(CN4CCN(CC(=O)N5CCN(C(=O)c6cc(Cc7n[nH]c(=O)c8ccccc78)ccc6F)CC5)CC4)CC3)c(N)c2)CCC1=O]

### Step 1: Synthesis of Intermediate 1-3.

To a mixture of Intermediate 1-1 (300 mg, 945.97 μmol, 1 eq) and Intermediate 1-2 (557.84 mg, 945.97 μmol, 1 eq) in DMF (5 mL) was added HOAt (128.76 mg, 945.97 μmol, 132.33 μL, 1 eq), EDCI (544.03 mg, 2.84 mmol, 3 eq) and NMM (478.41 mg, 4.73 mmol, 520.01 μL, 5 eq), then the mixture was stirred at 20° C. for 1 hour. The reaction mixture was diluted with water (100 mL) and extracted with EA (20 mL*3). The combined organic layers were concentrated under reduced pressure to give Intermediate 1-3 (300 mg, 337.53 μmol, 35.68% yield) as a white solid. LCMS (Method D): Retention time: 0.413 min, (M+H)=888.3.

### Step 2: Synthesis of Intermediate 1-5.

To a mixture of Intermediate 1-3 (300 mg, 337.53 μmol, 1 eq) and Intermediate 1-4 (168.16 mg, 675.05 μmol, 2 eq) in dioxane (5 mL) and H₂O (1 mL) was added Pd(dppf)Cl₂ (24.70 mg, 33.75 μmol, 0.1 eq), and K₃PO₄ (214.94 mg, 1.01 mmol, 3 eq), and then the mixture was stirred at 80° C. for 1 hour under N₂. The reaction mixture was concentrated under reduced pressure to give a residue. The residue was purified by flash silica gel chromatography (ISCO; 20 g SepaFlash Silica Flash Column, Eluent of 0˜50% Ethyl acetate/Petroleum ether gradient @50 mL/min). The eluent was concentrated under reduced pressure to give Intermediate 1-5 (300 mg, 322.21 μmol, 95.46% yield) as a white solid. LCMS (Method D): Retention time: 0.349 min, (M+H)=931.4.

### Step 3: Synthesis of Intermediate 1-6.

To a mixture of Intermediate 1-5 (100 mg, 107.40 μmol, 1 eq) in DMF (3 mL) was added Pd/C (11.43 mg, 10.74 μmol, 10% purity, 0.1 eq). The suspension was degassed and purged with N₂ 3 times, and then the mixture was degassed and purged with H₂ 3 times, then the mixture was stirred at 20° C. for 1 hour under H₂ atmosphere (15 psi). The reaction mixture was filtered and concentrated under reduced pressure to give a residue. The crude product was purified by prep-HPLC (column: Phenomenex luna C18 150*25 mm*10 um; mobile phase: [water (FA)-ACN]; gradient: 10%-40% B over 10 min). The eluent was concentrated under reduced pressure to remove MeCN and then lyophilized to give Intermediate 1-6 (80 mg, 85.55 μmol, 79.66% yield) as a white solid. LCMS (Method D): Retention time: 0.367 min, (M+H)=935.5.

### Step 4: Synthesis of I-440

To a mixture of Intermediate 1-6 (40 mg, 42.78 μmol, 1 eq) in DCM (1 mL) was added HCl/dioxane (2 M, 1 mL), then the mixture was stirred at 20° C. for 0.5 hour. The reaction mixture was concentrated under reduced pressure to give a residue. The crude product was purified by prep-HPLC (column: CD04-Welch Utimate C18 150*25*7 um; mobile phase: [water (FA)-ACN]; gradient: 1%-30% B over 8 min). The eluent was concentrated under reduced pressure to remove MeCN and then lyophilized to give I-440 (13.97 mg, 15.82 μmol, 36.98% yield, 99.77% purity, FA salt) as a white solid. LCMS (Method D): Retention time: 0.286 min, (M+H)=835.4. SFC: Rt=6.403 min, Rt=7.625 min. ¹H NMR (400 MHz, METHANOL-d₄) δ=8.41 (s, 1H), 8.37 (d, J=7.6 Hz, 1H), 7.99-7.91 (m, 1H), 7.90-7.77 (m, 3H), 7.50 (s, 1H), 7.37-7.35 (m, 1H), 7.20-7.12 (m, 2H), 4.65 (d, J=12.8 Hz, 1H), 4.38 (s, 2H), 3.81-3.64 (m, 5H), 3.55-3.41 (m, 8H), 3.35 (s, 2H), 3.18-3.08 (m, 2H), 2.97-2.70 (m, 9H), 2.60 (s, 2H), 2.52-2.46 (m, 2H), 2.07-1.87 (m, 4H), 1.72 (s, 1H), 1.29 (s, 2H), 1.17-1.13 (m, 3H). ¹⁹F NMR (376 MHz, CHLOROFORM-d) δ=−120.70.

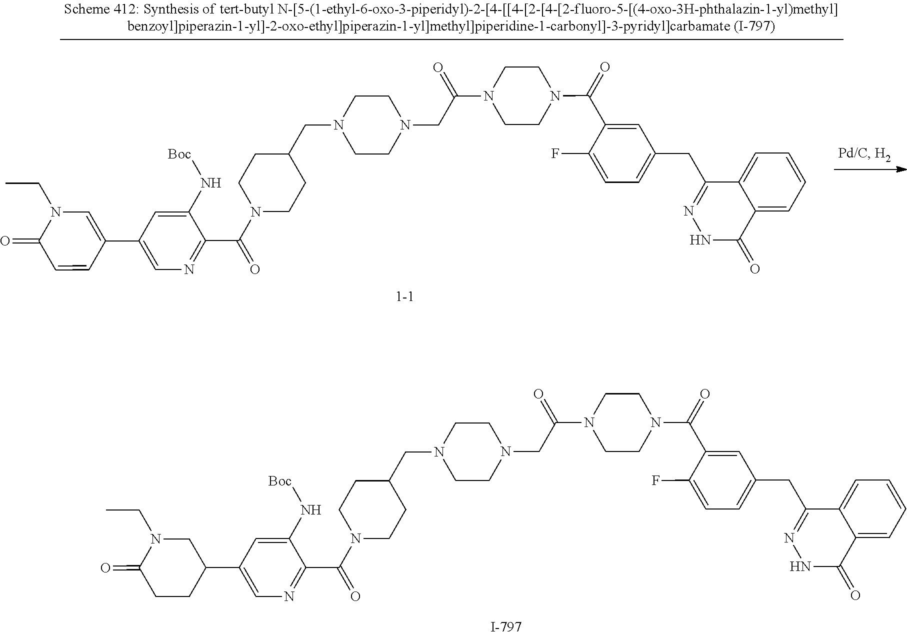 [SMILES: CCn1cc(-c2cnc(C(=O)N3CCC(CN4CCN(CC(=O)N5CCN(C(=O)c6cc(Cc7n[nH]c(=O)c8ccccc78)ccc6F)CC5)CC4)CC3)c(NC(=O)OC(C)(C)C)c2)ccc1=O ; CCN1CC(c2cnc(C(=O)N3CCC(CN4CCN(CC(=O)N5CCN(C(=O)c6cc(Cc7n[nH]c(=O)c8ccccc78)ccc6F)CC5)CC4)CC3)c(NC(=O)OC(C)(C)C)c2)CCC1=O]

To a mixture of Intermediate 1-1 (160 mg, 171.85 μmol, 1 eq) in DMF (2 mL) was added Pd/C (91.44 mg, 85.92 μmol, 10% purity, 0.5 eq). The suspension was degassed and purged with N₂ 3 times, and then the mixture was degassed and purged with H₂ 3 times, then the mixture was stirred at 100° C. for 1 hour under H₂ atmosphere (15 psi). The reaction mixture was filtered and concentrated under reduced pressure to give crude I-797 (120 mg, 128.33 μmol, 74.68% yield) as a white solid. I-797 (30 mg, 32.08 μmol, 1 eq) was purified by reversed-phase HPLC (0.1% NH₃·H₂O condition). The eluent was concentrated under reduced pressure to remove MeCN and then lyophilized to give I-797 (11.86 mg, 12.56 μmol, 39.14% yield, 99% purity) as a yellow solid. LCMS (Method D): Retention time: 0.342 min, (M+H)=935.4. SFC: Rt=4.246 min, Rt=5.775 min. ¹H NMR (400 MHz, METHANOL-d₄) δ=8.37 (d, J=8.0 Hz, 1H), 8.28 (d, J=1.6 Hz, 1H), 8.17 (s, 1H), 7.99-7.93 (m, 1H), 7.92-7.80 (m, 2H), 7.48 (d, J=2.4 Hz, 1H), 7.41-7.33 (m, 1H), 7.20-7.13 (m, 1H), 4.71-4.54 (m, 3H), 4.39 (s, 2H), 3.81-3.65 (m, 5H), 3.58-3.44 (m, 6H), 3.28-3.19 (m, 3H), 3.15-3.07 (m, 1H), 2.92-2.82 (m, 1H), 2.63-2.38 (m, 10H), 2.29-2.21 (m, 2H), 2.16-2.08 (m, 2H), 1.95-1.85 (m, 2H), 1.75-1.69 (m, 1H), 1.51 (s, 9H), 1.34-1.26 (m, 2H), 1.16 (t, J=7.2 Hz, 3H). ¹⁹F NMR (376 MHz, METHANOL-d₄) δ=−120.80.

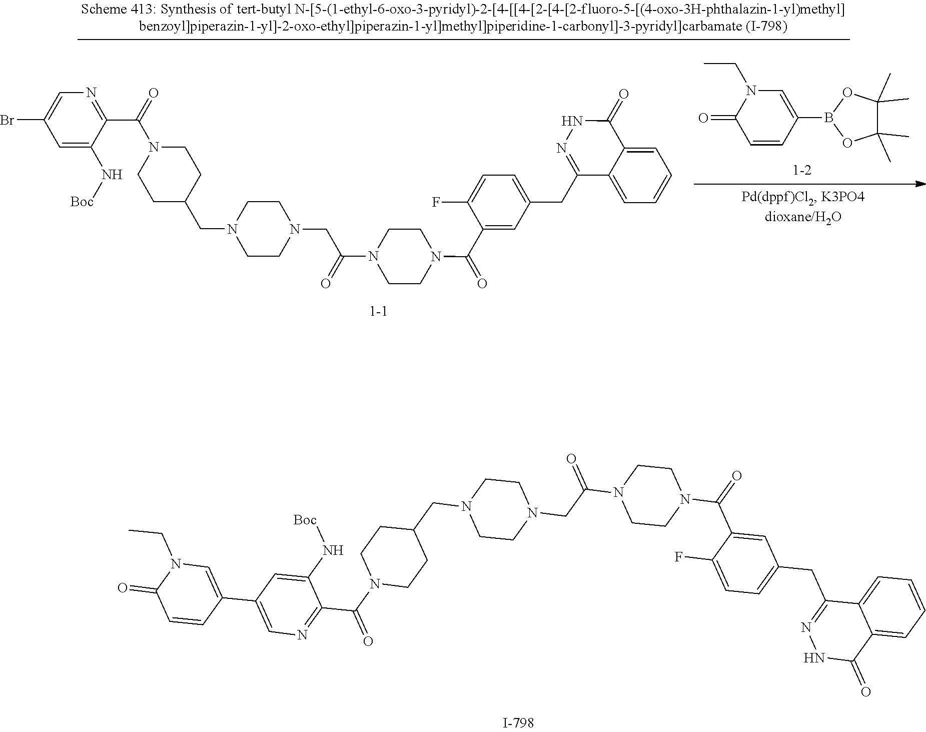 [SMILES: CC(C)(C)OC(=O)Nc1cc(Br)cnc1C(=O)N1CCC(CN2CCN(CC(=O)N3CCN(C(=O)c4cc(Cc5n[nH]c(=O)c6ccccc56)ccc4F)CC3)CC2)CC1 ; CCn1cc(B2OC(C)(C)C(C)(C)O2)ccc1=O ; CCn1cc(-c2cnc(C(=O)N3CCC(CN4CCN(CC(=O)N5CCN(C(=O)c6cc(Cc7n[nH]c(=O)c8ccccc78)ccc6F)CC5)CC4)CC3)c(NC(=O)OC(C)(C)C)c2)ccc1=O]

To a mixture of Intermediate 1-1 (40 mg, 45.00 μmol, 1 eq) and Intermediate 1-2 (33.63 mg, 135.01 μmol, 3 eq) in dioxane (1 mL) and H₂O (0.2 mL) was added Pd(dppf)Cl₂ (3.29 mg, 4.50 μmol, 0.1 eq) and K₃PO₄ (28.66 mg, 135.01 μmol, 3 eq), then the mixture was stirred at 80° C. for 1 hour under N₂. The reaction mixture was diluted with water (30 mL) and extracted with EA (10 mL*2). The combined organic layers were concentrated under reduced pressure to give a residue. The crude product was purified by reversed-phase HPLC (0.1% NH₃·H₂O condition). The eluent was concentrated under reduced pressure to remove MeCN and then lyophilized to give I-798 (16.75 mg, 17.99 μmol, 39.98% yield, 100% purity) as a yellow solid. LCMS (Method D): Retention time: 0.357 min, (M+H)=931.4. ¹H NMR (400 MHz, METHANOL-d₄) δ=8.50 (d, J=2.0 Hz, 1H), 8.40-8.34 (m, 2H), 8.19-8.14 (m, 1H), 7.97-7.82 (m, 4H), 7.53-7.45 (m, 1H), 7.42-7.35 (m, J=6.0, 1H), 7.16 (t, J=9.2 Hz, 1H), 6.68 (d, J=9.6 Hz, 1H), 4.64 (d, J=11.6 Hz, 1H), 4.39 (s, 2H), 4.19-4.11 (m, 2H), 3.81-3.66 (m, 5H), 3.53 (d, J=20.0 Hz, 2H), 3.29-3.26 (m, 2H), 3.20 (s, 1H), 3.16-3.08 (m, 1H), 2.94-2.85 (m, 1H), 2.61-2.37 (m, 8H), 2.27-2.20 (m, 2H), 1.95-1.85 (m, 2H), 1.77-1.70 (m, 1H), 1.53 (s, 9H), 1.42-1.38 (m, 3H), 1.34-1.27 (m, 2H). ¹⁹F NMR (376 MHz, METHANOL-d₄) δ=−120.80.

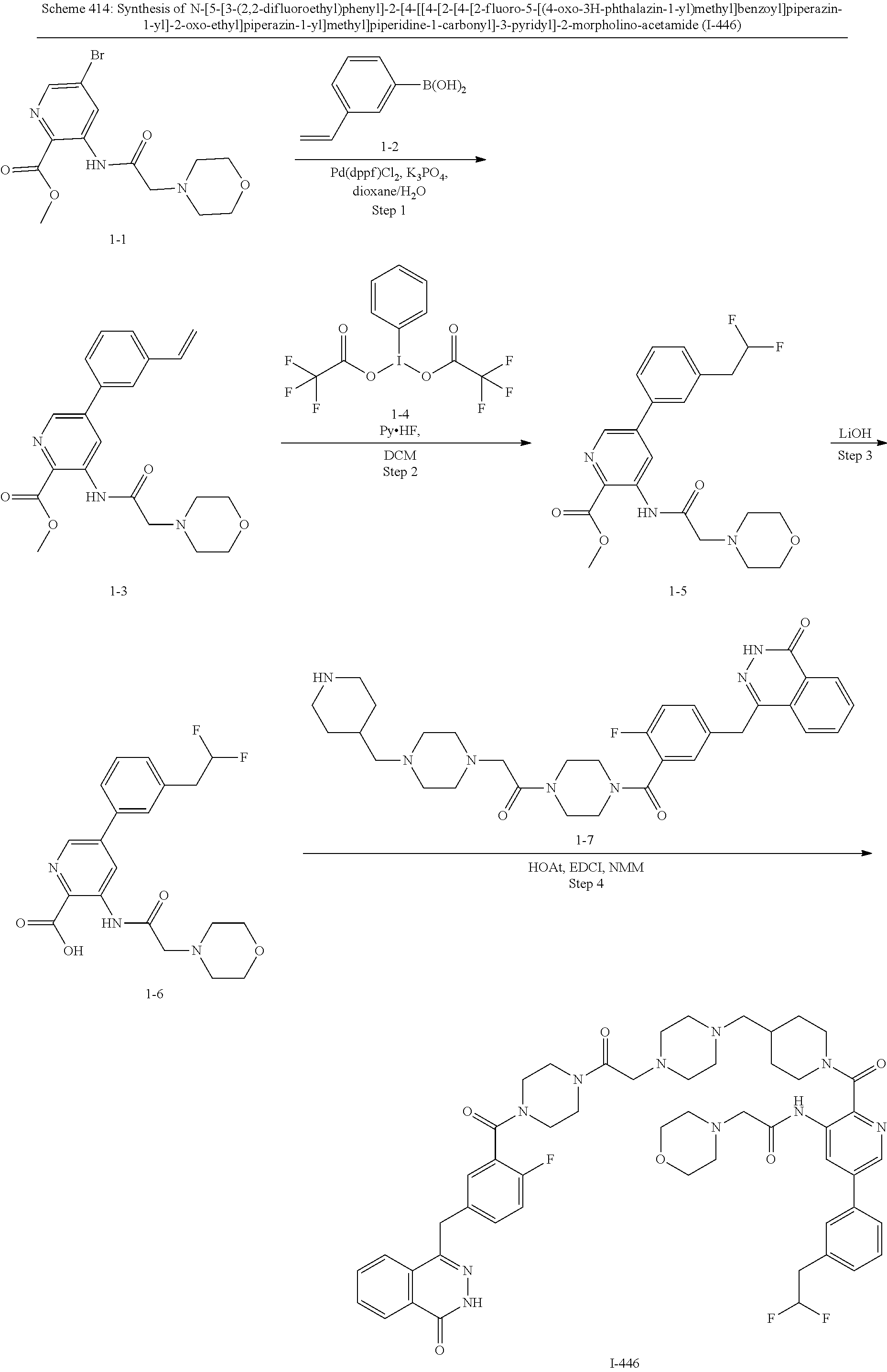

### Step 1: Synthesis of Intermediate 1-3.

To a mixture of Intermediate 1-1 (400 mg, 1.12 mmol, 1 eq) and Intermediate 1-2 (330.48 mg, 2.23 mmol, 2 eq) in dioxane (5 mL) and H₂O (1 mL) was added Pd(dppf)Cl₂ (81.71 mg, 111.67 μmol, 0.1 eq) and K₃PO₄ (711.14 mg, 3.35 mmol, 3 eq), then the mixture was stirred at 80° C. for 1 hour under N2. The reaction mixture was concentrated under reduced pressure to give a residue. The residue was purified by flash silica gel chromatography (ISCO; 40 g SepaFlash Silica Flash Column, Eluent of 0˜50% Ethyl acetate/Petroleum ether gradient @60 mL/min). The eluent was concentrated under reduced pressure to give Intermediate 1-3 (200 mg, 524.35 μmol, 46.95% yield) as a white solid. LCMS (Method D): Retention time: 0.331 min, (M+H)=382.0.

### Step 2: Synthesis of Intermediate 1-5.

To a mixture of Intermediate 1-3 (150 mg, 393.26 μmol, 1 eq) in DCM (10 mL) was added Intermediate 1-4 (186.03 mg, 432.59 μmol, 1.1 eq) and pyridine hydrofluoride (779.49 mg, 7.87 mmol, 708.63 μL, 20 eq), then the mixture was stirred at 20° C. for 1 hour. To the reaction mixture was added a NaHCO₃ solution (about 3 mL) to adjust the pH to 7˜8, and the mixture was extracted with EA (10 mL*3). The combined organic layers were concentrated under reduced pressure to give a residue. The crude product was purified by reversed-phase HPLC (0.1% FA condition). The eluent was concentrated under reduced pressure to remove MeCN and then lyophilized to give Intermediate 1-5 (100 mg, 221.73 μmol, 56.38% yield, 93% purity) as a white solid. LCMS (Method D): Retention time: 0.318 min, (M+H)=420.0. ¹H NMR (400 MHz, CHLOROFORM-d) δ=12.20 (s, 1H), 9.46 (d, J=2.0 Hz, 1H), 8.66 (d, J=2.0 Hz, 1H), 7.62 (d, J=8.0 Hz, 1H), 7.54 (s, 1H), 7.49 (t, J=7.6 Hz, 1H), 7.36 (d, J=7.6 Hz, 1H), 6.18-5.82 (m, 1H), 4.08 (s, 3H), 3.95-3.90 (m, 4H), 3.31-3.20 (m, 4H), 2.69 (s, 4H).

### Step 3: Synthesis of Intermediate 1-6.

To a mixture of Intermediate 1-5 (60 mg, 143.05 μmol, 1 eq) in THF (1 mL), MeOH (0.5 mL) and H₂O (0.25 mL) was added LiOH H₂O (7.20 mg, 171.67 μmol, 1.2 eq), then the mixture was stirred at 20° C. for 1 hour. The reaction mixture was concentrated under reduced pressure to remove THF, and 1 N HCl was added (about 3 mL) to adjust the pH to 3-4, and the mixture was extracted with EA (10 mL*3). The combined organic layers were concentrated under reduced pressure to give Intermediate 1-6 (50 mg, 123.34 μmol, 86.22% yield) as a white solid. LCMS (Method D): Retention time: 0.264 min, (M+H)=406.2.

### Step 4: Synthesis of I-446

To a mixture of Intermediate 1-6 (50 mg, 123.34 μmol, 1 eq) and Intermediate 1-7 (72.73 mg, 123.34 μmol, 1 eq) in DMF (1 mL) was added HOAt (16.79 mg, 123.34 μmol, 17.25 μL, 1 eq), EDCI (70.93 mg, 370.01 μmol, 3 eq) and NMM (62.38 mg, 616.68 μmol, 67.80 μL, 5 eq), then the mixture was stirred at 20° C. for 1 hour. The reaction mixture was quenched with water (20 mL) and extracted with EA (5 mL*3). The combined organic layers were concentrated under reduced pressure to give a residue. The crude product was purified by reversed-phase HPLC (0.1% FA condition). The eluent was concentrated under reduced pressure to remove MeCN and then lyophilized to give I-446 (34.19 mg, 34.99 μmol, 28.37% yield, 100% purity) was obtained as a pink solid. LCMS (Method D): Retention time: 0.311 min, (M+H)=977.6. ¹H NMR (400 MHz, METHANOL-d4) δ=8.89 (d, J=2.0 Hz, 1H), 8.61 (d, J=2.0 Hz, 1H), 8.37 (d, J=7.2 Hz, 1H), 8.00-7.92 (m, 1H), 7.91-7.81 (m, 2H), 7.68-7.60 (m, 2H), 7.55-7.46 (m, 2H), 7.43-7.34 (m, 2H), 7.21-7.12 (m, 1H), 6.29-5.89 (m, 1H), 4.78-4.70 (m, 1H), 4.38 (s, 2H), 3.88-3.63 (m, 10H), 3.59-3.49 (m, 2H), 3.40-3.32 (m, 2H), 3.28-3.25 (m, 2H), 3.20 (s, 4H), 3.17-3.10 (m, 1H), 2.98-2.90 (m, 1H), 2.65-2.61 (m, 4H), 2.58-2.42 (m, 7H), 2.30-2.22 (m, 2H), 2.00-1.88 (m, 2H), 1.82-1.73 (m, 1H), 1.31-1.22 (m, 2H). ¹⁹F NMR (376 MHz, METHANOL-d₄) δ=−116.97, δ=−120.73.

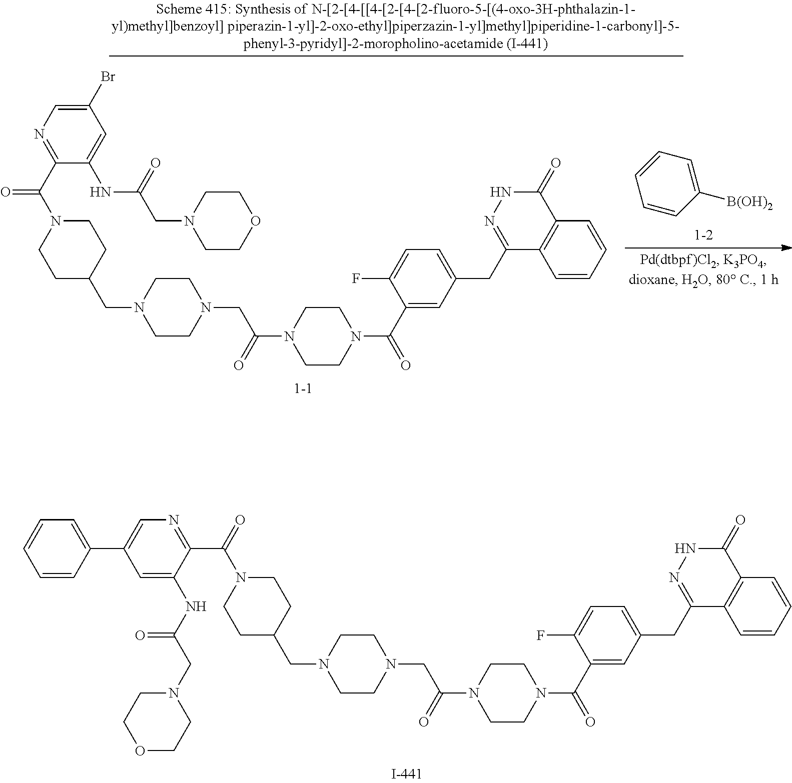 [SMILES: O=C(CN1CCOCC1)Nc1cc(Br)cnc1C(=O)N1CCC(CN2CCN(CC(=O)N3CCN(C(=O)c4cc(Cc5n[nH]c(=O)c6ccccc56)ccc4F)CC3)CC2)CC1 ; OB(O)c1ccccc1 ; O=C(CN1CCOCC1)Nc1cc(-c2ccccc2)cnc1C(=O)N1CCC(CN2CCN(CC(=O)N3CCN(C(=O)c4cc(Cc5n[nH]c(=O)c6ccccc56)ccc4F)CC3)CC2)CC1]

A mixture of intermediate 1-1 (130.00 mg, 141.95 μmol, 1 eq), intermediate 1-2 (19.04 mg, 156.14 μmol, 1.1 eq), Pd(dtbpf)Cl₂ (9.25 mg, 14.19 μmol, 0.1 eq) and K₃PO₄ (90.39 mg, 425.84 μmol, 3 eq) in dioxane (1 mL) and H₂O (0.2 mL) was degassed and purged with N₂ 3 times, and then the mixture was stirred at 80° C. for 1 hr under N₂ atmosphere. The reaction mixture was concentrated under reduced pressure to give a residue. The residue was purified by prep-HPLC (column: Phenomenex Luna C18 150*30 mm*5 um; mobile phase: [water(FA)-ACN]; gradient: 12%-42% B over 10 min) followed by lyophilization to give I-441 (29.05 mg, 28.60 μmol, 20.15% yield, 94.423% purity, FA salt) as a yellow solid. LCMS (Method D): Rt=0.299 min, (M+H)=913.5. ¹H NMR (400 MHz, METHANOL-d₄) δ=8.91-8.84 (m, 1H), 8.64-8.59 (m, 1H), 8.39-8.30 (m, 2H), 7.98-7.91 (m, 1H), 7.90-7.80 (m, 2H), 7.70 (d, J=7.2 Hz, 2H), 7.55-7.49 (m, 3H), 7.48-7.43 (m, 1H), 7.41-7.33 (m, 1H), 7.17 (t, J=9.0 Hz, 1H), 4.75 (d, J=12.4 Hz, 1H), 4.38 (s, 2H), 3.93-3.86 (m, 1H), 3.85-3.78 (m, 6H), 3.75 (s, 1H), 3.72-3.58 (m, 3H), 3.55-3.48 (m, 3H), 3.46 (s, 1H), 3.34 (s, 1H), 3.20 (s, 2H), 3.19-3.12 (m, 1H), 2.99-2.80 (m, 8H), 2.66-2.61 (m, 6H), 2.10-1.94 (m, 2H), 1.78 (d, J=12.0 Hz, 1H), 1.42-1.25 (m, 2H). ¹⁹F NMR (376 MHz, METHAN OL-d₄) δ=−120.764.

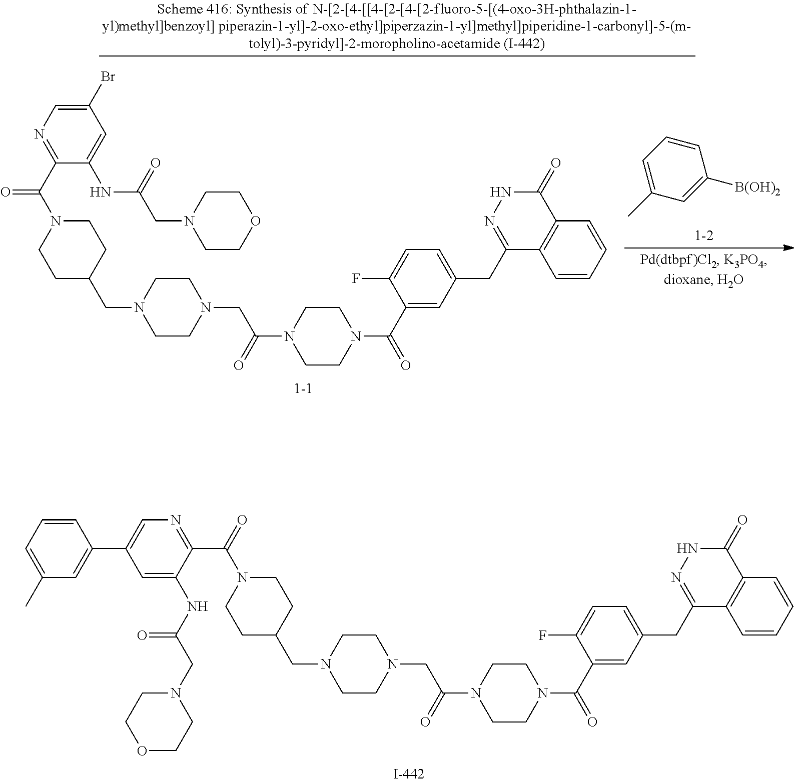

To a solution of intermediate 1-1 (100 mg, 109.19 μmol, 1 eq) and intermediate 1-1 (17.81 mg, 131.03 μmol, 1.2 eq) in H₂O (0.2 mL) and dioxane (1 mL) was added K₃PO₄ (69.53 mg, 327.57 μmol, 3 eq) and Pd(dtbpf)Cl₂ (7.12 mg, 10.92 μmol, 0.1 eq). The mixture was stirred at 80° C. for 2 hr. The reaction mixture was concentrated under reduced pressure to give a residue. The crude product was purified by reversed-phase HPLC (column: Phenomenex Luna C18 150*30 mm*5 um; mobile phase: [water(FA)-ACN]; gradient: 16%-46% B over 10 min) and lyophilized to give product. I-442 (17.55 mg, 17.75 μmol, 16.25% yield, 98.398% purity, FA salt) was obtained as a yellow solid. LCMS (Method E): Rt=0.345 min, (M+H)=927.6. ¹H NMR (400 MHz, METHANOL-d₄) δ=8.84 (d, J=1.6 Hz, 1H), 8.59 (d, J=1.6 Hz, 1H), 8.40-8.29 (m, 2H), 7.99-7.92 (m, 1H), 7.90-7.81 (m, 2H), 7.53-7.47 (m, 3H), 7.43-7.34 (m, 2H), 7.29 (d, J=7.6 Hz, 1H), 7.20-7.13 (m, 1H), 4.79-4.71 (m, 2H), 4.65-4.51 (m, 1H), 4.39 (s, 2H), 3.91-3.85 (m, 1H), 3.84-3.79 (m, 5H), 3.75 (d, J=4.4 Hz, 1H), 3.67 (s, 2H), 3.50 (d, J=13.6 Hz, 3H), 3.40 (s, 1H), 3.20 (s, 2H), 3.18-3.12 (m, 1H), 2.98-2.91 (m, 1H), 2.87-2.69 (m, 8H), 2.64-2.60 (m, 4H), 2.58-2.50 (m 2H), 2.44 (s, 3H), 2.05-1.95 (m, 2H), 1.82-1.73 (m, 1H), 1.37-1.28 (m, 2H). ¹⁹F NMR (376 MHz, METHANOL-d₄) δ=−120.80.

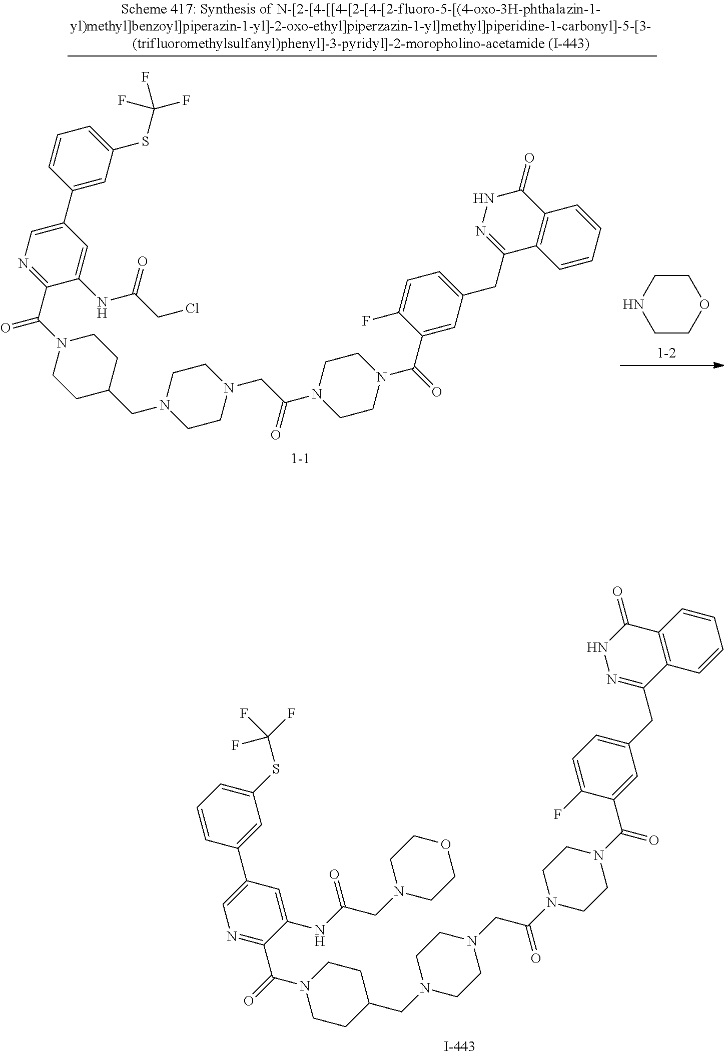 [SMILES: O=C(CCl)Nc1cc(-c2cccc(SC(F)(F)F)c2)cnc1C(=O)N1CCC(CN2CCN(CC(=O)N3CCN(C(=O)c4cc(Cc5n[nH]c(=O)c6ccccc56)ccc4F)CC3)CC2)CC1 ; C1COCCN1 ; O=C(CN1CCOCC1)Nc1cc(-c2cccc(SC(F)(F)F)c2)cnc1C(=O)N1CCC(CN2CCN(CC(=O)N3CCN(C(=O)c4cc(Cc5n[nH]c(=O)c6ccccc56)ccc4F)CC3)CC2)CC1]

To a solution of Intermediate 1-1 (40 mg, 41.56 μmol, 1 eq) and Intermediate 1-2 (5.43 mg, 62.34 μmol, 5.49 μL, 1.5 eq) in ACN (1 mL) was added DIEA (21.49 mg, 166.24 μmol, 28.96 μL, 4 eq) and KI (3.45 mg, 20.78 μmol, 0.5 eq). The mixture was stirred at 40° C. for 1 hr. The reaction mixture was concentrated under reduced pressure to give a residue. The residue was purified by prep-HPLC (column: Welch Xtimate C18 150*30 mm*5 um; mobile phase: [water(FA)-ACN]; gradient: 15%-45% B over 10 min). The eluent was lyophilized to give product. I-443 (19 mg, 17.06 μmol, 41.06% yield, 95.117% purity, FA salt) was obtained as a white solid. LCMS (Method D): Rt=0.375 min, M+H=1013.4. ¹H NMR (400 MHz, METHANOL-d₄) δ=8.90 (d, J=2.0 Hz, 1H), 8.63 (d, J=2.0 Hz, 1H), 8.42 (s, 1H), 8.39-8.34 (m, 1H), 8.03 (s, 1H), 7.93 (t, J=7.6 Hz, 2H), 7.90-7.78 (m, 3H), 7.70-7.64 (m, 1H), 7.52-7.47 (m, 1H), 7.40-7.34 (m, 1H), 7.17 (t, J=8.8 Hz, 1H), 4.75 (d, J=13.2 Hz, 1H), 4.39 (s, 2H), 3.88 (d, J=13.2 Hz, 1H), 3.84-3.78 (m, 5H), 3.77-3.65 (m, 3H), 3.52 (s, 2H), 3.45 (s, 1H), 3.39-3.33 (m, 2H), 3.21 (s, 2H), 3.18-3.11 (m, 1H), 2.99-2.90 (m, 1H), 2.83-2.67 (m, 7H), 2.65-2.61 (m, 4H), 2.48 (d, J=7.2 Hz, 2H), 1.98 (d, J=11.2 Hz, 2H), 1.78 (d, J=12.0 Hz, 1H), 1.41-1.23 (m, 3H). ¹⁹F NMR (376 MHz, METHANOL-d₄) δ=−44.43, −120.79.

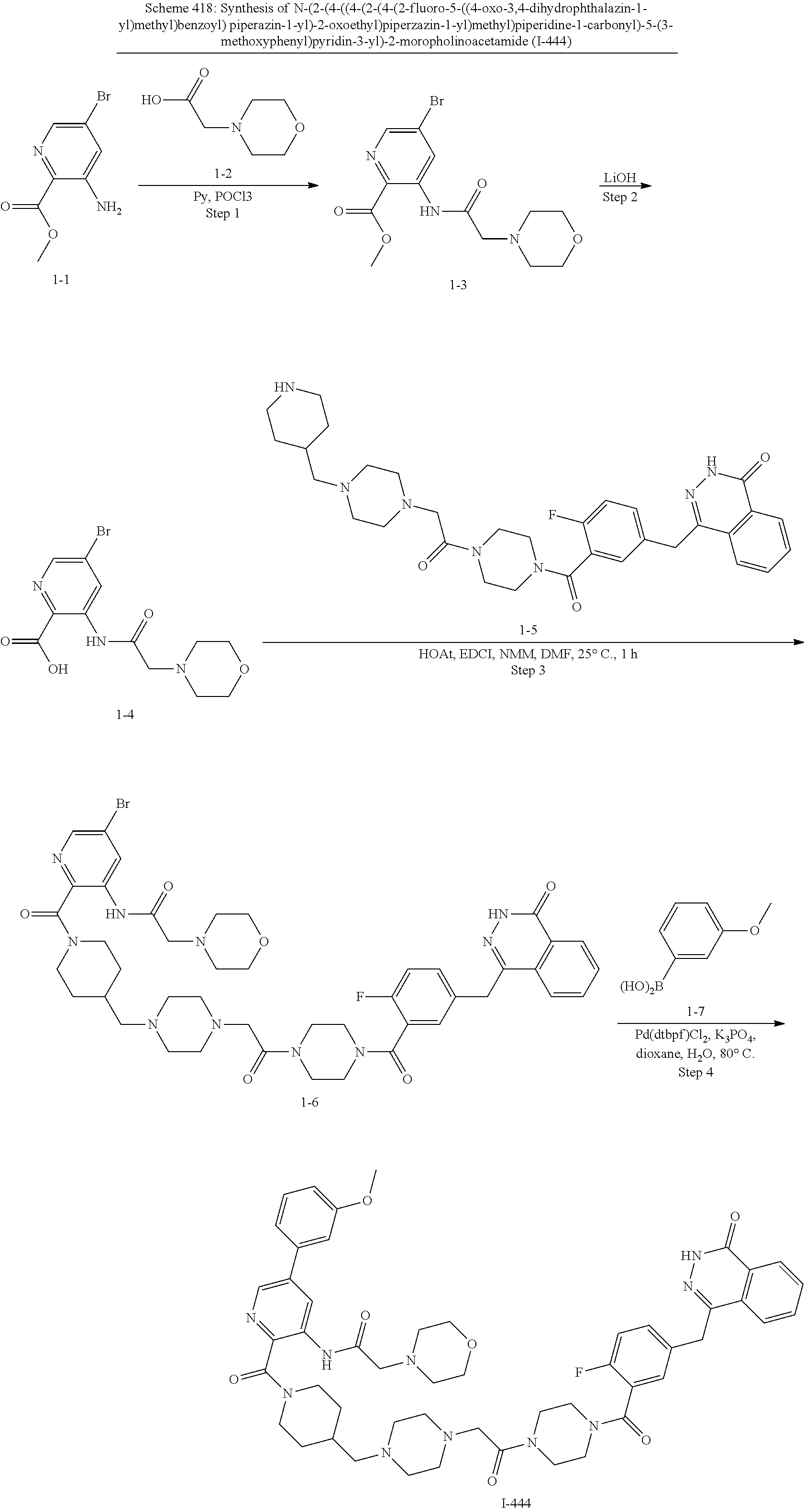

### Step 1: Synthesis of Intermediate 1-3

To a solution of Intermediate 1-2 (3.77 g, 25.97 mmol, 1 eq) and Intermediate 1-1 (6 g, 25.97 mmol, 1 eq) in pyridine (30 mL) was added POCl₃ (4.78 g, 31.16 mmol, 2.90 mL, 1.2 eq). The mixture was stirred at 25° C. for 1 hr. The reaction mixture was added into H₂O (25 mL) at 25° C., and then extracted with EA (10 mL*3). The combined organic layers dried over Na₂SO₄, filtered and concentrated under reduced pressure to give a residue. The residue was purified by flash silica gel chromatography (ISCO; 120 g SepaFlash Silica Flash Column, Eluent of 0-100% Ethyl acetate/Petroleum ether gradient @200 mL/min). Rf=0.51 (EA). Intermediate 1-3 (4.5 g, 12.56 mmol, 48.38% yield, 100% purity) was obtained as a white solid. LCMS (Method E): Rt=0.233 min, [M+H]⁺=357.9.

### Step 2: Synthesis of Intermediate 1-4

To a solution of methyl Intermediate 1-3 (500 mg, 1.40 mmol, 1 eq) in MeOH (2 mL), THF (2 mL) and H₂O (2 mL) was added LiOH·H₂O (175.73 mg, 4.19 mmol, 3 eq). The mixture was stirred at 25° C. for 1 hr. The mixture was concentrated under reduced pressure to give Intermediate 1-4 (600 mg, crude) as a white solid.

### Step 3: Synthesis of Intermediate 1-6

To a solution of Intermediate 1-4 (200 mg, 581.12 μmol, 1 eq) in DMF (20 mL) was added HOAt (79.10 mg, 581.12 μmol, 81.29 μL, 1 eq), NMM (293.89 mg, 2.91 mmol, 319.45 μL, 5 eq) and EDCI (334.21 mg, 1.74 mmol, 3 eq), then Intermediate 1-5 (342.69 mg, 581.12 μmol, 1 eq) was added to the solution. The mixture was stirred at 25° C. for 1 hr. The reaction mixture was quenched by addition of H₂O (10 mL) at 25° C., and then diluted with EA (10 mL) and extracted with EA (10 mL*3), dried by Na₂SO₄, filtered and concentrated under reduced pressure to give a residue. The residue was purified by flash silica gel chromatography (ISCO; 40 g SepaFlash Silica Flash Column, Eluent of 0˜20% DCM/MeOH gradient @100 mL/min). Rf=0.34 (DCM/MeOH=10/1). Intermediate 1-7 was obtained as a colorless oil. LCMS (Method E): Rt=0.275 min, [M+H]⁺=917.1.

### Step 4: Synthesis of I-444

A mixture of Intermediate 1-7 (12.44 mg, 81.89 μmol, 1.5 eq), Intermediate 1-6 (50 mg, 54.59 μmol, 1 eq), K₃PO₄ (34.77 mg, 163.78 μmol, 3 eq), and Pd(dtbpf)Cl₂ (7.99 mg, 10.92 μmol, 0.2 eq) in dioxane (1 mL) and H₂O (0.2 mL) was degassed and purged with N₂ 3 times, and then the mixture was stirred at 80° C. for 1 hr under N₂ atmosphere. The reaction mixture was concentrated under reduced pressure to remove dioxane. The residue was purified by prep-HPLC (column: CD02-Waters Xbidge BEH C18 150*25*10 um; mobile phase: [water (NH₄HCO₃)-ACN]; gradient: 23%-53% B over 10 min). I-444 (13.93 mg, 14.77 μmol, 27.06% yield, 100% purity) was obtained as a yellow solid. LCMS (Method E): Rt=0.314 min, [M+H]⁺=943.5. ¹H NMR (400 MHz, METHANOL-d₄) δ=8.89 (d, J=1.6 Hz, 1H), 8.61 (d, J=2.0 Hz, 1H), 8.39 (d, J=7.2 Hz, 1H), 8.00-7.93 (m, 1H), 7.92-7.82 (m, 2H), 7.50 (s, 1H), 7.47-7.47 (m, 1H), 7.47-7.42 (m, 1H), 7.41-7.35 (m, 1H), 7.30-7.26 (m, 1H), 7.25 (d, J=2.0 Hz, 1H), 7.21-7.14 (m, 1H), 7.06-7.01 (m, 1H), 4.75 (d, J=13.2 Hz, 1H), 4.42-4.37 (m, 1H), 4.40 (s, 1H), 3.89 (s, 3H), 3.86-3.82 (m, 5H), 3.78-3.72 (m, 2H), 3.70-3.66 (m, 1H), 3.60-3.55 (m, 1H), 3.55-3.55 (m, 1H), 3.53 (s, 1H), 3.39-3.36 (m, 1H), 3.28 (s, 1H), 3.22 (s, 3H), 3.16 (s, 1H), 3.01-2.90 (m, 1H), 2.66-2.62 (m, 4H), 2.54 (br s, 8H), 2.30-2.20 (m, 2H), 2.02-1.89 (m, 2H), 1.78 (d, J=11.2 Hz, 1H), 1.34-1.19 (m, 2H). ¹⁹F NMR (377 MHz, METHANOL-d₄) δ=−120.79.

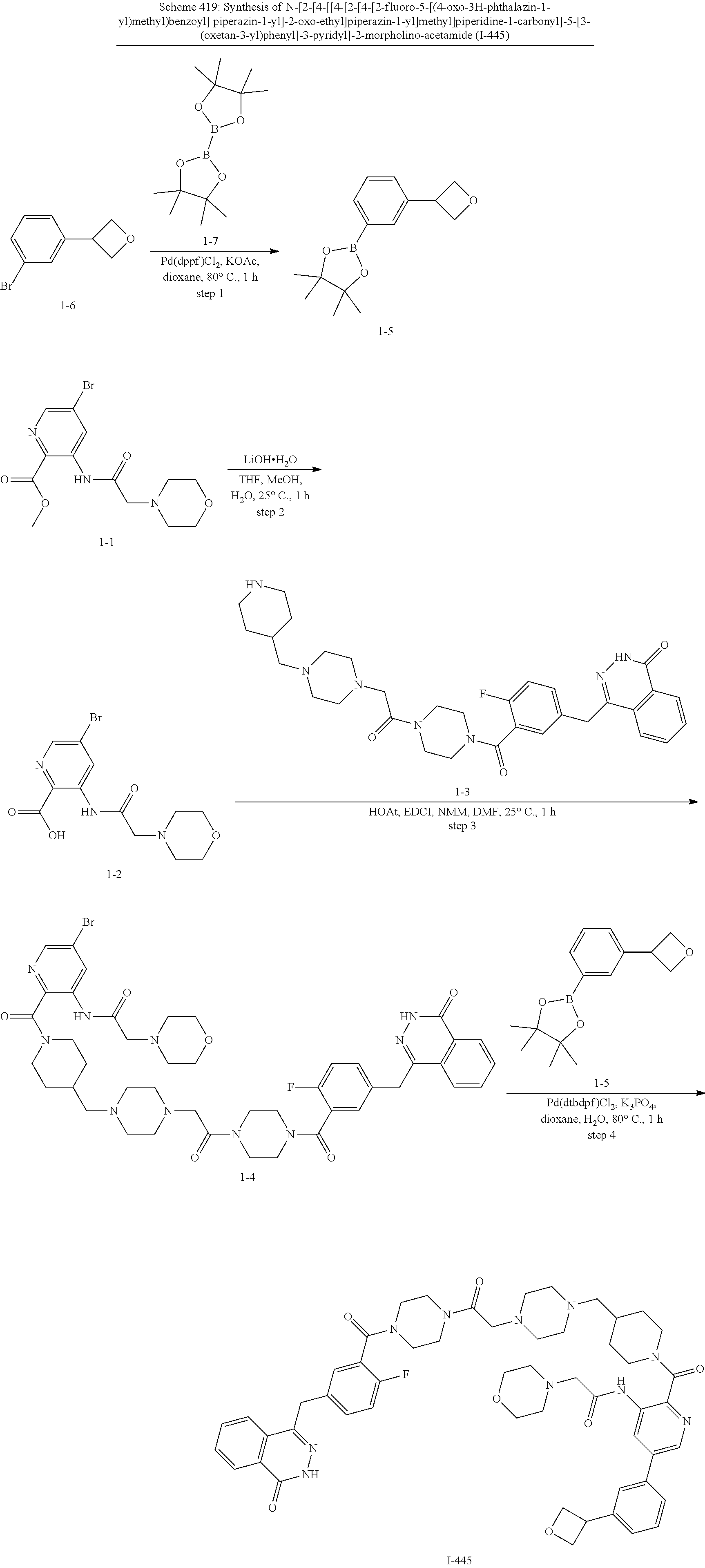

### Step 1: Synthesis of Intermediate 1-5

A mixture of intermediate 1-6 (100 mg 469.33 μmol, 1 eq), intermediate 1-7 (119.18 mg, 469.33 μmol, 1 eq), Pd(dppf)Cl₂ (34.34 mg, 46.93 μmol, 0.1 eq) and KOAc (138.18 mg, 1.41 mmol, 3 eq) in dioxane (1 mL) was degassed and purged with N₂ 3 times, and then the mixture was stirred at 80° C. for 1 hr under N₂ atmosphere. The mixture was concentrated under reduced pressure to give a residue. The residue was purified by flash silica gel chromatography (ISCO; 12 g SepaFlash Silica Flash Column, Eluent of 0-100% Ethylacetate/Petroleum ether gradient @40 mL/min) and concentrated under reduced pressure to give a residue. Intermediate 1-5 (100 mg, 384.41 μmol, 81.91% yield) was obtained as a brown oil. LCMS (Method D): Rt=0.563 min, (M+H)=261.1.

### Step 2: Synthesis of Intermediate 1-2

To a solution of intermediate 1-1 (1 g, 2.79 mmol, 1 eq) in THF (4 mL), MeOH (4 mL) and H₂O (2 mL) was added LiOH H₂O (585.78 mg, 13.96 mmol, 5 eq). The mixture was stirred at 25° C. for 1 hr. The reaction mixture was concentrated under reduced pressure to give a residue. The crude product was used in the next step without further purification. Intermediate 1-2 (950 mg, crude) was obtained as a white solid. LCMS (Method D): Rt=0.124 min, (M+H)=344.0.

### Step 3: Synthesis of Intermediate 1-4

To a solution of intermediate 1-2 (950 mg, 2.76 mmol, 1 eq) and intermediate 1-3 (1.63 g, 2.76 mmol, 1 eq) in DMF (10 mL) was added EDCI (1.59 g, 8.28 mmol, 3 eq), NMM (1.40 g, 13.80 mmol, 1.52 mL, 5 eq) and HOAT (375.71 mg, 2.76 mmol, 386.14 μL, 1 eq). The mixture was stirred at 25° C. for 1 hr. The reaction mixture was concentrated under reduced pressure to give a residue. The residue was purified by flash silica gel chromatography (ISCO; 40 g SepaFlash Silica Flash Column, Eluent of 0˜60% Ethyl acetate/Methanol gradient @60 mL/min) and concentrated under reduced pressure to give a residue. Intermediate 1-4 (500 mg, crude) was obtained as a yellow oil. LCMS (Method D): Rt=0.276 min, (M+H)=915.3.

### Step 4: Synthesis of I-445

A mixture of intermediate 1-4 (130 mg, 141.95 μmol, 1 eq), intermediate 1-5 (40.62 mg, 156.14 μmol, 1.1 eq), Pd(dtbpf)Cl₂ (9.25 mg, 14.19 μmol, 0.1 eq) and K₃PO₄ (90.39 mg, 425.84 μmol, 3 eq) in dioxane (1 mL) and H₂O (0.2 mL) was degassed and purged with N₂ 3 times, and then the mixture was stirred at 80° C. for 1 hr under N₂ atmosphere. The reaction mixture was concentrated under reduced pressure to give a residue. The residue was purified by prep-HPLC (column: Phenomenex Luna C18 150*30 mm*5 um; mobile phase: [water(FA)-ACN]; gradient: 12%-42% B over 10 min) and lyophilized to give a residue. I-445 (28.06 mg, 26.31 μmol, 18.53% yield, 95.165% purity, FA salt) was obtained as a yellow solid. LCMS (Method D): Rt=0.297 min, (M+H)=969.4. ¹H NMR (400 MHz, METHANOL-d₄) δ=8.90-8.84 (m, 1H), 8.63 (d, J=2.0 Hz, 1H), 8.39-8.34 (m, 1H), 8.31 (s, 1H), 7.98-7.91 (m, 1H), 7.91-7.80 (m, 2H), 7.73 (s, 1H), 7.64-7.59 (m, 1H), 7.58-7.53 (m, 2H), 7.51 (d, J=10.0 Hz, 1H), 7.40-7.33 (m, 1H), 7.17 (t, J=9.2 Hz, 1H), 5.17-5.11 (m, 2H), 4.84-4.79 (m, 3H), 4.76 (d, J=13.2 Hz, 1H), 4.43-4.34 (m, 3H), 3.89 (d, J=12.8 Hz, 1H), 3.86-3.78 (m, 6H), 3.75 (d, J=5.6 Hz, 1H), 3.68 (d, J=9.2 Hz, 2H), 3.57-3.45 (m, 4H), 3.34 (d, J=1.6 Hz, 1H), 3.20 (s, 2H), 3.19-3.12 (m, 1H), 2.99-2.81 (m, 8H), 2.68-2.60 (m, 6H), 2.10-1.95 (m, 2H), 1.78 (d, J=12.8 Hz, 1H), 1.43-1.25 (m, 2H). ¹⁹F NMR (376 MHz, METHAN OL-d₄) δ=−120.772.

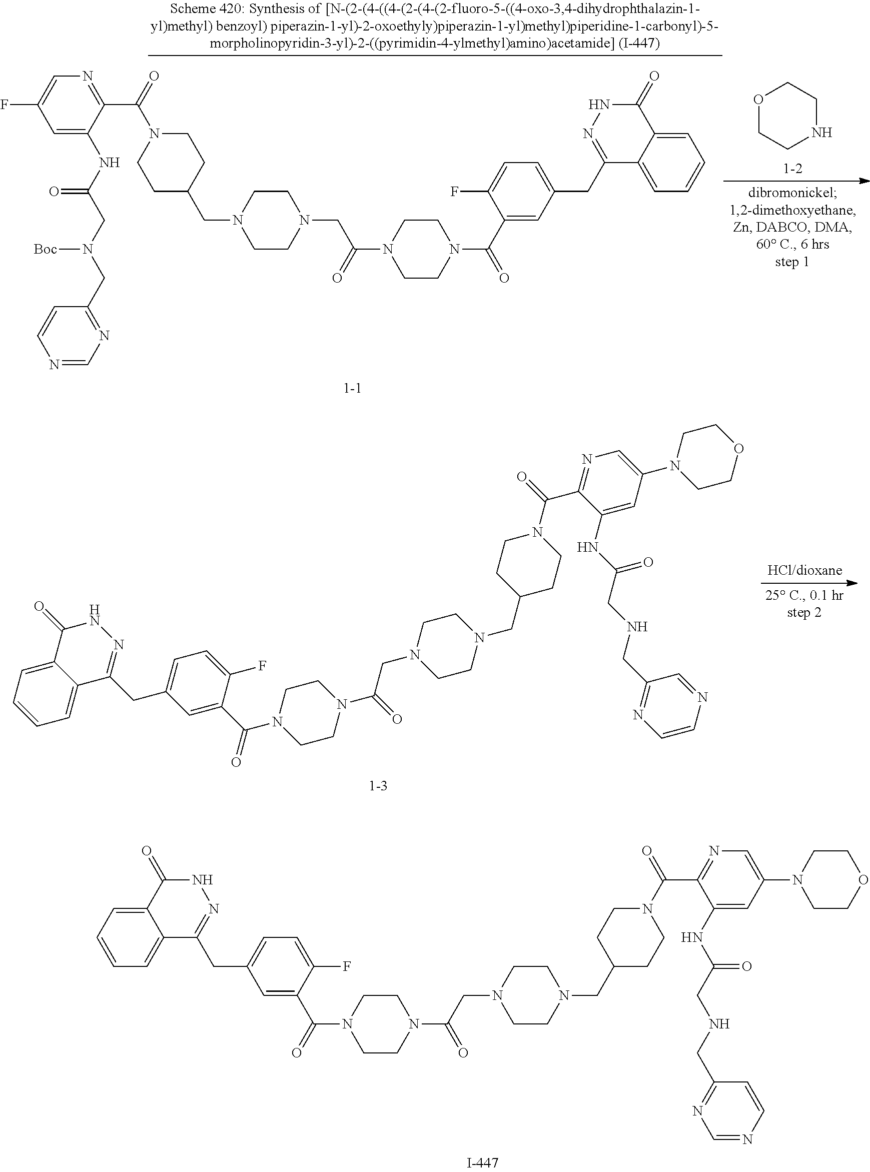 [SMILES: CC(C)(C)OC(=O)N(CC(=O)Nc1cc(F)cnc1C(=O)N1CCC(CN2CCN(CC(=O)N3CCN(C(=O)c4cc(Cc5n[nH]c(=O)c6ccccc56)ccc4F)CC3)CC2)CC1)Cc1ccncn1 ; C1COCCN1 ; O=C(CNCc1cnccn1)Nc1cc(N2CCOCC2)cnc1C(=O)N1CCC(CN2CCN(CC(=O)N3CCN(C(=O)c4cc(Cc5n[nH]c(=O)c6ccccc56)ccc4F)CC3)CC2)CC1 ; O=C(CNCc1ccncn1)Nc1cc(N2CCOCC2)cnc1C(=O)N1CCC(CN2CCN(CC(=O)N3CCN(C(=O)c4cc(Cc5n[nH]c(=O)c6ccccc56)ccc4F)CC3)CC2)CC1]

### Step 1: Synthesis of Intermediate 1-3.

A mixture of Intermediate 1-1 (100 mg, 92.25 μmol, 1 eq, FA salt) and Intermediate 1-2 (32.15 mg, 369.00 μmol, 32.47 μL, 4 eq), DABCO (82.78 mg, 738.01 μmol, 81.16 μL, 8 eq), Ni(DME)Br₂ (28.47 mg, 92.25 μmol, 1 eq) and Zn (12.06 mg, 184.50 μmol, 2 eq) in DMA (1 mL) was degassed and purged with N₂ 3 times, and then the mixture was stirred at 60° C. for 6 hrs under N₂ atmosphere. The reaction mixture was filtered and diluted with H₂O (10 mL) and then extracted with EA (5 mL*3). The combined organic layers dried over sodium sulfate, filtered and concentrated under reduced pressure to give a residue. The solid was treated with 10 mL of 1M HCl. The residue was purified by prep-HPLC (column: CD01-Phenomenex luna C18 150*25*10 um; mobile phase: [water(FA)-ACN]; gradient: 10%-40% B over 10 min), and the eluent was concentrated and lyophilized to give the desired product. Intermediate 1-3 (35 mg, 33.52 μmol, 36.33% yield, 100% purity) was obtained as a white solid. LCMS (Method D): Retention time: 0.344 min, (M+H)=1044.7. ¹H NMR (400 MHz, METHANOL-d₄) δ=9.12-9.06 (m, 1H), 8.74 (d, J=4.8 Hz, 1H), 8.37 (d, J=7.2 Hz, 1H), 8.08 (s, 1H), 7.98-7.93 (m, 1H), 7.91-7.77 (m, 3H), 7.59-7.45 (m, 2H), 7.40-7.33 (m, 1H), 7.20-7.13 (m, 1H), 4.83 (s, 1H), 4.70-4.63 (m, 2H), 4.58 (d, J=12.0 Hz, 1H), 4.39 (s, 2H), 4.25-4.13 (m, 2H), 3.89-3.71 (m, 7H), 3.68 (d, J=4.8 Hz, 2H), 3.61-3.32 (m, 7H), 3.29-3.27 (m, 1H), 3.17-3.06 (m, 1H), 2.88-2.66 (m, 9H), 2.57-2.44 (m, 2H), 2.01-1.83 (m, 2H), 1.77-1.66 (m, 1H), 1.49-1.21 (m, 12H). ¹⁹F NMR (376 MHz, METHANOL-d₄) δ=−120.80.

### Step 2: Synthesis of I-447

To a solution of Intermediate 1-3 (30 mg, 28.73 μmol, 1 eq) in DCM (0.1 mL) was added HCl/dioxane (2 M, 0.1 mL). The mixture was stirred at 25° C. for 0.1 hr. The reaction mixture was concentrated under reduced pressure to give a residue. The residue was dissolved into 1 mL of H₂O, then the solution was lyophilized to give the desired product. I-447 (22.7 mg, 22.82 μmol, 79.42% yield, 98.561% purity, HCl salt) was obtained as a yellow solid. LCMS (Method D): Retention time: 0.260 min, (M+H)=944.5. ¹H NMR (400 MHz, METHANOL-d₄) δ=9.33 (s, 1H), 8.90 (d, J=5.2 Hz, 1H), 8.37 (d, J=7.6 Hz, 1H), 8.27 (d, J=2.4 Hz, 1H), 8.10 (d, J=2.0 Hz, 1H), 8.02-7.92 (m, 1H), 7.91-7.78 (m, 2H), 7.72 (d, J=5.2 Hz, 1H), 7.55-7.46 (m, 1H), 7.42-7.35 (m, 1H), 7.22-7.13 (m, 1H), 4.68 (s, 2H), 4.58 (d, J=17.6 Hz, 2H), 4.37 (d, J=15.6 Hz, 4H), 3.93 (s, 5H), 3.89-3.83 (m, 6H), 3.81-3.71 (m, 3H), 3.66-3.60 (m, 1H), 3.56 (d, J=4.4 Hz, 2H), 3.48-3.44 (m, 4H), 3.41 (s, 2H), 3.36 (d, J=6.4 Hz, 3H), 3.27-3.00 (m, 2H), 2.40-2.27 (m, 1H), 2.10-1.87 (m, 2H), 1.60-1.47 (m, 2H), 1.38-1.21 (m, 2H). ¹⁹F NMR (376 MHz, METHANOL-d₄) δ=−120.75.

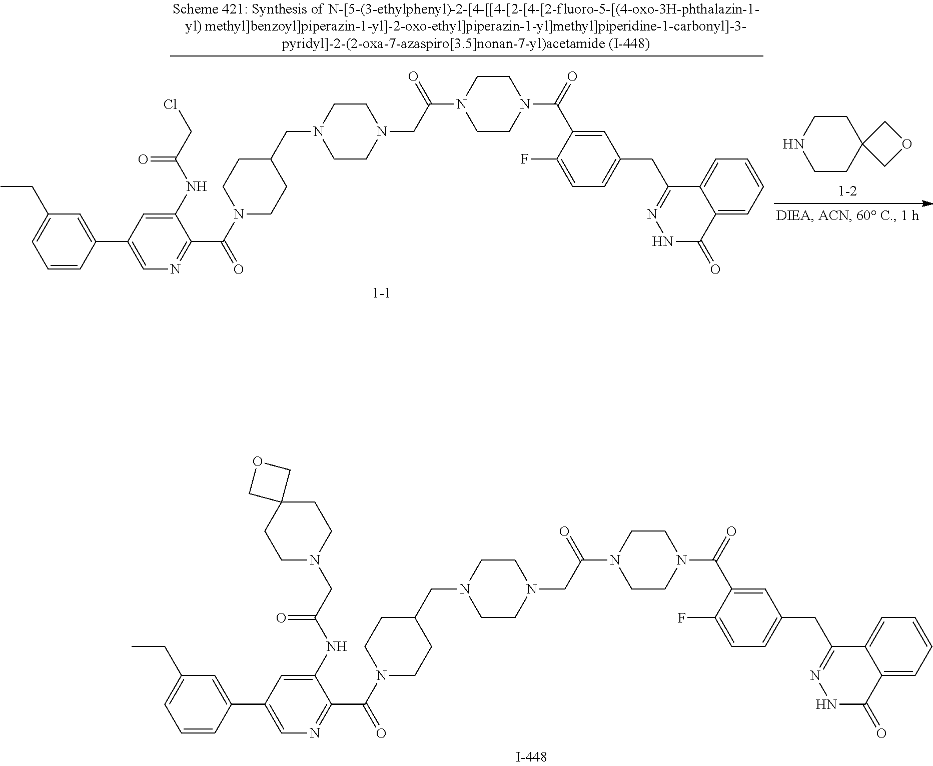 [SMILES: CCc1cccc(-c2cnc(C(=O)N3CCC(CN4CCN(CC(=O)N5CCN(C(=O)c6cc(Cc7n[nH]c(=O)c8ccccc78)ccc6F)CC5)CC4)CC3)c(NC(=O)CCl)c2)c1 ; C1CC2(CCN1)COC2 ; CCc1cccc(-c2cnc(C(=O)N3CCC(CN4CCN(CC(=O)N5CCN(C(=O)c6cc(Cc7n[nH]c(=O)c8ccccc78)ccc6F)CC5)CC4)CC3)c(NC(=O)CN3CCC4(CC3)COC4)c2)c1]

To a solution of intermediate 1-1 (100 mg, 112.30 μmol, 1 eq) in ACN (1 mL) was added DIEA (29.03 mg, 224.61 μmol, 39.12 μL, 2 eq) and intermediate 1-2 (15.71 mg, 123.53 μmol, 1.1 eq). The mixture was stirred at 60° C. for 1 hr. The mixture was concentrated under reduced pressure to give a residue. The crude product was purified by reversed-phase HPLC (0.1% FA condition) and lyophilized to give a residue. I-448 (22 mg, 22.32 μmol, 19.88% yield, 99.550% purity) was obtained as a yellow solid. LCMS (Method D): Rt=0.317 min, (M+H)=981.5. ¹H NMR (400 MHz, METHANOL-d₄) δ=8.83 (d, J=2.0 Hz, 1H), 8.60 (d, J=2.0 Hz, 1H), 8.39-8.35 (m, 1H), 7.99-7.91 (m, 1H), 7.91-7.80 (m, 2H), 7.54-7.47 (m, 3H), 7.43 (t, J=7.6 Hz, 1H), 7.40-7.33 (m, 1H), 7.31 (d, J=7.6 Hz, 1H), 7.17 (t, J=9.2 Hz, 1H), 4.70 (d, J=12.4 Hz, 1H), 4.46 (s, 4H), 4.38 (s, 2H), 3.88-3.72 (m, 3H), 3.72-3.65 (m, 2H), 3.52 (t, J=4.4 Hz, 2H), 3.45 (s, 1H), 3.40-3.33 (m, 2H), 3.27 (s, 1H), 3.21-3.11 (m, 3H), 2.93 (t, J=12.4 Hz, 1H), 2.85-2.64 (m, 10H), 2.52 (s, 5H), 2.46 (s, 1H), 2.02 (t, J=5.2 Hz, 4H), 2.00-1.90 (m, 2H), 1.81-1.73 (m, 1H), 1.29 (t, J=7.6 Hz, 5H). ¹⁹F NMR (376 MHz, METHANOL-d₄) δ=−120.79.

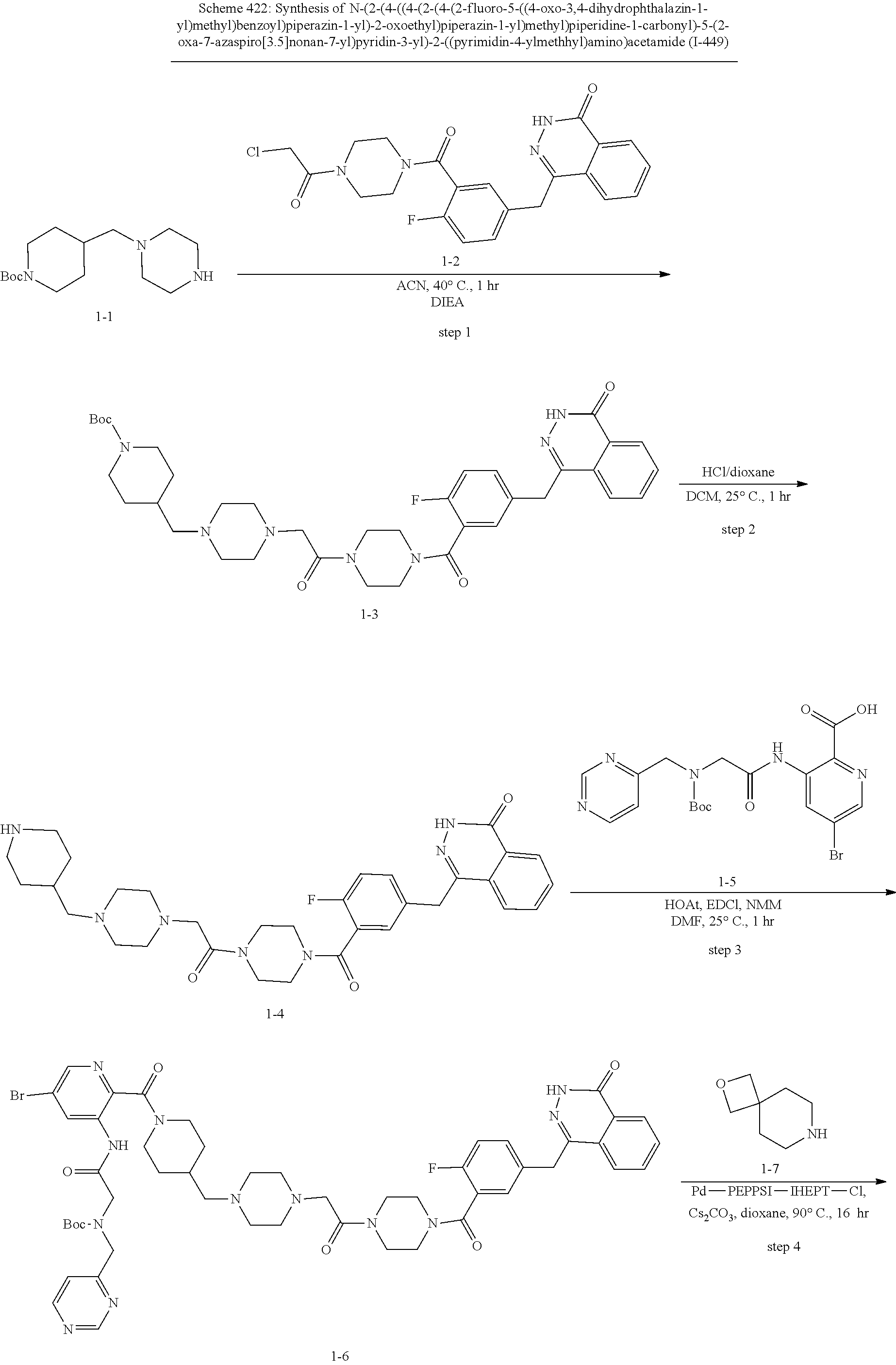

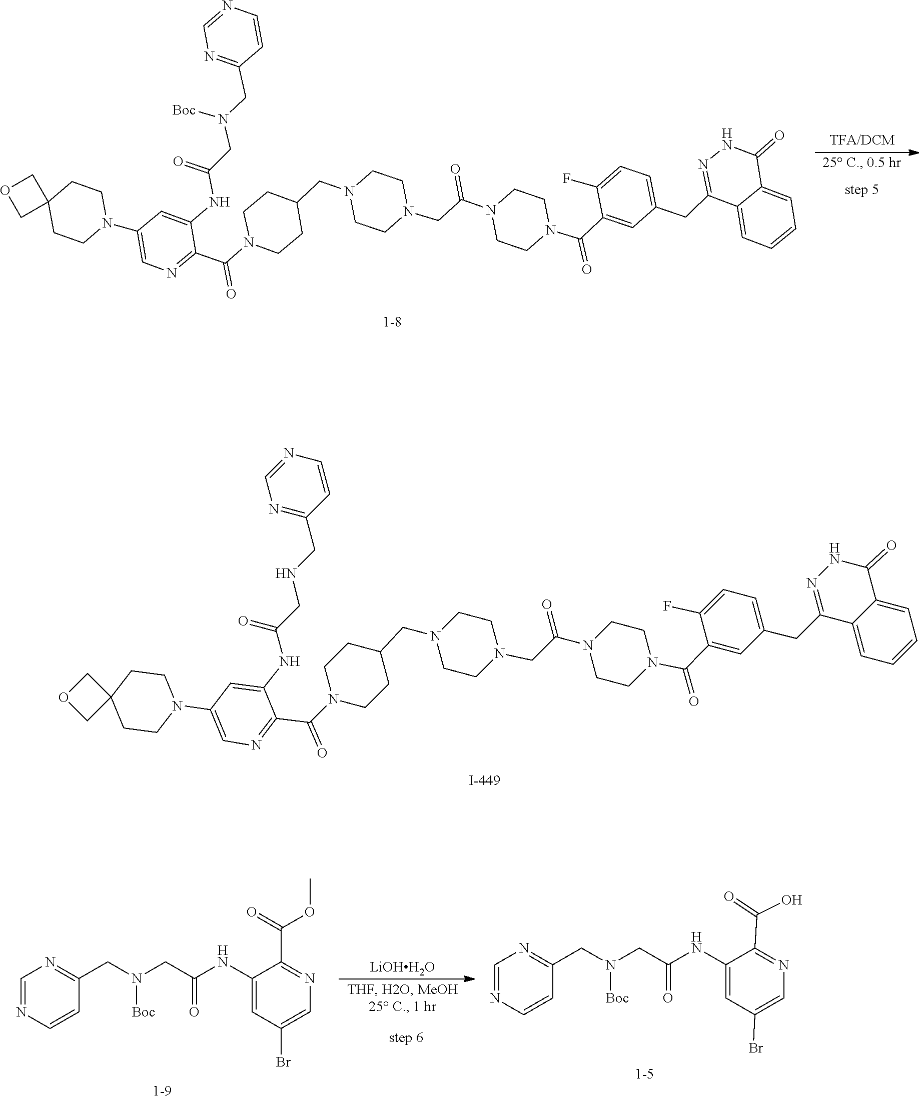 [SMILES: CC(C)(C)OC(=O)N(CC(=O)Nc1cc(N2CCC3(CC2)COC3)cnc1C(=O)N1CCC(CN2CCN(CC(=O)N3CCN(C(=O)c4cc(Cc5n[nH]c(=O)c6ccccc56)ccc4F)CC3)CC2)CC1)Cc1ccncn1 ; O=C(CNCc1ccncn1)Nc1cc(N2CCC3(CC2)COC3)cnc1C(=O)N1CCC(CN2CCN(CC(=O)N3CCN(C(=O)c4cc(Cc5n[nH]c(=O)c6ccccc56)ccc4F)CC3)CC2)CC1 ; COC(=O)c1ncc(Br)cc1NC(=O)CN(Cc1ccncn1)C(=O)OC(C)(C)C ; "LiOH·H2O" ; CC(C)(C)OC(=O)N(CC(=O)Nc1cc(Br)cnc1C(=O)O)Cc1ccncn1]

### Step 1: Synthesis of Intermediate 1-3

To a solution of Intermediate 1-1 (3.30 g, 11.66 mmol, 1 eq) and Intermediate 1-2 (5.16 g, 11.66 mmol, 1 eq) in ACN (50 mL) was added DIEA (4.52 g, 34.98 mmol, 6.09 mL, 3 eq). The mixture was stirred at 40° C. for 1 hr. The mixture was washed with water (100 mL) and extracted with DCM (30 mL*3). The combined organic layers were dried over sodium sulfate, filtered and concentrated under reduced pressure to give a residue. The residue was purified by flash silica gel chromatography (ISCO; 120 g SepaFlash Silica Flash Column, Eluent of 0˜15% EA/MeOH gradient @100 mL/min) and then the eluent was concentrated in vacuo to give Intermediate 1-3 (2.8 g, 3.75 mmol, 32.17% yield, 92.4% purity) as yellow solid. LCMS (Method D): Rt=0.332 min, (M+H)=690.4. ¹H NMR (400 MHz, METHANOL-d4) δ=8.37 (d, J=7.6 Hz, 1H), 8.00-7.91 (m, 1H), 7.91-7.80 (m, 2H), 7.49 (d, J=2.4 Hz, 1H), 7.38 (m, 1H), 7.16 (m, 1H), 4.38 (s, 2H), 4.09-4.02 (m, 2H), 3.80-3.62 (m, 4H), 3.58-3.49 (m, 2H), 3.32-3.22 (m, 5H), 2.75 (s, 2H), 2.53 (s, 8H), 2.22 (m, 2H), 1.74 (d, J=11.6 Hz, 3H), 1.45 (s, 8H), 1.12-0.95 (m, 2H). ¹⁹F NMR (376 MHz, METHANOL-d4) δ=−120.73.

### Step 2: Synthesis of Intermediate 1-4

To a solution of Intermediate 1-3 (2.7 g, 3.91 mmol, 1 eq) in DCM (27 mL) was added HCl/dioxane (2 M, 27 mL). The mixture was stirred at 25° C. for 1 hr. The reaction mixture was concentrated in vacuo. The crude product was used in the next step without further purification. Intermediate 1-4 (3.3 g, crude, HCl salt) was obtained as white solid. LCMS (Method D): Rt=0.266 min, (M+H)=590.3. ¹H NMR (400 MHz, METHANOL-d4) δ=8.36 (d, J=7.6 Hz, 1H), 8.00-7.92 (m, 1H), 7.92-7.80 (m, 2H), 7.50 (m, 1H), 7.44-7.33 (m, 1H), 7.17 (m, 1H), 4.61 (d, J=17.6 Hz, 2H), 4.39 (s, 2H), 4.02-3.82 (m, 8H), 3.70 (s, 3H), 3.61-3.55 (m, 2H), 3.50-3.41 (m, 4H), 3.36 (d, J=6.4 Hz, 2H), 3.08 (m, 2H), 2.35 (s, 1H), 2.22 (d, J=14.0 Hz, 2H), 1.65-1.52 (m, 3H). ¹⁹F NMR (376 MHz, METHANOL-d4) δ=−120.70.

### Step 3: Synthesis of Intermediate 1-6

To a solution of Intermediate 1-5 (300 mg, 643.38 μmol, 1 eq) in DMF (3 mL) was added HOAt (87.57 mg, 643.38 μmol, 90.00 μL, 1 eq) and EDCI (370.01 mg, 1.93 mmol, 3 eq), NMM (325.38 mg, 3.22 mmol, 353.68 μL, 5 eq) and Intermediate 1-6 (402.86 mg, 643.38 μmol, 1 eq, HCl salt). The mixture was stirred at 25° C. for 1 hr. The mixture was washed with water (10 mL) and extracted with EA (5 mL*3). The combined organic layers dried over sodium sulfate, filtered and concentrated under reduced pressure to give a residue. The residue was purified by prep-HPLC (column: CD05-Phenomenex luna C18 150*40*10 um; mobile phase: [water (FA)-ACN]; gradient: 8%-38% B over 11 min) and the eluent was concentrated under reduced pressure to remove ACN and lyophilized to give Intermediate 1-6 (400 mg, 364.07 μmol, 56.59% yield, 98.664% purity, FA salt) as a white solid. LCMS (Method D): Rt=0.373 min, (M+H)=1037.4. ¹H NMR (400 MHz, METHANOL-d4) δ=9.17-9.05 (m, 1H), 8.74 (d, J=4.8 Hz, 1H), 8.54-8.42 (m, 2H), 8.40-8.36 (m, 1H), 8.35 (s, 1H), 8.01-7.93 (m, 1H), 7.89-7.79 (m, 2H), 7.56-7.48 (m, 2H), 7.37 (m, 1H), 7.17 (m, 1H), 4.67 (s, 2H), 4.59 (d, J=13.2 Hz, 1H), 4.39 (s, 2H), 4.26-4.16 (m, 2H), 3.82-3.66 (m, 5H), 3.50 (d, J=14.0 Hz, 4H), 3.40 (s, 2H), 3.15-3.08 (m, 1H), 2.87-2.67 (m, 9H), 2.59-2.51 (m, 2H), 2.01-1.87 (m, 2H), 1.77-1.68 (m, 1H), 1.44 (s, 2H), 1.31 (s, 9H). ¹⁹F NMR (377 MHz, METHANOL-d4) δ=−120.74.

### Step 4: Synthesis of Intermediate 1-8

A mixture of Intermediate 1-6 (90 mg, 86.71 μmol, 0.6 eq), Intermediate 1-7 (73.52 mg, 578.05 μmol, 4 eq), Cs₂CO₃ (141.26 mg, 433.54 μmol, 3 eq), and 1,3-bis[2,6-bis(1-propyl butyl) phenyl]-4,5-dichloro-2H-imidazol-1-ium-2-ide;3-chloropyridine; dichloropalladium (14.06 mg, 14.45 μmol, 0.1 eq) in dioxane (0.8 mL) was degassed and purged with N₂ 3 times, and then the mixture was stirred at 90° C. for 16 hrs under N₂ atmosphere. The mixture was washed with water (1 mL) and extracted with EA (2 mL*3). The combined organic layers were dried over sodium sulfate, filtered and concentrated under reduced pressure to give a residue. The residue was purified by prep-HPLC (FA condition), and the eluent was concentrated under reduced pressure to remove ACN and lyophilized to give Intermediate 1-8 (60 mg, 50.27 μmol, 34.78% yield, 90.835% purity) as a white solid. LCMS (Method D): Rt=0.326 min, (M+H)=1084.5.

### Step 5: Synthesis of I-449

To a solution of Intermediate 1-8 (55 mg, 50.73 μmol, 1 eq) in DCM (0.5 mL) was added TFA (0.1 mL). The mixture was stirred at 25° C. for 0.5 hr. The pH was basified to 7-8 with NH₃·H₂O, and the mixture was washed with water (5 mL) and extracted with EA (8 mL*3). The combined organic layers dried over sodium sulfate, filtered and concentrated under reduced pressure to give a residue. The residue was purified by reversed phase HPLC (neutral condition), and the eluent was concentrated under reduced pressure to remove ACN and lyophilized to give I-449 (15.49 mg, 15.52 μmol, 30.60% yield, 98.634% purity) as a white solid. LCMS (Method H): Rt=0.489 min, (M+H)=984.7. ¹H NMR (400 MHz, METHANOL-d4) δ=9.08 (s, 1H), 8.75 (d, J=5.2 Hz, 1H), 8.38-8.35 (m, 1H), 8.20 (d, J=2.0 Hz, 1H), 8.02 (d, J=2.4 Hz, 1H), 7.98-7.93 (m, 1H), 7.89-7.80 (m, 3H), 7.51-7.46 (m, 1H), 7.37 (m, 1H), 7.16 (m, 1H), 4.60-4.51 (m, 2H), 4.50 (s, 4H), 4.39 (s, 2H), 3.98 (s, 2H), 3.86 (d, J=13.6 Hz, 1H), 3.79 (d, J=2.4 Hz, 1H), 3.73 (s, 2H), 3.67 (d, J=2.0 Hz, 1H), 3.56-3.50 (m, 2H), 3.47 (s, 2H), 3.33 (s, 2H), 3.27 (s, 1H), 3.20 (s, 1H), 3.13-3.05 (m, 1H), 2.83-2.77 (m, 1H), 2.51 (s, 8H), 2.23-2.17 (m, 2H), 2.04-1.96 (m, 5H), 1.93-1.74 (m, 3H), 1.74-1.65 (m, 1H), 1.34-1.01 (m, 3H). ¹⁹F NMR (376 MHz, METHANOL-d4) δ=−120.77.

### Step 6: Synthesis of Intermediate 1-5

To a solution of Intermediate 1-9 (1.4 g, 2.91 mmol, 1 eq) in THF (5 mL), MeOH (5 mL) and H₂O (5 mL) was added LiOH·H₂O (366.94 mg, 8.74 mmol, 3 eq). The mixture was stirred at 25° C. for 1 hr. The pH was acidified to 3-4 with 1N HCl, and the mixture was washed with water (5 mL) and extracted with EA (8 mL*3). The combined organic layers were dried over sodium sulfate, filtered and concentrated under reduced pressure to give a residue. The crude product was used in the next step without further purification. Intermediate 1-5 (1.49 g, crude) was obtained as yellow oil. LCMS (Method D): Rt=0.366 min, (M+H)=466.1. ¹H NMR (400 MHz, METHANOL-d4) δ=9.36 (d, J=8.0 Hz, 1H), 9.10 (s, 1H), 8.76 (d, J=5.2 Hz, 1H), 8.46 (s, 1H), 7.57 (m, 1H), 4.75 (s, 2H), 4.27 (d, J=16.8 Hz, 2H), 1.39 (d, J=14.0 Hz, 9H).

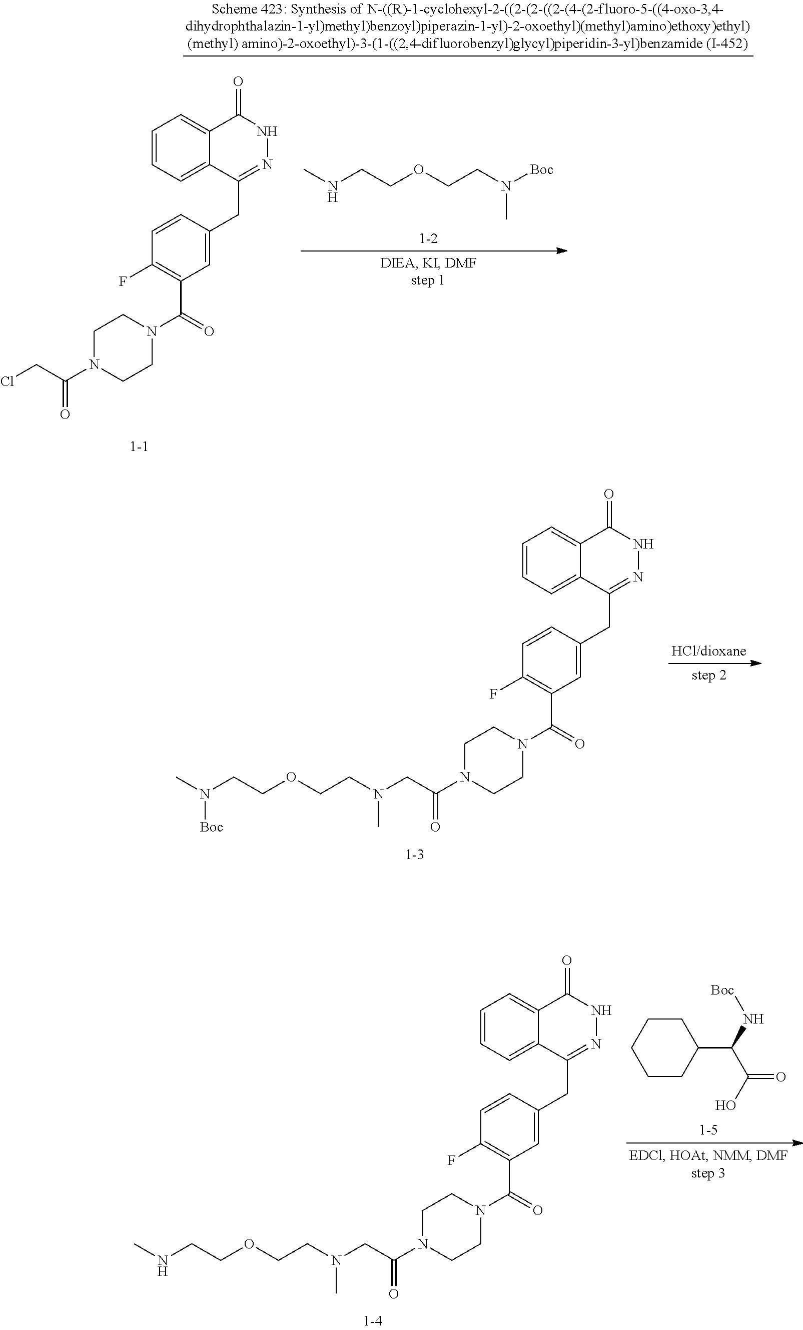

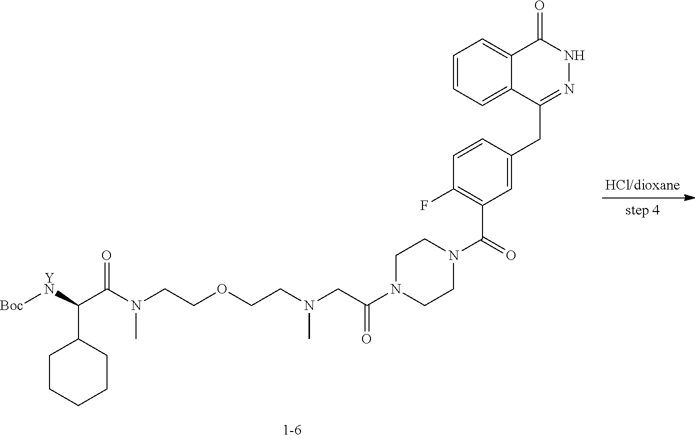

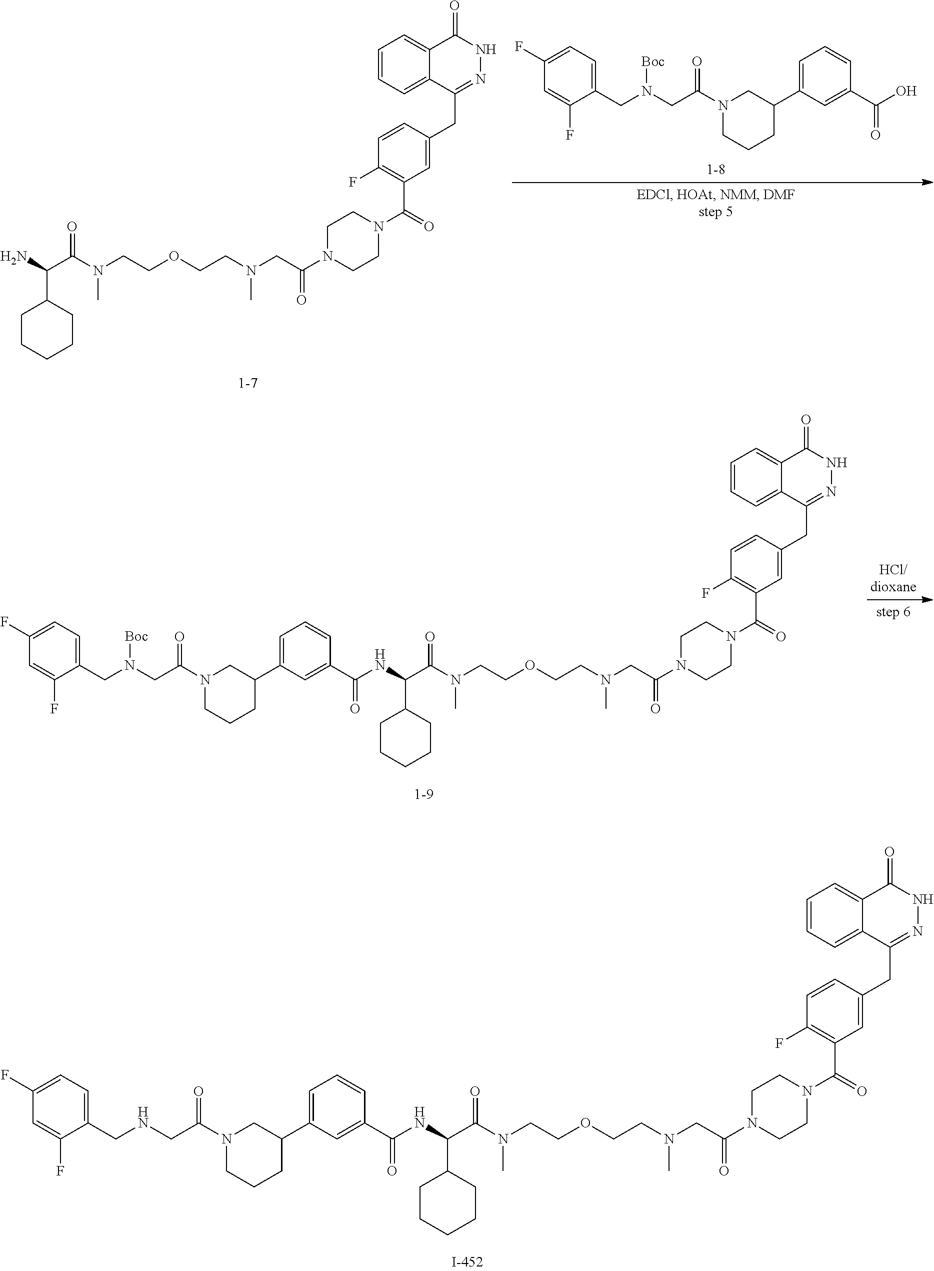

### Step 1: Synthesis of Intermediate 1-3

To a solution of Intermediate 1-1 (250 mg, 564.50 μmol, 1 eq) in DMF (2.5 mL) was added Intermediate 1-2 (131.14 mg, 564.50 μmol, 1 eq), KI (374.83 mg, 2.26 mmol, 4 eq) and DIEA (291.83 mg, 2.26 mmol, 393.30 μL, 4 eq). The mixture was stirred at 25° C. for 1 h. The reaction mixture was quenched slowly by H₂O (4 mL) extracted with EA (2 mL*3). The combined organic layers was washed with brine (2 ml*3), dried over Na₂SO₄, filtered and concentrated under reduced pressure to give a residue. The residue was purified by flash silica gel chromatography (ISCO; 12 g SepaFlashSilica Flash Column, Eluent of 0˜10% DCM/Methanol@100 mL/min). Intermediate 1-3 (350 mg, 473.82 μmol, 83.94% yield, 86.470% purity) was obtained as a yellow oil. LCMS (Method G): Rt=0.557 min, [M+H]⁺=639.5.

### Step 2: Synthesis of Intermediate 1-4

To a solution of Intermediate 1-3 (350 mg, 547.96 μmol, 1 eq) in DCM (1 mL) was added HCl/dioxane (2 M, 2.92 mL, 10.65 eq). The mixture was stirred at 25° C. for 1 h. The mixture was concentrated under vacuum to give a crude product. The crude was used for the next step directly without purification. Intermediate 1-4 (350 mg, crude, HCl salt) was obtained as a yellow solid. LCMS (Method G): Rt=0.515 min, [M+H]⁺=539.3.

### Step 3: Synthesis of Intermediate 1-6

To a solution of Intermediate 1-4 (0.35 g, 608.62 μmol, 1 eq, HCl salt) in DMF (4 mL) was added HOAt (82.84 mg, 608.62 μmol, 85.14 μL, 1 eq) and EDCI (233.35 mg, 1.22 mmol, 2 eq), NMM (307.80 mg, 3.04 mmol, 334.56 μL, 5 eq) and Intermediate 1-5 (156.61 mg, 608.62 μmol, 1 eq). The mixture was stirred at 25° C. for 1 h. The reaction mixture was quenched slowly by H₂O (8 mL), and extracted with EA (3 mL*3). The combined organic layer was washed with brine (3 ml*3), dried over Na₂SO₄, filtered and concentrated under reduced pressure to give a residue. The residue was purified by flash silica gel chromatography (ISCO; 12 g SepaFlashSilica Flash Column, Eluent of 0˜10% DCM/Methanol@100 mL/min). Intermediate 1-6 (280 mg, 336.38 μmol, 55.27% yield, 93.456% purity) was obtained as a yellow oil. LCMS (Method G): Rt=0.629 min, [M+H]⁺=778.5. SFC: Rt=2.198 min. ¹H NMR (400 MHz, CHLOROFORM-d) δ=10.94-9.99 (m, 1H), 8.40 (br d, J=6.3 Hz, 1H), 7.77-7.62 (m, 3H), 7.26 (br s, 2H), 6.98 (br t, J=8.8 Hz, 1H), 4.39 (br s, 1H), 4.22 (s, 2H), 3.74-3.47 (m, 11H), 3.39-3.24 (m, 4H), 3.14-2.98 (m, 3H), 2.67-2.60 (m, 1H), 2.37-2.17 (m, 3H), 1.73-1.50 (m, 9H), 1.39-1.33 (m, 9H), 1.16-0.95 (m, 6H). ¹⁹F NMR (377 MHz, CHLOROFORM-d).

### Step 4: Synthesis of Intermediate 1-7

To a solution of Intermediate 1-6 (140 mg, 179.97 μmol, 1 eq) in DCM (1 mL) was added HCl/dioxane (2 M, 2 mL, 22.23 eq). The mixture was stirred at 25° C. for 1 h. The mixture was concentrated under vacuum to give a crude product. The crude was used for the next step without purification. Intermediate 1-7 (120 mg, crude, HCl salt) was obtained as a yellow solid. LCMS (Method G): Rt=0.550 min, [M+H]⁺=678.5.

### Step 5: Synthesis of Intermediate 1-9

To a solution of Intermediate 1-8 (82.07 mg, 168.00 μmol, 1 eq) in DMF (2 mL) was added HOAt (22.87 mg, 168.00 μmol, 23.50 μL, 1 eq), EDCI (64.41 mg, 336.01 μmol, 2 eq), NMM (84.97 mg, 840.02 μmol, 92.35 μL, 5 eq) and Intermediate 1-7 (120 mg, 168.00 μmol, 1 eq, HCl salt). The mixture was stirred at 25° C. for 1 h. The mixture was poured into H₂O (10 mL), filtered and the filter cake was washed with H₂O (2 mL*3) and dried under vacuum to give a crude product. The crude was used for the next step without purification. Intermediate 1-9 (200 mg, crude) was obtained as a yellow solid. LCMS (Method G): Rt=0.749 min, [M+H]⁺=1149.8.

### Step 6: Synthesis of I-452

To a solution of Intermediate 1-9 (80 mg, 69.67 μmol, 1 eq) in DCM (0.5 mL) was added HCl/dioxane (2 M, 1 mL, 28.71 eq). The mixture was stirred at 25° C. for 1 h. The mixture was concentrated under vacuum to give a residue. The residue was purified by Prep-HPLC (column: Phenomenex luna C18 150*25 mm*10 um; mobile phase: [water (FA)-ACN]; gradient: 10%-40% B over 9 min). I-452 (11.23 mg, 10.71 μmol, 15.38% yield, 100% purity) was obtained as a white solid. LCMS (Method G): Rt=0.664 min, [M+H]⁺=1048.7. SFC: Rt=4.075 min, 6.955 min. ¹H NMR (400 MHz, METHANOL-d₄) δ=8.36 (br d, J=7.2 Hz, 1H), 7.93 (br d, J=7.6 Hz, 1H), 7.90-7.79 (m, 2H), 7.78-7.66 (m, 2H), 7.53-7.33 (m, 5H), 7.21-7.11 (m, 1H), 7.06-6.91 (m, 2H), 5.06-4.89 (m, 1H), 4.58-4.47 (m, 1H), 4.40-4.31 (m, 2H), 3.97-3.87 (m, 2H), 3.85-3.37 (m, 18H), 3.25 (br s, 1H), 3.17-3.07 (m, 1H), 3.01-2.90 (m, 1H), 2.86-2.66 (m, 4H), 2.50-2.35 (m, 3H), 2.08-1.49 (m, 11H), 1.41-1.04 (m, 6H).

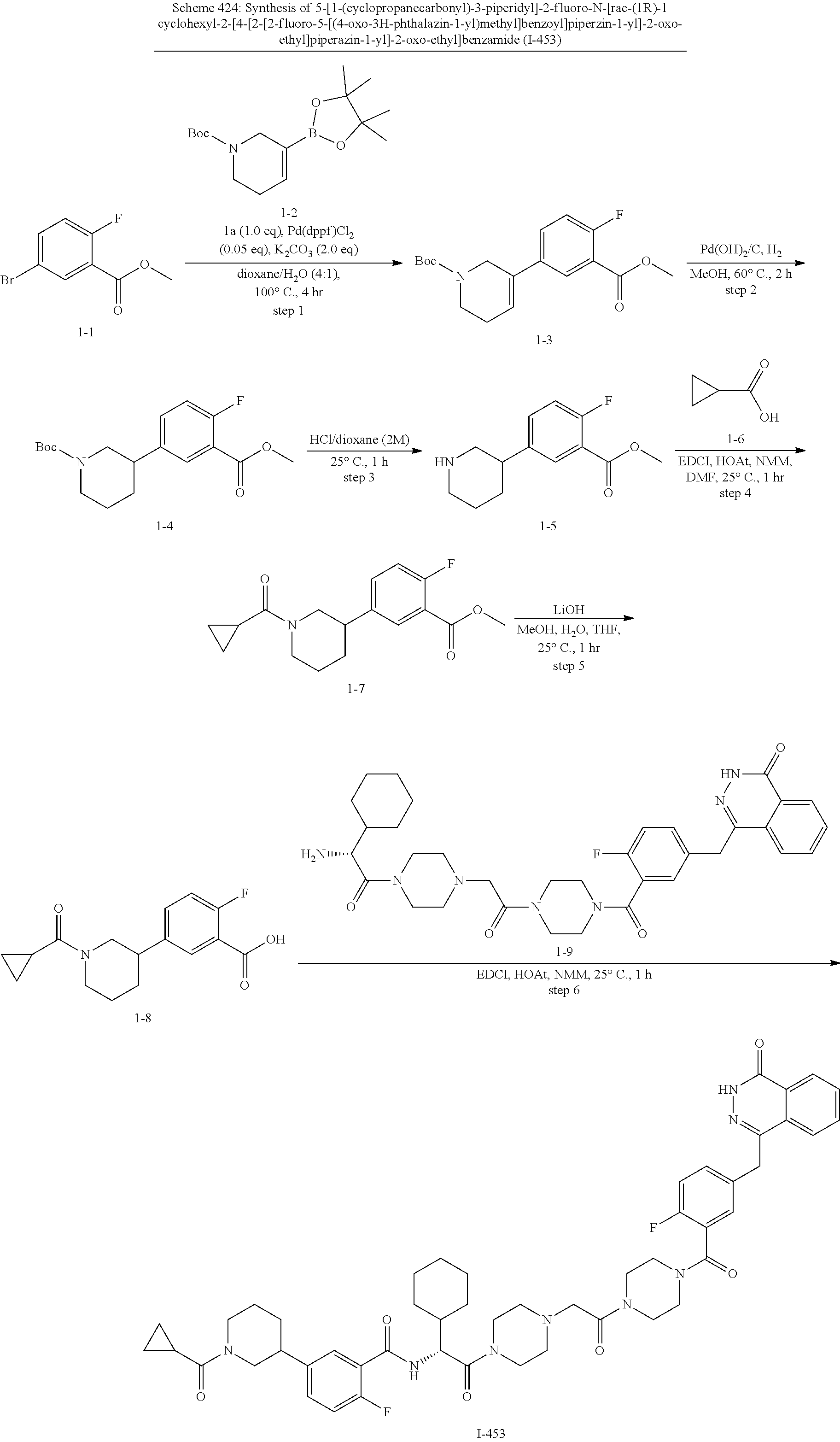

### Step 1: Synthesis of Intermediate 1-3

A mixture of Intermediate 1-1 (1 g, 4.29 mmol, 1 eq), Intermediate 1-2 (1.33 g, 4.29 mmol, 1 eq), Pd(dppf)Cl₂ (156.95 mg, 214.50 μmol, 0.05 eq) and K₂CO₃ (1.19 g, 8.58 mmol, 2 eq) in dioxane (10 mL) and H₂O (2.5 mL) was degassed and purged with N₂ 3 times, and then the mixture was stirred at 100° C. for 4 hr under N₂ atmosphere. The reaction mixture was diluted with H₂O (40 mL) and extracted with EA (40 mL*3). The combined organic layers were washed with brine (20 mL), dried over Na₂SO₄, filtered and concentrated under reduced pressure to give a residue. The residue was purified by flash silica gel chromatography (ISCO; 80 g SepaFlash Silica Flash Column, Eluent of 26% Ethyl acetate/Petroleum ether gradient, 50 mL/min). Intermediate 1-3 (1.2 g, 3.22 mmol, 75.07% yield, 90% purity) was obtained as a light yellow oil. LCMS (Method G): Rt=0.719 min, [M−99]⁺=236.2. ¹H NMR (400 MHz, CHLOROFORM-d) δ=7.97-7.90 (m, 1H), 7.56-7.46 (m, 1H), 7.16-7.06 (m, 1H), 6.24-6.18 (m, 1H), 4.25 (br s, 2H), 3.95 (s, 3H), 3.59-3.52 (m, 2H), 2.33 (br s, 2H), 1.51 (s, 9H).

### Step 2: Synthesis of Intermediate 1-4

To a solution of Pd(OH)₂/C (200 mg, 20% purity) in MeOH (20 mL) was added Intermediate 1-3 (500 mg, 1.49 mmol, 1 eq). The mixture was degassed and purged with N₂ 3 times, and then the mixture was stirred at 60° C. for 2 h under H₂ (15 psi). The mixture was filtered and the filtrate was concentrated under vacuum to give a crude product. The crude was used for the next step directly without purification. Intermediate 1-4 (460 mg, crude) was obtained as a colorless oil. LCMS (Method E): Rt=0.588 min, [M−99]⁺=238.2.

### Step 3: Synthesis of Intermediate 1-5

To a solution of Intermediate 1-4 (460 mg, 1.36 mmol, 1 eq) in DCM (5 mL) was added HCl/dioxane (2 M, 10 mL, 14.67 eq). The mixture was stirred at 25° C. for 1 h. The mixture was concentrated under vacuum to give a crude product. The crude was used for the next step without purification. Intermediate 1-5 (370 mg, crude) was obtained as a yellow solid.

### Step 4: Synthesis of Intermediate 1-7

To a solution of Intermediate 1-5 (320 mg, 1.17 mmol, 1 eq, HCl salt) in DMF (3 mL) was added HOAt (159.12 mg, 1.17 mmol, 163.53 μL, 1 eq), EDCI (448.21 mg, 2.34 mmol, 2 eq), NMM (591.22 mg, 5.85 mmol, 642.63 μL, 5 eq) and Intermediate 1-6 (100.64 mg, 1.17 mmol, 92.50 μL, 1 eq). The mixture was stirred at 25° C. for 1 h. The reaction mixture was quenched slowly by H₂O (4 mL), and extracted with EA (2 mL*3). The combined organic layer was washed with brine (2 ml*3), dried over Na₂SO₄, filtered and concentrated under reduced pressure to give a residue. The residue was purified by flash silica gel chromatography (ISCO; 12 g SepaFlashSilica Flash Column, Eluent of 0˜10% DCM/Methanol@100 mL/min). Intermediate 1-7 (230 mg, 753.25 μmol, 64.43% yield) was obtained as a yellow oil. LCMS (Method E): Rt=0.514 min, [M+H]⁺=306.2. ¹H NMR (400 MHz, CHLOROFORM-d) δ=7.81 (dd, J=2.4, 6.8 Hz, 1H), 7.40 (ddd, J=2.6, 4.6, 8.5 Hz, 1H), 7.10 (dd, J=8.7, 10.3 Hz, 1H), 4.83-4.29 (m, 2H), 3.95-3.91 (m, 3H), 2.74 (br t, J=11.0 Hz, 1H), 2.14-2.05 (m, 1H), 1.87 (br d, J=12.8 Hz, 1H), 1.80-1.61 (m, 4H), 1.03-0.94 (m, 2H), 0.79-0.72 (m, 2H).

### Step 5: Synthesis of Intermediate 1-8

To a solution of Intermediate 1-7 (230 mg, 753.25 μmol, 1 eq) in THF (2 mL), MeOH (1 mL) and H₂O (2 mL) was added LiOH (54.12 mg, 2.26 mmol, 3 eq). The mixture was stirred at 25° C. for 1 h. The mixture was concentrated under vacuum to remove THF and MeOH. Then the aqueous phase was adjusted to pH=3 and extracted with EA (2 ml*3). The combined organic layer was washed with brine (2 ml*3), dried over anhydrous Na₂SO₄, filtered and concentrated under vacuum to give crude product. The crude was used for the next step without purification. Intermediate 1-8 (170 mg, crude) was obtained as a white solid. LCMS (Method E): Rt=0.449 min, [M+H]⁺=292.1.

### Step 6: Synthesis of I-453

To a solution of Intermediate 1-8 (20 mg, 68.65 μmol, 1 eq) in DMF (1 mL) was added HOAt (9.34 mg, 68.65 μmol, 9.60 μL, 1 eq) and Intermediate 1-9 (41.29 mg, 61.79 μmol, 0.9 eq, HCl salt), EDCI (26.32 mg, 137.31 μmol, 2 eq) and NMM (34.72 mg, 343.27 μmol, 37.74 μL, 5 eq). The mixture was stirred at 25° C. for 1 h. The mixture was filtered. The filtrate was purified by Prep-HPLC (column: Waters Xbridge 150*25 mm*5 um; mobile phase: [water (NH₄HCO₃)-ACN]; gradient: 28%-58% B over 9 min). I-453 (28.09 mg, 31.00 μmol, 45.15% yield, 99.873% purity) was obtained as a white solid. LCMS (Method G): Rt=0.631 min, [M+H]⁺=905.5. SFC: Rt=4.450 min, 5.393 min ¹H NMR (400 MHz, METHANOL-d₄) δ =8.37 (d, J=7.6 Hz, 1H), 8.00-7.91 (m, 1H), 7.91-7.79 (m, 2H), 7.73-7.60 (m, 1H), 7.56-7.43 (m, 2H), 7.3-7.36 (m, 1H), 7.25-7.13 (m, 2H), 4.95 (br s, 1H), 4.61-4.49 (m, 1H), 4.44-4.33 (m, 3H), 3.87-3.66 (m, 7H), 3.65-3.53 (m, 3H), 3.31-3.20 (m, 3H), 2.81-2.44 (m, 6H), 2.11-1.56 (m, 12H), 1.47-1.01 (m, 6H), 0.91-0.74 (m, 4H).

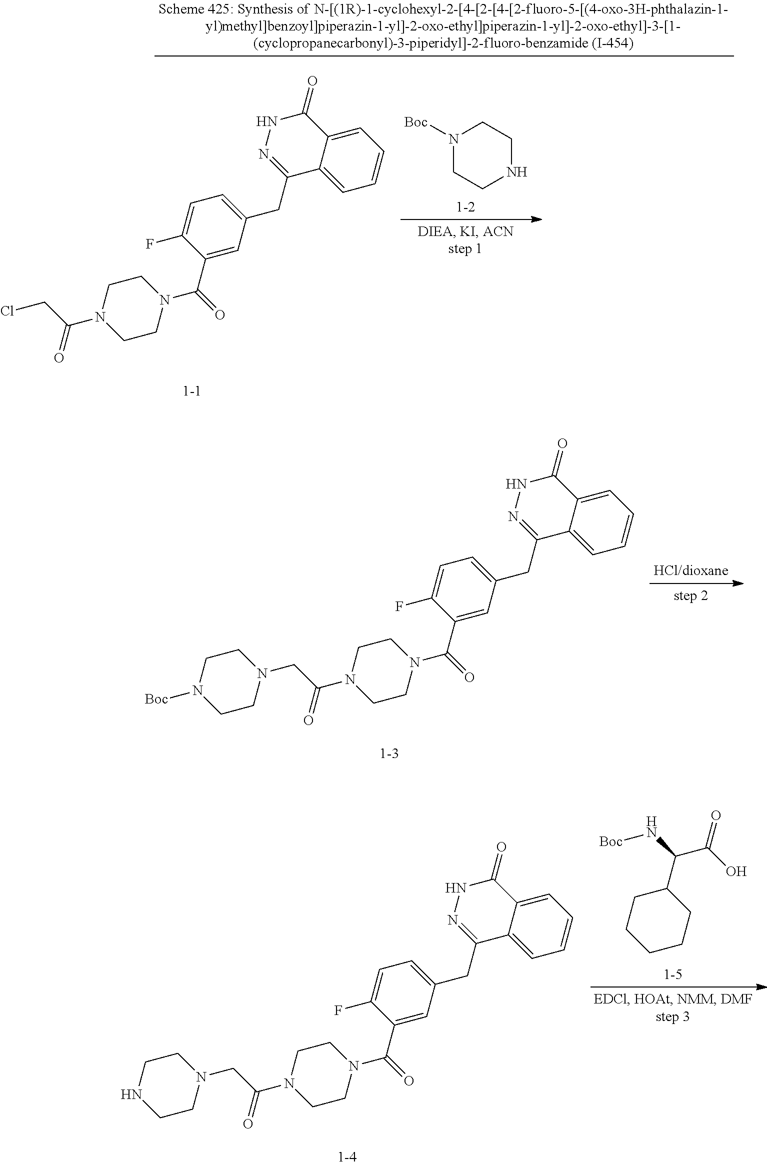 [SMILES: O=C(CCl)N1CCN(C(=O)c2cc(Cc3n[nH]c(=O)c4ccccc34)ccc2F)CC1 ; CC(C)(C)OC(=O)N1CCNCC1 ; CC(C)(C)OC(=O)N1CCN(CC(=O)N2CCN(C(=O)c3cc(Cc4n[nH]c(=O)c5ccccc45)ccc3F)CC2)CC1 ; O=C(CN1CCNCC1)N1CCN(C(=O)c2cc(Cc3n[nH]c(=O)c4ccccc34)ccc2F)CC1 ; CC(C)(C)OC(=O)N[C@@H](C(=O)O)C1CCCCC1]

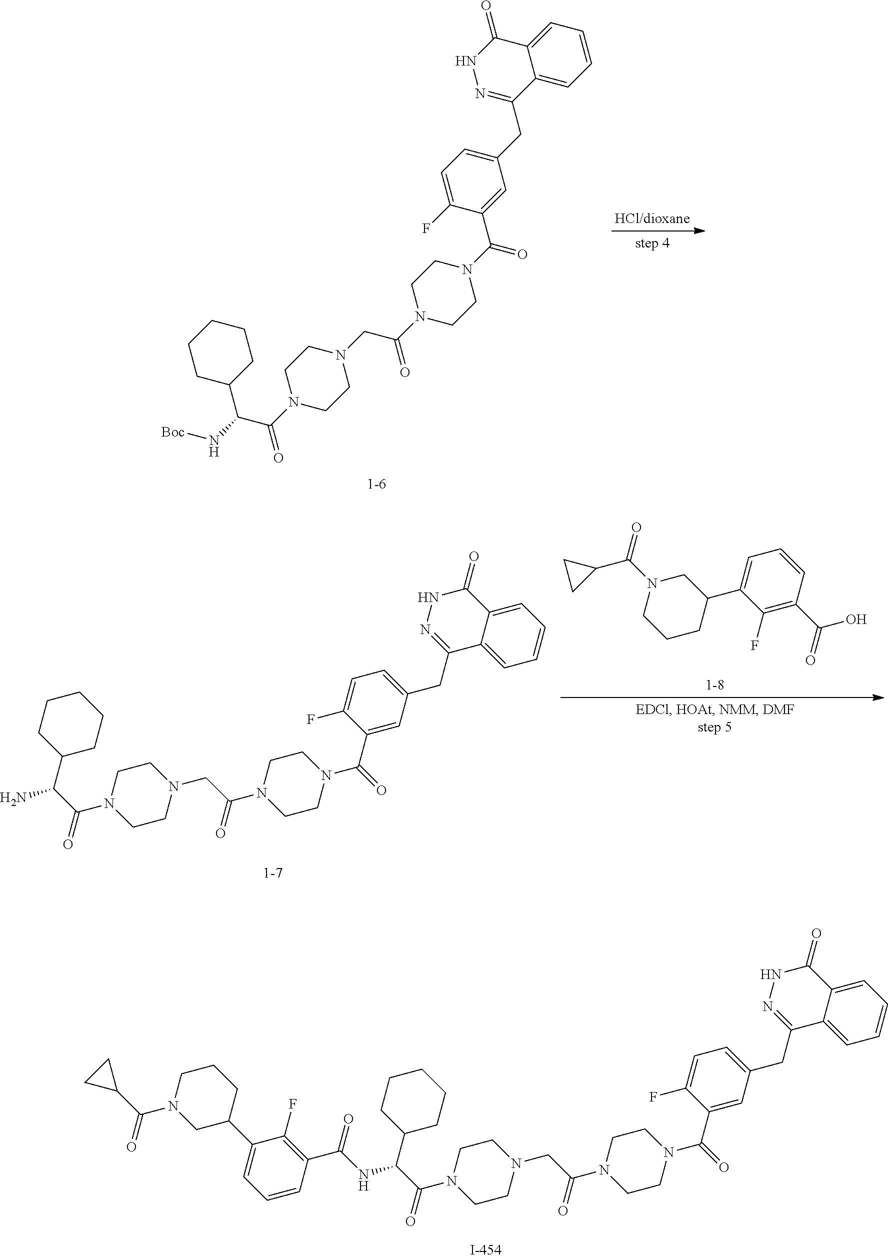

### Step 1: Synthesis of Intermediate 1-3

To a solution of Intermediate 1-1 (1 g, 2.26 mmol, 1 eq) in ACN (10 mL) was added Intermediate 1-2 (630.83 mg, 3.39 mmol, 1.5 eq), DIEA (1.17 g, 9.03 mmol, 1.57 mL, 4 eq) and KI (1.50 g, 9.03 mmol, 4 eq). The mixture was stirred at 40° C. for 2 hr. The reaction mixture was diluted with water (60 mL) and extracted with EA (20 mL*3). The combined organic layers were dried over Na₂SO₄, filtered and concentrated under reduced pressure to give a residue. The residue was combined with another lot of material (100 mg of Intermediate 1-1 starting material) for purification. The residue was purified by flash silica gel chromatography (ISCO; 40 g SepaFlash Silica Flash Column, Eluent of 0˜100% ethyl acetate/MeOH @60 mL/min). Intermediate 1-3 (1 g, 1.59 mmol, 70.24% yield, 94% purity) was obtained as a yellow solid. LCMS (Method E): Rt=0.421 min, [M+H]⁺=595.3. ¹H NMR (400 MHz, CHLOROFORM-d) δ=10.13 (d, J=5.6 Hz, 1H), 8.50-8.42 (m, 1H), 7.82-7.76 (m, 2H), 7.75-7.68 (m, 1H), 7.39-7.30 (m, 2H), 7.08-7.06 (m, 1H), 4.29 (s, 2H), 3.73-3.65 (m, 2H), 3.62-3.56 (m, 2H), 3.51-3.43 (m, 4H), 3.41 (br s, 2H), 3.35-3.26 (m, 2H), 3.26-3.18 (m, 2H), 2.47-2.44 (m, 4H), 1.46 (s, 9H). ¹⁹F NMR (377 MHz, CHLOROFORM-d).

### Step 2: Synthesis of Intermediate 1-4

A solution of Intermediate 1-3 (500 mg, 843.65 μmol, 1 eq) in HCl/dioxane (2 M, 10 mL, 23.71 eq) was stirred at 25° C. for 0.5 hr. The mixture was concentrated under vacuum. The residue was used in the next step without purification. Intermediate 1-4 (440 mg, crude, HCl salt) was obtained as a light yellow solid. LCMS (Method E): Rt=0.349 min, [M+H]⁺=493.3.

### Step 3: Synthesis of Intermediate 1-6

To a solution of Intermediate 1-4 (440 mg, 831.75 μmol, 1 eq, HCl salt) in DMF (4.5 mL) was added Intermediate 1-5 (214.03 mg, 831.75 μmol, 1 eq), EDCI (318.89 mg, 1.66 mmol, 2 eq), NMM (420.65 mg, 4.16 mmol, 457.22 μL, 5 eq) and HOAt (113.21 mg, 831.75 μmol, 116.35 μL, 1 eq). The mixture was stirred at 25° C. for 2 hr. The reaction mixture was triturated with water (15 ml), filtered and the filter cake was washed with water (10 mL*2), and the filter cake was dried under vacuum. Intermediate 1-6 (520 mg, 710.52 μmol, 85.43% yield) was obtained as a yellow oil. LCMS (Method G): Rt=0.603 min, [M+H]⁺=732.5. SFC: Rt=1.910 min.

### Step 4: Synthesis of Intermediate 1-7

To a solution of Intermediate 1-6 (50 mg, 68.32 μmol, 1 eq) in DCM (0.5 mL) was added HCl/dioxane (2 M, 1 mL, 29.27 eq). The mixture was stirred at 25° C. for 0.5 hr. The mixture was concentrated under vacuum. The residue was used in the next step without purification. Intermediate 1-7 (45 mg, crude, HCl salt) was obtained as a light yellow solid. LCMS (Method E): Rt=0.384 min, [M+H]+=632.4.

### Step 5: Synthesis of I-454

To a solution of Intermediate 1-7 (45 mg, 67.35 μmol, 1 eq, HCl salt) in DMF (0.5 mL) was added Intermediate 1-8 (19.62 mg, 67.35 μmol, 1 eq), EDCI (12.91 mg, 67.35 μmol, 1 eq), NMM (6.81 mg, 67.35 μmol, 7.40 μL, 1 eq) and HOAt (9.17 mg, 67.35 μmol, 9.42 μL, 1 eq). The mixture was stirred at 25° C. for 2 hr. The mixture was diluted with water (10 mL), and extracted with EtOAc (10 mL*3). The organic layer was concentrated under vacuum. The residue was purified by Prep-HPLC (column: Phenomenex luna C18 150*25 mm*10 um; mobile phase: [water (FA)-ACN]; gradient: 20%-50% B over 9 min) and dried by lyophilization. I-454 (21.89 mg, 23.90 μmol, 35.49% yield, 98.82% purity) was obtained as a white solid. LCMS (Method E): Rt=0.479 min, [M+H]⁺=905.7. SFC: Rt=3.774 min, 4.912 min. ¹H NMR (400 MHz, METHANOL-d₄) δ=8.37 (d, J=7.6 Hz, 1H), 7.98-7.92 (m, 1H), 7.90-7.78 (m, 2H), 7.62-7.45 (m, 3H), 7.41-7.35 (m, 1H), 7.31-7.21 (m, 1H), 7.19-7.15 (m, 1H), 4.99-4.87 (m, 2H), 4.63-4.53 (m, 1H), 4.46-4.35 (m, 3H), 3.90-3.63 (m, 7H), 3.61-3.47 (m, 3H), 3.33 (br s, 2H), 3.28-2.99 (m, 3H), 2.89-2.68 (m, 1H), 2.67-2.44 (m, 4H), 2.07-1.58 (m, 11H), 1.36-1.07 (m, 5H), 0.95-0.72 (m, 4H).

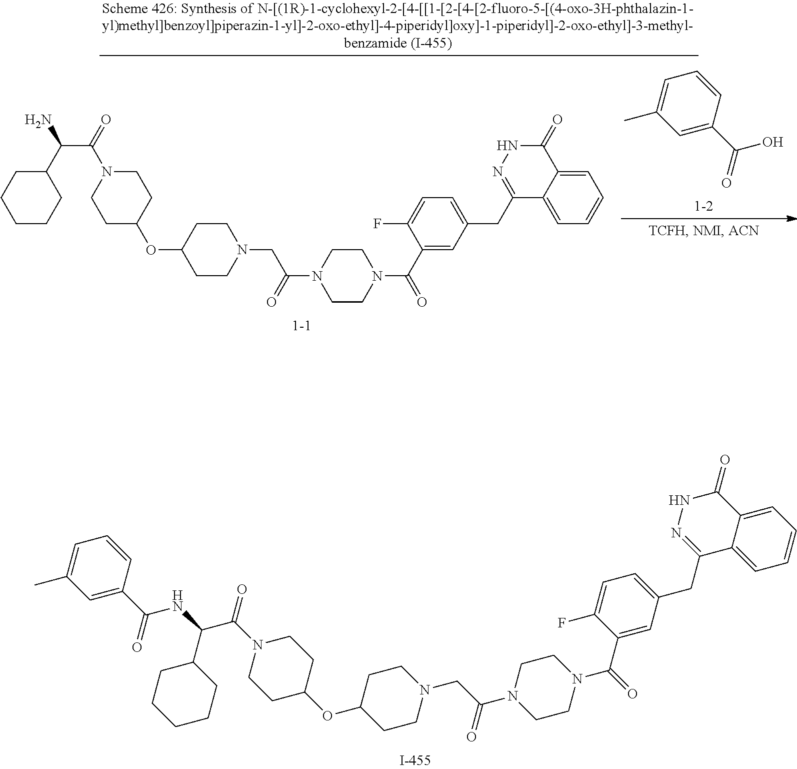

To a solution of Intermediate 1-1 (50 mg, 65.25 μmol, 1 eq, HCl salt) in MeCN (0.5 mL) was added Intermediate 1-2 (8.88 mg, 65.25 μmol, 8.43 μL, 1 eq), TCFH (21.97 mg, 78.29 μmol, 1.2 eq) and NMI (18.75 mg, 228.36 μmol, 18.20 μL, 3.5 eq). The mixture was stirred at 25° C. for 1 hr. The mixture was diluted with water (10 mL), and extracted with EtOAc (10 mL*3). The organic layer was concentrated under vacuum. The residue was purified by Prep-HPLC (column: Waters Xbridge 150*25 mm*5 um; mobile phase: [water (NH₄HCO₃)-ACN]; gradient: 32%-62% B over 15 min) and dried by lyophilization. I-455 (17.18 mg, 20.22 μmol, 95.27% yield, 99.82% purity) was obtained as a white solid. LCMS (Method G): Rt=0.652 min, [M+H]⁺=848.6. SFC: Rt=1.905 min, 2.735 min, ee %=90.604%. ¹H NMR (400 MHz, METHANOL-d₄) δ=8.36 (br d, J=8.0 Hz, 1H), 7.94 (br d, J=8.0 Hz, 1H), 7.90-7.80 (m, 2H), 7.65 (s, 1H), 7.61 (br d, J=8.0 Hz, 1H), 7.48 (br s, 1H), 7.41-7.31 (m, 3H), 7.19-7.12 (m, 1H), 4.93 (br d, J=8.0 Hz, 1H), 4.38 (s, 2H), 4.09-3.99 (m, 1H), 3.97-3.84 (m, 1H), 3.82-3.71 (m, 4H), 3.66 (br s, 1H), 3.62-3.42 (m, 5H), 3.30 (br s, 1H), 3.26-3.17 (m, 2H), 2.83-2.70 (m, 2H), 2.39 (s, 3H), 2.32-2.19 (m, 2H), 1.99-1.72 (m, 9H), 1.68 (br d, J=12.0 Hz, 2H), 1.64-1.44 (m, 4H), 1.35-1.21 (m, 3H), 1.16-1.04 (m, 2H). ¹⁹F NMR (377 MHz, METHANOL-d₄) δ=−120.731.

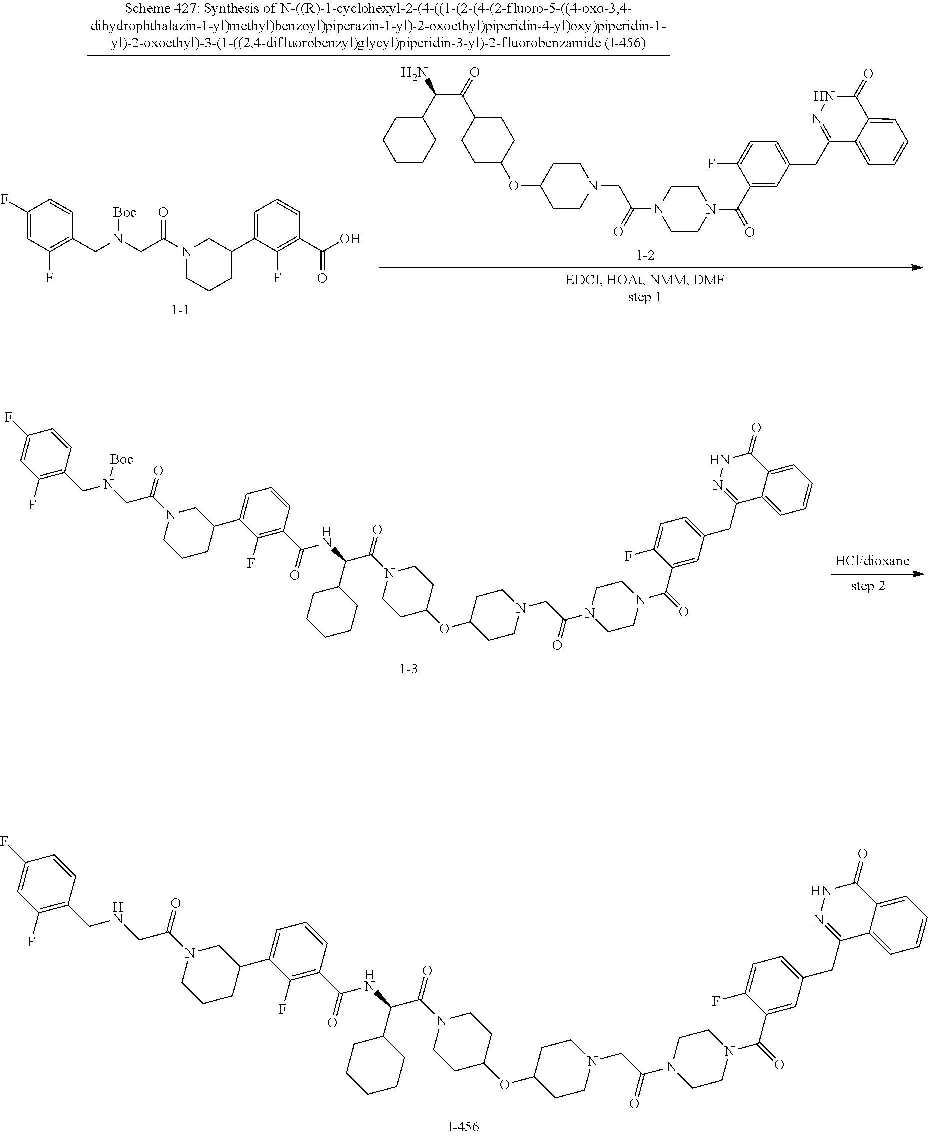 [SMILES: CC(C)(C)OC(=O)N(CC(=O)N1CCCC(c2cccc(C(=O)O)c2F)C1)Cc1ccc(F)cc1F ; N[C@@H](C(=O)C1CCC(OC2CCN(CC(=O)N3CCN(C(=O)c4cc(Cc5n[nH]c(=O)c6ccccc56)ccc4F)CC3)CC2)CC1)C1CCCCC1 ; CC(C)(C)OC(=O)N(CC(=O)N1CCCC(c2cccc(C(=O)N[C@@H](C(=O)N3CCC(OC4CCN(CC(=O)N5CCN(C(=O)c6cc(Cc7n[nH]c(=O)c8ccccc78)ccc6F)CC5)CC4)CC3)C3CCCCC3)c2F)C1)Cc1ccc(F)cc1F ; O=C(N[C@@H](C(=O)N1CCC(OC2CCN(CC(=O)N3CCN(C(=O)c4cc(Cc5n[nH]c(=O)c6ccccc56)ccc4F)CC3)CC2)CC1)C1CCCCC1)c1cccc(C2CCCN(C(=O)CNCc3ccc(F)cc3F)C2)c1F]

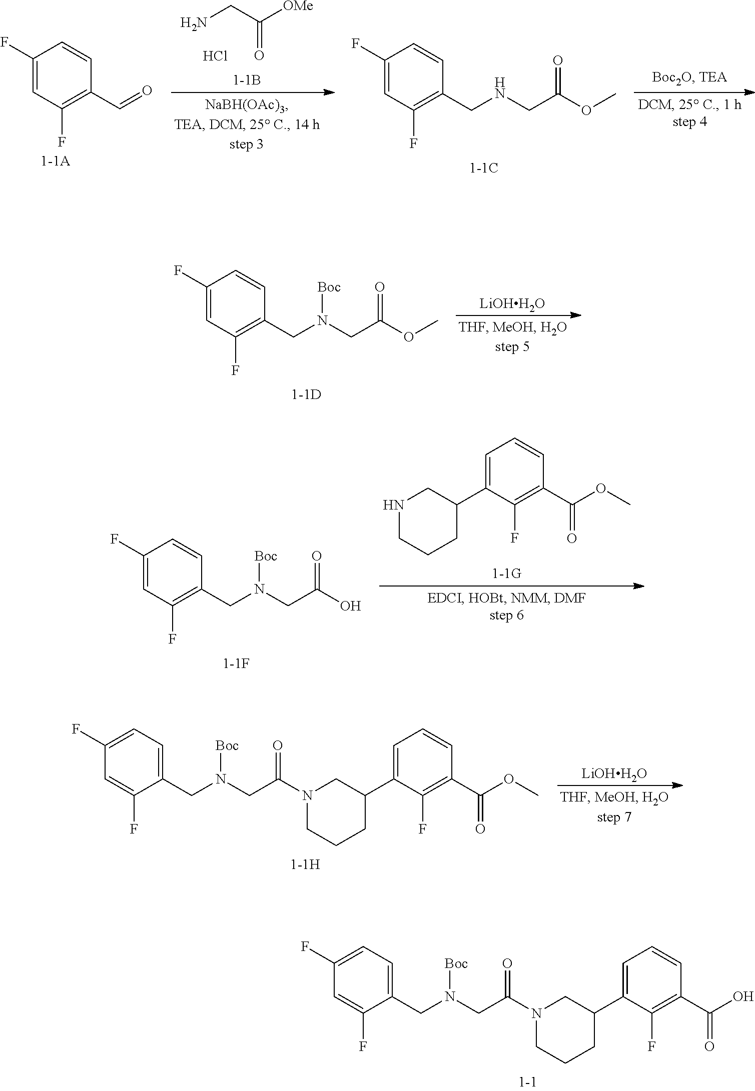

### Step 1: Synthesis of Intermediate 1-3

To a solution of Intermediate 1-1 (200.93 mg, 396.69 μmol, 1 eq) in DMF (3 mL) was added Intermediate 1-2 (304 mg, 396.69 μmol, 1 eq, HCl salt), EDCI (152.09 mg, 793.38 μmol, 2 eq), NMM (200.62 mg, 1.98 mmol, 218.07 μL, 5 eq) and HOAt (53.99 mg, 396.69 μmol, 55.49 μL, 1 eq). The mixture was stirred at 25° C. for 1 hr. The reaction mixture was triturated with water (30 ml), filtered and the filter cake was washed with water (10 mL*2), and the filter cake was dried under vacuum. Intermediate 1-3 (370 mg, 303.68 μmol, 76.55% yield) was obtained as a yellow solid. LCMS (Method G): Rt=0.768 min, [M+H]⁺=1218.8.

### Step 2: Synthesis of I-456

To a solution of Intermediate 1-3 (370 mg, 303.68 μmol, 1 eq) in DCM (3 mL) was added HCl/dioxane (4 M, 3 mL, 39.51 eq). The mixture was stirred at 25° C. for 0.5 hr. The mixture was concentrated under vacuum. The residue was purified by Pre-HPLC (column: Waters Xbridge 150*25 mm*5 um; mobile phase: [water (NH₄HCO₃)-ACN]; gradient: 38%-68% B over 15 min) and dried by lyophilized. I-456 (169.55 mg, 146.19 μmol, 48.14% yield, 96.42% purity) was obtained as a white solid. LCMS (Method E): Rt=1.072 min, 1.105 min, [M+H]⁺=1118.6. HPLC: Rt=3.198 min, 3.290 min. SFC: Rt=3.779 min, 4.563 min. ¹H NMR (400 MHz, METHANOL-d₄) δ=8.37 (br d, J=7.6 Hz, 1H), 7.98-7.92 (m, 1H), 7.90-7.80 (m, 2H), 7.61-7.54 (m, 1H), 7.51-7.42 (m, 3H), 7.37 (br d, J=4.4 Hz, 1H), 7.29-7.22 (m, 1H), 7.19-7.15 (m, 1H), 6.98-6.90 (m, 2H), 5.01-4.93 (m, 1H), 4.60-4.54 (m, 1H), 4.38 (s, 2H), 4.07-3.98 (m, 1H), 3.95-3.85 (m, 2H), 3.85-3.69 (m, 7H), 3.69-3.63 (m, 1H), 3.60-3.44 (m, 7H), 3.27 (br d, J=4.4 Hz, 2H), 3.21 (br s, 1H), 3.15-3.03 (m, 2H), 2.84-2.68 (m, 3H), 2.34-2.22 (m, 2H), 2.04-1.99 (m, 1H), 1.94-1.76 (m, 10H), 1.71-1.50 (m, 7H), 1.33-1.21 (m, 3H), 1.20-1.11 (m, 2H). ¹⁹F NMR (377 MHz, METHANOL-d₄) δ=−113.336, −116.232, −120.792, −121.733.

### Step 3: Synthesis of Intermediate 1-1C

To a solution of Intermediate 1-1A (20 g, 140.74 mmol, 1 eq) and Intermediate 1-1B (35.34 g, 281.49 mmol, 2 eq, HCl salt) in DCM (200 mL) was added TEA (28.48 g, 281.49 mmol, 39.18 mL, 2 eq). The mixture was stirred at 25° C. for 2 hr. Then the NaBH(OAc)₃ (44.74 g, 211.11 mmol, 1.5 eq) was added and the mixture was stirred at 25° C. for 12 hr. The reaction mixture was quenched by H₂O (400 mL), and extracted with DCM (200 mL*3). The combined organic layers were washed with brine (200 mL*3), dried over anhydrous Na₂SO₄, filtered and concentrated under reduced pressure to give a crude product. The crude was used for the next step without purification. Intermediate 1-1C (31 g, crude) was obtained as a yellow oil. LCMS (Method E): Retention time: 0.218 min, [M+H]⁺=216.0.

### Step 4: Synthesis of Intermediate 1-1D

To a solution of Intermediate 1-1C (31 g, 144.05 mmol, 1 eq) in DCM (300 mL) was added TEA (17.49 g, 172.87 mmol, 24.06 mL, 1.2 eq) and (Boc)₂O (37.73 g, 172.87 mmol, 39.71 mL, 1.2 eq). The mixture was stirred at 25° C. for 1 h. The reaction mixture was quenched by H₂O (300 mL), and extracted with DCM (200 mL*3). The combined organic layers were washed with brine (300 mL*3), dried over anhydrous Na₂SO₄, filtered and concentrated under reduced pressure to give a residue. The residue was purified by flash silica gel chromatography (ISCO; 330 g SepaFlash Silica Flash Column, Eluent of 0˜10% Ethyl acetate/Petroleum ether gradient @100 mL/min). Intermediate 1-1D (30.5 g, 96.73 mmol, 67.15% yield) was obtained as a yellow oil. LCMS (Method E): Retention time: 0.591 min, [M-Boc+H]⁺=216.1. ¹H NMR (400 MHz, CHLOROFORM-d) δ=7.41-7.12 (m, 1H), 6.84-6.65 (m, 2H), 4.43 (br d, J=15.1 Hz, 2H), 3.94-3.76 (m, 2H), 3.64 (d, J=3.0 Hz, 3H), 1.38 (d, J=8.5 Hz, 9H). ¹⁹F NMR (377 MHz, CHLOROFORM-d) δ=−111.005, −111.029, −113.830, −114.712.

### Step 5: Synthesis of Intermediate 1-1F

To a solution of Intermediate 1-1D (30.5 g, 96.73 mmol, 1 eq) in THF (100 mL), MeOH (100 mL) and H₂O (100 mL) was added LiOH·H₂O (8.12 g, 193.46 mmol, 2 eq). The mixture was stirred at 25° C. for 1 h. The mixture was concentrated under vacuum to remove THF and MeOH. Then the aqueous phase was adjusted to pH=5 and extracted with EA (100 ml*3). The combined organic layers were washed with brine (100 ml*3), dried over anhydrous Na₂SO₄, filtered and concentrated under vacuum to a crude product. The crude product was used for the next step directly without purification. Intermediate 1-2 (27 g, crude) was obtained as a white solid. LCMS (Method G): Retention time: 0.316 min, [M-Boc+H]⁺=202.1. ¹H NMR (400 MHz, DMSO-d₆) δ=7.44-7.25 (m, 1H), 7.16 (br t, J=9.9 Hz, 1H), 7.09-6.97 (m, 1H), 4.41 (br d, J=3.4 Hz, 2H), 3.72-3.47 (m, 2H), 1.34 (d, J=18.4 Hz, 9H). ¹⁹F NMR (377 MHz, DMSO-d₆) δ=−112.274, −114.223, −114.503.

### Step 6: Synthesis of Intermediate 1-1H

A mixture of Intermediate 1-3 (0.4 g, 1.46 mmol, 1 eq, HCl), Intermediate 1-2 (440.27 mg, 1.46 mmol, 1 eq) and HOAt (198.90 mg, 1.46 mmol, 204.42 μL, 1 eq) in DMF (4 mL) was added EDCI (560.26 mg, 2.92 mmol, 2 eq) and NMM (739.05 mg, 7.31 mmol, 803.31 μL, 5 eq). The mixture was stirred at 25° C. for 2 hrs. The mixture was diluted with water (10 mL), and extracted with EtOAc (10 mL*3). The organic layer was dried with anhydrous Na₂SO₄, filtered and concentrated under vacuum. The crude product was purified by reversed-phase HPLC (0.1% NH₃·H₂O condition) and concentrated under vacuum. Intermediate 1-1F (620 mg, 1.19 mmol, 81.51% yield) was obtained as a yellow oil. LCMS (Method G): Retention time: 0.752 min, [M-Boc+H]⁺=421.3.

### Step 7: Synthesis of Intermediate 1-1

To a solution of Intermediate 1-1H (620 mg, 1.11 mmol, 1 eq) in THF (3 mL), H₂O (1.5 mL) and MeOH (1.5 mL) was added LiOH·H₂O (149.93 mg, 3.57 mmol, 3 eq). The mixture was stirred at 25° C. for 2 hr. The mixture was adjusted to pH=5 with 2N HCl and concentrated under vacuum. The crude product was purified by reversed-phase HPLC (0.1% FA condition) and concentrated under vacuum to remove MeCN and dried by lyophilization. Intermediate 1-1 (0.6 g, crude) was obtained as a colorless oil. LCMS (Method G): Retention time: 0.419 min, [M-Boc+H]⁺=407.2.

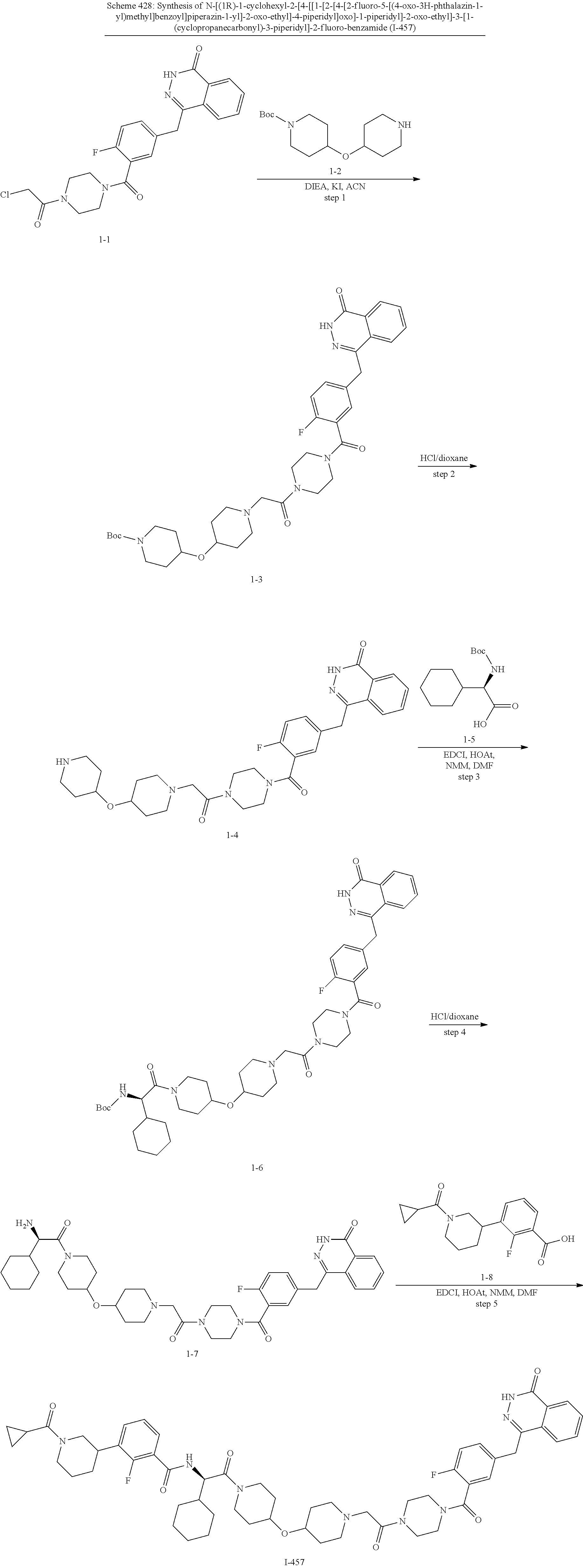

### Step 1: Synthesis of Intermediate 1-3

To a solution of Intermediate 1-1 (800 mg, 1.81 mmol, 1 eq) in DMF (8 mL) was added Intermediate 1-2 (770.59 mg, 2.71 mmol, 1.5 eq), DIEA (933.85 mg, 7.23 mmol, 1.26 mL, 4 eq) and KI (1.20 g, 7.23 mmol, 4 eq). The mixture was stirred at 25° C. for 1 hr. The crude product was purified by reversed-phase HPLC (0.1% NH₃H₂O condition) and concentrated under vacuum to remove MeCN and dried by lyophilization. Intermediate 1-3 (950 mg, 1.38 mmol, 76.13% yield) was obtained as a white solid. LCMS (Method G): Rt=0.609 min, [M+H]⁺=691.4.

### Step 2: Synthesis of Intermediate 1-4

To a solution of Intermediate 1-3 (950 mg, 1.38 mmol, 1 eq) in DCM (9 mL) was added HCl/dioxane (2 M, 18 mL, 26.18 eq). The mixture was stirred at 25° C. for 0.5 hr. The mixture was concentrated under vacuum. The residue was used in the next step without purification. Intermediate 1-4 (850 mg, 1.36 mmol, 98.56% yield, HCl salt) was obtained as a light yellow solid. LCMS (Method E): Rt=0.352 min, [M+H]⁺=591.3.

### Step 3: Synthesis of Intermediate 1-6

To a solution of Intermediate 1-4 (800 mg, 1.28 mmol, 1 eq, HCl salt) in DMF (8 mL) was added Intermediate 1-5 (328.25 mg, 1.28 mmol, 1 eq), EDCI (489.07 mg, 2.55 mmol, 2 eq), NMM (645.12 mg, 6.38 mmol, 701.22 μL, 5 eq) and HOAt (173.63 mg, 1.28 mmol, 178.44 μL, 1 eq). The mixture was stirred at 25° C. for 2 hr. The mixture was diluted with water (50 mL), extracted with EtOAc (50 mL*3) and the organic layers were concentrated under vacuum. The crude product was purified by reversed-phase HPLC (0.1% NH₃H₂O condition) and concentrated under vacuum to remove MeCN and dried by lyophilization. Intermediate 1-6 (770 mg, 927.71 μmol, 72.73% yield) was obtained as a yellow oil. LCMS (Method G): Rt=0.667 min, [M+H]⁺=830.5.

### Step 4: Synthesis of Intermediate 1-7

To a solution of Intermediate 1-6 (770 mg, 927.71 μmol, 1 eq) in DCM (5 mL) was added HCl/dioxane (2 M, 10 mL, 21.56 eq). The mixture was stirred at 25° C. for 0.5 hr. The mixture was concentrated under vacuum. The residue was used in the next step without purification. Intermediate 1-7 (700 mg, crude, HCl salt) was obtained as a light yellow solid. LCMS (Method E): Rt=0.401 min, [M+H]+=730.4.

### Step 5: Synthesis of I-457

To a solution of Intermediate 1-7 (700 mg, 913.43 μmol, 1 eq, HCl salt) in DMF (7 mL) was added Intermediate 1-8 (266.10 mg, 913.43 μmol, 1 eq), EDCI (350.21 mg, 1.83 mmol, 2 eq), NMM (461.95 mg, 4.57 mmol, 502.12 μL, 5 eq) and HOAt (124.33 mg, 913.43 μmol, 127.78 μL, 1 eq). The mixture was stirred at 25° C. for 2 hr. The mixture was diluted with water (10 mL), and extracted with EtOAc (30 mL*3). The organic layer was concentrated under vacuum. The residue was purified by Prep-HPLC (column: Waters Xbridge Prep OBD C18 150*40 mm*10 um; mobile phase: [water (NH₄HCO₃)-ACN]; gradient: 33%-63% B over 28 min) and dried by lyophilization. The residue was repurified by Prep-HPLC (column: Waters Xbridge Prep OBD C18 150*40 mm*10 um; mobile phase: [water (NH₄HCO₃)-ACN]; gradient: 33%-63% B over 15 min) and dried by lyophilization. I-457 (315.92 mg, 314.92 μmol, 34.48% yield, 100% purity) was obtained as an off-white solid. LCMS (Method G): Rt=0.666 min, [M+H]⁺=1003.6. SFC: Rt=2.553 min, 3.454 min. Special LCMS: Rt=13.798 min, 14.186 min, [M+H]⁺=1003.8. ¹H NMR (400 MHz, DMSO-d₆) δ=12.59 (s, 1H), 8.44-8.32 (m, 1H), 8.26 (d, J=7.6 Hz, 1H), 8.00-7.93 (m, 1H), 7.92-7.79 (m, 2H), 7.54-7.34 (m, 4H), 7.26-7.20 (m, 2H), 4.84-4.70 (m, 1H), 4.43-4.40 (m, 1H), 4.38-4.24 (m, 3H), 4.02-3.80 (m, 2H), 3.71-3.54 (m, 4H), 3.53-3.34 (m, 5H), 3.14 (br s, 4H), 3.10-2.97 (m, 2H), 2.93-2.57 (m, 4H), 2.17-2.05 (m, 2H), 2.01-1.88 (m, 2H), 1.85-1.65 (m, 10H), 1.59 (br d, J=10.8 Hz, 2H), 1.50-1.23 (m, 5H), 1.19-0.97 (m, 5H), 0.79-0.63 (m, 4H). ¹⁹F NMR (377 MHz, DMSO-d₆) δ=−119.777, −120.260.

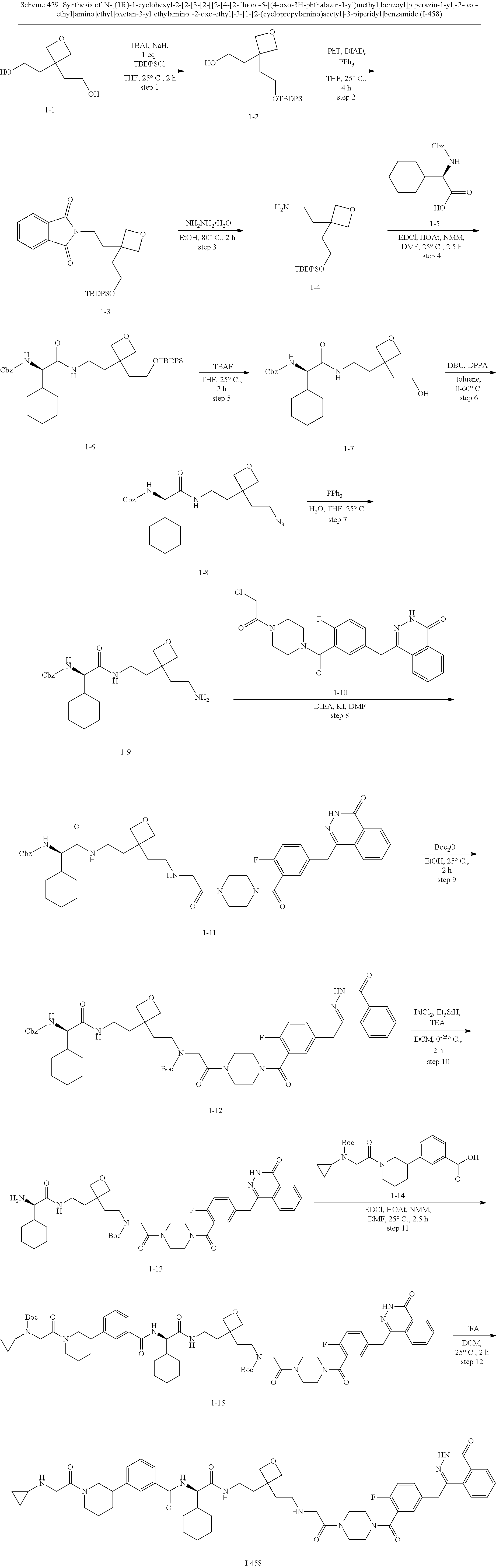

### Step 1: Synthesis of Intermediate 1-2

To a solution of Intermediate 1-1 (3 g, 20.52 mmol, 1 eq) in THF (20 mL) was added NaH (1.64 g, 41.04 mmol, 60% purity, 2 eq) at 0° C. Then TBAI (1.52 g, 4.10 mmol, 0.2 eq) and TBDPSCl (5.64 g, 20.52 mmol, 5.25 mL, 1 eq) was added. The mixture was stirred at 25° C. for 2 hr. The mixture was poured into 100 mL of aq·NH₄Cl, extracted with EA (100 mL*2). The combined organic phase was dried over anhydrous Na₂SO₄, filtered and concentrated in vacuum. The residue was purified by flash silica gel chromatography (ISCO; 330 g SepaFlash Silica Flash Column, Eluent of 0˜50% Ethyl acetate/Petroleum ether gradient @100 mL/min). Intermediate 1-2 (5 g, 13.00 mmol, 63.35% yield) was obtained as yellow oil. LCMS (Method E): Rt=0.661 min, [M+Na]⁺=407.2. ¹H NMR (400 MHz, CHLOROFORM-d) δ=7.67 (dd, J=1.5, 7.8 Hz, 4H), 7.48-7.37 (m, 6H), 4.54-4.49 (m, 2H), 4.49-4.45 (m, 2H), 3.74-3.65 (m, 4H), 2.01 (td, J=6.6, 11.2 Hz, 4H), 1.05 (s, 9H).

### Step 2: Synthesis of Intermediate 1-3

To a solution of Intermediate 1-2 (5 g, 13.00 mmol, 1 eq) and isoindoline-1,3-dione (3.83 g, 26.00 mmol, 2 eq) in THF (100 mL) was added PPh₃ (6.82 g, 26.00 mmol, 2 eq) and DIAD (5.26 g, 26.00 mmol, 5.04 mL, 2 eq). The mixture was stirred at 25° C. for 4 hr. The mixture was concentrated in vacuum. The residue was purified by flash silica gel chromatography (ISCO; 120 g SepaFlash Silica Flash Column, Eluent of 0˜50% Ethyl acetate/Petroleum ether gradient @100 mL/min). Intermediate 1-3 (5 g, 9.73 mmol, 74.87% yield) was obtained as a white solid. LCMS (Method E): Rt=0.701 min, [M+H]⁺=514.2. ¹H NMR (400 MHz, CHLOROFORM-d) δ=7.87 (dd, J=3.0, 5.5 Hz, 2H), 7.75-7.71 (m, 2H), 7.70-7.66 (m, 4H), 7.44-7.36 (m, 6H), 4.55 (d, J=6.0 Hz, 2H), 4.48 (d, J=6.0 Hz, 2H), 3.81 (t, J=6.3 Hz, 2H), 3.73-3.60 (m, 2H), 2.13-2.06 (m, 4H), 1.04 (s, 9H).

### Step 3: Synthesis of Intermediate 1-4

To a solution of Intermediate 1-3 (5 g, 9.73 mmol, 1 eq) in EtOH (100 mL) was added NH₂NH₂·H₂O (4.71 g, 94.09 mmol, 4.56 mL, 9.67 eq). The mixture was stirred at 80° C. for 2 hr. The mixture was filtered through a pad of celite and the filtrate was concentrated in vacuum. The crude product was purified by reversed-phase HPLC (0.1% FA condition). Intermediate 1-4 (4.8 g, crude) was obtained as a yellow gum. LCMS (Method E): Rt=0.487 min, [M+H]⁺=384.3. ¹H NMR (400 MHz, CHLOROFORM-d) δ=8.45 (s, 2H), 7.74-7.63 (m, 8H), 7.53-7.39 (m, 12H), 4.55 (d, J=6.2 Hz, 2H), 4.39 (d, J=6.2 Hz, 2H), 3.77-3.70 (m, 4H), 3.69-3.64 (m, 1H), 3.57-3.53 (m, 1H), 3.45 (d, J=11.7 Hz, 1H), 3.43-3.35 (m, 1H), 3.32-3.22 (m, 1H), 2.94 (d, J=11.7 Hz, 1H), 2.89-2.80 (m, 2H), 2.14-2.01 (m, 3H), 1.92 (t, J=6.4 Hz, 2H), 1.89-1.83 (m, 1H), 1.83-1.78 (m, 1H), 1.77-1.69 (m, 1H), 1.06 (s, 9H).

### Step 4: Synthesis of Intermediate 1-6

To a solution of Intermediate 1-5 (3.65 g, 12.51 mmol, 1 eq) in DMF (90 mL) was added HOAt (3.41 g, 25.03 mmol, 3.50 mL, 2 eq) and EDCI (4.80 g, 25.03 mmol, 2 eq). The mixture was stirred at 25° C. for 30 min. Then Intermediate 1-4 (4.8 g, 12.51 mmol, 1 eq) and NMM (3.80 g, 37.54 mmol, 4.13 mL, 3 eq) were added. The mixture was stirred at 25° C. for 2 hr. The mixture was poured into water (100 mL) and extracted with EA (100 mL*2). The combined organic phase was dried over anhydrous Na₂SO₄, filtered and concentrated in vacuum. The residue was purified by flash silica gel chromatography (ISCO; 120 g SepaFlash Silica Flash Column, Eluent of 0-100% Ethyl acetate/Petroleum ether gradient @100 mL/min). Intermediate 1-6 (2.2 g, 3.35 mmol, 26.76% yield) was obtained as a yellow oil. LCMS (Method E): Rt=0.714 min, [M+H]⁺=657.4. SFC: Rt=1.906 min. ¹H NMR (400 MHz, CHLOROFORM-d) δ=7.64-7.50 (m, 4H), 7.39-7.23 (m, 11H), 5.73 (br t, J=5.3 Hz, 1H), 5.21 (br d, J=3.3 Hz, 1H), 5.03 (s, 2H), 4.42 (d, J=6.0 Hz, 2H), 4.31 (br d, J=5.9 Hz, 2H), 3.80 (dd, J=6.7, 8.7 Hz, 1H), 3.64 (t, J=6.2 Hz, 2H), 3.10 (br d, J=7.6 Hz, 2H), 1.87 (br t, J=6.2 Hz, 2H), 1.81 (br d, J=3.1 Hz, 2H), 1.73-1.54 (m, 7H), 1.19-0.98 (m, 4H), 0.96 (s, 9H).

### Step 5: Synthesis of Intermediate 1-7

To a solution of Intermediate 1-6 (2.2 g, 3.35 mmol, 1 eq) in THF (22 mL) was added TBAF (1 M, 5.02 mL, 1.5 eq). The mixture was stirred at 25° C. for 2 hr. The residue was concentrated in vacuum. The crude product was purified by reversed-phase HPLC (0.1% FA condition). Intermediate 1-7 (1 g, 2.39 mmol, 62.04% yield) was obtained as a yellow oil. LCMS (Method E): Rt=0.478 min, [M+H]⁺=419.2. SFC: Rt=1.603 min, ee %=82.164%. ¹H NMR (400 MHz, CHLOROFORM-d) δ=7.38-7.35 (m, 5H), 6.50 (br s, 1H), 5.36 (br d, J=8.5 Hz, 1H), 5.10 (s, 2H), 4.58-4.32 (m, 4H), 3.94-3.80 (m, 2H), 3.78-3.70 (m, 2H), 3.35-3.21 (m, 3H), 2.02-1.97 (m, 2H), 1.76-1.66 (m, 7H), 1.09-0.84 (m, 4H).

### Step 6: Synthesis of Intermediate 1-8

To a solution of Intermediate 1-7 (500 mg, 1.19 mmol, 1 eq) in toluene (15 mL) was added DBU (218.25 mg, 1.43 mmol, 216.09 μL, 1.2 eq) and DPPA (394.53 mg, 1.43 mmol, 309.43 μL, 1.2 eq) at 0° C. The mixture was stirred at 25° C. for 1 hr, then the mixture was warmed to 60° C. for 4 hr. The mixture was poured into water (50 mL) and extracted with EA (20 mL*2). The combined organic phase was dried over anhydrous Na₂SO₄, filtered and concentrated under vacuum. The residue was purified by flash silica gel chromatography (ISCO; 40 g SepaFlash Silica Flash Column, Eluent of 0˜100% Ethyl acetate/Petroleum ether gradient @20 mL/min). Intermediate 1-8 (400 mg, 901.84 μmol, 75.49% yield) was obtained as a white solid. LCMS (Method E): Rt=0.541 min, [M+Na]⁺=466.3. ¹H NMR (400 MHz, CHLOROFORM-d) δ=7.41-7.32 (m, 6H), 6.03 (br s, 1H), 5.25 (br d, J=7.8 Hz, 1H), 5.11 (s, 2H), 4.44 (q, J=6.1 Hz, 4H), 3.90 (dd, J=6.8, 8.6 Hz, 1H), 3.41 (br t, J=7.0 Hz, 2H), 3.33-3.17 (m, 2H), 2.10-1.89 (m, 4H), 1.85-1.64 (m, 7H), 1.23-0.92 (m, 4H).

### Step 7: Synthesis of Intermediate 1-9

To a solution of PPh₃ (133.05 mg, 507.28 μmol, 1.5 eq) and H₂O (18.28 mg, 1.01 mmol, 18.28 μL, 3 eq) in THF (2 mL) was added Intermediate 1-8 (150 mg, 338.19 μmol, 1 eq) in THF (2 mL). The mixture was stirred at 25° C. for 16 hr. The mixture was combined with another lot of material (250 mg of Intermediate 1-8 starting material) and concentrated under vacuum. The crude product was purified by reversed-phase HPLC (0.1% FA condition). Intermediate 1-9 (200 mg, 479.00 μmol, 53.05% yield) was obtained as a white solid. LCMS (Method E): Rt=0.435 min, [M+H]⁺=418.3. ¹H NMR (400 MHz, CHLOROFORM-d) δ=7.41-7.28 (m, 5H), 6.67 (br t, J=5.4 Hz, 1H), 5.39 (br d, J=7.8 Hz, 1H), 5.10 (s, 2H), 4.41 (s, 3H), 3.97-3.87 (m, 1H), 3.43-3.13 (m, 2H), 2.87-2.70 (m, 2H), 1.86-1.57 (m, 7H), 1.27-0.94 (m, 5H).

### Step 8: Synthesis of Intermediate 1-11

To a solution of Intermediate 1-9 (200 mg, 479.00 μmol, 1 eq), Intermediate 1-10 (190.92 mg, 431.10 μmol, 0.9 eq) and KI (159.03 mg, 957.99 μmol, 2 eq) in DMF (4 mL) was added DIEA (123.81 mg, 957.99 μmol, 166.86 μL, 2 eq). The mixture was stirred at 40° C. for 2 hr. The mixture was poured into water (20 mL) and extracted with EA (20 mL*2). The combined organic phase was dried over anhydrous Na₂SO₄, filtered and concentrated under vacuum. The crude product was purified by reversed-phase HPLC (0.1% FA condition). Intermediate 1-11 (80 mg, 97.09 μmol, 20.27% yield) was obtained as a yellow solid. LCMS (Method E): Rt=0.469 min, [M+H]⁺=824.4. ¹H NMR (400 MHz, METHANOL-d₄) δ=8.41-8.33 (m, 1H), 7.97-7.91 (m, 1H), 7.85 (dtd, J=1.3, 7.4, 18.3 Hz, 2H), 7.53-7.44 (m, 1H), 7.41-7.23 (m, 6H), 7.16 (t, J=9.0 Hz, 1H), 5.07 (s, 2H), 4.40 (br d, J=17.6 Hz, 5H), 3.94-3.83 (m, 1H), 3.81-3.71 (m, 2H), 3.70-3.63 (m, 1H), 3.59-3.44 (m, 4H), 3.41 (br s, 1H), 2.74-2.51 (m, 2H), 2.01-1.80 (m, 4H), 1.80-1.55 (m, 7H), 1.22-1.00 (m, 4H).

### Step 9: Synthesis of Intermediate 1-12

To a solution of Intermediate 1-11 (70 mg, 84.96 μmol, 1 eq) in EtOH (3 mL) was added Boc₂O (22.25 mg, 101.95 μmol, 23.42 μL, 1.2 eq). The mixture was stirred at 25° C. for 2 hr. The mixture was concentrated in vacuum. The crude product was purified by reversed-phase HPLC (0.1% NH₃·H₂O). Intermediate 1-12 (50 mg, 54.11 μmol, 63.69% yield) was obtained as a white solid. LCMS (Method G): Rt=0.648 min, [M+H]⁺=924.6. SFC: Rt=1.224 min. ¹H NMR (400 MHz, METHANOL-d₄) δ=8.37 (d, J=7.5 Hz, 1H), 7.95 (br d, J=8.0 Hz, 1H), 7.91-7.79 (m, 2H), 7.54-7.45 (m, 1H), 7.44-7.24 (m, 6H), 7.16 (t, J=8.9 Hz, 1H), 5.07 (br s, 2H), 4.49-4.36 (m, 6H), 4.23-4.06 (m, 2H), 3.87-3.72 (m, 3H), 3.71-3.58 (m, 2H), 3.54-3.42 (m, 3H), 3.23 (br dd, J=1.8, 6.6 Hz, 2H), 2.03-1.86 (m, 4H), 1.82-1.56 (m, 7H), 1.52-1.38 (m, 9H), 1.31-0.94 (m, 5H).

### Step 10: Synthesis of Intermediate 1-13

To a solution of Intermediate 1-12 (40 mg, 43.29 μmol, 1 eq), TEA (6.57 mg, 64.93 μmol, 9.04 L, 1.5 eq) and PdCl₂ (2.15 mg, 12.12 μmol, 0.28 eq) in DCM (2 mL) was added Et₃SiH (50.33 mg, 432.87 μmol, 69.14 μL, 10 eq) dropwise at 0° C. The mixture was stirred at 25° C. for 2 hr. The mixture was filtered and the filtrate was concentrated under vacuum. The crude product was purified by reversed-phase HPLC (0.1% NH₃·H₂O). Intermediate 1-13 (20 mg, 25.32 μmol, 58.49% yield) was obtained as a yellow solid. LCMS (Method G): Rt=0.566 min, [M+H]⁺=790.5. SFC: Rt=2.278 min, ee %=84.335%. ¹H NMR (400 MHz, METHANOL-d₄) δ=8.37 (d, J=6.4 Hz, 1H), 7.95 (br d, J=4.5 Hz, 1H), 7.91-7.80 (m, 2H), 7.54-7.45 (m, 1H), 7.42-7.33 (m, 1H), 7.17 (t, J=9.1 Hz, 1H), 4.52-4.42 (m, 4H), 4.39 (s, 2H), 4.20 (br s, 1H), 4.16-4.10 (m, 1H), 3.87-3.75 (m, 2H), 3.72-3.60 (m, 2H), 3.56-3.45 (m, 3H), 3.29-3.19 (m, 5H), 3.10-3.00 (m, 1H), 2.05-1.87 (m, 4H), 1.80-1.58 (m, 6H), 1.52-1.40 (m, 8H), 1.29-1.00 (m, 5H).

### Step 11: Synthesis of Intermediate 1-15

To a solution of Intermediate 1-14 (8.41 mg, 20.89 μmol, 1.1 eq) in DMF (0.5 mL) was added HOAt (5.17 mg, 37.98 μmol, 5.31 μL, 2 eq) and EDCI (7.28 mg, 37.98 μmol, 2 eq). The mixture was stirred at 25° C. for 30 min, then Intermediate 1-13 (15 mg, 18.99 μmol, 1 eq) and NMM (5.76 mg, 56.97 mol, 6.26 μL, 3 eq) were added. The mixture was stirred at 25° C. for 2 hr. The mixture was purified directly without further work-up. The crude product was purified by reversed-phase HPLC (0.1% NH₃·H₂O). Intermediate 1-15 (16 mg, 13.62 μmol, 71.75% yield) was obtained as a white solid. LCMS (Method G): Rt=0.719 min, [M+H]⁺=1174.8. ¹H NMR (400 MHz, METHANOL-d₄) δ=8.41-8.32 (m, 1H), 8.01-7.91 (m, 1H), 7.90-7.81 (m, 2H), 7.80-7.75 (m, 1H), 7.74-7.67 (m, 1H), 7.54-7.32 (m, 4H), 7.16 (t, J=8.6 Hz, 1H), 4.60-4.50 (m, 1H), 4.49-4.41 (m, 4H), 4.40-4.36 (m, 2H), 4.35-4.27 (m, 1H), 4.25-4.15 (m, 2H), 4.15-4.07 (m, 1H), 4.06-3.97 (m, 1H), 3.95-3.85 (m, 1H), 3.84-3.71 (m, 2H), 3.70-3.54 (m, 3H), 3.52-3.40 (m, 5H), 2.82-2.65 (m, 3H), 2.12-1.64 (m, 14H), 1.53-1.36 (m, 18H), 1.33-1.21 (m, 3H), 1.16-1.02 (m, 2H), 0.76-0.52 (m, 4H).

### Step 12: Synthesis of I-458

To a solution of Intermediate 1-15 (15 mg, 12.77 μmol, 1 eq) in DCM (1 mL) was added TFA (230.25 mg, 2.02 mmol, 150.00 μL, 158.10 eq). The mixture was stirred at 25° C. for 2 hr. The mixture was basified to pH=8 with NH₃·H₂O, and concentrated under vacuum. The crude product was purified by reversed-phase HPLC (0.1% NH₃·H₂O). I-458 (11.3 mg, 10.34 μmol, 80.98% yield, 99.6% purity, TFA salt) was obtained as a white solid. LCMS (Method G): Rt=0.575 min, [M+H]⁺=974.7. SFC: Rt=4.847 min, 5.121 min, 7.513 min. ¹H NMR (400 MHz, METHANOL-d₄) δ=8.41-8.33 (m, 1H), 7.98-7.92 (m, 1H), 7.90-7.81 (m, 2H), 7.80-7.74 (m, 1H), 7.71 (br t, J=5.0 Hz, 1H), 7.52-7.40 (m, 3H), 7.39-7.33 (m, 1H), 7.21-7.11 (m, 1H), 4.60-4.52 (m, 1H), 4.52-4.39 (m, 4H), 4.38 (s, 2H), 4.30 (br dd, J=4.2, 8.3 Hz, 1H), 3.93-3.85 (m, 1H), 3.79-3.66 (m, 4H), 3.61 (br d, J=2.8 Hz, 2H), 3.58-3.47 (m, 3H), 3.45-3.33 (m, 4H), 3.23-3.09 (m, 2H), 2.86-2.67 (m, 4H), 2.32-2.23 (m, 1H), 2.06-1.97 (m, 3H), 1.93-1.75 (m, 8H), 1.73-1.63 (m, 3H), 1.32-1.07 (m, 5H), 0.52-0.39 (m, 4H). ¹⁹F NMR (377 MHz, METHANOL-d₄) δ=−76.93 (s, 1F), −120.72 (br d, J=13.5 Hz, 1F).

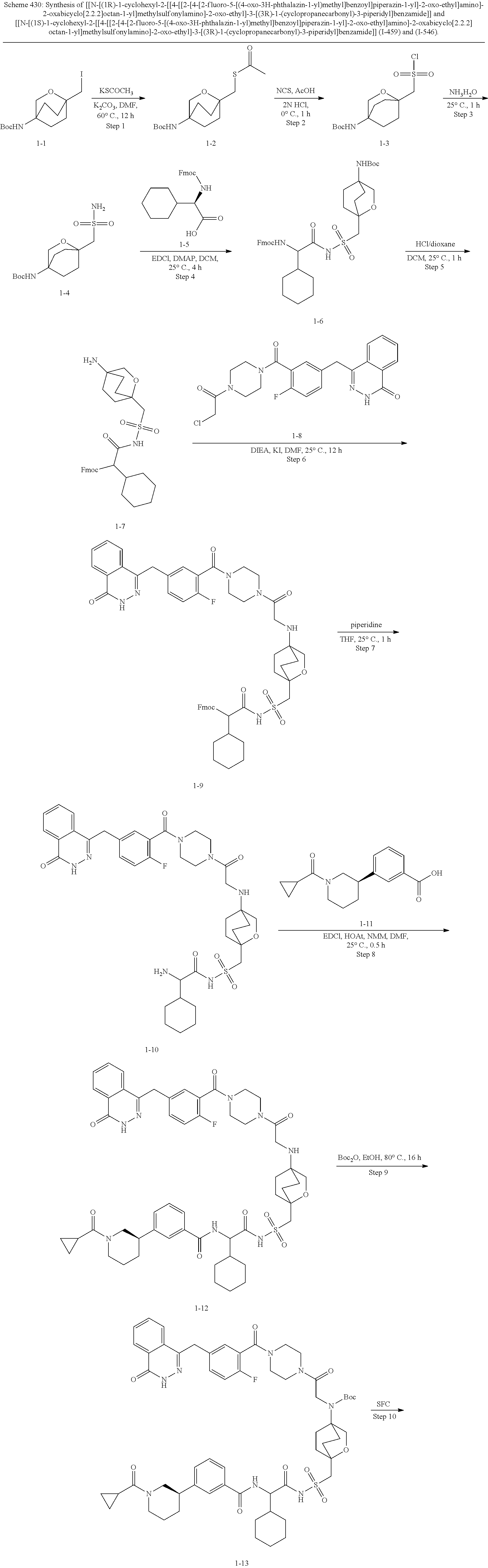

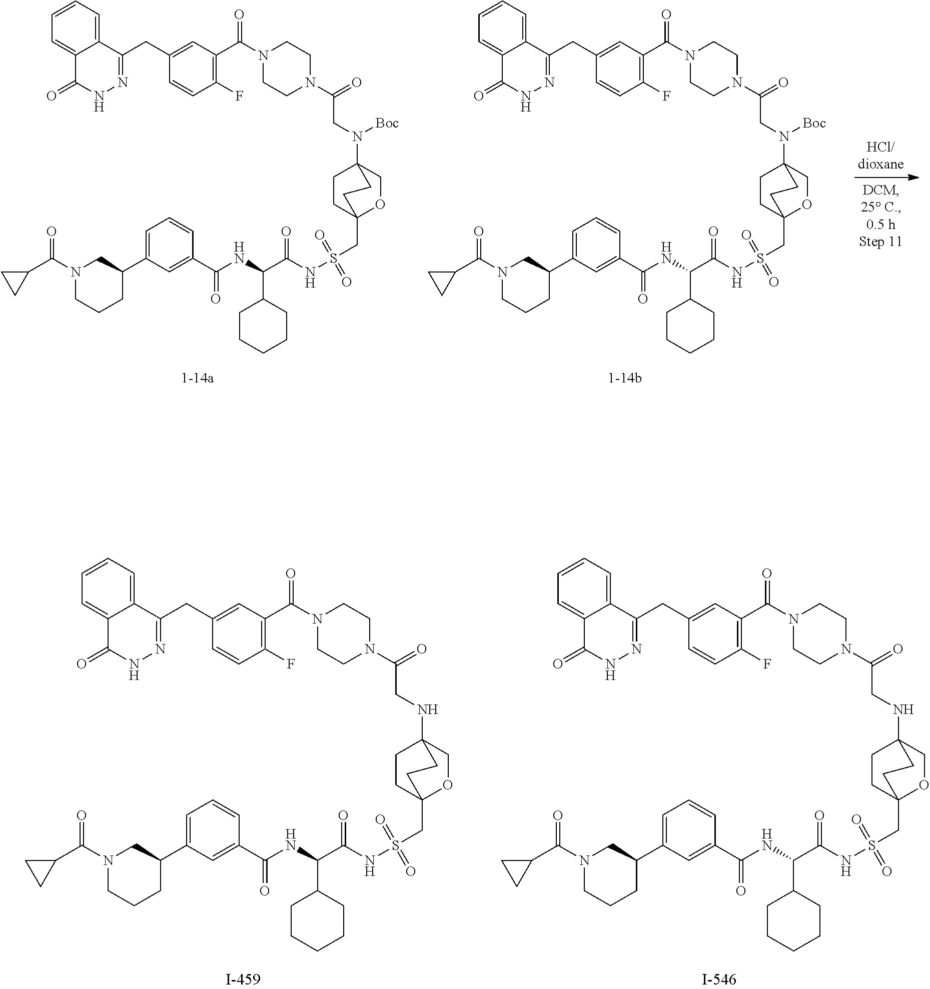

### Step 1: Synthesis of Intermediate 1-2

To a solution of intermediate 1-1 (1 g, 2.72 mmol, 1 eq) in DMF (10 mL) was added K₂CO₃ (376.37 mg, 2.72 mmol, 1 eq) and potassium;ethanethioate (373.21 mg, 3.27 mmol, 1.2 eq). The mixture was stirred at 60° C. for 4 hr. The reaction mixture was poured into H₂O (5 mL), and extracted with EA (5 mL*3). The combined organic layers were dried over Na₂SO₄, filtered and concentrated under reduced pressure to give a residue. The residue was purified by flash silica gel chromatography (ISCO; 20 g SepaFlash Silica Flash Column, Eluent of 0˜100% PE/EA@36 mL/min, PE/EA=10:1, Rf=0.2) and the eluent was concentrated under reduced pressure to give Intermediate 1-2 (822 mg, crude) as a white solid. LCMS (Method D): Rt=0.364 min, (M+H)=260.0.

### Step 2: Synthesis of Intermediate 1-3

To a solution of intermediate 1-2 (822 mg, 2.61 mmol, 1 eq) in CH₃CN (10 mL) and HCl (2 M, 4.85 mL, 3.72 eq) was added NCS (1.74 g, 13.03 mmol, 5 eq). The mixture was stirred at 0° C. for 2 hr. The reaction liquid is used directly for the next step. Intermediate 1-3 (800 mg, crude) was obtained as a yellow gum. LCMS (Method D): Rt=0.353 min, (M+H)=284.0.

### Step 3: Synthesis of Intermediate 1-4

To a solution of intermediate 1-3 (800 mg, 2.35 mmol, 1 eq) in CH₃CN (10 mL) was added NH₃—H₂O (4.55 g, 32.46 mmol, 5 mL, 25% purity, 13.79 eq). The mixture was stirred at 25° C. for 2 hr. The reaction mixture was poured into H₂O (5 mL), and extracted with EA (5 mL*3). The combined organic layers were dried over Na₂SO₄, filtered and concentrated under reduced pressure to give Intermediate 1-4 (1.3 g, crude) as a yellow solid. LCMS (Method C): Rt=0.631 min, (M+H)=265.0. ¹H NMR (400 MHz, CHLOROFORM-d) δ=5.02 (s, 2H), 4.53-4.26 (m, 1H), 4.07-3.96 (m, 2H), 3.20 (s, 2H), 2.44-2.29 (m, 2H), 2.21-2.07 (m, 2H), 1.86-1.74 (m, 4H), 1.48-1.37 (m, 9H).

### Step 4: Synthesis of Intermediate 1-6

To a solution of intermediate 1-4 (1.01 g, 3.15 mmol, 1 eq) and intermediate 1-5 (1.32 g, 3.47 mmol, 1.32 mL, 1.1 eq) in DCM (10 mL) was added EDCI (604.29 mg, 3.15 mmol, 1 eq) and DMAP (385.10 mg, 3.15 mmol, 1 eq). The mixture was stirred at 25° C. for 4 hr. The reaction mixture was poured into H₂O (5 mL), and extracted with DCM (5 mL*3). The combined organic layers were dried over Na₂SO₄, filtered and concentrated under reduced pressure to give a residue. The residue was purified by prep-HPLC (column: Phenomenex luna C18 150*25 mm*10 um; mobile phase: [water (FA)-ACN]; gradient: 60%-90% B over 10 min) and the eluent was concentrated to remove MeCN and then lyophilized. Intermediate 1-6 (700 mg, crude) was obtained as a white solid LCMS (Method D): Rt=0.533 min, (M+H)=626.3.

### Step 5: Synthesis of Intermediate 1-7

To a solution of intermediate 1-6 (450 mg, 659.98 μmol, 1 eq) in DCM (4.5 mL) was added HCl/dioxane (2 M, 419.21 μL, 1.79 eq). The mixture was stirred at 25° C. for 1 hr. The reaction mixture was concentrated under reduced pressure to give Intermediate 1-7 (480 mg, crude, HCl salt) as a yellow solid. LCMS (Method D): Rt=0.399 min, (M+H)=582.3.

### Step 6: Synthesis of Intermediate 1-9

To a solution of intermediate 1-7 (380 mg, 614.47 μmol, 1 eq, HCl salt), and intermediate 1-8 (200.59 mg, 452.94 μmol, 1 eq) in DMF (3.8 mL) was added DIEA (117.08 mg, 905.88 μmol, 157.78 μL, 2 eq) and KI (75.19 mg, 452.94 μmol, 1 eq). The mixture was stirred at 25° C. for 3 hr. The reaction mixture was concentrated under reduced pressure to give a residue. The residue was purified by reversed-phase chromatography (FA condition) and the eluent was concentrated to remove MeCN and then lyophilized. Intermediate 1-9 (350 mg, crude) was obtained as a brown solid. LCMS (Method D): Rt=0.406 min, (M+H)=988.3.

### Step 7: Synthesis of Intermediate 1-10

To a solution of intermediate 1-9 (350 mg, 354.20 μmol, 1 eq) in THF (3.6 mL) was added piperidine (21.54 mg, 253.00 μmol, 24.99 μL, 1 eq). The mixture was stirred at 25° C. for 0.5 hr. The reaction mixture was concentrated under reduced pressure to give a residue. The residue was purified by reversed-phase chromatography (basic condition) and the eluent was concentrated to remove MeCN and then lyophilized. Intermediate 1-10 (180 mg, 235.02 μmol, 92.89% yield) was obtained as a brown gum. LCMS (Method D): Rt=0.259 min, (M+H)=766.3.

### Step 8: Synthesis of Intermediate 1-12

To a solution of intermediate 1-10 (95 mg, 124.04 μmol, 1 eq), intermediate 1-11 (42.82 mg, 156.68 μmol, 1.5 eq) in DMF (1 mL) was added EDCI (60.07 mg, 313.36 μmol, 3 eq), HOAt (14.22 mg, 104.45 μmol, 14.61 μL, 1 eq) and NMM (52.83 mg, 522.27 μmol, 57.42 μL, 5 eq). The mixture was stirred at 25° C. for 0.5 hr. The reaction mixture was poured into H₂O (5 mL), and extracted with EA (5 mL*3). The combined organic layers were dried over Na₂SO₄, filtered and concentrated under reduced pressure to give a residue. The residue was purified by reversed-phase chromatography (FA condition) and the eluent was concentrated to remove MeCN and then lyophilized. Intermediate 1-12 (150 mg, crude) was obtained as a yellow solid. LCMS (Method D): Rt=0.375 min, (M+H)=1021.3.

### Step 9: Synthesis of Intermediate 1-13

To a solution of intermediate 1-12 (100 mg, 97.92 μmol, 1 eq) in EtOH (0.1 mL) was added Boc₂O (28.21 mg, 129.26 μmol, 29.70 μL, 2 eq). The mixture was stirred at 80° C. for 5 hr. The reaction mixture was concentrated under reduced pressure to give a residue. The residue was purified by reversed-phase chromatography (FA condition) and the eluent was concentrated to remove MeCN and then lyophilized. Intermediate 1-13 (80 mg, 71.34 μmol, 68.99% yield) was obtained as a white solid. LCMS (Method D): Rt=0.462 min, (M+H)=1121.4.

Step 10: Synthesis of Intermediate 1-14a & intermediate 1-14b

Intermediate 1-13 was purified by prep-HPLC (column: DAICEL CHIRALPAK IK (250 mm*30 mm, 10 um); mobile phase: [CO₂-ACN/EtOH (0.1% NH₃H₂O)]; gradient: 7%-60% B over 5.3 min) and the eluent was concentrated to remove MeCN and then lyophilized. Intermediate 1-14a (40 mg, 35.67 μmol, 80.00% yield) was obtained as a yellow solid. Intermediate 1-14b (15 mg, 13.38 μmol, 30.00% yield) was obtained as a yellow solid. LCMS (Method D): Rt=0.460 min, (M+H)=1121.4.

### Step 11: Synthesis of I-459 and I-546

To a solution of intermediate 1-14a (40 mg, 35.67 μmol, 1 eq) in DCM (0.4 mL) was added HCl/dioxane (2 M, 0.2 mL, 11.21 eq). The mixture was stirred at 25° C. for 0.5 hr. The reaction mixture was concentrated under reduced pressure to give a residue. The residue was purified by reversed-phase chromatography (FA condition) and the eluent was concentrated to remove MeCN and then lyophilized. I-459 (9.61 mg, 9.41 μmol, 26.38% yield, 100% purity) was obtained as a white solid.

LCMS: Rt=0.386 min, (M+H)=1021.4.

LCMS: Rt=0.381 min, (M+H)=1021.4.

¹H NMR (400 MHz, METHANOL-d4) δ=8.37 (br d, J=8.0 Hz, 1H), 7.99-7.91 (m, 1H), 7.91-7.82 (m, 2H), 7.82-7.77 (m, 1H), 7.73 (d, J=2.8 Hz, 1H), 7.56-7.41 (m, 3H), 7.40-7.34 (m, 1H), 7.24-7.11 (m, 1H), 4.61-4.53 (m, 1H), 4.39 (s, 4H), 3.83-3.71 (m, 5H), 3.66 (s, 2H), 3.57-3.50 (m, 2H), 3.43 (s, 2H), 3.38 (s, 4H), 2.89-2.70 (m, 2H), 2.17-2.04 (m, 5H), 2.01 (d, J=2.4 Hz, 1H), 1.91 (d, J=10.0 Hz, 2H), 1.88-1.75 (m, 9H), 1.74-1.62 (m, 2H), 1.33-1.11 (m, 5H), 0.91-0.74 (m, 4H).

F NMR (400 MHz, METHANOL-d4) δ=−120.755.

LCMS: Rt=0.375 min, (M+H)=1021.4.

¹H NMR (400 MHz, METHANOL-d4) δ=8.39-8.35 (m, 1H), 7.98-7.92 (m, 1H), 7.86 (d, J=9.6 Hz, 1H), 7.82 (d, J=6.8 Hz, 1H), 7.80-7.67 (m, 2H), 7.56-7.41 (m, 3H), 7.41-7.33 (m, 1H), 7.17 (t, J=8.8 Hz, 1H), 4.60-4.53 (m, 2H), 4.39 (s, 4H), 3.83-3.68 (m, 6H), 3.65 (d, J=17.2 Hz, 2H), 3.57-3.46 (m, 3H), 3.44-3.38 (m, 3H), 2.90-2.69 (m, 2H), 2.15-2.05 (m, 5H), 2.03-1.97 (m, 1H), 1.93-1.88 (m, 2H), 1.84 (s, 3H), 1.81 (d, J=7.6 Hz, 4H), 1.76 (s, 2H), 1.73-1.63 (m, 2H), 1.31-1.13 (m, 5H), 0.91-0.78 (m, 4H)

F NMR (400 MHz, METHANOL-d4) δ=−120.757.

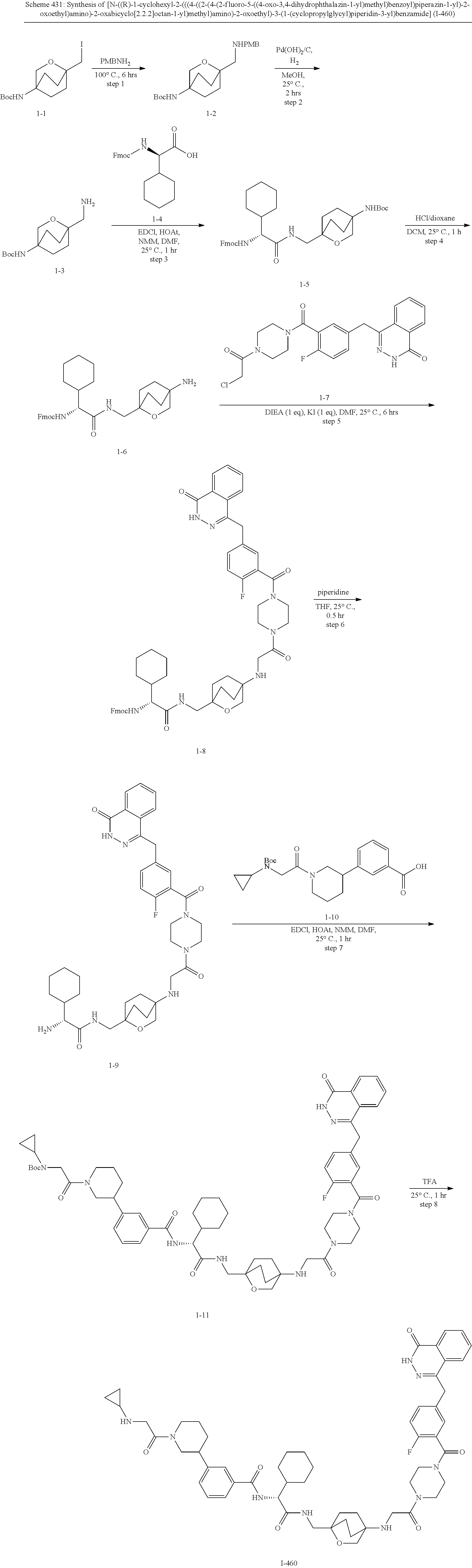

### Step 1: Synthesis of Intermediate 1-2.

To a solution of Intermediate 1-1 (1.5 g, 4.08 mmol, 1 eq) was added (4-methoxyphenyl)methanamine (1.12 g, 8.17 mmol, 1.06 mL, 2 eq). The mixture was stirred at 100° C. for 6 hr. The residue was purified by prep-HPLC (FA condition), and the eluent was concentrated and lyophilized to give the desired product. Intermediate 1-2 (1.1 g, 2.02 mmol, 49.36% yield, 77.436% purity, FA salt) was obtained as a white solid. LCMS (Method D): Retention time: 0.348 min, (M+H)=337.0. ¹H NMR (400 MHz, METHANOL-d4) δ=7.39 (d, J=8.8 Hz, 2H), 7.03-6.96 (m, 2H), 4.11 (s, 2H), 3.95 (s, 2H), 3.82 (s, 3H), 2.84 (s, 2H), 2.13-2.04 (m, 2H), 1.99-1.83 (m, 4H), 1.75-1.64 (m, 2H), 1.40 (s, 9H).

### Step 2: Synthesis of Intermediate 1-3.

To a solution of Pd(OH)₂/C (600 mg, 20% purity) in MeOH (10 mL) was added Intermediate 1-2 (1 g, 2.37 mmol, 1 eq, FA salt), and the mixture was degassed and purged with H₂ 3 times, and then the mixture was stirred at 25° C. for 2 hr under H₂ (15 psi) atmosphere. The reaction mixture was filtered and washed with MeOH (15 mL*2), the filtrate was concentrated under reduced pressure to give Intermediate 1-3 (360 mg, 1.09 mmol, 45.93% yield, 77.402% purity) as a colorless oil. LCMS (Method D): Retention time: 0.178 min, (M+H)=257.2. ¹H NMR (400 MHz, METHANOL-d4) δ=3.91 (s, 2H), 2.48 (s, 2H), 2.09-2.01 (m, 2H), 1.93-1.83 (m, 4H), 1.70-1.60 (m, 2H), 1.41 (s, 9H).

### Step 3: Synthesis of Intermediate 1-5.

To a solution of Intermediate 1-3 (320 mg, 1.25 mmol, 1 eq) and Intermediate 1-4 (473.68 mg, 1.25 mmol, 473.68 μL, 1 eq) in DMF (3 mL) was added HOAt (169.91 mg, 1.25 mmol, 174.63 μL, 1 eq), EDCI (717.92 mg, 3.75 mmol, 3 eq) and NMM (631.33 mg, 6.24 mmol, 686.23 μL, 5 eq). The mixture was stirred at 25° C. for 1 hr. The reaction mixture was diluted with H₂O (5 mL) and extracted with EA (5 mL*3). The combined organic layers were washed with brine (5 mL*3), dried over Na₂SO₄, filtered and concentrated under reduced pressure to give a residue. The residue was purified by flash silica gel chromatography (ISCO; 20 g SepaFlash Silica Flash Column, Eluent of 0˜10% Methanol/Ethyl acetate gradient @60 mL/min), the eluent was concentrated under reduced pressure to give Intermediate 1-5 (570 mg, 881.59 μmol, 70.62% yield, 95.548% purity) as a pale yellow solid. LCMS (Method D): Retention time: 0.531 min, (M+H)=618.3. SFC: Retention time: 0.949 min. ¹H NMR (400 MHz, METHANOL-d4) δ=7.80 (d, J=7.6 Hz, 2H), 7.71-7.62 (m, 2H), 7.43-7.36 (m, 2H), 7.34-7.28 (m, 2H), 4.44-4.35 (m, 2H), 4.27-4.17 (m, 1H), 3.95-3.83 (m, 3H), 3.16-3.12 (m, 1H), 2.05-1.93 (m, 2H), 1.84-1.73 (m, 6H), 1.70-1.51 (m, 6H), 1.40 (s, 9H), 1.31-1.18 (m, 4H), 1.10-0.97 (m, 2H).

### Step 4: Synthesis of Intermediate 1-6.

To a solution of Intermediate 1-5 (550 mg, 890.29 μmol, 1 eq) in DCM (3 mL) was added HCl/dioxane (2 M, 3 mL). The mixture was stirred at 25° C. for 1 hr. The reaction mixture was concentrated under reduced pressure to give Intermediate 1-6 (480 mg, 661.20 μmol, 74.27% yield, 76.330% purity, HCl salt) as a white solid. LCMS (Method D): Retention time: 0.383 min, (M+H)=518.3. ¹HNMR (400 MHz, METHANOL-d4) δ=7.81 (d, J=7.6 Hz, 2H), 7.67 (d, J=7.2 Hz, 2H), 7.44-7.36 (m, 2H), 7.34-7.28 (m, 2H), 4.40-4.17 (m, 3H), 3.89 (d, J=7.6 Hz, 1H), 3.79 (s, 2H), 3.25-3.11 (m, 2H), 1.94-1.62 (m, 14H), 1.34-1.17 (m, 5H).

### Step 5: Synthesis of Intermediate 1-8.

To a solution of Intermediate 1-6 (400 mg, 721.87 μmol, 1 eq, HCl salt) and Intermediate 1-7 (319.69 mg, 721.87 μmol, 1 eq) in DMF (4 mL) was added DIEA (93.30 mg, 721.87 μmol, 125.74 μL, 1 eq) and KI (119.83 mg, 721.87 μmol, 1 eq). The mixture was stirred at 25° C. for 6 hr. The reaction mixture was diluted with H₂O (5 mL) and extracted with EA (10 mL*3). The combined organic layers were washed with brine (10 mL*3), and concentrated under reduced pressure to give a residue. The residue was purified by prep-HPLC (FA condition), the eluent was concentrated and lyophilized to give Intermediate 1-8 (110 mg, 109.51 μmol, 15.17% yield, 96.579% purity, FA salt) as a white solid. LCMS (Method D): Retention time: 0.403 min, (M+H)=924.4. SFC: Retention time: 2.131 min. ¹H NMR (400 MHz, METHANOL-d4) δ=8.45 (s, 1H), 8.37 (d, J=7.4 Hz, 1H), 7.97-7.72 (m, 5H), 7.70-7.59 (m, 2H), 7.54-7.44 (m, 1H), 7.44-7.22 (m, 5H), 7.22-7.11 (m, 1H), 4.83 (s, 1H), 4.44-4.31 (m, 4H), 4.26-4.19 (m, 1H), 3.95-3.84 (m, 1H), 3.80-3.57 (m, 5H), 3.50 (s, 3H), 3.40 (s, 2H), 3.29-3.23 (m, 1H), 3.21-3.10 (m, 2H), 1.95-1.52 (m, 14H), 1.32-1.15 (m, 3H), 1.13-0.96 (m, 2H).

### Step 6: Synthesis of Intermediate 1-9.

To a solution of Intermediate 1-8 (100 mg, 103.08 μmol, 1 eq, FA salt) in THF (0.8 mL) was added piperidine (0.1 mL). The mixture was stirred at 25° C. for 0.5 hr. The reaction mixture was concentrated under reduced pressure to give a residue. The residue was purified by prep-HPLC (neutral condition), and the eluent was concentrated and lyophilized to give Intermediate 1-9 (160 mg, crude) as a yellow solid. LCMS (Method D): Retention time: 0.299 min, (M+H)=702.3. SFC: Retention time: 0.951 min.

### Step 7: Synthesis of Intermediate 1-11.

To a solution of Intermediate 1-9 (20 mg, 28.50 μmol, 1 eq) and Intermediate 1-10 (11.47 mg, 28.50 μmol, 1 eq) in DMF (0.2 mL) was added HOAt (3.88 mg, 28.50 μmol, 3.99 μL, 1 eq), EDCI (16.39 mg, 85.49 μmol, 3 eq), and NMM (14.41 mg, 142.49 μmol, 15.67 μL, 5 eq). The mixture was stirred at 25° C. for 1 hr. The reaction mixture was diluted with H₂O (5 mL) and extracted with EA (5 mL*3). The combined organic layers were washed with brine (5 mL*3), dried over Na₂SO₄, filtered and concentrated under reduced pressure to give a residue. The crude product was purified by reversed-phase HPLC (neutral condition), the eluent was concentrated and lyophilized to give Intermediate 1-11 (25 mg, 18.37 μmol, 64.48% yield, 79.841% purity) as a yellow solid. LCMS (Method D): Retention time: 0.428 min, (M+H)=1086.7.

### Step 8: Synthesis of I-460

To a solution of Intermediate 1-11 (20 mg, 18.41 μmol, 1 eq) in DCM (0.2 mL) was added TFA (0.04 mL). The mixture was stirred at 25° C. for 0.5 hr. The mixture was adjusted to pH=8 with NH₃H₂O and concentrated under reduced pressure to give a residue. The crude product was purified by reversed-phase HPLC (neutral condition), the eluent was concentrated and lyophilized to give I-460 (6.92 mg, 6.81 μmol, 36.97% yield, 97.003% purity) as a white solid. LCMS (Method H): Retention time: 0.592 min, (M+H)=986.7. SFC: Retention time: 3.768, 5.348 min. ¹H NMR (400 MHz, METHANOL-d4) δ=8.37 (d, J=7.2 Hz, 1H), 7.97-7.92 (m, 1H), 7.91-7.80 (m, 2H), 7.79-7.67 (m, 2H), 7.53-7.34 (m, 4H), 7.19-7.11 (m, 1H), 4.89 (s, 1H), 4.62-4.55 (m, 1H), 4.39 (s, 3H), 3.95-3.86 (m, 1H), 3.80-3.73 (m, 2H), 3.67 (s, 3H), 3.61-3.43 (m, 6H), 3.39 (s, 2H), 3.23-3.09 (m, 3H), 2.88-2.69 (m, 2H), 2.29-2.21 (m, 1H), 2.11-2.03 (m, 1H), 1.87 (d, J=5.2 Hz, 6H), 1.79 (d, J=15.2 Hz, 4H), 1.73-1.59 (m, 7H), 1.40-1.20 (m, 4H), 1.19-1.08 (m, 2H), 0.52-0.37 (m, 4H). ¹⁹F NMR (376 MHz, METHANOL-d4) δ=−120.71.

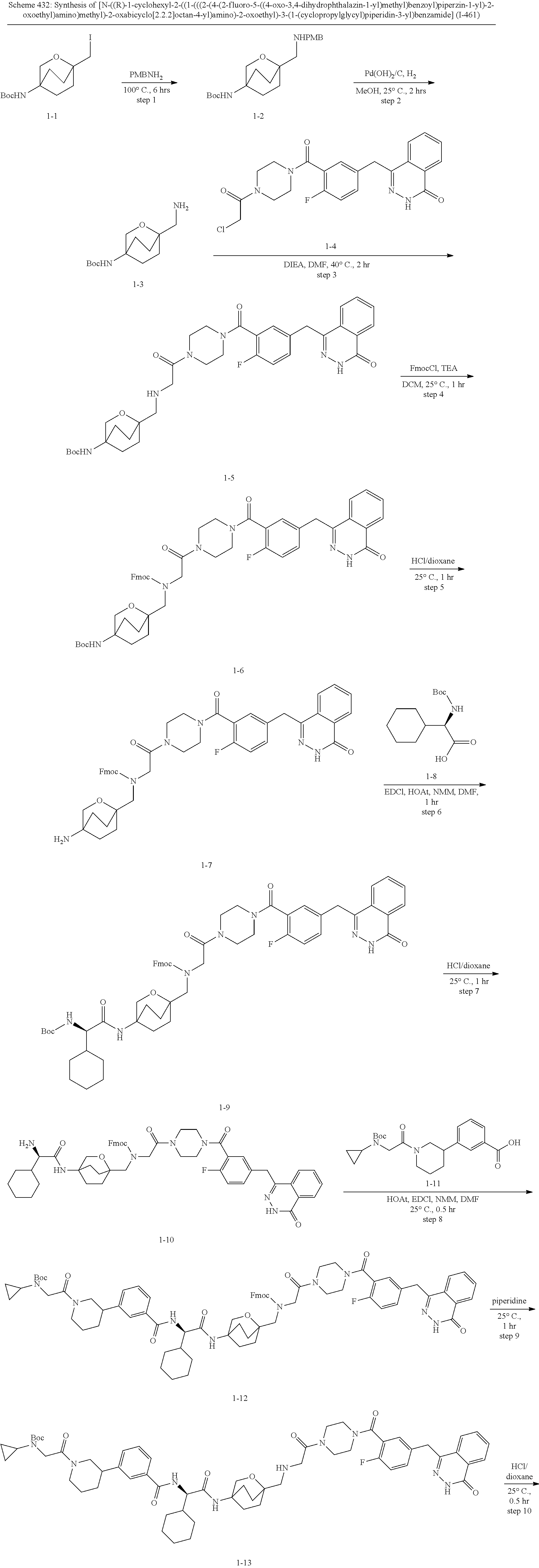

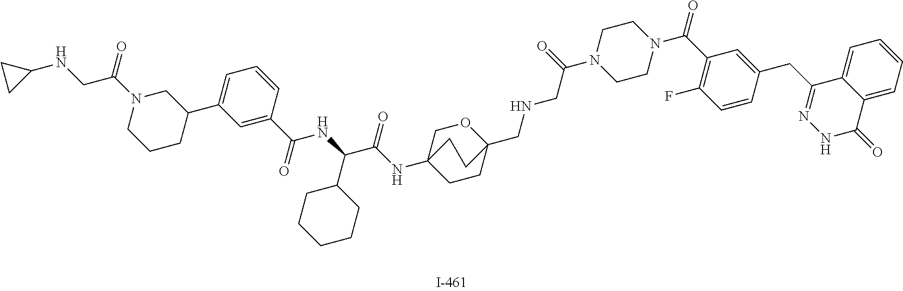 [SMILES: O=C(N[C@@H](C(=O)NC12CCC(CNCC(=O)N3CCN(C(=O)c4cc(Cc5n[nH]c(=O)c6ccccc56)ccc4F)CC3)(CC1)OC2)C1CCCCC1)c1cccc(C2CCCN(C(=O)CNC3CC3)C2)c1]

### Step 1: Synthesis of Intermediate 1-2.

To a solution of Intermediate 1-1 (1.2 g, 3.27 mmol, 1 eq) was added (4-methoxyphenyl)methanamine (896.54 mg, 6.54 mmol, 848.19 μL, 2 eq). The mixture was stirred at 100° C. for 6 hr. The reaction mixture was concentrated under reduced pressure to give a residue. The residue was purified by prep-HPLC (FA condition), the eluent was concentrated and lyophilized to give Intermediate 1-2 (900 mg, 2.08 mmol, 63.52% yield, 97.451% purity, FA salt) as a white solid. LCMS (Method D): Retention time: 0.316 min, (M+H)=337.2. ¹H NMR (400 MHz, METHANOL-d4) δ=8.53 (s, 1H), 7.42-7.34 (m, 2H), 7.03-6.93 (m, 2H), 4.10 (s, 2H), 3.95 (s, 2H), 3.84-3.80 (m, 3H), 2.84 (s, 2H), 2.14-2.05 (m, 2H), 1.96-1.81 (m, 4H), 1.76-1.66 (m, 2H), 1.41 (s, 9H).

### Step 2: Synthesis of Intermediate 1-3.

To a solution of Pd(OH)₂/C (900 mg, 424.98 μmol, 21.25 μL, 20% purity) in MeOH (9 mL) was added Intermediate 1-2 (900 mg, 2.13 mmol, 1 eq, FA salt), and the mixture was degassed and purged with H₂ 3 times, and then the mixture was stirred at 25° C. for 2 hr under H₂ (15 psi) atmosphere. The reaction mixture was filtered and washed with MeOH (15 mL*2). The filtrate was concentrated under reduced pressure to give Intermediate 1-3 (600 mg, crude) as a white solid. LCMS (Method D): Retention time: 0.196 min, (M+H)=257.2. ¹H NMR (400 MHz, METHANOL-d4) δ=3.91 (s, 2H), 3.78-3.70 (m, 1H), 2.48 (s, 2H), 2.10-1.99 (m, 2H), 1.93-1.80 (m, 4H), 1.71-1.56 (m, 2H), 1.41 (s, 9H).

### Step 3: Synthesis of Intermediate 1-5.

To a solution of Intermediate 1-3 (300 mg, 1.17 mmol, 1 eq) and Intermediate 1-4 (518.30 mg, 1.17 mmol, 1 eq) in DMF (3 mL) was added DIEA (453.76 mg, 3.51 mmol, 611.54 μL, 3 eq). The mixture was stirred at 40° C. for 2 hr. The reaction mixture was diluted with H₂O (5 mL) and extracted with EA (10 mL*3). The combined organic layers were washed with brine (15 mL*2), dried over Na₂SO₄, filtered and concentrated under reduced pressure to give a residue. The residue was purified by flash silica gel chromatography (ISCO; 20 g SepaFlash Silica Flash Column, Eluent of 0˜10% Methanol/Ethyl acetate gradient @60 mL/min), and the eluent was concentrated under reduced pressure to give Intermediate 1-5 (420 mg, 543.24 μmol, 46.42% yield, 85.722% purity) as a yellow oil. LCMS (Method D): Retention time: 0.311 min, (M+H)=663.3. ¹H NMR (400 MHz, METHANOL-d4) δ=8.42-8.32 (m, 1H), 8.00-7.93 (m, 1H), 7.90-7.80 (m, 2H), 7.53-7.35 (m, 2H), 7.21-7.11 (m, 1H), 4.88 (s, 1H), 4.39 (s, 2H), 3.92 (s, 2H), 3.83-3.74 (m, 2H), 3.69 (d, J=5.6 Hz, 1H), 3.61-3.47 (m, 4H), 3.43-3.36 (m, 2H), 2.56 (d, J=9.2 Hz, 2H), 2.11-1.93 (m, 4H), 1.89-1.81 (m, 2H), 1.74-1.64 (m, 2H), 1.41 (s, 9H).

### Step 4: Synthesis of Intermediate 1-6.

To a solution of Intermediate 1-5 (400 mg, 603.55 μmol, 1 eq) in DCM (4 mL) was added TEA (183.22 mg, 1.81 mmol, 252.02 μL, 3 eq) and Fmoc-Cl (171.75 mg, 663.90 μmol, 1.1 eq). The mixture was stirred at 25° C. for 1 hr. The reaction mixture was diluted with H₂O (5 mL) and extracted with EA (5 mL*3). The combined organic layers were dried over Na₂SO₄, filtered and concentrated under reduced pressure to give a residue. The residue was purified by flash silica gel chromatography (ISCO; 20 g SepaFlash Silica Flash Column, Eluent of 0˜15% Methanol/Ethyl acetate gradient @60 mL/min), the eluent was concentrated under reduced pressure to give Intermediate 1-6 (170 mg, 181.83 μmol, 30.13% yield, 94.658% purity) as a white solid. LCMS (Method D): Retention time: 0.502 min, (M+H)=885.6. ¹H NMR (400 MHz, METHANOL-d4) δ=8.38 (d, J=8.0 Hz, 1H), 7.96 (d, J=6.8 Hz, 1H), 7.89 (d, J=6.0 Hz, 1H), 7.87-7.80 (m, 3H), 7.63 (d, J=7.2 Hz, 1H), 7.58 (s, 1H), 7.45-7.26 (m, 6H), 7.24-7.14 (m, 1H), 4.72 (d, J=3.6 Hz, 2H), 4.67-4.59 (m, 2H), 4.44-4.38 (m, 2H), 4.26 (br s, 1H), 4.21-4.13 (m, 1H), 3.94-3.79 (m, 2H), 3.77-3.61 (m, 4H), 3.55-3.44 (m, 2H), 3.15 (s, 1H), 3.08 (s, 1H), 2.68 (s, 1H), 2.06-1.95 (m, 1H), 1.87-1.66 (m, 4H), 1.63-1.54 (m, 2H), 1.42 (s, 9H), 0.83-0.71 (m, 1H). ¹⁹F NMR (376 MHz, METHANOL-d4) δ=−120.79.

### Step 5: Synthesis of Intermediate 1-7.

To a solution of Intermediate 1-6 (170 mg, 192.09 μmol, 1 eq) in DCM (1 mL) was added HCl/dioxane (2 M, 1 mL). The mixture was stirred at 25° C. for 1 hr. The reaction mixture was concentrated under reduced pressure to give Intermediate 1-7 (180 mg, crude, HCl salt) as a white solid. LCMS (Method D): Retention time: 0.349 min, (M+H)=785.4.

### Step 6: Synthesis of Intermediate 1-9.

To a solution of Intermediate 1-8 (50 mg, 194.31 μmol, 1 eq) and Intermediate 1-7 (159.59 mg, 194.31 μmol, 1 eq, HCl salt) in DMF (1 mL) was added HOAt (26.45 mg, 194.31 μmol, 27.18 μL, 1 eq), EDCI (111.75 mg, 582.92 μmol, 3 eq) and NMM (98.27 mg, 971.53 μmol, 106.81 μL, 5 eq). The mixture was stirred at 25° C. for 1 hr. The reaction mixture was diluted with H₂O (5 mL) and extracted with EA (5 mL*3). The combined organic layers were washed with brine (5 mL*3), dried over Na₂SO₄, filtered and concentrated under reduced pressure to give a residue. The residue was purified by flash silica gel chromatography (ISCO; 20 g SepaFlash Silica Flash Column, Eluent of 0˜80% Ethyl acetate/Petroleum ether gradient @60 mL/min), the eluent was concentrated under reduced pressure to give Intermediate 1-9 (150 mg, 134.13 μmol, 69.03% yield, 91.580% purity) as a white solid. LCMS (Method D): Retention time: 0.589 min, (M+H)=1024.5. SFC: Retention time: 1.831 min. ¹H NMR (400 MHz, METHANOL-d4) δ=8.39 (d, J=7.6 Hz, 1H), 7.96 (d, J=7.2 Hz, 1H), 7.91-7.82 (m, 3H), 7.65-7.57 (m, 2H), 7.49 (s, 1H), 7.46-7.26 (m, 5H), 7.17 (d, J=9.2 Hz, 1H), 6.47-6.40 (m, 1H), 4.74 (d, J=3.2 Hz, 1H), 4.67-4.59 (m, 1H), 4.46-4.36 (m, 2H), 4.28-4.04 (m, 2H), 3.94-3.80 (m, 2H), 3.79-3.60 (m, 4H), 3.58-3.40 (m, 2H), 3.37 (s, 2H), 3.28-3.07 (m, 3H), 2.68 (s, 1H), 2.16-2.04 (m, 1H), 1.96-1.84 (m, 2H), 1.81-1.54 (m, 9H), 1.48-1.41 (m, 9H), 1.37-1.15 (m, 4H), 1.11-0.92 (m, 2H), 0.83-0.72 (m, 1H).

### Step 7: Synthesis of Intermediate 1-10.

To a solution of Intermediate 1-9 (140 mg, 136.69 μmol, 1 eq) in DCM (1 mL) was added HCl/dioxane (2 M, 1 mL). The mixture was stirred at 25° C. for 1 hr. The reaction mixture was concentrated under reduced pressure to give Intermediate 1-10 (180 mg, crude, HCl salt) as a white solid. LCMS (Method D): Retention time: 0.387 min, (M+H)=924.4.

### Step 8: Synthesis of Intermediate 1-12.

To a solution of Intermediate 1-11 (50 mg, 124.23 μmol, 1 eq) in DMF (1 mL) was added HOAt (16.91 mg, 124.23 μmol, 17.38 μL, 1 eq) and EDCI (71.44 mg, 372.69 μmol, 3 eq) and the mixture was stirred for 0.5 hr. Then Intermediate 1-10 (119.33 mg, 124.23 μmol, 1 eq, HCl salt) and NMM (62.83 mg, 621.14 μmol, 68.29 μL, 5 eq) was added into the system. The mixture was stirred at 25° C. for 1 hr. The reaction mixture was diluted with H₂O (5 mL) and extracted with EA (5 mL*3). The combined organic layers were washed with brine (5 mL*3), dried over Na₂SO₄, filtered and concentrated under reduced pressure to give a residue. The residue was purified by flash silica gel chromatography (ISCO; 20 g SepaFlash Silica Flash Column, Eluent of 0˜75% Ethyl acetate/Petroleum ether gradient @60 mL/min), and the eluent was concentrated under reduced pressure to give Intermediate 1-12 (70 mg, 49.21 μmol, 39.61% yield, 91.986% purity) as a pale yellow solid. LCMS (Method D): Retention time: 0.558 min, (M+H)=1208.7. SFC: Retention time: 3.476, 4.469 min. ¹H NMR (400 MHz, METHANOL-d4) δ=8.52 (d, J=5.8 Hz, 1H), 8.17-8.07 (m, 1H), 8.03 (d, J=7.6 Hz, 1H), 8.01-7.83 (m, 5H), 7.78 (d, J=7.6 Hz, 1H), 7.73 (s, 1H), 7.69-7.42 (m, 8H), 7.39-7.26 (m, 1H), 4.91-4.78 (m, 2H), 4.77-4.61 (m, 2H), 4.60-4.50 (m, 2H), 4.47-4.14 (m, 5H), 4.13-3.86 (m, 5H), 3.85-3.51 (m, 5H), 3.47-3.33 (m, 5H), 3.27-3.14 (m, 1H), 2.95-2.78 (m, 3H), 2.68-2.49 (m, 1H), 2.38-2.18 (m, 2H), 2.16-1.71 (m, 13H), 1.65-1.56 (m, 9H), 1.49-1.34 (m, 3H), 1.32-1.15 (m, 2H), 1.01-0.78 (m, 4H).

### Step 9: Synthesis of Intermediate 1-13.

To a solution of Intermediate 1-12 (60 mg, 45.85 μmol, 1 eq) in THF (0.8 mL) was added piperidine (0.1 mL). The mixture was stirred at 25° C. for 1 hr. The reaction mixture was diluted with H₂O (5 mL) and extracted with EA (5 mL*3). The combined organic layers were concentrated under reduced pressure to give Intermediate 1-12 (50 mg, crude) as a yellow oil. LCMS (Method D): Retention time: 0.436 min, (M+H)=1086.7.

### Step 10: Synthesis of I-461

To a solution of Intermediate 1-12 (50 mg, 46.03 μmol, 1 eq) in DCM (0.5 mL) was added HCl/dioxane (2 M, 0.5 mL). The mixture was stirred at 25° C. for 0.5 hr. The reaction mixture was concentrated under reduced pressure to give a residue. The residue was purified by prep-HPLC (HCl condition), the eluent was concentrated and lyophilized to give the desired product. The residue was purified by prep-HPLC (FA condition), and the eluent was concentrated and lyophilized to give I-461 (11.3 mg, 10.48 μmol, 22.76% yield, 95.701% purity, FA salt) as a white solid. LCMS (Method H): Retention time: 0.606 min, (M+H)=986.7. ¹H NMR (400 MHz, METHANOL-d4) δ=8.49 (s, 1H), 8.42-8.35 (m, 1H), 8.02-7.91 (m, 1H), 7.91-7.80 (m, 2H), 7.78-7.68 (m, 2H), 7.53-7.41 (m, 3H), 7.38-7.33 (m, 1H), 7.20-7.11 (m, 1H), 4.83 (s, 1H), 4.63-4.51 (m, 1H), 4.38 (s, 2H), 4.32-4.25 (m, 1H), 4.11-3.94 (m, 2H), 3.89-3.64 (m, 8H), 3.54 (s, 2H), 3.37 (d, J=8.0 Hz, 2H), 3.28-3.13 (m, 1H), 2.93-2.66 (m, 4H), 2.46-2.36 (m, 1H), 2.27-2.13 (m, 2H), 2.08-1.94 (m, 4H), 1.91-1.51 (m, 12H), 1.35-1.01 (m, 5H), 0.64-0.52 (m, 4H). ¹⁹F NMR (376 MHz, METHANOL-d4) δ=−120.71.

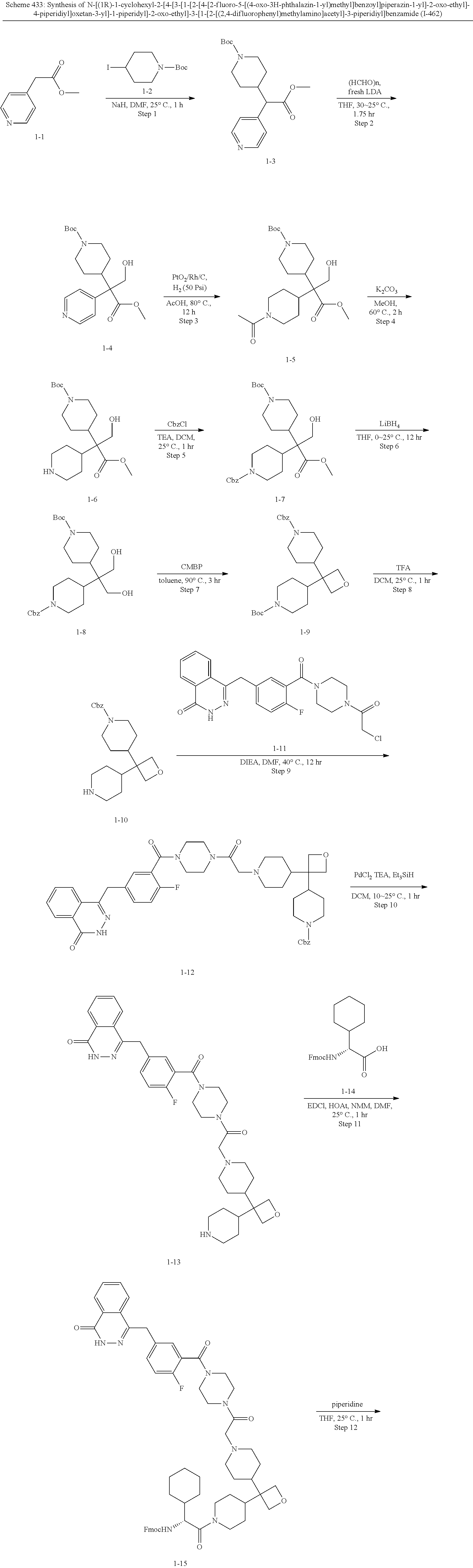

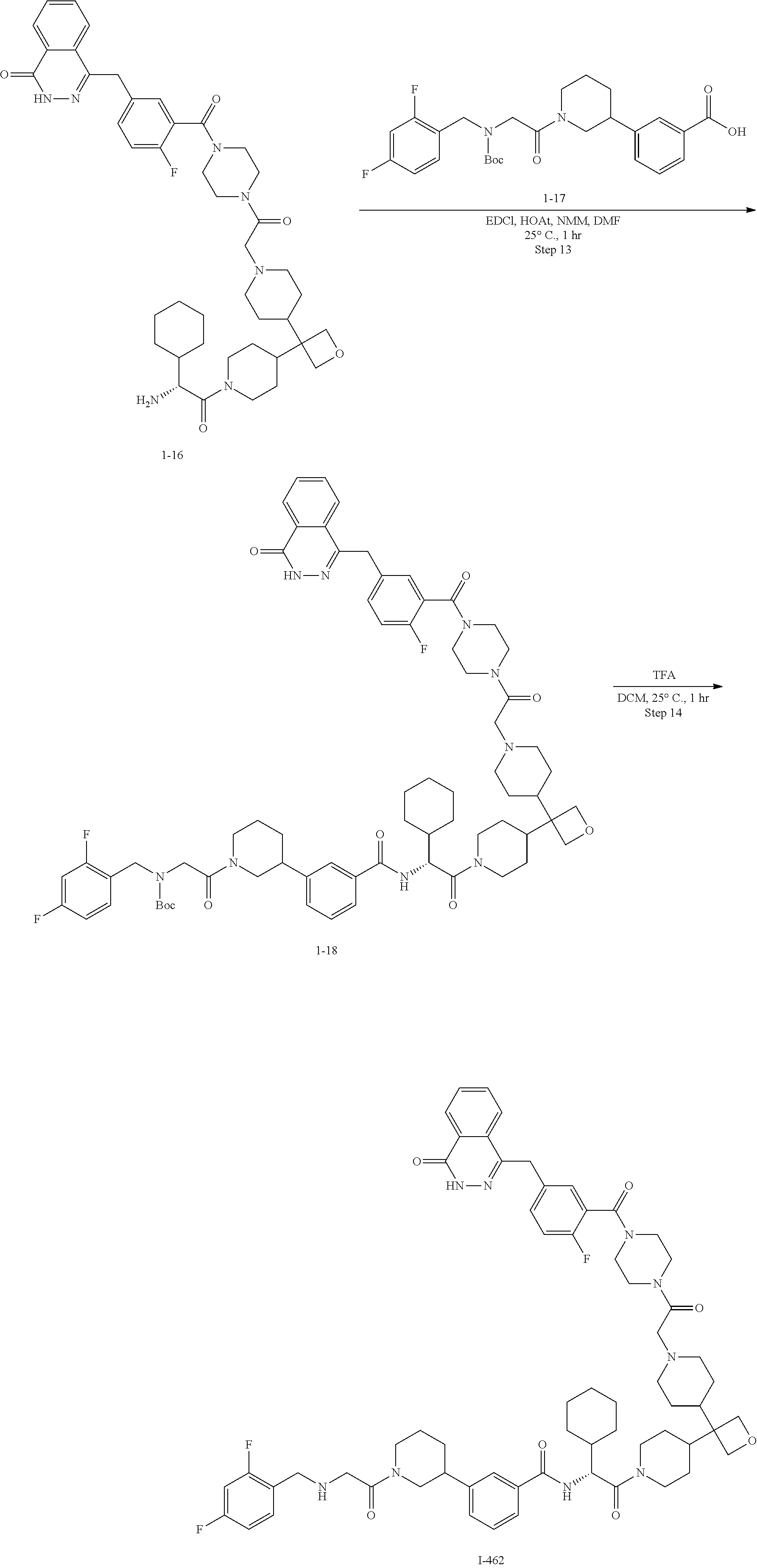 [SMILES: N[C@@H](C(=O)N1CCC(C2(C3CCN(CC(=O)N4CCN(C(=O)c5cc(Cc6n[nH]c(=O)c7ccccc67)ccc5F)CC4)CC3)COC2)CC1)C1CCCCC1 ; CC(C)(C)OC(=O)N(CC(=O)N1CCCC(c2cccc(C(=O)O)c2)C1)Cc1ccc(F)cc1F ; CC(C)(C)OC(=O)N(CC(=O)N1CCCC(c2cccc(C(=O)N[C@@H](C(=O)N3CCC(C4(C5CCN(CC(=O)N6CCN(C(=O)c7cc(Cc8n[nH]c(=O)c9ccccc89)ccc7F)CC6)CC5)COC4)CC3)C3CCCCC3)c2)C1)Cc1ccc(F)cc1F ; O=C(N[C@@H](C(=O)N1CCC(C2(C3CCN(CC(=O)N4CCN(C(=O)c5cc(Cc6n[nH]c(=O)c7ccccc67)ccc5F)CC4)CC3)COC2)CC1)C1CCCCC1)c1cccc(C2CCCN(C(=O)CNCc3ccc(F)cc3F)C2)c1]

### Step 1: Synthesis of Intermediate 1-3

To a solution of Intermediate 1-1 (10 g, 66.15 mmol, 1 eq) in DMF (100 mL) was added NaH (3.97 g, 99.23 mmol, 60% purity, 1.5 eq) and the mixture was stirred at 25° C. for 0.5 hr under N₂. Then to the mixture was added Intermediate 1-2 (20.58 g, 66.15 mmol, 1 eq) and it was stirred at 25° C. for 0.5 hr. The reaction mixture was quenched by aq·NH₄Cl (100 mL). The reaction mixture was extracted with EA (100 mL*2). The combined organic layers were washed with brine (50 mL*1), dried over Na₂SO₄, filtered and concentrated under reduced pressure to give a residue. The residue was purified by flash silica gel chromatography (ISCO; 12 g SepaFlash Silica Flash Column, Eluent of 0˜8% Methanol/Dichloromethane gradient @80 mL/min). The eluent was concentrated under reduced pressure to give Intermediate 1-3 (10.5 g, 31.40 mmol, 47.46% yield, 100% purity) as a yellow solid. LCMS (Method D): Rt: 0.292 min, (M+H)=335.1. ¹H NMR (400 MHz, METHANOL-d4) δ=8.49 (d, J=6.0 Hz, 2H), 7.44 (d, J=6.0 Hz, 2H), 4.14-4.05 (m, 1H), 4.02-3.92 (m, 1H), 3.68 (s, 3H), 3.51-3.42 (m, 1H), 2.85-2.59 (m, 2H), 2.30-2.17 (m, 1H), 1.81-1.71 (m, 1H), 1.43 (s, 9H), 1.31-1.17 (m, 2H), 1.08-0.94 (m, 1H).

### Step 2: Synthesis of Intermediate 1-4

To a solution of N-isopropylpropan-2-amine (3.63 g, 35.88 mmol, 5.07 mL, 1.2 eq) in THF (30 mL) was added n-BuLi (2.5 M, 14.35 mL, 1.2 eq) at −30° C., the mixture was stirred at −30° C. for 15 min, then Intermediate 1-3 (10 g, 29.90 mmol, 1 eq) in THF (100 mL) was added to the mixture at 25° C., and the mixture was stirred at 25° C. for 30 min, followed by addition of (HCHO)n (1.80 g, 1.65 mL) at 25° C., and the mixture was stirred at 25° C. for 60 min under N₂ atmosphere. The reaction mixture was quenched by aq·NH₄Cl (100 mL) and extracted with EA (100 mL*2). The combined organic layers were dried over Na₂SO₄, filtered and concentrated under reduced pressure to give a residue. The residue was purified by flash silica gel chromatography (ISCO; 200 g SepaFlash Silica Flash Column, Eluent of 0˜15% MeOH/CH₂Cl₂ gradient @100 mL/min). The eluent was concentrated under reduced pressure to give Intermediate 1-4 (6.5 g, crude) as a white solid. LCMS (Method D): Rt: 0.269 min, (M+H)=365.1.

### Step 3: Synthesis of Intermediate 1-5

To a solution of Intermediate 1-4 (2 g, 5.49 mmol, 1 eq) in AcOH (20 mL) was added PtO₂ (1.00 g, 4.40 mmol, 8.02e−1 eq) and Rh/C (1 g, 5% purity). The flask was degassed and purged with H₂ 3 times, then the mixture was stirred at 80° C. for 12 hr under H₂ (50 psi) atmosphere. The crude reaction mixture was combined with another lot of material for workup. The mixture reaction was filtered and the filter cake was washed with MeOH (5 mL*3), then the filtrate was concentrated under reduced pressure to give Intermediate 1-5 (4.6 g, crude) as a yellow oil. LCMS (Method D): Rt: 0.304 min, (M+H)=413.2.

### Step 4: Synthesis of Intermediate 1-6

To a solution of Intermediate 1-5 (4.6 g, 11.15 mmol, 1 eq) in MeOH (50 mL) was added K₂CO₃ (3.08 g, 22.30 mmol, 2 eq). The mixture was stirred at 60° C. for 2 hr. The mixture was filtered and concentrated under vacuum. The residue was purified by prep-HPLC (column: Phenomenex luna C18 (250*70 mm, 10 um); mobile phase: [water(FA)-ACN]; gradient: 0%-300% B over 25 min). The eluent was concentrated under reduced pressure to remove ACN and then lyophilized to give Intermediate 1-6 (500 mg, 1.20 mmol, 10.77% yield, FA salt) as a white solid. LCMS (Method D): Rt: 0.269 min, (M+H)=371.2. ¹H NMR (400 MHz, METHANOL-d4) δ=8.53 (s, 1H), 4.19-4.07 (m, 2H), 3.89 (s, 2H), 3.68 (s, 3H), 3.46-3.36 (m, 2H), 3.02-2.91 (m, 2H), 2.75-2.60 (m, 2H), 2.35-2.23 (m, 1H), 2.14-2.03 (m, 1H), 2.02-1.85 (m, 2H), 1.81-1.64 (m, 3H), 1.54-1.47 (m, 2H), 1.45 (s, 9H), 1.38-1.24 (m, 1H).

### Step 5: Synthesis of Intermediate 1-7

To a solution of Intermediate 1-6 (500 mg, 1.20 mmol, 1 eq, FA salt) in CH₂Cl₂ (5 mL) was added TEA (364.42 mg, 3.60 mmol, 501.26 μL, 3 eq) and CbzCl (307.18 mg, 1.80 mmol, 257.06 μL, 1.5 eq). The mixture was stirred at 25° C. for 1 hr. The mixture was concentrated in vacuum. The crude product was purified by reversed-phase HPLC (0.1% FA condition). The eluent was concentrated under reduced pressure to remove ACN and then lyophilized to give Intermediate 1-7 (320 mg, 604.98 μmol, 50.40% yield, 95.4% purity) as a white solid. LCMS (Method D): Rt: 0.512 min, (M+Na)=527.2.

### Step 6: Synthesis of Intermediate 1-8

To a solution of Intermediate 1-7 (320 mg, 634.15 μmol, 1 eq) in THF (4 mL) was degassed and purged with N₂ 3 times. Then LiBH4 (2 M, 1.44 mL, 4.8% purity, 4.55 eq) was added into the mixture at 0° C. The mixture was stirred at 25° C. for 1 hr under N₂. The mixture was stirred at 25° C. for 11 hr under N₂. The reaction mixture was quenched by addition of saturated NH₄Cl (3 mL) at 25° C. The reaction mixture diluted with H₂O (1 mL) and extracted with EA (3 mL*2), The combined organic layers were dried over Na₂SO₄, filtered and concentrated under reduced pressure to give Intermediate 1-8 (300 mg, 614.35 μmol, 96.88% yield, 97.6% purity) as a yellow solid, which was used in the next step without further purification. LCMS (Method D): Rt: 0.448 min, (M+Na)=499.3.

### Step 7: Synthesis of Intermediate 1-9

To a solution of Intermediate 1-8 (300 mg, 629.45 μmol, 1 eq) in toluene (3 mL) was added 2-(tributyl-phosphanylidene) acetonitrile (379.80 mg, 1.57 mmol, 2.5 eq). The mixture was stirred at 90° C. for 3 hr. The mixture was concentrated in vacuum. The crude product was purified by reversed-phase HPLC (0.1% FA condition). The eluent was concentrated under reduced pressure to remove ACN and then lyophilized to give Intermediate 1-9 (200 mg, 399.92 μmol, 63.53% yield, 91.7% purity) as a yellow solid. LCMS (Method D): Rt: 0.488 min, (M+Na)=481.1.

### Step 8: Synthesis of Intermediate 1-10

A solution of Intermediate 1-9 (200 mg, 436.12 μmol, 1 eq) in CH₂Cl₂ (2 mL) and TFA (0.2 mL) was stirred at 25° C. for 1 hr. The mixture was diluted with aq·NaHCO₃ (2 mL) and extracted with CH₂Cl₂ (1 mL*2). The combined organic layers were dried over Na₂SO₄, filtered and concentrated under reduced pressure to give Intermediate 1-10 (180 mg, crude) as a yellow oil which was used in the next step without further purification. LCMS (Method D): Rt: 0.295 min, (M+H)=359.2.

### Step 9: Synthesis of Intermediate 1-12

To a solution of Intermediate 1-10 (180 mg, 502.13 μmol, 1 eq) and Intermediate 1-11 (222.38 mg, 502.13 μmol, 1 eq) in DMF (2 mL) was added DIEA (194.69 mg, 1.51 mmol, 262.38 μL, 3 eq). The mixture was stirred at 40° C. for 12 hr. The mixture was filtered. The filtrate was purified by reversed-phase HPLC (0.10% FA condition). The eluent was concentrated under reduced pressure to remove ACN and then lyophilized to give Intermediate 1-12 (250 mg, 256.81 μmol, 51.14% yield, 83.3% purity, FA salt) as a white solid. LCMS (Method D): Rt: 0.361 min, (M+H)=765.3. ¹H NMR (400 MHz, METHANOL-d4) δ=8.46 (s, 1H), 8.42-8.37 (m, 1H), 8.00-7.94 (m, 1H), 7.93-7.82 (m, 2H), 7.57-7.48 (m, 1H), 7.44-7.29 (m, 6H), 7.24-7.14 (m, 1H), 5.14 (s, 2H), 4.53-4.43 (m, 4H), 4.41 (s, 2H), 4.33-4.24 (m, 2H), 3.86-3.75 (m, 2H), 3.74-3.64 (m, 3H), 3.62-3.49 (m, 3H), 3.41-3.35 (m, 2H), 3.31-3.25 (m, 2H), 2.94-2.76 (m, 2H), 2.63-2.38 (m, 2H), 1.96-1.77 (m, 5H), 1.74-1.59 (m, 3H), 1.39-1.31 (m, 2H).

### Step 10: Synthesis of Intermediate 1-13

To a solution of Intermediate 1-12 (220 mg, 271.30 μmol, 1 eq, FA salt) in CH₂Cl₂ (2 mL) was added PdCl₂ (14.43 mg, 81.39 μmol, 0.3 eq) and TEA (54.91 mg, 542.60 μmol, 75.52 μL, 2 eq). The reaction mixture was degassed with N₂. Then a solution of Et₃SiH (126.18 mg, 1.09 mmol, 173.33 μL, 4 eq) in CH₂Cl₂ (1 mL) was added dropwise at 10° C. The reaction mixture was stirred at 25° C. for 1 hr. The mixture was filtered and the filtrate was concentrated in vacuum. Then the mixture diluted with NaHCO₃ (3 mL) and extracted with CH₂Cl₂ (3 mL*2). The combined organic layers were dried over Na₂SO₄, filtered and concentrated under reduced pressure to give a residue. The crude product was purified by reversed-phase HPLC (0.1% NH₃·H₂O condition). The eluent was concentrated under reduced pressure to remove ACN and then lyophilized to give Intermediate 1-13 (150 mg, 216.65 μmol, 79.85% yield, 91.1% purity) as a white solid. LCMS (Method D): Rt: 0.243 min, (M+H)=631.3.

### Step 11: Synthesis of Intermediate 1-15

To a solution of Intermediate 1-14 (78.21 mg, 206.10 μmol, 78.21 μL, 1 eq) in DMF (2 mL) was added EDCI (118.53 mg, 618.31 μmol, 3 eq), HOAt (28.05 mg, 206.10 μmol, 28.83 μL, 1 eq), Intermediate 1-13 (130 mg, 206.10 μmol, 1 eq) and NMM (104.23 mg, 1.03 mmol, 113.30 μL, 5 eq). The mixture was stirred at 25° C. for 1 hr. The mixture was filtered. The filtrate was purified by reversed-phase HPLC (0.1% FA condition). The eluent was concentrated under reduced pressure to remove ACN and then lyophilized to give Intermediate 1-15 (180 mg, 172.53 μmol, 83.71% yield, 95.1% purity) as a white solid. LCMS (Method D): Rt: 0.434 min, (M+H)=992.3.

### Step 12: Synthesis of Intermediate 1-16

To a solution of Intermediate 1-15 (150 mg, 151.18 μmol, 1 eq) in THF (1.6 mL) was added piperidine (172.44 mg, 2.03 mmol, 0.2 mL, 13.40 eq). The mixture was stirred at 25° C. for 1 hr. The mixture was filtered. The filtrate was purified by reversed-phase HPLC (0.1% NH₃·H₂O condition). The eluent was concentrated under reduced pressure to remove ACN and then lyophilized to give Intermediate 1-16 (100 mg, 128.84 μmol, 85.22% yield, 99.2% purity) was obtained as a white solid. LCMS (Method D): Rt: 0.287 min, (M+H)=770.3. ¹H NMR (400 MHz, METHANOL-d4) δ=8.39 (d, J=7.2 Hz, 1H), 8.01-7.95 (m, 1H), 7.93-7.82 (m, 2H), 7.55-7.48 (m, 1H), 7.43-7.36 (m, 1H), 7.23-7.15 (m, 1H), 4.75-4.66 (m, 1H), 4.55-4.44 (m, 3H), 4.41 (s, 2H), 4.21-4.10 (m, 1H), 3.85-3.48 (m, 7H), 3.41-3.36 (m, 1H), 3.30-3.21 (m, 3H), 3.19-2.96 (m, 4H), 2.73-2.60 (m, 1H), 2.17-2.02 (m, 2H), 2.01-1.74 (m, 8H), 1.73-1.46 (m, 7H), 1.38-1.03 (m, 6H). SFC: Rt: 1.031 min. ¹⁹F NMR (376 MHz, METHANOL-d4) δ=−120.802.

### Step 13: Synthesis of Intermediate 1-18

To a solution of Intermediate 1-17 (31.72 mg, 64.94 μmol, 1 eq) in DMF (1 mL) was added EDCI (37.35 mg, 194.82 μmol, 3 eq), HOAt (8.84 mg, 64.94 μmol, 9.08 μL, 1 eq), Intermediate 1-16 (50 mg, 64.94 μmol, 1 eq) and NMM (32.84 mg, 324.70 μmol, 35.70 μL, 5 eq). The mixture was stirred at 25° C. for 1 hr. The mixture was filtered. The filter liquor was purified by reversed-phase HPLC (0.1% NH₃·H₂O condition). The eluent was concentrated under reduced pressure to remove ACN and then lyophilized to give Intermediate 1-18 (60 mg, 46.92 μmol, 72.25% yield, 97.0% purity) as a white solid. LCMS (Method D): Rt: 0.429 min, (M+H)=1240.6.

### Step 14: Synthesis of I-462

A solution of Intermediate 1-18 (60 mg, 48.37 μmol, 1 eq) in CH₂Cl₂ (0.6 mL) and TFA (0.06 mL) was stirred at 25° C. for 1 hr. To the mixture was added aq·NaHCO₃ to adjust pH to 8. The mixture was filtered. The filtrate was purified by reversed-phase HPLC (0.1% NH₃·H₂O condition). The eluent was concentrated under reduced pressure to remove ACN and then lyophilized to give I-462 (37.54 mg, 32.85 μmol, 67.92% yield, 99.8% purity) as a white solid. LCMS (Method D): Rt: 0.361 min, (M+H)=1140.4. H NMR (400 MHz, METHANOL-d4) δ=8.40-8.34 (m, 1H), 7.98-7.92 (m, 1H), 7.91-7.80 (m, 2H), 7.76 (s, 1H), 7.73-7.68 (m, 1H), 7.51-7.36 (m, 5H), 7.20-7.13 (m, 1H), 7.00-6.88 (m, 2H), 5.00-4.92 (m, 1H), 4.72-4.64 (m, 1H), 4.61-4.49 (m, 2H), 4.48-4.37 (m, 6H), 3.90-3.78 (m, 4H), 3.74 (s, 2H), 3.70-3.65 (m, 1H), 3.57 (s, 1H), 3.55-3.42 (m, 3H), 3.39-3.34 (m, 1H), 3.29-3.24 (m, 2H), 3.23-3.10 (m, 3H), 3.08-2.97 (m, 2H), 2.86-2.63 (m, 3H), 2.11-2.00 (m, 3H), 1.97-1.73 (m, 11H), 1.72-1.64 (m, 2H), 1.63-1.40 (m, 5H), 1.36-1.21 (m, 4H), 1.18-1.06 (m, 2H). SFC: Rt: 6.546 min, 7.499 min. ¹⁹F NMR (376 MHz, METHANOL-d4) δ=−113.386, −116.253, −120.779.

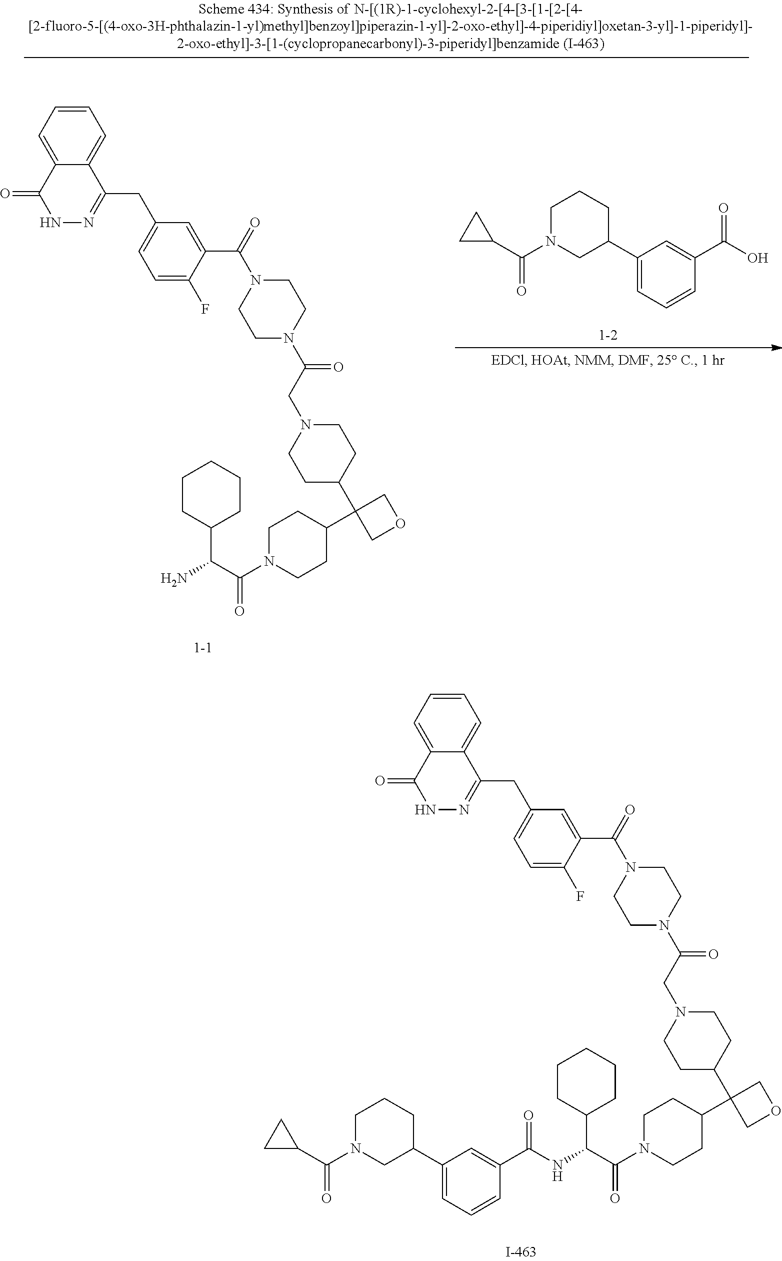 [SMILES: N[C@@H](C(=O)N1CCC(C2(C3CCN(CC(=O)N4CCN(C(=O)c5cc(Cc6n[nH]c(=O)c7ccccc67)ccc5F)CC4)CC3)COC2)CC1)C1CCCCC1 ; O=C(O)c1cccc(C2CCCN(C(=O)C3CC3)C2)c1 ; O=C(N[C@@H](C(=O)N1CCC(C2(C3CCN(CC(=O)N4CCN(C(=O)c5cc(Cc6n[nH]c(=O)c7ccccc67)ccc5F)CC4)CC3)COC2)CC1)C1CCCCC1)c1cccc(C2CCCN(C(=O)C3CC3)C2)c1]

To a solution of Intermediate 1-2 (14.20 mg, 51.95 μmol, 1 eq) in DMF (0.5 mL) was added EDCI (29.88 mg, 155.86 μmol, 3 eq), HOAt (7.07 mg, 51.95 μmol, 7.27 μL, 1 eq), Intermediate 1-1 (40 mg, 51.95 μmol, 1 eq) and NMM (26.27 mg, 259.76 μmol, 28.56 μL, 5 eq). The mixture was stirred at 25° C. for 1 hr. The reaction mixture was purified by reversed-phase column chromatography (0.1% FA condition). The eluent was concentrated under reduced pressure to remove ACN and then lyophilized to give I-463 (20.12 mg, 19.13 μmol, 36.83% yield, 97.5% purity) as a white solid. LCMS (Method D): Rt: 0.407 min, [M+H]⁺=1025.3. ¹H NMR (400 MHz, METHANOL-d4) δ=8.37 (d, J=7.6 Hz, 1H), 7.98-7.92 (m, 1H), 7.91-7.67 (m, 4H), 7.54-7.34 (m, 4H), 7.20-7.13 (m, 1H), 4.99-4.90 (m, 1H), 4.72-4.65 (m, 1H), 4.60-4.53 (m, 1H), 4.52-4.40 (m, 5H), 4.39 (s, 3H), 3.84-3.73 (m, 2H), 3.68 (s, 2H), 3.53 (s, 3H), 3.44 (s, 1H), 3.37 (s, 1H), 3.27 (s, 1H), 3.25-3.10 (m, 4H), 2.88-2.64 (m, 3H), 2.43-2.22 (m, 2H), 2.08-1.71 (m, 14H), 1.70-1.46 (m, 6H), 1.39-1.20 (m, 4H), 1.19-1.05 (m, 2H), 0.91-0.75 (m, 4H). ¹⁹F NMR (377 MHz, METHANOL-d4) δ=−120.749. SFC: Rt=5.144 min, 6.539 min

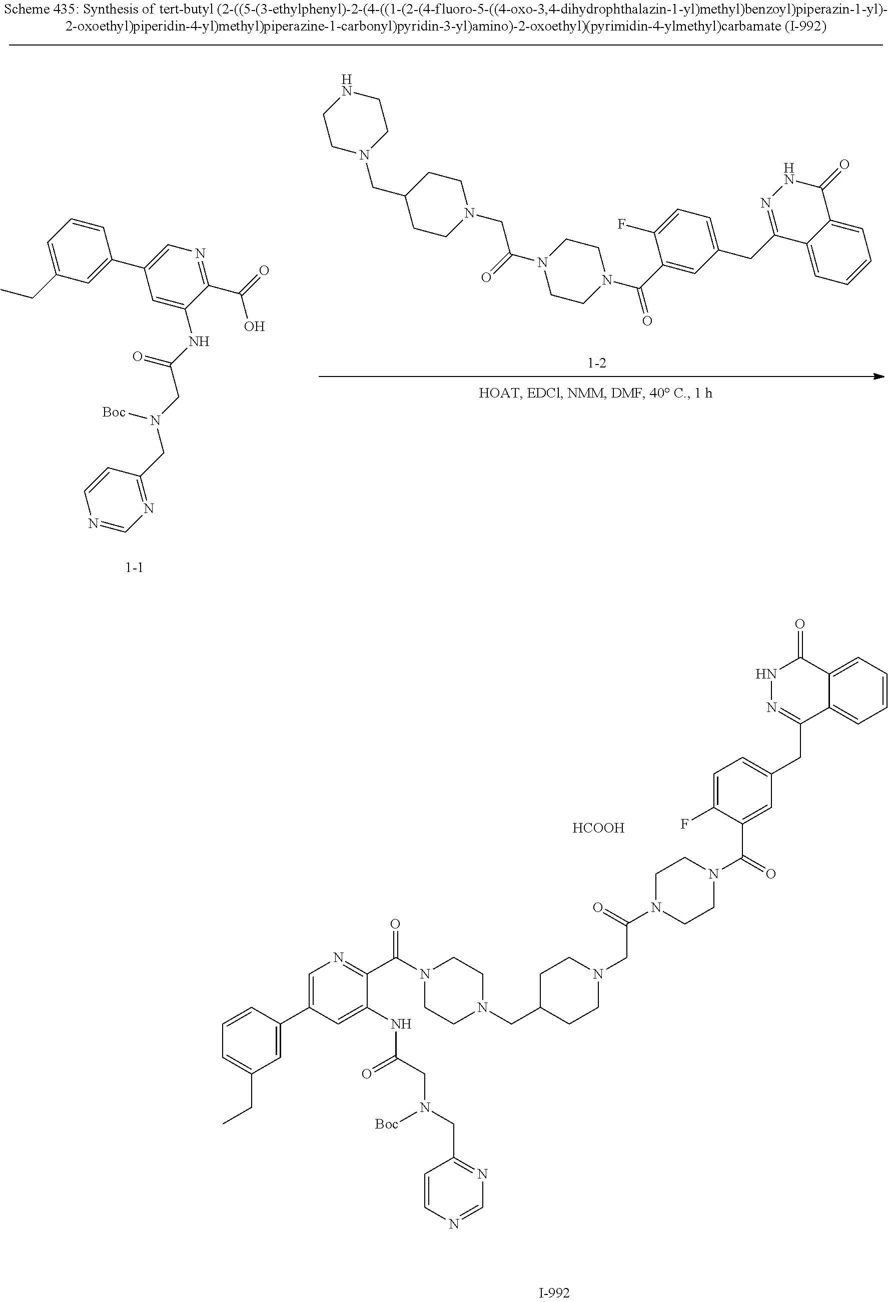 [SMILES: CCc1cccc(-c2cnc(C(=O)O)c(NC(=O)CN(Cc3ccncn3)C(=O)OC(C)(C)C)c2)c1 ; O=C(CN1CCC(CN2CCNCC2)CC1)N1CCN(C(=O)c2cc(Cc3n[nH]c(=O)c4ccccc34)ccc2F)CC1 ; CCc1cccc(-c2cnc(C(=O)N3CCN(CC4CCN(CC(=O)N5CCN(C(=O)c6cc(Cc7n[nH]c(=O)c8ccccc78)ccc6F)CC5)CC4)CC3)c(NC(=O)CN(Cc3ccncn3)C(=O)OC(C)(C)C)c2)c1 ; "HCOOH"]

To a solution of intermediate 1-1 (4 g, 8.14 mmol, 1 eq) and intermediate 1-2 (4.3 g, 6.11 mmol, 7.51e−1 eq, TFA salt) in DMF (40 mL) was added HOAt (553.82 mg, 4.07 mmol, 569.19 μL, 0.5 eq), EDCI (3.12 g, 16.28 mmol, 2 eq) and NMM (4.12 g, 40.69 mmol, 4.47 mL, 5 eq). The mixture was stirred at 40° C. for 1 hr. The reaction was purified by prep-HPLC (column: Phenomenex luna C18 (250*70 mm, 10 um); mobile phase: [water (FA)-ACN]; gradient: 18%-48% B over 30 min) and lyophilized to give the product. I-992 (5 g, 4.41 mmol, 54.25% yield, 97.939% purity, FA salt) was obtained as a brown solid. LCMS (Method D): Rt=0.397 min, [M+H]⁺=1063.5. ¹H NMR (400 MHz, METHANOL-d4) δ=9.16-9.03 (m, 1H), 8.74 (s, 1H), 8.67 (s, 1H), 8.41-8.28 (m, 2H), 8.17 (s, 2H), 7.94 (d, J=4.0 Hz, 1H), 7.91-7.78 (m, 2H), 7.57 (d, J=4.8 Hz, 1H), 7.55-7.47 (m, 3H), 7.43 (t, J=7.6 Hz, 1H), 7.38 (s, 1H), 7.32 (d, J=7.2 Hz, 1H), 7.17 (t, J=8.8 Hz, 1H), 4.70 (s, 2H), 4.38 (s, 2H), 4.30-4.23 (m, 2H), 3.82 (s, 2H), 3.56 (d, J=3.6 Hz, 4H), 3.41-3.33 (m, 2H), 3.24-3.18 (m, 4H), 3.16-2.98 (m, 2H), 2.77-2.72 (m, 3H), 2.72-2.64 (m, 3H), 2.58-2.45 (m, 2H), 2.15-1.94 (m, 3H), 1.67-1.52 (m, 2H), 1.47 (s, 4H), 1.35-1.25 (m, 12H). ¹⁹F NMR (377 MHz, METHANOL-d4) δ=−120.659.

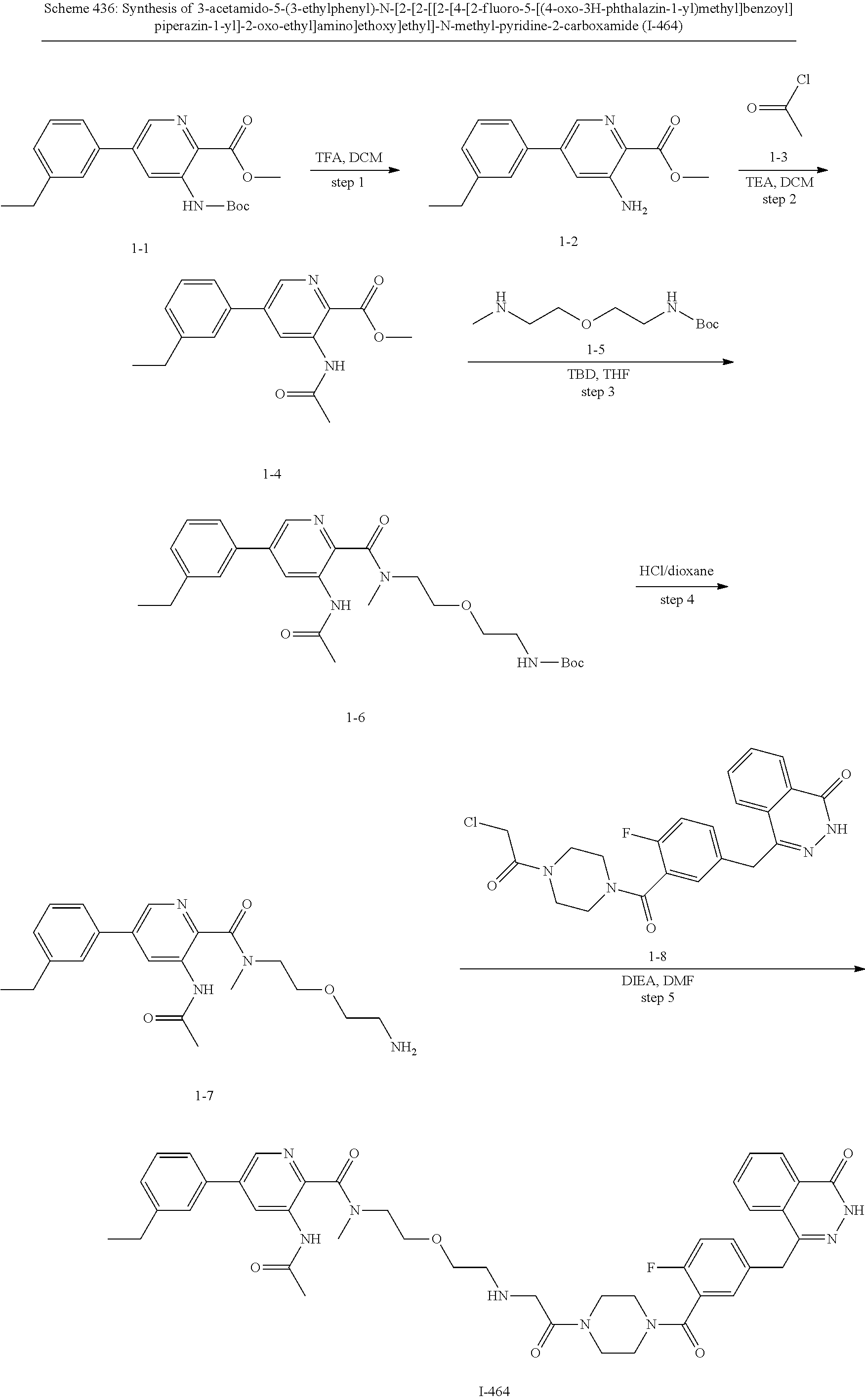

### Step 1: Synthesis of Intermediate 1-2

To a solution of Intermediate 1-1 (400 mg, 1.12 mmol, 1 eq) in DCM (3 mL) was added TFA (1.54 g, 13.46 mmol, 1 mL, 12.00 eq). The mixture was stirred at 25° C. for 1 hr. The reaction mixture was concentrated under reduced pressure to give the crude product. The crude product was used in the next step without further purification. Intermediate 1-2 (570 mg, crude, TFA salt) was obtained as a yellow solid. LCMS (Method E): Rt=0.473 min, [M+H]⁺=257.1.

### Step 2: Synthesis of Intermediate 1-4

To a solution of Intermediate 1-2 (570 mg, 1.54 mmol, 1 eq, TFA salt) and Intermediate 1-3 (483.29 mg, 6.16 mmol, 437.77 μL, 4 eq) in DCM (6 mL) was added TEA (778.75 mg, 7.70 mmol, 1.07 mL, 5 eq). The mixture was stirred at 25° C. for 1 hr. The reaction was diluted with H₂O (3 mL), and extracted with ethyl acetate (3 mL*3). The organic phase was washed with saturated aqueous NaCl (5 mL), dried over Na₂SO₄, filtered and concentrated under reduced pressure to give a residue. The residue was purified by flash silica gel chromatography (ISCO; 20 g SepaFlash Silica Flash Column, Eluent of 0˜100% Ethyl acetate/Petroleum ether gradient @20 mL/min). The eluent was concentrated to afford the desired product. Intermediate 1-4 (300 mg, 955.20 μmol, 62.06% yield, 94.99% purity) as white gum which. LCMS (Method E): Rt=0.552 min, [M+H]⁺=299.3.

### Step 3: Synthesis of Intermediate 1-6

To a solution of Intermediate 1-4 (200 mg, 670.39 μmol, 1 eq) and Intermediate 1-5 (175.61 mg, 804.46 μmol, 1.2 eq) in THF (2 mL) was added 3,4,6,7,8,9-hexahydro-2H-pyrimido[1,2-a]pyrimidine (102.65 mg, 737.42 μmol, 1.1 eq). The mixture was stirred at 80° C. for 1 hr. The reaction was diluted with H₂O (3 mL), and extracted with ethyl acetate (3 mL*3). The organic phase was washed with saturated aqueous NaCl (5 mL). Then dried over Na₂SO₄, filtered and concentrated under reduced pressure to give a residue. The residue was purified by flash silica gel chromatography (ISCO; 20 g SepaFlash Silica Flash Column, Eluent of 0-100% Ethyl acetate/Petroleum ether gradient @20 mL/min). The eluent was concentrated to afford the desired product. Intermediate 1-6 (200 mg, 379.71 μmol, 56.64% yield, 92% purity) was obtained as a white solid. LCMS (Method E): Rt=0.542 min, [M+H]⁺=485.3. ¹H NMR (400 MHz, DMSO-d₆) δ=9.82-9.57 (m, 1H), 8.64 (d, J=12.4 Hz, 1H), 8.42-8.18 (m, 1H), 7.58-7.49 (m, 2H), 7.43-7.40 (m, 1H), 7.31 (d, J=7.2 Hz, 1H), 6.87-6.70 (m, 1H), 3.64-3.56 (m, 3H), 3.13-3.00 (m, 4H), 2.74-2.67 (m, 2H), 2.07 (d, J=9.2 Hz, 3H), 1.39-1.32 (m, 11H), 1.24-1.20 (m, 4H).

### Step 4: Synthesis of Intermediate 1-7

A solution of Intermediate 1-6 (100 mg, 206.36 μmol, 1 eq) in HCl/dioxane (2 M, 1.00 mL, 9.69 eq) was stirred at 25° C. for 1 hr. The reaction mixture was concentrated under reduced pressure to give the crude product. The crude product was used in the next step without further purification. Intermediate 1-7 (86 mg, 202.59 μmol, 98.17% yield, 99.16% purity, HCl salt) was obtained as a yellow solid. LCMS (Method E): Rt=0.416 min, [M+H]⁺=385.2.

### Step 5: Synthesis of I-464

To a solution of Intermediate 1-7 (86 mg, 204.31 μmol, 1 eq, HCl salt) and Intermediate 1-8 (72.39 mg, 163.45 μmol, 0.8 eq) in DMF (1 mL) was added DIEA (132.03 mg, 1.02 mmol, 177.93 μL, 5 eq). The mixture was stirred at 40° C. for 1 hr. The reaction mixture was concentrated to give the crude product. The product was purified by prep-HPLC (column: YMC-Actus Triart C18 150*30 mm*7 um; mobile phase: [water (FA)-ACN]; gradient: 20%-50% B over 10 min) and the eluent was concentrated to remove MeCN and then lyophilized. I-464 (24.51 mg, 27.28 μmol, 13.35% yield, 93.15% purity, FA salt) was obtained as a white solid. LCMS (Method E): Rt=0.460 min, [M+H]⁺=791.6. ¹H NMR (400 MHz, METHANOL-d₄) δ=8.68-8.58 (m, 1H), 8.56-8.24 (m, 2H), 8.21 (s, 1H), 7.99-7.78 (m, 3H), 7.58-7.21 (m, 6H), 7.20-7.06 (m, 1H), 4.36 (d, J=12.4 Hz, 2H), 4.00-3.86 (m, 1H), 3.85-3.41 (m, 12H), 3.31-3.06 (m, 7H), 3.03-2.92 (m, 1H), 2.81-2.64 (m, 2H), 2.23-2.11 (m, 3H), 1.33-1.20 (m, 3H).

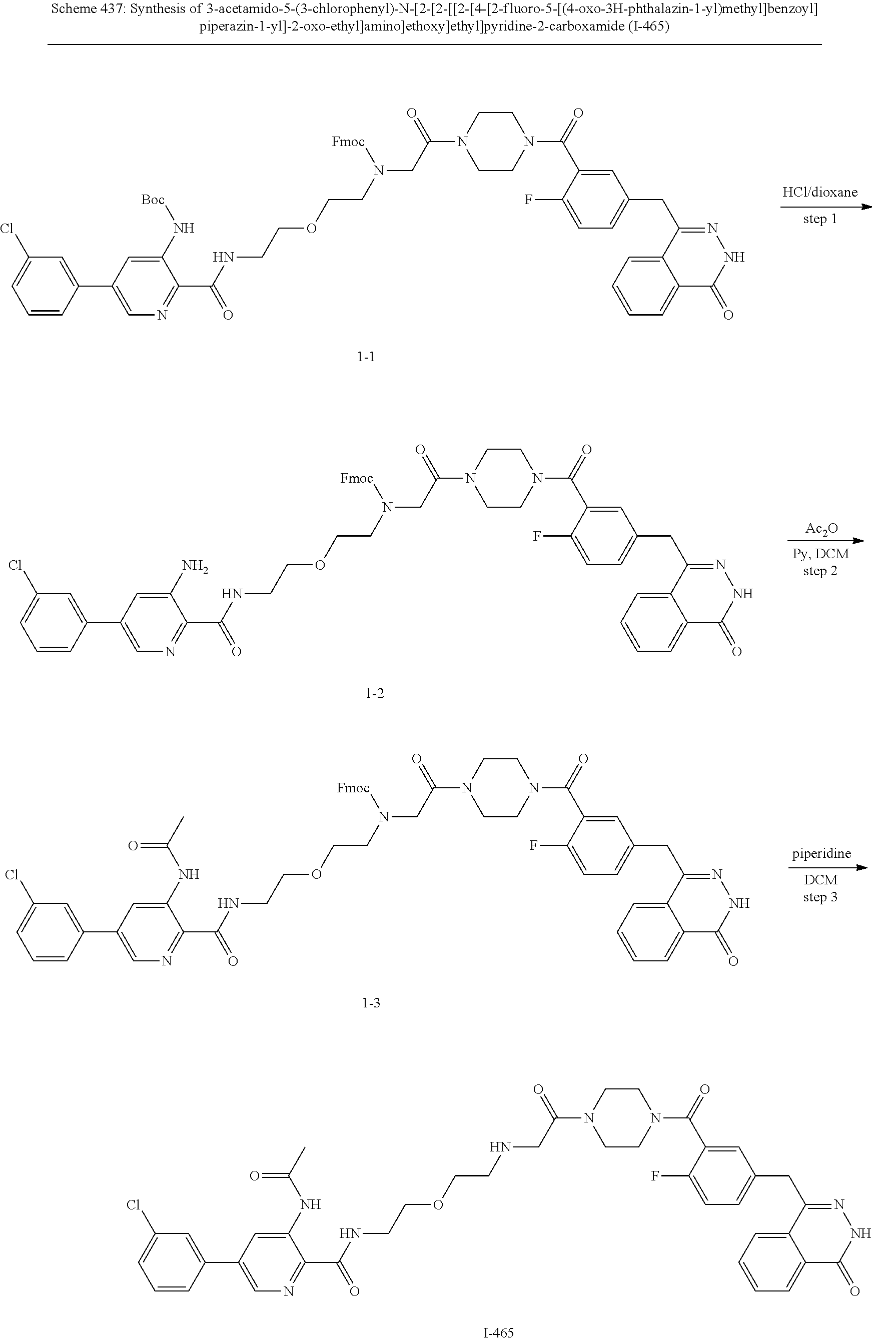

### Step 1: Synthesis of Intermediate 1-2

A solution of Intermediate 1-1 (500 mg, 470.12 μmol, 1 eq) in HCl/dioxane (5 mL) was stirred at 25° C. for 1 hr. The reaction mixture was concentrated to give the crude product. The crude product was triturated with EA (100 mL) at 25° C. for 30 min to give Intermediate 1-2 (450 mg, 369.48 μmol, 78.59% yield, 82.1% purity, HCl salt) as an off-white solid. LCMS (Method E): Rt=0.589 min, [M+H]⁺=963.3.

### Step 2: Synthesis of Intermediate 1-3

To a solution of Intermediate 1-2 (200 mg, 200.02 μmol, 1 eq, HCl salt) and pyridine (47.46 mg, 600.06 μmol, 48.43 μL, 3 eq) in DCM (2 mL) was added acetyl chloride (14.13 mg, 180.02 μmol, 12.80 L, 0.9 eq) at 0° C. Then the mixture was stirred at 0° C. for 1 hr. The reaction mixture was poured into water (30 mL) and extracted with DCM (30 mL*2), the organic layer was washed with brine (30 mL) and dried over Na₂SO₄. The organic phase was concentrated to give the crude product. The crude product was triturated with PE:EA=3:1 (5 mL) at 25° C. for 30 min to give Intermediate 1-3 (160 mg, 137.65 mol, 68.82% yield, 86.5% purity) as a white solid. LCMS (Method E): Rt=0.630 min, [M+H]⁺=1005.5.

### Step 3: Synthesis of I-465

To a solution of Intermediate 1-3 (160 mg, 159.13 μmol, 1 eq) in DCM (2 mL) was added piperidine (67.75 mg, 795.64 μmol, 78.57 μL, 5 eq). Then the mixture was stirred at 25° C. for 1 hr. The reaction mixture was filtered to give the filtrate. The filtrate was purified by reverse phase column (HCl condition) to give I-465 (26.98 mg, 32.74 μmol, 20.58% yield, 99.48% purity, HCl salt) as a yellow solid. LCMS (Method E): Rt=0.476 min, [M+H]⁺=783.3. ¹H NMR (400 MHz, DMSO-d₆) δ=12.63 (br s, 1H), 12.10 (br s, 1H), 9.31-8.98 (m, 4H), 8.64 (br d, J=1.6 Hz, 1H), 8.26 (br d, J=7.6 Hz, 1H), 8.00-7.75 (m, 4H), 7.73-7.64 (m, 1H), 7.57 (br s, 2H), 7.47-7.23 (m, 3H), 4.33 (br s, 2H), 4.19-4.00 (m, 2H), 3.82-3.36 (m, 12H), 3.28-3.11 (m, 4H), 2.19 (br d, J=2.8 Hz, 3H).

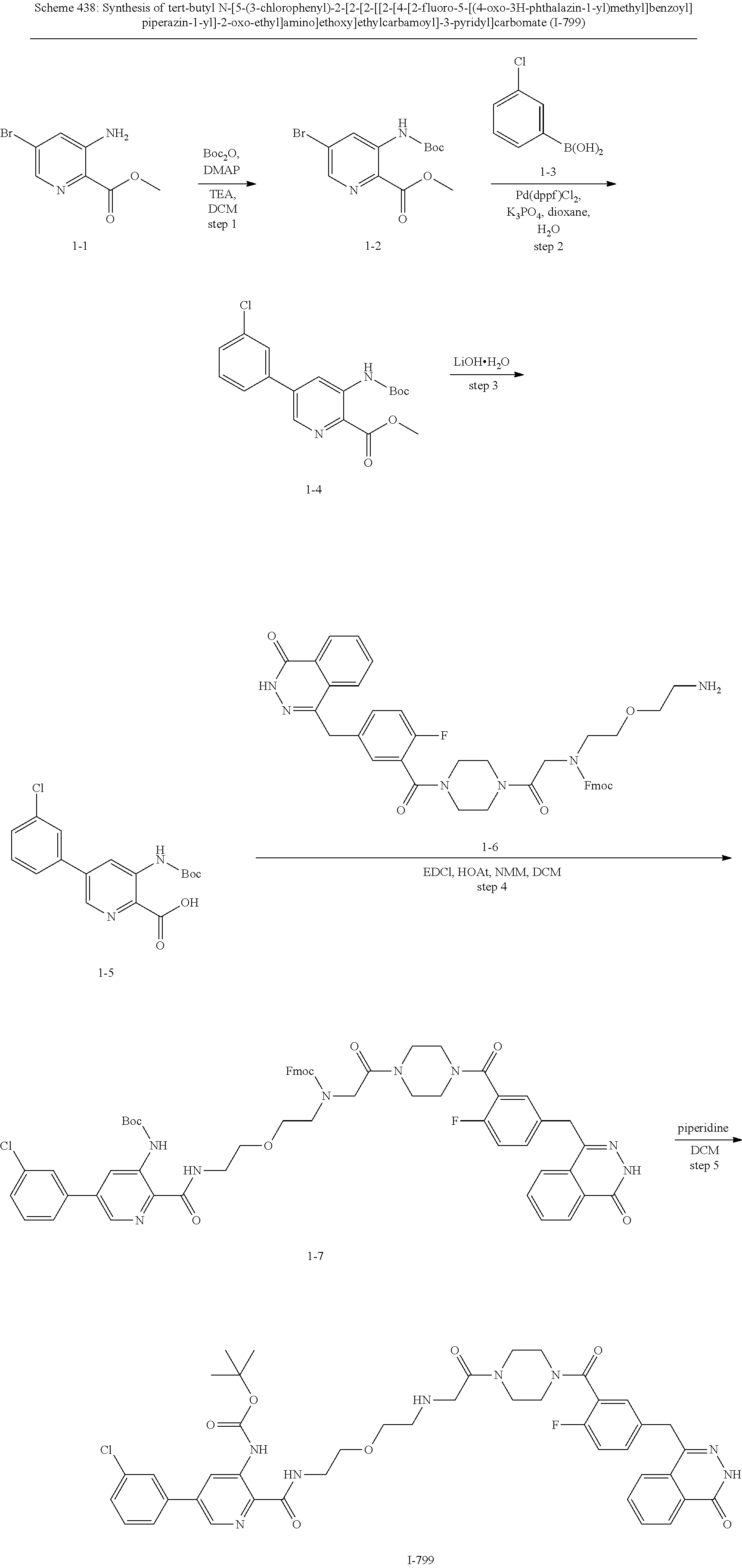

### Step 1: Synthesis of Intermediate 1-2

To a solution of Intermediate 1-1 (3 g, 12.98 mmol, 1 eq), TEA (6.57 g, 64.92 mmol, 9.04 mL, 5 eq) and DMAP (158.63 mg, 1.30 mmol, 0.1 eq) in DCM (20 mL) was added Boc₂O (5.67 g, 25.97 mmol, 5.97 mL, 2 eq). Then the mixture was stirred at 25° C. for 1 hr. The reaction mixture was poured into water (50 mL) and extracted with DCM (50 mL*2), the organic layer was washed with brine (50 mL) and dried over Na₂SO₄, and concentrated to give the crude product. The crude product was purified by flash silica gel chromatography (SiO₂, PE:EA=10:1 to 3:1) to give Intermediate 1-2 (4.2 g, 9.74 mmol, 75.00% yield, 100% purity) as a white solid. LCMS (Method E): Rt=0.565 min, [M+H]⁺=331.0.

### Step 2: Synthesis of Intermediate 1-4

To a solution of Intermediate 1-2 (4 g, 9.27 mmol, 1 eq), Intermediate 1-3 (1.45 g, 9.27 mmol, 1 eq), and ditert-butyl(cyclopenta-1,4-dien-1-yl)phosphane; dichloropalladium;iron (604.48 mg, 927.48 μmol, 0.1 eq) in dioxane (40 mL) and H₂O (4 mL) was added K₃PO₄ (5.91 g, 27.82 mmol, 3 eq). Then the mixture was stirred at 40° C. for 1 hr under N₂. The reaction mixture was poured into water (100 mL) and extracted with EA (100 mL*2), the organic layer was washed with brine (100 mL) and dried over Na₂SO₄, concentrated to give the crude product. The crude product was purified by flash silica gel chromatography (SiO₂, PE:EA=3:1 to 1:1) to give Intermediate 1-4 (4.2 g, 8.79 mmol, 94.79% yield, 96.9% purity) as a white solid. LCMS (Method E): Rt=0.617 min, [M+H]⁺=363.2. ¹H NMR (400 MHz, CHLOROFORM-d) δ=8.88 (d, J=1.6 Hz, 1H), 7.78 (d, J=1.6 Hz, 1H), 7.62 (s, 1H), 7.56-7.43 (m, 3H), 4.00 (s, 3H), 1.44 (s, 18H).

### Step 3: Synthesis of Intermediate 1-5

To a solution of Intermediate 1-4 (4.1 g, 8.86 mmol, 1 eq) in MeOH (40 mL) and H₂O (40 mL) was added LiOH·H₂O (1.86 g, 44.28 mmol, 5 eq). Then the mixture was stirred at 25° C. for 1 hr. The reaction mixture was poured into water (100 mL) and adjusted to pH=2 with 1N HCl, and extracted with EA (100 mL*2). The organic layer was washed with brine (100 mL) and dried over Na₂SO₄, and concentrated to give the crude product. The crude product was used for the next step without further purification. Intermediate 1-5 (2.5 g, 7.09 mmol, 80.04% yield, 98.9% purity) was obtained as a white solid. LCMS (Method E): Rt=0.585 min, [M+H]⁺=349.2. ¹H NMR (400 MHz, DMSO-d₆) δ=10.58 (br s, 1H), 8.94 (s, 1H), 8.69 (s, 1H), 7.87 (s, 1H), 7.76 (br d, J=6.4 Hz, 1H), 7.69-7.57 (m, 2H), 1.55 (s, 8H).

### Step 4: Synthesis of Intermediate 1-7

To a solution of Intermediate 1-5 (1 g, 2.87 mmol, 1 eq), EDCI (1.10 g, 5.73 mmol, 2 eq), HOAt (195.12 mg, 1.43 mmol, 200.54 μL, 0.5 eq), and NMM (1.45 g, 14.34 mmol, 1.58 mL, 5 eq) in DCM (20 mL) was added Intermediate 1-6 (2.21 g, 2.87 mmol, 1 eq, HCl salt). Then the mixture was stirred at 25° C. for 1 hr. The reaction mixture was poured into water (30 mL) and extracted with DCM (30 mL*2), the organic layer was washed with brine (30 mL) and dried over Na₂SO₄. The mixture was concentrated to give the crude product. The crude product was purified by flash silica gel chromatography (SiO₂, PE:EA=10:1 to 1:1) to give Intermediate 1-7 (2.2 g, 1.94 mmol, 67.53% yield, 93.6% purity) as a white solid. LCMS (Method E): Rt=0.698 min, [M−99]⁺=963.3.

### Step 5: Synthesis of I-799

To a solution of Intermediate 1-7 (500 mg, 470.12 μmol, 1 eq) in DCM (5 mL) was added piperidine (200.15 mg, 2.35 mmol, 232.13 μL, 5 eq). Then the mixture was stirred at 25° C. for 1 hr. The reaction mixture was filtered to give the filtrate. The filtrate was purified by reverse phase chromatography (HCl condition) to give I-799 (380 mg, 424.68 μmol, 90.34% yield, 98.1% purity, HCl salt) as a white solid. LCMS (Method E): Rt=0.537 min, [M+H]⁺=841.2. ¹H NMR (400 MHz, DMSO-d₆) δ=12.61 (s, 1H), 11.51 (d, J=2.8 Hz, 1H), 9.20 (br d, J=6.0 Hz, 1H), 9.04-8.89 (m, 3H), 8.60-8.58 (m, 1H), 8.42-8.21 (m, 1H), 8.00-7.79 (m, 4H), 7.76-7.67 (m, 1H), 7.64-7.54 (m, 2H), 7.52-7.41 (m, 1H), 7.40-7.31 (m, 1H), 7.26-7.24 (m, 1H), 4.34 (br s, 2H), 4.20-4.00 (m, 2H), 3.75 (br s, 2H), 3.65-3.11 (m, 14H), 1.50 (s, 9H).

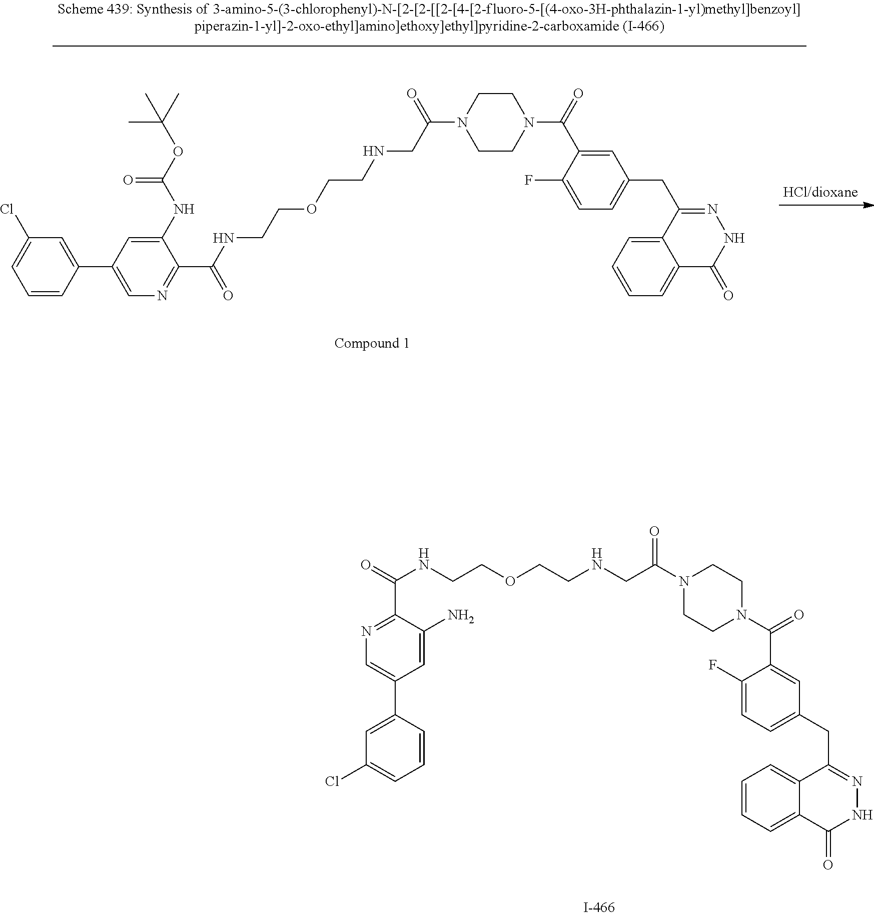 [SMILES: CC(C)(C)OC(=O)Nc1cc(-c2cccc(Cl)c2)cnc1C(=O)NCCOCCNCC(=O)N1CCN(C(=O)c2cc(Cc3n[nH]c(=O)c4ccccc34)ccc2F)CC1 ; Nc1cc(-c2cccc(Cl)c2)cnc1C(=O)NCCOCCNCC(=O)N1CCN(C(=O)c2cc(Cc3n[nH]c(=O)c4ccccc34)ccc2F)CC1]

A solution of I-466 (200 mg, 237.72 μmol, 1 eq) in HCl/dioxane (2 mL) was stirred at 25° C. for 1 hr. The reaction mixture was filtered to give the filtrate. The filtrate was purified by reverse phase chromatography (HCl salt) to give I-466 (49.71 mg, 63.92 μmol, 26.89% yield, 100% purity, HCl salt) as a yellow solid by lyophilization. LCMS (Method E): Rt=0.467 min, [M+H]⁺=741.3. ¹H NMR (400 MHz, DMSO-d₆) δ=12.62 (s, 1H), 9.08 (br d, J=3.6 Hz, 2H), 8.67 (br d, J=6.0 Hz, 1H), 8.26 (d, J=8.0 Hz, 1H), 8.17-8.08 (m, 1H), 8.00-7.80 (m, 3H), 7.72 (s, 1H), 7.64 (br d, J=6.4 Hz, 1H), 7.58-7.44 (m, 4H), 7.37-7.35 (m, 1H), 7.29-7.17 (m, 1H), 4.33 (s, 2H), 4.20-4.03 (m, 2H), 3.76 (br s, 2H), 3.70-3.54 (m, 5H), 3.52-3.40 (m, 4H), 3.35-3.09 (m, 5H).

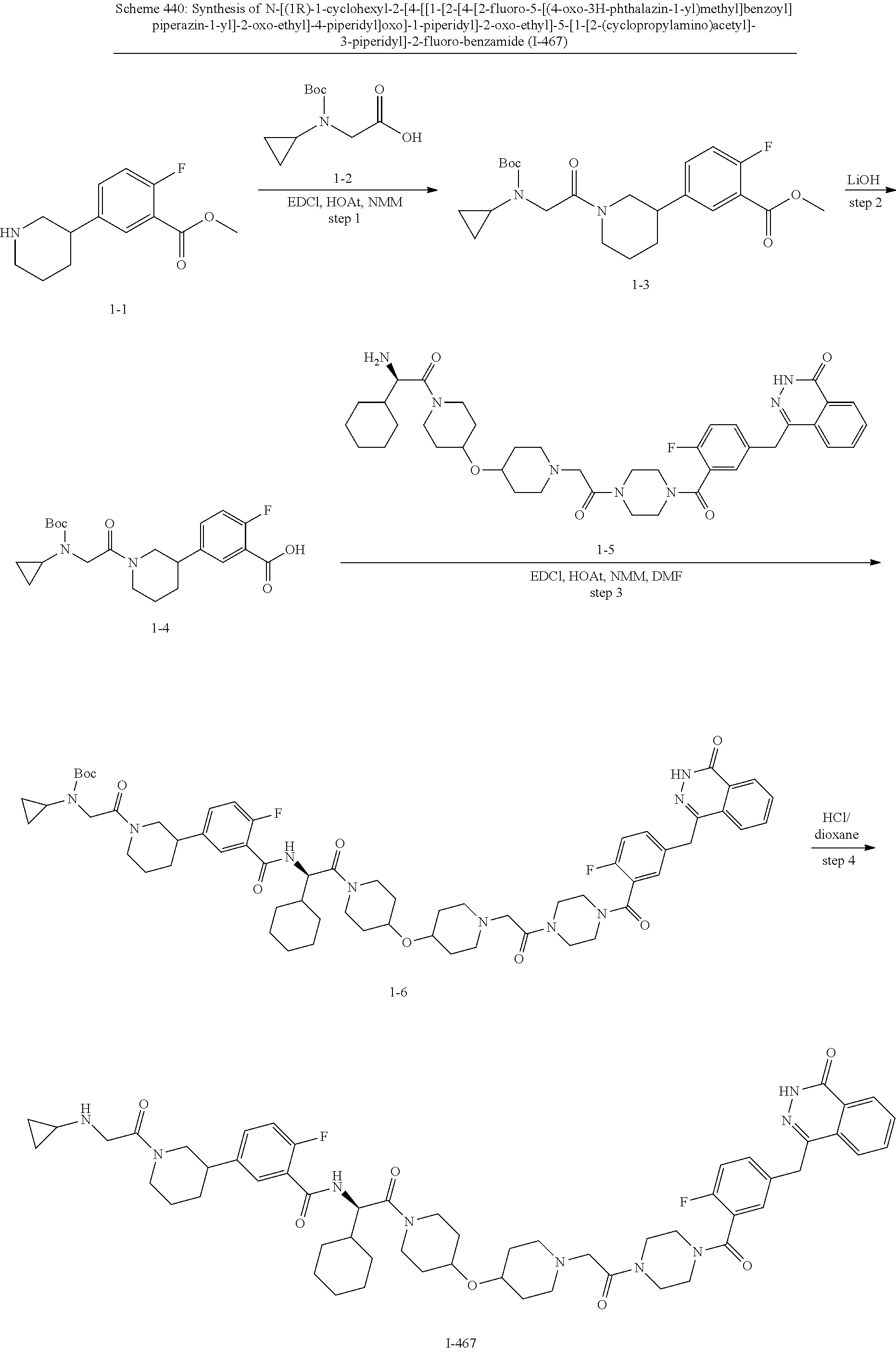

### Step 1: Synthesis of Intermediate 1-3

To a solution of Intermediate 1-1 (400 mg, 1.69 mmol, 1 eq), EDCI (646.36 mg, 3.37 mmol, 2 eq), HOAt (229.46 mg, 1.69 mmol, 235.83 μL, 1 eq) and NMM (852.59 mg, 8.43 mmol, 926.73 μL, 5 eq) in DMF (4 mL) was added Intermediate 1-2 (362.87 mg, 1.69 mmol, 1 eq). The mixture was stirred at 25° C. for 2 hr. The mixture was diluted with water (5 ml), and extracted with EtOAc (3 ml*3). The organic layer was concentrated under vacuum. This residue used in the next step without purification. Intermediate 1-3 (750 mg, crude) was obtained as a light yellow oil. LCMS (Method E): Rt=0.563 min, [M-Boc+H]⁺=335.1.

### Step 2: Synthesis of Intermediate 1-4

To a solution of Intermediate 1-3 (750 mg, 1.73 mmol, 1 eq) in THF (2.5 mL), MeOH (2.5 mL) and H₂O (2.5 mL) was added LiOH·H₂O (144.87 mg, 3.45 mmol, 2 eq). The mixture was stirred at 25° C. for 1 hr. The mixture was concentrated under vacuum. The residue was adjusted to pH=4 with an HCl solution and extracted with EA. The organic layer was dried with anhydrous Na₂SO₄, filtered and concentrated under vacuum. This residue was used in the next step without purification. Intermediate 1-4 (790 mg, crude) was obtained as a light yellow oil.

### Step 3: Synthesis of Intermediate 1-6

To a solution of Intermediate 1-4 (790 mg, 1.88 mmol, 1 eq), EDCI (720.35 mg, 3.76 mmol, 2 eq), HOAt (255.73 mg, 1.88 mmol, 262.83 μL, 1 eq) and NMM (950.19 mg, 9.39 mmol, 1.03 mL, 5 eq) in DMF (7 mL) was added Intermediate 1-5 (1.37 g, 1.88 mmol, 1 eq). The mixture was stirred at 25° C. for 2 hr. The reaction mixture was triturated with water (5 ml), filtered and the filter cake was washed with water (10 mL*2), and the filter cake was dried under vacuum. The residue was purified by reversed-phase HPLC (0.1% NH₃·H₂O condition) and concentrated under vacuum to remove MeCN and dried by lyophilization. The residue was purified by Prep-HPLC (column: Phenomenex luna C18 150*40 mm*15 um; mobile phase: [water (FA)-ACN]; gradient: 28%-58% B over 15 min) and dried by lyophilization. Intermediate 1-6 (660 mg, 579.31 μmol, 30.83% yield, 99.39% purity) was obtained as a light yellow solid. LCMS (Method G): Rt=0.738 min, [M+H]⁺=1133.9. ¹H NMR (400 MHz, METHANOL-d₄) δ=8.26 (br d, J=8.0 Hz, 1H), 7.84 (br d, J=8.0 Hz, 1H), 7.80-7.69 (m, 2H), 7.65-7.57 (m, 1H), 7.42-7.29 (m, 3H), 7.13-7.02 (m, 2H), 5.41 (s, 2H), 4.94 (br s, 1H), 4.44 (br d, J=8.0 Hz, 1H), 4.28 (s, 2H), 4.15-4.07 (m, 1H), 3.93 (br d, J=16.0 Hz, 1H), 3.85-3.76 (m, 2H), 3.73-3.61 (m, 4H), 3.60-3.34 (m, 6H), 3.17 (br s, 2H), 3.10 (br d, J=4.0 Hz, 1H), 3.08-2.99 (m, 1H), 2.74-2.56 (m, 5H), 2.25-2.10 (m, 2H), 1.88-1.62 (m, 12H), 1.61-1.48 (m, 5H), 1.37 (br s, 11H), 1.20 (br s, 1H), 1.12-1.00 (m, 3H), 0.65-0.52 (m, 4H). ¹⁹F NMR (377 MHz, METHANOL-d₄) δ=−117.972, −118.210, −120.409.

### Step 4: Synthesis of I-467

To a solution of Intermediate 1-6 (600 mg, 529.88 μmol, 1 eq) in DCM (2 mL) was added HCl/dioxane (2 M, 4 mL, 15.10 eq). The mixture was stirred at 25° C. for 30 min. The reaction mixture was combined with another lot of material (50 mg of Intermediate 1-6 starting material) and for work up. The reaction mixture was concentrated under vacuum. The crude product was purified by reversed-phase HPLC (0.1% NH₃·H₂O condition) and concentrated under vacuum to remove MeCN and dried by lyophilization. The residue was purified by Prep-HPLC (column: Waters Xbridge Prep OBD C18 150*40 mm*10 um; mobile phase: [water (NH₄HCO₃)-ACN]; gradient: 28%-58% B over 15 min) and dried by lyophilization. The residue was purified by Prep-HPLC (column: Phenomenex luna C18 150*40 mm*15 um; mobile phase: [water (TFA)-ACN]; gradient: 20%-50% B over 15 min) and dried by lyophilization. I-467 (319.73 mg, 278.63 μmol, 52.58% yield, 99.89% purity, TFA salt) as a white solid was obtained. LCMS (Method G): Rt=0.645 min, [M+H]⁺=1032.8. SFC: Rt=5.065 min, 5.928 min. ¹H NMR (400 MHz, METHANOL-d₄) δ=8.36 (d, J=8.0 Hz, 1H), 7.97-7.92 (m, 1H), 7.90-7.81 (m, 2H), 7.73-7.63 (m, 1H), 7.55-7.45 (m, 2H), 7.41-7.35 (m, 1H), 7.24-7.14 (m, 2H), 5.02-4.96 (m, 1H), 4.58-4.47 (m, 1H), 4.38 (s, 2H), 4.34-4.16 (m, 4H), 4.05-3.98 (m, 1H), 3.97-3.90 (m, 1H), 3.86-3.69 (m, 6H), 3.65 (br d, J=4.0 Hz, 1H), 3.60-3.42 (m, 5H), 3.37 (br s, 4H), 3.25-3.07 (m, 2H), 2.88-2.71 (m, 3H), 2.15-1.96 (m, 5H), 1.92-1.64 (m, 12H), 1.57 (br s, 1H), 1.32-1.23 (m, 2H), 1.22-1.06 (m, 3H), 0.96-0.86 (m, 4H). ¹⁹F NMR (377 MHz, METHANOL-d₄) δ=−77.128, −118.199, −118.515, −120.702.

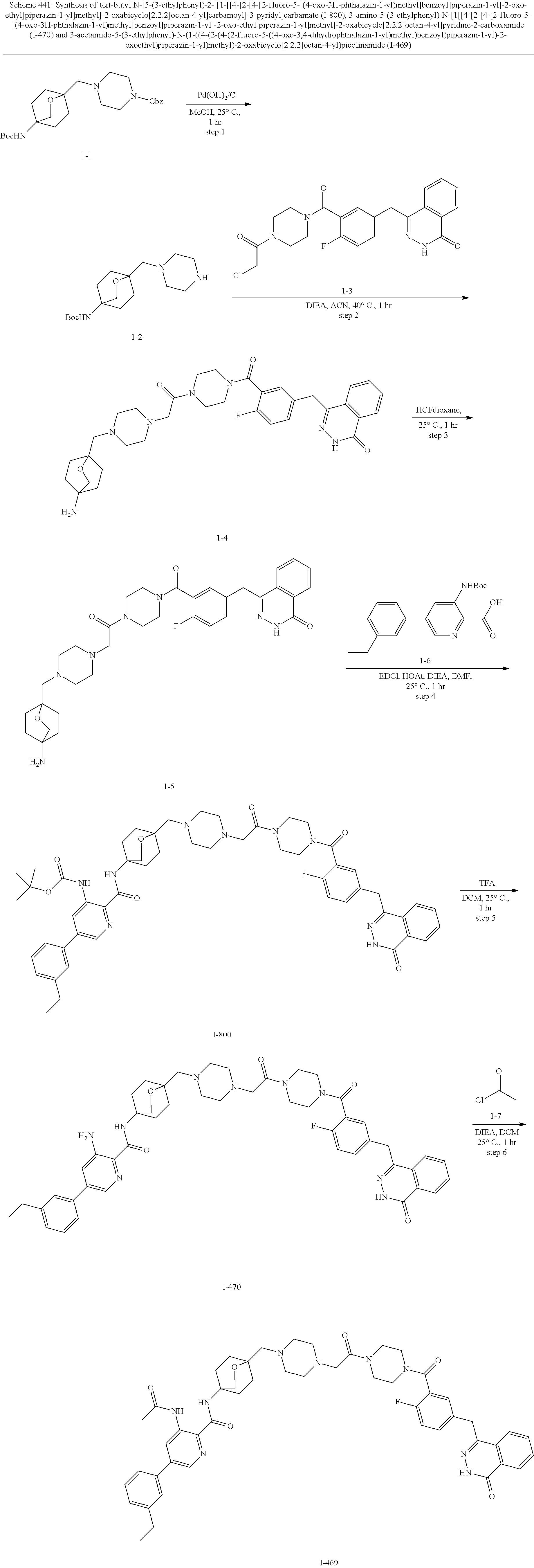

### Step 1: Synthesis of Intermediate 1-2

A mixture of intermediate 1-1 (800 mg, 1.74 mmol, 1 eq), Pd(OH)₂/C (200 mg, 20% purity) in MeOH (10 mL) was degassed and purged with N₂ 3 times, and then the mixture was degassed and purged with H₂ (15 psi) 3 times. The mixture was stirred at 25° C. for 1 hr under H₂ atmosphere (15 psi). The resulting mixture was filtered and washed with MeOH (10 ml*3). The filtrate was concentrated under reduced pressure give Intermediate 1-2 (510 mg, 1.57 mmol, 90.02% yield) as a white solid which was used in the next step without further purification. LCMS (Method D): Rt=0.207 min, [M+H]⁺=326.2.

### Step 2: Synthesis of Intermediate 1-4

To a solution of intermediate 1-2 (400 mg, 1.23 mmol, 1 eq) and intermediate 1-3 (598.76 mg, 1.35 mmol, 1.1 eq) in ACN (4 mL) was added DIEA (476.55 mg, 3.69 mmol, 642.25 μL, 3 eq). The mixture was stirred at 40° C. for 2 hr. The reaction mixture was concentrated under reduced pressure to give a residue. The residue was purified by flash silica gel chromatography (ISCO; 20 g SepaFlash Silica Flash Column, Eluent of 0˜75% Ethyl acetate/Methanol @40 mL/min). Intermediate 1-4 (680 mg, crude) was obtained as a white solid. LCMS (Method D): Rt=0.295 min, [M+H]⁺=732.4.

### Step 3: Synthesis of Intermediate 1-5

To a solution of intermediate 1-4 (630 mg, 860.83 μmol, 1 eq) was added HCl/dioxane (2 M, 7 mL, 16.26 eq). The mixture was stirred at 25° C. for 1 hr. The reaction mixture was concentrated under reduced pressure to give Intermediate 1-5 (440 mg, 696.49 μmol, 80.91% yield) as a white solid which was used in the next step without further purification. LCMS (Method D): Rt=0.211 min, [M+H]⁺=632.3.

### Step 4: Synthesis of I-800

To a solution of Intermediate 1-5 (390 mg, 617.34 μmol, 1 eq) and Intermediate 1-6 (422.74 mg, 1.23 mmol, 2 eq) in DMF (4 mL) was added EDCI (355.03 mg, 1.85 mmol, 3 eq), NMM (312.22 mg, 3.09 mmol, 339.37 μL, 5 eq) and HOAt (84.03 mg, 617.34 μmol, 86.36 μL, 1 eq). The mixture was stirred at 80° C. for 1 hr. The reaction mixture was purified by reversed phase chromatography (neutral condition) followed by lyophilization to give product. Then the product was repurified by reversed-phase chromatography (0.1% FA condition) followed by lyophilization to give I-800 (110 mg, 115.05 mol, 18.64% yield, 100% purity) as a white solid. LCMS (Method D): Rt=0.563 min, [M+H]⁺=956.5. ¹H NMR (400 MHz, METHANOL-d4) δ=8.99 (d, J=1.6 Hz, 1H), 8.44 (d, J=2.0 Hz, 1H), 8.37 (d, J=8.0 Hz, 1H), 7.99-7.92 (m, 1H), 7.92-7.79 (m, 2H), 7.55-7.46 (m, 3H), 7.46-7.35 (m, 2H), 7.31 (d, J=7.2 Hz, 1H), 7.17 (d, J=8.8 Hz, 1H), 4.39 (s, 2H), 4.12 (s, 2H), 3.82-3.65 (m, 4H), 3.58-3.48 (m, 2H), 3.35 (s, 2H), 3.24 (s, 1H), 2.75 (d, J=7.6 Hz, 2H), 2.71-2.47 (m, 8H), 2.38-2.26 (m, 4H), 2.10-2.02 (m, 4H), 1.81-1.71 (m, 2H), 1.56 (s, 9H), 1.29 (t, J=7.6 Hz, 4H). ¹⁹F NMR (377 MHz, METHANOL-d4) δ=−120.792.

### Step 5: Synthesis of I-470

To a solution of I-800 (35 mg, 36.61 μmol, 1 eq) in DCM (0.2 mL) was added TFA (79.82 mg, 700.00 μmol, 0.4 mL, 19.12 eq). The mixture was stirred at 25° C. for 1 hr. The reaction mixture was concentrated under reduced pressure to give a residue. The crude product was purified by reversed phase chromatography (0.1% TFA condition) followed by lyophilization to give product. I-470 (30 mg, 28.45 μmol, 77.73% yield, 92% purity, TFA salt) was obtained as a yellow solid. LCMS (Method D): Rt=0.460 min, [M+H]⁺=856.4. ¹H NMR (400 MHz, METHANOL-d4) δ=8.38 (d, J=7.2 Hz, 1H), 8.05 (d, J=2.0 Hz, 1H), 7.98-7.93 (m, 1H), 7.90-7.82 (m, 2H), 7.52 (d, J=6.0 Hz, 1H), 7.45 (s, 1H), 7.42-7.35 (m, 3H), 7.34 (d, J=2.0 Hz, 1H), 7.27 (d, J=7.2 Hz, 1H), 7.18 (t, J=8.8 Hz, 1H), 4.39 (s, 2H), 4.19 (s, 1H), 4.17-4.16 (m, 1H), 3.86-3.73 (m, 4H), 3.71 (d, J=4.4 Hz, 2H), 3.62 (d, J=6.4 Hz, 1H), 3.53 (d, J=5.2 Hz, 1H), 3.45 (s, 1H), 3.25 (s, 1H), 3.13-3.03 (m, 4H), 2.93 (d, J=10.0 Hz, 2H), 2.82-2.64 (m, 3H), 2.37 (d, J=2.8 Hz, 2H), 2.17-2.03 (m, 5H), 1.87-1.78 (m, 2H), 1.35-1.23 (m, 5H). ¹⁹F NMR (377 MHz, METHANOL-d4) δ=−77.317, −120.785.

### Step 6: Synthesis of I-469

To a solution of I-470 (40 mg, 46.73 μmol, 1 eq) and intermediate 1-7 (4.03 mg, 51.40 μmol, 3.65 L, 1.1 eq) in DCM (0.5 mL) was added DIEA (18.12 mg, 140.19 μmol, 24.42 μL, 3 eq). The mixture was stirred at 25° C. for 1 hr. The reaction mixture was concentrated under reduced pressure to give a residue. The crude product was purified by reversed-phase chromatography (0.1% TFA condition) followed by lyophilization to give product. I-469 (33 mg, 32.27 μmol, 69.06% yield, 98.967% purity, TFA salt) was obtained as a white solid. LCMS (Method D): Rt=0.399 min, [M+H]⁺=898.4. ¹H NMR (400 MHz, METHANOL-d4) δ=9.25 (d, J=2.0 Hz, 1H), 8.53 (d, J=2.0 Hz, 1H), 8.38 (d, J=7.6 Hz, 1H), 7.98-7.92 (m, 1H), 7.91-7.81 (m, 2H), 7.54-7.47 (m, 3H), 7.45-7.35 (m, 2H), 7.32 (d, J=7.2 Hz, 1H), 7.18 (t, J=9.2 Hz, 1H), 4.39 (s, 2H), 4.20 (s, 2H), 3.85-3.74 (m, 4H), 3.71 (d, J=5.2 Hz, 1H), 3.61 (s, 1H), 3.54 (s, 1H), 3.44 (d, J=3.6 Hz, 1H), 3.39 (s, 1H), 3.23-3.25 (m, 2H), 3.11 (d, J=1.6 Hz, 4H), 2.90 (s, 2H), 2.73-2.75 (m, 2H), 2.42-2.34 (m, 2H), 2.24 (s, 3H), 2.21-2.06 (m, 5H), 1.88-1.79 (m, 2H), 1.32-1.26 (m, 5H). ¹⁹F NMR (377 MHz, METHANOL-d4) δ=−77.287, −120.777.

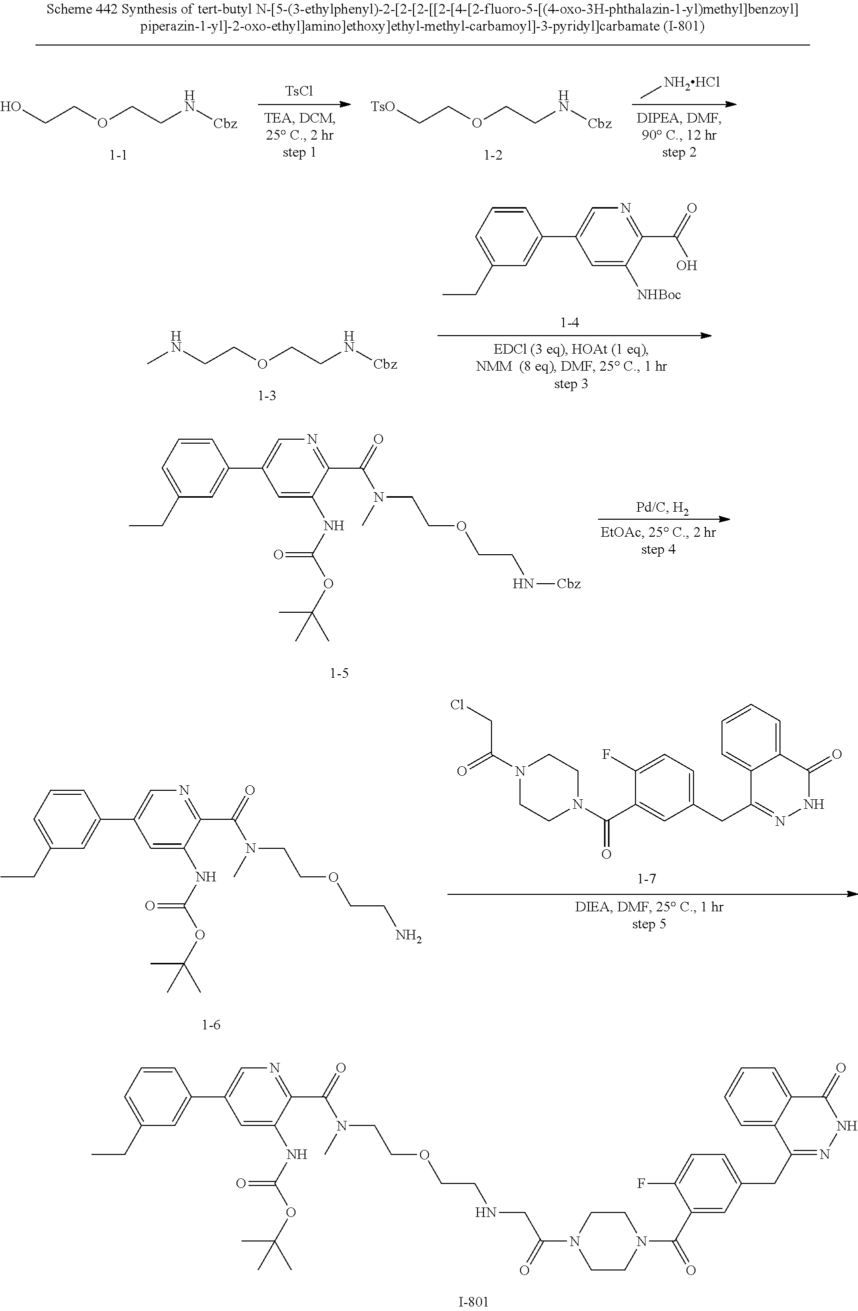

### Step 1: Synthesis of Intermediate 1-2

To a solution of Intermediate 1-1 (500 mg, 2.09 mmol, 1 eq) in DCM (5 mL) was added TEA (634.37 mg, 6.27 mmol, 872.58 μL, 3 eq) and 4-methylbenzenesulfonyl chloride (929.40 mg, 4.88 mmol, 2.33 eq). The mixture was stirred at 25° C. for 2 hr. The reaction mixture was quenched by addition of water (5 mL) at 25° C., and extracted with EA (5 mL*3). The combined organic layers were washed with NaCl solution (5 mL*3), dried over Na₂SO₄, filtered and concentrated under reduced pressure to give a residue. The residue was purified by flash silica gel chromatography (ISCO; 20 g SepaFlash Silica Flash Column, Eluent of 0˜100% Ethyl acetate/Petroleum ether gradient @30 mL/min). Intermediate 1-2 (750 mg, 1.89 mmol, 90.31% yield, 99% purity) was obtained as a colorless oil. LCMS (Method E): Rt=0.519 min, [M+Na]⁺=416.0.

### Step 2: Synthesis of Intermediate 1-3

To a solution of Intermediate 1-2 (700 mg, 1.78 mmol, 1 eq), methanamine; hydrochloride (1.44 g, 21.35 mmol, 12 eq) in DMF (10 mL) was added DIPEA (689.81 mg, 5.34 mmol, 929.67 μL, 3 eq). The mixture was stirred at 90° C. for 12 hr. The reaction mixture was quenched by addition of water (5 mL) at 25° C., and extracted with EA (5 mL*3). The combined organic layers were washed with a NaCl solution (5 mL*3), dried over Na₂SO₄, filtered and concentrated under reduced pressure to give a residue. The residue was purified by reversed phase chromatography (26.8*125 mm, 40 g of XB—C18, 20-40 m, 120 Å) Mobile phase: A for H₂O (0.1% FA v/v) and B for acetonitrile; Gradient: B 30%-50% in 30 min; Flow rate: 60 ml/min; Column temperature: R.T. Wavelength: 220 nm/254 nm). The eluent was concentrated to remove organic solvents. Intermediate 1-3 (270 mg, 877.50 μmol, 49.32% yield, 82% purity) was obtained as a white solid. LCMS (Method E): Rt=0.368 min, [M+H]⁺=253.1.

### Step 3: Synthesis of Intermediate 1-5

To a solution of Intermediate 1-3 (200 mg, 792.68 μmol, 1 eq) and Intermediate 1-4 (217.12 mg, 634.14 μmol, 0.8 eq) in DMF (2 mL) was added EDCI (455.87 mg, 2.38 mmol, 3 eq) and HOAt (107.89 mg, 792.68 μmol, 110.89 μL, 1 eq), NMM (641.42 mg, 6.34 mmol, 697.19 μL, 8 eq). The mixture was stirred at 25° C. for 1 hr. The reaction mixture was quenched by addition of water (5 mL) at 25° C., and then extracted with EA (5 mL*3). Then the organic layers were dried over Na₂SO₄, filtered and concentrated under reduced pressure to give a residue. The residue was purified by flash silica gel chromatography (ISCO; 40 g SepaFlash Silica Flash Column, Eluent of 0˜100% Ethyl acetate/Petroleum ether gradient @60 mL/min). Intermediate 1-5 (120 mg, 141.50 μmol, 17.85% yield, 68% purity) was obtained as a colorless oil. LCMS (Method E): Rt=0.636 min, [M+H]⁺=577.3.

### Step 4: Synthesis of Intermediate 1-6

To a mixture of Pd/C (46.13 mg, 43.35 μmol, 10% purity, 0.5 eq) in EtOAc (0.5 mL) was added Intermediate 1-5 (50 mg, 86.70 μmol, 1 eq) under N₂ atmosphere, and then the mixture was degassed and purged with H₂ 3 times. The mixture was stirred at 25° C. under H₂ (15 Psi) for 2 hr. The reaction mixture was filtered under N₂ atmosphere and washed with MeOH (5 mL*3), then concentrated under reduced pressure to give a residue. The crude product was used in the next step without further purification. Intermediate 1-6 (60 mg, crude) was obtained as a colorless oil.

LCMS (Method E): Rt=0.495 min, [M+H]⁺=443.2.

### Step 5: Synthesis of I-801

To a solution of Intermediate 1-6 (50 mg, 112.98 μmol, 1 eq) and Intermediate 1-7 (50.04 mg, 112.98 μmol, 1 eq) in DMF (0.5 mL) was added DIEA (43.81 mg, 338.94 μmol, 59.04 μL, 3 eq). The mixture was stirred at 25° C. for 1 hr. The reaction mixture was quenched by addition of water (5 mL) at 25° C., and extracted with EA (5 mL*3). The combined organic layers were washed with a NaCl solution (5 mL*3), dried over Na₂SO₄, filtered and concentrated under reduced pressure to give a residue. The product was purified by prep-HPLC (column: Phenomenex luna C18 150*25 mm*10 um; mobile phase: [water (HCl)-MeOH]; gradient: 25%-55% B over 9 min) and the eluent was concentrated to remove MeCN and then lyophilized. I-801 (21 mg, 24.24 μmol, 21.46% yield, 98% purity) was obtained as a white solid. LCMS (Method E): Rt=0.524 min, [M+H]⁺=849.6. SFC: Rt=1.587 min, ee %=93.804%. ¹H NMR (400 MHz, METHANOL-d₄) δ=8.58-8.41 (m, 2H), 8.36 (d, J=8.0 Hz, 1H), 7.99-7.90 (m, 1H), 7.89-7.78 (m, 2H), 7.56-7.43 (m, 3H), 7.43-7.22 (m, 3H), 7.20-7.06 (m, 1H), 4.36 (d, J=12.4 Hz, 2H), 3.87-3.63 (m, 8H), 3.62-3.34 (m, 6H), 3.27 (s, 1H), 3.23-3.08 (m, 4H), 3.01 (d, J=2.4 Hz, 1H), 2.82 (d, J=2.8 Hz, 1H), 2.77-2.65 (m, 2H), 1.52 (d, J=2.8 Hz, 9H), 1.31-1.22 (m, 3H). ¹⁹F NMR (377 MHz, METHANOL-d₄) δ=−120.64.

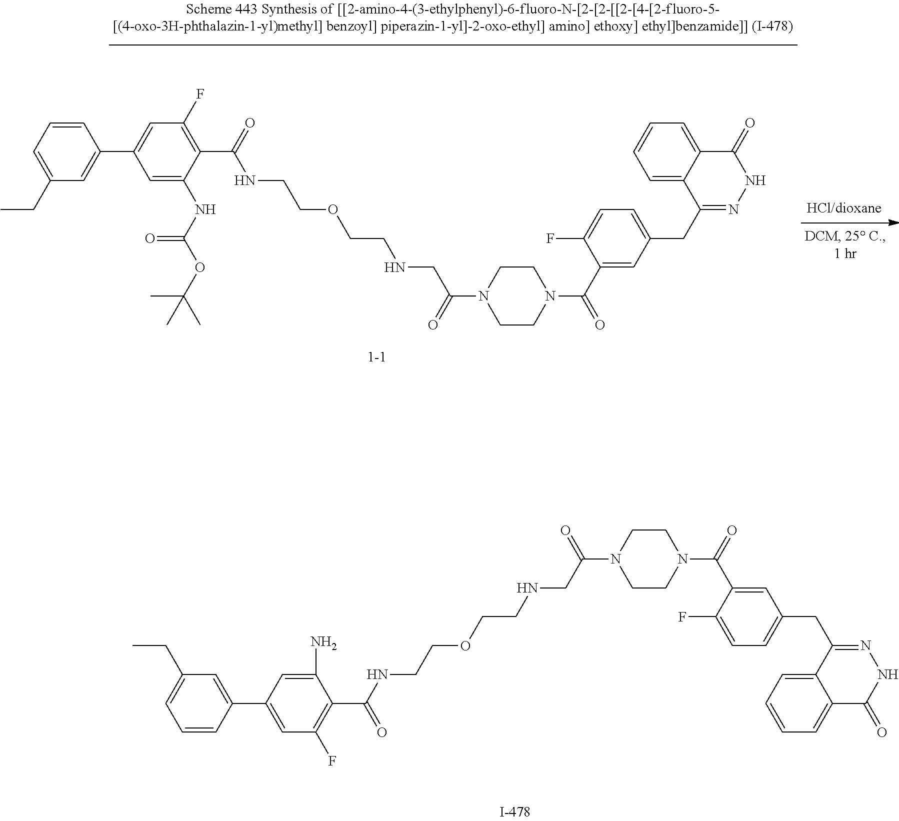 [SMILES: CCc1cccc(-c2cc(F)c(C(=O)NCCOCCNCC(=O)N3CCN(C(=O)c4cc(Cc5n[nH]c(=O)c6ccccc56)ccc4F)CC3)c(NC(=O)OC(C)(C)C)c2)c1 ; CCc1cccc(-c2cc(N)c(C(=O)NCCOCCNCC(=O)N3CCN(C(=O)c4cc(Cc5n[nH]c(=O)c6ccccc56)ccc4F)CC3)c(F)c2)c1]

To a solution of intermediate 1-1 (40 mg, 46.95 μmol, 1 eq) in DCM (0.1 mL) was added HCl/dioxane (2 M, 0.4 mL, 17.04 eq). The mixture was stirred at 25° C. for 1 hr. The reaction mixture was concentrated under reduced pressure to give a residue. The residue was purified by prep-HPLC (column: Phenomenex luna C18 150*25 mm*10 um; mobile phase: [water (HCl)-ACN]; gradient: 24%-54% B over 14 min) and the eluent was lyophilized to give I-478 (16.61 mg, 22.09 μmol, 47.05% yield, 100% purity) as a white solid. LCMS (Method D): Retention time: 0.426 min, [M+H]⁺=752.3. ¹H NMR (400 MHz, METHANOL-d4) δ=8.36 (d, J=7.6 Hz, 1H), 7.97-7.91 (m, 1H), 7.91-7.79 (m, 2H), 7.53-7.44 (m, 3H), 7.44-7.26 (m, 5H), 7.14 (t, J=9.2 Hz, 1H), 4.37 (s, 2H), 4.23-4.11 (m, 2H), 3.86 (d, J=2.8 Hz, 2H), 3.82-3.72 (m, 4H), 3.69 (s, 3H), 3.51 (s, 2H), 3.41-3.32 (m, 4H), 3.30 (s, 2H), 2.80-2.64 (m, 2H), 1.30-1.23 (m, 3H). ¹⁹F NMR (377 MHz, METHANOL-d₄), δ=−111.637, −120.658.

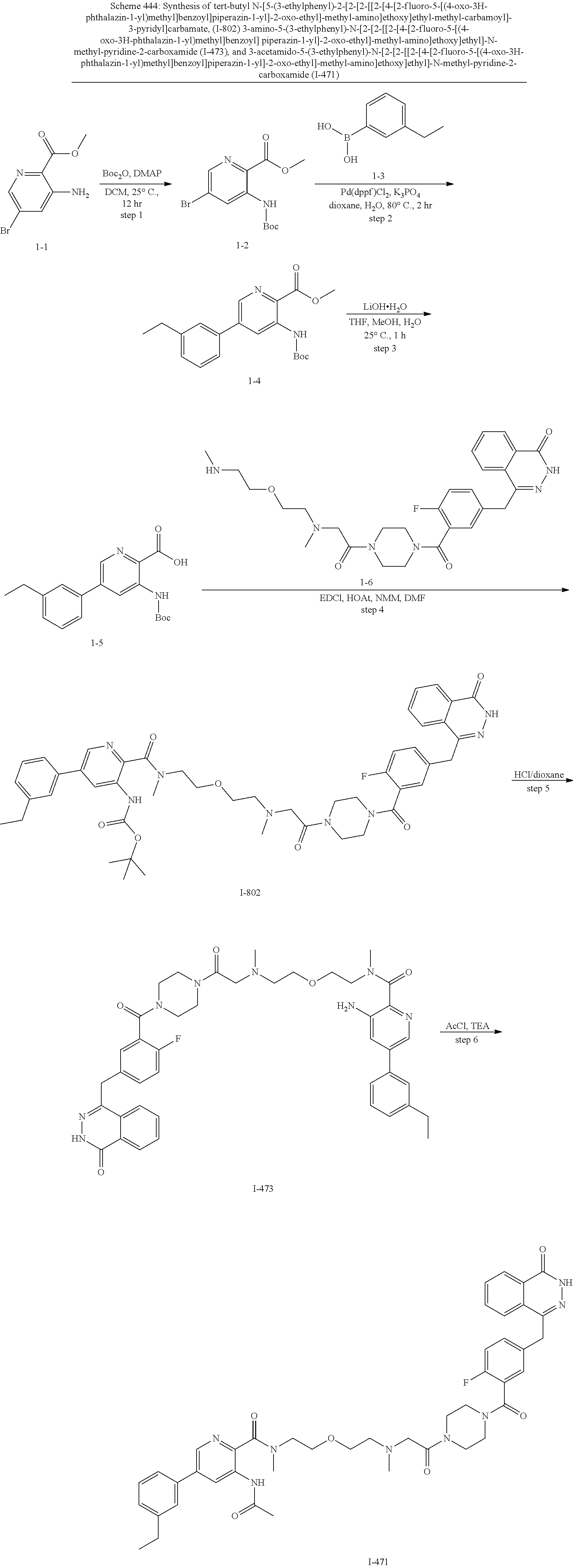

### Step 1: Synthesis of Intermediate 1-2

To a solution of Intermediate 1-1 (2 g, 8.66 mmol, 1 eq) in DCM (20 mL) was added (Boc)₂O (2.27 g, 10.39 mmol, 2.39 mL, 1.2 eq) and DMAP (105.75 mg, 865.63 μmol, 0.1 eq). The mixture was stirred at 25° C. for 12 h. The residue was poured into water (40 mL). The aqueous phase was extracted with DCM (20 mL×3). The combined organic phase was washed with brine (20 mL×3), dried with anhydrous sodium sulfate, filtered, and concentrated in vacuum to give a residue. The residue was purified by flash silica gel chromatography (ISCO; 40 g SepaFlash Silica Flash Column, Eluent of 0˜20% Ethyl acetate/Petroleum ether gradient @100 mL/min) and concentrated under vacuum. Intermediate 1-2 (2.4 g, 7.25 mmol, 83.72% yield) was obtained as a brown solid. ¹H NMR (400 MHz, DMSO-d₆) δ=9.99 (s, 1H), 8.69 (d, J=2.0 Hz, 1H), 8.44 (d, J=2.1 Hz, 1H), 3.86 (s, 3H), 1.48 (s, 9H).

### Step 2: Synthesis of Intermediate 1-4

A mixture of Intermediate 1-2 (2.2 g, 6.64 mmol, 1 eq), Intermediate 1-3 (1.49 g, 9.96 mmol, 1.5 eq), Pd(dppf)Cl₂ (486.09 mg, 664.33 μmol, 0.1 eq) and K₃PO₄ (4.23 g, 19.93 mmol, 3 eq) in dioxane (20 mL) and H₂O (4 mL) was degassed and purged with N₂ 3 times, and then the mixture was stirred at 80° C. for 2 h under N₂ atmosphere. The mixture was poured into water (40 mL). The aqueous phase was extracted with EA (30 mL×3). The combined organic phase was washed with brine (30 mL×3), dried with anhydrous sodium sulfate, filtered, and concentrated in vacuum to give a residue. The residue was purified by flash silica gel chromatography (ISCO; 40 g SepaFlash Silica Flash Column, Eluent of 0˜20% Ethyl acetate/Petroleum ether gradient @100 mL/min) and concentrated under vacuum. Intermediate 1-4 (2.5 g, crude) was obtained as brown oil. LCMS (Method G): Rt=0.826 min, [M+H]⁺=357.2. ¹H NMR (400 MHz, DMSO-d₆) δ=10.02 (s, 1H), 8.69 (d, J=1.8 Hz, 1H), 8.62 (d, J=2.0 Hz, 1H), 7.58-7.50 (m, 2H), 7.49-7.41 (m, 1H), 7.33 (br d, J=7.5 Hz, 1H), 3.88 (s, 3H), 2.72-2.64 (m, 2H), 1.49 (s, 9H), 1.23 (t, J=7.6 Hz, 3H).

### Step 3: Synthesis of Intermediate 1-5

To a solution of Intermediate 1-4 (1.8 g, 5.05 mmol, 1 eq) in THF (8 mL), H₂O (8 mL) and MeOH (4 mL) was added LiOH·H₂O (1.06 g, 25.25 mmol, 5 eq). The mixture was stirred at 25° C. for 1 h. The mixture was concentrated under vacuum to remove THF and MeOH. The aqueous phase was adjusted to pH=7 by 1N HCl and filtered. The filter cake was dried under vacuum to give a crude product. The crude was used for the next step without purification. Intermediate 1-5 (1.3 g, crude) was obtained as a light yellow solid. ¹H NMR (400 MHz, DMSO-d₆) δ=12.96 (s, 1H), 8.88 (br s, 1H), 8.36 (br s, 1H), 7.52-7.38 (m, 3H), 7.30 (br d, J=6.9 Hz, 1H), 2.68 (q, J=7.3 Hz, 2H), 1.50 (s, 9H), 1.21 (br t, J=7.5 Hz, 3H).

### Step 4: Synthesis of I-802

To a solution of Intermediate 1-5 (0.04 g, 116.83 μmol, 1 eq) in DMF (1 mL) was added HOAt (15.90 mg, 116.83 μmol, 16.34 μL, 1 eq), EDCI (44.79 mg, 233.65 μmol, 2 eq), NMM (59.08 mg, 584.13 μmol, 64.22 μL, 5 eq) and Intermediate 1-6 (67.18 mg, 116.83 μmol, 1 eq, HCl). The mixture was stirred at 25° C. for 2 h. The mixture was filtered. The filtrate was purified by Prep-HPLC (column: Phenomenex luna C18 150*25 mm*10 um; mobile phase: [water (FA)-ACN]; gradient: 25%-55% B over 9 min). I-802 (33.92 mg, 39.31 μmol, 33.64% yield, 100% purity) was obtained as a yellow solid. LCMS (Method E): Rt=0.531 min, [M+H]⁺=863.5. ¹H NMR (400 MHz, METHANOL-d₄) δ=8.61-8.43 (m, 2H), 8.37-8.32 (m, 1H), 7.94-7.81 (m, 3H), 7.50-7.28 (m, 7H), 7.19-7.10 (m, 1H), 4.37-4.30 (m, 2H), 3.79-3.62 (m, 8H), 3.54-3.39 (m, 5H), 3.23-3.05 (m, 5H), 2.89-2.80 (m, 1H), 2.75-2.58 (m, 4H), 2.48 (br d, J=17.6 Hz, 1H), 2.31 (br s, 1H), 1.54-1.50 (m, 9H), 1.30-1.22 (m, 4H).

### Step 5: Synthesis of I-473

To a solution of I-802 (36 mg, 41.72 μmol, 1 eq) in DCM (0.5 mL) was added HCl/dioxane (2 M, 1 mL, 47.94 eq). The mixture was stirred at 25° C. for 1 h. The reaction mixture was quenched by sat. NaHCO₃ (2 mL), and extracted with DCM (0.5 mL*3). The combined organic layers were washed with brine (0.5 mL*3), dried over anhydrous Na₂SO₄, filtered and concentrated under reduced pressure to give a residue. The residue was purified by reversed-phase HPLC (0.1% NH₃·H₂O). I-473 (24.88 mg, 30.87 μmol, 73.99% yield, 94.641% purity) was obtained as a white solid. LCMS (Method G): Rt=0.587 min, [M+H]⁺=763.5. ¹H NMR (400 MHz, METHANOL-d₄) δ=8.35 (br d, J=7.4 Hz, 1H), 8.15-7.97 (m, 1H), 7.95-7.77 (m, 3H), 7.51-7.31 (m, 6H), 7.27-7.07 (m, 2H), 4.35 (br d, J=12.0 Hz, 2H), 3.85-3.38 (m, 14H), 3.27-2.96 (m, 5H), 2.79-2.60 (m, 4H), 2.45-2.25 (m, 3H), 1.28-1.22 (m, 3H).

### Step 6: Synthesis of I-471

To a solution of I-473 (40 mg, 52.43 μmol, 1 eq) in DCM (1 mL) was added TEA (26.53 mg, 262.17 μmol, 36.49 μL, 5 eq) and acetyl chloride (4.12 mg, 52.43 μmol, 3.73 μL, 1 eq). The mixture was stirred at 25° C. for 1 h. The reaction mixture was quenched by H₂O (2 mL), and extracted with DCM (1 mL*3). The combined organic layers were washed with brine (13 mL*3), dried over anhydrous Na₂SO₄, filtered and concentrated under reduced pressure to give a residue. The residue was purified by Prep-HPLC (column: Phenomenex luna C18 150*25 mm*10 um; mobile phase: [water (HCl)-MeOH]; gradient: 15%-45% B over 9 min) and reversed-phase HPLC (0.1% NH₃·H₂O). I-471 (7.51 mg, 8.58 μmol, 16.36% yield, 91.936% purity) was obtained as a yellow solid. LCMS (Method G): Rt=0.587 min, [M+H]⁺=805.5. ¹H NMR (400 MHz, METHANOL-d₄) δ=8.63-8.50 (m, 1H), 8.37-8.26 (m, 1H), 8.07-7.69 (m, 3H), 7.51-7.03 (m, 8H), 4.41-4.29 (m, 2H), 3.83-3.39 (m, 14H), 3.27-3.01 (m, 5H), 2.76-2.74 (m, 1H), 2.78-2.66 (m, 2H), 2.63-2.53 (m, 1H), 2.42-2.23 (m, 3H), 2.22-2.13 (m, 3H), 1.30-1.23 (m, 3H).

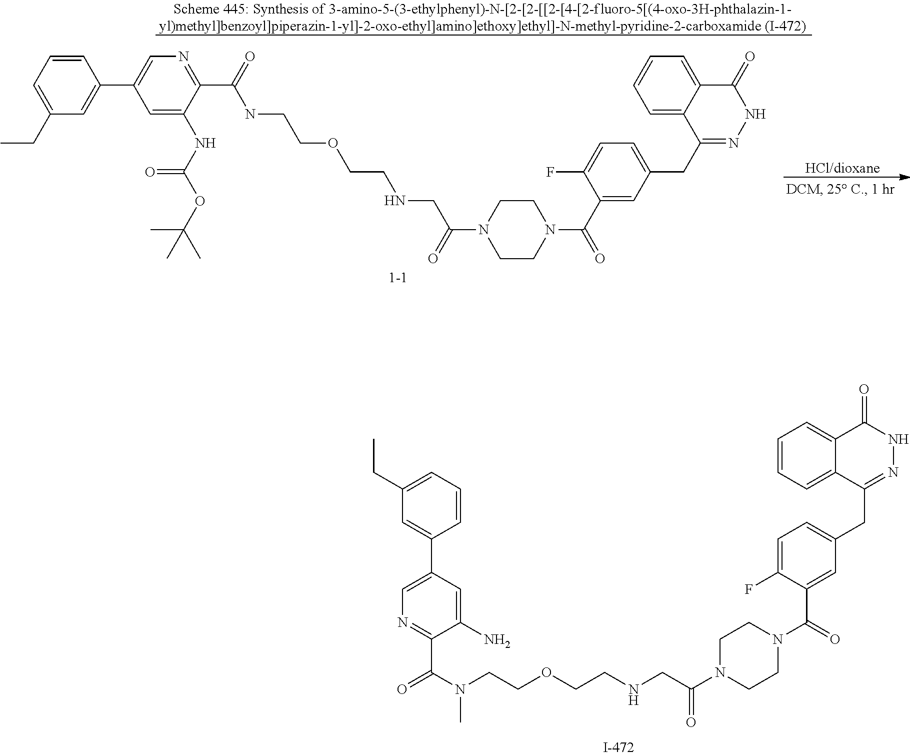

To a solution of Intermediate 1-1 (100 mg, 117.79 μmol, 1 eq) in DCM (1 mL) was added HCl/dioxane (2 M, 2 mL, 33.96 eq). The mixture was stirred at 25° C. for 1 hr. The reaction mixture was concentrated directly under vacuum. The product was purified by prep-HPLC (column: Phenomenex luna C18 150*25 mm*10 um; mobile phase: [water (HCl)-ACN]; gradient: 18%-48% B over 10 min) and the eluent was concentrated and then lyophilized. I-472 (18 mg, 22.69 μmol, 19.26% yield, 99% purity, HCl salt) was obtained as a white solid. LCMS (Method E): Rt=0.443 min, [M+H]⁺=749.5. ¹H NMR (400 MHz, METHANOL-d₄) δ=8.39-8.33 (m, 2H), 7.99-7.93 (m, 2H), 7.90-7.80 (m, 2H), 7.60-7.47 (m, 3H), 7.47-7.41 (m, 1H), 7.40-7.31 (m, 2H), 7.20-7.11 (m, 1H), 4.38 (s, 2H), 4.20 (m, 2H), 3.90-3.69 (m, 8H), 3.68-3.62 (m, 1H), 3.57-3.46 (m, 2H), 3.39 (s, 3H), 3.26 (s, 2H), 3.18 (d, J=3.6 Hz, 3H), 2.73 (m, 2H), 1.31-1.22 (m, 3H). ¹⁹F NMR (377 MHz, METHANOL-d₄) δ=−120.666.

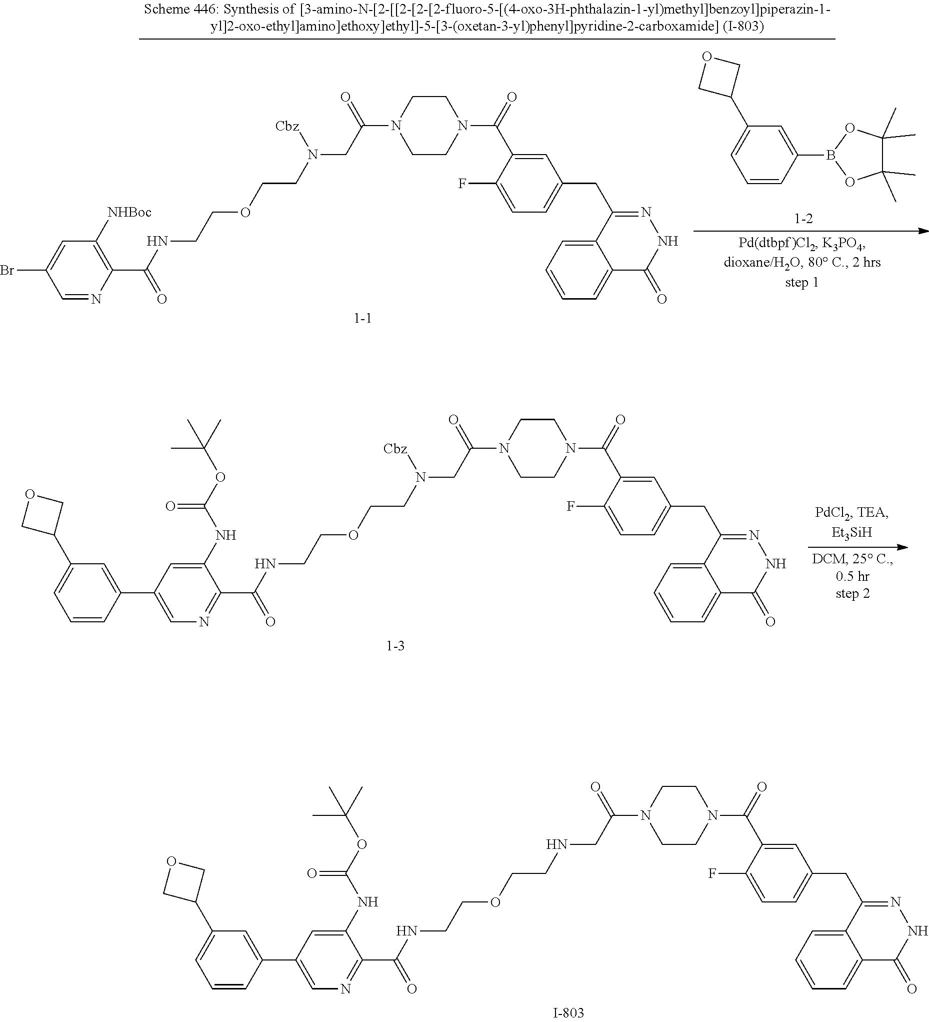 [SMILES: CC(C)(C)OC(=O)Nc1cc(Br)cnc1C(=O)NCCOCCN(CC(=O)N1CCN(C(=O)c2cc(Cc3n[nH]c(=O)c4ccccc34)ccc2F)CC1)C(=O)OCc1ccccc1 ; CC1(C)OB(c2cccc(C3COC3)c2)OC1(C)C ; CC(C)(C)OC(=O)Nc1cc(-c2cccc(C3COC3)c2)cnc1C(=O)NCCOCCN(CC(=O)N1CCN(C(=O)c2cc(Cc3n[nH]c(=O)c4ccccc34)ccc2F)CC1)C(=O)OCc1ccccc1 ; CC(C)(C)OC(=O)Nc1cc(-c2cccc(C3COC3)c2)cnc1C(=O)NCCOCCNCC(=O)N1CCN(C(=O)c2cc(Cc3n[nH]c(=O)c4ccccc34)ccc2F)CC1]

### Step 1: Synthesis of Intermediate 1-3.

A mixture of Intermediate 1-1 (280 mg, 296.67 μmol, 1 eq), Intermediate 1-2 (77.17 mg, 296.67 μmol, 1 eq), Pd(dtbpf)Cl₂ (19.34 mg, 29.67 μmol, 0.1 eq), and K₃PO₄ (188.92 mg, 890.01 μmol, 3 eq) in dioxane (2.5 mL) and H₂O (0.5 mL) was degassed and purged with N₂ 3 times, and then the mixture was stirred at 80° C. for 2 hr under N₂ atmosphere. The reaction mixture was diluted with H₂O (2 mL) and extracted with DCM (0.8 mL*3), dried over Na₂SO₄, filtered and concentrated under reduced pressure to give a crude product. The crude product was purified by reversed-phase chromatography (neutral condition) and lyophilized to give Intermediate 1-3 (150 mg, 150.44 μmol, 50.71% yield, 100% purity) as a white solid. LCMS (Method D): Retention time: 0.575 min, [M+H]⁺=997.4.

### Step 2: Synthesis of I-803

To a solution of Intermediate 1-3 (50 mg, 50.15 μmol, 1 eq) in DCM (0.5 mL) was added PdCl₂ (4.45 mg, 25.07 μmol, 0.5 eq) and TEA (15.22 mg, 150.44 μmol, 20.94 μL, 3 eq). The reaction mixture was degassed with N₂. Then Et₃SiH (69.97 mg, 601.76 μmol, 96.11 μL, 12 eq) was added in. The mixture was stirred at 25° C. for 1 hr. The pH of the reaction mixture was to slightly alkaline by NaHCO₃, diluted with H₂O (0.5 mL), extracted with DCM (0.2 mL*3), dried over Na₂SO₄, filtered and concentrated under reduced pressure to give a crude product. The crude product was purified by prep-HPLC (column: Phenomenex luna C18 150*25 mm*10 um; mobile phase: [water(FA)-ACN]; gradient: 23%-533% B over 10 min) and lyophilized to give the product. I-803 (12.37 mg, 13.61 μmol, 27.14% yield, 100% purity, FA salt) was obtained as a white solid. LCMS (Method D): Retention time: 0.388 min, [M+H]⁺=863.3. ¹H NMR (400 MHz, METHANOL-d4) δ=9.02 (s, 1H), 8.53 (s, 1H), 8.52-8.45 (m, 1H), 8.36 (d, J=6.8 Hz, 1H), 7.96-7.90 (m, 1H), 7.89-7.78 (m, 2H), 7.70 (s, 1H), 7.64-7.42 (m, 4H), 7.37-7.28 (m, 1H), 7.19-7.07 (m, 1H), 5.19-5.07 (m, 2H), 4.82-4.77 (m, 2H), 4.40 (s, 3H), 3.75-3.58 (m, 11H), 3.50-3.45 (m, 2H), 3.24 (d, J=3.2 Hz, 3H), 2.94 (d, J=3.6 Hz, 2H), 1.54 (s, 9H). ¹⁹F NMR (376 MHz, METHANOL-d₄) δ=−120.642.

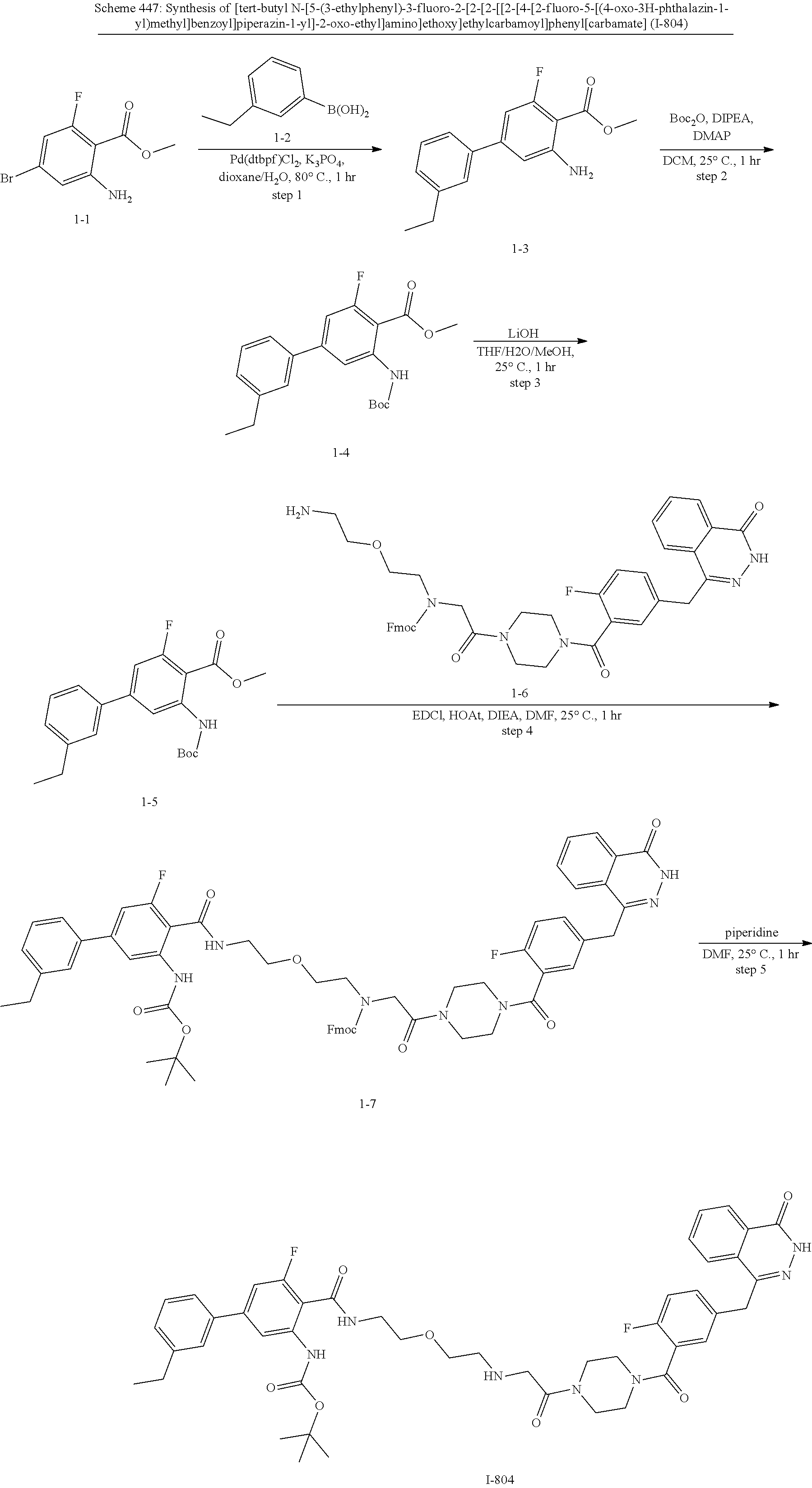

### Step 1: Synthesis of Intermediate 1-3

To a solution of Intermediate 1-1 (500 mg, 2.02 mmol, 1 eq) and Intermediate 1-2 (362.79 mg, 2.42 mmol, 1.2 eq) in dioxane (5 mL) and H₂O (1 mL) was added Pd(dtbpf)Cl₂ (131.37 mg, 201.57 μmol, 0.1 eq) and K₃PO₄ (855.74 mg, 4.03 mmol, 2 eq). The mixture was stirred at 80° C. for 1 h. The reaction mixture was diluted with H₂O (10 mL) at 25° C. and extracted with EA (10 ml*3), and the combined organic phase was dried with anhydrous sodium sulfate, filtered and the filtrate was concentrated to give crude product. The crude product was purified by column chromatography (SiO₂, PE/EA=1/0 to 0/1) and the eluent was concentrated under reduced pressure to give Intermediate 1-3 (520 mg, 1.86 mmol, 92.50% yield, 98% purity) as a yellow oil. LCMS (Method D): Retention time: 0.576 min, (M+H)=274.1.

### Step 2: Synthesis of Intermediate 1-4

To a solution of Intermediate 1-3 (500 mg, 1.83 mmol, 1 eq) in DCM (5 mL) was added Boc₂O (598.92 mg, 2.74 mmol, 630.44 μL, 1.5 eq), DMAP (22.35 mg, 182.95 μmol, 0.1 eq) and DIEA (472.89 mg, 3.66 mmol, 637.32 μL, 2 eq). The mixture was stirred at 25° C. for 1 h. The reaction mixture was concentrated under reduced pressure to give a residue. The crude product was purified by column chromatography (SiO₂, PE/EA=1/0 to 0/1) and the eluent was concentrated under reduced pressure to give Intermediate 1-4 (600 mg, 1.03 mmol, 56.10% yield, 81% purity) as a yellow oil. LCMS (Method D): Retention time: 0.508 min, (M−2Boc)=274.1. ¹H NMR (400 MHz, METHANOL-d4) δ=7.55-7.46 (m, 3H), 7.43-7.37 (m, 2H), 7.29 (d, J=7.6 Hz, 1H), 3.89 (s, 3H), 2.76-2.70 (m, 2H), 1.39 (s, 18H), 1.30-1.26 (m, 3H). ¹⁹F NMR (400 MHz, METHANOL-d4) δ=−113.198.

### Step 3: Synthesis of Intermediate 1-5

To a solution of Intermediate 1-4 (550 mg, 1.16 mmol, 1 eq) in MeOH (2 mL), THF (2 mL) and H₂O (2 mL) was added LiOH·H₂O (146.21 mg, 3.48 mmol, 3 eq). The mixture was stirred at 25° C. for 1 h. The mixture was diluted with 2 ml of H₂O and adjusted to pH to 5-6 with 1M HCl, and then extracted with EA (3 ml*3). The combined organic phase was dried with anhydrous sodium sulfate, filtered and the filtrate was concentrated to give Intermediate 1-5 (420 mg, crude) as a white solid. LCMS (Method D): Retention time: 0.485 min, (M−2Boc)=260.1.

### Step 4: Synthesis of Intermediate 1-7

To a solution of Intermediate 1-5 (168.19 mg, 366.02 μmol, 9.39e−1 eq) in DMF (3 mL) was added EDCI (224.28 mg, 1.17 mmol, 3 eq), HOAt (53.08 mg, 389.99 μmol, 54.55 μL, 1 eq), NMM (197.23 mg, 1.95 mmol, 214.38 μL, 5 eq) and Intermediate 1-6 (300 mg, 389.99 μmol, 1 eq, HCl salt). The mixture was stirred at 25° C. for 1 h. The reaction mixture was diluted with H₂O (10 mL) at 25° C. and extracted with EA (10 ml*3). The combined organic phase was dried with anhydrous sodium sulfate, filtered and the filtrate was concentrated to give the crude product. The crude product was purified by column chromatography (SiO₂, PE/EA=1/0 to 0/1) and the eluent was concentrated under reduced pressure to give Intermediate 1-7 (400 mg, 350.04 μmol, 89.76% yield, 94% purity) as a yellow solid. LCMS (Method D): Retention time: 0.537 min, (M+H)=1074.4. ¹H NMR (400 MHz, METHANOL-d4) δ=8.34 (t, J=6.0 Hz, 1H), 8.29-8.11 (m, 1H), 7.91 (d, J=7.6 Hz, 1H), 7.86-7.76 (m, 3H), 7.74-7.59 (m, 2H), 7.49 (d, J=7.6 Hz, 1H), 7.43-7.22 (m, 10H), 7.16-6.96 (m, 2H), 4.55-4.48 (m, 2H), 4.40-4.04 (m, 5H), 3.95-3.85 (m, 1H), 3.69 (d, J=4.8 Hz, 1H), 3.57 (s, 4H), 3.29-3.00 (m, 7H), 2.72-2.65 (m, 2H), 1.51-1.38 (m, 9H), 1.26-1.22 (m, 3H).

### Step 5: Synthesis of I-804

To a solution of Intermediate 1-7 (50 mg, 46.55 μmol, 1 eq) in DMF (0.5 mL) was added piperidine (11.89 mg, 139.64 μmol, 13.79 μL, 3 eq). The mixture was stirred at 25° C. for 1 h. The reaction mixture was purified by prep-HPLC (column: CD04-Welch Utimate C18 150*25*7 um; mobile phase: [water(FA)-ACN]; gradient: 27%-577% B over 8 min) and lyophilized to give I-804 (17.35 mg, 19.09 μmol, 41.01% yield, 98.792% purity, FA salt) as a white solid. LCMS (Method D): Retention time: 0.503 min, (M+H)=852.4. ¹H NMR (400 MHz, METHANOL-d4) δ=8.50 (s, 1H), 8.36 (d, J=7.6 Hz, 1H), 8.21 (d, J=4.4 Hz, 1H), 7.96-7.75 (m, 3H), 7.50-7.20 (m, 6H), 7.13-7.11 (m, 2H), 4.35 (d, J=3.6 Hz, 2H), 3.90-3.76 (m, 2H), 3.76-3.54 (m, 9H), 3.52-3.42 (m, 2H), 3.24 (d, J=11.6 Hz, 3H), 3.06 (s, 2H), 2.79-2.59 (m, 2H), 1.53 (s, 9H), 1.32-1.19 (m, 3H). ¹⁹F NMR (400 MHz, METHANOL-d4) δ=−113.685, −120.605.

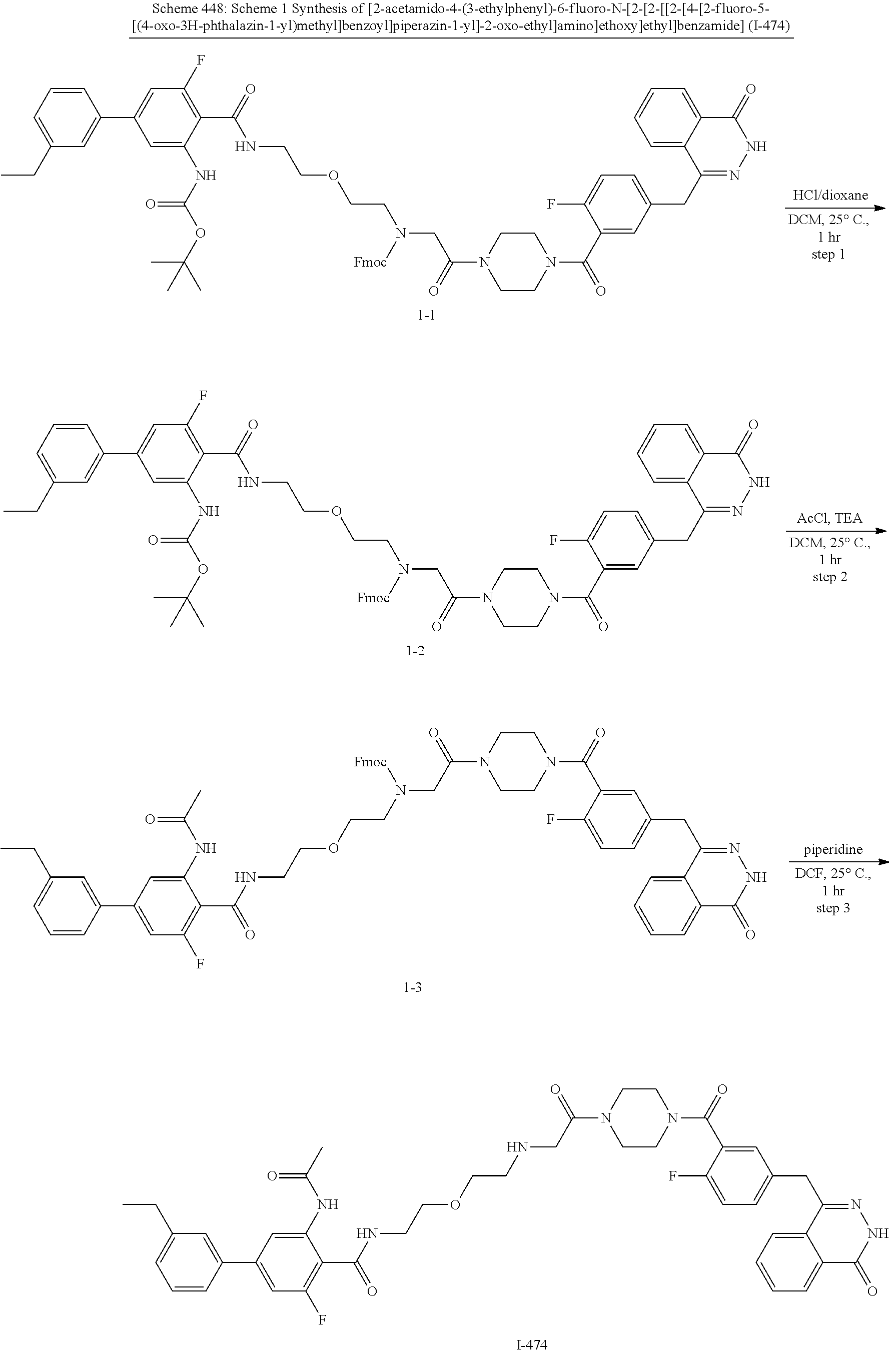

### Step 1: Synthesis of Intermediate 1-2

A solution of Intermediate 1-1 (150 mg, 139.64 μmol, 1 eq) in HCl/dioxane (2 M, 1.50 mL, 21.48 eq) was stirred at 25° C. for 1 h. The reaction mixture was concentrated under reduced pressure to give Intermediate 1-2 (150 mg, crude, HCl salt) as a white solid. LCMS (Method D): Retention time: 0.583 min, [M+H]⁺=974.4.

### Step 2: Synthesis of Intermediate 1-3

To a solution of Intermediate 1-2 (150 mg, 148.44 μmol, 1 eq, HCl salt) in DCM (1.5 mL) was added TEA (45.06 mg, 445.32 μmol, 61.98 μL, 3 eq) and acetyl chloride (23.30 mg, 296.88 μmol, 21.11 L, 2 eq) at 0° C. The mixture was stirred at 25° C. for 1 h. The reaction mixture was diluted with H₂O (2 mL) at 25° C. and extracted with DCM (2 ml*3). The combined organic phase was dried with anhydrous sodium sulfate, filtered and the filtrate was concentrated to give crude product. The crude product was purified by reversed phase chromatography (0.1% FA condition) and the eluent was lyophilized to give Intermediate 1-3 (50 mg, 46.75 μmol, 31.49% yield, 95% purity) as a yellow solid. LCMS (Method D): Retention time: 0.580 min, [M+H]⁺=1016.3.

### Step 3: Synthesis of I-474

To a solution of Intermediate 1-3 (50 mg, 49.21 μmol, 1 eq) in DMF (0.5 mL) was added piperidine (12.57 mg, 147.62 μmol, 14.58 μL, 3 eq). The mixture was stirred at 25° C. for 0.5 h. The reaction mixture was purified by reversed phase chromatography (0.10% FA condition) and the eluent was lyophilized to give I-474 (12.97 mg, 15.44 μmol, 31.38% yield, 100% purity, FA salt) as a white solid. LCMS (Method D): Retention time: 0.383 min, (M+H)=794.4. ¹H NMR (400 MHz, METHANOL-d4) δ=8.50 (s, 1H), 8.36 (d, J=7.6 Hz, 1H), 8.03 (s, 1H), 7.95-7.90 (m, 1H), 7.89-7.79 (m, 2H), 7.50-7.39 (m, 3H), 7.37-7.20 (m, 4H), 7.12 (t, J=9.2 Hz, 1H), 4.35 (s, 2H), 3.94-3.78 (m, 2H), 3.74 (s, 2H), 3.69 (s, 3H), 3.63 (s, 3H), 3.51-3.42 (m, 2H), 3.34 (s, 2H), 3.24 (d, J=9.2 Hz, 2H), 3.09 (s, 2H), 2.76-2.63 (m, 2H), 2.17 (d, J=5.8 Hz, 3H), 1.30-1.21 (m, 3H). ¹⁹F NMR (400 MHz, METHANOL-d4) δ=−76.933, −114.406, −120.635.

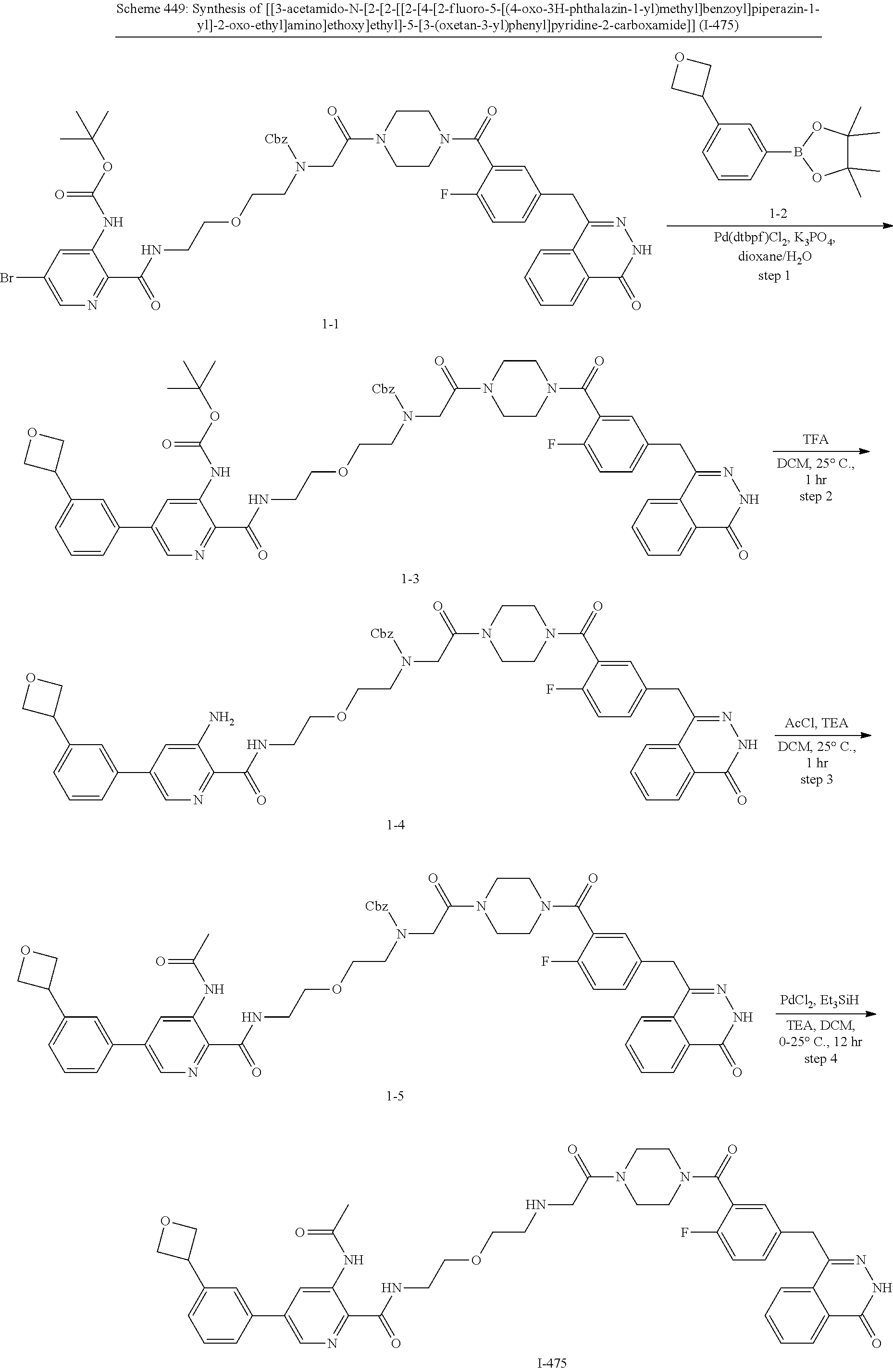

### Step 1: Synthesis of Intermediate 1-3

To a solution of intermediate 1-1 (200 mg, 211.91 μmol, 1 eq) and intermediate 1-2 (66.15 mg, 254.29 μmol, 1.2 eq) in dioxane (2 mL) and H₂O (0.4 mL) was added Pd(dtbpf)Cl₂ (13.81 mg, 21.19 μmol, 0.1 eq) and K₃PO₄ (134.94 mg, 635.72 μmol, 3 eq). The mixture was stirred at 80° C. for 1 h. The reaction mixture was diluted with H₂O (5 mL), and the mixture was extracted with EA (5 ml*3). The combined organic phase was dried with anhydrous sodium sulfate, filtered, and the filtrate was concentrated to give intermediate 1-3 (120 mg, 117.95 μmol, 55.66% yield, 98% purity) as a yellow oil. LCMS (Method D): Retention time: 0.489 min, [M+H]⁺=997.3.

### Step 2: Synthesis of Intermediate 1-4

To a solution of intermediate 1-3 (100 mg, 100.29 μmol, 1 eq) in DCM (0.9 mL) was added TFA (11.44 mg, 100.29 μmol, 0.3 mL, 1 eq). The mixture was stirred at 25° C. for 1 h. The reaction mixture was concentrated under reduced pressure to give intermediate 1-4 (100 mg, crude, TFA salt) as a yellow oil. LCMS (Method D): Retention time: 0.427 min, [M+H]⁺=897.3.

### Step 3: Synthesis of Intermediate 1-5

To a solution of intermediate 1-4 (100 mg, 98.91 μmol, 1 eq, TFA salt) in DCM (1 mL) was added TEA (30.03 mg, 296.74 μmol, 41.30 μL, 3 eq) and acetyl chloride (7.76 mg, 98.91 μmol, 7.03 μL, 1 eq) at 0° C. The mixture was stirred at 25° C. for 1 h. The reaction mixture was diluted with H₂O (3 mL) and extracted with EA (3 ml*3). The combined organic phase was dried with anhydrous sodium sulfate, filtered and concentrated to give crude product. The crude product was purified by reversed phase chromatography (0.1% FA condition) and the eluent was lyophilized to give intermediate 1-5 (30 mg, 31.63 μmol, 31.98% yield, 99% purity) as a white solid. LCMS (Method D): Retention time: 0.435 min, [M+H]⁺=939.3.

### Step 4: Synthesis of I-475

To a solution of intermediate 1-5 (100 mg, 100.29 μmol, 1 eq) in DCM (0.9 mL) was added TFA (11.44 mg, 100.29 μmol, 0.3 mL, 1 eq). The mixture was stirred at 25° C. for 1 h. The reaction mixture was concentrated under reduced pressure to give I-475 (6.43 mg, 7.33 μmol, 22.94% yield, 96.996% purity, FA salt) as a yellow solid. LCMS (Method D): Retention time: 0.376 min, [M+H]⁺=805.4. ¹H NMR (400 MHz, METHANOL-d4) δ=9.27 (s, 1H), 8.58 (d, J=11.6 Hz, 1H), 8.51-8.42 (m, 1H), 8.36 (d, J=7.2 Hz, 1H), 7.97-7.89 (m, 1H), 7.89-7.79 (m, 2H), 7.70 (s, 1H), 7.61-7.46 (m, 4H), 7.31 (s, 1H), 7.17-7.11 (m, 1H), 5.16-5.08 (m, 2H), 4.81-4.75 (m, 2H), 4.41-4.31 (m, 3H), 3.98-3.85 (m, 2H), 3.82-3.59 (m, 10H), 3.46 (d, J=4.8 Hz, 2H), 3.25 (d, J=3.6 Hz, 2H), 3.18-3.14 (m, 2H), 2.25 (d, J=2.0 Hz, 3H). ¹⁹F NMR (377 MHz, METHANOL-d4) δ=−120.635.

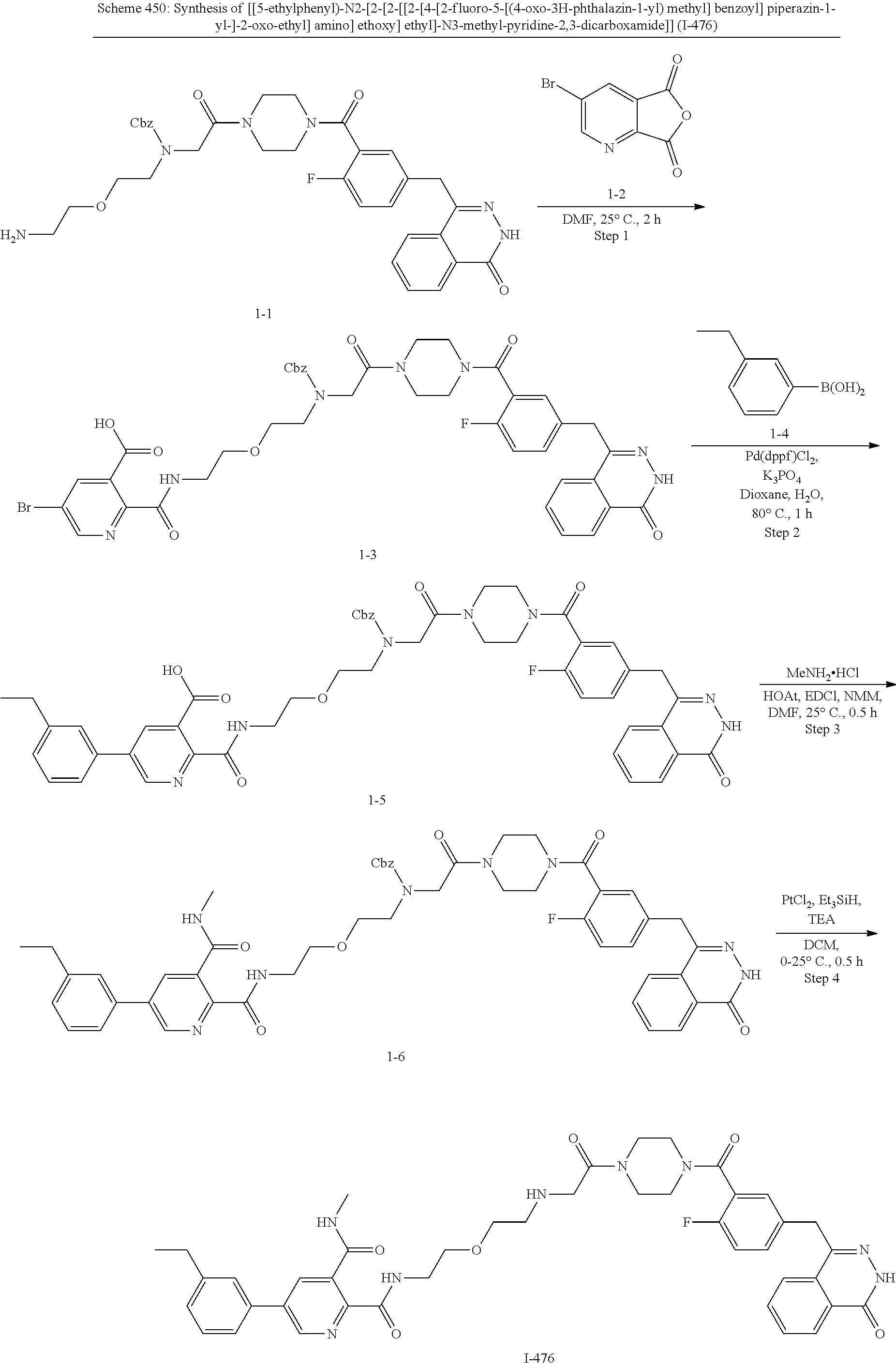

### Step 1: Synthesis of Intermediate 1-3

To a solution of intermediate 1-1 (400 mg, 587.24 μmol, 1 eq, HCl salt) in DMF (5 mL) was added intermediate 1-2 (401.67 mg, 1.76 mmol, 3 eq). The mixture was stirred at 25° C. for 2 hr. The reaction mixture was purified by reversed phase chromatography (neutral condition). The eluent was concentrated to remove MeCN and then lyophilized to afford Intermediate 1-3 (340 mg, crude) as a yellow solid. LCMS (Method D): Rt=0.384 min, [M+H]⁺=872.1.

### Step 2: Synthesis of Intermediate 1-5

A mixture of intermediate 1-3 (340 mg, 389.60 μmol, 1 eq), intermediate 1-4 (75.96 mg, 506.48 μmol, 1.3 eq), Pd(dtbpf)Cl₂ (25.39 mg, 38.96 μmol, 0.1 eq) and K₃PO₄ (248.10 mg, 1.17 mmol, 3 eq) in dioxane (4 mL) and H₂O (0.8 mL) was degassed and purged with N₂ 3 times, and then the mixture was stirred at 80° C. for 3 hr under N₂ atmosphere. The reaction mixture was concentrated under reduced pressure to give a residue. The residue was purified by reversed phase chromatography (0.1% FA). The eluent was concentrated to remove MeCN and then lyophilized to afford Intermediate 1-5 (290 mg, crude) as a brown solid. LCMS (Method D): Rt=0.418 min, [M+H]⁺=892.2.

### Step 3: Synthesis of Intermediate 1-6

To a solution of intermediate 1-5 (240 mg, 267.28 μmol, 1 eq) and methanamine hydrochloride (21.66 mg, 320.73 μmol, 1.2 eq) in DMF (2.4 mL) was added EDCI (153.71 mg, 801.83 μmol, 3 eq), HOAt (36.38 mg, 267.28 μmol, 37.39 μL, 1 eq) and NMM (135.17 mg, 1.34 mmol, 146.93 μL, 5 eq). The mixture was stirred at 25° C. for 0.5 hr. The reaction mixture was poured into H₂O (5 mL), and extracted with EA (5 mL*3). The combined organic layers were dried over Na₂SO₄, filtered and concentrated under reduced pressure to give a residue. The residue was purified by flash silica gel chromatography (ISCO; 12 g SepaFlashSilica Flash Column, Eluent of 0˜100% DCM/MeOH@36 mL/min, DCM/MeOH=10:1, Rf=0.5) and the eluent was concentrated under reduced pressure to give a product. Intermediate 1-6 (180 mg, crude) was obtained as a brown solid. LCMS (Method D): Rt=0.425 min, [M+H]⁺=911.3.

### Step 4: Synthesis of I-476

To a solution of intermediate 1-6 (70 mg, 76.84 μmol, 1 eq) in DCM (0.7 mL) was added PdCl₂ (6.81 mg, 38.42 μmol, 0.5 eq), TEA (23.33 mg, 230.52 μmol, 32.09 μL, 3 eq) and Et₃SiH (107.22 mg, 922.08 μmol, 147.28 μL, 12 eq). The mixture was stirred at 25° C. for 0.5 hr. The reaction mixture was concentrated under reduced pressure to give a residue. The residue was purified by reversed-phase chromatography (0.1% FA). The eluent was concentrated to remove MeCN and then lyophilized to afford I-476 (8.66 mg, 10.52 μmol, 13.70% yield, 100% purity, FA salt) as a white solid. LCMS (Method F): Rt=0.546 min, (M+H)=777.4. ¹H NMR (400 MHz, METHANOL-d4) δ=8.94-8.84 (m, 1H), 8.47 (s, 1H), 8.36 (d, J=7.6 Hz, 1H), 8.14-8.01 (m, 1H), 7.98-7.91 (m, 1H), 7.90-7.78 (m, 2H), 7.63-7.46 (m, 3H), 7.46-7.39 (m, 1H), 7.38-7.25 (m, 2H), 7.19-7.04 (m, 1H), 4.44-4.26 (m, 2H), 4.16-3.96 (m, 2H), 3.86-3.61 (m, 9H), 3.50 (d, J=4.8 Hz, 2H), 3.35 (s, 2H), 3.29-3.16 (m, 3H), 3.01-2.84 (m, 3H), 2.80-2.62 (m, 2H), 1.33-1.22 (m, 3H). ¹⁹F NMR (377 MHz, METHANOL-d4) δ=−120.667.

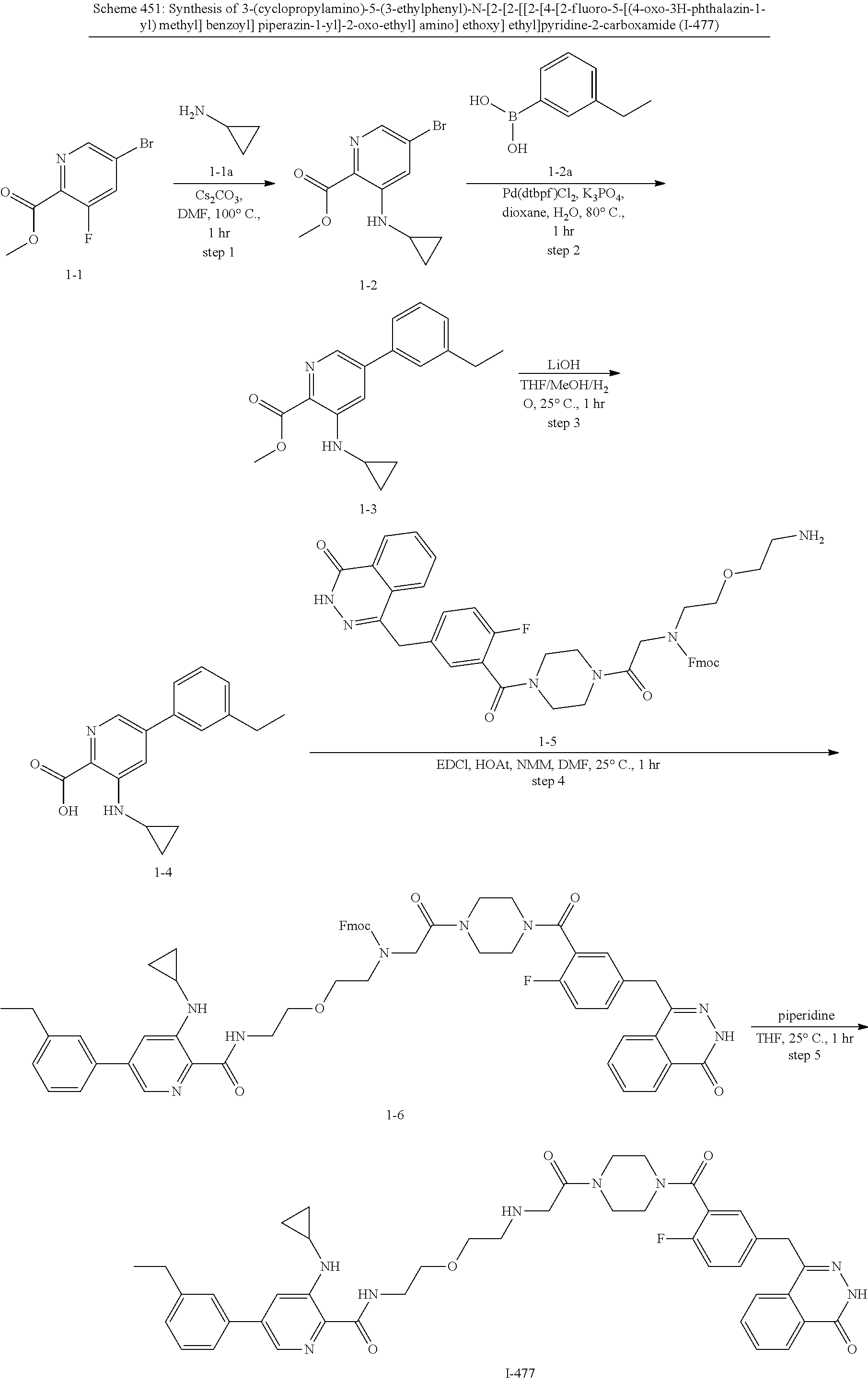

### Step 1: Synthesis of Intermediate 1-2.

To a solution of intermediate 1-1 (150 mg, 640.96 μmol, 1 eq) in DMF (1.5 mL) was added intermediate 1-1a (43.91 mg, 769.16 μmol, 53.29 μL, 1.2 eq) and Cs₂CO₃ (626.52 mg, 1.92 mmol, 3 eq). The mixture was stirred at 100° C. for 1 hr. The reaction mixture was diluted with H₂O (1 mL) and extracted with EA (1 mL*2). The combined organic layers were dried over Na₂SO₄, filtered and concentrated under reduced pressure to give a residue. The residue was purified by flash silica gel chromatography (ISCO; 12 g SepaFlash Silica Flash Column, Eluent of 0˜30% Ethylacetate/Petroleum ethergradient @60 mL/min). The eluent was concentrated under reduced pressure to give intermediate 1-2 (150 mg, 553.28 mol, 86.32% yield, 100% purity) as a white solid. LCMS (Method D): Retention time: 0.393 min, [M+H]⁺=270.9. ¹H NMR (400 MHz, METHANOL-d₄) δ=7.91 (d, J=2.0 Hz, 1H), 7.82 (d, J=2.0 Hz, 1H), 3.90 (s, 3H), 2.58-2.50 (m, 1H), 0.93-0.88 (m, 2H), 0.59-0.54 (m, 2H).

### Step 2: Synthesis of Intermediate 1-3.

To a solution of intermediate 1-2 (130 mg, 479.51 μmol, 1 eq) in H₂O (0.3 mL) and 1,4-dioxane (1.5 mL) was added intermediate 1-2a (86.30 mg, 575.41 μmol, 1.2 eq), K₃PO₄ (305.35 mg, 1.44 mmol, 3 eq) and Pd(dtbpf)Cl₂ (31.25 mg, 47.95 μmol, 0.1 eq). The mixture was stirred at 80° C. for 1 hr under N₂. The reaction mixture was diluted with H₂O (10 mL) and extracted with EA (10 mL*2). The combined organic layers were dried over Na₂SO₄, filtered and concentrated under reduced pressure to give a residue. The residue was purified by flash silica gel chromatography (ISCO; 12 g SepaFlash Silica Flash Column, Eluent of 0˜30% Ethyl acetate/Petroleum ether gradient @40 mL/min). The eluent was concentrated under reduced pressure to give the product. Intermediate 1-3 (140 mg, 472.39 μmol, 98.52% yield) was obtained as a yellow oil. LCMS (Method D): Retention time: 0.446 min, [M+H]⁺=297.1.

### Step 3: Synthesis of Intermediate 1-4.

To a solution of intermediate 1-3 (120 mg, 404.91 μmol, 1 eq) in MeOH (0.5 mL), THF (0.5 mL), and H₂O (0.5 mL) was added LiOH H₂O (50.97 mg, 1.21 mmol, 3 eq). The mixture was stirred at 25° C. for 1 hr. The mixture was diluted with H₂O (5 mL) and HCl (1M) was added to adjust the pH to 4. The mixture was extracted with EA (5 mL*2). The combined organic layers were dried over Na₂SO₄, filtered and concentrated under reduced pressure to give a residue. The residue was purified by reversed-phase chromatography (neutral condition). The eluent was concentrated under reduced pressure to remove ACN and then lyophilized to give intermediate 1-4 (90 mg, 318.77 μmol, 78.73% yield) as a yellow solid. LCMS (Method D): Retention time: 0.394 min, [M+H]⁺=293.0.

### Step 4: Synthesis of Intermediate 1-6.

To a solution of intermediate 1-4 (73.40 mg, 259.99 μmol, 1 eq) in DMF (2 mL) was added intermediate 1-5 (200 mg, 259.99 μmol, 1 eq, HCl salt), EDCI (149.52 mg, 779.97 μmol, 3 eq), HOAt (35.39 mg, 259.99 μmol, 36.37 μL, 1 eq) and NMM (131.49 mg, 1.30 mmol, 142.92 μL, 5 eq). The mixture was stirred at 25° C. for 1 hr. The reaction mixture was diluted with H₂O (10 mL) and extracted with EA (10 mL*2). The combined organic layers were dried over Na₂SO₄, filtered and concentrated under reduced pressure to give a residue. The combined organic layers were washed with brine (2 mL*1), dried over Na₂SO₄, filtered and concentrated under reduced pressure to give a residue. The residue was purified by flash silica gel chromatography (ISCO; 20 g SepaFlash Silica Flash Column, Eluent of 0˜30% Ethylacetate/Petroleum ethergradient @60 mL/min). The eluent was concentrated under reduced pressure to give intermediate 1-6 (80 mg, 74.05 μmol, 28.48% yield, 92.3% purity) as yellow gun. LCMS (Method D): Retention time: 0.568 min, [M+H]⁺=997.6.

### Step 5: Synthesis of I-477

To a solution of intermediate 1-6 (70 mg, 70.20 μmol, 1 eq) in THF (1 mL) was added piperidine (12.07 mg, 141.76 μmol, 14.00 μL, 2.02 eq). The mixture was stirred at 25° C. for 1 hr. The reaction mixture was diluted with H₂O (5 mL) and extracted with EA (5 mL*2). The combined organic layers were dried over Na₂SO₄, filtered and concentrated under reduced pressure to give a residue. The residue was purified by prep-HPLC (column: CD04-Welch Utimate C18 150*25*7 um; mobile phase: [water (FA)-ACN]; gradient: 22%-52% B over 8 min). The eluent was concentrated under reduced pressure to remove ACN and then lyophilized to give I-477 (5.81 mg, 7.15 μmol, 10.18% yield, 95.354% purity) as a white solid. LCMS (Method D): Retention time: 0.381 min, [M+H]⁺=775.5. ¹H NMR (400 MHz, METHANOL-d₄) δ=8.36 (d, J=7.6 Hz, 1H), 8.11-8.03 (m, 1H), 7.95-7.90 (m, 1H), 7.89-7.79 (m, 2H), 7.69-7.63 (m, 1H), 7.50-7.37 (m, 4H), 7.36-7.24 (m, 2H), 7.16-7.08 (m, 1H), 4.39-4.32 (m, 2H), 4.21-4.09 (m, 2H), 3.86-3.79 (m, 2H), 3.78-3.55 (m, 8H), 3.52-3.42 (m, 2H), 3.35-3.32 (m, 2H), 3.30-3.22 (m, 2H), 2.77-2.62 (m, 2H), 2.59-2.49 (m, 1H), 1.30-1.20 (m, 3H), 0.91-0.81 (m, 2H), 0.59-0.49 (m, 2H). ¹⁹FNMR (377 MHz, MeOD-d₆) δ=−120.539, −122.573.

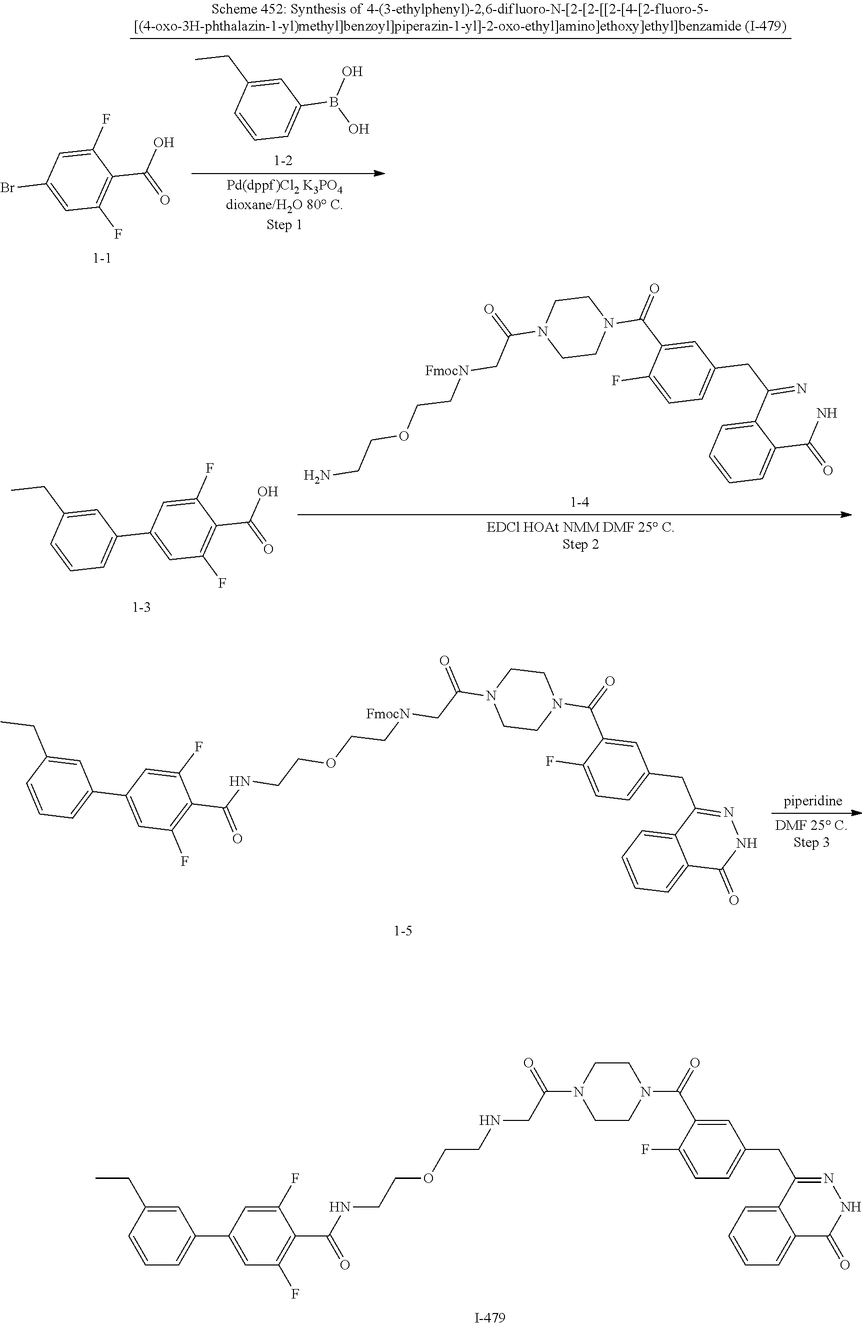

### Step 1: Synthesis of Intermediate 1-3

A mixture of Intermediate 1-1 (100 mg, 421.94 μmol, 1 eq), Intermediate 1-2 (63.28 mg, 421.94 μmol, 1 eq), K₃PO₄ (268.69 mg, 1.27 mmol, 3 eq), Pd(dppf)Cl₂ (61.75 mg, 84.39 μmol, 0.2 eq) in dioxane (0.8 mL) and H₂O (0.2 mL) was degassed and purged with N₂ 3 times, and then the mixture was stirred at 80° C. for 1 hr under N₂ atmosphere. The reaction mixture was quenched by addition of water (5 mL) at 25° C., and then extracted with EA (5 mL*3). Next, the pH of the aqueous phase was adjusted to 5-6, and then extracted with EA (5 mL*3). The organic phase was dried over Na₂SO₄, filtered and concentrated under reduced pressure to give a residue. The crude product was used in the next step without further purification. Intermediate 1-3 (100 mg, 369.49 μmol, 87.57% yield, 96.9% purity) was obtained as a yellow solid. LCMS (Method E): Rt=0.551 min, [M+H]⁺=263.1. ¹H NMR (400 MHz, METHANOL-d₄) δ=7.56-7.24 (m, 6H), 2.72 (m, 2H), 1.28 (m, 3H).

### Step 2: Synthesis of Intermediate 1-5

To a solution of Intermediate 1-3 (80 mg, 305.05 μmol, 1 eq) and Intermediate 1-4 (223.54 mg, 305.05 μmol, 1 eq) in DMF (1 mL) was added EDCI (175.44 mg, 915.15 μmol, 3 eq), NMM (154.28 mg, 1.53 mmol, 167.69 μL, 5 eq) and HOAt (41.52 mg, 305.05 μmol, 42.67 μL, 1 eq). The mixture was stirred at 25° C. for 1 hr. The reaction mixture was quenched by addition of water (5 mL) at 25° C., and then extracted with EA (5 mL*3). Then the organic layers were dried over Na₂SO₄, filtered and concentrated under reduced pressure to give a residue. The crude product was triturated with PE:EA=3:1 (5 mL) at 25° C. for 30 min to give Intermediate 1-5 (100 mg, 75.74 μmol, 24.83% yield, 74% purity) as a yellow solid. LCMS (Method E): Rt=0.611 min, [M+H]⁺=977.4.

### Step 3: Synthesis of I-479

To a solution of Intermediate 1-5 (50 mg, 51.18 μmol, 1 eq) in DMF (0.5 mL) was added piperidine (431.10 mg, 5.06 mmol, 0.5 mL, 98.93 eq). The mixture was stirred at 25° C. for 1 hr. The reaction mixture was concentrated under reduced pressure to give a residue. The residue was purified by prep-HPLC (column: Waters Xbridge 150*25 mm*5 um; mobile phase: [water (ammonia hydroxide v/v)-ACN]; gradient: 25%-55% B over 10 min). The target peak was concentrated and lyophilized to afford the desired product. I-479 (23 mg, 30.27 μmol, 59.16% yield, 99.35% purity) was obtained as a white solid. LCMS (Method E): Rt=0.484 min, [M+H]⁺=755.5. ¹H NMR (400 MHz, METHANOL-d₄) δ=8.39-8.32 (m, 1H), 7.97-7.90 (m, 1H), 7.90-7.77 (m, 2H), 7.52-7.41 (m, 3H), 7.39-7.23 (m, 5H), 7.13 (m, 1H), 4.36 (s, 2H), 3.81-3.49 (m, 12H), 3.49-3.42 (m, 2H), 3.37 (s, 1H), 3.23 (d, J=2.0 Hz, 1H), 2.80 (d, J=4.4 Hz, 2H), 2.75-2.65 (m, 2H), 1.30-1.22 (m, 3H).

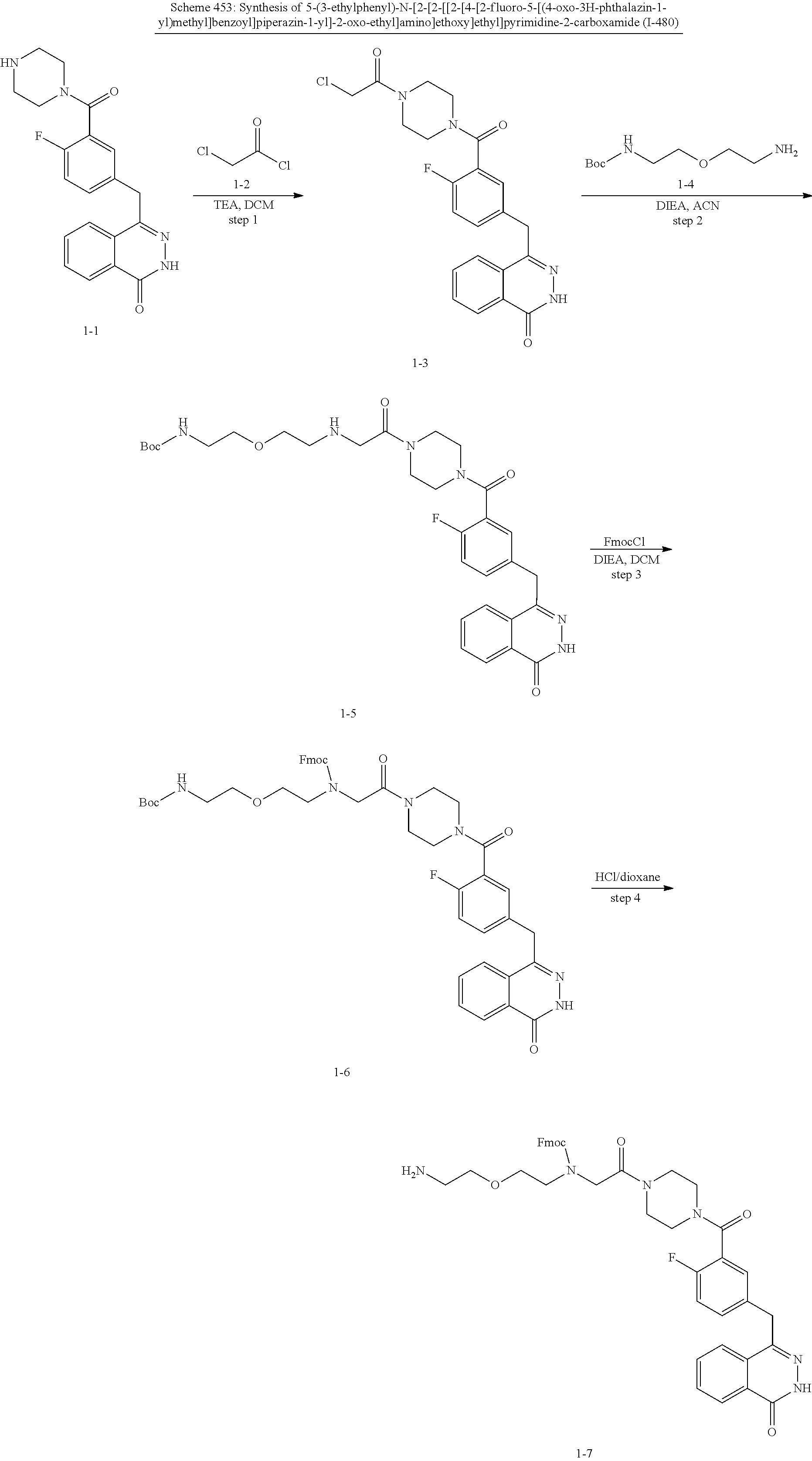

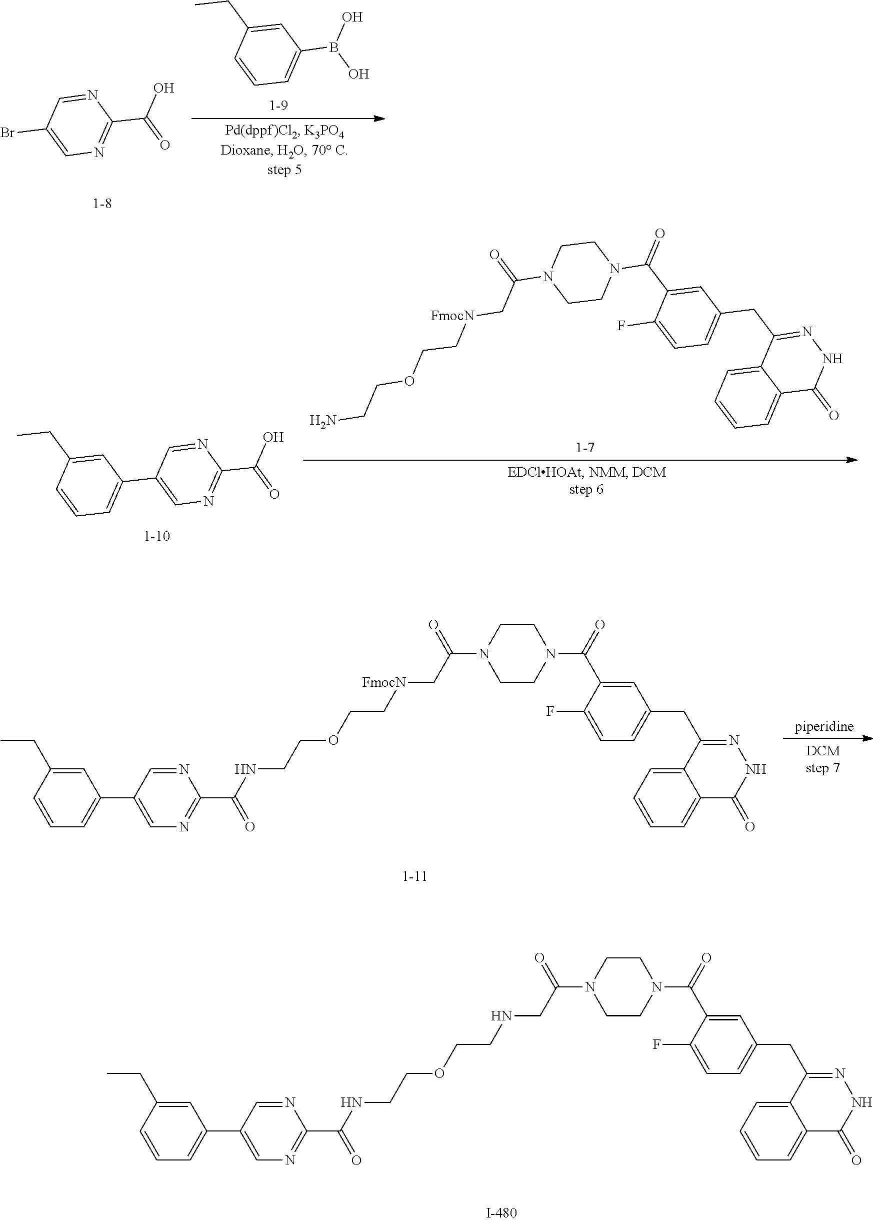

### Step 1: Synthesis of Intermediate 1-3

To a solution of Intermediate 1-1 (10 g, 27.29 mmol, 1 eq) and TEA (8.29 g, 81.88 mmol, 11.40 mL, 3 eq) in DCM (100 mL) was added Intermediate 1-2 (4.62 g, 40.94 mmol, 3.26 mL, 1.5 eq) at 0° C. Then the mixture was stirred at 0° C. for 1 hr. The reaction mixture was poured into water (200 mL) and extracted with DCM (200 mL*2), the organic layer was washed with brine (200 mL), dried over Na₂SO₄, and concentrated to give the crude product. Intermediate 1-3 (12 g, 26.15 mmol, 95.80% yield, 96.5% purity) was obtained as a yellow solid. LCMS (Method E): Rt=0.428 min, [M+H]⁺=443.2.

### Step 2: Synthesis of Intermediate 1-5

To a solution of Intermediate 1-3 (500 mg, 1.13 mmol, 1 eq) and DIEA (729.57 mg, 5.64 mmol, 983.25 μL, 5 eq) in DCM (5 mL) was added Intermediate 1-4 (345.92 mg, 1.69 mmol, 1.5 eq) at 25° C. Then the mixture was stirred at 25° C. for 1 hr. The reaction mixture was concentrated to give the crude product as a yellow oil. Intermediate 1-5 (680 mg, 730.47 μmol, 64.70% yield, 65.6% purity) was used for the next step without further purification. LCMS (Method G): Rt=0.515 min, [M+H]⁺=611.4.

### Step 3: Synthesis of Intermediate 1-6

To a solution of Intermediate 1-5 (680 mg, 1.11 mmol, 1 eq) and DIEA (431.74 mg, 3.34 mmol, 581.86 μL, 3 eq) in DCM (7 mL) was added 9H-fluoren-9-ylmethyl carbonochloridate (432.10 mg, 1.67 mmol, 1.5 eq) at 0° C. Then the mixture was stirred at 25° C. for 1 hr. The reaction mixture was poured into water (30 mL) and extracted with DCM (30 mL*2), the organic layer was washed with brine (30 mL), dried over Na₂SO₄, and concentrated to give the crude product. The crude product was purified by flash silica gel column chromatography (SiO₂, PE:EA=10:1 to 1:1) to give Intermediate 1-6 (400 mg, 466.32 μmol, 41.88% yield, 97.1% purity) as a yellow solid. LCMS (Method E): Rt=0.561 min, [M+H]⁺=833.4.

### Step 4: Synthesis of Intermediate 1-7

A solution of Intermediate 1-6 (400 mg, 480.24 μmol, 1 eq) in HCl/dioxane (4 mL) was stirred at 25° C. for 1 hr. The reaction mixture was concentrated to give the crude product. The crude product was triturated with EA (100 mL) at 25° C. for 30 min to give Intermediate 1-7 (350 mg, 445.42 μmol, 92.75% yield, 97.9% purity, HCl salt) as an off-white solid. LCMS (Method E): Rt=0.466 min, [M+H]⁺=733.4.

### Step 5: Synthesis of Intermediate 1-10

To a solution of Intermediate 1-8 (500 mg, 2.46 mmol, 1 eq), Intermediate 1-9 (554.14 mg, 3.69 mmol, 1.5 eq), and K₃PO₄ (1.57 g, 7.39 mmol, 3 eq) in dioxane (5 mL) and H₂O (0.5 mL) was added ditert-butyl(cyclopentyl)phosphane;dichloropalladium;iron (160.53 mg, 246.31 μmol, 0.1 eq). Then the mixture was stirred at 70° C. for 1 hr under N₂. The reaction mixture was poured into water (20 mL) and extracted with EA (20 mL*2), the organic layer was washed with brine (20 mL) and dried over Na₂SO₄, and concentrated to give the crude product. The crude product was purified by flash silica gel chromatography (SiO₂, PE:EA=3:1 to 1:1) to give Intermediate 1-10 (300 mg, 1.04 mmol, 42.21% yield, 79.1% purity) as an off-white solid. LCMS (Method E): Rt=0.454 min, [M+H]⁺=229.1.

### Step 6: Synthesis of Intermediate 1-11

To a solution of Intermediate 1-10 (80 mg, 350.50 μmol, 1 eq), EDCI (134.38 mg, 701.00 μmol, 2 eq), HOAt (23.85 mg, 175.25 μmol, 24.52 μL, 0.5 eq), and NMM (177.26 mg, 1.75 mmol, 192.67 μL, 5 eq) in DCM (1 mL) was added Intermediate 1-7 (269.62 mg, 350.50 μmol, 1 eq, HCl salt). Then the mixture was stirred at 25° C. for 1 hr. The reaction mixture was poured into water (30 mL) and extracted with DCM (30 mL*2), the organic layer was washed with brine (30 mL) and dried over Na₂SO₄, and concentrated to give the crude product. The crude product was triturated with PE:EA=3:1 (5 mL) at 25° C. for 30 min to give Intermediate 1-11 (120 mg, 126.22 μmol, 36.01% yield, 99.19% purity) as a colorless oil. LCMS (Method E): Rt=0.568 min, [M+H]⁺=943.3.

### Step 7: Synthesis of I-480

To a solution of Intermediate 1-11 (120 mg, 127.25 μmol, 1 eq) in DCM (2 mL) was added piperidine (54.17 mg, 636.25 μmol, 62.83 μL, 5 eq). Then the mixture was stirred at 25° C. for 1 hr. The reaction mixture was filtered to give the filtrate. The filtrate was purified by reverse phase chromatography (basic method) to give I-480 (24.04 mg, 33.35 μmol, 26.21% yield, 100% purity) as a white solid. LCMS (Method E): Rt=0.457 min, [M+H]⁺=721.2. ¹H NMR (400 MHz, DMSO-d₆) δ=13.00-12.25 (m, 1H), 9.26 (br d, J=3.2 Hz, 2H), 8.89 (br s, 1H), 8.35-8.17 (m, 1H), 8.02-7.93 (m, 1H), 7.93-7.80 (m, 2H), 7.72 (s, 1H), 7.68 (br d, J=8.0 Hz, 1H), 7.47-7.45 (m, 2H), 7.36 (br s, 2H), 7.24-7.22 (m, 1H), 4.33 (s, 2H), 3.67-3.38 (m, 14H), 3.20-3.12 (m, 2H), 2.72-2.64 (m, 4H), 1.42 (br d, J=3.2 Hz, 1H), 1.30-1.18 (m, 3H).

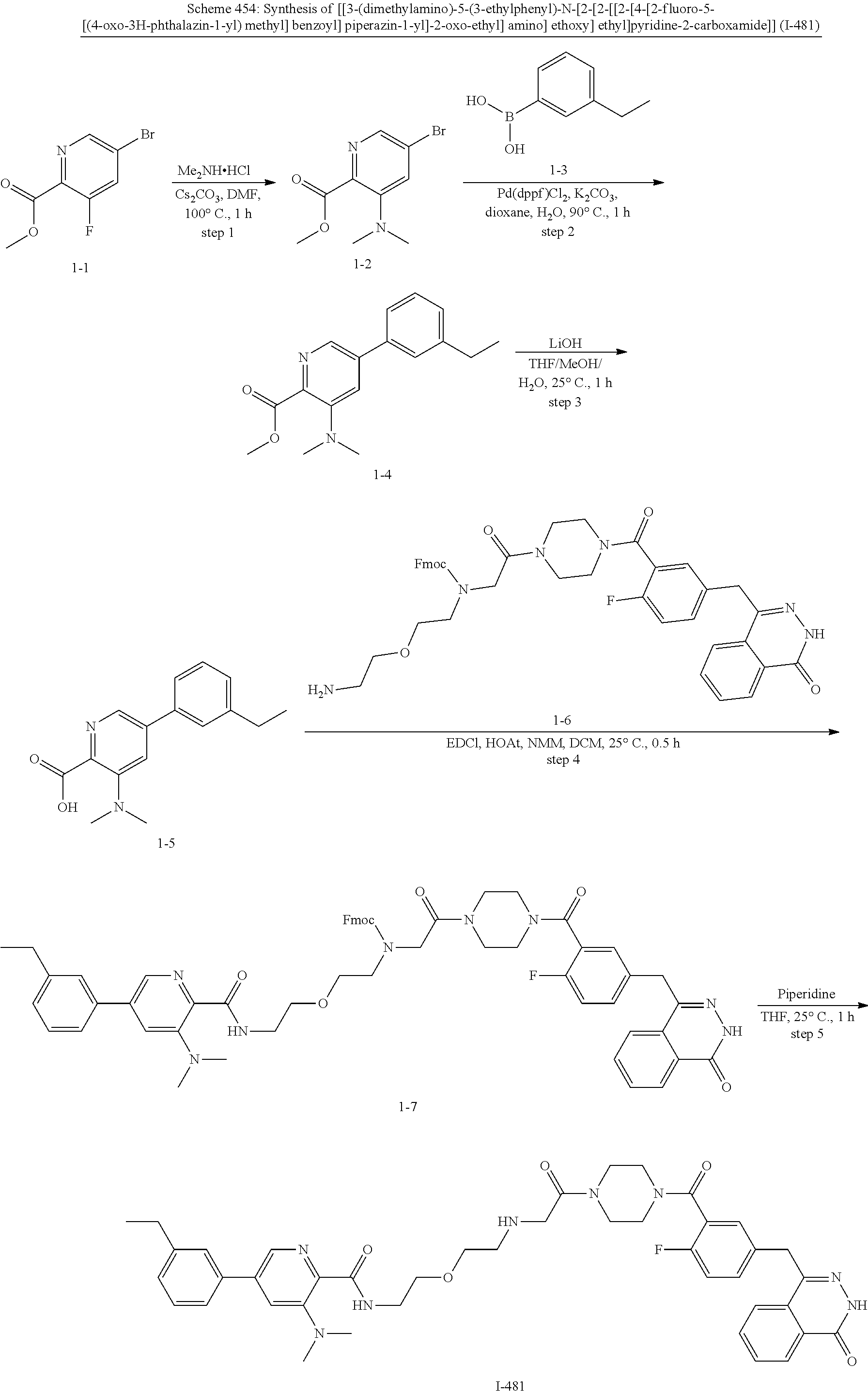

### Step 1: Synthesis of Intermediate 1-2

To a solution of intermediate 1-1 (200 mg, 854.62 μmol, 1 eq) in DMF (3 mL) was added Me₂NH (96.32 mg, 854.62 μmol, 108.23 μL, 1 eq, 40% aq.) and Cs₂CO₃ (835.35 mg, 2.56 mmol, 3 eq). The mixture was stirred at 100° C. for 1 hr. The reaction mixture was diluted with water (20 mL) and extracted with EA (20 mL*3). The combined organic layers were washed with brine (20 mL*3) and dried over sodium sulfate, filtered and concentrated under reduced pressure to give intermediate 1-2 (200 mg, 770.04 μmol, 90.10% yield) as a yellow oil. LCMS (Method D): Rt=0.320 min, [M+H]⁺=260.9.

### Step 2: Synthesis of Intermediate 1-4

To a solution of intermediate 1-2 (200 mg, 771.90 μmol, 1 eq) and intermediate 1-3 (115.77 mg, 771.90 μmol, 1 eq) in H₂O (0.6 mL) and dioxane (2 mL) was added Pd(dtbpf)Cl₂ (56.48 mg, 77.19 μmol, 0.1 eq) and dipotassium; carbonate (320.04 mg, 2.32 mmol, 3 eq). The mixture was stirred at 90° C. for 1 hr under N₂. The reaction mixture was diluted with water (20 mL) and extracted with EA (20 mL*3). The combined organic layers were washed with brine (20 mL*3) and dried over sodium sulfate, filtered and concentrated under reduced pressure to give a residue. The residue was purified by flash silica gel chromatography (ISCO; 12 g SepaFlash Silica Flash Column, Eluent of 0˜10% Methanol: Ethyl acetate gradient @40 mL/min). The eluent was concentrated under reduced pressure to give intermediate 1-4 (160 mg, 553.87 μmol, 71.75% yield, 98.433% purity) as a yellow oil. LCMS (Method D): Rt=0.398 min, [M+H]⁺=285.0.

### Step 3: Synthesis of Intermediate 1-5

To a solution of intermediate 1-4 (160 mg, 562.68 μmol, 1 eq) in THF (0.6 mL), H₂O (0.15 mL) and MeOH (0.6 mL) was added LiOH H₂O (70.84 mg, 1.69 mmol, 3 eq). The mixture was stirred at 25° C. for 1 hr. The pH of the reaction solution was adjusted to 3-4 with citric acid monohydrate. Then the mixture was diluted with water (10 mL) and extracted with EA (10 mL*3). The combined organic layers were washed with brine (10 mL*3) and dried over sodium sulfate, filtered and concentrated under reduced pressure to give intermediate 1-5 (140 mg, 510.12 μmol, 90.66% yield, 98.498% purity) as a yellow oil. LCMS (Method D): Rt=0.315 min, [M+H]⁺=271.0.

### Step 4: Synthesis of Intermediate 1-7

A solution of intermediate 1-5 (40 mg, 147.97 μmol, 1 eq) in DCM (4 mL) was added EDCI (85.10 mg, 443.91 μmol, 3 eq), NMM (74.83 mg, 739.85 μmol, 81.34 μL, 5 eq) and HOAt (20.14 mg, 147.97 μmol, 20.70 μL, 1 eq) was stirred at 25° C. for 0.5 hr. Then intermediate 1-6 (108.43 mg, 140.96 μmol, 9.53e−1 eq, HCl salt) was added into the mixture and it stirred at 25° C. for 0.5 hr. The reaction mixture was diluted with water (10 mL) and extracted with DCM (10 mL*3). The combined organic layers were washed with brine (10 mL*3) and dried over sodium sulfate, filtered and concentrated under reduced pressure to give intermediate 1-7 (120 mg, 121.81 μmol, 82.32% yield) as a yellow oil. LCMS (Method D): Rt=0.444 min, [M+H]⁺=985.4.

### Step 5: Synthesis of I-481

To a solution of intermediate 1-7 (110 mg, 111.66 μmol, 1 eq) in THF (1.5 mL) was added piperidine (163.15 mg, 1.92 mmol, 189.23 μL, 17.16 eq). The mixture was stirred at 25° C. for 1 hr. The reaction mixture was diluted with water (10 mL) and extracted with EA (10 mL*3). The combined organic layers were washed with brine (10 mL*3) and dried over sodium sulfate, filtered and concentrated under reduced pressure to give a residue. The residue was purified by reversed-phase chromatography (neutral condition). The eluent was concentrated under reduced pressure to remove ACN and then lyophilized to give I-481 (33.74 mg, 43.71 μmol, 39.15% yield, 98.834% purity) as a white solid. LCMS (Method D): Rt=0.331 min, [M+H]⁺=763.2. ¹H NMR (400 MHz, METHANOL-d4) δ=8.40-8.33 (m, 1H), 8.26 (s, 1H), 7.95-7.90 (m, 1H), 7.90-7.79 (m, 2H), 7.59 (d, J=1.6 Hz, 1H), 7.51-7.41 (m, 3H), 7.41-7.30 (m, 2H), 7.29-7.22 (m, 1H), 7.13 (s, 1H), 4.68-4.51 (m, 1H), 4.36 (s, 2H), 3.74-3.59 (m, 9H), 3.55-3.50 (m, 2H), 3.49-3.42 (m, 2H), 3.36 (s, 1H), 3.25-3.18 (m, 1H), 2.96-2.89 (m, 6H), 2.80 (d, J=4.4 Hz, 2H), 2.77-2.65 (m, 2H), 1.33-1.20 (m, 3H). ¹⁹F NMR (377 MHz, METHANOL-d4) δ=−120.674.

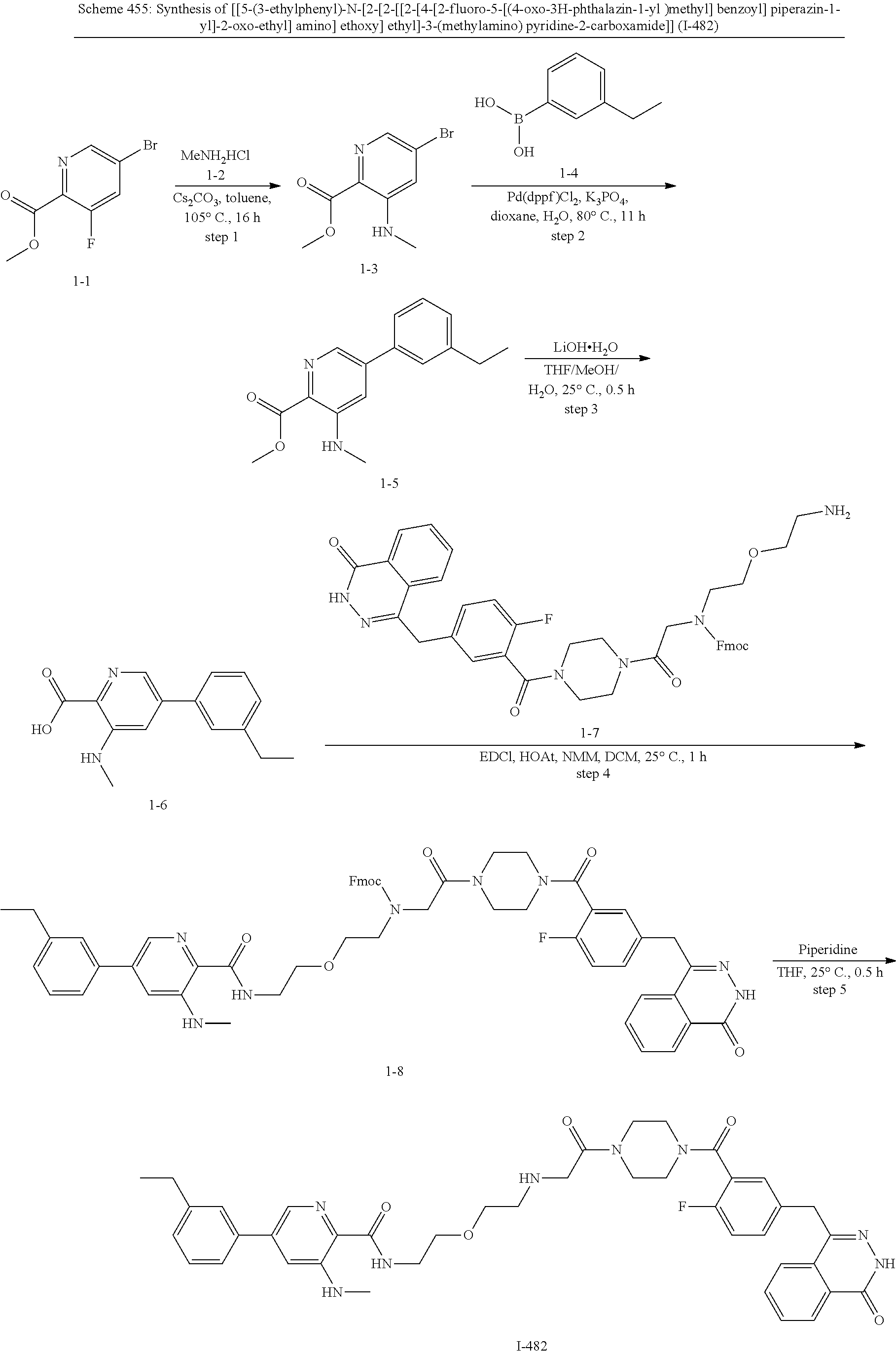

### Step 1: Synthesis of Intermediate 1-3

To a solution of intermediate 1-1 (500 mg, 2.14 mmol, 1 eq) and intermediate 1-2 (577.02 mg, 8.55 mmol, 4 eq) in toluene (5 mL) was added Cs₂CO₃ (2.78 g, 8.55 mmol, 4 eq). Then the mixture was stirred at 105° C. for 16 hr. The reaction mixture was concentrated under reduced pressure to give a residue. The residue was purified by flash silica gel chromatography (ISCO; 12 g SepaFlash Silica Flash Column, Eluent of 0˜30% EA/PE ether gradient @80 mL/min). The eluent was concentrated under reduced pressure to give the product. Intermediate 1-3 (200 mg, 816.08 μmol, 38.20% yield) was obtained as a white solid.

### Step 2: Synthesis of Intermediate 1-5

To a solution of intermediate 1-3 (200 mg, 816.08 μmol, 1 eq) and intermediate 1-4 (244.80 mg, 1.63 mmol, 2 eq) in dioxane (2 mL) and H₂O (0.4 mL) was added K₃PO₄ (173.23 mg, 816.08 μmol, 1 eq) and Pd(dtbpf)Cl₂ (53.19 mg, 81.61 μmol, 0.1 eq). The mixture was stirred at 80° C. for 1 hr under N₂. The reaction mixture was diluted with water (80 mL) and extracted with EA (30 mL*2). The combined organic layers were concentrated under reduced pressure to give a residue. The residue was purified by flash silica gel chromatography (ISCO; 20 g SepaFlash Silica Flash Column, Eluent of 0˜50% EA/PE ether gradient @60 mL/min). The eluent was concentrated under reduced pressure to give the product. Intermediate 1-5 (100 mg, 369.92 μmol, 45.33% yield) was obtained as a white solid.

### Step 3: Synthesis of Intermediate 1-6

To a solution of intermediate 1-5 (100 mg, 369.92 μmol, 1 eq) in THF (1 mL), MeOH (0.5 mL) and H₂O (0.3 mL) was added LiOH H₂O (15.52 mg, 369.92 μmol, 1 eq). The mixture was stirred at 25° C. for 0.5 hr. The reaction mixture was concentrated under reduced pressure to remove THF, 1 N HCl was added to adjust the pH to 3-4, and then the mixture was extracted with EA (10 mL*3). The combined organic layers were concentrated under reduced pressure to give a product. Intermediate 1-6 (90 mg, 351.15 μmol, 94.93% yield) was obtained as a white solid. LCMS (Method D): Rt=0.384 min, [M+H]+=256.9.

### Step 4: Synthesis of Intermediate 1-8

To a solution of intermediate 1-6 (74.96 mg, 292.49 μmol, 1.5 eq) and intermediate 1-7 (150 mg, 194.99 μmol, 1 eq, HCl salt) in DMF (2 mL) was added EDCI (112.14 mg, 584.98 μmol, 3 eq), HOAt (26.54 mg, 194.99 μmol, 27.28 μL, 1 eq), and NMM (98.61 mg, 974.96 μmol, 107.19 μL, 5 eq). The mixture was stirred at 25° C. for 1 hr. The reaction mixture was diluted with water (40 mL) and extracted with EA (2 mL*3). The combined organic layers were concentrated under reduced pressure to give a residue. The residue was purified by reversed phase chromatography (0.1% FA). The eluent was concentrated under reduced pressure to remove MeCN and then lyophilized to give the product. Intermediate 1-8 (70 mg, 72.08 μmol, 36.97% yield) was obtained as a white solid. LCMS (Method D): Rt=0.555 min, [M+H]⁺=971.3

### Step 5: Synthesis of I-482

To a solution of intermediate 1-8 (60 mg, 61.79 μmol, 1 eq) in THF (0.48 mL) was added piperidine (0.06 mL) and then the mixture was stirred at 25° C. for 0.5 hr. The reaction mixture was concentrated under reduced pressure to give a residue. The residue was purified by reversed phase chromatography (0.1% FA). The eluent was concentrated under reduced pressure to remove ACN and then lyophilized to give the product. I-482 (17.82 mg, 22.32 μmol, 36.12% yield, 99.55% purity, FA) was obtained as a white solid. LCMS (Method D): Rt=0.406 min, [M+H]⁺=749.3. ¹H NMR (400 MHz, METHANOL-d4) δ=8.64-8.47 (m, 1H), 8.36 (d, J=7.6 Hz, 1H), 8.01 (d, J=16.4 Hz, 1H), 7.96-7.90 (m, 1H), 7.89-7.79 (m, 2H), 7.52-7.41 (m, 3H), 7.37 (d, J=7.6 Hz, 1H), 7.31 (d, J=2.0 Hz, 1H), 7.29-7.20 (m, 2H), 7.18-7.08 (m, 1H), 4.35 (d, J=4.0 Hz, 2H), 3.83-3.54 (m, 12H), 3.47 (s, 2H), 3.28-3.19 (m, 2H), 3.04-2.89 (m, 5H), 2.76-2.64 (m, 2H), 1.31-1.20 (m, 3H). ¹⁹F NMR (377 MHz, METHANOL-d4) δ=−120.605.

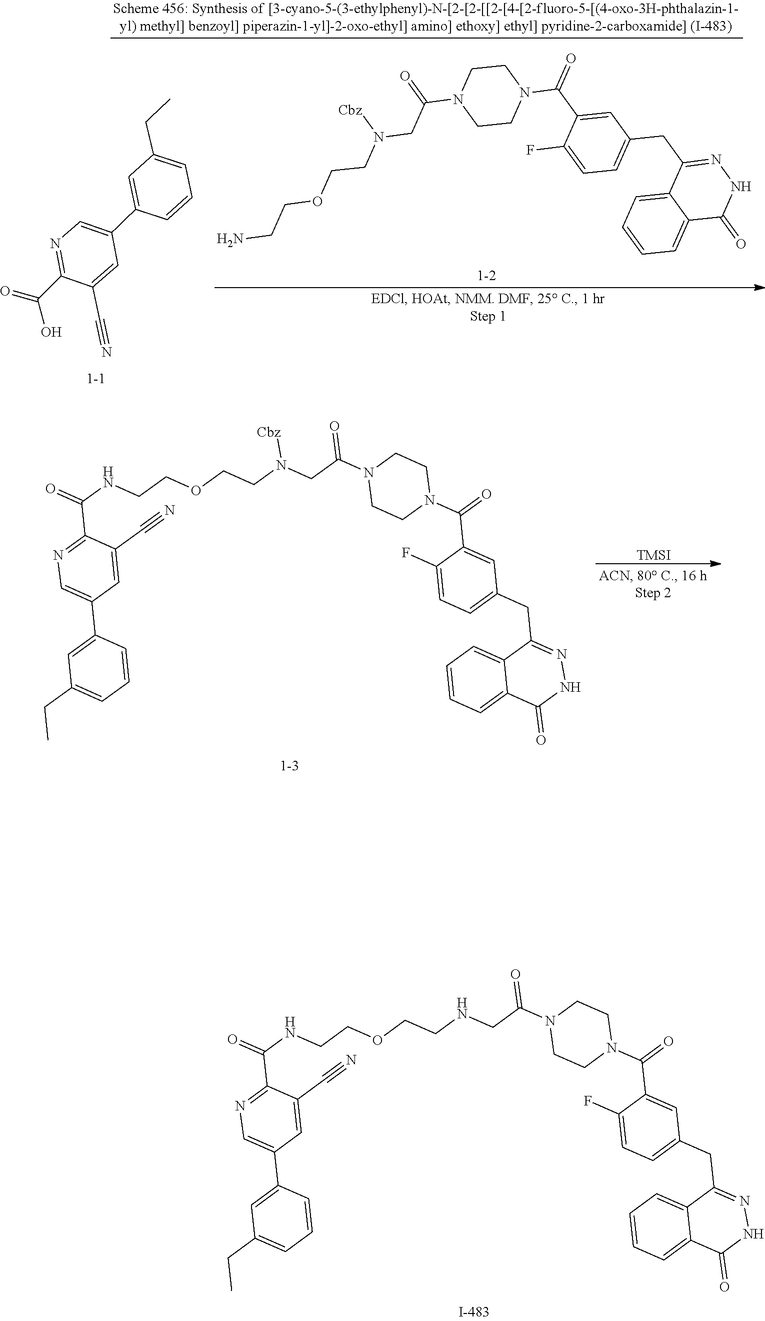

### Step 1: Synthesis of Intermediate 1-3.

To a solution of intermediate 1-1 (18.52 mg, 73.41 μmol, 1 eq) in DMF (0.5 mL) was added intermediate 1-2 (50 mg, 73.41 μmol, 1 eq, HCl salt), EDCI (42.22 mg, 220.22 μmol, 3 eq), HOAt (9.99 mg, 73.41 μmol, 10.27 μL, 1 eq) and NMM (37.12 mg, 367.03 μmol, 40.35 μL, 5 eq). The mixture was stirred at 25° C. for 1 hr. The reaction mixture was poured into H₂O (5 mL), and extracted with EA (2 mL*3). The combined organic layers were dried over Na₂SO₄, filtered and concentrated under reduced pressure to give a residue. The residue was purified by column chromatography (SiO₂, EA/MeOH=1/0 to 7/3) and concentrated to give the product. Intermediate 1-3 (70 mg, crude) was obtained as a yellow solid. LCMS (Method D): Rt=0.458 min, [M+H]⁺=879.4.

### Step 2: Synthesis of I-483

To a mixture of intermediate 1-3 (60 mg, 68.26 μmol, 1 eq) in ACN (0.5 mL) was added TMSI (13.66 mg, 68.26 μmol, 9.29 μL, 1 eq) and the mixture was stirred at 80° C. for 16 h. The reaction mixture was concentrated to give a residue. The residue was purified by prep-HPLC (column: CD04-Welch Utimate C18 150*25*7 um; mobile phase: [water (FA)-ACN]; gradient: 20%-50% B over 8 min) and lyophilized to give the product. I-483 (10.01 mg, 12.95 μmol, 18.97% yield, 96.363% purity) was obtained as a white solid. LCMS (Method D): Rt=0.423 min, [M+H]⁺=745.3.

¹H NMR (400 MHz, METHANOL-d4) δ=9.09 (s, 1H), 8.58-8.51 (m, 1H), 8.40-8.26 (m, 1H), 7.98-7.90 (m, 1H), 7.90-7.76 (m, 2H), 7.64-7.53 (m, 2H), 7.52-7.40 (m, 2H), 7.40-7.28 (m, 2H), 7.19-7.10 (m, 1H), 4.38 (s, 2H), 3.81-3.75 (m, 2H), 3.75-3.63 (m, 8H), 3.58-3.35 (m, 4H), 3.29-3.22 (m, 2H), 3.02-2.88 (m, 2H), 2.81-2.67 (m, 2H), 1.33-1.23 (m, 3H). ¹⁹F NMR (377 MHz, METHANOL-d₄), δ=120.71.

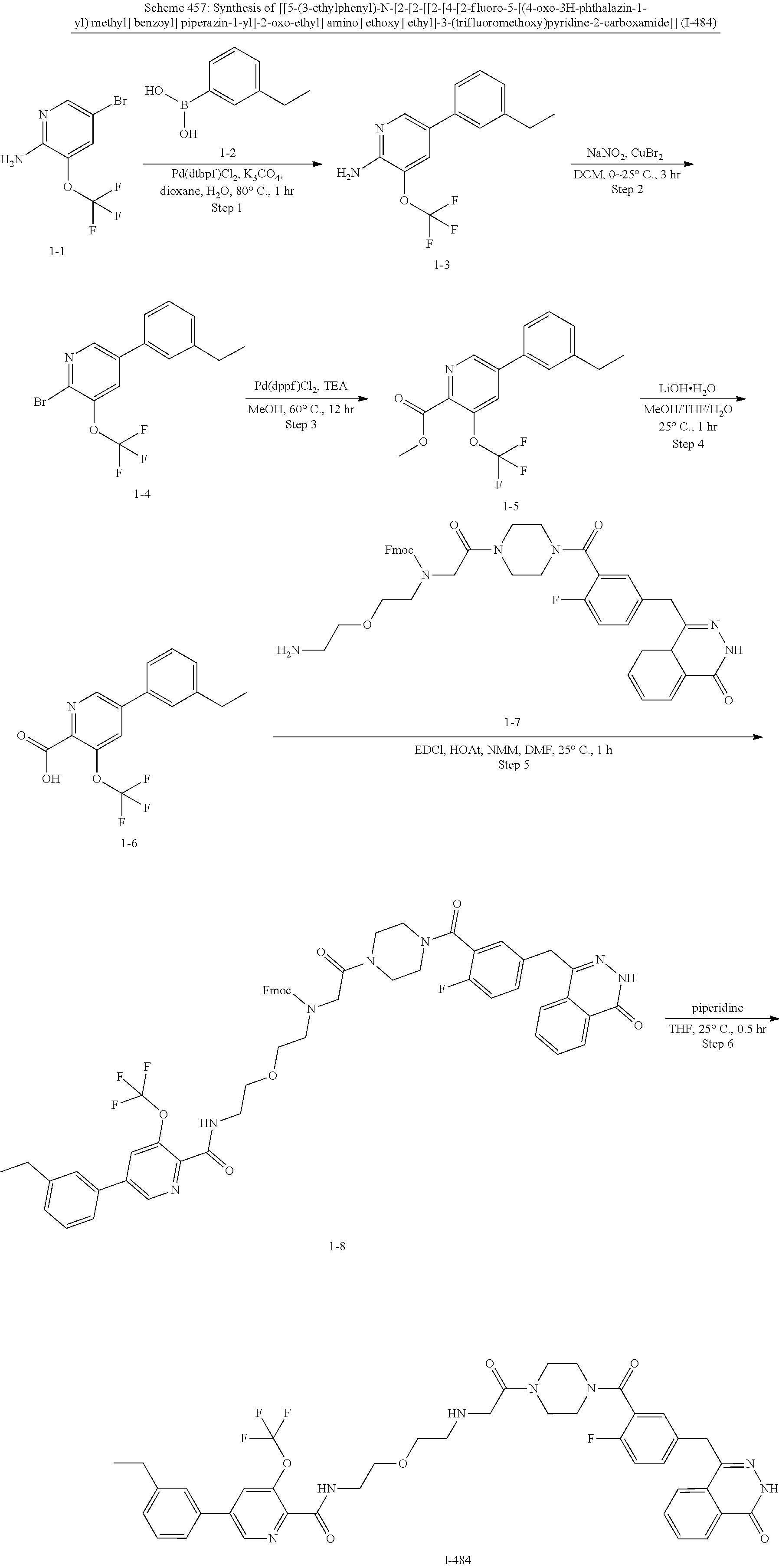

### Step 1: Synthesis of Intermediate 1-3

To a solution of intermediate 1-1 (500 mg, 1.95 mmol, 1 eq) and Intermediate 1-2 (320.96 mg, 2.14 mmol, 1.1 eq) in dioxane (5 mL) and H₂O (1 mL) was added Pd(dtbpf)Cl₂ (126.79 mg, 194.55 μmol, 0.1 eq) and K₃PO₄ (1.24 g, 5.84 mmol, 3 eq). The mixture was stirred at 80° C. for 1 hr under N₂ atmosphere. The reaction mixture was diluted with H₂O (5 mL) and extracted with solvent EA (5 mL*2). The combined organic layers were dried over Na₂SO₄, filtered and concentrated under reduced pressure to give a residue. The residue was purified by flash silica gel chromatography (ISCO; 20 g SepaFlash Silica Flash Column, Eluent of 0-15% Ethyl acetate/Petroleum ether gradient @60 mL/min). The eluent was concentrated under reduced pressure to give intermediate 1-3 (470 mg, 1.67 mmol, 85.59% yield, 100% purity) as a yellow oil. LCMS (Method D): Rt=0.408 min, [M+H]⁺=283.1. ¹H NMR (400 MHz, METHANOL-d4) δ=8.18 (d, J=2.0 Hz, 1H), 7.70 (s, 1H), 7.37-7.31 (m, 3H), 7.21-7.16 (m, 1H), 2.74-2.66 (m, 2H), 1.30-1.23 (m, 3H). ¹⁹F NMR (376 MHz, METHANOL-d4) δ=−59.744.

### Step 2: Synthesis of Intermediate 1-4

To a solution of Intermediate 1-3 (450 mg, 1.59 mmol, 1 eq) in DCM (5 mL) was added NaNO₂ (329.99 mg, 4.78 mmol, 3 eq) and CuBr₂ (356.09 mg, 1.59 mmol, 74.65 μL, 1 eq). The mixture was stirred at 0° C. for 1 hr. Then the mixture was stirred at 25° C. for 2 hr. The mixture was filtered and the filtrate was purified by flash silica gel chromatography (ISCO; 20 g SepaFlash Silica Flash Column, Eluent of 0˜12% EA/PE ether gradient @60 mL/min). The eluent was concentrated under reduced pressure to give intermediate 1-4 (260 mg, 738.37 μmol, 46.31% yield, 98.3% purity) as a yellow oil. LCMS (Method D): Rt=0.551 min, [M+H]⁺=345.9.

### Step 3: Synthesis of Intermediate 1-5

To a solution of intermediate 1-4 (130 mg, 375.57 μmol, 1 eq) in MeOH (3 mL) was added TEA (114.01 mg, 1.13 mmol, 156.82 μL, 3 eq) and Pd(dppf)Cl₂ (27.48 mg, 37.56 μmol, 0.1 eq) under N₂ atmosphere. The suspension was degassed and purged with CO 3 times. The mixture was stirred under CO (50 Psi) at 60° C. for 12 hr. The mixture was filtered and concentrated in vacuum. The residue was purified by flash silica gel chromatography (ISCO; 12 g SepaFlash Silica Flash Column, Eluent of 0˜17% EA/PE ether gradient @60 mL/min). The eluent was concentrated under reduced pressure to give intermediate 1-5 (110 mg, 335.12 μmol, 89.23% yield, 99.1% purity) as a yellow oil. LCMS (Method D): Rt=0.498 min, [M+H]⁺=326.0.

### Step 4: Synthesis of Intermediate 1-6

To a solution of intermediate 1-5 (110 mg, 338.17 μmol, 1 eq) in MeOH (0.5 mL), THF (0.5 mL) and H₂O (0.1 mL) was added LiOH H₂O (42.57 mg, 1.01 mmol, 3 eq). The mixture was stirred at 25° C. for 1 hr. The mixture was diluted with H₂O (1 mL) and HCl (1M) was added in to adjust pH to 4, then the mixture was extracted with EA (1 mL*2). The combined organic layers were dried over Na₂SO₄, filtered and concentrated under reduced pressure to give intermediate 1-6 (100 mg, 311.32 μmol, 92.06% yield, 96.9% purity) as a white solid, which was used in the next step without further purification. LCMS (Method D): Rt=0.448 min, [M+H]⁺=312.0. ¹H NMR (400 MHz, METHANOL-d4) δ=8.48 (s, 1H), 8.94-8.87 (m, 1H), 8.12 (s, 1H), 7.63-7.51 (m, 2H), 7.50-7.43 (m, 1H), 7.39-7.32 (m, 1H), 2.81-2.71 (m, 2H), 1.30 (t, J=7.6 Hz, 3H). ¹⁹F NMR (377 MHz, METHANOL-d4) δ=−59.076.

### Step 5: Synthesis of Intermediate 1-8

To a solution of intermediate 1-7 (80 mg, 104.00 μmol, 1 eq, HCl salt) in DMF (1 mL) was added EDCI (59.81 mg, 311.99 μmol, 3 eq), HOAt (14.16 mg, 104.00 μmol, 14.55 μL, 1 eq), intermediate 1-6 (32.37 mg, 104.00 μmol, 1 eq) and NMM (52.59 mg, 519.98 μmol, 57.17 μL, 5 eq). The mixture was stirred at 25° C. for 1 hr. The mixture was diluted with H₂O (5 mL) and extracted with EA (5 mL*2). The combined organic layers were washed with brine (5 mL), dried over Na₂SO4, filtered and concentrated under reduced pressure to give intermediate 1-8 (160 mg, crude) as a yellow oil, which was used for the next step without further purification. LCMS (Method D): Rt=0.551 min, [M/2+H]⁺=513.9.

### Step 6: Synthesis of I-484

A solution of intermediate 1-8 (130 mg, 126.70 μmol, 1 eq) in THF (1.6 mL) and piperidine (172.44 mg, 2.03 mmol, 0.2 mL, 15.98 eq) was stirred at 25° C. for 0.5 hr. The mixture was filtered, then the filtrate was purified by reversed phase chromatography (0.1% FA). The eluent was concentrated under reduced pressure to remove ACN and then lyophilized to give I-484 (27.28 mg, 31.75 μmol, 25.06% yield, 98.9% purity, FA salt) as a yellow solid. LCMS (Method D): Rt=0.402 min, [M+H]⁺=804.2. ¹H NMR (400 MHz, METHANOL-d4) δ=8.88 (d, J=4.8 Hz, 1H), 8.49 (s, 1H), 8.36 (d, J=7.6 Hz, 1H), 8.08 (s, 1H), 7.96-7.91 (m, 1H), 7.89-7.79 (m, 2H), 7.57-7.42 (m, 4H), 7.39-7.30 (m, 2H), 7.19-7.10 (m, 1H), 4.40-4.35 (m, 2H), 3.97 (s, 1H), 3.89 (s, 1H), 3.79-3.63 (m, 9H), 3.55-3.47 (m, 2H), 3.40-3.32 (m, 2H), 3.27 (s, 1H), 3.17-3.09 (m, 2H), 2.78-2.69 (m, 2H), 1.32-1.23 (m, 3H). ¹⁹F NMR (377 MHz, METHANOL-d4) δ=−58.896, −120.678.

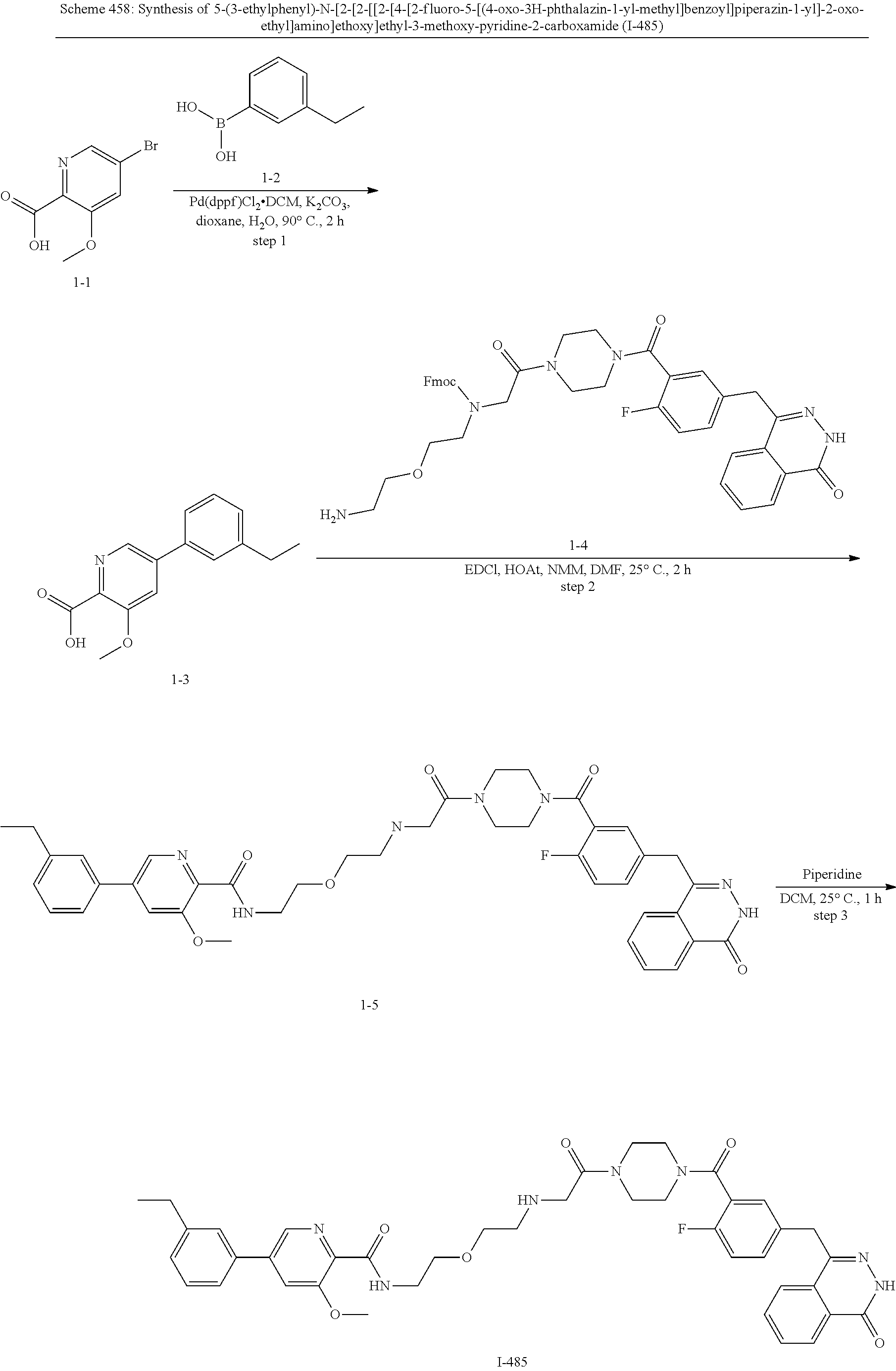

### Step 1: Synthesis of Intermediate 1-3

To a solution of Intermediate 1-1 (200 mg, 861.95 μmol, 1 eq) and Intermediate 1-2 (258.56 mg, 1.72 mmol, 2 eq) in dioxane (2 mL) and H₂O (0.2 mL) was added Pd(dppf)Cl₂·CH₂Cl₂ (70.39 mg, 86.20 μmol, 0.1 eq) and K₂CO₃ (357.38 mg, 2.59 mmol, 3 eq). The mixture was stirred at 90° C. for 2 hr. The mixture was poured into 10 mL of water and acidified to pH=3 with 1N HCl, and the mixture was then extracted with EA (20 mL*2). The combined organic phase was dried over anhydrous Na₂SO₄, filtered and concentrated in vacuum. The crude product was purified by reversed-phase HPLC (0.1% FA condition). Intermediate 1-3 (120 mg, 466.41 μmol, 54.11% yield) was obtained as yellow solid. LCMS (Method E): Rt=0.453 min, [M+H]⁺=258.0. ¹H NMR (400 MHz, CHLOROFORM-d) δ=8.51-8.28 (m, 1H), 7.97-7.74 (m, 1H), 7.48-7.38 (m, 4H), 4.43-4.10 (m, 3H), 2.68-2.54 (m, 2H), 1.35-1.27 (m, 3H).

### Step 2: Synthesis of Intermediate 1-5

To a solution of Intermediate 1-3 (53.51 mg, 207.99 μmol, 2 eq) in DMF (2 mL) was added HOAt (56.62 mg, 415.99 μmol, 58.19 μL, 4 eq) and EDCI (79.74 mg, 415.99 μmol, 4 eq). The mixture was stirred at 25° C. for 30 min. Then Intermediate 1-4 (80 mg, 104.00 μmol, 1 eq, HCl salt) and NMM (63.11 mg, 623.98 μmol, 68.60 μL, 6 eq) were added and the mixture was stirred at 25° C. for 2 hr. The mixture was poured into 20 mL of water and extracted with EA (20 mL*2). The combined organic phase was dried over anhydrous Na₂SO₄, filtered and concentrated in vacuum. The residue was purified by flash silica gel chromatography (ISCO; 20 g SepaFlash Silica Flash Column, Eluent of 0˜30% MeOH/EA @20 mL/min). Intermediate 1-5 (70 mg, 72.01 μmol, 69.24% yield) was obtained as a yellow solid. LCMS (Method E): Rt=0.562 min, [M+H]⁺=972.4. ¹H NMR (400 MHz, CHLOROFORM-d) δ=8.46 (br d, J=7.9 Hz, 1H), 8.43-8.32 (m, 1H), 8.03-7.89 (m, 1H), 7.80-7.65 (m, 5H), 7.58 (br d, J=7.8 Hz, 1H), 7.54-7.50 (m, 1H), 7.46-7.28 (m, 11H), 7.11-6.87 (m, 1H), 4.62-4.44 (m, 2H), 4.32-4.17 (m, 4H), 4.05-3.85 (m, 5H), 3.76-3.57 (m, 7H), 3.53 (br s, 2H), 3.48-3.37 (m, 4H), 3.34-3.17 (m, 3H), 2.80-2.64 (m, 3H), 2.05 (s, 3H), 1.33-1.29 (m, 3H).

### Step 3: Synthesis of I-485

To a solution of Intermediate 1-5 (70 mg, 72.01 μmol, 1 eq) in DCM (2 mL) was added piperidine (30.66 mg, 360.06 μmol, 35.56 μL, 5 eq). The mixture was stirred at 25° C. for 1 hr. The mixture was concentrated in vacuum. The crude product was purified by reversed-phase HPLC (0.1% FA condition). I-485 (14.94 mg, 18.77 μmol, 26.07% yield, 100% purity, FA salt) was obtained as off-white solid. LCMS (Method E): Rt=0.465 min, [M+H]⁺=750.3. SFC: Rt=2.736 min, 3.407 min.

¹H NMR (400 MHz, METHANOL-d₄) δ=8.48 (s, 1H), 8.43 (br d, J=13.3 Hz, 1H), 8.36 (d, J=7.8 Hz, 1H), 7.96-7.90 (m, 1H), 7.89-7.74 (m, 3H), 7.57-7.45 (m, 3H), 7.44-7.37 (m, 1H), 7.36-7.27 (m, 2H), 7.18-7.08 (m, 1H), 4.36 (br d, J=2.4 Hz, 2H), 4.02 (br d, J=11.3 Hz, 3H), 4.00-3.83 (m, 2H), 3.82-3.57 (m, 9H), 3.49 (br d, J=5.1 Hz, 2H), 3.34 (br s, 2H), 3.28-3.21 (m, 1H), 3.19-3.11 (m, 2H), 2.82-2.64 (m, 2H), 1.27 (td, J=7.6, 11.4 Hz, 3H). ¹⁹F NMR (377 MHz, METHANOL-d₄) δ=−120.66 (br d, J=6.7 Hz, 1F).

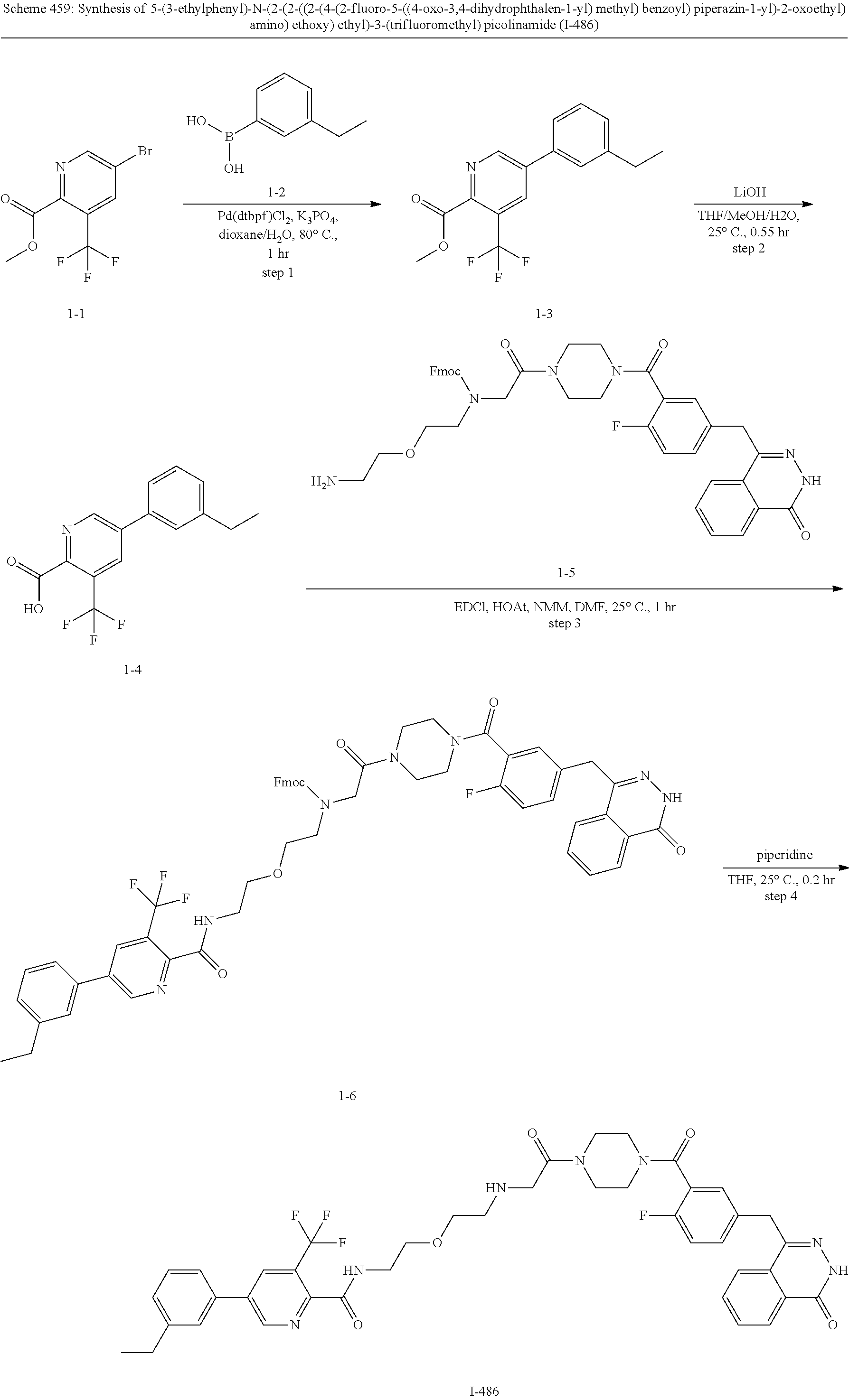

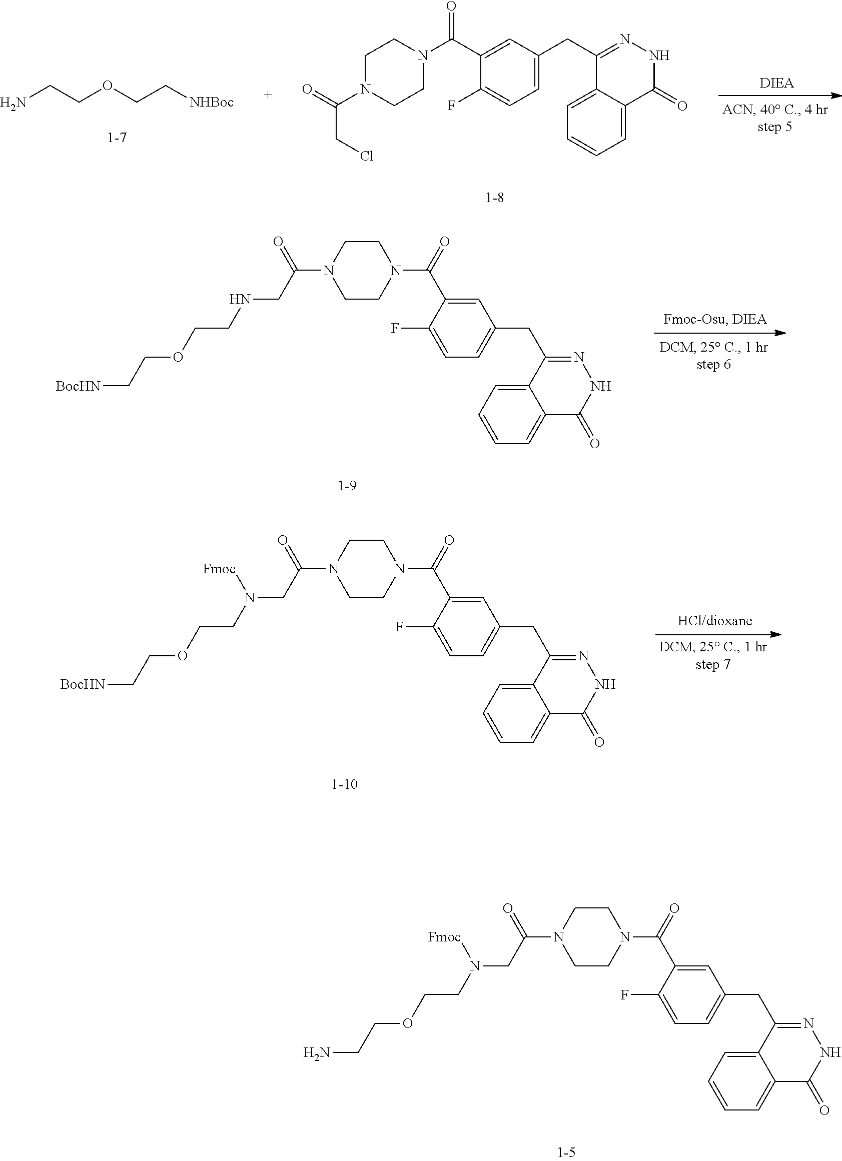

### Step 1: Synthesis of Intermediate 1-3

To a solution of intermediate 1-1 (200 mg, 704.15 gmol, 1 eq) in dioxane (2 mL) and H₂O (0.4 mL) was added Pd(dtbpf)Cl₂ (45.89 mg, 70.42 gmol, 0.1 eq), K₃PO₄ (448.41 mg, 2.11 mmol, 3 eq) and intermediate 1-2 (126.73 mg, 844.98 μmol, 1.2 eq). The mixture was stirred at 80° C. for 1 hr. The mixture was diluted with water (10 mL) and extracted with EA (10 mL*3). The combined organic layers dried over sodium sulfate, filtered and concentrated under reduced pressure to give a residue. The residue was purified by flash silica gel chromatography (ISCO; 20 g SepaFlash Silica Flash Column, Eluent of 0˜10% PE/EA gradient @40 mL/min), then the eluent was concentrated in vacuo to give intermediate 1-3 (200 mg, 627.26 μmol, 89.08% yield) as a brown oil.

### Step 2: Synthesis of Intermediate 1-4

To a solution of intermediate 1-3 (200 mg, 646.66 μmol, 1 eq) in MeOH (0.6 mL), H₂O (0.6 mL) and THF (0.6 mL) was added LiOH H₂O (81.41 mg, 1.94 mmol, 3 eq). The mixture was stirred at 25° C. for 0.5 hr. The pH of the mixture was acidified to 3-4 with 1N HCl, then diluted with water (10 mL) and extracted with EA (10 mL*3). The combined organic layers dried over sodium sulfate, filtered and concentrated under reduced pressure to give a residue. The residue was purified by flash silica gel chromatography (ISCO; 20 g SepaFlash Silica Flash Column, Eluent of 0˜70% PE/EA gradient @50 mL/min), then the eluent was concentrated in vacuo to give intermediate 1-4 (180 mg, 609.64 μmol, 94.28% yield) as a yellow solid. ¹H NMR (400 MHz, CHLOROFORM-d) δ=9.05 (s, 1H), 8.42 (s, 1H), 7.54-7.43 (m, 3H), 7.39 (m, 1H), 2.79 (m, 2H), 1.33 (m, 3H). ¹⁹F NMR (377 MHz, CHLOROFORM-d) δ=−60.57.

### Step 3: Synthesis of Intermediate 1-6

To a solution of intermediate 1-5 (80 mg, 104.00 μmol, 1 eq, HCl salt) in DMF (0.8 mL) was added HOAt (14.16 mg, 104.00 μmol, 14.55 μL, 1 eq), EDCI (59.81 mg, 311.99 μmol, 3 eq), NMM (52.59 mg, 519.98 μmol, 57.17 μL, 5 eq) and intermediate 1-4 (30.71 mg, 104.00 μmol, 1 eq). The mixture was stirred at 25° C. for 1 hr. The mixture was washed with water (2 mL) and extracted with EA (2 mL*3). The combined organic layers dried over sodium sulfate, filtered and concentrated under reduced pressure to give a residue. The residue was used for the next step without further purification. Intermediate 1-6 (120 mg, crude) was obtained as a yellow oil. LCMS (Method D): Rt=0.529 min, [M+H]⁺=1010.1.

### Step 4: Synthesis of I-486

To a solution of intermediate 1-6 (100 mg, 99.01 μmol, 1 eq) in THF (1 mL) was added piperidine (0.125 mL). The mixture was stirred at 25° C. for 0.2 hr. The reaction mixture was concentrated in vacuo to give a residue. The residue was purified by prep-HPLC (column: CD02-Waters Xbidge BEH C18 150*25*10 um; mobile phase: [water (NH3H2O)-ACN]; gradient: 35%-65% B over 10 min), and the eluent was concentrated under reduced pressure to remove ACN and lyophilized to give I-486 (37.31 mg, 47.21 μmol, 47.68% yield, 99.680% purity) as a white solid. LCMS (Method D): Rt=0.396 min, [M+H]⁺=788.4. ¹H NMR (400 MHz, METHANOL-d4) δ=9.04 (s, 1H), 8.43-8.33 (m, 2H), 7.98-7.90 (m, 1H), 7.89-7.80 (m, 2H), 7.59-7.52 (m, 2H), 7.50-7.41 (m, 2H), 7.37-7.30 (m, 2H), 7.14 (m, 1H), 4.37 (s, 2H), 3.74 (s, 1H), 3.71-3.58 (m, 9H), 3.54 (s, 2H), 3.48-3.43 (m, 2H), 3.37 (d, J=4.4 Hz, 1H), 3.22 (s, 1H), 2.80 (d, J=4.4 Hz, 2H), 2.77-2.69 (m, 2H), 1.31-1.24 (m, 3H). ¹⁹F NMR (377 MHz, METHANOL-d4) δ=−60.87, −120.70.

### Step 5: Synthesis of Intermediate 1-9

To a solution of intermediate 1-8 (7 g, 15.81 mmol, 1 eq) in ACN (70 mL) was added intermediate 1-7 (3.23 g, 15.81 mmol, 1 eq) and DIEA (6.13 g, 47.42 mmol, 8.26 mL, 3 eq). The mixture was stirred at 40° C. for 4 hr. The mixture was diluted with water (100 mL) and extracted with EA (50 mL*3). The combined organic layers were dried over sodium sulfate, filtered and concentrated under reduced pressure to give a residue. The residue was used for the next step without further purification. Intermediate 1-9 (9.6 g, crude) was obtained as a yellow oil. LCMS (Method D): Rt=0.292 min, [M+H]⁺=611.3.

### Step 6: Synthesis of Intermediate 1-10

To a solution oft intermediate 1-9 (10.6 g, 17.36 mmol, 1 eq) in DCM (110 mL) was added DIEA (4.49 g, 34.72 mmol, 6.05 mL, 2 eq) and FMOC-OSU (8.78 g, 26.04 mmol, 1.5 eq). The mixture was stirred at 25° C. for 1 hr. The mixture was diluted with water (100 mL) and extracted with DCM (50 mL*3). The combined organic layers were dried over sodium sulfate, filtered and concentrated under reduced pressure to give a residue. The residue was purified by prep-HPLC (column: Welch Ultimate XB—SiOH 250*70*10 um; mobile phase: [Hexane-EtOH]; gradient: 22%-52% B over 15 min), and the eluent was concentrated in vacuo to give intermediate 1-10 (6.5 g, 7.64 mmol, 44.00% yield, 97.876% purity) as a yellow oil. LCMS (Method D): Rt=0.481 min, [M+H]⁺=833.5.

### Step 7: Synthesis of Intermediate 1-5

To a solution of intermediate 1-10 (5.5 g, 6.60 mmol, 1 eq) in DCM (60 mL) was added HCl/dioxane (2 M, 60 mL). The mixture was stirred at 25° C. for 1 hr. The reaction mixture was concentrated in vacuo to give a product. The product was used for the next step without further purification. Intermediate 1-5 (5 g, 6.50 mmol, 98.43% yield, HCl salt) was obtained as a yellow solid. LCMS (Method D): Rt=0.352 min, [M+H]⁺=733.2. ¹H NMR (400 MHz, METHANOL-d4) δ=8.36 (d, J=7.6 Hz, 1H), 8.02-7.92 (m, 1H), 7.92-7.85 (m, 1H), 7.85-7.70 (m, 2H), 7.70-7.58 (m, 1H), 7.54 (s, 2H), 7.42-7.26 (m, 5H), 7.23-7.12 (m, 1H), 4.58 (d, J=2.8 Hz, 2H), 4.39 (d, J=11.2 Hz, 2H), 4.18 (s, 1H), 3.96-3.77 (m, 2H), 3.76-3.70 (m, 1H), 3.65-3.57 (m, 4H), 3.54 (s, 2H), 3.50-3.40 (m, 3H), 3.35 (m, 1H), 3.29-3.25 (m, 1H), 3.25-3.15 (m, 3H), 3.11-3.02 (m, 2H). ¹⁹F NMR (377 MHz, METHANOL-d4) δ=−110.59, −131.78.

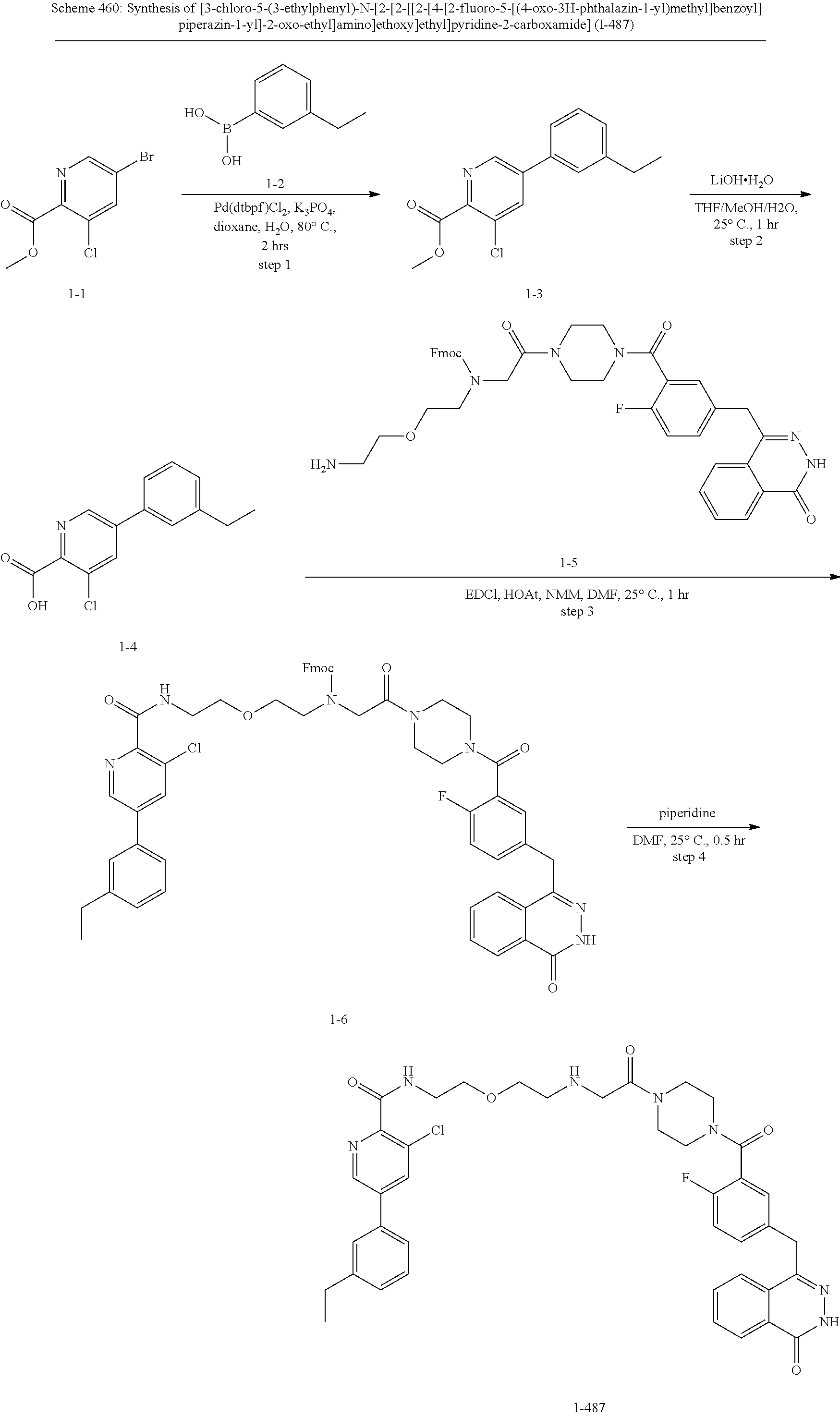

### Step 1: Synthesis of Intermediate 1-3.

To solution of Intermediate 1-1 (200 mg, 798.48 μmol, 1 eq) in dioxane (2 mL) and H₂O (0.4 mL) was added Intermediate 1-2 (119.76 mg, 798.48 μmol, 1 eq), Pd(dtbpf)Cl₂ (52.04 mg, 79.85 μmol, 0.1 eq) and K₃PO₄ (508.47 mg, 2.40 mmol, 3 eq) and the mixture was degassed and purged with N₂ 3 times, and then the mixture was stirred at 80° C. for 2 hr under N₂ atmosphere. The reaction mixture was quenched by addition of H₂O (5 mL), and then extracted with EA (5 mL*2), dried over Na₂SO₄, filtered and concentrated under reduced pressure to give a residue. The residue was purified by flash silica gel chromatography (ISCO; 20 g SepaFlash Silica Flash Column, Eluent of 0˜100% DCM/MeOH gradient @45 mL/min, PE/EA=1/1, Rf=0.7) and the eluent was concentrated under reduced pressure to give Intermediate 1-3 (140 mg, 482.85 μmol, 60.47% yield, 95.098% purity) as a yellow solid. LCMS (Method D): Retention time: 0.467 min, [M+H]⁺=276.0.

### Step 2: Synthesis of Intermediate 1-4.

To a solution of Intermediate 1-3 (50 mg, 181.34 μmol, 1 eq) in H₂O (0.2 mL), THF (0.2 mL) and MeOH (0.2 mL) was added LiOH·H₂O (22.83 mg, 544.01 μmol, 3 eq). The mixture was stirred at 25° C. for 1 hr. The reaction mixture was adjusted to pH 5 with 1M HCl and concentrated under reduced pressure to give a crude product. Intermediate 1-4 (45 mg, crude) was obtained as a yellow solid. LCMS (Method D): Retention time: 0.374 min, [M+H]⁺=262.0.

### Step 3: Synthesis of Intermediate 1-6.

To a solution of Intermediate 1-4 (45 mg, 171.95 μmol, 1 eq) in DMF (0.5 mL) was added Intermediate 1-5 (132.27 mg, 171.95 μmol, 1 eq, HCl salt), EDCI (98.89 mg, 515.85 μmol, 3 eq), HOAt (23.40 mg, 171.95 μmol, 24.05 μL, 1 eq) and NMM (86.96 mg, 859.75 μmol, 94.52 μL, 5 eq). The mixture was stirred at 25° C. for 1 hr. The mixture was concentrated under reduced pressure to give a crude product. The crude product was purified by reversed phase chromatography (0.1% FA) and the eluent was lyophilized to give the product. Intermediate 1-6 (70 mg, 71.05 μmol, 41.32% yield, 99.111% purity) was obtained as a white solid. LCMS (Method D): Retention time: 0.490 min, [M+H]⁺=976.2.

### Step 4: Synthesis of I-487

To a solution of Intermediate 1-6 (60 mg, 61.44 μmol, 1 eq) in DMF (0.6 mL) was added piperidine (10.46 mg, 122.89 μmol, 12.14 μL, 2 eq). The mixture was stirred at 25° C. for 0.5 hr. The mixture was concentrated under reduced pressure to give a crude product. The crude product was purified by reversed phase chromatography (0.1% FA) and the eluent was lyophilized to give the product. I-487 (25.45 mg, 31.45 μmol, 51.18% yield, 98.885% purity, FA salt) was obtained as a white solid. LCMS (Method D): Retention time: 0.387 min, [M+H]⁺=754.3. ¹H NMR (400 MHz, METHANOL-d4) δ=8.77 (d, J=7.2 Hz, 1H), 8.65-8.43 (m, 1H), 8.35 (d, J=7.6 Hz, 1H), 8.18 (d, J=9.6 Hz, 1H), 7.95-7.91 (m, 1H), 7.89-7.79 (m, 2H), 7.53 (s, 1H), 7.52-7.47 (m, 2H), 7.46-7.40 (m, 1H), 7.36-7.28 (m, 2H), 7.15-7.12 (m, 1H), 4.37 (s, 2H), 3.98 (s, 1H), 3.91 (s, 1H), 3.80-3.62 (m, 10H), 3.53-3.46 (m, 2H), 3.36 (s, 1H), 3.26 (s, 1H), 3.14 (d, J=3.6 Hz, 2H), 2.77-2.68 (m, 2H), 1.30-1.24 (m, 3H). ¹⁹F NMR (376 MHz, METHANOL-d₄) δ=−76.911, −120.657.

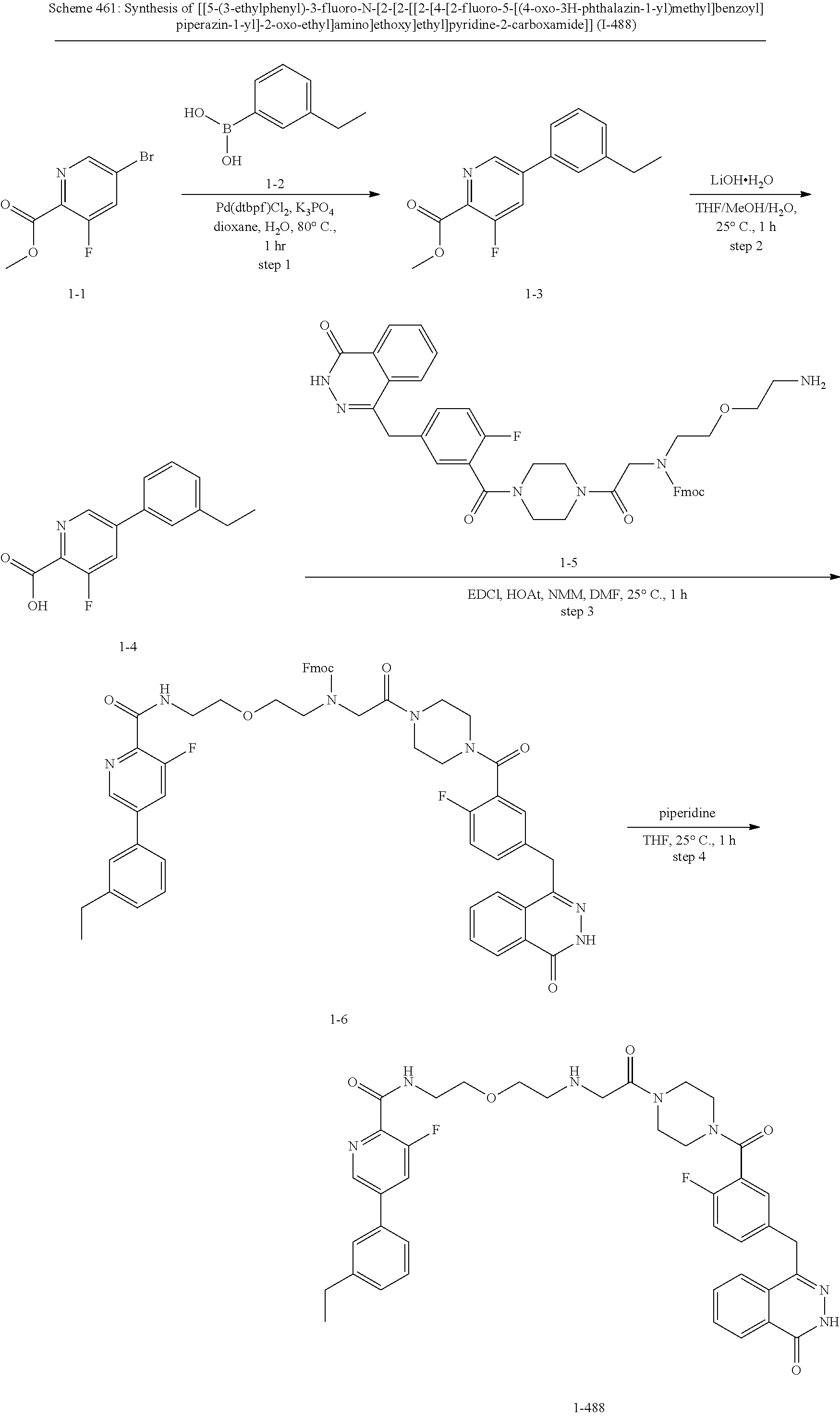

### Step 1: Synthesis of Intermediate 1-3.

To a solution of Intermediate 1-1 (300 mg, 1.28 mmol, 1 eq) in H₂O (1 mL) and dioxane (5 mL) was added Intermediate 1-2 (230.72 mg, 1.54 mmol, 1.2 eq), K₃PO₄ (816.33 mg, 3.85 mmol, 3 eq) and Pd(dtbpf)Cl₂ (83.55 mg, 128.19 μmol, 0.1 eq). The mixture was stirred at 80° C. for 1 hr under N₂ atmosphere. The reaction mixture was diluted with H₂O (10 mL) and extracted with EA (10 mL*2). The combined organic layers were dried over Na₂SO₄, filtered and concentrated under reduced pressure to give a residue. The residue was purified by flash silica gel chromatography (ISCO; 20 g SepaFlash Silica Flash Column, Eluent of 0-30% Ethyl acetate/Petroleum ether gradient @40 mL/min). The eluent was concentrated under reduced pressure to give Intermediate 1-3 (270 mg, 1.03 mmol, 80.58% yield, 99.2% purity) as a yellow oil. LCMS (Method D): Retention time: 0.429 min, [M+H]⁺=260.0. ¹H NMR (400 MHz, METHANOL-d₄) δ=8.78-8.74 (m, 1H), 8.08-7.96 (m, 1H), 7.61-7.51 (m, 2H), 7.47-7.41 (m, 1H), 7.38-7.30 (m, 1H), 3.99 (s, 3H), 2.80-2.67 (m, 2H), 1.34-1.23 (m, 3H). ¹⁹FNMR (376 MHz, MeOD-d₆) δ=−118.753.

### Step 2: Synthesis of Intermediate 1-4.

To a solution of Intermediate 1-3 (230 mg, 887.09 μmol, 1 eq) in MeOH (1 mL), THF (1 mL) and H₂O (1 mL) was added LiOH·H₂O (111.68 mg, 2.66 mmol, 3 eq). The mixture was stirred at 25° C. for 1 hr. The mixture was diluted with H₂O (5 mL) and HCl (1M) was added to adjust the pH to 4, and the mixture was extracted with EA (5 mL*2). The combined organic layers were dried over Na₂SO₄, filtered and concentrated under reduced pressure to give a residue. The residue was purified by reversed phase chromatography (neutral condition). The eluent was concentrated under reduced pressure to give Intermediate 1-4 (200 mg, 815.50 μmol, 91.93% yield, 100% purity) as a white solid. LCMS (Method D): Retention time: 0.396 min, [M+H]⁺=246.0. ¹H NMR (400 MHz, METHANOL-d₄) δ=8.80-8.73 (m, 1H), 8.03 (d, J=11.6 Hz, 1H), 7.66-7.51 (m, 2H), 7.49-7.41 (m, 1H), 7.35 (d, J=7.6 Hz, 1H), 2.83-2.67 (m, 2H), 1.34-1.25 (m, 3H). ¹⁹FNMR (377 MHz, MeOD-d6) δ=−118.753.

### Step 3: Synthesis of Intermediate 1-6.

To a solution of Intermediate 1-4 (31.88 mg, 130.00 μmol, 1 eq) in DMF (1 mL) was added Intermediate 1-5 (100 mg, 130.00 μmol, 1 eq, HCl salt), EDCI (74.76 mg, 389.99 μmol, 3 eq), HOAt (17.69 mg, 130.00 μmol, 18.18 μL, 1 eq) and NMM (65.74 mg, 649.98 μmol, 71.46 μL, 5 eq). The mixture was stirred at 25° C. for 1 hr. The reaction mixture was diluted with H₂O (5 mL) and extracted with EA (5 mL*2). The combined organic layers were washed with brine (10 mL*1), dried over Na₂SO₄, filtered and concentrated under reduced pressure to give a residue. The residue was purified by flash silica gel chromatography (ISCO; 12 g SepaFlash Silica Flash Column, Eluent of 0˜30% EA/PE @40 mL/min). The eluent was concentrated under reduced pressure to give Intermediate 1-5 (80 mg, 83.33 μmol, 64.10% yield, 100% purity) as a yellow oil. LCMS (Method D): Retention time: 0.522 min, [M+H]⁺=960.2.

### Step 4: Synthesis of I-488

To a solution of Intermediate 1-6 (65 mg, 67.71 μmol, 1 eq) in THF (80 L) was added piperidine (8.62 mg, 101.26 μmol, 10.00 μL, 1.50 eq). The mixture was stirred at 25° C. for 1 hr. The reaction mixture was diluted with H₂O (3 mL) and extracted with EA (3 mL*2). The combined organic layers were dried over Na₂SO₄, filtered and concentrated under reduced pressure to give a residue. The crude product was purified by prep-HPLC (column: CD04-Welch Utimate C18 150*25*7 um; mobile phase: [water (FA)-ACN]; gradient: 15%-45% B over 8 min). The eluent was concentrated under reduced pressure to remove ACN and then lyophilized to give I-488 (29.08 mg, 36.63 μmol, 54.10% yield, 98.721% purity, FA salt) as a white solid. LCMS (Method D): Retention time: 0.381 min, [M+H]⁺=738.3. ¹H NMR (400 MHz, METHANOL-d₄) δ=8.76-8.68 (m, 1H), 8.53 (s, 1H), 8.39-8.32 (m, 1H), 8.01-7.91 (m, 2H), 7.90-7.78 (m, 2H), 7.57-7.29 (m, 6H), 7.18-7.09 (m, 1H), 4.37 (s, 2H), 3.86-3.62 (m, 12H), 3.56-3.46 (m, 2H), 3.41-3.36 (m, 1H), 3.29-3.23 (m, 1H), 3.06-2.98 (m, 2H), 2.78-2.65 (m, 2H), 1.32-1.21 (m, 3H). ¹⁹F NMR (377 MHz, METHANOL-d₄) δ=−120.674, −122.573.

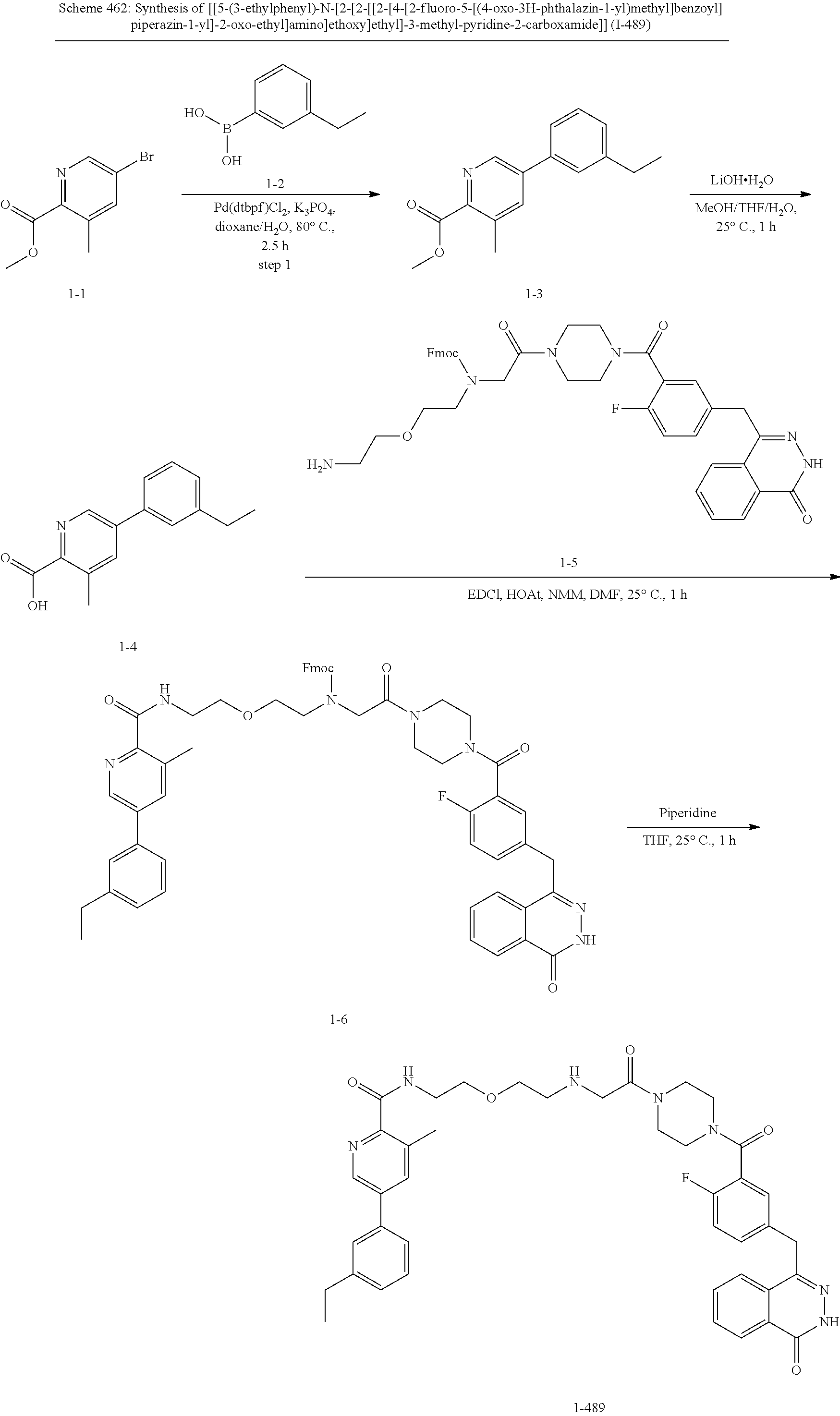

### Step 1: Synthesis of Intermediate 1-3

A mixture of Intermediate 1-1 (250 mg, 1.09 mmol, 1 eq), Intermediate 1-2 (244.47 mg, 1.63 mmol, 1.5 eq), K₃PO₄ (692.00 mg, 3.26 mmol, 3 eq) and Pd(dtbpf)Cl₂ (70.82 mg, 108.67 μmol, 0.1 eq) in dioxane (2 mL) and H₂O (0.4 mL) was degassed and purged with N₂ 3 times, and then the mixture was stirred at 80° C. for 2.5 hr under N₂ atmosphere. The residue was diluted with H₂O (5 mL) and extracted with EA (5 mL*3), dried over Na₂SO₄, filtered and concentrated under reduced pressure to give a residue. The residue was purified by flash silica gel chromatography (ISCO; 12 g SepaFlash Silica Flash Column, Eluent of 0-100% Petroleum ether/Ethyl acetate@40 mL/min). The eluent was concentrated under reduced pressure to give Intermediate 1-3 (320 mg, crude) as a yellow oil. LCMS (Method D): Retention time: 0.432 min, [M+H]⁺=256.0.

### Step 2: Synthesis of Intermediate 1-4

To a solution of Intermediate 1-3 (290 mg, 1.14 mmol, 1 eq) in MeOH (1 mL), THF (1 mL) and H₂O (1 mL) was added LiOH·H₂O (95.33 mg, 2.27 mmol, 2 eq). The mixture was stirred at 25° C. for 1 hr. The reaction mixture was adjusted to pH=5 with 1M HCl, and extracted with EA (5 mL*3), dried over Na₂SO₄, filtered and concentrated under reduced pressure to give Intermediate 1-4 (250 mg, 1.04 mmol, 91.22% yield, 100.0000% purity) as a brown solid. LCMS (Method D): Retention time: 0.299 min, [M+H]⁺=242.1

### Step 3: Synthesis of Intermediate 1-6

To a solution of Intermediate 1-4 (25 mg, 103.61 μmol, 1 eq) in DMF (1 mL) was added HOAt (14.10 mg, 103.61 μmol, 14.49 μL, 1 eq), EDCI (59.59 mg, 310.84 μmol, 3 eq), NMM (52.40 mg, 518.06 μmol, 56.96 μL, 5 eq) and Intermediate 1-5 (79.70 mg, 103.61 μmol, 1 eq, HCl salt). The mixture was stirred at 25° C. for 1 hr. The residue was diluted with H₂O (5 mL) and extracted with EA (5 mL*3), dried over Na₂SO₄, filtered and concentrated under reduced pressure to give a residue. The residue was purified by flash silica gel chromatography (ISCO; 20 g SepaFlash Silica Flash Column, Eluent of 0˜100% Dichloromethane/Methanol @60 mL/min). The eluent was concentrated under reduced pressure to give Intermediate 1-6 (120 mg, crude) as a yellow solid. LCMS (Method D): Retention time: 0.499 min, [M+H]⁺=956.3.

### Step 4: Synthesis of I-489

To a solution of Intermediate 1-6 (100 mg, 104.60 μmol, 1 eq) in THF (1 mL) was added piperidine (107.78 mg, 1.27 mmol, 0.125 mL, 12.10 eq). The mixture was stirred at 25° C. for 1 hr. The residue was diluted with H₂O (5 mL) and extracted with EA (5 mL*3), dried over Na₂SO₄, filtered and concentrated under reduced pressure to give a residue. The crude product was purified by reversed phase chromatography (0.1% FA), the eluent was concentrated to remove MeCN and then lyophilized to give I-489 (42.23 mg, 53.77 μmol, 51.41% yield, 99.297% purity, FA salt) as a white solid. LCMS (Method D): Retention time: 0.395 min, [M+H]⁺=734.3. ¹H NMR (400 MHz, METHANOL-d4) δ=8.66 (d, J=8.4 Hz, 1H), 8.47 (s, 1H), 8.36 (d, J=8.0 Hz, 1H), 7.94 (d, J=8.4 Hz, 2H), 7.88-7.79 (m, 2H), 7.53-7.45 (m, 3H), 7.43-7.37 (m, 1H), 7.36-7.25 (m, 2H), 7.17-7.11 (m, 1H), 4.36 (d, J=0.8 Hz, 2H), 4.08-3.94 (m, 2H), 3.78 (d, J=4.4 Hz, 2H), 3.75-3.67 (m, 4H), 3.65 (s, 3H), 3.53-3.44 (m, 2H), 3.29-3.12 (m, 4H), 2.81-2.59 (m, 6H), 1.30-1.23 (m, 3H). ¹⁹F NMR (377 MHz, METHANOL-d4) δ=−120.627.

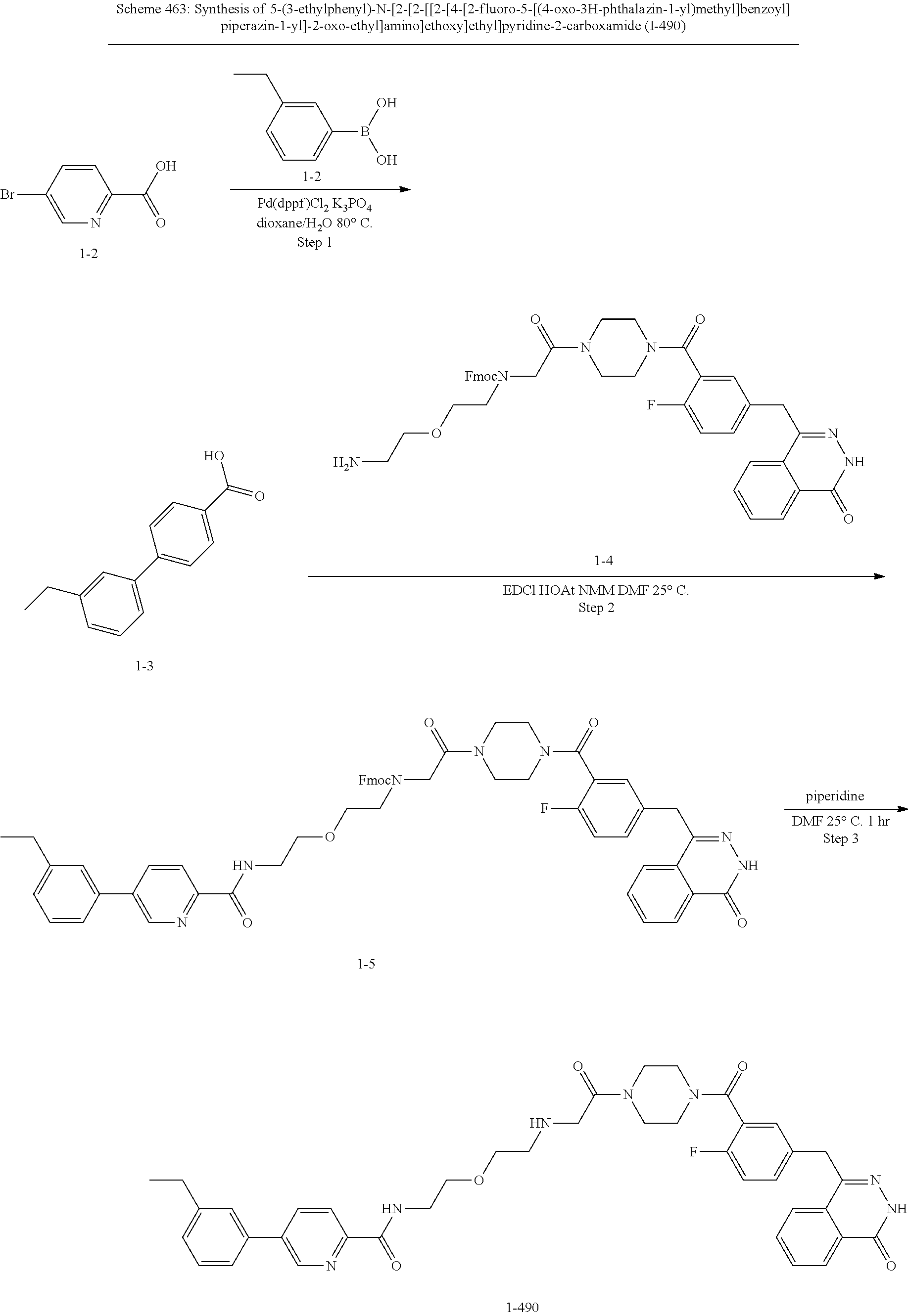

### Step 1: Synthesis of Intermediate 1-3

A mixture of Intermediate 1-1 (200 mg, 990.07 μmol, 1 eq), Intermediate 1-2 (193.04 mg, 1.29 mmol, 1.3 eq), K₃PO₄ (630.48 mg, 2.97 mmol, 3 eq) and Pd(dppf)Cl₂ (144.89 mg, 198.01 μmol, 0.2 eq) in dioxane (2 mL) and H₂O (0.5 mL) was degassed and purged with N₂ 3 times, and then the mixture was stirred at 80° C. for 1 hr under N₂ atmosphere. The reaction mixture was quenched by addition of water (5 mL) at 25° C., and then extracted with EA (5 mL*3). The pH of the aqueous phase was adjusted to 5-6, and then the aqueous phase was extracted with EA (5 mL*3). The organic layers were dried over Na₂SO₄, filtered and concentrated under reduced pressure to give a residue. The crude product was used in the next step without further purification. Intermediate 1-3 (180 mg, crude) was obtained as a yellow solid. LCMS (Method E): Rt=0.452 min, [M+H]⁺=228.1.

### Step 2: Synthesis of Intermediate 1-5

To a solution of Intermediate 1-3 (80 mg, 352.02 μmol, 1 eq) and Intermediate 1-4 (257.96 mg, 352.02 μmol, 1 eq) in DMF (1 mL) was added EDCI (202.45 mg, 1.06 mmol, 3 eq), NMM (178.03 mg, 1.76 mmol, 193.51 μL, 5 eq) and HOAt (47.91 mg, 352.02 μmol, 49.24 μL, 1 eq). The mixture was stirred at 25° C. for 1 hr. The reaction mixture was quenched by addition of water (5 mL) at 25° C., and then the mixture was extracted with EA (5 mL*3). Then the organic layers were dried over Na₂SO₄, filtered and concentrated under reduced pressure to give a residue. The crude product was triturated with PE:EA=3:1 (5 mL) at 25° C. for 30 min to give Intermediate 1-5 (120 mg, crude) as a yellow solid. LCMS (Method E): Rt=0.618 min, [M+H]⁺=942.4.

### Step 3: Synthesis of I-490

To a solution of Intermediate 1-5 (50 mg, 53.08 μmol, 1 eq) in DMF (0.5 mL) was added piperidine (431.10 mg, 5.06 mmol, 0.5 mL, 95.39 eq). The mixture was stirred at 25° C. for 1 hr. The reaction mixture was filtered to give a residue. The residue was purified by prep-HPLC (column: Waters Xbridge 150*25 mm*5 um; mobile phase: [water (ammonia hydroxide v/v)-ACN]; gradient: 23%-53% B over 10 min). I-490 (23 mg, 31.95 μmol, 60.20% yield, 100% purity) was obtained as a white solid. LCMS (Method E): Rt=0.480 min, [M+H]⁺=720.4. ¹H NMR (400 MHz, METHANOL-d₄) δ=8.85 (d, J=8.0 Hz, 1H), 8.40-8.32 (m, 1H), 8.21-8.07 (m, 2H), 7.97-7.91 (m, 1H), 7.84 (m, 2H), 7.57-7.44 (m, 3H), 7.44-7.24 (m, 3H), 7.19-7.07 (m, 1H), 4.36 (s, 2H), 3.84-3.56 (m, 10H), 3.55-3.44 (m, 4H), 3.36 (d, J=4.0 Hz, 1H), 3.24 (d, J=2.8 Hz, 1H), 2.81 (d, J=4.4 Hz, 2H), 2.77-2.64 (m, 2H), 1.27 (m, 3H). ¹⁹F NMR (377 MHz, METHANOL-d₄) δ=−120.671.

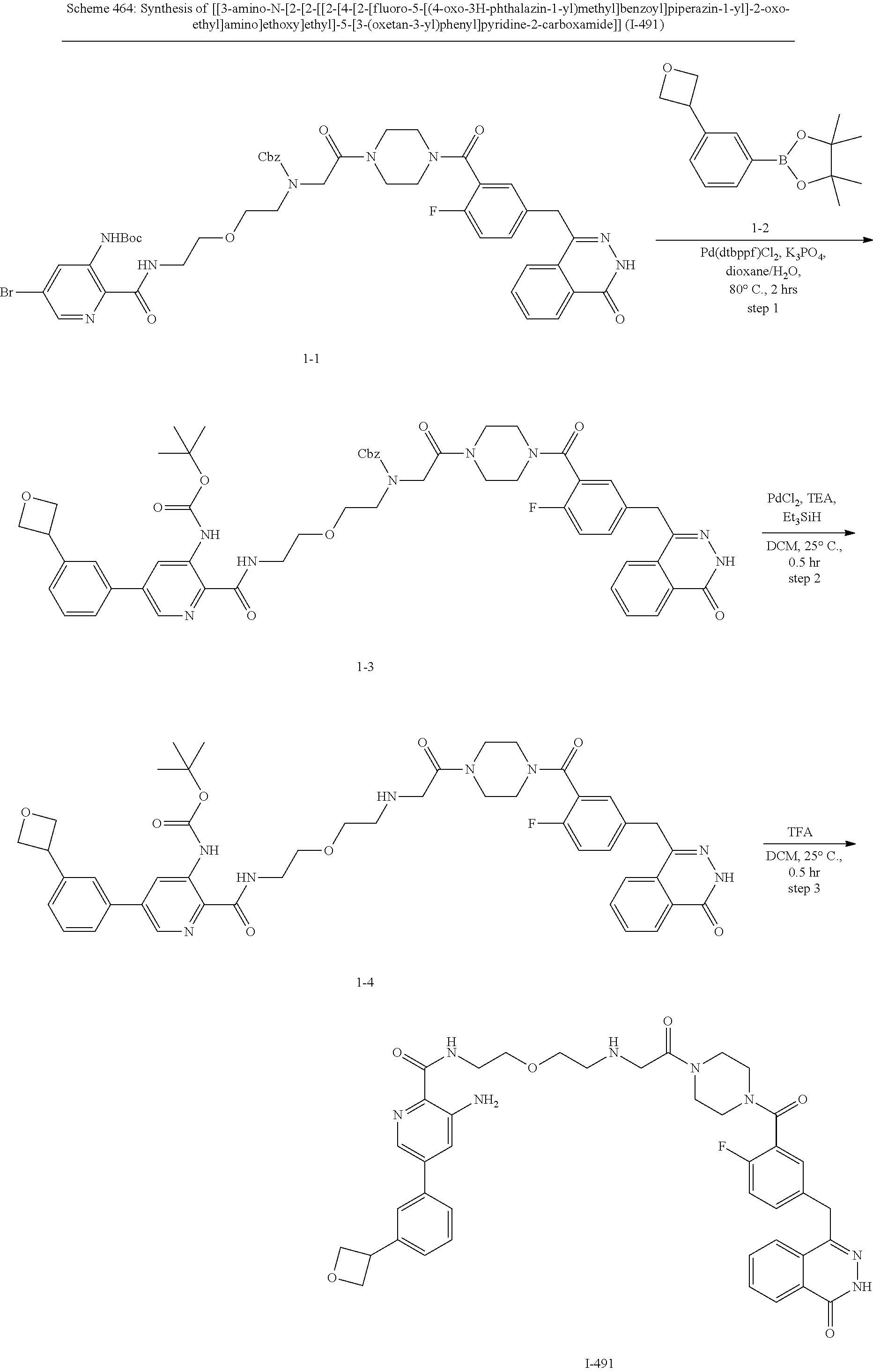 [SMILES: CC(C)(C)OC(=O)Nc1cc(Br)cnc1C(=O)NCCOCCN(CC(=O)N1CCN(C(=O)c2cc(Cc3n[nH]c(=O)c4ccccc34)ccc2F)CC1)C(=O)OCc1ccccc1 ; CC1(C)OB(c2cccc(C3COC3)c2)OC1(C)C ; CC(C)(C)OC(=O)Nc1cc(-c2cccc(C3COC3)c2)cnc1C(=O)NCCOCCN(CC(=O)N1CCN(C(=O)c2cc(Cc3n[nH]c(=O)c4ccccc34)ccc2F)CC1)C(=O)OCc1ccccc1 ; CC(C)(C)OC(=O)Nc1cc(-c2cccc(C3COC3)c2)cnc1C(=O)NCCOCCNCC(=O)N1CCN(C(=O)c2cc(Cc3n[nH]c(=O)c4ccccc34)ccc2F)CC1 ; Nc1cc(-c2cccc(C3COC3)c2)cnc1C(=O)NCCOCCNCC(=O)N1CCN(C(=O)c2cc(Cc3n[nH]c(=O)c4ccccc34)ccc2F)CC1]

### Step 1: Synthesis of Intermediate 1-3

A mixture of Intermediate 1-1 (280 mg, 296.67 μmol, 1 eq), Intermediate 1-2 (77.17 mg, 296.67 μmol, 1 eq), Pd(dtbpf)Cl₂ (19.34 mg, 29.67 μmol, 0.1 eq), K₃PO₄ (188.92 mg, 890.01 μmol, 3 eq) in dioxane (2.5 mL) and H₂O (0.5 mL) was degassed and purged with N₂ 3 times, and then the mixture was stirred at 80° C. for 2 hr under N₂ atmosphere. LCMS showed Intermediate 1-1 was consumed completely and 82% of desired mass. The reaction mixture was quenched by addition H₂O (5 mL) at 25° C., and extracted with DCM 15 mL (5 mL*3), dried over Na₂SO₄, filtered and concentrated under reduced pressure to give a crude product. The crude product was purified by reversed phase chromatography (neutral condition) and lyophilized to give Intermediate 1-3 (150 mg, 150.44 μmol, 50.71% yield, 100% purity) as a white solid. LCMS: Retention time: 0.575 min, [M+H]⁺=997.4.

### Step 2: Synthesis of Intermediate 1-4

To a solution of Intermediate 1-3 (50 mg, 50.15 μmol, 1 eq) in DCM (0.5 mL) was added PdCl₂ (4.45 mg, 25.07 μmol, 0.5 eq) and TEA (15.22 mg, 150.44 μmol, 20.94 μL, 3 eq). The reaction mixture was degassed with N₂. Then Et₃SiH (69.97 mg, 601.76 μmol, 96.11 μL, 12 eq) was added. The mixture was stirred at 25° C. for 1 hr. The reaction mixture was adjusted to pH to 7-8 by NaHCO₃, diluted with H₂O (5 mL), extracted with DCM (5 mL*3), dried over Na₂SO₄, filtered and concentrated under reduced pressure to give a crude product. The crude product was used in the next step. Intermediate 1-4 (42.37 mg, crude) was obtained as a white solid. LCMS (Method D): Retention time: 0.394 min, [M+H]⁺=863.3.

### Step 3: Synthesis of I-491

To a solution of Intermediate 1-4 (20 mg, 23.18 μmol, 1 eq) in DCM (0.5 mL) was added TFA (153.50 mg, 1.35 mmol, 0.1 mL). The mixture was stirred at 25° C. for 0.5 hr. The reaction mixture was adjusted the pH to 7-8 by NaHCO₃, diluted with H₂O (5 mL), extracted with DCM (5 mL*3), dried over Na₂SO₄, filtered and concentrated under reduced pressure to give the crude product. The crude product was purified by prep-HPLC (column: CD01-Phenomenex luna C18 150*25*10 um; mobile phase: [water (FA)-ACN]; gradient: 12%-42% B over 10 min) and lyophilized to give the product. I-491 (9.24 mg, 11.28 μmol, 48.66% yield, 98.729% purity, FA salt) was obtained as a white solid. LCMS (Method D): Retention time: 0.324 min, [M+H]⁺=763.3. ¹H NMR (400 MHz, METHANOL-d₄) δ=8.47 (s, 1H), 8.39-8.34 (m, 1H), 8.09 (d, J=18.4 Hz, 1H), 7.96-7.91 (m, 1H), 7.89-7.80 (m, 2H), 7.68-7.61 (m, 1H), 7.49 (d, J=9.6 Hz, 4H), 7.37 (d, J=11.2 Hz, 1H), 7.31 (s, 1H), 7.17-7.09 (m, 1H), 5.17-5.06 (m, 2H), 4.79-4.74 (m, 2H), 4.38-4.29 (m, 3H), 4.08-3.96 (m, 2H), 3.84-3.58 (m, 10H), 3.51-3.44 (m, 2H), 3.22 (d, J=4.6 Hz, 4H). ¹⁹F NMR (377 MHz, METHANOL-d₄) δ=−120.597.

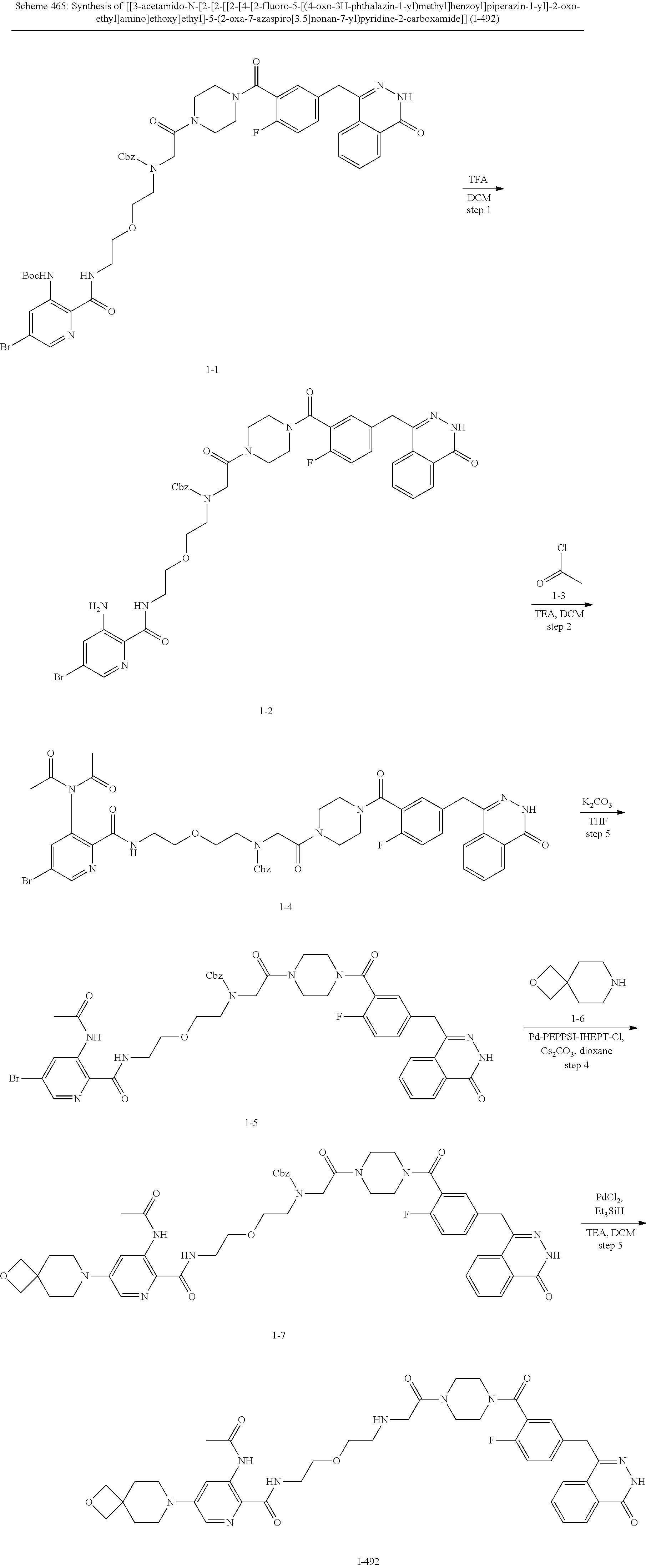

### Step 1: Synthesis of Intermediate 1-2

To a solution of Intermediate 1-1 (300 mg, 317.86 μmol, 1 eq) in DCM (1 mL) was added TFA (3 mL), then the mixture was stirred at 25° C. for 1 hour. The reaction mixture was added to a NaHCO₃ solution (about 5 mL) to adjust the pH to 7-8, then it was extracted with DCM 15 mL (5 mL*3). The combined organic layers were concentrated under reduced pressure to give a residue. Intermediate 1-3 (240 mg, 284.46 μmol, 89.49% yield) was obtained as a yellow solid. LCMS (Method D): Retention time: 0.445 min.

### Step 2: Synthesis of Intermediate 1-4

To a mixture of Intermediate 1-2 (240 mg, 284.46 μmol, 1 eq) in DCM (3 mL) was added TEA (86.35 mg, 853.39 μmol, 118.78 μL, 3 eq) and Intermediate 1-3 (17.86 mg, 227.57 μmol, 16.18 μL, 0.8 eq) at 0° C., then the mixture was stirred at 0° C. for 1 hour. The reaction mixture was diluted with water (3 mL) and extracted with DCM 10 mL (5 mL*2). The combined organic layers were concentrated under reduced pressure to give a residue. Intermediate 1-4 (240 mg, 258.69 μmol, 90.94% yield) was obtained as a yellow solid. LCMS (Method D): Retention time: 0.484 min, [M+H]⁺=927.2.

### Step 3: Synthesis of Intermediate 1-5

To a mixture of Intermediate 1-4 (150 mg, 506.14 μmol, 1 eq) in THF (3 mL) was added K₂CO₃ (178.76 mg, 1.29 mmol, 5 eq), then the mixture was stirred at 80° C. for 1 hour. The reaction mixture was diluted with water (10 mL) and extracted with DCM (10 mL*2). The crude product was purified by reversed phase chromatography (0.1% FA condition). The eluent was concentrated under reduced pressure to remove ACN and then lyophilized to give the product. Intermediate 1-5 (145 mg, 163.00 μmol, 63.01% yield, 99.566% purity) was obtained as a white solid. LCMS (Method D): Retention time: 0.461 min, [M+H]⁺=885.0.

### Step 4: Synthesis of Intermediate 1-7

To a mixture of Intermediate 1-5 (80 mg, 90.32 μmol, 1 eq) and Intermediate 1-6 (34.46 mg, 270.96 μmol, 3 eq) in dioxane (1 mL) was added Cs₂CO₃ (88.28 mg, 270.96 μmol, 3 eq) and Pd-PEPPSI-IHEPT-Cl (8.79 mg, 9.03 μmol, 0.1 eq). Then the mixture was stirred at 90° C. for 1 hour under N₂. The reaction mixture was diluted with water (10 mL) and extracted with EA (5 mL*2). The combined organic layers were concentrated under reduced pressure to give a residue. Intermediate 1-6 (60 mg, 64.38 μmol, 71.28% yield) was obtained as a yellow solid. LCMS (Method D): Retention time: 0.446 min, [M+H]⁺=932.3.

### Step 5: Synthesis of I-492

To a solution of Intermediate 1-7 (60 mg, 64.38 μmol, 1 eq) in DCM (1 mL) was added PdCl₂ (2.28 mg, 12.88 μmol, 0.2 eq) and TEA (13.03 mg, 128.75 μmol, 17.92 μL, 2 eq). The reaction mixture was degassed with N₂. Then a mixture of Et₃SiH (29.94 mg, 257.51 μmol, 41.13 μL, 4 eq) in DCM (0.5 mL) was added dropwise at 10° C. Then the mixture was stirred at 25° C. for 2 hours. The reaction mixture was concentrated under reduced pressure to give a residue. The crude product was purified by reversed phase chromatography (0.1% FA condition). The eluent was concentrated under reduced pressure to remove ACN and then lyophilized to give the product. I-492 (9.71 mg, 11.56 μmol, 17.96% yield, 95% purity) was obtained as a yellow solid. LCMS (Method D): Retention time: 0.336 min, [M+H]⁺=798.3. ¹H NMR (400 MHz, METHANOL-d₄) δ=8.48 (s, 1H), 8.39-8.31 (m, 1H), 8.03-7.91 (m, 2H), 7.90-7.80 (m, 2H), 7.53-7.46 (m, 1H), 7.35 (d, J=4.0 Hz, 1H), 7.17-7.16 (m, 1H), 4.47 (d, J=7.6 Hz, 4H), 4.38 (s, 2H), 4.03-3.91 (m, 2H), 3.82-3.53 (m, 10H), 3.51-3.47 (m, 1H), 3.43 (s, 1H), 3.33 (s, 2H), 3.30-3.25 (m, 4H), 3.22-3.17 (m, 2H), 2.19 (d, J=2.4 Hz, 3H), 1.98-1.92 (m, 4H). ¹⁹F NMR (377 MHz, METHANOL-d₄) δ=−120.517.

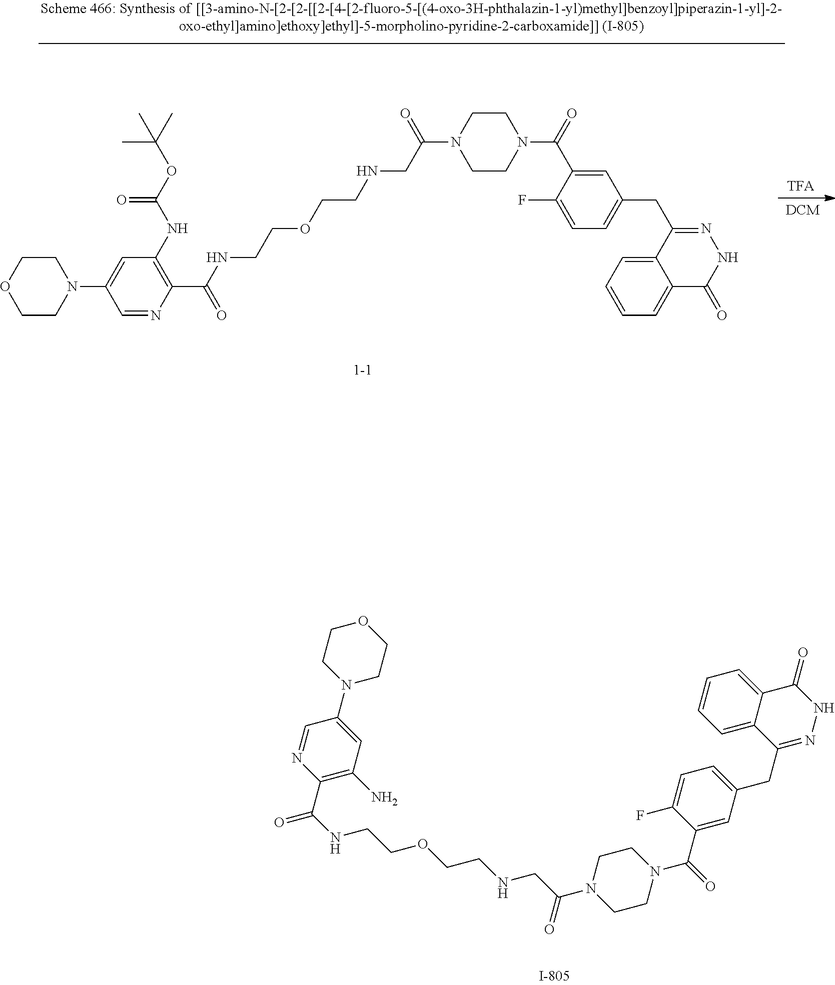 [SMILES: CC(C)(C)OC(=O)Nc1cc(N2CCOCC2)cnc1C(=O)NCCOCCNCC(=O)N1CCN(C(=O)c2cc(Cc3n[nH]c(=O)c4ccccc34)ccc2F)CC1 ; Nc1cc(N2CCOCC2)cnc1C(=O)NCCOCCNCC(=O)N1CCN(C(=O)c2cc(Cc3n[nH]c(=O)c4ccccc34)ccc2F)CC1]

To a mixture of Intermediate 1-1 (20 mg, 24.51 μmol, 1 eq) in DCM (0.2 mL) was added TFA (307.00 mg, 2.69 mmol, 0.2 mL, 109.84 eq). The mixture was stirred at 25° C. for 1 hr. Then the reaction mixture was concentrated and purified by reversed phase chromatography (neutral condition) and the eluent was lyophilized to give product. I-805 (5 mg, 6.03 μmol, 24.58% yield, 100% purity, TFA salt) was obtained as a white solid. LCMS (Method H): Retention time: 0.506 min, [M+H]⁺=716.4. ¹H NMR (400 MHz, METHANOL-d₄) δ=8.41-8.33 (m, 1H), 7.96-7.92 (m, 1H), 7.90-7.81 (m, 2H), 7.68-7.59 (m, 1H), 7.55-7.46 (m, 1H), 7.38-7.32 (m, 1H), 7.17 (t, J=9.0 Hz, 1H), 6.47 (d, J=8.4 Hz, 1H), 4.39 (s, 2H), 3.87 (s, 1H), 3.80 (d, J=4.4 Hz, 5H), 3.73 (s, 4H), 3.66 (s, 3H), 3.60-3.40 (m, 5H), 3.21 (d, J=4.4 Hz, 6H), 3.10-3.04 (m, 2H). ¹⁹F NMR (377 MHz, METHANOL-d₄) δ=−76.949, −120.657.

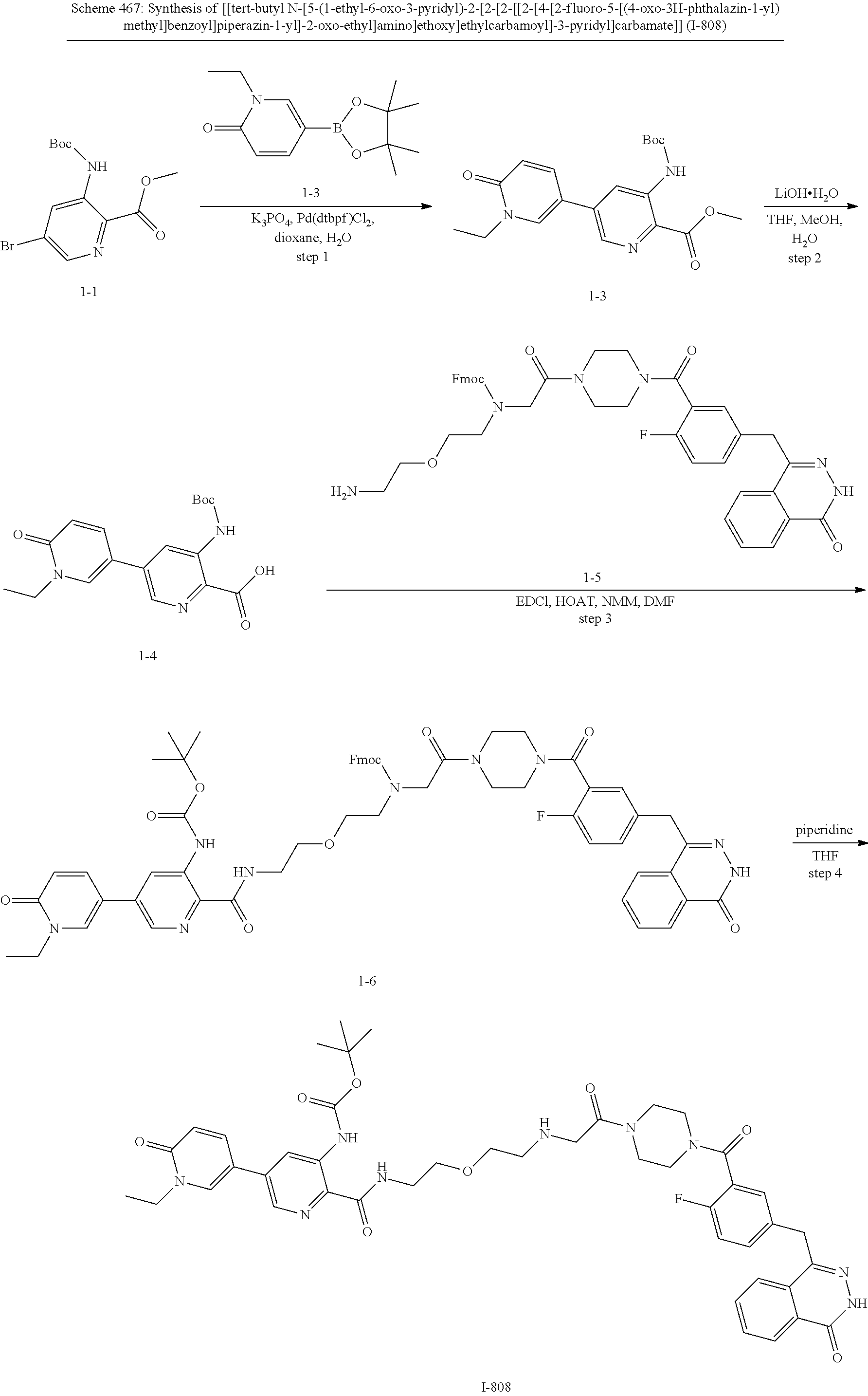

### Step 1: Synthesis of Intermediate 1-3

A mixture of Intermediate 1-1 (2 g, 6.04 mmol, 1 eq), Intermediate 1-2 (1.81 g, 7.25 mmol, 1.2 eq), K₃PO₄ (3.85 g, 18.12 mmol, 3 eq), and Pd(dtbpf)Cl₂ (393.61 mg, 603.93 μmol, 0.1 eq) in dioxane (15 mL) and H₂O (3 mL) was degassed and purged with N₂ 3 times, and then the mixture was stirred at 80° C. for 1 hr under N₂ atmosphere. The reaction mixture was diluted with water (20 mL) and extracted with EA (20 mL*3). The combined organic layers were dried over Na₂SO₄, filtered and concentrated under reduced pressure to give a residue. The residue was purified by silica gel column chromatography (ISCO; 80 g SepaFlash Silica Flash Column, Eluent of 0˜100% PE/EA gradient @60 mL/min). The eluent was concentrated to afford the product. Intermediate 1-3 (1.92 g, 5.14 mmol, 85.14% yield) was obtained as a yellow solid. LCMS (Method D): Retention time: 0.38 min, [M+H]⁺=374.1.

### Step 2: Synthesis of Intermediate 1-4

To a solution of Intermediate 1-3 (1.8 g, 4.82 mmol, 1 eq) in THF (6 mL), MeOH (6 mL) and H₂O (6 mL) was added LiOH H₂O (606.86 mg, 14.46 mmol, 3 eq). The mixture was stirred at 25° C. for 2 hrs. The mixture was adjusted to pH=5 with citric acid and extracted with EA (30 mL*3). The combined organic layers were dried over Na₂SO₄, filtered and concentrated under reduced pressure to give a residue. The residue was used for the next step without further purification. Intermediate 1-4 (1.8 g, crude) was obtained as a yellow oil. LCMS (Method D): Retention time: 0.339 min, [M+H]⁺=360.0.

### Step 3: Synthesis of Intermediate 1-6

To a solution of Intermediate 1-4 (100 mg, 278.26 μmol, 2 eq) in DMF (1 mL) was added HOAt (37.87 mg, 278.26 μmol, 38.93 μL, 2 eq) and EDCI (133.36 mg, 695.65 μmol, 5 eq), NMM (140.73 mg, 1.39 mmol, 152.96 μL, 10 eq), and then Intermediate 1-5 (107.03 mg, 139.13 μmol, 1 eq, HCl salt) was added. The mixture was stirred at 25° C. for 1 hr. The reaction mixture was diluted with water (10 mL) and extracted with EA (5 mL*3). The combined organic layers were dried over Na₂SO₄, filtered and concentrated under reduced pressure to give a residue. The residue was purified by reversed phase chromatography (0.1% FA), and the eluent was lyophilized to give a product. Intermediate 1-6 (105 mg, 97.75 μmol, 70.26% yield) was obtained as a gray solid. LCMS (Method D): Retention time: 0.525 min, [M+H]⁺=1074.7.

### Step 4: Synthesis of I-808

To a solution of Intermediate 1-6 (100 mg, 93.10 μmol, 1 eq) in THF (0.8 mL) was added piperidine (86.22 mg, 1.01 mmol, 0.1 mL, 10.88 eq). The mixture was stirred at 25° C. for 1 hr. The reaction mixture was concentrated under reduce pressure to give a residue. The residue was purified by reversed phase chromatography (0.1% FA), then the eluent was lyophilized to give product. I-808 (20 mg, 21.91 μmol, 23.54% yield, 98.377% purity, FA salt) was obtained as a white solid. LCMS (Method D): Retention time: 0.362 min, [M+H]⁺=852.4. ¹H NMR (377 MHz, METHANOL-d₄) δ=8.90 (s, 1H), 8.52 (s, 1H), 8.43 (d, J=6.4 Hz, 1H), 8.36 (d, J=8.0 Hz, 1H), 8.16 (d, J=2.4 Hz, 1H), 7.96-7.88 (m, 2H), 7.88-7.79 (m, 2H), 7.48 (d, J=2.8 Hz, 1H), 7.36-7.31 (m, 1H), 7.15 (t, J=8.8 Hz, 1H), 6.67 (t, J=9.6 Hz, 1H), 4.37 (s, 2H), 4.17-4.09 (m, 2H), 3.76-3.59 (m, 11H), 3.49 (d, J=4.4 Hz, 2H), 3.35 (d, J=3.6 Hz, 1H), 3.27 (s, 1H), 3.02-2.92 (m, 2H), 1.54 (s, 9H), 1.42-1.35 (m, 3H). ¹⁹F NMR (377 MHz, METHANOL-d₄) δ=−76.939, −120.652.

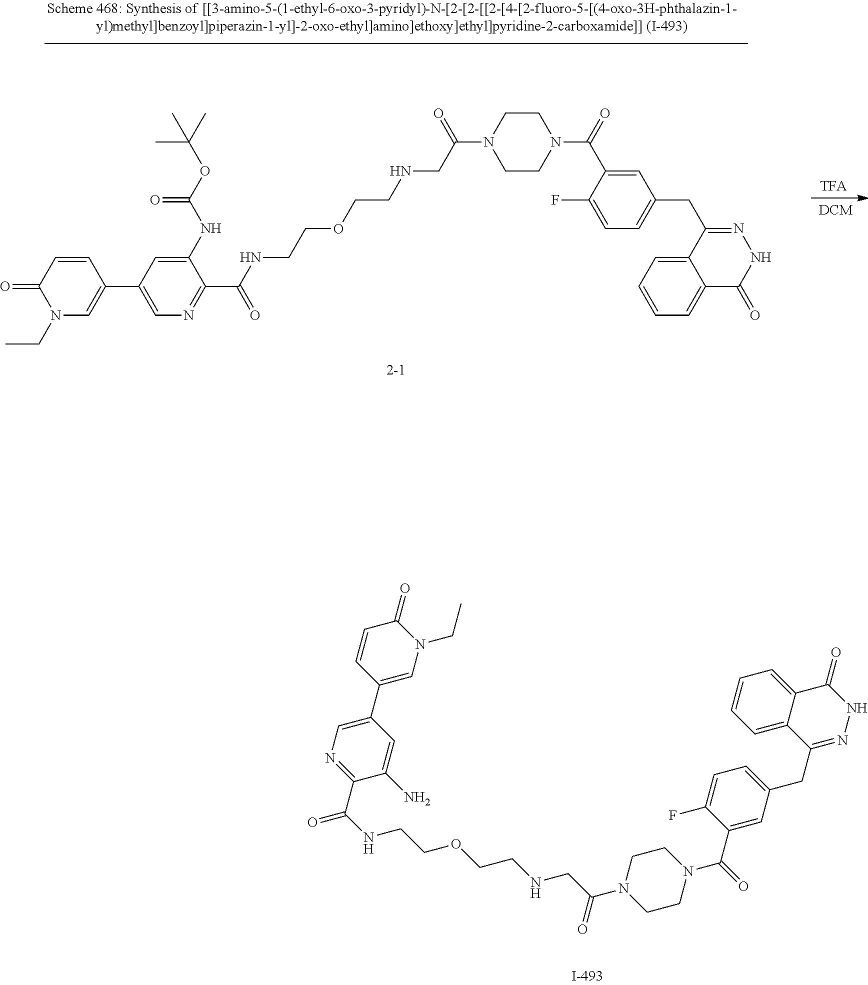 [SMILES: CCn1cc(-c2cnc(C(=O)NCCOCCNCC(=O)N3CCN(C(=O)c4cc(Cc5n[nH]c(=O)c6ccccc56)ccc4F)CC3)c(NC(=O)OC(C)(C)C)c2)ccc1=O ; CCn1cc(-c2cnc(C(=O)NCCOCCNCC(=O)N3CCN(C(=O)c4cc(Cc5n[nH]c(=O)c6ccccc56)ccc4F)CC3)c(N)c2)ccc1=O]

To a solution of Intermediate 2-1 (60 mg, 70.43 μmol, 1 eq) in DCM (0.3 mL) was added TFA (460.50 mg, 4.04 mmol, 0.3 mL). The mixture was stirred at 25° C. for 12 hr. The reaction mixture was concentrated under reduced pressure to give a residue. The residue was purified by reversed phase chromatography (0.10% FA salt), then the eluent was lyophilized to give product. I-493 (14 mg, 16.17 mol, 22.96% yield, TFA salt) was obtained as a white solid. LCMS (Method D): Retention time: 0.320 min, [M+H]⁺=752.4. ¹H NMR (400 MHz, METHANOL-d₄) δ=8.37 (d, J=7.6 Hz, 1H), 8.08-7.99 (m, 2H), 7.96-7.92 (m, 1H), 7.90-7.80 (m, 3H), 7.53-7.47 (m, 1H), 7.37-7.30 (m, 1H), 7.29-7.23 (m, 1H), 7.16 (t, J=9.2 Hz, 1H), 6.69-6.60 (m, 1H), 4.38 (s, 2H), 4.18-4.05 (m, 4H), 3.85-3.57 (m, 10H), 3.51 (t, J=4.8 Hz, 1H), 3.43 (s, 1H), 3.29 (s, 4H), 1.40-1.30 (m, 3H). ¹⁹F NMR (377 MHz, METHANOL-d₄) δ=−76.924, −120.629.

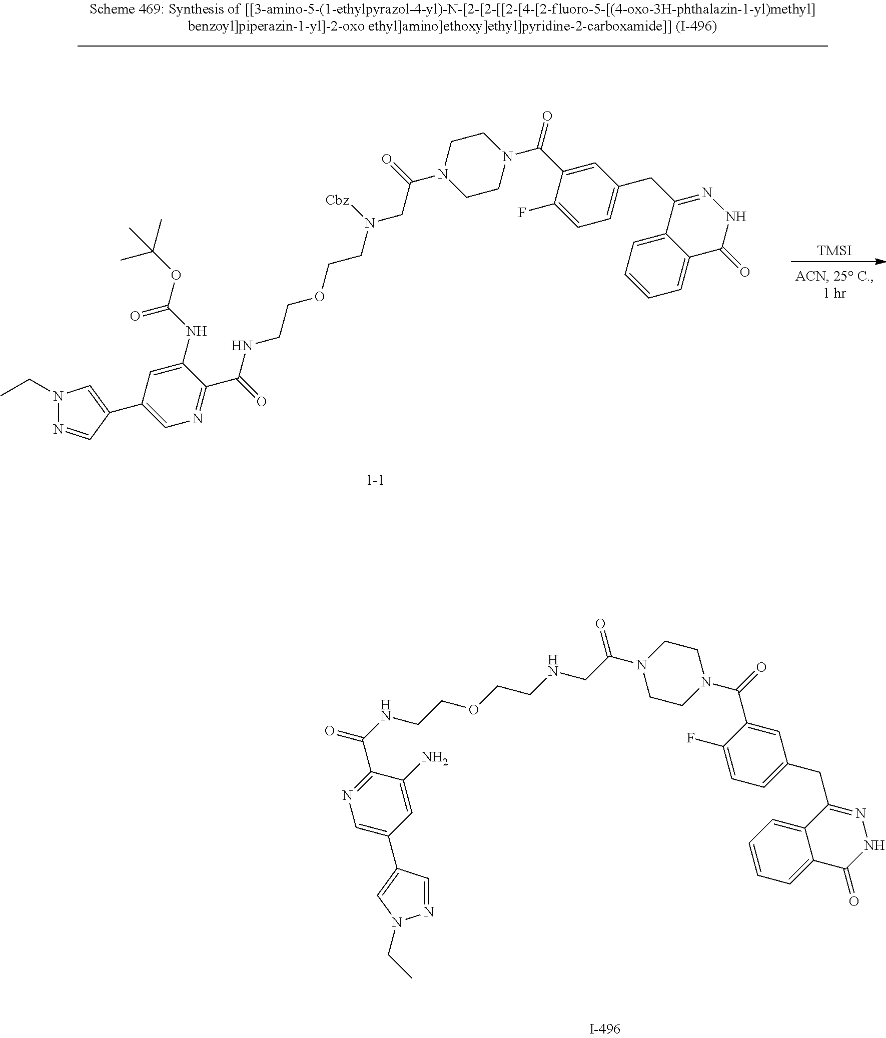 [SMILES: "TMSI" ; CCn1cc(-c2cnc(C(=O)NCCOCCN(CC(=O)N3CCN(C(=O)c4cc(Cc5n[nH]c(=O)c6ccccc56)ccc4F)CC3)C(=O)OCc3ccccc3)c(NC(=O)OC(C)(C)C)c2)cn1 ; CCn1cc(-c2cnc(C(=O)NCCOCCNCC(=O)N3CCN(C(=O)c4cc(Cc5n[nH]c(=O)c6ccccc56)ccc4F)CC3)c(N)c2)cn1]

To a solution of Intermediate 1-1 (80 mg, 83.42 μmol, 1 eq) in MeCN (0.8 mL) was added TMSI (50.07 mg, 250.25 μmol, 34.06 μL, 3 eq). The mixture was stirred at 25° C. for 1 hr. The reaction mixture was diluted with H₂O (1 mL), filtered and extracted with DCM (3 mL*3). The combined organic layers were dried over Na₂SO₄, filtered and concentrated under reduced pressure to give a residue. The crude product was purified by reversed phase chromatography (0.1% TFA condition) followed by lyophilization to give product. I-496 (8.64 mg, 10.19 μmol, 12.22% yield, 98.94% purity, TFA salt) was obtained as a white solid. LCMS (Method D): Retention time: 0.312 min, [M+H]⁺=725.3. ¹H NMR (400 MHz, METHANOL-d₄) δ=8.40-8.34 (m, 1H), 8.11-8.02 (m, 2H), 7.96-7.92 (m, 1H), 7.89-7.81 (m, 3H), 7.32-7.50 (m, 1H), 7.32 (t, J=6.0 Hz, 1H), 7.27 (d, J=9.2 Hz, 1H), 7.16 (t, J=8.8 Hz, 1H), 4.38 (s, 2H), 4.26-4.18 (m, 2H), 4.14 (s, 1H), 4.08 (s, 1H), 3.81 (s, 2H), 3.78-3.64 (m, 6H), 3.63-3.58 (m, 2H), 3.50 (d, J=4.8 Hz, 1H), 3.43 (s, 1H), 3.30-3.25 (m, 4H), 1.47-1.48 (m, 3H). ¹⁹F NMR (377 MHz, METHANOL-d₄) δ=−77.061, −120.657.

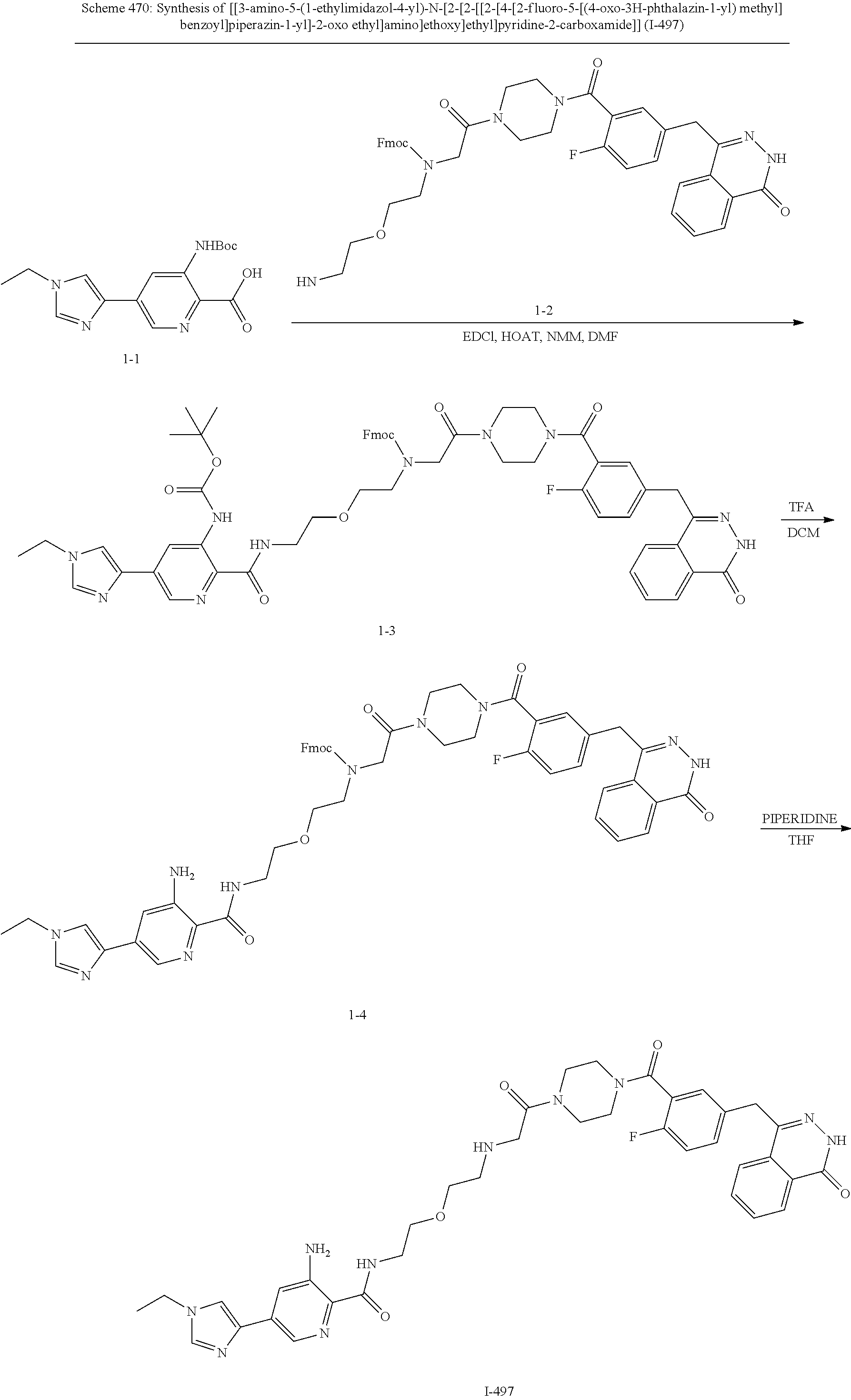

### Step 1: Synthesis of Intermediate 1-3

To a solution of Intermediate 1-1 (100 mg, 300.88 μmol, 1 eq) in DMF (1 mL) was added HOAT (40.95 mg, 300.88 μmol, 42.09 μL, 1 eq), EDCI (173.04 mg, 902.65 μmol, 3 eq), NMM (152.17 mg, 1.50 mmol, 165.40 μL, 5 eq) and Intermediate 1-2 (277.75 mg, 361.06 μmol, 1.2 eq, HCl salt). The mixture was stirred at 25° C. for 12 hr. The reaction mixture was concentrated under vacuum to afford the crude product. The crude product was purified by reversed phase chromatography (0.1% NH₃·H₂O) and lyophilized to give Intermediate 1-3 (30 mg, 28.65 μmol, 9.52% yield) as a white solid. LCMS (Method F): Retention time: 0.703 min, [M+H]⁺=1047.5.

### Step 2: Synthesis of Intermediate 1-4

To a solution of Intermediate 1-3 (30 mg, 28.65 μmol, 1 eq) in DCM (0.02 mL) was added TFA (15.35 mg, 134.62 μmol, 0.01 mL, 4.70 eq). The mixture was stirred at 25° C. for 1 hr. The reaction mixture was concentrated under vacuum to afford the crude product. The crude product was used in the next step without further purification. Intermediate 1-3 (30 mg, crude, TFA salt) was obtained as a brown oil. LCMS (Method D): Retention time: 0.401 min, [M+H]⁺=947.4.

### Step 3: Synthesis of I-497

To a solution of Intermediate 1-4 (30 mg, 28.27 μmol, 1 eq, TFA salt) in THF (0.3 mL) was added piperidine (51.73 mg, 607.56 μmol, 0.06 mL, 21.49 eq). The mixture was stirred at 25° C. for 1 hr. The reaction mixture was concentrated under vacuum to afford the crude product. The crude product was purified by reversed phase chromatography (0.1% FA condition) and lyophilized to give I-497 (3.39 mg, 3.87 μmol, 13.68% yield, 95.739% purity, TFA salt) was obtained as a white solid. LCMS (Method D): Retention time: 0.261 min, [M+H]⁺=725.2. ¹H NMR (400 MHz, METHANOL-d₄) δ=8.51-8.40 (m, 1H), 8.37 (d, J=8.0 Hz, 1H), 8.20 (d, J=12.4 Hz, 1H), 7.97-7.91 (m, 1H), 7.90-7.80 (m, 2H), 7.78 (d, J=4.4 Hz, 1H), 7.66 (s, 1H), 7.50 (d, J=5.2 Hz, 1H), 7.41 (d, J=9.2 Hz, 1H), 7.33 (d, J=5.6 Hz, 1H), 7.18-7.13 (m, 1H), 4.38 (br s, 2H), 4.17-4.05 (m, 3H), 4.01 (s, 1H), 3.79 (s, 2H), 3.76-3.64 (m, 5H), 3.63-3.41 (m, 5H), 3.24 (s, 3H), 3.16-3.11 (m, 1H), 1.53-1.42 (m, 3H). ¹⁹F NMR (377 MHz, METHANOL-d₄) δ=−120.517.

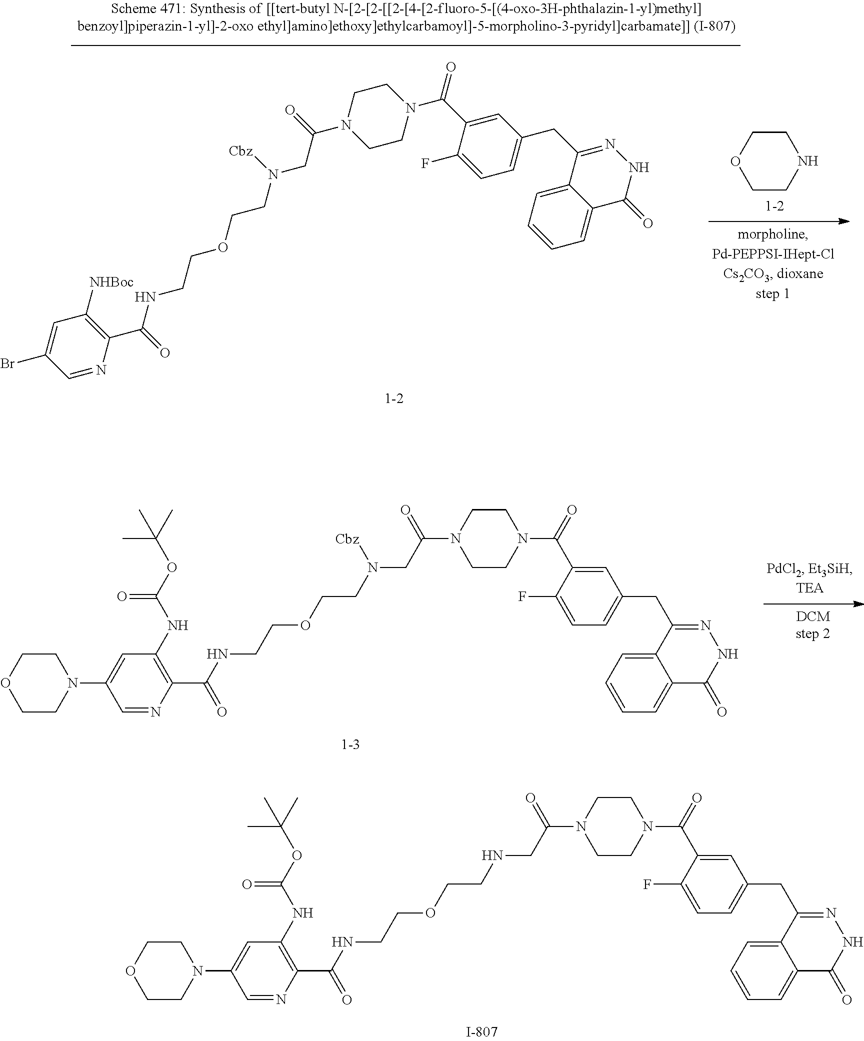 [SMILES: CC(C)(C)OC(=O)Nc1cc(Br)cnc1C(=O)NCCOCCN(CC(=O)N1CCN(C(=O)c2cc(Cc3n[nH]c(=O)c4ccccc34)ccc2F)CC1)C(=O)OCc1ccccc1 ; C1COCCN1 ; CC(C)(C)OC(=O)Nc1cc(N2CCOCC2)cnc1C(=O)NCCOCCN(CC(=O)N1CCN(C(=O)c2cc(Cc3n[nH]c(=O)c4ccccc34)ccc2F)CC1)C(=O)OCc1ccccc1 ; CC(C)(C)OC(=O)Nc1cc(N2CCOCC2)cnc1C(=O)NCCOCCNCC(=O)N1CCN(C(=O)c2cc(Cc3n[nH]c(=O)c4ccccc34)ccc2F)CC1]

### Step 1: Synthesis of Intermediate 1-3

A mixture of Intermediate 1-1 (100 mg, 105.95 μmol, 0.6 eq), morpholine (61.54 mg, 706.36 μmol, 62.16 μL, 4 eq), Cs₂CO₃ (57.54 mg, 176.59 μmol, 1 eq), Intermediate 1-2 (17.18 mg, 17.66 μmol, 0.1 eq) and Pd-PEPPSI-IHept-Cl (17.18 mg, 17.66 μmol, 0.1 eq) in dioxane (1 mL) was degassed and purged with N₂ 3 times, and then the mixture was stirred at 90° C. for 16 hr under N₂ atmosphere. The mixture was diluted with water (1 mL) and extracted with EA (2 mL*3). The combined organic layers were dried over sodium sulfate, filtered and concentrated under reduced pressure to give a residue. The residue was purified by reversed-phase chromatography (0.1% FA condition) and the eluent was lyophilized to give product. Intermediate 1-3 (150 mg, 157.89 μmol, 44.71% yield, 100% purity) was obtained as a white solid. LCMS (Method D): Retention time: 0.544 min, [M+H]⁺=950.4.

### Step 2: Synthesis of I-807

To a solution of Intermediate 1-3 (50 mg, 52.63 μmol, 1 eq) in DCM (0.5 mL) was added PdCl₂ (4.67 mg, 26.32 μmol, 0.5 eq) and TEA (15.98 mg, 157.89 μmol, 21.98 μL, 3 eq). The reaction mixture was degassed with N₂. Then Et₃SiH (73.44 mg, 631.57 μmol, 100.87 μL, 12 eq) was added. The reaction mixture was stirred at 25° C. for 1 hr. Then the reaction mixture was diluted with water (5 mL) and extracted with DCM (5 mL*3), the combined organic phase was dried by Na₂SO₄, and concentrated under reduced pressure to give a residue. The crude was purified by prep-HPLC (column: CD01-Phenomenex luna C18 150*25*10 um; mobile phase: [water (FA)-ACN]; gradient: 18%-48% B over 10 min) and the eluent was concentrated to remove ACN and lyophilized to give product. I-807 (35 mg, 42.90 μmol, 81.51% yield, 100% purity) was obtained as a white solid. LCMS (Method D): Retention time: 0.384 min, [M+H]⁺=816.3. ¹H NMR (400 MHz, CHLOROFORM-d₄) δ=11.27 (d, J=6.8 Hz, 1H), 10.64-10.23 (m, 1H), 8.50-8.42 (m, 1H), 8.35 (t, J=5.2 Hz, 1H), 8.28 (s, 1H), 7.86-7.65 (m, 4H), 7.33 (d, J=5.2 Hz, 2H), 7.06 (t, J=8.0 Hz, 1H), 4.28 (s, 2H), 3.88-3.83 (m, 4H), 3.82-3.72 (m, 2H), 3.70-3.58 (m, 9H), 3.50 (s, 1H), 3.40 (d, J=0.8 Hz, 1H), 3.37-3.23 (m, 6H), 2.95 (d, J=4.0 Hz, 2H), 1.51 (s, 9H). ¹⁹F NMR (377 MHz, METHANOL-d₄) δ=−117.603.

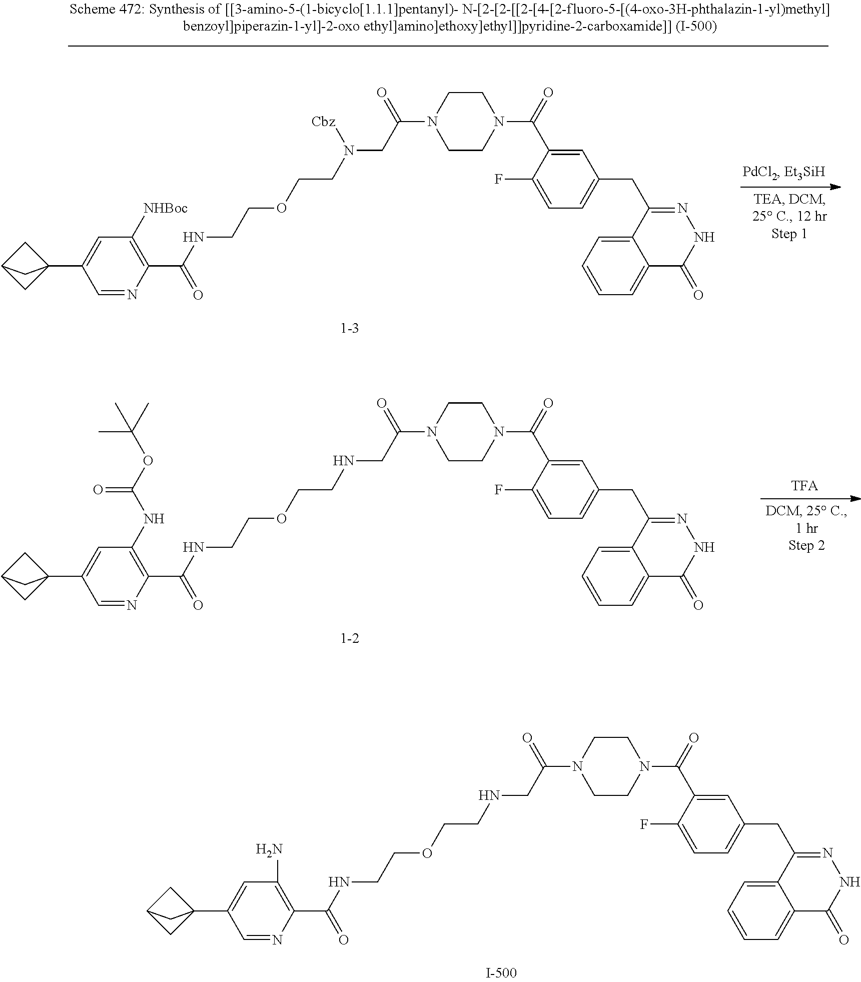 [SMILES: CC(C)(C)OC(=O)Nc1cc(C23CC(C2)C3)cnc1C(=O)NCCOCCN(CC(=O)N1CCN(C(=O)c2cc(Cc3n[nH]c(=O)c4ccccc34)ccc2F)CC1)C(=O)OCc1ccccc1 ; CC(C)(C)OC(=O)Nc1cc(C23CC(C2)C3)cnc1C(=O)NCCOCCNCC(=O)N1CCN(C(=O)c2cc(Cc3n[nH]c(=O)c4ccccc34)ccc2F)CC1 ; Nc1cc(C23CC(C2)C3)cnc1C(=O)NCCOCCNCC(=O)N1CCN(C(=O)c2cc(Cc3n[nH]c(=O)c4ccccc34)ccc2F)CC1]

### Step 1: Synthesis of Intermediate 1-2

To a solution of Intermediate 1-1 (50 mg, 53.70 μmol, 1 eq) in DCM (0.5 mL) was added PdCl₂ (952.33 g, 5.37 μmol, 0.1 eq) and TEA (16.30 mg, 161.11 μmol, 22.43 μL, 3 eq). The suspension was degassed and purged with N₂ 3 times. Then Et₃SiH (31.22 mg, 268.52 μmol, 42.89 μL, 5 eq) was added into the mixture and it was stirred at 25° C. for 1 hr. The mixture was concentrated under reduced pressure to give a residue. The crude product was purified by reversed phase chromatography (0.1% FA condition). The eluent was concentrated under reduced pressure to remove ACN and then lyophilized to give Intermediate 1-2 (40 mg, 50.20 μmol, 93.47% yield), which was obtained as a yellow solid. LCMS (Method D): Retention time: 0.432 min, [M+H]⁺=797.3.

### Step 2: Synthesis of I-500

To a solution of Intermediate 1-2 (40 mg, 50.20 μmol, 1 eq) in DCM (1 mL) was added TFA (306.99 mg, 2.69 mmol, 199.99 μL, 53.64 eq). The mixture was stirred at 25° C. for 1 hr. The mixture was concentrated under reduced pressure to give a residue. The crude product was purified by reversed phase chromatography (0.1% TFA condition). The eluent was concentrated under vacuum to remove ACN and then lyophilized to give I-500 (20.44 mg, 24.31 μmol, 48.43% yield, 96.438% purity, TFA salt) as a yellow solid. LCMS (Method D): Retention time: 0.354 min, [M+H]⁺=697.2. ¹H NMR (400 MHz, METHANOL-d₄) δ=8.36 (d, J=7.6 Hz, 1H), 7.93 (s, 1H), 7.90-7.78 (m, 2H), 7.68 (d, J=10.4 Hz, 1H), 7.51 (s, 1H), 7.36 (s, 1H), 7.19-7.13 (m, 1H), 6.95 (s, 1H), 4.39 (s, 2H), 4.15 (s, 1H), 4.09 (s, 1H), 3.82-3.65 (m, 7H), 3.59 (s, 2H), 3.52 (s, 1H), 3.44 (s, 1H), 3.40-3.32 (m, 2H), 3.30-3.14 (m, 3H), 2.55 (s, 1H), 2.09 (s, 6H). ¹⁹F NMR (377 MHz, METHANOL-d₄) δ=−77.179, 120.607.

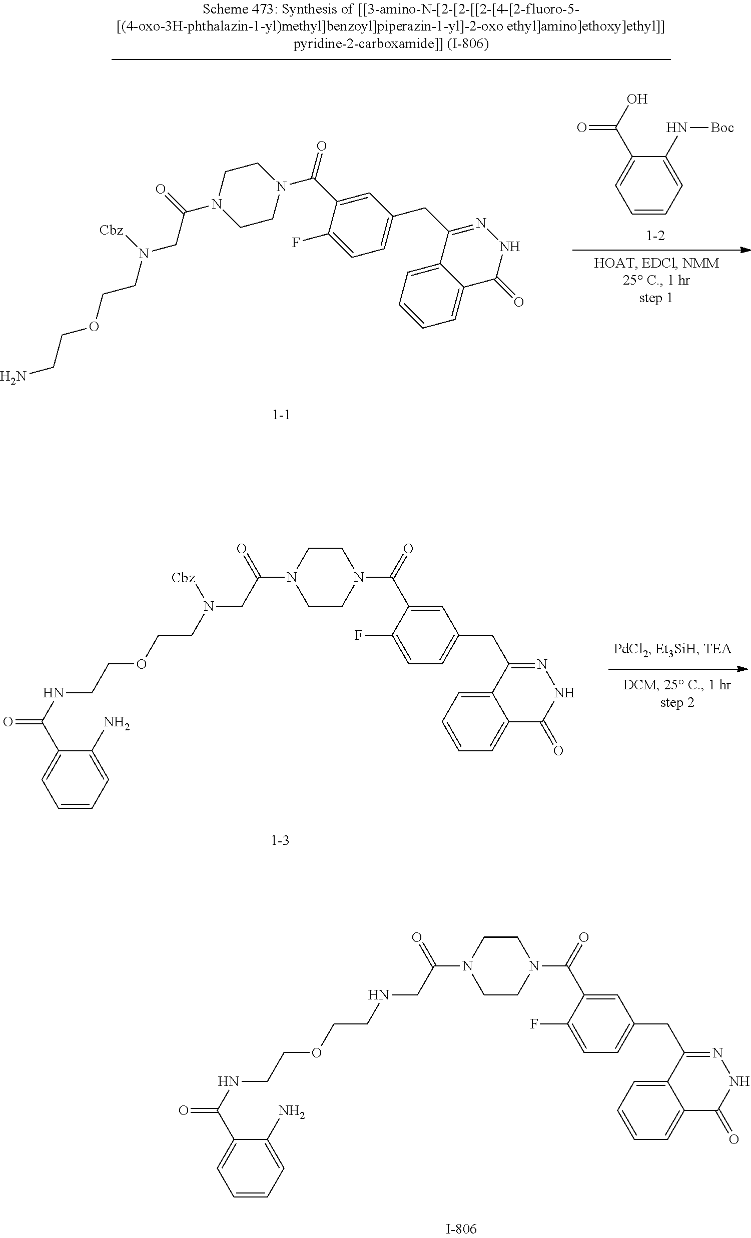 [SMILES: NCCOCCN(CC(=O)N1CCN(C(=O)c2cc(Cc3n[nH]c(=O)c4ccccc34)ccc2F)CC1)C(=O)OCc1ccccc1 ; CC(C)(C)OC(=O)Nc1ccccc1C(=O)O ; Nc1ccccc1C(=O)NCCOCCN(CC(=O)N1CCN(C(=O)c2cc(Cc3n[nH]c(=O)c4ccccc34)ccc2F)CC1)C(=O)OCc1ccccc1 ; Nc1ccccc1C(=O)NCCOCCNCC(=O)N1CCN(C(=O)c2cc(Cc3n[nH]c(=O)c4ccccc34)ccc2F)CC1]

### Step 1: Synthesis of Intermediate 1-3

To a solution of Intermediate 1-1 (500 mg, 734.05 μmol, 1 eq, HCl salt) in DMF (5 mL) was added Intermediate 1-2 (174.88 mg, 734.05 μmol, 1 eq), EDCI (422.15 mg, 2.20 mmol, 3 eq), HOAt (99.91 mg, 734.05 μmol, 102.68 μL, 1 eq) and NMM (371.24 mg, 3.67 mmol, 403.52 μL, 5 eq). The mixture was stirred at 25° C. for 1 hr. The mixture was concentrated under reduced pressure to give a crude product. The crude product was purified by reversed phase chromatography (0.10% FA condition) and concentrated under vacuum to give the product. Intermediate 1-3 (160 mg, 200.97 μmol, 27.38% yield, 96.064% purity) was obtained as a yellow solid. LCMS (Method D): Retention time: 0.426 min, [M+H]⁺=765.4.

### Step 2: Synthesis of I-806

To a solution of Intermediate 1-3 (80 mg, 104.60 μmol, 1 eq) in DCM (1 mL) was added PdCl₂ (9.27 mg, 52.30 μmol, 0.5 eq) and TEA (31.75 mg, 313.81 μmol, 43.68 μL, 3 eq). The reaction mixture was degassed with N₂. Then Et₃SiH (145.95 mg, 1.26 mmol, 200.49 μL, 12 eq) was added. The mixture was stirred at 25° C. for 1 hr. The mixture was concentrated under N₂ flow to give a crude product. The crude product was purified by prep-HPLC (column: CD01-Phenomenex luna C18 150*25*10 um; mobile phase: [water(FA)-ACN]; gradient: 7%-37% B over 10 min) and lyophilized to give the product. I-806 (26.97 mg, 39.77 μmol, 38.02% yield, 99.777% purity, FA salt) was obtained as a white solid. LCMS (Method D): Retention time: 0.295 min, [M+H]⁺=631.3. ¹H NMR (400 MHz, METHANOL-d₄) δ =8.45 (s, 1H), 8.37 (d, J=7.6 Hz, 1H), 7.98-7.92 (m, 1H), 7.90-7.76 (m, 3H), 7.51 (d, J=2.8 Hz, 1H), 7.39-7.33 (m, 1H), 7.22-7.09 (m, 3H), 4.39 (s, 2H), 4.07 (s, 1H), 3.99 (s, 1H), 3.80-3.73 (m, 4H), 3.69 (d, J=4.4 Hz, 3H), 3.63-3.57 (m, 2H), 3.57-3.41 (m, 3H), 3.29-3.17 (m, 4H). ¹⁹F NMR (377 MHz, METHANOL-d₄) δ=−120.620.

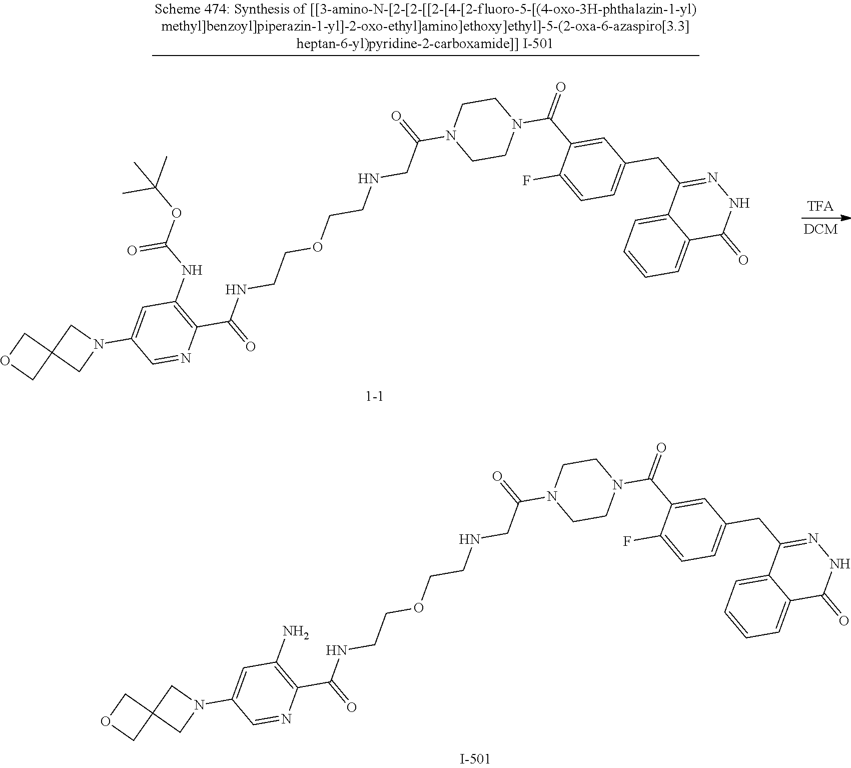 [SMILES: CC(C)(C)OC(=O)Nc1cc(N2CC3(COC3)C2)cnc1C(=O)NCCOCCNCC(=O)N1CCN(C(=O)c2cc(Cc3n[nH]c(=O)c4ccccc34)ccc2F)CC1 ; Nc1cc(N2CC3(COC3)C2)cnc1C(=O)NCCOCCNCC(=O)N1CCN(C(=O)c2cc(Cc3n[nH]c(=O)c4ccccc34)ccc2F)CC1]

A mixture of Intermediate 1-1 (40 mg, 48.32 μmol, 1 eq) in TFA (0.5 mL) and DCM (0.5 mL) was stirred at 20° C. for 1 hour. The reaction mixture was concentrated under reduced pressure to give a residue. The crude product was purified by reversed-phase chromatography (0.1% NH₃·H₂O condition). The eluent was concentrated under reduced pressure to remove MeCN and then lyophilized to give I-501 (16.99 mg, 23.15 μmol, 47.91% yield, 99.1490% purity) as a white solid. LCMS (Method D): Retention time: 0.312 min, [M+H]⁺=728.4. ¹H NMR (400 MHz, METHANOL-d₄) δ=8.40-8.33 (m, 1H), 7.96-7.91 (m, 1H), 7.89-7.80 (m, 2H), 7.52-7.45 (m, 1H), 7.35 (d, J=5.2 Hz, 1H), 7.20-7.10 (m, 2H), 5.99 (d, J=9.6 Hz, 1H), 4.81 (d, J=9.6 Hz, 4H), 4.38 (s, 2H), 4.07 (s, 4H), 3.71 (s, 2H), 3.67-3.58 (m, 5H), 3.55-3.46 (m, 6H), 3.37-3.33 (m, 1H), 3.29 (s, 2H), 2.81 (d, J=4.8 Hz, 2H). ¹⁹F NMR (377 MHz, METHANOL-d₄) δ =−120.68.

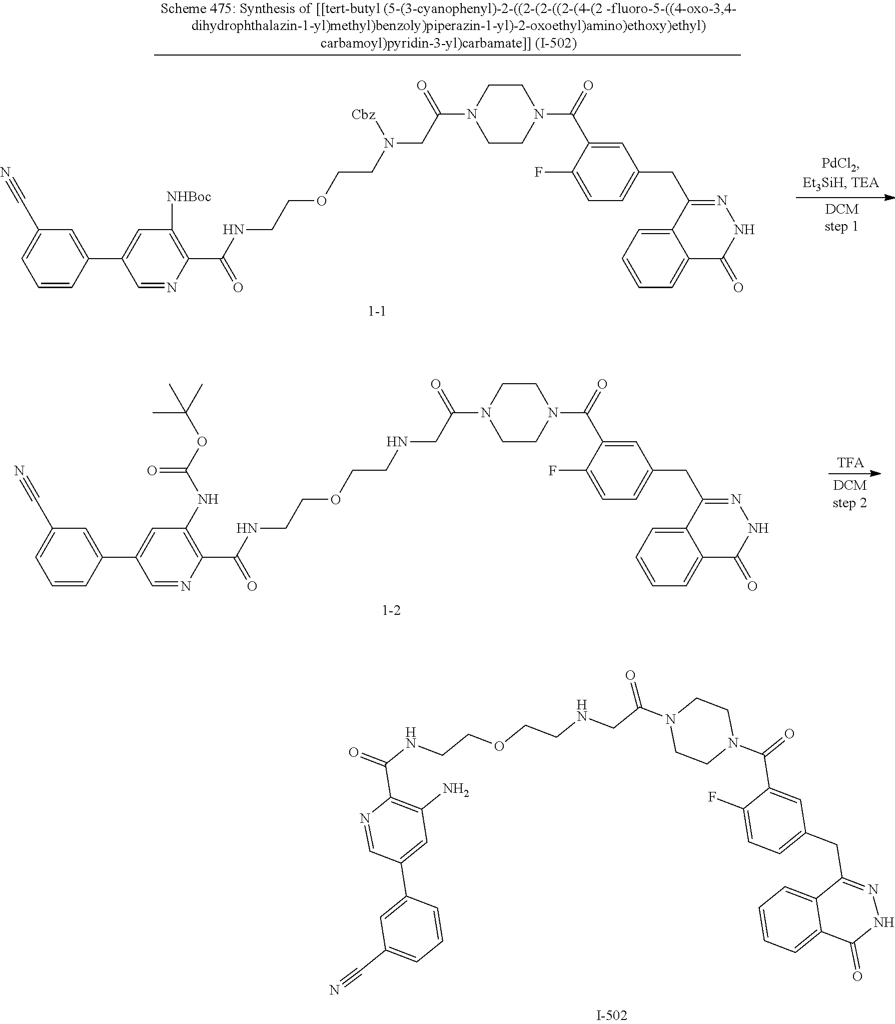 [SMILES: CC(C)(C)OC(=O)Nc1cc(-c2cccc(C#N)c2)cnc1C(=O)NCCOCCN(CC(=O)N1CCN(C(=O)c2cc(Cc3n[nH]c(=O)c4ccccc34)ccc2F)CC1)C(=O)OCc1ccccc1 ; CC(C)(C)OC(=O)Nc1cc(-c2cccc(C#N)c2)cnc1C(=O)NCCOCCNCC(=O)N1CCN(C(=O)c2cc(Cc3n[nH]c(=O)c4ccccc34)ccc2F)CC1 ; N#Cc1cccc(-c2cnc(C(=O)NCCOCCNCC(=O)N3CCN(C(=O)c4cc(Cc5n[nH]c(=O)c6ccccc56)ccc4F)CC3)c(N)c2)c1]

### Step 1: Synthesis of Intermediate 1-2

To a solution of Intermediate 1-1 (100 mg, 103.52 μmol, 1 eq) in DCM (1 mL) was added PdCl₂ (9.18 mg, 51.76 μmol, 0.5 eq), Et₃SiH (144.44 mg, 1.24 mmol, 198.41 μL, 12 eq) and TEA (31.42 mg, 310.55 μmol, 43.22 μL, 3 eq). The mixture was stirred at 25° C. for 1 hr. The reaction mixture was concentrated under reduced pressure to give a residue. The crude product was purified by reversed phase chromatography (0.1% TFA condition), the eluent was concentrated to remove ACN and lyophilized to give product. Intermediate 1-2 (80 mg, 96.17 μmol, 92.90% yield, 98.217% purity) was obtained as a white solid. LCMS (Method D): Retention time: 0.394 min, [M+H]⁺=832.3.

### Step 2: Synthesis of I-502

To a solution of Intermediate 1-2 (40 mg, 48.08 μmol, 1 eq) in DCM (0.3 mL) was added TFA (153.50 mg, 1.35 mmol, 0.1 mL, 28.00 eq). The mixture was stirred at 25° C. for 1 hr. The reaction mixture was concentrated under reduced pressure to give a residue. The crude product was purified by reversed phase chromatography (0.1% TFA condition), the eluent was concentrated to remove ACN and lyophilized to give product. I-502 (20.56 mg, 23.40 μmol, 48.67% yield, 96.262% purity, TFA salt) was obtained as a white solid. LCMS: Retention time: 0.331 min, [M+H]⁺=732.2. ¹H NMR (400 MHz, METHANOL-d₄) δ =8.43-8.31 (m, 1H), 8.13-8.08 (m, 1H), 8.01 (d, J=4.0 Hz, 1H), 7.96-7.93 (m, 2H), 7.90-7.73 (m, 3H), 7.70-7.64 (m, 1H), 7.55-7.46 (m, 1H), 7.41-7.38 (m, 1H), 7.35-7.31 (m, 1H), 7.18-7.13 (m, 1H), 4.38 (s, 2H), 4.22-4.03 (m, 2H), 3.83 (s, 2H), 3.78-3.66 (m, 5H), 3.66-3.58 (m, 2H), 3.52 (t, J=5.2 Hz, 1H), 3.49-3.44 (m, 1H), 3.31 (s, 5H). ¹⁹F NMR (377 MHz, METHANOL-d₄) δ=−77.082, −120.637.

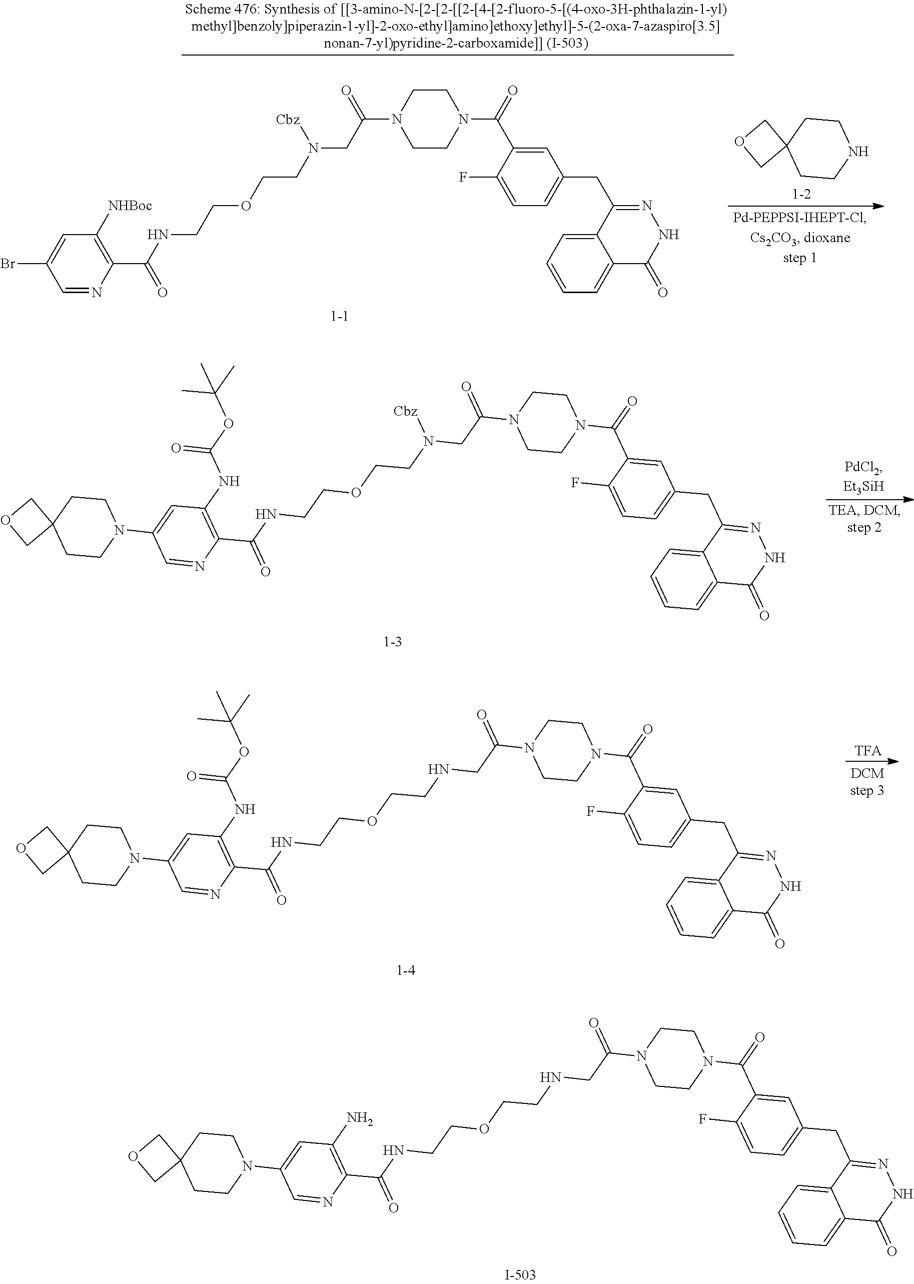 [SMILES: CC(C)(C)OC(=O)Nc1cc(Br)cnc1C(=O)NCCOCCN(CC(=O)N1CCN(C(=O)c2cc(Cc3n[nH]c(=O)c4ccccc34)ccc2F)CC1)C(=O)OCc1ccccc1 ; C1CC2(CCN1)COC2 ; CC(C)(C)OC(=O)Nc1cc(N2CCC3(CC2)COC3)cnc1C(=O)NCCOCCN(CC(=O)N1CCN(C(=O)c2cc(Cc3n[nH]c(=O)c4ccccc34)ccc2F)CC1)C(=O)OCc1ccccc1 ; CC(C)(C)OC(=O)Nc1cc(N2CCC3(CC2)COC3)cnc1C(=O)NCCOCCNCC(=O)N1CCN(C(=O)c2cc(Cc3n[nH]c(=O)c4ccccc34)ccc2F)CC1 ; Nc1cc(N2CCC3(CC2)COC3)cnc1C(=O)NCCOCCNCC(=O)N1CCN(C(=O)c2cc(Cc3n[nH]c(=O)c4ccccc34)ccc2F)CC1]

### Step 1: Synthesis of Intermediate 1-3

To a solution of Intermediate 1-1 (500 mg, 529.77 μmol, 1 eq) and Intermediate 1-2 (202.13 mg, 1.59 mmol, 3 eq) in dioxane (6 mL) was added Cs₂CO₃ (517.82 mg, 1.59 mmol, 3 eq) and Pd-PEPPSI-IHEPT-Cl (51.53 mg, 52.98 μmol, 0.1 eq), then the mixture was stirred at 90° C. for 1 hour under N₂. The reaction mixture was diluted with water (8 mL) and extracted with EA (10 mL*2). The combined organic layers were concentrated under reduced pressure to give a residue. The crude product was purified by reversed phase chromatography (0.1% FA condition). The eluent was concentrated under reduced pressure to remove ACN and then lyophilized to give the product. Intermediate 1-3 (410 mg, 386.26 μmol, 72.91% yield, 93.275% purity) was obtained as a yellow solid. LCMS (Method D): Retention time: 0.543 min, [M+H]⁺=990.4.

### Step 2: Synthesis of Intermediate 1-4

To a solution of Intermediate 1-3 (150 mg, 151.50 gmol, 1 eq) in DCM (1.5 mL) was added PdCl₂ (5.37 mg, 30.30 μmol, 0.2 eq) and TEA (30.66 mg, 303.00 μmol, 42.17 μL, 2 eq). The reaction mixture was degassed with N₂. Then a mixture of Et₃SiH (70.46 mg, 606.01 μmol, 96.79 μL, 4 eq) in DCM (1 mL) was added dropwise at 10° C. Then the mixture was stirred at 25° C. for 2 hours. The reaction mixture was concentrated under reduced pressure to give a residue. Intermediate 1-4 (110 mg, 128.51 μmol, 84.83% yield) was obtained as a white solid. LCMS (Method D): Retention time: 0.394 min, [M+H]⁺=856.5.

### Step 3: Synthesis of I-503

To a solution of Intermediate 1-4 (50 mg, 58.41 μmol, 1 eq) in DCM (1 mL) was added TFA (0.2 mL), then the mixture was stirred at 25° C. for 0.5 hour. To the reaction mixture was added NaHCO₃ aq. (about 6 mL) to adjust the pH to 7-8, and the mixture was extracted with DCM 15 mL (5 mL*3). The combined organic layers were concentrated under reduced pressure to give a residue. The crude product was purified by reversed phase chromatography (0.10% NH₃·H₂O condition). The eluent was concentrated under reduced pressure to remove ACN and then lyophilized to give the product. I-503 (17.68 mg, 22.88 μmol, 39.17% yield, 97.819% purity) was obtained as a white solid. LCMS (Method D): Retention time: 0.317 min, [M+H]⁺=756.4. ¹H NMR (400 MHz, METHANOL-d₄) δ=8.37 (d, J=7.6 Hz, 1H), 7.97-7.91 (m, 1H), 7.89-7.80 (m, 2H), 7.62 (d, J=14.4 Hz, 1H), 7.52-7.45 (m, 1H), 7.35 (d, J=5.6 Hz, 1H), 7.19-7.13 (m, 1H), 6.47 (d, J=10.8 Hz, 1H), 4.47 (d, J=6.0 Hz, 4H), 4.38 (s, 2H), 3.75-3.58 (m, 8H), 3.57-3.44 (m, 7H), 3.29-3.25 (m, 2H), 3.21 (s, 4H), 2.82 (d, J=4.4 Hz, 1H), 1.94 (s, 4H). ¹⁹F NMR (377 MHz, METHANOL-d₄) δ=−120.673.

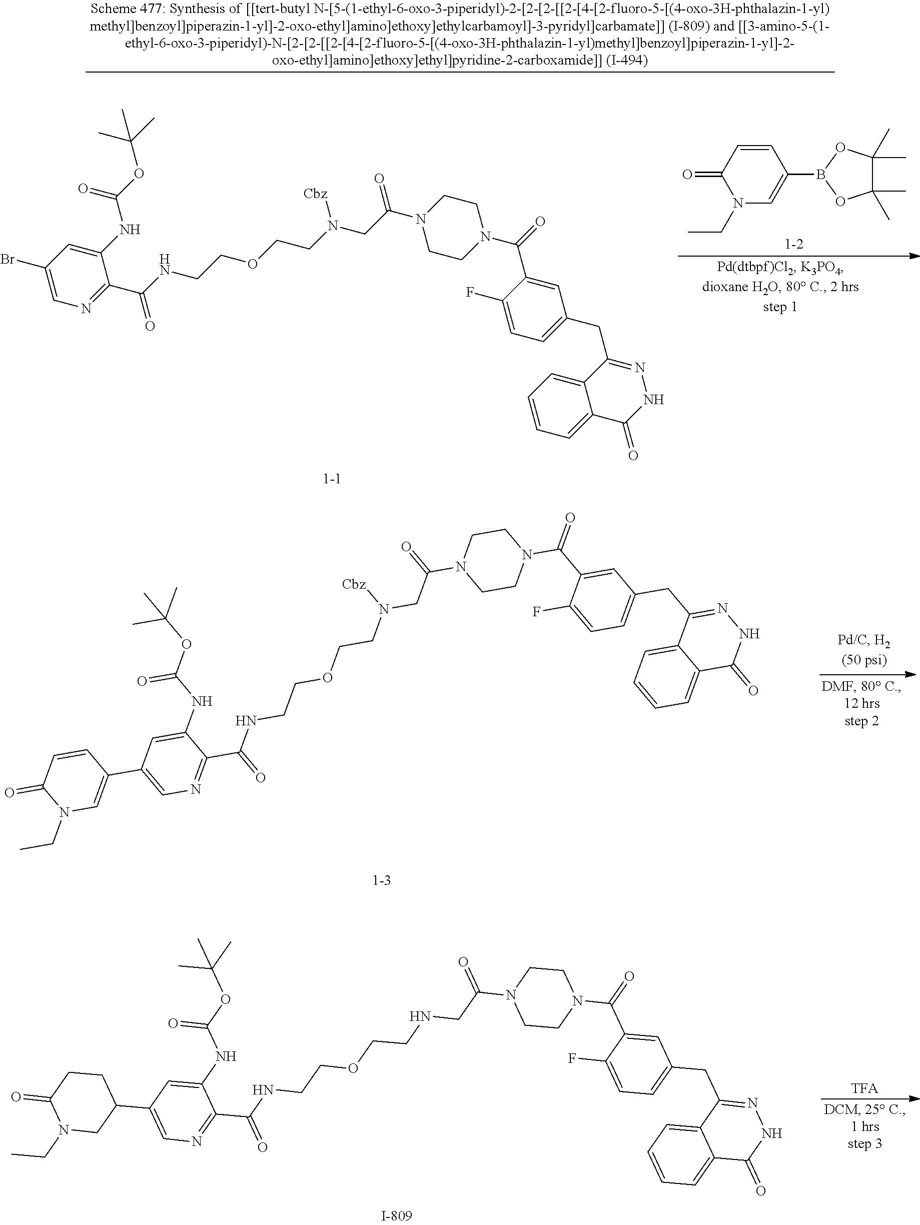 [SMILES: CC(C)(C)OC(=O)Nc1cc(Br)cnc1C(=O)NCCOCCN(CC(=O)N1CCN(C(=O)c2cc(Cc3n[nH]c(=O)c4ccccc34)ccc2F)CC1)C(=O)OCc1ccccc1 ; CCn1cc(B2OC(C)(C)C(C)(C)O2)ccc1=O ; CCn1cc(-c2cnc(C(=O)NCCOCCN(CC(=O)N3CCN(C(=O)c4cc(Cc5n[nH]c(=O)c6ccccc56)ccc4F)CC3)C(=O)OCc3ccccc3)c(NC(=O)OC(C)(C)C)c2)ccc1=O ; CCN1CC(c2cnc(C(=O)NCCOCCNCC(=O)N3CCN(C(=O)c4cc(Cc5n[nH]c(=O)c6ccccc56)ccc4F)CC3)c(NC(=O)OC(C)(C)C)c2)CCC1=O]

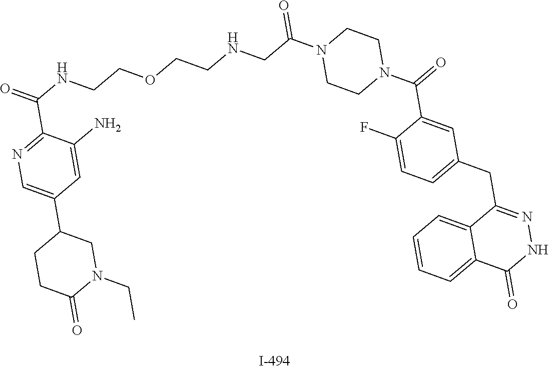 [SMILES: CCN1CC(c2cnc(C(=O)NCCOCCNCC(=O)N3CCN(C(=O)c4cc(Cc5n[nH]c(=O)c6ccccc56)ccc4F)CC3)c(N)c2)CCC1=O]

### Step 1: Synthesis of Intermediate 1-3

A mixture of Intermediate 1-1 (250 mg, 264.88 μmol, 1 eq), Intermediate 1-2 (98.98 mg, 397.33 μmol, 1.5 eq), K₃PO₄ (168.68 mg, 794.65 μmol, 3 eq) and Pd(dtbpf)Cl₂ (17.26 mg, 26.49 μmol, 0.1 eq) in dioxane (2.5 mL) and H₂O (0.5 mL) was degassed and purged with N₂ 3 times, and then the mixture was stirred at 80° C. for 2 hr under N₂ atmosphere. The mixture was diluted with H₂O (5 mL) and extracted with EA (5 mL*3), dried over Na₂SO₄, filtered and concentrated under reduced pressure to give a residue. The crude product was purified by flash silica gel chromatography (ISCO; 20 g SepaFlash Silica Flash Column, Eluent of 0˜100% dichloromethane/Methanol @60 mL/min). The eluent was concentrated under reduced pressure to give product. Intermediate 1-3 (260 mg, 233.29 μmol, 88.07% yield, 88.477% purity) was obtained as a brown solid. LCMS (Method D): Retention time: 0.485 min, [M+H]⁺=986.4.

### Step 2: Synthesis of I-809

A mixture of Intermediate 1-3 (30 mg, 30.42 μmol, 1 eq) and Pd/C (10 mg, 9.40 μmol, 10% purity, 3.09e−1 eq) in MeOH (0.5 mL) was degassed and purged with N₂ 3 times, and then the mixture was stirred at 80° C. for 12 hr under H₂ atmosphere (50 psi). The reaction mixture was diluted with MeOH (3 mL*3), filtered and concentrated under reduced pressure to give a residue. The crude product was purified by reversed phase chromatography (0.1% FA condition) followed by lyophilization to give I-809 (18.04 mg, 19.51 μmol, 64.12% yield, 97.537% purity, FA salt) as a white solid. LCMS (Method D): Retention time: 0.417 min, [M+H]⁺=856.4. SFC: Retention time: 4.120 min, 5.065 min. ¹H NMR (400 MHz, METHANOL-d₄) δ=8.74 (s, 1H), 8.55-8.50 (m, 1H), 8.37 (d, J=7.2 Hz, 1H), 8.21 (d, J=5.2 Hz, 1H), 7.98-7.91 (m, 1H), 7.90-7.78 (m, 2H), 7.49 (d, J=1.2 Hz, 1H), 7.40-7.32 (m, 1H), 7.21-7.10 (m, 1H), 4.38 (s, 2H), 3.73 (s, 2H), 3.66 (s, 6H), 3.59 (d, J=3.6 Hz, 3H), 3.55-3.48 (m, 4H), 3.47-3.42 (m, 2H), 3.39-3.35 (m, 1H), 3.28-3.21 (m, 2H), 2.96-2.83 (m, 2H), 2.49 (s, 2H), 2.16-2.04 (m, 2H), 1.52 (s, 9H), 1.34-1.25 (m, 1H), 1.20-1.11 (m, 3H). ¹⁹F NMR (376 MHz, METHAN OL-d₄) δ=−120.695.

### Step 3: Synthesis of I-494

To a solution of I-809 (40 mg, 46.73 μmol, 1 eq) in DCM (0.5 mL) was added TFA (153.50 mg, 1.35 mmol, 0.1 mL, 28.81 eq). The mixture was stirred at 25° C. for 1 hr. The reaction mixture was concentrated under reduced pressure to give a residue. The crude product was purified by reversed-phase chromatography (0.1% TFA condition) and lyophilized to give product. I-494 (11.58 mg, 12.80 μmol, 27.38% yield, 96.119% purity, TFA salt) was obtained as a yellow solid. LCMS (Method D): Retention time: 0.291 min, [M+H]⁺=756.3. SFC: Retention time: 4.198 min, 5.212 min. ¹H NMR (400 MHz, METHANOL-d₄) δ=8.37 (d, J=8.0 Hz, 1H), 7.98-7.91 (m, 1H), 7.90-7.78 (m, 3H), 7.51 (d, J=6.0 Hz, 1H), 7.35 (d, J=6.0 Hz, 1H), 7.17 (t, J=9.2 Hz, 1H), 7.08 (d, J=5.6 Hz, 1H), 4.39 (s, 2H), 4.19-4.07 (m, 2H), 3.81 (s, 2H), 3.77-3.66 (m, 5H), 3.62-3.57 (m, 2H), 3.54-3.50 (m, 1H), 3.49-3.38 (m, 6H), 3.26 (s, 2H), 3.16-3.07 (m, 1H), 2.50-2.45 (m, 2H), 2.07-1.99 (m, 2H), 1.87-1.47 (m, 1H), 1.46-1.21 (m, 1H), 1.16-1.11 (m, 3H). ¹⁹F NMR (376 MHz, METHANOL-d₄) δ=−76.971, −120.665.

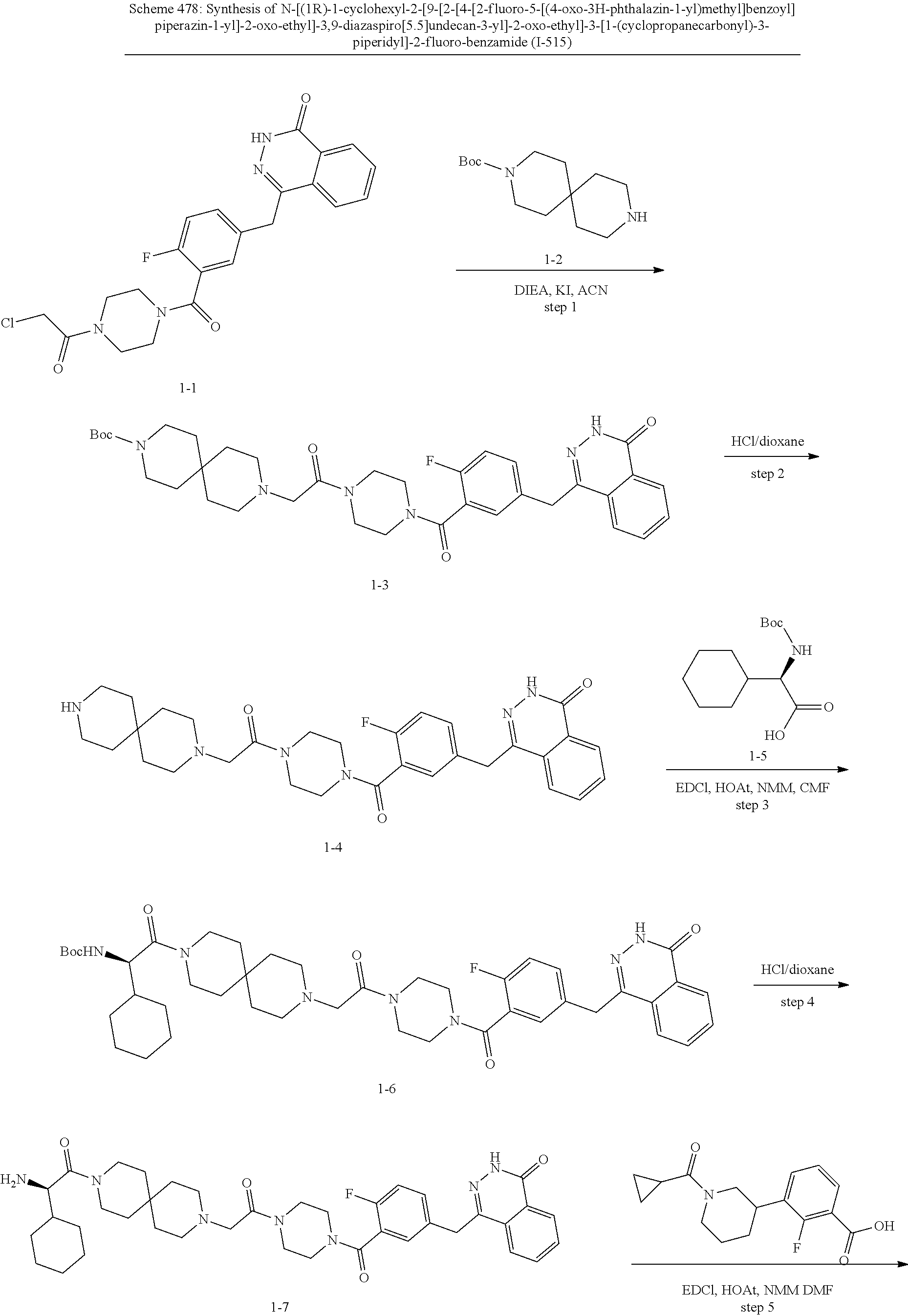

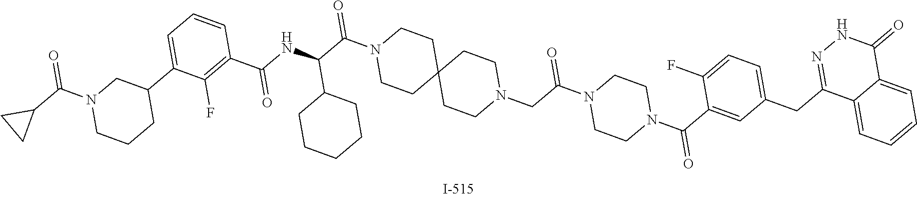 [SMILES: O=C(N[C@@H](C(=O)N1CCC2(CCN(CC(=O)N3CCN(C(=O)c4cc(Cc5n[nH]c(=O)c6ccccc56)ccc4F)CC3)CC2)CC1)C1CCCCC1)c1cccc(C2CCCN(C(=O)C3CC3)C2)c1F]

### Step 1: Synthesis of Intermediate 1-3

To a solution of Intermediate 1-1 (250 mg, 564.50 μmol, 1 eq) in ACN (3 mL) was added Intermediate 1-2 (143.59 mg, 564.50 μmol, 1 eq), DIEA (291.83 mg, 2.26 mmol, 393.30 μL, 4 eq) and KI (374.83 mg, 2.26 mmol, 4 eq). The mixture was stirred at 25° C. for 3 hr. The mixture was diluted with water (5 mL), extracted with EtOAc (3 mL×3) and washed with brine (5 mL). The organic layer was dried with anhydrous Na₂SO₄, filtered and concentrated under vacuum. The residue was purified by column chromatography (SiO₂, Dichloromethane/Methanol=50/1 to 10/1) and concentrated under vacuum. Intermediate 1-3 (438 mg, 601.21 μmol, 106.50% yield, 90.7% purity) was obtained as a light yellow oil. LCMS (Method G): Rt=0.622 min, [M+H]⁺=661.4. ¹H NMR (400 MHz, METHANOL-d₄) δ=8.37 (d, J=8.0 Hz, 1H), 7.98-7.93 (m, 1H), 7.91-7.80 (m, 2H), 7.49 (br s, 1H), 7.40-7.36 (m, 1H), 7.19-7.13 (m, 1H), 5.49 (s, 2H), 4.38 (s, 2H), 3.79 (br d, J=4.0 Hz, 1H), 3.75-3.67 (m, 3H), 3.58-3.49 (m, 2H), 3.38 (br s, 4H), 3.30 (br s, 2H), 2.64-2.43 (m, 4H), 1.62-1.50 (m, 4H), 1.45 (s, 12H), 1.37 (d, J=8.0 Hz, 1H).

### Step 2: Synthesis of Intermediate 1-4

To a solution of Intermediate 1-3 (380 mg, 575.08 μmol, 1 eq) in DCM (4 mL) was added HCl/dioxane (2 M, 8 mL, 27.82 eq). The mixture was stirred at 25° C. for 30 min. The residue was combined with another lot of material (50 mg of Intermediate 1-3 starting material). The reaction mixture was triturated with DCM (5 ml), filtered and the filter cake was washed with DCM (10 mL*2). The filter cake was dried under vacuum. This residue was used in the next step without purification. Intermediate 1-4 (320 mg, crude, HCl salt) as a yellow solid was obtained. LCMS (Method E): Rt=0.347 min, [M+H]⁺=561.3.

### Step 3: Synthesis of Intermediate 1-6

To a solution of Intermediate 1-4 (260 mg, 435.42 μmol, 1 eq, HCl salt), EDCI (166.94 mg, 870.84 μmol, 2 eq), HOAt (59.27 mg, 435.42 μmol, 60.91 μL, 1 eq) and NMM (220.21 mg, 2.18 mmol, 239.36 L, 5 eq) in DMF (3 mL) was added Intermediate 1-5 (112.05 mg, 435.42 μmol, 1 eq). The mixture was stirred at 25° C. for 2 hr. The reaction was combined with another lot of material (50 mg of Intermediate 1-4 starting material). The reaction mixture was triturated with water (5 ml), filtered and the filter cake was washed with water (10 mL*2). The filter cake was dried under vacuum. This residue was used in the next step without further purification. Intermediate 1-6 (310 mg, 355.00 μmol, 81.53% yield, 91.61% purity) was obtained as a light yellow solid. LCMS (Method G): Rt=0.658 min, [M+H]⁺=800.6. SFC: Rt=2.605 min. ¹H NMR (400 MHz, METHANOL-d₄) δ=8.37 (br d, J=8.0 Hz, 1H), 7.96 (br d, J=8.0 Hz, 1H), 7.91-7.81 (m, 2H), 7.49 (br d, J=4.0 Hz, 1H), 7.40-7.35 (m, 1H), 7.20-7.14 (m, 1H), 3.83-3.73 (m, 2H), 3.68 (br d, J=12.0 Hz, 4H), 3.59-3.46 (m, 4H), 3.35 (br s, 3H), 2.65-2.45 (m, 4H), 1.74 (br d, J=12.0 Hz, 4H), 1.69-1.51 (m, 10H), 1.43 (s, 12H), 1.32-1.17 (m, 4H), 1.09-0.97 (m, 2H).

### Step 4: Synthesis of Intermediate 1-7

To a solution of Intermediate 1-6 (150 mg, 187.51 μmol, 1 eq) in DCM (1.5 mL) was added HCl/dioxane (2 M, 3 mL, 32.00 eq). The mixture was stirred at 25° C. for 1 hr. The reaction mixture was concentrated under vacuum. This reaction used in the next step without purification. Intermediate 1-7 (140 mg, HCl salt, crude) was obtained as light yellow oil. LCMS (Method E): Rt=0.377 min, [M+H]⁺=700.4.

### Step 5: Synthesis of I-515

To a solution of Intermediate 1-7 (70 mg, 100.02 μmol, 1 eq, HCl salt), EDCI (38.35 mg, 200.04 μmol, 2 eq), HOAt (13.61 mg, 100.02 μmol, 13.99 μL, 1 eq) and NMM (50.58 mg, 500.10 μmol, 54.98 μL, 5 eq) in DMF (1 mL) was added Intermediate 1-8 (29.14 mg, 100.02 μmol, 1 eq). The mixture was stirred at 25° C. for 2 hr. The mixture was diluted with water (3 ml), and extracted with EtOAc (2 ml*3). The organic layer was concentrated under vacuum. The residue was purified by Prep-HPLC (column: Waters Xbridge 150*25 mm*5 um; mobile phase: [water (NH₄HCO₃)-ACN]; gradient: 40%-70% B over 9 min) and dried by lyophilization. I-515 (27.54 mg, 27.10 μmol, 27.09% yield, 95.76% purity) was obtained as a white solid. LCMS (Method G): Rt=0.667 min, [M+H]⁺=973.7. SFC: Rt=2.899 min, 3.987 min. ¹H NMR (400 MHz, METHANOL-d₄) δ=8.36 (br d, J=8.0 Hz, 1H), 7.97-7.92 (m, 1H), 7.89-7.80 (m, 2H), 7.59-7.47 (m, 3H), 7.41-7.36 (m, 1H), 7.28-7.22 (m, 1H), 7.19-7.13 (m, 1H), 4.95 (br s, 1H), 4.60-4.53 (m, 1H), 4.38 (s, 3H), 3.80-3.63 (m, 7H), 3.56 (br s, 1H), 3.53-3.46 (m, 2H), 3.38-3.31 (m, 3H), 3.23 (br s, 1H), 2.84-2.69 (m, 1H), 2.50 (br d, J=4.0 Hz, 4H), 2.07-1.99 (m, 2H), 1.98-1.89 (m, 2H), 1.89-1.73 (m, 6H), 1.72-1.63 (m, 4H), 1.59 (br d, J=4.0 Hz, 4H), 1.54-1.44 (m, 3H), 1.32-1.20 (m, 3H), 1.18-1.10 (m, 2H), 0.89 (br s, 2H), 0.86-0.77 (m, 2H).

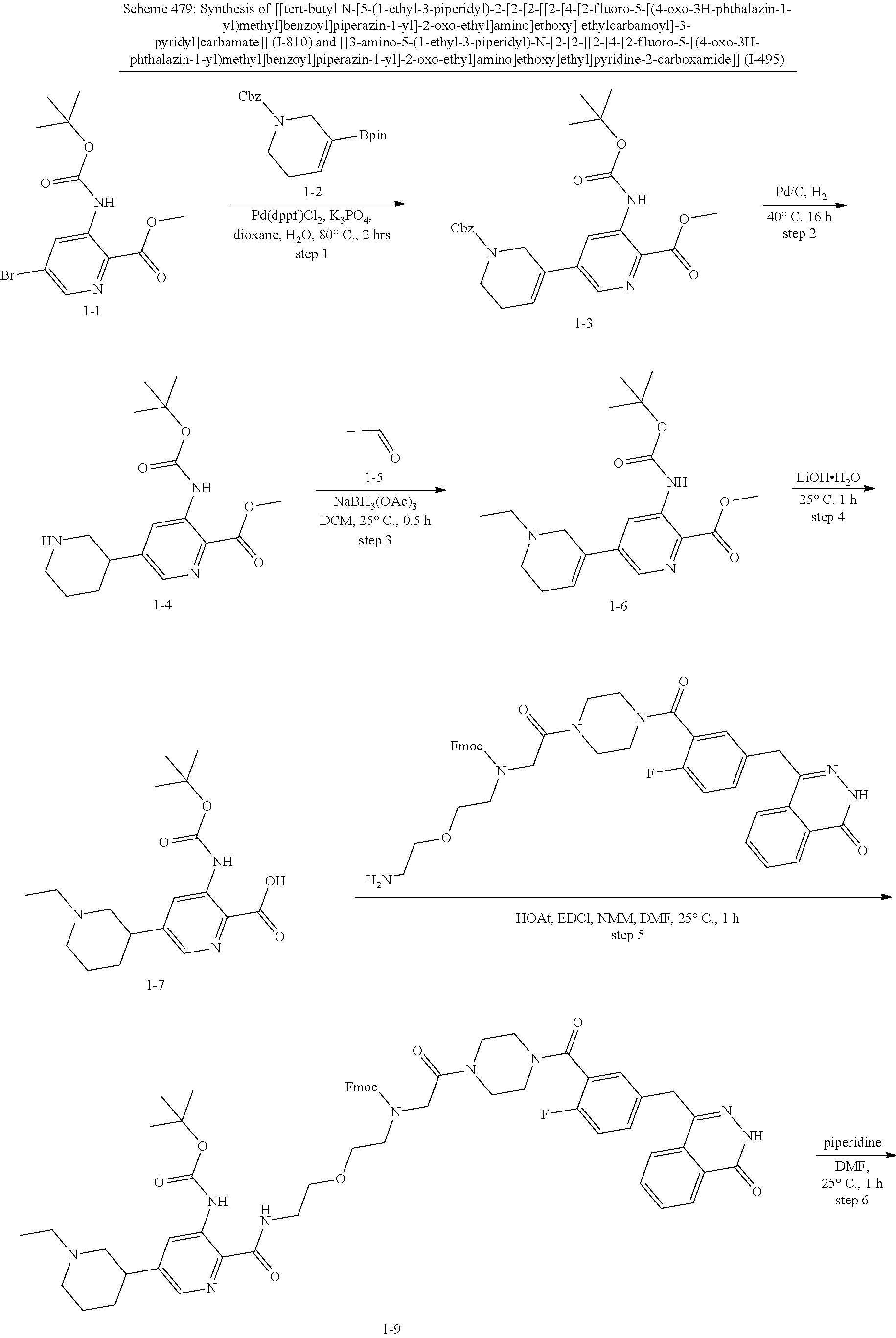

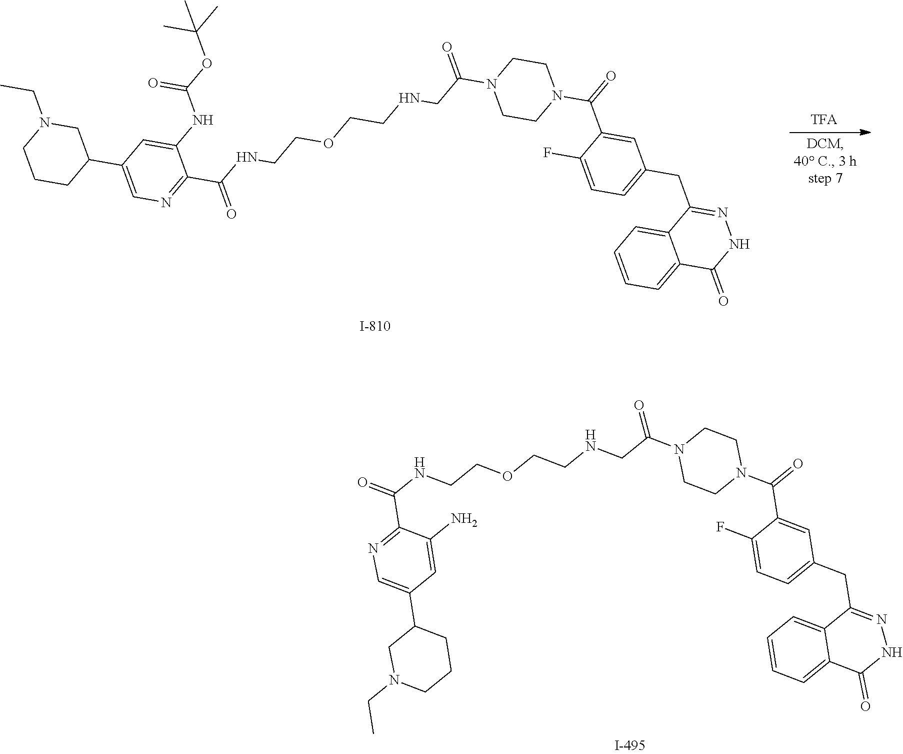 [SMILES: CCN1CCCC(c2cnc(C(=O)NCCOCCNCC(=O)N3CCN(C(=O)c4cc(Cc5n[nH]c(=O)c6ccccc56)ccc4F)CC3)c(NC(=O)OC(C)(C)C)c2)C1 ; CCN1CCCC(c2cnc(C(=O)NCCOCCNCC(=O)N3CCN(C(=O)c4cc(Cc5n[nH]c(=O)c6ccccc56)ccc4F)CC3)c(N)c2)C1]

### Step 1: Synthesis of Intermediate 1-3

To a solution of Intermediate 1-1 (1 g, 3.02 mmol, 1 eq) and Intermediate 1-2 (1.04 g, 3.02 mmol, 1 eq) in dioxane (10 mL) and H₂O (2 mL) was added Pd(dtbpf)Cl₂ (196.81 mg, 301.97 μmol, 0.1 eq) and K₃PO₄ (1.92 g, 9.06 mmol, 3 eq). The mixture was stirred at 80° C. for 2 hr. The reaction mixture was diluted with H₂O (10 mL) and extracted with DCM (10 mL*3). The combined organic layers were dried over Na₂SO₄, filtered and concentrated under reduced pressure to give a residue. The residue was purified by flash silica gel chromatography (ISCO; 40 g SepaFlash Silica Flash Column, Eluent of 0˜68% Ethyl acetate/Petroleum ether gradient @50 mL/min) and concentrated under reduced pressure to give Intermediate 1-3 (1 g) as a yellow solid. LCMS (Method D): Retention time: 0.473 min, [M+H]⁺=468.2.

### Step 2: Synthesis of Intermediate 1-4

To a solution of Intermediate 1-3 (900 mg, 1.93 mmol, 1 eq) in ethyl acetate (0.9 mL) was added Pd(OH)₂/C (20% w.t.%, 450 mg) under N₂ atmosphere. The suspension was degassed and purged with H₂ 3 times. The mixture was stirred under H₂ (15 Psi) at 40° C. for 16 hr. The reaction mixture was filtered and concentrated under reduced pressure to give a residue. The crude product was used in the next step without further purification. Intermediate 1-4 (750 mg, crude) as a black solid. LCMS (Method D): Retention time: 0.297 min, [M+H]⁺=336.2.

### Step 3: Synthesis of Intermediate 1-6

To a mixture of Intermediate 1-4 (680 mg, 2.03 mmol, 1 eq) and Intermediate 1-5 (107.18 mg, 2.43 mmol, 136.53 μL, 1.2 eq) in DCM (7 mL) was added acetic acid (243.50 mg, 4.05 mmol, 232.12 μL, 2 eq) and NaBH(OAc)₃ (2.15 g, 10.14 mmol, 5 eq), then the mixture was stirred at 20° C. for 30 min. The reaction mixture was diluted with H₂O (10 mL), and extracted with EA (10 mL*3). The combined organic layers were concentrated under reduced pressure to give a residue. The residue was purified by flash silica gel chromatography (ISCO; 40 g SepaFlash Silica Flash Column, Eluent of 0˜30% Dichloromethane/Methanol @60 mL/min) and concentrated under reduced pressure to give a residue. Intermediate 1-6 (185 mg) was obtained as a black solid. LCMS (Method D): Retention time: 0.316 min, [M+H]⁺=364.1.

### Step 4: Synthesis of Intermediate 1-7

To a solution of Intermediate 1-6 (105 mg, 288.90 μmol, 1 eq) in THF (1 mL), MeOH (1 mL) and H₂O (0.5 mL) was added LiOH H₂O (60.62 mg, 1.44 mmol, 5 eq). The mixture was stirred at 25° C. for 1 hr. The reaction mixture was concentrated under reduced pressure and then diluted with H₂O (5 mL) and adjusted to pH=6 with HCl (1 M). Then the mixture was extracted with DCM (5 mL*3). The combined organic layers were dried over Na₂SO₄, filtered and concentrated under reduced pressure to give a residue. The crude product was used in the next step without further purification. Intermediate 1-7 (164 mg, crude) was obtained as a black solid. LCMS (Method D): Retention time: 0.251 min, [M+H]⁺=305.2.

### Step 5: Synthesis of Intermediate 1-9

To a solution of Intermediate 1-7 (97 mg, 277.60 μmol, 1 eq) and Intermediate 1-8 (213.55 mg, 277.60 μmol, 1 eq, HCl) in DMF (1 mL) was added EDCI (159.65 mg, 832.80 μmol, 3 eq), NMM (140.39 mg, 1.39 mmol, 152.60 μL, 5 eq) and HOAt (37.78 mg, 277.60 μmol, 38.83 μL, 1 eq). The mixture was stirred at 25° C. for 1 hr. The reaction mixture was concentrated under reduced pressure to give a residue. The residue was purified by flash silica gel chromatography (ISCO; 20 g SepaFlash Silica Flash Column, Eluent of 0-40% Ethyl acetate/MeOH @36 mL/min) and concentrated under reduced pressure to give a residue. Intermediate 1-9 (300 mg) was obtained as a yellow oil. LCMS (Method D): Retention time: 0.416 min, [M+H]⁺=1064.3.

### Step 6: Synthesis of I-810

To a solution of Intermediate 1-9 (250 mg, 234.92 μmol, 1 eq) in DMF (2.5 mL) was added piperidine (40.00 mg, 469.83 μmol, 46.40 μL, 2 eq). The mixture was stirred at 25° C. for 1 h. The reaction mixture was filtered and concentrated under reduced pressure to give a residue. The crude product was purified by reversed-phase chromatography (neutral condition) followed by lyophilization to give product. I-810 (122.33 mg, 143.52 μmol, 61.09% yield, 98.780% purity) was obtained as a white solid. LCMS (Method D): Retention time: 0.317 min, [M+H]⁺=842.3. SFC: Retention time: 2.055 min, 2.567 min. ¹H NMR (400 MHz, METHANOL-d₄) δ=8.71-8.62 (m, 1H), 8.37 (d, J=7.2 Hz, 1H), 8.18-8.09 (m, 1H), 7.95 (d, J=8.0 Hz, 1H), 7.91-7.79 (m, 2H), 7.49 (d, J=3.6 Hz, 1H), 7.36 (d, J=5.2 Hz, 1H), 7.21-7.10 (m, 1H), 4.41-4.35 (m, 2H), 3.73 (s, 2H), 3.63 (d, J=2.8 Hz, 5H), 3.58-3.46 (m, 6H), 3.37 (s, 1H), 3.09-3.00 (m, 2H), 2.95-2.87 (m, 1H), 2.80 (s, 2H), 2.56-2.43 (m, 2H), 2.35-2.23 (m, 1H), 2.12-2.00 (m, 2H), 1.98-1.91 (m, 1H), 1.88-1.67 (m, 3H), 1.52 (s, 9H), 1.35-1.23 (m, 1H), 1.17-1.08 (m, 3H). ¹⁹F NMR (377 MHz, METHANOL-d₄), δ=−120.642.

### Step 7: Synthesis of I-495

To a solution of I-810 (72 mg, 85.51 μmol, 1 eq) in DCM (0.7 mL) was added TFA (9.75 mg, 85.51 μmol, 6.35 μL, 1 eq). The mixture was stirred at 40° C. for 3 hr. The reaction mixture was concentrated under reduced pressure to give a residue. The crude product was purified by reversed phase chromatography (0.1% TFA condition) followed by lyophilization to give product. I-495 (19.22 mg, 22.10 μmol, 25.84% yield, 98.406% purity, TFA salt) was obtained as a yellow solid. LCMS (Method H): Retention time: 0.494 min, [M+H]⁺=742.4. SFC: Retention time: 1.148 min, 1.441 min. ¹H NMR (400 MHz, METHANOL-d₄) δ =8.37 (d, J=7.6 Hz, 1H), 7.99-7.93 (m, 1H), 7.90-7.82 (m, 2H), 7.79 (d, J=10.8 Hz, 1H), 7.53-7.51 (m, 1H), 7.35 (t, J=5.6 Hz, 1H), 7.18 (t, J=9.2 Hz, 1H), 7.06 (d, J=3.2 Hz, 1H), 4.39 (s, 2H), 4.18-4.07 (m, 2H), 3.81 (d, J=3.2 Hz, 2H), 3.77-3.64 (m, 6H), 3.64-3.56 (m, 4H), 3.53 (d, J=4.8 Hz, 1H), 3.49-3.40 (m, 2H), 3.40-3.35 (m, 1H), 3.28-3.25 (m, 1H), 3.24-3.16 (m, 2H), 3.14-3.01 (m, 2H), 3.01-2.87 (m, 2H), 2.18-2.08 (m, 1H), 2.07-1.99 (m, 1H), 1.98-1.85 (m, 1H), 1.82-1.69 (m, 1H), 1.39-1.29 (m, 3H). ¹⁹F NMR (377 MHz, METHANOL-d₄), δ=−77.084, −120.635.

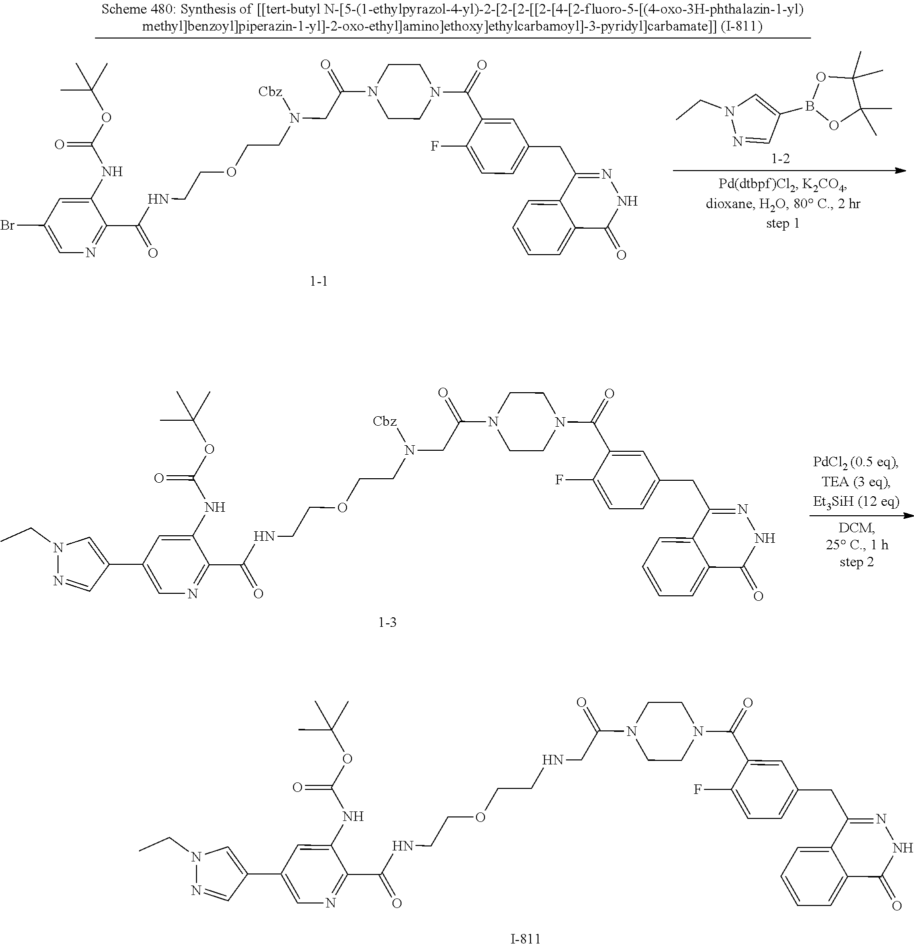 [SMILES: CC(C)(C)OC(=O)Nc1cc(Br)cnc1C(=O)NCCOCCN(CC(=O)N1CCN(C(=O)c2cc(Cc3n[nH]c(=O)c4ccccc34)ccc2F)CC1)C(=O)OCc1ccccc1 ; CCn1cc(B2OC(C)(C)C(C)(C)O2)cn1 ; CCn1cc(-c2cnc(C(=O)NCCOCCN(CC(=O)N3CCN(C(=O)c4cc(Cc5n[nH]c(=O)c6ccccc56)ccc4F)CC3)C(=O)OCc3ccccc3)c(NC(=O)OC(C)(C)C)c2)cn1 ; CCn1cc(-c2cnc(C(=O)NCCOCCNCC(=O)N3CCN(C(=O)c4cc(Cc5n[nH]c(=O)c6ccccc56)ccc4F)CC3)c(NC(=O)OC(C)(C)C)c2)cn1]

### Step 1: Synthesis of Intermediate 1-3

To a solution of intermediate 1-1 (200 mg, 202.05 gmol, 1 eq, FA salt) and intermediate 1-2 (44.87 mg, 202.05 μmol, 1 eq) in H₂O (0.3 mL) and dioxane (1.5 mL) was added K₃PO₄ (128.67 mg, 606.16 μmol, 3 eq) and Pd(dtbpf)Cl₂ (13.17 mg, 20.21 μmol, 0.1 eq). The mixture was stirred at 80° C. for 2 hr. The reaction mixture was filtered and concentrated under reduced pressure to give a residue. The residue was purified by flash silica gel chromatography (ISCO; 20 g SepaFlash Silica Flash Column, Eluent of 0˜70% Ethyl acetate/Methanol @50 mL/min). Intermediate 1-3 (230 mg, 96% purity) was obtained as a white solid. LCMS (Method D): Rt=0.547 min [M+H]⁺=959.8.

### Step 2: Synthesis of I-811

To a solution of intermediate 1-3 (80 mg, 83.42 μmol, 1 eq) in DCM (0.9 mL) was added PdCl₂ (7.40 mg, 41.71 μmol, 0.5 eq), TEA (25.32 mg, 250.25 gmol, 34.83 μL, 3 eq) and Et₃SiH (116.40 mg, 1.00 mmol, 159.89 μL, 12 eq) dropwise. The mixture was stirred at 25° C. for 1 hr. The reaction mixture was diluted with NaHCO₃ (5 mL), filtered and extracted with DCM (5 mL*3). The combined organic layers were dried over Na₂SO₄, filtered and concentrated under reduced pressure to give a residue. The crude product was purified by reversed-phase chromatography (0.1% FA condition) followed by lyophilization to give product. I-811 (22.09 mg, 25.36 gmol, 30.41% yield, 100% purity, FA salt) was obtained as a white solid. LCMS (Method F): Rt=0.632 min [M+H]⁺=825.5. ¹H NMR (400 MHz, METHANOL-d4) δ=8.88 (s, 1H), 8.53 (s, 1H), 8.44 (d, J=9.2 Hz, 1H), 8.36 (d, J=7.2 Hz, 1H), 8.19 (s, 1H), 7.98-7.90 (m, 2H), 7.89-7.79 (m, 2H), 7.46-7.48 (m, 1H), 7.32-7.34 (m, 1H), 7.19-7.10 (m, 1H), 4.37 (d, J=5.6 Hz, 2H), 4.31-4.16 (m, 2H), 3.81-3.53 (m, 12H), 3.49 (d, J=4.0 Hz, 2H), 3.34 (s, 1H), 3.27 (s, 1H), 2.97-2.88 (m, 2H), 1.56-1.45 (m, 12H). ¹⁹F NMR (377 MHz, METHANOL-d₄), δ=120.650.

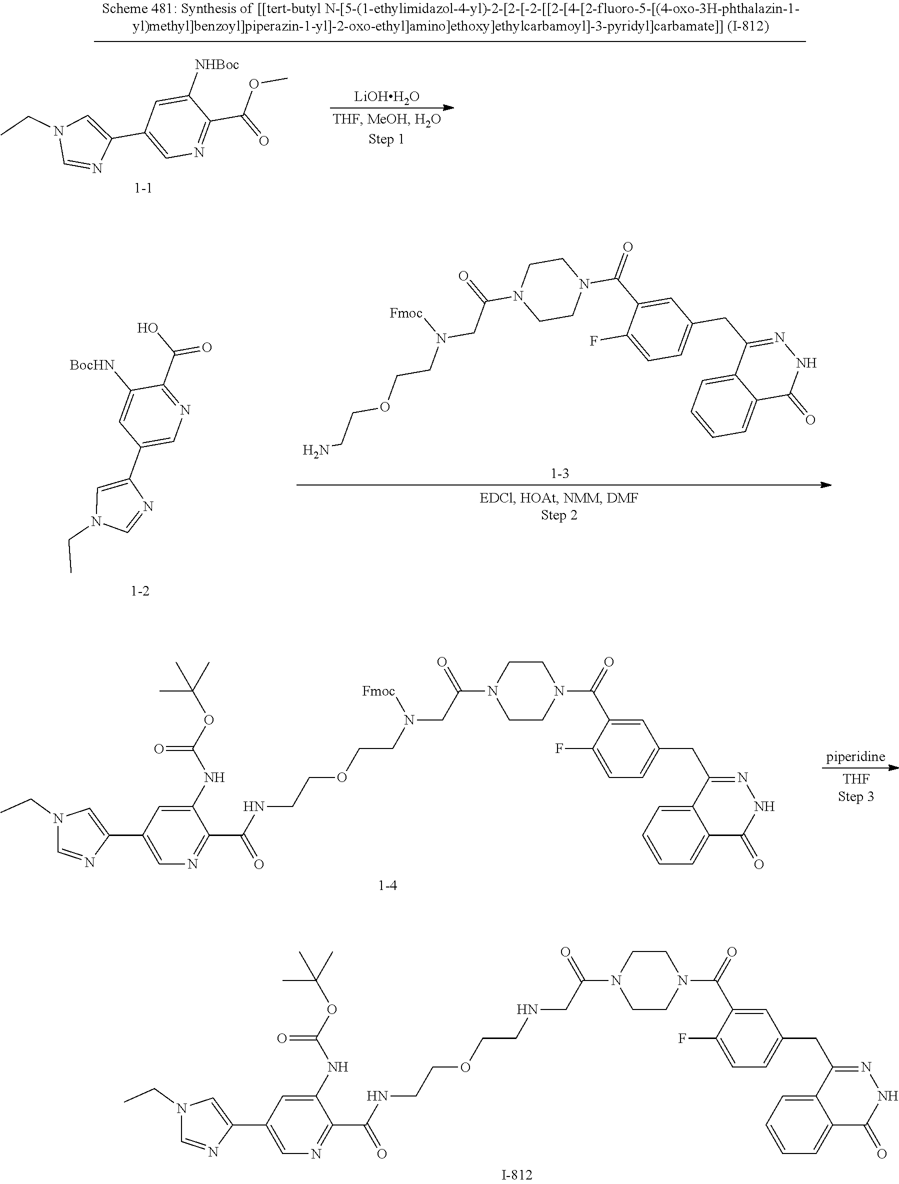

### Step 1: Synthesis of Intermediate 1-2

To a solution of intermediate 1-1 (180 mg, 519.66 μmol, 1 eq) in MeOH (0.6 mL), THF (0.6 mL) and H₂O (0.6 mL) was added LiOH H₂O (43.61 mg, 1.04 mmol, 2 eq). The mixture was stirred at 25° C. for 1 hr. The residue was diluted with H₂O (5 mL) and extracted with DCM (5 mL*3), dried over Na₂SO₄, filtered and concentrated under reduced pressure to give a residue. The crude product was used in the next step without further purification. Intermediate 1-2 (120 mg, 361.06 μmol, 69.48% yield) was obtained as a brown solid. LCMS (Method D): Rt=0.275 min, [M+H]⁺=333.1.

### Step 2: Synthesis of Intermediate 1-4

To a solution of intermediate 1-2 (100 mg, 300.88 μmol, 1.5 eq) in DMF (1 mL) was added HOAt (27.30 mg, 200.59 μmol, 28.06 μL, 1 eq), EDCI (115.36 mg, 601.77 μmol, 3 eq), NMM (101.45 mg, 1.00 mmol, 110.27 μL, 5 eq) and intermediate 1-3 (154.31 mg, 200.59 μmol, 1 eq, HCl salt). The mixture was stirred at 25° C. for 1 hr. The reaction liquid was concentrated under vacuum to afford the crude product. The crude product was purified by reversed phase chromatography (0.1% NH₃·H₂O), which was concentrated and lyophilized to afford intermediate 1-4 (20 mg, 19.10 μmol, 9.52% yield) as a yellow solid. LCMS: Rt=2.023 min, [M+H]⁺=1047.4 Step 3: Synthesis of I-812

To a solution of intermediate 1-4 (15 mg, 14.32 μmol, 1 eq) in THF (0.15 mL) was added piperidine (16.17 mg, 189.86 μmol, 0.01875 mL, 13.25 eq). The mixture was stirred at 25° C. for 1 hr. The reaction liquid was concentrated under vacuum to afford the crude product. The crude product was purified by reversed phase chromatography (0.1% NH₃·H₂O condition), then the eluent was concentrated and lyophilized to afford I-812 (6.23 mg, 7.55 μmol, 52.72% yield, 100.00% purity) as a white solid. LCMS (Method D): Rt=0.342 min, [M+H]⁺=825.5. ¹H NMR (400 MHz, METHANOL-d4) δ=9.08-9.01 (m, 1H), 8.59 (d, J=6.4 Hz, 1H), 8.36 (d, J=7.6 Hz, 1H), 7.96-7.90 (m, 1H), 7.90-7.79 (m, 2H), 7.79-7.73 (m, 2H), 7.51-7.43 (m, 1H), 7.34 (d, J=6.0 Hz, 1H), 7.18-7.10 (m, 1H), 4.37 (d, J=6.4 Hz, 2H), 4.17-4.07 (m, 2H), 3.77-3.68 (m, 2H), 3.68-3.63 (m, 4H), 3.60-3.55 (m, 2H), 3.54-3.44 (m, 4H), 3.34 (s, 2H), 3.26 (s, 2H), 2.81 (d, J=3.6 Hz, 2H), 1.53 (s, 9H), 1.52-1.44 (m, 3H). ¹⁹F NMR (377 MHz, METHANOL-d₄), δ=−120.712.

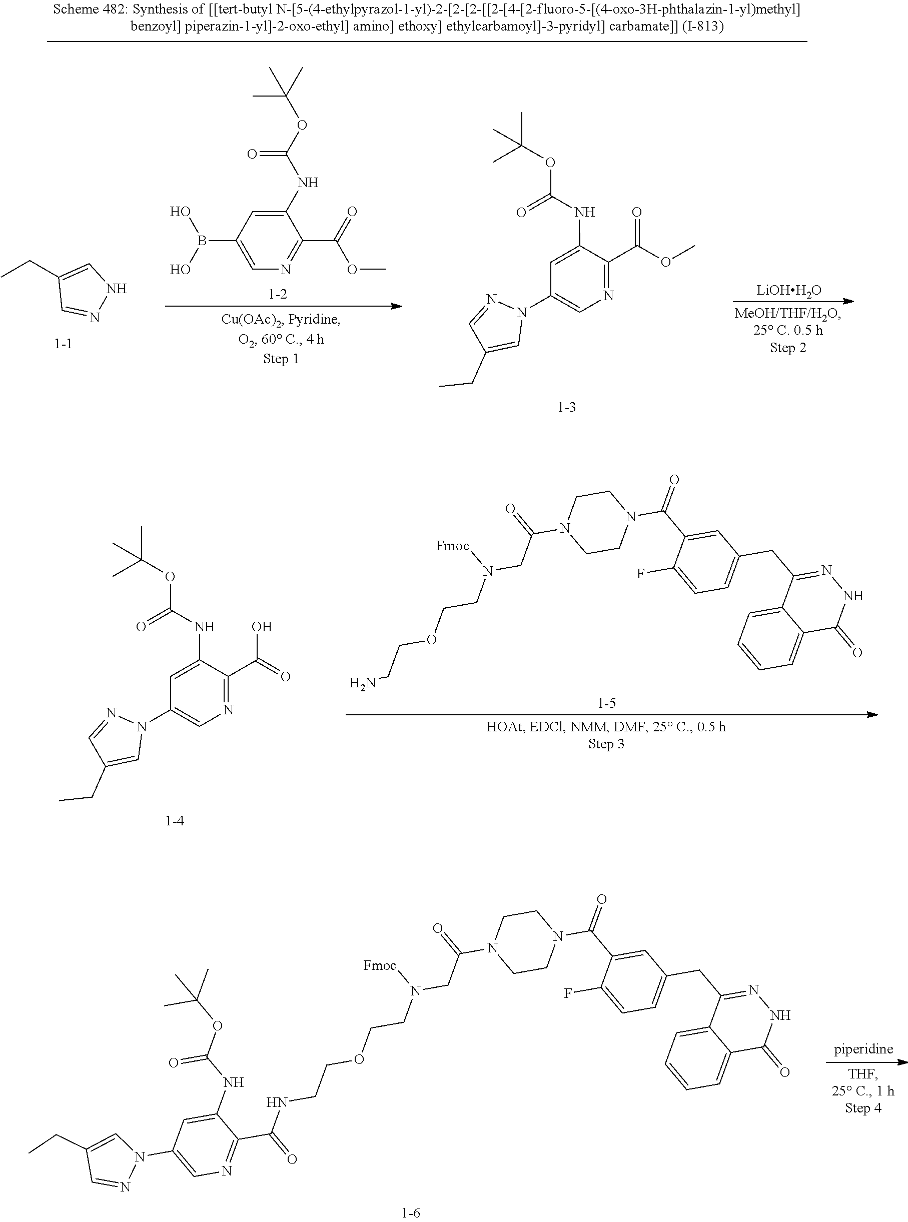

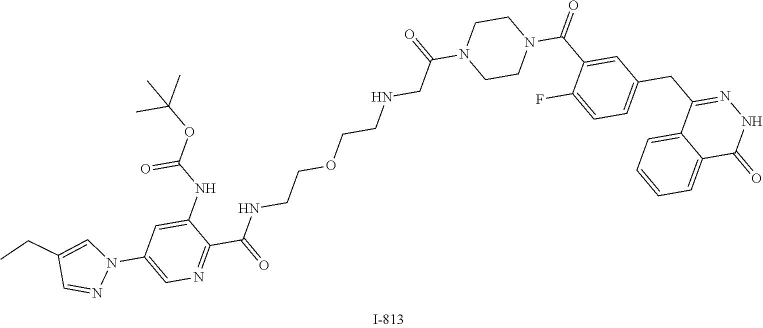 [SMILES: CCc1cnn(-c2cnc(C(=O)NCCOCCNCC(=O)N3CCN(C(=O)c4cc(Cc5n[nH]c(=O)c6ccccc56)ccc4F)CC3)c(NC(=O)OC(C)(C)C)c2)c1]

### Step 1: Synthesis of Intermediate 1-3

To a solution of intermediate 1-1 (64.93 mg, 675.49 μmol, 1 eq) and intermediate 1-2 (300.00 mg, 1.01 mmol, 1.5 eq) in pyridine (3 mL) was added Cu(OAc)₂ (245.38 mg, 1.35 mmol, 2 eq). The mixture was stirred at 60° C. for 4 hr. The reaction mixture was concentrated under reduced pressure to give a residue. The residue was purified by flash silica gel chromatography (ISCO; 12 g SepaFlashSilica Flash Column, Eluent of 0˜100% PE:EA @36 mL/min, PE:EA=10:1, Rf=0.5) and the eluent was concentrated under reduced pressure to give a product. Intermediate 1-3 (70 mg, 202.09 μmol, 29.92% yield) was obtained as a yellow gum. LCMS (Method D): Rt=0.493 min, [M+H]⁺=347.1.

### Step 2: Synthesis of Intermediate 1-4

To a solution of intermediate 1-3 (60 mg, 173.22 μmol, 1 eq) in MeOH (0.2 mL), THF (0.2 mL) and H₂O (0.2 mL) was added LiOH H₂O (21.81 mg, 519.66 μmol, 3 eq). The mixture was stirred at 25° C. for 0.5 hr. The reaction mixture was poured into H₂O (10 mL) at 25° C., the pH was adjusted to 3 with 1M aqueous HCl, and then the mixture was extracted with DCM (5 mL*3). The combined organic layers were dried over Na₂SO₄, filtered and concentrated under reduced pressure to give a product. Intermediate 1-4 (30 mg, crude) was obtained as a white gum. LCMS (Method D): Rt=0.461 min, [M+H]⁺=333.1.

### Step 3: Synthesis of Intermediate 1-6

To a solution of intermediate 1-4 (30 mg, 90.27 μmol, 1 eq) and intermediate 1-5 (69.44 mg, 90.27 μmol, 1 eq, HCl salt) in DMF (0.5 mL) was added EDCI (51.91 mg, 270.80 μmol, 3 eq), HOAt (12.29 mg, 90.27 μmol, 12.63 μL, 1 eq) and NMM (45.65 mg, 451.33 μmol, 49.62 μL, 5 eq). The mixture was stirred at 25° C. for 0.5 hr. The reaction mixture was poured into H₂O (2 mL), and extracted with EA (2 mL*3). The combined organic layers were dried over Na₂SO₄, filtered and concentrated under reduced pressure to give a residue. The residue was purified by flash silica gel chromatography (ISCO; 4 g Sepa FlashSilica Flash Column, Eluent of 0˜100% DCM/MeOH@18 mL/min, DCM/MeOH=10:1, Rf=0.2) and the eluent was concentrated under reduced pressure to give a product. Intermediate 1-6 (65 mg, 62.07 μmol, 68.77% yield) was obtained as a yellow gum. LCMS (Method D): Rt=0.533 min, [M+H]⁺=1047.3.

### Step 4: Synthesis of I-813

To a solution of intermediate 1-6 (55 mg, 52.52 μmol, 1 eq) in THF (0.6 mL) was added piperidine (4.47 mg, 52.52 μmol, 5.19 μL, 1 eq). The mixture was stirred at 25° C. for 1 hr. The reaction mixture was concentrated under reduced pressure to give a residue. The residue was purified by prep-HPLC (column: CD01-Phenomenex luna C18 150*25*10 um; mobile phase: [water (FA)-ACN]; gradient: 26%-566% B over 10 min) and the eluent was concentrated to remove MeCN and then lyophilized. I-813 (14.9 mg, 18.06 μmol, 34.39% yield, 100% purity) was obtained as a white solid. LCMS (Method D): Rt=0.399 min, (M+H)+=825.3. ¹H NMR (400 MHz, METHANOL-d4) δ=9.06 (s, 1H), 8.62 (s, 1H), 8.35 (d, J=7.6 Hz, 1H), 8.11 (s, 1H), 7.91 (s, 1H), 7.87-7.76 (m, 2H), 7.62 (d, J=15.6 Hz, 1H), 7.47 (s, 1H), 7.35 (d, J=2.8 Hz, 1H), 7.14 (d, J=7.6 Hz, 1H), 4.36 (s, 2H), 3.71 (d, J=10.8 Hz, 2H), 3.68-3.56 (m, 7H), 3.55-3.44 (m, 4H), 3.35 (s, 1H), 3.25 (s, 2H), 2.81 (s, 2H), 2.65-2.48 (m, 2H), 1.54 (s, 9H), 1.31-1.22 (m, 3H). ¹⁹F NMR (400 MHz, METHANOL-d4) δ=−120.659.

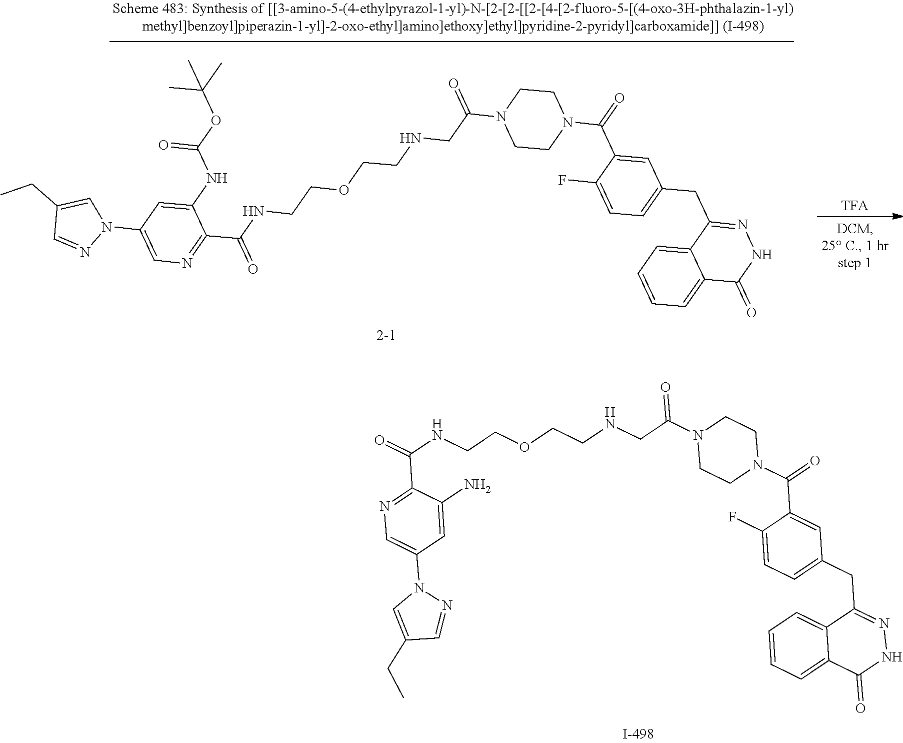 [SMILES: CCc1cnn(-c2cnc(C(=O)NCCOCCNCC(=O)N3CCN(C(=O)c4cc(Cc5n[nH]c(=O)c6ccccc56)ccc4F)CC3)c(NC(=O)OC(C)(C)C)c2)c1 ; CCc1cnn(-c2cnc(C(=O)NCCOCCNCC(=O)N3CCN(C(=O)c4cc(Cc5n[nH]c(=O)c6ccccc56)ccc4F)CC3)c(N)c2)c1]

To a solution of intermediate 2-1 (40 mg, 48.49 μmol, 1 eq) in DCM (0.3 mL) was added TFA (0.15 mL). The mixture was stirred at 25° C. for 1 hr. The residue was diluted with H₂O (5 mL) and extracted with EA (5 mL*3), dried over Na₂SO₄, filtered and concentrated under reduced pressure to give a residue. The crude product was purified by reversed phase chromatography (0.1% TFA condition). The eluent was concentrated under reduced pressure to remove ACN, then lyophilized to afford product. I-498 (7 mg, 8.15 μmol, 16.81% yield, 97.672% purity, TFA salt) was obtained as a white solid. L CMS (Method D). Rt=0.352 min, [M+H]⁺=725.2. ¹H NMR (400 MHz, METHANOL-d4) δ=8.37 (d, J=8.0 Hz, 1H), 8.24 (d, J=11.6 Hz, 1H), 8.06 (s, 1H), 7.97-7.91 (m, 1H), 7.91-7.79 (m, 2H), 7.61 (d, J=6.4 Hz, 1H), 7.55-7.46 (m, 1H), 7.41 (br d, J=9.2 Hz, 1H), 7.37-7.31 (m, 1H), 7.21-7.10 (m, 1H), 4.38 (d, J=2.4 Hz, 2H), 4.19-3.98 (m, 2H), 3.80 (d, J=2.4 Hz, 2H), 3.77-3.63 (m, 5H), 3.63-3.56 (m, 2H), 3.54-3.43 (m, 3H), 3.15 (d, J=11.6 Hz, 4H), 2.66-2.47 (m, 2H), 1.32-1.16 (m, 3H). 19F NMR (377 MHz, METHANOL-d4) δ=−120.658.

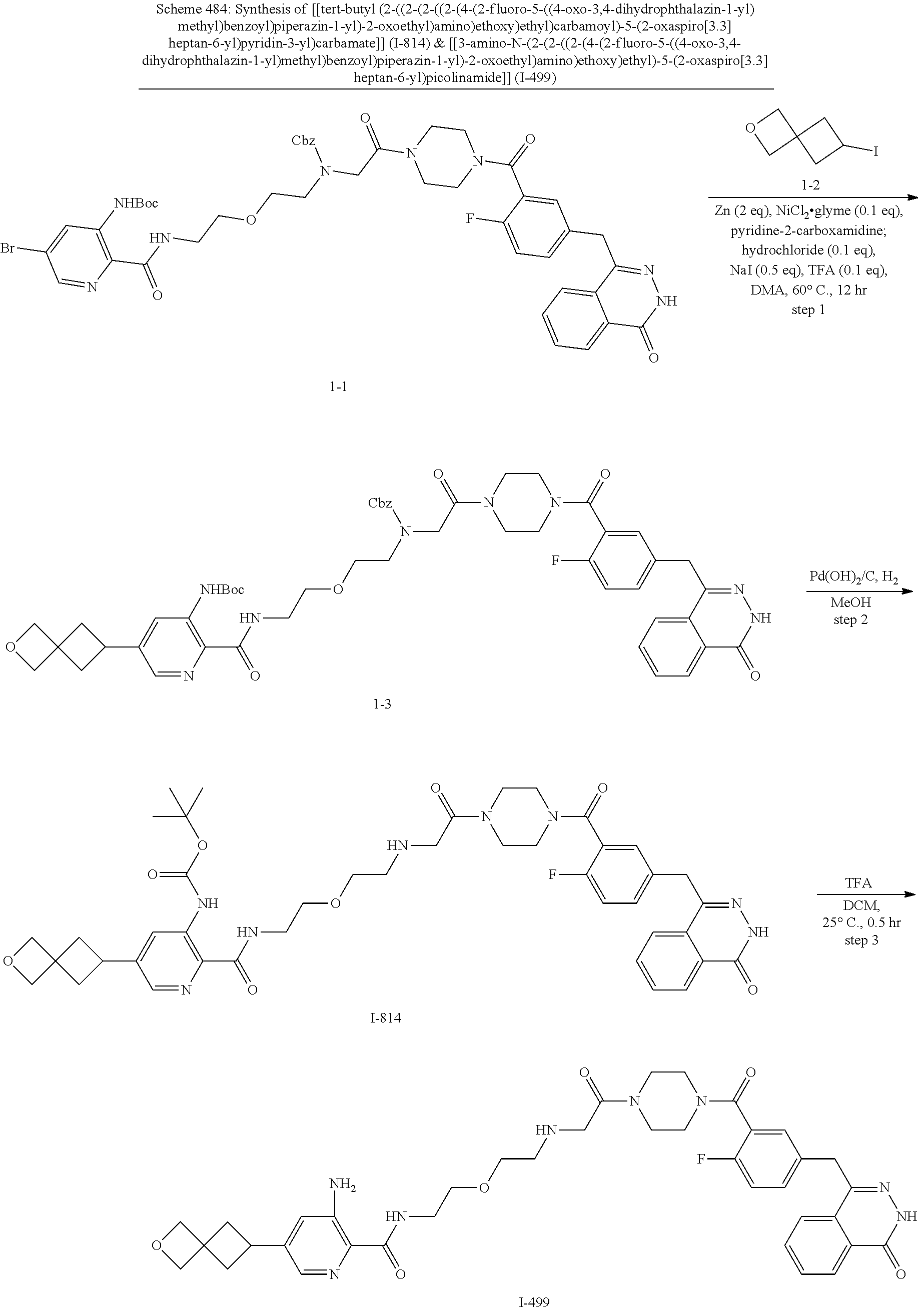 [SMILES: CC(C)(C)OC(=O)Nc1cc(Br)cnc1C(=O)NCCOCCN(CC(=O)N1CCN(C(=O)c2cc(Cc3n[nH]c(=O)c4ccccc34)ccc2F)CC1)C(=O)OCc1ccccc1 ; IC1CC2(COC2)C1 ; CC(C)(C)OC(=O)Nc1cc(C2CC3(COC3)C2)cnc1C(=O)NCCOCCN(CC(=O)N1CCN(C(=O)c2cc(Cc3n[nH]c(=O)c4ccccc34)ccc2F)CC1)C(=O)OCc1ccccc1 ; CC(C)(C)OC(=O)Nc1cc(C2CC3(COC3)C2)cnc1C(=O)NCCOCCNCC(=O)N1CCN(C(=O)c2cc(Cc3n[nH]c(=O)c4ccccc34)ccc2F)CC1 ; Nc1cc(C2CC3(COC3)C2)cnc1C(=O)NCCOCCNCC(=O)N1CCN(C(=O)c2cc(Cc3n[nH]c(=O)c4ccccc34)ccc2F)CC1]

### Step 1: Synthesis of Intermediate 1-3

A mixture of Intermediate 1-1 (500 mg, 505.13 μmol, 1 eq, FA salt), Intermediate 1-2 (226.34 mg, 1.01 mmol, 2 eq), NiCl₂-glyme (110.99 mg, 505.13 μmol, 1 eq), NaI (7.57 mg, 50.51 μmol, 0.1 eq), pyridine-2-carboxamidine; hydrochloride (7.96 mg, 50.51 μmol, 0.1 eq) and TFA (28.80 mg, 252.57 μmol, 18.76 μL, 0.5 eq) in DMA (5 mL) was degassed and purged with N₂ 3 times, and then Zn (66.06 mg, 1.01 mmol, 2 eq) was added into the system, and the mixture was stirred at 60° C. for 12 hr under N₂ atmosphere. The reaction mixture was filtered and diluted with H₂O (10 mL) and then extracted with EA (5 mL*3). The combined organic layers were dried over sodium sulfate, filtered and concentrated under reduced pressure to give a residue. The residue was purified by prep-HPLC (column: CD08-Phenomenex kinetex EVO C18 150*30*5 um; mobile phase: [water (FA)-ACN]; B %:%, isocratic elution mode), the eluent was concentrated and lyophilized to give the desired product. Intermediate 1-3 (250 mg, 248.25 μmol, 49.14% yield, 100% purity, FA salt) was obtained as a white solid. LCMS (Method D): Retention time: 0.503 min, [M+H]⁺=961.3. ¹H NMR (400 MHz, METHANOL-d4) δ=8.80-8.52 (m, 1H), 8.36 (d, J=7.6 Hz, 1H), 8.23-7.91 (m, 2H), 7.90-7.78 (m, 2H), 7.54-7.46 (m, 1H), 7.41-7.23 (m, 6H), 7.21-7.12 (m, 1H), 5.12-4.98 (m, 2H), 4.65-4.58 (m, 2H), 4.39 (s, 2H), 4.22 (d, J=17.2 Hz, 2H), 3.80-3.38 (m, 16H), 3.15-3.01 (m, 1H), 2.76-2.59 (m, 1H), 2.42-2.24 (m, 2H), 1.51 (s, 10H).

### Step 2: Synthesis of I-814

To a solution of Pd(OH)₂/C (30 mg, 42.72 μmol, 20% purity) in MeOH (0.5 mL) was added Intermediate 1-3 (45 mg, 44.68 μmol, 1 eq, FA salt). The mixture was stirred at 25° C. for 1 hr under H₂ (15 Psi), after degassing and backfilling with H₂ three times. The reaction mixture was filtered and washed with MeOH (3 mL*2), The filtrate was concentrated under reduced pressure to give a product. The crude product was used for the next step without further purification. I-814 (38 mg, 42.91 μmol, 96.02% yield, 98.563% purity, FA salt) was obtained as a white solid. LCMS (Method D): Retention time: 0.394 min, [M+H]⁺=827.2. ¹H NMR (400 MHz, METHANOL-d4) δ=8.62 (s, 1H), 8.55 (s, 1H), 8.37 (d, J=8.0 Hz, 1H), 8.07-8.01 (m, 1H), 7.98-7.92 (m, 1H), 7.89-7.79 (m, 2H), 7.48 (d, J=5.2 Hz, 1H), 7.38-7.31 (m, 1H), 7.20-7.13 (m, 1H), 4.64 (d, J=3.2 Hz, 2H), 4.38 (s, 2H), 3.76-3.70 (m, 2H), 3.67-3.60 (m, 6H), 3.58-3.52 (m, 4H), 3.49-3.45 (m, 2H), 3.44-3.39 (m, 1H), 3.39-3.33 (m, 2H), 2.83-2.67 (m, 4H), 2.41-2.31 (m, 2H), 1.52 (s, 11H). ¹⁹F NMR (376 MHz, METHANOL-d4) δ=−76.95, −120.70.

### Step 3: Synthesis of I-499

To a solution of I-814 (20 mg, 22.91 μmol, 1 eq, FA salt) in DCM (0.2 mL) was added TFA (0.04 mL). The mixture was stirred at 25° C. for 0.5 hr. The mixture was diluted with H₂O (2 mL) and adjusted to pH 9 with NH₃·H₂O, then the mixture was extracted with EA (5 mL*3), the organic layers were dried over Na₂SO₄, filtered and concentrated to give a residue. The crude product was purified by reversed phase chromatography (neutral condition), and the eluent was concentrated and lyophilized to give the desired product. I-499 (6.30 mg, 8.58 μmol, 37.45% yield, 98.976% purity) was obtained as a white solid. LCMS (Method D): Retention time: 0.319 min, [M+H]⁺=727.3. ¹H NMR (400 MHz, METHANOL-d4) δ=8.37 (d, J=7.6 Hz, 1H), 7.97-7.93 (m, 1H), 7.91-7.79 (m, 2H), 7.73-7.63 (m, 1H), 7.49 (s, 1H), 7.36 (d, J=4.0 Hz, 1H), 7.21-7.11 (m, 1H), 7.06-6.90 (m, 1H), 4.60 (s, 2H), 4.38 (s, 2H), 3.79-3.69 (m, 3H), 3.66 (s, 7H), 3.60-3.45 (m, 6H), 3.39-3.32 (m, 3H), 2.90 (s, 2H), 2.71-2.61 (m, 2H), 2.34-2.25 (m, 2H). ¹⁹F NMR (376 MHz, METHANOL-d4) δ=−120.69.

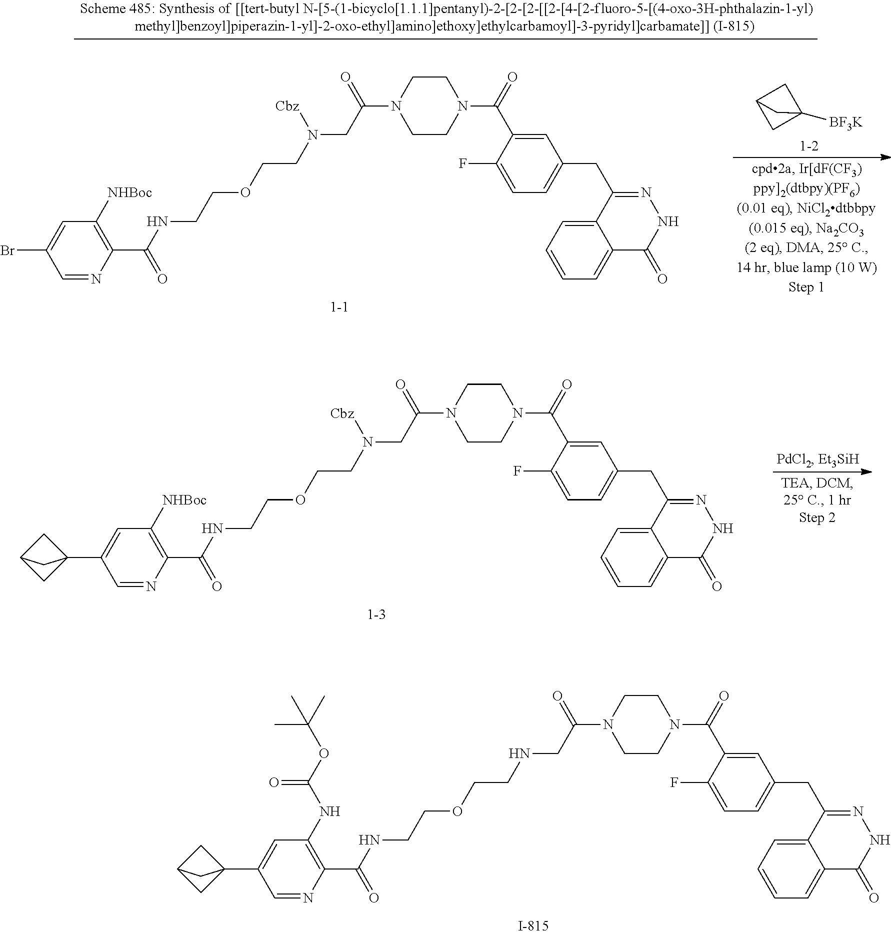

### Step 1: Synthesis of Intermediate 1-2

To a solution of Intermediate 1-2 (276.56 mg, 1.59 mmol, 1.5 eq) and Intermediate 1-1 (1 g, 1.06 mmol, 1 eq) in DMA (10 mL) was added Ir[dF(CF₃)ppy]₂(bpy)(PF₆) (106.98 mg, 105.95 μmol, 0.1 eq), Na₂CO₃ (224.60 mg, 2.12 mmol, 2 eq) and NiCl₂dtbbpy (63.25 mg, 158.93 μmol, 0.15 eq). The vial was sealed, degassed and backfilled with nitrogen. The reaction was stirred and irradiated with a blue 10 W LED lamp (3 cm away), with water cooling to keep the reaction temperature at 25° C. for 14 hr. The reaction mixture was diluted with water (40 mL) and extracted with ethyl acetate (40 mL*3). The combined organic layers were washed with brine (40 mL*3) and dried over sodium sulfate, filtered and concentrated under reduced pressure to give a residue. The crude product was purified by reversed-phase chromatography (H₂O). The eluent was concentrated under reduced pressure to remove ACN and then lyophilized to give Intermediate 1-3 (220 mg, 221.32 μmol, 20.89% yield, 93.662% purity) as a brown solid. LCMS (Method D): Rt: 0.548 min, [M+H]⁺=931.4. ¹H NMR (400 MHz, METHANOL-d4) δ=8.62-8.46 (m, 1H), 8.36 (d, J=7.6 Hz, 1H), 8.10-7.91 (m, 2H), 7.89-7.80 (m, 2H), 7.54-7.45 (m, 1H), 7.37-7.24 (m, 6H), 7.16 (t, J=9.2 Hz, 1H), 5.11 (s, 1H), 5.00 (s, 1H), 4.38 (s, 2H), 4.22 (d, J=17.6 Hz, 2H), 3.73 (s, 1H), 3.67-3.49 (m, 12H), 3.39 (s, 1H), 3.25-3.19 (m, 1H), 3.13-3.03 (m, 1H), 2.60-2.51 (m, 1H), 2.16 (d, J=12.4 Hz, 3H), 2.07 (d, J=8.8 Hz, 3H), 1.51 (d, J=4.4 Hz, 9H).

### Step 2: Synthesis of I-815

To a solution of Intermediate 1-2 (50 mg, 53.70 μmol, 1 eq) in DCM (1 mL) was added PdCl₂ (952.33 g, 5.37 μmol, 0.1 eq) and TEA (16.30 mg, 161.11 μmol, 22.43 μL, 3 eq). The suspension was degassed and purged with N₂ 3 times. Then Et₃SiH (31.22 mg, 268.52 μmol, 42.89 μL, 5 eq) was added into the mixture and it was stirred at 25° C. for 1 hr. The mixture was concentrated to give a residue. The crude product was purified by reversed phase chromatography (0.1% FA condition). The eluent was concentrated under reduced pressure to remove ACN and then lyophilized to give I-815 (10.39 mg, 11.77 μmol, 21.92% yield, 95.517% purity, FA salt) as a yellow solid. LCMS (Method D): Rt: 0.425 min, [M+H]⁺=797.3. ¹H NMR (400 MHz, METHANOL-d4) δ=8.58-8.52 (m, 1H), 8.36 (d, J=7.6 Hz, 1H), 8.06 (s, 1H), 7.97-7.91 (m, 1H), 7.90-7.78 (m, 2H), 7.49 (s, 1H), 7.37-7.26 (m, 2H), 7.19-7.13 (m, 1H), 4.38 (s, 2H), 3.94-3.41 (m, 15H), 3.04 (s, 2H), 2.58 (s, 1H), 2.42 (s, 1H), 2.16 (s, 6H), 1.52 (s, 9H). ¹⁹F NMR (400 MHz, METHAN OL-d4) δ=−120.637.

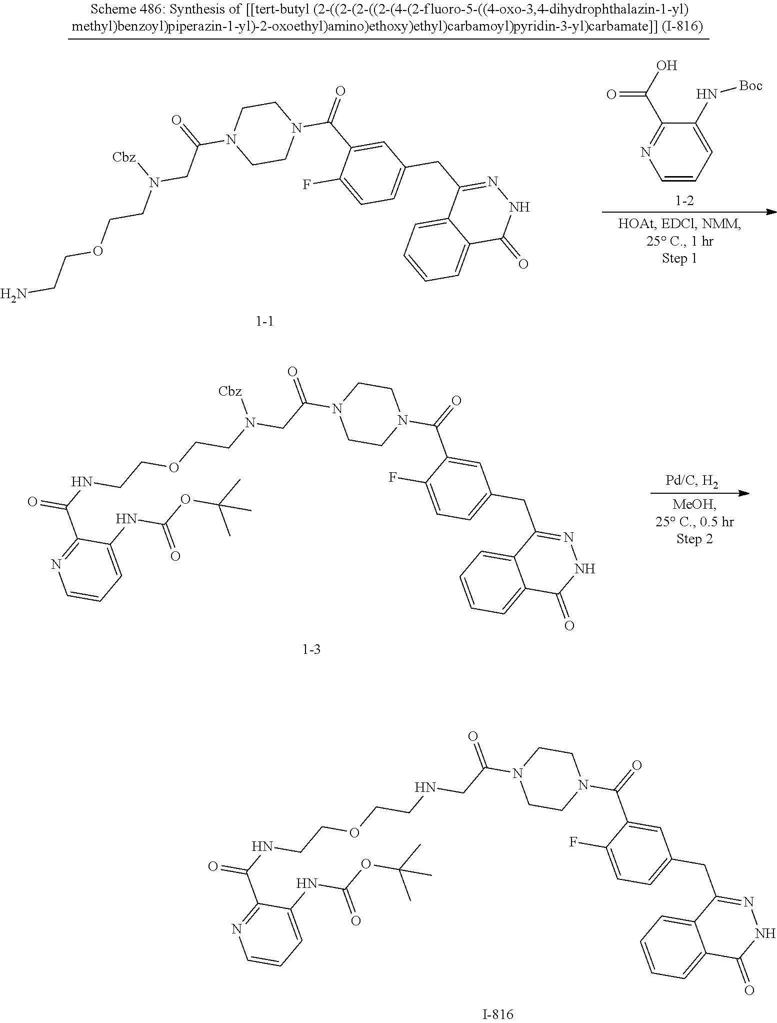 [SMILES: NCCOCCN(CC(=O)N1CCN(C(=O)c2cc(Cc3n[nH]c(=O)c4ccccc34)ccc2F)CC1)C(=O)OCc1ccccc1 ; CC(C)(C)OC(=O)Nc1cccnc1C(=O)O ; CC(C)(C)OC(=O)Nc1cccnc1C(=O)NCCOCCN(CC(=O)N1CCN(C(=O)c2cc(Cc3n[nH]c(=O)c4ccccc34)ccc2F)CC1)C(=O)OCc1ccccc1 ; CC(C)(C)OC(=O)Nc1cccnc1C(=O)NCCOCCNCC(=O)N1CCN(C(=O)c2cc(Cc3n[nH]c(=O)c4ccccc34)ccc2F)CC1]

### Step 1: Synthesis of Intermediate 1-3

To a solution of Intermediate 1-1 (0.5 g, 775.57 μmol, 1 eq) and Intermediate 1-2 (184.77 mg, 775.57 μmol, 1 eq) in DMF (5 mL) was added EDCI (446.03 mg, 2.33 mmol, 3 eq), HOAt (105.56 mg, 775.57 μmol, 108.49 μL, 1 eq), and NMM (392.23 mg, 3.88 mmol, 426.34 μL, 5 eq). The mixture was stirred at 25° C. for 0.5 hr. The reaction mixture was filtered and concentrated under reduced pressure to give a residue. The residue was purified by reversed phase chromatography (0.1% FA condition) and the eluent was concentrated to remove ACN and lyophilized to give Intermediate 1-3 (0.25 g, 289.05 μmol, 37.27% yield) as a white solid. LCMS (Method D): Retention time: 0.526 min, [M+H]⁺=865.4.

### Step 2: Synthesis of I-816

A mixture of Intermediate 1-3 (0.12 g, 138.74 μmol, 1 eq) and Pd/C (60 mg, 56.38 μmol, 10% purity, 4.06e−1 eq) in MeOH (1 mL) was degassed and purged with H₂ 3 times, and then the mixture was stirred at 25° C. for 0.5 hr under H₂ (15 psi) atmosphere. The reaction mixture was filtered and concentrated under reduced pressure to give a residue. The residue was purified by prep-HPLC (column: CD02-Waters Xbidge BEH C18 150*25*10 um; mobile phase: [water (NH₃H₂O)-ACN]; gradient: 36%-56% B over 10 min) and the eluent was concentrated to remove ACN and lyophilized to give I-816 (15.04 mg, 20.23 μmol, 14.58% yield, 98.317% purity) as a white solid. LCMS (Method D): Retention time: 0.417 min, [M+H]=731.3. ¹H NMR (400 MHz, METHANOL-d4) δ=8.76-8.68 (m, 1H), 8.39-8.32 (m, 1H), 8.19-8.15 (m, 1H), 7.96-7.90 (m, 1H), 7.89-7.78 (m, 2H), 7.51-7.40 (m, 2H), 7.36 (d, J=6.0 Hz, 1H), 7.17-7.13 (m, 1H), 4.37 (s, 2H), 3.73 (s, 2H), 3.68-3.46 (m, 12H), 3.35 (d, J=4.0 Hz, 1H), 3.29 (s, 1H), 2.80 (d, J=4.8 Hz, 2H), 1.51 (s, 9H). ¹⁹F NMR (377 MHz, METHANOL-d4) δ=−120.674.

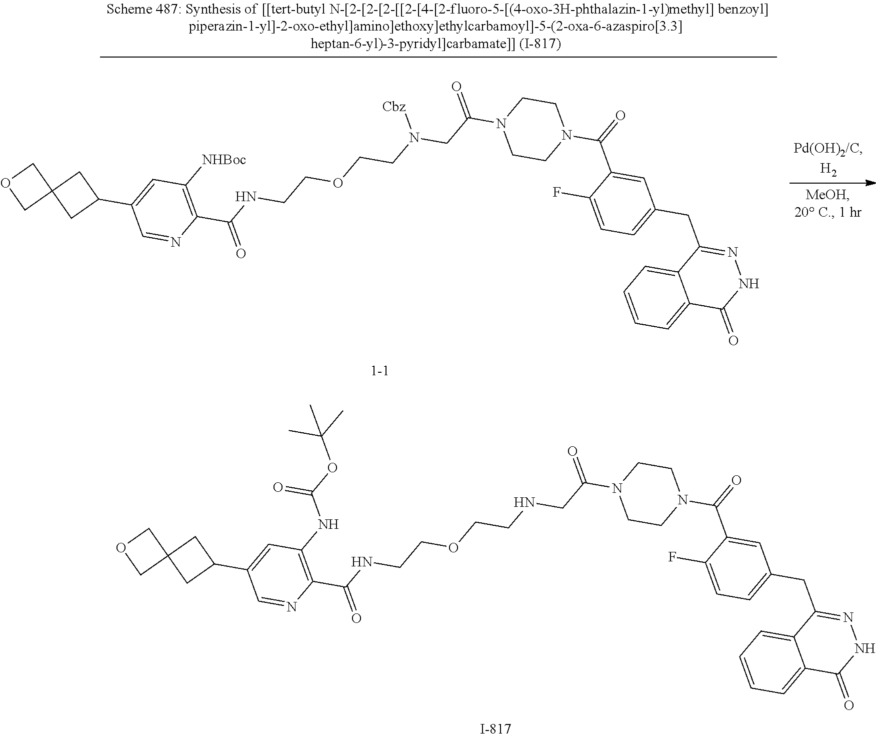 [SMILES: CC(C)(C)OC(=O)Nc1cc(C2CC3(COC3)C2)cnc1C(=O)NCCOCCN(CC(=O)N1CCN(C(=O)c2cc(Cc3n[nH]c(=O)c4ccccc34)ccc2F)CC1)C(=O)OCc1ccccc1 ; CC(C)(C)OC(=O)Nc1cc(C2CC3(COC3)C2)cnc1C(=O)NCCOCCNCC(=O)N1CCN(C(=O)c2cc(Cc3n[nH]c(=O)c4ccccc34)ccc2F)CC1 ; "I-817"]

To a mixture of Intermediate 1-1 (150 mg, 155.92 μmol, 1 eq) in MeOH (3 mL) was added Pd (OH)₂/C (54.74 mg, 77.96 μmol, 20% purity, 0.5 eq). The suspension was degassed and purged with N₂ 3 times, and then the mixture was degassed and purged with H₂ 3 times. Then the mixture was stirred at 20° C. for 1 hour under H₂ atmosphere (15 psi). The reaction mixture was filtered and concentrated under reduced pressure to give a residue. The residue was purified by reversed phase chromatography (0.100 FA condition). The eluent was concentrated under reduced pressure to remove MeCN and then lyophilized to give I-817 (12.69 mg, 15.09 μmol, 24.99% yield, 98.449% purity) as a white solid. LCMS (Method D): Retention time: 0.376 min [M+H]⁺=828.3. ¹H NMR (400 MHz, METHANOL-d4) δ=8.36 (d, J=7.6 Hz, 1H), 7.97-7.90 (m, 1H), 7.89-7.78 (m, 2H), 7.69 (s, 1H), 7.52-7.41 (m, 2H), 7.35 (d, J=1.6 Hz, 1H), 7.20-7.11 (m, 1H), 4.83 (s, 2H), 4.38 (d, J=3.2 Hz, 2H), 4.17 (s, 4H), 3.72 (s, 2H), 3.62 (s, 5H), 3.55-3.45 (m, 6H), 3.36 (d, J=4.4 Hz, 1H), 3.30 (s, 4H), 2.85-2.76 (m, 2H), 1.51 (s, 9H). ¹⁹F NMR (376 MHz, METHANOL-d4) δ=−120.66.

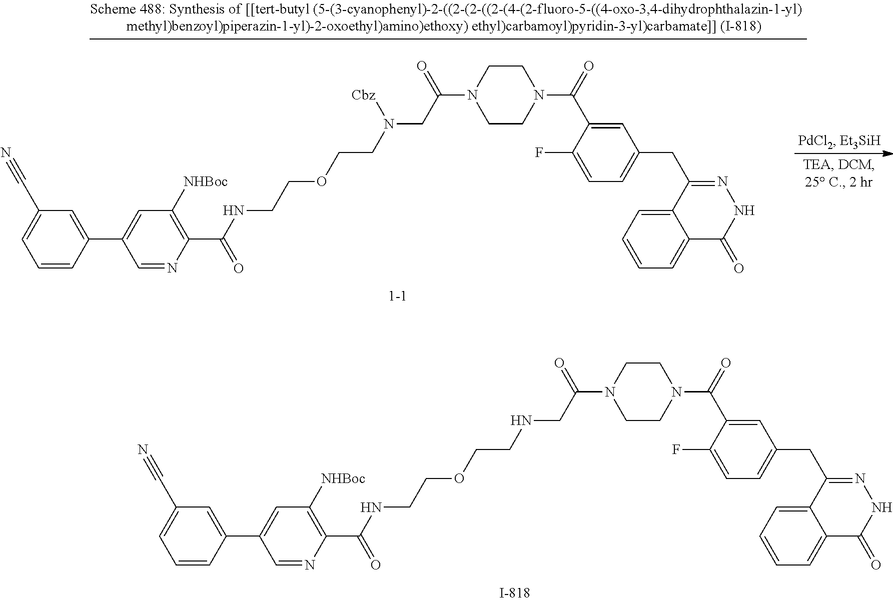 [SMILES: CC(C)(C)OC(=O)Nc1cc(-c2cccc(C#N)c2)cnc1C(=O)NCCOCCN(CC(=O)N1CCN(C(=O)c2cc(Cc3n[nH]c(=O)c4ccccc34)ccc2F)CC1)C(=O)OCc1ccccc1 ; CC(C)(C)OC(=O)Nc1cc(-c2cccc(C#N)c2)cnc1C(=O)NCCOCCNCC(=O)N1CCN(C(=O)c2cc(Cc3n[nH]c(=O)c4ccccc34)ccc2F)CC1]

To a solution of Intermediate 1-1 (30 mg, 31.06 μmol, 1 eq) in DCM (0.3 mL) was added PdCl₂ (2.75 mg, 15.53 μmol, 0.5 eq), TEA (9.43 mg, 93.17 μmol, 12.97 μL, 3 eq) and Et₃SiH (43.33 mg, 372.66 μmol, 59.52 μL, 12 eq). The mixture was stirred at 25° C. for 1 hr. The reaction mixture was concentrated under reduced pressure to give a residue. The crude product was purified by reversed phase chromatography (0.1% TFA condition), and the eluent was concentrated to remove ACN and lyophilized to give product. I-818 (19.01 mg, 22.70 μmol, 73.08% yield, 99.319% purity) was obtained as a white solid. LCMS (Method D): Retention time: 0.394 min, [M+H]⁺=832.2. ¹H NMR (400 MHz, METHANOL-d4) δ=9.05 (s, 1H), 8.56-8.50 (m, 1H), 8.36 (d, J=7.2 Hz, 1H), 8.09 (s, 1H), 8.01 (d, J=7.6 Hz, 1H), 7.96-7.91 (m, 1H), 7.90-7.77 (m, 3H), 7.76-7.65 (m, 1H), 7.48 (s, 1H), 7.34 (s, 1H), 7.14 (d, J=4.4 Hz, 1H), 4.37 (s, 2H), 3.77-3.65 (m, 8H), 3.62 (s, 3H), 3.53-3.47 (m, 2H), 3.36 (d, J=8.0 Hz, 1H), 3.28-3.23 (m, 1H), 3.03-2.85 (m, 2H), 1.54 (s, 9H). ¹⁹F NMR (377 MHz, METHANOL-d4) δ=−120.667.

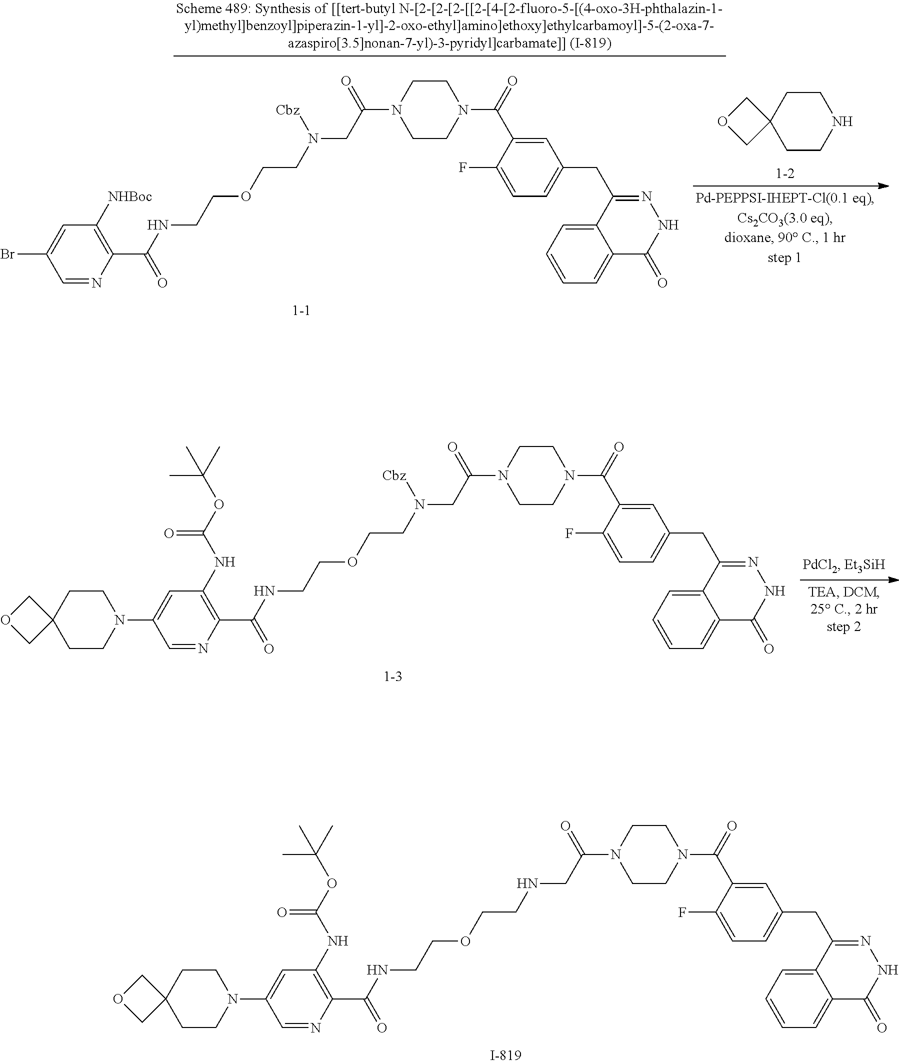 [SMILES: CC(C)(C)OC(=O)Nc1cc(Br)cnc1C(=O)NCCOCCN(CC(=O)N1CCN(C(=O)c2cc(Cc3n[nH]c(=O)c4ccccc34)ccc2F)CC1)C(=O)OCc1ccccc1 ; C1CC2(CCN1)COC2 ; CC(C)(C)OC(=O)Nc1cc(N2CCC3(CC2)COC3)cnc1C(=O)NCCOCCN(CC(=O)N1CCN(C(=O)c2cc(Cc3n[nH]c(=O)c4ccccc34)ccc2F)CC1)C(=O)OCc1ccccc1 ; CC(C)(C)OC(=O)Nc1cc(N2CCC3(CC2)COC3)cnc1C(=O)NCCOCCNCC(=O)N1CCN(C(=O)c2cc(Cc3n[nH]c(=O)c4ccccc34)ccc2F)CC1]

### Step 1: Synthesis of Intermediate 1-3

To a solution of intermediate 1-1 (500 mg, 529.77 μmol, 1 eq) and intermediate 1-2 (202.13 mg, 1.59 mmol, 3 eq) in dioxane (6 mL) was added Cs₂CO₃ (517.82 mg, 1.59 mmol, 3 eq) and Pd-PEPPSI-IHEPT-Cl (51.53 mg, 52.98 μmol, 0.1 eq), then the mixture was stirred at 90° C. for 1 hour under N₂ atmosphere. The reaction mixture was diluted with water (40 mL) and extracted with EA (30 mL*2). The combined organic layers were concentrated under reduced pressure to give a residue. The crude product was purified by reversed phase chromatography (0.1% FA condition). The eluent was concentrated under reduced pressure to remove ACN and then lyophilized to give the product. Intermediate 1-3 (410 mg, 386.26 μmol, 72.91% yield, 93.275% purity) was obtained as a yellow solid. LCMS (Method D): Rt=0.543 min, [M+H]⁺=990.4.

### Step 2: Synthesis of I-819

To a solution of intermediate 1-3 (20 mg, 20.20 μmol, 1 eq) in DCM (1 mL) was added PdCl₂ (716.41 g, 4.04 μmol, 0.2 eq) and TEA (4.09 mg, 40.40 μmol, 5.62 μL, 2 eq). The reaction mixture was degassed with N₂. Then a mixture of Et₃SiH (9.40 mg, 80.80 μmol, 12.91 μL, 4 eq) in DCM (0.5 mL) was added dropwise at 10° C. The mixture was stirred at 25° C. for 2 hours. The reaction mixture was concentrated under reduced pressure to give a residue. The crude product was purified by reversed phase chromatography (0.1% FA condition). The eluent was concentrated under reduced pressure to remove ACN and then lyophilized to give the product. I-819 (13.69 mg, 14.97 μmol, 74.09% yield, 98.608% purity, FA salt) was obtained as a white solid. LCMS (Method D): Rt=0.399 min, [M+H]⁺=856.3. ¹H NMR (400 MHz, METHANOL-d4) δ=8.51 (s, 1H), 8.37 (d, J=7.6 Hz, 1H), 8.21 (s, 1H), 7.94 (d, J=8.4 Hz, 2H), 7.89-7.80 (m, 2H), 7.50 (s, 1H), 7.35 (d, J=5.2 Hz, 1H), 7.20-7.12 (m, 1H), 4.49 (d, J=7.6 Hz, 4H), 4.39 (s, 2H), 3.88 (s, 1H), 3.80 (s, 1H), 3.77-3.62 (m, 8H), 3.59-3.42 (m, 5H), 3.34 (d, J=5.2 Hz, 5H), 3.08 (d, J=4.0 Hz, 2H), 1.97 (d, J=4.0 Hz, 4H), 1.51 (s, 9H). ¹⁹F NMR (377 MHz, METHANOL-d4) δ=−120.605.

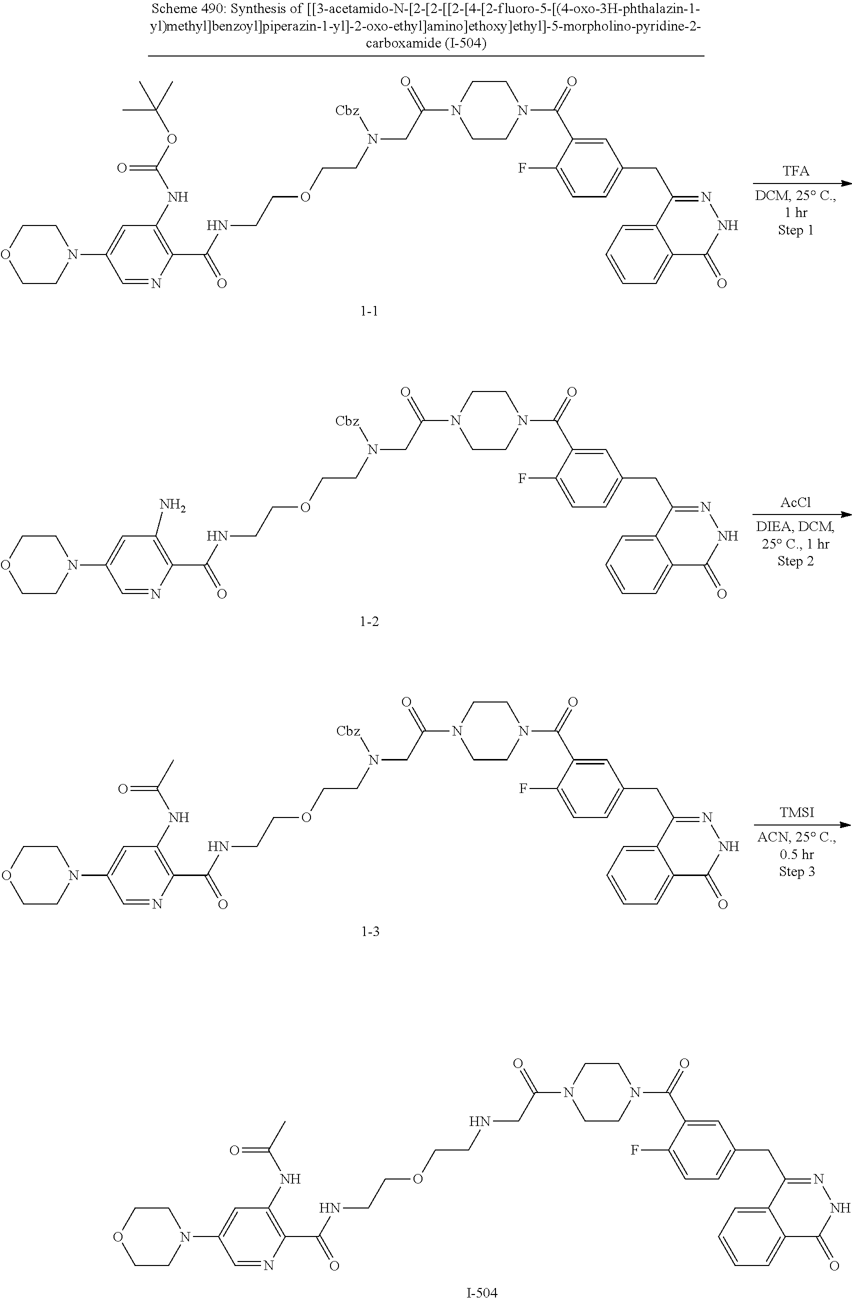

### Step 1: Synthesis of Intermediate 1-2

To a mixture of Intermediate 1-1 (100 mg, 105.26 μmol, 1 eq) in DCM (0.5 mL) was added TFA (767.50 mg, 6.73 mmol, 0.5 mL, 63.95 eq). Then the mixture was stirred at 25° C. for 1 hr. The mixture was concentrated under reduced pressure to give a residue. The crude product was used in the next step without further purification. Intermediate 1-2 (100 mg, crude, TFA salt) was obtained as a yellow solid. LCMS (Method D): Retention time=0.420 min, [M+H]⁺=850.3.

### Step 2: Synthesis of Intermediate 1-3

To a mixture of Intermediate 1-2 (100 mg, 103.74 μmol, 1 eq, TFA salt) and acetyl chloride (8.14 mg, 103.74 μmol, 7.38 μL, 1 eq) in DCM (1 mL) was added DIEA (26.82 mg, 207.48 μmol, 36.14 μL, 2 eq) at 25° C. Then the mixture was stirred at 25° C. for 1 hr. The reaction mixture was diluted with water (1 mL) and extracted with DCM (1 mL*3), the combined organic phase was dried by Na₂SO₄, and concentrated under reduced pressure to give a residue. The residue was purified by flash silica gel chromatography (ISCO; 12 g SepaFlash Silica Flash Column, Eluent of 0˜50% Methanol/Dichloromethane @35 mL/min) and the eluent was concentrated to give product. Intermediate 1-3 (96 mg, 91.26 mol, 87.97% yield, 84.788% purity) was obtained as a white solid. LCMS (Method D): Retention time=0.431 min, [M+H]⁺=892.4.

### Step 3: Synthesis of I-504

To a solution of Intermediate 1-3 (68 mg, 76.24 μmol, 1 eq) in ACN (0.7 mL) was added TMSI (45.76 mg, 228.71 μmol, 31.13 μL, 3 eq), and the reaction mixture was stirred at 25° C. for 0.5 hr. The reaction mixture was diluted with water (1 mL) and extracted with DCM (1 mL*3), the combined organic phase was dried by Na₂SO₄, and concentrated under reduced pressure to give a residue. The crude was purified by prep-HPLC (column: Welch Xtimate C18 150*30 mm*5 um; mobile phase: [water (FA)-ACN]; gradient: 99%-39% B over 10 min) and the eluent was concentrated to remove ACN and lyophilized to give products. I-504 (19 mg, 22.02 μmol, 28.88% yield, 93.155% purity, FA salt) was obtained as a white solid. LCMS (Method D): Retention time=0.318 min, [M+H]⁺=758.3. ¹H NMR (400 MHz, METHANOL-d4) δ=8.58 (s, 1H), 8.55 (s, 1H), 8.44 (d, J=7.6 Hz, 1H), 8.07 (d, J=8.8 Hz, 1H), 8.05-7.99 (m, 1H), 7.98-7.87 (m, 2H), 7.62-7.55 (m, 1H), 7.45 (d, J=5.6 Hz, 1H), 7.26 (t, J=8.8 Hz, 1H), 4.46 (s, 2H), 4.18-4.03 (m, 2H), 3.90 (s, 6H), 3.85-3.77 (m, 4H), 3.75 (s, 1H), 3.70 (s, 2H), 3.62-3.51 (m, 2H), 3.39 (d, J=4.0 Hz, 9H), 2.28 (d, J=2.0 Hz, 3H). ¹⁹F NMR (377 MHz, METHANOL-d₄), δ=−120.560.

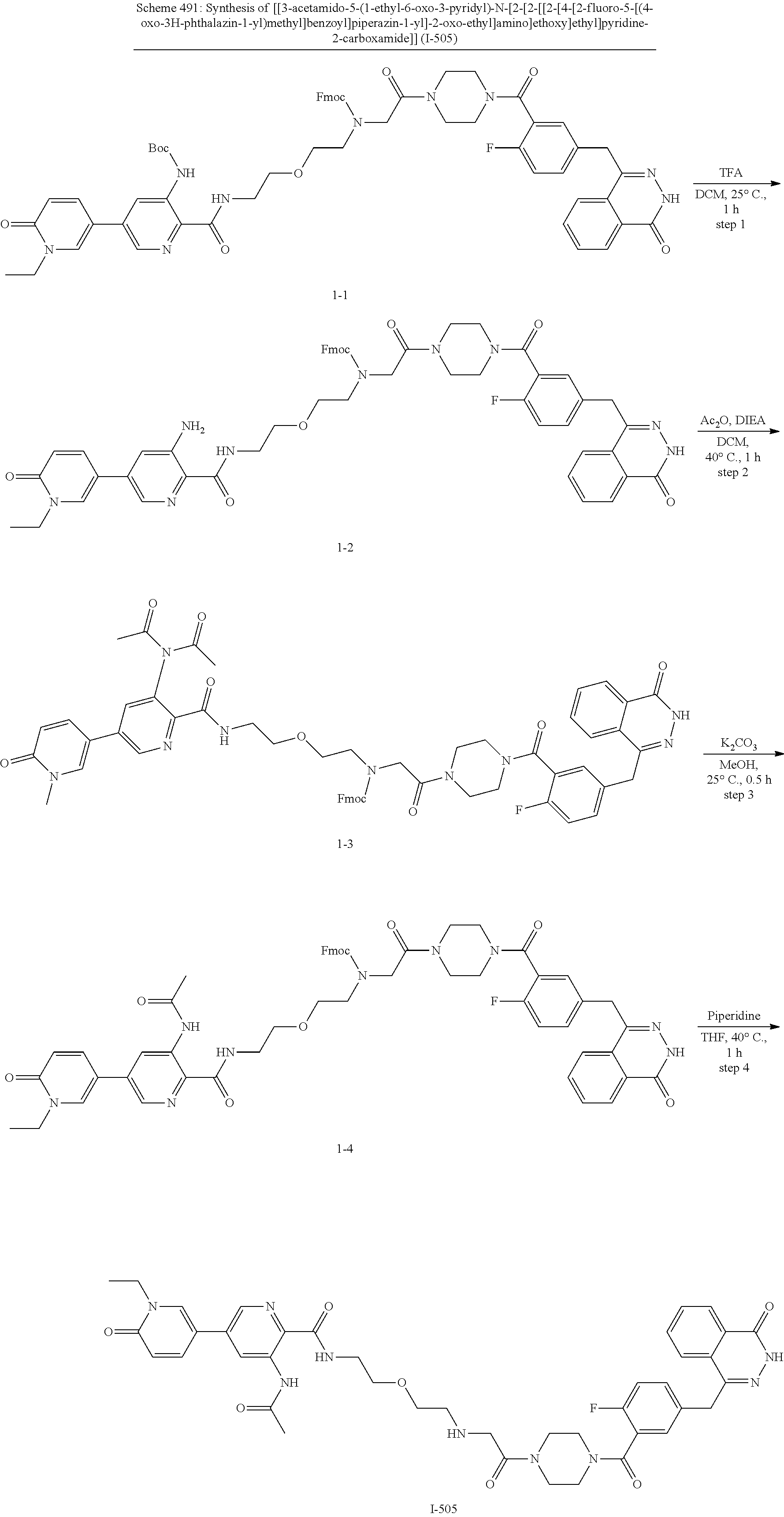

### Step 1: Synthesis of Intermediate 1-2

To a solution of Intermediate 1-1 (400 mg, 372.38 μmol, 1 eq) in DCM (2 mL) was added TFA (3.07 g, 26.92 mmol, 2 mL, 72.30 eq). The mixture was stirred at 25° C. for 1 hr. The reaction mixture was concentrated under reduce pressure to give a residue. The residue was purified by reversed phase chromatography (0.1% FA condition). The eluent was lyophilized to give product. Intermediate 1-2 (320 mg, 294.10 μmol, 78.98% yield, TFA salt) was obtained as a yellow solid. LCMS (Method D): Rt=0.431 min, [M+H]⁺=974.3.

### Step 2: Synthesis of Intermediate 1-3

To a solution of Intermediate 1-2 (180 mg, 165.43 μmol, 1 eq, TFA salt) and acetyl acetate (25.33 mg, 248.15 μmol, 23.31 μL, 1.5 eq) in DCM (2 mL) was added DIEA (64.14 mg, 496.29 μmol, 86.44 μL, 3 eq). The mixture was stirred at 25° C. for 12 hr. The reaction mixture was concentrated under reduce pressure to give a residue. The residue was used for the next step without further purification. Intermediate 1-3 (150 mg, 141.76 μmol, 85.69% yield) was obtained as a yellow oil. LCMS (Method): Rt=2.332 min, [M+H]⁺=1058.7.

### Step 3: Synthesis of Intermediate 1-4

To a solution of Intermediate 1-3 (150 mg, 141.76 μmol, 1 eq) in MeOH (2 mL) was added K₂CO₃ (39.19 mg, 283.52 μmol, 2 eq). The mixture was stirred at 25° C. for 0.5 hr. The reaction mixture was concentrated under reduce pressure to give a residue. The residue was used for the next step without further purification. Intermediate 1-4 (140 mg, 137.78 μmol, 97.19% yield) was obtained as a yellow oil. LCMS (Method): Rt=2.228 min.

### Step 4: Synthesis of I-505

To a solution of Intermediate 1-4 (140 mg, 137.78 μmol, 1 eq) in THF (1.6 mL) was added piperidine (172.44 mg, 2.03 mmol, 0.2 mL, 14.70 eq). The mixture was stirred at 40° C. for 0.5 hr. The reaction mixture was concentrated under reduced pressure to give a residue. The residue was purified by reversed phase chromatography (0.1% NH₃·H₂O). Then the eluent was lyophilized to give product. I-505 (16.31 mg, 20.07 μmol, 53.12% yield, 97.707% purity) was obtained as a yellow solid. LCMS (Method D): Rt=0.327 min, [M+H]⁺=794.4. ¹H NMR (400 MHz, METHANOL-d4) δ=9.11 (s, 1H), 8.49 (s, 1H), 8.35 (d, J=7.6 Hz, 1H), 8.15 (s, 1H), 7.96-7.90 (m, 1H), 7.90-7.78 (m, 3H), 7.47 (s, 1H), 7.34 (s, 1H), 7.14 (t, J=8.8 Hz, 1H), 6.66 (t, J=8.8 Hz, 1H), 4.36 (s, 2H), 4.11 (s, 2H), 3.74-3.59 (m, 9H), 3.57-3.41 (m, 5H), 3.36 (s, 1H), 3.25 (s, 1H), 2.81 (s, 2H), 2.23 (s, 3H), 1.37 (d, J=5.2 Hz, 3H). ¹⁹F NMR (377 MHz, METHANOL-d₄), δ=120.68.

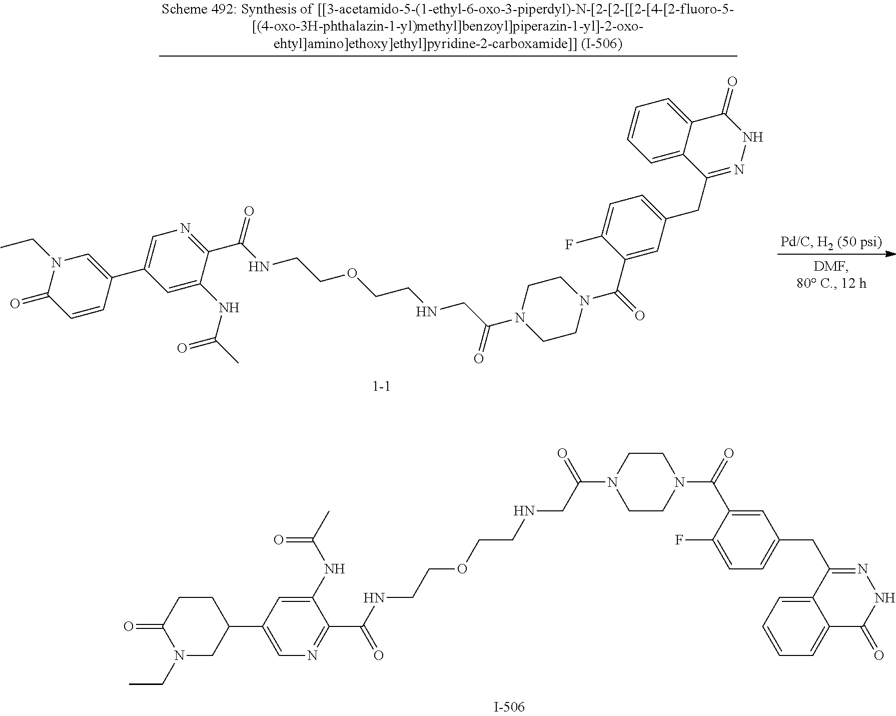 [SMILES: CCn1cc(-c2cnc(C(=O)NCCOCCNCC(=O)N3CCN(C(=O)c4cc(Cc5n[nH]c(=O)c6ccccc56)ccc4F)CC3)c(NC(C)=O)c2)ccc1=O ; "Pd/C, H2 (50 psi)" ; CCN1CC(c2cnc(C(=O)NCCOCCNCC(=O)N3CCN(C(=O)c4cc(Cc5n[nH]c(=O)c6ccccc56)ccc4F)CC3)c(NC(C)=O)c2)CCC1=O]

A mixture of Intermediate 1-1 (20 mg, 25.19 μmol, 1 eq) and Pd/C (10 mg, 10% purity) in DMF (1 mL) was degassed and purged with H₂ 3 times, and then the mixture was stirred at 80° C. for 9 hr under H₂ atmosphere (50 Psi). The reaction mixture was filtered through a pad of celite and washed with MeOH (3 mL*3), filtered and concentrated under reduced pressure to give a residue. The residue was purified by prep-HPLC (column: Welch Xtimate C18 150*25 mm*5 um; mobile phase: [water(FA)-ACN]; gradient: 10%-30% B over 15 min). The eluent was lyophilized to give product. I-506 (4 mg, 4.68 μmol, 18.56% yield, 98.654% purity, FA salt) was obtained as a white solid. LCMS (Method D): Rt=0.333 min, [M+H]⁺=798.3. ¹H NMR (400 MHz, METHANOL-d4) δ=8.98 (s, 1H), 8.46 (s, 1H), 8.37 (d, J=7.2 Hz, 1H), 8.30 (s, 1H), 7.98-7.92 (m, 1H), 7.91-7.80 (m, 2H), 7.51 (s, 1H), 7.36 (s, 1H), 7.22-7.13 (m, 1H), 4.39 (s, 2H), 4.04-3.90 (m, 2H), 3.86-3.58 (m, 10H), 3.57-3.37 (m, 7H), 3.18 (s, 2H), 2.49 (s, 2H), 2.21 (s, 3H), 2.09 (s, 2H), 1.41-1.23 (m, 2H), 1.14 (d, J=6.4 Hz, 3H). ¹⁹F NMR (377 MHz, METHANOL-d4), δ=120.63. SFC: Retention time: 4.092 min, 4.704 min.

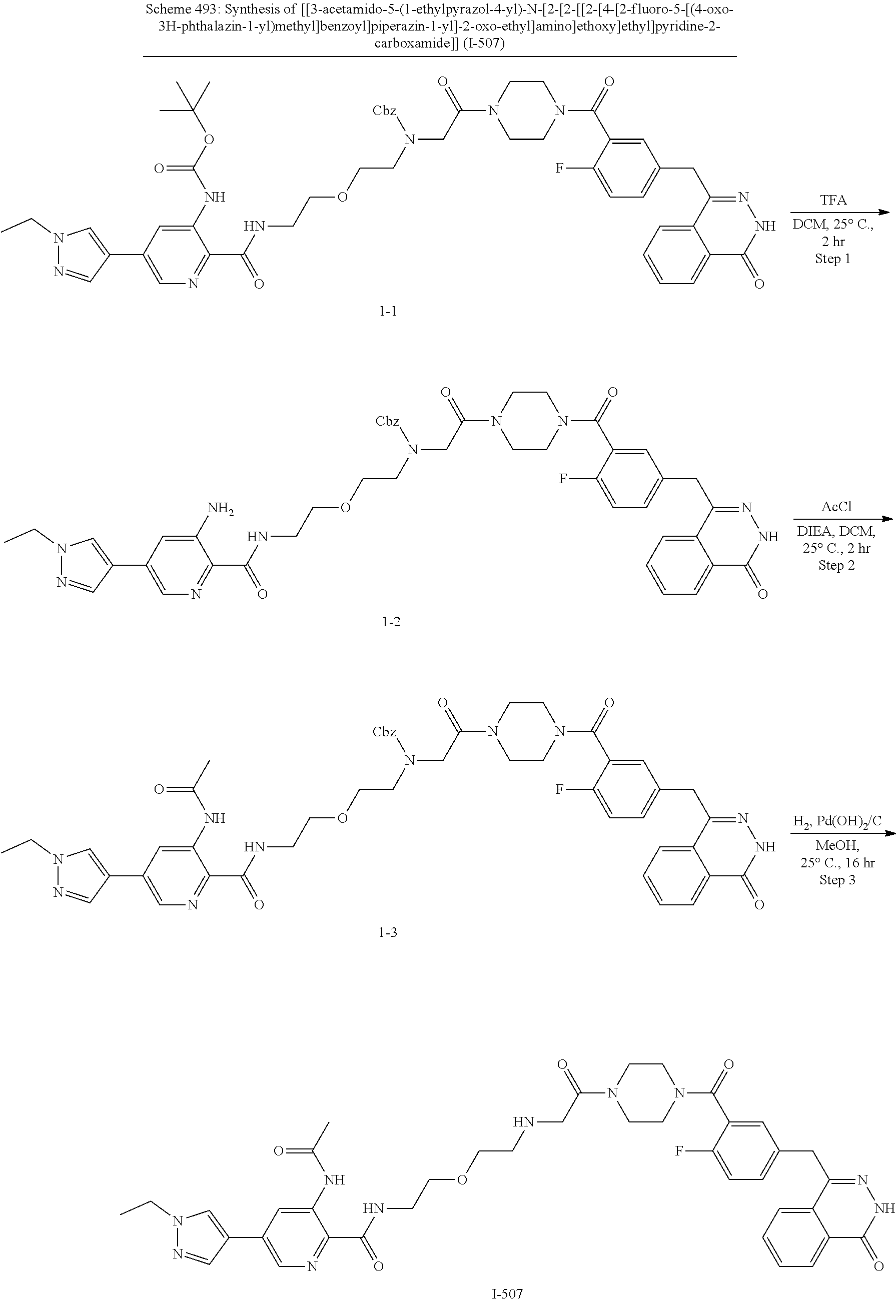

### Step 1: Synthesis of Intermediate 1-2

To a solution of intermediate 1-1 (120 mg, 125.13 μmol, 1 eq) in DCM (1.2 mL) was added TFA (460.50 mg, 4.04 mmol, 0.3 mL, 32.28 eq). The mixture was stirred at 25° C. for 1 hr. The reaction mixture was filtered and concentrated under reduced pressure to give a residue. The crude product was used in the next step without further purification. Intermediate 1-2 (230 mg, crude) was obtained as a white solid. LCMS (Method D): Rt=0.404 min, [M+H]⁺=859.2.

### Step 2: Synthesis of Intermediate 1-3

To a solution of intermediate 1-2 (210 mg, 244.49 μmol, 1 eq) and acetyl chloride (19.19 mg, 244.49 μmol, 17.38 μL, 1 eq) in DCM (2.1 mL) was added DIEA (94.80 mg, 733.48 μmol, 127.76 μL, 3 eq). Then the mixture was stirred at 25° C. for 1 hr. The reaction mixture was filtered and concentrated under reduced pressure to give a residue. The residue was purified by flash silica gel chromatography (ISCO; 20 g SepaFlash Silica Flash Column, Eluent of 0˜68% Ethylacetate/Methanol @50 mL/min), the eluent was concentrated under reduced pressure to give the product. Intermediate 1-3 (150 mg, 166.49 μmol, 68.10% yield) was obtained as a yellow oil. LCMS (Method D): Rt=0.413 min, [M+H]⁺=901.3.

### Step 3: Synthesis of I-507

To a solution of intermediate 1-3 (50 mg, 55.50 μmol, 1 eq) in MeOH (0.5 mL) was added Pd(OH)₂/C (25 mg, 20% purity) under N₂ atmosphere. The suspension was degassed and purged with H₂ 3 times and stirred under H₂ (15 psi) at 25° C. for 16 hr. The reaction mixture was filtered and concentrated under reduced pressure to give a residue. The crude product was purified by reversed phase chromatography (0.1% TFA condition) followed by lyophilization to give product. I-507 (10.95 mg, 12.36 μmol, 22.28% yield, 99.446% purity, TFA salt) was obtained as a white solid. LCMS (Method D): Rt=0.360 min, [M+H]⁺=767.3. ¹H NMR (400 MHz, METHANOL-d4) δ=9.12 (d, J=3.6 Hz, 1H), 8.52 (d, J=10.0 Hz, 1H), 8.36 (d, J=7.6 Hz, 1H), 8.19 (s, 1H), 7.98-7.91 (m, 2H), 7.90-7.78 (m, 2H), 7.56-7.45 (m, 1H), 7.38-7.28 (m, 1H), 7.21-7.09 (m, 1H), 4.38 (d, J=4.4 Hz, 2H), 4.28-4.19 (m, 2H), 4.14 (s, 1H), 4.07 (s, 1H), 3.92-3.80 (m, 2H), 3.77-3.69 (m, 4H), 3.66 (s, 3H), 3.46 (d, J=20.0 Hz, 3H), 3.29 (s, 3H), 3.16-2.86 (m, 1H), 2.27-2.19 (m, 3H), 1.51-1.45 (m, 3H). ¹⁹F NMR (400 MHz, METHANOL-d₄), δ=−77.17, −120.68.

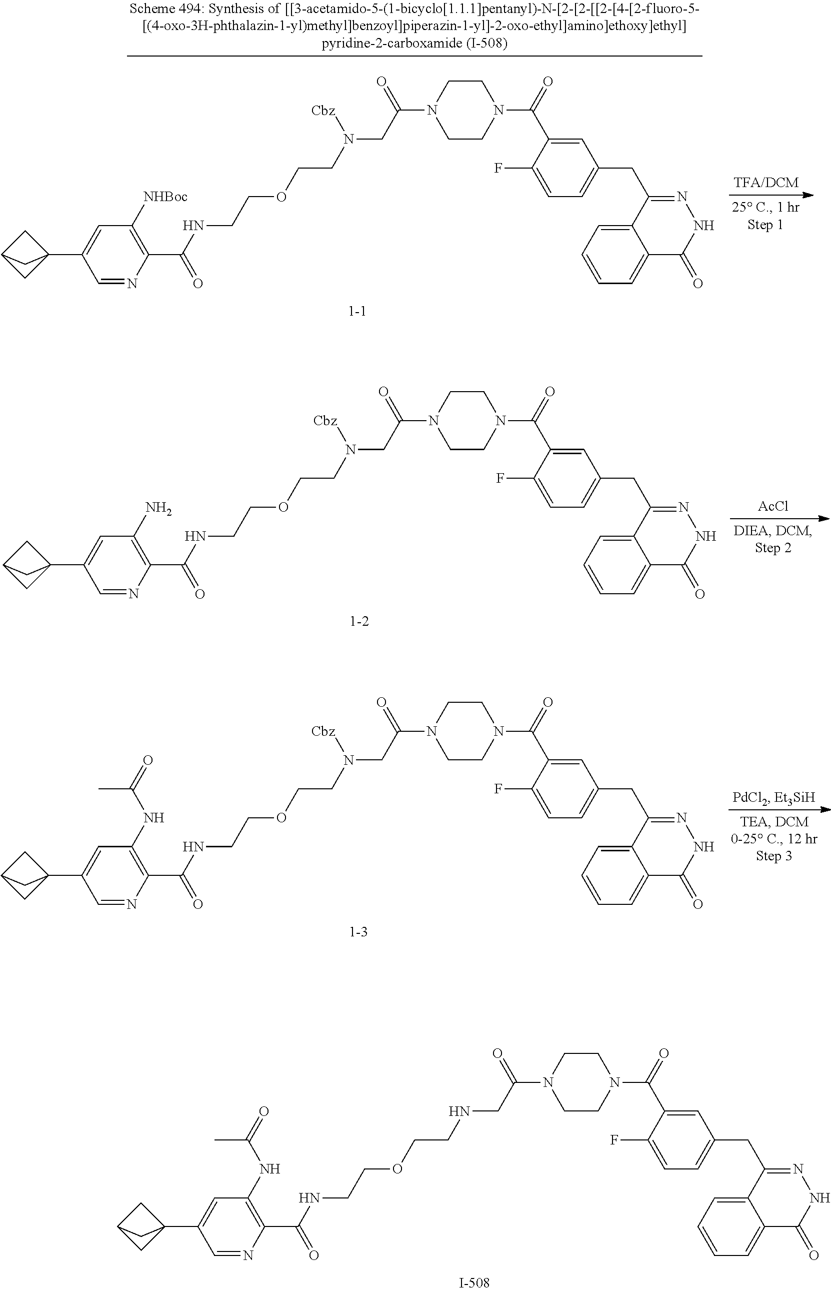

### Step 1: Synthesis of Intermediate 1-2

To a solution of Intermediate 1-1 (110 mg, 118.15 μmol, 1 eq) in DCM (1 mL) was added TFA (767.50 mg, 6.73 mmol, 0.5 mL, 56.97 eq). The mixture was stirred at 25° C. for 1 hr. The mixture was concentrated to give Intermediate 1-2 (110 mg, crude, TFA salt) as a brown oil. LCMS (Method D): Rt=0.470 min, [M+H]⁺=831.2.

### Step 2: Synthesis of Intermediate 1-3

To a solution of Intermediate 1-2 (100 mg, 105.83 μmol, 1 eq, TFA salt) in DCM (1 mL) was added acetyl chloride (9.97 mg, 126.99 μmol, 9.03 μL, 1.2 eq) and DIEA (41.03 mg, 317.49 μmol, 55.30 L, 3 eq). The mixture was stirred at 25° C. for 1 hr. The reaction mixture was diluted with water (30 mL) and extracted with ethyl acetate (10 mL×3). The combined organic layers were washed with brine (10 mL×3) and dried over sodium sulfate, filtered and concentrated under reduced pressure to give Intermediate 1-3 (50 mg, crude) as a brown solid. LCMS (Method D): Rt=0.481 min, [M+H]⁺=873.3.

### Step 3: Synthesis of I-508

To a solution of Intermediate 1-3 (60 mg, 68.73 μmol, 1 eq) in DCM (1 mL) was added PdCl₂ (3.66 mg, 20.62 μmol, 0.3 eq). The suspension was degassed and purged with N₂ 3 times. Then TEA (20.87 mg, 206.20 μmol, 28.70 μL, 3 eq) and Et₃SiH (39.96 mg, 343.67 μmol, 54.89 μL, 5 eq) was added into the mixture and stirred at 25° C. for 1 hr. The reaction mixture was diluted with water (10 mL) and extracted with DCM (10 mL×3). The combined organic layers were washed with brine (10 mL×3) and dried over sodium sulfate, filtered and concentrated under reduced pressure to give a residue. The crude product was purified by reversed phase chromatography (0.1% FA condition). The eluent was concentrated under reduced pressure to remove ACN and then lyophilized to give I-508 (13.08 mg, 15.71 μmol, 22.85% yield, 94.234% purity, FA salt) as a brown solid. LCMS (Method D): Rt=0.366 min, [M+H]⁺=739.2. ¹H NMR (400 MHz, METHANOL-d4) δ=8.80 (s, 1H), 8.48 (s, 1H), 8.36 (d, J=7.2 Hz, 1H), 8.14 (s, 1H), 7.97-7.91 (m, 1H), 7.91-7.77 (m, 2H), 7.50 (s, 1H), 7.36 (s, 1H), 7.17-7.16 (m, 1H), 4.38 (s, 2H), 3.99 (s, 1H), 3.94-3.87 (m, 1H), 3.81-3.68 (m, 6H), 3.63 (s, 3H), 3.48 (s, 2H), 3.31-3.23 (m, 3H), 3.17 (s, 2H), 2.58 (d, J=2.4 Hz, 1H), 2.25-2.12 (m, 9H). ¹⁹F NMR (400 MHz, METHANOL-d4) δ=−120.637.

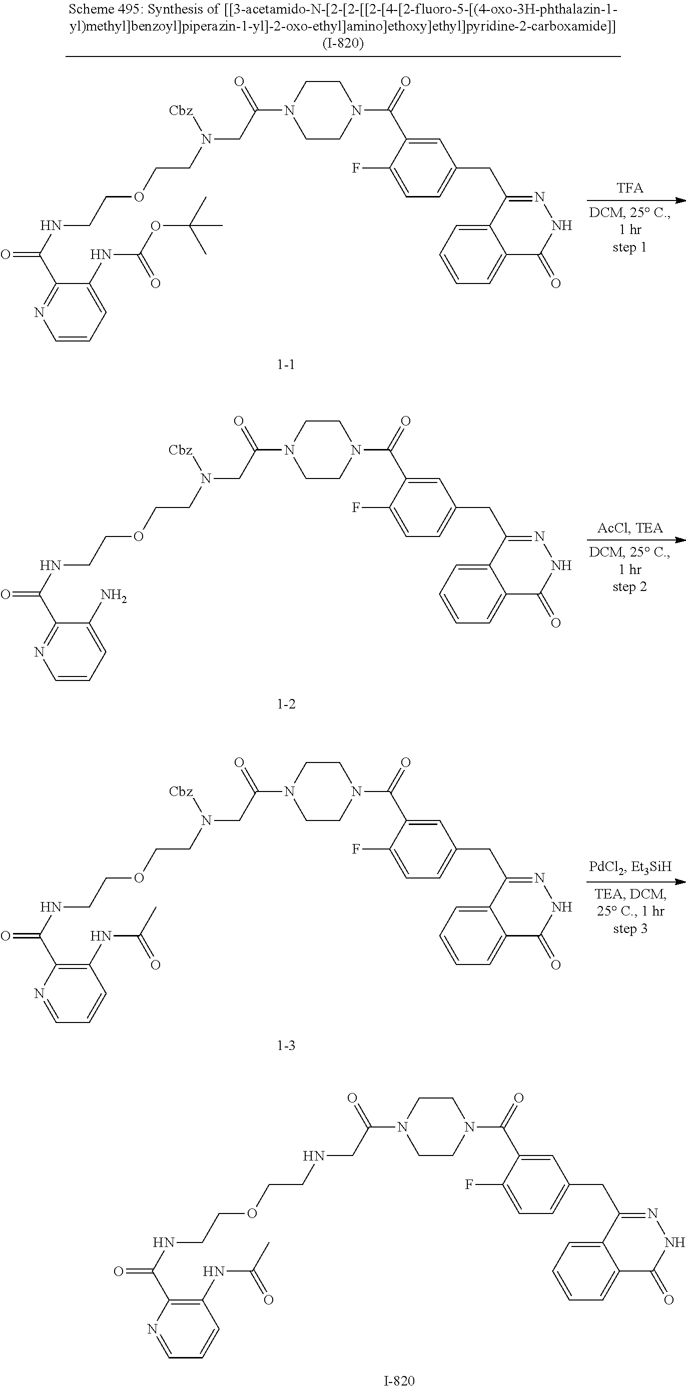 [SMILES: CC(C)(C)OC(=O)Nc1cccnc1C(=O)NCCOCCN(CC(=O)N1CCN(C(=O)c2cc(Cc3n[nH]c(=O)c4ccccc34)ccc2F)CC1)C(=O)OCc1ccccc1 ; Nc1cccnc1C(=O)NCCOCCN(CC(=O)N1CCN(C(=O)c2cc(Cc3n[nH]c(=O)c4ccccc34)ccc2F)CC1)C(=O)OCc1ccccc1 ; CC(=O)Nc1cccnc1C(=O)NCCOCCN(CC(=O)N1CCN(C(=O)c2cc(Cc3n[nH]c(=O)c4ccccc34)ccc2F)CC1)C(=O)OCc1ccccc1 ; CC(=O)Nc1cccnc1C(=O)NCCOCCNCC(=O)N1CCN(C(=O)c2cc(Cc3n[nH]c(=O)c4ccccc34)ccc2F)CC1]

### Step 1: Synthesis of Intermediate 1-2

To a solution of Intermediate 1-1 (230 mg, 265.92 μmol, 1 eq) in DCM (2 mL) was added TFA (614.00 mg, 5.38 mmol, 0.4 mL). The mixture was stirred at 25° C. for 0.5 hr. The mixture was dried with N₂ to give a crude product. The crude product was used in the next step. Intermediate 1-2 (200 mg, 157.66 μmol, 59.29% yield, 69.276% purity, TFA salt) was obtained as a yellow solid. LCMS (Method D): Rt=0.421 min, [M+H]⁺=765.2.

### Step 2: Synthesis of Intermediate 1-3

To a solution of Intermediate 1-2 (200 mg, 227.58 μmol, 1 eq, TFA salt) in DCM (2 mL) was added TEA (46.06 mg, 455.15 μmol, 63.35 μL, 2 eq) and acetyl chloride (17.86 mg, 227.58 μmol, 16.18 L, 1 eq). The mixture was stirred at 25° C. for 0.5 hr. The reaction mixture was quenched by addition of H₂O (2 mL) at 25° C. and extracted with DCM (1 mL*3), dried over Na₂SO₄, filtered and concentrated under reduced pressure to give a residue. The crude product was purified by reversed phase chromatography (0.1% FA condition) and concentrated under reduced pressure to give the product. Intermediate 1-3 (140 mg, 125.28 μmol, 55.05% yield, 72.201% purity) was obtained as a yellow solid. LCMS (Method D): Rt: 0.457 min, [M+H]⁺=807.4.

### Step 3: Synthesis of I-830

To a solution of Intermediate 1-3 (140 mg, 173.52 μmol, 1 eq) in DCM (1.5 mL) was added PdCl₂ (15.38 mg, 86.76 μmol, 0.5 eq) and TEA (52.67 mg, 520.55 μmol, 72.45 μL, 3 eq). The reaction mixture was purged with N₂. Then Et₃SiH (242.11 mg, 2.08 mmol, 332.57 μL, 12 eq) was added in. The mixture was stirred at 25° C. for 1 hr. The reaction mixture was quenched by addition of H₂O (1.5 mL) at 25° C., and extracted with DCM (0.5 mL*3), dried over Na₂SO₄, filtered and concentrated under reduced pressure to give a crude product. The crude product was purified by reversed phase chromatography (0.1% FA condition) and lyophilized to give the product. I-820 (20.63 mg, 26.80 μmol, 15.45% yield, 93.384% purity, FA salt) was obtained as a yellow solid. LCMS (Method D): Rt: 0.311 min, [M+H]⁺=673.1. ¹H NMR (400 MHz, METHANOL-d₄) δ=8.99 (d, J=5.2 Hz, 1H), 8.47 (s, 1H), 8.39 (d, J=8.0 Hz, 1H), 8.30 (d, J=9.2 Hz, 1H), 8.01-7.93 (m, 1H), 7.93-7.81 (m, 2H), 7.52 (s, 2H), 7.38 (s, 1H), 7.19 (t, J=8.8 Hz, 1H), 4.41 (s, 2H), 4.02 (s, 1H), 3.94 (s, 1H), 3.83-3.61 (m, 10H), 3.51 (d, J=6.4 Hz, 4H), 3.19 (s, 2H), 2.23 (s, 3H). ¹⁹F NMR (376 MHz, METHANOL-d₄) δ=−120.642.

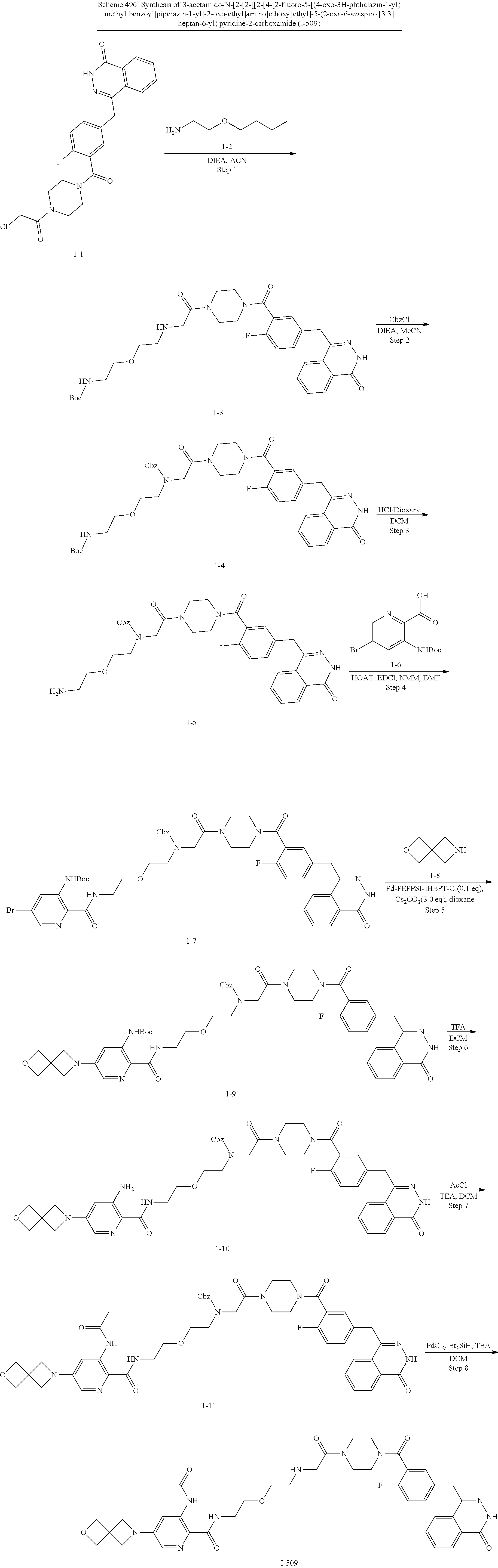

### Step 1: Synthesis of Intermediate 1-3.

To a solution of Intermediate 1-1 (6 g, 13.55 mmol, 1 eq) and Intermediate 1-2 (2.77 g, 13.55 mmol, 1 eq) in ACN (60 mL) was added DIEA (5.25 g, 40.64 mmol, 7.08 mL, 3 eq). Then the mixture was stirred at 40° C. for 2 hours. The reaction mixture was concentrated under reduced pressure to give Intermediate 1-3 (7 g, crude) as a yellow solid. LCMS (Method D): Retention time: 0.315 min, [M+H]⁺=611.4.

### Step 2: Synthesis of Intermediate 1-4.

To a mixture of Intermediate 1-3 (6 g, 9.83 mmol, 1 eq) in ACN (60 mL) was added DIEA (2.54 g, 19.65 mmol, 3.42 mL, 2 eq) and CbzCl (2.51 g, 14.74 mmol, 2.10 mL, 1.5 eq). Then the mixture was stirred at 20° C. for 1 hour. The reaction mixture was concentrated under reduced pressure to give a residue. The crude product was purified by reversed phase chromatography (0.1% FA condition). The eluent was concentrated under reduced pressure to remove MeCN and lyophilized to give Intermediate 1-4 (7.3 g, 9.02 mmol, 91.78% yield, 92% purity) as a yellow oil. LCMS (Method D): Retention time: 0.448 min, [M+H]⁺=745.5. ¹H NMR (400 MHz, CHLOROFORM-d) δ=10.48-10.28 (m, 1H), 8.51-8.43 (m, 1H), 7.83-7.67 (m, 3H), 7.37 (s, 2H), 7.36-7.34 (m, 5H), 7.05 (t, J=8.8 Hz, 1H), 5.16 (s, 2H), 5.11 (s, 3H), 4.88 (d, J=1.6 Hz, 2H), 4.29 (s, 2H), 4.25-4.09 (m, 2H), 3.59-3.48 (m, 7H), 3.40 (d, J=5.2 Hz, 2H), 3.30 (d, J=4.4 Hz, 2H), 1.44 (s, 9H).

### Step 3: Synthesis of Intermediate 1-5.

To a mixture of Intermediate 1-4 (6 g, 8.06 mmol, 1 eq) in DCM (30 mL) was added HCl/dioxane (2 M, 4.03 mL, 1 eq). Then the mixture was stirred at 20° C. for 0.5 hour. The reaction mixture was concentrated under reduced pressure to give Intermediate 1-5 (5.6 g, crude, HCl salt) as a white solid. LCMS (Method D): Retention time: 0.321 min, [M+H]⁺=645.4.

### Step 4: Synthesis of Intermediate 1-7.

To a mixture of Intermediate 1-5 (3.5 g, 5.14 mmol, 1.1 eq, HCl salt) and Intermediate 1-6 (1.48 g, 4.67 mmol, 1 eq) in DMF (40 mL) was added HOAt (635.81 mg, 4.67 mmol, 653.45 μL, 1 eq), EDCI (2.69 g, 14.01 mmol, 3 eq), and NMM (2.36 g, 23.36 mmol, 2.57 mL, 5 eq). Then the mixture was stirred at 20° C. for 16 hours. The reaction mixture was diluted with water (50 mL) and extracted with EA (10 mL*3). The combined organic layers were concentrated under reduced pressure to give a residue. The residue was purified by flash silica gel chromatography (ISCO; 60 g SepaFlash Silica Flash Column, Eluent of 10% Ethyl acetate/MeOH @80 mL/min). The eluent was concentrated under reduced pressure to give a residue. Intermediate 1-7 (2.5 g, 2.65 mmol, 56.71% yield) as a white solid. LCMS (Method D): Retention time: 0.534 min, [M+H]⁺=943.3. ¹H NMR (400 MHz, CHLOROFORM-d) δ=11.17 (s, 1H), 10.22 (d, J=10.4 Hz, 1H), 9.00 (d, J=16.4 Hz, 1H), 8.41 (d, J=6.8 Hz, 1H), 8.30 (s, 1H), 8.07 (d, J=18.4 Hz, 1H), 7.78-7.62 (m, 3H), 7.29-7.28 (m, 1H), 7.26-7.18 (m, 5H), 6.99 (t, J=8.4 Hz, 1H), 5.13-4.99 (m, 2H), 4.30-4.00 (m, 4H), 3.76-3.44 (m, 13H), 3.33-3.19 (m, 2H), 3.16-2.97 (m, 1H), 1.46 (s, 9H). ¹⁹F NMR (376 MHz, METHANOL-d4) δ=−117.52.

### Step 5: Synthesis of Intermediate 1-9.

To a mixture of Intermediate 1-7 (150 mg, 158.93 μmol, 1 eq) and Intermediate 1-8 (47.26 mg, 476.79 μmol, 3 eq) in dioxane (2 mL) was added Cs₂CO₃ (155.35 mg, 476.79 μmol, 3 eq) and Pd-PEPPSI-IHEPT-Cl (15.46 mg, 15.89 μmol, 0.1 eq). Then the mixture was stirred at 90° C. for 1 hour under N₂. The reaction mixture was diluted with water (30 mL) and extracted with EA (5 mL*3). The combined organic layers were concentrated under reduced pressure to give a residue. The residue was purified by flash silica gel chromatography (ISCO; 20 g SepaFlash Silica Flash Column, Eluent of 0˜10% Ethyl acetate/MeOH ether gradient @60 mL/min). The eluent was concentrated under reduced pressure to give Intermediate 1-9 (120 mg, 124.74 μmol, 78.48% yield) as a white solid. LCMS (Method D): Retention time: 0.451, [M+H]⁺=962.0.

### Step 6: Synthesis of Intermediate 1-10.

A mixture of Intermediate 1-9 (80 mg, 83.16 μmol, 1 eq) in TFA (0.5 mL) and DCM (0.5 mL) was stirred at 20° C. for 1 hour. The reaction mixture was concentrated under reduced pressure to give Intermediate 1-10 (80 mg, 81.97 μmol, 98.57% yield, TFA salt) as a white solid. LCMS (Method D): Retention time: 0.423 min, [M+H]⁺=862.5.

### Step 7: Synthesis of Intermediate 1-11.

To a mixture of Intermediate 1-10 (80 mg, 81.97 μmol, 1 eq, TFA aslt) in DCM (1 mL) was added TEA (24.88 mg, 245.92 μmol, 34.23 μL, 3 eq) and acetyl chloride (6.43 mg, 81.97 μmol, 5.83 μL, 1 eq), then the mixture was stirred at 0° C. for 5 min. The reaction mixture was quenched with water (15 mL) and extracted with DCM (10 mL*2). The combined organic layers were concentrated under reduced pressure to give Intermediate 1-11 (60 mg, 66.38 μmol, 80.97% yield) as a white solid. LCMS (Method D): Retention time: 0.446 min, [M+H]⁺=904.3.

### Step 8: Synthesis of I-509

To a mixture of Intermediate 1-11 (50 mg, 55.31 μmol, 1 eq) in DCM (2 mL) was added PdCl₂ (2.94 mg, 16.59 μmol, 0.3 eq) and TEA (11.19 mg, 110.63 μmol, 15.40 μL, 2 eq). The suspension was degassed and purged with N₂. Then a solution of Et₃SiH (25.73 mg, 221.25 μmol, 35.34 μL, 4 eq) in DCM (1 mL) was added to the mixture dropwise. Then the mixture was stirred at 20° C. for 1 hour under N₂. The reaction mixture was filtered and concentrated under reduced pressure to give a residue. The crude product was purified by reversed phase chromatography (0.1% FA condition). The eluent was concentrated under reduced pressure to remove MeCN and then lyophilized to give a residue. I-509 (10.38 mg, 12.51 μmol, 22.61% yield, 98.3% purity, FA salt) was obtained as a yellow solid. LCMS (Method D): Retention time: 0.324 min, [M+H]⁺=770.3. ¹H NMR (400 MHz, METHANOL-d4) δ=8.47 (s, 1H), 8.37 (d, J=7.6 Hz, 1H), 7.95 (d, J=10.8 Hz, 2H), 7.90-7.81 (m, 2H), 7.55-7.47 (m, 2H), 7.35 (d, J=5.6 Hz, 1H), 7.19-7.13 (m, 1H), 4.83-4.80 (m, 4H), 4.39 (s, 2H), 4.17 (s, 4H), 3.98 (s, 1H), 3.90 (s, 1H), 3.81-3.61 (m, 8H), 3.59 (s, 2H), 3.50 (s, 1H), 3.41 (s, 1H), 3.28 (s, 2H), 3.19-3.15 (m, 2H), 2.20-2.18 (m, 3H). ¹⁹F NMR (377 MHz, METHANOL-d4) δ=−120.66.

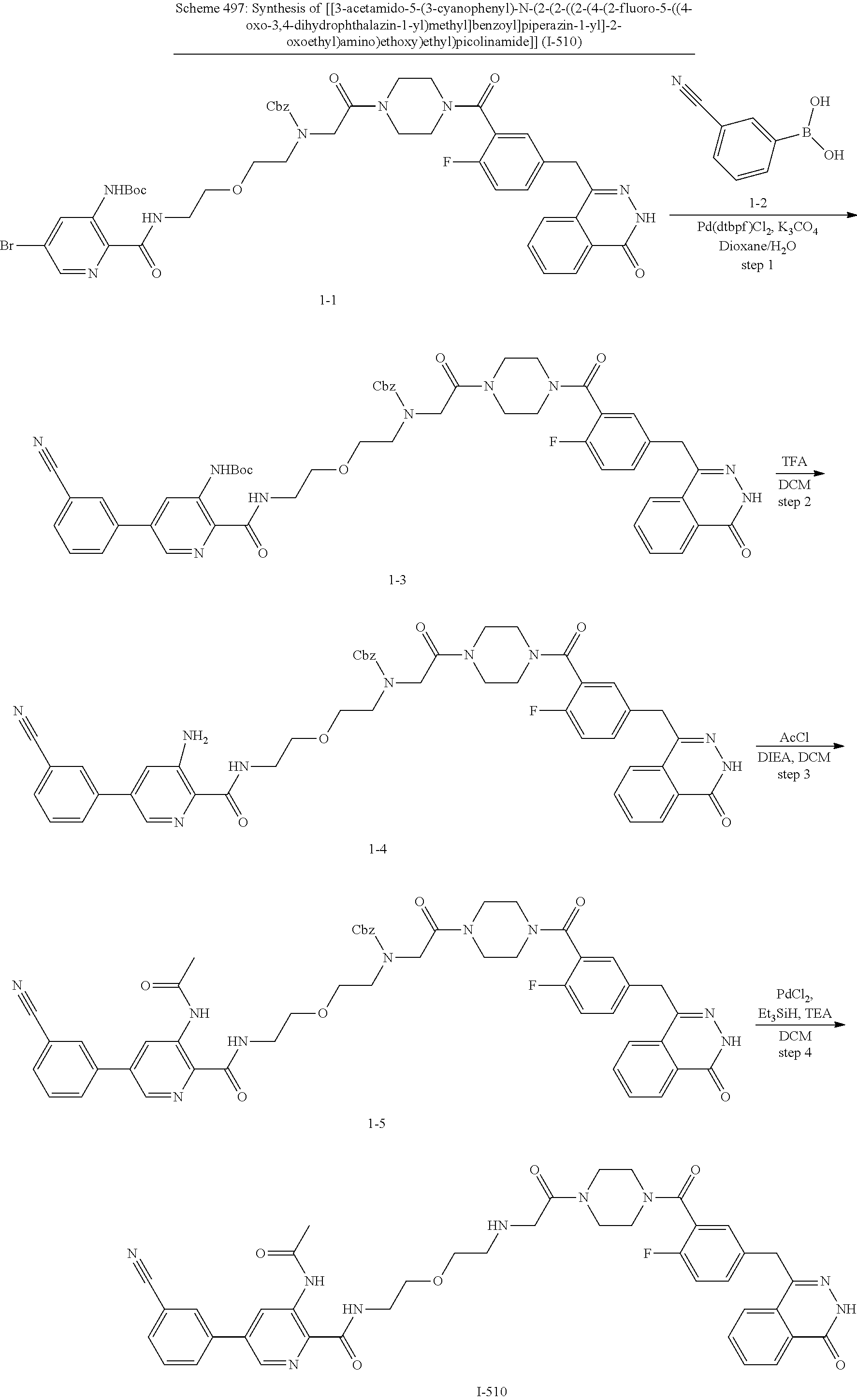

### Step 1: Synthesis of Intermediate 1-3

To a solution of Intermediate 1-1 (270 mg, 286.07 μmol, 1 eq) and Intermediate 1-2 (50.44 mg, 343.29 μmol, 1.2 eq) in dioxane (3 mL) and H₂O (0.6 mL) was added Pd(dtbpf)Cl₂ (18.64 mg, 28.61 μmol, 0.1 eq) and K₃PO₄ (182.17 mg, 858.22 μmol, 3 eq). The mixture was stirred at 80° C. for 1 hr. The reaction mixture was filtered and the filtrate was purified by flash silica gel chromatography. The residue was purified by flash silica gel chromatography (ISCO; 40 g SepaFlash Silica Flash Column, Eluent of 0˜100% Ethyl acetate/Petroleum ethergradient @50 mL/min). Then the eluent was concentrated to give product. Intermediate 1-3 (260 mg, 269.15 μmol, 94.08% yield) was obtained as a white solid. LCMS (Method D): Retention time: 0.487 min, [M+H]⁺=966.3.

### Step 2: Synthesis of Intermediate 1-4

To a solution of Intermediate 1-3 (100 mg, 103.52 μmol, 1 eq) in DCM (0.9 mL) was added TFA (460.50 mg, 4.04 mmol, 0.3 mL, 39.02 eq). The mixture was stirred at 25° C. for 1 hr. The reaction mixture was concentrated under reduced pressure to give a residue. The crude product was used in the next step. Intermediate 1-4 (100 mg, 102.05 μmol, 98.58% yield, TFA salt) was obtained as a white solid. LCMS (Method D): Retention time: 0.424 min, [M+H]⁺=866.3.

### Step 3: Synthesis of Intermediate 1-5

To a solution of Intermediate 1-4 (100 mg, 102.05 μmol, 1 eq, TFA salt) in DCM (1 mL) was added DIEA (39.57 mg, 306.14 μmol, 53.32 μL, 3 eq) and acetyl chloride (9.61 mg, 122.46 μmol, 8.71 μL, 1.2 eq). The mixture was stirred at 0° C. for 1 hr. The reaction mixture was concentrated under reduced pressure to give a residue. The crude product was purified by reversed phase chromatography (0.1% TFA condition), then the eluent was concentrated to remove ACN and lyophilized to give the product. Intermediate 1-5 (35 mg, 38.55 μmol, 37.77% yield) was obtained as a white solid. LCMS (Method D): Retention time: 0.432 min, [M+H]⁺=908.3.

### Step 4: Synthesis of I-510

To a solution of Intermediate 1-5 (35 mg, 38.55 μmol, 1 eq) in DCM (0.4 mL) was added PdCl₂ (3.42 mg, 19.27 μmol, 0.5 eq), TEA (11.70 mg, 115.65 μmol, 16.10 μL, 3 eq) and Et₃SiH (53.79 mg, 462.58 μmol, 73.88 μL, 12 eq). The mixture was stirred at 25° C. for 5 hr. The reaction mixture was concentrated under reduced pressure to give a residue. The crude product was purified by reversed phase chromatography (0.10% TFA condition), then the eluent was concentrated to remove ACN and lyophilized to give the product. I-510 (6.03 mg, 6.23 μmol, 16.17% yield, 91.754% purity, TFA salt) was obtained as a white solid. LCMS (Method D): Retention time: 0.333 min, [M+H]⁺=774.3. ¹H NMR (400 MHz, METHANOL-d4) δ=9.29 (s, 1H), 8.62-8.60 (m, 1H), 8.36-8.34 (m, 1H), 8.08 (s, 1H), 8.00 (d, J=8.0 Hz, 1H), 7.96-7.90 (m, 1H), 7.90-7.76 (m, 3H), 7.73-7.67 (m, 1H), 7.51-7.48 (m, 1H), 7.39-7.27 (m, 1H), 7.15 (t, J=9.2 Hz, 1H), 4.37 (s, 2H), 4.19-4.05 (m, 2H), 3.83 (d, J=4.4 Hz, 2H), 3.74 (s, 4H), 3.68 (s, 3H), 3.56-3.39 (m, 3H), 3.33-3.31 (m, 2H), 3.28 (s, 2H), 2.25 (d, J=3.2 Hz, 3H). ¹⁹F NMR (400 MHz, METHANOL-d₄), δ=−120.67, −77.06.

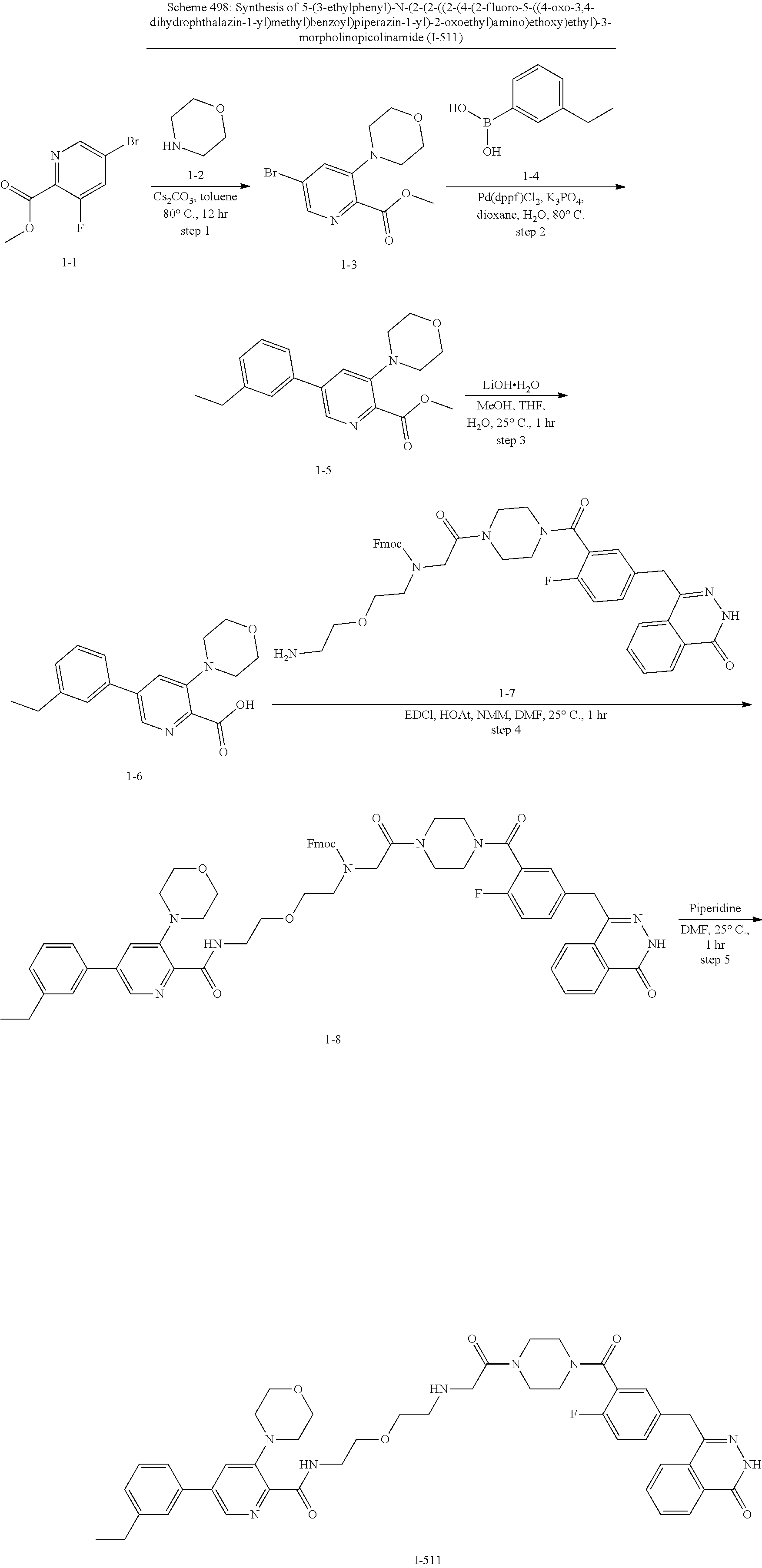

### Step 1: Synthesis of Intermediate 1-3

To a solution of Intermediate 1-1 (200 mg, 854.62 μmol, 1 eq) and Intermediate 1-2 (74.45 mg, 854.62 μmol, 75.21 μL, 1 eq) in toluene (2 mL) was added Cs₂CO₃ (835.35 mg, 2.56 mmol, 3 eq). The mixture was stirred at 60° C. for 1 hr. The crude product with filtered to remove Cs₂CO₃. The filtrate was concentrated. Intermediate 1-3 (200 mg, 664.15 μmol, 77.71% yield) was obtained as a white solid. LCMS (Method D): Retention time: 0.276 min, [M+H]⁺=301.0.

### Step 2: Synthesis of Intermediate 1-5

To a solution of Intermediate 1-3 (180 mg, 597.74 μmol, 1 eq) and Intermediate 1-4 (134.47 mg, 896.60 μmol, 1.5 eq) in dioxane (2 mL) and H₂O (0.4 mL) was added Pd(dtbpf)Cl₂ (38.96 mg, 59.77 μmol, 0.1 eq) and K₃PO₄ (380.64 mg, 1.79 mmol, 3 eq). Then the reaction mixture was degassed and purged with N₂ 3 times and stirred at 80° C. for 1 hr under N₂ atmosphere. The reaction mixture was filtered and the filtrate was concentrated to afford a residue. The residue was purified by flash silica gel chromatography (ISCO; 20 g SepaFlash Silica Flash Column, Eluent of 0˜100% Ethyl acetate/Petroleum ether gradient @30 mL/min) and the eluent was concentrated to give product. Intermediate 1-5 (300 mg, 61% purity) was obtained as a white solid. LCMS (Method D): Retention time: 0.557 min, [M+H]⁺=327.1.

### Step 3: Synthesis of Intermediate 1-6

To a solution of Intermediate 1-5 (270 mg, 827.23 μmol, 1 eq) in THF (1 mL), MeOH (1 mL) and H₂O (1 mL) was added LiOH·H₂O (69.43 mg, 1.65 mmol, 2 eq). The mixture was stirred at 25° C. for 1 hr. The aqueous phase was adjusted to pH=7 with HCl, and then extracted with DCM (5 mL*3). The combined organic phase was dried by Na₂SO₄, filtered and concentrated. Intermediate 1-6 (200 mg, 640.28 μmol, 77.40% yield) was obtained as a white solid and used in the next step without further purification. LCMS (Method D): Retention time: 0.384 min, [M+H]⁺=313.4.

### Step 4: Synthesis of Intermediate 1-8

To a solution of Intermediate 1-6 (32.48 mg, 104.00 μmol, 1 eq) and Intermediate 1-7 (80 mg, 104.00 μmol, 1 eq, HCl salt) in DMF (0.8 mL) was added EDCI (39.87 mg, 207.99 μmol, 2 eq), HOAt (14.16 mg, 104.00 μmol, 14.55 μL, 1 eq) and NMM (52.59 mg, 519.98 μmol, 57.17 μL, 5 eq). The mixture was stirred at 25° C. for 1 hr. The reaction mixture was diluted with H₂O (2 mL) and then extracted with DCM (2 mL*3). The combined organic phase was dried by Na₂SO₄, filtered and concentrated. The crude product was purified by reversed-phase chromatography (0.1% FA condition), and the eluent was concentrated to remove ACN and lyophilized to give product. Intermediate 1-8 (100 mg, 97.36 μmol, 93.62% yield) was obtained as a white solid. LCMS (Method D): Retention time: 0.455 min, [M+H]⁺=1003.2.

### Step 5: Synthesis of I-511

To a solution of Intermediate 1-8 (80 mg, 77.89 μmol, 1 eq) in DMF (0.8 mL) was added piperidine (13.26 mg, 155.77 μmol, 15.38 μL, 2 eq). The mixture was stirred at 25° C. for 1 hr. The reaction mixture was diluted H₂O (2 mL) and then extracted with DCM (2 mL*3). The combined organic phase was washed, dried by Na₂SO₄, filtered and concentrated. The crude product was purified by reversed-phase chromatography (0.1% FA condition), and the eluent was concentrated to remove ACN and lyophilized to give product. I-511 (18.39 mg, 21.61 μmol, 27.75% yield, 100.000% purity, FA salt) was obtained as a white solid. LCMS (Method D): Retention time: 0.340 min, [M+H]⁺=805.4. ¹H NMR (400 MHz, METHANOL-d4) δ=8.50 (s, 1H), 8.44 (d, J=5.2 Hz, 1H), 8.37-8.35 (m, 1H), 7.97-7.90 (m, 1H), 7.89-7.79 (m, 2H), 7.75 (s, 1H), 7.53-7.45 (m, 3H), 7.44-7.25 (m, 3H), 7.16-7.11 (m, 1H), 4.37 (s, 2H), 3.92-3.80 (m, 6H), 3.74 (d, J=4.4 Hz, 5H), 3.67 (d, J=4.4 Hz, 4H), 3.46 (s, 2H), 3.34 (s, 1H), 3.23 (s, 1H), 3.15 (d, J=2.8 Hz, 4H), 3.08 (d, J=4.4 Hz, 2H), 2.74-2.69 (m, 2H), 1.30-1.23 (m, 3H). ¹⁹F NMR (377 MHz, METHANOL-d4) δ=−120.674.

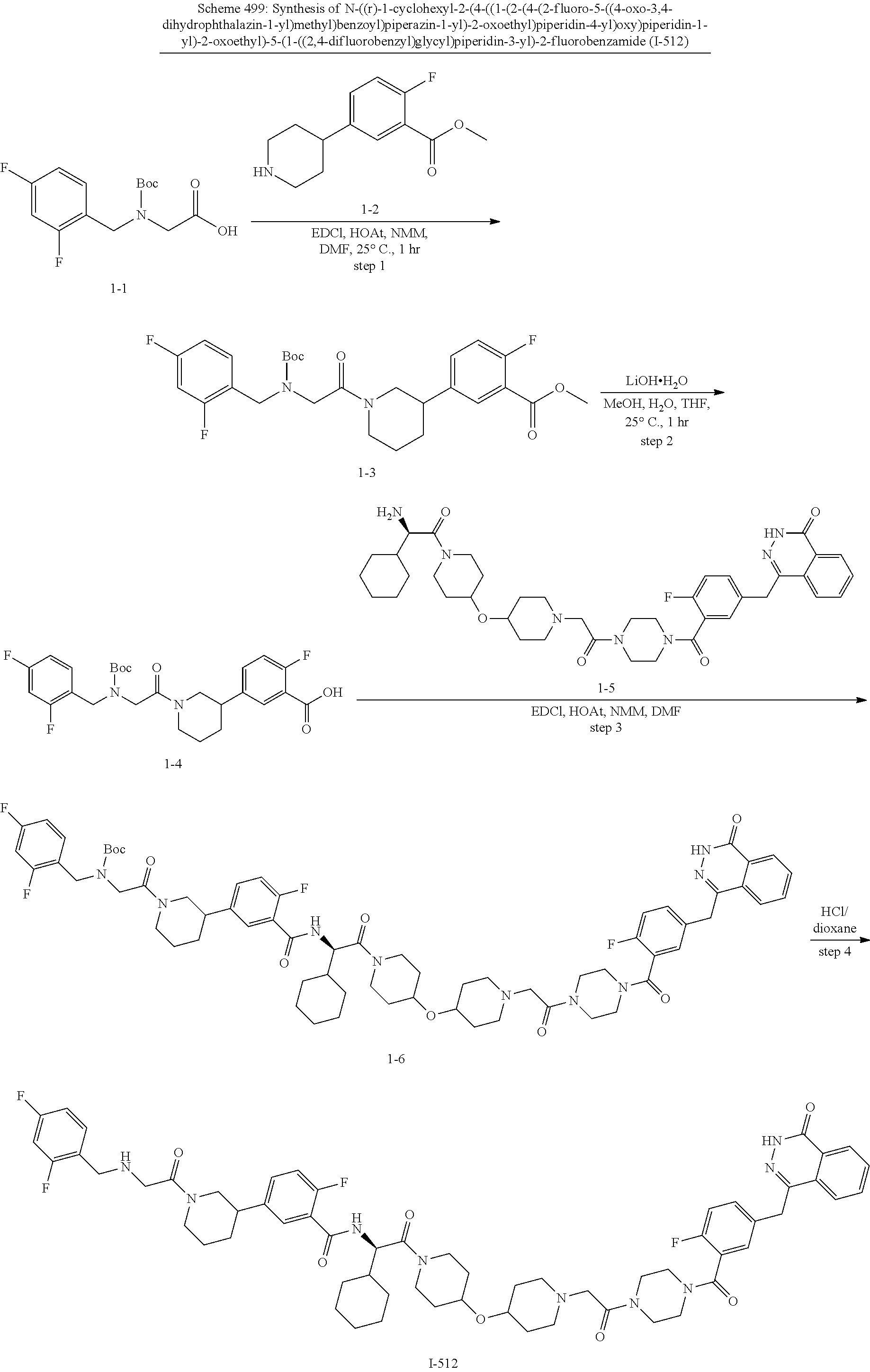

### Step 1: Synthesis of Intermediate 1-3

To a solution of Intermediate 1-1 (200 mg, 663.82 μmol, 1 eq) in DMF (4 mL) was added HOAt (90.35 mg, 663.82 μmol, 92.86 μL, 1 eq), EDCI (254.51 mg, 1.33 mmol, 2 eq), NMM (335.72 mg, 3.32 mmol, 364.91 μL, 5 eq) and Intermediate 1-2 (181.71 mg, 663.82 μmol, 1 eq, HCl salt). The mixture was stirred at 25° C. for 1 h. The reaction mixture was quenched by H₂O (10 mL) and extracted with EA (5 mL*3). The combined organic layers were washed with brine (5 mL*3), dried over anhydrous Na₂SO₄, filtered and concentrated under reduced pressure to give a crude product. The crude was used for the next step directly without purification. Intermediate 1-3 (400 mg, crude) was obtained as a yellow oil. LCMS (Method G): Rt=0.750 min, [M+Na]⁺=543.2.

### Step 2: Synthesis of Intermediate 1-4

To a solution of Intermediate 1-3 (400 mg, 768.43 μmol, 1 eq) in THF (3 mL), MeOH (3 mL) and H₂O (3 mL) was added LiOH·H₂O (64.49 mg, 1.54 mmol, 2 eq). The mixture was stirred at 25° C. for 1 h. The mixture was concentrated under vacuum to remove THF and MeOH. Then the aqueous layer was adjusted to pH=3 and extracted with EA (3 ml*3). The combined organic layer was washed with brine (3 ml*3), dried over anhydrous Na₂SO₄, filtered and concentrated under vacuum to a crude product. The crude was used for the next step directly without purification. Intermediate 1-4 (370 mg, crude) was obtained as a colorless oil. LCMS (Method G): Rt=0.412 min, [M+Na]⁺=529.3. ¹H NMR (400 MHz, DMSO-d₆) δ=13.65-12.00 (m, 1H), 8.00 (s, 1H), 7.79 (br s, 1H), 7.60 (br d, J=1.8 Hz, 1H), 7.44 (br d, J=7.3 Hz, 1H), 7.35-7.19 (m, 2H), 7.12 (br d, J=6.9 Hz, 1H), 4.58-4.32 (m, 3H), 4.22-4.12 (m, 1H), 4.06-3.95 (m, 1H), 3.86-3.76 (m, 1H), 3.18-3.05 (m, 1H), 2.73-2.65 (m, 1H), 1.77 (br s, 2H), 1.48-1.32 (m, 10H).

### Step 3: Synthesis of Intermediate 1-6

To a solution of Intermediate 1-4 (200 mg, 394.86 μmol, 1 eq) in DMF (4 mL) was added HOAt (53.74 mg, 394.86 μmol, 55.24 μL, 1 eq), EDCI (151.39 mg, 789.71 μmol, 2 eq), NMM (199.69 mg, 1.97 mmol, 217.06 μL, 5 eq) and Intermediate 1-5 (302.60 mg, 394.86 μmol, 1 eq, HCl salt). The mixture was stirred at 25° C. for 1 h. The mixture was poured into H₂O (10 mL), filtered and the filter cake was washed with H₂O (2 mL*3) and concentrated under vacuum to give a crude product. The crude was used for the next step without further purification. Intermediate 1-6 (450 mg, crude) was obtained as a yellow solid. LCMS (Method G): Rt=0.783 min, [M+H]⁺=1218.9.

### Step 4: Synthesis of I-512

To a solution of Intermediate 1-6 (450 mg, 369.34 μmol, 1 eq) in DCM (1.8 mL) was added HCl/dioxane (4 M, 1.80 mL, 19.49 eq). The mixture was stirred at 25° C. for 0.5 h. The reaction mixture was quenched by sat. NaHCO₃ (20 mL) and extracted with DCM (10 mL*3). The combined organic layers were washed with brine (10 mL*3) and dried over anhydrous Na₂SO₄, filtered and concentrated under reduced pressure to give a residue. The residue was purified by Prep-HPLC (column: Waters Xbridge 150*25 mm*5 um; mobile phase: [water (ammonia hydroxide v/v)-ACN]; gradient: 33%-63% B over 10 min). I-512 (134.35 mg, 118.66 μmol, 32.13% yield, 98.768% purity) was obtained as a white solid. LCMS (Method G): Rt=0.693 min, [M+H]⁺=1118.7. SFC: Rt=3.320 min, 3.677 min ¹H NMR (400 MHz, METHANOL-d₄) δ=8.37 (br d, J=7.4 Hz, 1H), 7.95 (br d, J=7.4 Hz, 1H), 7.91-7.80 (m, 2H), 7.65 (br d, J=5.6 Hz, 1H), 7.52-7.35 (m, 4H), 7.23-7.12 (m, 2H), 7.00-6.88 (m, 2H), 4.98 (br d, J=6.4 Hz, 1H), 4.57-4.49 (m, 1H), 4.39 (br s, 2H), 4.03-3.66 (m, 11H), 3.58-3.43 (m, 6H), 3.25-3.08 (m, 3H), 2.79-2.68 (m, 3H), 2.33-2.21 (m, 2H), 2.04-1.45 (m, 21H), 1.33-1.10 (m, 5H).

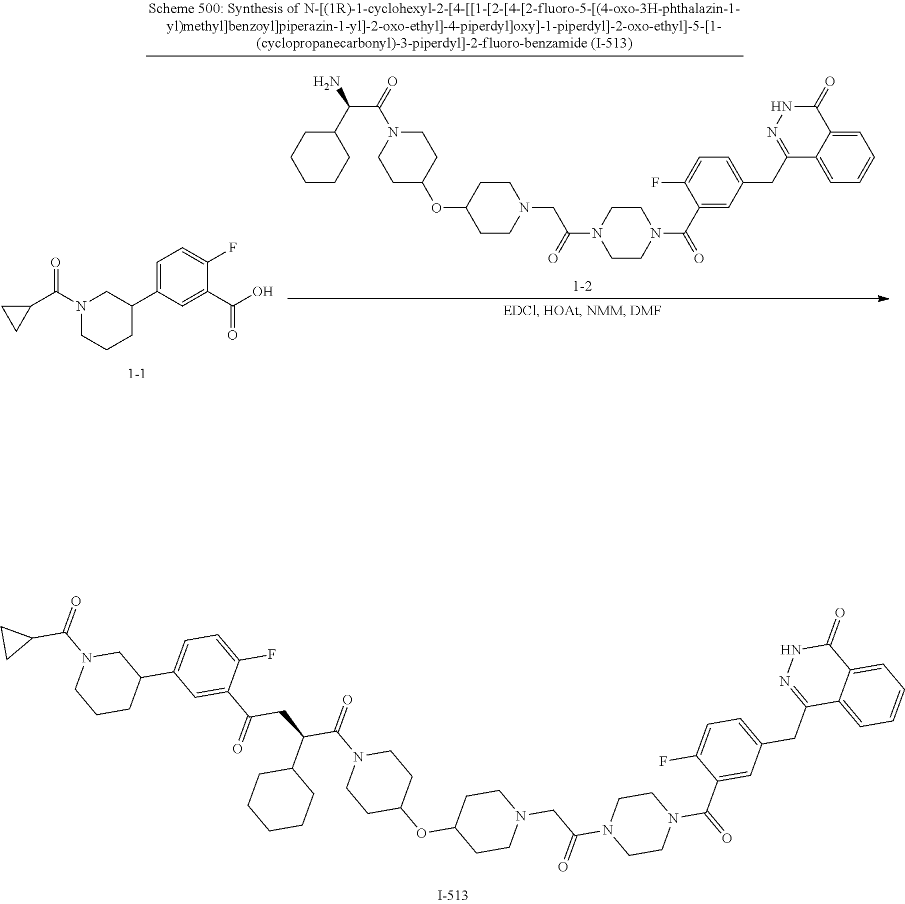 [SMILES: O=C(O)c1cc(C2CCCN(C(=O)C3CC3)C2)ccc1F ; N[C@@H](C(=O)N1CCC(OC2CCN(CC(=O)N3CCN(C(=O)c4cc(Cc5n[nH]c(=O)c6ccccc56)ccc4F)CC3)CC2)CC1)C1CCCCC1 ; O=C(C[C@@H](C(=O)N1CCC(OC2CCN(CC(=O)N3CCN(C(=O)c4cc(Cc5n[nH]c(=O)c6ccccc56)ccc4F)CC3)CC2)CC1)C1CCCCC1)c1cc(C2CCCN(C(=O)C3CC3)C2)ccc1F]

To a solution of Intermediate 1-1 (150 mg, 514.90 μmol, 1 eq) in DMF (3 mL) was added HOAt (70.08 mg, 514.90 μmol, 72.03 μL, 1 eq), EDCI (197.42 mg, 1.03 mmol, 2 eq), NMM (260.40 mg, 2.57 mmol, 283.05 μL, 5 eq) and Intermediate 1-2 (394.59 mg, 514.90 μmol, 1 eq, HCl salt). The mixture was stirred at 25° C. for 1 h. The reaction mixture was quenched by H₂O (6 mL) and extracted with EA (3 mL*3). The combined organic layers were washed with brine (3 mL*3), dried over anhydrous Na₂SO₄, filtered and concentrated under reduced pressure to give a residue. The residue was purified by Prep-HPLC (column: Waters Xbridge 150*25 mm*5 um; mobile phase: [water (ammonia hydroxide v/v)-ACN]; gradient: 30%-60% B over 10 min). I-513 (164.45 mg, 161.79 μmol, 31.42% yield, 98.694% purity) was obtained as a white solid. LCMS (Method G): Rt=0.678 min, (M+H)=1003.7. SFC: Rt=3.079 min, 3.779 min ¹H NMR (400 MHz, METHANOL-d₄) δ=8.37 (br d, J=8.0 Hz, 1H), 7.95 (br d, J=7.6 Hz, 1H), 7.90-7.80 (m, 2H), 7.74-7.62 (m, 1H), 7.48 (br s, 2H), 7.38 (br s, 1H), 7.23-7.11 (m, 2H), 4.98 (br d, J=6.4 Hz, 1H), 4.54 (br d, J=10.4 Hz, 1H), 4.39 (s, 3H), 4.07-3.97 (m, 1H), 3.95-3.42 (m, 12H), 3.19 (br s, 2H), 2.81-2.71 (m, 3H), 2.35-2.21 (m, 2H), 2.04-1.52 (m, 21H), 1.39-1.05 (m, 6H), 0.90-0.74 (m, 4H).

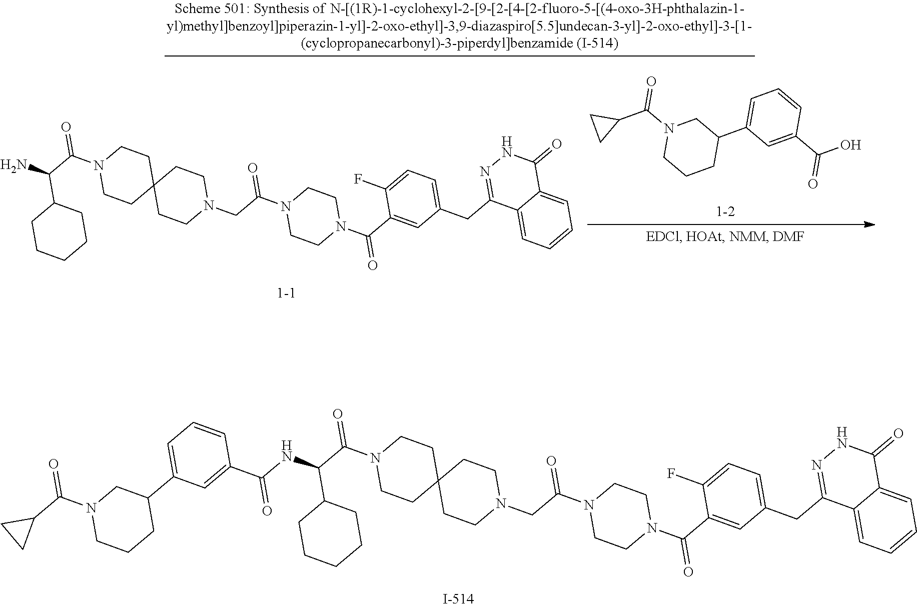

To a solution of Intermediate 1-1 (70 mg, 100.02 μmol, 1 eq), EDCI (38.35 mg, 200.04 μmol, 2 eq), HOAt (13.61 mg, 100.02 μmol, 13.99 μL, 1 eq) and NMM (50.58 mg, 500.10 μmol, 54.98 μL, 5 eq) in DMF (1 mL) was added Intermediate 1-2 (27.34 mg, 100.02 μmol, 1 eq). The mixture was stirred at 25° C. for 2 hr. The mixture was diluted with water (3 ml) and extracted with EtOAc (2 ml*3). The organic layer was concentrated under vacuum. The residue was purified by Prep-HPLC (column: Waters Xbridge 150*25 mm*5 um; mobile phase: [water (NH₄HCO₃)-ACN]; gradient: 40%-70% B over 9 min) and dried by lyophilization. I-514 (31.88 mg, 32.08 μmol, 32.07% yield, 96.12% purity) was obtained as a white solid. LCMS (Method G): Rt=0.661 min, (M+H)=955.7. SFC: Rt=3.938 min, 4.980 min. ¹H NMR (400 MHz, METHANOL-d₄) δ=8.38 (d, J=8.0 Hz, 1H), 7.99-7.94 (m, 1H), 7.90-7.78 (m, 3H), 7.76-7.71 (m, 1H), 7.54-7.46 (m, 2H), 7.46-7.37 (m, 2H), 7.21-7.14 (m, 1H), 4.96 (br d, J=8.0 Hz, 1H), 4.58 (br d, J=12.0 Hz, 1H), 4.39 (s, 3H), 3.84-3.64 (m, 7H), 3.58-3.51 (m, 3H), 3.35 (br s, 1H), 3.31-3.22 (m, 2H), 2.86-2.69 (m, 2H), 2.55 (br s, 4H), 2.10-1.99 (m, 2H), 1.97-1.75 (m, 7H), 1.70 (br d, J=12.0 Hz, 4H), 1.64-1.41 (m, 8H), 1.36-1.21 (m, 3H), 1.17-1.07 (m, 2H), 0.90 (br d, J=4.0 Hz, 2H), 0.86-0.76 (m, 2H).

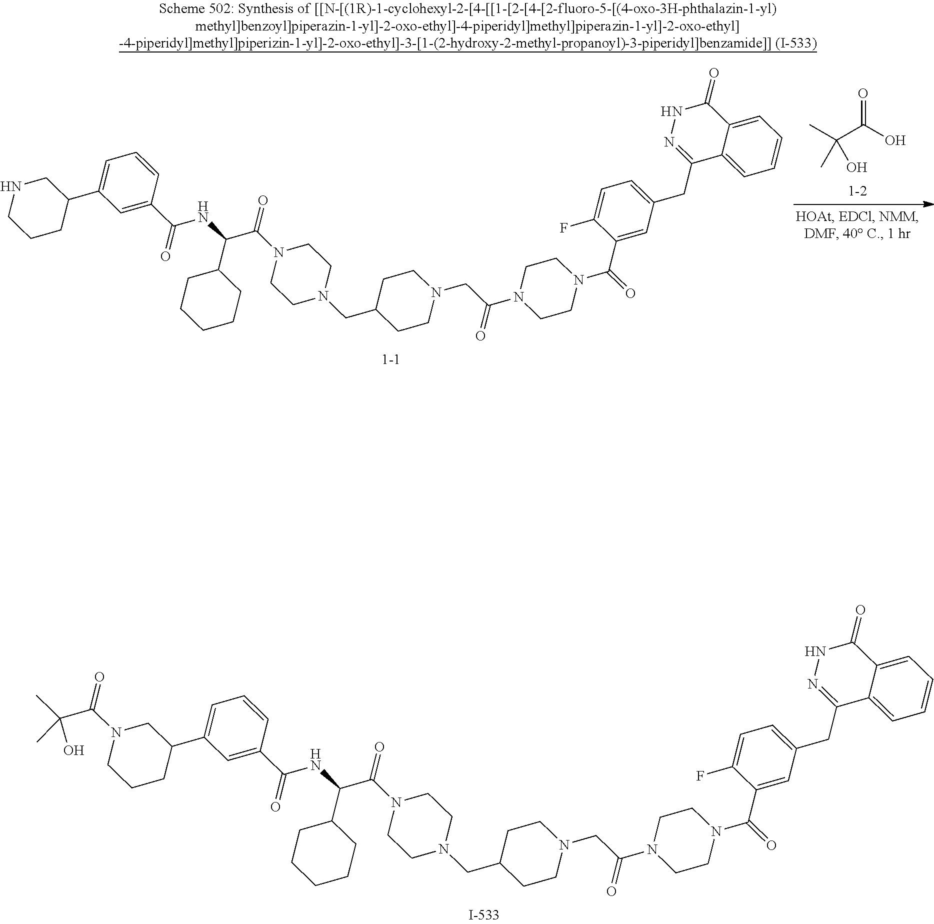

To a solution of intermediate 1-1 (150 mg, 163.73 μmol, 1 eq) and intermediate 1-2 (25.57 mg, 245.60 μmol, 1.5 eq) in DMF (2 mL) was added EDCI (94.16 mg, 491.19 μmol, 3 eq), HOAt (22.29 mg, 163.73 μmol, 22.90 μL, 1 eq), and NMM (82.80 mg, 818.66 μmol, 90.01 μL, 5 eq). The mixture was stirred at 40° C. for 1 hour. The reaction mixture was diluted with water (10 mL) and extracted with DCM (10 mL*2). The combined organic layers were concentrated under reduced pressure to give a residue. The crude product was purified by reversed phase chromatography (0.1% FA condition). The eluent was concentrated under reduced pressure to remove ACN and then lyophilized to give the product. I-533 (59.49 mg, 56.75 μmol, 34.66% yield, 100% purity, FA) was obtained as a yellow solid. LCMS (Method D): Rt: 0.338 min, [M+H]⁺=1002.5. ¹H NMR (400 MHz, METHANOL-d4) δ=8.38 (s, 1H), 8.36 (s, 1H), 7.98-7.92 (m, 1H), 7.91-7.80 (m, 2H), 7.75 (s, 1H), 7.70 (d, J=7.2 Hz, 1H), 7.55-7.34 (m, 4H), 7.19-7.15 (m 1H), 4.89 (d, J=8.8 Hz, 2H), 4.73-4.51 (m, 1H), 4.39 (s, 2H), 4.05-3.97 (m, 1H), 3.94-3.79 (m, 3H), 3.79-3.68 (m, 4H), 3.56 (s, 3H), 3.46-3.32 (m, 5H), 3.19-2.98 (m, 1H), 2.94-2.71 (m, 4H), 2.57-2.41 (m, 4H), 2.30 (d, J=6.0 Hz, 2H), 2.08-1.64 (m, 14H), 1.59-1.36 (m, 8H), 1.33-1.18 (m, 3H), 1.16-1.05 (m, 2H). ¹⁹F NMR (377 MHz, METHANOL-d₄) δ=−120.755.

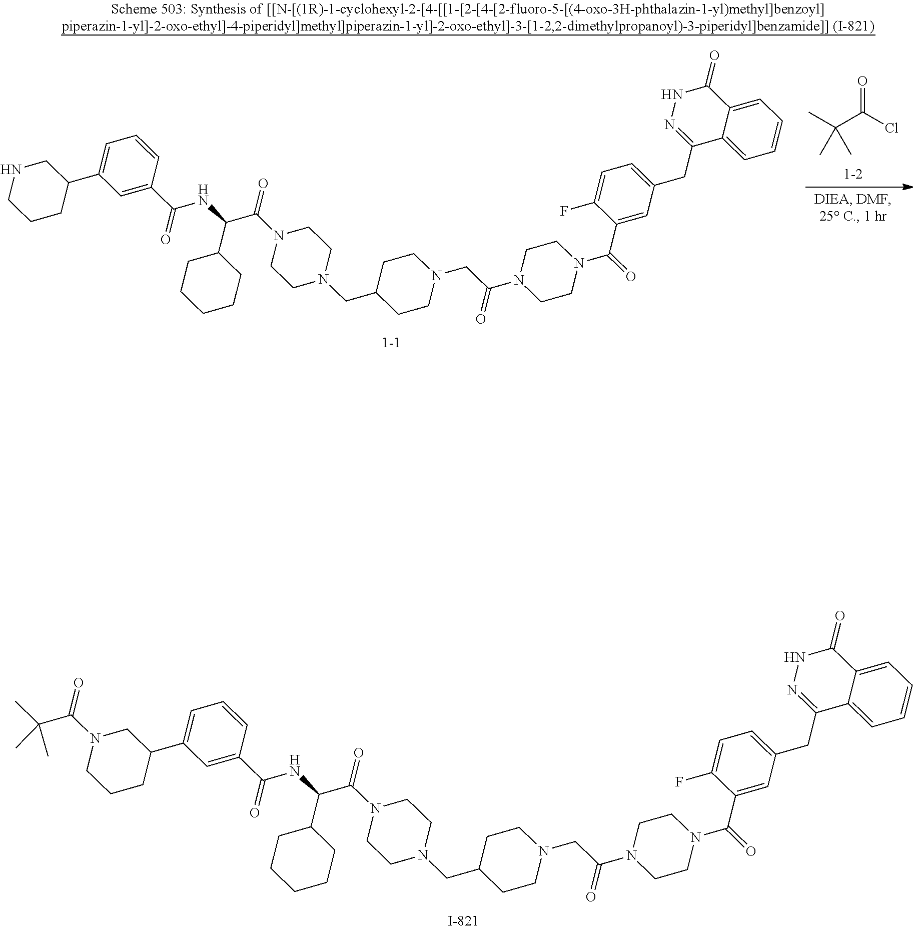

To a solution of Intermediate 1-1 (150 mg, 163.73 μmol, 1 eq) in DMF (1 mL) was added DIEA (63.48 mg, 491.19 μmol, 85.56 μL, 3 eq) and Intermediate 1-2 (29.61 mg, 245.60 μmol, 30.22 μL, 1.5 eq). The mixture was stirred at 25° C. for 1 hr. The reaction mixture was diluted with H₂O (5 mL) and extracted with EA (5 mL*3). The combined organic layers were washed with brine (5 mL*1), dried over Na₂SO₄, filtered and concentrated under reduced pressure to give a residue. The crude product was purified by reversed-phase chromatography (0.1% NH₃·H₂O condition). The eluent was concentrated under reduced pressure to remove ACN and then lyophilized to give product. I-821 (53.28 mg, 53.09 μmol, 32.42% yield, 99.665% purity) was obtained as a white solid. LCMS (Method D): Rt=0.693 min, [M+H]⁺=1000.8. SFC: Rt: 1.952 min, 2.330 min ¹H NMR (400 MHz, METHANOL-d₄) δ=8.41-8.34 (m, 1H), 7.99-7.92 (m, 1H), 7.91-7.80 (m, 2H), 7.77-7.67 (m, 2H), 7.54-7.32 (m, 4H), 7.21-7.12 (m, 1H), 4.94-4.86 (m, 4H), 4.58-4.44 (m, 2H), 4.39 (s, 2H), 3.89-3.64 (m, 7H), 3.60-3.48 (m, 3H), 3.37-3.32 (m, 2H), 3.26-3.20 (m, 1H), 3.06-2.85 (m, 4H), 2.81-2.71 (m, 1H), 2.56-2.33 (m, 4H), 2.26-2.02 (m, 5H), 1.96-1.83 (m, 4H), 1.83-1.73 (m, 4H), 1.72-1.52 (m, 4H), 1.30 (s, 9H), 1.28-1.19 (m, 3H), 1.17-1.04 (m, 2H). ¹⁹FNMR (376 MHz, MeOD-d₆) δ=−120.809.

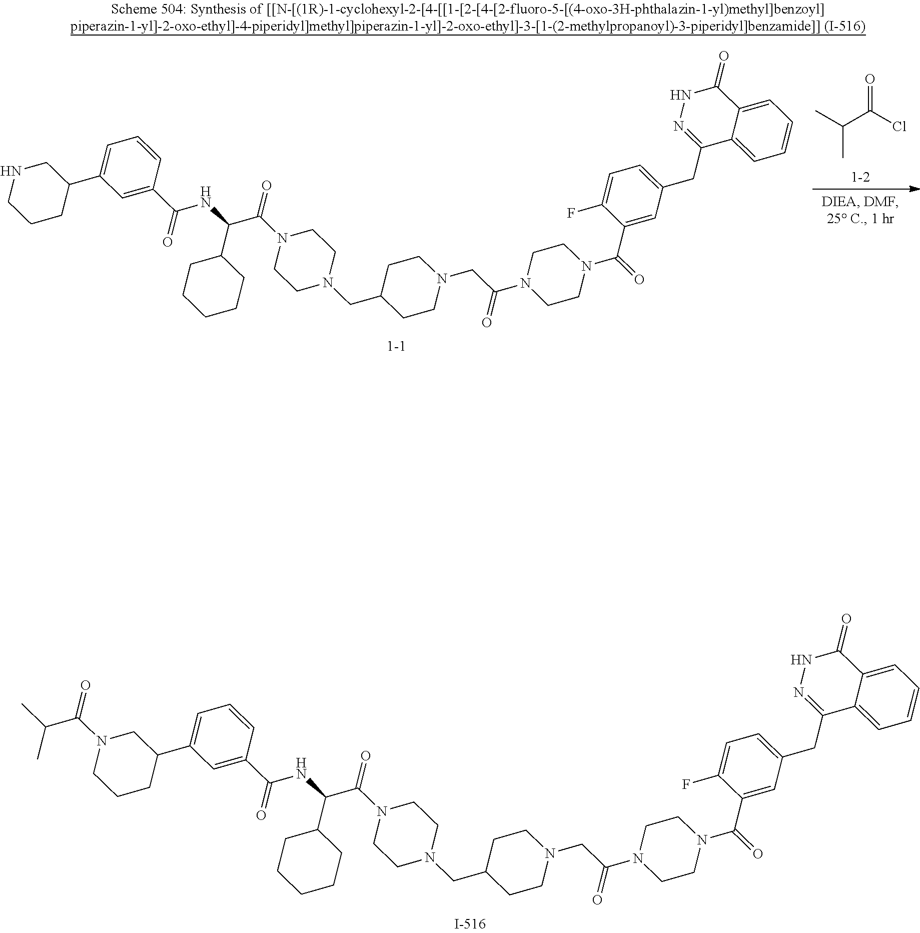

To a solution of Intermediate 1-1 (250 mg, 272.89 μmol, 1 eq) in DMF (2 mL) was added DIPEA (105.81 mg, 818.66 μmol, 142.59 μL, 3 eq) and Intermediate 1-2 (43.61 mg, 409.33 μmol, 42.89 μL, 1.5 eq). The mixture was stirred at 25° C. for 1 hr. The reaction mixture was purified by reversed-phase chromatography (0.1% FA condition). The eluent was concentrated under reduced pressure to remove ACN and then lyophilized to give product. I-516 (108.08 mg, 109.04 μmol, 39.96% yield, 99.5% purity) was obtained as a white solid. LCMS (Method D): Rt: 0.354 min, [M+H]⁺=986.7. ¹H NMR (400 MHz, METHANOL-d4) δ=8.37 (d, J=7.2 Hz, 1H), 7.98-7.92 (m, 1H), 7.91-7.80 (m, 2H), 7.76 (s, 1H), 7.74-7.67 (m, 1H), 7.53-7.41 (m, 3H), 7.38 (s, 1H), 7.20-7.12 (m, 1H), 4.93-4.88 (m, 1H), 4.66-4.57 (m, 1H), 4.38 (s, 2H), 4.15-4.03 (m, 1H), 3.89-3.63 (m, 7H), 3.61-3.47 (m, 3H), 3.38-3.32 (m, 1H), 3.30-3.13 (m, 4H), 3.06-2.61 (m, 5H), 2.56-2.33 (m, 4H), 2.27-2.15 (m, 2H), 2.13-2.01 (m, 3H), 1.96-1.82 (m, 4H), 1.82-1.51 (m, 8H), 1.35-1.17 (m, 5H), 1.17-1.03 (m, 8H). SFC: Rt: 1.989 min, 2.261 min ¹⁹F NMR (376 MHz, METHANOL-d4) δ=−120.779.

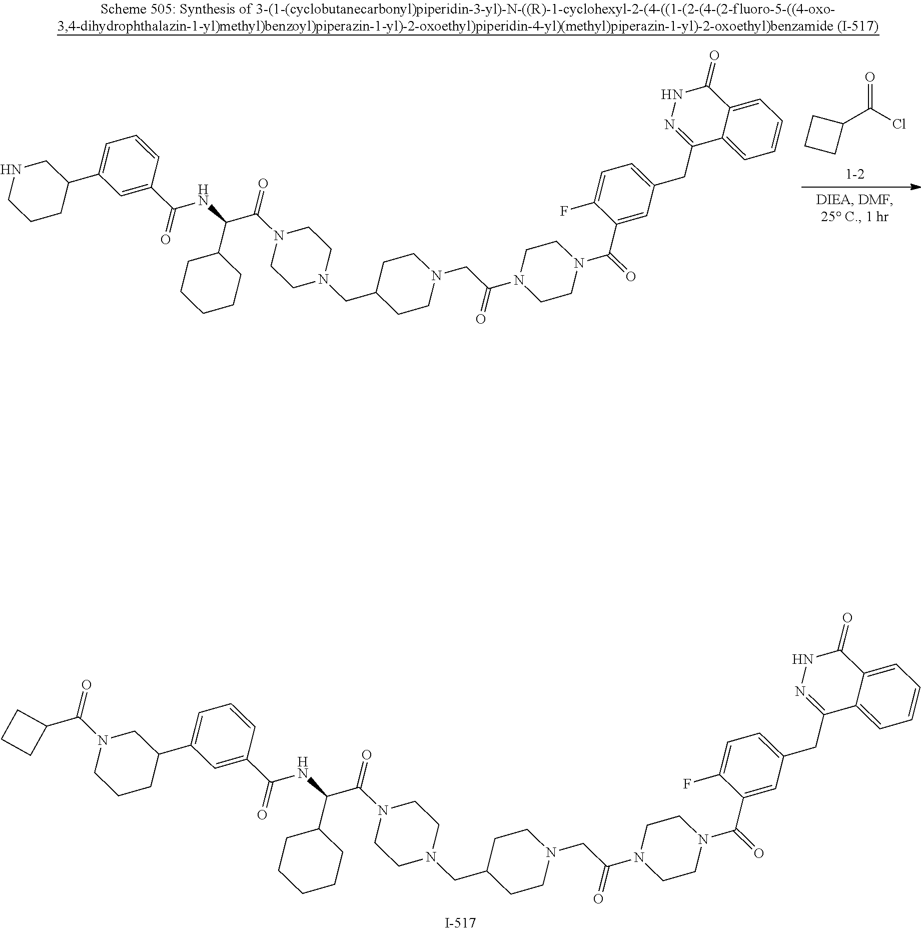

To a solution of Intermediate 1-1 (200 mg, 218.31 μmol, 1 eq) in DMF (2 mL) was added DIEA (84.64 mg, 654.93 μmol, 114.08 μL, 3 eq) and Intermediate 1-2 (38.82 mg, 327.46 μmol, 37.37 μL, 1.5 eq). The mixture was stirred at 25° C. for 1 hr. The mixture was diluted with water (10 mL) and extracted with EA (10 mL*3). The combined organic layers were dried over sodium sulfate, filtered and concentrated under reduced pressure to give a residue. The residue was purified by reversed phase chromatography (NH₃·H₂O condition), the eluent was concentrated under reduced pressure to remove ACN and lyophilized to give I-517 (70.07 mg, 69.95 μmol, 32.04% yield, 99.657% purity) as a white solid. LCMS (Method D): Rt=0.346 min, [M+H]⁺=998.5. SFC: Rt: 3.009 min, 3.735 min. ¹H NMR (400 MHz, METHANOL-d4) δ=8.37 (d, J=7.6 Hz, 1H), 7.98-7.92 (m, 1H), 7.90-7.81 (m, 2H), 7.78-7.68 (m, 2H), 7.51-7.37 (m, 4H), 7.16 (m, 1H), 4.91 (s, 1H), 4.57 (s, 1H), 4.39 (s, 2H), 3.89-3.78 (m, 3H), 3.77-3.66 (m, 5H), 3.58-3.49 (m, 3H), 3.48-3.41 (m, 1H), 3.28 (s, 1H), 3.24-3.19 (m, 1H), 3.15-3.05 (m, 1H), 2.97-2.87 (m, 2H), 2.82-2.63 (m, 2H), 2.55-2.43 (m, 3H), 2.38 (s, 1H), 2.36-2.27 (m, 2H), 2.26-2.17 (m, 4H), 2.12-1.99 (m, 4H), 1.92-1.72 (m, 10H), 1.71-1.65 (m, 2H), 1.61-1.51 (m, 2H), 1.37-1.17 (m, 6H), 1.15-1.04 (m, 2H). ¹⁹F NMR (377 MHz, METHANOL-d4) δ=−111.64-−135.60.

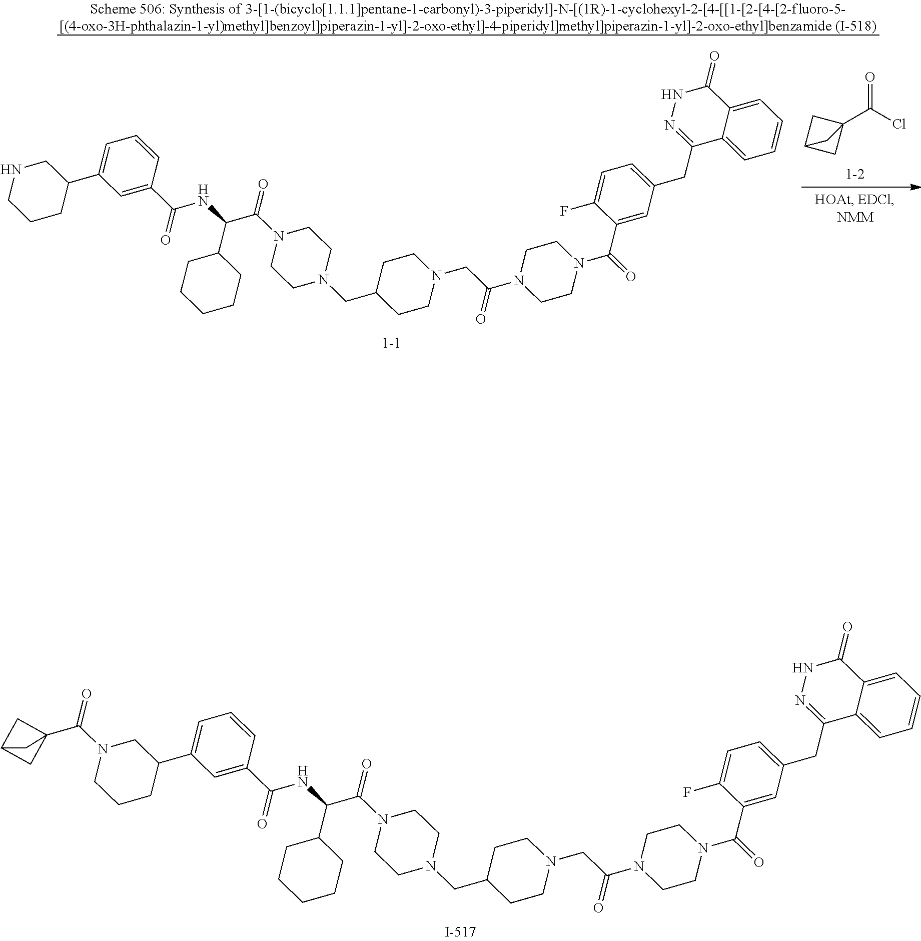

To a solution of Intermediate 1-1 (100.00 mg, 109.15 μmol, 1 eq), Intermediate 1-2 (12.24 mg, 109.15 μmol, 1 eq) and HOAt (14.86 mg, 109.15 μmol, 15.27 μL, 1 eq) in DMF (1 mL) was added EDCI (41.85 mg, 218.31 μmol, 2 eq) and NMM (55.20 mg, 545.77 μmol, 60.00 μL, 5 eq). The mixture was stirred at 25° C. for 2 hrs. The mixture was diluted with water (10 mL) and extracted with EtOAc (10 mL*3). The organic layer was dried with anhydrous Na₂SO₄, filtered and concentrated under vacuum. The residue was purified by Prep-HPLC (column: Waters Xbridge 150*25 mm*5 um; mobile phase: [water (NH₄HCO₃)-ACN]; gradient: 40%-70% B over 9 min) and dried by lyophilization. I-518 (65.72 mg, 57.69 μmol, 52.86% yield, 98.698% purity, TFA salt) was obtained as an off-white solid. LCMS: Rt=1.993 min, [M+H]⁺=1010.9. SFC: Rt=4.307 min, 4.916 min. ¹H NMR (400 MHz, METHANOL-d₄) δ=8.37 (d, J=7.6 Hz, 1H), 7.98-7.92 (m, 1H), 7.91-7.80 (m, 2H), 7.78-7.66 (m, 2H), 7.53-7.41 (m, 3H), 7.41-7.34 (m, 1H), 7.21-7.12 (m, 1H), 4.92-4.88 (m, 1H), 4.56-4.46 (m, 1H), 4.39 (s, 2H), 4.32-4.30 (m, 1H), 3.89-3.65 (m, 7H), 3.60-3.45 (m, 4H), 3.42-3.33 (m, 2H), 3.22-2.97 (m, 3H), 2.79-2.63 (m, 2H), 2.55-2.42 (m, 4H), 2.39 (br s, 2H), 2.27-2.10 (m, 9H), 2.08-2.01 (m, 1H), 1.94-1.74 (m, 8H), 1.72-1.56 (m, 4H), 1.40-1.18 (m, 6H), 1.17-1.02 (m, 2H). ¹⁹F NMR (377 MHz, METHANOL-d₄) δ=−76.890, −120.762.

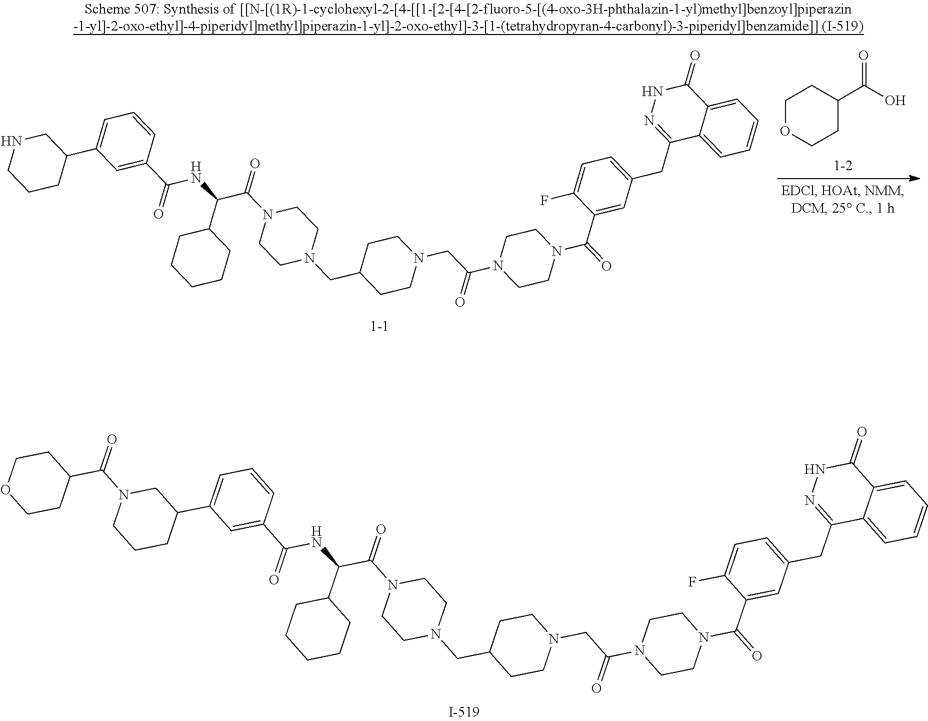

To a solution of Intermediate 1-2 (14.21 mg, 109.15 μmol, 1 eq) in DCM (1.5 mL) was added NMM (55.20 mg, 545.77 μmol, 60.00 μL, 5 eq), EDCI (62.78 mg, 327.46 μmol, 3 eq) and HOAt (14.86 mg, 109.15 μmol, 15.27 μL, 1 eq) at 25° C. for 0.5 hr. And then Intermediate 1-1 (100 mg, 109.15 μmol, 1 eq) was added into the mixture and it was stirred at 25° C. for 0.5 hr. The reaction mixture was diluted with water (5 mL) and extracted with DCM (5 mL×3). The combined organic layers were washed with brine (5 mL×3) and dried over sodium sulfate, filtered and concentrated under reduced pressure to give a residue. The crude product was purified by reversed phase chromatography (0.1% NH₃·H₂O condition) and the eluent was concentrated under reduced pressure to remove ACN and then lyophilized to give I-519 (46.38 mg, 45.11 μmol, 41.32% yield, 100% purity) as a white solid. LCMS (Method D): Rt: 0.340 min, [M+H]⁺=1028.5. ¹H NMR (400 MHz, METHANOL-d4) δ=8.37 (d, J=8.0 Hz, 1H), 7.99-7.92 (m, 1H), 7.87-7.82 (m, 2H), 7.79-7.66 (m, 2H), 7.54-7.34 (m, 4H), 7.16 (s, 1H), 4.95-4.88 (m, 1H), 4.59 (bd, J=10.4 Hz, 1H), 4.38 (s, 2H), 4.10-4.08 (m, 1H), 4.02-3.88 (m, 2H), 3.86-3.64 (m, 7H), 3.59-3.34 (m, 6H), 3.27-3.12 (m, 3H), 3.06-2.85 (m, 3H), 2.83-2.65 (m, 2H), 2.56-2.34 (m, 4H), 2.21-2.20 (m, 2H), 2.13-2.01 (m, 3H), 1.98-1.48 (m, 17H), 1.34-1.17 (m, 5H), 1.15-1.03 (m, 2H). ¹⁹F NMR (376 MHz, METHANOL-d4) δ=−120.802. SFC: Rt: 2.848 min, 3.326 min.

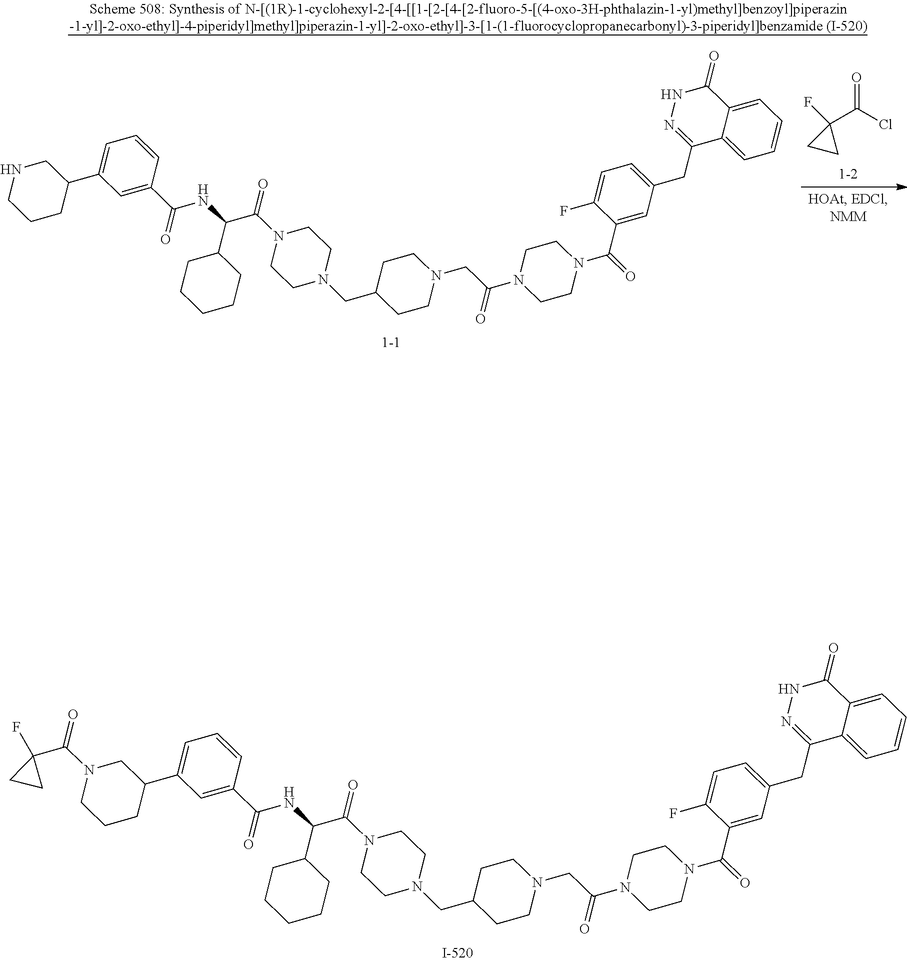

To a solution of Intermediate 1-1 (100.00 mg, 104.98 μmol, 1 eq, HCl salt) in DMF (1 mL) was added Intermediate 1-2 (10.93 mg, 104.98 μmol, 1 eq), EDCI (40.25 mg, 209.95 μmol, 2 eq), NMM (53.09 mg, 524.88 μmol, 57.71 μL, 5 eq) and HOAt (14.29 mg, 104.98 μmol, 14.68 μL, 1 eq). The mixture was stirred at 25° C. for 1 hr. The mixture was diluted with water (5 mL) and extracted with EtOAc (10 mL*3). The organic layer was concentrated under vacuum. The residue was purified by Prep-HPLC (column: Waters Xbridge 150*25 mm*5 um; mobile phase: [water (NH₄HCO₃)-ACN]; gradient: 42%-72% B over 9 min) and dried by lyophilization. I-520 (47.15 mg, 45.47 μmol, 43.31% yield, 96.64% purity) was obtained as a white solid. LCMS (Method G): Rt=0.679 min, [M+H]⁺=1002.7. SFC: Rt=2.643 min, 3.033 min. ¹H NMR (400 MHz, METHANOL-d₄) δ=8.39-8.35 (m, 1H), 7.97-7.93 (m, 1H), 7.90-7.85 (m, 1H), 7.85-7.81 (m, 1H), 7.76 (s, 1H), 7.71 (d, J=7.6 Hz, 1H), 7.51-7.46 (m, 2H), 7.46-7.41 (m, 1H), 7.38 (br s, 1H), 7.19-7.15 (m, 1H), 4.90 (br d, J=9.2 Hz, 1H), 4.51-4.36 (m, 4H), 3.88-3.77 (m, 2H), 3.76-3.63 (m, 5H), 3.59-3.49 (m, 3H), 3.29-3.12 (m, 4H), 2.96-2.75 (m, 4H), 2.52-2.33 (m, 4H), 2.26-2.17 (m, 2H), 2.13-2.02 (m, 3H), 1.96-1.84 (m, 4H), 1.82-1.73 (m, 4H), 1.69 (br d, J=11.6 Hz, 3H), 1.61-1.52 (m, 1H), 1.40-1.14 (m, 10H), 1.14-1.05 (m, 2H). ¹⁹F NMR (377 MHz, METHANOL-d₄) δ=−120.720, −187.592.

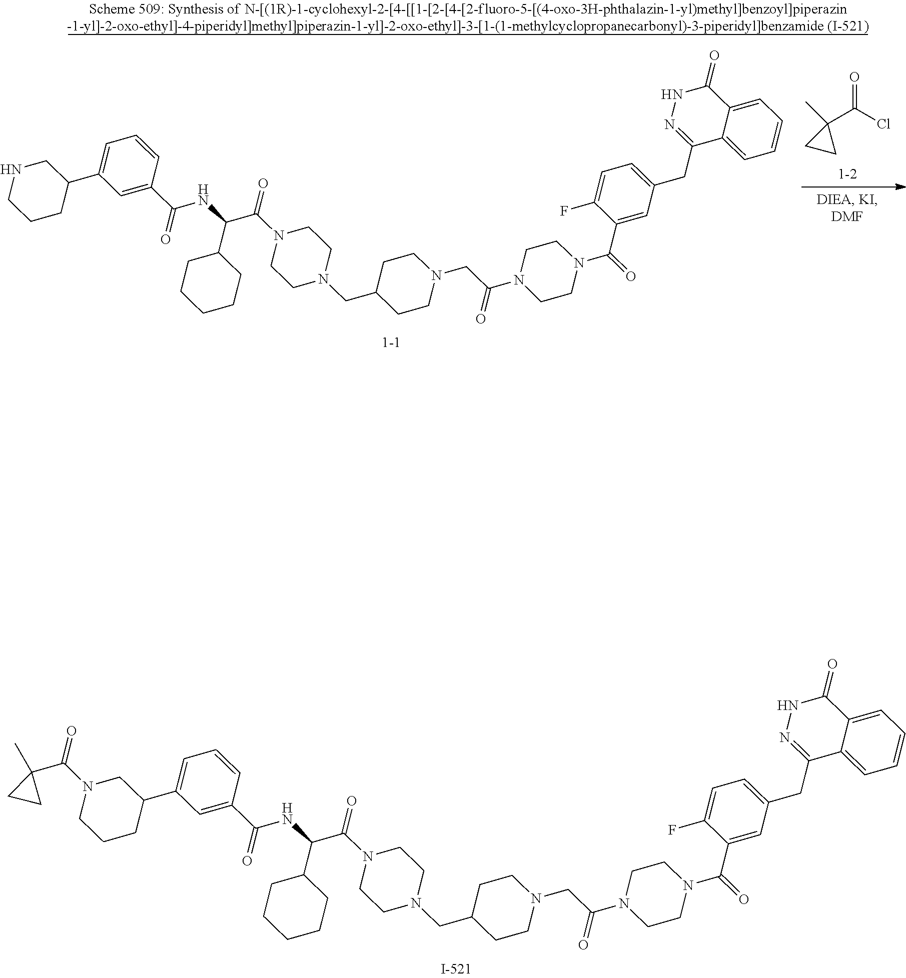

To a solution of Intermediate 1-1 (150.00 mg, 157.46 μmol, 1 eq) in DMF (1.5 mL) was added Intermediate 1-2 (18.67 mg, 157.46 μmol, 1 eq), DIEA (61.05 mg, 472.39 μmol, 82.28 μL, 3 eq) and KI (78.42 mg, 472.39 μmol, 3 eq). The mixture was stirred at 25° C. for 1 hr. The mixture was diluted with water (5 mL) and extracted with EtOAc (10 mL*3). The organic layer was concentrated under vacuum. The residue was purified by Prep-HPLC (column: Waters Xbridge 150*25 mm*5 um; mobile phase: [water (NH₄HCO₃)-ACN]; gradient: 32%-62% B over 15 min) and dried by lyophilization. I-521 (43.27 mg, 43.35 μmol, 27.53% yield, 100% purity) was obtained as a white solid. LCMS (Method G): Rt=0.659 min, [M+H]⁺=998.7. SFC: Rt=3.396 min, 4.063 min. ¹H NMR (400 MHz, METHANOL-d₄) δ=8.37 (d, J=7.6 Hz, 1H), 7.97-7.93 (m, 1H), 7.88-7.83 (m, 2H), 7.76 (s, 1H), 7.71 (br d, J=7.6 Hz, 1H), 7.52-7.47 (m, 2H), 7.46-7.41 (m, 1H), 7.38 (br s, 1H), 7.19-7.15 (m, 1H), 4.92-4.89 (m, 1H), 4.53-4.44 (m, 2H), 4.39 (s, 2H), 3.85-3.65 (m, 7H), 3.63-3.37 (m, 4H), 3.27-3.12 (m, 3H), 2.97-2.72 (m, 4H), 2.52-2.33 (m, 4H), 2.25-2.20 (m, 2H), 2.14-2.02 (m, 3H), 1.96-1.71 (m, 9H), 1.71-1.51 (m, 4H), 1.32 (s, 4H), 1.30-1.20 (m, 4H), 1.20-1.11 (m, 2H), 1.11-1.04 (m, 1H), 0.95-0.90 (m, 2H), 0.63 (s, 1H). ¹⁹F NMR (377 MHz, METHANOL-d₄) δ=−120.803.

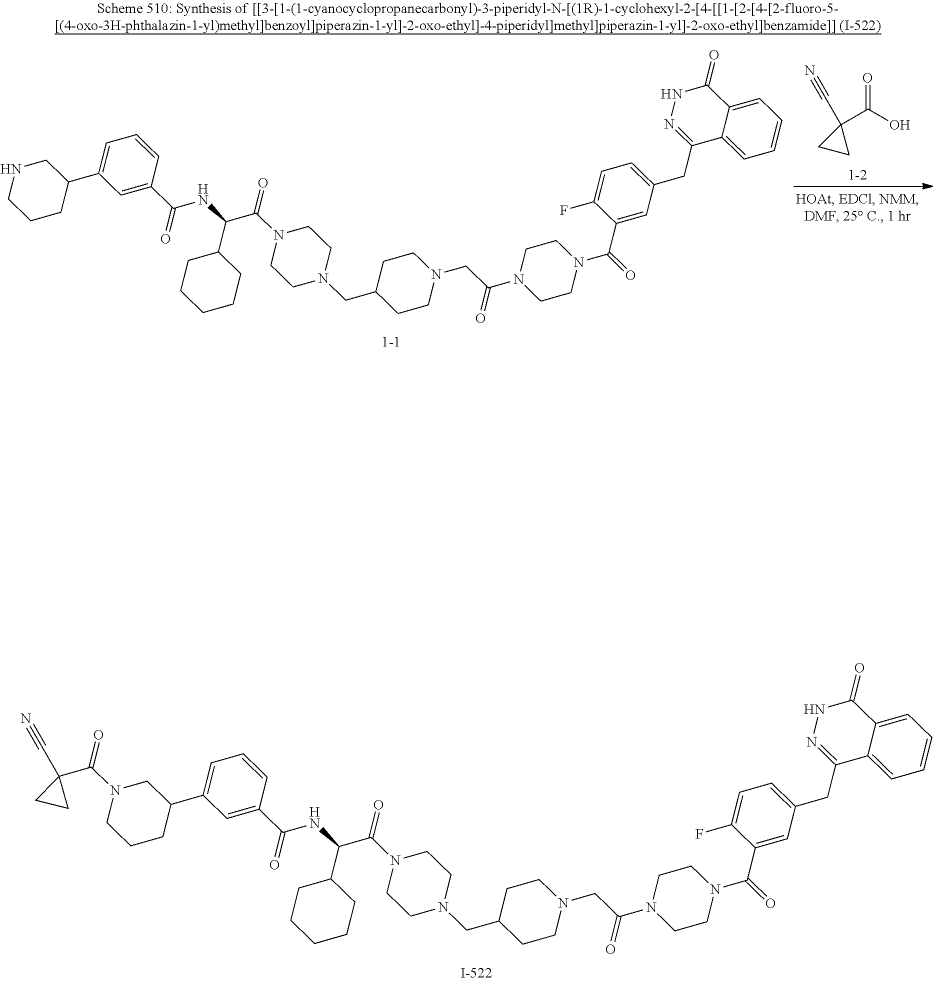

To a solution of intermediate 1-2 (13.34 mg, 120.07 μmol, 1.1 eq) in DMF (0.5 mL) was added intermediate 1-1 (100 mg, 109.15 μmol, 1 eq), HOAT (14.86 mg, 109.15 μmol, 15.27 μL, 1 eq), EDCI (62.78 mg, 327.46 μmol, 3 eq) and NMM (55.20 mg, 545.77 μmol, 60.00 μL, 5 eq). The mixture was stirred at 25° C. for 1 hr. The reaction mixture was diluted with H₂O (2 mL) and extracted with EA (2 mL*3). The combined organic layers were washed by brine (2 mL*3), dried over Na₂SO₄, filtered and concentrated under reduced pressure to give a residue. The residue was purified by reversed-phase chromatography (0.1% NH₃·H₂O condition). The eluent was concentrated and lyophilized to give I-522 (100.95 mg, 99.22 μmol, 90.90% yield, 99.193% purity) as a white solid. LCMS (Method D): Retention time: 0.345 min, [M+H]⁺=1009.5. SFC: Retention time: 2.789, 3.032 min. ¹H NMR (400 MHz, METHANOL-d4) δ=8.37 (d, J=7.2 Hz, 1H), 7.98-7.92 (m, 1H), 7.91-7.78 (m, 3H), 7.72 (d, J=7.2 Hz, 1H), 7.54-7.42 (m, 3H), 7.38 (s, 1H), 7.18-7.14 (m, 1H), 4.90 (d, J=8.4 Hz, 1H), 4.49-4.42 (m, 2H), 4.39 (s, 2H), 3.92-3.60 (m, 8H), 3.59-3.50 (m, 3H), 3.37-3.33 (m, 1H), 3.22 (s, 1H), 2.99-2.85 (m, 3H), 2.52-2.35 (m, 4H), 2.24-2.19 (m, 2H), 2.09 (d, J=11.6 Hz, 3H), 2.01-1.83 (m, 5H), 1.77 (s, 5H), 1.69 (d, J=12.4 Hz, 3H), 1.57 (s, 5H), 1.37-1.17 (m, 6H), 1.16-1.05 (m, 2H). ¹⁹F NMR (377 MHz, CHLOROFORM-d) δ=−120.79.

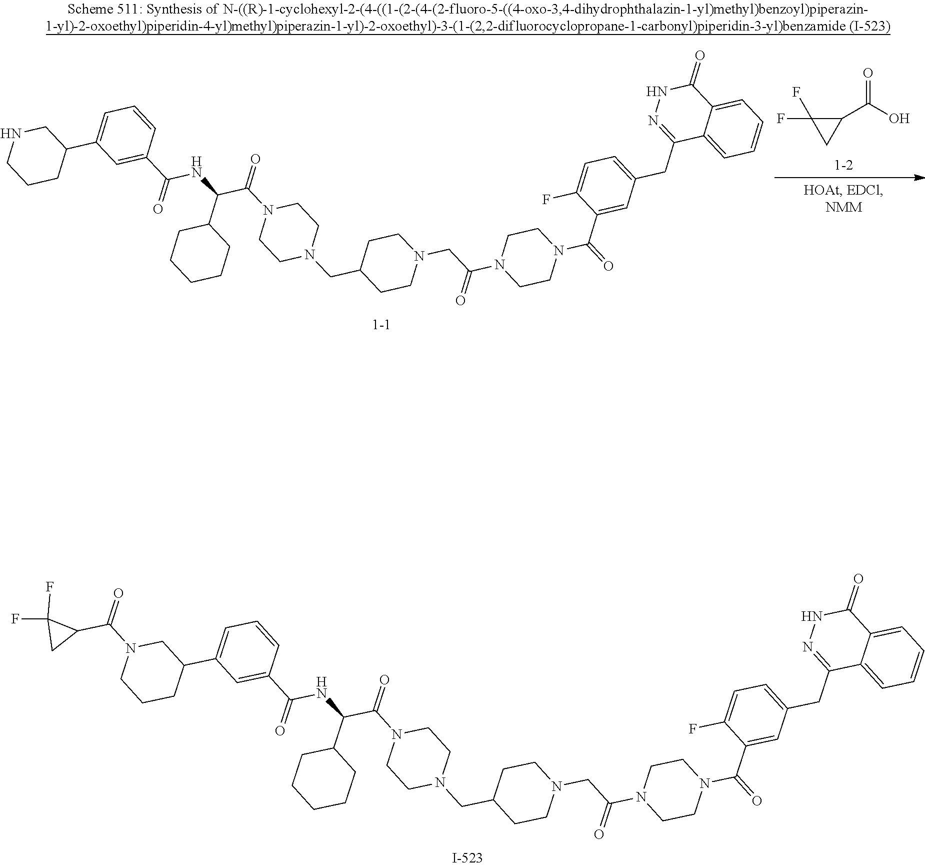

To a solution of Intermediate 1-1 (100 mg, 109.15 μmol, 1 eq) and Intermediate 1-2 (19.99 mg, 163.73 μmol, 1.5 eq) in DMF (1 mL) was added EDCI (104.63 mg, 545.77 μmol, 5 eq), NMM (110.41 mg, 1.09 mmol, 120.01 μL, 10 eq) and HOAt (44.57 mg, 327.46 μmol, 45.81 μL, 3 eq). The mixture was stirred at 25° C. for 0.5 hr. The mixture was directly purified by Prep-HPLC (column: Waters Xbridge 150*25 mm*5 um; mobile phase: [water (NH₄HCO₃)-ACN]; gradient: 32%-62% B over 15 min). The eluent was concentrated to remove MeCN and then lyophilized to afford the product. I-523 (49.05 mg, 47.60 μmol, 43.61% yield, 99% purity) was obtained as a white solid. LCMS (Method G): Rt=0.685 min, M+H=1020.6. SFC: Rt=1.041 min, 1.132 min, 1.238 min, 1.599 min. ¹H NMR (400 MHz, METHANOL-d₄) δ =8.40-8.35 (m, 1H), 7.98-7.93 (m, 1H), 7.91-7.80 (m, 2H), 7.79-7.68 (m, 2H), 7.55-7.35 (m, 4H), 7.20-7.12 (m, 1H), 4.92-4.88 (m, 1H), 4.61-4.54 (m, 1H), 4.41-4.36 (m, 2H), 4.23-4.12 (m, 1H), 3.89-3.64 (m, 7H), 3.59-3.48 (m, 3H), 3.27-3.16 (m, 3H), 3.15-2.64 (m, 6H), 2.55-2.33 (m, 4H), 2.24-2.19 (m, 2H), 2.13-1.85 (m, 8H), 1.81-1.53 (m, 9H), 1.40-1.01 (m, 8H).

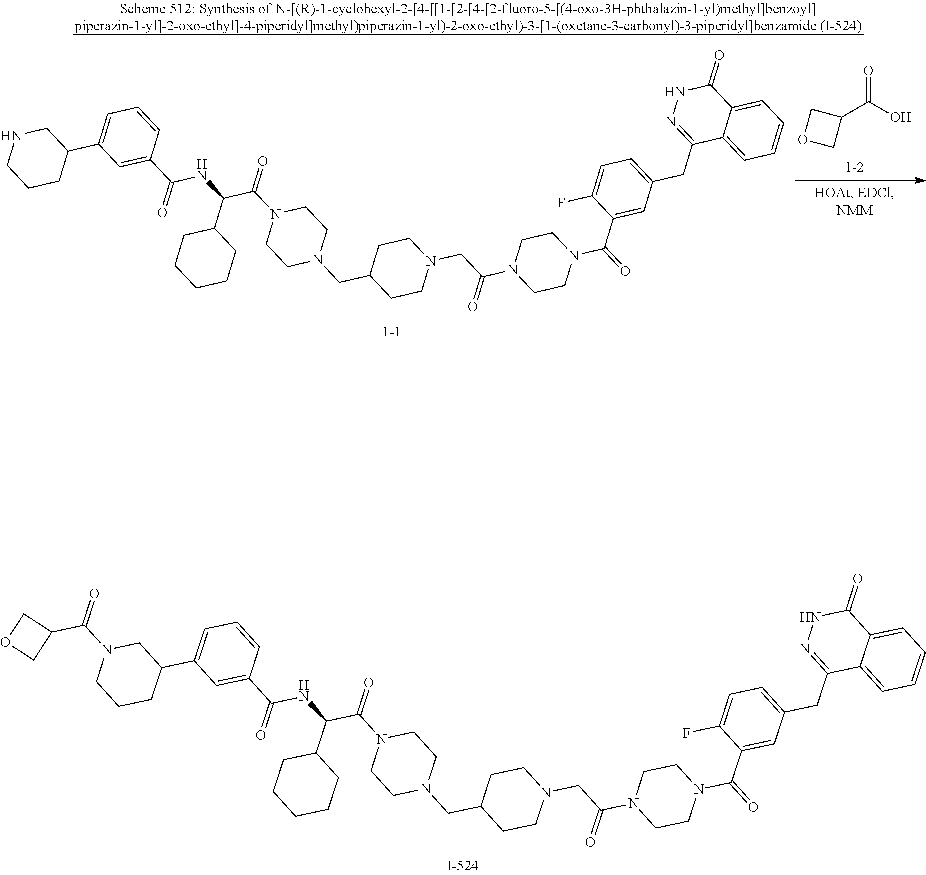

To a solution of Intermediate 1-1 (100.00 mg, 104.98 μmol, 1 eq) in DMF (1 mL) was added Intermediate 1-2 (10.72 mg, 104.98 μmol, 1 eq), EDCI (40.25 mg, 209.95 μmol, 2 eq), NMM (53.09 mg, 524.88 μmol, 57.71 μL, 5 eq) and HOAt (14.29 mg, 104.98 μmol, 14.68 μL, 1 eq). The mixture was stirred at 25° C. for 1 hr. The mixture was diluted with water (5 mL) and extracted with EtOAc (10 mL*3). The organic layer was concentrated under vacuum. The residue was purified by Prep-HPLC (column: Waters Xbridge 150*25 mm*5 um; mobile phase: [water (NH₄HCO₃)-ACN]; gradient: 25%-55% B over 15 min) and dried by lyophilization. I-524 (38.69 mg, 38.68 μmol, 36.85% yield, 100% purity) was obtained as a white solid. LCMS (Method G): Rt=0.614 min, [M+H]⁺=1000.7. SFC: Rt=3.318 min, 3.952 min. ¹H NMR (400 MHz, METHANOL-d₄) δ=8.39-8.35 (m, 1H), 7.95 (br d, J=8.4 Hz, 1H), 7.90-7.86 (m, 1H), 7.85-7.81 (m, 1H), 7.77-7.69 (m, 2H), 7.51-7.46 (m, 2H), 7.46-7.41 (m, 1H), 7.40-7.35 (m, 1H), 7.19-7.13 (m, 1H), 4.92-4.88 (m, 3H), 4.84-4.82 (m, 1H), 4.81-4.76 (m, 1H), 4.64-4.57 (m, 1H), 4.39 (s, 2H), 4.26-4.19 (m, 1H), 3.87-3.78 (m, 2H), 3.76-3.65 (m, 5H), 3.62-3.43 (m, 5H), 3.27-3.18 (m, 2H), 3.16-3.05 (m, 1H), 2.96-2.86 (m, 2H), 2.82-2.69 (m, 2H), 2.52-2.34 (m, 4H), 2.49-2.20 (m, 2H), 2.12-2.03 (m, 3H), 1.98-1.84 (m, 4H), 1.82-1.75 (m, 4H), 1.72-1.66 (m, 2H), 1.63-1.52 (m, 2H), 1.33-1.05 (m, 8H). ¹⁹F NMR (377 MHz, METHANOL-d₄) δ=−120.809.

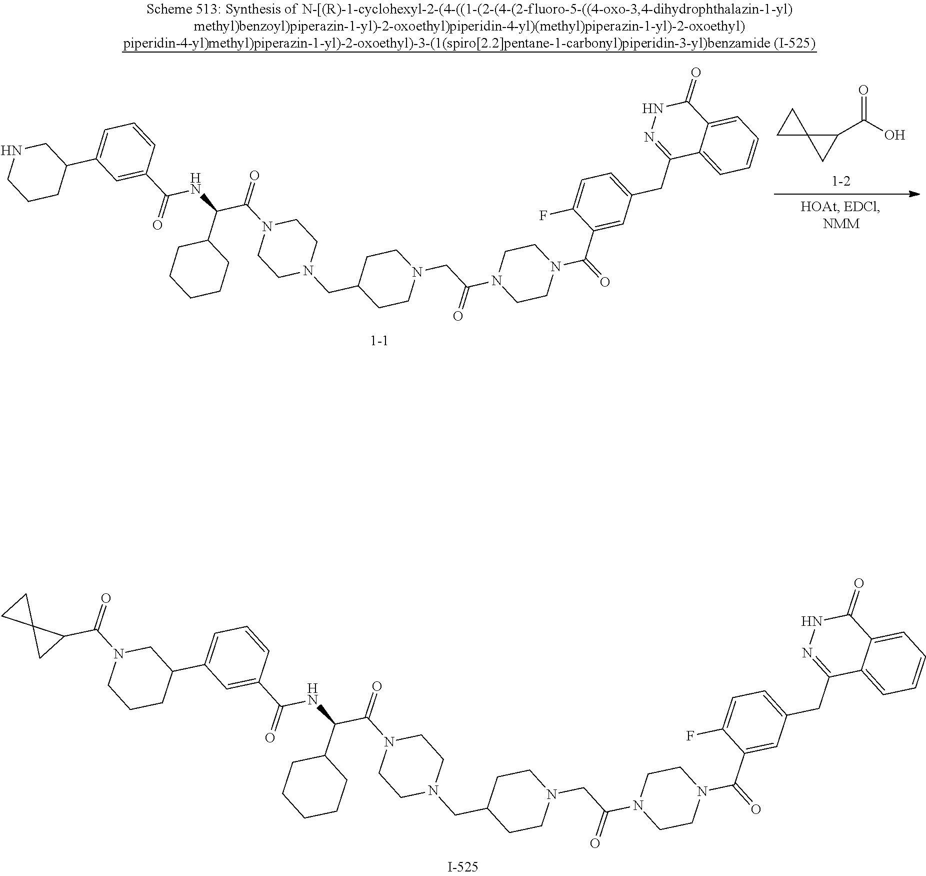

To a solution of Intermediate 1-1 (100 mg, 109.15 μmol, 1 eq) and Intermediate 1-2 (18.36 mg, 163.73 μmol, 1.5 eq) in DMF (0.8 mL) was added EDCI (104.63 mg, 545.77 μmol, 5 eq), NMM (110.41 mg, 1.09 mmol, 120.01 μL, 10 eq) and HOAt (44.57 mg, 327.46 μmol, 45.81 μL, 3 eq). The mixture was stirred at 25° C. for 0.5 hr. The reaction mixture was partitioned between EtOAc (10 mL) and water (3 mL). Then the organic layers were dried over anhydrous Na₂SO₄ and concentrated under reduced pressure to give a residue. The residue was purified by Prep-HPLC (column: Waters Xbridge 150*25 mm*5 um; mobile phase: [water (NH₄HCO₃)-ACN]; gradient: 32%-62% B over 15 min). The eluent was concentrated to remove MeCN and then lyophilized to afford the product. I-525 (41.07 mg, 40.25 mol, 36.87% yield, 99% purity) was obtained as a white solid. LCMS (Method G): Rt=0.695 min, M+H=1010.6. SFC: Rt=4.556 min, 5.425 min, 5.695 min. ¹H NMR (400 MHz, METHANOL-d₄) δ=8.40-8.34 (m, 1H), 7.98-7.93 (m, 1H), 7.91-7.81 (m, 2H), 7.78-7.67 (m, 2H), 7.52-7.35 (m, 4H), 7.20-7.12 (m, 1H), 4.92-4.89 (m, 1H), 4.61-4.53 (m, 1H), 4.41-4.36 (m, 2H), 4.19-4.08 (m, 1H), 3.89-3.65 (m, 7H), 3.59-3.49 (m, 3H), 3.28-3.08 (m, 4H), 2.96-2.86 (m, 2H), 2.84-2.63 (m, 2H), 2.55-2.32 (m, 5H), 2.26-2.17 (m, 2H), 2.13-2.01 (m, 3H), 1.96-1.60 (m, 11H), 1.59-1.49 (m, 2H), 1.39-0.99 (m, 9H), 0.98-0.70 (m, 4H).

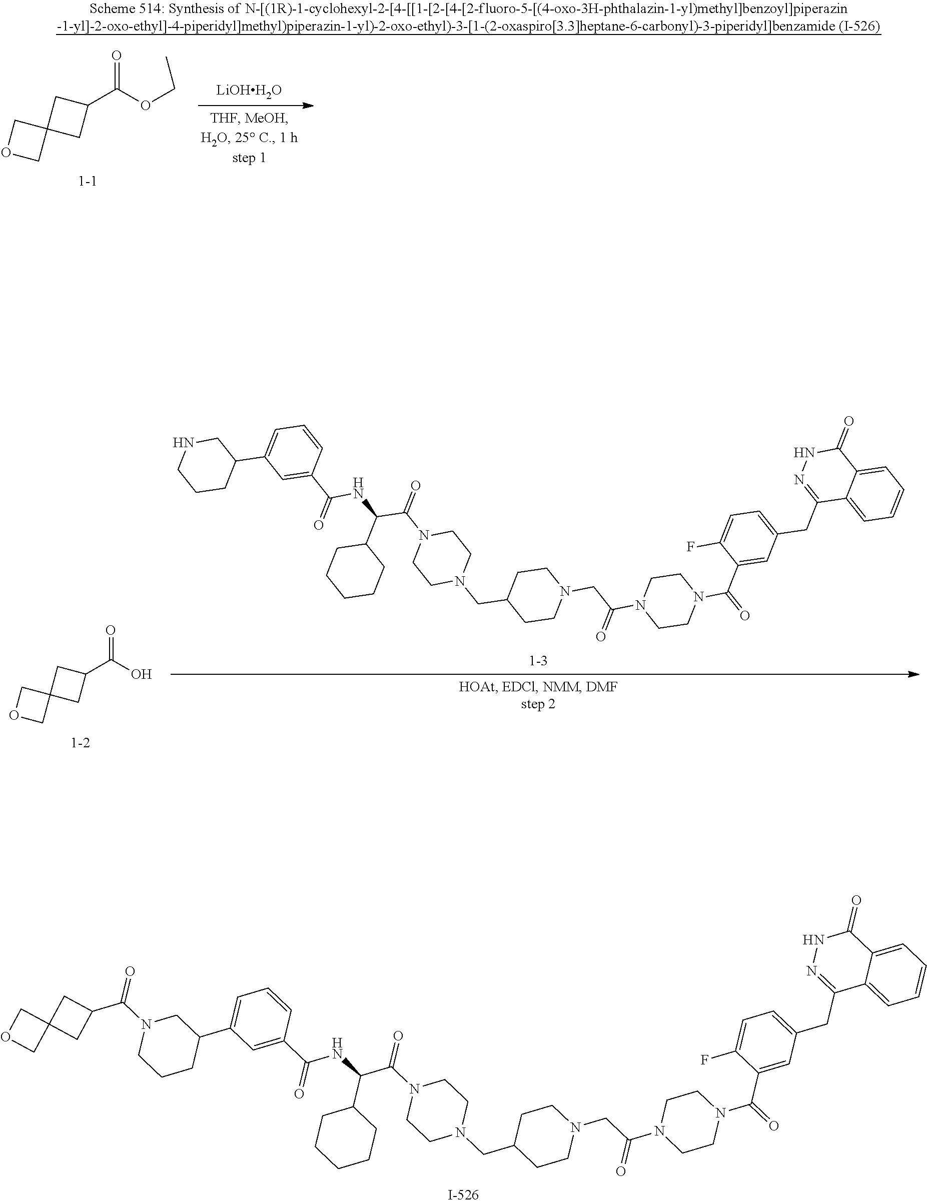 [SMILES: CCOC(=O)C1CC2(COC2)C1 ; O=C(O)C1CC2(COC2)C1 ; O=C(N[C@@H](C(=O)N1CCN(CC2CCN(CC(=O)N3CCN(C(=O)c4cc(Cc5n[nH]c(=O)c6ccccc56)ccc4F)CC3)CC2)CC1)C1CCCCC1)c1cccc(C2CCCNC2)c1 ; O=C(N[C@@H](C(=O)N1CCN(CC2CCN(CC(=O)N3CCN(C(=O)c4cc(Cc5n[nH]c(=O)c6ccccc56)ccc4F)CC3)CC2)CC1)C1CCCCC1)c1cccc(C2CCCN(C(=O)C3CC4(COC4)C3)C2)c1]

### Step 1: Synthesis of Intermediate 1-2

To a solution of Intermediate 1-1 (50 mg, 293.76 μmol, 1 eq) in THF (0.5 mL), MeOH (0.5 mL) and H₂O (0.5 mL) was added LiOH·H₂O (24.65 mg, 587.53 μmol, 2 eq). The mixture was stirred at 25° C. for 1 hr. The mixture was adjusted to pH=3 with aq·HCl (1 N) and extracted with EA. The organic layer was dried with anhydrous Na₂SO₄, filtered and concentrated under vacuum. This residue was used in the next step without further purification. Intermediate 1-2 (20 mg, 140.69 μmol, 47.89% yield) was obtained as a colorless oil.

### Step 2: Synthesis of I-526

To a solution of Intermediate 1-2 (18.62 mg, 130.99 μmol, 1 eq) in DMF (1 mL) was added HOAt (17.83 mg, 130.99 μmol, 18.32 μL, 1 eq), EDCI (50.22 mg, 261.97 μmol, 2 eq), NMM (66.24 mg, 654.93 μmol, 72.00 μL, 5 eq) and Intermediate 1-3 (0.12 g, 130.99 μmol, 1 eq). The mixture was stirred at 25° C. for 1 h. The reaction mixture was quenched by H₂O (2 mL) and extracted with EA (1 mL*3). The combined organic layers were concentrated under reduced pressure to give a residue. The residue was purified by Prep-HPLC (column: Waters Xbridge 150*25 mm*5 um; mobile phase: [water (NH₄HCO₃)-ACN]; gradient: 28%-58% B over 15 min). I-526 (63.81 mg, 61.34 μmol, 46.83% yield) was obtained as a white solid. LCMS (Method G): Rt=0.631 min, [M+H]⁺=1040.8. SFC: Rt=3.647 min, 4.594 min ¹H NMR (400 MHz, METHANOL-d₄) δ=8.40-8.34 (m, 1H), 7.97-7.91 (m, 1H), 7.86-7.84 (m, 2H), 7.77-7.67 (m, 2H), 7.52-7.36 (m, 4H), 7.18-7.14 (m, 1H), 4.94-4.87 (m, 1H), 4.74-4.65 (m, 2H), 4.61-4.50 (m, 3H), 4.38 (s, 2H), 3.85-3.64 (m, 8H), 3.58-3.46 (m, 3H), 3.27-3.05 (m, 4H), 2.95-2.90 (m, 2H), 2.78-2.64 (m, 2H), 2.53-2.35 (m, 8H), 2.24-2.00 (m, 5H), 1.94-1.50 (m, 13H), 1.39-1.03 (m, 8H).

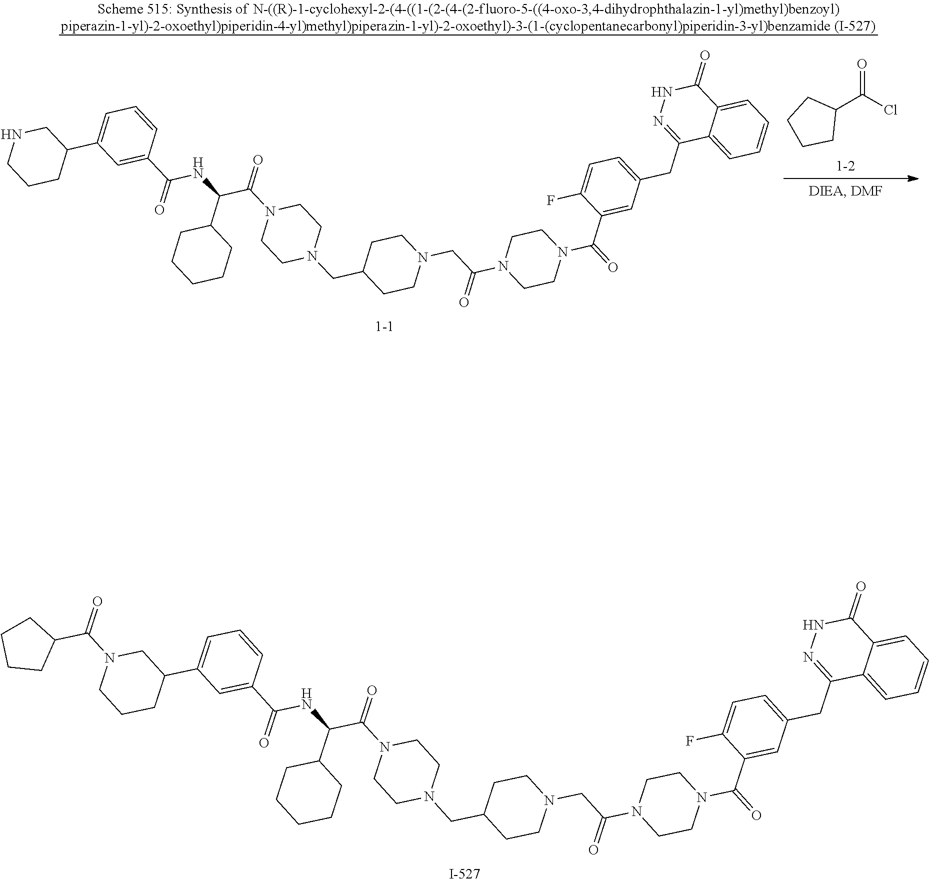

To a solution of Intermediate 1-1 (100 mg, 109.15 μmol, 1 eq) and Intermediate 1-2 (21.71 mg, 163.73 μmol, 19.90 μL, 1.5 eq) in DMF (1 mL) was added DIEA (42.32 mg, 327.46 μmol, 57.04 μL, 3 eq). The mixture was stirred at 25° C. for 0.5 hr. The mixture was directly purified by Prep-HPLC (column: Waters Xbridge 150*25 mm*5 um; mobile phase: [water (NH₄HCO₃)-ACN]; gradient: 32%-62% B over 15 min). The eluent was concentrated to remove MeCN and then lyophilized to afford the product. I-527 (44.45 mg, 43.47 μmol, 39.83% yield, 99% purity) was obtained as a white solid. LCMS (Method G): Rt=0.714 min, M+H=1012.7. SFC: Rt=2.483 min, 3.008 min ¹H NMR (400 MHz, METHANOL-d₄) δ =8.37 (d, J=7.6 Hz, 1H), 7.98-7.93 (m, 1H), 7.91-7.80 (m, 2H), 7.78-7.67 (m, 2H), 7.52-7.35 (m, 4H), 7.20-7.13 (m, 1H), 4.93-4.88 (m, 1H), 4.66-4.57 (m, 1H), 4.39 (s, 2H), 4.19-4.08 (m, 1H), 3.87-3.65 (m, 7H), 3.59-3.49 (m, 3H), 3.21-3.03 (m, 3H), 2.97-2.86 (m, 2H), 2.80-2.63 (m, 2H), 2.54-2.34 (m, 4H), 2.26-2.17 (m, 2H), 2.14-2.02 (m, 3H), 1.97-1.83 (m, 6H), 1.82-1.49 (m, 16H), 1.37-1.00 (m, 8H).

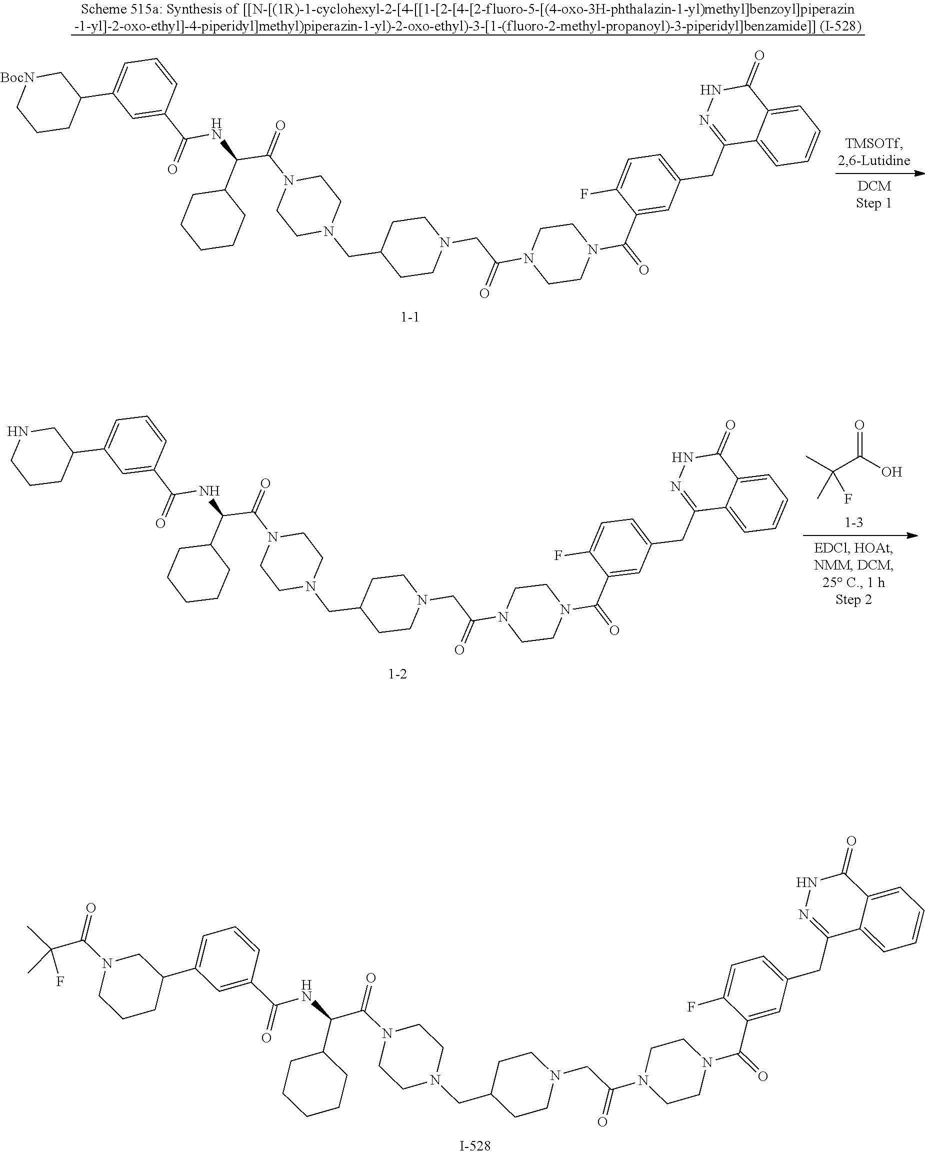

### Step 1: Synthesis of Intermediate 1-2

To a solution of Intermediate 1-1 (2 g, 1.97 mmol, 1 eq) in DCM (20 mL) was added TMSOTf (4.37 g, 19.68 mmol, 3.56 mL, 10 eq) and 2,6-lutidine (316.32 mg, 2.95 mmol, 343.82 μL, 1.5 eq). The mixture was stirred at 0° C. for 0.5 hr. The mixture was concentrated under reduced pressure to give a residue. The crude product was diluted with H₂O (20 mL) at 0° C. and the mixture was stirred for 5 min. Precipitate formed, which was collected by filtration. The collected precipitate was dried under vacuum to afford the crude product. Intermediate 1-2 (1.73 g, 1.89 mmol, 95.95% yield, 100% purity) was obtained as a yellow solid. LCMS (Method D): Rt: 0.283 min, [M+H]⁺=916.4. ¹H NMR (400 MHz, METHANOL-d4) δ=8.36-8.29 (m, 1H), 7.98-7.91 (m, 1H), 7.89-7.77 (m, 3H), 7.69 (d, J=8.0 Hz, 1H), 7.53-7.37 (m, 4H), 7.15-7.14 (m, 1H), 4.81-4.75 (m, 1H), 4.41-4.31 (m, 3H), 4.25 (s, 1H), 3.86-3.65 (m, 8H), 3.60-3.51 (m, 2H), 3.49-3.33 (m, 7H), 3.26-3.02 (m, 8H), 2.29-1.82 (m, 10H), 1.81-1.61 (m, 6H), 1.41-1.02 (m, 6H). ¹⁹F NMR (376 MHz, METHANOL-d4) δ=−78.373.

### Step 2: Synthesis of I-528

To a solution of Intermediate 1-3 (11.58 mg, 109.15 μmol, 1 eq) in DCM (1.5 mL) was added NMM (55.20 mg, 545.77 μmol, 60.00 μL, 5 eq), EDCI (62.78 mg, 327.46 μmol, 3 eq) and HOAt (14.86 mg, 109.15 μmol, 15.27 μL, 1 eq) at 25° C. for 0.5 hr. Then Intermediate 1-2 (100 mg, 109.15 μmol, 1 eq) was added into the mixture and it was stirred at 25° C. for 0.5 hr. The reaction mixture was diluted with water (10 mL) and extracted with DCM (10 mL×3). The combined organic layers were washed with brine (10 mL×3) and dried over sodium sulfate, filtered and concentrated under reduced pressure to give a residue. The crude product was purified by prep-HPLC (column: CD04-Welch Utimate C18 150*25*7 um; mobile phase: [water (FA)-ACN]; gradient: 12%-42% B over 8 min) and the eluent was concentrated under reduced pressure to remove ACN, and then was lyophilized to give I-528 (43.36 mg, 40.81 μmol, 37.38% yield, 98.839% purity, FA salt) as a white solid. LCMS (Method D): Rt=0.358 min, [M+H]⁺=1004.5. ¹H NMR (400 MHz, METHANOL-d4) δ=8.45-8.35 (m, 2H), 7.99-7.93 (m, 1H), 7.91-7.81 (m, 2H), 7.77-7.69 (m, 2H), 7.55-7.41 (m, 3H), 7.39-7.38 (m, 1H), 7.18 (s, 1H), 4.91 (s, 1H), 4.60-4.49 (m, 2H), 4.39 (s, 2H), 3.93-3.69 (m, 7H), 3.62-3.52 (m, 3H), 3.41 (s, 5H), 3.25-3.12 (m, 1H), 2.91-2.64 (m, 4H), 2.60-2.37 (m, 4H), 2.30 (d, J=6.0 Hz, 2H), 2.04 (s, 1H), 2.01-1.85 (m, 6H), 1.79 (d, J=3.2 Hz, 3H), 1.73-1.54 (m, 9H), 1.52-1.40 (m, 2H), 1.35-1.20 (m, 3H), 1.17-1.05 (m, 2H). ¹⁹F NMR (377 MHz, METHANOL-d4) δ=−120.715, −142.673. SFC: Rt: 3.572 min, 3.993 min.

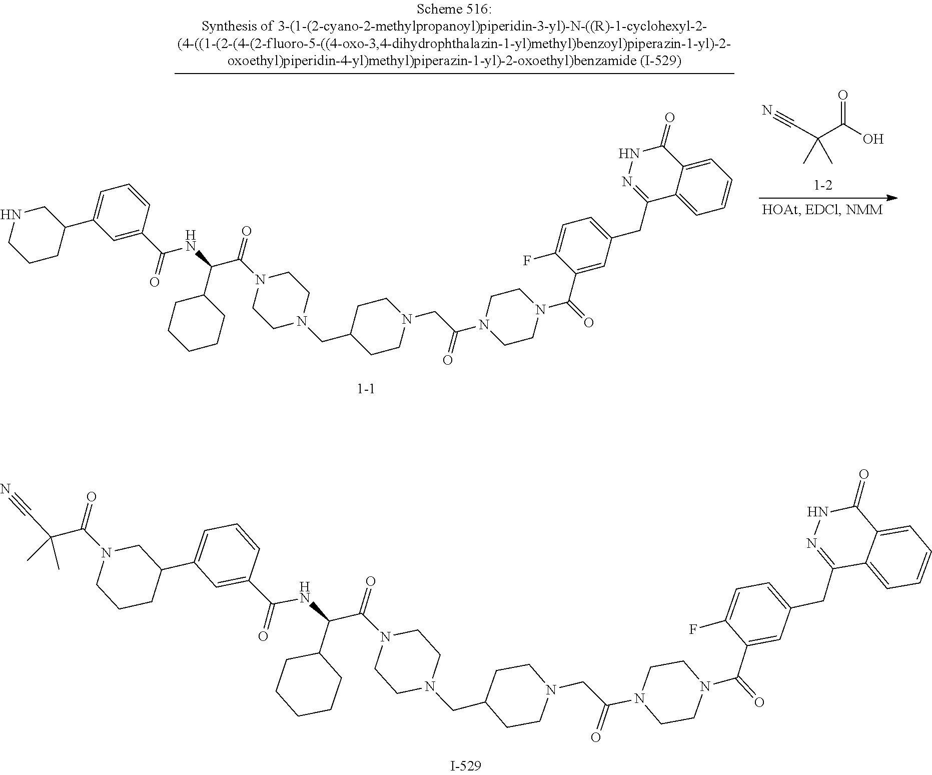

To a solution of Intermediate 1-1 (100 mg, 109.15 μmol, 1 eq) and Intermediate 1-2 (18.52 mg, 163.73 μmol, 1.5 eq) in DMF (1 mL) was added EDCI (104.63 mg, 545.77 μmol, 5 eq), NMM (110.41 mg, 1.09 mmol, 120.01 μL, 10 eq) and HOAt (44.57 mg, 327.46 μmol, 45.81 μL, 3 eq). The mixture was stirred at 25° C. for 0.5 hr. The mixture was directly purified by Prep-HPLC (column: Waters Xbridge 150*25 mm*5 um; mobile phase: [water (NH₄HCO₃)-ACN]; gradient: 32%-62% B over 15 min). The eluent was concentrated to remove MeCN and then lyophilized to afford the product. I-529 (46.64 mg, 45.66 μmol, 41.83% yield, 99% purity) was obtained as a white solid. LCMS (Method G): Rt=0.681 min, M+H=1011.7. SFC: Rt=4.231 min, 4.660 min. ¹H NMR (400 MHz, METHANOL-d₄) δ=8.38 (d, J=7.2 Hz, 1H), 7.99-7.92 (m, 1H), 7.91-7.75 (m, 3H), 7.74-7.68 (m, 1H), 7.57-7.34 (m, 4H), 7.21-7.12 (m, 1H), 4.93-4.88 (m, 1H), 4.81-4.69 (m, 1H), 4.65-4.43 (m, 2H), 4.39 (s, 2H), 3.89-3.63 (m, 7H), 3.60-3.47 (m, 3H), 3.40-3.34 (m, 1H), 3.27-3.12 (m, 3H), 2.97-2.83 (m, 3H), 2.57-2.33 (m, 4H), 2.24-2.19 (m, 2H), 2.15-2.01 (m, 3H), 1.98-1.85 (m, 4H), 1.83-1.66 (m, 7H), 1.65-1.51 (m, 7H), 1.38 (s, 8H).

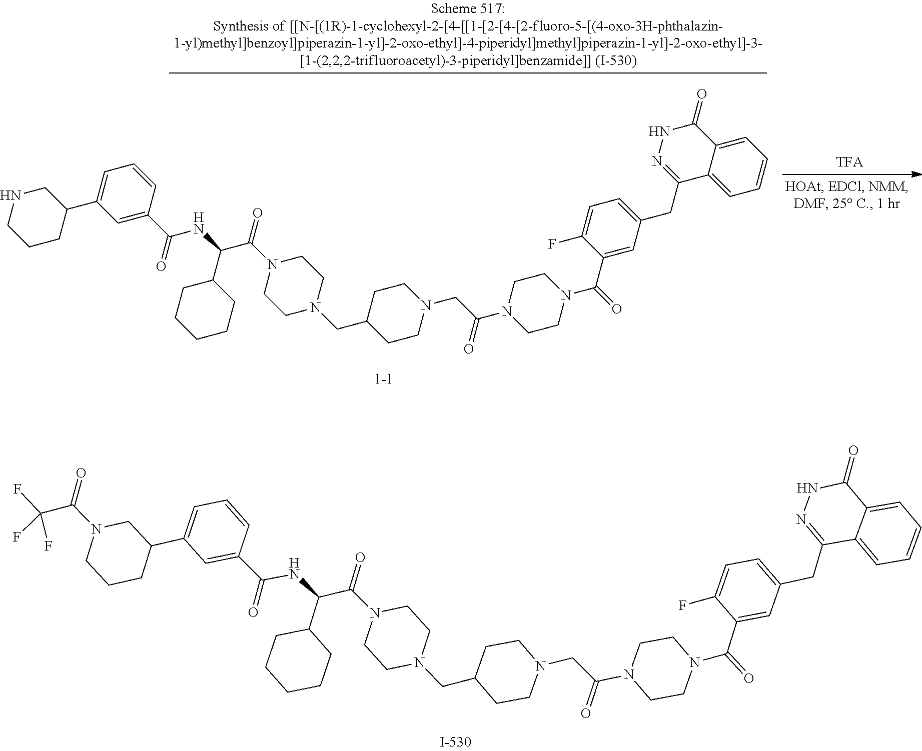 [SMILES: O=C(N[C@@H](C(=O)N1CCN(CC2CCN(CC(=O)N3CCN(C(=O)c4cc(Cc5n[nH]c(=O)c6ccccc56)ccc4F)CC3)CC2)CC1)C1CCCCC1)c1cccc(C2CCCNC2)c1 ; O=C(N[C@@H](C(=O)N1CCN(CC2CCN(CC(=O)N3CCN(C(=O)c4cc(Cc5n[nH]c(=O)c6ccccc56)ccc4F)CC3)CC2)CC1)C1CCCCC1)c1cccc(C2CCCN(C(=O)C(F)(F)F)C2)c1]

To a solution of TFA (14.94 mg, 130.99 μmol, 9.73 μL, 1.2 eq) in DMF (1 mL) was added HOAt (14.86 mg, 109.15 μmol, 15.27 μL, 1 eq), EDCI (62.78 mg, 327.46 μmol, 3 eq), NMM (55.20 mg, 545.77 μmol, 60.00 μL, 5 eq) and intermediate 1-1 (100 mg, 109.15 μmol, 1 eq). The mixture was stirred at 25° C. for 1 hr. The reaction mixture was diluted with H₂O (2 mL) and extracted with EA (2 mL*3). The combined organic layers were washed by brine (2 mL*3), dried over Na₂SO₄, filtered and concentrated under reduced pressure to give a residue. The residue was purified by reversed phase chromatography (FA condition). The eluent was concentrated and lyophilized to give I-530 (55.48 mg, 51.48 μmol, 47.16% yield, 98.182% purity, FA salt) as a white solid. LCMS (Method D): Retention time: 0.363 min, [M+H]⁺=1012.4. SFC: Retention time: 3.645, 3.874 min ¹H NMR (400 MHz, METHANOL-d4) δ=8.52 (s, 1H), 8.40-8.33 (m, 1H), 7.98-7.92 (m, 1H), 7.89-7.80 (m, 2H), 7.79-7.69 (m, 2H), 7.52-7.41 (m, 3H), 7.38 (s, 1H), 7.18-7.13 (m, 1H), 4.91 (d, J=8.8 Hz, 1H), 4.84-4.81 (m, 1H), 4.61-4.47 (m, 1H), 4.38 (s, 2H), 4.12-3.93 (m, 1H), 3.88-3.65 (m, 7H), 3.54 (br dd, J=4.1, 16.0 Hz, 3H), 3.29-3.18 (m, 3H), 3.03-2.82 (m, 4H), 2.53-2.34 (m, 4H), 2.23-2.20 (m, 2H), 2.14-2.02 (m, 3H), 1.97-1.84 (m, 4H), 1.82-1.66 (m, 7H), 1.62-1.51 (m, 1H), 1.46-1.16 (m, 6H), 1.16-1.06 (m, 2H). ¹⁹F NMR (377 MHz, METHANOL-d4) δ=−120.77, −70.405.

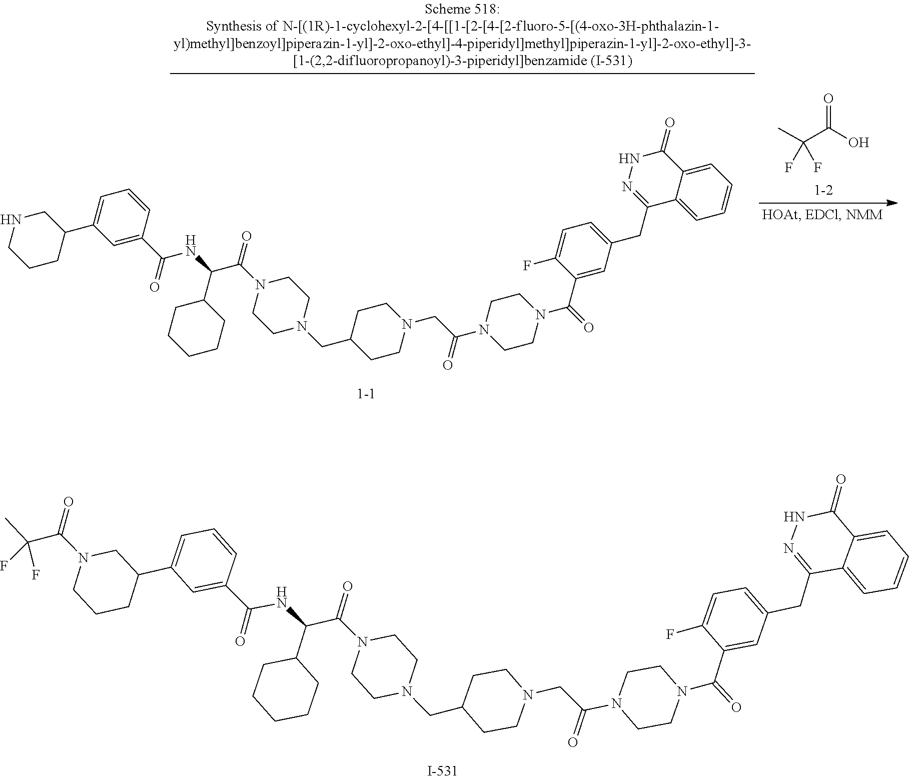

To a solution of Intermediate 1 (100.00 mg, 104.98 μmol, 1 eq) in DMF (1 mL) was added Intermediate 2 (11.55 mg, 104.98 μmol, 1 eq), EDCI (40.25 mg, 209.95 μmol, 2 eq), NMM (53.09 mg, 524.88 μmol, 57.71 μL, 5 eq) and HOAt (14.29 mg, 104.98 μmol, 14.68 μL, 1 eq). The mixture was stirred at 25° C. for 1 hr. The mixture was diluted with water (5 mL) and extracted with EtOAc (10 mL*3). The organic layer was concentrated under vacuum. The residue was purified by Prep-HPLC (column: Waters Xbridge 150*25 mm*5 um; mobile phase: [water (NH₄HCO₃)-ACN]; gradient: 42%-72% B over 9 min) and dried by lyophilization. I-531 (38.25 mg, 37.53 μmol, 97.01% yield, 98.91% purity) was obtained as a white solid. LCMS (Method G): Rt=0.695 min, [M+H]⁺=1008.8. SFC: PRt=3.200 min, 3.568 min. ¹H NMR (400 MHz, METHANOL-d₄) δ=8.40-8.35 (m, 1H), 7.98-7.93 (m, 1H), 7.90-7.82 (m, 2H), 7.79-7.70 (m, 2H), 7.52-7.43 (m, 3H), 7.41-7.35 (m, 1H), 7.21-7.13 (m, 1H), 4.60-4.53 (m, 1H), 4.39 (s, 2H), 4.36-4.27 (m, 1H), 3.89-3.65 (m, 8H), 3.59-3.50 (m, 3H), 3.27-3.19 (m, 3H), 2.96-2.85 (m, 3H), 2.83-2.77 (m, 1H), 2.52-2.36 (m, 4H), 2.23-2.18 (m, 2H), 2.11-2.03 (m, 3H), 1.95-1.89 (m, 3H), 1.87 (br d, J=4.0 Hz, 2H), 1.84-1.73 (m, 7H), 1.73-1.64 (m, 3H), 1.61-1.53 (m, 1H), 1.37-1.19 (m, 6H), 1.16-1.07 (m, 2H). ¹⁹F NMR (377 MHz, METHANOL-d₄) δ=−93.024, −93.328, −93.680, −93.912, −120.808.

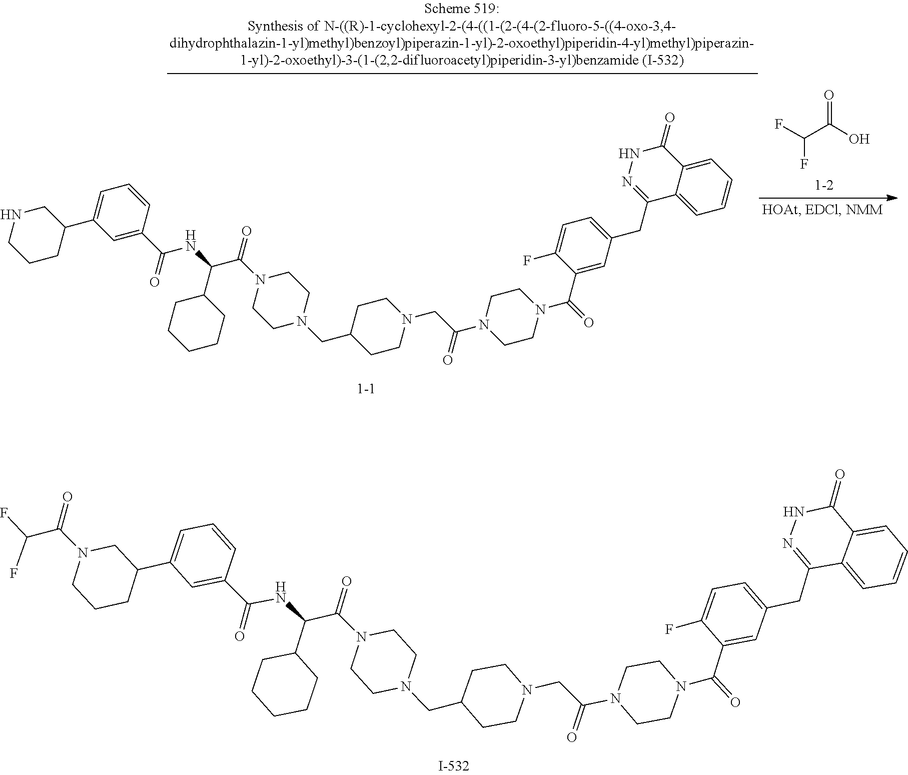 [SMILES: O=C(N[C@@H](C(=O)N1CCN(CC2CCN(CC(=O)N3CCN(C(=O)c4cc(Cc5n[nH]c(=O)c6ccccc56)ccc4F)CC3)CC2)CC1)C1CCCCC1)c1cccc(C2CCCNC2)c1 ; O=C(O)C(F)F ; O=C(N[C@@H](C(=O)N1CCN(CC2CCN(CC(=O)N3CCN(C(=O)c4cc(Cc5n[nH]c(=O)c6ccccc56)ccc4F)CC3)CC2)CC1)C1CCCCC1)c1cccc(C2CCCN(C(=O)C(F)F)C2)c1]

To a solution of Intermediate 1-1 (100 mg, 109.15 μmol, 1 eq) and Intermediate 1-2 (15.72 mg, 163.73 μmol, 10.30 μL, 1.5 eq) in DMF (1 mL) was added EDCI (104.63 mg, 545.77 μmol, 5 eq), NMM (110.41 mg, 1.09 mmol, 120.01 μL, 10 eq) and HOAt (44.57 mg, 327.46 μmol, 45.81 μL, 3 eq). The mixture was stirred at 25° C. for 0.5 hr. The mixture was directly purified by Prep-HPLC (column: Waters Xbridge 150*25 mm*5 um; mobile phase: [water (NH₄HCO₃)-ACN]; gradient: 32%-62% B over 15 min). The eluent was concentrated to remove MeCN and then lyophilized to afford the product. I-532 (50.87 mg, 50.66 μmol, 46.41% yield, 99% purity) was obtained as a white solid. LCMS (Method G): Rt=0.666 min, M+H=994.6. SFC: Rt=3.007 min, 5.554 min. ¹H NMR (400 MHz, METHANOL-d₄) δ=8.37 (d, J=7.6 Hz, 1H), 7.98-7.92 (m, 1H), 7.91-7.81 (m, 2H), 7.78-7.69 (m, 2H), 7.53-7.42 (m, 3H), 7.38 (br s, 1H), 7.19-7.14 (m, 1H), 6.67-6.33 (m, 1H), 4.92-4.88 (m, 1H), 4.83-4.74 (m, 1H), 4.60-4.50 (m, 1H), 4.39 (s, 2H), 4.10-4.00 (m, 1H), 3.87-3.66 (m, 7H), 3.59-3.49 (m, 3H), 3.26-3.12 (m, 3H), 2.95-2.76 (m, 4H), 2.55-2.34 (m, 4H), 2.26-2.17 (m, 2H), 2.14-2.01 (m, 3H), 1.96-1.84 (m, 4H), 1.82-1.64 (m, 7H), 1.61-1.50 (m, 1H), 1.18 (br s, 8H).

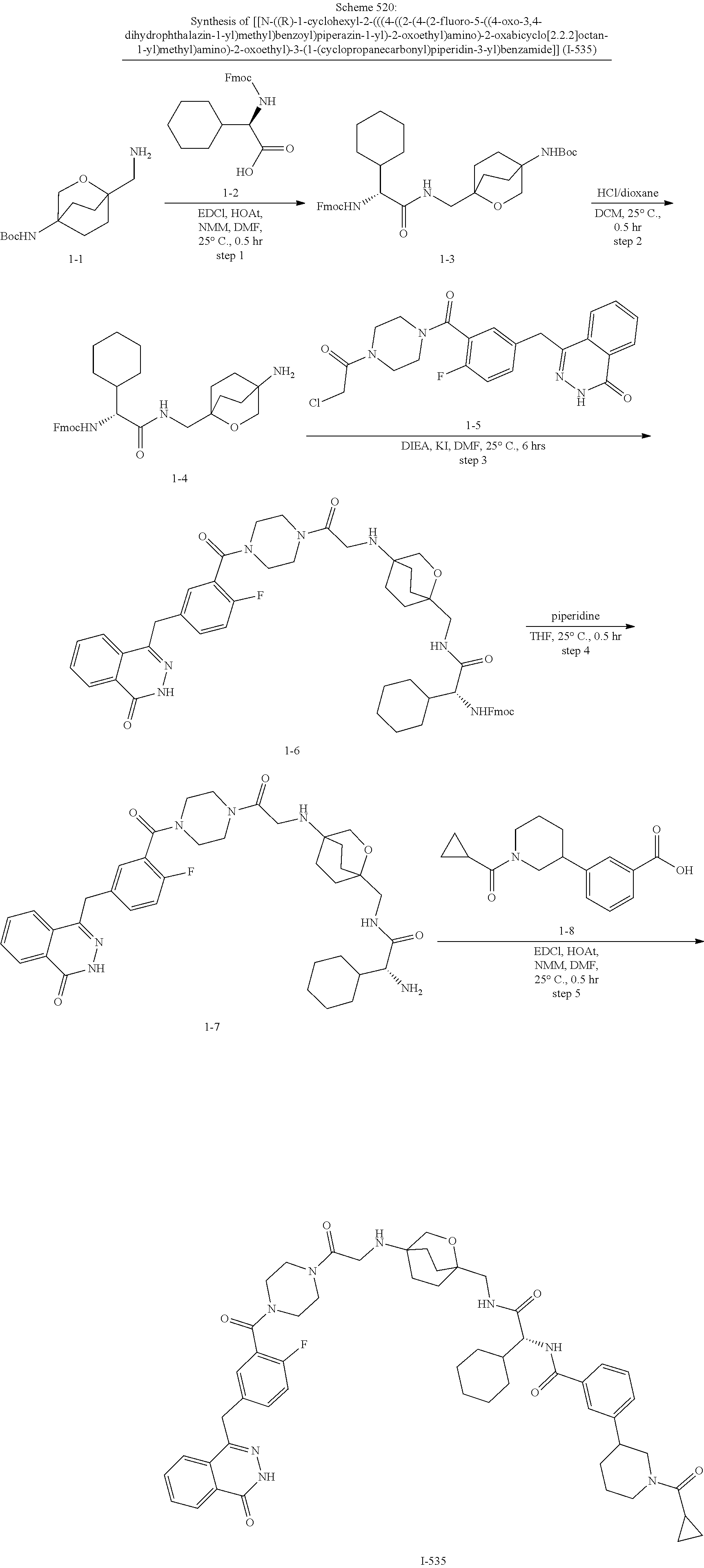

### Step 1: Synthesis of Intermediate 1-3.

To a solution of Intermediate 1-2 (1.11 g, 2.93 mmol, 1.11 mL, 1 eq) in DMF (10 mL) was added HOAt (398.23 mg, 2.93 mmol, 409.28 μL, 1 eq), EDCI (1.68 g, 8.78 mmol, 3 eq) and the mixture was stirred for 0.5 hr. Then Intermediate 1-1 and NMM (1.48 g, 14.63 mmol, 1.61 mL, 5 eq) was added. The mixture was stirred at 25° C. for 1 hr. The reaction mixture was diluted with H₂O (20 mL) and extracted with EA (20 mL*3). The combined organic layers were washed with brine (30 mL), dried over Na₂SO₄, filtered and concentrated under reduced pressure to give a residue. The residue was purified by flash silica gel chromatography (ISCO; 40 g SepaFlash Silica Flash Column, Eluent of 0˜50% Ethyl acetate/Petroleum ether gradient @60 mL/min). Then the eluent was concentrated under reduced pressure to give a residue. Intermediate 1-3 (1 g, 1.60 mmol, 54.52% yield, 98.541% purity) was obtained as a white solid. LCMS (Method D): Retention time: 0.536 min, [M+H]⁺=618.3. SFC: Retention time: 0.685 min. ¹H NMR (400 MHz, METHANOL-d4) δ=7.80 (d, J=7.6 Hz, 2H), 7.70-7.64 (m, 2H), 7.41-7.36 (m, 2H), 7.34-7.26 (m, 2H), 4.44-4.35 (m, 2H), 4.26-4.20 (m, 1H), 3.95-3.82 (m, 3H), 3.19-3.07 (m, 2H), 2.06-1.96 (m, 2H), 1.86-1.73 (m, 6H), 1.70-1.58 (m, 5H), 1.40 (s, 9H), 1.33-1.14 (m, 4H), 1.11-0.94 (m, 2H).

### Step 2: Synthesis of Intermediate 1-4.

To a solution of Intermediate 1-3 (1 g, 1.62 mmol, 1 eq) in DCM (5 mL) was added HCl/dioxane (2 M, 5 mL). The mixture was stirred at 25° C. for 0.5 hr. The reaction mixture was concentrated under reduced pressure to give a residue. The crude product was used in the next step without further purification. Intermediate 1-4 (900 mg, crude, HCl salt) was obtained as a white solid. LCMS (Method D): Retention time: 0.380 min, [M+H]⁺=518.3. ¹H NMR (400 MHz, METHANOL-d4) δ=7.80 (d, J=7.6 Hz, 2H), 7.67 (d, J=7.6 Hz, 2H), 7.42-7.36 (m, 2H), 7.34-7.27 (m, 2H), 4.41-4.19 (m, 3H), 3.92-3.86 (m, 1H), 3.81-3.74 (m, 2H), 3.23-3.09 (m, 2H), 1.89 (d, J=6.0 Hz, 5H), 1.79-1.59 (m, 8H), 1.31-0.96 (m, 6H).

### Step 3: Synthesis of Intermediate 1-6.

To a solution of Intermediate 1-4 (500 mg, 902.33 μmol, 1 eq, HCl salt) and Intermediate 1-5 (399.62 mg, 902.33 μmol, 1 eq) in DMF (5 mL) was added DIEA (116.62 mg, 902.33 μmol, 157.17 μL, 1 eq) and KI (149.79 mg, 902.33 μmol, 1 eq). The mixture was stirred at 25° C. for 6 hr. The reaction mixture was diluted with H₂O (10 mL) and extracted with EA (10 mL*3). The combined organic layers were washed with brine (10 mL*3), dried over Na₂SO₄, filtered and concentrated under reduced pressure to give a residue. The crude product was purified by reversed phase chromatography (0.1% FA condition). Then the eluent was concentrated and lyophilized to give the desired product. Intermediate 1-6 (200 mg, 195.48 μmol, 21.66% yield, 94.818% purity, FA salt) was obtained as a white solid. LCMS (Method D): Retention time: 0.426 min, [M+H]⁺=924.3. SFC: Retention time: 2.149 min. ¹H NMR (400 MHz, METHANOL-d4) δ=8.44 (s, 1H), 8.37 (d, J=8.0 Hz, 1H), 7.94 (d, J=2.0 Hz, 1H), 7.90-7.73 (m, 4H), 7.66 (d, J=1.2 Hz, 2H), 7.55-7.47 (m, 1H), 7.45-7.25 (m, 5H), 7.21-7.12 (m, 1H), 4.89 (s, 1H), 4.42-4.32 (m, 4H), 4.25-4.19 (m, 1H), 3.91 (d, J=7.6 Hz, 1H), 3.80-3.71 (m, 2H), 3.66 (d, J=10.0 Hz, 3H), 3.51 (s, 3H), 3.43-3.38 (m, 1H), 3.37-3.32 (m, 2H), 3.15 (s, 2H), 1.90-1.59 (m, 14H), 1.33-0.97 (m, 5H).

### Step 4: Synthesis of Intermediate 1-7.

To a solution of Intermediate 1-6 (150 mg, 154.62 μmol, 1 eq, FA salt) in THF (1.6 mL) was added piperidine (0.2 mL). The mixture was stirred at 25° C. for 0.5 hr. The reaction mixture was concentrated under reduced pressure to give a residue. The crude product was purified by reversed phase chromatography (neutral condition) and the eluent was concentrated and lyophilized to give the desired product. Intermediate 1-7 (80 mg, 112.89 μmol, 73.01% yield, 99.041% purity) was obtained as a yellow solid. LCMS (Method D): Retention time: 0.274 min, [M+H]⁺=702.4. SFC: Retention time: 0.969 min. ¹H NMR (400 MHz, METHANOL-d4) δ=8.37 (d, J=7.8 Hz, 1H), 7.98-7.93 (m, 1H), 7.91-7.80 (m, 2H), 7.49 (s, 1H), 7.41-7.35 (m, 1H), 7.21-7.13 (m, 1H), 4.39 (s, 2H), 3.81-3.72 (m, 2H), 3.68 (d, J=6.8 Hz, 3H), 3.59-3.50 (m, 2H), 3.48 (s, 1H), 3.40 (s, 2H), 3.36-3.33 (m, 1H), 3.23-3.17 (m, 1H), 3.15-3.09 (m, 2H), 2.96 (s, 1H), 1.92-1.83 (m, 2H), 1.79-1.61 (m, 13H), 1.31-1.14 (m, 4H).

### Step 5: Synthesis of I-535

To a solution of Intermediate 1-7 (51.35 mg, 73.17 μmol, 1 eq) in DMF (0.5 mL) was added HOAt (9.96 mg, 73.17 μmol, 10.24 μL, 1 eq), EDCI (42.08 mg, 219.52 μmol, 3 eq) and NMM (37.01 mg, 365.86 μmol, 40.22 μL, 5 eq). The mixture was stirred at 25° C. for 0.5 hr. The reaction mixture was diluted with H₂O (10 mL) and extracted with EA (10 mL*3). The combined organic layers were washed with brine (10 mL*3), dried over Na₂SO₄, filtered and concentrated under reduced pressure to give a residue. The crude product was purified by reversed phase chromatography (0.1% FA condition). Then the eluent was concentrated and lyophilized to give the desired product. I-535 (26.19 mg, 25.38 μmol, 34.68% yield, 97.196% purity, FA salt) was obtained as a white solid. LCMS (Method D): Retention time: 0.395 min, [M+H]⁺=957.7. SFC: Retention time: 2.466, 3.026 min. ¹H NMR (400 MHz, METHANOL-d4) δ=8.45 (s, 2H), 8.37 (d, J=7.2 Hz, 1H), 7.95 (d, J=7.2 Hz, 1H), 7.90-7.67 (m, 4H), 7.54-7.33 (m, 4H), 7.21-7.12 (m, 1H), 4.61-4.52 (m, 1H), 4.39 (s, 4H), 3.81-3.65 (m, 5H), 3.61-3.48 (m, 4H), 3.42-3.34 (m, 2H), 3.25-3.08 (m, 3H), 2.88-2.68 (m, 2H), 2.11-1.97 (m, 2H), 1.93-1.62 (m, 17H), 1.43-0.96 (m, 6H), 0.92-0.72 (m, 4H). ¹⁹F NMR (377 MHz, METHANOL-d4) δ=−120.78.

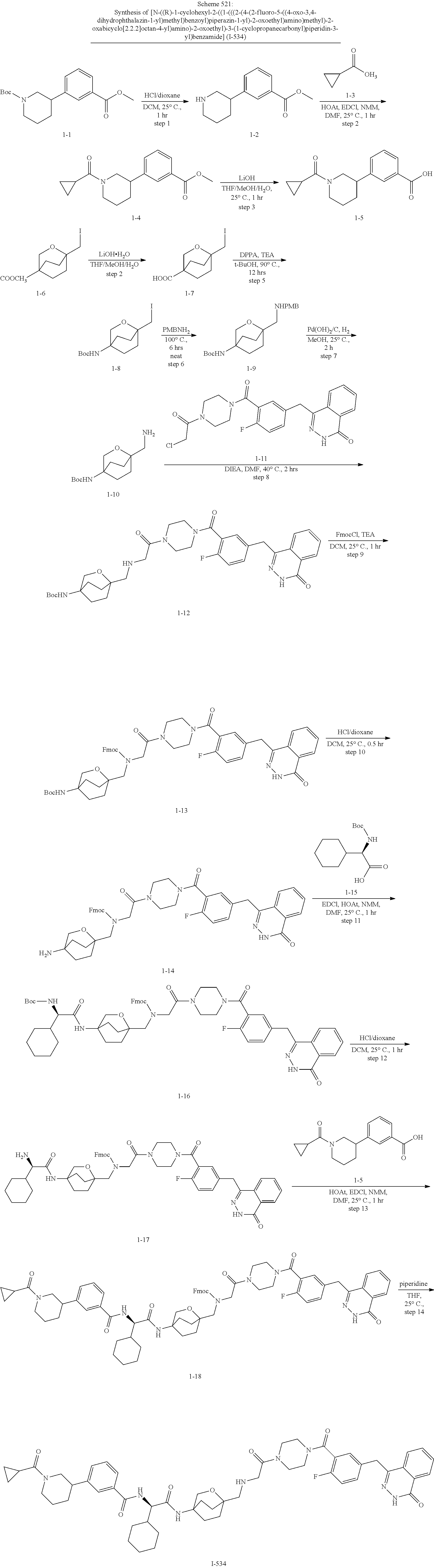

### Step 1: Synthesis of Intermediate 1-2.

To a solution of Intermediate 1-1 (10 g, 31.31 mmol, 1 eq) in DCM (50 mL) was added HCl/dioxane (2 M, 50 mL). The mixture was stirred at 25° C. for 0.5 hr. The reaction mixture was concentrated under reduced pressure to give Intermediate 1-2 (8 g, 31.28 mmol, 99.91% yield, HCl salt) as a white solid. LCMS (Method D): Retention time: 0.221 min, [M+H]⁺=220.1.

### Step 2: Synthesis of Intermediate 1-4.

To a solution of Intermediate 1-3 (2.6 g, 30.20 mmol, 2.39 mL, 1 eq) and Intermediate 1-2 (7.72 g, 30.20 mmol, 1 eq, HCl salt) in DMF (25 mL) was added HOAt (4.11 g, 30.20 mmol, 4.22 mL, 1 eq), EDCI (17.37 g, 90.60 mmol, 3 eq) and NMM (15.27 g, 151.01 mmol, 16.60 mL, 5 eq). The mixture was stirred at 25° C. for 1 hr. The reaction mixture was diluted with H₂O (50 mL) and extracted with EA (50 mL*2). The combined organic layers were washed with brine (50 mL*2), dried over Na₂SO₄, filtered and concentrated under reduced pressure to give a residue. The residue was purified by flash silica gel chromatography (ISCO; 120 g SepaFlash Silica Flash Column, Eluent of 0˜35% Ethyl acetate/Petroleum ether gradient @60 mL/min), the eluent was concentrated under reduced pressure to give Intermediate 1-4 (7 g, 24.20 mmol, 80.12% yield, 99.326% purity) as a yellow oil. LCMS (Method D): Retention time: 0.364 min, [M+H]⁺=288.2. ¹H NMR (400 MHz, CHLOROFORM-d) δ=7.97-7.91 (m, 2H), 7.50-7.37 (m, 2H), 4.85-4.24 (m, 2H), 3.94 (s, 3H), 3.04-2.75 (m, 2H), 2.11 (d, J=12.8 Hz, 1H), 1.93-1.73 (m, 3H), 1.70-1.56 (m, 2H), 1.06-0.97 (m, 2H), 0.82-0.73 (m, 2H).

### Step 3: Synthesis of Intermediate 1-5.

To a solution of Intermediate 1-4 (7 g, 24.36 mmol, 1 eq) in THF (20 mL), MeOH (20 mL) and H₂O (20 mL) was added LiOH·H₂O (3.07 g, 73.08 mmol, 3 eq). The mixture was stirred at 25° C. for 1 hr. The mixture was adjusted to pH=5 with 1M HCl and extracted with EA (50 mL*3), the organic layers were dried over Na₂SO₄, filtered and concentrated to give crude product. The crude product was used in the next step without further purification. Intermediate 1-5 (5 g, 18.29 mmol, 75.09% yield) was obtained as a yellow oil. LCMS (Method D): Retention time: 0.351 min, [M+H]⁺=274.2. ¹H NMR (400 MHz, METHANOL-d4) δ=8.01-7.90 (m, 2H), 7.61-7.42 (m, 2H), 4.64-4.55 (m, 1H), 4.47-4.35 (m, 1H), 3.30-3.22 (m, 1H), 2.90-2.71 (m, 2H), 2.13-1.84 (m, 5H), 0.95-0.78 (m, 4H).

### Step 4: Synthesis of Intermediate 1-7.

To a solution of Intermediate 1-6 (8 g, 25.80 mmol, 1 eq) in MeOH (30 mL), THF (30 mL) and H₂O (30 mL) was added LiOH·H₂O (1.62 g, 38.69 mmol, 1.5 eq). The mixture was stirred at 25° C. for 1 hr. The mixture was adjusted to pH=3 with 1M HCl and extracted with EA (50 mL*3), the organic layers were dried over Na₂SO₄, filtered and concentrated to give a crude product. Intermediate 1-7 (6.1 g, 20.60 mmol, 79.86% yield) was obtained as a white solid. ¹H NMR (400 MHz, CHLOROFORM-d) δ=4.01 (s, 2H), 3.17 (s, 2H), 2.05-1.92 (m, 6H), 1.83-1.72 (m, 2H).

### Step 5: Synthesis of Intermediate 1-8.

To a solution of Intermediate 1-7 (6 g, 20.26 mmol, 1 eq) in t-BuOH (80 mL) was dropwise added TEA (2.15 g, 21.28 mmol, 2.96 mL, 1.05 eq) and DPPA (5.58 g, 20.26 mmol, 4.37 mL, 1 eq). The mixture was stirred at 90° C. for 12 hr. Then the mixture was cooled to 25° C. and concentrated to give a residue. The residue was dissolved in EtOAc (80 mL) and then a solution of NaHCO₃ (86.36 g, 1.03 μmol, 40.00 mL, 50.73 eq) was added, and the mixture was stirred at 25° C. for 2 hrs. The mixture was poured into water (200 mL) and extracted with EA (200 mL*3), the combined organic layers were dried over Na₂SO₄, filtered and concentrated to give a residue. The residue was purified by flash silica gel chromatography (ISCO; 120 g SepaFlash Silica Flash Column, Eluent of 0˜10% Ethyl acetate/Petroleum ethergradient @60 mL/min), and the eluent was concentrated under reduced pressure to give Intermediate 1-8 (3.5 g, 9.53 mmol, 47.04% yield) as a pale yellow solid. LCMS (Method D): Retention time: 0.434 min, [M-Boc+H]⁺=268.1. ¹H NMR (400 MHz, CHLOROFORM-d) δ=3.96 (s, 2H), 3.15 (s, 2H), 2.14-1.96 (m, 4H), 1.91-1.76 (m, 4H), 1.42 (s, 9H).

### Step 6: Synthesis of Intermediate 1-9.

To a solution of Intermediate 1-8 (3.5 g, 9.53 mmol, 1 eq) was added (4-methoxyphenyl)methanamine (2.61 g, 19.06 mmol, 2.47 mL, 2 eq). The mixture was stirred at 100° C. for 6 hr. The reaction mixture was concentrated under reduced pressure to give a residue. The residue was purified by reversed phase chromatography (0.1% FA condition), the eluent was concentrated and lyophilized to give Intermediate 1-9 (1.65 g, 3.91 mmol, 40.97% yield, FA salt) as a yellow oil. LCMS (Method D): Retention time: 0.307 min, [M+H]⁺=377.4. ¹H NMR (400 MHz, METHANOL-d4) δ=8.07 (s, 1H), 7.40 (d, J=8.8 Hz, 2H), 6.99 (d, J=8.4 Hz, 2H), 4.14 (s, 2H), 3.96 (s, 2H), 3.82 (s, 3H), 2.87 (s, 2H), 2.13-2.03 (m, 2H), 1.97-1.81 (m, 4H), 1.76-1.65 (m, 2H), 1.40 (s, 9H).

### Step 7: Synthesis of Intermediate 1-10.

To a mixture of Pd(OH)₂/C (1 g, 20% purity) in MeOH (15 mL) was added Intermediate 1-9 (1.6 g, 3.79 mmol, 1 eq, FA salt). The mixture was degassed and purged with H₂ 3 times, and then the mixture was stirred at 25° C. for 2 hr under H₂ (15 psi) atmosphere. The reaction mixture was filtered and washed with MeOH (30 mL*2). The filtrate was concentrated under reduced pressure to give Intermediate 1-10 (1.2 g, crude) as a pale yellow oil. LCMS (Method D): Retention time: 0.227 min, [M+H]⁺=257.2. ¹H NMR (400 MHz, METHANOL-d4) δ=3.97 (s, 2H), 2.86 (s, 2H), 2.16-2.03 (m, 2H), 1.97-1.86 (m, 4H), 1.79-1.69 (m, 2H), 1.41 (s, 9H).

### Step 8: Synthesis of Intermediate 1-12.

To a solution of Intermediate 1-10 (350 mg, 1.37 mmol, 1 eq) and Intermediate 1-11 (604.68 mg, 1.37 mmol, 1 eq) in DMF (4 mL) was added DIEA (529.39 mg, 4.10 mmol, 713.47 μL, 3 eq). The mixture was stirred at 40° C. for 2 hr. The reaction mixture was diluted with H₂O (10 mL) and extracted with EA (10 mL*3). The combined organic layers were washed with brine (10 mL*3), dried over Na₂SO₄, filtered and concentrated under reduced pressure to give a residue. The residue was purified by flash silica gel chromatography (ISCO; 40 g SepaFlash Silica Flash Column, Eluent of 0˜5% Methanol/Ethyl acetate gradient @60 mL/min), the eluent was concentrated under reduced pressure to give Intermediate 1-12 (600 mg, 905.32 μmol, 66.31% yield) as a yellow solid. LCMS (Method D): Retention time: 0.329 min, [M+H]⁺=663.3. ¹H NMR (400 MHz, METHANOL-d4) δ=8.39-8.34 (m, 1H), 7.97-7.93 (m, 1H), 7.90-7.79 (m, 2H), 7.53-7.45 (m, 1H), 7.42-7.32 (m, 1H), 7.21-7.12 (m, 1H), 4.39 (s, 2H), 3.95 (d, J=14.8 Hz, 2H), 3.81-3.67 (m, 5H), 3.62-3.50 (m, 3H), 2.88-2.83 (m, 1H), 2.67 (d, J=10.4 Hz, 2H), 2.14-2.05 (m, 2H), 2.02-1.95 (m, 2H), 1.92-1.82 (m, 3H), 1.77-1.68 (m, 2H), 1.41 (s, 9H).

### Step 9: Synthesis of Intermediate 1-13.

To a solution of Intermediate 1-12 (500 mg, 754.43 μmol, 1 eq) in DCM (5 mL) was added TEA (229.02 mg, 2.26 mmol, 315.02 μL, 3 eq) and 9H-fluoren-9-ylmethyl carbonochloridate (214.69 mg, 829.88 μmol, 1.1 eq). The mixture was stirred at 25° C. for 0.5 hr. The reaction mixture was concentrated under reduced pressure to give a residue. The residue was purified by flash silica gel chromatography (ISCO; 20 g SepaFlash Silica Flash Column, Eluent of 0˜5% Methanol/Ethyl acetate gradient @60 mL/min), and the eluent was concentrated under reduced pressure to give Intermediate 1-13 (300 mg, 323.75 μmol, 42.91% yield, 95.506% purity) as a pale yellow solid. LCMS (Method D): Retention time: 0.502 min, [M+H]⁺=885.5. ¹H NMR (400 MHz, METHANOL-d4) δ=8.36 (d, J=7.6 Hz, 1H), 7.93 (d, J=7.2 Hz, 1H), 7.89-7.69 (m, 4H), 7.63-7.52 (m, 2H), 7.46 (s, 1H), 7.43-7.09 (m, 6H), 4.72-4.59 (m, 2H), 4.44-4.34 (m, 2H), 4.25-4.03 (m, 3H), 3.90 (s, 1H), 3.82-3.58 (m, 6H), 3.44 (s, 2H), 3.21-3.11 (m, 2H), 2.66 (s, 1H), 2.05-1.90 (m, 2H), 1.84-1.65 (m, 4H), 1.56 (d, J=4.0 Hz, 2H), 1.40 (s, 9H).

### Step 10: Synthesis of Intermediate 1-14.

To a solution of Intermediate 1-13 (300 mg, 338.99 μmol, 1 eq) in DCM (2 mL) was added HCl/dioxane (2 M, 2 mL). The mixture was stirred at 25° C. for 0.5 hr. The reaction mixture was concentrated under reduced pressure to give Intermediate 1-14 (300 mg, crude, HCl salt) as a white solid. LCMS (Method D): Retention time: 0.380 min, [M+H]⁺=785.2.

### Step 11: Synthesis of Intermediate 1-16.

To a solution of Intermediate 1-15 (90 mg, 349.75 μmol, 1 eq) and Intermediate 1-14 (287.26 mg, 349.75 μmol, 1 eq, HCl salt) in DMF (2 mL) was added HOAt (47.61 mg, 349.75 μmol, 48.93 μL, 1 eq), EDCI (201.14 mg, 1.05 mmol, 3 eq) and NMM (176.88 mg, 1.75 mmol, 192.26 μL, 5 eq). The mixture was stirred at 25° C. for 1 hr. The reaction mixture was diluted with H₂O (5 mL) and extracted with EA (10 mL*3). The combined organic layers were washed with brine (10 mL*3), dried over Na₂SO₄, filtered and concentrated under reduced pressure to give a residue. The residue was purified by flash silica gel chromatography (ISCO; 20 g SepaFlash Silica Flash Column, Eluent of 0˜80% Ethyl acetate/Petroleum ethergradient @60 mL/min), and the eluent was concentrated under reduced pressure to give Intermediate 1-16 (260 mg, 220.98 μmol, 63.18% yield, 87.046% purity) as a white solid. LCMS (Method D): Retention time: 0.486 min, [M+H]⁺=1024.3. ¹H NMR (400 MHz, METHANOL-d4) δ=8.36 (d, J=7.6 Hz, 1H), 7.96-7.79 (m, 4H), 7.63-7.45 (m, 3H), 7.42-7.27 (m, 5H), 7.22-7.09 (m, 1H), 6.40 (d, J=7.6 Hz, 1H), 4.71 (d, J=2.8 Hz, 1H), 4.62 (d, J=4.4 Hz, 1H), 4.42-4.36 (m, 2H), 4.23-4.10 (m, 2H), 3.90-3.64 (m, 5H), 3.63-3.39 (m, 3H), 3.35 (s, 2H), 3.26-3.10 (m, 3H), 2.67 (s, 1H), 2.14-2.05 (m, 1H), 1.94-1.81 (m, 2H), 1.77-1.54 (m, 9H), 1.47-1.40 (m, 9H), 1.15-0.76 (m, 7H).

### Step 12: Synthesis of Intermediate 1-16.

To a solution of Intermediate 1-15 (200 mg, 195.28 μmol, 1 eq) in DCM (2 mL) was added HCl/dioxane (2 M). The mixture was stirred at 25° C. for 0.5 hr. The reaction mixture was concentrated under reduced pressure to give Intermediate 1-16 (200 mg, crude, HCl salt) as a yellow solid. LCMS (Method D): Retention time: 0.410 min, [M+H]⁺=924.4. SFC: Retention time: 1.936 min.

### Step 13: Synthesis of Intermediate 1-18.

To a solution of Intermediate 1-17 (90 mg, 93.70 μmol, 1 eq, HCl salt) and Intermediate 1-5 (25.61 mg, 93.70 μmol, 1 eq) in DMF (1 mL) was added HOAt (12.75 mg, 93.70 μmol, 13.11 μL, 1 eq), EDCI (53.89 mg, 281.10 μmol, 3 eq) and NMM (47.39 mg, 468.49 μmol, 51.51 μL, 5 eq). The mixture was stirred at 25° C. for 0.5 hr. The reaction mixture was diluted with H₂O (5 mL) and extracted with EA (5 mL*3). The combined organic layers were washed with brine (5 mL*3), dried over Na₂SO₄, filtered and concentrated under reduced pressure to give a residue. The residue was purified by flash silica gel chromatography (ISCO; 12 g SepaFlash Silica Flash Column, Eluent of 0˜100% Ethyl acetate/Petroleum ether gradient @40 mL/min), the eluent was concentrated under reduced pressure to give Intermediate 1-18 (60 mg, 38.31 μmol, 40.88% yield, 75.298% purity) as a yellow solid. LCMS (Method D): Retention time: 0.535 min, [M+H]⁺=1179.2.

### Step 14: Synthesis of I-534

To a solution of Intermediate 1-18 (40 mg, 33.92 μmol, 1 eq) in THF (0.8 mL) was added piperidine (0.1 mL). The mixture was stirred at 25° C. for 0.5 hr. The reaction mixture was concentrated under reduced pressure to give a residue. The residue was purified by reversed phase chromatography (neutral condition), the eluent was concentrated and lyophilized to give the desired product. I-534 (18.84 mg, 19.68 μmol, 58.04% yield, 100% purity) was obtained as a white solid. LCMS (Method H): Retention time: 0.630 min, [M+H]⁺=957.6. SFC: Retention time: 2.839, 3.324 min. ¹H NMR (400 MHz, METHANOL-d4) δ=8.37 (d, J=7.6 Hz, 1H), 7.97-7.92 (m, 1H), 7.90-7.81 (m, 2H), 7.80-7.67 (m, 2H), 7.53-7.34 (m, 4H), 7.19-7.10 (m, 1H), 4.91 (s, 1H), 4.61-4.51 (m, 1H), 4.38 (s, 3H), 4.30 (d, J=7.2 Hz, 1H), 4.05-3.95 (m, 2H), 3.81-3.66 (m, 3H), 3.63-3.39 (m, 5H), 3.24-3.02 (m, 1H), 2.88-2.61 (m, 2H), 2.50 (d, J=6.0 Hz, 2H), 2.21-1.90 (m, 8H), 1.88-1.49 (m, 12H), 1.34-1.02 (m, 5H), 0.93-0.72 (m, 4H). ¹⁹F NMR (376 MHz, METHANOL-d4) δ=−120.78.

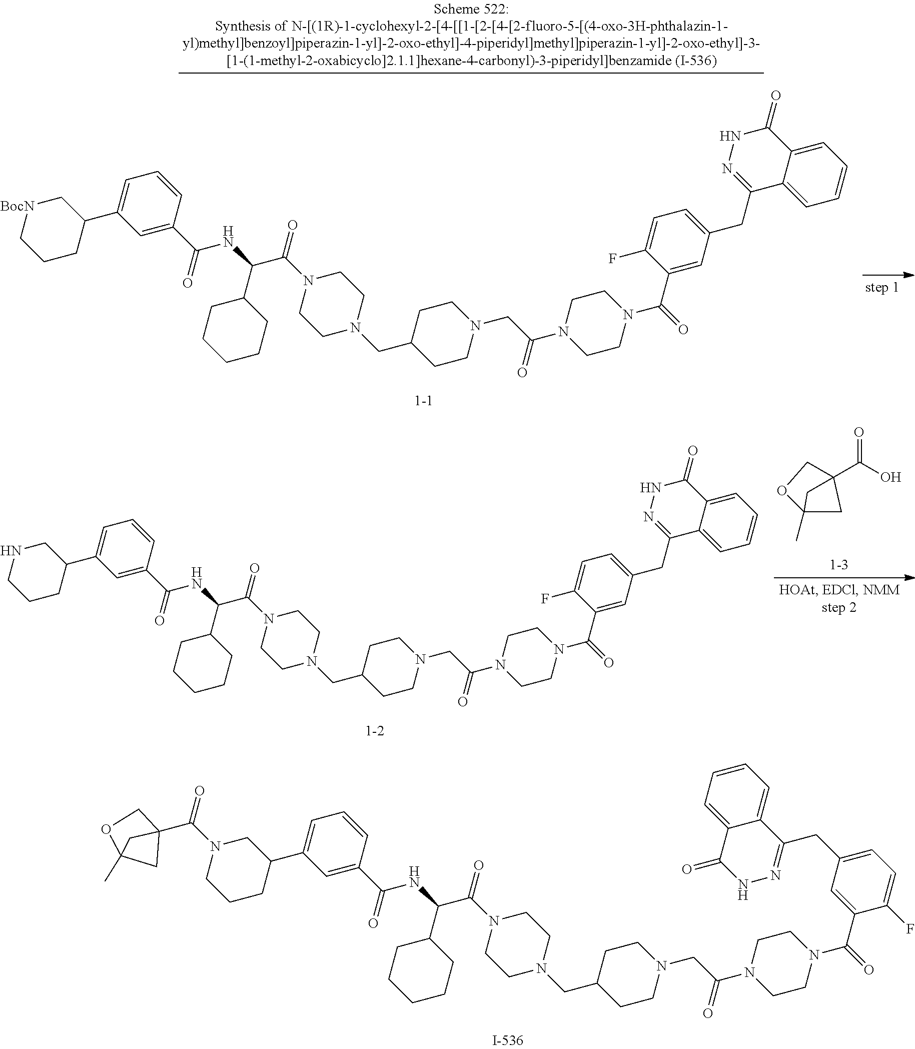

### Step 1: Synthesis of Intermediate 1-2

To a solution of Intermediate 1-1 (4.5 g, 4.43 mmol, 1 eq) in DCM (45 mL) was added 2,6-dimethylpyridine (142.34 mg, 1.33 mmol, 154.72 μL, 0.3 eq) and trimethylsilyl trifluoromethanesulfonate (1.48 g, 6.64 mmol, 1.20 mL, 1.5 eq). The mixture was stirred at 0° C. for 0.5 hr. The mixture was quenched by NH₃·H₂O to pH=8 and concentrated under vacuum to give a residue. The residue was purified by reversed-phase HPLC (0.1% NH₃·H₂O). Intermediate 1-2 (3.9 g, 4.18 mmol, 94.41% yield, 98.208% purity) was obtained as a yellow solid. LCMS (Method E): Rt=0.383 min, [M+H]⁺=916.7. SFC: Rt=2.324 min, 2.941 min. ¹H NMR (400 MHz, DMSO-d₆) δ=12.64 (s, 1H), 8.53 (br d, J=8.1 Hz, 1H), 8.31 (br d, J=7.6 Hz, 1H), 8.03-7.98 (m, 1H), 7.93 (t, J=7.1 Hz, 1H), 7.90-7.81 (m, 3H), 7.53-7.46 (m, 3H), 7.42 (br t, J=6.6 Hz, 1H), 7.28 (t, J=9.0 Hz, 1H), 4.80 (br t, J=8.5 Hz, 1H), 4.38 (s, 2H), 3.66-3.21 (m, 21H), 3.16-2.92 (m, 5H), 2.40-2.31 (m, 3H), 2.19-2.14 (m, 2H), 1.96 (br d, J=7.9 Hz, 3H), 1.82-1.61 (m, 9H), 1.24-1.00 (m, 7H).

### Step 2: Synthesis of I-536

To a mixture of Intermediate 1-2 (0.1 g, 109.15 μmol, 1 eq), Intermediate 1-3 (15.52 mg, 109.15 μmol, 1 eq) and HOAt (14.86 mg, 109.15 μmol, 15.27 μL, 1 eq) in DMF (1 mL) was added EDCI (41.85 mg, 218.31 μmol, 2 eq) and NMM (55.20 mg, 545.77 μmol, 60.01 μL, 5 eq). The mixture was stirred at 25° C. for 2 hrs. The mixture was diluted with water (10 mL) and extracted with EtOAc (10 mL*3). The organic layer was concentrated under vacuum. The residue was purified by Prep-HPLC (column: Waters Xbridge 150*25 mm*5 um; mobile phase: [water (NH₄HCO₃)-ACN]; gradient: 36%-66% B over 9 min) and dried by lyophilized. I-536 (69.49 mg, 65.71 μmol, 60.19% yield, 98.361% purity) as an off-white solid was obtained. LCMS (Method): Rt=1.839 min, [M+H]⁺=1040.9 SFC: Rt=2.976 min, 3.295 min ¹H NMR (400 MHz, METHANOL-d₄) δ=8.37 (br d, J=7.5 Hz, 1H), 7.99-7.91 (m, 1H), 7.91-7.79 (m, 2H), 7.78-7.67 (m, 2H), 7.52-7.41 (m, 3H), 7.38 (br s, 1H), 7.16-7.15 (m, 1H), 4.90 (br d, J=9.6 Hz, 1H), 4.63-4.51 (m, 1H), 4.38 (s, 2H), 3.91 (br d, J=5.2 Hz, 1H), 3.85 (br d, J=5.6 Hz, 2H), 3.80 (br d, J=4.4 Hz, 2H), 3.73 (br s, 4H), 3.67 (br d, J=2.4 Hz, 1H), 3.59-3.48 (m, 3H), 3.36-3.33 (m, 1H), 3.28 (br s, 2H), 3.24-3.12 (m, 2H), 2.98-2.85 (m, 2H), 2.82-2.67 (m, 2H), 2.53-2.33 (m, 4H), 2.21-2.20 (m, 2H), 2.15-1.99 (m, 5H), 1.93-1.83 (m, 5H), 1.82-1.73 (m, 5H), 1.69 (br d, J=10.8 Hz, 2H), 1.63-1.51 (m, 2H), 1.45-1.37 (m, 3H), 1.33-1.18 (m, 5H), 1.16-1.04 (m, 2H). ¹⁹F NMR (377 MHz, METHANOL-d₄) δ=−120.720.

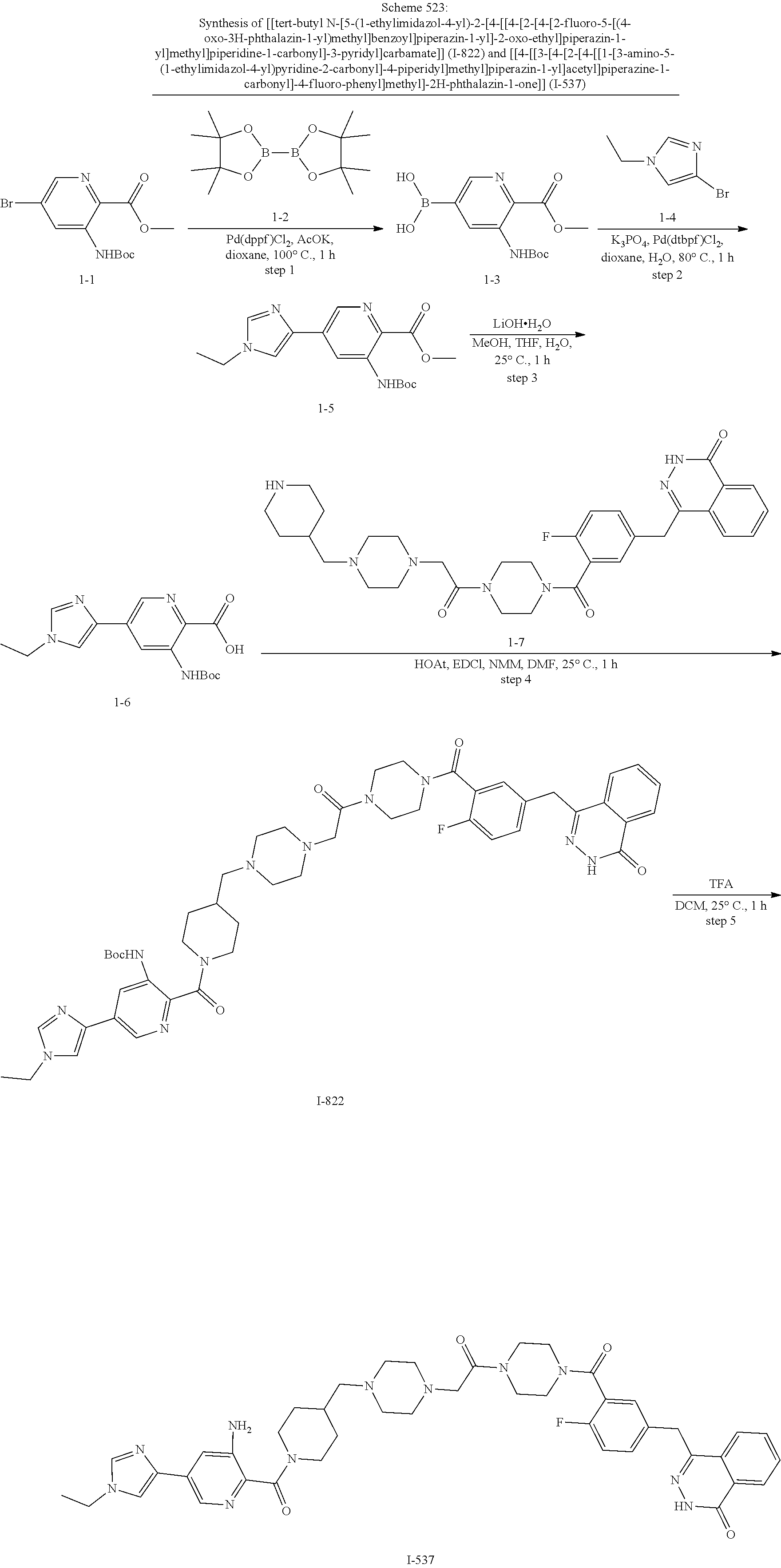

### Step 1: Synthesis of Intermediate 1-3

To a mixture of intermediate 1-1 (3 g, 9.06 mmol, 1 eq) in dioxane (30 mL) was added intermediate 1-2 (6.90 g, 27.18 mmol, 3 eq), Pd(dppf)Cl₂ (662.85 mg, 905.90 μmol, 0.1 eq) and AcOK (2.67 g, 27.18 mmol, 3 eq) at 25° C., the mixture was degassed and purged with N₂ 3 times, and then the mixture was stirred at 100° C. for 1 hr. The reaction mixture was concentrated under reduced pressure to give a residue. The crude product was purified by reversed-phase chromatography (0.1% FA condition) and lyophilized to give a residue. Intermediate 1-3 (1.68 g, 5.45 mmol, 60.13% yield, 96% purity) was obtained as a light yellow solid. LCMS (Method D): Rt=0.393 min, [M+H]⁺=296.8. ¹H NMR (400 MHz, METHANOL-d4) δ=9.04 (s, 1H), 8.45 (s, 1H), 3.99 (s, 3H), 1.55 (s, 9H).

### Step 2: Synthesis of Intermediate 1-5

A mixture of intermediate 1-3 (0.5 g, 1.69 mmol, 1.1 eq), intermediate 1-4 (268.70 mg, 1.54 mmol, 1 eq), K₃PO₄ (977.61 mg, 4.61 mmol, 3 eq) and Pd(dtbpf)Cl₂ (100.06 mg, 153.52 μmol, 0.1 eq) in dioxane (5 mL) and H₂O (1 mL) was degassed and purged with N₂ 3 times. Then the mixture was stirred at 80° C. for 1 hr under N₂ atmosphere. The reaction mixture was concentrated under reduced pressure to give a residue. The residue was purified by flash silica gel chromatography (ISCO; 40 g SepaFlash Silica Flash Column, Eluent of 0˜60% Ethyl acetate/Methanol gradient @60 mL/min) and concentrated under reduced pressure to give a residue. Intermediate 1-5 (1.1 g, 2.73 mmol, 59.30% yield, 86% purity) as a brown solid. LCMS (Method D): Rt=0.307 min, [M+H]⁺=347.1.

### Step 3: Synthesis of Intermediate 1-6

To a solution of intermediate 1-5 (1 g, 2.89 mmol, 1 eq) in THF (4 mL), MeOH (4 mL) and H₂O (2 mL) was added LiOH H₂O (605.74 mg, 14.44 mmol, 5 eq). The mixture was stirred at 25° C. for 1 hr. The reaction mixture was poured into H₂O (10 mL). The precipitate was filtered and washed with DCM (10 mL*2). The resulting solid was dried under reduced pressure to give the crude product, which was used in the next step without further purification. Intermediate 1-6 (950 mg, 2.86 mmol, 99.01% yield, 100% purity) was obtained as a brown solid. LCMS (Method D): Rt=0.274 min, [M+H]⁺=333.1. ¹H NMR (400 MHz, METHANOL-d4) δ=9.03 (s, 1H), 8.55 (s, 1H), 7.74 (d, J=18.8 Hz, 2H), 4.13 (s, 2H), 1.89 (s, 3H), 1.54 (s, 9H).

### Step 4: Synthesis of I-822

To a solution of intermediate 1-6 (30 mg, 90.27 μmol, 1 eq) and intermediate 1-7 (58.55 mg, 99.29 μmol, 1.1 eq) in DMF (0.5 mL) was added EDCI (51.91 mg, 270.80 μmol, 3 eq), HOAT (12.29 mg, 90.27 μmol, 12.63 μL, 1 eq) and NMM (45.65 mg, 451.33 μmol, 49.62 μL, 5 eq). The mixture was stirred at 25° C. for 1 hr. The reaction mixture was concentrated under reduced pressure to give a residue. The residue was purified by flash silica gel chromatography (ISCO; 4 g SepaFlash Silica Flash Column, Eluent of 0˜60% Ethyl acetate/Methanol gradient @30 mL/min) and concentrated under reduced pressure to give a residue. The crude product was further purified by reversed phase chromatography (Neutral condition) and lyophilized to give I-822 (16.09 mg, 17.80 μmol, 19.72% yield, 100% purity) as a white solid. LCMS (Method D): Rt=0.305 min, [M+H]⁺=904.7. ¹H NMR (400 MHz, METHANOL-d4) δ=8.65 (s, 1H), 8.47 (s, 1H), 8.37 (d, J=7.2 Hz, 1H), 7.94 (s, 1H), 7.91-7.81 (m, 2H), 7.77 (d, J=14.0 Hz, 2H), 7.48 (d, J=2.0 Hz, 1H), 7.37 (s, 1H), 7.16 (t, J=8.4 Hz, 1H), 4.69-4.56 (m, 1H), 4.38 (s, 2H), 4.18-4.09 (m, 2H), 3.83-3.64 (m, 5H), 3.53 (d, J=19.2 Hz, 2H), 3.38 (s, 1H), 3.27 (s, 2H), 3.20 (s, 1H), 3.17-3.07 (m, 1H), 2.93-2.82 (m, 1H), 2.72-2.34 (m, 8H), 2.29-2.19 (m, 2H), 1.96-1.82 (m, 2H), 1.79-1.67 (m, 1H), 1.57-1.47 (m, 12H), 1.38-1.23 (m, 2H). ¹⁹F NMR (377 MHz, METHAN OL-d4) δ=−120.809.

### Step 5: Synthesis of I-537

To a solution of I-822 (50 mg, 55.31 μmol, 1 eq) in DCM (0.5 mL) was added TFA (383.75 mg, 3.37 mmol, 0.25 mL, 60.85 eq). The mixture was stirred at 25° C. for 1 hr. The reaction mixture was concentrated under reduced pressure to give a residue. The crude product was purified by reversed phase chromatography (0.1% TFA condition) and lyophilized to give I-537 (31.59 mg, 34.18 μmol, 61.81% yield, 99.334% purity, TFA salt) as a yellow solid. LCMS (Method D): Rt=0.253 min, [M+H]⁺=804.2. ¹H NMR (400 MHz, METHANOL-d4) δ=9.00 (s, 1H), 8.37 (d, J=6.8 Hz, 1H), 8.18 (s, 1H), 8.08 (s, 1H), 7.99-7.92 (m, 1H), 7.91-7.80 (m, 2H), 7.52 (d, J=2.8 Hz, 1H), 7.47 (s, 1H), 7.41-7.31 (m, 1H), 7.17 (t, J=8.8 Hz, 1H), 4.76-4.64 (m, 1H), 4.39 (s, 2H), 4.35-4.29 (m, 2H), 3.85-3.72 (m, 4H), 3.68 (s, 2H), 3.61 (s, 1H), 3.53 (s, 1H), 3.47-3.42 (m, 1H), 3.39 (s, 2H), 3.28-3.13 (m, 5H), 3.06 (d, J=2.4 Hz, 5H), 2.89 (d, J=2.4 Hz, 2H), 2.18-2.07 (m, 1H), 1.99-1.74 (m, 2H), 1.60 (t, J=7.2 Hz, 3H), 1.41-1.29 (m, 2H). ¹⁹F NMR (377 MHz, METHAN OL-d4) δ=−77.242, −120.792.

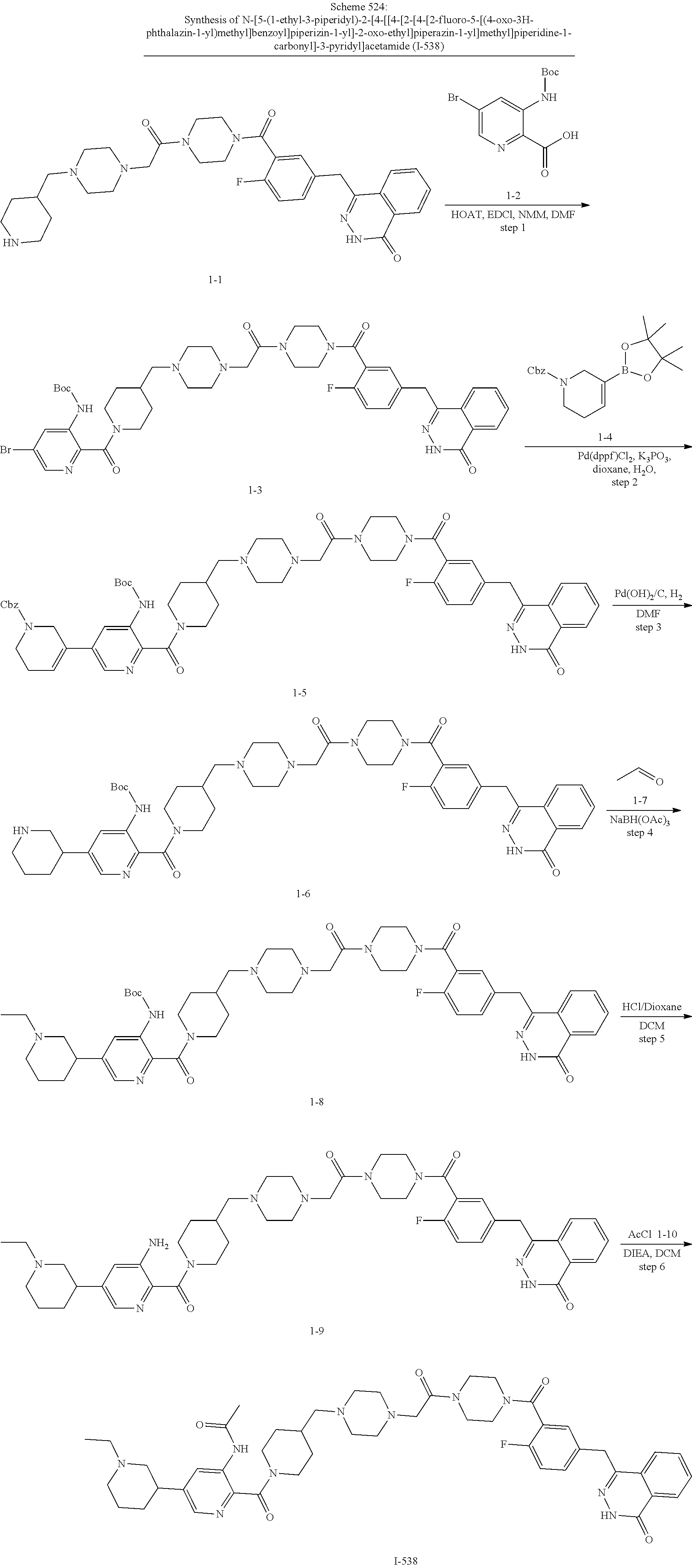

### Step 1: Synthesis of Intermediate 1-3

To a solution of intermediate 1-1 (1 g, 1.70 mmol, 1 eq) and intermediate 1-2 (591.57 mg, 1.87 mmol, 1.1 eq) in DMF (10 mL) was added EDCI (975.24 mg, 5.09 mmol, 3 eq), HOAt (230.81 mg, 1.70 mmol, 237.22 μL, 1 eq) and NMM (857.61 mg, 8.48 mmol, 932.19 μL, 5 eq), then the mixture was stirred at 40° C. for 1 hour. The reaction mixture was diluted with water (80 mL) and extracted with DCM (30 mL*2). The combined organic layers were concentrated under reduced pressure to give a residue. The residue was purified by flash silica gel chromatography (ISCO; 20 g SepaFlash Silica Flash Column, Eluent of 0˜15% EA: Methanol@60 mL/min). The eluent was concentrated under reduced pressure to give the product. Intermediate 1-3 (700 mg, 708.80 μmol, 41.80% yield, 90% purity) was obtained as a brown solid. LCMS (Method D): Retention time: 0.364 min, [M+H]⁺=888.3.

### Step 2: Synthesis of Intermediate 1-5

A mixture of intermediate 1-3 (700 mg, 787.56 μmol, 1 eq) and intermediate 1-4 (675.77 mg, 1.97 mmol, 2.5 eq) in dioxane (7 mL) and H₂O (1.4 mL) was added K₃PO₄ (501.52 mg, 2.36 mmol, 3 eq) and Pd(dtbpf)Cl₂ (51.33 mg, 78.76 μmol, 0.1 eq). Then the mixture was stirred at 80° C. for 1 hour under N₂. The reaction mixture was diluted with water (80 mL) and extracted with EA (30 mL*2). The combined organic layers were concentrated under reduced pressure to give a residue. The residue was purified by flash silica gel chromatography (ISCO; 40 g SepaFlash Silica Flash Column, Eluent of 0˜11% Ethyl acetate: Methanol@60 mL/min). The eluent was concentrated under reduced pressure to give the product. Intermediate 1-5 (730 mg, 665.90 μmol, 84.55% yield, 93.516% purity) was obtained as a red solid. LCMS (Method D): Retention time: 0.421 min, [M+H]⁺=1025.4.

### Step 3: Synthesis of Intermediate 1-6

To a solution of Pd(OH)₂/C (41.10 mg, 20% purity) in DMF (4 mL) was added intermediate 1-5 (400 mg, 390.18 μmol, 1 eq). The reaction mixture was degassed and purged with H₂ 3 times, and then stirred at 40° C. for 2 hour under H₂ (15 psi) atmosphere. The reaction mixture was filtered and concentrated under reduced pressure to give a residue. The crude product was purified by reversed-phase chromatography (0.1% FA condition). The eluent was concentrated under reduced pressure to remove ACN and then lyophilized to give the product. Intermediate 1-6 (80 mg, 89.58 μmol, 22.96% yield, 100% purity) was obtained as a brown solid. LCMS (Method D): Retention time: 0.286 min, [M+H]⁺=893.6.

### Step 4: Synthesis of Intermediate 1-8

To a solution of intermediate 1-6 (70 mg, 78.38 gmol, 1 eq) and intermediate 1-7 (5.92 mg, 94.06 μmol, 7.54 μL, 1.2 eq) in DCM (3 mL) was added AcOH (9.41 mg, 156.76 μmol, 8.97 μL, 2 eq) and NaBH(OAc)₃ (83.06 mg, 391.91 μmol, 5 eq). Then the mixture was stirred at 20° C. for 0.5 h. To the reaction mixture was added sat. NaHCO₃ aq. (10 mL) to adjust the pH to 7-8, then extracted with EA (10 mL*3). The combined organic layers were concentrated under reduced pressure to give a residue. Intermediate 1-8 (55 mg, 59.71 μmol, 76.18% yield) was obtained as a white solid. LCMS (Method F): Retention time: 0.322 min, [M+H]⁺=921.5.

### Step 5: Synthesis of Intermediate 1-9

To a solution of intermediate 1-8 (45 mg, 48.85 μmol, 1 eq) in DCM (1 mL) was added HCl/dioxane (2 M, 122.14 μL, 5 eq). The mixture was stirred at 20° C. for 1 hour. The reaction mixture was concentrated under reduced pressure to give a residue. Intermediate 1-9 (40 mg, 46.65 μmol, 95.49% yield, HCl salt) was obtained as a white solid. LCMS (Method D): Retention time: 0.255 min, [M+H]⁺=821.4.

### Step 6: Synthesis of I-538

To a solution of intermediate 1-9 (40 mg, 46.65 μmol, 1 eq, HCl salt) in DCM (1 mL) was added TEA (14.16 mg, 139.95 μmol, 19.48 μL, 3 eq) and intermediate 1-10 (21.84 mg, 250.70 μmol, 22.06 L, 5 eq) at 0° C., then the mixture was stirred at 0° C. for 1 hour. The reaction mixture was diluted with water (20 mL) and extracted with EA (20 mL*2). The combined organic layers were dried over anhydrous Na₂SO₄, filtered and concentrated under reduced pressure to give a residue. The crude product was purified by prep-HPLC (column: CD04-Welch Utimate C18 150*25*7 um; mobile phase: [water (FA)-ACN]; gradient: 1%-28% B over 8 min). The eluent was concentrated under reduced pressure to remove ACN and then lyophilized to give the product. I-538 (5.66 mg, 6.13 μmol, 13.14% yield, 98.433% purity, FA salt) was obtained as a white solid. LCMS (Method D): Retention time: 0.261 min, [M+H]⁺=863.6. SFC: Retention time: 5.588 min, 6.141 min. ¹H NMR (400 MHz, METHANOL-d4) δ=8.42 (s, 1H), 8.37 (d, J=8.4 Hz, 2H), 8.03 (s, 1H), 7.95 (d, J=6.8 Hz, 1H), 7.91-7.79 (m, 2H), 7.50 (s, 1H), 7.36 (s, 1H), 7.20-7.17 (m, 1H), 4.61 (d, J=12.4 Hz, 1H), 4.39 (s, 2H), 3.83-3.72 (m, 2H), 3.71-3.54 (m, 5H), 3.51 (s, 3H), 3.43-3.32 (m, 3H), 3.23-3.00 (m, 5H), 2.97-2.68 (m, 10H), 2.60 (s, 2H), 2.25-1.90 (m, 8H), 1.85-1.67 (m, 2H), 1.41-1.25 (m, 5H). ¹⁹F NMR (400 MHz, METHANOL-d₄) δ=−120.672, −120.777.

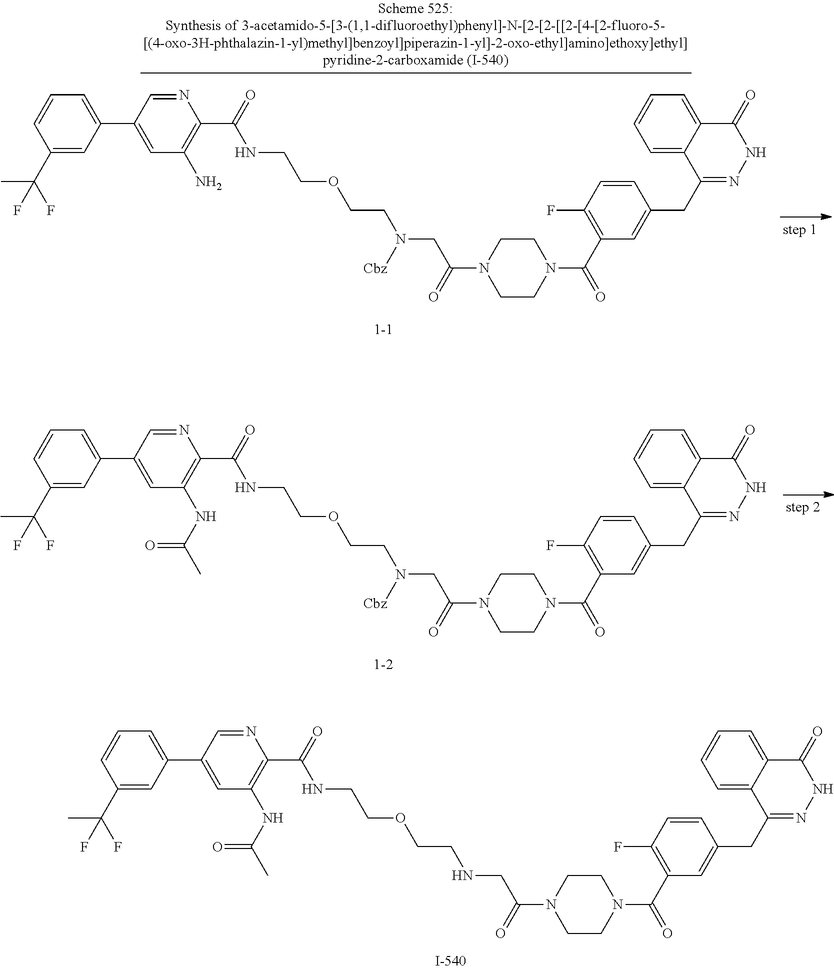 [SMILES: CC(F)(F)c1cccc(-c2cnc(C(=O)NCCOCCN(CC(=O)N3CCN(C(=O)c4cc(Cc5n[nH]c(=O)c6ccccc56)ccc4F)CC3)C(=O)OCc3ccccc3)c(N)c2)c1 ; CC(=O)Nc1cc(-c2cccc(C(C)(F)F)c2)cnc1C(=O)NCCOCCN(CC(=O)N1CCN(C(=O)c2cc(Cc3n[nH]c(=O)c4ccccc34)ccc2F)CC1)C(=O)OCc1ccccc1 ; CC(=O)Nc1cc(-c2cccc(C(C)(F)F)c2)cnc1C(=O)NCCOCCNCC(=O)N1CCN(C(=O)c2cc(Cc3n[nH]c(=O)c4ccccc34)ccc2F)CC1]

### Step 1: Synthesis of Intermediate 1-2

To a solution of Intermediate 1-1 (100 mg, 98.14 μmol, 1 eq, TFA salt) in DCM (1 mL) was added DIEA (38.05 mg, 294.42 μmol, 51.28 μL, 3 eq) and acetyl chloride (8.47 mg, 107.95 μmol, 7.68 μL, 1.1 eq). The mixture was stirred at 0° C. for 1 hr. The reaction mixture was concentrated under reduced pressure to obtain the crude product. The crude product was purified by reversed phase chromatography (0.1% TFA condition). The eluent was concentrated to obtain Intermediate 1-2 (80 mg, 75.40 μmol, 76.83% yield, TFA salt) as a brown oil. LCMS (Method D): Rt=0.495 min, (M+H)=947.1.

### Step 2: Synthesis of I-540

A mixture of Intermediate 1-2 (60 mg, 56.55 μmol, 1 eq, TFA salt), PdCl₂ (5.01 mg, 28.28 μmol, 0.5 eq), Et₃SiH (78.91 mg, 678.61 μmol, 108.39 μL, 12 eq) and TEA (17.17 mg, 169.65 μmol, 23.61 μL, 3 eq) in DCM (0.6 mL) was degassed and purged with N₂ 3 times, and then the mixture was stirred at 25° C. for 1 hr under N₂ atmosphere. The reaction mixture was concentrated under reduced pressure to obtain the crude product. The crude product was diluted with MeOH (5 mL), filtered and concentrated under reduced pressure to give the crude product. The crude product was purified by reversed phase chromatography (0.1% TFA condition), and the eluent was concentrated and lyophilized to give I-540 (21.48 mg, 23.18 μmol, 40.98% yield, 100.00% purity, TFA salt) as a yellow solid. LCMS (Method D): Rt=0.385 min, (M+H)=813.2. ¹H NMR (400 MHz, METHANOL-d₄) δ=9.29 (s, 1H), 8.64-8.58 (m, 1H), 8.36 (d, J=7.6 Hz, 1H), 7.96-7.91 (m, 1H), 7.90-7.85 (m, 1H), 7.82 (s, 2H), 7.81-7.76 (m, 1H), 7.67-7.58 (m, 2H), 7.51-7.48 (m, 1H), 7.32 (t, J=6.8 Hz, 1H), 7.18-7.11 (m, 1H), 4.37 (s, 2H), 4.19-4.07 (m, 2H), 3.87-3.81 (m, 2H), 3.78-3.71 (m, 4H), 3.68 (d, J=2.4 Hz, 3H), 3.53-3.42 (m, 3H), 3.30-3.21 (m, 4H), 2.25 (d, J=3.2 Hz, 3H), 2.04-1.89 (m, 3H). ¹⁹FNMR (377 MHz, METHANOL-d₄), −77.122, −88.807, −120.657.

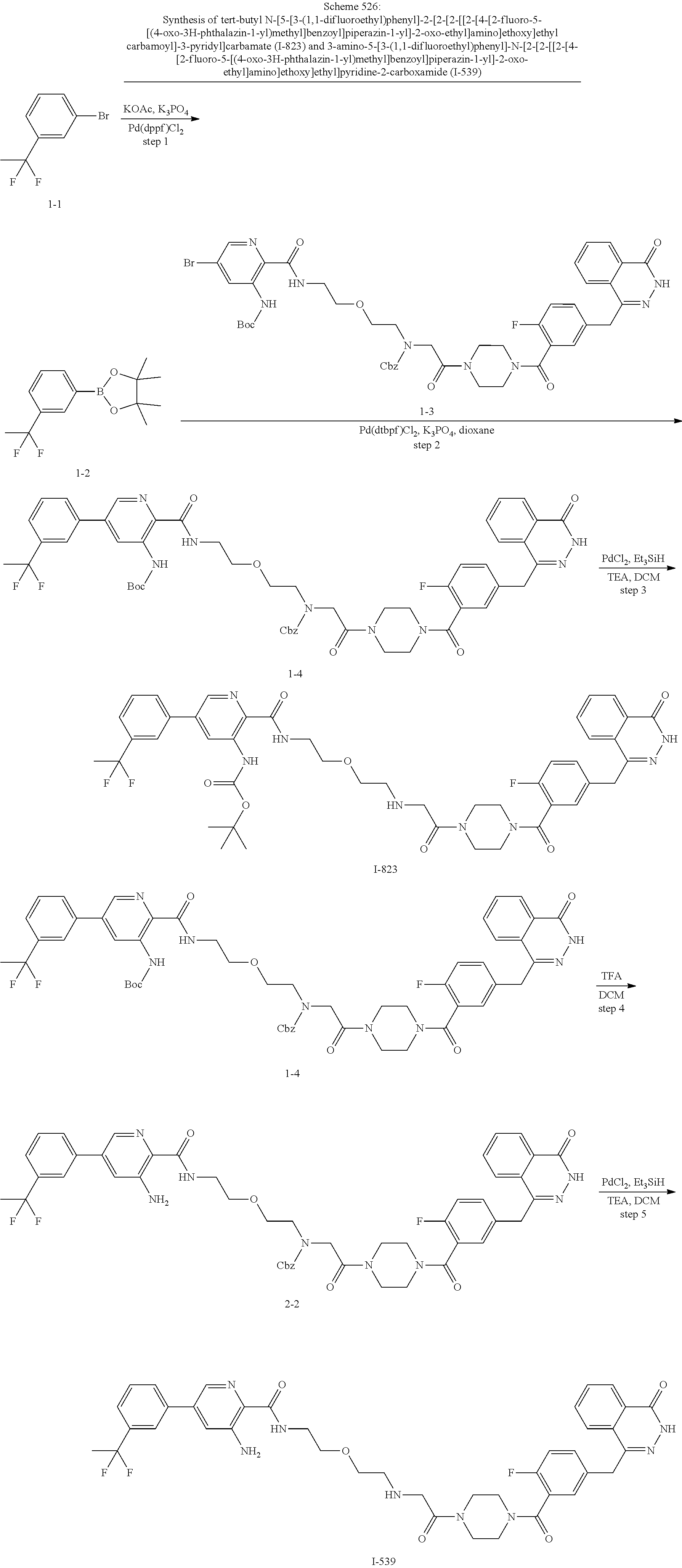

### Step 1: Synthesis of Intermediate 1-2

A mixture of Intermediate 1-1 (1 g, 4.52 mmol, 1 eq), BPD (1.72 g, 6.79 mmol, 1.5 eq), Pd(dppf)Cl₂ (331.02 mg, 452.40 μmol, 0.1 eq) and KOAc (1.33 g, 13.57 mmol, 3 eq) in dioxane (10 mL) was degassed and purged with N₂ 3 times, and then the mixture was stirred at 80° C. for 1 hr under N₂ atmosphere. The reaction mixture was diluted with H₂O (10 mL) and extracted with EA (10 mL*3), dried over Na₂SO₄, filtered and concentrated under reduced pressure to give a residue. The residue was purified by flash silica gel chromatography (ISCO; 12 g SepaFlash Silica Flash Column, Eluent of 0-30% Petroleum ether/Ethyl acetate@40 mL/min). The eluent was concentrated under reduced pressure to give Intermediate 1-2 as a yellow oil. ¹H NMR (400 MHz, CHLOROFORM-d) δ=7.95 (s, 1H), 7.87 (d, J=7.2 Hz, 1H), 7.62 (d, J=7.6 Hz, 1H), 7.44 (t, J=7.2 Hz, 1H), 1.94 (t, J=18.2 Hz, 3H), 1.37 (s, 12H).

### Step 2: Synthesis of Intermediate 1-4

A mixture of Intermediate 1-3 (300 mg, 317.86 μmol, 1 eq), Intermediate 1-2 (102.26 mg, 381.43 μmol, 1.2 eq), K₃PO₄ (202.41 mg, 953.58 μmol, 3 eq) and Pd(dtbpf)Cl₂, (20.72 mg, 31.79 μmol, 0.1 eq) in dioxane (3.5 mL) and H₂O (0.7 mL) was degassed and purged with N₂ 3 times, and then the mixture was stirred at 80° C. for 3 hr under N₂ atmosphere. The reaction mixture was diluted with H₂O (5 mL) and extracted with EA (10 mL*4), dried over Na₂SO₄, filtered and concentrated under reduced pressure to give a residue. The residue was purified by flash silica gel chromatography (ISCO; 40 g SepaFlash Silica Flash Column, Eluent of 0-100% Dichloromethane/Methanol@60 mL/min). The eluent was concentrated under reduced pressure to give Intermediate 1-4 (300 mg, 298.49 μmol, 93.91% yield) as a brown solid. LCMS (Method D): Rt=0.504 min, [M+H]⁺=1005.2.

### Step 3: Synthesis of I-823

To a solution of Intermediate 1-4 (20 mg, 19.90 μmol, 1 eq) in DCM (0.2 mL) was added PdCl₂ (1.76 mg, 9.95 μmol, 0.5 eq), Et₃SiH (27.77 mg, 238.80 μmol, 38.14 μL, 12 eq) and TEA (6.04 mg, 59.70 μmol, 8.31 μL, 3 eq). The mixture was stirred at 25° C. for 1 hr. The reaction mixture was concentrated under reduced pressure to obtain the crude product. The crude product was diluted with MeOH (5 mL) filtered and concentrated under reduced pressure to give the crude product. The residue was purified by prep-HPLC (column: CD04-Welch Utimate C18 150*25*7 um; mobile phase: [water (FA)-ACN]; gradient: 27%-57% B over 8 min), and the eluent was concentrated and lyophilized to afford I-823 (16.16 mg, 18.56 μmol, 93.24% yield, 100.00% purity) as a white solid. LCMS (Method D): Rt=0.442 min, [M+H]⁺=871.4. ¹H NMR (400 MHz, METHANOL-d₄) δ=9.03 (s, 1H), 8.51 (d, J=7.2 Hz, 1H), 8.38-8.34 (m, 1H), 7.95-7.91 (m, 1H), 7.89-7.85 (m, 1H), 7.85-7.81 (m, 2H), 7.79 (d, J=6.4 Hz, 1H), 7.66-7.57 (m, 2H), 7.52-7.45 (m, 1H), 7.33 (d, J=2.4 Hz, 1H), 7.17-7.10 (m, 1H), 4.37 (d, J=4.4 Hz, 2H), 3.74 (s, 2H), 3.72-3.65 (m, 6H), 3.65-3.59 (m, 3H), 3.53-3.46 (m, 2H), 3.34 (d, J=1.6 Hz, 2H), 3.25 (s, 1H), 3.00-2.93 (m, 2H), 2.04-1.91 (m, 3H), 1.55 (s, 9H).

### Step 4: Synthesis of Intermediate 2-2

To a solution of Intermediate 1-4 (200 mg, 199.00 μmol, 1 eq) in DCM (1.6 mL) was added TFA (1.23 g, 10.77 mmol, 0.8 mL, 54.12 eq). The mixture was stirred at 25° C. for 1 hr. The reaction mixture was concentrated under reduced pressure to give the crude product. The crude product was used in the next step without further purification. Intermediate 2-2 (230 mg, crude, TFA salt) was obtained as a brown oil. LCMS (Method D): Rt=0.445 min, [M+H]⁺=905.2.

### Step 5: Synthesis of I-539

To a solution of Intermediate 2-2 (30 mg, 29.44 μmol, 1 eq, TFA salt) in DCM (0.3 mL) was added PdCl₂ (2.61 mg, 14.72 μmol, 0.5 eq), Et₃SiH (41.08 mg, 353.30 μmol, 56.43 μL, 12 eq) and TEA (8.94 mg, 88.33 μmol, 12.29 μL, 3 eq). The mixture was stirred at 25° C. for 1 hr. The residue was diluted with H₂O (5 mL) and extracted with EA (5 mL*3), dried over Na₂SO₄, filtered and concentrated under reduced pressure to give a residue. The crude product was purified by reversed-phase chromatography (0.1% TFA condition), and was concentrated and lyophilized to afford I-539 (15.84 mg, 17.63 μmol, 59.88% yield, 98.483% purity, TFA salt) as a yellow solid. LCMS (Method D): Rt=0.380 min, (M+H)=771.3. ¹H NMR (400 MHz, METHANOL-d₄) δ=8.38-8.35 (m, 1H), 8.12-8.07 (m, 1H), 7.97-7.91 (m, 1H), 7.89-7.80 (m, 2H), 7.76 (s, 1H), 7.71 (t, J=6.0 Hz, 1H), 7.62-7.55 (m, 2H), 7.51-7.48 (m, 1H), 7.41-7.38 (m, 1H), 7.33 (t, J=6.4 Hz, 1H), 7.17-7.11 (m, 1H), 4.37 (d, J=2.8 Hz, 2H), 4.17-4.07 (m, 2H), 3.82 (d, J=4.0 Hz, 2H), 3.79-3.68 (m, 5H), 3.68-3.65 (m, 1H), 3.67-3.57 (m, 2H), 3.53-3.44 (m, 2H), 3.30-3.25 (m, 4H), 2.02-1.89 (m, 3H). ¹⁹F NMR (376 MHz, METHANOL-d₄), δ=−77.217, −88.805, −120.637.

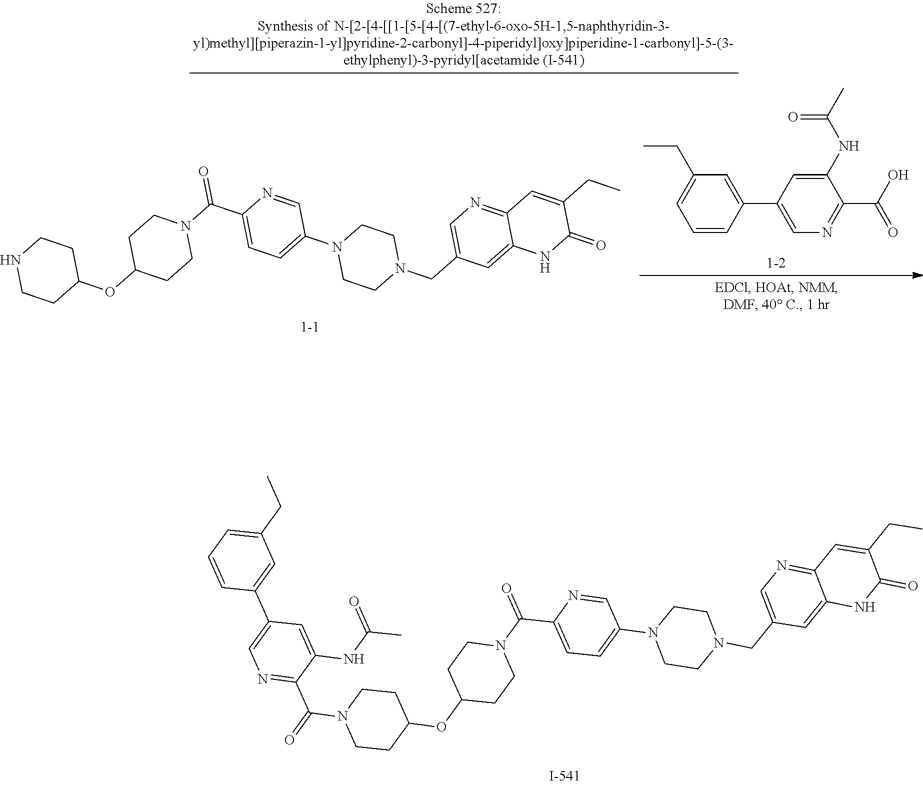 [SMILES: CCc1cc2ncc(CN3CCN(c4ccc(C(=O)N5CCC(OC6CCNCC6)CC5)nc4)CC3)cc2[nH]c1=O ; CCc1cccc(-c2cnc(C(=O)O)c(NC(C)=O)c2)c1 ; CCc1cccc(-c2cnc(C(=O)N3CCC(OC4CCN(C(=O)c5ccc(N6CCN(Cc7cnc8cc(CC)c(=O)[nH]c8c7)CC6)cn5)CC4)CC3)c(NC(C)=O)c2)c1]

To a solution of Intermediate 1-1 (100 mg, 148.43 μmol, 1 eq, TFA salt) in DMF (1 mL) was added Intermediate 1-2 (42.20 mg, 148.43 μmol, 1 eq), EDCI (85.36 mg, 445.29 μmol, 3 eq), HOAt (20.20 mg, 148.43 μmol, 20.76 μL, 1 eq) and NMM (75.07 mg, 742.14 μmol, 81.59 μL, 5 eq). The mixture was stirred at 40° C. for 1 hr. The reaction mixture was diluted with H₂O (4 ml) and extracted with EA (4 mL*2). The combined organic layers were washed with brine (2 mL*1), dried over Na₂SO₄, filtered and concentrated under reduced pressure to give a residue. The crude product was purified by prep-HPLC (column: CD01-Phenomenex luna C18 150*25*10 um; mobile phase: [water (TFA)-ACN]; gradient: 23%-53% B over 10 min). The eluent was concentrated under reduced pressure to remove ACN and then lyophilized to give the product. I-541 (10.27 mg, 12.25 μmol, 8.26% yield, 98.552% purity) was obtained as a white solid. LCMS (Method D): Retention time: 0.371 min, [M+H]⁺=826.3. ¹H NMR (400 MHz, METHANOL-d₄) δ=8.80-8.53 (m, 2H), 8.38-8.29 (m, 1H), 8.27-8.22 (m, 1H), 7.89 (d, J=14.4 Hz, 2H), 7.59-7.46 (m, 4H), 7.46-7.38 (m, 1H), 7.34-7.27 (m, 1H), 4.59 (s, 2H), 4.09-3.95 (m, 2H), 3.89-3.80 (m, 2H), 3.79-3.58 (m, 6H), 3.58-3.44 (m, 6H), 3.43-3.32 (m, 2H), 2.79-2.64 (m, 4H), 2.17 (s, 3H), 2.03-1.82 (m, 4H), 1.77-1.54 (m, 4H), 1.29 (t, J=7.2 Hz, 6H).

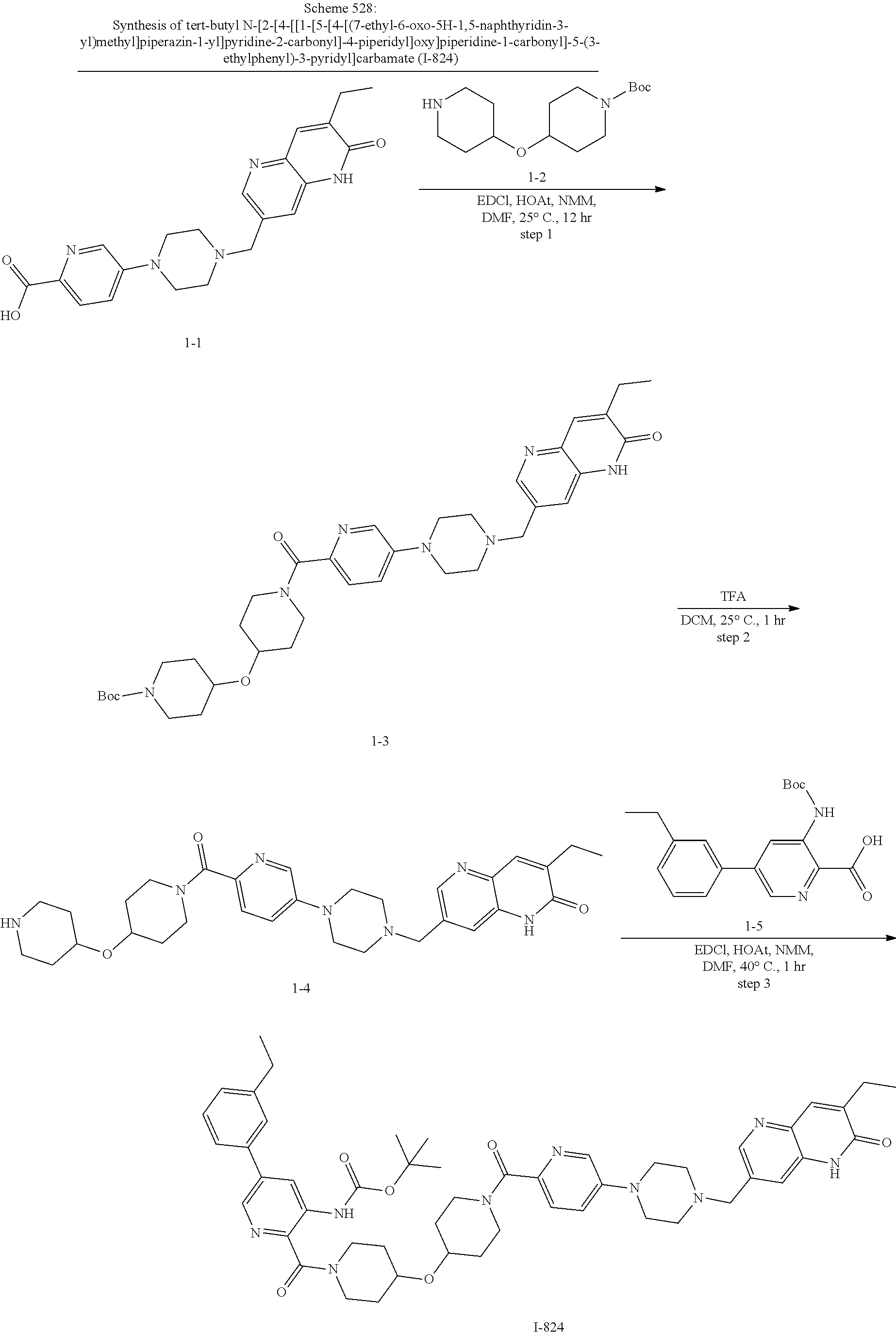 [SMILES: CCc1cc2ncc(CN3CCN(c4ccc(C(=O)O)nc4)CC3)cc2[nH]c1=O ; CC(C)(C)OC(=O)N1CCC(OC2CCNCC2)CC1 ; CCc1cc2ncc(CN3CCN(c4ccc(C(=O)N5CCC(OC6CCN(C(=O)OC(C)(C)C)CC6)CC5)nc4)CC3)cc2[nH]c1=O ; CCc1cc2ncc(CN3CCN(c4ccc(C(=O)N5CCC(OC6CCNCC6)CC5)nc4)CC3)cc2[nH]c1=O ; CCc1cccc(-c2cnc(C(=O)O)c(NC(=O)OC(C)(C)C)c2)c1 ; CCc1cccc(-c2cnc(C(=O)N3CCC(OC4CCN(C(=O)c5ccc(N6CCN(Cc7cnc8cc(CC)c(=O)[nH]c8c7)CC6)cn5)CC4)CC3)c(NC(=O)OC(C)(C)C)c2)c1]

### Step 1: Synthesis of Intermediate 1-3.

To a solution of Intermediate 1-1 (300 mg, 762.51 μmol, 1 eq) in DMF (3 mL) was added Intermediate 1-2 (216.85 mg, 762.51 μmol, 1 eq), EDCI (438.52 mg, 2.29 mmol, 3 eq), HOAt (103.79 mg, 762.51 μmol, 106.67 μL, 1 eq) and NMM (385.63 mg, 3.81 mmol, 419.16 μL, 5 eq). The mixture was stirred at 25° C. for 12 hr. The reaction mixture was diluted with H₂O (3 mL) and extracted with EA (3 mL*2). The combined organic layers were washed with brine (2 mL*1), dried over Na₂SO₄, filtered and concentrated under reduced pressure to give a residue. The residue was purified by flash silica gel chromatography (ISCO; 20 g SepaFlash Silica Flash Column, Eluent of 0˜35% Ethylacetate/Petroleum ether gradient @60 mL/min). The eluent was concentrated under reduced pressure to give product. Intermediate 1-3 (260 mg, 379.21 μmol, 49.73% yield, 96.234% purity) was obtained as a yellow solid. LCMS (Method D): Retention time: 0.343 min, [M+H]⁺=660.3. ¹H NMR (400 MHz, METHANOL-d₄) δ =8.50 (d, J=1.6 Hz, 1H), 8.24 (d, J=2.4 Hz, 1H), 7.84 (s, 1H), 7.77 (s, 1H), 7.51-7.45 (m, 1H), 7.45-7.39 (m, 1H), 3.87-3.78 (m, 2H), 3.75 (s, 3H), 3.72-3.66 (m, 3H), 3.40-3.36 (m, 4H), 3.20-3.07 (m, 5H), 2.70-2.63 (m, 5H), 2.00-1.89 (m, 2H), 1.86-1.81 (m, 4H), 1.69-1.54 (m, 2H), 1.45 (s, 9H), 1.31-1.26 (m, 3H).

### Step 2: Synthesis of Intermediate 1-4.

A solution of Intermediate 1-3 (230 mg, 348.58 μmol, 1 eq) in DCM (2 mL) and TFA (0.4 mL) was stirred at 25° C. for 1 hr. The mixture was concentrated in vacuum to give crude product. Intermediate 1-4 (520 mg, crude, TFA salt) was obtained as a yellow oil, which was used in the next step without further purification. LCMS (Method D): Retention time: 0.220 min, [M+H]⁺=560.2.

### Step 3: Synthesis of I-824

To a solution of Intermediate 1-4 (400 mg, 593.71 μmol, 1 eq, TFA salt) in DMF (4 mL) was added Intermediate 1-5 (203.28 mg, 593.71 μmol, 1 eq), EDCI (341.45 mg, 1.78 mmol, 3 eq), HOAt (80.81 mg, 593.71 μmol, 83.05 μL, 1 eq) and NMM (300.26 mg, 2.97 mmol, 326.37 μL, 5 eq). The mixture was stirred at 40° C. for 1 hr. The reaction mixture was diluted with H₂O (2 mL) and extracted with EA (3 mL*2). The combined organic layers were washed with brine (2 mL*1), dried over Na₂SO₄, filtered and concentrated under reduced pressure to give a residue. The crude product was purified by prep-HPLC (column: CD02-Waters Xbidge BEH C18 150*25*10 um; mobile phase: [water (NH₃H₂O)-ACN]; gradient: 511%-81% B over 14 min). The eluent was concentrated under reduced pressure to remove ACN and then lyophilized to give a residue. Then the residue was purified by reversed-phase chromatography (0.1% NH₃·H₂O condition). The eluent was concentrated under reduced pressure to remove ACN and then lyophilized to give product. I-824 (22.94 mg, 25.82 μmol, 45.65% yield, 99.496% purity) was obtained as a yellow solid. LCMS (Method F): Retention time: 0.727 min, [M+H]⁺=884.7. ¹H NMR (400 MHz, METHANOL-d₄) δ=8.55-8.50 (m, 1H), 8.50-8.46 (m, 1H), 8.43-8.38 (m, 1H), 8.26-8.20 (m, 1H), 7.87-7.80 (m, 1H), 7.78-7.73 (m, 1H), 7.59-7.45 (m, 3H), 7.44-7.35 (m, 2H), 7.32-7.26 (m, 1H), 4.14-3.94 (m, 2H), 3.85-3.79 (m, 2H), 3.74-3.65 (m, 3H), 3.62-3.48 (m, 2H), 3.39-3.33 (m, 5H), 3.32-3.30 (m, 2H), 2.78-2.71 (m, 2H), 2.70-2.62 (m, 6H), 2.05-1.83 (m, 4H), 1.77-1.57 (m, 4H), 1.53 (s, 9H), 1.28 (br t, J=7.2 Hz, 6H).

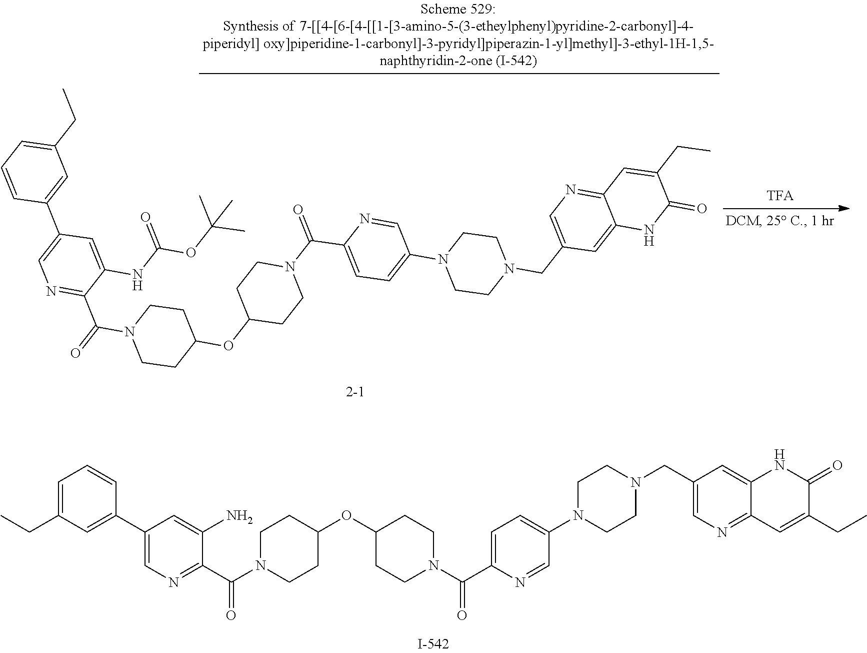

A solution of Intermediate 2-1 (30 mg, 33.93 μmol, 1 eq) in DCM (0.3 mL) and TFA (0.06 mL) was stirred at 25° C. for 1 hr. The mixture was concentrated in vacuum. The crude product was purified by prep-HPLC (column: CD01-Phenomenex luna C18 150*25*10 um; mobile phase: [water (TFA)-ACN]; gradient: 20%-500% B over 10 min). The eluent was concentrated under reduced pressure to remove ACN and then lyophilized to give product. I-542 (23.38 mg, 29.72 μmol, 87.58% yield, 99.652% purity) was obtained as a purple solid. LCMS (Method D): Retention time: 0.359 min, [M+H]⁺=784.4. ¹H NMR (400 MHz, METHANOL-d₄) δ=8.65-8.60 (m, 1H), 8.36-8.29 (m, 1H), 8.25-8.19 (m, 1H), 7.97-7.90 (m, 1H), 7.88-7.83 (m, 1H), 7.79-7.74 (m, 1H), 7.59-7.39 (m, 5H), 7.37-7.30 (m, J=7.3 Hz, 1H), 4.69-4.53 (m, 2H), 4.06-3.96 (m, 1H), 3.90-3.79 (m, 3H), 3.78-3.60 (m, 5H), 3.59-3.47 (m, 6H), 3.43-3.35 (m, 1H), 3.35-3.26 (m, 2H), 2.81-2.61 (m, 4H), 2.06-1.81 (m, 4H), 1.77-1.52 (m, 4H), 1.28 (t, J=7.2 Hz, 6H). ¹⁹FNMR (376 MHz, MeOD-d₆) δ=−77.157.

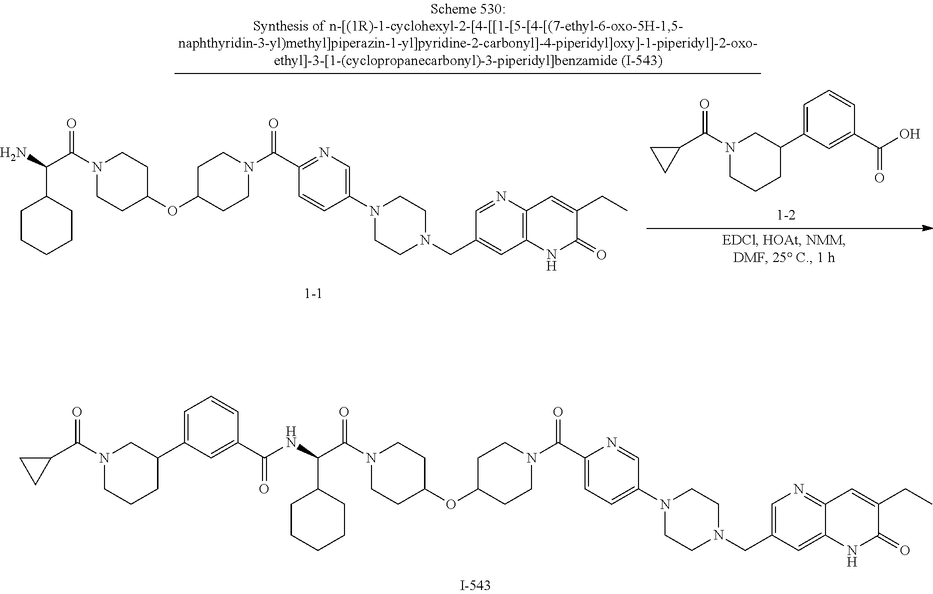 [SMILES: CCc1cc2ncc(CN3CCN(c4ccc(C(=O)N5CCC(OC6CCN(C(=O)[C@H](N)C7CCCCC7)CC6)CC5)nc4)CC3)cc2[nH]c1=O ; O=C(O)c1cccc(C2CCCN(C(=O)C3CC3)C2)c1 ; CCc1cc2ncc(CN3CCN(c4ccc(C(=O)N5CCC(OC6CCN(C(=O)[C@H](NC(=O)c7cccc(C8CCCN(C(=O)C9CC9)C8)c7)C7CCCCC7)CC6)CC5)nc4)CC3)cc2[nH]c1=O]

To a solution of Intermediate 1-1 (17 mg, 20.91 μmol, 1 eq, TFA salt) and Intermediate 1-2 (5.72 mg, 20.91 μmol, 1 eq) in DMF (0.5 mL) was added EDCI (12.03 mg, 62.74 μmol, 3 eq), HOAt (2.85 mg, 20.91 μmol, 2.93 μL, 1 eq), and NMM (10.58 mg, 104.56 μmol, 11.50 μL, 5 eq). The mixture was stirred at 25° C. for 0.5 hr. The mixture was diluted with water (5 mL) and extracted with DCM (5 mL*3). The combined organic layer was dried over Na₂SO₄, filtered and concentrated in vacuo to give a residue. The residue was purified by reverse phase chromatography (0.1% TFA condition), then the residue was concentrated in vacuo and lyophilized. I-543 (6.14 mg, 5.75 μmol, 27.49% yield, TFA salt) was obtained as a white solid. LCMS (Method D): Retention time=0.408 min, [M+H]⁺=954.8. SFC: Retention time=3.635 min, 4.540 min. ¹H NMR (400 MHz, METHANOL-d₄) δ=8.50 (s, 1H), 8.34-8.27 (m, 1H), 8.24 (s, 1H), 7.84 (s, 1H), 7.77 (s, 2H), 7.70 (d, J=4.0 Hz, 1H), 7.47 (d, J=1.6 Hz, 2H), 7.44 (s, 2H), 4.96-4.91 (m, 1H), 4.57 (d, J=10.4 Hz, 1H), 4.43-4.35 (m, 1H), 4.09-3.98 (m, 2H), 3.81 (s, 3H), 3.75 (s, 2H), 3.69-3.57 (m, 1H), 3.54-3.46 (m, 2H), 3.38 (s, 5H), 3.27-3.21 (m, 1H), 3.14 (s, 1H), 2.79-2.73 (m, 1H), 2.72-2.64 (m, 7H), 2.06 (d, J=13.6 Hz, 1H), 1.95-1.84 (m, 7H), 1.81-1.75 (m, 3H), 1.69 (d, J=10.4 Hz, 4H), 1.62-1.52 (m, 3H), 1.29 (t, J=7.2 Hz, 6H), 1.22-1.04 (m, 3H), 0.92-0.79 (m, 4H). ¹⁹F NMR (376 MHz, METHANOL-d₄), δ=−77.024.

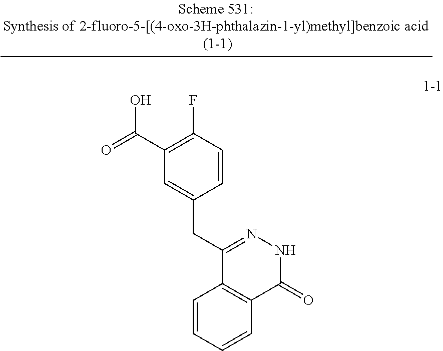 [SMILES: O=C(O)c1cc(Cc2n[nH]c(=O)c3ccccc23)ccc1F]

### Synthesis of 1-1

Intermediate 1-1 was prepared (29.31 mg, 96.20 μmol, 57.39% yield, 97.896% purity) and was obtained as a white solid. LCMS: Rt=0.415 min, (M+H)=299.0. ¹H NMR (400 MHz, DMSO-d₆) δ=13.75-12.95 (m, 1H), 12.59 (s, 1H), 8.26-8.24 (m, 1H), 8.01-7.94 (m, 1H), 7.89-7.88 (m, 1H), 7.85-7.79 (m, 2H), 7.62-7.54 (m, 1H), 7.23-7.22 (m, 1H), 4.35 (s, 2H)

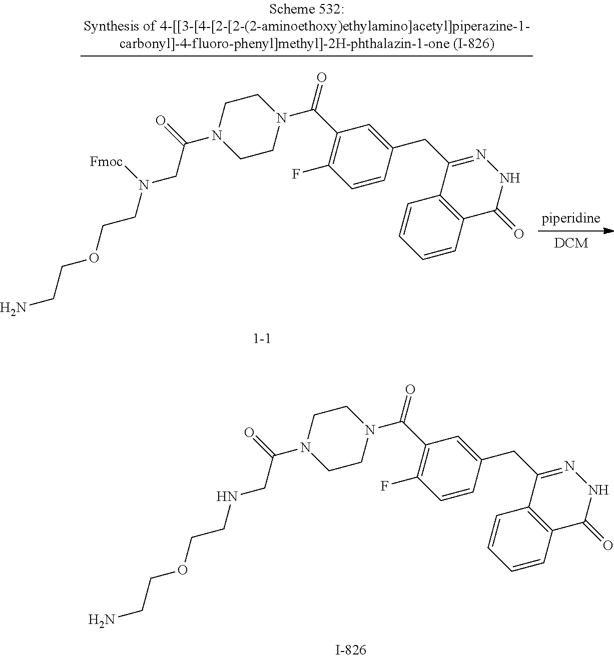

To a solution of Intermediate 1-1 (100 mg, 136.46 μmol, 1 eq) in DCM (1 mL) was added piperidine (116.20 mg, 1.36 mmol, 134.77 μL, 10 eq). The mixture was stirred at 25° C. for 1 h. The mixture was concentrated under vacuum to give a residue. The residue was purified by Prep-HPLC (column: Phenomenex luna C18 150*25 mm*10 um; mobile phase: [water (FA)-ACN]; gradient: 2%-32% B over 9 min). I-826 (22.77 mg, 44.60 μmol, 32.68% yield, 100% purity, FA salt) was obtained as a white solid. LCMS (Method E): Rt=0.348 min, (M+H)=511.3. ¹H NMR (400 MHz, METHANOL-d₄) δ=8.41-8.33 (m, 2H), 7.99-7.93 (m, 1H), 7.92-7.81 (m, 2H), 7.51-7.49 (m, 1H), 7.40-7.36 (m, 1H), 7.19-7.14 (m, 1H), 4.39 (s, 2H), 4.16-4.04 (m, 2H), 3.85-3.69 (m, 7H), 3.56 (br s, 2H), 3.40 (br s, 2H), 3.25 (br d, J=4.4 Hz, 2H), 3.19-3.17 (m, 2H).

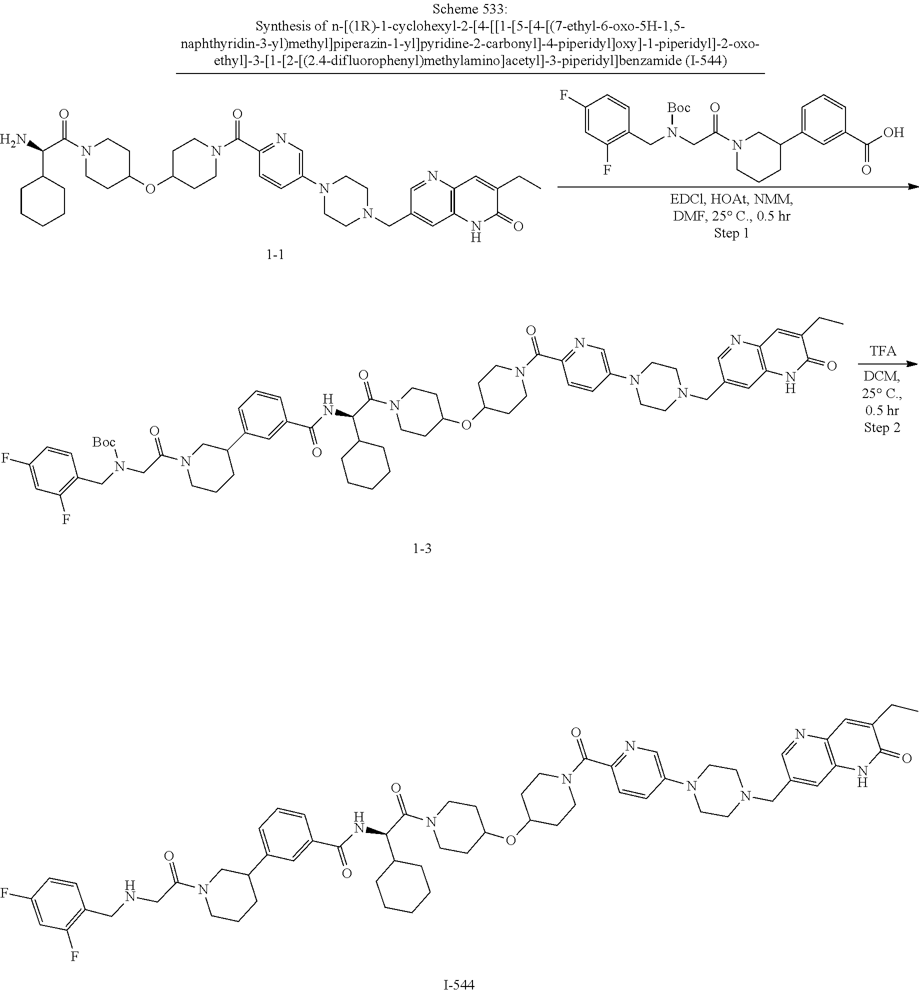 [SMILES: CCc1cc2ncc(CN3CCN(c4ccc(C(=O)N5CCC(OC6CCN(C(=O)[C@H](N)C7CCCCC7)CC6)CC5)nc4)CC3)cc2[nH]c1=O ; CC(C)(C)OC(=O)N(CC(=O)N1CCCC(c2cccc(C(=O)O)c2)C1)Cc1ccc(F)cc1F ; CCc1cc2ncc(CN3CCN(c4ccc(C(=O)N5CCC(OC6CCN(C(=O)[C@H](NC(=O)c7cccc(C8CCCN(C(=O)CN(Cc9ccc(F)cc9F)C(=O)OC(C)(C)C)C8)c7)C7CCCCC7)CC6)CC5)nc4)CC3)cc2[nH]c1=O ; CCc1cc2ncc(CN3CCN(c4ccc(C(=O)N5CCC(OC6CCN(C(=O)[C@H](NC(=O)c7cccc(C8CCCN(C(=O)CNCc9ccc(F)cc9F)C8)c7)C7CCCCC7)CC6)CC5)nc4)CC3)cc2[nH]c1=O]

### Step 1: Synthesis of Intermediate 1-3

To a solution of Intermediate 1-1 (70 mg, 86.11 μmol, 1 eq, TFA salt) and Intermediate 1-2 (46.27 mg, 94.72 μmol, 1.1 eq) in DMF (1 mL) was added EDCI (49.52 mg, 258.33 μmol, 3 eq), HOAt (11.72 mg, 86.11 μmol, 12.05 μL, 1 eq), NMM (43.55 mg, 430.55 μmol, 47.34 μL, 5 eq). The mixture was stirred at 25° C. for 0.5 hr. The mixture was diluted with water (5 mL) and extracted with DCM (5 mL*3). The combined organic layer was dried over Na₂SO₄, filtered and concentrated in vacuo to give a residue. The residue was purified by prep-HPLC (column: CD01-Phenomenex luna C18 150*25*10 um; mobile phase: [water (TFA)-ACN]; gradient: 40%-70% B over 10 min). The eluent was concentrated in vacuo and lyophilized to give product. Intermediate 1-3 (50 mg, 41.47 μmol, 48.16% yield, 97% purity) was obtained as a white solid. LCMS (Method D): Retention time=0.438 min, [M+H]⁺=1169.5. SFC: Retention time=2.225 min, 2.438 min.

### Step 2: Synthesis of I-544

To a solution of Intermediate 1-3 (50 mg, 42.76 μmol, 1 eq) in DCM (0.5 mL) was added TFA (153.50 mg, 1.35 mmol, 0.1 mL, 31.49 eq). The mixture was stirred at 25° C. for 0.5 hr. The mixture was diluted with water (5 mL) and extracted with DCM (5 mL*3). The combined organic layer was dried over Na₂SO₄, filtered and concentrated in vacuo to give a residue. The residue was purified by prep-HPLC (column: CD07-Daisogel SP-100-8-ODS-PK 150*25*10 um; mobile phase: [water (NH₄HCO₃)-ACN]; gradient: 35%-65% B over 10 min). Then the eluent was concentrated in vacuo and lyophilized to give product. I-544 (9.21 mg, 7.78 μmol, 18.20% yield, 100% purity, TFA salt) was obtained as a white solid. LCMS (Method D): Retention time=0.363 min, [M+H]⁺=1069.4. SFC: Retention time=2.705 min, 2.970 min. ¹H NMR (400 MHz, METHANOL-d4) δ=8.49 (s, 1H), 8.24 (s, 1H), 7.84 (s, 1H), 7.78-7.69 (m, 3H), 7.50-7.40 (m, 5H), 7.01-6.87 (m, 2H), 4.96-4.93 (m, 2H), 4.58-4.53 (m, 1H), 4.03 (d, J=5.2 Hz, 2H), 3.98-3.86 (m, 2H), 3.82 (d, J=9.6 Hz, 5H), 3.74 (s, 3H), 3.53 (s, 2H), 3.49 (d, J=8.0 Hz, 2H), 3.38 (d, J=1.2 Hz, 4H), 3.20-3.06 (m, 2H), 2.71-2.65 (m, 6H), 2.03 (d, J=1.6 Hz, 1H), 1.95-1.90 (m, 2H), 1.90-1.83 (m, 5H), 1.78-1.76 (m, 2H), 1.73-1.64 (m, 4H), 1.63-1.48 (m, 4H), 1.29 (t, J=7.2 Hz, 7H), 1.15-1.07 (m, 2H). ¹⁹F NMR (376 MHz, METHANOL-d₄), δ=−77.144, −108.380, −112.864.

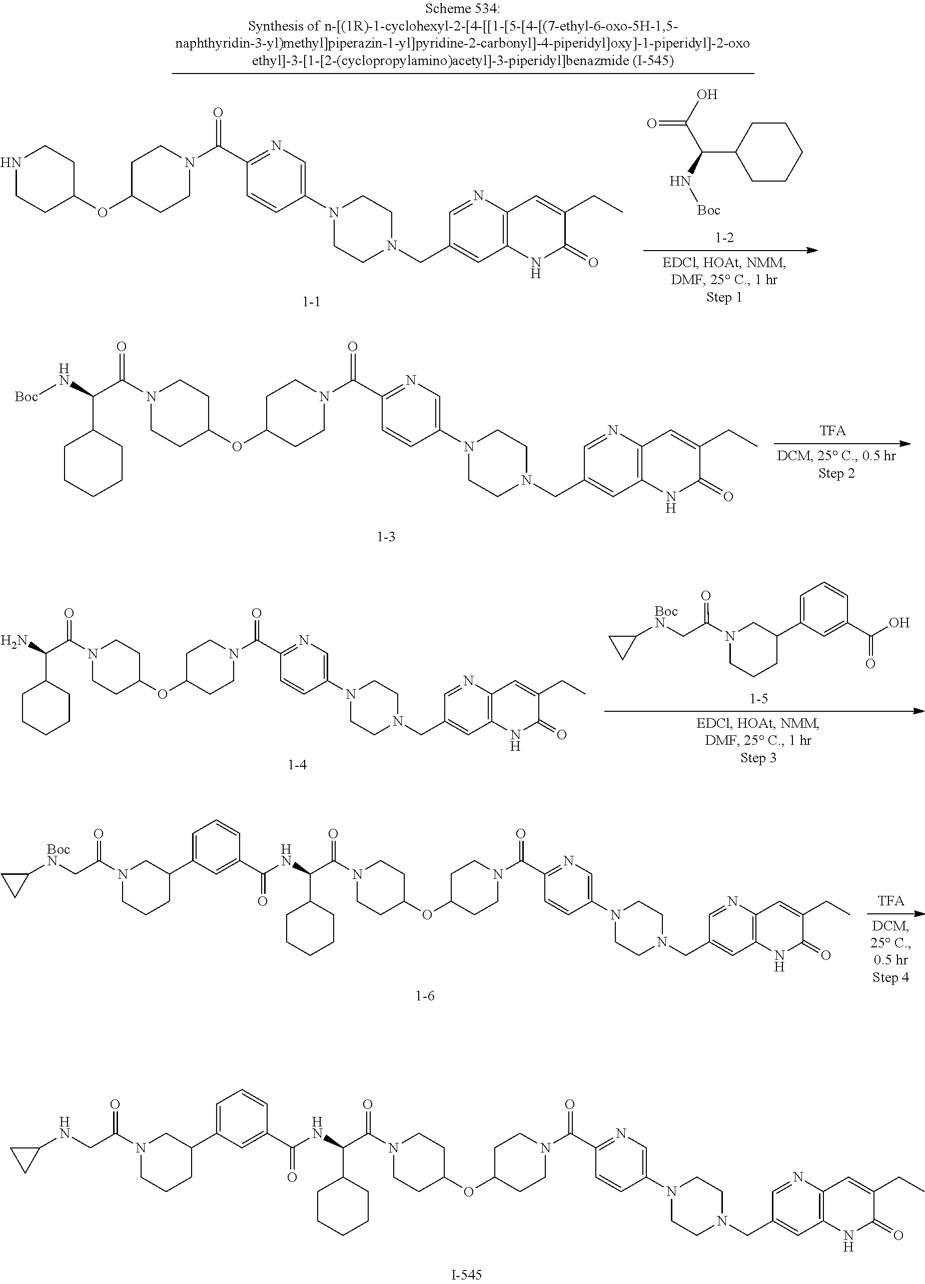

### Step 1: Synthesis of Intermediate 1-3

To a solution of Intermediate 1-1 (350 mg, 519.50 μmol, 1 eq, TFA salt) and Intermediate 1-2 (160.42 mg, 623.40 μmol, 1.2 eq) in DMF (3 mL) was added EDCI (298.77 mg, 1.56 mmol, 3 eq), HOAt (70.71 mg, 519.50 μmol, 72.67 μL, 1 eq), and NMM (262.73 mg, 2.60 mmol, 285.58 μL, 5 eq). The mixture was stirred at 25° C. for 1 hr. The mixture was diluted with water (5 mL) and extracted with DCM (5 mL*3). The combined organic layer was dried over Na₂SO₄, filtered and concentrated in vacuo to give a residue. The residue was purified by reverse phase chromatography (neutral condition), then the residue was concentrated in vacuo and lyophilized to give product. Intermediate 1-3 (250 mg, 272.21 μmol, 52.40% yield, 87% purity) was obtained as a white solid. LCMS (Method D): Retention time=0.384 min, [M+H]⁺=799.7.

### Step 2: Synthesis of Intermediate 1-4

To a solution of Intermediate 1-3 (140 mg, 175.22 μmol, 1 eq) in DCM (1.2 mL) was added TFA (614.00 mg, 5.38 mmol, 0.4 mL, 30.73 eq). The mixture was stirred at 25° C. for 0.5 hr. The reaction mixture was concentrated in vacuo to give a residue. Intermediate 1-4 (140 mg, crude, TFA salt) was obtained as a yellow oil and used in the next step directly without further purification. LCMS (Method D): Retention time=0.274 min, [M+H]⁺=699.7.

### Step 3: Synthesis of Intermediate 1-6

To a solution of Intermediate 1-4 (70 mg, 86.11 μmol, 1 eq, TFA salt) and Intermediate 1-5 (38.12 mg, 94.72 μmol, 1.1 eq) in DMF (1 mL) was added EDCI (49.52 mg, 258.33 μmol, 3 eq), HOAt (11.72 mg, 86.11 μmol, 12.05 μL, 1 eq), and NMM (43.55 mg, 430.55 μmol, 47.34 μL, 5 eq). The mixture was stirred at 25° C. for 0.5 hr. The mixture was diluted with water (5 mL) and extracted with DCM (5 mL*3). The combined organic layer was dried over Na₂SO₄, filtered and concentrated in vacuo to give a residue. The mixture was purified by prep-HPLC (column: CD01-Phenomenex luna C18 150*25*10 um; mobile phase: [water (TFA)-ACN]; gradient: 38%-68% B over 10 min). The eluent was concentrated in vacuo and lyophilized to give product. Intermediate 1-6 (50 mg, 45.23 μmol, 52.53% yield, 98% purity) was obtained as a white solid. LCMS (Method D): Retention time=0.417 min, [M+H]⁺=1084.5. SFC: Retention time=2.721 min, 3.108 min.

### Step 4: Synthesis of I-545

To a solution of Intermediate 1-6 (50 mg, 46.15 μmol, 1 eq) in DCM (0.5 mL) was added TFA (153.50 mg, 1.35 mmol, 0.1 mL, 29.17 eq). The mixture was stirred at 25° C. for 0.5 hr. The reaction mixture was concentrated in vacuo to give a residue. The residue was purified by reverse phase chromatography (0.1% TFA condition), then the eluent was concentrated in vacuo and lyophilized to give product. I-545 (36.3 mg, 31.53 μmol, 68.33% yield, 95.321% purity, TFA salt) was obtained as a yellow solid. LCMS (Method D): Retention time=0.340 min, [M+H]⁺=983.4. SFC: Retention time=2.712 min, 3.025 min. ¹H NMR (400 MHz, METHANOL-d₄) δ=8.63 (s, 1H), 8.34 (d, J=3.6 Hz, 1H), 7.94 (s, 1H), 7.88 (s, 1H), 7.79 (s, 1H), 7.75-7.70 (m, 1H), 7.60-7.52 (m, 2H), 7.51-7.40 (m, 2H), 4.96 (d, J=8.4 Hz, 1H), 4.60 (s, 2H), 4.56-4.42 (m, 1H), 4.31-4.15 (m, 2H), 4.12-3.91 (m, 3H), 3.85-3.75 (m, 4H), 3.66 (s, 4H), 3.53 (s, 6H), 3.40-3.34 (m, 1H), 3.29-3.16 (m, 2H), 3.16-2.99 (m, 1H), 2.94-2.84 (m, 1H), 2.82-2.74 (m, 2H), 2.69-2.66 (m, 2H), 2.07 (d, J=11.6 Hz, 1H), 1.94 (s, 2H), 1.87 (d, J=11.2 Hz, 4H), 1.78 (d, J=9.6 Hz, 2H), 1.69 (d, J=12.0 Hz, 4H), 1.63-1.52 (m, 3H), 1.29 (t, J=7.6 Hz, 5H), 1.26-1.17 (m, 2H), 1.11 (d, J=9.6 Hz, 2H), 0.94-0.88 (m, 4H). ¹⁹F NMR (376 MHz, METHANOL-d₄), δ=−77.427.

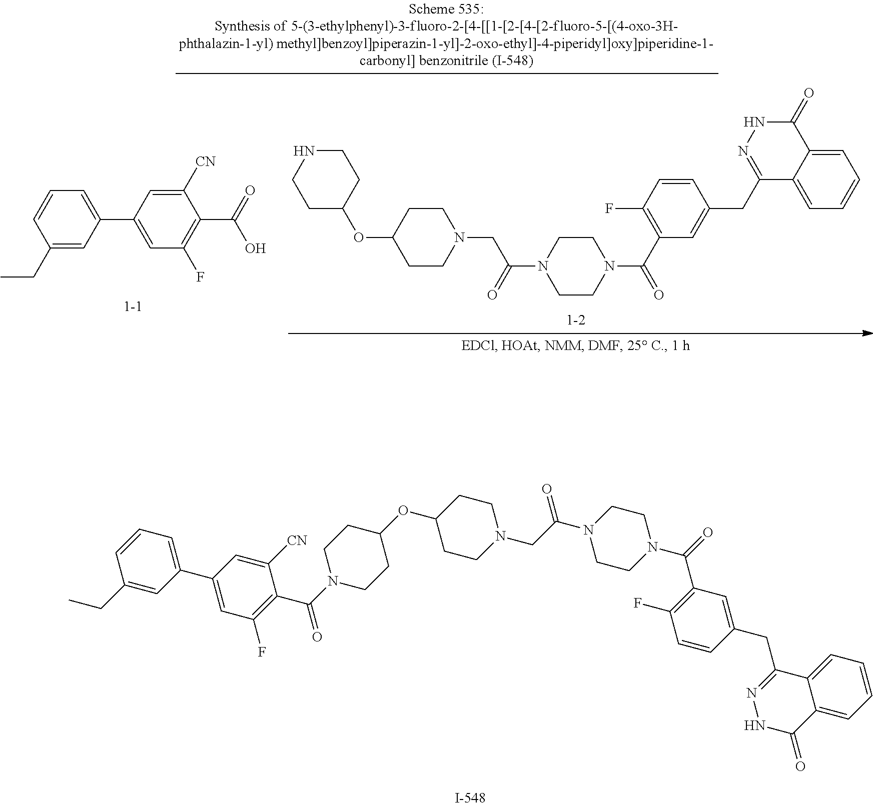

To a solution of Intermediate 1-1 (58.69 mg, 93.59 μmol, 1.2 eq, HCl salt) in DMF (0.5 mL) was added EDCI (44.85 mg, 233.97 μmol, 3 eq), HOAt (10.62 mg, 77.99 μmol, 10.91 μL, 1 eq), and NMM (39.44 mg, 389.94 μmol, 42.87 μL, 5 eq). The mixture was stirred at 25° C. for 0.5 hr. The mixture was diluted with water (5 mL) and extracted with DCM (5 mL*3). The combined organic layer was dried over Na₂SO₄, filtered and concentrated in vacuo to give a residue. The residue was purified by prep-HPLC (column: CD01-Phenomenex luna C18 150*25*10 um; mobile phase: [water (TFA)-ACN]; gradient: 28%-58% B over 10 min). The eluent was concentrated in vacuo and lyophilized to give product. I-548 (16.59 mg, 17.28 μmol, 22.16% yield, 99.564% purity, TFA salt) was obtained as a white solid. LCMS (Method D): Retention time=0.408 min, [M+H]⁺=842.5. ¹H NMR (400 MHz, METHANOL-d₄) δ=8.37 (d, J=7.2 Hz, 1H), 8.00 (s, 1H), 7.97-7.93 (m, 1H), 7.90-7.82 (m, 3H), 7.56 (s, 1H), 7.52 (d, J=7.2 Hz, 2H), 7.43 (t, J=7.6 Hz, 1H), 7.37 (d, J=5.6 Hz, 1H), 7.34-7.31 (m, 1H), 7.18 (t, J=9.2 Hz, 1H), 4.39 (s, 2H), 4.31 (s, 1H), 4.23 (s, 1H), 4.19-4.02 (m, 1H), 3.96 (s, 2H), 3.83-3.77 (m, 3H), 3.75-3.70 (m, 1H), 3.68-3.62 (m, 1H), 3.56 (s, 2H), 3.53-3.47 (m, 2H), 3.44 (d, J=1.2 Hz, 1H), 3.35 (s, 2H), 3.21-3.04 (m, 2H), 2.78-2.72 (m, 2H), 2.30-2.14 (m, 1H), 2.11-1.99 (m, 4H), 1.96-1.82 (m, 2H), 1.78-1.64 (m, 2H), 1.29 (t, J=7.6 Hz, 3H). ¹⁹F NMR (377 MHz, METHANOL-d₄), δ=−77.232, −114.932, −120.712.

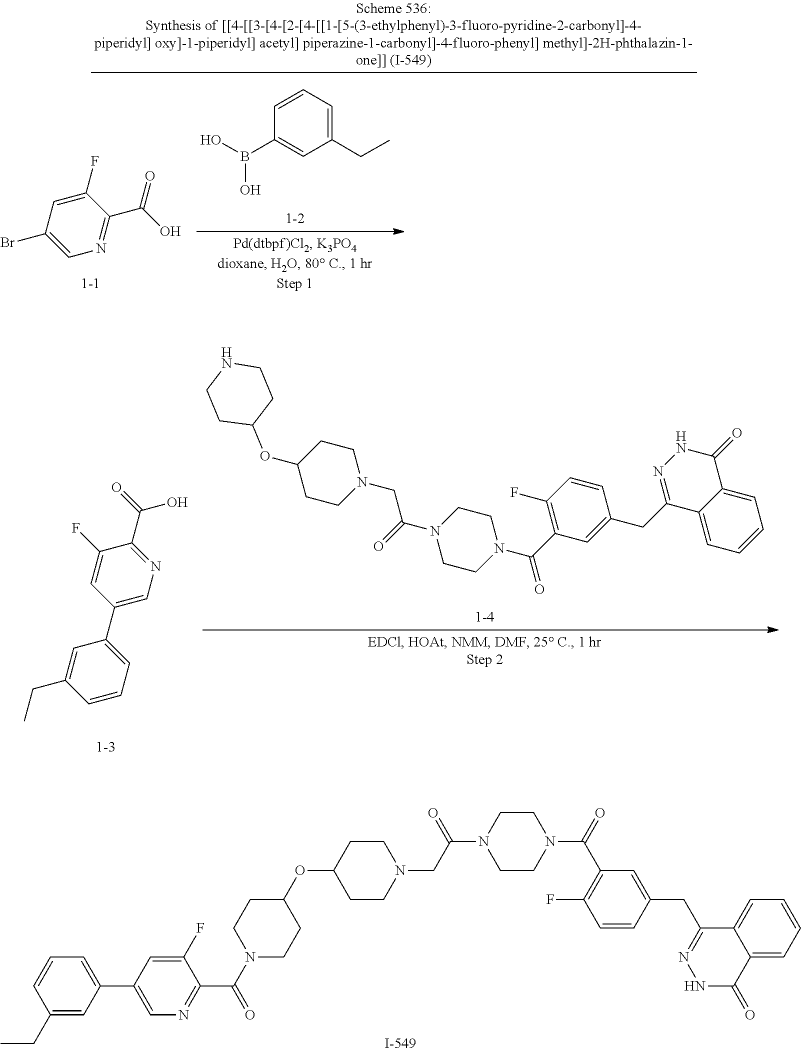 [SMILES: O=C(O)c1ncc(Br)cc1F ; CCc1cccc(B(O)O)c1 ; CCc1cccc(-c2cnc(C(=O)O)c(F)c2)c1 ; O=C(CN1CCC(OC2CCNCC2)CC1)N1CCN(C(=O)c2cc(Cc3n[nH]c(=O)c4ccccc34)ccc2F)CC1 ; CCc1cccc(-c2cnc(C(=O)N3CCC(OC4CCN(CC(=O)N5CCN(C(=O)c6cc(Cc7n[nH]c(=O)c8ccccc78)ccc6F)CC5)CC4)CC3)c(F)c2)c1]

### Step 1: Synthesis of Intermediate 1-3

A mixture of Intermediate 1-1 (100 mg, 454.55 μmol, 1 eq), Intermediate 1-2 (81.81 mg, 545.46 μmol, 1.2 eq), Pd(dtbpf)Cl₂ (29.63 mg, 45.46 μmol, 0.1 eq) and K₃PO₄ (289.46 mg, 1.36 mmol, 3 eq) in dioxane (1 mL) and H₂O (0.2 mL) was degassed and purged with N₂ 3 times, and then the mixture was stirred at 80° C. for 1 hr under N₂ atmosphere. The reaction mixture was poured into H₂O (2 mL) and extracted with EA (2 mL*3). The combined organic layers were dried over Na₂SO₄, filtered and concentrated under reduced pressure to give a residue. The residue was purified by flash silica gel chromatography (ISCO; 12 g SepaFlash Silica Flash Column, Eluent of 0˜100% MeOH/DCM @36 mL/min, DCM/MeOH=10:1, Rf=0.3) and the eluent was concentrated under reduced pressure to give the product. Intermediate 1-3 (60 mg, crude) was obtained as a brown gum. LCMS (Method D): Rt=0.391 min, [M+H]⁺=246.1.

### Step 2: Synthesis of I-549

To a solution of Intermediate 1-3 (40 mg, 163.10 μmol, 1 eq) and Intermediate 1-4 (102.29 mg, 163.10 μmol, 1 eq, HCl salt) in DMF (0.4 mL) was added EDCI (93.80 mg, 489.30 μmol, 3 eq), HOAt (22.20 mg, 163.10 μmol, 22.82 μL, 1 eq) and NMM (82.49 mg, 815.50 μmol, 89.66 μL, 5 eq). The mixture was stirred at 25° C. for 0.5 hr. The reaction mixture was poured into H₂O (2 mL), extracted with EA (2 mL*3). The combined organic layers were dried over Na₂SO₄, filtered and concentrated under reduced pressure to give a residue. The residue was purified by prep-HPLC (column: CD03-Welch Xtimate C18 150*25*5 um; mobile phase: [water (FA)-ACN]; gradient: 21%-51% B over 10 min) and the eluent was concentrated to remove ACN and lyophilized to give product. I-549 (22.73 mg, 26.23 μmol, 16.08% yield, 99.715% purity, FA salt) was obtained as a yellow solid. LCMS (Method D): Rt=0.394 min, [M+H]⁺=818.2. ¹H NMR (400 MHz, METHANOL-d₄) δ=8.73 (s, 1H), 8.45 (s, 1H), 8.41-8.33 (m, 1H), 8.03-8.00 (m, 1H), 7.97-7.92 (m, 1H), 7.90-7.80 (m, 2H), 7.59-7.47 (m, 3H), 7.44 (t, J=7.6 Hz, 1H), 7.40-7.31 (m, 2H), 7.17 (t, J=9.2 Hz, 1H), 4.39 (s, 2H), 4.12-3.99 (m, 1H), 3.88-3.79 (m, 2H), 3.75 (d, J=5.2 Hz, 1H), 3.73-3.66 (m, 3H), 3.65-3.57 (m, 3H), 3.54 (d, J=4.4 Hz, 2H), 3.47 (d, J=4.4 Hz, 1H), 3.38-3.33 (m, 1H), 3.29-3.14 (m, 2H), 3.13-3.00 (m, 2H), 2.75 (q, J=7.6 Hz, 3H), 2.03-1.94 (m, 3H), 1.90-1.83 (m, 1H), 1.82-1.65 (m, 3H), 1.62-1.59 (m, 1H), 1.34-1.25 (m, 4H). ¹⁹F NMR (376 MHz, METHANOL-d₄) δ=−120.772, −127.167.

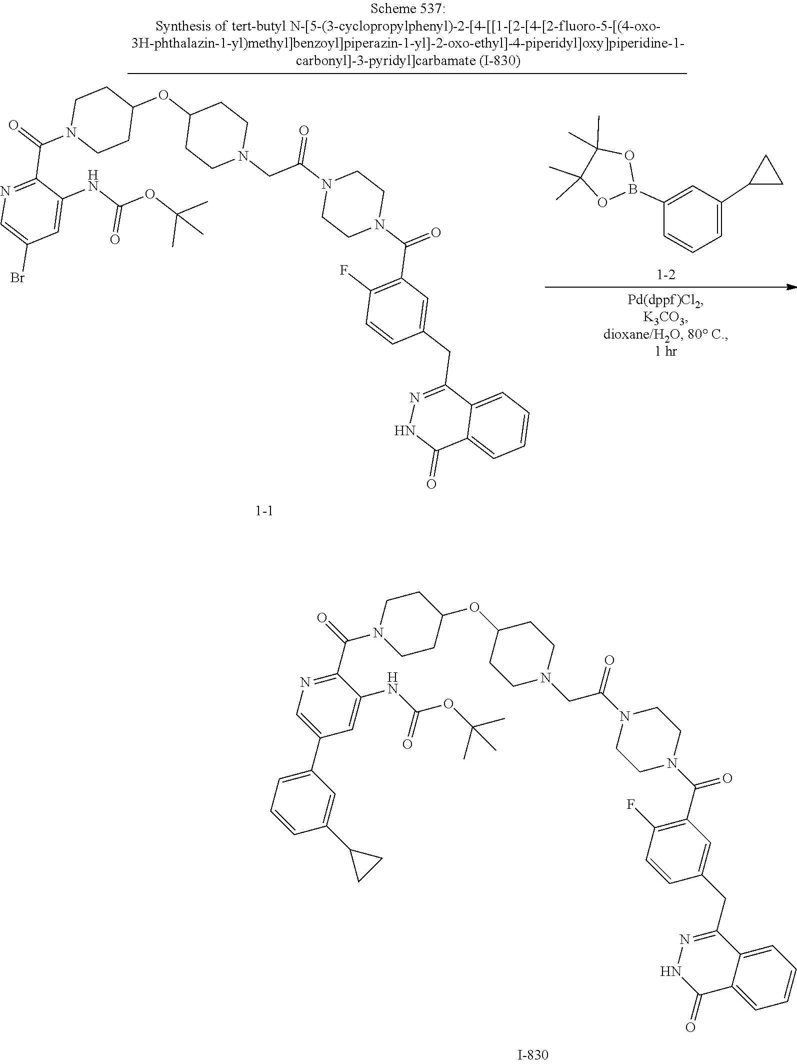 [SMILES: CC(C)(C)OC(=O)Nc1cc(Br)cnc1C(=O)N1CCC(OC2CCN(CC(=O)N3CCN(C(=O)c4cc(Cc5n[nH]c(=O)c6ccccc56)ccc4F)CC3)CC2)CC1 ; CC1(C)OB(c2cccc(C3CC3)c2)OC1(C)C ; CC(C)(C)OC(=O)Nc1cc(-c2cccc(C3CC3)c2)cnc1C(=O)N1CCC(OC2CCN(CC(=O)N3CCN(C(=O)c4cc(Cc5n[nH]c(=O)c6ccccc56)ccc4F)CC3)CC2)CC1]

To a solution of Intermediate 1-1 (50 mg, 56.19 μmol, 1 eq) and Intermediate 1-2 (13.72 mg, 56.19 μmol, 1 eq) in H₂O (0.15 mL) and dioxane (0.45 mL) was added Pd(dppf)Cl₂ (4.11 mg, 5.62 mol, 0.1 eq) and K₂CO₃ (23.30 mg, 168.58 μmol, 3 eq). The suspension was degassed and purged with N₂ 3 times and stirred at 90° C. for 1 hr under N₂. The reaction mixture was diluted with water (2 mL) and extracted with ethyl acetate (3 mL*3). The combined organic layers were washed with brine (4 mL*3) and dried over sodium sulfate, filtered and concentrated under reduced pressure to give a residue. The crude product was purified by reversed-phase chromatography (neutral condition) and the eluent was concentrated under reduced pressure to remove ACN and then lyophilized to give I-830 (22.06 mg, 23.80 μmol, 42.35% yield, 100% purity) as a white solid. LCMS (Method D): Rt: 0.441 min, [M+H]⁺=927.2. ¹H NMR (400 MHz, METHANOL-d₄) δ=8.52 (d, J=1.6 Hz, 1H), 8.40-8.34 (m, 2H), 7.99-7.92 (m, 1H), 7.90-7.80 (m, 2H), 7.52-7.42 (m, 2H), 7.41-7.35 (m, 3H), 7.19-7.12 (m, 2H), 4.39 (s, 2H), 4.11-4.00 (m, 1H), 3.83-3.64 (m, 7H), 3.58-3.50 (m, 4H), 3.26 (bs, 1H), 3.19 (s, 1H), 2.82-2.71 (m, 2H), 2.33-2.21 (m, 2H), 2.06-1.95 (m, 2H), 1.88 (s, 4H), 1.74-1.59 (m, 4H), 1.53 (d, J=1.6 Hz, 10H), 1.06-0.98 (m, 2H), 0.81-0.73 (m, 2H). ¹⁹F NMR (376 MHz, METHANOL-d₄) δ=−120.794.

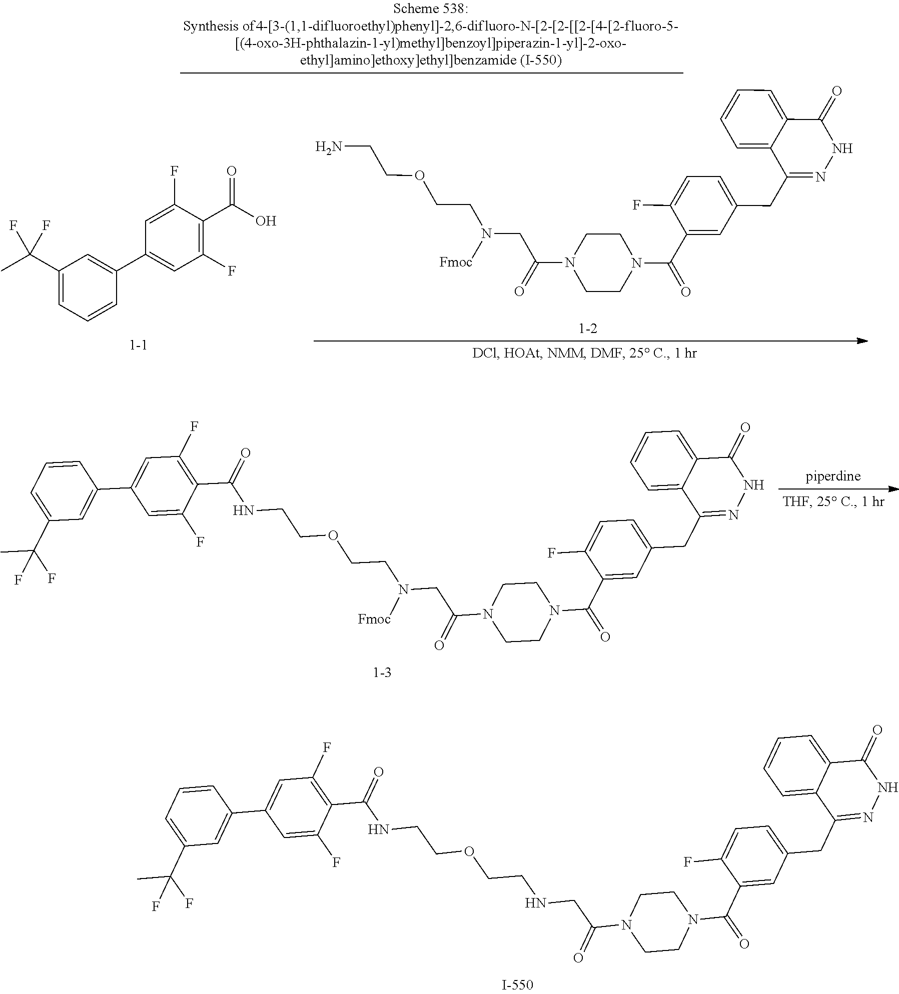

### Step 1: Synthesis of Intermediate 1-3

To a solution of Intermediate 1-1 (50 mg, 167.65 μmol, 1 eq) in DMF (1 mL) was added HOAt (22.82 mg, 167.65 μmol, 23.45 μL, 1 eq), EDCI (96.42 mg, 502.96 μmol, 3 eq), NMM (84.79 mg, 838.27 μmol, 92.16 μL, 5 eq) and Intermediate 1-2 (141.87 mg, 184.42 μmol, 1.1 eq, HCl salt). The mixture was stirred at 25° C. for 1 hr. The residue was diluted with H₂O (5 ml) and extracted with EA (5 mL*3), dried over Na₂SO₄, filtered and concentrated under reduced pressure to give a residue. The combined crude product was purified by flash silica gel chromatography (ISCO; 12 g SepaFlash Silica Flash Column, Eluent of 0-100% Methanol/Dichloromethane @30 mL/min). The eluent was concentrated under reduced pressure to give Intermediate 1-3 (150 mg, 148.07 μmol, 88.32% yield) as a yellow solid. LCMS (Method D): Rt=0.485 min, [M+H]⁺=1013.3.

### Step 2: Synthesis of I-550

To a solution of Intermediate 1-3 (50 mg, 49.36 μmol, 1 eq) in THF (0.5 mL) was added piperidine (86.22 mg, 1.01 mmol, 0.1 mL, 20.52 eq). The mixture was stirred at 25° C. for 1 hr. The reaction mixture was concentrated under reduced pressure to obtain the crude product. The combined crude product was purified by reversed-phase chromatography (0.1% NH₃·H₂O condition). The eluent was concentrated in vacuum to remove ACN and lyophilized. Then the crude product was purified by reversed-phase chromatography (neutral condition), which was concentrated and lyophilized to afford I-550 (38.41 mg, 47.85 μmol, 96.94% yield, 98.504% purity) as a white solid. LCMS (Method H): Rt=0.618 min, [M+H]⁺=791.4. ¹H NMR (400 MHz, METHANOL-d₄) δ=8.36 (d, J=8.0 Hz, 1H), 7.96-7.91 (m, 1H), 7.89-7.81 (m, 2H), 7.78 (s, 1H), 7.74 (s, 1H), 7.62-7.53 (m, 2H), 7.50-7.44 (m, 1H), 7.39 (s, 1H), 7.37-7.31 (m, 2H), 7.13 (t, J=8.8 Hz, 1H), 4.37 (s, 2H), 3.78-3.69 (m, 2H), 3.63 (d, J=7.6 Hz, 8H), 3.55 (s, 2H), 3.48 (s, 2H), 3.38 (d, J=3.2 Hz, 1H), 3.27-3.21 (m, 1H), 2.81 (d, J=4.0 Hz, 2H), 2.01-1.91 (m, 3H). ¹⁹F NMR (377 MHz, METHANOL-d₄) δ=−88.762, −114.616, −120.725.

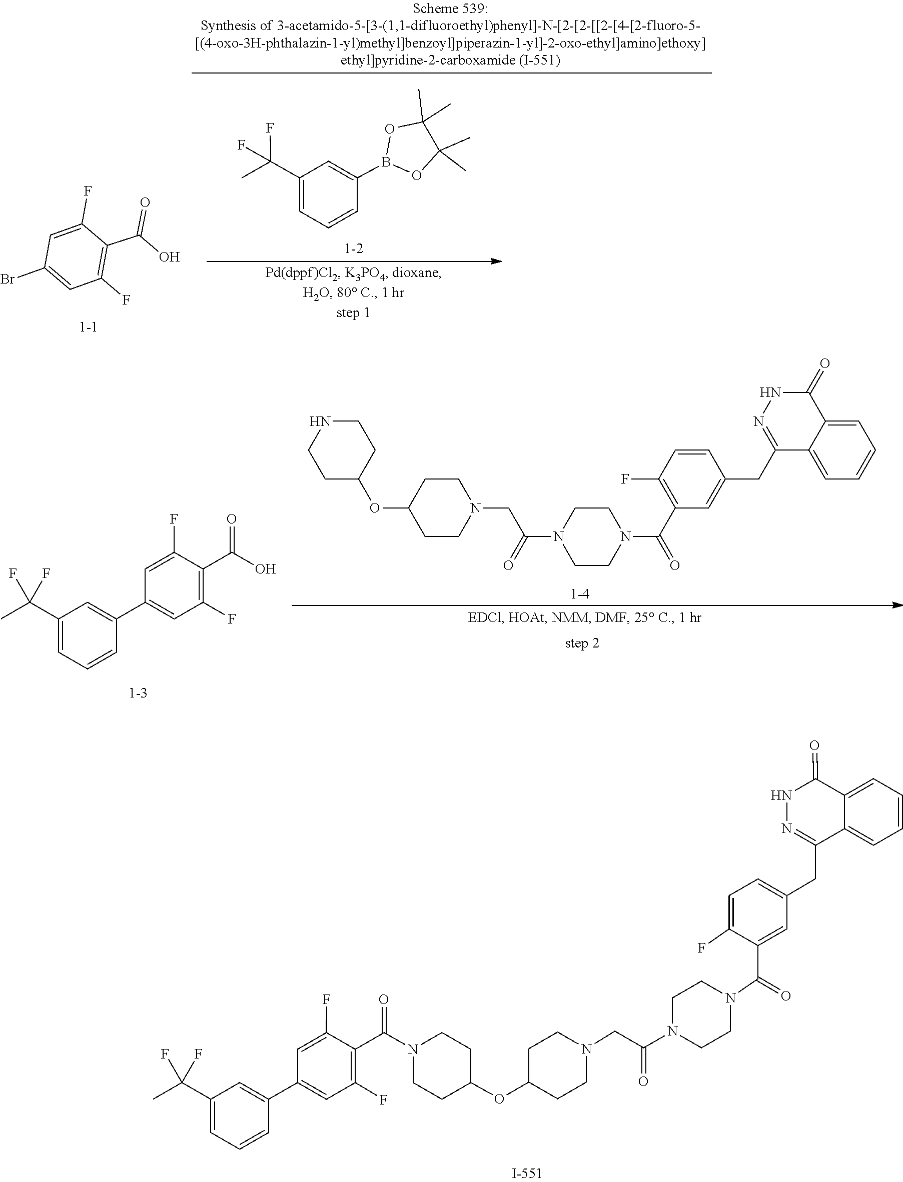 [SMILES: O=C(O)c1c(F)cc(Br)cc1F ; CC(F)(F)c1cccc(B2OC(C)(C)C(C)(C)O2)c1 ; CC(F)(F)c1cccc(-c2cc(F)c(C(=O)O)c(F)c2)c1 ; O=C(CN1CCC(OC2CCNCC2)CC1)N1CCN(C(=O)c2cc(Cc3n[nH]c(=O)c4ccccc34)ccc2F)CC1 ; CC(F)(F)c1cccc(-c2cc(F)c(C(=O)N3CCC(OC4CCN(CC(=O)N5CCN(C(=O)c6cc(Cc7n[nH]c(=O)c8ccccc78)ccc6F)CC5)CC4)CC3)c(F)c2)c1]

### Step 1: Synthesis of Intermediate 1-3

A mixture of Intermediate 1-1 (1 g, 4.22 mmol, 1 eq), Intermediate 1-2 (1.36 g, 5.06 mmol, 1.2 eq), K₃PO₄ (2.69 g, 12.66 mmol, 3 eq) and Pd(dppf)Cl₂ (308.74 mg, 421.94 μmol, 0.1 eq) in dioxane (10 mL) and H₂O (2 mL) was degassed and purged with N₂ 3 times, and then the mixture was stirred at 80° C. for 1 hr under N₂ atmosphere. The residue was diluted with H₂O (20 mL) and extracted with EA (20 mL*4), dried over Na₂SO₄, filtered and concentrated under reduced pressure to give a residue. The water phase was lyophilized to give a residue. To the crude product was added MeOH and then filtered and concentrated under reduced pressure to give a residue. The crude product was purified by reversed-phase chromatography (0.1% NH₃·H₂O), which was concentrated and lyophilized to afford Intermediate 1-3 (740 mg, 2.48 mmol, 58.81% yield) as a brown solid. LCMS (Method F): Rt=0.312 min, (M−H)=296.9.

### Step 2: Synthesis of I-551

To a solution of Intermediate 1-3 (20 mg, 67.06 μmol, 1 eq) in DMF (0.5 mL) was added HOAt (9.13 mg, 67.06 μmol, 9.38 μL, 1 eq), EDCI (38.57 mg, 201.19 μmol, 3 eq), NMM (33.92 mg, 335.31 μmol, 36.86 μL, 5 eq) and Intermediate 1-4 (50.47 mg, 80.47 μmol, 1.2 eq, HCl salt). The mixture was stirred at 25° C. for 1 hr. The residue was diluted with H₂O (5 mL) and extracted with EA (5 mL*3), dried over Na₂SO₄, filtered and concentrated under reduced pressure to give a residue. The combined crude product was purified by prep-HPLC (column: CD02-Waters Xbidge BEH C18 150*25*10 um; mobile phase: [water (NH₃H₂O)-ACN]; gradient: 40%-70% B over 10 min), and the eluent was concentrated and lyophilized to afford I-551 (48.2 mg, 55.34 μmol, 82.53% yield) as a white solid. LCMS (Method D): Rt=0.413 min, (M+H)=871.4. ¹H NMR (400 MHz, METHANOL-d4) δ=8.37 (d, J=7.6 Hz, 1H), 7.97-7.93 (m, 1H), 7.90-7.82 (m, 2H), 7.81 (s, 1H), 7.77 (d, J=6.8 Hz, 1H), 7.63-7.56 (m, 2H), 7.52-7.46 (m, 1H), 7.46-7.41 (m, 2H), 7.37 (s, 1H), 7.16 (t, J=8.8 Hz, 1H), 4.38 (s, 2H), 4.10-4.03 (m, 1H), 3.83-3.71 (m, 4H), 3.67-3.47 (m, 7H), 3.28-3.12 (m, 4H), 2.77 (d, J=2.4 Hz, 2H), 2.32-2.22 (m, 2H), 2.04-1.93 (m, 3H), 1.87 (s, 2H), 1.68-1.55 (m, 4H), 1.28 (s, 2H). ¹⁹F NMR (376 MHz, METHANOL-d₄), δ=−88.799, −114.934, −120.777.

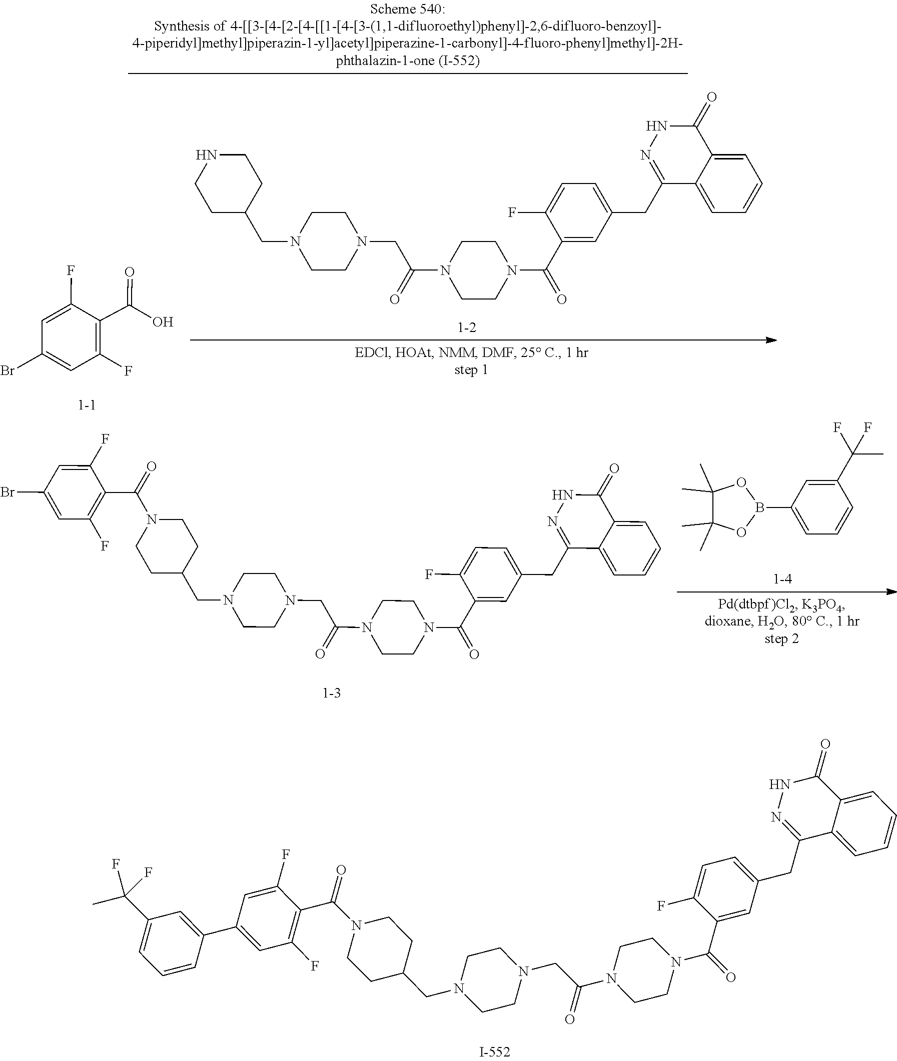

### Step 1: Synthesis of Intermediate 1-3

To a solution of Intermediate 1-1 (100 mg, 421.94 μmol, 1 eq) in DMF (1 mL) was added HOAt (57.43 mg, 421.94 μmol, 59.02 μL, 1 eq), EDCI (242.66 mg, 1.27 mmol, 3 eq), NMM (213.39 mg, 2.11 mmol, 231.95 μL, 5 eq) and Intermediate 1-2 (317.05 mg, 506.33 μmol, 1.2 eq, HCl salt). The mixture was stirred at 25° C. for 1 hr. The residue was diluted with H₂O (5 mL) and extracted with EA (5 mL*3), dried over Na₂SO₄, filtered and concentrated under reduced pressure to give a residue. The residue was purified by flash silica gel chromatography (ISCO; 12 g SepaFlash Silica Flash Column, Eluent of 0˜100% Methanol/Dichloromethane @40 mL/min). The eluent was concentrated under reduced pressure to give Intermediate 1-3 (300 mg, 370.97 μmol, 87.92% yield) as a yellow solid. LCMS (Method D): Rt=0.328 min, [M+H]⁺=808.2.

### Step 2: Synthesis of I-552

A mixture of Intermediate 1-4 (30 mg, 111.90 μmol, 1.2 eq), Intermediate 1-3 (75.41 mg, 93.25 μmol, 1 eq), K₃PO₄ (59.38 mg, 279.74 μmol, 3 eq) and Pd(dtbpf)Cl₂ (6.08 mg, 9.32 μmol, 0.1 eq) in dioxane (1 mL) and H₂O (0.2 mL) was degassed and purged with N₂ 3 times, and then the mixture was stirred at 80° C. for 1 hr under N₂ atmosphere. The residue was diluted with H₂O (10 mL) and extracted with EA (10 mL*3), dried over Na₂SO₄, filtered and concentrated under reduced pressure to give a residue. The residue was purified by prep-HPLC (column: CD01-Phenomenex luna C18 150*25*10 um; mobile phase: [water (FA)-ACN]; gradient: 20%-500% B over 11 min), which was concentrated and lyophilized to afford I-552 (34.65 mg, 38.27 μmol, 41.04% yield, 96.080% purity) as a yellow solid. LCMS (Method D): Rt=0.387 min, [M+H]⁺=870.4. ¹H NMR (400 MHz, METHANOL-d₄) δ=8.40-8.33 (m, 1H), 7.99-7.92 (m, 1H), 7.90-7.83 (m, 2H), 7.81 (s, 1H), 7.78 (d, J=6.8 Hz, 1H), 7.64-7.56 (m, 2H), 7.50 (s, 1H), 7.45-7.42 (m, 2H), 7.40-7.34 (m, 1H), 7.17 (t, J=8.8 Hz, 1H), 4.71 (d, J=11.6 Hz, 1H), 4.39 (s, 2H), 3.83-3.72 (m, 2H), 3.71-3.60 (m, 3H), 3.55-3.42 (m, 4H), 3.37 (s, 2H), 3.25-3.16 (m, 1H), 2.93 (t, J=12.0 Hz, 1H), 2.83-2.64 (m, 8H), 2.49-2.45 (m, 2H), 1.98 (t, J=18.4 Hz, 5H), 1.86-1.79 (m, 1H), 1.31-1.17 (m, 2H). ¹⁹F NMR (377 MHz, METHANOL-d₄), δ=−88.837, −114.901, −120.807.

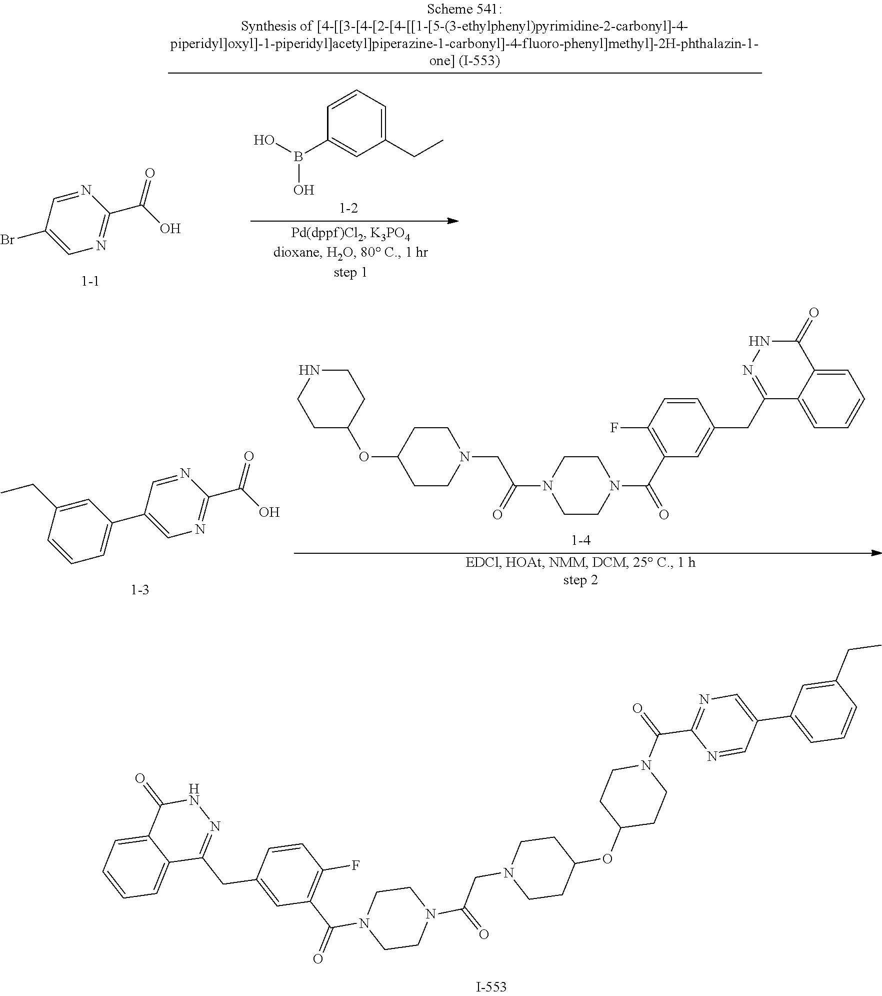 [SMILES: O=C(O)c1ncc(Br)cn1 ; CCc1cccc(B(O)O)c1 ; CCc1cccc(-c2cnc(C(=O)O)nc2)c1 ; O=C(CN1CCC(OC2CCNCC2)CC1)N1CCN(C(=O)c2cc(Cc3n[nH]c(=O)c4ccccc34)ccc2F)CC1 ; "EDCl, HOAt, NMM, DCM, 25° C., 1 hstep 2" ; CCc1cccc(-c2cnc(C(=O)N3CCC(OC4CCN(CC(=O)N5CCN(C(=O)c6cc(Cc7n[nH]c(=O)c8ccccc78)ccc6F)CC5)CC4)CC3)nc2)c1]

### Step 1: Synthesis of Intermediate 1-3

A mixture of intermediate 1-1 (200 mg, 985.25 μmol, 1 eq), intermediate 1-2 (221.66 mg, 1.48 mmol, 1.5 eq), K₃PO₄ (418.27 mg, 1.97 mmol, 2 eq), cyclopentyl (diphenyl) phosphane; dichloropalladium;iron (72.09 mg, 98.53 μmol, 0.1 eq) in dioxane (2 mL) and H₂O (0.4 mL) was degassed and purged with N₂ 3 times, and then the mixture was stirred at 80° C. for 1 hr under N₂ atmosphere. The residue was diluted with H₂O (5 mL) and extracted with EA (5 mL*3), the combined organic phase was dried over Na₂SO₄, filtered and concentrated under reduced pressure to give a residue. The crude product was purified by reversed-phase chromatography (0.1% FA condition). The eluent was concentrated under reduced pressure to remove ACN, then lyophilized to give Intermediate 1-3 (80 mg, 350.50 μmol, 35.57% yield) as a yellow solid. LCMS (Method D): Rt=0.375 min, [M+H]⁺=229.1.

### Step 2: Synthesis of I-553

To a solution of intermediate 1-3 (70 mg, 306.69 μmol, 1 eq) in DMF (0.7 mL) was added EDCI (293.96 mg, 1.53 mmol, 5 eq), NMM (310.20 mg, 3.07 mmol, 337.18 μL, 10 eq) and HOAt (83.49 mg, 613.37 μmol, 85.80 μL, 2 eq) then the intermediate 1-4 (181.16 mg, 306.69 μmol, 1 eq) was added into the mixture. The mixture was stirred at 25° C. for 1 hr. The residue was diluted with H₂O (5 mL) and extracted with EA (5 mL*3), the combined organic phase was dried over Na₂SO₄, filtered and concentrated under reduced pressure to give a residue. The crude product was purified by reversed-phase chromatography (0.1% FA condition). The eluent was concentrated under reduced pressure to remove ACN, then lyophilized to give I-553 (74 mg, 86.88 μmol, 28.33% yield, 99.436% purity, FA salt) as a white solid. LCMS (Method D): Rt=0.351 min, [M+H]⁺=801.3. ¹H NMR (400 MHz, METHANOL-d₄) δ=9.13 (s, 2H), 8.45 (br s, 1H), 8.37 (br d, J=7.6 Hz, 1H), 7.94 (br d, J=3.6 Hz, 1H), 7.91-7.79 (m, 2H), 7.60 (s, 1H), 7.56 (br d, J=7.6 Hz, 1H), 7.53-7.43 (m, 2H), 7.40-7.33 (m, 2H), 7.17 (t, J=8.8 Hz, 1H), 4.38 (s, 2H), 4.10-3.98 (m, 1H), 3.81 (br d, J=4.0 Hz, 2H), 3.77-3.58 (m, 7H), 3.57-3.50 (m, 2H), 3.48 (br s, 2H), 3.30-3.16 (m, 2H), 3.07 (br d, J=3.2 Hz, 2H), 2.78-2.73 (m, 4H), 1.98 (br s, 3H), 1.92-1.57 (m, 5H), 1.30 (t, J=7.6 Hz, 3H). ¹⁹F NMR (377 MHz, METHANOL-d₄) δ=−120.749.

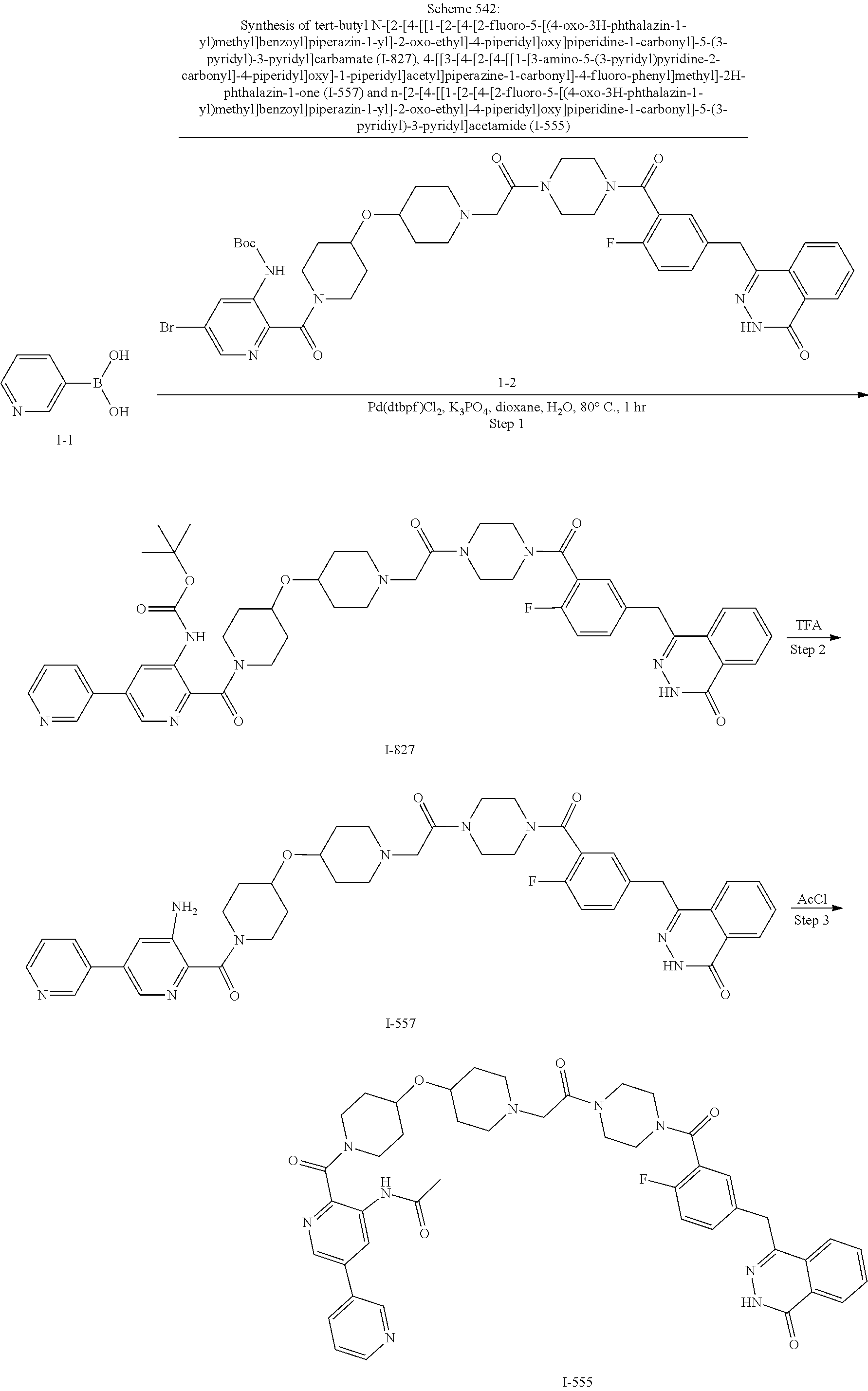

### Step 1: Synthesis of I-827

To a solution of intermediate 1-2 (20 mg, 22.48 μmol, 1 eq) and intermediate 1-1 (5.53 mg, 44.95 μmol, 2 eq) in dioxane (0.5 mL) and H₂O (0.1 mL) was added K₃PO₄ (14.31 mg, 67.43 μmol, 3 eq) and ditert-butyl(cyclopenta-1,4-dien-1-yl)phosphane;dichloropalladium;iron (1.46 mg, 2.25 μmol, 0.1 eq). The mixture was stirred at 80° C. for 1 hr. The mixture was diluted with water (5 mL) and extracted with DCM (5 mL*3). The combined organic layer was dried over Na₂SO₄, filtered and concentrated in vacuo to give a residue. The residue was purified by prep-HPLC (column: CD01-Phenomenex luna C18 150*25*10 um; mobile phase: [water (FA)-ACN]; gradient: 11%-41% B over 11 min). Then the eluent was concentrated in vacuo and lyophilized to give I-827 (8.19 mg, 8.68 μmol, 38.62% yield, 98.990% purity, FA salt) as a white solid. LCMS (Method D): Rt=0.323 min, [M+H]⁺=888.2. ¹H NMR (400 MHz, METHANOL-d₄) δ=8.89 (d, J=1.6 Hz, 1H), 8.65-8.58 (m, 2H), 8.47 (d, J=1.6 Hz, 1H), 8.42-8.33 (m, 2H), 8.19 (d, J=8.0 Hz, 1H), 7.98-7.93 (m, 1H), 7.90-7.82 (m, 2H), 7.60-7.58 (m, 1H), 7.53-7.47 (m, 1H), 7.37 (t, J=6.8 Hz, 1H), 7.17 (t, J=8.8 Hz, 1H), 4.39 (s, 2H), 4.05 (d, J=5.6 Hz, 1H), 3.87 (s, 1H), 3.81 (s, 2H), 3.76 (s, 2H), 3.70 (d, J=4.8 Hz, 2H), 3.65-3.48 (m, 4H), 3.45 (s, 1H), 3.35 (d, J=3.6 Hz, 3H), 3.19-3.10 (m, 2H), 2.97-2.79 (m, 2H), 2.00 (d, J=3.2 Hz, 3H), 1.94-1.78 (m, 3H), 1.69 (s, 2H), 1.53 (s, 9H). ¹⁹F NMR (376 MHz, METHANOL-d₄) δ=−120.757.

### Step 2: Synthesis of I-557

To a solution of I-827 (20 mg, 22.52 μmol, 1 eq) in DCM (0.5 mL) was added TFA (153.50 mg, 1.35 mmol, 0.1 mL, 59.77 eq). The mixture was stirred at 25° C. for 0.5 hr. The reaction mixture was concentrated in vacuo to give a residue. The residue was lyophilized directly without further purification to give the product. I-557 (9.58 mg, 10.32 μmol, 45.80% yield, 97.121% purity, TFA salt) was obtained as a yellow solid. LCMS (Method D): Rt=0.272 min, [M+H]⁺=788.5. ¹H NMR (400 MHz, METHANOL-d₄) δ =9.14 (s, 1H), 8.85 (d, J=5.6 Hz, 1H), 8.75 (d, J=8.0 Hz, 1H), 8.37-8.35 (m, 1H), 8.24 (s, 1H), 8.07-8.05 (m, 1H), 7.97-7.93 (m, 1H), 7.89-7.81 (m, 2H), 7.61 (d, J=1.6 Hz, 1H), 7.51-7.50 (m, 1H), 7.39-7.34 (m, 1H), 7.17 (t, J=8.8 Hz, 1H), 4.39 (s, 2H), 4.31 (s, 1H), 4.24 (s, 1H), 4.12-3.99 (m, 1H), 3.96 (s, 1H), 3.86-3.75 (m, 4H), 3.72 (s, 1H), 3.65 (s, 2H), 3.55 (d, J=4.8 Hz, 1H), 3.53-3.47 (m, 2H), 3.45 (s, 1H), 3.36 (s, 4H), 3.20-3.05 (m, 1H), 2.28-2.15 (m, 1H), 2.06 (s, 3H), 1.95-1.81 (m, 2H), 1.74-1.61 (m, 2H). ¹⁹F NMR (376 MHz, METHANOL-d₄) δ=−77.412, −120.412, −120.66.

### Step 3: Synthesis of I-555

To a solution of I-557 (60 mg, 66.53 μmol, 1 eq, TFA salt) and acetyl chloride (5.22 mg, 66.53 μmol, 4.73 μL, 1 eq) in DCM (0.5 mL) was added DIEA (25.79 mg, 199.58 μmol, 34.76 μL, 3 eq). The mixture was stirred at 0° C. for 0.5 hr. The mixture was diluted with water (5 mL) and extracted with DCM (5 mL*3). The combined organic layer was dried over Na₂SO₄, filtered and concentrated in vacuo to give a residue. The residue was purified by prep-HPLC (column: CD07-Daisogel SP-100-8-ODS-PK 150*25*10 um; mobile phase: [water (NH₄HCO₃)-ACN]; gradient: 12%-42% B over 10 min). Then the eluent was concentrated in vacuo and lyophilized to give the product. I-555 (23.86 mg, 28.75 μmol, 43.22% yield, 100% purity) was obtained as a white solid. LCMS (Method D): Rt=0.314 min, [M+H]⁺=830.6. ¹H NMR (400 MHz, METHANOL-d₄) δ=8.90 (d, J=2.0 Hz, 1H), 8.70 (d, J=1.6 Hz, 1H), 8.63-8.62 (m, 1H), 8.39-8.35 (m, 2H), 8.19 (d, J=8.0 Hz, 1H), 7.97-7.93 (m, 1H), 7.89-7.82 (m, 2H), 7.60-7.58 (m, 1H), 7.49 (d, J=4.4 Hz, 1H), 7.38 (s, 1H), 7.16 (t, J=9.2 Hz, 1H), 4.39 (s, 2H), 4.08-4.01 (m, 1H), 3.80 (s, 2H), 3.76-3.71 (m, 2H), 3.66 (s, 2H), 3.56 (d, J=4.0 Hz, 2H), 3.53-3.48 (m, 2H), 3.26 (s, 2H), 3.19 (s, 1H), 2.81-2.73 (m, 2H), 2.32-2.22 (m, 2H), 2.17 (s, 3H), 2.01-1.82 (m, 5H), 1.74-1.52 (m, 5H). ¹⁹F NMR (376 MHz, METHANOL-d₄) δ=−120.757.

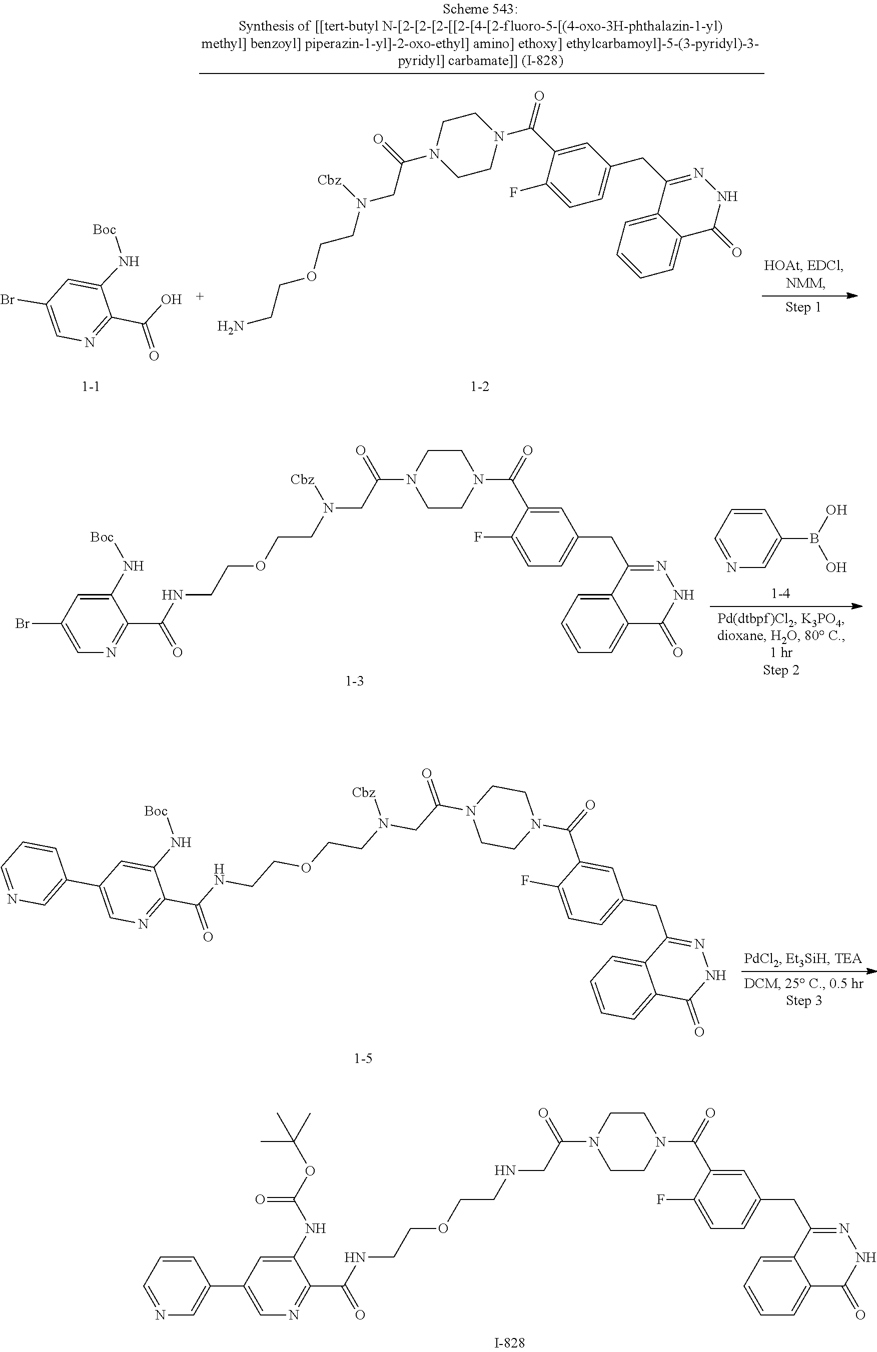 [SMILES: CC(C)(C)OC(=O)Nc1cc(Br)cnc1C(=O)O ; NCCOCCN(CC(=O)N1CCN(C(=O)c2cc(Cc3n[nH]c(=O)c4ccccc34)ccc2F)CC1)C(=O)OCc1ccccc1 ; CC(C)(C)OC(=O)Nc1cc(Br)cnc1C(=O)NCCOCCN(CC(=O)N1CCN(C(=O)c2cc(Cc3n[nH]c(=O)c4ccccc34)ccc2F)CC1)C(=O)OCc1ccccc1 ; OB(O)c1cccnc1 ; CC(C)(C)OC(=O)Nc1cc(-c2cccnc2)cnc1C(=O)NCCOCCN(CC(=O)N1CCN(C(=O)c2cc(Cc3n[nH]c(=O)c4ccccc34)ccc2F)CC1)C(=O)OCc1ccccc1 ; CC(C)(C)OC(=O)Nc1cc(-c2cccnc2)cnc1C(=O)NCCOCCNCC(=O)N1CCN(C(=O)c2cc(Cc3n[nH]c(=O)c4ccccc34)ccc2F)CC1]

### Step 1: Synthesis of Intermediate 1-3

To a solution of intermediate 1-1 (931 mg, 2.94 mmol, 1 eq) and intermediate 1-2 (2 g, 2.94 mmol, 1 eq, HCl salt) in DMF (20 mL) was added EDCI (1.69 g, 8.81 mmol, 3 eq), HOAt (399.65 mg, 2.94 mmol, 410.74 μL, 1 eq) and NMM (1.48 g, 14.68 mmol, 1.61 mL, 5 eq). The mixture was stirred at 25° C. for 10 hr. The reaction mixture was poured into H₂O (20 mL) and extracted with EA (20 mL*3). The combined organic layers were dried over Na₂SO₄, filtered and concentrated under reduced pressure to give a residue. The residue was purified by flash silica gel chromatography (ISCO; 40 g SepaFlashSilica Flash Column, Eluent of 0˜100% DCM/MeOH@36 mL/min, DCM/MeOH=10:1, Rf=0.5) and the eluent was concentrated under reduced pressure to give Intermediate 1-3 (600 mg, crude) as a yellow gum. LCMS (Method D): Rt=0.492 min, [M+H]⁺=943.1.

### Step 2: Synthesis of Intermediate 1-5

To a solution of intermediate 1-3 (200 mg, 211.91 μmol, 1 eq) and intermediate 1-4 (31.26 mg, 254.29 μmol, 1.2 eq) in dioxane (2 mL) and H₂O (0.4 mL) was added ditert-butyl (cyclopenta-1,4-dien-1-yl) phosphane; dichloropalladium; iron (13.81 mg, 21.19 μmol, 0.1 eq) and K₃PO₄ (89.96 mg, 423.81 μmol, 2 eq). The mixture was stirred at 80° C. for 1 hour under N₂. The reaction mixture was concentrated under reduced pressure to give a residue. The residue was purified by flash silica gel chromatography (ISCO; 20 g SepaFlashSilica Flash Column, Eluent of 0˜100% DCM/MeOH@36 mL/min, DCM/MeOH=10:1, Rf=0.5) and the eluent was concentrated under reduced pressure to give Intermediate 1-5 (200 mg, crude) as a brown solid. LCMS (Method D): Rt=0.418 min, [M+H]⁺=942.3. ¹H NMR (400 MHz, METHANOL-d₄) δ=9.14-8.94 (m, 1H), 8.93-8.80 (m, 1H), 8ss.63 (d, J=3.2 Hz, 1H), 8.57-8.41 (m, 1H), 8.39-8.29 (m, 1H), 8.22-8.07 (m, 1H), 7.96-7.91 (m, 1H), 7.89-7.79 (m, 2H), 7.64-7.55 (m, 1H), 7.51-7.41 (m, 1H), 7.34 (s, 2H), 5.10 (d, J=17.6 Hz, 2H), 5.00 (br s, 1H), 4.94-4.89 (m, 1H), 4.79 (d, J=1.6 Hz, 1H), 4.36 (d, J=9.2 Hz, 2H), 4.30-4.16 (m, 2H), 3.77-3.71 (m, 1H), 3.63 (d, J=9.2 Hz, 4H), 3.61-3.52 (m, 7H), 3.52-3.46 (m, 3H), 3.44-3.37 (m, 2H), 3.35 (s, 2H), 3.25 (s, 2H), 3.17-3.04 (m, 1H), 2.99 (s, 1H), 2.86 (s, 1H), 1.92 (s, 1H), 1.65-1.55 (m, 2H).

### Step 3: Synthesis of I-828

To a solution of intermediate 1-5 (50 mg, 53.08 μmol, 1 eq) in DCM (0.5 mL) was added dichloropalladium (4.71 mg, 26.54 μmol, 0.5 eq), triethylsilane (74.06 mg, 636.94 μmol, 101.73 μL, 12 eq) and TEA (16.11 mg, 159.24 μmol, 22.16 μL, 3 eq). The mixture was stirred at 25° C. for 0.5 hr. The reaction mixture was concentrated under reduced pressure to give a residue. The residue was purified by prep-HPLC (column: CD04-Welch Utimate C18 150*25*7 um; mobile phase: [water (FA)-ACN]; gradient: 5%-35% B over 8 min) and the eluent was concentrated to remove MeCN and then lyophilized to give I-828 (14.25 mg, 16.69 μmol, 31.44% yield, 100% purity, FA salt) as a white solid. LCMS (Method D): Rt=0.337 min, [M+H]⁺=808.3. ¹H NMR (400 MHz, METHANOL-d₄) δ=9.08 (d, J=2.4 Hz, 1H), 8.89 (d, J=2.0 Hz, 1H), 8.70-8.59 (m, 1H), 8.54 (d, J=7.2 Hz, 1H), 8.36 (d, J=7.6 Hz, 1H), 8.19 (d, J=8.0 Hz, 1H), 8.00-7.92 (m, 1H), 7.90-7.80 (m, 2H), 7.67-7.54 (m, 1H), 7.49 (d, J=2.8 Hz, 1H), 7.35 (t, J=6.4 Hz, 1H), 7.18-7.12 (m, 1H), 4.84 (s, 1H), 4.38 (d, J=4.0 Hz, 2H), 3.93 (s, 1H), 3.84 (s, 1H), 3.75-3.71 (m, 6H), 3.68-3.62 (m, 3H), 3.50 (s, 2H), 3.29 (s, 2H), 3.14-3.08 (m, 2H), 1.55 (s, 9H). ¹⁹F NMR (376 MHz, METHANOL-d₄) δ=−120.674.

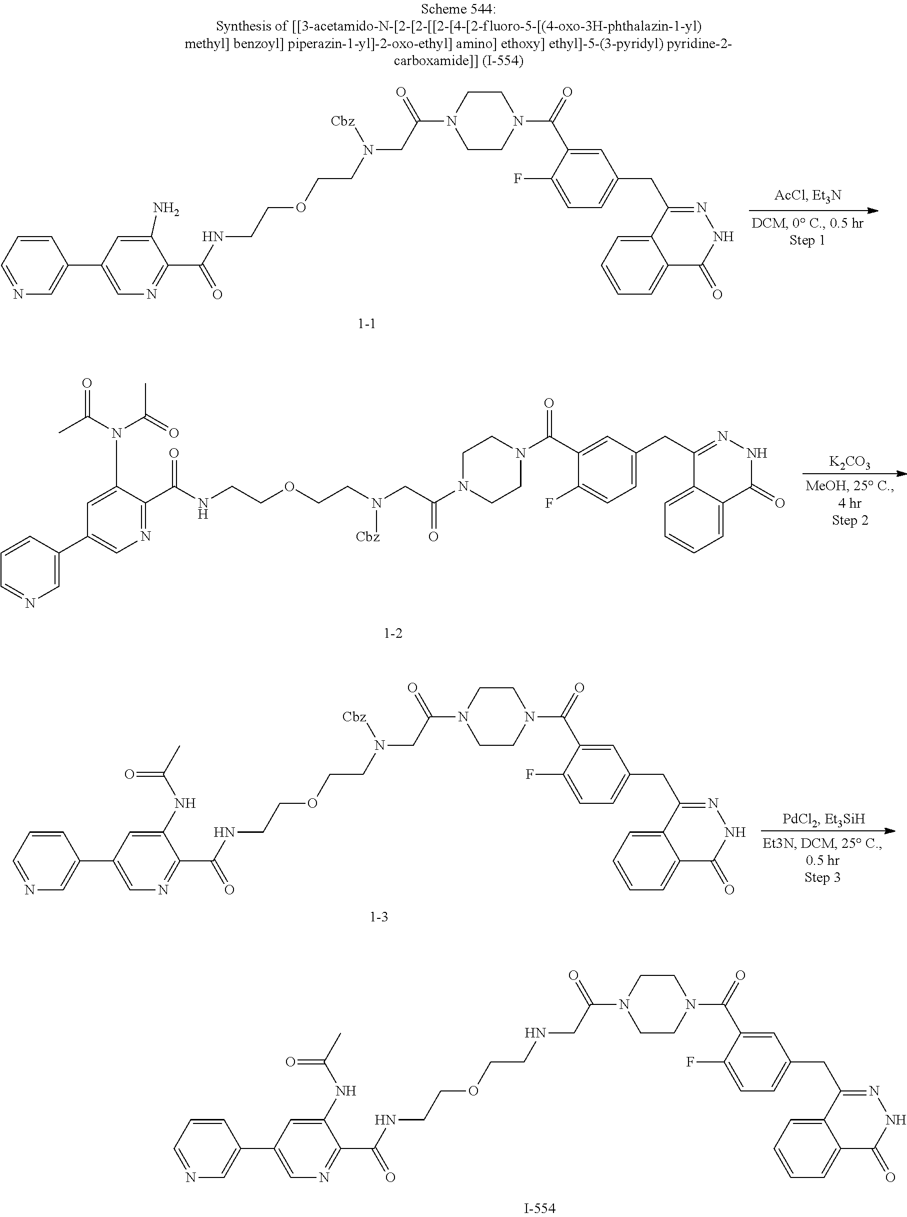

### Step 1: Synthesis of Intermediate 1-2

To a solution of intermediate 1-1 (85 mg, 88.92 μmol, 1 eq, TFA salt) in DCM (0.9 mL) was added Et₃N (18.00 mg, 177.84 μmol, 24.75 μL, 2 eq) and acetyl chloride (6.98 mg, 88.92 μmol, 6.32 μL, 1 eq). The mixture was stirred at 0° C. for 0.5 hr. The reaction mixture was concentrated under reduced pressure to give a product. The product was used for the next step without purification to give Intermediate 1-2 (80 mg, 86.40 μmol, 97.16% yield) as a yellow gum. LCMS (Method D): Rt=0.379 min, [M+H]⁺=926.2.

### Step 2: Synthesis of Intermediate 1-3

To a solution of intermediate 1-2 (80 mg, 86.40 μmol, 1 eq) in MeOH (0.8 mL) was added K₂CO₃ (23.88 mg, 172.79 μmol, 2 eq). The mixture was stirred at 25° C. for 4 hr. The reaction mixture was concentrated under reduced pressure to give a residue. The residue was purified by reversed-phase chromatography (FA condition) and the eluent was concentrated to remove MeCN and then lyophilized to give Intermediate 1-3 (50 mg, 56.57 μmol, 65.47% yield) as a yellow solid. LCMS (Method D): Rt=0.363 min, [M+H]⁺=884.3.

### Step 3: Synthesis of I-554

To a solution of intermediate 1-3 (40 mg, 45.25 μmol, 1 eq) in DCM (0.4 mL) was added PdCl₂ (4.01 mg, 22.63 μmol, 0.5 eq), Et₃N (13.74 mg, 135.76 μmol, 18.90 μL, 3 eq) and Et₃SiH (63.14 mg, 543.04 μmol, 86.73 μL, 12 eq). The mixture was stirred at 25° C. for 0.5 hr. The reaction mixture was concentrated under reduced pressure to give a residue. The residue was purified by prep-HPLC (column: CD01-Phenomenex luna C18 150*25*10 um; mobile phase: [water (FA)-ACN]; gradient: 3%-33% B over 8 min) and the eluent was concentrated to remove MeCN and then lyophilized to give I-554 (7 mg, 8.61 μmol, 19.02% yield, 97.838% purity, FA salt) as a white solid. LCMS (Method D): Rt=0.276 min, [M+H]⁺=750.3. ¹H NMR (400 MHz, METHANOL-d₄) δ=9.29 (s, 1H), 8.87 (s, 1H), 8.61 (t, J=7.2 Hz, 2H), 8.44 (s, 1H), 8.35 (d, J=7.6 Hz, 1H), 8.15 (d, J=7.6 Hz, 1H), 7.96-7.90 (m, 1H), 7.89-7.79 (m, 2H), 7.63-7.54 (m, 1H), 7.52-7.45 (m, 1H), 7.34 (t, J=6.0 Hz, 1H), 7.14 (t, J=8.8 Hz, 1H), 4.78-4.59 (m, 1H), 4.37 (s, 2H), 4.13-3.96 (m, 2H), 3.81 (s, 2H), 3.74 (s, 4H), 3.67 (s, 3H), 3.48 (d, J=2.0 Hz, 2H), 3.25 (d, J=1.6 Hz, 4H), 2.25 (s, 3H). ¹⁹F NMR (376 MHz, METHANOL-d₄) δ=−120.665.

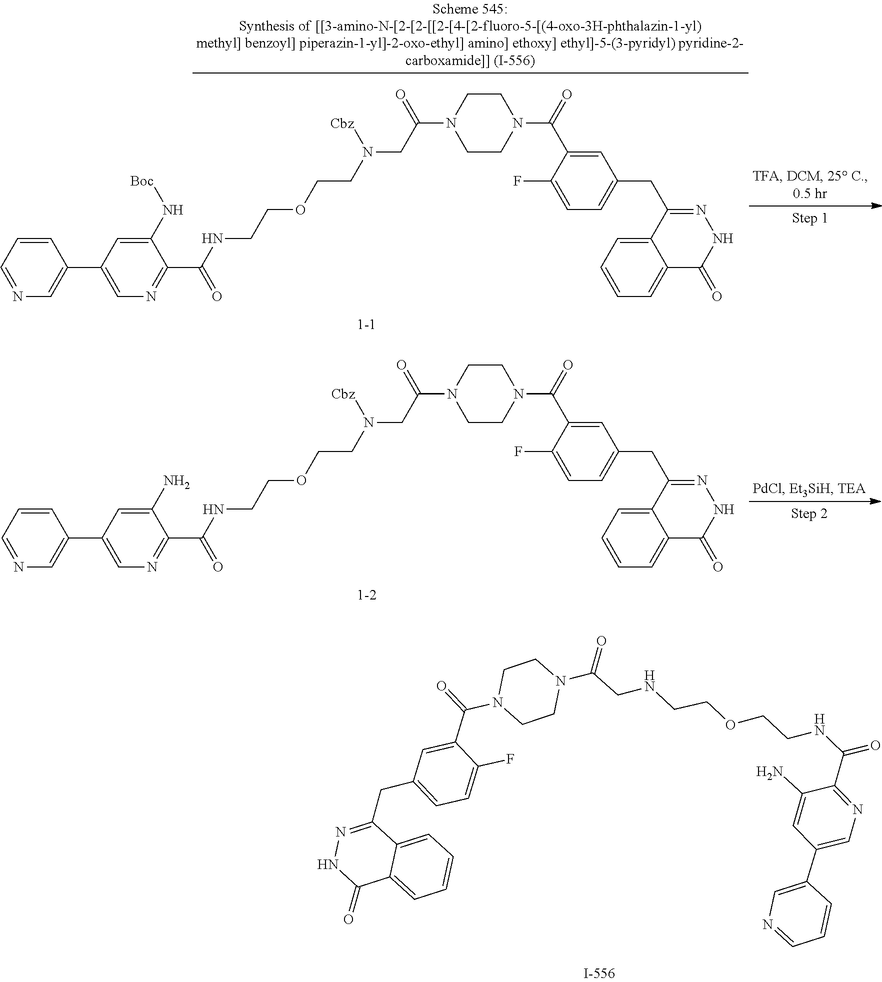 [SMILES: CC(C)(C)OC(=O)Nc1cc(-c2cccnc2)cnc1C(=O)NCCOCCN(CC(=O)N1CCN(C(=O)c2cc(Cc3n[nH]c(=O)c4ccccc34)ccc2F)CC1)C(=O)OCc1ccccc1 ; Nc1cc(-c2cccnc2)cnc1C(=O)NCCOCCN(CC(=O)N1CCN(C(=O)c2cc(Cc3n[nH]c(=O)c4ccccc34)ccc2F)CC1)C(=O)OCc1ccccc1 ; Nc1cc(-c2cccnc2)cnc1C(=O)NCCOCCNCC(=O)N1CCN(C(=O)c2cc(Cc3n[nH]c(=O)c4ccccc34)ccc2F)CC1]

### Step 1: Synthesis of Intermediate 1-2

To a solution of Intermediate 1-1 (50 mg, 53.08 μmol, 1 eq) in DCM (0.5 mL) was added TFA (153.50 mg, 1.35 mmol, 100.00 μL, 25.36 eq). The mixture was stirred at 25° C. for 1 hr. The reaction mixture was concentrated under reduced pressure to give Intermediate 1-2 (45 mg, crude, TFA salt) as a yellow gum. LCMS (Method D): Rt=0.345 min, [M+H]⁺=842.3.

### Step 2: Synthesis of I-556

To a solution of Intermediate 1-2 (45 mg, 47.08 μmol, 1 eq, TFA salt) in DCM (0.5 mL) was added PdCl₂ (4.17 mg, 23.54 μmol, 0.5 eq), Et₃SiH (65.69 mg, 564.91 μmol, 90.23 μL, 12 eq) and Et₃N (14.29 mg, 141.23 μmol, 19.66 μL, 3 eq). The mixture was stirred at 25° C. for 1 hr. The reaction mixture was concentrated under reduced pressure to give a residue. The residue was purified by prep-HPLC (column: CD03-Welch Xtimate C18 150*25*5 um; mobile phase: [water (FA)-ACN]; gradient: 4%-34% B over 10 min) and the eluent was concentrated to remove MeCN and then lyophilized to give I-556 (11.91 mg, 15.63 μmol, 33.20% yield, 98.913% purity, FA salt) as a white solid. LCMS (Method D): Rt=0.261 min, [M+H]⁺=708.2. ¹H NMR (400 MHz, METHANOL-d₄) δ=8.81 (s, 1H), 8.59 (s, 1H), 8.51-8.40 (m, 1H), 8.36 (d, J=7.6 Hz, 1H), 8.10 (d, J=12.4 Hz, 2H), 7.97-7.90 (m, 1H), 7.90-7.78 (m, 2H), 7.59-7.46 (m, 2H), 7.41 (d, J=9.2 Hz, 1H), 7.34 (t, J=6.8 Hz, 1H), 7.15 (t, J=8.8 Hz, 1H), 4.37 (s, 2H), 4.18-3.98 (m, 2H), 3.81 (s, 2H), 3.72 (s, 5H), 3.62 (d, J=4.4 Hz, 2H), 3.54-3.45 (m, 2H), 3.37-3.32 (m, 2H), 3.27 (s, 3H). ¹⁹F NMR (376 MHz, METHANOL-d₄) δ=−120.650.

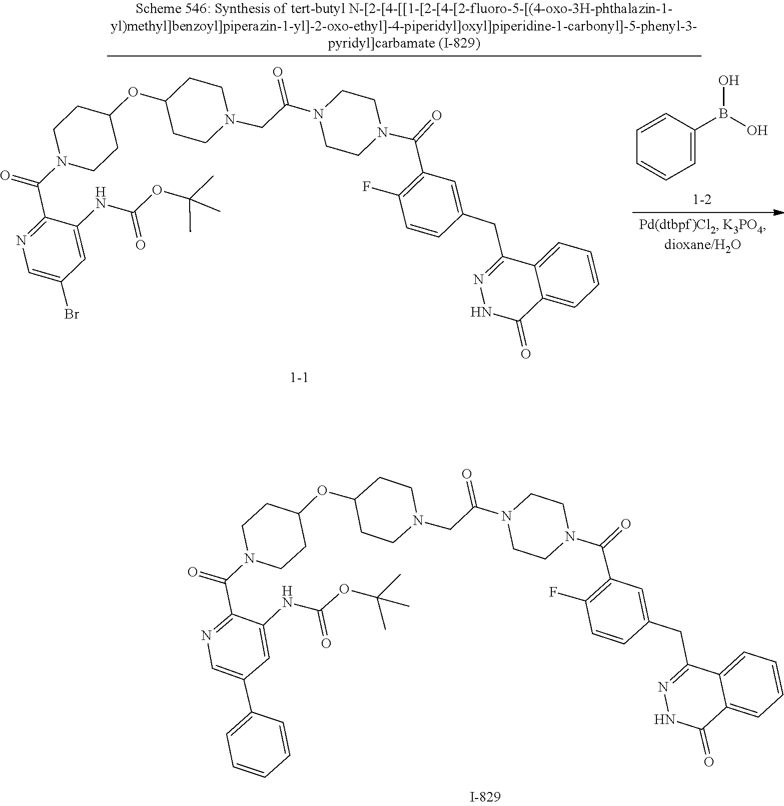 [SMILES: CC(C)(C)OC(=O)Nc1cc(Br)cnc1C(=O)N1CCC(OC2CCN(CC(=O)N3CCN(C(=O)c4cc(Cc5n[nH]c(=O)c6ccccc56)ccc4F)CC3)CC2)CC1 ; OB(O)c1ccccc1 ; CC(C)(C)OC(=O)Nc1cc(-c2ccccc2)cnc1C(=O)N1CCC(OC2CCN(CC(=O)N3CCN(C(=O)c4cc(Cc5n[nH]c(=O)c6ccccc56)ccc4F)CC3)CC2)CC1]

To a solution of intermediate 1-1 (50 mg, 56.19 μmol, 1 eq) and intermediate 1-2 (17.13 mg, 140.48 μmol, 2.5 eq) in dioxane (1 mL) and H₂O (0.2 mL) was added K₃PO₄ (35.78 mg, 168.58 μmol, 3 eq) and ditert-butyl (cyclopenta-1,4-dien-1-yl)phosphane; dichloropalladium;iron (3.66 mg, 5.62 μmol, 0.1 eq), then the mixture was stirred at 80° C. for 1 hour under N₂. The reaction mixture was diluted with water (3 mL) and extracted with EA (5 mL*2). The combined organic layers were concentrated under reduced pressure to give a residue. The crude product was purified by reversed-phase chromatography (0.1% FA condition). The eluent was concentrated under reduced pressure to remove ACN and then lyophilized to give I-829 (24.41 mg, 27.52 μmol, 48.97% yield, 100% purity) as a yellow solid. LCMS (Method D): Rt: 0.410 min, [M+H]⁺=887.6. ¹H NMR (400 MHz, METHANOL-d₄) δ=8.56-8.51 (m, 1H), 8.42 (s, 1H), 8.36 (d, J=7.6 Hz, 1H), 7.94 (d, J=5.6 Hz, 1H), 7.89-7.80 (m, 2H), 7.69 (d, J=7.6 Hz, 2H), 7.53-7.42 (m, 4H), 7.37 (s, 1H), 7.19-7.16 (m, 1H), 4.68-4.49 (m, 1H), 4.38 (s, 2H), 4.06 (s, 1H), 3.85-3.49 (m, 12H), 3.40 (s, 1H), 2.86 (s, 2H), 2.49-2.34 (m, 2H), 1.93 (d, J=9.2 Hz, 4H), 1.67 (s, 5H), 1.53 (s, 9H). ¹⁹F NMR (376 MHz, METHANOL-d₄) δ=−120.755.

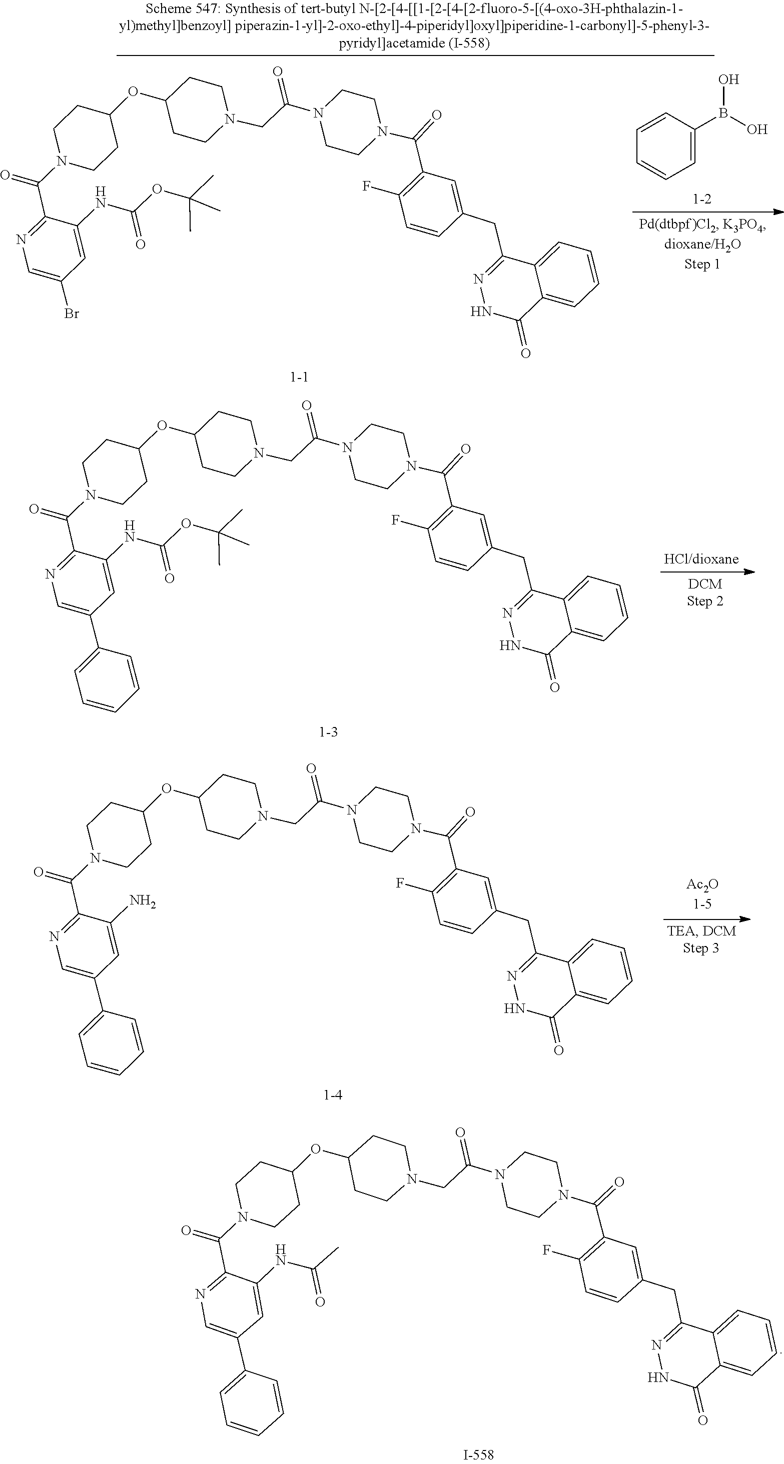 [SMILES: CC(C)(C)OC(=O)Nc1cc(Br)cnc1C(=O)N1CCC(OC2CCN(CC(=O)N3CCN(C(=O)c4cc(Cc5n[nH]c(=O)c6ccccc56)ccc4F)CC3)CC2)CC1 ; OB(O)c1ccccc1 ; CC(C)(C)OC(=O)Nc1cc(-c2ccccc2)cnc1C(=O)N1CCC(OC2CCN(CC(=O)N3CCN(C(=O)c4cc(Cc5n[nH]c(=O)c6ccccc56)ccc4F)CC3)CC2)CC1 ; Nc1cc(-c2ccccc2)cnc1C(=O)N1CCC(OC2CCN(CC(=O)N3CCN(C(=O)c4cc(Cc5n[nH]c(=O)c6ccccc56)ccc4F)CC3)CC2)CC1 ; CC(=O)Nc1cc(-c2ccccc2)cnc1C(=O)N1CCC(OC2CCN(CC(=O)N3CCN(C(=O)c4cc(Cc5n[nH]c(=O)c6ccccc56)ccc4F)CC3)CC2)CC1]

### Step 1: Synthesis of Intermediate 1-3

To a solution of Intermediate 1-1 (100 mg, 112.38 μmol, 1 eq) and Intermediate 1-2 (34.26 mg, 280.96 μmol, 2.5 eq) in dioxane (1 mL) and H₂O (0.2 mL) was added K₃PO₄ (71.57 mg, 337.15 μmol, 3 eq) and ditert-butyl(cyclopenta-1,4-dien-1-yl) phosphane; dichloropalladium; iron (7.32 mg, 11.24 mol, 0.1 eq), then the mixture was stirred at 80° C. for 1 hour. The reaction mixture was diluted with water (80 mL) and extracted with EA (30 mL*2). The combined organic layers were concentrated under reduced pressure to give a residue. Intermediate 1-3 (80 mg, 90.19 μmol, 80.25% yield) was obtained as a yellow solid.

### Step 2: Synthesis of Intermediate 1-4

To a solution of Intermediate 1-3 (80 mg, 90.19 μmol, 1 eq) in DCM (1 mL) was added HCl/dioxane (2 M, 225.48 μL, 5 eq), then the mixture was stirred at 20° C. for 1 hour. The reaction mixture was concentrated under reduced pressure to give a residue. Intermediate 1-4 (70 mg, 85.02 μmol, 94.26% yield, HCl salt) as a yellow solid. LCMS (Method D): Rt=0.334 min, [M+H]⁺=787.2.

### Step 3: Synthesis of I-558

To a solution of Intermediate 1-4 (70 mg, 85.02 μmol, 1 eq, HCl salt) in DCM (1 mL) was added TEA (25.81 mg, 255.05 μmol, 35.50 μL, 3 eq) and Intermediate 1-5 (6.67 mg, 85.02 μmol, 6.05 μL, 1 eq) at 0° C., then the mixture was stirred at 0° C. for 1 hour. The reaction mixture was diluted with water (80 mL) and extracted with DCM (30 mL*2). The combined organic layers were concentrated under reduced pressure to give a residue. The crude product was purified by prep-HPLC (column: CD02-Waters Xbidge BEH C18 150*25*10 um; mobile phase: [water (NH₄HCO₃)-ACN]; gradient: 24%-44% B over 10 min). The eluent was concentrated under reduced pressure to remove MeCN and then lyophilized to give the product. I-558 (14.57 mg, 17.58 μmol, 20.67% yield, 100% purity) was obtained as a white solid. LCMS (Method D): Rt=0.345 min, [M+H]⁺=829.5. ¹H NMR (400 MHz, METHANOL-d4) δ=8.63 (s, 1H), 8.36 (d, J=7.6 Hz, 1H), 8.28 (s, 1H), 7.98-7.91 (m, 1H), 7.89-7.80 (m, 2H), 7.68 (d, J=7.6 Hz, 2H), 7.53-7.42 (m, 4H), 7.37 (s, 1H), 7.18-7.15 (m, 1H), 4.38 (s, 2H), 4.07-3.98 (m, 1H), 3.83-3.48 (m, 11H), 3.38-3.32 (m, 1H), 3.25 (s, 2H), 3.19 (s, 1H), 2.77 (s, 2H), 2.26 (d, J=9.6 Hz, 2H), 2.17 (s, 3H), 1.97-1.82 (m, 4H), 1.71-1.54 (m, 4H). ¹⁹F NMR (400 MHz, METHANOL-d4) δ=−120.742.

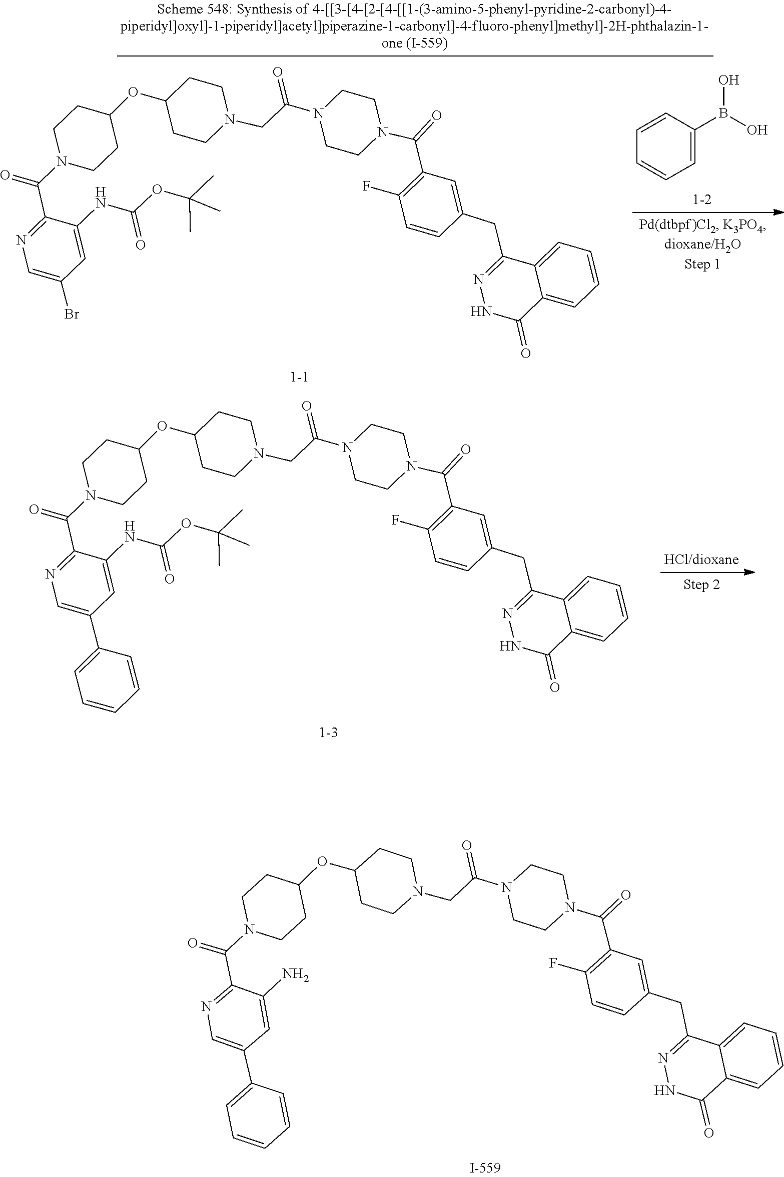 [SMILES: CC(C)(C)OC(=O)Nc1cc(Br)cnc1C(=O)N1CCC(OC2CCN(CC(=O)N3CCN(C(=O)c4cc(Cc5n[nH]c(=O)c6ccccc56)ccc4F)CC3)CC2)CC1 ; OB(O)c1ccccc1 ; CC(C)(C)OC(=O)Nc1cc(-c2ccccc2)cnc1C(=O)N1CCC(OC2CCN(CC(=O)N3CCN(C(=O)c4cc(Cc5n[nH]c(=O)c6ccccc56)ccc4F)CC3)CC2)CC1 ; Nc1cc(-c2ccccc2)cnc1C(=O)N1CCC(OC2CCN(CC(=O)N3CCN(C(=O)c4cc(Cc5n[nH]c(=O)c6ccccc56)ccc4F)CC3)CC2)CC1]

### Step 1: Synthesis of Intermediate 1-3

To a mixture of intermediate 1-1 (200 mg, 224.77 μmol, 1 eq) and intermediate 1-2 (68.51 mg, 561.92 μmol, 2.5 eq) in dioxane (2 mL) and H₂O (0.4 mL) was added K₃PO₄ (143.13 mg, 674.30 μmol, 3 eq) and ditert-butyl(cyclopenta-1,4-dien-1-yl) phosphane; dichloropalladium; iron (14.65 mg, 22.48 mol, 0.1 eq), then the mixture was stirred at 80° C. for 1 hour under N₂. The reaction mixture was diluted with water (5 mL) and extracted with EA (5 mL*2). The combined organic layers were concentrated under reduced pressure to give a residue. Intermediate 1-3 (190 mg, 214.20 μmol, 95.30% yield) was obtained as a yellow solid.

### Step 2: Synthesis of I-559

To a mixture of intermediate 1-3 (50 mg, 56.37 μmol, 1 eq) in DCM (1 mL) was added HCl/dioxane (2 M, 140.92 μL, 5 eq), then the mixture was stirred at 20° C. for 1 hour. To the reaction mixture was added a NaHCO₃ solution (about 10 mL) to adjust the pH to 7-8, and then the mixture was extracted with DCM (10 mL*3). The combined organic layers were concentrated under reduced pressure to give a residue. The crude product was purified by reversed-phase chromatography (0.1% FA condition). The eluent was concentrated under reduced pressure to remove ACN and then lyophilized to give the product. I-559 (9.94 mg, 11.93 μmol, 21.17% yield, 100% purity, FA salt) was obtained as a yellow solid. LCMS: Rt=0.334 min, [M+H]⁺=787.2. ¹H NMR (400 MHz, METHANOL-d₄) δ=8.46 (s, 1H), 8.36 (d, J=7.6 Hz, 1H), 8.09 (s, 1H), 7.93 (d, J=1.6 Hz, 1H), 7.90-7.79 (m, 2H), 7.62 (d, J=7.6 Hz, 2H), 7.52-7.34 (m, 6H), 7.19-7.14 (m, 1H), 4.38 (s, 2H), 4.05 (s, 1H), 3.87-3.65 (m, 8H), 3.62-3.51 (m, 3H), 3.45 (s, 1H), 3.41-3.32 (m, 3H), 3.14 (s, 2H), 2.86 (s, 2H), 1.99 (s, 3H), 1.91-1.75 (m, 3H), 1.73-1.55 (m, 2H). ¹⁹F NMR (400 MHz, METHANOL-d₄) δ=−120.695.

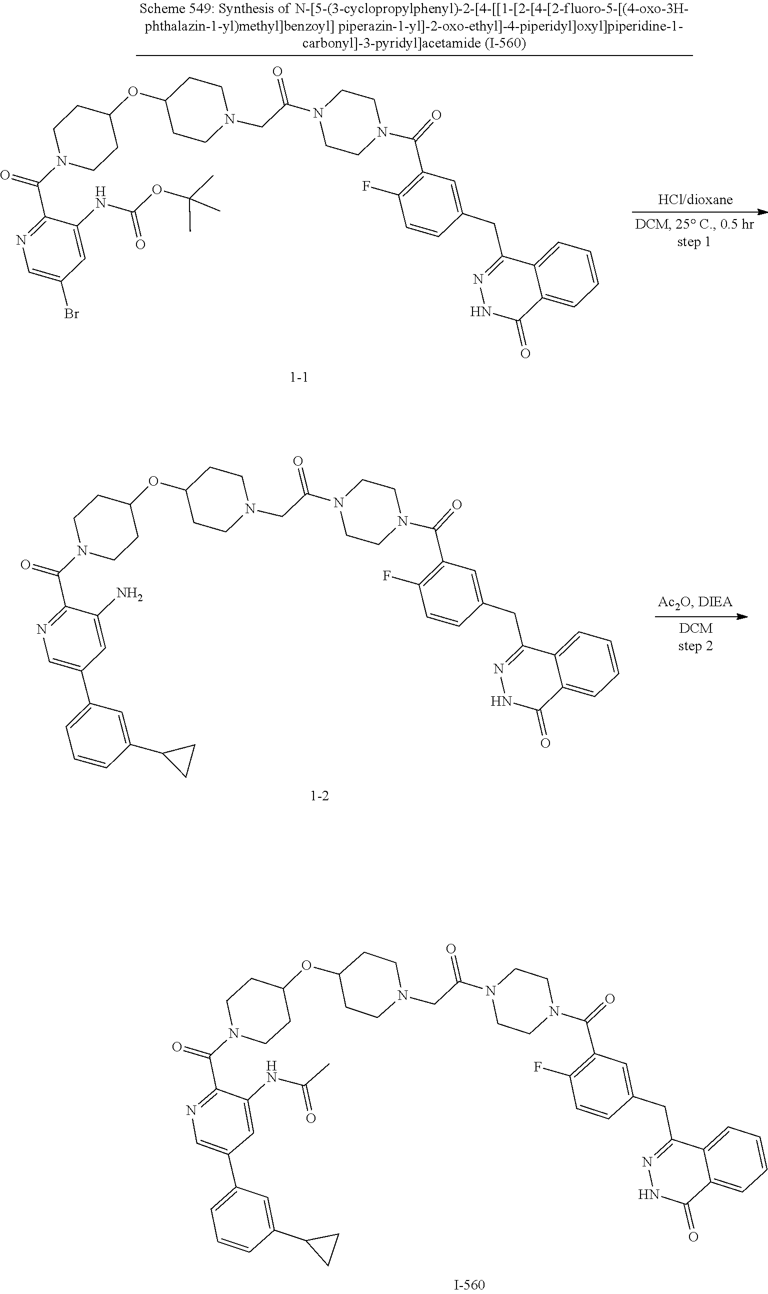 [SMILES: CC(C)(C)OC(=O)Nc1cc(Br)cnc1C(=O)N1CCC(OC2CCN(CC(=O)N3CCN(C(=O)c4cc(Cc5n[nH]c(=O)c6ccccc56)ccc4F)CC3)CC2)CC1 ; Nc1cc(-c2cccc(C3CC3)c2)cnc1C(=O)N1CCC(OC2CCN(CC(=O)N3CCN(C(=O)c4cc(Cc5n[nH]c(=O)c6ccccc56)ccc4F)CC3)CC2)CC1 ; CC(=O)Nc1cc(-c2cccc(C3CC3)c2)cnc1C(=O)N1CCC(OC2CCN(CC(=O)N3CCN(C(=O)c4cc(Cc5n[nH]c(=O)c6ccccc56)ccc4F)CC3)CC2)CC1]

### Step 1: Synthesis of Intermediate 1-2

To a solution of Intermediate 1-1 (100 mg, 107.87 μmol, 1 eq) in DCM (1 mL) was added HCl/dioxane (2 M, 0.4 mL). The mixture was stirred at 25° C. for 0.5 hr. The mixture was concentrated under reduced pressure to give Intermediate 1-2 (80 mg, 96.74 μmol, 89.69% yield) as a brown oil. LCMS (Method D): Rt: 0.369 min, [M+H]⁺=827.5.

### Step 2: Synthesis of I-560

To a solution of Intermediate 1-2 (60 mg, 72.56 μmol, 1 eq) in DCM (1 mL) was added acetyl chloride (6.83 mg, 87.07 μmol, 6.19 μL, 1.2 eq) and DIEA (18.75 mg, 145.11 μmol, 25.28 μL, 2 eq). The mixture was stirred at 25° C. for 0.5 hr. The reaction mixture was diluted with water (5 mL) and extracted with ethyl acetate (5 mL×3). The combined organic layers were washed with brine (5 mL×3) and dried over sodium sulfate, filtered and concentrated under reduced pressure to give a residue. The crude product was purified by prep-HPLC (column: CD07-Daisogel SP-100-8-ODS-PK 150*25*10 um; mobile phase: [water (NH₄HCO₃)-ACN]; gradient: 28%-588% B over 10 min) and the eluent was concentrated under reduced pressure to remove ACN and then lyophilized to give I-560 (22.99 mg, 26.46 μmol, 36.46% yield, 100% purity) as a white solid. LCMS (Method D): Rt: 0.374 min, [M+H]⁺=869.3. ¹H NMR (400 MHz, METHANOL-d₄) δ=8.61 (d, J=1.6 Hz, 1H), 8.40-8.34 (m, 1H), 8.26 (s, 1H), 7.97-7.92 (m, 1H), 7.90-7.80 (m, 2H), 7.52-7.42 (m, 2H), 7.41-7.35 (m, 3H), 7.20-7.13 (m, 2H), 4.38 (s, 2H), 4.08-3.98 (m, 1H), 3.86-3.45 (m, 11H), 3.30-3.16 (m, 4H), 2.78 (d, J=1.2 Hz, 2H), 2.26-2.25 (m, 2H), 2.17 (s, 3H), 2.05-1.83 (m, 5H), 1.73-1.54 (m, 4H), 1.06-0.97 (m, 2H), 0.80-0.74 (m, 2H). ¹⁹F NMR (376 MHz, METHANOL-d₄) δ=−120.799.

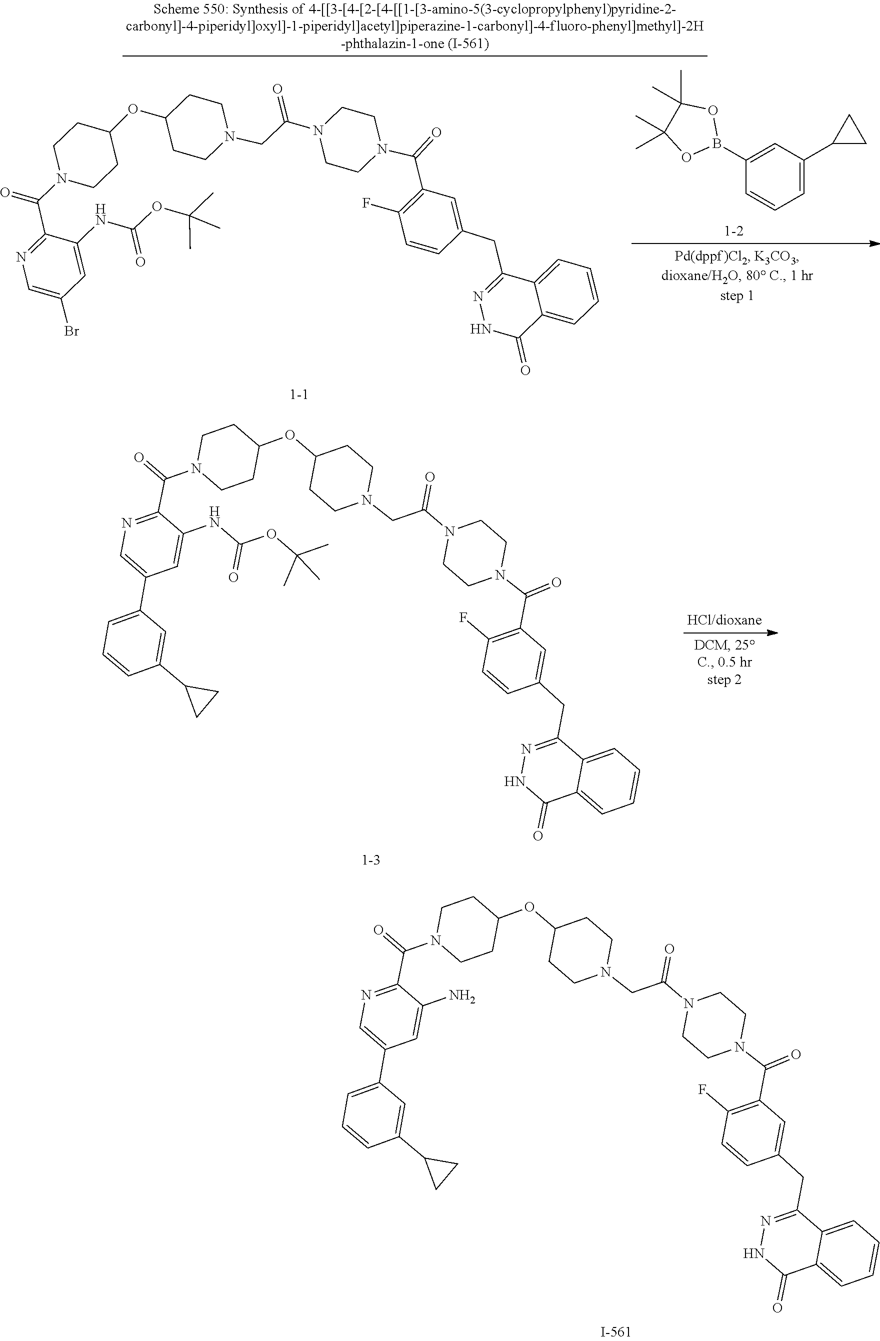 [SMILES: CC(C)(C)OC(=O)Nc1cc(Br)cnc1C(=O)N1CCC(OC2CCN(CC(=O)N3CCN(C(=O)c4cc(Cc5n[nH]c(=O)c6ccccc56)ccc4F)CC3)CC2)CC1 ; CC1(C)OB(c2cccc(C3CC3)c2)OC1(C)C ; CC(C)(C)OC(=O)Nc1cc(-c2cccc(C3CC3)c2)cnc1C(=O)N1CCC(OC2CCN(CC(=O)N3CCN(C(=O)c4cc(Cc5n[nH]c(=O)c6ccccc56)ccc4F)CC3)CC2)CC1 ; Nc1cc(-c2cccc(C3CC3)c2)cnc1C(=O)N1CCC(OC2CCN(CC(=O)N3CCN(C(=O)c4cc(Cc5n[nH]c(=O)c6ccccc56)ccc4F)CC3)CC2)CC1]

### Step 1: Synthesis of Intermediate 1-3

To a solution of Intermediate 1-1 (200 mg, 224.77 μmol, 1 eq) and Intermediate 1-2 (54.87 mg, 224.77 μmol, 1 eq) in H₂O (0.5 mL) and dioxane (1.5 mL) was added Pd(dppf)Cl₂ (16.45 mg, 22.48 μmol, 0.1 eq) and K₂CO₃ (93.19 mg, 674.30 μmol, 3 eq). The suspension was degassed and purged with N₂ 3 times. The mixture was stirred at 90° C. for 1 hr under N₂. The mixture was concentrated under reduced pressure to give Intermediate 1-3 (180 mg, 194.16 μmol, 86.38% yield) as a brown oil. LCMS (Method D): Rt: 0.431 min, [M+H]⁺=927.3.

### Step 2: Synthesis of I-561

To a solution of Intermediate 1-3 (80 mg, 86.29 μmol, 1 eq) in DCM (1 mL) was added HCl/dioxane (2 M, 0.4 mL). The mixture was stirred at 25° C. for 0.5 hr. The mixture was concentrated under reduced pressure to give a residue. The crude product was purified by reversed-phase chromatography (neutral condition) and the eluent was concentrated under reduced pressure to remove ACN and then lyophilized to give I-561 (21.57 mg, 26.08 μmol, 30.23% yield, 100% purity) as a white solid. LCMS (Method D): Rt: 0.374 min, [M+H]⁺=827.2. ¹H NMR (400 MHz, METHANOL-d₄) δ=8.38-8.30 (m, 2H), 8.07 (s, 1H), 7.95 (d, J=6.4 Hz, 1H), 7.91-7.79 (m, 2H), 7.49 (d, J=7.2 Hz, 2H), 7.45-7.36 (m, 3H), 7.21 (d, J=7.6 Hz, 1H), 7.16 (s, 1H), 4.45-4.30 (m, 4H), 3.99-3.64 (m, 8H), 3.60-3.33 (m, 9H), 3.23 (s, 1H), 2.24 (d, J=12.4 Hz, 1H), 2.13-1.94 (m, 5H), 1.90-1.65 (m, 3H), 1.06-0.99 (m, 2H), 0.79-0.76 (m, 2H). ¹⁹F NMR (376 MHz, METHANOL-d₄) δ=−120.622.

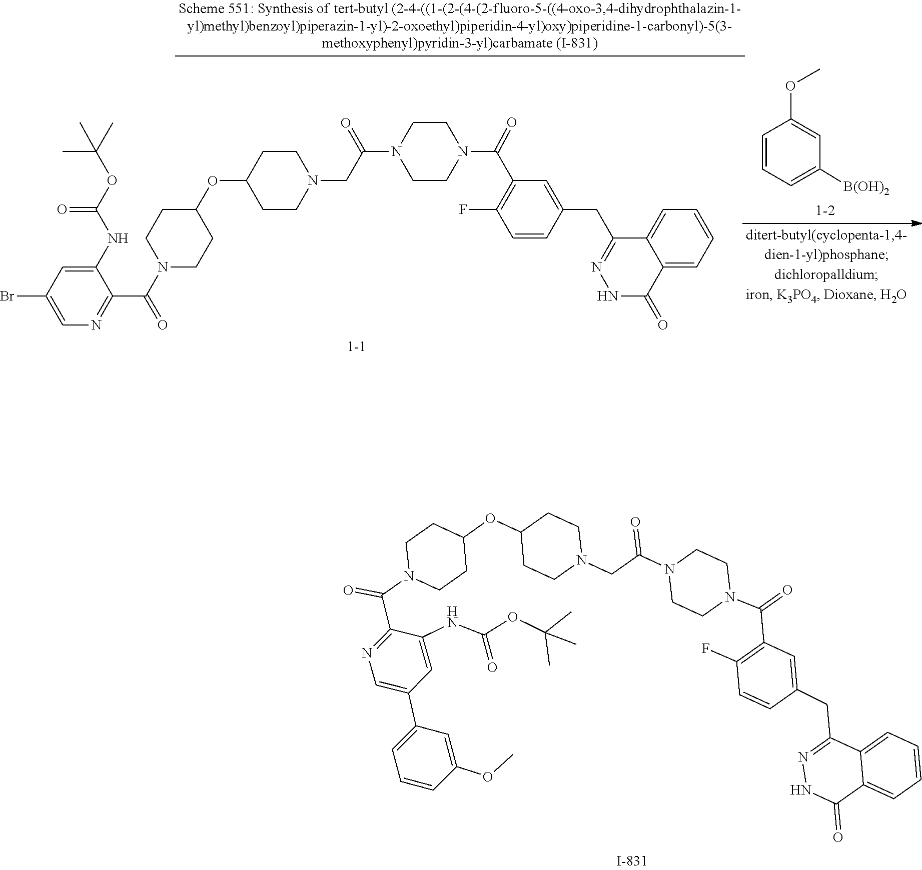 [SMILES: CC(C)(C)OC(=O)Nc1cc(Br)cnc1C(=O)N1CCC(OC2CCN(CC(=O)N3CCN(C(=O)c4cc(Cc5n[nH]c(=O)c6ccccc56)ccc4F)CC3)CC2)CC1 ; COc1cccc(B(O)O)c1 ; COc1cccc(-c2cnc(C(=O)N3CCC(OC4CCN(CC(=O)N5CCN(C(=O)c6cc(Cc7n[nH]c(=O)c8ccccc78)ccc6F)CC5)CC4)CC3)c(NC(=O)OC(C)(C)C)c2)c1]

A mixture of Intermediate 1-1 (50 mg, 56.19 μmol, 1 eq), Intermediate 1-2 (10.25 mg, 67.43 μmol, 1.2 eq), ditert-butyl(cyclopenta-1,4-dien-1-yl) phosphane; dichloropalladium;iron (3.66 mg, 5.62 μmol, 0.1 eq), K₃PO₄ (35.78 mg, 168.58 μmol, 3 eq) in dioxane (0.5 mL) and H₂O (0.1 mL) was degassed and purged with N₂ 3 times, and then the mixture was stirred at 80° C. for 1 hr under N₂ atmosphere. To the reaction mixture was added H₂O (3 mL) and then extracted with DCM (3 mL*3), the combined organic phase was washed, dried by Na₂SO₄, filtered and concentrated. The crude product was purified by reversed-phase chromatography (0.1% FA condition) and the eluent was concentrated to remove ACN and lyophilized to give product. I-831 (23.29 mg, 25.33 μmol, 45.07% yield, 99.719% purity) was obtained as a white solid. LCMS (Method D): Retention time: 0.389 min, [M+H]⁺=917.3. ¹H NMR (400 MHz, METHANOL-d₄) δ=8.55-8.50 (m, 1H), 8.43-8.30 (m, 2H), 7.99-7.91 (m, 1H), 7.91-7.80 (m, 2H), 7.49 (d, J=1.6 Hz, 1H), 7.45-7.40 (m, 1H), 7.38 (s, 1H), 7.25 (d, J=7.6 Hz, 1H), 7.21 (s, 1H), 7.16 (t, J=9.2 Hz, 1H), 7.03-7.00 (m, 1H), 4.39 (s, 2H), 4.13-4.00 (m, 1H), 3.87 (s, 3H), 3.80 (d, J=5.6 Hz, 2H), 3.74 (d, J=5.6 Hz, 1H), 3.69 (d, J=3.2 Hz, 3H), 3.65-3.46 (m, 5H), 3.40 (s, 4H), 2.95-2.78 (m, 2H), 2.54-2.26 (m, 2H), 2.01-1.85 (m, 4H), 1.74-1.59 (m, 4H), 1.53 (s, 9H). ¹⁹F NMR (376 MHz, METHANOL-d₄) δ=−120.787.

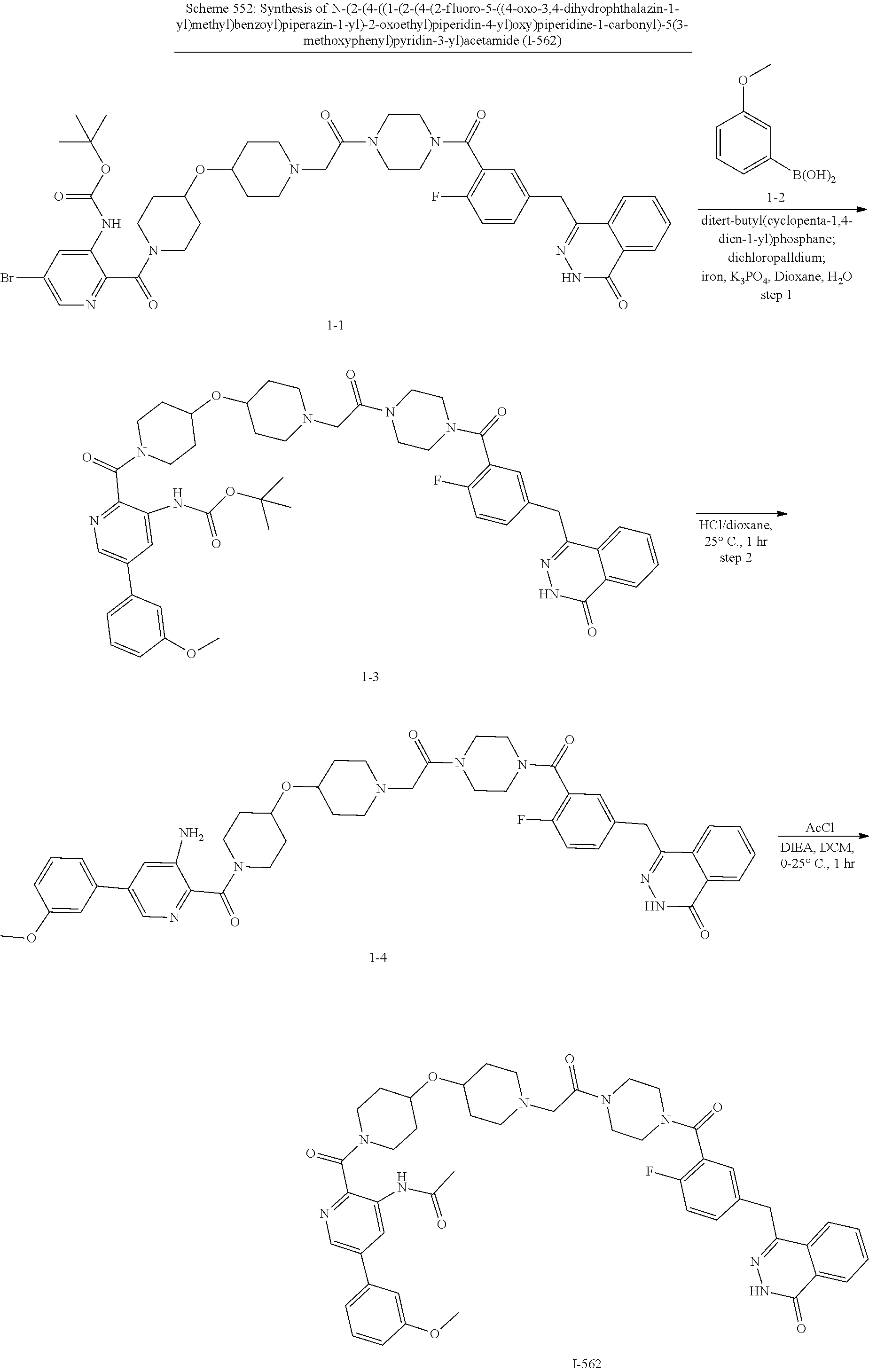

### Step 1: Synthesis of Intermediate 1-2

To a mixture of Intermediate 1-1 (200 mg, 224.77 μmol, 1 eq), Intermediate 1-2 (40.99 mg, 269.72 μmol, 1.2 eq), ditert-butyl(cyclopenta-1,4-dien-1-yl) phosphane; dichloropalladium; iron (14.65 mg, 22.48 μmol, 0.1 eq), and K₃PO₄ (143.13 mg, 674.30 μmol, 3 eq) in dioxane (2 mL) and H₂O (0.4 mL) was degassed and purged with N₂ 3 times, and then the mixture was stirred at 80° C. for 1 hr under N₂ atmosphere. To the reaction mixture was added H₂O (5 mL) and then it was extracted with DCM (5 mL*3), the combined organic phase was washed, dried by Na₂SO₄, filtered and concentrated. The crude product was purified by reversed-phase chromatography (0.1% FA condition), the eluent was concentrated to remove ACN and lyophilized to give product. Intermediate 1-3 (130 mg, 133.26 μmol, 59.29% yield, 94% purity) was obtained as a white solid. LCMS (Method D): Retention time: 0.393 min, [M+H]⁺=917.3.

### Step 2: Synthesis of Intermediate 1-3

A solution of Intermediate 1-2 (80 mg, 87.24 μmol, 1 eq) in HCl/dioxane (2 M, 800.00 μL, 18.34 eq) was stirred at 25° C. for 1 hr. The reaction mixture was concentrated under reduced pressure to give a residue. Intermediate 1-3 (80 mg, crude, HCl salt) was obtained as a white solid. LCMS (Method D): Retention time: 0.322 min, [M+H]⁺=817.3.

### Step 3: Synthesis of I-562

To a solution of Intermediate 1-3 (70 mg, 82.03 μmol, 1 eq, HCl salt) in DCM (0.7 mL) was added DIEA (31.80 mg, 246.08 μmol, 42.86 μL, 3 eq) and acetyl chloride (7.73 mg, 98.43 μmol, 7.00 μL, 1.2 eq). The mixture was stirred at 0° C. for 1 hr. To the reaction mixture was added H₂O (2 mL) and then it was extracted with DCM (2 mL*3), the combined organic phase was washed, dried by Na₂SO₄, filtered and concentrated. The residue was purified by prep-HPLC (column: CD01-Phenomenex luna C18 150*25*10 um; mobile phase: [water (FA)-ACN]; gradient: 10%-40% B over 10 min). The eluent was concentrated to remove ACN and lyophilized to give product. I-562 (17.24 mg, 19.05 μmol, 23.22% yield, 100.000% purity, FA salt) was obtained as a white solid. LCMS (Method D): Retention time: 0.338 min, [M+H]⁺=859.3. ¹H NMR (400 MHz, METHANOL-d₄) δ=8.63 (d, J=1.6 Hz, 1H), 8.47 (s, 1H), 8.40-8.32 (m, 1H), 8.24 (d, J=1.6 Hz, 1H), 8.00-7.92 (m, 1H), 7.91-7.75 (m, 2H), 7.54-7.46 (m, 1H), 7.46-7.40 (m, 1H), 7.40-7.34 (m, 1H), 7.29-7.20 (m, 2H), 7.17 (t, J=8.8 Hz, 1H), 7.06-6.99 (m, 1H), 4.39 (s, 2H), 4.09-3.97 (m, 1H), 3.87 (s, 3H), 3.80 (d, J=4.8 Hz, 2H), 3.75 (s, 1H), 3.70-3.60 (m, 4H), 3.60-3.44 (m, 5H), 3.38-3.34 (m, 1H), 3.28 (s, 1H), 3.06-2.87 (m, 2H), 2.72-2.43 (m, 2H), 2.17 (s, 3H), 2.02-1.86 (m, 4H), 1.80-1.60 (m, 4H). ¹⁹F NMR (376 MHz, METHANOL-d₄) δ=−120.787.

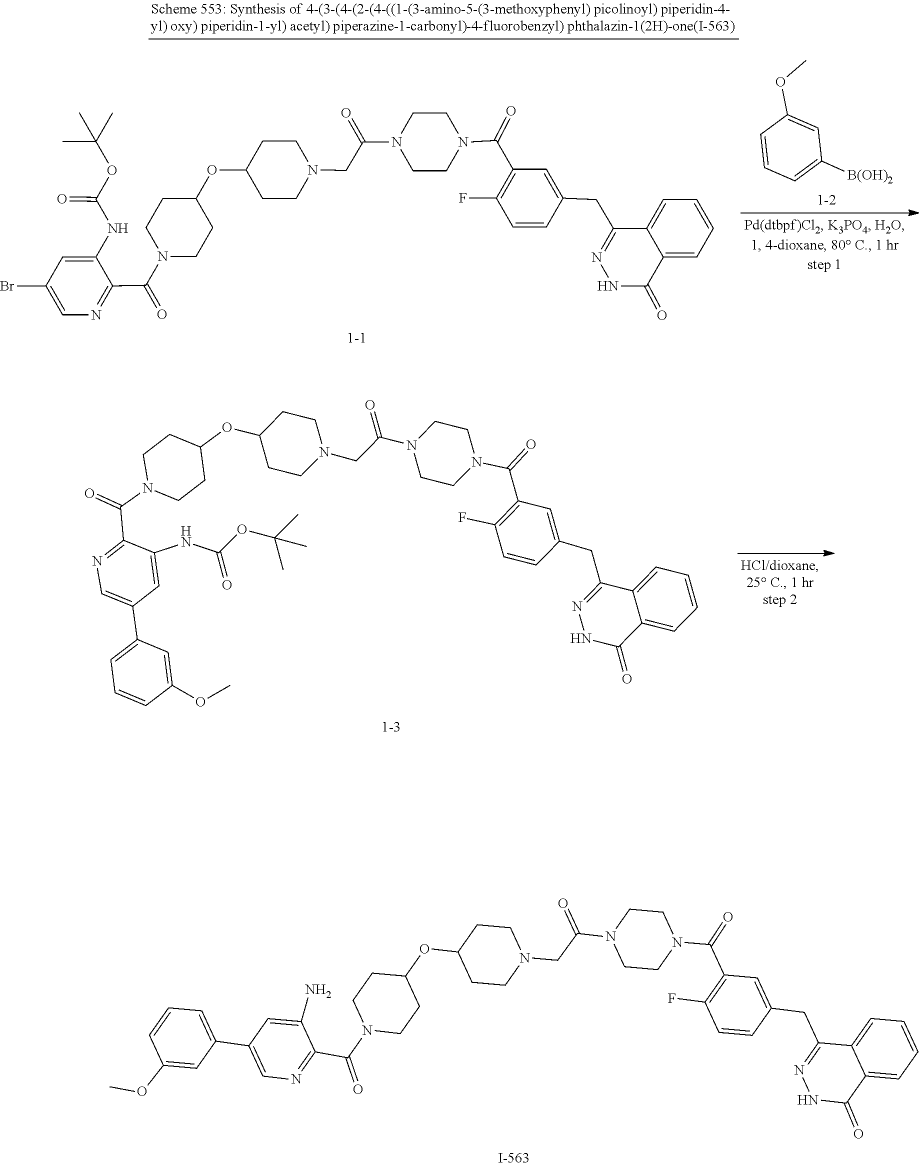 [SMILES: CC(C)(C)OC(=O)Nc1cc(Br)cnc1C(=O)N1CCC(OC2CCN(CC(=O)N3CCN(C(=O)c4cc(Cc5n[nH]c(=O)c6ccccc56)ccc4F)CC3)CC2)CC1 ; COc1cccc(B(O)O)c1 ; COc1cccc(-c2cnc(C(=O)N3CCC(OC4CCN(CC(=O)N5CCN(C(=O)c6cc(Cc7n[nH]c(=O)c8ccccc78)ccc6F)CC5)CC4)CC3)c(NC(=O)OC(C)(C)C)c2)c1 ; COc1cccc(-c2cnc(C(=O)N3CCC(OC4CCN(CC(=O)N5CCN(C(=O)c6cc(Cc7n[nH]c(=O)c8ccccc78)ccc6F)CC5)CC4)CC3)c(N)c2)c1]

### Step 1: Synthesis of Intermediate 1-2

A mixture of Intermediate 1-1 (200 mg, 224.77 μmol, 1 eq), Intermediate 1-2 (40.99 mg, 269.72 μmol, 1.2 eq), Pd(dtbpf)Cl₂ (14.65 mg, 22.48 μmol, 0.1 eq), and K₃PO₄ (143.13 mg, 674.30 μmol, 3 eq) in dioxane (2 mL) and H₂O (0.4 mL) was degassed and purged with N₂ 3 times, and then the mixture was stirred at 80° C. for 1 hr under N₂ atmosphere. To the reaction mixture was added H₂O (5 mL) and then it was extracted with DCM (5 mL*3), the combined organic phase was washed, dried by Na₂SO₄, filtered and concentrated. The crude product was purified by reversed-phase chromatography (0.1% FA condition), and the eluent was concentrated to remove ACN and lyophilized to give product. Intermediate 1-3 (130 mg, 133.26 μmol, 59.29% yield, 94% purity) was obtained as a white solid. LCMS (Method D): Retention time: 0.391 min, [M+H]⁺=917.3.

### Step 2: Synthesis of I-563

A solution of Intermediate 1-3 (30 mg, 32.71 μmol, 1 eq) in HCl/dioxane (2 M, 300.00 μL, 18.34 eq) was stirred at 25° C. for 1 hr. The reaction mixture was concentrated under reduced pressure to give a residue. The crude was used for the next step. To the product was added H₂O (10 mL) and ACN (3 mL) and then it was lyophilized to give product. I-563 (24.96 mg, 28.41 μmol, 86.85% yield, 97.142% purity, HCl salt) was obtained as a white solid. LCMS (Method D): Retention time: 0.331 min, [M+H]⁺=817.3. ¹H NMR (400 MHz, METHANOL-d₄) δ=8.37-8.35 (m, 1H), 8.25 (d, J=1.6 Hz, 1H), 7.94 (d, J=6.8 Hz, 1H), 7.91-7.80 (m, 3H), 7.55-7.48 (m, 1H), 7.48-7.42 (m, 1H), 7.40-7.33 (m, 1H), 7.28-7.21 (m, 2H), 7.17 (t, J=9.2 Hz, 1H), 7.07 (d, J=8.4 Hz, 1H), 4.39 (s, 2H), 4.33 (d, J=4.0 Hz, 1H), 4.26 (d, J=5.2 Hz, 1H), 3.96 (s, 1H), 3.87 (s, 4H), 3.82 (s, 2H), 3.77 (d, J=4.4 Hz, 1H), 3.72 (d, J=3.2 Hz, 1H), 3.69-3.63 (m, 1H), 3.57-3.44 (m, 5H), 3.40-3.33 (m, 4H), 3.20-3.08 (m, 1H), 2.28-2.16 (m, 1H), 2.12-2.01 (m, 3H), 2.00-1.91 (m, 2H), 1.90-1.78 (m, 1H), 1.78-1.64 (m, 2H). ¹⁹F NMR (376 MHz, METHANOL-d₄) δ=−120.764.

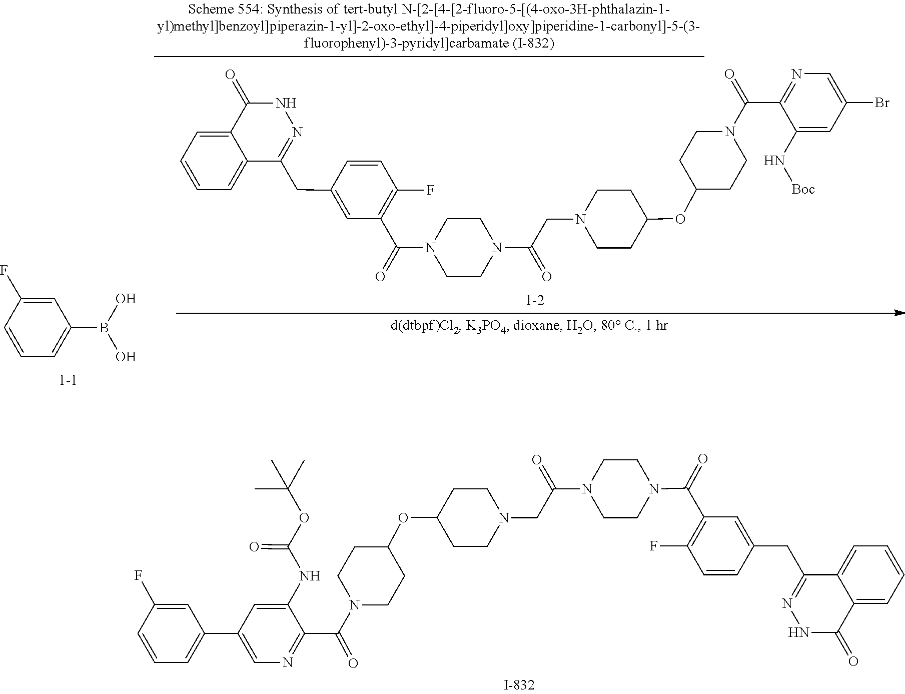 [SMILES: OB(O)c1cccc(F)c1 ; CC(C)(C)OC(=O)Nc1cc(Br)cnc1C(=O)N1CCC(OC2CCN(CC(=O)N3CCN(C(=O)c4cc(Cc5n[nH]c(=O)c6ccccc56)ccc4F)CC3)CC2)CC1 ; CC(C)(C)OC(=O)Nc1cc(-c2cccc(F)c2)cnc1C(=O)N1CCC(OC2CCN(CC(=O)N3CCN(C(=O)c4cc(Cc5n[nH]c(=O)c6ccccc56)ccc4F)CC3)CC2)CC1]

A mixture of Intermediate 1-1 (15 mg, 107.20 μmol, 1 eq), Intermediate 1-2 (95.39 mg, 107.20 μmol, 1 eq), ditert-butyl(cyclopenta-1,4-dien-1-yl)phosphane;dichloropalladium;iron (6.99 mg, 10.72 μmol, 0.1 eq), and K₃PO₄ (45.51 mg, 214.41 μmol, 2 eq) in dioxane (0.5 mL) and H₂O (0.1 mL) was degassed and purged with N₂ 3 times, and then the mixture was stirred at 80° C. for 1 hr under N₂ atmosphere. The residue was diluted with H₂O (5 mL) and extracted with EA (5 mL*3), dried over Na₂SO₄, filtered and concentrated under reduced pressure to give a residue. The crude product was purified by reversed-phase chromatography (0.1% FA condition). The eluent was concentrated under reduced pressure to remove ACN, then lyophilized to afford product. I-832 (65 mg, 68.35 μmol, 63.75% yield, 100% purity, FA salt) was obtained as a yellow solid. LCMS (Method D): Rt=0.414 min, [M+H]⁺=905.2. ¹H NMR (400 MHz, METHANOL-d₄) δ=8.56 (s, 1H), 8.43 (br s, 1H), 8.42 (d, J=1.2 Hz, 1H), 8.37 (d, J=7.6 Hz, 1H), 8.00-7.91 (m, 1H), 7.91-7.80 (m, 2H), 7.58-7.44 (m, 4H), 7.37 (br t, J=6.4 Hz, 1H), 7.25-7.12 (m, 2H), 4.39 (s, 2H), 4.07-4.03 (m, 1H), 3.83-3.73 (m, 3H), 3.73-3.62 (m, 5H), 3.62-3.44 (m, 5H), 3.39-3.33 (m, 2H), 3.10-2.96 (m, 2H), 2.81-2.56 (m, 2H), 2.02-1.90 (m, 4H), 1.82-1.65 (m, 4H), 1.53 (s, 9H). ¹⁹F NMR: (376 MHz, METHANOL-d₄) δ=−114.197, 120.779.

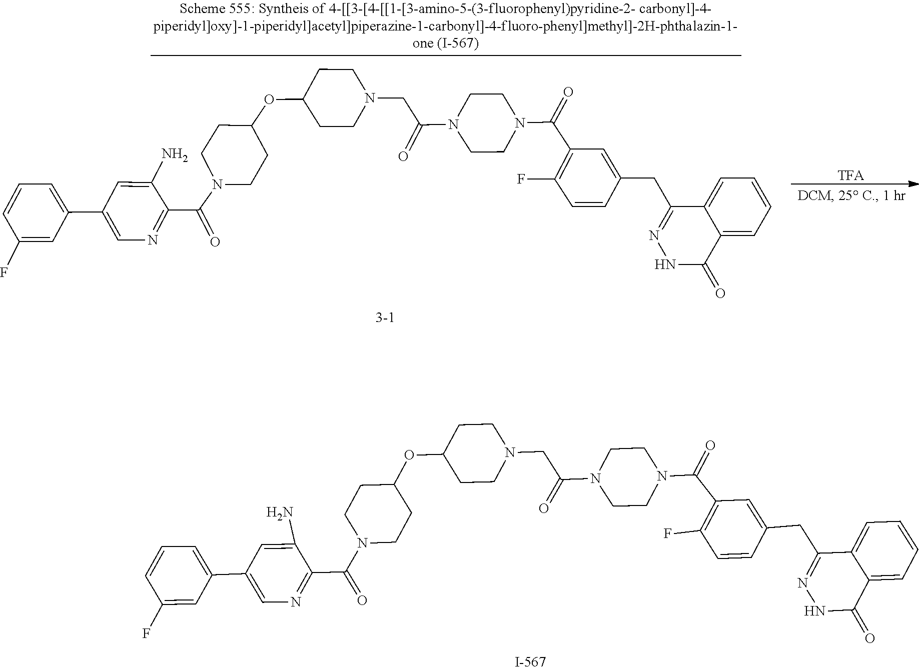 [SMILES: Nc1cc(-c2cccc(F)c2)cnc1C(=O)N1CCC(OC2CCN(CC(=O)N3CCN(C(=O)c4cc(Cc5n[nH]c(=O)c6ccccc56)ccc4F)CC3)CC2)CC1 ; Nc1cc(-c2cccc(F)c2)cnc1C(=O)N1CCC(OC2CCN(CC(=O)N3CCN(C(=O)c4cc(Cc5n[nH]c(=O)c6ccccc56)ccc4F)CC3)CC2)CC1 ; "TFA"]

A solution of intermediate 2-1 (50 mg, 55.25 μmol, 1 eq) in DCM (0.4 mL) and TFA (0.2 mL) was stirred at 25° C. for 0.5 hr. The residue was concentrated under reduced pressure then washed by PE (1 mL*3) to give a residue. The crude product was purified by reversed-phase chromatography (0.1% TFA condition). The eluent was concentrated under reduced pressure to remove ACN, then lyophilized to afford product. I-567 (25 mg, 26.29 μmol, 47.59% yield, 96.638% purity, TFA salt) was obtained as a white solid. LCMS (Method D): Rt=0.331 min, [M+H]⁺=805.3. ¹H NMR (400 MHz, METHANOL-d₄) δ=8.41-8.33 (m, 1H), 8.14 (br s, 1H), 7.95 (br d, J=6.8 Hz, 1H), 7.92-7.81 (m, 2H), 7.56-7.45 (m, 4H), 7.44-7.33 (m, 3H), 7.18 (brt, J=8.8 Hz, 2H), 4.40 (s, 2H), 4.33-4.17 (m, 2H), 3.97 (br s, 1H), 3.87-3.64 (m, 6H), 3.60-3.41 (m, 5H), 3.35 (br d, J=10.0 Hz, 4H), 3.18-3.07 (m, 1H), 2.27-1.85 (m, 6H), 1.77-1.57 (m, 2H). ¹⁹F NMR: (377 MHz, METHANOL-d₄) δ=−114.392, −120.764.

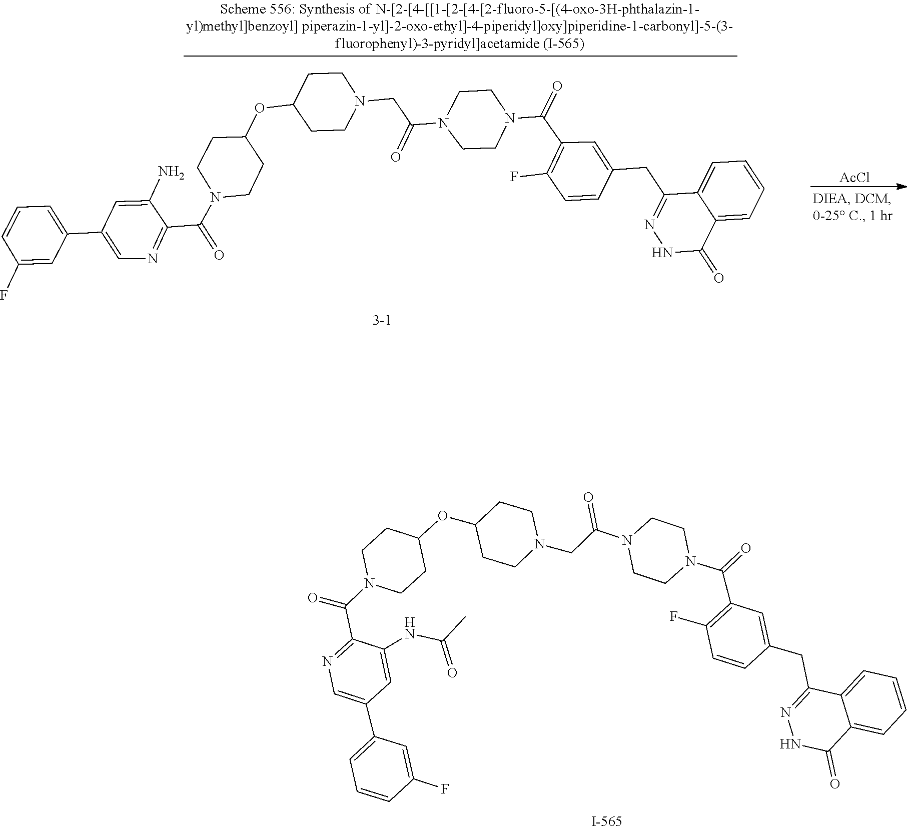 [SMILES: "AcCl" ; Nc1cc(-c2cccc(F)c2)cnc1C(=O)N1CCC(OC2CCN(CC(=O)N3CCN(C(=O)c4cc(Cc5n[nH]c(=O)c6ccccc56)ccc4F)CC3)CC2)CC1 ; CC(=O)Nc1cc(-c2cccc(F)c2)cnc1C(=O)N1CCC(OC2CCN(CC(=O)N3CCN(C(=O)c4cc(Cc5n[nH]c(=O)c6ccccc56)ccc4F)CC3)CC2)CC1]

### Step 1: Synthesis of I-565

To a solution of intermediate 3-1 (80 mg, 99.39 μmol, 1 eq) in DCM (0.8 mL) was added DIEA (25.69 mg, 198.79 μmol, 34.62 μL, 2 eq) and acetyl chloride (7.80 mg, 99.39 μmol, 7.07 μL, 1 eq). The mixture was stirred at 25° C. for 1 hr. The residue was diluted with H₂O (5 mL) and extracted with DCM (5 mL*3), dried over Na₂SO₄, filtered and concentrated under reduced pressure to give a residue. The crude product was purified by reversed-phase chromatography (0.1% TFA condition). The eluent was concentrated under reduced pressure to remove ACN and lyophilized to afford product. I-565 (33 mg, 33.78 μmol, 33.98% yield, 98.359% purity, TFA salt) was obtained as a white solid. LCMS (Method D): Rt=0.352 min, [M+H]⁺=847.3. ¹H NMR (400 MHz, METHANOL-d₄) δ=8.66 (s, 1H), 8.37 (br d, J=7.6 Hz, 1H), 8.27 (s, 1H), 7.94 (br d, J=3.2 Hz, 1H), 7.91-7.79 (m, 2H), 7.59-7.43 (m, 4H), 7.37 (br t, J=5.6 Hz, 1H), 7.25-7.11 (m, 2H), 4.39 (s, 2H), 4.01 (br d, J=6.4 Hz, 1H), 3.86-3.74 (m, 3H), 3.66 (br s, 5H), 3.61-3.44 (m, 5H), 3.35 (br s, 1H), 3.10-2.95 (m, 2H), 2.82-2.51 (m, 2H), 2.17 (s, 3H), 2.05-1.56 (m, 9H). ¹⁹F NMR (377 MHz, METHANOL-d₄) δ=−76.894, −114.114, −120.757.

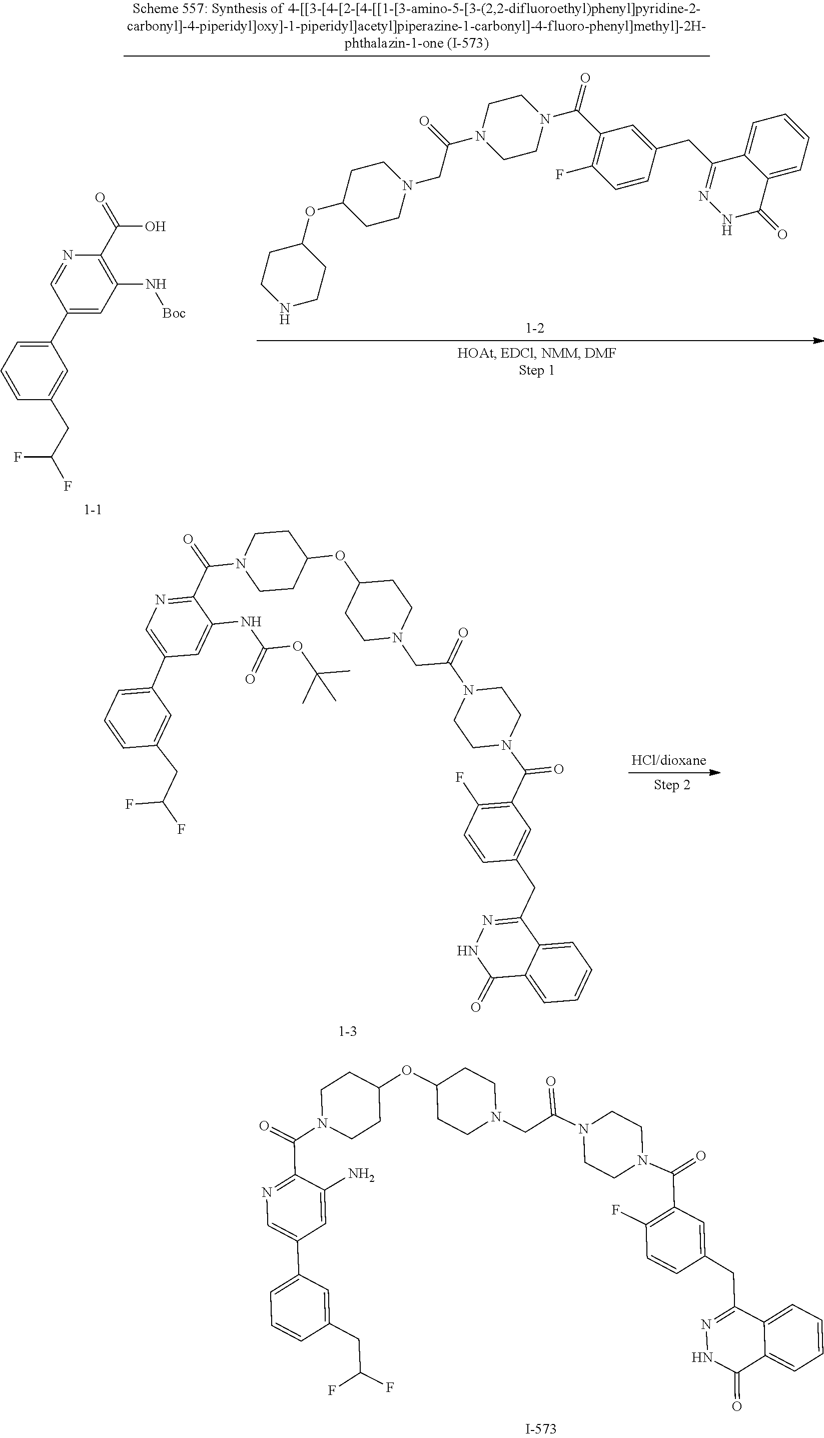 [SMILES: CC(C)(C)OC(=O)Nc1cc(-c2cccc(CC(F)F)c2)cnc1C(=O)O ; O=C(CN1CCC(OC2CCNCC2)CC1)N1CCN(C(=O)c2cc(Cc3n[nH]c(=O)c4ccccc34)ccc2F)CC1 ; CC(C)(C)OC(=O)Nc1cc(-c2cccc(CC(F)F)c2)cnc1C(=O)N1CCC(OC2CCN(CC(=O)N3CCN(C(=O)c4cc(Cc5n[nH]c(=O)c6ccccc56)ccc4F)CC3)CC2)CC1 ; Nc1cc(-c2cccc(CC(F)F)c2)cnc1C(=O)N1CCC(OC2CCN(CC(=O)N3CCN(C(=O)c4cc(Cc5n[nH]c(=O)c6ccccc56)ccc4F)CC3)CC2)CC1]

### Step 1: Synthesis of Intermediate 1-3

To a mixture of intermediate 1-1 (30 mg, 79.29 μmol, 1 eq) and intermediate 1-2 (46.83 mg, 79.29 μmol, 1 eq) in DMF (1 mL) was added HOAt (10.79 mg, 79.29 μmol, 11.09 μL, 1 eq), EDCI (45.60 mg, 237.86 μmol, 3 eq) and NMM (40.10 mg, 396.44 μmol, 43.59 μL, 5 eq), then the mixture was stirred at 40° C. for 1 hour. The reaction mixture was diluted with H₂O (10 mL) and extracted with EA (5 mL*3). The combined organic layers were concentrated under reduced pressure to give Intermediate 1-3 (50 mg, 52.57 μmol, 66.31% yield) as a yellow solid. LCMS: Rt=0.416 min, [M+H]⁺=951.3.

### Step 2: Synthesis of I-573

To a mixture of intermediate 1-3 (50 mg, 52.57 μmol, 1 eq) in DCM (1 mL) was added HCl/dioxane (2 M, 131.43 μL, 5 eq), then the mixture was stirred at 20° C. for 1 hour. To the reaction mixture was added NaHCO₃ (aq) (about 10 mL) to adjust the pH to 7-8, and the mixture was extracted with DCM (10 mL*3). The combined organic layers were concentrated under reduced pressure to give a residue. The crude product was purified by reversed-phase chromatography (0.1% NH₃·H₂O condition). The eluent was concentrated under reduced pressure to remove ACN and then lyophilized to give I-573 (19.84 mg, 23.32 μmol, 44.35% yield, 100% purity) as a white solid. LCMS (Method D): Rt=0.352 min, [M+H]⁺=851.2. ¹H NMR (400 MHz, METHANOL-d₄) δ=8.41-8.33 (m, 1H), 8.12-8.07 (m, 1H), 7.99-7.92 (m, 1H), 7.90-7.81 (m, 2H), 7.55 (d, J=2.0 Hz, 2H), 7.50-7.43 (m, 3H), 7.40-7.33 (m, 2H), 7.19-7.13 (m, 1H), 6.24-5.88 (m, 1H), 4.38 (s, 2H), 4.12-4.02 (m, 1H), 3.86-3.60 (m, 7H), 3.59-3.49 (m, 4H), 3.38 (d, J=2.8 Hz, 2H), 3.28 (d, J=4.4 Hz, 1H), 3.26-3.22 (m, 2H), 3.19 (d, J=3.6 Hz, 1H), 2.77 (s, 2H), 2.33-2.21 (m, 2H), 1.98-1.84 (m, 4H), 1.69-1.53 (m, 4H). ¹⁹F NMR (376 MHz, METHANOL-d₄) δ=−116.927, −120.792.

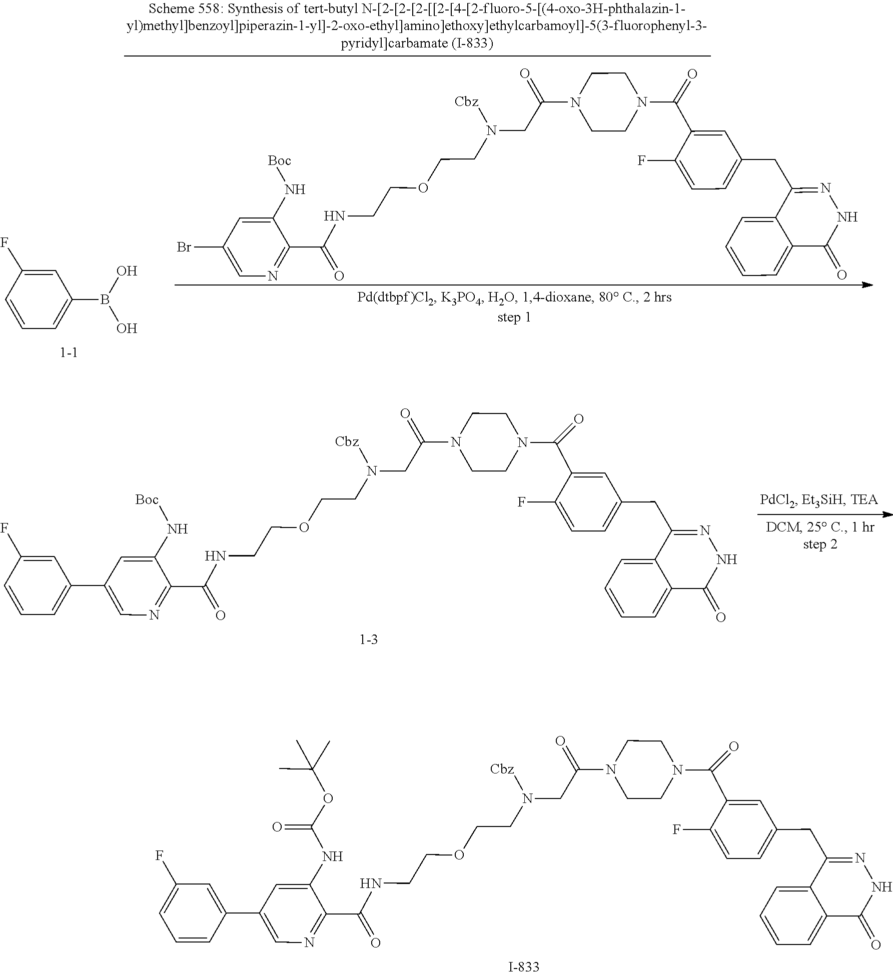 [SMILES: OB(O)c1cccc(F)c1 ; CC(C)(C)OC(=O)Nc1cc(Br)cnc1C(=O)NCCOCCN(CC(=O)N1CCN(C(=O)c2cc(Cc3n[nH]c(=O)c4ccccc34)ccc2F)CC1)C(=O)OCc1ccccc1 ; CC(C)(C)OC(=O)Nc1cc(-c2cccc(F)c2)cnc1C(=O)NCCOCCN(CC(=O)N1CCN(C(=O)c2cc(Cc3n[nH]c(=O)c4ccccc34)ccc2F)CC1)C(=O)OCc1ccccc1 ; CC(C)(C)OC(=O)Nc1cc(-c2cccc(F)c2)cnc1C(=O)NCCOCCN(CC(=O)N1CCN(C(=O)c2cc(Cc3n[nH]c(=O)c4ccccc34)ccc2F)CC1)C(=O)OCc1ccccc1]

### Step 1: Synthesis of Intermediate 1-3

To a solution of Intermediate 1-1 (53.37 mg, 381.43 μmol, 1.2 eq) and Intermediate 1-2 (300 mg, 317.86 μmol, 1 eq) in 1,4-dioxane (3 mL) and H₂O (0.6 mL) was added Pd(dtbpf)Cl₂ (62.15 mg, 95.36 μmol, 0.3 eq) and K₃PO₄ (202.41 mg, 953.58 μmol, 3 eq). The mixture was stirred at 80° C. for 2 hrs. The reaction mixture was concentrated under reduced pressure to give a residue. The residue was purified by flash silica gel chromatography (ISCO; 20 g SepaFlash Silica Flash Column, Eluent of 0˜89% Ethylacetate/MeOH @35 mL/min) and concentrated under reduced pressure to give a residue. Intermediate 1-3 (340 mg, crude) was obtained as a yellow solid. LCMS (Method D): Rt=0.616 min, [M+H]⁺=959.3.

### Step 2: Synthesis of I-833

To a solution of Intermediate 1-3 (40 mg, 41.71 μmol, 1 eq) in DCM (0.4 mL) was added PdCl₂ (3.70 mg, 20.86 μmol, 0.5 eq), Et₃SiH (58.20 mg, 500.52 μmol, 79.94 μL, 12 eq) and TEA (12.66 mg, 125.13 μmol, 17.42 μL, 3 eq). The mixture was stirred at 25° C. for 1 hr. The reaction mixture was concentrated under reduced pressure to give a residue. The crude product was purified by reversed-phase chromatography (0.1% FA condition), and lyophilized to give the product. I-833 (22.4 mg, 25.72 μmol, 61.67% yield, FA salt) was obtained as a white solid. LCMS (Method D): Rt=0.481 min, [M+H]⁺=825.4. ¹H NMR (400 MHz, METHANOL-d₄) δ=9.02 (s, 1H), 8.55-8.51 (m, 1H), 8.51-8.45 (m, 1H), 8.36 (d, J=7.6 Hz, 1H), 7.96-7.90 (m, 1H), 7.90-7.77 (m, 2H), 7.59-7.40 (m, 4H), 7.34 (d, J=3.2 Hz, 1H), 7.25-7.08 (m, 2H), 4.37 (s, 2H), 3.76-3.57 (m, 11H), 3.49 (d, J=14.0 Hz, 2H), 3.34 (s, 1H), 3.26-3.20 (m, 1H), 2.90 (s, 2H), 1.54 (s, 9H), 1.41-1.19 (m, 1H). ¹⁹F NMR (376 MHz, METHANOL-d₄) δ=−114.090, −120.672.

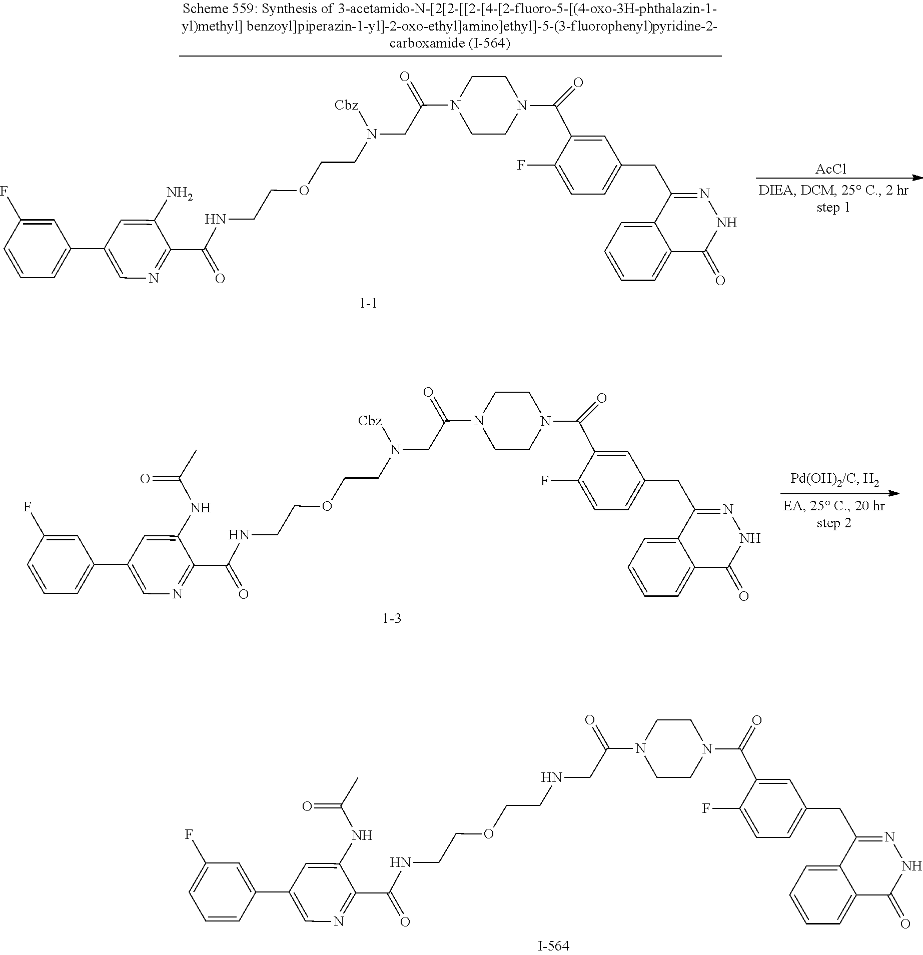 [SMILES: "AcCl" ; Nc1cc(-c2cccc(F)c2)cnc1C(=O)NCCOCCN(CC(=O)N1CCN(C(=O)c2cc(Cc3n[nH]c(=O)c4ccccc34)ccc2F)CC1)C(=O)OCc1ccccc1 ; CC(=O)Nc1cc(-c2cccc(F)c2)cnc1C(=O)NCCOCCN(CC(=O)N1CCN(C(=O)c2cc(Cc3n[nH]c(=O)c4ccccc34)ccc2F)CC1)C(=O)OCc1ccccc1 ; CC(=O)Nc1cc(-c2cccc(F)c2)cnc1C(=O)NCCOCCNCC(=O)N1CCN(C(=O)c2cc(Cc3n[nH]c(=O)c4ccccc34)ccc2F)CC1]

### Step 1: Synthesis of Intermediate 1-2

To a solution of Intermediate 1-1 (200 mg, 232.86 μmol, 1 eq) and acetyl chloride (20.11 mg, 256.15 μmol, 18.21 μL, 1.1 eq) in DCM (2 mL) was added DIEA (90.29 mg, 698.58 μmol, 121.68 μL, 3 eq). The mixture was stirred at 25° C. for 2 hr. The reaction mixture was filtered and concentrated under reduced pressure to give a residue. The residue was purified by flash silica gel chromatography (ISCO; 12 g SepaFlash Silica Flash Column, Eluent of 0˜70% Ethylacetate/MeOH @35 mL/min) and concentrated under reduced pressure to give a residue. Intermediate 1-2 (182 mg, 202.02 μmol, 86.75% yield) was obtained as a yellow solid. LCMS (Method D): Rt=0.482 min, [M+H]⁺=901.4.

### Step 2: Synthesis of I-564

To a solution of Intermediate 1-2 (182 mg, 202.02 μmol, 1 eq) in ethyl acetate (2 mL) was added Pd(OH)₂/C (20%, 95 mg) under N₂ atmosphere. The suspension was degassed and purged with H₂ 3 times. The mixture was stirred under H₂ (15 Psi) at 25° C. for 20 hrs. The reaction mixture was filtered and concentrated under reduced pressure to give a residue. The crude product was purified by reversed-phase chromatography (0.1% TFA condition) and lyophilized to give product. I-564 (20.73 mg, 23.54 μmol, 11.65% yield, 100% purity, TFA salt) was obtained as a white solid. LCMS (Method D): Rt=0.349 min, [M+H]⁺=767.2. ¹H NMR (400 MHz, METHANOL-d₄) δ=9.27 (s, 1H), 8.58 (d, J=6.4 Hz, 1H), 8.36 (d, J=7.6 Hz, 1H), 7.97-7.90 (m, 1H), 7.89-7.80 (m, 2H), 7.56-7.43 (m, 4H), 7.33 (t, J=6.0 Hz, 1H), 7.25-7.12 (m, 2H), 4.37 (s, 2H), 4.15 (s, 1H), 4.08 (s, 1H), 3.83 (d, J=3.6 Hz, 2H), 3.79-3.63 (m, 8H), 3.51-3.43 (m, 3H), 3.38 (s, 1H), 3.27 (s, 2H), 2.25 (d, J=3.2 Hz, 3H). ¹⁹F NMR (376 MHz, METHANOL-d₄) δ =−77.009, −114.000, −120.665.

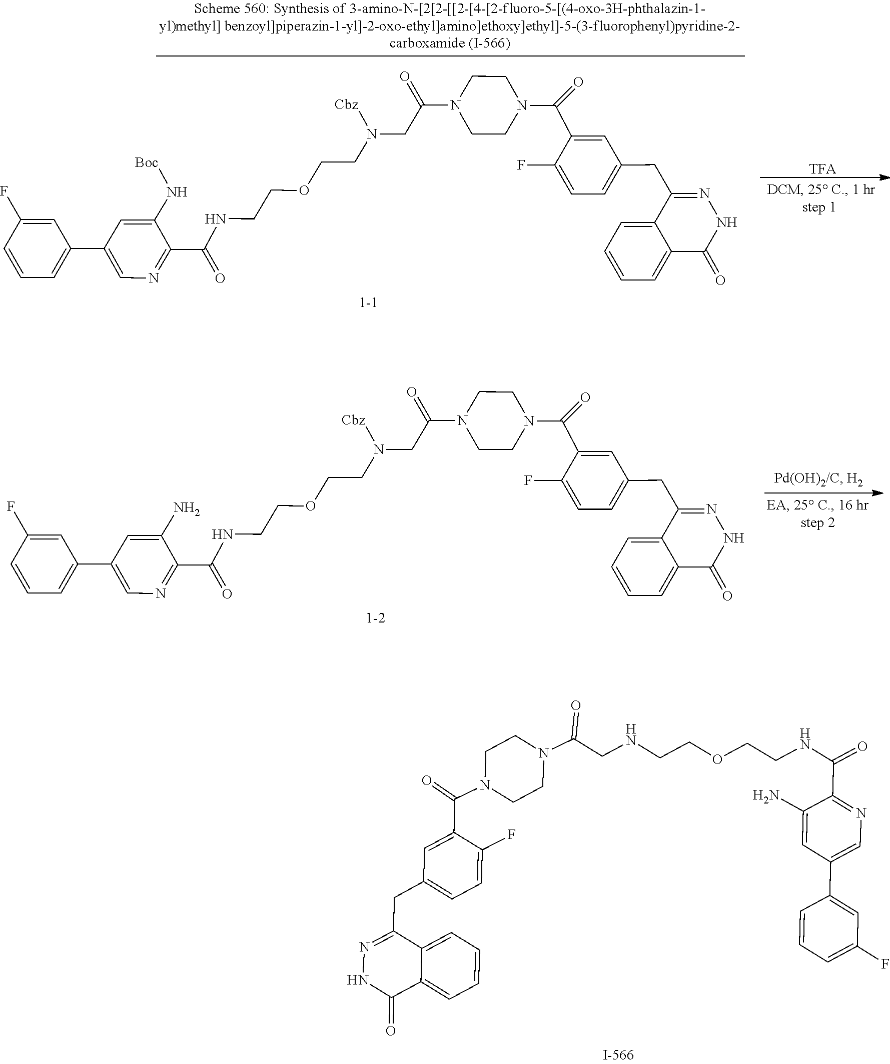 [SMILES: CC(C)(C)OC(=O)Nc1cc(-c2cccc(F)c2)cnc1C(=O)NCCOCCN(CC(=O)N1CCN(C(=O)c2cc(Cc3n[nH]c(=O)c4ccccc34)ccc2F)CC1)C(=O)OCc1ccccc1 ; Nc1cc(-c2cccc(F)c2)cnc1C(=O)NCCOCCN(CC(=O)N1CCN(C(=O)c2cc(Cc3n[nH]c(=O)c4ccccc34)ccc2F)CC1)C(=O)OCc1ccccc1 ; "TFA" ; Nc1cc(-c2cccc(F)c2)cnc1C(=O)NCCOCCNCC(=O)N1CCN(C(=O)c2cc(Cc3n[nH]c(=O)c4ccccc34)ccc2F)CC1]

### Step 1: Synthesis of Intermediate 1-2

To a solution of Intermediate 1-1 (200 mg, 208.55 μmol, 1 eq) in DCM (2 mL) was added TFA (614.00 mg, 5.38 mmol, 0.4 mL, 25.82 eq). The mixture was stirred at 25° C. for 1 hr. The reaction mixture was filtered and concentrated under reduced pressure to give a residue. The crude product was used in the next step without further purification. Intermediate 1-2 (400 mg, crude) was obtained as a yellow oil. LCMS (Method D): Rt=0.496 min, [M+H]⁺=859.4.

### Step 2: Synthesis of I-566

To a solution of intermediate 1-2 (80 mg, 93.14 μmol, 1 eq) in ethyl acetate (0.8 mL) was added Pd(OH)₂/C (20%, 40 mg) under N₂ atmosphere. The suspension was degassed and purged with H₂ 3 times. The mixture was stirred under H₂ (15 Psi) at 25° C. for 16 hrs. The reaction mixture was filtered and concentrated under reduced pressure to give a residue. The crude product was purified by reversed phase chromatography (0.1% TFA condition) and lyophilized to give product. I-566 (11.47 mg, 13.59 μmol, 14.59% yield, 99.395% purity, TFA salt) was obtained as a white solid. LCMS (Method D): Rt=0.402 min, [M+H]⁺=725.3. ¹H NMR (400 MHz, METHANOL-d₄) δ=8.36 (d, J=7.6 Hz, 1H), 8.08 (d, J=15.6 Hz, 1H), 7.93 (s, 1H), 7.89-7.80 (m, 2H), 7.47 (d, J=14.8 Hz, 3H), 7.40-7.31 (m, 3H), 7.15 (s, 2H), 4.37 (s, 2H), 4.16 (s, 1H), 4.09 (s, 1H), 3.86-3.58 (m, 11H), 3.49 (d, J=19.2 Hz, 3H), 3.24-3.08 (m, 2H). ¹⁹F NMR (376 MHz, METHANOL-d₄) δ=−77.001, −114.353, −120.635.

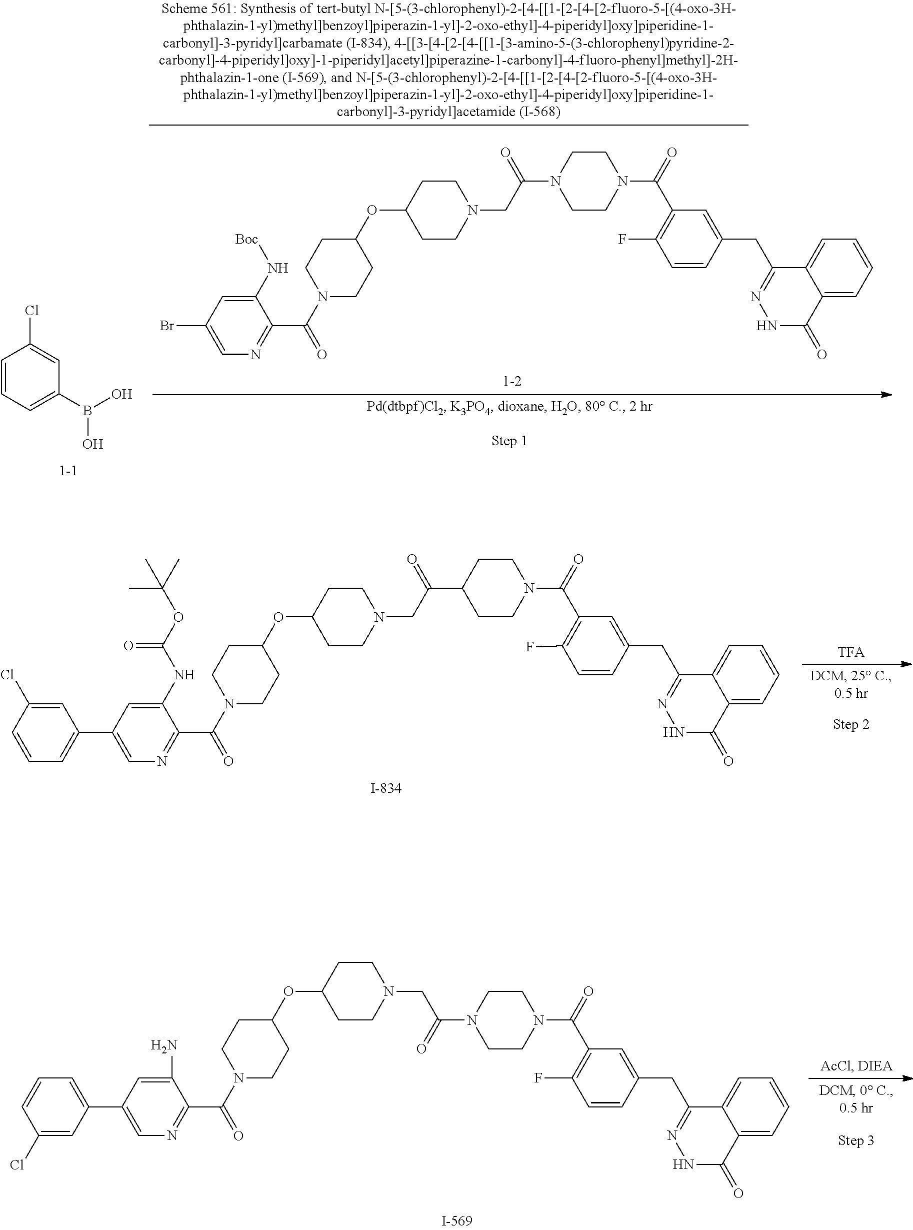 [SMILES: OB(O)c1cccc(Cl)c1 ; CC(C)(C)OC(=O)Nc1cc(Br)cnc1C(=O)N1CCC(OC2CCN(CC(=O)N3CCN(C(=O)c4cc(Cc5n[nH]c(=O)c6ccccc56)ccc4F)CC3)CC2)CC1 ; CC(C)(C)OC(=O)Nc1cc(-c2cccc(Cl)c2)cnc1C(=O)N1CCC(OC2CCN(CC(=O)C3CCN(C(=O)c4cc(Cc5n[nH]c(=O)c6ccccc56)ccc4F)CC3)CC2)CC1 ; Nc1cc(-c2cccc(Cl)c2)cnc1C(=O)N1CCC(OC2CCN(CC(=O)N3CCN(C(=O)c4cc(Cc5n[nH]c(=O)c6ccccc56)ccc4F)CC3)CC2)CC1]

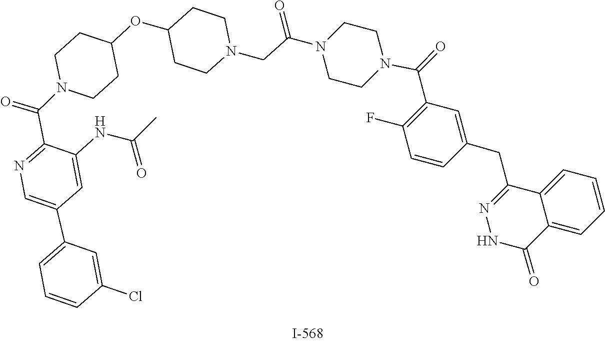 [SMILES: CC(=O)Nc1cc(-c2cccc(Cl)c2)cnc1C(=O)N1CCC(OC2CCN(CC(=O)N3CCN(C(=O)c4cc(Cc5n[nH]c(=O)c6ccccc56)ccc4F)CC3)CC2)CC1]

### Step 1: Synthesis of I-834

To a solution of Intermediate 1-2 (20 mg, 22.48 μmol, 1 eq) and Intermediate 1-1 (7.03 mg, 44.95 μmol, 2 eq) in dioxane (0.5 mL) and H₂O (0.1 mL) was added ditert-butyl(cyclopenta-1,4-dien-1-yl)phosphane;dichloropalladium;iron (1.46 mg, 2.25 μmol, 0.1 eq) and K₃PO₄ (14.31 mg, 67.43 μmol, 3 eq). The mixture was stirred at 80° C. for 1 hr. The mixture was diluted with water (5 mL) and extracted with DCM (5 mL*3). The combined organic layer was dried over Na₂SO₄, filtered and concentrated to give a residue. The residue was purified by prep-HPLC (column: CD01-Phenomenex luna C18 150*25*10 um; mobile phase: [water(TFA)-ACN]; gradient: 34%-64% B over 10 min). Then eluent was concentrated in vacuo and lyophilized. I-834 (6.74 mg, 6.34 μmol, 28.20% yield, 97.387% purity, TFA salt) was obtained as a white solid. LCMS (Method D): Rt=0.430 min, [M+H]⁺=921.2. ¹H NMR (400 MHz, METHANOL-d₄) δ=8.55 (d, J=1.6 Hz, 1H), 8.43-8.34 (m, 2H), 7.98-7.92 (m, 1H), 7.90-7.81 (m, 2H), 7.73 (s, 1H), 7.64 (d, J=7.6 Hz, 1H), 7.54-7.46 (m, 3H), 7.39-7.34 (m, 1H), 7.18 (t, J=9.6 Hz, 1H), 4.39 (s, 2H), 4.31 (s, 1H), 4.23 (s, 1H), 4.08-4.01 (m, 1H), 3.97 (s, 1H), 3.84-3.77 (m, 3H), 3.76-3.62 (m, 3H), 3.61-3.54 (m, 2H), 3.53-3.41 (m, 3H), 3.36 (s, 4H), 3.20-3.05 (m, 1H), 2.29-2.16 (m, 1H), 2.07 (s, 2H), 2.03 (s, 1H), 1.99-1.83 (m, 2H), 1.78-1.67 (m, 2H), 1.53 (s, 9H). ¹⁹F NMR (376 MHz, METHANOL-d₄) δ=−77.194, −120.749.

### Step 2: Synthesis of I-569

To a solution of I-834 (20 mg, 19.31 μmol, 1 eq, TFA alt) in DCM (0.5 mL) was added TFA (2.20 mg, 19.31 μmol, 1.43 μL, 1 eq). The mixture was stirred at 25° C. for 0.5 hr. The reaction mixture was concentrated in vacuo to give a residue. The residue was used directly without further purification. I-569 (7 mg, 7.32 μmol, 37.89% yield, 97.789% purity, TFA) was obtained as a yellow solid. LCMS (Method D): Rt=0.359 min, [M+H]⁺=821.2. ¹H NMR (400 MHz, METHANOL-d₄) δ=8.38-8.35 (m, 1H), 8.16 (s, 1H), 7.94 (d, J=5.6 Hz, 1H), 7.89-7.82 (m, 2H), 7.69 (s, 1H), 7.61-7.57 (m, 2H), 7.54-7.49 (m, 2H), 7.48 (d, J=2.4 Hz, 1H), 7.36-7.34 (m, 1H), 7.17 (t, J=9.6 Hz, 1H), 4.39 (s, 2H), 4.31 (s, 1H), 4.23 (s, 1H), 3.96 (s, 1H), 3.84-3.76 (m, 4H), 3.73 (d, J=4.4 Hz, 1H), 3.66 (d, J=11.6 Hz, 1H), 3.60-3.53 (m, 2H), 3.51 (s, 2H), 3.45 (s, 1H), 3.36 (s, 4H), 3.17-3.06 (m, 1H), 2.27-2.16 (m, 1H), 2.07 (s, 3H), 1.99-1.90 (m, 2H), 1.89-1.79 (m, 1H), 1.69 (d, J=4.0 Hz, 2H). ¹⁹F NMR (376 MHz, METHANOL-d₄) δ=−77.449, −120.749.

### Step 3: Synthesis of I-568

To a solution of I-569 (40 mg, 42.76 μmol, 1 eq, TFA salt) and DIEA (16.58 mg, 128.29 μmol, 22.35 μL, 3 eq) in DCM (0.5 mL) was added acetyl chloride (3.36 mg, 42.76 μmol, 3.04 μL, 1 eq). The mixture was stirred at 0° C. for 0.5 hr. The mixture was diluted with water (5 mL) and extracted with DCM (5 mL*3). The combined organic layer was dried over Na₂SO₄, filtered and concentrated in vacuo to give a residue. The residue was purified by prep-HPLC (column: CD07-Daisogel SP-100-8-ODS-PK 150*25*10 um;mobile phase: [water (NH₄HCO₃)-ACN]; gradient: 25%-55% B over 10 min). Then the eluent was concentrated in vacuo and lyophilized. I-568 (11.6 mg, 13.44 μmol, 31.42% yield, 100% purity) was obtained as a white solid. LCMS (Method D): Rt=0.395 min, [M+H]⁺=863.4. ¹H NMR (400 MHz, METHANOL-d₄) δ=8.64 (d, J=1.6 Hz, 1H), 8.39-8.35 (m, 1H), 8.30 (s, 1H), 7.97-7.94 (m, 1H), 7.90-7.82 (m, 2H), 7.74 (s, 1H), 7.64 (d, J=7.6 Hz, 1H), 7.53-7.46 (m, 3H), 7.38 (s, 1H), 7.16 (t, J=9.2 Hz, 1H), 4.39 (s, 2H), 4.07-4.00 (m, 1H), 3.80 (s, 2H), 3.73 (s, 2H), 3.66 (d, J=1.6 Hz, 2H), 3.58-3.48 (m, 5H), 3.26-3.19 (m, 2H), 2.83-2.72 (m, 2H), 2.36-2.22 (m, 2H), 2.17 (s, 3H), 1.89-1.87 (m, 5H), 1.74-1.54 (m, 5H). ¹⁹F NMR (376 MHz, METHANOL-d₄) δ=−120.802.

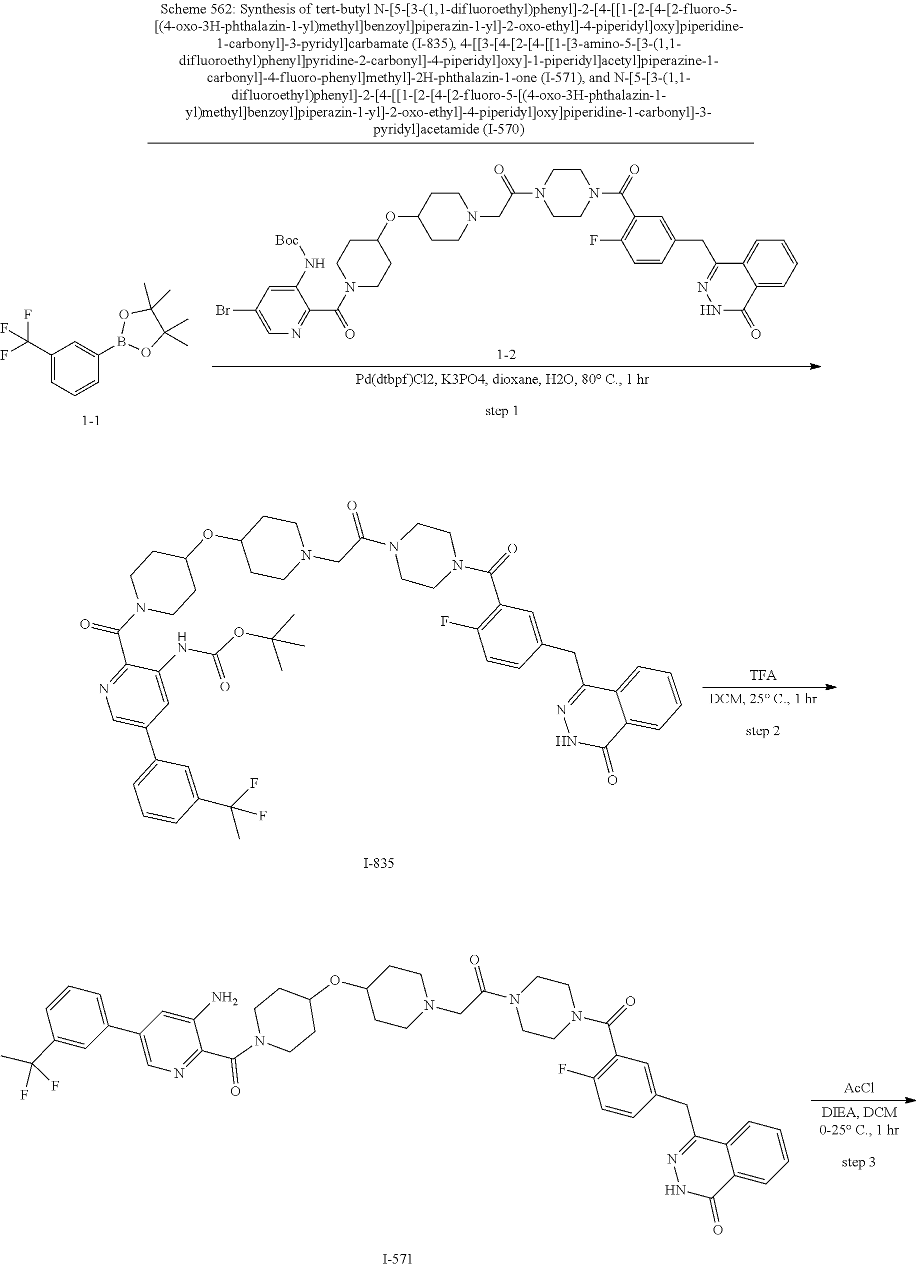

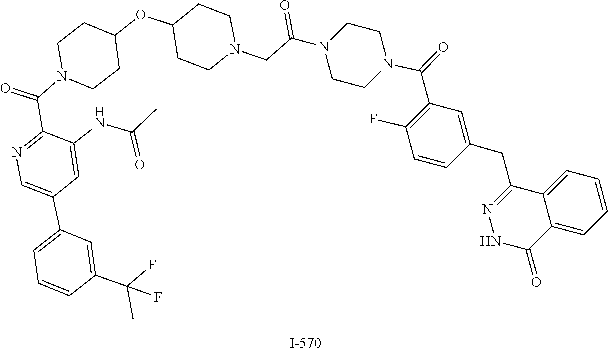 [SMILES: CC(=O)Nc1cc(-c2cccc(C(C)(F)F)c2)cnc1C(=O)N1CCC(OC2CCN(CC(=O)N3CCN(C(=O)c4cc(Cc5n[nH]c(=O)c6ccccc56)ccc4F)CC3)CC2)CC1]

### Step 1: Synthesis of I-835

A mixture of Intermediate 1-1 (20 mg, 74.60 μmol, 1 eq), Intermediate 1-2 (66.38 mg, 74.60 μmol, 1 eq), ditert-butyl(cyclopenta-1,4-dien-1-yl)phosphane;dichloropalladium;iron (4.86 mg, 7.46 μmol, 0.1 eq), and K₃PO₄ (31.67 mg, 149.19 μmol, 2 eq) in dioxane (0.4 mL) and H₂O (0.08 mL) was degassed and purged with N₂ 3 times, and then the mixture was stirred at 80° C. for 1 hr under N₂ atmosphere. The mixture was diluted with H₂O (5 mL) and extracted with EA (5 mL*3), then the combined organic layer was dried over Na₂SO₄, filtered and concentrated under reduced pressure to give a residue. The crude product was purified by reversed-phase chromatography (0.1% FA condition). The eluent was concentrated under reduced pressure to remove ACN, then lyophilized to afford product. I-835 (65 mg, 63.52 μmol, 85.15% yield, 97.438% purity, FA salt) was obtained as a white solid. LCMS (Method D): Rt=0.401 min, [M+H]⁺=951.3. ¹H NMR (400 MHz, METHANOL-d₄) δ=8.57 (d, J=2.0 Hz, 1H), 8.48 (br s, 1H), 8.44 (d, J=2.0 Hz, 1H), 8.39-8.34 (m, 1H), 7.99-7.92 (m, 1H), 7.91-7.76 (m, 4H), 7.66-7.59 (m, 2H), 7.50 (br d, J=2.4 Hz, 1H), 7.37 (br t, J=5.2 Hz, 1H), 7.17 (t, J=9.2 Hz, 1H), 4.39 (s, 2H), 4.13-3.97 (m, 1H), 3.86-3.63 (m, 7H), 3.62-3.40 (m, 6H), 3.33 (br s, 2H), 2.94 (br d, J=1.2 Hz, 2H), 2.67-2.41 (m, 2H), 2.05-1.87 (m, 7H), 1.69 (br s, 4H), 1.54 (s, 9H). ¹⁹F NMR: (376 MHz, METHANOL-d₄) δ=−88.843, 120.772.

### Step 2: Synthesis of I-571

A solution of I-835 (50 mg, 52.57 μmol, 1 eq) in DCM (0.4 mL) and TFA (0.2 mL) was stirred at 25° C. for 1 h. The reaction mixture was concentrated under reduced pressure then washed by PE (3*1 mL) to give a residue. The crude product was purified by reversed-phase chromatography (0.1% TFA condition). The eluent was concentrated under reduced pressure to remove ACN and then lyophilized to afford product. I-571 (23 mg, 23.73 μmol, 45.13% yield, 99.539% purity, TFA salt) was obtained as a yellow solid. LCMS (Method D): Rt=0.368 min, [M+H]⁺=851.2. ¹H NMR (400 MHz, METHANOL-d₄) δ=8.41-8.32 (m, 1H), 8.16 (br d, J=4.4 Hz, 1H), 7.98-7.91 (m, 1H), 7.90-7.80 (m, 2H), 7.80-7.70 (m, 2H), 7.64-7.48 (m, 4H), 7.38-7.34 (m, 1H), 7.17 (t, J=9.2 Hz, 1H), 4.39 (s, 2H), 4.32-4.19 (m, 2H), 3.96 (br s, 1H), 3.87-3.59 (m, 7H), 3.58-3.41 (m, 5H), 3.35 (br d, J=4.8 Hz, 4H), 3.16-3.05 (m, 1H), 2.30-2.14 (m, 1H), 2.06 (br s, 2H), 2.02-1.89 (m, 6H), 1.85-1.56 (m, 3H). ¹⁹F NMR (377 MHz, METHANOL-d₄) δ=−77.337, −88.897, −120.671.

### Step 3: Synthesis of I-570

To a solution of I-571 (70 mg, 72.54 μmol, 1 eq, TFA salt) in DCM (0.7 mL) was added DIEA (18.75 mg, 145.09 μmol, 25.27 μL, 2 eq), then acetyl chloride (5.69 mg, 72.54 μmol, 5.16 μL, 1 eq) was added in at 0° C. The mixture was stirred at 25° C. for 0.5 hr. The residue was diluted with H₂O (5 mL) and extracted with DCM (5 mL*3), then the combined organic layer was dried over Na₂SO₄, filtered and concentrated under reduced pressure to give a residue. The crude product was purified by reversed-phase chromatography (0.1% TFA condition). The eluent was concentrated under reduced pressure to remove ACN, then lyophilized to afford product. I-570 (23 mg, 22.20 μmol, 30.60% yield, 97.192% purity, TFA salt) was obtained as a white solid. LCMS (Method D): Rt=0.347 min, [M+H]⁺=893.3. ¹H NMR (400 MHz, METHANOL-d₄) δ=8.67 (s, 1H), 8.37 (d, J=8.0 Hz, 1H), 8.28 (s, 1H), 7.99-7.92 (m, 1H), 7.90-7.76 (m, 4H), 7.66-7.59 (m, 2H), 7.50 (br s, 1H), 7.37 (br t, J=6.8 Hz, 1H), 7.17 (t, J=9.2 Hz, 1H), 4.39 (s, 2H), 4.08-3.97 (m, 1H), 3.87-3.65 (m, 8H), 3.63-3.51 (m, 4H), 3.49-3.44 (m, 1H), 3.20-3.05 (m, 3H), 2.92-2.72 (m, 2H), 2.17 (s, 3H), 1.98 (br t, J=18.4 Hz, 8H), 1.70 (br d, J=2.4 Hz, 4H). ¹⁹F NMR: (376 MHz, METHANOL-d₄) δ=−76.939, −88.888, −120.779.

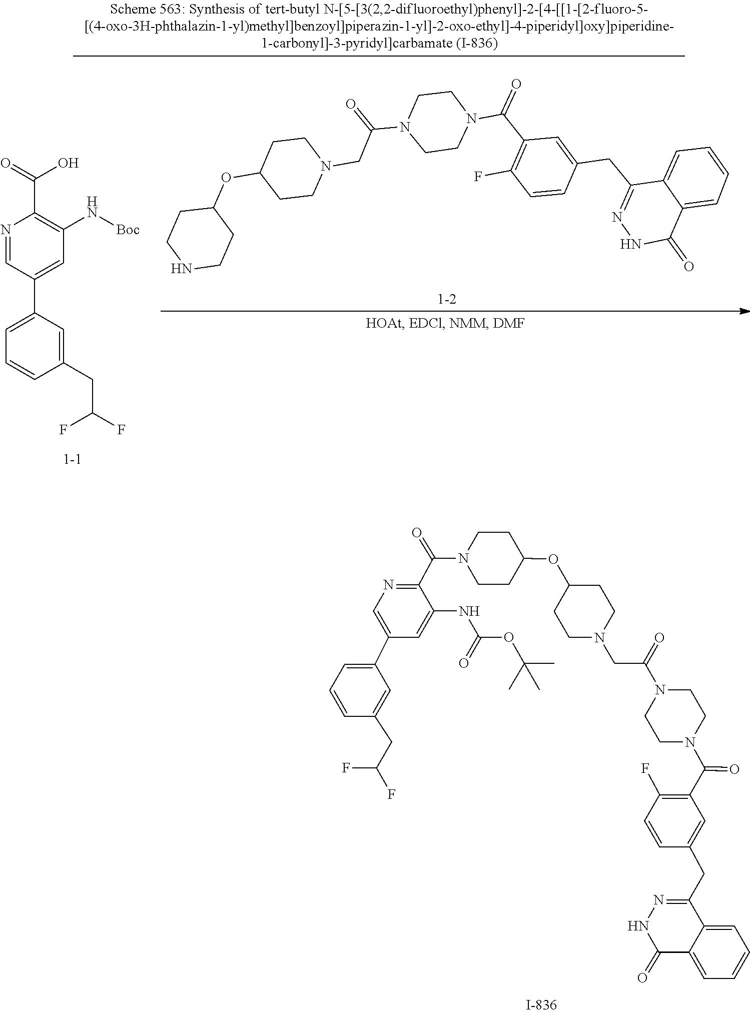 [SMILES: CC(C)(C)OC(=O)Nc1cc(-c2cccc(CC(F)F)c2)cnc1C(=O)O ; O=C(CN1CCC(OC2CCNCC2)CC1)N1CCN(C(=O)c2cc(Cc3n[nH]c(=O)c4ccccc34)ccc2F)CC1 ; CC(C)(C)OC(=O)Nc1cc(-c2cccc(CC(F)F)c2)cnc1C(=O)N1CCC(OC2CCN(CC(=O)N3CCN(C(=O)c4cc(Cc5n[nH]c(=O)c6ccccc56)ccc4F)CC3)CC2)CC1]

To a solution of intermediate 1-1 (30 mg, 79.29 μmol, 1 eq) and intermediate 1-2 (46.83 mg, 79.29 μmol, 1 eq) in DMF (1 mL) was added HOAt (10.79 mg, 79.29 μmol, 11.09 μL, 1 eq), EDCI (45.60 mg, 237.86 μmol, 3 eq) and NMM (40.10 mg, 396.44 μmol, 43.59 μL, 5 eq), and then the mixture was stirred at 40° C. for 1 hour. The reaction mixture was diluted with water (80 mL) and extracted with EA (30 mL*2). The combined organic layers were concentrated under reduced pressure to give a residue. The crude product was purified by reversed-phase chromatography (0.1% FA condition). The eluent was concentrated under reduced pressure to remove ACN and then lyophilized to give the product. I-836 (27.38 mg, 27.46 μmol, 34.63% yield, 100% purity, FA salt) was obtained as a yellow solid. LCMS (Method D): Rt: 0.423 min, [M+H]⁺=951.4. ¹H NMR (400 MHz, METHANOL-d₄) δ=8.56-8.50 (m, 1H), 8.42 (d, J=1.6 Hz, 1H), 8.37 (d, J=7.6 Hz, 1H), 7.98-7.91 (m, 1H), 7.90-7.80 (m, 2H), 7.68-7.56 (m, 2H), 7.52-7.47 (m, 2H), 7.43-7.33 (m, 2H), 7.19-7.14 (m, 1H), 6.28-5.89 (m, 1H), 4.39 (s, 2H), 4.12-4.00 (m, 1H), 3.84-3.73 (m, 3H), 3.68 (s, 3H), 3.63-3.51 (m, 4H), 3.44 (s, 1H), 3.40-3.32 (m, 5H), 3.28-3.19 (m, 2H), 2.95-2.82 (m, 2H), 2.56-2.34 (m, 2H), 2.02-1.87 (m, 4H), 1.75-1.61 (m, 4H), 1.53 (s, 9H). ¹⁹F NMR (376 MHz, METHANOL-d₄) δ=−116.966, −120.787.

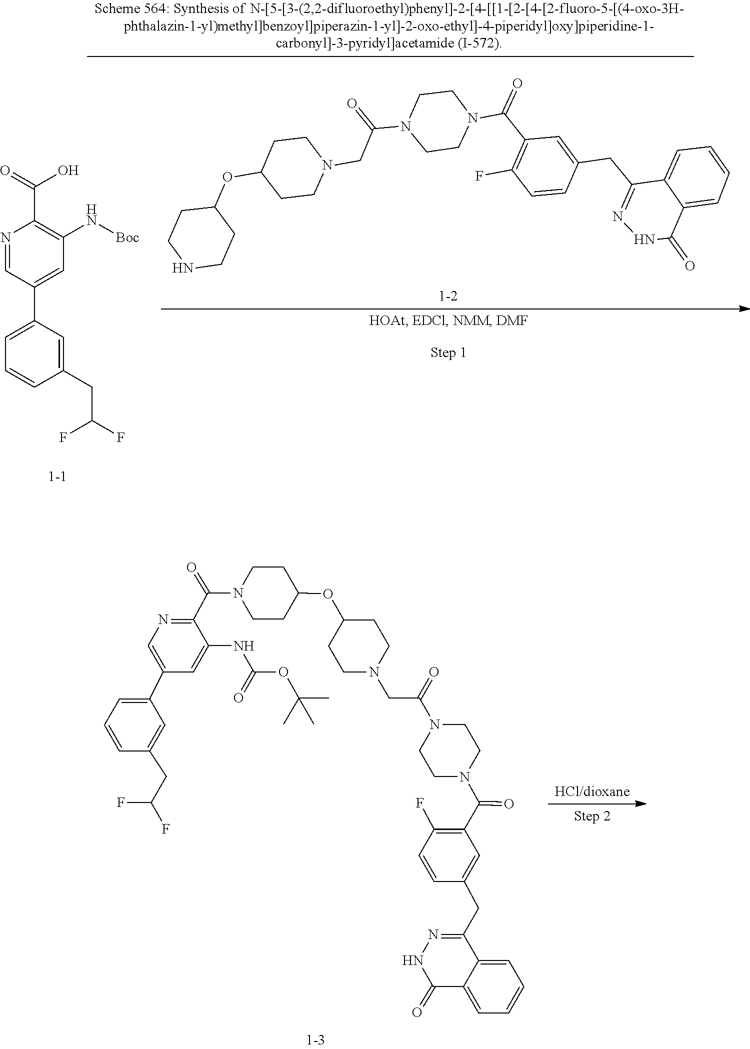 [SMILES: CC(C)(C)OC(=O)Nc1cc(-c2cccc(CC(F)F)c2)cnc1C(=O)O ; O=C(CN1CCC(OC2CCNCC2)CC1)N1CCN(C(=O)c2cc(Cc3n[nH]c(=O)c4ccccc34)ccc2F)CC1 ; CC(C)(C)OC(=O)Nc1cc(-c2cccc(CC(F)F)c2)cnc1C(=O)N1CCC(OC2CCN(CC(=O)N3CCN(C(=O)c4cc(Cc5n[nH]c(=O)c6ccccc56)ccc4F)CC3)CC2)CC1]

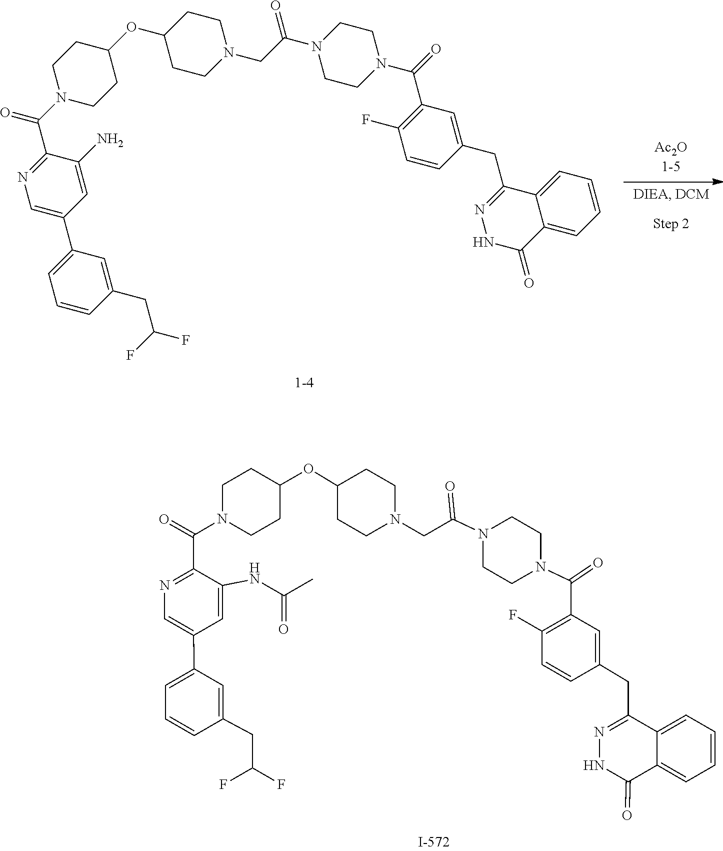 [SMILES: Nc1cc(-c2cccc(CC(F)F)c2)cnc1C(=O)N1CCC(OC2CCN(CC(=O)N3CCN(C(=O)c4cc(Cc5n[nH]c(=O)c6ccccc56)ccc4F)CC3)CC2)CC1 ; CC(=O)Nc1cc(-c2cccc(CC(F)F)c2)cnc1C(=O)N1CCC(OC2CCN(CC(=O)N3CCN(C(=O)c4cc(Cc5n[nH]c(=O)c6ccccc56)ccc4F)CC3)CC2)CC1]

### Step 1: Synthesis of Intermediate 1-3

To a solution of intermediate 1-1 (70 mg, 185.00 μmol, 1 eq) and intermediate 1-2 (109.28 mg, 185.00 μmol, 1 eq) in DMF (2 mL) was added HOAt (25.18 mg, 185.00 μmol, 25.88 μL, 1 eq), EDCI (106.40 mg, 555.01 μmol, 3 eq) and NMM (93.56 mg, 925.02 μmol, 101.70 μL, 5 eq), then the mixture was stirred at 40° C. for 1 hour. The reaction mixture was diluted with H₂O (80 mL) and extracted with EA (30 mL*2). The combined organic layers were concentrated under reduced pressure to give Intermediate 1-3 (150 mg, 157.72 μmol, 85.25% yield) as a yellow solid. LCMS (Method D): Rt=0.414 min, [M+H]⁺=951.2.

### Step 2: Synthesis of Intermediate 1-4

To a solution of Intermediate 1-3 (150 mg, 157.72 μmol, 1 eq) in DCM (2 mL) was added HCl/dioxane (2 M, 394.30 μL, 5 eq), then the mixture was stirred at 20° C. for 1 hour. The reaction mixture was concentrated under reduced pressure to give Intermediate 1-4 (100 mg, 112.69 μmol, 71.45% yield, HCl salt) as a yellow solid. LCMS (Method D): Rt=0.356 min, [M+H]⁺=851.2.

### Step 3: Synthesis of I-572

To a solution of intermediate 1-4 (90 mg, 101.42 μmol, 1 eq, HCl salt) in DCM (1 mL) was added TEA (30.79 mg, 304.26 μmol, 42.35 μL, 3 eq) and intermediate 1-5 (7.96 mg, 101.42 μmol, 7.21 μL, 1 eq) at 0° C., then the mixture was stirred at 0° C. for 1 hour. The reaction mixture was diluted with water (80 mL) and extracted with DCM (30 mL*2). The combined organic layers were concentrated under reduced pressure to give a residue. The crude product was purified by reversed-phase HPLC (neutral condition). The eluent was concentrated under reduced pressure to remove ACN and then lyophilized to give I-572 (18.2 mg, 20.38 μmol, 20.10% yield, 100% purity) as a yellow solid. LCMS (Method D): Rt=0.360 min, [M+H]⁺=893.3 ¹H NMR (400 MHz, METHANOL-d₄) δ=8.64 (d, J=1.6 Hz, 1H), 8.37 (d, J=6.8 Hz, 1H), 8.32-8.23 (m, 1H), 8.00-7.92 (m, 1H), 7.90-7.81 (m, 2H), 7.63 (d, J=4.0 Hz, 2H), 7.52-7.46 (m, 2H), 7.40 (d, J=8.0 Hz, 2H), 7.19-7.14 (m, 1H), 6.24-5.93 (m, 1H), 4.39 (s, 2H), 4.08-4.00 (m, 1H), 3.82-3.46 (m, 12H), 3.26 (d, J=4.0 Hz, 3H), 3.23-3.18 (m, 2H), 2.77 (d, J=2.8 Hz, 2H), 2.31-2.21 (m, 2H), 2.17 (s, 3H), 1.97-1.84 (m, 4H), 1.71-1.56 (m, 4H). ¹⁹F NMR (376 MHz, METHANOL-d₄) δ=−116.996, −120.757.

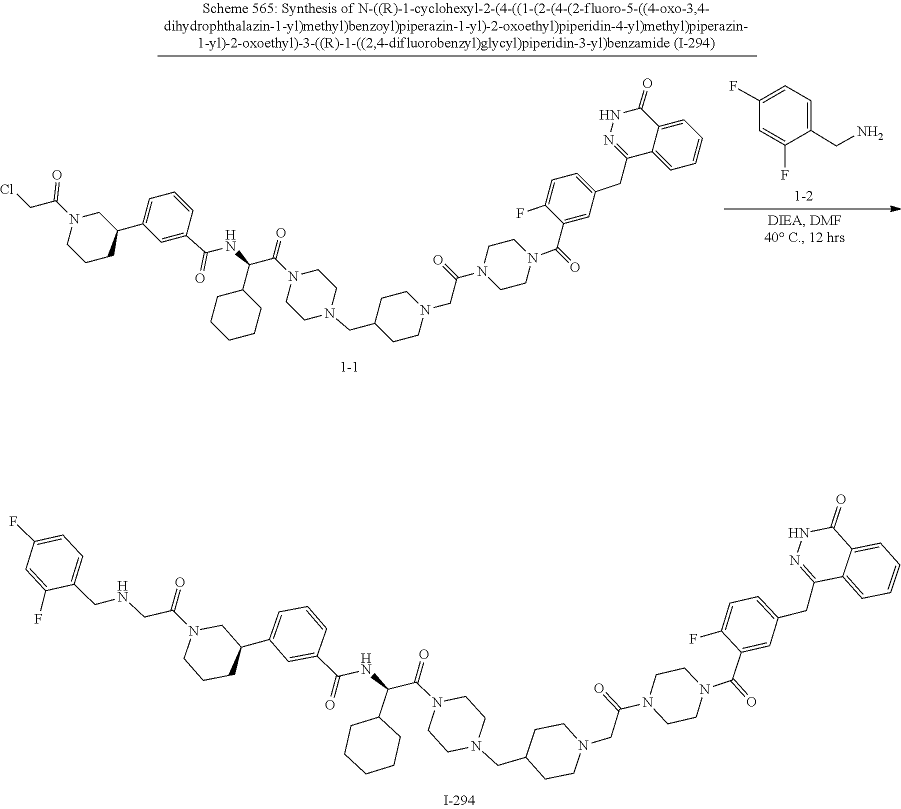 [SMILES: O=C(N[C@@H](C(=O)N1CCN(CC2CCN(CC(=O)N3CCN(C(=O)c4cc(Cc5n[nH]c(=O)c6ccccc56)ccc4F)CC3)CC2)CC1)C1CCCCC1)c1cccc([C@H]2CCCN(C(=O)CCl)C2)c1 ; NCc1ccc(F)cc1F ; O=C(N[C@@H](C(=O)N1CCN(CC2CCN(CC(=O)N3CCN(C(=O)c4cc(Cc5n[nH]c(=O)c6ccccc56)ccc4F)CC3)CC2)CC1)C1CCCCC1)c1cccc([C@H]2CCCN(C(=O)CNCc3ccc(F)cc3F)C2)c1]

To a solution of Intermediate 1-1 (600 mg, 604.46 μmol, 1 eq) in DMF (6 mL) was added Intermediate 1-2 (103.82 mg, 725.36 μmol, 1.2 eq) and DIPEA (234.37 mg, 1.81 mmol, 315.86 μL, 3 eq). The mixture was stirred at 40° C. for 12 hrs. The mixture was filtered. The filtrate was purified by reversed-phase HPLC (0.1% FA condition). The eluent was concentrated under reduced pressure to remove ACN and then lyophilized to give I-294 (191.09 mg, 172.44 μmol, 28.53% yield, 99.2% purity) as a white solid. LCMS (Method D): Rt: 0.317 min, [M+H]⁺=1099.2. ¹H NMR (400 MHz, METHANOL-d₄) δ=8.39-8.35 (m, 1H), 7.98-7.92 (m, 1H), 7.91-7.81 (m, 2H), 7.76 (s, 1H), 7.73-7.68 (m, 1H), 7.52-7.41 (m, 4H), 7.38 (s, 1H), 7.20-7.12 (m, 1H), 7.00-6.88 (m, 2H), 4.93-4.90 (m, 1H), 4.59-4.51 (m, 1H), 4.39 (s, 2H), 3.87 (s, 1H), 3.85-3.78 (m, 4H), 3.76-3.70 (m, 4H), 3.69-3.64 (m, 1H), 3.60-3.49 (m, 5H), 3.49-3.41 (m, 1H), 3.25 (s, 1H), 3.19 (s, 1H), 3.17-3.08 (m, 1H), 2.95-2.85 (m, 2H), 2.84-2.66 (m, 2H), 2.54-2.41 (m, 3H), 2.41-2.33 (m, 1H), 2.25-2.17 (m, 2H), 2.11-2.01 (m, 3H), 1.92-1.82 (m, 4H), 1.81-1.73 (m, 4H), 1.72-1.66 (m, 2H), 1.63-1.51 (m, 2H), 1.35-1.17 (m, 6H), 1.16-1.07 (m, 2H). ¹⁹F NMR (376 MHz, METHANOL-d₄) δ=−113.431, −116.283, −120.801. SFC: Rt: 5.742 min.

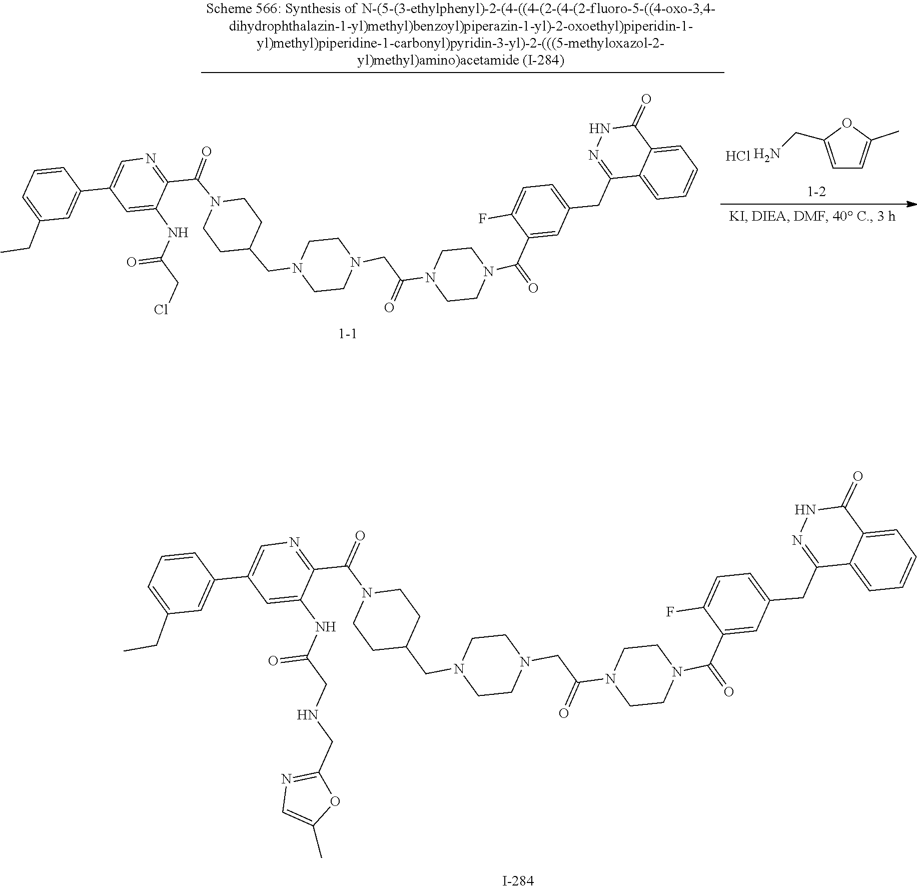

To a solution of Intermediate 1-1 (100 mg, 112.30 μmol, 1 eq) in DMF (2 mL) was added DIEA (58.06 mg, 449.22 μmol, 78.25 μL, 4 eq), KI (74.57 mg, 449.22 μmol, 4 eq) and Intermediate 1-2 (25.03 mg, 168.46 μmol, 1.5 eq, HCl salt). The mixture was stirred at 40° C. for 3 hrs. The mixture was filtered. The filtrate was purified by Prep-HPLC (column: Phenomenex luna C18 150*25 mm*10 um; mobile phase: [water (FA)-ACN]; gradient: 10%-40% B over 1 min) to afford I-284 (55.79 mg, 54.49 μmol, 48.52% yield, 98.862% purity, FA salt) as an off-white solid. LCMS (Method G): Rt=0.613 min, [M+H]⁺=966.6. ¹H NMR (400 MHz, METHANOL-d₄) δ=8.59 (m, 2H), 8.33 (m, 1H), 8.28 (s, 1H), 7.94-7.76 (m, 3H), 7.51-7.43 (m, 3H), 7.41-7.26 (m, 3H), 7.13 (m, 1H), 6.70 (s, 1H), 4.63 (m, 1H), 4.35 (s, 2H), 3.90 (s, 2H), 3.82-3.70 (m, 3H), 3.68-3.60 (m, 2H), 3.56-3.43 (m, 6H), 3.28 (br s, 3H), 3.19-3.10 (m, 1H), 2.98-2.78 (m, 8H), 2.76-2.63 (m, 4H), 2.26 (s, 3H), 2.09-1.96 (m, 1H), 1.95-1.86 (m, 1H), 1.74 (m, 1H), 1.39-1.24 (m, 5H).

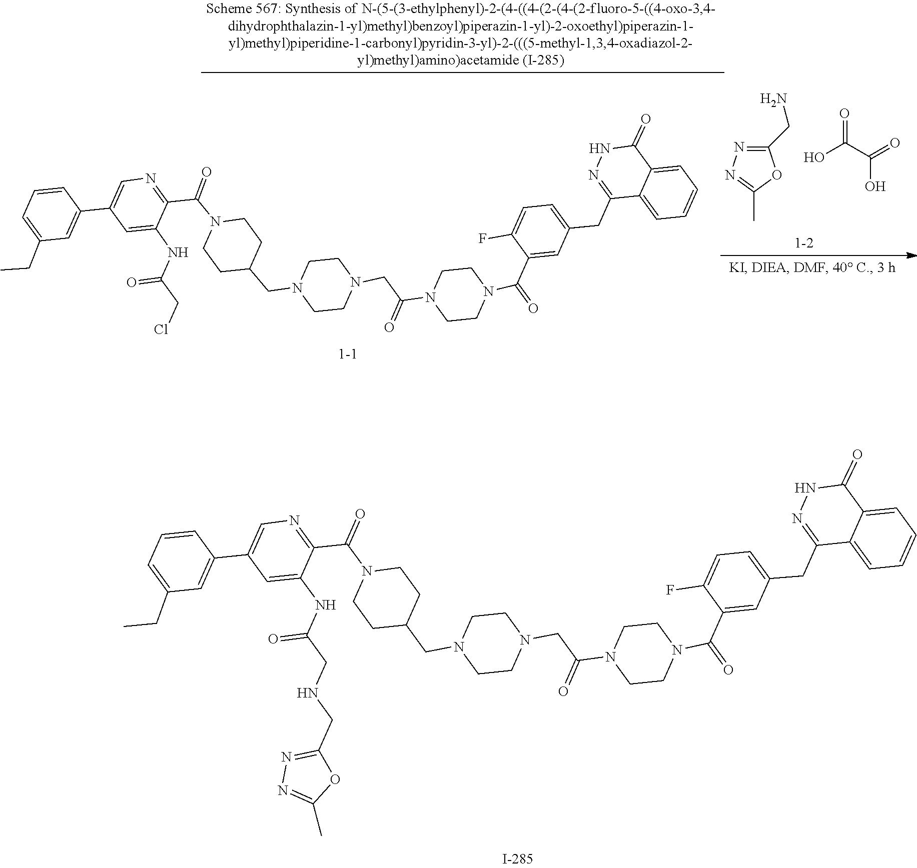

To a solution of Intermediate 1-1 (100 mg, 112.30 μmol, 1 eq) in DMF (2 mL) was added DIEA (58.06 mg, 449.22 μmol, 78.25 μL, 4 eq), KI (74.57 mg, 449.22 μmol, 4 eq) and Intermediate 1-2 (34.22 mg, 168.46 μmol, 1.5 eq, oxalic acid salt). The mixture was stirred at 40° C. for 3 h. The mixture was filtered. The filtrate was purified by Prep-HPLC (column: Waters Xbridge 150*25 mm*5 um; mobile phase: [water (NH₄HCO₃)-ACN]; gradient: 20%-50% B over 15 min) to afford I-285 (34.43 mg, 31.59 μmol, 28.13% yield, 99.183% purity, TFA salt) as an off-white solid. LCMS (Method G): Rt=0.581 min, [M+H]⁺=967.5. ¹H NMR (400 MHz, METHANOL-d₄) δ=8.71 (s, 1H), 8.59 (d, J=2.0 Hz, 1H), 8.35 (m, 1H), 7.97-7.91 (m, 1H), 7.88-7.79 (m, 2H), 7.53-7.45 (m, 3H), 7.43-7.35 (m, 2H), 7.30 (d, J=7.6 Hz, 1H), 4.62 (m, 1H), 4.37 (s, 2H), 4.08 (s, 2H), 3.87-3.66 (m, 5H), 3.59-3.49 (m, 4H), 3.35-3.31 (m, 2H), 3.27 (br s, 2H), 3.21-3.10 (m, 2H), 2.90 (m, 1H), 2.76-2.71 (m, 2H), 2.62-2.41 (m, 10H), 2.25 (m, 2H), 1.93-1.69 (m, 3H), 1.35-1.18 (m, 6H).

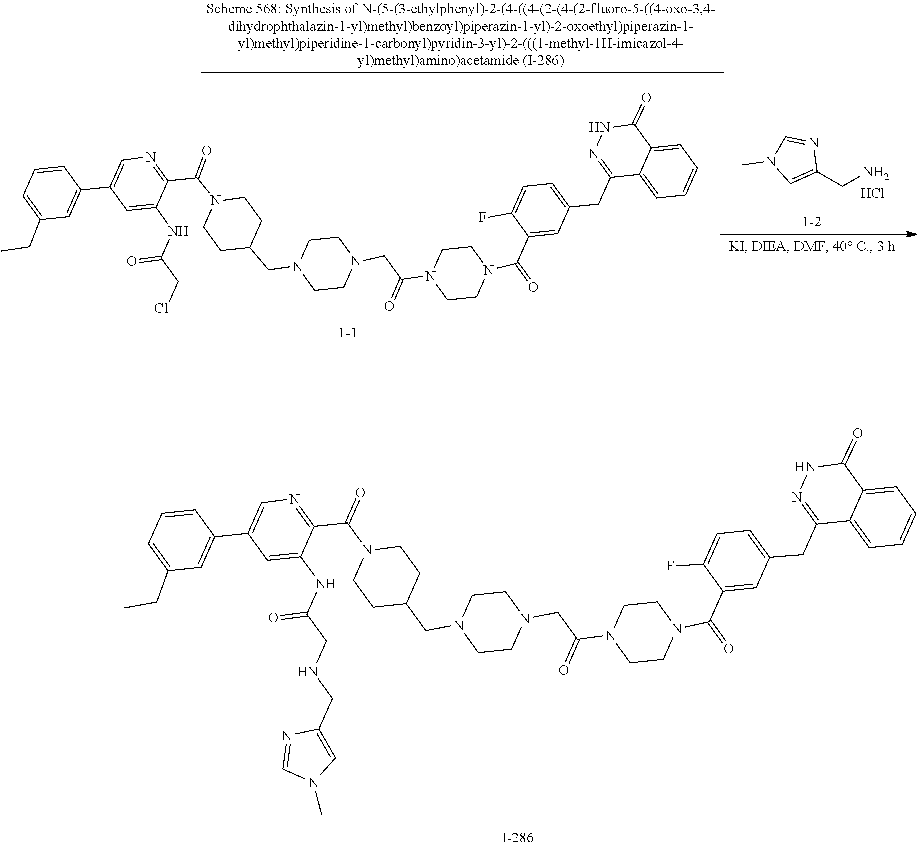

To a solution of Intermediate 1-1 (50 mg, 56.15 μmol, 1 eq) in DMF (1 mL) was added DIEA (29.03 mg, 224.61 μmol, 39.12 μL, 4 eq), KI (37.29 mg, 224.61 μmol, 4 eq) and Intermediate 1-2 (12.43 mg, 84.23 μmol, 1.5 e, HCl salt). The mixture was stirred at 40° C. for 3 hrs. The mixture was filtered. The filtrate was purified by Prep-HPLC (column: Waters Xbridge 150*25 mm*5 um; mobile phase: [water (NH₄HCO₃)-ACN]; gradient: 20%-50% B over 15 min) to afford I-286 (21.91 mg, 19.80 μmol, 17.63% yield, 97.521% purity, TFA salt) as an off-white solid. LCMS (Method G): Rt=0.588 min, [M+H]⁺=965.5. ¹H NMR (400 MHz, METHANOL-d₄) δ=8.93 (s, 1H), 8.75 (d, J=2.0 Hz, 1H), 8.51 (m, 1H), 8.13-8.06 (m, 1H), 8.05-7.94 (m, 2H), 7.73-7.61 (m, 4H), 7.60-7.50 (m, 2H), 7.46 (d, J=7.6 Hz, 1H), 7.35-7.26 (m, 2H), 4.79 (m, 1H), 4.53 (s, 2H), 4.01-3.78 (m, 9H), 3.73-3.63 (m, 2H), 3.57 (s, 2H), 3.52-3.47 (m, 2H), 3.46-3.39 (m, 2H), 3.38-3.25 (m, 2H), 3.03 (m, 1H), 2.89 (m, 2H), 2.75-2.54 (m, 7H), 2.36 (br dd, J=6.8, 13.1 Hz, 2H), 2.07-1.87 (m, 3H), 1.53-1.22 (m, 6H).

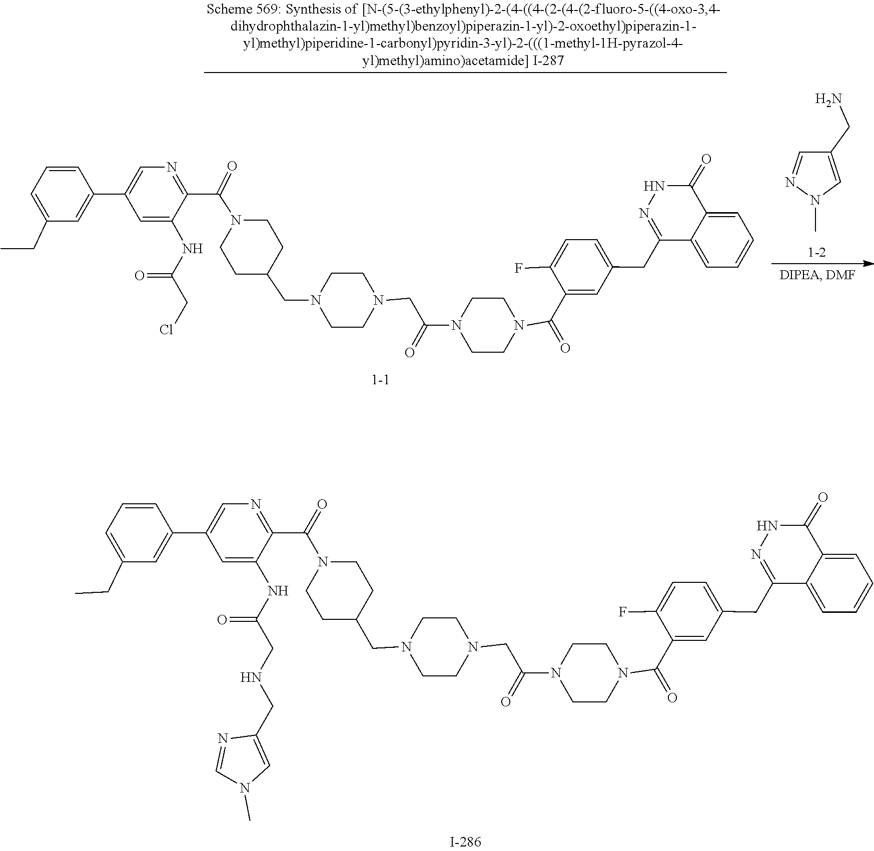

To a solution of Intermediate 1-1 (100 mg, 112.30 μmol, 1 eq) and Intermediate 1-2 (24.96 mg, 224.61 μmol, 2 eq) in DMF (1 mL) was added DIEA (43.54 mg, 336.91 μmol, 58.68 μL, 3 eq). The mixture was stirred at 40° C. for 2 hr. The reaction mixture was diluted with H₂O (2 mL) and extracted with EA (5 mL*3). The combined organic layers were washed with brine (5 mL*3), dried over Na₂SO₄, filtered and concentrated under reduced pressure to give a residue. The residue was purified by prep-HPLC (column: Welch Xtimate C18 150*30 mm*5 um; mobile phase: [water (FA)-ACN]; gradient: 9%-39% B over 10 min), and the eluent was concentrated and lyophilized to give a product. I-287 (32.55 mg, 30.82 μmol, 27.44% yield, 95.730% purity, FA salt) was obtained as a white solid. LCMS (Method D): Retention time: 0.331 min, [M+H]⁺=965.4. ¹H NMR (400 MHz, METHANOL-d₄) δ=8.61 (d, J=2.8 Hz, 2H), 8.45 (d, J=16.4 Hz, 1H), 8.36 (d, J=7.2 Hz, 1H), 7.97-7.91 (m, 1H), 7.89-7.79 (m, 2H), 7.71 (s, 1H), 7.59 (s, 1H), 7.54-7.46 (m, 3H), 7.45-7.39 (m, 1H), 7.38-7.27 (m, 2H), 7.19-7.09 (m, 1H), 4.67 (d, J=12.4 Hz, 1H), 4.37 (s, 2H), 3.93-3.83 (m, 5H), 3.82-3.72 (m, 3H), 3.70-3.59 (m, 4H), 3.56-3.32 (m, 6H), 3.21-3.11 (m, 1H), 2.95-2.79 (m, 4H), 2.79-2.66 (m, 7H), 2.58-2.47 (m, 2H), 2.05-1.88 (m, 2H), 1.75 (d, J=12.4 Hz, 1H), 1.42-1.33 (m, 1H), 1.32-1.22 (m, 4H). ¹⁹FNMR (376 MHz, METHANOL-d₄) δ=−120.67.

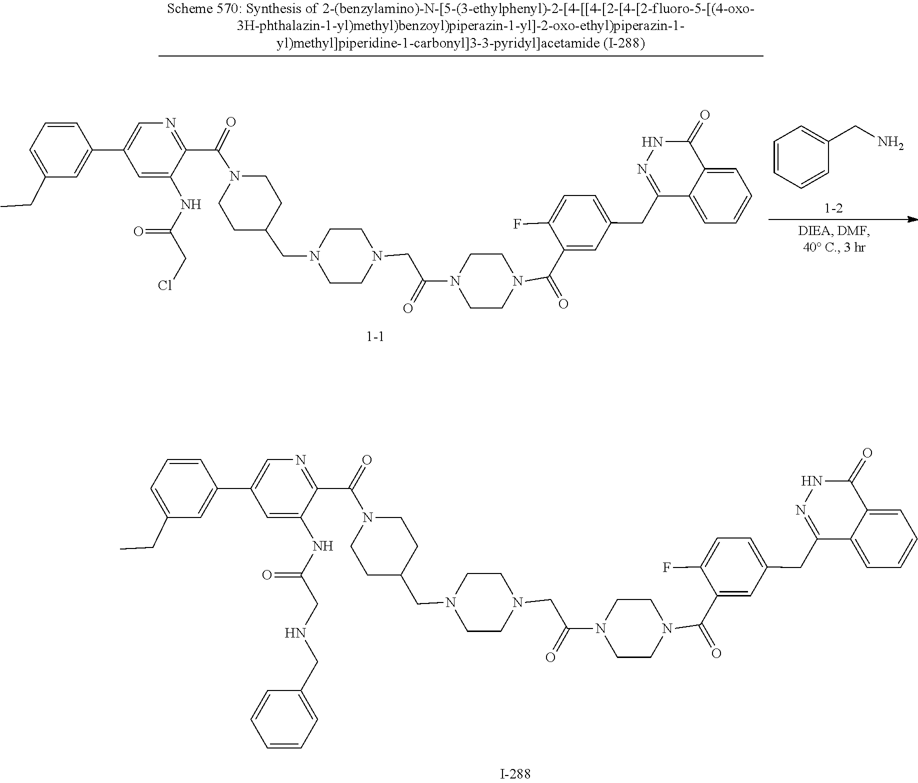

To a solution of Intermediate 1-1 (100 mg, 112.30 μmol, 1 eq) and Intermediate 1-2 (24.07 mg, 224.61 μmol, 24.48 μL, 2 eq) in DMF (1 mL) was added DIEA (43.54 mg, 336.91 μmol, 58.68 μL, 3 eq). The mixture was stirred at 40° C. for 3 hr. The reaction mixture was concentrated in vacuum. The residue was purified by prep-HPLC (column: Phenomenex luna C18 150*25 mm*10 um; mobile phase: [water (FA)-ACN]; gradient: 7%-37% B over 8 min) and the eluent was concentrated under reduced pressure to remove ACN and lyophilized to give product. I-288 (35.18 mg, 33.48 μmol, 29.81% yield, 95.852% purity, FA salt) was obtained as a white solid. LCMS (Method D): Rt=0.349 min, [M+H]⁺=961.7. ¹H NMR (400 MHz, METHANOL-d₄) δ=8.70 (s, 1H), 8.61 (d, J=1.6 Hz, 1H), 8.42 (s, 1H), 8.37 (d, J=7.6 Hz, 1H), 7.94 (d, J=6.4 Hz, 1H), 7.90-7.81 (m, 2H), 7.54-7.43 (m, 6H), 7.38-7.27 (m, 5H), 7.17-7.14 (m, 1H), 4.67 (d, J=11.6 Hz, 1H), 4.38 (s, 2H), 3.89 (s, 2H), 3.87-3.61 (m, 6H), 3.54-3.47 (m, 4H), 3.39 (s, 3H), 3.19-3.11 (m, 1H), 2.87-2.84 (m, 1H), 2.78-2.71 (m, 4H), 2.68 (s, 6H), 2.46-2.34 (m, 2H), 1.96-1.83 (m, 2H), 1.75 (d, J=10.8 Hz, 1H), 1.29-1.27 (m, 4H), 1.26-1.17 (m, 1H). ¹⁹F NMR (376 MHz, METHANOL-d₄) δ=−120.800.

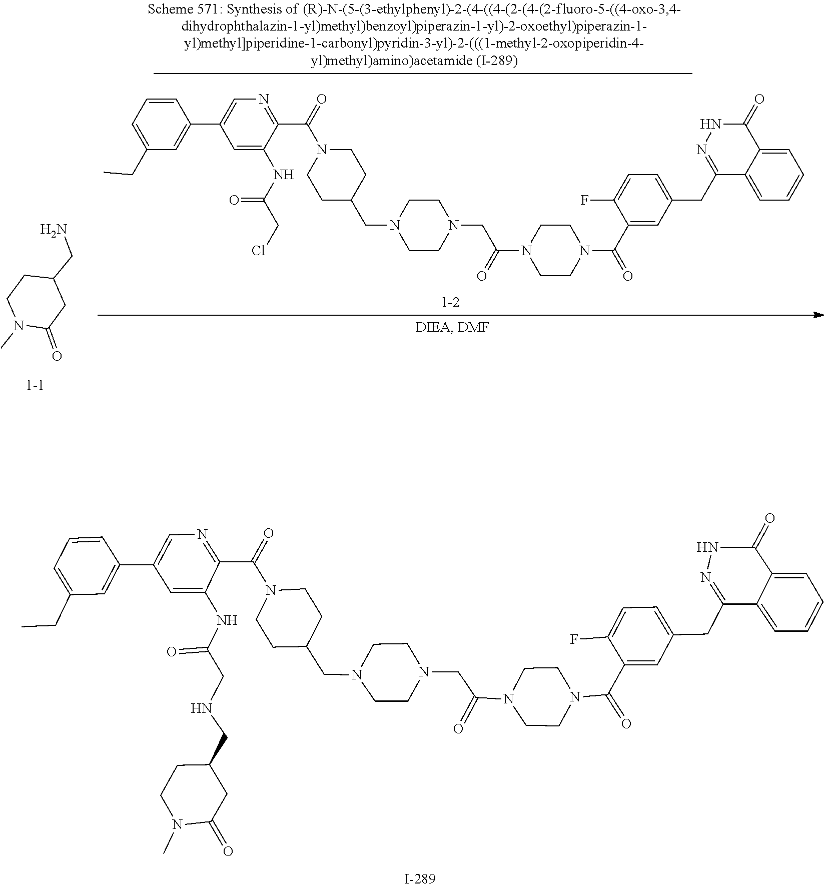

A solution of Intermediate 1-2 (200 mg, 224.61 μmol, 1 eq) and Intermediate 1-1 (48.15 mg, 269.53 μmol, 1.2 eq) in DMF (2 mL) was added DIEA (134.44 mg, 1.04 mmol, 181.18 μL, 4.63 eq). The reaction mixture was stirred and heated at 40° C. for 2 h. The reaction was diluted with DCM, washed with water (2 mL) and extracted with DCM (2 mL*3), the combined organic phase was dried by Na₂SO₄, and concentrated under reduced pressure to give a residue. The crude was purified by prep-HPLC (column: DAICEL CHIRALPAK IK (250 mm*30 mm, 10 um); mobile phase: [Hexane-IPA]; B %: 100%, isocratic elution mode) and the eluent was concentrated to remove ACN and lyophilized to give product. I-289 (17 mg, 16.30 μmol, 14.12% yield, 95.512% purity) was obtained as a yellow solid. LCMS (Method D): Retention time: 0.328 min, [M+H]⁺=996.4. SFC: Retention time: 3.948 min. ¹H NMR (400 MHz, METHANOL-d₄) δ=8.68 (s, 1H), 8.37 (d, J=6.0 Hz, 2H), 8.00-7.92 (m, 1H), 7.92-7.81 (m, 2H), 7.55-7.47 (m, 3H), 7.44 (t, J=7.6 Hz, 1H), 7.41-7.31 (m, 2H), 7.17 (t, J=8.8 Hz, 1H), 4.66 (d, J=12.0 Hz, 1H), 4.39 (s, 2H), 4.07 (s, 2H), 3.93-3.74 (m, 4H), 3.73-3.59 (m, 3H), 3.53 (s, 1H), 3.50-3.41 (m, 4H), 3.36 (d, J=4.4 Hz, 2H), 3.29-3.18 (m, 3H), 3.15-3.04 (m, 4H), 3.02-2.89 (m, 6H), 2.80-2.69 (m, 3H), 2.64-2.56 (m, 1H), 2.41-2.29 (m, 1H), 2.25-2.05 (m, 3H), 1.98 (d, J=12.4 Hz, 1H), 1.82-1.74 (m, 1H), 1.74-1.63 (m, 1H), 1.61-1.51 (m, 1H), 1.50-1.41 (m, 1H), 1.40-1.36 (m, 1H), 1.29 (t, J=7.6 Hz, 4H). ¹⁹F NMR (377 MHz, METHANOL-d₄) δ=−120.802.

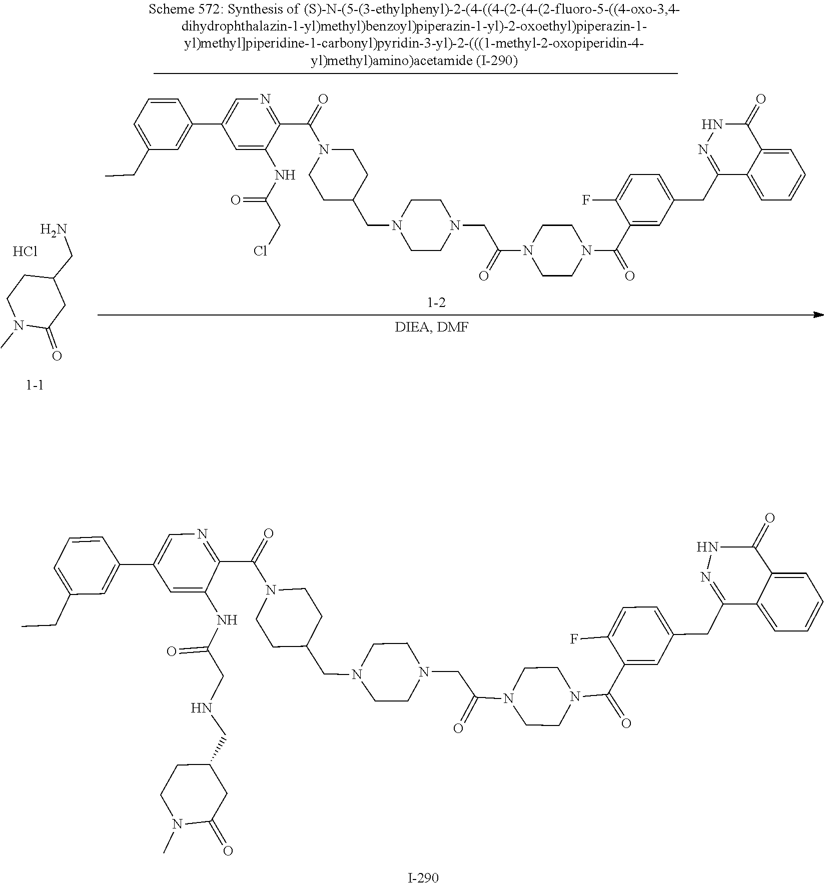

A solution of Intermediate 1-2 (200 mg, 224.61 μmol, 1 eq) and Intermediate 1-1 (48.15 mg, 269.53 μmol, 1.2 eq) in DMF (2 mL) was added DIEA (134.44 mg, 1.04 mmol, 181.18 μL, 4.63 eq). The reaction mixture was stirred and refluxed at 40° C. for 2 h. The reaction mixture was diluted with DCM, washed with water (2 mL) and extracted with DCM (2 mL*3), the combined organic phase was dried by Na₂SO₄, concentrated under reduced pressure to give a residue. The crude was purified by prep-HPLC (column: DAICEL CHIRALPAK IK (250 mm*30 mm, 10 um); mobile phase: [Hexane-IPA]; B %: 100%, isocratic elution mode), the eluent was concentrated to remove ACN and lyophilized to give product. I-290 (12 mg, 11.60 μmol, 10.05% yield, 96.335% purity) was obtained as a white solid. LCMS (Method H): Retention time: 0.370 min, [M+H]⁺=996.5. SFC: Retention time: 4.799 min. ¹H NMR (400 MHz, METHANOL-d₄) δ=8.85 (d, J=4.4 Hz, 1H), 8.59 (s, 1H), 8.37 (d, J=7.6 Hz, 1H), 7.94 (d, J=4.4 Hz, 1H), 7.91-7.79 (m, 2H), 7.56-7.46 (m, 3H), 7.45-7.35 (m, 2H), 7.31 (d, J=7.6 Hz, 1H), 7.16 (t, J=8.8 Hz, 1H), 4.68 (d, J=12.8 Hz, 1H), 4.62-4.52 (m, 1H), 4.38 (s, 2H), 3.82-3.70 (m, 4H), 3.67 (d, J=1.0 Hz, 1H), 3.56 (s, 1H), 3.51 (s, 1H), 3.46-3.38 (m, 4H), 3.27 (s, 2H), 3.23-3.10 (m, 2H), 2.97-2.87 (m, 4H), 2.78-2.70 (m, 2H), 2.65-2.59 (m, 2H), 2.58-2.42 (m, 8H), 2.29-2.25 (m, 2H), 2.17-2.06 (m, 3H), 1.99-1.86 (m, 2H), 1.81-1.71 (m, 1H), 1.61 (d, J=8.8 Hz, 1H), 1.29 (t, J=7.6 Hz, 6H). ¹⁹F NMR (377 MHz, METHANOL-d₄) δ=−120.802.

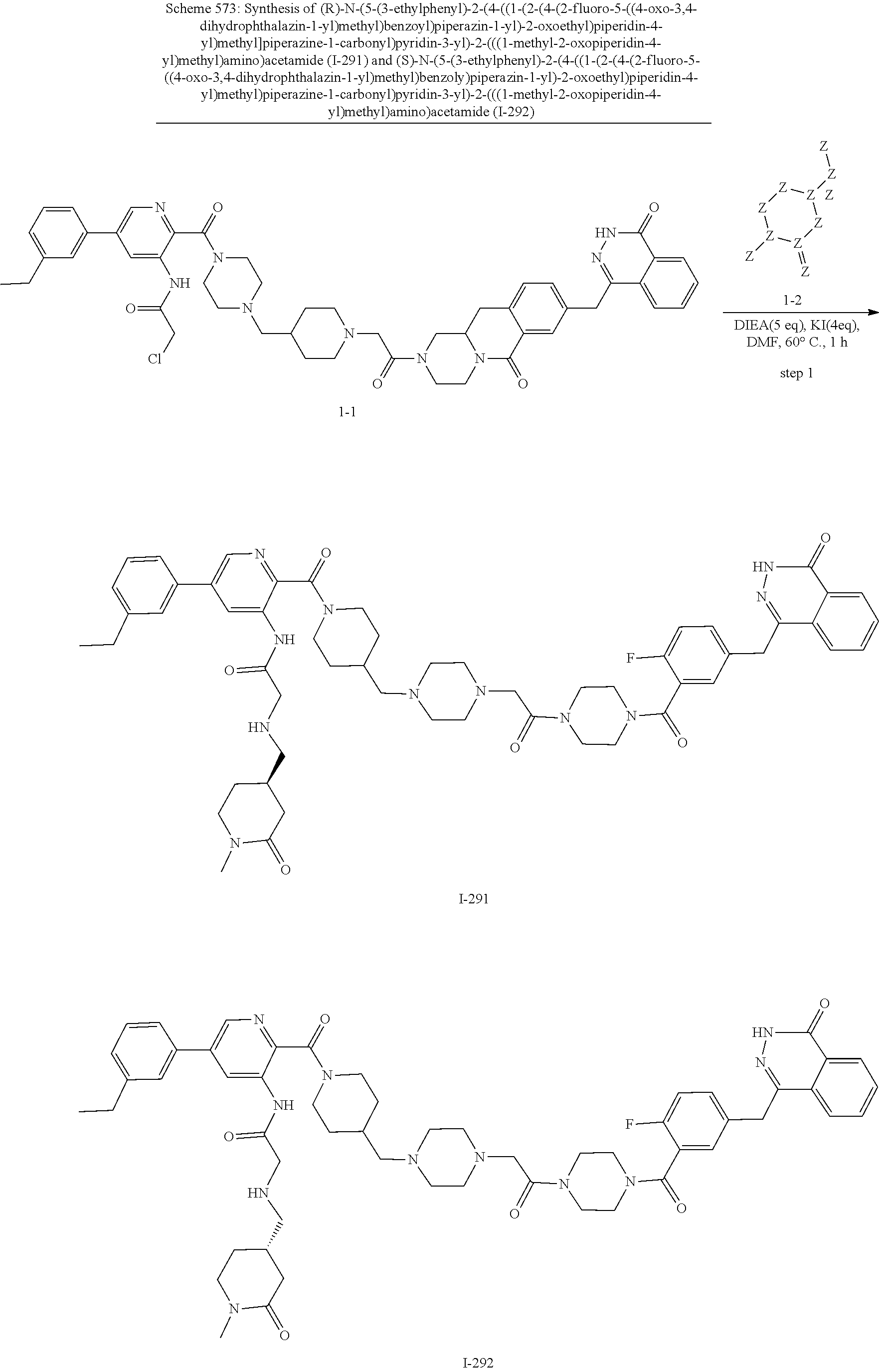

To a solution of Intermediate 1-1 (100 mg, 112.30 μmol, 1 eq) and Intermediate 1-2 (30.10 mg, 168.46 μmol, 1.5 eq, HCl salt) in DMF (1 mL) was added DIEA (72.57 mg, 561.52 μmol, 97.81 μL, 5 eq) and KI (74.57 mg, 449.22 μmol, 4 eq). The mixture was stirred at 60° C. for 1 hr. The reaction mixture was filtered and concentrated under reduced pressure to give a residue. The residue was purified by prep-HPLC (column: Phenomenex Luna C18 150*25 mm*10 um; mobile phase: [water (TFA)-ACN]; gradient: 12%-42% B over 11 min), then concentrated to remove organic solvents and lyophilized to give product, then the product was purified by SFC separation (column: DAICEL CHIRALPAK IK (250 mm*50 mm, 10 um); mobile phase: [Hexane-EtOH (0.1% NH₃·H₂O)]; B %: 30%, isocratic elution mode), then concentrated to remove organic solvents and lyophilized to give Peak 1 and Peak 2. Peak 1 was purified by prep-HPLC (column: Phenomenex Luna C18 150*25 mm*10 um; mobile phase: [water (TFA)-ACN]; gradient: 12% 42% B over 11 min), then concentrated to remove organic solvents and lyophilized to give I-291. Peak 2 was purified by prep-HPLC (column: Phenomenex Luna C18 150*25 mm*10 um; mobile phase: [water (TFA)-ACN]; gradient: 12%-42% B over 11 min), then concentrated to remove organic solvents and lyophilized to give I-292. I-291 (9.96 mg, 9.90 μmol, 8.81% yield, 99% purity) was obtained as a white solid. I-292 (10.02 mg, 10.06 μmol, 8.96% yield, 100% purity) was obtained as a white solid.

I-291 SFC: Rt=2.991 min, ee value=99.58%. 1-292 SFC: Rt=3.679 min, ee value=96.104%.

Rt=0.406 min, [M+H]⁺=996.6 Rt=0.427 min, [M+H]⁺=996.5

Rt=0.433 min, [M+H]⁺=996.5

Rt=0.415 min, [M+H]⁺=996.8 Rt=0.415 min, [M+H]⁺=996.8

¹H NMR (400 MHz, METHANOL-d₄) δ=8.72 (d, J=1.6 Hz, 1H), 8.37 (d, J=7.6 Hz, 1H), 8.20 (s, 1H), 7.97-7.94 (m, 1H), 7.91-7.81 (m, 2H), 7.55-7.48 (m, 3H), 7.48-7.42 (m, 1H), 7.40-7.32 (m, 2H), 7.20-7.15 (m, 1H), 4.94-4.91 (m, 1H), 4.39 (s, 2H), 4.33-4.22 (m, 2H), 4.10 (s, 2H), 3.98-3.87 (m, 2H), 3.85-3.66 (m, 5H), 3.60-3.39 (m, 9H), 3.39-3.33 (m, 3H), 3.25-3.05 (m, 6H), 2.95 (s, 3H), 2.78-2.72 (m, 2H), 2.62-2.57 (m, 1H), 2.41-2.27 (m, 2H), 2.22-2.04 (m, 4H), 1.82-1.61 (m, 3H), 1.31-1.27 (m, 3H)

¹H NMR (400 MHz, METHANOL-d₄) δ=8.72 (d, J=1.6 Hz, 1H), 8.40-8.34 (m, 1H), 8.20 (d, J=1.6 Hz, 1H), 7.99-7.92 (m, 1H), 7.91-7.81 (m, 2H), 7.55-7.48 (m, 3H), 7.48-7.42 (m, 1H), 7.40-7.32 (m, 2H), 7.20-7.15 (m, 1H), 4.83 (d, J=3.2 Hz, 1H), 4.39 (s, 2H), 4.33-4.22 (m, 2H), 4.10 (s, 2H), 3.92 (br s, 2H), 3.85-3.66 (m, 5H), 3.60-3.39 (m, 9H), 3.39-3.33 (m, 3H), 3.23-3.05 (m, 6H), 2.95 (s, 3H), 2.78-2.72 (m, 2H), 2.64-2.56 (m, 1H), 2.41-2.27 (m, 2H), 2.22-2.05 (m, 4H), 1.81-1.61 (m, 3H), 1.31-1.27 (m, 3H)

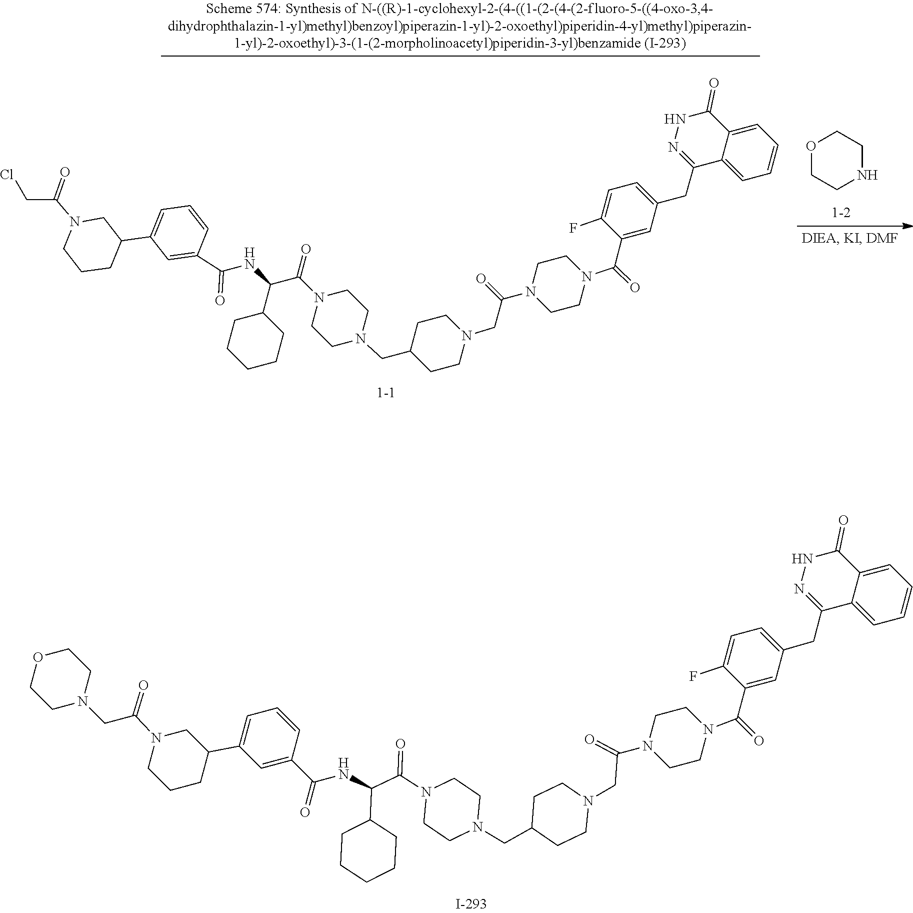

To a solution of Intermediate 1-1 (50 mg, 50.37 μmol, 1 eq) and Intermediate 1-2 (6.58 mg, 75.56 μmol, 6.65 μL, 1.5 eq) in DMF (1 mL) was added KI (33.45 mg, 201.49 μmol, 4 eq) and DIEA (26.04 mg, 201.49 μmol, 35.09 μL, 4 eq). The mixture was stirred at 60° C. for 1 hr. The reaction was filtered and concentrated under reduced pressure to give a residue. The residue was purified by prep-HPLC (column: YMC-Actus Triart C18 150*30 mm*7 um; mobile phase: [water (FA)-ACN]; gradient: 10%-40% B over 10 min). The eluent was concentrated under reduced pressure to remove ACN, then lyophilized to afford I-293 (36 mg, 33.05 μmol, 65.61% yield, 100% purity, FA salt) as a white solid. LCMS (Method E): Rt=0.414 min, [M+H]⁺=1043.6. SFC: Rt=5.111 min, 6.234 min. ¹H NMR (400 MHz, METHANOL-d₄) δ=8.37 (d, J=7.6 Hz, 1H), 8.09 (s, 1H), 7.99-7.75 (m, 4H), 7.72 (d, J=6.4 Hz, 1H), 7.60-7.42 (m, 3H), 7.42-7.31 (m, 1H), 7.19-7.15 (m, 1H), 4.63-4.48 (m, 1H), 4.40 (s, 2H), 4.36-4.04 (m, 4H), 4.04-3.88 (m, 5H), 3.87-3.71 (m, 6H), 3.69-3.46 (m, 6H), 3.40 (br s, 4H), 3.28-3.17 (m, 3H), 3.10-2.86 (m, 2H), 2.84-2.62 (m, 5H), 2.52 (d, J=2.8 Hz, 2H), 2.15-2.01 (m, 4H), 1.90 (d, J=9.6 Hz, 4H), 1.83-1.45 (m, 8H), 1.40-1.04 (m, 6H).

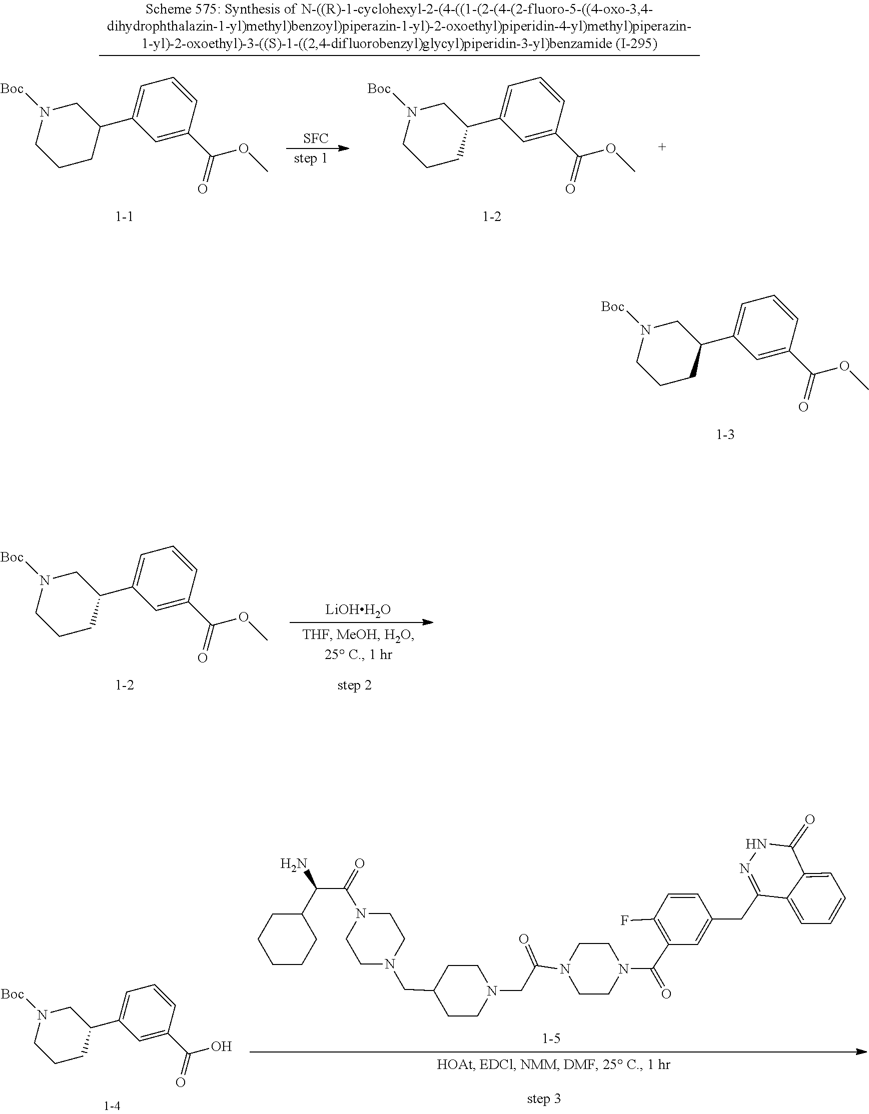

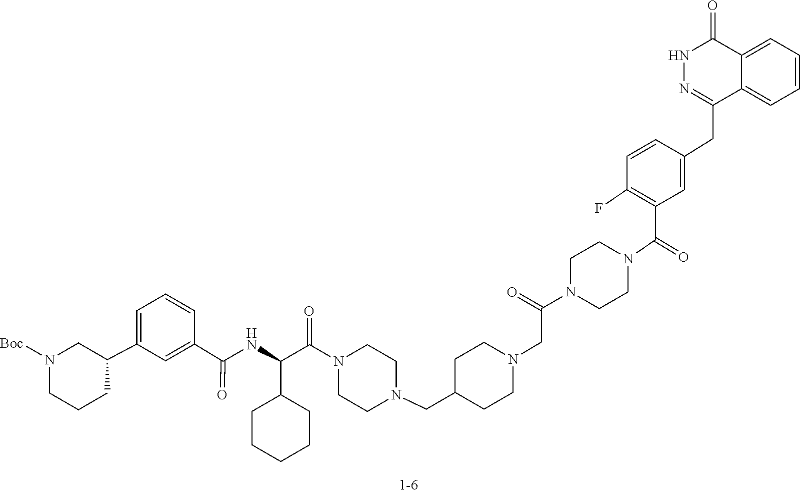 [SMILES: CC(C)(C)OC(=O)N1CCC[C@@H](c2cccc(C(=O)N[C@@H](C(=O)N3CCN(CC4CCN(CC(=O)N5CCN(C(=O)c6cc(Cc7n[nH]c(=O)c8ccccc78)ccc6F)CC5)CC4)CC3)C3CCCCC3)c2)C1]

### Step 1: Synthesis of Intermediate 1-2 and Intermediate 1-3

Intermediate 1-1 was purified by prep-HPLC (column: REGIS(S,S) WHELK-O1 (250 mm*25 mm, 10 um); mobile phase: [CO₂-i-PrOH (0.100NH₃H₂O)]; B %: 20%, isocratic elution mode) and the eluent was concentrated under reduced pressure to give product. Intermediate 1-2 (3 g, 9.39 mmol, 42.86% yield) was obtained as yellow oil. Intermediate 1-3 (3 g, 9.39 mmol, 42.86% yield) was obtained as yellow oil. LCMS (Method D): Rt=0.500 min, [M+Na]⁺=342.2. LCMS (Method D): Rt=0.501 min, [M+Na]⁺=342.2. ¹H NMR (400 MHz, METHANOL-d₄) δ=7.92 (s, 1H), 7.88 (d, J=7.6 Hz, 1H), 7.52 (d, J=7.6 Hz, 1H), 7.47-7.41 (m, 1H), 4.09 (d, J=12.4 Hz, 2H), 2.86 (s, 2H), 2.76-2.70 (m, 1H), 2.01 (d, J=12.0 Hz, 1H), 1.81-1.70 (m, 2H), 1.63-1.56 (m, 1H), 1.47 (s, 9H). ¹H NMR (400 MHz, METHANOL-d4) δ=7.93 (s, 1H), 7.89 (d, J=7.6 Hz, 1H), 7.53 (d, J=7.6 Hz, 1H), 7.47-7.41 (m, 1H), 4.10 (d, J=12.0 Hz, 2H), 2.85 (d, J=9.2 Hz, 2H), 2.77-2.70 (m, 1H), 2.01 (d, J=12.0 Hz, 1H), 1.81-1.71 (m, 2H), 1.64-1.56 (m, 1H), 1.47 (s, 9H).

SFC: Rt=1.272 min

SFC: Rt=1.381 min

### Step 2: Synthesis of Intermediate 1-4

To a solution of Intermediate 1-2 (1.2 g, 3.76 mmol, 1 eq) in THF (4 mL), MeOH (4 mL) and H₂O (4 mL) was added LiOH·H₂O (472.99 mg, 11.27 mmol, 3 eq). The mixture was stirred at 25° C. for 1 hr. The pH was acidified to 5-6 with 1N HCl, and the mixture was washed with water (5 mL) and extract with EA (8 mL*3). The combined organic layers were dried over sodium sulfate, filtered and concentrated under reduced pressure to give a residue. Intermediate 1-4 (1.1 g, 3.57 mmol, 94.92% yield, 99% purity) was obtained as yellow oil. LCMS (Method D): Rt=0.447 min, [M+Na]⁺=328.1. ¹H NMR (400 MHz, METHANOL-d₄) δ=7.93 (s, 1H), 7.89 (d, J=7.6 Hz, 1H), 7.50 (s, 1H), 7.45-7.40 (m, 1H), 4.20-4.02 (m, 2H), 2.87 (d, J=11.6 Hz, 2H), 2.77-2.70 (m, 1H), 2.05-1.99 (m, 1H), 1.82-1.73 (m, 2H), 1.63-1.56 (m, 1H), 1.47 (s, 9H).

### Step 3: Synthesis of Intermediate 1-6

To a solution of Intermediate 1-4 (1.1 g, 3.60 mmol, 1 eq) in DMF (10 mL) was added EDCI (2.07 g, 10.81 mmol, 3 eq) and HOAt (490.30 mg, 3.60 mmol, 503.91 μL, 1 eq) for 10 min. Then Intermediate 1-5 (2.76 g, 3.60 mmol, 1 eq, HCl salt) and NMM (1.82 g, 18.01 mmol, 1.98 mL, 5 eq) were added. The mixture was stirred at 25° C. for 50 min. The mixture was washed with water (15 mL) and extracted with EA (20 mL*3). The combined organic layers were dried over sodium sulfate, filtered and concentrated under reduced pressure to give a residue. The residue was purified by flash silica gel chromatography (ISCO; 80 g SepaFlash Silica Flash Column, Eluent of 0˜100% MeOH/EA @100 mL/min) and then the eluent was concentrated in vacuum to give product. Intermediate 1-6 (2.4 g, 2.20 mmol, 60.97% yield, 93% purity) was obtained as a yellow solid. LCMS (Method D): Rt=0.389 min, [(M-Boc)/2+H]⁺=458.9. ¹H NMR (400 MHz, METHANOL-d₄) δ=8.39-8.33 (m, 1H), 7.97-7.92 (m, 1H), 7.90-7.85 (m, 1H), 7.85-7.81 (m, 1H), 7.75 (s, 1H), 7.70 (d, J=7.6 Hz, 1H), 7.44-7.41 (m, 3H), 7.39 (d, J=8.0 Hz, 1H), 7.16-7.13 (m, 1H), 4.90 (d, J=8.8 Hz, 1H), 4.38 (s, 2H), 4.15-4.09 (m, 3H), 3.85-3.76 (m, 2H), 3.76-3.65 (m, 5H), 3.57-3.49 (m, 3H), 3.34 (s, 2H), 3.27 (s, 1H), 3.00-2.92 (m, 2H), 2.85 (d, J=12.0 Hz, 2H), 2.72-2.69 (m, 1H), 2.52-2.42 (m, 3H), 2.38 (s, 1H), 2.27-2.13 (m, 4H), 2.01-1.95 (m, 1H), 1.93-1.86 (m, 2H), 1.81-1.73 (m, 6H), 1.69 (d, J=12.0 Hz, 2H), 1.63-1.55 (m, 2H), 1.46 (s, 9H), 1.35-1.25 (m, 4H), 1.22-1.16 (m, 1H), 1.15-1.05 (m, 2H). ¹⁹F NMR (376 MHz, METHANOL-d₄) δ=−120.747. SFC: Rt=3.342 min, 3.721 min.

### Step 4: Synthesis of Intermediate 1-7

To a solution of Intermediate 1-6 (2.4 g, 2.36 mmol, 1 eq) in DCM (24 mL) was added HCl/dioxane (2 M, 8 mL, 6.77 eq). The mixture was stirred at 25° C. for 0.5 hr. The reaction mixture was concentrated in vacuum. The crude product was used in the next step without further purification. Intermediate 1-7 (2.4 g, crude, HCl salt) was obtained as a yellow solid. LCMS (Method D): Rt=0.288 min, [M+H]⁺=916.5.

### Step 5: Synthesis of Intermediate 1-9

To a solution of Intermediate 1-7 (2.2 g, 2.40 mmol, 1 eq) in DCM (22 mL) was added TEA (728.98 mg, 7.20 mmol, 1.00 mL, 3 eq) and Intermediate 1-8 (325.46 mg, 2.88 mmol, 229.52 μL, 1.2 eq) at 0° C. The mixture was stirred at 0° C. for 15 min. The mixture was washed with water (20 mL) and extracted with DCM (30 mL*3). The combined organic layers were dried over sodium sulfate, filtered and concentrated under reduced pressure to give a residue. The residue was purified by flash silica gel chromatography (ISCO; 80 g SepaFlash Silica Flash Column, Eluent of 0˜100% MeOH/EA @100 mL/min) and then the eluent was concentrated in vacuum to give Intermediate 1-9 (1.5 g, 1.28 mmol, 53.49% yield, 85% purity) as a yellow solid. LCMS (Method D): Rt=0.343 min, [M+H]⁺=992.4.

### Step 6: Synthesis of I-295

To a solution of Intermediate 1-9 (600 mg, 604.46 μmol, 1 eq) and Intermediate 1-10 (86.52 mg, 604.46 μmol, 1 eq) in DMF (6 mL) was added DIEA (234.36 mg, 1.81 mmol, 315.85 μL, 3 eq). The mixture was stirred at 40° C. for 12 hr. The reaction mixture was concentrated in vacuum. The residue was purified by prep-HPLC (0.1% NH₃·H₂O condition), the eluent was concentrated under reduced pressure to remove ACN and lyophilized to give product. I-295 (198.38 mg, 172.74 μmol, 28.58% yield, 95.722% purity) was obtained as a white solid. LCMS (Method D): Rt=0.317 min, [M+H]⁺=1099.2. SFC: Rt=4.102 min. ¹H NMR (400 MHz, METHANOL-d₄) δ=8.36 (d, J=7.6 Hz, 1H), 7.97-7.92 (m, 1H), 7.84-7.82 (m, 2H), 7.76 (s, 1H), 7.73-7.69 (m, 1H), 7.51-7.41 (m, 4H), 7.38 (s, 1H), 7.16-7.15 (m, 1H), 6.99-6.87 (m, 2H), 4.91-4.89 (m, 1H), 4.55 (d, J=10.8 Hz, 1H), 4.38 (s, 2H), 3.87 (s, 1H), 3.81 (d, J=11.2 Hz, 4H), 3.76-3.69 (m, 4H), 3.66 (d, J=1.6 Hz, 1H), 3.58-3.51 (m, 4H), 3.49-3.39 (m, 1H), 3.36-3.32 (m, 1H), 3.30-3.26 (m, 1H), 3.24 (s, 1H), 3.18 (s, 1H), 3.16-3.05 (m, 1H), 2.90-2.86 (m, 2H), 2.80-2.66 (m, 2H), 2.52-2.35 (m, 4H), 2.20-2.18 (m, 2H), 2.09-2.01 (m, 3H), 1.93-1.81 (m, 4H), 1.77 (d, J=9.6 Hz, 4H), 1.69 (d, J=11.6 Hz, 2H), 1.62-1.50 (m, 2H), 1.38-1.20 (m, 5H), 1.19-1.04 (m, 3H). ¹⁹F NMR (376 MHz, METHANOL-d₄) δ=−113.360, −116.230, −120.696.

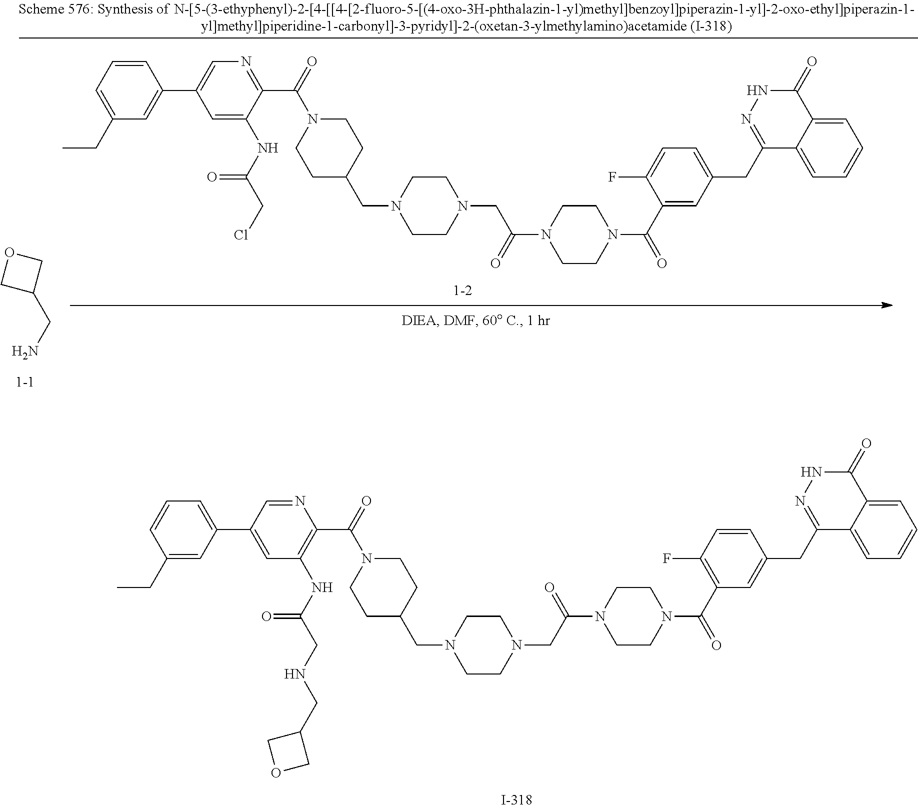 [SMILES: NCC1COC1 ; CCc1cccc(-c2cnc(C(=O)N3CCC(CN4CCN(CC(=O)N5CCN(C(=O)c6cc(Cc7n[nH]c(=O)c8ccccc78)ccc6F)CC5)CC4)CC3)c(NC(=O)CCl)c2)c1 ; CCc1cccc(-c2cnc(C(=O)N3CCC(CN4CCN(CC(=O)N5CCN(C(=O)c6cc(Cc7n[nH]c(=O)c8ccccc78)ccc6F)CC5)CC4)CC3)c(NC(=O)CNCC3COC3)c2)c1]

To a mixture of Intermediate 1-2 (100 mg, 112.30 μmol, 1 eq) and Intermediate 1-1 (11.74 mg, 134.76 μmol, 1.2 eq) in DMF (1 mL) was added DIEA (29.03 mg, 224.61 μmol, 39.12 μL, 2 eq). The mixture was stirred at 60° C. for 1 hr. The reaction was poured into H₂O (2 mL) and extracted with EA (3 mL*3). The combined organic layers were dried over Na₂SO₄, filtered and concentrated under reduced pressure to give the crude product. The crude product was purified by reversed-phase HPLC (0.1% FA condition) followed by lyophilization to give I-318 (28 mg, 26.59 μmol, 23.67% yield, 93.733% purity, FA salt) as a white solid. LCMS (Method D): Rt=0.354 min, [M+H]⁺=941.5. ¹H NMR (400 MHz, METHANOL-d₄) δ=8.76 (s, 1H), 8.60 (s, 1H), 8.40 (s, 1H), 8.37 (d, J=7.6 Hz, 1H), 8.00-7.92 (m, 1H), 7.91-7.81 (m, 2H), 7.56-7.48 (m, 3H), 7.43 (t, J=7.6 Hz, 1H), 7.40-7.29 (m, 2H), 7.17 (t, J=8.4 Hz, 1H), 4.73-4.66 (m, 2H), 4.51 (t, J=6.0 Hz, 2H), 4.39 (s, 2H), 3.88-3.64 (m, 6H), 3.54-3.43 (m, 5H), 3.36 (s, 1H), 3.27-3.12 (m, 3H), 3.01 (d, J=7.2 Hz, 2H), 2.97-2.89 (m, 1H), 2.79-2.70 (m, 7H), 2.56-2.43 (m, 2H), 2.06-1.91 (m, 2H), 1.82-1.72 (m, 1H), 1.40-1.33 (m, 5H), 1.29 (t, J=7.2 Hz, 4H). ¹⁹F NMR (377 MHz, METHANOL-d₄) δ=−120.794.

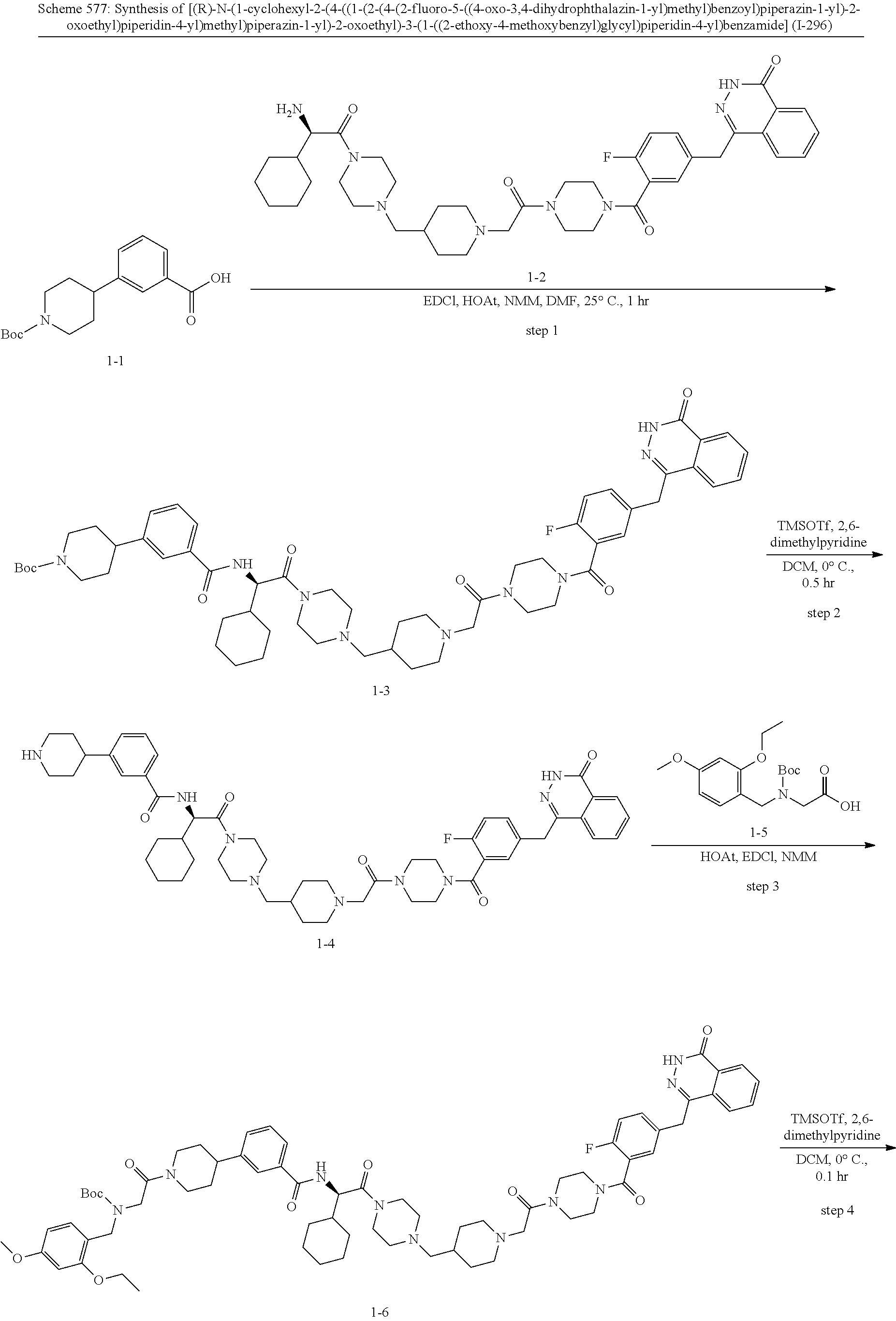

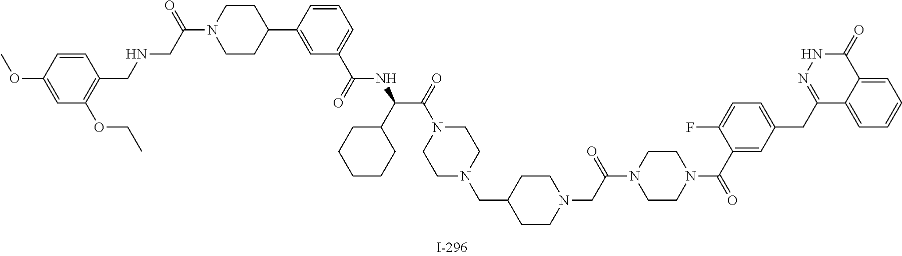 [SMILES: CCOc1cc(OC)ccc1CNCC(=O)N1CCC(c2cccc(C(=O)N[C@@H](C(=O)N3CCN(CC4CCN(CC(=O)N5CCN(C(=O)c6cc(Cc7n[nH]c(=O)c8ccccc78)ccc6F)CC5)CC4)CC3)C3CCCCC3)c2)CC1]

### Step 1: Synthesis of Intermediate 1-3.

To a solution of Intermediate 1-1 (300 mg, 982.42 μmol, 1 eq) in DMF (4 mL) was added HOAt (133.72 mg, 982.42 μmol, 137.43 μL, 1 eq) and EDCI (564.99 mg, 2.95 mmol, 3 eq), then Intermediate 1-2 (751.90 mg, 982.42 μmol, 1 eq, HCl salt) and NMM (496.84 mg, 4.91 mmol, 540.05 μL, 5 eq) were added. The mixture was stirred at 25° C. for 1 hr. The reaction mixture was diluted with H₂O (5 mL) and extracted with EA (5 mL*3). The combined organic layers were washed with brine (5 mL*3), dried over Na₂SO₄, filtered and concentrated under reduced pressure to give a residue. The residue was purified by flash silica gel chromatography (ISCO; 20 g SepaFlash Silica Flash Column, Eluent of 0˜15% Methanol/Ethyl acetate gradient @60 mL/min), the eluent was concentrated under reduced pressure to give a residue. Intermediate 1-3 (470 mg, 368.84 μmol, 37.54% yield, 79.752% purity) was obtained as a yellow solid. LCMS (Method D): Retention time: 0.431 min, [M+H]⁺=1016.6. ¹H NMR (400 MHz, METHANOL-d₄) δ=8.37 (d, J=7.6 Hz, 1H), 7.98-7.92 (m, 1H), 7.91-7.79 (m, 2H), 7.74-7.62 (m, 2H), 7.53-7.34 (m, 4H), 7.21-7.09 (m, 1H), 4.90 (d, J=8.8 Hz, 1H), 4.38 (s, 2H), 4.22 (d, J=12.8 Hz, 2H), 3.84-3.65 (m, 7H), 3.58-3.49 (m, 3H), 3.06-2.68 (m, 6H), 2.55-2.34 (m, 4H), 2.25-2.12 (m, 4H), 1.92-1.74 (m, 9H), 1.71-1.57 (m, 6H), 1.48 (s, 9H), 1.35-1.18 (m, 6H), 1.15-1.05 (m, 2H). ¹⁹F NMR (376 MHz, METHANOL-d₄) δ=−120.76.

### Step 2: Synthesis of Intermediate 1-4.

To a solution of Intermediate 1-3 (470 mg, 462.49 μmol, 1 eq) in DCM (5 mL) was added 2,6-dimethylpyridine (14.87 mg, 138.75 μmol, 16.16 μL, 0.3 eq) and trimethylsilyl trifluoromethanesulfonate (154.19 mg, 693.73 μmol, 125.35 μL, 1.5 eq). The mixture was stirred at 0° C. for 0.5 hr. The reaction mixture was concentrated to give a residue. The crude product was used in the next step without further purification. Intermediate 1-4 (450 mg, crude) was obtained as a white solid. LCMS (Method D): Retention time: 0.288 min, [M+H]⁺=916.5.

### Step 3: Synthesis of Intermediate 1-6.

To a solution of Intermediate 1-5 (92.61 mg, 272.89 μmol, 1 eq) in DMF (2 mL) was added HOAt (37.14 mg, 272.89 μmol, 38.17 μL, 1 eq) and EDCI (156.94 mg, 818.66 μmol, 3 eq). Then Intermediate 1-4 (250 mg, 272.89 μmol, 1 eq) and NMM (138.01 mg, 1.36 mmol, 150.01 μL, 5 eq) was added. The mixture was stirred at 25° C. for 1 hr. The reaction mixture diluted with H₂O (20 mL) and filtered to give a residue. The residue was purified by prep-HPLC (FA condition), the eluent was concentrated and lyophilized to give the desired product. Intermediate 1-6 (180 mg, 140.24 μmol, 51.39% yield, 100% purity, FA salt) was obtained as a white solid. LCMS (Method D): Retention time: 0.418 min, [M+H]⁺=1237.7. SFC: Retention time: 1.060 min. ¹H NMR (400 MHz, METHANOL-d₄) δ=8.37 (d, J=7.2 Hz, 1H), 7.94 (d, J=6.4 Hz, 1H), 7.89-7.79 (m, 2H), 7.76-7.62 (m, 2H), 7.51 (s, 1H), 7.46-7.32 (m, 3H), 7.21-7.10 (m, 2H), 6.54-6.45 (m, 2H), 4.44-4.35 (m, 4H), 4.19-4.09 (m, 3H), 4.07-4.00 (m, 3H), 3.94-3.81 (m, 4H), 3.78-3.69 (m, 7H), 3.59-3.48 (m, 6H), 3.18-3.10 (m, 2H), 3.04-2.97 (m, 2H), 2.92-2.85 (m, 1H), 2.81-2.76 (m, 1H), 2.60-2.48 (m, 3H), 2.45-2.38 (m, 1H), 2.31 (d, J=6.4 Hz, 2H), 2.07-1.99 (m, 2H), 1.90 (s, 3H), 1.87 (s, 2H), 1.81-1.74 (m, 2H), 1.72-1.65 (m, 3H), 1.48 (s, 9H), 1.43-1.35 (m, 6H), 1.33-1.19 (m, 5H), 1.15-1.06 (m, 2H).

### Step 4: Synthesis of I-296

To a solution of Intermediate 1-6 (60 mg, 46.75 μmol, 1 eq, FA salt) in DCM (0.6 mL) was added 2,6-dimethylpyridine (1.50 mg, 14.02 μmol, 1.63 μL, 0.3 eq) and trimethylsilyl trifluoromethanesulfonate (15.58 mg, 70.12 μmol, 12.67 μL, 1.5 eq). The mixture was stirred at 0° C. for 0.1 hr. The reaction mixture was concentrated under reduced pressure to give a residue. The residue was purified by prep-HPLC (basic condition), the eluent was concentrated and lyophilized to give the desired product. I-296 (22.43 mg, 19.64 μmol, 42.01% yield, 99.591% purity) was obtained as a white solid. LCMS (Method D): Retention time: 0.373 min, [M+H]⁺=1137.6. SFC: Retention time: 0.976 min, ¹H NMR (400 MHz, METHANOL-d₄) δ=8.40-8.35 (m, 1H), 7.99-7.93 (m, 1H), 7.90-7.81 (m, 2H), 7.72-7.64 (m, 2H), 7.50-7.35 (m, 4H), 7.20-7.11 (m, 2H), 6.56-6.44 (m, 2H), 4.91-4.89 (m, 1H), 4.66-4.56 (m, 2H), 4.39 (s, 2H), 4.13-4.03 (m, 2H), 3.94-3.78 (m, 5H), 3.76-3.65 (m, 8H), 3.59-3.46 (m, 5H), 3.27-3.13 (m, 3H), 2.95-2.84 (m, 3H), 2.79-2.68 (m, 1H), 2.54-2.32 (m, 4H), 2.25-2.16 (m, 2H), 2.12-2.00 (m, 2H), 1.94-1.83 (m, 4H), 1.81-1.73 (m, 4H), 1.71-1.52 (m, 5H), 1.48-1.41 (m, 3H), 1.35-1.18 (m, 5H), 1.15-1.03 (m, 2H). ¹⁹F NMR (376 MHz, METHANOL-d₄) δ=−120.792.

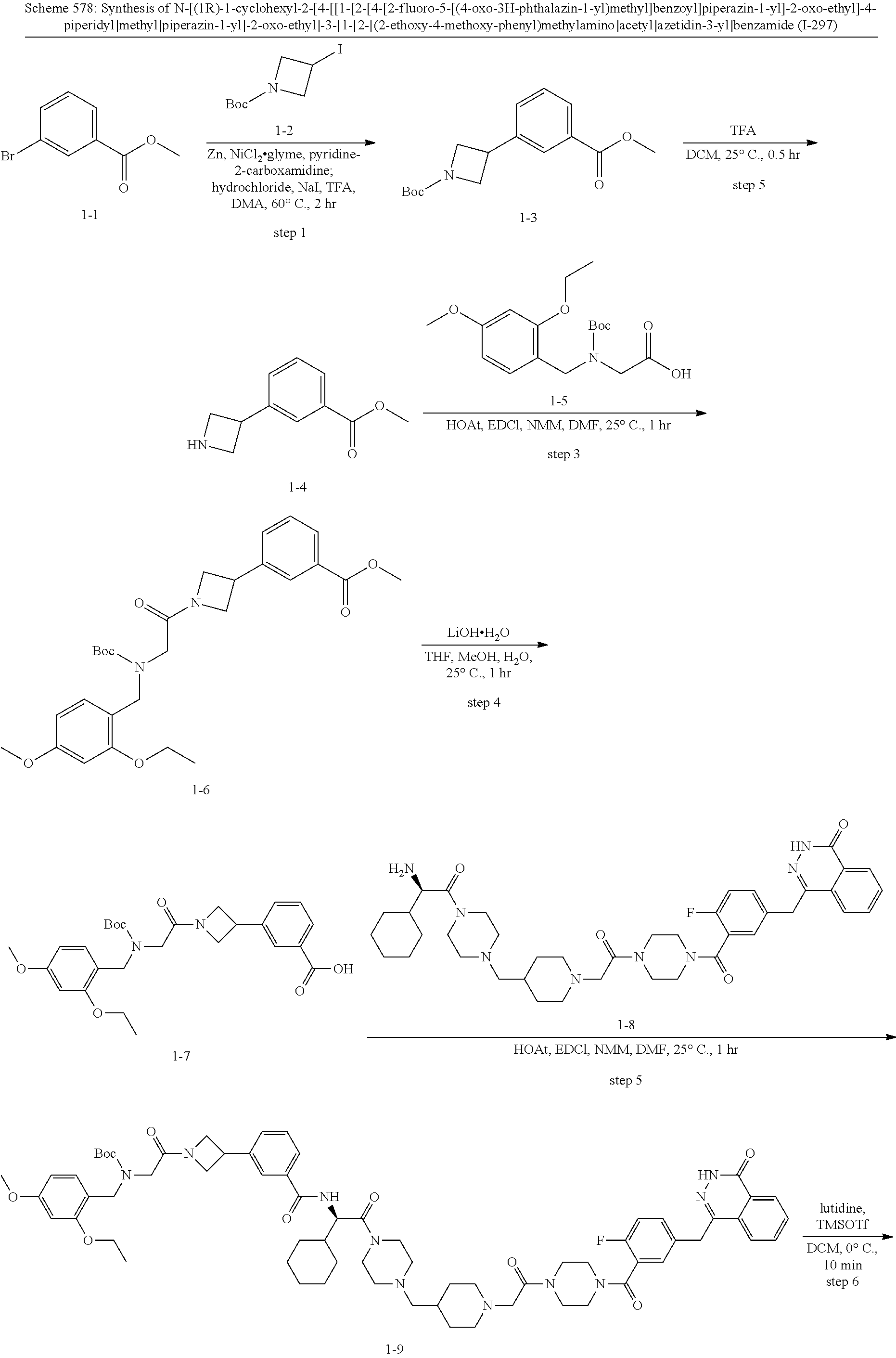

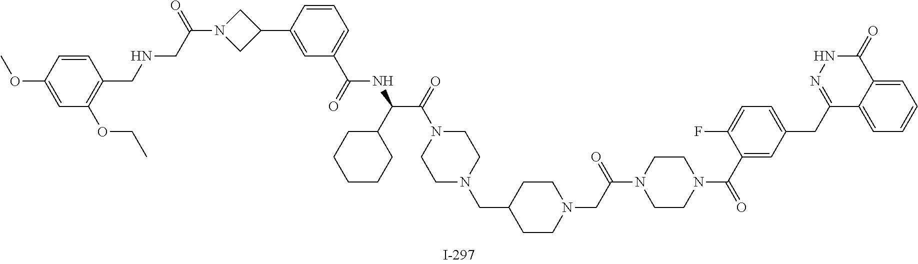 [SMILES: CCOc1cc(OC)ccc1CNCC(=O)N1CC(c2cccc(C(=O)N[C@@H](C(=O)N3CCN(CC4CCN(CC(=O)N5CCN(C(=O)c6cc(Cc7n[nH]c(=O)c8ccccc78)ccc6F)CC5)CC4)CC3)C3CCCCC3)c2)C1]

### Step 1: Synthesis of Intermediate 1-3

A mixture of Intermediate 1-1 (1.14 g, 5.30 mmol, 1.5 eq) and Intermediate 1-2 (1 g, 3.53 mmol, 1 eq) in DMA (10 mL) was added NiCl₂. glyme (77.61 mg, 353.22 μmol, 0.1 eq), pyridine-2-carboxamidine;hydrochloride (55.67 mg, 353.22 μmol, 0.1 eq), NaI (264.72 mg, 1.77 mmol, 0.5 eq), TFA (40.27 mg, 353.22 μmol, 26.24 μL, 0.1 eq) and Zn (890 mg, 13.61 mmol, 3.85 eq) and then the mixture was stirred at 60° C. for 2 hr under N₂ atmosphere. The reaction mixture was filtered and diluted with H₂O (20 mL) and then extracted with EA (25 mL*3). The combined organic layers were dried over sodium sulfate, filtered and concentrated under reduced pressure to give a residue. The solid was treated with 20 mL of 1M HCl. The residue was purified by column chromatography (SiO₂, PE/EA=1/0 to 0/1, Rf=0.7, PE:EA=3:1) to give product and the eluent was concentrated in vacuum. Intermediate 1-3 (840 mg, 2.80 mmol, 79.18% yield, 97% purity) was obtained as colorless oil. LCMS (Method D): Rt=0.515 min, [M+Na]⁺=314.1. ¹H NMR (400 MHz, METHANOL-d₄) δ=7.99 (s, 1H), 7.91 (d, J=7.6 Hz, 1H), 7.59 (d, J=7.6 Hz, 1H), 7.51-7.44 (m, 1H), 4.37-4.35 (m, 2H), 3.97-3.92 (m, 2H), 3.91 (s, 3H), 3.89-3.83 (m, 1H), 1.47 (s, 9H).

### Step 2: Synthesis of Intermediate 1-4

To a solution of Intermediate 1-3 (800 mg, 2.75 mmol, 1 eq) in DCM (8 mL) was added TFA (2.46 g, 21.54 mmol, 1.6 mL). The mixture was stirred at 25° C. for 0.5 hr. The reaction mixture was concentrated in vacuum. The crude product was used in the next step without further purification. Intermediate 1-4 (900 mg, crude, TFA salt) was obtained as a yellow oil. LCMS (Method D): Rt=0.225 min, [M+H]⁺=192.1. ¹H NMR (400 MHz, METHANOL-d4) δ=8.06 (s, 1H), 7.99 (d, J=7.6 Hz, 1H), 7.67-7.61 (m, 1H), 7.57-7.52 (m, 1H), 4.44-4.38 (m, 2H), 4.34-4.31 (m, 1H), 4.30-4.25 (m, 2H), 3.93 (s, 3H).

### Step 3: Synthesis of Intermediate 1-6

To a solution of Intermediate 1-5 (333.55 mg, 982.80 μmol, 1 eq) in DMF (3 mL) was added EDCI (565.21 mg, 2.95 mmol, 3 eq) and HOAt (133.77 mg, 982.80 μmol, 137.48 μL, 1 eq) and the mixture was stirred for 10 min. Then Intermediate 1-4 (300 mg, 982.80 μmol, 1 eq, TFA salt) and NMM (497.04 mg, 4.91 mmol, 540.26 μL, 5 eq) were added. The mixture was stirred at 25° C. for 50 min. The mixture was diluted with EA, washed with water (5 mL) and extracted with EA (8 mL*3). The combined organic layers were dried over sodium sulfate, filtered and concentrated under reduced pressure to give a residue. The residue was purified by column chromatography (SiO₂, PE/EA=1/0 to 0/1, Rf=0.8, PE:EA=0:1) to give product and the eluent was concentrated in vacuum. Intermediate 1-6 (500 mg, 955.92 μmol, 97.26% yield, 98% purity) was obtained as yellow. LCMS (Method D): Rt=0.499 min, [M+H]⁺=513.3. ¹H NMR (400 MHz, METHANOL-d₄) δ=7.99 (s, 1H), 7.93 (d, J=7.6 Hz, 1H), 7.59-7.49 (m, 1H), 7.52-7.46 (m, 1H), 7.12 (d, J=8.0 Hz, 1H), 6.51-6.45 (m, 2H), 4.60-4.50 (m, 2H), 4.47-4.41 (m, 3H), 4.18-4.11 (m, 1H), 4.07-3.94 (m, 5H), 3.91 (s, 3H), 3.76 (s, 3H), 1.48 (s, 9H), 1.40-1.37 (m, 3H).

### Step 4: Synthesis of Intermediate 1-7

To a solution of Intermediate 1-6 (480 mg, 936.42 μmol, 1 eq) in THF (1.5 mL), MeOH (1.5 mL) and H₂O (1.5 mL) was added LiOH·H₂O (117.89 mg, 2.81 mmol, 3 eq). The mixture was stirred at 25° C. for 1 hr. The pH was acidified to 5-6 with 1N HCl, and the mixture was washed with water (5 mL) and extracted with EA (8 mL*3). The combined organic layers were dried over sodium sulfate, filtered and concentrated under reduced pressure to give a residue. The crude product was used in the next step without further purification. Intermediate 1-7 (470 mg, 923.85 μmol, 98.66% yield, 98% purity) was obtained as a colorless oil. LCMS (Method D): Rt=0.456 min, [M+H]⁺=499.2. ¹H NMR (400 MHz, METHANOL-d₄) δ=8.01 (s, 1H), 7.94 (d, J=7.6 Hz, 1H), 7.57 (d, J=20.0 Hz, 1H), 7.48-7.46 (m, 1H), 7.12 (d, J=8.0 Hz, 1H), 6.52-6.44 (m, 2H), 4.60-4.37 (m, 5H), 4.18-4.13 (m, 1H), 4.07-3.95 (m, 5H), 3.75 (s, 3H), 1.48 (s, 9H), 1.40 (d, J=6.8 Hz, 3H).

### Step 5: Synthesis of Intermediate 1-9

To a solution of Intermediate 1-7 (100 mg, 200.57 μmol, 1 eq) in DMF (1 mL) was added EDCI (115.35 mg, 601.72 μmol, 3 eq) and HOAt (27.30 mg, 200.57 μmol, 28.06 μL, 1 eq). Then Intermediate 1-8 (153.51 mg, 200.57 μmol, 1 eq, HCl salt) and NMM (101.44 mg, 1.00 mmol, 110.26 μL, 5 eq) were added. The mixture was stirred at 25° C. for 1 hr. The mixture was diluted with EA, washed with water (3 mL) and extracted with EA (5 mL*3). The combined organic layers were dried over sodium sulfate, filtered and concentrated under reduced pressure to give a residue. The residue was purified by flash silica gel chromatography (ISCO; 12 g SepaFlash Silica Flash Column, Eluent of 0˜100% MeOH/EA @40 mL/min) and then the eluent was concentrated in vacuum to give product. Intermediate 1-9 (110 mg, 76.40 μmol, 38.09% yield, 84% purity) was obtained as a yellow gum. LCMS (Method D): Rt=0.401 min, [M+H]⁺=1209.2.

### Step 6: Synthesis of I-297

To a solution of Intermediate 1-9 (40 mg, 33.07 μmol, 1 eq) in DCM (0.8 mL) was added lutidine (1.06 mg, 9.92 μmol, 1.16 μL, 0.3 eq) and TMSOTf (11.03 mg, 49.61 μmol, 8.96 μL, 1.5 eq). The mixture was stirred at 0° C. for 10 min. The reaction mixture was concentrated in vacuum. The residue was purified by prep-HPLC (basic condition), the eluent was concentrated under reduced pressure to remove ACN and lyophilized to give product. I-297 (14.87 mg, 12.71 μmol, 38.42% yield, 94.798% purity) was obtained as a white solid. LCMS (Method D): Rt=0.316 min, [M+H]⁺=1109.5. SFC: Rt=1.167 min. ¹H NMR (400 MHz, METHANOL-d₄) δ=8.37 (d, J=7.2 Hz, 1H), 7.98-7.92 (m, 1H), 7.90-7.81 (m, 3H), 7.75 (d, J=7.6 Hz, 1H), 7.56-7.45 (m, 3H), 7.38 (d, J=4.4 Hz, 1H), 7.20-7.12 (m, 2H), 6.55-6.45 (m, 2H), 4.92-4.88 (m, 4H), 4.58 (s, 2H), 4.39 (s, 3H), 4.25-4.19 (m, 1H), 4.10-3.98 (m, 4H), 3.84-3.78 (m, 3H), 3.76 (s, 3H), 3.75-3.67 (m, 5H), 3.59-3.45 (m, 4H), 3.23 (s, 2H), 2.97-2.89 (m, 2H), 2.51-2.34 (m, 4H), 2.24-2.18 (m, 2H), 2.16-2.07 (m, 2H), 1.91-1.85 (m, 2H), 1.77 (s, 4H), 1.71-1.66 (m, 2H), 1.44-1.42 (m, 3H), 1.32-1.22 (m, 5H), 1.17-1.02 (m, 3H). ¹⁹F NMR (376 MHz, ETHANOL-d₄) δ=−80.138, −120.808.

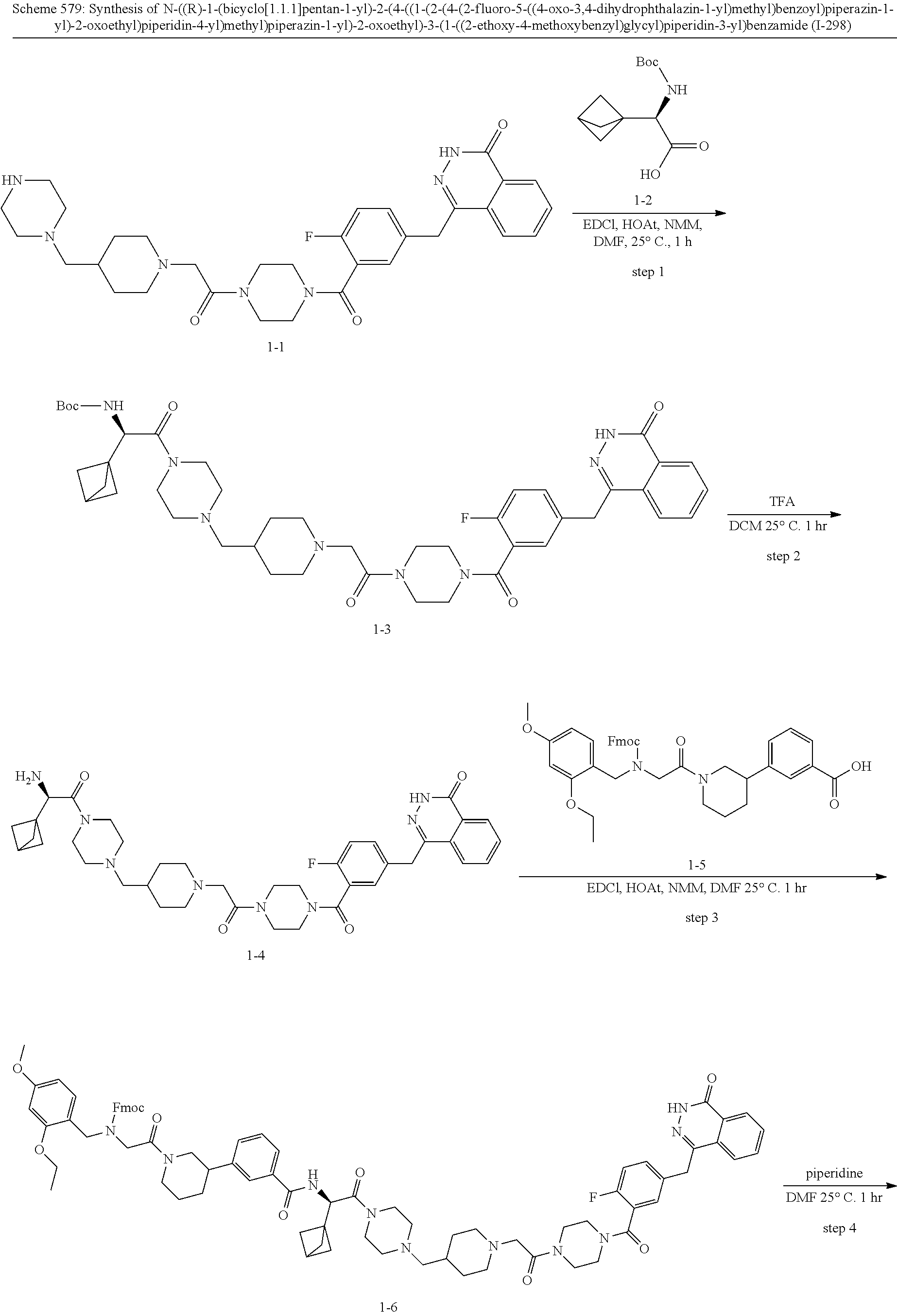

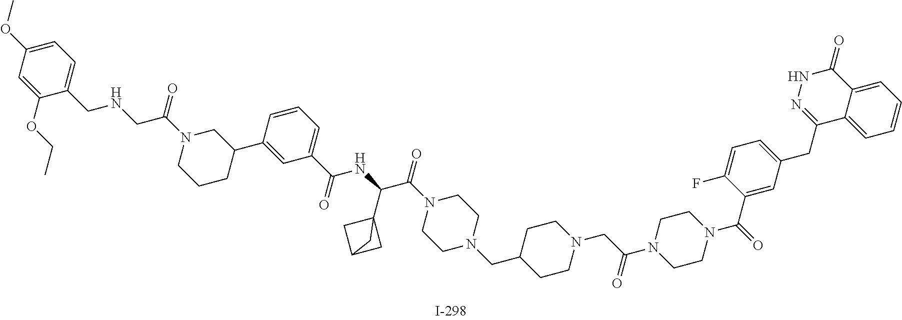 [SMILES: CCOc1cc(OC)ccc1CNCC(=O)N1CCCC(c2cccc(C(=O)N[C@@H](C(=O)N3CCN(CC4CCN(CC(=O)N5CCN(C(=O)c6cc(Cc7n[nH]c(=O)c8ccccc78)ccc6F)CC5)CC4)CC3)C34CC(C3)C4)c2)C1]

### Step 1: Synthesis of Intermediate 1-3

To a solution of Intermediate 1-1 (219.96 mg, 373.01 μmol, 1 eq) and Intermediate 1-2 (90 mg, 373.01 μmol, 1 eq) in DMF (2 mL) was added EDCI (143.01 mg, 746.01 μmol, 2 eq) and HOAt (50.77 mg, 373.01 μmol, 52.18 μL, 1 eq) and NMM (188.64 mg, 1.87 mmol, 205.05 μL, 5 eq). The mixture was stirred at 25° C. for 1 hr. The reaction mixture was filtered and the filtrate was purified by flash silica gel chromatography. The residue was purified by flash silica gel chromatography (ISCO; 20 g SepaFlash Silica Flash Column, Eluent of 0-100% Ethyl acetate/Petroleum ether gradient @30 mL/min) and the eluent was concentrated to give product. Intermediate 1-3 (300 mg, crude) was obtained as a white solid. LCMS (Method D): Retention time: 0.302 min, [M+H]+=813.3. SFC: Retention time: 1.526 min.

### Step 2: Synthesis of Intermediate 1-4

To a solution of Intermediate 1-3 (200 mg, 246.01 μmol, 1 eq) in DCM (2 mL) was added TFA (614.00 mg, 5.38 mmol, 0.4 mL, 21.89 eq). The mixture was stirred at 25° C. for 1 hr. The reaction mixture was concentrated under reduced pressure to give a residue. The crude was used in the next step. Intermediate 1-4 (200 mg, 241.87 μmol, 98.32% yield, TFA salt) was obtained as a white solid. LCMS (Method D): Retention time: 0.253 min, [M+H]+=713.4.

### Step 3: Synthesis of Intermediate 1-6

To a solution of Intermediate 1-4 (200 mg, 241.87 μmol, 1 eq, TFA salt) and Intermediate 1-5 (156.91 mg, 241.87 μmol, 1 eq) in DMF (0.2 mL) was added EDCI (92.73 mg, 483.75 μmol, 2 eq) and HOAt (32.92 mg, 241.87 μmol, 33.83 μL, 1 eq) and NMM (122.33 mg, 1.21 mmol, 132.96 μL, 5 eq). The mixture was stirred at 25° C. for 1 hr. The reaction mixture was diluted with MeOH. The crude product was purified by reversed-phase HPLC (0.1% FA condition), the eluent was concentrated to remove ACN and lyophilized to give product. Intermediate 1-6 (40 mg, 29.77 μmol, 12.31% yield, N/A purity) was obtained as a white solid. LCMS (Method D): Retention time: 0.414 min, [M+H]+=1343.7.

### Step 4: Synthesis of I-298

To a solution of Intermediate 1-6 (40.00 mg, 29.77 μmol, 1 eq) in DMF (0.4 mL) was added piperidine (5.07 mg, 59.54 μmol, 5.88 μL, 2 eq). The mixture was stirred at 25° C. for 1 hr. The reaction mixture was diluted with MeOH (1 ml). The residue was purified by prep-HPLC (column: CD02-Waters Xbidge BEH C18 150*25*10 um; mobile phase: [water (NH₄HCO₃)-ACN]; gradient: 40%-70% B over 10 min), and the eluent was concentrated to remove ACN and lyophilized to give product. I-298 (7.55 mg, 6.69 μmol, 22.48% yield, 99.396% purity) was obtained as a white solid. LCMS (Method H): Retention time: 0.683 min, [M+H]+=1121.7. SFC: Retention time: 3.451, 4.016 min. ¹H NMR (400 MHz, METHANOL-d₄) δ=8.37 (d, J=7.2 Hz, 1H), 7.99-7.92 (m, 1H), 7.91-7.80 (m, 2H), 7.76-7.66 (m, 2H), 7.55-7.41 (m, 3H), 7.40-7.35 (m, 1H), 7.16-7.14 (m, 2H), 6.58-6.40 (m, 2H), 5.24 (d, J=6.0 Hz, 1H), 4.64-4.55 (m, 3H), 4.39 (s, 2H), 4.10-4.02 (m, 2H), 3.84-3.71 (m, 11H), 3.69-3.44 (m, 8H), 3.25 (s, 1H), 3.19 (s, 1H), 3.16-3.06 (m, 1H), 2.96-2.86 (m, 2H), 2.80-2.65 (m, 2H), 2.56-2.36 (m, 5H), 2.28-2.19 (m, 2H), 2.12-2.01 (m, 3H), 1.91-1.75 (m, 10H), 1.64-1.52 (m, 2H), 1.46-1.40 (m, 3H), 1.26-1.19 (m, 1H). ¹⁹F NMR (377 MHz, METHANOL-d₄) δ=−120.802.

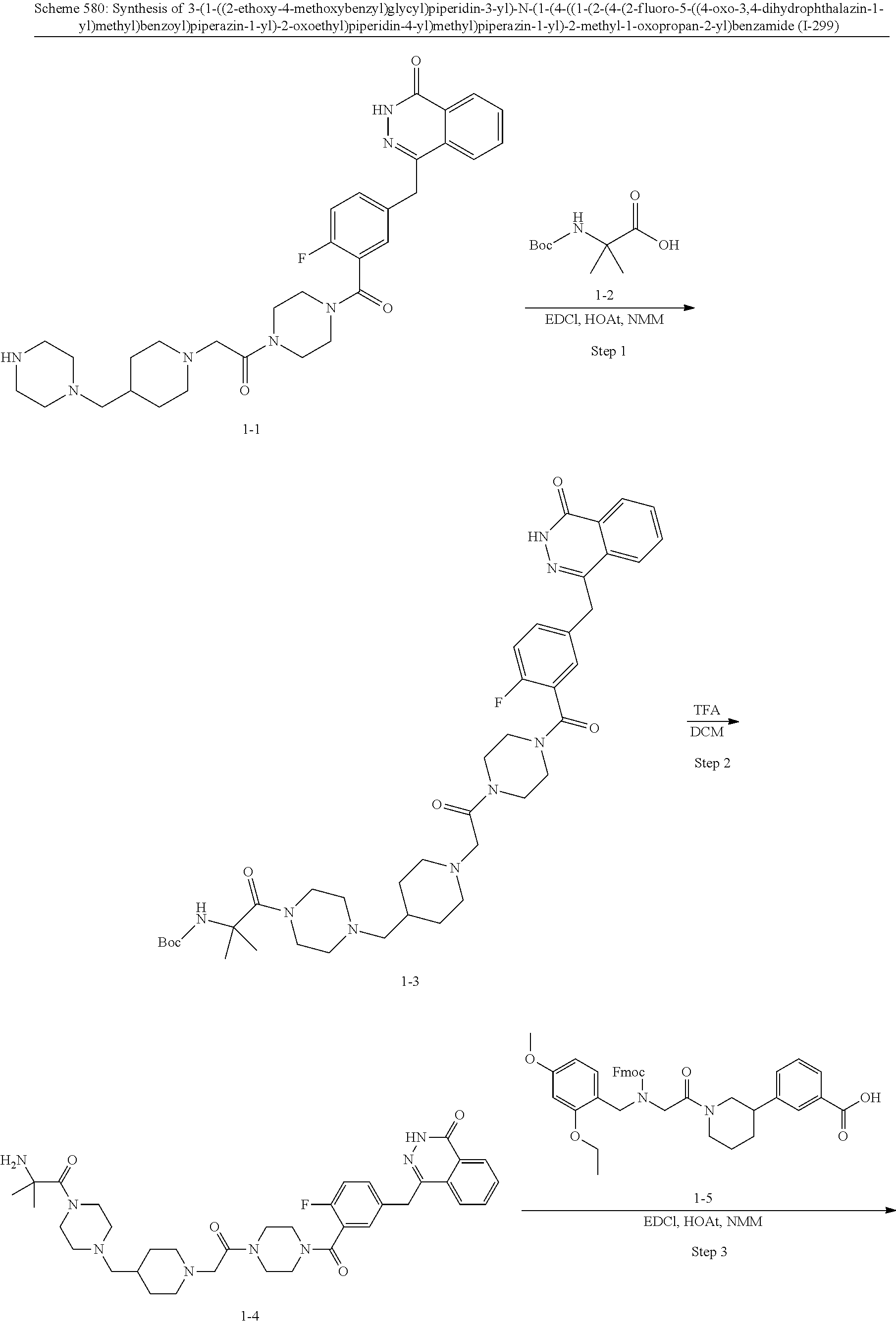

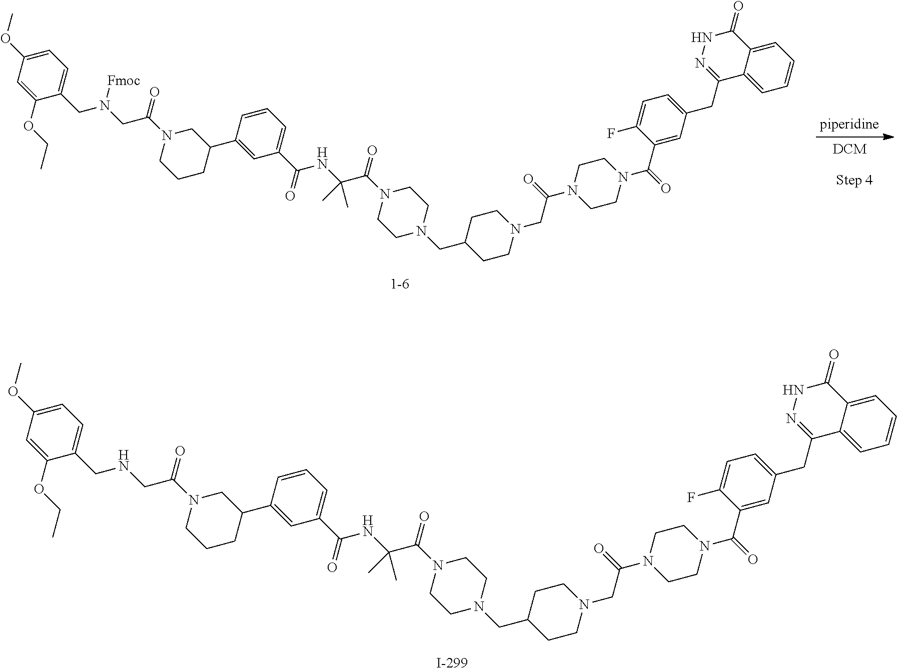

### Step 1: Synthesis of Intermediate 1-3

To a solution of Intermediate 1-1 (255.34 mg, 433.00 μmol, 1.1 eq) in DCM (3 mL) was added EDCI (226.38 mg, 1.18 mmol, 3 eq), HOAt (53.58 mg, 393.63 μmol, 55.06 μL, 1 eq) and NMM (199.08 mg, 1.97 mmol, 216.39 μL, 5 eq) and Intermediate 1-2 (80 mg, 393.63 μmol, 1 eq). The mixture was stirred at 25° C. for 2 hr. The reaction mixture was quenched by addition of H₂O (15 mL) at 25° C., and then the mixture was diluted with DCM (5 mL) and extracted with DCM (10 mL*3). The combined organic layers were washed with a saturated NaCl solution (20 mL), dried by Na₂SO₄, filtered and concentrated under reduced pressure to give Intermediate 1-3 (360 mg, crude) as a yellow oil. LCMS (Method E): Rt=0.275 min, [M+H]⁺=775.4.

### Step 2: Synthesis of Intermediate 1-4

To a solution of Intermediate 1-3 (340 mg, 438.75 μmol, 1 eq) in DCM (0.4 mL) was added TFA (153.50 mg, 1.35 mmol, 0.1 mL, 3.07 eq). The mixture was stirred at 25° C. for 2 hr. The mixture was concentrated under reduced pressure to give Intermediate 1-4 (330 mg, crude, TFA salt) as yellow oil. LCMS (Method E): Rt=0.278 min, [M+H]⁺=675.3.

### Step 3: Synthesis of Intermediate 1-6

To a solution of Intermediate 1-5 (262.72 mg, 404.97 μmol, 1.2 eq) in DCM (3 mL) was added NMM (170.67 mg, 1.69 mmol, 185.52 μL, 5 eq), HOAt (137.80 mg, 1.01 mmol, 141.63 μL, 3 eq), EDCI (64.70 mg, 337.48 μmol, 1 eq), and Intermediate 1-4 (300 mg, 337.48 μmol, 1 eq, TFA salt). The mixture was stirred at 25° C. for 2 hr. The reaction mixture was quenched by addition of H₂O (5 mL) at 25° C., and then diluted with DCM (5 mL) and extracted with DCM (5 mL*3). The combined organic layers were washed with a saturated NaCl solution (10 mL), dried by Na₂SO₄, filtered and concentrated under reduced pressure to give a residue. The residue was purified by prep-HPLC (neutral condition). The eluent was concentrated under reduced pressure to remove ACN and lyophilized to give Intermediate 1-6 (300 mg, 229.79 μmol, 68.09% yield) as a white solid. LCMS (Method E): Rt=0.423 min, [M+H]⁺=1305.4.

### Step 4: Synthesis of I-299

To a solution of Intermediate 1-6 (77 mg, 58.98 μmol, 1 eq) in DCM (2 mL) was added piperidine (86.22 mg, 1.01 mmol, 0.1 mL, 17.17 eq). The mixture was stirred at 25° C. for 2 hr. The mixture was concentrated under reduced pressure to give the residue. The residue was purified by prep-HPLC (column: CD07-Daisogel SP-100-8-ODS-PK 150*25*10 um; mobile phase: [water (NH₄HCO₃)-ACN]; gradient: 35%-65% B over 10 min). I-299 (17.58 mg, 15.96 μmol, 27.05% yield, 98.319% purity) was obtained as a white solid. LCMS (Method H): Rt=0.641 min, [M+H]⁺=1083.6. SFC: Rt=3.451 min, 4.178 min. ¹H NMR (400 MHz, METHANOL-d₄) δ=8.42-8.33 (m, 1H), 8.00-7.93 (m, 1H), 7.90-7.79 (m, 2H), 7.77-7.67 (m, 2H), 7.53-7.44 (m, 3H), 7.41-7.35 (m, 1H), 7.20-7.11 (m, 2H), 6.55-6.50 (m, 1H), 6.49-6.43 (m, 1H), 4.58-4.50 (m, 1H), 4.39 (s, 2H), 4.11-3.98 (m, 2H), 3.90-3.60 (m, 16H), 3.58-3.47 (m, 4H), 3.30-3.03 (m, 5H), 2.90-2.81 (m, 2H), 2.79-2.67 (m, 2H), 2.18-2.10 (m, 2H), 1.99 (br s, 4H), 1.79 (br s, 3H), 1.77-1.65 (m, 3H), 1.60-1.56 (m, 6H), 1.53-1.37 (m, 5H), 1.28-1.10 (m, 2H). ¹⁹F NMR (377 MHz, METHANOL-d₄) δ=−120.779.

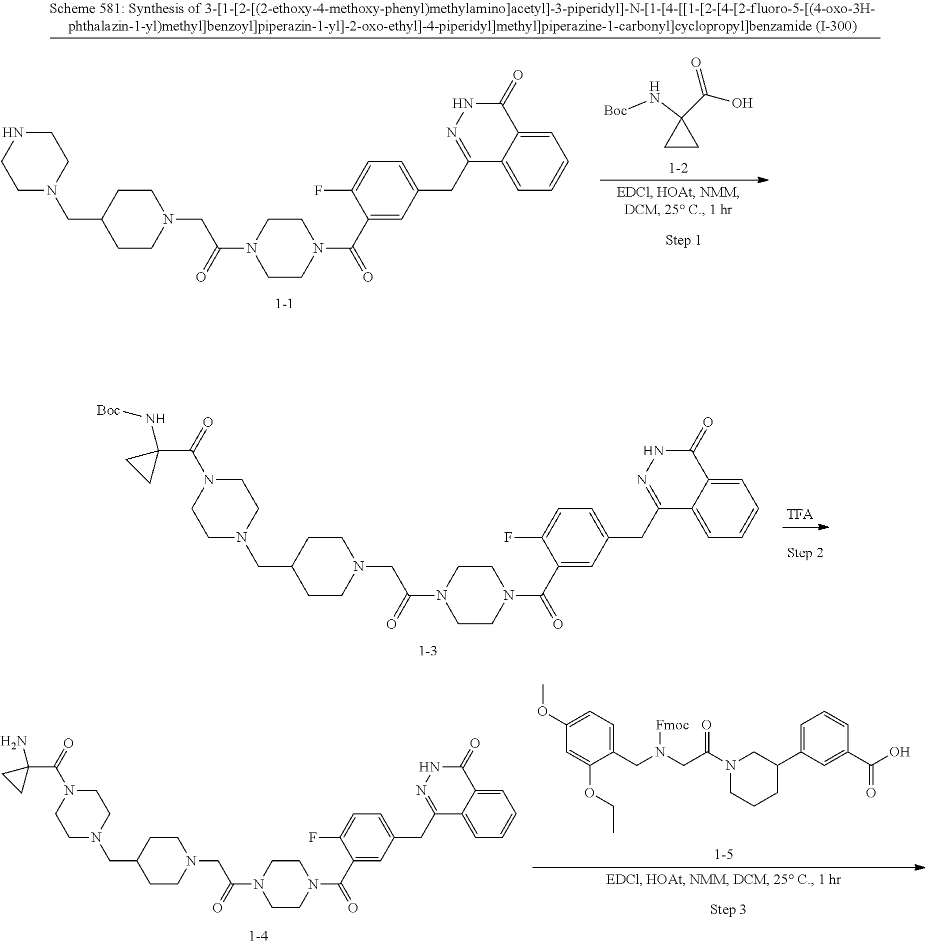

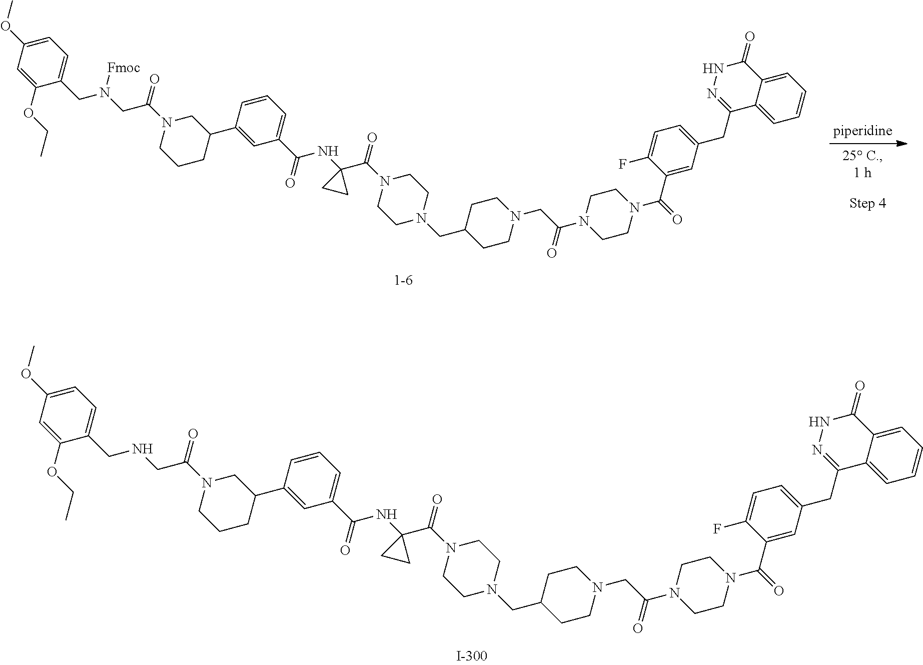

### Step 1: Synthesis of Intermediate 1-3

To a solution of Intermediate 1-2 (60 mg, 298.18 μmol, 1 eq) in DCM (2 mL) was added EDCI (171.49 mg, 894.55 μmol, 3 eq), HOAt (40.59 mg, 298.18 μmol, 41.71 μL, 1 eq) and NMM (150.80 mg, 1.49 mmol, 163.91 μL, 5 eq). Then Intermediate 1-1 (224.05 mg, 357.82 μmol, 1.2 eq, HCl salt) was added into the mixture and it was stirred at 25° C. for 1 hr. The reaction mixture was diluted with water (10 mL) and extracted with DCM (3 mL×3). The combined organic layers were washed with brine (3 mL×3) and dried over sodium sulfate, filtered and concentrated under reduced pressure to give a residue. The crude product was purified by reversed-phase HPLC (0.1% FA condition). The eluent was concentrated under reduced pressure to remove ACN and then lyophilized to give Intermediate 1-3 (180 mg, 230.32 μmol, 77.24% yield, 98.899% purity) as a yellow solid. LCMS (Method D): Rt: 0.276 min, [M+H]+=773.4. ¹H NMR (400 MHz, CHLOROFORM-d) δ=11.20-10.34 (m, 1H), 8.53-8.42 (m, 1H), 8.23 (s, 2H), 7.85-7.66 (m, 3H), 7.38-7.29 (m, 2H), 7.06-7.04 (m, 1H), 4.30 (s, 2H), 3.87-3.76 (m, 8H), 3.65 (s, 2H), 3.55-3.50 (m, 2H), 3.46 (s, 2H), 3.38-3.25 (m, 2H), 3.11-3.09 (m, 2H), 2.57-2.37 (m, 6H), 2.29-2.28 (m, 2H), 1.84-1.81 (m, 2H), 1.71-1.57 (m, 1H), 1.43 (s, 9H), 1.28-1.24 (m, 2H), 1.13-1.11 (m, 2H).

### Step 2: Synthesis of Intermediate 1-4

To a solution of Intermediate 1-3 (160 mg, 207.01 μmol, 1 eq) in DCM (2 mL) was added TFA (767.50 mg, 6.73 mmol, 500 μL, 32.52 eq). The mixture was stirred at 25° C. for 1 hr. The mixture was concentrated to give Intermediate 1-4 (160 mg, crude, TFA salt) as a brown oil. LCMS (Method D): Rt: 0.223 min, [M+H]+=673.2.

### Step 3: Synthesis of Intermediate 1-6

To a solution of Intermediate 1-5 (158.31 mg, 244.02 μmol, 1.2 eq) in DCM (2 mL) was added EDCI (116.95 mg, 610.06 μmol, 3 eq), HOAt (27.68 mg, 203.35 μmol, 28.45 μL, 1 eq) and NMM (102.84 mg, 1.02 mmol, 111.78 μL, 5 eq). The mixture was stirred at 25° C. for 0.5 hr. Then Intermediate 1-6 (160 mg, 203.35 μmol, 1 eq, TFA salt) was added into the mixture and it was stirred at 25° C. for 0.5 hr. The reaction mixture was diluted with water (12 mL) and extracted with DCM (4 mL×3). The combined organic layers were washed with brine (4 mL×3) and dried over sodium sulfate, filtered and concentrated under reduced pressure to give a residue. The crude product was purified by reversed-phase HPLC (H₂O). The eluent was concentrated under reduced pressure to remove ACN and then lyophilized to give Intermediate 1-6 (110 mg, 84.26 μmol, 41.43% yield, 99.845% purity) as a white solid. LCMS (Method H): Rt: 0.718 min, [M+H]+=1303.2. ¹H NMR (400 MHz, CHLOROFORM-d) δ=10.61-10.03 (m, 1H), 8.54-8.34 (m, 1H), 7.79-7.66 (m, 6H), 7.64-7.47 (m, 4H), 7.41-7.29 (m, 7H), 7.21-7.10 (m, 1H), 7.07-7.05 (m, 1H), 6.98-6.92 (m, 1H), 6.47-6.36 (m, 2H), 4.71-4.42 (m, 6H), 4.28 (s, 3H), 4.25-4.08 (m, 2H), 4.04-3.96 (m, 3H), 3.81 (d, J=6.4 Hz, 6H), 3.66-3.53 (m, 3H), 3.36-3.22 (m, 2H), 3.20-3.12 (m, 2H), 3.09-2.99 (m, 1H), 2.89-2.74 (m, 3H), 2.38 (s, 4H), 2.18-2.11 (m, 2H), 2.09-1.99 (m, 4H), 1.74 (d, J=10.0 Hz, 4H), 1.53-1.41 (m, 2H), 1.41-1.25 (m, 10H), 1.22-1.07 (m, 2H).

### Step 4: Synthesis of I-300

To a solution of Intermediate 1-6 (100 mg, 76.72 μmol, 1 eq) in THF (1 mL) was added piperidine (112.09 mg, 1.32 mmol, 130 μL, 17.16 eq). The mixture was stirred at 25° C. for 1 hr. The mixture was filtered to give a residue. The residue was purified by prep-HPLC (column: CD07-Daisogel SP-100-8-ODS-PK 150*25*10 um; mobile phase: [water (NH₄HCO₃)-ACN]; gradient: 26%-56% B over 10 min). The eluent was concentrated under reduced pressure to remove ACN and then lyophilized to give I-300 (27.37 mg, 25.10 μmol, 32.71% yield, 99.143% purity) as a white solid. LCMS (Method D): Rt: 0.301 min, [M+H]+=1081.5. ¹H NMR (400 MHz, METHANOL-d₄) δ=8.36 (d, J=8.0 Hz, 1H), 7.97-7.91 (m, 1H), 7.90-7.78 (m, 2H), 7.73-7.63 (m, 2H), 7.51-7.40 (m, 3H), 7.36 (s, 1H), 7.19-7.08 (m, 2H), 6.56-6.38 (m, 2H), 4.53 (d, J=9.2 Hz, 2H), 4.38 (s, 2H), 4.09-4.01 (m, 2H), 3.88-3.80 (m, 1H), 3.76 (s, 4H), 3.74-3.69 (m, 4H), 3.66 (s, 4H), 3.54 (s, 1H), 3.52-3.43 (m, 3H), 3.29-3.25 (m, 1H), 3.22-3.14 (m, 2H), 2.94 (d, J=2.4 Hz, 1H), 2.91-2.80 (m, 2H), 2.76-2.63 (m, 2H), 2.36 (s, 4H), 2.14-2.13 (m, 2H), 2.07-1.98 (m, 3H), 1.88-1.77 (m, 2H), 1.73-1.63 (m, 3H), 1.56-1.50 (m, 1H), 1.48-1.35 (m, 6H), 1.31-1.16 (m, 2H), 1.12 (s, 2H). ¹⁹F NMR (377 MHz, METHAN OL-d₄) δ=−120.794. SFC: Rt: 3.022, 3.268 min.

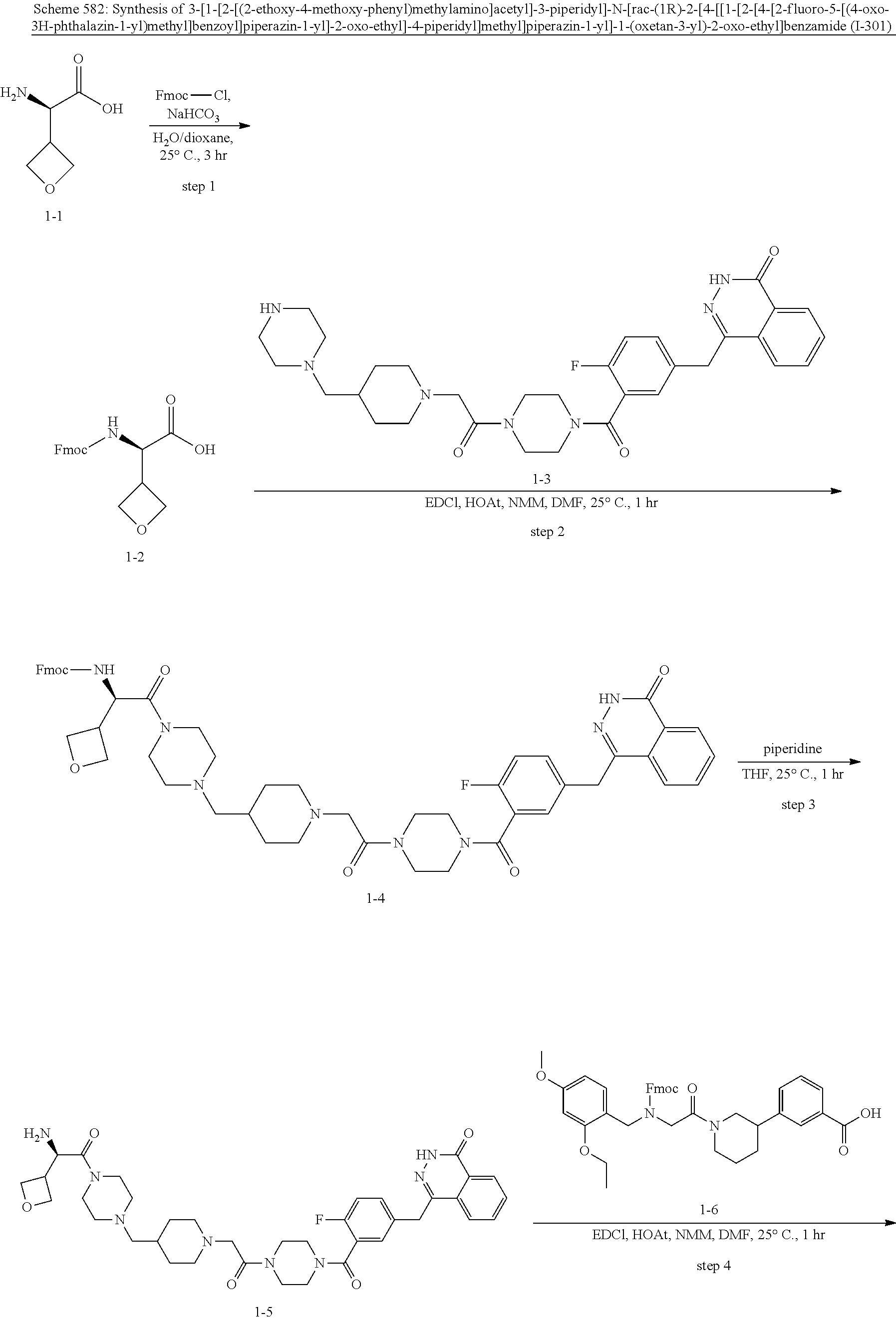

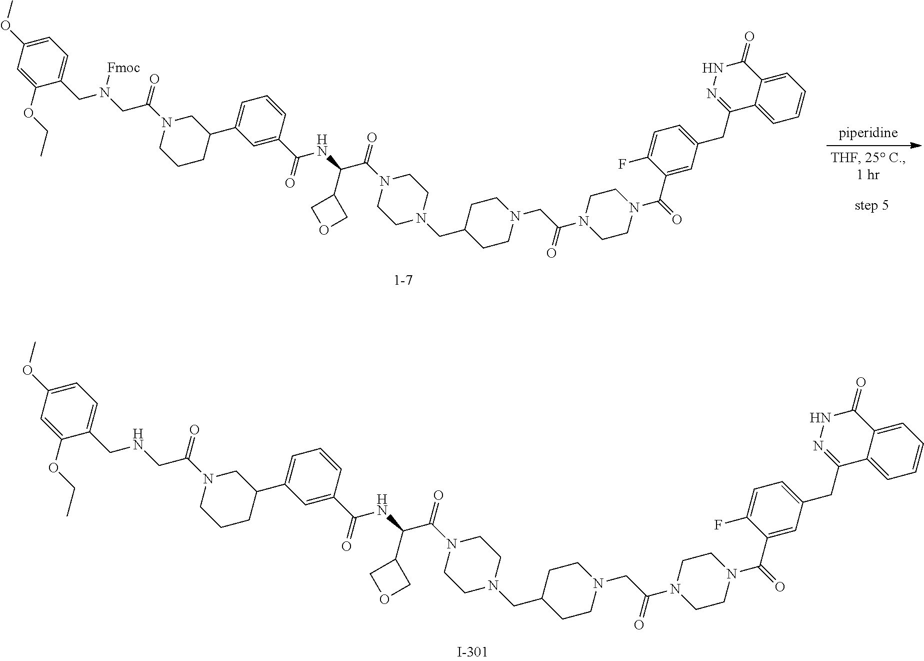

### Step 1: Synthesis of Intermediate 1-2

To a solution of Intermediate 1-1 (700 mg, 5.34 mmol, 1 eq) in dioxane (3.5 mL) and H₂O (3.5 mL) was added Fmoc-Cl (1.66 g, 6.41 mmol, 1.2 eq) and NaHCO₃ (1.35 g, 16.01 mmol, 623.13 μL, 3 eq). The mixture was stirred at 25° C. for 3 hr. The reaction mixture was concentrated in vacuo. The crude product was used in the next step without further purification. Intermediate 1-2 (1.8 g, crude) was obtained as a white gum. LCMS (Method F): Rt=0.338 min, [M+H]+=354.0.

### Step 2: Synthesis of Intermediate 1-4

To a solution of Intermediate 1-2 (500 mg, 1.41 mmol, 1 eq) and Intermediate 1-3 (885.99 mg, 1.41 mmol, 1 eq, HCl salt) in DMF (8 mL) was added EDCI (813.75 mg, 4.24 mmol, 3 eq), HOAt (192.59 mg, 1.41 mmol, 197.94 μL, 1 eq) and NMM (715.59 mg, 7.07 mmol, 777.82 μL, 5 eq). The mixture was stirred at 25° C. for 1 hr. The mixture was washed with water (40 mL) and extracted with EA (45 mL*3). The combined organic layers were dried over sodium sulfate, filtered and concentrated under reduced pressure to give a residue. The residue was purified by flash silica gel chromatography (ISCO; 40 g SepaFlash Silica Flash Column, Eluent of 0˜100% MeOH/EA @80 mL/min) and then the eluent was concentrated in vacuum to give a product. Intermediate 1-4 (900 mg, 856.17 μmol, 60.51% yield, 88% purity) was obtained as a yellow solid. LCMS (Method D): Rt=0.342 min, [M+H]+=925.4. ¹H NMR (400 MHz, METHANOL-d4) δ=8.36 (d, J=7.6 Hz, 1H), 7.96-7.92 (m, 1H), 7.86-7.78 (m, 3H), 7.73-7.68 (m, 1H), 7.66 (d, J=7.2 Hz, 2H), 7.48 (d, J=3.2 Hz, 1H), 7.39-7.36 (m, 3H), 7.31 (d, J=7.4 Hz, 2H), 7.16-7.15 (m, 1H), 4.77-4.68 (m, 3H), 4.61-4.55 (m, 2H), 4.49-4.40 (m, 2H), 4.38 (s, 2H), 4.21-4.19 (m, 1H), 3.81-3.61 (m, 6H), 3.58-3.48 (m, 4H), 3.42-3.41 (m, 2H), 3.30-3.11 (m, 3H), 3.00-2.86 (m, 2H), 2.49-2.31 (m, 3H), 2.21-2.08 (m, 4H), 1.74 (d, J=1.2 Hz, 2H), 1.65-1.46 (m, 2H), 1.33-1.25 (m, 2H). ¹⁹F NMR (376 MHz, METHANOL-d4) δ=−120.708.

### Step 3: Synthesis of Intermediate 1-5

To a solution of Intermediate 1-4 (800 mg, 864.81 μmol, 1 eq) in THF (8 mL) was added piperidine (862.20 mg, 10.13 mmol, 1 mL). The mixture was stirred at 25° C. for 1 hr. The reaction mixture was concentrated in vacuum. The residue was purified by prep-HPLC (basic condition), the eluent was concentrated under reduced pressure to remove ACN and lyophilized to give product. Intermediate 1-5 (390 mg, 521.62 μmol, 60.32% yield, 94% purity) was obtained as a yellow solid. LCMS (Method D): Rt=0.220 min, [M+H]+=703.4. SFC: Rt=0.926 min. ¹H NMR (400 MHz, METHANOL-d4) δ=8.37 (d, J=7.6 Hz, 1H), 7.94 (s, 1H), 7.90-7.80 (m, 2H), 7.48 (s, 1H), 7.38 (s, 1H), 7.16-7.14 (m, 1H), 4.72-4.71 (m, 3H), 4.64-4.60 (m, 1H), 4.44-4.42 (m, 1H), 4.38 (s, 2H), 4.09 (d, J=7.6 Hz, 1H), 3.82-3.64 (m, 6H), 3.57 (s, 3H), 3.52 (d, J=3.6 Hz, 1H), 3.26-3.15 (m, 3H), 2.97-2.84 (m, 3H), 2.50 (s, 1H), 2.45-2.35 (m, 3H), 2.22-2.21 (m, 2H), 2.06-2.04 (m, 2H), 1.76-1.73 (m, 2H), 1.65 (s, 1H), 1.55 (s, 1H), 1.31-1.16 (m, 2H). ¹⁹F NMR (376 MHz, METHANOL-d4) δ=−120.771.

### Step 4: Synthesis of Intermediate 1-7

To a solution of Intermediate 1-5 (100 mg, 142.28 μmol, 1 eq) and Intermediate 1-6 (92.31 mg, 142.28 μmol, 1 eq) in DMF (1 mL) was added EDCI (81.83 mg, 426.85 μmol, 3 eq), HOAt (19.37 mg, 142.28 μmol, 19.90 μL, 1 eq) and NMM (71.96 mg, 711.42 μmol, 78.22 μL, 5 eq). The mixture was stirred at 25° C. for 1 hr. The reaction mixture was concentrated in vacuum. The residue was purified by prep-HPLC (0.1% FA condition), the eluent was concentrated under reduced pressure to remove ACN and lyophilized to give product. Intermediate 1-7 (100 mg, 70.49 μmol, 49.54% yield, 94% purity) was obtained as a white solid. LCMS (Method D): Rt=0.458 min, [M+H]+=1334.7.

### Step 5: Synthesis of I-301

To a solution of Intermediate 1-7 (90 mg, 67.49 μmol, 1 eq) in THF (8 mL) was added piperidine (862.20 mg, 10.13 mmol, 1 mL, 150.04 eq). The mixture was stirred at 25° C. for 1 hr. The reaction mixture was concentrated in vacuum. The residue was purified by reversed phase chromatography (0.1% NH₃·H₂O condition), the eluent was concentrated under reduced pressure to remove ACN and lyophilized to give product. I-301 (11.88 mg, 10.69 μmol, 15.84% yield, 100% purity) was obtained as a white solid. LCMS (Method D): Rt=0.309 min, [M+H]+=1111.7. SFC: Rt=3.529 min, 3.941 min, 6.214 min, 6.745 min. ¹H NMR (400 MHz, METHANOL-d4) δ=8.40-8.33 (m, 1H), 7.97-7.92 (m, 1H), 7.89-7.81 (m, 2H), 7.78-7.70 (m, 2H), 7.50-7.42 (m, 3H), 7.37 (s, 1H), 7.18-7.11 (m, 2H), 6.53-6.44 (m, 2H), 5.53-5.41 (m, 1H), 4.81-4.77 (m, 2H), 4.67-4.65 (m, 1H), 4.59-4.49 (m, 2H), 4.44-4.40 (m, 1H), 4.38 (s, 2H), 4.10-4.02 (m, 2H), 3.88-3.78 (m, 2H), 3.78-3.75 (m, 5H), 3.73 (d, J=6.0 Hz, 3H), 3.65 (s, 3H), 3.61-3.53 (m, 3H), 3.50 (d, J=6.0 Hz, 3H), 3.45-3.37 (m, 1H), 3.24-3.16 (m, 2H), 3.14-3.05 (m, 1H), 2.91-2.83 (m, 2H), 2.78-2.65 (m, 2H), 2.45 (s, 2H), 2.36 (d, J=2.0 Hz, 2H), 2.18-2.17 (m, 2H), 2.08-1.99 (m, 3H), 1.84-1.83 (m, 2H), 1.77-1.69 (m, 2H), 1.62-1.50 (m, 2H), 1.46-1.38 (m, 3H), 1.29-1.15 (m, 2H). ¹⁹F NMR (376 MHz, METHANOL-d4) δ=−120.787.

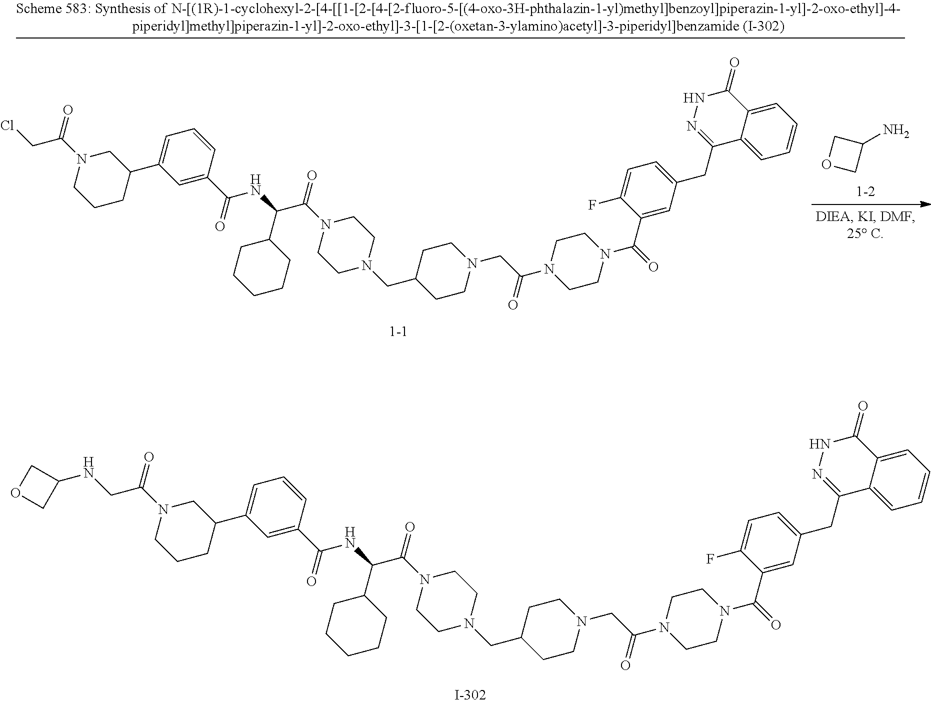

To the solution of Intermediate 1-1 (150 mg, 151.12 μmol, 1 eq) in DMF (1.5 mL) was added Intermediate 1-2 (16.57 mg, 226.67 μmol, 1.5 eq) and DIEA (78.12 mg, 604.46 μmol, 105.28 μL, 4 eq) and KI (100.34 mg, 604.46 μmol, 4 eq), the mixture was stirred at 25° C. for 12 hr. The mixture was concentrated under vacuum. The residue was purified by Prep-HPLC (column: Waters Xbridge 150*25 mm*5 um; mobile phase: [water (NH₄HCO₃)-ACN]; gradient: 30%-60% B over 9 min) and dried by lyophilization to give I-302 (43.4 mg, 42.14 μmol, 27.88% yield, 99.928% purity) as a white solid. LCMS (Method G): Rt=0.583 min, [M+H]⁺=1029.7. SFC: Rt=5.687, 6.814 min. ¹H NMR (400 MHz, CHLOROFORM-d) δ=10.40-10.00 (m, 1H), 8.53-8.40 (m, 1H), 7.81-7.75 (m, 2H), 7.75-7.65 (m, 3H), 7.46-7.29 (m, 4H), 7.08-7.04 (m, 2H), 5.12-4.99 (m, 1H), 4.85-4.75 (m, 2H), 4.75-4.61 (m, 1H), 4.58-4.48 (m, 2H), 4.29 (s, 2H), 4.07-3.94 (m, 1H), 3.87-3.54 (m, 11H), 3.50-3.18 (m, 6H), 3.13-3.02 (m, 1H), 2.93-2.82 (m, 2H), 2.79-2.57 (m, 2H), 2.46 (br s, 2H), 2.41 (br s, 2H), 2.21 (br d, J=6.4 Hz, 2H), 2.16-2.03 (m, 3H), 1.91-1.83 (m, 2H), 1.71-1.65 (m, 5H), 1.64-1.45 (m, 4H), 1.34-1.04 (m, 8H).

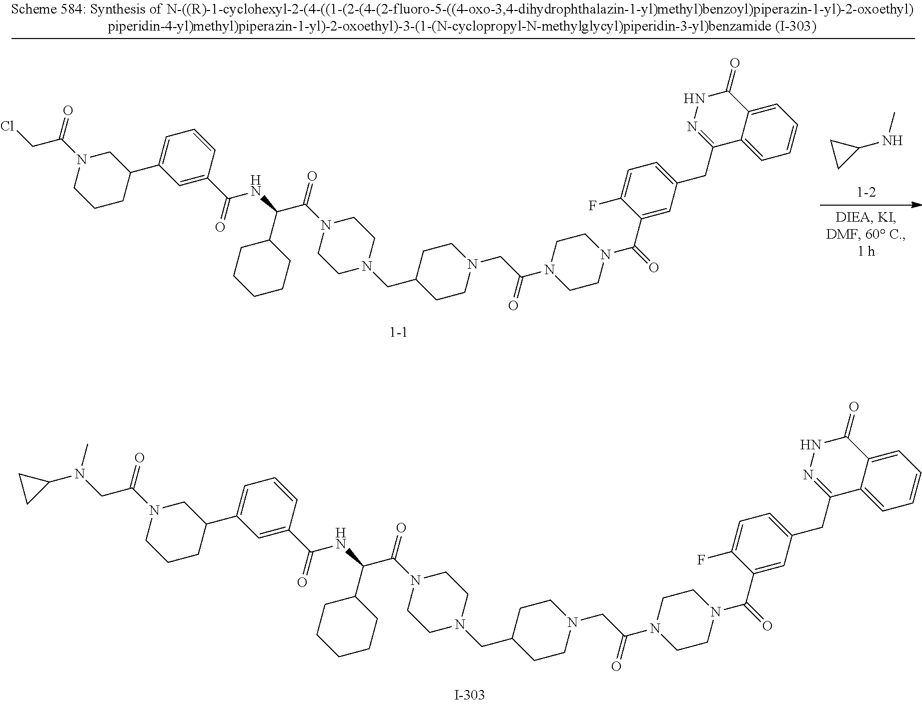

To a solution of Intermediate 1-1 (50 mg, 50.37 μmol, 1 eq) and Intermediate 1-2 (5.37 mg, 75.56 μmol, 1.5 eq) in DMF (1 mL) was added KI (33.45 mg, 201.49 μmol, 4 eq) and DIEA (26.04 mg, 201.49 μmol, 35.09 μL, 4 eq). The mixture was stirred at 60° C. for 1 hr. The reaction was filtered and concentrated under reduced pressure to give a residue. The residue was purified by prep-HPLC (column: YMC-Actus Triart C18 150*30 mm*7 um; mobile phase: [water (FA)-ACN]; gradient: 10%-40% B over 10 min). The eluent was concentrated under reduced pressure to remove ACN, then lyophilized to afford I-303 (33 mg, 30.05 μmol, 59.65% yield, 93.53% purity) as a white solid. LCMS (Method E): Rt=0.422 min, [M+H]⁺=1027.6. SFC: Rt=4.441 min, 5.819 min. ¹H NMR (400 MHz, METHANOL-d₄) δ=8.37 (d, J=7.2 Hz, 1H), 8.00-7.69 (m, 5H), 7.59-7.42 (m, 3H), 7.42-7.34 (m, 1H), 7.20-7.15 (m, 1H), 4.64-4.51 (m, 2H), 4.48-4.24 (m, 6H), 3.91-3.76 (m, 5H), 3.73 (br s, 1H), 3.70-3.61 (m, 2H), 3.56 (d, J=5.2 Hz, 3H), 3.39 (br d, J=0.8 Hz, 2H), 3.10 (br s, 2H), 3.06-2.98 (m, 3H), 2.96-2.86 (m, 2H), 2.85-2.46 (m, 7H), 2.18-1.85 (m, 9H), 1.83-1.66 (m, 5H), 1.65-1.53 (m, 2H), 1.51-1.45 (m, 1H), 1.39-1.20 (m, 4H), 1.18-1.01 (m, 4H), 0.99-0.81 (m, 2H).

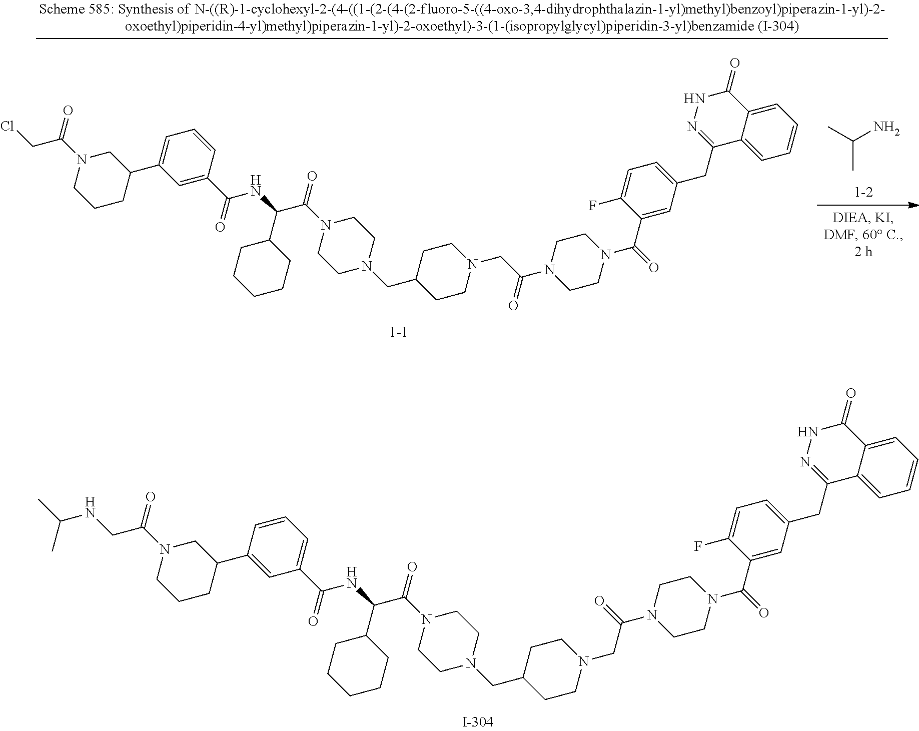

To a solution of Intermediate 1-1 (50 mg, 50.37 μmol, 1 eq) and Intermediate 1-2 (4.47 mg, 75.56 μmol, 6.49 μL, 1.5 eq) in DMF (1 mL) was added KI (33.45 mg, 201.49 μmol, 4 eq) and DIEA (26.04 mg, 201.49 μmol, 35.09 μL, 4 eq). The mixture was stirred at 60° C. for 2 hr. The reaction was filtered to give a residue. The residue was purified by prep-HPLC (column: YMC-Actus Triart C18 150*30 mm*7 um; mobile phase: [water (FA)-ACN]; gradient: 10%-40% B over 10 min). The eluent was concentrated under reduced pressure to remove ACN, then lyophilized to afford product. The product was further purified by prep-HPLC (column: Waters Xbridge 150*25 mm*5 um; mobile phase: [water (NH₄HCO₃)-ACN]; gradient: 48%-78% B over 9 min). The eluent was concentrated under reduced pressure to remove ACN, then lyophilized to afford I-304 (15 mg, 14.77 μmol, 29.33% yield, 100% purity) as a white solid. LCMS (Method E): Rt=0.408 min, M+H=1015.9. SFC: Rt=2.119 min, 2.335 min. ¹H NMR (400 MHz, METHANOL-d₄) δ=8.40-8.34 (m, 1H), 7.99-7.93 (m, 1H), 7.91-7.80 (m, 2H), 7.76 (br s, 1H), 7.71-7.69 (m, 1H), 7.54-7.34 (m, 4H), 7.19-7.16 (m, 1H), 4.67-4.52 (m, 2H), 4.39 (s, 2H), 3.96-3.76 (m, 4H), 3.75-3.64 (m, 4H), 3.60-3.39 (m, 6H), 3.26-3.09 (m, 3H), 2.93-2.71 (m, 5H), 2.55-2.32 (m, 4H), 2.27-2.17 (m, 2H), 2.14-2.01 (m, 3H), 1.97-1.83 (m, 4H), 1.82-1.62 (m, 7H), 1.61-1.51 (m, 1H), 1.37-1.27 (m, 4H), 1.27-1.20 (m, 2H), 1.17-1.04 (m, 8H).

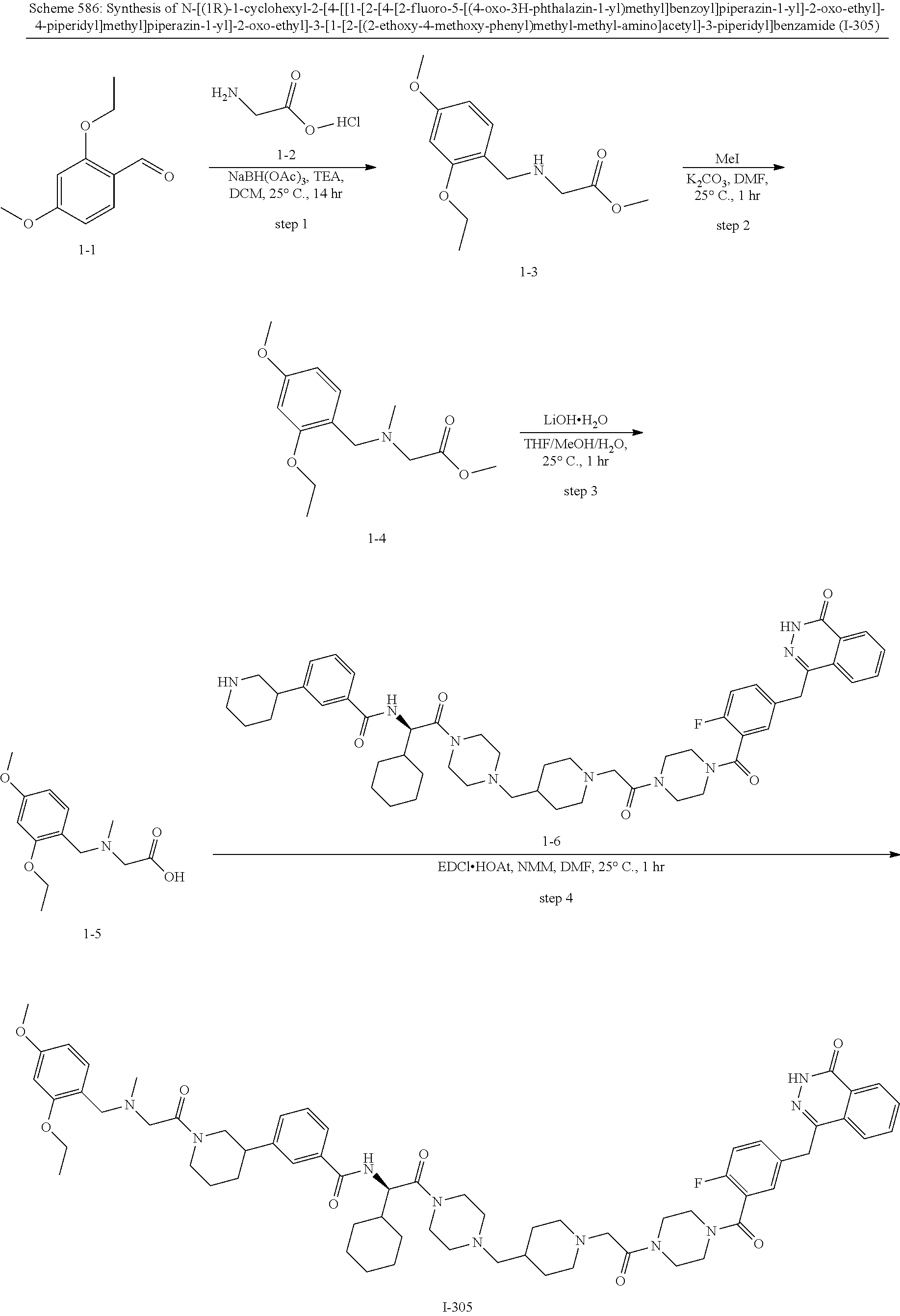

### Step 1: Synthesis of Intermediate 1-2

To a solution of Intermediate 1-1 (1 g, 5.55 mmol, 1 eq) and Intermediate 1-2 (1.39 g, 11.10 mmol, 2 eq) in DCM (10 mL) was added TEA (1.12 g, 11.10 mmol, 1.54 mL, 2 eq). The mixture was stirred at 25° C. for 2 hr. Then NaBH(OAc)₃ (1.76 g, 8.32 mmol, 1.5 eq) was added to the mixture and the mixture was stirred at 25° C. for 12 h. The mixture was washed with water (10 mL) and extracted with DCM (15 mL*3). The combined organic layers were dried over sodium sulfate, filtered and concentrated under reduced pressure to give a residue. The crude product was used in the next step without further purification. Intermediate 1-3 (1.4 g, 4.86 mmol, 87.65% yield, 88% purity) was obtained as a yellow oil. LCMS (Method D): Rt=0.216 min, [M−88]⁺=165.2. ¹H NMR (400 MHz, DMSO-d₆) δ=7.14 (d, J=8.4 Hz, 1H), 6.50 (d, J=2.4 Hz, 1H), 6.45-6.43 (m, 1H), 4.00-3.97 (m, 2H), 3.72 (s, 3H), 3.62-3.59 (m, 5H), 3.29 (s, 2H), 1.32-1.30 (m, 3H).

### Step 2: Synthesis of Intermediate 1-4

To a solution of Intermediate 1-3 (500 mg, 1.97 mmol, 1 eq) in DMF (5 mL) was added K₂CO₃ (818.45 mg, 5.92 mmol, 3 eq) and MeI (336.22 mg, 2.37 mmol, 147.47 μL, 1.2 eq). The mixture was stirred at 25° C. for 1 hr. The mixture was washed with water (5 mL) and extracted with EA (10 mL*3). The combined organic layers were dried over sodium sulfate, filtered and concentrated under reduced pressure to give a residue. The residue was purified by flash silica gel chromatography (ISCO; 20 g SepaFlash Silica Flash Column, Eluent of 0-100% EA/PE @40 mL/min) and then the eluent was concentrated in vacuum to give product. Intermediate 1-4 (150 mg, 521.85 μmol, 26.44% yield, 93% purity) was obtained as a yellow oil. LCMS (Method F): Rt=0.573 min, [M+H]⁺=268.1. ¹H NMR (400 MHz, METHANOL-d4) δ=7.17 (d, J=8.0 Hz, 1H), 6.51-6.46 (m, 2H), 4.02-4.00 (m, 2H), 3.78 (s, 3H), 3.69 (s, 3H), 3.66 (s, 2H), 3.23 (s, 2H), 2.33 (s, 3H), 1.40-1.37 (m, 3H).

### Step 3: Synthesis of Intermediate 1-5

To a solution of Intermediate 1-4 (140 mg, 523.72 μmol, 1 eq) in THF (0.5 mL), MeOH (0.5 mL) and H₂O (0.5 mL) was added LiOH·H₂O (65.93 mg, 1.57 mmol, 3 eq). The mixture was stirred at 25° C. for 1 hr. The pH was acidified to 5-6 with 1N HCl, and the mixture was diluted with EA, washed with water (5 mL) and extract with EA (8 mL*3). The combined organic layers were dried over sodium sulfate, filtered and concentrated under reduced pressure to give a residue. The crude product was used in the next step without further purification. Intermediate 1-5 (60 mg, 234.51 μmol, 44.78% yield, 99% purity) was obtained as yellow gum. LCMS (Method F): Rt=0.329 min, [M+H]⁺=254.0.

### Step 4: Synthesis of I-305

To a solution of Intermediate 1-5 (13.82 mg, 54.58 μmol, 1 eq) and Intermediate 1-6 (50 mg, 54.58 μmol, 1 eq) in DMF (1 mL) was added EDCI (31.39 mg, 163.73 μmol, 3 eq), HOAt (7.43 mg, 54.58 μmol, 7.63 μL, 1 eq) and NMM (27.60 mg, 272.89 μmol, 30.00 μL, 5 eq). The mixture was stirred at 25° C. for 1 hr. The resulting mixture was filtered, and the filtrate was concentrated under reduced pressure. The residue was purified by prep-HPLC (0.1% FA condition), the eluent was concentrated under reduced pressure to remove ACN and lyophilized to give product. I-305 (23.41 mg, 20.28 μmol, 37.15% yield, 99.735% purity) was obtained as a white solid. LCMS (Method D): Rt=0.353 min, [M+H]⁺=1151.7. SFC: Rt=3.522 min, 4.097 min. ¹H NMR (400 MHz, DMSO-d₆) δ=12.36 (s, 1H), 8.28 (d, J=7.2 Hz, 1H), 8.12 (d, J=8.8 Hz, 1H), 7.97-7.92 (m, 1H), 7.89-7.80 (m, 2H), 7.77-7.72 (m, 2H), 7.44-7.40 (m, 1H), 7.40-7.33 (m, 3H), 7.20-7.19 (m, 1H), 7.16-7.03 (m, 1H), 6.55-6.36 (m, 2H), 5.60 (d, J=1.6 Hz, 1H), 4.81-4.80 (m, 1H), 4.33 (s, 2H), 4.01-3.88 (m, 2H), 3.72 (s, 4H), 3.56 (s, 4H), 3.46 (s, 3H), 2.99 (s, 1H), 2.77 (d, J=10.8 Hz, 2H), 2.33 (d, J=1.6 Hz, 4H), 2.22-2.20 (m, 2H), 2.18 (s, 3H), 2.14 (s, 4H), 2.12 (s, 1H), 2.05-1.87 (m, 5H), 1.80-1.58 (m, 11H), 1.51-1.50 (m, 3H), 1.34-1.24 (m, 4H), 1.21-1.14 (m, 3H), 1.14-1.04 (m, 4H), 1.00-0.98 (m, 3H). ¹⁹F NMR (376 MHz, DMSO-d₆) δ=−119.765.

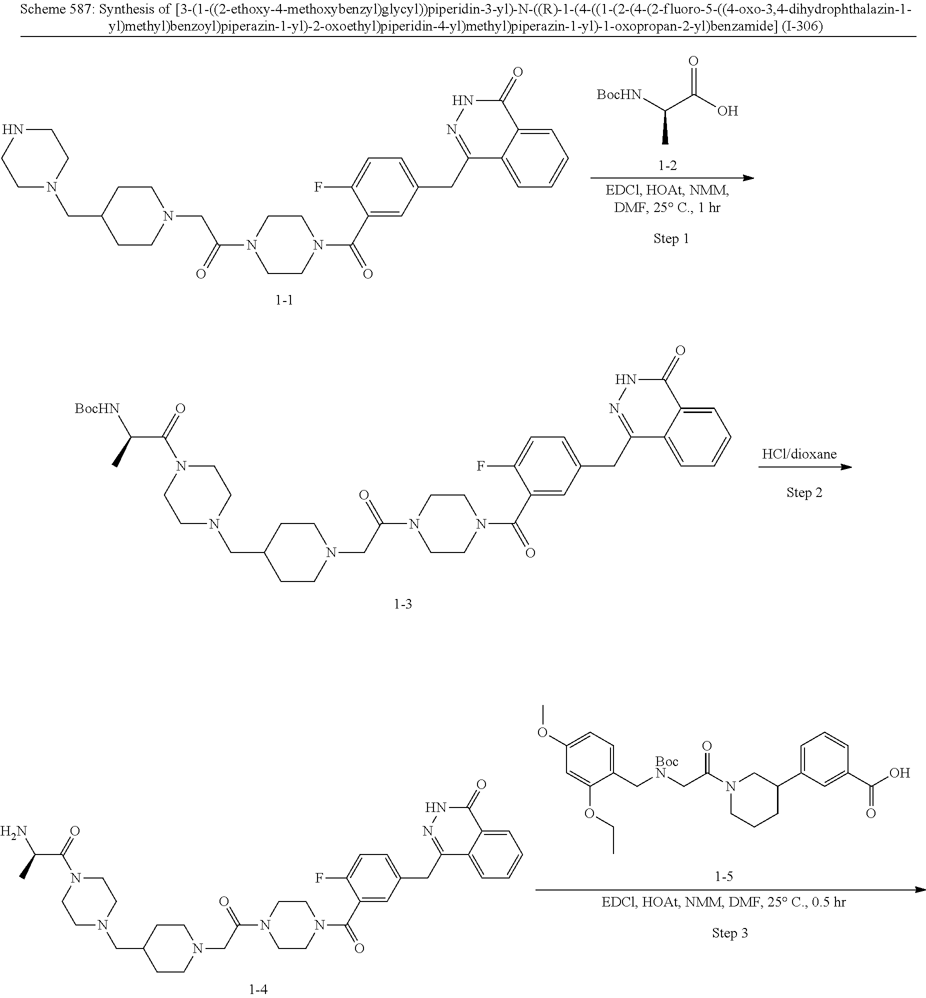 [SMILES: O=C(CN1CCC(CN2CCNCC2)CC1)N1CCN(C(=O)c2cc(Cc3n[nH]c(=O)c4ccccc34)ccc2F)CC1 ; C[C@@H](NC(=O)OC(C)(C)C)C(=O)O ; C[C@@H](NC(=O)OC(C)(C)C)C(=O)N1CCN(CC2CCN(CC(=O)N3CCN(C(=O)c4cc(Cc5n[nH]c(=O)c6ccccc56)ccc4F)CC3)CC2)CC1 ; C[C@@H](N)C(=O)N1CCN(CC2CCN(CC(=O)N3CCN(C(=O)c4cc(Cc5n[nH]c(=O)c6ccccc56)ccc4F)CC3)CC2)CC1 ; CCOc1cc(OC)ccc1CN(CC(=O)N1CCCC(c2cccc(C(=O)O)c2)C1)C(=O)OC(C)(C)C]

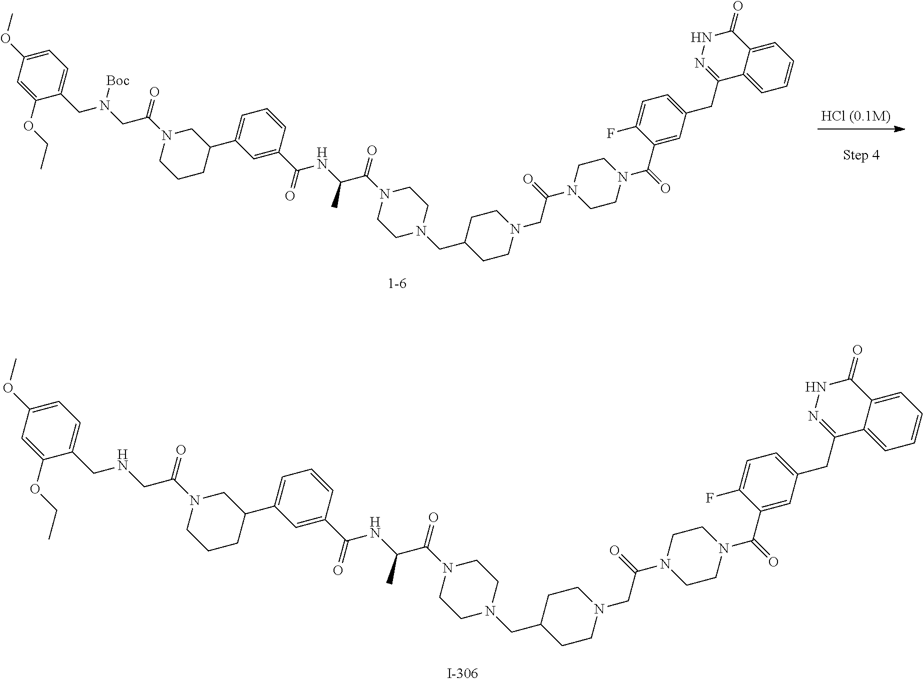 [SMILES: CCOc1cc(OC)ccc1CN(CC(=O)N1CCCC(c2cccc(C(=O)N[C@H](C)C(=O)N3CCN(CC4CCN(CC(=O)N5CCN(C(=O)c6cc(Cc7n[nH]c(=O)c8ccccc78)ccc6F)CC5)CC4)CC3)c2)C1)C(=O)OC(C)(C)C ; CCOc1cc(OC)ccc1CNCC(=O)N1CCCC(c2cccc(C(=O)N[C@H](C)C(=O)N3CCN(CC4CCN(CC(=O)N5CCN(C(=O)c6cc(Cc7n[nH]c(=O)c8ccccc78)ccc6F)CC5)CC4)CC3)c2)C1]

### Step 1. Synthesis of Intermediate 1-3

To a solution of Intermediate 1-1 (100 mg, 169.58 gmol, 1 eq) and Intermediate 1-2 (35.29 mg, 186.53 μmol, 1.1 eq) in DMF (1 mL) was added EDCI (97.52 mg, 508.73 μmol, 3 eq), NMM (85.76 mg, 847.89 μmol, 93.22 μL, 5 eq), and HOAt (23.08 mg, 169.58 μmol, 23.72 μL, 1 eq). The mixture was stirred at 25° C. for 1 hr. The reaction mixture was diluted with H₂O (3 mL) and extracted with EA (5 mL*2). The combined organic layers were washed with NaCl (aq) (3 mL*2), dried over Na₂SO₄, filtered and concentrated under reduced pressure to give a residue. The residue was purified by prep-HPLC (0.1% FA) the eluent was concentrated to remove ACN and lyophilized to give Intermediate 1-3 (60 mg, 78.85 μmol, 46.50% yield) as a white solid. LCMS (Method D): Retention time: 0.271 min, [M+H]+=761.1.

### Step 2: Synthesis of Intermediate 1-4

A solution of Intermediate 1-3 (50 mg, 65.71 μmol, 1 eq) in HCl/dioxane (0.1 mL) and DCM (0.1 mL) was stirred at 25° C. for 0.5 hr. The reaction mixture was concentrated under reduced pressure to give Intermediate 1-4 (50 mg, crude, HCl salt) as a white solid. LCMS (Method D): Retention time: 0.537 min, [M+H]+=661.4.

### Step 3: Synthesis of Intermediate 1-6

To a solution of Intermediate 1-4 (50 mg, 71.71 μmol, 1 eq, HCl salt) and Intermediate 1-5 (45.32 mg, 86.05 μmol, 1.2 eq) in DMF (1 mL) was added NMM (36.27 mg, 358.56 μmol, 39.42 μL, 5 eq), HOAt (9.76 mg, 71.71 μmol, 10.03 μL, 1 eq), EDCI (41.24 mg, 215.13 μmol, 3 eq). The mixture was stirred at 25° C. for 0.5 hr. The reaction mixture was diluted with H₂O (6 mL) and extracted with EA (5 mL*2). The combined organic layers were washed with NaCl (aq) (5 mL*2), dried over Na₂SO₄, filtered and concentrated under reduced pressure to give a residue. The residue was purified by prep-HPLC (0.1% FA) the eluent was concentrated to remove ACN and lyophilized to give Intermediate 1-6 (65 mg, 55.58 μmol, 77.51% yield) as a white solid. LCMS (Method D): Retention time: 0.420 min, [M/2+H]+=585.5. SFC: Retention time: 0.944 min.

### Step 4: Synthesis of I-306

A solution of Intermediate 1-6 (20 mg, 17.10 μmol, 1 eq) in HCl (0.1 M, 171.03 μL, 1 eq) was stirred at 100° C. for 0.5 hr. The reaction mixture was concentrated under reduced pressure to give a residue. The residue was purified by prep-HPLC (0.1% FA) the eluent was concentrated to remove ACN and lyophilized to give product. I-306 (11.69 mg, 10.14 μmol, 59.26% yield, 92.706% purity) was obtained as a white solid. LCMS (Method D): Retention time: 0.354 min, [M+H]+=1069.6. SFC: Retention time: 4.507 min, 6.182 min. ¹H NMR (400 MHz, METHANOL-d₄) δ=8.37 (d, J=7.6 Hz, 1H), 7.99-7.93 (m, 1H), 7.91-7.73 (m, 4H), 7.55-7.43 (m, 3H), 7.41-7.34 (m, 1H), 7.33-7.27 (m, 1H), 7.20-7.15 (m, 1H), 6.66-6.61 (m, 1H), 6.61-6.54 (m, 1H), 5.09-5.00 (m, 1H), 4.89 (d, J=4.8 Hz, 1H), 4.61-4.51 (m, 1H), 4.39 (s, 2H), 4.35-4.31 (m, 1H), 4.27 (s, 1H), 4.23 (d, J=2.4 Hz, 1H), 4.22-4.20 (m, 1H), 4.17 (d, J=7.2 Hz, 1H), 4.14 (d, J=6.4 Hz, 1H), 4.12-4.05 (m, 2H), 4.02-3.97 (m, 1H), 3.81 (d, J=4.0 Hz, 4H), 3.80-3.76 (m, 1H), 3.75-3.65 (m, 4H), 3.60-3.48 (m, 3H), 3.45-3.34 (m, 4H), 3.26-3.09 (m, 6H), 3.05-2.95 (m, 2H), 2.92-2.70 (m, 3H), 2.33-2.12 (m, 3H), 2.08-2.03 (m, 1H), 1.93-1.85 (m, 2H), 1.75-1.55 (m, 3H), 1.51-1.41 (m, 6H). ¹⁹F NMR (377 MHz, METHANOL-d₄) δ=−120.762.

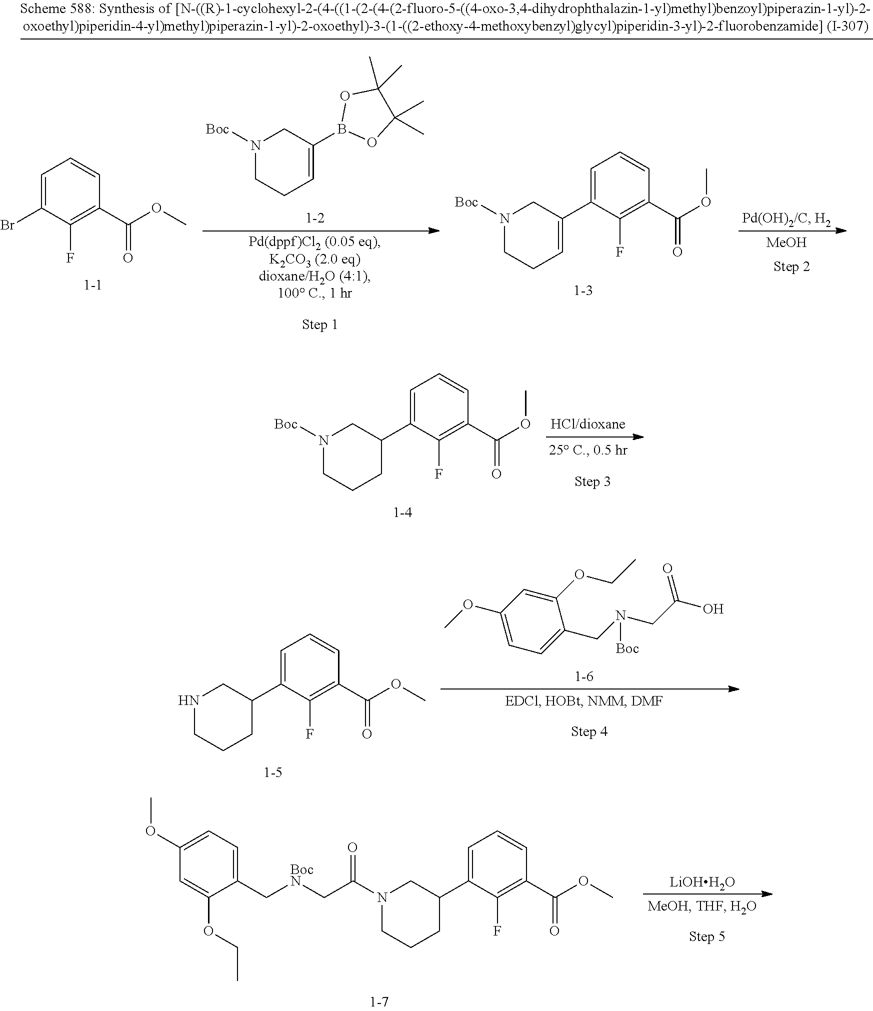

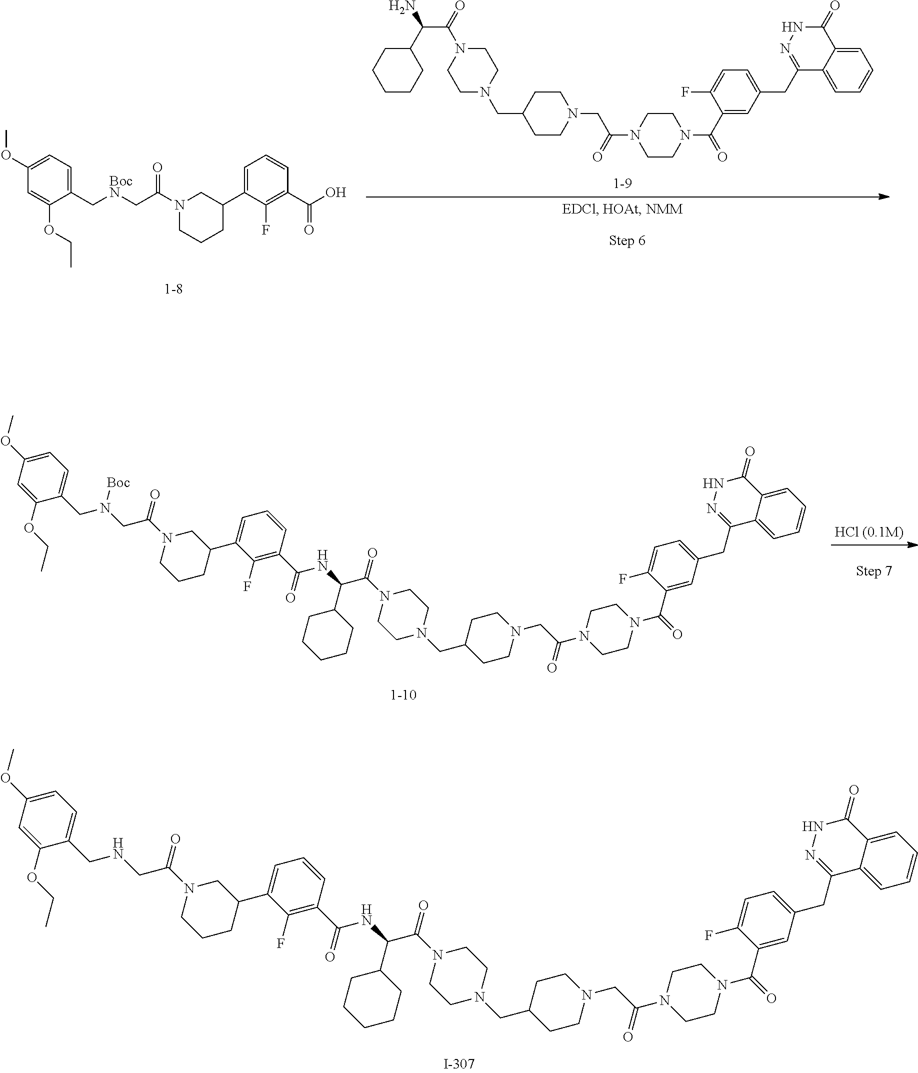 [SMILES: CCOc1cc(OC)ccc1CN(CC(=O)N1CCCC(c2cccc(C(=O)O)c2F)C1)C(=O)OC(C)(C)C ; N[C@@H](C(=O)N1CCN(CC2CCN(CC(=O)N3CCN(C(=O)c4cc(Cc5n[nH]c(=O)c6ccccc56)ccc4F)CC3)CC2)CC1)C1CCCCC1 ; CCOc1cc(OC)ccc1CN(CC(=O)N1CCCC(c2cccc(C(=O)N[C@@H](C(=O)N3CCN(CC4CCN(CC(=O)N5CCN(C(=O)c6cc(Cc7n[nH]c(=O)c8ccccc78)ccc6F)CC5)CC4)CC3)C3CCCCC3)c2F)C1)C(=O)OC(C)(C)C ; CCOc1cc(OC)ccc1CNCC(=O)N1CCCC(c2cccc(C(=O)N[C@@H](C(=O)N3CCN(CC4CCN(CC(=O)N5CCN(C(=O)c6cc(Cc7n[nH]c(=O)c8ccccc78)ccc6F)CC5)CC4)CC3)C3CCCCC3)c2F)C1]

### Step 1: Synthesis of Intermediate 1-3

A mixture of Intermediate 1-1 (1 g, 4.29 mmol, 1 eq), Intermediate 1-2 (1.33 g, 4.29 mmol, 1 eq), Pd(dppf)Cl₂ (157.00 mg, 214.56 μmol, 0.05 eq), and K₂CO₃ (1.19 g, 8.58 mmol, 2 eq) in dioxane (10 mL) and H₂O (2.5 mL) was degassed and purged with N₂ 3 times, and then the mixture was stirred at 100° C. for 1 hr under N₂ atmosphere. The reaction mixture was diluted with H₂O (20 mL) and extracted with EA (25 mL*2). The combined organic layers were washed with NaCl (aq) (15 mL*2), dried over Na₂SO₄, filtered and concentrated under reduced pressure to give a residue. The residue was purified by flash silica gel chromatography (ISCO; 40 g SepaFlash Silica Flash Column, Eluent of 0˜50% Ethyl acetate/Petroleum ether gradient @100 mL/min) and was concentrated under reduced pressure to give Intermediate 1-3 (1.4 g, 4.01 mmol, 93.53% yield, 96.146% purity) as a yellow oil. LCMS (Method D): Retention time: 0.478 min, [M+Na]+=358.1. ¹H NMR (400 MHz, CHLOROFORM-d) δ=7.83-7.71 (m, 1H), 7.41-7.31 (m, 1H), 7.11-7.07 (m, 1H), 5.95 (s, 1H), 4.11 (s, 2H), 3.86 (s, 3H), 3.52-3.49 (m, 2H), 2.25 (d, J=2.4 Hz, 2H), 1.41 (s, 9H).

### Step 2: Synthesis of Intermediate 1-4

To a solution of Intermediate 1-3 (1.3 g, 3.88 mmol, 1 eq) in MeOH (13 mL) was added Pd(OH)₂/C (600 mg, 20% purity) under N₂ atmosphere. The suspension was degassed under vacuum and purged with H₂ 3 times. The mixture was stirred under H₂ (15 psi) at 60° C. for 2 hr. The reaction mixture was filtered and concentrated under reduced pressure to give a residue. The residue was purified by flash silica gel chromatography (ISCO; 80 g SepaFlash Silica Flash Column, Eluent of 0˜20% ethyl acetate/Petroleum ether gradient @80 mL/min) and was concentrated under reduced pressure to give Intermediate 1-4 (1.3 g, 3.78 mmol, 97.41% yield, 98% purity) as a colorless oil. LCMS (Method D): Retention time: 0.472 min, [M+Na]+=360.0. ¹H NMR (400 MHz, CHLOROFORM-d) δ=7.82-7.76 (m, 1H), 7.45-7.37 (m, 1H), 7.18-7.14 (m, 1H), 5.31 (s, 1H), 4.14 (d, J=17.2 Hz, 2H), 3.94 (s, 3H), 3.12-3.00 (m, 1H), 2.90-2.82 (m, 1H), 2.05-1.95 (m, 1H), 1.80-1.63 (m, 3H), 1.61 (s, 2H), 1.47 (s, 9H).

### Step 3: Synthesis of Intermediate 1-5

A solution of Intermediate 1-4 (0.3 g, 889.19 μmol, 1 eq) in HCl/dioxane (3 mL) was stirred at 25° C. for 0.5 hr. The reaction mixture was concentrated under reduced pressure to give Intermediate 1-5 (0.3 g, crude, HCl salt) as a white solid. LCMS (Method D): Retention time: 0.188 min, [M+H]+=238.1.

### Step 4: Synthesis of Intermediate 1-7

To a solution of Intermediate 1-5 (100 mg, 365.32 μmol, 1 eq, HCl salt) and Intermediate 1-6 (148.78 mg, 438.39 μmol, 1.2 eq) in DMF (1 mL) was added EDCI (210.10 mg, 1.10 mmol, 3 eq), NMM (184.76 mg, 1.83 mmol, 200.82 μL, 5 eq), HOAt (49.72 mg, 365.32 μmol, 51.10 μL, 1 eq). The mixture was stirred at 25° C. for 1 hr. The reaction mixture was diluted with H₂O (5 mL) and extracted with EA (5 mL*2). The combined organic layers were washed with NaCl(aq) (5 mL*2), dried over Na₂SO₄, filtered and concentrated under reduced pressure to give a residue. The residue was purified by flash silica gel chromatography (ISCO; 12 g SepaFlash Silica Flash Column, Eluent of 0˜60% Ethyl acetate/Petroleum ether gradient @30 mL/min) and was concentrated under reduced pressure to give Intermediate 1-7 (100 mg, 169.25 μmol, 46.33% yield, 94.552% purity) as a colorless oil. LCMS (Method D): Retention time: 0.509 min, [M+H]+=559.4.

### Step 5: Synthesis of Intermediate 1-8

To a solution of Intermediate 1-7 (100 mg, 179.01 μmol, 1 eq) in THF (1 mL) and H₂O (1 mL) and MeOH (1 mL) was added LiOH·H₂O (22.54 mg, 537.02 μmol, 3 eq). The mixture was stirred at 25° C. for 1 hr. The reaction mixture was adjusted pH=6 by HCl (2M) and was concentrated to remove THF and lyophilized to give Intermediate 1-8 (80 mg, 146.89 μmol, 82.06% yield) as a white solid. LCMS (Method D): Retention time: 0.384 min, [M+H]+=545.3.

### Step 6: Synthesis of Intermediate 1-10

To a solution of Intermediate 1-9 (40 mg, 52.26 μmol, 1 eq, HCl salt) and Intermediate 1-8 (42.69 mg, 78.39 μmol, 1.5 eq) in DMF (1 mL) was added NMM (26.43 mg, 261.32 μmol, 28.73 μL, 5 eq), HOAt (7.11 mg, 52.26 μmol, 7.31 μL, 1 eq), EDCI (30.06 mg, 156.79 μmol, 3 eq). The mixture was stirred at 25° C. for 0.5 hr. The reaction mixture was diluted with H₂O (5 mL) and extracted with EA (5 mL*2). The combined organic layers were washed with NaCl (aq) (5 mL*2), dried over Na₂SO₄, filtered and concentrated under reduced pressure to give a residue. The residue was purified by prep-HPLC (0.1% FA) the eluent was concentrated to remove ACN and lyophilized to give Intermediate 1-10 (30 mg, 23.90 mol, 45.72% yield) as a white solid. LCMS (Method D): Retention time: 0.765 min, [M+H]+=1256.4. SFC: Retention time: 0.689 min, 0.902 min.

### Step 7: Synthesis of I-307

A solution of Intermediate 1-10 (20 mg, 15.93 μmol, 1 eq) in HCl (0.1 M, 0.2 mL, 1.26 eq) was stirred at 100° C. for 0.5 hr. The reaction mixture was concentrated under reduced pressure to give a residue. The residue was purified by prep-HPLC (0.1% FA) and the eluent was concentrated to remove ACN and lyophilized to give I-307 (10.41 mg, 8.80 μmol, 55.24% yield, 97.659% purity) as a white solid. LCMS (Method D): Retention time: 0.352 min, [M+H]+=1155.7. SFC: Retention time: 0.846 min, 1.146 min. ¹H NMR (400 MHz, METHANOL-d₄) δ=8.62-8.43 (m, 1H), 8.37 (d, J=7.2 Hz, 1H), 8.00-7.93 (m, 1H), 7.91-7.81 (m, 2H), 7.64-7.46 (m, 3H), 7.42-7.33 (m, 1H), 7.31-7.21 (m, 2H), 7.19-7.15 (m, 1H), 6.64-6.48 (m, 2H), 5.00-4.91 (m, 2H), 4.58 (s, 3H), 4.55 (s, 1H), 4.39 (s, 2H), 4.20-4.02 (m, 5H), 3.91-3.86 (m, 1H), 3.80 (d, J=3.6 Hz, 5H), 3.75-3.73 (m, 2H), 3.71-3.69 (m, 3H), 3.58-3.50 (m, 3H), 3.40-3.34 (m, 2H), 3.16-3.13 (m, 1H), 3.03-2.96 (m, 2H), 2.54-2.48 (m, 2H), 2.46-2.36 (m, 2H), 2.28-2.17 (m, 4H), 2.04-2.00 (m, 1H), 1.93-1.75 (m, 9H), 1.73-1.67 (m, 2H), 1.65-1.56 (m, 2H), 1.49-1.42 (m, 3H), 1.29 (d, J=4.4 Hz, 3H), 1.27-1.21 (m, 2H), 1.18-1.10 (m, 2H). ¹⁹F NMR (377 MHz, METHANOL-d₄) δ=−120.740, −121.663.

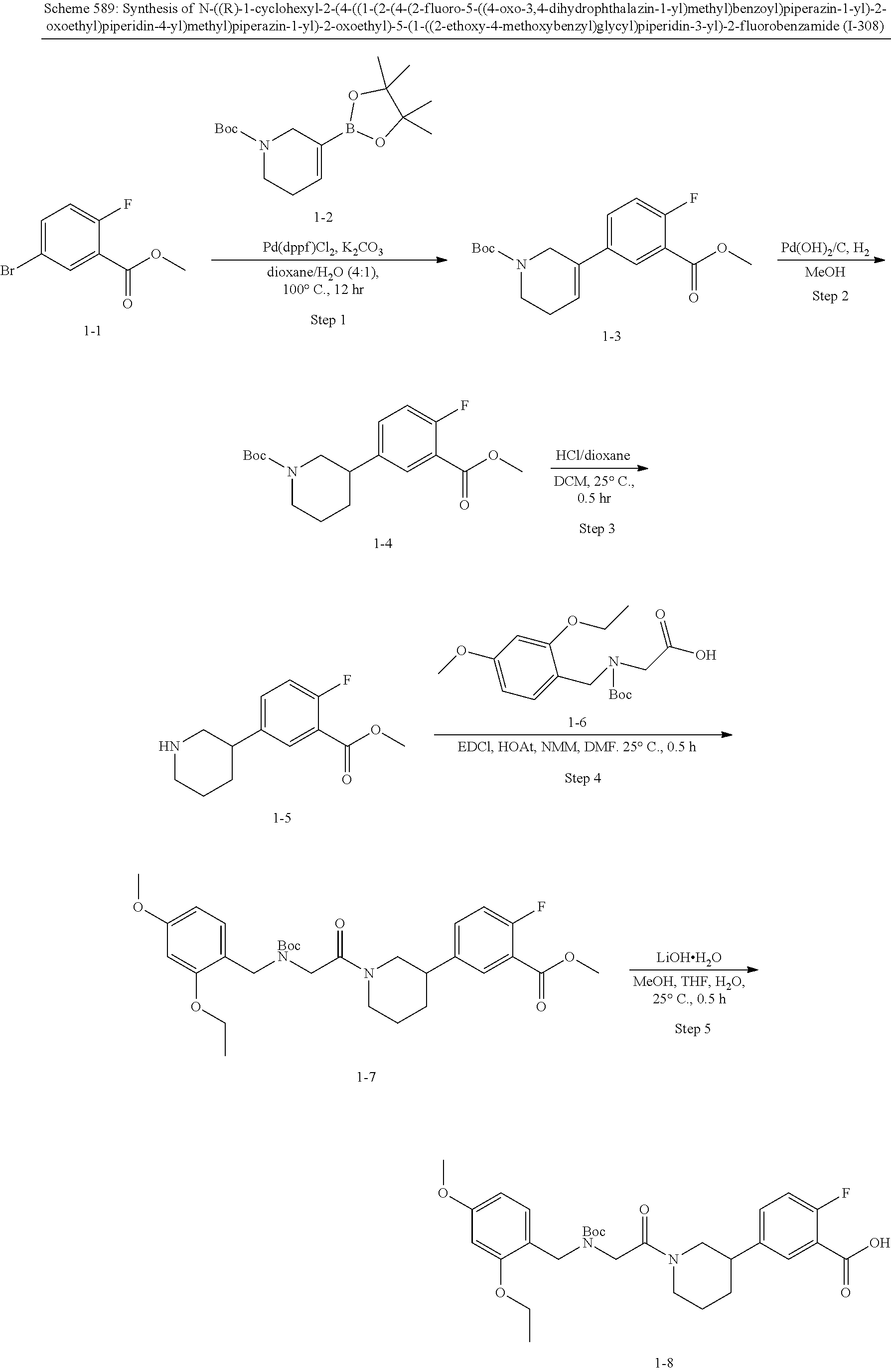

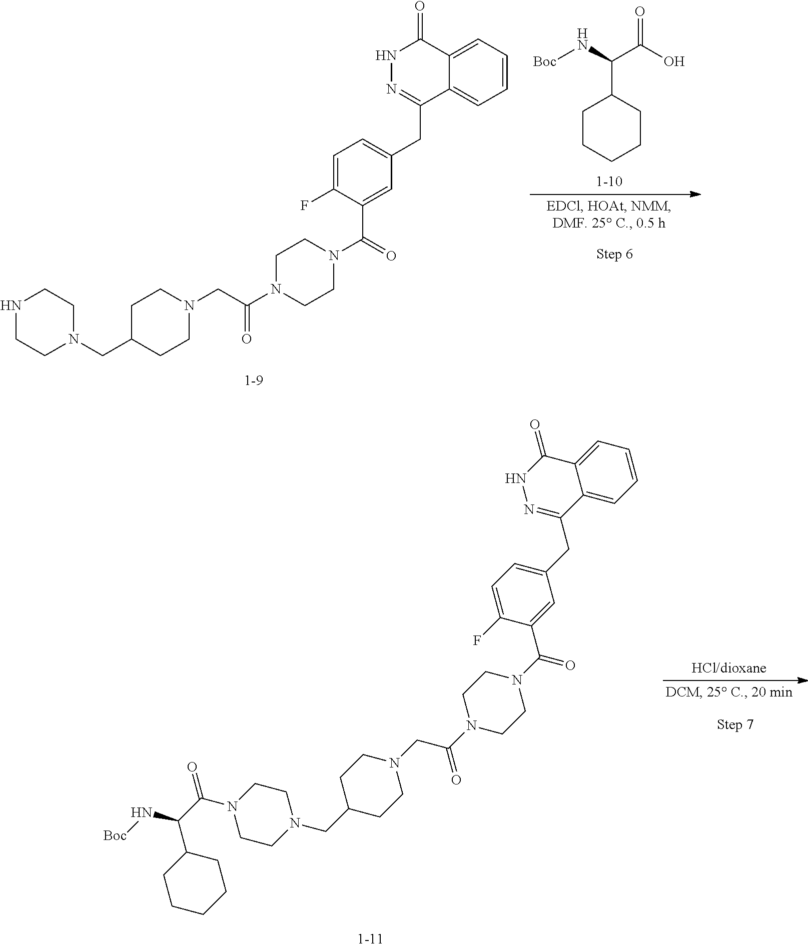 [SMILES: O=C(CN1CCC(CN2CCNCC2)CC1)N1CCN(C(=O)c2cc(Cc3n[nH]c(=O)c4ccccc34)ccc2F)CC1 ; CC(C)(C)OC(=O)N[C@@H](C(=O)O)C1CCCCC1 ; CC(C)(C)OC(=O)N[C@@H](C(=O)N1CCN(CC2CCN(CC(=O)N3CCN(C(=O)c4cc(Cc5n[nH]c(=O)c6ccccc56)ccc4F)CC3)CC2)CC1)C1CCCCC1]

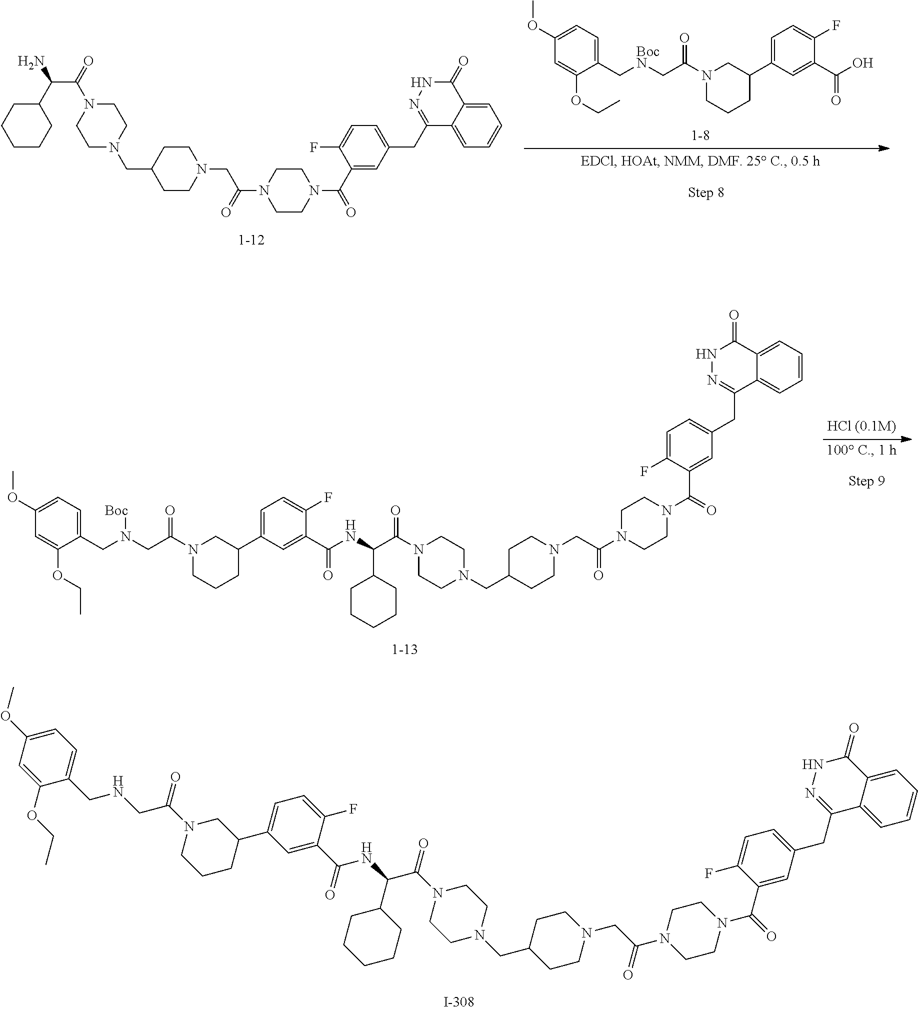 [SMILES: N[C@@H](C(=O)N1CCN(CC2CCN(CC(=O)N3CCN(C(=O)c4cc(Cc5n[nH]c(=O)c6ccccc56)ccc4F)CC3)CC2)CC1)C1CCCCC1 ; CCOc1cc(OC)ccc1CN(CC(=O)N1CCCC(c2ccc(F)c(C(=O)O)c2)C1)C(=O)OC(C)(C)C ; CCOc1cc(OC)ccc1CN(CC(=O)N1CCCC(c2ccc(F)c(C(=O)N[C@@H](C(=O)N3CCN(CC4CCN(CC(=O)N5CCN(C(=O)c6cc(Cc7n[nH]c(=O)c8ccccc78)ccc6F)CC5)CC4)CC3)C3CCCCC3)c2)C1)C(=O)OC(C)(C)C ; CCOc1cc(OC)ccc1CNCC(=O)N1CCCC(c2ccc(F)c(C(=O)N[C@@H](C(=O)N3CCN(CC4CCN(CC(=O)N5CCN(C(=O)c6cc(Cc7n[nH]c(=O)c8ccccc78)ccc6F)CC5)CC4)CC3)C3CCCCC3)c2)C1]

### Step 1: Synthesis of Intermediate 1-3

To a solution of Intermediate 1-1 (600 mg, 2.57 mmol, 1 eq) and Intermediate 1-2 (955.35 mg, 3.09 mmol, 1.2 eq) in dioxane (5 mL) and H₂O (1 mL) was added Pd(dppf)Cl₂ (188.39 mg, 257.47 μmol, 0.1 eq) and K₂CO₃ (711.68 mg, 5.15 mmol, 2 eq). The mixture was stirred at 100° C. for 12 hr. The mixture was diluted with water (10 mL) and extracted with DCM (10 mL*3). The combined organic layer was dried over Na₂SO₄, filtered and concentrated in vacuum to give a residue. The residue was purified by column chromatography (SiO₂, PE:EA=20:1 to PE:EA=0:1), then the organic liquid was concentrated in vacuum to give a residue. Intermediate 1-3 (800 mg, 2.19 mmol, 85.24% yield, 92% purity) was obtained as colorless oil. LCMS (Method D): Retention time=0.495 min, [M+Na]+=358.2. ¹H NMR (400 MHz, METHANOL-d₄) δ=7.91 (d, J=5.8 Hz, 1H), 7.63 (d, J=3.2 Hz, 1H), 7.20 (t, J=9.6 Hz, 1H), 6.27 (s, 1H), 4.25 (s, 2H), 3.92 (s, 3H), 3.56 (t, J=5.6 Hz, 2H), 2.32 (d, J=2.0 Hz, 2H), 1.50 (s, 9H).

### Step 2: Synthesis of Intermediate 1-4

To a solution of Intermediate 1-3 (500 mg, 1.49 mmol, 1 eq) in MeOH (1 mL) was added Pd(OH)₂/C (190 mg, 1.49 mmol, 20% purity, 1 eq) under N₂. The suspension was degassed and purged with H₂ 3 times. The mixture was stirred under H₂ (15 Psi) at 60° C. for 1 hr. The reaction mixture was filtered and concentrated in vacuum to give a residue. The residue was used in the next step directly without further purification. Intermediate 1-4 (500 mg, 1.48 mmol, 99.40% yield) was obtained as a colorless oil. LCMS (Method D): Retention time=0.469 min, [M+Na]+=360.0.

### Step 3: Synthesis of Intermediate 1-5

To a solution of Intermediate 1-4 (120 mg, 355.68 μmol, 1 eq) in DCM (0.5 mL) was added HCl/dioxane (2 M, 1 mL, 5.62 eq). The mixture was stirred at 25° C. for 0.5 hr. The reaction mixture was concentrated in vacuum to give a residue. The residue was used in the next step directly without further purification. Intermediate 1-5 (90 mg, 328.79 μmol, 92.44% yield, HCl salt) was obtained as a white solid. LCMS (Method D): Retention time=0.267 min, [M+H]+=238.1.

### Step 4: Synthesis of Intermediate 1-7

To a solution of Intermediate 1-5 (90 mg, 328.79 μmol, 1 eq, HCl salt) and Intermediate 1-6 (133.90 mg, 394.55 μmol, 1.2 eq) in DMF (1 mL) was added EDCI (189.09 mg, 986.37 μmol, 3 eq), HOAt (44.75 mg, 328.79 μmol, 45.99 μL, 1 eq), NMM (166.28 mg, 1.64 mmol, 180.74 μL, 5 eq). The mixture was stirred at 25° C. for 0.5 hr. The mixture was diluted with water (5 mL) and extracted with DCM (5 mL*3). The combined organic layer was dried over Na₂SO₄, filtered and concentrated in vacuum to give a residue. The residue was purified by column chromatography (SiO₂, PE:EA=5:1 to EA:MeOH=0:1), then the organic liquid was concentrated in vacuum to give a residue. Intermediate 1-7 (160 mg, 274.96 mol, 83.63% yield, 96% purity) was obtained as a colorless oil. LCMS (Method D): Retention time=0.512 min, [M+H]+=559.1.

### Step 5: Synthesis of Intermediate 1-8

To a solution of methyl Intermediate 1-7 (160 mg, 286.41 μmol, 1 eq) in MeOH (0.4 mL), THF (0.4 mL), and H₂O (0.4 mL) was added LiOH·H₂O (24.04 mg, 572.82 μmol, 2 eq). The mixture was stirred at 25° C. for 0.5 hr. The reaction mixture was concentrated in vacuum to remove THF and MeOH to give a residue, the residue was diluted with water (5 mL) and extracted with DCM (5 mL*3), then the aqueous phase was acidified with hydrochloric acid (1 M) to pH=5-6, the residue was extracted with DCM (5 mL*3). The combined organic layer was dried over Na₂SO₄, filtered and concentrated in vacuum to give a residue. The residue was used in the next step directly without further purification. Intermediate 1-8 (150 mg, 275.43 μmol, 96.16% yield) was obtained as a colorless gum. LCMS (Method D): Retention time=0.470 min, [M+H]+=545.4.

### Step 6: Synthesis of Intermediate 1-11

To a solution of Intermediate 1-9 (150 mg, 254.37 μmol, 1 eq) and Intermediate 1-10 (78.55 mg, 305.24 μmol, 1.2 eq) in DMF (2 mL) was added EDCI (146.29 mg, 763.10 μmol, 3 eq), HOAt (34.62 mg, 254.37 μmol, 35.58 μL, 1 eq), NMM (128.64 mg, 1.27 mmol, 139.83 μL, 5 eq). The mixture was stirred at 25° C. for 0.5 hr. The mixture was diluted with water (5 mL) and extracted with DCM (5 mL*3). The combined organic layer was dried over Na₂SO₄, filtered and concentrated in vacuum to give a residue. The residue was purified by reverse phase chromatography (0.1% FA condition), then the residue was concentrated in vacuum and lyophilized. Intermediate 1-11 (100 mg, 118.21 μmol, 46.47% yield, 98% purity) was obtained as a white solid. LCMS (Method D): Retention time=0.321 min, [M+H]+=829.7. SFC: Retention time=1.405 min.

### Step 7: Synthesis of Intermediate 1-12

To a solution of Intermediate 1-11 (90 mg, 108.56 μmol, 1 eq) in DCM (0.5 mL) was added HCl/dioxane (2 M, 0.5 mL, 9.21 eq). The mixture was stirred at 25° C. for 20 min. The reaction mixture was concentrated in vacuum to give a residue. The residue was used in the next step directly without further purification. Intermediate 1-12 (100 mg, crude, HCl salt) was obtained as a yellow solid. LCMS (Method D): Retention time=0.250 min, [M+H]+=729.5. SFC: Retention time=0.914 min.

### Step 8: Synthesis of Intermediate 1-13

To a solution of Intermediate 1-12 (50 mg, 65.33 μmol, 1 eq, HCl salt) and Intermediate 1-8 (42.69 mg, 78.39 μmol, 1.2 eq) in DMF (0.5 mL) was added EDCI (37.57 mg, 195.99 μmol, 3 eq), HOAt (8.89 mg, 65.33 μmol, 9.14 μL, 1 eq), NMM (33.04 mg, 326.64 μmol, 35.91 μL, 5 eq). The mixture was stirred at 25° C. for 0.5 hr. The mixture was diluted with water (5 mL) and extracted with DCM (5 mL*3). The combined organic layer was dried over Na₂SO₄, filtered and concentrated in vacuum to give a residue. The residue was purified by prep-HPLC (column: Phenomenex luna C18 150*25 mm*10 um; mobile phase: [water (FA)-ACN]; gradient: 24%-54% B over 20 min). The eluent was concentrated in vacuum and lyophilized. Intermediate 1-13 (40 mg, 31.86 μmol, 48.77% yield) was obtained as a white solid. LCMS (Method D): Retention time=0.413 min, [M/2+H]+=628.6. SFC: Retention time=0.993 min.

### Step 9: Synthesis of I-308

A solution of Intermediate 1-13 (30 mg, 23.90 μmol, 1 eq) in HCl (0.1 M, 0.5 mL, 2.09 eq) was stirred at 100° C. for 1 hr. The mixture was diluted with water (5 mL) and extracted with DCM (5 mL*3). The combined organic layer was dried over Na₂SO₄, filtered and concentrated in vacuum to give a residue. The residue was purified by reverse phase (0.1% TFA condition), then the residue was concentrated in vacuum and lyophilized. I-308 (25.83 mg, 19.47 μmol, 81.47% yield, 95.668% purity, TFA salt) was obtained as a white solid. LCMS (Method D): Retention time=0.378 min, [M+H]+=1155.6. SFC: Retention time=1.057 min. ¹H NMR (400 MHz, METHANOL-d₄) δ=8.37 (d, J=8.4 Hz, 1H), 7.97-7.92 (m, 1H), 7.90-7.81 (m, 2H), 7.66-7.59 (m, 1H), 7.51-7.48 (m, J=6.7, 2H), 7.39-7.33 (m, 1H), 7.32-7.26 (m, 1H), 7.24-7.15 (m, 2H), 6.63 (s, 1H), 6.58-6.57 (m, 1H), 4.88 (s, 2H), 4.58-4.50 (m, 1H), 4.39 (s, 2H), 4.30 (s, 1H), 4.21 (d, J=9.6 Hz, 3H), 4.19-4.14 (m, 2H), 4.12 (d, J=11.6 Hz, 1H), 4.05 (d, J=8.4 Hz, 2H), 3.99-3.94 (m, 1H), 3.82 (s, 4H), 3.79-3.76 (m, 1H), 3.71-3.68 (m, 4H), 3.58-3.48 (m, 3H), 3.39-3.34 (m, 4H), 3.22-3.15 (m, 2H), 3.14-3.03 (m, 4H), 2.83-2.70 (m, 2H), 2.30-2.20 (m, 1H), 2.19-1.97 (m, 4H), 1.87 (s, 3H), 1.81-1.79 (m, 4H), 1.75-1.66 (m, 4H), 1.50-1.42 (m, 3H), 1.33-1.26 (m, 3H), 1.24-1.10 (m, 3H). ¹⁹F NMR (377 MHz, METHANOL-d₄) δ=−77.097, −120.682, −120.764.

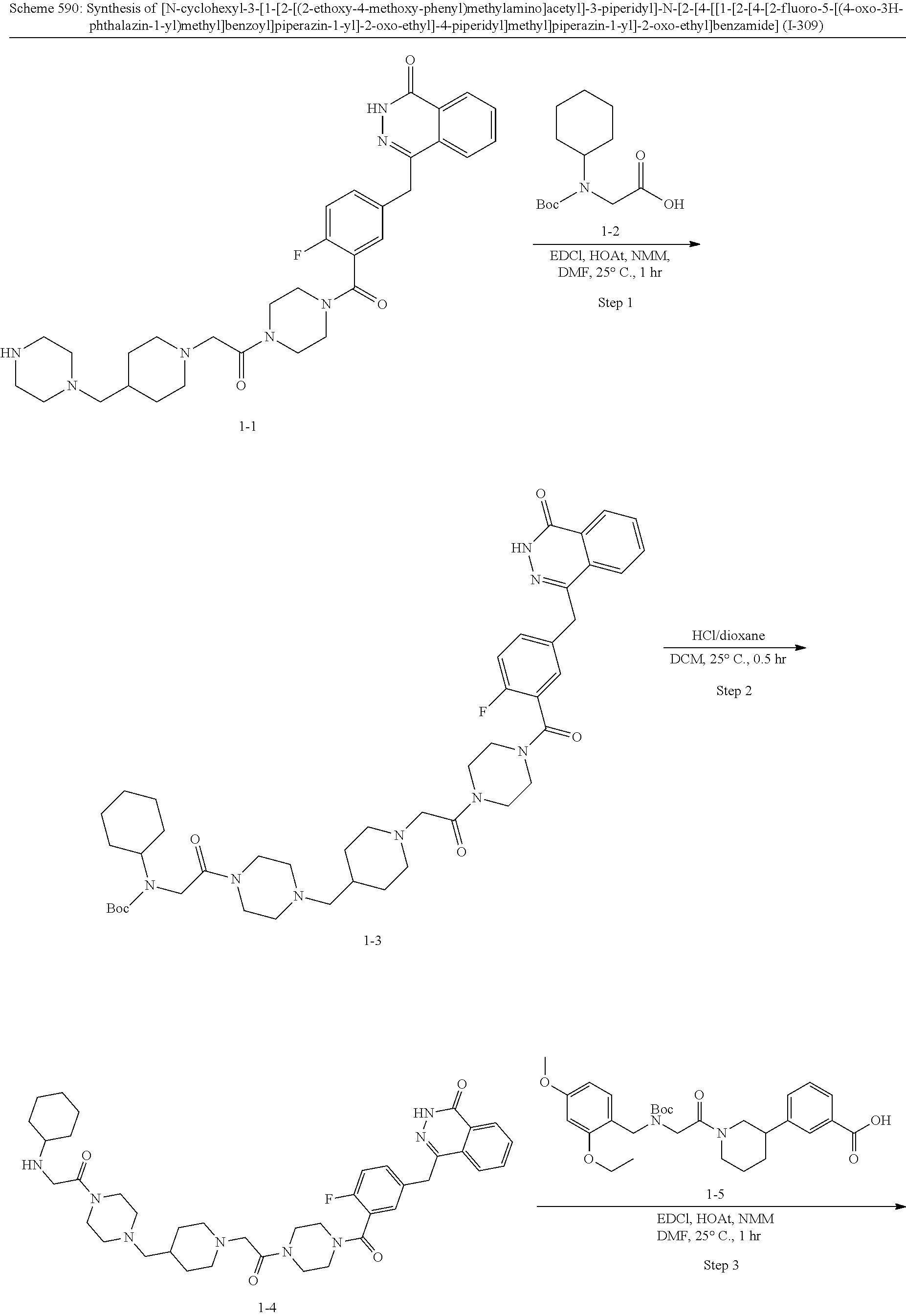 [SMILES: O=C(CN1CCC(CN2CCNCC2)CC1)N1CCN(C(=O)c2cc(Cc3n[nH]c(=O)c4ccccc34)ccc2F)CC1 ; CC(C)(C)OC(=O)N(CC(=O)O)C1CCCCC1 ; CC(C)(C)OC(=O)N(CC(=O)N1CCN(CC2CCN(CC(=O)N3CCN(C(=O)c4cc(Cc5n[nH]c(=O)c6ccccc56)ccc4F)CC3)CC2)CC1)C1CCCCC1 ; O=C(CNC1CCCCC1)N1CCN(CC2CCN(CC(=O)N3CCN(C(=O)c4cc(Cc5n[nH]c(=O)c6ccccc56)ccc4F)CC3)CC2)CC1 ; CCOc1cc(OC)ccc1CN(CC(=O)N1CCCC(c2cccc(C(=O)O)c2)C1)C(=O)OC(C)(C)C]

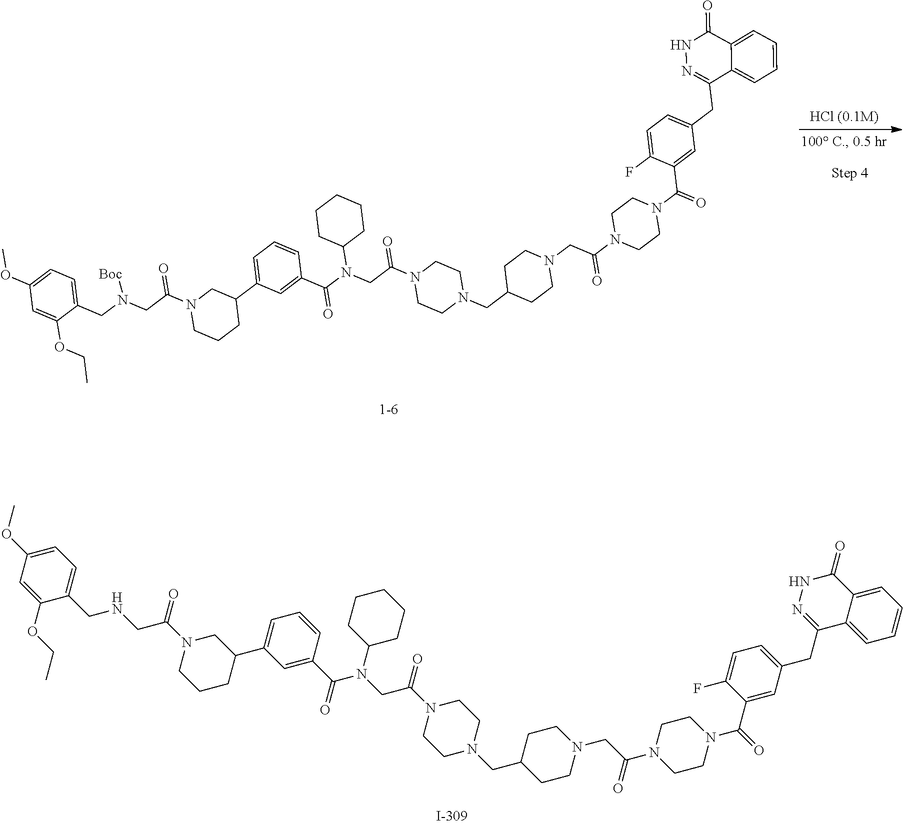 [SMILES: CCOc1cc(OC)ccc1CN(CC(=O)N1CCCC(c2cccc(C(=O)N(CC(=O)N3CCN(CC4CCN(CC(=O)N5CCN(C(=O)c6cc(Cc7n[nH]c(=O)c8ccccc78)ccc6F)CC5)CC4)CC3)C3CCCCC3)c2)C1)C(=O)OC(C)(C)C ; CCOc1cc(OC)ccc1CNCC(=O)N1CCCC(c2cccc(C(=O)N(CC(=O)N3CCN(CC4CCN(CC(=O)N5CCN(C(=O)c6cc(Cc7n[nH]c(=O)c8ccccc78)ccc6F)CC5)CC4)CC3)C3CCCCC3)c2)C1]

### Step 1: Synthesis of Intermediate 1-3.

To a mixture of Intermediate 1-1 (150 mg, 254.37 μmol, 1 eq) in DMF (1.5 mL) was added Intermediate 1-2 (78.55 mg, 305.24 μmol, 1.2 eq), EDCI (146.29 mg, 763.10 μmol, 3 eq), HOAt (34.62 mg, 254.37 μmol, 35.58 μL, 1 eq) and NMM (128.64 mg, 1.27 mmol, 139.83 μL, 5 eq) at 25° C., and the mixture was stirred at 25° C. for 1 h. The reaction mixture was diluted with H₂O (3 mL), extracted with EtOAc (2 mL×3), the organic layers were dried over Na₂SO₄, filtered and concentrated to give a residue. The residue was purified by column chromatography (SiO₂, EA/MeOH=1/0 to 1/1) and concentrated to give the product. Intermediate 1-3 (130 mg, 156.81 μmol, 61.65% yield) was obtained as a yellow solid. LCMS (Method D): Retention time: 0.306 min, [M+H]+=829.4.

### Step 2: Synthesis of Intermediate 1-4.

To a mixture of Intermediate 1-3 (110 mg, 132.69 μmol, 1 eq) in DCM (0.5 mL) was added HCl/dioxane (2 M, 0.5 mL, 7.54 eq) at 25° C. and the mixture was stirred at 25° C. for 0.5 h. The reaction mixture was concentrated to give a crude product. The crude product was used for the next step directly. Intermediate 1-4 (100 mg, 130.66 μmol, 98.47% yield, HCl salt) was obtained as a white solid. LCMS (Method D): Retention time: 0.248 min, [M+H]+=729.6.

### Step 3: Synthesis of Intermediate 1-6.

To a mixture of Intermediate 1-4 (100 mg, 130.66 μmol, 1 eq, HCl salt) in DMF (1 mL) was added Intermediate 1-5 (103.21 mg, 195.99 μmol, 1.5 eq), EDCI (75.14 mg, 391.97 μmol, 3 eq), HOAt (17.78 mg, 130.66 μmol, 18.28 μL, 1 eq) and NMM (66.08 mg, 653.29 μmol, 71.82 μL, 5 eq) at 25° C., and the mixture was stirred at 25° C. for 1 h. The reaction mixture was concentrated to give a residue. The residue was purified by reversed-phase HPLC (0.1% FA condition) and lyophilized to give the product. Intermediate 1-6 (37 mg, 29.90 μmol, 22.88% yield) was obtained as a yellow solid. LCMS (Method D): Retention time: 0.394 min, [M+H]+=1237.6.

### Step 4: Synthesis of I-309.

A mixture of Intermediate 1-6 (32 mg, 25.86 μmol, 1 eq) in HCl (0.1 M, 0.5 mL, 1.93 eq) was stirred at 100° C. for 0.5 h. The reaction mixture was concentrated to give a residue. The residue was purified by reversed-phase HPLC (0.1% FA condition) and lyophilized to give the product. I-309 (20 mg, 16.90 μmol, 65.36% yield, FA salt) was obtained as a white solid. LCMS (Method D): Retention time: 0.352 min, [M+H]+=1137.7. SFC: Retention time: 4.429 min, 5.105 min. ¹H NMR (400 MHz, METHANOL-d4) δ=8.48 (s, 1H), 8.37 (d, J=7.2 Hz, 1H), 7.99-7.92 (m, 1H), 7.91-7.77 (m, 2H), 7.50 (s, 1H), 7.47-7.25 (m, 6H), 7.17 (t, J=8.8 Hz, 1H), 6.62 (s, 1H), 6.56 (d, J=6.4 Hz, 1H), 4.53 (d, J=12.8 Hz, 1H), 4.39 (s, 2H), 4.29 (s, 1H), 4.28-4.07 (m, 5H), 4.07-3.91 (m, 2H), 3.85-3.59 (m, 13H), 3.58-3.35 (m, 5H), 3.29-3.04 (m, 4H), 2.93-2.70 (m, 2H), 2.61-2.38 (m, 5H), 2.29 (s, 2H), 2.19-1.99 (m, 2H), 1.94-1.70 (m, 9H), 1.67-1.29 (m, 10H), 1.10-0.98 (m, 2H). ¹⁹F NMR (377 MHz, METHANOL-d4) δ=−120.794

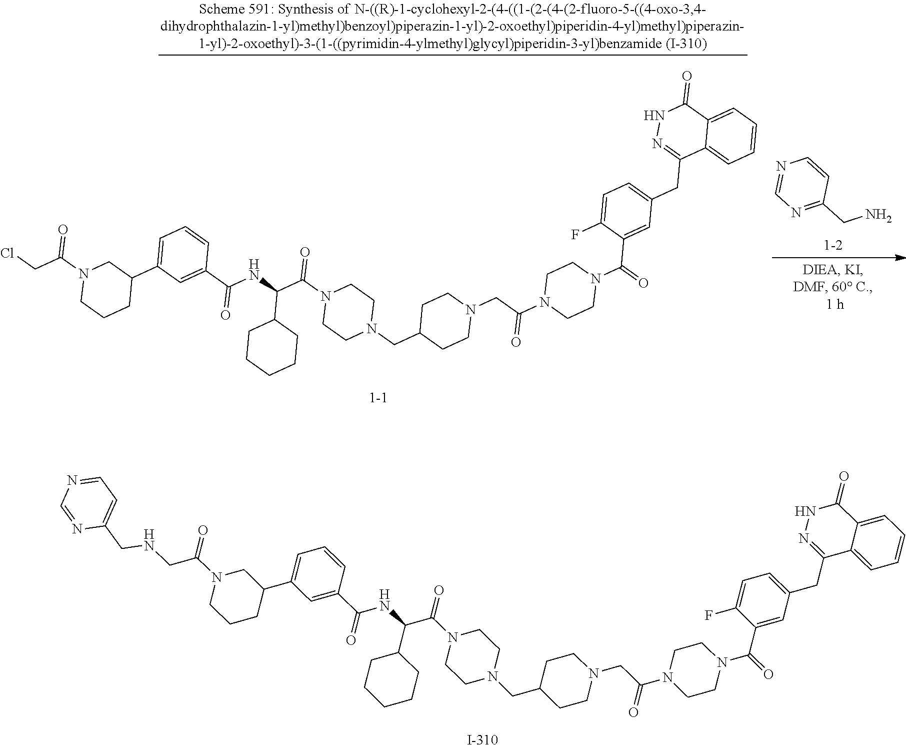

To a solution of Intermediate 1-1 (50 mg, 50.37 μmol, 1 eq) and Intermediate 1-2 (8.25 mg, 75.56 μmol, 1.5 eq) in DMF (1 mL) was added KI (33.45 mg, 201.49 μmol, 4 eq) and DIEA (26.04 mg, 201.49 μmol, 35.09 μL, 4 eq). The mixture was stirred at 60° C. for 1 hr. The reaction was filtered to give a residue. The residue was purified by prep-HPLC (column: YMC-Actus Triart C18 150*30 mm*7 um; mobile phase: [water (FA)-ACN]; gradient: 10%-40% B over 10 min). The eluent was concentrated under reduced pressure to remove ACN, then lyophilized to afford product. The product was further purified by prep-HPLC (column: Waters Xbridge 150*25 mm*5 um; mobile phase: [water (NH₄HCO₃)-ACN]; gradient: 32%-62% B over 9 min). The eluent was concentrated under reduced pressure to remove ACN, then lyophilization afforded I-310 (15 mg, 14.08 μmol, 27.95% yield, 100% purity) as a white solid. LCMS (Method G): Rt=0.589 min, M+H=1065.7. SFC: Rt=6.726 min, 8.211 min. ¹H NMR (400 MHz, METHANOL-d₄) δ=9.08 (d, J=11.2 Hz, 1H), 8.77-8.64 (m, 1H), 8.37 (d, J=7.6 Hz, 1H), 8.01-7.93 (m, 1H), 7.91-7.80 (m, 2H), 7.80-7.74 (m, 1H), 7.73-7.69 (m, 1H), 7.62-7.55 (m, 1H), 7.49 (d, J=6.4 Hz, 2H), 7.46-7.40 (m, 1H), 7.40-7.35 (m, 1H), 7.20-7.13 (m, 1H), 4.62-4.55 (m, 2H), 4.39 (s, 2H), 3.95 (d, J=10.4 Hz, 2H), 3.91-3.78 (m, 3H), 3.77-3.60 (m, 7H), 3.59-3.49 (m, 4H), 3.27-3.14 (m, 3H), 2.95-2.86 (m, 2H), 2.84-2.68 (m, 2H), 2.53-2.35 (m, 4H), 2.26-2.17 (m, 2H), 2.11-2.02 (m, 3H), 1.93-1.83 (m, 4H), 1.82-1.73 (m, 4H), 1.63 (d, J=1.6 Hz, 4H), 1.36-1.20 (m, 6H), 1.17-1.06 (m, 2H).

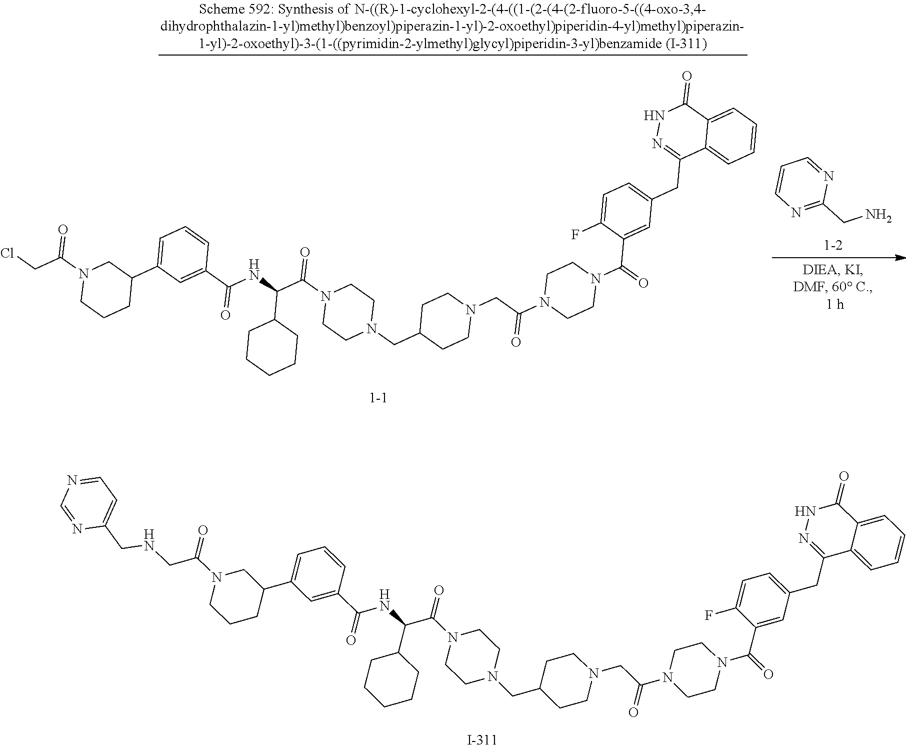

To a solution of Intermediate 1-1 (50 mg, 50.37 μmol, 1 eq) and Intermediate 1-2 (8.25 mg, 75.56 μmol, 1.5 eq) in DMF (1 mL) was added KI (33.45 mg, 201.49 μmol, 4 eq) and DIEA (26.04 mg, 201.49 μmol, 35.09 μL, 4 eq). The mixture was stirred at 60° C. for 1 hr. The reaction was filtered and concentrated under reduced pressure to give a residue. The residue was purified by prep-HPLC (column: YMC-Actus Triart C18 150*30 mm*7 um; mobile phase: [water (FA)-ACN]; gradient: 10%-40% B over 10 min). The eluent was concentrated under reduced pressure to remove ACN, then lyophilization afforded I-311 (27 mg, 25.35 μmol, 50.32% yield, 100% purity) as a white solid. LCMS (Method E): Rt=0.420 min, M+H=1065.6. SFC: Rt=4.226 min, 4.968 min. ¹H NMR (400 MHz, METHANOL-d₄) δ=8.88 (d, J=3.2 Hz, 1H), 8.87-8.79 (m, 1H), 8.44-8.31 (m, 1H), 8.00-7.93 (m, 1H), 7.93-7.75 (m, 3H), 7.73 (br d, J=7.3 Hz, 1H), 7.62-7.31 (m, 5H), 7.19-7.15 (m, 1H), 4.59-4.52 (m, 2H), 4.40 (s, 2H), 4.37-4.25 (m, 3H), 3.96-3.71 (m, 7H), 3.67-3.50 (m, 6H), 3.40 (br s, 2H), 3.20-3.08 (m, 2H), 2.99-2.77 (m, 2H), 2.76-2.37 (m, 7H), 2.15-1.84 (m, 9H), 1.78 (br d, J=8.0 Hz, 2H), 1.74-1.53 (m, 5H), 1.46 (d, J=2.4 Hz, 2H), 1.35-1.22 (m, 3H), 1.22-1.09 (m, 2H).

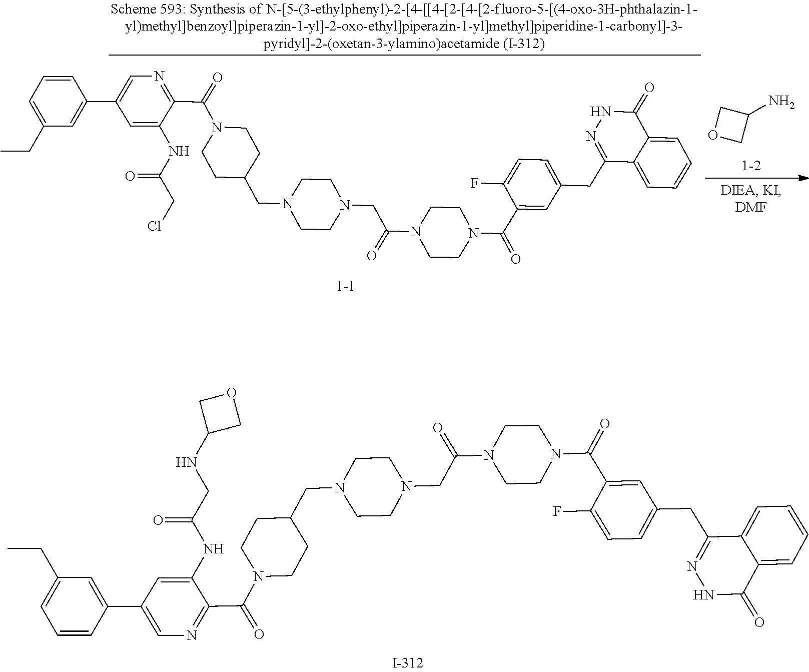

To the solution of Intermediate 1-1 (0.1 g, 112.30 μmol, 1 eq) in DMF (1 mL) was added Intermediate 1-2 (12.31 mg, 168.46 μmol, 1.5 eq) and DIEA (58.06 mg, 449.22 μmol, 78.24 μL, 4 eq) and KI (74.57 mg, 449.22 μmol, 4 eq), and the mixture was stirred at 25° C. for 12 hr. To the mixture was added MeOH (1 mL). The residue was purified by Prep-HPLC (column: Waters Xbridge 150*25 mm*5 um; mobile phase: [water (NH₄HCO₃)-ACN]; gradient: 22%-52% B over 15 min) and it was dried by lyophilization. I-312 (5.7 mg, 5.97 μmol, 5.31% yield, 97.075% purity) was obtained as a white solid. LCMS (Method G): Rt=0.581 min, (M+H)=927.5. ¹H NMR (400 MHz, CHLOROFORM-d) δ=10.65 (s, 1H), 10.50-10.41 (m, 1H), 8.98 (s, 1H), 8.53 (d, J=1.6 Hz, 1H), 8.50-8.43 (m, 1H), 7.82-7.68 (m, 3H), 7.46 (br s, 2H), 7.43-7.38 (m, 1H), 7.37-7.30 (m, 2H), 7.28 (s, 1H), 7.06-7.05 (m, 1H), 4.87-4.86 (m, 2H), 4.74 (br d, J=13.2 Hz, 1H), 4.55-4.53 (m, 2H), 4.29 (s, 2H), 4.15-3.98 (m, 2H), 3.87-3.67 (m, 4H), 3.59 (br d, J=18.8 Hz, 2H), 3.45 (s, 2H), 3.39-3.25 (m, 2H), 3.23-3.14 (m, 2H), 3.09-3.08 (m, 1H), 2.84-2.83 (m, 1H), 2.74-2.72 (m, 2H), 2.60-2.36 (m, 8H), 2.26-2.18 (m, 2H), 2.01 (s, 2H), 1.97-1.90 (m, 1H), 1.80 (br s, 1H), 1.32-1.23 (m, 5H).

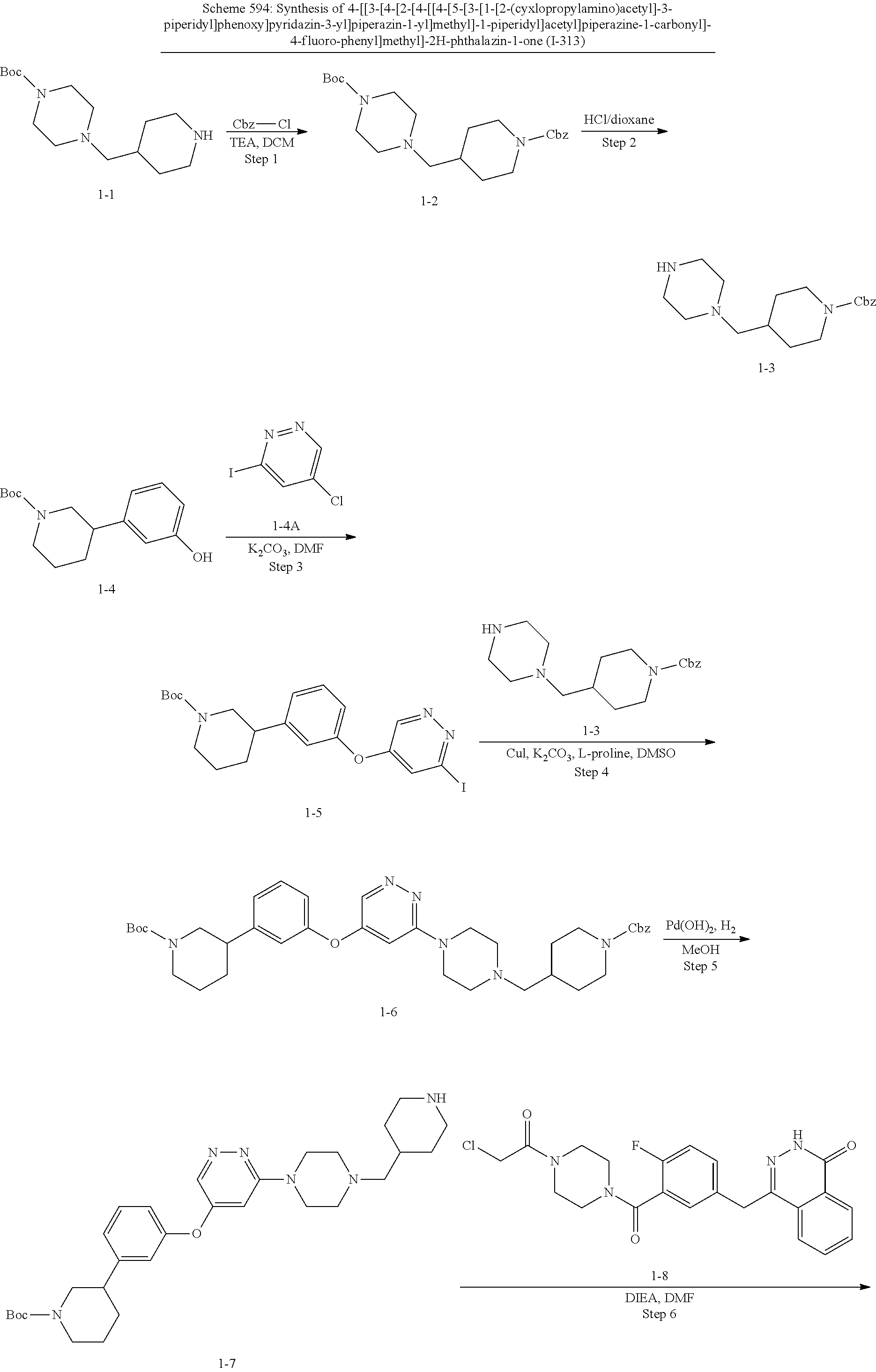

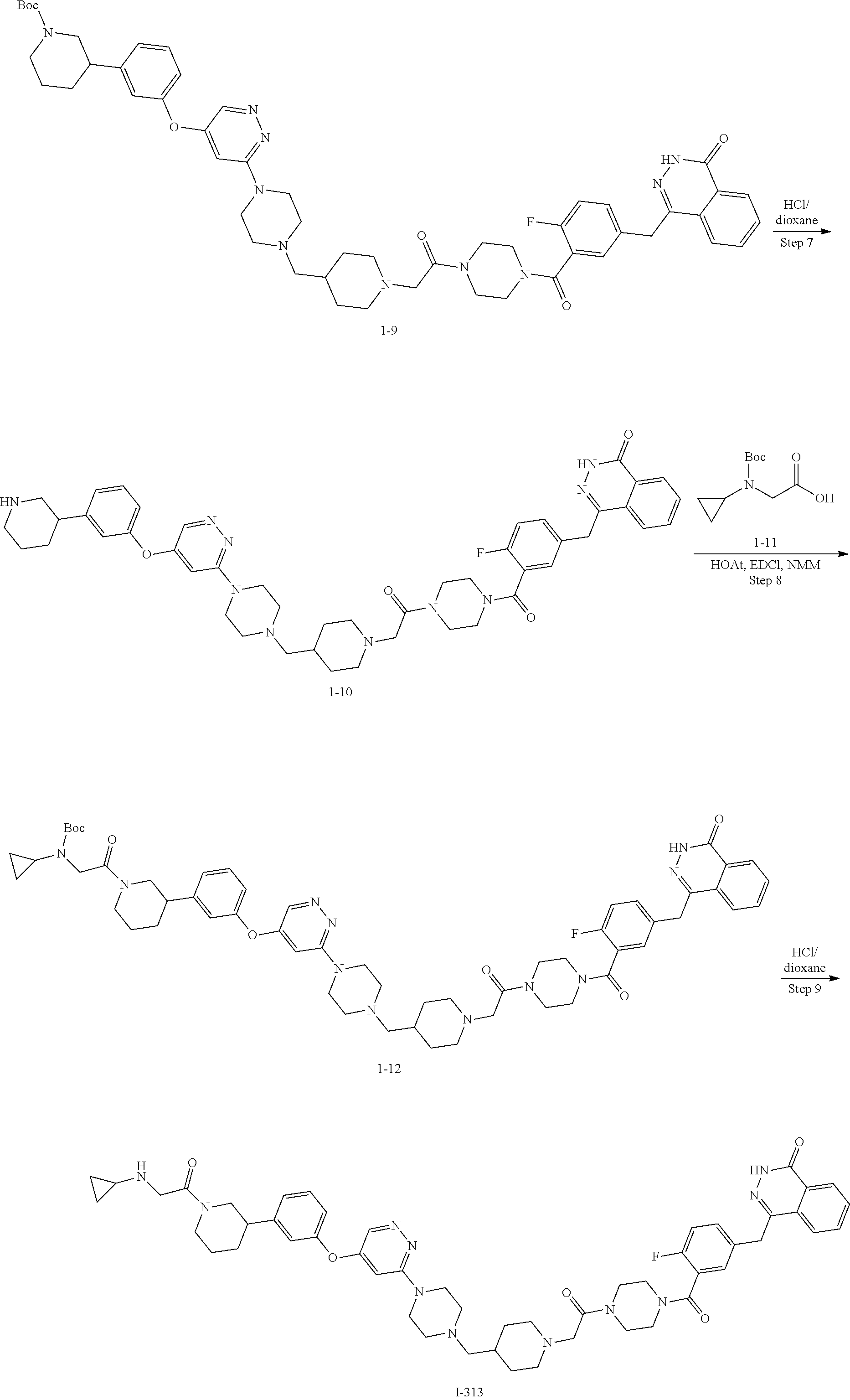

### Step 1: Synthesis of Intermediate 1-2.

To a solution of Intermediate 1-1 (2.5 g, 8.82 mmol, 1 eq) in DCM (25 mL) was added CbzCl (2.26 g, 13.23 mmol, 1.89 mL, 1.5 eq) and TEA (1.34 g, 13.23 mmol, 1.84 mL, 1.5 eq) at 0° C. The mixture was stirred at 20° C. for 1 hour. The reaction mixture was diluted with water (100 mL) and extracted with DCM (30 mL*2). The combined organic layers were concentrated under reduced pressure to give a residue. The residue was purified by flash silica gel chromatography (ISCO; 40 g SepaFlash Silica Flash Column, Eluent of 0˜30% Ethyl acetate/Petroleum ether gradient @80 mL/min). The eluent was concentrated under reduced pressure to give Intermediate 1-2 (2 g, 4.69 mmol, 53.16% yield, 97.9% purity) as a white solid. LCMS (Method D): Retention time: 0.426 min, [M+H]+=418.4.

### Step 2: Synthesis of Intermediate 1-3.

To a mixture of Intermediate 1-2 (2 g, 4.79 mmol, 1 eq) in DCM (30 mL) was added HCl/dioxane (2 M, 11.97 mL, 5 eq), then the mixture was stirred at 20° C. for 1 hour. The reaction mixture was concentrated under reduced pressure to give Intermediate 1-3 (1.2 g, 3.78 mmol, 78.92% yield) as a white solid.

### Step 3: Synthesis of Intermediate 1-5.

To a solution of Intermediate 1-4 (1.15 g, 4.16 mmol, 1 eq) and Intermediate 1-4A (1 g, 4.16 mmol, 1 eq) in DMF (10 mL) was added K₂CO₃ (1.72 g, 12.48 mmol, 3 eq). The mixture was stirred at 70° C. for 2 hours. The reaction mixture was diluted with water (80 mL) and extracted with EA (20 mL*3). The combined organic layers were concentrated under reduced pressure to give a residue. The residue was purified by flash silica gel chromatography (ISCO; 40 g SepaFlash Silica Flash Column, Eluent of 0˜30% Ethyl acetate/Petroleum ether gradient @80 mL/min). The eluent was concentrated under reduced pressure to give Intermediate 1-5 (2 g, 4.16 mmol, 99.90% yield) as a white solid. LCMS (Method D): Retention time: 0.531 min, [M+H]+=482.1.

### Step 4: Synthesis of Intermediate 1-6.

To a mixture of Intermediate 1-5 (1.36 g, 2.84 mmol, 1 eq) and Intermediate 1-3 (900 mg, 2.84 mmol, 1 eq) in DMSO (10 mL) was added L-proline (65.29 mg, 567.06 μmol, 0.2 eq), K₂CO₃ (1.18 g, 8.51 mmol, 3 eq), and CuI (54.00 mg, 283.53 μmol, 0.1 eq), then the mixture was stirred at 100° C. for 1 hour under N₂. The reaction mixture was diluted with water (150 mL) and extracted with EA (30 mL*2). The combined organic layers were concentrated under reduced pressure to give a residue. The residue was purified by flash silica gel chromatography (ISCO; 40 g SepaFlash Silica Flash Column, Eluent of 0˜60% Ethyl acetate/Petroleum ether gradient @80 mL/min). The eluent was concentrated under reduced pressure to give Intermediate 1-6 (1 g, 1.47 mmol, 52.00% yield, 98.9% purity) as a yellow solid. LCMS (Method D): Retention time: 0.420 min, [M+H]+=671.5.

### Step 5: Synthesis of Intermediate 1-7.

To a mixture of Intermediate 1-6 (800 mg, 1.19 mmol, 1 eq) in MeOH (8 mL) was added Pd(OH)₂ (16.75 mg, 119.25 μmol, 0.1 eq). The suspension was degassed and purged with N₂ 3 times, and then the mixture was degassed and purged with H₂ 3 times, then the mixture was stirred at 40° C. for 1 hour under H₂ atmosphere (15 psi). The reaction mixture was filtered and concentrated under reduced pressure to give Intermediate 1-7 (800 mg, crude) as a colorless oil. LCMS (Method D): Retention time: 0.308 min, [M+H]+=537.3.

### Step 6: Synthesis of Intermediate 1-9.

To a mixture of Intermediate 1-7 (400 mg, 745.29 μmol, 1 eq) and Intermediate 1-8 (330.06 mg, 745.29 μmol, 1 eq) in DMF (4 mL) was added DIEA (288.97 mg, 2.24 mmol, 389.45 μL, 3 eq), then the mixture was stirred at 60° C. for 1 hour. The reaction mixture was diluted with water (100 mL) and extracted with EA (30 mL*2). The combined organic layers were concentrated under reduced pressure to give a residue. The crude product was purified by reversed-phase HPLC (0.1% FA condition). The eluent was concentrated under reduced pressure to remove MeCN and then lyophilized to give Intermediate 1-9 (200 mg, 194.67 μmol, 26.12% yield, 91.8% purity) as a yellow solid. LCMS (Method D): Retention time: 0.356 min, [M+H]+=943.4.

### Step 7: Synthesis of Intermediate 1-10.

A mixture of Intermediate 1-9 (170 mg, 180.25 μmol, 1 eq) in HCl/dioxane (1 mL) was stirred at 20° C. for 10 min. The reaction mixture was concentrated under reduced pressure to give Intermediate 1-10 (150 mg, 170.56 μmol, 94.62% yield, HCl salt) as a white solid. LCMS: Retention time: 0.248 min, [M+H]+=843.6.

### Step 8: Synthesis of Intermediate 1-12.

To a mixture of Intermediate 1-10 (150 mg, 170.56 μmol, 1 eq, HCl salkt) and Intermediate 1-11 (73.42 mg, 341.12 μmol, 2 eq) in DMF (2 mL) was added HOAt (23.21 mg, 170.56 μmol, 23.86 μL, 1 eq), EDCI (98.09 mg, 511.68 μmol, 3 eq), and NMM (86.26 mg, 852.79 μmol, 93.76 μL, 5 eq), then the mixture was stirred at 20° C. for 1 hour. The reaction mixture was diluted with H₂O (50 mL) and extracted with EA (10 mL*3). The combined organic layers were concentrated under reduced pressure to give Intermediate 1-12 (150 mg, crude) as a white solid. LCMS (Method D): Retention time: 0.361 min, [M+H]+=1040.8.

### Step 9: Synthesis of I-313.

A mixture of Intermediate 1-12 (120 mg, 111.45 μmol, 1 eq, HCl salt) in HCl/dioxane (1 mL) was stirred at 20° C. for 10 min. The reaction mixture was concentrated under reduced pressure to give a residue. The crude product was purified by reversed-phase HPLC (0.1% FA condition). The eluent was concentrated under reduced pressure to remove MeCN and then lyophilized to give I-313 (99.18 mg, 98.96 μmol, 88.80% yield, 98.4% purity, FA salt) as a yellow solid. LCMS (Method D): Retention time: 0.262 min, [M+H]+=940.4. SFC: Rt=2.051 min, 2.664 min. ¹H NMR (400 MHz, METHANOL-d4) δ=8.38 (s, 2H), 8.36 (s, 1H), 8.31-8.28 (m, 1H), 7.99-7.92 (m, 1H), 7.91-7.81 (m, 2H), 7.55-7.43 (m, 2H), 7.41-7.34 (m, 1H), 7.32-7.23 (m, 1H), 7.21-7.10 (m, 2H), 7.10-7.03 (m, 1H), 6.57-6.54 (m, 1H), 4.59-4.50 (m, 1H), 4.39 (s, 2H), 4.12-3.93 (m, 4H), 3.84-3.74 (m, 3H), 3.70 (s, 1H), 3.61-3.53 (m, 6H), 3.42 (s, 5H), 3.25-3.17 (m, 1H), 2.88-2.70 (m, 4H), 2.66-2.56 (m, 5H), 2.35 (s, 2H), 2.09-1.95 (m, 3H), 1.92-1.79 (m, 3H), 1.72-1.46 (m, 3H), 0.81-0.73 (m, 4H). ¹⁹F NMR (376 MHz, METHANOL-d4) δ=−120.66.

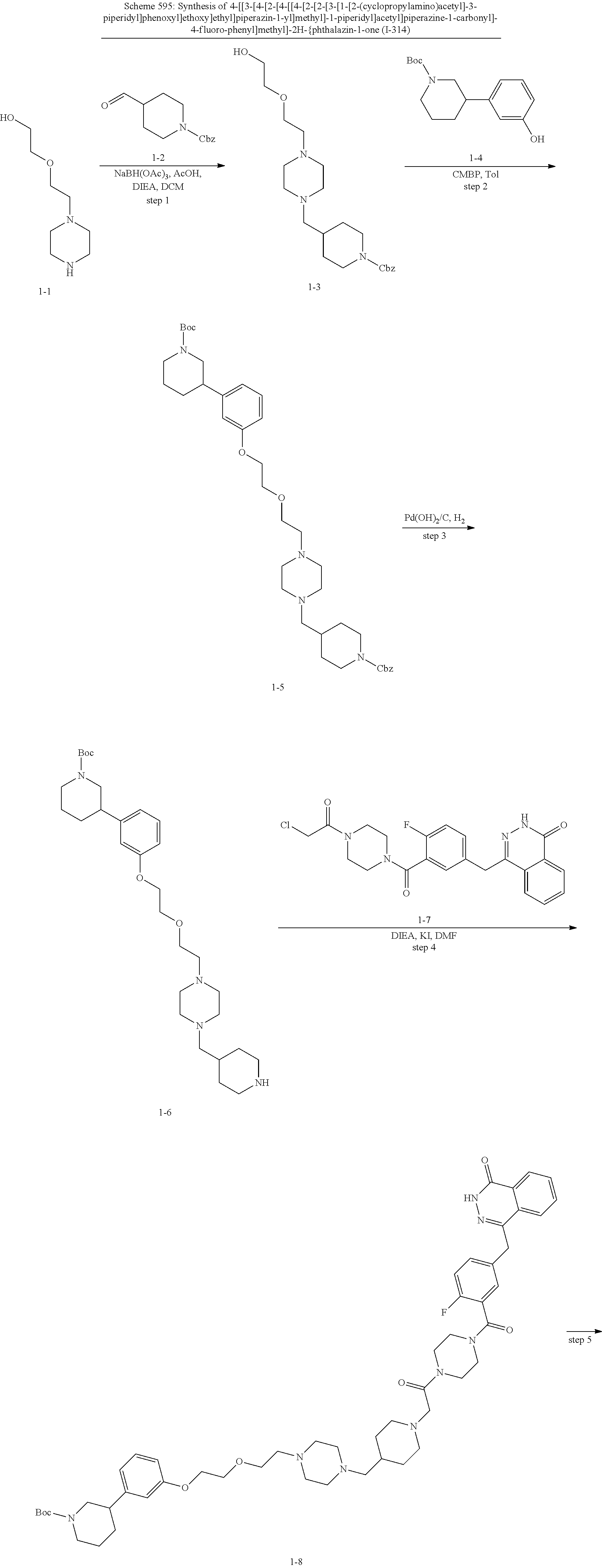

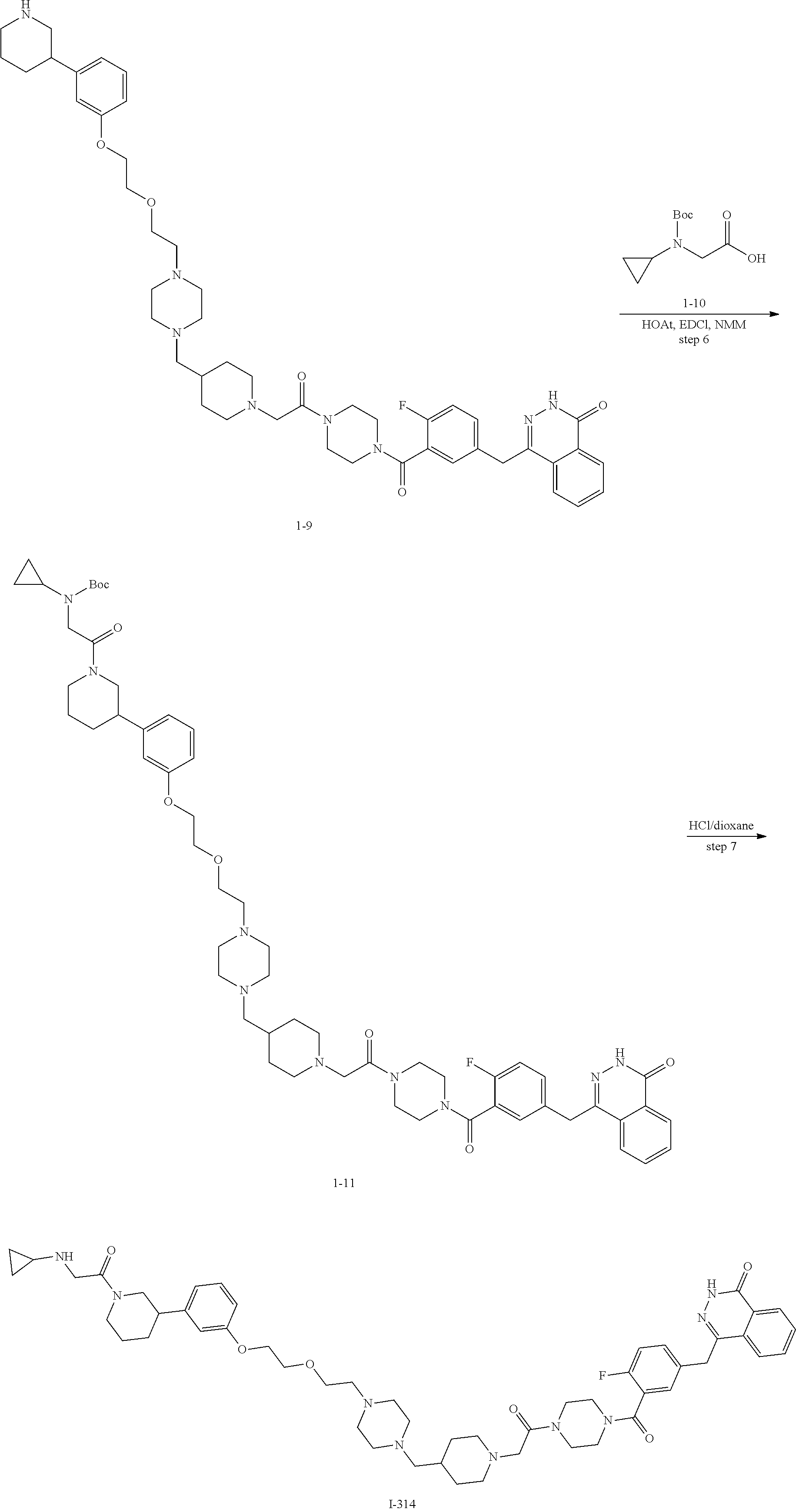 [SMILES: O=C(CN1CCC(CN2CCN(CCOCCOc3cccc(C4CCCNC4)c3)CC2)CC1)N1CCN(C(=O)c2cc(Cc3n[nH]c(=O)c4ccccc34)ccc2F)CC1 ; CC(C)(C)OC(=O)N(CC(=O)O)C1CC1 ; CC(C)(C)OC(=O)N(CC(=O)N1CCCC(c2cccc(OCCOCCN3CCN(CC4CCN(CC(=O)N5CCN(C(=O)c6cc(Cc7n[nH]c(=O)c8ccccc78)ccc6F)CC5)CC4)CC3)c2)C1)C1CC1 ; O=C(CN1CCC(CN2CCN(CCOCCOc3cccc(C4CCCN(C(=O)CNC5CC5)C4)c3)CC2)CC1)N1CCN(C(=O)c2cc(Cc3n[nH]c(=O)c4ccccc34)ccc2F)CC1]

### Step 1: Synthesis of Intermediate 1-3

To a solution of Intermediate 1-1 (2 g, 11.48 mmol, 1.89 mL, 1 eq) and Intermediate 1-2 (2.84 g, 11.48 mmol, 1 eq) in DCM (20 mL) was added NaBH(OAc)₃ (6.08 g, 28.70 mmol, 2.5 eq), AcOH (137.86 mg, 2.30 mmol, 131.42 μL, 0.2 eq) and DIEA (2.97 g, 22.96 mmol, 4.00 mL, 2 eq). The mixture was stirred at 25° C. for 2 hr. The mixture was diluted with aq. NH₄Cl (2 mL), and extracted with EtOAc (2 mL*3). The organic layer was concentrated under vacuum. The crude product was purified by reversed-phase HPLC (0.1% NH₃H₂O condition) and concentrated under vacuum to remove MeCN and dried by lyophilization. Intermediate 1-3 (4 g, 9.86 mmol, 85.93% yield) was obtained as a colorless oil. LCMS (Method G): Rt=0.524 min, [M+H]⁺=406.4.

### Step 2: Synthesis of Intermediate 1-5

To the solution of Intermediate 1-3 (219.32 mg, 540.82 μmol, 1.5 eq) in toluene (2 mL) was added Intermediate 1-4 (0.1 g, 360.55 μmol, 1 eq) and 2-(tributyl-phosphanylidene)acetonitrile (870.18 mg, 3.61 mmol, 10 eq). The mixture was degassed and purged with N₂ 3 times, and then the mixture was stirred at 100° C. for 12 h under N₂ atmosphere. The mixture was concentrated under vacuum. The residue was combined with another lot of material (0.1 g of Intermediate 1-4 starting material) for purification. The crude product was purified by reversed-phase HPLC (0.1% NH₃H₂O condition) and concentrated under vacuum. Intermediate 1-5 (0.4 g, crude) was obtained as a red oil. LCMS (Method G): Rt=0.817 min, [M+H]⁺=665.5.

### Step 3: Synthesis of Intermediate 1-6

A solution of Pd(OH)₂/C (120.00 mg, 170.89 μmol, 20% purity, 2.52e−1 eq) in EtOH (5 mL) was added Intermediate 1-5 (450 mg, 676.82 μmol, 1 eq). The suspension was degassed and purged with H₂ 3 times. The mixture was stirred under H₂ (50 Psi) at 25° C. for 4 hr. The mixture was filtered through a pad of Celite and washed with MeOH (30 mL*3). The filtrate was concentrated under vacuum to give a residue. The residue was used in next step without purification. Intermediate 1-6 (320 mg, 602.93 μmol, 89.08% yield) as a yellow oil was obtained. LCMS (Method E): Rt=0.428 min, [M+H]⁺=531.5.

### Step 4: Synthesis of Intermediate 1-8

To the solution of Intermediate 1-6 (120 mg, 226.10 μmol, 1 eq) in DMF (1 mL) was added Intermediate 1-7 (100.13 mg, 226.10 μmol, 1 eq), DIEA (116.88 mg, 904.40 μmol, 157.53 μL, 4 eq) and KI (150.13 mg, 904.40 μmol, 4 eq), and the mixture was stirred at 25° C. for 1.5 hr. The mixture was diluted with water (3 mL), extracted with EtOAc (3 mL*3), and washed with brine (2 mL*2). The organic layer was concentrated under vacuum. The crude product was purified by reversed-phase HPLC (0.1% FA condition) and concentrated under vacuum to remove MeCN and dried by lyophilization. Intermediate 1-8 (105 mg, 112.04 μmol, 49.55% yield) was obtained as a brown solid. LCMS (Method G): Rt=0.732 min, [M+H]⁺=937.6. ¹H NMR (400 MHz, DMSO-d₆) δ=12.60 (s, 1H), 8.26 (br d, J=7.6 Hz, 1H), 8.20 (s, 1H), 8.00-7.93 (m, 1H), 7.92-7.78 (m, 2H), 7.49-7.33 (m, 2H), 7.27-7.16 (m, 2H), 6.86-6.75 (m, 3H), 4.33 (s, 2H), 4.12-4.03 (m, 2H), 3.95 (br d, J=11.8 Hz, 2H), 3.70 (br d, J=4.0 Hz, 2H), 3.66-3.54 (m, 5H), 3.48 (br d, J=17.9 Hz, 2H), 3.37 (br s, 1H), 3.27-3.04 (m, 4H), 2.91-2.68 (m, 4H), 2.63-2.40 (m, 9H), 2.33 (br s, 3H), 2.16-1.91 (m, 4H), 1.85 (br d, J=12.0 Hz, 1H), 1.74-1.53 (m, 4H), 1.39 (s, 10H), 1.18-0.93 (m, 2H).

### Step 5: Synthesis of Intermediate 1-9

A mixture of Intermediate 1-8 (75 mg, 80.03 μmol, 1 eq) in HCl/dioxane (2 M, 1 mL, 24.99 eq) was stirred at 25° C. for 0.5 hr. The mixture was concentrated under vacuum. The residue was used in the next step without further purification. Intermediate 1-9 (70 mg, crude, HCl salt) was obtained as a brown solid. LCMS: Rt=0.364 min, [M+H]⁺=837.5.

### Step 6: Synthesis of Intermediate 1-11

A mixture of Intermediate 1-9 (70 mg, 80.14 μmol, 1 eq, HCl salt), Intermediate 1-10 (17.25 mg, 80.14 μmol, 1 eq) and HOAt (10.91 mg, 80.14 μmol, 11.21 μL, 1 eq) in DMF (1 mL) was added EDCI (30.72 mg, 160.28 μmol, 2 eq) and NMM (40.53 mg, 400.69 μmol, 44.05 μL, 5 eq). The mixture was stirred at 25° C. for 2 hrs. The mixture was diluted with water (2 mL). The crude product was purified by reversed-phase HPLC (0.10% FA condition) and concentrated under vacuum to give the product (80 mg). The product (70 mg) and was purified by Prep-HPLC (column: Waters Xbridge 150*25 mm*5 um; mobile phase: [water (NH₄HCO₃)-ACN]; gradient: 40%-70% B over 9 min) and dried by lyophilization. Intermediate 1-11 (20 mg, 19.34 μmol, 28.57% yield) as a white solid was obtained. LCMS: Rt=0.469 min, [M+H]⁺=1034.9.

### Step 7: Synthesis of I-314

A mixture of Intermediate 1-11 (20 mg, 19.34 μmol, 1 eq) in HCl/dioxane (2 M, 400.00 μL, 41.37 eq) was stirred at 25° C. for 0.5 hr. The mixture was concentrated by a stream of N₂ and dried by lyophilization to give I-314 (16.7 mg, 16.50 μmol, 85.34% yield, 95.918% purity, HCl salt) as a yellow solid. LCMS (Method E): Rt=0.393 min, [M+H]⁺=934.6. SFC: Rt=1.808 min, 2.378 min.

¹H NMR (400 MHz, METHANOL-d₄) δ=8.37 (d, J=7.6 Hz, 1H), 8.01-7.93 (m, 1H), 7.92-7.80 (m, 2H), 7.55-7.47 (m, 1H), 7.38-7.37 (m, 1H), 7.25-7.24 (m, 1H), 7.19-7.15 (m, 1H), 6.98-6.91 (m, 1H), 6.90-6.81 (m, 2H), 4.57-4.53 (m, 1H), 4.39 (s, 2H), 4.35 (br s, 1H), 4.33-4.28 (m, 1H), 4.25 (br d, J=12.8 Hz, 1H), 4.22-4.18 (m, 2H), 4.01 (br s, 2H), 3.91 (br d, J=3.6 Hz, 5H), 3.86-3.75 (m, 5H), 3.74-3.64 (m, 4H), 3.63-3.50 (m, 5H), 3.44-3.33 (m, 4H), 3.28-3.11 (m, 4H), 3.01-2.86 (m, 1H), 2.85-2.60 (m, 3H), 2.43-2.19 (m, 3H), 2.16-1.97 (m, 2H), 1.94-1.79 (m, 2H), 1.79-1.53 (m, 3H), 1.00-0.87 (m, 4H).

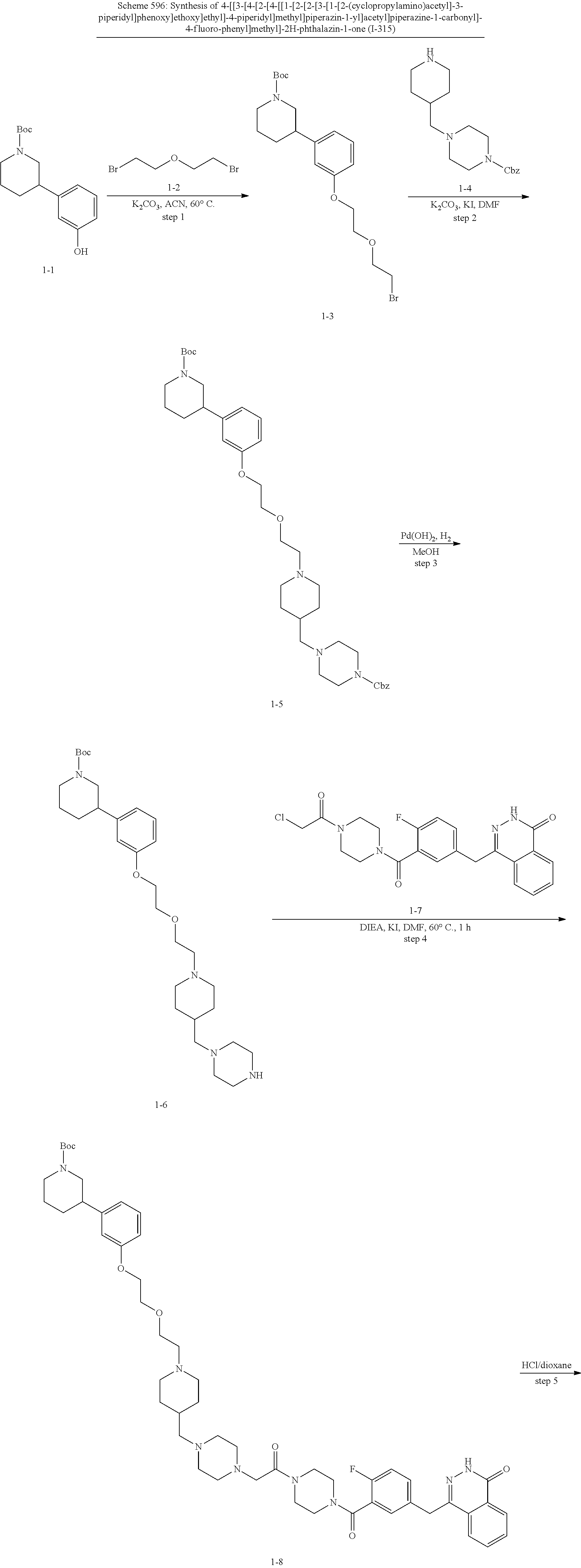

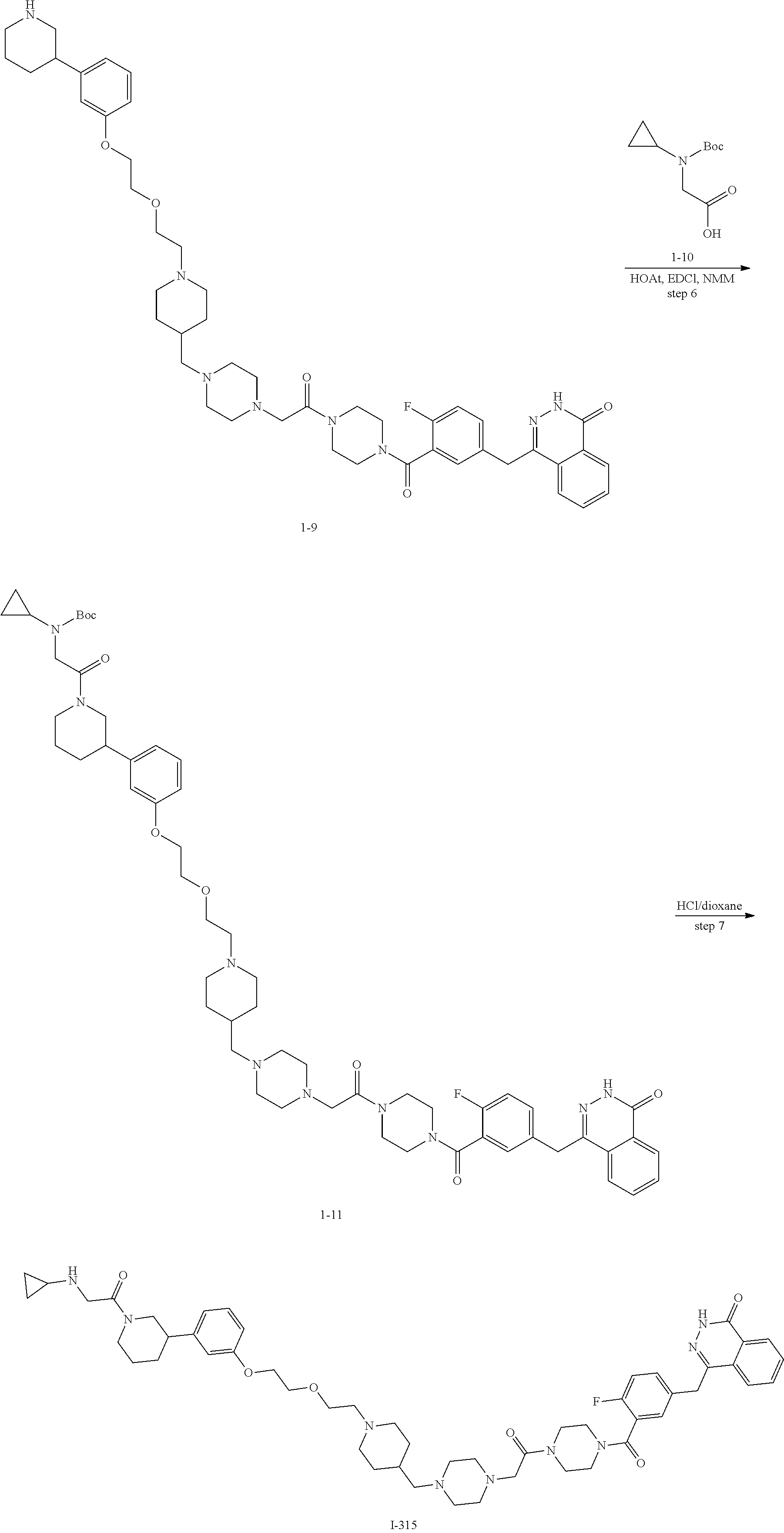 [SMILES: O=C(CN1CCN(CC2CCN(CCOCCOc3cccc(C4CCCNC4)c3)CC2)CC1)N1CCN(C(=O)c2cc(Cc3n[nH]c(=O)c4ccccc34)ccc2F)CC1 ; CC(C)(C)OC(=O)N(CC(=O)O)C1CC1 ; CC(C)(C)OC(=O)N(CC(=O)N1CCCC(c2cccc(OCCOCCN3CCC(CN4CCN(CC(=O)N5CCN(C(=O)c6cc(Cc7n[nH]c(=O)c8ccccc78)ccc6F)CC5)CC4)CC3)c2)C1)C1CC1 ; O=C(CN1CCN(CC2CCN(CCOCCOc3cccc(C4CCCN(C(=O)CNC5CC5)C4)c3)CC2)CC1)N1CCN(C(=O)c2cc(Cc3n[nH]c(=O)c4ccccc34)ccc2F)CC1]

### Step 1: Synthesis of Intermediate 1-3

To a solution of Intermediate 1-1 (500 mg, 1.80 mmol, 1 eq) in ACN (10 mL) was added K₂CO₃ (747.44 mg, 5.41 mmol, 3 eq) and Intermediate 1-2 (627.11 mg, 2.70 mmol, 339.90 μL, 1.5 eq). The mixture was stirred at 60° C. for 12 h. The reaction mixture was quenched by H₂O (20 mL) and extracted with EA (10 mL*3). The combined organic layers were washed with brine (10 mL*3), dried over anhydrous Na₂SO₄, filtered and concentrated under reduced pressure to give a residue. The residue was purified by flash silica gel chromatography (ISCO; 20 g SepaFlash Silica Flash Column, Eluent of 0˜50% ethyl acetate/Petroleum ether gradient @100 mL/min) and concentrated under vacuum. Intermediate 1-3 (570 mg, 1.33 mmol, 73.81% yield) was obtained as a colorless oil. LCMS (Method G): Rt=0.771 min, [M−55]⁺=372.1. ¹H NMR (400 MHz, DMSO-d₆) δ=7.22 (t, J=7.9 Hz, 1H), 6.88-6.77 (m, 3H), 4.16-4.06 (m, 2H), 4.02-3.85 (m, 2H), 3.83-3.76 (m, 4H), 3.65-3.59 (m, 2H), 2.93-2.65 (m, 2H), 2.62-2.53 (m, 1H), 1.92-1.80 (m, 1H), 1.74-1.53 (m, 2H), 1.40 (s, 10H).

### Step 2: Synthesis of Intermediate 1-5

To a solution of Intermediate 1-3 (470 mg, 1.10 mmol, 1 eq) in DMF (5 mL) was added K₂CO₃ (454.92 mg, 3.29 mmol, 3 eq), KI (18.21 mg, 109.72 μmol, 0.1 eq) and Intermediate 1-4 (388.29 mg, 1.10 mmol, 1 eq, HCl salt). The mixture was stirred at 120° C. for 12 h. The mixture was filtered. The filtrate was purified by reversed-phase HPLC (0.1% FA) and concentrated under vacuum to remove ACN and the aqueous phase was adjusted to pH=8 by sat. NaHCO₃ (4 mL) and extracted with DCM (4 mL*3). The combined organic layers were washed with brine (4 mL*3), dried over anhydrous Na₂SO₄, filtered and concentrated under reduced pressure. Intermediate 1-5 (180 mg, 270.73 μmol, 24.67% yield) was obtained as a light yellow oil. LCMS (Method G): Rt=0.885 min, [M+H]⁺=665.5. ¹H NMR (400 MHz, DMSO-d₆) δ=7.41-7.24 (m, 5H), 7.20 (t, J=8.0 Hz, 1H), 6.83-6.77 (m, 3H), 5.06 (s, 2H), 4.08-4.04 (m, 2H), 3.94 (br d, J=12.0 Hz, 2H), 3.72-3.67 (m, 2H), 3.63-3.46 (m, 3H), 2.97-2.57 (m, 5H), 2.47-2.39 (m, 2H), 2.26 (br d, J=4.4 Hz, 4H), 2.08 (d, J=7.2 Hz, 2H), 1.95-1.81 (m, 3H), 1.78-1.52 (m, 5H), 1.39 (s, 13H), 1.18-0.88 (m, 2H).

### Step 3: Synthesis of Intermediate 1-6

To a solution of Pd(OH)₂ (90.00 mg, 128.17 μmol, 20% purity, 4.73e−1 eq) in MeOH (5 mL) was added Intermediate 1-5 (180 mg, 270.73 μmol, 1 eq). The mixture was degassed and purged with N₂ 3 times, and then the mixture was stirred at 25° C. for 1 h under H₂ (15 psi). The mixture was filtered and the filtrate was concentrated under vacuum to give a crude product. The crude was used for the next step directly without purification. Intermediate 1-6 (130 mg, crude) was obtained as a yellow oil. LCMS: Rt=0.423 min, [M+H]⁺=531.5 (Method E).

### Step 4: Synthesis of Intermediate 1-8

To a solution of Intermediate 1-6 (110 mg, 207.26 μmol, 1 eq) in DMF (2 mL) was added KI (34.41 mg, 207.26 μmol, 1 eq), DIEA (107.15 mg, 829.03 μmol, 144.40 μL, 4 eq) and Intermediate 1-7 (73.43 mg, 165.81 μmol, 0.8 eq). The mixture was stirred at 60° C. for 1 h. The mixture was filtered. The filtrate was purified by reversed-phase HPLC (0.1% NH₃·H₂O). Intermediate 1-8 (80 mg, 84.71 μmol, 40.87% yield, 99.232% purity) was obtained as a light yellow solid. LCMS (Method G): Rt=0.836 min, [M+H]⁺=937.6. ¹H NMR (400 MHz, DMSO-d₆) δ=12.59 (s, 1H), 8.26 (d, J=7.8 Hz, 1H), 8.01-7.93 (m, 1H), 7.89 (br t, J=7.1 Hz, 1H), 7.86-7.78 (m, 1H), 7.49-7.33 (m, 2H), 7.28-7.18 (m, 2H), 6.84-6.76 (m, 3H), 4.33 (s, 2H), 4.12-4.03 (m, 2H), 3.95 (br d, J=12.6 Hz, 2H), 3.70 (br d, J=4.1 Hz, 2H), 3.64-3.53 (m, 5H), 3.52-3.43 (m, 3H), 3.19-3.07 (m, 4H), 2.93-2.59 (m, 5H), 2.42-2.18 (m, 7H), 2.12-1.78 (m, 6H), 1.69-1.53 (m, 4H), 1.39 (s, 12H), 1.11-0.99 (m, 2H).

### Step 5: Synthesis of Intermediate 1-9

To a solution of Intermediate 1-8 (80 mg, 85.37 μmol, 1 eq) in DCM (0.5 mL) was added HCl/dioxane (2 M, 1 mL, 23.43 eq). The mixture was stirred at 25° C. for 1 h. The mixture was concentrated under vacuum to give a crude product. The crude was used for the next step directly without purification. Intermediate 1-9 (80 mg, crude, HCl salt) was obtained as a yellow solid. LCMS (Method E): Rt=0.379 min, [M+H]⁺=837.5.

### Step 6: Synthesis of Intermediate 1-11

To a solution of Intermediate 1-9 (70 mg, 80.14 μmol, 1 eq, HCl salt) in DMF (1 mL) was added HOAt (10.91 mg, 80.14 μmol, 11.21 μL, 1 eq), EDCI (30.73 mg, 160.28 μmol, 2 eq), NMM (40.53 mg, 400.69 μmol, 44.05 μL, 5 eq) and Intermediate 1-10 (17.25 mg, 80.14 μmol, 1 eq). The mixture was stirred at 25° C. for 1 h. The mixture was filtered. The filtrate was purified by reversed-phase HPLC (0.1% NH₃·H₂O). Intermediate 1-11 (60 mg, 56.67 μmol, 70.72% yield, 97.69% purity) was obtained as a yellow solid. LCMS (Method G): Rt=0.783 min, [M+H]⁺=1034.7.

### Step 7: Synthesis of I-315

To a solution of Intermediate 1-11 (50 mg, 48.34 μmol, 1 eq) in dioxane (0.5 mL) was added HCl/dioxane (2 M, 1 mL, 41.37 eq). The mixture was stirred at 25° C. for 1 h. To the mixture was added 40 ml of H₂O and the mixture was freeze-dried to give a crude I-315 (27.63 mg, HCl salt) as an off-white solid. LCMS (Method E): Rt=0.391 min, [M+H]⁺=934.6. SFC: Rt=4.076 min, 5.945 min. H NMR (400 MHz, METHANOL-d₄) δ=8.37 (d, J=7.6 Hz, 1H), 8.00-7.92 (m, 1H), 7.92-7.79 (m, 2H), 7.48-7.51 (m, 1H), 7.41-7.34 (m, 1H), 7.29-7.23 (m, 1H), 7.15-7.18 (m, 1H), 6.95-6.82 (m, 3H), 4.49 (br d, J=15.2 Hz, 2H), 4.39 (s, 2H), 4.27-4.17 (m, 3H), 3.96-3.89 (m, 4H), 3.85-3.68 (m, 12H), 3.56 (br s, 2H), 3.43-3.37 (m, 4H), 3.25-3.19 (m, 3H), 3.16-3.10 (m, 3H), 2.88 (s, 2H), 2.85-2.78 (m, 2H), 2.70-2.62 (m, 1H), 2.27-2.18 (m, 2H), 2.03 (br d, J=12.8 Hz, 1H), 1.92-1.78 (m, 3H), 1.74-1.56 (m, 3H), 1.34-1.29 (m, 1H), 1.11-1.07 (m, 1H), 0.99-0.88 (m, 4H).

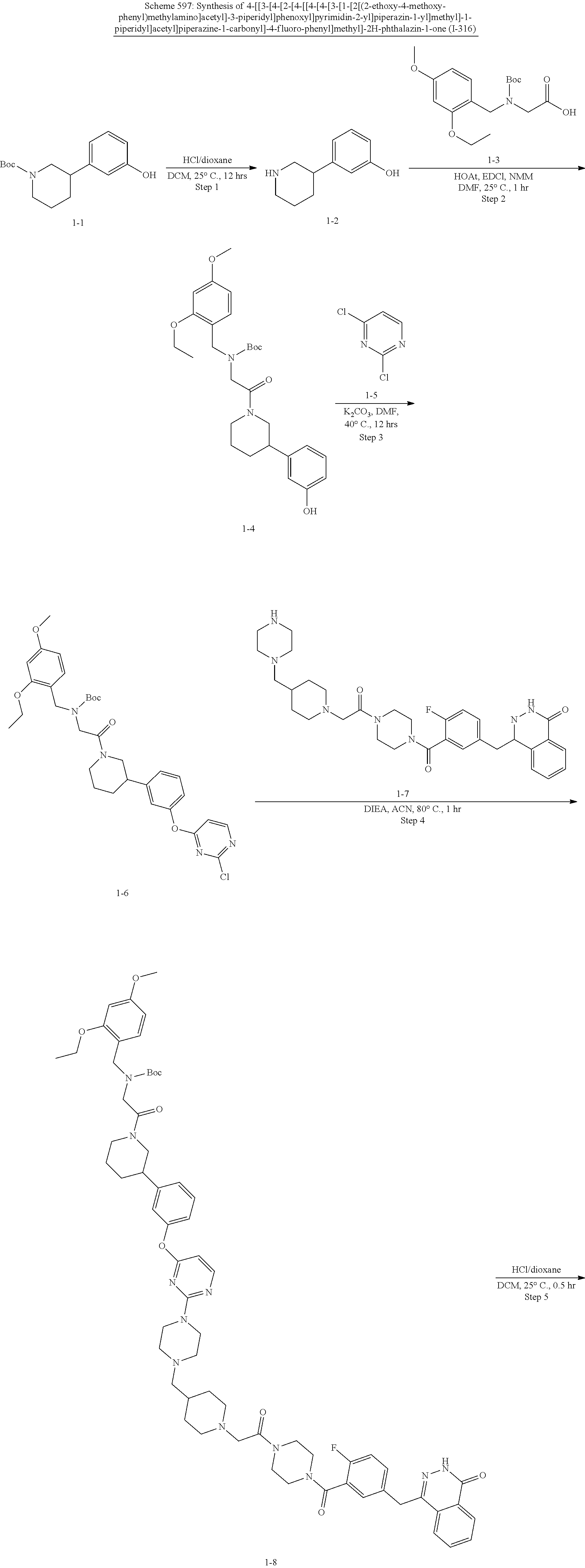

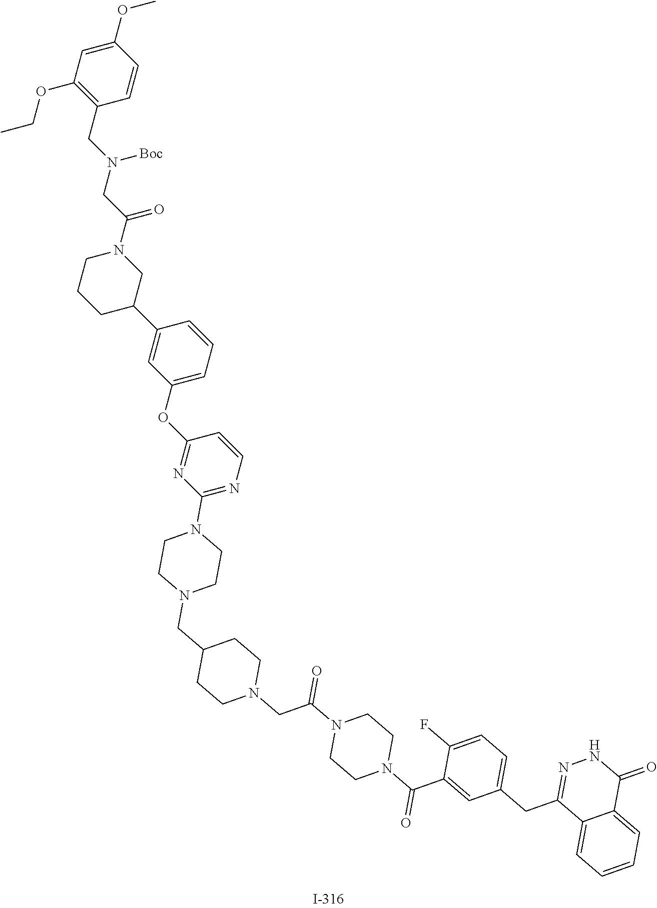 [SMILES: CCOc1cc(OC)ccc1CN(CC(=O)N1CCCC(c2cccc(Oc3ccnc(N4CCN(CC5CCN(CC(=O)N6CCN(C(=O)c7cc(Cc8n[nH]c(=O)c9ccccc89)ccc7F)CC6)CC5)CC4)n3)c2)C1)C(=O)OC(C)(C)C]

### Step 1: Synthesis of Intermediate 1-2

To a solution of Intermediate 1-1 (500 mg, 1.80 mmol, 1 eq) in CH₂Cl₂ (2 mL) and HCl/dioxane (2 M, 3 mL, 3.33 eq) was stirred at 25° C. for 12 hrs. The mixture was concentrated under reduced pressure to give Intermediate 1-2 (400 mg, crude, HCl salt) as a yellow solid, which was used in the next step without further purification. LCMS (Method H): Rt: 0.346 min, [M+H]+=177.9.

### Step 2: Synthesis of Intermediate 1-4

To a solution of Intermediate 1-3 (158.81 mg, 467.94 μmol, 1 eq) in DMF (1.5 mL) was added EDCI (269.11 mg, 1.40 mmol, 3 eq), HOAt (63.69 mg, 467.94 μmol, 65.46 μL, 1 eq), NMM (236.65 mg, 2.34 mmol, 257.23 μL, 5 eq) and Intermediate 1-2 (100 mg, 467.94 μmol, 1 eq, HCl salt). The mixture was stirred at 25° C. for 1 hr. The mixture was filtered. The filtrate was purified by flash silica gel chromatography (ISCO; 20 g SepaFlash Silica Flash Column, Eluent of 0˜25% Ethyl acetate/Petroleum ether gradient @40 mL/min). The eluent was concentrated under reduced pressure to give Intermediate 1-4 (200 mg, 391.89 μmol, 83.75% yield, 97.7% purity) as a yellow oil. LCMS (Method D): Rt: 0.522 min, [M+H]+=499.2. ¹H NMR (400 MHz, METHANOL-d4) δ=7.16-7.07 (m, 2H), 6.76-6.60 (m, 3H), 6.54-6.44 (m, 2H), 4.60-4.54 (m, 4H), 4.53-4.48 (m, 1H), 4.42-4.37 (m, 1H), 4.16-4.08 (m, 1H), 4.07-3.98 (m, 3H), 3.77 (s, 3H), 2.62-2.43 (m, 1H), 2.01-1.95 (m, 1H), 1.86-1.78 (m, 1H), 1.78-1.68 (m, 1H), 1.63-1.52 (m, 1H), 1.49-1.43 (m, 9H), 1.42-1.34 (m, 3H).

### Step 3: Synthesis of Intermediate 1-6

To a solution of Intermediate 1-4 (200 mg, 401.12 μmol, 1 eq) in DMF (2 mL) was added Intermediate 1-5 (59.76 mg, 401.12 μmol, 1 eq) and K₂CO₃ (166.31 mg, 1.20 mmol, 3 eq). The mixture was stirred at 40° C. for 12 hr. The reaction mixture diluted with H₂O (3 mL) and extracted with EA (3 mL*2). The combined organic layers were washed with brine (2 mL*1), dried over Na₂SO₄, filtered and concentrated under reduced pressure to give a residue. The residue was purified by flash silica gel chromatography (ISCO; 12 g SepaFlash Silica Flash Column, Eluent of 0˜45% Ethyl acetate/Petroleum ether gradient @60 mL/min). The eluent was concentrated under reduced pressure to give Intermediate 1-6 (120 mg, 194.59 μmol, 48.51% yield, 99.1% purity) as a colorless oil. LCMS (Method D): Rt: 0.534 min, [M+H]+=611.2. ¹H NMR (400 MHz, METHANOL-d4) δ=8.49 (d, J=5.6 Hz, 1H), 7.47-7.37 (m, 1H), 7.29-7.18 (m, 1H), 7.16-7.02 (m, 3H), 7.00-6.93 (m, 1H), 6.53-6.42 (m, 2H), 4.58-4.52 (m, 1H), 4.47-4.33 (m, 2H), 4.18-4.08 (m, 1H), 4.07-3.96 (m, 3H), 3.83-3.71 (m, 4H), 3.17-3.01 (m, 1H), 2.80-2.60 (m, 2H), 2.08-2.00 (m, 1H), 1.88-1.75 (m, 2H), 1.66-1.53 (m, 1H), 1.45 (s, 9H), 1.42-1.32 (m, 3H).

### Step 4: Synthesis of Intermediate 1-8

To a solution of Intermediate 1-6 (60 mg, 98.18 μmol, 1 eq) in ACN (1 mL) was added DIEA (38.07 mg, 294.54 μmol, 51.30 μL, 3 eq) and Intermediate 1-7 (57.90 mg, 98.18 μmol, 1 eq). The mixture was stirred at 80° C. for 1 hr. The mixture was filtered. The filtrate was purified by reversed-phase HPLC (0.1% FA condition). The eluent was concentrated under reduced pressure to remove ACN and then lyophilized to give Intermediate 1-8 (65 mg, 55.82 μmol, 56.86% yield, 100% purity) as a white solid. LCMS (Method D): Rt: 0.416 min, [M+H]+=1164.8.

### Step 5: Synthesis of I-316

To a solution of Intermediate 1-8 (60 mg, 51.53 μmol, 1 eq) in CH₂Cl₂ (0.6 mL) and HCl/dioxane (2 M, 0.3 mL, 11.64 eq) was stirred at 25° C. for 0.5 hr. The mixture was filtered. The filtrate was purified by reversed-phase HPLC (0.1% FA condition). The eluent was concentrated under reduced pressure to remove ACN and then lyophilized to give I-316 (14.52 mg, 13.08 μmol, 25.38% yield, 100% purity, FA salt) as a white solid. LCMS (Method D): Rt: 0.319 min, [M+H]+=1064.2. ¹H NMR (400 MHz, METHANOL-d4) δ=8.48 (s, 1H), 8.39-8.34 (m, 1H), 8.20-8.14 (m, 1H), 7.98-7.92 (m, 1H), 7.90-7.80 (m, 2H), 7.54-7.46 (m, 1H), 7.43-7.33 (m, 2H), 7.31-7.24 (m, 1H), 7.21-7.14 (m, 2H), 7.11-7.01 (m, 2H), 6.63-6.58 (m, 1H), 6.58-6.53 (m, 1H), 6.17-6.10 (m, 1H), 4.58-4.48 (m, 1H), 4.39 (s, 2H), 4.22-4.09 (m, 4H), 4.06-3.92 (m, 2H), 3.83-3.78 (m, 5H), 3.78-3.57 (m, 10H), 3.56-3.50 (m, 1H), 3.45 (s, 1H), 3.35 (s, 1H), 3.29-3.21 (m, 2H), 3.20-3.09 (m, 1H), 2.75 (s, 1H), 2.73-2.54 (m, 3H), 2.48 (s, 4H), 2.35-2.27 (m, 2H), 2.09-1.99 (m, 1H), 1.95-1.84 (m, 3H), 1.84-1.73 (m, 2H), 1.68-1.53 (m, 1H), 1.51-1.36 (m, 5H)¹⁹F NMR (376 MHz, METHANOL-d4) δ=−120.715. SFC: Rt: 2.872 min, 3.512 min.

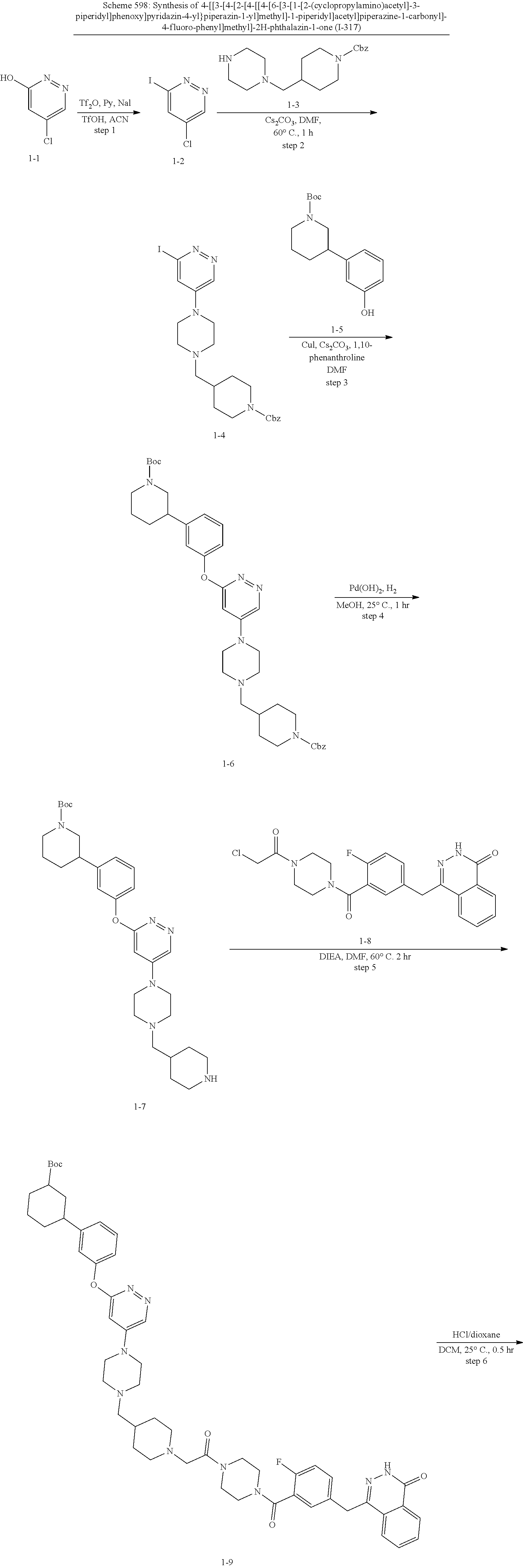

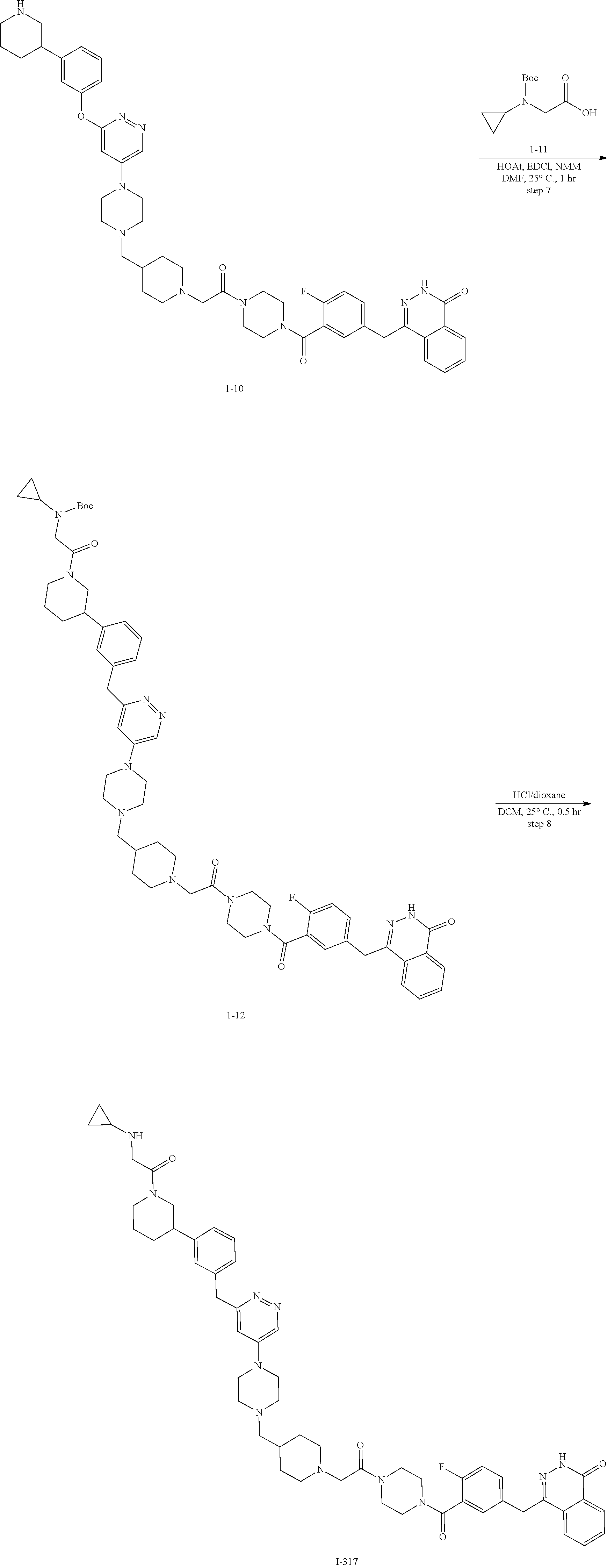 [SMILES: O=C(CN1CCC(CN2CCN(c3cnnc(Oc4cccc(C5CCCNC5)c4)c3)CC2)CC1)N1CCN(C(=O)c2cc(Cc3n[nH]c(=O)c4ccccc34)ccc2F)CC1 ; CC(C)(C)OC(=O)N(CC(=O)O)C1CC1 ; CC(C)(C)OC(=O)N(CC(=O)N1CCCC(c2cccc(Cc3cc(N4CCN(CC5CCN(CC(=O)N6CCN(C(=O)c7cc(Cc8n[nH]c(=O)c9ccccc89)ccc7F)CC6)CC5)CC4)cnn3)c2)C1)C1CC1 ; O=C(CN1CCC(CN2CCN(c3cnnc(Cc4cccc(C5CCCN(C(=O)CNC6CC6)C5)c4)c3)CC2)CC1)N1CCN(C(=O)c2cc(Cc3n[nH]c(=O)c4ccccc34)ccc2F)CC1]

### Step 1: Synthesis of Intermediate 1-2

A solution of pyridine (2.91 g, 36.77 mmol, 2.97 mL, 1.2 eq) and Intermediate 1-1 (4 g, 30.64 mmol, 1 eq) in ACN (40 mL) was cooled to 0° C. and Tf₂O (7.35 g, 26.05 mmol, 4.30 mL, 0.85 eq) was added dropwise. The mixture was stirred for 30 min, then NaI (4.59 g, 30.64 mmol, 1 eq) was added in one portion. TfOH (5.06 g, 33.71 mmol, 2.98 mL, 1.1 eq) was added dropwise and the mixture was stirred for 20 min at 0° C. The pH of the reaction mixture was basified to 7-8 with aq. NaHCO₃ and the mixture was washed with aq·Na₂SO₃, then the mixture was extracted with EA (50 mL*3). The combined organic layer was dried over Na₂SO₄, filtered and concentrated in vacuum to give a residue. The residue was purified by column chromatography (SiO₂, PE/EA=1/0 to 0/1, Rf=0.4, PE:EA=5:1) to give product and the eluent was concentrated in vacuum. Intermediate 1-2 (2.3 g, 8.42 mmol, 27.47% yield, 88% purity) was obtained as a yellow solid. LCMS (Method D): Rt=0.299 min, [M+H]+=241.0.

### Step 2: Synthesis of Intermediate 1-4

To a solution of Intermediate 1-2 (454.46 mg, 1.89 mmol, 1 eq) and Intermediate 1-3 (600 mg, 1.89 mmol, 1 eq) in DMF (6 mL) was added Cs₂CO₃ (1.85 g, 5.67 mmol, 3 eq). The mixture was stirred at 60° C. for 1 hr. The mixture was washed with water (10 mL) and extracted with EA (20 mL*3). The combined organic layers were dried over sodium sulfate, filtered and concentrated under reduced pressure to give a residue. The residue was purified by flash silica gel chromatography (ISCO; 40 g SepaFlash Silica Flash Column, Eluent of 0˜100% MeOH/EA @60 mL/min) and then the eluent was concentrated in vacuum to give product. Intermediate 1-4 (900 mg, 1.57 mmol, 83.10% yield, 91% purity) was obtained as a yellow gum. LCMS (Method D): Rt=0.282 min, [M+H]+=522.1. ¹H NMR (400 MHz, METHANOL-d4) δ=8.83 (d, J=2.8 Hz, 1H), 7.38-7.33 (m, 5H), 7.31-7.30 (m, 1H), 5.11 (s, 2H), 4.14 (d, J=13.2 Hz, 2H), 3.51-3.45 (m, 4H), 2.85 (d, J=9.2 Hz, 2H), 2.56-2.51 (m, 4H), 2.25 (d, J=6.6 Hz, 2H), 1.80 (d, J=10.4 Hz, 3H), 1.15-1.05 (m, 2H).

### Step 3: Synthesis of Intermediate 1-6

To a solution of CuI (7.31 mg, 38.36 μmol, 0.2 eq) and 1,10-phenanthroline (10.37 mg, 57.54 mol, 0.3 eq) in DMF (1 mL) was stirred at 25° C. for 0.5 h, then Intermediate 1-4 (100 mg, 191.79 μmol, 1 eq), Cs₂CO₃ (124.98 mg, 383.59 μmol, 2 eq) and Intermediate 1-5 (53.20 mg, 191.79 μmol, 1 eq) were added. The mixture was degassed and purged with N₂ 3 times. The mixture was stirred at 110° C. for 2 hr. The mixture was washed with water (3 mL) and extracted with EA (5 mL*3). The combined organic layers were dried over sodium sulfate, filtered and concentrated under reduced pressure to give a residue. The residue was purified by flash silica gel chromatography (ISCO; 80 g SepaFlash Silica Flash Column, Eluent of 0˜100% MeOH/EA @100 mL/min) and then the eluent was concentrated in vacuum to give product. Intermediate 1-6 (90 mg, 123.43 μmol, 64.35% yield, 92% purity) was obtained as a yellow oil. LCMS (Method D): Rt=0.406 min, [M+H]+=671.4. ¹H NMR (400 MHz, METHANOL-d4) δ=8.68 (s, 1H), 7.38-7.29 (m, 6H), 7.13 (d, J=7.6 Hz, 1H), 7.04 (s, 1H), 6.98-6.96 (m, 1H), 6.47 (s, 1H), 5.11 (s, 2H), 4.19-4.13 (m, 2H), 4.05 (s, 1H), 3.51-3.44 (m, 4H), 2.91-2.77 (m, 4H), 2.71-2.65 (m, 1H), 2.58-2.53 (m, 4H), 2.26 (d, J=6.6 Hz, 2H), 1.98 (d, J=4.0 Hz, 1H), 1.85-1.72 (m, 5H), 1.72-1.64 (m, 1H), 1.55-1.52 (m, 1H), 1.45 (s, 9H), 1.14-1.06 (m, 2H).

### Step 4: Synthesis of Intermediate 1-7

To a solution of Intermediate 1-6 (80 mg, 119.25 μmol, 1 eq) in MeOH (1 mL) was added Pd(OH)₂/C (40 mg, 20% purity) under N₂ atmosphere. The suspension was degassed and purged with H₂ 3 times. The mixture was stirred under H₂ (15 Psi) at 25° C. for 1 hr. The resulting mixture was filtered, and the filtrate was concentrated under reduced pressure. The crude product was used in the next step without further purification. Intermediate 1-7 (55 mg, 102.48 μmol, 85.93% yield) was obtained as a yellow gum. LCMS (Method D): Rt=0.293 min, [M+H]+=537.3. ¹H NMR (400 MHz, METHANOL-d4) δ=8.68 (d, J=2.4 Hz, 1H), 7.40-7.32 (m, 1H), 7.14 (d, J=7.2 Hz, 1H), 7.04 (s, 1H), 6.98-6.96 (m, 1H), 6.48 (d, J=1.6 Hz, 1H), 4.12-4.02 (m, 2H), 3.50-3.45 (m, 4H), 3.10 (d, J=12.4 Hz, 2H), 2.90-2.80 (m, 2H), 2.72-2.69 (m, 1H), 2.66-2.62 (m, 1H), 2.58-2.53 (m, 4H), 2.27-2.24 (m, 2H), 2.01 (d, J=12.0 Hz, 1H), 1.89-1.62 (m, 6H), 1.59-1.53 (m, 1H), 1.45 (s, 9H), 1.24-1.14 (m, 2H).

### Step 5: Synthesis of Intermediate 1-9

To a solution of Intermediate 1-7 (50 mg, 93.16 μmol, 1 eq) and Intermediate 1-8 (41.26 mg, 93.16 μmol, 1 eq) in ACN (0.5 mL) was added DIEA (36.12 mg, 279.48 μmol, 48.68 μL, 3 eq). The mixture was stirred at 60° C. for 2 hr. The reaction mixture was concentrated in vacuum. The residue was purified by prep-HPLC (FA condition), the eluent was concentrated under reduced pressure to remove ACN and lyophilized to give product. Intermediate 1-9 (45 mg, 44.37 μmol, 47.63% yield, 93% purity) was obtained as a white solid. LCMS (Method D): Rt=0.331 min, [M+H]+=943.4. ¹H NMR (400 MHz, METHANOL-d4) δ=8.68 (s, 1H), 8.37 (d, J=8.0 Hz, 1H), 7.94 (d, J=6.4 Hz, 1H), 7.91-7.81 (m, 2H), 7.50 (s, 1H), 7.36-7.34 (m, 2H), 7.20-7.10 (m, 2H), 7.04 (s, 1H), 6.98 (d, J=8.4 Hz, 1H), 6.48 (s, 1H), 4.39 (s, 2H), 4.14-4.02 (m, 2H), 3.95 (s, 1H), 3.86-3.69 (m, 4H), 3.57 (d, J=16.4 Hz, 2H), 3.48 (s, 4H), 3.41-3.33 (m, 4H), 2.88-2.64 (m, 5H), 2.58 (s, 4H), 2.36-2.28 (m, 2H), 1.99 (s, 3H), 1.84-1.66 (m, 3H), 1.62-1.49 (m, 2H), 1.45 (s, 9H), 1.41-1.34 (m, 2H). ¹⁹F NMR (376 MHz, MeOD-d4) δ=−120.710.

### Step 6: Synthesis of Intermediate 1-10

To a solution of Intermediate 1-9 (40 mg, 42.41 μmol, 1 eq) in DCM (0.5 mL) was added HCl/dioxane (2 M, 0.2 mL). The mixture was stirred at 25° C. for 0.5 hr. The reaction mixture was concentrated in vacuum. The crude product was used in the next step without further purification. Intermediate 1-10 (40 mg, crude, HCl salt) was obtained as a white solid. LCMS (Method D): Rt=0.252 min, [M+H]+=843.5.

### Step 7: Synthesis of Intermediate 1-12

To a solution of Intermediate 1-11 (9.79 mg, 45.48 μmol, 1 eq) in DMF (0.5 mL) was added EDCI (26.16 mg, 136.45 μmol, 3 eq), HOAt (6.19 mg, 45.48 μmol, 6.36 μL, 1 eq) and NMM (23.00 mg, 227.41 μmol, 25.00 μL, 5 eq). Then Intermediate 1-10 (40 mg, 45.48 μmol, 1 eq, HCl salt) was added. The mixture was stirred at 25° C. for 1 hr. The resulting mixture was filtered, and the filtrate was concentrated under reduced pressure. The residue was purified by prep-HPLC (0.1% FA condition) and the eluent was concentrated under reduced pressure to remove ACN and lyophilized to give product. Intermediate 1-12 (30 mg, 26.24 μmol, 57.70% yield, 91% purity) was obtained as a yellow oil. LCMS (Method D): Rt=0.379 min, [M+H]+=1040.6.

### Step 8: Synthesis of I-317

To a solution of Intermediate 1-12 (20 mg, 19.23 μmol, 1 eq) in DCM (0.2 mL) was added HCl/dioxane (2 M, 0.1 mL). The mixture was stirred at 25° C. for 0.5 hr. The reaction mixture was concentrated in vacuum. To the resulting mixture was added H₂O (1 ml) and it was lyophilized to give product. I-317 (16.59 mg, 15.91 μmol, 82.77% yield, 93.679% purity, HCl salt) was obtained as a white solid. LCMS (Method D): Rt=0.254 min, [M+H]+=940.5. SFC: Rt=1.719 min, 2.169 min. ¹H NMR (400 MHz, METHANOL-d4) δ=8.85 (s, 1H), 8.37 (d, J=7.6 Hz, 1H), 7.99-7.93 (m, 1H), 7.91-7.81 (m, 2H), 7.56-7.48 (m, 2H), 7.44 (d, J=6.4 Hz, 1H), 7.40-7.33 (m, 2H), 7.25 (d, J=7.2 Hz, 1H), 7.17-7.15 (m, 1H), 6.66-6.55 (m, 1H), 4.56 (d, J=10.0 Hz, 1H), 4.46-4.34 (m, 5H), 4.32-4.20 (m, 3H), 3.88-3.77 (m, 5H), 3.69 (d, J=13.2 Hz, 3H), 3.57-3.51 (m, 2H), 3.47-3.34 (m, 6H), 3.28-3.17 (m, 5H), 2.90-2.72 (m, 3H), 2.38-2.37 (m, 1H), 2.24 (d, J=15.2 Hz, 2H), 2.09 (d, J=12.0 Hz, 2H), 1.95-1.84 (m, 2H), 1.79-1.58 (m, 3H), 0.98-0.87 (m, 4H). ¹⁹F NMR (376 MHz, MeOD-d4) δ=−120.747.

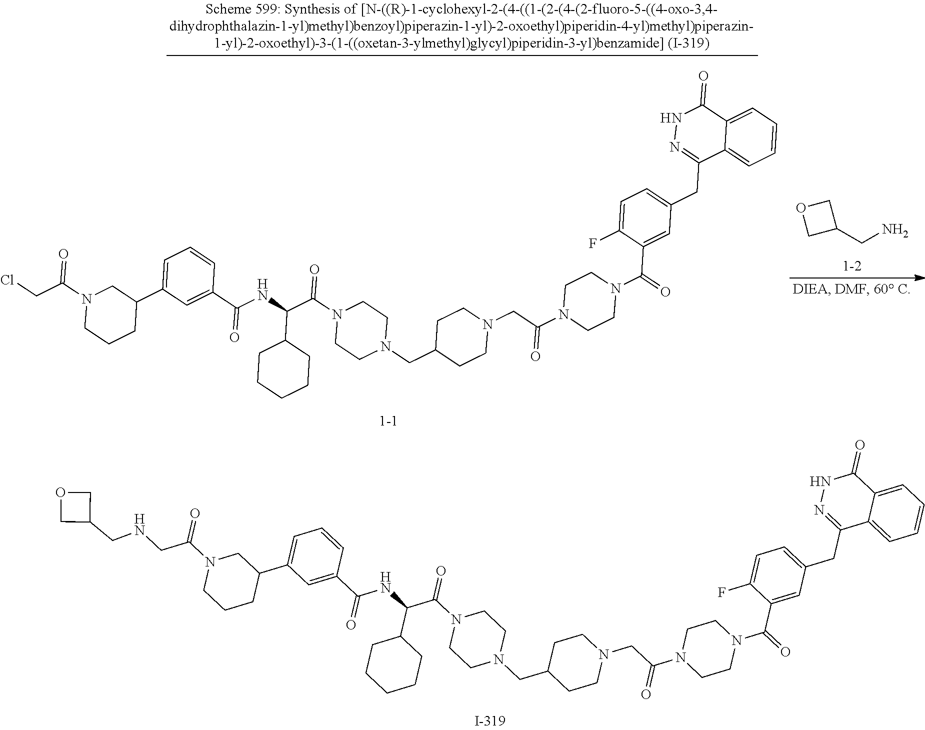 [SMILES: O=C(N[C@@H](C(=O)N1CCN(CC2CCN(CC(=O)N3CCN(C(=O)c4cc(Cc5n[nH]c(=O)c6ccccc56)ccc4F)CC3)CC2)CC1)C1CCCCC1)c1cccc(C2CCCN(C(=O)CCl)C2)c1 ; NCC1COC1 ; O=C(N[C@@H](C(=O)N1CCN(CC2CCN(CC(=O)N3CCN(C(=O)c4cc(Cc5n[nH]c(=O)c6ccccc56)ccc4F)CC3)CC2)CC1)C1CCCCC1)c1cccc(C2CCCN(C(=O)CNCC3COC3)C2)c1]

To a solution of Intermediate 1-1 (50 mg, 50.37 μmol, 1 eq) and Intermediate 1-2 (5.27 mg, 60.45 μmol, 1.2 eq) in DMF (0.5 mL) was added DIEA (19.53 mg, 151.12 μmol, 26.32 μL, 3 eq). The mixture was stirred at 60° C. for 0.5 hr. The reaction mixture was diluted with H₂O (2 mL) and extracted with EA (2 mL*2). The combined organic layers were washed with NaCl (aq) (2 mL*2), dried over Na₂SO₄, filtered and concentrated under reduced pressure to give a residue. The residue was purified by prep-HPLC (column: Phenomenex luna C18 150*25 mm*10 um; mobile phase: [water (FA)-ACN]; gradient: 1%-31% B over 10 min) and the eluent was concentrated to remove ACN and lyophilized to give product. I-319 (14.14 mg, 12.93 μmol, 25.67% yield, 99.599% purity, FA salt) was obtained as a white solid. LCMS (Method D): Retention time: 0.318 min, [M+H]⁺=1043.6. SFC: Retention time: 3.158 min, 3.518 min. ¹H NMR (400 MHz, METHANOL-d₄) δ=8.45 (d, J=6.4 Hz, 1H), 8.37 (d, J=8.4 Hz, 1H), 8.00-7.92 (m, 1H), 7.92-7.80 (m, 2H), 7.79-7.69 (m, 2H), 7.55-7.41 (m, 3H), 7.39-7.35 (m, 1H), 7.19-7.15 (m, 1H), 4.58 (s, 3H), 4.45 (d, J=8.8 Hz, 2H), 4.39 (s, 2H), 3.94-3.85 (m, 2H), 3.84-3.78 (m, 2H), 3.78-3.67 (m, 6H), 3.66-3.56 (m, 3H), 3.55-3.52 (m, 1H), 3.50-3.45 (m, 1H), 3.35 (s, 1H), 3.24-3.12 (m, 5H), 3.01-2.69 (m, 3H), 2.59-2.40 (m, 6H), 2.28-2.24 (m, 2H), 2.13-2.01 (m, 1H), 1.96-1.64 (m, 13H), 1.44-1.19 (m, 6H), 1.16-1.06 (m, 2H). ¹⁹F NMR (377 MHz, METHANOL-d₄) δ=−120.792.

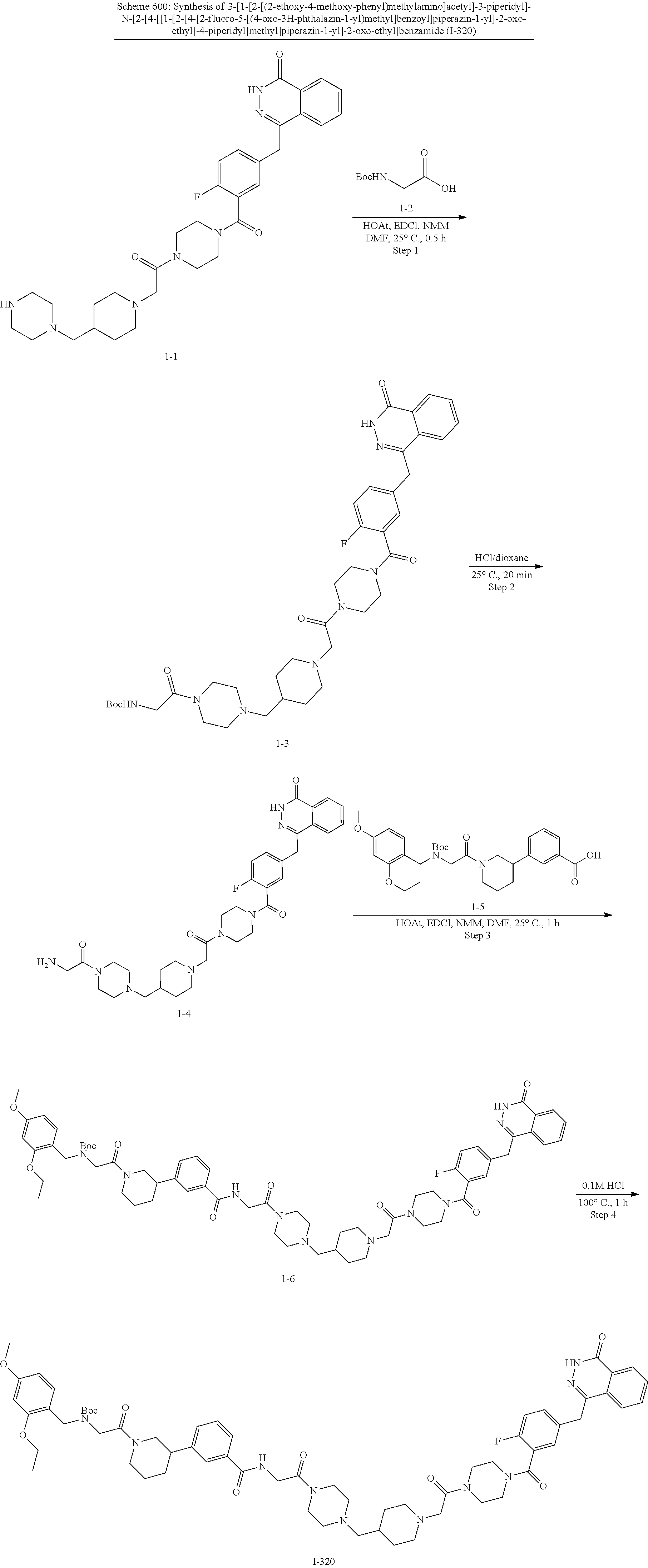

### Step 1: Synthesis of Intermediate 1-3

To a solution of Intermediate 1-1 (200 mg, 319.41 μmol, 1 eq, HCl salt) and Intermediate 1-2 (83.93 mg, 479.11 μmol, 1.5 eq) in DMF (1.5 mL) was added EDCI (183.69 mg, 958.22 μmol, 3 eq), HOAt (43.47 mg, 319.41 μmol, 44.68 μL, 1 eq), and NMM (161.53 mg, 1.60 mmol, 175.58 μL, 5 eq). The mixture was stirred at 25° C. for 0.5 hr. The mixture was diluted with water (5 mL) and extracted with DCM (5 mL*3). The combined organic layer was dried over Na₂SO₄, filtered and concentrated in vacuum to give a residue. The residue was purified by prep-HPLC (column: CD07-Daisogel SP-100-8-ODS-PK 150*25*10 um; mobile phase: [water (NH₄HCO₃)-ACN]; gradient: 20%-50% B over 10 min). Then the eluent was concentrated in vacuum and lyophilized. Intermediate 1-3 (130 mg, 168.84 μmol, 52.86% yield, 97% purity) was obtained as a colorless oil. LCMS (Method D): Retention time=0.237 min, [M+H]+=747.3.

### Step 2: Synthesis of Intermediate 1-4

To a solution of Intermediate 1-3 (100 mg, 133.89 μmol, 1 eq) in DCM (0.2 mL) was added HCl/dioxane (2 M, 1.00 mL, 14.94 eq). The mixture was stirred at 25° C. for 20 min. The reaction mixture was concentrated in vacuum to give a residue. The residue was used in the next step directly without further purification. Intermediate 1-4 (100 mg, crude, HCl salt) was obtained as a white solid. LCMS (Method D): Retention time=0.220 min, [M+H]+=647.4.

### Step 3: Synthesis of Intermediate 1-6

To a solution of Intermediate 1-4 (100 mg, 146.37 μmol, 1 eq, HCl salt) and Intermediate 1-5 (84.79 mg, 161.00 μmol, 1.1 eq) in DMF (0.5 mL) was added EDCI (84.18 mg, 439.10 μmol, 3 eq), HOAt (19.92 mg, 146.37 μmol, 20.47 μL, 1 eq), and NMM (74.02 mg, 731.84 μmol, 80.46 μL, 5 eq). The mixture was stirred at 25° C. for 1 hr. The mixture was diluted with water (5 mL) and extracted with DCM (5 mL*3). The combined organic layer was dried over Na₂SO₄, filtered and concentrated in vacuum to give a residue. The residue was purified by column chromatography (SiO₂, PE:EA=5:1 to EA:MeOH=0:1), then the organic liquid was concentrated in vacuum to give a residue. Intermediate 1-6 (150 mg, 124.64 mol, 85.15% yield, 96% purity) was obtained as a yellow solid. LCMS (Method D): Retention time=0.369 min, [M+H]+=1155.6.

### Step 4: Synthesis of I-320

A solution of Intermediate 1-6 (50 mg, 43.28 μmol, 1 eq) in HCl (0.1 M, 0.5 mL, 1.16 eq) was stirred at 100° C. for 1 hr. The reaction mixture was concentrated in vacuum to give a residue. The residue was purified by prep-HPLC (column: Phenomenex luna C18 150*25 mm*10 um; mobile phase: [water (HCl)-ACN]; gradient: 8%-38% B over 10 min). Then the eluent was concentrated in vacuum and lyophilized. I-320 (16.08 mg, 14.64 μmol, 33.83% yield, 99.406% purity, HCl salt) was obtained as a white solid. LCMS (Method D): Retention time=0.281 min, [M+H]+=1055.5. SFC: Retention time=0.852 min, 0.929 min. ¹H NMR (400 MHz, METHANOL-d₄) δ=8.36 (d, J=7.6 Hz, 1H), 7.99-7.93 (m, 1H), 7.89 (d, J=6.4 Hz, 1H), 7.86 (s, 1H), 7.84-7.81 (m, 1H), 7.76 (d, J=5.6 Hz, 1H), 7.53-7.44 (m, 3H), 7.39 (t, J=6.4 Hz, 1H), 7.31 (t, J=8.0 Hz, 1H), 7.17 (t, J=9.6 Hz, 1H), 6.63-6.55 (m, 2H), 4.61 (s, 1H), 4.57-4.43 (m, 2H), 4.39 (s, 3H), 4.32 (s, 1H), 4.26-4.21 (m, 3H), 4.19-4.14 (m, 2H), 4.13-4.05 (m, 2H), 4.04-3.94 (m, 1H), 3.84 (s, 2H), 3.81 (d, J=4.8 Hz, 3H), 3.78 (d, J=4.0 Hz, 1H), 3.73 (s, 3H), 3.70-3.69 (m, 1H), 3.55 (d, J=4.0 Hz, 2H), 3.45 (s, 1H), 3.40 (s, 2H), 3.35 (s, 2H), 3.31 (s, 3H), 3.26 (s, 1H), 3.23 (d, J=4.4 Hz, 2H), 3.19 (s, 1H), 3.16-3.07 (m, 1H), 2.92-2.83 (m, 1H), 2.80-2.68 (m, 1H), 2.39 (s, 1H), 2.23 (d, J=13.6 Hz, 2H), 2.08-2.03 (m, 1H), 1.88 (t, J=10.8 Hz, 2H), 1.83-1.70 (m, 2H), 1.68-1.56 (m, 1H), 1.46-1.42 (m, 3H). ¹⁹F NMR (377 MHz, METHANOL-d₄) δ=−120.697.

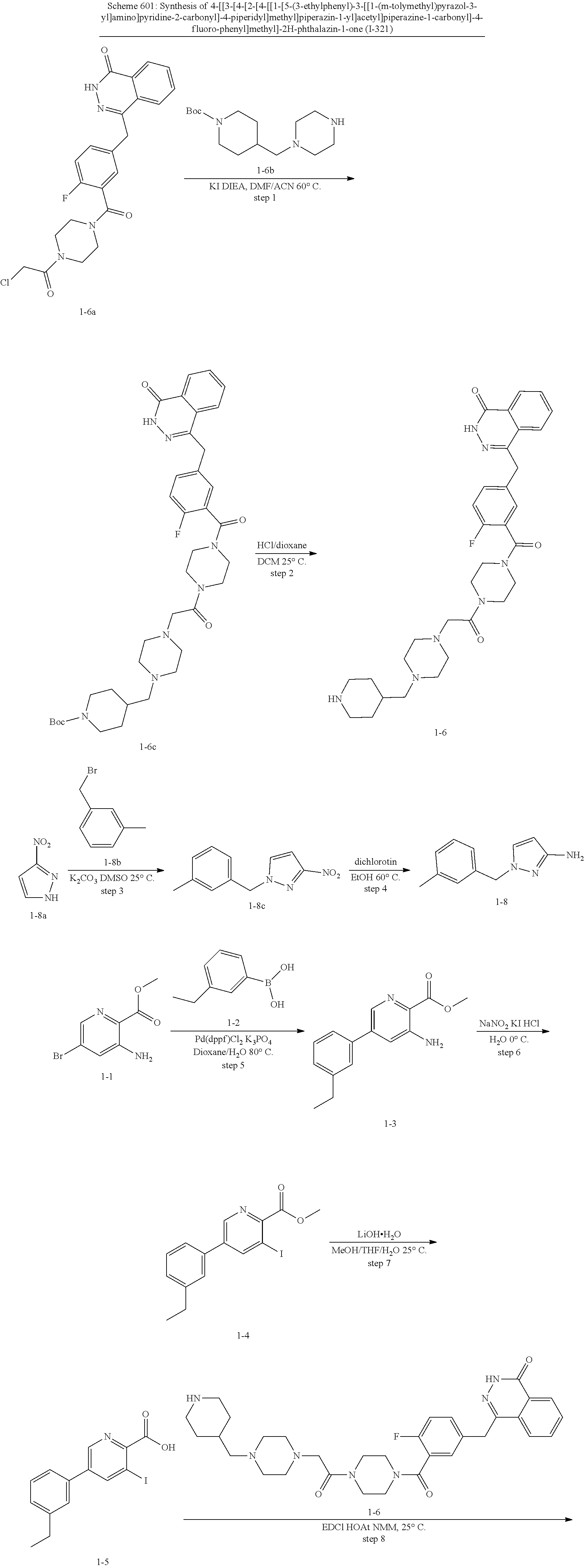

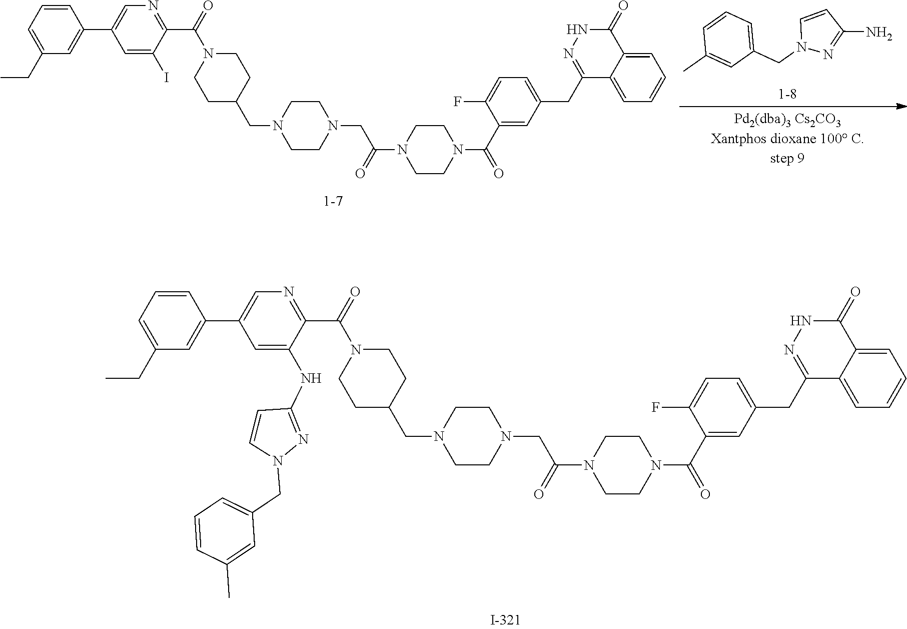

### Step 1: Synthesis of Intermediate 1-6c

To a solution of Intermediate 1-6a (200 mg, 451.60 μmol, 1 eq) and Intermediate 1-6b (140.79 mg, 496.76 μmol, 1.1 eq) in DMF (0.2 mL) and ACN (0.2 mL) was added KI (74.97 mg, 451.60 μmol, 1 eq) and DIEA (233.46 mg, 1.81 mmol, 314.63 μL, 4 eq). The mixture was stirred at 60° C. for 0.5 hr. The reaction mixture was quenched by addition of water (5 mL) at 25° C., and then the mixture was extracted with EA (5 mL*3). Then the organics were dried over Na₂SO₄, filtered and concentrated under reduced pressure to give a residue. The residue was purified by reversed phase chromatography (26.8*125 mm, 40 g of XB—C18, 20-40 μm, 120 Å) Mobile phase: A for H₂O (0.1% NH₃·H₂O v/v) and B for acetonitrile; Gradient: B 30%-50% in 30 min; Flow rate: 60 ml/min; Column temperature: R.T. Wavelength: 220 nm/254 nm). Then eluent was concentrated to remove organic solvents. Intermediate 1-6c (280 mg, 396.77 μmol, 87.86% yield, 97.75% purity) was obtained as colourless oil. LCMS (Method E): Rt=0.424 min, [M+H]⁺=690.4.

### Step 2: Synthesis of Intermediate 1-6

To a solution of Intermediate 1-6c (240 mg, 347.92 μmol, 1 eq) in DCM (2 mL) was added HCl/dioxane (4 M, 2.40 mL, 27.59 eq). The mixture was stirred at 25° C. for 1 hr. The reaction mixture was concentrated under reduced pressure to give a residue. The crude product was used in the next step without further purification. Intermediate 1-6 (220 mg, crude) was obtained as a yellow solid. LCMS (Method E): Rt=0.363 min, [M+H]⁺=590.4. ¹H NMR (400 MHz, DMSO-d₆) δ=12.63 (d, J=3.6 Hz, 1H), 8.25 (d, J=8.0 Hz, 1H), 8.00-7.76 (m, 3H), 7.49-7.32 (m, 2H), 7.24 (m, 1H), 4.33 (s, 4H), 3.85-3.65 (m, 8H), 3.48-3.08 (m, 12H), 2.87-2.78 (m, 2H), 2.16 (s, 1H), 2.04 (d, J=13.2 Hz, 2H), 1.90 (s, 1H), 1.47 (d, J=11.2 Hz, 2H).

### Step 3: Synthesis of Intermediate 1-8c

To a solution of Intermediate 1-8a (0.3 g, 2.65 mmol, 1 eq) in DMSO (5 mL) was added K₂CO₃ (733.35 mg, 5.31 mmol, 2 eq) and Intermediate 1-8b (490.99 mg, 2.65 mmol, 358.38 μL, 1 eq) at 25° C. The mixture was stirred at 25° C. for 4 hr. The reaction mixture was quenched by addition of water (5 mL) at 25° C., and then the mixture was extracted with EA (5 mL*3), dried over Na₂SO₄, filtered and concentrated under reduced pressure to give a residue. The residue was purified by flash silica gel chromatography (ISCO; 20 g SepaFlash Silica Flash Column, Eluent of 0˜60% Ethyl acetate/Petroleum ether gradient @40 mL/min). Intermediate 1-8c (0.52 g, 2.39 mmol, 90.23% yield) was obtained as a colorless oil.

### Step 4: Synthesis of Intermediate 1-8

To a solution of Intermediate 1-8c (500 mg, 2.30 mmol, 1 eq) in EtOH (5 mL) was added dichlorotin (4.36 g, 23.02 mmol, 597.06 μL, 10 eq). The mixture was stirred at 80° C. for 1 hr. The reaction mixture was concentrated under reduced pressure to give a residue. The residue was purified by reversed phase chromatography (26.8*125 mm, 80 g of XB—C18, 20-40 m, 120 Å) Mobile phase: A for H₂O (0.1% NH₃·H₂O v/v) and B for acetonitrile; Gradient: B 30%-50% in 30 min; Flow rate: 60 ml/min; Column temperature: R.T. Wavelength: 220 nm/254 nm), after purification, then concentrated to remove organic solvents. Intermediate 1-8 (380 mg, 1.96 mmol, 85.00% yield, 96.4% purity) was obtained as colourless oil. LCMS (Method E): Rt=0.408 min, [M+H]⁺=188.3. ¹H NMR (400 MHz, DMSO-d₆) δ=7.41 (d, J=2.4 Hz, 1H), 7.23-7.16 (m, 1H), 7.06 (d, J=7.6 Hz, 1H), 7.01-6.94 (m, 2H), 5.41 (d, J=2.4 Hz, 1H), 4.97 (s, 2H), 4.58 (s, 2H), 2.26 (s, 3H).

### Step 5: Synthesis of Intermediate 1-3

To a solution of Intermediate 1-1 (2 g, 8.66 mmol, 1 eq) and Intermediate 1-2 (1.56 g, 10.39 mmol, 1.2 eq) in dioxane (16 mL) and H₂O (4 mL) was added K₃PO₄ (5.51 g, 25.97 mmol, 3 eq), the reaction mixture was purged with N₂ three times then Pd(dppf)Cl₂ (633.38 mg, 865.63 μmol, 0.1 eq) was added to the mixture and the reaction mixture was purged with N₂ three times then it was stirred at 80° C. for 2 h. The reaction mixture was quenched by addition of H₂O (20 mL), extracted with EA (20 mL*3), dried over Na₂SO₄, filtered and concentrated under reduced pressure to give a residue. The residue was purified by flash silica gel chromatography (ISCO; 80 g SepaFlash Silica Flash Column, Eluent of 0˜50% Ethyl acetate/Petroleum ether gradient @60 mL/min). The eluent was concentrated to give Intermediate 1-3 (2.2 g, 8.58 mmol, 99.16% yield) as a yellow solid. LCMS (Method E): Rt=0.471 min, [M+H]⁺=257.2. ¹H NMR (400 MHz, CHLOROFORM-d) δ=8.30 (d, J=2.0 Hz, 1H), 7.40 (d, J=3.2 Hz, 3H), 7.31-7.27 (m, 1H), 7.21 (d, J=2.0 Hz, 1H), 5.83 (br s, 2H), 4.00 (s, 3H), 2.73 (q, J=7.6 Hz, 2H), 1.29 (t, J=7.6 Hz, 3H).

### Step 6: Synthesis of Intermediate 1-4

To a solution of Intermediate 1-3 (2 g, 7.80 mmol, 1 eq) in HCl (20 mL) was added NaNO₂ (1.08 g, 15.61 mmol, 2 eq) in H₂O (2 mL), and the mixture was stirred at 0° C. for 0.5 h. KI (6.48 g, 39.02 mmol, 5 eq) in H₂O (8 mL) was added. The mixture was stirred at 0° C. for 2.5 h. The reaction was diluted with H₂O (10 mL) and extracted with ethyl acetate 15 mL (5 mL*3). The organic phase was washed with saturated aqueous NaCl (10 mL), dried over Na₂SO₄, filtered and concentrated under reduced pressure to give a residue. The residue was purified by flash silica gel chromatography (ISCO; 40 g SepaFlash Silica Flash Column, Eluent of 0˜100% Ethyl acetate/Petroleum ether gradient @60 mL/min). The eluent was concentrated to afford Intermediate 1-4 (1.6 g, 4.28 mmol, 54.79% yield, 98.12% purity) as a white gum. LCMS (Method E): Rt=0.598 min, [M+H]⁺=368.1. ¹H NMR (400 MHz, METHANOL-d₄) δ=8.79-8.74 (m, 1H), 8.56 (d, J=2.0 Hz, 1H), 7.54-7.37 (m, 3H), 7.34-7.28 (m, 1H), 3.98 (s, 3H), 2.72-2.70 m, 2H), 1.27-1.20 (m, 3H).

### Step 7: Synthesis of Intermediate 1-5

To a solution of Intermediate 1-4 (200 mg, 544.69 μmol, 1 eq) in MeOH (0.6 mL), THF (0.6 mL), H₂O (0.6 mL) was added LiOH·H₂O (68.57 mg, 1.63 mmol, 3 eq). The mixture was stirred at 25° C. for 2 hr. The reaction mixture was concentrated under reduced pressure to give a residue. The crude product was used in the next step without further purification. Intermediate 1-5 (200 mg, crude, lithium salt) was obtained as a white solid. LCMS (Method E): Rt=0.536 min, [M+H]⁺=354.0.

### Step 8: Synthesis of Intermediate 1-7

To a solution of Intermediate 1-5 (100 mg, 277.70 μmol, 9.81e−1 eq, Li salt), Intermediate 1-6 (166.98 mg, 283.16 μmol, 1 eq), in DMF (1 mL) was added EDCI (162.85 mg, 849.49 μmol, 3 eq), NMM (229.13 mg, 2.27 mmol, 249.05 μL, 8 eq) and HOAt (38.54 mg, 283.16 μmol, 39.61 μL, 1 eq). The mixture was stirred at 25° C. for 1 hr. The reaction mixture was quenched by addition of water (5 mL) at 25° C., and then it was extracted with EA (5 mL*3). Then the organic phase was dried over Na₂SO₄, filtered and concentrated under reduced pressure to give a residue. The residue was purified by reversed phase chromatography (26.8*125 mm, 40 g of XB—C18, 20-40 m, 120 Å) Mobile phase: A for H₂O (0.1% NH₃·H₂O v/v) and B for acetonitrile; Gradient: B 30%-50% in 30 min; Flow rate: 60 ml/min; Column temperature: R.T. Wavelength: 220 nm/254 nm). The eluent was concentrated to remove organic solvents. Intermediate 1-7 (250 mg, 251.39 μmol, 88.78% yield, 93% purity) was obtained as a yellow solid. LCMS (Method E): Rt=0.482 min, [M+H]⁺=925.3.

### Step 9: Synthesis of I-321

To a solution of Intermediate 1-7 (98.79 mg, 106.81 μmol, 1 eq) and Intermediate 1-8 (20 mg, 106.81 μmol, 1 eq) in dioxane (1 mL) was added Cs₂CO₃ (52.20 mg, 160.22 μmol, 1.5 eq), (1E,4E)-1,5-diphenylpenta-1,4-dien-3-one;palladium (19.56 mg, 21.36 μmol, 0.2 eq) and (5-diphenylphosphanyl-9,9-dimethyl-xanthen-4-yl)-diphenyl-phosphane (30.90 mg, 53.41 μmol, 0.5 eq). The mixture was stirred at 100° C. for 3 hr. The reaction mixture was quenched by addition of water (5 mL) at 25° C., and then extracted with EA (5 mL*3). Then the organics were dried over Na₂SO₄, filtered and concentrated under reduced pressure to give a residue. The residue was purified by prep-HPLC (column: Phenomenex luna C18 150*25 mm*10 um; mobile phase: [water (FA)-ACN]; gradient: 28%-58% B over 9 min). The eluent was concentrated and lyophilized to afford the desired product. I-321 (14 mg, 12.46 μmol, 11.66% yield, 97.7% purity, TFA salt) was obtained as a yellow solid. LCMS (Method E): Rt=0.530 min, [M+H]⁺=984.7. ¹H NMR (400 MHz, METHANOL-d₄) δ=8.41 (d, J=2.0 Hz, 1H), 8.36 (m, 1H), 8.22 (d, J=2.0 Hz, 1H), 7.97-7.91 (m, 1H), 7.89-7.78 (m, 2H), 7.61 (d, J=2.4 Hz, 1H), 7.49 (d, J=5.2 Hz, 1H), 7.42 (s, 1H), 7.40-7.29 (m, 3H), 7.26 (d, J=7.2 Hz, 1H), 7.21-7.13 (m, 2H), 7.10-7.02 (m, 3H), 6.03 (d, J=2.4 Hz, 1H), 5.20 (s, 2H), 4.69 (d, J=11.2 Hz, 1H), 4.37 (s, 2H), 3.90-3.60 (m, 5H), 3.51 (s, 2H), 3.40 (s, 1H), 3.32 (s, 2H), 3.30 (s, 2H), 3.14 (m, 1H), 2.90 (m, 1H), 2.83-2.53 (m, 9H), 2.42 (d, J=7.2 Hz, 2H), 2.23 (s, 3H), 1.91 (d, J=8.0 Hz, 2H), 1.82-1.61 (m, 1H), 1.38-1.27 (m, 2H), 1.24 (m, 3H).

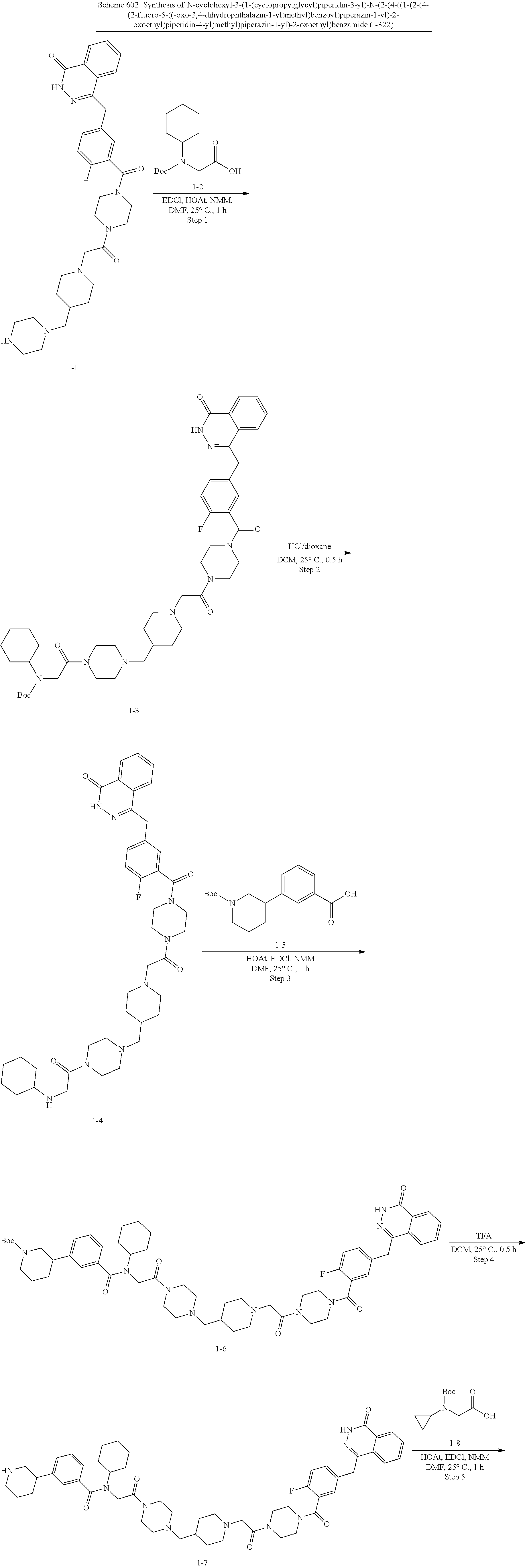

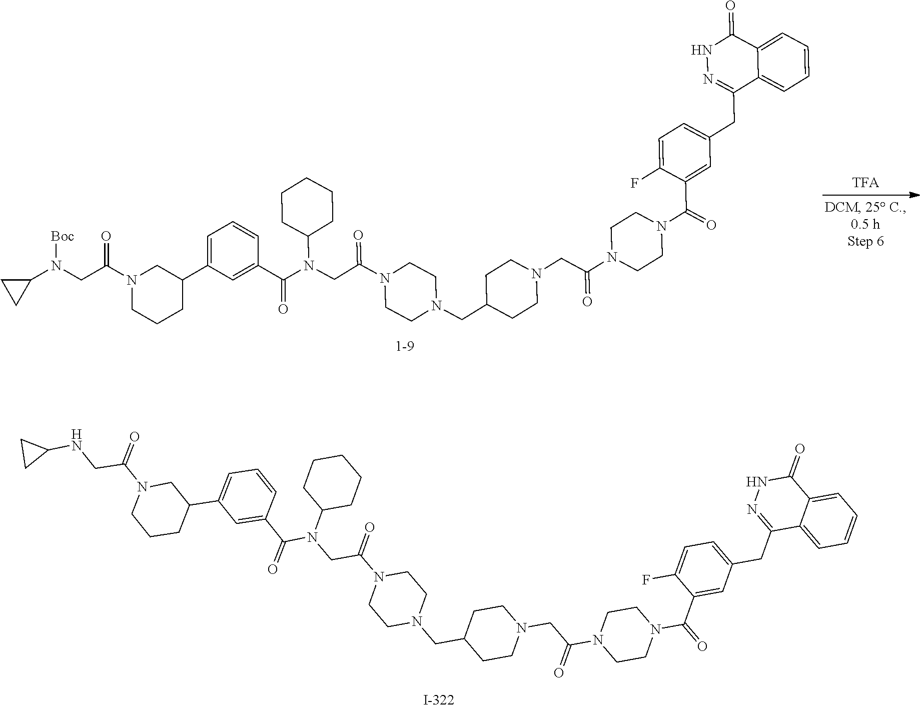 [SMILES: CC(C)(C)OC(=O)N(CC(=O)N1CCCC(c2cccc(C(=O)N(CC(=O)N3CCN(CC4CCN(CC(=O)N5CCN(C(=O)c6cc(Cc7n[nH]c(=O)c8ccccc78)ccc6F)CC5)CC4)CC3)C3CCCCC3)c2)C1)C1CC1 ; O=C(CN1CCC(CN2CCN(C(=O)CN(C(=O)c3cccc(C4CCCN(C(=O)CNC5CC5)C4)c3)C3CCCCC3)CC2)CC1)N1CCN(C(=O)c2cc(Cc3n[nH]c(=O)c4ccccc34)ccc2F)CC1]

### Step 1: Synthesis of Intermediate 1-3

To a solution of Intermediate 1-1 (0.6 g, 1.02 mmol, 1 eq) and Intermediate 1-2 (314.18 mg, 1.22 mmol, 1.2 eq) in DMF (6 mL) was added EDCI (390.10 mg, 2.03 mmol, 2 eq), HOAt (69.24 mg, 508.73 μmol, 71.17 μL, 0.5 eq) and NMM (514.57 mg, 5.09 mmol, 559.31 μL, 5 eq). The mixture was stirred at 25° C. for 1 hr. The reaction mixture was concentrated under reduced pressure to give crude product. The crude product was purified by reversed-phase HPLC (0.1% FA condition) and evaporated to give the product. Intermediate 1-3 (535 mg, 632.16 μmol, 62.13% yield, 97.956% purity) was obtained as a white solid. LCMS (Method D): Rt=0.300 min, [M+H]⁺=829.5.

### Step 2: Synthesis of Intermediate 1-4

A mixture of Intermediate 1-3 (520 mg, 627.25 μmol, 1 eq) in DCM (2.5 mL) was added HCl/dioxane (2 M, 2.5 mL, 7.97 eq), and the mixture was stirred at 25° C. for 0.5 hr. The reaction mixture was concentrated under reduced pressure to give a crude product. The crude product was used in the next step. Intermediate 1-4 (450 mg, 581.49 μmol, 92.70% yield, 98.900% purity, HCl salt) was obtained as a white solid. LCMS (Method D): Rt=0.209 min, [M+H]⁺=729.4.

### Step 3: Synthesis of Intermediate 1-6

A solution of Intermediate 1-4 (450 mg, 587.96 μmol, 1 eq, HCl salt) and Intermediate 1-5 (215.45 mg, 705.55 μmol, 1.2 eq) in DMF (4.5 mL) was added NMM (297.35 mg, 2.94 mmol, 323.21 μL, 5 eq), EDCI (225.43 mg, 1.18 mmol, 2 eq) and HOAt (40.01 mg, 293.98 μmol, 41.12 μL, 0.5 eq) at 25° C., then the mixture was stirred at 25° C. for 1 hr. The reaction mixture was concentrated under reduced pressure to give a crude product. The crude product was purified by reversed-phase HPLC (0.1% FA condition) and lyophilized to give the product. Intermediate 1-6 (300 mg, 270.34 μmol, 45.98% yield, 91.577% purity) was obtained as a white solid. LCMS (Method D): Rt=0.422 min, [M+H]⁺=1016.6.

### Step 4: Synthesis of Intermediate 1-7

To a solution of Intermediate 1-6 (300 mg, 295.20 μmol, 1 eq) in DCM (3 mL) was added TFA (921.00 mg, 8.08 mmol, 600.00 μL, 27.36 eq). The mixture was stirred at 25° C. for 0.5 hr. The reaction mixture was concentrated under reduced pressure to give crude product. The crude product was used in the next step. Intermediate 1-7 (304 mg, 295.10 μmol, 99.97% yield, TFA salt) was obtained as a light-yellow liquid. LCMS (Method D): Rt=0.298 min, [M+H]⁺=916.5.

### Step 5: Synthesis of Intermediate 1-9

To a solution of Intermediate 1-7 (300 mg, 291.22 μmol, 1 eq, TFA salt) and Intermediate 1-8 (75.22 mg, 349.46 μmol, 1.2 eq) in DMF (3 mL) was added HOAt (19.82 mg, 145.61 μmol, 20.37 μL, 0.5 eq), EDCI (111.65 mg, 582.44 μmol, 2 eq) and NMM (147.28 mg, 1.46 mmol, 160.09 μL, 5 eq). The mixture was stirred at 25° C. for 1 hr. The reaction mixture was concentrated under reduced pressure to give a crude product. The crude product was purified by reversed-phase HPLC (0.1% FA condition) and lyophilized to give the product. Intermediate 1-9 (130 mg, 100.71 μmol, 34.58% yield, 86.252% purity) was obtained as a light-yellow solid. LCMS (Method D): Rt=0.436 min, [M+H]⁺=1113.7.

### Step 6: Synthesis of I-322

A mixture of Intermediate 1-9 (30 mg, 26.95 μmol, 1 eq) and TFA (92.10 mg, 807.73 μmol, 60.00 L, 29.98 eq) in DCM (0.3 mL) was stirred at 25° C. for 0.5 hr. The reaction mixture was concentrated under reduced pressure to give a crude product. The crude product was purified by reversed-phase HPLC (0.1% FA condition) and lyophilized to give the product. I-322 (11.42 mg, 9.94 μmol, 36.90% yield, 98.144% purity, TFA salt) was obtained as a colorless gum. LCMS (Method D): Rt=0.312 min, [M+H]⁺=1013.5. SFC: Rt=3.343 min, 3.630 min. ¹H NMR (400 MHz, METHANOL-d₄) δ=8.37 (d, J=7.6 Hz, 1H), 7.99-7.92 (m, 1H), 7.91-7.81 (m, 2H), 7.52 (d, J=2.8 Hz, 1H), 7.48 (d, J=7.2 Hz, 1H), 7.46-7.43 (m, 1H), 7.43 (s, 1H), 7.38-7.30 (m, 2H), 7.18-7.18 (m, 1H), 7.18 (t, J=9.2 Hz, 1H), 4.57 (d, J=11.6 Hz, 1H), 4.39 (s, 2H), 4.33 (d, J=1.2 Hz, 2H), 4.32-4.26 (m, 2H), 4.23 (s, 1H), 4.21-4.14 (m, 1H), 4.12-3.87 (m, 3H), 3.87-3.75 (m, 4H), 3.72 (s, 3H), 3.55 (d, J=2.8 Hz, 2H), 3.54-3.47 (m, 2H), 3.35 (d, J=4.0 Hz, 4H), 3.19 (d, J=7.2 Hz, 1H), 3.17-3.05 (m, 4H), 2.93-2.84 (m, 1H), 2.79 (m, J=7.2, 12.3 Hz, 2H), 2.34-2.21 (m, 1H), 2.17-2.04 (m, 3H), 1.97-1.86 (m, 2H), 1.82 (d, J=11.2 Hz, 3H), 1.78-1.60 (m, 5H), 1.58-1.53 (m, 1H), 1.50 (s, 1H), 1.47 (s, 1H), 1.16-0.98 (m, 3H), 0.95-0.87 (m, 4H). F NMR (400 MHz, METHANOL-d4) δ=−77.061, −120.755.

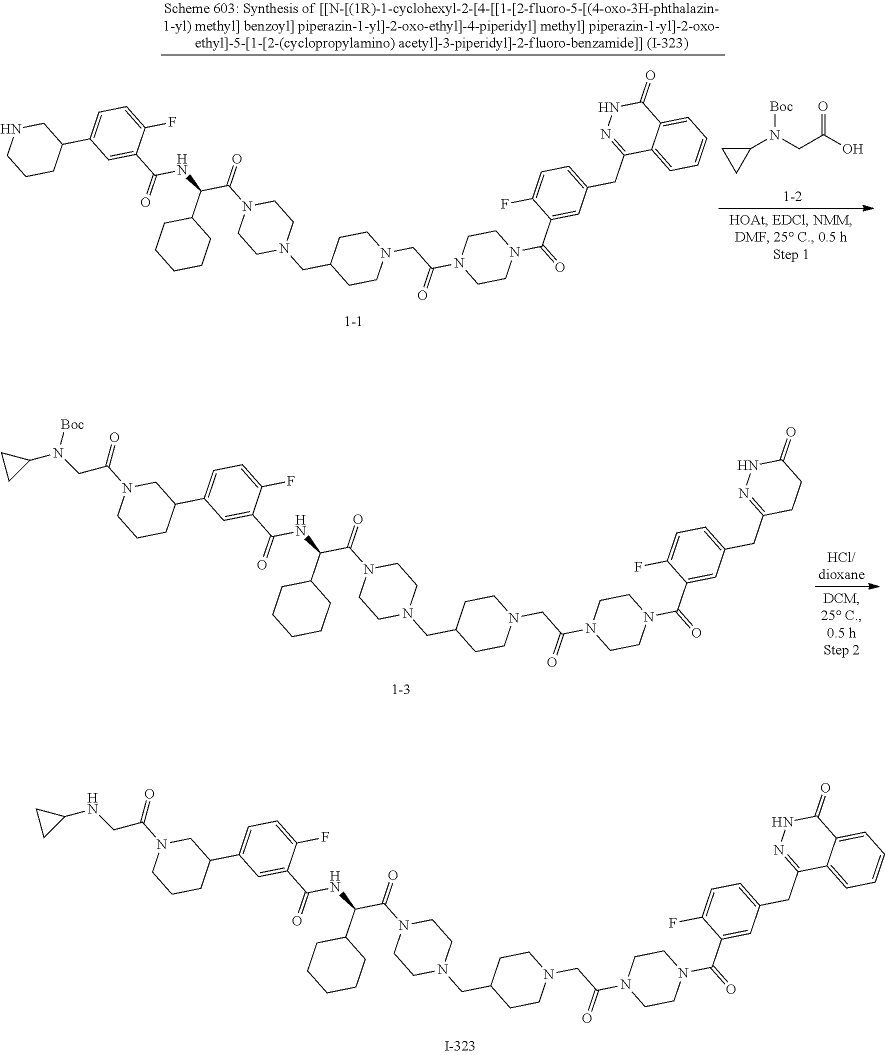

### Step 1: Synthesis of Intermediate 1-3

To a solution of Intermediate 1-1 (203 mg, 209.15 μmol, 1 eq, HCl salt) and Intermediate 1-2 (54.02 mg, 250.98 μmol, 1.2 eq) in DMF (2 mL) was added EDCI (120.28 mg, 627.46 μmol, 3 eq), NMM (105.78 mg, 1.05 mmol, 114.97 μL, 5 eq) and HOAt (28.47 mg, 209.15 μmol, 29.26 μL, 1 eq). The mixture was stirred at 25° C. for 0.5 hr. The reaction mixture was poured into H₂O (5 mL) and extracted with EA (5 mL*3). The combined organic layers were dried over Na₂SO₄, filtered and concentrated under reduced pressure to give a residue. The residue was purified by flash silica gel chromatography (ISCO; 12 g SepaFlash Silica Flash Column, Eluent of 0˜100% DCM/MeOH@36 mL/min, DCM/MeOH=10:1, Rf=0.3) and the eluent was concentrated under reduced pressure to give a residue. Intermediate 1-3 (159 mg, crude) was obtained as a white solid. LCMS (Method D): Rt=0.367 min, [M+H]+=1131.5.

### Step 2: Synthesis of I-323

To a solution of Intermediate 1-3 (159 mg, 140.54 μmol, 1 eq) in DCM (1 mL) was added HCl/dioxane (2 M, 0.3 mL, 4.27 eq) and the mixture was stirred at 25° C. for 0.5 hr. The reaction mixture was concentrated under reduced pressure to give a residue. The residue was purified by prep-HPLC (column: Phenomenex luna C18 150*25 mm*10 um; mobile phase: [water (HCl)-ACN]; gradient: 10%-40% B over 10 min) and concentrated under reduced pressure to remove ACN and lyophilized to give product. I-323 (61.19 mg, 57.31 μmol, 40.78% yield, 100% purity, HCl salt) was obtained as a white solid. LCMS (Method D): Rt=0.284 min, [M+H]+=1031.6. SFC: Rt=4.220 min, 5.670 min. ¹H NMR (400 MHz, METHANOL-d4) δ=8.37 (d, J=7.2 Hz, 1H), 8.00-7.93 (m, 1H), 7.92-7.79 (m, 2H), 7.72-7.58 (m, 1H), 7.57-7.45 (m, 2H), 7.38 (d, J=2.4 Hz, 1H), 7.27-7.12 (m, 2H), 4.89 (s, 2H), 4.73 (d, J=13.6 Hz, 1H), 4.66-4.43 (m, 3H), 4.41-4.35 (m, 3H), 4.32-4.26 (m, 1H), 4.26-4.15 (m, 1H), 3.84 (s, 2H), 3.80-3.66 (m, 6H), 3.56 (d, J=5.2 Hz, 3H), 3.47-3.33 (m, 5H), 3.25-3.15 (m, 4H), 3.11-2.96 (m, 1H), 2.92-2.72 (m, 3H), 2.46-2.33 (m, 1H), 2.23 (d, J=11.6 Hz, 2H), 2.12-1.96 (m, 2H), 1.91 (d, J=10.8 Hz, 3H), 1.84-1.52 (m, 8H), 1.36-1.26 (m, 2H), 1.20-1.11 (m, 2H), 1.00-0.86 (m, 4H). F NMR (400 MHz, CHLOROFORM-d) δ=−118.278, δ=−120.695.

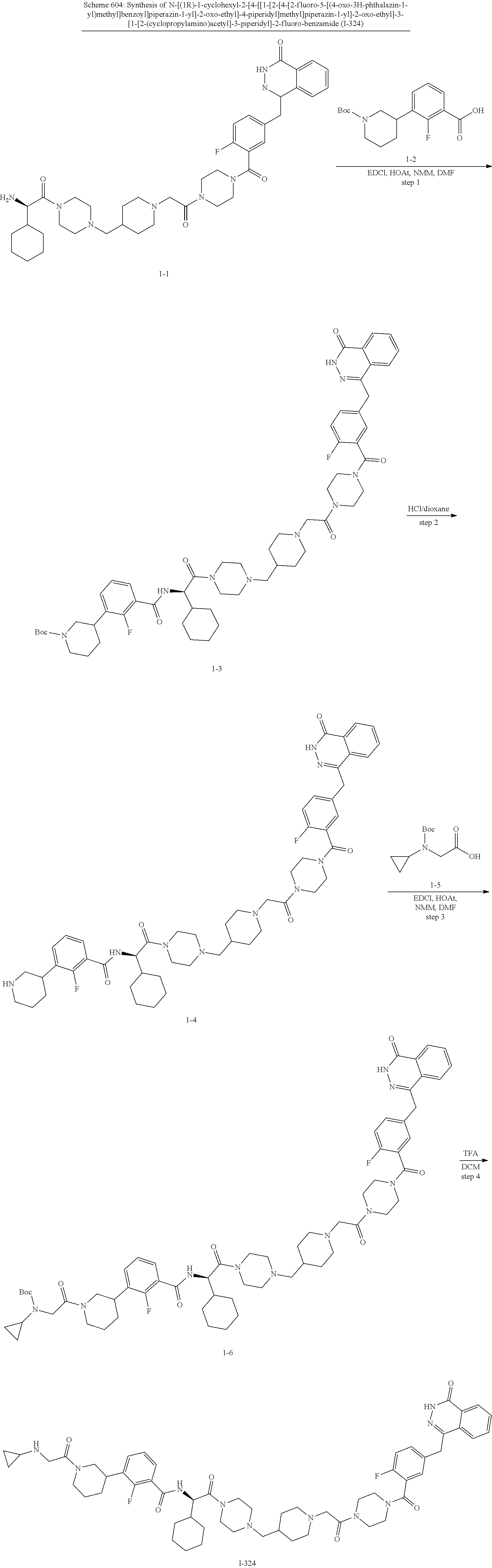

### Step 1: Synthesis of Intermediate 1-3

To a solution of Intermediate 1-2 (106.47 mg, 329.26 μmol, 1.2 eq) in DMF (2 mL) was added NMM (138.77 mg, 1.37 mmol, 150.83 μL, 5 eq), EDCI (263.00 mg, 1.37 mmol, 5 eq), HOAt (74.69 mg, 548.77 μmol, 76.77 μL, 2 eq), and Intermediate 1-1 (200 mg, 274.39 μmol, 1 eq). The mixture was stirred at 25° C. for 2 hr. The reaction was washed with water (2 mL) and extracted with EA (2 mL*3), the combined organic phase was dried by Na₂SO₄, and concentrated under reduced pressure to give a residue. The residue was purified by flash silica gel chromatography (ISCO; 12 g SepaFlash Silica Flash Column, Eluent of 0˜50% Ethyl acetate/Petroleum ether @45 mL/min) and the eluent was concentrated to give product. Intermediate 1-3 (380 mg, 273.56 μmol, 99.70% yield, 74.453% purity) was obtained as a yellow solid. LCMS (Method D): Retention time=0.395 min, [M+H]+=1034.6.

### Step 2: Synthesis of Intermediate 1-4

A solution of Intermediate 1-3 (380 mg, 271.38 μmol, 1 eq) in HCl/dioxane (2 M, 4.75 mL, 35.01 eq) was stirred at 25° C. for 1 hr. The mixture was concentrated under reduced pressure to give a residue. The crude product was used in the next step without further purification. Intermediate 1-4 (420 mg, crude, HCl salt) was obtained as a yellow solid. LCMS (Method D): Retention time=0.290 min, [M+H]+=934.6.

### Step 3: Synthesis of Intermediate 1-6

To a solution of Intermediate 1-5 (66.53 mg, 309.09 μmol, 1.5 eq) in DMF (2 mL) was added NMM (104.21 mg, 1.03 mmol, 113.27 μL, 5 eq), EDCI (197.51 mg, 1.03 mmol, 5 eq), HOAt (56.09 mg, 412.12 μmol, 57.65 μL, 2 eq) and Intermediate 1-4 (200 mg, 206.06 μmol, 1 eq, HCl salt). The mixture was stirred at 25° C. for 2 hr. The reaction was diluted with DCM, washed with water (2 mL) and extracted with DCM (2 mL*3), the combined organic phase was dried by Na₂SO₄, and concentrated under reduced pressure to give a residue. The residue was purified by reversed-phase chromatography (0.1% of FA) and the eluent was lyophilized to give product. Intermediate 1-6 (170 mg, 124.17 μmol, 60.26% yield, 82.636% purity) was obtained as a yellow solid. LCMS (Method D): Retention time=0.413 min, [M+H]+=1131.6.

### Step 4: Synthesis of I-324

To a solution of Intermediate 1-6 (170 mg, 150.26 μmol, 1 eq) in DCM (1.7 mL) was added TFA (1.30 g, 11.44 mmol, 0.85 mL, 76.15 eq) and the mixture was stirred at 25° C. for 1 hr. The mixture was concentrated under reduced pressure to give a residue. The crude was purified by prep-HPLC (column: CD04-Welch Utimate C18 150*25*7 um; mobile phase: [water (TFA)-ACN]; gradient: 10%-40% B over 10 min) and the eluent was concentrated to remove ACN and lyophilized to give product. I-324 (50 mg, 43.06 μmol, 28.66% yield, 98.638% purity, TFA salt) was obtained as a white solid. LCMS (Method D): Retention time=0.282, 0.286 min, [M+H]+=1031.5. SFC: Retention time=2.725 min, 3.404 min. ¹H NMR (400 MHz, METHANOL-d₄) δ=8.37 (d, J=7.2 Hz, 1H), 7.98-7.92 (m, 1H), 7.91-7.81 (m, 2H), 7.62-7.47 (m, 3H), 7.37 (t, J=6.8 Hz, 1H), 7.31-7.24 (m, 1H), 7.17 (t, J=8.8 Hz, 1H), 4.58 (d, J=10.4 Hz, 1H), 4.39 (s, 2H), 4.35-4.15 (m, 6H), 4.07-3.92 (m, 1H), 3.80 (d, J=15.6 Hz, 4H), 3.72 (s, 3H), 3.59-3.44 (m, 5H), 3.36 (d, J=7.2 Hz, 4H), 3.19 (d, J=5.6 Hz, 4H), 3.10-3.03 (m, 1H), 2.91-2.75 (m, 2H), 2.29 (d, J=6.0 Hz, 1H), 2.13 (d, J=13.2 Hz, 2H), 2.07-2.01 (m, 1H), 1.98-1.85 (m, 4H), 1.85 (s, 4H), 1.71 (s, 4H), 1.35-1.24 (m, 3H), 1.24-1.07 (m, 3H), 0.94-0.88 (m, 4H). ¹⁹F NMR (377 MHz, METHANOL-d₄) δ=−77.046, −120.732, −121.933.

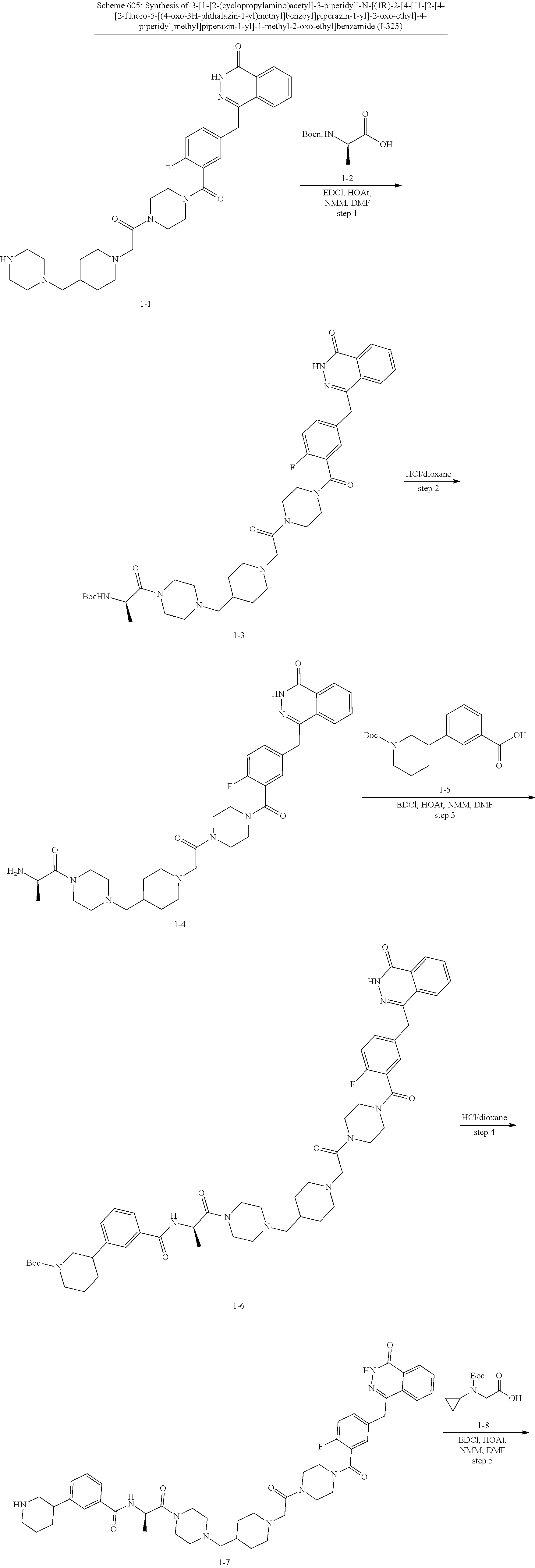

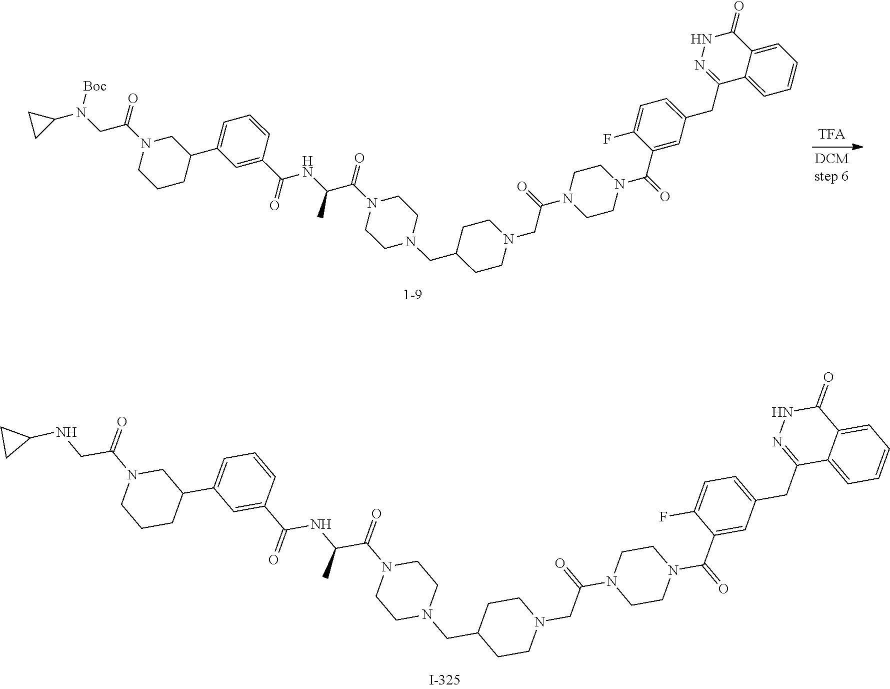 [SMILES: C[C@@H](NC(=O)c1cccc(C2CCCN(C(=O)CN(C(=O)OC(C)(C)C)C3CC3)C2)c1)C(=O)N1CCN(CC2CCN(CC(=O)N3CCN(C(=O)c4cc(Cc5n[nH]c(=O)c6ccccc56)ccc4F)CC3)CC2)CC1 ; C[C@@H](NC(=O)c1cccc(C2CCCN(C(=O)CNC3CC3)C2)c1)C(=O)N1CCN(CC2CCN(CC(=O)N3CCN(C(=O)c4cc(Cc5n[nH]c(=O)c6ccccc56)ccc4F)CC3)CC2)CC1]

### Step 1: Synthesis of Intermediate 1-3

To a solution of Intermediate 1-2 (64.17 mg, 339.15 μmol, 1 eq) in DMF (0.2 mL) was added NMM (171.52 mg, 1.70 mmol, 186.44 μL 5 eq), EDCI (325.08 mg, 1.70 mmol, 5 eq), HOAt (92.33 mg, 678.31 μmol, 94.89 μL, 2 eq), and Intermediate 1-2 (0.2 g, 339.15 μmol, 1 eq). The mixture was stirred at 25° C. for 2 hr. The reaction was washed with water (2 mL) and extracted with EA (2 mL*3), the combined organic phase was dried by Na₂SO₄, and concentrated under reduced pressure to give a residue. The residue was purified by flash silica gel chromatography (ISCO; 12 g SepaFlashSilica Flash Column, Eluent of 0-50% Ethyl acetate/Petroleum ether @45 mL/min) and the eluent was concentrated to give product. Intermediate 1-3 (305 mg, 330.36 μmol, 97.41% yield, 82.416% purity) was obtained as a yellow solid. LCMS (Method D): Retention time: 0.272 min, [M+H]+=761.4.

### Step 2: Synthesis of Intermediate 1-4

A solution of Intermediate 1-3 (305 mg, 400.84 μmol, 1 eq) in HCl/dioxane (2 M, 3 mL) was stirred at 25° C. for 1 hr. The mixture was concentrated under reduced pressure to give a residue. The crude was purified by prep-HPLC (column: CD01-Phenomenex luna C18 150*25*10 um; mobile phase: [water (FA)-ACN]; gradient: 0%-24% B over 14 min), and the eluent was concentrated to remove ACN and lyophilized to give product. Intermediate 1-4 (255 mg, 365.73 μmol, 91.24% yield, 100% purity, HCl salt) was obtained as a colorless oil. LCMS (Method D): Retention time: 0.217 min, [M+H]+=661.4. SFC: Retention time: 2.502 min.

### Step 3: Synthesis of Intermediate 1-6

To a solution of Intermediate 1-5 (131.39 mg, 430.27 μmol, 1.5 eq) in DMF (2 mL) was added NMM (145.07 mg, 1.43 mmol, 157.68 μL, 5 eq), EDCI (274.94 mg, 1.43 mmol, 5 eq), HOAt (78.09 mg, 573.69 μmol, 80.25 μL, 2 eq), and Intermediate 1-4 (200 mg, 286.85 μmol, 1 eq, HCl salt). The mixture was stirred at 25° C. for 2 hr. The reaction was washed with water (2 mL) and extracted with EA (2 mL*3), the combined organic phase was dried by Na₂SO₄, and concentrated under reduced pressure to give a residue. The residue was purified by reversed-phase chromatography (0.1% of FA) and the eluent was lyophilized to give product. Intermediate 1-6 (300 mg, 271.65 μmol, 94.70% yield, 85.854% purity) was obtained as a yellow oil. LCMS (Method D): Retention time: 0.390 min, [M+H]+=948.5.

### Step 4: Synthesis of Intermediate 1-7

A solution of Intermediate 1-6 (300 mg, 316.41 μmol, 1 eq) in HCl/dioxane (2 M, 3 mL) was stirred at 25° C. for 1 hr. The mixture was concentrated under reduced pressure to give a residue. The crude product was used in the next step without further purification. Intermediate 1-7 (300 mg, crude, HCl salt) was obtained as a yellow solid. LCMS (Method D): Retention time: 0.237 min, [M+H]+=848.7.

### Step 5: Synthesis of Intermediate 1-9

To a solution of Intermediate 1-8 (36.50 mg, 169.59 μmol, 1.5 eq) in DMF (1 mL) was added NMM (57.18 mg, 565.31 μmol, 62.15 μL, 5 eq), EDCI (108.37 mg, 565.31 μmol, 5 eq), HOAt (30.78 mg, 226.12 μmol, 31.63 μL, 2 eq), and Intermediate 1-7 (100 mg, 113.06 μmol, 1 eq, HCl salt). The mixture was stirred at 25° C. for 2 hr. The reaction was washed with water (1 mL) and extracted with DCM (1 mL*3), the combined organic phase was dried by Na₂SO₄, and concentrated under reduced pressure to give a residue. The residue was purified by flash silica gel chromatography (ISCO; 4 g SepaFlash Silica Flash Column, Eluent of 0˜50% Methanol/Dichloromethane @20 mL/min) and the eluent was concentrated to give product. Intermediate 1-9 (65 mg, 60.16 μmol, 53.21% yield, 96.749% purity) was obtained as a white solid. LCMS (Method D): Retention time: 0.335 min, [M+H]+=1045.4.

### Step 6: Synthesis of I-325

To a solution of Intermediate 1-9 (55 mg, 52.62 μmol, 1 eq) in DCM (0.5 mL) was added TFA (383.75 mg, 3.37 mmol, 0.25 mL, 63.96 eq) and the mixture was stirred at 25° C. for 1 hr. The mixture was concentrated under reduced pressure to give a residue. The crude was purified by prep-HPLC (column: CD04-Welch Utimate C18 150*25*7 um; mobile phase: [water (TFA)-ACN]; gradient: 1%-31% B over 10 min), and the eluent was concentrated to remove ACN and lyophilized to give product. I-325 (22 mg, 20.72 μmol, 39.37% yield, 99.731% purity, TFA salt) was obtained as a yellow solid. LCMS (Method D): Retention time: 0.258 min, [M+H]+=945.5. SFC: Retention time: 2.381 min, 2.655 min. ¹H NMR (400 MHz, METHANOL-d₄) δ=8.39 (d, J=7.2 Hz, 1H), 8.01-7.94 (m, 1H), 7.94-7.81 (m, 3H), 7.80-7.74 (m, 1H), 7.58-7.49 (m, 2H), 7.49-7.44 (m, 1H), 7.42-7.36 (m, 1H), 7.19 (t, J=9.0 Hz, 1H), 5.09-5.01 (m, 1H), 4.63-4.54 (m, 1H), 4.41 (s, 2H), 4.35-4.28 (m, 2H), 4.28-4.13 (m, 3H), 4.04-3.93 (m, 1H), 3.82 (d, J=15.6 Hz, 3H), 3.74 (s, 3H), 3.62-3.51 (m, 3H), 3.49-3.42 (m, 2H), 3.43-3.35 (m, 4H), 3.31-3.08 (m, 6H), 2.97-2.86 (m, 1H), 2.85-2.75 (m, 2H), 2.38-2.25 (m, 1H), 2.19-2.06 (m, 3H), 1.96-1.85 (m, 2H), 1.82-1.61 (m, 3H), 1.54-1.46 (m, 3H), 1.45-1.28 (m, 1H), 0.97-0.89 (m, 4H). ¹⁹F NMR (377 MHz, METHANOL-d₄) δ=−77.031, −120.740.

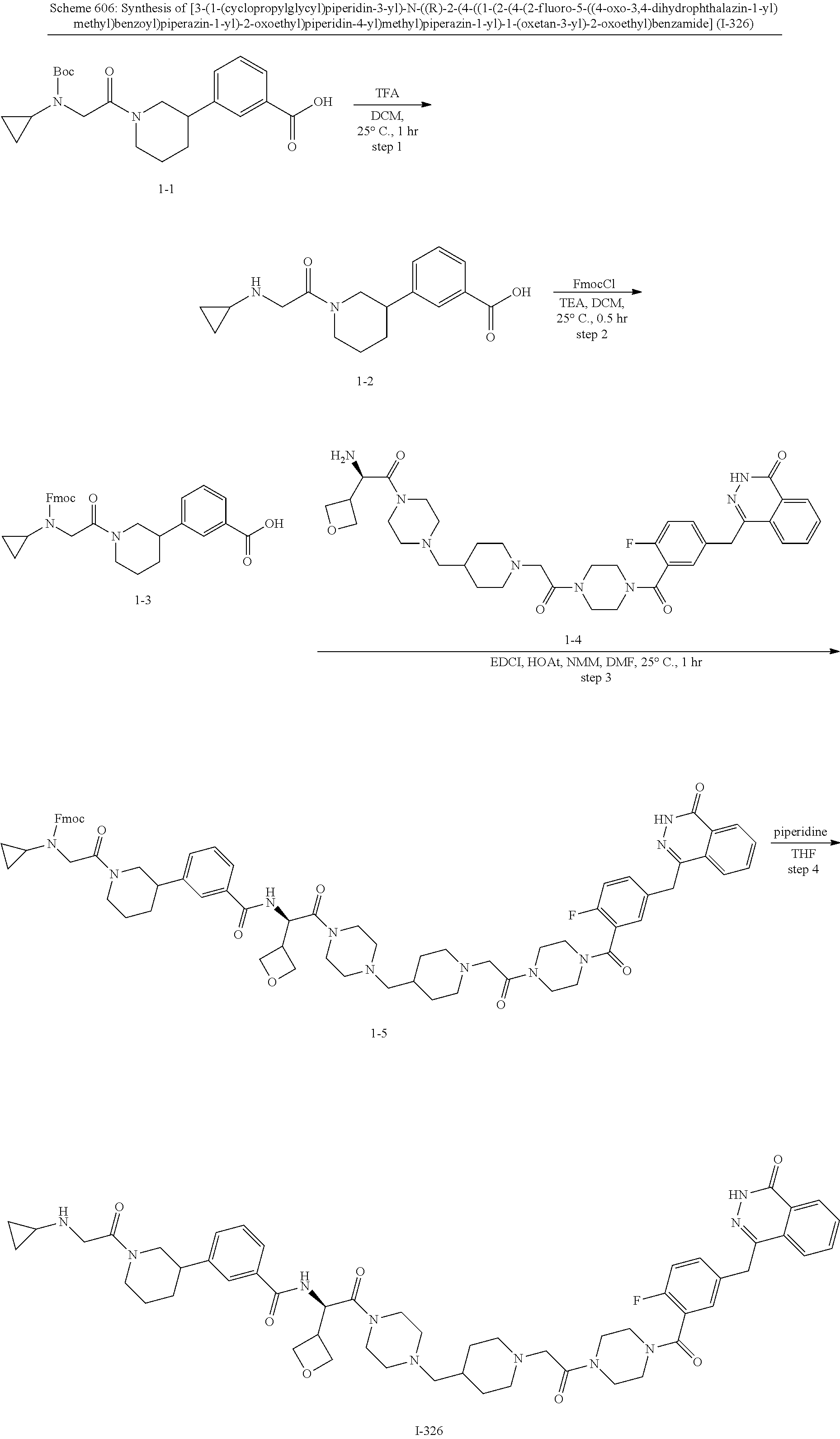

### Step 1: Synthesis of Intermediate 1-2.

To a solution of Intermediate 1-1 (500 mg, 1.24 mmol, 1 eq) in DCM (5 mL) was added TFA (1 mL). The mixture was stirred at 25° C. for 1 hr. The reaction mixture was concentrated under reduced pressure to remove DCM. The residue was washed with PE (30 mL). The crude product was used in the next step without further purification. Intermediate 1-2 (520 mg, crude, TFA salt) was obtained as a yellow oil. LCMS (Method D): Retention time: 0.238 min, [M+H]+=303.1.

### Step 2: Synthesis of Intermediate 1-3.

To a solution of Intermediate 1-2 (520 mg, 1.25 mmol, 1 eq, TFA salt) in DCM (5 mL) was added TEA (379.10 mg, 3.75 mmol, 521.46 μL, 3 eq) and Fmoc-Cl (387.68 mg, 1.50 mmol, 1.2 eq). The mixture was stirred at 25° C. for 0.5 hr. The reaction mixture was diluted with H₂O (10 mL) and extracted with EA (10 mL*3). The combined organic layers were dried over Na₂SO₄, filtered and concentrated under reduced pressure to give a residue. The residue was purified by flash silica gel chromatography (ISCO; 20 g SepaFlash Silica Flash Column, Eluent of 0˜100% Ethyl acetate/Petroleum ether gradient @60 mL/min), and the eluent was concentrated under reduced pressure to give a residue. Intermediate 1-3 (200 mg, 294.13 μmol, 23.55% yield, 77.150% purity) was obtained as a yellow oil. LCMS (Method D): Retention time: 0.468 min, [M+H]+=525.2.

### Step 3: Synthesis of Intermediate 1-5.

To a solution of Intermediate 1-3 (70 mg, 133.43 μmol, 1 eq) in DMF (1 mL) was added HOAt (36.32 mg, 266.87 μmol, 37.33 μL, 2 eq), EDCI (127.90 mg, 667.17 μmol, 5 eq) and NMM (134.96 mg, 1.33 mmol, 146.70 μL, 10 eq). Then Intermediate 1-4 (93.78 mg, 133.43 μmol, 1 eq) was added. The mixture was stirred at 25° C. for 1 hr. The reaction mixture was diluted with H₂O (5 mL) and extracted with EA (5 mL*3). The combined organic layers were washed with brine (5 mL*3), dried over Na₂SO₄, filtered and concentrated under reduced pressure to give a residue. The residue was purified by prep-HPLC (FA condition), the eluent was concentrated and lyophilized to give the desired product. Intermediate 1-5 (60 mg, 44.65 μmol, 33.47% yield, 93.433% purity, FA salt) was obtained as a white solid. LCMS (Method D): Retention time: 0.387 min, [M+H]+=1210.1. ¹H NMR (400 MHz, METHANOL-d4) δ=8.36 (d, J=7.6 Hz, 1H), 7.97-7.91 (m, 1H), 7.89-7.67 (m, 7H), 7.58-7.46 (m, 3H), 7.44-7.26 (m, 6H), 7.21-7.13 (m, 1H), 4.83-4.79 (m, 2H), 4.61-4.49 (m, 4H), 4.44-4.35 (m, 3H), 4.29-4.14 (m, 2H), 3.90-3.42 (m, 16H), 3.18-2.97 (m, 3H), 2.78-2.59 (m, 3H), 2.52-2.27 (m, 6H), 2.23-1.98 (m, 4H), 1.90-1.76 (m, 4H), 1.70-1.48 (m, 2H), 1.38-1.22 (m, 2H), 0.72-0.42 (m, 4H). ¹⁹F NMR (376 MHz, METHANOL-d4) δ=−120.72.

### Step 4: Synthesis of I-326.

To a solution of Intermediate 1-5 (60 mg, 47.79 μmol, 1 eq, FA salt) in THF (0.8 mL) was added piperidine (0.1 mL). The mixture was stirred at 25° C. for 1 hr. The reaction mixture was diluted with H₂O (5 mL) and extracted with EA (5 mL*3). The combined organic layers were concentrated under reduced pressure to give a residue. The residue was purified by prep-HPLC (column: CD02-Waters Xbidge BEH C18 150*25*10 um; mobile phase: [water (NH₃H₂O)-ACN]; gradient: 20%-50% B over 10 min), and the eluent was concentrated and lyophilized to give the desired product. I-326 (16.48 mg, 16.69 μmol, 34.93% yield, 100% purity) was obtained as a white solid. LCMS (Method F): Retention time: 0.552 min, [M+H]+=987.7. SFC: Retention time: 1.790, 1.949 min. ¹H NMR (400 MHz, METHANOL-d4) δ=8.40-8.33 (m, 1H), 7.98-7.93 (m, 1H), 7.90-7.83 (m, 2H), 7.82-7.78 (m, 1H), 7.76-7.71 (m, 1H), 7.54-7.43 (m, 3H), 7.37 (s, 1H), 7.21-7.10 (m, 1H), 5.52-5.43 (m, 1H), 4.83-4.76 (m, 3H), 4.71-4.64 (m, 1H), 4.58 (d, J=10.4 Hz, 1H), 4.45-4.35 (m, 3H), 3.96-3.87 (m, 1H), 3.81-3.71 (m, 4H), 3.66 (s, 3H), 3.61-3.55 (m, 4H), 3.52 (s, 2H), 3.25-3.10 (m, 3H), 2.95-2.70 (m, 5H), 2.49-2.32 (m, 4H), 2.27-2.16 (m, 3H), 2.05 (d, J=10.0 Hz, 3H), 1.92-1.83 (m, 2H), 1.77-1.70 (m, 2H), 1.63 (s, 1H), 1.57-1.47 (m, 1H), 1.29-1.14 (m, 2H), 0.51-0.43 (m, 2H), 0.42-0.34 (m, 2H). ¹⁹F NMR (376 MHz, METHANOL-d4) δ=−120.70.

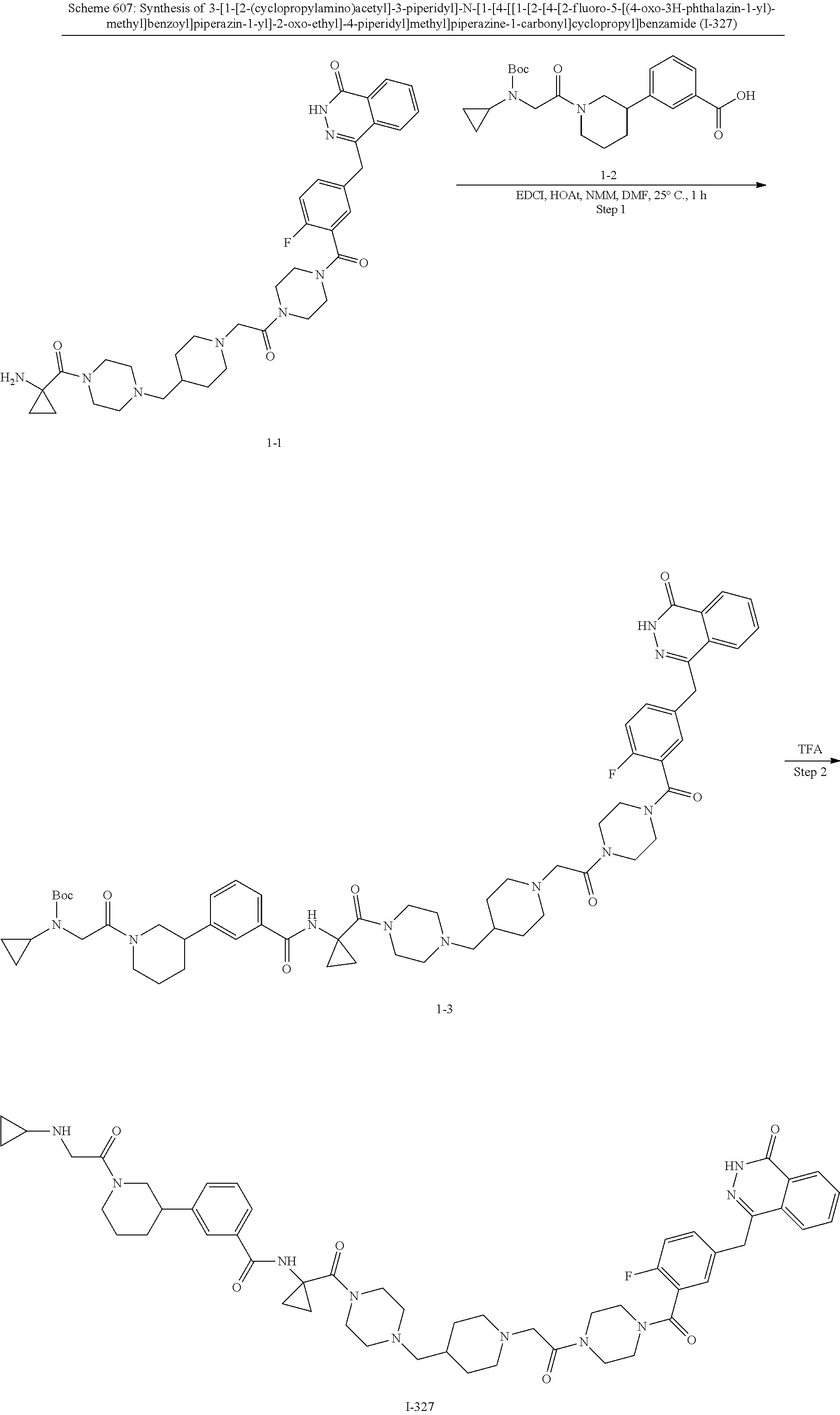 [SMILES: NC1(C(=O)N2CCN(CC3CCN(CC(=O)N4CCN(C(=O)c5cc(Cc6n[nH]c(=O)c7ccccc67)ccc5F)CC4)CC3)CC2)CC1 ; CC(C)(C)OC(=O)N(CC(=O)N1CCCC(c2cccc(C(=O)O)c2)C1)C1CC1 ; CC(C)(C)OC(=O)N(CC(=O)N1CCCC(c2cccc(C(=O)NC3(C(=O)N4CCN(CC5CCN(CC(=O)N6CCN(C(=O)c7cc(Cc8n[nH]c(=O)c9ccccc89)ccc7F)CC6)CC5)CC4)CC3)c2)C1)C1CC1 ; O=C(NC1(C(=O)N2CCN(CC3CCN(CC(=O)N4CCN(C(=O)c5cc(Cc6n[nH]c(=O)c7ccccc67)ccc5F)CC4)CC3)CC2)CC1)c1cccc(C2CCCN(C(=O)CNC3CC3)C2)c1]

### Step 1: Synthesis of Intermediate 1-3

To solution of intermediate 1-1 (80 mg, 198.77 μmol, 1 eq) in DCM (1 mL) was added EDCI (114.31 mg, 596.30 μmol, 3 eq), NMM (100.52 mg, 993.83 μmol, 109.26 μL, 5 eq) and HOAt (27.05 mg, 198.77 μmol, 27.81 μL, 1 eq). The mixture was stirred at 25° C. for 0.5 hr. Then intermediate 1-2 (156.39 mg, 198.77 μmol, 1 eq, TFA salt) was added into the mixture and it was stirred at 25° C. for 0.5 hr. The reaction mixture was diluted with water (9 mL) and extracted with ethyl acetate (3 mL×3). The combined organic layers were washed with brine (3 mL×3) and dried over sodium sulfate, filtered and concentrated under reduced pressure to give a residue. The residue was purified by prep-HPLC (column: CD06-Waters Xbidge C18 150*40*10 um; mobile phase: [water (NH₄HCO₃)-ACN]; gradient: 26%-566% B over 10 min). The eluent was concentrated under reduced pressure to remove ACN and then lyophilized to give intermediate 1-3 (60 mg, 55.82 μmol, 28.08% yield, 98.36% purity) as a white solid. LCMS (Method D): Rt: 0.347 min, (M+H)=1057.4. SFC: Rt: 2.740, 2.880 min. ¹H NMR (400 MHz, CHLOROFORM-d) δ=10.65-10.02 (m, 1H), 8.52-8.39 (m, 1H), 7.81-7.57 (m, 5H), 7.43-7.30 (m, 4H), 7.22-7.03 (m, 2H), 4.76-4.58 (m, 1H), 4.29 (s, 2H), 4.16-3.96 (m, 2H), 3.91-3.69 (m, 8H), 3.64-3.54 (m, 2H), 3.50 (s, 2H), 3.38-3.21 (m, 2H), 3.21-3.13 (m, 2H), 2.90-2.74 (m, 4H), 2.40 (s, 4H), 2.16 (d, J=6.8 Hz, 2H), 2.09-2.04 (m, 2H), 1.90-1.70 (m, 6H), 1.47 (s, 9H), 1.39-1.12 (m, 8H), 0.75-0.57 (m, 4H). ¹⁹F NMR (376 MHz, METHAN OL-d4) δ=−117.544.

### Step 2: Synthesis of I-327

To a solution of intermediate 1-3 (50 mg, 47.29 μmol, 1 eq) in DCM (0.5 mL) was added TFA (153.50 mg, 1.35 mmol, 100 μL, 28.47 eq). The mixture was stirred at 25° C. for 1 hr. The mixture was adjusted to pH=7-8 with an aqueous solution of sodium bicarbonate. The reaction mixture was diluted with MeOH (2 mL). The crude product was purified by reversed-phase (0.1% NH₃·H₂O). The eluent was concentrated under reduced pressure to remove ACN and then lyophilized to give I-327 (15.73 mg, 16.26 μmol, 34.37% yield, 98.91% purity) as a white solid. LCMS (Method D): Rt: 0.257 min, (M+H)=957.5. SFC: Rt: 0.958, 1.268 min. ¹H NMR (400 MHz, METHANOL-d4) δ=8.36 (d, J=8.0 Hz, 1H), 7.98-7.92 (m, 1H), 7.90-7.78 (m, 2H), 7.73 (d, J=4.8 Hz, 1H), 7.67 (d, J=7.2 Hz, 1H), 7.54-7.33 (m, 4H), 7.17-7.13 (m, 1H), 4.58 (d, J=9.2 Hz, 1H), 4.38 (s, 2H), 3.91-3.89 (m, 1H), 3.82-3.43 (m, 12H), 3.35-3.32 (m, 1H), 3.30-3.27 (m, 1H), 3.24 (s, 1H), 3.22-3.08 (m, 2H), 2.94-2.82 (m, 2H), 2.81-2.55 (m, 2H), 2.36 (d, J=3.2 Hz, 4H), 2.27-2.19 (m, 1H), 2.15-2.14 (m, 2H), 2.05-2.03 (m, 3H), 1.92-1.79 (m, 2H), 1.72-1.70 (m, 2H), 1.66-1.45 (m, 2H), 1.39 (s, 2H), 1.33-1.16 (m, 2H), 1.15-1.09 (m, 2H), 0.59-0.29 (m, 4H). ¹⁹F NMR (376 MHz, METHANOL-d4) δ=−120.779.

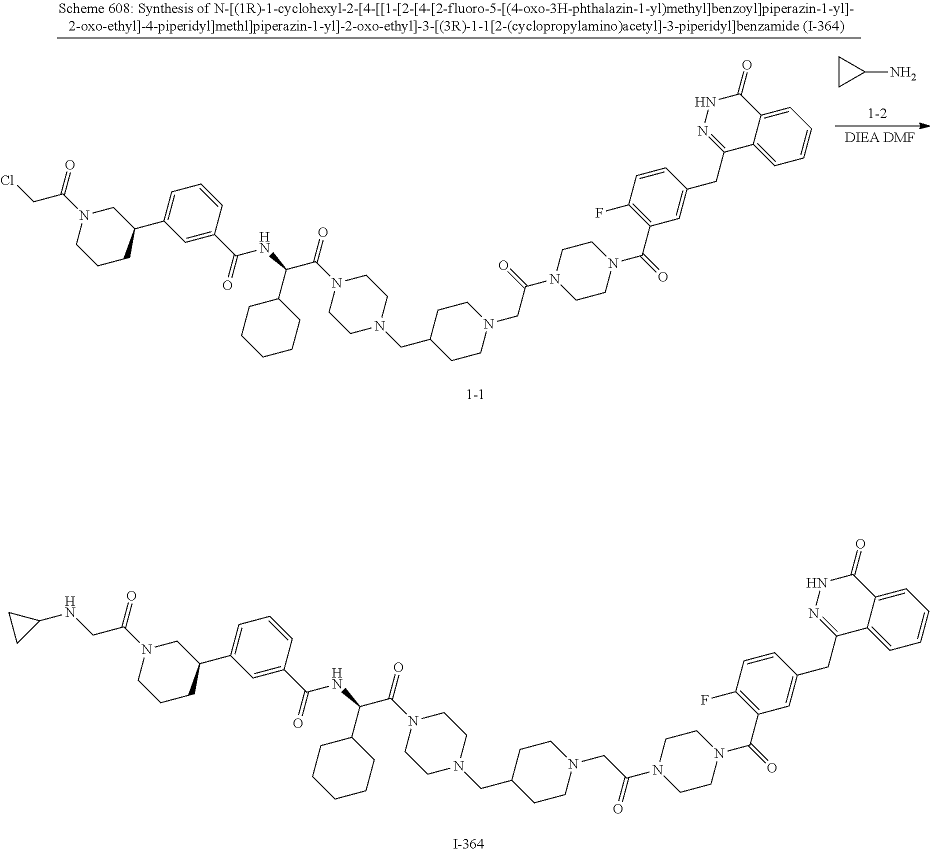

To a solution of Intermediate 1-1 (700 mg, 705.21 μmol, 1 eq) in DMF (7 mL) was added DIEA (273.43 mg, 2.12 mmol, 368.50 μL, 3 eq) and Intermediate 1-2 (60.39 mg, 1.06 mmol, 73.29 μL, 1.5 eq). The mixture was stirred at 40° C. for 12 hr. The mixture was poured into H₂O (30 mL) and stirred for 0.1 hr, and then filtered to collect the solid (500 mg, 493.46 μmol, 69.97% yield). The solid (400 mg) was purified by prep-HPLC (FA condition). The eluent was concentrated under reduced pressure to remove ACN and lyophilized to give product (244.88 mg, 231.18 μmol, 58.56% yield, FA salt) as a white solid. The white solid (100 mg) was further purified by Prep-HPLC (column: Phenomenex Luna C18 150*25 mm*10 um; mobile phase: [water (FA)-ACN]; gradient: 5%-35% B over 9 min) and concentrated to remove MeCN and then lyophilized to afford I-364 (90 mg, 86.16 μmol, 87.30% yield, 97% purity) as a white solid. LCMS (Method E): Rt=0.395 min, M+H=1013.8. SFC: Rt=3.707 min ¹H NMR (400 MHz, METHANOL-d₄) δ=8.55 (s, 1H), 8.37 (d, J=7.6 Hz, 1H), 7.98-7.91 (m, 1H), 7.90-7.75 (m, 3H), 7.74-7.68 (m, 1H), 7.52-7.35 (m, 4H), 7.18-7.14 (m, 1H), 4.95-4.89 (m, 1H), 4.61-4.52 (m, 1H), 4.38 (s, 2H), 3.92-3.49 (m, 13H), 3.37-3.32 (m, 1H), 3.25-3.10 (m, 2H), 2.98-2.68 (m, 4H), 2.54-2.33 (m, 4H), 2.28-2.01 (m, 6H), 1.97-1.50 (m, 13H), 1.44-0.97 (m, 8H), 0.54-0.35 (m, 4H).

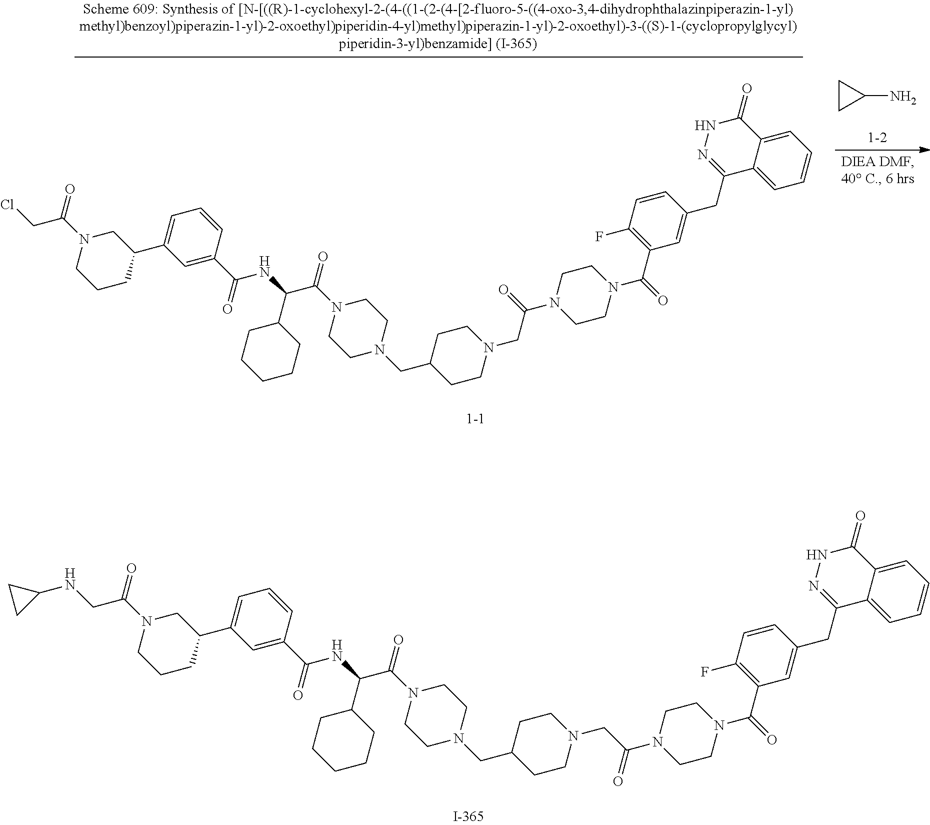

To a solution of Intermediate 1-1 (700 mg, 705.21 μmol, 1 eq) and Intermediate 1-2 (40.26 mg, 705.21 μmol, 48.86 μL, 1 eq) in DMF (7 mL) was added DIEA (273.43 mg, 2.12 mmol, 368.50 μL, 3 eq). The mixture was stirred at 40° C. for 6 hrs. The reaction mixture was diluted with H₂O (10 mL) and extracted with EA (10 mL*3). The combined organic layers were washed with brine (10 mL*3), dried over Na₂SO₄, filtered and concentrated under reduced pressure to give a residue. The residue was purified by prep-HPLC (FA condition), and the eluent was concentrated and lyophilized to give the desired product. I-365 (151.95 mg, 134.74 μmol, 19.11% yield, 93.933% purity, FA salt) was obtained as a white solid. LCMS (Method D): Retention time: 0.293 min, [M+H]⁺=1013.4. ¹H NMR (400 MHz, METHANOL-d₄) δ=8.37 (d, J=7.6 Hz, 1H), 7.99-7.91 (m, 1H), 7.91-7.81 (m, 2H), 7.80-7.70 (m, 2H), 7.56-7.41 (m, 3H), 7.41-7.33 (m, 1H), 7.22-7.11 (m, 1H), 4.92 (s, 1H), 4.61-4.51 (m, 1H), 4.39 (s, 2H), 4.10-3.88 (m, 4H), 3.82 (d, J=3.2 Hz, 3H), 3.74 (d, J=16.8 Hz, 4H), 3.62-3.52 (m, 3H), 3.45-3.34 (m, 4H), 3.24-3.14 (m, 1H), 2.95-2.65 (m, 5H), 2.63-2.39 (m, 5H), 2.30 (s, 2H), 2.07 (d, J=12.0 Hz, 1H), 2.01-1.74 (m, 9H), 1.70 (d, J=12.0 Hz, 3H), 1.53-1.41 (m, 2H), 1.41-1.34 (m, 1H), 1.33-1.20 (m, 3H), 1.17-1.04 (m, 2H), 0.78-0.71 (m, 3H). ¹⁹F NMR (376 MHz, CHLOROFORM-d) δ=−120.68.

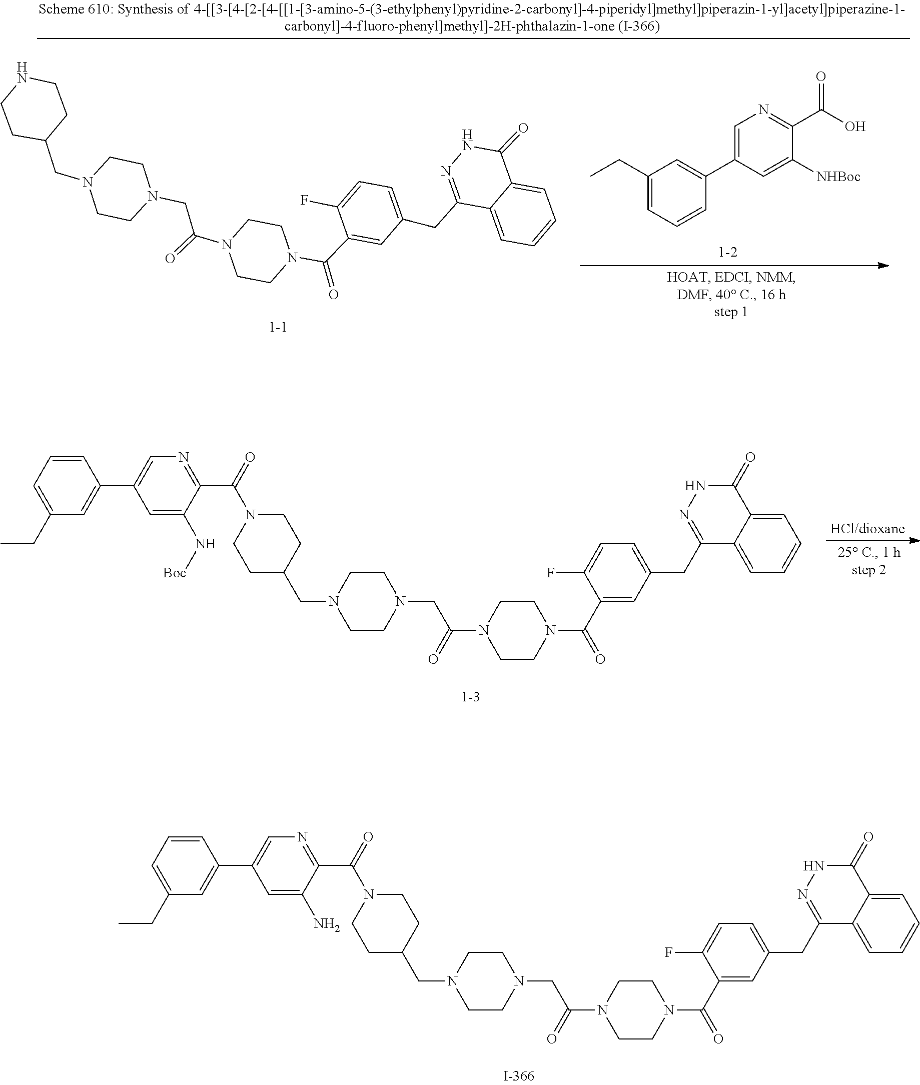 [SMILES: O=C(CN1CCN(CC2CCNCC2)CC1)N1CCN(C(=O)c2cc(Cc3n[nH]c(=O)c4ccccc34)ccc2F)CC1 ; CCc1cccc(-c2cnc(C(=O)O)c(NC(=O)OC(C)(C)C)c2)c1 ; CCc1cccc(-c2cnc(C(=O)N3CCC(CN4CCN(CC(=O)N5CCN(C(=O)c6cc(Cc7n[nH]c(=O)c8ccccc78)ccc6F)CC5)CC4)CC3)c(NC(=O)OC(C)(C)C)c2)c1 ; CCc1cccc(-c2cnc(C(=O)N3CCC(CN4CCN(CC(=O)N5CCN(C(=O)c6cc(Cc7n[nH]c(=O)c8ccccc78)ccc6F)CC5)CC4)CC3)c(N)c2)c1]

### Step 1: Synthesis of Intermediate 1-3

To a mixture of intermediate 1-2 (1.5 g, 4.38 mmol, 1 eq) and intermediate 1-1 (2.74 g, 4.38 mmol, 1 eq, HCl salt) in DMF (15 mL) was added EDCI (2.52 g, 13.14 mmol, 3 eq), HOAT (596.30 mg, 4.38 mmol, 612.85 μL, 1 eq) and NMM (2.22 g, 21.90 mmol, 2.41 mL, 5 eq). The mixture was stirred at 40° C. for 16 hrs. The reaction was poured into H₂O (15 mL) and extracted with DCM (20 mL*3). The combined organic layers were dried over Na₂SO₄, filtered and concentrated under reduced pressure to give the crude product. The residue was purified by flash silica gel chromatography (ISCO; 40 g SepaFlash Silica Flash Column, Eluent of 0˜70% Ethyl acetate/Methanol gradient @40 mL/min) and concentrated under reduced pressure to give a residue. Intermediate 1-3 (2 g, 2.14 mmol, 48.83% yield, 97.778% purity) was obtained as a yellow solid. LCMS (Method D): Rt=0.402 min, [M+H]⁺=914.5

### Step 2: Synthesis of I-366

A solution of intermediate 1-3 (2 g, 2.19 mmol, 1 eq) was added HCl/dioxane (2 M, 20 mL, 18.28 eq). The mixture was stirred at 25° C. for 1 hr. The reaction mixture was concentrated under reduced pressure to give a residue. The crude product was purified by reversed-phase HPLC (0.1% HCl condition) followed by lyophilization to give a residue. I-366 (335.2 mg, 389.33 μmol, 17.79% yield, 98.775% purity, HCl salt) was obtained as a yellow solid. LCMS (Method D): Rt=0.334 min, [M+H]⁺=814.4. ¹H NMR (400 MHz, METHANOL-d₄) δ=8.38-8.35 (m, 2H), 8.05 (s, 1H), 7.98-7.94 (m, 1H), 7.92-7.87 (m, 1H), 7.86-7.81 (m, 1H), 7.59 (s, 1H), 7.56-7.50 (m, 2H), 7.49-7.45 (m, 1H), 7.41-7.36 (m, 2H), 7.17 (t, J=8.8 Hz, 1H), 4.56 (d, J=16.8 Hz, 2H), 4.39 (s, 2H), 3.91 (s, 6H), 3.88-3.70 (m, 7H), 3.56 (s, 2H), 3.42 (s, 2H), 3.35 (d, J=1.6 Hz, 2H), 3.28-3.16 (m, 2H), 2.79-2.73 (m, 2H), 2.35 (s, 1H), 2.08 (d, J=12.4 Hz, 2H), 1.51-1.41 (m, 2H), 1.30 (t, J=7.6 Hz, 4H). ¹⁹F NMR (376 MHz, METHANOL-d₄) δ=−120.742.

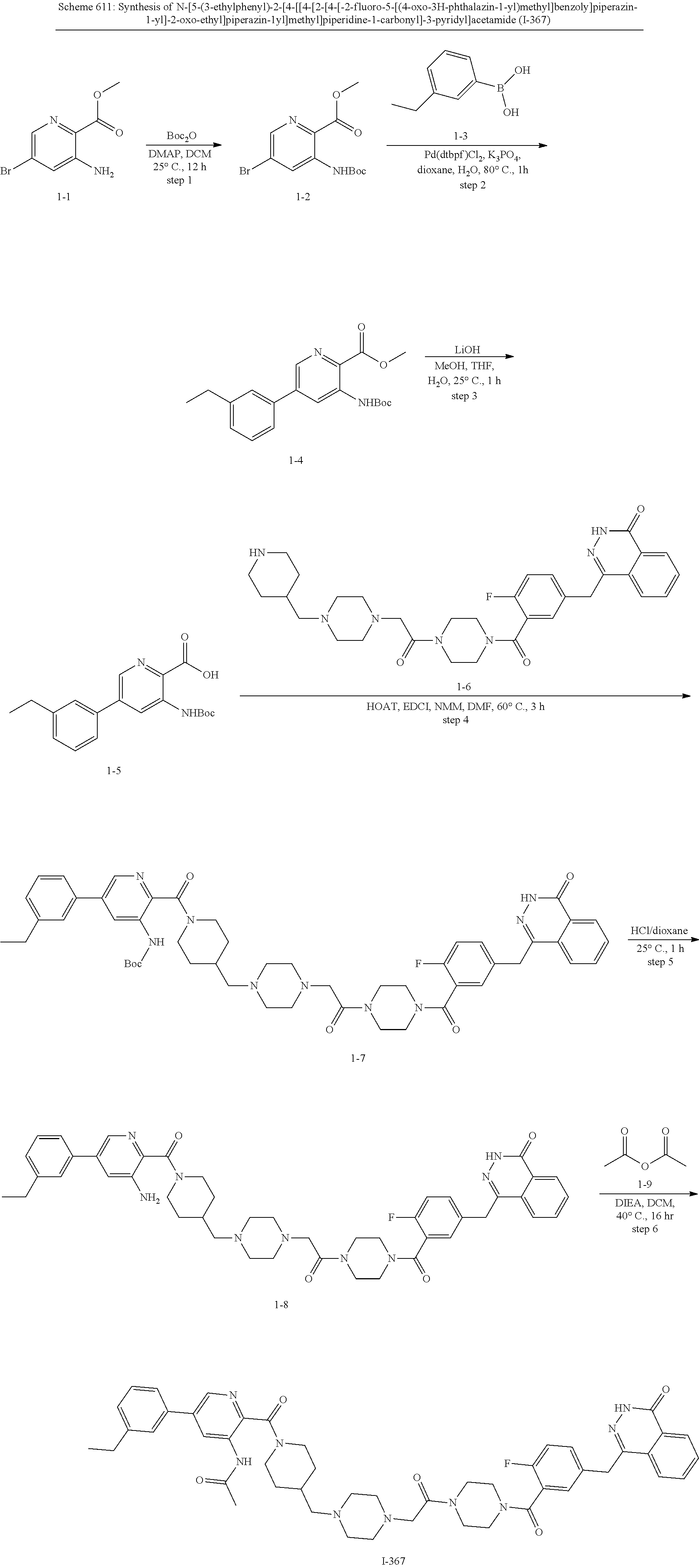

### Step 1: Synthesis of Intermediate 1-2

To a solution of intermediate 1-1 (20 g, 86.56 mmol, 1 eq) in DCM (200 mL) was added (Boc)₂O (22.67 g, 103.88 mmol, 23.86 mL, 1.2 eq) and DMAP (1.06 g, 8.66 mmol, 0.1 eq). The mixture was stirred at 25° C. for 12 h. The residue was poured into water (100 mL). The aqueous phase was extracted with EA (50 mL*3). The combined organic phase was washed with brine (100 mL*3), dried with anhydrous sodium sulfate, filtered, and concentrated in vacuo to give a residue. The residue was purified by flash silica gel chromatography (ISCO; 40 g SepaFlash Silica Flash Column, Eluent of 0˜20% Ethyl acetate/Petroleum ether gradient @100 mL/min) and concentrated under vacuum. Intermediate 1-2 (18 g, 54.35 mmol, 62.79% yield) was obtained as a brown solid. ¹H NMR (400 MHz, DMSO-d₆) δ=9.98 (s, 1H), 8.69 (d, J=2.4 Hz, 1H), 8.42 (d, J=2.4 Hz, 1H), 3.86 (s, 3H), 1.48 (s, 9H).

### Step 2: Synthesis of Intermediate 1-4

To a solution of intermediate 1-2 (10 g, 30.20 mmol, 1 eq) and intermediate 1-3 (5.43 g, 36.24 mmol, 1.2 eq) in dioxane (100 mL) and H₂O (20 mL) was added Pd(dtbpf)Cl₂ (1.97 g, 3.02 mmol, 0.1 eq) and K₃PO₄ (19.23 g, 90.59 mmol, 3 eq), and it was degassed and purged with N₂ 3 times. The mixture was stirred at 80° C. for 1 hr under N₂ atmosphere. The reaction mixture was washed with H₂O (500 mL) at 25° C., and the mixture was extracted with EA (500 ml*3), and the combined organic phase was dried with anhydrous sodium sulfate, filtered and the filtrate was concentrated to give the crude product. The residue was purified by flash silica gel chromatography (ISCO; 120 g SepaFlash Silica Flash Column, Eluent of 0˜35% Ethyl acetate/Petroleum ether gradient @100 mL/min) to give a residue. Intermediate 1-4 (11.6 g, 31.57 mmol, 93.77% yield, 97% purity) was obtained as a brown oil. LCMS (Method D): Rt=0.470 min, [M+H]⁺=357.1. ¹H NMR (400 MHz, METHANOL-d₄) δ=8.98 (d, J=2.0 Hz, 1H), 8.46-8.38 (m, 1H), 7.50-7.43 (m, 2H), 7.42-7.37 (m, 1H), 7.30 (d, J=7.2 Hz, 1H), 3.98 (d, J=1.2 Hz, 3H), 2.75-2.67 (m, 2H), 1.56 (s, 9H), 1.30-1.25 (m, 3H).

### Step 3: Synthesis of Intermediate 1-5

To a mixture of intermediate 1-4 (17.6 g, 47.90 mmol, 1 eq) in MeOH (60 mL), THF (60 mL) and H₂O (50 mL) was added LiOH H₂O (6.03 g, 143.70 mmol, 3 eq). The mixture was stirred at 25° C. for 1 hr. The reaction mixture was concentrated under reduced pressure and adjusted to pH=7 with HCl (3 M). Then the mixture was filtered and the filter cake was washed with H₂O (10 mL). The cake was dried under reduce pressure. The crude product was used in the next step without further purification. Intermediate 1-5 (20.67 g, crude) was obtained as a white solid. LCMS (Method D): Rt=0.464 min, [M+Na]⁺=365.0. ¹H NMR (400 MHz, METHANOL-d₄) δ=8.96 (s, 1H), 8.43 (s, 1H), 7.56-7.47 (m, 2H), 7.41 (t, J=7.6 Hz, 1H), 7.28 (d, J=7.2 Hz, 1H), 2.78-2.71 (m, 2H), 1.55 (s, 9H), 1.30 (t, J=7.6 Hz, 3H).

### Step 4: Synthesis of Intermediate 1-7

To a mixture of intermediate 1-5 (1 g, 2.92 mmol, 1 eq) and intermediate 1-6 (1.83 g, 2.92 mmol, 1 eq, HCl salt) in DMF (10 mL) was added EDCI (1.68 g, 8.76 mmol, 3 eq), HOAt (397.53 mg, 2.92 mmol, 408.57 μL, 1 eq) and NMM (1.48 g, 14.60 mmol, 1.61 mL, 5 eq). The mixture was stirred at 60° C. for 3 hr. The reaction was poured into H₂O (10 mL) and extracted with DCM (10 mL*3). The combined organic layers were dried over Na₂SO₄, filtered and concentrated under reduced pressure to give the crude product. The residue was purified by flash silica gel chromatography (ISCO; 40 g SepaFlash Silica Flash Column, Eluent of 0˜70% Ethyl acetate/Methanol gradient @40 mL/min) and concentrated under reduced pressure to give a residue. Intermediate 1-7 (2 g, 2.06 mmol, 70.42% yield, 94% purity) was obtained as a brown solid. LCMS (Method D): Rt=0.467 min, [M+H]⁺=914.5.

### Step 5: Synthesis of Intermediate 1-8

To a solution of intermediate 1-7 (2 g, 2.19 mmol, 1 eq) was added HCl/dioxane (2 M, 20 mL, 18.28 eq). The mixture was stirred at 25° C. for 1 hr. The mixture was concentrated under reduced pressure to give a residue. The crude product was used in the next step without further purification. Intermediate 1-8 (2.39 g, HCl salt) was obtained as a yellow solid. LCMS (Method D): Rt=0.386 min, [M+H]⁺=814.4.

### Step 6: Synthesis of I-367

To a solution of intermediate 1-8 (200 mg, 245.71 μmol, 1 eq) in DCM (2 mL) was added DIEA (95.27 mg, 737.14 μmol, 128.40 μL, 3 eq) and intermediate 1-9 (37.63 mg, 368.57 μmol, 34.62 μL, 1.5 eq). The mixture was stirred at 40° C. for 16 hr. The reaction mixture was concentrated under reduced pressure to give a residue. The crude product was purified by reversed-phase HPLC (Neutral condition) followed by lyophilization to give a residue. I-367 (292.33 mg, 341.51 μmol, 12.64% yield) was obtained as a white solid. LCMS (Method): Rt=1.581 min, [M+Na]=878.7 ¹H NMR (400 MHz, METHANOL-d₄) δ=8.62 (d, J=2.0 Hz, 1H), 8.37 (d, J=7.6 Hz, 1H), 8.29 (s, 1H), 7.98-7.93 (m, 1H), 7.90-7.81 (m, 2H), 7.53-7.47 (m, 3H), 7.44-7.36 (m, 2H), 7.31 (d, J=7.6 Hz, 1H), 7.16 (t, J=8.8 Hz, 1H), 4.73-4.50 (m, 2H), 4.39 (s, 2H), 3.80 (s, 1H), 3.75-3.66 (m, 4H), 3.58-3.50 (m, 2H), 3.35 (s, 1H), 3.27 (s, 1H), 3.21 (s, 1H), 3.12 (t, J=12.4 Hz, 1H), 2.93-2.85 (m, 1H), 2.77-2.71 (m, 2H), 2.60-2.42 (m, 8H), 2.29-2.22 (m, 2H), 2.17 (s, 3H), 1.95-1.86 (m, 2H), 1.77-1.70 (m, 1H), 1.29 (t, J=7.6 Hz, 5H). ¹⁹F NMR (376 MHz, METHAN OL-d₄) δ=−120.800.

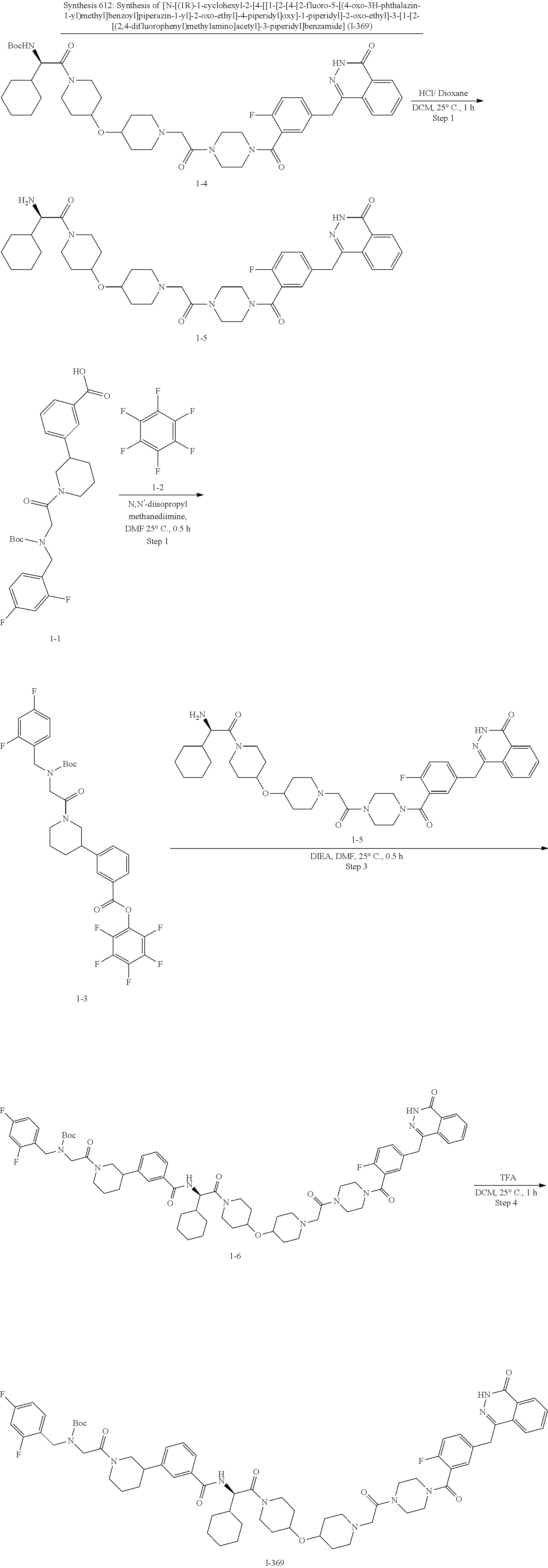

### Step 1: Synthesis of Intermediate 1-5.

To a mixture of intermediate 1-4 (5 g, 6.02 mmol, 1 eq) in DCM (25 mL) was added HCl/dioxane (2 M, 25.00 mL, 8.30 eq) at 25° C., and the mixture was stirred at 25° C. for 1 h. The reaction mixture was concentrated to give the crude product. The crude product was used for the next step directly without purification. Intermediate 1-5 (5 g, crude, HCl salt) was obtained as a yellow solid. LCMS (Method D): Retention time: 0.322 min, [M+H]⁺=730.4.

### Step 2: Synthesis of Intermediate 1-3.

To a mixture of intermediate 1-1 (4 g, 8.19 mmol, 1 eq) in DMF (40 mL) was added intermediate 1-2 (1.51 g, 8.19 mmol, 1 eq) and N,N′-diisopropylmethanediimine (1.55 g, 12.28 mmol, 1.90 mL, 1.5 eq) at 25° C. and the mixture was stirred at 25° C. for 0.5 h. The reaction mixture was diluted with H₂O (50 mL), extracted with DCM (50 mL×3), the combined organic layers were dried over Na₂SO₄, filtered and concentrated to give a residue. The residue was purified by column chromatography (SiO₂, PE/EA=1/0 to 1/1) and concentrated to give the product. Intermediate 1-3 (4.7 g, 7.18 mmol, 87.69% yield) was obtained as a white solid. LCMS (Method D): Retention time: 0.529 min, [M+H]⁺=655.1.

### Step 3: Synthesis of Intermediate 1-6.

To a mixture of intermediate 1-3 (4 g, 6.11 mmol, 1 eq) in DMF (40 mL) was added intermediate 1-5 (5.15 g, 6.72 mmol, 1.1 eq, HCl salt) and DIEA (1.58 g, 12.22 mmol, 2.13 mL, 2 eq) at 25° C., and the mixture was stirred at 25° C. for 0.5 h. The reaction mixture was diluted with H₂O (40 mL) and filtered, the filter cake was washed by H₂O (20 mL×3) and concentrated to give a residue. The residue was purified by column chromatography (SiO₂, EA/MeOH=1/0 to 7/3) and concentrated to give the product. Intermediate 1-5 (5.3 g, 4.42 mmol, 72.25% yield) was obtained as a yellow solid. LCMS (Method D): Retention time: 0.438 min, [M+H]⁺=1200.4.

### Step 4: Synthesis of I-369.

To a mixture of intermediate 1-5 (4.8 g, 4.00 mmol, 1 eq) in DCM (40 mL) was added TFA (12.28 g, 107.70 mmol, 8 mL, 26.93 eq) at 25° C., and the mixture was stirred at 25° C. for 0.5 h. The reaction mixture was concentrated to give a crude product. Another 2 g of intermediate 1-5 was synthesized using 2.2 g of intermediate 1-1 according to the disclosed method, followed by treating with TFA in DCM as described here. The crude product from the two batches were combined and purified by reversed phase chromatography (0.1% TFA condition) and lyophilized to give the product. I-369 (4.95 g, 4.01 mmol, 69.65% yield, 98.49% purity, TFA salt) was obtained as a white solid. LCMS: Retention time: 0.347 min, [M+H]⁺=1100.7 (Method D). SFC: Retention time: 2.800 min, 3.095 min. ¹H NMR (400 MHz, METHANOL-d₄) δ=8.37 (d, J=7.2 Hz, 1H), 7.98-7.93 (m, 1H), 7.91-7.80 (m, 2H), 7.77 (s, 1H), 7.74 (t, J=7.2 Hz, 1H), 7.52-7.41 (m, 3H), 7.40-7.35 (m, 1H), 7.23-7.13 (m, 2H), 6.56 (d, J=9.6 Hz, 1H), 6.53-6.47 (m, 1H), 5.10-5.01 (m, 1H), 4.58 (s, 2H), 4.55-4.47 (m, 1H), 4.39 (s, 2H), 4.15-4.04 (m, 2H), 3.94-3.86 (m, 2H), 3.78 (d, J=4.0 Hz, 4H), 3.76-3.65 (m, 7H), 3.63-3.54 (m, 3H), 3.53-3.49 (m, 1H), 3.22 (s, 3H), 2.97-2.87 (m, 2H), 2.83-2.67 (m, 2H), 2.54-2.46 (m, 2H), 2.46-2.39 (m, 2H), 2.27-2.19 (m, 2H), 2.14-2.00 (m, 3H), 1.90-1.73 (m, 4H), 1.66-1.53 (m, 2H), 1.48-1.39 (m, 6H), 1.35-1.28 (m, 2H), 1.27-1.20 (m, 1H). ¹⁹F NMR (376 MHz, METHANOL-d₄) δ=−77.2, −108.4, −112.9, −120.7.

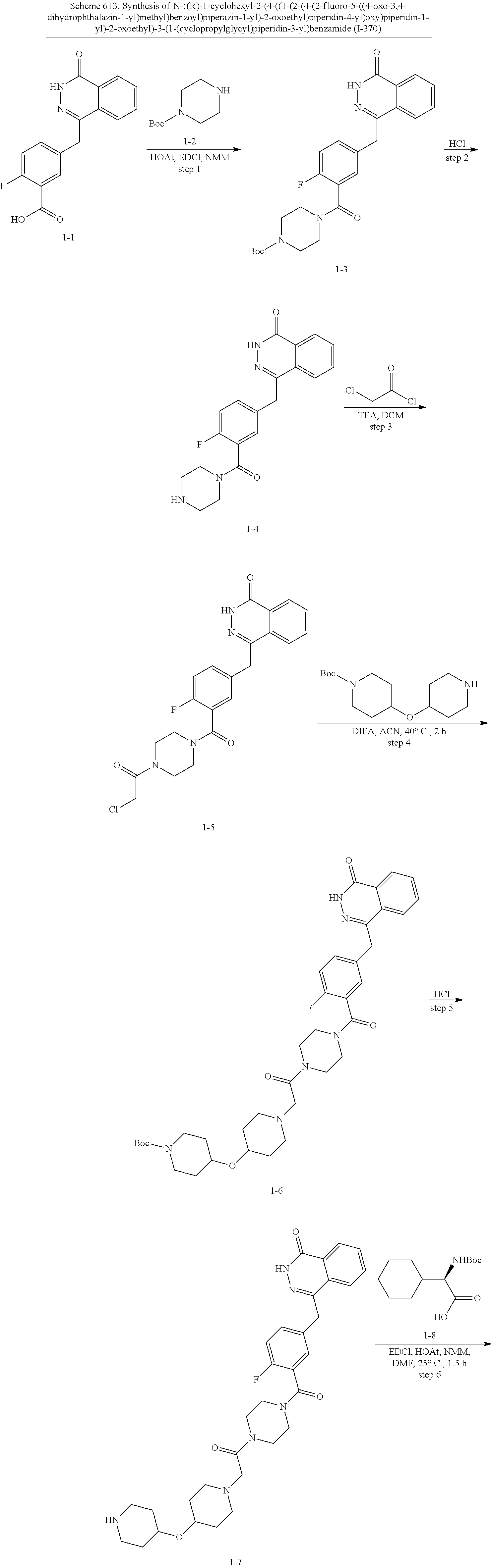

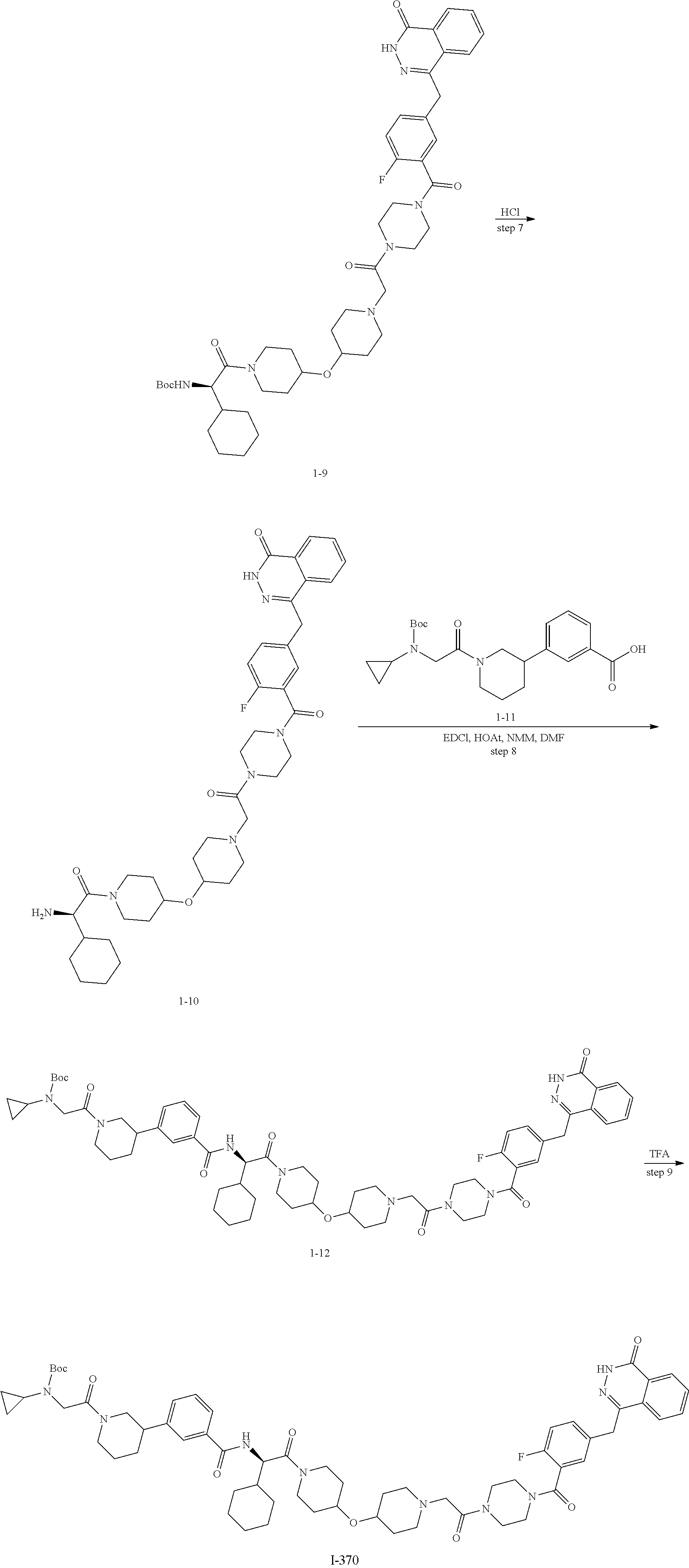

### Step 1: Synthesis of Intermediate 1-3

To a solution of Intermediate 1-1 (18 g, 60.35 mmol, 1 eq) and Intermediate 1-2 (13.49 g, 72.42 mmol, 1.2 eq) in DMF (180 mL) was added EDCI (23.14 g, 120.70 mmol, 2 eq), HOAt (8.21 g, 60.35 mmol, 8.44 mL, 1 eq) and NMM (30.52 g, 301.74 mmol, 33.17 mL, 5 eq). The mixture was stirred at 25° C. for 1 hr. To the reaction mixture was added H₂O (200 mL), the mixture was filtered, and the filter cake was washed with H₂O (150 mL*3). The filter cake was dried under reduced pressure to give a residue. The crude was used for the next step. Intermediate 1-3 (33 g, crude) was obtained as a white solid. LCMS (Method D): Retention time: 0.376 min, (M+H)=933.3.

### Step 2: Synthesis of Intermediate 1-4

A solution of Intermediate 1-3 (33 g, 70.74 mmol, 1 eq) in HCl/dioxane (2 M, 330 mL, 9.33 eq) was stirred at 25° C. for 1 hr. The reaction mixture was concentrated under reduced pressure to give a residue. The crude was used for the next step. Intermediate 1-4 (30 g, crude, HCl salt) was obtained as a white solid. LCMS (Method D): Retention time: 0.253 min, (M+H)=733.3. ¹H NMR (400 MHz, METHANOL-d₄) δ=8.39-8.25 (m, 1H), 7.97-7.90 (m, 1H), 7.89-7.77 (m, 2H), 7.55-7.44 (m, 1H), 7.42-7.40 (m, 1H), 7.15 (t, J=9.2 Hz, 1H), 4.37 (s, 2H), 3.98 (t, J=4.8 Hz, 2H), 3.61-3.55 (m, 2H), 3.29-3.28 (m, 2H), 3.16 (s, 2H). ¹⁹F NMR (376 MHz, METHANOL-d₄) δ=−120.6.

### Step 3: Synthesis of Intermediate 1-5

To a solution of Intermediate 1-4 (26 g, 70.96 mmol, 1 eq) in DCM (26 mL) was added TEA (35.90 g, 354.82 mmol, 49.39 mL, 5 eq) and 2-chloroacetyl chloride (16.03 g, 141.93 mmol, 11.30 mL, 2 eq). The mixture was stirred at 0° C. for 1 hr. To the reaction mixture was added H₂O (50 mL) and then it was extracted with DCM (50 mL*3), the combined organic phase was washed, dried by Na₂SO₄, filtered and concentrated. The residue was purified by flash silica gel chromatography (ISCO; 330 g SepaFlash Silica Flash Column, Eluent of 0˜100% Methanol/Dichloromethaneethergradient @150 mL/min) and the eluent was concentrated to give product. Intermediate 1-5 (18.5 g, 41.77 mmol, 58.87% yield) was obtained as a white solid. LCMS (Method D): Retention time: 0.320 min, (M+H)=443.0. ¹H NMR (400 MHz, DMSO-d₆) δ=12.59 (s, 1H), 8.26 (d, J=7.6 Hz, 1H), 8.05-7.72 (m, 3H), 7.51-7.31 (m, 2H), 7.24 (t, J=9.2 Hz, 1H), 4.47-4.27 (m, 4H), 3.71-3.57 (m, 2H), 3.54 (s, 2H), 3.39 (s, 2H), 3.24-3.15 (m, 2H). ¹⁹F NMR (376 MHz, DMSO-d₆) δ=−120.0.

### Step 4: Synthesis of Intermediate 1-6

To a solution of tert-butyl 4-(4-piperidyloxy)piperidine-1-carboxylate (900 mg, 3.16 mmol, 1 eq) and Intermediate 1-5 (1.40 g, 3.16 mmol, 1 eq) in ACN (9 mL) was added DIEA (409.00 mg, 3.16 mmol, 551.21 μL, 1 eq). The mixture was stirred at 40° C. for 1 hr. The reaction mixture was filtered and the filtrate was purified by flash silica gel chromatography. The residue was purified by flash silica gel chromatography (ISCO; 40 g SepaFlash Silica Flash Column, Eluent of 0˜100% Ethyl acetate/Petroleum ethergradient @50 mL/min) and the eluent was concentrated to give product. Intermediate 1-6 (1.7 g, 2.46 mmol, 77.76% yield) was obtained as a white solid. LCMS (Method D): Retention time: 0.330 min, (M+H)=691.3. ¹H NMR (400 MHz, CHLOROFORM-d) δ=10.84-10.48 (m, 1H), 8.67-8.32 (m, 1H), 7.85-7.58 (m, 3H), 7.33 (s, 2H), 7.05 (t, J=9.2 Hz, 1H), 4.30 (s, 2H), 3.86-3.51 (m, 9H), 3.49-2.99 (m, 8H), 2.75 (s, 2H), 2.35-2.12 (m, 2H), 1.91-1.73 (m, 5H), 1.59-1.50 (m, 2H), 1.45 (s, 9H). ¹⁹F NMR (376 MHz, CHLOROFORM-d) δ=−117.6.

### Step 5: Synthesis of Intermediate 1-7

A solution of Intermediate 1-6 (1.4 g, 2.03 mmol, 1 eq) in HCl/dioxane (2 M, 14.00 mL, 13.82 eq) was stirred at 25° C. for 1 hr. The reaction mixture was concentrated under reduced pressure to give a residue. The crude was used for the next step. Intermediate 1-7 (1.4 g, crude, HCl salt) was obtained as a white solid. LCMS (Method D): Retention time: 0.208 min, (M+H)=591.2.

### Step 6: Synthesis of Intermediate 1-9

To a solution of Intermediate 1-7 (1.1 g, 1.75 mmol, 1 eq, HCl salt) and Intermediate 1-8 (451.34 mg, 1.75 mmol, 1 eq) in DMF (11 mL) was added EDCI (672.48 mg, 3.51 mmol, 2 eq), HOAt (238.74 mg, 1.75 mmol, 245.36 μL, 1 eq) and NMM (887.05 mg, 8.77 mmol, 964.18 μL, 5 eq). The mixture was stirred at 25° C. for 1 hr. The reaction mixture was filtered and the filtrate was purified by flash silica gel chromatography (ISCO; 40 g SepaFlash Silica Flash Column, Eluent of 0˜100% Ethyl acetate/Petroleum ether gradient @40 mL/min) and the eluent was concentrated to give product. Intermediate 1-9 (2 g) was obtained as a white solid. LCMS (Method D): Retention time: 0.375 min, (M+H)=830.4. SFC: Retention time: 1.103 min. ¹H NMR (400 MHz, CHLOROFORM-d) δ=10.84-10.48 (m, 1H), 8.67-8.32 (m, 1H), 7.85-7.58 (m, 3H), 7.33 (s, 2H), 7.05 (t, J=9.2 Hz, 1H), 4.30 (s, 2H), 3.86-3.51 (m, 9H), 3.49-2.99 (m, 8H), 2.75 (s, 2H), 2.35-2.12 (m, 2H), 1.91-1.73 (m, 5H), 1.59-1.50 (m, 2H), 1.45 (s, 9H). ¹⁹F NMR (376 MHz, CHLOROFORM-d) δ=−120.8.

### Step 7: Synthesis of Intermediate 1-10

A solution of Intermediate 1-9 (1.3 g, 1.57 mmol, 1 eq) in HCl/dioxane (2 M, 13.00 mL, 16.60 eq) was stirred at 25° C. for 1 hr. The reaction mixture was concentrated under reduced pressure to give a residue. The crude was used in the next step. Intermediate 1-10 (1.2 g, 1.57 mmol, 99.98% yield, HCl salt) was obtained as a yellow solid. LCMS (Method D): Retention time: 0.271 min, (M+H)=730.4.

### Step 8: Synthesis of Intermediate 1-12

To a solution of Intermediate 1-10 (600 mg, 782.94 μmol, 1 eq, HCl salt) and Intermediate 1-11 (315.12 mg, 782.94 μmol, 1 eq) in DMF (6 mL) was added EDCI (300.18 mg, 1.57 mmol, 2 eq), HOAt (106.57 mg, 782.94 μmol, 109.52 μL, 1 eq) and NMM (395.96 mg, 3.91 mmol, 430.39 μL, 5 eq). The mixture was stirred at 25° C. for 1 hr. The reaction mixture was filtered and the filtrate was purified by flash silica gel chromatography (ISCO; 40 g SepaFlash Silica Flash Column, Eluent of 0˜100% Ethyl acetate/Petroleum ether gradient @40 mL/min) and the eluent was concentrated to give product. Intermediate 1-12 (700 mg, 628.17 μmol, 80.23% yield) was obtained as a white solid. LCMS (Method D): Retention time: 0.424 min, (M+H)=1114.5. SFC: Retention time: 1.893, 5.365 min. ¹H NMR (400 MHz, METHANOL-d₄) δ=8.57 (d, J=4.0 Hz, 2H), 8.37 (d, J=7.6 Hz, 1H), 8.25 (d, J=8.4 Hz, 2H), 7.99-7.68 (m, 2H), 7.54-7.42 (m, 1H), 7.39-7.37 (m, 2H), 7.17 (s, 1H), 4.94-4.90 (m, 2H), 4.59-4.50 (m, 1H), 4.39 (s, 1H), 4.29-4.16 (m, 1H), 4.13-3.98 (m, 1H), 3.96-3.65 (m, 11H), 3.58-3.31 (m, 16H), 3.29-3.13 (m, 10H), 2.86-2.69 (m, 7H), 2.12-1.60 (m, 8H), 1.45 (s, 4H), 1.36-1.04 (m, 3H), 0.79-0.53 (m, 2H). ¹⁹F NMR (376 MHz, METHANOL-d₄) δ=−120.8.

### Step 9: Synthesis of I-370

To a solution of Intermediate 1-12 (600 mg, 538.43 μmol, 1 eq) in DCM (0.6 mL) was added TFA (1.84 g, 16.15 mmol, 1.20 mL, 30.00 eq). The mixture was stirred at 25° C. for 1 hr. The reaction mixture was concentrated under reduced pressure to give a residue. The crude product was purified by reversed-phase HPLC (0.1% TFA condition), and the eluent was concentrated to remove ACN and lyophilized to give product. I-370 (217.54 mg, 189.96 μmol, 35.28% yield, 98.524% purity, TFA salt) was obtained as a white solid. LCMS (Method D): Retention time: 0.333 min, (M+H)=1014.5. SFC: Retention time: 3.091, 3.580 min. ¹H NMR (400 MHz, METHANOL-d₄) δ=8.37 (d, J=7.6 Hz, 1H), 7.99-7.91 (m, 1H), 7.91-7.67 (m, 4H), 7.59-7.30 (m, 4H), 7.17 (t, J=8.8 Hz, 1H), 4.99-4.90 (m, 2H), 4.61-4.37 (m, 3H), 4.34-4.25 (m, 2H), 4.25-4.10 (m, 2H), 4.07-3.92 (m, 2H), 3.89-3.61 (m, 7H), 3.60-3.40 (m, 5H), 3.40-3.32 (m, 4H), 3.27-3.04 (m, 2H), 3.03-2.86 (m, 1H), 2.86-2.72 (m, 2H), 2.29-2.13 (m, 1H), 2.12-1.99 (m, 4H), 1.98-1.83 (m, 6H), 1.77 (s, 2H), 1.74-1.58 (m, 4H), 1.35-1.21 (m, 3H), 1.17-1.04 (m, 2H), 1.01-0.81 (m, 4H). ¹⁹F NMR (376 MHz, METHANOL-d₄) δ=−77.2, −120.7.

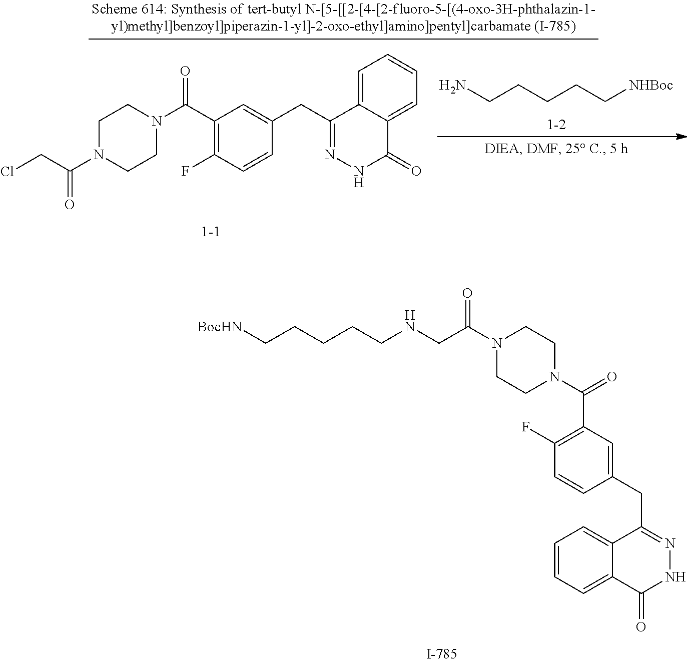

To a mixture of intermediate 1-1 (8 g, 18.06 mmol, 1 eq) and intermediate 1-2 (3.65 g, 18.06 mmol, 3.76 mL, 1 eq) in DMF (100 mL) was added DIEA (4.67 g, 36.13 mmol, 6.29 mL, 2 eq). The mixture was stirred at 25° C. for 5 hr. The reaction was poured into H₂O (100 mL) and extracted with EA (150 mL*3). The combined organic layers were dried over Na₂SO₄, filtered and concentrated under reduced pressure to give the crude product. The residue was purified by flash silica gel chromatography (ISCO; 220 g SepaFlash Silica Flash Column, Eluent of 0˜40% Ethyl acetate/Methanol gradient @100 mL/min) and concentrated under reduced pressure to give a residue. The residue was further purified by prep-HPLC (column: Phenomenex luna C18 (250*70 mm, 10 um); mobile phase: [water(NH₄HCO₃)-ACN]; gradient: 15%-45% B over 20 min) followed by lyophilization to give a residue. I-785 (2.7 g, 3.83 mmol, 21.18% yield, 92.775% purity, FA salt) was obtained as a white solid. LCMS (Method D): Rt=0.299 min, (M+H)=609.2. ¹H NMR (400 MHz, METHANOL-d₄) δ=8.37 (d, J=7.2 Hz, 1H), 8.31 (s, 1H), 7.97-7.92 (m, 1H), 7.90-7.81 (m, 2H), 7.53-7.48 (m, 1H), 7.40-7.34 (m, 1H), 7.17 (t, J=9.2 Hz, 1H), 4.38 (s, 2H), 4.14-4.04 (m, 2H), 3.87-3.64 (m, 4H), 3.54 (d, J=3.6 Hz, 2H), 3.38 (s, 1H), 3.34 (s, 1H), 3.07-3.00 (m, 4H), 1.78-1.69 (m, 2H), 1.55-1.48 (m, 2H), 1.43 (s, 11H). ¹⁹F NMR (376 MHz, METHANOL-d₄) δ=−120.7.

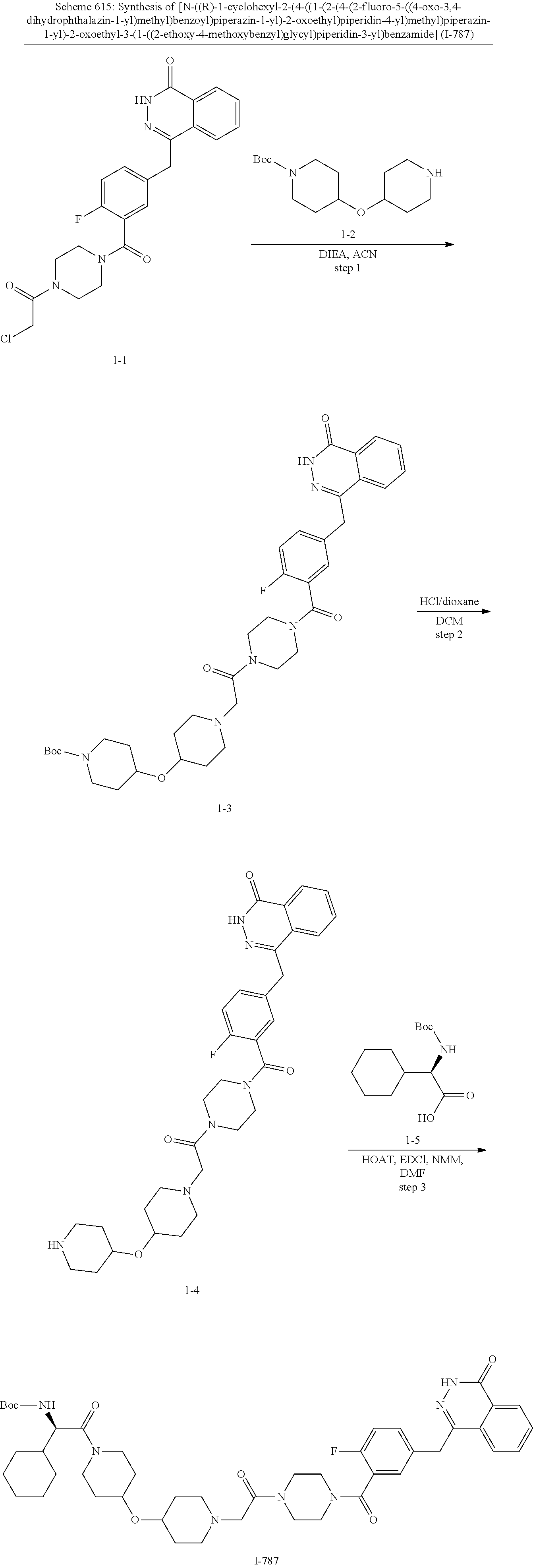 [SMILES: O=C(CCl)N1CCN(C(=O)c2cc(Cc3n[nH]c(=O)c4ccccc34)ccc2F)CC1 ; CC(C)(C)OC(=O)N1CCC(OC2CCNCC2)CC1 ; CC(C)(C)OC(=O)N1CCC(OC2CCN(CC(=O)N3CCN(C(=O)c4cc(Cc5n[nH]c(=O)c6ccccc56)ccc4F)CC3)CC2)CC1 ; O=C(CN1CCC(OC2CCNCC2)CC1)N1CCN(C(=O)c2cc(Cc3n[nH]c(=O)c4ccccc34)ccc2F)CC1 ; CC(C)(C)OC(=O)N[C@@H](C(=O)O)C1CCCCC1 ; CC(C)(C)OC(=O)N[C@@H](C(=O)N1CCC(OC2CCN(CC(=O)N3CCN(C(=O)c4cc(Cc5n[nH]c(=O)c6ccccc56)ccc4F)CC3)CC2)CC1)C1CCCCC1]

### Step 1: Synthesis of Intermediate 1-3.

To a solution of Intermediate 1-2 (4.6 g, 16.17 mmol, 1 eq) and Intermediate 1-1 in ACN (45 mL) was added DIEA (6.27 g, 48.52 mmol, 8.45 mL, 3 eq). The mixture was stirred at 40° C. for 1 hr. The reaction mixture was diluted with H₂O (20 mL) and extracted with EA (20 mL*3). The combined organic layers were dried over Na₂SO₄, filtered and concentrated under reduced pressure to give a residue. The residue was purified by flash silica gel chromatography (ISCO; 120 g SepaFlash Silica Flash Column, Eluent of 10% EA/MeOH gradient @60 mL/min). The eluent was concentrated under reduced pressure to give Intermediate 1-3 (9.7 g, 13.47 mmol, 83.29% yield, 95.943% purity) as a brown solid. LCMS (Method D): Retention time: 0.343 min, (M+H)=691.4. ¹H NMR (400 MHz, CHLOROFORM-d) δ=8.37 (d, J=8.4 Hz, 1H), 8.01-7.80 (m, 3H), 7.54-7.34 (m, 2H), 7.16 (m, 1H), 4.69-4.52 (m, 2H), 4.39 (s, 2H), 3.80-3.46 (m, 10H), 3.28-3.18 (m, 2H), 3.12 (s, 2H), 2.77 (s, 2H), 2.25 (m, 2H), 2.02 (s, 4H), 1.91-1.76 (m, 4H), 1.62-1.55 (m, 1H), 1.45 (s, 9H).

### Step 2: Synthesis of Intermediate 1-4.

To a solution of Intermediate 1-3 (9.5 g, 13.75 mmol, 1 eq) in DCM (36 mL) was added HCl/dioxane (2 M, 95.00 mL). The mixture was stirred at 25° C. for 0.5 hr. The reaction mixture was concentrated under reduced pressure to give a residue. Without further purification, it was used directly for the next reaction. Intermediate 1-4 (7.9 g, 10.84 mmol, 78.83% yield, 86.065% purity, HCl salt) was obtained as a white solid. LCMS (Method D): Retention time: 0.243 min, (M+H)=591.4.

### Step 3: Synthesis of I-787.

To a solution of Intermediate 1-5 (7 g, 11.16 mmol, 1 eq, HCl salt) and Intermediate 1-4 (2.87 g, 11.16 mmol, 1.00 eq) in DMF (100 mL) was added HOAt (1.52 g, 11.16 mmol, 1.56 mL, 1 eq), EDCI (6.42 g, 33.48 mmol, 3 eq) and NMM (5.64 g, 55.81 mmol, 6.14 mL, 5 eq). The mixture was stirred at 25° C. for 1 hr. The reaction mixture was diluted with H₂O (200 mL) and extracted with EA (100 mL*3). The combined organic layers were washed by brine (100 mL*3), dried over Na₂SO₄, filtered and concentrated under reduced pressure to give a residue. The residue was purified by flash silica gel chromatography (ISCO; 120 g SepaFlash Silica Flash Column, Eluent of 40% EA/PE gradient @80 mL/min). The eluent concentrated under reduced pressure to give the crude product (8 g) as a white solid. 500 mg of the crude product was purified again by flash silica gel chromatography (ISCO; 20 g SepaFlash Silica Flash Column, Eluent of 0-10% Methanol/Ethyl acetate gradient @60 mL/min), and the eluent was concentrated and lyophilized to give a product. I-787 (300 mg, 357.06 μmol, 59.27% yield, 98.787% purity) was obtained as a white solid. LCMS (Method D): Retention time: 0.391 min, (M+H)=830.4. SFC: Retention time: 0.682, 0.994 min. ¹HNMR (400 MHz, METHANOL-d4) δ=8.37 (d, J=7.6 Hz, 1H), 7.99-7.92 (m, 1H), 7.90-7.81 (m, 2H), 7.55-7.35 (m, 2H), 7.23-7.11 (m, 1H), 4.39 (s, 3H), 4.04-3.64 (m, 8H), 3.62-3.45 (m, 4H), 3.39 (d, J=11.2 Hz, 2H), 3.27-3.19 (m, 2H), 2.77 (s, 2H), 2.33-2.18 (m, 2H), 1.88 (s, 4H), 1.75 (d, J=7.6 Hz, 3H), 1.68-1.55 (m, 6H), 1.43 (s, 9H), 1.35-1.11 (m, 4H), 1.10-0.98 (m, 2H). ¹⁹FNMR (376 MHz, METHANOL-d4) δ=−120.8.

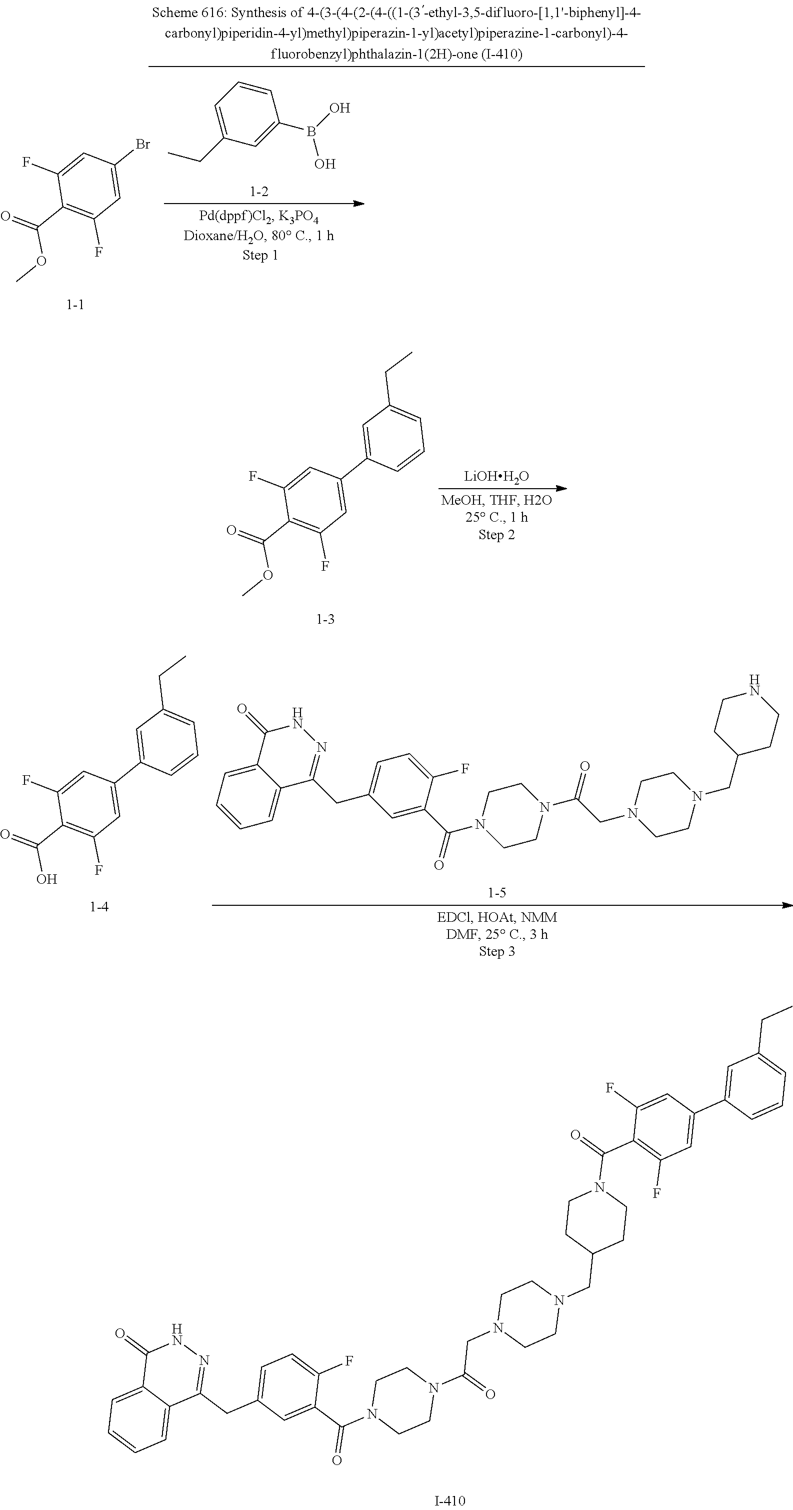

### Step 1: Synthesis of Intermediate 1-3

To a solution of intermediate 1-1 (200 mg, 796.73 μmol, 1 eq) and intermediate 1-2 (143.40 mg, 956.08 μmol, 1.2 eq) in H₂O (0.4 mL) and dioxane (2 mL) was added K₃PO₄ (507.36 mg, 2.39 mmol, 3 eq) and Pd(dppf)Cl₂ (51.93 mg, 79.67 μmol, 0.1 eq). The mixture was stirred at 80° C. for 1 hr. The reaction mixture was concentrated under reduced pressure to give a crude product. The crude product was used in the next step without purification. Intermediate 1-3 (210 mg, crude) was obtained as white solid. LCMS (Method D): Rt=0.461 min, [M+H]⁺=277.1.

### Step 2: Synthesis of Intermediate 1-4

To a solution of intermediate 1-3 (190 mg, 687.71 μmol, 1 eq) in THF (0.7 mL), H₂O (0.7 mL) and MeOH (0.7 mL) was added LiOH·H₂O (86.58 mg, 2.06 mmol, 3 eq). The mixture was stirred at 25° C. for 1 hr. The pH of the reaction mixture was adjusted by HCl (1 M) to pH 6.0-7.0, and then the reaction mixture was quenched by addition of H₂O (5 mL) at 25° C. and extracted with DCM (5 mL*3). The combined organic layer was extracted and concentrated under reduced pressure to give a crude product. The crude product was used in the next step without purification. Intermediate 1-4 (200 mg, crude) was obtained as a white solid. LCMS (Method D): Rt=0.518 min, (M+H)=887.5.

### Step 3: Synthesis of I-410

To a solution of intermediate 1-4 (50 mg, 190.66 μmol, 1 eq) and intermediate 1-5 (134.92 mg, 215.47 μmol, 1.13 eq, HCl salt) in DMF (0.5 mL) was added HOAt (12.98 mg, 95.33 μmol, 13.34 μL, 0.5 eq), EDCI (73.10 mg, 381.31 μmol, 2 eq) and NMM (96.42 mg, 953.29 μmol, 104.81 μL, 5 eq). The mixture was stirred at 25° C. for 3 hrs. The reaction mixture was concentrated under reduced pressure to give a crude product. The crude product was purified by reversed-phase HPLC (0.10% FA condition) and lyophilized to give the product. I-410 (28.1 mg, 31.93 μmol, 16.75% yield, FA salt) was obtained as a white solid. LCMS (Method D): Rt=0.388 min, (M+H)=834.4. ¹H NMR (400 MHz, METHANOL-d₄) δ=8.44 (s, 1H), 8.37 (d, J=7.6 Hz, 1H), 7.98-7.92 (m, 1H), 7.91-7.86 (m, 1H), 7.85-7.80 (m, 1H), 7.55-7.44 (m, 3H), 7.43-7.38 (m, 2H), 7.38-7.33 (m, 2H), 7.29 (d, J=7.6 Hz, 1H), 7.16 (t, J=9.2 Hz, 1H), 4.90 (s, 2H), 4.70 (d, J=12.8 Hz, 1H), 4.65-4.51 (m, 1H), 4.39 (s, 2H), 3.84-3.59 (m, 5H), 3.56-3.48 (m, 2H), 3.37 (s, 2H), 3.20 (t, J=13.2 Hz, 1H), 2.92 (t, J=12.8 Hz, 1H), 2.758-2.701 (m, 3H), 2.69-2.54 (m, 6H), 2.44-2.31 (m, 2H), 2.01-1.87 (m, 2H), 1.86-1.77 (m, 1H), 1.28 (t, J=7.6 Hz, 3H), 1.25-1.10 (m, 2H). ¹⁹F NMR (376 MHz, METHANOL-d4) δ=−115.36, −115.42, −120.8.

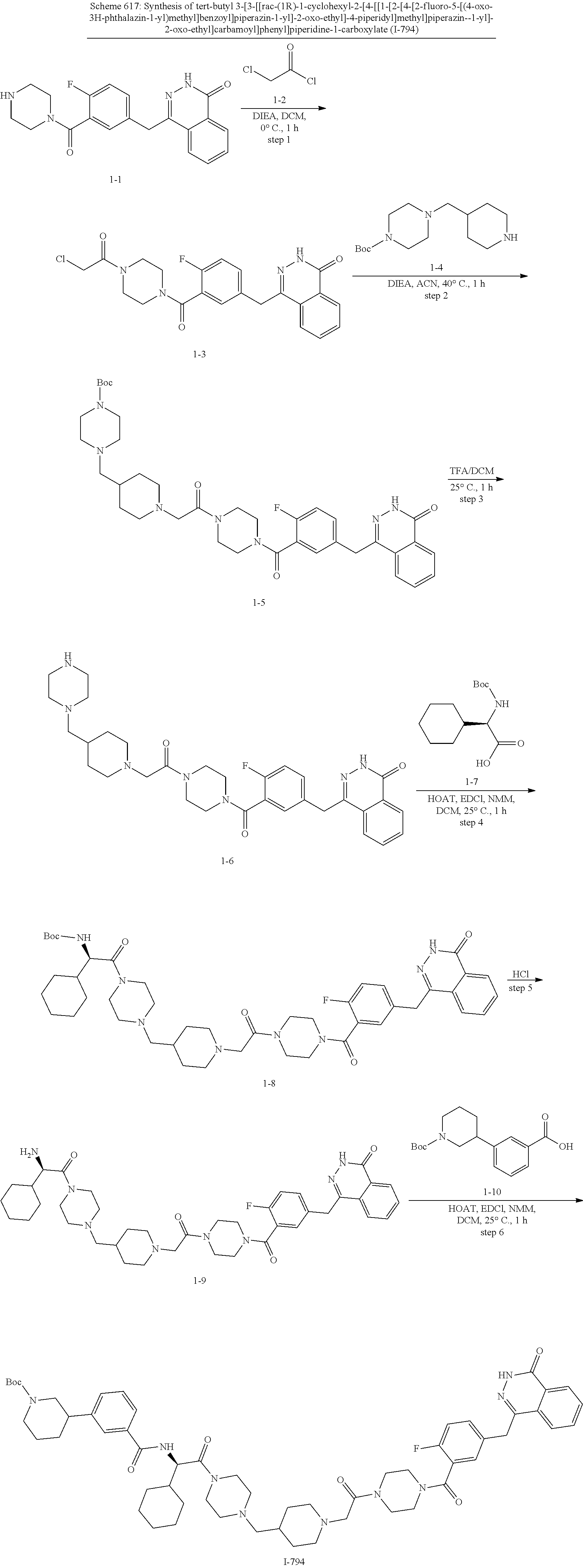

### Step 1: Synthesis of Intermediate 1-3

To a solution of Intermediate 1-1 (36 g, 98.26 mmol, 1 eq) in DCM (360 mL) was added DIEA (63.49 g, 491.28 mmol, 85.57 mL, 5 eq) at 0° C. Then Intermediate 1-2 (14.43 g, 127.73 mmol, 10.17 mL, 1.3 eq) was dropwise into the mixture and it was stirred at 0° C. for 1 hr. The reaction mixture was diluted with water (120 mL) and extracted with DCM (40 mL×3). The combined organic layers were washed with brine (40 mL×3) and dried over sodium sulfate, filtered and concentrated under reduced pressure to give a residue. The residue was purified by flash silica gel chromatography (ISCO; 330 g SepaFlash Silica Flash Column, Eluent of 0˜10% Methanol: Ethyl acetate gradient @100 mL/min). The eluent was concentrated under reduced pressure to give Intermediate 1-3 (26.2 g, 43.78 mmol, 44.55% yield, 74% purity) as a brown gum. LCMS (Method D): Rt: 0.333 min, (M+H)=443.0. ¹H NMR (400 MHz, CHLOROFORM-d) δ=10.78-10.64 (m, 1H), 8.56-8.41 (m, 1H), 7.82-7.70 (m, 3H), 7.34 (d, J=6.0 Hz, 2H), 7.05-7.03 (m, 1H), 4.30 (s, 2H), 3.91-3.72 (m, 2H), 3.72-3.57 (m, 3H), 3.54-3.46 (m, 1H), 3.44-3.28 (m, 2H), 3.11-3.09 (m, 1H), 3.02-2.84 (m, 1H). ¹⁹F NMR (376 MHz, CHLOROFORM-d) δ=−117.642.

### Step 2: Synthesis of Intermediate 1-5

To a solution of Intermediate 1-3 (25 g, 56.45 mmol, 1 eq) and Intermediate 1-4 (16.00 g, 56.45 mmol, 1 eq) in ACN (300 mL) was added DIEA (21.89 g, 169.35 mmol, 29.50 mL, 3 eq). The mixture was stirred at 40° C. for 1 hr. The crude product was triturated with ACN (200 mL) and filtered to give a filter cake. The filter cake was the pure desired product. The filtrate was not pure but contained some product. Then the filtrate was concentrated to give a residue. The residue was diluted with water (210 mL) and extracted with ethyl acetate (70 mL×3). The combined organic layers were washed with brine (70 mL×3) and dried over sodium sulfate, filtered and concentrated under reduced pressure to give Intermediate 1-5 (38 g, crude) as a brown oil. LCMS (Method D): Rt: 0.263 min, (M+H)=690.3.

### Step 3: Synthesis of Intermediate 1-6

To a solution of Intermediate 1-5 (20 g, 28.99 mmol, 1 eq) in DCM (200 mL) was added TFA (61.40 g, 538.49 mmol, 40.00 mL, 18.57 eq). The mixture was stirred at 25° C. for 0.5 hr. The mixture was concentrated under reduced pressure to give Intermediate 1-6 (18 g, 25.58 mmol, 88.22% yield, TFA salt) as a yellow oil. LCMS (Method D): Rt: 0.215 min, (M+H)=590.3.

### Step 4: Synthesis of Intermediate 1-8

To a solution of Intermediate 1-7 (6.58 g, 25.58 mmol, 1 eq) in DCM (200 mL) was added EDCI (14.71 g, 76.73 mmol, 3 eq) dropwise, HOAt (3.48 g, 25.58 mmol, 3.58 mL, 1 eq) and NMM (12.94 g, 127.89 mmol, 14.06 mL, 5 eq) at 25° C. and the mixture was stirred for 0.5 hr. Intermediate 1-6 (18 g, 25.58 mmol, 1 eq, TFA salt) was added into mixture and it was stirred at 25° C. for 0.5 hr. The reaction mixture was diluted with water (120 mL) and extracted with DCM (40 mL×3). The combined organic layers were washed with brine (40 mL×3) and dried over sodium sulfate, filtered and concentrated under reduced pressure to give a residue. The residue was purified by prep-HPLC (column: Welch Ultimate XB-Diol 250*50*10 um; mobile phase: [Hexane-EtOH]; gradient: 15%-47% B over 16 min). The eluent was concentrated under reduced pressure to give a residue. Intermediate 1-8 (12.5 g, 10.83 mmol, 42.33% yield, 71.8% purity) was obtained as a white solid. LCMS (Method H): Rt: 0.674 min, (M+H)=829.4. ¹H NMR (400 MHz, CHLOROFORM-d) δ=8.50-8.36 (m, 1H), 7.83-7.70 (m, 3H), 7.33 (s, 2H), 7.30-7.043 (m, 1H), 5.28 (d, J=8.0 Hz, 1H), 4.45-4.37 (m, 1H), 4.28 (s, 2H), 4.02-3.47 (m, 13H), 3.44-3.23 (m, 4H), 2.68-2.23 (m, 6H), 2.02-1.91 (m, 2H), 1.80-1.69 (m, 7H), 1.43 (s, 9H), 1.29-0.98 (m, 7H). ¹⁹F NMR (376 MHz, METHANOL-d4) δ=−117.6. SFC: Rt: 1.176 min.

### Step 5: Synthesis of Intermediate 1-9

To a solution of Intermediate 1-8 (10 g, 12.06 mmol, 1 eq) in DCM (100 mL) was added HCl/dioxane (2 M, 6.03 mL, 1 eq). The mixture was stirred at 25° C. for 0.5 hr. The mixture was concentrated under reduced pressure to give Intermediate 1-9 (8.9 g, 11.63 mmol, 96.40% yield, HCl salt) as a yellow oil. LCMS (Method D): Rt: 0.242 min, (M+H)=729.3.

### Step 6: Synthesis of I-794

To a solution of Intermediate 1-10 in DCM (200 mL) was added EDCI (11.27 g, 58.80 mmol, 3 eq), NMM (9.91 g, 97.99 mmol, 10.77 mL, 5 eq) and HOAt (2.67 g, 19.60 mmol, 2.74 mL, 1 eq) at 25° C. and the mixture was stirred for 0.5 hr. Intermediate 1-9 (15 g, 19.60 mmol, 1 eq, HCl salt) was added into the mixture and it was stirred at 25° C. for 0.5 hr. The reaction mixture was diluted with water (120 mL) and extracted with ethyl acetate (40 mL×3). The combined organic layers were washed with brine (40 mL×3) and dried over sodium sulfate, filtered and concentrated under reduced pressure to give a residue. The crude product was purified by reversed-phase HPLC (column: CD02-Waters Xbidge BEH C18 150*25*10 um; mobile phase: [water (NH₄HCO₃)-ACN]; gradient: 53%-83% B over 10 min) and the eluent was concentrated under reduced pressure to remove ACN and then lyophilized to give I-794 (10.6 g, 10.43 mmol, 53.22% yield, 100% purity) as a white solid. LCMS (Method D): Rt: 0.390 min, (M+H)=1016.5. ¹H NMR (400 MHz, METHANOL-d4) δ=8.38 (d, J=7.6 Hz, 1H), 8.00-7.93 (m, 1H), 7.88-7.83 (m, 2H), 7.75 (s, 1H), 7.70 (d, J=7.6 Hz, 1H), 7.54-7.34 (m, 4H), 7.19-7.17 (m, 1H), 4.91 (d, J=8.8 Hz, 2H), 4.85 (s, 1H), 4.59 (s, 1H), 4.40 (s, 2H), 4.12 (d, J=12.8 Hz, 2H), 3.90-3.79 (m, 2H), 3.74 (s, 1H), 3.69-3.65 (m, 1H), 3.62-3.50 (m, 3H), 3.35 (s, 1H), 3.28-3.18 (m, 2H), 2.98-2.78 (m, 4H), 2.77-2.69 (m, 1H), 2.55-2.34 (m, 4H), 2.24-2.20 m, 2H), 2.14-1.98 (m, 3H), 1.96-1.85 (m, 2H), 1.83-1.66 (m, 8H), 1.64-1.53 (m, 2H), 1.47 (s, 9H), 1.37-1.08 (m, 8H). ¹⁹F NMR (376 MHz, METHANOL-d4) δ=−120.8. SFC: Rt: 8.373 min, 9.823 min.

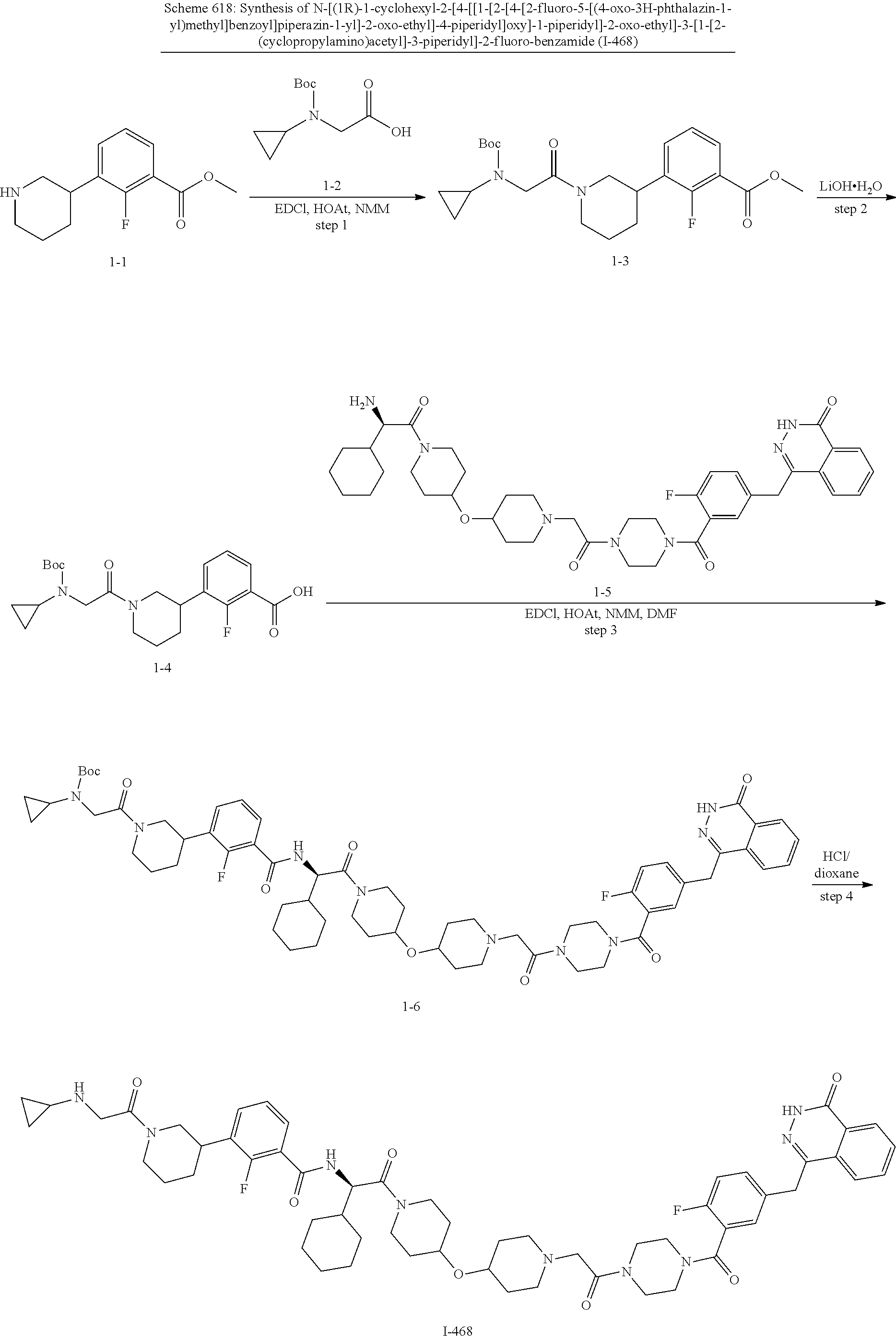

### Step 1: Synthesis of Intermediate 1-3

A mixture of Intermediate 1-1 (0.4 g, 1.46 mmol, 1 eq, HCl salt), Intermediate 1-2 (314.54 mg, 1.46 mmol, 1 eq) and HOAt (198.90 mg, 1.46 mmol, 204.42 μL, 1 eq) in DMF (4 mL) was added EDCI (560.26 mg, 2.92 mmol, 2 eq) and NMM (739.05 mg, 7.31 mmol, 803.31 μL, 5 eq). The mixture was stirred at 25° C. for 2 hrs. The mixture was diluted with water (10 mL) and extracted with EtOAc (10 mL*3). The organic layer was dried with anhydrous Na₂SO₄, filtered and concentrated under vacuum. The crude product was purified by reversed-phase chromatography (0.1% NH₃·H₂O condition) and concentrated under vacuum. Intermediate 1-3 (570 mg, 1.31 mmol, 89.77% yield) was obtained as yellow oil. LCMS (Method G): Rt=0.685 min, [M+H-Boc]⁺=335.2.

### Step 2: Synthesis of Intermediate 1-4

To a solution of Intermediate 1-3 (570 mg, 1.21 mmol) in THF (3 mL), H₂O (1.5 mL) and MeOH (1.5 mL) was added LiOH·H₂O (152.35 mg, 3.63 mmol, 3 eq). The mixture was stirred at 25° C. for 2 hr. The mixture was adjusted to pH=5 with 2N HCl and concentrated under vacuum. The crude product was purified by reversed-phase chromatography (0.1% FA condition) and concentrated under vacuum to remove MeCN and dried by lyophilization. Intermediate 1-4 (550 mg, crude) was obtained as a white solid. LCMS (Method E): Rt=0.509 min, [M+H]⁺=421.3.

### Step 3: Synthesis of Intermediate 1-6

A mixture of Intermediate 1-4 (0.2 g, 475.65 μmol, 1 eq), Intermediate 1-5 (364.51 mg, 475.65 μmol, 1 eq, HCl salt) and HOAt (64.74 mg, 475.65 μmol, 66.54 μL, 1 eq) in DMF (3 mL) was added EDCI (182.37 mg, 951.31 μmol, 2 eq) and NMM (240.56 mg, 2.38 mmol, 261.48 μL, 5 eq). The mixture was stirred at 25° C. for 2 hrs. The mixture was poured into water (6 mL) and filtered and the filter cake was concentrated under vacuum. This residue used in the next step without purification. Intermediate 1-6 (550 mg, crude) was obtained as a yellow solid. LCMS (Method): Rt=2.065 min, [M+H]⁺=1132.8.

### Step 4: Synthesis of I-468

A mixture of Intermediate 1-6 (290 mg, 256.11 μmol, 1 eq) in DCM (2 mL) was added HCl/dioxane (4 M, 1.00 mL, 15.62 eq) and it was stirred at 25° C. for 0.5 hr. The mixture was concentrated under vacuum. The residue was combined with another lot of material (100 mg) and to purified. The residue was purified by Prep-HPLC (column: Phenomenex luna C18 150*25 mm*10 um; mobile phase: [water (HCl)-ACN]; gradient: 13%-43% B over 10 min) and dried by lyophilization. The residue was further purified by Prep-HPLC (column: Phenomenex luna C18 150*25 mm*10 um; mobile phase: [water (HCl)-ACN]; gradient: 13%-43% B over 10 min) and dried by lyophilization. The residue was further purified by Prep-HPLC (column: Phenomenex luna C18 150*25 mm*10 um; mobile phase: [water (HCl)-ACN]; gradient: 12%-42% B over 20 min) and dried by lyophilization. I-468 (204.43 mg, 192.50 μmol, 75.16% yield, 97.24% purity, HCl salt) was obtained as white solid. LCMS (Method E): Rt=0.454 min, [M+H]⁺=1032.6. SFC: Rt=3.357 min. ¹H NMR (400 MHz, METHANOL-d₄) δ=8.37 (d, J=7.6 Hz, 1H), 7.99-7.92 (m, 1H), 7.92-7.80 (m, 2H), 7.66-7.47 (m, 3H), 7.42-7.33 (m, 1H), 7.28-7.26 (m, 1H), 7.19-7.17 (m, 1H), 5.05-4.94 (m, 1H), 4.58 (br d, J=13.2 Hz, 1H), 4.39 (s, 2H), 4.36-4.31 (m, 1H), 4.31-4.18 (m, 3H), 4.07-3.99 (m, 1H), 3.98-3.90 (m, 1H), 3.81 (br d, J=12.4 Hz, 5H), 3.75-3.61 (m, 2H), 3.58-3.44 (m, 4H), 3.43-3.33 (m, 4H), 3.28-3.03 (m, 3H), 2.93-2.74 (m, 2H), 2.30-2.17 (m, 1H), 2.11-2.01 (m, 4H), 2.00-1.49 (m, 14H), 1.34-1.08 (m, 5H), 0.98-0.87 (m, 4H). ¹⁹F NMR (377 MHz, METHANOL-d₄) δ=−120.750, −121.537, −121.918.

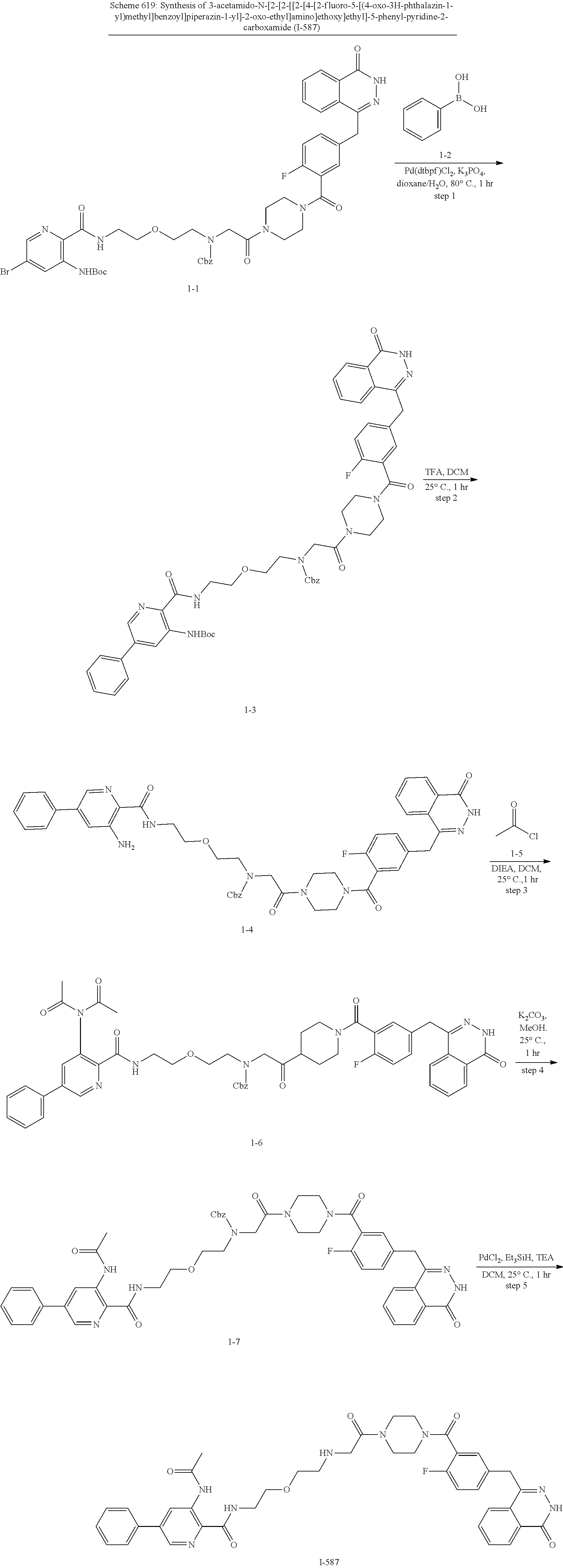

### Step 1: Synthesis of Intermediate 1-3.

To a solution of Intermediate 1-1 (200 mg, 211.91 μmol, 1 eq) in H₂O (0.4 mL) and 1,4-dioxane (1 mL) was added Intermediate 1-2 (31.01 mg, 254.29 μmol, 1.2 eq), K₃PO₄ (134.94 mg, 635.72 μmol, 3 eq) and Pd(dtbpf)Cl₂ (13.81 mg, 21.19 μmol, 0.1 eq). The mixture was stirred at 80° C. for 1 hr under N₂. The reaction mixture was diluted with H₂O (1 mL) and extracted with EA (2 mL*2). The combined organic layers were dried over Na₂SO₄, filtered and concentrated under reduced pressure to give a residue. The residue was purified by flash silica gel chromatography (ISCO; 20 g SepaFlash Silica Flash Column, Eluent of 0˜100% Ethyl acetate/Petroleum ether gradient @60 mL/min). The eluent was concentrated under reduced pressure to give Intermediate 1-3 (190 mg, 198.24 gmol, 93.55% yield, 98.184% purity) as a white solid. LCMS (Method D): Retention time: 0.543 min, [M+H]⁺=941.2.

### Step 2: Synthesis of Intermediate 1-4.

A solution of Intermediate 1-3 (120 mg, 127.52 gmol, 1 eq) in DCM (1.2 mL) and TFA (0.25 mL) was stirred at 25° C. for 1 hr. The mixture was concentrated in vacuum to give Intermediate 1-4 (120 mg, 125.67 μmol, 98.54% yield, 100% purity, TFA salt) as a yellow solid. LCMS (Method D): Retention time: 0.463 min, [M+H]⁺=841.2.

### Step 3: Synthesis of Intermediate 1-6.

To a solution of Intermediate 1-4 (100 mg, 104.72 gmol, 1 eq, TFA salt) in DCM (1 mL) was added DIEA (40.60 mg, 314.16 μmol, 54.72 μL, 3 eq) and Intermediate 1-5 (8.22 mg, 104.72 μmol, 7.45 L, 1 eq). The mixture was stirred at 25° C. for 1 hr. The mixture was concentrated in vacuum to give Intermediate 1-6 (110 mg, crude) as a yellow oil. The product was used in the next step without further purification. LCMS (Method D): Retention time: 0.496 min, [M+H]⁺=925.4.

### Step 4: Synthesis of Intermediate 1-7.

To a solution of Intermediate 1-6 (110 mg, 118.92 gmol, 1 eq) in MeOH (1 mL) was added K₂CO₃ (49.31 mg, 356.77 μmol, 3 eq). The mixture was stirred at 25° C. for 1 hr. The reaction mixture was diluted with H₂O (1 mL) and extracted with EA (1 mL*2). The combined organic layers were dried over Na₂SO₄, filtered and concentrated under reduced pressure to give Intermediate 1-7 (100 mg, 109.16 μmol, 91.79% yield, 96.377% purity) as a yellow solid. The product was used in the next step without further purification. LCMS (Method D): Retention time: 0.482 min, [M+H]⁺=883.3.

### Step 5: Synthesis of I-587.

To a solution of Intermediate 1-7 ((80 mg, 90.61 gmol, 1 eq) in DCM (2 mL) was added PdCl₂ (4.82 mg, 27.18 μmol, 0.3 eq) and TEA (18.34 mg, 181.21 gmol, 25.22 μL, 2 eq). The reaction mixture was degassed with N₂. Then a solution of Et₃SiH (42.14 mg, 362.43 gmol, 57.89 μL, 4 eq) in DCM (1 mL) was added dropwise at 10° C. The mixture was stirred at 25° C. for 1 h. The mixture was filtered and the filtrate was concentrated in vacuum. Then the mixture diluted with NaHCO₃ (2 mL) and extracted with CH₂Cl₂ (2 mL*2). The combined organic layers were dried over Na₂SO₄, filtered and concentrated under reduced pressure to give a residue. The crude product was purified by reversed-phase chromatography (0.1% FA condition). The eluent was concentrated under reduced pressure to remove ACN and then lyophilized to give the product. I-587 (21.79 mg, 27.02 gmol, 29.82% yield, 98.551% purity, FA salt) was obtained as a white solid. LCMS (Method D): Retention time: 0.370 min, [M+H]⁺=749.3. ¹H NMR (400 MHz, METHANOL-d₄) δ=9.30-9.22 (m, 1H), 8.59-8.52 (m, 1H), 8.39-8.31 (m, 1H), 7.95-7.91 (m, 1H), 7.89-7.79 (m, 2H), 7.70-7.64 (m, 2H), 7.55-7.40 (m, 4H), 7.35-7.28 (m, 1H), 7.18-7.10 (m, 1H), 4.39-4.32 (m, 2H), 4.00-3.85 (m, 2H), 3.80-3.61 (m, 9H), 3.49-3.42 (m, 2H), 3.30-3.20 (m, 3H), 3.19-3.11 (m, 2H), 2.28-2.20 (m, 3H). ¹⁹FNMR (376 MHz, METHANOL-d₄) δ=−120.652.

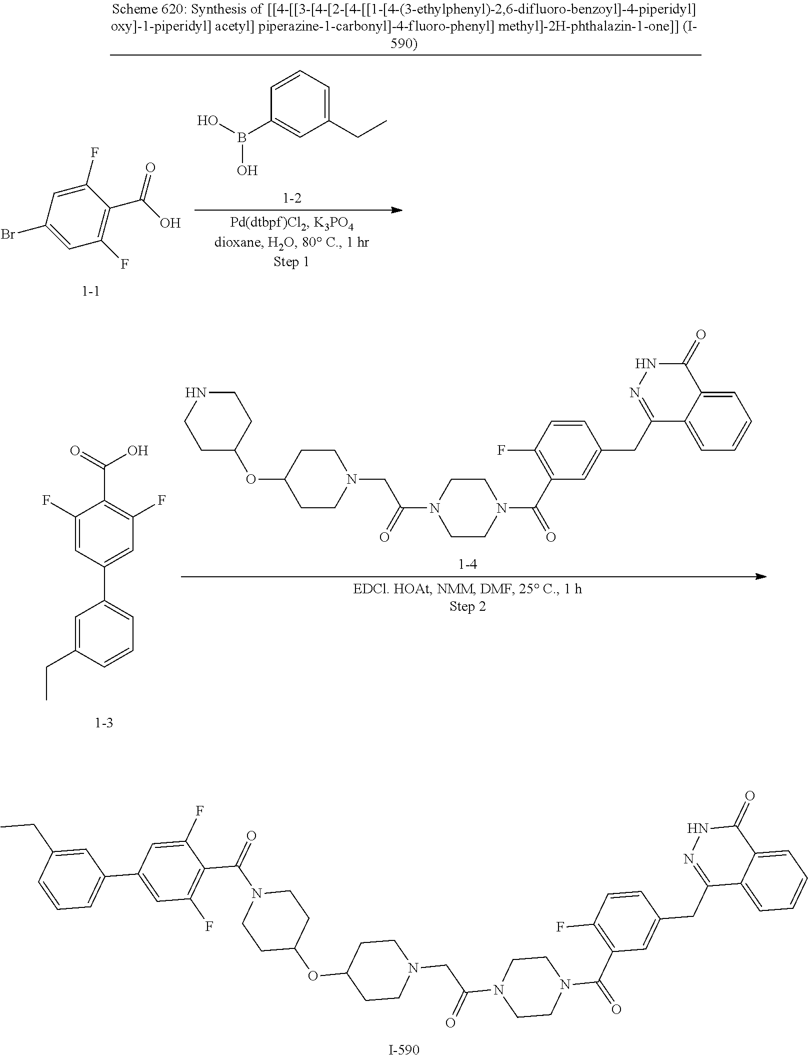 [SMILES: O=C(O)c1c(F)cc(Br)cc1F ; CCc1cccc(B(O)O)c1 ; CCc1cccc(-c2cc(F)c(C(=O)O)c(F)c2)c1 ; O=C(CN1CCC(OC2CCNCC2)CC1)N1CCN(C(=O)c2cc(Cc3n[nH]c(=O)c4ccccc34)ccc2F)CC1 ; CCc1cccc(-c2cc(F)c(C(=O)N3CCC(OC4CCN(CC(=O)N5CCN(C(=O)c6cc(Cc7n[nH]c(=O)c8ccccc78)ccc6F)CC5)CC4)CC3)c(F)c2)c1]

### Step 1: Synthesis of Intermediate 1-3

A mixture of intermediate 1-1 (100 mg, 421.94 μmol, 1 eq), intermediate 1-2 (75.94 mg, 506.33 μmol, 1.2 eq), ditert-butyl (cyclopenta-1,4-dien-1-yl) phosphane; dichloropalladium; iron (27.50 mg, 42.19 μmol, 0.1 eq) and K₃PO₄ (268.69 mg, 1.27 mmol, 3 eq) in dioxane (1 mL) and H₂O (0.2 mL) was degassed and purged with N₂ 3 times, and then the mixture was stirred at 80° C. for 1 hr under N₂ atmosphere. The reaction mixture was poured into H₂O (2 mL) and extracted with EA (2 mL*3). The combined organic layers were dried over Na₂SO₄, filtered and concentrated under reduced pressure to give a residue. The residue was purified by flash silica gel chromatography (ISCO; 12 g SepaFlashSilica Flash Column, Eluent of 0˜100% DCM/MeOH@36 mL/min, DCM/MeOH=10:1, Rf=0.3) and the eluent was concentrated under reduced pressure to give a product. Intermediate 1-3 (60 mg, crude) was obtained as a brown gum. Mass Found: LCMS (Method D): Rt=0.457 min, (M+H)=263.1.

### Step 2: Synthesis of I-590

To a solution of intermediate 1-3 (10 mg, 38.13 μmol, 1 eq) and intermediate 1-4 (23.91 mg, 38.13 μmol, 1 eq, HCl salt) in DMF (0.1 mL) was added EDCI (21.93 mg, 114.39 μmol, 3 eq), HOAt (5.19 mg, 38.13 μmol, 5.33 μL, 1 eq) and NMM (19.28 mg, 190.66 μmol, 20.96 μL, 5 eq). The mixture was stirred at 25° C. for 0.5 hr. The reaction mixture was poured into H₂O (2 mL) and extracted with EA (2 mL*3). The combined organic layers were dried over Na₂SO₄, filtered and concentrated under reduced pressure to give a residue. The residue was purified by prep-HPLC (column: CD03-Welch Xtimate C18 150*25*5 um; mobile phase: [water (FA)-ACN]; gradient: 25%-55% B over 10 min) and the eluent was concentrated to remove MeCN and then lyophilized. I-590 (16.62 mg, 19.91 μmol, 52.20% yield, 100% purity) was obtained as a yellow solid. LCMS (Method D): Rt=0.418 min, (M+H)=835.2. ¹H NMR (400 MHz, METHANOL-d₄) δ=8.44-8.31 (m, 1H), 7.94 (d, J=4.4 Hz, 1H), 7.91-7.80 (m, 2H), 7.53-7.44 (m, 3H), 7.43-7.33 (m, 4H), 7.29 (d, J=7.6 Hz, 1H), 7.17 (t, J=8.8 Hz, 1H), 4.39 (s, 2H), 4.11-4.02 (m, 1H), 3.84-3.73 (m, 3H), 3.71-3.55 (m, 6H), 3.54-3.44 (m, 3H), 3.04-2.88 (m, 2H), 2.73 (q, J=7.6 Hz, 2H), 2.68-2.46 (m, 2H), 2.08-1.78 (m, 5H), 1.76-1.56 (m, 4H), 1.31-1.26 (m, 5H). F NMR (400 MHz, METHANOL-d4) δ=−115.398, −115.458, −120.787.

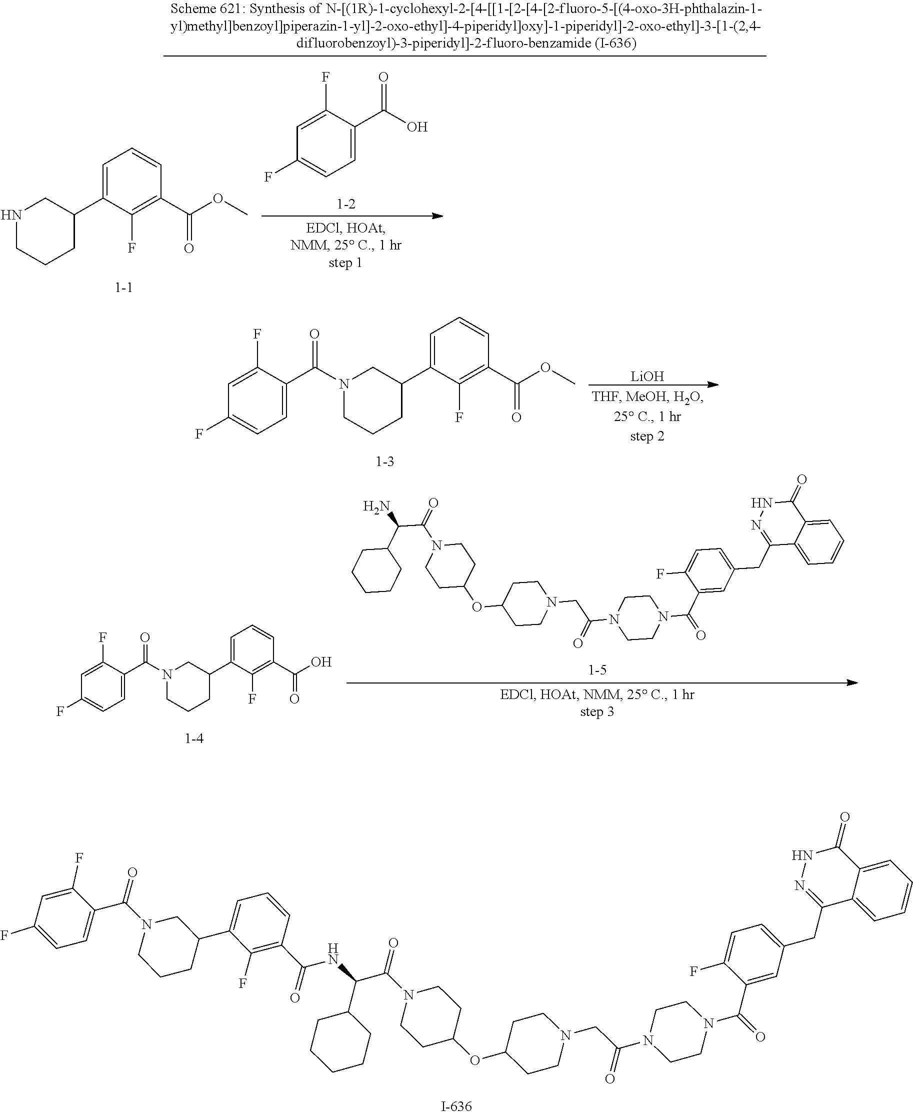

### Step 1: Synthesis of Intermediate 1-3

To a solution of intermediate 1-1 (500 mg, 1.83 mmol, 1 eq, HCl salt) and intermediate 1-2 (317.67 mg, 2.01 mmol, 1.1 eq) in DMF (5 mL) was added EDCI (1.05 g, 5.48 mmol, 3 eq), NMM (923.78 mg, 9.13 mmol, 1.00 mL, 5 eq) and HOAt (248.62 mg, 1.83 mmol, 255.52 μL, 1 eq). The mixture was stirred at 25° C. for 1 hr. The reaction mixture was extracted with DCM (15 mL*3). The combined organic layers were dried over Na₂SO₄, filtered and concentrated under reduced pressure to give a residue. The residue was purified by flash silica gel chromatography (ISCO; 20 g SepaFlash Silica Flash Column, Eluent of 0˜35% Ethyl acetate/Petroleum ether gradient @36 mL/min) and concentrated under reduced pressure to give a residue. Intermediate 1-3 (558 mg, 1.43 mmol, 78.52% yield, 97% purity) was obtained as a white solid. LCMS (Method D): Rt=0.439 min, [M+H]⁺=378.4.

### Step 2: Synthesis of Intermediate 1-4

To a solution of intermediate 1-3 (500 mg, 1.33 mmol, 1 eq) in THF (5 mL), MeOH (5 mL) and H₂O (2.5 mL) was added LiOH·H₂O (278.01 mg, 6.63 mmol, 5 eq). The mixture was stirred at 25° C. for 1 hr. The reaction mixture was concentrated under reduced pressure and adjusted to pH=4 with HCl (1 M). Then the mixture was extracted with DCM (15 mL*3). The combined organic layers were dried over Na₂SO₄, filtered and concentrated under reduced pressure to give a residue. The crude product was used in the next step without further purification. Intermediate 1-4 (510 mg, crude) was obtained as a white solid. LCMS (Method D): Rt=0.348 min, [M+H]⁺=364.0.

### Step 3: Synthesis of I-636

To a solution of Intermediate 1-4 (214.05 mg, 589.14 μmol, 1 eq) and Intermediate 1-5 (430 mg, 589.14 μmol, 1 eq) in DMF (5 mL) was added EDCI (338.81 mg, 1.77 mmol, 3 eq), NMM (297.95 mg, 2.95 mmol, 323.86 μL, 5 eq) and HOAt (80.19 mg, 589.14 μmol, 82.41 μL, 1 eq). The mixture was stirred at 25° C. for 1 hr. The reaction mixture was filtered and concentrated under reduced pressure to give a residue. The crude product was purified by reversed-phase chromatography (neutral condition) followed by lyophilization to give product. The product was further purified by reversed-phase chromatography (neutral condition) followed by lyophilization to give product. The product was further purified by flash silica gel chromatography (ISCO; 20 g SepaFlash Silica Flash Column, Eluent of 0˜9% DCM/MeOH @36 mL/min) and concentrated under reduced pressure to give a residue. To the mixture was added MeCN and H₂O, followed by lyophilization to give product. I-636 (280.64 mg, 259.77 μmol, 62.07% yield, 99.524% purity) was obtained as a white solid. LCMS (Method D): Rt=0.428 min, (M−H)=1075.2. ¹HNMR (400 MHz, METHANOL-d₄) δ=8.37 (d, J=7.6 Hz, 1H), 7.95 (d, J=7.6 Hz, 1H), 7.91-7.79 (m, 2H), 7.65-7.41 (m, 4H), 7.38 (s, 1H), 7.31-7.15 (m, 2H), 7.14-7.02 (m, 2H), 5.00-4.92 (m, 1H), 4.73 (d, J=12.0 Hz, 1H), 4.38 (s, 2H), 4.11-3.96 (m, 1H), 3.94-3.83 (m, 1H), 3.82-3.70 (m, 4H), 3.67 (s, 1H), 3.63-3.40 (m, 6H), 3.29-3.10 (m, 5H), 3.07-2.88 (m, 1H), 2.84-2.69 (m, 2H), 2.36-2.19 (m, 2H), 2.10-1.74 (m, 12H), 1.70 (d, J=10.4 Hz, 2H), 1.65-1.46 (m, 4H), 1.39-0.99 (m, 6H). ¹⁹F NMR (377 MHz, METHANOL-d₄) δ=−108.920, −113.182, −120.528.

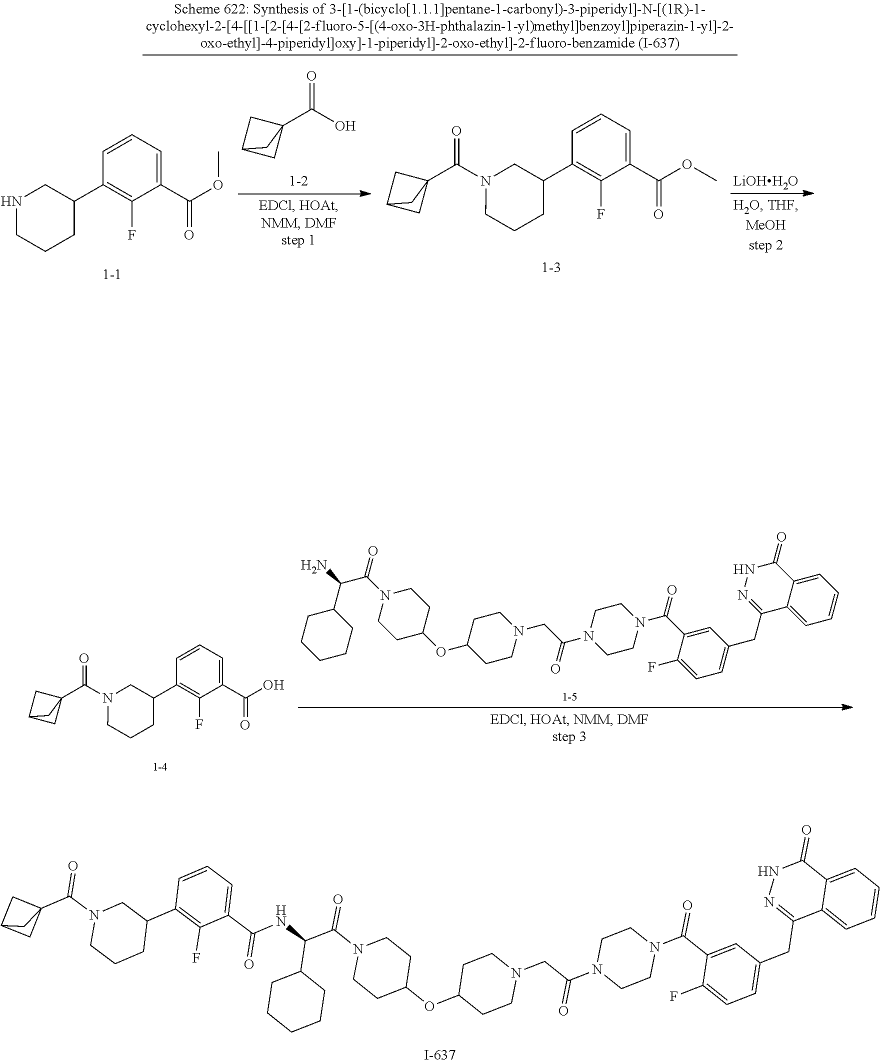

### Step 1: Synthesis of Intermediate 1-3

To a solution of intermediate 1-1 (0.5 g, 1.83 mmol, 1 eq, HCl salt) and intermediate 1-2 (204.81 mg, 1.83 mmol, 1 eq) in DMF (5 mL) was added HOAt (497.24 mg, 3.65 mmol, 511.04 μL, 2 eq), EDCI (1.75 g, 9.13 mmol, 5 eq), and NMM (1.85 g, 18.27 mmol, 2.01 mL, 10 eq). The mixture was stirred at 25° C. for 0.5 hr. The reaction mixture was diluted with water (10 mL) and extracted with DCM (10 mL*3). The combined organic layers were dried over Na₂SO₄, filtered and concentrated under reduced pressure to give a residue. The residue was purified by flash silica gel chromatography (ISCO; 40 g SepaFlash Silica Flash Column, Eluent of 0˜50% Petroleum ether/Ethyl acetate @40 mL/min). The eluent was concentrated under reduced pressure to give a residue. Intermediate 1-3 (505 mg, 1.52 mmol, 83.43% yield) was obtained as a white solid. LCMS (Method D): Rt=0.423 min, [M+H]⁺=332.1.

### Step 2: Synthesis of Intermediate 1-4

To a solution of intermediate 1-3 (0.5 g, 1.51 mmol, 1 eq) in MeOH (2 mL), THF (2 mL), and H₂O (2 mL) was added LiOH·H₂O (126.63 mg, 3.02 mmol, 2 eq). The mixture was stirred at 25° C. for 1 hr. The mixture was adjusted to pH=5 with citric acid and extracted with EA (10 mL*3). The combined organic layers were dried over Na₂SO₄, filtered and concentrated under reduced pressure to give a residue. The residue was used for the next step without further purification. Intermediate 1-4 (0.5 g, crude) was obtained as a white solid. LCMS (Method D): Rt=0.376 min, [M+H]⁺=318.2.

### Step 3: Synthesis of I-637

To a solution of intermediate 1-4 (200 mg, 630.21 μmol, 1 eq) in DMF (2 mL) was added HOAt (85.78 mg, 630.21 μmol, 88.16 μL, 1 eq), EDCI (362.44 mg, 1.89 mmol, 3 eq), NMM (318.72 mg, 3.15 mmol, 346.43 μL, 5 eq), and intermediate 1-5 (482.96 mg, 630.21 μmol, 1 eq, HCl salt). The mixture was stirred at 25° C. for 1 hr. The reaction mixture was diluted with water (10 mL) and extracted with DCM (10 mL*3). The combined organic layers were dried over Na₂SO₄, filtered and concentrated under reduced pressure to give a residue. The residue was purified by prep-HPLC (column: CD04-Welch Utimate C18 150*25*7 um;mobile phase: [water(TFA)-ACN]; gradient: 23%-53% B over 10 min). The eluent was lyophilized to give a product. The product was further purified by reversed phase chromatography (0.1% TFA condition), and the eluent was concentrated under reduced pressure to remove ACN. The eluent was adjusted to pH=10 with LiOH·H₂O (aq.) and extracted with DCM (20 mL*4). The combined organic layers were concentrated under reduced pressure to give a residue, then to the residue was added ACN with 0.1% TFA, and the mixture was lyophilized to give product. I-637 (220 mg, 192.44 μmol, 56.59% yield, TFA salt) was obtained as a white solid. LCMS (Method D): Rt=0.424 min, [M+H]⁺=1029.3. ¹H NMR (400 MHz, METHANOL-d4) δ=8.47-8.40 (m, 1H), 8.37 (d, J=7.6 Hz, 1H), 7.99-7.92 (m, 1H), 7.92-7.79 (m, 2H), 7.60-7.45 (m, 3H), 7.41-7.34 (m, 1H), 7.31-7.22 (m, 1H), 7.18 (t, J=8.8 Hz, 1H), 4.99-4.93 (m, 1H), 4.54 (d, J=11.6 Hz, 1H), 4.41-4.29 (m, 4H), 4.26-4.19 (m, 1H), 4.09-4.01 (m, 1H), 3.99-3.90 (m, 1H), 3.85-3.61 (m, 6H), 3.60-3.41 (m, 5H), 3.35 (s, 3H), 3.21-3.01 (m, 3H), 2.81-2.64 (m, 1H), 2.52-2.41 (m, 1H), 2.25-2.16 (m, 6H), 2.10-1.95 (m, 5H), 1.94-1.74 (m, 8H), 1.72-1.53 (m, 5H), 1.35-1.06 (m, 6H). ¹⁹F NMR (376 MHz, METHANOL-d4) δ=−77.41 (s), −120.70 (d), −121.46-−121.95 (m). SFC: Retention time: 3.978 min, 5.666 min.

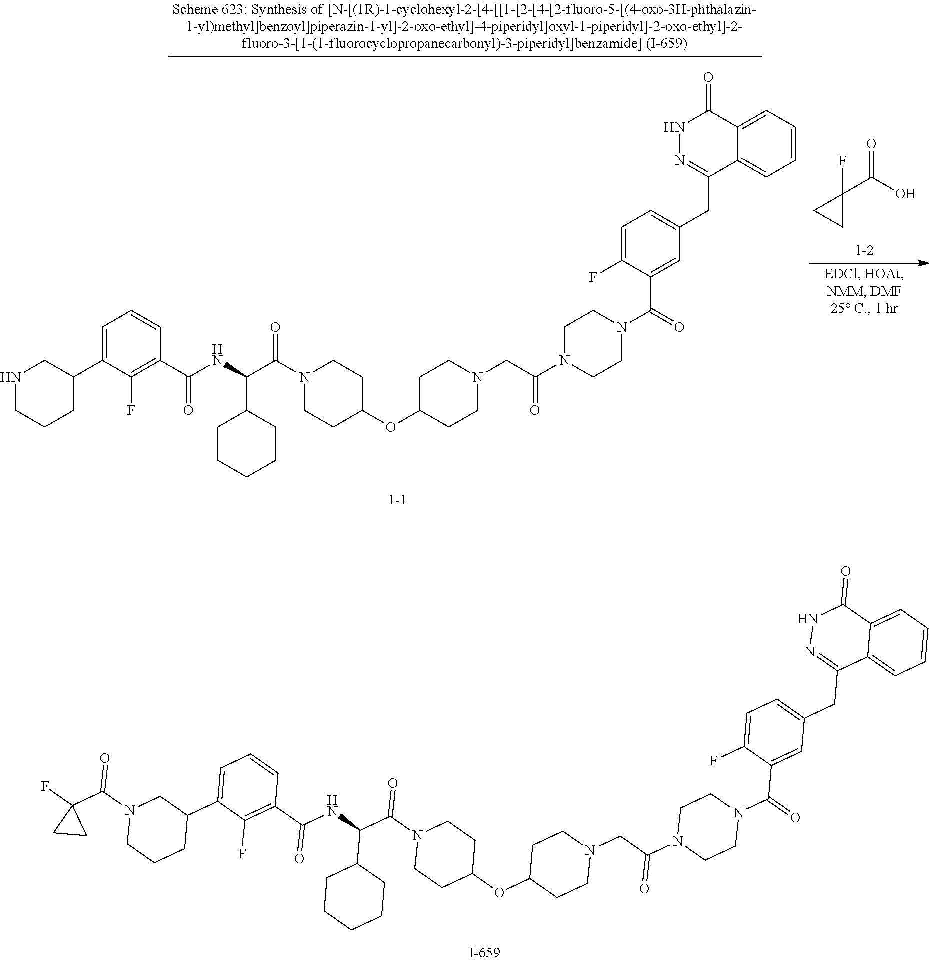 [SMILES: O=C(N[C@@H](C(=O)N1CCC(OC2CCN(CC(=O)N3CCN(C(=O)c4cc(Cc5n[nH]c(=O)c6ccccc56)ccc4F)CC3)CC2)CC1)C1CCCCC1)c1cccc(C2CCCNC2)c1F ; O=C(O)C1(F)CC1 ; O=C(N[C@@H](C(=O)N1CCC(OC2CCN(CC(=O)N3CCN(C(=O)c4cc(Cc5n[nH]c(=O)c6ccccc56)ccc4F)CC3)CC2)CC1)C1CCCCC1)c1cccc(C2CCCN(C(=O)C3(F)CC3)C2)c1F]

To a solution of intermediate 1-1 (700 mg, 720.48 μmol, 1 eq, HCl salt) in DMF (7 mL) was added intermediate 1-2 (89.99 mg, 864.58 μmol, 1.2 eq), EDCI (414.35 mg, 2.16 mmol, 3 eq), HOAt (98.07 mg, 720.48 μmol, 100.79 μL, 1 eq) and NMM (364.37 mg, 3.60 mmol, 396.06 μL, 5 eq) at 25° C., and the mixture was stirred at 25° C. for 1 hr. The reaction mixture was diluted with H₂O (10 mL), extracted with DCM (5 mL×3), and the combined organic layers were dried over Na₂SO₄, filtered and concentrated to give a residue. The residue was purified by prep-HPLC (column: CD05-Phenomenex luna C18 150*40*10 um; mobile phase: [water (FA)-ACN]; gradient: 15%-45% B over 10 min) and lyophilized to give the product. I-659 (366.48 mg, 336.54 μmol, 46.71% yield, 98.00% purity, FA salt) was obtained as a white solid. LCMS (Method D): Retention time: 0.401 min, [M+H]⁺=1021.6. SFC: Retention time: 3.142 min, 4.023 min. ¹H NMR (400 MHz, METHANOL-d4) δ=8.43 (s, 1H), 8.37 (d, J=7.2 Hz, 1H), 7.99-7.93 (m, 1H), 7.91-7.80 (m, 2H), 7.60-7.47 (m, 3H), 7.37 (t, J=6.0 Hz, 1H), 7.30-7.22 (m, 1H), 7.17 (br t, J=9.2 Hz, 1H), 4.95 (d, J=8.0 Hz, 1H), 4.53-4.42 (m, 2H), 4.39 (s, 2H), 4.09-3.99 (m, 1H), 3.96-3.83 (m, 1H), 3.80 (s, 4H), 3.71-3.56 (m, 5H), 3.56-3.41 (m, 4H), 3.40-3.33 (m, 2H), 3.27-3.22 (m, 1H), 3.20-2.95 (m, 4H), 2.82-2.57 (m, 2H), 2.09-2.02 (m, 1H), 1.93 (d, J=11.6 Hz, 4H), 1.89-1.80 (m, 4H), 1.79-1.72 (m, 3H), 1.72-1.59 (m, 4H), 1.57-1.46 (m, 1H), 1.29 (d, J=2.4 Hz, 3H), 1.25 (s, 4H), 1.17-1.06 (m, 2H). ¹⁹F NMR (376 MHz, METHANOL-d4) δ=−120.78 (s), −121.70 (s), −187.62 (s).

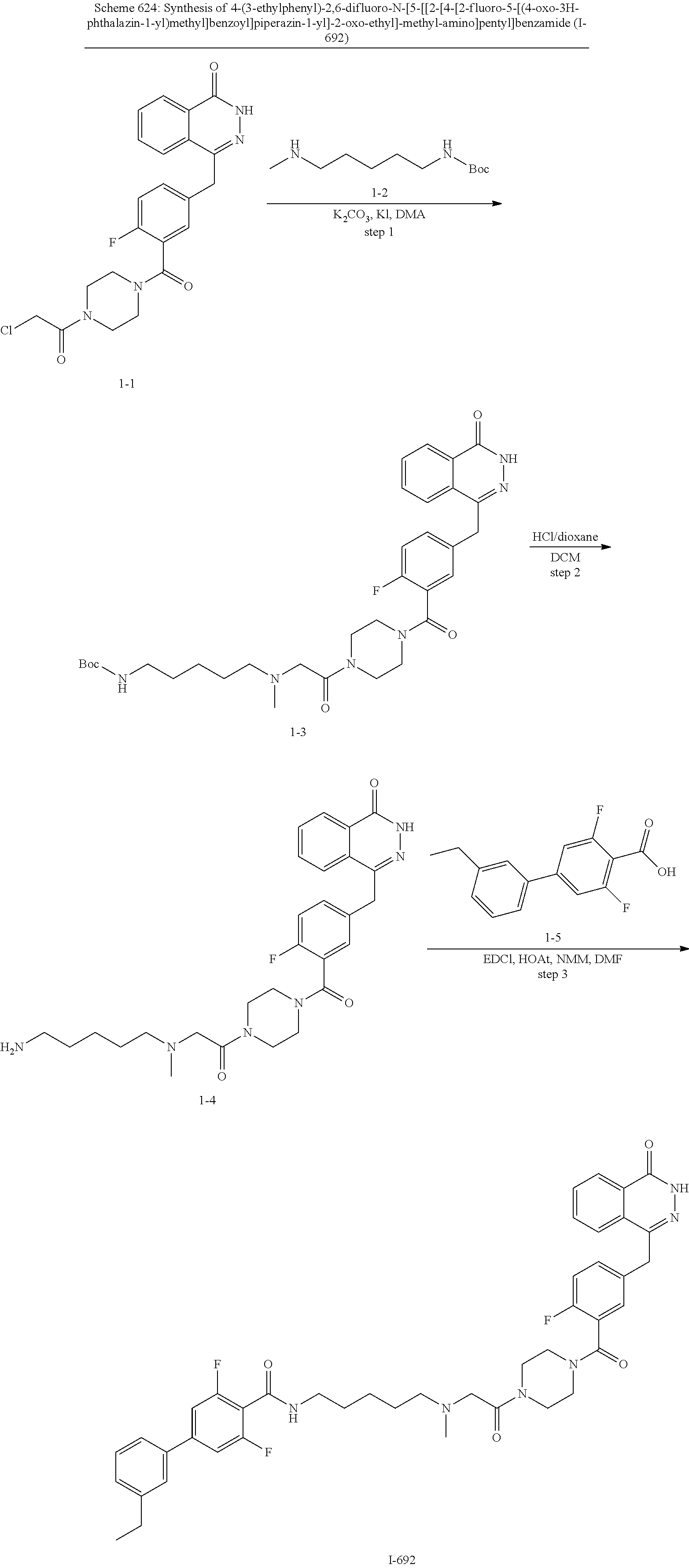

### Step 1: Synthesis of Intermediate 1-3

To a solution of intermediate 1-1 (2 g, 4.52 mmol, 1 eq) and intermediate 1-2 (976.90 mg, 4.52 mmol, 1 eq) in DMA (20 mL) was added KI (74.97 mg, 451.60 μmol, 0.1 eq) and K₂CO₃ (2.50 g, 18.06 mmol, 4 eq). The mixture was stirred at 70° C. for 1 hr. The reaction mixture was quenched by H₂O (30 mL), then extracted with EA (30 mL*2), the organic layer was dried over Na₂SO₄, filtered and concentrated under reduced pressure give a residue. The residue was purified by reverse phase chromatography (220 g of XB—C18, 20-40 μm, 120 Å) Mobile phase: A for H₂O (0.1% FA, v/v) and B for acetonitrile; Gradient: B 0%-100% in 20 min; Flow rate: 100 ml/min; Column temperature: R.T. Wavelength: 220 nm/254 nm). The eluent was concentrated to remove organic solvents and a saturated solution of NaHCO₃ (20 mL) was added and the mixture was extracted with EA (50 mL×3). The combined organic layers were dried over sodium sulfate, filtered and concentrated under reduced pressure to give product. Intermediate 1-3 (2.2 g, 3.53 mmol, 78.10% yield, 99.833% purity) was obtained as a white solid. LCMS (Method E): Rt=0.432 min, [M+H]⁺=623.4. ¹H NMR (400 MHz, CHLOROFORM-d) δ=10.73-10.50 (m, 1H), 8.54-8.36 (m, 1H), 7.88-7.64 (m, 3H), 7.41-7.29 (m, 2H), 7.08-7.04 (m, 1H), 4.57 (br s, 1H), 4.29 (s, 2H), 3.83-3.52 (m, 6H), 3.30 (d, J=16.4 Hz, 2H), 3.22-3.05 (m, 4H), 2.42-2.31 (m, 2H), 2.23 (d, J=16.8 Hz, 3H), 1.71 (s, 1H), 1.51-1.40 (m, 13H), 1.32 (d, J=6.8 Hz, 1H). ¹⁹F NMR (377 MHz, CHLOROFORM-d) δ=−117.602, −117.656.

### Step 2: Synthesis of Intermediate 1-4

To a solution of intermediate 1-3 (2.2 g, 3.53 mmol, 1 eq) in DCM (12 mL) was added HCl/dioxane (12 mL), the reaction mixture was stirred at 25° C. for 1 hr. The reaction mixture was concentrated to give a crude product. The crude product was used in the next step directly. Intermediate 1-4 (2.5 g, crude, HCl salt) was obtained as a white solid. LCMS (Method E): Rt=0.354 min, [M+H]⁺=523.3. ¹H NMR (400 MHz, DMSO-d₆) δ=12.62 (d, J=6.4 Hz, 1H), 9.78 (d, J=2.0 Hz, 1H), 8.31-8.13 (m, 2H), 8.01-7.80 (m, 2H), 7.51-7.34 (m, 1H), 7.25 (t, J=9.2 Hz, 1H), 4.95 (br s, 2H), 4.47-4.29 (m, 2H), 3.76-3.50 (m, 11H), 3.49-3.40 (m, 1H), 3.38-3.02 (m, 3H), 2.92-2.60 (m, 3H), 1.81-1.52 (m, 4H), 1.40-1.28 (m, 1H).

### Step 3: Synthesis of I-692

To a solution of intermediate 1-5 (469.08 mg, 1.79 mmol, 1 eq) in DMF (10 mL) was added EDCI (1.03 g, 5.37 mmol, 3 eq), HOAt (365.19 mg, 2.68 mmol, 375.32 μL, 1.5 eq), NMM (904.59 mg, 8.94 mmol, 983.25 μL, 5 eq), and intermediate 1-4 (1 g, 1.79 mmol, 1 eq, HCl salt), and the reaction mixture was stirred at 20° C. for 1 hr. The reaction mixture was concentrated to give crude product. The crude product was purified by reverse phase chromatography (120 g of XB—C18, 20-40 μm, 120 Å) Mobile phase: A for H₂O (0.1% NH₃·H₂O v/v) and B for acetonitrile; Gradient: B 5%-100% in 30 min; Flow rate: 100 ml/min; Column temperature: R.T. Wavelength: 220 nm/254 nm). The eluent was concentrated to remove organic solvents and lyophilized to give product. I-692 (750 mg, 971.07 μmol, 54.29% yield, 99.288% purity) was obtained as a white solid. LCMS (Method G): Rt=0.678 min, [M+H]⁺=767.5. ¹H NMR (400 MHz, DMSO-d₆) δ=12.59 (s, 1H), 8.75-8.60 (m, 1H), 8.26 (d, J=6.4 Hz, 1H), 7.99-7.93 (m, 1H), 7.91-7.79 (m, 2H), 7.62-7.48 (m, 4H), 7.46-7.35 (m, 3H), 7.32-7.17 (m, 2H), 4.31 (s, 2H), 3.74-3.45 (m, 5H), 3.38 (br s, 1H), 3.30-3.09 (m, 6H), 2.67 (t, J=7.2 Hz, 2H), 2.37-2.29 (m, 2H), 2.16 (d, J=8.8 Hz, 3H), 1.57-1.27 (m, 6H), 1.26-1.18 (m, 3H). ¹⁹F NMR (377 MHz, DMSO-d₆) δ=−113.818, −119.748.

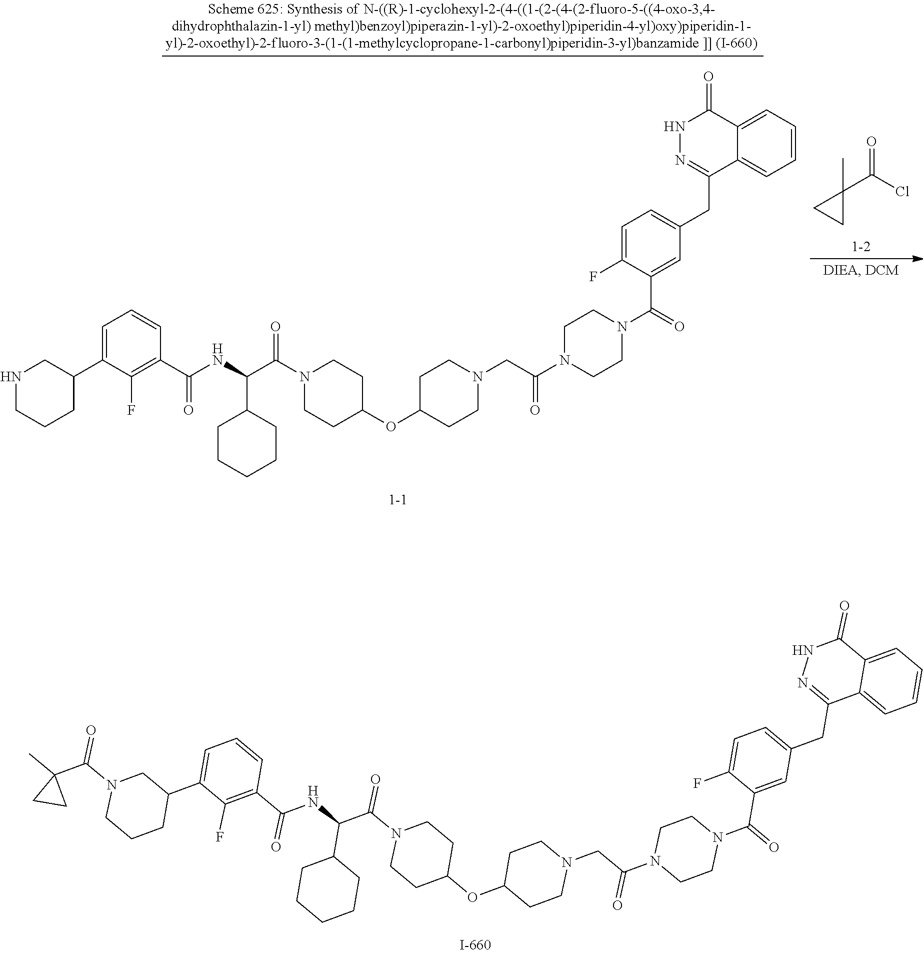

To a solution of intermediate 1-1 (700 mg, 720.48 μmol, 1 eq, HCl salt) and intermediate 1-2 (102.51 mg, 864.58 μmol, 1.2 eq) in DCM (7 mL) was added DIEA (186.23 mg, 1.44 mmol, 250.99 μL, 2 eq). The mixture was stirred at 25° C. for 1 hr. The reaction mixture was concentrated under reduced pressure to give a residue. The residue was purified by prep-HPLC (column: CD01-Phenomenex luna C18 150*25*10 um; mobile phase: [water (FA)-ACN]; gradient: 21%-51% B over 8 min), and the eluent was concentrated to remove ACN and lyophilized to give product. I-660 (405 mg, 387.48 μmol, 53.78% yield, 97.32% purity) was obtained as a white solid. LCMS (Method D): Retention time: 0.437, 0.448 min, [M+H]⁺=1017.7. SFC: Retention time: 3.226, 4.356 min. ¹H NMR (400 MHz, METHANOL-d4) δ=8.42-8.34 (m, 1H), 7.95 (d, J=6.4 Hz, 1H), 7.91-7.77 (m, 2H), 7.64-7.44 (m, 3H), 7.37 (t, J=6.8 Hz, 1H), 7.26 (t, J=7.6 Hz, 1H), 7.21-7.12 (m, 1H), 4.96 (d, J=7.6 Hz, 1H), 4.56-4.46 (m, 2H), 4.39 (s, 2H), 4.09-3.99 (m, 1H), 3.97-3.74 (m, 5H), 3.73-3.60 (m, 4H), 3.56-3.45 (m, 3H), 3.38-3.34 (m, 1H), 3.21 (d, J=2.4 Hz, 2H), 3.18-3.00 (m, 4H), 2.97-2.63 (m, 3H), 2.09-1.94 (m, 4H), 1.93-1.74 (m, 9H), 1.72-1.60 (m, 4H), 1.57-1.48 (m, 1H), 1.37-1.29 (m, 4H), 1.27-1.05 (m, 4H), 1.00-0.86 (m, 2H), 0.63 (s, 2H). ¹⁹F NMR (376 MHz, METHANOL-d4) δ=−120.77 (s), −121.62 (s).

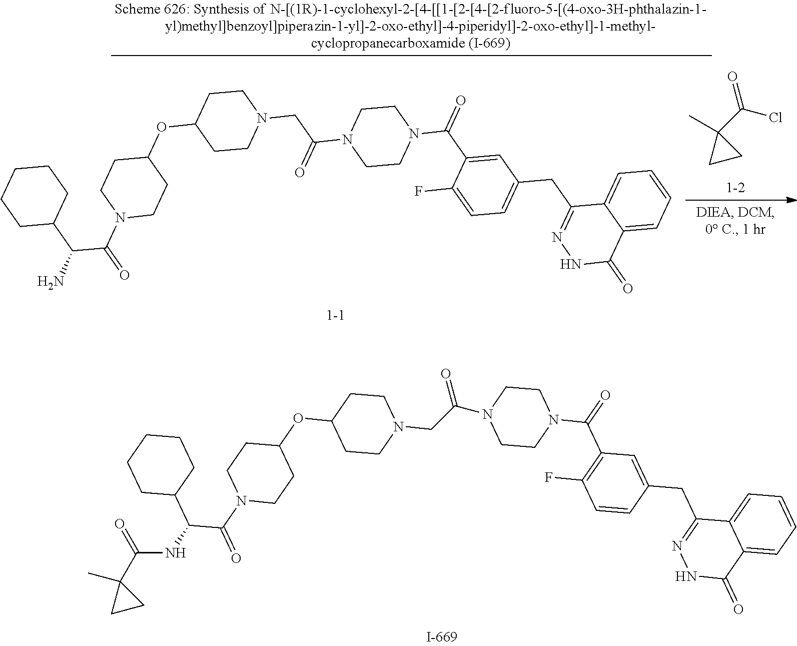

To a solution of intermediate 1-1 (350 mg, 479.53 μmol, 1 eq) in DCM (4 mL) was added DIEA (309.88 mg, 2.40 mmol, 417.63 μL, 5 eq) and intermediate 1-2 (73.91 mg, 623.39 μmol, 1.3 eq). The mixture was stirred at 0° C. for 1 hr. The reaction mixture was diluted with water (60 mL) and extracted with ethyl acetate (20 mL×3). The combined organic layers were washed with brine (20 mL×3) and dried over sodium sulfate, filtered and concentrated under reduced pressure to give a residue. The crude product was purified by reversed-phase HPLC (neutral condition) and the eluent was concentrated under reduced pressure to remove ACN and then lyophilized to give I-669 (349.97 mg, 431.01 μmol, 89.88% yield, 100% purity) as a white solid. LCMS (Method D): Rt: 0.370 min, [M+H]⁺=812.4. ¹H NMR (400 MHz, METHANOL-d4) δ=8.38 (d, J=7.6 Hz, 1H), 7.99-7.93 (m, 1H), 7.91-7.78 (m, 2H), 7.49 (d, J=2.4 Hz, 1H), 7.41-7.34 (m, 1H), 7.17 (s, 1H), 4.77 (d, J=7.6 Hz, 1H), 4.39 (s, 2H), 4.04-3.70 (m, 6H), 3.67 (s, 1H), 3.60-3.35 (m, 5H), 3.28-3.12 (m, 3H), 2.76 (d, J=1.2 Hz, 2H), 2.36-2.15 (m, 2H), 1.94-1.43 (m, 15H), 1.41-1.33 (m, 3H), 1.32-1.15 (m, 3H), 1.14-0.94 (m, 4H), 0.68-0.58 (m, 2H). ¹⁹F NMR (376 MHz, METHANOL-d4) δ=−120.794. SFC: Rt: 0.797 min.

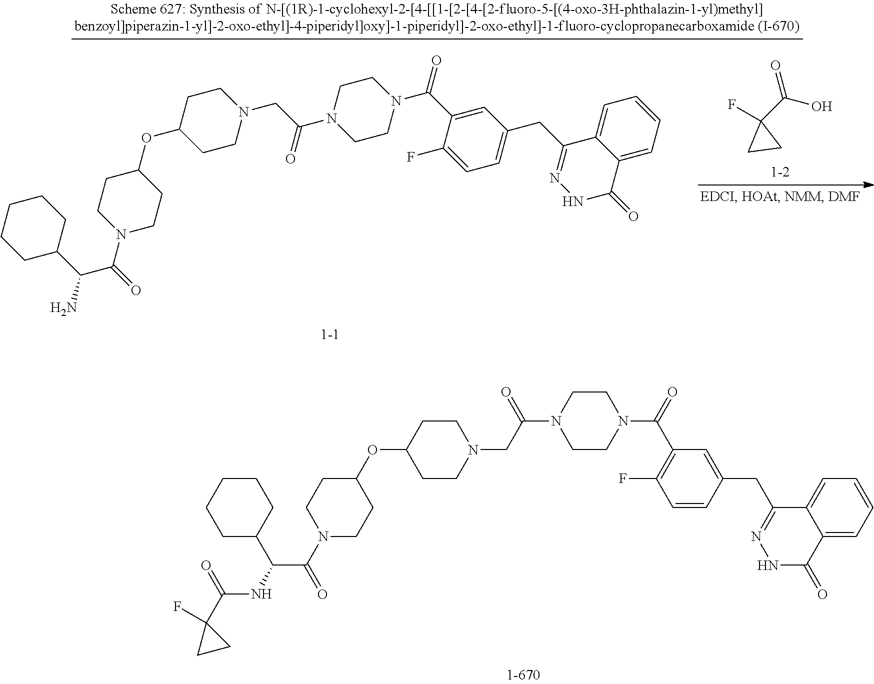 [SMILES: N[C@@H](C(=O)N1CCC(OC2CCN(CC(=O)N3CCN(C(=O)c4cc(Cc5n[nH]c(=O)c6ccccc56)ccc4F)CC3)CC2)CC1)C1CCCCC1 ; O=C(O)C1(F)CC1 ; O=C(CN1CCC(OC2CCN(C(=O)[C@H](NC(=O)C3(F)CC3)C3CCCCC3)CC2)CC1)N1CCN(C(=O)c2cc(Cc3n[nH]c(=O)c4ccccc34)ccc2F)CC1]

To a solution of intermediate 1-2 (108.65 mg, 1.04 mmol, 1 eq) in DMF (8 mL) was added HOAt (142.09 mg, 1.04 mmol, 146.03 μL, 1 eq), EDCI (600.36 mg, 3.13 mmol, 3 eq), NMM (527.95 mg, 5.22 mmol, 573.86 μL, 5 eq) and intermediate 1-1 (800 mg, 1.04 mmol, 1 eq, HCl salt). The mixture was stirred at 25° C. for 1 hr. The residue was diluted with H₂O (20 mL) and extracted with EA (15 mL*3), dried over Na₂SO₄, filtered and concentrated under reduced pressure to give a residue. The crude product was purified by reversed phase chromatography (0.1% FA condition), which was concentrated and lyophilized to afford I-670 (280.31 mg, 321.13 μmol, 30.76% yield, 98.7500% purity, FA salt) as a white solid. LCMS (Method D): Rt=0.373 min, [M+H]⁺=816.2. ¹H NMR (400 MHz, METHANOL-d4) δ=8.45 (s, 1H), 8.37 (d, J=7.6 Hz, 1H), 8.01-7.92 (m, 1H), 7.91-7.78 (m, 2H), 7.50 (s, 1H), 7.37 (t, J=6.0 Hz, 1H), 7.17 (t, J=9.2 Hz, 1H), 4.82 (d, J=7.6 Hz, 2H), 4.39 (s, 2H), 4.04-3.90 (m, 1H), 3.88-3.74 (m, 4H), 3.74-3.57 (m, 5H), 3.56-3.39 (m, 4H), 3.38-3.34 (m, 1H), 3.24 (s, 1H), 3.13-2.94 (m, 2H), 2.85-2.57 (m, 2H), 1.97 (d, J=4.4 Hz, 2H), 1.78 (d, J=10.4 Hz, 6H), 1.70-1.64 (m, 2H), 1.61-1.44 (m, 2H), 1.39-1.16 (m, 7H), 1.13-0.97 (m, 2H). ¹⁹F NMR (376 MHz, METHANOL-d4) δ=−120.71 (s), −199.63 (s). SFC: Rt=1.432 min.

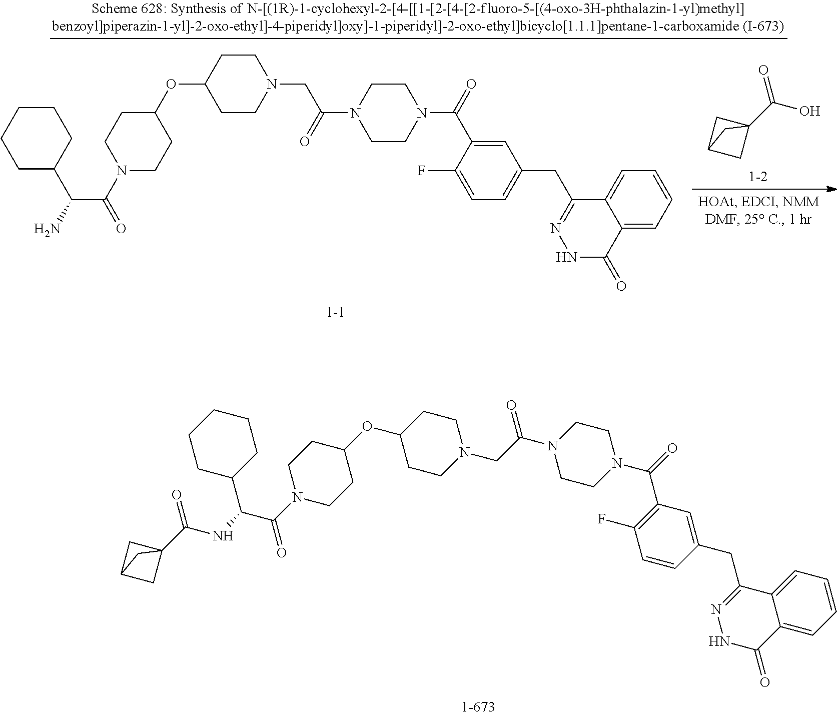 [SMILES: N[C@@H](C(=O)N1CCC(OC2CCN(CC(=O)N3CCN(C(=O)c4cc(Cc5n[nH]c(=O)c6ccccc56)ccc4F)CC3)CC2)CC1)C1CCCCC1 ; O=C(O)C12CC(C1)C2 ; O=C(CN1CCC(OC2CCN(C(=O)[C@H](NC(=O)C34CC(C3)C4)C3CCCCC3)CC2)CC1)N1CCN(C(=O)c2cc(Cc3n[nH]c(=O)c4ccccc34)ccc2F)CC1]

To a solution of intermediate 1-2 (117.05 mg, 1.04 mmol, 1 eq) in DMF (8 mL) was added EDCI (600.36 mg, 3.13 mmol, 3 eq), HOAt (142.09 mg, 1.04 mmol, 146.03 μL, 1 eq) and NMM (527.95 mg, 5.22 mmol, 573.86 μL, 5 eq). Then intermediate 1-1 (800 mg, 1.04 mmol, 1 eq, HCl salt) was added into the mixture. The mixture was stirred at 25° C. for 1 hr. The mixture was washed with water (10 mL) and extracted with EA (15 mL*3). The combined organic layers were dried over sodium sulfate, filtered and concentrated under reduced pressure to give a residue. The residue was purified by prep-HPLC (column: CD05-Phenomenex luna C18 150*40*10 um; mobile phase: [water(FA)-ACN]; gradient: 5%-35% B over 10 min) and the eluent was concentrated under reduced pressure to remove ACN and lyophilized to give product. I-673 (391.50 mg, 448.24 μmol, 42.94% yield, 99.61% purity, FA salt) was obtained as a white solid. LCMS (Method D): Rt=0.385 min, [M+H]⁺=824.4. SFC: Rt=1.374 min. ¹H NMR (400 MHz, METHANOL-d4) δ=8.44 (d, J=1.2 Hz, 1H), 8.37 (d, J=7.6 Hz, 1H), 7.94 (d, J=4.4 Hz, 1H), 7.90-7.80 (m, 2H), 7.62-7.60 (m, 1H), 7.50 (s, 1H), 7.37-7.35 (m, 1H), 7.17-7.16 (m, 1H), 4.68 (d, J=8.4 Hz, 1H), 4.38 (s, 2H), 3.92 (s, 2H), 3.85-3.68 (m, 7H), 3.62-3.49 (m, 3H), 3.44 (s, 2H), 3.36 (s, 1H), 3.27-3.13 (m, 3H), 2.94 (d, J=2.0 Hz, 2H), 2.43 (s, 1H), 2.06 (s, 6H), 2.02 (d, J=9.2 Hz, 2H), 1.84 (d, J=16.0 Hz, 4H), 1.75 (d, J=9.6 Hz, 4H), 1.70-1.58 (m, 3H), 1.53-1.45 (m, 1H), 1.30-1.15 (m, 3H), 0.99-0.98 (m, 2H). ¹⁹F NMR (376 MHz, MeOD-d4) δ=−120.700.

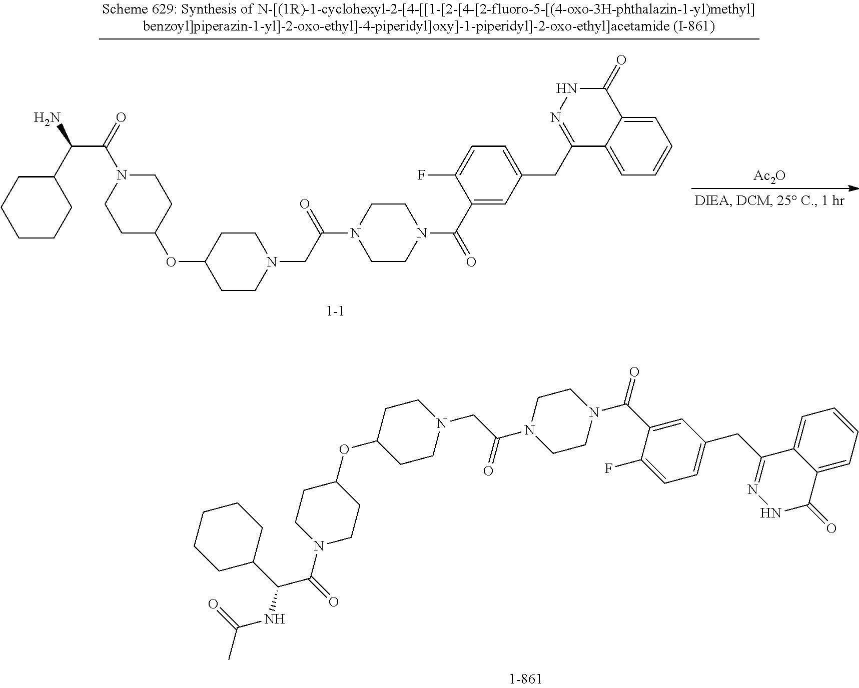 [SMILES: "Ac2O" ; N[C@@H](C(=O)N1CCC(OC2CCN(CC(=O)N3CCN(C(=O)c4cc(Cc5n[nH]c(=O)c6ccccc56)ccc4F)CC3)CC2)CC1)C1CCCCC1 ; CC(=O)N[C@@H](C(=O)N1CCC(OC2CCN(CC(=O)N3CCN(C(=O)c4cc(Cc5n[nH]c(=O)c6ccccc56)ccc4F)CC3)CC2)CC1)C1CCCCC1]

To a solution of intermediate 1-1 (800 mg, 1.04 mmol, 1 eq, HCl salt) in DCM (8 mL) was added DIEA (404.75 mg, 3.13 mmol, 545.48 μL, 3 eq) and Ac₂O (106.57 mg, 1.04 mmol, 98.04 μL, 1 eq). The mixture was stirred at 25° C. for 1 hr. The mixture was concentrated in vacuum. The crude product was purified by prep-HPLC (column: CD05-Phenomenex luna C18 150*40*10 um; mobile phase: [water (FA)-ACN]; gradient: 3%-33% B over 10 min). The eluent was concentrated under reduced pressure to remove ACN and then lyophilized to give I-861 (440.71 mg, 536.81 μmol, 51.42% yield, 99.63% purity, FA salt) as a white solid. LCMS (Method D): Retention time: 0.341 min, [M+H]⁺=772.4. SFC: Retention time: 1.331 min. ¹H NMR (400 MHz, METHANOL-d₄) δ=8.37 (d, J=7.2 Hz, 1H), 7.99-7.92 (m, 1H), 7.91-7.81 (m, 2H), 7.78-7.74 (m, 1H), 7.73-7.68 (m, 1H), 7.51-7.36 (m, 5H), 7.20-7.13 (m, 1H), 7.00-6.88 (m, 2H), 4.73-4.50 (m, 3H), 4.39 (s, 2H), 3.92-3.86 (m, 1H), 3.85-3.78 (m, 4H), 3.76-3.70 (m, 2H), 3.67-3.62 (m, 1H), 3.59-3.41 (m, 6H), 3.28-3.25 (m, 2H), 3.21-3.10 (m, 3H), 2.83-2.63 (m, 4H), 2.40-2.25 (m, 2H), 2.07-2.01 (m, 1H), 1.91-1.76 (m, 10H), 1.73-1.50 (m, 8H), 1.35-1.18 (m, 6H), 1.15-1.02 (m, 2H). ¹⁹FNMR (376 MHz, MeOD-d₆) δ=−120.757.

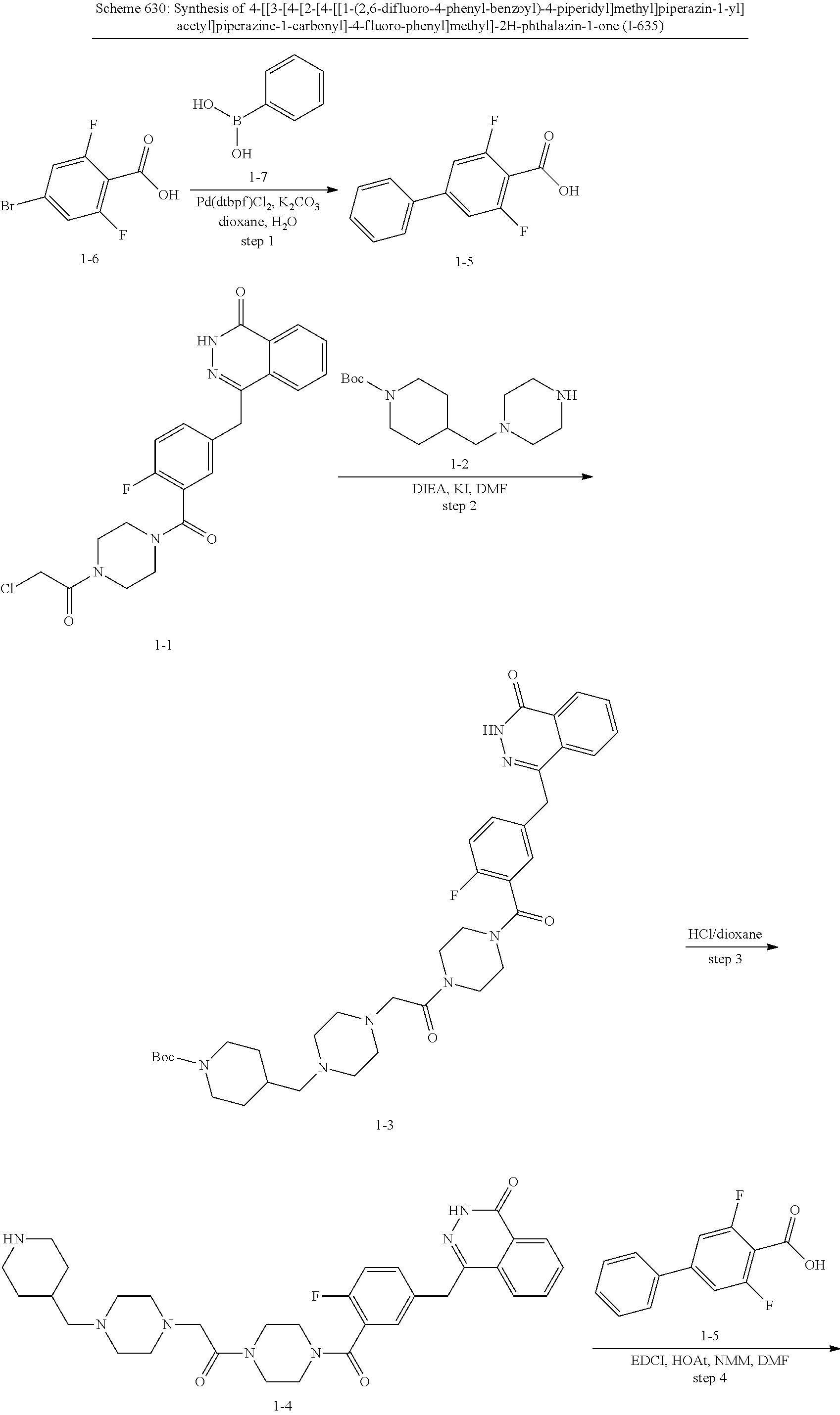

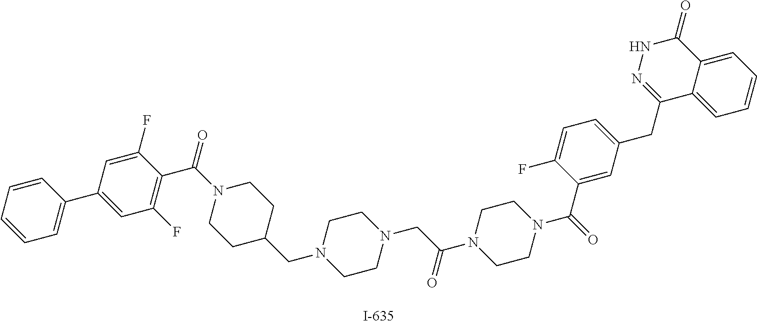 [SMILES: O=C(CN1CCN(CC2CCN(C(=O)c3c(F)cc(-c4ccccc4)cc3F)CC2)CC1)N1CCN(C(=O)c2cc(Cc3n[nH]c(=O)c4ccccc34)ccc2F)CC1]

### Step 1: Synthesis of Intermediate 1-5

A mixture of Intermediate 1-6 (2 g, 8.44 mmol, 1 eq), Intermediate 1-7 (1.13 g, 9.28 mmol, 1.1 eq), Pd(dppf)Cl₂ (617.48 mg, 843.89 μmol, 0.1 eq) and K₂CO₃ (2.33 g, 16.88 mmol, 2 eq) in dioxane (16 mL) and H₂O (4 mL) was degassed and purged with N₂ 3 times, and then the mixture was stirred at 100° C. for 2 hr under N₂ atmosphere. The mixture was diluted with H₂O (10 mL) and extracted with EtOAc (20 mL*3). The aqueous phase was adjusted to pH=3 with 3M HCl solution and extracted with EtOAc (20 mL*3). The organic layer was concentrated under vacuum Intermediate 1-5 (1.79 g, 7.64 mmol, 90.57% yield) was obtained as a brown solid. ¹H NMR (400 MHz, DMSO-d₆) δ=14.73-12.94 (m, 1H), 7.77 (br d, J=7.0 Hz, 2H), 7.55 (br d, J=9.6 Hz, 2H), 7.51-7.42 (m, 3H). ¹⁹F NMR (377 MHz, DMSO-d₆) δ=−111.303.

### Step 2: Synthesis of Intermediate 1-3

To a solution of Intermediate 1-1 (5 g, 11.29 mmol, 1 eq) in DMF (50 mL) was added Intermediate 1-2 (3.20 g, 11.29 mmol, 1 eq), DIEA (5.84 g, 45.16 mmol, 7.87 mL, 4 eq) and KI (7.50 g, 45.16 mmol, 4 eq). The mixture was stirred at 25° C. for 1 hr. The mixture was diluted with water (20 mL) and extracted with EtOAc (40 mL*3). The organic layers were washed with brine (10 mL*3) and dried with anhydrous Na₂SO₄, then filtered and concentrated under vacuum. The crude product was purified by reversed-phase chromatography (0.1% NH₃H₂O condition) and concentrated under vacuum to remove MeCN and dried by lyophilization. Intermediate 1-3 (3.9 g, 5.65 mmol, 50.08% yield) as a yellow solid was obtained. LCMS (Method G): Rt=0.593 min, [M+H]⁺=690.5. ¹H NMR (400 MHz, DMSO-d₆) δ=12.60 (s, 1H), 8.26 (br d, J=7.6 Hz, 1H), 7.99-7.93 (m, 1H), 7.88 (br t, J=7.4 Hz, 1H), 7.84-7.79 (m, 1H), 7.47-7.35 (m, 2H), 7.22 (br t, J=8.8 Hz, 1H), 4.33 (s, 2H), 3.90 (br d, J=10.4 Hz, 2H), 3.63 (br d, J=9.0 Hz, 3H), 3.49 (br d, J=17.4 Hz, 2H), 3.36 (br d, J=15.0 Hz, 2H), 3.21-3.06 (m, 4H), 2.66 (br s, 2H), 2.37 (br s, 7H), 2.06 (br dd, J=6.2, 15.6 Hz, 2H), 2.00-1.95 (m, 1H), 1.61 (br d, J=5.5 Hz, 4H), 1.37 (s, 9H). ¹⁹F NMR (377 MHz, DMSO-d₆) δ=−113.725, −119.736.

### Step 3: Synthesis of Intermediate 1-4

To a solution of Intermediate 1-3 (3.9 g, 5.65 mmol, 1 eq) in DCM (13 mL) was added HCl/dioxane (2 M, 26 mL, 9.20 eq). The mixture was stirred at 25° C. for 0.5 hr. The mixture was concentrated under vacuum. The compoud was used in the next step without purification. Intermediate 1-4 (4 g, crude) as white solid was obtained. LCMS (Method E): Rt=0.355 min, [M+H]⁺=590.4.

### Step 4: Synthesis of I-635

To a solution of Intermediate 1-4 (125.90 mg, 213.49 μmol, 1 eq) in DMF (1 mL) was added Intermediate 1-5 (50.00 mg, 213.49 μmol, 1 eq), EDCI (81.85 mg, 426.99 μmol, 2 eq), NMM (107.97 mg, 1.07 mmol, 117.36 μL, 5 eq) and HOAt (29.06 mg, 213.49 μmol, 29.87 μL, 1 eq). The mixture was stirred at 25° C. for 1 hr. The mixture was diluted with water (5 ml) and extracted with EtOAc (10 ml*3). The organic layer was concentrated under vacuum. The residue was purified by Prep-HPLC (column: Waters Xbridge 150*25 mm*5 um; mobile phase: [water (ammonia hydroxide v/v)-ACN]; gradient: 28%-58% B over 10 min) and dried by lyophilization. I-635 (10 mg, 12.41 μmol, 5.81% yield, 100% purity) was obtained as a white solid. LCMS (Method G): Rt=0.636 min, [M+H]⁺=806.4. SFC: Rt=1.970 min. ¹H NMR (400 MHz, METHANOL-d₄) δ=8.38-8.34 (m, 1H), 7.97-7.92 (m, 1H), 7.90-7.79 (m, 2H), 7.69-7.64 (m, 2H), 7.51-7.45 (m, 3H), 7.45-7.41 (m, 1H), 7.40-7.36 (m, 3H), 7.18-7.14 (m, 1H), 4.68 (br d, J=12.8 Hz, 1H), 4.38 (s, 2H), 3.84-3.65 (m, 4H), 3.63-3.49 (m, 3H), 3.33 (br s, 1H), 3.29-3.12 (m, 4H), 2.95-2.87 (m, 1H), 2.51 (br s, 8H), 2.26-2.21 (m, 2H), 1.95-1.76 (m, 3H), 1.24-1.08 (m, 2H). ¹⁹F NMR (377 MHz, METHANOL-d₄) δ=−115.201, −120.690.

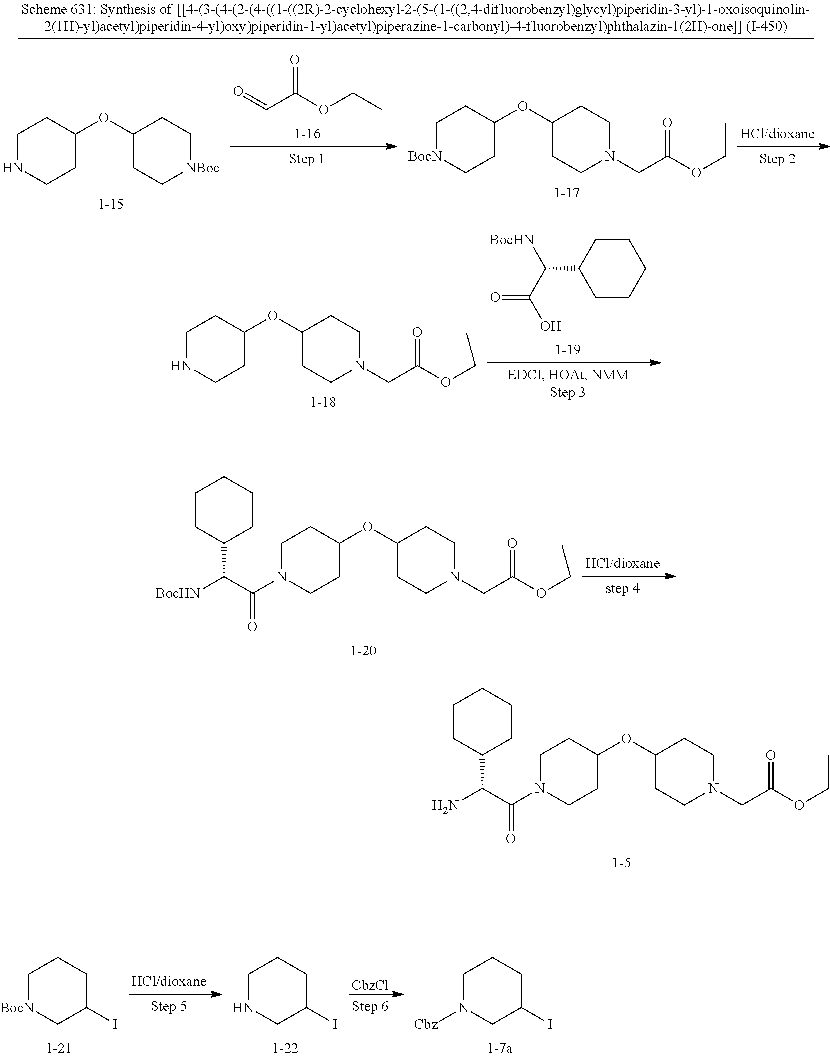

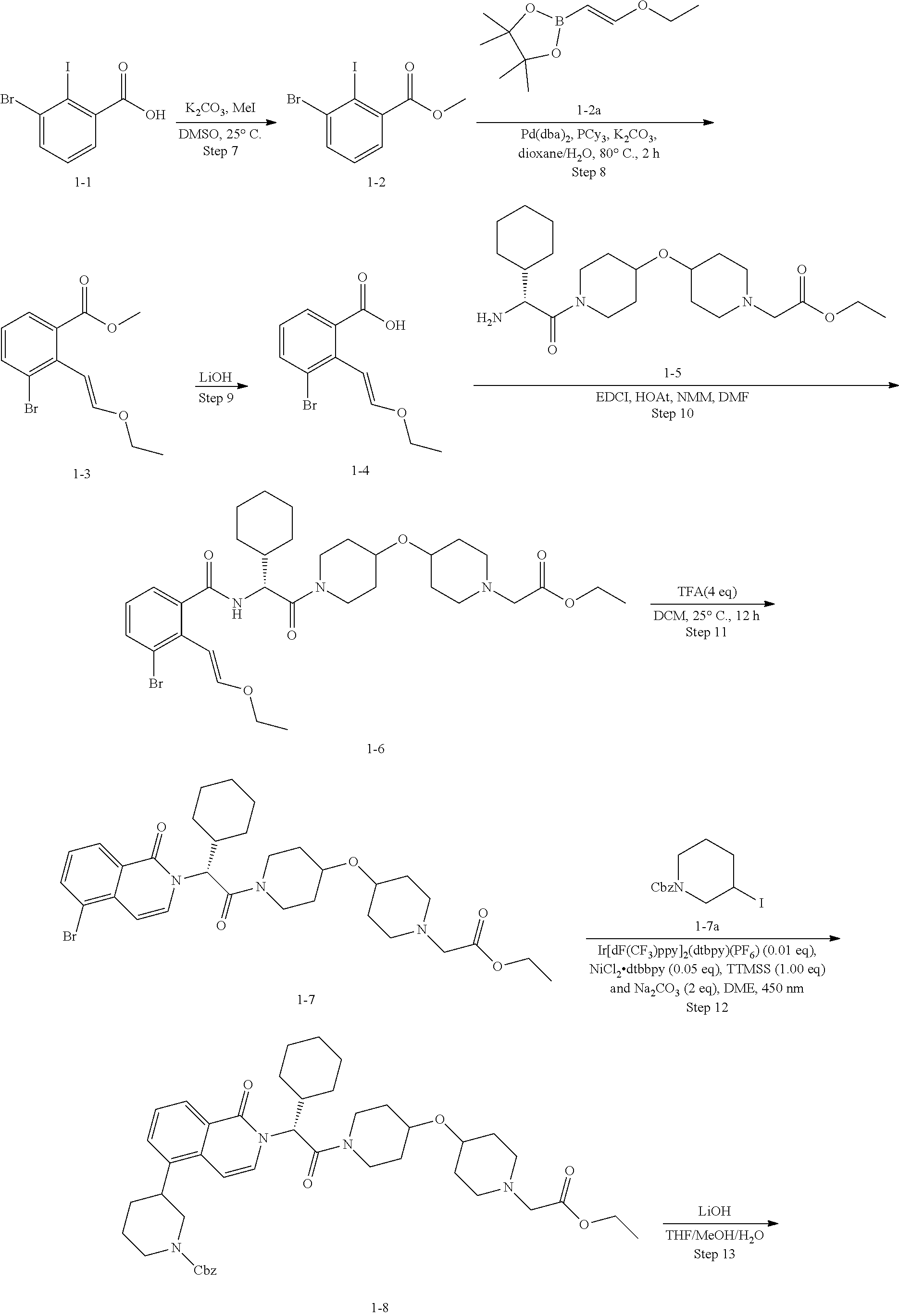

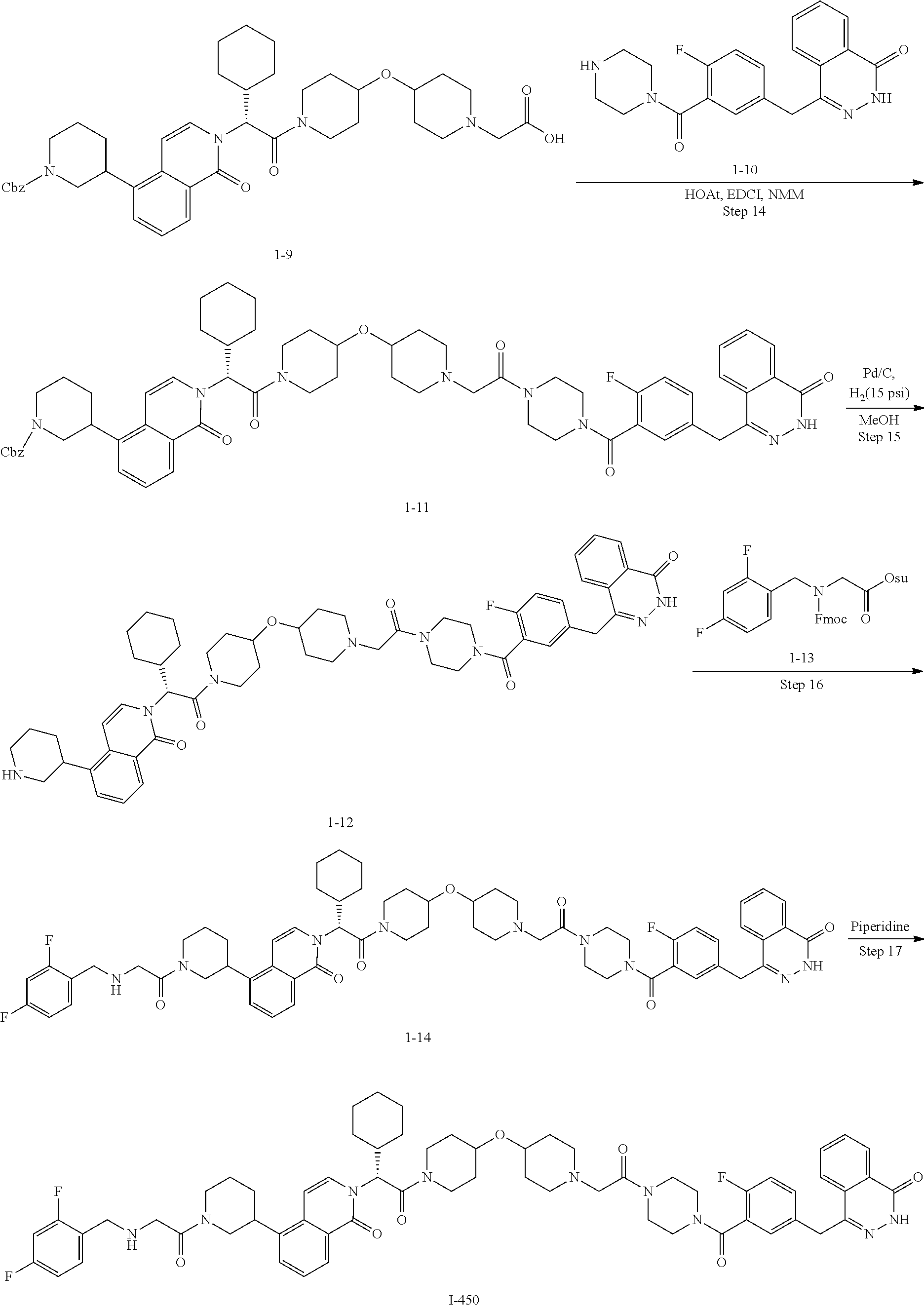

### Step 1: Synthesis of Intermediate 1-17

A solution of Intermediate 1-15 (10 g, 35.16 mmol, 1 eq) and Intermediate 1-16 (10.77 g, 52.74 mmol, 1.5 eq) in DCM (100 mL) was stirred at 25° C. for 0.5 h. Then NaBH(OAc)₃ (18.63 g, 87.91 mmol, 2.5 eq) was added to the mixture and the mixture was stirred at 25° C. for 1.5 h. The reaction mixture was quenched by H₂O (50 mL), and then adjusted to pH=8 with NH₃·H₂O and extracted with CHCl₃:i-PrOH (60 mL*3). The combined organic layers were dried over anhydrous Na₂SO₄, filtered and concentrated under reduced pressure to give Intermediate 1-17 (13 g, crude) as a brown oil. LCMS (Method E): Rt=0.538 min, [M+H]⁺=371.3.

### Step 2: Synthesis of Intermediate 1-18

To a solution of Intermediate 1-17 (13 g, 35.09 mmol, 1 eq) in DCM (20 mL) was added HCl/dioxane (2 M, 50 mL, 2.85 eq) and the mixture was stirred at 25° C. for 1.5 hr. The mixture was concentrated under vacuum to give Intermediate 1-18 (11 g, crude, HCl salt) as a red oil. LCMS (Method G): Rt=0.590 min, [M+H]⁺=271.3.

### Step 3: Synthesis of Intermediate 1-20

To a solution of Intermediate 1-18 (11 g, 35.85 mmol, 1 eq, HCl salt), Intermediate 1-19 (9.23 g, 35.85 mmol, 1 eq) and HOAt (4.88 g, 35.85 mmol, 5.02 mL, 1 eq) in DMF (50 mL) was added EDCI (13.75 g, 71.70 mmol, 2 eq) and NMM (18.13 g, 179.25 mmol, 19.71 mL, 5 eq). The mixture was stirred at 25° C. for 1 hr. The mixture was diluted with water (10 mL), extracted with EtOAc (60 mL*2), and washed with brine (30*3). The organic layer was concentrated under vacuum. The residue was purified by reversed-phase chromatography (0.1% NH₃·H₂O condition) and concentrated under vacuum to remove MeCN. The aqueous phase was extracted with EA (50 mL*3), the organic phase was dried with anhydrous Na₂SO₄, filtered and concentrated under vacuum. The residue was purified by flash silica gel chromatography (ISCO; 120 g SepaFlash Silica Flash Column, Eluent of 0˜10% DCM (1% NH₃·H₂O)/MeOH @100 mL/min). The residue was further purified by prep-HPLC (column: Kromasil Eternity XT 250*80 mm*10 um; mobile phase: [water (NH₄HCO₃)-ACN]; gradient: 40%-70% B over 30 min) and concentrated under vacuum to remove MeCN. The aqueous phase was extracted with EA (100 mL*3), the organic phase was dried with anhydrous Na₂SO₄, filtered and concentrated under vacuum. Intermediate 1-20 (6.25 g, 12.26 mmol, 34.20% yield) as yellow oil was obtained. LCMS (Method G): Rt=0.660 min, [M+H]⁺=510.4.

### Step 4: Synthesis of Intermediate 1-5

To a solution of Intermediate 1-20 (2.2 g, 4.32 mmol, 1 eq) in DCM (10 mL) was added HCl/dioxane (4 M, 10 mL, 9.27 eq). The mixture was stirred at 25° C. for 2 h. The mixture was concentrated under vacuum to give a crude product. Intermediate 1-5 (2.1 g, crude, HCl salt) was obtained as a yellow solid. LCMS (Method G): Rt=0.543 min, [M+H]+=410.3.

### Step 5: Synthesis of Intermediate 1-22

To a solution of Intermediate 1-21 (5 g, 16.07 mmol, 1 eq) in DCM (20 mL) was added HCl/dioxane (2 M, 40.00 mL, 4.98 eq) and the mixture was stirred at 25° C. for 12 hr. The mixture was concentrated under vacuum. Intermediate 1-22 (4 g, crude, HCl salt) as a white solid was obtained.

### Step 6: Synthesis of Intermediate 1-7a

To a solution of Intermediate 1-22 (4 g, 16.16 mmol, 1 eq, HCl salt) and CbzCl (4.14 g, 24.24 mmol, 3.46 mL, 1.5 eq) in THF (20 mL) and H₂O (20 mL) was added K₂CO₃ (2.23 g, 16.16 mmol, 1 eq). The mixture was stirred at 25° C. for 2 hr. The mixture was diluted with water (10 mL), and extracted with EtOAc (10 mL*3). The organic layer was dried with anhydrous Na₂SO₄, filtered and concentrated under vacuum. The residue was purified by flash silica gel chromatography (ISCO; 80 g SepaFlash Silica Flash Column, Eluent of 0˜30% Ethylacetate/Petroleum ether gradient @100 mL/min). Intermediate 1-7a (5.2 g, 15.06 mmol, 93.22% yield) as colorless oil was obtained. ¹H NMR (400 MHz, CHLOROFORM-d) δ=7.41-7.30 (m, 5H), 5.23-5.05 (m, 2H), 4.35-4.06 (m, 2H), 3.97-3.80 (m, 1H), 3.58-3.28 (m, 1H), 3.16 (br d, J=5.9 Hz, 1H), 2.29 (br s, 1H), 2.11-1.92 (m, 1H), 1.84-1.69 (m, 1H), 1.68-1.57 (m, 1H).

### Step 7: Synthesis of Intermediate 1-2

To a solution of Intermediate 1-1 (5 g, 15.29 mmol, 1 eq) and K₂CO₃ (10.57 g, 76.47 mmol, 5 eq) in DMSO (50 mL) was added MeI (3.26 g, 22.94 mmol, 1.43 mL, 1.5 eq) dropwise at 25° C. under N₂. The mixture was stirred at 25° C. for 1 hr. The mixture was diluted with water (10 mL), extracted with EtOAc (50 mL*3) and washed with brine (15 mL*3). The organic layer was concentrated under vacuum. The residue was purified by flash silica gel chromatography (ISCO; 220 g Sepa Flash Silica Flash Column, Eluent of 0˜5% Ethyl acetate/Petroleum ether gradient @100 mL/min). Intermediate 1-3 (5.9 g, crude) (˜60% purity by NMR) as a yellow oil was obtained. LCMS (Method G): Rt=0647 min, [M+H]⁺=285.2. ¹H NMR (400 MHz, CHLOROFORM-d) δ=7.68 (dd, J=1.3, 8.0 Hz, 1H), 7.54 (dd, J=1.1, 7.8 Hz, 1H), 7.14-7.05 (m, 1H), 6.54 (d, J=13.0 Hz, 1H), 5.99 (d, J=13.1 Hz, 1H), 3.95 (q, J=7.0 Hz, 2H), 3.87-3.85 (m, 3H), 1.30 (t, J=7.1 Hz, 3H).

### Step 9: Synthesis of Intermediate 1-4

To a solution of Intermediate 1-3 (4.9 g, 17.18 mmol, 1 eq) in H₂O (20 mL), THF (20 mL) and MeOH (10 mL) was added LiOH·H₂O (2.88 g, 68.74 mmol, 4 eq). The mixture was stirred at 25° C. for 4 h. The mixture was concentrated under vacuum to remove THF and MeOH. Then the aqueous layer was adjusted to pH=3 by citric acid and extracted with EA (10 ml*3). The combined organic layers was washed with brine (10 ml*3), dried over anhydrous Na₂SO₄, filtered and concentrated under vacuum to give Intermediate 1-4 (3.4 g, crude) as a yellow oil.

### Step 10: Synthesis of Intermediate 1-6

To a solution Intermediate 1-4 (3.4 g, 12.54 mmol, 1 eq) in DMF (40 mL) was added EDCI (4.81 g, 25.08 mmol, 2 eq), HOAt (1.71 g, 12.54 mmol, 1.75 mL, 1 eq), NMM (6.34 g, 62.71 mmol, 6.89 mL, 5 eq), HOAt (1.71 g, 12.54 mmol, 1.75 mL, 1 eq) and Intermediate 1-5 (3.92 g, 8.78 mmol, 0.7 eq, HCl salt). The mixture was stirred at 25° C. for 0.5 h. The reaction mixture was quenched by H₂O (100 mL) and extracted with EA (50 mL*3). The combined organic layers were washed with brine (50 mL*3), dried over anhydrous Na₂SO₄, filtered and concentrated under reduced pressure to give a residue. The crude product was purified by reversed-phase HPLC (0.1% NH₃·H₂O). Intermediate 1-6 (3.05 g, 4.56 mmol, 36.40% yield, 99.177% purity) was obtained as a yellow oil. LCMS (Method G): Rt=0.753 min, [M+H]⁺=664.3.

### Step 11: Synthesis of Intermediate 1-7

To a mixture of Intermediate 1-6 (3.05 g, 4.60 mmol, 1 eq) in DCM (30 mL) was added TFA (1.57 g, 13.81 mmol, 1.03 mL, 3 eq), then the mixture was stirred at 25° C. for 12 hr. The mixture was adjusted to pH=8 with aq. Na₂CO₃ and extracted with DCM (15 mL*2). The organic layer was concentrated under vacuum. Intermediate 1-7 (2.85 g, crude) as a brown oil was obtained. LCMS (Method G): Rt=0.806 min, [M+H]⁺=618.3.

### Step 12: Synthesis of Intermediate 1-8

A mixture of Intermediate 1-7 (0.4 g, 648.73 μmol, 1 eq), Intermediate 1-7a (291.11 mg, 843.36 μmol, 1.3 eq), TTMSS (161.31 mg, 648.73 μmol, 200.14 μL, 1 eq), Ir[dF(CF₃)ppy]₂(dtbpy)(PF₆) (7.28 mg, 6.49 μmol, 0.01 eq), Na₂CO₃ (206.28 mg, 1.95 mmol, 3 eq) and 4-tert-butyl-2-(4-tert-butyl-2-pyridyl)pyridine;dichloronickel (1.29 mg, 3.24 μmol, 0.005 eq) in DME (5 mL) was degassed and purged with N₂, and then the mixture was stirred at 25° C. for 16 hr and irradiated with a 455 nm blue LED. The mixture was diluted with water (5 mL) and extracted with EtOAc (5 mL*3). The organic layer was concentrated under vacuum. The residue was purified by prep-HPLC (column: Waters Xbridge Prep OBD C18 150*40 mm*10 um; mobile phase: [water(NH4HCO3)-ACN]; gradient: 62%-92% B over 15 min) and concentrated under vacuum. Intermediate 1-8 (240 mg, 317.90 μmol, 49.00% yield) as a yellow solid was obtained. LCMS (Method G): Rt=0.812, 0.827 min, [M+H]⁺=755.6. SFC: Rt=1.671, 2.090 min.

### Step 13: Synthesis of Intermediate 1-9

To a solution of Intermediate 1-8 (240 mg, 317.90 μmol, 1 eq) in THF (1 mL), H₂O (0.5 mL) and MeOH (0.5 mL) was added LiOH·H₂O (26.68 mg, 635.80 μmol, 2 eq). The mixture was stirred at 25° C. for 1 hr. The mixture was concentrated under vacuum and adjusted to pH=5 with 2N HCl and extracted with EtOAc (10 mL*3). The organic layer was dried with anhydrous Na₂SO₄, filtered and concentrated under vacuum. Intermediate 1-9 (200 mg, crude) as a yellow solid was obtained. LCMS (Method G): Rt=0.552, 0.572 min, [M+H]⁺=727.5.

### Step 14: Synthesis of Intermediate 1-11

To a solution of Intermediate 1-9 (0.2 g, 275.14 μmol, 1 eq), Intermediate 1-10 (100.81 mg, 275.14 μmol, 1 eq) and HOAt (37.45 mg, 275.14 μmol, 38.49 μL, 1 eq) in DMF (2 mL) was added EDCI (105.49 mg, 550.28 μmol, 2 eq) and NMM (139.15 mg, 1.38 mmol, 151.25 μL, 5 eq). The mixture was stirred at 25° C. for 2 hrs. The mixture was poured into water (5 mL) and filtered. The filter cake was dried under vacuum. Intermediate 1-11 (350 mg, crude) as a yellow solid was obtained. LCMS (Method): Rt=2.249, 2.301 min, [M+H]⁺=1075.8. SFC: Rt=3.784, 4.584 min.

### Step 15: Synthesis of Intermediate 1-12

To a solution of Pd(OH)₂/C (100 mg, 142.41 μmol, 20% purity, 1.53 eq) in DMF (2 mL) was added Intermediate 1-11 (0.1 g, 93.00 μmol, 1 eq). The suspension was degassed and purged with H₂ 3 times. The mixture was stirred under H₂ (50 psi) at 60° C. for 12 hr. The mixture was filtered through a pad of Celite and washed with DMF (1 mL*4). Intermediate 1-12 (88 mg, crude) as a yellow liquid in DMF was obtained. LCMS (Method G): Rt=0.854, 0.946 min, [M+H]⁺=941.7.

### Step 16: Synthesis of Intermediate 1-14

To the solution of Intermediate 1-12 (88 mg, 93.50 μmol, 1 eq) in DMF (5 mL) was added Intermediate 1-13 (73.00 mg, 140.26 μmol, 1.5 eq) and DIEA (36.25 mg, 280.51 μmol, 48.86 μL, 3 eq), and the mixture was stirred at 25° C. for 2 hr. The mixture was used in the next step without work up. Intermediate 1-14 (125 mg, 92.83 μmol, 99.28% yield) as a brown liquid in DMF was obtained. LCMS (Method G): Rt=0.859, 0.888 min, [M+H]⁺=1347.7.

### Step 17: Synthesis of I-450

A mixture of Intermediate 1-14 (125 mg, 92.83 μmol, 1 eq) and piperidine (862.20 mg, 10.13 mmol, 1 mL, 109.08 eq) was stirred at 25° C. for 0.25 hr. The mixture was diluted with water (2 mL) and extracted with EtOAc (2 mL*3). The organic layer was concentrated under vacuum. The residue was purified by prep-HPLC (column: Waters Xbridge 150*25 mm*5 um; mobile phase: [water(NH₄HCO₃)-ACN]; gradient: 45%-75% B over 10 min) and dried by lyophilization. I-450 (19.9 mg, 16.33 μmol, 17.59% yield, 92.27% purity) as a white solid was obtained. LCMS (Method G): Rt=0.707, 0.733 min, [M+H]⁺=1124.8. SFC: Rt=2.575, 3.500 min. ¹H NMR (400 MHz, METHANOL-d4) δ=8.40-8.32 (m, 1H), 8.31-8.24 (m, 1H), 7.99-7.91 (m, 1H), 7.90-7.78 (m, 2H), 7.75-7.65 (m, 1H), 7.58-7.41 (m, 4H), 7.36 (br d, J=5.4 Hz, 1H), 7.20-7.11 (m, 1H), 7.08-6.88 (m, 3H), 5.89-5.77 (m, 1H), 4.69-4.60 (m, 1H), 4.41-4.34 (m, 2H), 4.12-3.88 (m, 3H), 3.88-3.62 (m, 8H), 3.60-3.41 (m, 7H), 3.19 (br d, J=8.4 Hz, 2H), 3.14 (br s, 1H), 2.80-2.64 (m, 3H), 2.34-2.15 (m, 3H), 2.11-2.02 (m, 1H), 1.98-1.63 (m, 12H), 1.60-1.46 (m, 3H), 1.38-1.18 (m, 5H), 1.16-0.92 (m, 3H). ¹⁹F NMR (377 MHz, METHANOL-d4) δ=−112.90-−113.52 (m, 1F), −116.22 (br s, 1F), −120.78 (br d, J=11.2 Hz, 1F).

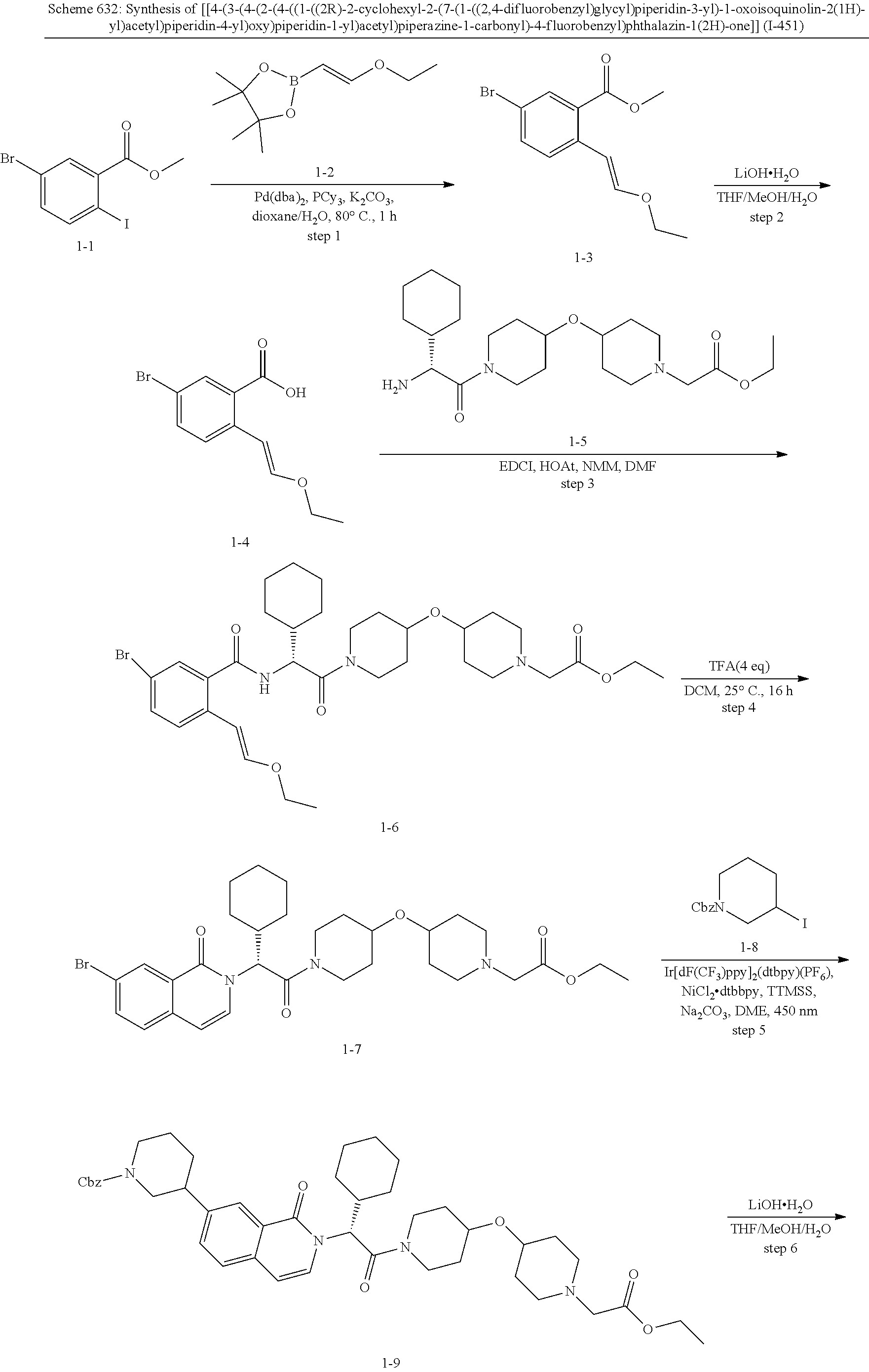

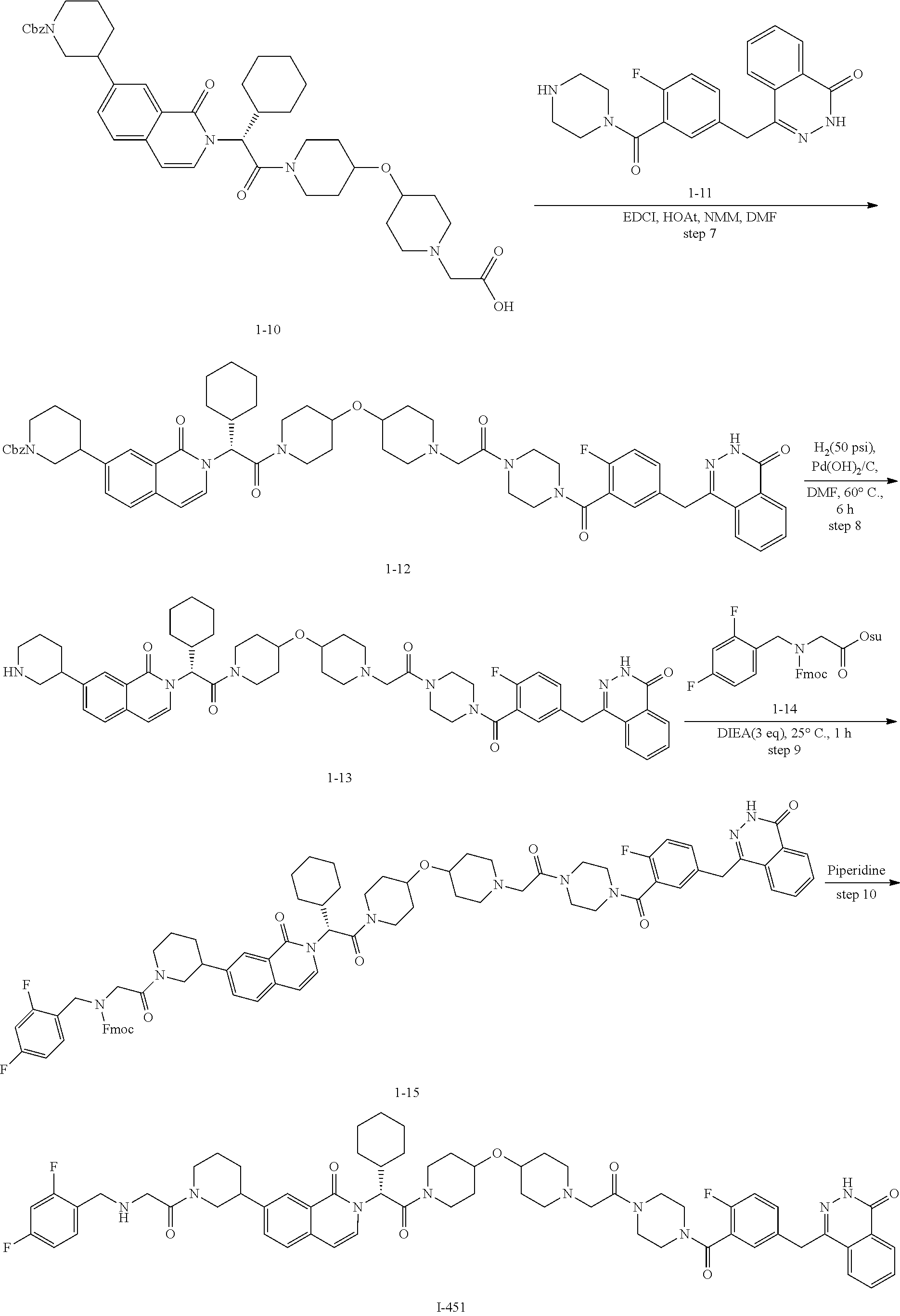

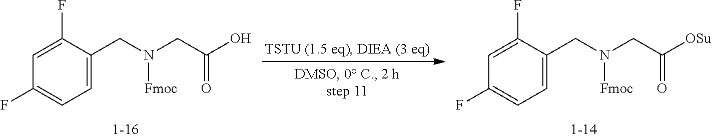

### Step 1: Synthesis of Intermediate 1-3

A mixture of Intermediate 1-1 (5 g, 14.67 mmol, 1 eq), Intermediate 1-2 (5.81 g, 29.33 mmol, 2 eq), (1E,4E)-1,5-diphenylpenta-1,4-dien-3-one;palladium (843.26 mg, 1.47 mmol, 0.1 eq), tricyclohexylphosphane (411.26 mg, 1.47 mmol, 475.44 μL, 0.1 eq) and dipotassium;carbonate (6.08 g, 44.00 mmol, 3 eq) in dioxane (50 mL) and H₂O (10 mL) was degassed and purged with N₂ 3 times, and then the mixture was stirred at 80° C. for 1 hr under N₂ atmosphere. The reaction mixture was diluted with H₂O (50 mL), and then extracted with EA (20 mL*3). The combined organic layers were washed with NaCl aq. (30 mL*2), dried over Na₂SO₄, filtered and concentrated under reduced pressure to give a residue. The residue was purified by flash silica gel chromatography (ISCO; 80 g SepaFlash Silica Flash Column, Eluent of 0˜10% Ethyl acetate/petroleum ether gradient @100 mL/min). The eluent was concentrated to give the product. Intermediate 1-3 (4.5 g, crude) was obtained as a yellow oil.

¹H NMR (400 MHz, CHLOROFORM-d) δ=8.03 (d, J=2.0 Hz, 1H), 7.80 (d, J=2.0 Hz, 1H), 7.56-7.60 (m, 1H), 7.50-7.54 (m, 1H), 7.36 (d, J=8.4 Hz, 1H), 7.08 (s, 1H), 7.05 (s, 1H), 6.97 (d, J=13.2 Hz, 1H), 6.70 (d, J=12.8 Hz, 1H), 4.46 (d, J=14.4 Hz, 2H), 4.40 (d, J=2.6 Hz, 1H), 4.30 (d, J=2.4 Hz, 1H), 4.01-3.94 (m, 3H), 3.91 (s, 3H), 1.40-1.34 (m, 4H).

### Step 2: Synthesis of Intermediate 1-4

To a solution of Intermediate 1-3 (4.5 g, 15.78 mmol, 1 eq) in THF (20 mL), MeOH (10 mL) and H₂O (20 mL) was added LiOH·H₂O (1.32 g, 31.56 mmol, 2 eq). The mixture was stirred at 25° C. for 3 h. The mixture was concentrated under vacuum to remove THF and MeOH. Then the pH was adjusted to 5 by citric acid and extracted with EA (10 ml*3). The combined organic layer was washed with brine (10 ml*3), dried over anhydrous Na₂SO₄, filtered and concentrated under vacuum to a crude product. The crude was used for the next step directly without purification. Intermediate 1-4 (4 g, crude) was obtained as a white solid.

### Step 3: Synthesis of Intermediate 1-6

To a solution of Intermediate 1-4 (4 g, 14.75 mmol, 1 eq) in DMF (40 mL) was added HOAt (2.01 g, 14.75 mmol, 2.06 mL, 1 eq), EDCI (5.66 g, 29.51 mmol, 2 eq), NMM (7.46 g, 73.77 mmol, 8.11 mL, 5 eq) and Intermediate 1-5 (3.29 g, 7.38 mmol, 0.5 eq, HCl salt). The mixture was stirred at 25° C. for 1 h. The reaction mixture was quenched by H₂O (100 mL) and extracted with EA (50 mL*3). The combined organic layers were washed with brine (50 mL*3), dried over anhydrous Na₂SO₄, filtered and concentrated under reduced pressure to give a residue. The crude product was purified by reversed-phase chromatography (0.1% NH₃·H₂O). Intermediate 1-6 (3.2 g, 4.83 mmol, 32.73% yield) was obtained as a yellow oil. LCMS (Method G): Rt=0.785 min, [M+H]⁺=662.3.

### Step 4: Synthesis of Intermediate 1-7

To a solution of Intermediate 1-6 (2.2 g, 3.32 mmol, 1 eq) in DCM (20 mL) was added TFA (1.51 g, 13.28 mmol, 986.46 μL, 4 eq) and the mixture was stirred at 25° C. for 16 hr. The mixture was adjusted to pH=7 with a Na₂CO₃ solution and extracted with DCM (20 mL*3). The organic layer was dried with anhydrous Na₂SO₄, filtered and concentrated under vacuum. The residue was purified by column chromatography (SiO₂, DCM/MeOH=1/0 to 10/1) and concentrated under vacuum. Intermediate 1-7 (1.4 g, 2.27 mmol, 68.39% yield) as a yellow oil was obtained. LCMS (Method G): Rt=0.780 min, [M+H]⁺=616.3.

### Step 5: Synthesis of Intermediate 1-9

A mixture of Intermediate 1-7 (1.6 g, 2.59 mmol, 1 eq), Intermediate 1-8 (1.17 g, 3.38 mmol, 1.30 eq), bis[3,5-difluoro-2-[5-(trifluoromethyl)-2-pyridyl]phenyl]iridium (1+); 4-tert-butyl-2-(4-tert-butyl-2-pyridyl) pyridine; hexafluorophosphate (2.91 g, 2.59 mmol, 1 eq), 4-tert-butyl-2-(4-tert-butyl-2-pyridyl) pyridine; dichloronickel (1.03 g, 2.59 mmol, 1 eq), bis(trimethylsilyl)silyl-trimethyl-silane (645.26 mg, 2.59 mmol, 800.57 μL, 1 eq) and disodium;carbonate (275.04 mg, 2.59 mmol, 1 eq) in DME (16 mL) was degassed and purged with N₂, and then the mixture was stirred at 25° C. for 16 hr and irradiated with a 455 nm blue LED. The mixture was diluted with water (10 mL) and extracted with EtOAc (20 mL*3). The organic layer was concentrated under vacuum. The residue was purified by prep-HPLC (column: Kromasil Eternity XT 250*80 mm*10 um; mobile phase: [water (NH₄HCO₃)-ACN]; gradient: 70%-100% B over 30 min) and dried by lyophilization. Intermediate 1-9 (600 mg, 794.75 μmol, 30.63% yield) as a yellow oil was obtained. LCMS (Method G): Rt=0.884 min, [M+H]⁺=755.5. SFC: Rt=1.641 min, 1.861 min. ¹H NMR (400 MHz, METHANOL-d₄) δ=8.16 (br d, J=12.3 Hz, 1H), 7.62-7.47 (m, 2H), 7.44 (br d, J=7.4 Hz, 1H), 7.40-7.31 (m, 5H), 6.75-6.67 (m, 1H), 5.83 (br t, J=9.6 Hz, 1H), 5.21-5.02 (m, 2H), 4.15 (qd, J=7.1, 9.5 Hz, 3H), 4.09-4.01 (m, 1H), 4.00-3.73 (m, 1H), 3.69-3.55 (m, 2H), 3.47 (td, J=3.8, 7.9 Hz, 1H), 3.45-3.38 (m, 2H), 3.20 (s, 1H), 3.15 (s, 1H), 2.85-2.67 (m, 3H), 2.41-2.19 (m, 3H), 1.88-1.76 (m, 7H), 1.71-1.58 (m, 4H), 1.50 (br d, J=3.8 Hz, 2H), 1.42-1.29 (m, 2H), 1.28-1.19 (m, 6H), 1.17-0.93 (m, 3H).

### Step 6: Synthesis of Intermediate 1-10

To a solution of Intermediate 1-9 (450 mg, 596.06 μmol, 1 eq) in THF (2 mL), MeOH (1 mL) and H₂O (1 mL) was added LiOH H₂O (75.03 mg, 1.79 mmol, 3 eq). The mixture was stirred at 25° C. for 1 hr. The mixture was concentrated under vacuum and the pH was adjusted to 6 with 1 M HCl solution and the mixture extracted with DCM (15 mL*3). The organic layer was dried with anhydrous Na₂SO₄, filtered and concentrated under vacuum. The residue was used in the next step without purification. Intermediate 1-10 (420 mg, 577.80 μmol, 96.94% yield) as a yellow solid was obtained. LCMS (Method G): Rt=0.595 min, [M+H]⁺=727.5. SFC: Rt=3.016 min, 3.509 min.

### Step 7: Synthesis of Intermediate 1-12

To a solution of Intermediate 1-10 (560 mg, 770.40 μmol, 1 eq) in DMF (6 mL) was added Intermediate 1-11 (282.26 mg, 770.40 μmol, 1 eq), EDCI (295.37 mg, 1.54 mmol, 2 eq), NMM (389.63 mg, 3.85 mmol, 423.51 μL, 5 eq) and HOAt (104.86 mg, 770.40 μmol, 107.77 μL, 1 eq). The mixture was stirred at 25° C. for 1 hr. The reaction mixture was diluted with water (20 ml), the precipitate was filtered and the filter cake was washed with water (5 mL*3). The filter cake was dried under vacuum to afford the product. Intermediate 1-12 (730 mg, 678.90 μmol, 88.12% yield) as a white solid was obtained. LCMS (Method G): Rt=0.811 min, [M+H]⁺=1076.7. SFC: Rt=0.976 min, 1.381 min.

### Step 8: Synthesis of Intermediate 1-13

To a solution of Pd(OH)₂/C (50.00 mg, 71.21 μmol, 20% purity, 7.66e−1 eq) in DMF (1 mL) was added a solution of Intermediate 1-12 (100 mg, 93.00 μmol, 1 eq) in DMF (1 mL). The suspension was degassed and purged with H₂ 3 times. The mixture was stirred under H₂ (50 psi) at 60° C. for 6 hr. The mixture was filtered through a pad of Celite and washed with DMF (1 mL*4). A solution of Intermediate 1-13 (85 mg, crude) in DMF (6 mL) as a brown liquid was obtained. LCMS (Method G): Rt=0.856 min, [M+H]⁺=941.5.

### Step 9: Synthesis of Intermediate 1-15

To a solution of Intermediate 1-13 (75 mg, 79.69 μmol, 1 eq) in DMF (6 mL) was added DIEA (30.90 mg, 239.07 μmol, 41.64 μL, 3 eq) and Intermediate 1-14 (62.22 mg, 119.54 μmol, 1.5 eq). The mixture was stirred at 25° C. for 1 hr. A solution of Intermediate 1-15 (100 mg, crude) in DMF (6 mL) as a brown liquid was obtained. LCMS (Method G): Rt=0.877 min, [M+H]⁺=1346.9.

### Step 10: Synthesis of I-451

To a solution of Intermediate 1-15 (100 mg, 74.26 μmol, 1 eq) in DMF (6 mL) was added piperidine (215.55 g, 2.53 μmol, 0.25 μL, 3.41e−2 eq). The mixture was stirred at 25° C. for 0.5 hr. The mixture was diluted with water (5 ml) and extracted with EtOAc (10 ml*3). The organic layer was concentrated under vacuum. The residue was purified by prep-HPLC (column: Waters Xbridge 150*25 mm*5 um; mobile phase: [water (NH₄HCO₃)-ACN]; gradient: 42%-72% B over 15 min) and dried by lyophilization. I-451 (35 mg, 31.13 μmol, 41.92% yield, 100% purity) was obtained as a white solid. LCMS (Method G): Rt=0.736 min, [M+H]⁺=1124.8. SFC: Rt=3.534 min, 3.971 min. ¹H NMR (400 MHz, DMSO-d₆) δ=12.62-12.56 (m, 1H), 8.28-8.22 (m, 1H), 8.15-8.09 (m, 1H), 7.99-7.92 (m, 1H), 7.91-7.78 (m, 2H), 7.70-7.60 (m, 2H), 7.51-7.45 (m, 1H), 7.44-7.34 (m, 3H), 7.26-7.20 (m, 1H), 7.20-7.11 (m, 1H), 7.09-6.98 (m, 1H), 6.72-6.66 (m, 1H), 5.76-5.65 (m, 1H), 4.48-4.40 (m, 1H), 4.32 (br d, J=4.4 Hz, 2H), 4.00-3.78 (m, 3H), 3.71 (br d, J=14.4 Hz, 2H), 3.59 (br d, J=1.6 Hz, 4H), 3.51-3.39 (m, 4H), 3.37-3.33 (m, 2H), 3.22-2.98 (m, 7H), 2.74-2.68 (m, 1H), 2.65-2.57 (m, 2H), 2.20-1.90 (m, 5H), 1.82-1.63 (m, 8H), 1.58 (br s, 3H), 1.44-1.20 (m, 5H), 1.18-1.01 (m, 5H), 0.90-0.84 (m, 1H). ¹⁹F NMR (377 MHz, DMSO-d₆) δ=−112.57-−112.73 (m, 1F), −114.93-−115.01 (m, 1F), −119.72-−119.88 (m, 1F).

### Step 11: Synthesis of Intermediate 1-14

To a solution of Intermediate 1-15 (200 mg, 472.36 μmol, 1 eq) in DMSO (2 mL) was added [dimethylamino-(2,5-dioxopyrrolidin-1-yl)oxy-methylene]-dimethyl-ammonium; tetra fluoroborate (213.30 mg, 708.54 μmol, 1.5 eq) and DIEA (183.15 mg, 1.42 mmol, 246.83 μL, 3 eq). The mixture was stirred at 0° C. for 2 hr. The mixture was diluted with water (2 mL), extracted with EtOAc (10 mL*3) and washed with brine (2 mL*3). The organic layer was dried with anhydrous Na₂SO₄, filtered and concentrated under vacuum. The residue was purified by column chromatography (SiO₂, Petroleum ether/Ethyl acetate=1/0 to 1/1) and concentrated under vacuum. Intermediate 1-14 (140 mg, crude) as a colorless oil was obtained. ¹H NMR (400 MHz, CHLOROFORM-d) δ=7.76 (d, J=7.5 Hz, 2H), 7.55 (br dd, J=7.5, 15.8 Hz, 2H), 7.40 (br t, J=7.3 Hz, 2H), 7.35-7.29 (m, 2H), 6.91-6.67 (m, 3H), 4.67-4.60 (m, 2H), 4.47 (d, J=7.0 Hz, 1H), 4.38-4.24 (m, 4H), 2.84 (br d, J=5.5 Hz, 4H). ¹⁹F NMR (377 MHz, CHLOROFORM-d) δ=−109.91 (br dd, J=6.7, 62.8 Hz, 1F), −112.37-−115.23 (m, 1F).

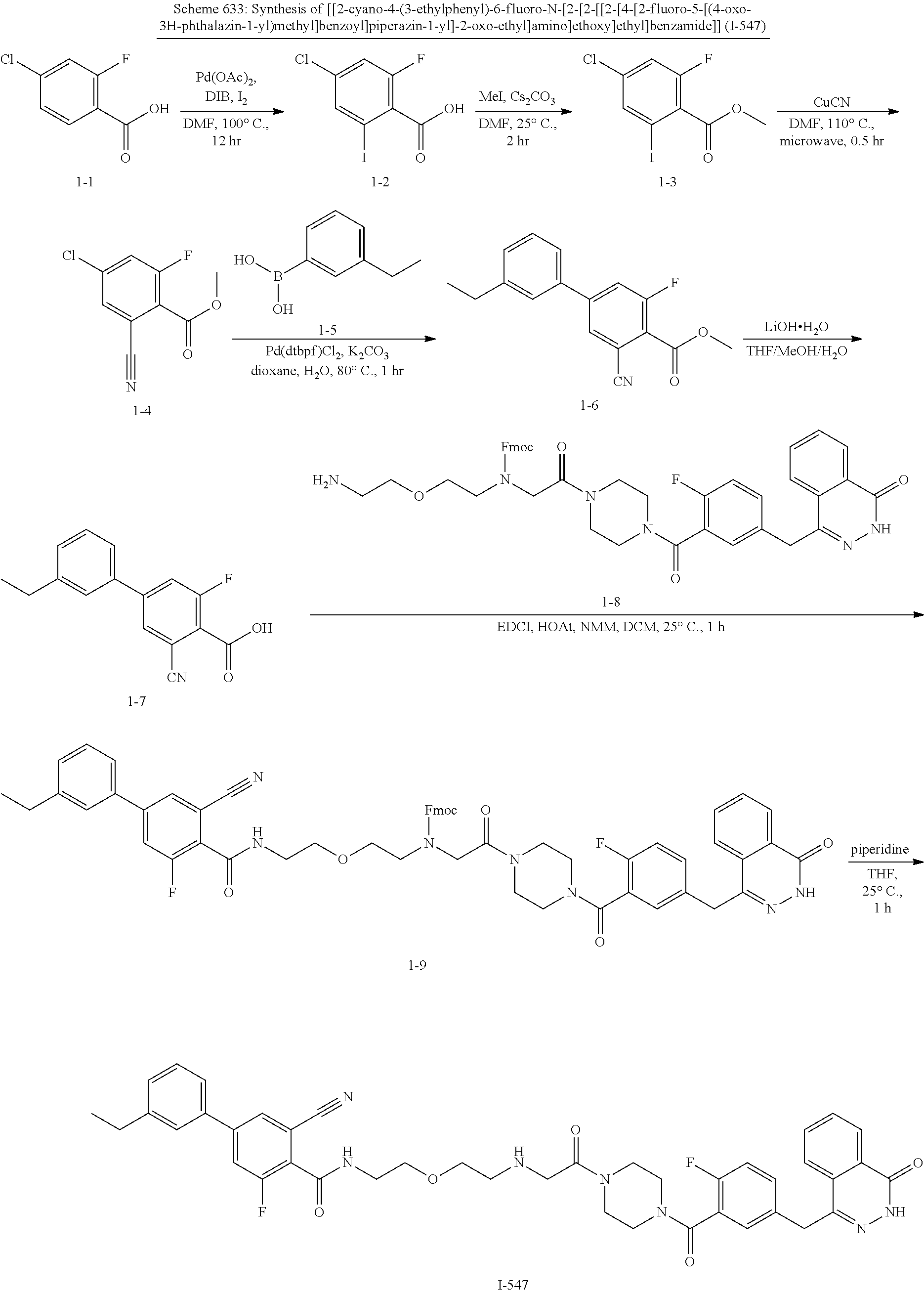

### Step 1: Synthesis of Intermediate 1-2

To a solution of Intermediate 1-1 (5 g, 28.64 mmol, 1 eq) in DMF (50 mL) was [acetoxy(phenyl)iodanyl]acetate (10.15 g, 31.51 mmol, 1.1 eq), I₂ (8.00 g, 31.51 mmol, 6.35 mL, 1.1 eq) and Pd (OAc)2 (321.54 mg, 1.43 mmol, 0.05 eq), then the mixture was stirred at 100° C. for 12 hours under N₂. The mixture was diluted with 1N HCl (30 mL) and extracted with EA (30 mL*3). The combined organic layer was dried over Na₂SO₄, filtered and concentrated in vacuo to give Intermediate 1-2 (10 g, crude) as a brown oil.

### Step 2: Synthesis of Intermediate 1-3

To a solution of Intermediate 1-2 (10 g, 16.64 mmol, 1 eq) in DMF (120 mL) was added iodomethane (2.36 g, 16.64 mmol, 1.04 mL, 1 eq) and Cs₂CO₃ (6.51 g, 19.97 mmol, 1.2 eq). The mixture was stirred at 25° C. for 2 hr. The mixture was diluted with water (240 mL) and extracted with EA (80 mL*3). The combined organic layer was dried over Na₂SO₄, filtered and concentrated in vacuo to give a residue. The residue was purified by flash silica gel chromatography (ISCO; 220 g SepaFlash Silica Flash Column, Eluent of 0˜10% EA: PEgradient @150 mL/min). The eluent was concentrated under reduced pressure to give Intermediate 1-3 (4.5 g, 14.31 mmol, 85.99% yield) as a yellow oil. ¹H NMR (400 MHz, CHLOROFORM-d) δ=7.90 (s, 1H), 7.15 (s, 1H), 3.94 (s, 3H). ¹⁹F NMR (376 MHz, METHANOL-d4) δ=−108.643.

### Step 3: Synthesis of Intermediate 1-4

To a solution of Intermediate 1-3 (3.5 g, 11.13 mmol, 1 eq) in DMF (35 mL) was added CuCN (996.80 mg, 11.13 mmol, 2.43 mL, 1 eq). The mixture was stirred at 110° C. for 2 hr. The reaction mixture was diluted with water (120 mL) and extracted with ethyl acetate (40 mL×3). The combined organic layers were washed with brine (40 mL×3) and dried over sodium sulfate, filtered and concentrated under reduced pressure to give a residue. The residue was purified by flash silica gel chromatography (ISCO; 80 g SepaFlash Silica Flash Column, Eluent of 0˜10% Methanol: Ethyl acetate gradient @80 mL/min). The eluent was concentrated under reduced pressure to give Intermediate 1-4 (1.7 g, 6.96 mmol, 62.49% yield, 87.386% purity) as a white solid. LCMS (Method D): Rt: 0.350 min, [M+H]⁺=214.0. ¹H NMR (400 MHz, CHLOROFORM-d) δ=7.60-7.58 (m, 1H), 7.46-7.42 (m, 1H), 4.03 (s, 3H). ¹⁹F NMR (376 MHz, METHANOL-d4) δ=−104.507.

### Step 4: Synthesis of Intermediate 1-6

To a solution of Intermediate 1-5 (280.88 mg, 1.87 mmol, 1 eq) and Intermediate 1-4 (400 mg, 1.87 mmol, 1 eq) in H₂O (1.5 mL) and dioxane (6 mL) was added Pd(dtbpf)Cl₂ (137.03 mg, 187.27 μmol, 0.1 eq) and K₂CO₃ (776.46 mg, 5.62 mmol, 3 eq). The suspension was degassed and purged with N₂ 3 times. The mixture was stirred at 90° C. for 1 hr under N₂. The reaction mixture was diluted with water (60 mL) and extracted with ethyl acetate (20 mL×3). The combined organic layers were washed with brine (20 mL×3) and dried over sodium sulfate, filtered and concentrated under reduced pressure to give a residue. The residue was purified by flash silica gel chromatography (ISCO; 40 g SepaFlash Silica Flash Column, Eluent of 0˜20% Methanol: Ethyl acetate gradient @60 mL/min). The eluent was concentrated under reduced pressure to give Intermediate 1-6 (420 mg, 1.37 mmol, 73.31% yield, 92.6% purity) as a yellow oil. LCMS (Method D): Rt: 0.492 min, [M+H]⁺=284.1.

### Step 5: Synthesis of Intermediate 1-7

To a solution of Intermediate 1-6 (400 mg, 1.41 mmol, 1 eq) in H₂O (0.4 mL), THF (1 mL) and MeOH (1 mL) was added LiOH·H₂O (177.75 mg, 4.24 mmol, 3 eq). The mixture was stirred at 40° C. for 0.5 hr. The mixture was concentrated to give a residue. The reaction mixture was diluted with water (60 mL). Then the mixture was adjusted to pH=6-7 with a saturated citric acid aqueous solution. Then the reaction mixture was extracted with DCM (20 mL×3). The combined organic layers were washed with brine (20 mL×3) and dried over sodium sulfate, filtered and concentrated under reduced pressure to give Intermediate 1-7 (350 mg, 1.30 mmol, 92.06% yield) as a white solid. LCMS (Method D): Rt: 0.378 min, [M+H]⁺=270.9.

### Step 6: Synthesis of Intermediate 1-9

To a solution of Intermediate 1-7 (50 mg, 185.69 μmol, 1 eq) in DCM (2 mL) was added EDCI (106.79 mg, 557.06 μmol, 3 eq), NMM (93.91 mg, 928.44 μmol, 102.07 μL, 5 eq) and HOAt (25.27 mg, 185.69 μmol, 25.98 μL, 1 eq) at 25° C. for 0.5 hr. Then Intermediate 1-8 (136.07 mg, 185.69 μmol, 1 eq) was added into the mixture and it was stirred at 25° C. for 0.5 hr. The reaction mixture was diluted with water (30 mL) and extracted with ethyl acetate (10 mL×3). The combined organic layers were washed with brine (10 mL×3) and dried over sodium sulfate, filtered and concentrated under reduced pressure to give a residue. The crude product was purified by prep-HPLC (column: CD20-Waters Xbidge BEH C18 250*25*10 um; mobile phase: [water (NH₄HCO₃)-ACN]; gradient: 54%-84% B over 15 min) and the eluent was concentrated under reduced pressure to remove ACN and then lyophilized to give Intermediate 1-9 (20 mg, 19.98 μmol, 10.76% yield, 98.285% purity) as a white solid. LCMS (Method H): Rt: 0.779 min, [M+H]⁺=984.4.

### Step 7: Synthesis of I-547

To a solution of Intermediate 1-9 (20 mg, 20.32 μmol, 1 eq) in THF (0.5 mL) was added piperidine (0.4 mL). The mixture was stirred at 25° C. for 0.5 hr. The reaction mixture was diluted with water (30 mL) and extracted with ethyl acetate (10 mL×3). The combined organic layers were washed with brine (10 mL×3) and dried over sodium sulfate, filtered and concentrated under reduced pressure to give a residue. The crude product was purified by prep-HPLC (column: CD20-Waters Xbidge BEH C18 250*25*10 um; mobile phase: [water (NH₄HCO₃)-ACN]; gradient: 36%-56% B over 15 min) and the eluent was concentrated under reduced pressure to remove ACN and then lyophilized to give I-547 (1.07 mg, 1.35 μmol, 6.65% yield, 96.286% purity) as a white solid. LCMS (Method H): Rt: 0.628 min, [M+H]⁺=762.5. ¹H NMR (400 MHz, METHANOL-d4) δ=8.37 (d, J=7.6 Hz, 1H), 8.08 (s, 1H), 7.99-7.92 (m, 1H), 7.90-7.79 (m, 2H), 7.64-7.58 (m, 1H), 7.58-7.54 (m, 1H), 7.53-7.46 (m, 2H), 7.46-7.41 (m, 1H), 7.40-7.30 (m, 3H), 7.30-7.22 (m, 1H), 7.20-7.11 (m, 1H), 4.39 (d, J=4.0 Hz, 2H), 3.80 (s, 4H), 3.71 (d, J=4.8 Hz, 1H), 3.65 (s, 3H), 3.58-3.51 (m, 2H), 3.50-3.44 (m, 2H), 3.41-3.35 (m, 2H), 3.24-3.16 (m, 1H), 2.85-2.77 (m, 2H), 2.77-2.67 (m, 2H), 1.31-1.26 (m, 3H). ¹⁹F NMR (376 MHz, METHANOL-d4) δ=−117.754, 120.704.

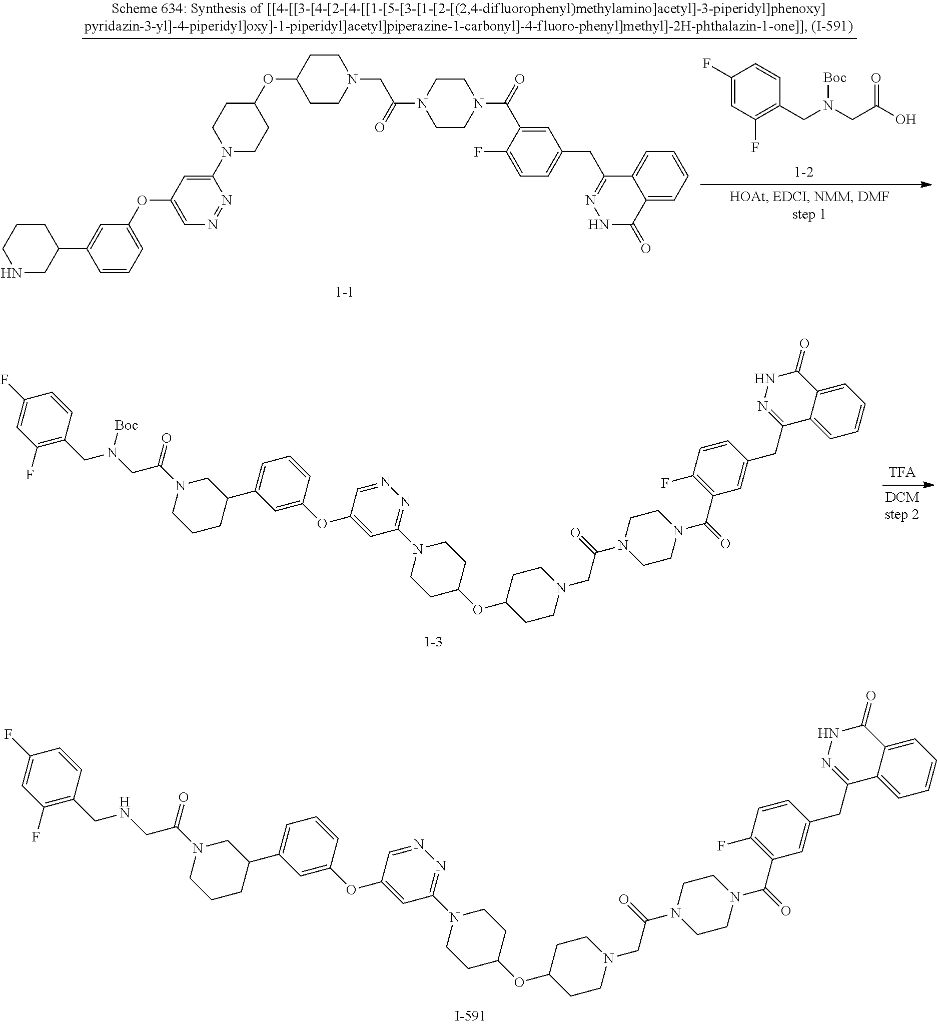 [SMILES: O=C(CN1CCC(OC2CCN(c3cc(Oc4cccc(C5CCCNC5)c4)cnn3)CC2)CC1)N1CCN(C(=O)c2cc(Cc3n[nH]c(=O)c4ccccc34)ccc2F)CC1 ; CC(C)(C)OC(=O)N(CC(=O)O)Cc1ccc(F)cc1F ; CC(C)(C)OC(=O)N(CC(=O)N1CCCC(c2cccc(Oc3cnnc(N4CCC(OC5CCN(CC(=O)N6CCN(C(=O)c7cc(Cc8n[nH]c(=O)c9ccccc89)ccc7F)CC6)CC5)CC4)c3)c2)C1)Cc1ccc(F)cc1F ; O=C(CN1CCC(OC2CCN(c3cc(Oc4cccc(C5CCCN(C(=O)CNCc6ccc(F)cc6F)C5)c4)cnn3)CC2)CC1)N1CCN(C(=O)c2cc(Cc3n[nH]c(=O)c4ccccc34)ccc2F)CC1]

### Step 1: Synthesis of Intermediate 1-3

To a solution of intermediate 1-1 (100 mg, 113.58 μmol, 1 eq, HCl salt) in DMF (1 mL) was added NMM (57.44 mg, 567.89 μmol, 62.44 μL, 5 eq) at 25° C., and the mixture was stirred at 25° C. for 10 min. Then intermediate 1-2 (34.22 mg, 113.58 μmol, 1 eq), EDCI (108.87 mg, 567.89 μmol, 5 eq) and HOAt (30.92 mg, 227.16 μmol, 31.78 μL, 2 eq) were added at 25° C. The resulting mixture was stirred at 25° C. for 1 hr. The reaction mixture was diluted with water (1 mL) and extracted with DCM (1 mL*3), the combined organic phase was dried by Na₂SO₄, and concentrated under reduced pressure to give a residue. The residue was purified by reversed-phase chromatography (0.1% of TFA) and the eluent was lyophilized to give product. Intermediate 1-3 (60 mg, 48.00 μmol, 42.26% yield, 90.184% purity) was obtained as a white solid. LCMS (Method D): Retention time: 0.430 min, [M+H]+=1127.8.

### Step 2: Synthesis of I-591

To a solution of intermediate 1-3 (60 mg, 53.23 μmol, 1 eq) in DCM (0.3 mL) was added TFA (460.50 mg, 4.04 mmol, 0.3 mL, 75.88 eq) and the mixture was stirred at 25° C. for 30 min. The mixture was concentrated under reduced pressure to give a residue. The residue was purified by reversed-phase chromatography (0.1% of TFA) and the eluent was lyophilized to give product. I-591 (15 mg, 12.85 mol, 24.15% yield, 97.776% purity, TFA salt) was obtained as a white solid. LCMS (Method D): Retention time: 0.295 min, [M+H]+=1027.3. ¹H NMR (400 MHz, METHANOL-d₄) δ=8.40-8.34 (m, 1H), 8.29 (d, J=4.8 Hz, 1H), 7.98-7.92 (m, 1H), 7.91-7.80 (m, 2H), 7.65-7.57 (m, 1H), 7.55-7.48 (m, 2H), 7.40-7.31 (m, 2H), 7.25-7.19 (m, 1H), 7.19-7.04 (m, 4H), 6.83-6.78 (m, 1H), 4.63-4.51 (m, 1H), 4.39 (s, 2H), 4.34-4.29 (m, 3H), 4.27-4.07 (m, 3H), 3.95-3.70 (m, 8H), 3.59-3.41 (m, 6H), 3.36 (d, J=5.2 Hz, 2H), 3.28-3.12 (m, 2H), 2.92-2.71 (m, 2H), 2.35-2.14 (m, 1H), 2.14-1.93 (m, 6H), 1.93-1.57 (m, 6H), 1.57-1.20 (m, 1H). ¹⁹F NMR (377 MHz, METHANOL-d₄), δ=−76.91 (s, 11F), −108.55-−108.30 (m, 1F), −112.78-−112.98 (m, 1F), −120.61-−120.83 (m, 2F).

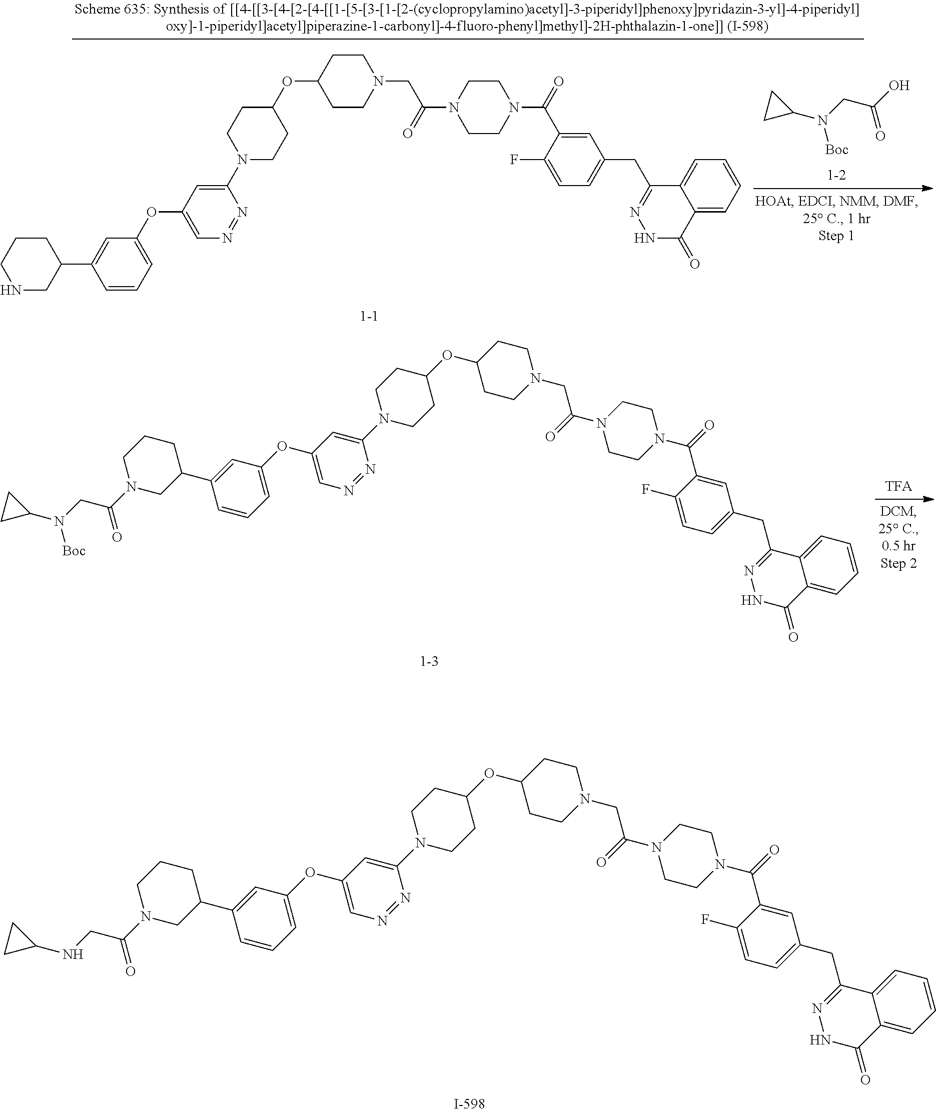 [SMILES: O=C(CN1CCC(OC2CCN(c3cc(Oc4cccc(C5CCCNC5)c4)cnn3)CC2)CC1)N1CCN(C(=O)c2cc(Cc3n[nH]c(=O)c4ccccc34)ccc2F)CC1 ; CC(C)(C)OC(=O)N(CC(=O)O)C1CC1 ; CC(C)(C)OC(=O)N(CC(=O)N1CCCC(c2cccc(Oc3cnnc(N4CCC(OC5CCN(CC(=O)N6CCN(C(=O)c7cc(Cc8n[nH]c(=O)c9ccccc89)ccc7F)CC6)CC5)CC4)c3)c2)C1)C1CC1 ; O=C(CN1CCC(OC2CCN(c3cc(Oc4cccc(C5CCCN(C(=O)CNC6CC6)C5)c4)cnn3)CC2)CC1)N1CCN(C(=O)c2cc(Cc3n[nH]c(=O)c4ccccc34)ccc2F)CC1]

### Step 1: Synthesis of Intermediate 1-3.

To a solution of Intermediate 1-1 (100 mg, 113.58 μmol, 1 eq, HCl salt) and Intermediate 1-2 (24.45 mg, 113.58 μmol, 1 eq) in DMF (1 mL) was added EDCI (65.32 mg, 340.74 μmol, 3 eq), HOAt (15.46 mg, 113.58 μmol, 15.89 μL, 1 eq) and NMM (57.44 mg, 567.89 μmol, 5 eq). The mixture was stirred at 25° C. for 1 hr. The mixture was quenched by addition of H₂O (1 mL) at 25° C., and extracted with DCM (1 mL*3), the combined organic phase was dried over Na₂SO₄, filtered and concentrated under reduced pressure to give a crude product. The crude product was purified by reversed-phase chromatography (0.1% FA condition), the eluent was concentrated to remove ACN and lyophilized to give the product. Intermediate 1-3 (50 mg, 44.31 μmol, 39.01% yield, 92.275% purity) was obtained as a red solid. LCMS (Method D): Rt=0.357 min, [M+H]⁺=1041.6.

### Step 2: Synthesis of I-598.

To a solution of Intermediate 1-3 (20 mg, 19.21 μmol, 1 eq) in DCM (0.5 mL) was added TFA (153.50 mg, 1.35 mmol, 0.1 mL). The mixture was stirred at 25° C. for 0.5 hr. The mixture was dried with a stream of N₂, 10 mL of H₂O was added and the mixture was lyophilized to give the product. I-598 (12.22 mg, 10.78 μmol, 56.10% yield, 93.039% purity, TFA salt) was obtained as a red solid. LCMS (Method D): Rt=0.283 min, [M+H]⁺=941.3. SFC: Retention time: 8.453 min, 9.906 min. ¹H NMR (400 MHz, METHANOL-d₄) δ=8.41-8.34 (m, 1H), 8.31 (s, 1H), 7.99-7.91 (m, 1H), 7.91-7.81 (m, 2H), 7.58-7.46 (m, 2H), 7.42-7.31 (m, 2H), 7.31-7.12 (m, 3H), 6.92-6.80 (m, 1H), 4.62-4.53 (m, 1H), 4.39 (s, 2H), 4.33-4.17 (m, 4H), 4.02 (s, 2H), 3.88-3.75 (m, 6H), 3.74-3.65 (m, 2H), 3.60-3.49 (m, 4H), 3.48-3.40 (m, 2H), 3.25-3.11 (m, 2H), 2.95-2.73 (m, 4H), 2.25-2.15 (m, 1H), 2.12-1.93 (m, 6H), 1.92-1.69 (m, 5H), 1.66-1.54 (m, 1H), 0.92-0.90 (m, 4H). ¹⁹F NMR (376 MHz, METHANOL-d₄) δ=−77.083, −120.763.

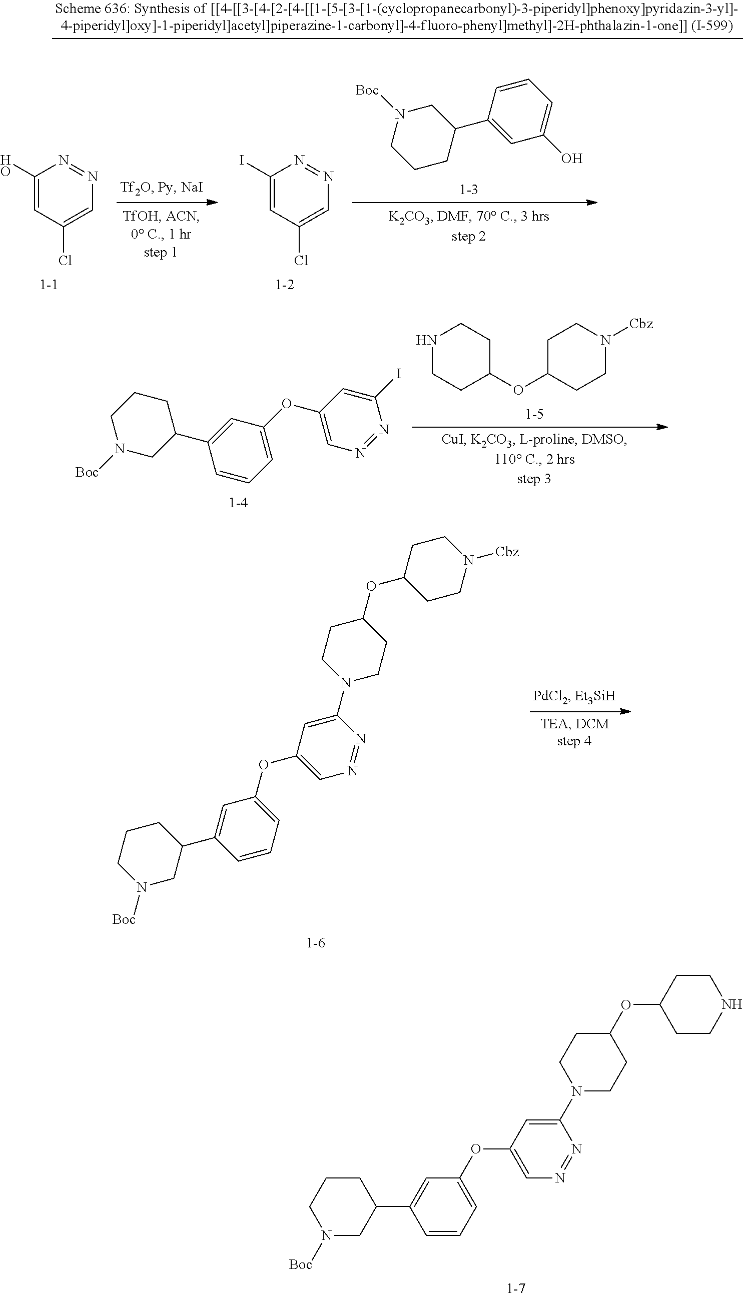

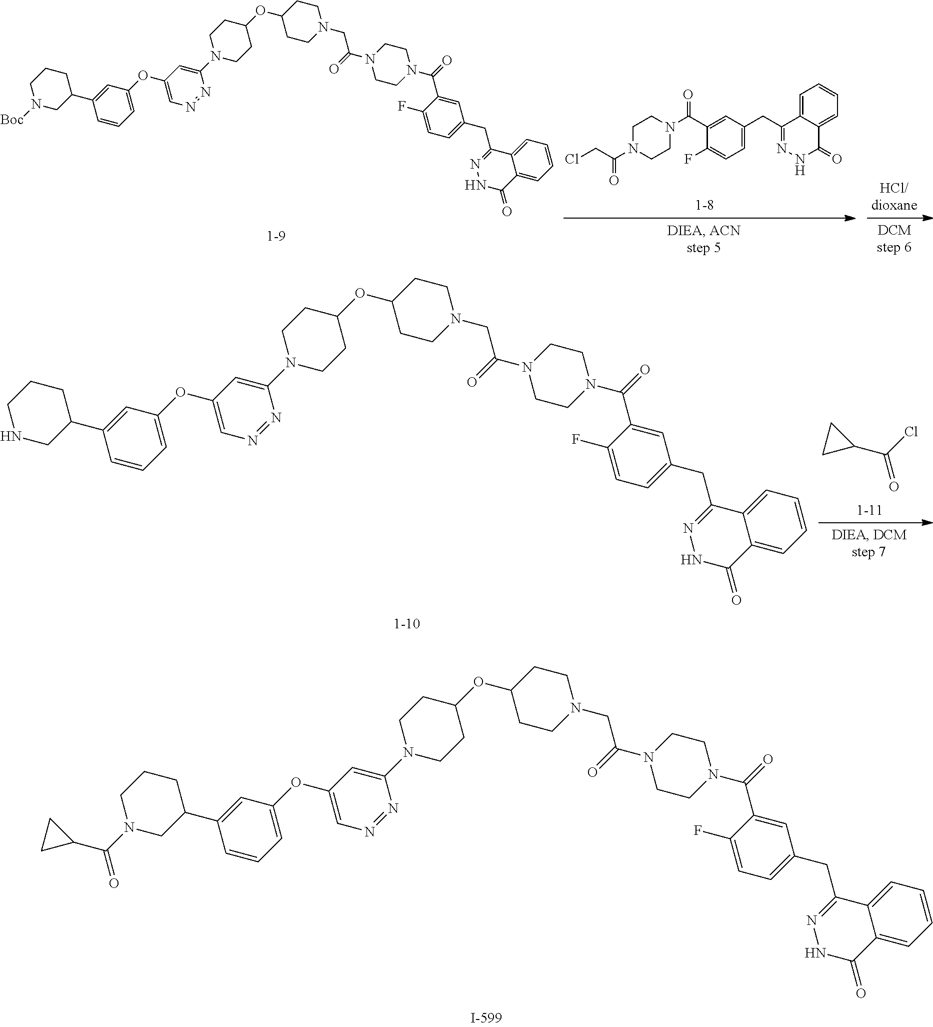

### Step 1: Synthesis of Intermediate 1-3

To a solution of pyridine (1.82 g, 22.98 mmol, 1.86 mL, 1.2 eq) and intermediate 1-1 (2.5 g, 19.15 mmol, 1 eq) in ACN (5 mL) was added Tf₂O (4.59 g, 16.28 mmol, 2.69 mL, 0.85 eq) dropwise at 0° C. The mixture was stirred for 30 min, then charged with NaI (2.87 g, 19.15 mmol, 1 eq) in one portion. TfOH (3.16 g, 21.07 mmol, 1.86 mL, 1.1 eq) was added dropwise and the mixture was stirred for 1 h at 0° C. The pH of the reaction mixture was neutralized to 7-8 with aq. NaHCO₃ and the mixture was extracted with EA (25 mL*3). The combined organic layer was dried over Na₂SO₄, filtered and concentrated in vacuo to give a residue. The residue was purified by flash silica gel chromatography (ISCO; 80 g SepaFlash Silica Flash Column, Eluent of 0˜50% Ethyl acetate/Petroleum ether @60 mL/min) and the eluent was concentrated to give product. Intermediate 1-2 (2.358 g, 8.72 mmol, 45.51% yield, 88.876% purity) was obtained as a white solid. LCMS (Method D): Retention time: 0.253 min, [M+H]⁺=240.7.

### Step 2: Synthesis of Intermediate 1-4

To a solution of intermediate 1-2 (2.358 g, 9.81 mmol, 1 eq) and intermediate 1-3 (2.72 g, 9.81 mmol, 1 eq) in DMF (23 mL) was added K₂CO₃ (2.71 g, 19.61 mmol, 2 eq) at 25° C. The mixture was stirred at 60° C. for 1 hr. The reaction mixture was diluted with water (20 mL) and extracted with DCM (20 mL*3). The combined organic phase was dried by Na₂SO₄, and concentrated under reduced pressure to give a residue. The residue was purified by flash silica gel chromatography (ISCO; 80 g SepaFlash Silica Flash Column, Eluent of 0˜50% Ethyl acetate/Petroleum ether @70 mL/min) and the eluent was concentrated to give product. Intermediate 1-4 (4.21 g, 8.34 mmol, 85.02% yield, 95.330% purity) was obtained as a white solid. LCMS (Method D): Retention time: 0.484 min, [M+H]⁺=482.0.

### Step 3: Synthesis of Intermediate 1-6

To a solution of intermediate 1-4 (1 g, 2.08 mmol, 1.1 eq) and intermediate 1-5 (601.39 mg, 1.89 mmol, 1 eq) in DMSO (10 mL) was added CuI (35.97 mg, 188.87 μmol, 0.1 eq), K₂CO₃ (783.10 mg, 5.67 mmol, 3 eq) and (2S)-pyrrolidine-2-carboxylic acid (43.49 mg, 377.74 μmol, 0.2 eq). The mixture was stirred at 100° C. for 3 hr. The reaction mixture was filtered to give the filtrate. The filtrate was concentrated, and the residue was purified by reversed-phase chromatography (0.1% of FA) and the eluent was lyophilized to give product. Intermediate 1-6 (717 mg, 1.04 mmol, 55.18% yield, 97.651% purity) was obtained as a white solid. LCMS (Method D): Retention time: 0.456 min, [M+H]⁺=672.3.

### Step 4: Synthesis of Intermediate 1-7

To a solution of intermediate 1-6 (530 mg, 788.90 μmol, 1 eq) in DCM (5.3 mL) was added PdCl₂ (69.95 mg, 394.45 μmol, 0.5 eq) and TEA (239.48 mg, 2.37 mmol, 329.41 μL, 3 eq). The reaction mixture was degassed and backfilled with N₂ three times. Then Et₃SiH (1.10 g, 9.47 mmol, 1.51 mL, 12 eq) was added. The reaction mixture was stirred at 25° C. for 1 hr. The reaction mixture was diluted with water (5 mL) and extracted with DCM (5 mL*3). The combined organic phase was dried by Na₂SO₄, and concentrated under reduced pressure to give a residue. The residue was purified by flash silica gel chromatography (ISCO; 20 g Sepa Flash SilicaFlash Column, Eluent of 0-50% Methanol/Dichloromethane @50 mL/min) and the eluent was concentrated to give product. Intermediate 1-7 (430 mg, 578.32 mol, 73.31% yield, 72.316% purity) was obtained as a yellow solid. LCMS (Method D): Retention time: 0.322 min, [M+H]⁺=538.3.

### Step 5: Synthesis of Intermediate 1-9

To a solution of intermediate 1-7 (410 mg, 762.52 μmol, 1 eq) and intermediate 1-8 (337.70 mg, 762.52 μmol, 1 eq) in ACN (4.1 mL) was added DIEA (197.10 mg, 1.53 mmol, 265.63 μL, 2 eq). The mixture was stirred at 60° C. for 1 hr. The reaction mixture was diluted with water (4 mL) and extracted with DCM (4 mL*3). The combined organic phase was dried by Na₂SO₄, and concentrated under reduced pressure to give a residue. The residue was purified by flash silica gel chromatography (ISCO; 20 g SepaFlash SilicaFlash Column, Eluent of 0˜50% Ethyl acetate/Petroleum ether @40 mL/min) and the eluent was concentrated to give product. Intermediate 1-9 (405 mg, 326.62 μmol, 42.83% yield, 76.138% purity) was obtained as a yellow solid. LCMS (Method D): Retention time: 0.371 min, [M+H]⁺=944.3.

### Step 6: Synthesis of Intermediate 1-10

A mixture of intermediate 1-9 (400 mg, 423.68 μmol, 1 eq) and HCl/dioxane (2 M, 4.00 mL) was stirred at 25° C. for 1 hr. The mixture concentrated under reduced pressure to give a residue. The crude product was used in the next step without further purification. Intermediate 1-10 (400 mg, crude, HCl salt) was obtained as a yellow solid. LCMS (Method D): Retention time: 0.264 min, [M+H]⁺=844.5.

### Step 7: Synthesis of I-599

To a mixture of intermediate 1-10 (70 mg, 79.51 μmol, 1 eq, HCl salt) and intermediate 1-11 (9.97 mg, 95.41 μmol, 8.66 μL, 1.2 eq) in DCM (0.7 mL) was added DIEA (20.55 mg, 159.01 μmol, 27.70 L, 2 eq) at 25° C. The mixture was stirred at 25° C. for 1 hr. The reaction mixture was washed with water (1 mL) and extracted with DCM (1 mL*3). The combined organic phase was dried by Na₂SO₄, and concentrated under reduced pressure to give a residue. The crude was purified by prep-HPLC (column: CD01-Phenomenex luna C18 150*25*10 um; mobile phase: [water(FA)-ACN]; gradient: 12%-42% B over 10 min). The eluent was concentrated to remove ACN and lyophilized to give product. I-599 (13 mg, 13.54 μmol, 17.03% yield, 99.773% purity, FA salt) was obtained as a white solid. LCMS (Method D): Retention time: 0.335 min, [M+H]⁺=912.3. ¹H NMR (400 MHz, METHANOL-d₄) δ=8.47 (s, 1H), 8.40-8.34 (m, 1H), 8.27 (d, J=2.0 Hz, 1H), 7.99-7.92 (m, 1H), 7.90-7.81 (m, 2H), 7.53-7.47 (m, 1H), 7.47-7.42 (m, 1H), 7.37 (t, J=6.0 Hz, 1H), 7.32-7.23 (m, 1H), 7.20-7.10 (m, 2H), 7.09-7.01 (m, 1H), 6.56-6.47 (m, 1H), 4.60-4.46 (m, 1H), 4.39 (s, 3H), 3.97-3.87 (m, 2H), 3.82-3.72 (m, 3H), 3.67 (d, J=6.0 Hz, 4H), 3.55-3.33 (m, 5H), 3.29-3.19 (m, 2H), 3.06-2.90 (m, 2H), 2.87-2.52 (m, 4H), 2.12-1.76 (m, 9H), 1.75-1.48 (m, 5H), 0.90-0.75 (m, 4H). ¹⁹F NMR (377 MHz, METHANOL-d₄), δ=120.779 (s, 1F).

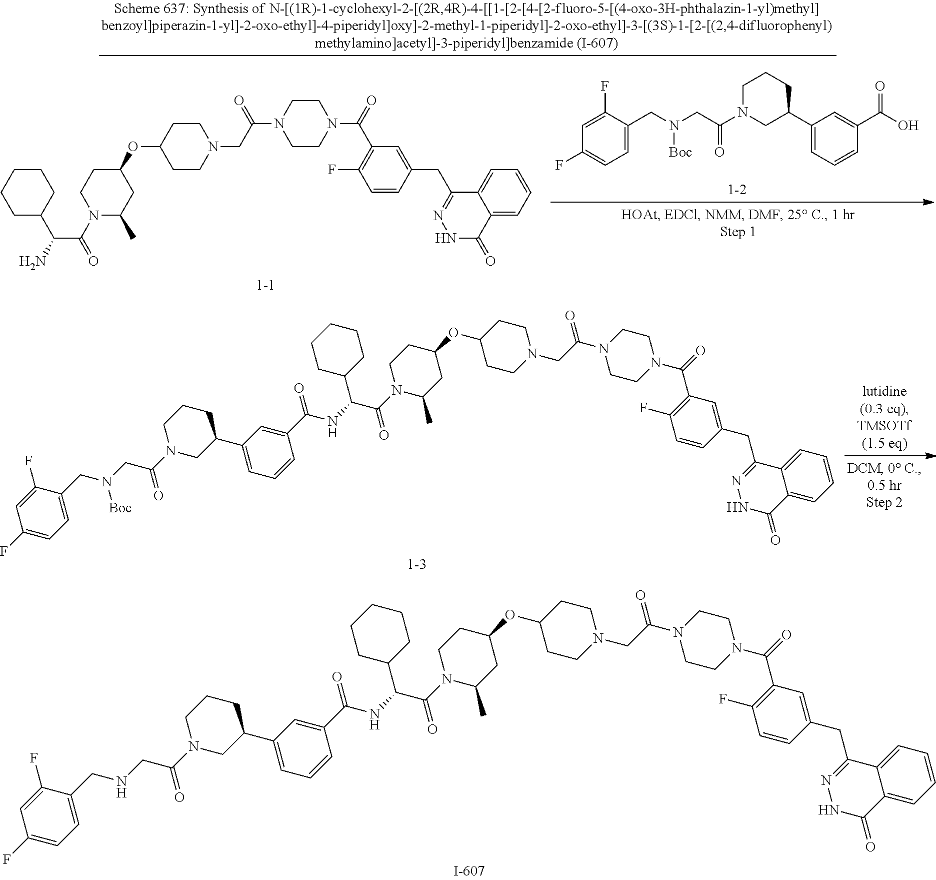

### Step 1: Synthesis of Intermediate 1-3

To a solution of Intermediate 1-1 (76.14 mg, 97.57 μmol, 9.53e−1 eq, HCl salt) in DMF (1 mL) was added Intermediate 1-2 (50 mg, 102.35 μmol, 1 eq), EDCI (58.86 mg, 307.05 μmol, 3 eq), HOAt (13.93 mg, 102.35 μmol, 14.32 μL, 1 eq) and NMM (51.76 mg, 511.75 μmol, 56.26 μL, 5 eq). The mixture was stirred at 25° C. for 1 hr. The reaction mixture was diluted with H₂O (1 mL) and extracted with EA (2 mL*2). The combined organic layers were washed with brine (2 mL*1), dried over Na₂SO₄, filtered and concentrated under reduced pressure to give a residue. The crude product was purified by reversed-phase chromatography (0.1% FA condition). The eluent was concentrated under reduced pressure to remove ACN and then lyophilized to give Intermediate 1-3 (50 mg, 39.59 μmol, 38.68% yield, 96.157% purity) as a white solid. LCMS (Method D): Rt=0.478 min, [M+H]⁺=1214.5. SFC: Rt=0.2030 min.

### Step 2: Synthesis of I-607

To a solution of Intermediate 1-3 (45 mg, 37.05 μmol, 1 eq) in DCM (0.5 mL) was added 2,6-dimethylpyridine (1.19 mg, 11.12 μmol, 1.29 μL, 0.3 eq) and TMSOTf (12.35 mg, 55.58 μmol, 10.04 μL, 1.5 eq). The mixture was stirred at 0° C. for 10 min. The pH was basified to 7-8 with NH₃·H₂O and the mixture was concentrated in vacuo. The crude product was purified by reversed-phase chromatography (0.1% NH₃·H₂O condition). The eluent was concentrated under reduced pressure to remove ACN and then lyophilized to give I-607 (10.76 mg, 9.66 μmol, 26.06% yield, 100% purity) as a white solid. LCMS (Method D): Rt=0.371 min, [M+H]⁺=1114.4. SFC: Rt=4.379 min. ¹H NMR (400 MHz, METHANOL-d₄) δ=8.37 (d, J=7.2 Hz, 1H), 7.99-7.92 (m, 1H), 7.91-7.81 (m, 2H), 7.78-7.74 (m, 1H), 7.73-7.68 (m, 1H), 7.51-7.36 (m, 5H), 7.20-7.13 (m, 1H), 7.00-6.88 (m, 2H), 4.72-4.49 (m, 3H), 4.39 (s, 2H), 3.92-3.86 (m, 1H), 3.85-3.78 (m, 4H), 3.76-3.70 (m, 2H), 3.67-3.62 (m, 1H), 3.59-3.41 (m, 6H), 3.28-3.25 (m, 2H), 3.21-3.10 (m, 3H), 2.83-2.63 (m, 4H), 2.40-2.25 (m, 2H), 2.07-2.01 (m, 1H), 1.91-1.76 (m, 10H), 1.73-1.50 (m, 8H), 1.35-1.18 (m, 6H), 1.15-1.02 (m, 2H). ¹⁹F NMR (377 MHz, METHANOL-d₄) δ=−113.409, −116.231, −120.794.

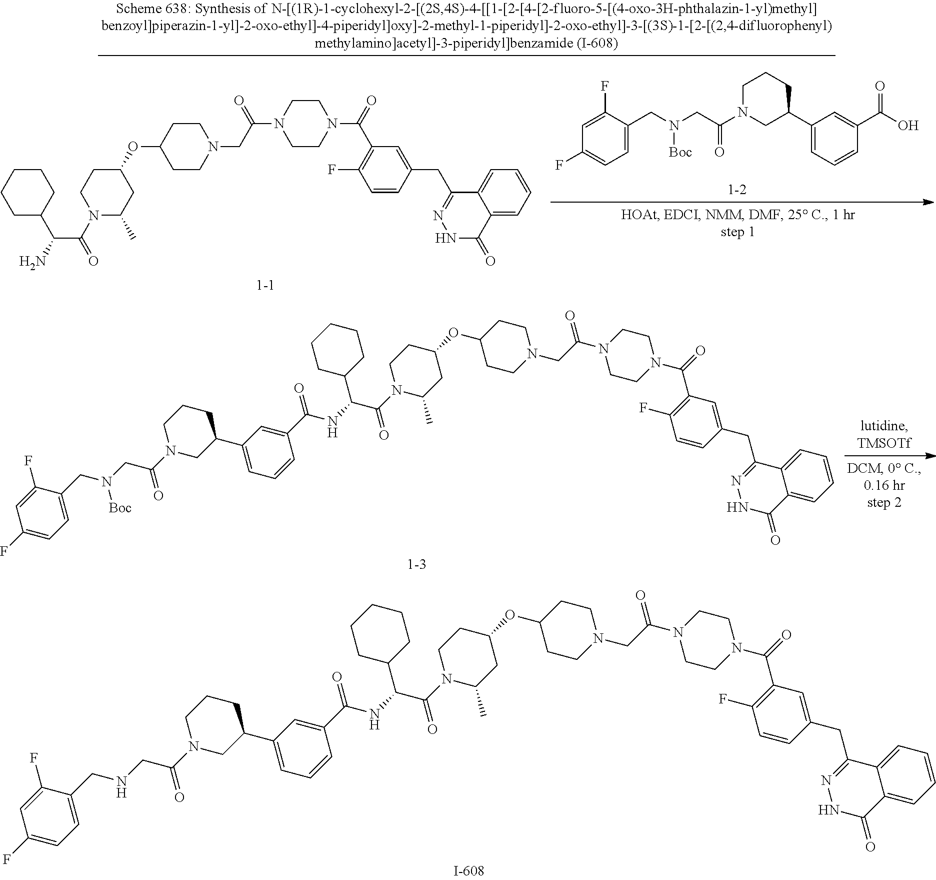

### Step 1: Synthesis of Intermediate 1-3

To a solution of Intermediate 1-2 (40 mg, 81.88 μmol, 1 eq) in DMF (0.5 mL) was added EDCI (47.09 mg, 245.64 μmol, 3 eq), HOAt (11.14 mg, 81.88 μmol, 11.45 μL, 1 eq) and NMM (41.41 mg, 409.40 μmol, 45.01 μL, 5 eq). Then Intermediate 1-1 (63.90 mg, 81.88 μmol, 1 eq, HCl salt) was added. The mixture was stirred at 25° C. for 1 hr. The mixture was washed with water (2 mL) and extracted with EA (2 mL*3). The combined organic layers were dried over sodium sulfate, filtered and concentrated under reduced pressure to give a residue. The residue was purified by flash silica gel chromatography (ISCO; 4 g SepaFlash Silica Flash Column, Eluent of 0˜15% MeOH/EA @25 mL/min) and then the eluent was concentrated in vacuo to give product. Intermediate 1-3 (100 mg, 80.70 μmol, 98.56% yield, 98% purity) was obtained as a yellow solid. LCMS (Method D): Rt=0.480 min, [M+H]⁺=1214.9. SFC: Rt=2.086 min. ¹H NMR (400 MHz, METHANOL-d₄) δ=8.37 (d, J=7.6 Hz, 1H), 7.95 (d, J=7.6 Hz, 1H), 7.89-7.81 (m, 2H), 7.76 (s, 1H), 7.71 (d, J=7.2 Hz, 1H), 7.48 (d, J=7.2 Hz, 2H), 7.42-7.40 (m, 3H), 7.16-7.14 (m, 1H), 6.95 (d, J=7.2 Hz, 2H), 4.96 (d, J=10.0 Hz, 1H), 4.83 (d, J=16.8 Hz, 1H), 4.55 (d, J=12.4 Hz, 2H), 4.47-4.43 (m, 1H), 4.39 (s, 2H), 4.35-4.13 (m, 2H), 4.06-3.97 (m, 1H), 3.90 (d, J=2.0 Hz, 1H), 3.82 (d, J=13.2 Hz, 2H), 3.76-3.70 (m, 2H), 3.67 (s, 1H), 3.54-3.53 (m, 4H), 3.34 (s, 2H), 3.28-3.07 (m, 3H), 2.77 (d, J=7.6 Hz, 4H), 2.46-2.33 (m, 2H), 2.06 (s, 1H), 1.87 (d, J=12.4 Hz, 6H), 1.81-1.74 (m, 4H), 1.72-1.64 (m, 4H), 1.63-1.56 (m, 2H), 1.50 (d, J=5.2 Hz, 2H), 1.47-1.40 (m, 10H), 1.36-1.32 (m, 2H), 1.30 (d, J=7.6 Hz, 2H), 1.16-1.08 (m, 2H). ¹⁹F NMR (376 MHz, METHANOL-d₄) δ=−113.397, −115.689, −120.767.

### Step 2: Synthesis of I-608

To a solution of Intermediate 1-3 (60 mg, 49.41 μmol, 1 eq) in DCM (0.6 mL) was added lutidine (1.59 mg, 14.82 μmol, 1.73 μL, 0.3 eq) and TMSOTf (16.47 mg, 74.11 μmol, 13.39 μL, 1.5 eq). The mixture was stirred at 0° C. for 10 min. The pH was basified to 7-8 with NH₃·H₂O and the mixture was concentrated in vacuo. The residue was purified by reversed phase chromatography (0.1% NH₃·H₂O condition), the eluent was concentrated under reduced pressure to remove ACN and lyophilized to give product. After lyophilization, the residue was further purified by reversed phase chromatography (0.1% FA condition), and the eluent was concentrated under reduced pressure to remove ACN and lyophilized to give product. I-608 (24.81 mg, 21.26 μmol, 43.03% yield, 99.422% purity, FA) was obtained as a white solid. LCMS (Method D): Rt=0.369 min, [M+H]⁺=1114.3. SFC: Rt=2.711 min. ¹H NMR (400 MHz, METHANOL-d₄) δ=8.46 (s, 1H), 8.37 (d, J=7.6 Hz, 1H), 7.95 (d, J=6.8 Hz, 1H), 7.90-7.81 (m, 2H), 7.77 (s, 1H), 7.71 (d, J=2.8 Hz, 1H), 7.53-7.43 (m, 4H), 7.37-7.35 (m, 1H), 7.17-7.16 (m, 1H), 7.05-6.94 (m, 2H), 5.03-4.93 (m, 1H), 4.82 (s, 1H), 4.57-4.47 (m, 1H), 4.39 (s, 2H), 4.03-3.90 (m, 3H), 3.80 (s, 3H), 3.76 (d, J=2.0 Hz, 2H), 3.65 (d, J=12.8 Hz, 5H), 3.55-3.49 (m, 3H), 3.38-3.34 (m, 1H), 3.24-3.09 (m, 2H), 3.06-2.87 (m, 3H), 2.86-2.57 (m, 4H), 2.09-2.02 (m, 1H), 2.00-1.91 (m, 3H), 1.88 (s, 2H), 1.86 (s, 3H), 1.77 (s, 5H), 1.70 (d, J=5.2 Hz, 3H), 1.63-1.55 (m, 1H), 1.50 (s, 1H), 1.36-1.19 (m, 5H), 1.17-1.08 (m, 2H). ¹⁹F NMR (376 MHz, METHANOL-d₄) δ=−111.705, −115.15, −120.764.

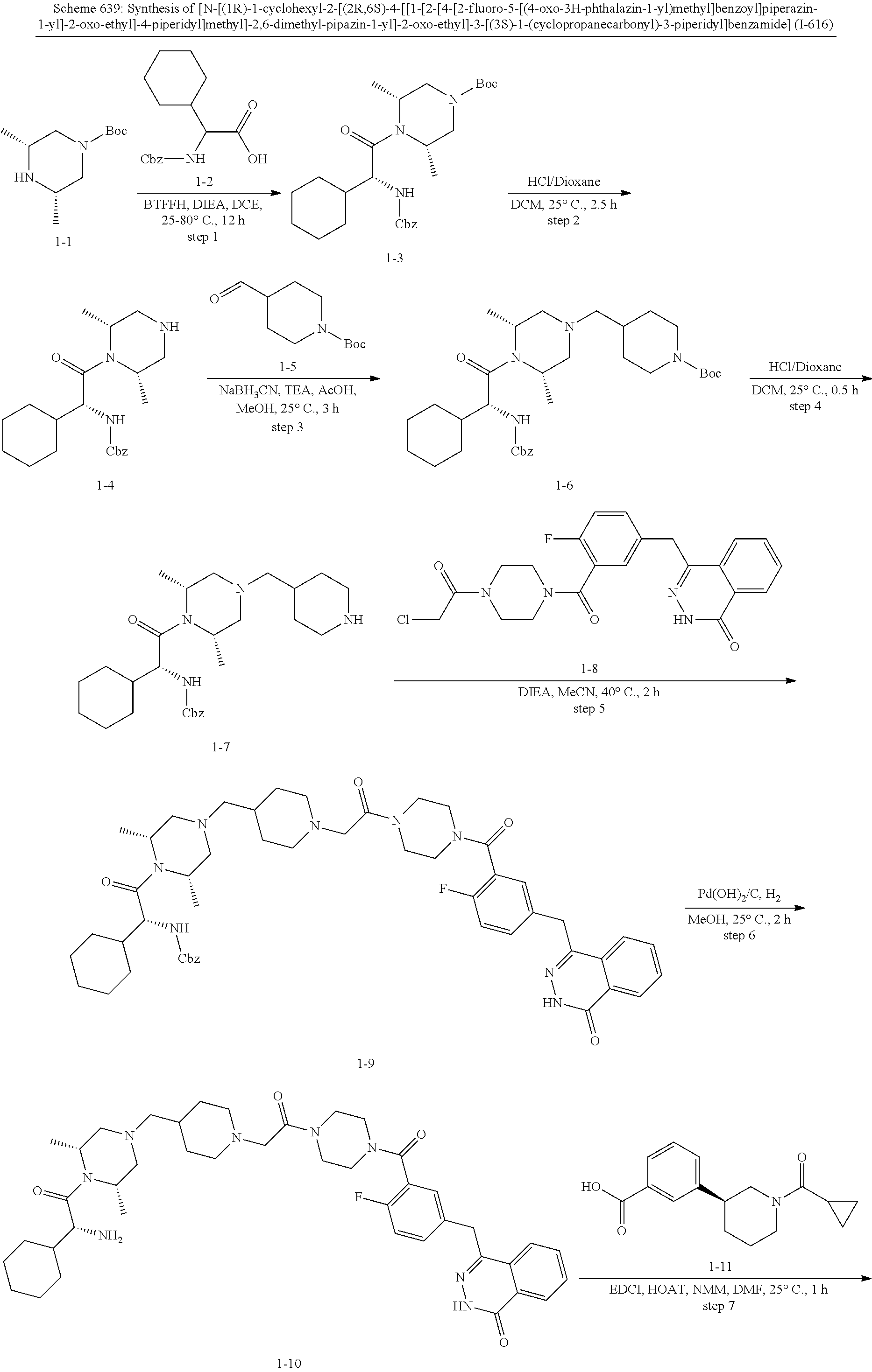

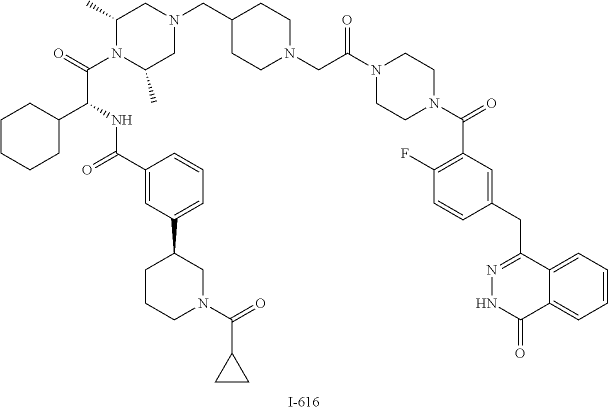 [SMILES: C[C@@H]1CN(CC2CCN(CC(=O)N3CCN(C(=O)c4cc(Cc5n[nH]c(=O)c6ccccc56)ccc4F)CC3)CC2)C[C@H](C)N1C(=O)[C@H](NC(=O)c1cccc([C@@H]2CCCN(C(=O)C3CC3)C2)c1)C1CCCCC1]

### Step 1: Synthesis of Intermediate 1-3

To a mixture of Intermediate 1-2 (1.36 g, 4.67 mmol, 2 eq) and 1-[fluoro(pyrrolidin-1-ium-1-ylidene)methyl]pyrrolidine;hexafluorophosphate (2.95 g, 9.33 mmol, 4 eq) in DCE (5 mL) under N₂, was added DIEA (1.51 g, 11.67 mmol, 2.03 mL, 5 eq), and the mixture was stirred under N₂ at 25° C. for 30 min. Intermediate 1-1 (0.5 g, 2.33 mmol, 1 eq) was added and the mixture was heated and stirred at 80° C. for 12 h. The reaction mixture was diluted with water (10 mL) and extracted with EA (10 mL*3). The combined organic layers were dried over Na₂SO₄, filtered and concentrated under reduced pressure to give a residue. The residue was purified by reversed-phase chromatography (0.1% FA condition). The eluent was concentrated under reduced pressure to give a residue. Intermediate 1-3 (710 mg, 1.46 mmol, 62.41% yield) was obtained as a yellow oil. LCMS (Method D): Retention time: 0.507 min, [M+H]⁺=488.6. SFC: Retention time: 0.783 min, 1.061 min. ¹H NMR (400 MHz, METHANOL-d₄) δ=7.38-7.26 (m, 5H), 5.08 (s, 2H), 4.71-4.39 (m, 2H), 4.29-4.14 (m, 1H), 4.05-3.84 (m, 2H), 3.13-2.81 (m, 2H), 1.82-1.58 (m, 6H), 1.49 (s, 9H), 1.36-1.07 (m, 11H).

### Step 2: Synthesis of Intermediate 1-4

A solution of Intermediate 1-3 (600 mg, 1.23 mmol, 1 eq) in HCl/dioxane (2 M, 6 mL, 9.75 eq) was stirred at 25° C. for 2 hr. The reaction mixture was concentrated under reduced pressure to give a residue. The residue was used for the next step without further purification. Intermediate 1-4 (600 mg, crude) was obtained as a yellow solid. LCMS (Method D): Retention time: 0.328 min, [M+H]⁺=388.2.

### Step 3: Synthesis of Intermediate 1-6

To a solution of Intermediate 1-4 (600 mg, 1.42 mmol, 1 eq, HCl salt) in MeOH (6 mL) was added TEA (859.20 mg, 8.49 mmol, 1.18 mL, 6 eq), and the mixture was stirred at 25° C. for 10 min. AcOH (509.90 mg, 8.49 mmol, 486.09 μL, 6 eq) and Intermediate 1-5 (362.18 mg, 1.70 mmol, 1.2 eq) were added and the mixture was stirred at 25° C. for 30 min, then NaBH₃CN (355.73 mg, 5.66 mmol, 4 eq) was added. The mixture was stirred at 25° C. for 1 h. The reaction mixture was diluted with water (10 mL) and extracted with DCM (10 mL*3). The combined organic layers were dried over Na₂SO₄, filtered and concentrated under reduced pressure to give a residue. The residue was purified by reversed-phase chromatography (0.1% FA condition). The eluent was concentrated under reduced pressure to give a residue. Intermediate 1-6 (260 mg, 423.02 μmol, 29.89% yield, 95.145% purity) was obtained as a white solid. LCMS (Method D): Retention time: 0.356 min, [M+H]⁺=585.9. SFC: Retention time: 2.192 min, 2.622 min.

### Step 4: Synthesis of Intermediate 1-7

A solution of Intermediate 1-6 (250 mg, 427.51 μmol, 1 eq) in HCl/dioxane (2 M, 2.50 mL, 11.70 eq) was stirred at 25° C. for 0.5 hr. The reaction mixture was concentrated under reduced pressure to give a residue. The residue was used for the next step without further purification. Intermediate 1-7 (260 mg, crude, HCl salt) was obtained as a yellow oil. LCMS (Method D): Retention time: 0.295 min, [M+H]⁺=485.3.

### Step 5: Synthesis of Intermediate 1-9

To a solution of Intermediate 1-7 (200 mg, 383.78 μmol, 2 eq, HCl salt) and Intermediate 1-8 (84.98 mg, 191.89 μmol, 1 eq) in ACN (2 mL) was added DIEA (49.60 mg, 383.78 μmol, 66.85 μL, 2 eq). The mixture was stirred at 40° C. for 1.5 hr. The reaction mixture was diluted with water (5 mL) and extracted with DCM (5 mL*3). The combined organic layers were dried over Na₂SO₄, filtered and concentrated under reduced pressure to give a residue. The residue was purified by flash silica gel chromatography (ISCO; 20 g SepaFlash Silica Flash Column, Eluent of 0-100% Petroleum ether/Ethyl acetate @40 mL/min), and the eluent was concentrated under reduced pressure to give a residue. Intermediate 1-9 (200 mg, crude) was obtained as a yellow oil. LCMS (Method D): Retention time: 0.338 min, [M+H]⁺=891.5. SFC: Retention time: 1.465 min, 1.751 min.

### Step 6: Synthesis of Intermediate 1-10

To a solution of Intermediate 1-9 (200 mg, 224.45 μmol, 1 eq) in MeOH (2 mL) was added Pd(OH)₂ (100 mg, 142.41 μmol, 20% purity, 6.35e−1 eq) under N₂ atmosphere. The suspension was degassed and purged with H₂ 3 times. The mixture was stirred under H₂ at 25° C. for 2 hr. The reaction was diluted with MeOH (5 mL*5), filtered and concentrated under reduced pressure to give a residue. The residue was used for the next step without further purification. Intermediate 1-10 (145 mg, 191.56 μmol, 85.35% yield) was obtained as a colorless oil. LCMS (Method D): Retention time: 0.525 min, [M+H]⁺=1074.7.

### Step 7: Synthesis of I-616

To a solution of Intermediate 1-11 (40 mg, 146.35 μmol, 1 eq) in DMF (1 mL) was added HOAt (19.92 mg, 146.35 μmol, 20.47 μL, 1 eq), EDCI (84.16 mg, 439.04 μmol, 3 eq), NMM (74.01 mg, 731.73 μmol, 80.45 μL, 5 eq), and Intermediate 1-10 (110.78 mg, 146.35 μmol, 1 eq). The mixture was stirred at 25° C. for 0.5 hr. The reaction mixture was diluted with water (10 mL) and extracted with DCM (5 mL*3). The combined organic layers were dried over Na₂SO₄, filtered and concentrated under reduced pressure to give a residue. The residue was purified by reversed phase chromatography (0.1% FA condition). The eluent was lyophilized to give product. I-616 (50 mg, 47.25 μmol, 32.28% yield, FA salt) was obtained as a white solid. LCMS (Method D): Retention time: 0.362 min, [M+H]⁺=1012.4. SFC: Retention time: 1.064 min, 1.438 min. ¹H NMR (400 MHz, DMSO-d₆) δ=12.59 (s, 1H), 8.63 (d, J=8.4 Hz, 1H), 8.26 (d, J=7.6 Hz, 1H), 8.23 (s, 1H), 8.00-7.94 (m, 1H), 7.92-7.71 (m, 4H), 7.52-7.33 (m, 4H), 7.23 (t, J=8.8 Hz, 1H), 4.86-4.59 (m, 1H), 4.56-4.37 (m, 2H), 4.37-4.11 (m, 4H), 3.67-3.44 (m, 6H), 3.20-3.05 (m, 5H), 2.84-2.60 (m, 6H), 2.16-1.85 (m, 9H), 1.84-1.55 (m, 10H), 1.38 (s, 3H), 1.26-0.84 (m, 11H), 0.81-0.56 (m, 4H). ¹⁹F NMR (377 MHz, DMSO-d₆) δ=−119.76 (d, J=19.8 Hz, 1F).

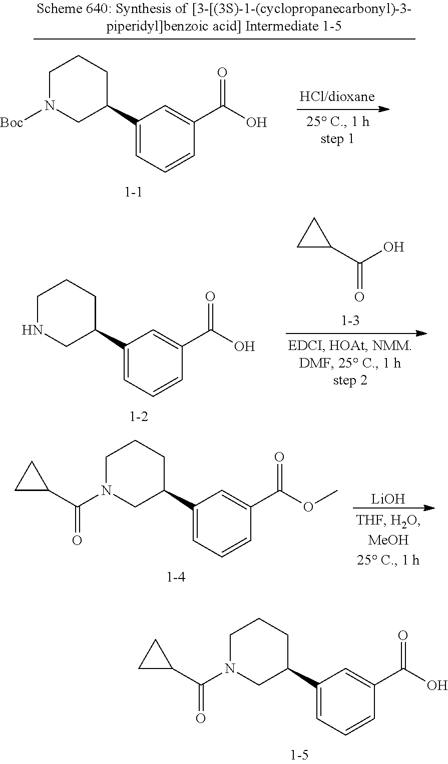

### Step 1: Synthesis of Intermediate 1-2

A solution of Intermediate 1-1 (1 g, 3.13 mmol, 1 eq) in HCl/dioxane (2 M, 10.00 mL, 6.39 eq) was stirred at 25° C. for 1.5 hr. The reaction mixture was concentrated under reduced pressure to give a residue. The residue was used for the next step without further purification. Intermediate 1-2 (710 mg, crude) was obtained as a white solid. LCMS (Method D): Retention time: 0.222 min, [M+H]⁺=220.1.

### Step 2: Synthesis of Intermediate 1-4

To a solution of Intermediate 1-3 (235.64 mg, 2.74 mmol, 216.58 μL, 1 eq) in DMF (7 mL) was added HOAt (372.56 mg, 2.74 mmol, 382.90 μL, 1 eq), EDCI (1.57 g, 8.21 mmol, 3 eq), NMM (1.38 g, 13.69 mmol, 1.50 mL, 5 eq), and Intermediate 1-2 (700 mg, 2.74 mmol, 1 eq, HCl salt). The mixture was stirred at 25° C. for 40 min. The reaction mixture was diluted with water (10 mL) and extracted with DCM (10 mL*3). The combined organic layers were dried over Na₂SO₄, filtered and concentrated under reduced pressure to give a residue. The residue was purified by flash silica gel chromatography (ISCO; 20 g SepaFlash Silica Flash Column, Eluent of 0˜100% Petroleum ether/Ethyl acetate @40 mL/min). The eluent was concentrated under reduced pressure to give Intermediate 1-4 (760 mg, 2.62 mmol, 95.74% yield, 99.077% purity) as a colorless oil. LCMS (Method D): Retention time: 0.370 min, [M+H]⁺=288.2. SFC: Retention time: 1.444 min.

### Step 3: Synthesis of Intermediate 1-5

To a solution of Intermediate 1-4 (750 mg, 2.61 mmol, 1 eq) in MeOH (2.5 mL), THF (2.5 mL), H₂O (2.5 mL) was added LiOH H₂O (219.05 mg, 5.22 mmol, 2 eq). The mixture was stirred at 25° C. for 1 hr. The mixture was adjusted to pH=5 with citric acid and extracted with EA (10 mL*3). The combined organic layers were dried over Na₂SO₄, filtered and concentrated under reduced pressure to give Intermediate 1-5 (900 mg, crude) as a colorless oil. LCMS (Method D): Retention time: 0.353 min, [M+H]⁺=274.1. SFC: Retention time: 1.498 min.

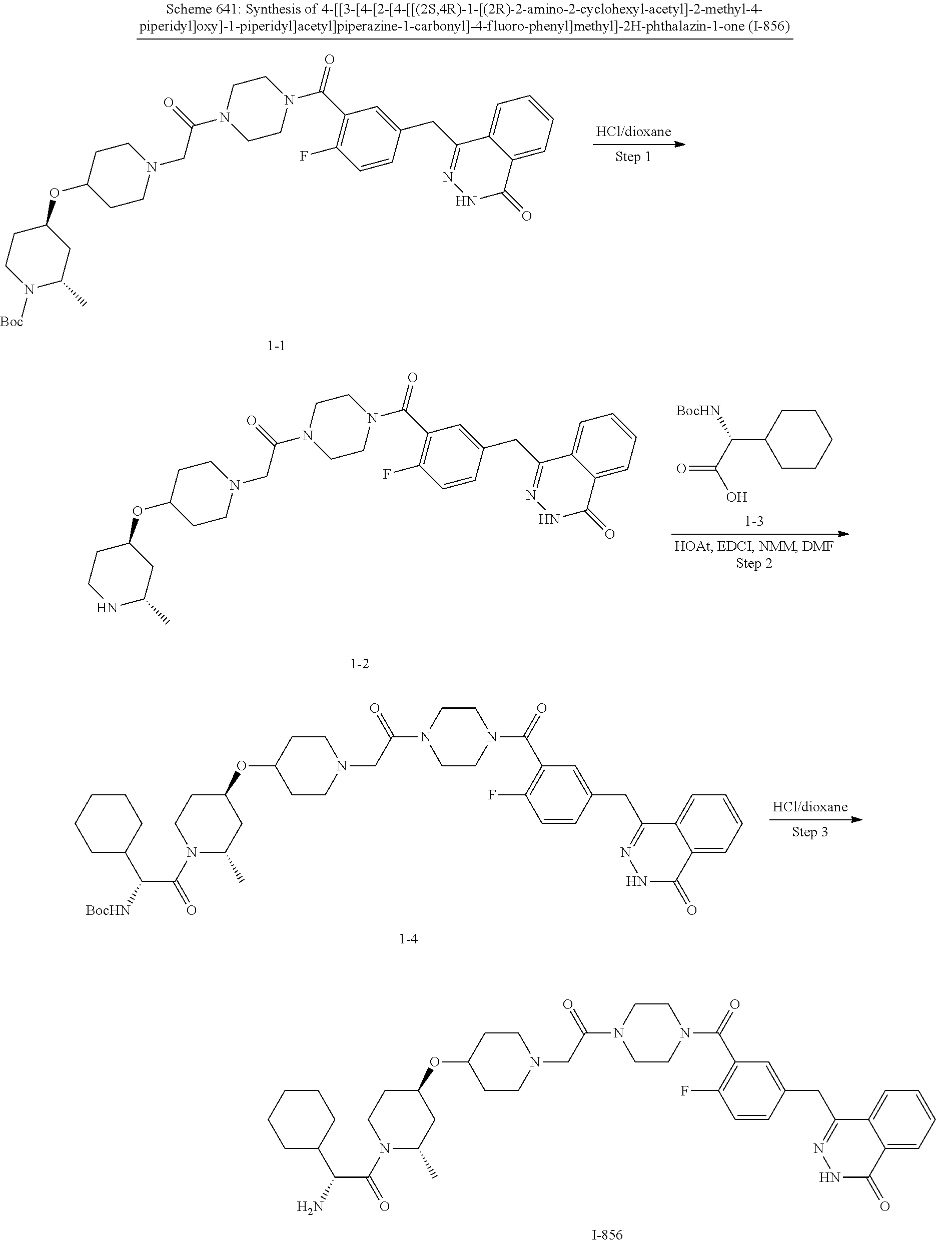 [SMILES: C[C@H]1C[C@H](OC2CCN(CC(=O)N3CCN(C(=O)c4cc(Cc5n[nH]c(=O)c6ccccc56)ccc4F)CC3)CC2)CCN1C(=O)OC(C)(C)C ; C[C@H]1C[C@H](OC2CCN(CC(=O)N3CCN(C(=O)c4cc(Cc5n[nH]c(=O)c6ccccc56)ccc4F)CC3)CC2)CCN1 ; CC(C)(C)OC(=O)N[C@@H](C(=O)O)C1CCCCC1 ; C[C@H]1C[C@H](OC2CCN(CC(=O)N3CCN(C(=O)c4cc(Cc5n[nH]c(=O)c6ccccc56)ccc4F)CC3)CC2)CCN1C(=O)[C@H](NC(=O)OC(C)(C)C)C1CCCCC1 ; C[C@H]1C[C@H](OC2CCN(CC(=O)N3CCN(C(=O)c4cc(Cc5n[nH]c(=O)c6ccccc56)ccc4F)CC3)CC2)CCN1C(=O)[C@H](N)C1CCCCC1]

### Step 1: Synthesis of Intermediate 1-2.

To a mixture of Intermediate 1-1 (40 mg, 56.75 μmol, 1 eq) in DCM (1 mL) was added HCl/dioxane (2 M, 1 mL, 35.24 eq) and the mixture was stirred at 20° C. for 10 min. The reaction mixture was concentrated under reduced pressure to give Intermediate 1-2 (40 mg, crude, HCl salt) as a white solid. LCMS (Method D): Retention time: 0.248 min, [M+H]⁺=605.5.

### Step 2: Synthesis of Intermediate 1-4.

To a mixture of Intermediate 1-2 (30 mg, 46.79 μmol, 1 eq, HCl salt) and Intermediate 1-3 (30.10 mg, 116.97 μmol, 2.5 eq) in DMF (1 mL) was added HOAt (6.37 mg, 46.79 μmol, 6.55 μL, 1 eq), EDCI (26.91 mg, 140.37 μmol, 3 eq), and NMM (23.66 mg, 233.95 μmol, 25.72 μL, 5 eq), then the mixture was stirred at 20° C. for 1 hour. The reaction mixture was diluted with water (20 mL) and extracted with EA (5 mL*3). The combined organic layers were concentrated under reduced pressure to give Intermediate 1-4 (30 mg, 35.54 μmol, 75.97% yield) as a white solid. LCMS (Method D): Retention time: 0.392 min, [M+H]⁺=844.5.

### Step 3: Synthesis of I-856.

To a mixture of Intermediate 1-4 (30 mg, 35.54 μmol, 1 eq) in DCM (0.5 mL) was added HCl/dioxane (2 M, 0.5 mL, 28.13 eq), then the mixture was stirred at 20° C. for 1 hour. The reaction mixture was concentrated under reduced pressure to give a residue. The crude product was purified by reversed-phase chromatography (0.1% FA condition). The eluent was concentrated under reduced pressure to remove MeCN and then lyophilized to give I-856 (10.43 mg, 12.70 μmol, 35.74% yield, 96.2230% purity, FA salt) as a yellow solid. LCMS (Method D): Retention time: 0.284 min, [M+H]⁺=744.8. SFC: Rt=1.796 min. ¹H NMR (400 MHz, METHANOL-d₄) δ=8.44 (s, 1H), 8.37 (d, J=7.6 Hz, 1H), 7.99-7.92 (m, 1H), 7.91-7.80 (m, 2H), 7.50 (s, 1H), 7.37-7.34 (m, 1H), 7.19-7.13 (m, 1H), 5.03-4.96 (m, 1H), 4.58-4.43 (m, 1H), 4.39 (s, 2H), 4.34-4.16 (m, 2H), 3.95-3.74 (m, 4H), 3.67 (d, J=10.4 Hz, 4H), 3.58 (d, J=8.0 Hz, 1H), 3.51 (d, J=18.4 Hz, 2H), 3.09-2.98 (m, 2H), 2.75-2.56 (m, 2H), 2.13-2.06 (m, 1H), 2.02-1.92 (m, 3H), 1.86-1.67 (m, 10H), 1.54-1.46 (m, 1H), 1.34 (d, J=6.8 Hz, 2H), 1.30 (s, 1H), 1.27 (s, 1H), 1.30-1.19 (m, 3H). ¹⁹F NMR (376 MHz, METHANOL-d₄) δ=−120.77.

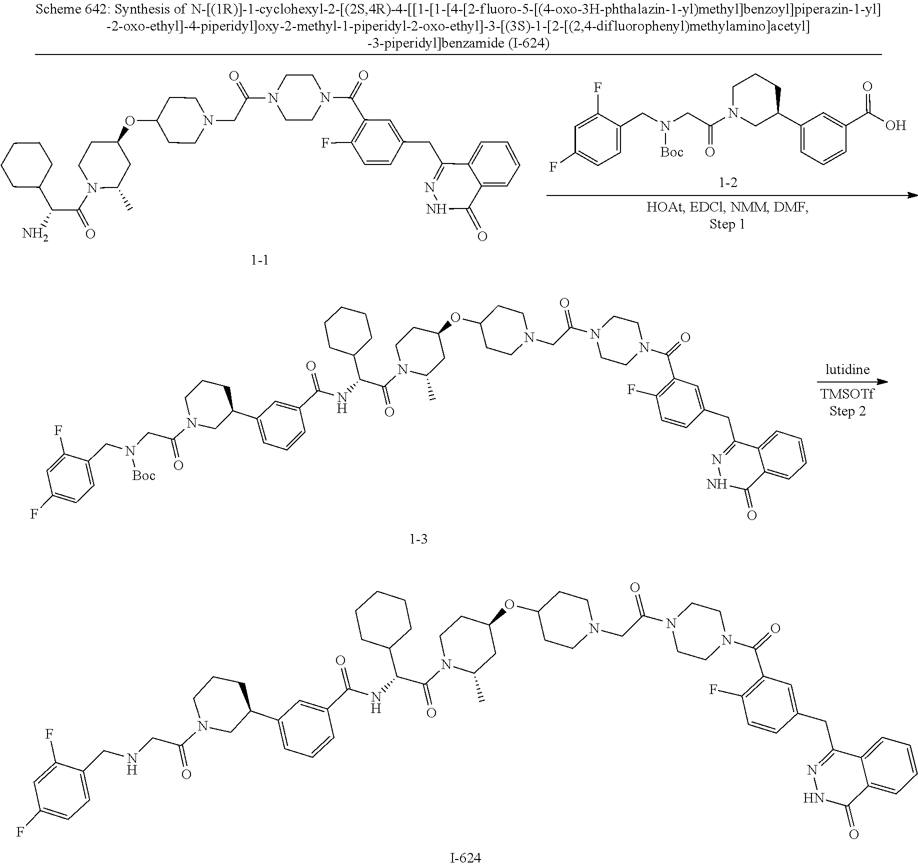

### Step 1: Synthesis of Intermediate 1-2.

To a mixture of Intermediate 1-1 (78 mg, 99.95 μmol, 1 eq, HCl salt) and Intermediate 1-2 (48.83 mg, 99.95 μmol, 1 eq) in DMF (1 mL) was added HOAt (13.60 mg, 99.95 μmol, 13.98 μL, 1 eq), EDCI (57.48 mg, 299.86 μmol, 3 eq) and NMM (50.55 mg, 499.76 μmol, 54.95 μL, 5 eq), then the mixture was stirred at 20° C. for 1 hour. The reaction mixture was diluted with water (50 mL) and extracted with EA (10 mL*3). The combined organic layers were concentrated under reduced pressure to give Intermediate 1-3 (100 mg, crude) as a white solid. LCMS (Method D): Retention time: 0.466 min, [M+H]⁺=1214.2.

### Step 2: Synthesis of I-624.

To a mixture of Intermediate 1-3 (70 mg, 57.64 μmol, 1 eq) in DCM (2 mL) was added 2,6-dimethylpyridine (1.85 mg, 17.29 μmol, 2.01 μL, 0.3 eq) and trimethylsilyl trifluoromethanesulfonate (19.22 mg, 86.46 μmol, 15.62 μL, 1.5 eq), then the mixture was stirred at 0° C. for 10 min. The reaction mixture was concentrated under reduced pressure to give a residue. The crude product was purified by reversed-phase chromatography (0.1% FA condition). The eluent was concentrated under reduced pressure to remove MeCN and then lyophilized to give I-624 (22.61 mg, 18.89 μmol, 32.77% yield, 96.9510% purity, FA salt) as a white solid. LCMS (Method D): Retention time: 0.373 min, [M+H]⁺=1114.3. SFC: Rt=1.736 min. ¹H NMR (400 MHz, METHANOL-d₄) δ=8.59-8.44 (m, 1H), 8.41-8.33 (m, 1H), 7.99-7.92 (m, 1H), 7.91-7.80 (m, 2H), 7.79-7.67 (m, 2H), 7.52-7.41 (m, 4H), 7.37 (s, 1H), 7.19-7.15 (m, 1H), 7.03-6.91 (m, 2H), 5.03-4.91 (m, 2H), 4.66-4.50 (m, 2H), 4.39 (s, 2H), 4.31-4.11 (m, 1H), 3.93-3.50 (m, 14H), 3.42 (d, J=6.8 Hz, 1H), 3.18-3.11 (m, 1H), 2.95-2.66 (m, 5H), 2.51-2.32 (m, 2H), 2.05 (s, 2H), 1.94-1.55 (m, 16H), 1.51-1.42 (m, 1H), 1.38-1.35 (m, 1H), 1.30-1.19 (m, 5H), 1.16-1.06 (m, 2H). ¹⁹F NMR (376 MHz, METHANOL-d₄) δ=−112.55, δ=−115.68, δ=−120.74.

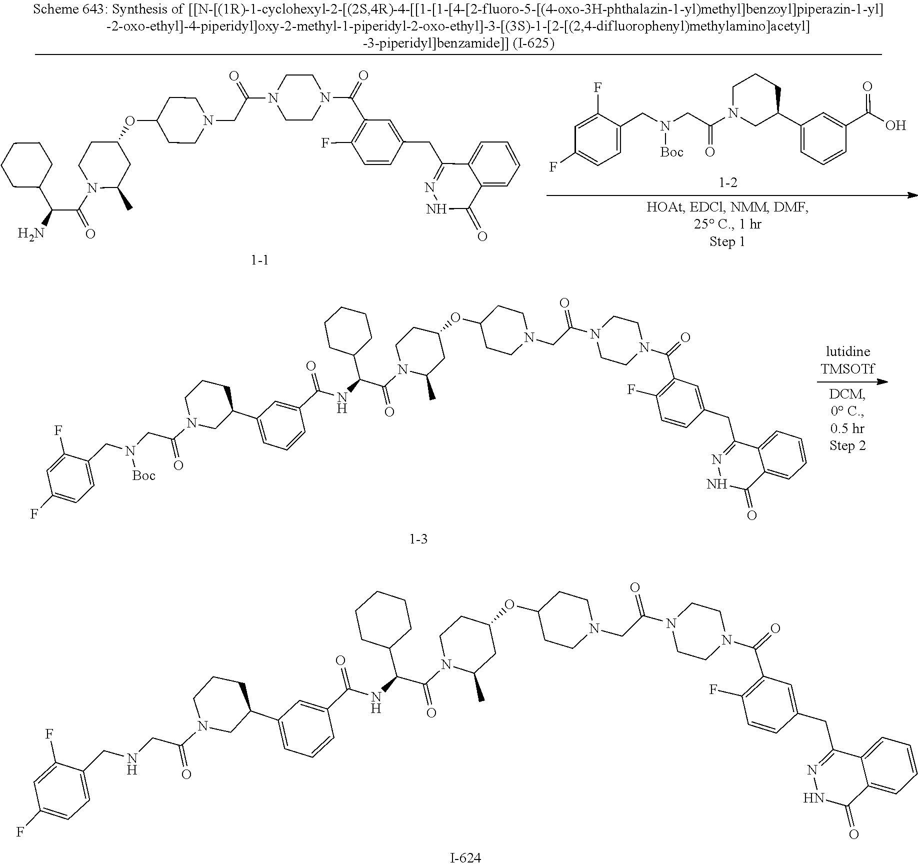

### Step 1: Synthesis of Intermediate 1-3

To a solution of Intermediate 1-2 (46.95 mg, 96.11 μmol, 1 eq) in DMF (1 mL) was added EDCI (55.27 mg, 288.33 μmol, 3 eq), HOAt (13.08 mg, 96.11 μmol, 13.44 μL, 1 eq), Intermediate 1-1 (75 mg, 96.11 μmol, 1 eq, HCl salt) and NMM (48.61 mg, 480.54 μmol, 52.83 μL, 5 eq). The mixture was stirred at 25° C. for 1 hr. The mixture was filtered. The filtrate was purified by reversed-phase HPLC (0.1% FA condition). The eluent was concentrated under reduced pressure to remove ACN and then lyophilized to give Intermediate 1-3 (65 mg, 53.52 μmol, 55.69% yield, 100% purity) as a white solid. LCMS (Method D): Rt: 0.469 min, [M+H]⁺=1214.3.

### Step 2: Synthesis of I-625

To a solution of Intermediate 1-3 (55 mg, 45.29 μmol, 1 eq) in DCM (1 mL) was added 2,6-dimethylpyridine (2.91 mg, 27.17 μmol, 3.16 μL, 0.6 eq) and trimethylsilyl trifluoromethanesulfonate (15.10 mg, 67.93 μmol, 12.28 μL, 1.5 eq). The mixture was stirred at 0° C. for 30 min. To the mixture was added NH₃·H₂O to adjust the pH to 8. The mixture was concentrated under vacuum. The crude product was purified by reversed-phase chromatography (0.1% NH₃·H₂O condition). The eluent was concentrated under reduced pressure to remove ACN and then lyophilized to give a product. The product was further purified by reversed-phase chromatography (0.1% FA condition). The eluent was concentrated under reduced pressure to remove ACN and then lyophilized to give I-625 (19.04 mg, 16.38 μmol, 36.16% yield, 99.8% purity, FA salt) as a white solid. LCMS (Method D): Rt: 0.371 min, [M+H]⁺=1114.3. SFC: Rt: 1.496 min. ¹H NMR (400 MHz, METHANOL-d₄) δ=8.59-8.42 (m, 1H), 8.40-8.33 (m, 1H), 7.99-7.92 (m, 1H), 7.91-7.79 (m, 2H), 7.78-7.66 (m, 2H), 7.54-7.33 (m, 5H), 7.21-7.12 (m, 1H), 7.04-6.90 (m, 2H), 4.94-4.89 (m, 1H), 4.85-4.79 (m, 1H), 4.58-4.48 (m, 1H), 4.39 (s, 2H), 4.27-4.09 (m, 1H), 3.97-3.86 (m, 3H), 3.86-3.44 (m, 11H), 3.41-3.32 (m, 4H), 3.22-3.10 (m, 1H), 2.95-2.80 (m, 3H), 2.79-2.67 (m, 1H), 2.56-2.32 (m, 2H), 2.18-2.00 (m, 2H), 2.00-1.48 (m, 15H), 1.47-1.01 (m, 10H). ¹⁹F NMR (376 MHz, METHANOL-d₄) δ=−112.373, −115.555, −120.727.

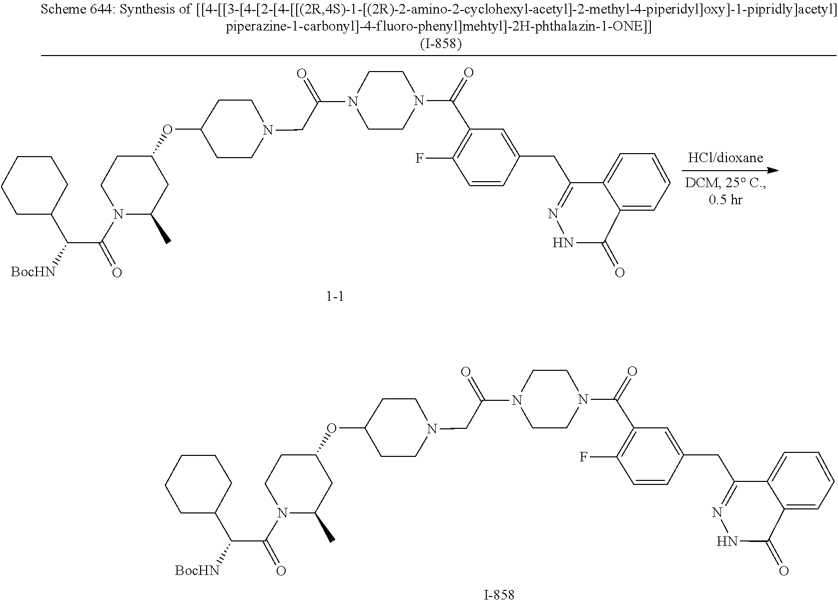 [SMILES: C[C@@H]1C[C@@H](OC2CCN(CC(=O)N3CCN(C(=O)c4cc(Cc5n[nH]c(=O)c6ccccc56)ccc4F)CC3)CC2)CCN1C(=O)[C@H](NC(=O)OC(C)(C)C)C1CCCCC1 ; C[C@@H]1C[C@@H](OC2CCN(CC(=O)N3CCN(C(=O)c4cc(Cc5n[nH]c(=O)c6ccccc56)ccc4F)CC3)CC2)CCN1C(=O)[C@H](NC(=O)OC(C)(C)C)C1CCCCC1]

A solution of Intermediate 1-1 (30 mg, 35.54 μmol, 1 eq) in CH₂Cl₂ (0.3 mL) and HCl/dioxane (2 M, 0.3 mL) was stirred at 25° C. for 1 hr. The mixture was concentrated. The residue was purified by reversed-phase chromatography (0.10% HCl condition). The eluent was concentrated under reduced pressure to remove ACN and then lyophilized to give I-858 (17.31 mg, 22.18 μmol, 62.41% yield, 100% purity, HCl salt) as a white solid. LCMS (Method D): Rt: 0.290 min, [M+H]⁺=744.4. ¹H NMR (400 MHz, METHANOL-d₄) δ=8.36 (d, J=7.6 Hz, 1H), 8.00-7.93 (m, 1H), 7.92-7.79 (m, 2H), 7.54-7.46 (m, 1H), 7.42-7.35 (m, 1H), 7.20-7.13 (m, 1H), 4.89-4.83 (m, 1H), 4.41-4.27 (m, 5H), 4.05-3.60 (m, 7H), 3.59-3.51 (m, 2H), 3.50-3.32 (m, 6H), 3.27-3.13 (m, 1H), 2.25-2.15 (m, 1H), 2.12-1.93 (m, 4H), 1.90-1.62 (m, 7H), 1.59-1.42 (m, 1H), 1.40-1.14 (m, 9H). SFC: Rt=2.056 min. ¹⁹F NMR (376 MHz, METHANOL-d₄) δ=−120.719.

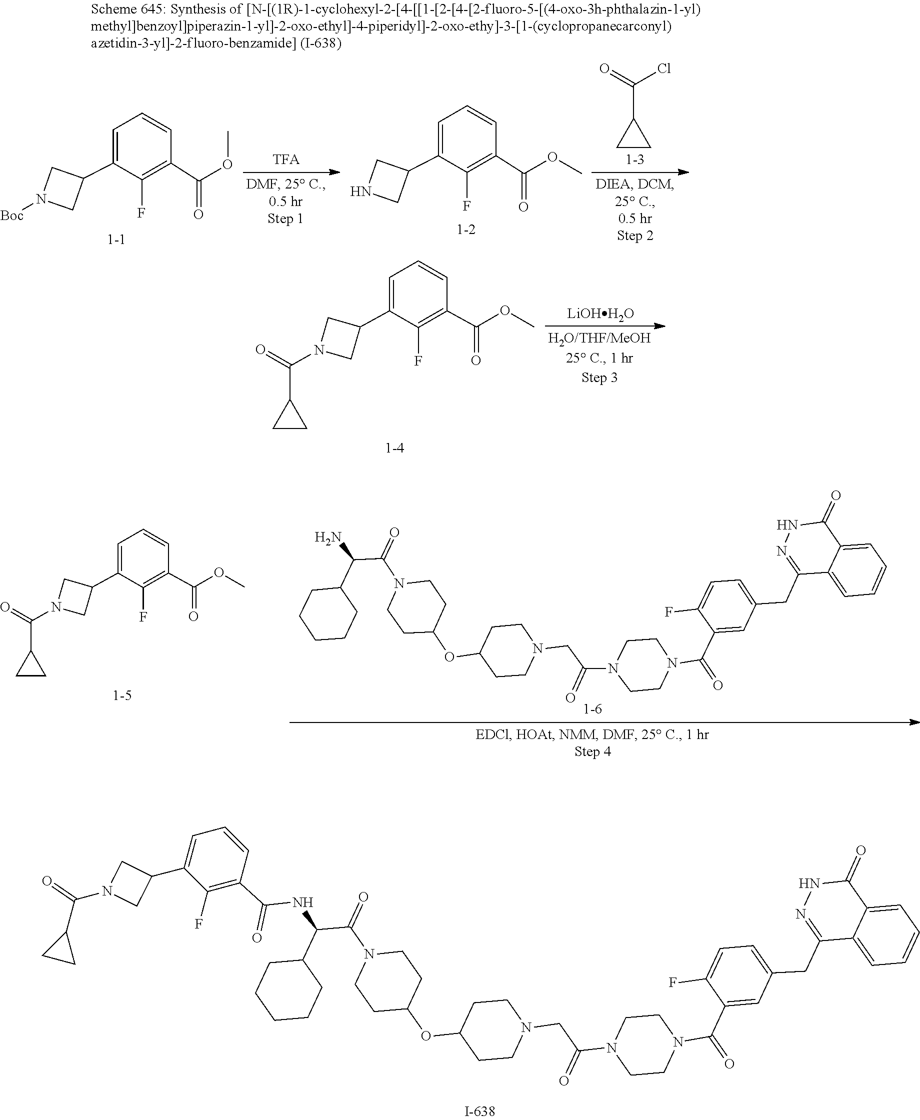

### Step 1: Synthesis of Intermediate 1-2.

To a solution of Intermediate 1-1 (290 mg, 937.50 μmol, 1 eq) in DCM (2.5 mL) was added TFA (767.50 mg, 6.73 mmol, 0.5 mL). The mixture was stirred at 25° C. for 0.5 hr. The mixture was concentrated under reduced pressure to give Intermediate 1-2 (300 mg, crude, TFA salt) as a yellow oil. LCMS (Method D): Rt=0.147 min, [M+H]⁺=210.0.

### Step 2: Synthesis of Intermediate 1-4.

To a solution of Intermediate 1-2 (140 mg, 433.12 μmol, 1 eq, TFA salt) and Intermediate 1-3 (54.33 mg, 519.74 μmol, 47.16 μL, 1.2 eq) in DCM (1.5 mL) was added DIEA (83.97 mg, 649.67 μmol, 113.16 μL, 1.5 eq). The mixture was stirred at 25° C. for 1 hr. The mixture was quenched by addition of H₂O (1.5 mL) at 25° C., and then the mixture was extracted with DCM (1 mL*3), the combined organic phase was dried over Na₂SO₄, filtered and concentrated under reduced pressure to give a crude product. The crude product was purified by reversed-phase chromatography (0.1% FA condition), the eluent was concentrated to remove ACN and lyophilized to give Intermediate 1-4 (85 mg, 273.50 μmol, 63.15% yield, 89.223% purity) as a white solid. LCMS (Method D): Rt=0.390 min, [M+H]⁺=278.1.

### Step 3: Synthesis of Intermediate 1-5.

To a solution of Intermediate 1-4 (30 mg, 108.19 μmol, 1 eq) in H₂O (0.1 mL), THF (0.1 mL) and MeOH (0.1 mL) was added LiOH·H₂O (13.62 mg, 324.57 μmol, 3 eq). The mixture was stirred at 25° C. for 0.5 hr. The pH of the mixture was adjusted to 2-3 with 1M HCl and the mixture was concentrated under reduced pressure to give Intermediate 1-5 (30 mg, crude) as a white solid. LCMS (Method D): Rt=0.303 min, [M+H]⁺=264.1.

### Step 4: Synthesis of Intermediate 1-7.

To a solution of Intermediate 1-5 (30 mg, 113.95 μmol, 1 eq) and Intermediate 1-6 (87.33 mg, 113.95 μmol, 1 eq, HCl salt) in DMF (0.3 mL) was added EDCI (65.54 mg, 341.86 μmol, 3 eq), HOAt (15.51 mg, 113.95 μmol, 1 eq) and NMM (57.63 mg, 569.77 μmol, 62.64 μL, 5 eq). The mixture was stirred at 25° C. for 1 hr. The mixture was quenched by addition of H₂O (1 mL) at 25° C., and extracted with DCM (1 mL*3), the combined organic phase was dried over Na₂SO₄, filtered and concentrated under reduced pressure to give a crude product. The crude product was purified by reversed-phase chromatography (0.1% FA condition), and the eluent was concentrated to remove ACN and lyophilized to give I-638 (19.79 mg, 19.00 μmol, 16.67% yield, 98.043% purity, FA salt) as a white solid. LCMS (Method D): Rt=0.394 min, [M+H]⁺=975.3. SFC: Rt=1.495 min, 2.377 min. ¹H NMR (400 MHz, METHANOL-d₄) δ=8.50 (s, 1H), 8.37 (d, J=7.6 Hz, 1H), 7.98-7.92 (m, 1H), 7.91-7.80 (m, 2H), 7.60 (t, J=7.2 Hz, 2H), 7.49 (s, 1H), 7.38 (d, J=1.6 Hz, 1H), 7.30 (t, J=8.0 Hz, 1H), 7.16 (t, J=8.8 Hz, 1H), 4.96 (d, J=7.6 Hz, 1H), 4.77 (t, J=8.8 Hz, 2H), 4.50-4.36 (m, 4H), 4.26-4.09 (m, 2H), 4.07-3.98 (m, 1H), 3.96-3.87 (m, 1H), 3.85 (d, J=9.2 Hz, 1H), 3.82-3.65 (m, 5H), 3.63-3.37 (m, 6H), 3.23 (s, 1H), 2.84 (d, J=11.6 Hz, 2H), 2.50-2.28 (m, 2H), 1.97-1.73 (m, 8H), 1.73-1.57 (m, 6H), 1.55-1.46 (m, 1H), 1.32-1.09 (m, 5H), 0.92-0.79 (m, 4H). ¹⁹F NMR (376 MHz, METHANOL-d₄) δ=−120.494-−121.779 (m, 1F).

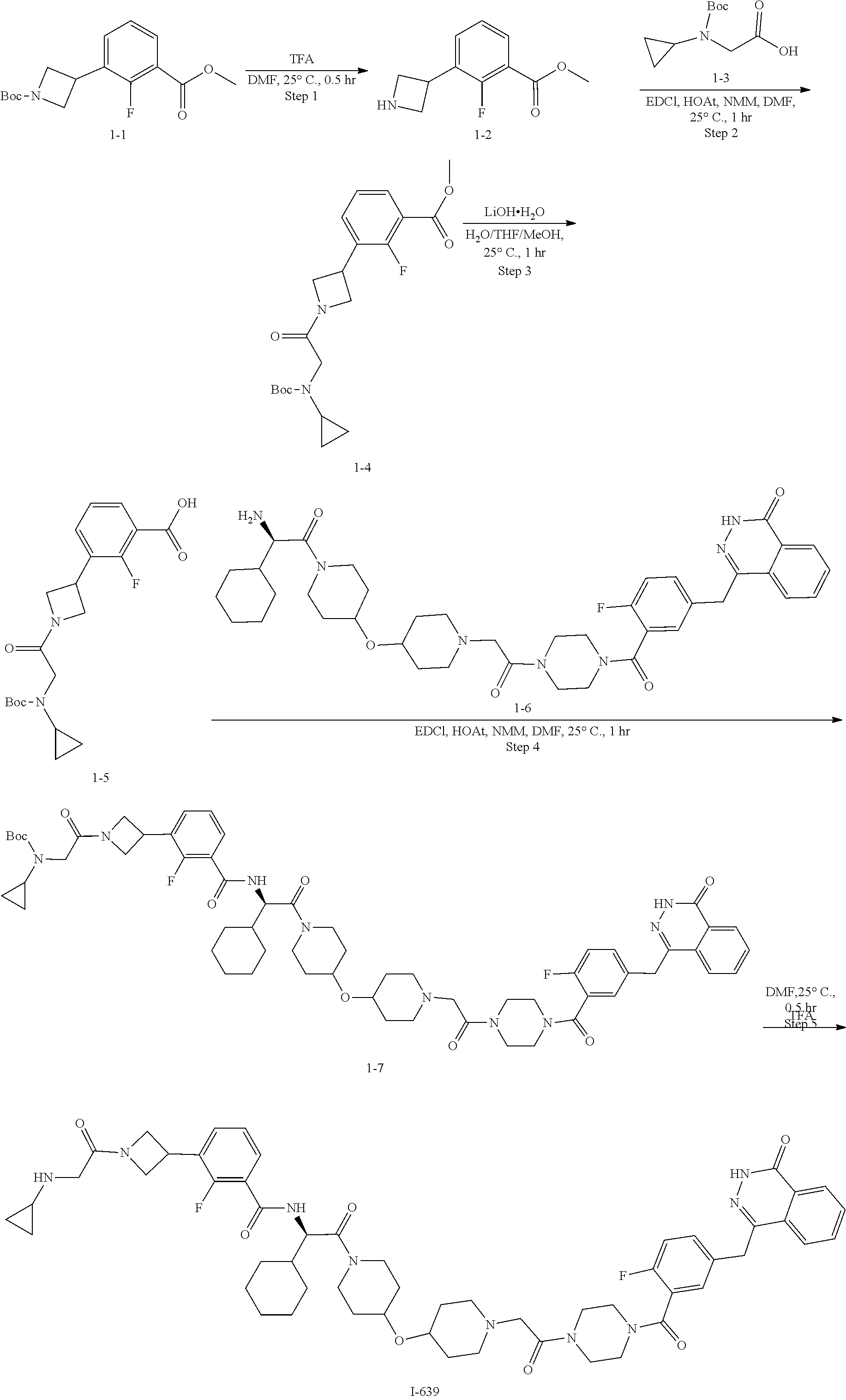

### Step 1: Synthesis of Intermediate 1-2.

To a solution of Intermediate 1-1 (290 mg, 937.50 μmol, 1 eq) in DCM (2.5 mL) was added TFA (767.50 mg, 6.73 mmol, 0.5 mL). The mixture was stirred at 25° C. for 0.5 hr. The mixture was concentrated under reduced pressure to give a crude product Intermediate 1-2 (300 mg, crude, TFA salt) as a yellow oil, which was used for the next step directly. LCMS (Method D): Rt=0.147 min, [M+H]⁺=210.0.

### Step 2: Synthesis of Intermediate 1-4.

To a solution of Intermediate 1-2 (140 mg, 433.12 μmol, 1 eq, TFA salt) and Intermediate 1-3 (111.87 mg, 519.74 μmol, 1.2 eq) in DMF (1.5 mL) was added EDCI (249.09 mg, 1.30 mmol, 3 eq), HOAt (58.95 mg, 433.12 μmol, 1 eq) and NMM (219.04 mg, 2.17 mmol, 238.09 μL, 5 eq). The mixture was stirred at 25° C. for 1 hr. The mixture was quenched by addition of H₂O (2 mL) at 25° C., and the mixture was extracted with DCM (2 mL*3), the combined organic phase was dried over Na₂SO₄, filtered and concentrated under reduced pressure to give a crude product. The crude product was purified by reversed-phase chromatography (0.1% FA condition), the eluent was concentrated to remove ACN and lyophilized to give Intermediate 1-4 (110 mg, 266.11 μmol, 61.44% yield, 98.327% purity) as a yellow oil. LCMS (Method D): Rt=0.382 min, [M+H]⁺=307.1.

### Step 3: Synthesis of Intermediate 1-5.

To a solution of Intermediate 1-4 (100 mg, 246.03 μmol, 1 eq) in H₂O (0.3 mL), THF (0.3 mL), and MeOH (0.3 mL) was added LiOH·H₂O (30.97 mg, 738.10 μmol, 3 eq). The mixture was stirred at 25° C. for 1 hr. The pH of the mixture was adjusted to 2-3 with 1M HCl, extracted with DCM (1 mL*3), and the combined organic phase was dried over Na₂SO₄, filtered and concentrated under reduced pressure to give Intermediate 1-5 (100 mg, crude) as a white solid. LCMS (Method D): Rt=0.378 min, [M+H]⁺=293.0.

### Step 4: Synthesis of Intermediate 1-7.

To a solution of Intermediate 1-5 (195.29 mg, 254.83 μmol, 1 eq, HCl salt) in DMF (1 mL) was added EDCI (146.55 mg, 764.49 μmol, 3 eq), HOAt (34.69 mg, 254.83 μmol, 1 eq) and NMM (128.88 mg, 1.27 mmol, 140.08 μL, 5 eq). The mixture was stirred at 25° C. for 1 hr. The mixture was quenched by addition of H₂O (2 mL) at 25° C., extracted with DCM (2 mL*3), the combined organic phase was dried over Na₂SO₄, filtered and concentrated under reduced pressure to give a crude product. The crude product was purified by reversed-phase chromatography (0.1% FA condition), the eluent was concentrated to remove ACN and lyophilized to give Intermediate 1-7 (130 mg, 106.13 μmol, 41.65% yield, 90.156% purity) as a yellow solid. LCMS (Method D): Rt=0.397 min, [M+H]⁺=1104.7.

### Step 5: Synthesis of I-639.

To a solution of Intermediate 1-7 (120 mg, 108.67 μmol, 1 eq) in DCM (1 mL) was added TFA (307.00 mg, 2.69 mmol, 0.2 mL). The mixture was stirred at 25° C. for 0.5 hr. The mixture was dried with a stream of N₂. 10 mL of H₂O was added and the mixture was lyophilized to give I-639 (107.34 mg, 91.03 μmol, 83.77% yield, 94.831% purity, TFA salt) as a yellow solid. LCMS (Method D): Rt=0.334 min, [M+H]⁺=1004.3. SFC: Rt=1.956 min, 3.538 min. ¹H NMR (400 MHz, METHANOL-d₄) δ=8.36 (d, J=7.6 Hz, 1H), 7.98-7.92 (m, 1H), 7.90-7.80 (m, 2H), 7.62 (q, J=7.6 Hz, 2H), 7.54-7.47 (m, 1H), 7.37 (t, J=8.0 Hz, 1H), 7.34-7.27 (m, 1H), 7.17 (t, J=8.8 Hz, 1H), 4.97 (d, J=7.6 Hz, 1H), 4.71-4.64 (m, 1H), 4.51 (t, J=8.8 Hz, 1H), 4.45-4.36 (m, 3H), 4.34-4.19 (m, 4H), 4.07-3.91 (m, 4H), 3.85-3.68 (m, 5H), 3.65 (s, 1H), 3.59-3.34 (m, 8H), 3.23-3.07 (m, 1H), 2.86-2.75 (m, 1H), 2.26-2.01 (m, 4H), 2.01-1.93 (m, 1H), 1.93-1.74 (m, 6H), 1.70 (d, J=9.6 Hz, 3H), 1.61-1.51 (m, 1H), 1.36-1.06 (m, 6H), 0.96-0.86 (m, 4H). ¹⁹F NMR (376 MHz, METHANOL-d₄) δ=−77.25 (s, 4F), −120.05-−121.12 (m, 1F).

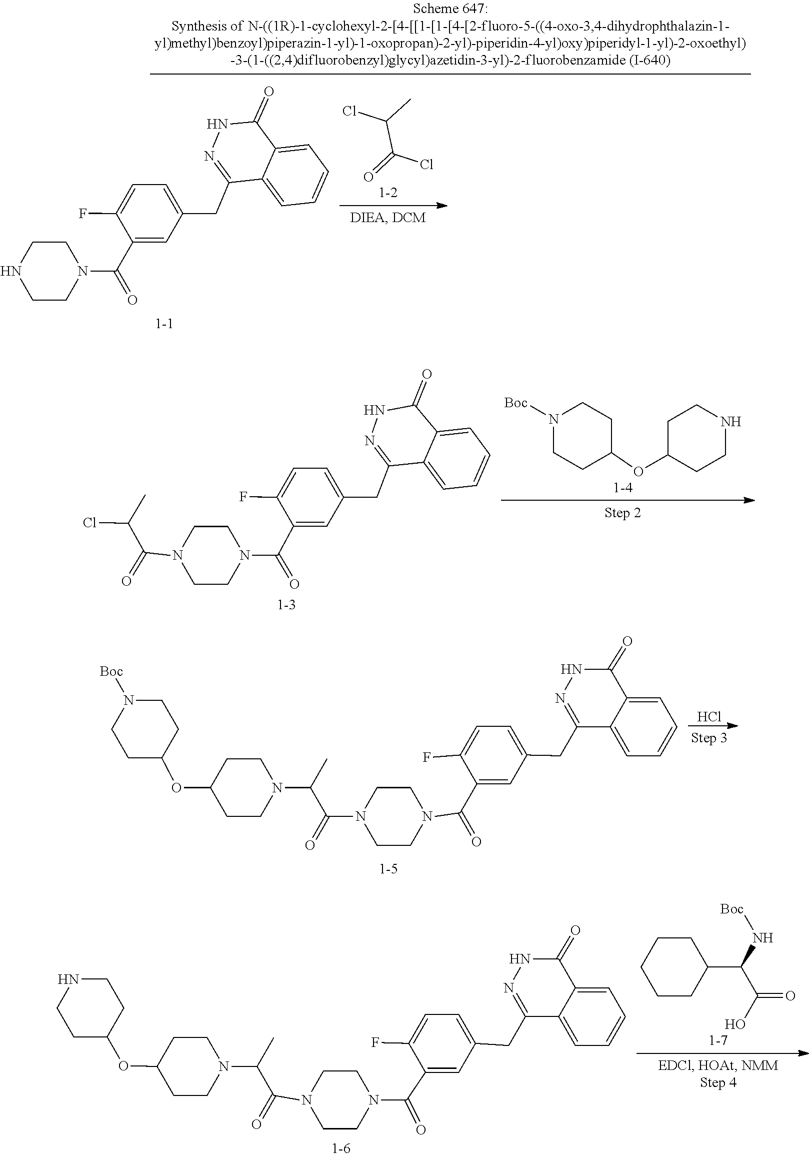

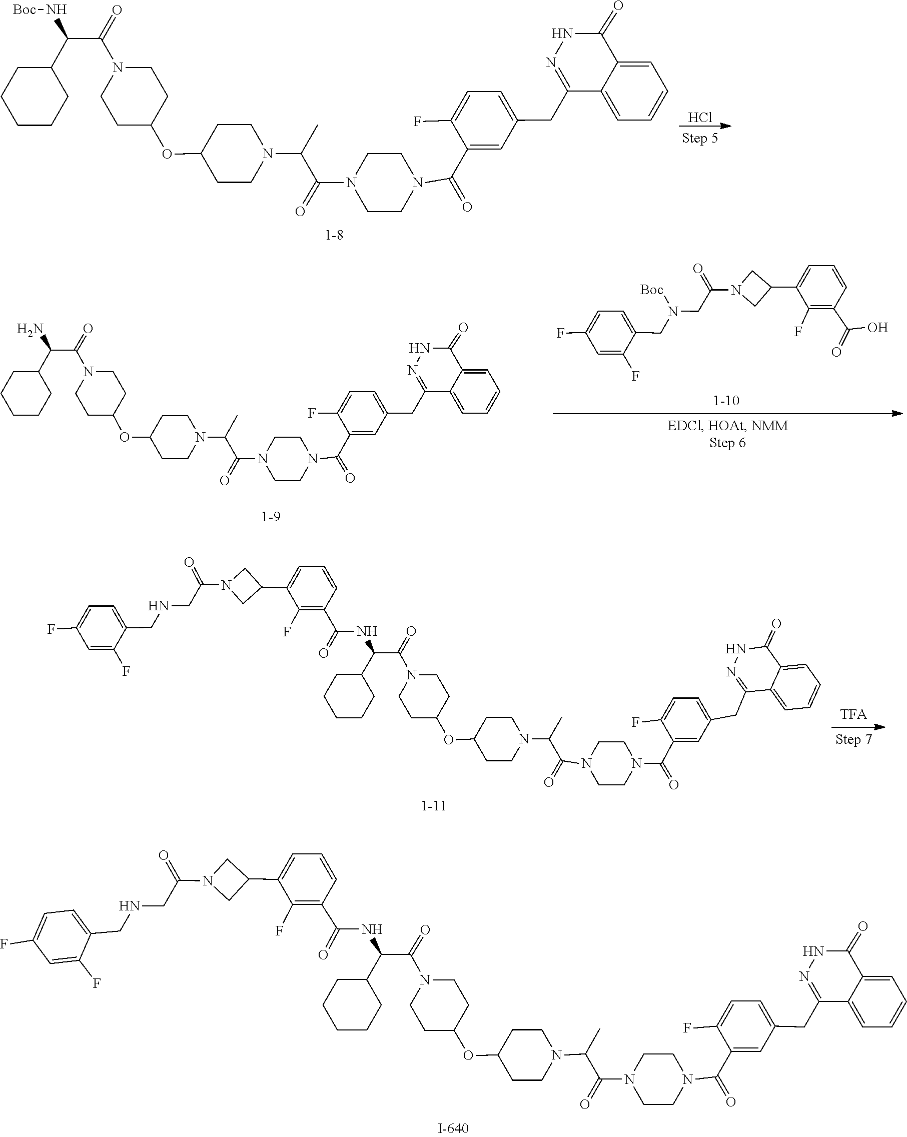

### Step 1: Synthesis of Intermediate 1-3

To the solution of Intermediate 1-1 (0.2 g, 545.87 gmol, 1 eq) in DCM (2 mL) was added Intermediate 1-2 (103.96 mg, 818.80 gmol, 79.48 μL, 1.5 eq) and DIEA (211.64 mg, 1.64 mmol, 285.24 L, 3 eq), and the mixture was stirred at 25° C. for 1 hr. The reaction mixture was concentrated under vacuum. The residue was purified by flash silica gel chromatography (ISCO; 20 g SepaFlash Silica Flash Column, Eluent of 0˜100% Ethyl acetate/Petroleum ethergradient @40 mL/min). Intermediate 1-3 (220 mg, 481.51 μmol, 88.21% yield) was obtained as a white solid. LCMS (Method G): Rt=0.517 min, [M+H]+=457.2.

### Step 2: Synthesis of Intermediate 1-5

To the solution of Intermediate 1-3 (220 mg, 481.51 μmol, 1 eq) in DCM (2 mL) was added Intermediate 1-4 (164.33 mg, 577.81 μmol, 1.2 eq) and DIEA (186.69 mg, 1.44 mmol, 251.61 μL, 3 eq), and the mixture was stirred at 60° C. for 12 hrs. The mixture was diluted with water (5 mL) and extracted with EtOAc (5 mL*3). The organic layer was concentrated under vacuum. The residue was purified by Pre-HPLC (column: Waters Xbridge Prep OBD C18 150*40 mm*10 um; mobile phase: [water(NH₄ICO₃)-ACN]; gradient: 30%-60% B over 52 min) and dried by lyophilization. Intermediate 1-5 (270 mg, 383.07 μmol, 79.56% yield) was obtained as a gray solid. LCMS (Method G): Rt=0.630 min, [M+H]+=705.4.

### Step 3: Synthesis of Intermediate 1-6

A mixture of Intermediate 1-5 (210 mg, 297.94 μmol, 1 eq) in DCM (1 mL) was added HCl/dioxane (2 M, 2 mL, 13.43 eq) and the mixture was stirred at 25° C. for 0.5 hr. The mixture was concentrated under vacuum. Intermediate 1-6 (0.2 g, crude, HCl salt) was obtained as a brown solid. LCMS (Method G): Rt=0.678 min, [M+H]+=605.4. ¹H NMR (400 MHz, DMSO-d6) δ=12.71-12.53 (m, 1H), 10.23-9.85 (m, 1H), 8.26 (d, J=7.8 Hz, 1H), 8.00-7.94 (m, 1H), 7.93-7.79 (m, 2H), 7.46 (br s, 1H), 7.37 (br t, J=5.3 Hz, 1H), 7.25 (br t, J=8.9 Hz, 1H), 5.75 (s, 2H), 4.76 (br d, J=4.9 Hz, 3H), 3.90-3.69 (m, 4H), 3.45-3.33 (m, 3H), 3.26 (br d, J=2.1 Hz, 3H), 3.20-3.03 (m, 4H), 2.92 (br s, 2H), 2.07 (br s, 1H), 2.00-1.84 (m, 4H), 1.70 (br d, J=8.4 Hz, 3H), 1.48-1.34 (m, 3H).

### Step 4: Synthesis of Intermediate 1-8

A mixture of Intermediate 1-6 (0.2 g, 311.93 μmol, 1 eq, HCl salt), Intermediate 1-7 (88.29 mg, 343.12 μmol, 1.1 eq) and HOAt (42.46 mg, 311.93 μmol, 1 eq) in DMF (2 mL) was added EDCI (119.59 mg, 623.86 μmol, 2 eq) and NMM (157.76 mg, 1.56 mmol, 171.48 μL, 5 eq). The mixture was stirred at 25° C. for 1 hr. The mixture was poured into water (6 mL) and filtered. The filter cake was dried under vacuum. Intermediate 1-8 (240 mg, 284.35 μmol, 91.16% yield) was obtained as an off-white solid. LCMS (Method G): Rt=0.684 min, [M+H]+=844.7.

### Step 5: Synthesis of Intermediate 1-9

A mixture of Intermediate 1-8 (240 mg, 284.35 μmol, 1 eq) in DCM (1 mL) was added HCl/dioxane (4 M, 2 mL, 28.13 eq) and the mixture was stirred at 25° C. for 0.5 hr. The mixture was concentrated under vacuum. Intermediate 1-9 (230 mg, crude, HCl salt) was obtained as a brown solid. LCMS (Method G): Rt=0.566 min, [M+H]+=744.5.

### Step 6: Synthesis of Intermediate 1-11

A mixture of Intermediate 1-9 (100 mg, 128.14 μmol, 1 eq, HCl salt), Intermediate 1-10 (73.57 mg, 153.77 μmol, 1.2 eq) and HOAt (17.44 mg, 128.14 μmol, 1 eq) in DMF (1 mL) was added EDCI (49.13 mg, 256.29 μmol, 2 eq) and NMM (64.81 mg, 640.72 μmol, 70.44 μL, 5 eq). The mixture was stirred at 25° C. for 1 hrs. The mixture was poured into water (6 mL) and filtered. The filter cake was dried under vacuum. The residue was purified by Prep-HPLC (column: Waters Xbridge Prep OBD C18 150*40 mm*10 um; mobile phase: [water(NH₄HCO₃)-ACN]; gradient: 45%-75% B over 15 min) and concentrated under vacuum. Intermediate 1-11 (65 mg, 53.97 μmol, 42.12% yield) was obtained as a white solid. LCMS (Method): Rt=2.183 min, [M+H]+=1205.0.

### Step 7: Synthesis of I-640

To a solution of Intermediate 1-11 (64 mg, 53.14 μmol, 1 eq) in DCM (1 mL) was added TFA (153.50 mg, 1.35 mmol, 0.1 mL, 25.33 eq). The mixture was stirred at 25° C. for 2 hr. The mixture was adjusted to pH=8 with NH₃·H₂O and concentrated under vacuum. The residue was purified by Prep-HPLC (column: Waters Xbridge 150*25 mm*5 um; mobile phase: [water (NH₄HCO₃)-ACN]; gradient: 42%-72% B over 9 min) and dried by lyophilization. I-640 (35.65 mg, 28.86 μmol, 54.32% yield, 98.638% purity, TFA salt) was obtained as a white solid. LCMS (Method G): Rt=0.676 min, [M+H]+=1104.5. SFC: Rt=1.022 min. ¹H NMR (400 MHz, CHLOROFORM-d) δ=10.40 (br s, 1H), 8.49-8.39 (m, 1H), 7.92-7.91 (m, 1H), 7.83-7.68 (m, 3H), 7.51-7.40 (m, 3H), 7.37-7.30 (m, 2H), 7.29-7.26 (m, 1H), 7.10-7.01 (m, 1H), 6.92-6.78 (m, 2H), 5.10 (br s, 1H), 4.60-4.51 (m, 1H), 4.49-4.41 (m, 1H), 4.29 (br s, 2H), 4.25-4.14 (m, 3H), 4.08-3.91 (m, 4H), 3.83-3.82 (m, 3H), 3.72-3.63 (m, 2H), 3.62-3.46 (m, 4H), 3.45-3.30 (m, 6H), 3.29-3.12 (m, 2H), 3.10-2.70 (m, 4H), 2.02 (br d, J=4.4 Hz, 2H), 1.93-1.79 (m, 3H), 1.76 (br s, 2H), 1.73-1.53 (m, 5H), 1.34 (br d, J=7.2 Hz, 3H), 1.30-1.01 (m, 5H). ¹⁹F NMR (377 MHz, CHLOROFORM-d) δ=−75.48 (br s, 3F), −109.46-−110.11 (m, 1F), −113.65-−114.11 (m, 1F), −117.44-−118.07 (m, 1F), −118.30 (br d, J=11.2 Hz, 1F).

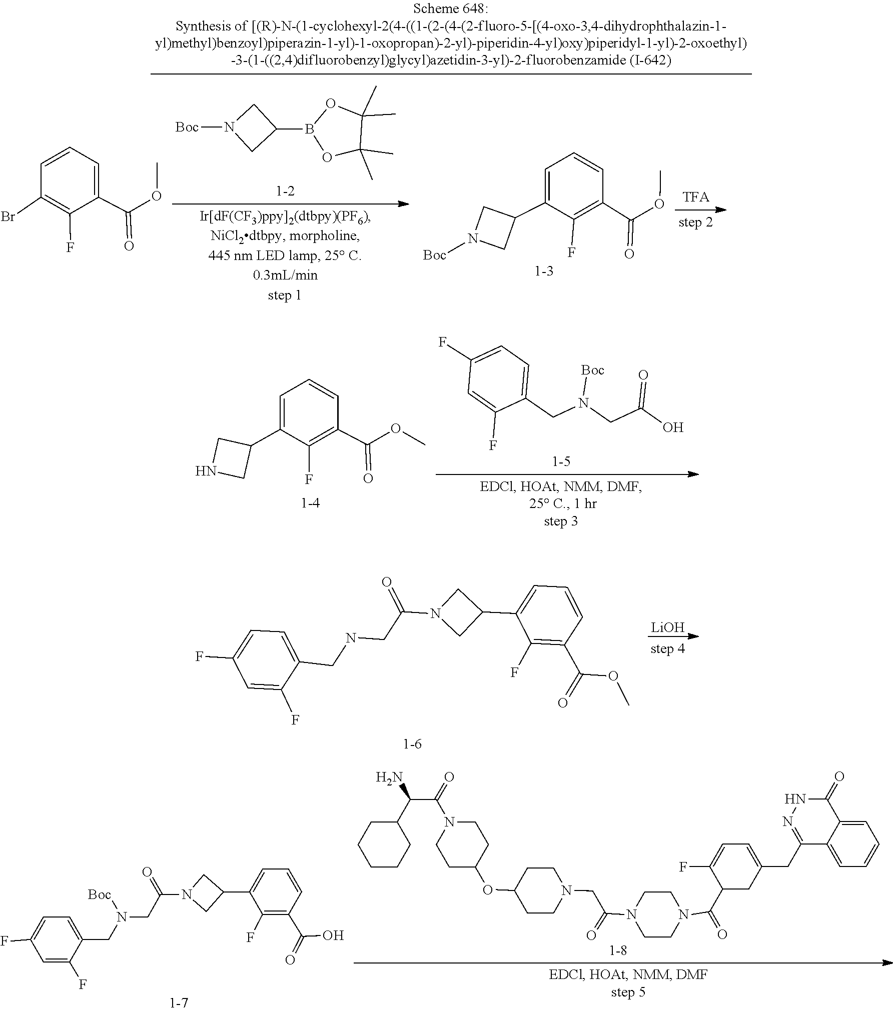

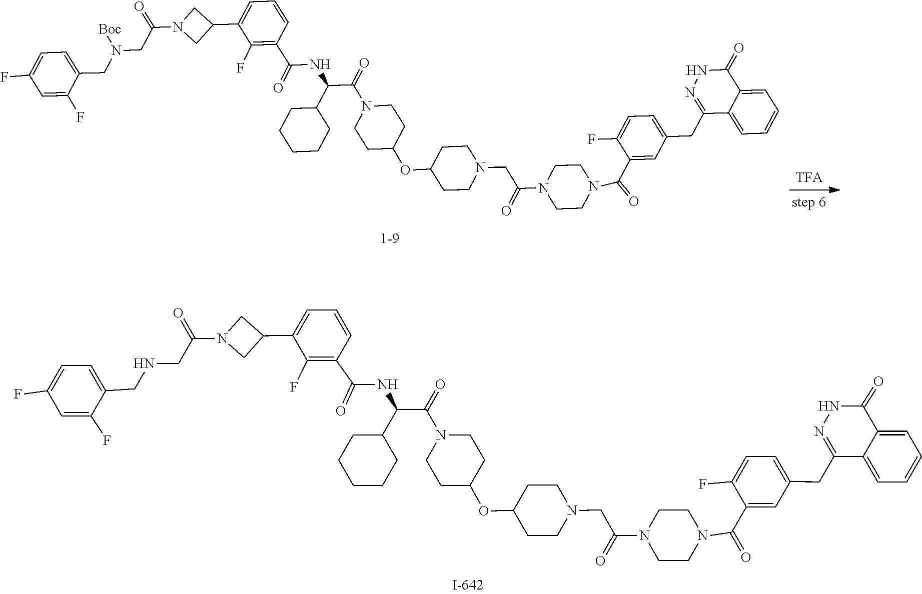 [SMILES: CC(C)(C)OC(=O)N(CC(=O)N1CC(c2cccc(C(=O)N[C@@H](C(=O)N3CCC(OC4CCN(CC(=O)N5CCN(C(=O)c6cc(Cc7n[nH]c(=O)c8ccccc78)ccc6F)CC5)CC4)CC3)C3CCCCC3)c2F)C1)Cc1ccc(F)cc1F ; O=C(N[C@@H](C(=O)N1CCC(OC2CCN(CC(=O)N3CCN(C(=O)c4cc(Cc5n[nH]c(=O)c6ccccc56)ccc4F)CC3)CC2)CC1)C1CCCCC1)c1cccc(C2CN(C(=O)CNCc3ccc(F)cc3F)C2)c1F]

### Step 1: Synthesis of Intermediate 1-3

To an 15 mL vial equipped with a stir bar was added Intermediate 1-1 (0.1 g, 429.12 μmol, 1 eq), Intermediate 1-2 (243.03 mg, 858.24 μmol, 2 eq), bis[3,5-difluoro-2-[5-(trifluoromethyl)-2-pyridyl]phenyl]iridium (1+); 4-tert-butyl-2-(4-tert-butyl-2-pyridyl)pyridine; hexafluorophosphate (4.81 mg, 4.29 μmol, 0.01 eq), 4-tert-butyl-2-(4-tert-butyl-2-pyridyl) pyridine; dichloronickel (8.54 mg, 21.46 μmol, 0.05 eq), and morpholine (56.08 mg, 643.68 μmol, 56.64 μL, 1.5 eq) in DMF (1 mL). The reaction mixture was pumped through a flow reactor at a flow rate of 300 μL min⁻¹, irradiating with a 455 nm LED lamp and water cooling to keep the reaction temperature at 25° C. for 0.5 hr. The reaction mixture was concentrated under reduced pressure to give a residue. The residue was purified by reversed phase chromatography (neutral condition) and the eluent was concentrated to remove ACN and lyophilized to give Intermediate 1-3 (0.16 g, crude) as a yellow solid. LCMS (Method D): Rt=0.394 min, [M+H]⁺=254.1. ¹H NMR (400 MHz, METHANOL-d4) δ=7.86-7.78 (m, 1H), 7.69-7.61 (m, 1H), 7.31-7.27 (m, 1H), 4.37-4.33 (m, 2H), 3.91 (s, 4H), 3.58 (d, J=4.8 Hz, 2H), 2.90-2.76 (m, 1H), 1.46 (s, 9H). ¹⁹F NMR (376 MHz, METHANOL-d₄) δ=−116.91 (s, 1F)

### Step 2: Synthesis of Intermediate 1-4

To a solution of Intermediate 1-3 (0.13 g, 420.26 μmol, 1 eq) in DCM (1.5 mL) was added TFA (143.76 mg, 1.26 mmol, 93.65 μL, 3 eq). The mixture was stirred at 25° C. for 0.5 hr. The reaction mixture was concentrated under reduced pressure to give Intermediate 1-4 (0.13 g, 402.18 μmol, 95.70% yield, TFA salt) as a white solid. LCMS (Method D): Rt=0.227 min, [M+H]⁺=210.9.

### Step 3: Synthesis of Intermediate 1-6

To a solution of Intermediate 1-4 (0.13 g, 402.18 μmol, 1 eq, TFA salt) and Intermediate 1-5 (121.17 mg, 402.18 μmol, 1 eq) in DMF (1.5 mL) was added EDCI (231.29 mg, 1.21 mmol, 3 eq), HOAt (54.74 mg, 402.18 μmol, 1 eq), and NMM (203.40 mg, 2.01 mmol, 221.08 μL, 5 eq). The mixture was stirred at 25° C. for 0.5 hr. The reaction mixture was filtered and concentrated under reduced pressure to give a residue. The residue was purified by prep-HPLC (0.1% FA) the eluent was concentrated to remove ACN and lyophilized to give Intermediate 1-6 (0.1 g, 169.34 μmol, 42.10% yield) as a yellow solid. LCMS (Method D): Rt=0.562 min, [M+H]⁺=494.2.

### Step 4: Synthesis of Intermediate 1-7

To a solution of Intermediate 1-6 (80 mg, 162.44 μmol, 1 eq) in MeOH (0.5 mL), H₂O (0.5 mL), and THF (0.5 mL) was added LiOH·H₂O (20.45 mg, 487.32 μmol, 3 eq). The mixture was stirred at 25° C. for 0.5 hr. The reaction mixture was adjusted to pH=6 by HCl (1M), and then extracted with DCM (3 mL*2). The combined organic layers were washed with brine (2 mL), dried over Na₂SO₄, filtered and concentrated under reduced pressure to give Intermediate 1-7 (80 mg, crude) as a yellow solid. LCMS (Method D): Rt=0.392 min, [M+H]⁺=479.2.

### Step 5: Synthesis of Intermediate 1-9

To a solution of Intermediate 1-7 (80 mg, 167.20 μmol, 1 eq) and Intermediate 1-8 (128.13 mg, 167.20 μmol, 1 eq, HCl salt) in DMF (2 mL) was added EDCI (96.16 mg, 501.61 μmol, 3 eq), HOAt (22.76 mg, 167.20 μmol, 1 eq), and NMM (84.56 mg, 836.02 μmol, 91.91 μL, 5 eq). The mixture was stirred at 25° C. for 0.5 hr. The reaction mixture was filtered and concentrated under reduced pressure to give a residue. The residue was purified by reversed phase chromatography (0.1% TFA). The eluent was concentrated to remove ACN and lyophilized to give Intermediate 1-9 (0.11 g, 92.41 μmol, 55.27% yield) as a white solid. LCMS (Method D): Rt=0.476 min, [M+H]⁺=1190.3. SFC: Rt=1.719 min.

### Step 6: Synthesis of I-642

To a solution of Intermediate 1-9 (0.1 g, 84.01 μmol, 1 eq) in DCM (1 mL) was added TFA (307.00 mg, 2.69 mmol, 200.00 μL, 32.05 eq). The mixture was stirred at 25° C. for 0.5 hr. The reaction mixture was filtered and concentrated under reduced pressure to give a residue. The residue was purified by reversed phase chromatography (0.10% TFA) and the eluent was concentrated to remove ACN and lyophilized to give I-642 (62.52 mg, 48.91 μmol, 58.22% yield, 94.215% purity, TFA salt) as a white solid. LCMS (Method D): Rt=0.381 min, [M+H]⁺=1090.7. SFC: Rt=1.862 min. ¹H NMR (400 MHz, METHANOL-d4) δ=8.39-8.31 (m, 1H), 7.99-7.92 (m, 1H), 7.91-7.80 (m, 2H), 7.68-7.56 (m, 3H), 7.52 (d, J=5.2 Hz, 1H), 7.41-7.28 (m, 2H), 7.22-7.06 (m, 3H), 4.97 (d, J=7.2 Hz, 1H), 4.71-4.60 (m, 1H), 4.54-4.50 (m, 1H), 4.39 (s, 3H), 4.34-4.27 (m, 4H), 4.27-4.20 (m, 2H), 4.06-3.98 (m, 1H), 3.97-3.90 (m, 3H), 3.81 (s, 4H), 3.73 (s, 1H), 3.65 (s, 1H), 3.59-3.42 (m, 5H), 3.36 (s, 3H), 3.22-3.02 (m, 1H), 2.35-2.13 (m, 1H), 2.12-1.93 (m, 4H), 1.91-1.74 (m, 6H), 1.70 (d, J=10.4 Hz, 3H), 1.61-1.48 (m, 1H), 1.34-1.21 (m, 3H), 1.19-1.10 (m, 2H). ¹⁹F NMR (377 MHz, METHANOL-d4) δ=−77.20 (s, 7F), −108.24 (s, 1F), −112.92 (d, J=8.4 Hz, 1F), −120.42-−120.63 (m, 1F), −120.71 (d, J=31.2 Hz, 1F).

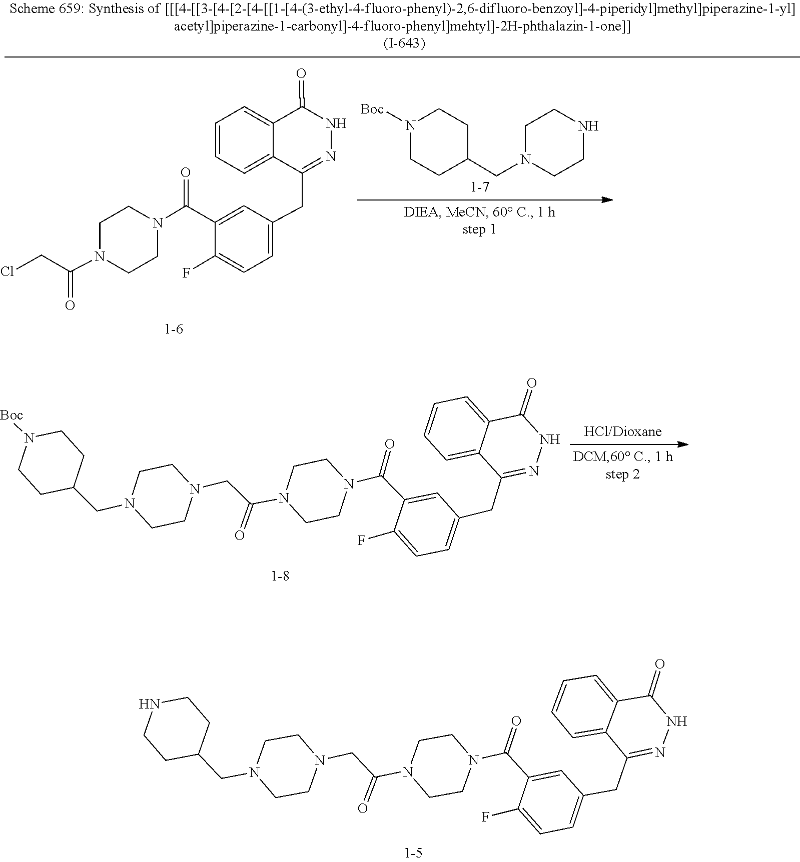 [SMILES: O=C(CCl)N1CCN(C(=O)c2cc(Cc3n[nH]c(=O)c4ccccc34)ccc2F)CC1 ; CC(C)(C)OC(=O)N1CCC(CN2CCNCC2)CC1 ; CC(C)(C)OC(=O)N1CCC(CN2CCN(CC(=O)N3CCN(C(=O)c4cc(Cc5n[nH]c(=O)c6ccccc56)ccc4F)CC3)CC2)CC1 ; O=C(CN1CCN(CC2CCNCC2)CC1)N1CCN(C(=O)c2cc(Cc3n[nH]c(=O)c4ccccc34)ccc2F)CC1]

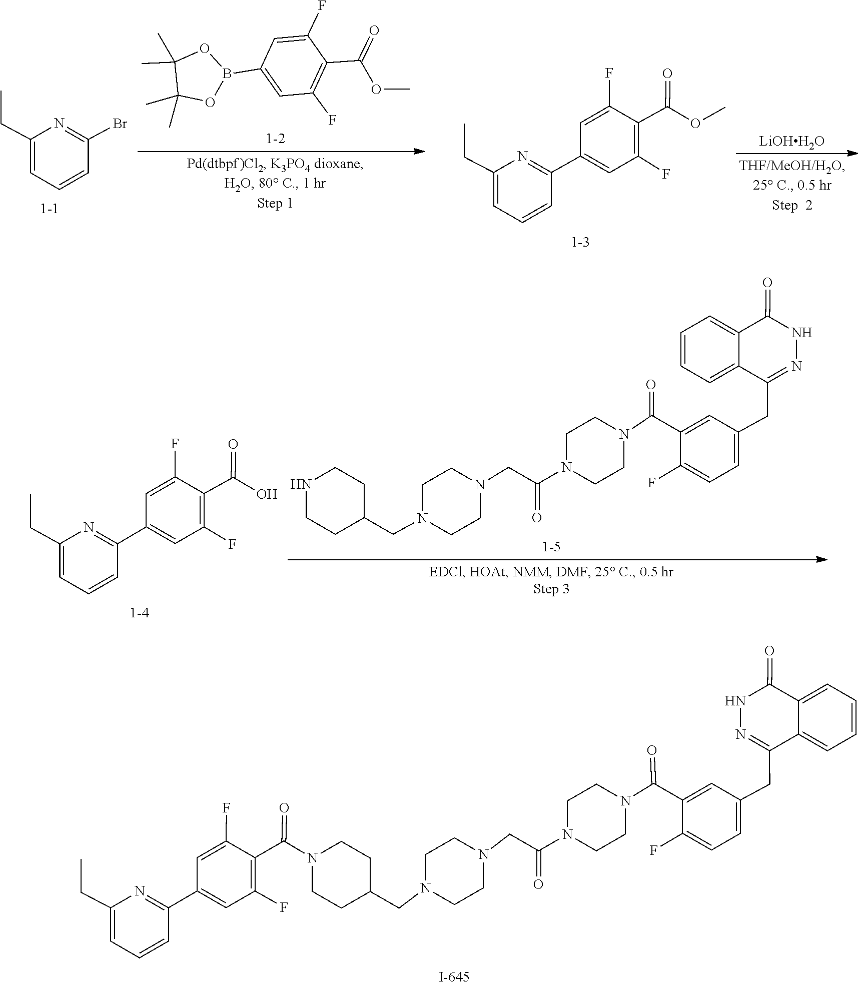

### Step 1: Synthesis of Intermediate 1-8.

To a solution of Intermediate 1-7 (767.92 mg, 2.71 mmol, 1.2 eq) in ACN (10 mL) was added DIEA (1.17 g, 9.03 mmol, 1.57 mL, 4 eq) and Intermediate 1-6 (1 g, 2.26 mmol, 1 eq). The mixture was stirred at 60° C. for 1 hr. The mixture was concentrated under reduced pressure to give a residue. The residue was purified by flash silica gel chromatography (ISCO; 20 g SepaFlash Silica Flash Column, Eluent of 0˜100% DCM/MeOH@50 mL/min, DCM/MeOH=10:1, Rf=0.4) and the eluent was concentrated under reduced pressure to give Intermediate 1-8 (1.2 g, 1.74 mmol, 77.04% yield) as a yellow solid. LCMS (Method D): Rt=0.327 min, [M+H]⁺=690.3.

### Step 2: Synthesis of Intermediate 1-5.

To a solution oft Intermediate 1-8 (1 g, 1.45 mmol, 1 eq) in DCM (1 mL) was added HCl/dioxane (2 M, 10.00 mL, 13.80 eq). The mixture was stirred at 25° C. for 1 hr. The mixture was concentrated under reduced pressure to give Intermediate 1-5 (1 g, crude, HCl salt) as a yellow solid. LCMS (Method D): Rt=0.214 min, [M+H]⁺=590.2.

### Step 3: Synthesis of Intermediate 1-3.

A mixture of Intermediate 1-1 (100 mg, 492.49 μmol, 1 eq), Intermediate 1-2 (161.49 mg, 541.74 μmol, 1.1 eq), Pd(dtbpf)Cl₂ (32.10 mg, 49.25 μmol, 0.1 eq), and K₃PO₄ (313.61 mg, 1.48 mmol, 3 eq) in dioxane (1 mL) and H₂O (0.2 mL) was degassed and purged with N₂ 3 times, and then the mixture was stirred at 80° C. for 1 hr under N₂ atmosphere. The reaction mixture was poured into water (5 mL) and extracted with EA (3 mL*3). The combined organic layers were dried over anhydrous Na₂SO₄, filtered and concentrated under reduced pressure to give a residue. The residue was purified by flash silica gel chromatography (ISCO; 12 g SepaFlash Silica Flash Column, Eluent of 0˜100% PE/EA@30 mL/min, PE/EA=10:1, Rf=0.8) and the eluent was concentrated under reduced pressure to give Intermediate 1-3 (140 mg, 475.76 μmol, 96.60% yield, 100% purity) as yellow oil. LCMS (Method D): Rt=0.516 min, [M+H]⁺=295.1. ¹H NMR (400 MHz, CHLOROFORM-d) δ=7.31-7.27 (m, 1H), 7.26-7.22 (m, 1H), 7.22-7.13 (m, 3H), 3.98 (s, 3H), 2.78-2.71 (m, 2H), 1.30-1.27 (m, 3H). ¹⁹FNMR (400 MHz, CHLOROFORM-d) δ=−110.308, −123.540.

### Step 4: Synthesis of Intermediate 1-4.

To a solution of Intermediate 1-3 (130 mg, 441.77 μmol, 1 eq) in THF (0.5 mL), MeOH (0.5 mL) and H₂O (0.5 mL) was added LiOH·H₂O (55.62 mg, 1.33 mmol, 3 eq). The mixture was stirred at 25° C. for 0.5 hr. The reaction pH was adjusted to pH=5 with 1M HCl, and then diluted with H₂O (20 mL), extracted with EA (15 mL*5), dried by Na₂SO₄, filtered and concentrated to give Intermediate 1-4 (120 mg, crude) as a white solid. LCMS (Method D): Rt=0.513 min, [M+H]⁺=281.0.

### Step 5: Synthesis of I-643.

To a solution of Intermediate 1-4 (50 mg, 178.42 μmol, 1 eq) in DMF (1 mL) was added EDCI (102.61 mg, 535.25 μmol, 3 eq), HOAt (24.28 mg, 178.42 μmol, 1 eq), NMM (90.23 mg, 892.09 μmol, 98.08 μL, 5 eq) and Intermediate 1-5 (111.72 mg, 178.42 μmol, 1 eq, HCl salt). The mixture was stirred at 25° C. for 0.5 hr. The reaction mixture was poured into water (5 mL) and extracted with EA (3 mL*3). The combined organic layers were dried over Na₂SO₄, filtered and concentrated under reduced pressure to give a residue. The residue was purified by prep-HPLC (column: CD01-Phenomenex luna C18 150*25*10 um; mobile phase: [water (FA)-ACN]; gradient: 22%-52% B over 11 min) and the eluent was concentrated under reduced pressure to remove ACN, and lyophilized to give I-643 (55.73 mg, 60.52 mol, 33.92% yield, 97.519% purity, FA salt) as a white solid. LCMS (Method D): Rt=0.400 min, [M+H]⁺=852.2. ¹H NMR (400 MHz, METHANOL-d4) δ=8.44 (s, 1H), 8.39-8.34 (m, 1H), 7.99-7.92 (m, 1H), 7.91-7.79 (m, 2H), 7.49 (d, J=2.8 Hz, 1H), 7.40-7.33 (m, 3H), 7.32-7.27 (m, 2H), 7.23-7.19 (m, 1H), 7.16 (t, J=8.8 Hz, 1H), 4.70 (d, J=12.8 Hz, 1H), 4.38 (s, 2H), 3.82-3.72 (m, 2H), 3.71-3.58 (m, 3H), 3.52 (s, 2H), 3.42 (s, 1H), 3.34 (s, 2H), 3.25-3.17 (m, 1H), 2.93 (t, J=12.8 Hz, 1H), 2.87-2.73 (m, 4H), 2.69 (d, J=19.6 Hz, 7H), 2.46-2.40 (m, 2H), 2.02-1.92 (m, 2H), 1.83 (d, J=12.8 Hz, 1H), 1.26 (t, J=7.6 Hz, 3H), 1.23-1.08 (m, 2H). ¹⁹F NMR (400 MHz, METHANOL-d4) δ=−115.614, −120.777, −126.263.

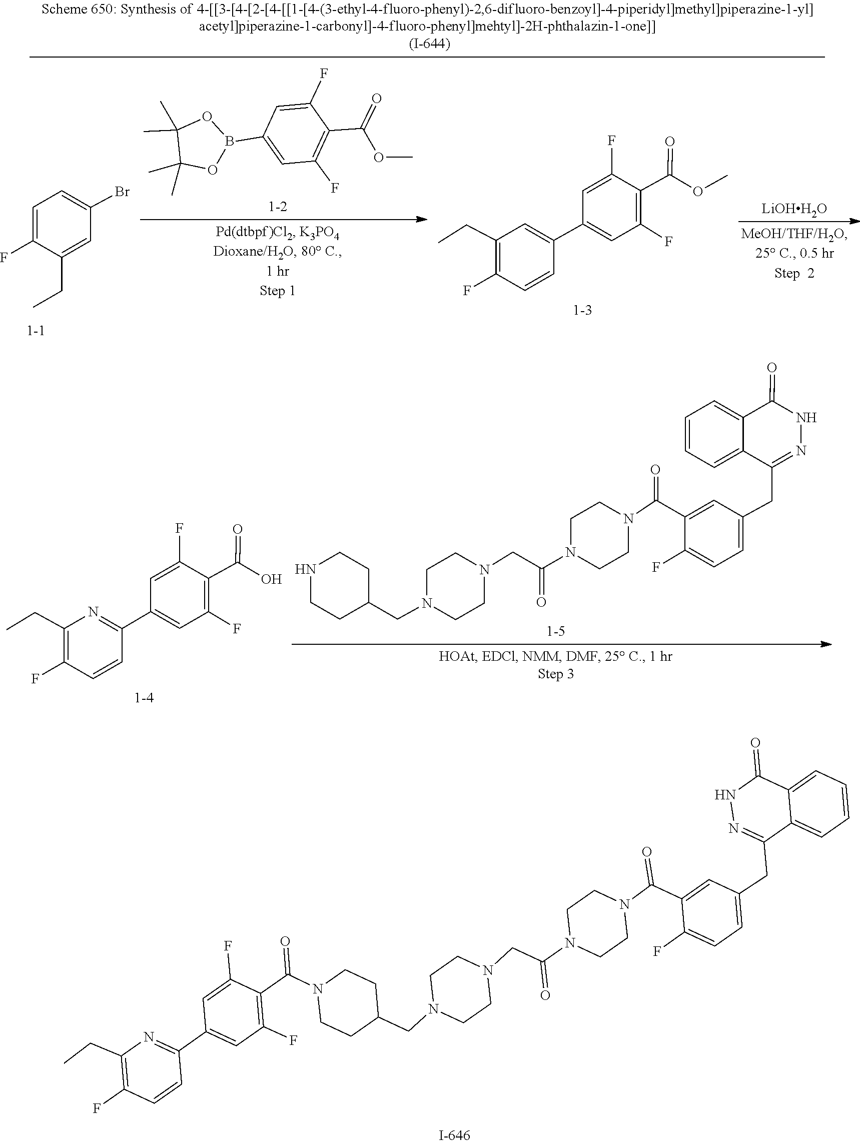

### Step 1: Synthesis of Intermediate 1-3

A mixture of Intermediate 1-1 (100 mg, 492.49 μmol, 1 eq), Intermediate 1-2 (161.49 mg, 541.74 μmol, 1.1 eq), K₃PO₄ (313.61 mg, 1.48 mmol, 3 eq) and Pd(dtbpf)Cl₂ (32.10 mg, 49.25 μmol, 0.1 eq) in dioxane (1 mL) and H₂O (0.2 mL) was degassed and purged with N₂ 3 times, and then the mixture was stirred at 80° C. for 1 hr under N₂ atmosphere. The residue was diluted with H₂O (5 mL) and extracted with EA (5 mL*3), dried over Na₂SO₄, filtered and concentrated under reduced pressure to give a residue. The residue was purified by flash silica gel chromatography (ISCO; 12 g SepaFlash Silica Flash Column, Eluent of 0˜100% Petroleum ether/Ethyl acetate @30 mL/min). The eluent was concentrated under reduced pressure to give Intermediate 1-3 (140 mg, 475.76 μmol, 96.60% yield) as a yellow solid. LCMS (Method): Rt=0.506 min, [M+H]⁺=294.9.

### Step 2: Synthesis of Intermediate 1-4

To a solution of Intermediate 1-3 (140 mg, 475.76 μmol, 1 eq) in MeOH (0.4 mL), THF (0.4 mL), and H₂O (0.4 mL) was added LiOH·H₂O (59.89 mg, 1.43 mmol, 3 eq). The mixture was stirred at 25° C. for 1 hr. The mixture was adjusted to pH=5 with HCl (2M) and extracted with EA (5 mL*3), dried over Na₂SO₄, filtered and concentrated under reduced pressure to give a residue. The crude product was used in the next step without further purification. Intermediate 1-4 (120 mg, 428.20 μmol, 90.00% yield) was obtained as a white solid. LCMS (Method D): Rt=0.459 min, [M+H]⁺=281.0.

### Step 3: Synthesis of I-644

To a solution of Intermediate 1-4 (30 mg, 107.05 μmol, 1 eq) in DMF (0.5 mL) was added HOAt (14.57 mg, 107.05 μmol, 1 eq), EDCI (61.57 mg, 321.15 μmol, 3 eq), NMM (54.14 mg, 535.25 μmol, 58.85 L, 5 eq) and Intermediate 1-5 (80.44 mg, 128.46 μmol, 1.2 eq, HCl salt). The mixture was stirred at 25° C. for 1 hr. The reaction liquid was concentrated under reduced pressure to obtain the crude product. The residue was purified by prep-HPLC (column: CD01-Phenomenex luna C18 150*25*10 um; mobile phase: [water (FA)-ACN]; gradient: 20%-500% B over 10 min), which was concentrated and lyophilized to afford I-644 (38.14 mg, 42.47 μmol, 39.68% yield, 100.00% purity, FA salt) as a white solid. LCMS (Method D): Rt=0.410 min, [M+H]⁺=852.3. ¹H NMR (400 MHz, METHANOL-d4) δ=8.42 (s, 1H), 8.36 (d, J=7.6 Hz, 1H), 7.99-7.91 (m, 1H), 7.90-7.78 (m, 2H), 7.58-7.55 (m, 1H), 7.54-7.45 (m, 2H), 7.38-7.35 (m, 3H), 7.21-7.08 (m, 2H), 4.85-4.78 (m, 1H), 4.69 (d, J=12.4 Hz, 1H), 4.38 (s, 2H), 3.85-3.71 (m, 2H), 3.71-3.57 (m, 3H), 3.56-3.48 (m, 2H), 3.46-3.33 (m, 3H), 3.20 (t, J=12.8 Hz, 1H), 2.92 (t, J=12.4 Hz, 1H), 2.82-2.56 (m, 10H), 2.50-2.38 (m, 2H), 2.04-1.89 (m, 2H), 1.82 (d, J=12.8 Hz, 1H), 1.27 (t, J=7.6 Hz, 3H), 1.24-1.06 (m, 2H). ¹⁹F NMR (377 MHz, METHANOL-d₄) δ=−115.231, −120.770, −121.233.

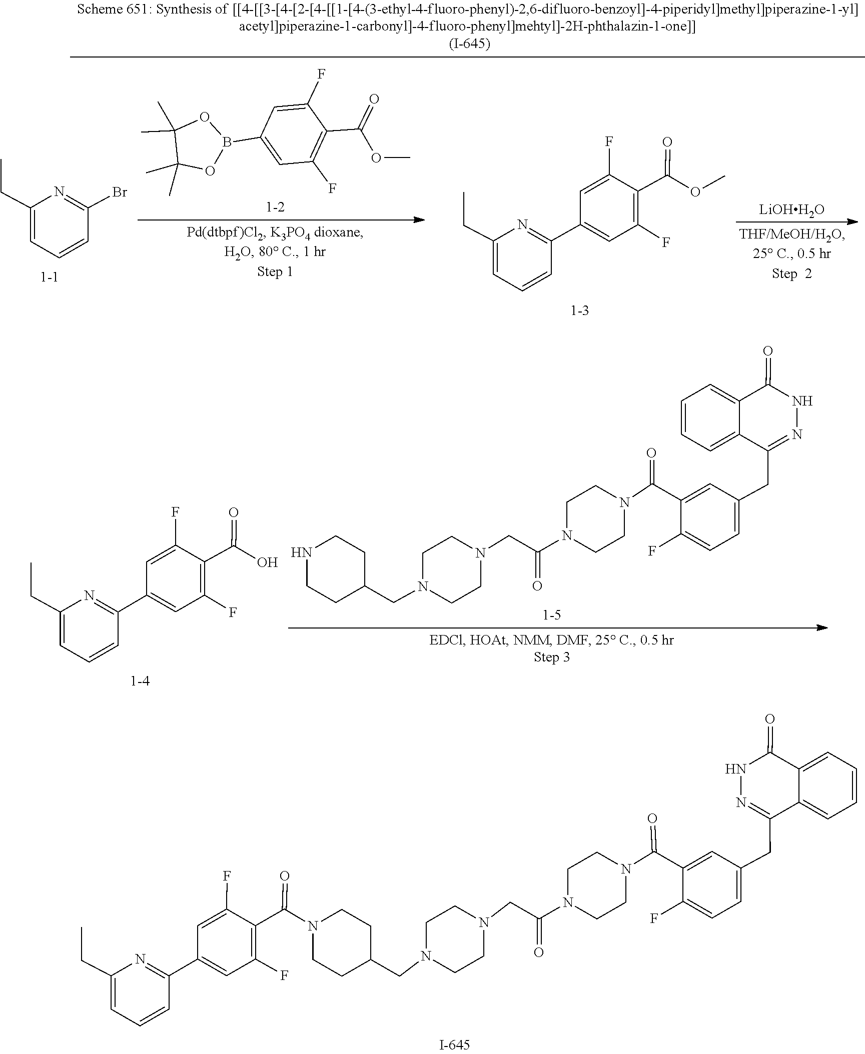

### Step 1: Synthesis of Intermediate 1-3.

To a solution of Intermediate 1-1 (200 mg, 1.07 mmol, 1 eq) and Intermediate 1-2 (384.53 mg, 1.29 mmol, 1.2 eq) in dioxane (2 mL) and H₂O (0.4 mL) was added ditert-butyl(cyclopenta-1,4-dien-1-yl) phosphane; dichloropalladium; iron (70.06 mg, 107.50 μmol, 0.1 eq) and K₃PO₄ (684.55 mg, 3.22 mmol, 3 eq). The mixture was stirred at 80° C. for 1 hr. The mixture was diluted with water (5 mL) and extracted with DCM (5 mL*3). The combined organic layer was dried over Na₂SO₄, filtered and concentrated in vacuo to give a residue. The residue was purified by column chromatography (SiO₂, PE:EA=20:1 to PE:EA=0:1), then the organic liquid was concentrated in vacuo to give Intermediate 1-3 (250 mg, 901.66 μmol, 83.88% yield) as a yellow solid. LCMS (Method D): Rt=0.262 min, [M+H]⁺=278.2.

### Step 2: Synthesis of Intermediate 1-4.

To a solution of Intermediate 1-3 (200 mg, 721.33 μmol, 1 eq) in MeOH (0.4 mL), THF (0.4 mL), and H₂O (0.4 mL) was added LiOH·H₂O (90.81 mg, 2.16 mmol, 3 eq). The mixture was stirred at 25° C. for 0.5 hr. The reaction mixture was concentrated in vacuo to remove THF and MeOH to give a residue, the residue was diluted with water (5 mL) and extracted with DCM (5 mL*3), then the aqueous phase was acidified with hydrochloric acid (1 M) to pH=5-6, and then the mixture was extracted with DCM (5 mL*3). The combined organic layer was dried over Na₂SO₄, filtered and concentrated in vacuo to give a residue. The residue was used in the next step directly without further purification. Intermediate 1-4 (150 mg, 569.82 μmol, 79.00% yield) was obtained as a white solid. LCMS (Method D): Rt=0.144 min, [M+H]⁺=264.1.

### Step 3: Synthesis of I-645.

To a solution of Intermediate 1-4 (50 mg, 189.94 μmol, 1 eq) and Intermediate 1-5 (130.83 mg, 208.94 μmol, 1.1 eq, HCl salt) in DMF (1 mL) was added EDCI (109.24 mg, 569.82 μmol, 3 eq), HOAt (25.85 mg, 189.94 μmol, 26.57 μL, 1 eq), and NMM (96.06 mg, 949.71 μmol, 104.41 μL, 5 eq). The mixture was stirred at 25° C. for 1 hr. The mixture was diluted with water (5 mL) and extracted with DCM (5 mL*3). The combined organic layer was dried over Na₂SO₄, filtered and concentrated in vacuo to give a residue. The residue was purified by prep-HPLC (column: CD01-Phenomenex luna C18 150*25*10 um; mobile phase: [water (FA)-ACN]; gradient: 2%-32% B over 10 min). Then the eluent was concentrated in vacuo and lyophilized. I-645 (60.71 mg, 68.48 μmol, 36.05% yield, 99.374% purity, FA salt) was obtained as a white solid. LCMS (Method D): Rt=0.284 min, [M+H]⁺=835.7. ¹H NMR (400 MHz, METHANOL-d4) δ=8.53 (d, J=5.6 Hz, 1H), 8.36 (d, J=7.2 Hz, 1H), 8.32 (s, 1H), 7.94 (d, J=7.6 Hz, 1H), 7.89-7.80 (m, 2H), 7.65 (s, 1H), 7.59-7.54 (m, 3H), 7.50 (s, 1H), 7.37-7.34 (m, 1H), 7.17 (t, J=9.2 Hz, 1H), 4.71 (d, J=13.2 Hz, 1H), 4.38 (s, 2H), 3.80-3.73 (m, 2H), 3.69-3.60 (m, 3H), 3.56 (s, 1H), 3.53-3.47 (m, 3H), 3.34 (s, 1H), 3.23 (t, J=12.8 Hz, 2H), 2.99-2.91 (m, 3H), 2.90 (d, J=7.6 Hz, 3H), 2.87 (s, 5H), 2.65-2.59 (m, 2H), 2.04 (d, J=2.4 Hz, 1H), 1.96 (d, J=13.6 Hz, 1H), 1.83 (d, J=12.4 Hz, 1H), 1.34 (t, J=7.6 Hz, 3H), 1.27-1.23 (m, 2H). ¹⁹F NMR (376 MHz, METHANOL-d₄) δ=−114.347, −120.667.

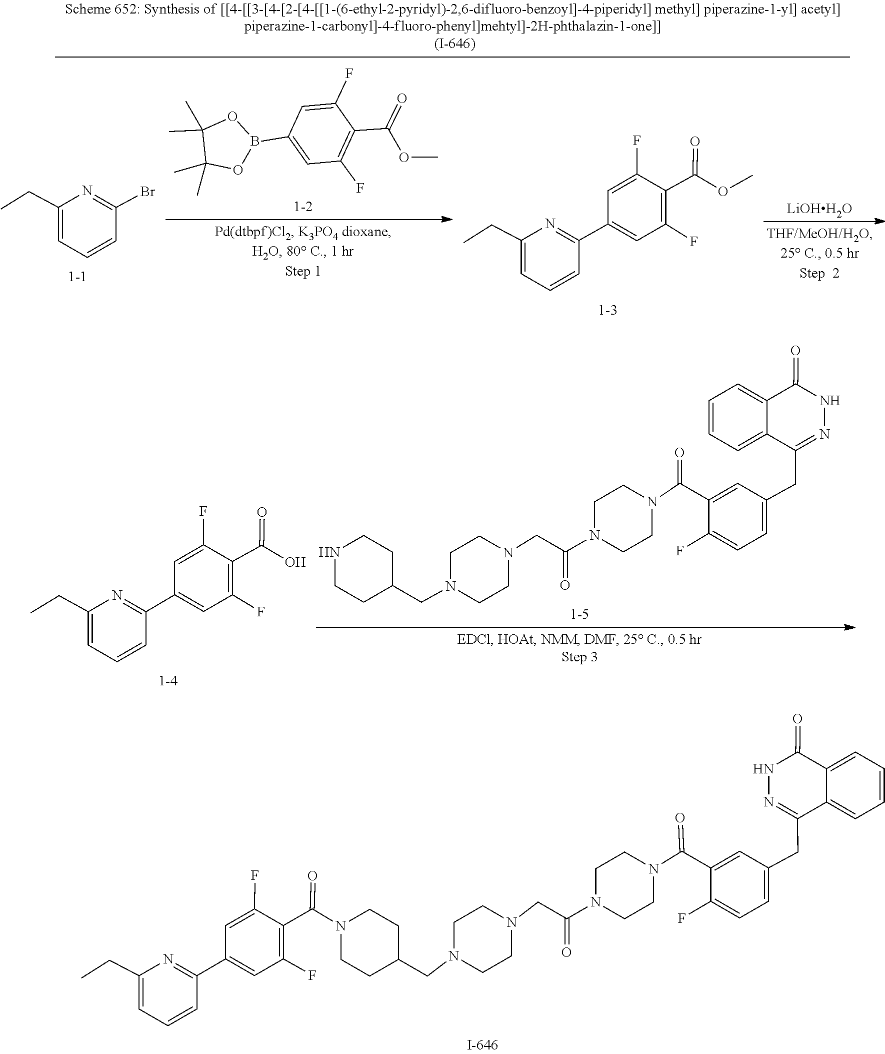

### Step 1: Synthesis of Intermediate 1-3

A mixture of intermediate 1-1 (100 mg, 537.49 μmol, 1 eq), intermediate 1-2 (160.22 mg, 537.49 μmol, 1 eq), Pd(dtbpf)Cl₂ (35.03 mg, 53.75 μmol, 0.1 eq), and K₃PO₄ (342.28 mg, 1.61 mmol, 3 eq) in dioxane (1 mL) and H₂O (0.2 mL) was degassed and purged with N₂ 3 times, and then the mixture was stirred at 80° C. for 1 hr under N₂ atmosphere. The reaction mixture was concentrated under reduced pressure to give a residue. The residue was purified by flash silica gel chromatography (ISCO; 12 g SepaFlashSilica Flash Column, Eluent of 0˜100% PE/EA@36 mL/min, PE/EA=10:1, Rf=0.5) and the eluent was concentrated under reduced pressure to give a product. Intermediate 1-3 (140 mg, crude) was obtained as yellow gum. LCMS (Method D): Rt=0.363 min, [M+H]⁺=278.0.

### Step 2: Synthesis of Intermediate 1-4

To a solution of intermediate 1-3 (140 mg, 504.93 μmol, 1 eq) in MeOH (1 mL), THF (1 mL) and H₂O (1 mL) was added LiOH H₂O (63.57 mg, 1.51 mmol, 3 eq). The mixture was stirred at 25° C. for 0.5 hr. The reaction mixture was poured into H₂O (2 mL) at 25° C., the pH was adjusted to 7 with 1M aqueous HCl, and then extracted with EA (2 mL*3). The combined organic layers were dried over Na₂SO₄, filtered and concentrated under reduced pressure to give a product. The product was used for the next step without further purification. Intermediate 1-4 (90 mg, 308.53 μmol, 61.10% yield, 90.24% purity) was obtained as a yellow gum. LCMS (Method D): Rt=0.190 min, [M+H]⁺=264.1.

### Step 3: Synthesis of I-646

To a solution of intermediate 1-4 (30 mg, 113.96 μmol, 1 eq) and intermediate 1-5 (71.36 mg, 113.96 μmol, 1 eq, HCl salt) in DMF (0.7 mL) was added EDCI (65.54 mg, 341.89 μmol, 3 eq), HOAt (15.51 mg, 113.96 μmol, 15.94 μL, 1 eq) and NMM (57.64 mg, 569.82 μmol, 62.65 μL, 5 eq). The mixture was stirred at 25° C. for 0.5 hr. The reaction mixture was concentrated under reduced pressure to give a residue. The residue was purified by prep-HPLC (column: CD01-Phenomenex luna C18 150*25*10 um; mobile phase: [water (FA)-ACN]; gradient: 12%-42% B over 10 min) and the eluent was concentrated to remove MeCN and then lyophilized. I-646 (15.96 mg, 17.86 μmol, 15.68% yield, 98.61% purity, FA salt) was obtained as a white solid. LCMS (Method D): Rt=0.342 min, [M+H]⁺=835.2. ¹H NMR (400 MHz, METHANOL-d4) δ=8.45-8.40 (m, 1H), 8.40-8.33 (m, 1H), 7.98-7.91 (m, 1H), 7.90-7.85 (m, 1H), 7.85-7.76 (m, 4H), 7.76-7.72 (m, 1H), 7.49 (s, 1H), 7.41-7.33 (m, 1H), 7.30 (d, J=7.6 Hz, 1H), 7.17 (t, J=8.8 Hz, 1H), 4.70 (d, J=12.0 Hz, 1H), 4.39 (s, 2H), 3.82-3.72 (m, 2H), 3.71-3.60 (m, 3H), 3.55-3.48 (m, 2H), 3.42 (s, 1H), 3.35-3.33 (m, 2H), 3.26-3.11 (m, 2H), 3.00-2.80 (m, 4H), 2.70 (d, J=14.8 Hz, 7H), 2.49-2.39 (m, 2H), 2.02-1.90 (m, 2H), 1.82 (t, J=12.4 Hz, 1H), 1.35 (t, J=7.6 Hz, 3H), 1.27-1.15 (m, 2H). ¹⁹F NMR (377 MHz, METHANOL-d4) δ=−115.270, −120.779.

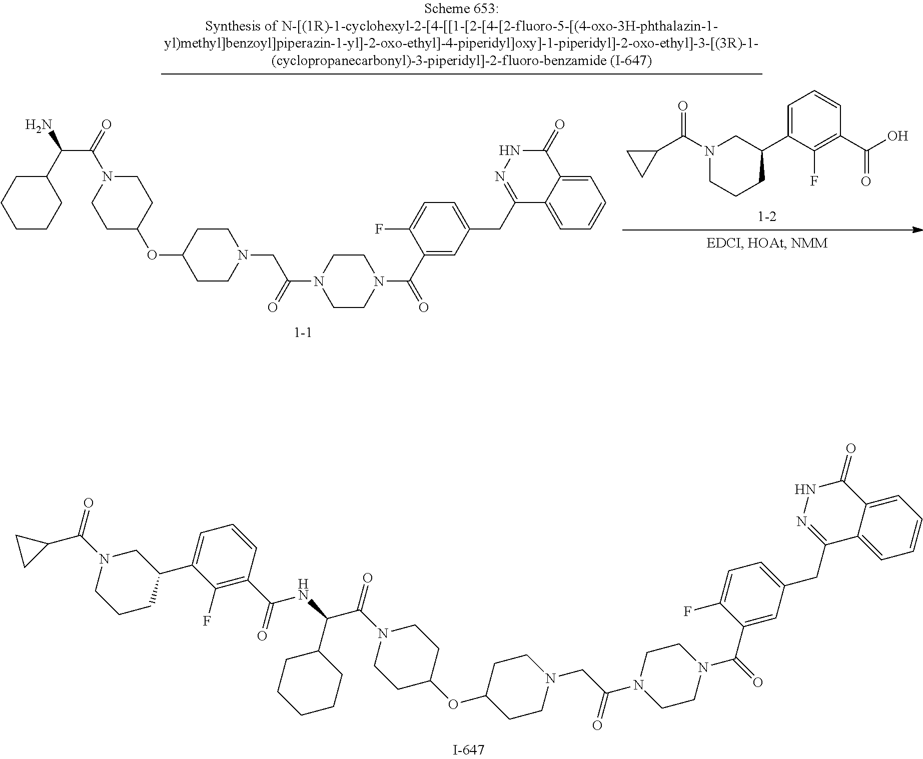 [SMILES: N[C@@H](C(=O)N1CCC(OC2CCN(CC(=O)N3CCN(C(=O)c4cc(Cc5n[nH]c(=O)c6ccccc56)ccc4F)CC3)CC2)CC1)C1CCCCC1 ; O=C(O)c1cccc([C@H]2CCCN(C(=O)C3CC3)C2)c1F ; O=C(N[C@@H](C(=O)N1CCC(OC2CCN(CC(=O)N3CCN(C(=O)c4cc(Cc5n[nH]c(=O)c6ccccc56)ccc4F)CC3)CC2)CC1)C1CCCCC1)c1cccc([C@@H]2CCCN(C(=O)C3CC3)C2)c1F]

To a solution of intermediate 1-1 (50 mg, 65.25 μmol, 1 eq, HCl salt) and intermediate 1-2 (19.01 mg, 65.25 μmol, 1 eq) in DMF (1 mL) was added EDCI (25.02 mg, 130.49 μmol, 2 eq), NMM (33.00 mg, 326.23 μmol, 35.87 μL, 5 eq) and HOAt (8.88 mg, 65.25 μmol, 1 eq). The mixture was stirred at 25° C. for 1 hr. The reaction mixture was extracted with EtOAc (3 mL*3). The combined organic layers were washed with brine (5 mL*2), dried over Na₂SO₄, filtered and concentrated under reduced pressure to give a residue. The residue was purified by prep-HPLC (column: Phenomenex luna C18 150*25 mm*10 um; mobile phase: [water(FA)-ACN]; gradient: 23%-53% B over 10 min). I-647 (35 mg, 33.36 μmol, 51.13% yield, FA salt) was obtained as a white solid. LCMS (Method E): Rt=0.495 min, M+H=1003.8. SFC: Rt=2.486 min, ee %=99.708%. ¹H NMR (400 MHz, DMSO-d6) δ=12.59 (s, 1H), 8.47-8.31 (m, 1H), 8.26 (d, J=8.0 Hz, 1H), 8.13 (s, 1H), 8.00-7.94 (m, 1H), 7.92-7.81 (m, 2H), 7.52-7.35 (m, 4H), 7.26-7.21 (m, 2H), 4.86-4.71 (m, 1H), 4.51-4.25 (m, 4H), 4.05-3.78 (m, 2H), 3.71-3.33 (m, 10H), 3.29-2.95 (m, 8H), 2.93-2.79 (m, 1H), 2.77-2.58 (m, 3H), 2.19-2.11 (m, 1H), 2.03-1.86 (m, 3H), 1.86-1.65 (m, 10H), 1.61-1.55 (m, 2H), 1.46-1.34 (m, 3H), 1.18-1.10 (m, 2H), 1.07-0.95 (m, 2H), 0.79-0.62 (m, 4H). ¹⁹F NMR (377 MHz, DMSO-d6) δ=−119.77 (br s, 1F), −120.11-−120.47 (m, 1F).

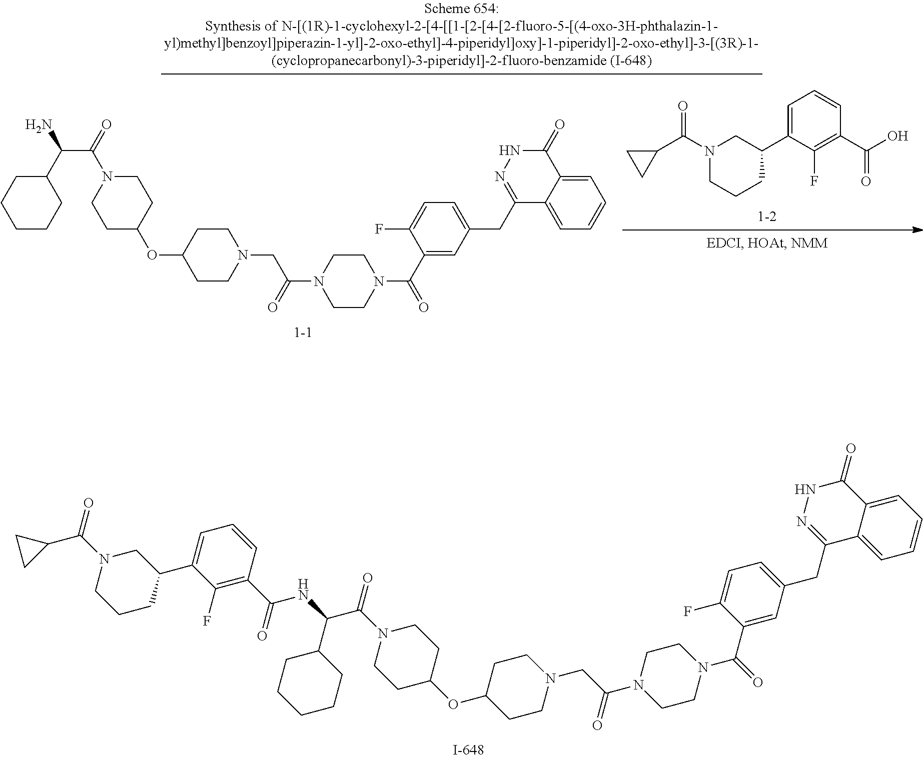 [SMILES: N[C@@H](C(=O)N1CCC(OC2CCN(CC(=O)N3CCN(C(=O)c4cc(Cc5n[nH]c(=O)c6ccccc56)ccc4F)CC3)CC2)CC1)C1CCCCC1 ; O=C(O)c1cccc([C@@H]2CCCN(C(=O)C3CC3)C2)c1F ; O=C(N[C@@H](C(=O)N1CCC(OC2CCN(CC(=O)N3CCN(C(=O)c4cc(Cc5n[nH]c(=O)c6ccccc56)ccc4F)CC3)CC2)CC1)C1CCCCC1)c1cccc([C@@H]2CCCN(C(=O)C3CC3)C2)c1F]

To a solution of intermediate 1-1 (50 mg, 65.25 μmol, 1 eq, HCl salt) and intermediate 1-2 (19.01 mg, 65.25 μmol, 1 eq) in DMF (1 mL) was added EDCI (25.02 mg, 130.49 μmol, 2 eq), NMM (33.00 mg, 326.23 μmol, 35.87 μL, 5 eq) and HOAt (8.88 mg, 65.25 μmol, 1 eq). The mixture was stirred at 25° C. for 1 hr. The reaction mixture was extracted with EtOAc (3 mL*3). The combined organic layers were washed with brine (5 mL*2), dried over Na₂SO₄, filtered and concentrated under reduced pressure to give a residue. The residue was purified by prep-HPLC (column: Phenomenex luna C18 150*25 mm*10 um; mobile phase: [water(FA)-ACN]; gradient: 23%-53% B over 10 min). I-648 (34 mg, 32.41 μmol, 49.67% yield, FA salt) was obtained as a white solid. LCMS (Method E): Rt=0.487 min, M+H=1003.8. SFC: Rt=3.321 min, ee %=99.663%. ¹H NMR (400 MHz, DMSO-d6) δ=12.59 (s, 1H), 8.44-8.32 (m, 1H), 8.29-8.23 (m, 1H), 8.14 (s, 1H), 7.99-7.94 (m, 1H), 7.91-7.80 (m, 2H), 7.51-7.34 (m, 4H), 7.26-7.21 (m, 2H), 4.89-4.66 (m, 1H), 4.49-4.37 (m, 1H), 4.36-4.22 (m, 3H), 4.03-3.80 (m, 2H), 3.73-3.35 (m, 10H), 3.28-2.56 (m, 12H), 2.23-2.14 (m, 1H), 2.01-1.56 (m, 15H), 1.52-1.49 (m, 1H), 1.52-1.38 (m, 2H), 1.14 (br s, 2H), 1.08-0.98 (m, 2H), 0.77-0.63 (m, 4H). ¹⁹F NMR (377 MHz, DMSO-d6) δ=−119.77 (br s, 1F), −120.11-−120.47 (m, 1F).

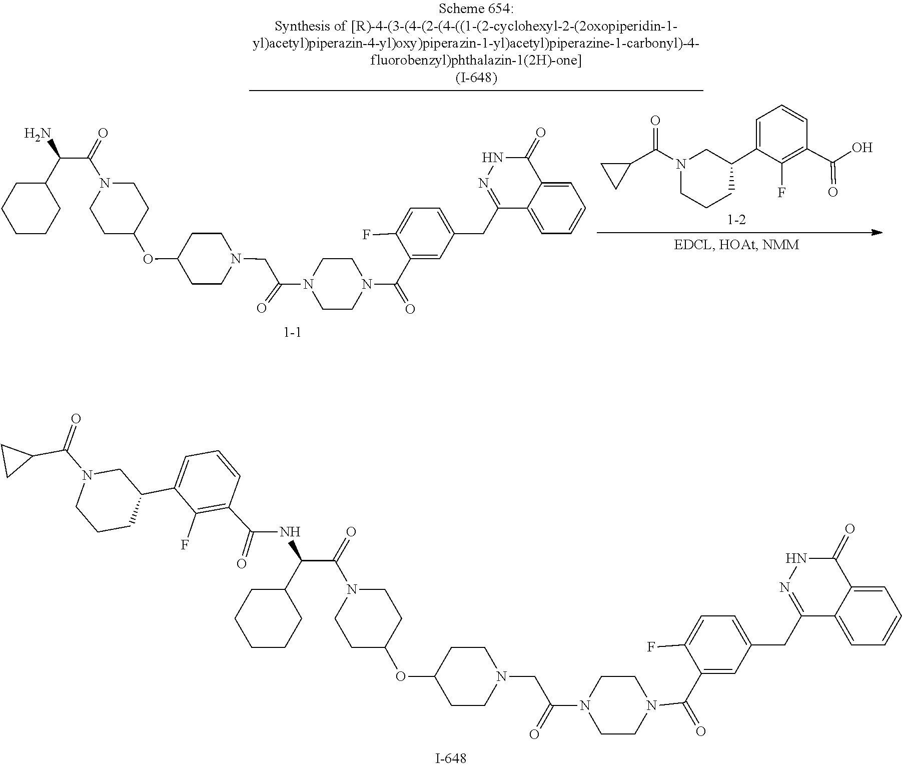

### Step 1: Synthesis of Intermediate 1-3.

To a solution of Intermediate 1-1 (150 mg, 195.74 μmol, 1 eq, HCl salt) and Intermediate 1-2 (25.47 mg, 195.74 μmol, 1 eq) in MeOH (1 mL) was added TEA (118.84 mg, 1.17 mmol, 163.46 μL, 6 eq) and HOAc (70.53 mg, 1.17 mmol, 67.23 μL, 6 eq) and the mixture was stirred at 40° C. for 6 hr. Then NaBH₃CN (49.20 mg, 782.94 μmol, 4 eq) was added, and the mixture was stirred at 40° C. for 6 hr. The reaction mixture was diluted with H₂O (2 mL) and extracted with EA (5 mL*3). The combined organic layers were concentrated under reduced pressure to give a residue. The crude product was purified by reversed-phase chromatography (0.1% FA condition), and the eluent was concentrated and lyophilized to give the desired product. Intermediate 1-3 (90 mg, 99.97 μmol, 51.08% yield, 98.867% purity, FA salt) was obtained as a white solid. LCMS (Method D): Rt=0.299 min, [M+H]⁺=844.6. SFC: Rt=1.377 min, 1.474 min. ¹H NMR (400 MHz, METHANOL-d4) δ=8.43 (s, 2H), 8.37 (d, J=7.8 Hz, 1H), 7.98-7.93 (m, 1H), 7.91-7.81 (m, 2H), 7.53-7.47 (m, 1H), 7.40-7.32 (m, 1H), 7.21-7.13 (m, 1H), 4.39 (s, 2H), 4.22 (s, 1H), 3.88-3.59 (m, 14H), 3.56-3.46 (m, 3H), 3.39 (s, 1H), 3.12-3.00 (m, 2H), 2.91-2.66 (m, 4H), 2.43-2.34 (m, 2H), 2.02-1.91 (m, 3H), 1.83 (d, J=10.0 Hz, 6H), 1.77-1.61 (m, 9H), 1.35-1.09 (m, 6H). ¹⁹F NMR (376 MHz, METHANOL-d4) δ=−120.792.

### Step 2: Synthesis of I-675.

To a solution of Intermediate 1-3 (80 mg, 89.88 μmol, 1 eq, FA salt) in THF (1 mL) was added 3,4,6,7,8,9-hexahydro-2H-pyrimido[1,2-a]pyrimidine (37.53 mg, 269.65 μmol, 3 eq). The mixture was stirred at 60° C. for 12 hr. The reaction mixture was diluted with H₂O (2 mL) and extracted with EA (5 mL*3). The combined organic layers were concentrated under reduced pressure to give a residue. The crude product was purified by reversed-phase chromatography (0.1% FA condition), the eluent was concentrated and lyophilized to give the desired product. I-675 (21.17 mg, 23.50 μmol, 26.15% yield, 95.259% purity, FA salt) was obtained as a white solid. LCMS (Method D): Rt=0.416 min, [M+H]⁺=812.5. SFC: Rt=1.414 min. ¹H NMR (400 MHz, METHANOL-d4) δ=8.42 (s, 1H), 8.37 (d, J=7.6 Hz, 1H), 7.99-7.91 (m, 1H), 7.90-7.80 (m, 2H), 7.55-7.46 (m, 1H), 7.40-7.32 (m, 1H), 7.22-7.11 (m, 1H), 5.27 (d, J=10.8 Hz, 1H), 4.39 (s, 2H), 4.11-3.87 (m, 2H), 3.84-3.64 (m, 7H), 3.61-3.42 (m, 4H), 3.38-3.33 (m, 2H), 3.28-3.11 (m, 5H), 3.04-2.82 (m, 2H), 2.55-2.33 (m, 2H), 2.11-1.98 (m, 3H), 1.79 (s, 9H), 1.68 (d, J=11.6 Hz, 3H), 1.54 (d, J=11.2 Hz, 2H), 1.44-1.13 (m, 4H), 1.04-0.85 (m, 2H). ¹⁹F NMR (376 MHz, METHANOL-d4) δ=−120.755.

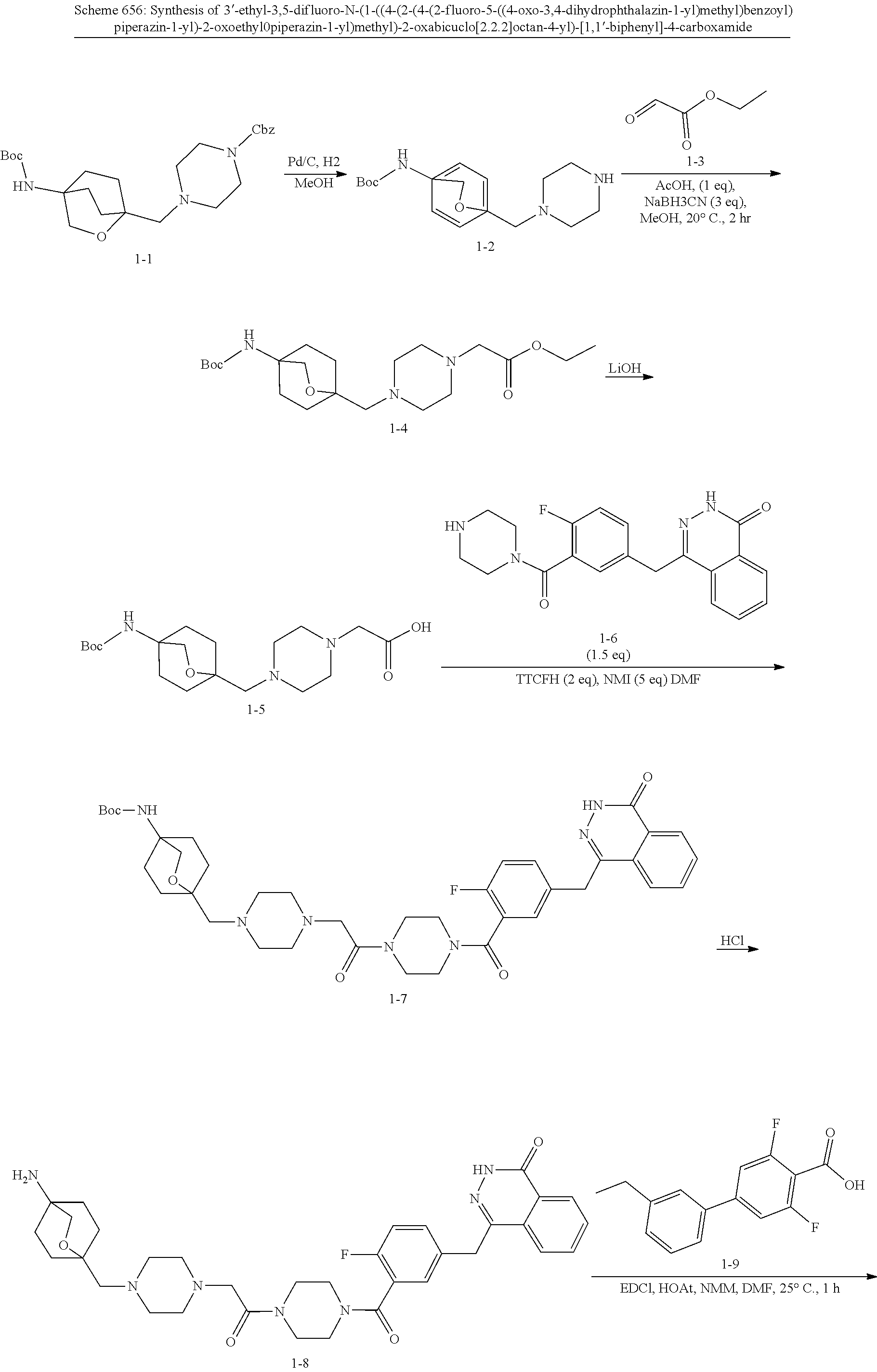

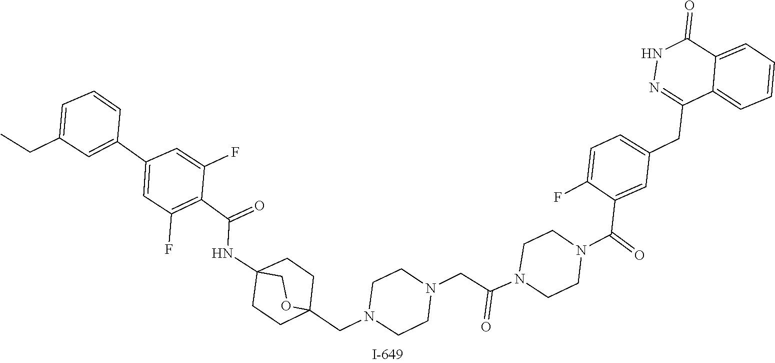 [SMILES: CCc1cccc(-c2cc(F)c(C(=O)NC34CCC(CN5CCN(CC(=O)N6CCN(C(=O)c7cc(Cc8n[nH]c(=O)c9ccccc89)ccc7F)CC6)CC5)(CC3)OC4)c(F)c2)c1]

### Step 1: Synthesis of Intermediate 1-2

A solution of Intermediate 1-1 (3.0 g, 9.22 mmol) in MeOH (30 mL) and THF (60 mL) was prepared. A fixed bed (volume=5 mL) was packed with granular catalyst 5% Pd/Al₂O₃ (2 g), and then heated to 50° C. The H₂ back pressure regulator was adjusted to 1.5 MPa. The previously prepared solution was pumped into the fixed bed at a flow rate of 0.3 mL/min, and the flow rate of H₂ was set to 20 mL/min. The reaction mixture was flowed through fixed bed in 3.3 min, leaving the reaction zone and then was collected by a container. The reaction mixture was concentrated under reduced pressure to give a residue. The product was used in the next step without purification. Intermediate 1-2 (2.0 g, 6.15 mmol, yield 66.67%) was obtained as a white solid. LCMS (Method E): Rt=0.370 min, [M+H]⁺=326.2.

### Step 2: Synthesis of Intermediate 1-4

A solution of Intermediate 1-2 (2 g, 6.15 mmol, 1 eq) and Intermediate 1-3 (6.27 g, 30.73 mmol, 5 eq) in MeOH (20 mL) and AcOH (369.04 mg, 6.15 mmol, 351.81 μL, 1 eq) was stirred at 20° C. for 0.5 hr. Then NaBH₃CN (1.16 g, 18.44 mmol, 3 eq) was added into the mixture. The mixture was stirred at 20° C. for 1.5 hr. The mixture was concentrated and the residue was purified by Prep-HPLC (column: Phenomenex Luna C18 150*25 mm*10 um; mobile phase: [water (FA)-ACN]; gradient: 10%-40% B over 9 min) and concentrated to remove MeCN and then lyophilized to afford the product. Intermediate 1-4 (2 g, 4.76 mmol, 77.50% yield, 98% purity) was obtained as a white solid. LCMS (Method E): Rt=0.398 min, [M+H]⁺=412.2.

### Step 3: Synthesis of Intermediate 1-5

To a solution of Intermediate 1-4 (500 mg, 1.21 mmol, 1 eq) in H₂O (2 mL), MeOH (2 mL), and THF (2 mL) was added LiOH·H₂O (152.95 mg, 3.64 mmol, 3 eq). The mixture was stirred at 25° C. for 1 hr. The reaction mixture was concentrated under reduced pressure to give a residue. The product was used for the next step without purification. Intermediate 1-5 (400 mg, 1.04 mmol, 85.85% yield, 100% purity) was obtained as a white solid. LCMS (Method E): Rt=0.375 min, [M+H]⁺=384.2.

### Step 4: Synthesis of Intermediate 1-7

To a solution of Intermediate 1-5 (100 mg, 260.77 μmol, 1 eq) and Intermediate 1-6 (143.31 mg, 391.15 μmol, 1.5 eq) in DMF (1 mL) was added TCFH (146.33 mg, 521.54 μmol, 2 eq) and NMI (107.05 mg, 1.30 mmol, 103.93 μL, 5 eq). The mixture was stirred at 25° C. for 1 hr. The reaction mixture was extracted with ethyl acetate (4 mL*3). The organic phase was dried over anhydrous Na₂SO₄, filtered and concentrated under reduced pressure to give the crude product. The crude product was purified by flash silica gel chromatography (ISCO; 12 g SepaFlash Silica Flash Column, Eluent of 0˜100% @Ethyl acetate: Methanol 40 mL/min). Then the product was concentrated under reduced pressure to give a residue. Intermediate 1-7 (120 mg, 126.25 μmol, 48.42% yield, 77% purity) was obtained as white oil. LCMS (Method E): Rt=0.433 min, [M+H]⁺=732.5.

### Step 5: Synthesis of Intermediate 1-8

To a solution of Intermediate 1-7 (120 mg, 163.97 μmol, 1 eq) in DCM (1.5 mL) was added HCl/dioxane (2 M, 81.98 μL, 1 eq). The mixture was stirred at 25° C. for 0.5 hr. The reaction mixture was concentrated under reduced pressure to give a residue. The product was used in the next step without purification. Intermediate 1-9 (100 mg, 149.66 μmol, 91.27% yield, HCl salt) was obtained as a white solid. LCMS (Method E): Rt=0.361 min, [M+H]⁺=632.3.

### Step 6: Synthesis of I-649

To a solution of Intermediate 1-8 (50 mg, 74.83 μmol, 1 eq, HCl salt) and Intermediate 1-9 (23.55 mg, 89.79 μmol, 1.2 eq) in DMF (0.5 mL) was added EDCI (71.72 mg, 374.14 μmol, 5 eq), NMM (75.69 mg, 748.28 μmol, 82.27 μL, 10 eq) and HOAt (30.55 mg, 224.48 μmol, 3 eq). The mixture was stirred at 25° C. for 0.5 hr. The reaction mixture was partitioned between DCM (5 mL) and water (5 mL) and then extracted with DCM (5 mL*2). The organic layers were dried over Na₂SO₄, filtered and concentrated under reduced pressure to give a residue. The residue was purified by Prep-HPLC (column: Waters Xbridge 150*25 mm*5 um; mobile phase: [water(NH₄HCO₃)-ACN]; gradient: 40%-70% B over 10 min) and concentrated to remove MeCN and then lyophilized to afford the product. I-649 (16.56 mg, 18.90 mol, 25.26% yield, 100% purity) was obtained as a white solid. LCMS (Method E): Rt=0.520 min, [M+H]⁺=876.5. ¹H NMR (400 MHz, METHANOL-d4) δ=8.38 (br d, J=8.0 Hz, 1H), 7.99-7.93 (m, 1H), 7.91-7.80 (m, 2H), 7.53-7.42 (m, 3H), 7.42-7.35 (m, 2H), 7.33-7.25 (m, 3H), 7.19-7.14 (m, 1H), 4.39 (s, 2H), 4.08 (s, 2H), 3.82-3.64 (m, 4H), 3.61-3.38 (m, 3H), 3.37-3.34 (m, 1H), 3.27-3.12 (m, 2H), 2.75-2.69 (m, 2H), 2.67-2.40 (m, 8H), 2.34-2.18 (m, 4H), 2.11-1.98 (m, 4H), 1.81 (s, 2H), 1.29-1.23 (m, 3H). ¹⁹F NMR (377 MHz, METHANOL-d4) δ=−115.90 (s, 2F), −120.77 (br d, J=20.2 Hz, 1F).

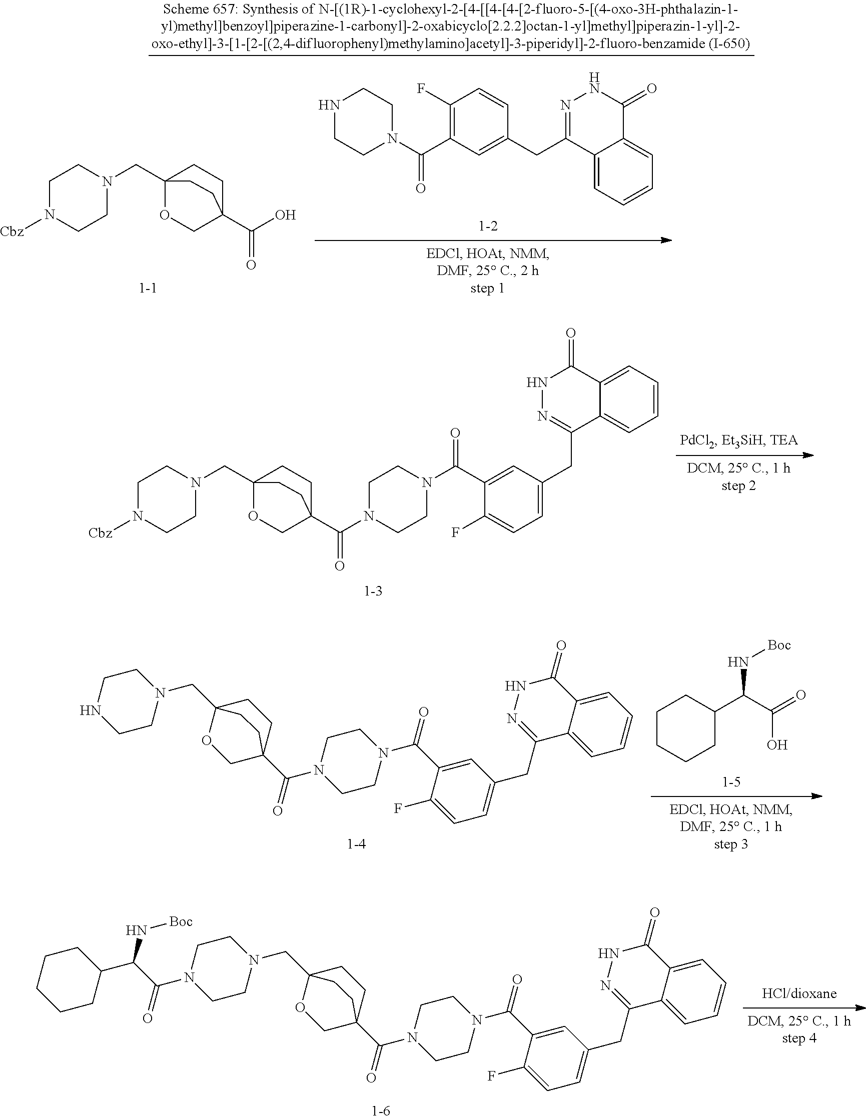

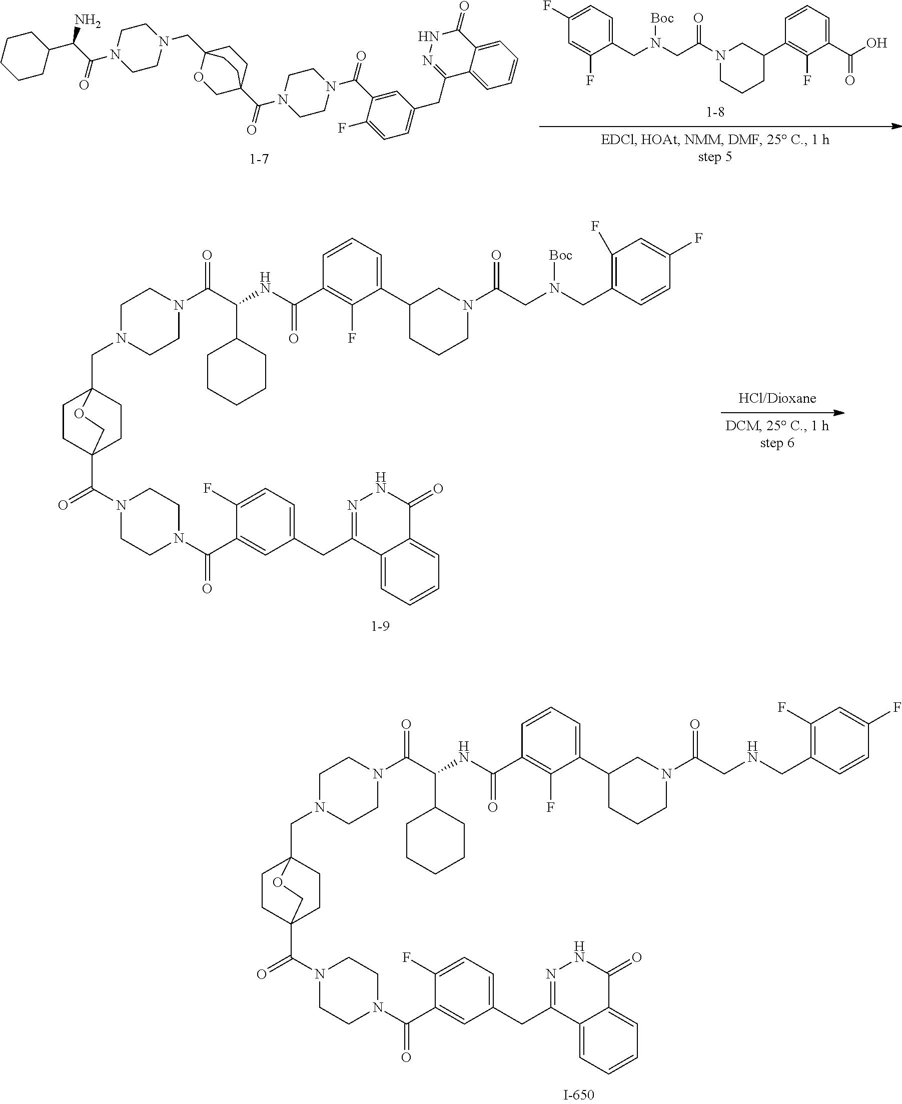 [SMILES: N[C@@H](C(=O)N1CCN(CC23CCC(C(=O)N4CCN(C(=O)c5cc(Cc6n[nH]c(=O)c7ccccc67)ccc5F)CC4)(CC2)CO3)CC1)C1CCCCC1 ; CC(C)(C)OC(=O)N(CC(=O)N1CCCC(c2cccc(C(=O)O)c2F)C1)Cc1ccc(F)cc1F ; CC(C)(C)OC(=O)N(CC(=O)N1CCCC(c2cccc(C(=O)N[C@@H](C(=O)N3CCN(CC45CCC(C(=O)N6CCN(C(=O)c7cc(Cc8n[nH]c(=O)c9ccccc89)ccc7F)CC6)(CC4)CO5)CC3)C3CCCCC3)c2F)C1)Cc1ccc(F)cc1F ; O=C(N[C@@H](C(=O)N1CCN(CC23CCC(C(=O)N4CCN(C(=O)c5cc(Cc6n[nH]c(=O)c7ccccc67)ccc5F)CC4)(CC2)CO3)CC1)C1CCCCC1)c1cccc(C2CCCN(C(=O)CNCc3ccc(F)cc3F)C2)c1F]

### Step 1: Synthesis of Intermediate 1-3

To a solution of Intermediate 1-1 (300 mg, 772.29 μmol, 1 eq) in DMF (3 mL) was added EDCI (74.02 mg, 386.14 μmol, 0.5 eq), NMM (781.15 mg, 7.72 mmol, 849.07 μL, 10 eq) and HOAt (315.35 mg, 2.32 mmol, 324.10 μL, 3 eq) followed addition of Intermediate 1-2 (339.55 mg, 926.75 μmol, 1.2 eq). The mixture was stirred at 25° C. for 2 hr. The residue was diluted with H₂O (5 mL) and extracted with EA (5 mL*3), dried over Na₂SO₄, filtered and concentrated under reduced pressure to give a residue. The residue was purified by flash silica gel chromatography (ISCO; 12 g SepaFlash Silica Flash Column, Eluent of 0-100% Dichloromethane/Methanol@60 mL/min). The eluent was concentrated under reduced pressure to give product. Intermediate 1-3 (560 mg, 760.01 μmol, 98.41% yield) was obtained as a yellow solid. LCMS (Method D): Rt=0.342 min [M+H]⁺=737.6.

### Step 2: Synthesis of Intermediate 1-4

To a solution of Intermediate 1-3 (500 mg, 678.58 μmol, 1 eq) in DCM (5 mL) was added Et₃SiH (946.84 mg, 8.14 mmol, 1.30 mL, 12 eq) and TEA (137.33 mg, 1.36 mmol, 188.90 μL, 2 eq), then the PdCl₂ (60.17 mg, 339.29 μmol, 0.5 eq) was added. The mixture was stirred at 25° C. for 1 hr. The mixture was filtered and diluted with H₂O (5 mL) and then extracted with DCM (5 mL*3) dried over Na₂SO₄, filtered and concentrated under reduced pressure to give Intermediate 1-4 (410 mg, crude) as a yellow oil, which was used in the next step without further purification. LCMS (Method D): Rt=0.274 min, [M+H]⁺=603.3.

### Step 3: Synthesis of Intermediate 1-6

To a solution of Intermediate 1-5 (204.94 mg, 796.42 μmol, 1.2 eq) in DMF (4 mL) was added EDCI (636.14 mg, 3.32 mmol, 5 eq), NMM (671.30 mg, 6.64 mmol, 729.67 μL, 10 eq), and HOAt (180.67 mg, 1.33 mmol, 185.68 μL, 2 eq) followed by Intermediate 1-4 (400 mg, 663.68 μmol, 1 eq). The mixture was stirred at 25° C. for 1 hr. The residue was diluted with H₂O (10 mL) and extracted with EA (5 mL*3), dried over Na₂SO₄, filtered and concentrated under reduced pressure to give a residue. The residue was purified by flash silica gel chromatography (ISCO; 16 g SepaFlash Silica Flash Column, Eluent of 0˜100% Dichloromethane/Methanol@60 mL/min). The eluent was concentrated under reduced pressure to give product. Intermediate 1-6 (550 mg, 607.48 μmol, 91.53% yield, 93% purity) was obtained as a white solid. LCMS (Method D): Rt=0.421 min, [M+H]⁺=842.6. SFC: Rt: 1.484 min.

### Step 4: Synthesis of Intermediate 1-7

To a solution of Intermediate 1-6 (470 mg, 558.19 μmol, 1 eq) in DCM (1.5 mL) was added HCl/dioxane (2 M, 3 mL, 10.75 eq). The mixture was stirred at 25° C. for 1 hr. The mixture was concentrated under reduced pressure to give Intermediate 1-7 (430 mg, 552.45 μmol, 98.97% yield, HCl salt) as a yellow solid, which was used in the next step without further purification. LCMS (Method D): Rt=0.312 min, [M+H]⁺=742.4.

### Step 5: Synthesis of Intermediate 1-9

To a solution of Intermediate 1-8 (352.29 mg, 695.52 μmol, 1.2 eq) in DMF (4.5 mL) was added EDCI (555.55 mg, 2.90 mmol, 5 eq), NMM (586.25 mg, 5.80 mmol, 637.23 μL, 10 eq) and HOAt (157.78 mg, 1.16 mmol, 162.16 μL, 2 eq), followed by Intermediate 1-7 (430 mg, 579.60 μmol, 1 eq). The mixture was stirred at 25° C. for 1 hr. The mixture was diluted with H₂O (5 mL) and extracted with EA (5 mL*3), dried over Na₂SO₄, filtered and concentrated under reduced pressure to give Intermediate 1-9 (600 mg, 487.65 μmol, 84.14% yield) as a yellow oil which was used in the next step without further purification. LCMS (Method D): Rt=0.458 min, [M+H]⁺=1231.6.

### Step 6: Synthesis of I-650

To a solution of Intermediate 1-9 (200 mg, 162.55 μmol, 1 eq) in DCM (0.5 mL) was added HCl/dioxane (2 M, 1.5 mL, 18.46 eq). The mixture was stirred at 25° C. for 1 hr. The residue was concentrated under reduced pressure to give a residue. The crude product was purified by reversed-phase chromatography (0.1% HCl). The eluent was concentrated under reduced pressure to remove ACN, then lyophilized to give I-650 (30 mg, 25.05 μmol, 15.41% yield, 97.404% purity, HCl salt) as a white solid. LCMS (Method D): Rt=0.390 min, [M+H]⁺=1130.8. SFC: Rt: 4.535 min, 5.523 min. ¹H NMR (400 MHz, METHANOL-d4) δ=8.41-8.34 (m, 1H), 7.98-7.94 (m, 1H), 7.90-7.81 (m, 2H), 7.68-7.60 (m, 1H), 7.58-7.46 (m, 3H), 7.39-7.34 (m, 1H), 7.30-7.25 (m, 1H), 7.20-7.08 (m, 3H), 4.63-4.50 (m, 2H), 4.39 (s, 2H), 4.36-4.28 (m, 3H), 4.24-4.13 (m, 2H), 4.05 (s, 2H), 3.76 (br s, 8H), 3.58 (br s, 3H), 3.28-3.02 (m, 6H), 2.95-2.74 (m, 1H), 2.33-2.23 (m, 2H), 2.10-1.88 (m, 9H), 1.87-1.60 (m, 9H), 1.41-1.01 (m, 6H). ¹⁹F NMR (377 MHz, METHANOL-d4) δ=−108.34-112.83-120.70-121.88.

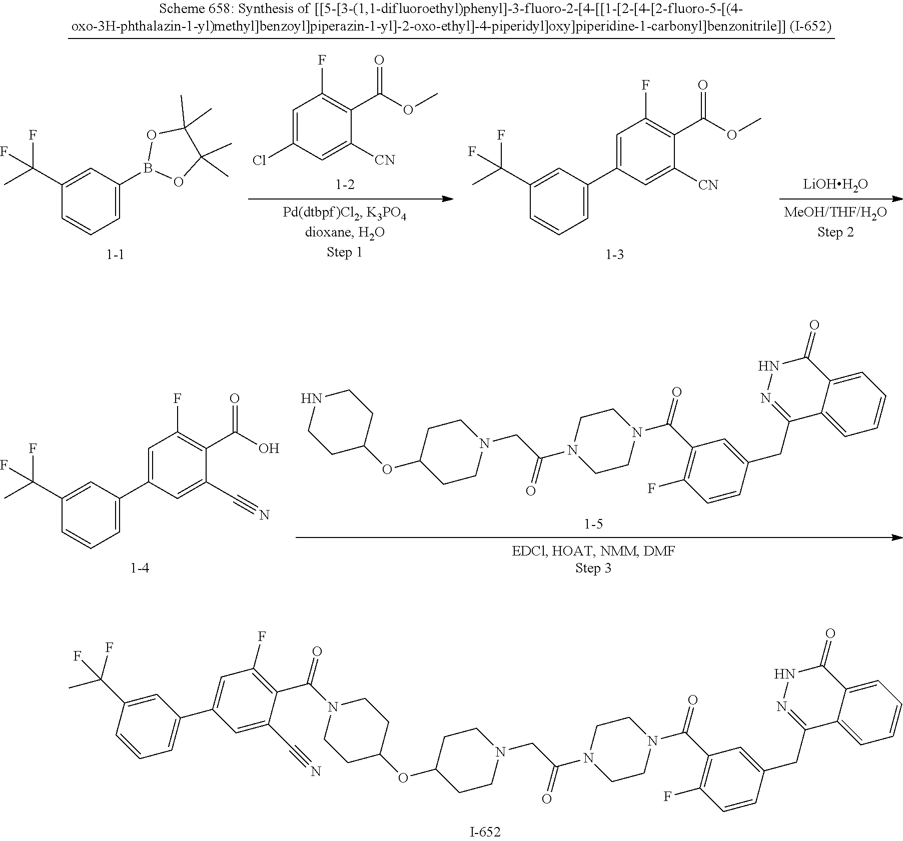

### Step 1: Synthesis of Intermediate 1-3

To a solution of Intermediate 1-2 (239.00 mg, 1.12 mmol, 1 eq) in dioxane (3 mL) and H₂O (0.6 mL) was added Intermediate 1-1 (300 mg, 1.12 mmol, 1 eq), K₃PO₄ (712.55 mg, 3.36 mmol, 3 eq), and ditert-butyl(cyclopenta-1,4-dien-1-yl)phosphane;dichloropalladium;iron (72.93 mg, 111.90 μmol, 0.1 eq). The mixture was stirred at 100° C. for 1 hr under N₂. The reaction mixture diluted with H₂O (3 mL) and extracted with EA (3 mL*2). The combined organic layers were dried over Na₂SO₄, filtered and concentrated under reduced pressure to give a residue. The residue was purified by flash silica gel chromatography (ISCO; 20 g SepaFlash Silica Flash Column, Eluent of 0˜15% Ethyl acetate/Petroleum ether gradient @60 mL/min). The eluent was concentrated under reduced pressure to give Intermediate 1-3 (330 mg, 978.80 μmol, 87.47% yield, 94.7% purity) as a yellow solid. LCMS (Method D): Rt: 0.477 min, [M+H]⁺=342.1. ¹H NMR (400 MHz, METHANOL-d4) δ=8.09-8.02 (m, 1H), 7.94-7.90 (m, 1H), 7.88 (s, 1H), 7.86-7.81 (m, 1H), 7.68-7.59 (m, 2H), 4.01 (s, 3H), 1.99 (t, J=18.4 Hz, 3H). ¹⁹F NMR (377 MHz, METHANOL-d4) δ=88.820, −109.168.

### Step 2: Synthesis of Intermediate 1-4

To a solution of Intermediate 1-3 (300 mg, 939.62 μmol, 1 eq) in MeOH (1 mL), THF (1 mL) and H₂O (1 mL) was added LiOH·H₂O (118.29 mg, 2.82 mmol, 3 eq). The mixture was stirred at 25° C. for 1 hr. The mixture was diluted with H₂O (3 mL) and HCl (1M) was added to adjust the pH to 4. The mixture was extracted with EA (3 mL*2). The combined organic layers were dried over Na₂SO₄, filtered and concentrated under reduced pressure to give Intermediate 1-3 (260 mg, 851.76 μmol, 90.65% yield, 85.5% purity) as yellow oil. LCMS (Method D): Rt: 0.424 min, [M+H]⁺=306.1.

### Step 3: Synthesis of I-652

To a solution of Intermediate 1-4 (30 mg, 98.28 μmol, 1 eq) in DMF (0.5 mL) was added EDCI (56.52 mg, 294.84 μmol, 3 eq), HOAt (13.38 mg, 98.28 μmol, 13.75 μL, 1 eq), Intermediate 1-5 (61.64 mg, 98.28 μmol, 1 eq, HCl salt) and NMM (49.70 mg, 491.40 μmol, 54.03 μL, 5 eq). The mixture was stirred at 25° C. for 1 hr. The mixture was diluted with H₂O (1 mL) and extracted with EA (1 mL*2). The combined organic layers were dried over Na₂SO₄, filtered and concentrated under reduced pressure to give a residue. The residue was purified by prep-HPLC (column: CD01-Phenomenex luna C18 150*25*10 um; mobile phase: [water(FA)-ACN]; gradient: 23%-53% B over 10 min). The eluent was concentrated under reduced pressure to remove ACN and then lyophilized to give I-652 (30.91 mg, 33.19 μmol, 33.77% yield, 99.2% purity, FA salt) as a white solid. LCMS (Method D): Rt: 0.406 min, [M+H]⁺=878.4. ¹H NMR (400 MHz, METHANOL-d4) δ=8.45 (s, 1H), 8.39-8.33 (m, 1H), 8.04 (s, 1H), 7.96-7.79 (m, 6H), 7.68-7.58 (m, 2H), 7.53-7.46 (m, 1H), 7.40-7.34 (m, 1H), 7.20-7.13 (m, 1H), 4.38 (s, 2H), 4.19-3.94 (m, 1H), 3.89-3.43 (m, 13H), 3.35 (s, 1H), 3.27 (s, 1H), 3.16-2.95 (m, 2H), 2.84-2.60 (m, 2H), 2.04-1.93 (m, 6H), 1.91-1.60 (m, 5H). ¹⁹F NMR (377 MHz, METHANOL-d4) δ=−88.760, −114.602, −120.757.

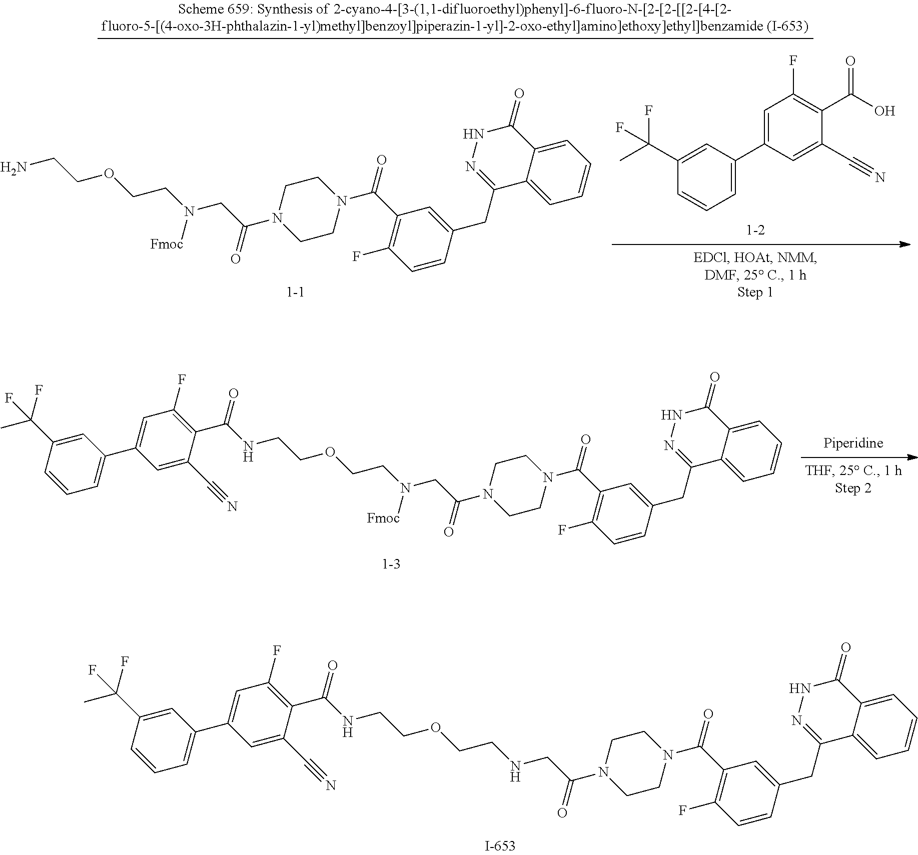

### Step 1: Synthesis of Intermediate 1-3

To a solution of Intermediate 1-1 (100 mg, 130.00 μmol, 1 eq, HCl salt) in DMF (1 mL) was added Intermediate 1-2 (39.68 mg, 130.00 μmol, 1 eq), EDCI (74.76 mg, 389.99 μmol, 3 eq), HOAt (17.69 mg, 130.00 μmol, 18.18 μL, 1 eq) and NMM (65.74 mg, 649.98 μmol, 71.46 μL, 5 eq). The mixture was stirred at 25° C. for 2 hrs. The reaction mixture was diluted with H₂O (2 mL) and extracted with EA (3 mL*2). The combined organic layers were washed with brine (2 mL*1), dried over Na₂SO₄, filtered and concentrated under reduced pressure to give a residue. The crude product was purified by reversed-phase chromatography (neutral condition). The eluent was concentrated under reduced pressure to remove ACN and then lyophilized to give Intermediate 1-3 (60 mg, 58.25 μmol, 44.81% yield, 99.032% purity) as a yellow solid. LCMS (Method D): Rt=0.732 min, [M+H]⁺=1020.2.

### Step 2: Synthesis of I-653

To a solution of Intermediate 1-3 (60 mg, 58.82 μmol, 1 eq) in THF (96 L) was added piperidine (10.35 mg, 121.51 μmol, 12.00 μL, 2.07 eq). The mixture was stirred at 25° C. for 1 hr. The reaction mixture was diluted with H₂O (0.5 mL) and extracted with EA (2 mL*2). The combined organic layers were dried over Na₂SO₄, filtered and concentrated under reduced pressure to give a residue. The crude product was purified by reversed-phase chromatography (Neutral condition). The eluent was concentrated under reduced pressure to remove ACN and then lyophilized to give the product. Then the crude product was purified by prep-HPLC (column: CD07-Daisogel SP-100-8-ODS-PK 150*25*10 um; mobile phase: [water (NH₄HCO₃)-ACN]; gradient: 29%-599% B over 11 min). The eluent was concentrated under reduced pressure to remove ACN and then lyophilized to give I-653 (7.78 mg, 9.73 μmol, 16.54% yield, 99.793% purity) as a white solid. LCMS (Method D): Rt=0.632 min, [M+H]⁺=798.5. ¹H NMR (400 MHz, METHANOL-d₄) δ=8.40-8.27 (m, 1H), 8.27-8.06 (m, 1H), 8.00-7.74 (m, 5H), 7.74-7.53 (m, 3H), 7.52-7.44 (m, 1H), 7.41-7.31 (m, 1H), 7.20-7.10 (m, 1H), 4.41-4.33 (m, 2H), 4.06-3.94 (m, 2H), 3.83-3.67 (m, 4H), 3.64-3.49 (m, 5H), 3.48-3.38 (m, 3H), 3.37-3.32 (m, 1H), 3.28-3.18 (m, 1H), 2.82-2.68 (m, 2H), 2.08-1.88 (m, 3H). ¹⁹F NMR (377 MHz, MeOD-d4) δ=−88.708, −117.148, −120.704.

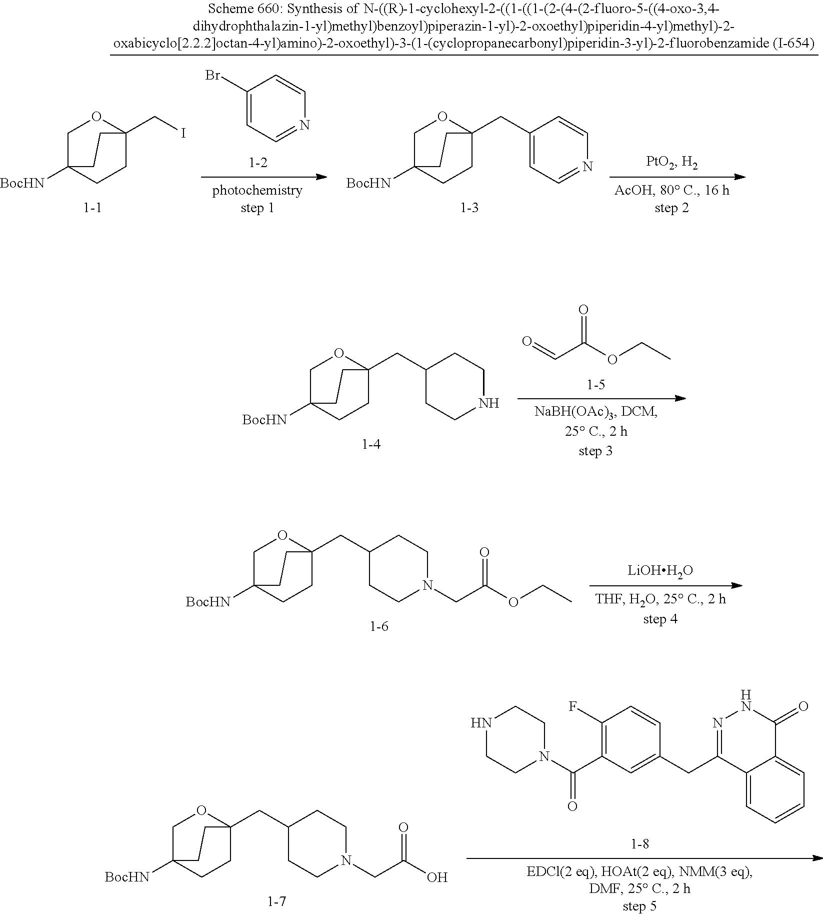

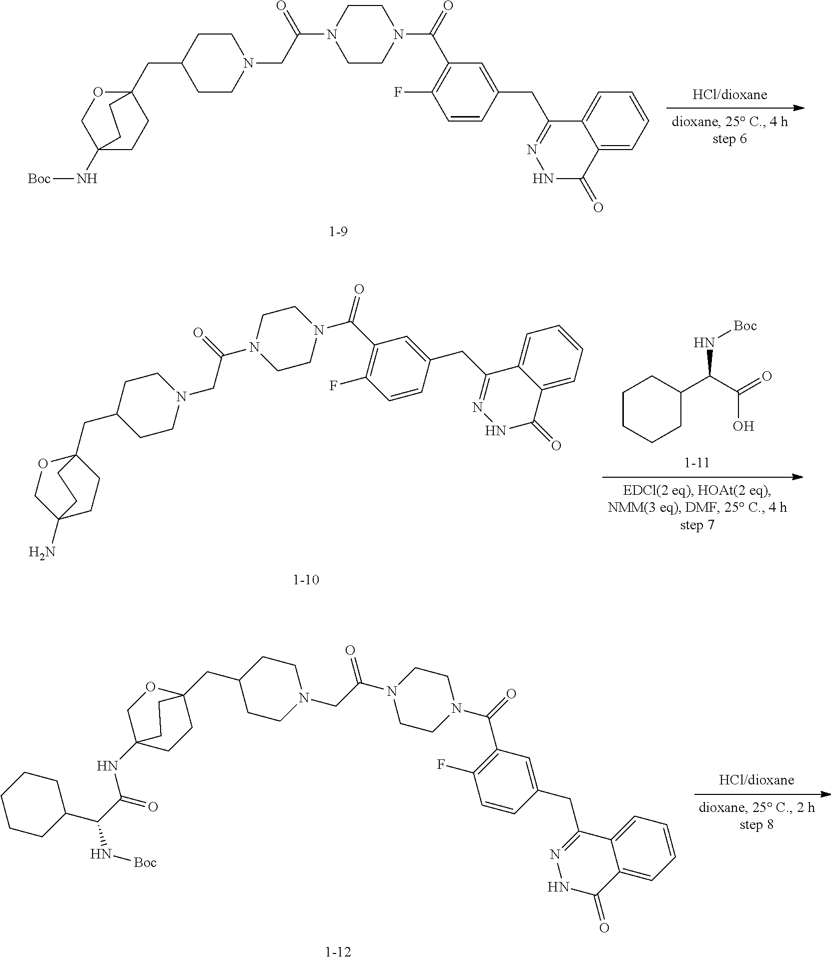 [SMILES: CC(C)(C)OC(=O)NC12CCC(CC3CCN(CC(=O)N4CCN(C(=O)c5cc(Cc6n[nH]c(=O)c7ccccc67)ccc5F)CC4)CC3)(CC1)OC2 ; "HCl/dioxane" ; NC12CCC(CC3CCN(CC(=O)N4CCN(C(=O)c5cc(Cc6n[nH]c(=O)c7ccccc67)ccc5F)CC4)CC3)(CC1)OC2 ; CC(C)(C)OC(=O)N[C@@H](C(=O)O)C1CCCCC1 ; CC(C)(C)OC(=O)N[C@@H](C(=O)NC12CCC(CC3CCN(CC(=O)N4CCN(C(=O)c5cc(Cc6n[nH]c(=O)c7ccccc67)ccc5F)CC4)CC3)(CC1)OC2)C1CCCCC1]

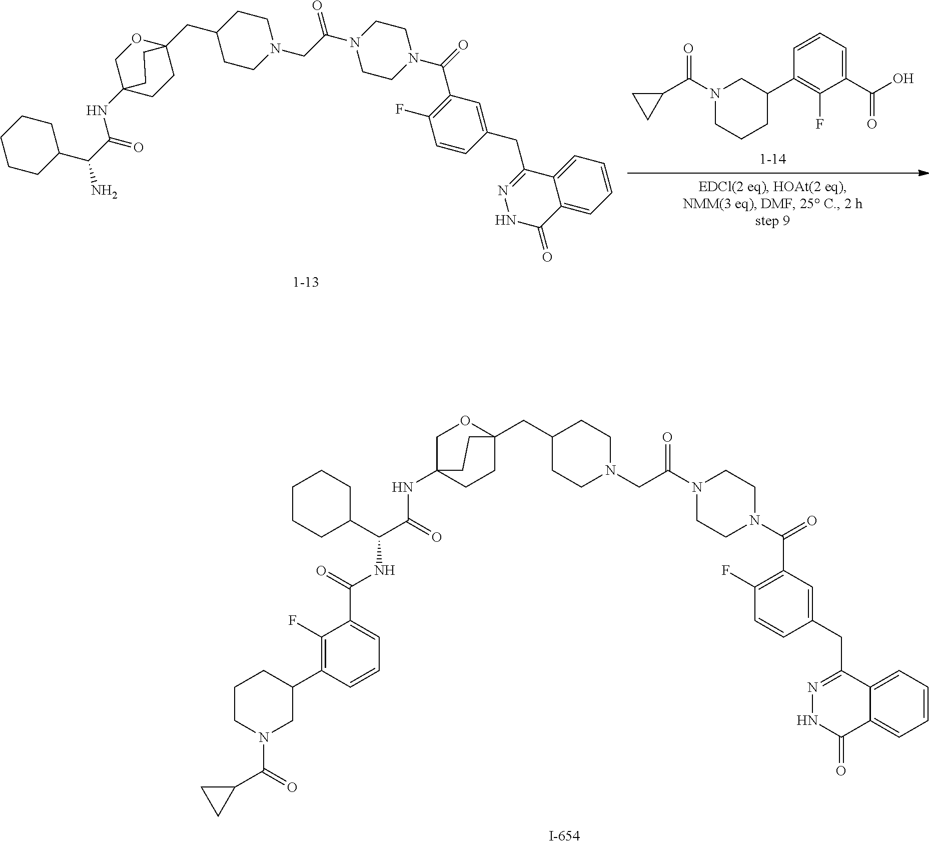 [SMILES: N[C@@H](C(=O)NC12CCC(CC3CCN(CC(=O)N4CCN(C(=O)c5cc(Cc6n[nH]c(=O)c7ccccc67)ccc5F)CC4)CC3)(CC1)OC2)C1CCCCC1 ; O=C(O)c1cccc(C2CCCN(C(=O)C3CC3)C2)c1F ; O=C(N[C@@H](C(=O)NC12CCC(CC3CCN(CC(=O)N4CCN(C(=O)c5cc(Cc6n[nH]c(=O)c7ccccc67)ccc5F)CC4)CC3)(CC1)OC2)C1CCCCC1)c1cccc(C2CCCN(C(=O)C3CC3)C2)c1F]

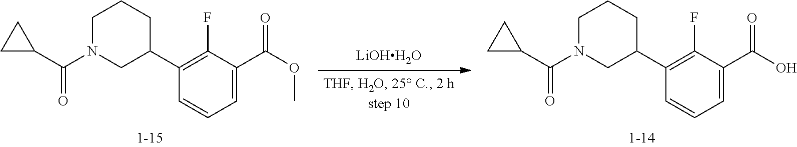 [SMILES: COC(=O)c1cccc(C2CCCN(C(=O)C3CC3)C2)c1F ; "LiOH·H2O" ; O=C(O)c1cccc(C2CCCN(C(=O)C3CC3)C2)c1F]

### Step 1: Synthesis of Intermediate 1-3

A mixture of Intermediate 1-2 (330.96 mg, 2.09 mmol, 1 eq), Intermediate 1-1 (1 g, 2.72 mmol, 1.3 eq), bis[3,5-difluoro-2-[5-(trifluoromethyl)-2-pyridyl]phenyl]iridium (1+); 4-tert-butyl-2-(4-tert-butyl-2-pyridyl)pyridine; hexafluorophosphate (23.50 mg, 20.95 μmol, 0.01 eq), 4-tert-butyl-2-(4-tert-butyl-2-pyridyl)pyridine; dichloronickel (4.17 mg, 10.47 μmol, 0.005 eq), bis(trimethylsilyl)silyl-trimethyl-silane (520.88 mg, 2.09 mmol, 646.25 μL, 1 eq) and Na₂CO₃ (444.04 mg, 4.19 mmol, 2 eq) in DME (10 mL) was degassed and purged with N₂, and then the mixture was stirred at 25° C. for 16 hr under irradiation by a 455 nm blue LED. The reaction mixture was diluted with water (30 mL) and extracted with EA (20 mL*3). The combined organic layers were dried over Na₂SO₄, filtered and concentrated under reduced pressure to give a residue. The residue was purified by flash silica gel chromatography (ISCO; 80 g SepaFlash Silica Flash Column, Eluent of 0˜100% Ethyl acetate/Petroleum ether gradient @100 mL/min). Intermediate 1-3 (0.3 g, 876.23 μmol, 41.83% yield, 93% purity) was obtained as a green solid. LCMS (Method E): Rt-0.400 min, [M+H]⁺=319.1. ¹H NMR (400 MHz, CHLOROFORM-d) δ=8.49 (d, J=5.6 Hz, 2H), 7.12 (d, J=5.6 Hz, 2H), 4.25 (br s, 1H), 3.92 (s, 2H), 2.67 (s, 2H), 2.05-1.99 (m, 2H), 1.88-1.76 (m, 4H), 1.69-1.59 (m, 2H), 1.42 (s, 9H).

### Step 2: Synthesis of Intermediate 1-4

To a solution of Intermediate 1-3 (0.2 g, 628.12 μmol, 1 eq) in AcOH (2 mL) was added PtO₂ (50.00 mg, 220.19 μmol, 3.51e−1 eq) under N₂ atmosphere. The suspension was degassed and purged with H₂ 3 times. The mixture was stirred under H₂ (50 Psi) at 80° C. for 16 hr. The mixture was filtered through a pad of Celite and washed with MeOH (10 mL*3), the filtrate was concentrated under vacuum to give a residue. Intermediate 1-4 (380 mg, crude, HOAc salt) was obtained as a green oil. LCMS (Method E): Rt=0.390 min, [M+H]⁺=325.2.

### Step 3: Synthesis of Intermediate 1-6

A solution of Intermediate 1-4 (380 mg, 988.27 μmol, 1 eq, HOAc salt) and Intermediate 1-5 (504.46 mg, 2.47 mmol, 2.5 eq) in DCM (4 mL) was stirred at 25° C. for 1 hr. Then NaBH(OAc)₃ (628.37 mg, 2.96 mmol, 3 eq) was added into the mixture. The mixture was stirred at 25° C. for 1 hr. The reaction mixture was diluted with water (10 mL), then extracted with DCM (10 ml*3). The combined organic layers were dried over Na₂SO₄, filtered and concentrated under reduced pressure to give a residue. Intermediate 1-6 (300 mg, crude) was obtained as a colorless oil. LCMS (Method E): Rt=0.521 min, [M+H]⁺=411.2. ¹H NMR (400 MHz, CHLOROFORM-d) δ=4.21-4.17 (m, 2H), 4.16 (s, 2H), 3.89 (s, 2H), 2.91 (d, J=11.2 Hz, 2H), 2.23-2.15 (m, 2H), 2.07-2.01 (m, 2H), 1.95-1.79 (m, 4H), 1.72 (d, J=12.0 Hz, 2H), 1.68-1.59 (m, 2H), 1.43 (s, 9H), 1.36-1.28 (m, 9H).

### Step 4: Synthesis of Intermediate 1-7

To a solution of Intermediate 1-6 (300 mg, 730.73 μmol, 1 eq) in THF (1.5 mL) and H₂O (1.5 mL) was added LiOH·H₂O (76.66 mg, 1.83 mmol, 2.5 eq). The mixture was stirred at 25° C. for 2 hr. The reaction mixture was concentrated under reduced pressure to remove THF. The residue was purified by reversed phase chromatography (26.8*125 mm, 80 g of XB—C18, 20-40 μm, 120 Å) Mobile phase: A for H₂O (0.1% FA v/v) and B for acetonitrile; Gradient: B 0%-100% in 15 min; Flow rate: 40 mL/min; Column temperature: R.T. Wavelength: 220 nm/254 nm). Intermediate 1-7 (100 mg, crude) was obtained as a white solid. LCMS (Method E): Rt=0.407 min, [M+H]⁺=383.2. ¹H NMR (400 MHz, DMSO-d₆) δ=6.55 (s, 1H), 3.71 (s, 2H), 3.25-3.14 (m, 4H), 2.68-2.63 (m, 2H), 1.94-1.85 (m, 2H), 1.79-1.71 (m, 4H), 1.81-1.68 (m, 1H), 1.61-1.50 (m, 3H), 1.46-1.38 (m, 2H), 1.35 (s, 9H), 1.22 (d, J=6.0 Hz, 2H).

### Step 5: Synthesis of Intermediate 1-9

To a solution of Intermediate 1-7 (100 mg, 261.44 μmol, 1 eq) in DMF (1 mL) was added Intermediate 1-8 (143.68 mg, 392.16 μmol, 1.5 eq), EDCI (100.24 mg, 522.89 μmol, 2 eq), NMM (79.33 mg, 784.33 μmol, 86.23 μL, 3 eq) and HOAt (71.17 mg, 522.89 μmol, 73.15 μL, 2 eq). The mixture was stirred at 25° C. for 2 hr. The reaction mixture was diluted with water (10 mL), then filtered and the filter cake was dried under reduced pressure to give a residue. Intermediate 1-9 (100 mg, crude) was obtained as a white solid. LCMS (Method E): Rt=0.451 min, [M+H]⁺=731.4.

### Step 6: Synthesis of Intermediate 1-10

To a solution of Intermediate 1-9 (100 mg, 136.82 μmol, 1 eq) in dioxane (1 mL) was added HCl/dioxane (2 M, 1 mL, 14.62 eq). The mixture was stirred at 25° C. for 4 hr. The reaction mixture was concentrated under reduced pressure to remove dioxane. Intermediate 1-10 (90 mg, crude) was obtained as a white solid. LCMS (Method E): Rt=0.373 min, [M+H]⁺=631.4.

### Step 7: Synthesis of Intermediate 1-12

To a solution of Intermediate 1-10 (90 mg, 142.69 μmol, 1 eq) in DMF (1 mL) was added Intermediate 1-11 (44.06 mg, 171.22 μmol, 1.2 eq), EDCI (54.71 mg, 285.37 μmol, 2 eq), NMM (43.30 mg, 428.06 μmol, 47.06 μL, 3 eq) and HOAt (38.84 mg, 285.37 μmol, 39.92 μL, 2 eq). The mixture was stirred at 25° C. for 4 hr. The reaction mixture was diluted with water (10 mL), then filtered and the filter cake was dried under reduced pressure. The solid was purified by reversed phase chromatography (26.8*125 mm, 25 g of XB—C18, 20-40 μm, 120 Å) Mobile phase: A for H₂O (0.1% FA v/v) and B for acetonitrile; Gradient: B 0%-100% in 15 min; Flow rate: 40 mL/min; Column temperature: R.T. Wavelength: 220/254 nm). Intermediate 1-12 (40 mg, 45.97 μmol, 32.22% yield, 100% purity) was obtained as a whitesolid. LCMS (Method E): Rt=0.509 min, [M+H]⁺=870.7. SFC: Rt=1.651 min. ¹H NMR (400 MHz, CHLOROFORM-d) δ=10.64-10.43 (m, 1H), 10.17-9.94 (m, 1H), 8.49-8.41 (m, 1H), 8.13 (s, 1H), 7.83-7.69 (m, 3H), 7.34 (d, J=5.6 Hz, 2H), 7.08-7.03 (m, 1H), 5.71-5.48 (m, 1H), 5.15-4.90 (m, 1H), 4.28 (s, 2H), 4.04-3.91 (m, 2H), 3.70 (s, 4H), 3.61-3.54 (m, 2H), 3.43-3.26 (m, 2H), 3.21-2.99 (m, 2H), 2.18-2.07 (m, 2H), 2.00-1.83 (m, 8H), 1.77-1.61 (m, 12H), 1.44 (s, 9H), 1.37-1.31 (m, 2H), 1.29-1.20 (m, 2H), 1.17-1.08 (m, 1H), 1.04-0.90 (m, 2H). ¹⁹F NMR (377 MHz, CHLOROFORM-d) δ=−117.56 (s, 1F).

### Step 8: Synthesis of Intermediate 1-13

To a solution of Intermediate 1-12 (40 mg, 45.97 μmol, 1 eq) in dioxane (1 mL) was added HCl/dioxane (2 M, 1 mL, 43.50 eq). The mixture was stirred at 25° C. for 2 hr. The reaction mixture was concentrated under reduced pressure to remove dioxane. Intermediate 1-13 (20 mg, 24.68 μmol, 53.68% yield, 95% purity) was obtained as a white solid. LCMS (Method E): Rt=0.403 min, [M+H]⁺=770.5.

### Step 9: Synthesis of I-654

To a solution of Intermediate 1-13 (20 mg, 25.98 μmol, 1 eq) in DMF (1 mL) was added Intermediate 1-14 (11.35 mg, 38.96 μmol, 1.5 eq), NMM (7.88 mg, 77.93 μmol, 8.57 μL, 3 eq), EDCI (9.96 mg, 51.95 μmol, 2 eq) and HOAt (7.07 mg, 51.95 μmol, 7.27 μL, 2 eq). The mixture was stirred at 25° C. for 2 hr. The reaction mixture was diluted with water (10 mL), then filtered and the filter cake was dried under reduced pressure to give a residue. The residue was purified by reversed phase chromatography (26.8*125 mm, 40 g of XB—C18, 20-40 μm, 120 Å) Mobile phase: A for H₂O (0.1% FA v/v) and B for acetonitrile; Gradient: B 0%-100% in 15 min; Flow rate: 40 mL/min; Column temperature: R.T. Wavelength: 220 nm/254 nm). I-654 (5.83 mg, 5.08 μmol, 19.57% yield, 95% purity, FA salt) was obtained as a white solid. LCMS (Method E): Rt=0.500 min, [M/2+H]⁺=522.5. SFC: Rt=2.188 min, 2.806 min. ¹H NMR (400 MHz, METHANOL-d₄) δ=8.57-8.44 (m, 1H), 8.37 (d, J=7.6 Hz, 1H), 7.98-7.92 (m, 1H), 7.91-7.80 (m, 2H), 7.60-7.46 (m, 3H), 7.39-7.36 (m, 1H), 7.28-7.22 (m, 1H), 7.19-7.14 (m, 1H), 4.58 (d, J=6.0 Hz, 2H), 4.39 (s, 2H), 4.32 (s, 1H), 4.05-3.92 (m, 2H), 3.90-3.65 (m, 5H), 3.63-3.41 (m, 4H), 3.35 (s, 1H), 3.27-2.98 (m, 4H), 2.85-2.58 (m, 3H), 2.18-2.11 (m, 1H), 2.05-2.01 (m, 2H), 1.94 (br s, 8H), 1.83-1.65 (m, 9H), 1.47-1.21 (m, 7H), 1.21-1.10 (m, 3H), 0.94-0.74 (m, 5H). ¹⁹F NMR (377 MHz, METHANOL-d₄) δ=−120.70 (br d, J=24.7 Hz, 1F), −121.67-−121.98 (m, 1F).

### Step 10: Synthesis of Intermediate 1-14

To a solution of Intermediate 1-15 (130 mg, 425.75 μmol, 1 eq) in THF (0.5 mL) and H₂O (0.5 mL) was added LiOH·H₂O (53.60 mg, 1.28 mmol, 3 eq). The mixture was stirred at 25° C. for 2 hr. The reaction mixture was adjusted to pH=1 with 1M HCl, then extracted with EA (10 mL*3). The combined organic layers were dried over Na₂SO₄, filtered and concentrated under reduced pressure to give a residue. Intermediate 1-15 (120 mg, 407.80 μmol, 95.78% yield, 99% purity) was obtained as a white solid. LCMS (Method E): Rt=0.454 min, [M+H]⁺=292.2.

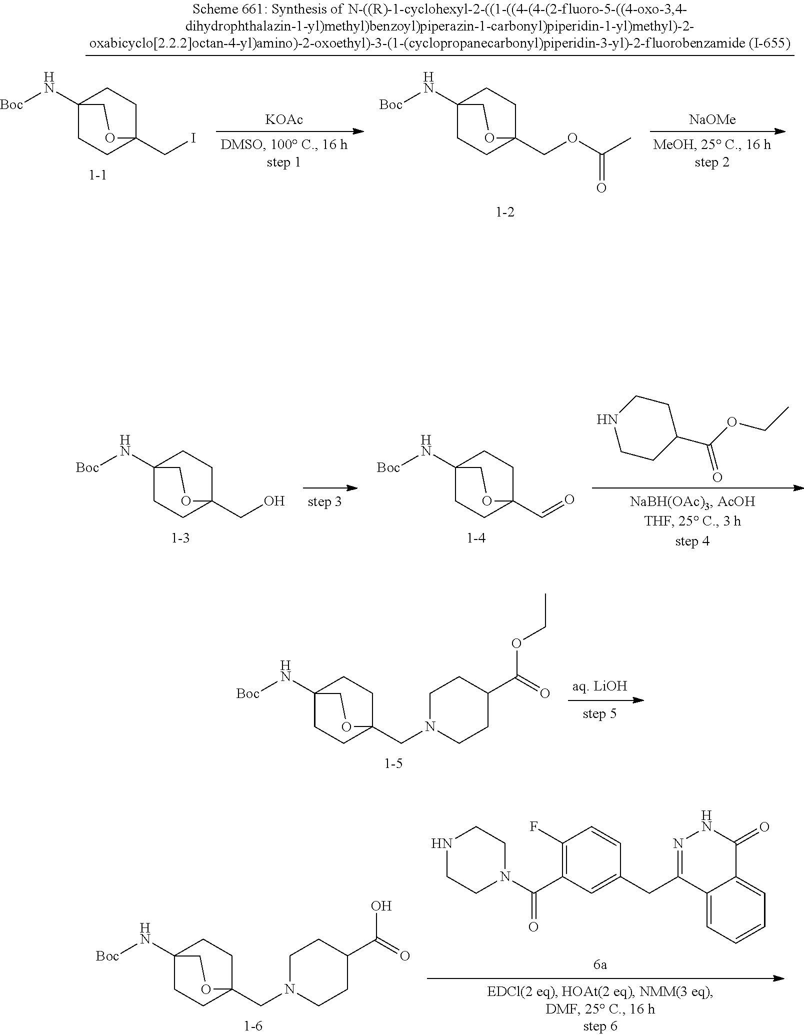

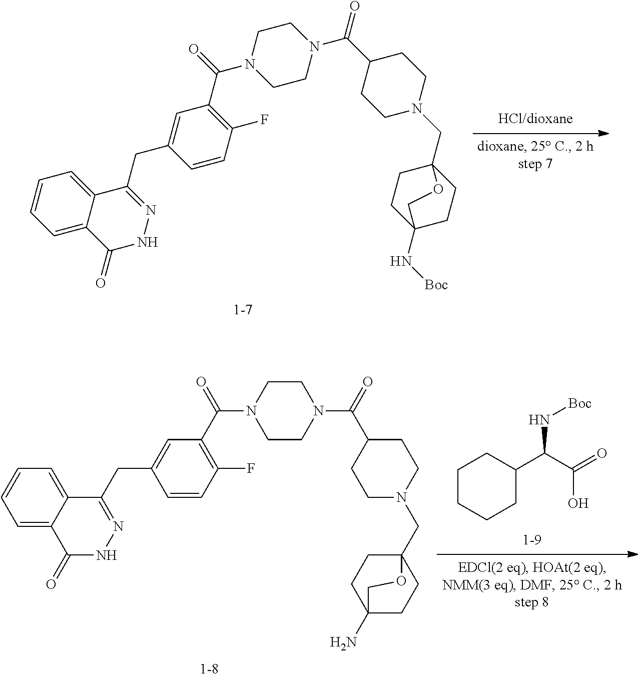

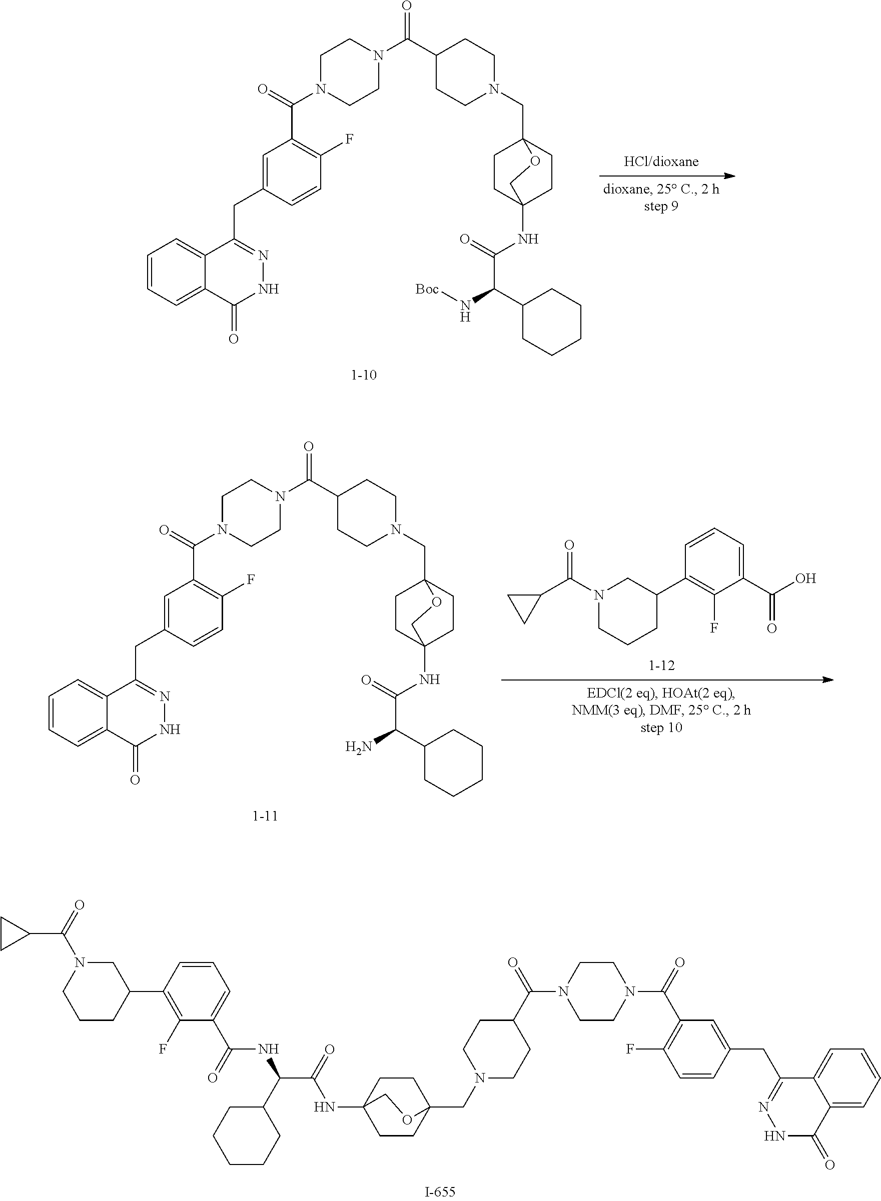 [SMILES: CC(C)(C)OC(=O)N[C@@H](C(=O)NC12CCC(CN3CCC(C(=O)N4CCN(C(=O)c5cc(Cc6n[nH]c(=O)c7ccccc67)ccc5F)CC4)CC3)(CC1)OC2)C1CCCCC1 ; N[C@@H](C(=O)NC12CCC(CN3CCC(C(=O)N4CCN(C(=O)c5cc(Cc6n[nH]c(=O)c7ccccc67)ccc5F)CC4)CC3)(CC1)OC2)C1CCCCC1 ; O=C(O)c1cccc(C2CCCN(C(=O)C3CC3)C2)c1F ; O=C(N[C@@H](C(=O)NC12CCC(CN3CCC(C(=O)N4CCN(C(=O)c5cc(Cc6n[nH]c(=O)c7ccccc67)ccc5F)CC4)CC3)(CC1)OC2)C1CCCCC1)c1cccc(C2CCCN(C(=O)C3CC3)C2)c1F]

### Step 1: Synthesis of Intermediate 1-2

To a solution of Intermediate 1-1 (8.5 g, 23.15 mmol, 1 eq) in DMSO (80 mL) was added KOAc (5.68 g, 57.87 mmol, 2.5 eq), and the mixture was stirred at 100° C. for 16 hr. The mixture was poured into water (200 mL) and extracted with EA (100 mL*2). The combined organic phase was dried over anhydrous Na₂SO₄, filtered and concentrated under vacuum. The residue was purified by flash silica gel chromatography (ISCO; 330 g SepaFlash Silica Flash Column, Eluent of 0˜30% Ethyl acetate/Petroleum ether gradient @100 mL/min). Intermediate 1-2 (2.5 g, 8.35 mmol, 36.08% yield) was obtained as a white solid. ¹H NMR (400 MHz, CHLOROFORM-d) δ=4.30 (br s, 1H), 3.96 (s, 2H), 3.93 (s, 2H), 2.15-2.05 (m, 5H), 2.03-1.91 (m, 2H), 1.91-1.79 (m, 2H), 1.65 (dt, J=4.1, 11.9 Hz, 2H), 1.42 (s, 9H). ¹H NMR (400 MHz, CHLOROFORM-d) δ=4.28 (br s, 1H), 3.97 (s, 2H), 3.16 (s, 2H), 2.16-1.99 (m, 4H), 1.95-1.75 (m, 4H), 1.43 (s, 9H).

### Step 2: Synthesis of Intermediate 1-3

To a solution of Intermediate 1-2 (2.5 g, 8.35 mmol, 1 eq) in MeOH (25 mL) was added NaOMe (5.4 M, 1.70 mL, 1.1 eq) dropwise at 0° C. under N₂. The mixture was stirred at 25° C. for 16 hr. The mixture was poured into water (100 mL) and extracted with EA (50 mL*2). The combined organic phase was dried over anhydrous Na₂SO₄, filtered and the filtrate was concentrated under vacuum to give a residue. The residue was used directly without further purification. Intermediate 1-3 (1.7 g, 6.61 mmol, 79.11% yield) was obtained as white solid. ¹H NMR (400 MHz, CHLOROFORM-d) δ=4.31 (br s, 1H), 3.97 (s, 2H), 3.39 (s, 2H), 2.17-2.06 (m, 2H), 2.05-1.97 (m, 2H), 1.87-1.80 (m, 2H), 1.59 (dt, J=3.9, 12.1 Hz, 2H).

### Step 3: Synthesis of Intermediate 1-4

To a solution of Intermediate 1-3 (500 mg, 1.96 mmol, 2.5 eq) in DCM (5 mL) was added ethyl piperidine-4-carboxylate (123.15 mg, 783.36 μmol, 120.74 μL, 1 eq) and AcOH (47.04 mg, 783.36 μmol, 44.84 μL, 1 eq). The mixture was stirred at 25° C. for 1 hr. Then NaBH(OAc)₃ (498.08 mg, 2.35 mmol, 3 eq) was added into the mixture. The mixture was stirred at 25° C. for 1 hr. The reaction mixture was diluted with water (10 mL) and extracted with DCM (10 mL*3). The combined organic layers were dried over Na₂SO₄, filtered and concentrated under reduced pressure to give a residue. The residue was used for the next step without further purification. Intermediate 1-4 (240 mg, crude) was obtained as a white solid. LCMS (Method E): Rt=0.401 min, (M+H=397.2). ¹H NMR (400 MHz, CHLOROFORM-d) δ=4.25 (s, 1H), 4.15-4.11 (m, 2H), 3.90 (s, 2H), 2.90 (d, J=11.6 Hz, 2H), 2.25-2.16 (m, 3H), 2.15-2.06 (m, 2H), 2.06-2.01 (m, 2H), 2.00-1.90 (m, 2H), 1.88-1.77 (m, 4H), 1.77-1.62 (m, 4H), 1.43 (s, 9H), 1.26-1.23 (m, 3H).

### Step 4: Synthesis of Intermediate 1-5

To a solution of Intermediate 1-4 (500 mg, 1.96 mmol, 2.5 eq) in DCM (5 mL) was added ethyl piperidine-4-carboxylate (123.15 mg, 783.36 μmol, 120.74 μL, 1 eq) and AcOH (47.04 mg, 783.36 μmol, 44.84 μL, 1 eq). The mixture was stirred at 25° C. for 1 hr. Then NaBH(OAc)₃ (498.08 mg, 2.35 mmol, 3 eq) was added into the mixture. The mixture was stirred at 25° C. for 1 hr. The reaction mixture was diluted with water (10 mL) and extracted with DCM (10 mL*3). The combined organic layers were dried over Na₂SO₄, filtered and concentrated under reduced pressure to give a residue. The residue was used in the next step without further purification. Intermediate 1-5 (240 mg, crude) was obtained as a white solid. LCMS (Method E): Rt=0.401 min, (M+H=397.2). ¹H NMR (400 MHz, CHLOROFORM-d) δ=4.25 (s, 1H), 4.15-4.11 (m, 2H), 3.90 (s, 2H), 2.90 (d, J=11.6 Hz, 2H), 2.25-2.16 (m, 3H), 2.15-2.06 (m, 2H), 2.06-2.01 (m, 2H), 2.00-1.90 (m, 2H), 1.88-1.77 (m, 4H), 1.77-1.62 (m, 4H), 1.43 (s, 9H), 1.26-1.23 (m, 3H).

### Step 5: Synthesis of Intermediate 1-6

To a solution of Intermediate 1-5 (240 mg, 605.27 μmol, 1 eq) in THF (1.2 mL) and H₂O (1.2 mL) was added LiOH·H₂O (76.20 mg, 1.82 mmol, 3 eq). The mixture was stirred at 25° C. for 1 hr. The reaction mixture was dried under reduced pressure to give a residue. The residue was used in the next step without further purification. Intermediate 1-6 (220 mg, 597.07 μmol, 98.65% yield) was obtained as a white solid. LCMS (Method B): Rt=0.447 min, (M+H=369.1).

### Step 6: Synthesis of Intermediate 1-7

To a solution of Intermediate 1-6 (200 mg, 542.79 μmol, 1 eq) in DMF (4 mL) was added Intermediate 1-6a (238.65 mg, 651.35 μmol, 1.2 eq), NMM (164.70 mg, 1.63 mmol, 179.03 μL, 3 eq), HOAt (147.76 mg, 1.09 mmol, 151.86 μL, 2 eq) and EDCI (208.11 mg, 1.09 mmol, 2 eq). The mixture was stirred at 40° C. for 16 hr. The reaction mixture was diluted with sat. NaCl:H₂O=1:3 (40 ml), and extracted with EA (10 mL*3). The combined organic layers were dried over Na₂SO₄, filtered and concentrated under reduced pressure to give a residue. The residue was purified by reversed phase chromatography (26.8*125 mm, 80 g of XB—C18, 20-40 μm, 120 Å) Mobile phase: A for H₂O (0.1% FA v/v) and B for acetonitrile; Gradient: B 5%-95% in 30 min; Flow rate: 40 mL/min; Column temperature: R.T. Wavelength: 220 nm/254 nm). Intermediate 1-7 (190 mg, 262.40 μmol, 48.34% yield, 99% purity) was obtained as a white solid. LCMS (Method E): Rt=0.438 min, (M+H=717.4). ¹H NMR (400 MHz, CHLOROFORM-d) δ=10.17-10.04 (m, 1H), 8.53-8.44 (m, 1H), 8.30 (s, 1H), 7.81-7.67 (m, 3H), 7.32 (d, J=4.4 Hz, 2H), 7.07-7.03 (m, 1H), 4.29 (s, 3H), 3.90 (s, 2H), 3.86-3.65 (m, 3H), 3.64-3.51 (m, 3H), 3.50-3.42 (m, 1H), 3.38-3.23 (m, 2H), 3.12-2.99 (m, 2H), 2.28-2.14 (m, 4H), 2.12-2.02 (m, 3H), 2.00-1.92 (m, 2H), 1.90-1.75 (m, 4H), 1.74-1.61 (m, 4H), 1.43 (s, 9H). ¹⁹F NMR (376 MHz, CHLOROFORM-d) δ=−116.92-−117.98 (m, 1F).

### Step 7: Synthesis of Intermediate 1-8

To a solution of Intermediate 1-7 (170 mg, 237.15 μmol, 1 eq) in dioxane (2 mL) was added HCl/dioxane (2 M, 2 mL, 16.87 eq). The mixture was stirred at 25° C. for 2 hr. The reaction mixture was concentrated under reduced pressure to give a residue. Intermediate 1-8 (300 mg, crude, HCl salt) was obtained as a white solid. LCMS (Method E): Rt=0.367 min, (M+H=617.4).

### Step 8: Synthesis of Intermediate 1-10

To a solution of Intermediate 1-8 (300 mg, 459.29 μmol, 1 eq, HCl salt) in DMF (3 mL) was added Intermediate 1-9 (177.28 mg, 688.93 μmol, 1.5 eq), NMM (139.37 mg, 1.38 mmol, 151.49 μL, 3 eq), EDCI (176.09 mg, 918.58 μmol, 2 eq) and HOAt (125.03 mg, 918.58 μmol, 128.50 μL, 2 eq). The mixture was stirred at 25° C. for 2 hr. The reaction mixture was combined with another lot of material for workup. The reaction mixture was diluted with water (30 mL) and extracted with EA (15 mL*3). The combined organic layers were washed with sat·NaCl (15 mL), dried over Na₂SO₄, filtered and concentrated under reduced pressure to give a residue. The residue was purified by reversed phase chromatography (26.8*125 mm, 40 g of XB—C18, 20-40 μm, 120 Å) Mobile phase: A for H₂O (0.1% FA v/v) and B for acetonitrile; Gradient: B 0%-100% in 15 min; Flow rate: 40 mL/min; Column temperature: R.T. Wavelength: 220 nm /254 nm). Intermediate 1-10 (180 mg, 170.32 μmol, 37.08% yield, 81% purity) was obtained as a white solid. LCMS (Method E): Rt=0.489 min, (M+H=856.5). ¹HNMR (400 MHz, CHLOROFORM-d) δ=10.25-10.14 (m, 1H), 8.51-8.43 (m, 1H), 7.84-7.67 (m, 3H), 7.32 (d, J=5.6 Hz, 2H), 7.09-7.01 (m, 1H), 5.55 (s, 1H), 5.09-4.91 (m, 1H), 4.29 (s, 2H), 4.03-3.91 (m, 2H), 3.80-3.66 (m, 3H), 3.60-3.53 (m, 2H), 3.51-3.43 (m, 1H), 3.38-3.23 (m, 2H), 3.11 (d, J=5.6 Hz, 2H), 2.47-2.35 (m, 4H), 2.20-2.09 (m, 2H), 2.06-1.96 (m, 2H), 1.94-1.80 (m, 4H), 1.79-1.60 (m, 10H), 1.44 (s, 9H), 1.29-1.08 (m, 3H), 1.05-0.87 (m, 2H). ¹⁹F NMR (376 MHz, DMSO-d6) δ=−73.42 (s, 1F), −118.07-−121.10 (m, 1F).

### Step 9: Synthesis of Intermediate 1-11

To a solution of Intermediate 1-10 (160 mg, 186.91 μmol, 1 eq) in dioxane (2 mL) was added HCl/dioxane (2 M, 2 mL, 21.40 eq). The mixture was stirred at 25° C. for 2 hr. The reaction mixture was concentrated under reduced pressure to give a residue. The residue was used in the next step without further purification. Intermediate 1-11 (160 mg, crude, HCl salt) was obtained as a white solid. LCMS (Method E):Rt=0.407 min, (M+H=756.5).

### Step 10: Synthesis of I-655

To a solution of Intermediate 1-11 (160 mg, 201.92 μmol, 1 eq, HCl salt) in DMF (2 mL) was added Intermediate 1-12 (70.59 mg, 242.31 μmol, 1.2 eq), NMM (61.27 mg, 605.77 μmol, 66.60 μL, 3 eq), EDCI (77.42 mg, 403.85 μmol, 2 eq) and HOAt (54.97 mg, 403.85 μmol, 56.49 μL, 2 eq). The mixture was stirred at 25° C. for 2 hr. The reaction mixture was combined with another lot of material for workup. The reaction mixture was diluted with sat·NaCl:H₂O (1:3) (40 mL) and extracted with EA (15 mL*3). The combined organic layers were washed with sat·NaCl (10 mL), dried over Na₂SO₄, filtered and concentrated under reduced pressure to give a residue. The residue was purified by reversed phase chromatography (26.8*125 mm, 80 g of XB—C18, 20-40 μm, 120 Å) Mobile phase: A for H₂O (0.1% FA v/v) and B for acetonitrile; Gradient: B 5%-95% in 15 min; Flow rate: 40 mL/min; Column temperature: R.T. Wavelength: 220 nm/254 nm). I-655 (75.74 mg, 73.59 μmol, 36.44% yield, 100% purity) was obtained as a white solid. LCMS (Method E): Rt=0.491 min, (M+H=1029.7). ¹H NMR (400 MHz, METHANOL-d4) δ=8.40-8.34 (m, 1H), 7.98-7.92 (m, 1H), 7.90-7.80 (m, 2H), 7.62-7.45 (m, 3H), 7.37 (d, J=0.8 Hz, 1H), 7.31-7.21 (m, 1H), 7.18-7.13 (m, 1H), 4.57 (br s, 1H), 4.38 (s, 3H), 4.33 (d, J=8.0 Hz, 1H), 3.98-3.96 (m, 2H), 3.82-3.62 (m, 4H), 3.56-3.44 (m, 2H), 3.28 (s, 2H), 3.14-2.96 (m, 3H), 2.88-2.69 (m, 1H), 2.68-2.48 (m, 1H), 2.27 (s, 2H), 2.22-2.08 (m, 4H), 2.07-1.53 (m, 22H), 1.32-1.00 (m, 5H), 0.95-0.73 (m, 4H). ¹⁹F NMR (377 MHz, METHANOL-d4) δ=−120.73 (br d, J=31.4 Hz, 1F), −121.65-−121.91 (m, 1F). SFC: Rt=1.841 min, 2.307 min, (ee %:ee %=40.9:59.1).

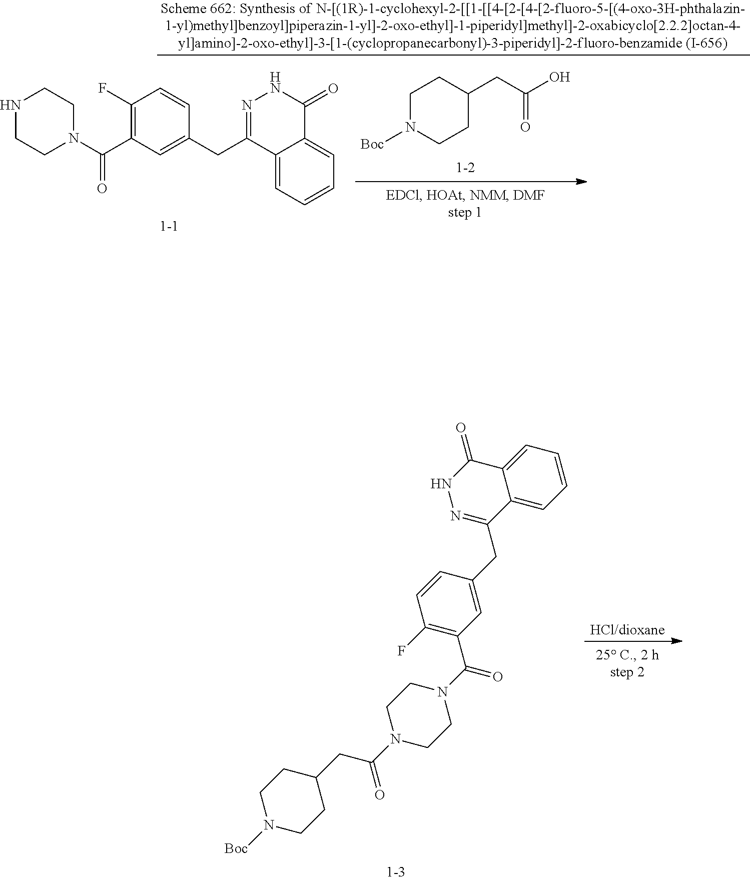 [SMILES: O=C(c1cc(Cc2n[nH]c(=O)c3ccccc23)ccc1F)N1CCNCC1 ; CC(C)(C)OC(=O)N1CCC(CC(=O)O)CC1 ; CC(C)(C)OC(=O)N1CCC(CC(=O)N2CCN(C(=O)c3cc(Cc4n[nH]c(=O)c5ccccc45)ccc3F)CC2)CC1 ; "HCl/dioxane"]

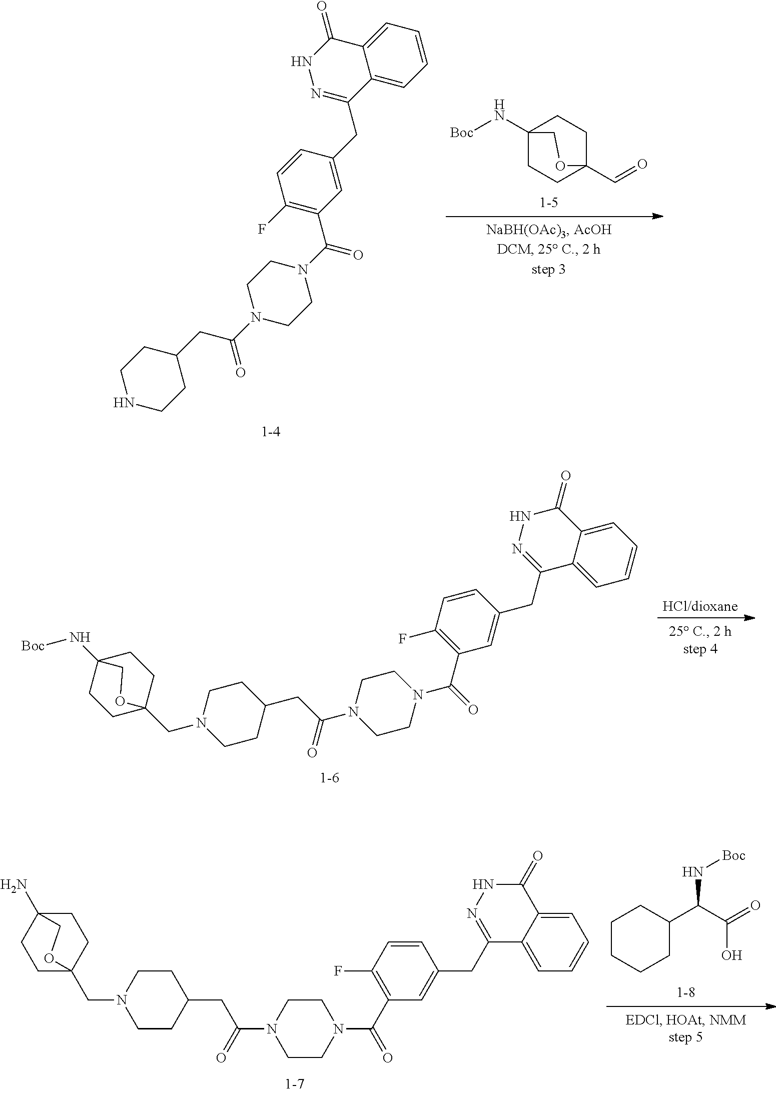

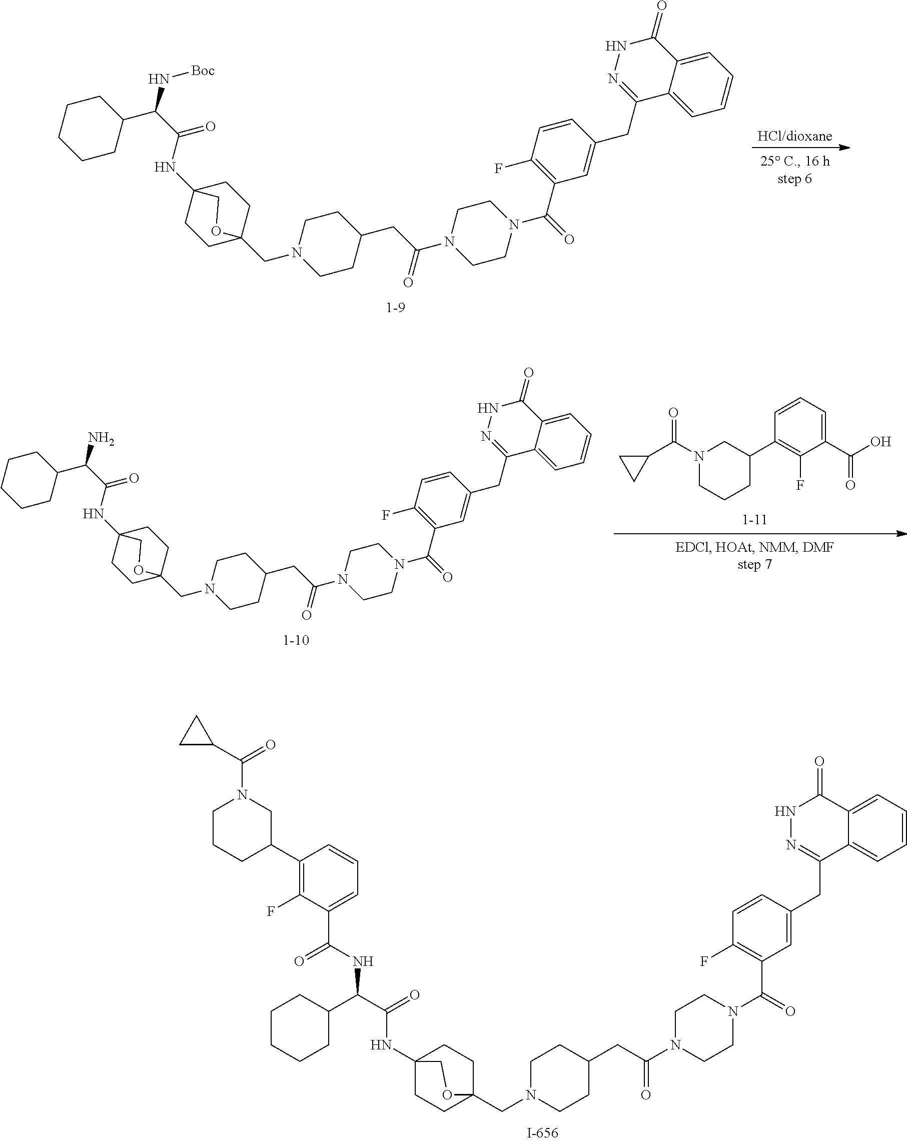

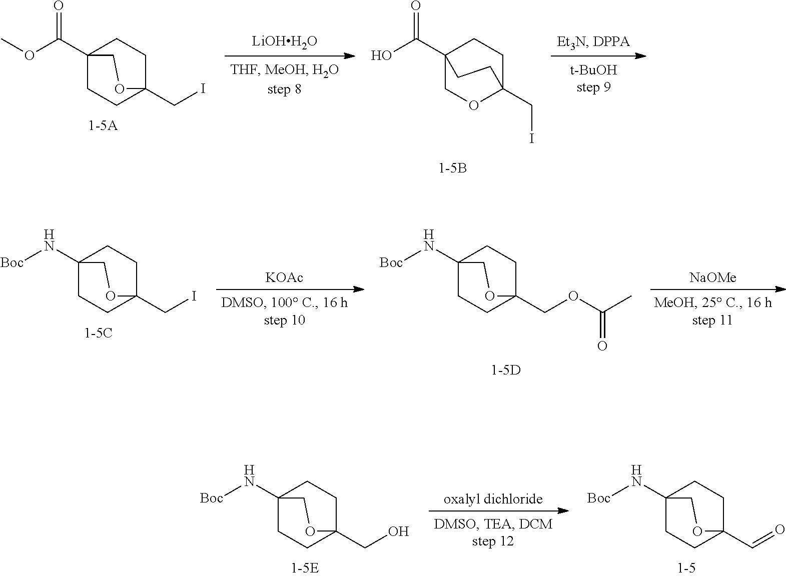

### Step 1: Synthesis of Intermediate 1-3

To a solution of Intermediate 1-2 (87.65 mg, 360.27 μmol, 1.1 eq) in DMF (2 mL) was added HOAt (89.16 mg, 655.04 μmol, 91.63 μL, 2 eq) and EDCI (125.57 mg, 655.04 μmol, 2 eq). The mixture was stirred at 25° C. for 30 min. Then Intermediate 1-1 (120 mg, 327.52 μmol, 1 eq) and NMM (99.38 mg, 982.56 μmol, 108.03 μL, 3 eq) were added and the mixture was stirred at 25° C. for 2 hr. The mixture was poured into saturated aq·NaCl (20 mL) and filtered. The filter cake was dried under vacuum. Intermediate 1-3 (190 mg, 321.12 μmol, 98.05% yield) was obtained as a yellow solid. LCMS (Method E): Rt=0.505 min, [M+H]⁺=592.4. ¹H NMR (400 MHz, CHLOROFORM-d) δ=10.14-9.99 (m, 1H), 8.52-8.44 (m, 1H), 7.81-7.70 (m, 3H), 7.40-7.30 (m, 2H), 7.11-7.01 (m, 1H), 4.29 (s, 2H), 4.16-4.03 (m, 3H), 3.88-3.66 (m, 3H), 3.57 (br d, J=4.3 Hz, 3H), 3.45 (br s, 1H), 3.38-3.17 (m, 2H), 2.80-2.67 (m, 2H), 2.31-2.19 (m, 2H), 2.05-1.97 (m, 1H), 1.74 (br d, J=10.3 Hz, 2H), 1.46 (s, 9H).

### Step 2: Synthesis of Intermediate 1-4

Intermediate 1-3 (190 mg, 321.12 μmol, 1 eq) in HCl/dioxane (2 M, 10 mL, 62.28 eq) was stirred at 25° C. for 2 hr. The mixture was concentrated under vacuum. The residue was used directly without further purification. Intermediate 1-4 (97 mg, 183.71 μmol, 57.21% yield, HCl salt) was obtained as a yellow solid. LCMS (Method E): Rt=0.383 min, [M+H]⁺=492.2. ¹H NMR (400 MHz, METHANOL-d₄) δ=8.37 (d, J=7.5 Hz, 1H), 8.00-7.92 (m, 1H), 7.91-7.80 (m, 2H), 7.53-7.44 (m, 1H), 7.37 (br t, J=5.3 Hz, 1H), 7.16 (t, J=8.9 Hz, 1H), 4.39 (s, 2H), 3.81-3.63 (m, 4H), 3.57-3.47 (m, 2H), 3.03 (br d, J=12.1 Hz, 2H), 2.70-2.54 (m, 2H), 2.42-2.27 (m, 2H), 1.98-1.82 (m, 1H), 1.80-1.67 (m, 2H), 1.22-1.14 (m, 1H).

### Step 3: Synthesis of Intermediate 1-6

To a solution of Intermediate 1-5 (50 mg, 195.84 μmol, 1 eq) and Intermediate 1-4 (96.27 mg, 182.32 μmol, 9.31e−1 eq, HCl salt) in DCM (2 mL) was added AcOH (117.61 g, 1.96 μmol, 1.12e−1 μL, 0.01 eq). The mixture was stirred at 25° C. for 1 hr. Then sodium; triacetoxyborohydride (124.52 mg, 587.52 μmol, 3 eq) was added and the mixture was stirred at 25° C. for 1 hr. The mixture was poured into aq·NH₄Cl (20 mL) and extracted with DCM (20 mL*2). The combined organic phase was dried over anhydrous Na₂SO₄, filtered and concentrated under vacuum. The residue was used directly without further purification. Intermediate 1-6 (60 mg, 72.24 μmol, 36.89% yield, 88% purity) was obtained as a yellow gum. LCMS (Method E): Rt=0.452 min, [M+H]⁺=731.5.

### Step 4: Synthesis of Intermediate 1-7

A mixture of Intermediate 1-6 (60 mg, 82.09 μmol, 1 eq) in HCl/dioxane (2 M, 5 mL, 121.81 eq) was stirred at 25° C. for 2 hr. The mixture was concentrated under vacuum. The residue was used directly without further purification. Intermediate 1-7 (60 mg, crude, HCl salt) was obtained as a white solid. LCMS (Method E): Rt=0.375 min, [M+H]⁺=631.4. ¹H NMR (400 MHz, METHANOL-d₄) δ=8.37 (d, J=7.8 Hz, 1H), 7.99-7.93 (m, 1H), 7.91-7.81 (m, 2H), 7.57-7.46 (m, 1H), 7.42-7.31 (m, 1H), 7.17 (t, J=9.1 Hz, 1H), 4.39 (s, 2H), 3.94 (s, 2H), 3.85-3.63 (m, 6H), 3.53-3.46 (m, 2H), 3.21 (s, 1H), 3.08 (brt, J=12.5 Hz, 1H), 2.46 (br d, J=5.8 Hz, 1H), 2.38 (br d, J=5.9 Hz, 1H), 2.26-2.14 (m, 2H), 2.07-1.90 (m, 12H), 1.79-1.63 (m, 2H).

### Step 5: Synthesis of Intermediate 1-9

To a solution of Intermediate 1-8 (25.45 mg, 98.92 μmol, 1.1 eq) in DMF (1 mL) was added HOAt (24.48 mg, 179.85 μmol, 25.16 μL, 2 eq) and EDCI (34.48 mg, 179.85 μmol, 2 eq). The mixture was stirred at 25° C. for 30 min. Then Intermediate 1-7 (60 mg, 89.93 μmol, 1 eq, HCl salt) and NMM (36.38 mg, 359.71 μmol, 39.55 μL, 4 eq) were added and the mixture was stirred at 25° C. for 2 hr. The mixture was poured into 10 mL of water and extracted with EA (10 mL*2). The combined organic phase was dried over anhydrous Na₂SO₄, filtered and concentrated in vacuum. The crude product was purified by reversed-phase HPLC (0.1% FA condition). Intermediate 1-9 (15 mg, 17.24 μmol, 19.17% yield) was obtained as a yellow solid. LCMS (Method E): Rt=0.492 min, [M+H]⁺=870.6. ¹H NMR (400 MHz, METHANOL-d₄) δ=8.54 (s, 1H), 8.37 (br d, J=7.4 Hz, 1H), 8.00-7.92 (m, 1H), 7.91-7.80 (m, 2H), 7.55-7.45 (m, 1H), 7.42-7.33 (m, 1H), 7.16 (t, J=8.8 Hz, 1H), 4.39 (s, 2H), 4.05-3.93 (m, 2H), 3.81-3.63 (m, 5H), 3.55-3.47 (m, 2H), 3.14-2.98 (m, 2H), 2.40-2.28 (m, 3H), 2.20-2.07 (m, 3H), 2.03-1.88 (m, 4H), 1.83-1.55 (m, 11H), 1.44 (s, 9H).

### Step 6: Synthesis of Intermediate 1-10

A mixture of Intermediate 1-9 (15 mg, 17.24 μmol, 1 eq) in HCl/dioxane (2 M, 5 mL) was stirred at 25° C. for 16 hr. The mixture was concentrated in vacuum. The residue was used directly without further purification. Intermediate 1-10 (16 mg, crude, HCl salt) was obtained as a white solid. LCMS (Method E): Rt=0.392 min, [M+H]⁺=770.4. ¹H NMR (400 MHz, DMSO-d₆) δ=12.60 (s, 1H), 8.28-8.22 (m, 1H), 8.20-8.12 (m, 1H), 8.07-8.02 (m, 2H), 7.97-7.94 (m, 1H), 7.91-7.81 (m, 2H), 7.52-7.43 (m, 1H), 7.42-7.30 (m, 1H), 7.28-7.21 (m, 1H), 4.36-4.31 (m, 2H), 4.04-3.95 (m, 1H), 3.89-3.80 (m, 1H), 3.70 (d, J=6.3 Hz, 1H), 3.59-3.50 (m, 8H), 3.22-3.12 (m, 7H), 1.77-1.50 (m, 13H).

### Step 7: Synthesis of I-656

To a solution of Intermediate 1-11 (8.67 mg, 29.76 μmol, 1.5 eq) in DMF (1 mL) was added HOAt (8.10 mg, 59.52 μmol, 8.33 μL, 3 eq) and EDCI (11.41 mg, 59.52 μmol, 3 eq). The mixture was stirred at 25° C. for 30 min. Then Intermediate 1-10 (16 mg, 19.84 μmol, 1 eq, HCl salt) and NMM (8.03 mg, 79.36 μmol, 8.73 μL, 4 eq) were added and the mixture was stirred at 25° C. for 2 hr. The mixture was purified directly by reversed-phase HPLC (0.1% NH₃·H₂O) without further work-up. I-656 (8.45 mg, 8.02 mol, 40.40% yield, 98.97% purity) was obtained as a white solid. LCMS (Method G): Rt=0.702 min, [M+H]⁺=1043.6. SFC: Rt=3.244 min, 4.302 min. ¹H NMR (400 MHz, METHANOL-d₄) δ=8.37 (d, J=7.9 Hz, 1H), 8.00-7.92 (m, 1H), 7.91-7.78 (m, 2H), 7.62-7.46 (m, 3H), 7.41-7.34 (m, 1H), 7.30-7.21 (m, 1H), 7.16 (t, J=9.1 Hz, 1H), 4.61-4.54 (m, 1H), 4.47-4.36 (m, 3H), 4.35-4.28 (m, 1H), 4.03-3.93 (m, 2H), 3.81-3.63 (m, 4H), 3.54-3.47 (m, 2H), 3.26-3.11 (m, 2H), 3.05-2.97 (m, 2H), 2.86-2.71 (m, 1H), 2.42-2.25 (m, 4H), 2.24-2.07 (m, 4H), 2.06-1.87 (m, 8H), 1.87-1.55 (m, 12H), 1.41-1.04 (m, 8H), 0.92-0.77 (m, 4H). ¹⁹F NMR (377 MHz, METHANOL-d₄) δ=−120.75 (br d, J=18.0 Hz, 1F), −121.68-−121.93 (m, 1F).

### Step 8: Synthesis of Intermediate 1-5B

To a solution of Intermediate 1-5A (22 g, 70.94 mmol, 1 eq) in THF (60 mL), MeOH (60 mL) and H₂O (60 mL) was added LiOH·H₂O (4.47 g, 106.41 mmol, 1.5 eq). The mixture was stirred at 25° C. for 1 hr. The reaction was concentrated under reduced pressure to give a residue. Then the aqueous phase was adjust to pH 5 with saturated aqueous citric acid, and extracted with EA (4 mL*3). Then the organic layers were dried over Na₂SO₄, filtered and concentrated under reduced pressure to give a residue. The crude product was used directly without further purification. Intermediate 1-5B (19.5 g, 65.86 mmol, 92.84% yield, 100% purity) was obtained as a white solid. ¹H NMR (400 MHz, CHLOROFORM-d) δ=12.95-9.25 (m, 1H), 4.02 (s, 2H), 3.17 (s, 2H), 2.12-1.92 (m, 6H), 1.85-1.70 (m, 2H).

### Step 9: Synthesis of Intermediate 1-5C

Solution 1: Intermediate 1-5B (19.5 g, 65.86 mmol, 1 eq) and TEA (10.00 g, 98.78 mmol, 13.75 mL, 1.5 eq) in t-BuOH (87.75 mL). Solution 2: DPPA (23.80 g, 86.47 mmol, 18.66 mL, 1.31 eq) in t-BuOH (87.75 mL). Solution 1 was pumped by Pump 1 (1.5 mL/min) to flow reactor 1 (SS,Coils reactor, 3.175 (⅛″) mm, 60 mL, 180° C.). Solution 2 was pumped by Pump 2 (1.5 mL/min) to flow reactor 1. The residence time of flow reactor 1 was 20 min. The pressure was set to 3.5 MPa. Pump 1 and Pump 2 were started at the same time. The reaction mixture was collected after running 20 min. Collection of the reaction mixture ended after 120 minutes. The mixture was concentrated in vacuum. The residue was purified by flash silica gel chromatography (ISCO; 330 g SepaFlash Silica Flash Column, Eluent of 0˜20% Ethyl acetate/Petroleum ether gradient @100 mL/min). Intermediate 1-5C (10 g, 27.23 mmol, 41.35% yield) was obtained as white solid. ¹H NMR (400 MHz, CHLOROFORM-d) δ=4.28 (br s, 1H), 3.96 (s, 2H), 3.16 (s, 2H), 2.18-1.99 (m, 4H), 1.93-1.77 (m, 4H), 1.43 (s, 9H).

### Step 10: Synthesis of Intermediate 1-5D

To a solution of Intermediate 1-5C (8.5 g, 23.15 mmol, 1 eq) in DMSO (80 mL) was added KOAc (5.68 g, 57.87 mmol, 2.5 eq), and the mixture was stirred at 100° C. for 16 hr. The mixture was poured into 200 mL of water and extracted with EA (100 mL*2). The combined organic phase was dried over anhydrous Na₂SO₄, filtered and concentrated in vacuum. The residue was purified by flash silica gel chromatography (ISCO; 330 g SepaFlash Silica Flash Column, Eluent of 0˜30% Ethyl acetate/Petroleum ether gradient @100 mL/min). Intermediate 1-5D (2.5 g, 8.35 mmol, 36.08% yield) was obtained as a white solid. Intermediate 1-5C (3.7 g, 10.08 mmol, 43.53% yield) was obtained as a white solid. ¹H NMR (400 MHz, CHLOROFORM-d) δ=4.30 (br s, 1H), 3.96 (s, 2H), 3.93 (s, 2H), 2.15-2.05 (m, 5H), 2.03-1.91 (m, 2H), 1.91-1.79 (m, 2H), 1.65 (dt, J=4.1, 11.9 Hz, 2H), 1.42 (s, 9H). ¹H NMR (400 MHz, CHLOROFORM-d) δ=4.28 (br s, 1H), 3.97 (s, 2H), 3.16 (s, 2H), 2.16-1.99 (m, 4H), 1.95-1.75 (m, 4H), 1.43 (s, 9H).

### Step 11: Synthesis of Intermediate 1-5E

To a solution of Intermediate 1-5D (2.5 g, 8.35 mmol, 1 eq) in MeOH (25 mL) was added NaOMe (5.4 M, 1.70 mL, 1.1 eq) dropwise at 0° C. under N₂. The mixture was stirred at 25° C. for 16 hr. The mixture was poured into water (100 mL) and extracted with EA (50 mL*2). The combined organic phase was dried over anhydrous Na₂SO₄, filtered and concentrated under vacuum. The residue was used directly without further purification. Intermediate 1-5E (1.7 g, 6.61 mmol, 79.11% yield) was obtained as a white solid. ¹H NMR (400 MHz, CHLOROFORM-d) δ=4.31 (br s, 1H), 3.97 (s, 2H), 3.39 (s, 2H), 2.17-2.06 (m, 2H), 2.05-1.97 (m, 2H), 1.87-1.80 (m, 2H), 1.59 (dt, J=3.9, 12.1 Hz, 2H).

### Step 12: Synthesis of Intermediate 1-5

To a solution of oxalyl dichloride (2.10 g, 16.52 mmol, 1.45 mL, 2.5 eq) in DCM (20 mL) was added DMSO (1.29 g, 16.52 mmol, 1.29 mL, 2.5 eq) in DCM (10 mL) dropwise at −70° C. under N₂. The mixture was stirred at −70° C. for 30 min. Then Intermediate 1-5E (1.7 g, 6.61 mmol, 1 eq) in DCM (10 mL) was added and the mixture was stirred at −70° C. for 2 hr. TEA (4.68 g, 46.25 mmol, 6.44 mL, 7 eq) was added and the mixture was warmed to 25° C. and the mixture was stirred for 1 hr. The mixture was poured into water (100 mL) and extracted with DCM (100 mL*2), the combined organic phase was dried over anhydrous Na₂SO₄, filtered and concentrated under vacuum. The residue was purified by flash silica gel chromatography (ISCO; 4 g SepaFlash Silica Flash Column, Eluent of 0˜50% Ethyl acetate/Petroleum ether gradient @10 mL/min). Intermediate 1-5 (1.4 g, 5.48 mmol, 83.00% yield) was obtained as a yellow solid. ¹H NMR (400 MHz, CHLOROFORM-d) δ=9.58 (s, 1H), 4.33 (br s, 1H), 4.07 (s, 2H), 2.20-2.11 (m, 2H), 2.01 (br dd, J=8.0, 10.5 Hz, 2H), 1.92-1.82 (m, 4H), 1.43 (s, 9H).

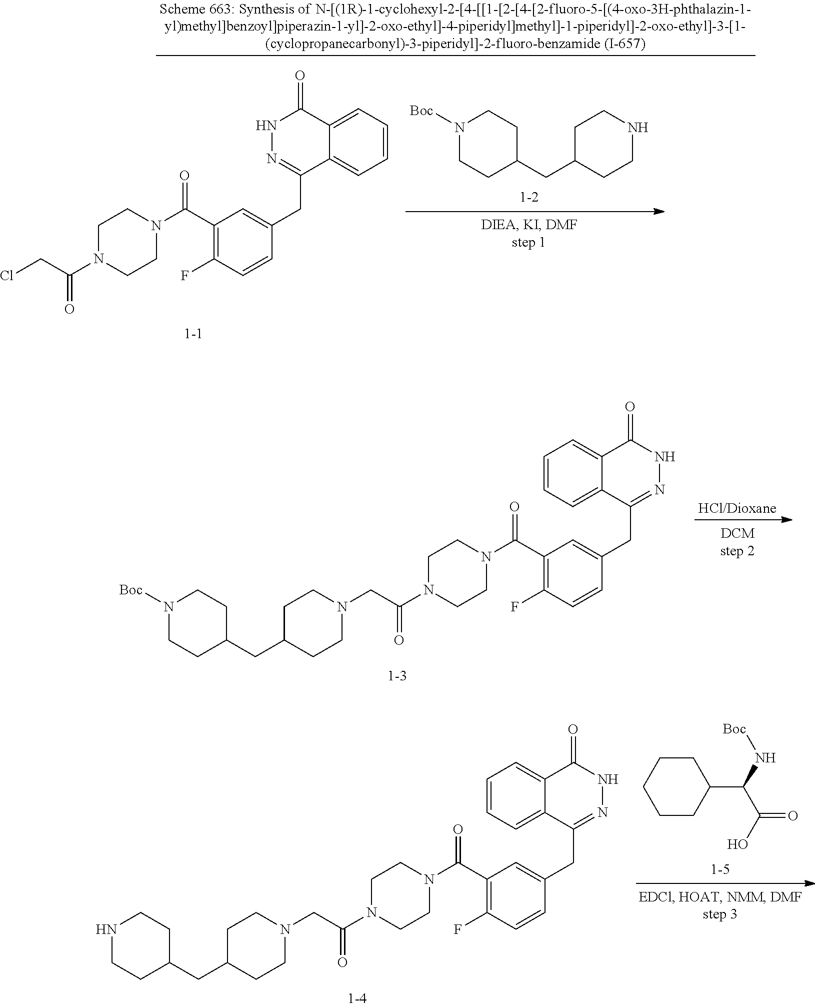

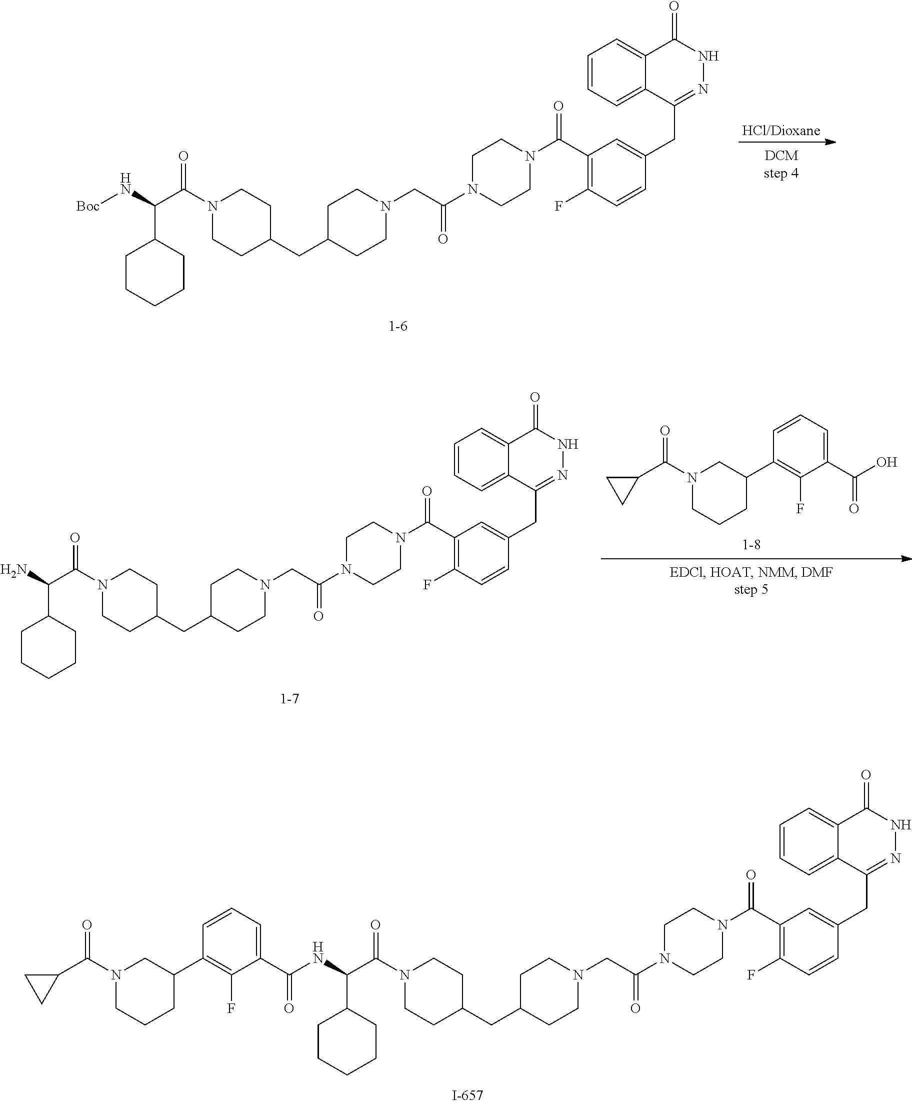

### Step 1: Synthesis of Intermediate 1-3

To a solution of Intermediate 1-1 (200 mg, 451.60 μmol, 1 eq) in DMF (2 mL) was added Intermediate 1-2 (127.54 mg, 451.60 μmol, 1 eq), DIEA (233.46 mg, 1.81 mmol, 314.64 μL, 4 eq) and KI (299.87 mg, 1.81 mmol, 4 eq). The mixture was stirred at 25° C. for 1 hr. The reaction mixture was triturated with water (8 ml), filtered and the filter cake was washed with water (3 mL*2). The filter cake was dried under vacuum. Intermediate 1-3 (300 mg, 435.52 μmol, 96.44% yield) was obtained as a white solid. LCMS (Method G): Rt=0.674 min, [M+H]⁺=689.5.

### Step 2: Synthesis of Intermediate 1-4

To a solution of Intermediate 1-3 (300 mg, 435.52 μmol, 1 eq) in DCM (1 mL) was added HCl/dioxane (2 M, 2 mL, 9.18 eq). The mixture was stirred at 25° C. for 1 hr. The mixture was concentrated under vacuum. The residue was used in the next step without purification. Intermediate 1-4 (270 mg, 431.88 μmol, 99.16% yield, HCl salt) was obtained as a white solid. LCMS (Method E): Rt=0.354 min, [M+H]⁺=589.2.

### Step 3: Synthesis of Intermediate 1-6

To a solution of Intermediate 1-4 (270 mg, 431.88 μmol, 1 eq, HCl salt) in DMF (3 mL) was added Intermediate 1-5 (111.13 mg, 431.88 μmol, 1 eq), EDCI (165.58 mg, 863.76 μmol, 2 eq), NMM (218.42 mg, 2.16 mmol, 237.41 μL, 5 eq) and HOAt (58.78 mg, 431.88 μmol, 60.41 μL, 1 eq). The mixture was stirred at 25° C. for 2 hr. The reaction mixture was triturated with water (9 ml), filtered and the filter cake was washed with water (3 mL*2). The filter cake was dried under vacuum. Intermediate 1-6 (300 mg, 362.31 μmol, 83.89% yield) was obtained as a white solid. LCMS (Method G): Rt=0.727 min, [M+H]⁺=828.6. SFC: Rt=1.595 min.

### Step 4: Synthesis of Intermediate 1-7

To a solution of Intermediate 1-6 (100 mg, 120.77 μmol, 1 eq) in DCM (0.5 mL) was added HCl/dioxane (4 M, 0.5 mL, 16.56 eq). The mixture was stirred at 25° C. for 0.5 hr. The mixture was concentrated under vacuum. Intermediate 1-7 (80 mg, crude, HCl salt) was obtained as a yellow oil was obtained. LCMS (Method E): Rt=0.408 min, [M+H]⁺=728.3.

### Step 5: Synthesis of I-657

To a solution of Intermediate 1-7 (40 mg, 52.33 μmol, 1 eq, HCl salt) in DMF (0.5 mL) was added Intermediate 1-8 (15.24 mg, 52.33 μmol, 1 eq), EDCI (20.06 mg, 104.66 μmol, 2 eq), NMM (26.47 mg, 261.65 μmol, 28.77 μL, 5 eq) and HOAt (7.12 mg, 52.33 μmol, 7.32 μL, 1 eq). The mixture was stirred at 25° C. for 1 hr. The mixture was diluted with water (2 mL) and extracted with EtOAc (5 mL*3). The organic layer was concentrated under vacuum. The residue was purified by Prep-HPLC (column: Waters Xbridge 150*25 mm*5 um; mobile phase: [water (NH₄HCO₃)-ACN]; gradient: 38%-68% B over 15 min) and dried by lyophilization. I-657 (30 mg, 28.68 μmol, 54.81% yield, 95.72% purity) was obtained as a white solid. LCMS (Method G): Rt=0.718 min, [M+H]⁺=1001.7. SFC: Rt=2.247 min, 2.967 min. ¹H NMR (400 MHz, METHANOL-d₄) δ=8.37 (d, J=7.5 Hz, 1H), 7.94 (br s, 1H), 7.89-7.83 (m, 2H), 7.59-7.46 (m, 3H), 7.40-7.36 (m, 1H), 7.26 (br d, J=6.8 Hz, 1H), 7.19-7.13 (m, 1H), 4.96 (br s, 1H), 4.60-4.49 (m, 2H), 4.39 (s, 3H), 4.27-4.18 (m, 1H), 3.81-3.66 (m, 4H), 3.57-3.48 (m, 2H), 3.27 (br d, J=1.2 Hz, 2H), 3.22-3.10 (m, 2H), 3.09-2.86 (m, 3H), 2.86-2.66 (m, 2H), 2.22-2.09 (m, 2H), 2.09-1.99 (m, 2H), 1.89-1.65 (m, 14H), 1.43 (br s, 1H), 1.38-0.95 (m, 13H), 0.91-0.77 (m, 4H). ¹⁹F NMR (377 MHz, METHANOL-d₄) δ=−76.91 (br s, 1F), −120.78 (br s, 1F), −121.61 (br s, 1F).

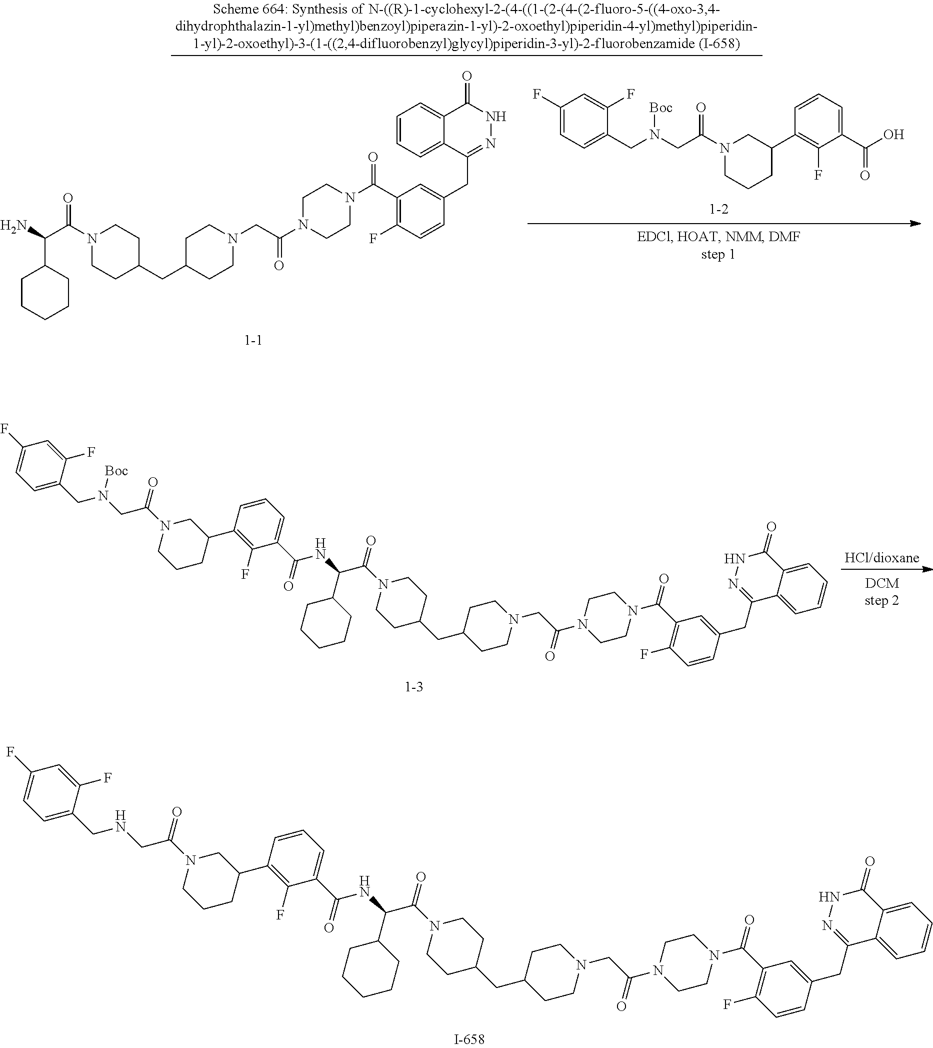 [SMILES: N[C@@H](C(=O)N1CCC(CC2CCN(CC(=O)N3CCN(C(=O)c4cc(Cc5n[nH]c(=O)c6ccccc56)ccc4F)CC3)CC2)CC1)C1CCCCC1 ; CC(C)(C)OC(=O)N(CC(=O)N1CCCC(c2cccc(C(=O)O)c2F)C1)Cc1ccc(F)cc1F ; CC(C)(C)OC(=O)N(CC(=O)N1CCCC(c2cccc(C(=O)N[C@@H](C(=O)N3CCC(CC4CCN(CC(=O)N5CCN(C(=O)c6cc(Cc7n[nH]c(=O)c8ccccc78)ccc6F)CC5)CC4)CC3)C3CCCCC3)c2F)C1)Cc1ccc(F)cc1F ; "HCl/dioxane" ; O=C(N[C@@H](C(=O)N1CCC(CC2CCN(CC(=O)N3CCN(C(=O)c4cc(Cc5n[nH]c(=O)c6ccccc56)ccc4F)CC3)CC2)CC1)C1CCCCC1)c1cccc(C2CCCN(C(=O)CNCc3ccc(F)cc3F)C2)c1F]

### Step 1: Synthesis of Intermediate 1-3

To a solution of Intermediate 1-1 (40.00 mg, 52.33 μmol, 1 eq, HCl salt) in DMF (0.5 mL) was added Intermediate 1-2 (26.51 mg, 52.33 μmol, 1 eq), EDCI (20.06 mg, 104.66 μmol, 2 eq), NMM (26.47 mg, 261.65 μmol, 28.77 μL, 5 eq) and HOAt (7.12 mg, 52.33 μmol, 7.32 μL, 1 eq). The mixture was stirred at 25° C. for 1 hr. The mixture was diluted with water (2 mL), extracted with EtOAc (5 mL*3) and washed with brine (5 mL). The organic layer was dried with anhydrous Na₂SO₄, filtered and concentrated under vacuum. The residue was used in the next step without purification. Intermediate 1-3 (50 mg, crude) as a white solid was obtained. LCMS (Method G): Rt=0.813 min, [M+H]⁺=1216.8.

### Step 2: Synthesis of I-658

To a solution of Intermediate 1-3 (50 mg, 41.10 μmol, 1 eq) in DCM (0.25 mL) was added HCl/dioxane (2 M, 0.5 mL, 24.33 eq). The mixture was stirred at 25° C. for 1 hr. The mixture was concentrated under vacuum. The residue was purified by Prep-HPLC (column: Waters Xbridge 150*25 mm*5 um; mobile phase: [water (NH₄HCO₃)-ACN]; gradient: 42%-72% B over 9 min) and dried by lyophilization. I-658 (20 mg, 17.92 μmol, 43.59% yield, 100% purity) as a white solid was obtained. LCMS (Method G): Rt=0.755 min, [M+H]⁺=1116.8. SFC: Rt=2.736 min, 3.407 min. ¹H NMR (400 MHz, METHANOL-d₄) δ=8.37 (d, J=7.6 Hz, 1H), 7.98-7.92 (m, 1H), 7.90-7.81 (m, 2H), 7.57 (br d, J=6.8 Hz, 1H), 7.54-7.42 (m, 3H), 7.42-7.34 (m, 1H), 7.29-7.21 (m, 1H), 7.19-7.15 (m, 1H), 6.96-6.92 (m, 2H), 5.01-4.95 (m, 1H), 4.61-4.47 (m, 2H), 4.39 (s, 2H), 4.23 (br d, J=9.2 Hz, 1H), 3.92-3.85 (m, 1H), 3.85-3.77 (m, 3H), 3.73 (br s, 2H), 3.66 (br s, 1H), 3.59-3.46 (m, 4H), 3.35-3.32 (m, 1H), 3.28 (br s, 1H), 3.24-2.98 (m, 4H), 2.97-2.84 (m, 2H), 2.84-2.63 (m, 2H), 2.15-1.96 (m, 3H), 1.92-1.57 (m, 14H), 1.47-0.93 (m, 13H). ¹⁹F NMR (377 MHz, METHANOL-d₄) δ=−76.93 (br s, 1F), −113.06-−113.49 (m, 1F), −116.20 (td, J=7.6, 29.7 Hz, 1F), −120.75 (br d, J=29.2 Hz, 1F), −121.57-−121.82 (m, 1F).

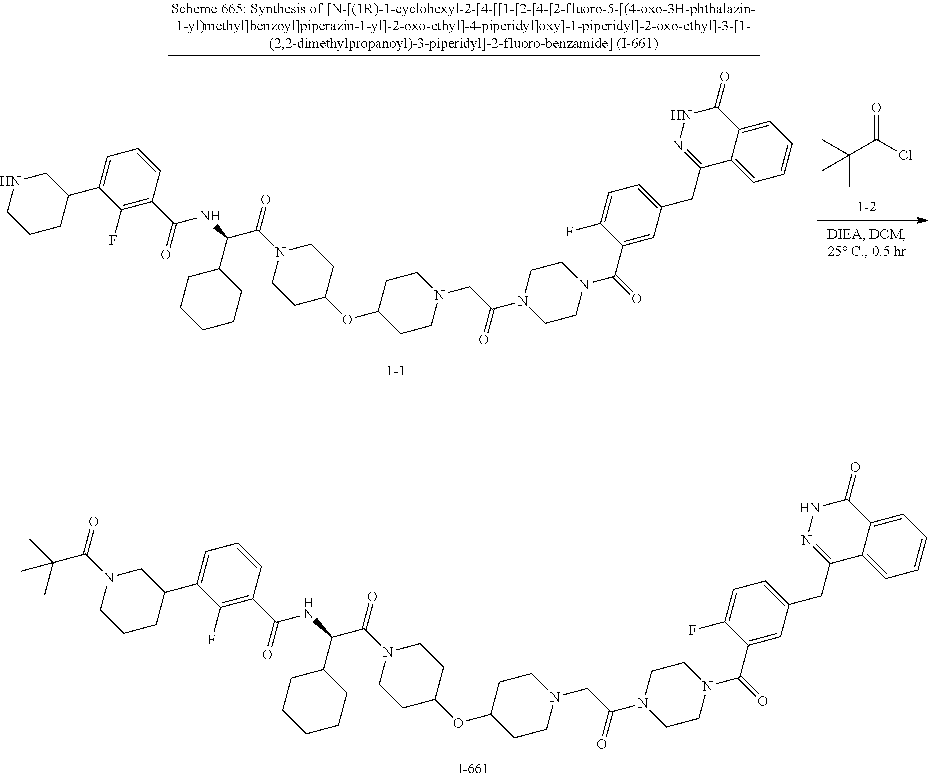

To a mixture of intermediate 1-1 (100 mg, 102.93 μmol, 1 eq, HCl salt) in DCM (1 mL) was added intermediate 1-2 (14.89 mg, 123.51 μmol, 15.20 μL, 1.2 eq) and DIEA (26.60 mg, 205.85 μmol, 35.86 μL, 2 eq) at 25° C. The mixture was stirred at 25° C. for 0.5 h. The reaction mixture was concentrated to give a residue. The residue was purified by prep-HPLC (column: CD01-Phenomenex luna C18 150*25*10 um; mobile phase: [water (FA)-ACN]; gradient: 24%-54% B over 8 min) and lyophilizated to give the product. I-661 (44 mg, 41.30 μmol, 40.13% yield, 100% purity, FA salt) was obtained as a white solid. LCMS (Method D): Retention time: 0.407 min, [M+H]⁺=1019.6. SFC: Retention time: 3.330 min, 4.766 min. ¹H NMR (400 MHz, METHANOL-d4) δ=8.49 (s, 1H), 8.37 (d, J=7.6 Hz, 1H), 7.95 (d, J=7.6 Hz, 1H), 7.91-7.81 (m, 2H), 7.59-7.45 (m, 3H), 7.41-7.34 (m, 1H), 7.26 (t, J=7.6 Hz, 1H), 7.17 (t, J=8.8 Hz, 1H), 4.96 (d, J=7.6 Hz, 1H), 4.49 (d, J=12.0 Hz, 2H), 4.39 (s, 2H), 4.08-3.99 (m, 1H), 3.96-3.83 (m, 1H), 3.78 (d, J=19.2 Hz, 4H), 3.70-3.57 (m, 4H), 3.55-3.46 (m, 4H), 3.28-3.21 (m, 1H), 3.16-3.01 (m, 2H), 2.94 (s, 4H), 2.65-2.37 (m, 2H), 2.09-1.92 (m, 4H), 1.92-1.82 (m, 6H), 1.79 (d, J=2.4 Hz, 2H), 1.73-1.60 (m, 6H), 1.56-1.44 (m, 1H), 1.30 (s, 9H), 1.27-1.17 (m, 2H), 1.14 (t, J=11.2 Hz, 2H). ¹⁹F NMR (376 MHz, METHANOL-d4) δ=−120.785 (s, 1F), −121.610 (s, 1F).

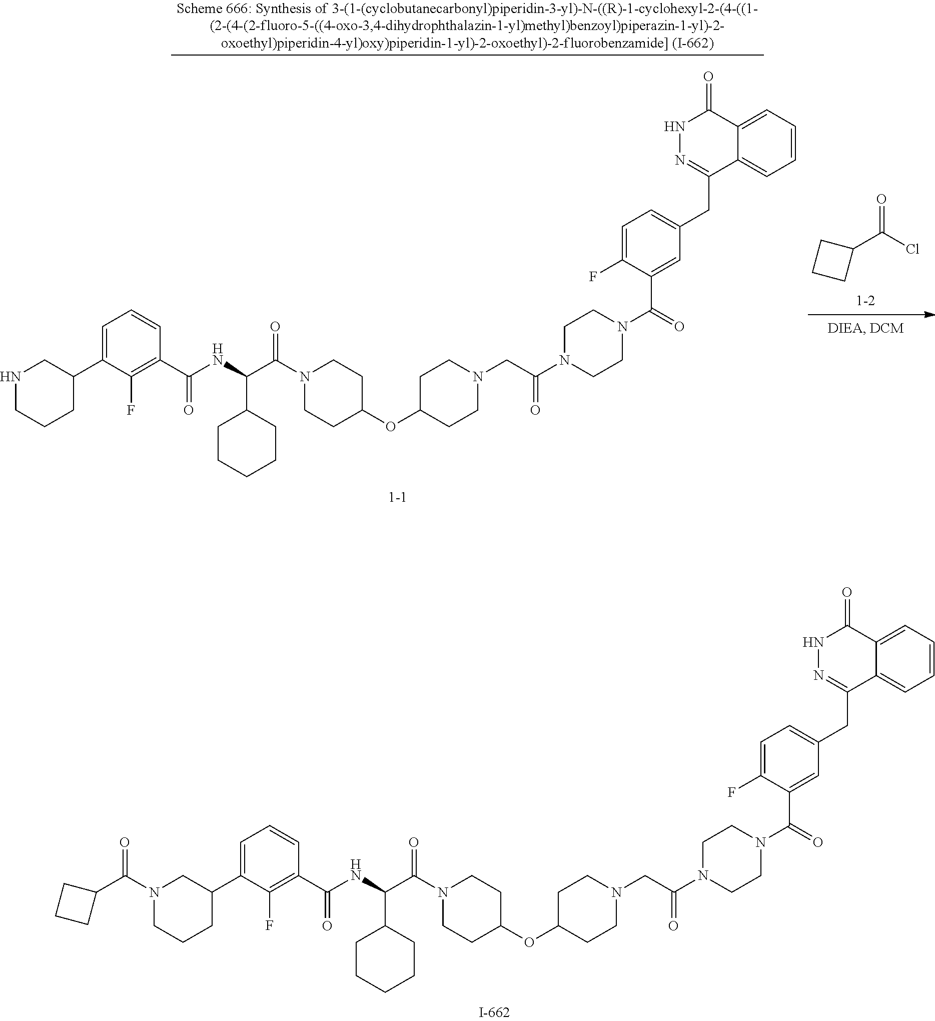 [SMILES: O=C(N[C@@H](C(=O)N1CCC(OC2CCN(CC(=O)N3CCN(C(=O)c4cc(Cc5n[nH]c(=O)c6ccccc56)ccc4F)CC3)CC2)CC1)C1CCCCC1)c1cccc(C2CCCNC2)c1F ; O=C(Cl)C1CCC1 ; O=C(N[C@@H](C(=O)N1CCC(OC2CCN(CC(=O)N3CCN(C(=O)c4cc(Cc5n[nH]c(=O)c6ccccc56)ccc4F)CC3)CC2)CC1)C1CCCCC1)c1cccc(C2CCCN(C(=O)C3CCC3)C2)c1F]

To a solution of Intermediate 1-1 (100 mg, 102.93 μmol, 1 eq, HCl salt) in DCM (1 mL) was added DIEA (26.60 mg, 205.85 μmol, 35.86 μL, 2 eq) and Intermediate 1-2 (14.64 mg, 123.51 μmol, 14.09 L, 1.2 eq). The reaction mixture was stirred at 25° C. for 1 hr. To the reaction mixture was added H₂O (2 mL) and then it was extracted with DCM (2 mL*3), the combined organic phase was washed, dried by Na₂SO₄, filtered and concentrated. The crude product was purified by reversed phase chromatography (0.1% FA condition), and the eluent was concentrated to remove MeCN and lyophilized to give product. I-662 (30.12 mg, 27.84 μmol, 27.05% yield, 98.288% purity, FA salt) was obtained as a white solid. LCMS (Method D): Rt=0.406 min, [M+H]⁺=1017.6. SFC: Rt=4.503 min, 6.642 min. ¹H NMR (400 MHz, METHANOL-d4) δ=8.44 (s, 1H), 8.37 (d, J=7.6 Hz, 1H), 7.94 (d, J=6.8 Hz, 1H), 7.91-7.79 (m, 2H), 7.61-7.43 (m, 3H), 7.37 (t, J=6.4 Hz, 1H), 7.30-7.21 (m, 1H), 7.17 (t, J=8.8 Hz, 1H), 4.99-4.92 (m, 1H), 4.57 (d, J=7.2 Hz, 1H), 4.39 (s, 2H), 4.09-3.97 (m, 1H), 3.94-3.87 (m, 1H), 3.86-3.73 (m, 4H), 3.73-3.57 (m, 5H), 3.55-3.40 (m, 4H), 3.38-3.33 (m, 1H), 3.28-3.23 (m, 1H), 3.15-2.98 (m, 4H), 2.84-2.58 (m, 3H), 2.36-2.08 (m, 4H), 2.05-1.91 (m, 5H), 1.90-1.74 (m, 9H), 1.69 (d, J=11.6 Hz, 3H), 1.64-1.43 (m, 3H), 1.41-1.23 (m, 3H), 1.23-1.00 (m, 3H). ¹⁹F NMR (377 MHz, METHANOL-d₄), δ=−120.764, −121.680.

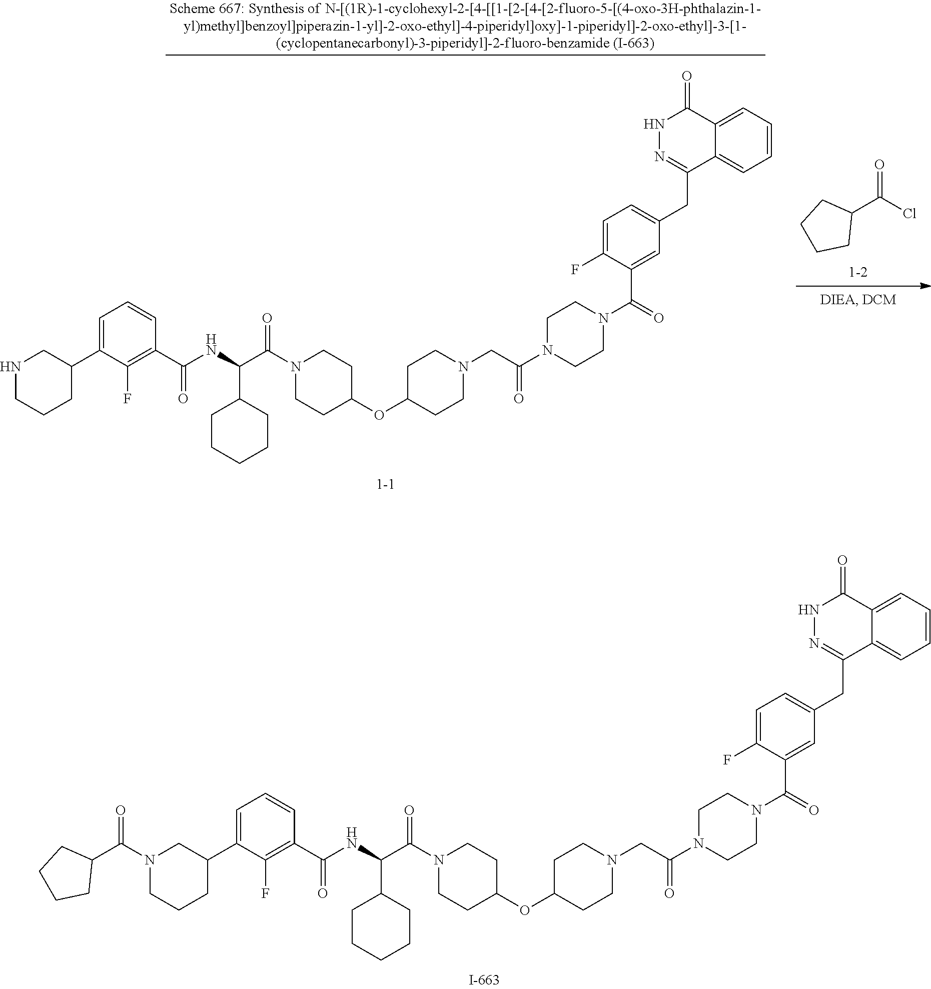 [SMILES: O=C(N[C@@H](C(=O)N1CCC(OC2CCN(CC(=O)N3CCN(C(=O)c4cc(Cc5n[nH]c(=O)c6ccccc56)ccc4F)CC3)CC2)CC1)C1CCCCC1)c1cccc(C2CCCNC2)c1F ; O=C(Cl)C1CCCC1 ; O=C(N[C@@H](C(=O)N1CCC(OC2CCN(CC(=O)N3CCN(C(=O)c4cc(Cc5n[nH]c(=O)c6ccccc56)ccc4F)CC3)CC2)CC1)C1CCCCC1)c1cccc(C2CCCN(C(=O)C3CCCC3)C2)c1F]

To a solution of intermediate 1-1 (80 mg, 82.34 μmol, 1 eq, HCl salt) and intermediate 1-2 (10.92 mg, 82.34 μmol, 10.01 μL, 1 eq) in DCM (0.8 mL) was added DIEA (21.28 mg, 164.68 μmol, 28.68 μL, 2 eq), and the mixture was stirred at 25° C. for 1 hr. The reaction mixture was washed with water (1 mL) and extracted with DCM (1 mL*3), the combined organic phase was dried by Na₂SO₄, and concentrated under reduced pressure to give a residue. The crude was purified by prep-HPLC (column: CD01-Phenomenex luna C18 150*25*10 um; mobile phase: [water (FA)-ACN]; gradient: 23%-53% B over 10 min) and the eluent was concentrated to remove ACN and lyophilized to give product. I-663 (46 mg, 42.70 μmol, 51.86% yield, 100% purity, FA salt) was obtained as a white solid. LCMS (Method D): Rt=0.414 min, [M+H]⁺=1031.6. SFC: Rt=4.125 min, 5.938 min. ¹H NMR (400 MHz, METHANOL-d₄) δ=8.49 (s, 1H), 8.36 (d, J=7.6 Hz, 1H), 7.95 (d, J=7.2 Hz, 1H), 7.90-7.80 (m, 2H), 7.60-7.46 (m, 3H), 7.41-7.34 (m, 1H), 7.30-7.21 (m, 1H), 7.16 (t, J=8.8 Hz, 1H), 5.00-4.93 (m, 1H), 4.61 (d, J=8.8 Hz, 1H), 4.38 (s, 2H), 4.22-4.11 (m, 1H), 4.07-3.99 (m, 1H), 3.97-3.83 (m, 1H), 3.77 (d, J=18.8 Hz, 3H), 3.71-3.55 (m, 5H), 3.54-3.43 (m, 4H), 3.37-3.33 (m, 1H), 3.26-2.89 (m, 6H), 2.84-2.50 (m, 3H), 2.05-1.82 (m, 11H), 1.80-1.59 (m, 14H), 1.52-1.41 (m, 1H), 1.36-1.21 (m, 3H), 1.20-1.09 (m, 2H). ¹⁹F NMR (377 MHz, METHANOL-d₄), δ=−120.747, −121.708.

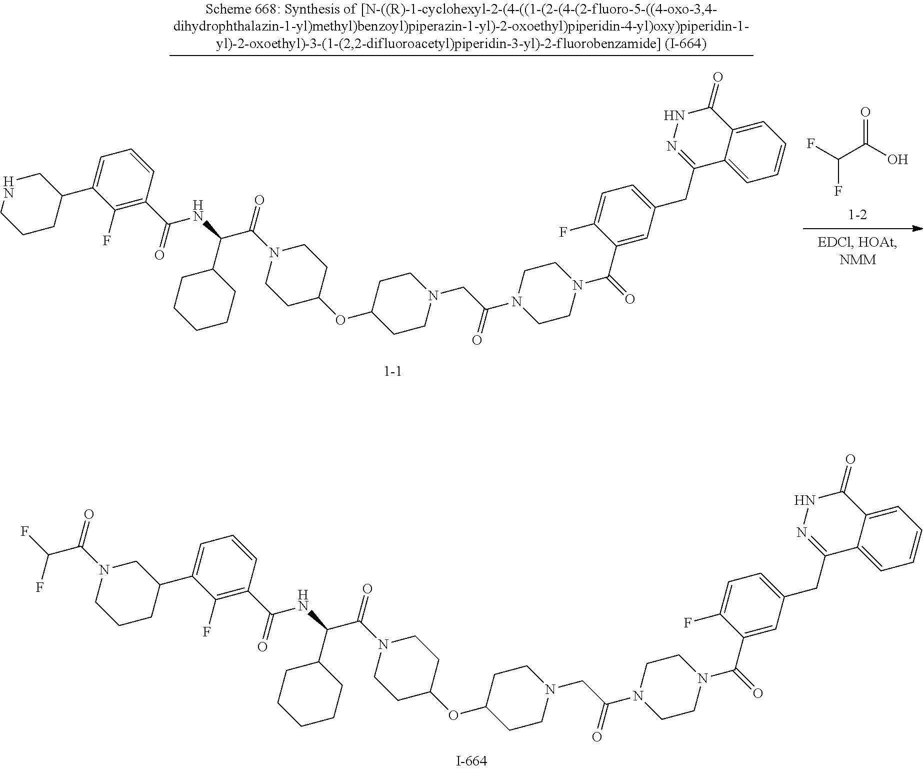

To a solution of Intermediate 1-1 (0.1 g, 102.93 μmol, 1 eq, HCl salt) and Intermediate 1-2 (14.83 mg, 154.39 μmol, 9.72 μL, 1.5 eq) in DMF (1 mL) was added EDCI (59.19 mg, 308.78 μmol, 3 eq), HOAt (14.01 mg, 102.93 μmol, 14.40 μL, 1 eq), and NMM (52.05 mg, 514.63 μmol, 56.58 μL, 5 eq). The mixture was stirred at 25° C. for 0.25 hr. The reaction mixture was filtered and concentrated under reduced pressure to give a residue. The residue was purified by reversed phase chromatography (0.1% FA) and the eluent was concentrated to remove MeCN and lyophilized to give I-664 (44.83 mg, 41.92 μmol, 40.73% yield, 99.05% purity, FA salt) as a white solid. LCMS (Method D): Rt=0.375 min, [M+H]⁺=1013.6. SFC: Rt=2.681 min, 3.264 min. ¹H NMR (400 MHz, METHANOL-d₄) δ=8.48 (s, 1H), 8.37 (d, J=7.6 Hz, 1H), 7.95 (d, J=7.6 Hz, 1H), 7.91-7.79 (m, 2H), 7.63-7.44 (m, 3H), 7.37 (s, 1H), 7.31-7.22 (m, 1H), 7.19-7.14 (m, 1H), 6.64-6.33 (m, 1H), 4.95 (d, J=6.8 Hz, 1H), 4.60-4.51 (m, 1H), 4.39 (s, 2H), 4.11-3.99 (m, 2H), 3.78 (d, J=18.0 Hz, 5H), 3.70-3.55 (m, 5H), 3.51 (s, 4H), 3.28-3.06 (m, 2H), 3.01-2.77 (m, 3H), 2.71-2.49 (m, 2H), 2.07-2.01 (m, 1H), 1.93 (d, J=9.6 Hz, 5H), 1.86 (d, J=10.4 Hz, 4H), 1.78 (s, 3H), 1.69 (d, J=10.4 Hz, 5H), 1.58-1.46 (m, 1H), 1.35-1.21 (m, 3H), 1.20-1.09 (m, 2H). ¹⁹F NMR (377 MHz, METHANOL-d₄) δ=−120.770, −121.550, −121.708, −125.250, −125.610, −125.790.

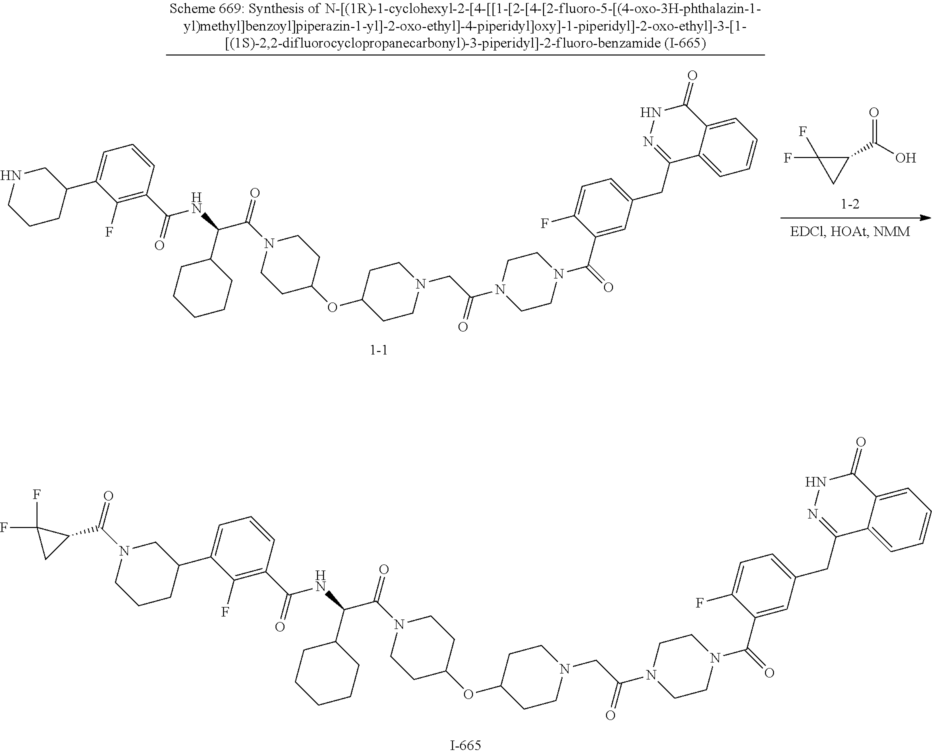

To a solution of intermediate 1-1 (80 mg, 82.34 μmol, 1 eq, HCl salt) in DMF (0.8 mL) was added NMM (41.64 mg, 411.70 μmol, 45.26 μL, 5 eq) at 25° C., and the mixture was stirred at 25° C. for 10 min. Then intermediate 1-2 (10.05 mg, 82.34 μmol, 1 eq), EDCI (78.92 mg, 411.70 μmol, 5 eq) and HOAt (22.42 mg, 164.68 μmol, 23.04 μL, 2 eq) were added at 25° C. The resulting mixture was stirred at 25° C. for 1 hr. The reaction mixture was washed with water (1 mL) and extracted with DCM (1 mL*3), the combined organic phase was dried by Na₂SO₄, and concentrated under reduced pressure to give a residue. The crude was purified by prep-HPLC (column: CD01-Phenomenex luna C18 150*25*10 um; mobile phase: [water(FA)-MeCN]; gradient: 20%-500% B over 10 min) and the eluent was concentrated to remove MeCN and lyophilized to give product. I-665 (52 mg, 47.63 μmol, 57.84% yield, 99.399% purity, FA salt) was obtained as a white solid. LCMS (Method D): Rt=0.416 min, 0.422 min, [M+H]⁺=1039.3. SFC: Rt=3.232 min, 3.790 min. ¹H NMR (400 MHz, METHANOL-d₄) δ=8.46 (s, 1H), 8.37 (d, J=7.6 Hz, 1H), 7.95 (d, J=7.2 Hz, 1H), 7.90-7.81 (m, 2H), 7.61-7.47 (m, 3H), 7.38 (t, J=6.0 Hz, 1H), 7.30-7.22 (m, 1H), 7.17 (t, J=8.8 Hz, 1H), 4.95 (t, J=7.2 Hz, 1H), 4.63-4.53 (m, 1H), 4.39 (s, 2H), 4.26-4.14 (m, 1H), 4.09-3.99 (m, 1H), 3.96-3.86 (m, 1H), 3.78 (d, J=18.0 Hz, 4H), 3.72-3.58 (m, 5H), 3.57-3.45 (m, 4H), 3.38-3.34 (m, 1H), 3.30-3.19 (m, 2H), 3.16-2.88 (m, 5H), 2.87-2.52 (m, 3H), 2.04-1.92 (m, 6H), 1.86 (d, J=8.8 Hz, 3H), 1.77 (d, J=8.8 Hz, 4H), 1.69 (d, J=10.8 Hz, 4H), 1.58-1.50 (m, 1H), 1.34-1.21 (m, 3H), 1.19-1.09 (m, 2H). ¹⁹F NMR (377 MHz, METHANOL-d₄) δ=−120.764, −121.642, −121.755, −128.442, −128.870, −140.489, −141.292, −141.697.

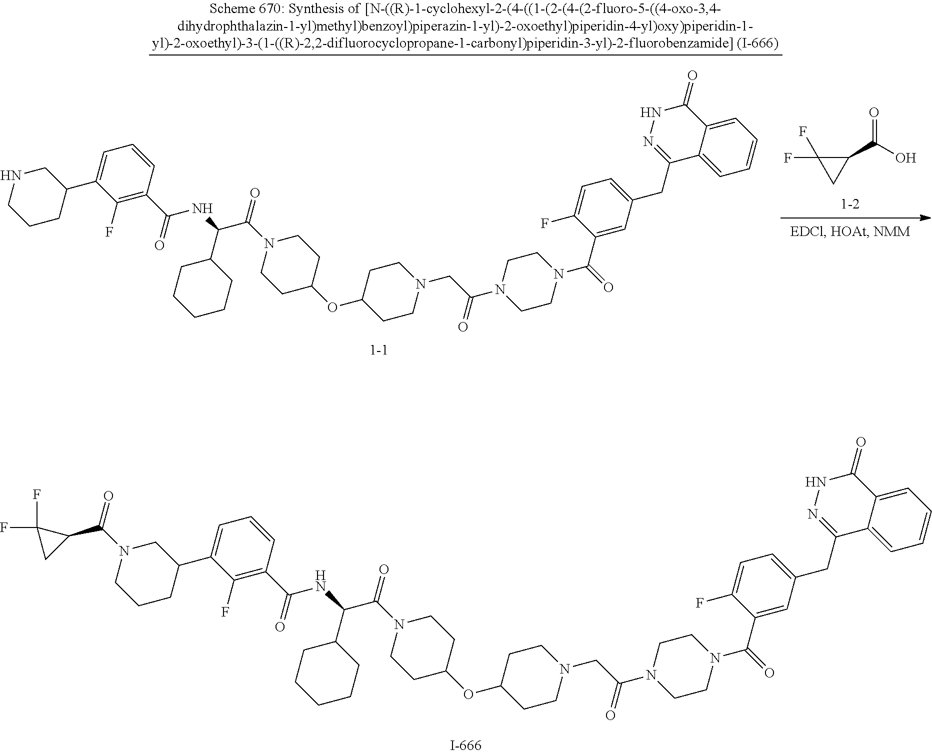

To a solution of Intermediate 1-1 (0.1 g, 102.93 μmol, 1 eq, HCl salt) and Intermediate 1-2 (18.85 mg, 154.39 μmol, 1.5 eq) in DMF (1 mL) was added EDCI (59.19 mg, 308.78 μmol, 3 eq), HOAt (14.01 mg, 102.93 μmol, 14.40 μL, 1 eq), and NMM (52.05 mg, 514.63 μmol, 56.58 μL, 5 eq). The mixture was stirred at 25° C. for 0.25 h. The reaction mixture was filtered and concentrated under reduced pressure to give a residue. The residue was purified by prep-HPLC (column: CD01-Phenomenex luna C18 150*25*10 um; mobile phase: [water (FA)-MeCN]; gradient: 20%-50% B over 10 min) and the eluent was concentrated to remove MeCN and lyophilized to give I-666 (49.13 mg, 45.27 μmol, 43.99% yield, 100% purity, FA salt) as a yellow solid. LCMS (Method D): Rt=0.391 min, [M+H]⁺=1039.6. SFC: Rt=2.809 min, 3.773 min. ¹H NMR (400 MHz, METHANOL-d4) δ=8.49 (d, J=6.8 Hz, 1H), 8.37 (d, J=7.6 Hz, 1H), 7.95 (d, J=7.6 Hz, 1H), 7.91-7.80 (m, 2H), 7.62-7.46 (m, 3H), 7.37 (s, 1H), 7.31-7.21 (m, 1H), 7.21-7.13 (m, 1H), 4.95 (d, J=7.6 Hz, 1H), 4.63-4.54 (m, 1H), 4.39 (s, 2H), 4.30-4.14 (m, 1H), 4.03 (d, J=9.2 Hz, 2H), 3.86-3.71 (m, 4H), 3.67 (s, 4H), 3.57-3.43 (m, 5H), 3.28-2.73 (m, 7H), 2.66-2.47 (m, 2H), 2.09-1.90 (m, 7H), 1.89-1.81 (m, 4H), 1.77 (d, J=7.6 Hz, 3H), 1.69 (d, J=10.4 Hz, 5H), 1.58-1.48 (m, 1H), 1.33-1.21 (m, 3H), 1.20-1.09 (m, 2H). ¹⁹F NMR (377 MHz, METHANOL-d₄) δ=−120.770, −121.550, −121.843, −128.425, −128.860, −140.485, −140.718, −140.920, −141.258, −141.663.

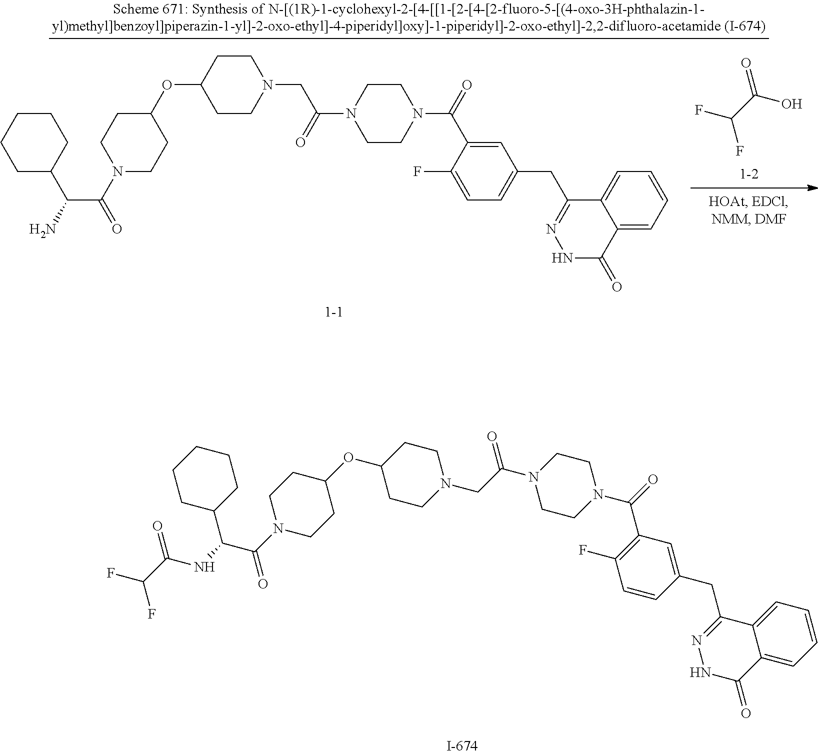 [SMILES: N[C@@H](C(=O)N1CCC(OC2CCN(CC(=O)N3CCN(C(=O)c4cc(Cc5n[nH]c(=O)c6ccccc56)ccc4F)CC3)CC2)CC1)C1CCCCC1 ; O=C(O)C(F)F ; O=C(N[C@@H](C(=O)N1CCC(OC2CCN(CC(=O)N3CCN(C(=O)c4cc(Cc5n[nH]c(=O)c6ccccc56)ccc4F)CC3)CC2)CC1)C1CCCCC1)C(F)F]

To a solution of Intermediate 1-1 (80 mg, 104.39 μmol, 1 eq, HCl salt) and Intermediate 1-2 (10.03 mg, 104.39 μmol, 6.57 μL, 1 eq) in DMF (1 mL) was added HOAt (14.21 mg, 104.39 μmol, 14.60 L, 1 eq), EDCI (60.04 mg, 313.18 μmol, 3 eq) and NMM (52.79 mg, 521.96 μmol, 57.39 μL, 5 eq). The mixture was stirred at 40° C. for 1 hour. The reaction mixture was diluted with water (60 mL) and extracted with EA (20 mL*2). The combined organic layers were concentrated under reduced pressure to give a residue. The crude product was purified by prep-HPLC (column: CD01-Phenomenex luna C18 150*25*10 um; mobile phase: [water (FA)-ACN]; gradient: 10%-40% B over 10 min). The eluent was concentrated under reduced pressure to remove ACN and then lyophilized to give the product. I-674 (26.14 mg, 30.46 μmol, 29.17% yield, 99.492% purity, FA salt) was obtained as a white solid. LCMS (Method D): Rt=0.365 min, [M+H]⁺=808.3. SFC: Rt=1.532 min. ¹H NMR (400 MHz, METHANOL-d₄) δ=8.41 (s, 1H), 8.37 (d, J=7.6 Hz, 1H), 7.99-7.92 (m, 1H), 7.91-7.79 (m, 2H), 7.50 (s, 1H), 7.41-7.32 (m, 1H), 7.21-7.12 (m, 1H), 6.25-5.92 (m, 1H), 4.76 (d, J=8.0 Hz, 1H), 4.39 (s, 2H), 4.04-3.93 (m, 2H), 3.87 (d, J=5.6 Hz, 1H), 3.78 (d, J=11.2 Hz, 5H), 3.70 (s, 1H), 3.61-3.50 (m, 3H), 3.43 (s, 1H), 3.36 (s, 1H), 3.24 (d, J=11.6 Hz, 3H), 3.00 (d, J=2.8 Hz, 2H), 2.03 (d, J=7.6 Hz, 2H), 1.94-1.73 (m, 8H), 1.71-1.60 (m, 3H), 1.56-1.46 (m, 1H), 1.30-1.17 (m, 3H), 1.11-0.99 (m, 2H). ¹⁹F NMR (377 MHz, METHANOL-d₄) δ=−120.667, −120.734, −127.722, −127.767.

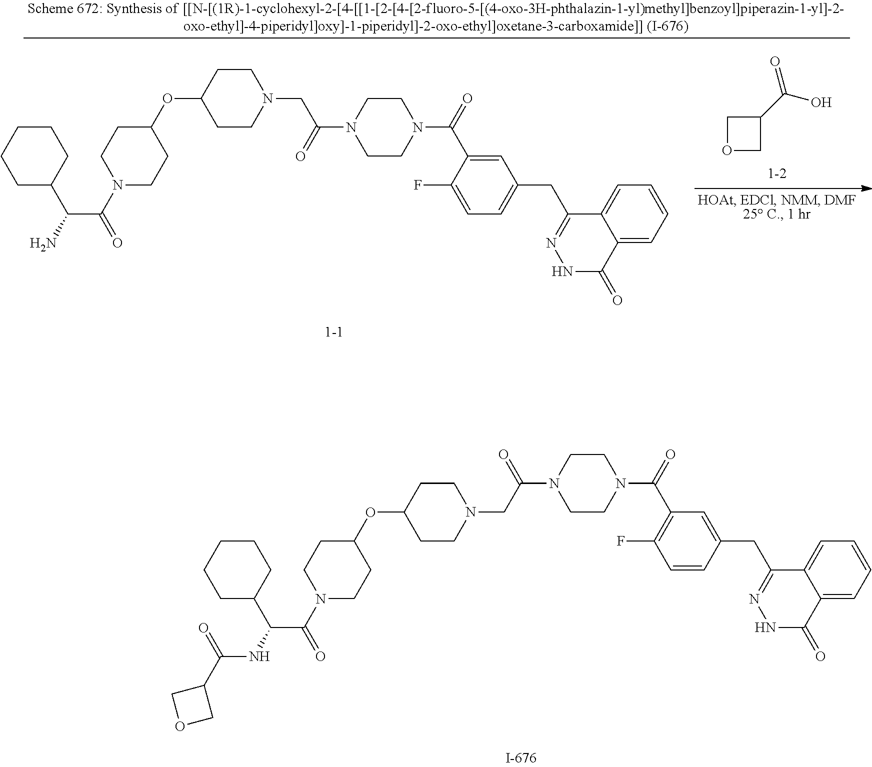 [SMILES: N[C@@H](C(=O)N1CCC(OC2CCN(CC(=O)N3CCN(C(=O)c4cc(Cc5n[nH]c(=O)c6ccccc56)ccc4F)CC3)CC2)CC1)C1CCCCC1 ; O=C(O)C1COC1 ; "HOAt, EDCl, NMM, DMF25° C., 1 hr" ; O=C(N[C@@H](C(=O)N1CCC(OC2CCN(CC(=O)N3CCN(C(=O)c4cc(Cc5n[nH]c(=O)c6ccccc56)ccc4F)CC3)CC2)CC1)C1CCCCC1)C1COC1]

To a solution of Intermediate 1-2 (13.32 mg, 130.49 μmol, 1 eq) in DMF (1 mL) was added EDCI (75.05 mg, 391.47 μmol, 3 eq), HOAt (17.76 mg, 130.49 μmol, 18.25 μL, 1 eq), Intermediate 1-1 (100 mg, 130.49 μmol, 1 eq, HCl salt) and NMM (65.99 mg, 652.45 μmol, 71.73 μL, 5 eq). The mixture was stirred at 25° C. for 1 hr. The mixture was filtered. The filtrate was purified by reversed-phase HPLC (0.1% FA condition). The eluent was concentrated under reduced pressure to remove ACN and then lyophilized to give the crude. The residue was purified by prep-HPLC (column: CD01-Phenomenex luna C18 150*25*10 um; mobile phase: [water(FA)-ACN]; gradient: 10%-40% B over 10 min). The eluent was concentrated under reduced pressure to remove ACN and then lyophilized to give I-676 (21.88 mg, 25.26 μmol, 19.36% yield, 99.3% purity, FA salt) as a white solid. LCMS (Method D): Rt=0.347 min, [M+H]⁺=814.4. SFC: Rt=1.649 min. ¹H NMR (400 MHz, METHANOL-d4) δ=8.42 (s, 1H), 8.36 (d, J=7.6 Hz, 1H), 7.98-7.92 (m, 1H), 7.91-7.80 (m, 2H), 7.50 (s, 1H), 7.41-7.34 (m, 1H), 7.21-7.13 (m, 1H), 4.84-4.69 (m, 5H), 4.38 (s, 2H), 4.07-3.68 (m, 10H), 3.59-3.53 (m, 2H), 3.50-3.32 (m, 4H), 3.29-3.21 (m, 2H), 3.07 (s, 2H), 2.10-1.45 (m, 15H), 1.32-1.13 (m, 3H), 1.11-0.97 (m, 2H). ¹⁹F NMR (376 MHz, METHANOL-d4) δ=−120.712.

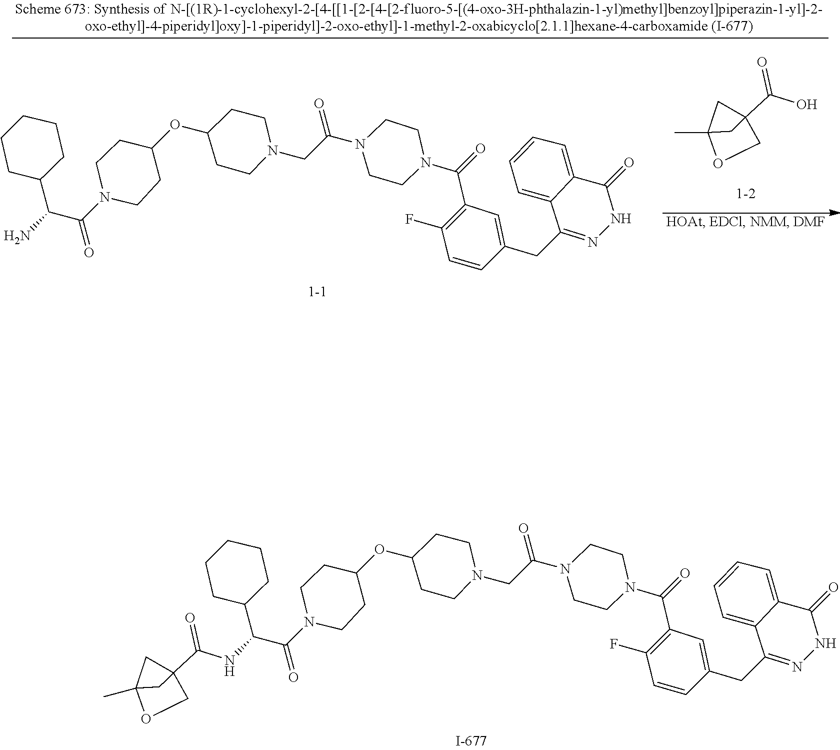

To a solution of Intermediate 1-2 (20 mg, 140.69 μmol, 1 eq) in DMF (0.5 mL) was added HOAt (19.15 mg, 140.69 μmol, 19.68 μL, 1 eq), EDCI (53.94 mg, 281.39 μmol, 2 eq), NMM (71.15 mg, 703.47 μmol, 77.34 μL, 5 eq) and Intermediate 1-1 (107.82 mg, 140.69 μmol, 1 eq, HCl salt). The mixture was stirred at 25° C. for 1 h. The reaction mixture was quenched with H₂O (1 mL) and extracted with EA (0.5 mL*3). The combined organic layers were washed with brine (0.5 mL*2), dried over anhydrous Na₂SO₄, filtered and concentrated under reduced pressure to give a residue. The residue was purified by Prep-HPLC (column: Waters Xbridge 150*25 mm*5 um; mobile phase: [water (ammonia hydroxide v/v)-ACN]; gradient: 22%-52% B over 12 min). I-677 (24.99 mg, 29.13 μmol, 20.70% yield, 99.535% purity) was obtained as a white solid. LCMS (Method G): Rt=0.576 min, [M+H]⁺=854.5. SFC: Rt=0.699 min. ¹H NMR (400 MHz, METHANOL-d₄) δ=8.36 (d, J=7.6 Hz, 1H), 7.94 (br d, J=7.6 Hz, 1H), 7.90-7.79 (m, 2H), 7.48 (br s, 1H), 7.41-7.35 (m, 1H), 7.15-7.13 (m, 1H), 4.76 (br d, J=8.4 Hz, 1H), 4.37 (s, 2H), 4.00-3.64 (m, 9H), 3.60-3.38 (m, 5H), 3.29-3.16 (m, 3H), 2.76 (br s, 2H), 2.25 (br d, J=9.6 Hz, 2H), 2.01-1.97 (m, 2H), 1.91-1.70 (m, 11H), 1.67-1.47 (m, 6H), 1.40 (s, 3H), 1.30-1.19 (m, 3H), 1.00 (br d, J=9.6 Hz, 2H). ¹⁹F NMR (377 MHz, METHANOL-d₄) δ=−120.666.

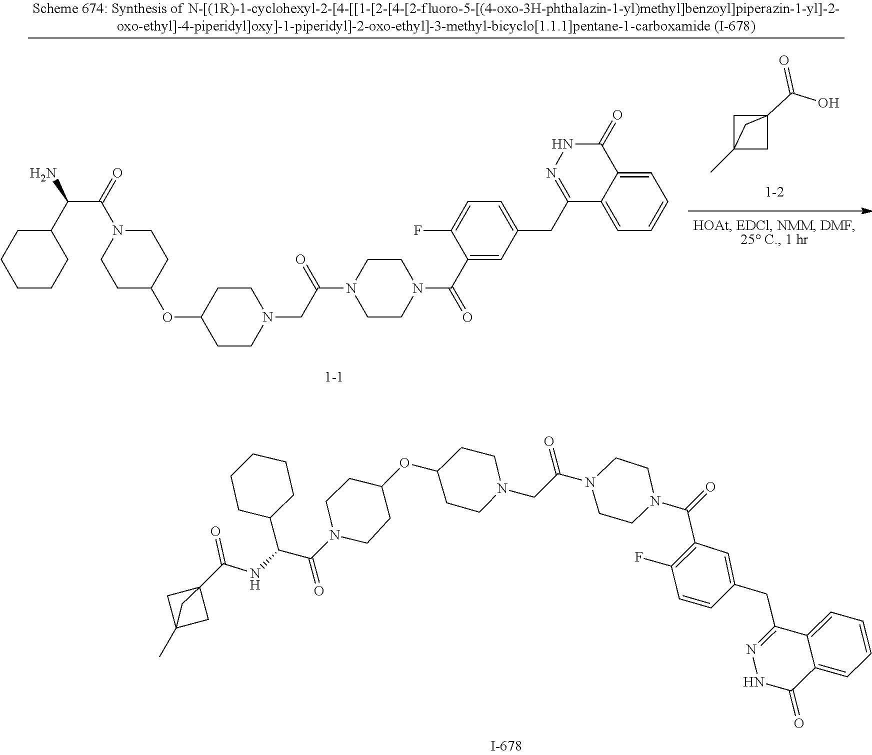

To a solution of Intermediate 1-2 (13.17 mg, 104.39 μmol, 1 eq) in DMF (1 mL) was added EDCI (60.04 mg, 313.18 μmol, 3 eq), HOAt (14.21 mg, 104.39 μmol, 14.60 μL, 1 eq) and NMM (52.79 mg, 521.96 μmol, 57.39 μL, 5 eq). Then Intermediate 1-1 (80 mg, 104.39 μmol, 1 eq, HCl salt) was added. The mixture was stirred at 25° C. for 1 hr. The mixture was washed with water (2 mL) and extracted with EA (3 mL*3). The combined organic layers were dried over sodium sulfate, filtered and concentrated under reduced pressure to give a residue. The residue was purified by prep-HPLC (column: CD01-Phenomenex luna C18 150*25*10 um; mobile phase: [water(FA)-ACN]; gradient: 18%-48% B over 10 min) and the eluent was concentrated under reduced pressure to remove ACN and lyophilized to give product. I-678 (37.49 mg, 41.35 μmol, 39.61% yield, 97.515% purity, FA salt) was obtained as a white solid. LCMS (Method D): Rt=0.385 min, [M+H]⁺=838.9. SFC: Rt=1.293 min. ¹H NMR (400 MHz, METHANOL-d4) δ=8.39 (s, 1H), 8.37 (d, J=7.6 Hz, 1H), 7.98-7.92 (m, 1H), 7.90-7.81 (m, 2H), 7.53-7.48 (m, 1H), 7.37-7.35 (m, 1H), 7.17-7.14 (m, 1H), 4.68 (d, J=8.0 Hz, 1H), 4.39 (s, 2H), 4.02-3.91 (m, 1H), 3.87 (s, 1H), 3.80 (d, J=4.8 Hz, 2H), 3.78-3.63 (m, 5H), 3.60 (s, 1H), 3.53 (d, J=5.2 Hz, 1H), 3.49-3.40 (m, 2H), 3.40-3.32 (m, 2H), 3.28-3.19 (m, 1H), 3.18-3.08 (m, 2H), 2.95-2.81 (m, 2H), 2.06-1.92 (m, 3H), 1.89 (s, 6H), 1.85-1.70 (m, 7H), 1.69-1.55 (m, 3H), 1.54-1.46 (m, 1H), 1.28-1.21 (m, 2H), 1.18 (s, 4H), 0.98-0.95 (m, 2H). ¹⁹F NMR (376 MHz, METHANOL-d4) δ=−120.764.

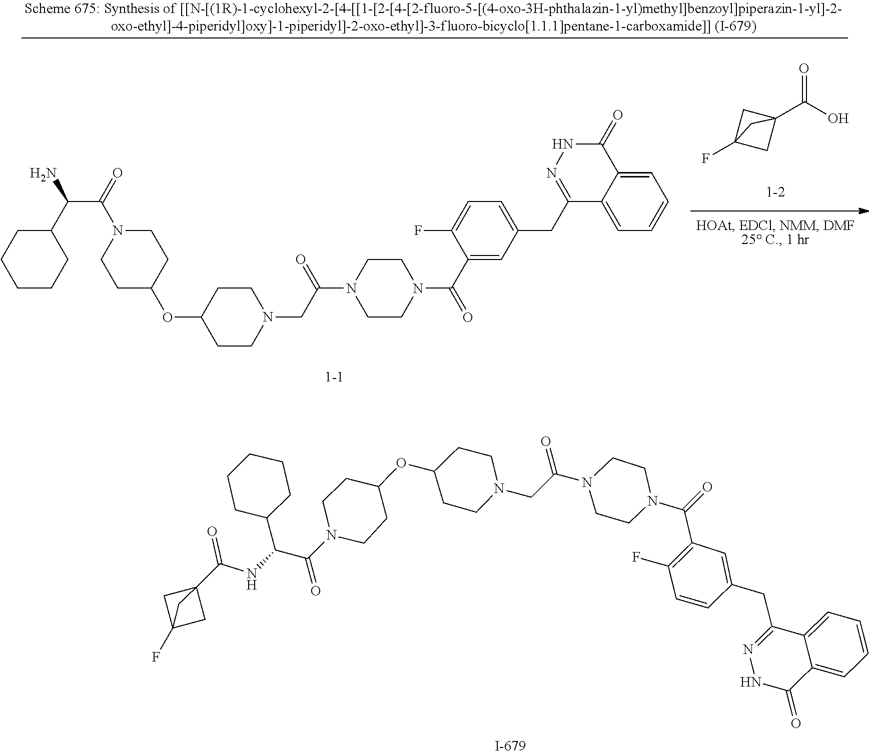 [SMILES: N[C@@H](C(=O)N1CCC(OC2CCN(CC(=O)N3CCN(C(=O)c4cc(Cc5n[nH]c(=O)c6ccccc56)ccc4F)CC3)CC2)CC1)C1CCCCC1 ; O=C(O)C12CC(F)(C1)C2 ; "HOAt, EDCl, NMM, DMF25° C., 1 hr" ; O=C(CN1CCC(OC2CCN(C(=O)[C@H](NC(=O)C34CC(F)(C3)C4)C3CCCCC3)CC2)CC1)N1CCN(C(=O)c2cc(Cc3n[nH]c(=O)c4ccccc34)ccc2F)CC1]

To a solution of Intermediate 1-2 (16.98 mg, 130.49 μmol, 1 eq) in DMF (1 mL) was added EDCI (75.05 mg, 391.47 μmol, 3 eq), HOAt (17.76 mg, 130.49 μmol, 18.25 μL, 1 eq), Intermediate 1-1 (100 mg, 130.49 μmol, 1 eq, HCl salt) and NMM (65.99 mg, 652.45 μmol, 71.73 μL, 5 eq). The mixture was stirred at 25° C. for 1 hr. The mixture was diluted with H₂O (1.5 mL) and extracted with EA (1.5 mL*2). The combined organic layers were washed with brine 1 mL (1 mL*1), dried over Na₂SO₄, filtered and concentrated under reduced pressure to give a residue. The residue was purified by prep-HPLC (column: CD01-Phenomenex luna C18 150*25*10 um; mobile phase: [water(FA)-ACN]; gradient: 10%-40% B over 11 min). The eluent was concentrated under reduced pressure to remove ACN and then lyophilized to give I-679 (48.16 mg, 54.23 μmol, 41.56% yield, 100% purity, FA salt) as a white solid. LCMS (Method D): Rt=0.377 min, [M+H]⁺=842.4. SFC: Rt=1.370 min. ¹H NMR (400 MHz, METHANOL-d4) δ=8.46-8.40 (m, 1H), 8.37 (d, J=7.6 Hz, 1H), 7.98-7.92 (m, 1H), 7.91-7.79 (m, 2H), 7.50 (s, 1H), 7.40-7.32 (m, 1H), 7.21-7.13 (m, 1H), 4.72-4.65 (m, 1H), 4.39 (s, 2H), 4.04-3.91 (m, 1H), 3.90-3.58 (m, 9H), 3.57-3.36 (m, 4H), 3.28-3.18 (m, 1H), 3.17-2.99 (m, 2H), 2.92-2.66 (m, 2H), 2.36-2.19 (m, 6H), 2.06-1.40 (m, 15H), 1.33-1.12 (m, 3H), 1.08-0.90 (m, 2H). ¹⁹F NMR (376 MHz, METHANOL-d4) δ=−120.787, −151.477.

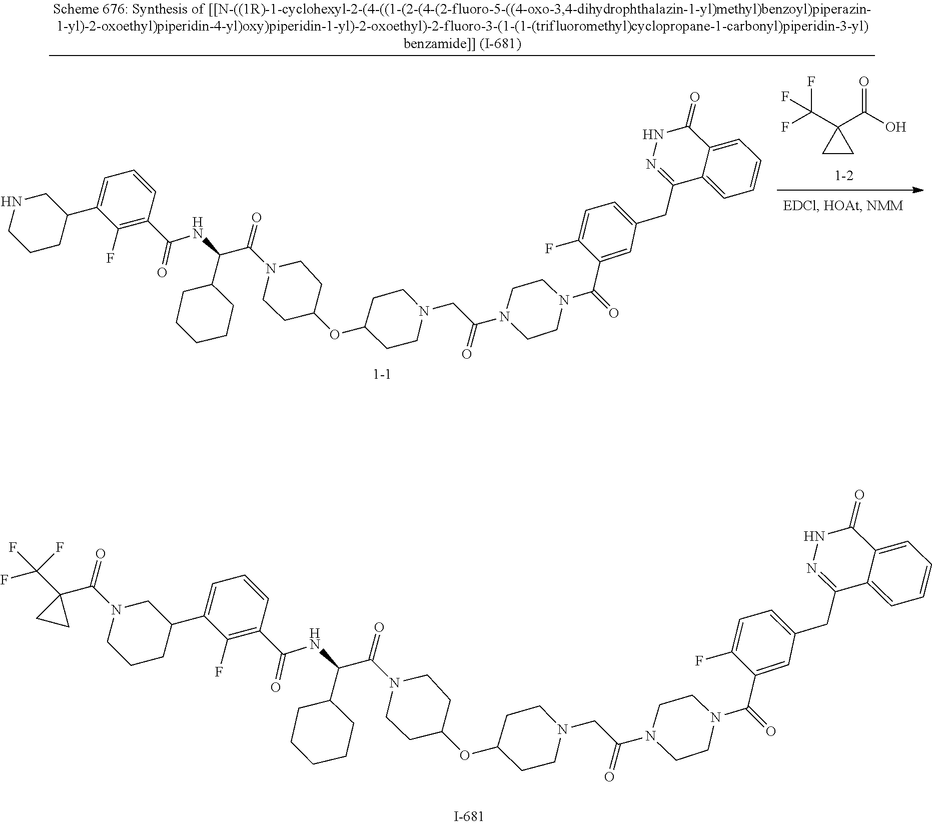

To a solution of Intermediate 1-1 (0.1 g, 102.93 μmol, 1 eq, HCl salt) and Intermediate 1-2 (19.03 mg, 123.51 μmol, 1.2 eq) in DMF (1 mL) was added EDCI (59.19 mg, 308.78 μmol, 3 eq), HOAt (14.01 mg, 102.93 μmol, 14.40 μL, 1 eq) and NMM (52.05 mg, 514.63 μmol, 56.58 μL, 5 eq). The mixture was stirred at 25° C. for 0.5 hr. The reaction mixture was filtered and concentrated under reduced pressure to give a residue. The residue was purified by prep-HPLC (column: CD01-Phenomenex luna C18 150*25*10 um; mobile phase: [water (FA)-ACN]; gradient: 22%-52% B over 10 min) and the eluent was concentrated to remove ACN and lyophilized to give I-681 (44.47 mg, 39.80 μmol, 38.67% yield, 100% purity, FA salt) as a white solid. LCMS (Method D): Retention time: 0.402 min, [M+H]⁺=1071.6. SFC: Retention time: 2.261 min, 2.693 min. ¹H NMR (400 MHz, METHANOL-d4) δ=8.44 (s, 1H), 8.37 (d, J=7.2 Hz, 1H), 7.95 (d, J=6.4 Hz, 1H), 7.91-7.80 (m, 2H), 7.59-7.48 (m, 3H), 7.37 (s, 1H), 7.30-7.23 (m, 1H), 7.17 (s, 1H), 4.96 (d, J=7.6 Hz, 1H), 4.65-4.43 (m, 2H), 4.39 (s, 2H), 4.09-3.86 (m, 2H), 3.86-3.74 (m, 4H), 3.67 (d, J=16.4 Hz, 5H), 3.60-3.56 (m, 1H), 3.55-3.46 (m, 3H), 3.27 (s, 1H), 3.15-2.97 (m, 4H), 2.78-2.62 (m, 2H), 2.02 (d, J=11.6 Hz, 2H), 1.94 (s, 3H), 1.90 (s, 2H), 1.87-1.77 (m, 5H), 1.76-1.60 (m, 6H), 1.58-1.48 (m, 1H), 1.36 (s, 2H), 1.32-1.21 (m, 5H), 1.19-1.10 (m, 2H). ¹⁹F NMR (377 MHz, METHANOL-d₄) δ=−68.91 (br d, J=8.5 Hz, 3F), −120.75 (br d, J=22.6 Hz, 1F), −121.43-−121.73 (m, 1F).

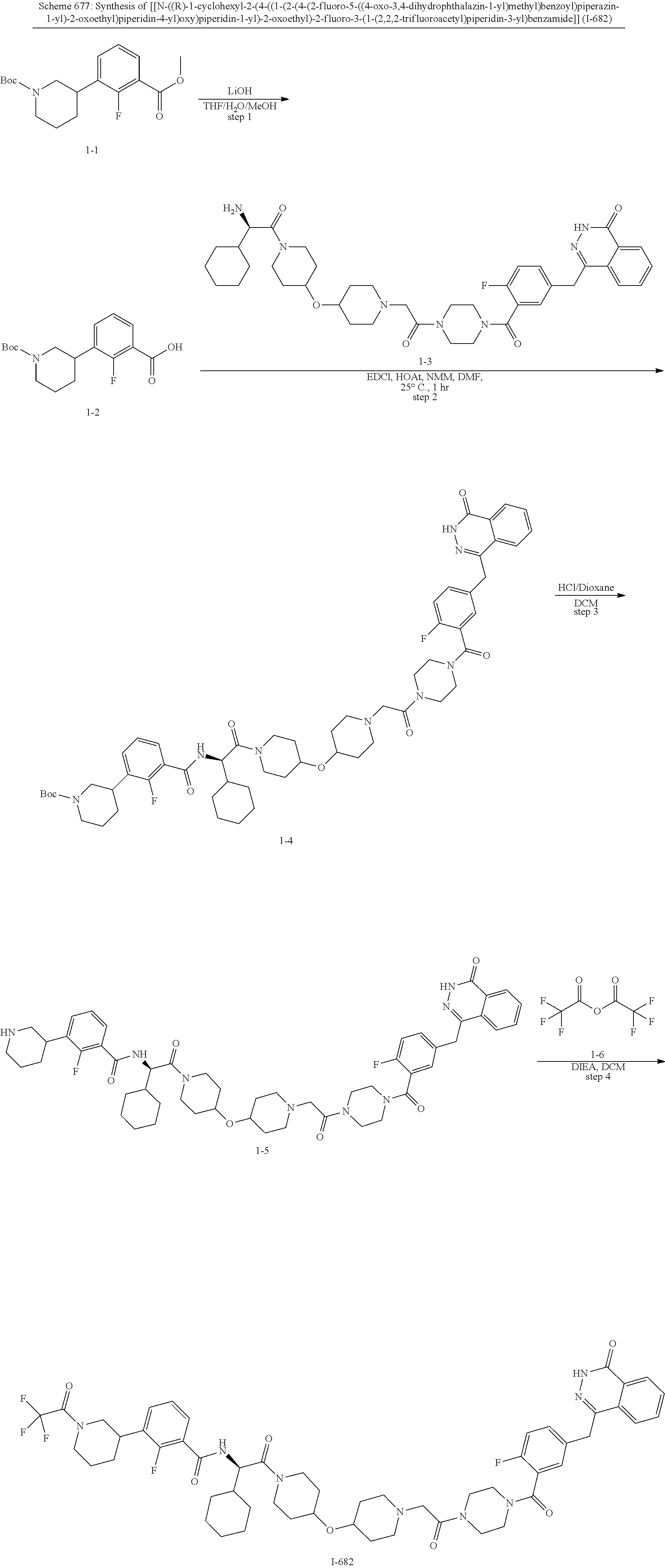

### Step 1: Synthesis of Intermediate 1-2.

To a solution of Intermediate 1-1 (200 mg, 592.79 μmol, 1 eq) in THF (1 mL), MeOH (1 mL) and H₂O (1 mL) was added LiOH·H₂O (49.75 mg, 1.19 mmol, 2 eq). The mixture was stirred at 25° C. for 1 hr. The pH of the aqueous phase was adjusted to 7 with HCl, and then the mixture was extracted with DCM (10 mL*3). The combined organic phase was dried by Na₂SO₄, filtered and concentrated. Intermediate 1-2 (100 mg, 309.25 μmol, 52.17% yield) was obtained as a white solid. LCMS (Method D): Retention time: 0.386 min, [M+Na]⁺=346.1.

### Step 2: Synthesis of Intermediate 1-4.

To a solution of Intermediate 1-3 (100 mg, 130.49 μmol, 1 eq, HCl salt) and Intermediate 1-2 (42.20 mg, 130.49 μmol, 1 eq) in DMF (1 mL) was added EDCI (50.03 mg, 260.98 μmol, 2 eq), HOAt (17.76 mg, 130.49 μmol, 18.25 μL, 1 eq) and NMM (65.99 mg, 652.45 μmol, 71.73 μL, 5 eq). The mixture was stirred at 25° C. for 1 hr. The reaction mixture was diluted with H₂O (2 mL) and then extracted with DCM (4 mL*3). The combined organic phase was dried by Na₂SO₄, filtered and concentrated. The residue was purified by flash silica gel chromatography (ISCO; 12 g SepaFlash Silica Flash Column, Eluent of 0˜100% Ethyl acetate/Petroleum ether gradient @40 mL/min). The eluent was concentrated to give product. Intermediate 1-4 (100 mg, 96.60 μmol, 74.03% yield) was obtained as a white solid. LCMS (Method D): Retention time: 0.414 min, [M+H]⁺=1035.7.

### Step 3: Synthesis of Intermediate 1-5.

A mixture of Intermediate 1-4 (100 mg, 96.60 μmol, 1 eq) and HCl/dioxane (2 M, 1 mL, 20.70 eq) was stirred at 25° C. for 1 hr. The reaction mixture was concentrated under reduced pressure to give a residue. The crude product was used in the next step. Intermediate 1-5 (100 mg, crude, HCl salt) was obtained as a white solid. LCMS (Method D): Retention time: 0.315 min, [M+H]⁺=935.6.

### Step 4: Synthesis of I-682.

To a solution of Intermediate 1-5 (100 mg, 102.93 μmol, 1 eq, HCl salt) and Intermediate 1-6 (25.94 mg, 123.51 μmol, 17.17 μL, 1.2 eq) in DCM (1 mL) was added DIEA (26.60 mg, 205.85 mol, 35.86 μL, 2 eq). The mixture was stirred at 25° C. for 1 hr. To the reaction mixture was added H₂O (2 mL) and then the mixture was extracted with DCM (2 mL*3). The combined organic phase was washed, dried by Na₂SO₄, filtered and concentrated. The crude product was purified by reversed phase chromatography (0.1% FA condition), the eluent was concentrated to remove ACN and lyophilized to give product. I-682 (22.71 mg, 21.08 μmol, 20.48% yield, 100.000% purity, FA salt) was obtained as a white solid. LCMS (Method D): Retention time: 0.408 min, [M+H]⁺=1031.6. SFC: Retention time: 2.097 min, 2.512 min. ¹H NMR (400 MHz, METHANOL-d4) δ=8.56-8.46 (m, 1H), 8.39 (d, J=7.6 Hz, 1H), 7.97 (d, J=7.2 Hz, 1H), 7.94-7.81 (m, 2H), 7.65-7.45 (m, 3H), 7.40 (s, 1H), 7.33-7.25 (m, 1H), 7.19 (t, J=8.8 Hz, 1H), 4.98 (d, J=8.0 Hz, 1H), 4.61-4.54 (m, 1H), 4.41 (s, 2H), 4.17-3.99 (m, 2H), 3.97-3.75 (m, 4H), 3.70 (s, 2H), 3.67-3.57 (m, 2H), 3.57-3.46 (m, 4H), 3.42 (s, 3H), 3.29-3.25 (m, 1H), 3.23-3.04 (m, 2H), 3.00-2.89 (m, 2H), 2.66-2.42 (m, 2H), 2.13-2.05 (m, 1H), 2.04-1.92 (m, 5H), 1.92-1.75 (m, 4H), 1.74-1.60 (m, 5H), 1.59-1.41 (m, 2H), 1.40-1.06 (m, 6H). ¹⁹F NMR (377 MHz, METHANOL-d₄), δ=−70.13-−70.62 (m, 3F), −120.76 (br d, J=17.0 Hz, 1F), −121.46-−121.91 (m, 1F).

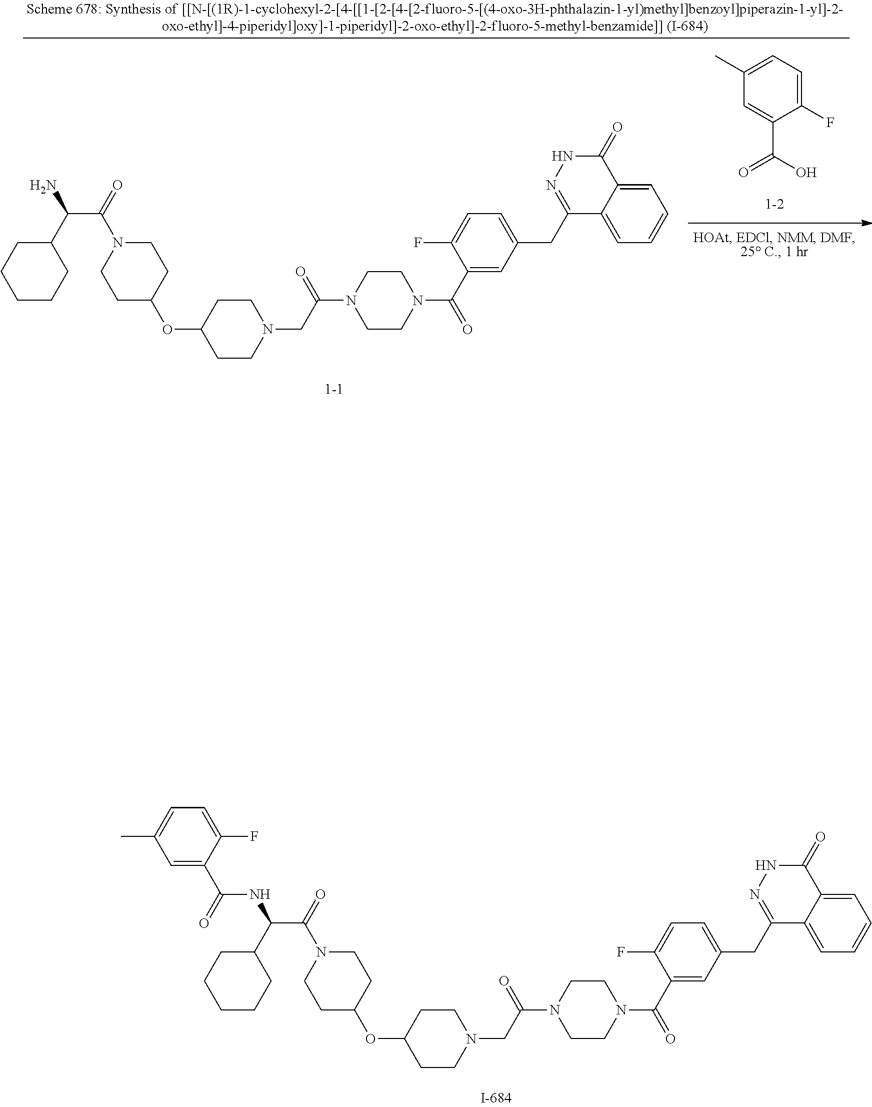 [SMILES: N[C@@H](C(=O)N1CCC(OC2CCN(CC(=O)N3CCN(C(=O)c4cc(Cc5n[nH]c(=O)c6ccccc56)ccc4F)CC3)CC2)CC1)C1CCCCC1 ; Cc1ccc(F)c(C(=O)O)c1 ; "HOAt, EDCl, NMM, DMF,25° C., 1 hr" ; Cc1ccc(F)c(C(=O)N[C@@H](C(=O)N2CCC(OC3CCN(CC(=O)N4CCN(C(=O)c5cc(Cc6n[nH]c(=O)c7ccccc67)ccc5F)CC4)CC3)CC2)C2CCCCC2)c1]

### Step 1: Synthesis of I-684.

To a solution of Intermediate 1-1 (100 mg, 130.49 μmol, 1 eq, HCl salt) in DMF (1 mL) was added Intermediate 1-2 (20.11 mg, 130.49 μmol, 1 eq), EDCI (75.05 mg, 391.47 μmol, 3 eq), HOAt (17.76 mg, 130.49 μmol, 18.25 μL, 1 eq) and NMM (65.99 mg, 652.45 μmol, 71.73 μL, 5 eq). The mixture was stirred at 25° C. for 1 hr. The reaction mixture was diluted with H₂O (1 mL) and extracted with EA (2 mL*2). The combined organic layers were washed with brine (2 mL*1), dried over Na₂SO₄, filtered and concentrated under reduced pressure to give a residue. The crude product was purified by prep-HPLC (column: CD01-Phenomenex luna C18 150*25*10 um; mobile phase: [water (FA)-ACN]; gradient: 22%-52% B over 10 min). The eluent was concentrated under reduced pressure to remove ACN and then lyophilized to give I-684 (63.77 mg, 69.52 μmol, 53.27% yield, 99.422% purity, FA salt) as a white solid. LCMS (Method D): Retention time: 0.403 min, [M+H]⁺=866.5. SFC: Retention time: 1.814 min. ¹H NMR (400 MHz, METHANOL-d₄) δ=8.61-8.45 (m, 1H), 8.37 (d, J=7.6 Hz, 1H), 7.98-7.92 (m, 1H), 7.91-7.80 (m, 2H), 7.58-7.46 (m, 2H), 7.41-7.30 (m, 2H), 7.20-7.05 (m, 2H), 4.97 (d, J=7.2 Hz, 1H), 4.39 (s, 2H), 4.08-3.96 (m, 1H), 3.95-3.83 (m, 1H), 3.82-3.74 (m, 3H), 3.72-3.56 (m, 4H), 3.55-3.45 (m, 4H), 3.42-3.37 (m, 1H), 3.36-3.33 (m, 1H), 3.28-3.20 (m, 1H), 2.99-2.84 (m, 2H), 2.63-2.40 (m, 2H), 2.35 (s, 3H), 2.01-1.88 (m, 3H), 1.87-1.75 (m, 5H), 1.74-1.60 (m, 5H), 1.57-1.45 (m, 1H), 1.35-1.20 (m, 3H), 1.18-1.07 (m, 2H). ¹⁹F NMR (377 MHz, MeOD-d₄) δ=−120.753, −120.960.

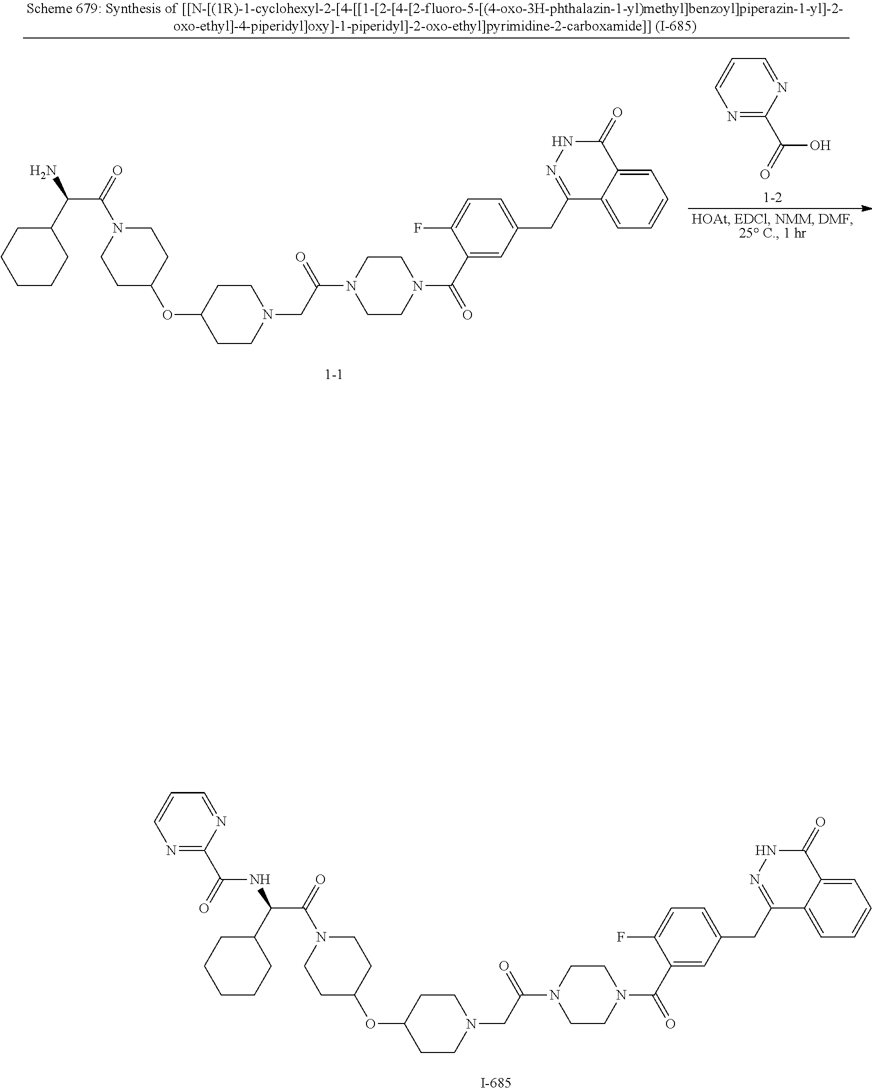 [SMILES: N[C@@H](C(=O)N1CCC(OC2CCN(CC(=O)N3CCN(C(=O)c4cc(Cc5n[nH]c(=O)c6ccccc56)ccc4F)CC3)CC2)CC1)C1CCCCC1 ; O=C(O)c1ncccn1 ; "HOAt, EDCl, NMM, DMF,25° C., 1 hr" ; O=C(N[C@@H](C(=O)N1CCC(OC2CCN(CC(=O)N3CCN(C(=O)c4cc(Cc5n[nH]c(=O)c6ccccc56)ccc4F)CC3)CC2)CC1)C1CCCCC1)c1ncccn1]

To a solution of Intermediate 1-1 (80 mg, 104.39 μmol, 1 eq, HCl salt) in DMF (1 mL) was added Intermediate 1-2 (12.95 mg, 104.39 μmol, 1 eq), EDCI (60.04 mg, 313.18 μmol, 3 eq), HOAt (14.21 mg, 104.39 μmol, 14.60 μL, 1 eq) and NMM (52.79 mg, 521.96 μmol, 57.39 μL, 5 eq). The mixture was stirred at 25° C. for 1 hr. The reaction mixture was diluted with H₂O (1 mL) and extracted with EA (2 mL*2). The combined organic layers were washed with brine (2 mL*1), dried over Na₂SO₄, filtered and concentrated under reduced pressure to give a residue. The crude product was purified by prep-HPLC (column: CD01-Phenomenex luna C18 150*25*10 um; mobile phase: [water (FA)-ACN]; gradient: 10%-40% B over 8 min). The eluent was concentrated under reduced pressure to remove ACN and then lyophilized to give I-685 (21.53 mg, 24.41 μmol, 23.38% yield, 100% purity, FA salt) as a white solid. LCMS (Method D): Retention time: 0.352 min, [M+H]⁺=836.3. SFC: Retention time: 1.809 min. ¹H NMR (400 MHz, METHANOL-d₄) δ=8.96 (d, J=4.9 Hz, 2H), 8.54-8.42 (m, 1H), 8.41-8.31 (m, 1H), 7.98-7.92 (m, 1H), 7.91-7.80 (m, 2H), 7.69-7.62 (m, 1H), 7.54-7.46 (m, 1H), 7.42-7.32 (m, 1H), 7.24-7.10 (m, 1H), 5.10-4.97 (m, 1H), 4.39 (s, 2H), 4.09-3.96 (m, 1H), 3.84-3.56 (m, 9H), 3.55-3.44 (m, 3H), 3.38-3.32 (m, 2H), 3.29-3.22 (m, 1H), 3.11-2.95 (m, 2H), 2.84-2.57 (m, 2H), 2.03-1.90 (m, 3H), 1.89-1.60 (m, 10H), 1.58-1.43 (m, 1H), 1.33-1.06 (m, 5H). ¹⁹F NMR (377 MHz, MeOD-d₄) δ=−120.764.

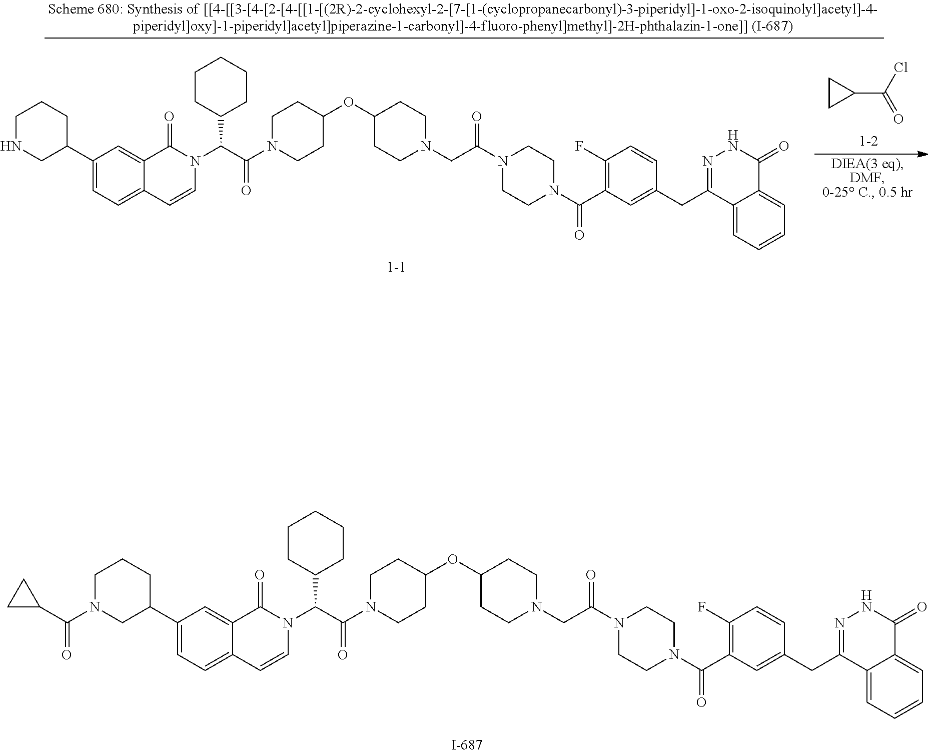 [SMILES: O=C(CN1CCC(OC2CCN(C(=O)[C@@H](C3CCCCC3)n3ccc4ccc(C5CCCNC5)cc4c3=O)CC2)CC1)N1CCN(C(=O)c2cc(Cc3n[nH]c(=O)c4ccccc34)ccc2F)CC1 ; O=C(Cl)C1CC1 ; "DIEA(3 eq),DMF,0-25° C., 0.5 hr" ; O=C(CN1CCC(OC2CCN(C(=O)[C@@H](C3CCCCC3)n3ccc4ccc(C5CCCN(C(=O)C6CC6)C5)cc4c3=O)CC2)CC1)N1CCN(C(=O)c2cc(Cc3n[nH]c(=O)c4ccccc34)ccc2F)CC1]

To a solution of Intermediate 1-1 (40 mg, 42.50 μmol, 1 eq) and DIEA (16.48 mg, 127.50 μmol, 22.21 μL, 3 eq) in DMF (4 mL) was added Intermediate 1-2 (8.89 mg, 85.00 μmol, 7.71 μL, 2 eq) at 0° C. The mixture was stirred at 25° C. for 0.5 hr. The mixture was diluted with water (4 mL), extracted with EtOAc (10 mL*3) and washed with brine (2 mL*3). The organic layer was dried with anhydrous Na₂SO₄, filtered and concentrated under vacuum. The residue was purified by prep-HPLC (column: Waters Xbridge 150*25 mm*5 um; mobile phase: [water (NH₄HCO₃)-ACN]; gradient: 40%-70% B over 15 min) and dried by lyophilization. I-687 (10 mg, 9.91 μmol, 23.31% yield, 100% purity) as a white solid was obtained. LCMS (Method G): Rt=0.718 min, [M+H]⁺=1009.6. SFC: Rt=3.252 min, 3.992 min. ¹H NMR (400 MHz, DMSO-d₆) δ=12.59 (br s, 1H), 8.30-8.21 (m, 1H), 8.19-8.08 (m, 1H), 7.97-7.95 (m, 1H), 7.91-7.79 (m, 2H), 7.76-7.59 (m, 2H), 7.46-7.33 (m, 3H), 7.26-7.20 (m, 1H), 6.71-6.65 (m, 1H), 5.80-5.62 (m, 1H), 4.49-4.38 (m, 1H), 4.37-4.26 (m, 3H), 3.99-3.79 (m, 2H), 3.66-3.53 (m, 4H), 3.45 (br d, J=17.6 Hz, 2H), 3.21-3.00 (m, 6H), 2.73-2.53 (m, 4H), 2.17-1.96 (m, 5H), 1.88-1.50 (m, 12H), 1.46-1.20 (m, 5H), 1.11 (br s, 5H), 0.93-0.63 (m, 6H). ¹⁹F NMR (377 MHz, DMSO-d₆) δ=−119.20-−120.37 (m, 1F).

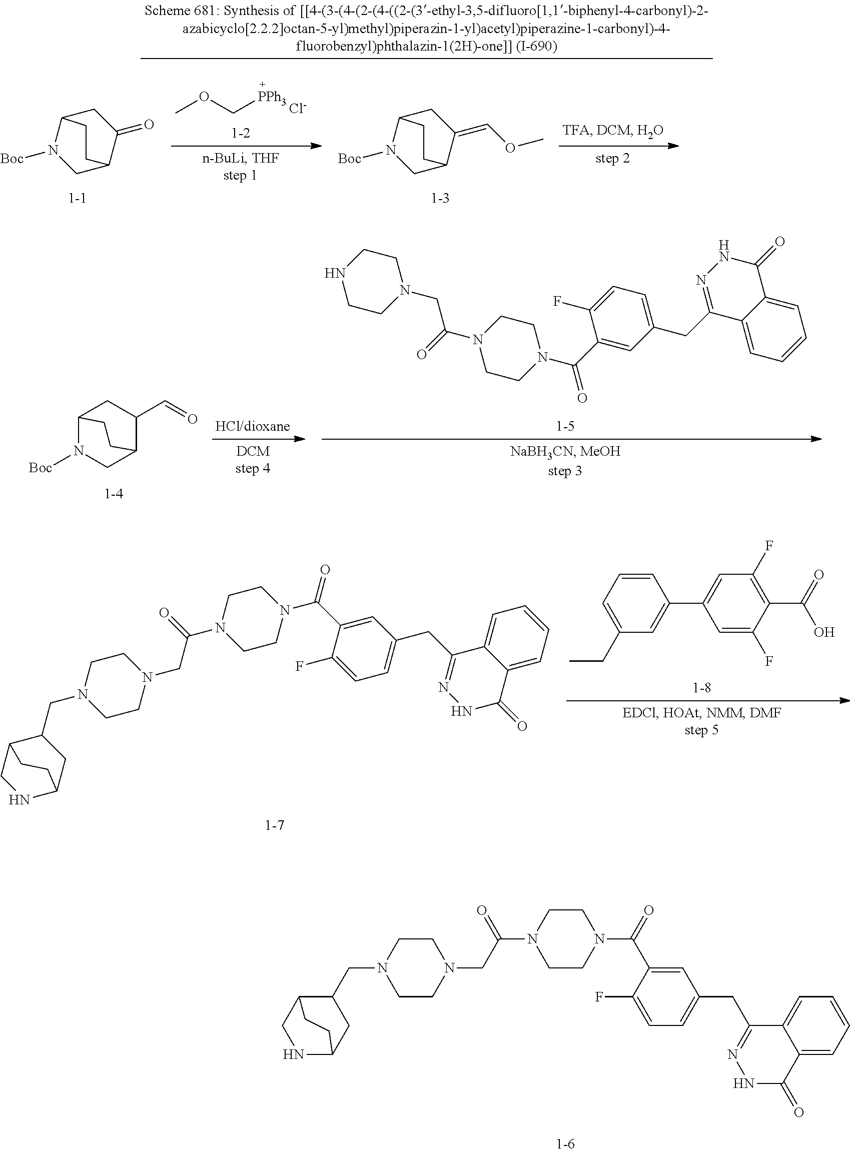

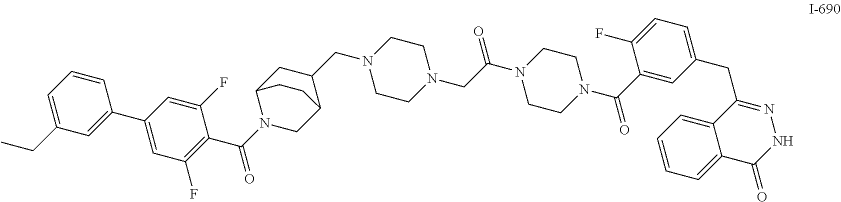 [SMILES: CCc1cccc(-c2cc(F)c(C(=O)N3CC4CCC3CC4CN3CCN(CC(=O)N4CCN(C(=O)c5cc(Cc6n[nH]c(=O)c7ccccc67)ccc5F)CC4)CC3)c(F)c2)c1]

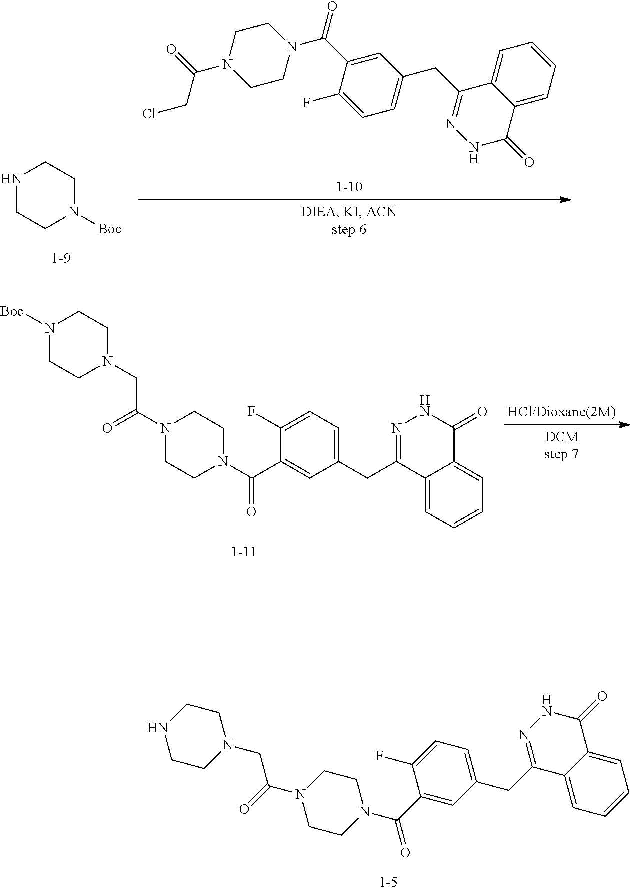 [SMILES: CC(C)(C)OC(=O)N1CCNCC1 ; O=C(CCl)N1CCN(C(=O)c2cc(Cc3n[nH]c(=O)c4ccccc34)ccc2F)CC1 ; CC(C)(C)OC(=O)N1CCN(CC(=O)N2CCN(C(=O)c3cc(Cc4n[nH]c(=O)c5ccccc45)ccc3F)CC2)CC1 ; O=C(CN1CCNCC1)N1CCN(C(=O)c2cc(Cc3n[nH]c(=O)c4ccccc34)ccc2F)CC1]

### Step 1: Synthesis of Intermediate 1-3

To a solution of Intermediate 1-2 (4.56 g, 13.32 mmol, 1.5 eq) in THF (10 mL) was added n-BuLi (2.5 M, 5.33 mL, 1.5 eq) at −10° C. under N₂ atmosphere. The reaction mixture was stirred at 0° C. for 1 h. A solution of Intermediate 1-1 (2 g, 8.88 mmol, 1 eq) in THF (10 mL) was added to the reaction solution at −20° C. dropwise. The mixture was stirred for 2 h at 25° C. under N₂ atmosphere. The mixture was quenched with sat. NH₄Cl (40 mL) under N₂ atmosphere and extracted with EA (20 mL*3). The combined organic layers were dried over Na₂SO₄, filtered and concentrated under reduced pressure to give the residue. The crude was purified by Flash Silica-gel chromatography (ISCO; 40 g SepaFlash Silica Flash Column, Eluent of 16-20% EA/PE) and the eluent was combined and concentrated to give the target product. Intermediate 1-3 (1.3 g, 5.13 mmol, 57.80% yield) was obtained as a colorless oil. ¹H NMR (400 MHz, CHLOROFORM-d) δ=6.01-5.70 (m, 1H), 4.31-3.99 (m, 1H), 3.63-3.50 (m, 3H), 3.45-3.26 (m, 2H), 3.06-2.88 (m, 1H), 2.64-2.12 (m, 2H), 2.00-1.60 (m, 4H), 1.46 (s, 9H).

### Step 2: Synthesis of Intermediate 1-4

To a solution of Intermediate 1-3 (1.3 g, 5.13 mmol, 1 eq) in DCM (11.7 mL) was added H₂O (3.9 mL) and TFA (3.51 g, 30.79 mmol, 2.29 mL, 6 eq). The resulting mixture was stirred at 25° C. for 2 hrs. The reaction mixture was quenched by saturated NaHCO₃ (20 mL), and extracted with DCM (5 mL*3). The organic phase was combined and concentrated to afford the crude. The crude was purified by flash silica gel chromatography (ISCO; 12 g SepaFlash Silica Flash Column, Eluent of 15˜25% Petroleum ether/Ethyl acetate gradient @20 mL/min). The eluent was combined and concentrated to afford the target product. Intermediate 1-4 (0.4 g, 1.59 mmol, 30.94% yield, 95% purity) was obtained as a colorless oil. LCMS (Method E): Rt=0.512 min, [M+H-tBu]⁺=184.4.

### Step 3: Synthesis of Intermediate 1-6

To a solution of Intermediate 1-4 (0.4 g, 1.67 mmol, 1 eq) in MeOH (40 mL) was added Intermediate 1-5 (823.27 mg, 1.67 mmol, 1 eq, 2 HCl salt). The resulting mixture was stirred at 25° C. for 1.5 hrs. Then NaBH₃CN (210.08 mg, 3.34 mmol, 2 eq) was introduced into the system. The system was kept at 25° C. and stirred for another 16 hrs. The reaction mixture was quenched by addition of NH₄Cl (aq.) (30 mL). The mixture was then concentrated to remove MeOH (40 mL) and then basified by addition of NaHCO₃. The suspension was extracted by DCM (50 mL*3, 25% V/V MeOH). The organic phases were combined and concentrated to afford the crude. The crude was purified by prep-HPLC (FA condition), and the eluent was combined and basified. The resulting suspension was extracted by DCM (30 mL*4, 25% V/V MeOH), the organic layer was combined and concentrated to afford the target product. Intermediate 1-6 (0.9 g, 1.23 mmol, 73.71% yield, 98% purity) was obtained as off-white solid. LCMS (Method E): Rt=0.444 min, [M+H]⁺=716.2. ¹H NMR (400 MHz, DMSO-d₆) δ=12.68-12.47 (m, 1H), 8.26 (d, J=7.6 Hz, 1H), 7.99-7.94 (m, 1H), 7.92-7.86 (m, 1H), 7.85-7.79 (m, 1H), 7.48-7.40 (m, 1H), 7.40-7.32 (m, 1H), 7.28-7.18 (m, 1H), 4.35-4.32 (m, 2H), 3.86-3.71 (m, 2H), 3.62 (d, J=7.2 Hz, 6H), 3.27-2.98 (m, 6H), 2.47-2.01 (m, 10H), 1.99-1.46 (m, 7H), 1.40-1.35 (m, 9H), 1.25-1.11 (m, 1H).

### Step 4: Synthesis of Intermediate 1-7

Intermediate 1-6 (1.5 g, 2.10 mmol, 1 eq) was suspended in HCl/dioxane (25 mL) and stirred at 25° C. for 1 hr. The reaction mixture was concentrated directly to afford the crude. The crude was used in the next step without purification. Intermediate 1-7 (1.4 g, crude, 3HCl salt) was obtained as a white solid. LCMS (Method E): Rt=0.361 min, [M+H]⁺=616.2. ¹H NMR (400 MHz, DMSO-d₆) δ=12.70-12.46 (m, 1H), 8.30-8.23 (m, 1H), 8.01-7.78 (m, 3H), 7.52-7.43 (m, 1H), 7.42-7.32 (m, 1H), 7.30-7.20 (m, 1H), 5.63-4.88 (m, 3H), 4.73-4.30 (m, 4H), 3.95-3.63 (m, 8H), 3.50-2.89 (m, 12H), 2.42-1.27 (m, 9H).

### Step 5: Synthesis of I-690

To a solution of Intermediate 1-7 (1.4 g, 2.15 mmol, 1 eq, 3HCl salt) in DMF (30 mL) was added Intermediate 1-8 (562.94 mg, 2.15 mmol, 1 eq), NMM (1.74 g, 17.17 mmol, 1.89 mL, 8 eq), HOAt (292.17 mg, 2.15 mmol, 300.28 μL, 1 eq) and EDCI (1.23 g, 6.44 mmol, 3 eq). The resulting mixture was stirred at 25° C. for 1 hr. The reaction mixture was diluted by water (150 mL) and extracted by EA (30 mL*3), the organic phase was combined and concentrated to afford the crude. The crude was combined with another lot of material (100 mg) and purified by Prep-HPLC (FA condition). The eluent was combined and lyophilized to afford the target product. I-690 (0.73279 g, 809.50 μmol, 37.71% yield, 95% purity, FA salt) was obtained as a white solid. LCMS (Method E): Rt=1.229 min, 1.248 min, [M+H]⁺=860.7. SFC: Rt=2.332 min, 2.669 min, 3.240 min, 4.106 min. ¹H NMR (400 MHz, DMSO-d₆) δ=12.60 (s, 1H), 8.31-8.22 (m, 1H), 8.16 (s, 1H), 8.00-7.93 (m, 1H), 7.92-7.86 (m, 1H), 7.85-7.79 (m, 1H), 7.67-7.51 (m, 4H), 7.48-7.33 (m, 3H), 7.32-7.18 (m, 2H), 4.47-4.25 (m, 2H), 3.78-3.04 (m, 13H), 2.72-2.63 (m, 2H), 2.49-1.48 (m, 17H), 1.33-1.05 (m, 4H). ¹⁹F NMR (377 MHz, DMSO-d₆) δ=−114.03-−114.97 (m, 2F), −119.77 (s, 1F).

### Step 6: Synthesis of Intermediate 1-11

To a solution of Intermediate 1-10 (3.0 g, 6.77 mmol, 1 eq) and Intermediate 1-9 (1.26 g, 6.77 mmol, 1 eq) in ACN (30 mL) was added DIEA (3.50 g, 27.10 mmol, 4.72 mL, 4 eq) and KI (112.45 mg, 677.40 μmol, 0.1 eq). The mixture was stirred at 70° C. for 1 hr. The mixture was filtered, and the filtrate was concentrated under reduced pressure to afford the crude. The crude was purified by Flash Silica-gel chromatography (ISCO; 40 g SepaFlash Silica Flash Column, Eluent of 14-18% EA/PE) and the eluent was concentrated to give the target product. Intermediate 1-11 (2.2 g, 3.71 mmol, 54.80% yield) was obtained as a yellow oil. LCMS (Method E): Rt=0.421 min, [M+H]⁺=593.5.

### Step 7: Synthesis of Intermediate 1-5

Intermediate 1-11 (2.2 g, 2.00 mmol, 1 eq) was dissolved in HCl/dioxane (40 mL) (2M) and stirred for 2 hrs at 25° C. The suspension was concentrated to afford the crude product. After work-up, Intermediate 1-5 (1.8 g, crude, 2HCl salt) was obtained as a yellow oil. LCMS (Method G): Rt=0.491 min, [M+H]⁺=493.3.

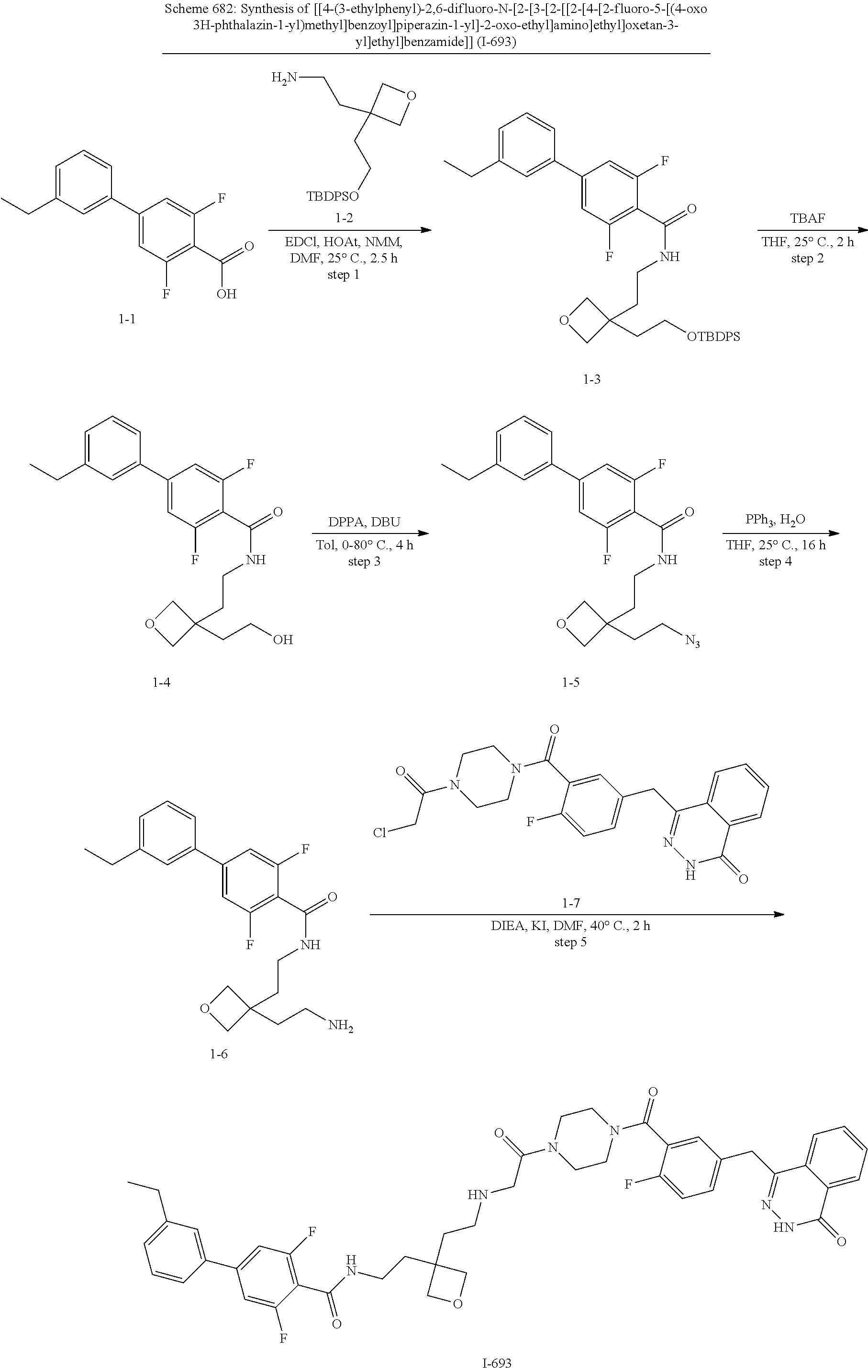

### Step 1: Synthesis of Intermediate 1-3

To a solution of Intermediate 1-1 (205.10 mg, 782.07 μmol, 1 eq) in DMF (6 mL) was added HOAt (212.89 mg, 1.56 mmol, 218.80 μL, 2 eq) and EDCI (299.85 mg, 1.56 mmol, 2 eq). The mixture was stirred at 25° C. for 0.5 hr. Then Intermediate 1-2 (300 mg, 782.07 μmol, 1 eq) and NMM (237.32 mg, 2.35 mmol, 257.96 μL, 3 eq) were added and the mixture was stirred at 25° C. for 2 hr. The mixture was poured into 50 mL of water and extracted with EA (50 mL*2). The combined organic phase was dried over anhydrous Na₂SO₄, filtered and concentrated in vacuum. The residue was purified by flash silica gel chromatography (ISCO; 40 g SepaFlash Silica Flash Column, Eluent of 0˜30% Ethyl acetate/Petroleum ether gradient @40 mL/min). Intermediate 1-3 (330 mg, 525.62 μmol, 67.21% yield) was obtained as a colorless gum. LCMS (Method E): Rt=0.7291 min, [M+H]⁺=628.3. ¹H NMR (400 MHz, CHLOROFORM-d) δ=7.74-7.62 (m, 4H), 7.45-7.36 (m, 9H), 7.31-7.28 (m, 1H), 7.22-7.17 (m, 2H), 6.13-5.88 (m, 1H), 4.54 (d, J=6.0 Hz, 2H), 4.47 (d, J=6.0 Hz, 2H), 3.77 (t, J=6.2 Hz, 2H), 3.49-3.37 (m, 2H), 2.74 (q, J=7.5 Hz, 2H), 2.12-2.00 (m, 4H), 1.30 (s, 3H), 1.05 (s, 9H).

### Step 2: Synthesis of Intermediate 1-4

To a solution of Intermediate 1-3 (330 mg, 525.62 μmol, 1 eq) in THF (5 mL) was added TBAF (1 M, 788.43 μL, 1.5 eq). The mixture was stirred at 25° C. for 2 hr. The mixture was concentrated in vacuum. The residue was purified by flash silica gel chromatography (ISCO; 20 g SepaFlash Silica Flash Column, Eluent of 0˜100% Ethyl acetate/Petroleum ether gradient @20 mL/min). Intermediate 1-4 (180 mg, 462.21 μmol, 87.94% yield) was obtained as a yellow gum. LCMS (Method G): Rt=0.576 min, [M+H]⁺=390.2. ¹H NMR (400 MHz, CHLOROFORM-d) δ=7.43-7.34 (m, 3H), 7.29 (br s, 1H), 7.19 (d, J=9.4 Hz, 2H), 6.38 (br s, 1H), 4.56-4.52 (m, 2H), 4.52-4.47 (m, 2H), 3.84 (t, J=6.3 Hz, 2H), 3.60-3.49 (m, 2H), 2.73 (q, J=7.6 Hz, 2H), 2.21-2.13 (m, 2H), 2.09 (t, J=6.3 Hz, 2H), 1.33-1.27 (m, 3H).

### Step 3: Synthesis of Intermediate 1-5

To a solution of Intermediate 1-4 (180 mg, 462.21 μmol, 1 eq) in toluene (5 mL) was added DBU (84.44 mg, 554.65 μmol, 83.60 μL, 1.2 eq) and DPPA (152.64 mg, 554.65 μmol, 119.72 μL, 1.2 eq) at 0° C. The mixture was stirred at 80° C. for 4 hr. The mixture was poured into 10 mL of water and extracted with EA (10 mL*2). The combined organic phase was dried over anhydrous Na₂SO₄, filtered and concentrated in vacuum. The residue was purified by flash silica gel chromatography (ISCO; 12 g SepaFlash Silica Flash Column, Eluent of 0˜50% Ethyl acetate/Petroleum ether gradient @20 mL/min). Intermediate 1-5 (130 mg, 313.67 μmol, 67.86% yield) was obtained as a white gum. LCMS (Method E): Rt=0.557 min, [M+Na]⁺=437.2. ¹H NMR (400 MHz, CHLOROFORM-d) δ=7.44-7.35 (m, 3H), 7.31-7.27 (m, 1H), 7.23-7.17 (m, 2H), 6.18 (br s, 1H), 4.51 (s, 4H), 3.56-3.43 (m, 4H), 2.73 (q, J=7.6 Hz, 2H), 2.17-2.03 (m, 4H), 1.30 (t, J=7.6 Hz, 3H).

### Step 4: Synthesis of Intermediate 1-6

To a solution of PPh₃ (123.41 mg, 470.51 μmol, 1.5 eq) in THF (2 mL) and H₂O (16.95 mg, 941.01 μmol, 16.95 μL, 3 eq) was added a solution of Intermediate 1-5 (130 mg, 313.67 μmol, 1 eq) in THF (2 mL). The mixture was stirred at 25° C. for 16 hr. The mixture was concentrated in vacuum. The crude product was purified by reversed-phase chromatography (0.1% FA condition). Intermediate 1-6 (60 mg, 154.46 μmol, 49.24% yield) was obtained as a white gum. LCMS (Method E): Rt=0.463 min, [M+H]⁺=389.2. ¹H NMR (400 MHz, CHLOROFORM-d) δ=7.39-7.32 (m, 3H), 7.25 (s, 1H), 7.15 (d, J=9.4 Hz, 2H), 6.81 (br s, 1H), 4.48 (s, 4H), 3.55-3.46 (m, 2H), 3.32-2.99 (m, 2H), 2.94-2.86 (m, 2H), 2.71 (q, J=7.6 Hz, 2H), 2.10-2.02 (m, 4H), 1.30-1.26 (m, 3H).

### Step 5: Synthesis of I-693

To a solution of Intermediate 1-6 (50 mg, 128.72 μmol, 1 eq), Intermediate 1-7 (39.90 mg, 90.10 μmol, 0.7 eq) and KI (42.73 mg, 257.43 μmol, 2 eq) in DMF (2.5 mL) was added DIEA (33.27 mg, 257.43 μmol, 44.84 μL, 2 eq). The mixture was stirred at 40° C. for 2 hr. The mixture was purified directly without work-up. The crude product was purified by reversed-phase chromatography (0.1% NH₃·H₂O). I-693 (13.98 mg, 17.59 μmol, 13.66% yield, 100% purity) was obtained as a white solid. LCMS (Method G): Rt=0.630 min, [M+H]⁺=795.4. ¹H NMR (400 MHz, METHANOL-d₄) δ=8.36 (br d, J=7.3 Hz, 1H), 7.97-7.90 (m, 1H), 7.89-7.78 (m, 2H), 7.55-7.42 (m, 3H), 7.41-7.26 (m, 5H), 7.18-7.10 (m, 1H), 4.60-4.42 (m, 3H), 4.40-4.33 (m, 2H), 3.86-3.73 (m, 2H), 3.73-3.65 (m, 2H), 3.64-3.52 (m, 3H), 3.50-3.40 (m, 4H), 3.37-3.34 (m, 2H), 2.83-2.65 (m, 4H), 2.24-1.93 (m, 3H), 1.83-1.72 (m, 1H), 1.31-1.23 (m, 3H). ¹⁹F NMR (377 MHz, METHANOL-d₄) δ=−115.18-−115.30 (m, 2F), −120.62-−120.87 (m, 1F).

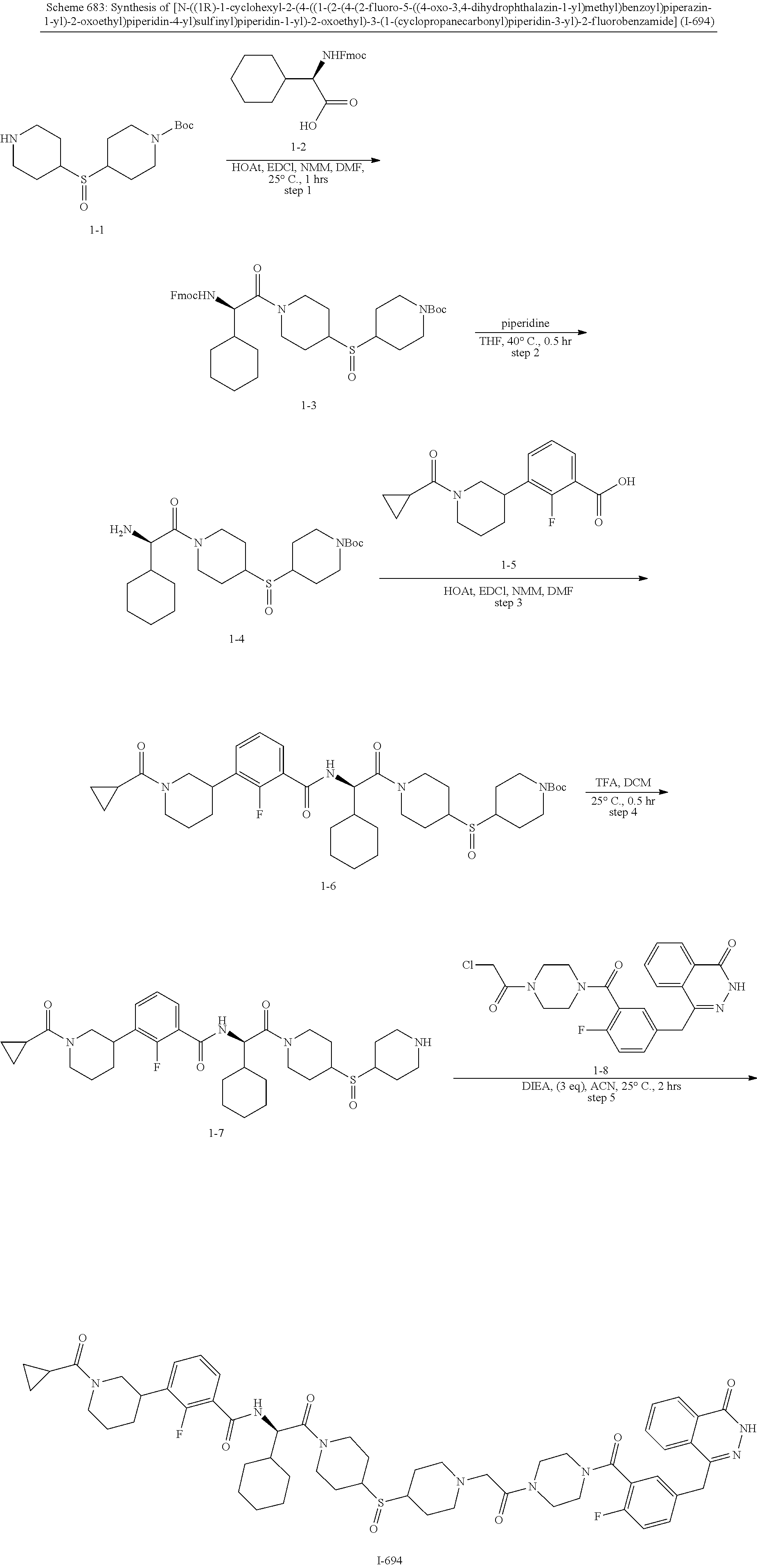

### Step 1: Synthesis of Intermediate 1-3.

To a solution of Intermediate 1-2 (500 mg, 1.32 mmol, 500.00 μL, 1 eq) and Intermediate 1-1 (417.00 mg, 1.32 mmol, 1 eq) in DMF (5 mL) was added HOAt (179.35 mg, 1.32 mmol, 184.33 μL, 1 eq), EDCI (757.82 mg, 3.95 mmol, 3 eq) and NMM (666.41 mg, 6.59 mmol, 724.36 μL, 5 eq). The mixture was stirred at 25° C. for 1 hr. The reaction mixture was diluted with H₂O (10 mL) and extracted with EA (10 mL*3). The combined organic layers were washed with brine (10 mL*3), dried over Na₂SO₄, filtered and concentrated under reduced pressure to give a residue. The residue was purified by flash silica gel chromatography (ISCO; 20 g SepaFlash Silica Flash Column, Eluent of 0˜100% Ethyl acetate/Petroleum ether gradient @60 mL/min), and the eluent was concentrated under reduced pressure to give a residue. Intermediate 1-3 (600 mg, 814.21 μmol, 61.79% yield, 91.991% purity) was obtained as a colorless oil. LCMS (Method D): Retention time: 0.473 min, [M+H]⁺=678.4. SFC: Retention time: 1.105, 1.342 min. ¹H NMR (400 MHz, CHLOROFORM-d) δ=7.77 (d, J=7.6 Hz, 2H), 7.66-7.55 (m, 2H), 7.47-7.38 (m, 2H), 7.36-7.30 (m, 2H), 5.56 (d, J=8.0 Hz, 1H), 4.79-4.18 (m, 7H), 3.34-3.17 (m, 1H), 2.97-2.66 (m, 5H), 1.93-1.66 (m, 11H), 1.60 (s, 5H), 1.47 (s, 9H), 1.20-0.99 (m, 4H).

### Step 2: Synthesis of Intermediate 1-4.

To a solution of Intermediate 1-3 (400 mg, 590.07 μmol, 1 eq) in THF (4 mL) was added piperidine (50.24 mg, 590.07 μmol, 58.27 μL, 1 eq). The mixture was stirred at 40° C. for 0.5 hr. The reaction mixture was concentrated under reduced pressure to give a residue. The crude product was purified by reversed-phase chromatography (basic condition), and the eluent was concentrated and lyophilized to give the desired product. Intermediate 1-4 (270 mg, 569.61 μmol, 96.53% yield, 96.127% purity) was obtained as a white solid. LCMS (Method D): Retention time: 0.307 min, [M+H]⁺=456.3. SFC: Retention time: 0.831, 1.490 min. ¹H NMR (400 MHz, CHLOROFORM-d4) δ=4.82-4.56 (m, 1H), 4.37-3.96 (m, 3H), 3.55 (d, J=5.2 Hz, 1H), 3.27-3.11 (m, 1H), 2.97-2.66 (m, 5H), 2.14-1.71 (m, 13H), 1.69-1.55 (m, 3H), 1.47 (s, 9H), 1.30-1.00 (m, 5H).

### Step 3: Synthesis of Intermediate 1-6.

To a solution of Intermediate 1-4 (150 mg, 329.20 μmol, 1 eq) and Intermediate 1-5 (95.90 mg, 329.20 μmol, 1 eq) in DMF (1.5 mL) was added HOAt (44.81 mg, 329.20 μmol, 46.05 μL, 1 eq), EDCI (189.32 mg, 987.59 μmol, 3 eq) and NMM (166.49 mg, 1.65 mmol, 180.96 μL, 5 eq). The mixture was stirred at 25° C. for 0.5 hr. The reaction mixture was diluted with H₂O (5 mL) and extracted with EA (5 mL*3). The combined organic layers were washed with brine (5 mL*3), dried over Na₂SO₄, filtered and concentrated under reduced pressure to give a residue. The crude product was used in the next step without further purification. Intermediate 1-6 (180 mg, 243.30 μmol, 73.91% yield, 98.532% purity) was obtained as a pale yellow solid. LCMS (Method D): Retention time: 0.471 min, [M+H]⁺=729.5. SFC: Retention time: 0.982, 1.096, 1.290, 1.465 min. ¹H NMR (400 MHz, METHANOL-d4) δ=7.63-7.48 (m, 2H), 7.31-7.18 (m, 1H), 4.96 (d, J=6.0 Hz, 1H), 4.68-4.51 (m, 2H), 4.46-4.32 (m, 2H), 4.25-4.11 (m, 2H), 3.49-3.37 (m, 1H), 3.26-3.14 (m, 2H), 3.09-3.00 (m, 2H), 2.97-2.90 (m, 2H), 2.83-2.69 (m, 1H), 2.21-1.94 (m, 6H), 1.92-1.73 (m, 8H), 1.72-1.53 (m, 6H), 1.47 (s, 9H), 1.37-1.22 (m, 3H), 1.20-1.07 (m, 2H), 0.93-0.75 (m, 4H).

### Step 4: Synthesis of Intermediate 1-7.

To a solution of Intermediate 1-6 (140 mg, 192.06 μmol, 1 eq) in DCM (1 mL) was added TFA (0.2 mL). The mixture was stirred at 25° C. for 0.5 hr. The reaction mixture was concentrated under reduced pressure to give a residue, which was washed with PE. The crude product was used in the next step without further purification. Intermediate 1-7 (160 mg, crude, TFA salt) was obtained as a yellow oil. LCMS (Method D): Retention time: 0.355 min, [M+H]⁺=629.4.

### Step 5: Synthesis of I-694.

To a solution of Intermediate 1-7 (100 mg, 134.61 μmol, 1 eq, TFA salt) and Intermediate 1-8 (59.62 mg, 134.61 μmol, 1 eq) in ACN (1 mL) was added DIEA (52.19 mg, 403.84 μmol, 70.34 μL, 3 eq). The mixture was stirred at 40° C. for 2 hr. The reaction mixture was diluted with H₂O (5 mL) and extracted with EA (5 mL*3). The combined organic layers were washed with brine (5 mL*3), dried over Na₂SO₄, filtered and concentrated under reduced pressure to give a residue. The crude product was purified by reversed-phase chromatography (0.1% TFA condition) and the eluent was concentrated and lyophilized to give the desired product. I-694 (58.84 mg, 51.04 μmol, 37.92% yield, 99.696% purity, TFA salt) was obtained as a white solid. LCMS (Method D): Retention time: 0.391 min, [M+H]⁺=1035.8. SFC: Retention time: 5.815, 6.325, 7.965, 9.092 min. ¹H NMR (400 MHz, METHANOL-d4) δ=8.37 (d, J=7.8 Hz, 1H), 7.99-7.93 (m, 1H), 7.91-7.80 (m, 2H), 7.60-7.45 (m, 3H), 7.41-7.33 (m, 1H), 7.30-7.21 (m, 1H), 7.21-7.14 (m, 1H), 4.99-4.92 (m, 1H), 4.73-4.52 (m, 2H), 4.48-4.23 (m, 6H), 3.86-3.70 (m, 4H), 3.64-3.46 (m, 3H), 3.45-3.33 (m, 4H), 3.27-3.16 (m, 4H), 3.11-2.67 (m, 3H), 2.33-2.08 (m, 5H), 2.07-1.98 (m, 2H), 1.98-1.59 (m, 12H), 1.41-1.20 (m, 4H), 1.19-1.07 (m, 2H), 0.92-0.77 (m, 4H). ¹⁹FNMR (376 MHz, METHANOL-d4) δ=−77.224, −120.700, −121.758.

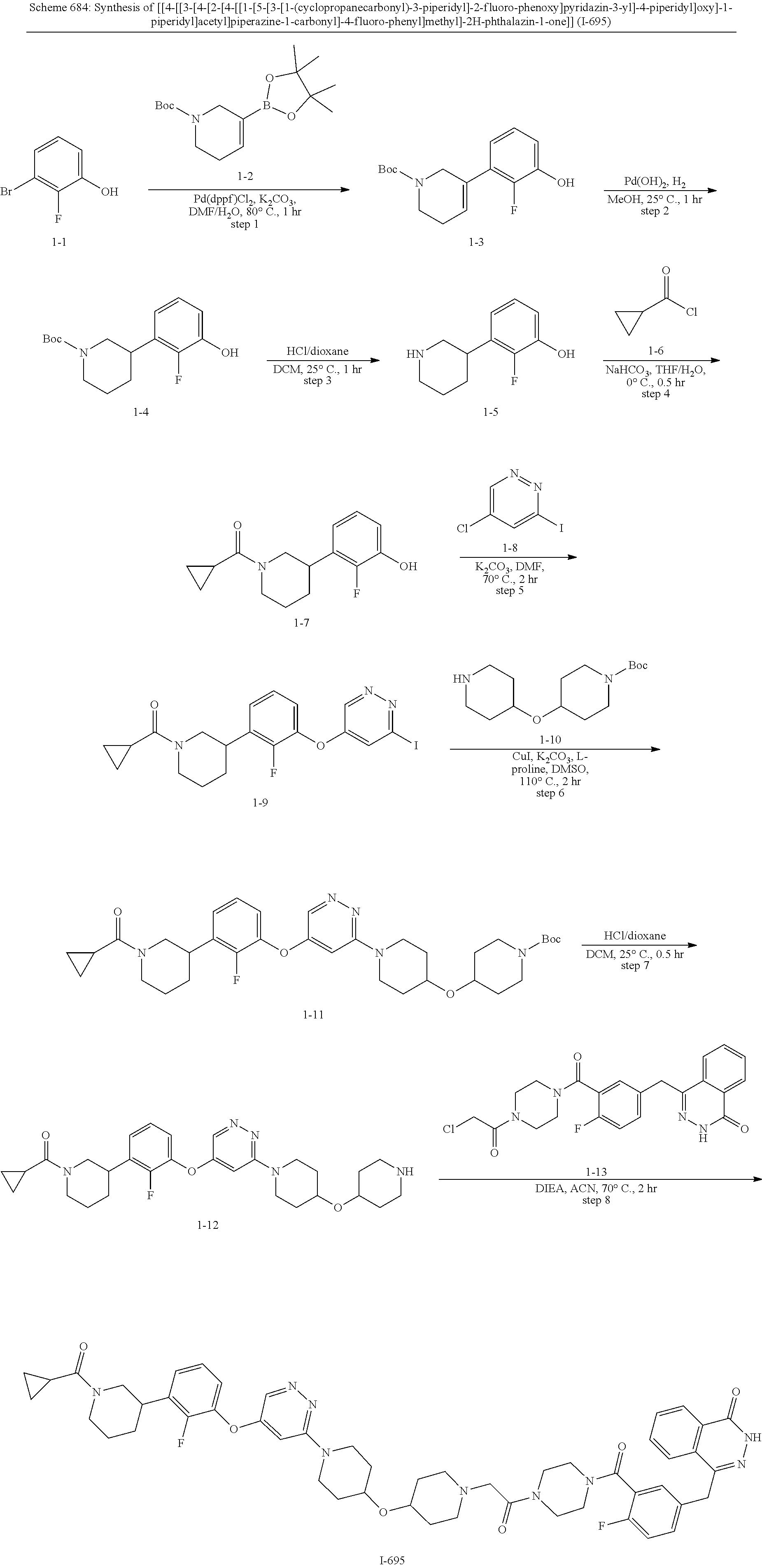

### Step 1: Synthesis of Intermediate 1-3.

A mixture of Intermediate 1-1 (1 g, 5.24 mmol, 1 eq), Intermediate 1-2 (1.94 g, 6.28 mmol, 1.2 eq), Pd(dtbpf)Cl₂ (341.23 mg, 523.57 gmol, 0.1 eq), and K₂CO₃ (2.17 g, 15.71 mmol, 3 eq) in DMF (10 mL) and H₂O (2 mL) was degassed and purged with N₂ 3 times, and then the mixture was stirred at 80° C. for 1 hr under N₂ atmosphere. The mixture was washed with water (10 mL) and extracted with EA (15 mL*3). The combined organic layers were dried over sodium sulfate, filtered and concentrated under reduced pressure to give a residue. The residue was purified by flash silica gel chromatography (ISCO; 40 g SepaFlash Silica Flash Column, Eluent of 0˜20% EA/PE@60 mL/min) and then the eluent was concentrated in vacuo to give product. Intermediate 1-3 (1.4 g, 4.63 mmol, 88.42% yield, 97% purity) was obtained as a yellow solid. LCMS (Method D): Rt=0.439 min, [M-tBu+H]⁺=238.1. ¹H NMR (400 MHz, CHLOROFORM-d) δ=7.02-6.89 (m, 2H), 6.77-6.75 (m, 1H), 6.07 (s, 1H), 4.20 (s, 2H), 3.58-3.57 (m, 2H), 2.32 (d, J=2.8 Hz, 2H), 1.49 (s, 9H). ¹⁹F NMR (376 MHz, CHLOROFORM-d) δ=−143.814.

### Step 2: Synthesis of Intermediate 1-4.

To a solution of Intermediate 1-3 (1.4 g, 4.77 mmol, 1 eq) in MeOH (15 mL) was added Pd(OH)₂ (300 mg, 427.24 μmol, 20% purity, 8.95e−2 eq) under N₂ atmosphere. The suspension was degassed and purged with H₂ 3 times. The mixture was stirred under H₂ (15 Psi) at 25° C. for 1 hr. The resulting mixture was filtered, and the filtrate was concentrated under reduced pressure. The crude product was used in the next step without further purification. Intermediate 1-4 (1.2 g, 4.06 mmol, 85.13% yield) was obtained as a white solid. LCMS (Method D): Rt=0.435 min, [M-tBu+H]⁺=240.1. ¹H NMR (400 MHz, CHLOROFORM-d) δ=7.00-6.94 (m, 1H), 6.93-6.85 (m, 1H), 6.73-6.72 (m, 1H), 4.18 (s, 2H), 2.98-2.97 (m, 1H), 2.85-2.72 (m, 2H), 1.99 (d, J=11.2 Hz, 1H), 1.77 (d, J=12.0 Hz, 1H), 1.70-1.61 (m, 2H), 1.48 (s, 9H). ¹⁹F NMR (376 MHz, CHLOROFORM-d) δ=−145.653, −146.332.

### Step 3: Synthesis of Intermediate 1-5

To a solution of Intermediate 1-4 (1.6 g, 5.42 mmol, 1 eq) in DCM (15 mL) was added HCl/dioxane (2 M, 15 mL). The mixture was stirred at 25° C. for 0.5 hr. The reaction mixture was concentrated in vacuo. The crude product was used in the next step without further purification. Intermediate 1-5 (1.2 g, crude, HCl salt) was obtained as a white solid. LCMS (Method A): Rt=0.170 min, [M+H]⁺=196.0. ¹H NMR (400 MHz, METHANOL-d4) δ=7.01-6.95 (m, 1H), 6.85-6.84 (m, 1H), 6.76-6.74 (m, 1H), 3.46-3.39 (m, 2H), 3.38-3.32 (m, 1H), 3.15-3.08 (m, 1H), 3.08-3.00 (m, 1H), 2.10-1.99 (m, 2H), 1.94-1.84 (m, 2H). ¹⁹F NMR (376 MHz, METHANOL-d4) δ=−145.360.

### Step 4: Synthesis of Intermediate 1-7

To a solution of Intermediate 1-5 (1.2 g, 5.18 mmol, 1 eq, HCl salt) and NaHCO₃ (1.31 g, 15.54 mmol, 604.57 μL, 3 eq) in H₂O (6 mL) was added a solution of Intermediate 1-6 (649.69 mg, 6.22 mmol, 563.97 μL, 1.2 eq) in THF (6 mL) at 0° C. The mixture was stirred at 25° C. for 0.5 hr. The mixture was diluted with DCM, washed with water (10 mL) and extracted with DCM (10 mL*2). The combined organic layers were dried over sodium sulfate, filtered and concentrated under reduced pressure to give a residue. The residue was purified by flash silica gel chromatography (ISCO; 40 g SepaFlash Silica Flash Column, Eluent of 0˜50% EA/PE @60 mL/min) and then the eluent was concentrated in vacuo to give the product. Intermediate 1-7 (630 mg, 2.37 mmol, 45.73% yield, 99% purity) was obtained as a yellow gum. LCMS (Method D): Rt=0.357 min, [M+H]⁺=264.1. ¹H NMR (400 MHz, DMSO-d6) δ=9.72 (d, J=6.8 Hz, 1H), 6.93 (s, 1H), 6.86-6.70 (m, 2H), 4.41 (d, J=11.2 Hz, 1H), 4.33-4.19 (m, 1H), 3.21-3.07 (m, 1H), 2.96-2.76 (m, 1H), 2.69-2.58 (m, 1H), 1.99-1.83 (m, 2H), 1.82-1.67 (m, 2H), 1.58-1.38 (m, 1H), 0.78-0.65 (m, 4H). ¹⁹F NMR (376 MHz, DMSO-d6) δ=−142.834.

### Step 5: Synthesis of Intermediate 1-9

To a solution of Intermediate 1-7 (262.84 mg, 998.22 μmol, 1 eq) and Intermediate 1-8 (240 mg, 998.22 μmol, 1 eq) in DMF (2.5 mL) was added K₂CO₃ (413.88 mg, 2.99 mmol, 3 eq). The mixture was stirred at 70° C. for 2 hr. The mixture was diluted with EA, washed with water (1 mL) and extracted with EA (2 mL*3). The combined organic layers were dried over sodium sulfate, filtered and concentrated under reduced pressure to give a residue. The residue was purified by flash silica gel chromatography (ISCO; 80 g SepaFlash Silica Flash Column, Eluent of 0˜100% MeOH/EA @100 mL/min) and then the eluent was concentrated in vacuo to give product. Intermediate 1-9 (300 mg, 642.02 μmol, 64.32% yield, 100% purity) was obtained as a white solid. LCMS (Method D): Rt=0.425 min, [M+H]⁺=468.1.

### Step 6: Synthesis of Intermediate 1-11

A mixture of Intermediate 1-9 (260 mg, 556.42 μmol, 1 eq), Intermediate 1-10 (158.24 mg, 556.42 μmol, 1 eq), CuI (10.60 mg, 55.64 μmol, 0.1 eq), K₂CO₃ (230.70 mg, 1.67 mmol, 3 eq) and L-proline (12.81 mg, 111.28 μmol, 0.2 eq) in DMSO (3 mL) was degassed and purged with N₂ 3 times, and then the mixture was stirred at 110° C. for 1 hr under N₂ atmosphere. The mixture was washed with aqueous NaCl (3 mL) and extracted with EA (3 mL*3). The combined organic layers were dried over sodium sulfate, filtered and concentrated under reduced pressure to give a residue. The residue was purified by reversed phase chromatography (0.1% FA condition), and the eluent was concentrated under reduced pressure to remove ACN and lyophilized to give product. Intermediate 1-11 (90 mg, 125.53 μmol, 22.56% yield, 87% purity) was obtained as a white solid. LCMS (Method D): Rt=0.405 min, [M+H]⁺=624.5.

### Step 7: Synthesis of Intermediate 1-12

To a solution of Intermediate 1-11 (90 mg, 144.29 μmol, 1 eq) in DCM (1 mL) was added HCl/dioxane (2 M, 1 mL). The mixture was stirred at 25° C. for 0.5 hr. The reaction mixture was concentrated in vacuo. The crude product was used in the next step without further purification. Intermediate 1-12 (90 mg, crude, HCl salt) was obtained as a yellow oil. LCMS (Method D): Rt=0.293 min, [M+H]⁺=524.3.

### Step 8: Synthesis of I-695.

To a solution of Intermediate 1-12 (90 mg, 160.69 μmol, 1 eq, HCl salt) and Intermediate 1-13 (71.16 mg, 160.69 μmol, 1 eq) in ACN (1 mL) was added DIEA (62.30 mg, 482.06 μmol, 83.96 μL, 3 eq). The mixture was stirred at 70° C. for 1 hr. The mixture was washed with water (3 mL) and extracted with EA (5 mL*3). The combined organic layers were dried over sodium sulfate, filtered and concentrated under reduced pressure to give a residue. The residue was purified by prep-HPLC (column: CD03-Welch Xtimate C18 150*25*5 um; mobile phase: [water(FA)-ACN]; gradient: 15%-45% B over 10 min) and the eluent was concentrated under reduced pressure to remove ACN and lyophilized to give product. I-695 (23.78 mg, 23.67 μmol, 14.73% yield, 97.171% purity, FA salt) was obtained as a white solid. LCMS (Method D): Rt=0.345 min, [M+H]⁺=930.5. SFC: Rt=4.518 min, 6.603 min. ¹H NMR (400 MHz, CHLOROFORM-d) δ=10.43-10.11 (m, 1H), 8.49-8.43 (m, 1H), 8.37 (d, J=13.6 Hz, 1H), 8.21-8.15 (m, 1H), 7.81-7.70 (m, 3H), 7.36-7.31 (m, 2H), 7.17 (d, J=19.2 Hz, 2H), 7.11-7.01 (m, 2H), 6.36-6.16 (m, 1H), 4.79-4.60 (m, 1H), 4.36-4.26 (m, 3H), 3.95 (s, 2H), 3.84-3.77 (m, 1H), 3.69 (d, J=3.6 Hz, 2H), 3.59 (d, J=14.0 Hz, 2H), 3.51 (d, J=1.6 Hz, 1H), 3.31 (d, J=19.2 Hz, 5H), 3.18-3.12 (m, 1H), 3.11-2.95 (m, 1H), 2.85 (d, J=1.2 Hz, 2H), 2.80-2.55 (m, 1H), 2.37-2.36 (m, 2H), 2.13-2.01 (m, 3H), 1.91 (s, 8H), 1.79 (s, 1H), 1.68-1.59 (m, 4H), 1.01 (d, J=2.8 Hz, 2H), 0.78 (d, J=6.4 Hz, 2H). ¹⁹F NMR (376 MHz, CHLOROFORM-d) δ=−117.58, −134.42.

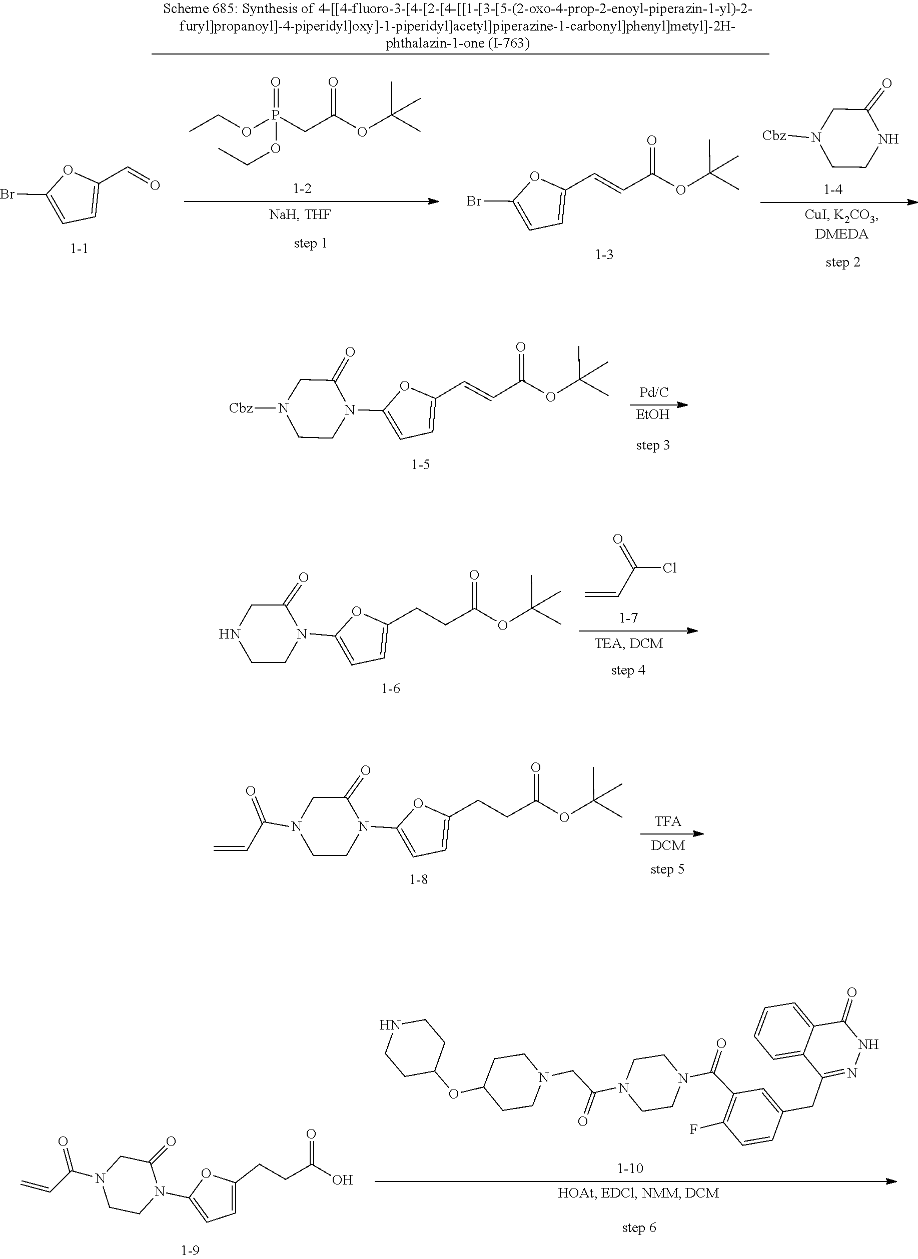

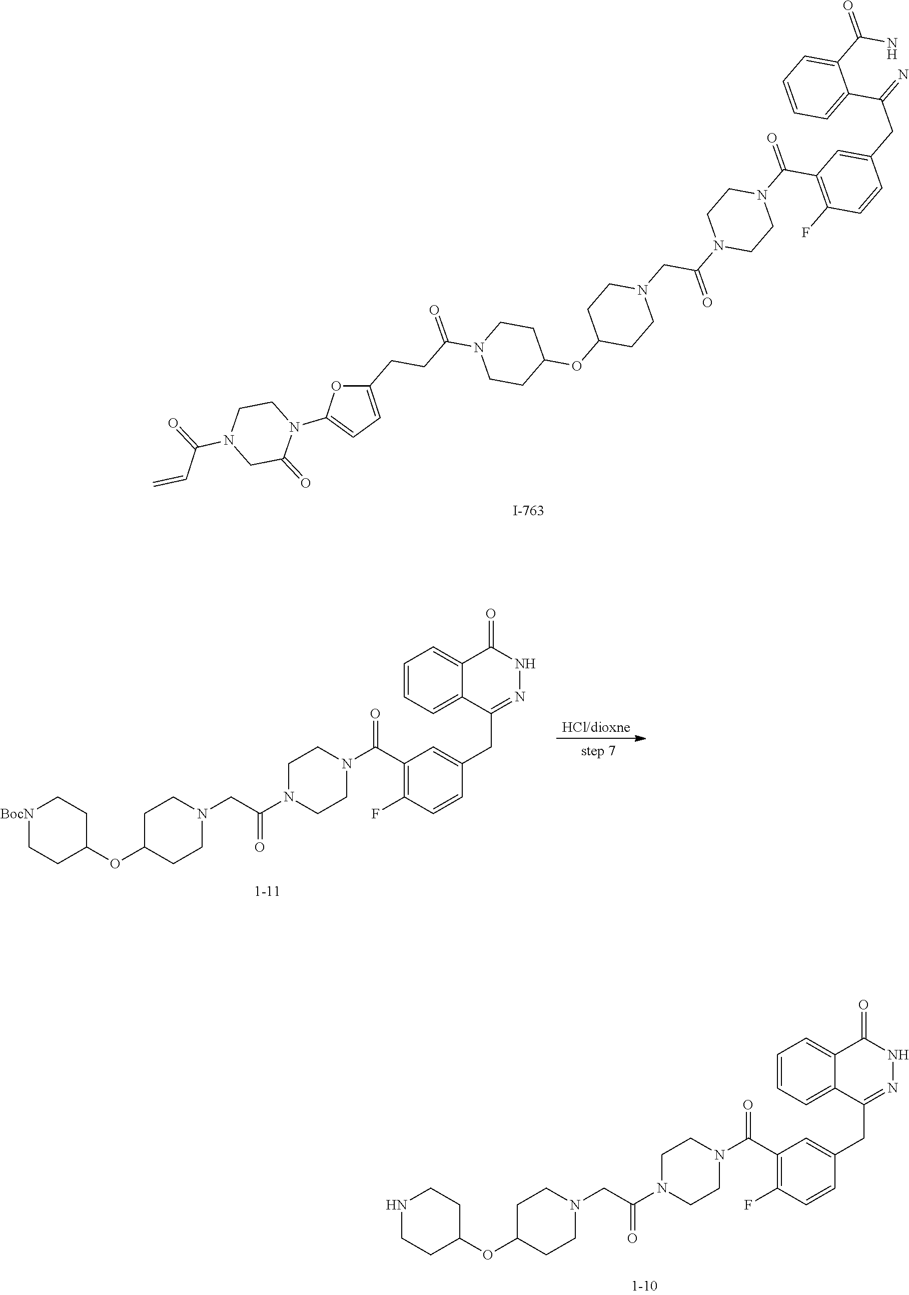 [SMILES: C=CC(=O)N1CCN(c2ccc(CCC(=O)N3CCC(OC4CCN(CC(=O)N5CCN(C(=O)c6cc(CC(=N)c7ccccc7C(N)=O)ccc6F)CC5)CC4)CC3)o2)C(=O)C1 ; CC(C)(C)OC(=O)N1CCC(OC2CCN(CC(=O)N3CCN(C(=O)c4cc(Cc5n[nH]c(=O)c6ccccc56)ccc4F)CC3)CC2)CC1 ; O=C(CN1CCC(OC2CCNCC2)CC1)N1CCN(C(=O)c2cc(Cc3n[nH]c(=O)c4ccccc34)ccc2F)CC1]

### Step 1: Synthesis of Intermediate 1-3

To a solution of NaH (5.94 g, 148.59 mmol, 60% purity, 1.3 eq) in THF (60 mL) was added dropwise a solution of Intermediate 1-2 (34.60 g, 137.16 mmol, 1.2 eq) dissolved in THF (60 mL) at 0° C. After addition, the mixture was stirred at this temperature for 30 min, and then Intermediate 1-1 (20 g, 114.30 mmol, 1 eq) in THF (60 mL) was added dropwise at 0° C. The resulting mixture was stirred at 0° C. for 1 hr. The reaction mixture was poured into aq. NH₄Cl (200 mL) and extracted with EA (200 mL*3), the organic layer was washed with brine (200 mL) and dried over Na₂SO₄. The organic layer was concentrated to give the crude product. The crude product was purified by flash silica gel chromatography (SiO₂, PE:EA=10:1 to 5:1) to give Intermediate 1-3 (30 g, 109.84 mmol, 96.10% yield) as a colorless oil. The colorless oil was used for the next step without further purification.

### Step 2: Synthesis of Intermediate 1-5

To a solution of Intermediate 1-3 (12 g, 43.94 mmol, 1 eq), Intermediate 1-4 (10.29 g, 43.94 mmol, 1 eq), K₂CO₃ (18.22 g, 131.81 mmol, 3 eq), and DMEDA (1.16 g, 13.18 mmol, 1.42 mL, 0.3 eq) in dioxane (120 mL) was added CuI (836.77 mg, 4.39 mmol, 0.1 eq). Then the mixture was stirred at 100° C. for 16 hr under N₂. The reaction mixture was poured into water (200 mL) and extracted with EA (100 mL*2), the organic layer was washed with brine (100 mL) and dried over Na₂SO₄. The organic layer was concentrated to give the crude product. The crude product was purified by flash silica gel chromatography (SiO₂, PE:EA=10:1 to 3:1) to give Intermediate 1-5 (8 g, 18.59 mmol, 42.31% yield, 99.1% purity) as a colorless oil. LCMS (Method E): Rt=0.626 min, [M+H]⁺=427.2.

### Step 3: Synthesis of Intermediate 1-6

To a solution of Intermediate 1-5 (8 g, 18.76 mmol, 1 eq) in EtOH (80 mL) was added Pd/C (1.6 g, 10% purity). Then the mixture was stirred at 25° C. for 1 hr under H₂ (15 psi). The reaction mixture was filtered to give the filtrate, which was concentrated under vacuum to give the crude product as colorless oil. Intermediate 1-6 (5 g, 16.48 mmol, 87.84% yield, 97.0% purity) was obtained as colorless oil. LCMS (Method E): Rt=0.398 min, [M+H-tBu]⁺=239.1.

### Step 4: Synthesis of Intermediate 1-8

To a solution of Intermediate 1-6 (4.9 g, 16.65 mmol, 1 eq) and TEA (3.37 g, 33.29 mmol, 4.63 mL, 2 eq) in DCM (50 mL) was added Intermediate 1-7 (2.26 g, 24.97 mmol, 2.04 mL, 1.5 eq) at 0° C. Then the mixture was stirred at 25° C. for 1 hr. The reaction mixture was poured into water (100 mL) and extracted with DCM (100 mL*2), the organic layer was washed with brine (100 mL), dried over Na₂SO₄, and concentrated to give the crude product. The crude product was purified by flash silica gel chromatography (SiO₂, PE:EA=10:1 to 1:1) to give Intermediate 1-8 (3.5 g, 9.81 mmol, 58.90% yield, 97.6% purity) as a colorless oil. LCMS (Method E): Rt=0.475 min, [M+H-tBu]⁺*=293.0.

### Step 5: Synthesis of Intermediate 1-9

To a solution of Intermediate 1-8 (3.5 g, 10.05 mmol, 1 eq) in DCM (40 mL) was added TFA (10 mL). Then the mixture was stirred at 25° C. for 1 hr. sat. Na₂CO₃ was added to adjust the pH to 8-9, and the mixture was extracted with ethyl acetate (100 mL*2). sat. citric acid was added to adjust the pH to 3-4, and the mixture was extracted with CHCl₃/i-PrOH (3/1) (100 mL*3). The organic layers were dried over Na₂SO₄, filtered and concentrated under reduced pressure to give Intermediate 1-9 (2.5 g, 8.36 mmol, 83.18% yield, 97.7% purity) as a yellow solid. LCMS (Method E): Rt=0.354 min, [M+H]⁺=292.9. ¹H NMR (400 MHz, DMSO-d₆) δ=12.27 (br s, 1H), 7.05-6.67 (m, 1H), 6.35-6.04 (m, 3H), 5.80 (br d, J=10.0 Hz, 1H), 4.50-4.26 (m, 2H), 4.03-3.77 (m, 4H), 2.84 (m, 2H), 2.60-2.56 (m, 2H).

### Step 6: Synthesis of I-763

To a solution of Intermediate 1-9 (79.23 mg, 271.07 μmol, 1 eq), EDCI (103.93 mg, 542.14 μmol, 2 eq), HOAt (18.45 mg, 135.53 μmol, 18.96 μL, 0.5 eq), and NMM (137.09 mg, 1.36 mmol, 149.01 μL, 5 eq) in DCM (2 mL) was added Intermediate 1-10 (170 mg, 271.07 μmol, 1 eq, HCl salt). The mixture was stirred at 25° C. for 1 hr. The reaction mixture was filtered to give the filtrate. The filtrate was purified by prep-HPLC (column: Phenomenex luna C18 150*25 mm*10 um; mobile phase: [water (FA)-ACN]; gradient: 5%-35% B over 15 min) to give I-763 (56.04 mg, 60.57 μmol, 22.34% yield, 98.46% purity, FA salt) as a yellow solid. LCMS: Rt=0.415 min, [M+H]⁺=865.4. ¹H NMR (400 MHz, DMSO-d₆) δ=12.60 (s, 1H), 8.27 (d, J=8.0 Hz, 1H), 8.17 (s, 1H), 8.02-7.94 (m, 1H), 7.93-7.81 (m, 2H), 7.52-7.35 (m, 2H), 7.25-7.23 (m, 1H), 6.92-6.74 (m, 1H), 6.23-6.12 (m, 3H), 5.77 (br d, J=9.2 Hz, 1H), 4.48-4.37 (m, 1H), 4.34 (s, 2H), 4.27 (s, 1H), 4.01-3.72 (m, 6H), 3.71-3.36 (m, 11H), 3.14-2.98 (m, 3H), 2.81-2.75 (m, 2H), 2.71-2.60 (m, 4H), 2.21-2.07 (m, 2H), 1.76 (br d, J=12.0 Hz, 4H), 1.47-1.24 (m, 4H). ¹⁹F NMR (377 MHz, DMSO-d₆) δ=−119.777.

### Step 7: Synthesis of Intermediate 1-10

A solution of Intermediate 1-11 (200 mg, 289.52 μmol, 1 eq) in HCl/dioxane (2 mL) was stirred at 25° C. for 1 hr. The reaction mixture was concentrated to give the crude product. The crude product was triturated with EA (5 mL) at 25° C. for 30 min to give Intermediate 1-10 (180 mg, 264.63 μmol, 91.40% yield, 92.2% purity, HCl salt) as an off-white solid. LCMS (Method E): Rt=0.371 min, [M+H]⁺=591.3.

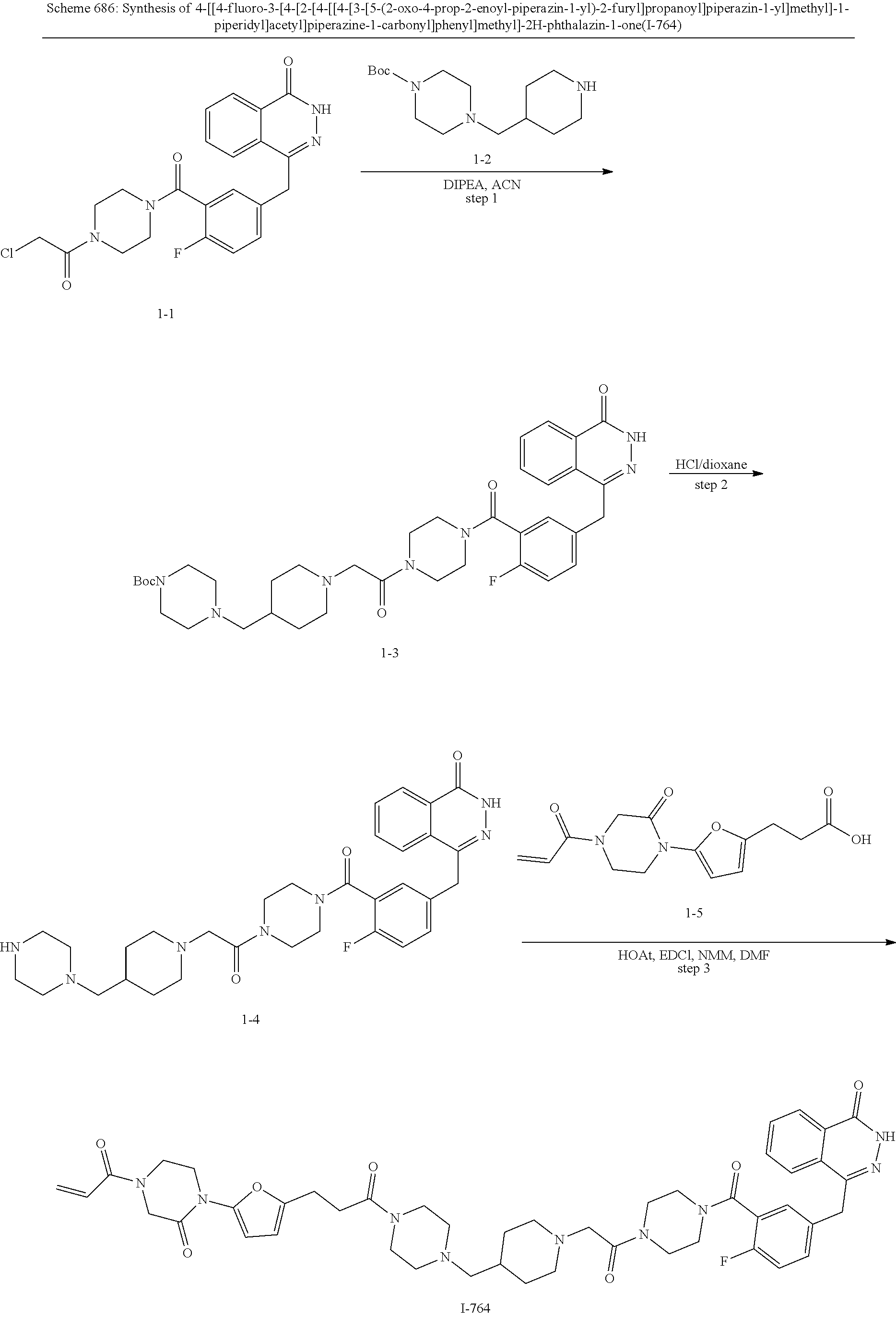

### Step 1: Synthesis of Intermediate 1-3

To a solution of intermediate 1-1 (4 g, 9.03 mmol, 1 eq) and intermediate 1-2 (2.56 g, 9.03 mmol, 1 eq) in DMA (40 mL) was added KI (149.93 mg, 903.20 μmol, 0.1 eq) and K₂CO₃ (4.99 g, 36.13 mmol, 4 eq). The mixture was stirred at 70° C. for 1 hr. The crude was combined with another lot of material for purification. The residue was purified by flash silica gel chromatography (ISCO; 80 g SepaFlash Silica Flash Column, Eluent of 0˜50% Ethyl acetate/Petroleum ether gradient @80 mL/min). Intermediate 1-3 (7 g, 9.34 mmol, 82.69% yield, 92% purity) was obtained as a yellow solid. LCMS (Method E): Rt=0.394 min, M+H=690.3. ¹H NMR (400 MHz, METHANOL-d4) δ=8.36 (d, J=7.6 Hz, 1H), 7.95 (d, J=7.2 Hz, 1H), 7.90-7.79 (m, 2H), 7.56-7.43 (m, 1H), 7.41-7.33 (m, 1H), 7.19-7.13 (m, 1H), 4.38 (s, 2H), 3.84-3.64 (m, 4H), 3.61-3.49 (m, 2H), 3.41 (br s, 4H), 3.35 (s, 2H), 3.26-3.16 (m, 2H), 2.92-2.83 (m, 2H), 2.35 (d, J=4.4 Hz, 4H), 2.22-2.17 (m, 2H), 2.08-2.01 (m, 2H), 1.81-1.68 (m, 2H), 1.61-1.51 (m, 1H), 1.45 (s, 9H), 1.33-1.13 (m, 2H).

### Step 2: Synthesis of Intermediate 1-4

A solution of intermediate 1-3 (300 mg, 434.90 μmol, 1 eq) in HCl/dioxane (3 mL) was stirred at 25° C. for 1 hr. The reaction mixture was concentrated under reduced pressure to give product. Intermediate 1-4 (220 mg, 373.07 μmol, 85.78% yield) was obtained as a white solid. LCMS (Method E): Rt=0.360 min, M+H=590.3. ¹H NMR (400 MHz, METHANOL-d4) δ=8.37 (d, J=7.5 Hz, 1H), 7.99-7.93 (m, 1H), 7.91-7.81 (m, 2H), 7.50 (br s, 1H), 7.38 (br t, J=6.4 Hz, 1H), 7.17 (t, J=9.0 Hz, 1H), 5.49 (s, 2H), 4.85-4.83 (m, 1H), 4.44-4.32 (m, 3H), 4.29 (s, 1H), 3.88-3.79 (m, 2H), 3.75 (br s, 5H), 3.60-3.38 (m, 7H), 3.35 (s, 2H), 3.25-3.11 (m, 2H), 3.09-2.96 (m, 1H), 2.37 (br s, 1H), 2.30-2.20 (m, 2H), 2.12 (br s, 1H), 1.98 (br s, 1H), 1.73 (q, J=12.3 Hz, 2H).

### Step 3: Synthesis of I-764

To a solution of intermediate 1-4 (120 mg, 203.49 μmol, 1 eq) and intermediate 1-5 (59.48 mg, 203.49 μmol, 1 eq) in DMF (2 mL) was added EDCI (78.02 mg, 406.99 μmol, 2 eq), NMM (102.91 mg, 1.02 mmol, 111.86 μL, 5 eq) and HOAt (27.70 mg, 203.49 μmol, 28.47 μL, 1 eq). The mixture was stirred at 25° C. for 1 hr. The reaction mixture was extracted with EtOAc (3 mL*3). The combined organic layers were washed with brine (5 mL*2), dried over Na₂SO₄, filtered and concentrated under reduced pressure to give a residue The residue was purified by prep-HPLC (column: Phenomenex luna C18 150*25 mm*10 um; mobile phase: [water(FA)-ACN]; gradient: 11%-30% B over 10 min). I-764 (61 mg, 67.03 μmol, 32.94% yield, FA salt) was obtained as a white solid. LCMS (Method E): Rt=0.384 min, M+H=864.5. ¹H NMR (400 MHz, DMSO-d6) δ=12.59 (s, 1H), 8.26 (d, J=7.6 Hz, 1H), 8.16 (s, 2H), 7.99-7.94 (m, 1H), 7.92-7.78 (m, 2H), 7.48-7.33 (m, 2H), 7.29-7.18 (m, 1H), 6.89-6.71 (m, 1H), 6.24-6.08 (m, 3H), 5.80-5.68 (m, 1H), 4.44-4.22 (m, 4H), 3.98-3.72 (m, 5H), 3.68-3.55 (m, 4H), 3.49 (br d, J=18.0 Hz, 3H), 3.38 (br d, J=4.4 Hz, 1H), 3.23-3.09 (m, 5H), 2.85-2.71 (m, 4H), 2.65-2.57 (m, 2H), 2.33-2.21 (m, 4H), 2.14-1.93 (m, 4H), 1.72-1.57 (m, 2H), 1.54-1.37 (m, 1H), 1.17-0.96 (m, 2H).

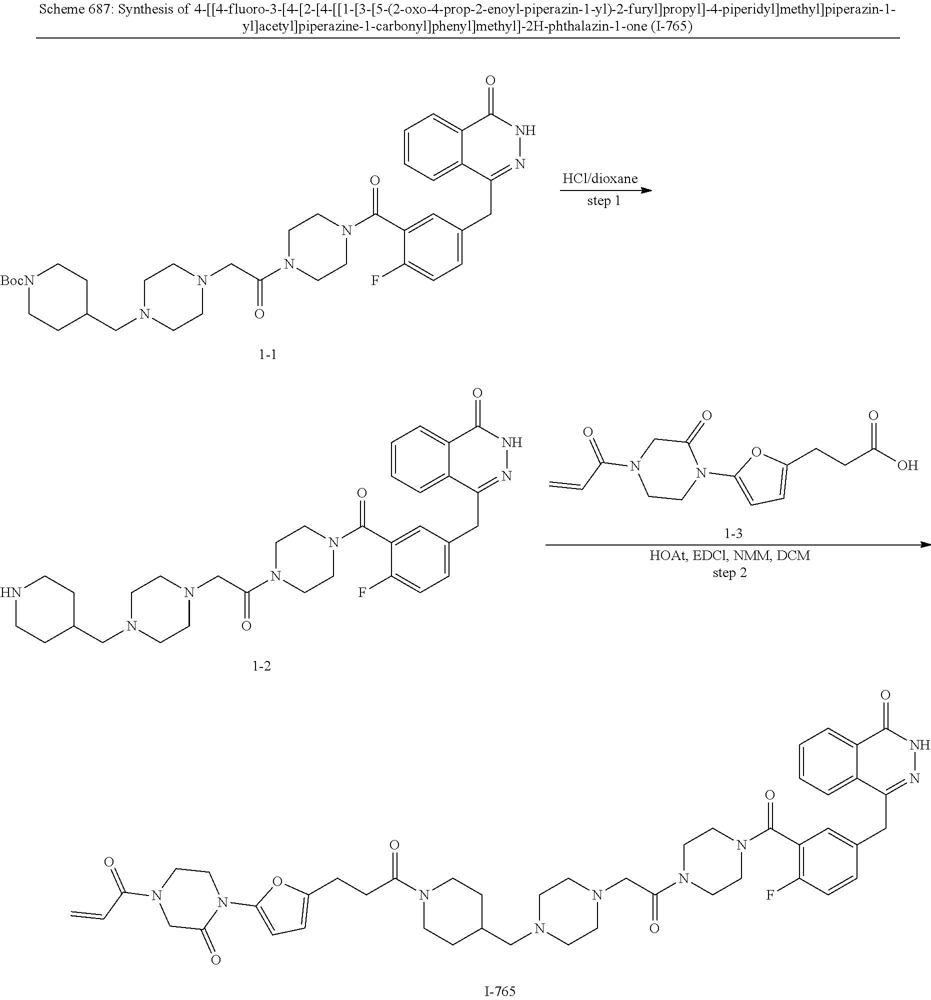

### Step 1: Synthesis of Intermediate 1-2

A solution of Intermediate 1-1 (200 mg, 289.93 μmol, 1 eq) in HCl/dioxane (2 M, 2 mL) was stirred at 25° C. for 1 hr. The reaction mixture was concentrated to give the crude product. The crude product was triturated with EA (5 mL) at 25° C. for 30 min to give Intermediate 1-2 (180 mg, 274.53 μmol, 94.69% yield, 95.5% purity, HCl salt) as an off-white solid. LCMS (Method E): Rt=0.363 min, [M+H]⁺=590.3.

### Step 2: Synthesis of I-765

To a solution of Intermediate 1-3 (79.35 mg, 271.49 μmol, 1 eq), EDCI (104.09 mg, 542.99 μmol, 2 eq), HOAt (18.48 mg, 135.75 μmol, 18.99 μL, 0.5 eq), and NMM (137.30 mg, 1.36 mmol, 149.24 μL, 5 eq) in DCM (2 mL) was added Intermediate 1-2 (170 mg, 271.49 μmol, 1 eq, HCl salt). Then the mixture was stirred at 25° C. for 1 hr. The reaction mixture was filtered to give the filtrate. The filtrate was purified by prep-HPLC (column: Phenomenex luna C18 150*25 mm*10 um; mobile phase: [water (FA)-ACN]; gradient: 5%-35% B over 15 min) to give I-765 (58.27 mg, 63.72 μmol, 23.47% yield, 99.51% purity, FA salt) as a yellow solid. LCMS (Method E): Rt=0.409 min, [M+H]⁺=864.5. ¹H NMR (400 MHz, DMSO-d6) δ=12.60 (s, 1H), 8.27 (d, J=7.6 Hz, 1H), 8.17 (s, 1H), 8.03-7.95 (m, 1H), 7.93-7.81 (m, 2H), 7.58-7.33 (m, 2H), 7.26-7.24 (m, 1H), 6.82 (br d, J=11.2 Hz, 1H), 6.27-6.08 (m, 3H), 5.82-5.67 (m, 1H), 4.45-4.25 (m, 5H), 4.01-3.70 (m, 6H), 3.68-3.34 (m, 9H), 3.10 (br s, 1H), 2.96-2.94 (m, 1H), 2.82-2.75 (m, 2H), 2.65-2.54 (m, 3H), 2.45-2.25 (m, 7H), 2.10-2.08 (m, 2H), 1.68 (br d, J=2.8 Hz, 3H), 1.02-0.79 (m, 2H). ¹⁹F NMR (377 MHz, DMSO-d₆) δ=−119.783.

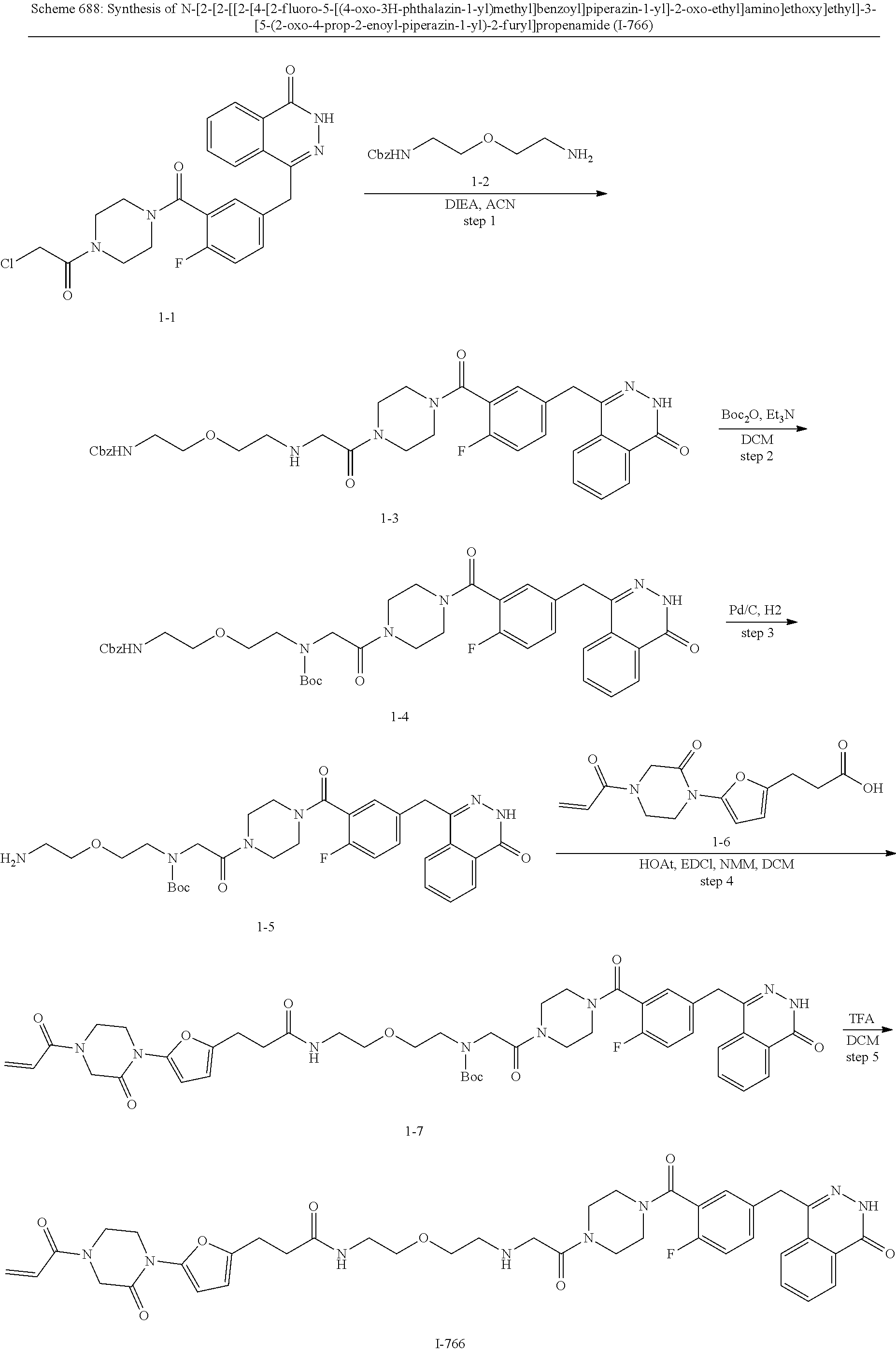

### Step 1: Synthesis of Intermediate 1-3

To a solution of intermediate 1-1 (800 mg, 1.81 mmol, 1 eq) and intermediate 1-2 (860.87 mg, 3.61 mmol, 2 eq) in ACN (8 mL) was added DIEA (466.93 mg, 3.61 mmol, 629.28 μL, 2 eq). The mixture was stirred at 30° C. for 3 hr. The residue was diluted with H₂O (10 mL) and extracted with EtOAc (10 mL*3). The combined organic layers were dried over Na₂SO₄, filtered and concentrated under reduced pressure to give a residue. The residue was purified by flash silica gel chromatography (ISCO; 40 g SepaFlash Silica Flash Column, Eluent of 0-30% DCM:MeOH @60 mL/min). Intermediate 1-3 (615 mg, 925.33 μmol, 51.22% yield, 97% purity) was obtained as a white solid. LCMS (Method E): Rt=0.422 min, M+H=645.2.

### Step 2: Synthesis of Intermediate 1-4

To a solution of intermediate 1-3 (550 mg, 853.12 μmol, 1 eq) in DCM (5.5 mL) was added Boc₂O (372.39 mg, 1.71 mmol, 391.99 μL, 2 eq) and TEA (172.65 mg, 1.71 mmol, 237.49 μL, 2 eq). The mixture was stirred at 25° C. for 1 hr. The residue was diluted with H₂O (10 mL) and extracted with EtOAc (10 mL*3). The combined organic layers were dried over Na₂SO₄, filtered and concentrated under reduced pressure to give a residue. The residue was purified by flash silica gel chromatography (ISCO; 20 g SepaFlash Silica Flash Column, Eluent of 0˜20% DCM/MeOH @40 mL/min). Intermediate 1-4 (560 mg, 684.20 μmol, 80.20% yield, 91% purity) was obtained as a white solid. LCMS (Method E): Rt=0.508 min, M+H=745.5.

### Step 3: Synthesis of Intermediate 1-5

To a solution of intermediate 1-4 (260 mg, 349.08 μmol, 1 eq) in MeOH (5 mL) was added Pd/C (100 mg, 10% purity, 1.00 eq) under N₂ atmosphere. The suspension was degassed and purged with H₂ 3 times. The mixture was stirred under H₂ (50 Psi) at 25° C. for 5 hr. The reaction mixture was washed by MeOH (5 mL*3) and then filtered to give a residue. The residue was used in the next step without further purification. Intermediate 1-5 (170 mg, 278.38 μmol, 79.75% yield) was obtained as an off-white oil. LCMS (Method E): Rt=0.401 min, M+H=611.4.

### Step 4: Synthesis of Intermediate 1-7

To a solution of intermediate 1-5 (70 mg, 114.63 μmol, 1 eq) and intermediate 1-6 (33.50 mg, 114.63 μmol, 1 eq) in DMF (1 mL) was added EDCI (43.95 mg, 229.25 μmol, 2 eq), NMM (57.97 mg, 573.14 μmol, 63.01 μL, 5 eq), and HOAt (15.60 mg, 114.63 μmol, 16.03 μL, 1 eq). The mixture was stirred at 25° C. for 1 hr. The reaction mixture was extracted with EtOAc (3 mL*3). The combined organic layers were washed with brine (5 mL*2), dried over Na₂SO₄, filtered and concentrated under reduced pressure to give a residue. The residue was used in the next step without further purification. Intermediate 1-7 (70 mg, 79.10 μmol, 69.01% yield) was obtained as a white solid. LCMS (Method E): Rt=0.453 min, M+H=885.3.

### Step 5: Synthesis of I-766

To a solution of intermediate 1-7 (70 mg, 79.10 μmol, 1 eq) in DCM (1 mL) was added TFA (1 mL). The mixture was stirred at 25° C. for 1 hr. The reaction mixture was concentrated under reduced pressure to give a residue. The residue was purified by prep-HPLC (FA condition). I-766 (35 mg, 42.13 μmol, 53.26% yield, 100% purity, FA salt) was obtained as a white solid. LCMS (Method E): Rt=0.406 min, M+H=785.3. ¹H NMR (400 MHz, DMSO-d6) δ=12.60 (s, 1H), 8.26 (d, J=7.6 Hz, 1H), 8.16 (s, 1H), 7.96 (d, J=7.6 Hz, 2H), 7.92-7.79 (m, 2H), 7.48-7.42 (m, 1H), 7.38-7.34 (m, 1H), 7.27-7.21 (m, 1H), 6.89-6.72 (m, 1H), 6.24-6.02 (m, 3H), 5.76 (d, J=9.2 Hz, 1H), 4.45-4.23 (m, 4H), 3.99-3.74 (m, 4H), 3.71-3.59 (m, 4H), 3.58-3.49 (m, 4H), 3.46 (s, 1H), 3.40 (d, J=2.8 Hz, 2H), 3.31 (s, 1H), 3.24-3.16 (m, 4H), 2.86-2.74 (m, 4H), 2.44-2.35 (m, 2H). ¹⁹F NMR (376 MHz, DMSO-d6) δ=−73.44 (s, 2F), −119.71 (s, 1F).

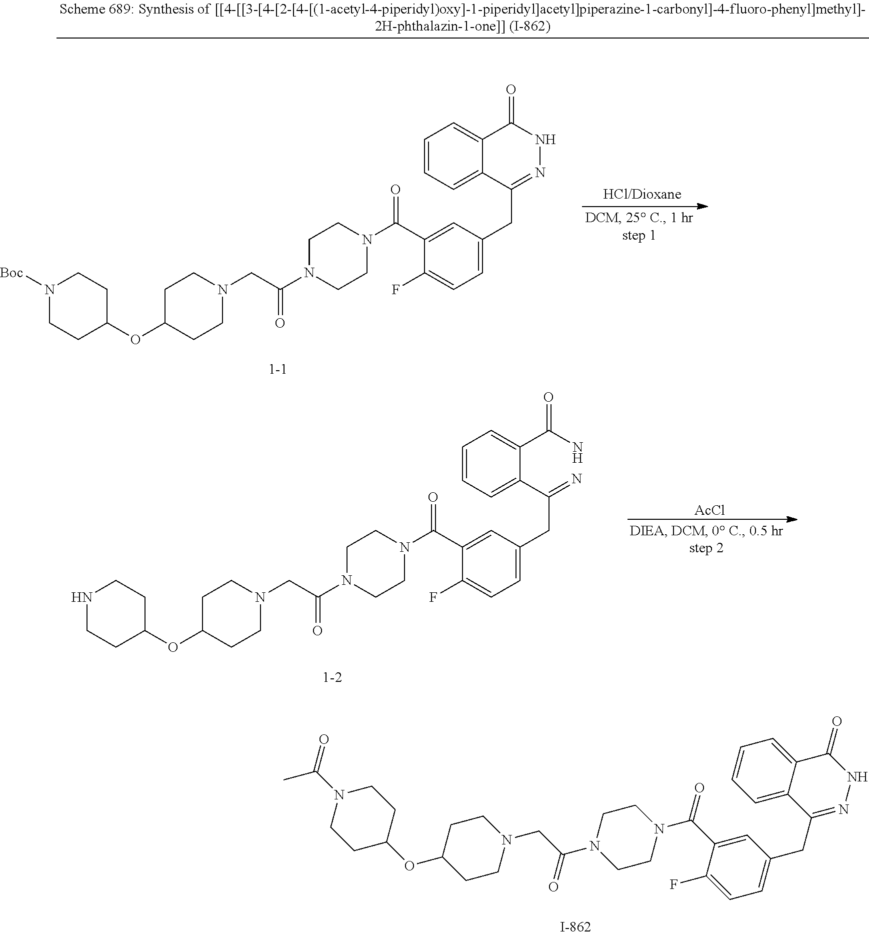

### Step 1: Synthesis of Intermediate 1-2

To a solution of Intermediate 1-1 (100 mg, 144.76 μmol, 1 eq) in DCM (0.7 mL) was added HCl/dioxane (2 M, 0.35 mL, 4.84 eq). The mixture was stirred at 25° C. for 1 hr. The reaction liquid was concentrated under reduced pressure to obtain the crude product. The crude product was used in the next step without further purification. Intermediate 1-2 (90 mg, crude, HCl salt) was obtained as a white solid. LCMS (Method D): Rt=0.241 min, [M+H]⁺=591.3.

### Step 2: Synthesis of I-862

To a solution of Intermediate 1-2 (70 mg, 111.62 μmol, 1 eq, HCl salt) in DCM (0.7 mL) was added DIEA (28.85 mg, 223.23 μmol, 38.88 μL, 2 eq) and acetyl chloride (8.76 mg, 111.62 μmol, 7.94 μL, 1 eq). The mixture was stirred at 0° C. for 0.5 hr. The residue was diluted with H₂O (5 mL) and extracted with EA (5 mL*3), dried over Na₂SO₄, filtered and concentrated under reduced pressure to give a residue. The crude product was purified by reversed-phase HPLC (0.1% TFA condition). The eluent was concentrated in vacuum to remove ACN and lyophilized. I-862 (27.97 mg, 40.59 μmol, 36.37% yield, 98.503% purity, FA salt) was obtained as a white solid. LCMS (Method D): Rt=0.288 min, [M+H]⁺=633.1. ¹H NMR (400 MHz, METHANOL-d4) δ=8.42 (s, 1H), 8.37 (d, J=7.6 Hz, 1H), 7.94 (d, J=4.8 Hz, 1H), 7.91-7.76 (m, 2H), 7.51 (d, J=3.2 Hz, 1H), 7.37 (s, 1H), 7.17 (t, J=8.8 Hz, 1H), 4.39 (s, 2H), 3.94-3.84 (m, 1H), 3.81 (s, 2H), 3.77-3.69 (m, 5H), 3.61 (s, 1H), 3.54 (s, 1H), 3.46 (s, 1H), 3.27-3.19 (m, 1H), 3.17-3.04 (m, 2H), 2.93-2.70 (m, 2H), 2.09 (s, 3H), 2.03 (s, 2H), 1.86-1.72 (m, 3H), 1.62-1.46 (m, 2H), 1.37 (d, J=6.4 Hz, 5H). ¹⁹F NMR (377 MHz, METHANOL-d₄), δ=−120.777.

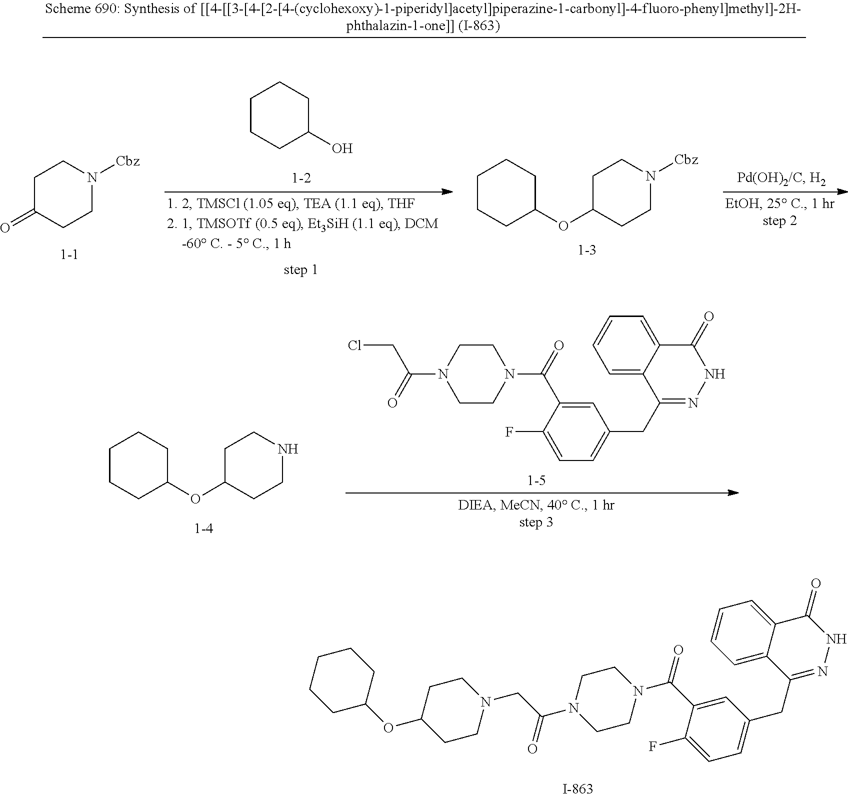 [SMILES: OC1CCCCC1 ; O=C1CCN(C(=O)OCc2ccccc2)CC1 ; O=C(OCc1ccccc1)N1CCC(OC2CCCCC2)CC1 ; C1CCC(OC2CCNCC2)CC1 ; O=C(CCl)N1CCN(C(=O)c2cc(Cc3n[nH]c(=O)c4ccccc34)ccc2F)CC1 ; O=C(CN1CCC(OC2CCCCC2)CC1)N1CCN(C(=O)c2cc(Cc3n[nH]c(=O)c4ccccc34)ccc2F)CC1]

### Step 1: Synthesis of Intermediate 1-3

To a solution of Intermediate 1-2 (429.38 mg, 4.29 mmol, 447.27 μL, 1 eq) in THF (15 mL) was added TEA (477.18 mg, 4.72 mmol, 656.37 μL, 1.1 eq) and the mixture was cooled in an ice bath to give an internal temperature of 5° C. To the solution was added TMSCl (489.04 mg, 4.50 mmol, 571.31 μL, 1.05 eq) dropwise. The resulting slurry was stirred at 5° C. for 30 min. The filtrate was concentrated under reduced pressure. The resulting crude TMS-ether was re-dissolved in DCM (15 mL) and cooled to −60° C. To the cooled solution was added Intermediate 1-1 (1 g, 4.29 mmol, 853.24 μL, 1 eq), Et₃SiH (548.33 mg, 4.72 mmol, 753.20 μL, 1.1 eq), and TMSOTf (476.41 mg, 2.14 mmol, 387.33 μL, 0.5 eq). The reaction mixture was allowed to slowly warm to 0° C. and it was stirred for 30 min. The reaction mixture was concentrated under reduced pressure and treated with 5 ml of HCl (1 M). Then the mixture was extracted with DCM (20 mL*3). The combined organic layers were dried over Na₂SO₄, filtered and concentrated under reduced pressure to give a residue. The residue was purified by flash silica gel chromatography (ISCO; 40 g SepaFlash Silica Flash Column, Eluent of 0˜20% Ethylacetate/Petroleum ether gradient @55 mL/min). Intermediate 1-3 (950 mg, 2.99 mmol, 69.81% yield, 100% purity) was obtained as a colorless oil. LCMS (Method D): Rt=0.505 min, [M+H]⁺=318.0. ¹H NMR (400 MHz, METHANOL-d4) δ=7.40-7.25 (m, 5H), 5.10 (s, 2H), 3.85-3.77 (m, 2H), 3.65-3.63 (m, 1H), 3.45-3.37 (m, 1H), 3.19 (s, 2H), 1.90-1.69 (m, 7H), 1.57-1.50 (m, 1H), 1.49-1.40 (m, 2H), 1.30-1.23 (m, 4H).

### Step 2: Synthesis of Intermediate 1-4

To a solution of Intermediate 1-3 (1.2 g, 3.78 mmol, 1 eq) in EtOH (20 mL) was added Pd(OH)₂/C (20%, 600 mg) under N₂ atmosphere. The suspension was degassed and purged with H₂ 3 times. The mixture was stirred under H₂ (15 Psi) at 25° C. for 1 hr. The reaction mixture was filtered and concentrated under reduced pressure to give a residue. The crude product was used in the next step without further purification. Intermediate 1-4 (660 mg, 3.60 mmol, 95.25% yield) was obtained as a colorless oil. LCMS (Method D): Rt=0.362 min, (M−H)=184.1. ¹H NMR (400 MHz, CHLOROFORM-d) δ=3.49-3.39 (m, 1H), 3.38-3.27 (m, 1H), 3.15-3.01 (m, 2H), 2.65-2.52 (m, 2H), 1.97-1.78 (m, 6H), 1.77-1.66 (m, 2H), 1.56-1.47 (m, 1H), 1.46-1.33 (m, 2H), 1.27-1.18 (m, 4H).

### Step 3: Synthesis of I-863

To a solution of Intermediate 1-4 (50 mg, 272.79 μmol, 1.2 eq) in MeCN (0.5 mL) was added DIEA (88.14 mg, 681.98 μmol, 118.79 μL, 3 eq) and Intermediate 1-5 (100.68 mg, 227.33 μmol, 1 eq). The mixture was stirred at 40° C. for 1 hr. The reaction mixture was filtered and concentrated under reduced pressure to give a residue. The crude product was purified by reversed phase chromatography (0.1% FA condition) followed by lyophilization to give product. I-863 (66.41 mg, 104.46 μmol, 88.00% yield, 100% purity, FA salt) was obtained as a white solid. LCMS (Method D): Rt=0.307 min, [M+H]⁺=590.8. ¹HNMR (400 MHz, METHANOL-d4) δ=8.41 (s, 1H), 8.36 (d, J=7.6 Hz, 1H), 7.98-7.92 (m, 1H), 7.90-7.80 (m, 2H), 7.55-7.46 (m, 1H), 7.41-7.34 (m, 1H), 7.17 (t, J=9.2 Hz, 1H), 4.38 (s, 2H), 4.08-3.90 (m, 2H), 3.84-3.67 (m, 4H), 3.60-3.51 (m, 2H), 3.47-3.32 (m, 4H), 3.29-3.18 (m, 2H), 3.03 (s, 2H), 2.07-1.96 (m, 2H), 1.91-1.71 (m, 6H), 1.59-1.50 (m, 1H), 1.38-1.22 (m, 5H). ¹⁹F NMR (377 MHz, METHANOL-d₄), δ=−120.734.

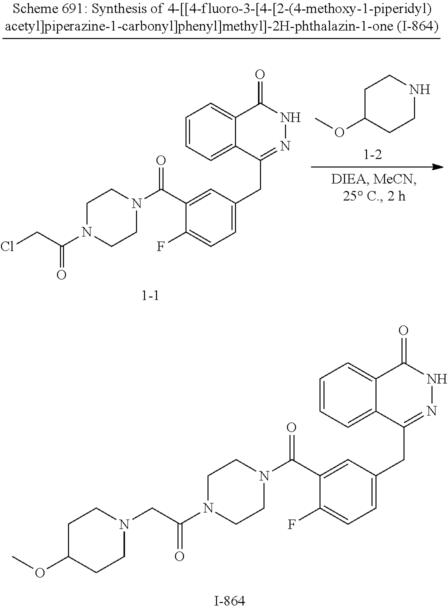

To a solution of Intermediate 1-1 (50 mg, 112.90 μmol, 1 eq) and Intermediate 1-2 (39.01 mg, 338.70 μmol, 3 eq) in MeCN (0.5 mL) was added DIEA (29.18 mg, 225.80 μmol, 39.33 μL, 2 eq). The mixture was stirred at 25° C. for 2 hr. The residue was concentrated under reduced pressure to give a residue. The crude product was purified by reversed phase chromatography (0.10% FA). The eluent was concentrated under reduced pressure to remove ACN, then lyophilized to afford product. I-864 (38 mg, 66.78 μmol, 59.15% yield, 99.754% purity, FA salt) was obtained as a yellow solid. LCMS (Method D): Rt=0.277 min, [M+H]⁺=522.3. ¹H NMR (400 MHz, METHANOL-d4) δ=8.47 (s, 1H), 8.36 (d, J=7.6 Hz, 1H), 7.98-7.92 (m, 1H), 7.91-7.78 (m, 2H), 7.54-7.45 (m, 1H), 7.37 (br t, J=5.6 Hz, 1H), 7.16 (t, J=9.2 Hz, 1H), 4.38 (s, 2H), 3.85-3.72 (m, 3H), 3.71-3.59 (m, 3H), 3.57-3.40 (m, 3H), 3.37 (s, 1H), 3.35 (d, J=3.2 Hz, 3H), 3.13-3.04 (m, 2H), 2.79 (br d, J=8.8 Hz, 2H), 2.05-1.71 (m, 5H). ¹⁹F NMR (377 MHz, METHANOL-d4) δ=−120.74.

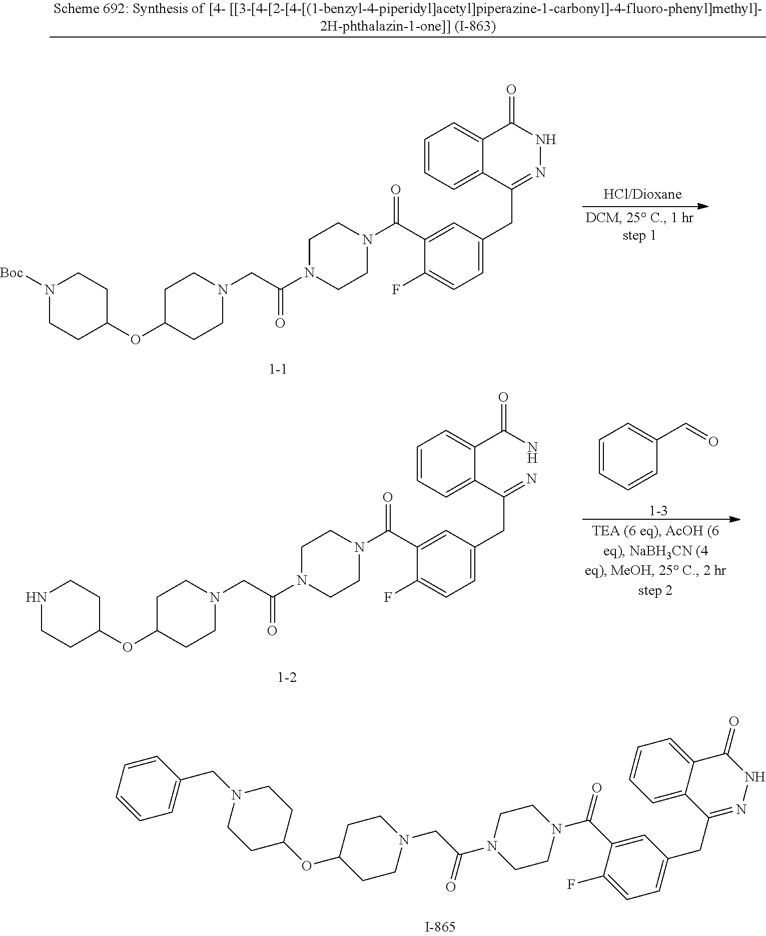

### Step 1: Synthesis of Intermediate 1-2

To a solution of Intermediate 1-1 (50 mg, 72.38 μmol, 1 eq) in DCM (0.5 mL) was added HCl/dioxane (2 M, 0.5 mL, 13.82 eq). The mixture was stirred at 25° C. for 1 hr. The reaction mixture was filtered and concentrated under reduced pressure to give a residue. The crude product was used in the next step without further purification. Intermediate 1-2 (69 mg, crude, HCl salt) was obtained as a white solid. LCMS (Method D): Rt=0.237 min, [M+H]⁺=591.4.

### Step 2: Synthesis of I-865

To a solution of Intermediate 1-2 (69 mg, 110.02 μmol, 1 eq, HCl salt) in MeOH (0.7 mL) was added TEA (66.80 mg, 660.13 μmol, 91.88 μL, 6 eq) and the mixture was stirred at 25° C. for 10 min. Then AcOH (39.64 mg, 660.13 μmol, 37.79 μL, 6 eq) and Intermediate 1-3 (14.01 mg, 132.03 μmol, 13.34 μL, 1.2 eq) were added to the mixture. The mixture was stirred at 25° C. for 30 min. Finally, NaBH₃CN (27.66 mg, 440.09 μmol, 4 eq) was added to the mixture and it was stirred at 25° C. for 1 hr. The reaction mixture was diluted with H₂O (1 mL). Then the mixture was extracted with DCM (5 mL*3). The combined organic layers were dried over Na₂SO₄, filtered and concentrated under reduced pressure to give a residue. The crude product was purified by reversed-phase HPLC (column: CD01-Phenomenex luna C18 150*25*10 um; mobile phase: [water(TFA)-ACN]; gradient: 12%-42% B over 10 min) followed by lyophilization to give product. I-865 (58.44 mg, 73.27 μmol, 45.95% yield, 99.657% purity, TFA salt) was obtained as a white solid. LCMS (Method D): Rt=0.265 min, [M+H]⁺=681.2. ¹HNMR (400 MHz, METHANOL-d4) δ=8.36 (d, J=7.8 Hz, 1H), 7.98-7.92 (m, 1H), 7.91-7.79 (m, 2H), 7.50 (s, 6H), 7.40-7.31 (m, 1H), 7.17 (t, J=9.0 Hz, 1H), 4.38 (s, 2H), 4.36-4.18 (m, 4H), 3.99-3.60 (m, 6H), 3.58-3.44 (m, 4H), 3.35-3.3.34 (m, 5H), 3.30-3.20 (m, 2H), 3.12-3.00 (m, 1H), 2.27-2.02 (m, 5H), 1.99-1.64 (m, 3H). ¹⁹F NMR (377 MHz, METHANOL-d4) δ=−76.90, −120.84.

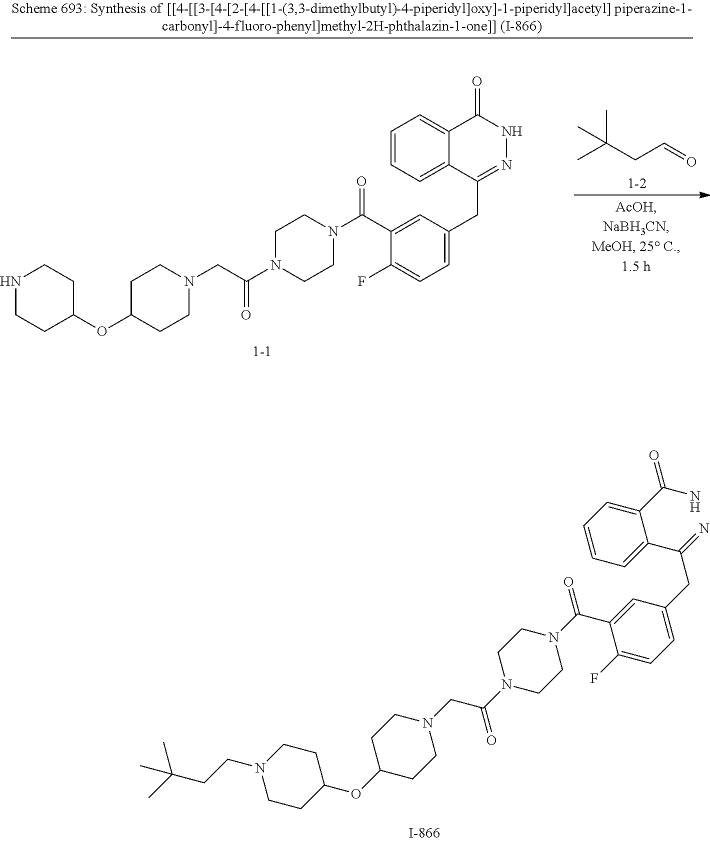

To a solution of Intermediate 1-1 (100 mg, 159.45 μmol, 1 eq, HCl salt) and Intermediate 1-2 (19.16 mg, 191.34 μmol, 24.02 μL, 1.2 eq) in MeOH (1 mL) was added AcOH (957.54 g, 15.95 μmol, 9.13e−1 μL, 0.1 eq) and the mixture was stirred at 25° C. for 1 hr, and then NaBH₃CN (30.06 mg, 478.36 μmol, 3 eq) was added to the mixture. The resulting mixture was stirred at 25° C. for 0.5 hr. The mixture was filtered and concentrated under reduced pressure to give a residue. The residue was purified by prep-HPLC (column: CD02-Waters Xbidge BEH C18 150*25*10 um; mobile phase: [water (NH₄HCO₃)-ACN]; gradient: 22%-52% B over 10 min) and concentrated under reduced pressure to remove ACN and lyophilized to give I-866 (29.78 mg, 44.13 μmol, 27.68% yield, 100% purity) as a white solid. LCMS (Method D): Retention time: 0.283 min, [M+H]⁺=675.2. ¹H NMR (400 MHz, METHANOL-d4) δ=8.40-8.32 (m, 1H), 7.99-7.92 (m, 1H), 7.91-7.79 (m, 2H), 7.52-7.45 (m, 1H), 7.41-7.34 (m, 1H), 7.16 (t, J=8.8 Hz, 1H), 4.39 (s, 2H), 3.83-3.64 (m, 4H), 3.59-3.45 (m, 4H), 3.33 (s, 1H), 3.30-3.12 (m, 3H), 2.78 (d, J=12.4 Hz, 4H), 2.41-2.36 (m 2H), 2.24 (d, J=10.4 Hz, 4H), 1.88 (d, J=13.2 Hz, 4H), 1.66-1.49 (m, 4H), 1.47-1.37 (m, 2H), 0.92 (s, 9H). ¹⁹F NMR (400 MHz, METHANOL-d4) δ=−120.787.

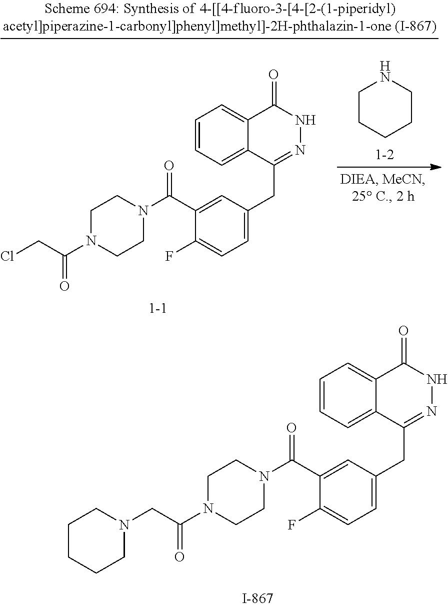 [SMILES: O=C(CCl)N1CCN(C(=O)c2cc(Cc3n[nH]c(=O)c4ccccc34)ccc2F)CC1 ; C1CCNCC1 ; O=C(CN1CCCCC1)N1CCN(C(=O)c2cc(Cc3n[nH]c(=O)c4ccccc34)ccc2F)CC1]

To a solution of Intermediate 1-1 (50 mg, 112.90 μmol, 1 eq) and Intermediate 1-2 (28.84 mg, 338.70 μmol, 33.45 μL, 3 eq) in MeCN (0.5 mL) was added DIEA (29.18 mg, 225.80 μmol, 39.33 μL, 2 eq). The mixture was stirred at 25° C. for 2 hr. The residue was concentrated under reduced pressure to give a residue. The crude product was purified by reversed phase chromatography (0.1% FA). The eluent was concentrated under reduced pressure to remove ACN, then lyophilized to afford product. I-867 (30 mg, 55.81 μmol, 49.43% yield, 100% purity, FA salt) was obtained as a white solid. LCMS (Method D): Rt=0.274 min, [M+H]⁺=492.2. ¹H NMR (400 MHz, METHANOL-d4) δ=8.49 (s, 1H), 8.37 (d, J=7.6 Hz, 1H), 7.98-7.92 (m, 1H), 7.91-7.79 (m, 2H), 7.56-7.44 (m, 1H), 7.41-7.32 (m, 1H), 7.17 (t, J=9.2 Hz, 1H), 4.39 (s, 2H), 3.92 (s, 1H), 3.86-3.79 (m, 2H), 3.78-3.67 (m, 2H), 3.59-3.52 (m, 2H), 3.45-3.34 (m, 2H), 3.01 (br s, 4H), 1.89-1.53 (m, 7H). ¹⁹F NMR (377 MHz, METHANOL-d4) δ=−120.75.

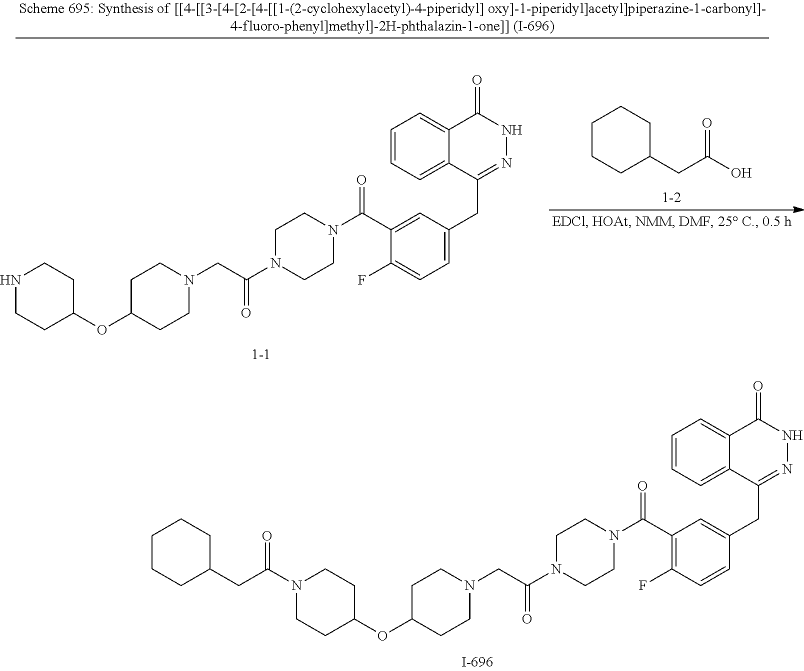 [SMILES: O=C(CN1CCC(OC2CCNCC2)CC1)N1CCN(C(=O)c2cc(Cc3n[nH]c(=O)c4ccccc34)ccc2F)CC1 ; O=C(O)CC1CCCCC1 ; O=C(CC1CCCCC1)N1CCC(OC2CCN(CC(=O)N3CCN(C(=O)c4cc(Cc5n[nH]c(=O)c6ccccc56)ccc4F)CC3)CC2)CC1]

To a solution of intermediate 1-1 (90 mg, 143.51 μmol, 1 eq, HCl salt) and intermediate 1-2 (26.53 mg, 186.56 μmol, 26.34 μL, 1.3 eq) in DMF (0.9 mL) was added EDCI (82.53 mg, 430.52 μmol, 3 eq), HOAt (19.53 mg, 143.51 μmol, 20.07 μL, 1 eq) and NMM (72.58 mg, 717.53 μmol, 78.89 μL, 5 eq). The mixture was stirred at 25° C. for 0.5 hr. The reaction mixture was poured into H₂O (5 mL) and extracted with EA (2 mL*3). The combined organic layers were dried over Na₂SO₄, filtered and concentrated under reduced pressure to give a residue. The residue was purified by reversed-phase chromatography (0.1% FA). The eluent was concentrated to remove MeCN and then lyophilized to afford I-696 (35.05 mg, 45.95 mol, 32.02% yield, 99.76% purity, FA salt) as a white solid. LCMS (Method D): Rt=0.371 min, [M+H]⁺=715.4. ¹H NMR (400 MHz, METHANOL-d4) δ=8.47 (s, 1H), 8.43-8.28 (m, 1H), 7.94 (d, J=3.6 Hz, 1H), 7.91-7.78 (m, 2H), 7.50-7.48 (m, 1H), 7.41-7.32 (m, 1H), 7.17 (t, J=8.8 Hz, 1H), 4.39 (s, 2H), 3.97-3.88 (m, 1H), 3.83-3.72 (m, 4H), 3.71-3.60 (m, 3H), 3.58-3.44 (m, 4H), 3.38-3.33 (m, 2H), 3.02-2.87 (m, 2H), 2.68-2.43 (m, 2H), 2.27 (d, J=6.0 Hz, 2H), 2.02-1.77 (m, 5H), 1.77-1.62 (m, 8H), 1.56-1.43 (m, 2H), 1.38-1.13 (m, 4H), 1.06-0.94 (m, 2H). ¹⁹F NMR (377 MHz, METHANOL-d4) δ=−120.794.

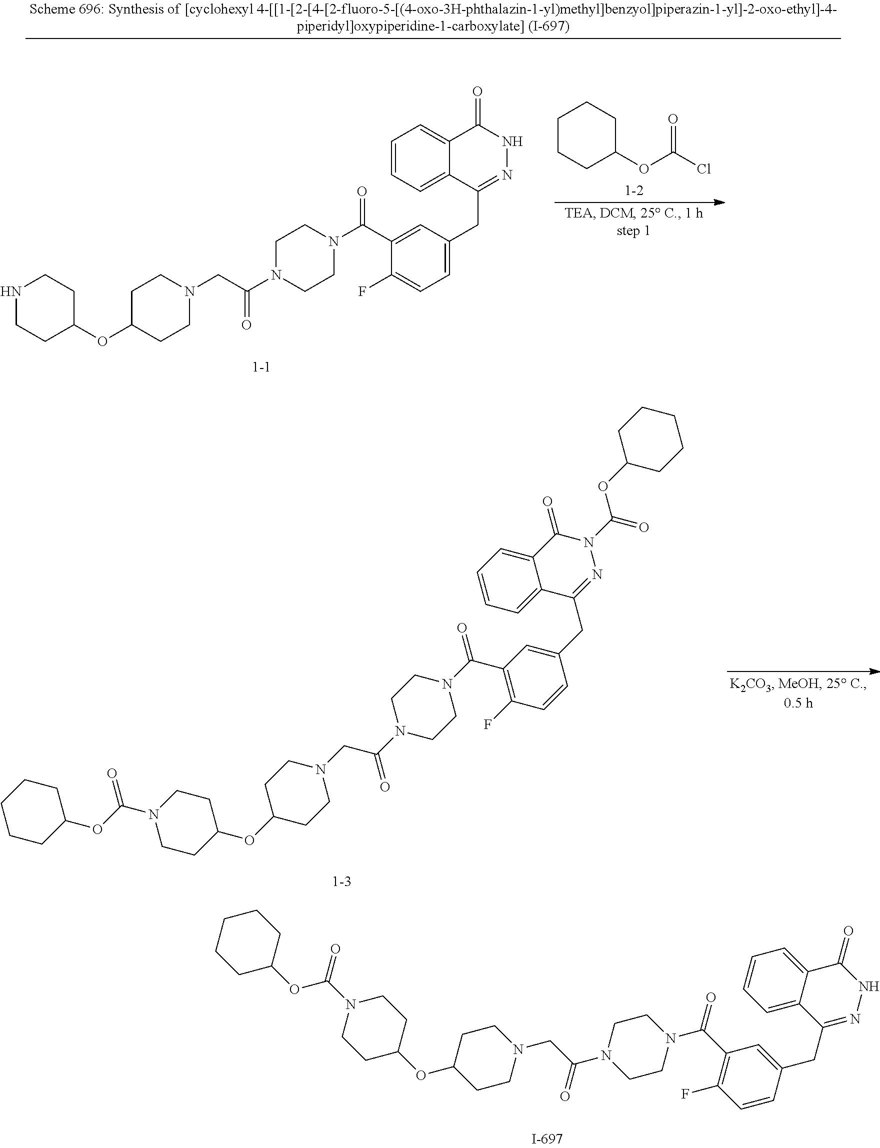 [SMILES: O=C(CN1CCC(OC2CCNCC2)CC1)N1CCN(C(=O)c2cc(Cc3n[nH]c(=O)c4ccccc34)ccc2F)CC1 ; O=C(Cl)OC1CCCCC1 ; O=C(CN1CCC(OC2CCN(C(=O)OC3CCCCC3)CC2)CC1)N1CCN(C(=O)c2cc(Cc3nn(C(=O)OC4CCCCC4)c(=O)c4ccccc34)ccc2F)CC1 ; O=C(CN1CCC(OC2CCN(C(=O)OC3CCCCC3)CC2)CC1)N1CCN(C(=O)c2cc(Cc3n[nH]c(=O)c4ccccc34)ccc2F)CC1]

### Step 1: Synthesis of Intermediate 1-3

To a solution of Intermediate 1-1 (80 mg, 127.56 μmol, 1 eq, HCl salt) and Intermediate 1-2 (20.74 mg, 127.56 μmol, 1 eq) in DCM (1 mL) was added TEA (25.82 mg, 255.12 μmol, 35.51 μL, 2 eq). The mixture was stirred at 25° C. for 0.5 hr. The reaction mixture was concentrated under reduced pressure to give a residue. The residue was used for the next step without further purification. Intermediate 1-3 (85 mg, 100.83 μmol, 79.05% yield) was obtained as a white solid. LCMS (Method D): Retention time: 0.436 min, [M+H]⁺=843.7.

### Step 2: Synthesis of I-697

To a solution of Intermediate 1-3 (80 mg, 94.90 μmol, 1 eq) in MeOH (1 mL) was added K₂CO₃ (26.23 mg, 189.80 μmol, 2 eq). The mixture was stirred at 25° C. for 0.5 hr. The reaction mixture was filtered and concentrated under reduced pressure to give a residue. The residue was purified by reversed phase chromatography (0.1% FA condition). The eluent was lyophilized to give product. I-697 (30.32 mg, 39.37 μmol, 41.48% yield, 99.049% purity, FA salt) was obtained as a white solid. LCMS (Method D): Retention time: 0.368 min, [M+H]⁺=717.5. ¹H NMR (400 MHz, METHANOL-d4) δ=8.48 (s, 1H), 8.37 (d, J=7.6 Hz, 1H), 7.98-7.92 (m, 1H), 7.91-7.80 (m, 2H), 7.49 (s, 1H), 7.37 (t, J=5.2 Hz, 1H), 7.16 (t, J=8.8 Hz, 1H), 4.63-4.59 (m, 1H), 4.39 (s, 2H), 3.77 (d, J=14.8 Hz, 4H), 3.72-3.61 (m, 5H), 3.56-3.46 (m, 3H), 3.34 (s, 1H), 3.25-3.16 (m, 2H), 3.02-2.98 (m, 2H), 2.75-2.54 (m, 2H), 2.01-1.90 (m, 2H), 1.83 (d, J=5.6 Hz, 4H), 1.73 (d, J=6.0 Hz, 4H), 1.58-1.28 (m, 9H). ¹⁹F NMR (400 MHz, METHANOL-d₄) δ=−120.772.

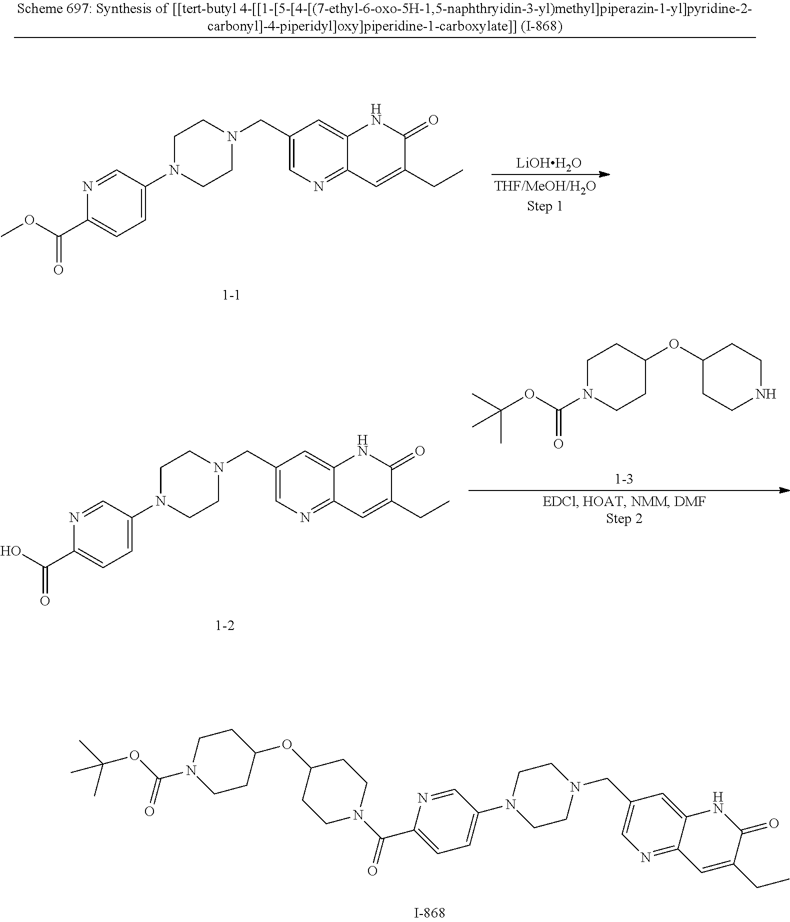

### Step 1: Synthesis of Intermediate 1-1.

To a solution of Intermediate 1-1 (1 g, 2.45 mmol, 1 eq) in MeOH (0.5 mL), THF (0.5 mL), and H₂O (0.5 mL) was added LiOH·H₂O (308.96 mg, 7.36 mmol, 3 eq). The mixture was stirred at 25° C. for 0.5 hr. The reaction mixture was concentrated in vacuo to remove THF and MeOH to give a residue, and the residue was diluted with water (5 mL) and extracted with DCM (5 mL*3), then the aqueous phase was acidified with hydrochloric acid (1 M) to pH=5-6, the mixture was extracted with DCM (5 mL*3). The combined organic layer was dried over Na₂SO₄, filtered and concentrated in vacuo to give a residue. The residue was used in the next step directly without further purification. Intermediate 1-2 (1 g, crude) was obtained as a white solid. LCMS (Method D): Rt=0.168 min, [M+H]⁺=394.0.

### Step 2: Synthesis of I-868.

To a solution of Intermediate 1-2 (40 mg, 101.67 μmol, 1 eq) and Intermediate 1-3 (43.37 mg, 152.50 μmol, 1.5 eq) in DMF (0.5 mL) was added EDCI (58.47 mg, 305.00 μmol, 3 eq), HOAt (13.84 mg, 101.67 μmol, 14.22 μL, 1 eq), and NMM (51.42 mg, 508.34 μmol, 55.89 μL, 5 eq). The mixture was stirred at 25° C. for 1 hr. The mixture was diluted with water (5 mL) and extracted with DCM (5 mL*3). The combined organic layer was dried over Na₂SO₄, filtered and concentrated in vacuo to give a residue. The residue was purified by prep-HPLC (column: CD01-Phenomenex luna C18 150*25*10 um; mobile phase: [water (FA)-ACN]; gradient: 8%-38% B over 11 min). Then the eluent was concentrated in vacuo and lyophilized. I-868 (10.45 mg, 15.74 μmol, 15.48% yield, 99.375% purity) was obtained as a white solid. LCMS (Method D): Rt=0.341 min, [M+H]⁺=660.2. ¹H NMR (400 MHz, METHANOL-d4) δ=8.49 (d, J=1.6 Hz, 1H), 8.24 (s, 1H), 7.84 (s, 1H), 7.77 (d, J=1.2 Hz, 1H), 7.51-7.45 (m, 1H), 7.43-7.40 (m, 1H), 4.02-4.00 (m, 1H), 3.79-3.77 (m, 2H), 3.74 (s, 3H), 3.72-3.67 (m, 2H), 3.55-3.47 (m, 1H), 3.39 (s, 1H), 3.38 (s, 2H), 3.37 (s, 1H), 3.19-3.11 (m, 2H), 2.71-2.67 (m, 5H), 2.66-2.64 (m, 1H), 1.97-1.89 (m, 1H), 1.86-1.78 (m, 3H), 1.69-1.55 (m, 2H), 1.45 (s, 12H), 1.29 (t, J=7.6 Hz, 3H).

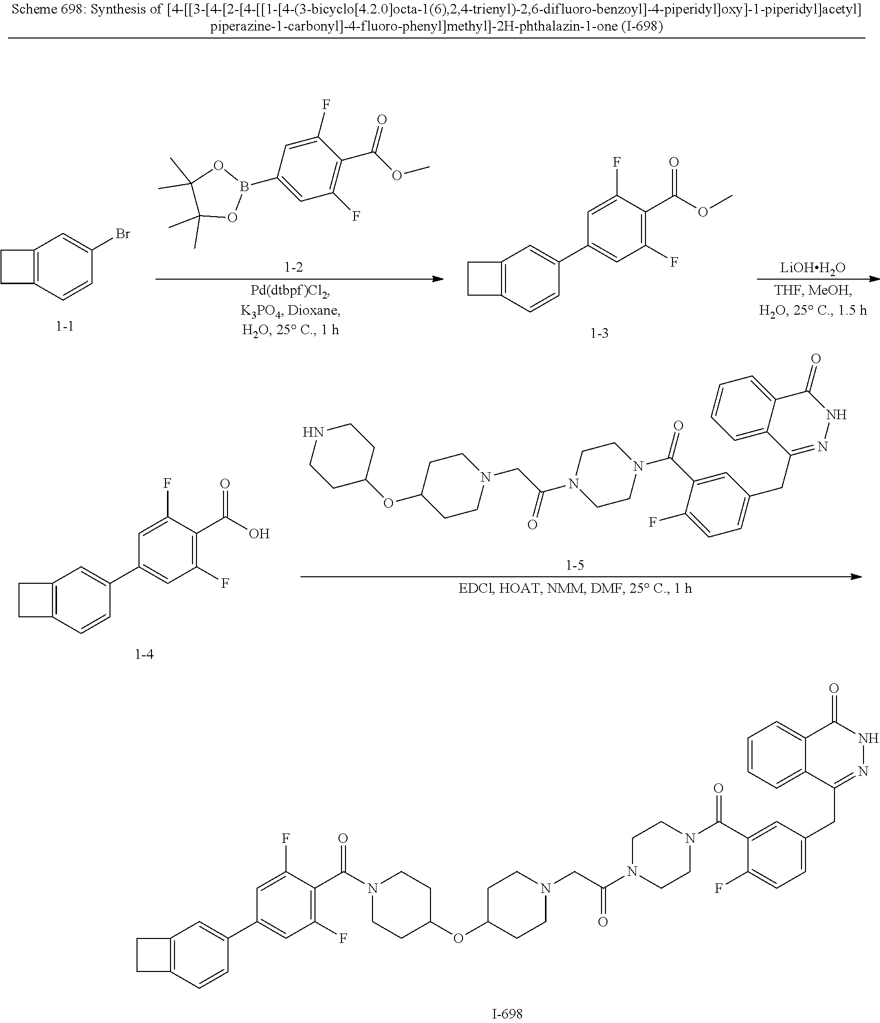

### Step 1: Synthesis of Intermediate 1-3

A mixture of Intermediate 1-1 (0.3 g, 1.64 mmol, 1 eq), Intermediate 1-2 (488.55 mg, 1.64 mmol, 1 eq), Pd(dtbpf)Cl₂ (106.82 mg, 163.89 μmol, 0.1 eq), and K₃PO₄ (1.04 g, 4.92 mmol, 3 eq) in dioxane (3 mL) and H₂O (0.6 mL) was degassed and purged with N₂ 3 times, and then the mixture was stirred at 80° C. for 1 hr under N₂ atmosphere. The reaction mixture was diluted with water (10 mL) and extracted with DCM (10 mL*3). The combined organic layers were dried over Na₂SO₄, filtered and concentrated under reduced pressure to give a residue. The residue was purified by flash silica gel chromatography (ISCO; 40 g SepaFlash Silica Flash Column, Eluent of 0˜100% Petroleum ether/Ethyl acetate @60 mL/min). The eluent was concentrated under reduced pressure to give a residue. Intermediate 1-3 (610 mg, crude) was obtained as a yellow oil. LCMS (Method D): Retention time: 0.450 min, [M+H]⁺=275.1.

### Step 2: Synthesis of Intermediate 1-4

To a solution of Intermediate 1-3 (600 mg, 2.19 mmol, 1 eq) in THF (2 mL), MeOH (2 mL), and H₂O (2 mL) was added LiOH H₂O (183.61 mg, 4.38 mmol, 2 eq). The mixture was stirred at 25° C. for 1.5 hr. The mixture was adjusted to pH=5 with citric acid and extracted with EA (10 mL*3). The combined organic layers were dried over Na₂SO₄, filtered and concentrated under reduced pressure to give a residue. The residue was used for the next step without further purification. Intermediate 1-4 (0.5 g, 1.92 mmol, 87.82% yield) was obtained as a yellow oil. LCMS (Method D): Retention time: 0.434 min, [M+H]⁺=260.7. ¹H NMR (400 MHz, METHANOL-d4) δ=7.45-7.38 (m, 1H), 7.27 (s, 1H), 7.14-7.07 (m, 3H), 3.21 (s, 4H). ¹⁹F NMR (377 MHz, METHANOL-d₄) δ=−116.006.

### Step 3: Synthesis of I-698

To a solution of Intermediate 1-4 (50 mg, 192.13 μmol, 1 eq) in DMF (1 mL) was added HOAt (26.15 mg, 192.13 μmol, 26.88 μL, 1 eq), EDCI (110.50 mg, 576.40 μmol, 3 eq), NMM (97.17 mg, 960.67 mol, 105.62 μL, 5 eq), and Intermediate 1-5 (120.50 mg, 192.13 μmol, 1 eq, HCl salt). The mixture was stirred at 25° C. for 1 hr. The reaction mixture was diluted with water (5 mL) and extracted with DCM (5 mL*3). The combined organic layers were dried over Na₂SO₄, filtered and concentrated under reduced pressure to give a residue. The residue was purified by prep-HPLC (column: CD01-Phenomenex luna C18 150*25*10 um; mobile phase: [water(FA)-ACN]; gradient: 22%-52% B over 10 min). The eluent was lyophilized to give product. I-698 (39.86 mg, 45.35 μmol, 23.60% yield, FA salt) was obtained as a white solid. LCMS (Method D): Retention time: 0.400 min, [M+H]⁺=833.5. ¹H NMR (400 MHz, METHANOL-d4) δ=8.49 (s, 1H), 8.37 (d, J=7.6 Hz, 1H), 7.98-7.92 (m, 1H), 7.91-7.80 (m, 2H), 7.47 (d, J=8.0 Hz, 2H), 7.40-7.35 (m, 1H), 7.35-7.28 (m, 3H), 7.21-7.12 (m, 2H), 4.38 (s, 2H), 4.10-4.00 (m, 1H), 3.84-3.72 (m, 3H), 3.71-3.56 (m, 5H), 3.55-3.46 (m, 3H), 3.39 (s, 1H), 3.37-3.32 (m, 3H), 3.23 (s, 4H), 2.91 (s, 2H), 2.50 (d, J=11.2 Hz, 2H), 1.99-1.81 (m, 4H), 1.76-1.52 (m, 4H). ¹⁹F NMR (377 MHz, METHANOL-d₄) δ=−115.525, −115.600, −120.734, −120.652.

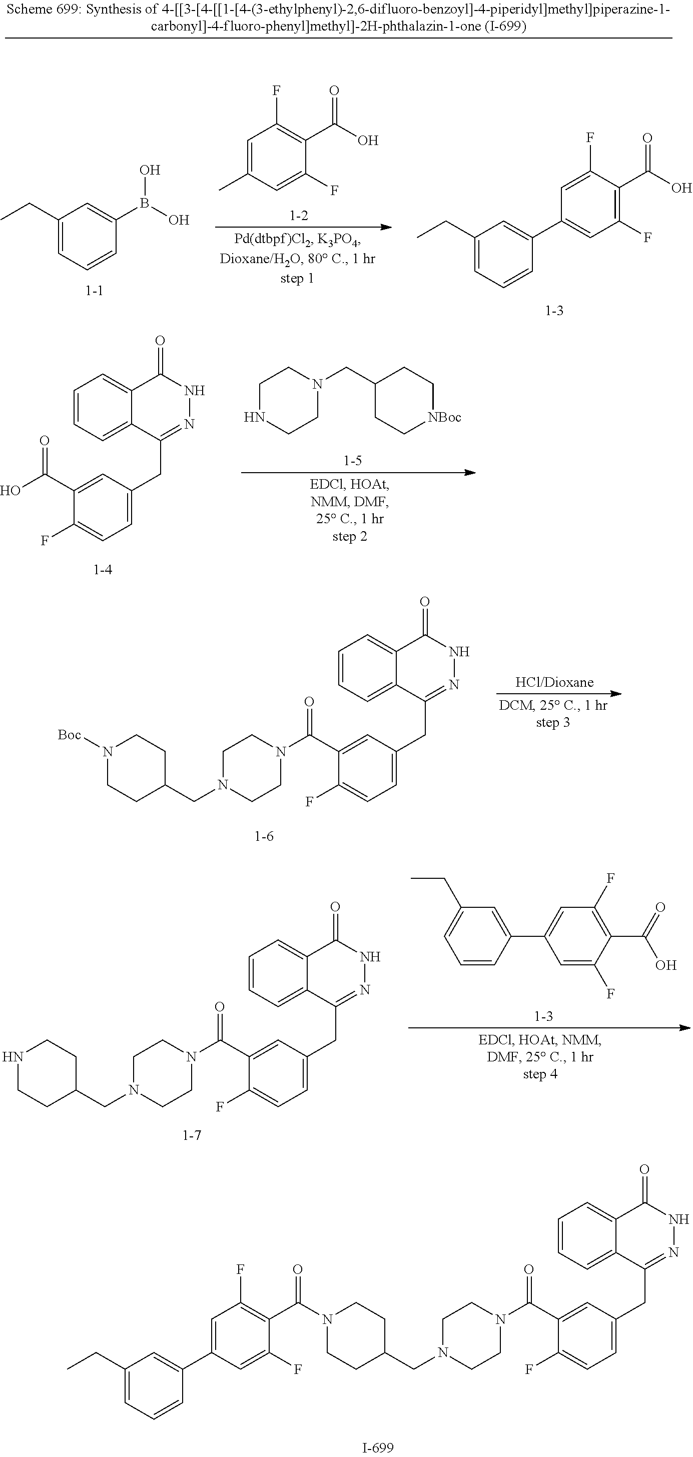

### Step 1: Synthesis of Intermediate 1-3

A mixture of Intermediate 1-1 (759.41 mg, 5.06 mmol, 1.2 eq), Intermediate 1-2 (1 g, 4.22 mmol, 1 eq), Pd(dtbpf)Cl₂ (275.00 mg, 421.94 μmol, 0.1 eq), and K₃PO₄ (2.69 g, 12.66 mmol, 3 eq) in dioxane (10 mL) and H₂O (2 mL) was degassed and purged with N₂ 3 times, and then the mixture was stirred at 80° C. for 1 hr under an N₂ atmosphere. The mixture was adjusted to pH=10 with a saturated NaOH solution and washed with EA (20 mL*3), then the aqueous phase was adjusted to pH=5 with HCl (2M) and extracted with EA (20 mL*4), dried over Na₂SO₄, filtered and concentrated under reduced pressure to give a residue. The residue was purified by flash silica gel chromatography (ISCO; 60 g SepaFlash Silica Flash Column, Eluent of 0˜100% Petroleum ether/Ethyl acetate@60 mL/min). The eluent was concentrated under reduced pressure to give Intermediate 1-3 as a brown solid. LCMS (Method D): Rt=0.455 min, [M+H]⁺=263.1. ¹H NMR (400 MHz, METHANOL-d4) δ=7.53-7.42 (m, 2H), 7.38 (t, J=7.6 Hz, 1H), 7.35-7.21 (m, 3H), 2.75-2.69 (m, 2H), 1.27 (t, J=7.6 Hz, 3H). ¹⁹F NMR (377 MHz, METHANOL-d₄), δ=−112.424.

### Step 2: Synthesis of Intermediate 1-6

To a solution of Intermediate 1-4 (200 mg, 670.54 μmol, 1 eq) in DMF (2 mL) was added HOAt (91.27 mg, 670.54 μmol, 93.80 μL, 1 eq), EDCI (385.63 mg, 2.01 mmol, 3 eq), NMM (339.11 mg, 3.35 mmol, 368.60 μL, 5 eq) and Intermediate 1-5 (228.04 mg, 804.65 μmol, 1.2 eq). The mixture was stirred at 25° C. for 1 hr. The residue was diluted with H₂O (5 mL) and extracted with EA (5 mL*3), dried over Na₂SO₄, filtered and concentrated under reduced pressure to give a residue. The residue was purified by flash silica gel chromatography (ISCO; 20 g SepaFlash Silica Flash Column, Eluent of 0˜100% Dichloromethane/Methanol @40 mL/min). The eluent was concentrated under reduced pressure to give Intermediate 1-6 (300 mg, 530.95 μmol, 79.18% yield, 99.758% purity) as a white solid. LCMS (Method D): Rt=0.324 min, [M+H]⁺=564.3.

### Step 3: Synthesis of Intermediate 1-7

To a solution of Intermediate 1-6 (200 mg, 354.82 μmol, 1 eq) in DCM (2 mL) was added HCl/dioxane (2 M, 1 mL, 5.64 eq). The mixture was stirred at 25° C. for 1 hr. The reaction mixture was concentrated by reduced pressure to obtain the crude product. The crude product was used in the next step without further purification. Intermediate 1-7 (150 mg, 296.19 μmol, 83.48% yield, 98.733% purity, HCl salt) was obtained as a white solid. LCMS (Method D): Rt=0.213 min, [M+H]⁺=464.1.

### Step 4: Synthesis of I-699

To a solution of Intermediate 1-3 (30 mg, 114.39 μmol, 1 eq) in DMF (5 mL) was added HOAt (15.57 mg, 114.39 μmol, 16.00 μL, 1 eq), EDCI (65.79 mg, 343.18 μmol, 3 eq), NMM (57.85 mg, 571.97 μmol, 62.88 μL, 5 eq) and Intermediate 1-7 (68.64 mg, 137.27 μmol, 1.2 eq, HCl salt). The mixture was stirred at 25° C. for 1 hr. The residue was diluted with H₂O (5 mL) and extracted with EA (5 mL*3), dried over Na₂SO₄, filtered and concentrated under reduced pressure to give a residue. The crude product was purified by reversed-phase HPLC (column: CD01-Phenomenex luna C18 150*25*10 um; mobile phase: [water (FA)-ACN]; gradient: 25%-55% B over 8 min). The eluent was concentrated in vacuum to remove ACN. Then it was lyophilized. I-699 (57.89 mg, 76.54 μmol, 66.91% yield, 99.672% purity, FA salt) was obtained as a white solid. LCMS (Method D): Rt=0.376 min, [M+H]⁺=708.4. ¹H NMR (400 MHz, METHANOL-d4) δ=8.39-8.35 (m, 1H), 8.27-8.19 (m, 1H), 7.96-7.91 (m, 1H), 7.89-7.79 (m, 2H), 7.54-7.43 (m, 3H), 7.42-7.33 (m, 3H), 7.32-7.25 (m, 2H), 7.14 (t, J=8.8 Hz, 1H), 4.69 (d, J=12.8 Hz, 1H), 4.37 (s, 2H), 3.84-3.68 (m, 2H), 3.62 (d, J=13.6 Hz, 1H), 3.26 (s, 2H), 3.24-3.12 (m, 1H), 2.97-2.87 (m, 1H), 2.76-2.70 (m, 2H), 2.52 (br s, 2H), 2.30 (d, J=6.8 Hz, 2H), 2.29-2.22 (m, 2H), 2.00-1.87 (m, 2H), 1.83 (d, J=13.6 Hz, 1H), 1.28 (t, J=7.6 Hz, 3H), 1.24-1.09 (m, 2H). ¹⁹F NMR (377 MHz, METHANOL-d₄), δ=−115.334, −115.426, −120.815.

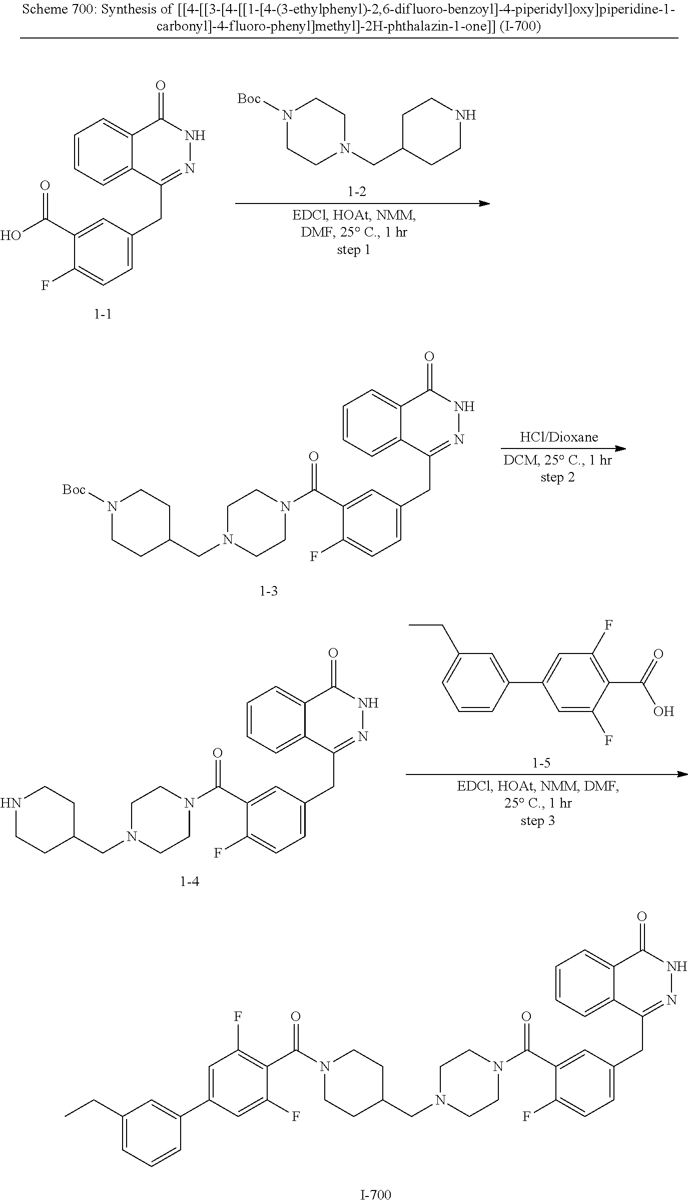 [SMILES: O=C(O)c1cc(Cc2n[nH]c(=O)c3ccccc23)ccc1F ; CC(C)(C)OC(=O)N1CCN(CC2CCNCC2)CC1 ; CC(C)(C)OC(=O)N1CCC(CN2CCN(C(=O)c3cc(Cc4n[nH]c(=O)c5ccccc45)ccc3F)CC2)CC1 ; O=C(c1cc(Cc2n[nH]c(=O)c3ccccc23)ccc1F)N1CCN(CC2CCNCC2)CC1 ; CCc1cccc(-c2cc(F)c(C(=O)O)c(F)c2)c1 ; CCc1cccc(-c2cc(F)c(C(=O)N3CCC(CN4CCN(C(=O)c5cc(Cc6n[nH]c(=O)c7ccccc67)ccc5F)CC4)CC3)c(F)c2)c1]

### Step 1: Synthesis of Intermediate 1-3.

To a solution of Intermediate 1-1 (200 mg, 670.54 μmol, 1 eq) in DMF (3 mL) was added EDCI (385.63 mg, 2.01 mmol, 3 eq), HOAt (91.27 mg, 670.54 μmol, 93.80 μL, 1 eq), NMM (339.11 mg, 3.35 mmol, 368.60 μL, 5 eq) and Intermediate 1-2 (228.84 mg, 804.65 μmol, 1.2 eq). The mixture was stirred at 25° C. for 1 hr. The reaction mixture was poured into water (10 mL) and extracted with EA (5 mL*3). The combined organic layers were dried over Na₂SO₄, filtered and concentrated under reduced pressure to give a residue. The residue was purified by flash silica gel chromatography (ISCO; 12 g SepaFlash Silica Flash Column, Eluent of 0˜100% DCM/MeOH@18 mL/min, DCM/MeOH=10:1, Rf=0.6) and the eluent was concentrated under reduced pressure to give Intermediate 1-3 (350 mg, 619.86 μmol, 92.44% yield) as a white solid.

LCMS (Method D): Retention time: 0.454 min, [M-Boc+H]⁺=465.0.

### Step 2: Synthesis of Intermediate 1-4.

To a solution of Intermediate 1-3 (300 mg, 531.31 μmol, 1 eq) in DCM (1 mL) was added HCl/dioxane (2 M, 3.00 mL, 11.29 eq). The mixture was stirred at 25° C. for 0.5 hr. The mixture was concentrated under reduced pressure to give Intermediate 1-4 (300 mg, crude, HCl salt) as a white solid. LCMS (Method D): Retention time: 0.277 min, [M+H]⁺=465.2.

### Step 3: Synthesis of I-700.

To a solution of Intermediate 1-5 (52.35 mg, 199.60 μmol, 1 eq) in DMF (1 mL) was added EDCI (114.79 mg, 598.81 μmol, 3 eq), HOAt (27.17 mg, 199.60 μmol, 27.92 μL, 1 eq), NMM (100.95 mg, 998.02 μmol, 109.72 μL, 5 eq) and Intermediate 1-4 (100 mg, 199.60 μmol, 1 eq, HCl salt). The mixture was stirred at 25° C. for 1 hr. The reaction was poured into water (5 mL) and extracted with EA (3 mL*3). The combined organic layers were dried over Na₂SO₄, filtered and concentrated under reduced pressure to give a residue. The residue was purified by prep-HPLC (column: CD01-Phenomenex luna C18 150*25*10 um; mobile phase: [water (FA)-ACN]; gradient: 50%-80% B over 10 min) and concentrated under reduced pressure to remove CAN and lyophilized to give I-700 (25.02 mg, 34.93 μmol, 17.50% yield, 98.945% purity) as a white solid. LCMS (Method D): Retention time: 0.517 min, [M+H]⁺=709.3. ¹H NMR (400 MHz, METHANOL-d4) δ=8.42-8.34 (m, 1H), 7.99-7.93 (m, 1H), 7.92-7.80 (m, 2H), 7.55-7.45 (m, 3H), 7.44-7.37 (m, 3H), 7.35-7.29 (m, 2H), 7.19-7.13 (m, 1H), 4.39 (d, J=1.6 Hz, 2H), 4.12-3.96 (m, 2H), 3.89-3.77 (m, 2H), 3.73-3.53 (m, 3H), 3.51-3.40 (m, 1H), 3.29 (s, 1H), 3.23-3.12 (m, 1H), 2.78-2.72 (m, 2H), 2.04 (d, J=8.8 Hz, 1H), 2.00-1.83 (m, 3H), 1.79-1.58 (m, 4H), 1.56-1.39 (m, 1H), 1.30 (t, J=7.6 Hz, 3H). ¹⁹F NMR (400 MHz, METHANOL-d4) δ=−115.345, −121.132.

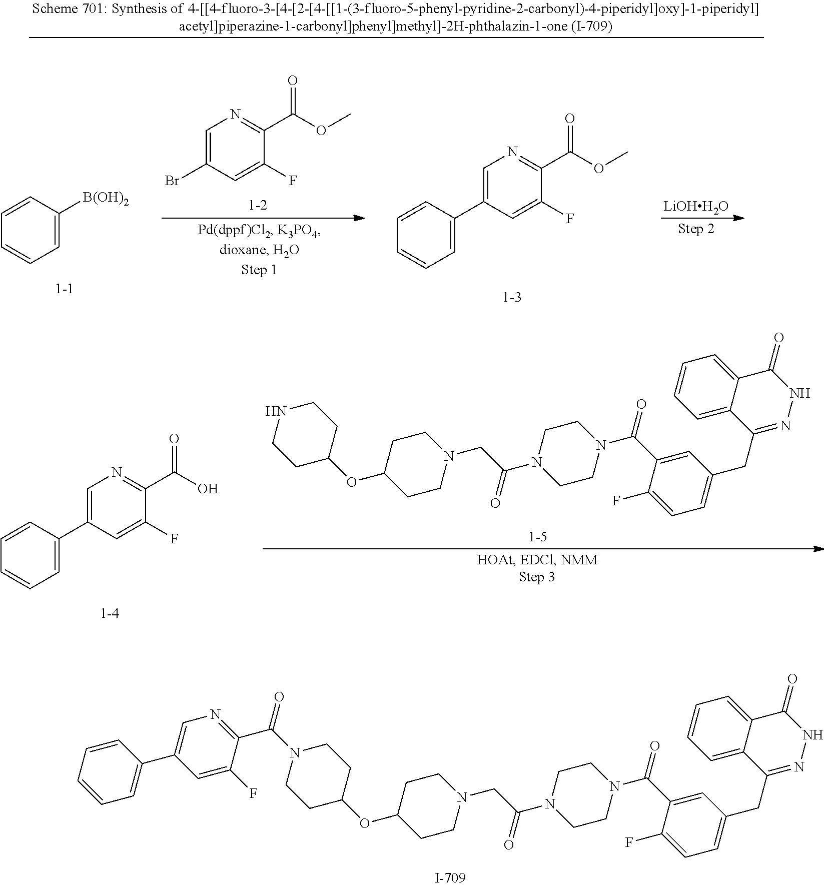

### Step 1: Synthesis of Intermediate 1-3.

To a mixture of Intermediate 1-1 (1 g, 8.20 mmol, 1 eq) and Intermediate 1-2 (1.92 g, 8.20 mmol, 1 eq) in dioxane (10 mL) and H₂O (2 mL) was added Pd(dppf)Cl₂ (600.11 mg, 820.15 μmol, 0.1 eq) and K₃PO₄ (5.22 g, 24.60 mmol, 3 eq), then the mixture was stirred at 80° C. for 1 hour under N₂. The reaction mixture was diluted with water (100 mL) and extracted with EA (30 mL*2). The combined organic layers were concentrated under reduced pressure to give a residue. The residue was purified by flash silica gel chromatography (ISCO; 40 g SepaFlash Silica Flash Column, Eluent of 0˜50% Ethyl acetate/Petroleum ether gradient @60 mL/min). The eluent was concentrated under reduced pressure to give Intermediate 1-3 (1.8 g, 7.78 mmol, 94.92% yield) as a white solid. LCMS (Method D): Retention time: 0.374 min, [M-Me+H]⁺=217.8.

### Step 2: Synthesis of Intermediate 1-4.

To a mixture of Intermediate 1-3 (1 g, 4.32 mmol, 1 eq) in THF (8 mL), MeOH (4 mL) and H₂O (2 mL) was added LiOH·H₂O (544.46 mg, 12.97 mmol, 3 eq), then the mixture was stirred at 20° C. for 1 hour. The reaction mixture was concentrated under reduced pressure to remove THF and MeOH then about 40 mL of 1 N HCl was added to adjust the pH to 1-2, the mixture was filtered, the solid was collected and dried under reduced pressure to give Intermediate 1-4 (480 mg, 2.21 mmol, 51.10% yield) as a white solid. LCMS (Method D): Retention time: 0.317 min, [M+H]⁺=218.1.

### Step 3: Synthesis of I-709.

To a mixture of Intermediate 1-4 (200 mg, 920.83 μmol, 1 eq) and Intermediate 1-5 (577.50 mg, 920.83 μmol, 1 eq, HCl salt) in DMF (5 mL) was added HOAt (125.34 mg, 920.83 μmol, 128.81 μL, 1 eq), EDCI (529.57 mg, 2.76 mmol, 3 eq) and NMM (465.70 mg, 4.60 mmol, 506.19 μL, 5 eq), then the mixture was stirred at 20° C. for 1 hour. The reaction mixture was quenched with water (50 mL) and extracted with EA (10 mL*3). The combined organic layers were concentrated under reduced pressure to give a residue. The crude product was purified by reversed-phase chromatography (0.1% FA condition). The eluent was concentrated under reduced pressure to remove MeCN and then lyophilized to give I-709 (267.07 mg, 310.33 μmol, 33.70% yield, 97.13% purity, FA salt) as a white solid. LCMS (Method D): Retention time: 0.356 min, [M+H]⁺=790.3. ¹H NMR (400 MHz, METHANOL-d4) δ=8.74 (s, 1H), 8.46 (s, 1H), 8.39-8.34 (m, 1H), 8.06-7.99 (m, 1H), 7.98-7.92 (m, 1H), 7.91-7.80 (m, 2H), 7.74 (d, J=7.2 Hz, 2H), 7.57-7.44 (m, 4H), 7.40-7.35 (m, 1H), 7.19-7.15 (m, 1H), 4.38 (s, 2H), 4.11-4.01 (m, 1H), 3.84-3.72 (m, 3H), 3.69-3.48 (m, 9H), 3.41-3.34 (m, 1H), 3.30-3.20 (m, 2H), 3.04-2.90 (m, 2H), 2.72-2.50 (m, 2H), 2.00-1.83 (m, 4H), 1.79-1.58 (m, 4H). ¹⁹F NMR (376 MHz, METHANOL-d4), δ=−120.777, 6=−127.704.

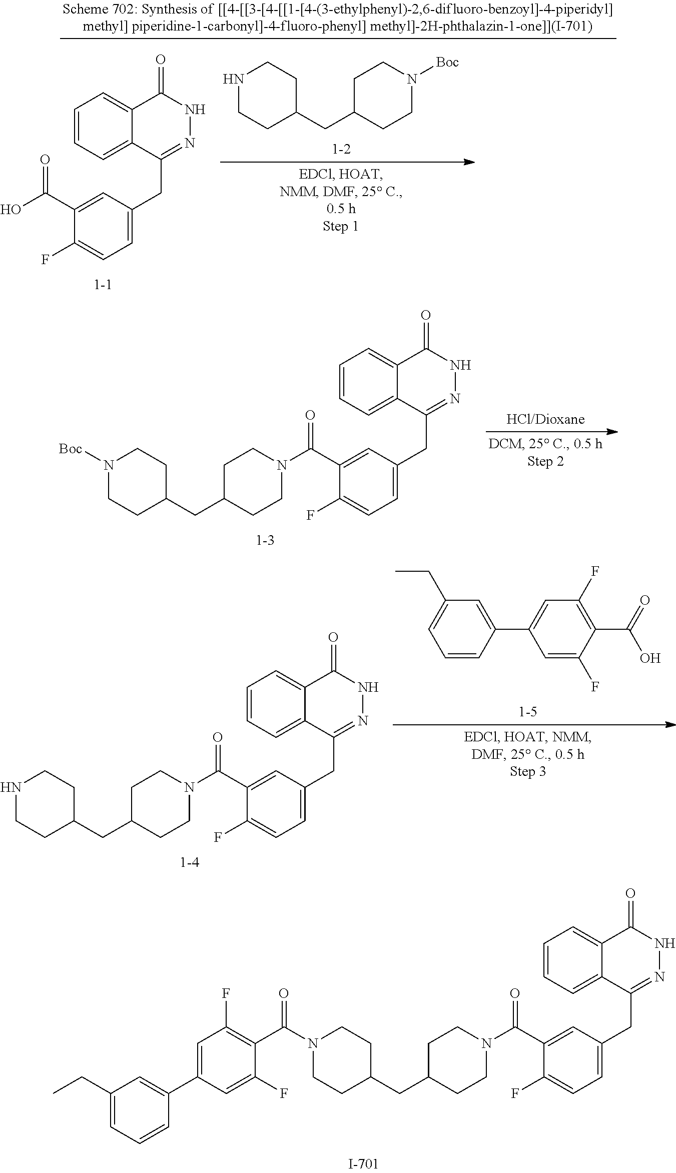 [SMILES: O=C(O)c1cc(Cc2n[nH]c(=O)c3ccccc23)ccc1F ; CC(C)(C)OC(=O)N1CCC(CC2CCNCC2)CC1 ; CC(C)(C)OC(=O)N1CCC(CC2CCN(C(=O)c3cc(Cc4n[nH]c(=O)c5ccccc45)ccc3F)CC2)CC1 ; O=C(c1cc(Cc2n[nH]c(=O)c3ccccc23)ccc1F)N1CCC(CC2CCNCC2)CC1 ; CCc1cccc(-c2cc(F)c(C(=O)O)c(F)c2)c1 ; CCc1cccc(-c2cc(F)c(C(=O)N3CCC(CC4CCN(C(=O)c5cc(Cc6n[nH]c(=O)c7ccccc67)ccc5F)CC4)CC3)c(F)c2)c1]

### Step 1: Synthesis of Intermediate 1-3

To a solution of intermediate 1-1 (200 mg, 670.54 μmol, 1 eq) and intermediate 1-2 (189.37 mg, 670.54 μmol, 1 eq) in DMF (2 mL) was added EDCI (385.63 mg, 2.01 mmol, 3 eq), HOAt (91.27 mg, 670.54 μmol, 93.80 μL, 1 eq) and NMM (339.11 mg, 3.35 mmol, 368.60 μL, 5 eq). The mixture was stirred at 25° C. for 0.5 hr. The reaction mixture was poured into H₂O (5 mL) and extracted with EA (5 mL*3). The combined organic layers were dried over Na₂SO₄, filtered and concentrated under reduced pressure to give a residue. The residue was purified by flash silica gel chromatography (ISCO; 12 g Sepa FlashSilica Flash Column, Eluent of 0˜100% PE/EA@36 mL/min, PE/EA=0:1, Rf=0.5) and the eluent was concentrated under reduced pressure to give a product. Intermediate 1-3 (290 mg, 510.70 μmol, 76.16% yield, 99.089% purity) was obtained as a yellow gum. LCMS (Method D): Rt=0.494 min [M+H]⁺=563.2.

### Step 2: Synthesis of Intermediate 1-4

To a solution of intermediate 1-3 (290 mg, 515.40 μmol, 1 eq) in DCM (3 mL) was added HCl/dioxane (2 M, 3 mL, 11.64 eq). The mixture was stirred at 25° C. for 0.5 hr. The reaction mixture was concentrated under reduced pressure to give a product. The product was used for the next step without purification. Intermediate 1-4 (260 mg, crude, HCl salt) was obtained as a white solid. LCMS (Metho D): Rt=0.304 min [M+H]⁺=463.2.

### Step 3: Synthesis of I-701

To a solution of intermediate 1-4 (100 mg, 200.39 μmol, 1 eq, HCl salt) and intermediate 1-5 (63.06 mg, 240.47 μmol, 1.2 eq) in DMF (1 mL) was added EDCI (115.25 mg, 601.18 gmol, 3 eq), HOAt (27.28 mg, 200.39 μmol, 28.03 μL, 1 eq) and NMM (101.35 mg, 1.00 mmol, 110.16 μL, 5 eq). The mixture was stirred at 25° C. for 0.5 hr. The reaction mixture was poured into H₂O (5 mL) and extracted with EA (2 mL*3). The combined organic layers were dried over Na₂SO₄, filtered and concentrated under reduced pressure to give a residue. The residue was purified by prep-HPLC (column: CD01-Phenomenex luna C18 150*25*10 um; mobile phase: [water (FA)-ACN]; gradient: 55%-85% B over 8 min) and the eluent was concentrated to remove MeCN and then lyophilized to afford I-701 (72.44 mg, 102.49 μmol, 51.14% yield, 100% purity) as a yellow solid. LCMS (Method D): Rt=0.538 min, (M+H)=707.3. ¹H NMR (400 MHz, METHANOL-d4) δ=8.37 (d, J=7.2 Hz, 1H), 7.95-7.88 (m, 1H), 7.88-7.77 (m, 2H), 7.53-7.43 (m, 3H), 7.42-7.34 (m, 3H), 7.32-7.23 (m, 2H), 7.19-7.10 (m, 1H), 4.70-4.67 (m, 1H), 4.63-4.57 (m, 1H), 4.41-4.32 (m, 2H), 3.66-3.56 (m, 1H), 3.44-3.34 (m, 1H), 3.22-3.13 (m, 1H), 3.07-2.97 (m, 1H), 2.93-2.83 (m, 1H), 2.82-2.76 (m, 1H), 2.75-2.69 (m, 2H), 2.02-1.60 (m, 6H), 1.28 (t, J=7.6 Hz, 3H), 1.25-1.06 (m, 6H). ¹⁹F NMR (377 MHz, METHANOL-d4) δ=−115.360, −121.207.

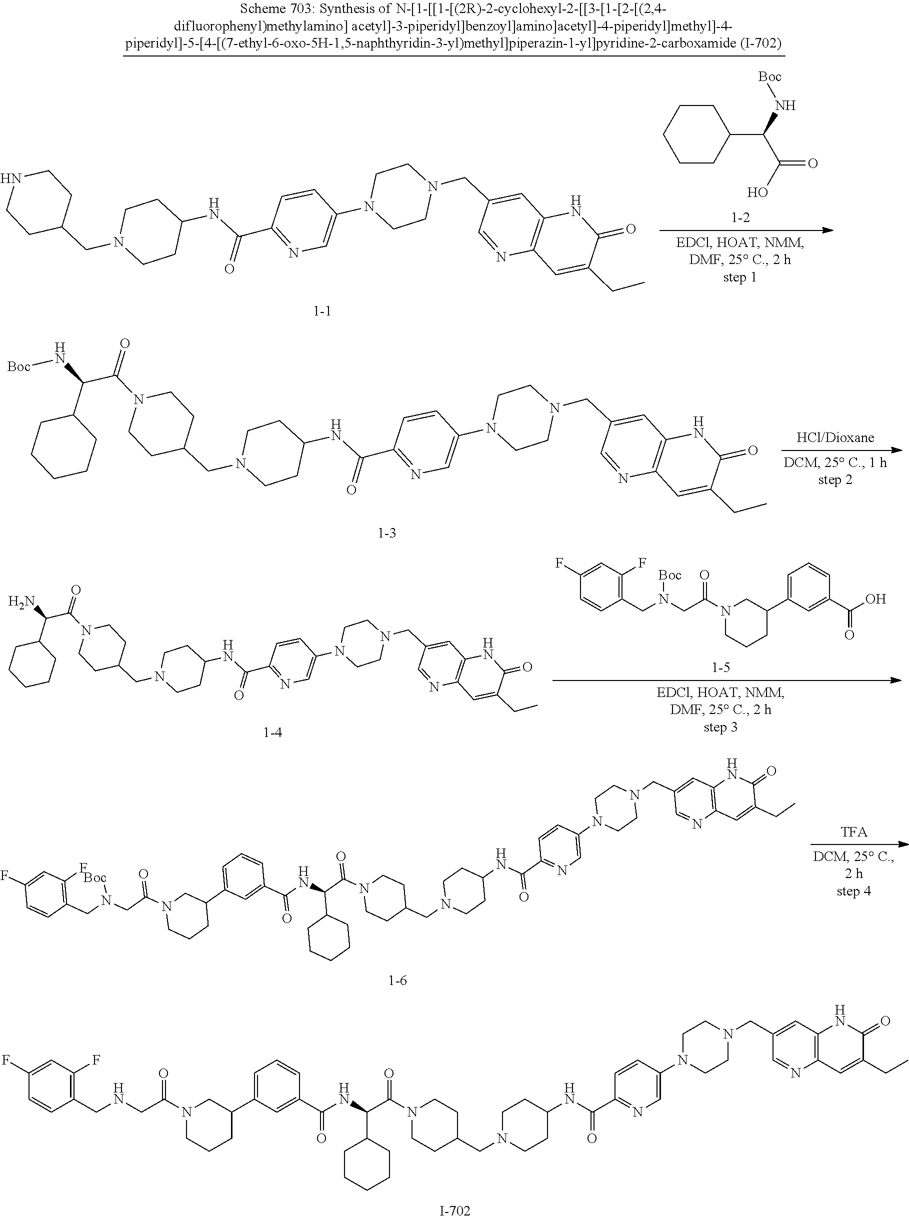

### Step 1: Synthesis of Intermediate 1-3

To a solution of Intermediate 1-2 (190.08 mg, 738.67 μmol, 1.5 eq) in DMF (3 mL) was added EDCI (472.01 mg, 2.46 mmol, 5 eq), NMM (498.09 mg, 4.92 mmol, 541.41 μL, 10 eq), and HOAt (134.05 mg, 984.89 μmol, 137.77 μL, 2 eq) followed by Intermediate 1-1 (300 mg, 492.45 μmol, 1 eq, HCl salt). The mixture was stirred at 25° C. for 1 hr. The reaction mixture was diluted with water (5 mL) and extracted with EA (5 mL*3). The combined organic layers were dried over Nₐ₂SO₄, filtered and concentrated under reduced pressure to give a residue. The residue was purified by flash silica gel chromatography (ISCO; 12 g SepaFlash Silica Flash Column, Eluent of 0˜100% Dichloromethane/Methanol@60 mL/min). The eluent was concentrated under reduced pressure, then lyophilized to afford product. Intermediate 1-3 (250 mg, 292.47 μmol, 59.39% yield, 95% purity) was obtained as a yellow oil. LCMS (Method D): Rt=0.314 min, [M+H]⁺=812.6.

### Step 2: Synthesis of Intermediate 1-4

To a solution of Intermediate 1-3 (200 mg, 246.29 μmol, 1 eq) in DCM (0.5 mL) was added HCl/Dioxane (2 M, 1.5 mL, 12.18 eq). The mixture was stirred at 25° C. for 0.5 hr. The reaction mixture was concentrated under reduced pressure to give a residue. The crude product Intermediate 1-4 (180 mg, 240.51 μmol, 97.65% yield, HCl salt) was obtained as a yellow solid. LCMS (Method D): Rt=0.246 min, [M+H]⁺=712.4.

### Step 3: Synthesis of Intermediate 1-6

To a solution of Intermediate 1-5 (68.62 mg, 140.46 μmol, 1 eq) in DMF (1 mL) was added EDCI (134.63 mg, 702.31 μmol, 5 eq), NMM (142.07 mg, 1.40 mmol, 154.43 μL, 10 eq), and HOAt (38.24 mg, 280.92 μmol, 39.30 μL, 2 eq), followed by Intermediate 1-4 (100 mg, 140.46 μmol, 1 eq). The mixture was stirred at 25° C. for 1 hr. The reaction mixture was diluted with water (5 mL) and extracted with EA (5 mL*3). The combined organic layers were dried over Na₂SO₄, filtered and concentrated under reduced pressure to give a residue. The residue was purified by flash silica gel chromatography (ISCO; 12 g SepaFlash Silica Flash Column, Eluent of 0˜100% Dichloromethane/Methanol@60 mL/min). The eluent was concentrated under reduced pressure then lyophilized to afford product Intermediate 1-6 (70 mg, 59.20 μmol, 42.15% yield) as a yellow oil. LCMS (Method D): Rt=0.467 min, [M+H]⁺=1183.0.

### Step 4: Synthesis of I-702

A solution of Intermediate 1-6 (60 mg, 50.74 μmol, 1 eq) in DCM (0.4 mL) and TFA (0.2 mL) was stirred at 25° C. for 0.5 hr. The reaction mixture was concentrated under reduced pressure to give a residue. The crude product was purified by reversed-phase chromatography (0.1% TFA). The eluent was concentrated under reduced pressure to remove ACN, then lyophilized to afford product. I-702 (30 mg, 24.81 μmol, 48.89% yield, 98.938% purity, TFA salt) was obtained as a white solid. LCMS (Method D): Rt=0.317 min, [M+H]⁺=1082.6. SFC: Rt: 1.745 min, 1.865 min. ¹H NMR (400 MHz, METHANOL-d₄) δ=8.62 (br s, 1H), 8.40-8.33 (m, 1H), 7.98 (br d, J=8.8 Hz, 1H), 7.94 (br s, 1H), 7.88 (s, 1H), 7.81-7.69 (m, 2H), 7.66-7.56 (m, 1H), 7.54-7.41 (m, 3H), 7.22-7.02 (m, 2H), 4.93 (br d, J=7.2 Hz, 1H), 4.67-4.49 (m, 4H), 4.39 (br d, J=10.0 Hz, 1H), 4.32 (br d, J=12.4 Hz, 2H), 4.27-4.08 (m, 3H), 3.77-3.62 (m, 6H), 3.53-3.37 (m, 5H), 3.25 (br d, J=12.4 Hz, 2H), 3.18-2.99 (m, 4H), 2.95-2.73 (m, 3H), 2.71-2.66 (m, 2H), 2.28-2.17 (m, 3H), 2.11-1.98 (m, 3H), 1.90 (br s, 6H), 1.84-1.57 (m, 6H), 1.35-1.22 (m, 7H), 1.21-1.03 (m, 3H). ¹⁹F NMR (377 MHz, METHANOL-d₄) δ=−77.14-108.40-112.84.

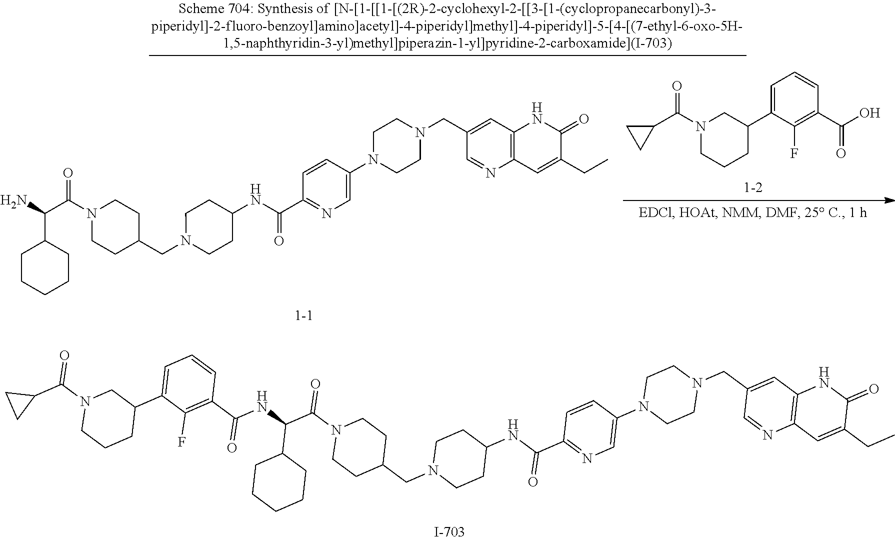

To a solution of Intermediate 1-2 (35 mg, 120.14 μmol, 1 eq) in DMF (1 mL) was added HOAt (16.35 mg, 120.14 μmol, 16.81 μL, 1 eq), EDCI (69.10 mg, 360.43 μmol, 3 eq), NMM (60.76 mg, 600.72 μmol, 66.04 μL, 5 eq) and Intermediate 1-1 (89.92 mg, 120.14 μmol, 1 eq, HCl salt). The mixture was stirred at 25° C. for 1 hr. The reaction mixture was diluted with water (5 mL) and extracted with DCM (5 mL*3). The combined organic layers were dried over Na₂SO₄, filtered and concentrated under reduced pressure to give a residue. The residue was purified by reversed-phase chromatography (0.1% FA condition). The eluent was lyophilized to give product. I-703 (25 mg, 23.98 μmol, 19.96% yield, 94.522% purity) was obtained as a white solid. LCMS (Method D): Retention time: 0.343 min, [M+H]⁺=985.6. SFC: Retention time: 1.605 min, 2.096 min. ¹H NMR (400 MHz, METHANOL-d4) δ=8.50 (d, J=1.6 Hz, 1H), 8.29 (d, J=2.0 Hz, 1H), 7.91 (d, J=9.2 Hz, 1H), 7.84 (s, 1H), 7.77 (s, 1H), 7.61-7.48 (m, 2H), 7.40-7.37 (m, 1H), 7.30-7.21 (m, 1H), 4.97-4.95 (m, 1H), 4.61-4.52 (m, 2H), 4.46-4.23 (m, 2H), 3.99-3.87 (m, 1H), 3.75 (s, 2H), 3.43-3.40 (m, 3H), 3.28-3.19 (m, 2H), 3.14-2.98 (m, 3H), 2.73-2.64 (m, 6H), 2.48-2.31 (m, 3H), 2.08-1.63 (m, 19H), 1.21-1.07 (m, 4H), 0.94-0.74 (m, 5H). ¹⁹F NMR (377 MHz, METHANOL-d₄) δ=−121.6.

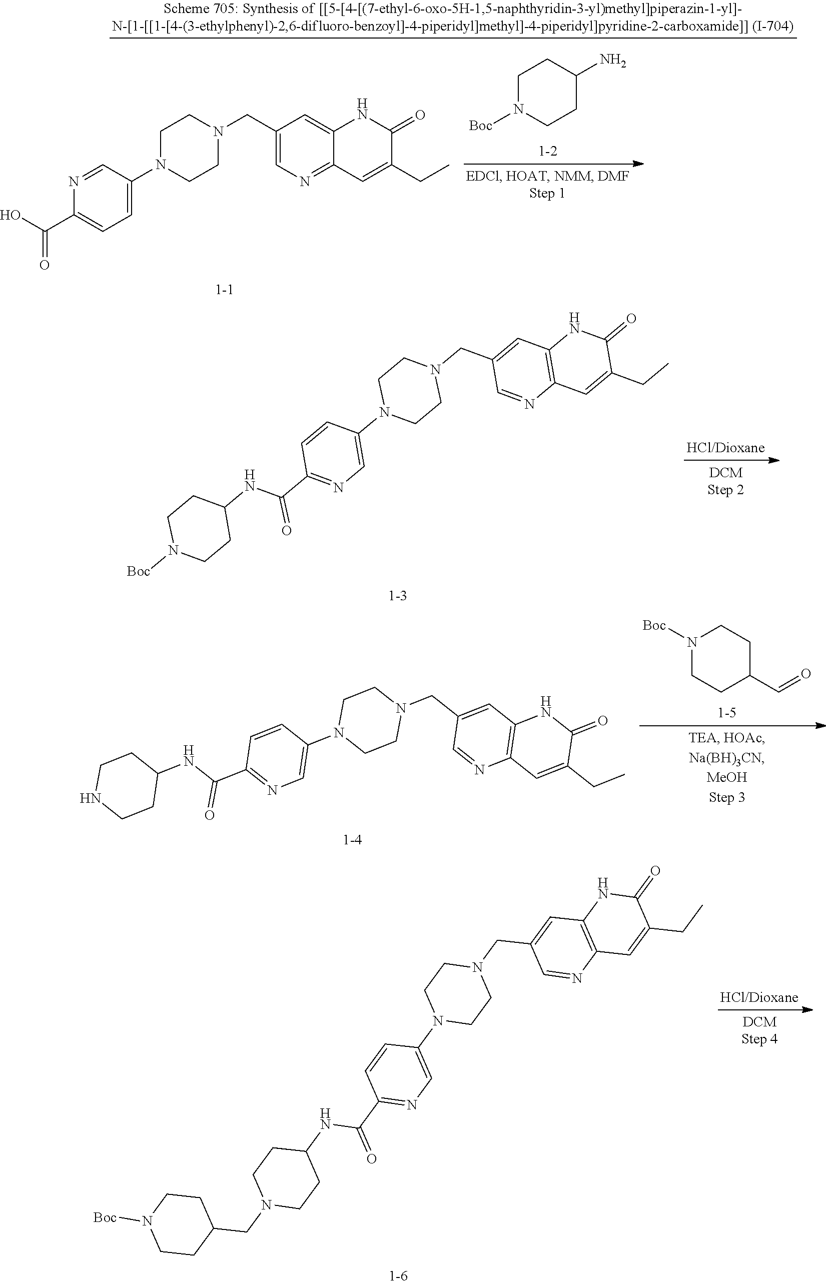 [SMILES: CCc1cc2ncc(CN3CCN(c4ccc(C(=O)O)nc4)CC3)cc2[nH]c1=O ; CC(C)(C)OC(=O)N1CCC(N)CC1 ; CCc1cc2ncc(CN3CCN(c4ccc(C(=O)NC5CCN(C(=O)OC(C)(C)C)CC5)nc4)CC3)cc2[nH]c1=O ; CCc1cc2ncc(CN3CCN(c4ccc(C(=O)NC5CCNCC5)nc4)CC3)cc2[nH]c1=O ; CC(C)(C)OC(=O)N1CCC(C=O)CC1 ; CCc1cc2ncc(CN3CCN(c4ccc(C(=O)NC5CCN(CC6CCN(C(=O)OC(C)(C)C)CC6)CC5)nc4)CC3)cc2[nH]c1=O]

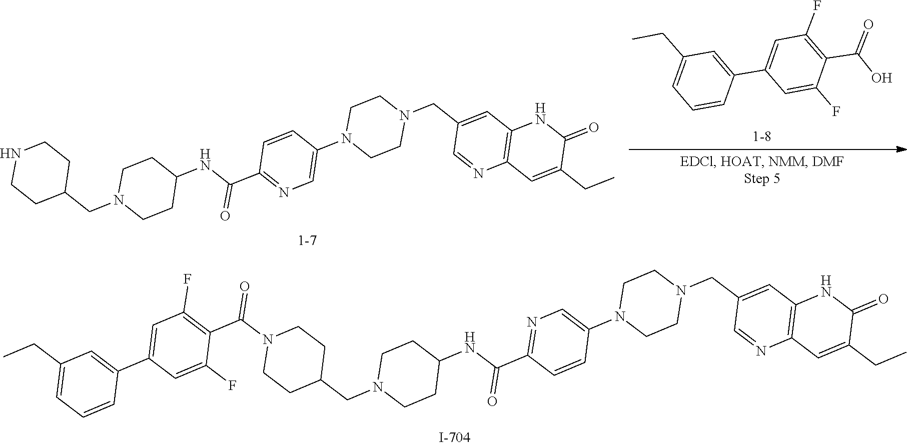

### Step 1: Synthesis of Intermediate 1-3.

To a solution of Intermediate 1-1 (50 mg, 127.08 μmol, 1 eq) and Intermediate 1-2 (30.54 mg, 152.50 μmol, 1.2 eq) in DMF (0.5 mL) was added EDCI (73.09 mg, 381.25 μmol, 3 eq), HOAt (17.30 mg, 127.08 μmol, 17.78 μL, 1 eq) and NMM (64.27 mg, 635.42 μmol, 69.86 μL, 5 eq). The mixture was stirred at 40° C. for 4 hrs. The mixture was diluted with water (5 mL) and extracted with DCM (5 mL*3). The combined organic layer was dried over Na₂SO₄, filtered and concentrated in vacuo to give a residue. The residue was purified by column chromatography (SiO₂, PE:EA=10:1 to DCM:MeOH=0:1), then the organic liquid was concentrated in vacuo to give a residue. Intermediate 1-3 (70 mg, 119.16 μmol, 93.76% yield, 98% purity) was obtained as a yellow oil. LCMS (Method D): Rt=0.324 min, [M+H]⁺=576.4.

### Step 2: Synthesis of Intermediate 1-4.

To a solution of Intermediate 1-3 (70 mg, 121.59 μmol, 1 eq) in DCM (0.5 mL) was added HCl/dioxane (2 M, 0.5 mL, 8.22 eq). The mixture was stirred at 25° C. for 0.5 hr. The reaction mixture was concentrated in vacuo to give a residue. The residue was used in the next step directly without further purification. Intermediate 1-4 (60 mg, HCl salt) was obtained as a white solid. LCMS (Method D): Rt=0.211 min, [M+H]⁺=476.1.

### Step 3: Synthesis of Intermediate 1-6.

To a solution of Intermediate 1-4 (60 mg, 117.18 μmol, 1 eq, HCl salt) in MeOH (1 mL) was added dropwise TEA (47.43 mg, 468.71 μmol, 65.24 μL, 4 eq) at 25° C. for 10 min. Then Intermediate 1-5 (37.49 mg, 175.77 μmol, 1.5 eq) and HOAc (28.15 mg, 468.71 μmol, 26.83 μL, 4 eq) as added at 25° C. for 1 h. After addition, NaBH₃CN (44.18 mg, 703.06 μmol, 6 eq) was added. The resulting mixture was stirred at 25° C. for 1 hr. The mixture was diluted with water (5 mL) and extracted with DCM (5 mL*3). The combined organic layer was dried over Na₂SO₄, filtered and concentrated in vacuo to give a residue. The residue was purified by column chromatography (SiO₂, PE:EA=10:1 to EA:MeOH=0:1), then the organic liquid was concentrated in vacuo to give a residue. Intermediate 1-6 (40 mg, 58.26 μmol, 49.72% yield, 98% purity) was obtained as a white solid. LCMS (Method D): Rt=0.286 min, [M+H]⁺=673.5.

### Step 4: Synthesis of Intermediate 1-7.

To a solution of Intermediate 1-6 (30 mg, 44.59 μmol, 1 eq) in DCM (0.1 mL) was added HCl/dioxane (2 M, 0.2 mL, 8.97 eq). The mixture was stirred at 25° C. for 0.5 hr. The reaction mixture was concentrated in vacuo to give a residue. The residue was used in the next step directly without further purification. Intermediate 1-7 (40 mg, crude, HCl salt) was obtained as a white solid. LCMS (Method D): Rt=0.179 min, [M+H]⁺=573.3.

### Step 5: Synthesis of I-704.

To a solution of Intermediate 1-7 (40 mg, 65.66 μmol, 1 eq, HCl salt) and Intermediate 1-8 (18.94 mg, 72.23 μmol, 1.1 eq) in DMF (0.5 mL) was added EDCI (37.76 mg, 196.98 μmol, 3 eq), HOAt (8.94 mg, 65.66 μmol, 9.18 μL, 1 eq) and NMM (33.21 mg, 328.30 μmol, 36.09 μL, 5 eq). The mixture was stirred at 25° C. for 1 hr. The mixture was diluted with water (5 mL) and extracted with DCM (5 mL*3). The combined organic layer was dried over Na₂SO₄, filtered and concentrated in vacuo to give a residue. The residue was purified by prep-HPLC (column: CD02-Waters Xbidge BEH C18 150*25*10 um; mobile phase: [water (NH₄HCO₃)-ACN]; gradient: 46%-76% B over 10 min). Then the eluent was concentrated in vacuo and lyophilized. I-704 (21.25 mg, 24.38 μmol, 37.14% yield, 99.030% purity, FA salt) was obtained as a white solid. LCMS (Method D): Rt=0.356 min, [M+H]⁺=817.3. ¹H NMR (400 MHz, DMSO-d6) δ =11.87 (s, 1H), 8.41 (s, 1H), 8.28 (d, J=2.4 Hz, 1H), 8.16-8.14 (m, 1H), 7.83 (d, J=8.4 Hz, 1H), 7.76 (s, 1H), 7.63 (s, 2H), 7.61-7.56 (m, 3H), 7.43-7.38 (m, 2H), 7.37-7.29 (m, 2H), 4.52 (d, J=14.4 Hz, 1H), 3.79-3.69 (m, 1H), 3.66 (s, 2H), 3.48 (d, J=12.4 Hz, 1H), 3.19-3.07 (m, 2H), 2.91-2.76 (m, 3H), 2.71-2.66 (m, 3H), 2.58-2.55 (m, 4H), 2.54 (s, 1H), 2.20-2.10 (m, 2H), 2.03-1.95 (m, 2H), 1.88-1.82 (m, 2H), 1.77-1.71 (m, 3H), 1.65-1.58 (m, 2H), 1.31-1.28 (m, 1H), 1.26-1.16 (m, 8H), 1.08 (s, 2H). ¹⁹F NMR (376 MHz, DMSO-d6) δ=−114.038.

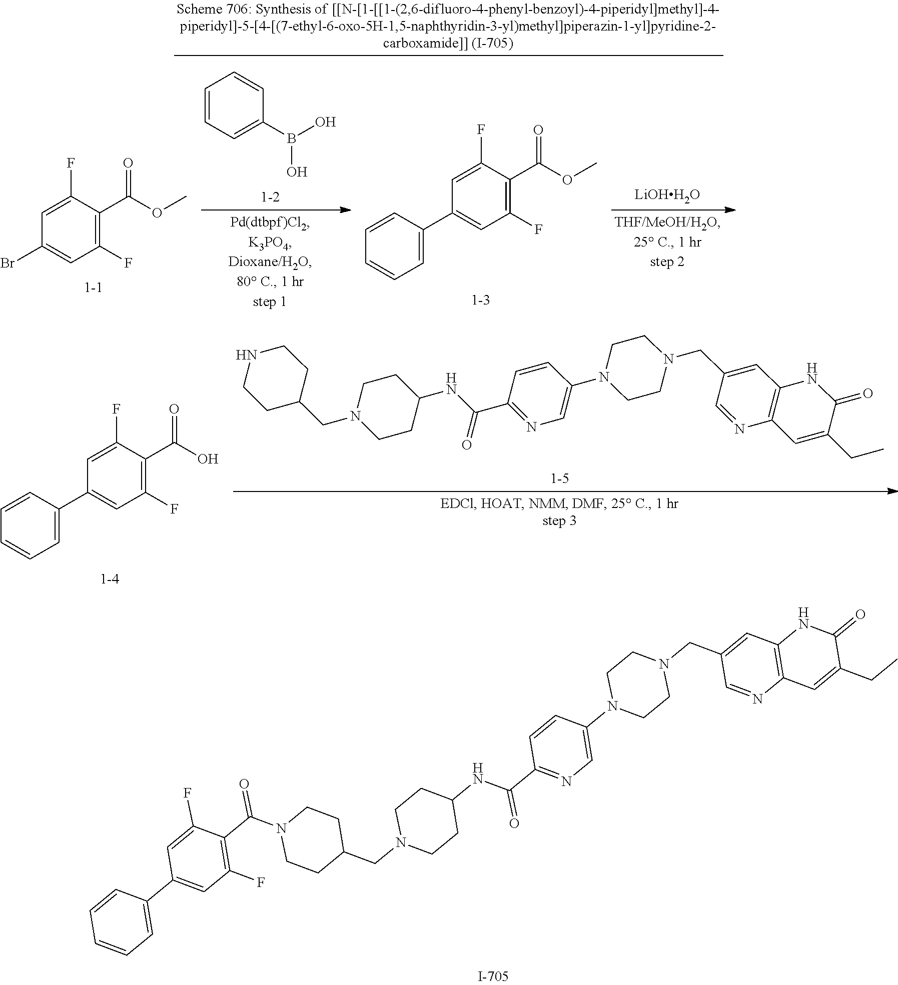

### Step 1: Synthesis of Intermediate 1-3

A mixture of Intermediate 1-1 (5 g, 19.92 mmol, 1 eq), Intermediate 1-2 (2.67 g, 21.91 mmol, 1.1 eq), Pd(dtbpf)Cl₂ (1.30 g, 1.99 mmol, 0.1 eq) and K₃PO₄ (12.68 g, 59.76 mmol, 3 eq) in dioxane (50 mL) and H₂O (10 mL) was degassed and purged with N₂ 3 times, and then the mixture was stirred at 80° C. for 1 hr under N₂ atmosphere. The reaction was concentrated under reduced pressure and poured into H₂O (10 mL). The mixture was extracted with EA (20 mL*3). The combined organic layers were dried over Na₂SO₄, filtered and concentrated under reduced pressure to give a residue. The residue was purified by flash silica gel chromatography (ISCO; 80 g SepaFlash Silica Flash Column, Eluent of 0˜7% Ethyl acetate/Petroleum ether gradient @100 mL/min) and concentrated under reduced pressure to give Intermediate 1-3 (4.95 g, 18.55 mmol, 93.11% yield, 93% purity) as a yellow solid. LCMS (Method D): Retention time: 0.471 min, [M+H]⁺=249.0.

### Step 2: Synthesis of Intermediate 1-4

To a solution of Intermediate 1-3 (4.7 g, 18.93 mmol, 1 eq) in THF (20 mL), MeOH (20 mL) and H₂O (7 mL) was added LiOH H₂O (3.97 g, 94.67 mmol, 5 eq). The mixture was stirred at 25° C. for 1 hr. The mixture was concentrated under reduced pressure and adjusted to pH=7 with HCl (1 M). The mixture was extracted with EA (20 mL*3). The organic layers were dried over Na₂SO₄, filtered and concentrated under reduced pressure to give Intermediate 1-4 (4.34 g, 18.35 mmol, 96.89% yield, 99% purity) as a white solid. LCMS (Method D): Retention time: 0.397 min, [M+H]⁺=235.0. ¹H NMR (400 MHz, METHANOL-d4) δ=7.67-7.63 (m, 2H), 7.50-7.44 (m, 2H), 7.43-7.38 (m, 1H), 7.31-7.25 (m, 2H). ¹⁹F NMR (377 MHz, METHANOL-d₄) δ=−113.850.

### Step 3: Synthesis of I-705

To a solution of Intermediate 1-4 (40 mg, 170.80 μmol, 1 eq) and Intermediate 1-5 (104.05 mg, 170.80 μmol, 1 eq, HCl salt) in DMF (1 mL) was added EDCI (98.23 mg, 512.39 μmol, 3 eq), HOAt (23.25 mg, 170.80 μmol, 23.89 μL, 1 eq) and NMM (86.38 mg, 853.98 μmol, 93.89 μL, 5 eq). The mixture was stirred at 25° C. for 1 hr. The reaction mixture was concentrated in vacuum to give a residue. The residue was purified by reversed phase chromatography (0.1% FA) and lyophilized to give I-705 (8.38 mg, 9.77 μmol, 5.72% yield, 97.337% purity, FA salt) as a white solid. LCMS (Method D): Retention time: 0.304 min, [M+H]⁺=789.5. ¹H NMR (400 MHz, METHANOL-d4) δ=8.58 (d, J=1.6 Hz, 1H), 8.36-8.31 (m, 1H), 7.95 (d, J=8.8 Hz, 1H), 7.91 (s, 1H), 7.86 (s, 1H), 7.69-7.63 (m, 2H), 7.52-7.41 (m, 4H), 7.41-7.36 (m, 2H), 4.75 (d, J=13.6 Hz, 1H), 4.34 (s, 2H), 4.23-4.11 (m, 1H), 3.68 (d, J=10.4 Hz, 3H), 3.60 (s, 4H), 3.29-3.16 (m, 7H), 3.13 (d, J=6.4 Hz, 2H), 3.02-2.92 (m, 1H), 2.71-2.62 (m, 2H), 2.31-2.17 (m, 3H), 2.10-1.96 (m, 3H), 1.87 (d, J=12.8 Hz, 1H), 1.43-1.30 (m, 2H), 1.28 (t, J=7.2 Hz, 3H). ¹⁹F NMR (377 MHz, METHANOL-d₄) δ=−115.143.

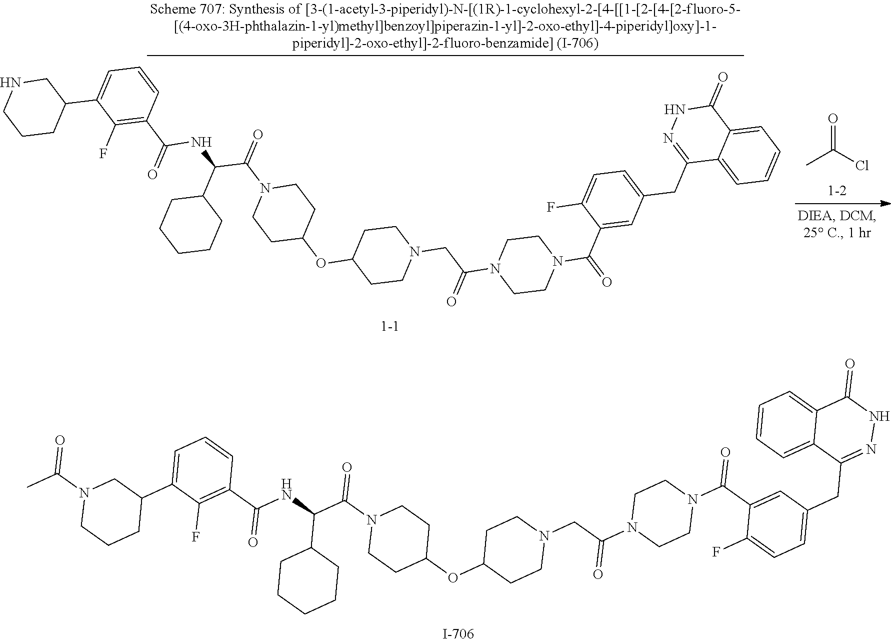

### Step 1: Synthesis of I-706

To a mixture of intermediate 1-1 (400 mg, 411.70 μmol, 1 eq, HCl salt) in DCM (4 mL) was added intermediate 1-2 (48.48 mg, 617.56 μmol, 43.91 μL, 1.5 eq) and DIEA (106.42 mg, 823.41 μmol, 143.42 L, 2 eq) at 25° C., then the mixture was stirred at 25° C. for 0.5 h. The reaction mixture was concentrated to give a residue. The residue was purified by prep-HPLC (column: CD05-Phenomenex luna C18 150*40*10 um; mobile phase: [water (FA)-ACN]; gradient: 10%-40% B over 11 min) and lyophilized to give the product. I-706 (212.86 mg, 207.33 μmol, 50.36% yield, 99.66% purity, FA salt) was obtained as a white solid. LCMS (Method D): Retention time: 0.373 min, [M/2+H]⁺=489.5. SFC: Retention time: 5.853 min, 7.507 min. ¹H NMR (400 MHz, METHANOL-d4) δ=8.52-8.39 (m, 1H), 8.37 (d, J=7.6 Hz, 1H), 7.95 (d, J=6.8 Hz, 1H), 7.91-7.79 (m, 2H), 7.62-7.45 (m, 3H), 7.37 (t, J=6.4 Hz, 1H), 7.30-7.21 (m, 1H), 7.17 (t, J=8.8 Hz, 1H), 4.96 (t, J=6.8 Hz, 1H), 4.60 (d, J=11.6 Hz, 1H), 4.39 (s, 2H), 4.10-3.87 (m, 3H), 3.80 (s, 3H), 3.77-3.66 (m, 4H), 3.62 (s, 2H), 3.56-3.43 (m, 3H), 3.40-3.32 (m, 2H), 3.28-3.21 (m, 1H), 3.19 (s, 1H), 3.14-3.10 (m, 1H), 3.09-2.99 (m, 1H), 2.91-2.76 (m, 2H), 2.76-2.62 (m, 1H), 2.13 (d, J=8.8 Hz, 3H), 2.00 (s, 4H), 1.86 (d, J=11.2 Hz, 5H), 1.83-1.73 (m, 4H), 1.73-1.60 (m, 4H), 1.59-1.48 (m, 1H), 1.36-1.21 (m, 3H), 1.20-1.08 (m, 2H). ¹⁹F NMR (376 MHz, METHANOL-d4)

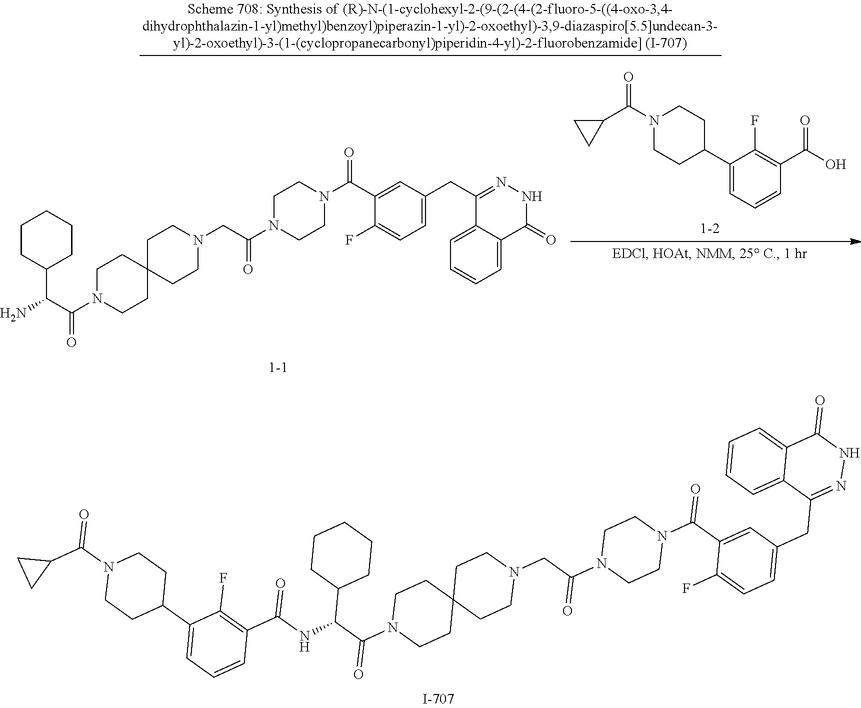 [SMILES: N[C@@H](C(=O)N1CCC2(CCN(CC(=O)N3CCN(C(=O)c4cc(Cc5n[nH]c(=O)c6ccccc56)ccc4F)CC3)CC2)CC1)C1CCCCC1 ; O=C(O)c1cccc(C2CCN(C(=O)C3CC3)CC2)c1F ; O=C(N[C@@H](C(=O)N1CCC2(CCN(CC(=O)N3CCN(C(=O)c4cc(Cc5n[nH]c(=O)c6ccccc56)ccc4F)CC3)CC2)CC1)C1CCCCC1)c1cccc(C2CCN(C(=O)C3CC3)CC2)c1F]

To a solution of Intermediate 1-1 (500 mg, 679.06 μmol, 1 eq, HCl salt) and Intermediate 1-2 (197.82 mg, 679.06 μmol, 1 eq) in DMF (5 mL) was added EDCI (260.35 mg, 1.36 mmol, 2 eq), HOAt (92.43 mg, 679.06 μmol, 94.99 μL, 1 eq) and NMM (343.42 mg, 3.40 mmol, 373.29 μL, 5 eq). The mixture was stirred at 25° C. for 1 hr. To the reaction mixture was added into H₂O (10 mL) and then it was extracted with DCM (10 mL*3), and the combined organic phase was washed, dried by Na₂SO₄, filtered and concentrated. The residue was purified by prep-HPLC (column: Phenomenex luna C18 150*40 mm*15 um; mobile phase: [water (FA)-ACN]; gradient: 17%-47% B over 10 min) and the eluent was concentrated to remove ACN and lyophilized to give product. I-707 (330.25 mg, 320.35 μmol, 47.18% yield, 98.864% purity, FA salt) was obtained as a yellow solid. LCMS (Method D): Retention time: 0.303 min, [M+H]⁺=990.7. SFC: Retention time: 0.438 min, 0.921 min. ¹H NMR (400 MHz, METHANOL-d4) δ=8.51 (s, 1H), 8.39 (d, J=7.6 Hz, 1H), 8.02-7.94 (m, 1H), 7.93-7.80 (m, 2H), 7.62-7.45 (m, 3H), 7.40 (t, J=6.8 Hz, 1H), 7.25 (t, J=7.6 Hz, 1H), 7.19 (t, J=8.8 Hz, 1H), 4.98 (d, J=7.2 Hz, 1H), 4.70 (d, J=13.2 Hz, 1H), 4.52 (d, J=14.0 Hz, 1H), 4.41 (s, 2H), 3.88-3.74 (m, 4H), 3.66 (d, J=19.2 Hz, 4H), 3.59-3.45 (m, 4H), 3.36 (d, J=3.2 Hz, 2H), 3.32-3.22 (m, 2H), 2.79 (d, J=12.8 Hz, 5H), 2.07-1.96 (m, 2H), 1.92-1.60 (m, 15H), 1.54 (d, J=4.0 Hz, 2H), 1.39-1.22 (m, 3H), 1.16 (d, J=6.8 Hz, 2H), 0.96-0.88 (m, 2H), 0.88-0.80 (m, 2H). ¹⁹F NMR (377 MHz, METHANOL-d₄), δ=−120.72, −122.41.

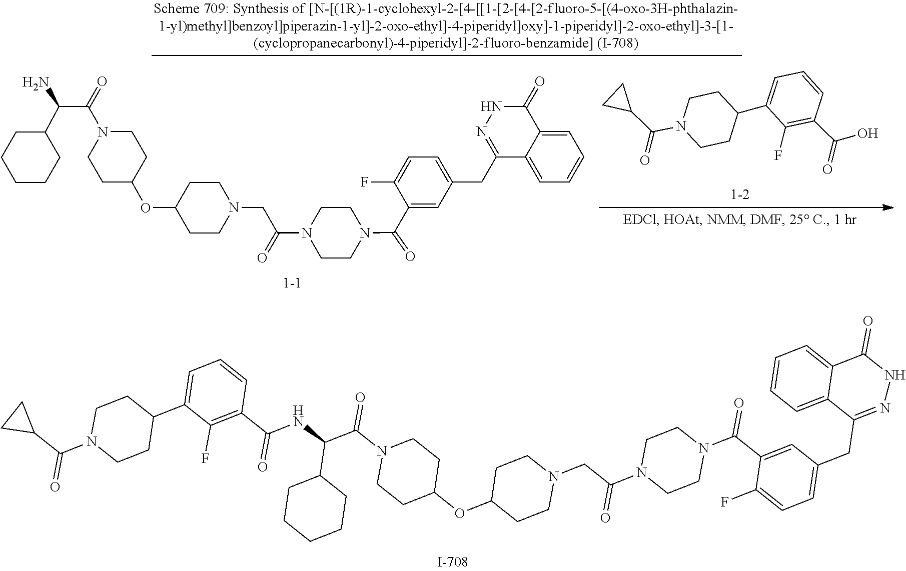

To a mixture of intermediate 1-1 (500 mg, 652.45 μmol, 1 eq, HCl salt) in DMF (5 mL) was added intermediate 1-2 (190.07 mg, 652.45 μmol, 1 eq), EDCI (375.23 mg, 1.96 mmol, 3 eq), HOAt (88.81 mg, 652.45 μmol, 91.27 μL, 1 eq) and NMM (329.97 mg, 3.26 mmol, 358.66 μL, 5 eq) at 25° C., then the mixture was stirred at 25° C. for 1 h. The reaction mixture was diluted with H₂O (5 mL) and extracted with DCM (5 mL×3), and the combined organic layers were dried over Na₂SO₄, filtered and concentrated to give a residue. The residue was purified by prep-HPLC (column: Phenomenex luna C18 150*40 mm*15 um; mobile phase: [water (FA)-ACN]; gradient: 18%-48% B over 10 min) and lyophilized to give product. I-708 (214.19 mg, 213.34 μmol, 32.70% yield, 99.92% purity) was obtained as a white solid. LCMS (Method D): Retention time: 0.385 min, [M+H]⁺=1003.6. SFC: Retention time: 1.455 min. ¹H NMR (400 MHz, METHANOL-d4) δ=8.37 (d, J=7.6 Hz, 1H), 7.98-7.93 (m, 1H), 7.91-7.80 (m, 2H), 7.54 (t, J=6.8 Hz, 1H), 7.51-7.43 (m, 2H), 7.38 (d, J=4.8 Hz, 1H), 7.23 (t, J=7.6 Hz, 1H), 7.16 (t, J=8.8 Hz, 1H), 4.97 (d, J=7.6 Hz, 1H), 4.69 (d, J=12.4 Hz, 1H), 4.50 (d, J=14.0 Hz, 1H), 4.39 (s, 2H), 4.09-3.98 (m, 1H), 3.97-3.83 (m, 1H), 3.82-3.71 (m, 4H), 3.67 (s, 1H), 3.63-3.41 (m, 5H), 3.34 (d, J=2.0 Hz, 2H), 3.29-3.12 (m, 4H), 2.84-2.71 (m, 3H), 2.34-2.19 (m, 2H), 2.06-1.97 (m, 2H), 1.95 (s, 1H), 1.92-1.80 (m, 7H), 1.80-1.74 (m, 2H), 1.70 (d, J=11.6 Hz, 3H), 1.65-1.49 (m, 4H), 1.36-1.21 (m, 3H), 1.20-1.08 (m, 2H), 0.93-0.79 (m, 4H). ¹⁹F NMR (376 MHz, METHANOL-d4) δ=−120.8, −122.4.

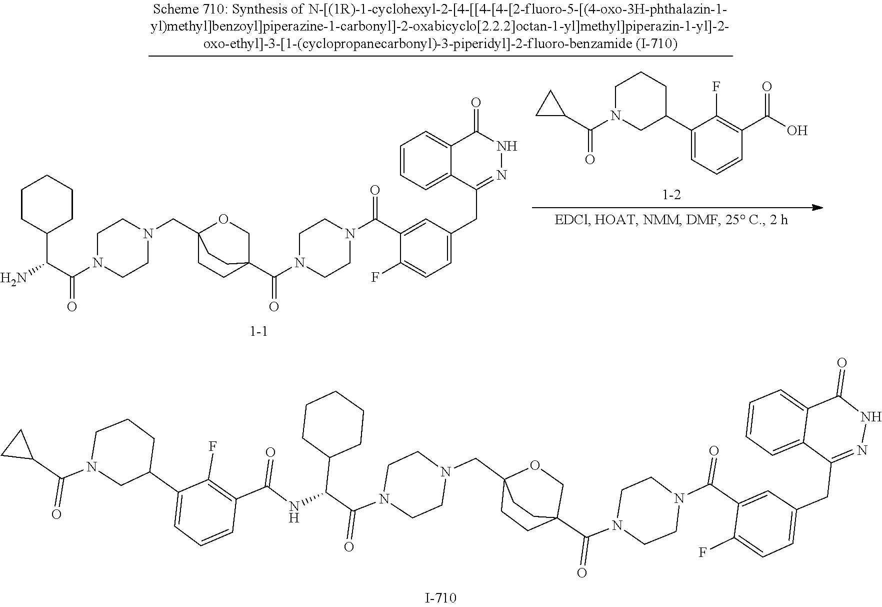 [SMILES: N[C@@H](C(=O)N1CCN(CC23CCC(C(=O)N4CCN(C(=O)c5cc(Cc6n[nH]c(=O)c7ccccc67)ccc5F)CC4)(CC2)CO3)CC1)C1CCCCC1 ; O=C(O)c1cccc(C2CCCN(C(=O)C3CC3)C2)c1F ; O=C(N[C@@H](C(=O)N1CCN(CC23CCC(C(=O)N4CCN(C(=O)c5cc(Cc6n[nH]c(=O)c7ccccc67)ccc5F)CC4)(CC2)CO3)CC1)C1CCCCC1)c1cccc(C2CCCN(C(=O)C3CC3)C2)c1F]

To a solution of Intermediate 1-2 (90 mg, 308.94 μmol, 1 eq) in DMF (1 mL) was added EDCI (296.12 mg, 1.54 mmol, 5 eq), NMM (312.49 mg, 3.09 mmol, 339.66 μL, 10 eq) and HOAt (84.10 mg, 617.88 μmol, 86.43 μL, 2 eq), followed by Intermediate 1-1 (264.51 mg, 339.84 μmol, 1.1 eq, HCl salt). The mixture was stirred at 25° C. for 2 hr. The residue was diluted with H₂O (5 mL) and extracted with EA (5 mL*3), dried over Na₂SO₄, filtered and concentrated under reduced pressure to give a residue. The residue was purified by flash silica gel chromatography (ISCO; 12 g SepaFlash Silica Flash Column, Eluent of 0˜100% Dichloromethane/Methanol@60 mL/min). The eluent was concentrated under reduced pressure then lyophilized to give I-710 (212 mg, 208.28 μmol, 67.42% yield, 99.74% purity) as a white solid. LCMS (Method D): Rt=0.409 min, [M+H]⁺=1015.7. ¹H NMR (400 MHz, METHANOL-d4) δ=8.38-8.36 (m, 1H), 7.98-7.92 (m, 1H), 7.91-7.79 (m, 2H), 7.62-7.44 (m, 3H), 7.38-7.36 (m, 1H), 7.28-7.22 (m, 1H), 7.16 (t, J=8.8 Hz, 1H), 4.98-4.90 (m, 1H), 4.67-4.51 (m, 1H), 4.46-4.35 (m, 3H), 3.95 (s, 2H), 3.75 (br s, 5H), 3.71-3.62 (m, 2H), 3.58 (br d, J=4.0 Hz, 3H), 3.26-2.96 (m, 2H), 2.89-2.68 (m, 1H), 2.66-2.47 (m, 4H), 2.31 (s, 2H), 2.24-2.12 (m, 2H), 2.08-1.61 (m, 18H), 1.39-1.02 (m, 6H), 0.94-0.77 (m, 4H). ¹⁹F NMR (377 MHz, METHANOL-d4) δ=−120.70-121.47.

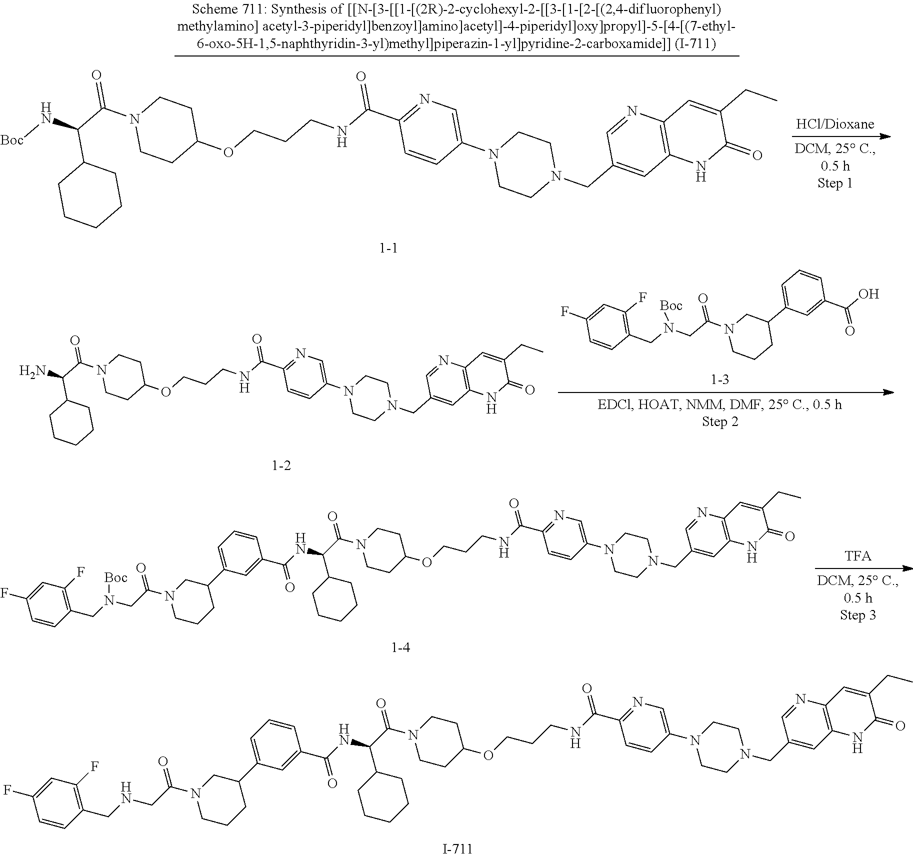

### Step 1: Synthesis of Intermediate 1-2

To a solution of intermediate 1-1 (190 mg, 245.80 μmol, 1 eq) in DCM (2 mL) was added HCl/dioxane (2 M, 2 mL, 16.27 eq). The mixture was stirred at 25° C. for 0.5 hr. The reaction mixture was concentrated under reduced pressure to give a product. The product was used for the next step without purification. LCMS (Method D): Rt=0.290 min, [M+H]⁺=673.4.

### Step 2: Synthesis of Intermediate 1-4

To a solution of intermediate 1-2 (120 mg, 169.18 μmol, 1 eq, HCl salt) and intermediate 1-3 (99.18 mg, 203.01 μmol, 1.2 eq) in DMF (1.2 mL) was added EDCI (97.29 mg, 507.53 μmol, 3 eq), HOAt (23.03 mg, 169.18 μmol, 23.67 μL, 1 eq) and NMM (85.56 mg, 845.88 μmol, 93.00 μL, 5 eq). The mixture was stirred at 25° C. for 0.5 hr. The reaction mixture was poured into H₂O (5 mL) and extracted with EA (2 mL*3). The combined organic layers were dried over Na₂SO₄, filtered and concentrated under reduced pressure to give a residue. The residue was purified by flash silica gel chromatography (ISCO; 12 g SepaFlashSilica Flash Column, Eluent of 0˜100% DCM/MeOH@36 mL/min, DCM/MeOH=10:1, Rf=0.4) and the eluent was concentrated under reduced pressure to give Intermediate 1-4 (160 mg, 131.30 μmol, 77.61% yield, 93.83% purity) as a white gum. LCMS (Method D): Rt=0.444 min, [M+H]⁺=1143.7.

### Step 3: Synthesis of I-711

To a solution of intermediate 1-4 (140 mg, 122.45 μmol, 1 eq) in DCM (1.4 mL) was added TFA (429.80 mg, 3.77 mmol, 280.00 μL, 30.78 eq). The mixture was stirred at 25° C. for 0.5 hr. The reaction mixture was concentrated under reduced pressure to give a residue. The residue was purified by prep-HPLC (column: CD06-Waters Xbidge C18 150*40*10 um; mobile phase: [water (TFA)-ACN]; gradient: 18%-48% B over 11 min) and the eluent was concentrated to remove MeCN and then lyophilized to afford I-711 (34.19 mg, 29.32 μmol, 23.95% yield, 99.26% purity, TFA salt) as a yellow solid. LCMS (Method D): Rt=0.350 min, [M+H]⁺=1043.9. SFC: Rt=2.978 min, 3.676 min. ¹H NMR (400 MHz, METHANOL-d4) δ=8.66-8.54 (m, 1H), 8.33 (s, 1H), 8.03-7.83 (m, 3H), 7.77-7.58 (m, 3H), 7.55-7.33 (m, 3H), 7.22-6.98 (m, 2H), 4.92 (d, J=5.2 Hz, 1H), 4.64-4.42 (m, 3H), 4.35-4.29 (m, 2H), 4.29-4.13 (m, 2H), 4.10 (d, J=16.0 Hz, 1H), 4.02-3.77 (m, 2H), 3.67 (s, 8H), 3.57-3.35 (m, 7H), 3.26-3.13 (m, 1H), 3.06-2.73 (m, 2H), 2.71-2.65 (m, 2H), 2.10-1.82 (m, 9H), 1.80-1.60 (m, 7H), 1.31-1.27 (m, 4H), 1.26-1.04 (m, 4H). ¹⁹F NMR (377 MHz, METHANOL-d4) δ=−108.22 (1 F), −112.83 (1 F).

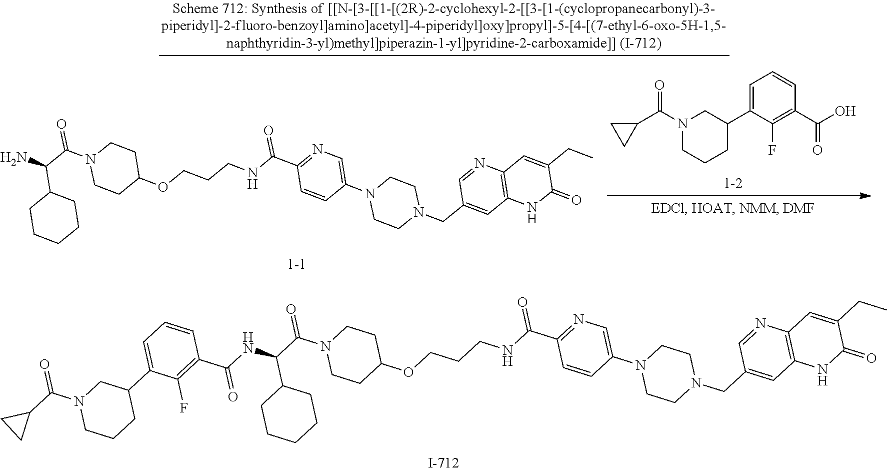

To a solution of Intermediate 1-2 (25 mg, 85.82 μmol, 1 eq) and Intermediate 1-1 (66.96 mg, 94.40 μmol, 1.1 eq, HCl salt) in DMF (0.5 mL) was added EDCI (49.35 mg, 257.45 μmol, 3 eq), HOAt (11.68 mg, 85.82 μmol, 12.00 μL, 1 eq), and NMM (43.40 mg, 429.09 μmol, 47.17 μL, 5 eq). The mixture was stirred at 25° C. for 0.5 hr. The mixture was diluted with water (5 mL) and extracted with DCM (5 mL*3). The combined organic layer was dried over Na₂SO₄, filtered and concentrated in vacuo to give a residue. The residue was purified by reverse phase chromatography (0.1% FA condition), then the eluent was concentrated in vacuo and lyophilized to give I-712 (43.69 mg, 45.91 μmol, 53.50% yield, 99.43% purity) as a white solid. LCMS (Method H): Rt=0.679 min, [M+H]⁺=946.5. SFC: Retention time: 2.835 min, 3.911 min. ¹H NMR (400 MHz, METHANOL-d4) δ=8.47 (d, J=12.8 Hz, 1H), 8.23 (s, 1H), 7.90-7.87 (m, 1H), 7.83 (d, J=2.4 Hz, 1H), 7.75 (d, J=11.2 Hz, 1H), 7.60-7.39 (m, 2H), 7.39-7.30 (m, 1H), 7.28-7.12 (m, 1H), 4.96 (s, 1H), 4.57 (d, J=7.2 Hz, 1H), 4.38 (d, J=9.6 Hz, 1H), 4.09-3.90 (m, 1H), 3.89-3.75 (m, 1H), 3.74 (s, 2H), 3.70-3.64 (m, 4H), 3.62-3.56 (m, 1H), 3.55-3.46 (m, 2H), 3.41 (s, 4H), 3.27-3.17 (m, 1H), 3.16-2.98 (m, 1H), 2.95-2.72 (m, 1H), 2.70-2.63 (m, 4H), 2.61-2.53 (m, 2H), 2.01 (s, 1H), 1.99-1.86 (m, 6H), 1.83 (d, J=13.6 Hz, 4H), 1.77-1.65 (m, 4H), 1.62 (d, J=9.2 Hz, 2H), 1.30-1.26 (m, 4H), 1.24-1.15 (m, 2H), 1.12 (d, J=12.0 Hz, 2H), 0.93-0.75 (m, 4H). ¹⁹F NMR (376 MHz, METHANOL-d₄) δ=−121.498.

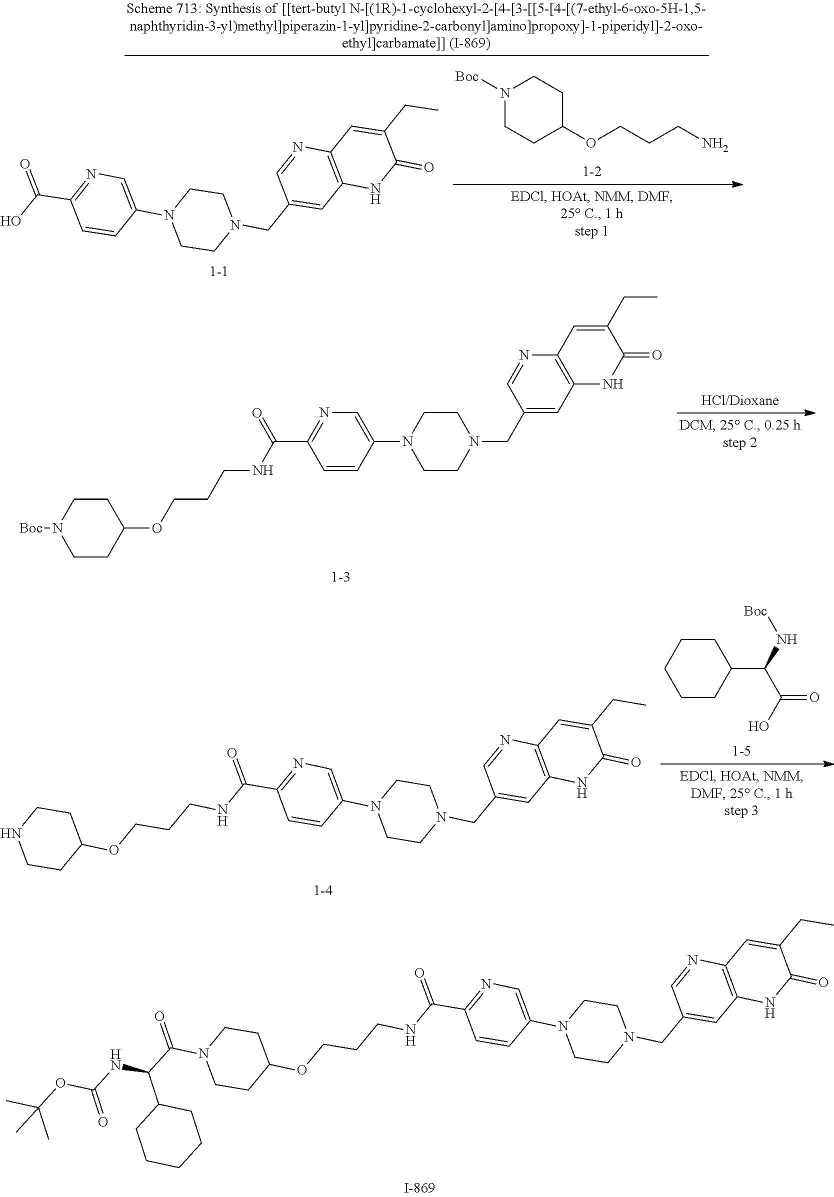

### Step 1: Synthesis of Intermediate 1-3

To a solution of Intermediate 1-1 (480 mg, 1.22 mmol, 1 eq) in DMF (5 mL) was added EDCI (701.63 mg, 3.66 mmol, 3 eq), HOAt (166.06 mg, 1.22 mmol, 170.67 μL, 1 eq), and NMM (617.00 mg, 6.10 mmol, 670.66 μL, 5 eq), and the mixture was stirred at 25° C. for 0.5 hr, and then Intermediate 1-2 (315.20 mg, 1.22 mmol, 1 eq) was added to the mixture. The resulting mixture was stirred at 25° C. for 1 hr. The reaction was poured into water (10 mL) and extracted with EA (5 mL*3). The combined organic layers were dried over Na₂SO₄, filtered and concentrated under reduced pressure to give a residue. The residue was purified by flash silica gel chromatography (ISCO; 12 g SepaFlash Silica Flash Column, Eluent of 0˜100% DCM/MeOH@18 mL/min, DCM/MeOH=10:1, Rf=0.6) and the eluent was concentrated under reduced pressure to give Intermediate 1-3 (400 mg, 605.32 μmol, 49.62% yield, 95.91% purity) as a red solid. LCMS (Method D): Retention time: 0.359 min, [M+H]⁺=634.3.

### Step 2: Synthesis of Intermediate 1-4.

To a solution of Intermediate 1-3 (330 mg, 520.69 μmol, 1 eq) in DCM (1 mL) was added HCl/dioxane (2 M, 3 mL, 11.52 eq). The mixture was stirred at 25° C. for 0.25 hr. The mixture was concentrated under reduced pressure to give Intermediate 1-4 (330 mg, crude, HCl salt) as a white solid. LCMS (Method D): Retention time: 0.236 min, [M+H]⁺=534.3.

### Step 3: Synthesis of I-869.

To a solution of Intermediate 1-5 (94.33 mg, 366.59 μmol, 1.1 eq) in DMF (2 mL) was added EDCI (191.66 mg, 999.78 μmol, 3 eq), HOAt (45.36 mg, 333.26 μmol, 46.62 μL, 1 eq), NMM (168.55 mg, 1.67 mmol, 183.20 μL, 5 eq) and Intermediate 1-4 (190 mg, 333.26 μmol, 1 eq, HCl salt). The mixture was stirred at 25° C. for 1 hr. The reaction mixture was poured into water (5 mL) and extracted with EA (3 mL*3). The combined organic layers were dried over Na₂SO₄, filtered and concentrated under reduced pressure to give a residue. The residue was purified by flash silica gel chromatography (ISCO; 12 g SepaFlash Silica Flash Column, Eluent of 0˜100% DCM/(MeOH/DCM=1/4)@25 mL/min, DCM/MeOH=10:1, Rf=0.5) and the eluent was concentrated under reduced pressure to give a residue. To the residue was added H₂O, and the mixture was lyophilized to give I-869 (220 mg, 284.62 μmol, 85.40% yield, 100% purity) as a white solid. LCMS (Method H): Retention time: 0.691 min, [M+H]⁺=773.4. SFC: Retention time: 0.932 min. ¹H NMR (400 MHz, METHANOL-d4) δ=8.50 (d, J=2.0 Hz, 1H), 8.22 (d, J=2.4 Hz, 1H), 7.93-7.88 (m, 1H), 7.84 (s, 1H), 7.77 (d, J=0.8 Hz, 1H), 7.39-7.34 (m, 1H), 6.60-6.42 (m, 1H), 4.46-4.29 (m, 1H), 3.97-3.86 (m, 1H), 3.85-3.71 (m, 3H), 3.71-3.59 (m, 4H), 3.59-3.43 (m, 3H), 3.41 (d, J=4.4 Hz, 4H), 2.81-2.53 (m, 6H), 1.95-1.85 (m, 3H), 1.82-1.49 (m, 9H), 1.45-1.34 (m, 9H), 1.29 (t, J=7.6 Hz, 3H), 1.24-1.08 (m, 3H), 1.07-0.91 (m, 2H).

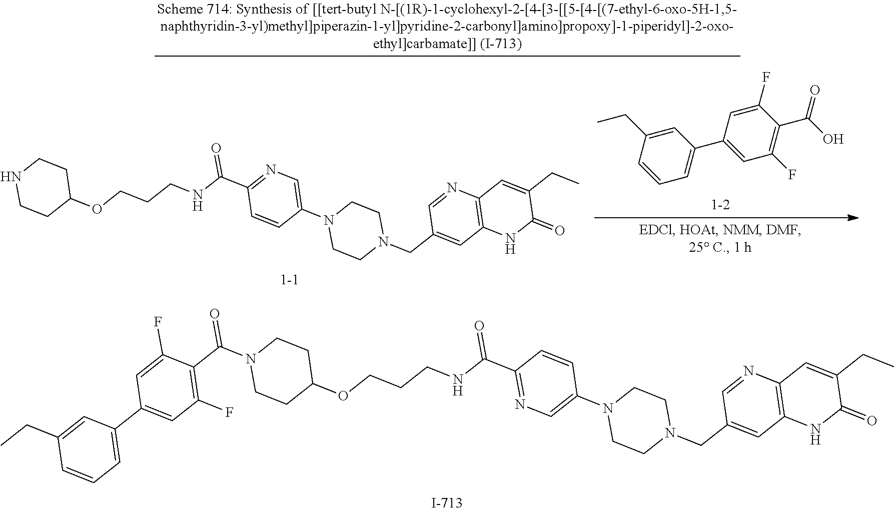

To a solution of Intermediate 1-2 (25.30 mg, 96.47 μmol, 1.1 eq) in DMF (0.5 mL) was added EDCI (50.44 mg, 263.10 μmol, 3 eq), HOAt (11.94 mg, 87.70 μmol, 12.27 μL, 1 eq), NMM (44.35 mg, 438.50 μmol, 48.21 μL, 5 eq) and Intermediate 1-1 (50 mg, 87.70 μmol, 1 eq, HCl salt). The mixture was stirred at 25° C. for 1 hr. The reaction mixture was poured into water (5 mL) and extracted with EA (3 mL*3). The combined organic layers were dried over Na₂SO₄, filtered and concentrated under reduced pressure to give a residue. The residue was purified by flash silica gel chromatography (ISCO; 4 g SepaFlash Silica Flash Column, Eluent of 0˜100% DCM/(MeOH/DCM=1/4) @100 mL/min, DCM/MeOH=10:1, Rf=0.5) and the eluent was concentrated under reduced pressure to give a residue, to the residue was added H₂O, and the mixture was lyophilized to give I-713 (20.16 mg, 25.92 μmol, 29.55% yield) as a white solid. LCMS (Method D): Retention time: 0.426 min, [M+H]⁺=778.2. ¹H NMR (400 MHz, METHANOL-d4) δ=8.46 (d, J=2.0 Hz, 1H), 8.20 (d, J=2.8 Hz, 1H), 7.91 (d, J=8.8 Hz, 1H), 7.82 (s, 1H), 7.74 (d, J=1.2 Hz, 1H), 7.48 (s, 1H), 7.45 (d, J=8.0 Hz, 1H), 7.41-7.32 (m, 4H), 7.25 (d, J=8.0 Hz, 1H), 4.03-3.93 (m, 1H), 3.90-3.79 (m, 1H), 3.72-3.61 (m, 6H), 3.58-3.47 (m, 2H), 3.40-3.34 (m, 4H), 3.30-3.24 (m, 1H), 2.75-2.68 (m, 2H), 2.68-2.60 (m, 6H), 1.99-1.88 (m, 3H), 1.87-1.77 (m, 2H), 1.77-1.68 (m, 1H), 1.30-1.24 (m, 6H). ¹⁹F NMR (376 MHz, METHANOL-d4) δ=−115.03 (1F), −115.3 (1F).

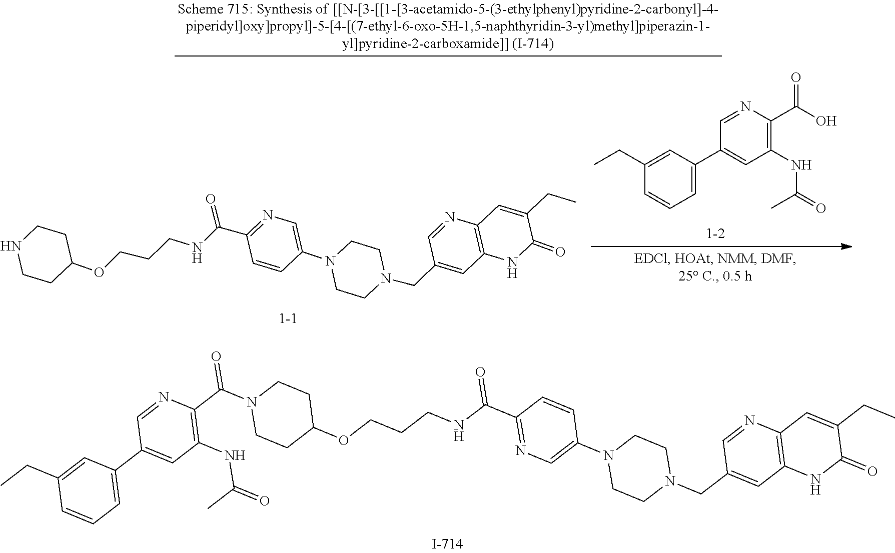

To a solution of Intermediate 1-2 (21.94 mg, 77.18 μmol, 1.1 eq) in DMF (0.5 mL) was added EDCI (40.35 mg, 210.48 μmol, 3 eq), HOAt (9.55 mg, 70.16 μmol, 9.81 μL, 1 eq), NMM (35.48 mg, 350.80 μmol, 38.57 μL, 5 eq) and Intermediate 1-2 (40 mg, 70.16 μmol, 1 eq, HCl salt). The mixture was stirred at 25° C. for 0.5 hr. The reaction mixture was poured into water (3 mL) and extracted with EA (2 mL*3). The combined organic layers were dried over Na₂SO₄, filtered and concentrated under reduced pressure to give a residue. The residue was purified by flash silica gel chromatography (ISCO; 4 g SepaFlash Silica Flash Column, Eluent of 0˜100% DCM/(MeOH/DCM=1/4)@18 mL/min, DCM/MeOH=10:1, Rf=0.5) and the eluent was concentrated under reduced pressure to give a residue, to the residue was added H₂O, and the mixture was lyophilized to give I-714 (16 mg, 19.76 μmol, 28.16% yield, 98.7% purity) as a white solid. LCMS (Method D): Retention time: 0.377 min, [M+H]⁺=800.5. ¹H NMR (400 MHz, METHANOL-d4) δ=8.63 (d, J=2.0 Hz, 1H), 8.46 (d, J=1.6 Hz, 1H), 8.30 (d, J=1.6 Hz, 2H), 7.93 (d, J=8.8 Hz, 1H), 7.84 (s, 1H), 7.73 (s, 1H), 7.50 (s, 1H), 7.48-7.43 (m, 1H), 7.42-7.34 (m, 2H), 7.29 (d, J=7.6 Hz, 1H), 4.04-3.92 (m, 1H), 3.81-3.68 (m, 5H), 3.66 (s, 2H), 3.56 (t, J=6.0 Hz, 2H), 3.40-3.35 (m, 5H), 2.76-2.66 (m, 4H), 2.65-2.53 (m, 4H), 2.18 (s, 3H), 2.06-1.91 (m, 4H), 1.90-1.79 (m, 2H), 1.33-1.26 (m, 6H).

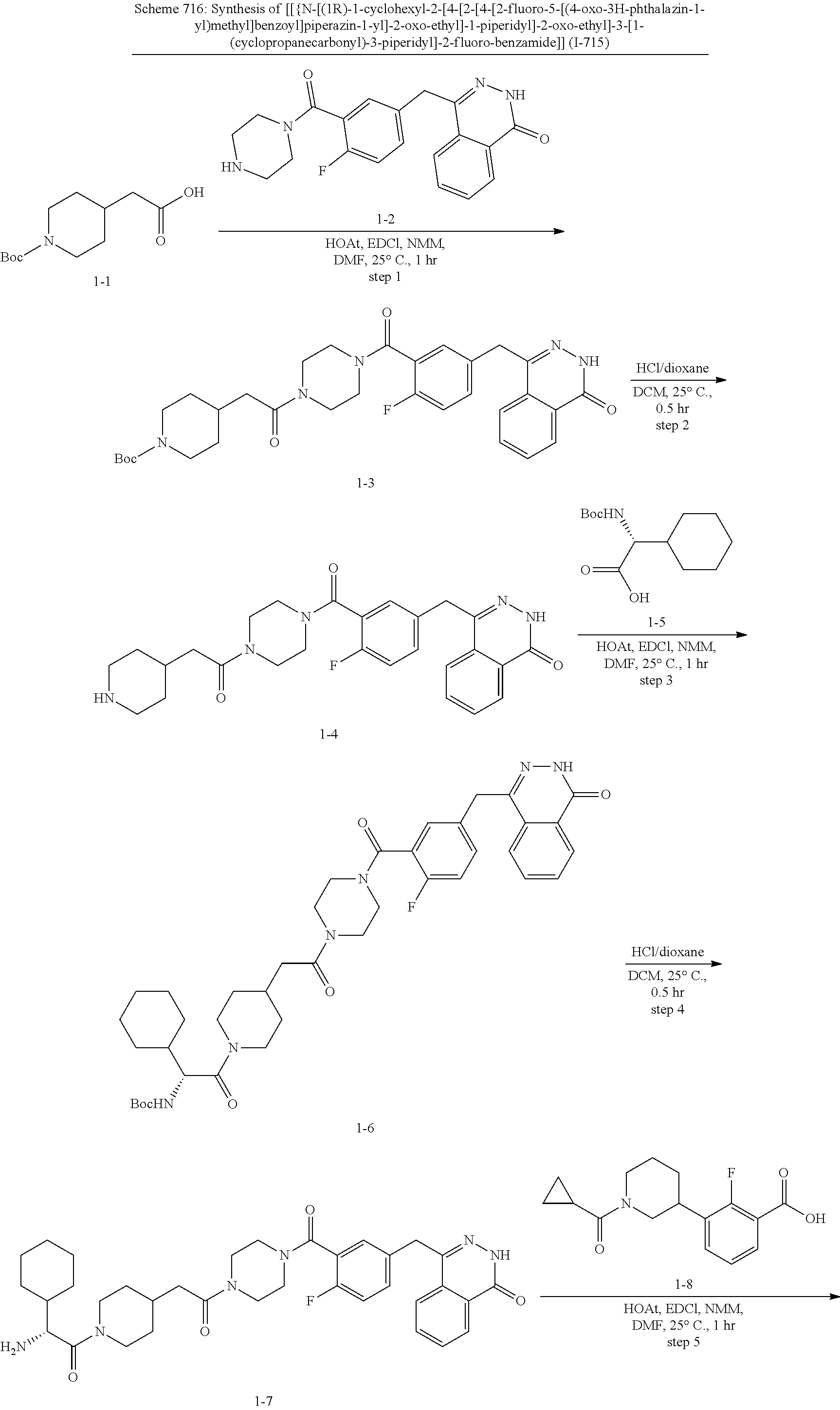

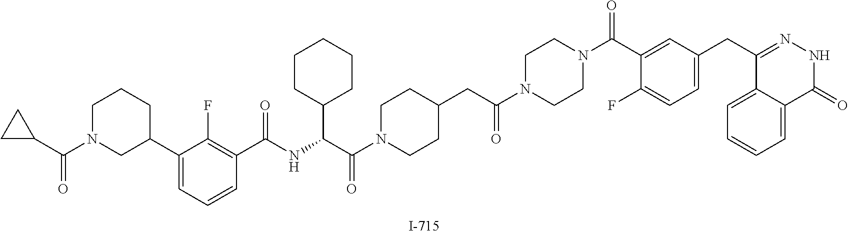 [SMILES: O=C(N[C@@H](C(=O)N1CCC(CC(=O)N2CCN(C(=O)c3cc(Cc4n[nH]c(=O)c5ccccc45)ccc3F)CC2)CC1)C1CCCCC1)c1cccc(C2CCCN(C(=O)C3CC3)C2)c1F]

### Step 1: Synthesis of Intermediate 1-3.

To a solution of Intermediate 1-2 (500 mg, 1.36 mmol, 1 eq) and Intermediate 1-1 (332.02 mg, 1.36 mmol, 1 eq) in DMF (5 mL) was added EDCI (784.83 mg, 4.09 mmol, 3 eq), HOAt (185.75 mg, 1.36 mmol, 190.90 μL, 1 eq) and NMM (690.16 mg, 6.82 mmol, 750.18 μL, 5 eq). The mixture was stirred at 25° C. for 1 hr. The mixture was washed with water (5 mL) and extracted with EA (5 mL*3). The combined organic layers were dried over sodium sulfate, filtered and concentrated under reduced pressure to give a residue. The residue was purified by flash silica gel chromatography (ISCO; 20 g SepaFlash Silica Flash Column, Eluent of 0˜30% MeOH/EA @60 mL/min) and then the eluent was concentrated in vacuo to give Intermediate 1-3 (800 mg, 1.33 mmol, 97.10% yield, 98% purity) as a yellow oil. LCMS (Method D): Rt=0.394 min, [M+H]⁺=592.3.

### Step 2: Synthesis of Intermediate 1-4.

To a solution of Intermediate 1-3 (500 mg, 845.06 μmol, 1 eq) in DCM (5 mL) was added HCl/dioxane (2 M, 5 mL). The mixture was stirred at 25° C. for 0.5 hr. The reaction mixture was concentrated in vacuo. The crude product was used in the next step without further purification. Intermediate 1-4 (500 mg, crude, HCl salt) was obtained as a white solid. LCMS (Method D): Rt=0.249 min, [M+H]⁺=492.4. ¹H NMR (400 MHz, METHANOL-d4) δ=8.36 (d, J=7.6 Hz, 1H), 7.99-7.93 (m, 1H), 7.91-7.81 (m, 2H), 7.49 (s, 1H), 7.38-7.37 (m, 1H), 7.16-7.14 (m, 1H), 4.39 (s, 2H), 3.79 (d, J=5.2 Hz, 1H), 3.76-3.71 (m, 1H), 3.70-3.66 (m, 2H), 3.65-3.64 (m, 1H), 3.54-3.47 (m, 2H), 3.42-3.35 (m, 3H), 3.04-2.97 (m, 2H), 2.48-2.38 (m, 2H), 2.15-2.04 (m, 1H), 1.98 (d, J=12.4 Hz, 2H), 1.54-1.43 (m, 2H). ¹⁹F NMR (376 MHz, METHANOL-d4) δ=−120.694.

### Step 3: Synthesis of Intermediate 1-6.

To a solution of Intermediate 1-5 (243.67 mg, 946.94 μmol, 1 eq) in DMF (5 mL) was added EDCI (544.59 mg, 2.84 mmol, 3 eq), HOAt (128.89 mg, 946.94 μmol, 132.47 μL, 1 eq) and NMM (478.90 mg, 4.73 mmol, 520.54 μL, 5 eq). Then Intermediate 1-4 (500 mg, 946.94 μmol, 1 eq, HCl salt) was added. The mixture was stirred at 25° C. for 1 hr. The mixture was washed with water (5 mL) and extracted with EA (5 mL*3). The combined organic layers were dried over sodium sulfate, filtered and concentrated under reduced pressure to give a residue. The residue was purified by flash silica gel chromatography (ISCO; 20 g SepaFlash Silica Flash Column, Eluent of 0˜20% MeOH/DCM @60 mL/min) and then the eluent was concentrated in vacuo to give Intermediate 1-6 (660 mg, 875.95 μmol, 92.50% yield, 97% purity) as a yellow oil. LCMS (Method D): Rt=0.451 min, [M+H]⁺=731.5. ¹H NMR (400 MHz, CHLOROFORM-d) δ=10.26-10.08 (m, 1H), 8.50-8.43 (m, 1H), 7.81-7.75 (m, 2H), 7.72 (s, 1H), 7.33 (d, J=5.2 Hz, 2H), 7.10-7.03 (m, 1H), 5.41-5.31 (m, 1H), 4.62 (d, J=10.4 Hz, 1H), 4.51-4.42 (m, 1H), 4.29 (s, 2H), 4.01-3.94 (m, 1H), 3.84-3.74 (m, 2H), 3.57 (d, J=8.0 Hz, 2H), 3.43 (s, 1H), 3.30 (s, 2H), 3.15-3.06 (m, 1H), 2.63-2.60 (m, 1H), 2.34-2.13 (m, 3H), 1.76 (d, J=0.8 Hz, 5H), 1.64 (d, J=9.2 Hz, 3H), 1.45-1.42 (m, 9H), 1.25-1.12 (m, 5H), 1.11-0.98 (m, 3H). ¹⁹F NMR (376 MHz, CHLOROFORM-d) δ=−117.552.

### Step 4: Synthesis of Intermediate 1-7.

To a solution of Intermediate 1-6 (550 mg, 752.53 μmol, 1 eq) in DCM (5 mL) was added HCl/dioxane (2 M, 5 mL). The mixture was stirred at 25° C. for 0.5 hr. The reaction mixture was concentrated in vacuo. The crude product was used in the next step without further purification. Intermediate 1-7 (550 mg, crude, HCl salt) was obtained as a white solid. LCMS (Method D): Rt=0.314 min, [M+H]⁺=631.3. ¹H NMR (400 MHz, METHANOL-d4) δ=8.37-8.35 (m, 1H), 7.99-7.94 (m, 1H), 7.91-7.81 (m, 2H), 7.50 (d, J=2.4 Hz, 1H), 7.37 (d, J=5.2 Hz, 1H), 7.17-7.16 (m, 1H), 4.51-4.47 (m, 1H), 4.39 (s, 2H), 4.34-4.26 (m, 1H), 3.95 (d, J=12.4 Hz, 1H), 3.81-3.72 (m, 2H), 3.71-3.67 (m, 2H), 3.55-3.48 (m, 2H), 3.34-3.33 (m, 1H), 3.22-3.13 (m, 1H), 2.81-2.72 (m, 1H), 2.47-2.33 (m, 2H), 2.13-2.03 (m, 1H), 1.82 (d, J=10.0 Hz, 5H), 1.76-1.67 (m, 3H), 1.35-1.26 (m, 3H), 1.25-1.12 (m, 5H). ¹⁹F NMR (376 MHz, METHANOL-d4) δ=−120.711.

### Step 5: Synthesis of I-715.

To a solution of Intermediate 1-8 (240.14 mg, 824.33 μmol, 1 eq) in DMF (5.5 mL) was added EDCI (474.07 mg, 2.47 mmol, 3 eq), HOAt (112.20 mg, 824.33 μmol, 115.31 μL, 1 eq) and NMM (416.89 mg, 4.12 mmol, 453.14 μL, 5 eq). Then Intermediate 1-7 (550 mg, 824.33 μmol, 1 eq, HCl salt) was added. The mixture was stirred at 25° C. for 1 hr. The mixture was washed with water (6 mL) and extracted with EA (8 mL*3). The combined organic layers were dried over sodium sulfate, filtered and concentrated under reduced pressure to give a residue. The residue was purified by reversed phase chromatography (0.1% FA condition), and the eluent was concentrated under reduced pressure to remove ACN and lyophilized to give I-715 (329.23 mg, 354.16 μmol, 42.96% yield, 97.25% purity) as a white solid. LCMS (Method D): Rt=0.473 min, [M+H]⁺=904.4. LCMS (Method): Rt=17.485 min, 17.994 min, [M+H]⁺=904.3, 904.3. SFC: Rt=2.351 min, 3.097 min. ¹H NMR (400 MHz, METHANOL-d4) δ=8.39-8.33 (m, 1H), 7.98-7.90 (m, 1H), 7.90-7.78 (m, 2H), 7.60-7.45 (m, 3H), 7.37 (s, 1H), 7.30-7.21 (m, 1H), 7.16-7.15 (m, 1H), 4.96 (s, 1H), 4.55 (d, J=13.2 Hz, 2H), 4.45-4.34 (m, 3H), 4.26 (d, J=10.4 Hz, 1H), 3.80-3.62 (m, 4H), 3.50-3.48 (m, 2H), 3.39-3.32 (m, 1H), 3.28-3.14 (m, 3H), 3.13-2.92 (m, 1H), 2.86-2.68 (m, 2H), 2.47-2.29 (m, 2H), 2.15-1.98 (m, 3H), 1.92 (d, J=12.4 Hz, 2H), 1.87-1.75 (m, 6H), 1.68 (d, J=10.8 Hz, 3H), 1.35-1.20 (m, 4H), 1.16 (d, J=14.0 Hz, 3H), 0.91-0.77 (m, 4H). ¹⁹F NMR (376 MHz, METHANOL-d4) δ=−120.691 (1F), −121.689 (1F).

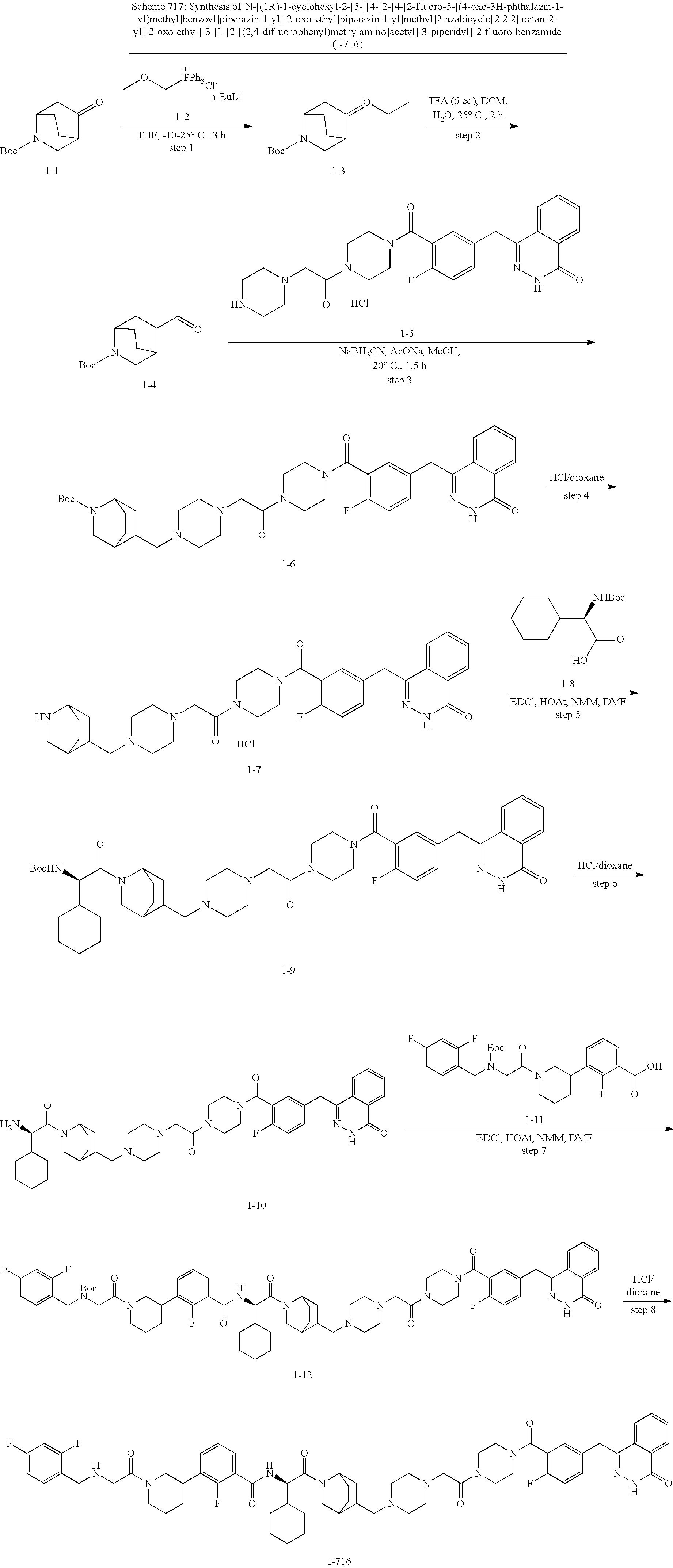

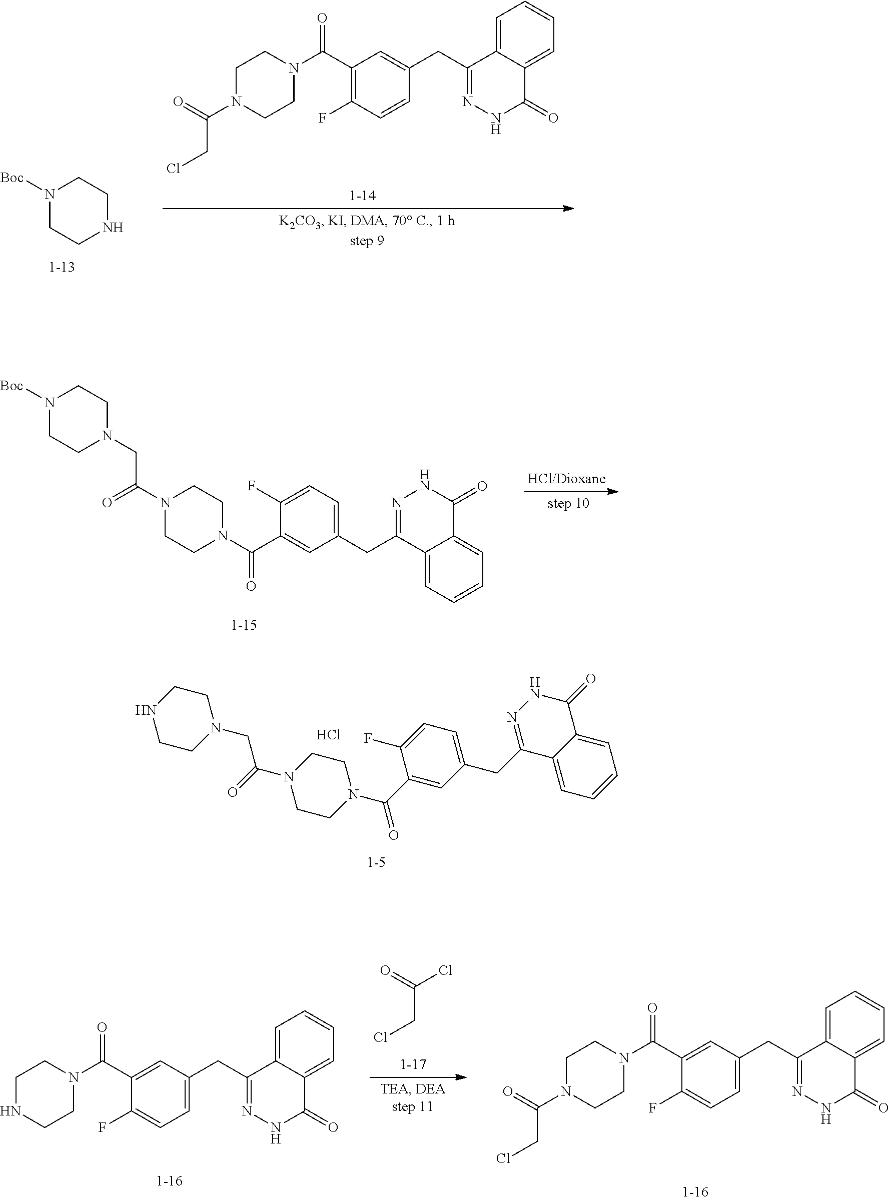

### Step 1: Synthesis of Intermediate 1-3

To a solution of Intermediate 1-2 (11.41 g, 33.29 mmol, 1.5 eq) in THF (25 mL) was added n-BuLi (2.5 M, 13.32 mL, 1.5 eq) dropwise at −10° C. under N₂, and the mixture was stirred for 1 h at 0° C. A solution of Intermediate 1-1 (5 g, 22.19 mmol, 1 eq) in THF (25 mL) was added dropwise to the reaction mixture at −20° C., and the mixture was then stirred for 2 h at 25° C. under N₂ atmosphere. The mixture quenched with sat. NH₄Cl (40 mL) under N₂ atmosphere and extracted with EA (20 mL*3), dried over Na₂SO₄, filtered and concentrated under reduced pressure to give the residue. The crude was purified by Flash Silica-gel chromatography (ISCO; 120 g SepaFlash Silica Flash Column, Eluent of 16-20% EA/PE) and the eluent was combined and concentrated to give Intermediate 1-3 (3.2 g, 12.63 mmol, 56.91% yield) as a colorless oil. ¹H NMR (400 MHz, CHLOROFORM-d) δ=5.99-5.67 (m, 1H), 4.28-3.98 (m, 1H), 3.62-3.49 (m, 3H), 3.43-3.25 (m, 2H), 3.03-2.92 (m, 1H), 2.62-2.41 (m, 1H), 2.35-2.17 (m, 1H), 1.99-1.84 (m, 1H), 1.78-1.60 (m, 3H), 1.46 (s, 9H).

### Step 2: Synthesis of Intermediate 1-4

To a solution of Intermediate 1-3 (3 g, 11.84 mmol, 1 eq) in DCM (24 mL) was added H₂O (8 mL) and TFA (8.10 g, 71.05 mmol, 5.28 mL, 6 eq). The resulting mixture was stirred at 25° C. for 2 hrs. The reaction mixture was quenched by saturated NaHCO₃ (40 mL), and extracted by DCM (15 mL*3). The organic phase was combined and concentrated to afford the crude. The crude product was purified by flash silica gel chromatography (ISCO; 80 g SepaFlash Silica Flash Column, Eluent of 0˜20% EA/PE@60 mL/min). After purification, the eluent was concentrated to give Intermediate 1-4 (2.4 g, 10.03 mmol, 84.69% yield) as a colorless oil. LCMS (Method E): Rt=0.482 min, [M+H-tBu]⁺=184.1. ¹H NMR (400 MHz, CHLOROFORM-d) δ=9.73 (d, J=9.8 Hz, 1H), 4.11-3.96 (m, 1H), 3.52-3.17 (m, 2H), 2.73-2.51 (m, 1H), 2.47-2.08 (m, 2H), 1.99-1.85 (m, 1H), 1.83-1.71 (m, 2H), 1.68-1.50 (m, 2H), 1.47-1.42 (m, 9H).

### Step 3: Synthesis of Intermediate 1-6

To a solution of Intermediate 1-5 (4.20 g, 7.94 mmol, 1 eq, HCl salt) in MeOH (20 mL) was added NaOAc (2.61 g, 31.76 mmol, 4 eq), and the mixture was stirred at 20° C. for 15 min, then Intermediate 1-4 (1.9 g, 7.94 mmol, 1 eq) was added and the resulting suspension was stirred at 20° C. for another 15 min. NaBH₃CN (997.87 mg, 15.88 mmol, 2 eq) was added. The resulting suspension was stirred at 20° C. for another 1 h under N₂. The mixture was quenched with water (50 mL) and extracted with EA (20 mL*3), dried over Na₂SO₄, filtered and concentrated under reduced pressure to give a residue. The crude product was purified by flash silica gel chromatography (ISCO; 120 g SepaFlash Silica Flash Column, Eluent of 0˜20% MeOH/DCM@60 mL/min). Then the eluent was concentrated to give Intermediate 1-6 (3.9 g, 5.45 mmol, 68.62% yield) as a white solid. LCMS (Method E): Rt=0.437 min, [M+H]⁺=716.3. SFC: Rt=2.007 min, 2.158 min, 2.628 min. ¹H NMR (400 MHz, DMSO-d₆) δ=12.59 (s, 1H), 8.26 (d, J=7.6 Hz, 1H), 8.00-7.80 (m, 3H), 7.50-7.32 (m, 2H), 7.24 (t, J=8.8 Hz, 1H), 4.33 (s, 2H), 4.09 (d, J=1.2 Hz, 1H), 3.90-3.76 (m, 1H), 3.69-3.49 (m, 4H), 3.47-3.04 (m, 16H), 2.08-1.96 (m, 1H), 1.85-1.50 (m, 6H), 1.38 (d, J=5.6 Hz, 10H), 1.28-1.07 (m, 1H).

### Step 4: Synthesis of Intermediate 1-7

To a solution of Intermediate 1-6 (3.9 g, 5.45 mmol, 1 eq) in DCM (20 mL) was added HCl/dioxane (2M, 20 mL), and the reaction mixture was stirred at 25° C. for 1 h. The reaction mixture was concentrated to give crude product. The crude product was used in the next step directly. Intermediate 1-7 (4.5 g, crude, HCl salt) was obtained as a white solid. LCMS (Method E): Rt=0.374 min, [M+H]=616.4. SFC: Rt=1.233 min, 1.289 min, 1.507 min, 1.593 min. ¹H NMR (400 MHz, DMSO-d₆) δ=12.61 (d, J=4.8 Hz, 1H), 9.48-8.87 (m, 2H), 8.26 (d, J=7.6 Hz, 1H), 8.02-7.80 (m, 3H), 7.51-7.33 (m, 2H), 7.25 (t, J=9.2 Hz, 1H), 3.82-3.59 (m, 13H), 3.47-3.05 (m, 12H), 2.43-2.07 (m, 3H), 2.03-1.90 (m, 2H), 1.76-1.29 (m, 4H). ¹⁹F NMR (377 MHz, DMSO-d₆) δ=−119.718.

### Step 5: Synthesis of Intermediate 1-9

To a solution of Intermediate 1-8 (1.38 g, 5.37 mmol, 1 eq) in DMF (35 mL) was added HOAt (1.10 g, 8.05 mmol, 1.13 mL, 1.5 eq), EDCI (3.09 g, 16.10 mmol, 3 eq), NMM (2.71 g, 26.83 mmol, 2.95 mL, 5 eq) and Intermediate 1-7 (3.5 g, 5.37 mmol, 1 eq, HCl salt). The resulting mixture was stirred at 25° C. for 1 hr. The reaction mixture was quenched by water (40 mL), extracted by EA (20 mL*3), and washed by brine (50 mL*2). The organic phase was combined and concentrated to afford the crude. The crude product was purified by reverse phase chromatography (120 g of XB—C18, 20-40 μm, 120 Å) Mobile phase: A for H₂O (0.1% FA v/v) and B for acetonitrile; Gradient: B 5%-100% in 30 min; Flow rate: 100 ml/min; Column temperature: R.T. Wavelength: 220 nm/254 nm), then the eluent was concentrated to remove organic solvents and lyophilized to give Intermediate 1-9 (3.1 g, 3.63 mmol, 67.56% yield) as a yellow solid. LCMS (Method E): Rt=0.470 min, [M+H]⁺=855.4. SFC: Rt=2.384 min, 2.588 min, 3.072 min, 3.277 min. ¹H NMR (400 MHz, DMSO-d₆) δ=12.60 (s, 1H), 8.26 (d, J=7.6 Hz, 1H), 8.02-7.78 (m, 3H), 7.49-7.32 (m, 2H), 7.23 (t, J=8.8 Hz, 1H), 6.93-6.70 (m, 1H), 4.33 (s, 2H), 3.66-3.41 (m, 7H), 3.28-3.04 (m, 6H), 2.44-2.14 (m, 9H), 1.96-1.79 (m, 3H), 1.75-1.41 (m, 11H), 1.35 (s, 9H), 1.14-0.82 (m, 6H). ¹⁹F NMR (377 MHz, DMSO-d₆) δ=−119.789.

### Step 6: Synthesis of Intermediate 1-10

To a solution of Intermediate 1-9 (4.5 g, 5.26 mmol, 1 eq) in DCM (10 mL) was added HCl/dioxane (2M, 30 mL), and the reaction mixture was stirred at 25° C. for 1 h. The reaction mixture was concentrated to give crude product. The crude product was used in the next step without any other purification. Intermediate 1-10 (4.5 g, crude, HCl salt) was obtained as a white solid. LCMS (Method E): Rt=0.393 min, [M+H]⁺=755.5. ¹H NMR (400 MHz, DMSO-d6) δ=12.62 (d, J=6.2 Hz, 1H), 8.55-8.19 (m, 4H), 8.02-7.78 (m, 3H), 7.50-7.33 (m, 2H), 7.25 (t, J=9.2 Hz, 1H), 4.54-4.14 (m, 6H), 4.12-3.95 (m, 2H), 3.85-3.64 (m, 8H), 3.52-3.17 (m, 9H), 2.36-2.11 (m, 2H), 2.04-1.47 (m, 11H), 1.23-0.93 (m, 5H). ¹⁹F NMR (377 MHz, DMSO-d6) δ=−119.730.

### Step 7: Synthesis of Intermediate 1-12

To a solution of Intermediate 1-11 (352.01 mg, 694.98 μmol, 1 eq) in DMF (6 mL) was added EDCI (399.68 mg, 2.08 mmol, 3 eq), HOAt (141.89 mg, 1.04 mmol, 145.83 μL, 1.5 eq), NMM (351.48 mg, 3.47 mmol, 382.05 μL, 5 eq) and Intermediate 1-10 (550 mg, 694.98 μmol, 1 eq, HCl salt), then the reaction mixture was stirred at 25° C. for 1 h. The reaction mixture was quenched with water (40 mL), extracted by EA (20 mL*3), and washed by brine (50 mL*2). The organic phase was combined and concentrated to afford the crude. The crude product was purified by reverse phase chromatography (120 g of XB—C18, 20-40 μm, 120 Å) Mobile phase: A for H₂O (0.1% FA v/v) and B for acetonitrile; Gradient: B5%-100% in 30 min; Flow rate: 100 ml/min; Column temperature: R.T. Wavelength: 220 nm/254 nm), then the eluent was concentrated to remove organic solvents and adjust the pH to 7 with a saturated solution of NaHCO₃ and the mixture was then extracted by EA (20 mL*3). The organic phase was combined and concentrated to afford Intermediate 1-12 (500 mg, 402.11 μmol, 57.86% yield) as a white solid. LCMS (Method E): Rt=0.543 min, [M+H]⁺=1243.6. SFC: Rt=1.817 min, 2.037 min, 2.313 min. ¹H NMR (400 MHz, DMSO-d₆) δ=12.59 (d, J=2.0 Hz, 1H), 8.48-8.21 (m, 2H), 7.99-7.93 (m, 1H), 7.92-7.79 (m, 2H), 7.51-7.34 (m, 5H), 7.28-7.15 (m, 3H), 7.04 (br d, J=9.0 Hz, 1H), 4.67-4.30 (m, 6H), 4.11 (br d, J=9.2 Hz, 2H), 3.97-3.71 (m, 2H), 3.69-3.41 (m, 6H), 3.21-2.85 (m, 7H), 2.78-2.55 (m, 2H), 2.46-2.17 (m, 9H), 1.95-1.50 (m, 17H), 1.49-1.22 (m, 12H), 1.13-0.94 (m, 4H). ¹⁹F NMR (377 MHz, DMSO-d₆) δ=−112.01 (br d, J=4.5 Hz, 1F), −113.56-−114.46 (m, 1F), −119.50-−120.81 (m, 2F).

### Step 8: Synthesis of I-716

To a solution of Intermediate 1-12 (450 mg, 361.90 μmol, 1 eq) in DCM (2.5 mL) was added HCl/dioxane (2M, 2.5 mL), and the result reaction mixture was stirred at 25° C. for 1 h. The reaction mixture was concentrated to give crude product. The crude product was purified by reversed phase chromatography twice (100 g of XB—C18, 20-40 μm, 120 Å) Mobile phase: A for H₂O (0.1% NH₃·H₂O v/v) and B for acetonitrile; Gradient: B 40%-50% in 10 min; Flow rate: 100 ml/min; Column temperature: R.T. Wavelength: 220 nm/254 nm). Then the eluent was concentrated to remove organic solvents and lyophilized to give I-716 (260 mg, 227.41 μmol, 62.84% yield, 100% purity) as a white solid. LCMS (Method E): Rt=0.465 min, [M+H]⁺=1143.7. SFC: Rt=2.417 min, 2.728 min, 3.220 min. ¹H NMR (400 MHz, DMSO-d₆) δ=12.60 (d, J=1.6 Hz, 1H), 8.50-8.32 (m, 1H), 8.27 (d, J=7.6 Hz, 1H), 8.01-7.94 (m, 1H), 7.93-7.79 (m, 2H), 7.55-7.34 (m, 5H), 7.28-6.99 (m, 4H), 4.72-4.40 (m, 2H), 4.36-3.99 (m, 3H), 3.88-3.36 (m, 12H), 3.28-2.84 (m, 7H), 2.75-2.53 (m, 2H), 2.46-2.19 (m, 9H), 2.08 (s, 1H), 1.87 (br s, 4H), 1.80-1.30 (m, 13H), 1.28-0.94 (m, 6H). ¹⁹F NMR (377 MHz, DMSO-d₆) δ=−112.50-−112.79 (m, 1F), −114.99 (d, J=6.8 Hz, 1F), −119.78 (s, 1F), −119.86-−120.53 (m, 1F).

### Step 9: Synthesis of Intermediate 1-15

To a solution of Intermediate 1-14 (3 g, 6.77 mmol, 1 eq) and Intermediate 1-13 (1.32 g, 7.09 mmol, 1.05 eq) in DMA (30 mL) was added KI (112.45 mg, 677.40 μmol, 0.1 eq) and K₂CO₃ (3.74 g, 27.10 mmol, 4 eq). The mixture was stirred at 70° C. for 1 hr. The reaction mixture was quenched by a saturated solution of NaHCO₃ (50 mL) then extracted by DCM (50 ml*3), the combined organic phase was washed by brine (200 mL), dried by anhydrous Na₂SO₄, filtered and concentrated to give product. The crude product was purified by flash silica gel chromatography (ISCO; 80 g SepaFlash Silica Flash Column, Eluent of 0˜20% MeOH/DCM@60 mL/min). The eluent was concentrated to give Intermediate 1-15 (4 g, 6.75 mmol, 80.00% yield) as a yellow oil. LCMS (Method E): Rt=0.413 min, [M+H]⁺=593.3. ¹H NMR (400 MHz, CHLOROFORM-d) δ=10.57 (d, J=16.0 Hz, 1H), 8.58-8.34 (m, 1H), 7.80-7.66 (m, 3H), 7.38-7.30 (m, 2H), 7.05 (t, J=8.0 Hz, 1H), 3.92-3.66 (m, 4H), 3.63-3.54 (m, 2H), 3.50-3.38 (m, 6H), 3.37-3.17 (m, 4H), 2.45 (d, J=15.2 Hz, 4H), 1.46 (s, 9H), 1.27 (d, J=7.2 Hz, 1H). ¹⁹F NMR (377 MHz, CHLOROFORM-d) δ=−117.668.

### Step 10: Synthesis of Intermediate 1-5

A solution of Intermediate 1-15 (4 g, 6.75 mmol, 1 eq) in HCl/dioxane (2M, 40 mL) was stirred for 1 h at 25° C. The reaction mixture was concentrated to give crude product. The crude product was used in the next step directly without any other purification. Intermediate 1-5 (4.5 g, crude, HCl salt) was obtained as a yellow solid. LCMS (Method E): Rt=0.369 min, [M+H]⁺=493.2. ¹H NMR (400 MHz, DMSO-d₆) δ=12.61 (d, J=4.0 Hz, 1H), 10.42-9.70 (m, 2H), 8.26 (d, J=7.6 Hz, 1H), 8.04-7.79 (m, 3H), 7.46 (d, J=6.4 Hz, 1H), 7.40-7.33 (m, 1H), 7.25 (t, J=9.2 Hz, 1H), 4.51 (d, J=19.6 Hz, 6H), 3.75-3.60 (m, 4H), 3.51-3.37 (m, 6H), 3.35-3.18 (m, 3H).

### Step 11: Synthesis of Intermediate 1-14

To a solution of Intermediate 1-16 (40 g, 109.17 mmol, 1 eq) and Intermediate 1-17 (24.66 g, 218.35 mmol, 17.39 mL, 2 eq) in DCM (400 mL) was added TEA (33.14 g, 327.52 mmol, 45.59 mL, 3 eq). The mixture was stirred at 0° C. for 1 hr. The reaction mixture was quenched by addition of H₂O (200 mL), and then diluted with DCM (200 mL) and extracted with DCM (400 mL*3). The combined organic layers were dried over anhydrous Na₂SO₄, filtered and concentrated under reduced pressure to give a residue. The residue was purified by flash silica gel chromatography (ISCO; 330 g SepaFlash Silica Flash Column, Eluent of 0˜100% Ethyl acetate/Petroleum ether gradient @150 mL/min) and the eluent was concentrated under reduced pressure to give Intermediate 1-14 (55 g, crude) as an off-white solid. LCMS (Method E): Rt=0.431 min, [M+H]⁺=443.2. ¹H NMR (400 MHz, DMSO-d₆) δ=12.60 (s, 1H), 8.27-8.25 (m, 1H), 7.98-7.94 (m, 1H), 7.90-7.87 (m, 1H), 7.85-7.80 (m, 1H), 7.48-7.35 (m, 2H), 7.26-7.21 (m, 1H), 4.46-4.36 (m, 2H), 4.33 (s, 2H), 3.72-3.59 (m, 2H), 3.54 (s, 2H), 3.40 (s, 2H), 3.21 (d, J=16.4 Hz, 2H). ¹⁹F NMR (377 MHz, DMSO-d₆) δ=−119.694.

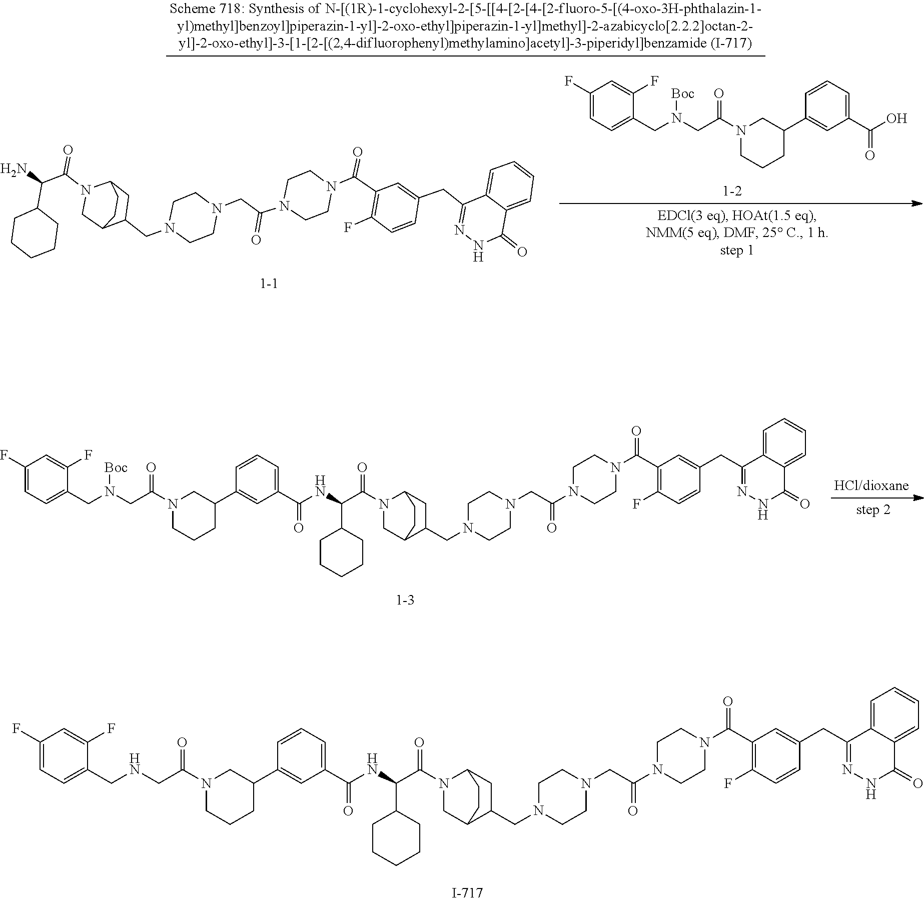 [SMILES: N[C@@H](C(=O)N1CC2CCC1CC2CN1CCN(CC(=O)N2CCN(C(=O)c3cc(Cc4n[nH]c(=O)c5ccccc45)ccc3F)CC2)CC1)C1CCCCC1 ; CC(C)(C)OC(=O)N(CC(=O)N1CCCC(c2cccc(C(=O)O)c2)C1)Cc1ccc(F)cc1F ; CC(C)(C)OC(=O)N(CC(=O)N1CCCC(c2cccc(C(=O)N[C@@H](C(=O)N3CC4CCC3CC4CN3CCN(CC(=O)N4CCN(C(=O)c5cc(Cc6n[nH]c(=O)c7ccccc67)ccc5F)CC4)CC3)C3CCCCC3)c2)C1)Cc1ccc(F)cc1F ; O=C(N[C@@H](C(=O)N1CC2CCC1CC2CN1CCN(CC(=O)N2CCN(C(=O)c3cc(Cc4n[nH]c(=O)c5ccccc45)ccc3F)CC2)CC1)C1CCCCC1)c1cccc(C2CCCN(C(=O)CNCc3ccc(F)cc3F)C2)c1]

### Step 1: Synthesis of Intermediate 1-3

To a solution of Intermediate 1-2 (370.38 mg, 758.16 μmol, 1 eq) in DMF (6 mL) was added EDCI (436.02 mg, 2.27 mmol, 3 eq), HOAt (154.79 mg, 1.14 mmol, 159.08 μL, 1.5 eq), NMM (383.44 mg, 3.79 mmol, 416.78 μL, 5 eq) and Intermediate 1-1 (600 mg, 758.16 μmol, 1 eq, HCl salt), then the reaction mixture was stirred at 25° C. for 1 h. The reaction mixture was quenched with water (40 mL) and extracted by EA (20 mL*3), washed by brine (50 mL*2). The organic phase was combined and concentrated to afford the crude. The crude product was purified by reverse phase chromatography (120 g of XB—C18, 20-40 Mm, 120 Å) Mobile phase: A for H₂O (0.1% FA v/v) and B for acetonitrile; Gradient: B 5%-100% in 30 min; Flow rate: 100 ml/min; Column temperature: R.T. Wavelength: 220 nm/254 nm). After purification, the organic solvents were removed and the remaining aqueous phase was adjusted to pH=7 with a saturated solution of NaHCO₃ and extracted by EA (20 mL*3). The organic phase was combined and concentrated to afford Intermediate 1-3 (450 mg, 367.21 μmol, 48.44% yield) as a white solid. LCMS (Method E): Rt=0.541 min, [M+H]⁺=1225.6. SFC: Rt=1.481 min, 1.572 min, 1.753 min, 1.889 min, 2.259 min. ¹H NMR (400 MHz, DMSO-d₆) δ=12.60 (d, J=2.4 Hz, 1H), 8.58-8.41 (m, 1H), 8.26 (d, J=7.4 Hz, 1H), 8.00-7.93 (m, 1H), 7.91-7.72 (m, 4H), 7.46-7.35 (m, 5H), 7.27-7.17 (m, 2H), 7.14-7.00 (m, 1H), 4.69-4.28 (m, 7H), 4.21-4.08 (m, 2H), 3.84-3.72 (m, 1H), 3.69-3.43 (m, 6H), 3.23-2.96 (m, 6H), 2.71-2.56 (m, 4H), 2.43-2.22 (m, 8H), 1.89 (d, J=12.8 Hz, 5H), 1.81-1.51 (m, 12H), 1.48-1.26 (m, 12H), 1.14-0.88 (m, 4H). ¹⁹F NMR (377 MHz, DMSO-d₆) δ=−111.70-−112.30 (m, 1F), −113.75-−114.30 (m, 1F), −119.78 (br s, 1F).

### Step 2: Synthesis of I-717

To a solution of Intermediate 1-3 (400 mg, 326.41 μmol, 1 eq) in DCM (2 mL) was added HCl/dioxane (2M, 2 mL), then the resulting reaction mixture was stirred at 25° C. for 0.5 h. The reaction mixture was concentrated to give crude product. The crude product was purified by reverse phase chromatography (100 g of XB—C18, 20-40 μm, 120 Å) Mobile phase: A for H₂O (0.1% NH₃·H₂O v/v) and B for acetonitrile; Gradient: B 40%-50% in 10 min; Flow rate: 100 ml/min; Column temperature: R.T. Wavelength: 220 nm/254 nm), then the eluent was concentrated to remove organic solvents and lyophilized to give I-717 (280 mg, 248.82 μmol, 76.23% yield, 100% purity) as a white solid. LCMS (Method E): Rt=0.444 min, [M+H]⁺=1125.4. SFC: Rt=6.595 min, 7.826 min. ¹H NMR (400 MHz, DMSO-d₆) δ=12.60 (s, 1H), 8.58-8.40 (m, 1H), 8.26 (d, J=8.0 Hz, 1H), 8.00-7.93 (m, 1H), 7.93-7.73 (m, 4H), 7.54-7.34 (m, 5H), 7.29-7.12 (m, 2H), 7.10-6.97 (m, 1H), 4.69-4.40 (m, 2H), 4.37-4.08 (m, 3H), 3.94-3.35 (m, 12H), 3.29-2.95 (m, 6H), 2.78-2.59 (m, 2H), 2.46-2.11 (m, 10H), 1.90 (br s, 5H), 1.81-1.41 (m, 12H), 1.27-0.89 (m, 6H). ¹⁹F NMR (377 MHz, DMSO-d₆) δ=−112.7, −115.0, −119.8.

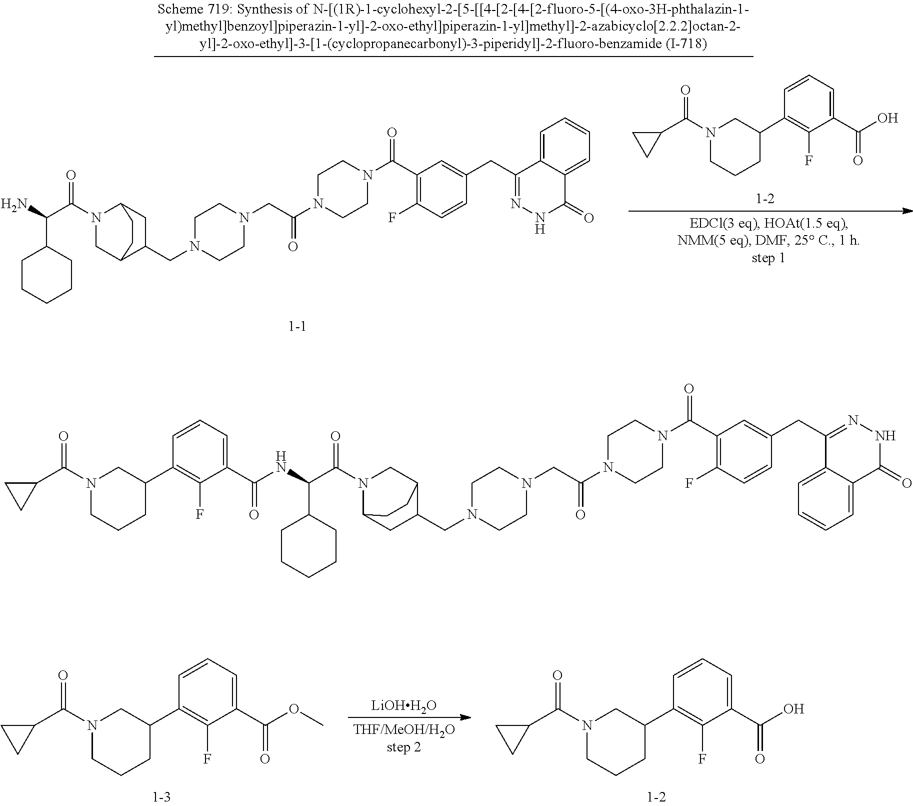

### Step 1: Synthesis of I-718

To a solution of Intermediate 1-2 (184.05 mg, 631.80 μmol, 1 eq) in DMF (5 mL) was added EDCI (363.35 mg, 1.90 mmol, 3 eq), HOAt (128.99 mg, 947.69 μmol, 132.57 μL, 1.5 eq), NMM (319.53 mg, 3.16 mmol, 347.32 μL, 5 eq) and Intermediate 1-1 (500 mg, 631.80 μmol, 1 eq, HCl salt). The reaction mixture was stirred at 25° C. for 1 h. The mixture quenched with water (20 mL) and extracted with EA (10 mL*5), washed by brine (10 mL*2), and the combined organic was dried over Na₂SO₄, filtered and concentrated under reduced pressure to give the residue. The crude product was purified by prep-HPLC (column: Waters Xbridge Prep OBD C18 150*40 mm*10 um; mobile phase: [water (NH₄HCO₃)-ACN]; gradient: 33%-63% B over 15 min), and then the eluent was concentrated to remove organic solvents and lyophilized to give I-718 (300 mg, 291.06 μmol, 46.07% yield, 99.76% purity) as a white solid. LCMS (Method E): Rt=0.481 min, [M+H]⁺=1028.7. SFC: Rt=2.411 min, 12.705 min, 3.167 min, 3.654 min ¹H NMR (400 MHz, DMSO-d₆) δ=12.59 (s, 1H), 8.38 (d, J=4.4 Hz, 1H), 8.26 (d, J=7.6 Hz, 1H), 8.00-7.93 (m, 1H), 7.93-7.79 (m, 2H), 7.54-7.33 (m, 4H), 7.28-7.18 (m, 2H), 4.68-4.39 (m, 2H), 4.37-4.08 (m, 4H), 3.68-3.43 (m, 5H), 3.38-3.34 (m, 1H), 3.26-2.79 (m, 7H), 2.77-2.53 (m, 2H), 2.45-2.25 (m, 7H), 1.89 (br s, 5H), 1.81-1.54 (m, 12H), 1.47-1.22 (m, 3H), 1.20-0.97 (m, 6H), 0.86 (t, J=7.2 Hz, 1H), 0.71 (d, J=12.8 Hz, 4H). ¹⁹F NMR (377 MHz, DMSO-d₆) δ=−119.78, −120.30.

### Step 2: Synthesis of Intermediate 1-2

To a solution of Intermediate 1-3 (1 g, 3.28 mmol, 1 eq) in MeOH (4 mL) and THF (4 mL) was added a solution of LiOHH₂O (412.29 mg, 9.83 mmol, 3 eq) in H₂O (2 mL). The resulting mixture was stirred at 25° C. for 1 h. The reaction mixture was concentrated to give a mixture, and the mixture was diluted with water (5 mL) and then adjusted to pH=6 by 1 N aq·HCl and extracted with EA (5 mL*3). The combined organic phase was dried by anhydrous Na₂SO₄, filtered and concentrated to give product. The crude product was used directly. Intermediate 1-2 (700 mg, 2.40 mmol, 73.37% yield) was obtained as a white solid. LCMS (Method E): Rt=0.454 min, [M+H]⁺=292.2. SFC: Rt=1.341 min, 1.461 min. ¹H NMR (400 MHz, DMSO-d₆) δ=7.77-7.54 (m, 2H), 7.26 (br s, 1H), 4.52-4.18 (m, 2H), 3.22-2.79 (m, 3H), 2.74-2.57 (m, 1H), 1.94-1.69 (m, 3H), 1.64-1.38 (m, 1H), 0.72 (br s, 4H).

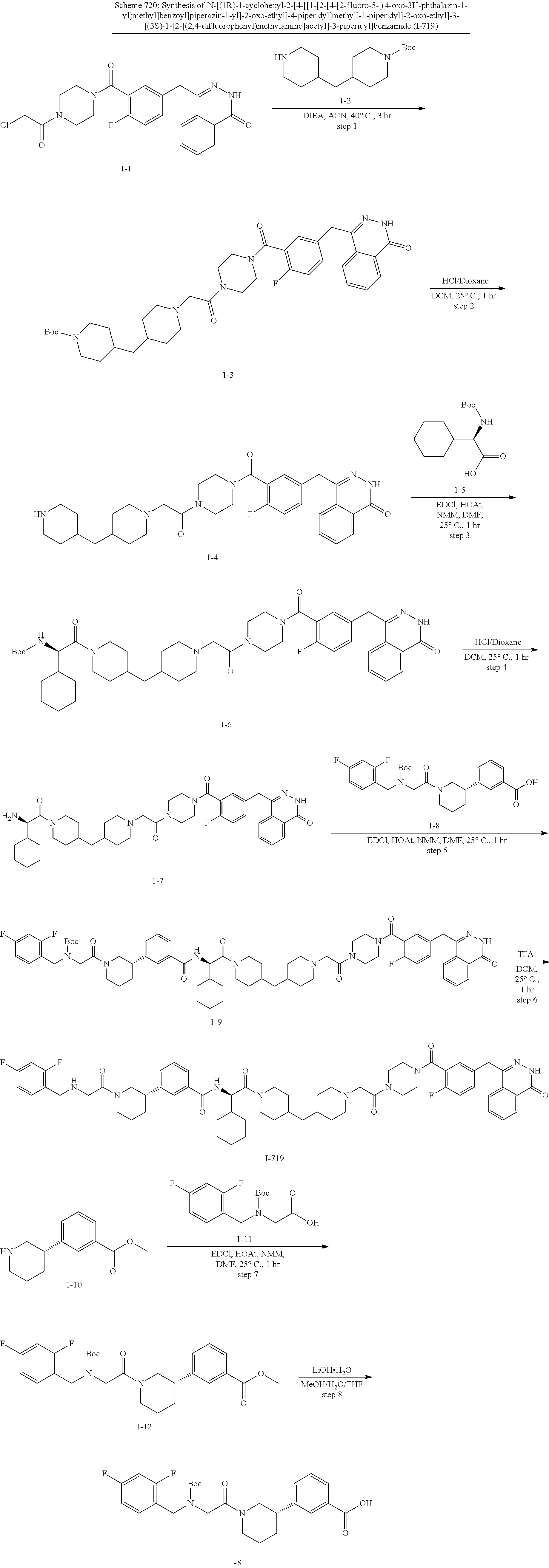

### Step 1: Synthesis of Intermediate 1-3

To a solution of Intermediate 1-1 (2 g, 4.52 mmol, 1 eq) in ACN (20 mL) was added DIEA (1.17 g, 9.03 mmol, 1.57 mL, 2 eq) and Intermediate 1-2 (1.53 g, 5.42 mmol, 1.2 eq). The mixture was stirred at 40° C. for 2 hr. The residue was diluted with H₂O (30 mL) and extracted with EA (30 mL*3), dried over Na₂SO₄, filtered and concentrated under reduced pressure to give a residue. The residue was purified by flash silica gel chromatography (ISCO; 40 g SepaFlash Silica Flash Column, Eluent of 0˜100% Petroleum ether/Ethyl acetate@60 mL/min). The eluent was concentrated under reduced pressure to give Intermediate 1-3 (2.7 g, 3.81 mmol, 84.36% yield, 97.1% purity) as a white solid. LCMS (Method D): Rt=0.379 min, [M+H]⁺=689.3. ¹H NMR (400 MHz, METHANOL-d4) δ=8.37 (d, J=7.6 Hz, 1H), 7.99-7.92 (m, 1H), 7.90-7.79 (m, 2H), 7.48 (s, 1H), 7.38 (t, J=5.6 Hz, 1H), 7.16 (t, J=8.8 Hz, 1H), 4.38 (s, 2H), 4.07-3.99 (m, 2H), 3.82-3.62 (m, 4H), 3.60-3.47 (m, 2H), 3.33 (s, 1H), 3.30-3.26 (m, 1H), 3.25-3.14 (m, 2H), 2.94-2.83 (m, 2H), 2.73 (s, 2H), 2.12-2.02 (m, 2H), 1.65 (d, J=11.2 Hz, 4H), 1.55 (d, J=6.4 Hz, 1H), 1.44 (s, 9H), 1.43 (s, 1H), 1.41-1.30 (m, 1H), 1.18-1.14 (m, 3H), 1.07-0.93 (m, 2H). ¹⁹F NMR (377 MHz, METHANOL-d₄), δ=−120.734.

### Step 2: Synthesis of Intermediate 1-4

To a solution of Intermediate 1-3 (2.5 g, 3.63 mmol, 1 eq) in DCM (12 mL) was added HCl/dioxane (2 M, 25.00 mL, 13.78 eq). The mixture was stirred at 25° C. for 1 hr. The reaction liquid was concentrated under reduced pressure to obtain the crude product. The crude product was used in the next step without further purification. Intermediate 1-4 (2.4 g, crude, HCl salt) was obtained as a white solid. LCMS (Method D): Rt=0.275 min, [M+H]⁺=589.4.

### Step 3: Synthesis of Intermediate 1-6

To a solution of Intermediate 1-5 (800 mg, 3.11 mmol, 1 eq) in DMF (20 mL) was added HOAt (423.16 mg, 3.11 mmol, 434.90 μL, 1 eq), EDCI (1.79 g, 9.33 mmol, 3 eq), NMM (1.57 g, 15.54 mmol, 1.71 mL, 5 eq) and Intermediate 1-4 (2.33 g, 3.73 mmol, 1.2 eq, HCl salt). The mixture was stirred at 25° C. for 1 hr. The residue was diluted with H₂O (15 mL) and extracted with EA (15 mL*3), dried over Na₂SO₄, filtered and concentrated under reduced pressure to give a residue. The residue was purified by flash silica gel chromatography (ISCO; 40 g SepaFlash Silica Flash Column, Eluent of 0˜100% Dichloromethane/Methanol@60 mL/min). The eluent was concentrated under reduced pressure to give Intermediate 1-6 (2 g, 2.32 mmol, 74.69% yield, 96.13% purity) as a white solid. LCMS (Method D): Rt=0.395 min, [M+H]=828.8. SFC: Rt=1.321 min. ¹H NMR (400 MHz, METHANOL-d4) δ=8.37 (d, J=7.6 Hz, 1H), 7.98-7.94 (m, 1H), 7.90-7.80 (m, 2H), 7.40-7.48 (m, 1H), 7.38 (t, J=6.0 Hz, 1H), 7.16 (t, J=8.8 Hz, 1H), 4.55-4.43 (m, 1H), 4.39 (s, 2H), 3.79 (d, J=4.4 Hz, 1H), 3.74 (d, J=2.4 Hz, 1H), 3.68 (s, 2H), 3.57-3.48 (m, 2H), 3.40-3.33 (m, 2H), 3.17-3.05 (m, 1H), 2.99-2.92 (m, 2H), 2.86 (d, J=0.4 Hz, 1H), 2.70-2.59 (m, 1H), 2.25-2.11 (m, 2H), 1.93-1.52 (m, 14H), 1.50-1.37 (m, 13H), 1.20-1.12 (m, 4H), 1.10-0.95 (m, 4H). ¹⁹F NMR (377 MHz, METHANOL-d₄), δ=−120.772.

### Step 4: Synthesis of Intermediate 1-7

To a solution of Intermediate 1-6 (1.8 g, 2.17 mmol, 1 eq) in DCM (6 mL) was added HCl/dioxane (2 M, 12 mL). The mixture was stirred at 25° C. for 1 hr. The reaction liquid was concentrated under reduced pressure to obtain the crude product. The crude product was used in the next step without further purification. Intermediate 1-7 (1.6 g, crude, HCl salt) was obtained as a white solid. LCMS (Method D): Rt=0.278 min, [M+H]⁺=728.4.

### Step 5: Synthesis of Intermediate 1-9

To a solution of Intermediate 1-8 (300 mg, 614.10 μmol, 1 eq) in DMF (5 mL) was added HOAt (83.59 mg, 614.10 μmol, 85.90 μL, 1 eq), EDCI (353.17 mg, 1.84 mmol, 3 eq), NMM (310.57 mg, 3.07 mmol, 337.58 μL, 5 eq) and Intermediate 1-7 (563.28 mg, 736.92 μmol, 1.2 eq, HCl salt). The mixture was stirred at 25° C. for 1 hr. The residue was diluted with H₂O (20 mL) and extracted with EA (10 mL*3), dried over Na₂SO₄, filtered and concentrated under reduced pressure to give a residue. The crude product was purified by reversed-phase chromatography (0.1% NH₃·H₂O). The eluent was concentrated in vacuum to remove ACN, then it was lyophilized. Intermediate 1-9 (400 mg, 310.65 μmol, 50.59% yield, 93.07% purity) was obtained as a white solid. LCMS (Method D): Rt=0.467 min, [M+H]⁺=1198.6. SFC: Rt=0.710 min. ¹H NMR (400 MHz, METHANOL-d4) δ=8.37 (d, J=7.6 Hz, 1H), 7.95 (d, J=7.2 Hz, 1H), 7.90-7.80 (m, 2H), 7.78-7.67 (m, 2H), 7.52-7.35 (m, 5H), 7.16 (t, J=8.8 Hz, 1H), 7.00-6.89 (m, 2H), 4.94 (t, J=7.6 Hz, 1H), 4.55 (d, J=12.4 Hz, 3H), 4.48-4.41 (m, 1H), 4.39 (s, 2H), 4.28 (d, J=12.8 Hz, 1H), 4.22 (s, 2H), 3.94-3.82 (m, 1H), 3.81-3.64 (m, 4H), 3.59-3.45 (m, 2H), 3.27-3.23 (m, 1H), 3.22-3.07 (m, 3H), 2.97-2.83 (m, 2H), 2.80-2.61 (m, 3H), 2.13-2.04 (m, 2H), 2.04 (s, 1H), 1.96-1.81 (m, 5H), 1.81-1.57 (m, 10H), 1.51-1.37 (m, 10H), 1.34-1.00 (m, 12H). ¹⁹F NMR (377 MHz, METHANOL-d₄), δ=−113.43 (s, 1F), −115.49 (s, 1F), −120.76 (d, J=28.3 Hz, 1F).

### Step 6: Synthesis of I-719

To a solution of Intermediate 1-9 (400 mg, 333.77 μmol, 1 eq) in DCM (3 mL) was added TFA (1.54 g, 13.46 mmol, 1 mL, 40.33 eq). The mixture was stirred at 25° C. for 1 hr. The reaction liquid was concentrated under reduced pressure to obtain the crude product. The crude product was purified by reversed-phase HPLC (column: CD02-Waters Xbidge BEH C18 150*25*10 um; mobile phase: [water (NH₄HCO₃)-ACN]; gradient: 39%-69% B over 10 min). The eluent was concentrated in vacuum to remove ACN. Then it was lyophilized. I-719 (228.11 mg, 206.03 μmol, 61.73% yield, 99.2% purity) was obtained as a white solid. LCMS (Method H): Rt=0.722 min, [M+H]⁺=1098.8. SFC: Rt=0.1.830 min. ¹H NMR (400 MHz, METHANOL-d4) δ=8.39-8.34 (m, 1H), 7.98-7.92 (m, 1H), 7.90-7.80 (m, 2H), 7.78-7.74 (m, 1H), 7.73-7.67 (m, 1H), 7.53-7.41 (m, 4H), 7.41-7.33 (m, 1H), 7.16 (t, J=9.2 Hz, 1H), 6.99-6.88 (m, 2H), 4.99-4.90 (m, 1H), 4.61-4.49 (m, 2H), 4.38 (s, 2H), 4.32-4.23 (m, 1H), 3.89-3.77 (m, 4H), 3.76-3.62 (m, 3H), 3.57 (s, 1H), 3.54-3.41 (m, 3H), 3.24 (d, J=4.8 Hz, 1H), 3.20-3.07 (m, 3H), 2.95-2.78 (m, 3H), 2.77-2.63 (m, 2H), 2.12-1.98 (m, 3H), 1.94-1.81 (m, 5H), 1.81-1.54 (m, 10H), 1.46-0.95 (m, 13H). ¹⁹F NMR (377 MHz, METHANOL-d4) δ=−113.25-−113.56 (m, 1F), −116.20-−116.33 (m, 1F), −120.71-−120.82 (m, 1F).

### Step 7: Synthesis of Intermediate 1-12

To a solution of Intermediate 1-11 (1.5 g, 4.98 mmol, 1 eq) in DMF (15 mL) was added HOAt (677.65 mg, 4.98 mmol, 696.46 μL, 1 eq), EDCI (2.86 g, 14.94 mmol, 3 eq), and NMM (2.52 g, 24.89 mmol, 2.74 mL, 5 eq), followed by Intermediate 1-10 (1.27 g, 4.98 mmol, 1 eq, HCl salt). The mixture was stirred at 25° C. for 1 hr. The reaction mixture was diluted with water (15 mL) and extracted with EA (10 mL*3). The combined organic layers were dried over Na₂SO₄, filtered and concentrated under reduced pressure to give a residue. The residue was purified by flash silica gel chromatography (ISCO; 40 g SepaFlash Silica Flash Column, Eluent of 0˜100% Petroleum ether/Ethyl acetate @60 mL/min). The eluent was concentrated under reduced pressure to give Intermediate 1-12 (2.4 g, 4.78 mmol, 95.92% yield) as a colorless oil. LCMS (Method D): Rt=0.516 min, [M+H]⁺=503.2. SFC: Rt=1.122 min.

### Step 8: Synthesis of Intermediate 1-8

To a solution of Intermediate 1-12 (2.4 g, 4.78 mmol, 1 eq) in MeOH (8 mL), H₂O (8 mL) and THF (8 mL) was added LiOH·H₂O (400.81 mg, 9.55 mmol, 2 eq). The mixture was stirred at 25° C. for 3 hr. The mixture was adjusted to pH=5 with citric acid and extracted with EA (10 mL*3). The combined organic layers were dried over Na₂SO₄, filtered and concentrated under reduced pressure to give a residue. The residue was used for the next step without further purification. Intermediate 1-8 (2.3 g, 4.71 mmol, 98.58% yield) was obtained as a colorless oil. LCMS (Method D): Rt=0.469 min, [M-Boc+H]⁺=389.1. SFC: Rt=0.836 min.

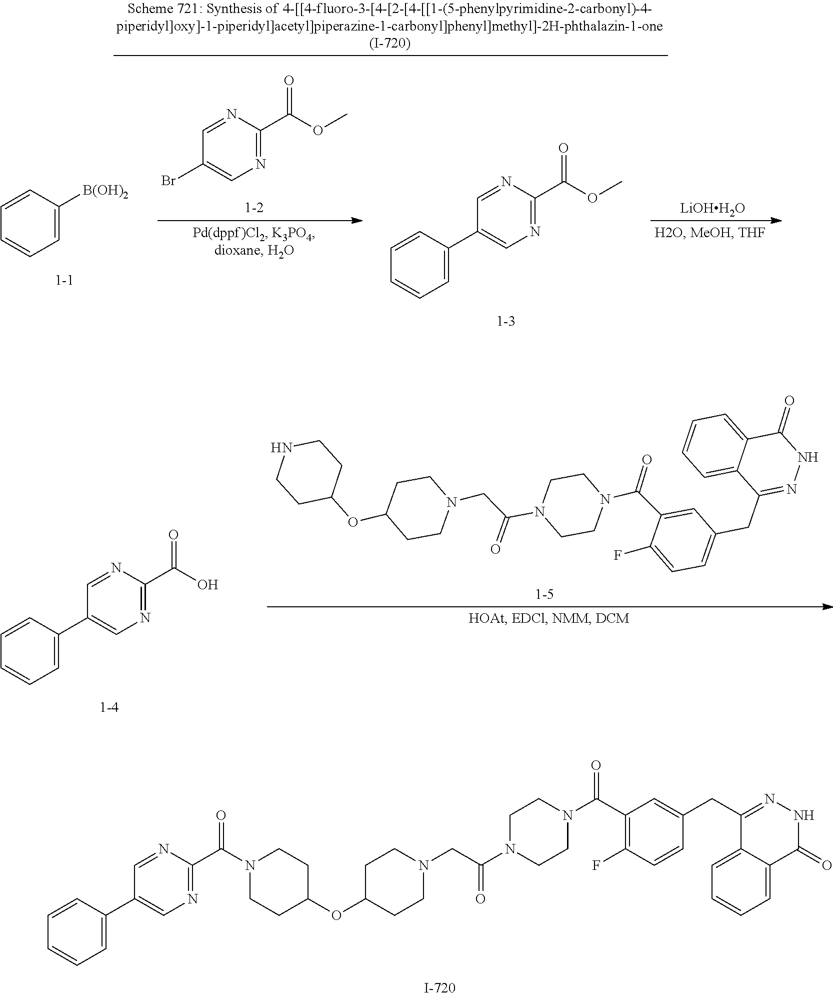

### Step 1: Synthesis of Intermediate 1-3

To a solution of Intermediate 1-1 (1.5 g, 12.30 mmol, 1 eq) and Intermediate 1-2 (2.67 g, 12.30 mmol, 1 eq) in H₂O (15 mL) and dioxane (60 mL) was added cyclopentyl (diphenyl) phosphane; dichloropalladium; iron (450.08 mg, 615.11 μmol, 0.05 eq) and K₂CO₃ (5.10 g, 36.91 mmol, 3 eq). The suspension was degassed and purged with N₂ 3 times. The mixture was stirred at 80° C. for 1 hr. The mixture was concentrated to give a residue. The reaction mixture was diluted with water (120 mL). Then the mixture was adjusted to pH=6-7 with a saturated hydrochloric acid aqueous solution. Then the reaction mixture was extracted with DCM (40 mL×3). The combined organic layers were washed with brine (40 mL×3) and dried over sodium sulfate, filtered and concentrated under reduced pressure to give Intermediate 1-3 (1.6 g, 7.47 mmol, 60.71% yield) as a white solid. LCMS (Method D): Rt: 0.262 min, [M+H]⁺=201.1.

### Step 2: Synthesis of Intermediate 1-4

To a solution of Intermediate 1-3 (1.5 g, 7.00 mmol, 1 eq) in H₂O (4 mL), MeOH (8 mL) and THF (8 mL) was added LiOH·H₂O (881.51 mg, 21.01 mmol, 3 eq). The mixture was stirred at 25° C. for 0.5 hr. The mixture was concentrated to give a residue. The reaction mixture was diluted with water (120 mL). Then the mixture was adjusted to pH=6-7 with a saturated hydrochloric acid aqueous solution. Then the reaction mixture was extracted with DCM (120 mL×3). The combined organic layers were washed with brine (120 mL×3) and dried over sodium sulfate, filtered and concentrated under reduced pressure to give Intermediate 1-4 (1.35 g, 6.74 mmol, 96.31% yield) as a brown solid. LCMS (Method H): Rt=0.338 min, [M+H]⁺=201.1. ¹H NMR (400 MHz, METHANOL-d4) δ=9.21 (s, 2H), 7.78 (d, J=6.8 Hz, 2H), 7.63-7.47 (m, 3H).

### Step 3: Synthesis of I-720

To a solution of Intermediate 1-4 (200 mg, 999.04 μmol, 1 eq) in DCM (8 mL) was added EDCI (574.55 mg, 3.00 mmol, 3 eq), NMM (505.26 mg, 5.00 mmol, 549.20 μL, 5 eq) and HOAt (135.98 mg, 999.04 μmol, 139.75 μL, 1 eq) at 25° C. for 0.5 hr. Then Intermediate 1-5 (590.12 mg, 999.04 μmol, 1 eq) was added into the mixture and it was stirred at 25° C. for 0.5 hr. The reaction mixture was diluted with water (60 mL) and extracted with ethyl acetate (20 mL×3). The combined organic layers were washed with brine (20 mL×3) and dried over sodium sulfate, filtered and concentrated under reduced pressure to give a residue. The crude product was purified by reversed-phase chromatography (Neutral condition) and the eluent was concentrated under reduced pressure to remove ACN and then lyophilized to give I-720 (450.26 mg, 582.59 μmol, 58.31% yield, 100% purity) as a white solid. LCMS (Method D): Rt=0.333 min, [M+H]⁺=773.1. ¹H NMR (400 MHz, METHANOL-d4) δ=9.14 (s, 2H), 8.39-8.33 (m, 1H), 7.99-7.92 (m, 1H), 7.91-7.79 (m, 2H), 7.79-7.74 (m, 2H), 7.60-7.44 (m, 4H), 7.38 (s, 1H), 7.18-7.16 (m, 1H), 4.38 (s, 2H), 4.09-3.99 (m, 1H), 3.87-3.69 (m, 4H), 3.68-3.47 (m, 6H), 3.33 (s, 1H), 3.29-3.12 (m, 4H), 2.77 (s, 2H), 2.35-2.18 (m, 2H), 2.01-1.81 (m, 4H), 1.75-1.49 (m, 4H). ¹⁹F NMR (376 MHz, METHANOL-d4) δ=−120.764.

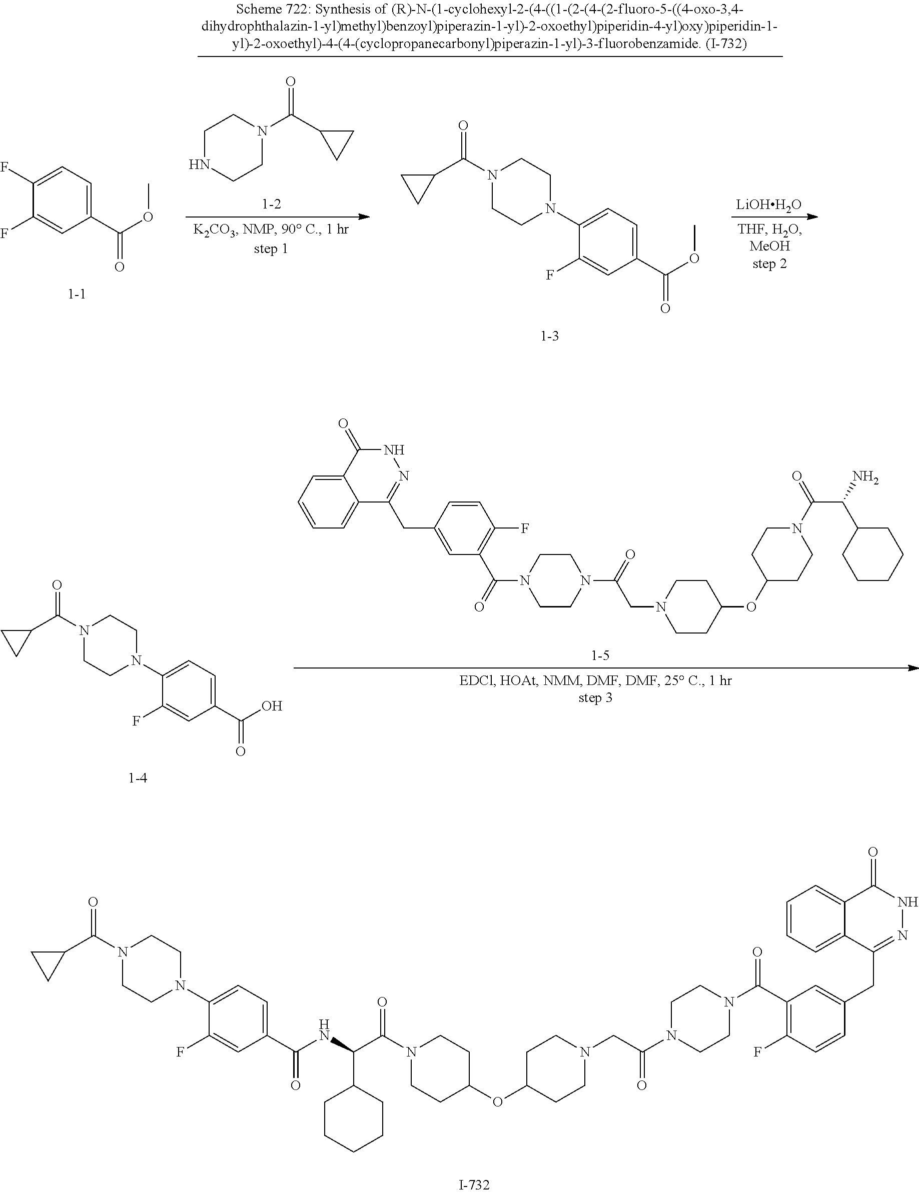

### Step 1: Synthesis of Intermediate 1-3.

To a solution of Intermediate 1-1 (100 mg, 580.96 μmol, 1 eq) and Intermediate 1-2 (89.59 mg, 580.96 μmol, 1 eq) in NMP (1 mL) was added K₂CO₃ (240.88 mg, 1.74 mmol, 3 eq). The mixture was stirred at 90° C. for 1 hr. To the reaction mixture was added H₂O (2 mL) and then it was extracted with DCM (2 mL*3), the combined organic phase was washed, dried by Na₂SO₄, filtered and concentrated. The residue was purified by flash silica gel chromatography (ISCO; 12 g SepaFlash Silica Flash Column, Eluent of 0-100% Ethyl acetate/Petroleum ethergradient @30 mL/min) and the eluent was concentrated to give Intermediate 1-3 (60 mg, 195.87 μmol, 33.71% yield) as a white solid. LCMS (Method D): Retention time: 0.439 min, [M+H]⁺=307.1. ¹H NMR (400 MHz, METHANOL-d4) δ=7.82-7.71 (m, 1H), 7.70-7.62 (m, 1H), 7.09 (t, J=8.8 Hz, 1H), 3.95 (s, 2H), 3.88 (s, 3H), 3.77 (s, 2H), 3.19 (s, 4H), 2.00 (s, 1H), 0.93-0.88 (m, 2H), 0.85 (s, 2H).

### Step 2: Synthesis of Intermediate 1-4.

To a solution of Intermediate 1-3 (50 mg, 163.22 μmol, 1 eq) in THF (0.2 mL), MeOH (0.2 mL) and H₂O (0.2 mL) was added LiOH·H₂O (13.70 mg, 326.44 μmol, 2 eq). The mixture was stirred at 25° C. for 1 hr. The aqueous phase was adjusted to pH=7 with 1 N aq·HCl and then extracted with DCM (10 mL*3), the combined organic phase was washed, dried by Na₂SO₄, filtered and concentrated. The crude was used in the next step. Intermediate 1-4 (40 mg, 136.84 μmol, 83.84% yield) was obtained as a white solid. LCMS (Method D): Retention time: 0.305 min, [M+H]⁺=293.1.

### Step 3: Synthesis of I-732.

To a solution of Intermediate 1-4 (30 mg, 102.63 μmol, 1 eq) and Intermediate 1-5 (74.91 mg, 97.75 μmol, 9.52e−1 eq, HCl salt) in DCM (0.3 mL) was added EDCI (39.35 mg, 205.27 μmol, 2 eq), HOAt (13.97 mg, 102.63 μmol, 14.36 μL, 1 eq) and NMM (51.90 mg, 513.16 μmol, 56.42 μL, 5 eq). The mixture was stirred at 25° C. for 1 hr. To the reaction mixture was added H₂O (2 mL) and then it was extracted with DCM (2 mL*3), the combined organic phase was washed, dried by Na₂SO₄, filtered and concentrated. The crude product was purified by reversed phase chromatography (0.1% FA condition), the eluent was concentrated to remove ACN and lyophilized to get I-732 (24.91 mg, 22.05 μmol, 21.49% yield, 92.9% purity, FA salt) as a white solid. LCMS (Method D): Retention time: 0.386 min, [M+H]⁺=1004.6. SFC: Retention time: 1.322 min. ¹H NMR (400 MHz, METHANOL-d4) δ=8.53 (s, 1H), 8.38 (d, J=7.2 Hz, 1H), 8.00-7.92 (m, 1H), 7.92-7.79 (m, 2H), 7.70-7.55 (m, 2H), 7.50 (d, J=3.6 Hz, 1H), 7.38 (d, J=4.8 Hz, 1H), 7.17 (t, J=8.8 Hz, 1H), 7.09 (t, J=8.4 Hz, 1H), 4.91 (d, J=4.4 Hz, 1H), 4.39 (s, 2H), 4.09-4.00 (m, 1H), 3.95 (s, 3H), 3.85-3.61 (m, 7H), 3.61-3.39 (m, 5H), 3.35-3.33 (m, 1H), 3.24 (d, J=3.2 Hz, 4H), 3.18-3.10 (m, 2H), 2.87-2.69 (m, 2H), 2.44-2.20 (m, 2H), 2.06-1.99 (m, 1H), 1.98-1.81 (m, 6H), 1.78 (d, J=11.2 Hz, 2H), 1.72-1.66 (m, 2H), 1.66-1.54 (m, 3H), 1.54-1.43 (m, 1H), 1.34-1.22 (m, 3H), 1.16-1.02 (m, 2H), 0.94-0.88 (m, 2H), 0.88-0.82 (m, 2H). ¹⁹F NMR (377 MHz, METHANOL-d₄), δ=−120.71-−120.81 (m, 1F), −123.32-−123.43 (m, 1F).

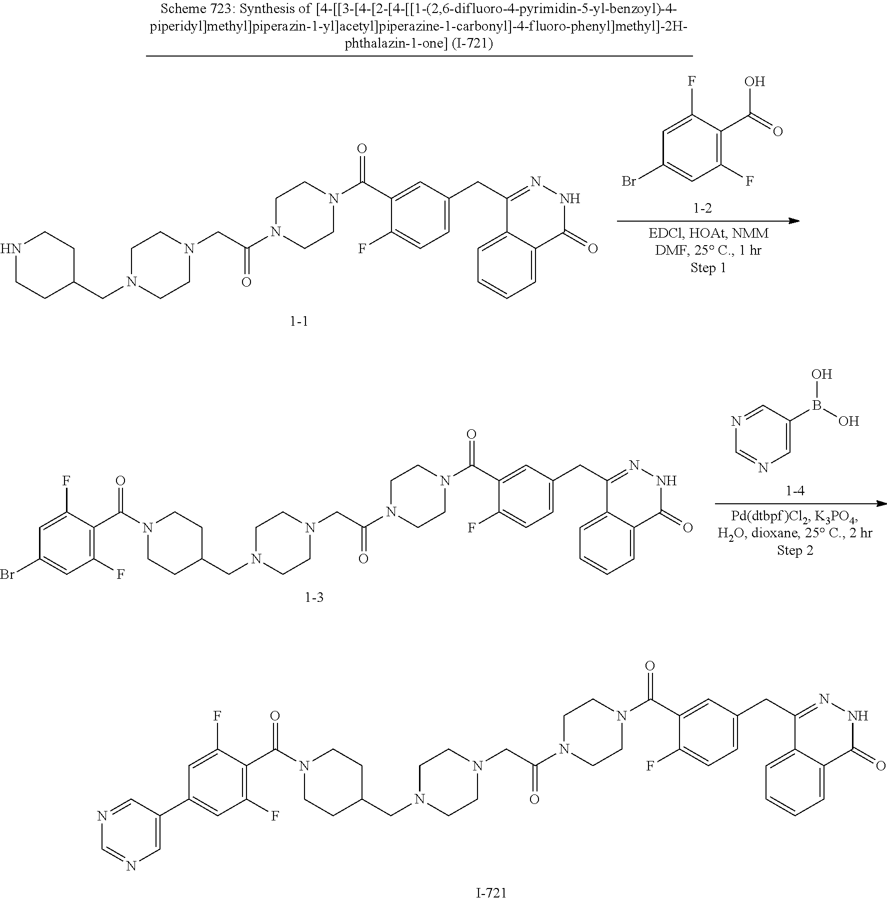 [SMILES: O=C(CN1CCN(CC2CCNCC2)CC1)N1CCN(C(=O)c2cc(Cc3n[nH]c(=O)c4ccccc34)ccc2F)CC1 ; O=C(O)c1c(F)cc(Br)cc1F ; O=C(CN1CCN(CC2CCN(C(=O)c3c(F)cc(Br)cc3F)CC2)CC1)N1CCN(C(=O)c2cc(Cc3n[nH]c(=O)c4ccccc34)ccc2F)CC1 ; OB(O)c1cncnc1 ; O=C(CN1CCN(CC2CCN(C(=O)c3c(F)cc(-c4cncnc4)cc3F)CC2)CC1)N1CCN(C(=O)c2cc(Cc3n[nH]c(=O)c4ccccc34)ccc2F)CC1]

### Step 1: Synthesis of Intermediate 1-3.

To a solution of Intermediate 1-1 (1 g, 1.60 mmol, 1 eq, HCl salt) and Intermediate 1-2 (378.49 mg, 1.60 mmol, 1 eq) in DMF (10 mL) was added EDCI (918.46 mg, 4.79 mmol, 3 eq), HOAt (217.37 mg, 1.60 mmol, 223.41 μL, 1 eq) and NMM (807.67 mg, 7.99 mmol, 877.91 μL, 5 eq). The mixture was stirred at 25° C. for 1 h. The reaction mixture was diluted with H₂O (20 mL) and extracted with DCM (20 mL*3). The combined organic layers were washed with aq. NaCl (20 mL), dried over Na₂SO₄, filtered and concentrated under reduced pressure to give a residue. The residue was purified by reversed phase chromatography (0.1% FA), and the eluent was concentrated to remove ACN and lyophilized to give product. Intermediate 1-3 (1.1 g, 1.36 mmol, 85.17% yield) was obtained as a white solid. LCMS (Method D): Rt=0.324 min, [M+H]⁺=808.1.

### Step 2: Synthesis of I-721.

A mixture of Intermediate 1-3 (70 mg, 86.56 μmol, 1 eq), Intermediate 1-4 (10.73 mg, 86.56 μmol, 1 eq), ditert-butyl(cyclopenta-1,4-dien-1-yl)phosphane; dichloropalladium;iron (5.64 mg, 8.66 μmol, 0.1 eq), and K₃PO₄ (55.12 mg, 259.68 μmol, 3 eq) in dioxane (1 mL) and H₂O (0.2 mL) was degassed and purged with N₂ 3 times, and then the mixture was stirred at 80° C. for 2 h under N₂ atmosphere. The mixture was quenched by addition of H₂O (1.2 mL) at 25° C. and extracted with DCM (0.4 mL*3), the combined organic phase was dried over Na₂SO₄, filtered and concentrated under reduced pressure to give a crude product. The crude product was purified by prep-HPLC (column: CD02-Waters Xbidge BEH C18 150*25*10 um; mobile phase: [water (NH₃H₂O)-ACN]; gradient: 19%-49% B over 11 min), the eluent was concentrated to remove ACN and lyophilized to give the product. I-721 (17.67 mg, 19.17 μmol, 22.14% yield, 100% purity, TFA salt) was obtained as a white solid. LCMS (Method D): Rt=0.314 min, [M+H]⁺=808.3. ¹H NMR (400 MHz, METHANOL-d₄) δ=9.21 (s, 1H), 9.14 (s, 2H), 8.39-8.35 (m, 1H), 7.98-7.93 (m, 1H), 7.90-7.81 (m, 2H), 7.59-7.56 (m, 2H), 7.50 (d, J=2.4 Hz, 1H), 7.39-7.35 (m, 1H), 7.18-7.14 (m, 1H), 4.70 (d, J=12.8 Hz, 1H), 4.39 (s, 2H), 3.82-3.49 (m, 8H), 3.28-3.12 (m, 2H), 2.96-2.90 (m, 1H), 2.83-2.24 (m, 11H), 1.95 (d, J=8.0 Hz, 2H), 1.82 (d, J=12.0 Hz, 1H), 1.40-1.05 (m, 3H). ¹⁹F NMR (376 MHz, METHANOL-d₄) δ=−76.96 (s, 1F), −113.94 (br dd, J=5.7, 22.6 Hz, 2F), −120.75 (br d, J=28.3 Hz, 1F).

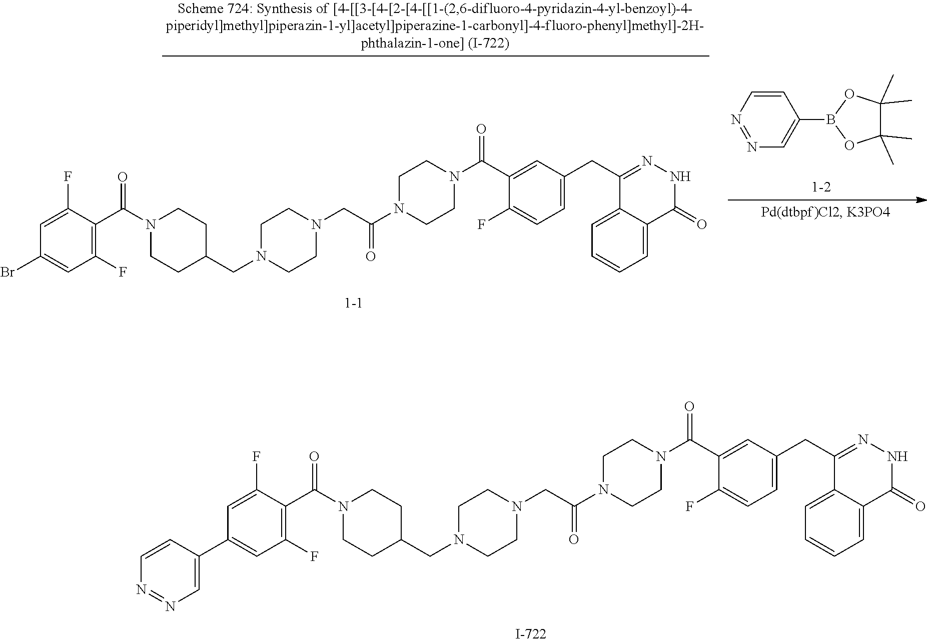 [SMILES: O=C(CN1CCN(CC2CCN(C(=O)c3c(F)cc(Br)cc3F)CC2)CC1)N1CCN(C(=O)c2cc(Cc3n[nH]c(=O)c4ccccc34)ccc2F)CC1 ; CC1(C)OB(c2ccnnc2)OC1(C)C ; O=C(CN1CCN(CC2CCN(C(=O)c3c(F)cc(-c4ccnnc4)cc3F)CC2)CC1)N1CCN(C(=O)c2cc(Cc3n[nH]c(=O)c4ccccc34)ccc2F)CC1]

To a solution of intermediate 1-1 (70 mg, 86.56 μmol, 1 eq) and intermediate 1-2 (17.84 mg, 86.56 μmol, 1 eq) in dioxane (0.5 mL) and H₂O (0.1 mL) was added K₃PO₄ (55.12 mg, 259.68 μmol, 3 eq) and N-[[1-(2-thienylmethyl)-4-piperidyl]methyl]-3-(trifluoromethyl)benzamide (6.62 mg, 17.31 μmol, 0.2 eq) and the mixture was stirred at 80° C. for 1 hr. Then the mixture was washed with water (1 mL) and extracted with DCM (1 mL*3), the combined organic phase was dried by Na₂SO₄, and concentrated under reduced pressure to give a residue. The crude was purified by prep-HPLC (column: CD01-Phenomenex luna C18 150*25*10 um; mobile phase: [water(FA)-ACN]; gradient: 33%-33% B over 10 min) and the eluent was concentrated to remove ACN and lyophilized to give product. I-722 (25 mg, 29.22 μmol, 33.75% yield, 99.796% purity, FA salt) was obtained as a yellow solid. LCMS (Method D): Retention time: 0.307 min, [M+H]⁺=808.2. ¹H NMR (400 MHz, METHANOL-d₄) δ=¹H NMR (400 MHz, METHANOL-d4) δ=9.59 (d, J=1.2 Hz, 1H), 9.29 (d, J=5.6 Hz, 1H), 8.53-8.26 (m, 2H), 8.10-8.02 (m, 1H), 7.98-7.91 (m, 1H), 7.90-7.80 (m, 2H), 7.74-7.64 (m, 2H), 7.49 (s, 1H), 7.41-7.32 (m, 1H), 7.16 (t, J=8.8 Hz, 1H), 4.70 (d, J=13.2 Hz, 1H), 4.38 (s, 2H), 3.83-3.72 (m, 2H), 3.69-3.57 (m, 3H), 3.52 (s, 3H), 3.45 (s, 1H), 3.34 (d, J=3.6 Hz, 1H), 3.23 (t, J=12.8 Hz, 2H), 3.02-2.72 (m, 9H), 2.63-2.53 (m, 2H), 2.10-1.92 (m, 2H), 1.84 (d, J=12.8 Hz, 1H), 1.31-1.16 (m, 2H). ¹⁹F NMR (377 MHz, METHANOL-d4), δ=−113.16-−113.85 (m, 2F), −120.69 (d, J=33.6 Hz, 1F).

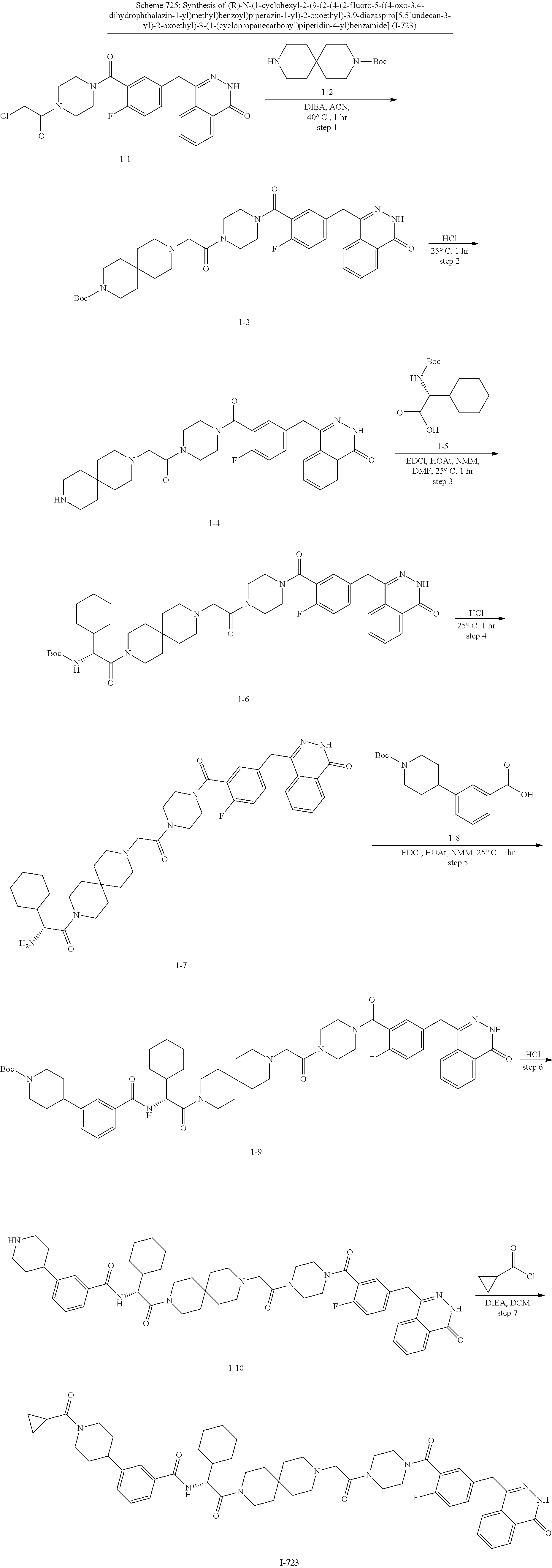

### Step 1: Synthesis of Intermediate 1-3.

To a solution of Intermediate 1-1 (8 g, 18.06 mmol, 1 eq) and Intermediate 1-2 (5.05 g, 19.87 mmol, 1.1 eq) in ACN (80 mL) was added DIEA (4.67 g, 36.13 mmol, 6.29 mL, 2 eq). The mixture was stirred at 40° C. for 1 hr. To the reaction mixture was added H₂O (100 mL) and then it was extracted with DCM (100 mL*3), the combined organic phase was washed, dried by Na₂SO₄, filtered and concentrated. The residue was purified by flash silica gel chromatography (ISCO; 120 g SepaFlash Silica Flash Column, Eluent of 0-100% Ethyl acetate/Petroleum ethergradient @50 mL/min) and the eluent was concentrated to give product. Intermediate 1-3 (15 g, crude) was obtained as a white solid. LCMS (Method D): Retention time: 0.315 min, [M+H]⁺=661.6.

### Step 2: Synthesis of Intermediate 1-4.

A solution of Intermediate 1-3 (14 g, 21.19 mmol, 1 eq) in HCl/dioxane (2 M, 140.00 mL, 13.22 eq) was stirred at 25° C. for 1 hr. The reaction mixture was concentrated under reduced pressure to give a residue. The crude was used in the next step. Intermediate 1-4 (15 g, crude, HCl salt) was obtained as a white solid. LCMS (Method D): Retention time: 0.196 min, [M+H]⁺=561.3.

### Step 3: Synthesis of Intermediate 1-6.

To a solution of Intermediate 1-4 (14 g, 23.45 mmol, 1 eq, HCl salt) and Intermediate 1-5 (7.24 g, 28.13 mmol, 1.2 eq) in DMF (140 mL) was added EDCI (8.99 g, 46.89 mmol, 2 eq), HOAt (3.19 g, 23.45 mmol, 3.28 mL, 1 eq) and NMM (11.86 g, 117.23 mmol, 12.89 mL, 5 eq). The mixture was stirred at 25° C. for 1 hr. To the reaction mixture was added H₂O (200 mL) and then it was extracted with DCM (200 mL*3), the combined organic phase was washed, dried by Na₂SO₄, filtered and concentrated. The residue was purified by flash silica gel chromatography (ISCO; 120 g SepaFlash Silica Flash Column, Eluent of 0˜100% Ethyl acetate/Petroleum ethergradient @100 mL/min) and the eluent was concentrated to give product. Intermediate 1-6 (14 g, 16.45 mmol, 70.16% yield, 94% purity) was obtained as a white solid. LCMS (Method D): Retention time: 0.359 min, [M+H]⁺=800.6. SFC: Retention time: 1.357 min. Step 4: Synthesis of Intermediate 1-7.

A solution of Intermediate 1-6 (13 g, 16.25 mmol, 1 eq) in HCl/dioxane (2 M, 130.00 mL, 16.00 eq) was stirred at 25° C. for 0.5 hr. The reaction mixture was concentrated under reduced pressure to give a residue. The crude was used in the next step. Intermediate 1-7 (16 g, crude, HCl salt) was obtained as a white solid. LCMS (Method D): Retention time: 0.253 min, [M+H]⁺=700.4 Step 5: Synthesis of Intermediate 1-9.

To a solution of Intermediate 1-7 (1.8 g, 2.44 mmol, 1 eq, HCl salt) and Intermediate 1-8 (746.50 mg, 2.44 mmol, 1 eq) in DMF (18 mL) was added EDCI (468.63 mg, 2.44 mmol, 1 eq) and HOAt (332.73 mg, 2.44 mmol, 341.97 μL, 1 eq) and NMM (247.27 mg, 2.44 mmol, 268.77 μL, 1 eq). The mixture was stirred at 25° C. for 1 hr. To the reaction mixture was added H₂O (50 mL) and then it was extracted with DCM (50 mL*3), and the combined organic phase was washed, dried by Na₂SO₄, filtered and concentrated. The residue was purified by flash silica gel chromatography (ISCO; 40 g SepaFlash Silica Flash Column, Eluent of 0˜100% Ethyl acetate/Petroleum ethergradient @50 mL/min) and the eluent was concentrated to give Intermediate 1-9 (2.2 g, 2.23 mmol, 91.16% yield) as a white solid. LCMS (Method D): Retention time: 0.398 min, [M+H]⁺=988.0.

### Step 6: Synthesis of Intermediate 1-10.

A solution of Intermediate 1-9 (2 g, 2.03 mmol, 1 eq) in HCl/dioxane (2 M, 20.00 mL, 19.74 eq) was stirred at 25° C. for 1 hr. The reaction mixture was concentrated under reduced pressure to give a residue. The residue was purified by prep-HPLC (column: CD06-Waters Xbidge C18 150*40*10 um; mobile phase: [water (HCl)-ACN]; gradient: 12%-42% B over 11 min) and the eluent was concentrated to remove ACN and lyophilized to give Intermediate 1-10 (2 g, crude, HCl salt) as a white solid. LCMS (Method D): Retention time: 0.313 min, [M+H]⁺=887.5. SFC: Retention time: 1.194 min, 1.513 min.

### Step 7: Synthesis of I-723.

To a solution of Intermediate 1-10 (1 g, 1.08 mmol, 1 eq, HCl salt) and cyclopropanecarbonyl chloride (135.83 mg, 1.30 mmol, 117.90 μL, 1.2 eq) in DCM (10 mL) was added DIEA (279.88 mg, 2.17 mmol, 377.20 μL, 2 eq). The mixture was stirred at 25° C. for 1 hr. To the reaction mixture was added H₂O (15 mL) and then it was extracted with DCM (15 mL*3), the combined organic phase was washed, dried by Na₂SO₄, filtered and concentrated. The residue was purified by prep-HPLC (column: CD05-Phenomenex luna C18 150*40*10 um; mobile phase: [water (FA)-ACN]; gradient: 15%-45% B over 10 min) and the eluent was concentrated to remove ACN and lyophilized to give product. I-723 (178.78 mg, 175.18 μmol, 16.18% yield, 98.105% purity, FA salt) was obtained as a yellow solid. LCMS (Method D): Retention time: 0.426 min, [M+H]⁺=955.6. SFC: Retention time: 0.676 min, 1.150 min ¹H NMR (400 MHz, METHANOL-d4) δ=8.48 (s, 1H), 8.37 (d, J=7.6 Hz, 1H), 7.99-7.92 (m, 1H), 7.91-7.79 (m, 2H), 7.73 (s, 1H), 7.68 (d, J=7.6 Hz, 1H), 7.55-7.33 (m, 4H), 7.17 (t, J=9.2 Hz, 1H), 4.92 (d, J=9.2 Hz, 1H), 4.67 (d, J=13.2 Hz, 1H), 4.54-4.44 (m, 1H), 4.39 (s, 2H), 3.87-3.72 (m, 4H), 3.71-3.57 (m, 4H), 3.56-3.45 (m, 4H), 3.39-3.32 (m, 2H), 3.26 (s, 1H), 2.98-2.88 (m, 1H), 2.85-2.59 (m, 5H), 2.06-1.95 (m, 2H), 1.89 (t, J=10.4 Hz, 3H), 1.83-1.72 (m, 4H), 1.72-1.56 (m, 8H), 1.51 (d, J=4.0 Hz, 2H), 1.35-1.20 (m, 3H), 1.18-1.04 (m, 2H), 0.95-0.86 (m, 2H), 0.86-0.76 (m, 2H). ¹⁹F NMR (377 MHz, METHANOL-d₄), δ=−120.48-−120.99 (m, 1F).

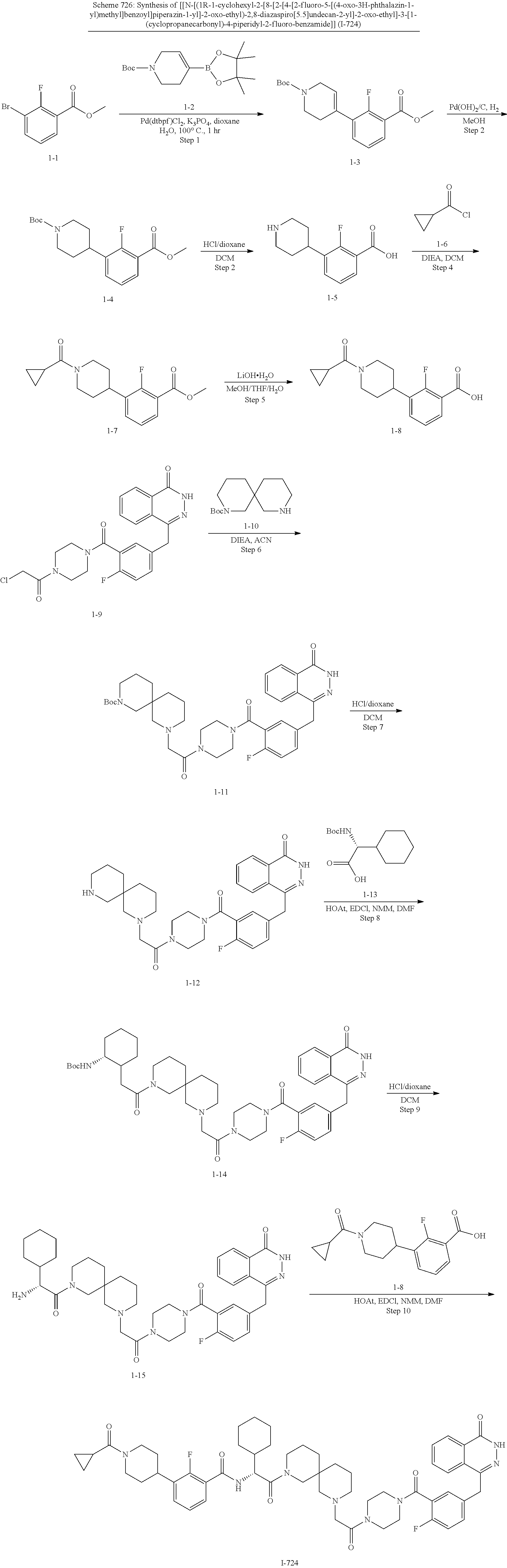

### Step 1: Synthesis of Intermediate 1-3

To a solution of Intermediate 1-2 (796.13 mg, 2.57 mmol, 1 eq) in dioxane (6 mL) and H₂O (1.2 mL) was added Intermediate 1-1 (600 mg, 2.57 mmol, 1 eq), K₃PO₄ (1.64 g, 7.72 mmol, 3 eq), and ditert-butyl(cyclopenta-1,4-dien-1-yl)phosphane;dichloropalladium;iron (167.81 mg, 257.47 μmol, 0.1 eq). The mixture was stirred at 100° C. for 1 hr under N₂. The reaction mixture diluted with H₂O (5 mL) and extracted with EA (5 mL*2). The combined organic layers were dried over Na₂SO₄, filtered and concentrated under reduced pressure to give a residue. The residue was purified by flash silica gel chromatography (ISCO; 20 g SepaFlash Silica Flash Column, Eluent of 0˜10% Ethylacetate/Petroleum ether gradient @60 mL/min). The eluent was concentrated under reduced pressure to give Intermediate 1-3 (940 mg, crude) as a yellow oil. LCMS (Method D): Rt: 0.481 min, [M+Na]⁺=358.1. ¹H NMR (400 MHz, METHANOL-d4) δ=7.82-7.76 (m, 1H), 7.55-7.49 (m, 1H), 7.26-7.20 (m, 1H), 5.94 (s, 1H), 4.07 (s, 2H), 3.90 (s, 3H), 3.69-3.56 (m, 2H), 2.55-2.42 (m, 2H), 1.50 (s, 9H). ¹⁹F NMR (377 MHz, METHANOL-d4) δ=−115.233.

### Step 2: Synthesis of Intermediate 1-4

To a solution of Intermediate 1-3 (940 mg, 2.80 mmol, 1 eq) in MeOH (10 mL) was added Pd(OH)₂/C (500 mg, 20% purity). The flask was degassed and purged with H₂ 3 times, then the mixture was stirred at 25° C. for 1 hr under H₂ (15 psi) atmosphere. The mixture reaction was filtered and the filter cake was washed with MeOH (3 mL*3). The filtrate was concentrated under reduced pressure to give Intermediate 1-4 (900 mg, 2.48 mmol, 88.32% yield, 92.8% purity) as a yellow oil. LCMS (Method D): Rt: 0.481 min, [M+Na]=360.1.

### Step 3: Synthesis of Intermediate 1-5

A solution of Intermediate 1-4 (800 mg, 2.37 mmol, 1 eq) in DCM (4 mL) and HCl/dioxane (2 M, 8 mL) was stirred at 25° C. for 1 hr. The mixture was concentrated under reduced pressure to give Intermediate 1-5 (650 mg, crude, HCl salt) as a yellow solid. LCMS (Method D): Rt: 0.224 min, [M+H]⁺=238.1. ¹H NMR (400 MHz, METHANOL-d4) δ=7.85-7.78 (m, 1H), 7.60-7.52 (m, 1H), 7.32-7.23 (m, 1H), 3.91 (s, 3H), 3.56-3.48 (m, 2H), 3.30-3.26 (m, 1H), 3.24-3.14 (m, 2H), 2.11-1.97 (m, 4H).

### Step 4: Synthesis of Intermediate 1-7

To a solution of Intermediate 1-5 (650 mg, 2.37 mmol, 1 eq, HCl salt) in CH₂Cl₂ (8 mL) was added Intermediate 1-6 (297.87 mg, 2.85 mmol, 258.57 μL, 1.2 eq) and DIEA (920.70 mg, 7.12 mmol, 1.24 mL, 3 eq). The mixture was stirred at 0° C. for 0.5 hr. The mixture was diluted with H₂O (10 mL) and extracted with CH₂Cl₂ (10 mL*1). The combined organic layers were dried over Na₂SO₄, filtered and concentrated under reduced pressure to give a residue. The residue was purified by flash silica gel chromatography (ISCO; 20 g SepaFlash Silica Flash Column, Eluent of 0˜35% Ethyl acetate/Petroleum ether gradient @60 mL/min). The eluent was concentrated under reduced pressure to give Intermediate 1-7 (650 mg, crude, HCl salt) (570 mg, 1.84 mmol, 77.36% yield, 98.4% purity) as a colorless oil. LCMS (Method D): Rt: 0.405 min, [M+H]⁺=306.1.

### Step 5: Synthesis of Intermediate 1-8

To a solution of Intermediate 1-7 (470 mg, 1.54 mmol, 1 eq) in MeOH (1.5 mL), THF (1.5 mL) and H₂O (1.5 mL) was added LiOH·H₂O (193.76 mg, 4.62 mmol, 3 eq). The mixture was stirred at 25° C. for 1 hr. The mixture was diluted with H₂O (3 mL) and HCl (1M) was added to adjust pH to 4. The mixture was extracted with EA (3 mL*2). The combined organic layers were dried over Na₂SO₄, filtered and concentrated under reduced pressure to give Intermediate 1-8 (550 mg, crude) as a yellow oil. LCMS (Method D): Rt: 0.343 min, [M+H]⁺=292.1. ¹H NMR (400 MHz, METHANOL-d4) δ=7.80-7.74 (m, 1H), 7.55-7.49 (m, 1H), 7.25-7.18 (m, 1H), 4.74-4.62 (m, 1H), 4.55-4.42 (m, 1H), 3.30-3.22 (m, 2H), 2.85-2.72 (m, 1H), 2.01-1.92 (m, 2H), 1.91-1.83 (m, 1H), 1.81-1.60 (m, 2H), 0.92-0.80 (m, 4H). ¹⁹F NMR (377 MHz, METHANOL-d4) δ=−119.038.

### Step 6: Synthesis of Intermediate 1-11

To a solution of Intermediate 1-9 (700 mg, 1.58 mmol, 1 eq) in ACN (7 mL) was added Intermediate 1-10 (402.05 mg, 1.58 mmol, 1 eq) and DIEA (612.84 mg, 4.74 mmol, 825.93 μL, 3 eq). The mixture was stirred at 40° C. for 1 hr. The mixture was diluted with H₂O (10 mL) and extracted with EA (10 mL*2). The combined organic layers were dried over Na₂SO₄, filtered and concentrated under reduced pressure to give a residue. The residue was purified by flash silica gel chromatography (ISCO; 40 g SepaFlash Silica Flash Column, Eluent of 0-100% Ethylacetate/Petroleum ether@60 mL/min). The eluent was concentrated under reduced pressure to give Intermediate 1-11 (950 mg, 1.43 mmol, 90.19% yield, 99.15% purity) as a yellow solid. LCMS (Method D): Rt: 0.345 min, [M+H]⁺=661.2. ¹H NMR (400 MHz, METHANOL-d4) δ=8.41-8.33 (m, 1H), 8.00-7.92 (m, 1H), 7.91-7.80 (m, 2H), 7.53-7.33 (m, 2H), 7.21-7.11 (m, 1H), 4.43-4.33 (m, 2H), 4.02-3.49 (m, 8H), 3.01-2.81 (m, 1H), 2.68-2.52 (m, 1H), 2.29-2.04 (m, 2H), 1.67-1.40 (m, 11H), 1.29 (s, 5H), 1.17-1.04 (m, 1H). SFC: Rt=4.028 min, 5.423 min. ¹⁹F NMR (377 MHz, METHANOL-d4) δ=−120.802.

### Step 7: Synthesis of Intermediate 1-12

A solution of Intermediate 1-11 (100 mg, 151.34 μmol, 1 eq) in CH₂Cl₂ (0.5 mL) and HCl/dioxane (2 M, 0.5 mL) was stirred at 25° C. for 1 hr. The mixture was concentrated under reduced pressure to give Intermediate 1-12 (95 mg, crude, HCl salt) as a white solid. LCMS (Method D): Rt: 0.232 min, [M+H]⁺=561.2.

### Step 8: Synthesis of Intermediate 1-14

To a solution of Intermediate 1-13 (40.94 mg, 159.10 μmol, 1 eq) in DMF (1 mL) was added EDCI (91.50 mg, 477.29 μmol, 3 eq), HOAt (21.65 mg, 159.10 μmol, 22.26 μL, 1 eq), Intermediate 1-12 (95 mg, 159.10 μmol, 1 eq, HCl salt) and NMM (80.46 mg, 795.48 μmol, 87.46 μL, 5 eq). The mixture was stirred at 25° C. for 1 hr. The mixture was diluted with H₂O (1 mL) and extracted with EA (1.5 mL*2). The combined organic layers were washed with brine (1 mL*2), dried over Na₂SO₄, filtered and concentrated under reduced pressure to give a residue. The residue was purified by flash silica gel chromatography (ISCO; 12 g SepaFlash Silica Flash Column, Eluent of 0˜10% MeOH/Ethyl acetate gradient @60 mL/min). The eluent was concentrated under reduced pressure to give Intermediate 1-14 (100 mg, 124.13 μmol, 78.02% yield, 99.3% purity) as a yellow gum. LCMS (Method D): Rt: 0.400 mm, [M+H]⁺=800.4. SFC: Rt=4.251 min, 5.739 min.

### Step 9: Synthesis of Intermediate 1-15

A solution of Intermediate 1-14 (100 mg, 125.00 μmol, 1 eq) in DCM (0.5 mL) and HCl/dioxane (2 M) was stirred at 25° C. for 1 hr. The mixture was concentrated under reduced pressure to give Intermediate 1-15 (95 mg, crude, HCl salt) as a white solid. LCMS (Method D): Rt: 0.270 min, [M+H]⁺=700.3.

### Step 10: Synthesis of I-724

To a solution of Intermediate 1-8 (41.34 mg, 141.92 μmol, 1.1 eq) in DMF (1 mL) was added EDCI (74.20 mg, 387.06 μmol, 3 eq), HOAt (17.56 mg, 129.02 μmol, 18.05 μL, 1 eq), Intermediate 1-15 (95 mg, 129.02 μmol, 1 eq, HCl salt) and NMM (65.25 mg, 645.10 μmol, 70.92 μL, 5 eq). The mixture was stirred at 25° C. for 1 hr. The mixture was filtered and the filtrate was purified by reversed-phase chromatography (0.1% FA condition). The eluent was concentrated under reduced pressure to remove ACN and then lyophilized to give I-724 (35.31 mg, 36.28 μmol, 28.12% yield, 100% purity) as a white solid. LCMS (Method D): Rt: 0.434 min, [M+H]⁺=973.7. ¹H NMR (400 MHz, METHANOL-d4) δ=8.42-8.26 (m, 1H), 8.01-7.74 (m, 3H), 7.63-6.99 (m, 6H), 5.01-4.91 (m, 1H), 4.71-4.58 (m, 1H), 4.52-4.31 (m, 3H), 4.13-3.34 (m, 11H), 3.29-3.08 (m, 5H), 2.85-2.06 (m, 5H), 2.05-1.41 (m, 18H), 1.36-1.05 (m, 6H), 0.96-0.72 (m, 4H). SFC: Rt=1.536 min, 1.960 min. ¹⁹F NMR (377 MHz, METHANOL-d4) δ=−120.501, −122.108.

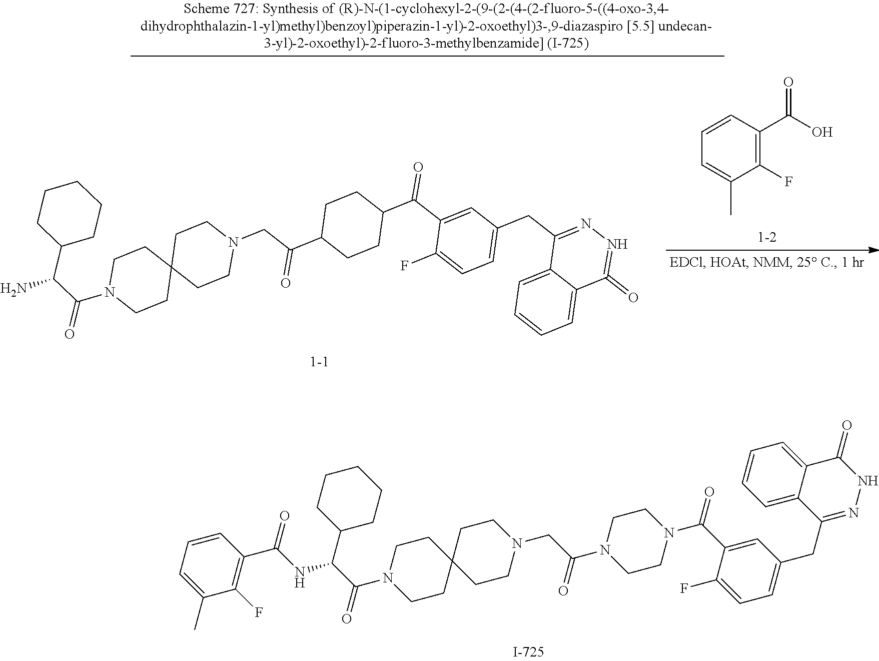

To a solution of Intermediate 1-1 (100 mg, 135.81 μmol, 1 eq, HCl salt) and Intermediate 1-2 (25.12 mg, 162.97 μmol, 1.2 eq) in DMF (1 mL) was added EDCI (52.07 mg, 271.62 μmol, 2 eq) and HOAt (18.49 mg, 135.81 μmol, 19.00 μL, 1 eq) and NMM (68.68 mg, 679.06 μmol, 74.66 μL, 5 eq). The mixture was stirred at 25° C. for 1 hr. To the reaction mixture was added H₂O (2 mL) and then it was extracted with DCM (2 mL*3), and the combined organic phase was washed, dried by Na₂SO₄, filtered and concentrated. The residue was purified by prep-HPLC (column: CD01-Phenomenex luna C18 150*25*10 um; mobile phase: [water (FA)-ACN]; gradient: 18%-48% B over 8 min) the eluent was concentrated to remove ACN and lyophilized to give product I-725 (39.12 mg, 43.40 μmol, 31.96% yield, 97.859% purity, FA salt) as a white solid. LCMS (Method D): Retention time: 0.433 min, [M+H]⁺=836.5. SFC: Retention time: 1.739 min. ¹H NMR (400 MHz, METHANOL-d4) δ=8.32 (d, J=2.0 Hz, 1H), 7.84 (d, J=5.2 Hz, 1H), 7.77 (d, J=7.6 Hz, 1H), 7.63-7.56 (m, 1H), 7.54-7.47 (m, 1H), 7.37 (d, J=3.6 Hz, 4H), 7.31 (t, J=3.2 Hz, 1H), 6.84 (t, J=3.6 Hz, 1H), 5.06-4.98 (m, 1H), 4.94 (d, J=8.4 Hz, 1H), 4.71-4.56 (m, 3H), 4.56-4.48 (m, 1H), 4.38-4.29 (m, 1H), 4.21-4.06 (m, 2H), 4.06-3.96 (m, 3H), 3.92 (s, 1H), 3.87-3.69 (m, 4H), 3.66-3.49 (m, 2H), 3.42-3.35 (m, 1H), 3.28-3.16 (m, 3H), 3.15-2.95 (m, 2H), 2.81 (s, 1H), 2.69-2.49 (m, 2H), 2.40-2.07 (m, 4H), 2.07-1.92 (m, 4H), 1.92-1.83 (m, 3H), 1.83-1.60 (m, 6H), 1.59-1.48 (m, 1H), 1.34-1.21 (m, 3H), 1.17-1.04 (m, 2H), 0.96-0.88 (m, 4H). ¹⁹F NMR (377 MHz, METHANOL-d₄), δ=−120.35 (br s, 1F), −120.67-−120.81 (m, 1F).

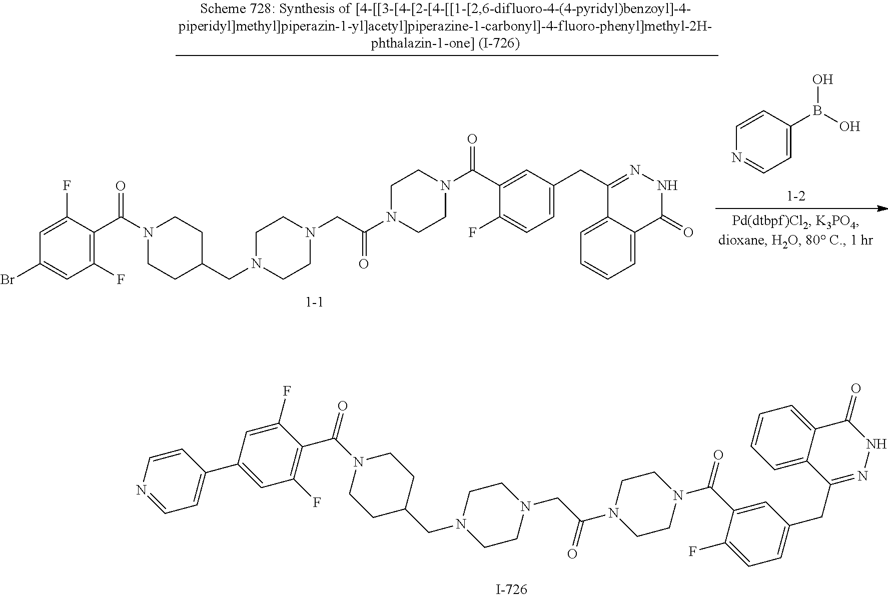 [SMILES: O=C(CN1CCN(CC2CCN(C(=O)c3c(F)cc(Br)cc3F)CC2)CC1)N1CCN(C(=O)c2cc(Cc3n[nH]c(=O)c4ccccc34)ccc2F)CC1 ; OB(O)c1ccncc1 ; O=C(CN1CCN(CC2CCN(C(=O)c3c(F)cc(-c4ccncc4)cc3F)CC2)CC1)N1CCN(C(=O)c2cc(Cc3n[nH]c(=O)c4ccccc34)ccc2F)CC1]

To a mixture of intermediate 1-1 (500 mg, 618.29 μmol, 1 eq) in dioxane (5 mL) and H₂O (1 mL) was added intermediate 1-2 (91.20 mg, 741.95 μmol, 1.2 eq), ditert-butyl(cyclopenta-1,4-dien-1-yl)phosphane; dichloropalladium; iron (40.30 mg, 61.83 μmol, 0.1 eq) and K₃PO₄ (393.72 mg, 1.85 mmol, 3 eq), and the mixture was degassed and purged with N₂ 3 times, and then the mixture was stirred at 80° C. for 1 h under N₂ atmosphere. The reaction mixture was diluted with H₂O (5 mL) and extracted with DCM (5 mL×3), the combined organic layers were dried over Na₂SO₄, filtered and concentrated to give a residue. The residue was purified by prep-HPLC (column: Phenomenex luna C18 150*40 mm*15 um; mobile phase: [water (FA)-ACN]; gradient: 0%-25% B over 10 min) and lyophilized to give the product. I-726 (207.53 mg, 225.36 μmol, 36.45% yield, 100.00% purity, TFA salt) was obtained as a white solid. LCMS (Method D): Retention time: 0.260 min, [M+H]⁺=807.5. ¹H NMR (400 MHz, METHANOL-d4) δ=8.73-8.62 (m, 2H), 8.41-8.36 (m, 1H), 8.00-7.93 (m, 1H), 7.93-7.82 (m, 2H), 7.81-7.75 (m, 2H), 7.63-7.57 (m, 2H), 7.51 (s, 1H), 7.43-7.34 (m, 1H), 7.18 (t, J=8.8 Hz, 1H), 4.72 (d, J=13.2 Hz, 1H), 4.40 (s, 2H), 3.86-3.73 (m, 2H), 3.70 (s, 2H), 3.63 (d, J=13.2 Hz, 1H), 3.57-3.50 (m, 2H), 3.45 (s, 1H), 3.37 (s, 2H), 3.31-3.14 (m, 2H), 2.96 (t, J=12.4 Hz, 1H), 2.85-2.58 (m, 8H), 2.52-2.39 (m, 2H), 1.97 (d, J=8.0 Hz, 2H), 1.84 (d, J=12.8 Hz, 1H), 1.34-1.14 (m, 2H). ¹⁹F NMR (377 MHz, METHANOL-d4) δ=−76.91 (s, 1F), −114.25 (br d, J=19.8 Hz, 2F), −120.75 (br d, J=33.9 Hz, 1F).

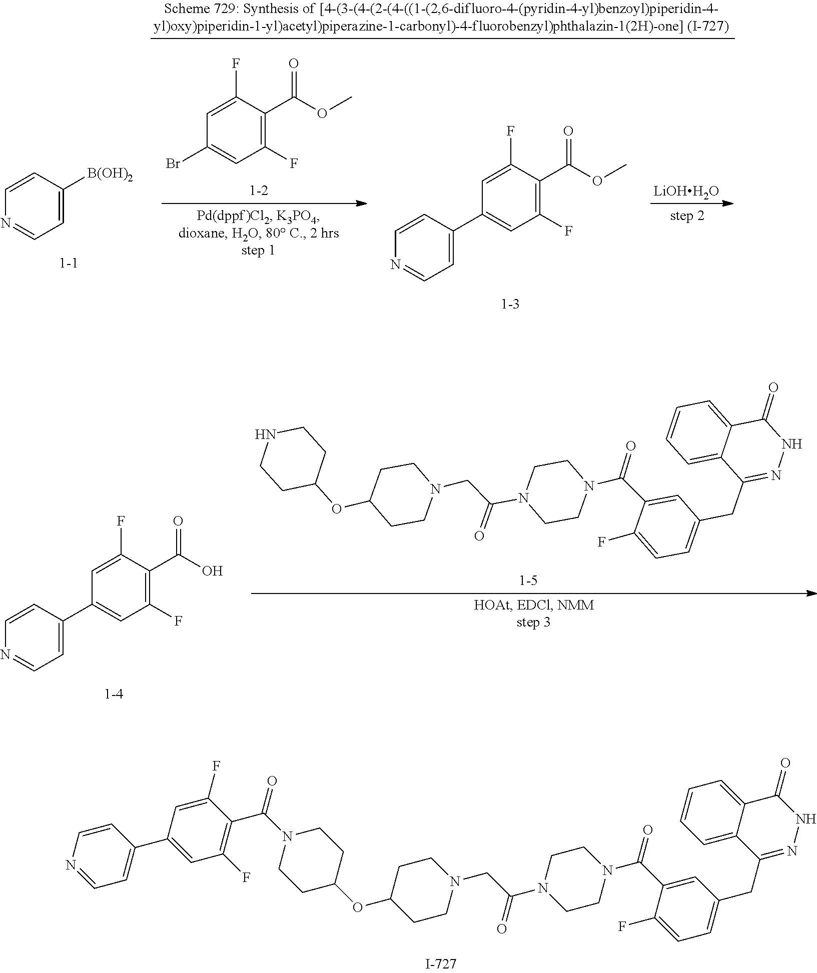

### Step 1: Synthesis of Intermediate 1-3.

A mixture of Intermediate 1-2 (2 g, 7.97 mmol, 1 eq), Intermediate 1-1 (1.08 g, 8.76 mmol, 1.1 eq), K₃PO₄ (5.07 g, 23.90 mmol, 3 eq), and Pd(dppf)Cl₂ (291.49 mg, 398.37 μmol, 0.05 eq) in dioxane (20 mL) and H₂O (2 mL) was degassed and purged with N₂ 3 times, and then the mixture was stirred at 80° C. for 2 hr under N₂ atmosphere. The reaction mixture was diluted with H₂O (20 mL) and extracted with EA (15 mL*3). The combined organic layers were dried over Na₂SO₄, filtered and concentrated under reduced pressure to give a residue. The residue was purified by flash silica gel chromatography (ISCO; 80 g SepaFlash Silica Flash Column, Eluent of 0˜40% Ethyl acetate/Petroleum ether gradient @60 mL/min), and the eluent was concentrated under reduced pressure to give a residue. Intermediate 1-3 (1.5 g, 5.99 mmol, 75.18% yield, 99.517% purity) was obtained as a pale yellow solid. LCMS (Method D): Retention time: 0.232 min, [M+H]⁺=250.1. ¹H NMR (400 MHz, METHANOL-d4) δ=8.70-8.66 (m, 2H), 7.82-7.76 (m, 2H), 7.58 (d, J=9.2 Hz, 2H), 3.98 (s, 3H). ¹⁹FNMR (376 MHz, METHANOL-d4) δ=−111.435.

### Step 2: Synthesis of Intermediate 1-4.

To a solution of Intermediate 1-3 (800 mg, 3.21 mmol, 1 eq) in THF (2 mL), MeOH (2 mL) H₂O (2 mL) was added LiOH·H₂O (404.12 mg, 9.63 mmol, 3 eq). The mixture was stirred at 25° C. for 1 hr. The mixture was adjusted to pH=5 with 1M HCl and filtered to give a residue. The crude product was used in the next step without further purification. Intermediate 1-4 (600 mg, 2.55 mmol, 79.47% yield) was obtained as a yellow solid. ¹H NMR (400 MHz, DMSO-d6) δ=8.67-8.59 (m, 2H), 7.81-7.69 (m, 2H), 7.49-7.37 (m, 2H). ¹⁹FNMR (376 MHz, METHANOL-d4) δ=−113.963.

### Step 3: Synthesis of I-727.

To a solution of Intermediate 1-4 (200 mg, 850.39 μmol, 1 eq) and Intermediate 1-5 (533.32 mg, 850.39 μmol, 1 eq, HCl salt) in DMF (3 mL) was added HOAt (115.75 mg, 850.39 μmol, 118.96 μL, 1 eq), EDCI (489.06 mg, 2.55 mmol, 3 eq) and NMM (430.07 mg, 4.25 mmol, 467.47 μL, 5 eq). The mixture was stirred at 25° C. for 1 hr. The reaction mixture was diluted with H₂O (10 mL) and extracted with EA (15 mL*3). The combined organic layers were washed with brine (15 mL*3), dried over Na₂SO₄, filtered and concentrated under reduced pressure to give a residue. The crude product was purified by reversed-phase chromatography (neutral condition), and the eluent was concentrated and lyophilized to give the desired product. I-727 (185.64 mg, 228.55 μmol, 26.88% yield, 99.46% purity) was obtained as a white solid. LCMS (Method D): Retention time: 0.285 min, (M+H)=808.1. ¹H NMR (400 MHz, METHANOL-d4) δ=8.70-8.61 (m, 2H), 8.40-8.32 (m, 1H), 7.98-7.81 (m, 3H), 7.77 (d, J=6.0 Hz, 2H), 7.62-7.54 (m, 2H), 7.52-7.34 (m, 2H), 7.20-7.10 (m, 1H), 4.38 (s, 2H), 4.09-4.01 (m, 1H), 3.83-3.47 (m, 11H), 3.36-3.32 (m, 1H), 3.27-3.16 (m, 3H), 2.77 (s, 2H), 2.34-2.18 (m, 2H), 1.98-1.80 (m, 4H), 1.70-1.51 (m, 4H). ¹⁹FNMR (376 MHz, METHANOL-d4) δ=−114.219, −120.749.

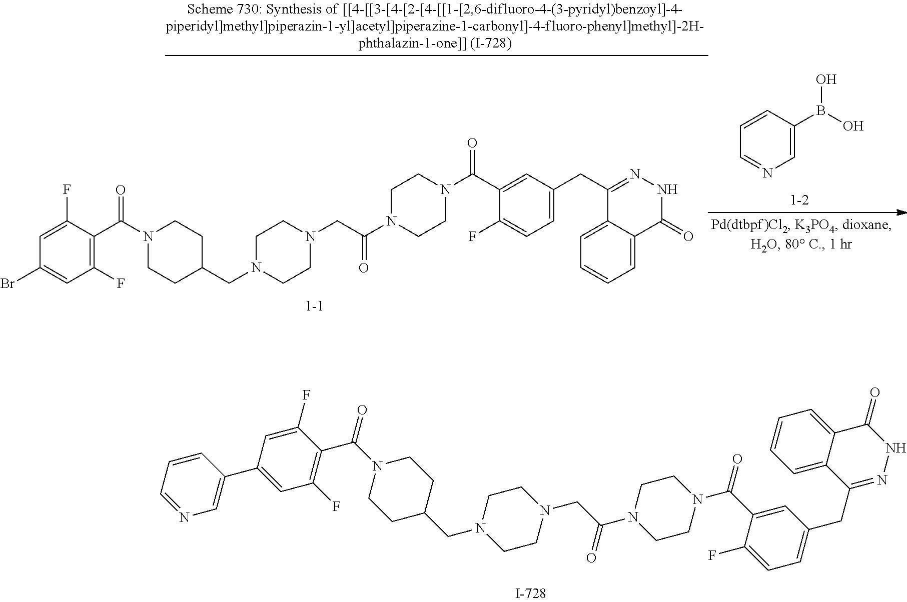 [SMILES: O=C(CN1CCN(CC2CCN(C(=O)c3c(F)cc(Br)cc3F)CC2)CC1)N1CCN(C(=O)c2cc(Cc3n[nH]c(=O)c4ccccc34)ccc2F)CC1 ; OB(O)c1cccnc1 ; O=C(CN1CCN(CC2CCN(C(=O)c3c(F)cc(-c4cccnc4)cc3F)CC2)CC1)N1CCN(C(=O)c2cc(Cc3n[nH]c(=O)c4ccccc34)ccc2F)CC1]

To a solution of Intermediate 1-2 (76.00 mg, 618.29 μmol, 1 eq) in dioxane (5 mL) and H₂O (1 mL) was added Intermediate 1-1 (500 mg, 618.29 μmol, 1 eq), K₃PO₄ (393.72 mg, 1.85 mmol, 3 eq), and ditert-butyl (cyclopenta-1,4-dien-1-yl)phosphane;dichloropalladium;iron (40.30 mg, 61.83 μmol, 0.1 eq). The mixture was stirred at 80° C. for 1 hr under N₂. The mixture was filtered. The filtrate was diluted with H₂O (5 mL) and extracted with EA (5 mL*2). The combined organic layers were dried over Na₂SO₄, filtered and concentrated under reduced pressure to give a residue. The residue was purified by reversed phase chromatography (0.1% FA). The eluent was concentrated under reduced pressure to remove ACN and then lyophilized to give the crude. The residue was purified by prep-HPLC (column: CD02-Waters Xbidge BEH C18 150*25*10 um; mobile phase: [water(NH₄HCO₃)-ACN]; gradient: 16%-46% B over 15 min). The eluent was concentrated under reduced pressure to remove ACN and then lyophilized to give I-728 (189.87 mg, 234.37 μmol, 37.91% yield, 99.6% purity) as a white solid. LCMS (Method D): Retention time: 0.289 min, [M+H]⁺=807.3. ¹H NMR (400 MHz, METHANOL-d4) δ=8.90 (d, J=1.6 Hz, 1H), 8.67-8.59 (m, 1H), 8.39 (d, J=7.6 Hz, 1H), 8.19 (d, J=8.0 Hz, 1H), 8.01-7.94 (m, 1H), 7.93-7.82 (m, 2H), 7.63-7.56 (m, 1H), 7.55-7.46 (m, 3H), 7.45-7.35 (m, 1H), 7.25-7.10 (m, 1H), 4.78-4.64 (m, 1H), 4.41 (s, 2H), 3.86-3.48 (m, 7H), 3.39-3.35 (m, 1H), 3.32-3.15 (m, 5H), 3.01-2.88 (m, 1H), 2.54 (s, 7H), 2.28-2.25 (m, 2H), 2.02-1.79 (m, 3H), 1.31-1.09 (m, 2H). ¹⁹F NMR (376 MHz, METHANOL-d4) δ=−114.415, −120.734.

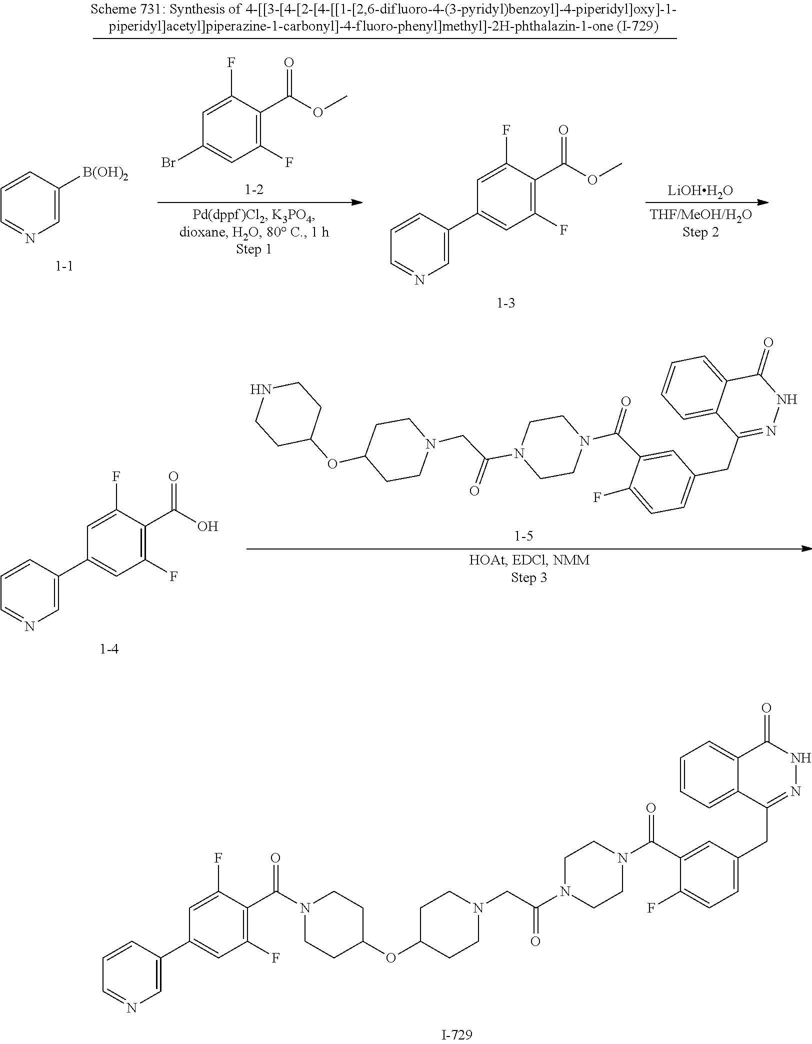

### Step 1: Synthesis of Intermediate 1-3.

To a mixture of Intermediate 1-1 (1 g, 8.14 mmol, 1 eq) and Intermediate 1-2 (2.04 g, 8.14 mmol, 1 eq) in dioxane (15 mL) and H₂O (3 mL) was added Pd(dppf)Cl₂ (595.28 mg, 813.55 μmol, 0.1 eq) and K₃PO₄ (5.18 g, 24.41 mmol, 3 eq), then the mixture was stirred at 80° C. for 1 hour under N₂. The reaction mixture was concentrated under reduced pressure to give a residue. The residue was purified by flash silica gel chromatography (ISCO; 40 g SepaFlash Silica Flash Column, Eluent of 0˜60% Ethyl acetate/Petroleum ether gradient @80 mL/min). The eluent was concentrated under reduced pressure to give Intermediate 1-3 (1.5 g, 6.02 mmol, 73.98% yield) as a white solid. LCMS (Method D): Retention time: 0.262 min, [M+H]⁺=249.8. ¹H NMR (400 MHz, CHLOROFORM-d), δ=8.85 (s, 1H), 8.69 (s, 1H), 7.88 (d, J=7.6 Hz, 1H), 7.44 (s, 1H), 7.23-7.18 (m, 2H), 3.99 (s, 3H). ¹⁹F NMR (376 MHz, METHANOL-d4), δ=−108.597.

### Step 2: Synthesis of Intermediate 1-4.

To a mixture of Intermediate 1-3 (1 g, 4.01 mmol, 1 eq) in THF (14 mL), MeOH (7 mL) and H₂O (3.5 mL) was added LiOH·H₂O (505.15 mg, 12.04 mmol, 3 eq), then the mixture was stirred at 20° C. for 1 hour. The reaction mixture was concentrated under reduced pressure to remove THF, and 1 N HCl was added (about 5 mL) to adjust the pH to 3-4 and the mixture was extracted with EA (10 mL*3). The combined organic layers were concentrated under reduced pressure to give Intermediate 1-4 (0.7 g, 2.98 mmol, 74.17% yield) as a white solid.

### Step 6: Synthesis of I-729.

To a mixture of Intermediate 1-4 (300 mg, 1.28 mmol, 1 eq) and Intermediate 1-5 (753.47 mg, 1.20 mmol, 9.42e−1 eq, HCl salt) in DMF (5 mL) was added HOAt (173.62 mg, 1.28 mmol, 178.44 μL, 1 eq), EDCI (733.59 mg, 3.83 mmol, 3 eq) and NMM (645.11 mg, 6.38 mmol, 701.20 μL, 5 eq), then the mixture was stirred at 20° C. for 0.5 hour. The reaction mixture was quenched with water (50 mL) and extracted with EA (10 mL*3). The combined organic layers were concentrated under reduced pressure to give a residue. The crude product was purified by reversed-phase chromatography (0.1% FA condition) twice. The eluent was concentrated under reduced pressure to remove MeCN and then lyophilized to give I-729 (221.84 mg, 259.07 μmol, 20.31% yield, 99.72% purity, FA salt) as a white solid. LCMS (Method D): Retention time: 0.298 min, [M+H]⁺=808.6. ¹H NMR (400 MHz, METHANOL-d4) δ=8.88 (d, J=2.0 Hz, 1H), 8.66-8.59 (m, 1H), 8.51-8.40 (m, 1H), 8.40-8.34 (m, 1H), 8.20-8.14 (m, 1H), 7.99-7.92 (m, 1H), 7.91-7.80 (m, 2H), 7.59-7.56 (m, 1H), 7.54-7.46 (m, 3H), 7.39-7.34 (m, 1H), 7.19-7.14 (m, 1H), 4.39 (s, 2H), 4.12-4.02 (m, 1H), 3.85-3.52 (m, 11H), 3.45 (s, 1H), 3.39-3.32 (m, 2H), 3.19-3.10 (m, 2H), 2.98-2.71 (m, 2H), 1.99 (d, J=2.8 Hz, 3H), 1.85 (s, 3H), 1.72-1.55 (m, 2H). ¹⁹F NMR (376 MHz, METHANOL-d4), δ=−114.490, δ=−120.704.

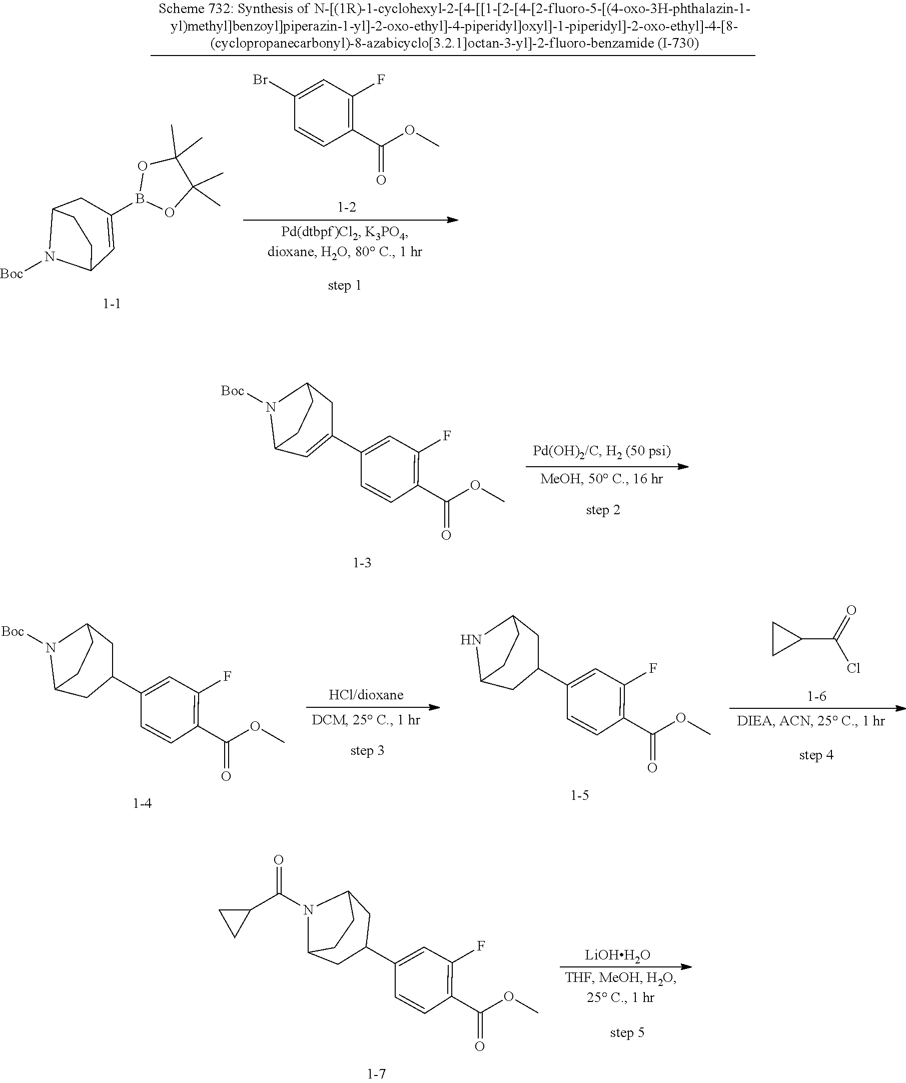

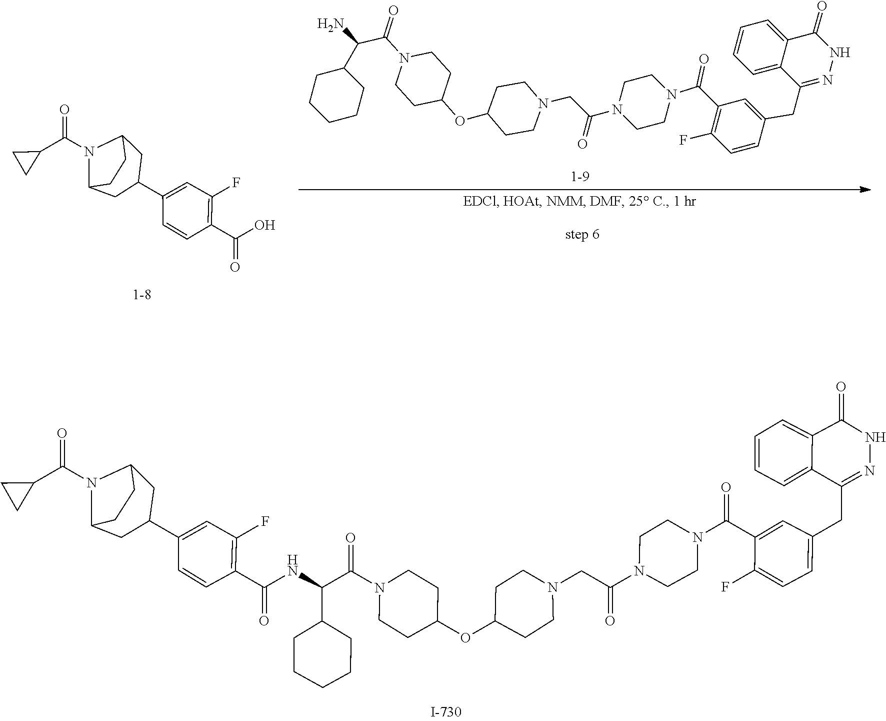 [SMILES: O=C(O)c1ccc(C2CC3CCC(C2)N3C(=O)C2CC2)cc1F ; N[C@@H](C(=O)N1CCC(OC2CCN(CC(=O)N3CCN(C(=O)c4cc(Cc5n[nH]c(=O)c6ccccc56)ccc4F)CC3)CC2)CC1)C1CCCCC1 ; O=C(N[C@@H](C(=O)N1CCC(OC2CCN(CC(=O)N3CCN(C(=O)c4cc(Cc5n[nH]c(=O)c6ccccc56)ccc4F)CC3)CC2)CC1)C1CCCCC1)c1ccc(C2CC3CCC(C2)N3C(=O)C2CC2)cc1F]

### Step 1: Synthesis of Intermediate 1-3

To a solution of Intermediate 1-1 (2.16 g, 6.44 mmol, 1.5 eq) and Intermediate 1-2 (1 g, 4.29 mmol, 1 eq) in H₂O (2 mL) and dioxane (10 mL) was added K₃PO₄ (2.73 g, 12.87 mmol, 3 eq) and ditert-butyl(cyclopenta-1,4-dien-1-yl)phosphane;dichloropalladium;iron (279.68 mg, 429.12 μmol, 0.1 eq). The mixture was stirred at 80° C. for 1 hr. Then the mixture was extracted with DCM (10 mL*3). The combined organic layers were dried over Na₂SO₄, filtered and concentrated under reduced pressure to give a residue. The residue was purified by flash silica gel chromatography (ISCO; 40 g SepaFlash Silica Flash Column, Eluent of 0˜23% Ethyl acetate/Petroleum ethergradient @60 mL/min) and concentrated under reduced pressure to give a residue. Intermediate 1-3 (2.097 g, crude) was obtained as a white solid. LCMS (Method D): Retention time: 0.483 min, [M-Boc+H]⁺=261.9.

### Step 2: Synthesis of Intermediate 1-4

To a solution Intermediate 1-3 (2.097 g, 5.80 mmol, 1 eq) in MeOH (20 mL) was added Pd(OH)₂/C (20%, 1.05 g) under N₂ atmosphere. The suspension was degassed and purged with H₂ 3 times. The mixture was stirred under H₂ (50 Psi) at 50° C. for 16 hr. The reaction mixture was filtered and concentrated under reduced pressure to give a residue. The crude product was used in the next step without further purification. Intermediate 1-4 (1.69 g, 4.65 mmol, 80.14% yield) was obtained as a yellow oil. LCMS (Method D): Retention time: 0.499 min, [M-tBu+H]⁺=308.2. ¹H NMR (400 MHz, METHANOL-d4) δ=7.89-7.81 (m, 1H), 7.23-7.03 (m, 2H), 4.31-4.23 (m, 2H), 3.91-3.86 (m, 3H), 2.51-2.37 (m, 1H), 2.09-1.96 (m, 2H), 1.93-1.85 (m, 2H), 1.78 (d, J=6.0 Hz, 2H), 1.72-1.59 (m, 2H), 1.50 (s, 9H). ¹⁹F NMR (377 MHz, METHANOL-d4) δ=−111.47.

### Step 3: Synthesis of Intermediate 1-5

To a solution of Intermediate 1-4 (1.39 g, 3.82 mmol, 1 eq) in DCM (7 mL) was added HCl/dioxane (2 M, 13.90 mL, 7.27 eq). The mixture was stirred at 25° C. for 1 hr. The reaction mixture was filtered and concentrated under reduced pressure to give a residue. The crude product was used in the next step without further purification. Intermediate 1-5 (1.128 g, crude, HCl salt) was obtained as a light yellow oil. LCMS (Method D): Retention time: 0.243 min, [M+H]⁺=263.9.

### Step 4: Synthesis of Intermediate 1-7

To a solution of Intermediate 1-5 (1.128 g, 3.76 mmol, 1 eq, HCl salt) in ACN (11 mL) was added DIEA (972.66 mg, 7.53 mmol, 1.31 mL, 2 eq). To the mixture was added Intermediate 1-6 (393.36 mg, 3.76 mmol, 341.45 μL, 1 eq) at 0° C. and it was stirred for 10 min. Then the mixture was stirred at 25° C. for 1 hr. The residue was purified by flash silica gel chromatography (ISCO; 40 g SepaFlash Silica Flash Column, Eluent of 0˜75% Ethyl acetate/Petroleum ether gradient @60 mL/min) and concentrated under reduced pressure to give a residue. Intermediate 1-7 (1.189 g, 3.59 mmol, 95.35% yield) was obtained as a yellow oil. LCMS (Method D): Retention time: 0.397 min, [M+H]⁺=332.1.

### Step 5: Synthesis of Intermediate 1-8

To a solution of Intermediate 1-7 (989 mg, 2.98 mmol, 1 eq) in THF (10 mL), MeOH (10 mL) and H₂O (5 mL) was added LiOH·H₂O (626.20 mg, 14.92 mmol, 5 eq). The mixture was stirred at 25° C. for 1 hr. The reaction mixture was concentrated under reduced pressure and adjusted to pH=2 with citric acid (1 M). Then the mixture was extracted with CHCl₃: Isopropyl alcohol=3:1 (15 mL*3). The combined organic layers were dried over Na₂SO₄, filtered and concentrated under reduced pressure to give a residue. The crude product was used in the next step without further purification. Intermediate 1-8 (1.3 g, crude) was obtained as a white solid. LCMS (Method D): Retention time: 0.355 min, [M+H]⁺=318.0. SFC: Retention time=1.632 min, 1.743 min.

### Step 6: Synthesis of I-730

To a solution of Intermediate 1-8 (18.22 mg, 57.42 μmol, 1.1 eq) and Intermediate 1-9 (40 mg, 52.20 μmol, 1 eq, HCl salt) in DMF (0.4 mL) was added EDCI (30.02 mg, 156.59 μmol, 3 eq), NMM (26.40 mg, 260.98 μmol, 28.69 μL, 5 eq) and HOAt (7.10 mg, 52.20 μmol, 7.30 μL, 1 eq). The mixture was stirred at 25° C. for 1 hr. The reaction mixture was filtered and concentrated under reduced pressure to give a residue. The residue was purified by flash silica gel chromatography (ISCO; 4 g SepaFlash Silica Flash Column, Eluent of 0˜66% Ethyl acetate/MeOH @20 mL/min) and concentrated under reduced pressure to give a residue. The crude product was purified by reversed phase chromatography (0.1% FA condition) followed by lyophilization to give product. I-730 (23.58 mg, 21.88 μmol, 41.92% yield, 99.782% purity, FA salt) was obtained as a white solid. LCMS (Method D): Retention time: 0.422 min, [M+H]⁺=1029.4. SFC: Retention time: 1.241 min, 1.686 min. ¹H NMR (400 MHz, METHANOL-d₄) δ=8.51 (s, 1H), 8.37 (d, J=7.6 Hz, 1H), 8.00-7.92 (m, 1H), 7.91-7.80 (m, 2H), 7.70 (t, J=7.6 Hz, 1H), 7.49 (d, J=2.4 Hz, 1H), 7.38 (s, 1H), 7.30-7.06 (m, 3H), 4.97 (d, J=7.2 Hz, 1H), 4.73-4.60 (m, 2H), 4.39 (s, 2H), 4.07-3.95 (m, 1H), 3.94-3.64 (m, 6H), 3.63-3.34 (m, 7H), 3.30-3.11 (m, 3H), 2.95-2.76 (m, 2H), 2.75-2.49 (m, 1H), 2.49-2.32 (m, 2H), 2.24-2.10 (m, 1H), 2.10-1.88 (m, 7H), 1.88-1.76 (m, 7H), 1.75-1.46 (m, 7H), 1.35-1.05 (m, 5H), 0.99-0.75 (m, 4H). ¹⁹F NMR (377 MHz, METHANOL-d₄), δ=−115.21-−115.56, −120.61-−120.89.

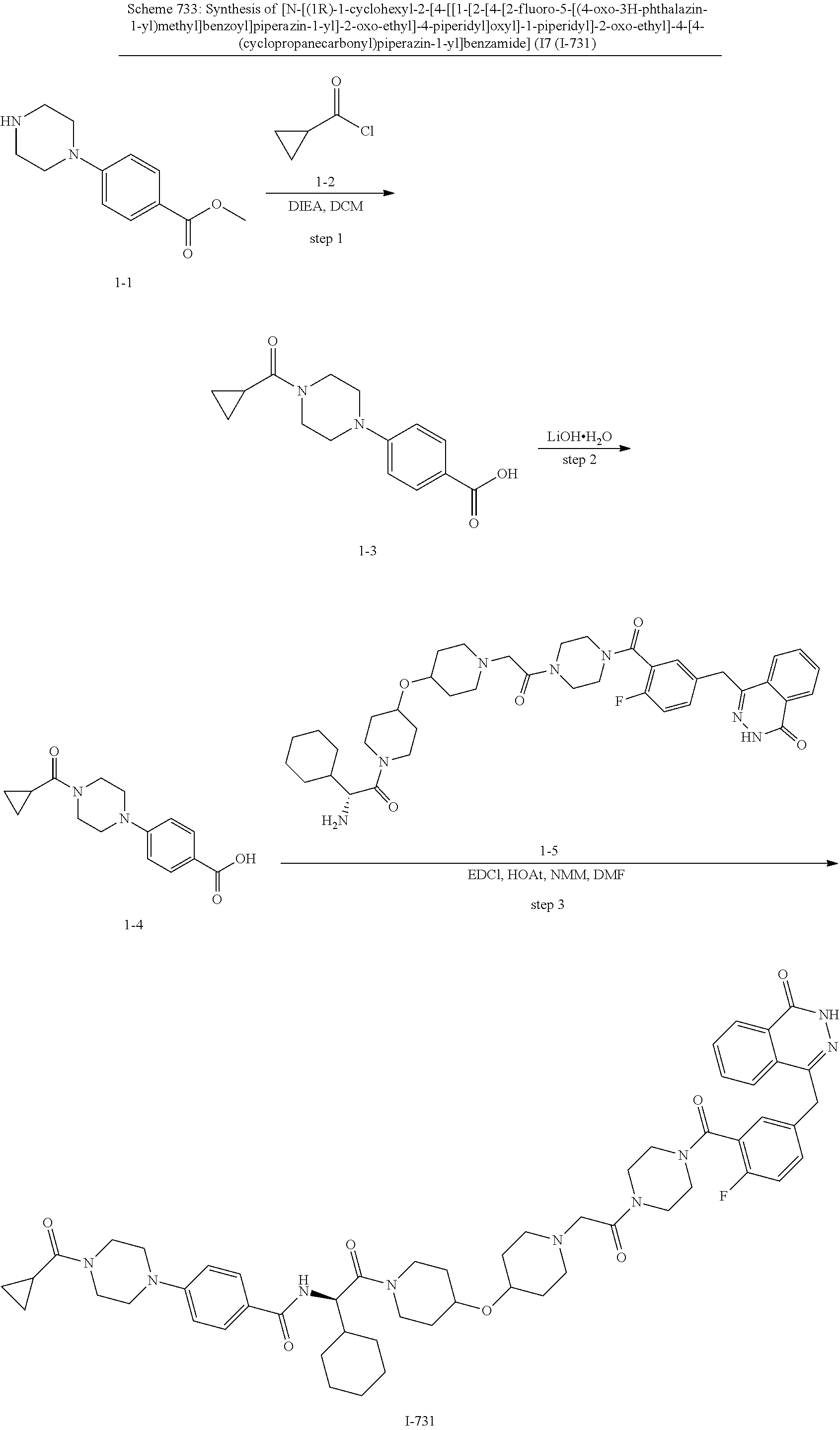

### Step 1: Synthesis of Intermediate 1-3

To a solution of Intermediate 1-1 (0.2 g, 907.99 μmol, 1 eq) in DCM (2 mL) was added DIEA (352.05 mg, 2.72 mmol, 474.46 μL, 3 eq), then Intermediate 1-2 (113.90 mg, 1.09 mmol, 98.87 μL, 1.2 eq) was added at 0° C. The mixture was stirred at 0° C. for 1 hr. The reaction mixture was quenched by H₂O (2 mL), and then extracted with DCM (3 mL*2). The combined organic layers were washed with NaCl (aq) (3 mL), dried over Na₂SO₄, filtered and concentrated under reduced pressure to give a residue. The residue was purified by flash silica gel chromatography (ISCO; 12 g SepaFlash Silica Flash Column, Eluent of 0˜100% Ethyl acetate/Petroleum ether gradient @40 mL/min) and the eluent was concentrated under reduced pressure to give Intermediate 1-3 (70 mg, 242.77 μmol, 26.74% yield) as a white solid. LCMS (Method H): Retention time: 0.574 min, [M+H]⁺=289.1. ¹H NMR (400 MHz, METHANOL-d4) δ=7.92-7.86 (m, 2H), 7.01-6.95 (m, 2H), 3.93 (s, 2H), 3.85 (s, 3H), 3.75 (s, 2H), 3.50-3.33 (m, 4H), 2.04-1.97 (m, 1H), 0.94-0.89 (m, 2H), 0.88-0.82 (m, 2H).

### Step 2: Synthesis of Intermediate 1-4

To a solution of Intermediate 1-3 (60 mg, 208.09 μmol, 1 eq) in MeOH (0.2 mL), THF (0.2 mL), and H₂O (0.2 mL) was added LiOH·H₂O (26.20 mg, 624.26 μmol, 3 eq). The mixture was stirred at 25° C. for 12 hr. The reaction mixture was adjusted to pH=6 by HCl (1M), and then extracted with DCM (5 mL*2). The combined organic layers were washed with NaCl(aq) (5 mL), dried over Na₂SO₄, filtered and concentrated under reduced pressure to give Intermediate 1-4 (60 mg, crude) as a white solid. LCMS (Method H): Retention time: 0.277 min, [M+H]⁺=275.1.

### Step 3: Synthesis of I-731

To a solution of Intermediate 1-4 (55 mg, 200.50 μmol, 1 eq) and Intermediate 1-5 (153.65 mg, 200.50 μmol, 1 eq, HCl salt) in DMF (2 mL) was added EDCI (115.31 mg, 601.50 μmol, 3 eq), HOAt (27.29 mg, 200.50 μmol, 28.05 μL, 1 eq), and NMM (101.40 mg, 1.00 mmol, 110.22 μL, 5 eq). The mixture was stirred at 25° C. for 0.5 hr. The reaction mixture was filtered and concentrated under reduced pressure to give a residue. The residue was purified by prep-HPLC (column: CD01-Phenomenex luna C18 150*25*10 um; mobile phase: [water(FA)-ACN]; gradient: 19%-49% B over 10 min) and the eluent was concentrated to remove ACN and lyophilized to give I-731 (108.28 mg, 104.30 μmol, 52.02% yield, 99.429% purity, FA salt) as a white solid. LCMS (Method D): Retention time: 0.400 min, [M+H]⁺=986.5. SFC: Retention time: 2.097 min. ¹H NMR (400 MHz, METHANOL-d4) δ=8.52 (s, 1H), 8.39 (d, J=7.6 Hz, 1H), 7.96 (d, J=7.2 Hz, 1H), 7.92-7.82 (m, 2H), 7.79 (d, J=8.8 Hz, 2H), 7.51 (d, J=3.6 Hz, 1H), 7.39 (s, 1H), 7.21-7.16 (m, 1H), 7.06-6.98 (m, 2H), 4.97-4.91 (m, 2H), 4.40 (s, 2H), 4.10-4.01 (m, 1H), 3.95 (s, 3H), 3.84-3.74 (m, 6H), 3.73-3.58 (m, 4H), 3.57-3.48 (m, 4H), 3.43 (s, 3H), 3.02-2.87 (m, 2H), 2.65-2.43 (m, 2H), 2.09-1.97 (m, 2H), 1.97-1.83 (m, 6H), 1.78 (s, 3H), 1.73-1.56 (m, 5H), 1.56-1.45 (m, 1H), 1.35-1.20 (m, 3H), 1.16-1.04 (m, 2H), 0.95-0.90 (m, 2H), 0.89-0.83 (m, 2H). ¹⁹F NMR (376 MHz, METHANOL-d4) δ=−117.92-−122.13 (m, 1F).

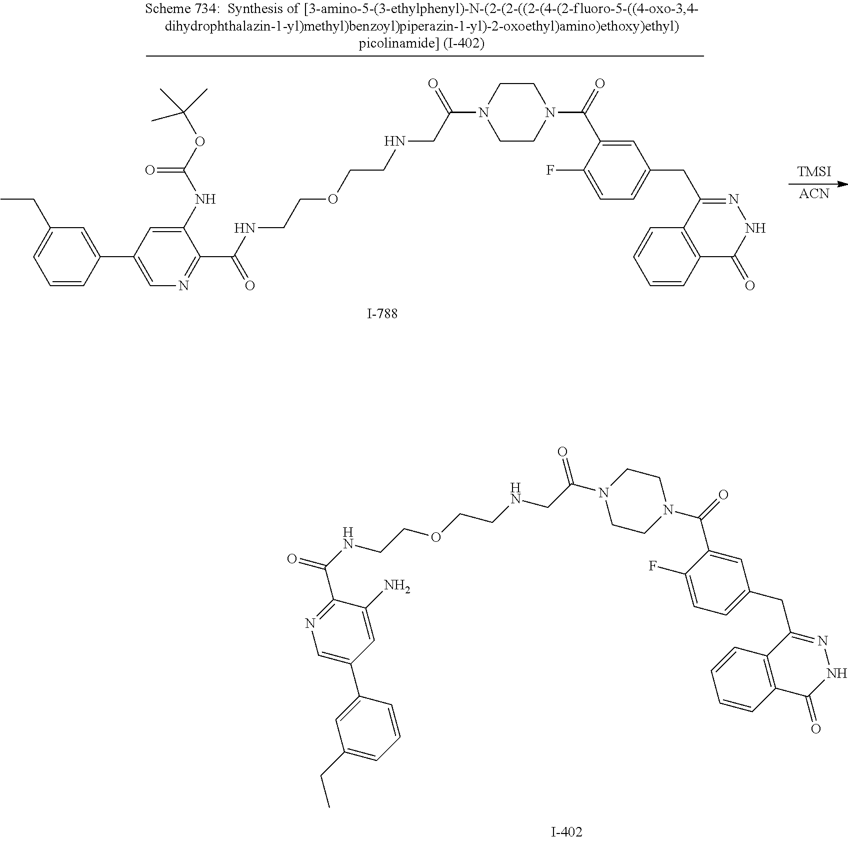

A solution of I-788 (40 mg, 45.41 μmol, 1 eq, FA salt) and TMSI (27.26 mg, 136.22 μmol, 18.54 L, 3 eq) in ACN (0.5 mL) was stirred at 25 C for 0.3 hr. The reaction mixture was concentrated under reduced pressure to give a residue. The residue was purified by prep-HPLC (column: Phenomenex luna C18 150*25 mm*10 um; mobile phase: [water(FA)-ACN]; gradient: 17%-47% B over 10 min) and the eluent was concentrated to remove ACN and lyophilized to give I-402 (10.48 mg, 13.38 μmol, 29.46% yield, 99.672% purity, FA salt) as a white solid. LCMS (Method D): Retention time: 0.384 min, (M+H)=735.5. ¹H NMR (400 MHz, METHANOL-d₄) δ=8.58-8.51 (m, 1H), 8.40-8.30 (m, 1H), 8.12-7.99 (m, 1H), 7.97-7.90 (m, 1H), 7.89-7.80 (m, 2H), 7.50-7.31 (m, 6H), 7.28-7.22 (m, 1H), 7.18-7.07 (m, 1H), 4.39-4.30 (m, 2H), 3.78-3.57 (m, 12H), 3.48 (s, 2H), 3.25 (s, 2H), 2.99 (s, 2H), 2.75-2.65 (m, 2H), 1.29-1.22 (m, 3H). ¹⁹F NMR (376 MHz, METHANOL-d₄) δ=−120.6.

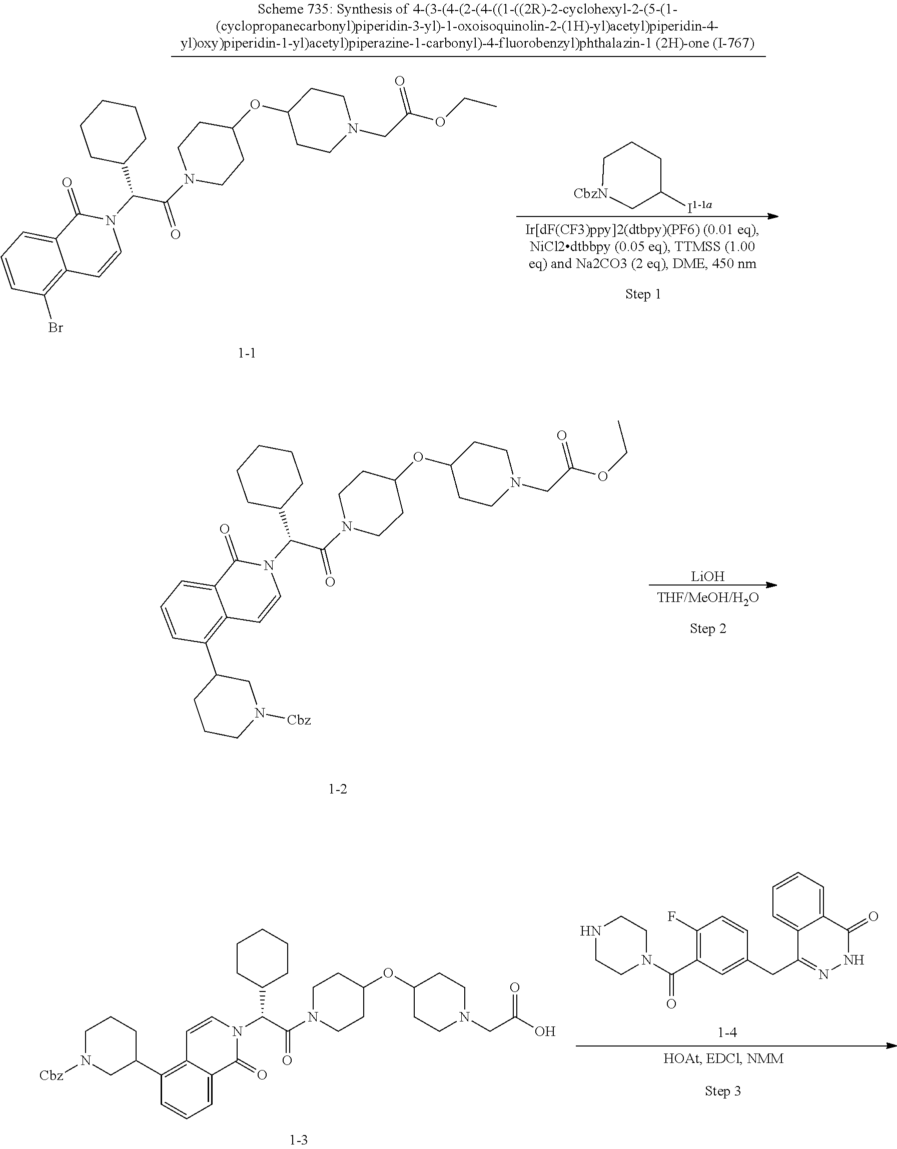

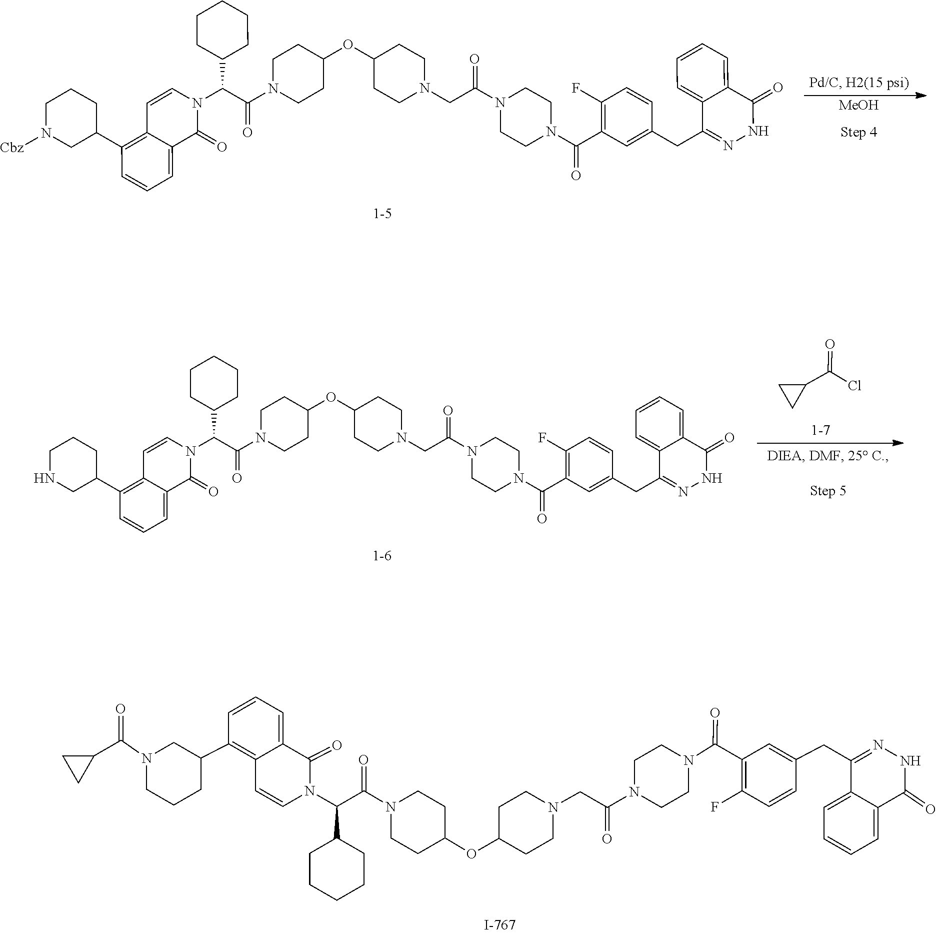 [SMILES: "MeOH" ; O=C(CN1CCC(OC2CCN(C(=O)[C@@H](C3CCCCC3)n3ccc4c(C5CCCN(C(=O)OCc6ccccc6)C5)cccc4c3=O)CC2)CC1)N1CCN(C(=O)c2cc(Cc3n[nH]c(=O)c4ccccc34)ccc2F)CC1 ; "Step 4" ; O=C(CN1CCC(OC2CCN(C(=O)[C@@H](C3CCCCC3)n3ccc4c(C5CCCNC5)cccc4c3=O)CC2)CC1)N1CCN(C(=O)c2cc(Cc3n[nH]c(=O)c4ccccc34)ccc2F)CC1 ; O=C(Cl)C1CC1 ; O=C(CN1CCC(OC2CCN(C(=O)[C@@H](C3CCCCC3)n3ccc4c(C5CCCN(C(=O)C6CC6)C5)cccc4c3=O)CC2)CC1)N1CCN(C(=O)c2cc(Cc3n[nH]c(=O)c4ccccc34)ccc2F)CC1]

### Step 1: Synthesis of Intermediate 1-2

A mixture of Intermediate 1-1 (1 g, 1.62 mmol, 1 eq), Intermediate 1-1a (727.76 mg, 2.11 mmol, 1.3 eq), TTMSS (403.29 mg, 1.62 mmol, 500.35 μL, 1 eq), Ir[dF(CF₃)ppy]₂(dtbpy)(PF₆) (18.20 mg, 16.22 μmol, 0.01 eq), Na₂CO₃ (515.70 mg, 4.87 mmol, 3 eq) and 4-tert-butyl-2-(4-tert-butyl-2-pyridyl)pyridine; dichloronickel (3.23 mg, 8.11 μmol, 0.005 eq) in DME (20 mL) was degassed and purged with N₂, and then the mixture was stirred at 25° C. for 16 hr and irradiated with a 455 nm blue LED. The mixture was diluted with water (10 mL) and extracted with EtOAc (15 mL*3). The organic layer was concentrated under vacuum. The residue was purified by Pre-HPLC (column: Waters Xbridge Prep OBD C18 150*40 mm*10 um; mobile phase: [water(NH₄HCO₃)-ACN]; gradient: 588%-88% B over 15 min) and concentrated under vacuum. Intermediate 1-2 (600 mg, 794.75 μmol, 49.00% yield) was obtained as a yellow solid. LCMS (Method G): Rt=0.854, 0.874 min, [M+H]+=755.4.

### Step 2: Synthesis of Intermediate 1-3

To a solution of Intermediate 1-2 (450 mg, 596.06 μmol, 1 eq) in THF (2 mL), H₂O (1 mL) and MeOH (1 mL) was added LiOH·H₂O (50.02 mg, 1.19 mmol, 2 eq). The mixture was stirred at 25° C. for 1 hr. The mixture was concentrated under vacuum and adjusted to pH=5 with 2N HCl and extracted with EtOAc (10 mL*3). The organic layer was dried with anhydrous Na₂SO₄, filtered and concentrated under vacuum. Intermediate 1-3 (430 mg, 591.55 umol, 99.24% yield) was obtained as a yellow solid. LCMS (Method G): Rt=0.567, 0.590 min, [M+H]+=727.4.

### Step 3: Synthesis of Intermediate 1-5

To a mixture of Intermediate 1-3 (430 mg, 591.55 μmol, 1 eq), Intermediate 1-4 (216.74 mg, 591.55 μmol, 1 eq) and HOAt (80.52 mg, 591.55 μmol, 82.75 μL, 1 eq) in DMF (5 mL) was added EDCI (226.80 mg, 1.18 mmol, 2 eq) and NMM (299.18 mg, 2.96 mmol, 325.19 μL, 5 eq). The mixture was stirred at 25° C. for 2 hrs. The mixture was poured into water (5 mL) and filtered. The filter cake was concentrated under vacuum. The residue was purified by Pre-HPLC (column: Waters Xbridge Prep OBD C18 150*40 mm*10 um; mobile phase: [water(NH₄HCO₃)-ACN]; gradient: 50%-80% B over 15 min) and concentrated under vacuum. Intermediate 1-5 (530 mg, 492.90 μmol, 83.32% yield) was obtained as a yellow solid. LCMS (Method): Rt=2.329, 2.395 min, [M+H]+=1075.9.

### Step 4: Synthesis of Intermediate 1-6

To a solution of Pd(OH)₂/C (100.00 mg, 142.41 μmol, 20% purity, 7.66e−1 eq) in DMF (2 mL) was added Intermediate 1-5 (0.2 g, 186.00 μmol, 1 eq). The suspension was degassed and purged with H₂ 3 times. The mixture was stirred under H₂ (50 Psi) at 60° C. for 12 hr. The mixture was filtered through a pad of Celite and washed with DMF (2 mL*3). Intermediate 1-6 (175 mg, crude) was obtained as brown liquid in DMF. LCMS (Method G): Rt=0.764, 0.824 min, [M+H]+=941.7.

### Step 5: Synthesis of I-767

To the solution of Intermediate 1-6 (175 mg, 185.94 μmol, 1 eq) in DMF (8 mL) was added Intermediate 1-7 (38.88 mg, 371.89 μmol, 33.75 μL, 2 eq) and DIEA (72.09 mg, 557.83 μmol, 97.16 μL, 3 eq). The mixture was diluted with water (1 mL), extracted with EtOAc (3 mL*2) and washed with brine (1 mL*3). The organic layer was concentrated under vacuum. The residue was purified by Pre-HPLC (column: Waters Xbridge 150*25 mm*5 um; mobile phase: [water(NH₄HCO₃)-ACN]; gradient: 40%-70% B over 10 min) and dried by lyophilization. I-767 (19.64 mg, 19.13 μmol, 10.29% yield, 98.325% purity) was obtained as a white solid. LCMS (Method G): Rt=0.697, 0.723 min. SFC: Rt=2.709, 4.089 min ¹H NMR (400 MHz, METHANOL-d4) δ=8.41-8.32 (m, 1H), 8.31-8.23 (m, 1H), 7.94 (br d, J=7.6 Hz, 1H), 7.90-7.79 (m, 2H), 7.78-7.66 (m, 1H), 7.57-7.44 (m, 3H), 7.37 (br s, 1H), 7.16-7.15 (m, 1H), 7.11-6.98 (m, 1H), 5.89-5.77 (m, 1H), 4.73-4.57 (m, 2H), 4.49-4.35 (m, 3H), 4.12-3.90 (m, 2H), 3.84-3.60 (m, 6H), 3.58-3.40 (m, 5H), 3.25-3.16 (m, 2H), 2.85-2.66 (m, 3H), 2.41-2.19 (m, 3H), 2.14-1.92 (m, 4H), 1.89-1.74 (m, 6H), 1.73-1.59 (m, 4H), 1.59-1.43 (m, 3H), 1.37-1.17 (m, 5H), 1.16-0.73 (m, 7H). ¹⁹F NMR (377 MHz, METHANOL-d4) δ=−120.80 (br s, 1F).

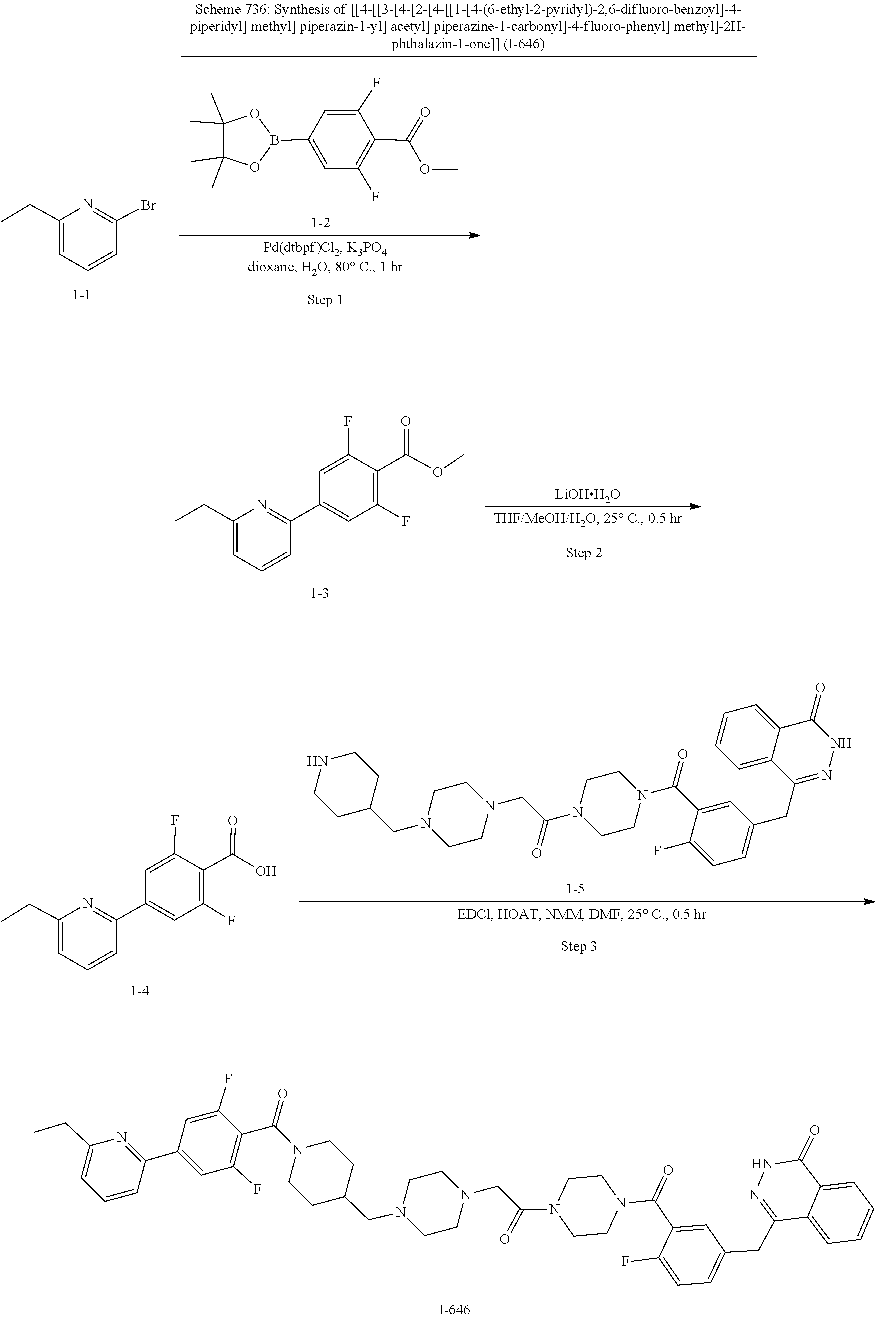

### Step 1: Synthesis of Intermediate 1-3

A mixture of intermediate 1-1 (100 mg, 537.49 μmol, 1 eq), intermediate 1-2 (160.22 mg, 537.49 μmol, 1 eq), Pd(dtbpf)Cl₂ (35.03 mg, 53.75 μmol, 0.1 eq), and K₃PO₄ (342.28 mg, 1.61 mmol, 3 eq) in dioxane (1 mL) and H₂O (0.2 mL) was degassed and purged with N₂ 3 times, and then the mixture was stirred at 80° C. for 1 hr under N₂ atmosphere. The reaction mixture was concentrated under reduced pressure to give a residue. The residue was purified by flash silica gel chromatography (ISCO; 12 g SepaFlashSilica Flash Column, Eluent of 0˜100% PE/EA@36 mL/min, PE/EA=10:1, Rf=0.5) and the eluent was concentrated under reduced pressure to give a product. Intermediate 1-3 (140 mg, crude) was obtained as a yellow gum. LCMS (Method D): Rt=0.363 min, [M+H]⁺=278.0.

### Step 2: Synthesis of Intermediate 1-4

To a solution of intermediate 1-3 (140 mg, 504.93 μmol, 1 eq) in MeOH (1 mL), THF (1 mL) and H₂O (1 mL) was added LiOH H₂O (63.57 mg, 1.51 mmol, 3 eq). The mixture was stirred at 25° C. for 0.5 hr. The reaction mixture was poured into H₂O (2 mL) at 25° C., the pH was adjusted to 7 with 1M aqueous HCl, and then the mixture was extracted with EA (2 mL*3). The combined organic layers were dried over Na₂SO₄, filtered and concentrated under reduced pressure to give a product. The product was used in the next step without purification. Intermediate 1-4 (90 mg, 308.53 μmol, 61.10% yield, 90.24% purity) was obtained as a yellow gum. LCMS (Method D): Rt=0.190 min, [M+H]⁺=264.1.

### Step 3: Synthesis of I-646

To a solution of intermediate 1-4 (30 mg, 113.96 μmol, 1 eq) and intermediate 1-5 (71.36 mg, 113.96 μmol, 1 eq, HCl salt) in DMF (0.7 mL) was added EDCI (65.54 mg, 341.89 μmol, 3 eq), HOAt (15.51 mg, 113.96 μmol, 15.94 μL, 1 eq) and NMM (57.64 mg, 569.82 μmol, 62.65 μL, 5 eq). The mixture was stirred at 25° C. for 0.5 hr. The reaction mixture was concentrated under reduced pressure to give a residue. The residue was purified by prep-HPLC (column: CD01-Phenomenex luna C18 150*25*10 um; mobile phase: [water (FA)-ACN]; gradient: 12%-42% B over 10 min) and the eluent was concentrated to remove MeCN and then lyophilized. I-646 (15.96 mg, 17.86 μmol, 15.68% yield, 98.61% purity, FA salt) was obtained as a white solid. LCMS (Method D): Rt=0.342 min, [M+H]⁺=835.2. ¹H NMR (400 MHz, METHANOL-d4) δ=8.45-8.40 (m, 1H), 8.40-8.33 (m, 1H), 7.98-7.91 (m, 1H), 7.90-7.85 (m, 1H), 7.85-7.76 (m, 4H), 7.76-7.72 (m, 1H), 7.49 (s, 1H), 7.41-7.33 (m, 1H), 7.30 (d, J=7.6 Hz, 1H), 7.17 (t, J=8.8 Hz, 1H), 4.70 (d, J=12.0 Hz, 1H), 4.39 (s, 2H), 3.82-3.72 (m, 2H), 3.71-3.60 (m, 3H), 3.55-3.48 (m, 2H), 3.42 (s, 1H), 3.35-3.33 (m, 2H), 3.26-3.11 (m, 2H), 3.00-2.80 (m, 4H), 2.70 (d, J=14.8 Hz, 7H), 2.49-2.39 (m, 2H), 2.02-1.90 (m, 2H), 1.82 (t, J=12.4 Hz, 1H), 1.35 (t, J=7.6 Hz, 3H), 1.27-1.15 (m, 2H). ¹⁹F NMR (377 MHz, METHANOL-d4) δ=−115.270, −120.779.

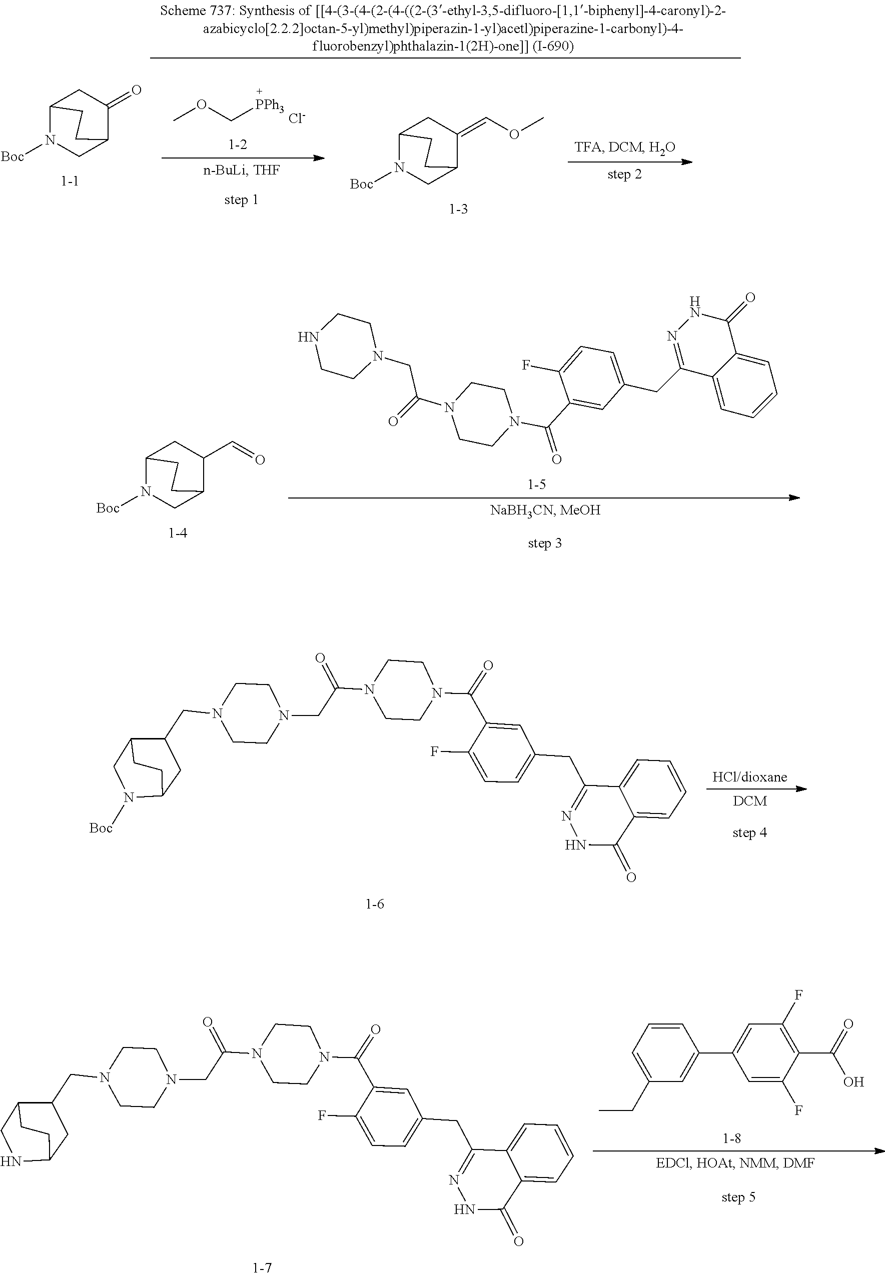

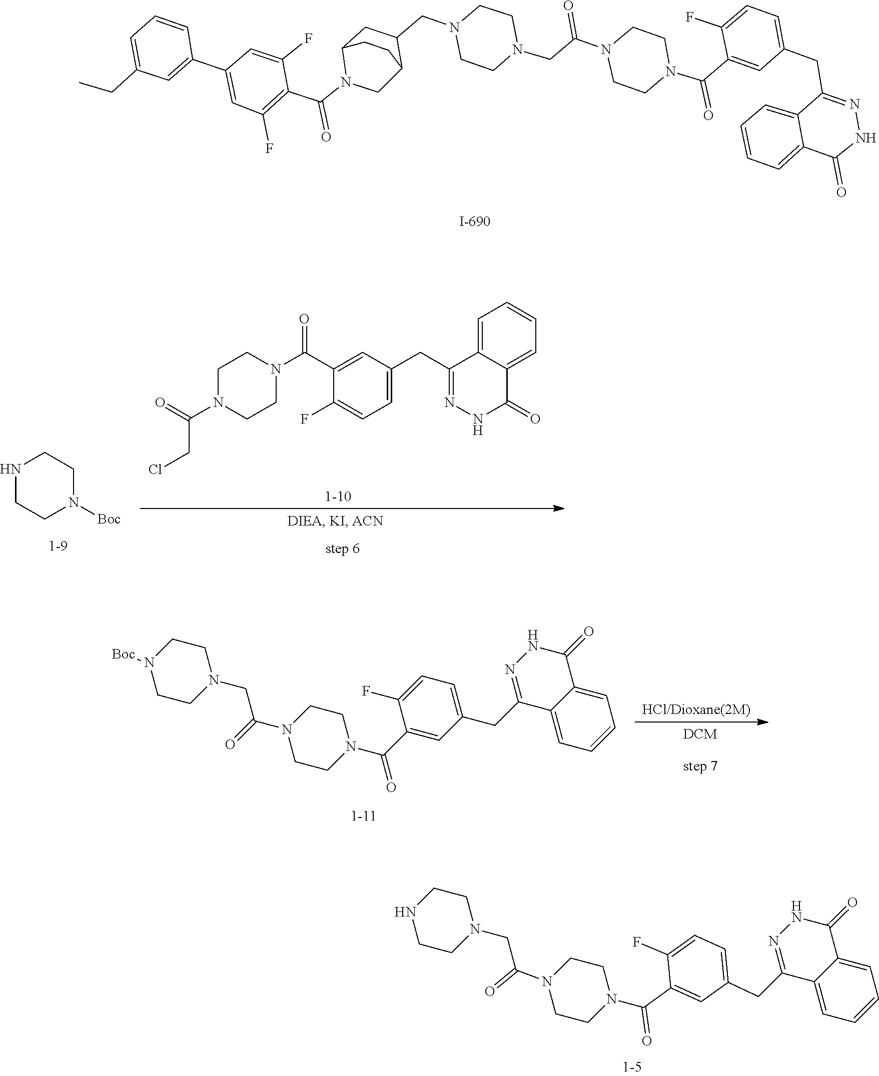 [SMILES: CCc1cccc(-c2cc(F)c(C(=O)N3CC4CCC3CC4CN3CCN(CC(=O)N4CCN(C(=O)c5cc(Cc6n[nH]c(=O)c7ccccc67)ccc5F)CC4)CC3)c(F)c2)c1 ; CC(C)(C)OC(=O)N1CCNCC1 ; O=C(CCl)N1CCN(C(=O)c2cc(Cc3n[nH]c(=O)c4ccccc34)ccc2F)CC1 ; CC(C)(C)OC(=O)N1CCN(CC(=O)N2CCN(C(=O)c3cc(Cc4n[nH]c(=O)c5ccccc45)ccc3F)CC2)CC1 ; O=C(CN1CCNCC1)N1CCN(C(=O)c2cc(Cc3n[nH]c(=O)c4ccccc34)ccc2F)CC1]

### Step 1: Synthesis of Intermediate 1-3

To a solution of Intermediate 1-2 (4.56 g, 13.32 mmol, 1.5 eq) in THF (10 mL) was added n-BuLi (2.5 M, 5.33 mL, 1.5 eq) at −10° C. under N₂ atmosphere. The reaction mixture was stirred at 0° C. for 1 h. A solution of Intermediate 1-1 (2 g, 8.88 mmol, 1 eq) in THF (10 mL) was added dropwise to the reaction solution at −20° C. The mixture was stirred for 2 h at 25° C. under N₂ atmosphere. The mixture was quenched with a sat. NH₄Cl solution (40 mL) under N₂ atmosphere and extracted with EA (20 mL*3). The combined organic layers were dried over Na₂SO₄, filtered and concentrated under reduced pressure to give the residue. The crude was purified by Flash Silica-gel chromatography (ISCO; 40 g SepaFlash Silica Flash Column, Eluent of 16-20% EA/PE) and the eluent was combined and concentrated to give the target product. Intermediate 1-3 (1.3 g, 5.13 mmol, 57.80% yield) was obtained as colorless oil. ¹H NMR (400 MHz, CHLOROFORM-d) δ=6.01-5.70 (m, 1H), 4.31-3.99 (m, 1H), 3.63-3.50 (m, 3H), 3.45-3.26 (m, 2H), 3.06-2.88 (m, 1H), 2.64-2.12 (m, 2H), 2.00-1.60 (m, 4H), 1.46 (s, 9H).

### Step 2: Synthesis of Intermediate 1-4

To a solution of Intermediate 1-3 (1.3 g, 5.13 mmol, 1 eq) in DCM (11.7 mL) was added H₂O (3.9 mL) and TFA (3.51 g, 30.79 mmol, 2.29 mL, 6 eq). The resulting mixture was stirred at 25° C. for 2 hrs. The reaction mixture was quenched by saturated NaHCO₃ (20 mL), and extracted with DCM (5 mL*3). The organic phase was combined and concentrated to afford the crude. The crude was purified by flash silica gel chromatography (ISCO; 12 g SepaFlash Silica Flash Column, Eluent of 15˜25% Petroleum ether/Ethyl acetate gradient @20 mL/min). The eluent was combined and concentrated to afford the target product. Intermediate 1-4 (0.4 g, 1.59 mmol, 30.94% yield, 95% purity) was obtained as a colorless oil. LCMS (Method E): Rt=0.512 min, [M+H-tBu]⁺=184.4.

### Step 3: Synthesis of Intermediate 1-6

To a solution of Intermediate 1-4 (0.4 g, 1.67 mmol, 1 eq) in MeOH (40 mL) was added Intermediate 1-5 (823.27 mg, 1.67 mmol, 1 eq, 2 HCl salt). The resulting mixture was stirred at 25° C. for 1.5 hrs. Then NaBH₃CN (210.08 mg, 3.34 mmol, 2 eq) was introduced into the system. The system was kept at 25° C. and stirred for another 16 hrs. The reaction mixture was quenched by addition of NH₄Cl aq. (30 mL). The mixture was then concentrated to remove MeOH (40 mL) then basified by addition of NaHCO₃. The suspension was extracted by DCM (50 mL*3, 25% V/V MeOH). The organic phase was combined and concentrated to afford the crude. The crude was purified by prep-HPLC (FA condition), and the eluent was combined and basified. The resulting suspension was extracted by DCM (30 mL*4, 25% V/V MeOH), and the organic layers were combined and concentrated to afford the target product. Intermediate 1-6 (0.9 g, 1.23 mmol, 73.71% yield, 98% purity) was obtained as an off-white solid. LCMS (Method E): Rt=0.444 min, [M+H]⁺=716.2. ¹H NMR (400 MHz, DMSO-d₆) δ=12.68-12.47 (m, 1H), 8.26 (d, J=7.6 Hz, 1H), 7.99-7.94 (m, 1H), 7.92-7.86 (m, 1H), 7.85-7.79 (m, 1H), 7.48-7.40 (m, 1H), 7.40-7.32 (m, 1H), 7.28-7.18 (m, 1H), 4.35-4.32 (m, 2H), 3.86-3.71 (m, 2H), 3.62 (d, J=7.2 Hz, 6H), 3.27-2.98 (m, 6H), 2.47-2.01 (m, 10H), 1.99-1.46 (m, 7H), 1.40-1.35 (m, 9H), 1.25-1.11 (m, 1H).

### Step 4: Synthesis of Intermediate 1-7

Intermediate 1-6 (1.5 g, 2.10 mmol, 1 eq) was suspended in HCl/dioxane (25 mL) and stirred at 25° C. for 1 hr. The reaction mixture was concentrated directly to afford the crude. The crude was used in the next step of the reaction without purification. Intermediate 1-7 (1.4 g, crude, 3HCl salt) was obtained as a white solid. LCMS (Method E): Rt=0.361 min, [M+H]⁺=616.2. ¹H NMR (400 MHz, DMSO-d₆) δ=12.70-12.46 (m, 1H), 8.30-8.23 (m, 1H), 8.01-7.78 (m, 3H), 7.52-7.43 (m, 1H), 7.42-7.32 (m, 1H), 7.30-7.20 (m, 1H), 5.63-4.88 (m, 3H), 4.73-4.30 (m, 4H), 3.95-3.63 (m, 8H), 3.50-2.89 (m, 12H), 2.42-1.27 (m, 9H).

### Step 5: Synthesis of I-690

To a solution of Intermediate 1-7 (1.4 g, 2.15 mmol, 1 eq, 3HCl salt) in DMF (30 mL) was added Intermediate 1-8 (562.94 mg, 2.15 mmol, 1 eq), NMM (1.74 g, 17.17 mmol, 1.89 mL, 8 eq), HOAt (292.17 mg, 2.15 mmol, 300.28 μL, 1 eq) and EDCI (1.23 g, 6.44 mmol, 3 eq). The resulting mixture was stirred at 25° C. for 1 hr. The reaction mixture was diluted by water (150 mL) and extracted by EA (30 mL*3), the organic phases were combined and concentrated to afford the crude. The crude was combined with another lot of material (100 mg) and purified by Prep-HPLC (FA condition). The eluent was combined and lyophilized to afford the target product. I-690 (0.73279 g, 809.50 μmol, 37.71% yield, 95% purity, FA salt) was obtained as a white solid. LCMS (Method E): Rt=1.229 min, 1.248 min, [M+H]⁺=860.7. SFC: Rt=2.332 min, 2.669 min, 3.240 min, 4.106 min ¹H NMR (400 MHz, DMSO-d₆) δ=12.60 (s, 1H), 8.31-8.22 (m, 1H), 8.16 (s, 1H), 8.00-7.93 (m, 1H), 7.92-7.86 (m, 1H), 7.85-7.79 (m, 1H), 7.67-7.51 (m, 4H), 7.48-7.33 (m, 3H), 7.32-7.18 (m, 2H), 4.47-4.25 (m, 2H), 3.78-3.04 (m, 13H), 2.72-2.63 (m, 2H), 2.49-1.48 (m, 17H), 1.33-1.05 (m, 4H). ¹⁹F NMR (377 MHz, DMSO-d₆) δ=−114.03-−114.97 (m, 2F), −119.77 (s, 1F).

### Step 6: Synthesis of Intermediate 1-11

To a solution of Intermediate 1-10 (3.0 g, 6.77 mmol, 1 eq) and Intermediate 1-9 (1.26 g, 6.77 mmol, 1 eq) in ACN (30 mL) was added DIEA (3.50 g, 27.10 mmol, 4.72 mL, 4 eq) and KI (112.45 mg, 677.40 μmol, 0.1 eq). The mixture was stirred at 70° C. for 1 hr. The mixture was filtered, and the filtrate was concentrated under reduced pressure to afford the crude. The crude was purified by Flash Silica-gel chromatography (ISCO; 40 g SepaFlash Silica Flash Column, Eluent of 14-18% EA/PE) and the eluent was concentrated to give target product. Intermediate 1-11 (2.2 g, 3.71 mmol, 54.80% yield) was obtained as a yellow oil. LCMS (Method E): Rt=0.421 min, [M+H]⁺=593.5.

### Step 7: Synthesis of Intermediate 1-5

Intermediate 1-11 (2.2 g, 2.00 mmol, 1 eq) was dissolved in HCl/dioxane (40 mL) (2M) and stirred for 2 hrs at 25° C. The suspension was concentrated to afford the crude product. After work-up, Intermediate 1-5 (1.8 g, crude, 2HCl salt) was obtained as a yellow oil. LCMS (Method G): Rt=0.491 min, [M+H]⁺=493.3.

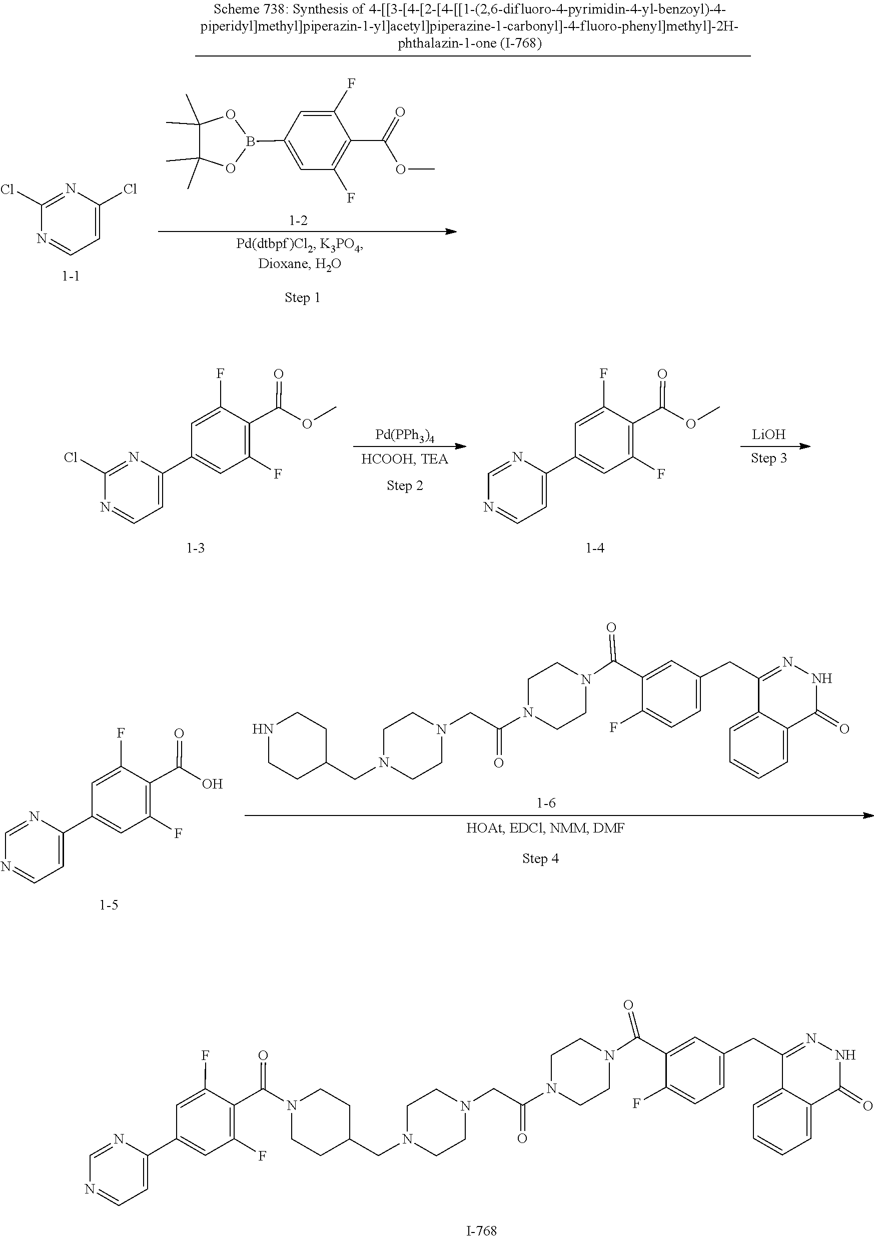

### Step 1: Synthesis of Intermediate 1-3.

To a mixture of Intermediate 1-1 (1.2 g, 8.05 mmol, 3 eq) and Intermediate 1-2 (800.36 mg, 2.68 mmol, 1 eq) in dioxane (15 mL) and H₂O (3 mL) was added ditert-butyl(cyclopenta-1,4-dien-1-yl)phosphane; dichloropalladium;iron (174.99 mg, 268.50 μmol, 0.1 eq) and K₃PO₄ (1.71 g, 8.05 mmol, 3 eq) and then the mixture was stirred at 60° C. for 2 hours under N₂. The reaction mixture was concentrated under reduced pressure to give a residue. The residue was purified by flash silica gel chromatography (ISCO; 40 g SepaFlash Silica Flash Column, Eluent of 0˜40% Ethyl acetate/Petroleum ether gradient @80 mL/min). The eluent was concentrated under reduced pressure to give Intermediate 1-3 (500 mg, 1.76 mmol, 65.42% yield) as a white solid. LCMS (Method D): Retention time: 0.398 min, [M+H]⁺=284.7.

### Step 2: Synthesis of Intermediate 1-4.

To a mixture of Intermediate 1-3 (400 mg, 1.41 mmol, 1 eq) in DMF (10 mL) was added Pd(PPh₃)₄ (162.39 mg, 140.53 μmol, 0.1 eq), HCOOH (135.02 mg, 2.81 mmol, 2 eq) and TEA (142.20 mg, 1.41 mmol, 195.59 μL, 1 eq), then the mixture was stirred at 110° C. for 1 hour under N₂. The reaction mixture was diluted with water (50 mL) and extracted with EA (10 mL*3). The combined organic layers were concentrated under reduced pressure to give a residue. The residue was purified by flash silica gel chromatography (ISCO; 40 g SepaFlash Silica Flash Column, Eluent of 0-30% Ethylacetate/Petroleum ether gradient @80 mL/min). The eluent was concentrated under reduced pressure to give Intermediate 1-4 (300 mg, 1.07 mmol, 75.94% yield, 89% purity) as a white solid. LCMS (Method D): Retention time: 0.347 min, [M+H]⁺=250.9. ¹H NMR (400 MHz, METHANOL-d4) δ=9.27 (d, J=1.1 Hz, 1H), 8.89 (d, J=5.4 Hz, 1H), 8.08 (dd, J=1.4, 5.4 Hz, 1H), 7.99-7.92 (m, 2H), 3.97 (s, 3H). ¹⁹F NMR (376 MHz, METHANOL-d4), δ=−111.570.

### Step 3: Synthesis of Intermediate 1-5.

To a mixture of Intermediate 1-4 (200 mg, 799.36 μmol, 1 eq) in MeOH (2 mL), H₂O (1 mL) and THF (4 mL) was added LiOH·H₂O (100.63 mg, 2.40 mmol, 3 eq), then the mixture was stirred at 20° C. for 0.5 hour. The reaction mixture was concentrated under reduced pressure to remove THF and MeOH, then 1 N HCl (about 2 mL) was added to adjust the pH to 1-2, and the mixture was filtered and concentrated under reduced pressure to give Intermediate 1-5 (150 mg, 635.12 μmol, 79.45% yield) as a white solid. LCMS (Method D): Retention time: 0.234 min, [M+H]⁺=236.8.

### Step 4: Synthesis of I-768.

To a mixture of Intermediate 1-5 (120 mg, 508.10 μmol, 1 eq) and Intermediate 1-6 (318.15 mg, 508.10 μmol, 1 eq, HCl salt) in DMF (2 mL) was added EDCI (292.21 mg, 1.52 mmol, 3 eq), NMM (256.96 mg, 2.54 mmol, 279.31 μL, 5 eq) and HOAt (69.16 mg, 508.10 μmol, 71.08 μL, 1 eq), then the mixture was stirred at 20° C. for 0.5 hour. The reaction mixture was diluted with water (50 mL) and extracted with EA (10 mL*3). The combined organic layers were concentrated under reduced pressure to give a residue. The crude product was purified by reversed-phase column chromatography (0.1% FA condition). The eluent was concentrated under reduced pressure to remove MeCN and then lyophilized to give I-768 (150 mg, 173.30 μmol, 34.11% yield, 98.65% purity, FA salt) as a white solid. LCMS (Method D): Retention time: 0.307 min, [M+H]⁺=808.6. ¹H NMR (400 MHz, METHANOL-d4) δ=9.26 (d, J=1.2 Hz, 1H), 8.89 (d, J=5.6 Hz, 1H), 8.41 (s, 1H), 8.39-8.34 (m, 1H), 8.07-8.04 (m, 1H), 8.02-7.92 (m, 3H), 7.91-7.79 (m, 2H), 7.50 (d, J=2.8 Hz, 1H), 7.41-7.33 (m, 1H), 7.17 (t, J=9.2 Hz, 1H), 4.70 (d, J=12.4 Hz, 1H), 4.39 (s, 2H), 3.87-3.47 (m, 8H), 3.40 (s, 2H), 3.29-3.18 (m, 2H), 2.94 (t, J=12.4 Hz, 1H), 2.82-2.54 (m, 8H), 2.47-2.34 (m, 2H), 2.02-1.91 (m, 2H), 1.87-1.82 (m, 1H), 1.28-1.14 (m, 2H). ¹⁹F NMR (376 MHz, METHANOL-d4), δ=−114.309, δ=−120.806.

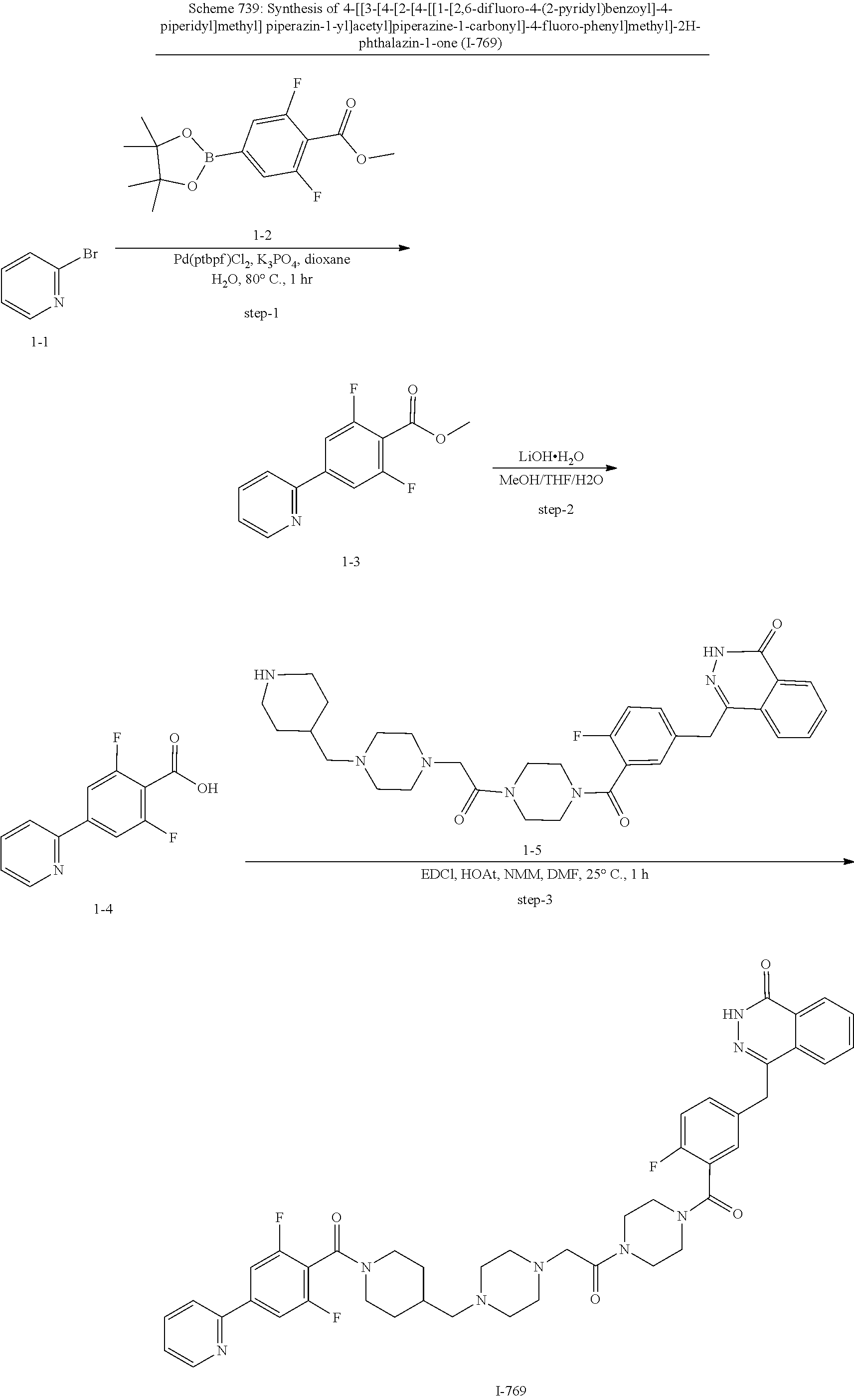

### Step 1: Synthesis of Intermediate 1-3

To a solution of Intermediate 1-1 (265.01 mg, 1.68 mmol, 159.94 μL, 1 eq) in dioxane (5 mL) was added methyl Intermediate 1-2 (0.5 g, 1.68 mmol, 1 eq), ditert-butyl(cyclopenta-1,4-dien-1-yl)phosphane;dichloropalladium;iron (109.32 mg, 167.73 μmol, 0.1 eq), K₃PO₄ (1.07 g, 5.03 mmol, 3 eq) and H₂O (1 mL). The resulting mixture was purged with nitrogen 3 times. Then the system was allowed to reach 80° C. and stirred for 1 hr. The reaction mixture was concentrated under reduced pressure. The residue was then diluted with water (10 mL) and extracted with EA (3 mL*3). The organic phase was combined and concentrated to afford the crude. The crude was purified by reverse-phase prep-HPLC (FA condition). The eluent was combined and concentrated to afford the target product. After purification, Intermediate 1-3 (0.366 g, 1.37 mmol, 81.43% yield, 93% purity) was obtained as a white solid. LCMS (Method E): Retention time=0.524 min, (M+H)⁺=507.2. ¹H NMR (400 MHz, CHLOROFORM-d) δ=8.86-8.61 (m, 1H), 7.88-7.78 (m, 1H), 7.76-7.70 (m, 1H), 7.69-7.61 (m, 2H), 7.39-7.30 (m, 1H), 3.98 (s, 3H).

### Step 2: Synthesis of Intermediate 1-4

To a solution of Intermediate 1-3 (0.366 g, 1.47 mmol, 1 eq) in MeOH (1.5 mL) was added THF (1.5 mL) and a solution of LiOH·H₂O (184.89 mg, 4.41 mmol, 3 eq) in H₂O (1.5 mL). The resulting mixture was stirred at 25° C. for 1 hr. The pH of the reaction mixture was adjusted to 6 via addition of 1M HCl. The suspension was then extracted by EA (3 mL*3). The organic phase was combined and concentrated to afford the crude. The crude was purified by reverse-phase prep-HPLC (FA condition). The eluent was combined and concentrated to afford the target product. After purification, Intermediate 1-4 (0.17 g, 722.83 μmol, 49.22% yield, 100% purity) was obtained as a white solid. LCMS (Method E): Retention time=0.364 min, (M+H)+=236.1. ¹H NMR: ¹H NMR (400 MHz, DMSO-d6) δ=14.20-13.87 (m, 1H), 8.83-8.57 (m, 1H), 8.15 (d, J=8.0 Hz, 1H), 8.04-7.87 (m, 3H), 7.56-7.38 (m, 1H).

### Step 3: Synthesis of I-769

To a solution of Intermediate 1-4 (0.16 g, 680.31 μmol, 1 eq) in DMF (6 mL) was added Intermediate 1-5 (583.91 mg, 680.31 μmol, 1 eq, HCl salt), EDCI (391.50 mg, 2.04 mmol, 3 eq), HOAt (92.66 mg, 680.31 μmol, 95.23 μL, 1 eq) and NMM (550.84 mg, 5.44 mmol, 598.74 μL, 8 eq) respectively. The resulting mixture was stirred at 25° C. for 1 hr. The reaction mixture was diluted by water (60 mL) then extracted by EA (20 mL*3). The organic phase was combined and concentrated under reduced pressure to afford the crude. The crude was purified by reverse-phase Prep-HPLC (FA condition.). The eluent was combined and lyophilized to yield the target product. I-769 (0.314 g, 387.45 μmol, 56.95% yield, 99.562% purity) was obtained as a white solid. LCMS (Method E): Retention time=0.427 min, (M+H)+=807.3. HPLC: Retention time=7.655 min. ¹H NMR (400 MHz, CHLOROFORM-d) δ=10.37-10.22 (m, 1H), 8.78-8.67 (m, 1H), 8.56-8.42 (m, 1H), 7.86-7.68 (m, 5H), 7.67-7.58 (m, 2H), 7.42-7.31 (m, 3H), 7.07 (s, 1H), 4.80 (d, J=13.6 Hz, 1H), 4.30 (s, 2H), 3.95-3.48 (m, 7H), 3.46-3.15 (m, 4H), 3.10 (t, J=12.8 Hz, 1H), 2.90-2.78 (m, 1H), 2.67-2.30 (m, 7H), 2.24 (s, 2H), 1.96-1.80 (m, 3H), 1.36-1.08 (m, 2H). ¹⁹F NMR (376 MHz, CHLOROFORM-d) δ=−112.59 (s, 2F), −117.36-−117.83 (m, 1F).

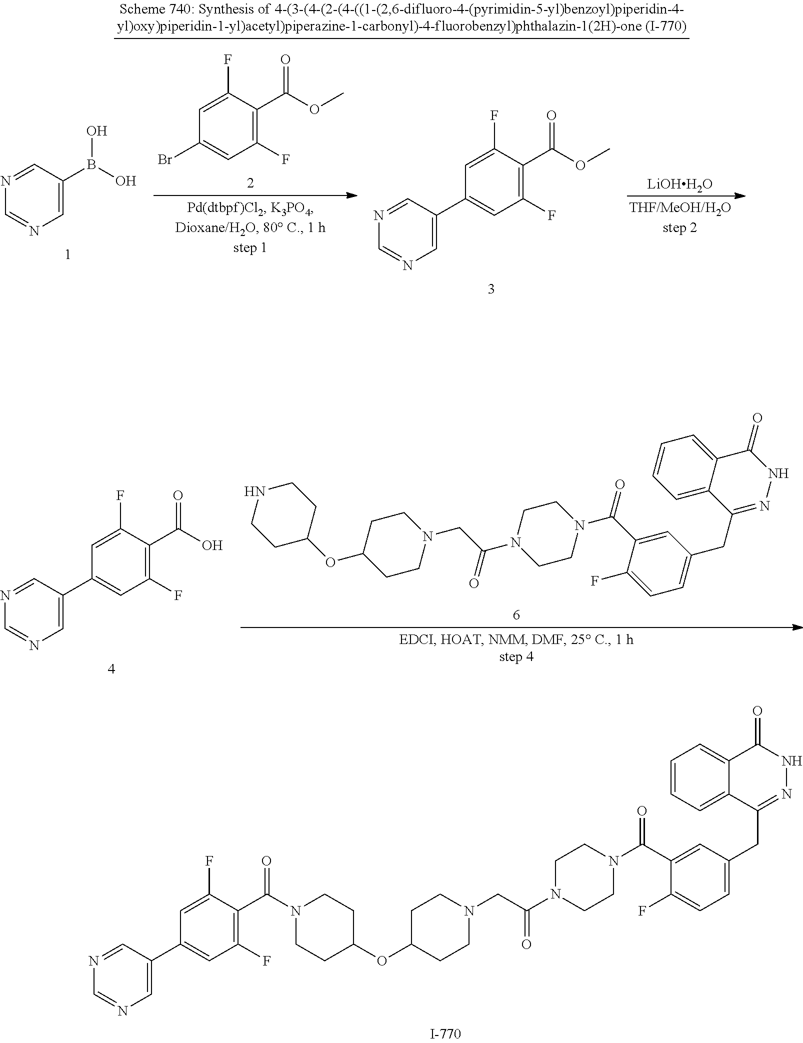

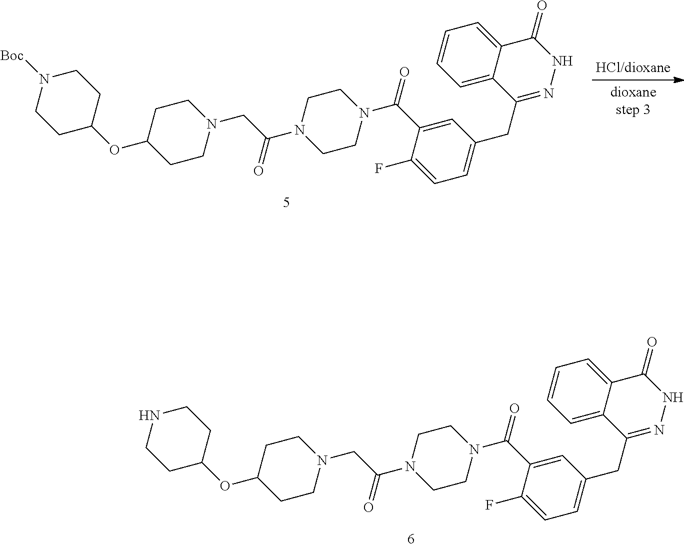 [SMILES: CC(C)(C)OC(=O)N1CCC(OC2CCN(CC(=O)N3CCN(C(=O)c4cc(Cc5n[nH]c(=O)c6ccccc56)ccc4F)CC3)CC2)CC1 ; O=C(CN1CCC(OC2CCNCC2)CC1)N1CCN(C(=O)c2cc(Cc3n[nH]c(=O)c4ccccc34)ccc2F)CC1]

### Step 1: Synthesis of Intermediate 3

To a solution of Intermediate 1 (700 mg, 5.65 mmol, 1 eq) and Intermediate 2 (1.42 g, 5.65 mmol, 1 eq) in dioxane (7.5 mL), H₂O (1.5 mL) was added Pd(dtbpf)Cl₂ (368.20 mg, 564.95 μmol, 0.1 eq) and K₃PO₄ (2.40 g, 11.30 mmol, 2 eq). The mixture was stirred at 80° C. for 1 hr. The mixture was diluted with water (12 mL) and extracted with DCM (12 mL*3). The combined organic layer was dried over Na₂SO₄, filtered and concentrated in vacuo to give a residue. The residue was purified by column chromatography (SiO₂, PE:EA=20:1 to PE:EA=0:1), then the organic liquid was concentrated in vacuo to give Intermediate 3 (500 mg, 1.74 mmol, 30.77% yield, 87% purity) as a yellow solid. LCMS (Method D): Rt=0.360 min, [M+H]⁺=251.0.

### Step 2: Synthesis of Intermediate 4

To a solution of Intermediate 3 (250 mg, 999.20 umol, 1 eq) in THF (1 mL), MeOH (1 mL), and H₂O (1 mL) was added LiOH·H₂O (125.79 mg, 3.00 mmol, 3 eq). The mixture was stirred at 25° C. for 0.5 hr. The mixture was concentrated and lyophilized to give a crude product (350 mg, crude). Then to the crude product was added H₂O (5 mL) and HCl (1 M, 3 mL, 2.08 eq). The mixture was stirred at 25° C. for 0.5 hr. The mixture was filtered and the filter cake was concentrated to give Intermediate 4 (220 mg, 809.39 μmol, 55.99% yield, 86.89% purity) as a gray solid. LCMS (Method E): Rt=0.353 min, [M+H]⁺=237.1.

### Step 3: Synthesis of Intermediate 6

To a solution of Intermediate 5 (5 g, 7.24 mmol, 1 eq) in dioxane (25 mL) was added HCl/dioxane (4 M, 25 mL, 13.82 eq). The mixture was stirred at 25° C. for 1 hr. The reaction mixture was concentrated to give Intermediate 6 (5 g, 6.94 mmol, 95.83% yield, 87% purity, HCl salt) as a yellow solid. LCMS (Method E): Rt=0.363 min, [M+H]⁺=591.2. ¹H NMR (400 MHz, DMSO-d6) δ=12.61 (br d, J=4.8 Hz, 1H), 9.74 (m, 1H), 9.29-8.91 (m, 2H), 8.26 (br d, J=8.0 Hz, 1H), 8.00-7.93 (m, 1H), 7.93-7.86 (m, 1H), 7.86-7.78 (m, 1H), 7.51-7.43 (m, 1H), 7.36 (m, 1H), 7.25 (m, 1H), 4.53-4.25 (m, 4H), 3.82-3.67 (m, 3H), 3.48-3.39 (m, 3H), 3.30 (br d, J=11.2 Hz, 3H), 3.23-3.00 (m, 5H), 2.98-2.84 (m, 2H), 2.14-2.01 (m, 1H), 2.00-1.82 (m, 4H), 1.79-1.61 (m, 3H). ¹⁹F NMR (400 MHz, DMSO-d6) δ=−119.71.

### Step 4: Synthesis of I-770

To a solution of Intermediate 4 (200 mg, 846.83 μmol, 1 eq) and Intermediate 6 (531.09 mg, 846.83 μmol, 1 eq, HCl salt) in DMF (2 mL) was added HOAt (172.89 mg, 1.27 mmol, 177.69 μL, 1.5 eq), EDCI (324.68 mg, 1.69 mmol, 2 eq) and NMM (428.27 mg, 4.23 mmol, 465.51 μL, 5 eq). The mixture was stirred at 25° C. for 1 hr. The reaction was quenched by water (1 mL). The mixture was purified by reversed phase HPLC (ISCO; 80 g SepaFlash C18 Column, Eluent of 0˜50% (0.1% FA) water/MeCN@60 mL/min). The eluent was concentrated to give a crude product. Then the crude product was purified by Prep-HPLC (column: Phenomenex luna C18 150*25 mm*10 um; mobile phase: [water(FA)-ACN]; gradient: 10%-40% B over 10 min). The eluent was concentrated and lyophilized to give I-770 (182 mg, 223.44 μmol, 26.39% yield, 99.3% purity) as a white solid. LCMS (Method E): Rt=0.416 min, [M+H]⁺=809.3. HPLC: Rt=7.307 min. ¹H NMR (400 MHz, DMSO-d6) δ=12.59 (s, 1H), 9.25 (d, J=1.6 Hz, 3H), 8.26 (d, J=7.6 Hz, 1H), 8.15 (s, 1H), 8.00-7.93 (m, 1H), 7.89 (m, 1H), 7.86-7.75 (m, 3H), 7.44 (br s, 1H), 7.37 (m, 1H), 7.23 (m, 1H), 4.33 (s, 2H), 4.09-3.92 (m, 1H), 3.80-3.68 (m, 1H), 3.67-3.55 (m, 3H), 3.54 (br s, 5H), 3.23-3.01 (m, 6H), 2.75-2.60 (m, 2H), 2.24-2.04 (m, 2H), 1.93-1.67 (m, 4H), 1.52-1.26 (m, 4H). ¹⁹F NMR (400 MHz, DMSO-d6) δ=−113.26, −119.78.

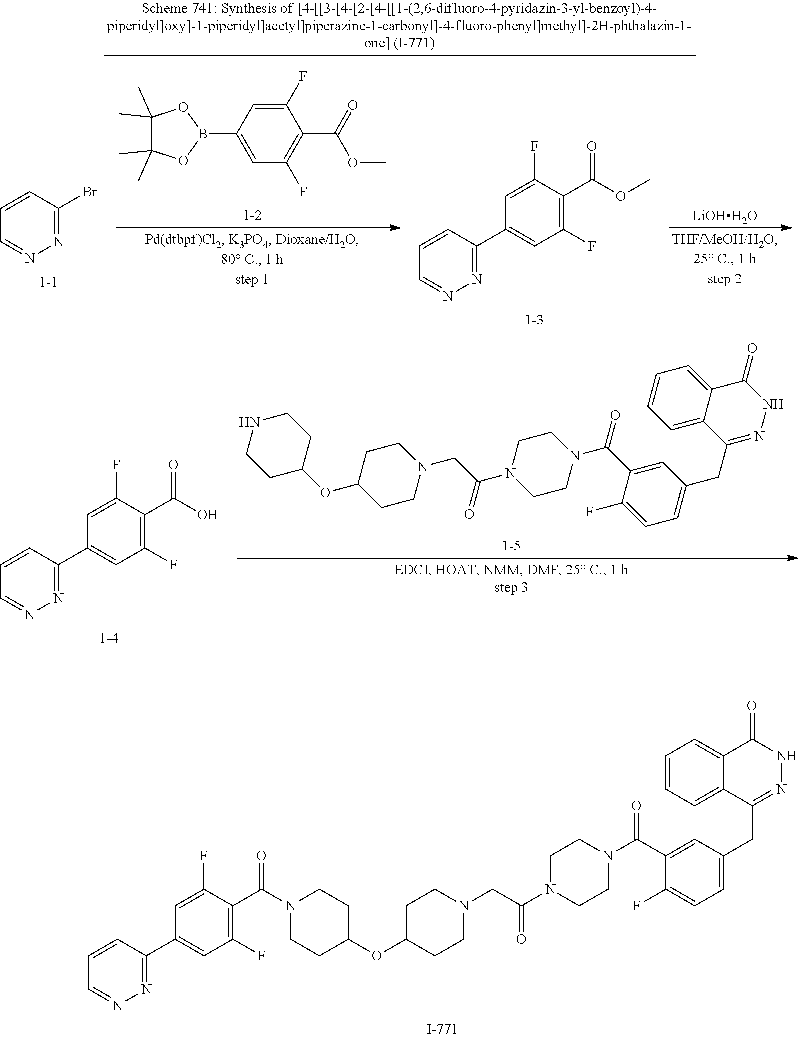

### Step 1: Synthesis of Intermediate 1-3

A mixture of Intermediate 1-2 (374.99 mg, 1.26 mmol, 1 eq), Intermediate 1-1 (200 mg, 1.26 mmol, 1 eq), Pd(dtbpf)Cl₂ (81.99 mg, 125.80 μmol, 0.1 eq), K₃PO₄ (801.10 mg, 3.77 mmol, 3 eq) in dioxane (2 mL)/H₂O (0.4 mL) was degassed and purged with N₂ 3 times, and then the mixture was stirred at 80° C. for 1.5 hr under N₂ atmosphere. The reaction mixture was diluted with water (10 mL) and extracted with DCM (10 mL*3). The combined organic layers were dried over Na₂SO₄, filtered and concentrated under reduced pressure to give a residue. The residue was purified by flash silica gel chromatography (ISCO; 20 g SepaFlash Silica Flash Column, Eluent of 0˜100% Petroleum ether/Ethyl acetate @40 mL/min). The eluent was concentrated under reduced pressure to give a residue. Intermediate 1-3 (390 mg, crude) was obtained as a brown oil. LCMS (Method D): Retention time: 0.322 min, [M+H]⁺=251.0.

### Step 2: Synthesis of Intermediate 1-4

To a solution of Intermediate 1-3 (330 mg, 1.32 mmol, 1 eq) in THF (1.1 mL), H₂O (1.1 mL) and MeOH (1.1 mL) was added LiOH H₂O (110.70 mg, 2.64 mmol, 2 eq). The mixture was stirred at 25° C. for 0.5 hr. The mixture was adjusted to pH=5 with citric acid and filtered and concentrated under reduced pressure to give a residue. The residue was used in the next step without further purification. Intermediate 1-4 (220 mg, 931.52 μmol, 70.63% yield) was obtained as a brown solid. LCMS (Method D): Retention time: 0.164 min, [M+H]⁺=237.0.

### Step 3: Synthesis of I-771

To a solution of Intermediate 1-4 (150 mg, 635.12 μmol, 1 eq) in DMF (2 mL) was added HOAt (86.45 mg, 635.12 μmol, 88.85 μL, 1 eq), EDCI (365.26 mg, 1.91 mmol, 3 eq), NMM (321.20 mg, 3.18 mmol, 349.14 μL, 5 eq) and Intermediate 1-5 (398.32 mg, 635.12 μmol, 1 eq, HCl salt). The mixture was stirred at 25° C. for 1 hr. The reaction mixture was diluted with water (10 mL) and extracted with DCM (5 mL*3). The combined organic layers were dried over Na₂SO₄, filtered and concentrated under reduced pressure to give a residue. The residue was purified by reversed-phase column chromatography (Neutral condition). The eluent was lyophilized to give a product. I-771 (130 mg, 160.72 gmol, 25.31% yield, 100% purity) was obtained as a white solid. LCMS (Method D): Retention time: 0.311 min, [M+H]⁺=809.4. ¹H NMR (400 MHz, METHANOL-d4) δ=9.23-9.22 (m, 1H), 8.38-8.36 (m, 1H), 8.29-8.26 (m, 1H), 7.92 (d, J=9.2 Hz, 3H), 7.90-7.80 (m, 3H), 7.49 (d, J=2.8 Hz, 1H), 7.38 (s, 1H), 7.16 (t, J=9.2 Hz, 1H), 4.39 (s, 2H), 4.11-4.02 (m, 1H), 3.86-3.47 (m, 11H), 3.29-3.12 (m, 4H), 2.77 (d, J=1.2 Hz, 2H), 2.34-2.15 (m, 2H), 2.00-1.83 (m, 4H), 1.71-1.53 (m, 4H). ¹⁹F NMR (376 MHz, METHANOL-d4) δ=−114.21 (s, 1F), −114.32 (s, 1F), −120.78 (br d, J=14.1 Hz, 1F).

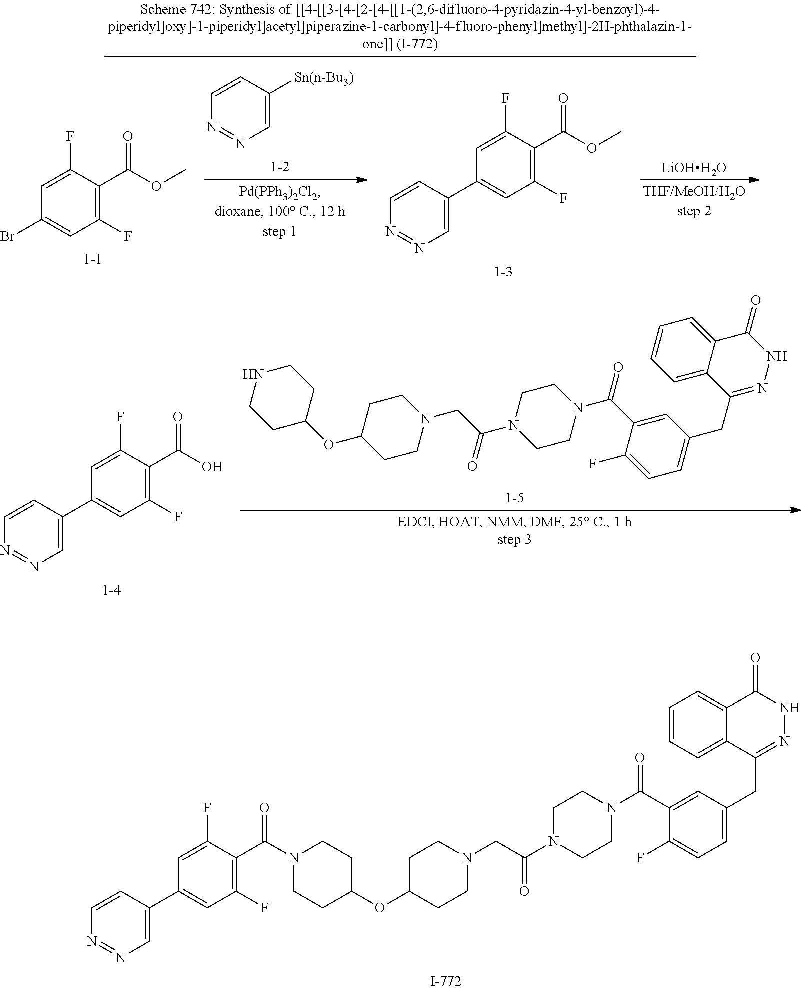

### Step 1: Synthesis of Intermediate 1-3.

A mixture of Intermediate 1-1 (808.77 mg, 2.19 mmol, 1.1 eq), Intermediate 1-2 (808.77 mg, 2.19 mmol, 1.1 eq) and Pd(PPh₃)₂Cl₂ (139.81 mg, 199.18 μmol, 0.1 eq) in dioxane (5 mL) was degassed and purged with N₂ 3 times, and then the mixture was stirred at 100° C. for 1 hr under N₂ atmosphere. The reaction mixture was poured into water (10 mL) and extracted with EA (8 mL*3). The combined organic layers were dried over Na₂SO₄, filtered and concentrated under reduced pressure to give a residue. The residue was purified by flash silica gel chromatography (ISCO; 20 g SepaFlash Silica Flash Column, Eluent of 0˜100% PE/EA) @50 mL/min, PE/EA=2:1, Rf=0.4) and the eluent was concentrated under reduced pressure to give Intermediate 1-3 (580 mg, 1.95 mmol, 97.76% yield, 84% purity) as a yellow solid LCMS (Method D): Retention time: 0.306 min, [M+H]⁺=251.0. ¹H NMR (400 MHz, CHLOROFORM-d) δ=9.44 (d, J=1.2 Hz, 1H), 9.36-9.33 (m, 1H), 7.66-7.62 (m, 1H), 7.31-7.29 (m, 1H), 7.29-7.27 (m, 1H), 4.01 (s, 3H).

### Step 2: Synthesis of Intermediate 1-4.

To a solution of Intermediate 1-3 (500 mg, 2.00 mmol, 1 eq) in THF (2 mL), MeOH (2 mL) and H₂O (2 mL) was added LiOH H₂O (251.58 mg, 6.00 mmol, 3 eq). The mixture was stirred at 25° C. for 0.5 hr. The reaction mixture was adjusted to pH=3 with 1M HCl, diluted with H₂O (10 mL) and then extracted with EA (10 mL*3), dried by Na₂SO₄, filtered and concentrated to give Intermediate 1-4 (500 mg, crude) as a yellow solid. LCMS (Method D): Retention time: 0.186 min, [M+H]⁺=237.0.

### Step 3: Synthesis of I-772.

To a solution of Intermediate 1-4 (200 mg, 846.83 μmol, 1 eq) in DMF (5 mL) was added EDCI (487.02 mg, 2.54 mmol, 3 eq), HOAt (115.26 mg, 846.83 μmol, 118.46 μL, 1 eq), NMM (428.27 mg, 4.23 mmol, 465.51 μL, 5 eq) and Intermediate 1-5 (500.21 mg, 846.83 μmol, 1 eq). The mixture was stirred at 25° C. for 0.5 hr. The reaction mixture was poured into water (15 mL) and extracted with EA (10 mL*3). The combined organic layers were dried over Na₂SO₄, filtered and concentrated under reduced pressure to give a residue. The residue was purified by reverse phase column chromatography (0.1% NH₃·H₂O) and the eluent was lyophilized to give I-772 (159.08 mg, 195.79 μmol, 23.12% yield, 99.55% purity) as a white solid. LCMS (Method D): Retention time: 0.317 min, [M+H]⁺=809.3. HPLC: Retention time: 1.970 min. ¹H NMR (400 MHz, METHANOL-d4) δ=9.60 (d, J=1.2 Hz, 1H), 9.31-9.28 (m, 1H), 8.38-8.35 (m, 1H), 8.08-8.05 (m, 1H), 7.99-7.91 (m, 1H), 7.91-7.79 (m, 2H), 7.72-7.69 (m, 2H), 7.49 (d, J=2.4 Hz, 1H), 7.37 (s, 1H), 7.16 (t, J=9.2 Hz, 1H), 4.38 (s, 2H), 4.11-4.00 (m, 1H), 3.80 (d, J=4.0 Hz, 2H), 3.73 (br s, 2H), 3.66 (s, 2H), 3.56 (s, 3H), 3.51 (d, J=4.4 Hz, 1H), 3.36 (s, 1H), 3.27 (s, 3H), 3.20 (s, 1H), 2.78 (s, 2H), 2.38-2.16 (m, 2H), 1.97-1.81 (m, 4H), 1.70-1.52 (m, 4H)¹⁹FNMR (400 MHz, METHANOL-d4) δ=−113.566, −113.694, −120.749.

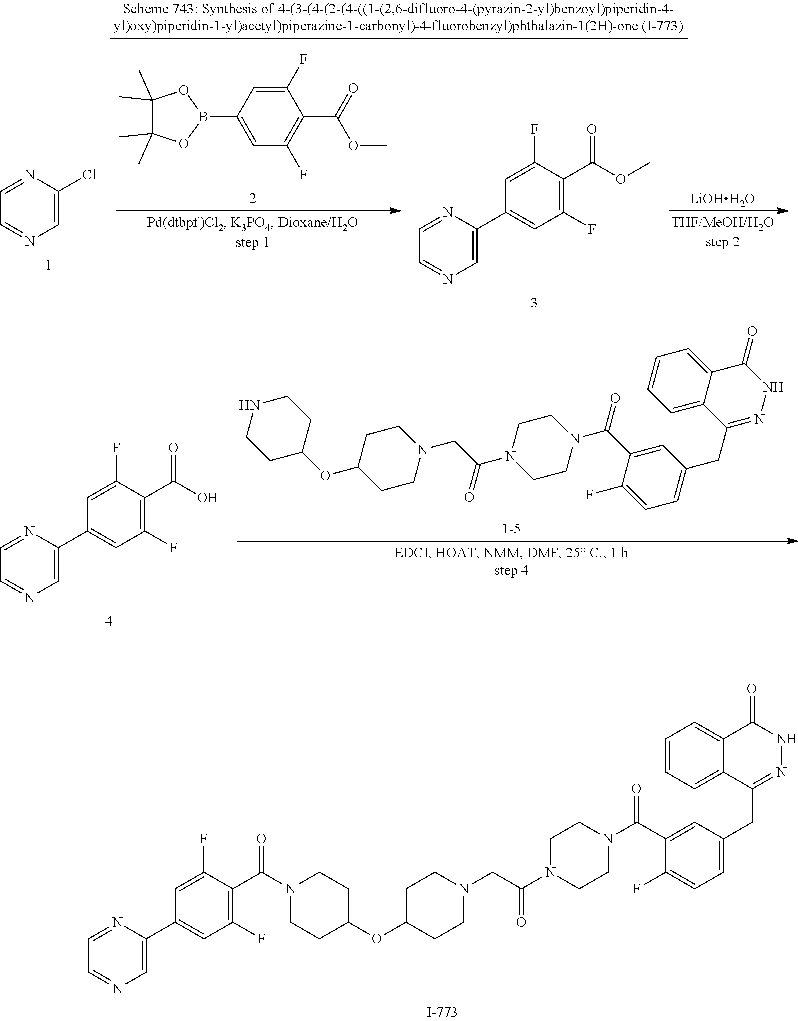

### Step 1: Synthesis of Intermediate 3

A mixture of Intermediate 1 (200 mg, 1.75 mmol, 155.88 uL, 1 eq), Intermediate 2 (520.53 mg, 1.75 mmol, 1 eq), Pd(dppf)Cl₂ (113.81 mg, 174.62 μmol, 0.1 eq) and K₃PO₄ (1.11 g, 5.24 mmol, 3 eq) in dioxane (5 mL) and H₂O (1 mL) was degassed and purged with N₂ 3 times, and then the mixture was stirred at 80° C. for 1 hr under an N₂ atmosphere. The reaction mixture was combined with another lot of material for workup, and the resulting mixture was poured into water (10 mL) and extracted with EA (8 mL*3). The combined organic layers were dried over Na₂SO₄, filtered and concentrated under reduced pressure to give a residue. The residue was purified by flash silica gel chromatography (ISCO; 20 g SepaFlash Silica Flash Column, Eluent of 0˜100% PE/EA)@50 mL/min, PE/EA=2:1, Rf=0.4) and the eluent was concentrated under reduced pressure to give Intermediate 3 (450 mg, crude) as a yellow solid. LCMS (Method D): Rt=0.369 min, [M+H]⁺=251.0.

### Step 2: Synthesis of Intermediate 4

To a solution of Intermediate 3 (350 mg, 1.40 mmol, 1 eq) in THF (1.5 mL), MeOH (1.5 mL) and H₂O (1.5 mL) was added LiOH·H₂O (176.11 mg, 4.20 mmol, 3 eq). The mixture was stirred at 25° C. for 0.5 hr. The reaction mixture was combined with another lot of material for workup. The reaction pH was adjusted to pH=3 with 1M HCl, diluted with H₂O (10 mL), and the mixture was filtered and the solid was dried under reduced pressure to give Intermediate 4 (400 mg, crude) as a yellow solid. LCMS (Method D): Rt=0.269 min, [M+H]⁺=237.0.

### Step 4: Synthesis of I-773

To a solution of Intermediate 4 (200 mg, 846.83 μmol, 1 eq) and Intermediate 5 (531.09 mg, 846.83 μmol, 1 eq, HCl salt) in DMF (2 mL) was added HOAt (172.89 mg, 1.27 mmol, 177.69 μL, 1.5 eq), EDCI (324.68 mg, 1.69 mmol, 2 eq) and NMM (428.27 mg, 4.23 mmol, 465.51 μL, 5 eq). The mixture was stirred at 25° C. for 1 hr. The reaction was quenched by water (1 mL). The mixture was purified by reversed phase HPLC (ISCO; 80 g SepaFlash C18 Column, Eluent of 0˜50%(0.1% FA) water/MeCN@60 mL/min). The product was collected and then repurified by Prep-HPLC (column: Unisil C8;mobile phase: [water(FA)-ACN]; gradient: 10%-40% B over 40 min). The product was further purified by Prep-HPLC (column: Waters Xbridge 150*25 mm*5 um;mobile phase: [water(NH₄HCO₃)-ACN]; gradient: 18%-48% B over 15 min) to give I-773 (161 mg, 198.39 μmol, 23.43% yield, 99.67% purity) as a yellow solid. LCMS (Method E): Rt=0.426 min, [M+H]⁺=809.3. ¹H NMR (400 MHz, DMSO-d6) δ=12.59 (s, 1H), 9.39 (d, J=1.2 Hz, 1H), 8.84-8.75 (m, 1H), 8.72 (d, J=2.4 Hz, 1H), 8.26 (d, J=7.6 Hz, 1H), 8.01 (br d, J=8.8 Hz, 2H), 7.98-7.93 (m, 1H), 7.92-7.86 (m, 1H), 7.86-7.79 (m, 1H), 7.49-7.40 (m, 1H), 7.40-7.32 (m, 1H), 7.23 (m, 1H), 4.33 (s, 2H), 4.10-3.96 (m, 1H), 3.79-3.67 (m, 1H), 3.67-3.53 (m, 3H), 3.53-3.38 (m, 5H), 3.25-3.01 (m, 6H), 2.74-2.59 (m, 2H), 2.18-2.04 (m, 2H), 1.91-1.69 (m, 4H), 1.52-1.28 (m, 4H). F NMR (400 MHz, DMSO-d6) δ=−113.2, −119.8.

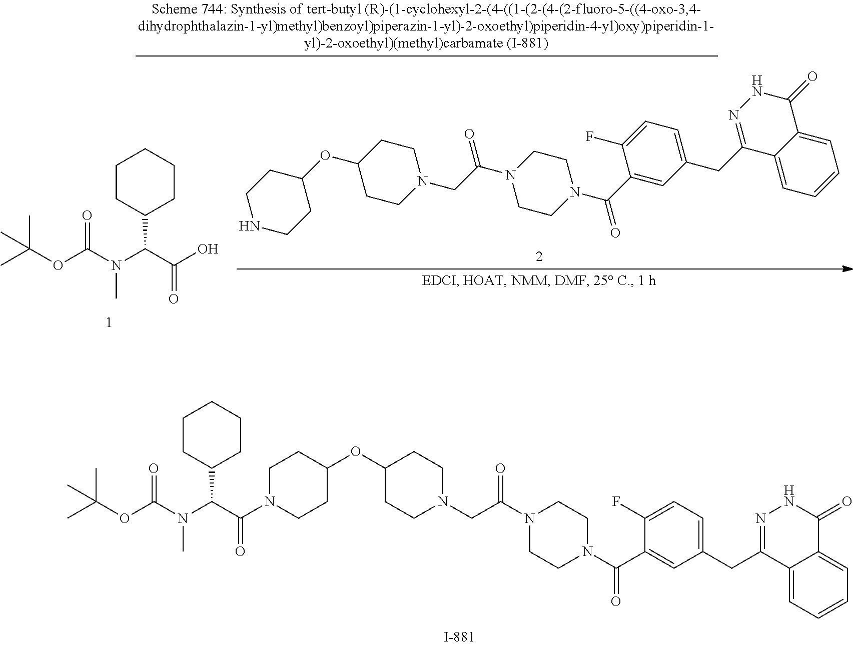 [SMILES: CN(C(=O)OC(C)(C)C)[C@@H](C(=O)O)C1CCCCC1 ; O=C(CN1CCC(OC2CCNCC2)CC1)N1CCN(C(=O)c2cc(Cc3n[nH]c(=O)c4ccccc34)ccc2F)CC1 ; CN(C(=O)OC(C)(C)C)[C@@H](C(=O)N1CCC(OC2CCN(CC(=O)N3CCN(C(=O)c4cc(Cc5n[nH]c(=O)c6ccccc56)ccc4F)CC3)CC2)CC1)C1CCCCC1]

To a solution of Intermediate 1-1 (300 mg, 1.11 mmol, 1 eq) and Intermediate 1-2 (762.70 mg, 1.22 mmol, 1.1 eq, HCl salt) in DMF (3 mL) was added EDCI (635.82 mg, 3.32 mmol, 3 eq), HOAt (150.48 mg, 1.11 mmol, 154.66 μL, 1 eq) and NMM (559.13 mg, 5.53 mmol, 607.75 μL, 5 eq). The mixture was stirred at 25° C. for 0.5 hr. The mixture was diluted with water (10 mL) and extracted with DCM (10 mL*3). The combined organic layer was dried over Na₂SO₄, filtered and concentrated in vacuo to give a residue. The mixture was purified by prep-HPLC (column: CD05-Phenomenex luna C18 150*40*10 um; mobile phase: [water(FA)-ACN]; gradient: 15%-45% B over 10 min). Then the eluent was concentrated in vacuo and lyophilized. I-881 (550 mg, 638.61 μmol, 57.76% yield, 98% purity) was obtained as a white solid. I-881 was further purified by reversed phase chromatography (26.8*125 mm, 40 g of XB—C18, 20-40 m, 120 Å) Mobile phase: A for H₂O (0.1% FA v/v) and B for acetonitrile; Gradient: B 5%-95% in 15 min; Flow rate: 40 mL/min; Column temperature: R.T. Wavelength: 220 nm/254 nm). The target peak was concentrated and lyophilized to afford the desired product. I-881 (294.15 mg, 328.80 μmol, 50.46% yield, 99.49% purity, FA salt) was obtained as a white solid. LCMS (Method D): Rt=0.402 min, (M+H)=844.9. ¹H NMR (400 MHz, DMSO-d6) δ=12.60 (s, 1H), 8.26 (d, J=7.6 Hz, 1H), 8.14 (s, 1H), 8.00-7.94 (m, 1H), 7.89-7.87 (m, 1H), 7.86-7.80 (m, 1H), 7.43 (d, J=5.6 Hz, 1H), 7.39-7.35 (m, 1H), 7.25-7.21 (m, 1H), 4.61-4.42 (m, 1H), 4.33 (s, 2H), 4.14-3.80 (m, 1H), 3.70 (d, J=2.4 Hz, 5H), 3.52-3.36 (m, 5H), 3.19-3.08 (m, 4H), 2.91-2.57 (m, 6H), 2.22-2.06 (m, 2H), 1.97-1.85 (m, 1H), 1.84-1.51 (m, 8H), 1.46-1.30 (m, 13H), 1.26-1.02 (m, 4H), 0.96-0.72 (m, 2H). ¹⁹F NMR (377 MHz, DMSO-d6) δ=−119.68-−119.89 (m, 1F). SFC: Rt=1.998 min, ee value>100.00.

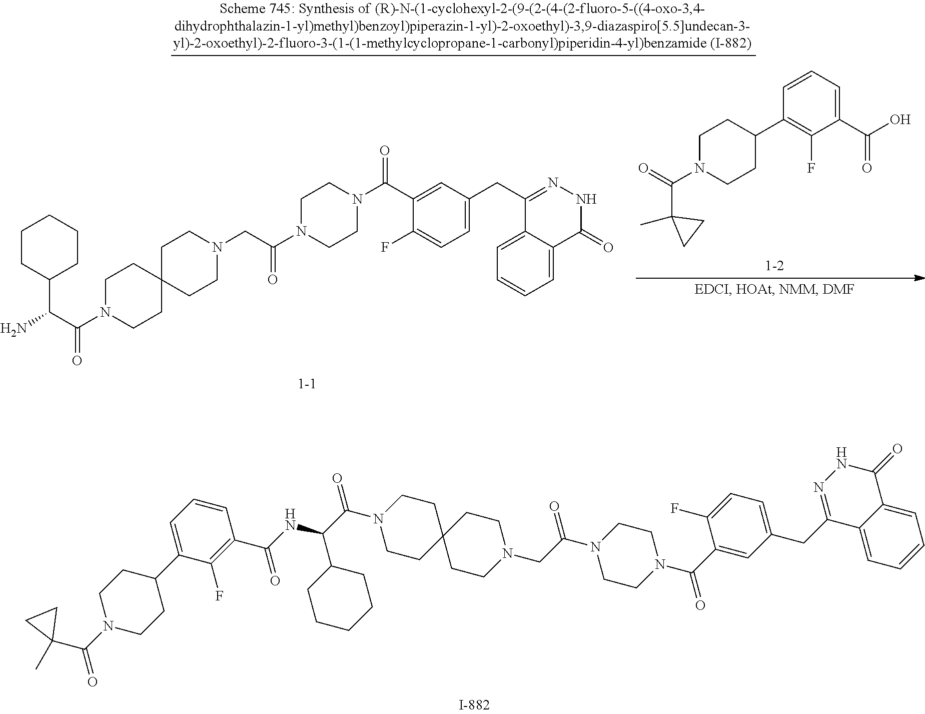

To a solution of Intermediate 1-1 (800 mg, 1.09 mmol, 1 eq, HCl salt) in DMF (8 mL) was added EDCI (624.84 mg, 3.26 mmol, 3 eq), NMM (549.48 mg, 5.43 mmol, 597.26 μL, 5 eq), Intermediate 1-2 (364.93 mg, 1.20 mmol, 1.1 eq) and HOAt (147.88 mg, 1.09 mmol, 151.99 μL, 1 eq). The mixture was stirred at 25° C. for 0.5 h. The reaction mixture was quenched with water (15 mL) and extracted with EA (10 mL*3), combined the organic phase was washed by aqueous NaCl (10 mL*3) and dried by anhydrous Na₂SO₄, and concentrated to give crude product. The crude was purified by prep-HPLC (column: UniSil 3-100 C18 Ultra (150*25 mm*3 um); mobile phase: [water (FA)-ACN]; gradient: 10%-40% B over 40 min), then concentrated to remove organic solvents and then lyophilized to give product. I-882 (185 mg, 187.40 μmol, 18.50% yield) was obtained as a white solid. LCMS (Method E): Retention time=1.192 min, [M+H]⁺=987.7. SFC: Retention time: 2.176 min. ¹H NMR (400 MHz, DMSO-d6) δ=12.59 (s, 1H), 8.38-8.21 (m, 2H), 8.02-7.94 (m, 1H), 7.93-7.80 (m, 2H), 7.51-7.33 (m, 4H), 7.28-7.17 (m, 2H), 4.87-4.73 (m, 1H), 4.41 (d, J=13.2 Hz, 2H), 4.33 (s, 2H), 3.70-3.44 (m, 8H), 3.42-3.36 (m, 2H), 3.21-3.08 (m, 5H), 3.06-2.77 (m, 2H), 2.40-2.31 (m, 4H), 1.84-1.65 (m, 6H), 1.64-1.52 (m, 4H), 1.51-1.29 (m, 8H), 1.24 (s, 3H), 1.22-1.09 (m, 3H), 1.07-0.94 (m, 2H), 0.88-0.77 (m, 2H), 0.59-0.48 (m, 2H). ¹⁹F NMR (377 MHz, DMSO-d6) δ=−119.78, −120.96.

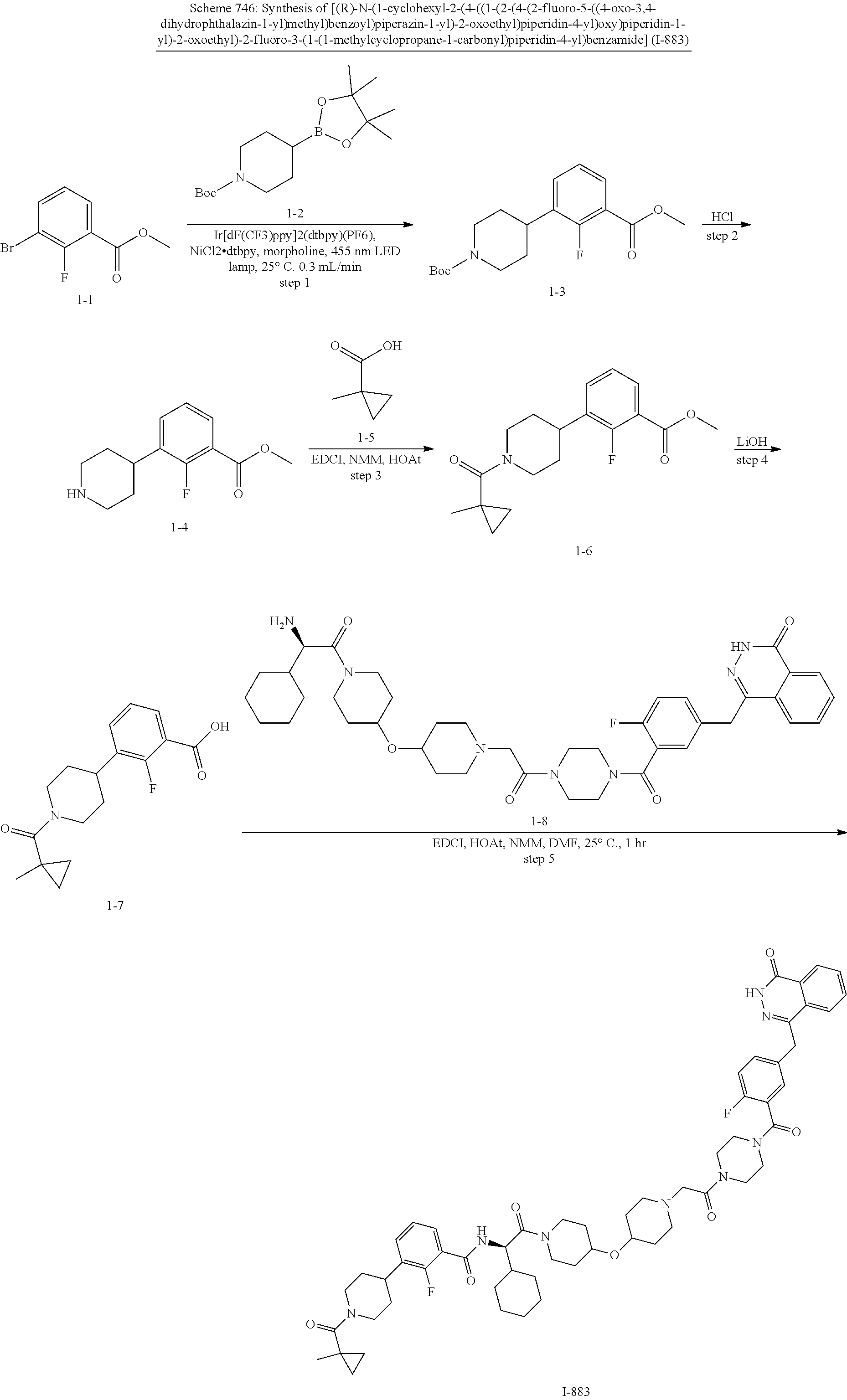

### Step 1: Synthesis of Intermediate 1-3

To an 15 mL vial equipped with a stir bar was added Intermediate 1-1 (5 g, 21.46 mmol, 1 eq), Intermediate 1-2 (13.36 g, 42.91 mmol, 2 eq), Ir[dF(CF₃)ppy]₂(dtbpy)(PF₆), (240.72 mg, 214.56 μmol, 0.01 eq), NiCl₂ dtbpy (426.97 mg, 1.07 mmol, 0.05 eq), and morpholine (2.80 g, 32.18 mmol, 2.83 mL, 1.5 eq) in DMF (100 mL). The reaction solution was pumped through a reactor at a flow rate of 300 L min⁻¹, irradiating with a 455 nm LED lamp, with cooling water to keep the reaction temperature at 25° C. for 0.5 h. The reaction mixture was filtered and concentrated under reduced pressure to give a residue. Then the residue was purified by flash silica gel chromatography (ISCO; 80 g SepaFlash Silica Flash Column, Eluent of 0˜60% Ethyl acetate/Petroleum ether gradient @50 mL/min) and the eluent was concentrated to give Intermediate 1-3 (10 g, crude) as a colorless oil. LCMS (Method D): Retention time: 0.469 min, [M+H]⁺=282.0. ¹H NMR (400 MHz, METHANOL-d4) δ=7.78-7.71 (m, 1H), 7.56-7.49 (m, 1H), 7.24-7.20 (m, 1H), 4.23 (d, J=13.6 Hz, 2H), 3.91-3.89 (m, 3H), 3.18-3.08 (m, 1H), 2.97 (s, 2H), 1.81 (d, J=12.8 Hz, 2H), 1.75-1.67 (m, 2H), 1.48 (s, 9H). ¹⁹F NMR (377 MHz, METHANOL-d4) δ=−118.96.

### Step 2: Synthesis of Intermediate 1-4

A solution of Intermediate 1-3 (4 g, 296.40 μmol, 1 eq) in HCl/dioxane (1 mL) was stirred at 25° C. for 0.5 hr. The reaction mixture was concentrated under reduced pressure to give Intermediate 1-4 (4 g, crude, HCl salt) as a white solid. LCMS (Method D): Retention time: 0.162 min, [M+H]⁺=238.1.

### Step 3: Synthesis of Intermediate 1-6

To a solution of Intermediate 1-4 (1 g, 3.65 mmol, 1 eq, HCl salt) and Intermediate 1-5 (365.75 mg, 3.65 mmol, 1 eq) in DMF (10 mL) was added HOAt (497.24 mg, 3.65 mmol, 511.04 μL, 1 eq), EDCI (2.10 g, 10.96 mmol, 3 eq) and NMM (1.85 g, 18.27 mmol, 2.01 mL, 5 eq). The mixture was stirred at 25° C. for 0.5 hr. The reaction mixture was diluted with H₂O (20 mL) and extracted with DCM (20 mL*3). The combined organic layers were washed with NaCl (aq) (50 mL), dried over Na₂SO₄, filtered and concentrated under reduced pressure to give a residue. The residue was purified by flash silica gel chromatography (ISCO; 40 g SepaFlash Silica Flash Column, Eluent of 0˜100% Ethyl acetate/Petroleum ether gradient @40 mL/min) and concentrated under reduced pressure to give a residue. Intermediate 1-6 (0.7 g, 2.19 mmol, 60.00% yield) was obtained as a yellow oil. ¹H NMR (400 MHz, METHANOL-d4) δ=7.81-7.72 (m, 1H), 7.58-7.51 (m, 1H), 7.25-7.21 (m, 1H), 5.49 (s, 3H), 3.90 (s, 3H), 3.11 (s, 2H), 3.01 (s, 2H), 2.02-1.96 (m, 2H), 1.34 (s, 3H), 0.95-0.91 (m, 2H), 0.67-0.62 (m, 2H). ¹⁹F NMR (376 MHz, METHANOL-d4) δ=−118.94.

### Step 4: Synthesis of Intermediate 1-7

A solution of Intermediate 1-6 (0.65 g, 2.04 mmol, 1 eq) in THF (2.5 mL), MeOH (2.5 mL), H₂O (2.5 mL) was added LiOH·H₂O (256.22 mg, 6.11 mmol, 3 eq) was stirred at 25° C. for 0.5 hr. The reaction mixture was adjusted pH=6 by HCl (2M), and then extracted with DCM (10 mL*2), dried over Na₂SO₄, filtered and concentrated under reduced pressure to give a residue. Intermediate 1-7 (0.17 g, 556.75 mol, 27.36% yield) was obtained as a white solid. LCMS (Method D): Retention time: 0.373 min, [M+H]⁺=306.1. ¹H NMR (400 MHz, METHANOL-d4) δ=7.76-7.72 (m, 1H), 7.54-7.47 (m, 1H), 7.22-7.18 (m, 1H), 4.59 (d, J=13.6 Hz, 2H), 3.26-3.20 (m, 1H), 1.91 (d, J=12.8 Hz, 2H), 1.68 (d, J=12.8 Hz, 2H), 1.34 (s, 3H), 1.26-1.22 (m, 2H), 0.96-0.91 (m, 2H), 0.66-0.62 (m, 2H). ¹⁹F NMR (376 MHz, METHANOL-d4) δ=−119.34 (s, 1F).

### Step 5: Synthesis of I-883

To a solution of Intermediate 1-7 (155.39 mg, 508.91 μmol, 1 eq) and Intermediate 1-8 (0.39 g, 508.91 μmol, 1 eq, HCl salt) in DMF (4 mL) was added EDCI (292.68 mg, 1.53 mmol, 3 eq), HOAt (69.27 mg, 508.91 μmol, 71.19 μL, 1 eq) and NMM (257.37 mg, 2.54 mmol, 279.75 μL, 5 eq). The mixture was stirred at 25° C. for 0.5 hr. The reaction mixture was diluted with H₂O (20 mL) and extracted with DCM (20 mL*2). The combined organic layers were washed with NaCl (aq) (20 mL), dried over Na₂SO₄, filtered and concentrated under reduced pressure to give a residue. The residue was purified by prep-HPLC (column: CD04-Welch Utimate C18 150*25*7 um; mobile phase: [water(FA)-ACN]; gradient: 16%-46% B over 10 min) and the eluent was concentrated to remove ACN and lyophilized to give product. I-883 (228.60 mg, 220.22 μmol, 43.27% yield, 97.99% purity) was obtained as a white solid. LCMS (Method D): Retention time: 0.394 min, [M+H]⁺=1017.5. SFC: Retention time: 1.958 min. ¹H NMR (400 MHz, METHANOL-d4) δ=8.39 (d, J=7.6 Hz, 1H), 7.97 (d, J=7.2 Hz, 1H), 7.93-7.81 (m, 2H), 7.61-7.45 (m, 3H), 7.44-7.37 (m, 1H), 7.27-7.23 (m, 1H), 7.21-7.16 (m, 1H), 4.99 (d, J=7.6 Hz, 1H), 4.61 (d, J=12.4 Hz, 2H), 4.41 (s, 2H), 4.05 (d, J=6.4 Hz, 1H), 3.97-3.75 (m, 4H), 3.75-3.58 (m, 4H), 3.57-3.47 (m, 4H), 3.42 (d, J=0.8 Hz, 2H), 3.31-3.22 (m, 2H), 2.96-2.93 (m, 3H), 2.63-2.43 (m, 2H), 2.05-1.63 (m, 18H), 1.59-1.46 (m, 1H), 1.35 (s, 3H), 1.34-1.09 (m, 5H), 1.00-0.90 (m, 2H), 0.71-0.63 (m, 2H). ¹⁹F NMR (377 MHz, METHANOL-d4) δ=−120.75, −122.41.

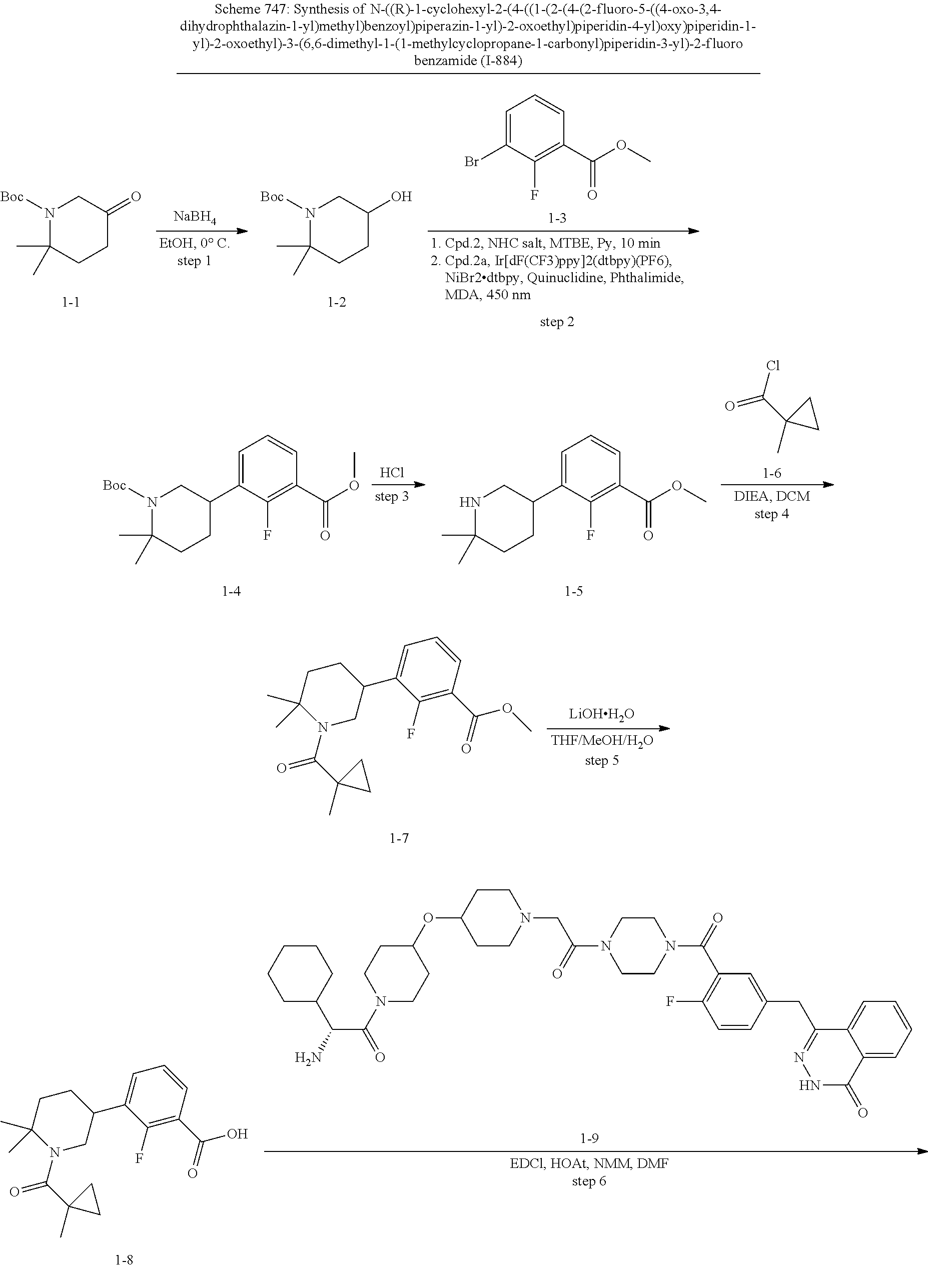

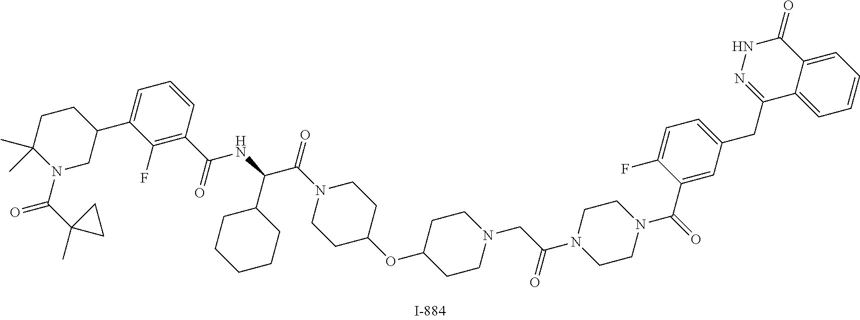 [SMILES: CC1(C(=O)N2CC(c3cccc(C(=O)N[C@@H](C(=O)N4CCC(OC5CCN(CC(=O)N6CCN(C(=O)c7cc(Cc8n[nH]c(=O)c9ccccc89)ccc7F)CC6)CC5)CC4)C4CCCCC4)c3F)CCC2(C)C)CC1]

### Step 1: Synthesis of Intermediate 1-2

To a solution of Intermediate 1-1 (0.84 g, 3.70 mmol, 1 eq) in EtOH (17 mL) was added NaBH₄ (279.61 mg, 7.39 mmol, 2 eq) at 0° C. Then mixture was stirred at 0° C. for 2 hr. The mixture was quenched with a sat. NH₄Cl solution (40 mL), extracted with EA (20 ml*3), and the combined organic layers were washed with brine (20 mL*3), dried over anhydrous Na₂SO₄, filtered and the filtrate was concentrated under vacuum to give Intermediate 1-2 (0.72 g) as a white solid. ¹H NMR (400 MHz, CHLOROFORM-d) δ=3.95-3.87 (m, 1H), 3.66 (dd, J=4.2, 13.6 Hz, 1H), 3.27 (dd, J=7.0, 13.6 Hz, 1H), 1.98-1.92 (m, 1H), 1.73-1.64 (m, 1H), 1.59-1.48 (m, 2H), 1.46 (s, 9H), 1.40 (d, J=1.5 Hz, 6H).

### Step 2: Synthesis of Intermediate 1-4

An 8 mL oven-dried vial was charged with Intermediate 1-2 (714.41 mg, 3.12 mmol, 1.65 eq), NHC-1 (1.19 g, 3.02 mmol, 1.6 eq, CAS: 1207294-92-5). After the vial was vacuumed and refilled with nitrogen gas twice, MTBE (16 mL) was added and the reaction was stirred at 25° C. for 5 min. Then, a pyridine (238.96 mg, 3.02 mmol, 243.84 μL, 1.6 eq) solution in MTBE (4 mL) was added dropwise at 25° C. over the course of 2 min. The resulting solution was stirred at 25° C. for 10 min. A white solid precipitated during this time. Another 15 mL oven-dried vial was charged with Ir(ppy)₂(dtbpy)(PF₆) (25.88 mg, 28.32 μmol, 0.015 eq), NiBr₂-dtbpy (68.95 mg, 141.61 μmol, 0.075 eq), quinuclidine (367.38 mg, 3.30 mmol, 1.75 eq), Intermediate 1-3 (0.44 g, 1.89 mmol, 1 eq) and phthalimide (62.51 mg, 424.83 μmol, 0.225 eq). DMA (20 mL) was added to this vial under an atmosphere of nitrogen. The MTBE suspension was transferred to a 5 mL syringe under air. Then a syringe filter and new needle were installed on the syringe, before the MTBE solution was injected through the syringe filter into the DMA solution. The reaction mixture was sparged with nitrogen for 15 minutes before sealing with parafilm. The vial was stirred at 1500 rpm stir rate and irradiated under 450 nm LED modules at 100% light intensity with maxed fan speed and 1500 rpm stirring rate in a PennOC Integrated Photoreactor for 16 hours. The reaction mixture was quenched by H₂O (40 mL), and extracted with EA (20 mL*3). The combined organic layers were washed with brine (20 mL*3), dried over anhydrous Na₂SO₄, filtered and concentrated under reduced pressure to give a residue. The residue was purified by flash silica gel chromatography (ISCO; 12 g SepaFlash Silica Flash Column, Eluent of 0˜8% Ethyl acetate/Petroleum ether gradient @40 mL/min) and concentrated under vacuum. Intermediate 1-4 (0.33 g, 903.03 μmol, 47.83% yield) was obtained as a yellow oil. LCMS (Method E): Retention time: 0.682 min, [M+H]⁺=266.2. ¹H NMR (400 MHz, CHLOROFORM-d) δ=7.82-7.75 (m, 1H), 7.53-7.45 (m, 1H), 7.15 (t, J=7.7 Hz, 1H), 3.93 (s, 3H), 3.32-3.16 (m, 2H), 2.05-1.91 (m, 1H), 1.77-1.61 (m, 4H), 1.55 (s, 3H), 1.48-1.48 (m, 1H), 1.47-1.45 (m, 9H), 1.39 (s, 3H). ¹⁹F NMR (377 MHz, CHLOROFORM-d) δ=−115.46.

### Step 3: Synthesis of Intermediate 1-5

To a solution of Intermediate 1-4 (0.33 g, 903.03 μmol, 1 eq) in DCM (3 mL) was added HCl/dioxane (4 M, 1 mL, 4.43 eq). The mixture was stirred at 25° C. for 0.5 h. The mixture was concentrated under reduced pressure to give Intermediate 1-5 (0.24 g, crude, HCl salt) as a yellow solid. LCMS (Method G): Retention time: 0.644 min, [M+H]⁺=266.2.

### Step 4: Synthesis of Intermediate 1-7

To a solution of Intermediate 1-5 (0.24 g, 795.27 μmol, 1 eq, HCl salt) and DIEA (308.35 mg, 2.39 mmol, 415.56 μL, 3 eq) in DCM (3 mL) was added Intermediate 1-6 (103.72 mg, 874.80 μmol, 1.1 eq) at 0° C. Then the mixture was stirred at 0° C. for 0.5 h. The reaction mixture was quenched by H₂O (3 mL) and extracted with DCM (3 mL*3). The combined organic layers were washed with brine (3 mL*3), dried over anhydrous Na₂SO₄, filtered and concentrated under reduced pressure to give a residue. The residue was purified by flash silica gel chromatography (ISCO; 12 g SepaFlash Silica Flash Column, Eluent of 0-10% Ethyl acetate/Petroleum ether gradient @40 mL/min) and concentrated under vacuum. Intermediate 1-7 was obtained as a colorless oil. LCMS (Method G): Retention time: 0.687 min, [M+H]⁺=348.2. ¹H NMR (400 MHz, CHLOROFORM-d) δ=7.83 (t, J=7.3 Hz, 1H), 7.47 (t, J=7.0 Hz, 1H), 7.21 (t, J=7.8 Hz, 1H), 4.19 (dd, J=3.9, 13.5 Hz, 1H), 3.95 (s, 3H), 3.26 (tt, J=5.1, 10.3 Hz, 1H), 3.16-3.05 (m, 1H), 1.99-1.75 (m, 4H), 1.71-1.64 (m, 1H), 1.59 (s, 3H), 1.39 (s, 3H), 1.29 (s, 3H), 1.11 (td, J=5.0, 9.8 Hz, 1H), 0.74 (td, J=5.0, 9.8 Hz, 1H), 0.58-0.51 (m, 1H), 0.50-0.43 (m, 1H). ¹⁹F NMR (377 MHz, CHLOROFORM-d) δ=−115.76.

### Step 5: Synthesis of Intermediate 1-8

To a solution of Intermediate 1-7 (0.2 g, 575.67 μmol, 1 eq) in THF (1 mL), MeOH (0.5 mL) and H₂O (1 mL) was added LiOH·H₂O (72.47 mg, 1.73 mmol, 3 eq). The mixture was stirred at 25° C. for 0.5 h. The mixture was concentrated under vacuum to remove THF and MeOH, and the aqueous was adjusted to pH=2, then filtered and the filter cake was washed with H₂O (2 mL*3) and concentrated under vacuum to give Intermediate 1-8 (220 mg, crude) as a white solid. LCMS (Method E): Retention time: 0.517 min, [M+H]⁺=334.1. ¹H NMR (400 MHz, CHLOROFORM-d) δ=7.92-7.82 (m, 1H), 7.45 (t, J=6.4 Hz, 1H), 7.19-7.15 (m, 1H), 4.15 (dd, J=3.6, 13.4 Hz, 1H), 3.30-3.16 (m, 1H), 3.11-3.00 (m, 1H), 1.95-1.71 (m, 3H), 1.61 (ddd, J=4.6, 9.4, 13.8 Hz, 1H), 1.53 (s, 3H), 1.33 (s, 3H), 1.22 (s, 3H), 1.05 (td, J=4.9, 9.9 Hz, 1H), 0.69 (td, J=4.9, 9.9 Hz, 1H), 0.52-0.38 (m, 2H). ¹⁹F NMR (377 MHz, CHLOROFORM-d) δ=−114.59.

### Step 6: Synthesis of I-884

To a solution of Intermediate 1-8 (200 mg, 599.89 μmol, 1 eq) in DMF (2 mL) was added HOAt (81.65 mg, 599.89 μmol, 83.92 μL, 1 eq), EDCI (230.00 mg, 1.20 mmol, 2 eq) NMM (303.38 mg, 3.00 mmol, 329.76 μL, 5 eq) and Intermediate 1-9 (459.72 mg, 599.89 μmol, 1 eq, HCl salt). The mixture was stirred at 25° C. for 1 h. The mixture was poured onto H₂O (10 mL), filtered and the filter cake was washed with H₂O (5 mL*3) and concentrated under vacuum to give a crude product. The crude was purified by Prep-HPLC (column: Waters Xbridge Prep OBD C18 150*40 mm*10 um; mobile phase: [water(NH₄HCO₃)-ACN]; gradient: 42%-72% B over 15 min). I-884 (336.96 mg, 313.92 μmol, 52.33% yield, 97.38% purity) was obtained as a white solid. LCMS (Method G): Retention time=0.738 min, [M+H]⁺=1045.6. SFC: Retention time=2.537, 2.868 min ¹H NMR (400 MHz, METHANOL-d4) δ=8.38 (br d, J=7.6 Hz, 1H), 7.97 (br d, J=7.6 Hz, 1H), 7.93-7.82 (m, 2H), 7.58 (q, J=6.8 Hz, 2H), 7.50 (br s, 1H), 7.40 (br d, J=4.8 Hz, 1H), 7.31-7.29 (m, 1H), 7.18-7.16 (m, 1H), 5.00 (br d, J=7.2 Hz, 1H), 4.40 (s, 2H), 4.26-4.24 (m, 1H), 4.06 (br d, J=5.6 Hz, 1H), 3.98-3.42 (m, 12H), 3.31-3.19 (m, 3H), 2.79 (br d, J=1.6 Hz, 2H), 2.29 (br d, J=7.2 Hz, 2H), 2.10-1.51 (m, 22H), 1.44 (s, 3H), 1.37-1.25 (m, 6H), 1.24-1.07 (m, 3H), 1.03-1.02 (m, 1H), 0.81-0.74 (m, 1H), 0.62-0.52 (m, 2H). ¹⁹F NMR (377 MHz, METHANOL-d4) δ=−120.7, −121.4 (m, 1F).

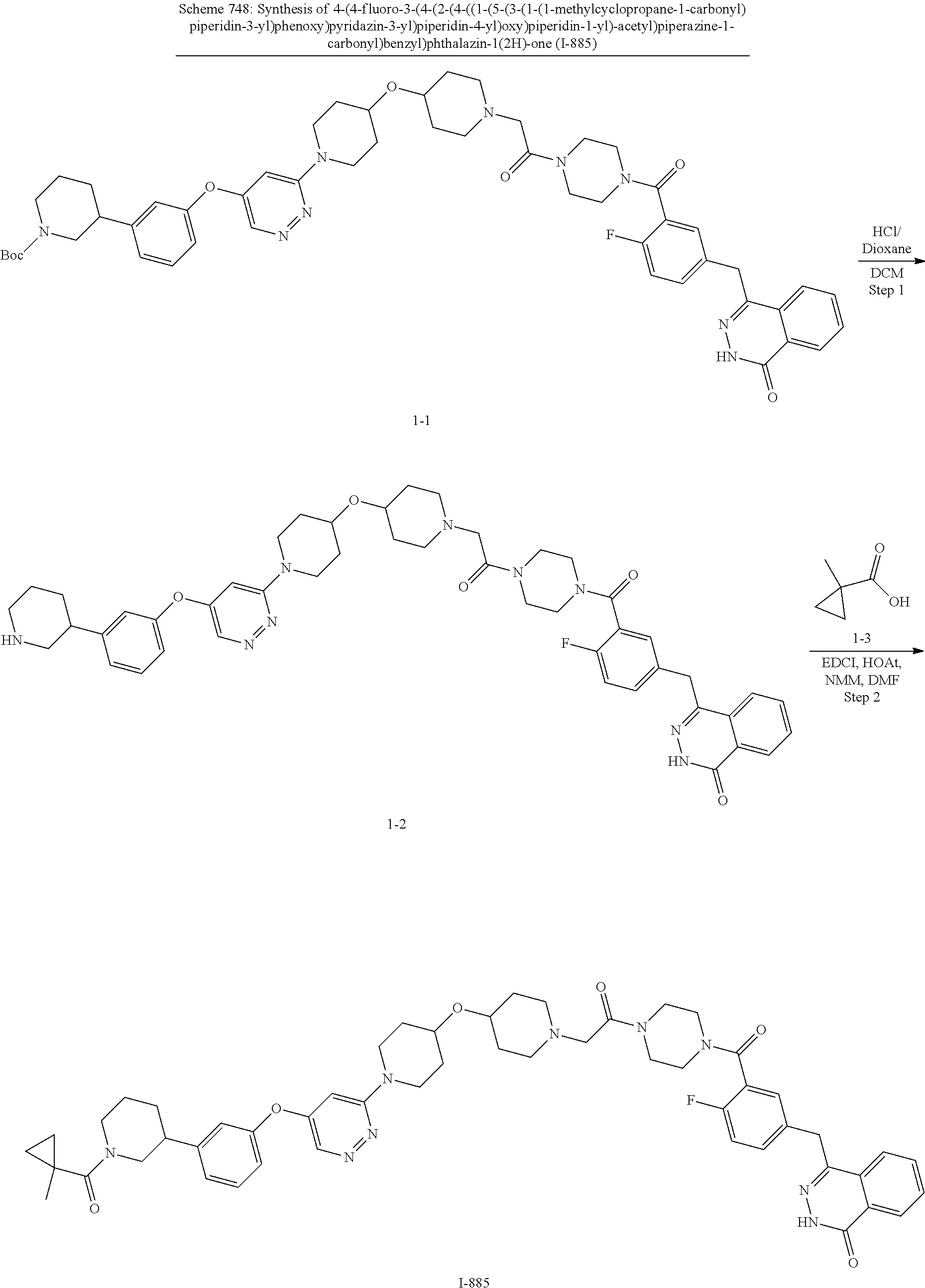

### Step 1: Synthesis of Intermediate 1-2

A solution of Intermediate 1-1 (320 mg, 338.95 μmol, 1 eq) in HCl/dioxane (2 M, 3.20 mL) was stirred for 1 h at 25° C. The reaction mixture was concentrated under reduced pressure to give Intermediate 1-2 (600 mg, crude, HCl salt) as a white solid, the crude product was used in the next step without further purification. LCMS (Method D): Rt=0.261 min, [M+H]⁺=844.4.

### Step 2: Synthesis of I-885

To a solution of Intermediate 1-2 (600 mg, 681.47 μmol, 1 eq, HCl salt) in DMF (6 mL) was added NMM (344.64 mg, 3.41 mmol, 374.61 μL, 5 eq) at 25° C., and the mixture was stirred at 25° C. for 10 min. Then EDCI (653.20 mg, 3.41 mmol, 5 eq), HOAt (185.51 mg, 1.36 mmol, 190.66 μL, 2 eq) and Intermediate 1-3 (88.69 mg, 885.91 μmol, 1.3 eq) were added at 25° C. The resulting mixture was stirred at 25° C. for 1 hr. The reaction mixture was washed with water (6 mL) and extracted with DCM (6 mL*3), the combined organic phase was dried by Na₂SO₄, concentrated under reduced pressure to give a residue. The residue was purified by reversed-phase HPLC (0.1% FA condition) and the eluent was lyophilized to give I-885 (105.73 mg, 108.76 μmol, 50.36% yield, 100% purity, FA salt) as a yellow solid. LCMS (Method E): Retention time=0.442 min, [M+H]⁺=926.6. ¹H NMR (400 MHz, DMSO-d6) δ=12.60 (s, 1H), 8.33 (d, J=2.0 Hz, 1H), 8.26 (m, 1H), 8.17 (s, 1H), 8.00-7.94 (m, 1H), 7.89 (m, 1H), 7.86-7.78 (m, 1H), 7.47-7.34 (m, 3H), 7.27-7.18 (m, 2H), 7.14 (s, 1H), 7.06 (m, 1H), 6.61 (s, 1H), 4.33 (s, 2H), 4.31-4.21 (m, 2H), 4.00-3.88 (m, 2H), 3.71-3.54 (m, 4H), 3.52-3.43 (m, 3H), 3.25-3.06 (m, 7H), 2.97-2.84 (m, 1H), 2.78-2.57 (m, 4H), 2.19-2.05 (m, 2H), 1.96-1.87 (m, 1H), 1.86-1.67 (m, 6H), 1.50-1.32 (m, 5H), 1.21 (s, 3H), 0.86-0.70 (m, 2H), 0.56-0.47 (m, 2H). ¹⁹F NMR (377 MHz, DMSO-d6) δ=−119.79 (brs, 1F). SFC: Retention time=2.097 min, 2.605 min.

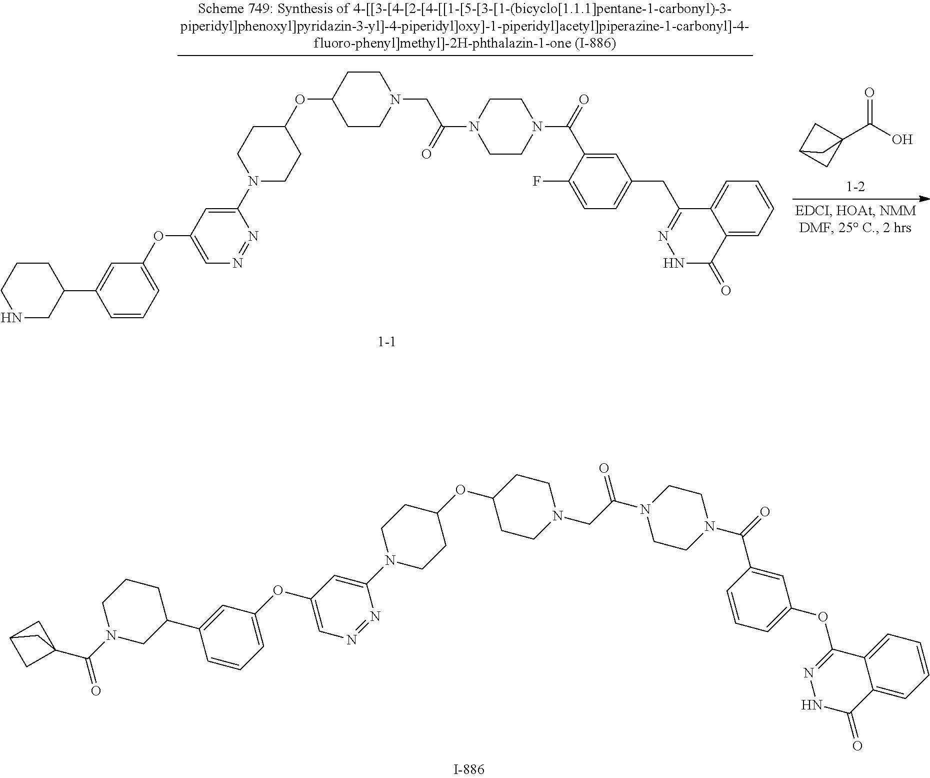 [SMILES: O=C(CN1CCC(OC2CCN(c3cc(Oc4cccc(C5CCCNC5)c4)cnn3)CC2)CC1)N1CCN(C(=O)c2cc(Cc3n[nH]c(=O)c4ccccc34)ccc2F)CC1 ; O=C(O)C12CC(C1)C2 ; O=C(CN1CCC(OC2CCN(c3cc(Oc4cccc(C5CCCN(C(=O)C67CC(C6)C7)C5)c4)cnn3)CC2)CC1)N1CCN(C(=O)c2cccc(Oc3n[nH]c(=O)c4ccccc34)c2)CC1]

To a mixture of Intermediate 1-1 (250 mg, 283.95 μmol, 1 eq, HCl salt) in DMF (2.5 mL) was added Intermediate 1-2 (38.21 mg, 340.74 μmol, 1.2 eq), EDCI (163.3 mg, 851.84 μmol, 3 eq), HOAt (38.65 mg, 283.95 μmol, 39.72 μL, 1 eq) and NMM (143.60 mg, 1.42 mmol, 156.09 μL, 5 eq) at 25° C., then the mixture was stirred at 25° C. for 1 hr. The reaction mixture was diluted with H₂O (5 mL) and extracted with DCM (5 mL×3), and the combined organic layers were dried over Na₂SO₄, filtered and concentrated to give a residue. The residue was purified by reversed phase column chromatography (0.1% FA condition) and lyophilized to give the product. I-886 (250 mg, 245.17 μmol, 86.34% yield, 96.51% purity, FA salt) was obtained as a white solid. The product was further purified by Prep-HPLC with the following condition: column: UniSil 3-100 C18 Ultra (150*25 mm*3 um); mobile phase: [water (FA)-ACN]; gradient: 10%-40% B over 40 min. The major peak was collected and lyophilized to get ˜100 mg of product as a white solid and 70 mg of product with 97% of purity. Further purification by Prep-HPLC with the following condition (column: Waters Xbridge 150*25 mm*5 um; mobile phase: [water (ammonia hydroxide v/v)-ACN]; gradient: 30%-60% B over 12 min). The eluent was concentrated and diluted with 0.10% FA solution (20 mL), then lyophilized to get ˜45 mg of product. Two batches were combined and re-lyophilized to give I-886 (142.32 mg, 144.49 μmol, 54.22% yield, 99.91% purity, FA) as a white solid. LCMS (Method E): Rt=0.444 min, (M+H)=938.6. ¹H NMR (400 MHz, DMSO-d₆) δ=12.59 (s, 1H), 8.35-8.31 (m, 1H), 8.27-8.25 (m, 1H), 8.18 (s, 1H), 8.00-7.94 (m, 1H), 7.92-7.86 (m, 1H), 7.86-7.79 (m, 1H), 7.48-7.33 (m, 3H), 7.29-7.16 (m, 2H), 7.16-7.01 (m, 2H), 6.59 (d, J=3.6 Hz, 1H), 4.41-4.23 (m, 3H), 4.11 (t, J=14.4 Hz, 1H), 4.01-3.85 (m, 2H), 3.72-3.55 (m, 4H), 3.53-3.42 (m, 3H), 3.25-3.00 (m, 8H), 2.74-2.58 (m, 4H), 2.45-2.39 (m, 1H), 2.21-1.97 (m, 8H), 1.95-1.64 (m, 7H), 1.39 (d, J=4.0 Hz, 5H). ¹⁹F NMR (377 MHz, DMSO-d₆) δ=−119.78 (d, J=4.5 Hz, 1F).

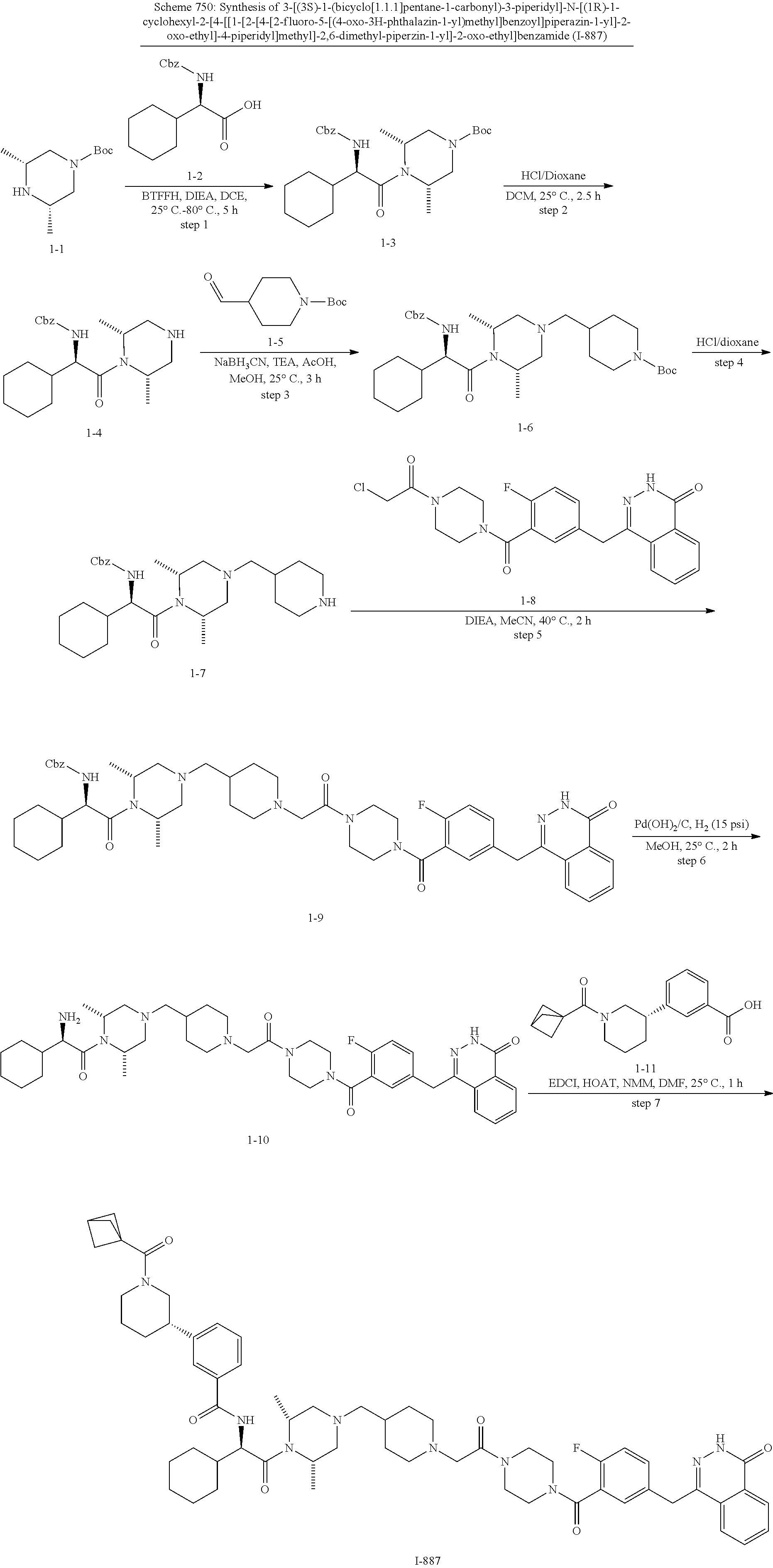

### Step 1: Synthesis of Intermediate 1-3

A mixture of Intermediate 1-2 (2.41 g, 8.27 mmol, 2 eq) and BTFFH (5.23 g, 16.54 mmol, 4 eq) in DCE (9 mL) under N₂ was added DIEA (2.67 g, 20.68 mmol, 3.60 mL, 5 eq), and the mixture was stirred under N₂ at 25° C. for 30 min. The Intermediate 1-1 (886.37 mg, 4.14 mmol, 1 eq) was added and the mixture was heated and stirred at 80° C. for 5 h. The reaction mixture was diluted with water (10 mL) and extracted with DCM (10 mL*3). The combined organic layers were dried over Na₂SO₄, filtered and concentrated under reduced pressure to give a residue. The crude product was purified by reversed-phase column chromatogrpahy (0.1% FA condition) and concentrated under reduced pressure to give a residue. Intermediate 1-3 (2.5 g, crude) was obtained as a yellow oil. LCMS (Method D): Retention time: 0.508 min, [M-Boc+H]⁺=388.2. SFC: Retention time: 0.776 min, 1.049 min ¹H NMR (400 MHz, METHANOL-d4) δ=7.38-7.28 (m, 5H), 5.08 (s, 2H), 4.70-4.42 (m, 2H), 4.28-4.15 (m, 1H), 3.16-2.81 (m, 2H), 1.90-1.70 (m, 4H), 1.69-1.54 (m, 3H), 1.49 (s, 9H), 1.34 (d, J=7.2 Hz, 3H), 1.30-1.13 (m, 7H), 1.12-0.95 (m, 2H).

### Step 2: Synthesis of Intermediate 1-4

To a solution of Intermediate 1-3 (1.2 g, 2.46 mmol, 1 eq) in DCM (6 mL) was added HCl/dioxane (2 M, 12.00 mL, 9.75 eq). The mixture was stirred at 25° C. for 1 hr. The reaction mixture was concentrated under reduced pressure to give a residue. The crude product was used in the next step without further purification. Intermediate 1-4 (1.4 g, crude, HCl salt) was obtained as a yellow solid. LCMS (Method D): Retention time: 0.329 min, [M+H]⁺=388.3.

### Step 3: Synthesis of Intermediate 1-6

To a solution of Intermediate 1-4 (1.4 g, 3.30 mmol, 1 eq, HCl salt) in MeOH (14 mL) was added TEA (2.00 g, 19.81 mmol, 2.76 mL, 6 eq) and it was stirred at 25° C. for 10 min. Then to the mixture was added AcOH (1.19 g, 19.81 mmol, 1.13 mL, 6 eq), then to the mixture was added Intermediate 1-5 (845.09 mg, 3.96 mmol, 1.2 eq). The mixture was stirred at 25° C. for 30 min. To the mixture was added NaBH₃CN (830.04 mg, 13.21 mmol, 4 eq) and it was stirred at 25° C. for 1 hr. The reaction mixture was extracted with DCM (15 mL*3). The combined organic layers were dried over Na₂SO₄, filtered and concentrated under reduced pressure to give a residue. The crude product was purified by reversed-phase column chromatography (0.1% FA condition) and concentrated under reduced pressure to give a residue. Intermediate 1-6 (845 mg, 1.36 mmol, 41.13% yield, 94% purity) was obtained as a white solid. LCMS (Method D): Retention time: 0.396 min, [M+H]⁺=585.8.

### Step 4: Synthesis of Intermediate 1-7

To a solution of intermediate 1-6 (700 mg, 1.20 mmol, 1 eq) in DCM (5 mL) was added HCl/dioxane (2 M, 5 mL, 8.35 eq). The mixture was stirred at 25° C. for 0.5 hr. The reaction mixture was concentrated in vacuo to give a residue. The residue was used in the next step directly without further purification. Intermediate 1-7 (920 mg, crude, HCl salt) was obtained as a white solid. LCMS (Method D): Retention time: 0.274 min, [M+H]⁺=485.4.

### Step 5: Synthesis of Intermediate 1-9

A solution of intermediate 1-7 (900 mg, 1.73 mmol, 1.5 eq, HCl salt) and intermediate 1-8 (509.89 mg, 1.15 mmol, 1 eq) in ACN (9 mL) was added DIEA (446.40 mg, 3.45 mmol, 601.62 μL, 3 eq) was stirred at 40° C. for 2 hrs. The mixture was diluted with water (15 mL) and extracted with DCM (15 mL*3). The combined organic layer was dried over Na₂SO₄, filtered and concentrated in vacuo to give a residue. The residue was purified by column chromatography (SiO₂, DCM:MeOH=20:1 to DCM:MeOH=0:1), then the organic liquid was concentrated in vacuo to give a residue. Intermediate 1-9 (900 mg, 959.51 μmol, 83.34% yield, 95% purity) was obtained as a white solid. LCMS (Method D): Retention time: 0.350 min, [M+H]⁺=891.6.

### Step 6: Synthesis of Intermediate 1-9

To a solution of intermediate 1-8 (900 mg, 1.01 mmol, 1 eq) in MeOH (15 mL) was added Pd(OH)₂/C (200 mg, 1.01 mmol, 20% purity, 1 eq) under N₂ atmosphere. The suspension was degassed and purged with H₂ 3 times. The mixture was stirred under H₂ (15 Psi) at 25° C. for 2 hr. The reaction mixture was filtered and concentrated in vacuo to give a residue. The residue was used in the next step directly without further purification. Intermediate 1-9 (570 mg, 753.02 μmol, 74.56% yield) was obtained as a yellow oil. LCMS (Method D): Retention time: 0.415 min, [M+H]⁺=585.6.

### Step 7: Synthesis of I-887

To a solution of intermediate 1-10 (30 mg, 39.63 μmol, 1 eq) and intermediate 1-11 (14.24 mg, 47.56 μmol, 1.2 eq) in DMF (1 mL) was added EDCI (22.79 mg, 118.90 μmol, 3 eq) NMM (32.07 mg, 317.06 μmol, 34.86 μL, 8 eq) and HOAt (5.39 mg, 39.63 μmol, 5.54 μL, 1 eq). The mixture was stirred at 25° C. for 1 hr. The mixture was concentrated under reduced pressure to give a residue. The residue was purified by flash silica gel chromatography (ISCO; 40 g SepaFlash C18 Column, Eluent of 0˜60% Ethyl acetate/Petroleum ether gradient @40 mL/min). I-887 (24 mg, 22.13 μmol, 55.85% yield, FA salt) was obtained as a white solid. LCMS (Method E): Retention time: 0.485 min, [M+H]⁺=585.6. ¹H NMR (400 MHz, DMSO-d6) δ=12.70 (s, 1H), 8.59 (s, 1H), 8.33 (s, 1H), 8.26 (d, J=7.6 Hz, 1H), 8.00-7.86 (m, 2H), 7.86-7.72 (m, 3H), 7.50-7.33 (m, 4H), 7.26-7.21 (m, 1H), 4.88-4.36 (m, 3H), 4.33 (s, 2H), 4.26-4.05 (m, 2H), 3.67-3.60 (m, 2H), 3.59-3.45 (m, 4H), 3.22-3.02 (m, 6H), 2.84-2.58 (m, 6H), 2.47-2.37 (m, 2H), 2.14-2.06 (m, 6H), 2.04-1.89 (m, 6H), 1.82-1.74 (m, 3H), 1.68 (s, 4H), 1.61 (d, J=1.6 Hz, 2H), 1.52-1.41 (m, 2H), 1.38 (br s, 3H), 1.25-0.87 (m, 10H). ¹⁹F NMR (377 MHz, DMSO-d6) δ=−119.78.

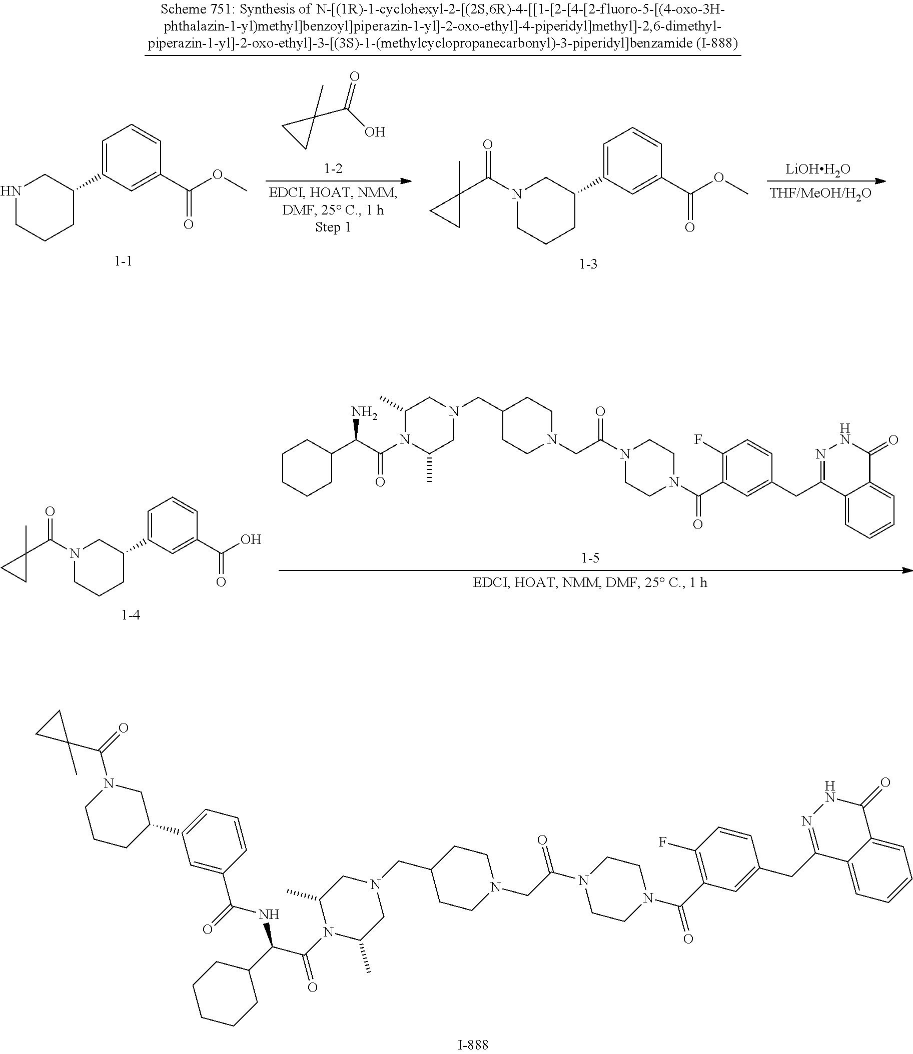

### Step 1: Synthesis of Intermediate 1-3

To a solution of Intermediate 1-1 (165 mg, 645.19 μmol, 1 eq, HCl salt) and Intermediate 1-2 (77.51 mg, 774.22 μmol, 1.2 eq) in DMF (2 mL) was added EDCI (371.05 mg, 1.94 mmol, 3 eq) and HOAt (87.82 mg, 645.19 μmol, 90.25 μL, 1 eq), NMM (326.29 mg, 3.23 mmol, 354.67 μL, 5 eq). The mixture was stirred at 25° C. for 0.5 hr. The mixture was diluted with water (5 mL) and extracted with DCM (5 mL*3). The combined organic layer was dried over Na₂SO₄, filtered and concentrated in vacuo to give a residue. The residue was purified by column chromatography (SiO₂, PE:EA=15:1 to PE:EA=0:1), then the organic liquid was concentrated in vacuo to give a residue. Intermediate 1-3 (220 mg, crude) was obtained as yellow oil. LCMS (Method D): Rt=0.398 min, [M+H]⁺=302.2.

### Step 2: Synthesis of Intermediate 1-4

To a solution of intermediate 1-3 (220 mg, 729.98 μmol, 1 eq) in THF (1 mL) was added LiOH·H₂O (61.26 mg, 1.46 mmol, 2 eq) in MeOH (1 mL) and H₂O (1 mL). The mixture was stirred at 25° C. for 1 hr. The reaction was acidified with 0.5M HCl to pH<7, and extracted with EtOAc (3 mL*3). The combined organic layers were dried over Na₂SO₄, filtered and concentrated under reduced pressure to give a residue. The residue was used in the next step without further purification. Intermediate 1-4 (180 mg, 576.30 μmol, 78.95% yield, 92% purity) was obtained as a white solid. LCMS (Method E): Rt=0.452 min, [M+H]⁺=288.1.

### Step 3: Synthesis of I-888

To a solution of intermediate 1-5 (30 mg, 39.63 μmol, 1 eq) and intermediate 1-4 (13.67 mg, 47.56 μmol, 1.2 eq) in DMF (1 mL) was added EDCI (22.79 mg, 118.90 μmol, 3 eq) NMM (32.07 mg, 317.06 μmol, 34.86 μL, 8 eq) and HOAt (5.39 mg, 39.63 μmol, 5.54 μL, 1 eq). The mixture was stirred at 25° C. for 1 hr. The mixture was concentrated under the pressure to give a residue. The residue was purified by flash silica gel chromatography (ISCO; 40 g SepaFlash Silica Flash Column, Eluent of 0˜60% Ethyl acetate/Petroleum ether gradient @40 mL/min). I-888 (20 mg, 18.65 μmol, 47.06% yield, FA salt) was obtained as a white solid. LCMS (Method E): Rt=0.482 min, [M+H]⁺=1026.6. SFC: Rt=1.589, 1.859 min. 1H NMR (400 MHz, DMSO-d6) δ=12.71-12.38 (m, 1H), 8.69-8.58 (m, 1H), 8.37-8.21 (m, 2H), 8.01-7.93 (m, 1H), 7.91-7.87 (m, 1H), 7.86-7.72 (m, 3H), 7.48-7.34 (m, 4H), 7.29-7.20 (m, 1H), 4.89-4.57 (m, 2H), 4.55-4.12 (m, 6H), 3.63 (s, 2H), 3.58-3.47 (m, 4H), 3.18-3.06 (m, 4H), 2.82-2.74 (m, 2H), 2.71-2.64 (m, 2H), 2.17-1.84 (m, 8H), 1.83-1.54 (m, 10H), 1.53-1.35 (m, 5H), 1.23 (s, 3H), 1.21-0.97 (m, 9H), 0.96-0.74 (m, 3H), 0.62-0.47 (m, 2H). ¹⁹F NMR (377 MHz, DMSO-d6) δ=−119.70-−119.80 (m, 1F).

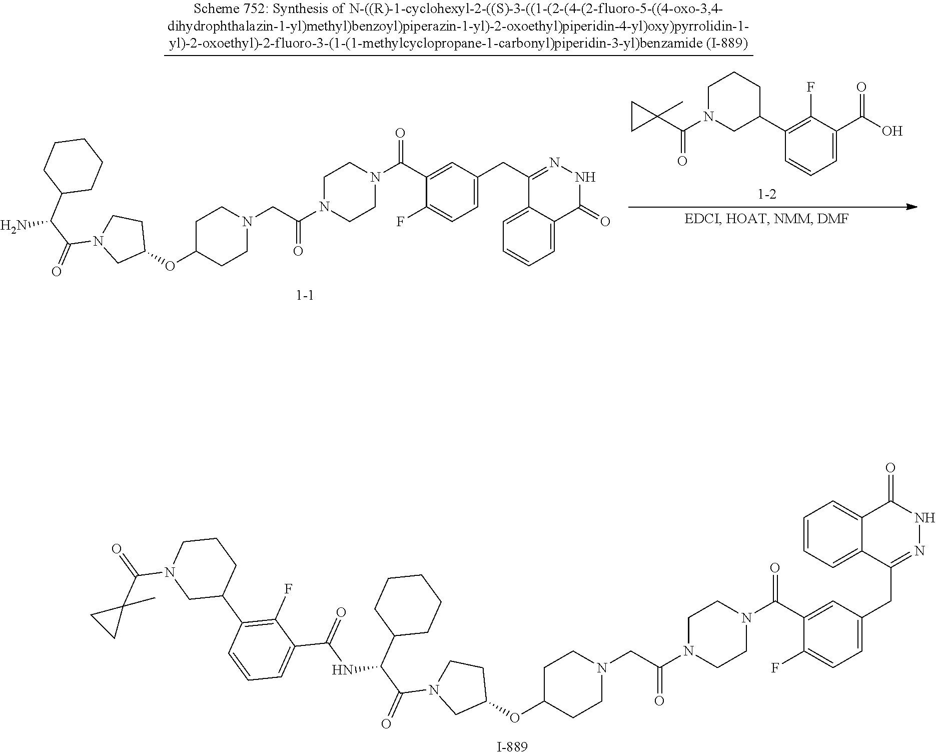 [SMILES: N[C@@H](C(=O)N1CC[C@H](OC2CCN(CC(=O)N3CCN(C(=O)c4cc(Cc5n[nH]c(=O)c6ccccc56)ccc4F)CC3)CC2)C1)C1CCCCC1 ; CC1(C(=O)N2CCCC(c3cccc(C(=O)O)c3F)C2)CC1 ; CC1(C(=O)N2CCCC(c3cccc(C(=O)N[C@@H](C(=O)N4CC[C@H](OC5CCN(CC(=O)N6CCN(C(=O)c7cc(Cc8n[nH]c(=O)c9ccccc89)ccc7F)CC6)CC5)C4)C4CCCCC4)c3F)C2)CC1]

To a solution of Intermediate 1-1 (0.3 g, 398.77 μmol, 1 eq, HCl salt), Intermediate 1-2 (121.76 mg, 398.77 μmol, 1 eq) and HOAt (54.28 mg, 398.77 μmol, 55.78 μL, 1 eq) in DMF (3 mL) was added EDCI (152.89 mg, 797.54 μmol, 2 eq) and NMM (201.68 mg, 1.99 mmol, 219.21 μL, 5 eq). The mixture was stirred at 25° C. for 2 hrs. The mixture was diluted with water (8 mL) and extracted with EtOAc (12 mL*3). The organic layer was concentrated under vacuum to get the crude product. The crude product was purified by reversed-phase HPLC (0.1% NH₃.H₂O condition) and concentrated under vacuum to remove MeCN and dried by lyophilization to give I-889 (350 mg, 348.89 μmol, 87.49% yield) as a white solid. The product was further purified by NP-HPLC (column: Welch Ultimate XB—SiOH 250*50*10 um; mobile phase: [Hexane-EtOH]; gradient: 20%-60% B over 15 min), and concentrated under vacuum to remove solvent, diluted with MeCN (2 mL) and water (50 mL), then lyophilized to give the product. I-889 (211.36 mg, 209.40 μmol, 60.02% yield, 99.39% purity) was obtained as a white solid. LCMS (Method G) Retention time=0.654 min, [M+H]⁺=1003.7. SFC: Retention time=1.710, 2.135 min. ¹H NMR (400 MHz, METHANOL-d4) δ=8.41-8.31 (m, 1H), 7.98-7.91 (m, 1H), 7.91-7.79 (m, 2H), 7.59-7.44 (m, 3H), 7.41-7.34 (m, 1H), 7.26 (br t, J=7.6 Hz, 1H), 7.21-7.13 (m, 1H), 4.87-4.83 (m, 1H), 4.70-4.60 (m, 1H), 4.50 (br d, J=8.6 Hz, 2H), 4.42-4.26 (m, 3H), 4.08-4.00 (m, 1H), 3.83-3.64 (m, 5H), 3.59-3.44 (m, 5H), 3.25 (br d, J=5.6 Hz, 2H), 3.21-3.16 (m, 1H), 3.12-3.02 (m, 1H), 2.81-2.68 (m, 2H), 2.36-2.19 (m, 2H), 2.17-2.07 (m, 1H), 2.06-1.83 (m, 8H), 1.83-1.66 (m, 5H), 1.65-1.48 (m, 3H), 1.38-1.22 (m, 7H), 1.20-1.07 (m, 2H), 1.01-0.82 (m, 3H), 0.69-0.57 (m, 2H). ¹⁹F NMR (377 MHz, METHANOL-d4) δ=−120.75 (br d, J=6.7 Hz, 1F), −121.23-−121.69 (m, 1F).

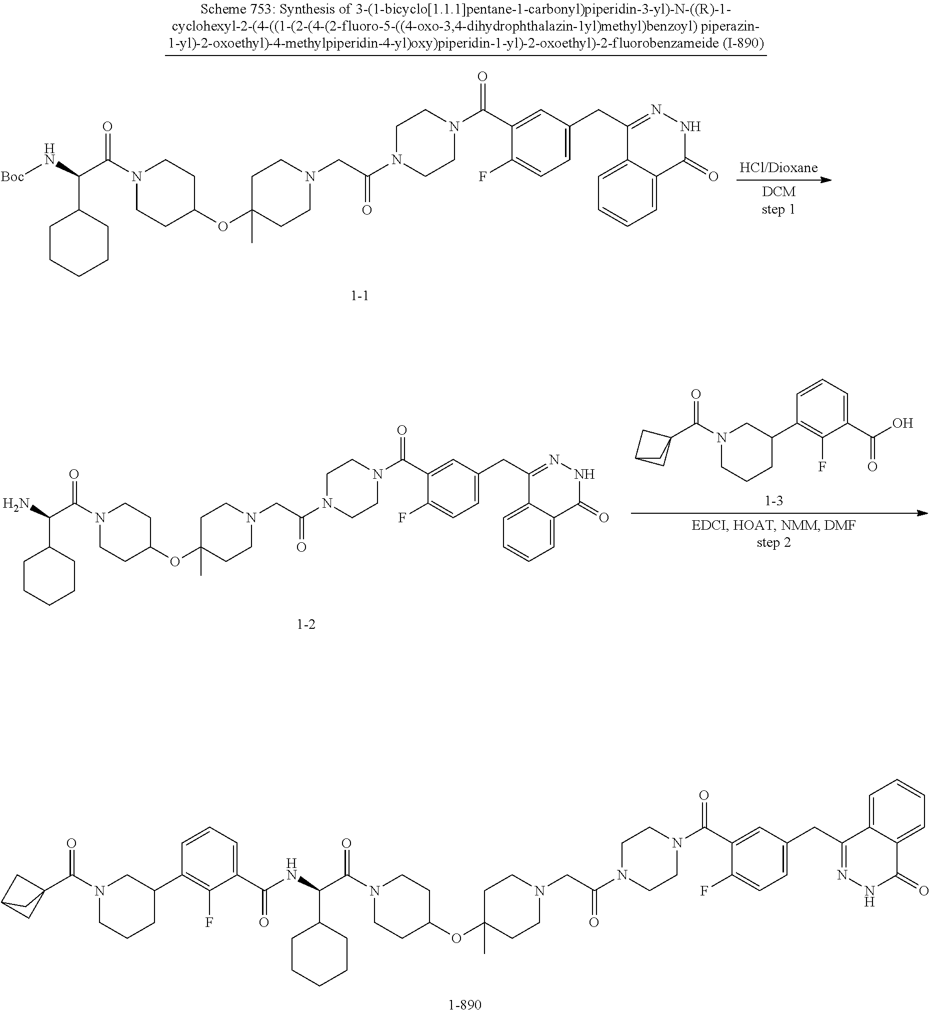 [SMILES: CC(C)(C)OC(=O)N[C@@H](C(=O)N1CCC(OC2(C)CCN(CC(=O)N3CCN(C(=O)c4cc(Cc5n[nH]c(=O)c6ccccc56)ccc4F)CC3)CC2)CC1)C1CCCCC1 ; CC1(OC2CCN(C(=O)[C@H](N)C3CCCCC3)CC2)CCN(CC(=O)N2CCN(C(=O)c3cc(Cc4n[nH]c(=O)c5ccccc45)ccc3F)CC2)CC1 ; O=C(O)c1cccc(C2CCCN(C(=O)C34CC(C3)C4)C2)c1F ; CC1(OC2CCN(C(=O)[C@H](NC(=O)c3cccc(C4CCCN(C(=O)C56CC(C5)C6)C4)c3F)C3CCCCC3)CC2)CCN(CC(=O)N2CCN(C(=O)c3cc(Cc4n[nH]c(=O)c5ccccc45)ccc3F)CC2)CC1]

### Step 1: Synthesis of Intermediate 1-2

To a solution of Intermediate 1-1 (1 g, 1.18 mmol, 1 eq) in DCM (7 mL) was added HCl/Dioxane (4 M, 1 mL). The mixture was stirred at 25° C. for 0.5 ht. The mixture was concentrated under vacuum to give Intermediate 1-2 (0.9 g, crude, HCl salt) as a yellow solid. LCMS (Method G): Retention time=0.561 min [M+H]⁺=744.5.

### Step 2: Synthesis of I-890

To a solution of Intermediate 1-2 (0.4 g, 512.58 μmol, 1 eq, HCl salt) in DMF (4 mL) was added HOAt (69.77 mg, 512.58 μmol, 71.70 μL, 1 eq), EDCI (196.52 mg, 1.03 mmol, 2 eq), NMM (259.23 mg, 2.56 mmol, 281.77 μL, 5 eq) and Intermediate 1-3 (231.46 mg, 512.58 μmol, 1 eq). The mixture was stirred at 25° C. for 1 h. The mixture was poured onto H₂O (10 mL), filtered and the filter cake was washed with H₂O (2 mL*3) and concentrated under vacuum to give a crude product. The crude was twice purified by Prep-HPLC (column: Phenomenex luna C18 150*25 mm*10 um; mobile phase: [water (FA)-ACN]; gradient: 25%-55% B over 15 min). The eluent was concentrated and lyophilized to give I-890 (143.03 mg, 130.76 μmol, 25.51% yield, 99.58% purity, FA salt) as a white solid. LCMS (Method G): Retention time=0.719 min, [M+H]⁺=1043.7. SFC: Retention time=2.339 min, Retention time=3.292 min. ¹H NMR (400 MHz, METHANOL-d4) δ=8.50 (s, 1H), 8.39 (br d, J=7.6 Hz, 1H), 7.97 (br d, J=7.6 Hz, 1H), 7.92-7.82 (m, 2H), 7.63-7.47 (m, 3H), 7.39-7.38 (m, 1H), 7.28-7.27 (m, 1H), 7.22-7.15 (m, 1H), 4.99-4.97 (m, 1H), 4.56 (br d, J=12.4 Hz, 1H), 4.43-4.32 (m, 3H), 4.17-4.05 (m, 1H), 4.03-3.45 (m, 12H), 3.28-2.87 (m, 7H), 2.82-2.67 (m, 1H), 2.54-2.44 (m, 1H), 2.27-2.15 (m, 6H), 2.08-1.50 (m, 19H), 1.37-1.13 (m, 8H). ¹⁹F NMR (377 MHz, METHANOL-d4) δ=−120.71 (br d, J=31.4 Hz, 1F), −121.24-−121.94 (m, 1F).

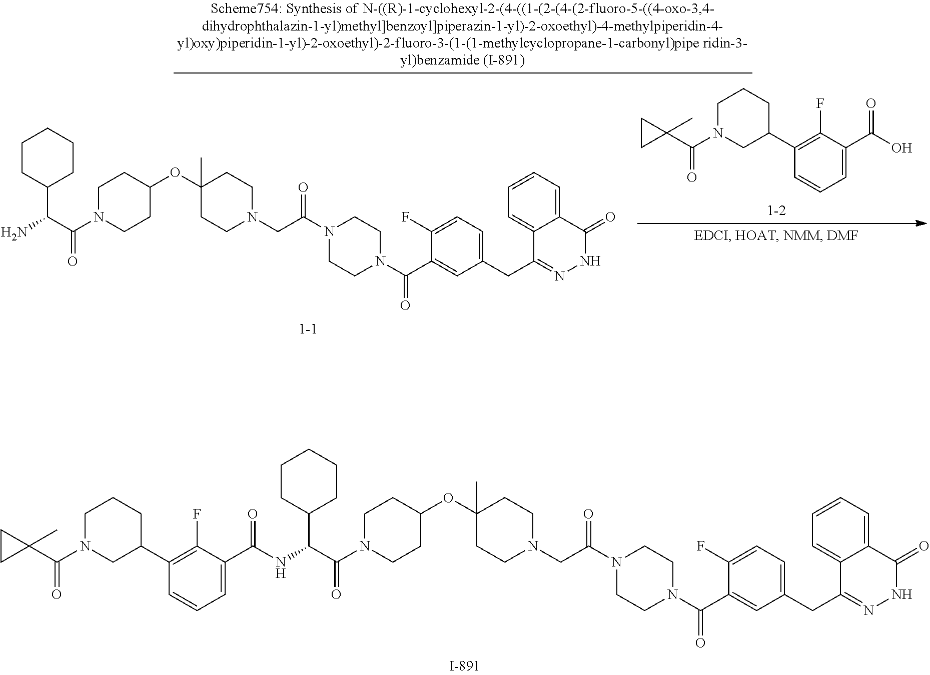 [SMILES: CC1(OC2CCN(C(=O)[C@H](N)C3CCCCC3)CC2)CCN(CC(=O)N2CCN(C(=O)c3cc(Cc4n[nH]c(=O)c5ccccc45)ccc3F)CC2)CC1 ; CC1(C(=O)N2CCCC(c3cccc(C(=O)O)c3F)C2)CC1 ; CC1(OC2CCN(C(=O)[C@H](NC(=O)c3cccc(C4CCCN(C(=O)C5(C)CC5)C4)c3F)C3CCCCC3)CC2)CCN(CC(=O)N2CCN(C(=O)c3cc(Cc4n[nH]c(=O)c5ccccc45)ccc3F)CC2)CC1]

To a solution of Intermediate 1-2 (123.25 mg, 403.66 μmol, 0.9 eq), HOAt (61.05 mg, 448.51 μmol, 62.74 μL, 1 eq), EDCI (171.96 mg, 897.01 μmol, 2 eq), and NMM (226.83 mg, 2.24 mmol, 246.55 L, 5 eq) in DMF (3.5 mL) was added Intermediate 1-1 (350 mg, 448.51 μmol, 1 eq, HCl salt). The mixture was stirred at 25° C. for 1 h. The mixture was poured onto H₂O (15 mL), filtered and the filter cake was washed with H₂O (5 mL*3) and concentrated under vacuum to give a crude product. The crude was purified by Prep-HPLC (column: WatersXbridgePrep OBD C18 150*40 mm*10 um; mobile phase: [water (NH₄HCO₃)-ACN]; gradient: 36%-66% B over 15 min). The eluent was concentrated and lyophilized to give I-891 (169.09 mg, 156.92 μmol, 34.99% yield, 99.97% purity, FA salt) as a white solid. LCMS (Method G): Retention time=0.699 min, [M+H]⁺=1031.7. SFC: Retention time=2.032 min, Retention time=2.638 min. ¹H NMR (400 MHz, METHANOL-d4) δ=8.48 (s, 1H), 8.36 (br d, J=7.6 Hz, 1H), 7.94 (br d, J=7.6 Hz, 1H), 7.90-7.80 (m, 2H), 7.61-7.46 (m, 3H), 7.37-7.36 (m, 1H), 7.30-7.22 (m, 1H), 7.21-7.12 (m, 1H), 4.95 (br d, J=7.6 Hz, 1H), 4.50 (br d, J=12.8 Hz, 2H), 4.38 (s, 2H), 4.15-4.03 (m, 1H), 3.96-3.42 (m, 12H), 3.27-2.87 (m, 7H), 2.04-1.48 (m, 19H), 1.38-1.11 (m, 11H), 0.99-0.87 (m, 2H), 0.62 (br s, 2H). ¹⁹F NMR (377 MHz, METHANOL-d4) δ=−120.71 (br d, J=31.4 Hz, 1F), −121.56 (br d, J=38.1 Hz, 1F).

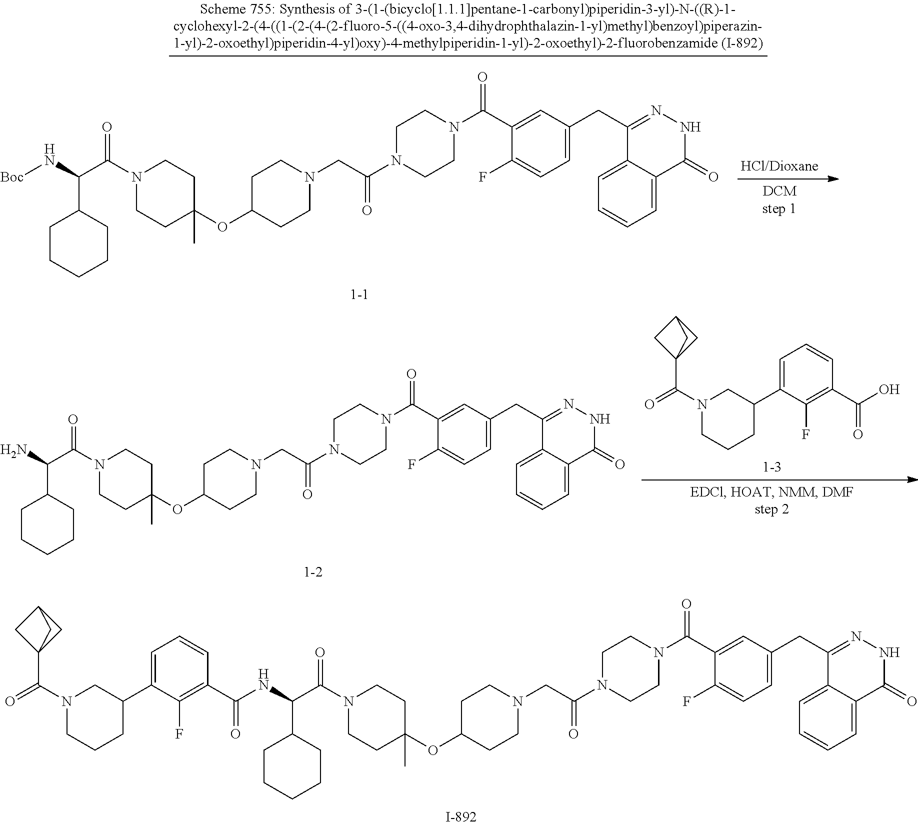 [SMILES: CC(C)(C)OC(=O)N[C@@H](C(=O)N1CCC(C)(OC2CCN(CC(=O)N3CCN(C(=O)c4cc(Cc5n[nH]c(=O)c6ccccc56)ccc4F)CC3)CC2)CC1)C1CCCCC1 ; CC1(OC2CCN(CC(=O)N3CCN(C(=O)c4cc(Cc5n[nH]c(=O)c6ccccc56)ccc4F)CC3)CC2)CCN(C(=O)[C@H](N)C2CCCCC2)CC1 ; O=C(O)c1cccc(C2CCCN(C(=O)C34CC(C3)C4)C2)c1F ; CC1(OC2CCN(CC(=O)N3CCN(C(=O)c4cc(Cc5n[nH]c(=O)c6ccccc56)ccc4F)CC3)CC2)CCN(C(=O)[C@H](NC(=O)c2cccc(C3CCCN(C(=O)C45CC(C4)C5)C3)c2F)C2CCCCC2)CC1]

### Step 1: Synthesis of Intermediate 1-2

To a solution of Intermediate 1-1 (0.85 g, 1.01 mmol, 1 eq) in DCM (7 mL) was added HCl/Dioxane (4 M, 1.06 mL, 4.22 eq). The mixture was stirred at 25° C. for 0.5 h. The mixture was concentrated under vacuum to give Intermediate 1-2 (0.8 g, crude, HCl salt) as a yellow solid. LCMS (Method G): Retention time=0.554 min, [M+H]⁺=744.5.

### Step 2: Synthesis of I-892

To a solution of Intermediate 1-2 (0.4 g, 512.58 μmol, 1 eq, HCl salt) in DMF (4 mL) was added HOAt (69.77 mg, 512.58 μmol, 71.70 μL, 1 eq), EDCI (196.52 mg, 1.03 mmol, 2 eq), NMM (259.23 mg, 2.56 mmol, 281.77 μL, 5 eq) and Intermediate 1-3 (231.46 mg, 512.58 μmol, 1 eq). The mixture was stirred at 25° C. for 1 h. The mixture was poured onto H₂O (10 mL), filtered and the filter cake was washed with H₂O (2 mL*3) and concentrated under vacuum to give a crude product. The crude was purified by Prep-HPLC (column: Welch Ultimate XB—SiOH 250*50*10 um; mobile phase: [Hexane-EtOH]; gradient: 20%-60% B over 15 min) and freeze-dried to give 0.2 g (95.97% purity) of product. Then the product was dissolved in MeOH (2 mL) and re-purified by Prep-HPLC (column: Waters Xbridge 150*25 mm*5 um; mobile phase: [water (NH₄HCO₃)-ACN]; gradient: 38%-68% B over 15 min). The eluent was concentrated and lyophilized to give I-892 (159.94 mg, 152.42 μmol, 79.51% yield, 99.42% purity) as a white solid. LCMS (Method G): Retention time=0.719 min, [M+H]⁺=1043.7. SFC: Retention time=2.447 min, Retention time=3.476 min. ¹H NMR (400 MHz, METHANOL-d4) δ=8.39-8.38 (m, 1H), 7.96 (br d, J=6.8 Hz, 1H), 7.93-7.82 (m, 2H), 7.62-7.46 (m, 3H), 7.40 (br s, 1H), 7.33-7.13 (m, 2H), 5.01-4.94 (m, 1H), 4.55 (br d, J=12.4 Hz, 1H), 4.44-4.31 (m, 3H), 4.26-3.38 (m, 11H), 3.29-3.00 (m, 5H), 2.89-2.67 (m, 3H), 2.53-2.44 (m, 1H), 2.34-2.16 (m, 8H), 2.07-1.42 (m, 19H), 1.37-1.12 (m, 8H). ¹⁹F NMR (377 MHz, METHANOL-d4) δ=−120.72 (br d, J=13.5 Hz, 1F), −121.19-−121.98 (m, 1F).

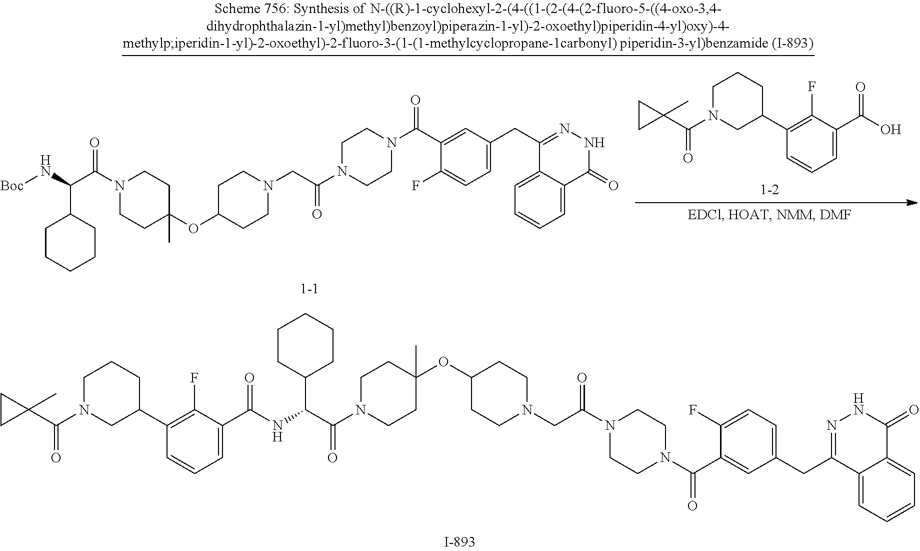 [SMILES: CC(C)(C)OC(=O)N[C@@H](C(=O)N1CCC(C)(OC2CCN(CC(=O)N3CCN(C(=O)c4cc(Cc5n[nH]c(=O)c6ccccc56)ccc4F)CC3)CC2)CC1)C1CCCCC1 ; CC1(C(=O)N2CCCC(c3cccc(C(=O)O)c3F)C2)CC1 ; CC1(OC2CCN(CC(=O)N3CCN(C(=O)c4cc(Cc5n[nH]c(=O)c6ccccc56)ccc4F)CC3)CC2)CCN(C(=O)[C@H](NC(=O)c2cccc(C3CCCN(C(=O)C4(C)CC4)C3)c2F)C2CCCCC2)CC1]

To a solution of Intermediate 1-1 (125.21 mg, 410.06 μmol, 0.8 eq) in DMF (5 mL) was added HOAt (69.77 mg, 512.58 μmol, 71.70 μL, 1 eq), EDCI (196.52 mg, 1.03 mmol, 2 eq), NMM (259.23 mg, 2.56 mmol, 281.77 μL, 5 eq) and Intermediate 1-2 (0.4 g, 512.58 μmol, 1 eq, HCl salt). The mixture was stirred at 25° C. for 1 h. The mixture was poured onto H₂O (15 mL), filtered and the filter cake was washed with H₂O (5 mL*3) and concentrated under vacuum to give a crude product. The crude was purified by Prep-HPLC (column: Waters Xbridge Prep OBD C18 150*40 mm*10 um; mobile phase: [water(NH₄HCO₃)-ACN]; gradient: 36%-66% B over 15 min). The eluent was concentrated and lyophilized to give I-893 (188.42 mg, 181.31 μmol, 35.37% yield, 99.23% purity) as a white solid. LCMS (Method G): Retention time=0.694 min, [M+H]⁺=1031.6. SFC: Retention time=3.319 min, Retention time=4.553 min. ¹H NMR (400 MHz, METHANOL-d4) δ=8.39-8.37 (m, 1H), 8.02-7.94 (m, 1H), 7.93-7.82 (m, 2H), 7.62-7.48 (m, 3H), 7.40 (br d, J=5.2 Hz, 1H), 7.29-7.28 (m, 1H), 7.22-7.14 (m, 1H), 5.01-4.94 (m, 2H), 4.52 (br d, J=9.2 Hz, 2H), 4.40 (br d, J=2.4 Hz, 2H), 4.28-3.91 (m, 2H), 3.90-3.45 (m, 9H), 3.28-3.07 (m, 4H), 2.81 (br d, J=5.2 Hz, 2H), 2.30 (br d, J=8.4 Hz, 2H), 2.06-1.44 (m, 19H), 1.43-1.03 (m, 12H), 1.01-0.87 (m, 2H), 0.65 (br s, 2H). ¹⁹F NMR (376 MHz, METHANOL-d4) δ=−120.74 (br d, J=11.2 Hz, 1F), −121.37-−121.82 (m, 1F).

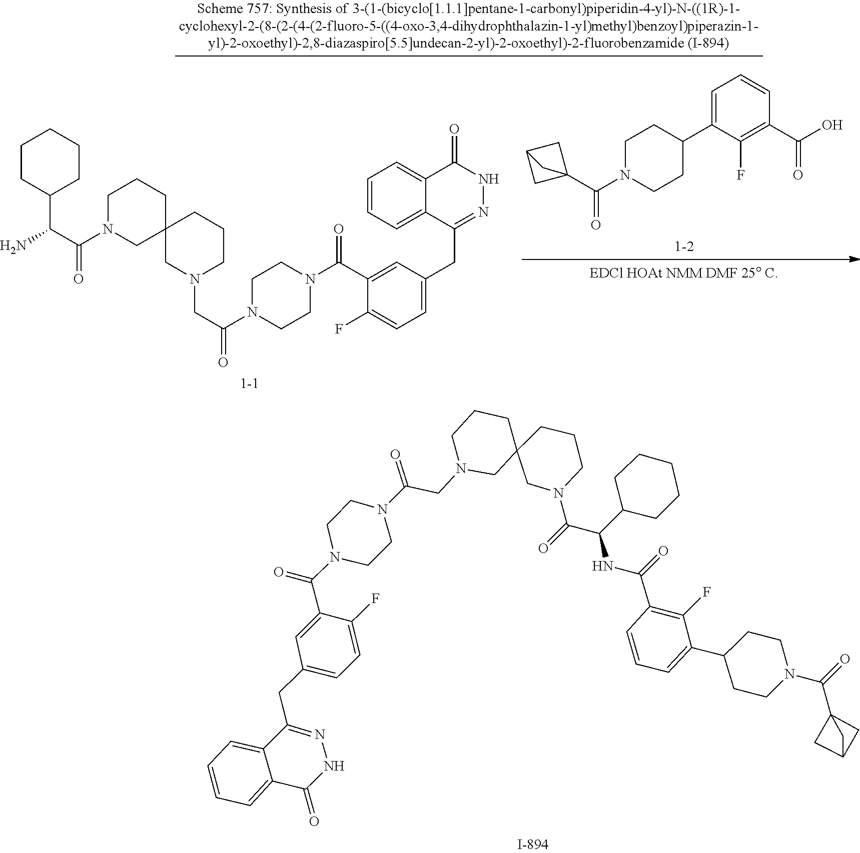 [SMILES: N[C@@H](C(=O)N1CCCC2(CCCN(CC(=O)N3CCN(C(=O)c4cc(Cc5n[nH]c(=O)c6ccccc56)ccc4F)CC3)C2)C1)C1CCCCC1 ; O=C(O)c1cccc(C2CCN(C(=O)C34CC(C3)C4)CC2)c1F ; O=C(N[C@@H](C(=O)N1CCCC2(CCCN(CC(=O)N3CCN(C(=O)c4cc(Cc5n[nH]c(=O)c6ccccc56)ccc4F)CC3)C2)C1)C1CCCCC1)c1cccc(C2CCN(C(=O)C34CC(C3)C4)CC2)c1F]

To a solution of intermediate 1-1 (50 mg, 67.91 μmol, 1 eq, HCl salt) and intermediate 1-2 (25.86 mg, 81.49 μmol, 1.2 eq) in DMF (0.5 mL) was added EDCI (39.05 mg, 203.72 μmol, 3 eq) and HOAt (9.24 mg, 67.91 μmol, 9.50 μL, 1 eq), and NMM (54.95 mg, 543.24 μmol, 59.73 μL, 8 eq). The mixture was stirred at 25° C. for 1 hr. The reaction mixture was quenched by addition of water (5 mL) at 25° C., and then extracted with EA (5 mL*3). The combined organic layers were dried over Na₂SO₄, filtered and concentrated under reduced pressure to give a residue. The residue was purified by prep-HPLC (column: Phenomenex luna C18 150*25 mm*10 um; mobile phase: [water (FA)-ACN]; gradient: 25%-55% B over 15 min), and the target peak was concentrated and lyophilized to afford I-894 (25 mg, 23.82 μmol, 35.08% yield, 99.59% purity, FA salt) as a white solid. LCMS (Method E): Rt=0.509 min, [M+H]⁺=999.5. SFC: Rt=0.712 min. ¹H NMR (400 MHz, METHANOL-d4) δ=8.37 (s, 1H), 8.35 (d, J=2.8 Hz, 1H), 7.94 (d, J=6.4 Hz, 1H), 7.89-7.79 (m, 2H), 7.59-7.32 (m, 4H), 7.27-7.07 (m, 2H), 5.01-4.89 (m, 1H), 4.59 (d, J=12.0 Hz, 1H), 4.44-4.30 (m, 3H), 3.94-3.67 (m, 4H), 3.64-3.34 (m, 6H), 3.27-3.12 (m, 4H), 2.78-2.66 (m, 2H), 2.49-2.44 (m, 1H), 2.27-2.11 (m, 7H), 1.93-1.50 (m, 18H), 1.43 (d, J=7.2 Hz, 2H), 1.33-1.07 (m, 6H), 0.96 (m, 1H). ¹⁹F NMR (377 MHz, METHANOL-d4) δ=−120.17-−120.74 (m, 1F), −121.84-122.53 (m, 1F).

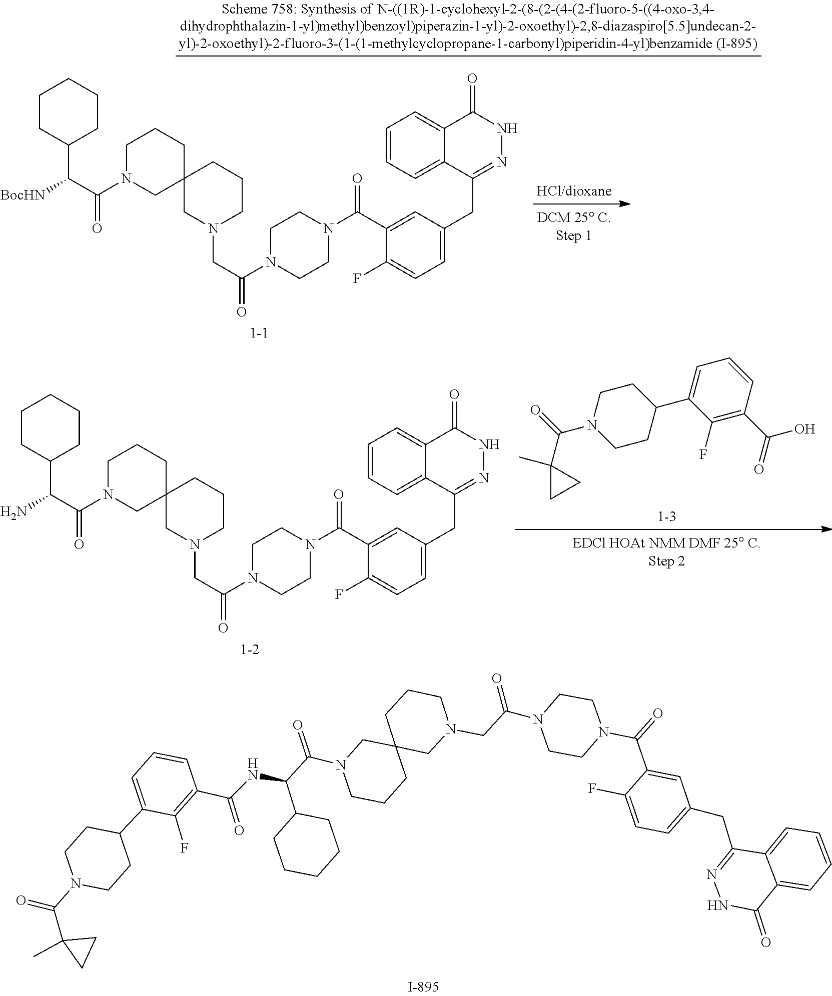

### Step 1: Synthesis of Intermediate 1-2

To a solution of Intermediate 1-1 (50 mg, 62.50 μmol, 1 eq) in DCM (0.5 mL) was added HCl/dioxane (2 M, 0.5 mL, 16.00 eq). The mixture was stirred at 25° C. for 1 hr. The reaction mixture was concentrated under reduced pressure to give Intermediate 1-2 (50 mg, crude, HCl salt) as white solid which was used in the next step without further purification. LCMS: Rt=0.395 min, [M+H]⁺=700.4.

### Step 2: Synthesis of I-895

To a solution of Intermediate 1-2 (45 mg, 61.12 μmol, 1 eq, HCl salt) and Intermediate 1-3 (18.66 mg, 61.12 μmol, 1 eq) in DMF (0.5 mL) was added EDCI (35.15 mg, 183.35 μmol, 3 eq) and HOAt (8.32 mg, 61.12 μmol, 8.55 μL, 1 eq), NMM (49.45 mg, 488.92 μmol, 53.75 μL, 8 eq). The mixture was stirred at 25° C. for 1 hr. The reaction mixture was quenched by addition of water (5 mL) at 25° C., and then extracted with EA (5 mL*3). The combined organic layers were dried over Na₂SO₄, filtered and concentrated under reduced pressure to give a residue. The residue was purified by prep-HPLC (column: Phenomenex luna C18 150*25 mm*10 um; mobile phase: [water (FA)-ACN]; gradient: 20%-50% B over 15 min), and the eluent was concentrated and lyophilized to afford 1-895 (27 mg, 25.74 μmol, 42.12% yield, 98.5% purity, FA salt) as a white solid. LCMS (Method E): Rt=0.512 min, [M+H]⁺=987.6. SFC: Rt=2.267 min, ee %>99%. ¹H NMR (400 MHz, METHANOL-d4) δ=8.52-8.44 (m, 1H), 8.41-8.28 (m, 1H), 8.00-7.91 (m, 1H), 7.90-7.78 (m, 2H), 7.62-7.29 (m, 4H), 7.28-7.04 (m, 2H), 5.14-4.91 (m, 1H), 4.65-4.46 (m, 2H), 4.43-4.25 (m, 2H), 4.04-3.38 (m, 10H), 3.29-2.83 (m, 6H), 2.82-2.30 (m, 3H), 2.26-2.07 (m, 1H), 2.05-1.39 (m, 18H), 1.35-1.06 (m, 9H), 0.96-0.84 (m, 2H), 0.68-0.56 (m, 2H). ¹⁹F NMR (377 MHz, METHANOL-d4) δ=−120.28-−120.80 (m, 1F), −121.77-−122.55 (m, 1F).

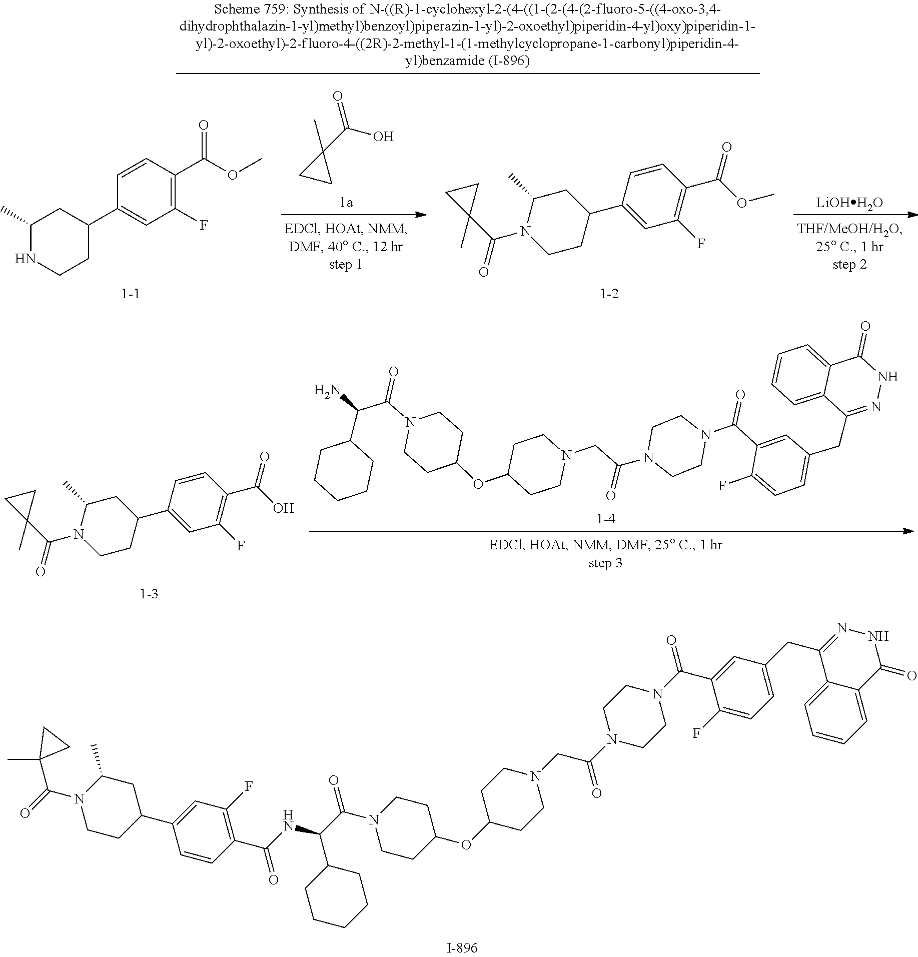

### Step 1: Synthesis of Intermediate 1-2

To a solution of 1-methylcyclopropanecarboxylic acid (62.63 mg, 625.53 μmol, 1.2 eq) in DMF (2 mL) was added EDCI (299.79 mg, 1.56 mmol, 3 eq), HOAt (70.95 mg, 521.27 μmol, 72.92 μL, 1 eq) and NMM (263.63 mg, 2.61 mmol, 286.55 μL, 5 eq). Then intermediate 1-1 (150 mg, 521.27 μmol, 1 eq, HCl salt) was added. The mixture was stirred at 40° C. for 12 hrs. The mixture was washed with water (4 mL) and extracted with EA (5 mL*3). The combined organic layers were dried over sodium sulfate, filtered and concentrated under reduced pressure to give a residue. The residue was purified by flash silica gel chromatography (ISCO; 12 g SepaFlash Silica Flash Column, Eluent of 0˜100% EA/PE @25 mL/min) and then the eluent was concentrated in vacuo to give intermediate 1-2 (170 mg, 499.71 μmol, 95.86% yield, 98% purity) as a yellow gum. LCMS (Method D): Rt=0.379 min, [M+H]⁺=334.5. ¹H NMR (400 MHz, METHANOL-d4) δ=7.87-7.85 (m, 1H), 7.22-7.12 (m, 2H), 4.29-4.27 (m, 1H), 4.21-4.19 (m, 1H), 3.89 (s, 3H), 3.55-3.42 (m, 1H), 2.87-2.76 (m, 1H), 2.35-2.21 (m, 1H), 2.02-1.93 (m, 1H), 1.82-1.81 (m, 1H), 1.80-1.73 (m, 1H), 1.33 (s, 3H), 1.28-1.14 (m, 3H), 1.00-0.94 (m, 1H), 0.90-0.84 (m, 1H), 0.66-0.62 (m, 2H). ¹⁹FNMR (376 MHz, METHANOL-d4) δ=−111.412. SFC: SFC: Rt=1.538 min, 1.783 min.

### Step 2: Synthesis of Intermediate 1-3

To a solution of intermediate 1-2 (150 mg, 449.92 μmol, 1 eq) in THF (0.5 mL), MeOH (0.5 mL) and H₂O (0.5 mL) was added LiOH·H₂O (56.64 mg, 1.35 mmol, 3 eq). The mixture was stirred at 25° C. for 1 hr. The pH was acidified to 5-6 with 1N aq·HCl, and the mixture was washed with water (5 mL) and extracted with EA (8 mL*3). The combined organic layers were dried over sodium sulfate, filtered and concentrated under reduced pressure to give intermediate 1-3 (140 mg, 420.83 μmol, 93.54% yield, 96% purity) as a yellow solid. LCMS (Method D): Rt=0.376 min, [M+H]⁺=320.2. SFC: Rt=2.175 min, 2.392 min. ¹HNMR (400 MHz, METHANOL-d4) δ=7.88-7.85 (m, 1H), 7.21-7.11 (m, 2H), 4.29-4.27 (m, 1H), 4.21-4.19 (m, 1H), 3.48-3.47 (m, 1H), 2.87-2.75 (m, 1H), 2.35-2.22 (m, 1H), 1.99-1.94 (m, 1H), 1.88-1.82 (m, 1H), 1.79-1.78 (m, 1H), 1.33 (s, 3H), 1.24-1.15 (m, 3H), 1.01-0.95 (m, 1H), 0.90-0.84 (m, 1H), 0.65-0.61 (m, 2H). ¹⁹FNMR (376 MHz, METHANOL-d4) δ=−111.524.

### Step 3: Synthesis of I-896

To a solution of intermediate 1-3 (62.51 mg, 195.74 μmol, 1 eq) in DMF (1.5 mL) was added EDCI (112.57 mg, 587.21 μmol, 3 eq), HOAt (26.64 mg, 195.74 μmol, 27.38 μL, 1 eq) and NMM (98.99 mg, 978.68 μmol, 107.60 μL, 5 eq). Then intermediate 1-4 (150 mg, 195.74 μmol, 1 eq, HCl salt) was added. The mixture was stirred at 25° C. for 1 hr. The mixture was washed with water (3 mL) and extracted with EA (5 mL*3). The combined organic layers dried over sodium sulfate, filtered and concentrated under reduced pressure to give a residue. The residue was purified by prep-HPLC (column: Phenomenex luna C18 150*25 mm*10 um; mobile phase: [water(FA)-ACN]; gradient: 28%-588% B over 1 min), and the eluent was concentrated to remove organic solvents and lyophilized to give I-896 (58.45 mg, 54.26 μmol, 27.98% yield, 100% purity, FA salt) as a white solid. LCMS (Method E): Rt: 0.513 min, [M+H]⁺=1031.7. ¹HNMR (400 MHz, METHANOL-d4) δ=8.46 (s, 1H), 8.37 (br d, J=7.6 Hz, 1H), 7.99-7.92 (m, 1H), 7.91-7.80 (m, 2H), 7.71 (m, 1H), 7.50 (br s, 1H), 7.37 (m, 1H), 7.26-7.12 (m, 3H), 4.98 (br d, J=6.8 Hz, 1H), 4.39 (s, 2H), 4.34-4.15 (m, 2H), 4.09-3.95 (m, 1H), 3.94-3.41 (m, 14H), 3.40-3.33 (m, 2H), 3.10-2.90 (m, 2H), 2.88-2.54 (m, 3H), 2.37-2.20 (m, 1H), 2.06-1.39 (m, 19H), 1.37-1.22 (m, 6H), 1.21-1.04 (m, 5H), 1.02-0.80 (m, 2H), 0.69-0.59 (m, 2H).

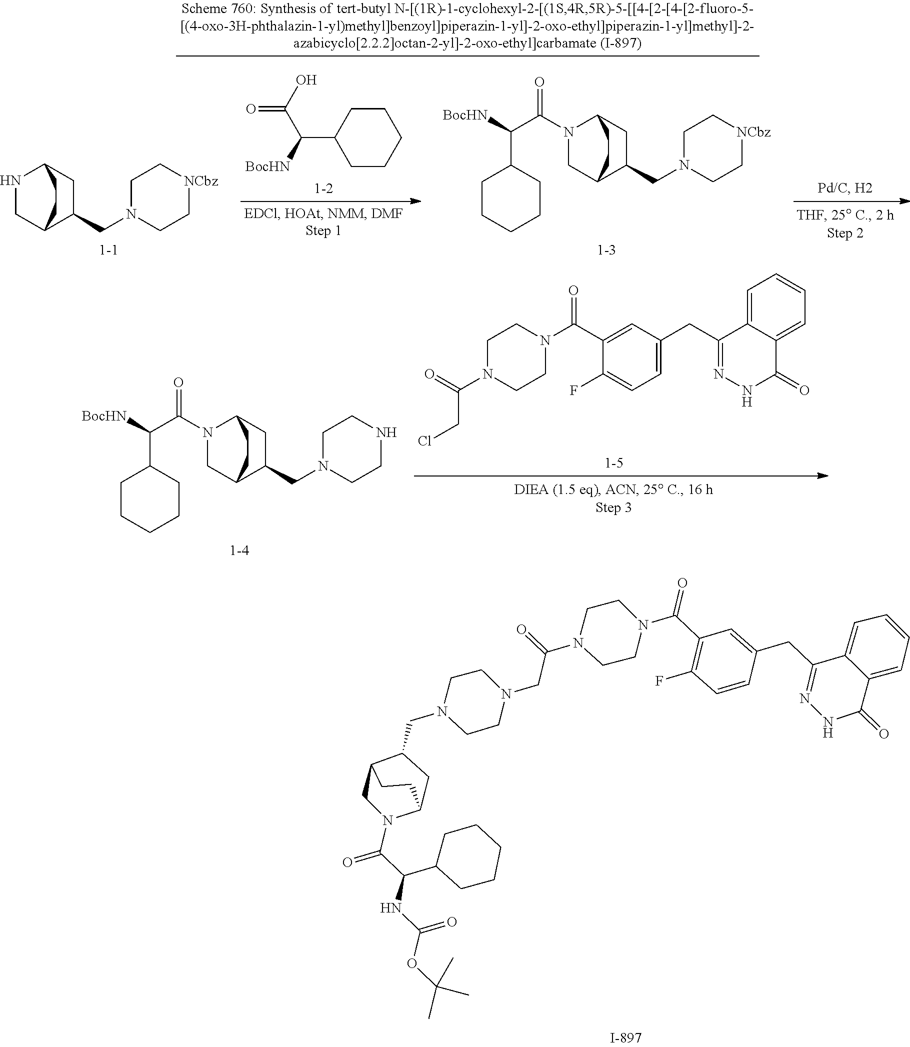 [SMILES: O=C(OCc1ccccc1)N1CCN(C[C@@H]2C[C@@H]3CC[C@H]2CN3)CC1 ; CC(C)(C)OC(=O)N[C@@H](C(=O)O)C1CCCCC1 ; CC(C)(C)OC(=O)N[C@@H](C(=O)N1C[C@@H]2CC[C@H]1C[C@H]2CN1CCN(C(=O)OCc2ccccc2)CC1)C1CCCCC1 ; CC(C)(C)OC(=O)N[C@@H](C(=O)N1C[C@@H]2CC[C@H]1C[C@H]2CN1CCNCC1)C1CCCCC1 ; O=C(CCl)N1CCN(C(=O)c2cc(Cc3n[nH]c(=O)c4ccccc34)ccc2F)CC1 ; CC(C)(C)OC(=O)N[C@@H](C(=O)N1C[C@@H]2CC[C@H]1C[C@H]2CN1CCN(CC(=O)N2CCN(C(=O)c3cc(Cc4n[nH]c(=O)c5ccccc45)ccc3F)CC2)CC1)C1CCCCC1]

### Step 1: Synthesis of Intermediate 1-3

To a solution of Intermediate 1-2 (67.73 mg, 263.21 μmol, 1 eq) in DMF (1 mL) was added HOAt (53.74 mg, 394.82 μmol, 55.23 μL, 1.5 eq) and EDCI (151.37 mg, 789.63 μmol, 3 eq), NMM (133.12 mg, 1.32 mmol, 144.69 μL, 5 eq), Intermediate 1-1 (100.00 mg, 263.21 μmol, 1 eq, HCl salt). The resulting mixture was stirred at 25° C. for 1 hr. The reaction mixture was quenched by water (4 mL), and extracted by EA (2 mL*3). The organic layers were washed with brine (5 mL*2). The organic phase was combined and concentrated to afford the crude. The crude product was purified by reverse phase chromatography (20 g of XB—C18, 20-40 m, 120 Å) Mobile phase: A for H₂O (0.1% FAv/v) and B for acetonitrile; Gradient: B5%-100% in 30 min; Flow rate: 100 ml/min; Column temperature: R.T. Wavelength: 220 nm/254 nm), and the eluent concentrated to remove organic solvents and lyophilized to give Intermediate 1-3 (60 mg, 102.96 μmol, 39.12% yield) as a yellow solid. LCMS (Method E): Rt=0.484 min, [M+H]⁺=583.8. SFC: Rt=1.334 min, 1.432 min. ¹H NMR (400 MHz, CHLOROFORM-d) δ=7.41-7.29 (m, 5H), 5.31-5.18 (m, 1H), 5.14 (s, 2H), 4.53-4.22 (m, 1H), 3.94 (br s, 1H), 3.80-3.29 (m, 6H), 2.52-2.19 (m, 6H), 2.04-1.94 (m, 3H), 1.87-1.63 (m, 8H), 1.43 (s, 9H), 1.32-0.83 (m, 9H).

### Step 2: Synthesis of Intermediate 1-4

To a solution of intermediate 1-3 (60.00 mg, 102.96 μmol, 1 eq) in MeOH (1 mL) was added Pd/C (54.78 mg, 51.48 μmol, 10% purity, 0.5 eq) under N₂, the reaction mixture was purged with H₂ three times and the reaction mixture was stirred at 25° C. for 2 h under H₂ (15 psi). The suspension was filtered through a pad of Celite and the filter cake was washed with MeOH (2 mL*5). The combined filtrates were concentrated to dryness to give intermediate 1-4 (35 mg, 78.01 μmol, 75.77% yield) as a colorless oil. LCMS (Method E): Rt=0.415 min, [M+H]⁺=449.3.

### Step 4: Synthesis of I-897

To Intermediate 1-4 (35 mg, 78.01 μmol, 1 eq) in MeCN (0.5 mL) was added Intermediate 1-5 (34.55 mg, 78.01 μmol, 1 eq) and DIEA (15.12 mg, 117.02 μmol, 20.38 μL, 1.5 eq), and the reaction mixture was stirred at 25° C. for 16 h. The reaction mixture was quenched by water (4 mL), and extracted by EA (2 mL*3). The organics were washed by brine (5 mL*2). The organic phase was dried by anhydrous Na₂SO₄, filtered and the concentrated to afford the crude. The crude product was purified by reverse phase chromatography (20 g of XB—C18, 20-40 μm, 120 Å) Mobile phase: A for H₂O (0.1% NH₃·H₂O v/v) and B for acetonitrile; Gradient: B5%-100% in 30 min; Flow rate: 100 ml/min; Column temperature: R.T. Wavelength: 220 nm/254 nm), and the eluent was concentrated to remove organic solvents and lyophilized to give I-897 (38 mg, 44.44 μmol, 56.97% yield, 100% purity) as a white solid. LCMS (Method G): Rt=0.664 min, [M+H]⁺=855.6. SFC: Rt=1.182 min, 1.739 min. ¹H NMR (400 MHz, DMSO-d6) δ=12.60 (s, 1H), 8.26 (d, J=7.6 Hz, 1H), 8.01-7.94 (m, 1H), 7.93-7.78 (m, 2H), 7.48-7.34 (m, 2H), 7.24 (t, J=8.4 Hz, 1H), 6.83-6.72 (m, 1H), 4.37-4.22 (m, 2H), 4.10-3.73 (m, 2H), 3.66-3.43 (m, 5H), 3.36 (d, J=4.4 Hz, 2H), 3.22-3.08 (m, 4H), 2.18 (br s, 10H), 1.98-1.78 (m, 3H), 1.74-1.46 (m, 9H), 1.35 (s, 9H), 1.11 (d, J=4.4 Hz, 4H), 1.03-0.87 (m, 4H). ¹⁹F NMR (377 MHz, DMSO-d6) δ=−119.79 (s, 1F).

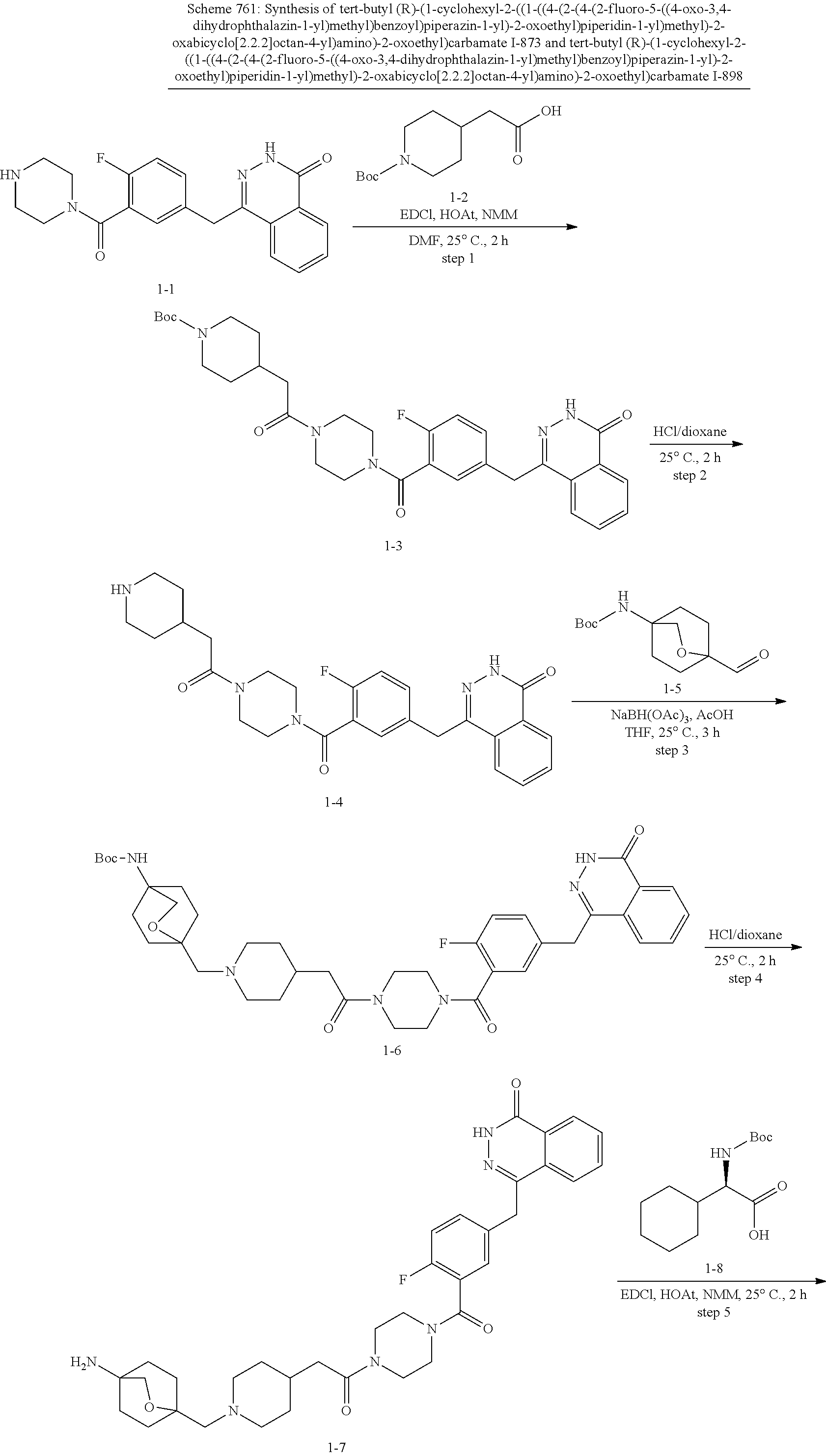

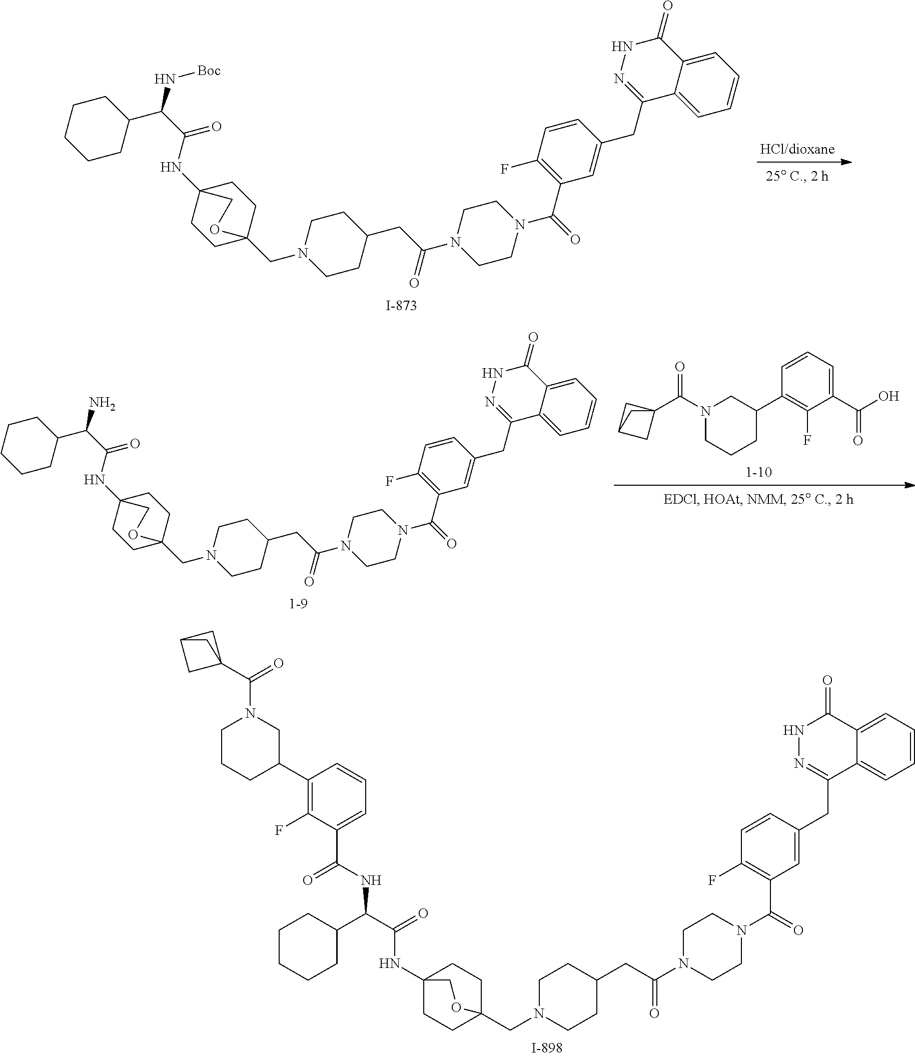

### Step 1: Synthesis of Intermediate 1-3

To a solution of Intermediate 1-2 (438.27 mg, 1.80 mmol, 1.1 eq) in DMF (12 mL) was added HOAt (445.79 mg, 3.28 mmol, 458.16 μL, 2 eq) and EDCI (627.86 mg, 3.28 mmol, 2 eq). The mixture was stirred at 25° C. for 30 min. Then Intermediate 1-1 (600 mg, 1.64 mmol, 1 eq) and NMM (496.92 mg, 4.91 mmol, 540.13 μL, 3 eq) were added and the mixture was stirred at 25° C. for 2 hr. The mixture was poured into 20 mL of water and extracted with EA (20 mL*2). The combined organic phase was dried over anhydrous Na₂SO₄, filtered and concentrated in vacuum. The crude product was purified by reversed-phase HPLC (0.1% FA condition). The eluent was concentrated and lyophilized to give Intermediate 1-3 (900 mg, 1.52 mmol, 92.89% yield) as a yellow solid. LCMS (Method E): Rt=0.515 min, [M+H]⁺=592.4. ¹H NMR (400 MHz, CHLOROFORM-d) δ=10.36-10.03 (m, 1H), 8.54-8.42 (m, 1H), 7.84-7.66 (m, 3H), 7.40-7.30 (m, 2H), 7.06 (br t, J=8.6 Hz, 1H), 4.29 (s, 2H), 4.12-4.03 (m, 2H), 3.83-3.70 (m, 2H), 3.57 (br d, J=4.0 Hz, 2H), 3.45 (br s, 1H), 3.38-3.19 (m, 2H), 2.81-2.65 (m, 2H), 2.33-2.19 (m, 2H), 2.04-1.95 (m, 1H), 1.74 (br d, J=10.9 Hz, 2H), 1.62 (br s, 2H), 1.46 (s, 9H), 1.20-1.06 (m, 2H).

### Step 2: Synthesis of Intermediate 1-4

A mixture of Intermediate 1-3 (900 mg, 1.52 mmol, 1 eq) and HCl/dioxane (2 M, 21.43 mL, 28.17 eq) was stirred at 25° C. for 2 hr. The mixture was concentrated in vacuum and dissolved in 10 mL of water, basified to pH=10 with aq·Na₂CO₃, and extracted with DCM (50 mL*2). The combined organic phase was dried over anhydrous Na₂SO₄, filtered and concentrated in vacuum. The residue was used directly without further purification. Intermediate 1-4 (700 mg, 1.42 mmol, 93.62% yield) was obtained as a yellow solid. LCMS (Method E): Rt=0.385 min, [M+H]⁺=492.2.

### Step 3: Synthesis of Intermediate 1-6

To a solution of Intermediate 1-5 (363.57 mg, 1.42 mmol, 1 eq) and Intermediate 1-4 (700 mg, 1.42 mmol, 1 eq) in DCM (14 mL) was added AcOH (855.17 g, 14.24 μmol, 8.15e−1 μL, 0.01 eq). The mixture was stirred at 25° C. for 1 hr. Then sodium;triacetoxyboranuide (905.44 mg, 4.27 mmol, 3 eq) was added and the mixture was stirred at 25° C. for 1 hr. The mixture was poured into 20 mL of aq·NH₄Cl and extracted with DCM (20 mL*2). The combined organic phase was dried over anhydrous Na₂SO₄, filtered and concentrated in vacuum. The crude product was purified by reversed-phase HPLC (0.10% FA condition). The eluent was concentrated and lyophilized to give Intermediate 1-6 (650 mg, 889.36 μmol, 62.45% yield) as a yellow gum. LCMS (Method E): Rt=0.477 min, [M+H]⁺=731.4. ¹H NMR (400 MHz, CHLOROFORM-d) δ=10.35-10.18 (m, 1H), 10.03 (s, 1H), 8.50-8.43 (m, 2H), 7.84-7.75 (m, 2H), 7.75-7.66 (m, 1H), 7.37-7.31 (m, 2H), 7.06 (t, J=9.1 Hz, 1H), 4.29 (s, 3H), 3.91 (s, 2H), 3.86-3.62 (m, 3H), 3.57 (br d, J=4.5 Hz, 2H), 3.47-3.22 (m, 5H), 2.63-2.55 (m, 2H), 2.44-2.20 (m, 12H), 2.14-1.92 (m, 6H), 1.87-1.69 (m, 7H), 1.66-1.50 (m, 2H), 1.42 (s, 9H). ¹⁹F NMR (377 MHz, METHANOL-d₄) δ=−117.55 (br d, J=13.5 Hz, 1F).

### Step 4: Synthesis of Intermediate 1-7

A mixture of Intermediate 1-6 (650 mg, 889.36 μmol, 1 eq) and HCl/dioxane (2 M, 18.57 mL, 41.76 eq) was stirred at 25° C. for 2 hr. The mixture was concentrated in vacuum. The residue was used directly without further purification. Intermediate 1-7 (560 mg, crude, HCl salt) was obtained as a white solid. LCMS (Method E): Rt=0.377 min, [M+H]⁺=631.4. ¹H NMR (400 MHz, CHLOROFORM-d) δ=8.37 (d, J=7.3 Hz, 1H), 8.09 (s, 1H), 7.99-7.92 (m, 1H), 7.92-7.79 (m, 2H), 7.55-7.46 (m, 1H), 7.40-7.31 (m, 1H), 7.17 (t, J=8.9 Hz, 1H), 4.39 (s, 2H), 3.94 (s, 2H), 3.55-3.43 (m, 3H), 3.21 (s, 2H), 3.15-3.03 (m, 2H), 2.56-2.34 (m, 2H), 2.27-2.11 (m, 2H), 2.10-1.87 (m, 9H), 1.77-1.64 (m, 2H). ¹⁹F NMR (377 MHz, METHANOL-d₄) δ=−120.77 (br d, J=29.2 Hz, 1F).

### Step 5: Synthesis of I-873

To a solution of Intermediate 1-8 (385.67 mg, 1.50 mmol, 2 eq) in DMF (10 mL) was added HOAt (306.00 mg, 2.25 mmol, 314.49 μL, 3 eq) and EDCI (430.98 mg, 2.25 mmol, 3 eq). The mixture was stirred at 25° C. for 30 min. Then Intermediate 1-7 (500 mg, 749.39 μmol, 1 eq, HCl salt) and NMM (1.14 g, 11.24 mmol, 1.24 mL, 15 eq) was added and the mixture was stirred at 25° C. for 2 hr. The mixture was poured into 10 mL of water and extracted with EA (10 mL*2). The combined organic phase was dried over anhydrous Na₂SO₄, filtered and concentrated in vacuum. The crude product was purified by reversed-phase HPLC (0.1% NH₃·H₂O). The eluent was concentrated and lyophilized to give I-873 (300 mg, 327.24 μmol, 43.67% yield, 99.926% purity, FA salt) as a yellow solid. LCMS (Method E): Rt=0.695 min, [M+H]⁺=870.6. ¹H NMR (400 MHz, CHLOROFORM-d) δ=10.31-10.09 (m, 1H), 8.52-8.42 (m, 1H), 7.89-7.65 (m, 3H), 7.40-7.30 (m, 2H), 7.06 (br t, J=8.3 Hz, 1H), 5.66-5.45 (m, 1H), 5.31 (s, 4H), 5.15-4.94 (m, 1H), 4.29 (s, 2H), 4.05-3.92 (m, 2H), 3.86-3.54 (m, 6H), 3.50-3.38 (m, 1H), 3.36-3.20 (m, 2H), 2.99-2.74 (m, 2H), 2.34-2.19 (m, 4H), 2.12 (br d, J=10.6 Hz, 4H), 2.02-1.93 (m, 1H), 1.92-1.83 (m, 2H), 1.44 (s, 9H). ¹⁹F NMR (377 MHz, METHANOL-d₄) δ=−117.55 (br d, J=11.2 Hz, 1F).

### Step 5: Synthesis of Intermediate 1-9

A mixture of I-873 (300 mg, 344.80 μmol, 1 eq) and HCl/dioxane (2 M, 10 mL, 58.00 eq) was stirred at 25° C. for 1 hr. The mixture was concentrated in vacuum. The residue was used directly without further purification. Intermediate 1-9 (280 mg, crude, HCl salt) was obtained as a gray solid. LCMS (Method E): Rt=0.395 min, [M+H]⁺=770.4.

### Step 6: Synthesis of I-898

To a solution of Intermediate 1-10 (20.46 mg, 64.48 μmol, 1.3 eq) in DMF (1 mL) was added HOAt (13.50 mg, 99.21 μmol, 13.88 μL, 2 eq) and EDCI (19.02 mg, 99.21 μmol, 2 eq). The mixture was stirred at 25° C. for 30 min. Then Intermediate 1-9 (40 mg, 49.60 μmol, 1 eq, HCl salt) and NMM (25.09 mg, 248.01 μmol, 27.27 μL, 5 eq) were added and the mixture was stirred at 25° C. for 1 hr. The mixture was poured into 10 mL of water and extracted with EA (10 mL*2). The combined organic phase was dried over anhydrous Na₂SO₄, filtered and concentrated in vacuum. The residue was purified by reversed phase chromatography (26.8*125 mm, 80 g of XB—C18, 20-40 μm, 120 Å) Mobile phase: A for H₂O (0.1% NH₃·H₂O) and B for acetonitrile; Gradient: B 0%-100% in 15 min; Flow rate: 40 mL/min; Column temperature: R.T. Wavelength: 220 nm/254 nm), and he target peak was concentrated and lyophilized to afford I-898 (12.77 mg, 10.79 μmol, 23.08% yield, 100% purity, TFA salt) as a white solid. LCMS (Method E): Rt=0.514 min, [M+H]⁺=1069.6. ¹H NMR (400 MHz, DMSO-d6) δ=12.59 (s, 1H), 8.85-8.63 (m, 1H), 8.29-8.23 (m, 1H), 8.15-8.04 (m, 1H), 7.99-7.94 (m, 1H), 7.93-7.87 (m, 1H), 7.85-7.81 (m, 1H), 7.53-7.40 (m, 3H), 7.39-7.31 (m, 1H), 7.24-7.22 (m, 2H), 4.43-4.27 (m, 4H), 4.24-4.13 (m, 1H), 4.00-3.91 (m, 1H), 3.91-3.81 (m, 1H), 3.67-3.61 (m, 1H), 3.60-3.47 (m, 4H), 3.36 (d, J=3.2 Hz, 2H), 3.30-3.24 (m, 1H), 3.21-3.12 (m, 3H), 3.11-3.03 (m, 2H), 3.02-2.92 (m, 1H), 2.90-2.79 (m, 1H), 2.58 (s, 1H), 2.43-2.36 (m, 2H), 2.15-2.01 (m, 9H), 1.92-1.76 (m, 8H), 1.68 (d, J=1.6 Hz, 5H), 1.63-1.52 (m, 5H), 1.51-1.41 (m, 2H), 1.21-1.03 (m, 5H), 1.03-0.95 (m, 1H). ¹⁹F NMR (377 MHz, DMSO-d6) δ=−73.40 (s, 1F), −119.74 (s, 1F), −120.23-−120.68 (m, 1F). SFC: Rt=3.424 min, 4.219 min

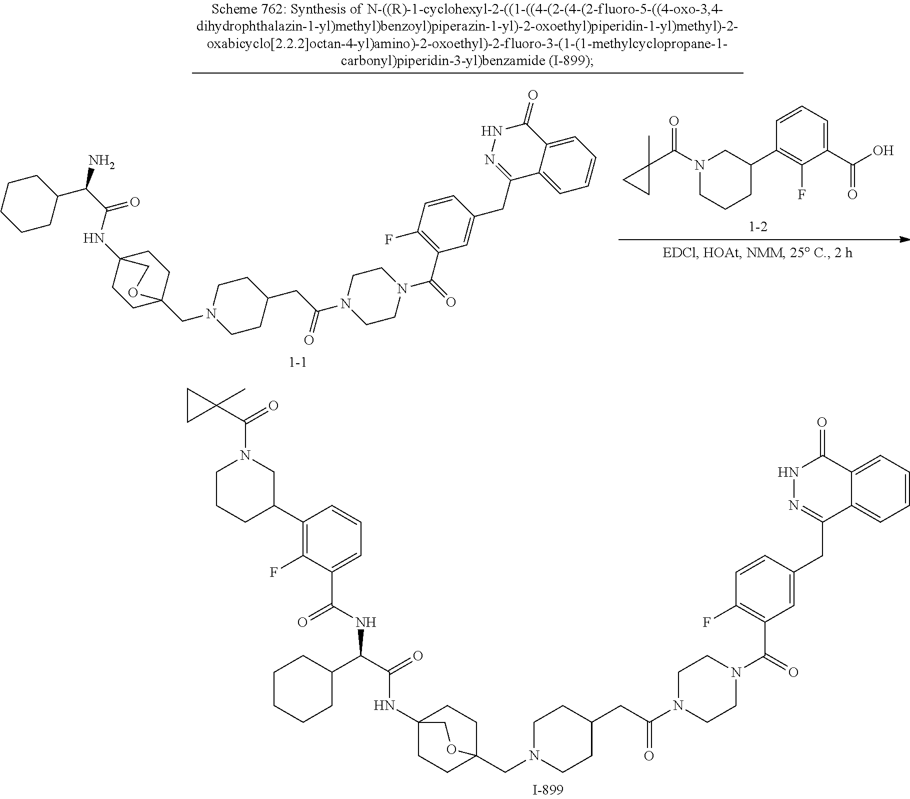

To a solution of Intermediate 1-2 (19.69 mg, 64.48 μmol, 1.3 eq) in DMF (1 mL) was added HOAt (13.50 mg, 99.21 μmol, 13.88 μL, 2 eq) and EDCI (19.02 mg, 99.21 μmol, 2 eq). The mixture was stirred at 25° C. for 30 min. Then Intermediate 1-1 (40 mg, 49.60 μmol, 1 eq, HCl salt) and NMM (25.09 mg, 248.01 μmol, 27.27 μL, 5 eq) was added and the mixture was stirred at 25° C. for 1 hr. The mixture was poured into 10 mL of water and extracted with EA (10 mL*2). The combined organic phase was dried over anhydrous Na₂SO₄, filtered and concentrated in vacuum. The residue was purified by reversed phase chromatography (26.8*125 mm, 80 g of XB—C18, 20-40 μm, 120 Å) Mobile phase: A for H₂O (0.1% NH₃·H₂O v/v) and B for acetonitrile; Gradient: B 5%-95% in 15 min; Flow rate: 40 mL/min; Column temperature: R.T. Wavelength: 220 nm/254 nm), and the eluent was concentrated and lyophilized to afford I-899 (12.54 mg, 11.86 μmol, 25.08% yield, 100% purity) as a white solid. LCMS (Method E): Rt=0.509 min, [M+H]⁺=1057.5. ¹H NMR (400 MHz, DMSO-d6) δ=12.59 (s, 1H), 8.26 (d, J=8.0 Hz, 1H), 8.16-8.04 (m, 1H), 7.99-7.94 (m, 1H), 7.89-7.87 (m, 1H), 7.86-7.80 (m, 1H), 7.66 (s, 1H), 7.54-7.42 (m, 3H), 7.36 (d, J=1.2 Hz, 1H), 7.28-7.20 (m, 2H), 4.37-4.29 (m, 5H), 3.86-3.74 (m, 2H), 3.65-3.59 (m, 1H), 3.59-3.49 (m, 3H), 3.36 (d, J=1.2 Hz, 2H), 3.20-3.10 (m, 2H), 2.99-2.79 (m, 5H), 2.24 (d, J=5.2 Hz, 1H), 2.20-2.11 (m, 3H), 1.91 (s, 1H), 1.86-1.76 (m, 7H), 1.74-1.66 (m, 4H), 1.58 (s, 9H), 1.23 (s, 3H), 1.20-1.03 (m, 7H), 1.02-0.95 (m, 1H), 0.85-0.77 (m, 2H), 0.57-0.50 (m, 2H). ¹⁹F NMR (377 MHz, DMSO-d6) δ=−119.75 (s, 1F), −120.44 (s, 1F). SFC: Rt=1.927 min, 2.323 min

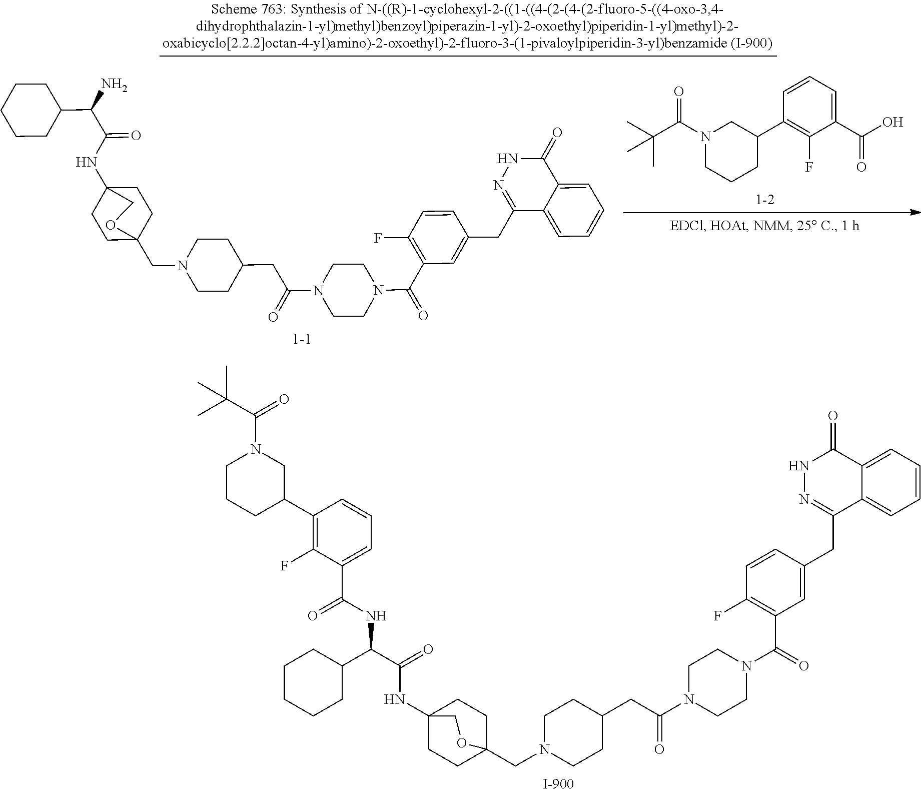 [SMILES: N[C@@H](C(=O)NC12CCC(CN3CCC(CC(=O)N4CCN(C(=O)c5cc(Cc6n[nH]c(=O)c7ccccc67)ccc5F)CC4)CC3)(CC1)OC2)C1CCCCC1 ; CC(C)(C)C(=O)N1CCCC(c2cccc(C(=O)O)c2F)C1 ; CC(C)(C)C(=O)N1CCCC(c2cccc(C(=O)N[C@@H](C(=O)NC34CCC(CN5CCC(CC(=O)N6CCN(C(=O)c7cc(Cc8n[nH]c(=O)c9ccccc89)ccc7F)CC6)CC5)(CC3)OC4)C3CCCCC3)c2F)C1]

To a solution of Intermediate 1-1 (30 mg, 37.20 μmol, 1 eq, HCl salt) in DMF (1 mL) was added Intermediate 1-2 (12.58 mg, 40.92 μmol, 1.1 eq), NMM (11.29 mg, 111.61 μmol, 12.27 μL, 3 eq), EDCI (14.26 mg, 74.40 μmol, 2 eq) and HOAt (10.13 mg, 74.40 μmol, 10.41 μL, 2 eq). The mixture was stirred at 25° C. for 1 hr. The reaction mixture was diluted with water (10 mL) and extracted with EA (10 mL*3). The combined organic layers were washed with sat·NaCl (10 mL), dried over Na₂SO₄, filtered and concentrated under reduced pressure to give a residue. The residue was purified by reversed phase chromatography (26.8*125 mm, 30 g of XB—C18, 20-40 μm, 120 Å) Mobile phase: A for H₂O (0.1% FA v/v) and B for acetonitrile; Gradient: B 5%-95% in 15 min; Flow rate: 40 mL/min; Column temperature: R.T. Wavelength: 220 nm/254 nm), and the eluent was concentrated and lyophilized to afford I-900 (16.02 mg, 14.49 μmol, 38.96% yield, 100% purity, FA salt) as a white solid. LCMS (Method E): Rt=0.532 min, [M+H]⁺=1059.7. ¹H NMR (400 MHz, DMSO-d6) δ=12.59 (s, 1H), 8.26 (d, J=7.6 Hz, 1H), 8.14 (s, 1H), 8.12-8.04 (m, 1H), 8.00-7.94 (m, 1H), 7.91-7.87 (m, 1H), 7.86-7.80 (m, 1H), 7.68 (s, 1H), 7.51-7.48 (m, 1H), 7.44 (d, J=4.8 Hz, 2H), 7.38-7.32 (m, 1H), 7.27-7.20 (m, 2H), 4.40-4.26 (m, 5H), 3.89-3.75 (m, 2H), 3.67-3.48 (m, 4H), 3.40-3.35 (m, 2H), 3.30-3.24 (m, 2H), 3.20-3.09 (m, 2H), 2.95-2.81 (m, 4H), 2.47 (d, J=2.0 Hz, 2H), 2.28-2.15 (m, 3H), 2.05-1.93 (m, 3H), 1.90-1.76 (m, 7H), 1.72-1.66 (m, 3H), 1.65-1.42 (m, 9H), 1.21 (s, 9H), 1.18-1.03 (m, 5H), 1.02-0.94 (m, 1H). ¹⁹F NMR (377 MHz, DMSO-d6) δ=−119.75 (br s, 1F), −120.16-−120.60 (m, 1F). SFC: Rt=3.101 min, 4.549 min.

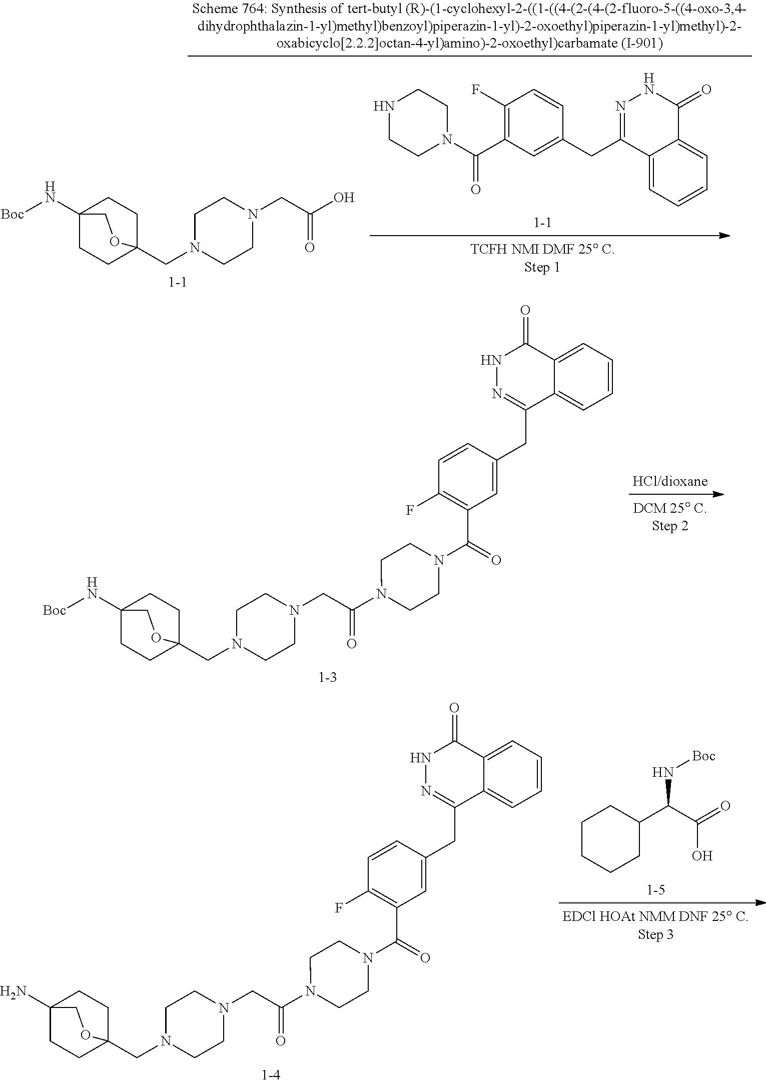 [SMILES: CC(C)(C)OC(=O)NC12CCC(CN3CCN(CC(=O)O)CC3)(CC1)OC2 ; O=C(c1cc(Cc2n[nH]c(=O)c3ccccc23)ccc1F)N1CCNCC1 ; CC(C)(C)OC(=O)NC12CCC(CN3CCN(CC(=O)N4CCN(C(=O)c5cc(Cc6n[nH]c(=O)c7ccccc67)ccc5F)CC4)CC3)(CC1)OC2 ; NC12CCC(CN3CCN(CC(=O)N4CCN(C(=O)c5cc(Cc6n[nH]c(=O)c7ccccc67)ccc5F)CC4)CC3)(CC1)OC2 ; CC(C)(C)OC(=O)N[C@@H](C(=O)O)C1CCCCC1]

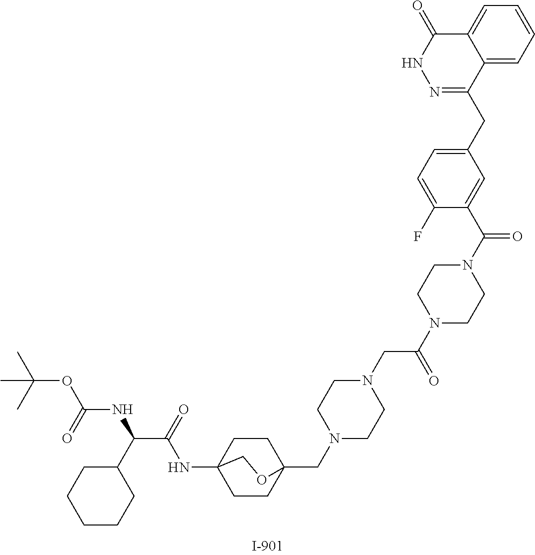 [SMILES: CC(C)(C)OC(=O)N[C@@H](C(=O)NC12CCC(CN3CCN(CC(=O)N4CCN(C(=O)c5cc(Cc6n[nH]c(=O)c7ccccc67)ccc5F)CC4)CC3)(CC1)OC2)C1CCCCC1]

### Step 1: Synthesis of Intermediate 1-3

To a solution of 2-[4-[[4-(tert-butoxycarbonylamino)-2-oxabicyclo[2.2.2]octan-1-yl]methyl]piperazin-1-yl]acetic acid (100 mg, 260.77 μmol, 1 eq) and 4-[[4-fluoro-3-(piperazine-1-carbonyl)phenyl]methyl]-2H-phthalazin-1-one (95.54 mg, 260.77 μmol, 1 eq) and in DMF (1 mL) was added TCFH (146.33 mg, 521.54 μmol, 2 eq) and NMI (107.05 mg, 1.30 mmol, 103.93 μL, 5 eq). The mixture was stirred at 25° C. for 1 hr. The reaction was quenched with H₂O (10 mL), and extracted with ethyl acetate 15 mL (5 mL*3). The organic phase was washed with a saturated aqueous NaCl solution (10 mL), then dried over Na₂SO₄, filtered and concentrated under reduced pressure to give a residue. The residue was purified by reversed phase chromatogrpahy (26.8*125 mm, of XB—C18, 20-40 m, 80 Å) Mobile phase: A for H₂O (0.1% FA v/v) and B for acetonitrile; Gradient: B 0%-100% in 20 min; Flow rate: 50 mL/min; Column temperature: R.T. Wavelength: 220 nm/254 nm), then concentrated to remove organic solvents, and lyophilized to give product. Compound tert-butyl N-[1-[[4-[2-[4-[2-fluoro-5-[(4-oxo-3H-phthalazin-1-yl)methyl]benzoyl]piperazin-1-yl]-2-oxoethyl]piperazin-1-yl]methyl]-2-oxabicyclo[2.2.2]octan-4-yl]carbamate (50 mg, 66.95 μmol, 25.68% yield, 98% purity) was obtained as a white solid. LCMS (Method E): Rt=0.478 min, [M+H]⁺=732.4.

### Step 2: Synthesis of Intermediate 1-4

To a solution of Intermediate 1-3 (50 mg, 68.32 μmol, 1 eq) in DCM (0.3 mL) was added HCl/dioxane (2 M, 0.5 mL, 14.64 eq). The mixture was stirred at 25° C. for 1 hr. The reaction mixture was concentrated under reduced pressure to give the crude product. The crude product 4-[[3-[4-[2-[4-[(4-amino-2-oxabicyclo[2.2.2]octan-1-yl)methyl]piperazin-1-yl]acetyl]piperazine-1-carbonyl]-4-fluoro-phenyl]methyl]-2H-phthalazin-1-one (50 mg, crude, HCl salt) was used in the next step without further purification. 4-[[3-[4-[2-[4-[(4-amino-2-oxabicyclo[2.2.2]octan-1-yl)methyl]piperazin-1-yl]acetyl]piperazine-1-carbonyl]-4-fluoro-phenyl]methyl]-2H-phthalazin-1-one (50 mg, crude, HCl salt) was obtained as a white solid. LCMS (Method D): Rt=0.358 min, [M+H]⁺=632.4.

### Step 3: Synthesis of I-901

To a solution of Intermediate 1-4 and (2R)-2-(tertbutoxycarbonylamino)-2-cyclohexyl-acetic acid (28.88 mg, 112.24 μmol, 1.5 eq) in DMF (0.5 mL) was added EDCI (71.72 mg, 374.14 μmol, 5 eq), HOAt (30.55 mg, 224.48 μmol, 31.40 μL, 3 eq) and NMM (75.69 mg, 748.28 μmol, 82.27 μL, 10 eq). The mixture was stirred at 25° C. for 1 hr. The reaction was quenched with H₂O (5 mL) and extracted with ethyl acetate (5 mL*3). The organic phase was washed with a saturated aqueous NaCl solution (15 mL), dried over Na₂SO₄, filtered and concentrated under reduced pressure to give a residue. The crude product was purified by prep-HPLC (column: Phenomenex luna C18 150*25 mm*10 um; mobile phase: [water (FA)-ACN]; gradient: 18%-48% B over 15 min) and the eluent was concentrated to remove MeCN and then lyophilization to give product. Compound tert-butyl N-[(1R)-1-cyclohexyl-2-[[1-[[4-[2-[4-[2-fluoro-5-[(4-oxo-3H-phthalazin-1-yl)methyl]benzoyl]piperazin-1-yl]-2-oxo-ethyl]piperazin-1-yl]methyl]-2-oxabicyclo[2.2.2]octan-4-yl]amino]-2-oxo-ethyl]carbamate (20 mg, 19.96 μmol, 34.77% yield, 91.53% purity, FA salt) was obtained as a white solid. LCMS (Method E): Rt=0.476 min, [M+H]⁺=871.5. SFC: Rt=1.620 min, ee %=100%. ¹H NMR (400 MHz, METHANOL-d4) δ=8.51 (s, 1H), 8.37 (d, J=7.6 Hz, 1H), 7.99-7.92 (m, 1H), 7.91-7.79 (m, 2H), 7.49 (d, J=5.2 Hz, 1H), 7.37 (m, 1H), 7.16 (m, 1H), 4.39 (s, 2H), 4.02-3.89 (m, 2H), 3.82-3.63 (m, 5H), 3.58-3.47 (m, 2H), 3.25 (s, 2H), 2.79-2.48 (m, 8H), 2.33 (d, J=12.4 Hz, 2H), 2.19-2.05 (m, 2H), 2.03-1.86 (m, 4H), 1.85-1.49 (m, 9H), 1.43 (s, 9H), 1.36-0.87 (m, 6H). ¹⁹F NMR (377 MHz, METHANOL-d4) δ=−76.96 (s, 1F), −120.75 (d, J=22.4 Hz, 1F).

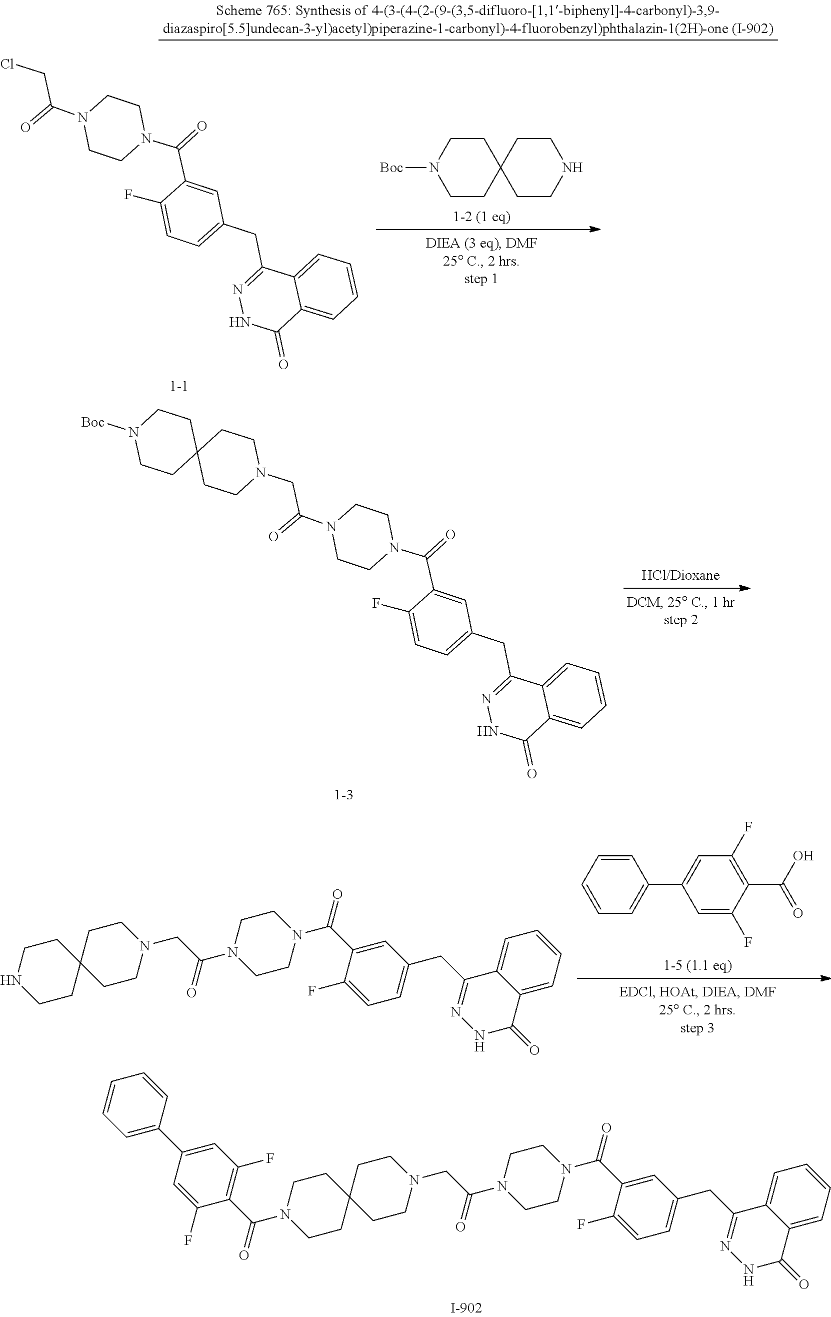 [SMILES: O=C(CCl)N1CCN(C(=O)c2cc(Cc3n[nH]c(=O)c4ccccc34)ccc2F)CC1 ; CC(C)(C)OC(=O)N1CCC2(CCNCC2)CC1 ; CC(C)(C)OC(=O)N1CCC2(CCN(CC(=O)N3CCN(C(=O)c4cc(Cc5n[nH]c(=O)c6ccccc56)ccc4F)CC3)CC2)CC1 ; O=C(CN1CCC2(CCNCC2)CC1)N1CCN(C(=O)c2cc(Cc3n[nH]c(=O)c4ccccc34)ccc2F)CC1 ; O=C(O)c1c(F)cc(-c2ccccc2)cc1F ; O=C(CN1CCC2(CC1)CCN(C(=O)c1c(F)cc(-c3ccccc3)cc1F)CC2)N1CCN(C(=O)c2cc(Cc3n[nH]c(=O)c4ccccc34)ccc2F)CC1]

### Step 1: Synthesis of Intermediate 1-3

To a solution of intermediate 1-2 (287.18 mg, 1.13 mmol, 1 eq) in DMF (5 mL) was added intermediate 1-1 (500 mg, 1.13 mmol, 1 eq) and DIEA (218.87 mg, 1.69 mmol, 294.97 μL, 1.5 eq) and the reaction mixture was stirred at 25° C. for 2 hrs. The reaction mixture was quenched by water (10 mL), and extracted by DCM (10 mL*3). The organic phase was washed by brine (10 mL*2). The organic phase was combined and concentrated to afford the crude. The crude product was purified by reverse phase chromatography (80 g of XB—C18, 20-40 μm, 120 Å) Mobile phase: A for H₂O (0.1% FAv/v) and B for acetonitrile; Gradient: B5%-100% in 30 min; Flow rate: 100 ml/min; Column temperature: R.T. Wavelength: 220 nm/254 nm) and the eluent was then concentrated to remove organic solvents and adjusted to pH=7 by a saturated solution of NaHCO₃ (30 mL), and extracted by DCM (50 mL*3). The organic phase was combined, dried by anhydrous Na₂SO₄ and concentrated to afford the product. Intermediate 1-3 (500 mg, 726.42 μmol, 64.34% yield, 96% purity) was obtained as a yellow solid. LCMS (Method D): Rt=0.425 min, [M+H]⁺=661.4. ¹H NMR (400 MHz, CHLOROFORM-d) δ=10.64-10.22 (m, 1H), 8.60-8.34 (m, 1H), 7.86-7.63 (m, 3H), 7.42-7.29 (m, 2H), 7.05 (t, J=8.4 Hz, 1H), 4.29 (s, 2H), 3.88-3.66 (m, 4H), 3.65-3.49 (m, 2H), 3.36 (d, J=4.0 Hz, 4H), 3.27 (br s, 1H), 3.23-3.14 (m, 2H), 2.46 (br d, J=4.8 Hz, 3H), 1.71 (br s, 3H), 1.57-1.36 (m, 16H).

### Step 2: Synthesis of Intermediate 1-4

To a solution of Intermediate 1-3 (400 mg, 605.35 μmol, 1 eq) in DCM (4 mL) was added HCl/dioxane (4 M, 4 mL, 26.43 eq). The reaction suspension was stirred at 25° C. for 1 hr. The reaction suspension was concentrated to give the residue. The mixture was purified by reversed phase HPLC (0.1% FA, 15%), and the major peak was collected and lyophilized to give the desired product, which was diluted with a sat.aq·Na₂CO₃ solution (10 mL) and extracted with DCM (20*3 mL). The combined organic layers were dried over Na₂SO₄, filtered and concentrated under reduced pressure to give a residue. Intermediate 1-4 (250 mg, 445.90 μmol, 73.66% yield) was obtained as a white solid. LCMS (Method E): Rt=0.354 min, [M+H]⁺=561.2.

### Step 3: Synthesis of I-902

To a mixture of Intermediate 1-5 (105.68 mg, 451.25 μmol, 1.1 eq) in DMF (5 mL) was added EDCI (235.92 mg, 1.23 mmol, 3 eq), HOAt (83.76 mg, 615.35 μmol, 86.08 μL, 1.5 eq) and NMM (207.47 mg, 2.05 mmol, 225.51 μL, 5 eq). Then Intermediate 1-4 (230 mg, 410.23 μmol, 1 eq) was added. The reaction solution was stirred at 25° C. for 2 hrs. The reaction solution was diluted with water (5 mL) and extracted with EA (5 mL*3). The combined organic layers were washed with brine (5 mL), dried over Na₂SO₄, filtered and concentrated under reduced pressure to give a residue. The residue was purified by prep-HPLC (0.1% FA condition), the major peak was collected and concentrated to remove MeCN, and then lyophilized to get the desired product. I-902 (195.92 mg, 237.82 μmol, 57.97% yield, 99.884% purity, FA salt) was obtained as a white solid. LCMS (Method E): Rt=0.473 min, [M+H]⁺=777.4. ¹H NMR (400 MHz, DMSO-d₆) δ=12.60 (s, 1H), 8.27-8.25 (m, 1H), 8.16 (s, 1H), 8.00-7.94 (m, 1H), 7.89 (t, J=7.6 Hz, 1H), 7.82 (d, J=4.0 Hz, 1H), 7.80-7.75 (m, 2H), 7.58 (d, J=9.2 Hz, 2H), 7.53-7.41 (m, 4H), 7.40-7.33 (m, 1H), 7.23 (t, J=8.8 Hz, 1H), 4.33 (s, 2H), 3.69-3.55 (m, 5H), 3.52-3.44 (m, 2H), 3.35 (s, 1H), 3.26 (br s, 2H), 3.19-3.06 (m, 4H), 2.44-2.32 (m, 4H), 1.55-1.31 (m, 8H). ¹⁹F NMR (377 MHz, DMSO-d₆) δ=−113.83 (s, 2F), −119.77 (br d, J=13.5 Hz, 1F).

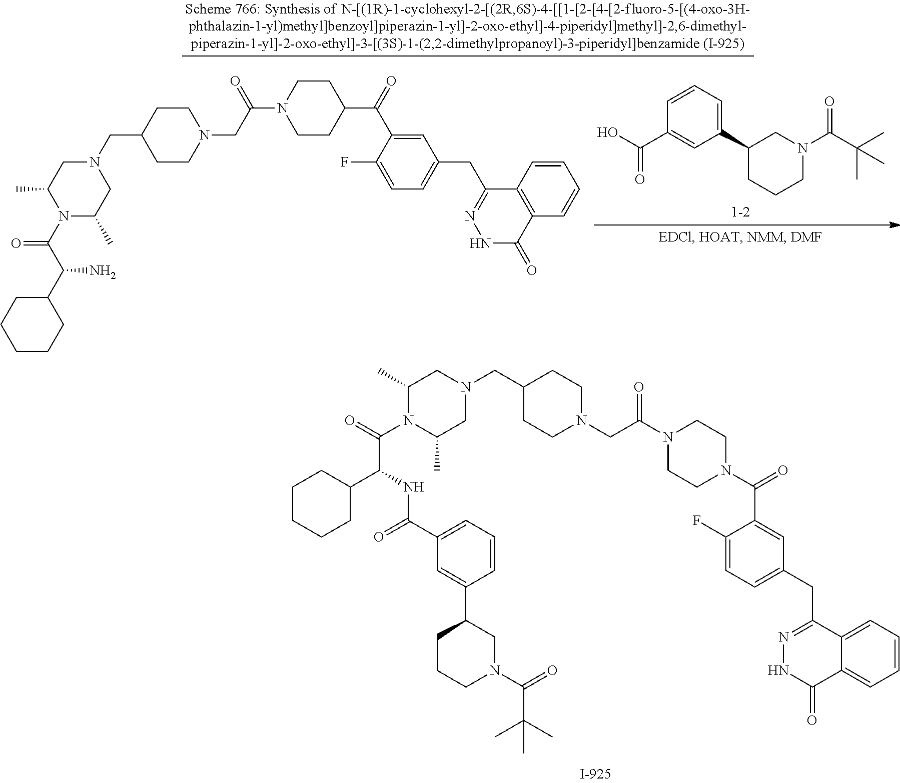 [SMILES: C[C@@H]1CN(CC2CCN(CC(=O)N3CCC(C(=O)c4cc(Cc5n[nH]c(=O)c6ccccc56)ccc4F)CC3)CC2)C[C@H](C)N1C(=O)[C@H](N)C1CCCCC1 ; CC(C)(C)C(=O)N1CCC[C@@H](c2cccc(C(=O)O)c2)C1 ; C[C@@H]1CN(CC2CCN(CC(=O)N3CCN(C(=O)c4cc(Cc5n[nH]c(=O)c6ccccc56)ccc4F)CC3)CC2)C[C@H](C)N1C(=O)[C@H](NC(=O)c1cccc([C@@H]2CCCN(C(=O)C(C)(C)C)C2)c1)C1CCCCC1]

To a solution of intermediate 1-1 (60.00 mg, 79.27 μmol, 1 eq) and intermediate 1-2 (25.23 mg, 87.19 μmol, 1.1 eq) in DMF (1 mL) was added EDCI (45.59 mg, 237.80 μmol, 3 eq) NMM (64.14 mg, 634.12 μmol, 69.72 μL, 8 eq) and HOAt (10.79 mg, 79.27 μmol, 11.09 μL, 1 eq). The mixture was stirred at 25° C. for 1 hr. The mixture was concentrated under reduced pressure to give a residue. The residue was purified by prep-HPLC (column: Phenomenex luna C18 150*25 mm*10 um; mobile phase: [water (FA)-ACN]; gradient: 23%-43% B over 10 min). I-925 (35 mg, 33.90 μmol, 42.77% yield) was obtained as a white solid LCMS (Method E): Rt=0.490 min, [M+H]⁺=1028.6. SFC: Rt=1.823, 2.322 min, ee %=29.940% ¹H NMR (400 MHz, DMSO-d6) δ=12.59 (s, 1H), 8.66-8.23 (m, 2H), 8.02-7.70 (m, 5H), 7.39 (br s, 4H), 7.26-7.20 (m, 1H), 4.85-4.61 (m, 1H), 4.55-4.38 (m, 1H), 4.33 (s, 3H), 4.30-4.18 (m, 1H), 3.69-3.55 (m, 4H), 3.50-3.35 (m, 8H), 3.15 (br d, J=8.4 Hz, 4H), 3.08 (br s, 1H), 2.90-2.60 (m, 7H), 2.12-2.03 (m, 2H), 2.01-1.89 (m, 4H), 1.82-1.66 (m, 7H), 1.61 (br d, J=2.0 Hz, 2H), 1.47 (br d, J=12.8 Hz, 1H), 1.41-1.33 (m, 3H), 1.20 (s, 9H), 1.16 (br d, J=6.4 Hz, 3H), 1.13-0.88 (m, 4H). ¹⁹F NMR (377 MHz, DMSO-d6) δ=−73.42 (s, 1F), −117.82-−121.69 (m, 1F).

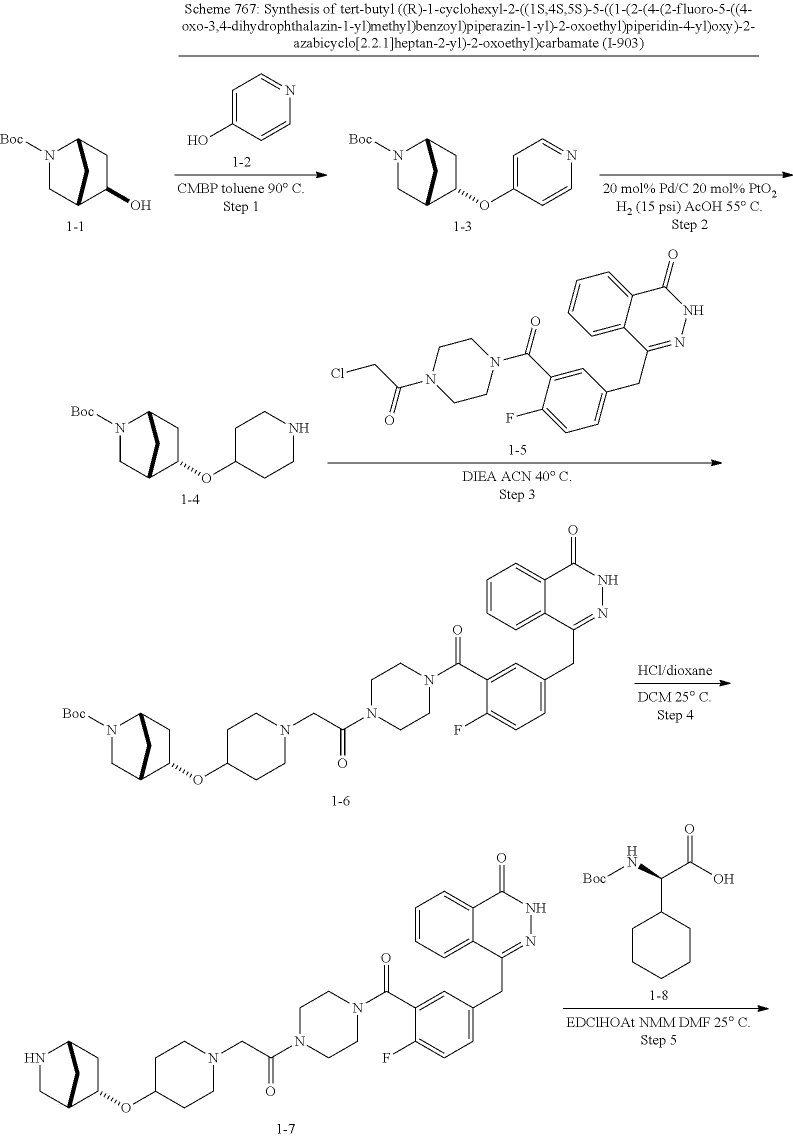

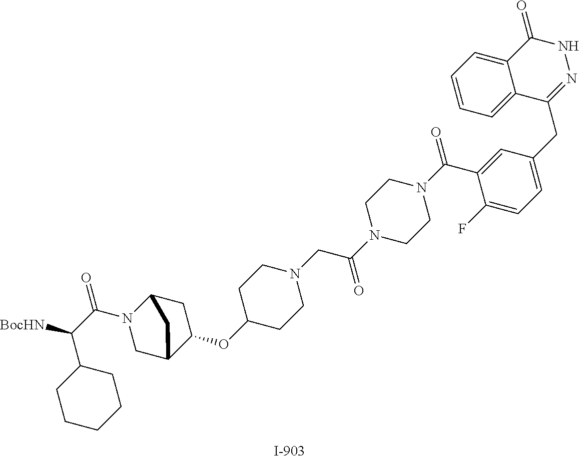

### Step 1: Synthesis of Intermediate 1-3

To a solution of tert-butyl 5-hydroxy-2-azabicyclo [2.2.1]heptane-2-carboxylate (500 mg, 2.34 mmol, 1 eq), pyridin-4-ol (222.95 mg, 2.34 mmol, 1 eq) in toluene (5 mL) was added 2-(tributyl-phosphanylidene) acetonitrile (1.70 g, 7.03 mmol, 3 eq). The mixture was stirred at 90° C. for 12 hr. The mixture was diluted with H₂O (5 mL) and extracted with EA (5 mL*2). The combined organic layers were dried over Na₂SO₄, filtered and concentrated under reduced pressure to give a residue. The filtrate was purified by reversed-phase HPLC (0.1% FA condition). The eluent was concentrated under reduced pressure to remove ACN and then lyophilized to give the product. Compound tert-butyl 5-(4-pyridyloxy)-2-azabicyclo[2.2.1]heptane-2-carboxylate (170 mg, 577.88 μmol, 24.65% yield, 98.7% purity) was obtained as a yellow gum. LCMS (Method D): Rt=0.331 min, [M+Na]⁺=291.1. ¹H NMR (400 MHz, METHANOL-d4) δ=8.44-8.29 (m, 2H), 8.24-8.17 (m, 1H), 7.07-7.00 (m, 2H), 5.03-4.95 (m, 1H), 4.21 (s, 1H), 3.69-3.59 (m, 1H), 3.25-3.16 (m, 1H), 2.99 (s, 1H), 2.36-2.23 (m, 1H), 1.88-1.79 (m, 1H), 1.77-1.70 (m, 1H), 1.55-1.48 (m, 1H), 1.47-1.43 (m, 9H).

### Step 2: Synthesis of Intermediate 1-4

To a solution of Intermediate 1-3 (150 mg, 516.61 μmol, 1 eq) in AcOH (2 mL) was added Pd/C (75 mg, 10% purity) and PtO₂ (75 mg). The flask was degassed and purged with H₂ 3 times, then the mixture was stirred at 55° C. for 12 hr under H₂ (15 psi) atmosphere. The mixture was filtered and the filter cake was washed with MeOH (1 mL*3), then the filtrate was concentrated under reduced pressure to give a residue. The residue was added to NH₃·H₂O (2 mL) to adjust the pH to 8. The mixture was diluted with H₂O (2 mL) and extracted with DCM 2 mL (1 mL*2). The combined organic layers were dried over Na₂SO₄, filtered and concentrated under reduced pressure to give a residue. The crude product tert-butyl 5-(4-piperidyloxy)-2-azabicyclo [2.2.1]heptane-2-carboxylate (110 mg, 369.26 μmol, 71.48% yield, 99.5% purity) as colorless oil was used in the next step without further purification. LCMS (Method D): Rt=0.308 min, [M+H]⁺=297.2.

### Step 3: Synthesis of Intermediate 1-6

To a solution of Intermediate 1-4 (110 mg, 371.12 μmol, 1 eq) and 4-[[3-[4-(2-chloroacetyl) piperazine-1-carbonyl]-4-fluoro-phenyl]methyl]-2H-phthalazin-1-one (164.36 mg, 371.12 μmol, 1 eq) in ACN (1.5 mL) was added DIEA (143.89 mg, 1.11 mmol, 193.92 μL, 3 eq). The mixture was stirred at 40° C. for 12 hr. The mixture was diluted with H₂O (2 mL) and extracted with EA (2 mL*2). The combined organic layers were dried over Na₂SO₄, filtered and concentrated under reduced pressure to give a residue. The residue was purified by flash silica gel chromatography (ISCO; 20 g SepaFlash Silica Flash Column, Eluent of 0˜10% MeOH/Ethyl acetate gradient @60 mL/min). The eluent was concentrated under reduced pressure to give the product. Compound tert-butyl 5-[[1-[2-[4-[2-fluoro-5-[(4-oxo-3H-phthalazin-1-yl) methyl]benzoyl]piperazin-1-yl]-2-oxoethyl]-4-piperidyl]oxy]-2-azabicyclo [2.2.1]heptane-2-carboxylate (130 mg, 184.97 gmol, 49.84% yield, 100% purity) was obtained as a yellow solid. LCMS (Method D): Rt=0.355 min, [M+H]⁺=703.3. ¹H NMR (400 MHz, METHANOL-d4) δ=8.42-8.37 (m, 1H), 8.00-7.95 (m, 1H), 7.93-7.82 (m, 2H), 7.51 (d, J=2.0 Hz, 1H), 7.43-7.37 (m, 1H), 7.23-7.15 (m, 1H), 4.63 (s, 1H), 4.41 (s, 2H), 4.22-4.13 (m, 1H), 4.09 (s, 1H), 3.84-3.67 (m, 4H), 3.61-3.50 (m, 3H), 3.44-3.38 (m, 1H), 3.29-3.19 (m, 2H), 3.17-3.08 (m, 1H), 2.84-2.66 (m, 3H), 2.34-2.20 (m, 2H), 2.03-1.86 (m, 3H), 1.73-1.54 (m, 4H), 1.49-1.45 (m, 9H), 1.40-1.30 (m, 2H). ¹⁹F NMR (376 MHz, METHANOL-d4) δ=−120.764.

### Step 4: Synthesis of Intermediate 1-7

The solution of Intermediate 1-6 (240 mg, 341.48 μmol, 1 eq) in DCM (1 mL) and HCl/dioxane (2 M) was stirred at 25° C. for 1 h. The mixture was concentrated in vacuum. The crude product 4-[[3-[4-[2-[4-(2-azabicyclo[2.2.1]heptan-5-yloxy)-1-piperidyl]acetyl]piperazine-1-carbonyl]-4-fluoro-phenyl]methyl]-2H-phthalazin-1-one (220 mg, crude, HCl salt) as a white solid was used in the next step without further purification. LCMS (Method D): Rt=0.243 min, [M+H]⁺=603.3.

### Step 5: Synthesis of I-903

To a solution of (2R)-2-(tert-butoxycarbonylamino)-2-cyclohexyl-acetic acid (88.57 mg, 344.20 μmol, 1 eq) in DMF (2.5 mL) was added EDCI (197.95 mg, 1.03 mmol, 3 eq), HOAt (46.85 mg, 344.20 μmol, 48.15 μL, 1 eq), Intermediate 1-7 (220 mg, 344.20 μmol, 1 eq, HCl salt) and NMM (174.08 mg, 1.72 mmol, 189.21 μL, 5 eq). The mixture was stirred at 25° C. for 1 hr. The mixture was diluted with H₂O (10 mL) and extracted with EA (10 mL*2). The combined organic layers were washed with brine (5 mL), dried over Na₂SO₄, filtered and concentrated under reduced pressure to give a residue. The residue was purified by flash silica gel chromatography (ISCO; 12 g SepaFlash Silica Flash Column, Eluent of 0˜15% MeOH/Ethyl acetate gradient @60 mL/min). The crude product tert-butyl N-[(1R)-1-cyclohexyl-2-[5-[[1-[2-[4-[2-fluoro-5-[(4-oxo-3H-phthalazin-1-yl)methyl]benzoyl]piperazin-1-yl]-2-oxo-ethyl]-4-piperidyl]oxy]-2-azabicyclo [2.2.1]heptan-2-yl]-2-oxo-ethyl]carbamate (230 mg, 271.30 μmol, 95.18% yield, 99.32% purity) was lyophilized to afford desired product. Compound tert-butyl N-[(1R)-1-cyclohexyl-2-[5-[[1-[2-[4-[2-fluoro-5-[(4-oxo-3H-phthalazin-1-yl)methyl]benzoyl]piperazin-1-yl]-2-oxo-ethyl]-4-piperidyl]oxy]-2-azabicyclo[2.2.1]heptan-2-yl]-2-oxo-ethyl]carbamate (230 mg, 271.30 μmol, 95.18% yield, 99.32% purity) was obtained as a white solid. LCMS (Method D): Rt=0.397 min, [M+H]⁺=842.4. SFC: Rt=4.467 min. ¹H NMR (400 MHz, METHANOL-d4) δ=8.37 (d, J=7.6 Hz, 1H), 8.00-7.93 (m, 1H), 7.91-7.80 (m, 2H), 7.49 (s, 1H), 7.37 (m, 1H), 7.17 (m, 1H), 6.69-6.43 (m, 1H), 4.62-4.47 (m, 1H), 4.39 (s, 2H), 4.26-4.16 (m, 1H), 4.14-3.97 (m, 1H), 3.93-3.64 (m, 5H), 3.63-3.42 (m, 5H), 3.26-3.10 (m, 1H), 2.99-2.73 (m, 3H), 2.67-2.32 (m, 2H), 2.11-1.88 (m, 3H), 1.83-1.54 (m, 10H), 1.52-1.37 (m, 10H), 1.36-1.16 (m, 4H), 1.12-0.93 (m, 2H). ¹⁹F NMR (377 MHz, METHANOL-d4) δ=−120.61-−120.83 (m, 1F).

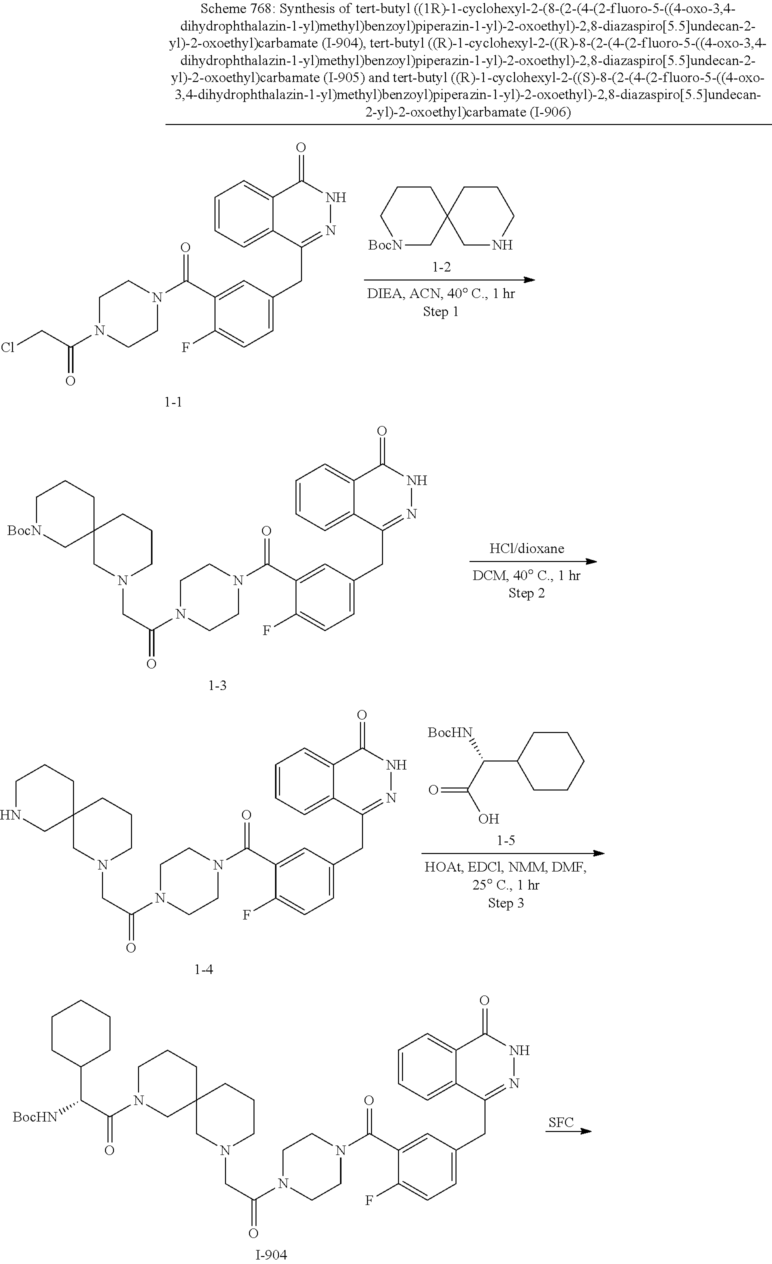

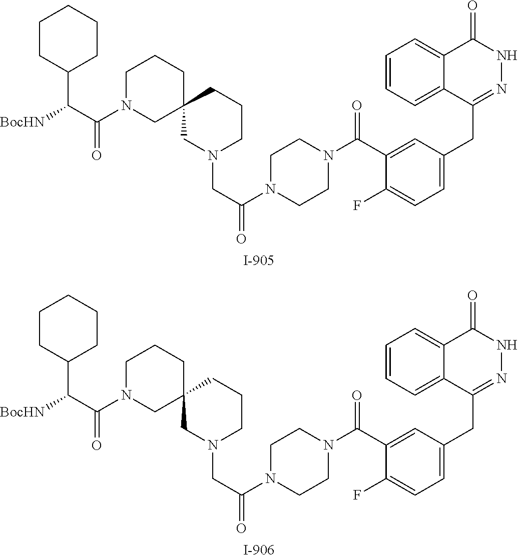 [SMILES: CC(C)(C)OC(=O)N[C@@H](C(=O)N1CCC[C@@]2(CCCN(CC(=O)N3CCN(C(=O)c4cc(Cc5n[nH]c(=O)c6ccccc56)ccc4F)CC3)C2)C1)C1CCCCC1 ; CC(C)(C)OC(=O)N[C@@H](C(=O)N1CCC[C@]2(CCCN(CC(=O)N3CCN(C(=O)c4cc(Cc5n[nH]c(=O)c6ccccc56)ccc4F)CC3)C2)C1)C1CCCCC1]

### Step 1: Synthesis of Intermediate 1-3

To a solution of Intermediate 1-1 (922.76 mg, 2.08 mmol, 1 eq) in ACN (10 mL) was added Intermediate 1-2 (530 mg, 2.08 mmol, 1 eq) and DIEA (807.87 mg, 6.25 mmol, 1.09 mL, 3 eq). The mixture was stirred at 40° C. for 3 hr. The mixture was diluted with H₂O (10 mL) and extracted with EA (10 mL*2). The combined organic layers were dried over Na₂SO₄, filtered and concentrated under reduced pressure to give a residue. The residue was purified by flash silica gel chromatography (ISCO; 40 g SepaFlash Silica Flash Column, Eluent of 0-100% Ethyl acetate/Petroleum ether@60 mL/min). The eluent was concentrated under reduced pressure. Intermediate 1-3 (1.2 g, 1.74 mmol, 83.56% yield, 95.867% purity) as yellow solid was obtained. LCMS (Method D): Rt=0.353 min, [M+H]⁺=661.3

### Step 2: Synthesis of Intermediate 1-4

A solution of Intermediate 1-3 (1.2 g, 1.82 mmol, 1 eq) in CH₂Cl₂ (6 mL) and HCl/dioxane (2 M, 12 mL) was stirred at 25° C. for 1 hr. The mixture was concentrated in vacuum. Intermediate 1-4 (1.1 g, crude, HCl) as a white solid was obtained. LCMS (Method D): Rt=0.230 min, [M+H]⁺=561.3.

### Step 3: Synthesis of I-904

To a solution of Intermediate 1-5 (474.04 mg, 1.84 mmol, 1 eq) in DMF (12 mL) was added EDCI (1.06 g, 5.53 mmol, 3 eq), HOAt (250.74 mg, 1.84 mmol, 257.70 μL, 1 eq), Intermediate 1-4 (1.1 g, 1.84 mmol, 1 eq, HCl salt) and NMM (931.68 mg, 9.21 mmol, 1.01 mL, 5 eq). The mixture was stirred at 25° C. for 1 hr. The mixture was diluted with H₂O (10 mL) and extracted with EA (15 mL*2). The combined organic layers were washed with brine 20 mL (10 mL*2), dried over Na₂SO₄, filtered and concentrated under reduced pressure to give a residue. The residue was purified by flash silica gel chromatography (ISCO; 12 g SepaFlash Silica Flash Column, Eluent of 0˜8% MeOH/Ethylacetate gradient @60 mL/min). The eluent was concentrated under reduced pressure to give the product. I-904 (1.4 g, 1.70 mmol, 92.02% yield, 96.86% purity) as yellow gum was obtained. LCMS (Method E): Rt=0.522 min, [M+H]⁺=800.6. SFC: Rt=4.029 & 5.377 min. ¹H NMR (400 MHz, CHLOROFORM-d) δ=12.00-10.40 (m, 1H), 8.61-8.41 (m, 1H), 7.89-7.81 (m, 1H), 7.81-7.68 (m, 2H), 7.40-7.27 (m, 2H), 7.14-6.96 (m, 1H), 5.35 (br dd, J=5.8, 8.6 Hz, 1H), 4.60-4.42 (m, 1H), 4.40-4.19 (m, 2H), 4.17-3.60 (m, 5H), 3.61-3.23 (m, 6H), 3.18 (s, 2H), 2.82-2.05 (m, 3H), 2.04-1.84 (m, 1H), 1.78-1.76 (m, 1H), 1.80 (s, 3H), 1.75-1.51 (m, 8H), 1.51-1.33 (m, 9H), 1.32-1.24 (m, 1H), 1.22 (br s, 2H), 1.19-0.87 (m, 5H). ¹⁹F NMR (377 MHz, CHLOROFORM-d) δ=−117.43-−119.04 (m, 1F).

### Step 4: Synthesis of I-905 & I-906

I-904 was separated by prep-HPLC (column: ChiralPak IH, 250*50 mm, 10 um; mobile phase: [Hexane-ETOH: ACN (4:1) (0.1IPAM)]; B %: 35%, isocratic elution mode) and the eluent was concentrated in vacuum. I-905 (400 mg, 497.18 μmol, 39.77% yield, 99.433% purity) as yellow solid was obtained. I-906 (450 mg, 544.70 μmol, 43.57% yield, 96.832% purity) as yellow solid was obtained. I-905: LCMS (Method E): Rt=0.517 min, [M+H]⁺=800.5. SFC: Rt=3.99 min. Peak 1 was arbitrarily assigned to I-905 and peak 2 was arbitrarily assigned to I-905. ¹H NMR (400 MHz, CHLOROFORM-d) δ=11.60-10.41 (m, 1H), 8.53-8.43 (m, 1H), 7.83 (br s, 1H), 7.80-7.68 (m, 2H), 7.38-7.28 (m, 2H), 7.07 (td, J=8.8, 12.8 Hz, 1H), 6.40-5.17 (m, 1H), 4.59-4.41 (m, 1H), 4.39-4.20 (m, 2H), 3.45 (br s, 11H), 3.35-3.15 (m, 2H), 3.11-2.21 (m, 4H), 2.11-1.90 (m, 3H), 1.77-1.54 (m, 9H), 1.43-1.32 (m, 8H), 1.24-0.90 (m, 6H). ¹⁹F NMR (377 MHz, CHLOROFORM-d) δ=−116.31-−119.65 (m, 1F). I-906: LCMS (Method E): Rt=0.524 min, [M+H]⁺=800.5. SFC: Rt=5.386 min. ¹H NMR (400 MHz, CHLOROFORM-d) δ=11.94-10.21 (m, 1H), 8.56-8.42 (m, 1H), 7.90-7.82 (m, 1H), 7.81-7.68 (m, 2H), 7.40-7.28 (m, 2H), 7.13-7.00 (m, 1H), 6.82-4.96 (m, 1H), 4.58-4.43 (m, 1H), 4.41-4.20 (m, 2H), 4.16-3.19 (m, 11H), 3.17-2.88 (m, 2H), 2.82-2.09 (m, 3H), 1.94-1.70 (m, 5H), 1.69-1.52 (m, 8H), 1.51-1.29 (m, 8H), 1.25-1.12 (m, 8H). ¹⁹F NMR (377 MHz, CHLOROFORM-d) δ=−115.47-−120.21 (m, 1F).

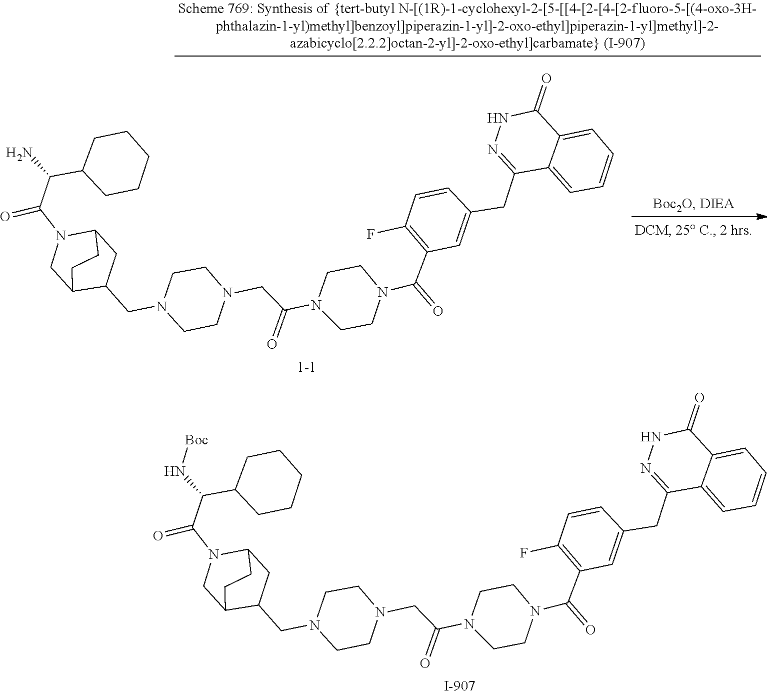 [SMILES: N[C@@H](C(=O)N1CC2CCC1CC2CN1CCN(CC(=O)N2CCN(C(=O)c3cc(Cc4n[nH]c(=O)c5ccccc45)ccc3F)CC2)CC1)C1CCCCC1 ; CC(C)(C)OC(=O)N[C@@H](C(=O)N1CC2CCC1CC2CN1CCN(CC(=O)N2CCN(C(=O)c3cc(Cc4n[nH]c(=O)c5ccccc45)ccc3F)CC2)CC1)C1CCCCC1]

To a solution of Intermediate 1-1 (100 mg, 132.46 μmol, 1 eq) in DCM (1 mL) was added DIEA (51.36 mg, 397.39 μmol, 69.22 μL, 3 eq), followed by Boc₂O (43.36 mg, 198.69 μmol, 45.65 μL, 1.5 eq). The reaction mixture was stirred at 25° C. for 2 hrs. The reaction solution was diluted with H₂O (2 mL) and extracted with DCM (2 mL*3). The combined organic layers were dried over anhydrous Na₂SO₄, filtered and concentrated under reduced pressure to give a residue. The residue was purified by reversed-phase HPLC (0.1% FA condition), the major peak was collected and concentrated to remove the solvent, then lyophilized to give I-907 (55.17 mg, 60.78 μmol, 45.89% yield, 99.275% purity, FA salt) as a white solid. LCMS (Method E): Rt=0.478 min, [M+H]⁺=885.6. SFC: Rt=2.363, 2.541, 3.062 min. ¹H NMR (400 MHz, DMSO-d₆) δ=12.59 (s, 1H), 8.26 (d, J=8.0 Hz, 1H), 8.13 (s, 1H), 8.01-7.93 (m, 1H), 7.89 (t, J=7.2 Hz, 1H), 7.86-7.80 (m, 1H), 7.44 (d, J=2.4 Hz, 1H), 7.39-7.35 (m, 1H), 7.24 (t, J=9.2 Hz, 1H), 6.96-6.63 (m, 1H), 4.46-3.73 (m, 5H), 3.72-3.37 (m, 8H), 3.17 (d, J=8.8 Hz, 5H), 2.48-2.07 (m, 7H), 2.05-1.81 (m, 3H), 1.80-1.44 (m, 11H), 1.35 (s, 9H), 1.11 (br s, 4H), 1.01-0.75 (m, 2H). ¹⁹F NMR (377 MHz, DMSO-d₆) δ=−119.766.

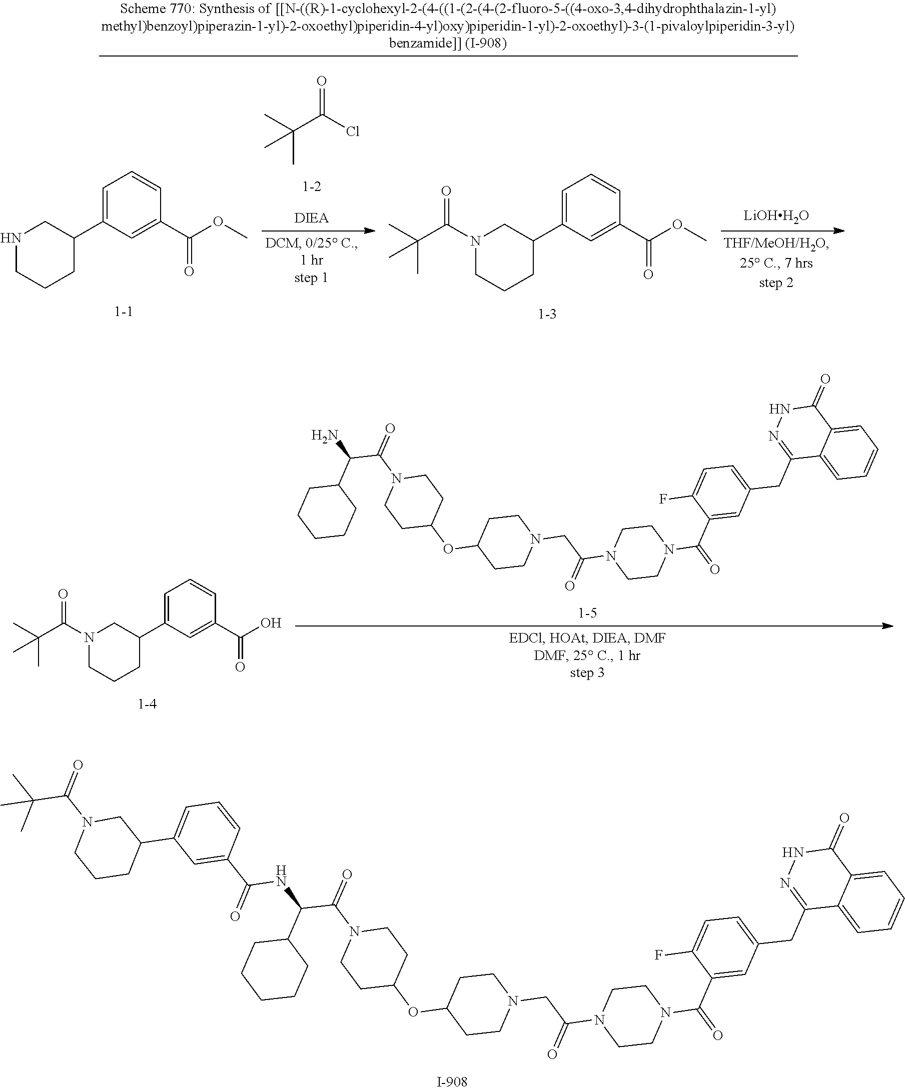

### Step 1: Synthesis of Intermediate 1-3

To a mixture of Intermediate 1-1 (3 g, 11.73 mmol, 1 eq, HCl salt) in DCM (30 mL) was added DIEA (4.55 g, 35.19 mmol, 6.13 mL, 3 eq) and Intermediate 1-2 (2.12 g, 17.60 mmol, 2.16 mL, 1.5 eq) at 0° C. The mixture was stirred at 25° C. for 1 hr. The reaction mixture was combined with another lot of material and was diluted with water (50 mL) and extracted with DCM (30 mL*3), the combined organic phase was dried over anhydrous Na₂SO₄ and concentrated to give the residue. The residue was purified by flash silica gel chromatography (ISCO; 80 g SepaFlash Silica Flash Column, Eluent of 0˜35% Ethyl acetate/Petroleum ether gradient @100 mL/min). The eluent was concentrated to give Intermediate 1-3 (3.3 g, 10.88 mmol, 92.72% yield, 100% purity) as yellow oil. LCMS (Method E): Retention time: 0.568 min, [M+H]⁺=304.3. SFC: Retention time: 0.691 min, 1.824 min. ¹H NMR (400 MHz, CHLOROFORM-d) δ=7.97-7.88 (m, 2H), 7.49-7.35 (m, 2H), 4.54 (d, J=8.8 Hz, 2H), 3.93 (s, 3H), 2.91-2.69 (m, 3H), 2.09 (d, J=13.2 Hz, 1H), 1.90-1.82 (m, 1H), 1.81-1.71 (m, 1H), 1.68-1.62 (m, 1H), 1.31 (s, 9H).

### Step 2: Synthesis of Intermediate 1-4

To the mixture of Intermediate 1-3 (3 g, 9.89 mmol, 1 eq) in THF (30 mL), MeOH (10 mL) and H₂O (10 mL) was added LiOH·H₂O (1.24 g, 29.66 mmol, 3 eq). The reaction mixture was stirred at 25° C. for 7 hrs. The reaction mixture was combined with another lot of material and HCl (1 N) was added to adjust the pH to 4, then the mixture was extracted with EA (30 mL*3), and the combined organic phase was dried over anhydrous Na₂SO₄ and concentrated to give Intermediate 1-3 (3 g, 10.37 mmol, 95% yield, 100% purity) as a white solid. LCMS (Method E): Retention time: 0.484 min, [M+H]⁺=290.1. SFC: Retention time: 1.726, 1.852 min. ¹H NMR (400 MHz, DMSO-d₆) δ=13.55-12.37 (m, 1H), 7.83-7.80 (m, 2H), 7.54-7.52 (m, 1H), 7.47-7.43 (m, 1H), 4.33-4.27 (m, 2H), 2.91-2.83 (m, 2H), 2.71-2.651 (m, 1H), 1.94 (d, J=10.0 Hz, 1H), 1.79-1.74 (m, 2H), 1.51-1.42 (m, 1H), 1.20 (s, 9H).

### Step 3: Synthesis of I-908

To a mixture of Intermediate 1-4 (207.68 mg, 717.70 μmol, 1 eq), Intermediate 1-5 (0.55 g, 717.70 μmol, 1 eq, HCl salt), EDCI (412.75 mg, 2.15 mmol, 3 eq) and NMM (362.96 mg, 3.59 mmol, 394.53 μL, 5 eq) in DMF (5.5 mL) was added HOAt (97.69 mg, 717.70 μmol, 100.40 μL, 1 eq) and then the mixture was stirred at 25° C. for 1 hr. The reaction mixture was quenched with water (20 mL) and extracted with EA (10 mL*3), the combined organic phase was washed by aqueous NaCl (20 mL*3) and dried by anhydrous Na₂SO₄, and concentrated to give crude product. The crude product was purified by reverse phase chromatography (105 g of XB—C18, 20-40 μm, 120 Å) Mobile phase: A for H₂O (0.1% FAv/v) and B for acetonitrile; Gradient: B30%-50% in 30 min; Flow rate: 100 ml/min; Column temperature: R.T. Wavelength: 220 nm/254 nm) and the eluent was then concentrated to remove organic solvents and then lyophilized to give product. I-908 (376.8 mg, 359.80 μmol, 50.13% yield, 100% purity, FA salt) as a white solid. LCMS (Method E): Retention time: 0.521 min, [M+H]⁺=1001.6. SFC: Retention time: 1.960 min, 2.362 min. ¹H NMR (400 MHz, DMSO-d6) δ=12.59 (s, 1H), 8.58-8.38 (m, 1H), 8.29-8.22 (m, 1H), 8.20-8.12 (m, 1H), 8.02-7.93 (m, 1H), 7.91-7.66 (m, 4H), 7.48-7.33 (m, 4H), 7.27-7.19 (m, 1H), 4.88-4.65 (m, 1H), 4.43-4.24 (m, 4H), 4.03-3.82 (m, 2H), 3.72-3.56 (m, 4H), 3.53-3.39 (m, 4H), 3.22-2.96 (m, 6H), 2.94-2.80 (m, 2H), 2.70-2.59 (m, 3H), 2.18-2.04 (m, 2H), 1.93-1.64 (m, 11H), 1.63-1.54 (m, 2H), 1.49-1.31 (m, 4H), 1.28-1.11 (m, 13H), 1.05-0.93 (m, 2H). ¹⁹F NMR (377 MHz, DMSO-d6) δ=−119.78 (br s, 1F).

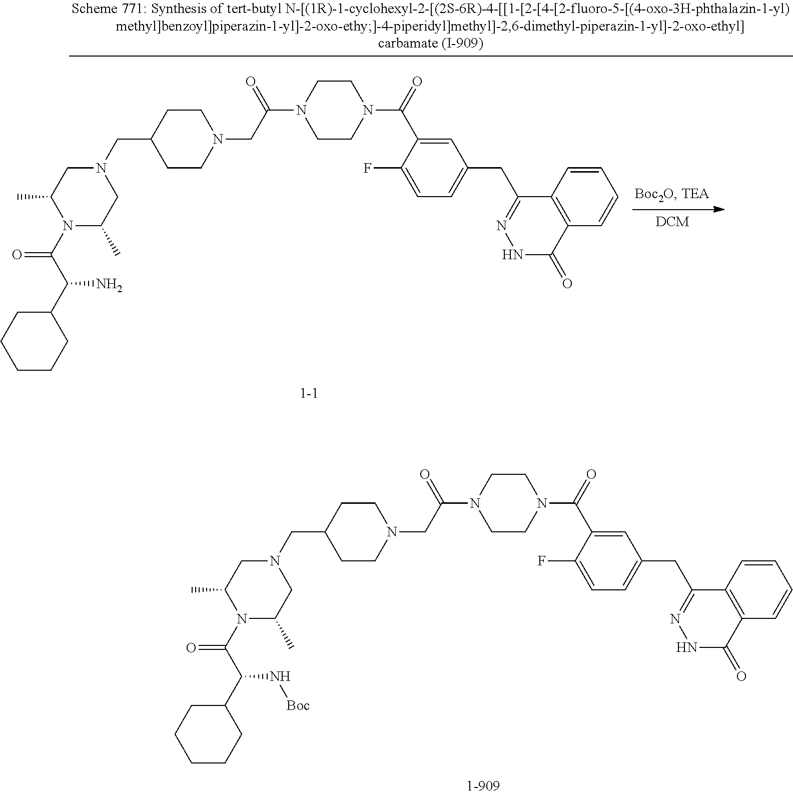 [SMILES: C[C@@H]1CN(CC2CCN(CC(=O)N3CCN(C(=O)c4cc(Cc5n[nH]c(=O)c6ccccc56)ccc4F)CC3)CC2)C[C@H](C)N1C(=O)[C@H](N)C1CCCCC1 ; C[C@@H]1CN(CC2CCN(CC(=O)N3CCN(C(=O)c4cc(Cc5n[nH]c(=O)c6ccccc56)ccc4F)CC3)CC2)C[C@H](C)N1C(=O)[C@H](NC(=O)OC(C)(C)C)C1CCCCC1]

To a solution of 4-[[3-[4-[2-[4-[[(3S,5R)-4-[(2R)-2-amino-2-cyclohexyl-acetyl]-3,5-dimethyl-piperazin-1-yl]methyl]-1-piperidyl]acetyl]piperazine-1-carbonyl]-4-fluoro-phenyl]methyl]-2H-phthalazin-1-one (30 mg, 39.63 μmol, 1 eq) in DCM (1 mL) was added Boc2O (19.03 mg, 87.19 μmol, 20.03 μL, 2.2 eq) and TEA (12.03 mg, 118.90 μmol, 16.55 μL, 3 eq). The mixture was stirred at 25° C. for 2 hr. The mixture was concentrated under reduced pressure to give a residue. The residue was purified by prep-HPLC (ISCO; 40 g SepaFlash C18 Column, Eluent of 0˜60%(0.1% FA) water/MeCN@40 mL/min). I-909 was obtained as a white solid (15 mg, 16.61 μmol, 41.91% yield, FA salt). LCMS (Method E): Rt=0.460 min, [M+H]⁺=857.6. SFC: Rt=0.743, 0.856 min, ee %=32.634%. ¹H NMR (400 MHz, DMSO-d6) δ=12.59 (s, 1H), 8.43-8.36 (m, 1H), 8.34-8.17 (m, 1H), 8.00-7.93 (m, 1H), 7.93-7.79 (m, 2H), 7.50-7.31 (m, 2H), 7.23 (t, J=9.0 Hz, 1H), 7.07-6.51 (m, 1H), 4.49-3.97 (m, 4H), 3.71-3.42 (m, 6H), 3.23-3.00 (m, 5H), 2.84-2.72 (m, 2H), 2.70-2.61 (m, 2H), 2.14-1.85 (m, 6H), 1.68 (br s, 5H), 1.62-1.42 (m, 4H), 1.40-1.28 (m, 12H), 1.19-0.80 (m, 10H). ¹⁹F NMR (377 MHz, DMSO-d6) δ=−119.71-−119.82 (m, 1F)

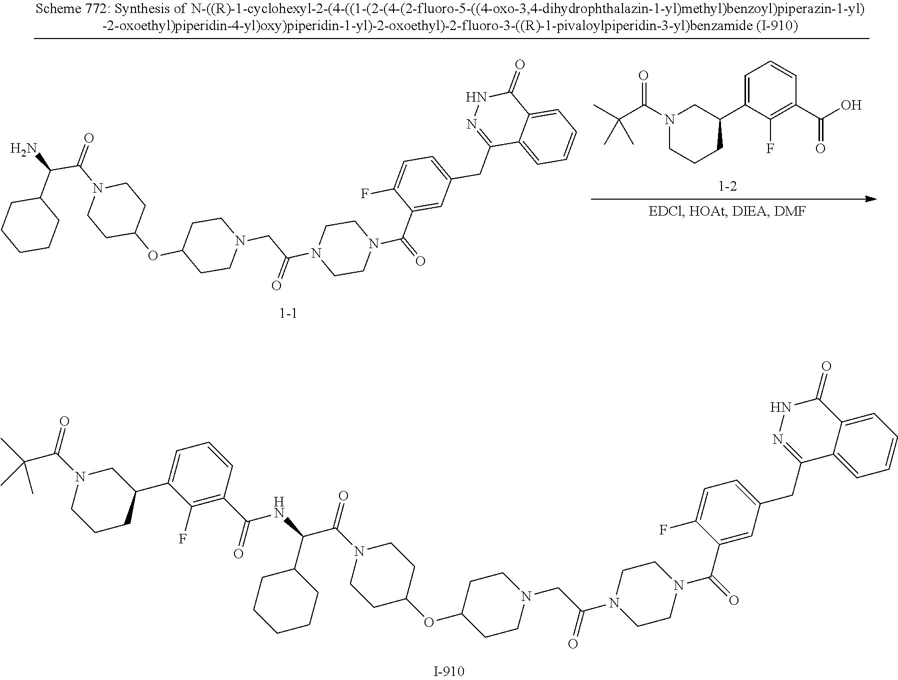

To a mixture of Intermediate 1-1 (0.5 g, 652.45 μmol, 1 eq, HCl salt) Intermediate 1-2 (200.54 mg, 652.45 μmol, 1 eq), EDCI (375.23 mg, 1.96 mmol, 3 eq) and NMM (527.95 mg, 5.22 mmol, 573.86 L, 8 eq) in DMF (5 mL) was added HOAt (88.81 mg, 652.45 μmol, 91.27 μL, 1 eq) and then the mixture was stirred at 25° C. for 1 h. The reaction mixture was combined with another lot of material for workup. The reaction mixture was quenched with water (20 mL) and extracted with EtOAc (10 mL*3), and the combined organic phase was washed by aqueous NaCl (20 mL*3) and dried by anhydrous Na₂SO₄, and concentrated to give crude product. The crude product was purified by reverse phase chromatography (105 g of XB—C18, 20-40 μm, 120 Å) Mobile phase: A for H₂O (0.1% FAv/v) and B for acetonitrile; Gradient: B30%-50% in 30 min; Flow rate: 100 ml/min; Column temperature: R.T. Wavelength: 220 nm/254 nm). Then the eluent was concentrated to remove organic solvents then lyophilized to give product. I-910 (294.74 mg, 287.54 μmol, 44.07% yield, 99.433% purity) as a white solid. LCMS (Method E): Retention time: 0.523 min, [M+H]⁺=1019.8. SFC: Retention time: 1.936 min. ¹H NMR (400 MHz, DMSO-d6) δ=12.59 (s, 1H), 8.50-8.35 (m, 1H), 8.26 (d, J=7.6 Hz, 1H), 8.16 (s, 1H), 8.00-7.94 (m, 1H), 7.92-7.79 (m, 2H), 7.53-7.34 (m, 4H), 7.29-7.17 (m, 2H), 4.83-4.72 (m, 1H), 4.44-4.26 (m, 4H), 4.01-3.81 (m, 2H), 3.72-3.54 (m, 5H), 3.52-3.43 (m, 4H), 3.22-2.97 (m, 7H), 2.95-2.80 (m, 3H), 2.71-2.62 (m, 2H), 2.20-2.06 (m, 2H), 1.92-1.65 (m, 11H), 1.60 (d, J=8.4 Hz, 2H), 1.51-1.34 (m, 4H), 1.21 (s, 8H), 1.17-1.10 (m, 2H), 1.09-0.98 (m, 2H). ¹⁹F NMR (376 MHz, DMSO-d6) δ=−119.78 (br s, 1F), −120.01 (br d, J=9.0 Hz, 1F).

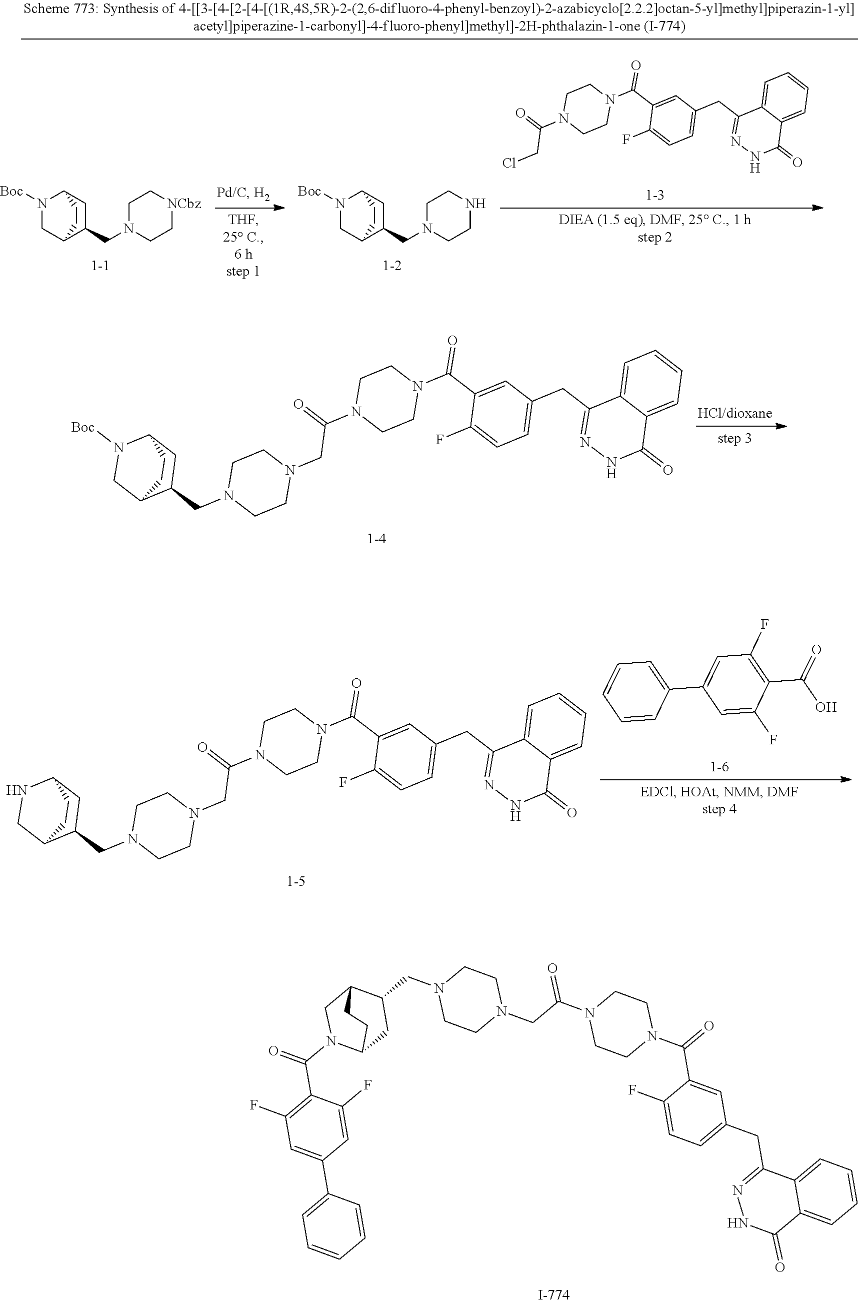

### Step 1: Synthesis of Intermediate 1-2

To a solution of Intermediate 1-1 (490 mg, 1.10 mmol, 1 eq) in THF (5 mL) was added Pd/C (200 mg, 187.93 μmol, 10.30 μL, 10% purity, 0.17 eq) under N₂, and the reaction mixture was purged with H₂ three times, and the reaction mixture was stirred at 25° C. for 6 h under H₂ (15 psi). The suspension was filtered through a pad of Celite and the filter cake was washed with THF (5 mL*5). The combined filtrates were concentrated to dryness to give the crude product. The crude product was used in the next step directly. Intermediate 1-1 (350 mg, crude) was obtained as an-off white solid. LCMS (Method E): Rt=0.349 min, [M+H]⁺=310.2. ¹H NMR (400 MHz, DMSO-d6) δ=3.80 (d, J=18.4 Hz, 1H), 3.11-3.04 (m, 1H), 2.72-2.60 (m, 4H), 2.32-2.14 (m, 5H), 2.11-2.04 (m, 1H), 1.93 (br s, 1H), 1.83-1.77 (m, 1H), 1.75-1.64 (m, 2H), 1.62-1.51 (m, 3H), 1.38 (d, J=5.2 Hz, 9H), 1.20-1.15 (m, 1H).

### Step 2: Synthesis of Intermediate 1-4

To a solution of intermediate 1-2 (350 mg, 1.13 mmol, 1 eq) and intermediate 1-3 (500.91 mg, 1.13 mmol, 1 eq) in DMF (3 mL) was added DIEA (219.27 mg, 1.70 mmol, 295.51 μL, 1.5 eq), and the reaction mixture was stirred at 25° C. for 1 h. To the reaction mixture was added with water (10 mL) and it was extracted with EA (5 mL*3), washed by brine (10 mL*2), the combined organic phase were dried over Na₂SO₄, filtered and concentrated under reduced pressure to give a residue. The crude product was purified by reverse phase chromatography twice (100 g of XB—C18, 20-40 μm, 120 Å) Mobile phase: A for H₂O (0.1% NH₃H₂O v/v) and B for acetonitrile; Gradient: B40%-50% in 10 min; Flow rate: 100 ml/min; Column temperature: R.T. Wavelength: 220 nm/254 nm), then concentrated to remove organic solvents and then extracted with EA (10 mL*3), and the collected organic phase was dried over Na₂SO₄, filtered and concentrated under reduced pressure to give intermediate 1-4 (640 mg, 894.04 μmol, 79.04% yield) as a white solid. LCMS (Method G): Rt=0.612 min, [M+H]⁺=716.4. SFC: Rt=4.517 min. ¹H NMR (400 MHz, DMSO-d6) δ=12.59 (s, 1H), 8.26 (d, J=7.6 Hz, 1H), 8.01-7.93 (m, 1H), 7.93-7.79 (m, 2H), 7.49-7.32 (m, 2H), 7.23 (t, J=8.8 Hz, 1H), 4.33 (s, 2H), 3.80 (d, J=18.8 Hz, 1H), 3.67-3.42 (m, 5H), 3.36 (d, J=3.8 Hz, 2H), 3.22-3.01 (m, 5H), 2.46-2.05 (m, 10H), 1.95-1.84 (m, 1H), 1.82-1.65 (m, 3H), 1.61-1.50 (m, 3H), 1.38 (d, J=4.8 Hz, 9H), 1.23 (br s, 1H). ¹⁹F NMR (377 MHz, DMSO-d6) δ=−119.78 (s, 1F).

### Step 3: Synthesis of Intermediate 1-5

To a solution of intermediate 1-4 (290 mg, 405.11 μmol, 1 eq) in DCM (1 mL) was added HCl/dioxane (2 mL) (2 N), and the reaction mixture was stirred at 25° C. for 2 h. The reaction mixture was concentrated to give crude product. The crude product was used in the next step directly without any other purification. Intermediate 1-5 (300 mg, crude, HCl salt) was obtained as a white solid. LCMS (Method E): Rt=0.371 min, [M+H]⁺=616.4. SFC: Rt=1.640 min. ¹H NMR (400 MHz, DMSO-d6) δ=12.61 (d, J=4.0 Hz, 1H), 9.31-8.92 (m, 2H), 8.26 (d, J=7.6 Hz, 1H), 8.02-7.79 (m, 3H), 7.51-7.43 (m, 1H), 7.37-7.27 (m, 1H), 7.25 (t, J=9.2 Hz, 1H), 4.58-4.21 (m, 4H), 3.81-3.58 (m, 11H), 3.41-3.15 (m, 11H), 2.95 (d, J=6.8 Hz, 1H), 2.36-2.23 (m, 1H), 2.17 (br s, 1H), 1.97 (d, J=11.2 Hz, 2H), 1.74-1.57 (m, 4H). ¹⁹F NMR (377 MHz, DMSO-d6) δ=−119.72 (s, 1F).

### Step 4: Synthesis of I-774

To Intermediate 1-6 (53.86 mg, 229.99 μmol, 1 eq) in DMF (1.5 mL) was added EDCI (132.27 mg, 689.97 μmol, 3 eq), HOAt (46.96 mg, 344.99 μmol, 48.26 μL, 1.5 eq), NMM (116.31 mg, 1.15 mmol, 126.43 μL, 5 eq) and intermediate 1-5 (150 mg, crude, HCl salt) and the reaction mixture was stirred at 25° C. for 1 h. The reaction mixture was combined with another lot of material. The mixture was added to water (10 mL) and extracted with EA (5 mL*3), the combined organic phase was washed by brine (10 mL), then dried over Na₂SO₄, filtered and concentrated under reduced pressure to give the residue. The crude product was purified by reverse phase chromatography twice (100 g of XB—C18, 20-40 μm, 120 Å) Mobile phase: A for H₂O (0.1% NH3·H2Ov/v) and B for acetonitrile; Gradient: B50%-60% in 10 min; Flow rate: 100 ml/min; Column temperature: R.T. Wavelength: 220 nm/254 nm), and then concentrated to remove organic solvents and lyophilized to give product. I-774 (188 mg, 222.93 μmol, 96.93% yield, 98.65% purity) was obtained as a white solid. LCMS (Method G): Rt=1.897 min, [M+H]⁺=832.5. SFC: Rt=1.674 min, 1.801 min. ¹H NMR (400 MHz, DMSO-d6) δ=12.59 (s, 1H), 8.26 (d, J=7.6 Hz, 1H), 8.01-7.94 (m, 1H), 7.93-7.80 (m, 2H), 7.77 (d, J=7.2 Hz, 2H), 7.62-7.54 (m, 2H), 7.54-7.41 (m, 4H), 7.37 (d, J=6.0 Hz, 1H), 7.28-7.20 (m, 1H), 4.45-4.30 (m, 2H), 3.70-3.33 (m, 9H), 3.21-3.06 (m, 4H), 2.47-2.10 (m, 10H), 2.08-1.88 (m, 2H), 1.86-1.59 (m, 5H), 1.35-1.20 (m, 1H). ¹⁹F NMR (377 MHz, DMSO-d6) δ=−113.68-−115.40 (m, 2F), −119.78 (s, 1F).

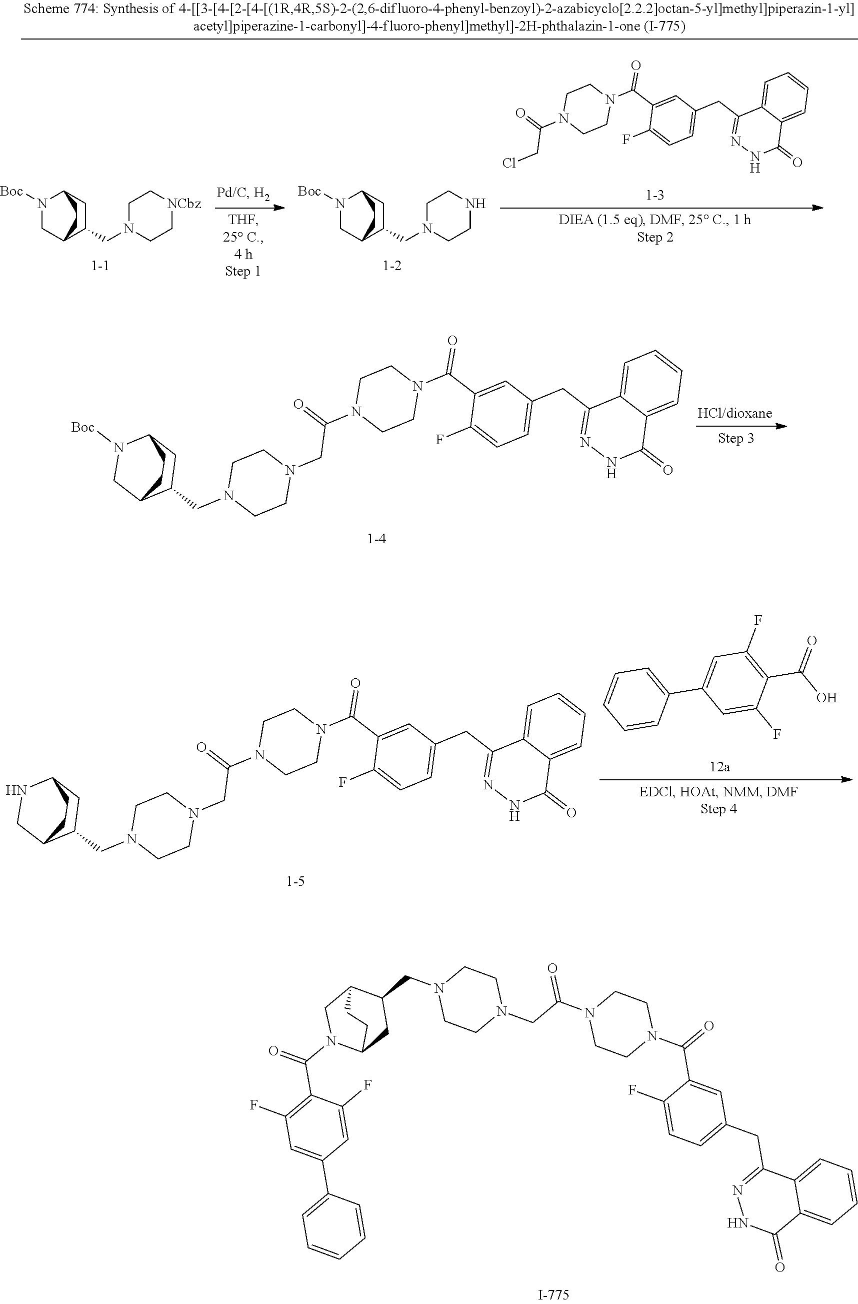

### Step 1: Synthesis of Intermediate 1-2

To a solution of Intermediate 1-1 (600 mg, 1.35 mmol, 1 eq) in THF (6 mL) was added Pd/C (300 mg, 281.90 μmol, 10.30 μL, 10% purity, 2.08e−1 eq) under N₂, and the reaction mixture was purged with H₂ three times, and the reaction mixture was stirred at 25° C. for 6 h under H₂ (15 psi). The suspension was filtered through a pad of Celite and the filter cake was washed with THF (5 mL*5). The combined filtrates were concentrated to dryness to give crude product. The crude product was used in the next step directly. Intermediate 1-2 (400 mg, crude) was obtained as an-off white solid. LCMS (Method E): Rt=0.350 min, [M+H]⁺=310.1. ¹H NMR (400 MHz, DMSO-d6) δ=3.80 (d, J=18.5 Hz, 1H), 3.13-3.01 (m, 1H), 2.71-2.60 (m, 4H), 2.34-2.13 (m, 5H), 2.11-2.03 (m, 1H), 1.99-1.87 (m, 1H), 1.77 (d, J=3.4 Hz, 1H), 1.75-1.63 (m, 2H), 1.62-1.50 (m, 3H), 1.42-1.33 (m, 9H), 1.25-1.15 (m, 1H).

### Step 2: Synthesis of Intermediate 1-4

To a solution of intermediate 1-2 (350 mg, 1.13 mmol, 1 eq) and intermediate 1-3 (500.91 mg, 1.13 mmol, 1 eq) in DMF (3.5 mL) was added DIEA (219.27 mg, 1.70 mmol, 295.51 μL, 1.5 eq) and the reaction mixture was stirred at 25° C. for 1 h. The mixture was added to water (10 mL) and extracted with EA (5 mL*3), dried over Na₂SO₄, filtered and concentrated under reduced pressure to give the residue. The crude product was purified by reverse phase chromatography twice (100 g of XB—C18, 20-40Etm, 120 Å) Mobile phase: A for H₂O (0.1% NH3·H2Ov/v) and B for acetonitrile; Gradient: B40%-50% in 10 min; Flow rate: 100 ml/min; Column temperature: R.T. Wavelength: 220 nm/254 nm) and then concentrated to remove organic solvents and then extracted with EA (10 mL*3). The collected organic phases were dried over Na₂SO₄, filtered and concentrated under reduced pressure to give the product intermediate 1-4 (750 mg, 1.05 mmol, 92.63% yield) as a white solid. LCMS (Method G): Rt=0.608 min, [M+H]⁺=716.4. SFC: Rt=5.031 min ¹H NMR (400 MHz, DMSO-d6) δ=12.59 (s, 1H), 8.26 (d, J=8.0 Hz, 1H), 8.02-7.78 (m, 3H), 7.49-7.33 (m, 2H), 7.23 (t, J=9.2 Hz, 1H), 4.33 (s, 2H), 3.87-3.74 (m, 1H), 3.68-3.39 (m, 5H), 3.38-3.33 (m, 1H), 3.30-3.22 (m, 1H), 3.20-3.03 (m, 5H), 2.44-2.14 (m, 10H), 1.94-1.86 (m, 1H), 1.80-1.66 (m, 3H), 1.62-1.51 (m, 3H), 1.38 (d, J=5.2 Hz, 9H), 1.22-1.19 (m, 1H). ¹⁹F NMR (377 MHz, DMSO-d6) δ=−119.79 (s, 1F).

### Step 3: Synthesis of Intermediate 1-5

To a solution of intermediate 1-4 (390 mg, 544.80 μmol, 1 eq) in DCM (1.5 mL) was added HCl/dioxane (2.5 mL) (2 N), and the reaction mixture was stirred at 25° C. for 2 h. The reaction mixture was concentrated to give crude product. The crude product was used in the next step directly without any other purification. intermediate 1-5 (400 mg, crude, HCl salt) was obtained as a white solid. LCMS (Method E): Rt=0.372 min, [M+H]⁺=616.4. SFC: Rt=1.628 min. ¹H NMR (400 MHz, DMSO-d6) δ=12.61 (d, J=4.4 Hz, 1H), 9.33-8.81 (m, 2H), 8.26 (d, J=7.6 Hz, 1H), 8.00-7.80 (m, 3H), 7.51-7.43 (m, 1H), 7.36-7.28 (m, 1H), 7.25 (t, J=9.0 Hz, 1H), 4.34 (s, 4H), 3.74-3.58 (m, 11H), 3.42-3.19 (m, 11H), 2.99-2.90 (m, 1H), 2.35-2.24 (m, 1H), 2.17 (br s, 1H), 2.03-1.89 (m, 2H), 1.73-1.59 (m, 4H). ¹⁹F NMR (377 MHz, DMSO-d6) δ=−119.72 (s, 1F).

### Step 4: Synthesis of I-775

To Intermediate 1-6 (53.86 mg, 229.99 μmol, 1 eq) in DMF (1.5 mL) was added EDCI (132.27 mg, 689.97 μmol, 3 eq), HOAt (46.96 mg, 344.99 μmol, 48.26 μL, 1.5 eq), NMM (116.32 mg, 1.15 mmol, 126.43 μL, 5 eq) and intermediate 1-5 (150 mg, 229.99 μmol, 1 eq, HCl salt). the reaction mixture was stirred at 25° C. for 1 h. The reaction mixture was combined with another lot of material. To the mixture was added water (10 mL) and it was extracted with EA (5 mL*3), and the combined organic phase was washed by brine (10 mL), then dried over Na₂SO₄, filtered and concentrated under reduced pressure to give the residue. The crude product was purified by reverse phase chromatography twice (100 g of XB—C18, 20-40 μm, 120 Å) Mobile phase: A for H₂O (0.1% NH3·H2Ov/v) and B for acetonitrile; Gradient: B50%-60% in 20 min; Flow rate: 100 ml/min; Column temperature: R.T. Wavelength: 220 nm/254 nm), and then concentrated to remove organic solvents and lyophilized to give I-775 (160 mg, 191.23 μmol, 83.15% yield, 99.43% purity) as a white solid. LCMS (Method G): Rt=1.888 min, [M+H]⁺=832.5. SFC: Rt=1.631 min, 1.859 min. ¹H NMR (400 MHz, DMSO-d6) δ=12.59 (s, 1H), 8.26 (d, J=7.6 Hz, 1H), 8.00-7.94 (m, 1H), 7.92-7.80 (m, 2H), 7.80-7.74 (m, 2H), 7.62-7.54 (m, 2H), 7.53-7.42 (m, 4H), 7.38 (br s, 1H), 7.28-7.19 (m, 1H), 4.33 (s, 2H), 3.68-3.34 (m, 9H), 3.22-3.05 (m, 4H), 2.48-2.09 (m, 10H), 2.05-1.89 (m, 2H), 1.87-1.58 (m, 5H), 1.35-1.21 (m, 1H). ¹⁹F NMR (377 MHz, DMSO-d6) δ=−113.64-−115.14 (m, 2F), −119.78 (s, 1F).

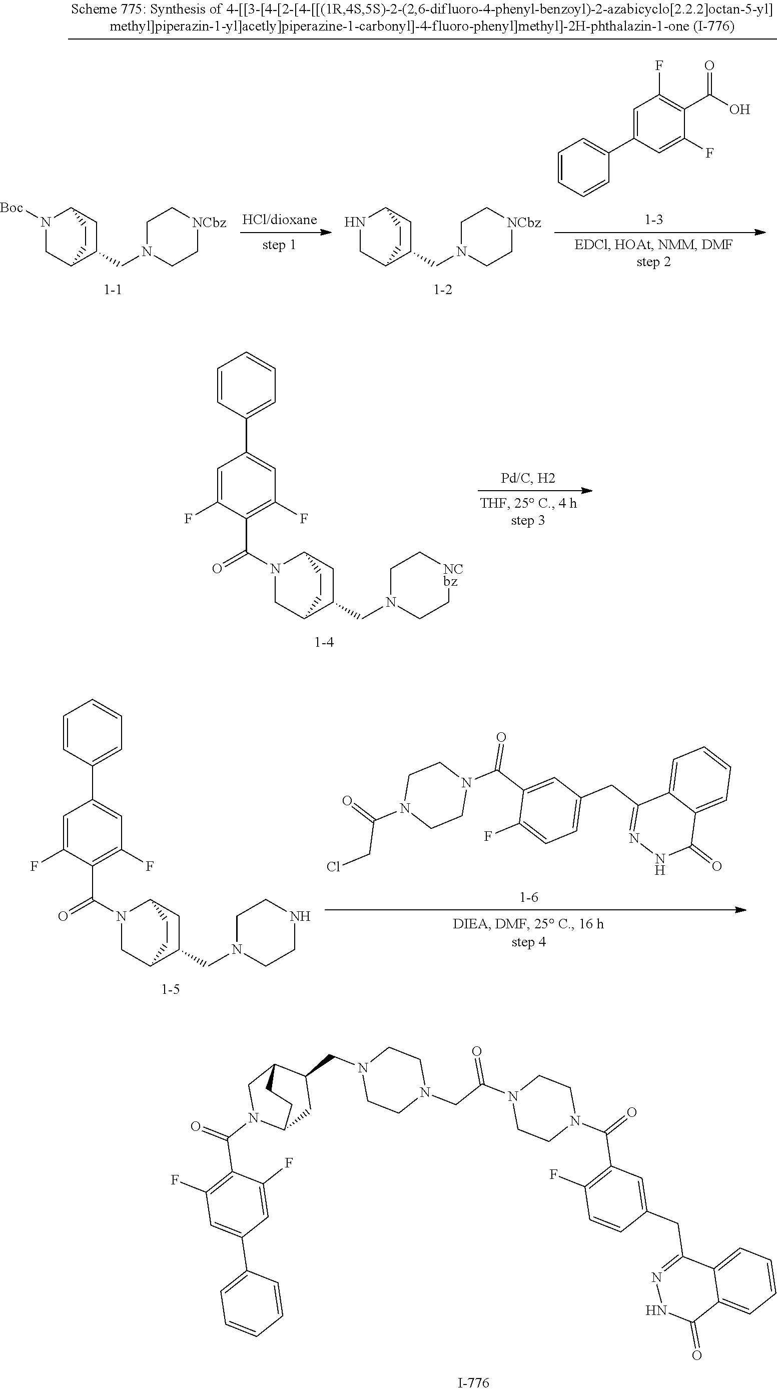

### Step 1: Synthesis of Intermediate 1-2

To a solution of Intermediate 1-1 (420.00 mg, 946.85 μmol, 1 eq) in DCM (3 mL) was added HCl/dioxane (3 mL) (2 M), and the reaction mixture was stirred at 25° C. for 1 h. The reaction mixture was concentrated to give crude product intermediate 1-2 (400 mg, crude, HCl salt) as a white solid. NMR analysis indicated the product and de-cbz by-product were present in a 1:1 ratio. LCMS (Method E): Rt=0.3944 min, [M+H]⁺=344.2. ¹H NMR (400 MHz, DMSO-d6) δ=11.16 (d, J=1.4 Hz, 1H), 9.22 (d, J=4.0 Hz, 1H), 7.48-7.27 (m, 3H), 5.12 (s, 1H), 4.05 (d, J=14.0 Hz, 1H), 3.77 (br s, 3H), 3.55-3.40 (m, 2H), 3.39-2.94 (m, 4H), 2.42-2.18 (m, 1H), 2.17-2.06 (m, 1H), 1.96-1.73 (m, 1H), 1.64-1.44 (m, 1H), 1.34-1.30 (m, 1H).

### Step 2: Synthesis of Intermediate 1-4

To a solution of intermediate 1-3 (271.23 mg, 1.16 mmol, 1.1 eq) in DMF (4 mL) was added EDCI (605.49 mg, 3.16 mmol, 3 eq), HOAt (214.96 mg, 1.58 mmol, 220.92 μL, 1.5 eq), NMM (532.46 mg, 5.26 mmol, 578.76 μL, 5 eq), and intermediate 1-2 (400.00 mg, crude, HCl salt), and the reaction mixture was stirred at 25° C. for 1 h. The reaction mixture was quenched by water (10 mL), and extracted by EA (5 mL*3). The combined organics were washed by brine (10 mL*2). The organic phase was dried by anhydrous Na₂SO₄, filtered and the concentrated to afford the crude. The crude product was purified by reverse phase chromatography (80 g of XB—C18, 20-40 μm, 120 Å) Mobile phase: A for H₂O (0.1% NH₃·H₂Ov/v) and B for acetonitrile; Gradient: B40%-50% in 10 min; Flow rate: 100 ml/min; Column temperature: R.T. Wavelength: 220 nm/254 nm), and then concentrated to remove organic solvents and lyophilized to give intermediate 1-4 (400 mg, 714.74 μmol, 67.89% yield) as a white solid. LCMS (Method E): Rt=0.492 min, [M+H]⁺=560.3. SFC: Rt=1.992 min, 2.110 min, ¹H NMR (400 MHz, DMSO-d6) δ=7.81-7.73 (m, 2H), 7.60-7.54 (m, 2H), 7.53-7.43 (m, 3H), 7.41-7.28 (m, 5H), 5.11-5.01 (m, 2H), 3.52 (br s, 2H), 3.43-3.37 (m, 4H), 2.43-2.18 (m, 7H), 2.14-2.04 (m, 2H), 1.93-1.42 (m, 6H). ¹⁹F NMR (377 MHz, DMSO-d6) δ=−114.11 (br dd, J=4.5, 20.2 Hz, 1F).

### Step 3: Synthesis of Intermediate 1-5

To a solution of intermediate 1-4 (400 mg, 714.74 μmol, 1 eq) in THF (4 mL) was added Pd/C (200 mg, 187.93 μmol, 10.30 μL, 10% purity, 2.63e−1 eq) under N₂, and the reaction mixture was purged with H₂ three times, and the reaction mixture was stirred at 25° C. for 6 h under H₂ (15 psi). The suspension was filtered through a pad of Celite and the filter cake was washed with THF (5 mL*5). The combined filtrates were concentrated to dryness to give crude product. The crude product was used in the next step directly. intermediate 1-5 (300 mg, crude) was obtained as a white solid. LCMS (Method E): Rt=0.416 min, [M+H]⁺=426.2. ¹H NMR (400 MHz, DMSO-d6) δ=7.77 (d, J=7.2 Hz, 2H), 7.61-7.54 (m, 2H), 7.53-7.41 (m, 3H), 3.51 (br s, 2H), 3.40 (br s, 2H), 2.66 (t, J=4.4 Hz, 4H), 2.36-2.14 (m, 6H), 2.11-1.95 (m, 2H), 1.90-1.81 (m, 1H), 1.75-1.59 (m, 3H), 1.58-1.46 (m, 1H), 1.20-1.10 (m, 1H). ¹⁹F NMR (377 MHz, DMSO-d6) δ=−114.14 (d, J=9.2 Hz, 1F).

### Step 4: Synthesis of I-776

To Intermediate 1-5 (250 mg, crude) and Intermediate 1-6 (260.20 mg, 587.53 μmol, 1 eq) in DMF (2.5 mL) was added DIEA (113.90 mg, 881.29 μmol, 153.50 μL, 1.5 eq), and the reaction mixture was stirred at 25° C. for 1 h. The reaction mixture was combined with another lot of material. To the mixture was added with water (10 mL) and it was extracted with EA (5 mL*3), dried over Na₂SO₄, filtered and concentrated under reduced pressure to give the residue. The crude product was purified by reverse phase chromatography twice (100 g of XB—C18, 20-40 μm, 120 Å) Mobile phase: A for H₂O (0.1% NH₃·H₂Ov/v) and B for acetonitrile; Gradient: B40%-50% in 30 min; Flow rate: 100 ml/min; Column temperature: R.T. Wavelength: 220 nm/254 nm) and then concentrated to remove organic solvents and lyophilized to give product. I-776 (400 mg, 480.81 μmol, 81.84% yield, 98.99% purity) was obtained as a white solid. LCMS (Method G): Rt=0.651 min, [M+H]⁺=832.5. SFC: Rt=1.891 min, 2.603 min. ¹H NMR (400 MHz, DMSO-d6) δ=12.59 (s, 1H), 8.26 (d, J=7.6 Hz, 1H), 8.00-7.94 (m, 1H), 7.92-7.80 (m, 2H), 7.77 (d, J=7.2 Hz, 2H), 7.60-7.53 (m, 2H), 7.53-7.41 (m, 4H), 7.37 (t, J=6.4 Hz, 1H), 7.23 (t, J=8.8 Hz, 1H), 4.33 (s, 2H), 3.70-3.33 (m, 9H), 3.23-3.05 (m, 4H), 2.49-2.12 (m, 10H), 2.11-1.83 (m, 3H), 1.80-1.43 (m, 4H), 1.26-1.08 (m, 1H). ¹⁹F NMR (377 MHz, DMSO-d6) δ=−114.12 (br d, J=9.0 Hz, 2F), −119.78 (s, 1F).

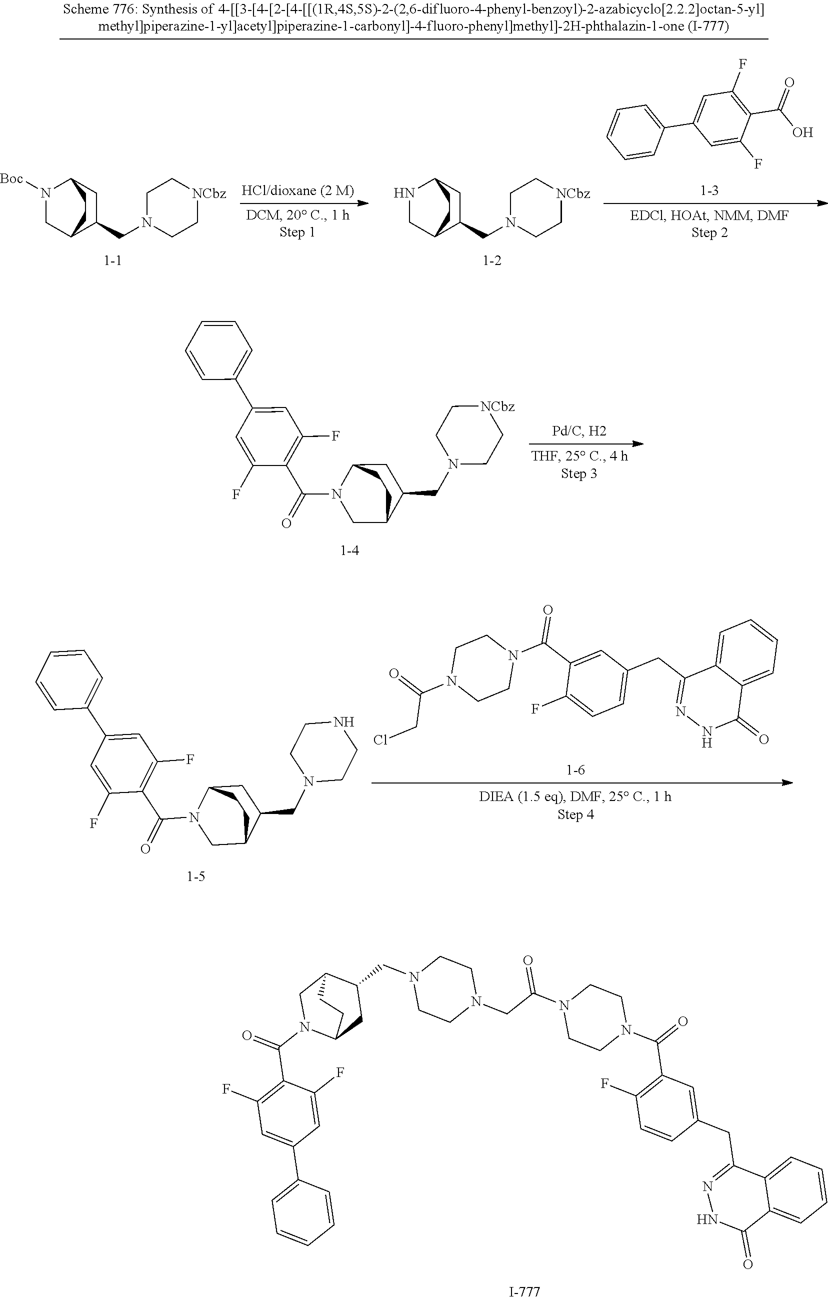

### Step 1: Synthesis of Intermediate 1-2

To a solution of Intermediate 1-1 (620.00 mg, 1.40 mmol, 1 eq) in DCM (4 mL) was added HCl/dioxane (4 mL, 2M), and the reaction mixture was stirred at 25° C. for 1 h. The crude product was used in the next step directly without any other purification. Intermediate 1-2 (620 mg, crude, HCl salt) was obtained as a yellow solid. LCMS (Method E): Rt=0.350 min, [M+H]⁺=344.2. ¹HNMR (400 MHz, DMSO-d6) δ=11.08 (d, J=2.8 Hz, 1H), 9.36-8.72 (m, 2H), 7.52-7.21 (m, 3H), 5.12 (s, 1H), 4.05 (d, J=14.0 Hz, 1H), 3.57-3.40 (m, 4H), 3.39-2.82 (m, 7H), 2.41-2.29 (m, 1H), 2.29-2.16 (m, 1H), 2.09 (br s, 1H), 1.99-1.70 (m, 3H), 1.66-1.45 (m, 2H), 1.39-1.21 (m, 1H).

### Step 2: Synthesis of Intermediate 1-4

To a solution of intermediate 1-3 (216.99 mg, 926.50 μmol, 1.1 eq) in DMF (3.2 mL) was added EDCI (484.40 mg, 2.53 mmol, 3 eq), HOAt (171.96 mg, 1.26 mmol, 176.74 μL, 1.5 eq), NMM (425.97 mg, 4.21 mmol, 463.01 μL, 5 eq), intermediate 1-2 (320 mg, 842.28 μmol, 1 eq, HCl salt) and the reaction mixture was stirred at 25° C. for 1 h. The reaction mixture was quenched by water (10 mL), and extracted by EA (5 mL*3). The organics were washed by brine (10 mL*2). The organic phase was dried by anhydrous Na₂SO₄, filtered and the concentrated to afford the crude. The crude product was purified by reverse phase chromatogrpahy (80 g of XB—C18, 20-40 μm, 120 Å) Mobile phase: A for H₂O (0.1% NH₃·H₂Ov/v) and B for acetonitrile; Gradient: B40%-50% in 10 min; Flow rate: 100 ml/min; Column temperature: R.T. Wavelength: 220 nm/254 nm), then concentrated to remove organic solvents and lyophilized to give product. Intermediate 1-4 (250 mg, 446.71 μmol, 53.04% yield) was obtained as a white solid. LCMS (Method E): Rt=0.493 min, [M+H]⁺=560.3. SFC: Rt=1.916 min, 2.400 min. ¹H NMR (400 MHz, DMSO-d6) δ=7.77 (d, J=7.2 Hz, 2H), 7.63-7.42 (m, 5H), 7.41-7.27 (m, 5H), 5.13-4.98 (m, 2H), 3.54-3.36 (m, 6H), 2.45-2.19 (m, 7H), 2.14-1.99 (m, 2H), 1.97-1.44 (m, 6H). ¹⁹F NMR (377 MHz, DMSO-d6) δ=−113.49-−114.72 (m, 1F).

### Step 3: Synthesis of Intermediate 1-5

To a solution of intermediate 1-4 (250.00 mg, 446.71 μmol, 1 eq) in THF (2.5 mL) was added Pd/C (100 mg, 93.97 μmol, 10% purity, 0.21 eq) under N₂, and the reaction mixture was purged with H₂ three times, and the reaction mixture was stirred at 25° C. for 6 h under H₂ (15 psi). The suspension was filtered through a pad of Celite and the filter cake was washed with THF (5 mL*5). The combined filtrates were concentrated to dryness to give crude product. The crude product was used in the next step directly. intermediate 1-5 (185 mg, crude) was obtained as a white solid. LCMS (Method E): Rt=0.416 min, [M+H]⁺=426.2. ¹H NMR (400 MHz, DMSO-d6) δ=7.80-7.74 (m, 2H), 7.61-7.54 (m, 2H), 7.53-7.42 (m, 3H), 3.54-3.46 (m, 2H), 3.40 (br s, 2H), 2.73-2.60 (m, 4H), 2.37-2.16 (m, 6H), 2.11-1.96 (m, 2H), 1.90-1.81 (m, 1H), 1.75-1.60 (m, 3H), 1.58-1.47 (m, 1H), 1.20-1.11 (m, 1H). ¹⁹F NMR (377 MHz, DMSO-d6) δ=−110.25-−117.62 (m, 1F).

### Step 4: Synthesis of I-777

To Intermediate 1-5 (185.00 mg, crude) and Intermediate 1-6 (192.55 mg, 434.77 μmol, 1 eq) in DMF (2 mL) was added DIEA (84.29 mg, 652.15 μmol, 113.59 μL, 1.5 eq), and the reaction mixture was stirred at 25° C. for 1 h. To the mixture was added with water (10 mL) and it was extracted with EA (5 mL*3), dried over Na₂SO₄, filtered and concentrated under reduced pressure to give the residue. The crude product was purified by reverse phase chromatography twice (100 g of XB—C18, 20-40 μm, 120 Å) Mobile phase: A for H₂O (0.1% NH₃·H₂Ov/v) and B for acetonitrile; Gradient: B40%-50% in 10 min; Flow rate: 100 ml/min; Column temperature: R.T. Wavelength: 220 nm/254 nm), and then concentrated to remove organic solvents and lyophilized to give I-777 (300 mg, 357.04 μmol, 82.12% yield, 99.01% purity) as a white solid. LCMS (Method G): Rt=0.651 min, [M+H]⁺=832.5. SFC: Rt=1.914 min, 2.179 min, ¹H NMR (400 MHz, DMSO-d6) δ=12.59 (s, 1H), 8.26 (d, J=7.6 Hz, 1H), 8.00-7.94 (m, 1H), 7.92-7.80 (m, 2H), 7.77 (d, J=7.6 Hz, 2H), 7.61-7.53 (m, 2H), 7.52-7.42 (m, 4H), 7.37 (t, J=6.4 Hz, 1H), 7.23 (t, J=8.8 Hz, 1H), 4.33 (s, 2H), 3.70-3.33 (m, 9H), 3.31-3.04 (m, 5H), 2.46-2.13 (m, 9H), 2.11-1.93 (m, 2H), 1.91-1.81 (m, 1H), 1.79-1.59 (m, 3H), 1.58-1.44 (m, 1H), 1.26-1.09 (m, 1H). ¹⁹F NMR (377 MHz, DMSO-d6) δ=−114.12 (br d, J=9.0 Hz, 2F), −119.78 (s, 1F).

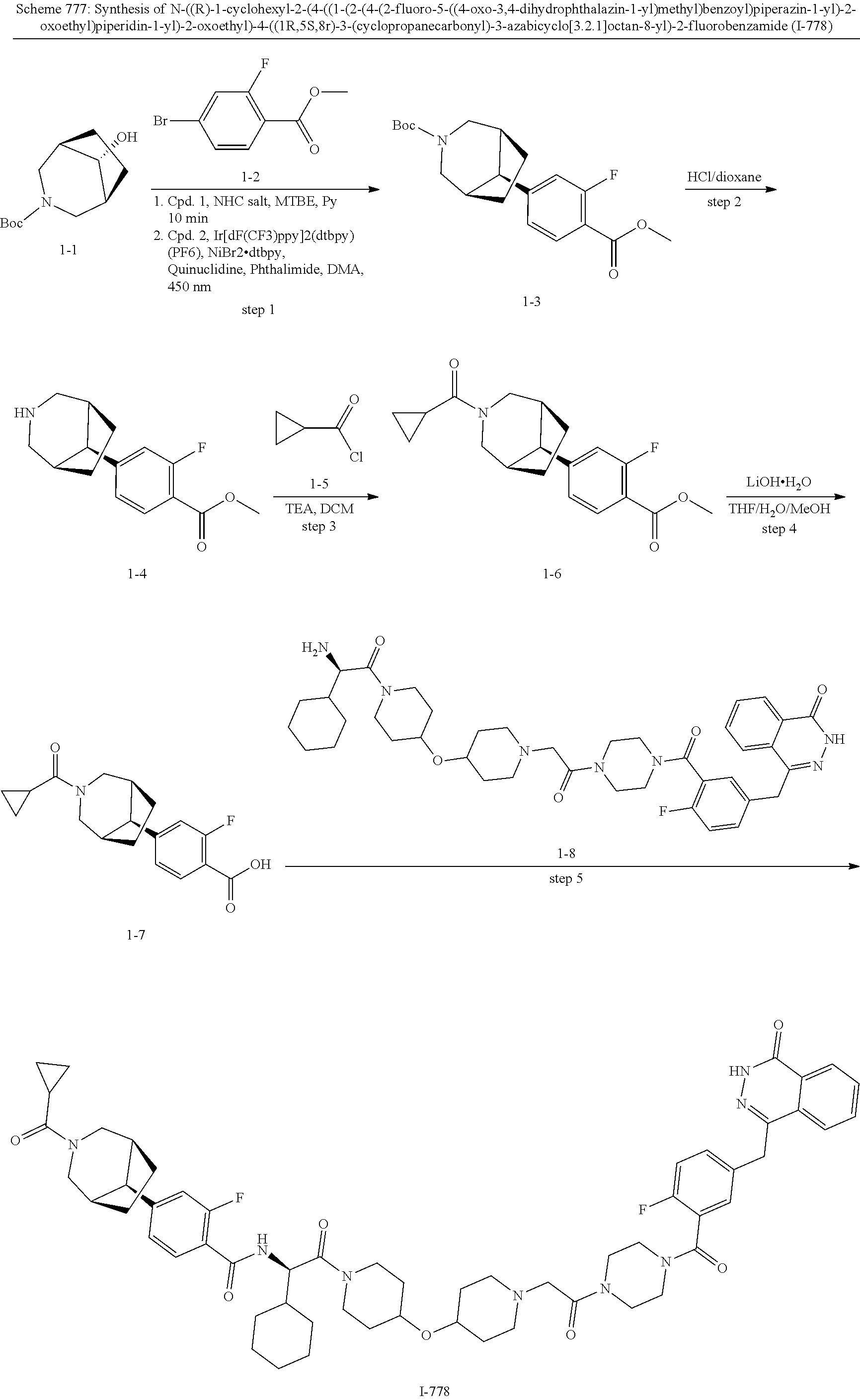

### Step 1: Synthesis of Intermediate 1-3

An oven-dried 8 mL vial was charged with intermediate 1-1 (93.76 mg, 412.50 μmol, 1.65 eq) and NHC-salt (158.10 mg, 400.00 μmol, 1.6 eq). After the vial was vacuumed and refilled with nitrogen gas twice, MTBE (2 mL) was added and the reaction was stirred at 25° C. for 5 min. Then, a pyridine solution (31.64 mg, 400.00 μmol, 32.29 μL, 1.6 eq) in MTBE (0.5 mL) was added dropwise at 25° C. over the course of 2 min. The resulting solution stirred at 25° C. for 10 min. A white solid precipitated out during this time. Another oven-dried 15 mL vial was charged with bis[3,5-difluoro-2-[5-(trifluoromethyl)-2-pyridyl]phenyl]iridium (1+); 4-tert-butyl-2-(4-tert-butyl-2-pyridyl)pyridine;hexafluorophosphate (4.21 mg, 3.75 μmol, 0.015 eq), NiBr₂-dtbpy (9.13 mg, 18.75 μmol, 0.075 eq), quinuclidine (48.64 mg, 437.50 μmol, 1.75 eq), intermediate 1-2 and phthalimide (8.28 mg, 56.25 μmol, 0.225 eq). DMA (2 mL) was added to this vial under an atmosphere of nitrogen. The methyl tert-butyl ether suspension was transferred to a 5 mL syringe under air. Then a syringe filter and new needle were installed on the syringe, before the methyl tert-butyl ether solution was injected through the syringe filter into the dimethylacetamide solution. The reaction mixture was sparged with nitrogen for 15 minutes before sealing with parafilm. The vial was stirred at 1500 rpm stir rate and irradiated under 450 nm LED modules at 100% light intensity with max fan speed and 1500 rpm stirring rate in a PennOC Integrated Photoreactor for 16 hours. The reaction mixture was extracted with EtOAc (3 mL*3). The combined organic layers were washed with brine 10 mL (5 mL*2), dried over Na₂SO₄, filtered and concentrated under reduced pressure to give a residue. The residue was purified by flash silica gel chromatography (ISCO; 20 g SepaFlash Silica Flash Column, Eluent of 0˜30% Ethyl acetate/Petroleum ether gradient @40 mL/min). Intermediate 1-3 (60 mg, 165.10 μmol, 66.04% yield) was obtained as a white solid. LCMS (Method E): Rt=0.608 min, [M+H-56]⁺=308.2. ¹H NMR (400 MHz, DMSO-d6) δ=7.86-7.79 (m, 1H), 7.29-7.21 (m, 2H), 3.93-3.76 (m, 5H), 3.12-3.02 (m, 1H), 3.00 (s, 1H), 2.98-2.91 (m, 1H), 2.57 (br s, 2H), 2.54 (s, 2H), 1.50 (br d, J=5.6 Hz, 2H), 1.41 (s, 9H).

### Step 2: Synthesis of Intermediate 1-4

A solution of intermediate 1-3 (60.00 mg, 165.10 μmol, 1 eq) in HCl/dioxane (1 mL) was stirred at 25° C. for 1 hr. The reaction mixture was concentrated under reduced pressure to give crude product. The crude product was used in the next step without further purification. Intermediate 1-4 (50 mg, crude) was obtained as a white solid. LCMS (Method E): Rt=0.390 min, [M+H]⁺=264.0.

### Step 3: Synthesis of Intermediate 1-6

To a solution of intermediate 1-4 (40.00 mg, 151.91 μmol, 1 eq) and intermediate 1-5 (15.88 mg, 151.91 μmol, 13.78 μL, 1 eq) in DCM (0.5 mL) was added TEA (46.12 mg, 455.74 μmol, 63.43 μL, 3 eq). The mixture was stirred at 25° C. for 1 hr. The reaction mixture was concentrated under reduced pressure to remove solvent. The residue was extracted with EtOAc (1 mL*3). The combined organic layers were dried over anhydrous Na₂SO₄, filtered and concentrated under reduced pressure to give a residue. The residue was purified by flash silica gel chromatography (ISCO; 4 g SepaFlash Silica Flash Column, Eluent of 0˜30% Ethyl acetate/Petroleum ether gradient @12 mL/min). Intermediate 1-6 (40 mg, 120.71 μmol, 79.46% yield) was obtained as a white solid. LCMS (Method E): Rt=0.513 min, [M+H]⁺=332.2.

### Step 4: Synthesis of Intermediate 1-7

To a solution of intermediate 1-6 (30.00 mg, 90.53 μmol, 1 eq) in THF (0.5 mL) was added LiOH·H₂O (7.60 mg, 181.06 μmol, 2 eq) in H₂O (0.5 mL) and MeOH (0.5 mL). The mixture was stirred at 25° C. for 1 hr. The reaction was acidified with 0.5M HCl to pH<7, and extracted with EtOAc (2 mL*3). The combined organic layers were dried over anhydrous Na₂SO₄, filtered and concentrated under reduced pressure to give a residue. The residue was used in the next step without further purification. Intermediate 1-7 (30 mg, crude) was obtained as a white solid. LCMS (Method E): Rt=0.450 min, [M+H]⁺=318.2. ¹H NMR (400 MHz, DMSO-d6) δ=7.82-7.77 (m, 1H), 7.25-7.18 (m, 2H), 4.31-4.12 (m, 2H), 2.82 (d, J=12.8 Hz, 1H), 2.70-2.59 (m, 3H), 1.99-1.94 (m, 1H), 1.59-1.33 (m, 4H), 1.32-1.23 (m, 1H), 0.77-0.68 (m, 4H).

### Step 4: Synthesis of I-778

To a solution of intermediate 1-7 (30.00 mg, 94.53 μmol, 1 eq) and intermediate 1-8 (72.44 mg, 94.53 μmol, 1 eq, HCl salt) in DMF (1 mL) was added EDCI (36.24 mg, 189.06 μmol, 2 eq) NMM (47.81 mg, 472.66 μmol, 51.97 μL, 5 eq) and HOAt (12.87 mg, 94.53 μmol, 13.22 μL, 1 eq). The mixture was stirred at 25° C. for 1 hr. The reaction mixture was diluted with MeOH (0.5 mL) and purified directly. The residue was purified by prep-HPLC (column: Phenomenex luna C18 150*25 mm*10 um; mobile phase: [water (FA)-ACN]; gradient: 23%-53% B over 10 min). I-778 (54 mg, 52.47 μmol, 55.50% yield) was obtained as a white solid. LCMS (Method E): Rt=0.504 min, [M+H]⁺=1029.8. SFC: Rt=5.674 min. ¹H NMR (400 MHz, DMSO-d6) δ=12.60 (s, 1H), 8.30-8.16 (m, 2H), 8.00-7.94 (m, 1H), 7.92-7.79 (m, 2H), 7.61-7.52 (m, 1H), 7.48-7.42 (m, 1H), 7.40-7.35 (m, 1H), 7.30-7.12 (m, 3H), 4.87-4.71 (m, 1H), 4.38-4.10 (m, 4H), 4.01-3.80 (m, 2H), 3.74-3.36 (m, 10H), 3.30-2.98 (m, 6H), 2.88-2.74 (m, 2H), 2.67-2.59 (m, 2H), 2.33-1.94 (m, 2H), 1.71 (d, J=9.6 Hz, 8H), 1.63-1.21 (m, 10H), 1.20-0.95 (m, 5H), 0.85-0.59 (m, 4H). ¹⁹F NMR (377 MHz, DMSO-d6) δ=−113.91 (br s, 1F), −119.75 (br s, 1F).

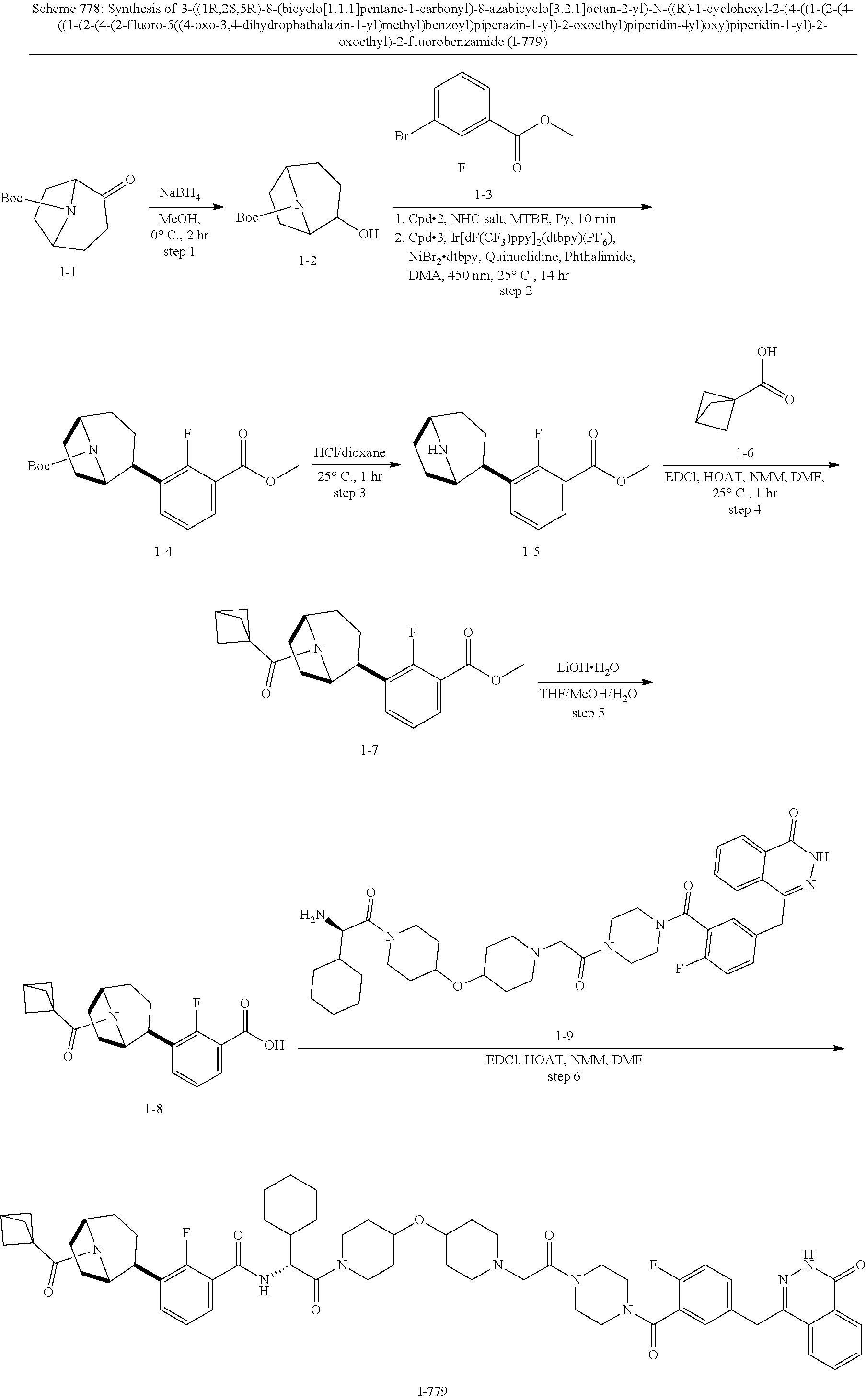

### Step 1: Synthesis of Intermediate 1-2

To a solution of Intermediate 1-1 (1 g, 4.44 mmol, 1 eq) in MeOH (10 mL) was added NaBH₄ (335.86 mg, 8.88 mmol, 2 eq) at 0° C. under N₂ atmosphere. The mixture was stirred at 0° C. for 2 hr. The reaction mixture was quenched with NH₄Cl saturated aqueous solution (15 mL) and extracted with ethyl acetate (10 mL*3). The organic phases were combined, washed with saturated brine (10 mL), dried over anhydrous sodium sulfate and concentrated to obtain compound. The crude product was used in the next step without further purification. Intermediate 1-2 (1 g, 4.40 mmol, 99.11% yield) was obtained as a colorless oil. LCMS (Method D): Rt=0.286 min, [M−55]⁺=1024.6. ¹H NMR (400 MHz, CHLOROFORM-d) δ=4.35-4.16 (m, 1H), 4.10 (s, 1H), 3.94-3.69 (m, 1H), 2.02-1.91 (m, 2H), 1.90 (s, 3H), 1.76-1.68 (m, 1H), 1.66-1.51 (m, 2H), 1.47 (s, 9H).

### Step 2: Synthesis of Intermediate 1-4

To a 40 mL vial equipped with a stir bar was added Intermediate 1-2 (1 g, 4.40 mmol, 1.75 eq) and NHC salt (1.59 g, 4.02 mmol, 1.6 eq) in MTBE (9 mL). Then pyridine (318.17 mg, 4.02 mmol, 324.66 L, 1.6 eq) in MTBE (1 mL) was added drop by drop. The vial was sealed and placed in a glovebox and stirred at 25° C. for 10 mins, and then filtered. Ir(ppy)₂(dtbpy)(PF₆) (34.47 mg, 37.71 μmol, 0.015 eq), NiBr₂-dtbpy (91.80 mg, 188.55 μmol, 0.075 eq), quinuclidine (489.15 mg, 4.40 mmol, 1.75 eq) and Intermediate 1-3 (585.85 mg, 2.51 mmol, 1 eq) in DMA (13 mL) were then added to the above filtered mixture. The reaction was stirred and irradiated with a 10 W 455 nm blue LED lamp (3 cm away), with cooling water to keep the reaction temperature at 25° C. for 14 hr. The reaction mixture was diluted with water (10 mL) and extracted with DCM (10 mL*3). The combined organic layers were dried over Na₂SO₄, filtered and concentrated under reduced pressure to give a residue. The residue was purified by flash silica gel chromatography (ISCO; 40 g SepaFlash Silica Flash Column, Eluent of 0-100% Petroleum ether/Ethyl acetate @40 mL/min). The eluent was concentrated under reduced pressure to give a residue. Intermediate 1-4 (620 mg, 1.68 mmol, 67.01% yield, 98.745% purity) was obtained as a white solid. LCMS: Rt=0.479 min, [M+Na]=386.1. SFC: Rt=0.557&0.681 min.

### Step 3: Synthesis of Intermediate 1-5

A solution of Intermediate 1-4 (200 mg, 550.32 μmol, 1 eq) in HCl/dioxane (2 M, 2.00 mL, 7.27 eq) was stirred at 25° C. for 0.5 hr. The residue was concentrated under reduced pressure to give a residue. The residue was used in the next step without further purification. Intermediate 1-5 (260 mg, crude, HCl salt) was obtained as a yellow oil. LCMS (Method D): Rt=0.225 min, [M+H]⁺=264.0.

### Step 4: Synthesis of Intermediate 1-7

To a solution of Intermediate 1-6 (74.81 mg, 667.18 μmol, 1 eq) in DMF (2 mL) was added HOAt (90.81 mg, 667.18 μmol, 93.33 μL, 1 eq), EDCI (383.70 mg, 2.00 mmol, 3 eq), NMM (337.42 mg, 3.34 mmol, 366.76 μL, 5 eq), and Intermediate 1-5. The mixture was stirred at 25° C. for 1 hr. The reaction mixture was diluted with water (10 mL) and extracted with DCM (10 mL*3). The combined organic layers were dried over Na₂SO₄, filtered and concentrated under reduced pressure to give a residue. The residue was purified by flash silica gel chromatography (ISCO; 20 g SepaFlash Silica Flash Column, Eluent of 0˜100% Petroleum ether/Ethyl acetate @40 mL/min) and the eluent was concentrated under reduced pressure. Intermediate 1-7 (310 mg, crude) was obtained as a yellow oil. LCMS (Method D): Rt=0.433 min, [M+H]⁺=358.0. SFC: Rt=1.223&1.812 min. ¹H NMR (400 MHz, METHANOL-d4) δ=7.98 (s, 1H), 7.86-7.76 (m, 1H), 7.74-7.57 (m, 1H), 7.32-7.24 (m, 1H), 4.70-4.57 (m, 2H), 3.91 (d, J=9.6 Hz, 3H), 3.39 (br s, 1H), 2.99 (s, 3H), 2.86 (d, J=0.8 Hz, 3H), 2.49 (d, J=4.0 Hz, 1H), 2.27-2.16 (m, 7H).

### Step 5: Synthesis of Intermediate 1-8

To a solution of Intermediate 1-7 (170 mg, 475.63 μmol, 1 eq) in THF (1.7 mL), MeOH (0.85 mL) and H₂O (1.7 mL) was added LiOH·H₂O (59.88 mg, 1.43 mmol, 3 eq). The mixture was stirred at 25° C. for 1 hr. The reaction mixture was diluted with water (5 mL) and concentrated to remove THF and MeOH, and then adjusted to pH to 3-4 with aq·HCl (1 M) and extracted with EA (10 mL*3). The combined organic layers were dried over anhydrous Na₂SO₄, and concentrated under reduced pressure. Intermediate 1-8 (140 mg, 402.37 μmol, 84.60% yield, 98.694% purity) was obtained as a light yellow solid. LCMS (Method E): Rt=0.480 min, [M+H]⁺=344.1. ¹H NMR (400 MHz, DMSO-d6) δ=7.78-7.55 (m, 2H), 7.32-7.20 (m, 1H), 4.56-4.49 (m, 1H), 4.48-4.39 (m, 1H), 3.26-3.19 (m, 1H), 2.22-2.05 (m, 7H), 2.03-1.88 (m, 1H), 1.88-1.39 (m, 7H).

### Step 6: Synthesis of I-779

To a solution of Intermediate 1-8 (32.26 mg, 93.95 μmol, 0.9 eq), HOAt (14.21 mg, 104.39 μmol, 14.60 μL, 1 eq), EDCI (40.02 mg, 208.78 μmol, 2 eq), and NMM (52.80 mg, 521.96 μmol, 57.39 μL, 5 eq) in DMF (0.8 mL) was added Intermediate 1-9 (80 mg, 104.39 μmol, 1 eq, HCl salt). The mixture was stirred at 25° C. for 1 hr. The reaction mixture was diluted with water (20 mL) and extracted with EA (10 mL*3). The combined organic layer was washed with brine (10 mL), dried over Na₂SO₄, filtered and concentrated to give a residue. The residue was purified by Prep-HPLC (column: Waters Xbridge 150*25 mm*5 um; mobile phase: water (NH₄HCO₃)-ACN; B: acetonitrile; gradient: 35% to 65% B in 15 min, flow rate: 25 mL/min), and then lyophilized. I-779 (44.47 mg, 42.14 μmol, 40.37% yield, 100.00% purity) was obtained as a off-white solid. LCMS (Method G): Rt=1.708 min, [M+H]⁺=1055.6. SFC: Rt=3.726 min, 5.317 min. ¹H NMR (400 MHz, DMSO-d6) δ=12.60 (s, 1H), 8.63-8.30 (m, 1H), 8.26 (d, J=7.6 Hz, 1H), 8.00-7.93 (m, 1H), 7.92-7.86 (m, 1H), 7.86-7.80 (m, 1H), 7.61-7.47 (m, 1H), 7.47-7.33 (m, 3H), 7.30-7.17 (m, 2H), 4.85-4.72 (m, 1H), 4.60-4.40 (m, 2H), 4.33 (s, 2H), 4.04-3.76 (m, 2H), 3.72-3.53 (m, 4H), 3.53-3.44 (m, 2H), 3.41-3.35 (m, 2H), 3.25-3.03 (m, 6H), 2.66-2.59 (m, 1H), 2.46-2.38 (m, 1H), 2.19-1.92 (m, 10H), 1.86-1.54 (m, 17H), 1.49-0.93 (m, 10H). ¹⁹F NMR (377 MHz, DMSO-d6) δ=−116.41-−118.68 (m, 1F), −119.77 (s, 1F).

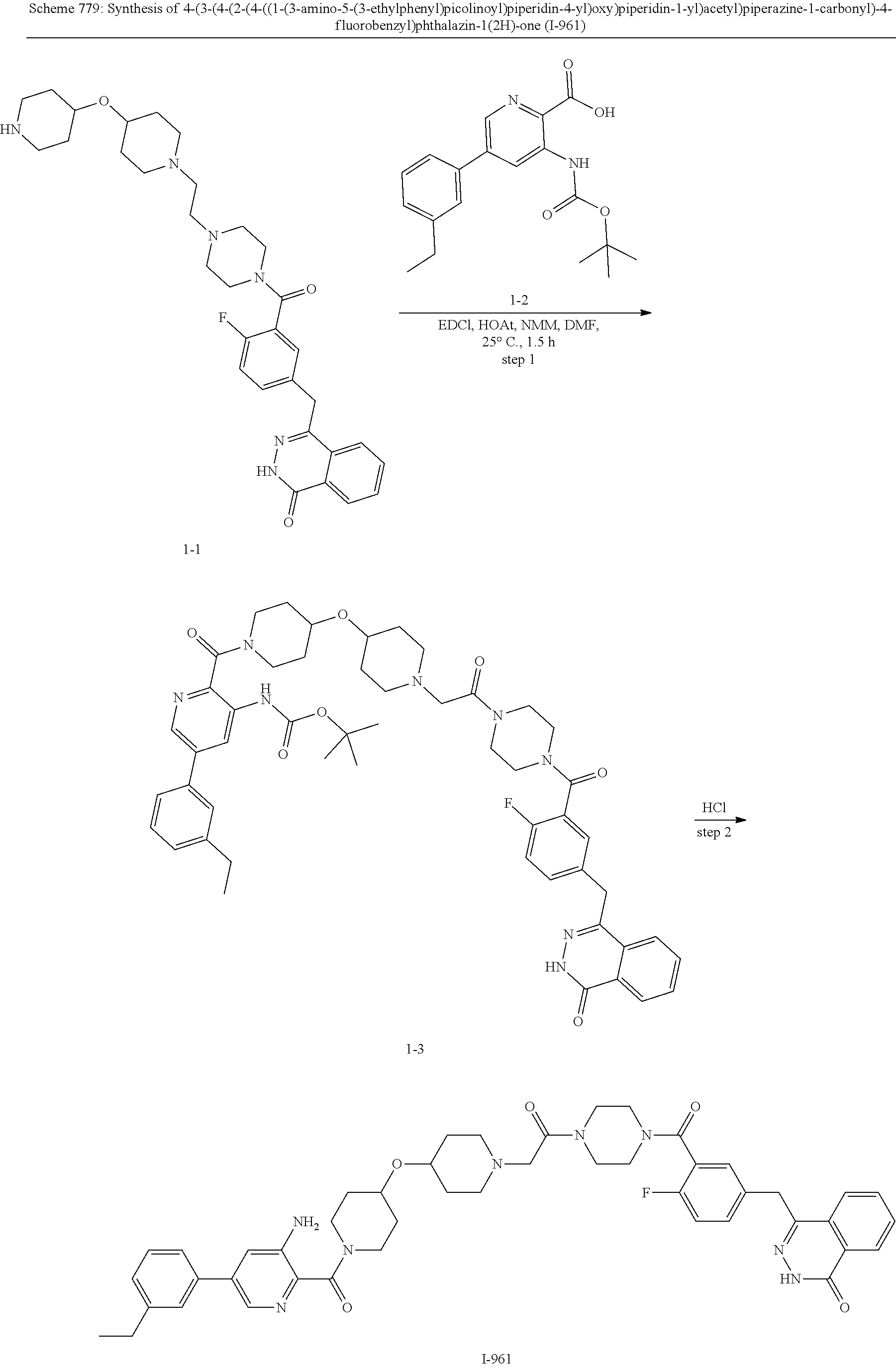 [SMILES: O=C(c1cc(Cc2n[nH]c(=O)c3ccccc23)ccc1F)N1CCN(CCN2CCC(OC3CCNCC3)CC2)CC1 ; CCc1cccc(-c2cnc(C(=O)O)c(NC(=O)OC(C)(C)C)c2)c1 ; CCc1cccc(-c2cnc(C(=O)N3CCC(OC4CCN(CC(=O)N5CCN(C(=O)c6cc(Cc7n[nH]c(=O)c8ccccc78)ccc6F)CC5)CC4)CC3)c(NC(=O)OC(C)(C)C)c2)c1 ; Cl ; CCc1cccc(-c2cnc(C(=O)N3CCC(OC4CCN(CC(=O)N5CCN(C(=O)c6cc(Cc7n[nH]c(=O)c8ccccc78)ccc6F)CC5)CC4)CC3)c(N)c2)c1]

### Step 1: Synthesis of Intermediate 1-1

To a solution of Intermediate 1-1 (150 mg, 253.94 μmol, 1 eq) and Intermediate 1-2 (86.95 mg, 253.94 μmol, 1 eq) in DMF (1.5 mL) was added EDCI (97.36 mg, 507.88 μmol, 2 eq), HOAt (34.56 mg, 253.94 μmol, 35.52 μL, 1 eq) and NMM (128.43 mg, 1.27 mmol, 139.59 μL, 5 eq). The mixture was stirred at 25° C. for 1 hr. To the reaction mixture was added to H₂O (10 ml), and solid precipitated. The reaction mixture was filtered and the cake was dried under reduced pressure to give a residue. The crude was used in the next step. Intermediate 1-3 (120 mg, 131.14 μmol, 51.64% yield) was obtained as a white solid. LCMS (Method D): Retention time: 0.412 min, (M+H)=915.5.

### Step 2: Synthesis of I-961

A solution of Intermediate 1-4 (40 mg, 43.71 μmol, 1 eq) in DCM (0.2 mL) was added HCl/dioxane (2 M, 0.2 mL, 9.15 eq) was stirred at 25° C. for 1 hr. The reaction mixture was concentrated under reduced pressure to give a residue. The crude product was purified by reversed-phase HPLC (0.1% FA condition), and the eluent was concentrated to remove ACN and lyophilized to give product. I-961 (12.86 mg, 14.61 μmol, 33.42% yield, 97.791% purity, FA salt) was obtained as a white solid. LCMS (Method D): Retention time: 0.349 min, (M+H)=815.4. ¹H NMR (400 MHz, METHANOL-d₄) δ=8.43 (s, 1H), 8.39-8.34 (m, 1H), 8.08 (d, J=1.6 Hz, 1H), 7.98-7.91 (m, 1H), 7.91-7.79 (m, 2H), 7.54-7.47 (m, 1H), 7.47-7.33 (m, 5H), 7.27 (d, J=7.2 Hz, 1H), 7.17 (t, J=9.2 Hz, 1H), 4.39 (s, 2H), 4.14-3.99 (m, 1H), 3.88-3.40 (m, 13H), 3.35 (s, 2H), 3.16-2.96 (m, 2H), 2.82-2.61 (m, 4H), 2.04-1.58 (m, 8H), 1.28 (t, J=7.6 Hz, 3H). ¹⁹F NMR (400 MHz, METHANOL-d₄) δ=−120.8.

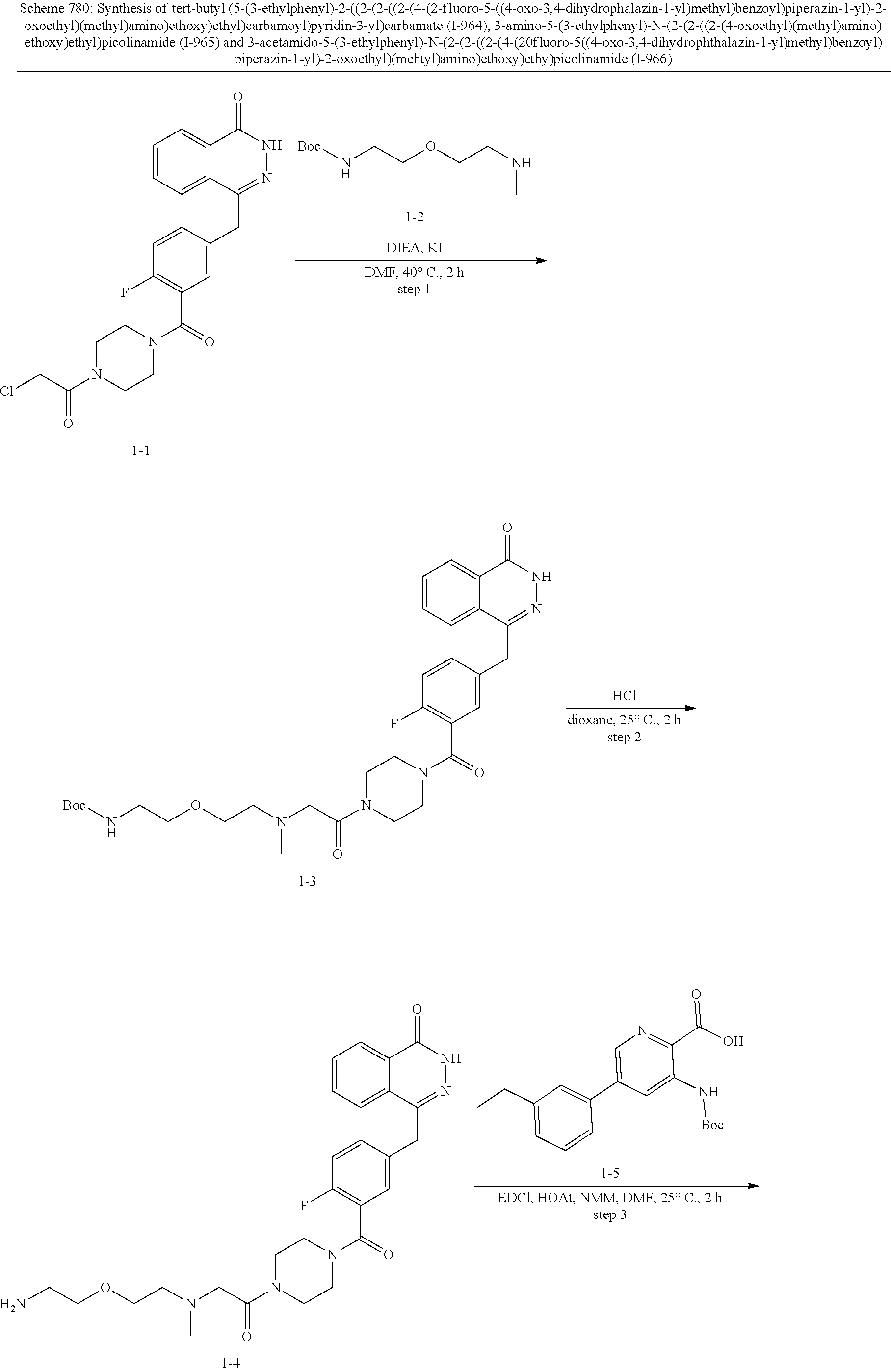

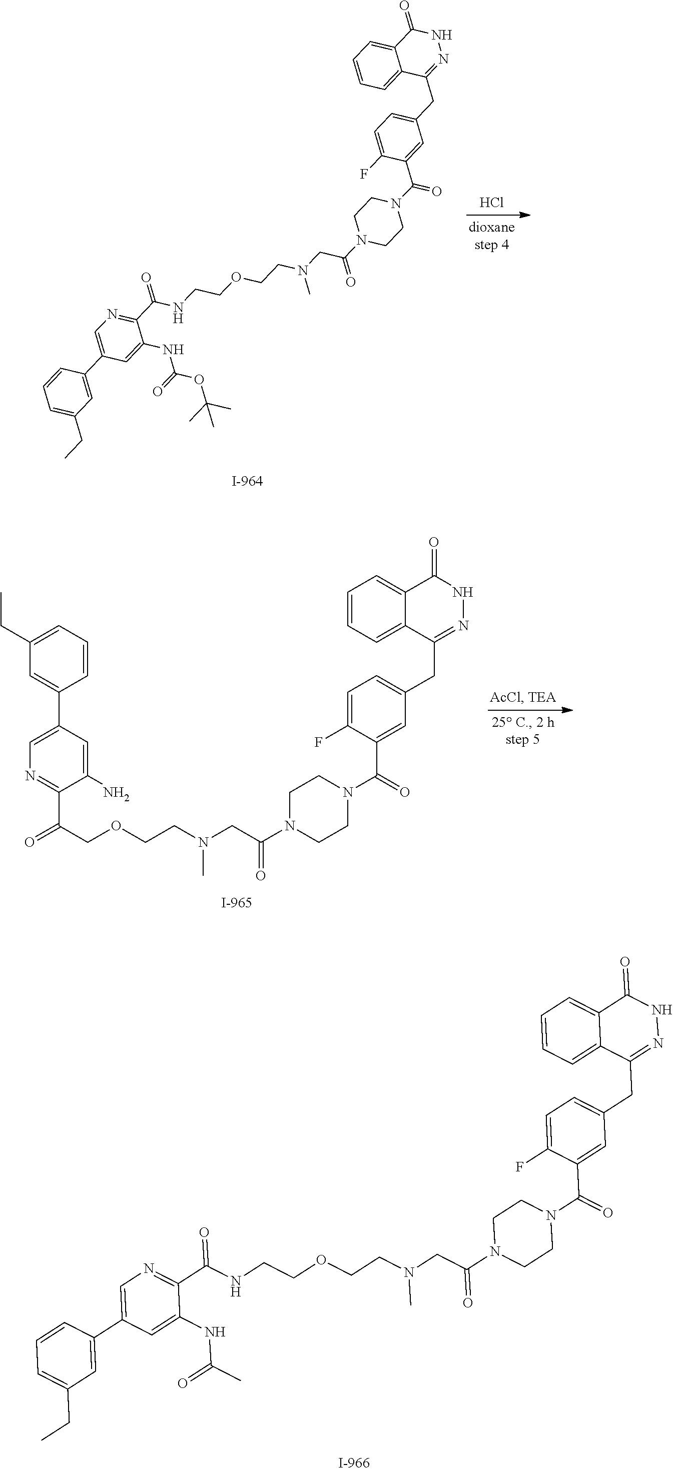 [SMILES: CCc1cccc(-c2cnc(C(=O)NCCOCCN(C)CC(=O)N3CCN(C(=O)c4cc(Cc5n[nH]c(=O)c6ccccc56)ccc4F)CC3)c(NC(=O)OC(C)(C)C)c2)c1 ; Cl ; CCc1cccc(-c2cnc(C(=O)COCCN(C)CC(=O)N3CCN(C(=O)c4cc(Cc5n[nH]c(=O)c6ccccc56)ccc4F)CC3)c(N)c2)c1 ; CCc1cccc(-c2cnc(C(=O)NCCOCCN(C)CC(=O)N3CCN(C(=O)c4cc(Cc5n[nH]c(=O)c6ccccc56)ccc4F)CC3)c(NC(C)=O)c2)c1]

### Step 1: Synthesis of Intermediate 1-3

To a solution of Intermediate 1-1 (500 mg, 1.13 mmol, 1 eq) in DMF (5 mL) was added Intermediate 1-2 (369.68 mg, 1.69 mmol, 1.5 eq), DIEA (583.66 mg, 4.52 mmol, 786.60 μL, 4 eq) and KI (749.67 mg, 4.52 mmol, 4 eq). The mixture was stirred at 40° C. for 2 hr. The reaction mixture was diluted with water (40 mL) and extracted with EA (15 mL*3). The combined organic layers were washed with a sat. NaCl solution (15 mL), dried over Na₂SO₄, filtered and concentrated under reduced pressure to give a residue. The residue was purified by reversed phase chromatography (26.8*125 mm, 80 g of XB—C18, 20-40 μm, 120 Å) Mobile phase: A for H₂O (0.1% FA v/v) and B for acetonitrile; Gradient: B 0%-100% in 15 min; Flow rate: 40 mL/min; Column temperature: R.T. Wavelength: 220 nm/254 nm). The eluent was concentrated under reduced pressure to give a product. Intermediate 1-3 (0.7 g, 1.11 mmol, 98.26% yield, 99% purity) was obtained as a brown oil. LCMS (Method E): Rt=0.426 min, (M+H=625.3). ¹H NMR (400 MHz, CHLOROFORM-d) δ=10.90-10.50 (m, 1H), 8.50-8.43 (m, 1H), 7.86-7.69 (m, 3H), 7.33 (d, J=6.0 Hz, 2H), 7.11-7.01 (m, 1H), 5.54-5.31 (m, 1H), 4.87-4.74 (m, 4H), 4.30 (s, 2H), 3.87-3.76 (m, 2H), 3.71 (s, 2H), 3.59-3.48 (m, 4H), 3.40-3.22 (m, 4H), 3.12-2.94 (m, 2H), 2.61 (d, J=19.6 Hz, 3H), 1.43 (d, J=9.2 Hz, 9H).

### Step 2: Synthesis of Intermediate 1-4

To a solution of Intermediate 1-3 (600 mg, 960.46 μmol, 1 eq) in dioxane (6 mL) was added HCl/dioxane (2 M, 6.00 mL, 12.49 eq). The mixture was stirred at 25° C. for 2 hr. The reaction mixture was concentrated under reduced pressure to remove dioxane. Intermediate 1-4 (600 mg, crude) was obtained as a yellow oil. LCMS (Method E): Rt=0.349 min, (M+H=525.3).

### Step 3: Synthesis of I-964

To a solution of Intermediate 1-4 (600 mg, 1.14 mmol, 1 eq) in DMF (6 mL) was added Intermediate 1-5 (469.93 mg, 1.37 mmol, 1.2 eq), HOAt (311.36 mg, 2.29 mmol, 320.00 μL, 2 eq), NMM (347.06 mg, 3.43 mmol, 377.24 μL, 3 eq) and EDCI (438.52 mg, 2.29 mmol, 2 eq). The mixture was stirred at 25° C. for 2 hr. The reaction mixture was diluted with water, then filtered and the filtered cake was dried under reduced pressure to give a residue. The residue was purified by reversed phase chromatography (26.8*125 mm, 80 g of XB—C18, 20-40 μm, 120 Å) Mobile phase: A for H₂O (0.1% FA v/v) and B for acetonitrile; Gradient: B 0%-100% in 15 min; Flow rate: 40 mL/min; Column temperature: R.T. Wavelength: 220 nm/254 nm). The eluent was concentrated under reduced pressure to give a product. I-964 (320 mg, 376.93 μmol, 32.96% yield, 100% purity) was obtained as a white solid. LCMS (Method E): Rt=0.567 min, (M+23=871.4). ¹H NMR (400 MHz, METHANOL-d4) δ=9.04-8.94 (m, 1H), 8.49-8.30 (m, 2H), 7.92-7.75 (m, 3H), 7.54-7.46 (m, 2H), 7.45-7.35 (m, 2H), 7.34-7.25 (m, 2H), 7.14-7.02 (m, 1H), 4.30 (s, 2H), 3.75-3.57 (m, 10H), 3.54-3.47 (m, 2H), 3.46-3.39 (m, 2H), 3.27-3.09 (m, 2H), 2.80-2.64 (m, 4H), 2.42-2.28 (m, 3H), 1.54 (d, J=4.0 Hz, 9H), 1.32-1.22 (m, 3H).

### Step 4: Synthesis of I-965

To a solution of I-964 (30 mg, 35.34 μmol, 1 eq) was added HCl/dioxane (4 M, 0.5 mL, 56.60 eq). The mixture was stirred at 25° C. for 2 hr. The reaction mixture was concentrated to remove dioxane to give a residue. The residue was purified by reversed phase chromatography (26.8*125 mm, 40 g of XB—C18, 20-40 μm, 120 Å) Mobile phase: A for H₂O (0.1% FA v/v) and B for acetonitrile; Gradient: B 0%-100% in 15 min; Flow rate: 40 mL/min; Column temperature: R.T. Wavelength: 220 nm/254 nm). The eluent was concentrated under vacuum to give a product. I-965 (14.5 mg, 19.36 μmol, 54.80% yield, 100% purity) was obtained as a white solid. LCMS (Method E): Rt=0.497 min, (M+23=771.4). ¹H NMR (400 MHz, METHANOL-d4) δ=8.39-8.30 (m, 1H), 8.10-7.94 (m, 1H), 7.92-7.75 (m, 3H), 7.46-7.36 (m, 4H), 7.36-7.19 (m, 4H), 7.13-7.02 (m, 1H), 4.31 (s, 2H), 3.75 (d, J=2.0 Hz, 1H), 3.71-3.63 (m, 5H), 3.62-3.53 (m, 5H), 3.52-3.39 (m, 4H), 3.26-3.15 (m, 1H), 2.77-2.63 (m, 4H), 2.42-2.32 (m, 3H), 1.31-1.20 (m, 3H).

### Step 5: Synthesis of I-966

To a solution of I-965 (50 mg, 66.77 μmol, 1 eq) in DCM (0.5 mL) was added acetyl chloride (10.48 mg, 133.54 μmol, 9.50 μL, 2 eq) and TEA (20.27 mg, 200.31 μmol, 27.88 μL, 3 eq). The mixture was stirred at 25° C. for 2 hr. The reaction mixture was diluted with water (10 mL) and extracted with EA (10 mL*3). The combined organic layers were dried over Na₂SO₄, filtered and concentrated under reduced pressure to give a residue. The residue was purified by prep-HPLC (column: Phenomenex luna C18 150*25 mm*10 um; mobile phase: [water(FA)-ACN]; gradient: 18%-48% B over 15 min). The eluent was concentrated and lyophilized to afford the desired product. I-966 (18.08 mg, 22.86 μmol, 34.24% yield, 100% purity) was obtained as a white solid. LCMS (Method E): Rt=0.495 min, (M+H=791.4). ¹H NMR (400 MHz, METHANOL-d4) δ=9.23 (d, J=9.2 Hz, 1H), 8.57-8.42 (m, 1H), 8.38-8.30 (m, 1H), 7.91-7.76 (m, 3H), 7.52-7.36 (m, 4H), 7.34-7.25 (m, 2H), 7.13-7.02 (m, 1H), 4.30 (s, 2H), 3.79-3.58 (m, 10H), 3.56-3.48 (m, 3H), 3.41 (s, 2H), 3.24-3.16 (m, 1H), 2.81-2.65 (m, 4H), 2.49-2.37 (m, 3H), 2.37-2.21 (m, 3H), 1.32-1.21 (m, 3H).

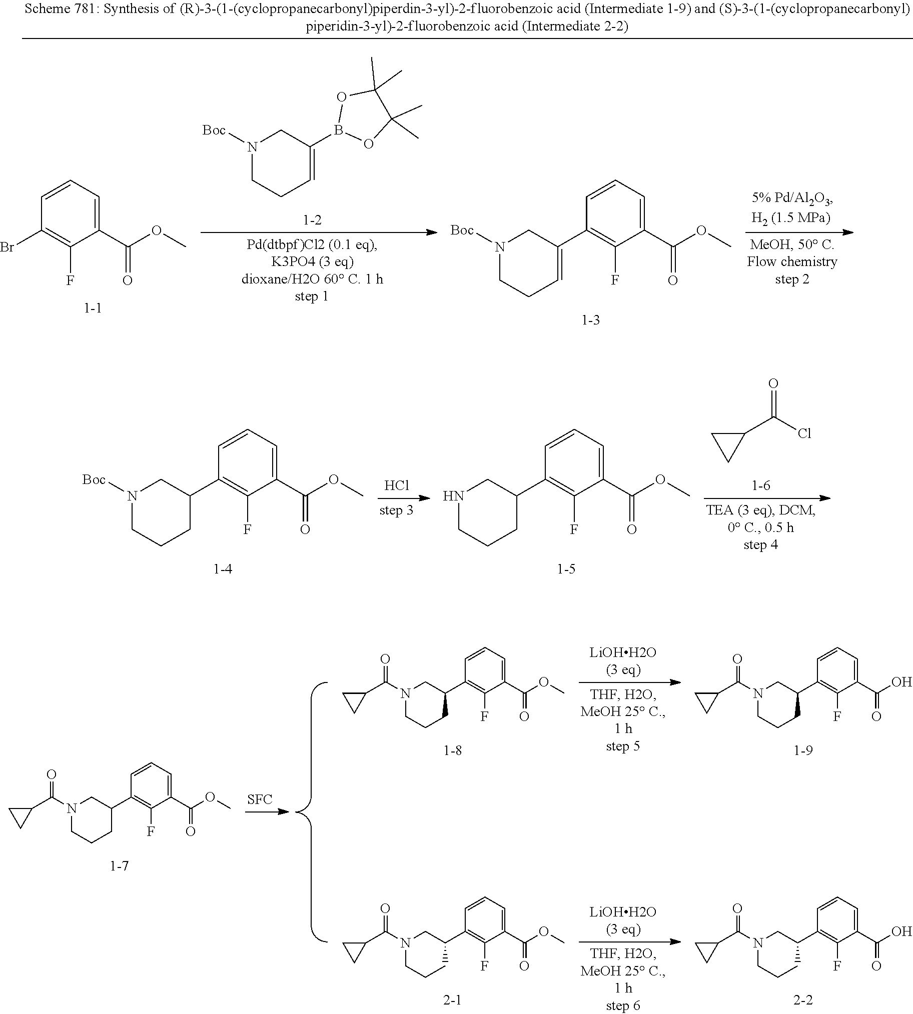

### Step 1: Synthesis of Intermediate 1-3

A mixture of intermediate 1-1 (50 g, 214.56 mmol, 1 eq), intermediate 1-2 (79.61 g, 257.47 mmol, 1.2 eq), K₃PO₄ (136.63 g, 643.68 mmol, 3 eq), ditert-butyl(cyclopenta-1,4-dien-1-yl)phosphane;dichloropalladium;iron (6.99 g, 10.73 mmol, 0.05 eq) in dioxane (500 mL) and H₂O (50 mL) was degassed and purged with N₂ 3 times, and then the mixture was stirred at 50° C. for 12 h under N₂ atmosphere. The reaction was diluted with H₂O (500 mL), extracted with ethyl acetate 1.5 L (500 mL*3), and then dried over Na₂SO₄, filtered and concentrated under reduced pressure to give a residue. The residue was purified by flash silica gel chromatography (ISCO; 550 g SepaFlash Silica Flash Column, Eluent of 0˜40% Ethyl acetate/Petroleum ether gradient @200 mL/min). The eluent was concentrated to afford Intermediate 1-1 (80 g, crude) as a yellow oil. LCMS (Method E): Rt=0.593 min, [M+Na]⁺=358.2. ¹H NMR (400 MHz, CHLOROFORM-d) δ=7.83 (br t, J=6.8 Hz, 1H), 7.47-7.39 (m, 1H), 7.17 (t, J=7.6 Hz, 1H), 6.03 (s, 1H), 4.26-4.14 (m, 2H), 3.94 (s, 3H), 3.58 (t, J=6.0 Hz, 2H), 2.33 (br d, J=2.8 Hz, 2H), 1.49 (s, 9H)

### Step 2: Synthesis of Intermediate 1-4

Intermediate 1-1 (1 eq, 70 g) was dissolved in MeOH (1750 mL). A fixed bed (volume 50 mL) was packed with granular catalyst (5% Pd/Al₂O₃, 20 g). The H₂ back pressure regulator was adjusted to 1.5 MPa and the flow rate of H₂ was 90 mL/min. Then the solution of Intermediate 1-1 was pumped to the fixed bed (10 mL, 50° C.). The solution was flowing through (4 mL/min) the reactor zone, and then the reaction mixture was collected from the reactor output. The mixture was concentrated under vacuum to get a residue. Intermediate 1-4 (70 g, 207.48 mmol, 100.00% yield) was a white solid, and it was used in the next step without further purification. LCMS (Method E): Rt=0.572 min, [M+Na]⁺=360.1. ¹H NMR (400 MHz, CHLOROFORM-d) δ=7.85-7.75 (m, 1H), 7.46-7.36 (m, 1H), 7.16 (t, J=8.0 Hz, 1H), 4.29-4.07 (m, 2H), 3.94 (s, 3H), 3.11-2.99 (m, 1H), 2.83-2.72 (m, 1H), 2.07-1.93 (m, 1H), 1.85-1.55 (m, 4H), 1.50-1.46 (m, 9H).

### Step 3: Synthesis of Intermediate 1-5

To a solution of intermediate 1-4 (9 g, 26.68 mmol, 1 eq) in DCM (90 mL) was added HCl/dioxane (2 M, 90 mL). The mixture was stirred at 25° C. for 1 h. The reaction mixture was concentrated under reduced pressure to give a residue and used in the next step without further purification. Intermediate 1-5 (7.2 g, 24.20 mmol, 90.72% yield, 92% purity, HCl salt) was obtained as a white solid. LCMS (Method E): Rt=0.365 min, [M+H]⁺=238.0.

Step 4: Synthesis of Intermediate 1-7, intermediate 1-8 and intermediate 2-1

To a solution of intermediate 1-5 (8 g, 29.23 mmol, 1 eq, HCl salt) and intermediate 1-6 (6.11 g, 58.45 mmol, 5.30 mL, 2 eq) in DCM (80 mL) was added TEA (8.87 g, 87.68 mmol, 12.20 mL, 3 eq). The mixture was stirred at 0° C. for 0.5 h. The reaction mixture was quenched by addition of H₂O (50 mL), and then diluted with DCM (50 mL) and extracted with DCM (100 mL*3). The combined organic layers were dried over anhydrous Na₂SO₄, filtered and concentrated under reduced pressure to give a residue. The residue was purified by flash silica gel chromatography (ISCO; 120 g SepaFlash Silica Flash Column, Eluent of 0˜100% Ethyl acetate/Petroleum ether gradient @100 mL/min) and then the eluent was concentrated under reduced pressure to give intermediate 1-7 (4 g, 12.71 mmol, 43.48% yield, 97% purity). LCMS (Method E): Rt=505 min, [M+H]⁺=306.0. ¹H NMR (400 MHz, DMSO-d6) δ=7.81-7.51 (m, 2H), 7.30 (s, 1H), 4.51-4.22 (m, 2H), 3.85 (s, 3H), 3.28-3.08 (m, 1H), 3.07-2.84 (m, 1H), 2.74-2.57 (m, 1H), 2.08-1.69 (m, 4H), 1.64-1.36 (m, 1H), 0.71 (d, J=6.0 Hz, 4H). SFC: Rt=1.241 min, Rt=1.470 min/SFC separation: 3 g of intermediate 1-7 was purified by SFC separation (column: (s,s) WHELK-01 (250 mm*30 mm, 10 um);mobile phase: [CO₂-MeOH (0.1% NH₃H₂O)]; B %: 25%, isocratic elution mode) to afford Peak 1 (intermediate 1-8) and Peak 2 (intermediate 2-1). Intermediate 1-8 (1.5 g, 4.86 mmol, 16.64% yield, 99% purity) was obtained as a colorless oil. LCMS (Method E): Rt=0.511 min, [M+H]+=306.0. SFC: Rt=1.247 min, ee value=99%. Intermediate 2-1 (1.5 g, 4.77 mmol, 16.30% yield, 97% purity) was obtained as a colorless oil. LCMS (Method E): Rt=0.510 min, [M+H]⁺=306.0. SFC: Rt=1.472 min, ee value=99%.

### Step 5: Synthesis of Intermediate 1-9

To a solution of intermediate 1-8 (1.4 g, 4.59 mmol, 1 eq) in MeOH (6 mL), THF (6 mL) and H₂O (3 mL) was added LiOH·H₂O (577.21 mg, 13.76 mmol, 3 eq). The mixture was stirred at 25° C. for 1 h. The reaction mixture was adjusted to pH=6 with aq·HCl (1 M), and then it was diluted with EA (10 mL) and extracted with EA (10 mL*3). The combined organic layers were dried over anhydrous Na₂SO₄ and concentrated under reduced pressure to give a residue. The residue was purified by flash silica gel chromatography (ISCO; 40 g SepaFlash Silica Flash Column, Eluent of 0˜100% Ethyl acetate:Methanol@60 mL/min), then the eluent was concentrated under reduced pressure to give intermediate 1-9 (1 g, 3.40 mmol, 74.12% yield, 99% purity) as a white solid. LCMS (Method E): Rt=0.454 min, [M+H]⁺=292.0. SFC: Rt=1.766 min, ee value=99%. ¹H NMR (400 MHz, DMSO-d6) δ=7.47 (d, J=4.4 Hz, 1H), 7.37-7.24 (m, 1H), 7.07 (d, J=2.4 Hz, 1H), 4.47-4.20 (m, 2H), 3.21-3.08 (m, 1H), 2.98-2.78 (m, 1H), 2.69-2.56 (m, 1H), 2.05-1.68 (m, 4H), 1.61-1.37 (m, 1H), 0.72 (s, 4H). ¹⁹F NMR (377 MHz, DMSO-d6) δ=−120.68 (m, 1F).

### Step 6: Synthesis of Intermediate 2-2

To a solution of intermediate 2-1 (1.4 g, 4.59 mmol, 1 eq) in MeOH (6 mL), THF (6 mL) and H₂O (3 mL) was added LiOH·H₂O (577.16 mg, 13.76 mmol, 3 eq). The mixture was stirred at 25° C. for 1 h. The reaction mixture was adjusted to pH=6 with aq·HCl (1 M), and then diluted with EA (10 mL) and extracted with EA (10 mL*3). The combined organic layers were dried over anhydrous Na₂SO₄, and concentrated under reduced pressure to give a residue. The residue was purified by flash silica gel chromatography (ISCO; 40 g SepaFlash Silica Flash Column, Eluent of 0˜100% Ethyl acetate:Methanol @60 mL/min), then the eluent was concentrated under reduced pressure to give intermediate 2-2 (1.2 g, 4.12 mmol, 89.84% yield, 100% purity) as a white solid. LCMS (Method E): Rt=0.450 min, [M+H]⁺=292.0. SFC: Rt=1.911 min. ¹H NMR (400 MHz, DMSO-d6) δ=7.49 (d, J=6.8 Hz, 1H), 7.38-7.26 (m, 1H), 7.08 (d, J=6.8 Hz, 1H), 4.45-4.19 (m, 2H), 3.22-3.06 (m, 1H), 3.00-2.77 (m, 1H), 2.70-2.55 (m, 1H), 2.04-1.69 (m, 4H), 1.59-1.36 (m, 1H), 0.71 (d, J=4.4 Hz, 4H). ¹⁹F NMR (377 MHz, DMSO-d6) δ=−120.43 (m, 1F).

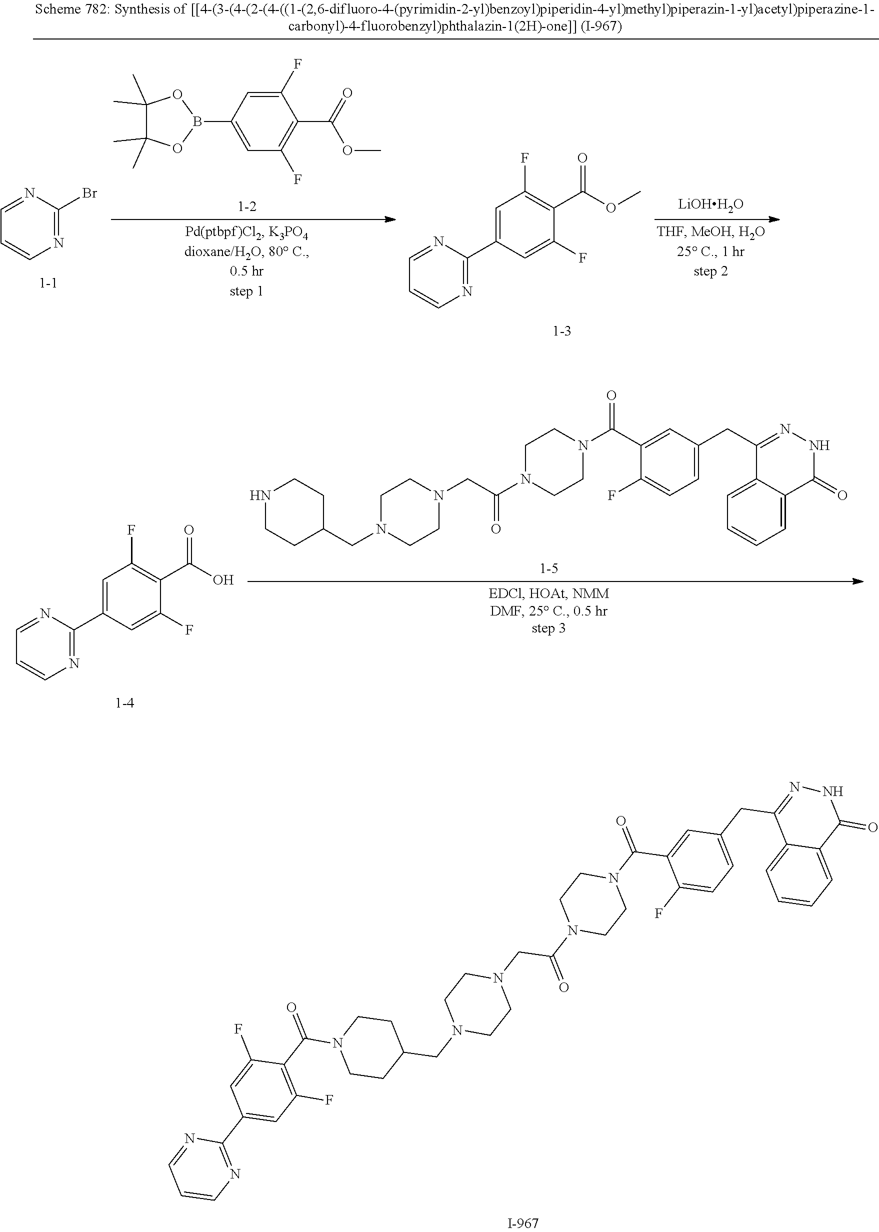

### Step 1: Synthesis of Intermediate 1-3

To the mixture of Intermediate 1-1 (500 mg, 3.14 mmol, 1 eq), Intermediate 1-2 (937.48 mg, 3.14 mmol, 1 eq) and K₃PO₄ (2.00 g, 9.43 mmol, 3 eq) in dioxane (5 mL) and H₂O (1 mL) was added Pd(dtbpf)Cl₂ (204.97 mg, 314.50 μmol, 0.1 eq) under N₂. The reaction mixture was stirred at 80° C. for 0.5 hr. The reaction mixture was diluted with water (20 mL) and extracted with EA (10 mL*3), the combined organic phase was dried over anhydrous Na₂SO₄ and concentrated to give the residue. The residue was purified by flash silica gel chromatography (ISCO; 20 g SepaFlash Silica Flash Column, Eluent of 0˜10% Ethyl acetate/Petroleum ether gradient @100 mL/min), the eluent was concentrated to give Intermediate 1-3 (650 mg, 2.53 mmol, 80.60% yield, 97.571% purity) as a brown solid. LCMS (Method E): Rt=0.510 min, [M+H]⁺=251.0. ¹H NMR (400 MHz, CHLOROFORM-d) δ=8.85 (d, J=4.8 Hz, 2H), 8.10 (d, J=9.2 Hz, 2H), 7.31-7.28 (m, 1H), 3.99 (s, 3H). ¹⁹F NMR (377 MHz, CHLOROFORM-d) δ=−109.76 (s, 2F).

### Step 2: Synthesis of Intermediate 1-4

To the mixture of Intermediate 1-3 (650 mg, 2.60 mmol, 1 eq) in THF (2.5 mL), MeOH (2.5 mL) and H₂O (2.5 mL) was added LiOH·H₂O (327.05 mg, 7.79 mmol, 3 eq). The reaction mixture was stirred at 25° C. for 1 hr. To the reaction mixture was added 1 N HCl to adjust pH to 3 and off-white precipitate was formed, and then the mixture was filtered and the filter cake was collected and dissolved with MeOH (150 mL), and the solution was dried over anhydrous Na₂SO₄ and concentrated to give Intermediate 1-4 (600 mg, 2.39 mmol, 92.12% yield, 94.207% purity) as a light-yellow solid. LCMS (Method E): Rt=0.434 min, [M+H]⁺=237.2. ¹H NMR (400 MHz, METHANOL-d₄) δ=8.90 (d, J=4.8 Hz, 2H), 8.11 (d, J=9.2 Hz, 2H), 7.46-7.43 (m, 1H). ¹⁹F NMR (377 MHz, METHANOL-d₄) δ=−112.78 (s, 2F).

### Step 3: Synthesis of I-967

To the mixture of Intermediate 1-4 (190.10 mg, 804.90 μmol, 1 eq), Intermediate 1-5 (630.00 mg, 804.90 μmol, 1 eq, HCl salt), HOAt (109.56 mg, 804.90 μmol, 112.60 μL, 1 eq) and EDCI (462.90 mg, 2.41 mmol, 3 eq) in DMF (5 mL) was added NMM (651.33 mg, 6.44 mmol, 707.96 μL, 8 eq). The reaction mixture was stirred at 25° C. for 0.5 hr. The reaction mixture was combined with another lot of material and diluted with water (15 mL) and extracted with EA (10 mL*3), the combined organic phase was dried over anhydrous Na₂SO₄ and concentrated to give the residue. The residue was purified by reverse-phase chromatography (120 g of XB—C18, 20-40 μm, 120 Å) Mobile phase: A for H₂O (0.1% FA, v/v) and B for acetonitrile; Gradient: B5%-33% in 20 min; Flow rate: 40 ml/min; Column temperature: R.T. Wavelength: 220 nm/254 nm), and the eluent was lyophilized to give I-967 (373.34 mg, 456.87 μmol, 56.76% yield, 98.86% purity) as a white solid. LCMS (Method E): Rt=0.425 min, [M+H]⁺=808.3. ¹H NMR (400 MHz, METHANOL-d₄) δ=8.90 (d, J=4.8 Hz, 2H), 8.43 (s, 1H), 8.37 (d, J=7.6 Hz, 1H), 8.17-8.06 (m, 2H), 7.99-7.92 (m, 1H), 7.90-7.79 (m, 2H), 7.49 (s, 1H), 7.45-7.43 (m, 1H), 7.41-7.32 (m, 1H), 7.18-7.14 (m, 1H), 4.71 (d, J=12.0 Hz, 1H), 4.38 (s, 2H), 3.83-3.72 (m, 2H), 3.68 (br s, 2H), 3.62 (d, J=13.6 Hz, 1H), 3.51 (s, 2H), 3.40 (s, 1H), 3.33 (s, 3H), 3.24-3.18 (m, 1H), 2.96-2.90 (m, 1H), 2.67 (d, J=15.6 Hz, 8H), 2.49-2.34 (m, 2H), 1.95 (d, J=10.4 Hz, 2H), 1.83 (d, J=13.2 Hz, 1H), 1.33-1.10 (m, 2H). ¹⁹F NMR (377 MHz, METHANOL-d₄) δ=−115.10 (br d, J=26.9 Hz, 2F), −120.55-−120.90 (m, 1F).

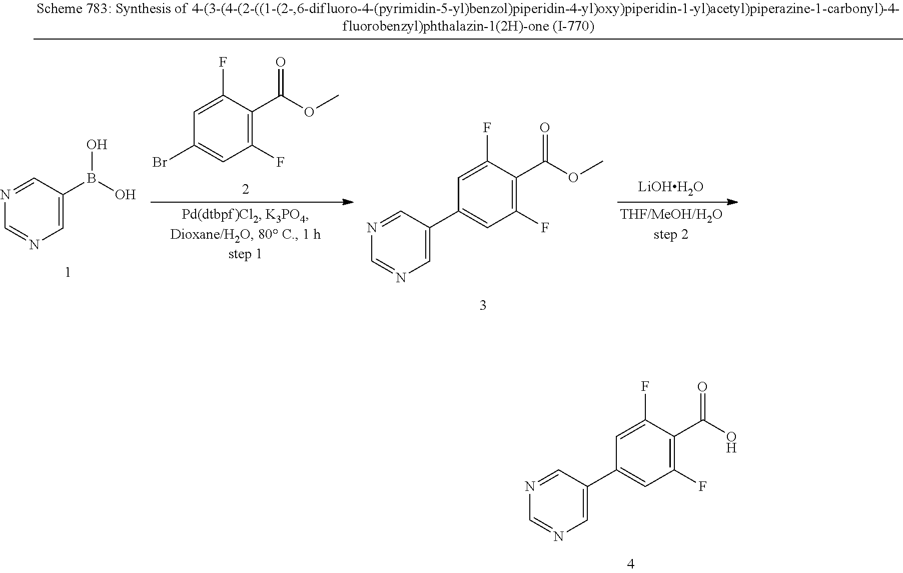

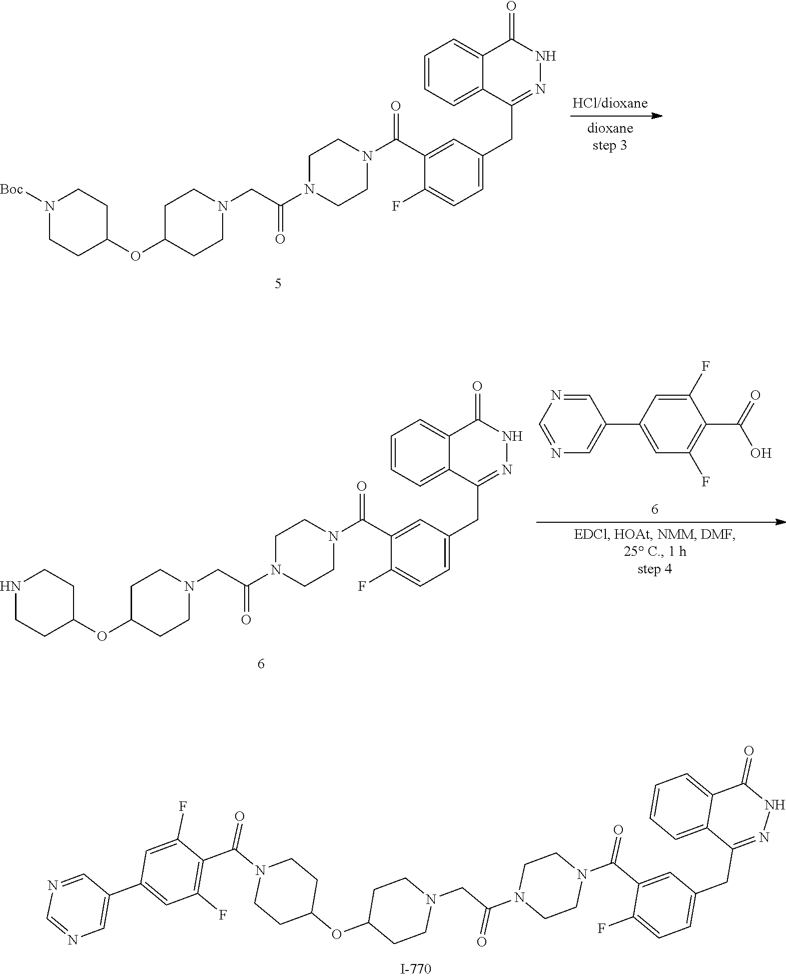

### Step 1: Synthesis of Intermediate 3

To a solution of Intermediate 1 (700 mg, 5.65 mmol, 1 eq) and Intermediate 2 (1.42 g, 5.65 mmol, 1 eq) in dioxane (7.5 mL) and H₂O (1.5 mL) was added Pd(dtbpf)Cl₂ (368.20 mg, 564.95 μmol, 0.1 eq) and K₃PO₄ (2.40 g, 11.30 mmol, 2 eq). The mixture was stirred at 80° C. for 1 hr. The mixture was diluted with water (12 mL) and extracted with DCM (12 mL*3). The combined organic layer was dried over Na₂SO₄, filtered and concentrated in vacuo to give a residue. The residue was purified by column chromatography (SiO₂, PE:EA=20:1 to PE:EA=0:1), then the organic liquid was concentrated in vacuo to give Intermediate 3 (500 mg, 1.74 mmol, 30.77% yield, 87% purity) as a yellow solid. LCMS (Method D): Rt=0.360 min, [M+H]⁺=251.0.

### Step 2: Synthesis of Intermediate 4

To a solution of Intermediate 3 (250 mg, 999.20 μmol, 1 eq) in THF (1 mL), MeOH (1 mL), and H₂O (1 mL) was added LiOH·H₂O (125.79 mg, 3.00 mmol, 3 eq). The mixture was stirred at 25° C. for 0.5 hr. The mixture was concentrated and lyophilized to give a crude product (350 mg, crude). Then to the crude product was added H₂O (5 mL) and HCl (1 M, 3 mL, 2.08 eq). The mixture was stirred at 25° C. for 0.5 hr. The mixture was filtered and the filter cake was dried to give Intermediate 4 (220 mg, 809.39 μmol, 55.99% yield, 86.89% purity) as a gray solid. LCMS (Method E): Rt=0.353 min, [M+H]⁺=237.1.

### Step 3: Synthesis of Intermediate 6

To a solution of Intermediate 5 (5 g, 7.24 mmol, 1 eq) in dioxane (25 mL) was added HCl/dioxane (4 M, 25 mL, 13.82 eq). The mixture was stirred at 25° C. for 1 hr. The reaction mixture was concentrated to give Intermediate 6 (5 g, 6.94 mmol, 95.83% yield, 87% purity, HCl salt) as a yellow solid. LCMS (Method E): Rt=0.363 min, [M+H]⁺=591.2. ¹H NMR (400 MHz, DMSO-d6) δ=12.61 (br d, J=4.8 Hz, 1H), 9.74 (m, 1H), 9.29-8.91 (m, 2H), 8.26 (br d, J=8.0 Hz, 1H), 8.00-7.93 (m, 1H), 7.93-7.86 (m, 1H), 7.86-7.78 (m, 1H), 7.51-7.43 (m, 1H), 7.36 (m, 1H), 7.25 (m, 1H), 4.53-4.25 (m, 4H), 3.82-3.67 (m, 3H), 3.48-3.39 (m, 3H), 3.30 (br d, J=11.2 Hz, 3H), 3.23-3.00 (m, 5H), 2.98-2.84 (m, 2H), 2.14-2.01 (m, 1H), 2.00-1.82 (m, 4H), 1.79-1.61 (m, 3H). ¹⁹F NMR (400 MHz, DMSO-d6) δ=−119.71.

### Step 4: Synthesis of I-770

To a solution of Intermediate 4 (200 mg, 846.83 μmol, 1 eq) and Intermediate 6 (531.09 mg, 846.83 μmol, 1 eq, HCl salt) in DMF (2 mL) was added HOAt (172.89 mg, 1.27 mmol, 177.69 μL, 1.5 eq), EDCI (324.68 mg, 1.69 mmol, 2 eq) and NMM (428.27 mg, 4.23 mmol, 465.51 μL, 5 eq). The mixture was stirred at 25° C. for 1 hr. The reaction was quenched by water (1 mL) to give a mixture. The mixture was purified by reversed phase HPLC (ISCO; 80 g SepaFlash C18 Column, Eluent of 0˜50% (0.1% FA) water/MeCN@60 mL/min). The eluent was collected and lyophilized to give the crude product. Then the crude product was purified by Prep-HPLC (column: Phenomenex luna C18 150*25 mm*10 um; mobile phase: [water(FA)-ACN]; gradient: 10%-40% B over 10 min). The eluent was concentrated and lyophilized to give I-770 (182 mg, 223.44 μmol, 26.39% yield, 99.3% purity) as a white solid. LCMS (Method E): Rt=0.416 min, [M+H]⁺=809.3. ¹H NMR (400 MHz, DMSO-d6) δ=12.59 (s, 1H), 9.25 (d, J=1.6 Hz, 3H), 8.26 (d, J=7.6 Hz, 1H), 8.15 (s, 1H), 8.00-7.93 (m, 1H), 7.89 (m, 1H), 7.86-7.75 (m, 3H), 7.44 (br s, 1H), 7.37 (m, 1H), 7.23 (m, 1H), 4.33 (s, 2H), 4.09-3.92 (m, 1H), 3.80-3.68 (m, 1H), 3.67-3.55 (m, 3H), 3.54 (br s, 5H), 3.23-3.01 (m, 6H), 2.75-2.60 (m, 2H), 2.24-2.04 (m, 2H), 1.93-1.67 (m, 4H), 1.52-1.26 (m, 4H). ¹⁹F NMR (400 MHz, DMSO-d6) δ=−113.26, −119.78.

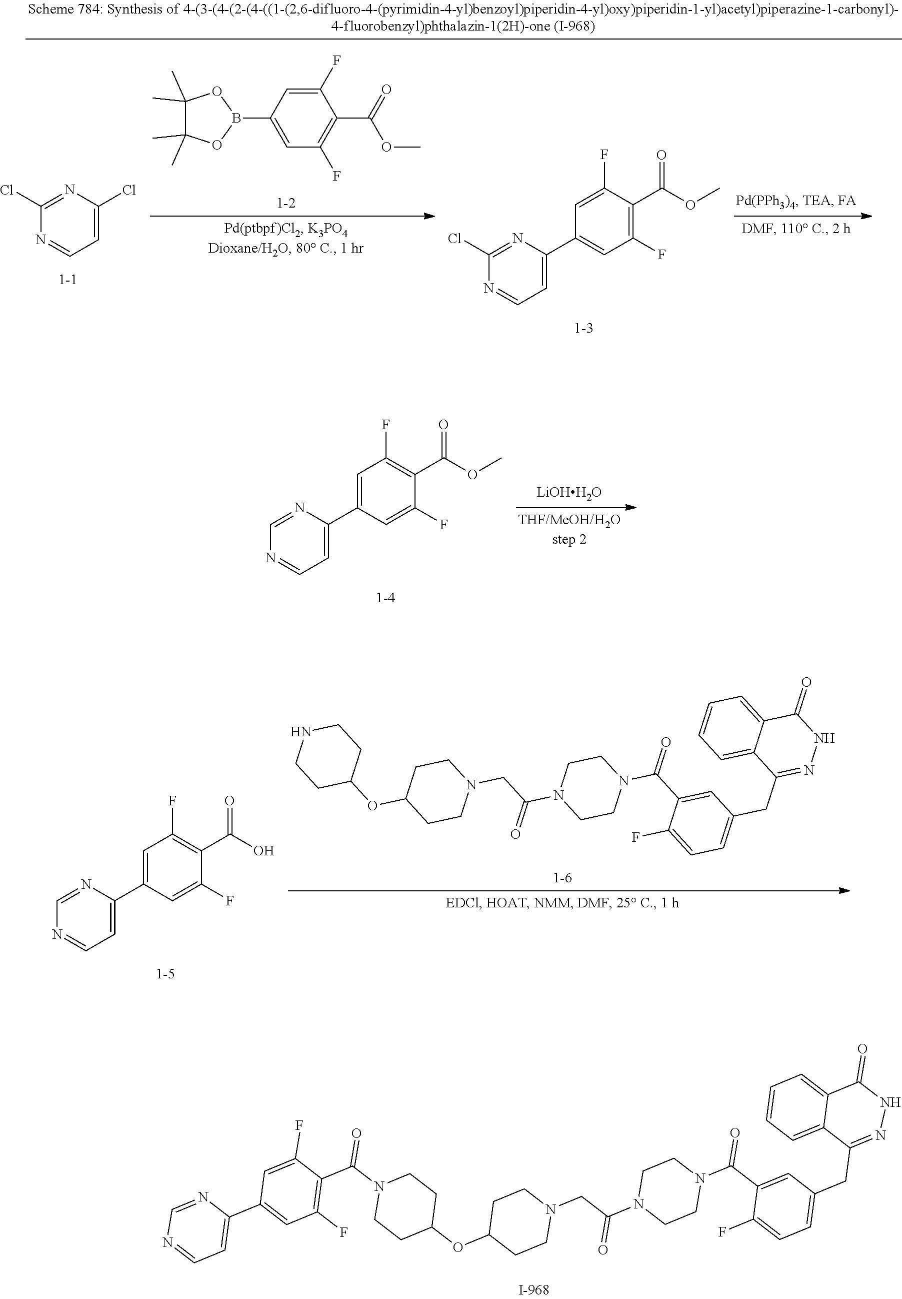

### Step 1: Synthesis of Intermediate 1-3

To a solution of Intermediate 1-1 (1.0 g, 6.71 mmol, 1 eq) and Intermediate 1-2 (1.00 g, 3.36 mmol, 0.5 eq) in dioxane (40 mL) and water (10 mL) was added K₃PO₄ (4.27 g, 20.14 mmol, 3.0 eq) and Pd(dppf)Cl₂ (491.15 mg, 671.24 μmol, 0.1 eq). The suspension was degassed and purged with N₂ 3 times. The mixture was stirred at 80° C. for 1 hr. The mixture was diluted with water (10 mL) and extracted with EA (10 mL*3). The organic layer was concentrated under vacuum. The residue was combined with another lot of material for purification. The residue was purified by flash silica gel chromatography (ISCO; 80 g SepaFlash Silica Flash Column, Eluent of 0˜20% Ethyl acetate/Petroleum ether gradient @80 mL/min) and concentrated under vacuum. Intermediate 1-3 (0.9 g, 3.06 mmol, 45.59% yield, 96.79% purity) was obtained as a white solid. LCMS (Method E): Rt=0.547 min, [M+H]⁺=285.1. ¹H NMR (400 MHz, CHLOROFORM-d) δ=8.77 (d, J=5.2 Hz, 1H), 7.73 (d, J=8.8 Hz, 2H), 7.64 (d, J=5.2 Hz, 1H), 4.00 (s, 3H). ¹⁹F NMR (377 MHz, CHLOROFORM-d) δ=−107.95 (s, 1F).

### Step 2: Synthesis of Intermediate 1-4

To a solution of Intermediate 1-3 (900 mg, 3.16 mmol, 1 eq) in DMF (20 mL) was added Pd(PPh₃)₄ (365.37 mg, 316.18 μmol, 0.1 eq), formic acid (291.08 mg, 6.32 mmol, 2.0 eq) and TEA (319.95 mg, 3.16 mmol, 1.0 eq). The suspension was degassed and purged with N₂ for 3 times. The mixture was stirred at 110° C. for 2 hr under N₂. The mixture was diluted with water (10 mL) and extracted with EA (10 mL*3), dried over Na₂SO₄, filtered and concentrated under reduced pressure to give a residue. The residue was purified by flash silica gel chromatography (ISCO; 20 g SepaFlash Silica Flash Column, Eluent of 0˜30% Ethyl acetate/Petroleum ethergradient @80 mL/min) and concentrated under vacuum. Intermediate 1-4 (600 mg, 2.40 mmol, 75.84% yield, 100% purity) was obtained as a yellow solid. LCMS (Method E): Rt=0.453 min, [M+H]⁺=251.1. ¹H NMR (400 MHz, CHLOROFORM-d) δ=9.33 (s, 1H), 8.88 (d, J=5.2 Hz, 1H), 7.77-7.68 (m, 3H), 4.00 (s, 3H).

### Step 3: Synthesis of Intermediate 1-5

To a solution of Intermediate 1-4 (500 mg, 2.00 mmol, 1 eq) in THF (4 mL), MeOH (4 mL) and water (2 mL) was added LiOH·H₂O (167.72 mg, 4.00 mmol, 2.0 eq), and the mixture was stirred at 25° C. for 1 hr. The reaction mixture was combined with another lot of material (100 mg) to work up. The combined mixture was concentrated under vacuum to remove MeOH, then adjusted to pH=3-4 with aq. HCl (1 M), some solid formed. The mixture was filtered and the filter cake was collected. The residue was triturated with water (5 mL) for 0.5 h, filtered and the filter cake was collected and dried under vacuum. Compound Intermediate 1-5 (450 mg, 1.91 mmol, 95.35% yield) was obtained as off-white solid. H NMR (400 MHz, DMSO-d6) δ=9.33 (d, J=1.2 Hz, 1H), 8.98 (d, J=5.6 Hz, 1H), 8.26-8.23 (m, 1H), 8.05 (d, J=9.2 Hz, 2H). ¹⁹F NMR (376 MHz, DMSO-d6) δ=−110.86 (s, 1F).

### Step 4: Synthesis of I-968

To a solution of Intermediate 1-5 (150 mg, 635.12 μmol, 1 eq) in DMF (6.0 mL) was added EDCI (365.26 mg, 1.91 mmol, 3.0 eq) and HOAt (129.67 mg, 952.69 μmol, 1.5 eq) and 4-[[4-fluoro-3-[4-[2-[4-(4-piperidyloxy)-1-piperidyl]acetyl]piperazine-1-carbonyl]phenyl]methyl]-2H-phthalazin-1-one (375.16 mg, 598.20 μmol, 9.42e−1 eq, HCl salt) and NMM (321.20 mg, 3.18 mmol, 349.14 μL, 5.0 eq). The mixture was stirred at 25° C. for 1 hr. The reaction mixture was combined with another lot of material (30 mg) to work up and for purification. The crude product was purified by reverse phase chromatography (100 g of XB—C18, 20-40 μm, 120 Å) Mobile phase: A for H₂O (0.1% FA v/v) and B for acetonitrile; Gradient: B 5%-22% in 15 min; Flow rate: 80 ml/min; Column temperature: R.T. Wavelength: 220 nm/254 nm) and was dried by lyophilization. I-968 (187.23 mg, 229.39 μmol, 36.12% yield, 99.10% purity) was obtained as a white solid. LCMS (Method E): Rt=0.441 min, [M+H]⁺=809.4. ¹H NMR (400 MHz, DMSO-d6) δ=12.56 (s, 1H), 9.32 (s, 1H), 8.98 (d, J=5.6 Hz, 1H), 8.30-8.20 (m, 2H), 8.14 (s, 1H), 8.07 (d, J=8.8 Hz, 2H), 7.99-7.93 (m, 1H), 7.92-7.80 (m, 2H), 7.47-7.32 (m, 2H), 7.23 (t, J=9.2 Hz, 1H), 4.33 (s, 2H), 4.08-3.94 (m, 1H), 3.77-3.54 (m, 4H), 3.50-3.35 (m, 6H), 3.20-3.07 (m, 5H), 2.74-2.60 (m, 2H), 2.24-2.06 (m, 2H), 1.92-1.70 (m, 4H), 1.54-1.28 (m, 4H). ¹⁹F NMR (377 MHz, DMSO-d6) δ=−113.10 (t, J=7.9 Hz, 2F), −119.79 (s, 1F).

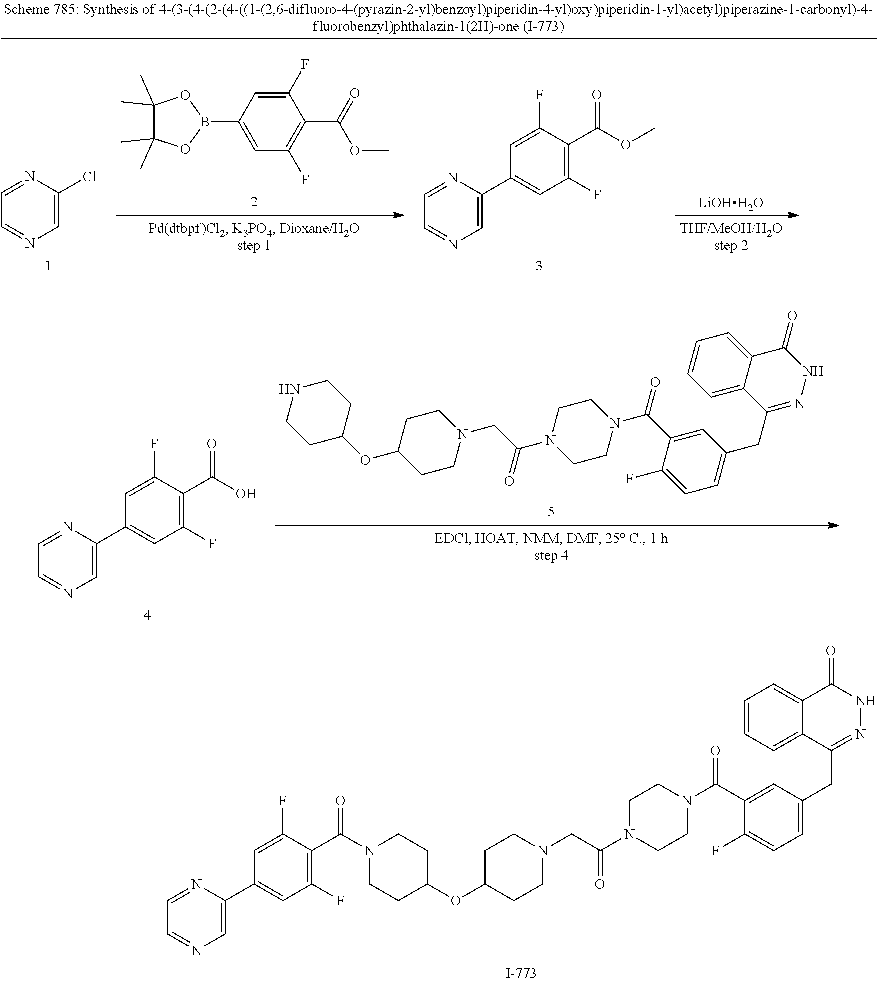

### Step 1: Synthesis of Intermediate 3

A mixture of Intermediate 1 (200 mg, 1.75 mmol, 155.88 μL, 1 eq), Intermediate 2 (520.53 mg, 1.75 mmol, 1 eq), Pd(dppf)Cl₂ (113.81 mg, 174.62 μmol, 0.1 eq) and K₃PO₄ (1.11 g, 5.24 mmol, 3 eq) in dioxane (5 mL) and H₂O (1 mL) was degassed and purged with N₂ 3 times, and then the mixture was stirred at 80° C. for 1 hr under N₂ atmosphere. The reaction mixture was combined with another batch of material for workup, and the resulting mixture was poured into water (10 mL) and extracted with EA (8 mL*3). The combined organic layers were dried over Na₂SO₄, filtered and concentrated under reduced pressure to give a residue. The residue was purified by flash silica gel chromatography (ISCO; 20 g SepaFlash Silica Flash Column, Eluent of 0˜100% PE/EA)@50 mL/min, PE/EA=2:1, Rf=0.4) and the eluent was concentrated under reduced pressure to give Intermediate 3 (450 mg, crude) as a yellow solid. LCMS (Method D): Rt=0.369 min, [M+H]⁺=251.0.

### Step 2: Synthesis of Intermediate 4

To a solution of Intermediate 3 (350 mg, 1.40 mmol, 1 eq) in THF (1.5 mL), MeOH (1.5 mL) and H₂O (1.5 mL) was added LiOH·H₂O (176.11 mg, 4.20 mmol, 3 eq). The mixture was stirred at 25° C. for 0.5 hr. The reaction mixture was combined with another batch of material for workup. The reaction pH was adjusted to pH=3 with 1M HCl, and the precipitate was collected and dried under reduced pressure to give Intermediate 4 (400 mg, crude) as a yellow solid. LCMS (Method D): Rt=0.269 min, [M+H]⁺=237.0.

### Step 4: Synthesis of I-773

To a solution of Intermediate 4 (200 mg, 846.83 μmol, 1 eq) and Intermediate 5 (531.09 mg, 846.83 μmol, 1 eq, HCl salt) in DMF (2 mL) was added HOAt (172.89 mg, 1.27 mmol, 177.69 μL, 1.5 eq), EDCI (324.68 mg, 1.69 mmol, 2 eq) and NMM (428.27 mg, 4.23 mmol, 465.51 μL, 5 eq). The mixture was stirred at 25° C. for 1 hr. The reaction was quenched by water (1 mL). The mixture was purified by reversed phase HPLC (ISCO; 80 g SepaFlash C18 Column, Eluent of 0˜50% (0.1% FA) water/MeCN@60 mL/min). The collected eluent was concentrated and lyophilized to give a crude product. Then the crude product was purified by Prep-HPLC (column: Unisil C8; mobile phase: [water (FA)-ACN]; gradient: 10%-40% B over 40 min). The eluent was collected and lyophilized to give a crude product. Then the crude product was purified by Prep-HPLC (column: Waters Xbridge 150*25 mm*5 um; mobile phase: [water (NH₄HCO₃)-ACN]; gradient: 18%-48% B over 15 min). The eluent was lyophilized to give I-773 (161 mg, 198.39 mol, 23.43% yield, 99.67% purity) as a yellow solid. LCMS (Method E): Rt=0.426 min, [M+H]⁺=809.3. ¹H NMR (400 MHz, DMSO-d6) δ=12.59 (s, 1H), 9.39 (d, J=1.2 Hz, 1H), 8.84-8.75 (m, 1H), 8.72 (d, J=2.4 Hz, 1H), 8.26 (d, J=7.6 Hz, 1H), 8.01 (br d, J=8.8 Hz, 2H), 7.98-7.93 (m, 1H), 7.92-7.86 (m, 1H), 7.86-7.79 (m, 1H), 7.49-7.40 (m, 1H), 7.40-7.32 (m, 1H), 7.23 (m, 1H), 4.33 (s, 2H), 4.10-3.96 (m, 1H), 3.79-3.67 (m, 1H), 3.67-3.53 (m, 3H), 3.53-3.38 (m, 5H), 3.25-3.01 (m, 6H), 2.74-2.59 (m, 2H), 2.18-2.04 (m, 2H), 1.91-1.69 (m, 4H), 1.52-1.28 (m, 4H). F NMR (400 MHz, DMSO-d6) δ=−113.2, −119.8.

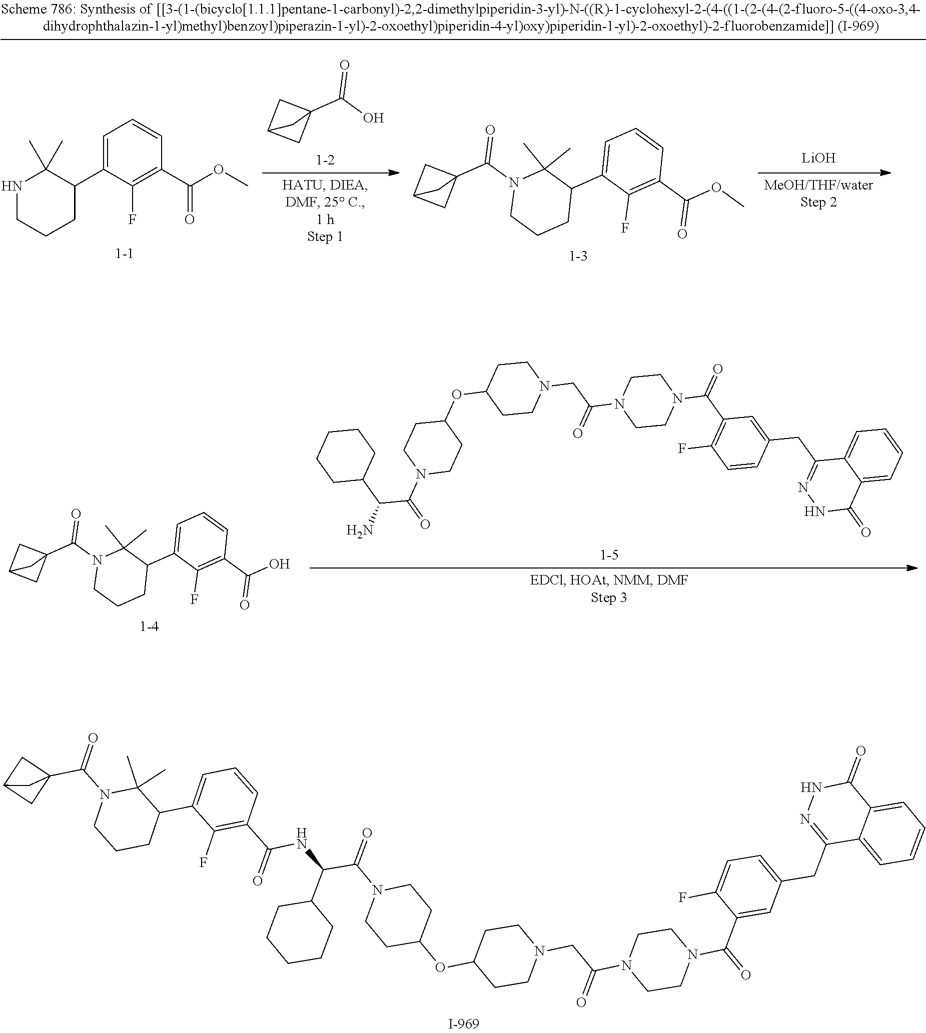

### Step 1: Synthesis of Intermediate 1-3

To a solution of Intermediate 1-1 (250 mg, 942.25 μmol, 1 eq) and Intermediate 1-2 (126.78 mg, 1.13 mmol, 1.2 eq) in DMF (3 mL) was added HATU (537.41 mg, 1.41 mmol, 1.5 eq) and DIEA (365.34 mg, 2.83 mmol, 492.37 μL, 3 eq). The reaction mixture was stirred at 25° C. for 1 hr. The reaction mixture was diluted with water (5 mL) and extracted with EA (5 mL*4). The combined organic phase was dried by anhydrous Na₂SO₄, filtered and concentrated under vacuum to give a residue. The residue was purified by flash silica gel chromatography (ISCO; 20 g SepaFlash Silica Flash Column, Eluent of 0˜50% Ethyl acetate/Petroleum ether gradient @60 mL/min) and concentrated under vacuum to give Intermediate 1-3 (330 mg, 890.57 μmol, 94.52% yield, 97% purity) as a yellow oil. LCMS (Method E): Rt=0.597 min, [M+H]⁺=360.3. ¹H NMR (400 MHz, CHLOROFORM-d) δ=7.83-7.75 (m, 1H), 7.49-7.43 (m, 1H), 7.15 (t, J=7.8 Hz, 1H), 3.93 (s, 3H), 3.79-3.70 (m, 1H), 3.63-3.55 (m, 1H), 3.53-3.42 (m, 1H), 2.49 (s, 1H), 2.18-2.14 (m, 6H), 2.10-1.97 (m, 1H), 1.91-1.80 (m, 1H), 1.77-1.65 (m, 2H), 1.46 (d, J=2.3 Hz, 3H), 1.37 (s, 3H). ¹⁹F NMR (376 MHz, CHLOROFORM-d) δ=−111.68-−114.08 (m, 1F).

### Step 2: Synthesis of Intermediate 1-4

To a solution of Intermediate 1-3 (300 mg, 834.65 μmol, 1 eq) in THF (1.5 mL), MeOH (0.7 mL) and H₂O (1.5 mL) was added LiOH·H₂O (105.07 mg, 2.50 mmol, 3 eq). The reaction mixture was stirred at 25° C. for 0.5 hr. The reaction mixture was adjusted to pH=3 and extracted with EA (10 mL*3). The combined organic phase was dried by anhydrous Na₂SO₄, filtered and concentrated to give Intermediate 1-4 (280 mg, 778.21 μmol, 93.24% yield, 96% purity) as a white solid. LCMS (Method E): Rt=0.532 min, [M+H]⁺=346.3. ¹H NMR (400 MHz, CHLOROFORM-d) δ=7.95-7.85 (m, 1H), 7.55-7.48 (m, 1H), 7.19 (t, J=7.8 Hz, 1H), 3.80-3.70 (m, 1H), 3.61 (td, J=5.4, 13.9 Hz, 1H), 3.56-3.44 (m, 1H), 2.49 (s, 1H), 2.19-2.13 (m, 6H), 2.11-2.00 (m, 1H), 1.92-1.81 (m, 1H), 1.78-1.67 (m, 2H), 1.47 (d, J=2.1 Hz, 3H), 1.39 (s, 3H). ¹⁹F NMR (376 MHz, CHLOROFORM-d) δ=−109.24-−114.48 (m, 1F).

### Step 3: Synthesis of I-969

To a solution of Intermediate 1-4 (180 mg, 521.12 μmol, 1 eq) and Intermediate 1-5 (479.23 mg, 625.35 μmol, 1.2 eq, HCl salt) in DMF (2 mL) was added HOAt (70.93 mg, 521.12 μmol, 72.90 μL, 1 eq), EDCI (199.80 mg, 1.04 mmol, 2 eq) and NMM (263.55 mg, 2.61 mmol, 286.47 μL, 5 eq). The reaction mixture was stirred at 25° C. for 1 hr. The reaction mixture was diluted with water (6 mL), extracted with EA (10 mL*3) and washed with brine (10 mL*2). The combined organic phase was dried by anhydrous Na₂SO₄, filtered and concentrated under vacuum to give a residue. The residue was purified by prep-HPLC (column: Waters Xbridge Prep OBD C18 150*40 mm*10 um; mobile phase: [water (NH₄HCO₃)-ACN]; gradient: 45%-75% B over 58 min), and then concentrated to remove MeCN and lyophilized to give the product (HPLC purity: 98%). The product was purified by prep-HPLC (column: Phenomenex luna C18 150*40 mm*15 um; mobile phase: [water (FA)-ACN]; gradient: 30%-60% B over 15 min) and freeze-dried. I-969 (330.41 mg, 298.34 μmol, 60.66% yield, 99.62% purity, FA salt) was obtained as a white solid. LCMS (Method G): Rt=0.735&0.755 min, [M+H]⁺=1057.6. SFC: Retention time=3.198 min and 3.806 min. ¹H NMR (400 MHz, METHANOL-d4) δ=8.47 (s, 1H), 8.37 (br d, J=7.6 Hz, 1H), 7.95 (br d, J=6.8 Hz, 1H), 7.92-7.81 (m, 2H), 7.60-7.48 (m, 3H), 7.42-7.35 (m, 1H), 7.29-7.21 (m, 1H), 7.21-7.14 (m, 1H), 4.99-4.94 (m, 1H), 4.39 (s, 2H), 4.12-4.01 (m, 1H), 3.95-3.45 (m, 17H), 3.15-3.02 (m, 2H), 2.88-2.69 (m, 2H), 2.47 (s, 1H), 2.17 (s, 7H), 2.04-1.64 (m, 17H), 1.57-1.49 (m, 1H), 1.43 (br s, 3H), 1.38 (s, 3H), 1.32-1.09 (m, 5H). ¹⁹F NMR (377 MHz, METHANOL-d4) δ=−116.84-−119.55 (m, 1F), −120.68 (br d, J=20.2 Hz, 1F).

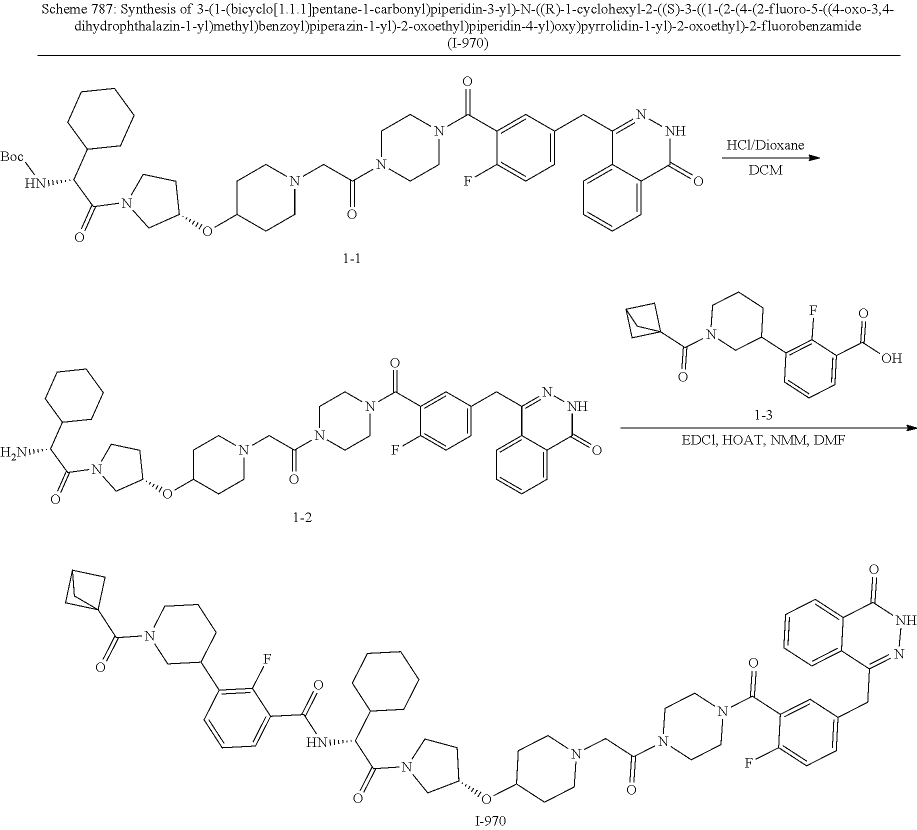 [SMILES: CC(C)(C)OC(=O)N[C@@H](C(=O)N1CC[C@H](OC2CCN(CC(=O)N3CCN(C(=O)c4cc(Cc5n[nH]c(=O)c6ccccc56)ccc4F)CC3)CC2)C1)C1CCCCC1 ; N[C@@H](C(=O)N1CC[C@H](OC2CCN(CC(=O)N3CCN(C(=O)c4cc(Cc5n[nH]c(=O)c6ccccc56)ccc4F)CC3)CC2)C1)C1CCCCC1 ; O=C(O)c1cccc(C2CCCN(C(=O)C34CC(C3)C4)C2)c1F ; O=C(N[C@@H](C(=O)N1CC[C@H](OC2CCN(CC(=O)N3CCN(C(=O)c4cc(Cc5n[nH]c(=O)c6ccccc56)ccc4F)CC3)CC2)C1)C1CCCCC1)c1cccc(C2CCCN(C(=O)C34CC(C3)C4)C2)c1F]

### Step 1: Synthesis of Intermediate 1-2

To a solution of Intermediate 1-1 (800 mg, 980.43 μmol, 1 eq) in DCM (4 mL) was added HCl/Dioxane (4 M, 4.00 mL). The mixture was stirred at 25° C. for 1 hr. The mixture was concentrated under vacuum to give Intermediate 1-2 (700 mg, crude, HCl salt) as a white solid. LCMS (Method G): Rt=0.543 min, [M+H]⁺=716.5.

### Step 2: Synthesis of I-970

To a mixture of Intermediate 1-2 (0.3 g, 398.77 μmol, 1 eq, HCl salt), Intermediate 1-3 (126.55 mg, 398.77 μmol, 1 eq) and HOAt (54.28 mg, 398.77 μmol, 55.78 μL, 1 eq) in DMF (3 mL) was added EDCI (152.89 mg, 797.54 μmol, 2 eq) and NMM (201.68 mg, 1.99 mmol, 219.21 μL, 5 eq). The mixture was stirred at 25° C. for 2 hrs. The reaction mixture was combined with another lot of material for work up. The mixture was diluted with water (10 mL) and extracted with EtOAc (20 mL*3). The organic layer was concentrated under vacuum to give crude product. The crude product was purified by reversed-phase HPLC (0.1% NH₃·H₂O condition), and the eluent was concentrated under vacuum to remove MeCN and lyophilized. The product was re-purified by Prep-HPLC (column: Waters Xbridge Prep OBD C18 150*40 mm*10 um; mobile phase: [water (NH₄HCO₃)-ACN]; gradient: 32%-62% B over 15 min) and dried by lyophilization, The product was re-purified by normal phase HPLC (column: Welch Ultimate XB—SiOH 250*50*10 um; mobile phase: [Hexane-EtOH]; gradient: 20%-60% B over 15 min), and the eluent was concentrated and lyophilized to give I-970 (167.38 mg, 164.88 μmol, 41.35% yield, 100% purity) as an off-white solid. LCMS (Method x): Rt=16.518, 16.828 min, [M+H]⁺=1015.9. SFC: Rt=2.006, 2.716 min. ¹H NMR (400 MHz, METHANOL-d4) δ=8.41-8.32 (m, 1H), 7.98-7.91 (m, 1H), 7.91-7.78 (m, 2H), 7.59-7.45 (m, 3H), 7.37 (br d, J=5.6 Hz, 1H), 7.25-7.18 (m, 1H), 7.16 (dt, J=3.0, 8.8 Hz, 1H), 4.72-4.61 (m, 1H), 4.53 (br d, J=13.2 Hz, 1H), 4.43-4.26 (m, 4H), 4.03 (br d, J=10.4 Hz, 1H), 3.84-3.62 (m, 5H), 3.60-3.45 (m, 5H), 3.25 (br s, 1H), 3.21-3.11 (m, 2H), 3.09-2.93 (m, 1H), 2.83-2.62 (m, 3H), 2.52-2.40 (m, 1H), 2.34-2.15 (m, 8H), 2.12-2.10 (m, 1H), 2.05-1.94 (m, 3H), 1.93-1.74 (m, 7H), 1.70-1.69 (m, 2H), 1.65-1.49 (m, 3H), 1.35-1.22 (m, 4H), 1.20-1.08 (m, 2H), 0.91-0.90 (m, 1H). ¹⁹F NMR (377 MHz, METHANOL-d4) δ=−120.76 (br d, J=6.7 Hz, 1F), −121.15-−121.84 (m, 1F).

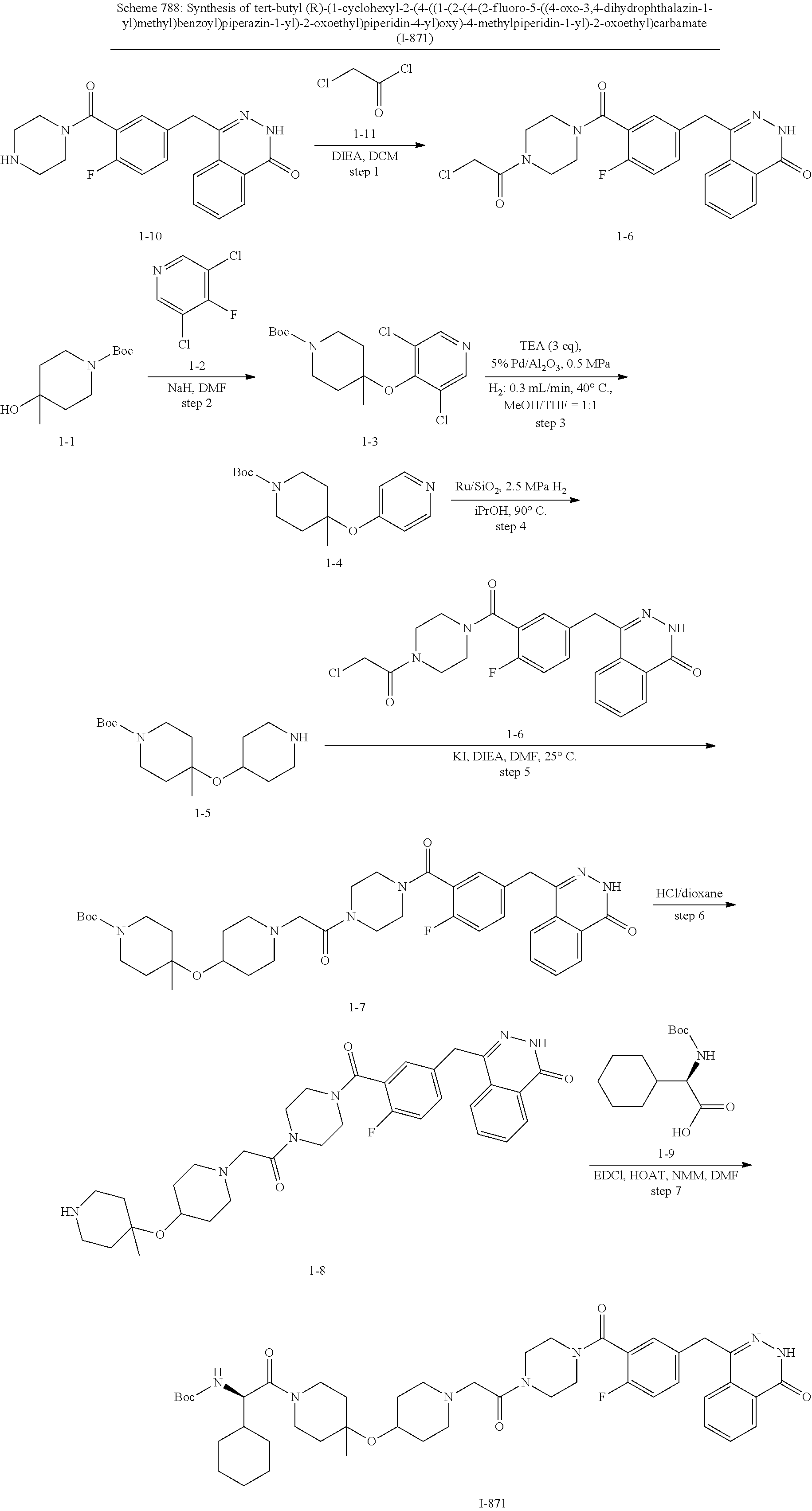

### Step 1: Synthesis of Intermediate 1-6

To a solution of Intermediate 1-10 (10 g, 27.29 mmol, 1 eq) in DCM (100 mL) was added DIEA (10.58 g, 81.88 mmol, 14.26 mL, 3 eq), and Intermediate 1-11 (6.17 g, 54.59 mmol, 4.35 mL, 2 eq). The mixture was stirred at 0-25° C. for 1 h. The reaction was poured into water (200 ml), extracted with DCM (100 ml*3), the combined organic layers were washed with sat. aq. NaHCO₃ (100 ml*3), dried over anhydrous Na₂SO₄, filtered and concentrated under vacuum to give a residue. The residue was purified by Prep-HPLC (column: Kromasil Eternity XT 250*80 mm*10 um; mobile phase: [water (ammonia hydroxide v/v)-ACN]; gradient: 20%-45% B over 20 min) and concentrated under vacuum to remove ACN. The aqueous was extracted with DCM (300 ml*3), the combined organic layers were dried over anhydrous Na₂SO₄, filtered and concentrated under vacuum. Intermediate 1-6 (7.5 g, 14.60 mmol, 53.48% yield, 86.19% purity) was obtained as a gray solid. LCMS (Method G): Retention time: 0.470 min, [M+H]⁺=443.2. ¹H NMR (400 MHz, DMSO-d6) ¹H NMR (400 MHz, DMSO-d6) δ=12.60 (s, 1H), 8.26 (dd, J=1.0, 7.8 Hz, 1H), 7.99-7.94 (m, 1H), 7.93-7.80 (m, 2H), 7.51-7.35 (m, 2H), 7.23 (t, J=9.0 Hz, 1H), 4.45-4.29 (m, 4H), 3.73-3.52 (m, 4H), 3.39 (br s, 2H), 3.21 (br d, J=16.1 Hz, 2H).

### Step 2: Synthesis of Intermediate 1-3

To a solution of Intermediate 1-1 (8.7 g, 40.41 mmol, 1 eq) in DMF (70 mL) was added NaH (3.23 g, 80.82 mmol, 60% purity, 2 eq) slowly at 0° C. After addition, the mixture was warmed to 25° C. and stirred at this temperature for 1 hr, and then Intermediate 1-2 (10.06 g, 60.62 mmol, 1.5 eq) in DMF (20 mL) was added at 25° C. The resulting mixture was stirred at 25° C. for 2 hr. The reaction mixture was quenched with NH₄Cl (100 mL) and extracted with EA (50 mL*3). The combined organic layers were washed with brine (50 mL*3), dried over anhydrous Na₂SO₄, filtered and concentrated under reduced pressure to give a residue. The residue was purified by flash silica gel chromatography (ISCO; 120 g SepaFlash Silica Flash Column, Eluent of 0˜20% Ethyl acetate/Petroleum ether gradient @100 mL/min) and concentrated under vacuum. Intermediate 1-3 (9.5 g, 26.30 mmol, 65.07% yield) was obtained as white solid. ¹H NMR (400 MHz, CHLOROFORM-d) δ=8.45 (s, 2H), 3.82 (br d, J=12.0 Hz, 2H), 3.55 (br t, J=11.9 Hz, 2H), 2.38 (br d, J=13.3 Hz, 2H), 1.75-1.61 (m, 2H), 1.48 (s, 9H), 1.25 (s, 3H).

### Step 3: Synthesis of Intermediate 1-4

A solution of Intermediate 1-3 (8.5 g, 23.53 mmol, 1 eq) and TEA (2.38 g, 23.53 mmol, 3.27 mL, 1 eq) in MeOH (85 mL) was stirred at 25° C. until a clear solution formed. A fixed bed (volume: 20 mL) was completely packed with granular catalyst 5% Pd/Al₂O₃ (1.00 eq, 10 g). The H₂ back pressure regulator was adjusted to 0.5 MPa, and the flow rate of H₂ was 60 mL/min. Then the solution was pumped (1.212 mL/min) to the fixed bed (9.525(½″) mm, 4 mL, 55° C.). The reaction mixture was collected from the reactor output. After the reaction was finished, the tubing was washed with MeOH (100 mL). The organics were concentrated under vacuum to give Intermediate 1-4 (7.6 g, crude) as a white solid. ¹H NMR (400 MHz, DMSO-d6) δ=8.45-8.38 (m, 2H), 7.10-7.05 (m, 2H), 3.69-3.61 (m, 2H), 3.22-3.13 (m, 2H), 2.02 (brd, J=13.8 Hz, 2H), 1.70-1.63 (m, 2H), 1.49 (s, 3H), 1.45-1.43 (m, 9H).

### Step 4: Synthesis of Intermediate 1-5

A solution of Intermediate 1-4 (7.6 g, 25.99 mmol, 1 eq) in IPA (76 mL) and THF (114 mL) was dissolved to form a clear solution. A fixed bed (volume: 20 mL) was packed with granular catalyst Ru/SiO₂ (10 g, 10% purity). The H₂ back pressure regulator was adjusted to 2.5 MPa and the flow rate of H₂ was 60 mL/min. Then the solution was pumped (1 mL/min) to the fixed bed (12.7 mm, 4 mL, 100° C.). Then the reaction mixture was collected from the reactor output. After the reaction fluid injection completed, the fixed bed was washed by IPA (114 mL). Intermediate 1-5 (7 g, crude) was obtained as a white solid. ¹H NMR (400 MHz, DMSO-d6) δ=3.88-3.77 (m, 1H), 3.52 (br d, J=13.0 Hz, 2H), 3.15-3.02 (m, 4H), 2.99-2.92 (m, 2H), 1.89-1.77 (m, 2H), 1.72-1.48 (m, 5H), 1.38 (s, 11H), 1.14 (s, 3H).

### Step 5: Synthesis of Intermediate 1-7

To a solution of Intermediate 1-5 (0.8 g, 2.68 mmol, 1 eq) in DMF (8 mL) was added Intermediate 1-6 (1.19 g, 2.68 mmol, 1 eq), DIEA (1.39 g, 10.72 mmol, 1.87 mL, 4 eq) and KI (1.78 g, 10.72 mmol, 4 eq). The mixture was stirred at 25° C. for 1 hr. The mixture was filtered. The filtrate was purified by reversed-phase HPLC (0.1% NH₃·H₂O). Intermediate 1-7 (1.3 g, 1.81 mmol, 67.59% yield, 98.237% purity) was obtained as a light yellow solid. LCMS (Method G): Retention time: 0.642 min, [M+H]⁺=705.5.

### Step 6: Synthesis of Intermediate 1-8

To a solution of Intermediate 1-7 (1 g, 1.42 mmol, 1 eq) in DCM (5 mL) was added HCl/dioxane (4 M, 5.00 mL, 14.10 eq). The mixture was stirred at 25° C. for 0.5 h. The mixture was concentrated under vacuum to give Intermediate 1-8 (0.9 g, crude, HCl salt) as a white solid. LCMS (Method G): Retention time: 0.775 min, [M+H]⁺=605.3.

### Step 7: Synthesis of I-871

To a solution of Intermediate 1-9 (361.20 mg, 1.40 mmol, 1 eq) in DMF (10 mL) was added HOAt (191.06 mg, 1.40 mmol, 196.36 μL, 1 eq), EDCI (538.17 mg, 2.81 mmol, 2 eq), NMM (709.89 mg, 7.02 mmol, 771.62 μL, 5 eq) and Intermediate 1-8 (0.9 g, 1.40 mmol, 1 eq, HCl salt). The mixture was stirred at 25° C. for 1 h. The mixture was poured into H₂O (30 mL), filtered and the filter cake was washed with H₂O (10 mL*3) and concentrated under vacuum to give crude I-871. Then 150 mg of the crude was dissolved in MeOH (2 mL) purified by Prep-HPLC (column: Waters Xbridge 150*25 mm*5 um; mobile phase: [water(NH₄HCO₃)-ACN]; gradient: 388%-68% B over 15 min). I-871 (89.39 mg, 104.64 μmol, 58.88% yield, 98.80% purity) was obtained as a white solid. LCMS (Method G): Retention time=0.685 min, [M+H]⁺=844.6. SFC: Retention time=1.817 min. ¹H NMR (400 MHz, DMSO-d6) δ=12.60 (s, 1H), 8.26 (d, J=7.6 Hz, 1H), 8.01-7.94 (m, 1H), 7.91-7.80 (m, 2H), 7.46-7.35 (m, 2H), 7.25-7.23 (m, 1H), 6.75-6.73 (m, 1H), 4.33 (s, 2H), 4.21 (br d, J=6.4 Hz, 1H), 3.98-3.44 (m, 9H), 3.20-2.99 (m, 5H), 2.67 (br d, J=2.0 Hz, 2H), 2.17-2.05 (m, 2H), 1.70-1.55 (m, 9H), 1.50-1.26 (m, 15H), 1.14 (br d, J=15.2 Hz, 6H), 0.98-0.87 (m, 2H). ¹⁹F NMR (377 MHz, DMSO-d6) δ=−118.06-−121.37 (m, 1F).

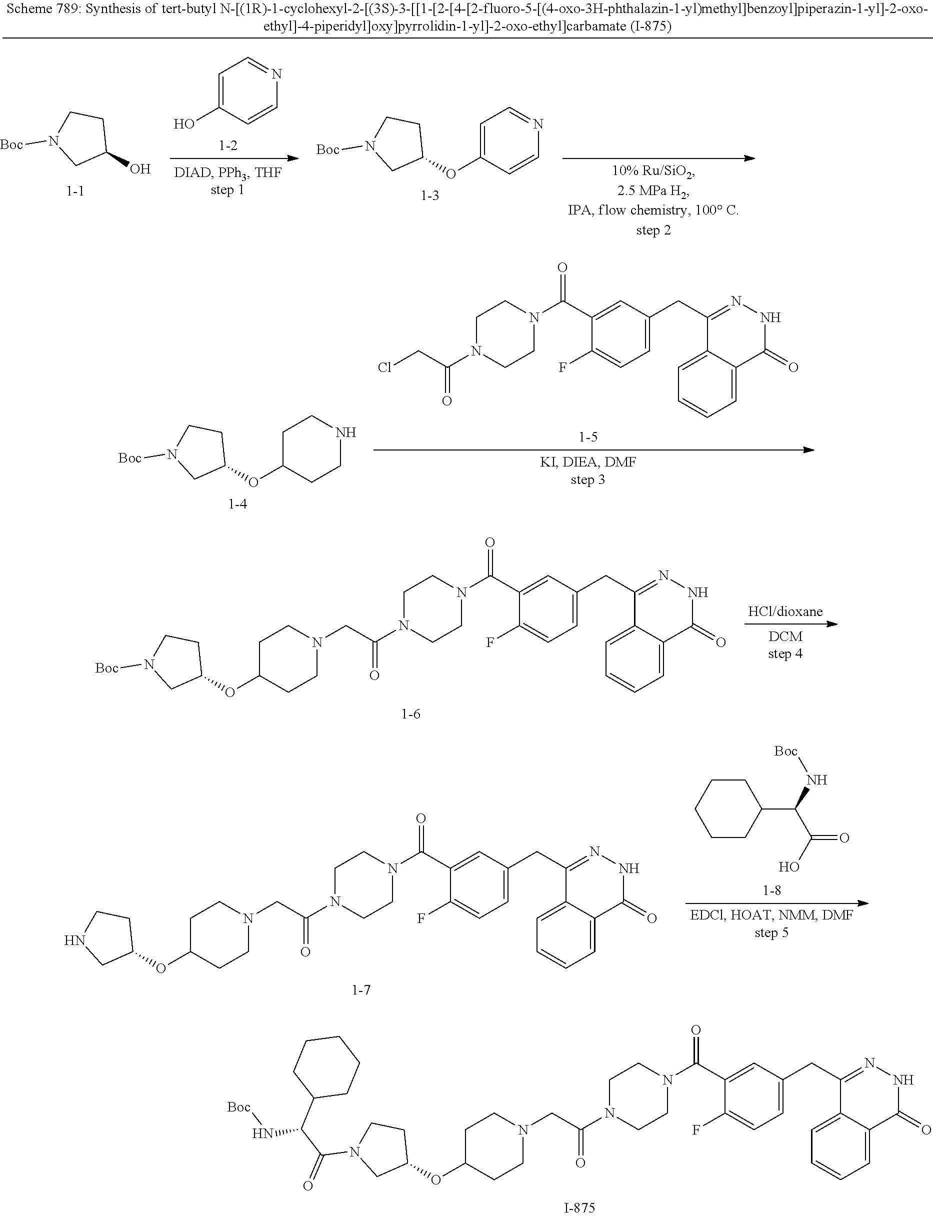

### Step 1: Synthesis of Intermediate 1-3

To a solution of Intermediate 1-1 (5 g, 26.70 mmol, 1 eq), Intermediate 1-2 (3.05 g, 32.05 mmol, 1.2 eq) and DIAD (10.80 g, 53.41 mmol, 10.35 mL, 2 eq) in THF (50 mL) was added PPh₃ (14.01 g, 53.41 mmol, 2 eq) at 0° C. The mixture was stirred at 40° C. for 1 hr. The mixture was diluted with water (10 mL), extracted with EA (15 mL*3) and washed with brine (5 mL*3). The organic layer was dried with anhydrous Na₂SO₄, filtered and concentrated under vacuum. The residue was purified by column chromatography (SiO₂, Petroleum ether/Ethyl acetate=1/0 to 0/1) and concentrated under vacuum to afford Intermediate 1-3 (4.7 g, 17.78 mmol, 66.59% yield) as a yellow oil. LCMS (Method E): Rt=0.537 min, [M+H]⁺=265.1. SFC: Rt=1.888 min, ee value >99%. ¹H NMR (400 MHz, DMSO-d6) δ=8.42-8.35 (m, 2H), 7.00-6.93 (m, 2H), 5.12 (br s, 1H), 3.63-3.54 (m, 1H), 3.47-3.36 (m, 2H), 3.31 (br s, 1H), 2.25-2.09 (m, 1H), 2.08-1.97 (m, 1H), 1.39 (br d, J=5.6 Hz, 9H).

### Step 2: Synthesis of Intermediate 1-4

A solution of Intermediate 1-3 (1 eq, 4 g) in IPA (100 mL) was prepared. A fixed bed (volume: 5 mL) was packed with granular catalyst (10% Ru/SiO₂, 2 g). The H₂ back pressure regulator was adjusted to 2.5 MPa and the flow rate of H₂ was 20 mL/min. Then the solution was pumped (0.303 mL/min) to the fixed bed (6.350(¼″) mm, 1 mL, 100° C.). Then the reaction mixture was collected from the reactor output. After the reaction fluid injection completed, the fixed bed was washed by extra IPA (100 mL). The organic layer was concentrated under vacuum. Without purification. Intermediate 1-4 (3.6 g, crude) as colorless oil was obtained. LCMS (Method E): Rt=0.372 min, [M+H]⁺=271.1. ¹H NMR (400 MHz, CHLOROFORM-d) δ=4.13 (br s, 1H), 3.72 (br s, 2H), 3.42-3.21 (m, 4H), 3.16-3.05 (m, 2H), 2.78-2.67 (m, 2H), 1.90 (br s, 4H), 1.60-1.47 (m, 2H), 1.43 (s, 9H).

### Step 3: Synthesis of Intermediate 1-6

To a solution of Intermediate 1-4 (500 mg, 1.85 mmol, 1 eq) in DMF (5 mL) was added Intermediate 1-5 (819.02 mg, 1.85 mmol, 1 eq), DIEA (956.05 mg, 7.40 mmol, 1.29 mL, 4 eq) and KI (1.23 g, 7.40 mmol, 4 eq). The mixture was stirred at 25° C. for 1 hr. The mixture was diluted with water (2 mL), extracted with EtOAc (10 mL*3) and washed with brine (5 mL*4). The organic layer was dried with anhydrous Na₂SO₄, filtered and concentrated under vacuum. The residue was purified by column chromatography (SiO₂, Dichloromethane: Methanol=1/0 to 10/1) and concentrated under vacuum to afford Intermediate 1-6 (700 mg, 1.03 mmol, 55.93% yield) as a white solid. LCMS (Method G): Rt=0.589 min, [M+H]⁺=677.3. SFC: Rt=2.048 min, ee value >99%. ¹H NMR (400 MHz, DMSO-d6) δ=12.59 (s, 1H), 8.26 (d, J=7.6 Hz, 1H), 8.00-7.94 (m, 1H), 7.88 (t, J=7.2 Hz, 1H), 7.85-7.79 (m, 1H), 7.43 (br s, 1H), 7.38 (br t, J=5.1 Hz, 1H), 7.23 (t, J=9.0 Hz, 1H), 4.33 (s, 2H), 4.14 (br s, 1H), 3.67-3.54 (m, 3H), 3.48 (br d, J=14.5 Hz, 2H), 3.39-3.29 (m, 5H), 3.24 (br s, 2H), 3.15 (br d, J=9.0 Hz, 4H), 3.07 (br s, 1H), 2.65 (br s, 2H), 2.17-2.04 (m, 2H), 1.79 (br d, J=12.6 Hz, 4H), 1.38 (br s, 9H). ¹⁹F NMR (377 MHz, DMSO-d6) δ=−73.41 (s, 1F), −119.76 (br s, 1F).

### Step 4: Synthesis of Intermediate 1-7

To a solution of Intermediate 1-6 (1 g, 1.48 mmol, 1 eq) in DCM (3 mL) was added HCl/dioxane (2 M, 6.00 mL). The mixture was stirred at 25° C. for 1 hr. The mixture was concentrated under vacuum. The residue was used in the next step without purification. Intermediate 1-7 (900 mg, crude, HCl salt) as a white solid was obtained. LCMS (Method G): Rt=0.655 min, [M+H]⁺=577.4.

### Step 5: Synthesis of I-875

To a solution of Intermediate 1-7 (990 mg, 1.61 mmol, 1 eq, HCl salt) in DMF (10 mL) was added Intermediate 1-8 (415.50 mg, 1.61 mmol, 1 eq), EDCI (619.08 mg, 3.23 mmol, 2 eq), HOAt (219.78 mg, 1.61 mmol, 225.88 μL, 1 eq) and NMM (816.60 mg, 8.07 mmol, 887.61 μL, 5 eq). The mixture was stirred at 25° C. for 1 hr. The mixture was diluted with water (10 mL), extracted with EtOAc (20 mL*3) and washed with brine (5 mL*3). The organic layer was dried with anhydrous Na₂SO₄, filtered and concentrated under vacuum to give a crude product. The crude product was triturated with PE/EA=20/1 (50 mL), then filtered and the filter cake was washed with PE/EA=20/1 (5 mL*3), the filter cake was dried under vacuum to give product (−1.1 g). 300 mg of the product was further purified by Prep-HPLC (column: Waters Xbridge 150*25 mm*5 um; mobile phase: [water (NH₄HCO₃)-ACN]; gradient: 30%-60% B over 15 min) and Prep-HPLC (column: Phenomenex luna C18 150*25 mm*10 um; mobile phase: [water (FA)-ACN]; gradient: 18%-48% B over 15 min), and the eluent was lyophilized to give I-875 (200 mg, 231.83 mol, 14.36% yield, 99.92% purity, FA salt) as a white solid. LCMS (Method G): Rt=0.630 min, [M+H]⁺=816.5. SFC: Rt=1.441 min, ee value >99%. ¹H NMR (400 MHz, DMSO-d6) δ=12.61 (s, 1H), 8.27 (d, J=7.6 Hz, 1H), 8.21 (s, 1H), 8.01-7.95 (m, 1H), 7.87-7.92 (m, 1H), 7.87-7.81 (m, 1H), 7.45 (br d, J=0.8 Hz, 1H), 7.35-7.41 (m, 1H), 7.21-7.27 (m, 1H), 6.95-6.43 (m, 1H), 4.34 (s, 2H), 4.26-4.14 (m, 1H), 4.03-3.83 (m, 1H), 3.73-3.37 (m, 10H), 3.30-3.07 (m, 5H), 2.70-2.58 (m, 2H), 2.19-2.06 (m, 2H), 1.95 (br s, 1H), 1.89-1.70 (m, 4H), 1.70-1.54 (m, 4H), 1.53-1.41 (m, 2H), 1.38-1.33 (m, 9H), 1.14-1.04 (m, 3H), 0.93 (br d, J=10.0 Hz, 2H). ¹⁹F NMR (377 MHz, DMSO-d6) δ=−119.80 (br s, 1F).

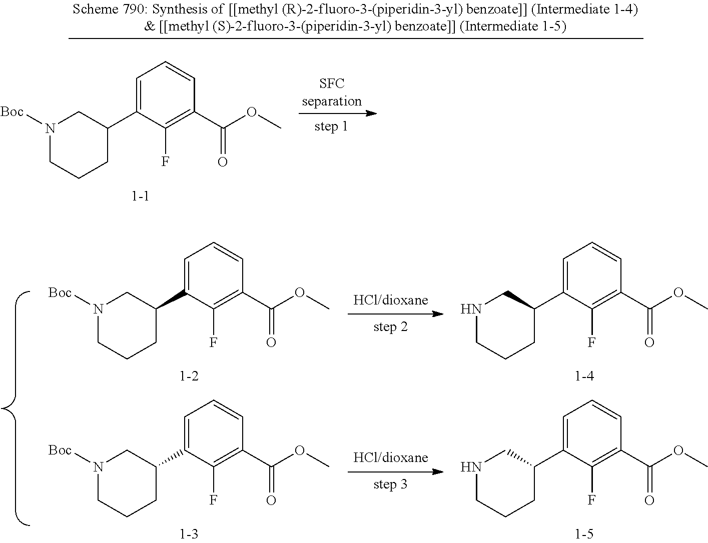 [SMILES: COC(=O)c1cccc(C2CCCN(C(=O)OC(C)(C)C)C2)c1F ; COC(=O)c1cccc([C@H]2CCCN(C(=O)OC(C)(C)C)C2)c1F ; COC(=O)c1cccc([C@H]2CCCNC2)c1F ; COC(=O)c1cccc([C@@H]2CCCN(C(=O)OC(C)(C)C)C2)c1F ; COC(=O)c1cccc([C@@H]2CCCNC2)c1F]

### Step 1: Synthesis of Intermediate 1-2 & Intermediate 1-3

Intermediate 1-1 (10 g, 29.64 mmol) was purified by SFC separation (column: DAICEL CHIRALPAK IG (250 mm*50 mm, 10 um); mobile phase: [CO₂-MeOH (0.1% NH₃H₂O)]; B %: 40%, isocratic elution mode). The eluent was concentrated under reduced pressure to afford products. Intermediate 1-2 (5 g, 14.09 mmol, 47.54% yield, 95.08% purity) as a yellow oil. Intermediate 1-3 (5 g, 14.33 mmol, 48.34% yield, 96.68% purity) was obtained as a yellow oil. LCMS: Retention time: 0.582 min, [M-Boc+H]⁺=238.1. SFC: Rt=0.864 min, ee value >99%. SFC: Rt=1.077 min, ee value=99.5%.

### Step 2: Synthesis of Intermediate 1-4

Intermediate 1-2 (4 g, 11.86 mmol, 1 eq) in DCM (20 mL) and HCl/dioxane (2 M, 20 mL) was stirred at 25° C. for 1 hr. The reaction was concentrated under reduced pressure to give a residue. It was used in the next step without further purification. Intermediate 1-4 (4.0 g, HCl salt) was obtained as a white solid. LCMS (Method E): Retention time: 0.675 min, [M+H]⁺=238.1. SFC: Rt=1.837 min, ee value=97.9%. ¹H NMR (400 MHz, METHANOL-d4) δ=7.89-7.80 (m, 1H), 7.62 (t, J=7.2 Hz, 1H), 7.30 (t, J=8.0 Hz, 1H), 3.91 (s, 3H), 3.53-3.37 (m, 3H), 3.22-3.12 (m, 1H), 3.11-3.01 (m, 1H), 2.15-1.85 (m, 4H). ¹⁹F NMR (377 MHz, METHANOL-d4) δ=−117.93-−118.53 (m, 1F).

### Step 3: Synthesis of Intermediate 1=5

Intermediate 1-3 (4.0 g, 11.86 mmol, 1 eq) in DCM (30 mL) and HCl/dioxane (2 M, 20 mL) was stirred at 25° C. for 1 hr. The reaction was concentrated under reduced pressure to give a residue. It was used in the next step without further purification. The product was combined with another lot of material. Intermediate 1-5 (4.0 g, HCl salt) was obtained as a white solid. LCMS (Method E): Retention time: 0.688 min, [M+H]⁺=238.1. SFC: Rt=0.549 min, ee value=99.3%. ¹H NMR (400 MHz, METHANOL-d4) δ=7.85 (t, J=7.2 Hz, 1H), 7.63 (t, J=7.2 Hz, 1H), 7.30 (t, J=8.0 Hz, 1H), 3.91 (s, 3H), 3.58-3.36 (m, 3H), 3.23-2.89 (m, 2H), 2.22-1.85 (m, 4H). ¹⁹F NMR (377 MHz, METHANOL-d4) δ=−118.28 (s, 1F).

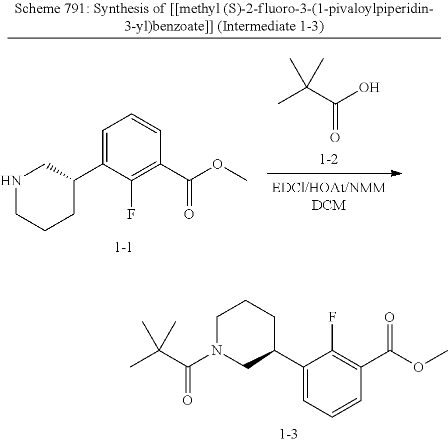

To a solution of Intermediate 1-1 (1.2 g, 4.38 mmol, 1 eq, HCl salt) and Intermediate 1-2 (447.73 mg, 4.38 mmol, 503.63 μL, 1 eq) in DCM (10 mL) was added EDCI (2.52 g, 13.15 mmol, 3 eq), HOAt (716.03 mg, 5.26 mmol, 735.90 μL, 1.2 eq) and NMM (2.22 g, 21.92 mmol, 2.41 mL, 5 eq). The mixture was stirred at 25° C. for 1 hr. The reaction was cooled to 20° C., which was diluted with H₂O (5 mL), extracted with DCM (5 mL*3), and the organic phase was washed with saturated aqueous NaCl (5 mL), then dried over Na₂SO₄, filtered and concentrated under reduced pressure to give a residue. The residue was purified by flash silica gel chromatography (ISCO; 40 g SepaFlash Silica Flash Column, Eluent of 0˜100% Ethyl acetate/Petroleum ether gradient @60 mL/min). The eluent was concentrated to afford intermediate 1-3 (1.3 g, 4.04 mmol, 92.27% yield, 100% purity) as a white solid. LCMS (Method E): Retention time: 0.976 min, [M+H]⁺=322.2. SFC: Rt=1.966 min, ee value >99%. ¹H NMR (400 MHz, CHLOROFORM-d) δ=7.86-7.79 (m, 1H), 7.47-7.40 (m, 1H), 7.20 (t, J=7.6 Hz, 1H), 4.65-4.42 (m, 2H), 3.95 (s, 3H), 3.15-3.02 (m, 1H), 3.01-2.88 (m, 1H), 2.87-2.72 (m, 1H), 2.10-2.00 (m, 1H), 1.90-1.74 (m, 2H), 1.72-1.62 (m, 1H), 1.32 (s, 9H). ¹⁹F NMR (377 MHz, CHLOROFORM-d) δ=−115.27 (br s, 1F).

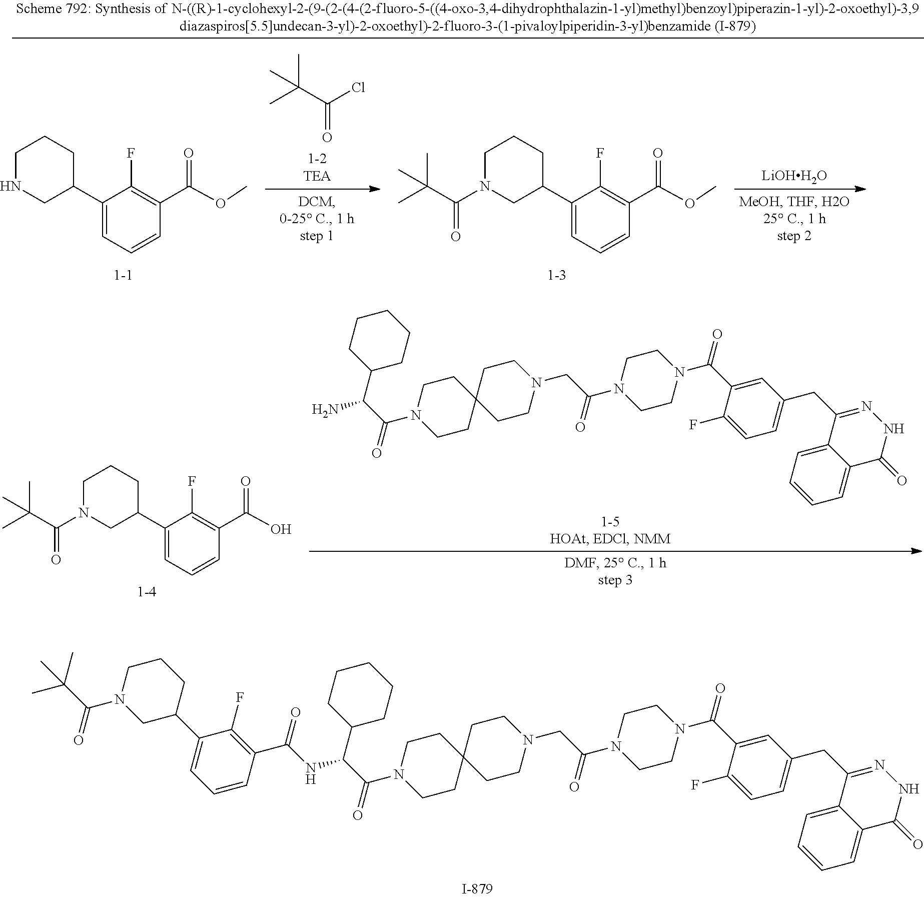

### Step 1: Synthesis of Intermediate 1-3

To a solution of Intermediate 1-1 (1 g, 3.65 mmol, 1 eq, HCl salt) and TEA (1.11 g, 10.96 mmol, 1.53 mL, 3 eq) in DCM (10 mL) was added dropwise Intermediate 1-2 (440.50 mg, 3.65 mmol, 449.49 L, 1 eq) at 0° C. The mixture was stirred at 25° C. for 1 hr. The mixture was poured into 20 mL of water and extracted with DCM (20 mL*2). The combined organic phase was dried over anhydrous Na₂SO₄, filtered and concentrated in vacuum to give a residue. The residue was purified by flash silica gel chromatography (ISCO; 40 g SepaFlash Silica Flash Column, Eluent of 0˜30% Ethyl acetate/Petroleum ether gradient @40 mL/min). The eluent was concentrated to give Intermediate 1-3 (1.05 g, 3.27 mmol, 89.43% yield) as a white solid. LCMS (Method E): Rt=0.626 min, [M+H]⁺=322.1. ¹H NMR (400 MHz, CHLOROFORM-d) δ=7.85-7.78 (m, 1H), 7.46-7.38 (m, 1H), 7.18 (t, J=7.7 Hz, 1H), 4.62-4.38 (m, 2H), 3.94 (s, 3H), 3.15-3.01 (m, 1H), 2.98-2.87 (m, 1H), 2.78 (br t, J=12.4 Hz, 1H), 2.02 (br s, 1H), 1.89-1.73 (m, 2H), 1.70-1.61 (m, 1H), 1.30 (s, 9H).

### Step 2: Synthesis of Intermediate 1-4

To a solution of Intermediate 1-3 (1.05 g, 3.27 mmol, 1 eq) in THF (10 mL), MeOH (10 mL) and H₂O (2 mL) was added LiOH·H₂O (274.20 mg, 6.53 mmol, 2 eq). The mixture was stirred at 25° C. for 2 hr. The mixture was concentrated in vacuum and the mixture was acidified to pH=2 with 1N aq. HCl, extracted with DCM (10 mL*2), the combined organic phase was dried over anhydrous Na₂SO₄, filtered and concentrated in vacuum to give Intermediate 1-4 (770 mg, 2.51 mmol, 76.68% yield) as a white solid. LCMS (Method E): Rt=0.548 min, [M+H]⁺=308.1. ¹H NMR (400 MHz, DMSO-d6) δ=7.94-7.87 (m, 1H), 7.48 (t, J=6.4 Hz, 1H), 7.22 (t, J=7.8 Hz, 1H), 4.54 (br t, J=12.9 Hz, 2H), 3.18-3.04 (m, 1H), 2.99-2.88 (m, 1H), 2.87-2.74 (m, 1H), 2.09-2.03 (m, 1H), 1.90-1.83 (m, 1H), 1.82-1.75 (m, 1H), 1.71-1.62 (m, 1H), 1.32 (s, 9H).

### Step 3: Synthesis of I-879

To a solution of Intermediate 1-4 (208.71 mg, 679.06 μmol, 1 eq) in DMF (10 mL) was added HOAt (184.85 mg, 1.36 mmol, 189.98 μL, 2 eq) and EDCI (260.35 mg, 1.36 mmol, 2 eq). The mixture was stirred at 25° C. for 30 min. Then Intermediate 1-5 (500 mg, 679.06 μmol, 1 eq, HCl salt) and NMM (274.74 mg, 2.72 mmol, 298.63 μL, 4 eq) were added and the mixture was stirred at 25° C. for 1 hr. The reaction was purified directly without further purification. The crude product was purified by reversed-phase HPLC (0.1% FA condition). The eluent was lyophilized to give I-879 (301.97 mg, 290.97 mol, 42.85% yield, 99.75% purity, FA salt) as a white solid. LCMS (Method E): Rt=0.528 min, [M+H]⁺=989.7. SFC: Rt=0.2.269 min, 3.197 min. ¹H NMR (400 MHz, CHLOROFORM-d) δ=10.72-10.30 (m, 1H), 8.53-8.41 (m, 1H), 8.18 (s, 1H), 7.87 (q, J=7.5 Hz, 1H), 7.83-7.67 (m, 3H), 7.54-7.41 (m, 1H), 7.39-7.30 (m, 3H), 7.24-7.17 (m, 1H), 7.06 (br t, J=8.8 Hz, 1H), 5.10 (br d, J=1.1 Hz, 1H), 4.59-4.42 (m, 2H), 4.30 (s, 2H), 3.85-3.46 (m, 10H), 3.40-3.23 (m, 4H), 3.11-3.03 (m, 1H), 3.00-2.74 (m, 6H), 2.61 (br s, 4H), 2.04 (br d, J=11.6 Hz, 1H), 1.86-1.71 (m, 7H), 1.69-1.60 (m, 4H), 1.49 (br s, 2H), 1.30 (s, 9H), 1.27-1.05 (m, 5H). ¹⁹F NMR (377 MHz, METHANOL-d₄) δ=−117.56 (br d, J=31.4 Hz, 1F), −118.73 (br d, J=38.1 Hz, 1F).

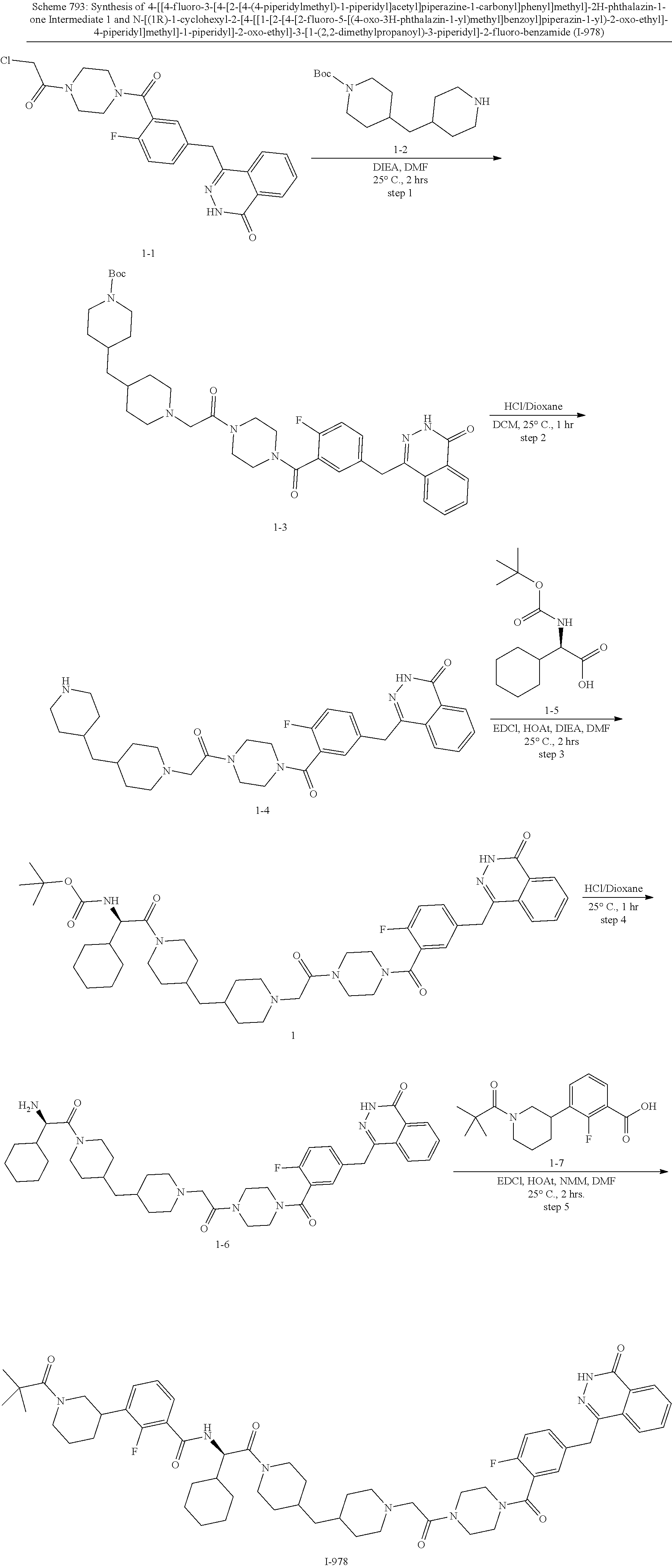

### Step 1: Synthesis of Intermediate 1-3

To a solution of intermediate 1-2 (382.62 mg, 1.35 mmol, 1 eq) in DMF (6 mL) was added intermediate 1-1 (600 mg, 1.35 mmol, 1 eq) and DIEA (262.64 mg, 2.03 mmol, 353.96 μL, 1.5 eq) and the reaction mixture was stirred at 25° C. for 2 hrs. The reaction mixture was worked up with another lot of material. The reaction mixture was quenched by water (10 mL), and extracted by DCM (10 mL*3), and washed by brine (10 mL*2). The organic phase was combined and concentrated to afford the crude. The crude product was purified by reverse phase chromatography (80 g of XB—C18, 20-40 μm, 120 Å) Mobile phase: A for H₂O (0.1% FAv/v) and B for acetonitrile; Gradient: B5%-100% in 30 min; Flow rate: 100 ml/min; Column temperature: R.T. Wavelength: 220 nm/254 nm). The eluent was concentrated to remove organic solvents and the pH was adjusted to 7 by with a saturated solution of NaHCO₃ (30 mL), and extracted by DCM (50 mL*3). The organic phase was combined, dried by anhydrous Na₂SO₄, and concentrated to give the product. intermediate 1-3 (600 mg, 810.07 μmol, 59.79% yield, 93% purity) was obtained as a yellow solid. LCMS (Method E): Rt=0.472 min, [M+H]⁺=689.5. ¹H NMR (400 MHz, CHLOROFORM-d) δ=10.51 (d, J=8.8 Hz, 1H), 8.54-8.40 (m, 1H), 7.89-7.64 (m, 3H), 7.41-7.30 (m, 2H), 7.05 (t, J=8.4 Hz, 1H), 4.29 (s, 2H), 4.06 (d, J=1.2 Hz, 2H), 3.87-3.53 (m, 6H), 3.40-3.22 (m, 2H), 3.21-3.10 (m, 2H), 2.92-2.75 (m, 2H), 2.74-2.59 (m, 2H), 2.12-1.96 (m, 3H), 1.78-1.62 (m, 5H), 1.46 (s, 9H), 1.22-1.03 (m, 6H).

### Step 2: Synthesis of Intermediate 1-4

A solution of intermediate 1-3 (500 mg, 725.87 μmol, 1 eq) in DCM (5 mL) was added HCl/dioxane (4 M, 5 mL, 27.55 eq). The reaction suspension was stirred at 25° C. for 1 hr. The reaction suspension was concentrated to give a residue. The mixture was purified by reversed phase HPLC (0.1% FA, 15%), and the major peak was collected and lyophilized to give the desired product, which was diluted with aq·Na₂CO₃ (10 mL) and extracted with DCM (20*3 mL). The combined organic layers were dried over Na₂SO₄, filtered and concentrated under reduced pressure to give a residue. Intermediate 1-4 (320 mg, 543.56 μmol, 74.88% yield) was obtained as a white solid. LCMS (Method E): Rt=0.367 min, [M+H]⁺=561.3.

### Step 3: Synthesis of Intermediate 1

To a mixture of Intermediate 1-5 in DMF (6 mL) was added EDCI (283.29 mg, 1.48 mmol, 3 eq), HOAt (100.57 mg, 738.90 μmol, 103.36 μL, 1.5 eq) and NMM (249.13 mg, 2.46 mmol, 270.80 μL, 5 eq). Then Intermediate 1-4 (290 mg, 492.60 μmol, 1 eq) was added. The reaction solution was stirred at 25° C. for 2 hr. The reaction solution was diluted with water (5 mL) and extracted with EA (5 mL*3). The combined organic layers were washed with brine (5 mL), dried over Na₂SO₄, filtered and concentrated under reduced pressure to give a residue. The residue was purified by prep-HPLC (0.1% FA condition), and the major peak was collected and concentrated to remove MeCN, then lyophilized to give the desired product. Intermediate 1 (230 mg, 263.14 μmol, 53.42% yield, 100% purity, FA salt) was obtained as a white solid. LCMS (Method E): Rt=0.502 min, (M+H)=828.6. SFC: Rt=1.512 min. ¹H NMR (400 MHz, DMSO-d₆) δ=12.60 (s, 1H), 8.26 (d, J=8.0 Hz, 1H), 8.17 (s, 1H), 8.00-7.94 (m, 1H), 7.89 (t, J=7.2 Hz, 1H), 7.86-7.79 (m, 1H), 7.48-7.41 (m, 1H), 7.37 (t, J=6.8 Hz, 1H), 7.24 (t, J=8.8 Hz, 1H), 6.75-6.61 (m, 1H), 4.43-4.28 (m, 3H), 4.21 (d, J=1.2 Hz, 1H), 4.01-3.97 (m, 1H), 3.70-3.54 (m, 3H), 3.49 (d, J=12.0 Hz, 2H), 3.40-3.34 (m, 2H), 3.21-3.10 (m, 3H), 3.07 (s, 1H), 2.97 (t, J=12.4 Hz, 1H), 2.85-2.69 (m, 2H), 2.02-1.86 (m, 2H), 1.60 (d, J=8.4 Hz, 11H), 1.35 (s, 9H), 1.31-1.21 (m, 1H), 1.20-0.83 (m, 11H). ¹⁹F NMR (377 MHz, DMSO-d₆) δ=−119.78 (br d, J=11.2 Hz, 1F).

### Step 4: Synthesis of Intermediate 1-6

To a solution of Intermediate 1 (200 mg, 228.82 μmol, 1 eq, FA salt) in DCM (2 mL) was added HCl/dioxane (4 M, 2 mL, 34.96 eq). The reaction suspension was stirred at 25° C. for 1 hr. The reaction suspension was concentrated to give a residue. The crude product Intermediate 1-6 (170 mg, 222.41 μmol, 97.20% yield, HCl salt) as a white solid was used in the next step without further purification. LCMS (Method E): Rt=0.413 min, (M+H)=728.5.

### Step 5: Synthesis of I-978

To a mixture of Intermediate 1-7 (60.32 mg, 196.24 μmol, 1 eq) in DMF (1.5 mL) was added EDCI (112.86 mg, 588.72 μmol, 3 eq), HOAt (40.07 mg, 294.36 μmol, 41.18 μL, 1.5 eq) and NMM (198.49 mg, 1.96 mmol, 215.75 μL, 10 eq). Then Intermediate 1-6 (150 mg, 196.24 μmol, 1 eq, HCl salt) was added. The reaction solution was stirred at 25° C. for 2 hrs. The residue was diluted with water (3 mL) and extracted with EA (3 mL*3). The combined organic layers were washed with brine (3 mL), dried over Na₂SO₄, filtered and concentrated under reduced pressure to give a residue. The residue was purified by reversed phase chromatography (ISCO; 80 g SepaFlash C18 Column, Eluent of 5˜40%˜95% (0.1% FA) water/MeCN@40 mL/min), and the major peak was collected and lyophilized to give the desired product. I-978 (180.72 mg, 177.66 μmol, 90.53% yield, 100% purity) was obtained as a white solid. LCMS (Method E): Rt=0.536 min, (M+H)=1017.6. SFC: Rt=2.031, 2.780 min. ¹H NMR (400 MHz, DMSO-d₆) δ=12.61 (s, 1H), 8.44-8.32 (m, 1H), 8.27 (d, J=7.6 Hz, 1H), 8.02-7.95 (m, 1H), 7.90 (t, J=7.5 Hz, 1H), 7.86-7.79 (m, 1H), 7.55-7.34 (m, 4H), 7.31-7.16 (m, 2H), 4.79 (t, J=8.0 Hz, 1H), 4.48-4.24 (m, 5H), 4.20-3.97 (m, 1H), 3.74-3.55 (m, 3H), 3.54-3.45 (m, 2H), 3.31-3.23 (m, 2H), 3.21-3.11 (m, 3H), 3.10-2.99 (m, 2H), 2.97-2.71 (m, 5H), 2.63-2.56 (m, 1H), 2.01-1.86 (m, 3H), 1.81-1.55 (m, 12H), 1.53-1.42 (m, 1H), 1.35-1.27 (m, 1H), 1.21 (s, 9H), 1.19-0.94 (m, 10H), 0.93-0.79 (m, 1H). ¹⁹F NMR (377 MHz, DMSO-d₆) δ=−119.78 (d, J=9.0 Hz, 1F), −120.00 (s, 1F), −120.30 (d, J=24.7 Hz, 1F).

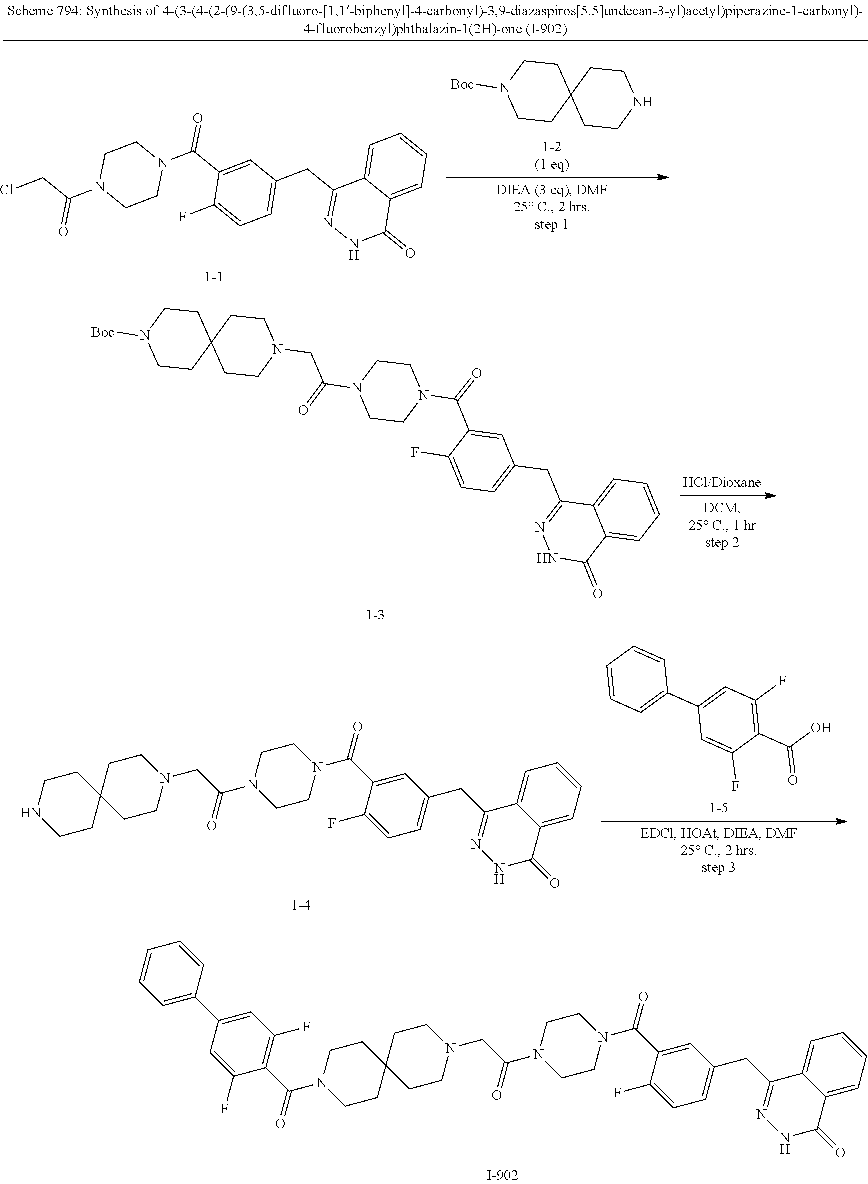

### Step 1: Synthesis of Intermediate 1-3

To a solution of intermediate 1-2 (287.18 mg, 1.13 mmol, 1 eq) in DMF (5 mL) was added intermediate 1-1 (500 mg, 1.13 mmol, 1 eq) and DIEA (218.87 mg, 1.69 mmol, 294.97 μL, 1.5 eq) and the reaction mixture was stirred at 25° C. for 2 hrs. The reaction mixture was quenched by water (10 mL), and extracted by DCM (10 mL*3), and washed by brine (10 mL*2). The organic phase was combined and concentrated to afford the crude. The crude product was purified by reverse phase chromatography (80 g of XB—C18, 20-40 μm, 120 Å) Mobile phase: A for H₂O (0.1% FAv/v) and B for acetonitrile; Gradient: B5%-100% in 30 min; Flow rate: 100 ml/min; Column temperature: R.T. Wavelength: 220 nm/254 nm), then the eluent was concentrated to remove organic solvents and the pH was adjusted to 7 by a saturated solution of NaHCO₃ (30 mL), and extracted by DCM (50 mL*3). The organic phase was combined, dried by anhydrous Na₂SO₄, and concentrated to afford Intermediate 1-3 (500 mg, 726.42 μmol, 64.34% yield, 96% purity) as a yellow solid. LCMS (Method D): Rt=0.425 min, [M+H]⁺=661.4. ¹H NMR (400 MHz, CHLOROFORM-d) δ=10.64-10.22 (m, 1H), 8.60-8.34 (m, 1H), 7.86-7.63 (m, 3H), 7.42-7.29 (m, 2H), 7.05 (t, J=8.4 Hz, 1H), 4.29 (s, 2H), 3.88-3.66 (m, 4H), 3.65-3.49 (m, 2H), 3.36 (d, J=4.0 Hz, 4H), 3.27 (br s, 1H), 3.23-3.14 (m, 2H), 2.46 (br d, J=4.8 Hz, 3H), 1.71 (br s, 3H), 1.57-1.36 (m, 16H).

### Step 2: Synthesis of Intermediate 1-4

A solution of Intermediate 1-3 (400 mg, 605.35 μmol, 1 eq) in DCM (4 mL) was added HCl/dioxane (4 M, 4 mL, 26.43 eq). The reaction suspension was stirred at 25° C. for 1 hr. The reaction suspension was concentrated to give the residue. The mixture was purified by reversed phase HPLC (0.1% FA, 15%), and the eluent was collected and lyophilized to give the desired product, which was diluted with a sat.aq·Na₂CO₃ solution (10 mL) and extracted with DCM (20*3 mL). The combined organic layers were dried over Na₂SO₄, filtered and concentrated under reduced pressure to give Intermediate 1-4 (250 mg, 445.90 μmol, 73.66% yield) as a white solid. LCMS (Method E): Rt=0.354 min, [M+H]⁺=561.2.

### Step 3: Synthesis of I-902

To a mixture of Intermediate 1-5 (105.68 mg, 451.25 μmol, 1.1 eq) in DMF (5 mL) was added EDCI (235.92 mg, 1.23 mmol, 3 eq), HOAt (83.76 mg, 615.35 μmol, 86.08 μL, 1.5 eq) and NMM (207.47 mg, 2.05 mmol, 225.51 μL, 5 eq). Then Intermediate 1-4 (230 mg, 410.23 μmol, 1 eq) was added. The reaction solution was stirred at 25° C. for 2 hrs. The reaction solution was diluted with water (5 mL) and extracted with EA (5 mL*3). The combined organic layers were washed with brine (5 mL), dried over Na₂SO₄, filtered and concentrated under reduced pressure to give a residue. The residue was purified by prep-HPLC (0.1% FA condition), the eluent was collected and concentrated to remove MeCN, then lyophilized to give I-902 (195.92 mg, 237.82 μmol, 57.97% yield, 99.884% purity, FA salt) as a white solid. LCMS (Method E): Rt=0.473 min, [M+H]⁺=777.4. ¹H NMR (400 MHz, DMSO-d₆) δ=12.60 (s, 1H), 8.27-8.25 (m, 1H), 8.16 (s, 1H), 8.00-7.94 (m, 1H), 7.89 (t, J=7.6 Hz, 1H), 7.82 (d, J=4.0 Hz, 1H), 7.80-7.75 (m, 2H), 7.58 (d, J=9.2 Hz, 2H), 7.53-7.41 (m, 4H), 7.40-7.33 (m, 1H), 7.23 (t, J=8.8 Hz, 1H), 4.33 (s, 2H), 3.69-3.55 (m, 5H), 3.52-3.44 (m, 2H), 3.35 (s, 1H), 3.26 (br s, 2H), 3.19-3.06 (m, 4H), 2.44-2.32 (m, 4H), 1.55-1.31 (m, 8H). ¹⁹F NMR (377 MHz, DMSO-d₆) δ=−113.83 (s, 2F), −119.77 (br d, J=13.5 Hz, 1F).

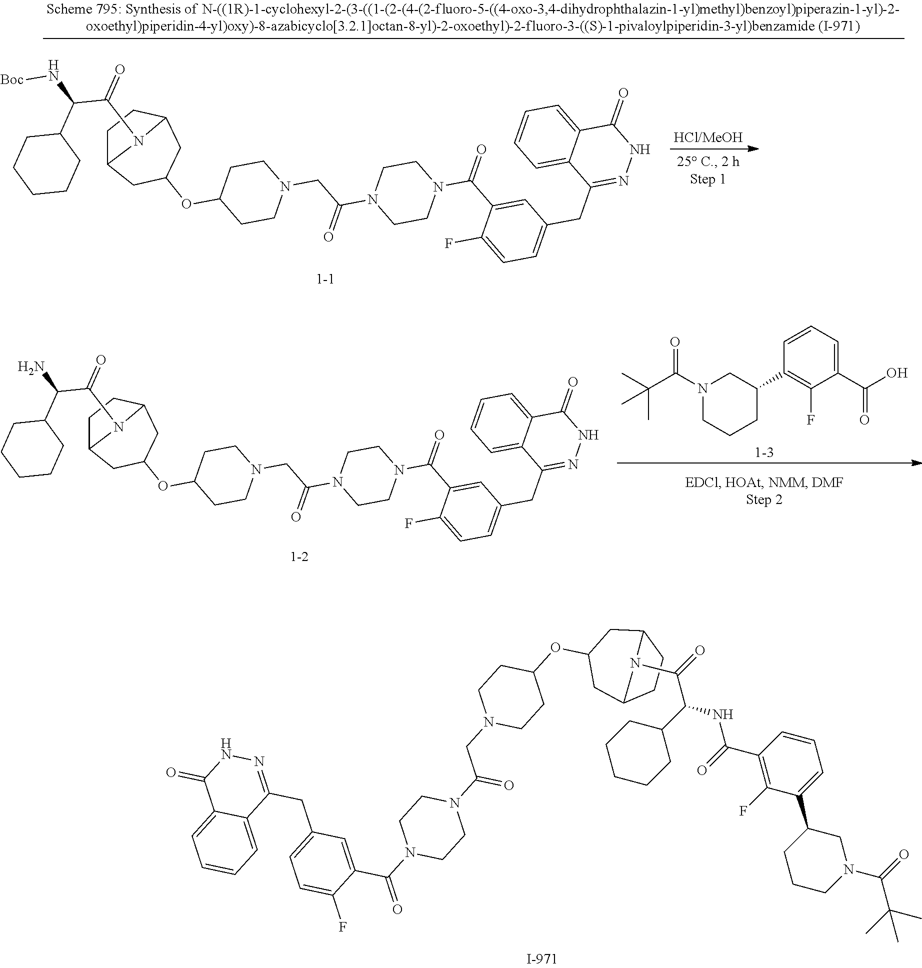

### Step 1: Synthesis of Intermediate 1-2

A solution of Intermediate 1-1 (0.8 g, 934.54 μmol, 1 eq) in HCl/MeOH (2 M, 16 mL) was stirred at 25° C. for 2 hr. The reaction mixture was concentrated in a vacuum to give Intermediate 1-2 (0.8 g, crude, HCl salt) as a white solid. The residue was used for the next step. LCMS (Method E): Rt=0.414 min, [M+H]⁺=756.5.

### Step 2: Synthesis of I-971

To a solution of Intermediate 1-2 (350 mg, 463.01 μmol, 1 eq) in DMF (7 mL) was added Intermediate 1-3 (142.31 mg, 463.01 μmol, 1 eq), EDCI (266.28 mg, 1.39 mmol, 3 eq), HOAt (63.02 mg, 463.01 μmol, 1 eq) and NMM (374.66 mg, 3.70 mmol, 407.24 μL, 8 eq), and the mixture was stirred at 20° C. for 1 hr. The mixture diluted with water (20 mL), and extracted with EA (20 mL*2). The combined organic layers were washed with brine (20 mL), dried over Na₂SO₄, filtered and concentrated under reduced pressure to give a residue. The residue was purified by prep-HPLC (column: Phenomenex luna C18 150*25 mm*10 um; mobile phase: [water (FA)-ACN]; gradient: 30%-60% B over 11 min), and concentrated to remove organic solvents. The residual aqueous solution was lyophilized to give I-971 (154 mg, 145.56 μmol, 31.44% yield, 98.8% purity) as a white solid. LCMS (Method E): Rt=0.547 min, [M+H]⁺=1045.6. SFC: Rt=1.137 min/2.568 min. ¹H NMR (400 MHz, METHANOL-d4) δ=8.46 (s, 1H), 8.37 (br d, J=7.6 Hz, 1H), 7.99-7.92 (m, 1H), 7.92-7.80 (m, 2H), 7.60-7.45 (m, 3H), 7.42-7.33 (m, 1H), 7.31-7.21 (m, 1H), 7.21-7.11 (m, 1H), 4.85-4.59 (m, 3H), 4.49 (br d, J=13.2 Hz, 3H), 4.39 (s, 2H), 3.88-3.42 (m, 10H), 3.40-3.33 (m, 2H), 3.12-2.87 (m, 5H), 2.72 (br dd, J=2.8, 6.0 Hz, 2H), 2.43-2.16 (m, 2H), 2.15-1.52 (m, 21H), 1.38-1.21 (m, 12H), 1.19-1.03 (m, 2H). ¹⁹F NMR (377 MHz, METHANOL-d4) δ=−120.72 (br d, J=22.4 Hz, 1F), −121.29-−121.82 (m, 1F).

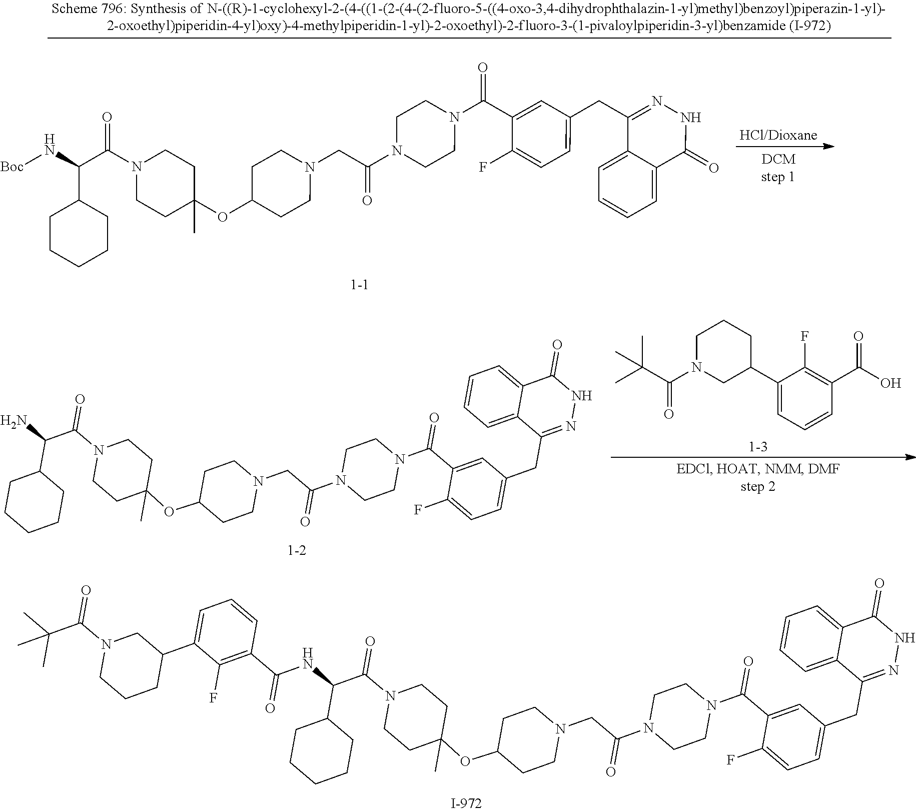

### Step 1: Synthesis of I-972

To a solution of Intermediate 1-1 (400 mg, 473.92 μmol, 1 eq) in DCM (6 mL) was added HCl/Dioxane (4 M, 2.00 mL, 16.88 eq). The mixture was stirred at 25° C. for 1 hr. The mixture was concentrated under vacuum to give a crude product. Intermediate 1-2 (360 mg, crude, HCl salt) was obtained as alight yellow solid which was used for the next step directly without further purification. LCMS (Method G): Rt=0.579 min, [M+H]⁺=744.5.

### Step 2: Synthesis of I-972

To a solution of Intermediate 1-3 (130 mg, 422.96 μmol, 1 eq), HOAt (57.57 mg, 422.96 μmol, 59.17 μL, 1 eq), EDCI (162.16 mg, 845.92 μmol, 2 eq), and NMM (213.91 mg, 2.11 mmol, 232.51 μL, 5 eq) in DMF (2 mL) was added Intermediate 1-2 (346.57 mg, 444.11 μmol, 1.05 eq, HCl salt). The mixture was stirred at 25° C. for 1 hr. The reaction mixture was diluted with water (20 mL) and extracted with EA (10 mL*3). The combined organic layers were washed with brine (10 mL), dried over Na₂SO₄, filtered and concentrated to give a residue. The residue was purified by Prep-HPLC (column: Waters Xbridge Prep OBD C18 150*40 mm*10 um; mobile phase: [water (NH₄HCO₃)-ACN]; gradient: 40%-70% B over 15 min), and then lyophilized to afford product. I-972 (387.98 mg, 366.93 μmol, 86.75% yield, 97.72% purity) was obtained as an off-white solid. LCMS (Method G): Rt=0.743 min, [M+H]⁺=1033.6. SFC: Rt=2.012 min, 2.811 min. ¹H NMR (400 MHz, METHANOL-d4) δ=8.41-8.33 (m, 1H), 7.99-7.79 (m, 3H), 7.60-7.44 (m, 3H), 7.42-7.34 (m, 1H), 7.29-7.21 (m, 1H), 7.20-7.12 (m, 1H), 4.98-4.87 (m, 2H), 4.85-4.81 (m, 1H), 4.59-4.46 (m, 2H), 4.39 (d, J=3.2 Hz, 2H), 4.24-3.87 (m, 2H), 3.86-3.71 (m, 3H), 3.70-3.49 (m, 5H), 3.36-3.33 (m, 1H), 3.27-3.23 (m, 1H), 3.22-3.16 (m, 1H), 3.12-3.04 (m, 1H), 3.02-2.88 (m, 2H), 2.87-2.71 (m, 2H). ¹⁹F NMR (376 MHz, METHANOL-d4) δ=−120.76 (br d, J=11.2 Hz, 1F), −121.34-−121.86 (m, 1F).

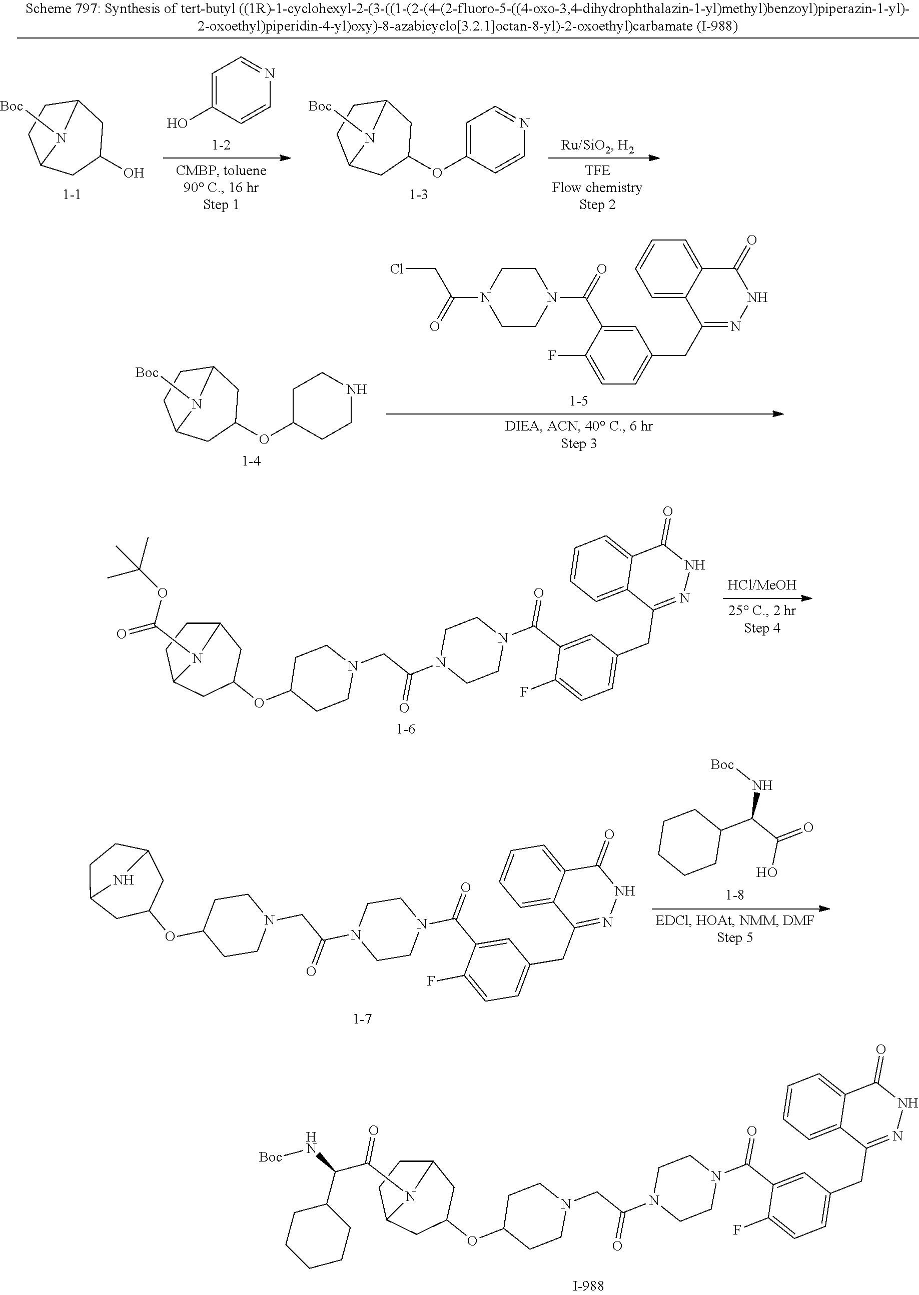

### Step 1: Synthesis of Intermediate 1-3

To a solution of Intermediate 1-1 (9 g, 39.60 mmol, 1 eq) in toluene (90 mL) was added Intermediate 1-2 (3.77 g, 39.60 mmol, 1 eq) and CMBP (14.33 g, 59.39 mmol, 1.5 eq), and the mixture was stirred at 90° C. for 16 hr. The mixture diluted with water (100 mL), and extracted with EA (100 mL*2). The combined organic layers were washed with brine (100 mL), dried over Na₂SO₄, filtered and concentrated under reduced pressure to give a residue. The crude product was purified by flash silica gel chromatography (PE:EA=0:1, Rf=0.2, ISCO; 330 g SepaFlash Silica Flash Column, Eluent of 0˜100% EA/PE, gradient @100 mL/min), and the eluent was concentrated under vacuum to give Intermediate 1-3 (8.2 g, 26.40 mmol, 66.68% yield, 98% purity) as a yellow oil. LCMS (Method E): Rt=0.428 min, [M+H]⁺=305.3. SFC: Rt=1.413 min/1.536 min. ¹H NMR (400 MHz, CHLOROFORM-d) δ=8.52-8.29 (m, 2H), 6.84-6.63 (m, 2H), 4.68 (t, J=4.8 Hz, 1H), 4.43-4.14 (m, 2H), 2.40-2.03 (m, 5H), 2.02-1.88 (m, 3H), 1.60-1.41 (m, 9H).

### Step 2: Synthesis of Intermediate 1-4

Intermediate 1-3 (7.7 g, 25.2 mmol, 1 eq) was dissolved in TFE (192.5 mL). A fixed bed (volume: 5 mL) was packed with granular catalyst (10% Ru/SiO₂, 3 g). The H₂ back pressure regulator was adjusted to 2.5 MPa, and the flow rate of H₂ was 20 mL/min. Then the solution was pumped (0.303 mL/min) to the fixed bed (6.350(¼″) mm, 1 mL, 100° C.). Then the reaction mixture was collected from the reactor output. The reaction mixture was concentrated under reduced pressure to give Intermediate 1-4 (7.5 g, 24.16 mmol, 97.40% yield) as a colorless oil. LCMS (Method E): Rt=0.424 min, [M+H]⁺=311.3. ¹H NMR (400 MHz, CHLOROFORM-d) δ=4.38-4.04 (m, 2H), 3.71 (t, J=4.8 Hz, 1H), 3.42 (dt, J=4.8, 8.4 Hz, 1H), 3.14-3.00 (m, 1H), 3.14-2.99 (m, 2H), 2.73-2.55 (m, 3H), 2.19-1.70 (m, 9H), 1.60 (br d, J=6.0 Hz, 1H), 1.52-1.34 (m, 11H).

### Step 3: Synthesis of Intermediate 1-6

To a solution of Intermediate 1-4 (2 g, 6.44 mmol, 1 eq) in ACN (20 mL) was added Intermediate 1-5 (2.85 g, 6.44 mmol, 1 eq) and DIEA (2.50 g, 19.33 mmol, 3.37 mL, 3 eq), and the mixture was stirred at 40° C. for 6 hr. The mixture was diluted with water (50 mL), and extracted with EA (50 mL*2). The combined organic layers were washed with brine (50 mL), dried over Na₂SO₄, filtered and concentrated under reduced pressure to give a residue. The crude product was purified by Prep-HPLC (column: Phenomenex luna C18 (250*70 mm, 10 um); mobile phase: [water (FA)-ACN]; gradient: 20%-500% B over 20 min) and the eluent was concentrated to remove ACN and lyophilized to give Intermediate 1-6 (2.5 g, 3.39 mmol, 52.56% yield, 97.1% purity) as a white solid. LCMS (Method E): Rt=0.480 min, [M+H]⁺=717.5. ¹H NMR (400 MHz, CHLOROFORM-d) δ=10.81-10.47 (m, 1H), 8.56-8.40 (m, 1H), 8.23 (s, 1H), 7.88-7.67 (m, 3H), 7.40-7.30 (m, 2H), 7.06 (t, J=8.8 Hz, 1H), 4.50-4.01 (m, 8H), 3.91-3.57 (m, 6H), 3.52-3.19 (m, 5H), 2.89 (br d, J=6.8 Hz, 2H), 2.72-2.42 (m, 2H), 2.17-1.93 (m, 4H), 1.75-1.56 (m, 4H), 1.46 (s, 9H).

### Step 4: Synthesis of Intermediate 1-7

A solution of Intermediate 1-6 (2.2 g, 3.07 mmol, 1 eq) in HCl/MeOH (2 M, 44 mL) was stirred at 25° C. for 2 hr. The reaction mixture was concentrated in a vacuum to give Intermediate 1-7 (2.2 g, HCl salt) as a white solid. The residue was used for the next step. LCMS (Method E): Rt=0.381 min, [M+H]⁺=603.4

### Step 4: Synthesis of I-988

To a solution of Intermediate 1-7 (2.2 g, 3.37 mmol, 1 eq, HCl salt) in DMF (22 mL) was added Intermediate 1-8 (866.70 mg, 3.37 mmol, 1 eq), EDCI (1.94 g, 10.10 mmol, 3 eq), HOAt (458.44 mg, 3.37 mmol, 1 eq) and NMM (2.73 g, 26.94 mmol, 2.96 mL, 8 eq), and the mixture was stirred at 25° C. for 4 hr. The mixture was diluted with water (50 mL), and extracted with EA (50 mL*2). The combined organic layers were washed with brine (50 mL), dried over Na₂SO₄, filtered and concentrated under reduced pressure to give a residue. The crude product was purified by Prep-HPLC (column: Phenomenex luna C18 (250*70 mm, 10 um); mobile phase: [water (FA)-ACN]; gradient: 28%-588% B over 18 min) and the eluent was concentrated to remove ACN. The aqueous phase was lyophilized to give I-988 (2.5 g, 2.86 mmol, 84.97% yield, 98% purity, FA salt) as a white solid. LCMS (Method E): Rt=0.509 min, [M+H]⁺=856.5. SFC: Rt=0.623 min/0.722 min. ¹H NMR (400 MHz, METHANOL-d4) δ=8.38 (m, 1H), 8.37-8.35 (m, 1H), 8.00-7.91 (m, 1H), 7.91-7.80 (m, 2H), 7.50 (m, 1H), 7.42-7.33 (m, 1H), 7.17 (m, 1H), 6.77-6.42 (m, 1H), 4.74-4.45 (m, 2H), 4.39 (s, 2H), 4.29-4.08 (m, 1H), 4.06-3.34 (m, 12H), 3.25-3.08 (m, 2H), 3.06-2.82 (m, 2H), 2.37-2.13 (m, 2H), 2.11-1.53 (m, 17H), 1.50-1.39 (m, 9H), 1.33-1.13 (m, 3H), 1.11-0.91 (m, 2H).

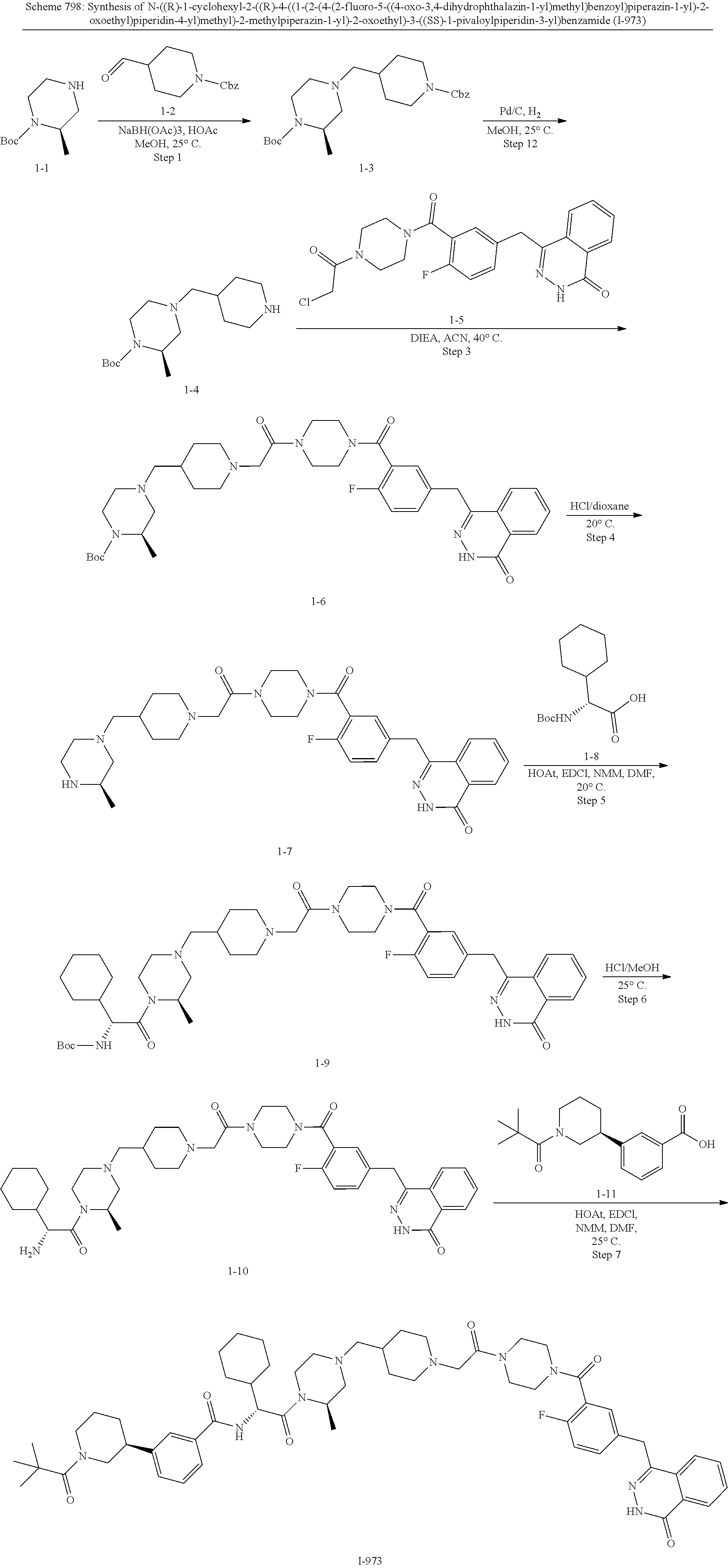

### Step 1: Synthesis of Intermediate 1-3

To a solution of Intermediate 1-1 (4 g, 19.97 mmol, 1 eq) and Intermediate 1-2 (4.94 g, 19.97 mmol, 1 eq) in MeOH (40 mL) was added and HOAc (1.20 g, 19.97 mmol, 1.14 mL, 1 eq) and the mixture was stirred for 0.1 hr. Then NaBH(OAc)₃ (8.47 g, 39.94 mmol, 2 eq) was added into the system. The mixture was stirred at 25° C. for 1 hr. The reaction mixture was diluted with H₂O (20 mL) and extracted with EA (20 mL*3). The combined organic layers were dried over Na₂SO₄, filtered and concentrated under reduced pressure to give a residue. The residue was purified by flash silica gel chromatography (ISCO; 80 g SepaFlash Silica Flash Column, Eluent of 0˜26% Ethyl acetate/Petroleum ethergradient @80 mL/min), and the eluent was concentrated under reduced pressure to give Intermediate 1-3 (8 g, 18.14 mmol, 90.81% yield, 97.838% purity) as a yellow oil. LCMS (Method D): Rt=0.287 min, [M+H]⁺=432.4. SFC: Rt=1.111 min. ¹H NMR (400 MHz, METHANOL-d4) δ=7.43-7.19 (m, 5H), 5.10 (s, 2H), 4.25-4.12 (m, 2H), 3.77 (d, J=13.2 Hz, 1H), 3.13-3.04 (m, 1H), 2.90-2.75 (m, 3H), 2.71 (d, J=11.2 Hz, 1H), 2.24-2.05 (m, 3H), 1.99-1.93 (m, 4H), 1.83-1.70 (m, 3H), 1.45 (s, 9H), 1.25-1.22 (m, 3H).

### Step 2: Synthesis of Intermediate 1-4

To a solution of Intermediate 1-3 (1 g, 2.32 mmol, 1 eq) in MeOH (10 mL) was added Pd/C (246.59 mg, 231.71 μmol, 0.1 eq) under N₂ atmosphere. The mixture was stirred under H₂ (15 Psi.) at 25° C. for 16 h. The reaction mixture was filtered and the filtrate was concentrated to give Intermediate 1-4 (677 mg, 2.28 mmol, 98.23% yield) as a white solid. ¹H NMR (400 MHz, CHLOROFORM-d) δ=4.19 (br s, 1H), 3.79 (br d, J=12.9 Hz, 1H), 3.30 (br s, 3H), 3.06 (dt, J=2.9, 12.6 Hz, 1H), 2.78-2.64 (m, 3H), 2.54 (br d, J=10.9 Hz, 1H), 2.22-2.07 (m, 3H), 1.97-1.85 (m, 3H), 1.72-1.61 (m, 1H), 1.46 (s, 9H), 1.41-1.30 (m, 2H), 1.21 (d, J=6.8 Hz, 3H).

### Step 3: Synthesis of Intermediate 1-6

To a solution of Intermediate 1-4 (0.65 g, 2.19 mmol, 1 eq) in ACN (6 mL) was added Intermediate 1-5 (967.83 mg, 2.19 mmol, 1 eq) and DIEA (847.32 mg, 6.56 mmol, 1.14 mL, 3 eq), and the mixture was stirred at 40° C. for 6 h. The mixture diluted with water (20 mL), and extracted with EA (20 mL*2). The combined organic layers were washed with brine (20 mL), dried over Na₂SO₄, filtered and concentrated under reduced pressure to give a residue. The crude product was purified by reversed-phase HPLC (0.1% FA condition), then concentrated to remove organic solvents. The residual aqueous solution was extracted with EA (20 mL*2). The combined organic layers were washed with brine (20 mL), dried over Na₂SO₄, filtered and concentrated under reduced pressure to give Intermediate 1-6 (1 g, 1.39 mmol, 63.71% yield, 98% purity) as a white solid. LCMS (Method E): Rt=0.411 min, [M+H]⁺=704.5. SFC: Rt=2.862 min. ¹H NMR (400 MHz, CHLOROFORM-d) δ=10.42-10.04 (m, 1H), 8.55-8.40 (m, 1H), 8.57-8.38 (m, 1H), 7.87-7.57 (m, 3H), 7.42-7.29 (m, 2H), 7.06 (brt, J=8.8 Hz, 1H), 4.29 (s, 2H), 4.23-4.14 (m, 1H), 3.78 (br d, J=14.0 Hz, 4H), 3.68-3.53 (m, 2H), 3.41-3.23 (m, 2H), 3.23-3.13 (m, 2H), 3.07 (br t, J=12.0 Hz, 1H), 2.93-2.76 (m, 2H), 2.69 (br d, J=7.2 Hz, 1H), 2.56 (br d, J=11.2 Hz, 1H), 2.20-2.06 (m, 4H), 1.93 (br t, J=10.4 Hz, 1H), 1.83-1.71 (m, 2H), 1.69-1.49 (m, 2H), 1.48-1.43 (m, 9H), 1.22 (br d, J=6.4 Hz, 5H).

### Step 4: Synthesis of Intermediate 1-7

A solution of Intermediate 1-6 (0.9 g, 1.28 mmol, 1 eq) in HCl/dioxane (2 M, 18 mL, 28.15 eq) was stirred at 20° C. for 12 h. The reaction mixture was concentrated in a vacuum to give Intermediate 1-7 (0.9 g, crude, HCl salt) as a white solid. The residue was used for the next step. LCMS (Method E): Rt=0.374 min, [M+H]⁺=604.5.

### Step 5: Synthesis of Intermediate 1-9

To a solution of Intermediate 1-7 (0.9 g, 1.41 mmol, 1 eq, HCl salt) in DMF (9 mL) was added Intermediate 1-8 (361.76 mg, 1.41 mmol, 1 eq), EDCI (808.50 mg, 4.22 mmol, 3 eq), HOAt (191.35 mg, 1.41 mmol, 196.66 μL, 1 eq) and NMM (1.14 g, 11.25 mmol, 1.24 mL, 8 eq), and the mixture was stirred at 20° C. for 1 h. The mixture diluted with water (20 mL), and extracted with EA (20 mL*2). The combined organic layers were washed with brine (20 mL), dried over Na₂SO₄, filtered and concentrated under reduced pressure to give a residue. The crude product was dissolved in MeOH (5 mL) and purified by Prep-HPLC (column: Phenomenex luna C18 150*40 mm*15 um; mobile phase: [water (FA)-ACN]; gradient: 10%-40% B over 22 min) and the eluent was concentrated to remove ACN. The aqueous phase was lyophilized to give Intermediate 1-9 (0.45 g, 533.78 μmol, 37.97% yield) as a white solid. LCMS (Method E): Rt=0.448 min, [M+H]⁺=843.5. SFC: Rt=0.948 min. ¹H NMR (400 MHz, CHLOROFORM-d) δ=8.51-8.37 (m, 1H), 7.84-7.69 (m, 3H), 7.32 (br d, J=5.6 Hz, 2H), 7.10-6.95 (m, 1H), 5.48-5.29 (m, 1H), 5.02-4.60 (m, 6H), 4.48-4.33 (m, 2H), 4.29 (s, 2H), 3.90-3.40 (m, 9H), 3.40-3.21 (m, 4H), 2.87-2.71 (m, 2H), 2.64 (br d, J=11.2 Hz, 2H), 2.28-1.93 (m, 4H), 1.86 (br s, 2H), 1.80-1.67 (m, 3H), 1.50-1.33 (m, 12H), 1.30-0.90 (m, 7H).

### Step 6: Synthesis of Intermediate 1-10

A solution of Intermediate 1-9 (0.4 g, 474.47 μmol, 1 eq) in HCl/MeOH (2 M, 8 mL) was stirred at 25° C. for 1 h. The reaction mixture was concentrated in a vacuum to give Intermediate 1-10 (0.4 g, crude, HCl salt) as a white solid. The residue was used for the next step. LCMS (Method E): Rt=0.411 min, [M+H]⁺=743.4.

### Step 7: Synthesis of I-973

To a solution of Intermediate 1-10 (0.4 g, 513.23 μmol, 1 eq, HCl salt) in DMF (5 mL) was added Intermediate 1-11 (148.51 mg, 513.23 μmol, 1 eq), EDCI (295.16 mg, 1.54 mmol, 3 eq), HOAt (69.86 mg, 513.23 μmol, 71.79 μL, 1 eq) and NMM (415.29 mg, 4.11 mmol, 451.40 μL, 8 eq), and the mixture was stirred at 25° C. for 1 h. The mixture diluted with water (20 mL), and extracted with EA (20 mL*2). The combined organic layers were washed with brine (20 mL), dried over Na₂SO₄, filtered and concentrated under reduced pressure to give a residue. The crude product was dissolved in DMF (5 mL) and purified by Prep-HPLC (column: Phenomenex luna C18 150*40 mm*15 um; mobile phase: [water (FA)-ACN]; gradient: 18%-48% B over 15 min) and the eluent was concentrated to remove ACN. The aqueous phase was lyophilized to give I-973 (308 mg, 299.20 μmol, 58.30% yield, 98.53% purity) as a white solid. LCMS (Method E): Rt=0.479 min, [M/2+H]⁺=508.1. SFC: Rt=1.694 min. ¹H NMR (400 MHz, METHANOL-d4) δ=8.37 (m, 1H), 7.99-7.92 (m, 1H), 7.91-7.81 (m, 2H), 7.75 (s, 1H), 7.70 (m, 1H), 7.54-7.32 (m, 4H), 7.17 (m, 1H), 4.74-4.60 (m, 1H), 4.48 (m, 2H), 4.39 (s, 2H), 4.09-4.00 (m, 2H), 3.95 (m, 1H), 3.86-3.67 (m, 3H), 3.61-3.52 (m, 2H), 3.49-3.33 (m, 5H), 3.10-2.69 (m, 8H), 2.34-1.41 (m, 21H), 1.37-1.21 (m, 14H), 1.17-0.98 (m, 2H). ¹⁹F NMR (376 MHz, METHANOL-d4) δ=−120.03-−121.23 (m, 1F).

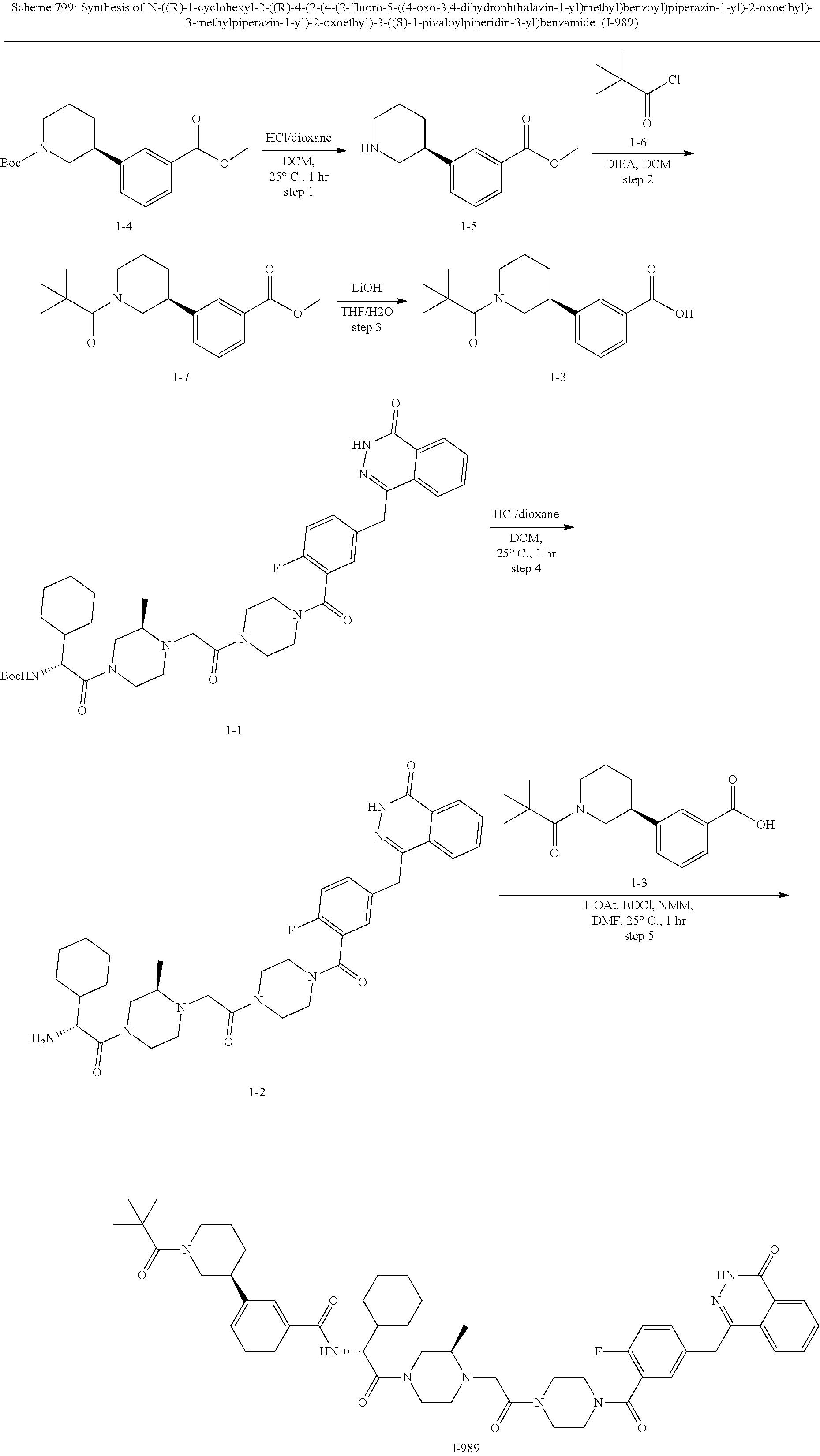

### Step 1: Synthesis of Intermediate 1-5

To a solution of Intermediate 1-4 (2 g, 6.26 mmol, 1 eq) in DCM (10 mL) was added HCl/dioxane (2 M, 20 mL). The mixture was stirred at 25° C. for 16 hr. The reaction mixture was concentrated under reduced pressure to give Intermediate 1-5 (1.7 g, crude, HCl salt) as a white solid. LCMS (Method E): Rt: 0.383 min, [M+H]⁺=220.2. ¹H NMR (400 MHz, METHANOL-d4) δ=8.07-7.89 (m, 2H), 7.62-7.55 (m, 1H), 7.53-7.42 (m, 1H), 3.91 (s, 3H), 3.50-3.38 (m, 2H), 3.20-3.01 (m, 3H), 2.16-2.02 (m, 2H), 1.88 (br d, J=11.2 Hz, 2H).

### Step 2: Synthesis of Intermediate 1-7

To a solution of Intermediate 1-5 (1.7 g, 6.65 mmol, 1 eq, HCl salt) in DCM (20 mL) was added DIEA (2.58 g, 19.94 mmol, 3.47 mL, 3 eq) and Intermediate 1-6 (1.20 g, 9.97 mmol, 1.23 mL, 1.5 eq). The mixture was stirred at 25° C. for 1 hr. To the reaction mixture was added H₂O (20 mL) and it was extracted with DCM (10 mL*3). The combined organic layers were dried, filtered and concentrated under reduced pressure to give a residue. The residue was purified by flash silica gel chromatography (ISCO; 40 g SepaFlash Silica Flash Column, Eluent of 0˜50% EA/PE). The eluent was concentrated to give Intermediate 1-7 (1.6 g, 5.17 mmol, 77.75% yield, 98% purity) as a white solid. LCMS (Method E): Rt: 0.558 min, [M+H]⁺=304.2. SFC: Rt: 1.067 min, ee value=98% ¹H NMR (400 MHz, METHANOL-d4) δ=7.97-7.85 (m, 2H), 7.60-7.51 (m, 1H), 7.48-7.41 (m, 1H), 4.46 (br d, J=12.4 Hz, 2H), 3.91 (s, 3H), 3.08-2.85 (m, 2H), 2.76 (tt, J=3.4, 11.2 Hz, 1H), 2.13-1.99 (m, 1H), 1.95-1.77 (m, 2H), 1.72-1.56 (m, 1H), 1.30 (s, 9H).

### Step 3: Synthesis of Intermediate 1-3

To a solution of Intermediate 1-7 (1.6 g, 5.27 mmol, 1 eq) in THF (6 mL), MeOH (6 mL) and H₂O (6 mL) was added LiOH·H₂O (885.21 mg, 21.09 mmol, 4 eq). The mixture was stirred at 25° C. for 1 hr. To the reaction mixture was added to H₂O (15 mL). The aqueous layer was acidified to pH=2 with conc. HCl and extracted with EA (5 mL*3). The organic phase was dried, filtered and concentrated under reduced pressure to give Intermediate 1-3 (1.4 g, 4.74 mmol, 89.91% yield, 98% purity) as a colorless oil. LCMS (Method E): Rt: 0.491 min, [M+H]⁺=290.3. SFC: Rt: 1.406 min, ee value >99%. ¹H NMR (400 MHz, DMSO-d6) δ=13.15-12.79 (m, 1H), 7.86-7.79 (m, 2H), 7.57-7.50 (m, 1H), 7.49-7.42 (m, 1H), 4.30 (br t, J=12.8 Hz, 2H), 2.98-2.80 (m, 2H), 2.74-2.63 (m, 1H), 2.02-1.88 (m, 1H), 1.84-1.69 (m, 2H), 1.55-1.39 (m, 1H), 1.29-1.18 (m, 9H).

### Step 4: Synthesis of Intermediate 1-2

To a solution of Intermediate 1-1 (60 mg, 80.44 μmol, 1 eq) in DCM (0.3 mL) was added HCl/dioxane (2 M, 0.6 mL, 14.92 eq). The mixture was stirred at 25° C. for 1 hr. The reaction mixture was concentrated under reduced pressure to remove solvent to give Intermediate 1-2 (60 mg, crude, HCl salt) as a white solid and it was used in the next step without further purification. LCMS (Method E): Rt: 0.387 min, [M+H]⁺=646.4.

### Step 5: Synthesis of I-989

To a solution of Intermediate 1-2 (60 mg, 92.91 μmol, 1 eq) in DMF (0.6 mL) was added HOAt (12.65 mg, 92.91 μmol, 13.00 μL, 1 eq), EDCI (53.43 mg, 278.74 μmol, 3 eq), NMM (75.18 mg, 743.30 μmol, 81.72 μL, 8 eq) and Intermediate 1-3 (29.57 mg, 102.20 μmol, 1.1 eq). The mixture was stirred at 25° C. for 1 hr. To the reaction mixture was added to H₂O (1 mL) and it was extracted with EA (1 mL*3). The organic phase was dried, filtered and concentrated under reduced pressure to give a crude product. The crude product combined with another lot of crude material and purified by Prep-HPLC (column: Phenomenex luna C18 150*25 mm*10 um; mobile phase: [water (FA)-ACN]; gradient: 25%-55% B over 10 min). The eluent was concentrated and lyophilized to give I-989 (53 mg, 57.79 μmol, 62.20% yield, 100% purity) as a white solid. LCMS (Method E): Rt: 0.511 min, [M+H]⁺=917.5. SFC: Rt: 1.056 min, de value >99%. ¹H NMR (400 MHz, METHANOL-d4) δ=8.37 (br d, J=7.8 Hz, 1H), 7.98-7.92 (m, 1H), 7.91-7.78 (m, 2H), 7.78-7.67 (m, 2H), 7.53-7.34 (m, 4H), 7.16 (t, J=9.0 Hz, 1H), 4.58 (s, 1H), 4.53-4.42 (m, 2H), 4.38 (s, 2H), 4.12-3.92 (m, 2H), 3.90-3.46 (m, 8H), 3.22-3.05 (m, 2H), 3.04-2.66 (m, 5H), 2.64-2.28 (m, 2H), 2.11-2.00 (m, 1H), 1.99-1.48 (m, 10H), 1.33-1.20 (m, 12H), 1.19-1.03 (m, 5H). ¹⁹F NMR (377 MHz, METHANOL-d4) δ=−118.62-−123.30 (m, 1F).

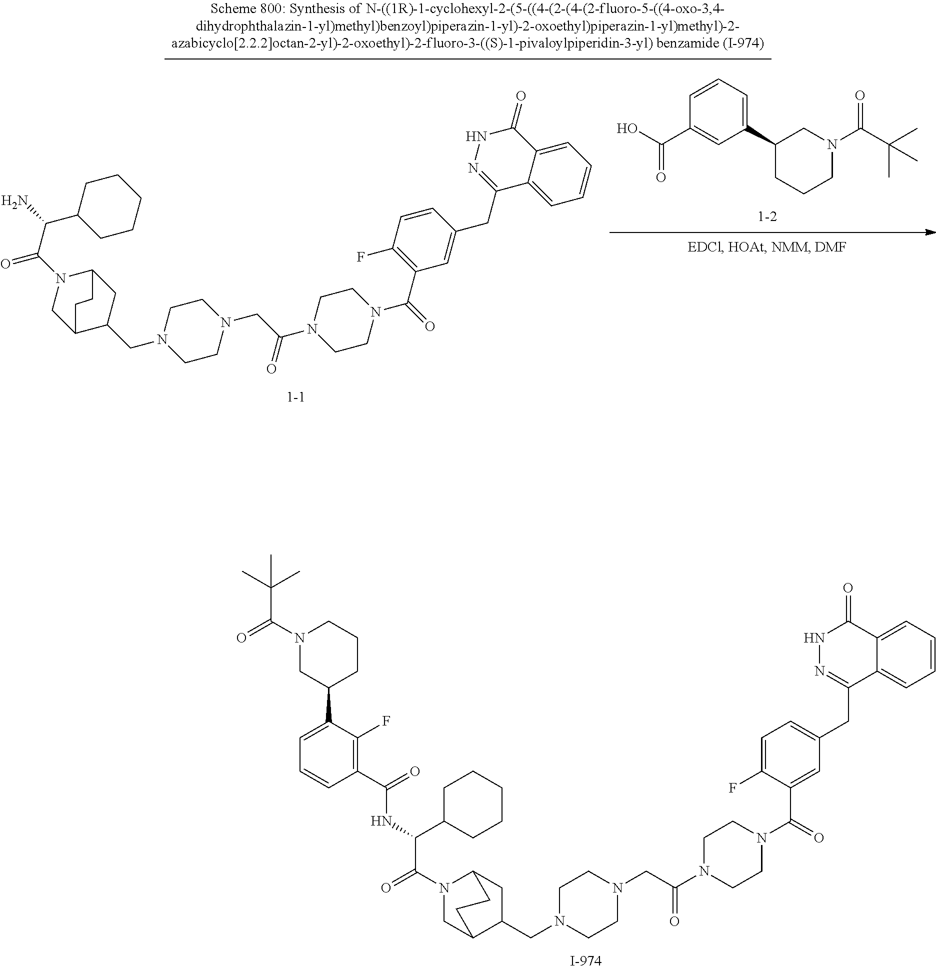 [SMILES: N[C@@H](C(=O)N1CC2CCC1CC2CN1CCN(CC(=O)N2CCN(C(=O)c3cc(Cc4n[nH]c(=O)c5ccccc45)ccc3F)CC2)CC1)C1CCCCC1 ; CC(C)(C)C(=O)N1CCC[C@@H](c2cccc(C(=O)O)c2)C1 ; CC(C)(C)C(=O)N1CCC[C@@H](c2cccc(C(=O)N[C@@H](C(=O)N3CC4CCC3CC4CN3CCN(CC(=O)N4CCN(C(=O)c5cc(Cc6n[nH]c(=O)c7ccccc67)ccc5F)CC4)CC3)C3CCCCC3)c2F)C1]

To a mixture of Intermediate 1-1 (400 mg, 505.44 μmol, 1 eq, HCl salt), NMM (260.00 mg, 2.57 mmol, 282.61 μL, 5.09 eq), 3-[(3S)-1-(2,2-dimethylpropanoyl)-3-piperidyl]-2-fluoro-benzoic acid (160.00 mg, 520.56 μmol, 1.03 eq) and EDCI (300.00 mg, 1.56 mmol, 3.10 eq) in DMF (4 mL) was added HOAt (80.00 mg, 587.76 μmol, 82.22 μL, 1.16 eq) and then the mixture was stirred at 25° C. for 1 hr. The reaction mixture was concentrated under reduced pressure to remove solvent. The residue was diluted with water (50 mL) and extracted with EtOAc (20 mL*2). The combined organic layers were washed with brine (50 mL*2), dried over Na₂SO₄, filtered and concentrated under reduced pressure to give a residue. The crude product was purified by reversed-phase HPLC (0.1% FA condition) to give I-974 (259.75 mg, 247.36 mol, 48.94% yield, 99.45% purity) as a white solid. LCMS (Method x): Retention time=0.504 min, (M+H)=1045.6.1H NMR (400 MHz, DMSO-d6) δ=12.60 (d, J=1.6 Hz, 1H), 8.50-8.34 (m, 1H), 8.26 (br d, J=8.0 Hz, 1H), 8.16 (s, 1H), 8.00-7.93 (m, 1H), 7.93-7.78 (m, 2H), 7.52-7.33 (m, 4H), 7.28-7.18 (m, 2H), 4.70-4.45 (m, 1H), 4.33 (br s, 4H), 4.20-3.96 (m, 1H), 3.86-3.54 (m, 4H), 3.54-3.43 (m, 3H), 3.27-3.04 (m, 6H), 2.96-2.80 (m, 3H), 2.45-2.11 (m, 9H), 1.99-1.40 (m, 18H), 1.23-1.17 (m, 9H), 1.14 (br d, J=2.0 Hz, 3H), 1.07-0.91 (m, 2H). ¹⁹F NMR (377 MHz, DMSO-d6) δ=−119.78 (br s, 1F), −120.15-−120.48 (m, 1F). SFC: Retention time=1.939 min, 2.057 min, 2.512 min.

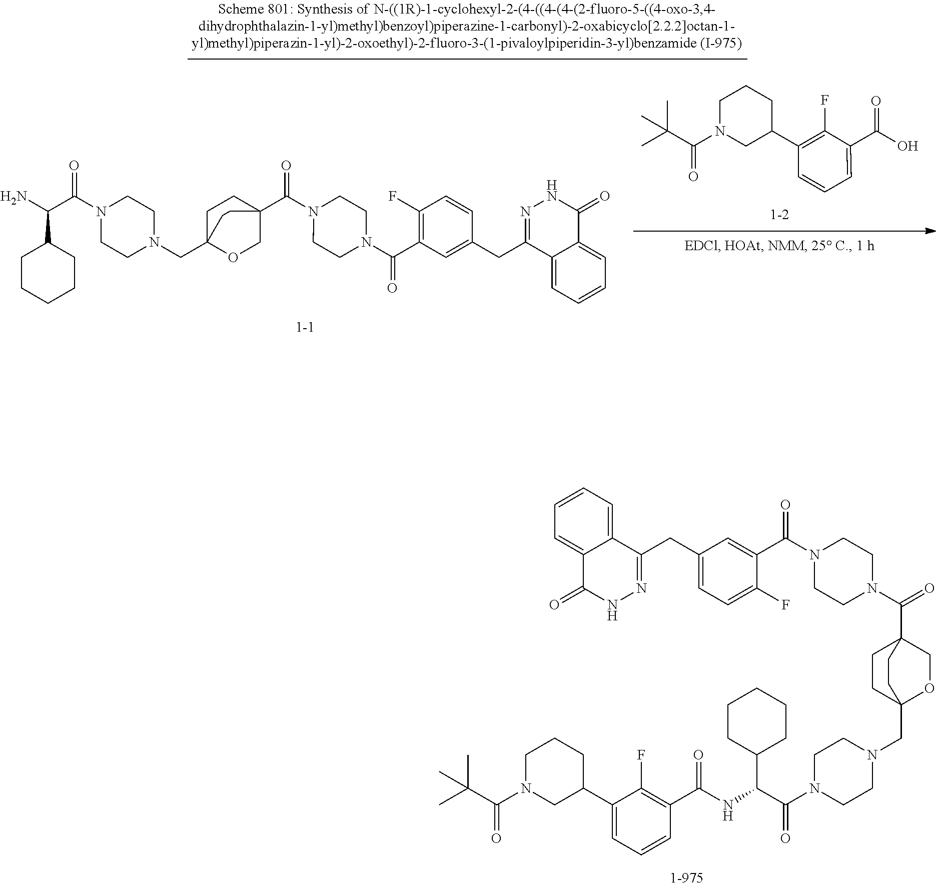 [SMILES: N[C@@H](C(=O)N1CCN(CC23CCC(C(=O)N4CCN(C(=O)c5cc(Cc6n[nH]c(=O)c7ccccc67)ccc5F)CC4)(CC2)CO3)CC1)C1CCCCC1 ; CC(C)(C)C(=O)N1CCCC(c2cccc(C(=O)O)c2F)C1 ; CC(C)(C)C(=O)N1CCCC(c2cccc(C(=O)N[C@@H](C(=O)N3CCN(CC45CCC(C(=O)N6CCN(C(=O)c7cc(Cc8n[nH]c(=O)c9ccccc89)ccc7F)CC6)(CC4)CO5)CC3)C3CCCCC3)c2F)C1]

To a mixture of Intermediate 1-2 (207.15 mg, 673.95 μmol, 1 eq), Intermediate 1-1 (0.5 g, 673.95 μmol, 1 eq), EDCI (387.59 mg, 2.02 mmol, 3 eq) and NMM (545.35 mg, 5.39 mmol, 592.77 μL, 8 eq) in DMF (5 mL) was added HOAt (91.73 mg, 673.95 μmol, 94.28 μL, 1 eq) and then the mixture was stirred at 25° C. for 1 hr. The reaction mixture was quenched with water (20 mL) and extracted with EA (10 mL*3), the combined the organic phase was washed by aqueous NaCl (20 mL*3) and dried by anhydrous Na₂SO₄, and concentrated to give crude product. The crude product was purified by reverse phase chromatography (105 g of XB—C18, 20-40 μm, 120 Å) Mobile phase: A for H₂O (0.1% FAv/v) and B for acetonitrile; Gradient: B30%-50% in 30 min; Flow rate: 100 ml/min; Column temperature: R.T. Wavelength: 220 nm/254 nm), and the eluent was concentrated to remove organic solvents and lyophilized to give product. The product was further purified by normal prep-HPLC (column: Welch Ultimate XB—CN 250*50*10 um;mobile phase: [Hexane-EtOH (0.1% NH₃.H₂O)]; gradient: 25%-65% B over 15 min). I-975 (252.1 mg, 244.46 mol, 36.27% yield, 100% purity) was obtained as a white solid. LCMS (Method E): Retention time: 0.520 min, (M+H)=1031.5. SFC: Retention time: 3.337 min, 4.931 min. ¹H NMR (400 MHz, DMSO-d6) δ=12.59 (s, 1H), 8.44-8.34 (m, 1H), 8.26 (d, J=8.0 Hz, 1H), 8.00-7.93 (m, 1H), 7.93-7.80 (m, 2H), 7.52-7.38 (m, 3H), 7.36 (d, J=6.4 Hz, 1H), 7.28-7.20 (m, 2H), 4.76 (br s, 1H), 4.41-4.26 (m, 4H), 3.80 (br s, 2H), 3.59 (br s, 6H), 3.47 (d, J=2.4 Hz, 4H), 3.15 (d, J=1.0 Hz, 2H), 2.96-2.80 (m, 3H), 2.41 (d, J=4.0 Hz, 2H), 2.22 (br s, 2H), 2.13-2.02 (m, 2H), 1.92-1.64 (m, 11H), 1.63-1.46 (m, 5H), 1.28-1.19 (m, 11H), 1.15 (d, J=9.6 Hz, 2H), 1.09-1.00 (m, 2H), 0.89-0.83 (m, 1H). ¹⁹F NMR (377 MHz, DMSO-d6) δ=−119.69 (s, 1F), −119.94-−120.41 (m, 1F).

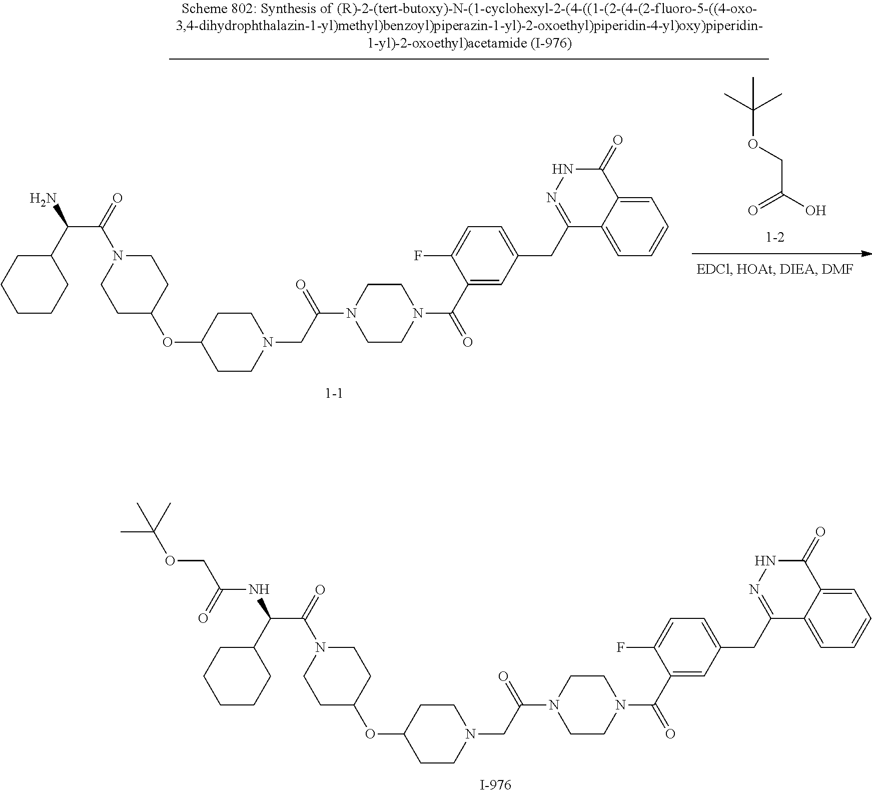

To a mixture of Intermediate 1-2 (68.98 mg, 521.96 μmol, 1 eq), Intermediate 1-1 (0.4 g, 521.96 μmol, 1 eq, HCl salt), EDCI (300.18 mg, 1.57 mmol, 3 eq) and NMM (422.36 mg, 4.18 mmol, 459.08 μL, 8 eq) in DMF (4 mL) was added HOAt (71.04 mg, 521.96 μmol, 73.02 μL, 1 eq) and then the mixture was stirred at 25° C. for 1 hr. The reaction mixture was diluted with water (10 mL) and extracted with EtOAc (10 mL*3). The combined organic layers were washed with aqueous NaCl (10 mL), dried over anhydrous Na₂SO₄, filtered and concentrated under reduced pressure to give a residue. The residue was purified by normal phase prep-HPLC (column: Welch Ultimate XB—CN 250*50*10 um;mobile phase: [Hexane-EtOH (0.1% NH3·H₂O)]; gradient: 20%-60% B over 15 min ]; gradient: 20%-60% B over 15 min), and the target peak was concentrated and lyophilized to afford desired product. The product was purified by normal phase prep-HPLC (column: Welch Ultimate XB—CN 250*50*10 um; mobile phase: [Hexane-EtOH]; gradient: 20%-60% B over 15 min), and the target peak was concentrated and lyophilized to afford the desired product. I-976 (152.72 mg, 179.19 μmol, 34.33% yield, 99.03% purity) as a yellow solid was obtained. LCMS (Method X): Retention time: 1.126 min, (M+H)=844.7. SFC: Retention time: 1.700 min. ¹H NMR (400 MHz, DMSO-d6) δ=12.59 (s, 1H), 8.26 (d, J=7.6 Hz, 1H), 8.00-7.93 (m, 1H), 7.89 (t, J=7.2 Hz, 1H), 7.86-7.80 (m, 1H), 7.43-7.42 (m, 1H), 7.37 (d, J=8.4 Hz, 2H), 7.24 (t, J=9.2 Hz, 1H), 4.76-4.63 (m, 1H), 4.33 (s, 2H), 3.95-3.71 (m, 4H), 3.69-3.53 (m, 4H), 3.52-3.36 (m, 4H), 3.20-2.99 (m, 5H), 2.73-2.59 (m, 2H), 2.20-2.03 (m, 2H), 1.83-1.52 (m, 10H), 1.48-1.20 (m, 5H), 1.16 (s, 10H), 1.12-0.84 (m, 4H). ¹⁹F NMR (376 MHz, DMSO-d6) δ=−119.78 (br s, 1F).

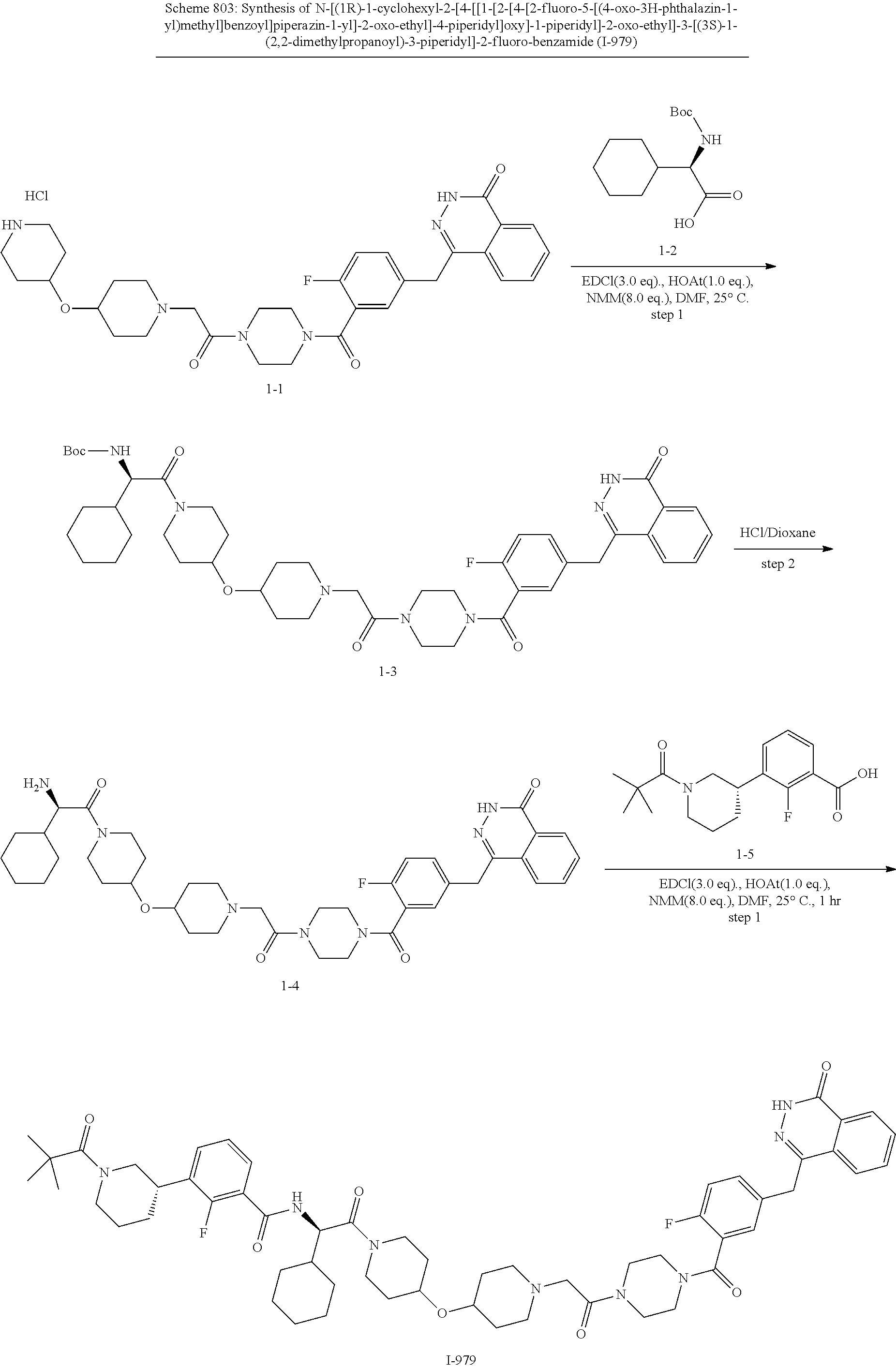

### Step 1: Synthesis of Intermediate 1-3

To a solution of Intermediate 1-1 (2.85 g, 4.82 mmol, 1 eq) in DMF (35 mL) was added Intermediate 1-2 (1.24 g, 4.82 mmol, 1 eq), EDCI (2.77 g, 14.47 mmol, 3 eq), HOAt (656.72 mg, 4.82 mmol, 674.94 μL, 1 eq) and NMM (3.90 g, 38.60 mmol, 4.24 mL, 8 eq). The resulting mixture was stirred at 25° C. for 1 hr. The reaction mixture was diluted by water (100 mL) and extracted by EA (30 mL*3). The organic phase was combined and concentrated to afford the crude. The crude was purified by prep-HPLC (FA condition). The eluent was combined and concentrated to afford the target product. After purification, Intermediate 1-3 (1.65 g, 1.97 mmol, 40.92% yield, 99.32% purity) was obtained as white solid. LCMS (Method E): Retention time=0.516 min, (M+H)⁺=830.6. ¹H NMR (400 MHz, CHLOROFORM-d) δ=10.97 (d, J=17.6 Hz, 1H), 8.52-8.43 (m, 1H), 7.83-7.66 (m, 3H), 7.41-7.29 (m, 2H), 7.04 (t, J=8.8 Hz, 1H), 5.37 (d, J=8.4 Hz, 1H), 4.47 (d, J=6.4 Hz, 1H), 4.29 (s, 2H), 3.97-3.53 (m, 9H), 3.47-3.12 (m, 7H), 2.74 (s, 2H), 2.33-2.13 (m, 2H), 1.94-1.69 (m, 8H), 1.66-1.53 (m, 6H), 1.41 (s, 9H), 1.22-0.94 (m, 5H). ¹⁹F NMR (376 MHz, CHLOROFORM-d) δ=−117.67 (br d, J=38.1 Hz, 1F).

### Step 2: Synthesis of Intermediate 1-4

A solution of Intermediate 1-3 (1.65 g, 1.99 mmol, 1 eq) in HCl/dioxane (20 mL) was stirred at 25° C. for 1 hr. The reaction mixture was concentrated under reduced pressure to afford the crude. After work-up, Intermediate 1-4 (1.89 g, crude) was obtained as a white solid, which was used directly in the next step. LCMS (Method E): Retention time=0.403 min, (M+H)+=730.5.

### Step 3: Synthesis of I-979

To a solution of Intermediate 1-4 (0.5 g, 652.45 μmol, 1 eq, HCl salt) in DMF (10 mL) was added EDCI (375.23 mg, 1.96 mmol, 3 eq), HOAt (88.81 mg, 652.45 μmol, 91.27 μL, 1 eq), Intermediate 1-5 (200.54 mg, 652.45 μmol, 1 eq) and NMM (527.95 mg, 5.22 mmol, 573.86 μL, 8 eq). The resulting mixture was stirred at 25° C. for 1 hr. The reaction mixture was diluted with water (50 mL) then extracted by EA (20 mL*3). The organic phase was combined and concentrated to afford the crude. The crude was purified via prep-HPLC (FA condition, column: Phenomenex luna C18 150*25 mm*10 um; mobile phase: [water (FA)-ACN]; gradient: 25%-55% B over 10 min). The eluent was combined and lyophilized to yield the target product. After purification, I-979 (259.1 mg, 253.41 μmol, 38.84% yield, 99.684% purity) was obtained as a white solid. LCMS (Method E): Retention time=0.514 min, (M+H)⁺=1019.8. SFC: Retention time=269.7 min. ¹H NMR (400 MHz, DMSO-d6) δ=12.59 (s, 1H), 8.45-8.34 (m, 1H), 8.26 (d, J=6.4 Hz, 1H), 8.00-7.94 (m, 1H), 7.92-7.78 (m, 2H), 7.54-7.33 (m, 4H), 7.28-7.18 (m, 2H), 4.87-4.67 (m, 1H), 4.33 (s, 4H), 4.07-3.77 (m, 2H), 3.72-3.39 (m, 9H), 3.26-2.96 (m, 7H), 2.95-2.77 (m, 3H), 2.74-2.57 (m, 2H), 2.22-2.02 (m, 2H), 1.88-1.58 (m, 12H), 1.52-1.32 (m, 4H), 1.20 (s, 9H), 1.18-0.93 (m, 5H). ¹⁹F NMR (376 MHz, DMSO-d6) δ=−119.78 (br s, 1F), −120.32 (br d, J=24.7 Hz, 1F).

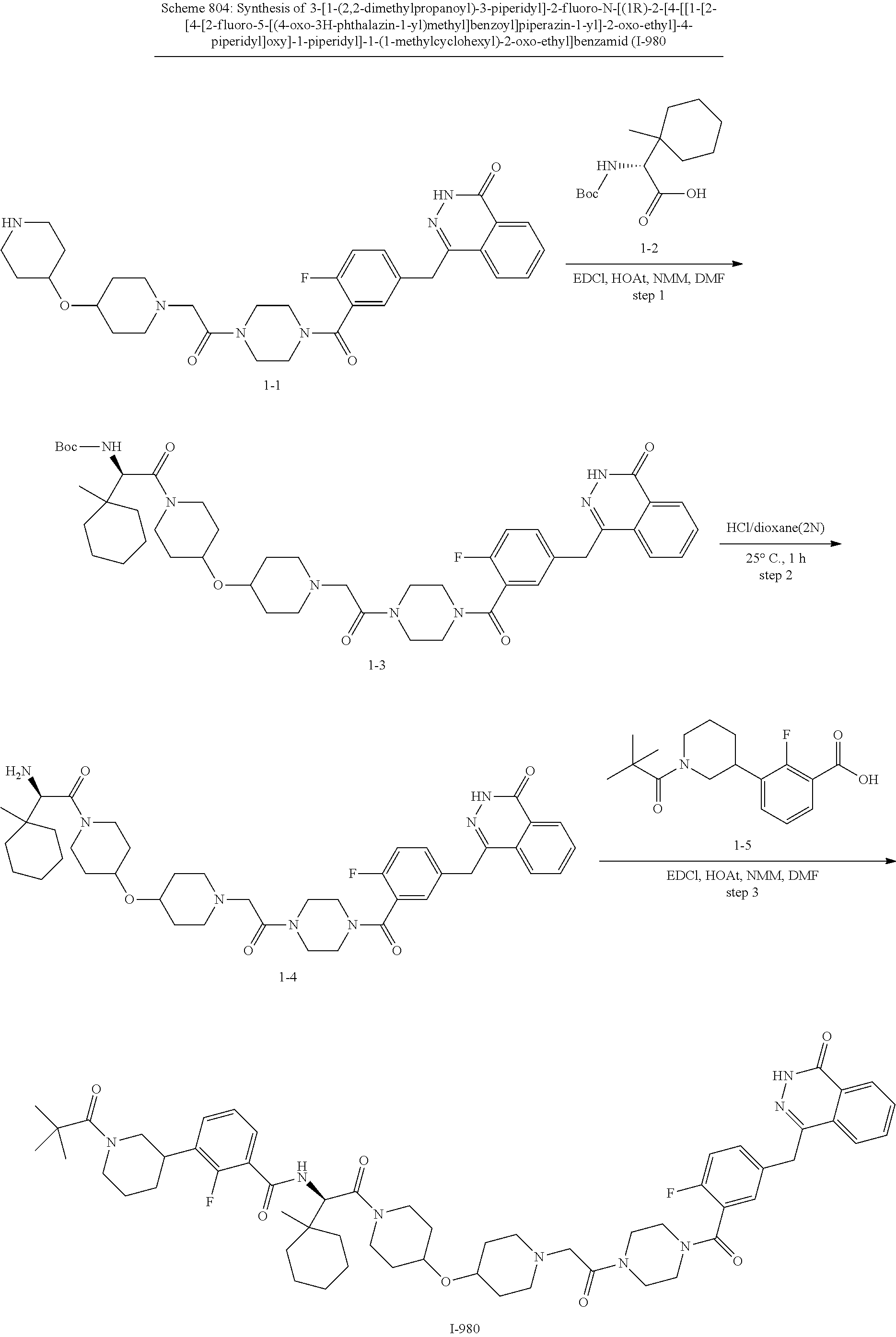

### Step 1: Synthesis of Intermediate 1-3

To a solution of Intermediate 1-2 (151.44 mg, 558.08 μmol, 1 eq) in DMF (3.5 mL) was added EDCI (320.95 mg, 1.67 mmol, 3 eq), HOAt (113.94 mg, 837.12 μmol, 117.10 μL, 1.5 eq), NMM (282.24 mg, 2.79 mmol, 306.78 μL, 5 eq) and Intermediate 1-1 (350 mg, 558.08 μmol, 1 eq, HCl salt), and the reaction mixture was stirred at 25° C. for 0.5 h. To the mixture was added water (10 mL) and it was extracted with EA (5 mL*3), the combined organic phase was washed by brine (10 mL*2), then dried over Na₂SO₄, filtered and concentrated under reduced pressure to give the residue. The residue was purified by reverse phase chromatography (120 g of XB—C18, 20-40 μm, 120 Å) Mobile phase: A for H₂O (0.1% NH₃·H₂Ov/v) and B for acetonitrile; Gradient: B60%-80% in 10 min; Flow rate: 100 ml/min; Column temperature: R.T. Wavelength: 220 nm/254 nm), and then the eluent was concentrated to remove organic solvents and lyophilized to give Intermediate 1-3 (270 mg, 319.90 μmol, 57.32% yield) as a white solid. LCMS (Method G): Rt=0.694 min, [M+H]⁺=844.6. SFC: Rt=1.635 min. ¹H NMR (400 MHz, DMSO-d6) δ=12.59 (s, 1H), 8.26 (d, J=7.6 Hz, 1H), 8.02-7.93 (m, 1H), 7.92-7.79 (m, 2H), 7.48-7.34 (m, 2H), 7.23 (t, J=8.8 Hz, 1H), 6.59-6.39 (m, 1H), 4.43-4.37 (m, 1H), 4.33 (s, 2H), 4.08-3.73 (m, 2H), 3.69-3.54 (m, 4H), 3.51-3.44 (m, 2H), 3.23-3.04 (m, 7H), 2.18-2.07 (m, 2H), 1.85-1.69 (m, 4H), 1.57-1.08 (m, 26H), 0.93 (s, 3H). ¹⁹F NMR (377 MHz, DMSO-d6) δ=−119.79 (br s, 1F).

### Step 2: Synthesis of Intermediate 1-4

To a solution of intermediate 1-3 (270 mg, 319.90 μmol, 1 eq) in DCM (1 mL) was added HCl/dioxane (2 mL, 2 M), and the reaction mixture was stirred at 25° C. for 1 h. The reaction mixture was concentrated to give Intermediate 1-4 (250 mg, crude, HCl salt) as a white solid. LCMS (Method E): Rt=0.412 min, [M+H]⁺=744.6. SFC: Rt=1.632 min, ee value >99%. ¹H NMR (400 MHz, DMSO-d6) δ=12.61 (d, J=3.8 Hz, 1H), 9.86-9.54 (m, 1H), 8.26 (d, J=7.6 Hz, 1H), 8.14 (br s, 2H), 8.01-7.80 (m, 3H), 7.51-7.44 (m, 1H), 7.41-7.31 (m, 1H), 7.25 (t, J=9.2 Hz, 1H), 4.47-4.27 (m, 4H), 4.22-3.92 (m, 2H), 3.87-3.64 (m, 6H), 3.39-2.89 (m, 9H), 2.30-1.68 (m, 7H), 1.63-1.20 (m, 13H), 1.11-0.93 (m, 4H). ¹⁹F NMR (377 MHz, DMSO-d6) δ=−119.69 (br d, J=6.7 Hz, 1F).

### Step 3: Synthesis of I-980

Intermediate 1-4 (250 mg, crude, HCl salt) and Intermediate 1-5 (98.47 mg, 320.36 μmol, 1 eq) in DMF (2.5 mL) was added EDCI (184.24 mg, 961.08 μmol, 3 eq), HOAt (65.41 mg, 480.54 μmol, 67.22 L, 1.5 eq), and NMM (162.02 mg, 1.60 mmol, 176.11 μL, 5 eq), and the reaction mixture was stirred at 25° C. for 1 h. To the reaction mixture was added with water (10 mL) and it was extracted with EA (5 mL*3), the combined organic phase was washed by brine (10 mL*2), then dried over Na₂SO₄, filtered and concentrated under reduced pressure to give the residue. The crude product was purified by reverse phase chromatography twice (100 g of XB—C18, 20-40 μm, 120 Å) Mobile phase: A for H₂O (0.1% NH₃·H₂Ov/v) and B for acetonitrile; Gradient: B60%-70% in 10 min; Flow rate: 100 ml/min; Column temperature: R.T. Wavelength: 220 nm/254 nm), and then the eluent was concentrated to remove organic solvents and lyophilized to give I-980 (290 mg, 280.67 μmol, 87.61% yield, 99.2% purity) as a white solid. LCMS (Method E): Rt=0.741 min, [M+H]⁺=1033.8. SFC: Rt=2.681 min, 3.769 min ¹H NMR (400 MHz, DMSO-d6) δ=12.59 (s, 1H), 8.31-8.12 (m, 2H), 8.00-7.93 (m, 1H), 7.92-7.79 (m, 2H), 7.53-7.34 (m, 4H), 7.27-7.18 (m, 2H), 5.00 (t, J=7.2 Hz, 1H), 4.44-4.24 (m, 4H), 4.08-3.75 (m, 2H), 3.72-3.54 (m, 4H), 3.53-3.39 (m, 4H), 3.26-3.03 (m, 5H), 3.01-2.75 (m, 4H), 2.67 (d, J=1.4 Hz, 2H), 2.19-2.05 (m, 2H), 1.94-1.70 (m, 7H), 1.58-1.29 (m, 14H), 1.20 (br s, 9H), 1.13 (d, J=8.4 Hz, 1H), 1.03 (s, 3H). ¹⁹F NMR (376 MHz, DMSO-d6) δ=−119.77 (br s, 1F), −120.17 (br d, J=13.5 Hz, 1F).

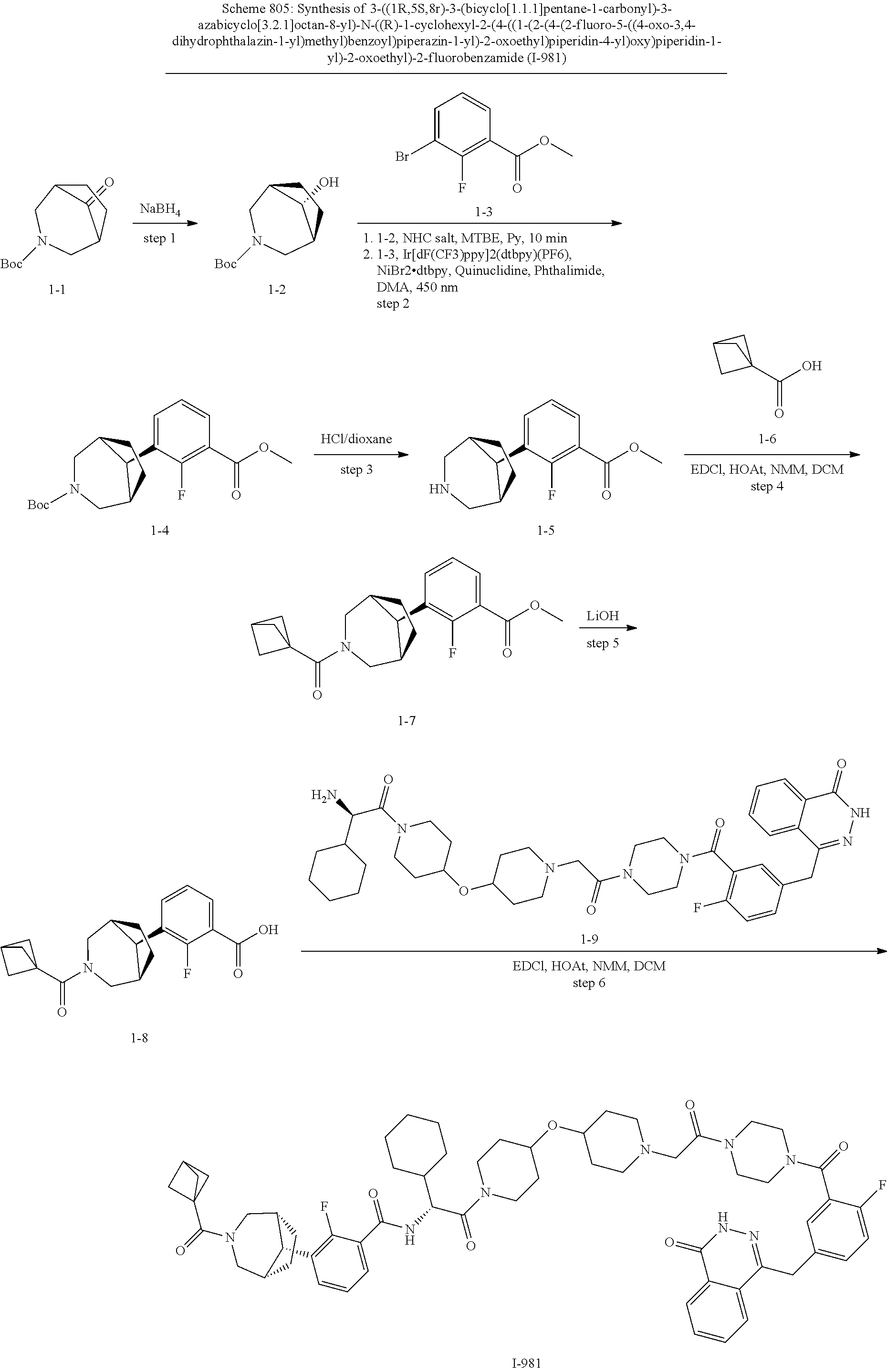

### Step 1: Synthesis of Intermediate 1-2

To a solution of Intermediate 1-1 (9 g, 39.95 mmol, 1 eq) in MeOH (90 mL) was added NaBH₄ (2.27 g, 59.92 mmol, 1.5 eq) at 0° C. Then the mixture was stirred at 0° C. for 1 hr. The reaction mixture was poured into water (200 mL) and extracted with EA (200 mL*2), the organic layer was washed with brine (200 mL) and dried over Na₂SO₄, then concentrated to give Intermediate 1-2 (9 g, 39.60 mmol, 99.11% yield, 100% purity) as a white solid. LCMS (Method E): Rt=0.469 min, [M+Na]⁺=172.2. H NMR (400 MHz, DMSO-d6) δ=4.06-4.04 (m, 1H), 3.70 (br d, J=11.6 Hz, 1H), 3.56 (br d, J=12.0 Hz, 1H), 3.43-3.26 (m, 2H), 2.06-1.95 (m, 2H), 1.75-1.57 (m, 4H), 1.48 (s, 9H).

### Step 2: Synthesis of Intermediate 1-4

An oven-dried 8 mL vial was charged with Intermediate 1-2 (3 g, 13.20 mmol, 1 eq) and 5,7-ditert-butyl-3-phenyl-1,3-benzoxazol-3-ium;tetrafluoroborate (8.35 g, 21.12 mmol, 1.6 eq). After the vial was vacuumed and refilled with nitrogen gas twice, MTBE (20 mL) was added and the reaction was stirred at 25° C. for 5 min. Then, a solution of pyridine (1.67 g, 21.12 mmol, 1.70 mL, 1.6 eq) in MTBE (5 mL) was added dropwise at 25° C. over the course of 2 min. The resulting solution was stirred at 25° C. for 10 min. A white solid precipitated out during this time. Another oven-dried 15 mL vial was charged with bis[3,5-difluoro-2-[5-(trifluoromethyl)-2-pyridyl]phenyl]iridium (1+); 4-tert-butyl-2-(4-tert-butyl-2-pyridyl)pyridine;hexafluorophosphate (222.11 mg, 197.98 umol, 0.015 eq), 4-tert-butyl-2-(4-tert-butyl-2-pyridyl)pyridine; dibromonickel (481.97 mg, 989.88 umol, 0.075 eq), quinuclidine (2.57 g, 23.10 mmol, 1.75 eq), Intermediate 1-3 (3.08 g, 13.20 mmol, 1 eq) and isoindoline-1,3-dione (436.93 mg, 2.97 mmol, 0.225 eq). DMA (25 mL) was added to this vial under an atmosphere of nitrogen. The methyl tert-butyl ether suspension was transferred to a 5 mL syringe under air. Then a syringe filter and new needle were installed on the syringe, before the methyl tertbutyl ether solution was injected through the syringe filter into the dimethylacetamide solution. The reaction mixture was sparged with nitrogen for 15 minutes before sealing with parafilm. The vial was stirred at 1500 rpm stir rate and irradiated under 450 nm LED modules at 100% light intensity with maxed fan speed and 1500 rpm stirring rate in a PennOC Integrated Photoreactor for 16 hours. The reaction mixture was poured into water (200 mL) and extracted with EA (200 mL*2), the organic layer was washed with brine (200 mL) and dried over Na₂SO₄, then concentrated to give the crude product. The crude product was poured into water (200 mL) and extracted with EA (200 mL*2), the combined organic layer was washed with brine (200 mL) and dried over Na₂SO₄, then concentrated to give the crude product. The crude product was purified by flash silica gel chromatography (SiO₂, PE:EA=10:1 to 3:1) to give Intermediate 1-4 (1 g, 2.07 mmol, 15.72% yield, 75.4% purity) as a white solid. LCMS (Method E): Rt=0.615 min, [M+H]⁺=308.2.

### Step 3: Synthesis of Intermediate 1-5

A solution of Intermediate 1-4 (1 g, 2.75 mmol, 1 eq) in HCl/dioxane (10 mL, 2M) was stirred at 25° C. for 1 hr. The reaction mixture was concentrated to give the crude product. The crude product was triturated with EA (20 mL) at 25° C. for 30 min to give Intermediate 1-5 (700 mg, 2.07 mmol, 75.36% yield, 88.8% purity, HCl salt) as a white solid. LCMS (Method E): Rt=0.387 min, [M+H]⁺=264.2.

### Step 4: Synthesis of Intermediate 1-7

To a solution of Intermediate 1-6 (82.29 mg, 733.90 umol, 1.1 eq), EDCI (255.80 mg, 1.33 mmol, 2 eq), HOAt (45.41 mg, 333.59 umol, 0.5 eq), and NMM (337.42 mg, 3.34 mmol, 5 eq) in DCM (5 mL) was added Intermediate 1-5 (200 mg, 667.18 umol, 1 eq, HCl salt). Then the mixture was stirred at 25° C. for 1 hr. The reaction mixture was poured into water (20 mL) and extracted with DCM (20 mL*2), the organic layer was washed with brine (20 mL) and dried over Na₂SO₄, then concentrated to give the crude product. The crude product was purified by flash silica gel column chromatography (SiO₂, PE:EA=10:1 to 3:1) to give Intermediate 1-7 (230 mg, 636.43 umol, 95.39% yield, 98.9% purity) as a colorless oil. LCMS (Method E): Rt=0.578 min, [M+H]⁺=358.1.

### Step 5: Synthesis of Intermediate 1-8

To a solution of Intermediate 1-7 (220 mg, 636.43 umol, 95.39% yield, 98.9% purity) in MeOH (3 mL) and H₂O (3 mL) was added LiOH·H₂O (129.15 mg, 3.08 mmol, 5 eq). Then the mixture was stirred at 25° C. for 1 hr. The reaction mixture was poured into water (20 mL) and adjusted to pH=2 with 1N aq·HCl, and extracted with EA (20 mL*2). The organic layer was washed with brine (20 mL) and dried over Na₂SO₄, then concentrated to give Intermediate 1-8 (150 mg, 435.07 umol, 70.68% yield, 99.6% purity)) as a white solid. LCMS (Method E): Rt=0.478 min, [M+H]⁺=344.1.

### Step 6: Synthesis of I-981

To a solution of Intermediate 1-9 (167.38 mg, 218.65 umol, 1 eq, HCl salt), EDCI (85.75 mg, 447.30 umol, 2 eq), HOAt (15.22 mg, 111.83 umol, 0.5 eq), and NMM (113.11 mg, 1.12 mmol, 5 eq) in DCM (1 mL) was added Intermediate 1-8 (75 mg, 218.65 umol, 1 eq). Then the mixture was stirred at 25° C. for 1 hr. The reaction mixture was poured into water (20 mL) and extracted with DCM (20 mL*2), the organic layer was washed with brine (20 mL) and dried over Na₂SO₄, then concentrated to give the crude product. The crude product was purified by prep-HPLC (column: Waters Xbridge 150*25 mm*5 um; mobile phase: [water (NH₄HCO₃)-ACN]; gradient: 18%-48% B over 15 min). The eluent was lyophilized to give I-981 (33.26 mg, 30.08 umol, 13.77% yield, 99.61% purity, FA salt) as a white solid. LCMS (Method E): Rt=0.522 min, [M+H]⁺=1055.6. ¹H NMR (400 MHz, DMSO-d6) δ=12.60 (s, 1H), 8.44-8.30 (m, 1H), 8.27 (br d, J=8.0 Hz, 1H), 8.18 (s, 1H), 8.02-7.94 (m, 1H), 7.93-7.79 (m, 2H), 7.49-7.30 (m, 4H), 7.28-7.15 (m, 2H), 4.87-4.77 (m, 1H), 4.34 (s, 2H), 4.23 (br d, J=11.2 Hz, 1H), 4.10-3.84 (m, 4H), 3.64 (br s, 5H), 3.55-3.22 (m, 7H), 3.13-2.98 (m, 2H), 2.84 (br d, J=12.8 Hz, 1H), 2.68 (br s, 2H), 2.58-2.52 (m, 2H), 2.47 (s, 1H), 2.20-2.06 (m, 8H), 1.86-1.57 (m, 12H), 1.49-1.25 (m, 6H), 1.23-0.97 (m, 5H).

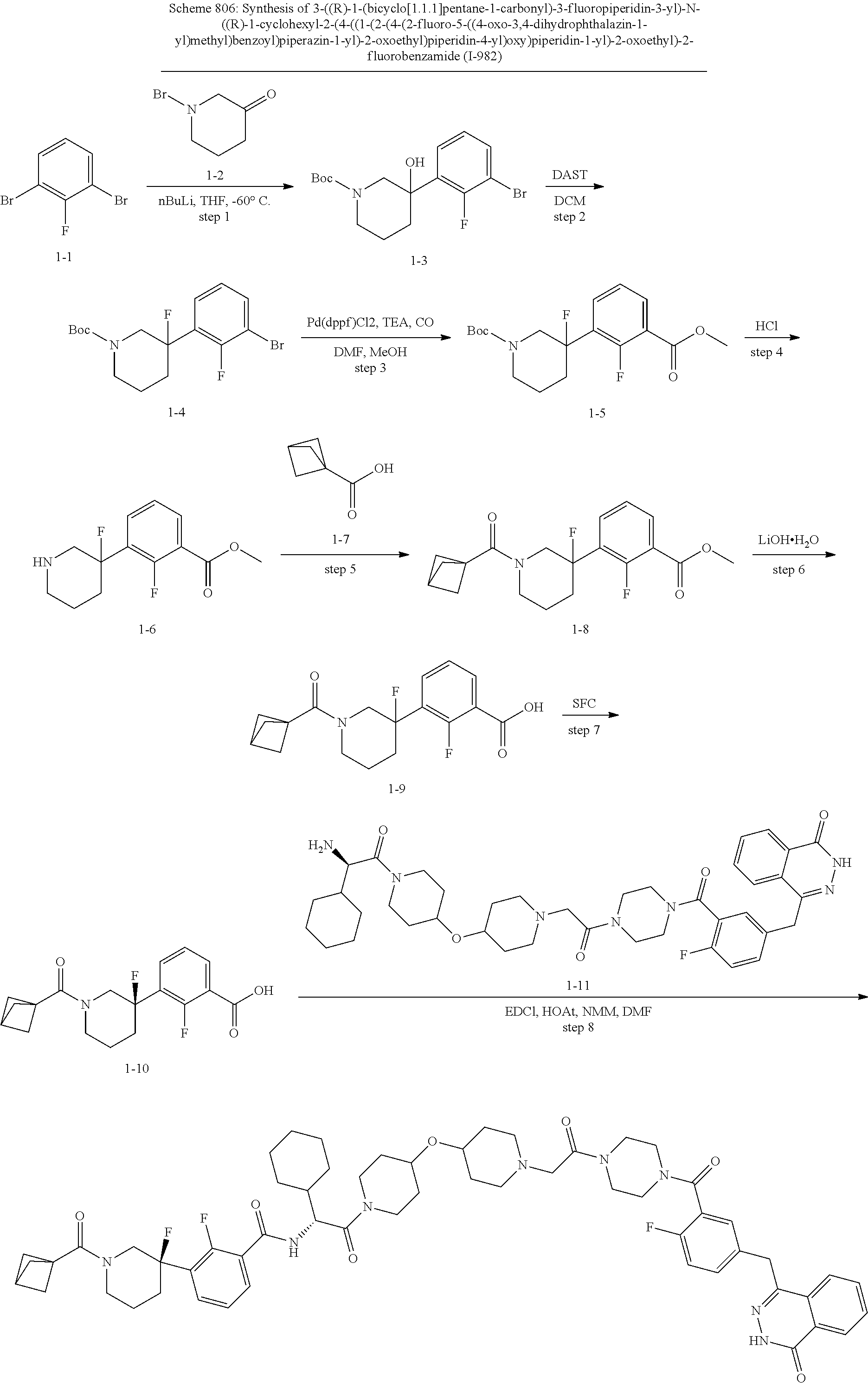

### Step 1: Synthesis of Intermediate 1-3

A solution of Intermediate 1-1 (9.5 g, 37.42 mmol, 1 eq) in THF (100 mL) was added n-BuLi (2.5 M, 14.97 mL, 1 eq) at −65° C. under N₂ and it was stirred at −65° C. for 1 hr. Then to the mixture was added Intermediate 1-2 (8.20 g, 41.16 mmol, 1.1 eq) at −65° C. and it was stirred at −65° C. for 1 hr. The reaction mixture was poured into aq·NH₄Cl (200 mL), and extracted with EA (200 mL*2), the organic layer was washed with brine (200 mL) and dried over Na₂SO₄, and concentrated to give the crude product. The crude product was purified by flash silica gel chromatography (SiO₂, PE:EA=10:1 to 3:1) to give Intermediate 1-3 (9.1 g, 22.47 mmol, 60.05% yield, 92.4% purity) as yellow oil. LCMS (Method E): Rt=0.582 min, [M+Na]⁺=302.1.

### Step 2: Synthesis of Intermediate 1-4

To a solution of Intermediate 1-3 (9 g, 24.05 mmol, 1 eq) in DCM (100 mL) was added DAST (5.81 g, 36.07 mmol, 4.77 mL, 1.5 eq) at −65° C. Then the mixture was stirred at −65° C. for 1 hr. The reaction mixture was poured into aq·Na₂CO₃ (200 mL) and extracted with DCM (200 mL*2), the organic layer was washed with brine (200 mL) and dried over Na₂SO₄, then concentrated to give the crude product. The crude product was purified by reverse phase column chromatography (FA) to give Intermediate 1-4 (4.2 g, 10.73 mmol, 44.61% yield, 96.1% purity) as a colorless oil. LCMS (Method E): Rt=0.470 min, [M+H]⁺=300.0.

### Step 3: Synthesis of Intermediate 1-5

To a solution of Intermediate 1-4 (4.2 g, 11.16 mmol, 1 eq) and TEA (3.39 g, 33.49 mmol, 4.66 mL, 3 eq) in MeOH (40 mL) and DMF (40 mL) was added Pd(dppf)Cl₂·CH₂Cl₂ (911.63 mg, 1.12 mmol, 0.1 eq). Then the mixture was stirred at 140° C. for 30 min under CO (3 Mpa). The reaction mixture was poured into water (200 mL) and extracted with EA (200 mL*2), and the organic layer was washed with brine (200 mL) and dried over Na₂SO₄, then concentrated to give the crude product. The crude product was purified by flash silica gel column chromatography (SiO₂, PE:EA=10:1 to 3:1) to give Intermediate 1-5 (2.6 g, 7.32 mmol, 65.54% yield, 100% purity) as colorless oil. LCMS (Method E): Rt=0.588 min, [M+H]⁺=280.1. ¹H NMR (400 MHz, DMSO-d6) δ=8.00-7.88 (m, 1H), 7.81-7.79 (m, 1H), 7.32-7.28 (m, 1H), 4.41-4.17 (m, 2H), 3.97-3.95 (m, 3H), 3.59-3.31 (m, 1H), 3.04-2.74 (m, 1H), 2.42-2.28 (m, 1H), 2.06-1.92 (m, 2H), 1.66 (br s, 1H), 1.49 (br d, J=8.0 Hz, 9H).

### Step 4: Synthesis of Intermediate 1-6

A solution of Intermediate 1-5 (2.6 g, 7.32 mmol, 1 eq) in HCl/dioxane (30 mL, 2M) was stirred at 25° C. for 1 hr. The reaction mixture was concentrated to give the crude product. The crude product was triturated with EA (20 mL) at 25° C. for 30 min to give Intermediate 1-6 (2.1 g, 7.17 mmol, 98.0% yield, 99.6% purity, HCl salt) as an off-white solid. LCMS (Method E): Rt=0.359 min, [M+H]⁺=259.1.

### Step 5: Synthesis of Intermediate 1-8

To a solution of Intermediate 1-7 (211.40 mg, 1.89 mmol, 1.1 eq), EDCI (657.14 mg, 3.43 mmol, 2 eq), HOAt (116.64 mg, 856.98 umol, 119.88 uL, 0.5 eq), and NMM (866.81 mg, 8.57 mmol, 942.19 uL, 5 eq) in DCM (5 mL) was added Intermediate 1-6 (500 mg, 1.71 mmol, 1 eq, HCl salt). Then the mixture was stirred at 25° C. for 1 hr. The reaction mixture was poured into water (20 mL) and extracted with DCM (20 mL*2), the organic layer was washed with brine (20 mL) and dried over Na₂SO₄, then concentrated to give the crude product. The crude product was purified by flash silica gel column chromatography (SiO₂, PE:EA=10:1 to 3:1) to give Intermediate 1-8 (590 mg, 1.68 mmol, 98.04% yield, 99.5% purity) as a colorless oil. LCMS (Method E): Rt=0.526 min, [M+H]⁺=320.2.

### Step 6: Synthesis of Intermediate 1-9

To a solution of Intermediate 1-8 (570 mg, 1.63 mmol, 1 eq) in MeOH (3 mL) and H₂O (3 mL) was added LiOH·H₂O (342.32 mg, 8.16 mmol, 5 eq). Then the mixture was stirred at 25° C. for 1 hr. The reaction mixture was poured into water (20 mL) and adjusted to pH=2 with 1N aq. HCl, and it was extracted with EA (20 mL*2), the organic layer was washed with brine (20 mL) and dried over Na₂SO₄. The organics were concentrated to give the crude product. The crude product was purified by SFC separation (column: DAICEL CHIRALPAK IC (250 mm*30 mm, 10 um); mobile phase: [CO₂-EtOH (0.1% NH₃H₂O)]; B %: 42%, isocratic elution mode) to give Intermediate 1-9 (400 mg, 1.16 mmol, 70.99% yield, 97.1% purity) as an off-white solid. LCMS: Rt=0.491 min, [M+H]⁺=336.1. ¹H NMR (400 MHz, DMSO-d6) δ=8.11-7.98 (m, 1H), 7.88-7.80 (m, 1H), 7.39-7.29 (m, 1H), 4.79-4.69 (m, 1H), 4.36 (br d, J=14.0 Hz, 1H), 3.86-3.73 (m, 1H), 3.18 (br s, 1H), 2.74-2.32 (m, 3H), 2.29-2.21 (m, 3H), 2.18-2.15 (m, 3H), 2.05-1.91 (m, 1H), 1.83-1.65 (m, 1H).

### Step 7: Synthesis of Intermediate 1-10

Intermediate 1-9 (400 mg, 1.19 mmol, 1 eq) was purified by SFC (column: DAICEL CHIRALCEL OJ (250 mm*30 mm, 10 um); mobile phase: [CO₂-MeOH (0.1% NH₃H₂O)]; B %: 12%, isocratic elution mode) to give Intermediate 1-10 (150 mg, 433.88 umol, 36.37% yield, 97.0% purity) as a white solid. LCMS (Method E): Rt=0.471 min, [M+H]⁺=336.2. SFC: Rt=0.908 min, ee value >99%.

### Step 8: Synthesis of I-982

To a solution of Intermediate 1-11 (171.39 mg, 223.65 umol, 1 eq, HCl salt), EDCI (85.75 mg, 447.30 umol, 2 eq), HOAt (15.22 mg, 111.83 umol, 15.64 uL, 0.5 eq), and NMM (113.11 mg, 1.12 mmol, 122.94 uL, 5 eq) in DCM (1 mL) was added Intermediate 1-10 (75 mg, 223.65 umol, 1 eq). Then the mixture was stirred at 25° C. for 1 hr. The reaction mixture was poured into water (20 mL) and extracted with DCM (20 mL*2), the organic layer was washed with brine (20 mL) and dried over Na₂SO₄, then concentrated to give the crude product. The crude product was purified by prep-HPLC (column: Waters Xbridge 150*25 mm*5 um; mobile phase: [water (NH₄HCO₃)-ACN]; gradient: 35%-65% B over 15 min). The eluent was lyophilized to give I-982 (20.03 mg, 18.68 umol, 8.35% yield, 97.67% purity) as a white solid. LCMS (Method E): Rt=0.516 min, [M+H]⁺=1047.6. ¹H NMR (400 MHz, DMSO-d6) δ=12.60 (s, 1H), 8.69-8.45 (m, 1H), 8.27 (d, J=8.0 Hz, 1H), 8.05-7.79 (m, 3H), 7.67-7.48 (m, 2H), 7.48-7.20 (m, 4H), 4.88-4.72 (m, 1H), 4.62-4.19 (m, 4H), 4.05-3.80 (m, 2H), 3.71-3.57 (m, 4H), 3.54-3.36 (m, 5H), 3.25-2.99 (m, 6H), 2.67 (br d, J=2.0 Hz, 3H), 2.48-2.26 (m, 2H), 2.17-2.01 (m, 9H), 1.88-1.56 (m, 12H), 1.47-0.99 (m, 9H). SFC: Rt=2.562 min, ee value >99%.

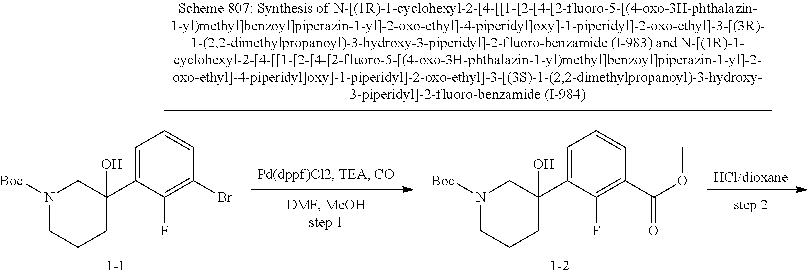 [SMILES: CC(C)(C)OC(=O)N1CCCC(O)(c2cccc(Br)c2F)C1 ; "Pd(dppf)Cl2, TEA, CO" ; COC(=O)c1cccc(C2(O)CCCN(C(=O)OC(C)(C)C)C2)c1F ; "HCl/dioxane"]

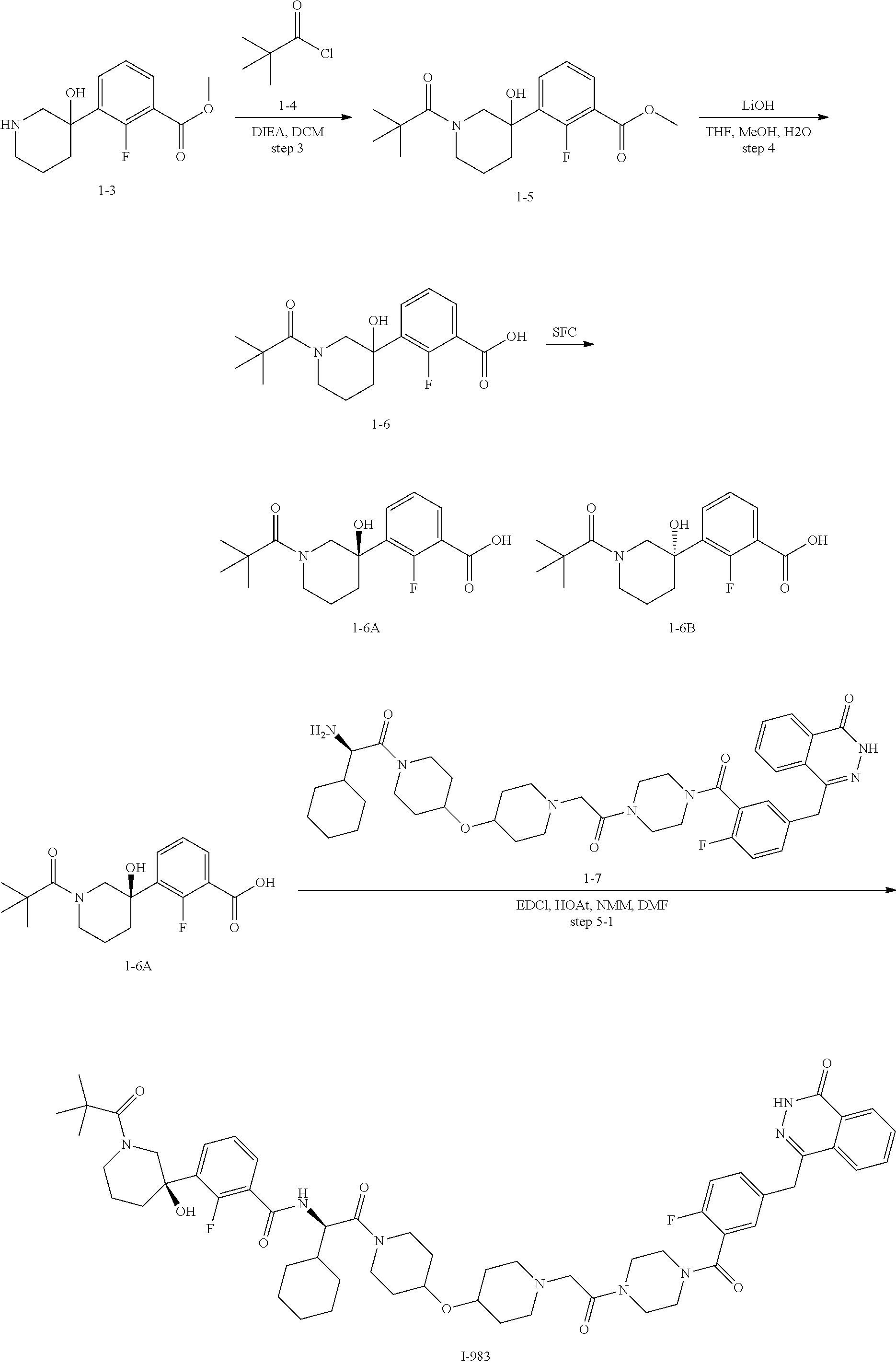

### Step 1: Synthesis of Intermediate 1-2

Solution 1 (P1): Intermediate 1-1 (1 eq, 1 g), Pd(dppf)Cl₂·CH₂Cl₂ (0.05 eq, 0.109 g) and TEA (3 eq, 0.811 g) in DMF (10 mL) and MeOH (10 mL). Solution 2 (P2): DMF:MeOH=1:1. Gas (P3): CO. The volume of flow reactor 1 was 40.25 mL (160° C.). The cooling coils were 3.175(⅛″) mm, 5.013 mL, 25° C. The residence time of the flow reactor 1 was 100 min. The pressure was set to 3.5 MPa. Pump 1 was used to pump solution one at a rate of 0.403 mL/min. Pump 2 was used to pump solution two at a rate of 0.1 mL/min. The CO flow rate 20 mL/min and the pressure was 4.0 MPa. The reaction mixture was collected after running 40 mins. The mixture was purified by flash silica gel chromatography (ISCO; 40 g SepaFlash Silica Flash Column, Eluent of 0˜100% Ethyl acetate/Petroleum ether gradient @40 mL/min). The eluent was concentrated to afford Intermediate 1-2 (500 mg, 1.41 mmol, 50.00% yield) as a yellow oil. LCMS (Method E): Rt=0.948 min, [M+Na]⁺=376.1. ¹H NMR (400 MHz, DMSO-d6) δ=7.91-7.74 (m, 2H), 7.35-7.28 (m, 1H), 3.85 (s, 3H), 3.76-3.45 (m, 2H), 3.37-3.34 (m, 2H), 2.24-2.12 (m, 1H), 1.88-1.68 (m, 2H), 1.46-1.31 (m, 11H).

### Step 2: Synthesis of Intermediate 1-3

To a solution of intermediate 1-2 (500 mg, 1.41 mmol, 1 eq) was added HCl/dioxane (5 mL, 2M). The mixture was stirred at 25° C. for 1 hr. The reaction mixture was concentrated under reduced pressure to give Intermediate 1-3 (400 mg, 1.28 mmol, 90.75% yield, 93% purity, HCl salt) as a white solid. LCMS (Method E): Rt=0.348 min, [M+H]⁺=254.1.

### Step 3: Synthesis of Intermediate 1-5

To a solution of Intermediate 1-3 (300 mg, 1.04 mmol, 1 eq, HCl salt) in DCM (3 mL) was added TEA (209.55 mg, 2.07 mmol, 288.24 μL, 2 eq) and Intermediate 1-4 (137.34 mg, 1.14 mmol, 140.14 μL, 1.1 eq). The mixture was stirred at 20° C. for 12 hr. The reaction mixture was extracted with DCM (3 mL*3). The combined organic layers were washed with brine (5 mL*2), dried over Na₂SO₄, filtered and concentrated under reduced pressure to give a residue. The residue was purified by flash silica gel chromatography (ISCO; 20 g SepaFlash Silica Flash Column, Eluent of 0˜50% Ethyl acetate/Petroleum ether gradient @20 mL/min). The eluent was concentrated to afford Intermediate 1-5 (280 mg, 780.12 μmol, 75.34% yield, 94% purity) as a white solid. LCMS (Method E): Rt=0.507 min, [M-OH]⁺=320.1.

### Step 4: Synthesis of Intermediate 1-6A and Intermediate 1-6B

To a solution of Intermediate 1-5 (280 mg, 829.91 μmol, 1 eq) in THF (2 mL) was added LiOH·H₂O (69.65 mg, 1.66 mmol, 2 eq) in H₂O (2 mL) and MeOH (2 mL). The mixture was stirred at 25° C. for 1 hr. The reaction was acidified with 0.5M aq·HCl to pH<7, and extracted with EtOAc (3 mL*3). The combined organic layers were dried over Na₂SO₄, filtered and concentrated under reduced pressure to give product (280 mg). 50 mg of the product was used for the next step without further purification. Then the remainder (230 mg) was separated by SFC (column: DAICEL CHIRALPAK IC (250 mm*30 mm, 10 um); mobile phase: [CO₂-ACN/i-PrOH (0.1% NH₃H₂O)]; B %: 30%, isocratic elution mode) to give Intermediate 1-6A (Rt=1.366 min) and Intermediate 1-6B (Rt=1.634 min). Intermediate 1-6A (100 mg, 309.25 μmol, 37.26% yield) was obtained as a white solid. LCMS: Rt=0.436 min, [M+H]⁺=324.0. Intermediate 1-6B (100 mg, 309.25 μmol, 37.26% yield) was obtained as a white solid. LCMS: Rt=0.442 min, [M+H]⁺=324.0.

### Step 5-1: Synthesis of I-983

To a solution of intermediate 1-6A (30 mg, 92.78 μmol, 1 eq) and intermediate 1-7 (78.21 mg, 102.05 μmol, 1.1 eq, HCl salt) in DMF (1 mL) was added EDCI (53.36 mg, 278.33 μmol, 3 eq), NMM (75.07 mg, 742.21 μmol, 81.60 μL, 8 eq) and HOAt (12.63 mg, 92.78 μmol, 12.98 μL, 1 eq). The mixture was stirred at 25° C. for 1 hr. The mixture was concentrated under reduced pressure to give a residue. The residue was purified by prep-HPLC (column: Phenomenex luna C18 150*25 mm*10 um; mobile phase: [water (FA)-ACN]; gradient: 23%-53% B over 10 min). The eluent was lyophilized to afford I-983 (36 mg, 33.29 μmol, 35.89% yield, FA salt) as a white solid. LCMS (Method E): Rt=0.494 min, [M+H]⁺=1035.6. SFC: Rt=1.840 min. ¹H NMR (400 MHz, DMSO-d6) δ=12.59 (s, 1H), 8.51-8.37 (m, 1H), 8.22 (s, 1H), 8.14 (s, 1H), 7.97 (br d, J=8.0 Hz, 1H), 7.93-7.80 (m, 2H), 7.75-7.70 (m, 1H), 7.47-7.34 (m, 3H), 7.28-7.19 (m, 2H), 5.24 (s, 1H), 4.88-4.69 (m, 1H), 4.40-4.30 (m, 2H), 4.28-4.18 (m, 1H), 4.05 (br d, J=13.2 Hz, 1H), 4.02-3.83 (m, 2H), 3.68-3.55 (m, 4H), 3.53-3.43 (m, 3H), 3.36 (br s, 2H), 3.30-3.24 (m, 2H), 3.20-2.87 (m, 6H), 2.77-2.63 (m, 2H), 2.38-2.01 (m, 4H), 1.91-1.86 (m, 1H), 1.86-1.67 (m, 9H), 1.60 (br d, J=7.6 Hz, 2H), 1.50 (br d, J=12.8 Hz, 1H), 1.47-1.34 (m, 3H), 1.20 (s, 9H), 1.16-1.00 (m, 4H). ¹⁹F NMR (377 MHz, DMSO-d6) δ=−113.31 (br d, J=24.7 Hz, 1F), −119.77 (br s, 1F).

### Step 5-2: Synthesis of I-984

To a solution of intermediate 1-6B (30 mg, 92.78 μmol, 1 eq) and intermediate 1-7 (78.21 mg, 102.05 μmol, 1.1 eq, HCl salt) in DMF (1 mL) was added EDCI (53.36 mg, 278.33 μmol, 3 eq), NMM (75.07 mg, 742.21 μmol, 81.60 μL, 8 eq) and HOAt (12.63 mg, 92.78 μmol, 12.98 μL, 1 eq). The mixture was stirred at 25° C. for 1 hr. The mixture was concentrated under reduced pressure to give a residue. The residue was purified by prep-HPLC (column: Phenomenex luna C18 150*25 mm*10 um; mobile phase: [water (FA)-ACN]; gradient: 20%-50% B over 10 min). The eluent was lyophilized to afford I-984 (36 mg, 33.29 μmol, 35.89% yield, FA salt) as a white solid. LCMS (Method E): Rt=0.496 min, [M+H]⁺=1035.6. SFC: Rt=1.473 min. ¹H NMR (400 MHz, DMSO-d6) δ=12.59 (s, 1H), 8.51-8.35 (m, 1H), 8.33-8.23 (m, 1H), 8.17-8.14 (m, 1H), 7.97 (br d, J=8.0 Hz, 1H), 7.92-7.79 (m, 2H), 7.75-7.68 (m, 1H), 7.49-7.34 (m, 3H), 7.28-7.19 (m, 2H), 5.32-5.15 (m, 1H), 4.82-4.74 (m, 1H), 4.33 (s, 2H), 4.29-4.21 (m, 1H), 4.09 (br d, J=13.2 Hz, 1H), 4.03-3.82 (m, 2H), 3.69-3.56 (m, 4H), 3.53-3.43 (m, 3H), 3.38-3.35 (m, 2H), 3.29-3.24 (m, 2H), 3.15 (br s, 6H), 2.73-2.59 (m, 2H), 2.40-1.96 (m, 4H), 1.91-1.85 (m, 1H), 1.84-1.65 (m, 9H), 1.59 (br d, J=11.2 Hz, 2H), 1.54-1.48 (m, 1H), 1.46-1.33 (m, 3H), 1.20 (s, 9H), 1.16-0.99 (m, 4H). ¹⁹F NMR (377 MHz, DMSO-d6) δ=−113.31 (br s, 1F), −119.77 (br s, 1F).

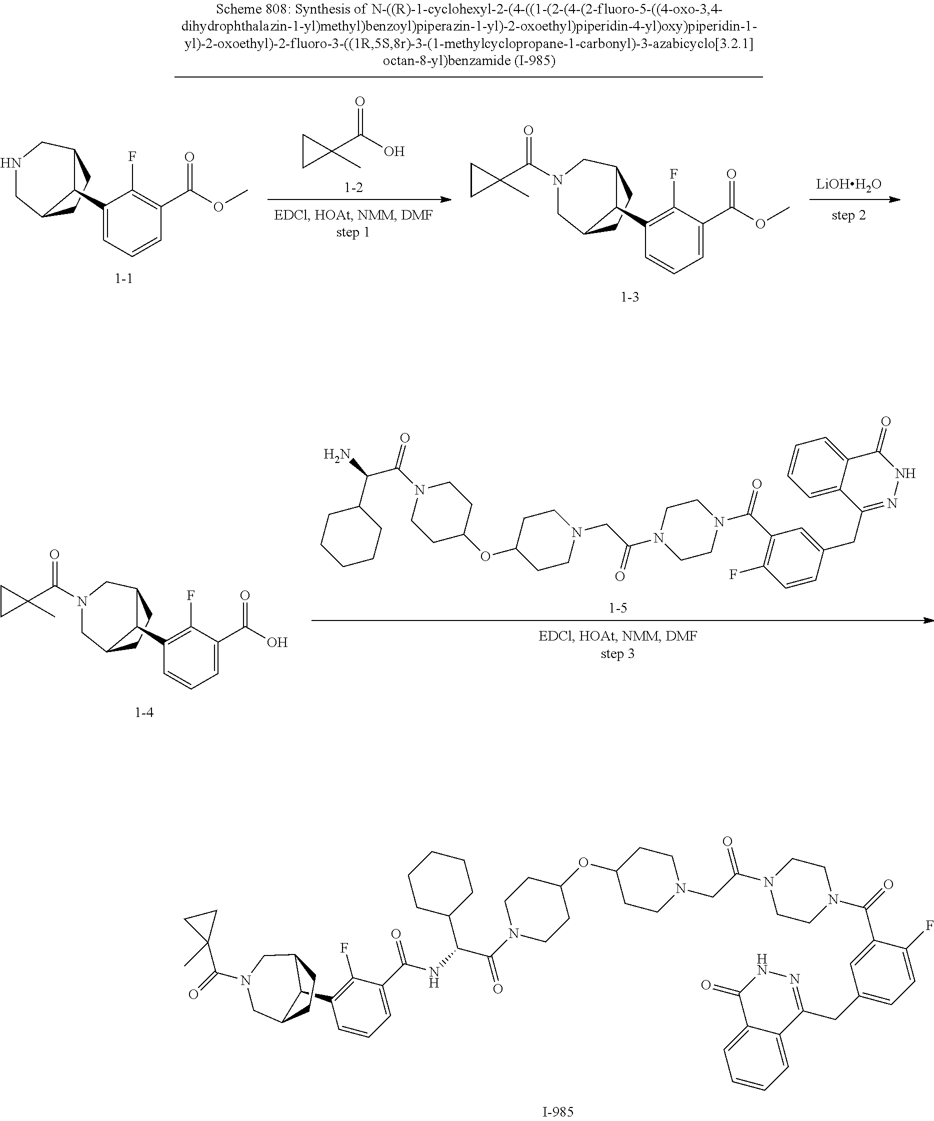

### Step 1: Synthesis of Intermediate 1-3

To a solution of Intermediate 1-2 (82.29 mg, 733.90 umol, 1.1 eq), EDCI (255.80 mg, 1.33 mmol, 2 eq), HOAt (45.41 mg, 333.59 umol, 46.67 uL, 0.5 eq), and NMM (337.42 mg, 3.34 mmol, 366.76 uL, 5 eq) in DCM (5 mL) was added Intermediate 1-1 (200 mg, 667.18 umol, 1 eq, HCl salt). Then the mixture was stirred at 25° C. for 1 hr. The reaction mixture was poured into water (20 mL) and extracted with DCM (20 mL*2), the organic layer was washed with brine (20 mL) and dried over Na₂SO₄, then concentrated to give the crude product. The crude product was purified by flash silica gel column chromatography (SiO₂, PE:EA=10:1 to 3:1) to give Intermediate 1-3 (230 mg, 636.43 umol, 95.39% yield, 98.9% purity) as colorless oil. LCMS (Method E): Rt=0.599 min, [M+H]⁺=346.1.

### Step 2: Synthesis of Intermediate 1-4

To a solution of Intermediate 1-3 (220 mg, 636.43 umol, 95.39% yield, 98.9% purity) in MeOH (3 mL) and H₂O (3 mL) was added LiOH·H₂O (129.15 mg, 3.08 mmol, 5 eq). Then the mixture was stirred at 25° C. for 1 hr. The reaction mixture was poured into water (20 mL) and adjusted to pH=2 with 1N aq·HCl, and extracted with EA (20 mL*2). The organic layer was washed with brine (20 mL) and dried over Na₂SO₄, then concentrated to give Intermediate 1-4 (150 mg, 435.07 umol, 70.68% yield, 100% purity) as a white solid. LCMS (Method E): Rt=0.461 min, [M+H]⁺=332.1.

### Step 3: Synthesis of I-985

To a solution of Intermediate 1-5 (167.38 mg, 218.65 umol, 1 eq, HCl salt), EDCI (85.75 mg, 447.30 umol, 2 eq), HOAt (15.22 mg, 111.83 umol, 0.5 eq), and NMM (113.11 mg, 1.12 mmol, 5 eq) in DCM (1 mL) was added Intermediate 1-4 (75 mg, 218.65 umol, 1 eq). Then the mixture was stirred at 25° C. for 1 hr. The reaction mixture was poured into water (20 mL) and extracted with DCM (20 mL*2), the organic layer was washed with brine (20 mL) and dried over Na₂SO₄, then concentrated to give the crude product. The crude product was purified by prep-HPLC (column: Waters Xbridge 150*25 mm*5 um; mobile phase: [water (NH₄HCO₃)-ACN]; gradient: 18%-48% B over 15 min). The eluent was lyophilized to give I-985 (44.55 mg, 40.82 umol, 18.04% yield, 99.81% purity, FA salt) as a white solid. LCMS (Method E): Rt=0.522 min, [M+H]⁺=1055.6. ¹H NMR (400 MHz, DMSO-d6) δ=12.60 (s, 1H), 8.45-8.31 (m, 1H), 8.27 (br d, J=8.0 Hz, 1H), 8.17 (s, 1H), 8.03-7.80 (m, 3H), 7.51-7.31 (m, 4H), 7.29-7.15 (m, 2H), 4.86-4.75 (m, 1H), 4.34 (s, 2H), 4.17 (br d, J=12.0 Hz, 2H), 4.05-3.77 (m, 3H), 3.64 (br s, 5H), 3.54-3.20 (m, 7H), 3.15-3.00 (m, 3H), 2.68 (br s, 2H), 2.54 (br s, 2H), 2.23-2.06 (m, 2H), 1.89-1.56 (m, 12H), 1.42 (br d, J=3.2 Hz, 5H), 1.32-0.94 (m, 9H), 0.82 (br s, 2H), 0.57 (br s, 2H).

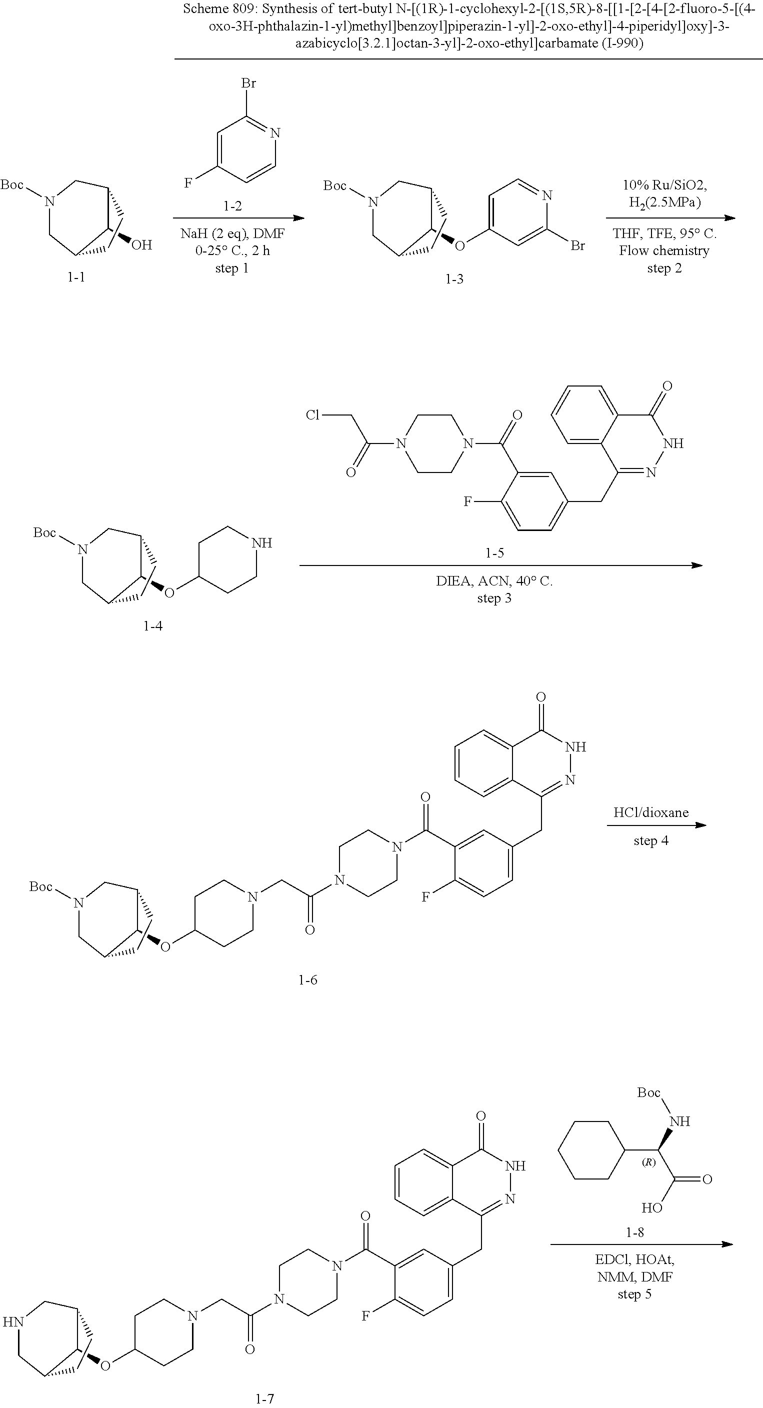

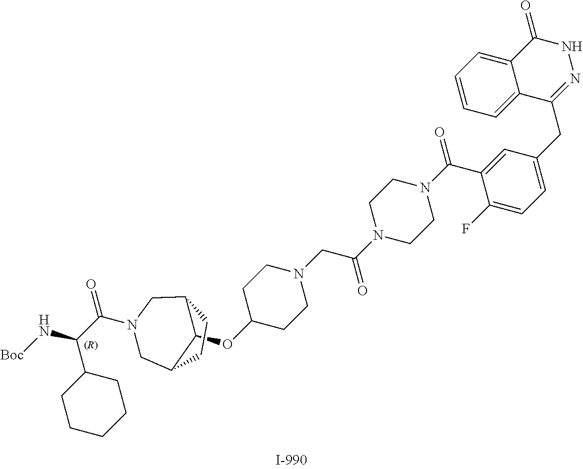

### Step 1: Synthesis of Intermediate 1-3

To a solution of Intermediate 1-1 (5 g, 22.00 mmol, 1 eq) in DMF (50 mL) was added NaH (1.76 g, 43.99 mmol, 60% purity, 2 eq) under N₂ atmosphere. The mixture was stirred at 0° C. for 0.5 h, then Intermediate 1-2 (5.81 g, 33.00 mmol, 1.5 eq) was added to the reaction mixture and the mixture was stirred at 25° C. for 1.5 h under N₂. The reaction mixture was quenched with a saturated NH₄Cl solution and extracted with EA (50 mL*3). The combined organic layers were washed with brine (50 mL*3), dried over anhydrous Na₂SO₄, filtered and concentrated under reduced pressure to give a residue. The residue was triturated with methyl tert-butyl ether (50 mL) for 20 min, then filtered off to collect the white solid, and the white solid was dried under vacuum to give Intermediate 1-3 (6.4 g, 16.53 mmol, 75.15% yield, 99% purity) as a white solid. LCMS (Method E): Rt=0.599 min, [M+H]⁺=385.2. ¹HNMR (400 MHz, DMSO-d6) δ=8.19 (d, J=6.0 Hz, 1H), 7.38 (d, J=2.0 Hz, 1H), 7.16-7.14 (m, 1H), 4.73-4.71 (m, 1H), 3.65-3.47 (m, 2H), 3.18 (d, J=12.8 Hz, 1H), 3.04 (d, J=12.4 Hz, 1H), 2.37-2.25 (m, 2H), 1.84-1.72 (m, 2H), 1.51-1.44 (m, 2H), 1.39 (s, 9H). SFC: Rt=1.516 min, ee value>99%.

### Step 2: Synthesis of Intermediate 1-4

Solution 1: Intermediate 1-3 (1 eq, 6.3 g) and TEA (2 eq, 3.327 g) in THF (63 mL) and TFE (63 mL). The fixed bed (volume: 20 mL) was completely packed with granular catalyst 10% Ru/SiO₂ (1.00 eq, 10 g). The H₂ back pressure regulator was adjusted to 2.5 MPa, and the flow rate of H₂ was 60 mL/min. Then Solution 1 was pumped (1.212 mL/min) to the fixed bed (9.525(½″) mm, 4 mL, 95° C.). The reaction mixture was collected from the reactor output. The mixture was concentrated to give Intermediate 1-4 (4.5 g, crude) as a white solid. ¹HNMR (400 MHz, DMSO-d6) δ=8.75-8.23 (m, 2H), 3.77-3.66 (m, 2H), 3.57-3.41 (m, 2H), 3.24-3.13 (m, 3H), 3.12-3.04 (m, 2H), 3.03-2.92 (m, 2H), 2.10-1.90 (m, 3H), 1.77-1.52 (m, 4H), 1.38 (s, 9H), 1.18 (m, 2H).

### Step 4: Synthesis of Intermediate 1-6

To a mixture of Intermediate 1-4 (250.00 mg, 805.33 μmol, 1 eq) in CH₃CN (4 mL) was added Intermediate 1-5 (356.66 mg, 805.33 μmol, 1 eq) and DIPEA (312.25 mg, 2.42 mmol, 420.82 μL, 3 eq) at 20° C. The mixture was stirred at 40° C. for 12 hrs. The reaction mixture was poured into water (10 mL), then extracted with EA (10 mL*3), the organic layer was washed with brine (10 mL), dried over Na₂SO₄, filtered and concentrated to give the crude product. The crude product was purified by reversed phase chromatography (FA condition), and the eluent was concentrated to remove organic solvents. The residual aqueous solution was extracted with DCM (50 mL*3), the organic layer was washed with brine (100 mL), dried over Na₂SO₄, filtered and concentrated to give Intermediate 1-6 (330 mg, 437.34 μmol, 54.31% yield, 95% purity) as a white solid. LCMS (Method E): Retention time: 0.470 min, [M+H]⁺=717.3.

### Step 4: Synthesis of Intermediate 1-7

A mixture of Intermediate 1-6 (330.00 mg, 460.35 μmol, 1 eq) in HCl/dioxane (5 mL, 2M) was stirred at 20° C. for 1 hr. the mixture was concentrated to give Intermediate 1-7 (300 mg, 445.51 mol, 96.78% yield, 97% purity, HCl salt) as a white solid. LCMS (Method E): Retention time: 0.368 min, [M+H]⁺=617.3. SFC: Rt=1.436 min, ee value=97.4%.

### Step 5: Synthesis of I-990

To a mixture of Intermediate 1-8 (118.19 mg, 459.29 μmol, 1 eq) in DMF (3 mL) was added EDCI (264.14 mg, 1.38 mmol, 3 eq), HOAt (62.51 mg, 459.29 μmol, 64.25 μL, 1 eq), NMM (371.64 mg, 3.67 mmol, 403.96 μL, 8 eq) and Intermediate 1-7 (300 mg, 459.29 μmol, 1 eq, HCl salt). The mixture was stirred at 20° C. for 2 hrs. The reaction mixture was poured into water (10 mL), then extracted with EA (10 mL*3), the organic layer was washed with brine (10 mL), dried over Na₂SO₄, filtered and concentrated to give the crude product. The crude product was purified by Prep-HPLC (column: Phenomenex luna C18 150*25 mm*10 um; mobile phase: [water (FA)-ACN]; gradient: 28%-588% B over 11 min), and the eluent was concentrated to remove organic solvents. The residual aqueous solution was lyophilized to give I-990 (189.12 mg, 219.29 μmol, 47.75% yield, 99.26% purity) as a white solid. LCMS (Method E): Retention time: 0.497 min, [M+H]⁺=856.4. ¹HNMR (400 MHz, DMSO-d6) δ=8.25 (m, 1H), 8.16 (s, 1H), 7.98-7.92 (m, 1H), 7.91-7.79 (m, 2H), 7.46-7.39 (m, 1H), 7.35 (m, 1H), 7.22 (t, J=8.9 Hz, 1H), 6.78-6.61 (m, 1H), 4.32 (s, 2H), 4.21 (br s, 1H), 3.97 (m, 1H), 3.82-3.69 (m, 2H), 3.68-3.62 (m, 2H), 3.52-3.32 (m, 6H), 3.27-3.07 (m, 4H), 2.94 (m, 1H), 2.78-2.62 (m, 2H), 2.30-2.12 (m, 2H), 2.11-1.97 (m, 2H), 1.84 (br s, 2H), 1.73-1.48 (m, 9H), 1.45-1.28 (m, 11H), 1.26-1.19 (m, 1H), 1.17-1.03 (m, 3H), 1.01-0.79 (m, 2H). ¹⁹F NMR (377 MHz, DMSO-d6) δ=−119.72 (br s, 1F). SFC: Rt=1.505 min, devalue >99%.

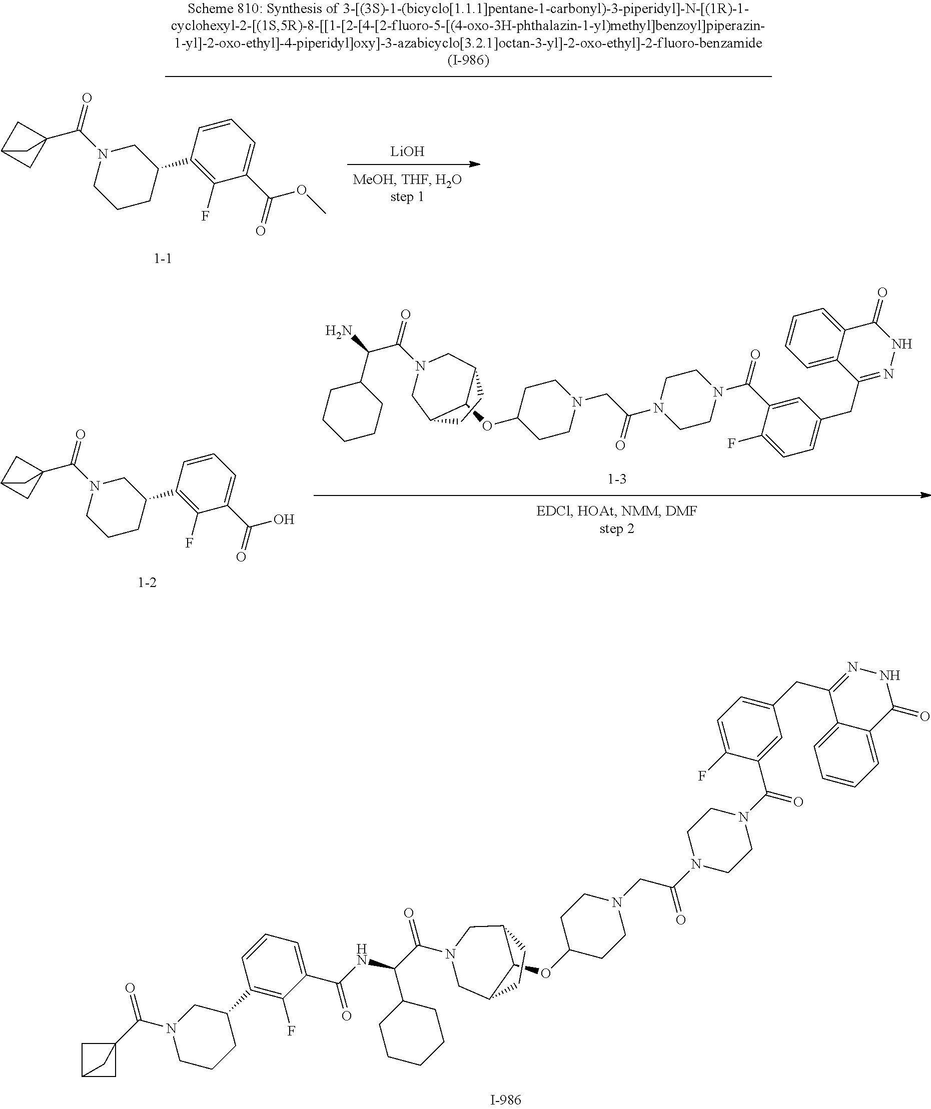 [SMILES: COC(=O)c1cccc([C@@H]2CCCN(C(=O)C34CC(C3)C4)C2)c1F ; "LiOH" ; O=C(O)c1cccc([C@@H]2CCCN(C(=O)C34CC(C3)C4)C2)c1F ; N[C@@H](C(=O)N1C[C@H]2CC[C@@H](C1)[C@H]2OC1CCN(CC(=O)N2CCN(C(=O)c3cc(Cc4n[nH]c(=O)c5ccccc45)ccc3F)CC2)CC1)C1CCCCC1 ; O=C(N[C@@H](C(=O)N1C[C@H]2CC[C@@H](C1)[C@H]2OC1CCN(CC(=O)N2CCN(C(=O)c3cc(Cc4n[nH]c(=O)c5ccccc45)ccc3F)CC2)CC1)C1CCCCC1)c1cccc([C@@H]2CCCN(C(=O)C34CC(C3)C4)C2)c1F]

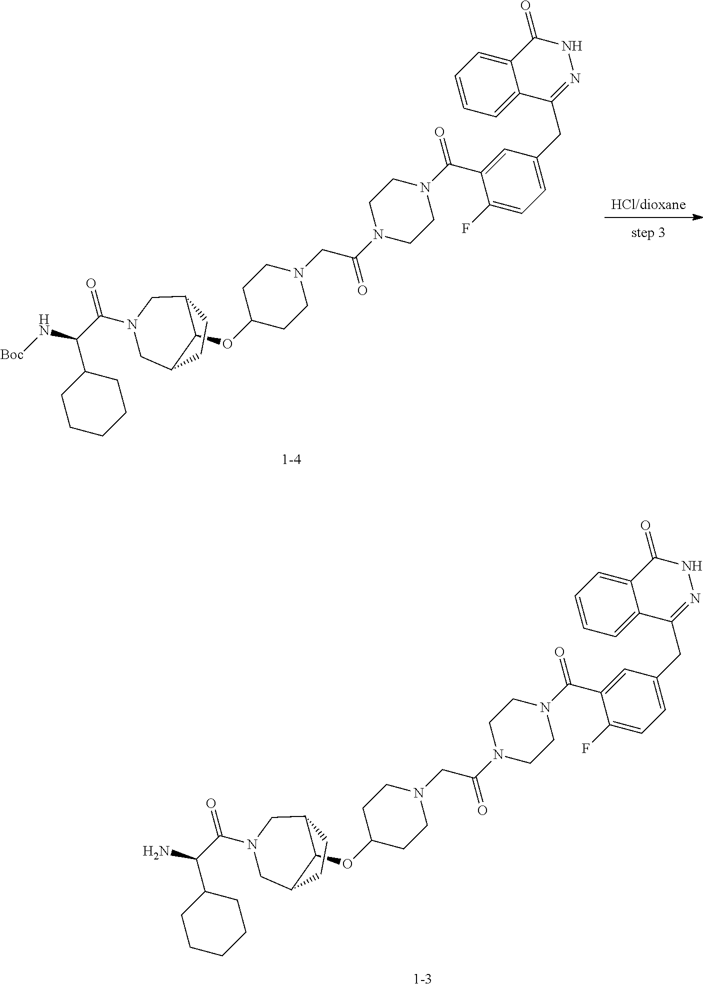 [SMILES: CC(C)(C)OC(=O)N[C@@H](C(=O)N1C[C@H]2CC[C@@H](C1)[C@H]2OC1CCN(CC(=O)N2CCN(C(=O)c3cc(Cc4n[nH]c(=O)c5ccccc45)ccc3F)CC2)CC1)C1CCCCC1 ; "HCl/dioxane" ; N[C@@H](C(=O)N1C[C@H]2CC[C@@H](C1)[C@H]2OC1CCN(CC(=O)N2CCN(C(=O)c3cc(Cc4n[nH]c(=O)c5ccccc45)ccc3F)CC2)CC1)C1CCCCC1]

### Step 1: Synthesis of Intermediate 1-2

To a solution of Intermediate 1-1 (1.3 g, 3.92 mmol, 1 eq) in MeOH (5 mL), THF (5 mL) and H₂O (5 mL) was added LiOH·H₂O (493.83 mg, 11.77 mmol, 3 eq), and the mixture was stirred at 25° C. for 1 hr. The pH of the mixture was adjusted to 6 with citric acid (sat.), and extracted with DCM (20 mL*2). The combined organic layers were washed with brine (20 mL), dried over Na₂SO₄, filtered and concentrated under reduced pressure to give Intermediate 1-2 (1.05 g, 3.27 mmol, 83.41% yield, 98.9% purity) as a white solid. LCMS (Method E): Rt=0.477 min, [M+H]⁺=318.3. ¹HNMR (400 MHz, CHLOROFORM-d) δ=8.04-7.80 (m, 1H), 7.57-7.38 (m, 1H), 7.28-7.15 (m, 1H), 4.74-4.62 (m, 1H), 4.42-4.19 (m, 1H), 3.18-2.98 (m, 2H), 2.87-2.56 (m, 1H), 2.55-2.44 (m, 1H), 2.32-2.12 (m, 6H), 2.11-1.99 (m, 1H), 1.94-1.74 (m, 2H), 1.73-1.56 (m, 1H). SFC: Rt=1.639 min, de value >99%.

### Step 2: Synthesis of I-986

To a mixture of Intermediate 1-2 (100 mg, 315.11 μmol, 1 eq) in DMF (3 mL) was added EDCI (181.22 mg, 945.32 μmol, 3 eq), HOAt (42.89 mg, 315.11 μmol, 44.08 μL, 1 eq), NMM (159.36 mg, 1.58 mmol, 173.22 μL, 5 eq) and Intermediate 1-3 (249.68 mg, 315.11 μmol, 1 eq, HCl salt), and the mixture was stirred at 20° C. for 2 hrs. The reaction mixture was combined with another lot of material (50 mg) and poured into water (10 mL), then extracted with EA (10 mL*3), the organic layer was washed with brine (10 mL), dried over Na₂SO₄, filtered and concentrated to give the crude product. The crude product was purified by reversed phase chromatography (Eluent of 33-43% of MeCN/H₂O (0.5% FA), flow rate: 80 mL/min) then the eluent was concentrated to remove organic solvents. The residual aqueous solution was lyophilized to give I-986 (216.49 mg, 199.96 μmol, 63.46% yield, 97.47% purity, FA salt) as a white solid. LCMS (Method E): Retention time: 0.514 min, [M+H]⁺=1055.6. ¹HNMR (400 MHz, METHANOL-d4) δ=8.42 (s, 1H), 8.39-8.32 (m, 1H), 7.98-7.91 (m, 1H), 7.91-7.79 (m, 2H), 7.59-7.45 (m, 3H), 7.41-7.33 (m, 1H), 7.30-7.20 (m, 1H), 7.20-7.11 (m, 1H), 5.04-4.89 (m, 2H), 4.53 (m, 1H), 4.42-4.27 (m, 3H), 4.13 (m, 1H), 3.97-3.42 (m, 13H), 3.28-2.95 (m, 6H), 2.84-2.60 (m, 3H), 2.52-2.39 (m, 1H), 2.28-2.11 (m, 9H), 2.10-1.96 (m, 3H), 1.93-1.55 (m, 14H), 1.50-1.39 (m, 1H), 1.37-1.02 (m, 5H). ¹⁹F NMR (377 MHz, METHANOL-d4) δ=−120.72 (br d, J=20.2 Hz, 1F), −121.35-−121.80 (m, 1F). SFC: Rt=1.847 min, de value >99%.

### Step 3: Synthesis of Intermediate 1-3

A mixture of Intermediate 1-4 (2.60 g, 3.63 mmol, 1 eq) in HCl/dioxane (30 mL, 2M) was stirred at 20° C. for 1 hr. The mixture was concentrated to give Intermediate 1-3 (2.3 g, 3.52 mmol, 97.08% yield, HCl salt) as a white solid. LCMS (method E): Retention time: 0.367 min, [M+H]⁺=617.4.

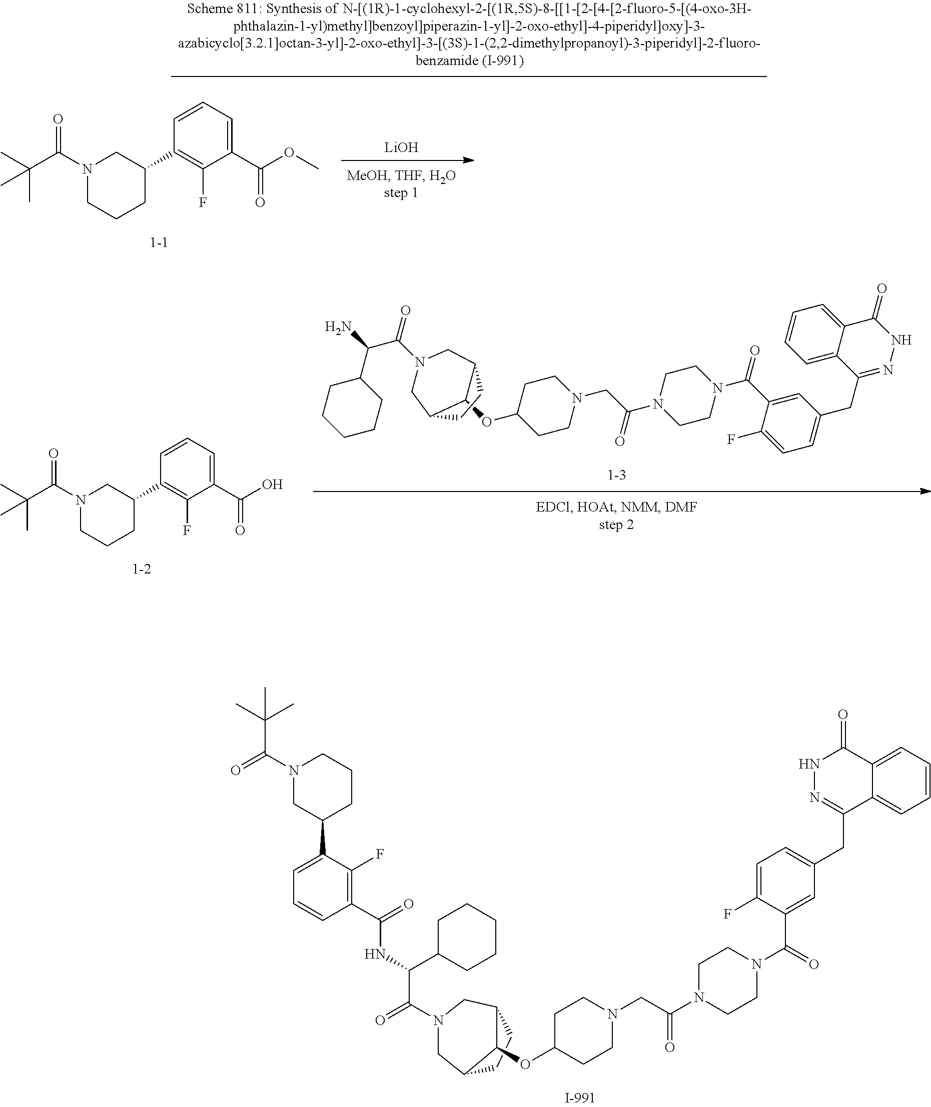 [SMILES: COC(=O)c1cccc([C@@H]2CCCN(C(=O)C(C)(C)C)C2)c1F ; "LiOH" ; CC(C)(C)C(=O)N1CCC[C@@H](c2cccc(C(=O)O)c2F)C1 ; N[C@@H](C(=O)N1C[C@H]2CC[C@@H](C1)[C@H]2OC1CCN(CC(=O)N2CCN(C(=O)c3cc(Cc4n[nH]c(=O)c5ccccc45)ccc3F)CC2)CC1)C1CCCCC1 ; CC(C)(C)C(=O)N1CCC[C@@H](c2cccc(C(=O)N[C@@H](C(=O)N3C[C@H]4CC[C@@H](C3)[C@H]4OC3CCN(CC(=O)N4CCN(C(=O)c5cc(Cc6n[nH]c(=O)c7ccccc67)ccc5F)CC4)CC3)C3CCCCC3)c2F)C1]

### Step 1: Synthesis of Intermediate 1-2

To a solution of Intermediate 1-1 (1.1 g, 3.42 mmol, 1 eq) in MeOH (5 mL), THF (5 mL) and H₂O (5 mL) was added LiOH·H₂O (430.88 mg, 10.27 mmol, 3 eq), and the mixture was stirred at 25° C. for 1 hr. The pH of the mixture was adjusted to 6 with citric acid (sat.), and extracted with DCM (20 mL*2). The combined organic layers were washed with brine (20 mL), dried over Na₂SO₄, filtered and concentrated under reduced pressure to give Intermediate 1-2 (0.92 g, 2.98 mmol, 87.02% yield, 99.5% purity) as a white solid. LCMS (Method E): Rt=0.498 min, [M+H]⁺=308.3. ¹HNMR (400 MHz, CHLOROFORM-d) δ=7.98-7.83 (m, 1H), 7.54-7.40 (m, 1H), 7.21 (t, J=7.6 Hz, 1H), 4.65-4.42 (m, 2H), 3.21-3.01 (m, 1H), 3.00-2.89 (m, 1H), 2.88-2.74 (m, 1H), 2.12-2.00 (m, 1H), 1.92-1.73 (m, 2H), 1.72-1.57 (m, 1H), 1.32 (s, 9H). SFC: Rt=0.989 min, ee value >99%.

### Step 3: Synthesis of I-991

To a mixture of Intermediate 1-2 (150 mg, 488.03 μmol, 1 eq) in DMF (3 mL) was added EDCI (280.67 mg, 1.46 mmol, 3 eq), HOAt (66.43 mg, 488.03 μmol, 68.27 μL, 1 eq), NMM (246.81 mg, 2.44 mmol, 268.28 μL, 5 eq) and Intermediate 1-3 (386.70 mg, 488.03 μmol, 1 eq, HCl salt). The mixture was stirred at 20° C. for 2 hrs. The reaction mixture was poured into water (10 mL), then extracted with EA (10 mL*3), the organic layer was washed with brine (10 mL), dried over Na₂SO₄, filtered and concentrated to give the crude product. The crude product was purified by Prep-HPLC (column: Phenomenex luna C18 150*25 mm*10 um; mobile phase: [water (FA)-ACN]; gradient: 33%-63% B over 11 min), then the eluent was concentrated to remove organic solvents, and the residual aqueous solution was lyophilized to give I-991 (86.17 mg, 82.30 μmol, 16.86% yield, 99.83% purity, FA salt) as a white solid. LCMS (Method E): Retention time: 0.523 min, [M+H]⁺=1045.7. SFC: Retention time: 1.742 min, ee value >99%. ¹HNMR (400 MHz, METHANOL-d4) δ=8.52 (s, 1H), 8.42-8.31 (m, 1H), 7.99-7.91 (m, 1H), 7.90-7.79 (m, 2H), 7.60-7.44 (m, 3H), 7.42-7.32 (m, 1H), 7.30-7.21 (m, 1H), 7.16 (m, 1H), 5.08-4.98 (m, 2H), 4.59 (s, 1H), 4.54-4.44 (m, 2H), 4.42-4.35 (m, 2H), 4.12 (m, 1H), 4.00-3.64 (m, 9H), 3.63-3.46 (m, 4H), 3.25-3.17 (m, 1H), 3.13-3.03 (m, 1H), 3.01-2.90 (m, 2H), 2.78 (m, 2H), 2.48-2.31 (m, 2H), 2.26-2.25 (m, 1H), 2.25-2.13 (m, 2H), 2.02-1.58 (m, 18H), 1.50-1.41 (m, 1H), 1.33-1.27 (m, 10H), 1.25-1.15 (m, 2H), 1.15-1.07 (m, 1H). ¹⁹F NMR (377 MHz, METHANOL-d4) δ=−120.74 (br d, J=13.6 Hz, 1F), −121.24-−121.98 (m, 1F).

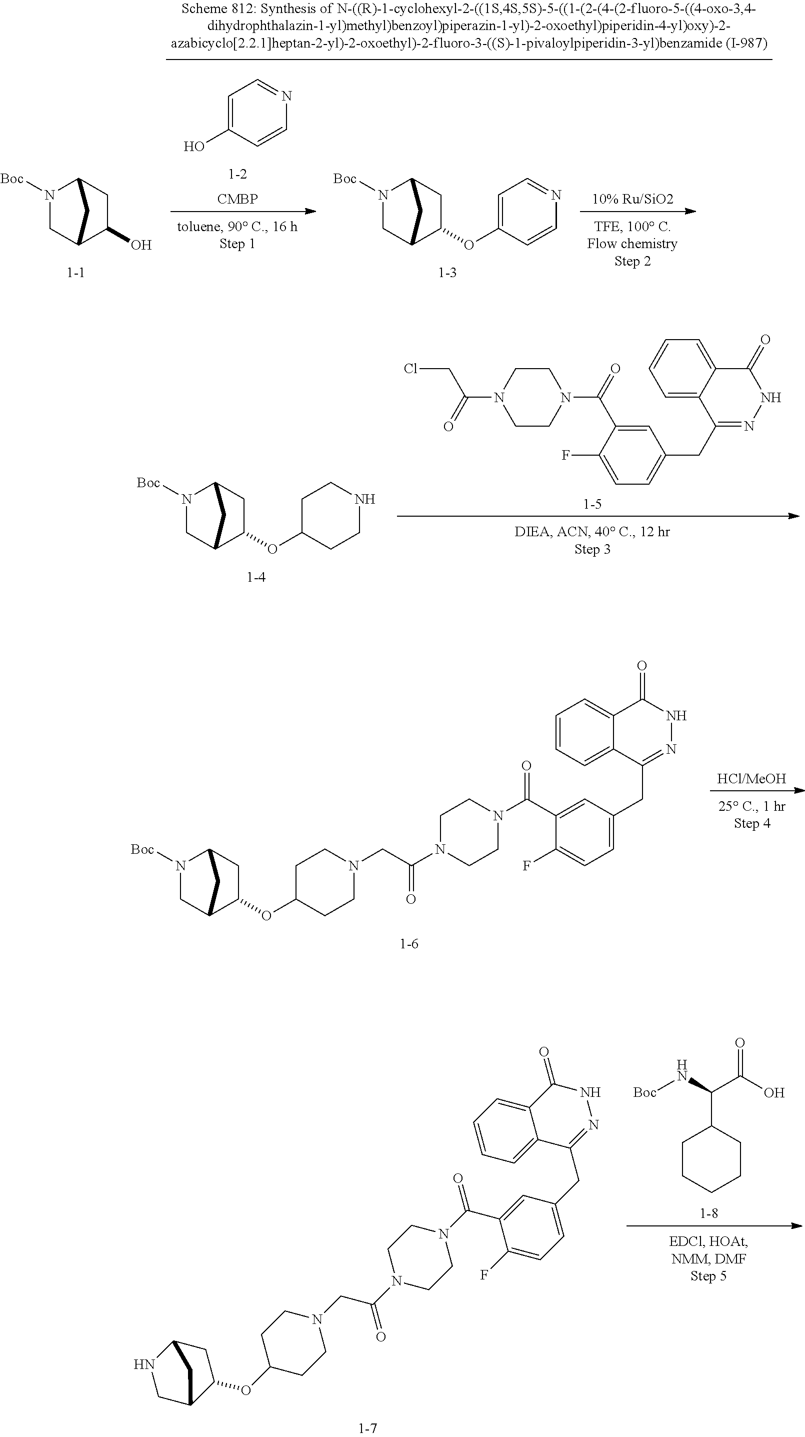

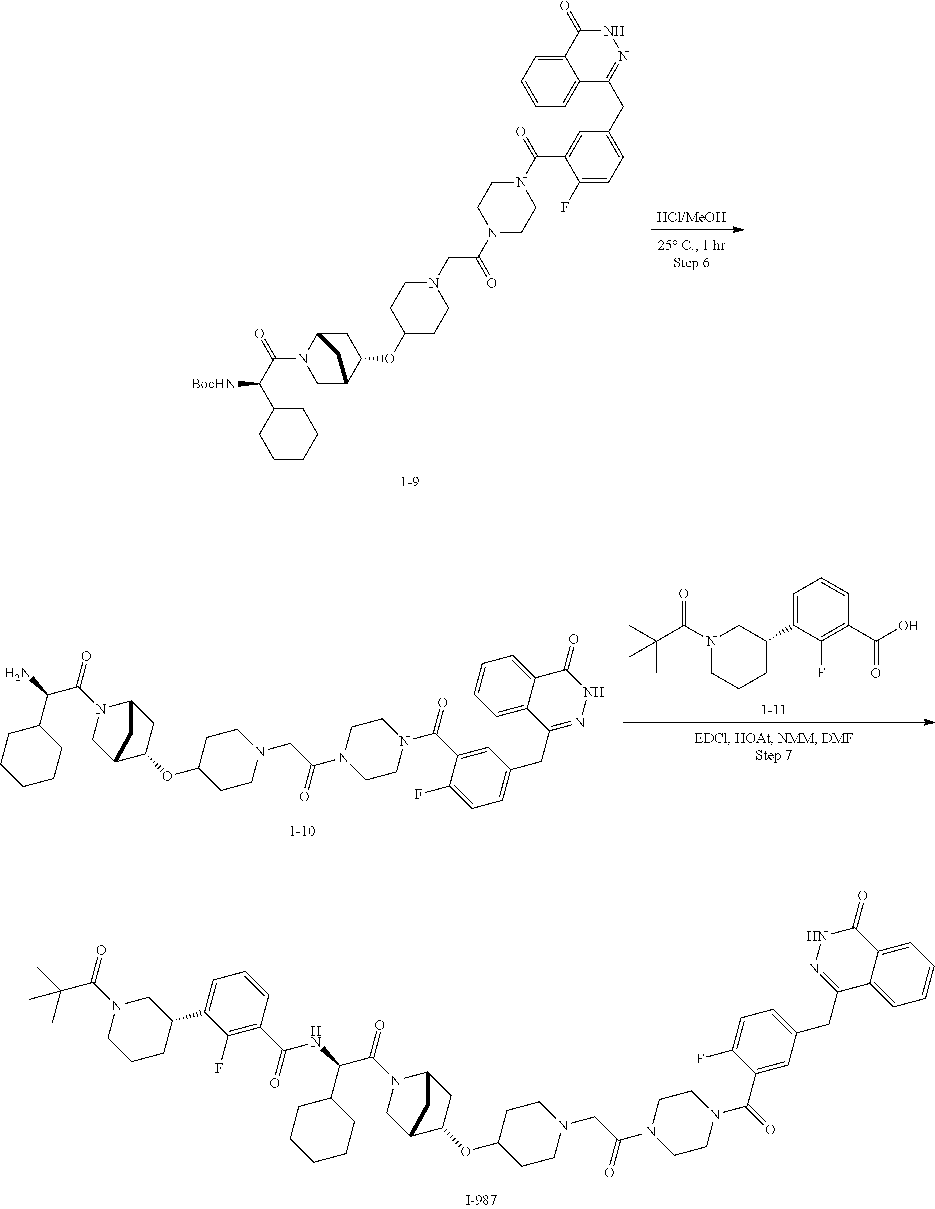

### Step 1: Synthesis of Intermediate 1-3

To a solution of Intermediate 1-1 (2.25 g, 10.55 mmol, 1 eq) in toluene (22 mL) was added Intermediate 1-2 (1.00 g, 10.55 mmol, 1 eq) and CMBP (3.82 g, 15.82 mmol, 1.5 eq), and the mixture was stirred at 90° C. for 16 hr. The mixture diluted with water (100 mL), and extracted with EA (100 mL*2). The combined organic phase was washed with brine (100 mL), dried over Na₂SO₄, filtered and concentrated under reduced pressure to give crude product. The crude product was purified by flash silica gel chromatography (PE:EA=0:1, Rf=0.1, ISCO; 120 g SepaFlash Silica Flash Column, Eluent of 0˜100% EA/PE, gradient @100 mL/min), and the eluent was concentrated in vacuum to give Intermediate 1-3 (0.75 g, 2.58 mmol, 24.48% yield) as a yellow oil. LCMS (Method E): Rt=0.414 min, [M+H]⁺=291.3. ¹H NMR (400 MHz, CHLOROFORM-d) δ=8.42 (br d, J=5.6 Hz, 2H), 6.86-6.69 (m, 2H), 4.91-4.66 (m, 1H), 4.37-4.18 (m, 1H), 3.79-3.58 (m, 1H), 3.31-3.11 (m, 1H), 2.92 (br s, 1H), 2.23-2.10 (m, 1H), 1.82 (br t, J=11.2 Hz, 1H), 1.72-1.63 (m, 1H), 1.58-1.51 (m, 1H), 1.49-1.42 (m, 9H).

### Step 2: Synthesis of Intermediate 1-4

Solution 1: Intermediate 1-3 (0.77 g, 2.60 mmol, 1 eq) in TFE (192.5 mL). The fixed bed (volume: 5 mL) was packed with granular catalyst 10% Ru/SiO₂ (3 g). The H₂ back pressure regulator was adjusted to 2.5 MPa and the flow rate of H₂ was 20 mL/min. Then the solution 1 was pumped (0.303 mL/min) to the fixed bed (6.350(¼″) mm, 1 mL, 100° C.). The reaction mixture was collected from the reactor output. The reaction mixture was concentrated in a vacuum to give Intermediate 1-4 (0.7 g, 2.36 mmol, 90.91% yield) as a yellow oil. LCMS (Method E): Rt=0.415 min, [M+H⁺=297.4. ¹H NMR (400 MHz, CHLOROFORM-d) δ=4.27-3.97 (m, 2H), 3.73-3.48 (m, 2H), 3.31-3.01 (m, 5H), 2.72-2.55 (m, 1H), 1.99-1.77 (m, 4H), 1.76-1.62 (m, 2H), 1.57-1.32 (m, 12H).

### Step 3: Synthesis of Intermediate 1-6

To a solution of Intermediate 1-4 (0.6 g, 2.02 mmol, 1 eq) in ACN (6 mL) was added Intermediate 1-5 (896.49 mg, 2.02 mmol, 1 eq) and DIEA (784.86 mg, 6.07 mmol, 1.06 mL, 3 eq), and the mixture was stirred at 40° C. for 12 hr. The mixture diluted with water (20 mL), and extracted with EA (20 mL*2). The combined organic layers were washed with brine (20 mL), dried over Na₂SO₄, filtered and concentrated under reduced pressure to give a residue. The crude product was dissolved in MeOH (5 mL) and purified by Prep-HPLC (column: Phenomenex luna C18 150*40 mm*15 um; mobile phase: [water (FA)-ACN]; gradient: 15%-45% B over 22 min) and the eluent was concentrated to remove ACN. The aqueous phase was lyophilized to give Intermediate 1-6 (0.8 g, 987.01 μmol, 48.76% yield, 86.71% purity) as a white solid. LCMS (Method E): Rt=0.462 min, [M+H]⁺=703.5. ¹H NMR (400 MHz, CHLOROFORM-d) δ=10.79-10.36 (m, 1H), 8.60-8.39 (m, 1H), 7.89-7.68 (m, 3H), 7.34 (m, 2H), 7.06 (m, 1H), 4.30 (s, 2H), 4.12-4.02 (m, 1H), 3.94-3.48 (m, 7H), 3.43-2.75 (m, 12H), 2.69-2.57 (m, 1H), 2.57-2.29 (m, 2H), 1.99-1.89 (m, 2H), 1.80-1.52 (m, 3H), 1.46 (m, 9H).

### Step 4: Synthesis of Intermediate 1-7

A solution of Intermediate 1-6 (0.7 g, 863.63 μmol, 1 eq) in HCl/MeOH (2 M, 14 mL) was stirred at 25° C. for 1 h. The reaction mixture was concentrated in a vacuum to give Intermediate 1-7 (0.7 g, crude, HCl salt) as a white solid. The residue was used for the next step. LCMS (method E): Rt=0.374 min, [M+H]⁺=603.4.

### Step 5: Synthesis of Intermediate 1-9

To a solution of Intermediate 1-7 (0.7 g, 1.10 mmol, 1 eq, HCl salt) in DMF (14 mL) was added Intermediate 1-8 (281.82 mg, 1.10 mmol, 1 eq), EDCI (629.85 mg, 3.29 mmol, 3 eq), HOAt (149.07 mg, 1.10 mmol, 153.20 μL, 1 eq) and NMM (886.20 mg, 8.76 mmol, 963.26 μL, 8 eq) and the mixture was stirred at 25° C. for 1 hr. The mixture diluted with water (20 mL), and extracted with EA (20 mL*2). The combined organic layers were washed with brine (20 mL), dried over Na₂SO₄, filtered and concentrated under reduced pressure to give crude product. The crude product was purified by reversed-phase HPLC (0.1% FA condition) and concentrated to remove organic solvents. The residual aqueous solution was extracted with EA (20 mL*2). The combined organic layers were washed with brine (20 mL), dried over Na₂SO₄, filtered and concentrated under reduced pressure to give Intermediate 1-9 (0.7 g, 830.52 μmol, 75.83% yield, 99.9% purity) as a white solid. LCMS (Method E): Rt=0.502 min, [M+H]⁺=842.5. SFC: Rt=4.506 min/5.660 min.

### Step 6: Synthesis of Intermediate 1-10

A solution of Intermediate 1-9 (0.6 g, 712.58 μmol, 1 eq) in HCl/MeOH (2 M, 12 mL) was stirred at 25° C. for 1 h. The reaction mixture was concentrated in a vacuum to give Intermediate 1-10 (0.6 g, crude, HCl salt) as a white solid. LCMS (Method E): Rt=0.405 min, [M+H]⁺=742.4.

### Step 7: Synthesis of I-987

To a solution of Intermediate 1-10 (0.4 g, 539.16 μmol, 1 eq, HCl salt) in DMF (8 mL) was added Intermediate 1-11 (165.72 mg, 539.16 μmol, 1 eq), EDCI (310.07 mg, 1.62 mmol, 3 eq), HOAt (73.39 mg, 539.16 μmol, 75.42 μL, 1 eq) and NMM (436.28 mg, 4.31 mmol, 474.21 μL, 8 eq), and the mixture was stirred at 25° C. for 1 hr. The mixture diluted with water (20 mL), and extracted with EA (20 mL*2). The combined organic layers were washed with brine (20 mL), dried over Na₂SO₄, filtered and concentrated under reduced pressure to give crude product. The crude product was dissolved in MeOH (3 mL) and purified by Prep-HPLC (column: Phenomenex luna C18 150*40 mm*15 um; mobile phase: [water (FA)-ACN]; gradient: 13%-43% B over 22 min) and the eluent was concentrated to remove ACN. The aqueous phase was lyophilized to give I-987 (260 mg, 238.53 μmol, 53.96% yield, 98.83% purity, FA salt) as a white solid. LCMS (Method E): Rt=0.53 min, [M+H]⁺=1031.5. SFC: Rt=2.13 min/2.58 min ¹H NMR (400 MHz, METHANOL-d4) δ=8.41 (s, 1H), 8.37 (m, 1H), 7.94 (m, 1H), 7.92-7.79 (m, 2H), 7.60-7.43 (m, 3H), 7.37 (m, 1H), 7.30-7.08 (m, 2H), 4.85-4.42 (m, 5H), 4.39 (s, 2H), 4.32-4.16 (m, 1H), 4.09-3.42 (m, 10H), 3.29-2.63 (m, 10H), 2.21-1.56 (m, 18H), 1.49-0.99 (m, 16H). ¹⁹F NMR (377 MHz, METHANOL-d4) δ=−120.19-−121.06 (m, 1F), −121.06-−122.48 (m, 1F).

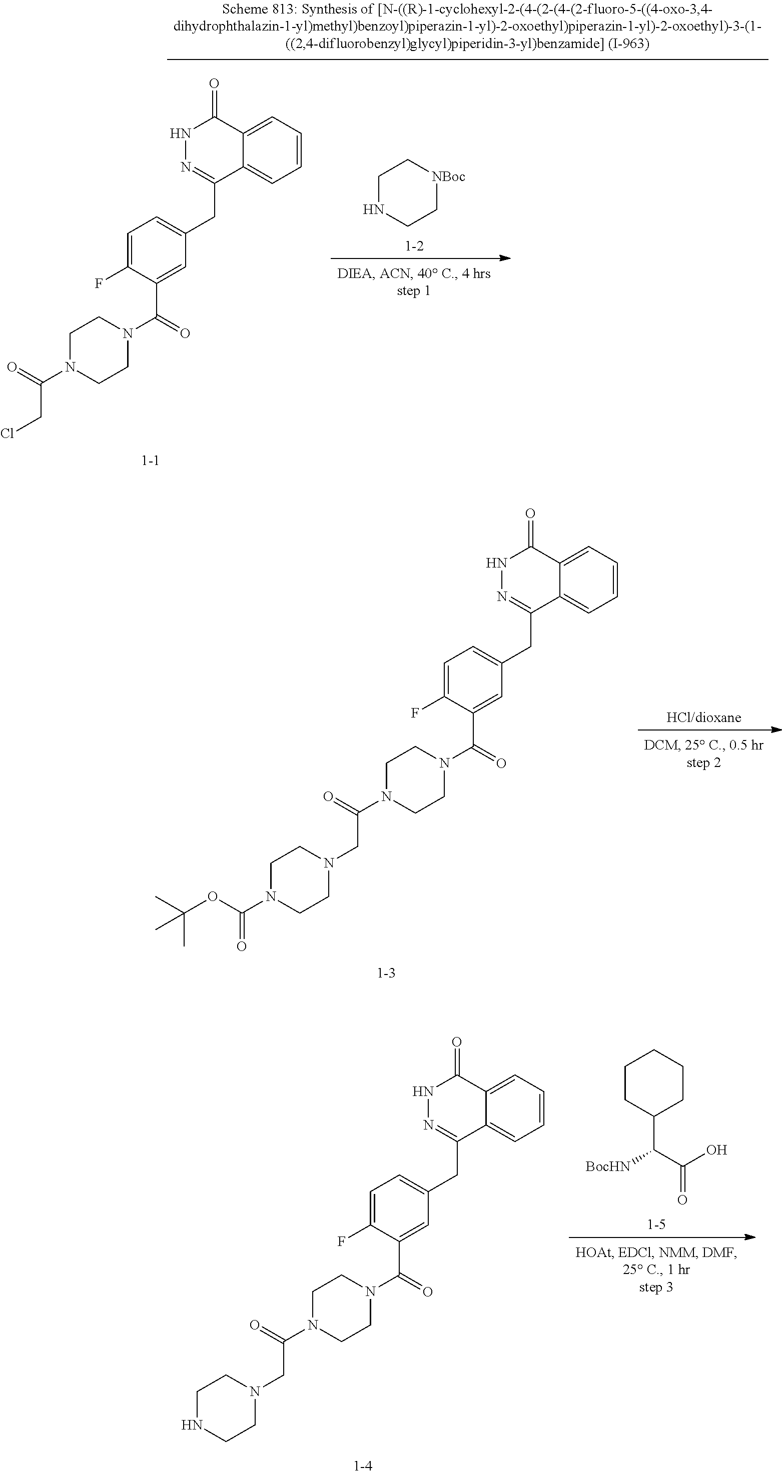

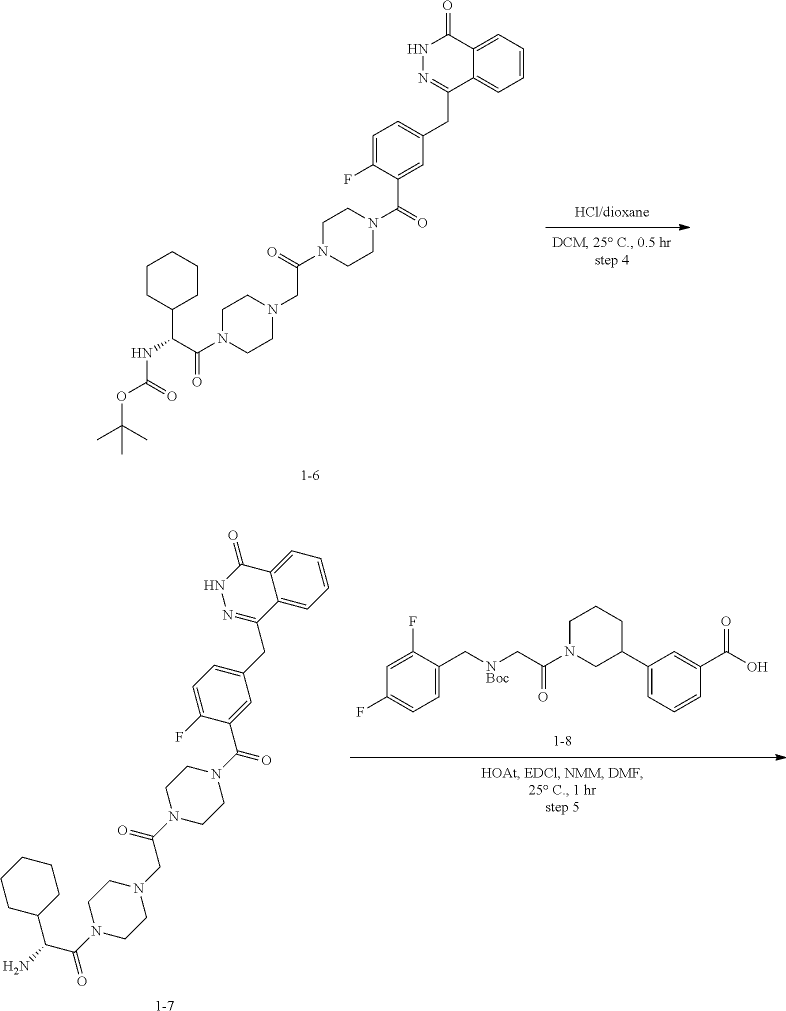 [SMILES: CC(C)(C)OC(=O)N[C@@H](C(=O)N1CCN(CC(=O)N2CCN(C(=O)c3cc(Cc4n[nH]c(=O)c5ccccc45)ccc3F)CC2)CC1)C1CCCCC1 ; "1-6" ; N[C@@H](C(=O)N1CCN(CC(=O)N2CCN(C(=O)c3cc(Cc4n[nH]c(=O)c5ccccc45)ccc3F)CC2)CC1)C1CCCCC1 ; CC(C)(C)OC(=O)N(CC(=O)N1CCCC(c2cccc(C(=O)O)c2)C1)Cc1ccc(F)cc1F ; "1-7"]

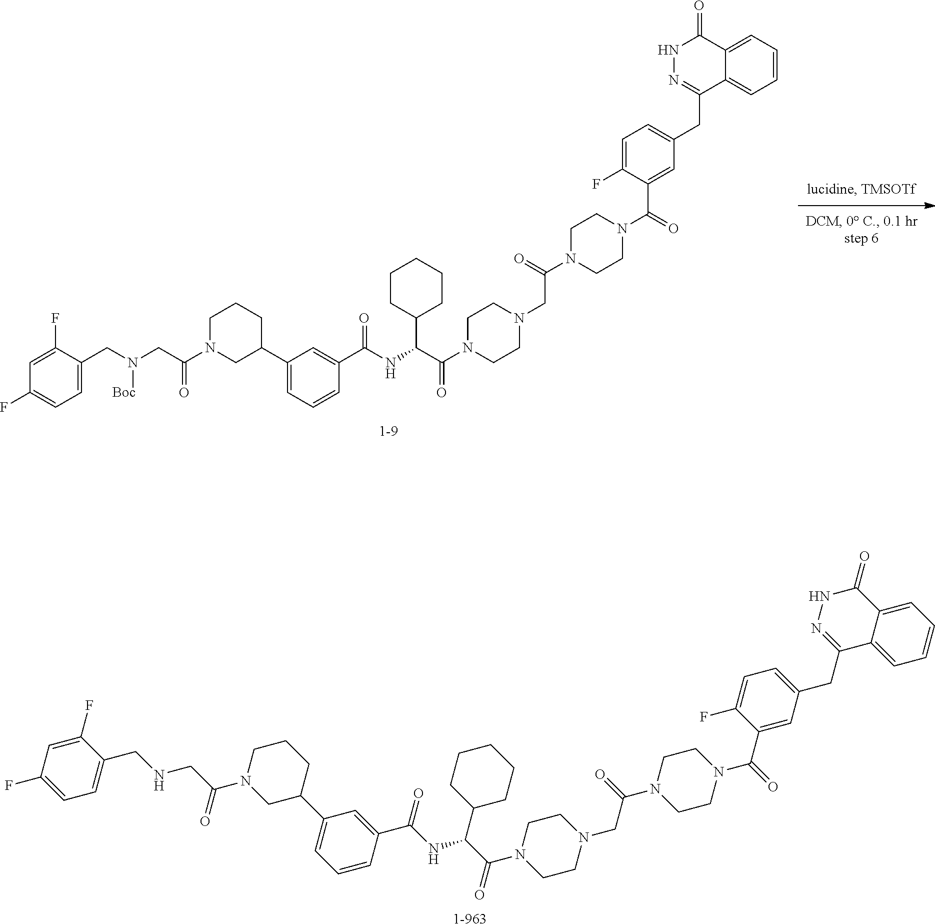

### Step 1: Synthesis of Intermediate 1-3.

To a solution of Intermediate 1-1 (2 g, 4.52 mmol, 1 eq) and Intermediate 1-2 (841.11 mg, 4.52 mmol, 1 eq) in ACN (20 mL) was added DIEA (1.75 g, 13.55 mmol, 2.36 mL, 3 eq). The mixture was stirred at 40° C. for 4 hr. The reaction mixture was diluted with H₂O (10 mL) and extracted with EA (10 mL*3). The combined organic layers were dried over Na₂SO₄, filtered and concentrated under reduced pressure to give a residue. The residue was purified by flash silica gel chromatography (ISCO; 40 g SepaFlash Silica Flash Column, Eluent of 0˜15% Methanol/Ethyl acetate gradient @60 mL/min), and the eluent was concentrated under reduced pressure to give a residue. Intermediate 1-3 (2.6 g, 3.80 mmol, 84.12% yield, 86.593% purity) was obtained as a yellow solid. LCMS (Method D): Retention time: 0.302 min, (M+H)=593.2. ¹H NMR (400 MHz, CHLOROFORM-d) δ=10.70 (d, J=16.8 Hz, 1H), 8.51-8.44 (m, 1H), 7.84-7.66 (m, 3H), 7.41-7.27 (m, 2H), 7.12-6.97 (m, 1H), 4.29 (s, 2H), 3.91-3.65 (m, 4H), 3.63-3.53 (m, 2H), 3.47-3.37 (m, 4H), 3.36-3.13 (m, 4H), 2.45 (d, J=16.1 Hz, 4H), 1.46 (d, J=1.2 Hz, 9H). ¹⁹F NMR (376 MHz, CHLOROFORM-d) δ=−117.61.

### Step 2: Synthesis of Intermediate 1-4.

To a solution of Intermediate 1-3 (2.5 g, 4.22 mmol, 1 eq) in DCM (15 mL) was added HCl/dioxane (2 M, 15 mL, 7.11 eq). The mixture was stirred at 25° C. for 0.5 hr. The reaction mixture was concentrated under reduced pressure to give a residue. The crude product was used in the next step without further purification. Intermediate 1-4 (2.2 g, crude, HCl salt) was obtained as a white solid. LCMS (Method D): Retention time: 0.265 min, (M+H)=493.6.

### Step 3: Synthesis of Intermediate 1-6.

To a solution of Intermediate 1-5 (1.07 g, 4.16 mmol, 1 eq) and Intermediate 1-4 (2.2 g, 4.16 mmol, 1 eq, HCl salt) in DMF (20 mL) was added HOAt (566.05 mg, 4.16 mmol, 581.76 μL, 1 eq) and EDCI (2.39 g, 12.48 mmol, 3 eq) and NMM (2.10 g, 20.79 mmol, 2.29 mL, 5 eq). The mixture was stirred at 25° C. for 1 hr. The reaction mixture was diluted with H₂O (80 mL) and the insoluble material was filtered to give a residue. The residue was purified by flash silica gel chromatography (ISCO; 40 g SepaFlash Silica Flash Column, Eluent of 0-15% Methanol/Ethyl acetate gradient @60 mL/min), and the eluent was concentrated under reduced pressure to give a residue. Intermediate 1-6 (2.2 g, 2.92 mmol, 70.25% yield, 97.182% purity) was obtained as a yellow solid. LCMS (Method D): Retention time: 0.372 min, (M+H)=732.4. SFC: Retention time: 0.533 min. ¹H NMR (400 MHz, CHLOROFORM-d) δ=10.72-10.56 (m, 1H), 8.51-8.43 (m, 1H), 7.82-7.62 (m, 3H), 7.33 (d, J=4.8 Hz, 2H), 7.05 (s, 1H), 5.33 (d, J=8.4 Hz, 1H), 4.49-4.38 (m, 1H), 4.29 (s, 2H), 3.84-3.66 (m, 4H), 3.65-3.54 (m, 5H), 3.41-3.14 (m, 4H), 2.53 (d, J=13.6 Hz, 4H), 1.82-1.67 (m, 5H), 1.42 (s, 9H), 1.29-0.92 (m, 6H). ¹⁹F NMR (376 MHz, CHLOROFORM-d) δ=−117.63.

### Step 4: Synthesis of Intermediate 1-7.

To a solution of Intermediate 1-6 (500 mg, 683.20 μmol, 1 eq) in DCM (3 mL) was added HCl/dioxane (2 M, 3 mL). The mixture was stirred at 25° C. for 0.5 hr. The reaction mixture was concentrated under reduced pressure to give a residue. The crude product was used in the next step without further purification. Intermediate 1-7 (450 mg, 601.88 μmol, 88.10% yield, 89.372% purity, HCl salt) was obtained as a pale yellow solid. LCMS (Method D): Retention time: 0.258 min, (M+H)=632.3.

### Step 5: Synthesis of Intermediate 1-9.

To a solution of Intermediate 1-8 (146.22 mg, 299.31 μmol, 1 eq) and Intermediate 1-7 (200 mg, 299.31 μmol, 1 eq, HCl salt) in DMF (2 mL) was added HOAt (40.74 mg, 299.31 μmol, 41.87 μL, 1 eq) EDCI (172.14 mg, 897.94 μmol, 3 eq) and NMM (151.37 mg, 1.50 mmol, 164.54 μL, 5 eq). The mixture was stirred at 25° C. for 1 hr. The reaction mixture was diluted with H₂O (2 mL) and extracted with EA (5 mL*3). The combined organic layers were washed with brine (5 mL*3), dried over Na₂SO₄, filtered and concentrated under reduced pressure to give a residue. The residue was purified by flash silica gel chromatography (ISCO; 20 g SepaFlash Silica Flash Column, Eluent of 0˜15% Methanol/Ethyl acetate gradient @60 mL/min), the eluent was concentrated under reduced pressure to give a residue. Intermediate 1-9 (210 mg, 190.52 μmol, 63.65% yield, 100% purity) was obtained as a yellow solid. LCMS (Method D): Retention time: 0.458 min, (M+H)=1102.7. ¹H NMR (400 MHz, METHANOL-d4) δ=8.40-8.33 (m, 1H), 7.95 (d, J=8.0 Hz, 1H), 7.91-7.80 (m, 2H), 7.78-7.67 (m, 2H), 7.50-7.33 (m, 5H), 7.20-7.13 (m, 1H), 6.99-6.90 (m, 2H), 4.61-4.46 (m, 8H), 4.38 (s, 2H), 3.95-3.65 (m, 10H), 3.61-3.48 (m, 4H), 2.81-2.67 (m, 2H), 2.61-2.45 (m, 4H), 2.08-2.01 (m, 1H), 1.94-1.83 (m, 4H), 1.81-1.74 (m, 2H), 1.72-1.67 (m, 2H), 1.44 (s, 9H), 1.34-1.25 (m, 4H), 1.15-1.07 (m, 2H).

### Step 6: Synthesis of I-963.

To a solution of Intermediate 1-9 (150 mg, 136.09 μmol, 1 eq) in DCM (1 mL) was added 2,6-dimethylpyridine (4.37 mg, 40.83 μmol, 4.75 μL, 0.3 eq) and trimethylsilyl trifluoromethanesulfonate (45.37 mg, 204.13 μmol, 36.89 μL, 1.5 eq) at 0° C. The mixture was stirred at 0° C. for 0.1 hr. The reaction mixture was concentrated under reduced pressure to give a residue. The residue was purified by prep-HPLC (column: CD02-Waters Xbidge BEH C18 150*25*10 um; mobile phase: [water(NH₃H₂O)-ACN]; gradient: 36%-66% B over 12 min) and the eluent was concentrated and lyophilized to give the desired product. I-963 (25.94 mg, 25.65 μmol, 18.85% yield, 99.083% purity) was obtained as a white solid. LCMS (Method D): Retention time: 0.385 min, (M+H)=1002.5. SFC: Retention time: 2.073, 2.460 min. ¹H NMR (400 MHz, METHANOL-d4) δ=8.39-8.33 (m, 1H), 7.97-7.92 (m, 1H), 7.90-7.79 (m, 2H), 7.77-7.68 (m, 2H), 7.51-7.40 (m, 4H), 7.37 (s, 1H), 7.20-7.11 (m, 1H), 6.99-6.88 (m, 2H), 4.91 (s, 1H), 4.56 (d, J=16.8 Hz, 1H), 4.38 (s, 2H), 3.87 (s, 1H), 3.81 (d, J=10.0 Hz, 4H), 3.74 (d, J=7.2 Hz, 3H), 3.69 (d, J=11.2 Hz, 2H), 3.62-3.49 (m, 5H), 3.48-3.40 (m, 1H), 3.35 (d, J=2.0 Hz, 1H), 3.25 (s, 1H), 3.17-3.05 (m, 1H), 2.86-2.69 (m, 2H), 2.62-2.43 (m, 4H), 2.03 (d, J=6.8 Hz, 1H), 1.98-1.73 (m, 7H), 1.69 (d, J=11.2 Hz, 2H), 1.64-1.51 (m, 1H), 1.34-1.20 (m, 3H), 1.15-1.04 (m, 2H). ¹⁹F NMR (376 MHz, METHANOL-d4) δ=−113.35, −116.24, −120.75.

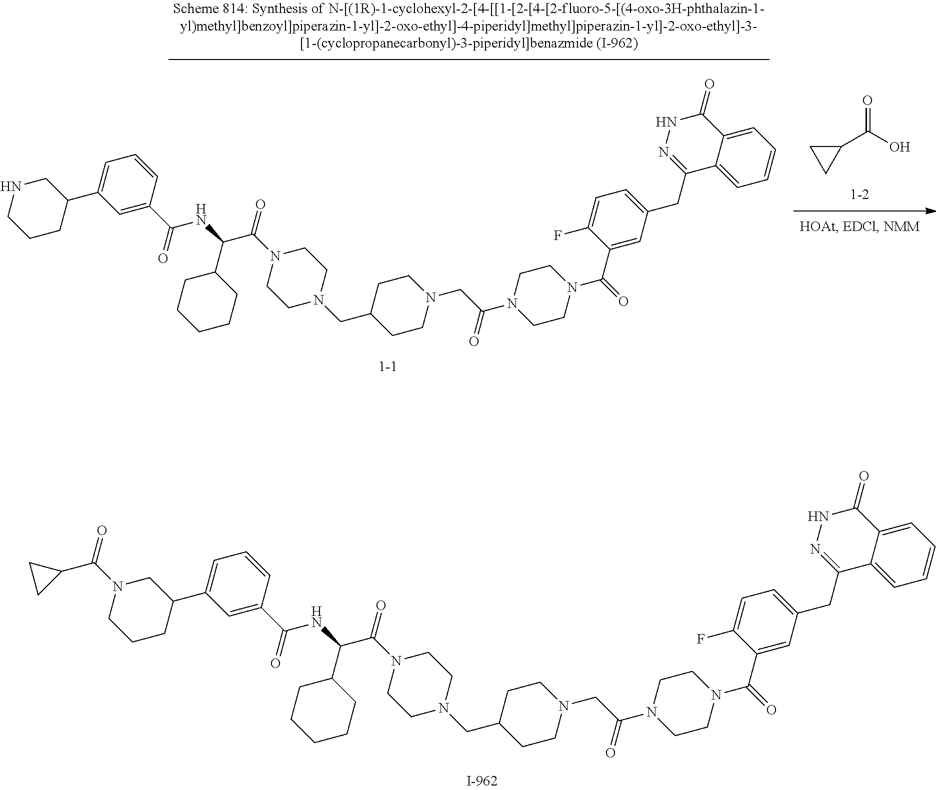

To a mixture of Intermediate 1-1 (50 mg, 54.58 μmol, 1 eq) and Intermediate 1-2 (7.05 mg, 81.87 μmol, 6.48 μL, 1.5 eq) in DMF (1 mL) was added HOAt (7.43 mg, 54.58 μmol, 7.63 μL, 1 eq), EDCI (31.39 mg, 163.73 μmol, 3 eq), and NMM (27.60 mg, 272.89 μmol, 30.00 μL, 5 eq), and then the mixture was stirred at 25° C. for 1 hour. The reaction mixture was quenched with water (20 mL) and extracted with EA (5 mL*2). The combined organic layers were concentrated under reduced pressure to give a residue. The crude product was purified by prep-HPLC (column: Phenomenex luna C18 150*25 mm*10 um; mobile phase: [water (FA)-ACN]; gradient: 15%-45% B over 8 min). The eluent was concentrated under reduced pressure to remove MeCN and then lyophilized to give I-962 (11.30 mg, 11.37 μmol, 20.83% yield, 99% purity) as a white solid. LCMS (Method D): Retention time: 0.352 min, (M+H)=984.4. SFC: Rt=2.881, Rt=3.544. ¹H NMR (400 MHz, METHANOL-d4) δ=8.67-8.44 (m, 1H), 8.39 (d, J=7.6 Hz, 1H), 7.96 (d, J=2.8 Hz, 1H), 7.94-7.83 (m, 2H), 7.82-7.68 (m, 2H), 7.57-7.35 (m, 4H), 7.19 (t, J=8.8 Hz, 1H), 4.62-4.56 (m, 1H), 4.46-4.36 (m, 3H), 3.93-3.68 (m, 8H), 3.66-3.54 (m, 3H), 3.46 (d, J=1.6 Hz, 1H), 3.27 (d, J=4.0 Hz, 6H), 2.90-2.62 (m, 4H), 2.59-2.40 (m, 4H), 2.29 (s, 2H), 2.09 (d, J=12.4 Hz, 1H), 2.01-1.69 (m, 12H), 1.54-1.20 (m, 6H), 1.18-1.06 (m, 2H), 0.96-0.77 (m, 4H). ¹⁹F NMR (376 MHz, METHANOL-d4) δ=−120.77.

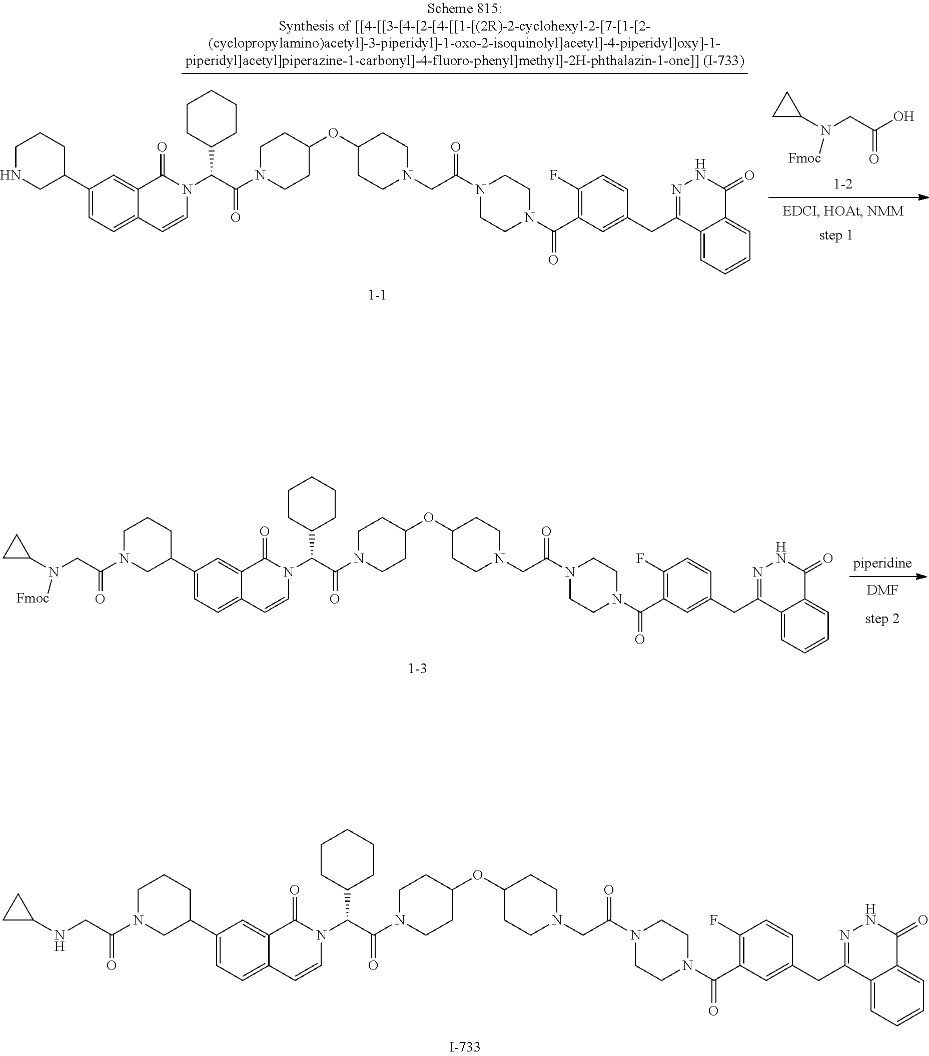

### Step 1: Synthesis of Intermediate 1-3

To a solution of Intermediate 1-1 (85 mg, 90.32 μmol, 1 eq) in DMF (3 mL) was added Intermediate 1-2 (30.47 mg, 90.32 μmol, 1 eq), EDCI (34.63 mg, 180.63 μmol, 2 eq), HOAt (12.29 mg, 90.32 μmol, 12.63 μL, 1 eq) and NMM (45.68 mg, 451.58 μmol, 49.65 μL, 5 eq). The mixture was stirred at 25° C. for 1 hr. The residue was used in the next step without work up. A solution of Intermediate 1-3 (110 mg, crude) in DMF (3 mL) as a brown liquid was obtained. LCMS (Method G): Rt=0.824 min, [M+H]⁺=1261.9.

### Step 2: Synthesis of I-733

To a solution of Intermediate 1-3 (110 mg, 87.27 μmol, 1 eq) in DMF (3 mL) was added piperidine (646.65 mg, 7.59 mmol, 0.75 mL, 87.03 eq). The mixture was stirred at 25° C. for 0.5 hr. The mixture was diluted with water (5 ml) and extracted with EtOAc (10 ml*3). The organic layer was concentrated under vacuum. The residue was purified by prep-HPLC (column: Waters Xbridge 150*25 mm*5 um; mobile phase: [water (ammonia hydroxide v/v)-ACN]; gradient: 42%-72% B over 11 min) and dried by lyophilization. I-733 (25 mg, 23.90 μmol, 27.39% yield, 99.27% purity) was obtained as a white solid. LCMS (Method G): Rt=0.680 min, [M+H]⁺=1038.6. SCF: Rt=3.398 min, 3.872 min. ¹H NMR (400 MHz, DMSO-d6) δ=12.64-12.55 (m, 1H), 8.28-8.22 (m, 1H), 8.17-8.09 (m, 1H), 7.98-7.93 (m, 1H), 7.96-7.83 (m, 1H), 7.85-7.79 (m, 1H), 7.73-7.61 (m, 2H), 7.45-7.34 (m, 3H), 7.26-7.19 (m, 1H), 6.72-6.65 (m, 1H), 5.76-5.67 (m, 1H), 4.51-4.40 (m, 1H), 4.32 (br d, J=4.0 Hz, 2H), 4.01-3.80 (m, 3H), 3.66-3.53 (m, 4H), 3.51-3.42 (m, 3H), 3.35 (br d, J=3.6 Hz, 2H), 3.17-3.02 (m, 6H), 2.64-2.60 (m, 1H), 2.45-2.36 (m, 2H), 2.20-2.09 (m, 3H), 2.04-1.90 (m, 2H), 1.83-1.52 (m, 12H), 1.49-1.20 (m, 6H), 1.17-1.02 (m, 5H), 0.97-0.81 (m, 2H), 0.37-0.28 (m, 2H), 0.26-0.17 (m, 2H). ¹⁹F NMR (377 MHz, DMSO-d₆) δ=−119.14-−120.96 (m, 1F).

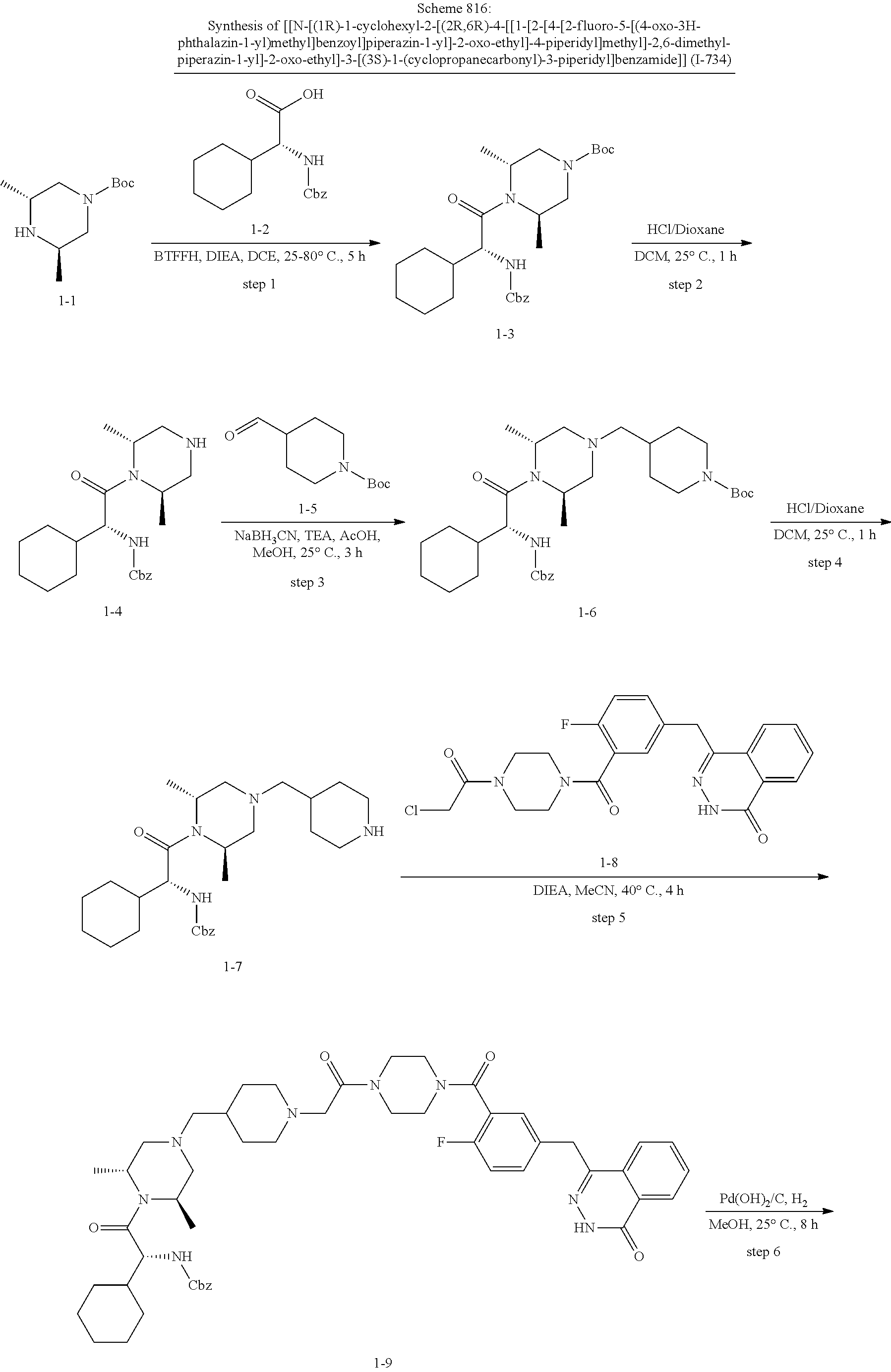

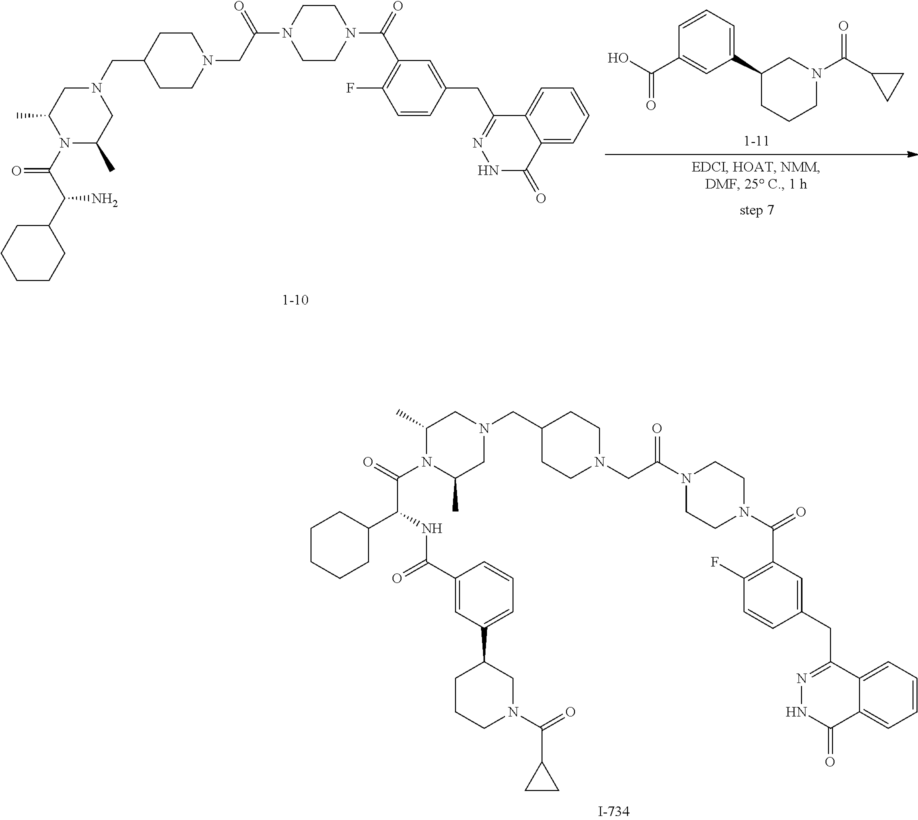 [SMILES: C[C@@H]1CN(CC2CCN(CC(=O)N3CCN(C(=O)c4cc(Cc5n[nH]c(=O)c6ccccc56)ccc4F)CC3)CC2)C[C@@H](C)N1C(=O)[C@H](N)C1CCCCC1 ; O=C(O)c1cccc([C@@H]2CCCN(C(=O)C3CC3)C2)c1 ; C[C@@H]1CN(CC2CCN(CC(=O)N3CCN(C(=O)c4cc(Cc5n[nH]c(=O)c6ccccc56)ccc4F)CC3)CC2)C[C@@H](C)N1C(=O)[C@H](NC(=O)c1cccc([C@@H]2CCCN(C(=O)C3CC3)C2)c1)C1CCCCC1]

### Step 1: Synthesis of Intermediate 1-3

To a mixture of Intermediate 1-2 (5.44 g, 18.67 mmol, 2 eq) and BTFFH (11.80 g, 37.33 mmol, 4 eq) in DCE (20 mL) under N₂ was added DIEA (6.03 g, 46.66 mmol, 8.13 mL, 5 eq). The mixture was stirred under N₂ at 25° C. for 30 min. Intermediate 1-1 (2 g, 9.33 mmol, 1 eq) was added to the mixture and then heated to 80° C. and stirred for 3 h. The reaction mixture was diluted with water (50 mL) and extracted with DCM (30 mL*3). The combined organic layers were dried over Na₂SO₄, filtered and concentrated under reduced pressure to give a residue. The residue was purified by reversed-phase HPLC (0.1% FA condition). The eluent was concentrated under reduced pressure to give Intermediate 1-3 (4.4 g, 9.02 mmol, 96.69% yield) as a yellow solid. LCMS (Method D): Retention time: 0.507 min, [M+Na]⁺=510.2. SCF: Retention time: 0.804 min.

### Step 2: Synthesis of Intermediate 1-4

A mixture of Intermediate 1-3 (4.3 g, 8.82 mmol, 1 eq) in HCl/dioxane (2 M, 43.00 mL) was stirred at 25° C. for 2.5 hr. The reaction mixture was concentrated under reduced pressure to afford a residue. The residue was used in the next step without further purification. Intermediate 1-4 (3.75 g, crude, HCl salt) was obtained as a yellow oil. LCMS (Method D): Retention time: 0.282 min, [M+H]⁺=388.6.

### Step 3: Synthesis of Intermediate 1-6

To a solution of Intermediate 1-4 (3.74 g, 8.82 mmol, 1 eq, HCl salt) in MeOH (40 mL) was added TEA (5.36 g, 52.93 mmol, 7.37 mL, 6 eq) and it was stirred at 25° C. for 10 min. Then AcOH (3.18 g, 52.93 mmol, 3.03 mL, 6 eq) and Intermediate 1-5 (4.70 g, 22.05 mmol, 2.5 eq) was added. The mixture was stirred at 25° C. for 30 min, then NaBH₃CN (2.22 g, 35.29 mmol, 4 eq) was added. The mixture was stirred at 25° C. for 2.5 h. The reaction mixture was diluted with water (100 mL) and extracted with DCM (40 mL*3). The combined organic layers were dried over Na₂SO₄, filtered and concentrated under reduced pressure to give a residue. The residue was purified by reversed-phase HPLC (0.1% FA condition). The eluent was concentrated under reduced pressure to give Intermediate 1-6 (3.72 g, 6.04 mmol, 68.51% yield, 95% purity) as a yellow solid. LCMS (Method D): Retention time: 0.378 min, [M+H]⁺=585.4.

### Step 4: Synthesis of Intermediate 1-7

A mixture of Intermediate 1-6 (3.7 g, 6.33 mmol, 1 eq) in HCl/dioxane (2 M, 37 mL, 1.00 eq) was stirred at 25° C. for 2.5 hr. The reaction mixture was concentrated under reduced pressure to give a residue. The residue was used for the next step without further purification. Intermediate 1-7 (3.7 g, crude, HCl salt) was obtained as a yellow oil. LCMS: Retention time: 0.299 min, [M+H]⁺=485.3.

### Step 5: Synthesis of Intermediate 1-9

To a solution of Intermediate 1-7 (3.7 g, 7.10 mmol, 2 eq, HCl salt) and Intermediate 1-8 (1.57 g, 3.55 mmol, 1 eq) in ACN (37 mL) was added DIEA (917.61 mg, 7.10 mmol, 1.24 mL, 2 eq). The mixture was stirred at 40° C. for 4 hr. The reaction mixture was diluted with water (50 mL) and extracted with DCM (40 mL*3). The combined organic layers were dried over Na₂SO₄, filtered and concentrated under reduced pressure to give a residue. The residue was purified by flash silica gel chromatography (ISCO; 120 g SepaFlash Silica Flash Column, Eluent of 0-50% DCM/MeOH@40 mL/min). The eluent was concentrated under reduced pressure to afford Intermediate 1-9 (1.8 g, 1.89 mmol, 53.25% yield, 93.575% purity) as a yellow oil. LCMS (Method D): Retention time: 0.324 min, [M+H]⁺=891.6. SCF: Retention time: 0.959 min. ¹H NMR (400 MHz, DMSO-d6) δ=12.60 (s, 1H), 8.26 (d, J=7.6 Hz, 1H), 8.03-7.94 (m, 1H), 7.91-7.78 (m, 2H), 7.53 (d, J=9.2 Hz, 1H), 7.44 (s, 1H), 7.41-7.27 (m, 6H), 7.24 (t, J=9.2 Hz, 1H), 5.11-4.93 (m, 2H), 4.33 (s, 2H), 4.12-4.08 (m, 4H), 3.68-3.44 (m, 5H), 3.36 (d, J=4.4 Hz, 1H), 3.17 (s, 4H), 2.78 (s, 2H), 2.40-2.09 (m, 4H), 1.95 (s, 1H), 1.80-1.49 (m, 9H), 1.41 (s, 1H), 1.34-1.23 (m, 6H), 1.20-0.97 (m, 6H), 0.95-0.82 (m, 2H).

### Step 6: Synthesis of Intermediate 1-10

To a solution of Intermediate 1-9 (1.8 g, 2.02 mmol, 1 eq) in MeOH (18 mL) was added Pd(OH)₂/C (900 mg, 1.28 mmol, 20% purity, 6.35e−1 eq) under N₂ atmosphere. The suspension was degassed and purged with H₂ 3 times and stirred under H₂ (15 psi) at 25° C. for 10 hr. The reaction mixture was filtered through a pad of celite and washed with MeOH (20 mL*3), then concentrated under reduced pressure to give a residue. The residue was purified by reversed-phase HPLC (0.1% FA condition). The eluent was lyophilized to give Intermediate 1-10 (700 mg, 884.47 μmol, 43.79% yield, 95.6% purity) as a white solid. LCMS (Method D): Retention time: 0.256 min, [M+H]⁺=757.4.

### Step 7: Synthesis of I-734

To a solution of Intermediate 1-11 (144.44 mg, 528.44 μmol, 1 eq) in DMF (4 mL) was added HOAt (71.93 mg, 528.44 μmol, 73.92 μL, 1 eq), EDCI (303.91 mg, 1.59 mmol, 3 eq), NMM (267.25 mg, 2.64 mmol, 290.49 μL, 5 eq) and Intermediate 1-10 (400 mg, 528.44 μmol, 1 eq). The mixture was stirred at 25° C. for 1 hr. The reaction mixture was diluted with water (10 mL) and extracted with DCM (5 mL*3). The combined organic layers were dried over Na₂SO₄, filtered and concentrated under reduced pressure to give a residue. The residue was purified by flash silica gel chromatography (ISCO; 40 g SepaFlash Silica Flash Column, Eluent of 0˜100% DCM/MeOH @40 mL/min). The eluent was concentrated under reduced pressure to give a residue. The residue was purified by reversed-phase chromatography (Neutral condition). The eluent was lyophilized to give I-734 (220 mg, 216.71 μmol, 41.01% yield, 99.7% purity) as a white solid. LCMS (Method D): Retention time: 0.371 min, [M+H]⁺=1012.5. SCF: Retention time: 1.638 min. ¹H NMR (400 MHz, METHANOL-d4) δ=8.40-8.34 (m, 1H), 7.98-7.91 (m, 1H), 7.91-7.80 (m, 2H), 7.80-7.65 (m, 2H), 7.54-7.34 (m, 4H), 7.16 (t, J=8.8 Hz, 1H), 4.77 (d, J=8.8 Hz, 1H), 4.57 (d, J=9.6 Hz, 1H), 4.42-4.36 (m, 3H), 3.83-3.44 (m, 7H), 3.29-3.11 (m, 4H), 2.92-2.66 (m, 6H), 2.62-2.41 (m, 2H), 2.34-2.24 (m, 2H), 2.13-1.91 (m, 6H), 1.91-1.60 (m, 10H), 1.53-1.40 (m, 7H), 1.35-1.02 (m, 8H), 0.92-0.75 (m, 4H). ¹⁹F NMR (376 MHz, METHANOL-d4) δ=−120.76 (br d, J=28.3 Hz, 1F).

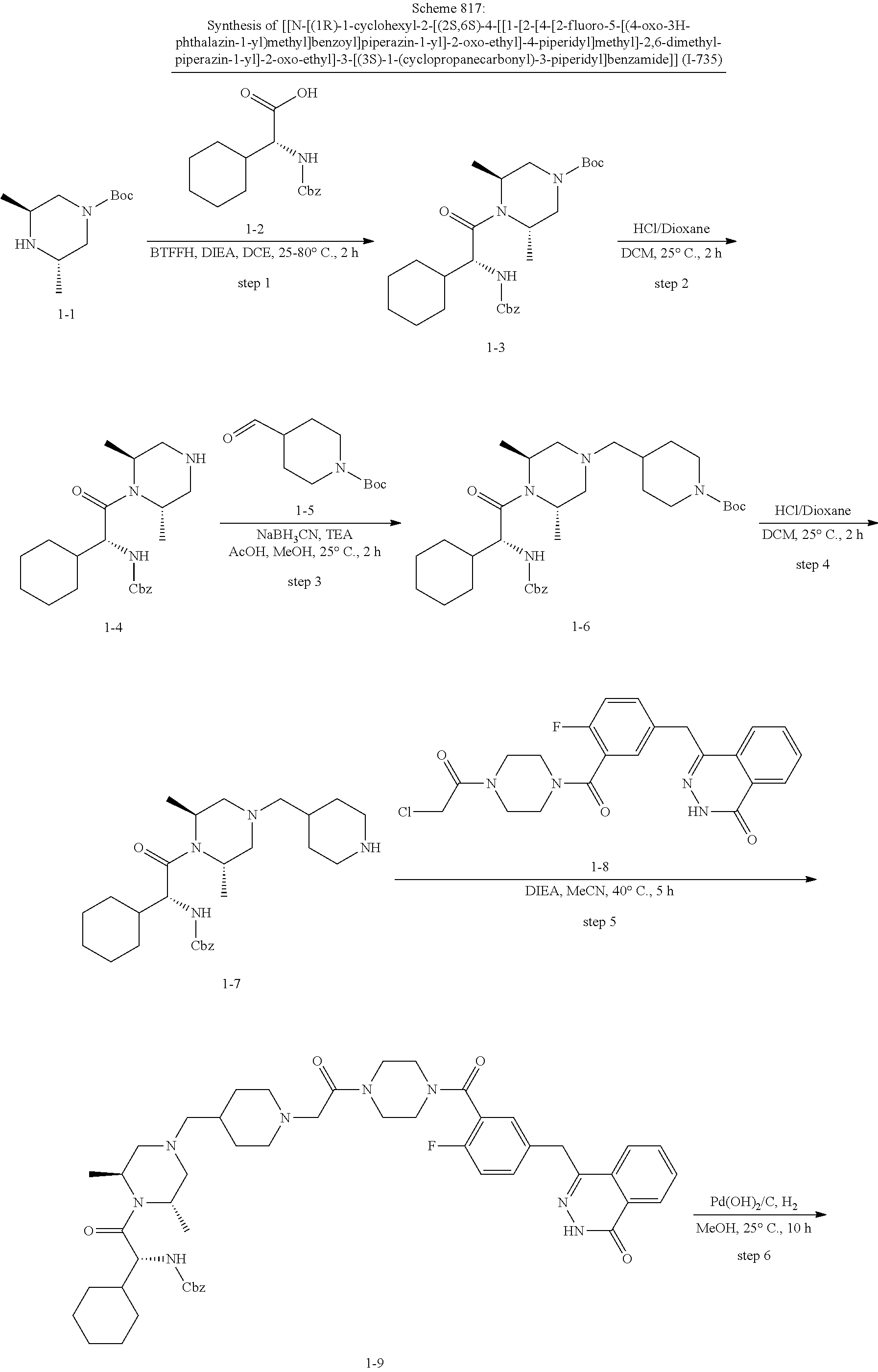

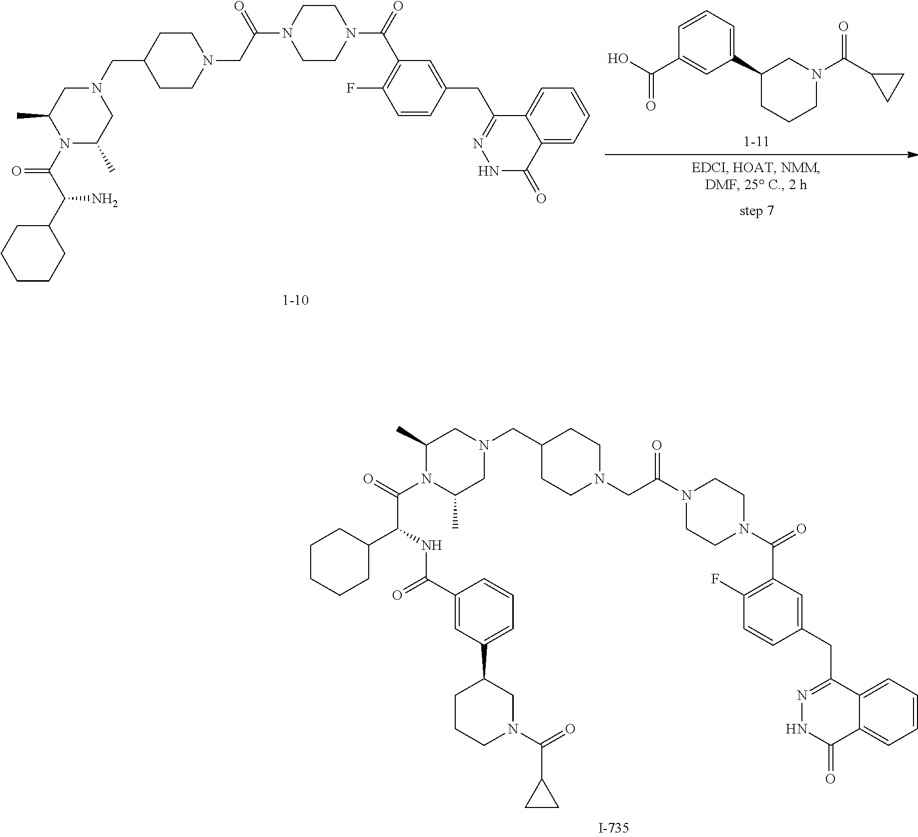 [SMILES: C[C@H]1CN(CC2CCN(CC(=O)N3CCN(C(=O)c4cc(Cc5n[nH]c(=O)c6ccccc56)ccc4F)CC3)CC2)C[C@H](C)N1C(=O)[C@H](N)C1CCCCC1 ; O=C(O)c1cccc([C@@H]2CCCN(C(=O)C3CC3)C2)c1 ; C[C@H]1CN(CC2CCN(CC(=O)N3CCN(C(=O)c4cc(Cc5n[nH]c(=O)c6ccccc56)ccc4F)CC3)CC2)C[C@H](C)N1C(=O)[C@H](NC(=O)c1cccc([C@@H]2CCCN(C(=O)C3CC3)C2)c1)C1CCCCC1]

### Step 1: Synthesis of Intermediate 1-3

To a solution of Intermediate 1-2 (1.36 g, 4.67 mmol, 2 eq) and BTFFH (2.95 g, 9.33 mmol, 4 eq, CAS: 164298-25-3) in DCE (5 mL) under N₂, was added DIEA (1.51 g, 11.67 mmol, 2.03 mL, 5 eq). The mixture was stirred under N₂ at 25° C. for 30 min. Then Intermediate 1-1 (500 mg, 2.33 mmol, 1 eq) was added to the above mixture and it was then stirred at 80° C. for 3.5 h. The reaction mixture was diluted with water (10 mL) and extracted with EA (10 mL*3). The combined organic layers were dried over Na₂SO₄, filtered and concentrated under reduced pressure to give a residue. The crude product was purified by reversed-phase HPLC (0.1% FA condition). The eluent was concentrated under reduced pressure to remove ACN, and then lyophilized to afford Intermediate 1-3 (1.1 g, 2.24 mmol, 96.08% yield, 99.3% purity) as a white solid. LCMS (Method D): Rt=0.516 min, [M+H]⁺=488.1. SCF: Rt: 0.819 min, 0.878 min.

### Step 2: Synthesis of Intermediate 1-4

To a solution of Intermediate 1-3 (1 g, 2.05 mmol, 1 eq) in DCM (2 mL) was added HCl/Dioxane (2 M, 8.00 mL, 7.80 eq). The mixture was stirred at 25° C. for 0.5 hr. The reaction mixture was concentrated under reduced pressure to give Intermediate 1-4 (860 mg, 2.03 mmol, 98.91% yield, HCl salt) as yellow oil which was used in the next step without further purification. LCMS (Method D): Rt=0.332 min, [M+H]⁺=388.2.

### Step 3: Synthesis of Intermediate 1-6

To a solution of Intermediate 1-4 (500 mg, 1.18 mmol, 1 eq, HCl salt) in MeOH (5 mL) was added TEA (716.00 mg, 7.08 mmol, 984.87 μL, 6 eq) and it was stirred at 25° C. for 10 min, then AcOH (424.92 mg, 7.08 mmol, 405.07 μL, 6 eq) and Intermediate 1-5 (628.79 mg, 2.95 mmol, 2.5 eq) was added, then the mixture was stirred at 25° C. for 1 h. Then NaBH₃CN (296.44 mg, 4.72 mmol, 4 eq) was added to the above mixture. The mixture was stirred at 25° C. for 1 h. The reaction mixture was diluted with water (10 mL) and extracted with EA (10 mL*3). The combined organic layers were dried over Na₂SO₄, filtered and concentrated under reduced pressure to give a residue. The residue was purified by reversed-phase HPLC (0.1% FA). The eluent was concentrated under reduced pressure to remove ACN, then lyophilized to afford Intermediate 1-6 (500 mg, 829.36 μmol, 70.33% yield, 97% purity) as a white solid. LCMS (Method D): Rt=0.370 min, [M+H]⁺=585.9. SCF: Rt: 1.211 min, 1.832 min. ¹H NMR (400 MHz, METHANOL-d4) δ=7.46-7.24 (m, 5H), 5.10-4.94 (m, 2H), 4.44-4.24 (m, 1H), 4.17-3.75 (m, 5H), 2.91-2.69 (m, 4H), 1.89-1.56 (m, 10H), 1.41 (s, 17H), 1.24-0.92 (m, 8H).

### Step 4: Synthesis of Intermediate 1-7

To a solution of Intermediate 1-6 (450 mg, 769.51 μmol, 1 eq) in DCM (1 mL) was added HCl/Dioxane (2 M, 3.60 mL, 9.36 eq). The mixture was stirred at 25° C. for 0.5 hr. The residue was concentrated under reduced pressure to give Intermediate 1-7 (400 mg, 767.56 μmol, 99.75% yield, HCl salt) as a yellow oil which was used in the next step without further purification. LCMS (Method D): Rt=0.303 min, [M+H]⁺=485.3.

### Step 5: Synthesis of Intermediate 1-9

To a solution of Intermediate 1-7 (353.02 mg, 677.4 μmol, 2 eq, HCl salt) and Intermediate 1-8 (150 mg, 338.70 μmol, 1 eq) in ACN (1.5 mL) was added DIEA (87.55 mg, 677.40 μmol, 117.99 μL, 2 eq). The mixture was stirred at 40° C. for 1 hr. The residue was diluted with H₂O (5 mL) and extracted with EA (5 mL*3). The combined organic phase was dried over Na₂SO₄, filtered and concentrated under reduced pressure to give a residue. The residue was purified by flash silica gel chromatography (ISCO; 12 g SepaFlash Silica Flash Column, Eluent of 0˜100% Dichloromethane/Methanol@60 mL/min). The eluent was concentrated under reduced pressure to give Intermediate 1-9 (250 mg, 265.69 μmol, 78.44% yield, 94.7% purity) as yellow oil. LCMS (Method D): Rt=0.326 min [M+H]⁺=891.5. SCF: Rt: 0.924 min, 0.981 min.

### Step 6: Synthesis of Intermediate 1-10

To a solution of Intermediate 1-9 (230 mg, 258.11 μmol, 1 eq) in MeOH (10 mL) was added Pd(OH)₂/C (115 mg, 163.78 μmol, 20% purity, 6.35e−1 eq) under N₂ atmosphere. The suspension was degassed and purged with H₂ 3 times. The mixture was stirred under H₂ (15 psi) at 25° C. for 2 hr. The reaction was diluted with MeOH (10 mL*3), filtered and concentrated under reduced pressure to give Intermediate 1-10 (180 mg, 237.80 μmol, 92.13% yield) as a yellow oil which was used in the next step without further purification. LCMS (Method D): Rt=0.262 min, [M+H]⁺=757.5.

### Step 7: Synthesis of I-735

To a solution of Intermediate 1-11 (54.16 mg, 198.16 μmol, 1 eq) in DMF (1.5 mL) was added EDCI (189.94 mg, 990.82 μmol, 5 eq), NMM (200.44 mg, 1.98 mmol, 217.87 μL, 10 eq) and HOAt (53.94 mg, 396.33 μmol, 55.44 μL, 2 eq) followed by Intermediate 1-10 (150 mg, 198.16 μmol, 1 eq). The mixture was stirred at 25° C. for 1 hr. The residue was diluted with H₂O (5 mL) and extracted with EA (5 mL*3). The combined organic phase was dried over Na₂SO₄, filtered and concentrated under reduced pressure to give a residue. The residue was purified by flash silica gel chromatography (ISCO; 12 g SepaFlash Silica Flash Column, Eluent of 0˜100% Dichloromethane/Methanol @60 mL/min). The eluent was concentrated under reduced pressure then lyophilized to give I-735 (83 mg, 81.99 μmol, 41.38% yield) as a white solid. LCMS (Method D): Rt=0.351 min, [M+H]⁺=1012.6. SCF: Rt: 1.607 min, 2.014 min. ¹H NMR (400 MHz, METHANOL-d4) δ=8.37 (d, J=7.2 Hz, 1H), 8.00-7.91 (m, 1H), 7.91-7.80 (m, 2H), 7.80-7.63 (m, 2H), 7.58-7.31 (m, 4H), 7.16 (t, J=9.2 Hz, 1H), 5.06 (br d, J=4.0 Hz, 1H), 4.77 (br d, J=8.4 Hz, 1H), 4.57 (br d, J=10.4 Hz, 1H), 4.39 (s, 3H), 3.87-3.41 (m, 7H), 3.22 (br d, J=9.6 Hz, 4H), 3.00-2.67 (m, 6H), 2.63-2.18 (m, 4H), 2.17-1.89 (m, 6H), 1.88-1.55 (m, 10H), 1.53-1.35 (m, 7H), 1.34-0.98 (m, 8H), 0.96-0.69 (m, 4H). ¹⁹F NMR (377 MHz, METHANOL-d4) δ=−120.76.

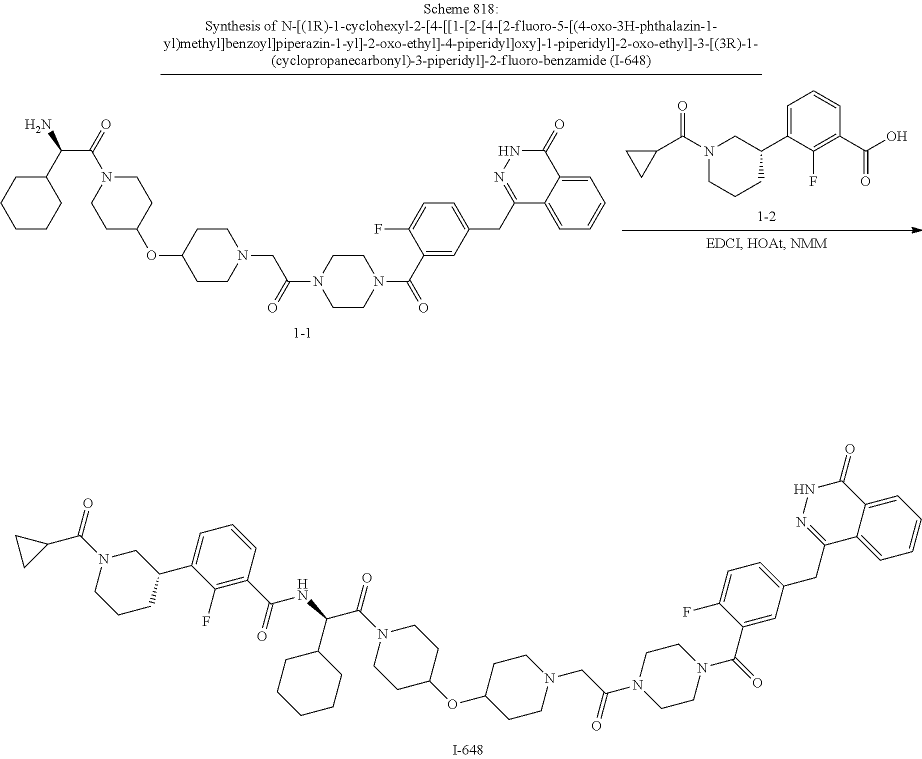 [SMILES: N[C@@H](C(=O)N1CCC(OC2CCN(CC(=O)N3CCN(C(=O)c4cc(Cc5n[nH]c(=O)c6ccccc56)ccc4F)CC3)CC2)CC1)C1CCCCC1 ; O=C(O)c1cccc([C@@H]2CCCN(C(=O)C3CC3)C2)c1F ; O=C(N[C@@H](C(=O)N1CCC(OC2CCN(CC(=O)N3CCN(C(=O)c4cc(Cc5n[nH]c(=O)c6ccccc56)ccc4F)CC3)CC2)CC1)C1CCCCC1)c1cccc([C@@H]2CCCN(C(=O)C3CC3)C2)c1F]

To a solution of intermediate 1-1 (50 mg, 65.25 μmol, 1 eq, HCl salt) and intermediate 1-2 (19.01 mg, 65.25 μmol, 1 eq) in DMF (1 mL) was added EDCI (25.02 mg, 130.49 μmol, 2 eq), NMM (33.00 mg, 326.23 μmol, 35.87 μL, 5 eq) and HOAt (8.88 mg, 65.25 μmol, 1 eq). The mixture was stirred at 25° C. for 1 hr. The reaction mixture was extracted with EtOAc (3 mL*3). The combined organic layers were washed with brine (5 mL*2), dried over Na₂SO₄, filtered and concentrated under reduced pressure to give a residue. The residue was purified by prep-HPLC (column: Phenomenex luna C18 150*25 mm*10 um; mobile phase: [water (FA)-ACN]; gradient: 23%-53% B over 10 min). The eluent was concentrated and lyophilized to give I-648 (34 mg, 32.41 μmol, 49.67% yield, FA salt) as a white solid. LCMS (Method E): Rt=0.487 min, M+H=1003.8. SCF: Rt=3.321 min, ee %=99.6%. ¹H NMR (400 MHz, DMSO-d6) δ=12.59 (s, 1H), 8.44-8.32 (m, 1H), 8.29-8.23 (m, 1H), 8.14 (s, 1H), 7.99-7.94 (m, 1H), 7.91-7.80 (m, 2H), 7.51-7.34 (m, 4H), 7.26-7.21 (m, 2H), 4.89-4.66 (m, 1H), 4.49-4.37 (m, 1H), 4.36-4.22 (m, 3H), 4.03-3.80 (m, 2H), 3.73-3.35 (m, 10H), 3.28-2.56 (m, 12H), 2.23-2.14 (m, 1H), 2.01-1.56 (m, 15H), 1.52-1.49 (m, 1H), 1.52-1.38 (m, 2H), 1.14 (br s, 2H), 1.08-0.98 (m, 2H), 0.77-0.63 (m, 4H). ¹⁹F NMR (377 MHz, DMSO-d6) δ=−119.77 (br s, 1F), −120.11-−120.47 (m, 1F).

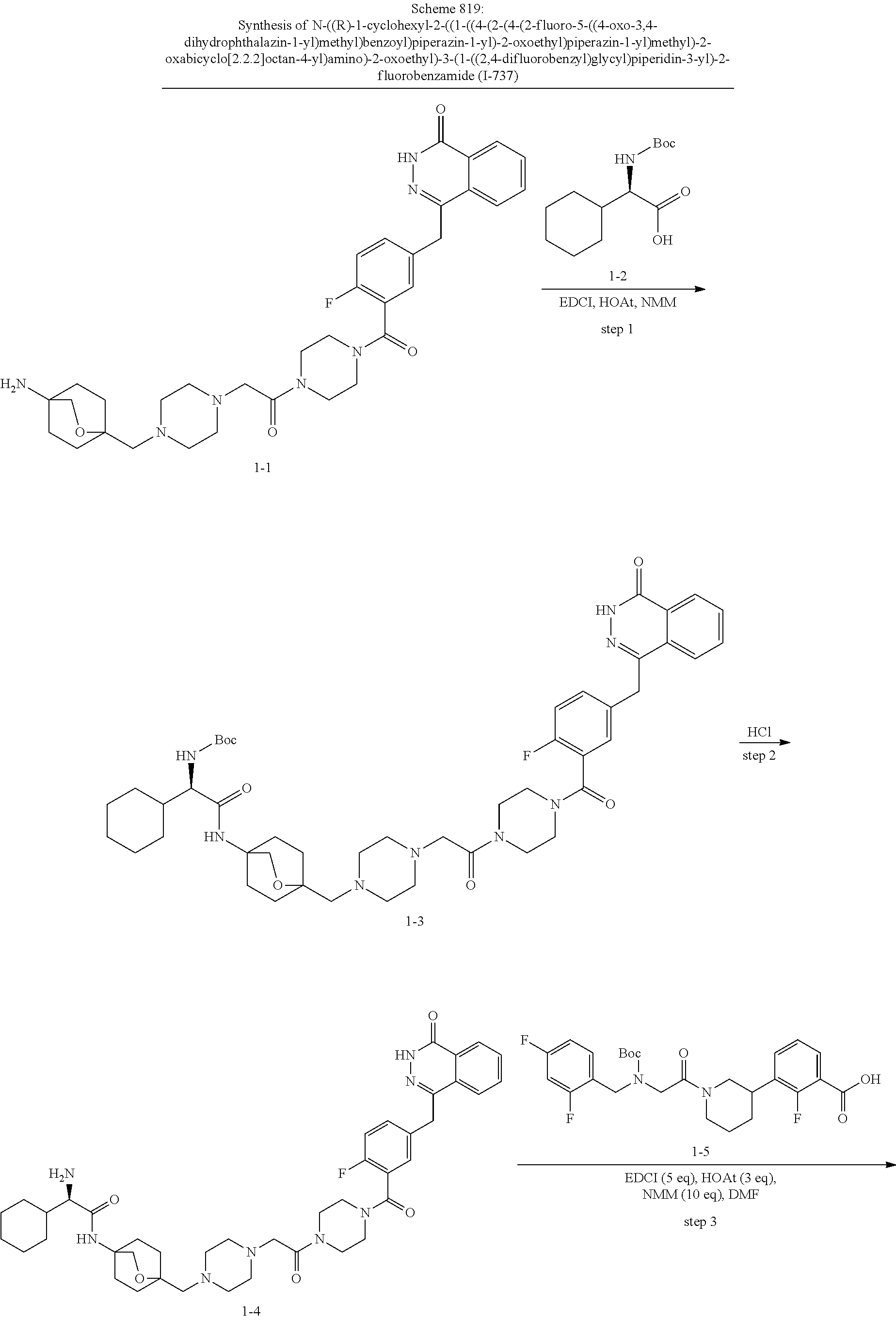

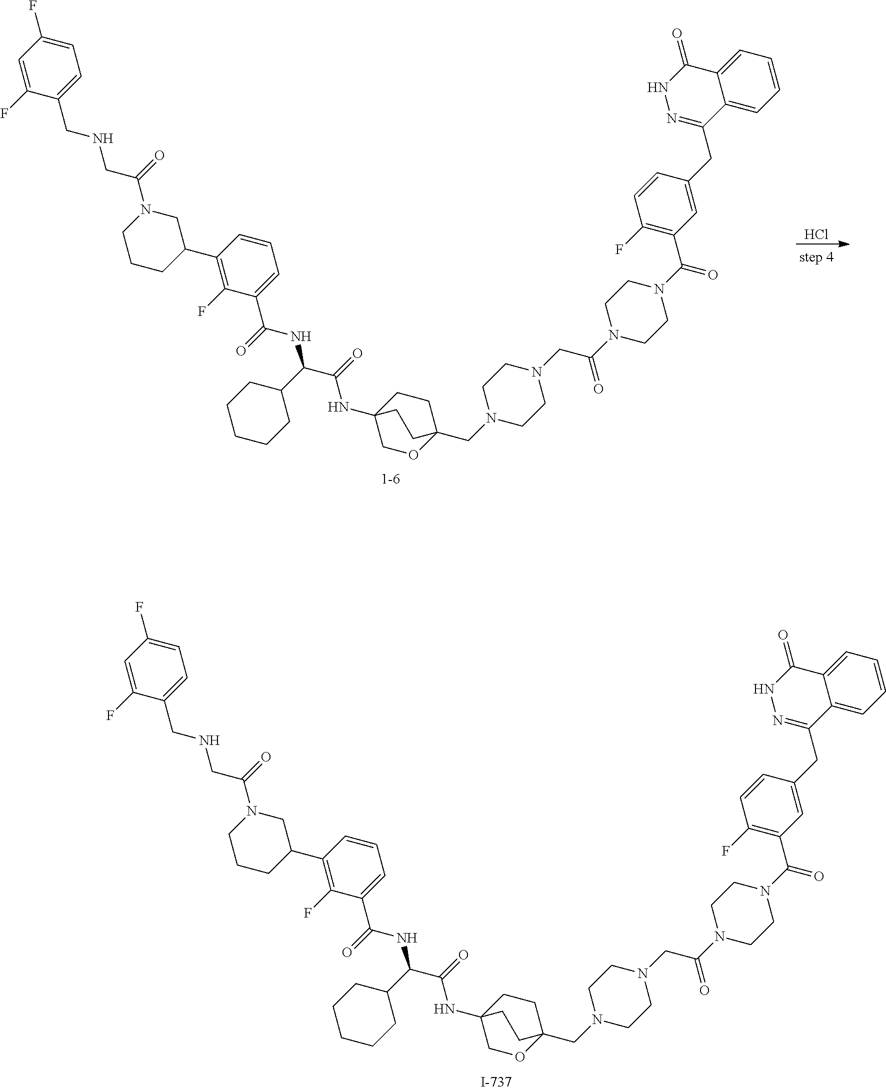 [SMILES: O=C(N[C@@H](C(=O)NC12CCC(CN3CCN(CC(=O)N4CCN(C(=O)c5cc(Cc6n[nH]c(=O)c7ccccc67)ccc5F)CC4)CC3)(CC1)OC2)C1CCCCC1)c1cccc(C2CCCN(C(=O)CNCc3ccc(F)cc3F)C2)c1F ; Cl ; O=C(N[C@@H](C(=O)NC12CCC(CN3CCN(CC(=O)N4CCN(C(=O)c5cc(Cc6n[nH]c(=O)c7ccccc67)ccc5F)CC4)CC3)(CC1)OC2)C1CCCCC1)c1cccc(C2CCCN(C(=O)CNCc3ccc(F)cc3F)C2)c1F]

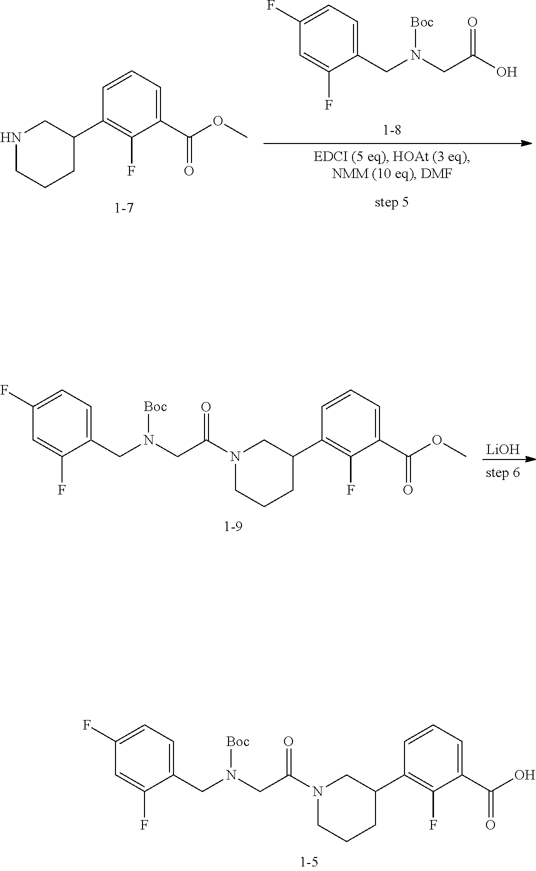

### Step 1: Synthesis of Intermediate 1-3

To a solution of Intermediate 1-1 (50 mg, 65.10 μmol, 1 eq, HCl salt) and Intermediate 1-2 (25.13 mg, 97.65 μmol, 1.5 eq) in DMF (0.5 mL) was added EDCI (62.40 mg, 325.50 μmol, 5 eq), HOAt (26.58 mg, 195.30 μmol, 27.32 μL, 3 eq) and NMM (65.85 mg, 651.00 μmol, 71.57 μL, 10 eq). The mixture was stirred at 25° C. for 1 hr. The reaction mixture was partitioned between EtOAc (5 mL) and water (5 mL) and then extracted with EtOAc (5 mL*2). The organic layers were dried over Na₂SO₄, filtered and concentrated under reduced pressure to give a residue. The residue was purified by flash silica gel chromatography (ISCO; 4 g SepaFlash Silica Flash Column, Eluent of 0-100% @Methanol: Dichloromethane 25 mL/min). Then the eluent was concentrated under reduced pressure to give Intermediate 1-3 (50 mg, 56.83 μmol, 87.29% yield, 99% purity) as white oil. LCMS (Method E): Rt=0.478 min, [M+H]⁺=871.5.

### Step 2: Synthesis of Intermediate 1-4

To a solution of Intermediate 1-3 (50 mg, 57.40 μmol, 1 eq) in DCM (0.5 mL) was added HCl/dioxane (2 M, 28.70 μL, 1.00 eq). The mixture was stirred at 25° C. for 0.5 hr. The reaction mixture was concentrated under reduced pressure to give Intermediate 1-4 (45 mg, 51.83 μmol, 90.30% yield, HCl salt) as a white solid. LCMS (Method G): Rt=0.545 min, [M+H]⁺=771.5.

### Step 3: Synthesis of Intermediate 1-6

To a solution of Intermediate 1-4 (45 mg, 55.73 μmol, 1 eq, HCl salt) and Intermediate 1-5 (42.35 mg, 83.60 μmol, 1.5 eq) in DMF (0.5 mL) was added NMM (56.37 mg, 557.35 μmol, 61.28 μL, 10 eq), EDCI (53.42 mg, 278.67 μmol, 5 eq) and HOAt (22.76 mg, 167.20 μmol, 23.39 μL, 3 eq). The mixture was stirred at 25° C. for 1 hr. The reaction mixture was partitioned between DCM (10 mL) and water (10 mL) and then extracted with DCM (10 mL*2). The organic layers were dried over Na₂SO₄, filtered and concentrated under reduced pressure to give a residue. The residue was purified by flash silica gel chromatography (ISCO; 12 g SepaFlash Silica Flash Column, Eluent of 0˜100% @Ethyl acetate:Methanol 60 mL/min). Then the product was concentrated under reduced pressure to give Intermediate 1-6 (60 mg, 40.49 μmol, 72.66% yield, 85% purity) as colorless oil. LCMS (Method E): Rt=0.554 min, [M+H]⁺=1259.9.

### Step 4: Synthesis of I-737

To a solution of Intermediate 1-6 (60 mg, 47.64 μmol, 1 eq) in DCM (0.5 mL) was added HCl/dioxane (2 M, 0.5 mL, 20.99 eq). The mixture was stirred at 25° C. for 0.5 hr. The reaction mixture was concentrated under reduced pressure to give a residue. The residue was purified by Prep-HPLC (column: Waters Xbridge 150*25 mm*5 um; mobile phase: [water (NH₄HCO₃)-ACN]; gradient: 33%-63% B over 10 min) and the eluent was concentrated to remove MeCN, and then lyophilized to afford I-737 (22.13 mg, 19.09 μmol, 40.07% yield, 100% purity) as a white solid. LCMS (Method G): Rt=0.684 min, [M+H]⁺=1159.7. SCF: Rt=2.276 min, 2.582 min. ¹H NMR (400 MHz, METHANOL-d4) δ=8.37 (d, J=7.6 Hz, 1H), 7.98-7.93 (m, 1H), 7.91-7.81 (m, 2H), 7.62-7.54 (m, 1H), 7.54-7.42 (m, 3H), 7.38 (br s, 1H), 7.30-7.22 (m, 1H), 7.20-7.13 (m, 1H), 7.00-6.90 (m, 2H), 4.62-4.54 (m, 1H), 4.39 (s, 2H), 4.37-4.30 (m, 1H), 4.03-3.93 (m, 2H), 3.93-3.78 (m, 4H), 3.76-3.63 (m, 3H), 3.59-3.47 (m, 4H), 3.39-3.34 (m, 2H), 3.28-2.98 (m, 5H), 2.85-2.68 (m, 1H), 2.63-2.45 (m, 7H), 2.30-2.22 (m, 2H), 2.20-2.09 (m, 2H), 2.05-1.56 (m, 16H), 1.36-1.03 (m, 5H). ¹⁹F NMR (376 MHz, METHANOL-d4) δ=−113.34 (s, 1F), −116.24 (br d, J=78.5 Hz, 1F), −120.77 (br d, J=20.2 Hz, 1F), −121.72 (br s, 1F).

### Step 5: Synthesis of Intermediate 1-9

To a solution of Intermediate 1-7 (300 mg, 1.10 mmol, 1 eq, HCl salt) and Intermediate 1-8 (495.30 mg, 1.64 mmol, 1.5 eq) in DMF (6 mL) was added EDCI (1.05 g, 5.48 mmol, 5 eq), HOAt (447.52 mg, 3.29 mmol, 459.94 μL, 3 eq) and NMM (1.11 g, 10.96 mmol, 1.20 mL, 10 eq). The mixture was stirred at 25° C. for 1 hr. The reaction mixture was partitioned between EtOAc (20 mL) and water (10 mL) and then extracted with EtOAc (10 mL*2). The organic layers were dried over Na₂SO₄, filtered and concentrated under reduced pressure to give a residue. The residue was purified by flash silica gel chromatography (ISCO; 40 g SepaFlash Silica Flash Column, Eluent of 0˜100% @Ethyl acetate:Methanol 60 mL/min). Then the eluent was concentrated under reduced pressure to give Intermediate 1-9 (300 mg, 564.80 μmol, 51.53% yield, 98% purity) as yellow oil. LCMS (Method E): Rt=0.635 min, [M+Na]⁺=543.2. SCF: Rt=1.421 min, 1.524 min.

### Step 6: Synthesis of Intermediate 1-5

To a solution of Intermediate 1-9 (300 mg, 576.33 μmol, 1 eq) in THF (1.5 mL), H₂O (1.5 mL) and MeOH (1.5 mL) was added LiOH·H₂O (72.55 mg, 1.73 mmol, 3 eq). The mixture was stirred at 25° C. for 1 hr. The reaction mixture was concentrated under reduced pressure to give Intermediate 1-5 (200 mg, 394.86 μmol, 68.51% yield) as a white solid. LCMS (Method E): Rt=0.549 min, [M+H]⁺=507.2. SCF: Rt=4.242 min, =5.117 min.

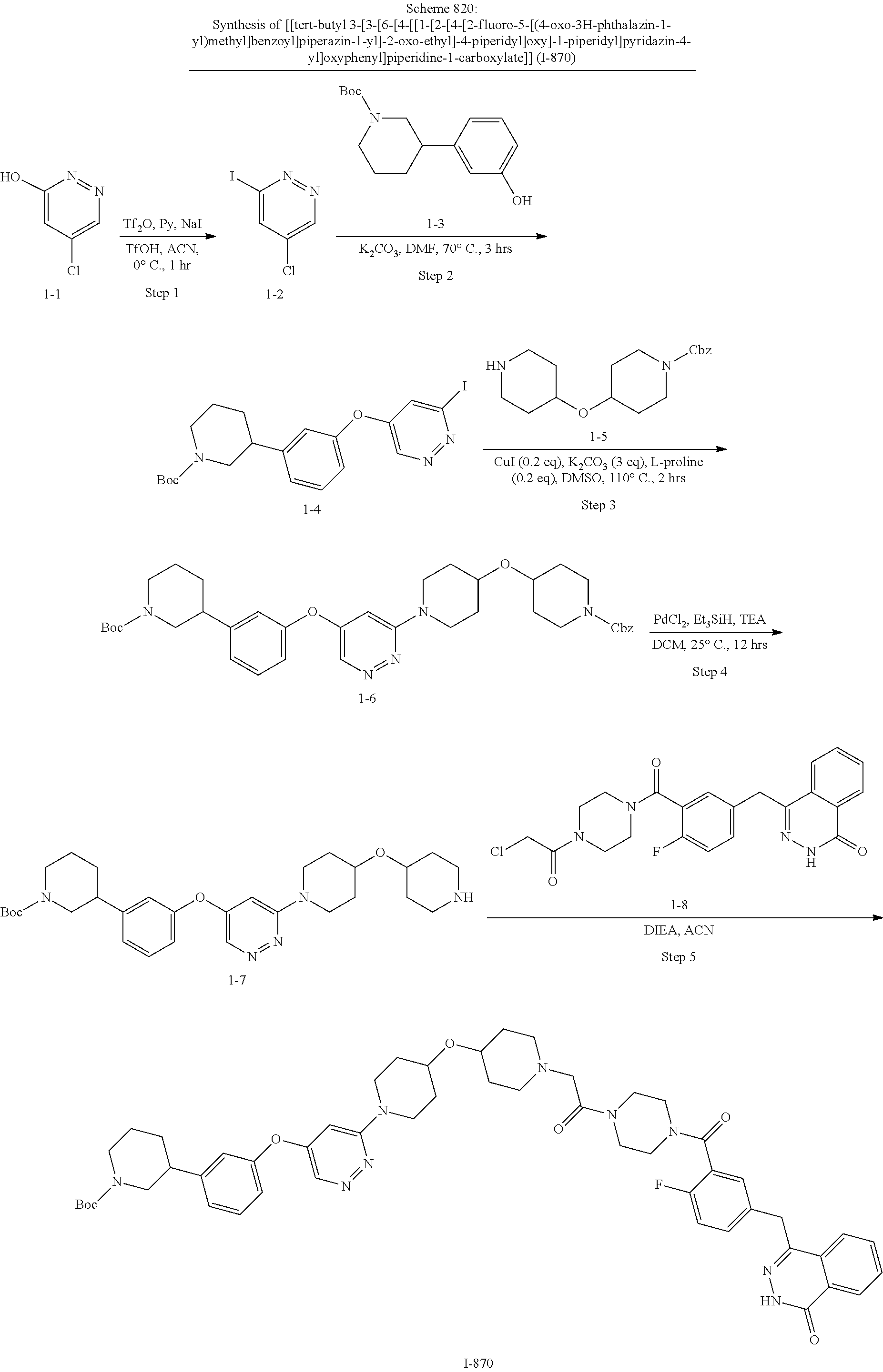

### Step 1: Synthesis of Intermediate 1-2

To a solution of intermediate 1-1 (10 g, 76.61 mmol, 1 eq) in ACN (100 mL) was added pyridine (7.27 g, 91.93 mmol, 7.42 mL, 1.2 eq) at 0° C., followed by Tf₂O (18.37 g, 65.12 mmol, 10.74 mL, 0.85 eq) dropwise. The mixture was stirred for 30 min, then charged with NaI (11.48 g, 76.61 mmol, 1 eq) in one portion. TfOH (12.65 g, 84.27 mmol, 7.46 mL, 1.1 eq) was added dropwise and the mixture was stirred for 1 h at 0° C. The pH of the reaction mixture was adjusted to 8-9 with aq. NaHCO₃ and extracted with EA (50 mL*3). The combined organic layer was dried over Na₂SO₄, filtered and concentrated in vacuo to give a residue. The residue was purified by flash silica gel chromatography (ISCO; 220 g SepaFlash Silica Flash Column, Eluent of 0-50% Ethyl acetate/Petroleum ether @100 mL/min) and the eluent was concentrated to give product. Intermediate 1-2 (6.5 g, 17.69 mmol, 23.10% yield, 65.449% purity) was obtained as a yellow solid. LCMS (Method D): Retention time=0.264 min, [M+H]⁺=240.7.

### Step 2: Synthesis of Intermediate 1-4

To a solution of intermediate 1-2 (6.5 g, 27.04 mmol, 1 eq) and intermediate 1-3 (7.50 g, 27.04 mmol, 1 eq) in DMF (65 mL) was added K₂CO₃ (7.47 g, 54.07 mmol, 2 eq). The mixture was stirred at 60° C. for 2 hr. The reaction mixture was diluted with water (60 mL) and extracted with DCM (60 mL*3), the combined organic phase was dried by Na₂SO₄, concentrated under reduced pressure to give a residue. The residue was purified by flash silica gel chromatography (ISCO; 220 g SepaFlash Silica Flash Column, Eluent of 0˜50% Ethyl acetate/Petroleum ether @100 mL/min) and the eluent was concentrated to give product. Intermediate 1-4 (10.5 g, 16.48 mmol, 60.98% yield, 75.567% purity) as a white solid. LCMS (Method D): Retention time: 0.487 min, [M+H]⁺=482.0. ¹H NMR (400 MHz, METHANOL-d4) δ=8.98 (d, J=2.8 Hz, 1H), 7.38 (d, J=2.8 Hz, 1H), 7.33-7.28 (m, 1H), 7.16 (s, 1H), 6.70 (br d, J=7.6 Hz, 1H), 6.65-6.56 (m, 1H), 4.14-4.04 (m, 4H), 2.60-2.49 (m, 1H), 2.05-1.96 (m, 2H), 1.78 (d, J=3.2 Hz, 2H), 1.45 (s, 9H).

### Step 3: Synthesis of Intermediate 1-6

To a solution of intermediate 1-4 (10.5 g, 21.81 mmol, 1.3 eq) and intermediate 1-5 (5.95 g, 16.78 mmol, 1 eq, HCl salt) in DMSO (105 mL) was added CuI (319.59 mg, 1.68 mmol, 0.1 eq), K₂CO₃ (6.96 g, 50.34 mmol, 3 eq) and (2S)-pyrrolidine-2-carboxylic acid (386.39 mg, 3.36 mmol, 0.2 eq). The mixture was stirred at 100° C. for 2 hr under N₂ atmosphere. The reaction mixture was filtered to give the filtrate. The filtrate was concentrated under reduced pressure to give the residue. The residue was purified by reversed phase HPLC (0.1% of FA) and the eluent was lyophilized. Intermediate 1-6 (1.6 g, 2.03 mmol, 85.415% purity) was obtained as a white solid. LCMS (Method D): Retention time: 0.454 min, [M+H]⁺=672.4.

### Step 4: Synthesis of Intermediate 1-7

To a solution of Intermediate 1-6 (1.58 g, 2.35 mmol, 1 eq) in DCM (16 mL) was added TEA (713.93 mg, 7.06 mmol, 982.02 μL, 3 eq) and Pd(OAc)₂ (264.00 mg, 1.18 mmol, 0.5 eq). The reaction mixture was degassed with N₂. Then Et₃SiH (3.28 g, 28.22 mmol, 4.51 mL, 12 eq) was added. The reaction mixture was stirred at 25° C. for 16 hr under N₂ atmosphere. The pH of the reaction mixture was adjusted to 8-9 with aq. NaHCO₃ and extracted with DCM (15 mL*3). The combined organic layer was dried over Na₂SO₄, filtered and concentrated in vacuo to give a residue. The residue was purified by reversed phase HPLC (0.1% of FA). The pH of the reaction mixture was adjusted to 8-9 with NH₃·H₂O and then lyophilized to afford the residue. Intermediate 1-7 (0.75 g, 1.35 mmol, 57.27% yield, 96.559% purity) was obtained as a white solid. LCMS (Method D): Retention time: 0.317 min, [M+H]⁺=538.4.

### Step 5: Synthesis of I-870

To a solution of intermediate 1-7 (0.7 g, 1.30 mmol, 1 eq) and intermediate 1-8 (576.55 mg, 1.30 mmol, 1 eq) in ACN (7 mL) was added DIEA (336.51 mg, 2.60 mmol, 453.52 μL, 2 eq). The mixture was stirred at 60° C. for 1 hr. The reaction mixture was diluted with water (7 mL) and extracted with DCM (7 mL*3). The combined organic phase was dried by Na₂SO₄, and concentrated under reduced pressure to give a residue. The crude product was purified by prep-HPLC (column: CD06-Waters Xbidge C18 150*40*10 um; mobile phase: [water(NH₄HCO₃)-ACN]; gradient: 43%-73% B over 10 min) and the eluent was concentrated to remove ACN and lyophilized to give product. I-870 (570 mg, 594.02 μmol, 45.63% yield, 98.388% purity) was obtained as a yellow solid. LCMS (Method H): Retention time=11.647 min, [M+H]⁺=944.6. ¹H NMR (400 MHz, METHANOL-d4) δ=8.40-8.31 (m, 1H), 8.26 (d, J=2.4 Hz, 1H), 8.01-7.91 (m, 1H), 7.90-7.79 (m, 2H), 7.53-7.41 (m, 2H), 7.40-7.34 (m, 1H), 7.23 (d, J=8.0 Hz, 1H), 7.16 (t, J=8.8 Hz, 1H), 7.10 (s, 1H), 7.06-6.99 (m, 1H), 6.49 (d, J=1.6 Hz, 1H), 4.38 (s, 2H), 4.14-4.00 (m, 2H), 3.92 (d, J=13.2 Hz, 2H), 3.81-3.64 (m, 5H), 3.59-3.48 (m, 3H), 3.30-3.12 (m, 6H), 2.94-2.66 (m, 5H), 2.33-2.19 (m, 2H), 2.05-1.97 (m, 1H), 1.89 (d, J=9.6 Hz, 4H), 1.79-1.69 (m, 2H), 1.69-1.47 (m, 6H), 1.45 (s, 9H).

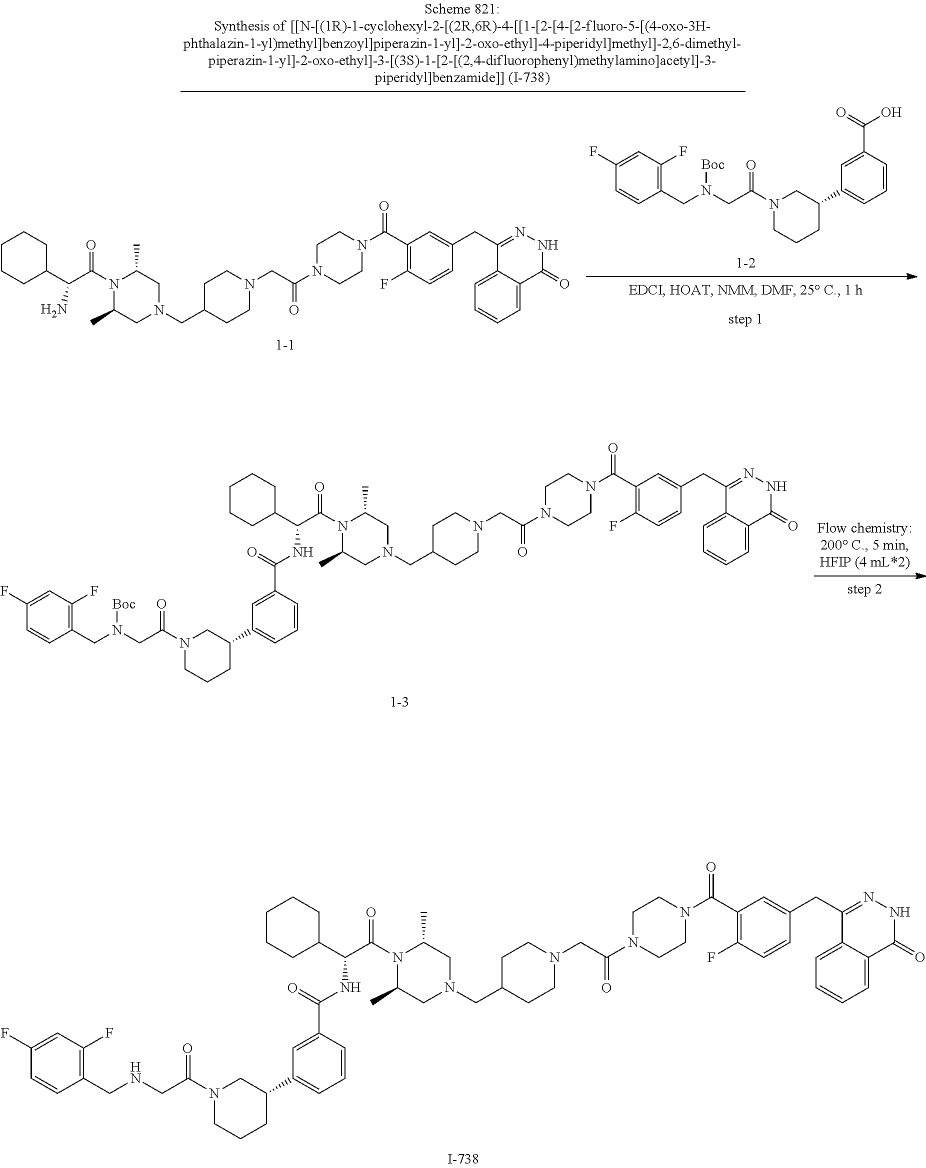

### Step 1: Synthesis of Intermediate 1-3

To a solution of Intermediate 1-2 (316.24 mg, 647.34 μmol, 1 eq) in DMF (5 mL) was added NMM (327.38 mg, 3.24 mmol, 355.85 μL, 5 eq), EDCI (372.28 mg, 1.94 mmol, 3 eq) and HOAT (88.11 mg, 647.34 μmol, 90.55 μL, 1 eq). The mixture was stirred at 25° C. for 15 min. Then Intermediate 1-1 (490 mg, 647.34 μmol, 1 eq) was added in and the mixture was stirred at 25° C. for 1 hr. The reaction was poured into H₂O (5 mL) and extracted with EA (10 mL*3). The combined organic layers were dried over Na₂SO₄, filtered and concentrated under reduced pressure to give a residue. The residue was purified by flash silica gel chromatography (ISCO; 40 g SepaFlash Silica Flash Column, Eluent of 0˜30% Dichloromethane:Methanol=4:1, gradient @40 mL/min) and concentrated under reduced pressure to give Intermediate 1-3 (600 mg, 444.82 μmol, 68.72% yield, 91% purity) as alight yellow solid. LCMS (Method D): Retention time: 0.400 min, [M+H]⁺=1227.7 SCF: Retention time: 1.017 min.

### Step 2: Synthesis of I-738

A solution of Intermediate 1-3 (1 eq, 0.2 g in HFIP (2 mL)) was pumped by pump 1 to flow reactor 1 (FLR1, SS, Coils reactor, 3.175(⅛″) mm, 10 mL, 200° C.). The residence time of flow reactor 1 was {FLR1}. The mixture was collected (100 mL). The reaction mixture was collected after running for 5 mins. The reaction was cooled to room temperature and concentrated under vacuum to give a crude product. The crude product was purified by reversed phase chromatography (0.1% FA) and lyophilized to give I-738 (68.11 mg, 56.71 μmol, 14.21% yield, 97.692% purity, FA salt) as a white solid. LCMS (Method D): Retention time: 0.314 min, [M+H]⁺=1127.6 SCF: Retention time: 1.674 min ¹H NMR (400 MHz, METHANOL-d4) δ=8.50 (s, 1H), 8.40-8.33 (m, 1H), 7.98-7.92 (m, 1H), 7.90-7.80 (m, 2H), 7.75 (s, 1H), 7.73-7.67 (m, 1H), 7.53-7.40 (m, 4H), 7.37 (t, J=6.0 Hz, 1H), 7.16 (t, J=8.8 Hz, 1H), 7.03-6.91 (m, 2H), 4.79-4.74 (m, 1H), 4.59-4.50 (m, 1H), 4.38 (s, 2H), 3.92 (d, J=13.2 Hz, 2H), 3.72 (s, 4H), 3.71-3.55 (m, 6H), 3.52 (d, J=4.4 Hz, 1H), 3.48 (d, J=5.2 Hz, 1H), 3.33 (s, 1H), 3.22-3.09 (m, 3H), 2.88-2.68 (m, 4H), 2.62-2.41 (m, 4H), 2.39-2.27 (m, 2H), 2.07-1.93 (m, 2H), 1.91-1.73 (m, 8H), 1.72-1.54 (m, 4H), 1.45 (d, J=5.6 Hz, 6H), 1.42-1.14 (m, 6H), 1.06 (d, J=12.0 Hz, 2H). ¹⁹F NMR (377 MHz, METHANOL-d4) δ=−112.21, −115.45, −120.69.

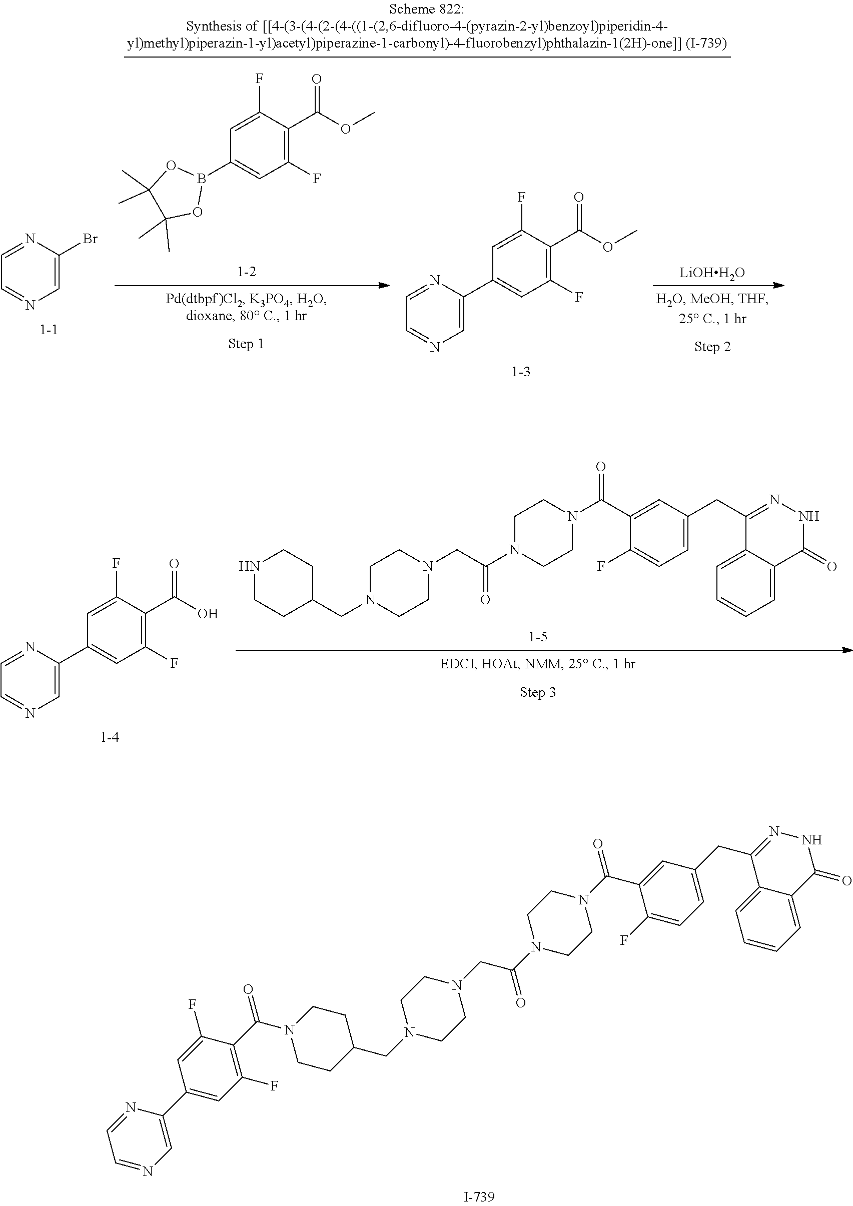

### Step 1: Synthesis of Intermediate 1-3

To a solution of Intermediate 1-1 (300 mg, 1.89 mmol, 1 eq) in H₂O (1.2 mL) and 1,4-dioxane (6 mL) was added Intermediate 1-2 (562.49 mg, 1.89 mmol, 1 eq), Pd(dtbpf)Cl₂ (122.98 mg, 188.70 μmol, 0.1 eq) and K₃PO₄ (1.20 g, 5.66 mmol, 3 eq). The mixture was stirred at 80° C. for 1 hr under N₂. The reaction mixture was diluted with H₂O (5 mL) and extracted with EA (5 mL*2). The combined organic layers were dried over Na₂SO₄, filtered and concentrated under reduced pressure to give a residue. The residue was purified by flash silica gel chromatography (ISCO; 20 g SepaFlash Silica Flash Column, Eluent of 0˜18% Ethyl acetate/Petroleum ether gradient @40 mL/min). The eluent was concentrated under reduced pressure to give the product. Intermediate 1-3 (450 mg, 1.73 mmol, 91.77% yield, 96.280% purity) was obtained as a yellow solid. LCMS (Method D): Rt=0.356 min, [M+H]⁺=250.9.

### Step 2: Synthesis of Intermediate 1-4

To a solution of Intermediate 1-3 (400 mg, 1.60 mmol, 1 eq) in H₂O (1.5 mL), MeOH (1.5 mL) and THF (1.5 mL) was added LiOH H₂O (201.26 mg, 4.80 mmol, 3 eq). The mixture was stirred at 25° C. for 1 hr. Intermediate 1-4 (350 mg, 1.31 mmol, 81.96% yield, 88.419% purity) was obtained as a yellow solid and was used in the next step without further purification. LCMS (Method D): Rt=0.267 min, [M+H]⁺=237.0.

### Step 3: Synthesis of I-739

To a solution of Intermediate 1-4 (200 mg, 846.83 μmol, 1 eq) and Intermediate 1-5 (530.26 mg, 846.83 μmol, 1 eq, HCl salt) in DMF (5 mL) was added HOAt (115.26 mg, 846.83 μmol, 118.46 μL, 1 eq), EDCI (487.02 mg, 2.54 mmol, 3 eq) and NMM (428.27 mg, 4.23 mmol, 465.51 μL, 5 eq). The mixture was stirred at 25° C. for 0.5 hr. The reaction mixture was diluted with H₂O (10 mL) and extracted with EA (10 mL*3). The combined organic layers were washed with brine (10 mL*3), dried over Na₂SO₄, filtered and concentrated under reduced pressure to give a residue. The crude product was purified by reversed-phase chromatography (0.1% NH₃·H₂O), and the eluent was concentrated and lyophilized to give the desired product. I-739 (176.13 mg, 211.94 μmol, 25.03% yield, 97.21% purity) was obtained as a white solid. LCMS (Method F): Rt=0.575 min, [M+H]⁺=808.4. ¹H NMR (400 MHz, METHANOL-d4) δ=9.21 (d, J=1.2 Hz, 1H), 8.75-8.69 (m, 1H), 8.62 (d, J=2.4 Hz, 1H), 8.39-8.34 (m, 1H), 7.97-7.79 (m, 5H), 7.51-7.34 (m, 2H), 7.21-7.11 (m, 1H), 4.69 (d, J=12.8 Hz, 1H), 4.38 (s, 2H), 3.84-3.64 (m, 4H), 3.63-3.46 (m, 3H), 3.34 (d, J=6.4 Hz, 1H), 3.30-3.11 (m, 4H), 2.97-2.87 (m, 1H), 2.51 (d, J=1.2 Hz, 8H), 2.29-2.18 (m, 2H), 1.97-1.77 (m, 3H), 1.27-1.10 (m, 2H). ¹⁹F NMR (376 MHz, METHANOL-d4) δ=−114.395, −120.727.

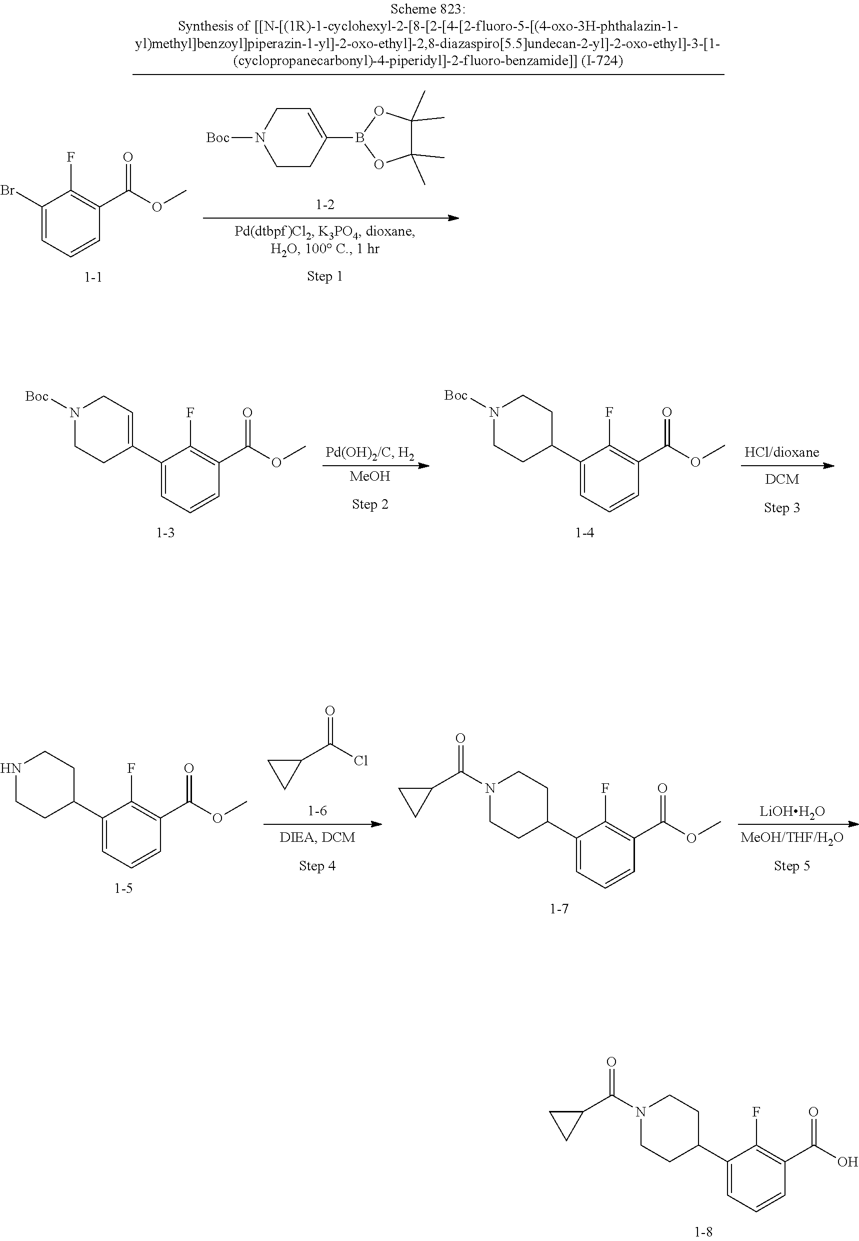

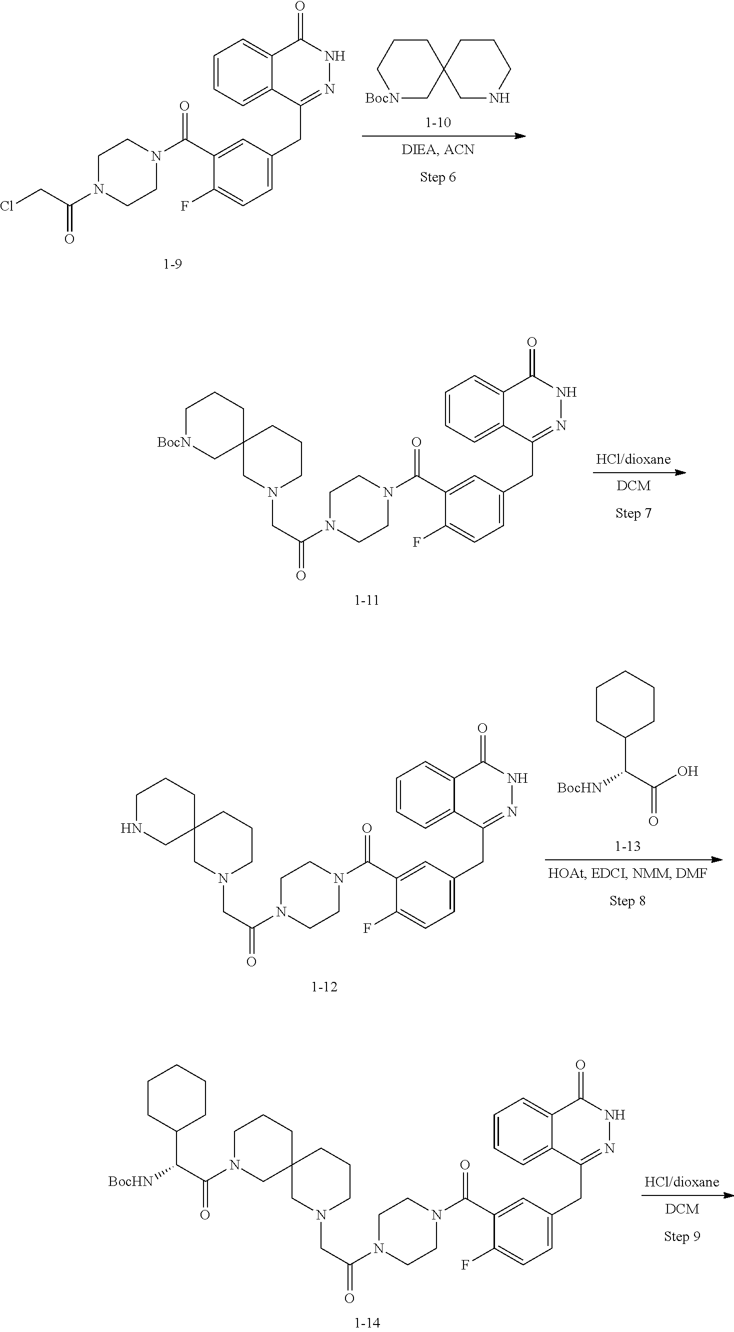 [SMILES: O=C(CCl)N1CCN(C(=O)c2cc(Cc3n[nH]c(=O)c4ccccc34)ccc2F)CC1 ; CC(C)(C)OC(=O)N1CCCC2(CCCNC2)C1 ; CC(C)(C)OC(=O)N1CCCC2(CCCN(CC(=O)N3CCN(C(=O)c4cc(Cc5n[nH]c(=O)c6ccccc56)ccc4F)CC3)C2)C1 ; O=C(CN1CCCC2(CCCNC2)C1)N1CCN(C(=O)c2cc(Cc3n[nH]c(=O)c4ccccc34)ccc2F)CC1 ; CC(C)(C)OC(=O)N[C@@H](C(=O)O)C1CCCCC1 ; CC(C)(C)OC(=O)N[C@@H](C(=O)N1CCCC2(CCCN(CC(=O)N3CCN(C(=O)c4cc(Cc5n[nH]c(=O)c6ccccc56)ccc4F)CC3)C2)C1)C1CCCCC1]

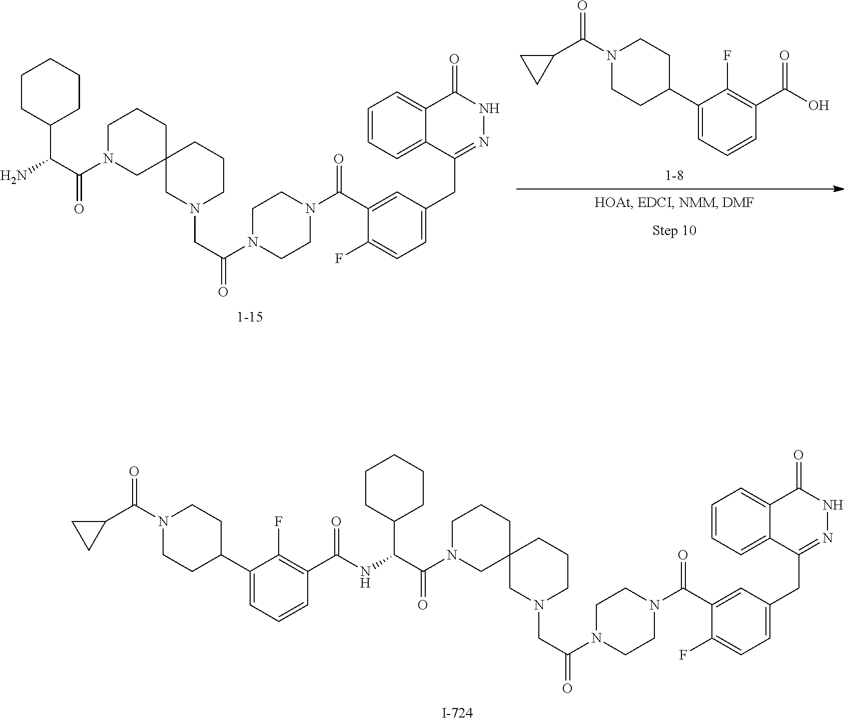 [SMILES: N[C@@H](C(=O)N1CCCC2(CCCN(CC(=O)N3CCN(C(=O)c4cc(Cc5n[nH]c(=O)c6ccccc56)ccc4F)CC3)C2)C1)C1CCCCC1 ; O=C(O)c1cccc(C2CCN(C(=O)C3CC3)CC2)c1F ; O=C(N[C@@H](C(=O)N1CCCC2(CCCN(CC(=O)N3CCN(C(=O)c4cc(Cc5n[nH]c(=O)c6ccccc56)ccc4F)CC3)C2)C1)C1CCCCC1)c1cccc(C2CCN(C(=O)C3CC3)CC2)c1F]

### Step 1: Synthesis of Intermediate 1-3

To a solution of Intermediate 1-2 (796.13 mg, 2.57 mmol, 1 eq) in dioxane (6 mL) and H₂O (1.2 mL) was added Intermediate 1-1 (600 mg, 2.57 mmol, 1 eq), K₃PO₄ (1.64 g, 7.72 mmol, 3 eq) and Pd(dtbpf)Cl₂ (167.81 mg, 257.47 μmol, 0.1 eq). The mixture was stirred at 100° C. for 1 hr under N₂. The reaction mixture diluted with H₂O (5 mL) and extracted with EA (5 mL*2), The combined organic layers were dried over Na₂SO₄, filtered and concentrated under reduced pressure to give a residue. The residue was purified by flash silica gel chromatography (ISCO; 20 g SepaFlash Silica Flash Column, Eluent of 0˜10% Ethyl acetate/Petroleum ether gradient @60 mL/min). The eluent was concentrated under reduced pressure to give Intermediate 1-3 (940 mg, crude) as yellow oil. LCMS (Method D): Rt=0.481 min, [M+Na]⁺=358.1. ¹H NMR (400 MHz, METHANOL-d4) δ=7.82-7.76 (m, 1H), 7.55-7.49 (m, 1H), 7.26-7.20 (m, 1H), 5.94 (s, 1H), 4.07 (s, 2H), 3.90 (s, 3H), 3.69-3.56 (m, 2H), 2.55-2.42 (m, 2H), 1.50 (s, 9H)¹⁹F NMR (377 MHz, METHANOL-d4) δ=−115.233.

### Step 2: Synthesis of Intermediate 1-4

To a solution of Intermediate 1-3 (940 mg, 2.80 mmol, 1 eq) in MeOH (10 mL) was added Pd(OH)₂/C (500 mg, 20% purity). The flask was degassed and purged with H₂ 3 times, then the mixture was stirred at 25° C. for 1 hr under H₂ (15 psi) atmosphere. The reaction was filtered and the filter cake was washed with MeOH (3 mL*3). The filtrate was concentrated under reduced pressure to give Intermediate 1-4 (900 mg, 2.48 mmol, 88.32% yield, 92.8% purity) as a yellow oil. LCMS (Method D): Rt=0.481 min, [M+Na]⁺=360.1.

### Step 3: Synthesis of Intermediate 1-5

A solution of Intermediate 1-4 (800 mg, 2.37 mmol, 1 eq) in DCM (4 mL) and HCl/dioxane (2 M, 8 mL) was stirred at 25° C. for 1 hr. The mixture was concentrated under reduced pressure to give Intermediate 1-5 (650 mg, crude, HCl salt) as a yellow solid. LCMS (Method D): Rt=0.224 min, [M+H]⁺=238.1. ¹H NMR (400 MHz, METHANOL-d4) δ=7.85-7.78 (m, 1H), 7.60-7.52 (m, 1H), 7.32-7.23 (m, 1H), 3.91 (s, 3H), 3.56-3.48 (m, 2H), 3.30-3.26 (m, 1H), 3.24-3.14 (m, 2H), 2.11-1.97 (m, 4H).

### Step 4: Synthesis of Intermediate 1-7

To a solution of Intermediate 1-5 (650 mg, 2.37 mmol, 1 eq, HCl salt) in DCM (8 mL) was added Intermediate 1-6 (297.87 mg, 2.85 mmol, 258.57 μL, 1.2 eq) and DIEA (920.70 mg, 7.12 mmol, 1.24 mL, 3 eq). The mixture was stirred at 0° C. for 0.5 hr. The mixture was diluted with H₂O (10 mL) and extracted with CH₂Cl₂ (10 mL*1). The combined organic layers were dried over Na₂SO₄, filtered and concentrated under reduced pressure to give a residue. The residue was purified by flash silica gel chromatography (ISCO; 20 g SepaFlash Silica Flash Column, Eluent of 0˜35% Ethyl acetate/Petroleum ether gradient @60 mL/min). The eluent was concentrated under reduced pressure to give Intermediate 1-7 (650 mg, crude, HCl salt) (570 mg, 1.84 mmol, 77.36% yield, 98.4% purity) as colorless oil. LCMS (Method D): Rt=0.405 min, [M+H]⁺=306.1.

### Step 5: Synthesis of Intermediate 1-8

To a solution of Intermediate 1-7 (470 mg, 1.54 mmol, 1 eq) in MeOH (1.5 mL), THF (1.5 mL) and H₂O (1.5 mL) was added LiOH H₂O (193.76 mg, 4.62 mmol, 3 eq). The mixture was stirred at 25° C. for 1 hr. The mixture was diluted with H₂O (3 mL) and HCl (1M) was added to adjust the pH to 4. The mixture was extracted with EA (3 mL*2). The combined organic layers were dried over Na₂SO₄, filtered and concentrated under reduced pressure to give Intermediate 1-8 (550 mg, crude) as yellow oil. LCMS (Method D): Rt=0.343 min, [M+H]⁺=292.1. ¹H NMR (400 MHz, METHANOL-d4) δ=7.80-7.74 (m, 1H), 7.55-7.49 (m, 1H), 7.25-7.18 (m, 1H), 4.74-4.62 (m, 1H), 4.55-4.42 (m, 1H), 3.30-3.22 (m, 2H), 2.85-2.72 (m, 1H), 2.01-1.92 (m, 2H), 1.91-1.83 (m, 1H), 1.81-1.60 (m, 2H), 0.92-0.80 (m, 4H). ¹⁹F NMR (377 MHz, METHANOL-d4) δ=−119.038.

### Step 6: Synthesis of Intermediate 1-11

To a solution of Intermediate 1-9 (700 mg, 1.58 mmol, 1 eq) in ACN (7 mL) was added Intermediate 1-10 (402.05 mg, 1.58 mmol, 1 eq) and DIEA (612.84 mg, 4.74 mmol, 825.93 μL, 3 eq). The mixture was stirred at 40° C. for 1 hr. The mixture was diluted with H₂O (10 mL) and extracted with EA (10 mL*2). The combined organic layers were dried over Na₂SO₄, filtered and concentrated under reduced pressure to give a residue. The residue was purified by flash silica gel chromatography (ISCO; 40 g SepaFlash Silica Flash Column, Eluent of 0-100% Ethyl acetate/Petroleum ether@60 mL/min). The eluent was concentrated under reduced pressure to give Intermediate 1-11 (950 mg, 1.43 mmol, 90.19% yield, 99.15% purity) as a yellow solid. LCMS (Method D): Rt=0.345 min, [M+H]⁺=661.2. ¹H NMR (400 MHz, METHANOL-d4) δ=8.41-8.33 (m, 1H), 8.00-7.92 (m, 1H), 7.91-7.80 (m, 2H), 7.53-7.33 (m, 2H), 7.21-7.11 (m, 1H), 4.43-4.33 (m, 2H), 4.02-3.49 (m, 8H), 3.01-2.81 (m, 1H), 2.68-2.52 (m, 1H), 2.29-2.04 (m, 2H), 1.67-1.40 (m, 11H), 1.29 (s, 5H), 1.17-1.04 (m, 1H). SCF: Rt=4.028 min, 5.423 min, ¹⁹F NMR (377 MHz, METHANOL-d4) δ=−120.802.

### Step 7: Synthesis of Intermediate 1-12

A solution of Intermediate 1-11 (100 mg, 151.34 μmol, 1 eq) in DCM (0.5 mL) and HCl/dioxane (2 M, 0.5 mL) was stirred at 25° C. for 1 hr. The mixture was concentrated under reduced pressure to give Intermediate 1-12 (95 mg, crude, HCl salt) as a white solid. LCMS (Method D): Rt=0.232 min, [M+H]⁺=561.2.

### Step 8: Synthesis of Intermediate 1-14

To a solution of Intermediate 1-13 (40.94 mg, 159.10 μmol, 1 eq) in DMF (1 mL) was added EDCI (91.50 mg, 477.29 μmol, 3 eq), HOAt (21.65 mg, 159.10 μmol, 22.26 μL, 1 eq), Intermediate 1-12 (95 mg, 159.10 μmol, 1 eq, HCl salt) and NMM (80.46 mg, 795.48 μmol, 87.46 μL, 5 eq). The mixture was stirred at 25° C. for 1 hr. The mixture was diluted with H₂O (1 mL) and extracted with EA (1.5 mL*2). The combined organic layers were washed with brine (1 mL*2), dried over Na₂SO₄, filtered and concentrated under reduced pressure to give a residue. The residue was purified by flash silica gel chromatography (ISCO; 12 g SepaFlash Silica Flash Column, Eluent of 0˜10% MeOH/Ethyl acetate gradient @60 mL/min). The eluent was concentrated under reduced pressure to give Intermediate 1-14 (100 mg, 124.13 μmol, 78.02% yield, 99.3% purity) as yellow gum. LCMS (Method D): Rt=0.400 min, [M+H]⁺=800.4. SCF: Rt=4.251 min, 5.739 min.

### Step 9: Synthesis of Intermediate 1-15

To a solution of Intermediate 1-14 (100 mg, 125.00 μmol, 1 eq) in DCM (0.5 mL) and HCl/dioxane (2 M, 0.5 mL) was stirred at 25° C. for 1 hr. The mixture was concentrated under reduced pressure to give Intermediate 1-15 (95 mg, crude, HCl salt) as a white solid. LCMS (Method D): Rt=0.270 min, [M+H]⁺=700.3.

### Step 10: Synthesis of I-724

To a solution of Intermediate 1-8 (41.34 mg, 141.92 μmol, 1.1 eq) in DMF (1 mL) was added EDCI (74.20 mg, 387.06 μmol, 3 eq), HOAt (17.56 mg, 129.02 μmol, 18.05 μL, 1 eq), Intermediate 1-15 (95 mg, 129.02 μmol, 1 eq, HCl salt) and NMM (65.25 mg, 645.10 μmol, 70.92 μL, 5 eq). The mixture was stirred at 25° C. for 1 hr. The reaction mixture was purified by reversed-phase chromatography (0.1% FA condition). The eluent was concentrated under reduced pressure to remove ACN and then lyophilized to give I-724 (35.31 mg, 36.28 μmol, 28.12% yield, 100% purity) as a white solid. LCMS (Method D): Rt=0.434 min, [M+H]⁺=973.7. ¹H NMR (400 MHz, METHANOL-d4) δ=8.42-8.26 (m, 1H), 8.01-7.74 (m, 3H), 7.63-6.99 (m, 6H), 5.01-4.91 (m, 1H), 4.71-4.58 (m, 1H), 4.52-4.31 (m, 3H), 4.13-3.34 (m, 11H), 3.29-3.08 (m, 5H), 2.85-2.06 (m, 5H), 2.05-1.41 (m, 18H), 1.36-1.05 (m, 6H), 0.96-0.72 (m, 4H). SCF: Rt=1.536 min, 1.960 min. ¹⁹F NMR (377 MHz, METHANOL-d4) δ=−120.501, −122.108.

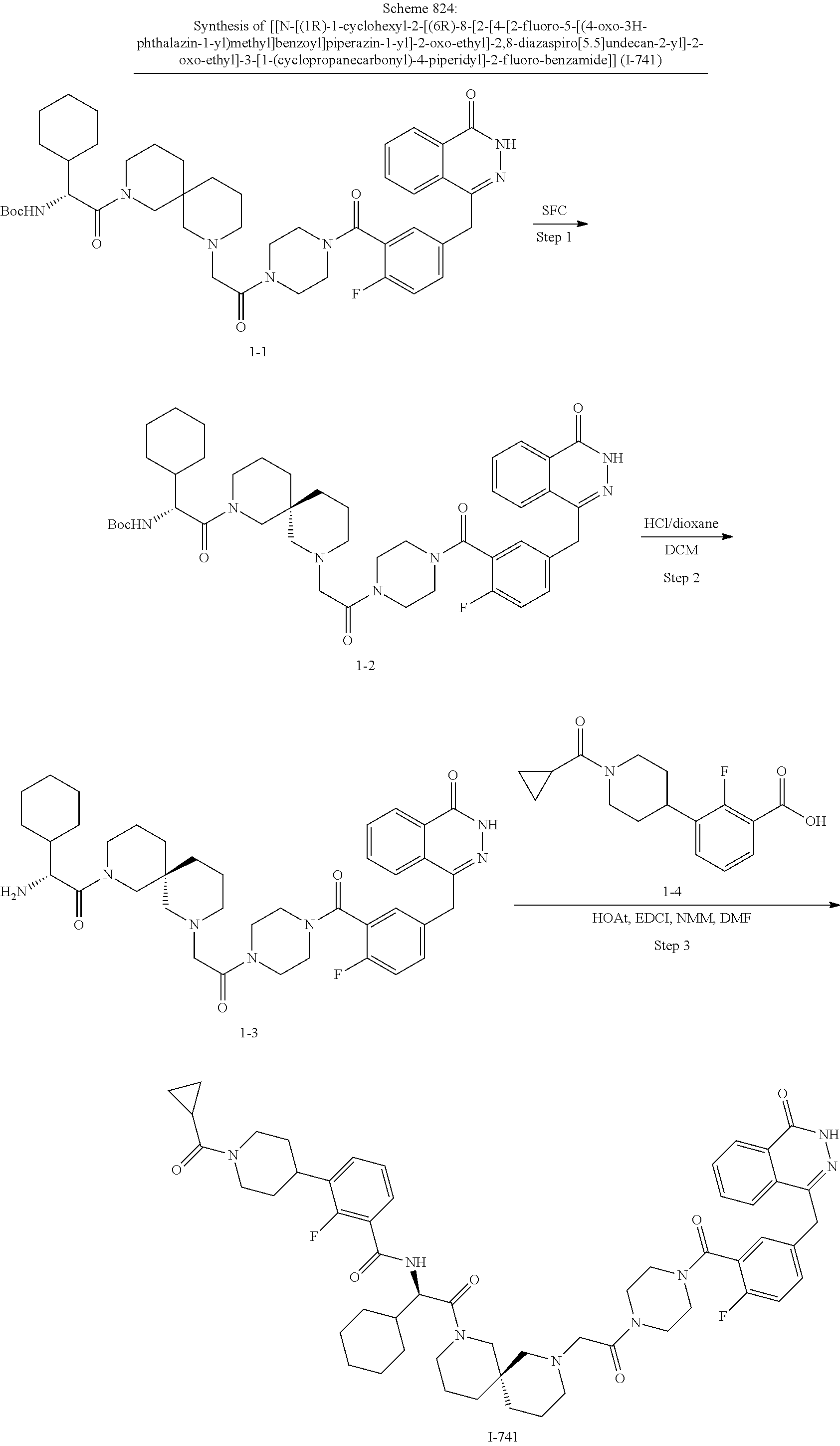

### Step 1: Synthesis of Intermediate 1-2

Intermediate 1-1 (200 mg, 250.01 mol) was separated by chiral SFC (column: ChiralPak IH, 250*50 mm, 10 um; mobile phase: [Hexane-EtOH: CAN (4:1) (0.1 IPAM)]; B %: 35%, isocratic elution mode). The eluent was concentrated under reduced pressure to give Intermediate 1-3 (85 mg, 106.25 mol, 42.50% yield, 100% purity) as a yellow solid. LCMS (Method D): Rt=0.400 min, [M+H]⁺=800.4. SFC: Rt=4.198 min.

### Step 2: Synthesis of Intermediate 1-3

To a solution of Intermediate 1-3 (85 mg, 106.25 μmol, 1 eq) in DCM (0.5 mL) and HCl/dioxane (2 M, 1 mL) was stirred at 25° C. for 1 hr. The mixture was concentrated under reduced pressure to give Intermediate 1-3 (80 mg, crude, HCl salt) as a white solid. LCMS (Method D): Rt=0.280 min, [M+H]⁺=700.3.

### Step 3: Synthesis of I-741

To a solution of Intermediate 1-4 (19.78 mg, 67.91 μmol, 1 eq) in DMF (1 mL) was added EDCI (39.05 mg, 203.72 μmol, 3 eq), HOAt (9.24 mg, 67.91 μmol, 9.50 μL, 1 eq), Intermediate 1-3 (50 mg, 67.91 μmol, 1 eq, HCl salt) and NMM (34.34 mg, 339.53 μmol, 37.33 μL, 5 eq). The mixture was stirred at 25° C. for 1.5 hr. The mixture was filtered and the filtrate was purified by reversed-phase chromatography (0.1% FA condition). The eluent was concentrated under reduced pressure to remove ACN and then lyophilized to give I-741 (30 mg, 26.80 μmol, 39.46% yield, 97.104% purity, TFA salt) as a white solid. LCMS (Method D): Rt=0.409 min, [M+H]⁺=973.5. ¹H NMR (400 MHz, METHANOL-d4) δ=8.38-8.33 (m, 1H), 8.02-7.75 (m, 3H), 7.65-7.07 (m, 6H), 5.02-4.90 (m, 1H), 4.73-4.59 (m, 1H), 4.54-4.27 (m, 3H), 4.16-3.34 (m, 11H), 3.30-3.00 (m, 5H), 2.86-2.08 (m, 5H), 2.03-1.42 (m, 18H), 1.38-1.05 (m, 6H), 0.92-0.77 (m, 4H). SCF: Rt=1.529 min. ¹⁹F NMR (377 MHz, METHANOL-d4) δ=−76.947, −120.479, −121.548.

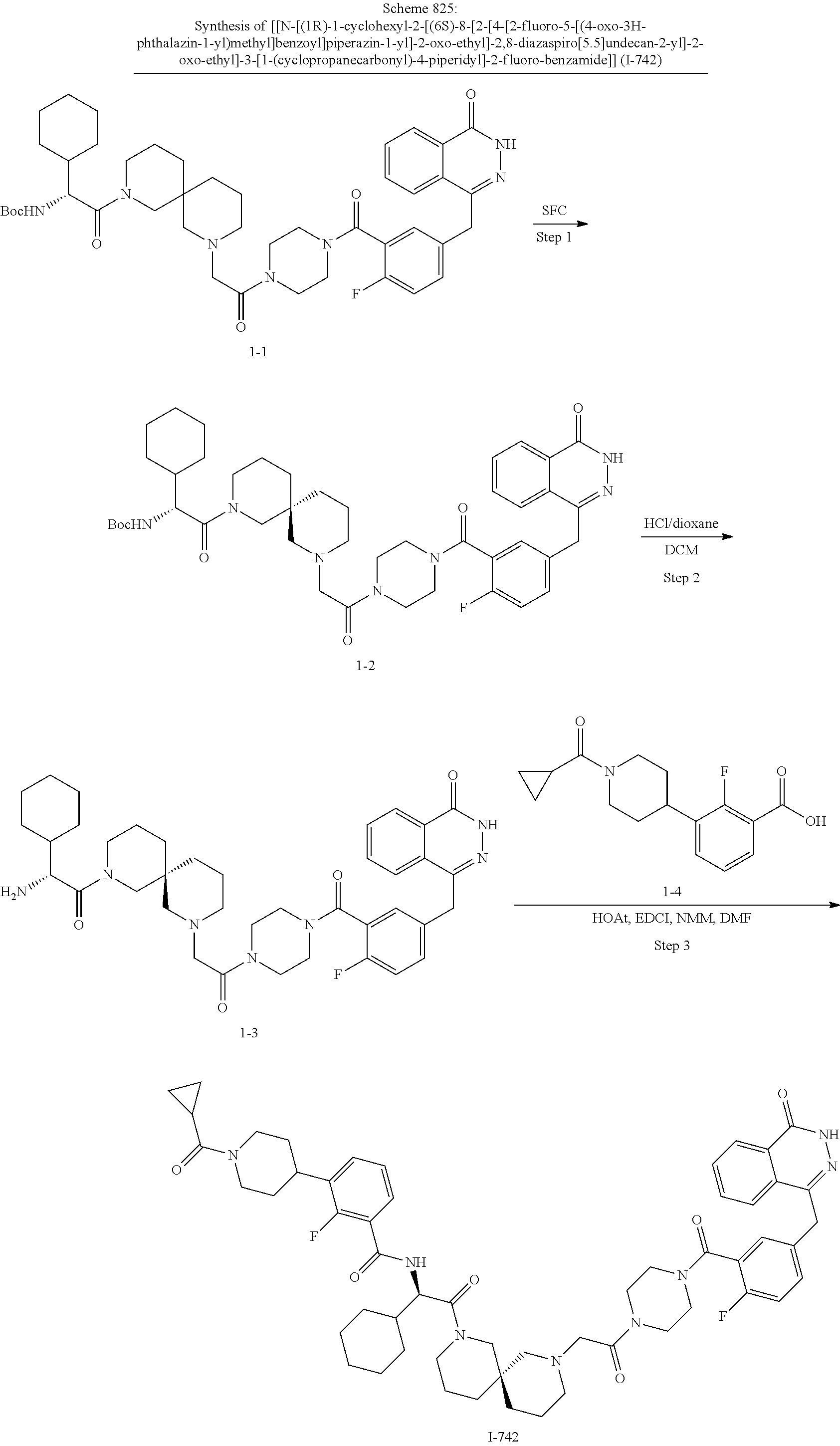

### Step 1: Synthesis of Intermediate 1-2

Intermediate 1-1 (200 mg, 250.01 μmol, 1 eq) was purified by SFC separation (column: ChiralPak IH, 250*50 mm, 10 um; mobile phase: [Hexane-EtOH: ACN (4:1) (0.1IPAM)]; B %: 35%, isocratic elution mode). The eluent was concentrated under reduced pressure to give intermediate 1-2 (90 mg, 111.24 mol, 44.49% yield, 98.874% purity) as a yellow solid. LCMS (Method D): Rt=0.397 min, [M+H]⁺=800.4. SCF: Rt=5.672 min.

### Step 2: Synthesis of Intermediate 1-3

A solution of intermediate 1-2 (90 mg, 112.50 μmol, 1 eq) in DCM (0.5 mL) and HCl/dioxane (2 M, 2 mL) was stirred at 25° C. for 1 hr. The mixture was concentrated under reduced pressure to give product. Intermediate 1-3 (85 mg, crude, HCl salt) was obtained as a white solid. LCMS (Method D): Rt=0.281 min, [M+H]⁺=700.3.

### Step 3: Synthesis of I-742

To a solution of Intermediate 1-4 (19.78 mg, 67.91 μmol, 1 eq) in DMF (1 mL) was added EDCI (39.05 mg, 203.72 μmol, 3 eq), HOAt (9.24 mg, 67.91 μmol, 9.50 μL, 1 eq), intermediate 1-3 (50 mg, 67.91 μmol, 1 eq, HCl salt) and NMM (34.34 mg, 339.53 μmol, 37.33 μL, 5 eq). The mixture was stirred at 25° C. for 1.5 hrs. The mixture was purified by reversed-phase chromatography (0.10% FA condition). The eluent was concentrated under reduced pressure to remove ACN and then lyophilized to give I-742 (26.62 mg, 27.11 μmol, 39.92% yield, 99.094% purity) as a white solid. LCMS (Method D): Rt=0.418 min, [M+H]⁺=973.8. ¹H NMR (400 MHz, METHANOL-d4) δ=8.39-8.31 (m, 1H), 7.97-7.91 (m, 1H), 7.90-7.79 (m, 2H), 7.60-7.30 (m, 4H), 7.27-7.09 (m, 2H), 5.07-4.88 (m, 1H), 4.72-4.56 (m, 1H), 4.53-4.41 (m, 1H), 4.41-4.30 (m, 2H), 4.14-3.32 (m, 11H), 3.29-3.08 (m, 5H), 2.83-2.05 (m, 5H), 2.01-1.42 (m, 18H), 1.36-1.06 (m, 6H), 0.93-0.75 (m, 4H). ¹⁹F NMR (377 MHz, METHANOL-d4) δ=−120.622, −122.310. SCF: Rt=1.944 min.

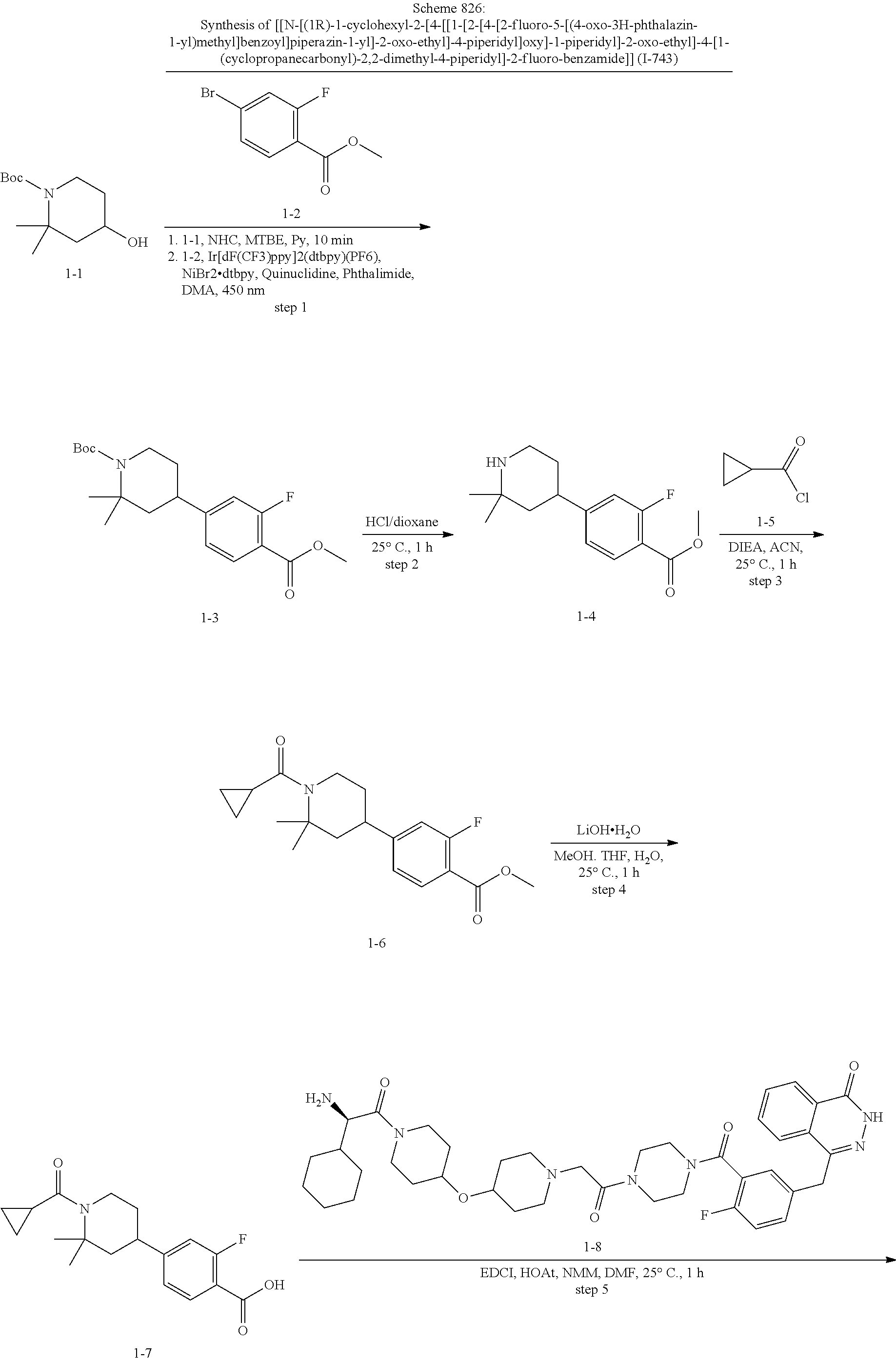

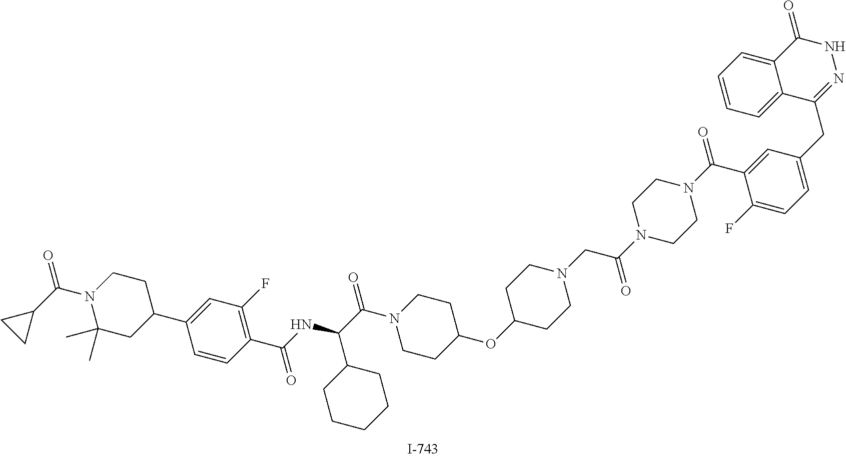 [SMILES: CC1(C)CC(c2ccc(C(=O)N[C@@H](C(=O)N3CCC(OC4CCN(CC(=O)N5CCN(C(=O)c6cc(Cc7n[nH]c(=O)c8ccccc78)ccc6F)CC5)CC4)CC3)C3CCCCC3)c(F)c2)CCN1C(=O)C1CC1]

### Step 1: Synthesis of Intermediate 1-3

To a 40 mL vial equipped with a stir bar was added intermediate 1-1 (573.45 mg, 2.50 mmol, 1.75 eq) and NHC (903.66 mg, 2.29 mmol, 1.6 eq) in MTBE (9 mL). Pyridine (180.85 mg, 2.29 mmol, 184.54 L, 1.6 eq) in MTBE (1 mL) was added dropwise. The vial was sealed and placed in a glovebox and stirred at 25° C. for 10 min, then filtered to give the filtrate. Ir(ppy)₂(dtbpy)(PF₆) (19.59 mg, 21.43 μmol, 0.015 eq), NiBr₂-dtbpy (34.79 mg, 71.45 μmol, 0.05 eq), quinuclidine (278.04 mg, 2.50 mmol, 1.75 eq) and intermediate 1-2 (333 mg, 1.43 mmol, 1 eq) in DMA (15 mL) were then added to the filtrate. The reaction was stirred and irradiated with a 10 W 455 nm blue LED lamp (3 cm away), with water cooling to keep the reaction temperature at 25° C. for 14 hrs. The reaction mixture was diluted with water (10 mL) and extracted with DCM 30 mL (10 mL*3). The combined organic layers were dried over Na₂SO₄, filtered and concentrated under reduced pressure to give a residue. The residue was purified by flash silica gel chromatography (ISCO; 20 g SepaFlash Silica Flash Column, Eluent of 0˜100% Petroleum ether/Ethyl acetate @40 mL/min). The eluent was concentrated under reduced pressure to give product. Intermediate 1-3 (0.75 g, crude) was obtained as a colorless oil. LCMS (Method D): Retention time: 0.657 min, [M+H]⁺=365.2.

### Step 2: Synthesis of Intermediate 1-4

A solution of intermediate 1-3 (700 mg, 1.92 mmol, 1 eq) in HCl/dioxane (2 M, 7.00 mL, 7.31 eq) was stirred at 25° C. for 1 hr. The reaction mixture was concentrated under reduced pressure to give product. Intermediate 1-4 (700 mg, crude) was obtained as a yellow oil. LCMS (Method D): Retention time: 0.239 min, [M+H]⁺=266.0.

### Step 3: Synthesis of Intermediate 1-6

To a solution of intermediate 1-4 (500 mg, 1.66 mmol, 1 eq, HCl salt) and intermediate 1-5 (173.19 mg, 1.66 mmol, 150.34 μL, 1 eq) in ACN (5 mL) was added DIEA (428.26 mg, 3.31 mmol, 577.17 μL, 2 eq). The mixture was stirred at 25° C. for 1 hr. The reaction mixture was diluted with water (5 mL) and extracted with DCM (5 mL*3). The combined organic layers were dried over Na₂SO₄, filtered and concentrated under reduced pressure to give a residue. The residue was purified by flash silica gel chromatography (ISCO; 40 g SepaFlash Silica Flash Column, Eluent of 0-50% PE/EA@40 mL/min). The eluent was concentrated under reduced pressure to give product. Intermediate 1-6 (260 mg, 717.46 mol, 43.30% yield, 92% purity) was obtained as a yellow oil. LCMS (Method D): Retention time: 0.453 min, [M+H]⁺=334.1. ¹H NMR (400 MHz, METHANOL-d4) δ=7.88 (t, J=8.0 Hz, 1H), 7.21-7.20 (m, 1H), 7.18-7.15 (m, 1H), 4.13-4.16 (m, 1H), 3.89 (s, 3H), 3.61-3.54 (m, 1H), 3.15-3.01 (m, 1H), 2.17-2.07 (m, 1H), 1.95-1.86 (m, 2H), 1.83-1.72 (m, 1H), 1.68-1.62 (m, 1H), 1.55 (s, 3H), 1.48 (s, 3H), 0.95-0.86 (m, 2H), 0.77-0.69 (m, 2H). ¹⁹F NMR (377 MHz, METHANOL-d4) δ=−111.40 (s, 1F).

### Step 4: Synthesis of Intermediate 1-7

To a solution of intermediate 1-6 (200 mg, 599.89 μmol, 1 eq) in MeOH (0.7 mL), THF (0.7 mL) and H₂O (0.7 mL) was added LiOH H₂O (50.34 mg, 1.20 mmol, 2 eq). The mixture was stirred at 25° C. for 0.5 hr. The mixture was adjusted to pH=5 with citric acid and extracted with EA (5 mL*3). The combined organic layers were dried over Na₂SO₄, filtered and concentrated under reduced pressure to give product. Intermediate 1-7 (200 mg, crude) was obtained as a colorless oil. LCMS (Method D): Retention time: 0.382 min, [M+H]⁺=320.2. SCF: Retention time: 1.269 min. 1.400 min.

### Step 5: Synthesis of I-743

To a solution of intermediate 1-7 (40 mg, 125.25 μmol, 1 eq) in DMF (1 mL) was added HOAt (17.05 mg, 125.25 μmol, 17.52 μL, 1 eq), EDCI (72.03 mg, 375.74 μmol, 3 eq), NMM (63.34 mg, 626.23 μmol, 68.85 μL, 5 eq), and intermediate 1-8 (95.98 mg, 125.25 μmol, 1 eq, HCl salt). The mixture was stirred at 25° C. for 1 hr. The reaction mixture was diluted with water (5 mL) and extracted with DCM (5 mL*3). The combined organic layers were dried over Na₂SO₄, filtered and concentrated under reduced pressure to give a residue. The residue was purified by reversed-phase chromatography (0.1% FA condition). The eluent was lyophilized to give a product. I-743 (40 mg, 37.13 μmol, 29.65% yield, 100% purity, FA salt) was obtained as a white solid. LCMS (Method D): Retention time: 0.410 min, [M+H]⁺=1031.6. SCF: Retention time: 1.320 min. 1.471 min. ¹H NMR (400 MHz, METHANOL-d4) δ=8.50 (s, 1H), 8.37 (d, J=7.6 Hz, 1H), 7.99-7.91 (m, 1H), 7.90-7.80 (m, 2H), 7.73 (t, J=8.0 Hz, 1H), 7.49 (s, 1H), 7.37 (s, 1H), 7.24-7.13 (m, 3H), 4.99 (d, J=7.6 Hz, 1H), 4.38 (s, 2H), 4.13-3.96 (m, 2H), 3.95-3.83 (m, 1H), 3.77 (d, J=17.6 Hz, 3H), 3.68 (s, 2H), 3.64-3.41 (m, 7H), 3.40-3.33 (m, 2H), 3.14-3.03 (m, 1H), 2.89 (s, 2H), 2.56-2.34 (m, 2H), 2.17-2.06 (m, 1H), 1.99-1.58 (m, 18H), 1.55 (s, 4H), 1.48 (s, 3H), 1.35-1.06 (m, 5H), 0.96-0.87 (m, 1H), 0.83-0.67 (m, 3H). ¹⁹F NMR (376 MHz, METHANOL-d4)

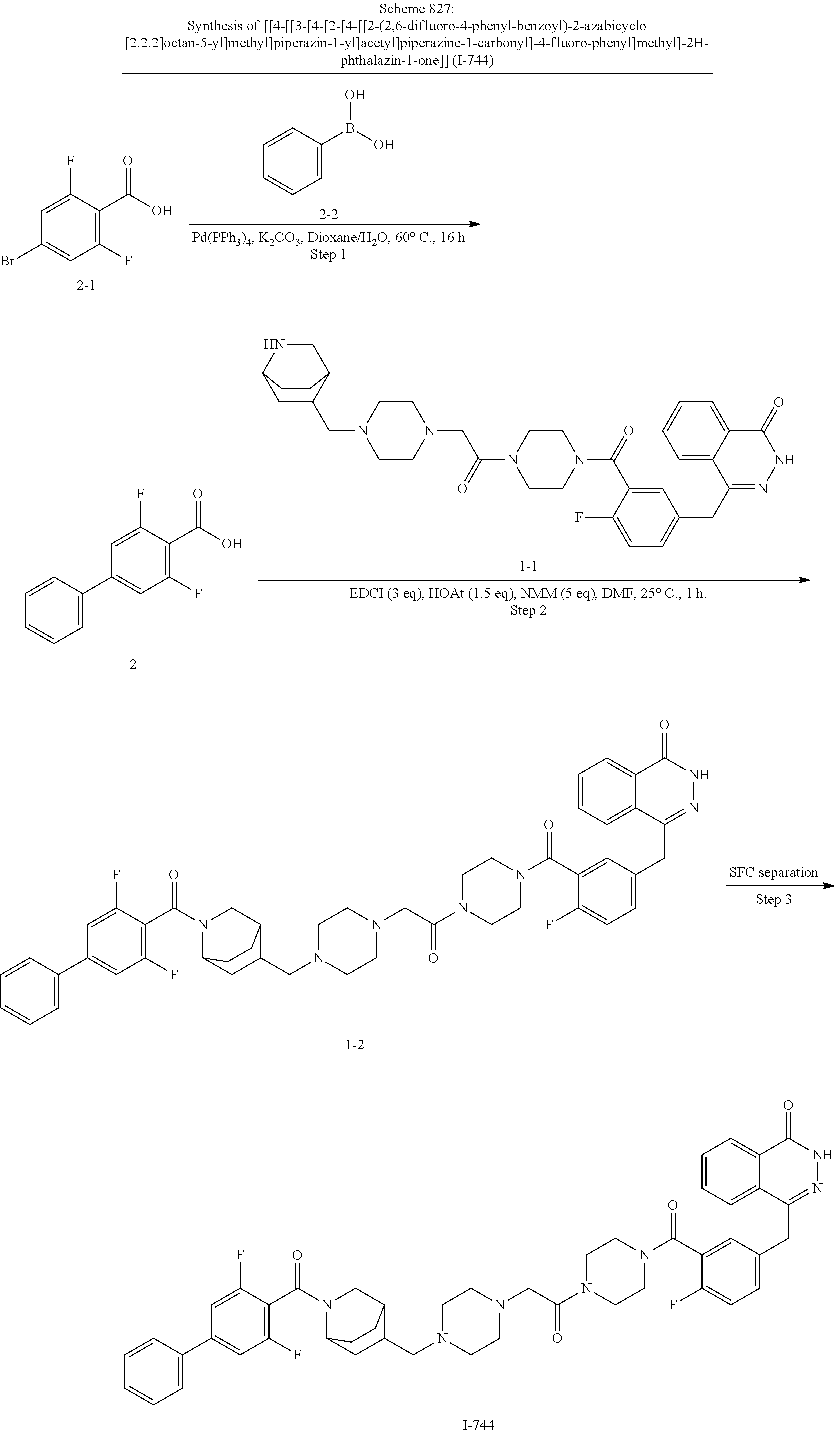

### Step 1: Synthesis of Intermediate 2

To a solution of Intermediate 2-1 (10 g, 42.19 mmol, 1 eq) in dioxane (100 mL) was added H₂O (10 mL) and Pd(PPh₃)₄ (2.44 g, 2.11 mmol, 0.05 eq), K₂CO₃ (11.66 g, 84.39 mmol, 2 eq) and Intermediate 2-2 (6.17 g, 50.63 mmol, 1.2 eq). The system was purged with nitrogen three times. Then the system was allowed to reach 60° C. and stirred for 16 hrs under inert atmosphere. The reaction mixture was concentrated under reduced pressure to remove dioxane (100 mL). Then the residue was diluted with water (150 mL) and acidified with 2N HCl to pH=2. The suspension was filtered, and the solid was filtered off and collected as crude product. After work-up, Intermediate 2 (8 g, crude) was obtained as a black solid. LCMS (Method E): Rt=0.512 min, [M+H]⁺=235.2.

### Step 2: Synthesis of Intermediate 1-2

To a solution of Intermediate 1 (150 mg, 640.48 μmol, 1 eq) in DMF (4 mL) was added EDCI (368.34 mg, 1.92 mmol, 3 eq), HOAt (130.77 mg, 960.73 μmol, 134.39 μL, 1.5 eq), NMM (323.92 mg, 3.20 mmol, 352.08 μL, 5 eq), Intermediate 1-1 (417.72 mg, 640.48 μmol, 1 eq, HCl salt). The resulting suspension was stirred at 20° C. for another 1 h. The mixture was quenched with water (20 mL) and extracted with EA (10 mL*5), washed with brine (10 mL*2), the combined organic layer was dried over Na₂SO₄, filtered and concentrated under reduced pressure to give a residue. The crude product was purified by reverse phase chromatography (40 g of XB—C18, 20-40 μm, 120 Å) Mobile phase: A for H₂O (0.1% FAv/v) and B for acetonitrile; Gradient: B5%-100% in 30 min; Flow rate: 100 ml/min; Column temperature: R.T. Wavelength: 220 nm/254 nm). After purification, the collected fractions were concentrated to remove organic solvents and lyophilized to give product. The product was re-purified by prep-HPLC (column: Waters Xbridge Prep OBD C18 150*40 mm*10 um; mobile phase: [water(NH₄HCO₃)-ACN]; gradient: 32%-62% B over 15 min). After purification, the eluent was concentrated to remove organic solvents and lyophilized to give product. Intermediate 1-2 (300 mg, 358.19 μmol, 55.93% yield, 99.330% purity) was obtained as a white solid. LCMS (Method G): Rt=1.850 min, [M+H]⁺=832.5. SCF: Rt=1.693 min, 1.889 min, 2.140 min, 2.603 min. ¹H NMR (400 MHz, DMSO-d6) δ=12.59 (s, 1H), 8.26 (d, J=7.6 Hz, 1H), 8.01-7.93 (m, 1H), 7.92-7.80 (m, 2H), 7.77 (d, J=7.4 Hz, 2H), 7.61-7.54 (m, 2H), 7.53-7.42 (m, 4H), 7.37 (br s, 1H), 7.23 (t, J=8.8 Hz, 1H), 4.33 (s, 2H), 3.71-3.34 (m, 9H), 3.22-3.08 (m, 4H), 2.46-2.16 (m, 9H), 1.97 (br s, 2H), 1.92-1.58 (m, 5H), 1.55-1.09 (m, 2H).

### Step 3: Synthesis of I-744

Intermediate 1-2 (300 mg) was purified by SFC (Sample preparation: EtOH (15 mL) was added to the sample; Column: Daicel Chiralpak IK 250×30 mm I.D., 10 μm particle size; Instrument: Waters 150 Preparative SFC system: 65% EtOH+ACN (BASE) in Supercritical CO₂ Flow Rate: 100 g/min; Cycle Time: 3 min, Single injection volume: 4.0 mL; Back Pressure: 80 bar to keep the CO₂ in Supercritical flow). After purification, the eluent was concentrated to remove organic solvents and lyophilized to give I-744 (205 mg, 246.42 μmol, 68.33% yield) as a white solid. [3247](Mixture 2) (50 mg, 60.10 μmol, 16.67% yield, N/A purity) was obtained as a white solid which was checked by LCMS, SFC

LCMS: Rt=0.480 min, [M+H]⁺=832.5 HPLC: Rt=10.435 min, 10.654 min

SFC: Rt=1.676 min, 1.868 min, 2.116 min, 2.567 min. SFC: Rt=1.843 min, 2.571 min. ¹H NMR (400 MHz, DMSO-d6) δ=12.59 (s, 1H), 8.26 (d, J=7.6 Hz, 1H), 8.00-7.94 (m, 1H), 7.93-7.80 (m, 2H), 7.77 (d, J=7.6 Hz, 2H), 7.56 (d, J=9.2 Hz, 2H), 7.52-7.42 (m, 4H), 7.37 (br s, 1H), 7.24 (t, J=8.8 Hz, 1H), 4.33 (s, 2H), 3.74-3.34 (m, 9H), 3.23-3.04 (m, 4H), 2.47-2.09 (m, 9H), 2.07-1.94 (m, 2H), 1.79 (br s, 5H), 1.55-1.11 (m, 2H). ¹⁹F NMR (377 MHz, DMSO-d6) δ=−113.23-−115.19 (m, 2F), −119.77 (br s, 1F)

(Mixture 2) (50 mg, 60.10 μmol, 16.67% yield, N/A purity) was re-purified by SFC SFC(Sample preparation: Add EtOH 15 mL into sample; Column: Daicel Chiralpak IK 250×30 mm I.D., 10 μm particle size; Instrument: Waters 150 Preparative SFC system: 65% EtOH+ACN (BASE) in Supercritical C02 Flow Rate: 100 g/min; Cycle Time: 3 min, Single injection volume: 4.0 mL; Back Pressure: 80 bar to keep the C02 in Supercritical flow), after purification, then concentrated to remove organic solvents to give:

Mixture 2_1 (15 mg, 18.03 μmol, 30.00% yield, N/A purity) was obtained as a white solid. Mixture 2_2 was obtained as a white solid.

LCMS: Rt=0.474 min, [M+H]⁺=832.4. SCF: Rt=1.824 min, 2.541 min. SCF: Rt=1.834 min, 2.550 min. ¹H NMR (400 MHz, DMSO-d6) δ=12.60 (s, 1H), 8.26 (d, J=7.6 Hz, 1H), 8.01-7.94 (m, 1H), 7.92-7.81 (m, 2H), 7.80-7.75 (m, 2H), 7.62-7.55 (m, 2H), 7.53-7.43 (m, 4H), 7.35 (br s, 3H), 7.28-7.19 (m, 4H), 7.10 (br s, 2H), 4.33 (s, 2H), 3.70-3.39 (m, 9H), 3.27-3.15 (m, 4H), 2.26-1.86 (m, 4H), 1.72-1.49 (m, 3H), 1.47-1.20 (m, 3H)

VT ¹H NMR (400 MHz, DMSO-d6) δ=12.37 (s, 1H), 8.28 (d, J=7.6 Hz, 1H), 7.98-7.93 (m, 1H), 7.91-7.79 (m, 2H), 7.75 (d, J=7.2 Hz, 2H), 7.54-7.41 (m, 6H), 7.34 (d, J=5.6 Hz, 1H), 7.29-7.07 (m, 6H), 4.54-4.29 (m, 2H), 3.74-3.39 (m, 9H), 2.97-2.70 (m, 7H), 2.31-1.93 (m, 4H), 1.76-1.49 (m, 3H), 1.44-1.23 (m, 3H)

¹H NMR (400 MHz, DMSO-d6) δ=12.59 (s, 1H), 8.26 (d, J=7.6 Hz, 1H), 8.02-7.94 (m, 1H), 7.93-7.74 (m, 4H), 7.61-7.54 (m, 2H), 7.53-7.41 (m, 4H), 7.37 (t, J=6.8 Hz, 1H), 7.24 (t, J=8.8 Hz, 1H), 4.34 (d, J=5.2 Hz, 2H), 3.67-3.47 (m, 6H), 3.42-3.34 (m, 3H), 3.21-3.06 (m, 4H), 2.46-2.19 (m, 9H), 2.04-1.78 (m, 3H), 1.75-1.43 (m, 4H), 1.29-1.10 (m, 2H)

VT ¹NMR (400 MHz, DMSO-d6) δ=12.39 (s, 1H), 8.28 (d, J=7.6 Hz, 1H), 7.98-7.92 (m, 1H), 7.91-7.79 (m, 2H), 7.75 (d, J=7.2 Hz, 2H), 7.52-7.41 (m, 6H), 7.35 (d, J=6.4 Hz, 1H), 7.20 (t, J=8.8 Hz, 1H), 4.33 (s, 2H), 4.14-4.04 (m, 1H), 3.72-3.52 (m, 5H), 3.43 (d, J=7.6 Hz, 2H), 3.31-3.12 (m, 4H), 2.46-2.22 (m, 10H), 2.04-1.81 (m, 3H), 1.78-1.50 (m, 4H), 1.42-1.13 (m, 2H)

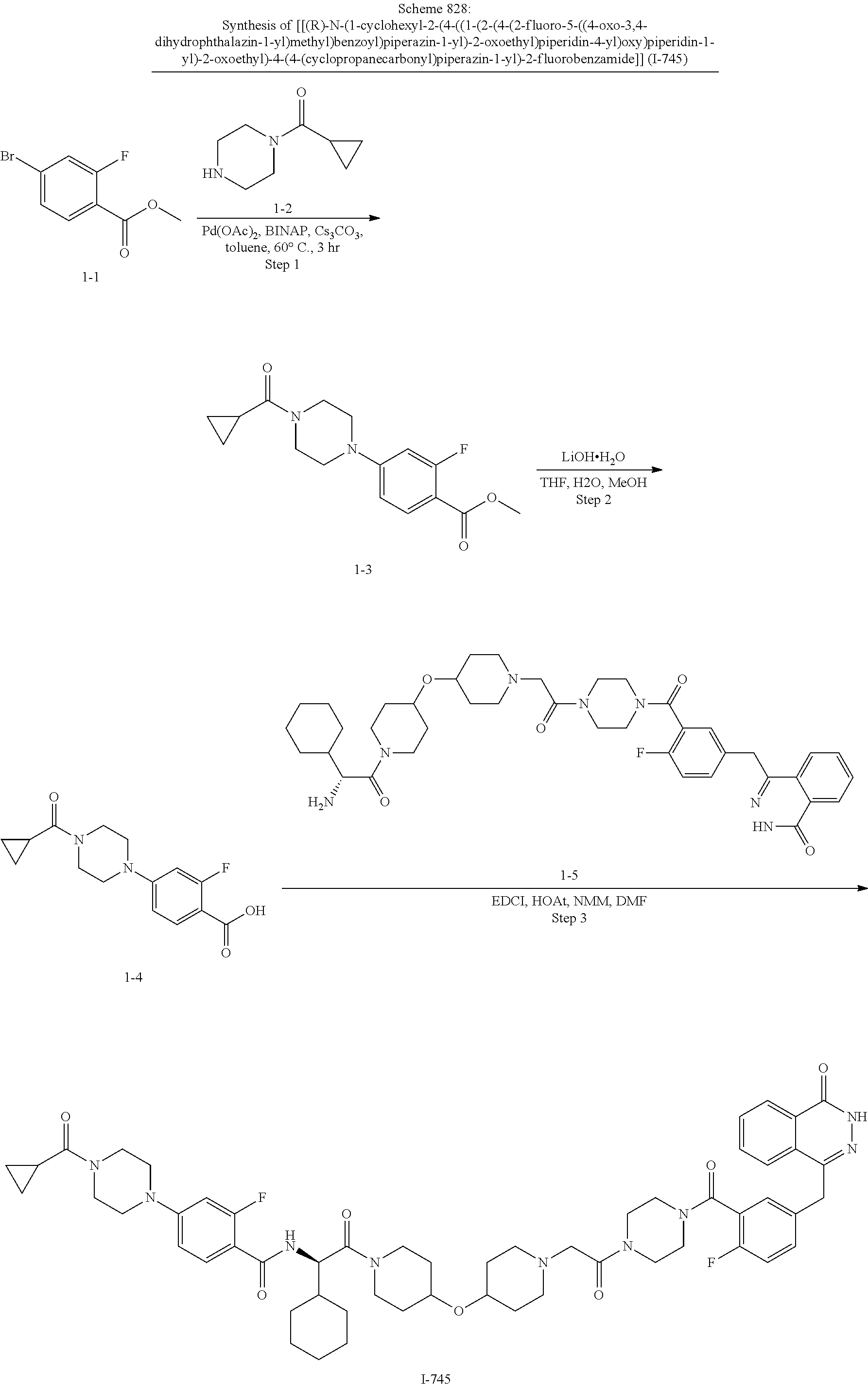

### Step 1: Synthesis of Intermediate 1-3

To a solution of intermediate 1-1 (100 mg, 429.12 μmol, 1 eq) and intermediate 1-2 (66.17 mg, 429.12 μmol, 1 eq) in toluene (1 mL) was added Pd(OAc)₂ (9.63 mg, 42.91 μmol, 0.1 eq) and BINAP (26.72 mg, 42.91 μmol, 0.1 eq) and Cs₂CO₃ (419.45 mg, 1.29 mmol, 3 eq). The mixture was stirred at 60° C. for 1 hr. The reaction mixture was quenched with H₂O (2 mL) and then extracted with DCM (2 mL*3), the combined organic phase was dried over Na₂SO₄, filtered and concentrated to give crude product. The crude was purified by flash silica gel chromatography (ISCO; 12 g SepaFlash Silica Flash Column, Eluent of 0˜100% EA/PE @30 mL/min) and the eluent was concentrated to give product. Intermediate 1-3 (60 mg, 195.87 μmol, 45.64% yield) was obtained as a white solid. LCMS (Method D): Retention time: 0.333 min, [M+H]⁺=307.1.

### Step 2: Synthesis of Intermediate 1-4

To a solution of intermediate 1-3 (50 mg, 163.22 μmol, 1 eq) in THF (0.2 mL), H₂O (0.2 mL) and MeOH (0.2 mL) was added LiOH H₂O (13.70 mg, 326.44 μmol, 2 eq). The mixture was stirred at 25° C. for 1 hr. The mixture was adjusted to pH=7 with aq. HCl (1 M) and then extracted with DCM (10 mL*3). The combined organic phase was dried over Na₂SO₄, filtered and concentrated to give product. Intermediate 1-4 (40 mg, crude) was obtained as a white solid. LCMS (Method D): Retention time: 0.289 min, [M+H]⁺=293.1.

### Step 3: Synthesis of I-745

To a solution of Intermediate 1-4 (30 mg, 102.63 μmol, 1 eq) and intermediate 1-5 (74.91 mg, 97.75 μmol, 9.52e−1 eq, HCl salt) in DMF (0.3 mL) was added EDCI (39.35 mg, 205.27 μmol, 2 eq), HOAt (13.97 mg, 102.63 μmol, 14.36 μL, 1 eq) and NMM (51.90 mg, 513.16 μmol, 56.42 μL, 5 eq). The mixture was stirred at 25° C. for 1 hr. The reaction mixture was added into H₂O (1 mL) and extracted with DCM (1 mL*3), the combined organic phase was dried over Na₂SO₄, filtered and concentrated to give crude product. The residue was purified by prep-HPLC (column: CD01-Phenomenex luna C18 150*25*10 um; mobile phase: [Water (FA)-MeCN]; gradient: 21%-51% B over 8 min). The eluent was concentrated to remove ACN and lyophilized to give product. I-745 (18.61 mg, 17.57 μmol, 17.12% yield, 99.156% purity, FA salt) as a white solid. LCMS (Method D): Retention time: 0.389 min, [M+H]⁺=1004.5. SCF: Retention time: 2.203 min. ¹H NMR (400 MHz, METHANOL-d4) δ=8.55 (s, 1H), 8.37 (d, J=8.0 Hz, 1H), 7.99-7.92 (m, 1H), 7.91-7.80 (m, 2H), 7.79-7.70 (m, 1H), 7.54-7.45 (m, 1H), 7.37 (d, J=4.0 Hz, 1H), 7.16 (t, J=8.8 Hz, 1H), 6.88-6.80 (m, 1H), 6.75 (s, 1H), 5.01 (d, J=6.8 Hz, 1H), 4.45-4.34 (m, 2H), 4.08-3.85 (m, 4H), 3.83 (s, 6H), 3.69-3.64 (m, 1H), 3.62-3.50 (m, 4H), 3.49-3.41 (m, 4H), 3.40-3.34 (m, 4H), 3.21 (d, J=3.2 Hz, 1H), 2.86-2.70 (m, 2H), 2.38-2.19 (m, 2H), 2.04-1.97 (m, 1H), 1.97-1.84 (m, 4H), 1.84-1.71 (m, 5H), 1.70-1.55 (m, 4H), 1.54-1.45 (m, 1H), 1.36-1.20 (m, 3H), 1.18-1.08 (m, 2H), 0.94-0.88 (m, 2H), 0.87-0.79 (m, 2H). ¹⁹F NMR (377 MHz, METHANOL-d4) δ=−113.04 (br s, 1F), −120.66-−120.93 (m, 1F).

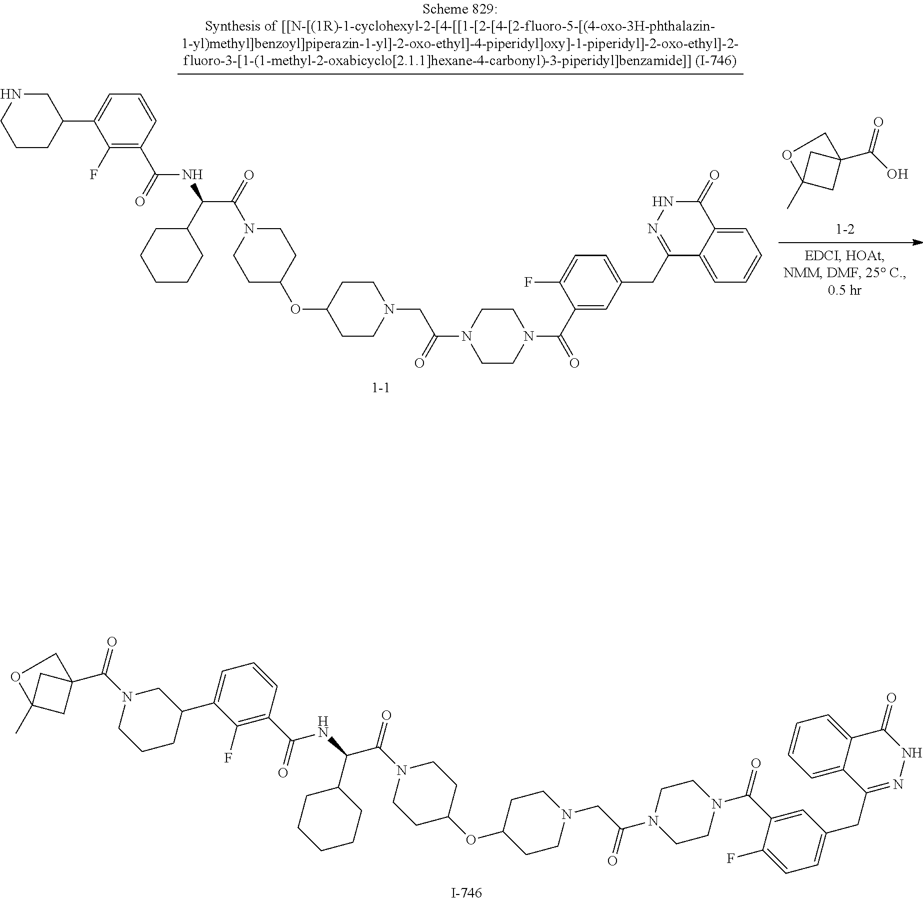

To a solution of intermediate 1-1 (450 mg, 463.17 μmol, 1 eq, HCl salt) and intermediate 1-2 (65.84 mg, 463.17 μmol, 1 eq) in DMF (5 mL) was added EDCI (266.37 mg, 1.39 mmol, 3 eq), HOAt (63.04 mg, 463.17 μmol, 64.79 μL, 1 eq) and NMM (234.25 mg, 2.32 mmol, 254.62 μL, 5 eq). The mixture was stirred at 25° C. for 0.5 hr. The reaction mixture was poured into H₂O (5 mL), and extracted with EA (5 mL*3). The combined organic layers were dried over Na₂SO₄, filtered and concentrated under reduced pressure to give a residue. The residue was purified by reversed phase chromatography (0.1% FA), the eluent was concentrated to remove MeCN and lyophilized to give product. I-746 (134.04 mg, 119.50 mol, 25.80% yield, 98.54% purity, FA salt) was obtained as a white solid. LCMS (Method D): Rt=0.410 min, [M+H]⁺=1059.6. SCF: Rt=1.930 min, 2.542 min, ¹H NMR (400 MHz, METHANOL-d4) δ=8.49-8.41 (m, 1H), 8.37 (d, J=8.0 Hz, 1H), 8.00-7.92 (m, 1H), 7.92-7.83 (m, 2H), 7.82-7.77 (m, 3H), 7.76-7.73 (m, 1H), 7.50 (s, 1H), 7.42-7.32 (m, 1H), 7.30 (d, J=7.6 Hz, 1H), 7.17 (t, J=8.8 Hz, 1H), 4.71 (d, J=12.0 Hz, 1H), 4.39 (s, 2H), 3.82-3.72 (m, 2H), 3.71-3.60 (m, 3H), 3.55-3.48 (m, 2H), 3.42 (s, 1H), 3.34 (d, J=2.0 Hz, 2H), 3.26-3.17 (m, 1H), 3.03-2.79 (m, 4H), 2.78-2.55 (m, 8H), 2.49-2.37 (m, 2H), 2.02-1.89 (m, 2H), 1.83 (d, J=13.2 Hz, 1H), 1.36 (t, J=7.6 Hz, 3H), 1.29-1.16 (m, 2H). ¹⁹F NMR (377 MHz, METHANOL-d4) δ=−120.702, −120.762, −121.603, −121.633, −121.693, −121.715, −121.790, −121.813.

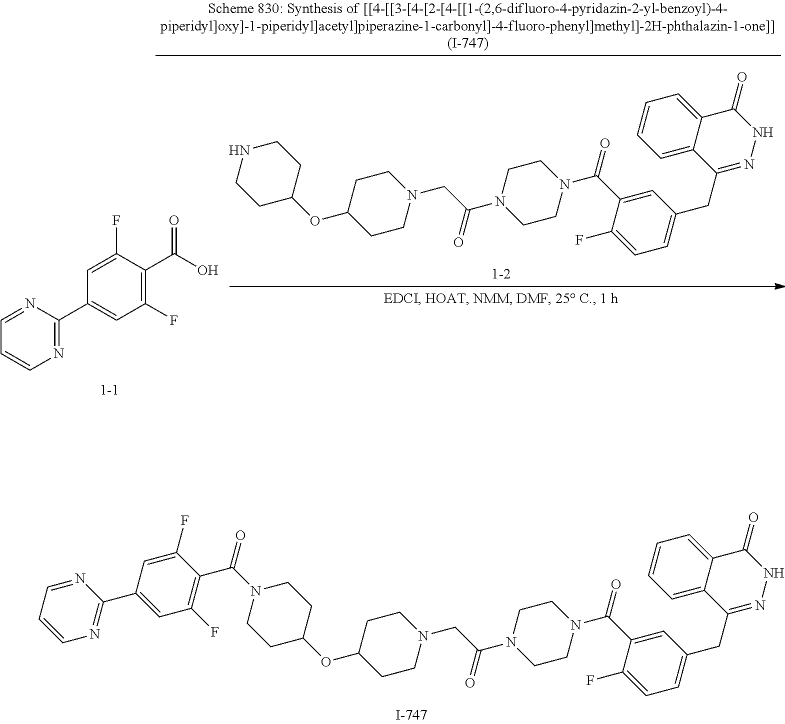 [SMILES: O=C(O)c1c(F)cc(-c2ncccn2)cc1F ; O=C(CN1CCC(OC2CCNCC2)CC1)N1CCN(C(=O)c2cc(Cc3n[nH]c(=O)c4ccccc34)ccc2F)CC1 ; O=C(CN1CCC(OC2CCN(C(=O)c3c(F)cc(-c4ncccn4)cc3F)CC2)CC1)N1CCN(C(=O)c2cc(Cc3n[nH]c(=O)c4ccccc34)ccc2F)CC1]

To a solution of intermediate 1-1 (109.95 mg, 465.56 μmol, 1.1 eq) in DMF (2.5 mL) was added EDCI (243.40 mg, 1.27 mmol, 3 eq), HOAT (57.61 mg, 423.24 μmol, 59.21 μL, 1 eq) and NMM (214.05 mg, 2.12 mmol, 232.66 μL, 5 eq). The mixture was stirred at 25° C. for 15 mins. Then intermediate 1-2 (250 mg, 423.24 μmol, 1 eq) was added into the mixture and then it was stirred at 25° C. for 1 hr. The mixture was poured into H₂O (2 mL) and extracted with EA (3 mL*3). The organic layers were dried over Na₂SO₄, filtered and concentrated under reduced pressure to give a residue. The crude product was purified by reversed phase chromatography (0.1% FA) and lyophilized to give product. I-747 (92.12 mg, 107.51 μmol, 25.40% yield, 99.767% purity, FA salt) was obtained as a white solid. LCMS (Method D): Retention time: 0.328 min, [M+H]⁺=809.3. ¹H NMR (400 MHz, METHANOL-d4) δ=8.89 (d, J=4.8 Hz, 2H), 8.47 (s, 1H), 8.39-8.33 (m, 1H), 8.13 (d, J=9.2 Hz, 2H), 7.99-7.92 (m, 1H), 7.90-7.79 (m, 2H), 7.49 (d, J=2.4 Hz, 1H), 7.44 (t, J=4.8 Hz, 1H), 7.37 (t, J=5.6 Hz, 1H), 7.16 (t, J=9.2 Hz, 1H), 4.38 (s, 2H), 4.12-4.01 (m, 1H), 3.81 (d, J=4.0 Hz, 2H), 3.75 (d, J=4.4 Hz, 1H), 3.72-3.45 (m, 10H), 3.42 (s, 1H), 3.30-3.22 (m, 1H), 3.08-2.93 (m, 2H), 2.75-2.55 (m, 2H), 1.96 (s, 3H), 1.90-1.82 (m, 1H), 1.81-1.64 (m, 3H), 1.64-1.54 (m, 1H). ¹⁹F NMR (377 MHz, METHANOL-d4) δ=−120.749, −115.143.

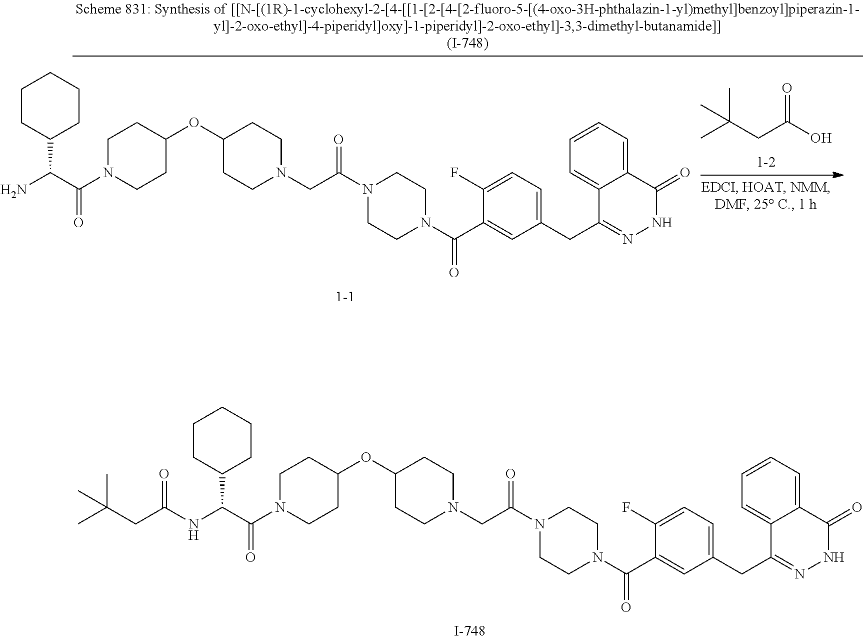

To a solution of intermediate 1-2 (60 mg, 516.54 μmol, 65.79 μL, 1 eq) in DMF (4 mL) was added HOAt (70.31 mg, 516.54 μmol, 72.26 μL, 1 eq), EDCI (297.06 mg, 1.55 mmol, 3 eq), NMM (261.23 mg, 2.58 mmol, 283.95 μL, 5 eq), and intermediate 1-1 (395.85 mg, 516.54 μmol, 1 eq, HCl salt). The mixture was stirred at 25° C. for 1 hr. The reaction mixture was diluted with water (10 mL) and extracted with DCM (5 mL*3). The combined organic layers were dried over Na₂SO₄, filtered and concentrated under reduced pressure to give a residue. The residue was purified by reversed-phase chromatography (0.1% FA condition). The eluent was lyophilized to give product. I-748 (273 mg, 311.31 μmol, 60.27% yield, 99.670% purity, FA salt) was obtained as a white solid. LCMS (Method D): Retention time: 0.366 min, [M+H]⁺=828.6 SCF: Retention time: 1.296 min. ¹H NMR (400 MHz, METHANOL-d4) δ=8.48 (s, 1H), 8.37 (d, J=7.6 Hz, 1H), 7.98-7.78 (m, 4H), 7.50 (d, J=2.4 Hz, 1H), 7.38 (t, J=5.2 Hz, 1H), 7.17 (t, J=9.2 Hz, 1H), 4.70 (d, J=8.0 Hz, 1H), 4.39 (s, 2H), 4.00 (d, J=6.0 Hz, 1H), 3.93-3.73 (m, 4H), 3.72-3.48 (m, 7H), 3.45 (s, 2H), 3.37-3.33 (m, 1H), 3.27-3.11 (m, 1H), 3.01-2.86 (m, 2H), 2.67-2.40 (m, 2H), 2.12 (s, 2H), 1.93 (s, 3H), 1.84-1.43 (m, 11H), 1.31-1.15 (m, 3H), 1.09-0.98 (m, 11H). ¹⁹F NMR (376 MHz, METHANOL-d4) δ=−120.75 (br d, J=19.8 Hz, 1F).

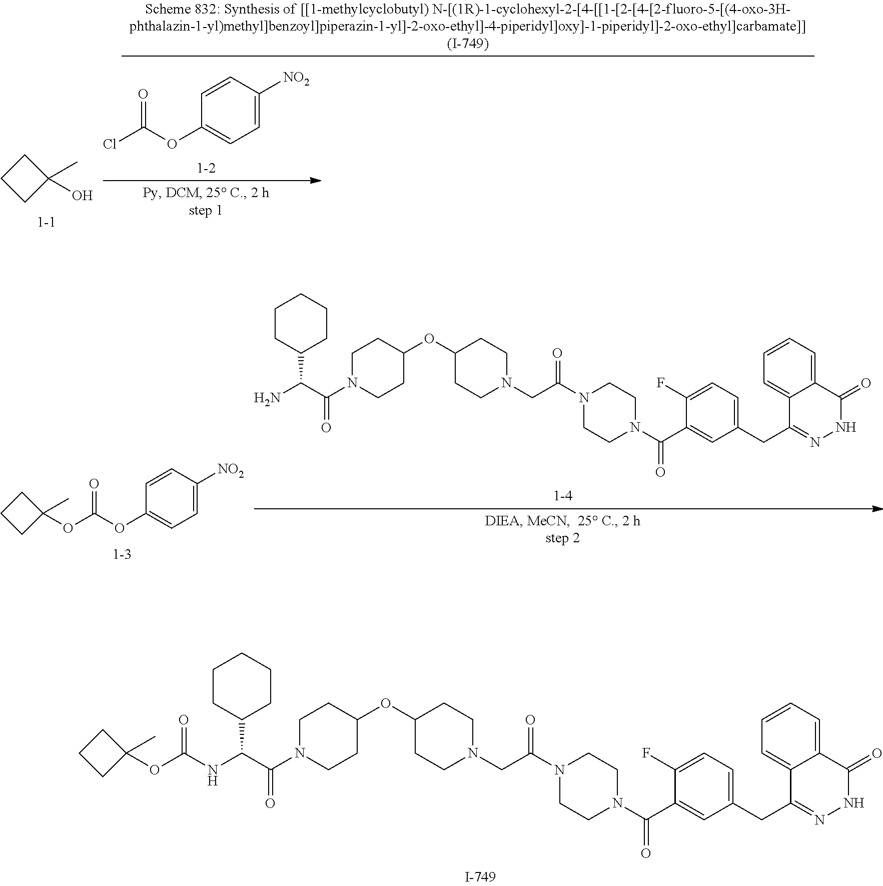

### Step 1: Synthesis of Intermediate 1-3

To a solution of intermediate 1-1 (100 mg, 1.16 mmol, 1 eq) and pyridine (110.20 mg, 1.39 mmol, 112.45 μL, 1.2 eq) in DCM (1 mL) was added intermediate 1-2 (280.82 mg, 1.39 mmol, 1.2 eq) at 0° C. The mixture was stirred at 25° C. for 3 hrs. The residue was diluted with H₂O (5 mL) and extracted with DCM (5 mL*3), dried over Na₂SO₄, filtered and concentrated under reduced pressure to give product. Intermediate 1-3 (290 mg, 1.15 mmol, 99.42% yield) was obtained as a yellow oil. LCMS (method D): Rt=0.438 min, [M+Na]⁺=274.8.

### Step 2: Synthesis of I-749

To a solution of intermediate 1-3 (120 mg, 477.64 μmol, 1 eq) and intermediate 1-4 (366.04 mg, 477.64 μmol, 1 eq, HCl) in ACN (3 mL) was added DIEA (123.46 mg, 955.28 μmol, 166.39 μL, 2 eq). The mixture was stirred at 25° C. for 1 hr. The reaction mixture was concentrated under reduced pressure to give a residue. The crude product was purified by reversed-phase chromatography (0.1% NH₄HCO₃ condition). The eluent was concentrated under reduced pressure to remove ACN, then lyophilized to afford product. I-749 (152 mg, 177.36 μmol, 37.13% yield, 98.25% purity) was obtained as a white solid. LCMS (Method D): Rt=0.401 min, [M+H]⁺=842.5. SCF: Rt=0.796 min. ¹H NMR (400 MHz, METHANOL-d4) δ=8.37 (d, J=6.8 Hz, 1H), 7.99-7.92 (m, 1H), 7.91-7.80 (m, 2H), 7.49 (br s, 1H), 7.38 (br s, 1H), 7.16 (t, J=8.8 Hz, 1H), 4.42-4.33 (m, 3H), 4.08-3.88 (m, 1H), 3.84-3.64 (m, 6H), 3.61-3.45 (m, 4H), 3.41-3.35 (m, 1H), 3.27-3.15 (m, 3H), 2.86-2.68 (m, 2H), 2.35-2.20 (m, 4H), 2.13-2.02 (m, 2H), 1.93-1.72 (m, 8H), 1.70-1.54 (m, 7H), 1.51 (s, 4H), 1.44-0.92 (m, 6H). ¹⁹F NMR (377 MHz, METHANOL-d4) δ=−120.80.

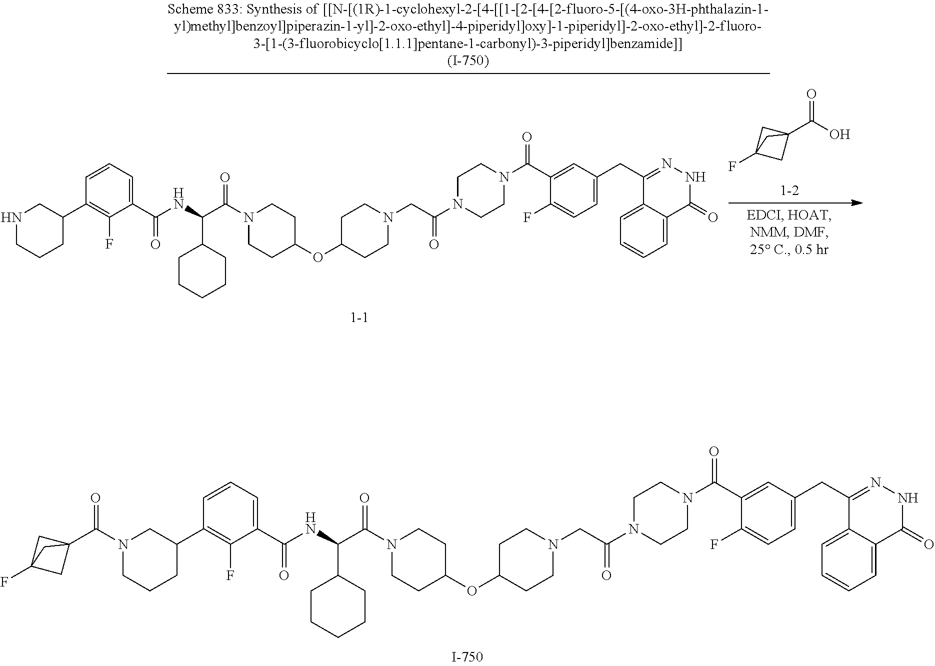 [SMILES: O=C(N[C@@H](C(=O)N1CCC(OC2CCN(CC(=O)N3CCN(C(=O)c4cc(Cc5n[nH]c(=O)c6ccccc56)ccc4F)CC3)CC2)CC1)C1CCCCC1)c1cccc(C2CCCNC2)c1F ; O=C(O)C12CC(F)(C1)C2 ; O=C(N[C@@H](C(=O)N1CCC(OC2CCN(CC(=O)N3CCN(C(=O)c4cc(Cc5n[nH]c(=O)c6ccccc56)ccc4F)CC3)CC2)CC1)C1CCCCC1)c1cccc(C2CCCN(C(=O)C34CC(F)(C3)C4)C2)c1F]

To a solution of intermediate 1-1 (400 mg, 411.70 μmol, 1 eq, HCl salt) and intermediate 1-2 (53.57 mg, 411.70 μmol, 1 eq) in DMF (4 mL) was added EDCI (236.77 mg, 1.24 mmol, 3 eq), HOAt (56.04 mg, 411.70 μmol, 57.59 μL, 1 eq) and NMM (208.21 mg, 2.06 mmol, 226.32 μL, 5 eq). The mixture was stirred at 25° C. for 0.5 hr. The reaction mixture was poured into H₂O (5 mL) and extracted with EA (5 mL*3). The combined organic layers were dried over Na₂SO₄, filtered and concentrated under reduced pressure to give a residue. The residue was purified by reversed phase chromatography (0.1% FA) then concentrated to remove MeCN and then lyophilized to give I-750 (170.13 mg, 161.94 μmol, 39.33% yield, 99.68% purity), as a white solid. LCMS (Method D): Rt=0.424 min, [M+H]⁺=1047.8. SCF: Rt=1.615 min, 2.121 min. ¹H NMR (400 MHz, METHANOL-d4) δ=8.38 (d, J=8.0 Hz, 1H), 8.00-7.92 (m, 1H), 7.92-7.81 (m, 2H), 7.60-7.46 (m, 3H), 7.38 (d, J=6.0 Hz, 1H), 7.32-7.22 (m, 1H), 7.17 (t, J=8.8 Hz, 1H), 5.02-4.94 (m, 1H), 4.63-4.53 (m, 2H), 4.39 (s, 2H), 4.14 (d, J=12.4 Hz, 1H), 4.09-3.99 (m, 1H), 3.97-3.84 (m, 1H), 3.83-3.72 (m, 4H), 3.68 (d, J=3.2 Hz, 1H), 3.61-3.45 (m, 5H), 3.27-3.25 (m, 1H), 3.24-3.17 (m, 2H), 3.17-3.11 (m, 1H), 2.86-2.70 (m, 3H), 2.49-2.39 (m, 6H), 2.33-2.21 (m, 2H), 2.08-2.00 (m, 1H), 1.96 (d, J=2.8 Hz, 11H), 1.74-1.47 (m, 8H), 1.40-1.04 (m, 6H). ¹⁹F NMR (377 MHz, METHANOL-d4) δ=−120.794, −121.395, −121.530, −121.560, −121.672, −121.710, −150.847, −150.959, −151.027.

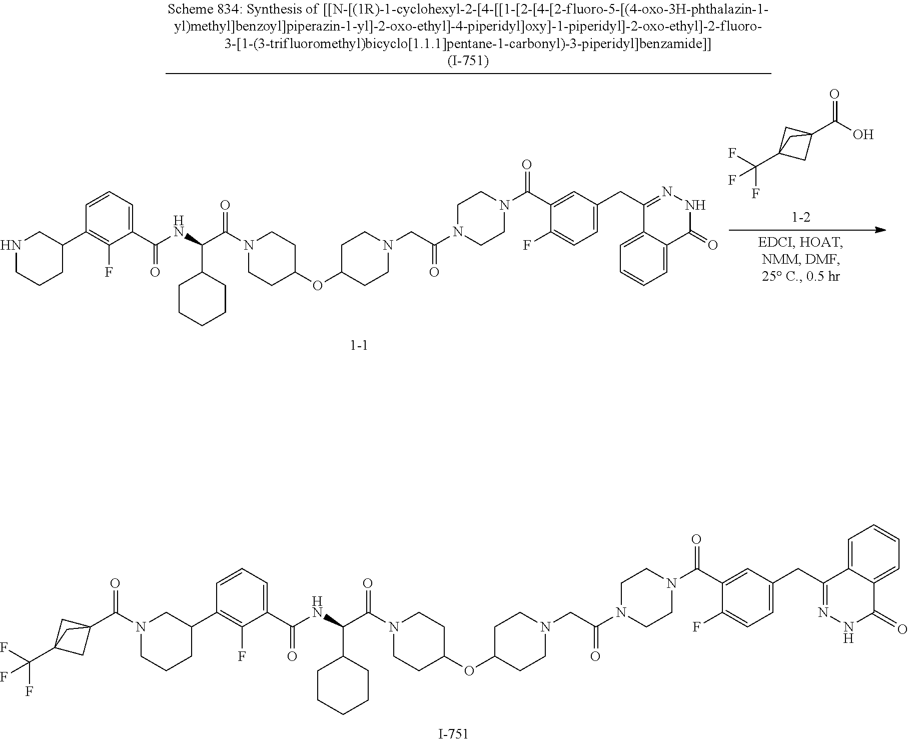 [SMILES: O=C(N[C@@H](C(=O)N1CCC(OC2CCN(CC(=O)N3CCN(C(=O)c4cc(Cc5n[nH]c(=O)c6ccccc56)ccc4F)CC3)CC2)CC1)C1CCCCC1)c1cccc(C2CCCNC2)c1F ; O=C(O)C12CC(C(F)(F)F)(C1)C2 ; O=C(N[C@@H](C(=O)N1CCC(OC2CCN(CC(=O)N3CCN(C(=O)c4cc(Cc5n[nH]c(=O)c6ccccc56)ccc4F)CC3)CC2)CC1)C1CCCCC1)c1cccc(C2CCCN(C(=O)C34CC(C(F)(F)F)(C3)C4)C2)c1F]

To a solution of intermediate 1-1 (400 mg, 411.70 μmol, 1 eq, HCl salt) and intermediate 1-2 (74.16 mg, 411.70 μmol, 1 eq) in DMF (4 mL) was added EDCI (236.77 mg, 1.24 mmol, 3 eq), HOAt (56.04 mg, 411.70 μmol, 57.59 μL, 1 eq) and NMM (208.21 mg, 2.06 mmol, 226.32 μL, 5 eq). The mixture was stirred at 25° C. for 0.5 hr. The reaction mixture was poured into H₂O (5 mL), and extracted with EA (5 mL*3). The combined organic layers were dried over Na₂SO₄, filtered and concentrated under reduced pressure to give a residue. The residue was purified by reversed phase chromatography (0.1% FA). The eluent was concentrated to remove MeCN and lyophilized to give I-751 (172.82 mg, 148.99 μmol, 36.19% yield, 98.56% purity, FA) as a white solid. LCMS (Method D): Rt=0.416 min, [M+H]⁺=1097.7. SCF: Rt=1.420 min, 1.818 min. ¹H NMR (400 MHz, METHANOL-d4) δ=8.59-8.45 (m, 1H), 8.38 (d, J=7.6 Hz, 1H), 7.96 (d, J=7.2 Hz, 1H), 7.92-7.81 (m, 2H), 7.62-7.47 (m, 3H), 7.39 (s, 1H), 7.32-7.22 (m, 1H), 7.17 (t, J=9.2 Hz, 1H), 4.97 (t, J=7.6 Hz, 1H), 4.60-4.53 (m, 1H), 4.40 (s, 2H), 4.27-4.18 (m, 1H), 4.09-3.99 (m, 1H), 3.98-3.85 (m, 1H), 3.81 (d, J=1.2 Hz, 2H), 3.76 (d, J=7.2 Hz, 1H), 3.72-3.66 (m, 2H), 3.64-3.47 (m, 5H), 3.46-3.40 (m, 1H), 3.27-3.13 (m, 2H), 3.12-2.98 (m, 1H), 2.92-2.69 (m, 3H), 2.48-2.30 (m, 8H), 2.06-2.00 (m, 1H), 2.00-1.74 (m, 11H), 1.74-1.60 (m, 6H), 1.56-1.47 (m, 1H), 1.39-1.22 (m, 3H), 1.22-1.03 (m, 3H). ¹⁹F NMR (377 MHz, METHANOL-d4) δ=−74.650, −74.680, −74.770, −74.800, −74.868, −120.749, −121.612, −121.725, −121.755, 121.830.

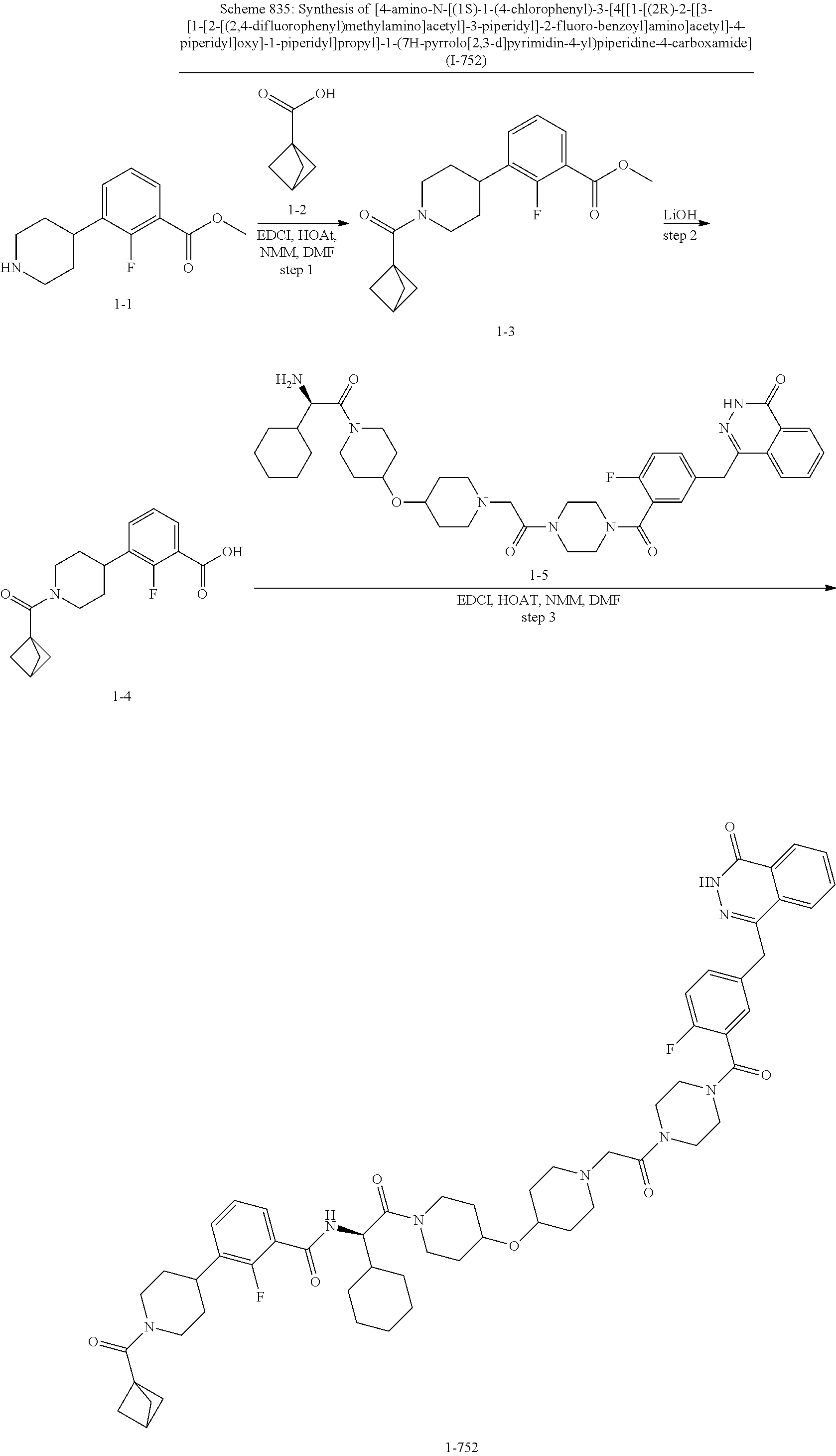

### Step 1: Synthesis of Intermediate 1-3

To a solution of intermediate 1-1 (1 g, 3.65 mmol, 1 eq, HCl salt) in DMF (10 mL) was added intermediate 1-2 (409.62 mg, 3.65 mmol, 1 eq), EDCI (2.10 g, 10.96 mmol, 3 eq), NMM (1.85 g, 18.27 mmol, 2.01 mL, 5 eq) and HOAt (497.24 mg, 3.65 mmol, 511.04 μL, 1 eq). The mixture was stirred at 25° C. for 1 hr. The reaction mixture was diluted with H₂O (10 mL) and extracted with EA (10 mL*4), the combined organic phase was dried over Na₂SO₄, filtered and concentrated under reduced pressure to give a residue. The residue was purified by flash silica gel chromatography (ISCO; 40 g SepaFlash Silica Flash Column, Eluent of 0˜1000% Petroleum ether: Ethyl acetate@35 mL/min). The eluent was concentrated under reduced pressure to give the product. Intermediate 1-3 (286 mg, 863.06 μmol, 23.62% yield, 100% purity) was obtained as a yellow solid. LCMS (Method D): Retention time: 0.436 min [M+H]⁺=332.0.

### Step 2: Synthesis of Intermediate 1-4

To a solution of intermediate 1-3 (200 mg, 603.54 μmol, 1 eq) in MeOH (1 mL), THF (1 mL) and H₂O (1 mL) was added LiOH·H₂O (75.98 mg, 1.81 mmol, 3 eq). The mixture was stirred at 25° C. for 0.5 hr. To the mixture was added 2 ml of H₂O and the pH was adjusted to 5 with 1M aq. HCl, and the mixture was extracted with EA (3 ml*3), and the combined organic phase was dried with anhydrous sodium sulfate, filtered and filtrate was concentrated to give the product. Intermediate 1-4 (180 mg, crude) was obtained as a white solid. LCMS (Method D): Retention time: 0.340 min, [M+H]3=318.1. ¹H NMR (400 MHz, METHANOL-d4) δ=7.80-7.74 (m, 1H), 7.54-7.47 (m, 1H), 7.21 (t, J=8.0 Hz, 1H), 4.65-4.60 (m, 1H), 4.46-4.41 (m, 1H), 3.28-3.19 (m, 2H), 2.77-2.75 (m, 1H), 2.21 (d, J=4.4 Hz, 5H), 1.97-1.80 (m, 2H), 1.77-1.56 (m, 3H), 1.24 (s, 2H). ¹⁹F NMR (377 MHz, METHANOL-d4) δ=−118.98 (s, 1F).

### Step 3: Synthesis of I-752

To a solution of Intermediate 1-4 (165.65 mg, 521.96 μmol, 1 eq) in DMF (4 mL) was added EDCI (300.18 mg, 1.57 mmol, 3 eq), NMM (263.97 mg, 2.61 mmol, 286.93 μL, 5 eq), intermediate 1-5 (400 mg, 521.96 μmol, 1 eq, HCl salt) and HOAt (71.04 mg, 521.96 μmol, 73.02 μL, 1 eq). The mixture was stirred at 25° C. for 0.5 hr. The reaction mixture was diluted with H₂O (10 mL) at 25° C. and extracted with EA (10 ml*3), the combined organic phase was dried with anhydrous sodium sulfate, filtered and the filtrate was concentrated to give crude product. The crude product was purified by reversed phase chromatography (0.1% FA condition) and the eluent was lyophilized to give the product. I-752 (240.26 mg, 219.92 μmol, 42.13% yield, 98.421% purity, FA salt) was obtained as a white solid. LCMS (Method D): Retention time: 0.405 min, [M+H]⁺=1029.5. SCF: Retention time: 3.410 min. ¹H NMR (400 MHz, METHANOL-d4) δ=8.50 (s, 1H), 8.37 (d, J=8.0 Hz, 1H), 7.95 (d, J=7.6 Hz, 1H), 7.91-7.80 (m, 2H), 7.58-7.42 (m, 3H), 7.42-7.33 (m, 1H), 7.26-7.13 (m, 2H), 4.97 (d, J=7.6 Hz, 1H), 4.67-4.58 (m, 1H), 4.47-4.36 (m, 3H), 4.09-3.97 (m, 1H), 3.96-3.83 (m, 1H), 3.77 (d, J=18.4 Hz, 3H), 3.68 (s, 2H), 3.64-3.50 (m, 4H), 3.47 (s, 2H), 3.38 (s, 2H), 3.29-3.17 (m, 3H), 2.90 (s, 2H), 2.75 (t, J=12.4 Hz, 1H), 2.58-2.38 (m, 3H), 2.26-2.16 (m, 6H), 2.01-1.74 (m, 11H), 1.73-1.59 (m, 7H), 1.56-1.46 (m, 1H), 1.36-1.21 (m, 3H), 1.20-1.07 (m, 2H) ¹⁹F NMR (377 MHz, METHANOL-d4) δ=−120.74 (br d, J=17.0 Hz, 1F), −122.20-−122.57 (m, 1F).

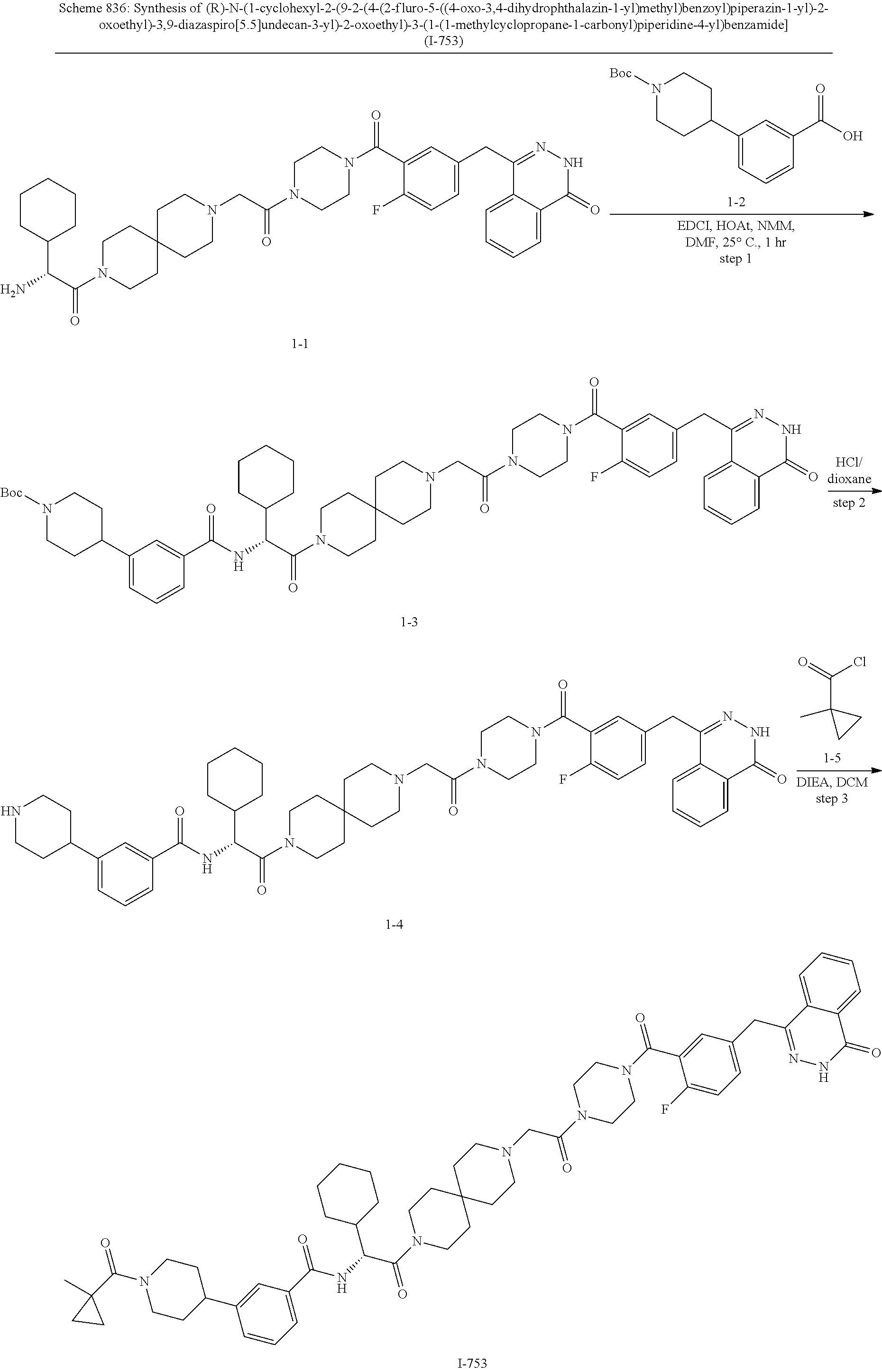

### Step 1: Synthesis of Intermediate 1-3.

To a solution of intermediate 1-1 (2 g, 2.72 mmol, 1 eq, HCl salt) and intermediate 1-2 (829.45 mg, 2.72 mmol, 1 eq) in DMF (20 mL) was added EDCI (520.70 mg, 2.72 mmol, 1 eq) and HOAt (369.71 mg, 2.72 mmol, 379.96 μL, 1 eq) and NMM (274.75 mg, 2.72 mmol, 298.64 μL, 1 eq). The mixture was stirred at 25° C. for 1 hr. To the reaction mixture was added H₂O (50 mL) and then it was extracted with DCM (50 mL*3), the combined organic phase was dried over Na₂SO₄, filtered and concentrated. The residue was purified by flash silica gel chromatography (ISCO; 40 g SepaFlash Silica Flash Column, Eluent of 0˜100% Ethyl acetate/Petroleum ether gradient @50 mL/min) and the eluent was concentrated to give product. Intermediate 1-3 (2 g, 2.03 mmol, 74.59% yield) was obtained as a white solid. LCMS (Method D): Rt: 0.412 min, [M+H]⁺=987.6. SCF: Retention time: 0.780 min, 1.353 min.

### Step 2: Synthesis of Intermediate 1-4.

A solution of intermediate 1-3 (1.3 g, 1.32 mmol, 1 eq) in HCl/dioxane (2 M, 13 mL) was stirred at 25° C. for 1 hr. The reaction mixture was concentrated under reduced pressure to give product. Intermediate 1-4 (1.2 g, 1.23 mmol, 93.31% yield, 94.564% purity, HCl salt) was obtained as a yellow solid. LCMS (Method D): Rt: 0.312 min, [M+H]⁺=887.6. SCF: Retention time: 1.792 min.

### Step 3: Synthesis of I-753

To a solution of intermediate 1-4 (600 mg, 649.66 μmol, 1 eq, HCl salt) and intermediate 1-5 (92.43 mg, 779.60 μmol, 1.2 eq) in DCM (6 mL) was added DIEA (167.93 mg, 1.30 mmol, 226.32 μL, 2 eq). The mixture was stirred at 25° C. for 1 hr. To the reaction mixture was added into H₂O (10 mL) and then it was extracted with DCM (10 mL*3), the combined organic phase was dried over Na₂SO₄, filtered and concentrated. The residue was purified by prep-HPLC (column: CD05-Phenomenex luna C18 150*40*10 um; mobile phase: [water (FA)-ACN]; gradient: 10%-40% B over 10 min), and the eluent was concentrated to remove ACN and lyophilized to give product. I-753 (192.89 mg, 186.37 μmol, 28.69% yield, 98.09% purity, FA salt) was obtained as a white solid. LCMS (Method D): Retention time 0.381 min, [M+H]⁺=969.5. SCF: Retention time: 0.649 min, 1.045 min. ¹H NMR (400 MHz, METHANOL-d4) δ=8.50 (s, 1H), 8.37 (d, J=7.6 Hz, 1H), 7.99-7.92 (m, 1H), 7.92-7.79 (m, 2H), 7.73 (s, 1H), 7.68 (d, J=7.6 Hz, 1H), 7.57-7.31 (m, 4H), 7.17 (t, J=8.8 Hz, 1H), 4.93 (d, J=8.0 Hz, 1H), 4.58 (d, J=13.2 Hz, 2H), 4.39 (s, 2H), 3.87-3.71 (m, 4H), 3.71-3.62 (m, 3H), 3.57-3.42 (m, 5H), 3.34 (s, 2H), 3.03-2.82 (m, 2H), 2.71 (d, J=9.4 Hz, 4H), 1.99-1.83 (m, 4H), 1.76 (s, 2H), 1.74-1.63 (m, 7H), 1.60 (s, 3H), 1.54-1.44 (m, 2H), 1.40-1.17 (m, 7H), 1.16-1.04 (m, 2H), 0.97-0.88 (m, 2H), 0.70-0.58 (m, 2H). ¹⁹F NMR (377 MHz, METHANOL-d₄) δ=−120.74 (d, J=31.1 Hz, 1F).

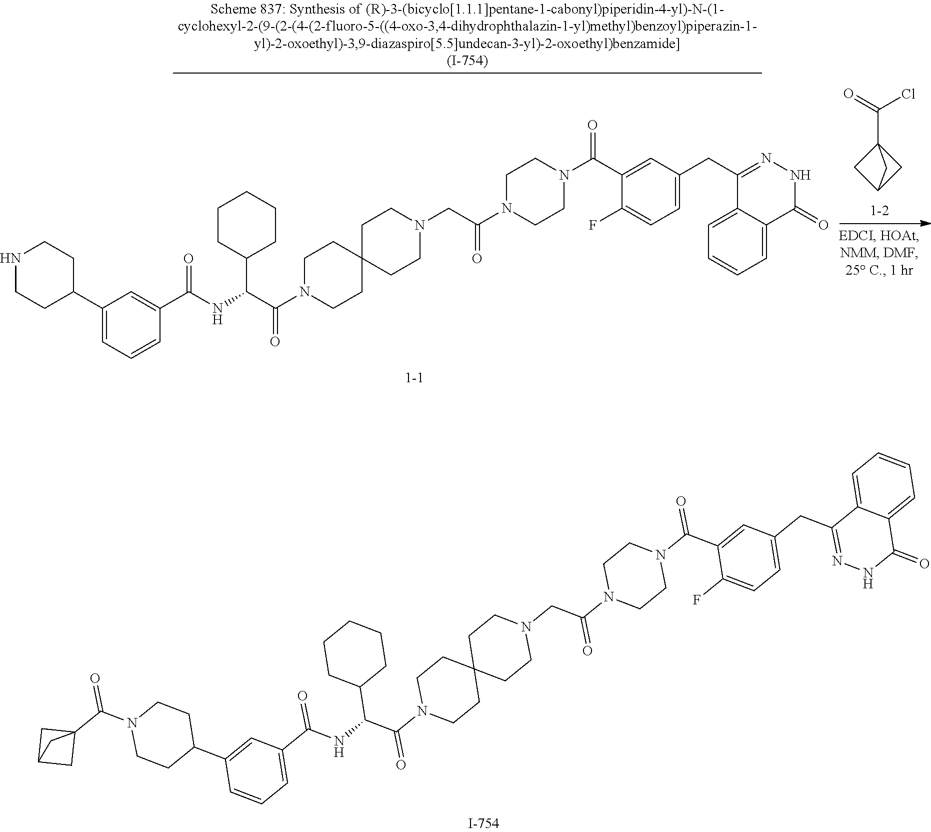 [SMILES: O=C(N[C@@H](C(=O)N1CCC2(CCN(CC(=O)N3CCN(C(=O)c4cc(Cc5n[nH]c(=O)c6ccccc56)ccc4F)CC3)CC2)CC1)C1CCCCC1)c1cccc(C2CCNCC2)c1 ; O=C(Cl)C12CC(C1)C2 ; O=C(N[C@@H](C(=O)N1CCC2(CCN(CC(=O)N3CCN(C(=O)c4cc(Cc5n[nH]c(=O)c6ccccc56)ccc4F)CC3)CC2)CC1)C1CCCCC1)c1cccc(C2CCN(C(=O)C34CC(C3)C4)CC2)c1]

To a solution of intermediate 1-1 (800 mg, 866.22 μmol, 1 eq, HCl salt) and intermediate 1-2 (97.13 mg, 866.22 μmol, 1 eq) in DMF (8 mL) was added EDCI (332.11 mg, 1.73 mmol, 2 eq) and HOAt (117.90 mg, 866.22 μmol, 121.17 μL, 1 eq) and NMM (438.08 mg, 4.33 mmol, 476.17 μL, 5 eq). The mixture was stirred at 25° C. for 1 hr. The reaction mixture was added into H₂O (10 mL) and then extracted with DCM (10 mL*3), the combined organic phase was dried over Na₂SO₄, filtered and concentrated to give a residue. The residue was purified by prep-HPLC (column: CD05-Phenomenex luna C18 150*40*10 um; mobile phase: [water (FA)-ACN]; gradient: 13%-43% B over 10 min), and the eluent was concentrated to remove ACN and lyophilized to give product. I-754 (190.57 mg, 190.30 μmol, 21.97% yield, 97.98% purity) was obtained as a white solid. LCMS (Method D): Retention time: 0.395 min, [M+H]⁺=981.5. SCF: Retention time: 0.675 min, 1.121 min. ¹H NMR (400 MHz, METHANOL-d4) δ=8.48 (s, 1H), 8.37 (d, J=7.6 Hz, 1H), 8.00-7.92 (m, 1H), 7.91-7.80 (m, 2H), 7.75-7.64 (m, 2H), 7.50 (d, J=2.8 Hz, 1H), 7.46-7.33 (m, 3H), 7.17 (t, J=8.8 Hz, 1H), 4.93 (d, J=8.4 Hz, 1H), 4.62 (d, J=13.2 Hz, 1H), 4.48-4.34 (m, 3H), 3.87-3.71 (m, 4H), 3.67 (s, 3H), 3.59 (s, 1H), 3.50 (d, J=5.2 Hz, 4H), 3.34 (s, 2H), 3.20 (s, 1H), 2.97-2.86 (m, 1H), 2.84-2.63 (m, 5H), 2.49 (s, 1H), 2.29-2.12 (m, 6H), 1.98-1.83 (m, 4H), 1.81-1.55 (m, 12H), 1.50 (d, J=4.0 Hz, 2H), 1.37-1.20 (m, 3H), 1.17-1.03 (m, 2H). ¹⁹F NMR (377 MHz, METHANOL-d4) δ=−120.72 (d, J=33.9 Hz, 1F).

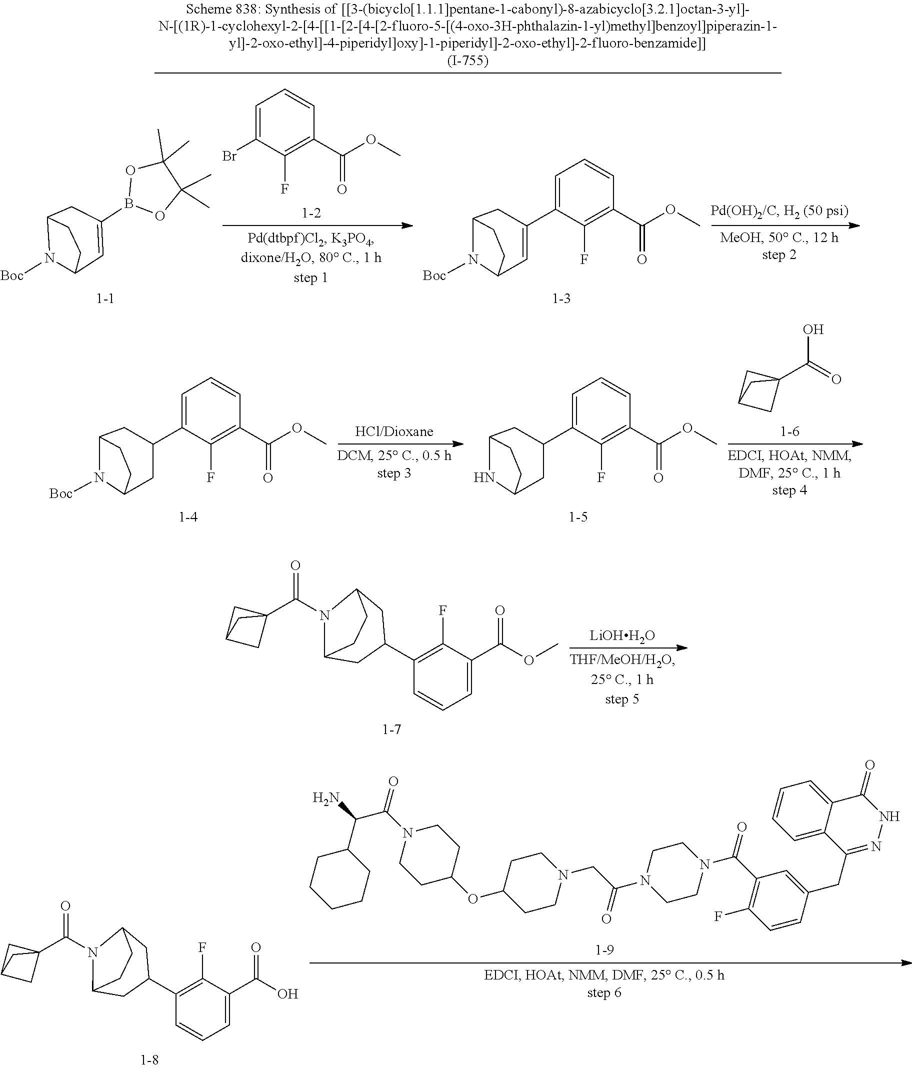

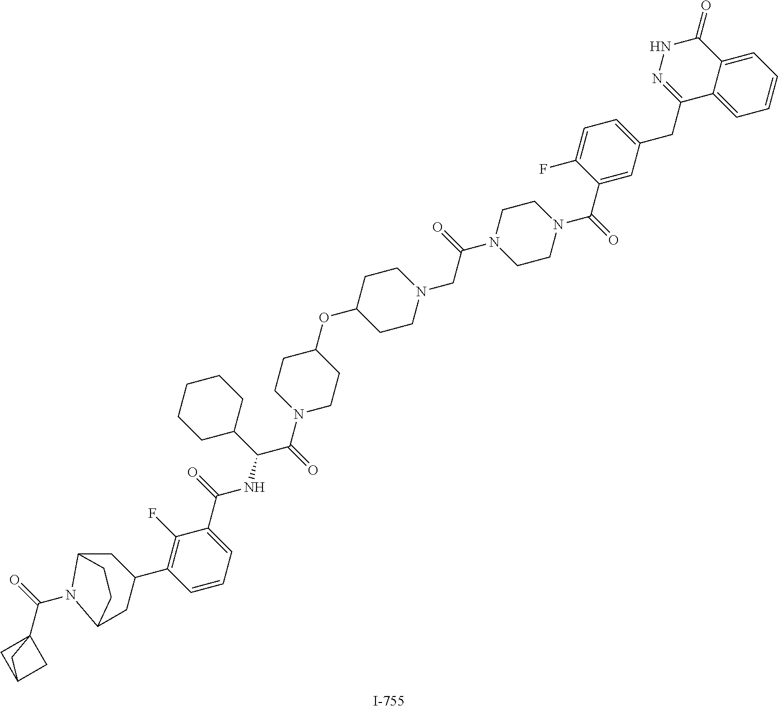 [SMILES: O=C(N[C@@H](C(=O)N1CCC(OC2CCN(CC(=O)N3CCN(C(=O)c4cc(Cc5n[nH]c(=O)c6ccccc56)ccc4F)CC3)CC2)CC1)C1CCCCC1)c1cccc(C2CC3CCC(C2)N3C(=O)C23CC(C2)C3)c1F]

### Step 1: Synthesis of Intermediate 1-3.

A mixture of Intermediate 1-1 (4.45 g, 13.26 mmol, 1.2 eq), Intermediate 1-2 (3 g, 11.6 mmol, 1 eq), Pd(dtbpf)Cl₂ (194.41 mg, 298.29 μmol, 0.1 eq), and K₃PO₄ (1.90 g, 8.95 mmol, 3 eq) in dioxane (50 mL) and H₂O (10 mL) was degassed and purged with N₂ 3 times, and then the mixture was stirred at 80° C. for 1 hr under N₂ atmosphere. The reaction mixture was poured into water (50 mL) and extracted with EA (20 mL*3). The combined organic layers were dried over Na₂SO₄, filtered and concentrated under reduced pressure to give a residue. The residue was purified by flash silica gel chromatography (ISCO; 20 g SepaFlash Silica Flash Column, Eluent of 0˜100% PE/EA@25 mL/min, PE/EA=10:1, Rf=0.3) and the eluent was concentrated under reduced pressure to give product. Intermediate 1-3 (4 g, 11.08 mmol, 92.76% yield) was obtained as a yellow oil. LCMS (Method D): Retention time: 0.487 min, [M-Boc+H]⁺=262.1. ¹H NMR (400 MHz, CHLOROFORM-d) δ=7.83-7.75 (m, 1H), 7.38-7.33 (m, 1H), 7.16-7.13 (m, 1H), 6.23 (d, J=5.2 Hz, 1H), 4.59-4.27 (m, 2H), 3.93 (s, 3H), 3.09 (br s, 1H), 2.28-2.13 (m, 2H), 2.05-1.98 (m, 2H), 1.82 (br s, 1H), 1.48 (s, 9H).

### Step 2: Synthesis of Intermediate 1-4.

To a solution of intermediate 1-3 (4 g, 11.07 mmol, 1 eq) in MeOH (80 mL) was added Pd(OH)₂/C (500 mg, 20% purity, 1.00 eq) under Ar atmosphere. The suspension was degassed and purged with H₂ 3 times. The mixture was stirred under H₂ (50 Psi) at 50° C. for 12 hrs. The mixture was filtered and concentrated under reduced pressure to give product. Intermediate 1-4 (4 g, crude) was obtained as a yellow oil. LCMS (Method D): Retention time: 0.497 min, [M-tBu+H]⁺=308.1. SCF: Retention time: 0.937, 1.065 min. ¹H NMR (400 MHz, CHLOROFORM-d) δ=7.71-7.64 (m, 1H), 7.34-7.26 (m, 1H), 7.10-7.03 (m, 1H), 4.33-4.13 (m, 2H), 3.85 (s, 3H), 3.55-2.76 (m, 1H), 2.49-2.31 (m, 1H), 2.02-1.95 (m, 2H), 1.81 (s, 1H), 1.75 (d, J=7.6 Hz, 1H), 1.61-1.54 (m, 2H), 1.43 (d, J=3.6 Hz, 9H), 1.39 (s, 1H).

### Step 3: Synthesis of Intermediate 1-5.

To a solution of intermediate 1-4 (2.00 g, 5.50 mmol, 1 eq) in DCM (5 mL) was added HCl/dioxane (2 M, 20 mL, 7.27 eq). The mixture was stirred at 25° C. for 0.5 hr. The mixture was concentrated under reduced pressure to give product. Intermediate 1-5 (2 g, crude, HCl salt) was obtained as a yellow oil. LCMS (Method D): Retention time: 0.248 min, [M+H]⁺=264.1.

### Step 4: Synthesis of Intermediate 1-7.

To a solution of intermediate 1-6 (329.16 mg, 2.94 mmol, 1.1 eq) in DMF (10 mL) was added EDCI (1.53 g, 8.01 mmol, 3 eq), HOAt (363.24 mg, 2.67 mmol, 373.32 μL, 1 eq), NMM (1.35 g, 13.34 mmol, 1.47 mL, 5 eq) and intermediate 1-5 (800 mg, 2.67 mmol, 1 eq, HCl salt). The mixture was stirred at 25° C. for 1 hr. The reaction mixture was poured into water (30 mL) and extracted with EA (20 mL*3). The combined organic layers were dried over Na₂SO₄, filtered and concentrated under reduced pressure to give a residue. The residue was purified by flash silica gel chromatography (ISCO; 20 g SepaFlash Silica Flash Column, Eluent of 0˜100% PE/EA @50 mL/min, PE/EA=2:1, Rf=0.4), and the eluent was concentrated under reduced pressure to give product. Intermediate 1-7 (910 mg, 2.53 mmol, 94.89% yield, 99.463% purity) as a yellow oil. LCMS (Method D): Retention time: 0.428 min, [M+H]⁺=358.4. ¹HNMR (400 MHz, CHLOROFORM-d) δ=7.79-7.71 (m, 1H), 7.42-7.33 (m, 1H), 7.17-7.09 (m, 1H), 4.81-4.71 (m, 1H), 4.59-4.47 (m, 1H), 3.92 (d, J=1.2 Hz, 3H), 3.67-3.53 (m, 1H), 2.48 (d, J=6.0 Hz, 1H), 2.23-2.12 (m, 6H), 2.01 (d, J=3.6 Hz, 4H), 1.85-1.77 (m, 1H), 1.77-1.63 (m, 2H), 1.61-1.51 (m, 1H).

### Step 5: Synthesis of Intermediate 1-8.

To a solution of intermediate 1-7 (800 mg, 2.24 mmol, 1 eq) in THF (3 mL), MeOH (3 mL) and H₂O (3 mL) was added LiOH·H₂O (281.78 mg, 6.71 mmol, 3 eq). The mixture was stirred at 25° C. for 1 hr. The reaction mixture was adjusted to pH=3 with 1M aq. HCl, diluted with H₂O (10 mL) and then extracted with EA (10 mL*3), dried over Na2SO4, filtered and concentrated to give product. Intermediate 1-8 (750 mg, crude) was obtained as a white solid. LCMS (Method D): Retention time: 0.384 min, [M+H]⁺=344.1.

### Step 6: Synthesis of I-755

To a solution of intermediate 1-8 (250 mg, 728.03 μmol, 1 eq) in DMF (5 mL) was added EDCI (418.69 mg, 2.18 mmol, 3 eq), HOAt (99.09 mg, 728.03 μmol, 101.84 μL, 1 eq), NMM (368.19 mg, 3.64 mmol, 400.21 μL, 5 eq) and intermediate 1-9 (557.92 mg, 728.03 μmol, 1 eq, HCl salt). The mixture was stirred at 25° C. for 0.5 hr. The reaction mixture was poured into water (15 mL) and extracted with EA (10 mL*3). The combined organic layers were dried over Na₂SO₄, filtered and concentrated under reduced pressure to give a residue. The residue was purified by reverse phase chromatography (0.1% FA) and the eluent was lyophilized to give product. I-755 (394.86 mg, 357.18 μmol, 49.06% yield, 99.62% purity, FA salt) was obtained as a white solid. LCMS (Method D): Retention time: 0.458 min, [M+H]⁺=1055.8. SCF: Retention time: 3.835 min, 4.992 min. ¹H NMR (400 MHz, METHANOL-d4) δ=8.43 (s, 1H), 8.37 (d, J=7.6 Hz, 1H), 7.95 (d, J=6.4 Hz, 1H), 7.91-7.77 (m, 2H), 7.61-7.32 (m, 4H), 7.27-7.10 (m, 2H), 4.95 (d, J=7.2 Hz, 1H), 4.71-4.61 (m, 2H), 4.39 (s, 2H), 4.12-4.00 (m, 1H), 4.00-3.84 (m, 1H), 3.79 (d, J=9.6 Hz, 5H), 3.71-3.66 (m, 2H), 3.63 (s, 2H), 3.56-3.44 (m, 3H), 3.35 (s, 2H), 3.29-3.17 (m, 1H), 3.16-3.03 (m, 2H), 2.87-2.72 (m, 2H), 2.52-2.47 (m, 1H), 2.45 (d, J=6.8 Hz, 1H), 2.29-2.17 (m, 6H), 2.16-2.06 (m, 1H), 2.06-1.93 (m, 5H), 1.92-1.73 (m, 10H), 1.72-1.47 (m, 5H), 1.34-1.08 (m, 5H). ¹⁹FNMR (400 MHz, METHANOL-d₄) δ=−120.756, −121.686.

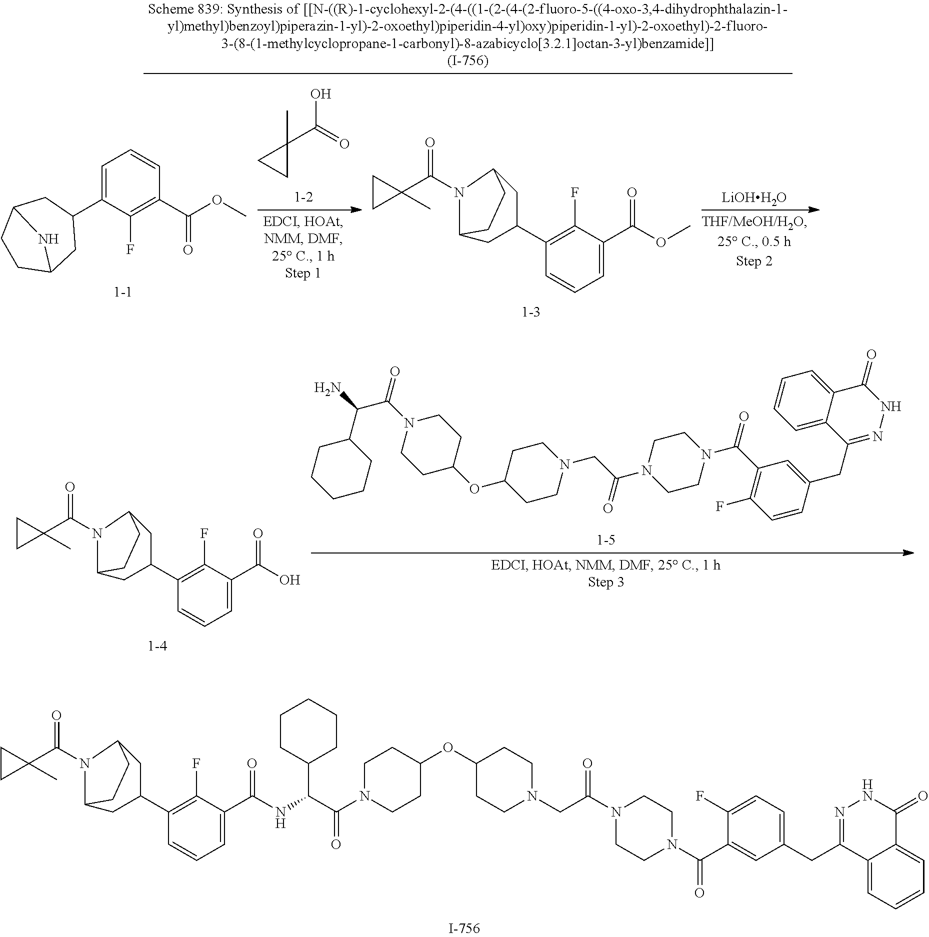

### Step 1: Synthesis of Intermediate 1-3.

To a solution of intermediate 1-2 (320.62 mg, 3.20 mmol, 1.2 eq) in DMF (10 mL) was added EDCI (1.53 g, 8.01 mmol, 3 eq), HOAt (363.24 mg, 2.67 mmol, 373.32 μL, 1 eq), NMM (1.35 g, 13.34 mmol, 1.47 mL, 5 eq) and intermediate 1-1 (800 mg, 2.67 mmol, 1 eq, HCl salt). The mixture was stirred at 25° C. for 0.5 hr. The reaction mixture was combined with another lot of material for work-up, and the mixture was poured into water (30 mL) and extracted with EA (20 mL*3). The combined organic layers were dried over Na₂SO₄, filtered and concentrated under reduced pressure to give a residue. The residue was purified by flash silica gel chromatography (ISCO; 20 g SepaFlash Silica Flash Column, Eluent of 0˜100% PE/EA) @50 mL/min, PE/EA=2:1, Rf=0.4) and the eluent was concentrated under reduced pressure to give product. Intermediate 1-3 (820 mg, 2.35 mmol, 88.20% yield, 99.154% purity) was obtained as a white solid. LCMS (Method D): Retention time: 0.429 min, [M+H]⁺=346.1.

### Step 2: Synthesis of Intermediate 1-4.

To a solution of intermediate 1-3 (800 mg, 2.32 mmol, 1 eq) in MeOH (3 mL), THF (3 mL) and H₂O (3 mL) was added LiOH·H₂O (291.58 mg, 6.95 mmol, 3 eq). The mixture was stirred at 25° C. for 0.5 hr. The reaction mixture was combined with another lot of material for work-up. The reaction was adjusted to pH=3 with 1M aq. HCl, and then diluted with H₂O (10 mL) and extracted with EA (10 mL*3), dried over Na₂SO₄, filtered and concentrated to give product. Intermediate 1-4 (750 mg, crude) was obtained as a white solid. LCMS (method D): Retention time: 0.340 min, [M+H]⁺=332.2.

### Step 3: Synthesis of I-756

To a solution of intermediate 1-4 (200 mg, 603.54 μmol, 1 eq) in DMF (5 mL) was added EDCI (347.10 mg, 1.81 mmol, 3 eq), HOAt (82.15 mg, 603.54 μmol, 84.43 μL, 1 eq), NMM (305.23 mg, 3.02 mmol, 331.77 μL, 5 eq) and intermediate 1-5 (462.51 mg, 603.54 μmol, 1 eq, HCl salt). The mixture was stirred at 25° C. for 1 hr. The reaction mixture was combined with another lot of material for work-up, the mixture was poured into water (15 mL) and extracted with EA (10 mL*3). The combined organic layers were dried over Na₂SO₄, filtered and concentrated under reduced pressure to give a residue. The residue was purified by reverse phase chromatography (0.1% FA) and the eluent was lyophilized to give the product. I-756 (175.2 mg, 160.65 μmol, 26.62% yield, 99.88% purity, FA salt) was obtained as a white solid. LCMS (Method D): Retention time: 0.395 min, [M+H]⁺=1043.6. SCF: Retention time: 1.732, 2.533, 3.484 min. ¹H NMR (400 MHz, METHANOL-d4) δ=8.42 (s, 1H), 8.37 (d, J=7.6 Hz, 1H), 7.94 (d, J=6.4 Hz, 1H), 7.92-7.78 (m, 2H), 7.58-7.42 (m, 3H), 7.37 (t, J=7.2 Hz, 1H), 7.27-7.12 (m, 2H), 4.96 (d, J=7.2 Hz, 1H), 4.79-4.57 (m, 2H), 4.39 (s, 2H), 4.09-3.84 (m, 2H), 3.83-3.66 (m, 7H), 3.62 (br s, 2H), 3.55-3.41 (m, 3H), 3.35 (s, 1H), 3.27 (d, J=3.6 Hz, 1H), 3.19-3.05 (m, 2H), 2.91-2.76 (m, 2H), 2.60-2.38 (m, 1H), 2.18-2.03 (m, 2H), 1.99 (s, 6H), 1.89-1.72 (m, 9H), 1.69 (d, J=11.6 Hz, 4H), 1.57-1.44 (m, 1H), 1.43-1.35 (m, 3H), 1.34-1.19 (m, 3H), 1.12 (d, J=10.4 Hz, 2H), 1.04-0.81 (m, 2H), 0.64 (d, J=10.8 Hz, 2H). ¹⁹FNMR (400 MHz, METHANOL-d4) δ=−120.749, −122.175.

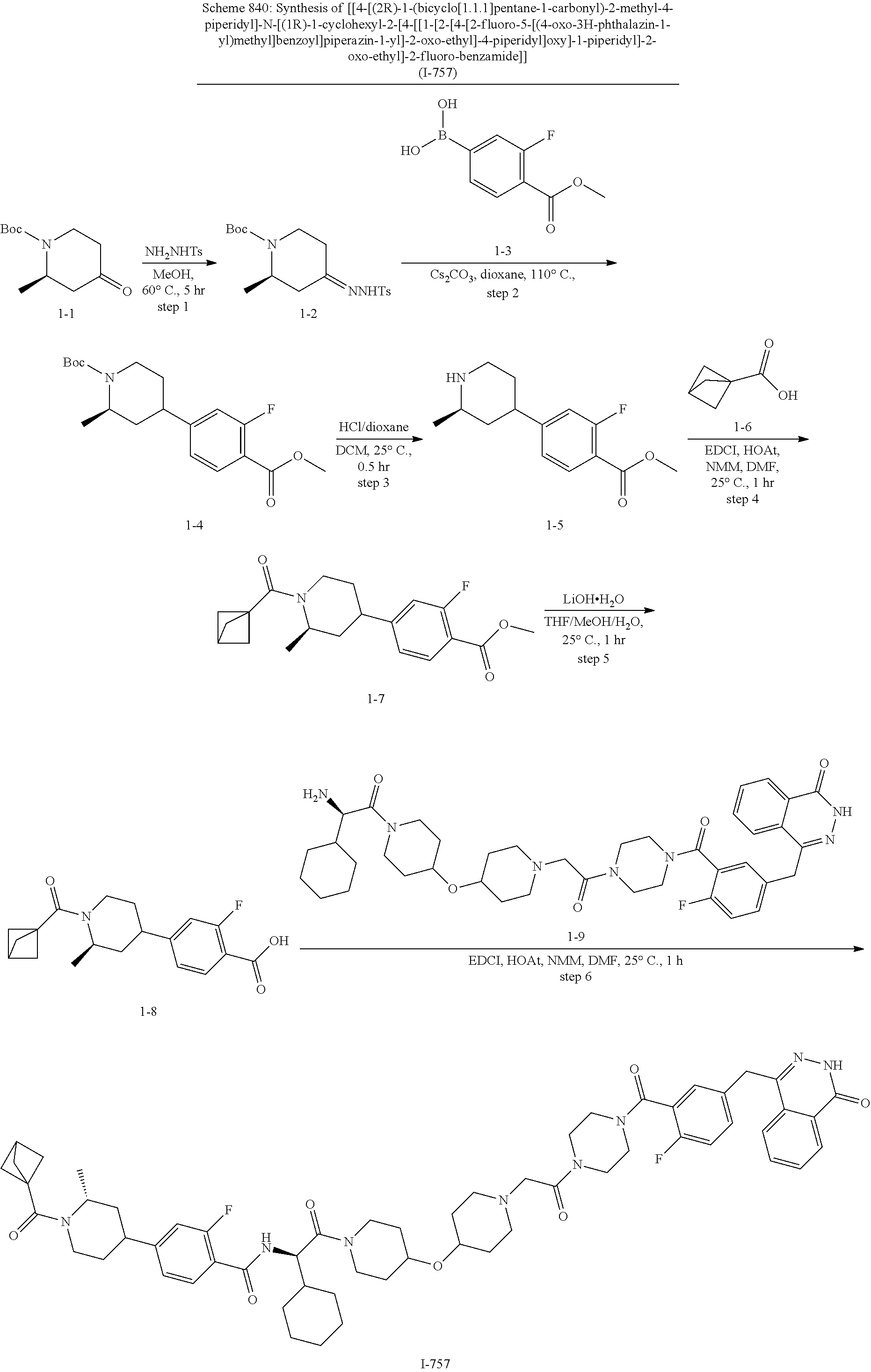

### Step 1: Synthesis of Intermediate 1-3.

To a solution of NH₂NHTs (174.64 mg, 937.77 μmol, 1 eq) in MeOH (3 mL) was added intermediate 1-1 (200 mg, 937.77 μmol, 1 eq). The mixture was stirred at 60° C. for 5 hrs. The reaction mixture was concentrated in vacuum to give product. Intermediate 1-2 (300 mg, 786.39 μmol, 83.86% yield) was obtained as a white solid. LCMS (Method D): Rt=0.417 min, [M+H]⁺=382.0.

### Step 2: Synthesis of Intermediate 1-4.

To a solution of intermediate 1-2 (250 mg, 655.33 μmol, 1 eq) and intermediate 1-3 (129.73 mg, 655.33 μmol, 1 eq) in dioxane (4 mL) was added Cs₂CO₃ (320.28 mg, 982.99 μmol, 1.5 eq). The mixture was stirred at 110° C. for 12 hrs. The mixture was washed with water (5 mL) and extracted with EA (5 mL*3). The combined organic layers dried over sodium sulfate, filtered and concentrated under reduced pressure to give a residue. The residue was purified by flash silica gel chromatography (ISCO; 20 g SepaFlash Silica Flash Column, Eluent of 0˜20% EA/PE @60 mL/min) and then the eluent was concentrated in vacuum to give product. Intermediate 1-4 (65 mg, 177.57 μmol, 27.10% yield, 96% purity) was obtained as colorless oil. LCMS (Method D): Rt=0.494 min, [M-tBu+H]⁺=296.0. SCF: Rt=0.650 min, 0.725 min. ¹H NMR (400 MHz, CHLOROFORM-d) δ=7.88-7.86 (m, 1H), 7.05 (d, J=8.8 Hz, 1H), 7.01-6.97 (m, 1H), 4.01-3.95 (m, 1H), 3.93 (s, 3H), 3.83-3.82 (m, 1H), 3.28-3.19 (m, 1H), 2.87-2.75 (m, 1H), 2.25-2.10 (m, 1H), 1.94-1.93 (m, 1H), 1.80-1.79 (m, 1H), 1.64-1.60 (m, 1H), 1.50-1.49 (m, 9H), 1.21 (d, J=6.4 Hz, 3H). ¹⁹F NMR (376 MHz, CHLOROFORM-d) δ=−109.336.

### Step 3: Synthesis of Intermediate 1-5

To a solution of intermediate 1-4 (60 mg, 170.74 μmol, 1 eq) in DCM (0.5 mL) was added HCl/dioxane (2 M, 0.5 mL). The mixture was stirred at 25° C. for 0.5 hr. The reaction mixture was concentrated in vacuum to give product. Intermediate 1-5 (60 mg, crude, HCl salt) was obtained as a white solid. LCMS (Method D): Rt=0.234 min, [M+H]⁺=252.0. SCF: Rt=1.285 min, 1.422 min. ¹H NMR (400 MHz, METHANOL-d4) δ=7.94-7.88 (m, 1H), 7.26-7.14 (m, 2H), 3.90 (s, 3H), 3.56-3.48 (m, 1H), 3.43-3.34 (m, 1H), 3.26-3.13 (m, 1H), 3.05-3.04 (m, 1H), 2.18-2.06 (m, 2H), 2.04-1.92 (m, 1H), 1.90-1.79 (m, 1H), 1.39 (d, J=6.4 Hz, 3H). ¹⁹F NMR (376 MHz, METHANOL-d4) δ=−111.066.

### Step 4: Synthesis of Intermediate 1-7

To a solution of intermediate 1-6 (19.48 mg, 173.76 μmol, 1 eq) in DMF (0.5 mL) was added EDCI (99.93 mg, 521.27 μmol, 3 eq), HOAt (23.65 mg, 173.76 μmol, 24.31 μL, 1 eq), and NMM (87.88 mg, 868.79 μmol, 95.52 μL, 5 eq). Then intermediate 1-5 (50 mg, 173.76 μmol, 1 eq, HCl salt) was added. The mixture was stirred at 25° C. for 1 hr. The mixture was washed with water (1 mL) and extracted with EA (2 mL*3). The combined organic layers dried over sodium sulfate, filtered and concentrated under reduced pressure to give a residue. The residue was purified by flash silica gel chromatography (ISCO; 4 g SepaFlash Silica Flash Column, Eluent of 0˜60% EA/PE @20 mL/min) and then the eluent was concentrated in vacuum to give product. Intermediate 1-7 (40 mg, 114.65 μmol, 65.98% yield, 99% purity) was obtained as a white solid. LCMS (Method D): Rt=0.449 min, [M+H]⁺=346.2. ¹H NMR (400 MHz, METHANOL-d4) δ=7.86-7.84 (m, 1H), 7.21-7.11 (m, 2H), 4.41-3.99 (m, 2H), 3.89 (s, 3H), 3.40-3.33 (m, 1H), 3.21-3.10 (m, 1H), 3.00-2.71 (m, 1H), 2.51-2.45 (m, 1H), 2.26-2.13 (m, 7H), 2.00-1.87 (m, 1H), 1.86-1.78 (m, 1H), 1.77-1.66 (m, 1H), 1.65-1.48 (m, 1H), 1.29-1.15 (m, 3H). ¹⁹F NMR (376 MHz, METHANOL-d4) δ=−111.397.

### Step 5: Synthesis of Intermediate 1-8

To a solution of intermediate 1-7 (40 mg, 115.81 μmol, 1 eq) in THF (0.2 mL), MeOH (0.2 mL) and H₂O (0.2 mL) was added LiOH·H₂O (14.58 mg, 347.42 μmol, 3 eq). The mixture was stirred at 25° C. for 1 hr. The pH was acidified to 5-6 with 1N HCl, and the mixture was washed with water (3 mL) and extracted with EA (4 mL*3). The combined organic layers were dried over sodium sulfate, filtered and concentrated under reduced pressure to give product. Intermediate 1-8 (35 mg, 105.62 μmol, 91.20% yield) was obtained as a white solid. LCMS (Method D): Rt=0.393 min, [M+H]⁺=332.1.

### Step 6: Synthesis of I-757

To a solution of intermediate 1-8 (35 mg, 105.62 μmol, 1 eq) in DMF (1 mL) was added EDCI (60.74 mg, 316.86 μmol, 3 eq), HOAt (14.38 mg, 105.62 μmol, 14.77 μL, 1 eq) and NMM (53.42 mg, 528.09 μmol, 58.06 μL, 5 eq). Then intermediate 1-9 (80.94 mg, 105.62 μmol, 1 eq, HCl salt) was added. The mixture was stirred at 25° C. for 1 hr. The mixture was washed with water (3 mL) and extracted with EA (4 mL*3). The combined organic layers were dried over sodium sulfate, filtered and concentrated under reduced pressure to give a residue. The residue was purified by reversed phase chromatography (0.1% FA condition), the eluent was concentrated under reduced pressure to remove ACN and lyophilized to give product. I-757 (44.4 mg, 42.08 μmol, 39.84% yield, 98.872% purity) was obtained as a white solid. LCMS (Method D): Rt=0.417 min, [M+H]⁺=1043.5. SCF: Rt=1.038 min, 1.441 min. ¹H NMR (400 MHz, METHANOL-d4) δ=8.52 (s, 1H), 8.37 (d, J=7.6 Hz, 1H), 7.98-7.92 (m, 1H), 7.90-7.82 (m, 2H), 7.71-7.70 (m, 1H), 7.48 (d, J=4.4 Hz, 1H), 7.37 (d, J=4.0 Hz, 1H), 7.22-7.14 (m, 3H), 4.98 (d, J=7.2 Hz, 1H), 4.39 (s, 2H), 4.35-4.25 (m, 1H), 4.13-3.95 (m, 2H), 3.93-3.83 (m, 1H), 3.79 (s, 2H), 3.73-3.71 (m, 2H), 3.67 (s, 1H), 3.53 (d, J=13.2 Hz, 4H), 3.50-3.39 (m, 2H), 3.27 (s, 2H), 2.89-2.71 (m, 3H), 2.50-2.45 (m, 1H), 2.42-2.28 (m, 2H), 2.24-2.22 (m, 2H), 2.20 (s, 3H), 2.17 (d, J=1.6 Hz, 1H), 1.98-1.87 (m, 4H), 1.87-1.74 (m, 8H), 1.68 (d, J=5.6 Hz, 4H), 1.61-1.60 (m, 2H), 1.56-1.50 (m, 1H), 1.40-1.22 (m, 4H), 1.19 (d, J=6.4 Hz, 3H), 1.15-1.08 (m, 2H). ¹⁹F NMR (376 MHz, METHANOL-d4) δ=−115.338, −120.794.

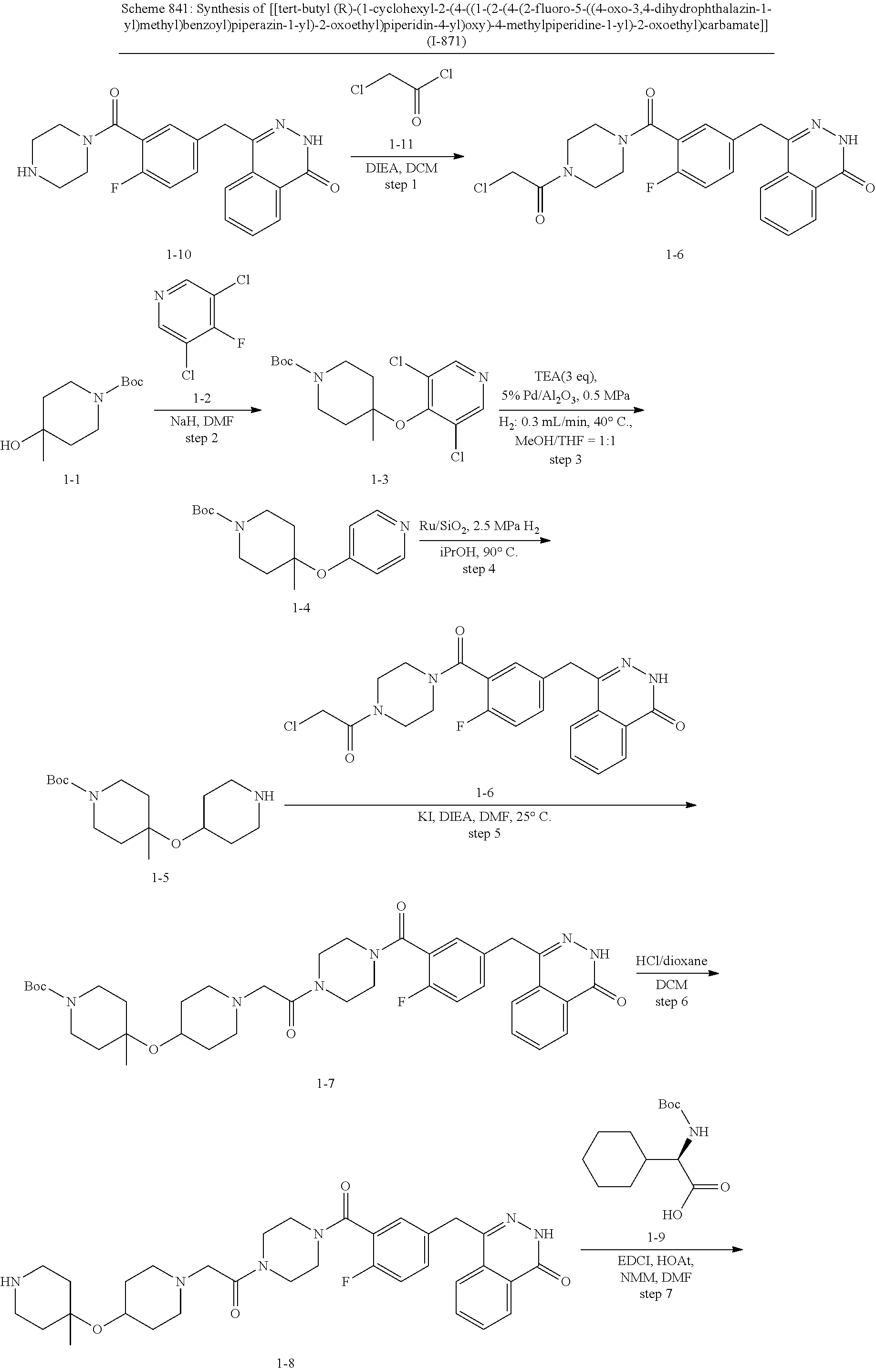

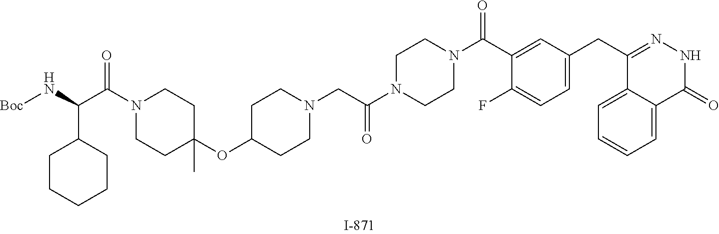 [SMILES: CC(C)(C)OC(=O)N[C@@H](C(=O)N1CCC(C)(OC2CCN(CC(=O)N3CCN(C(=O)c4cc(Cc5n[nH]c(=O)c6ccccc56)ccc4F)CC3)CC2)CC1)C1CCCCC1]

### Step 1: Synthesis of Intermediate 1-6

To a solution of Intermediate 1-10 (10 g, 27.29 mmol, 1 eq) in DCM (100 mL) was added DIEA (10.58 g, 81.88 mmol, 14.26 mL, 3 eq) and Intermediate 1-11 (6.17 g, 54.59 mmol, 4.35 mL, 2 eq). The mixture was stirred at 0-25° C. for 1 h. The reaction was poured into water (200 ml) and extracted with DCM (100 ml*3). The combined organic layers were washed with sat. NaHCO₃ (100 ml*3), dried over anhydrous Na₂SO₄, filtered and concentrated under vacuum to give a residue. The residue was purified by prep-HPLC (column: Kromasil Eternity XT 250*80 mm*10 um; mobile phase: [water (ammonia hydroxide v/v)-ACN]; gradient: 20%-45% B over 20 min) and concentrated under vacuum to remove ACN. The aqueous was extracted with DCM (300 ml*3), the combined organic layers were dried over anhydrous Na₂SO₄, filtered and concentrated under vacuum. Intermediate 1-6 (7.5 g, 14.60 mmol, 53.48% yield, 86.19% purity) was obtained as a gray solid. LCMS (Method G): Retention time: 0.470 min, [M+H]=443.2. ¹H NMR (400 MHz, DMSO-d6) δ=12.60 (s, 1H), 8.26 (dd, J=1.0, 7.8 Hz, 1H), 7.99-7.94 (m, 1H), 7.93-7.80 (m, 2H), 7.51-7.35 (m, 2H), 7.23 (t, J=9.0 Hz, 1H), 4.45-4.29 (m, 4H), 3.73-3.52 (m, 4H), 3.39 (br s, 2H), 3.21 (br d, J=16.1 Hz, 2H). ¹⁹F NMR (377 MHz, DMSO-d6) δ=−119.700.

### Step 2: Synthesis of Intermediate 1-3

To a solution of Intermediate 1-1 (8.7 g, 40.41 mmol, 1 eq) in DMF (70 mL) was added NaH (3.23 g, 80.82 mmol, 60% purity, 2 eq) slowly at 0° C. After addition, the mixture was warmed to 25° C. and stirred at this temperature for 1 hr, and then Intermediate 1-2 (10.06 g, 60.62 mmol, 1.5 eq) in DMF (20 mL) was added at 25° C. The resulting mixture was stirred at 25° C. for 2 hr. The reaction mixture was quenched by saturated NH₄Cl aq. (100 mL) and extracted with EA (50 mL*3). The combined organic layers were washed with brine (50 mL*3), dried over anhydrous Na₂SO₄, filtered and concentrated under reduced pressure to give a residue. The residue was purified by flash silica gel chromatography (ISCO; 120 g SepaFlash Silica Flash Column, Eluent of 0˜20% Ethylacetate/Petroleum ethergradient @100 mL/min) and concentrated under vacuum. Intermediate 1-3 (9.5 g, 26.30 mmol, 65.07% yield) was obtained as white solid. ¹H NMR (400 MHz, CHLOROFORM-d) δ=8.45 (s, 2H), 3.82 (br d, J=12.0 Hz, 2H), 3.55 (br t, J=11.9 Hz, 2H), 2.38 (br d, J=13.3 Hz, 2H), 1.75-1.61 (m, 2H), 1.48 (s, 9H), 1.25 (s, 3H).

### Step 2: Synthesis of Intermediate 1-4

A solution of Intermediate 1-3 (8.5 g, 23.53 mmol, 1 eq) and TEA (2.38 g, 23.53 mmol, 3.27 mL, 1 eq) in MeOH (85 mL) was stirred at 25° C. until a clear solution form. A fixed bed (volume 20 mL) was completely packed with granular catalyst 5% Pd/Al₂O₃ (1.00 eq, 10 g). The H₂ back pressure regulator was adjusted to 0.5 MPa, and the flow rate of H₂ was set to 60 mL/min. Then the solution was pumped by (1.212 mL/min) into the fixed bed (9.525(½″) mm, 4 mL, 55° C.). The solution was flowing through the fixed bed, and then the reaction mixture was collected from the reactor output. After the reaction was finished, the tubing was washed with MeOH (100 mL). The reaction solution for was concentrated under vacuum to give Intermediate 1-4 (7.6 g, crude) as a white solid. ¹H NMR (400 MHz, DMSO-d6) δ=8.45-8.38 (m, 2H), 7.10-7.05 (m, 2H), 3.69-3.61 (m, 2H), 3.22-3.13 (m, 2H), 2.02 (brd, J=13.8 Hz, 2H), 1.70-1.63 (m, 2H), 1.49 (s, 3H), 1.45-1.43 (m, 9H).

### Step 3: Synthesis of Intermediate 1-5

A solution of Intermediate 1-4 (7.6 g, 25.99 mmol, 1 eq) in IPA (76 mL) and THF (114 mL) was dissolved to form a clear solution. A fixed bed (volume 20 mL) was packed with granular catalyst (Ru/SiO₂ (10 g, 10% purity)). The H₂ back pressure regulator was adjusted to 2.5 MPa and the flow rate of H₂ was set to 60 mL/min. Then the solution was pumped by (1 mL/min) to the fixed bed (12.7 mm, 4 mL, 100° C.). The solution flowed through the fixed bed, and then the reaction mixture was collected from the reactor output. After the reaction fluid injection was complete, the fixed bed was washed by extra IPA (114 mL). The crude was used for the next step directly without purification. Intermediate 1-5 (7 g, crude) was obtained as a white solid. ¹H NMR (400 MHz, DMSO-d6) δ=3.88-3.77 (m, 1H), 3.52 (br d, J=13.0 Hz, 2H), 3.15-3.02 (m, 4H), 2.99-2.92 (m, 2H), 1.89-1.77 (m, 2H), 1.72-1.48 (m, 5H), 1.38 (s, 11H), 1.14 (s, 3H).

### Step 4: Synthesis of Intermediate 1-7

To a solution of Intermediate 1-5 (0.8 g, 2.68 mmol, 1 eq) in DMF (8 mL) was added Intermediate 1-6 (1.19 g, 2.68 mmol, 1 eq), DIEA (1.39 g, 10.72 mmol, 1.87 mL, 4 eq) and KI (1.78 g, 10.72 mmol, 4 eq). The mixture was stirred at 25° C. for 1 hr. The mixture was filtered. The filtrate was purified by reversed-phase chromatography (0.1% NH₃·H₂O). Intermediate 1-7 (1.3 g, 1.81 mmol, 67.59% yield, 98.237% purity) was obtained as a light yellow solid. LCMS (Method G): Retention time: 0.642 min, [M+H]⁺=705.5.

### Step 6: Synthesis of Intermediate 1-8

To a solution of Intermediate 1-7 (1 g, 1.42 mmol, 1 eq) in DCM (5 mL) was added HCl/dioxane (4 M, 5.00 mL, 14.10 eq). The mixture was stirred at 25° C. for 0.5 h. The mixture was concentrated under vacuum to give Intermediate 1-8 (0.9 g, crude, HCl salt) as a white solid. LCMS (Method G): Retention time: 0.775 min, [M+H]⁺=605.3.

### Step 7: Synthesis of I-871

To a solution of Intermediate 1-9 (361.20 mg, 1.40 mmol, 1 eq) in DMF (10 mL) was added HOAt (191.06 mg, 1.40 mmol, 196.36 μL, 1 eq), EDCI (538.17 mg, 2.81 mmol, 2 eq), NMM (709.89 mg, 7.02 mmol, 771.62 μL, 5 eq) and Intermediate 1-8 (0.9 g, 1.40 mmol, 1 eq, HCl salt). The mixture was stirred at 25° C. for 1 h. The mixture was poured onto H₂O (30 mL), filtered and the filter cake was washed with H₂O (10 mL*3) and concentrated under vacuum to give I-871 (1.1 g, crude). Then 150 mg of the crude was dissolved in MeOH (2 mL) and it was purified by prep-HPLC (column: Waters Xbridge 150*25 mm*5 um; mobile phase: [water(NH₄HCO₃)-ACN]; gradient: 38%-68% B over 15 min). I-872 (89.39 mg, 104.64 μmol, 58.88% yield, 98.80% purity) was obtained as a white solid. LCMS (Method G): Retention time=0.685 min, [M+H]⁺=844.6. SCF: Retention time=1.817 min. ¹H NMR (400 MHz, DMSO-d6) δ=12.60 (s, 1H), 8.26 (d, J=7.6 Hz, 1H), 8.01-7.94 (m, 1H), 7.91-7.80 (m, 2H), 7.46-7.35 (m, 2H), 7.25-7.23 (m, 1H), 6.75-6.73 (m, 1H), 4.33 (s, 2H), 4.21 (br d, J=6.4 Hz, 1H), 3.98-3.44 (m, 9H), 3.20-2.99 (m, 5H), 2.67 (br d, J=2.0 Hz, 2H), 2.17-2.05 (m, 2H), 1.70-1.55 (m, 9H), 1.50-1.26 (m, 15H), 1.14 (br d, J=15.2 Hz, 6H), 0.98-0.87 (m, 2H). ¹⁹F NMR (377 MHz, DMSO-d6) δ=−118.06-−121.37 (m, 1F).

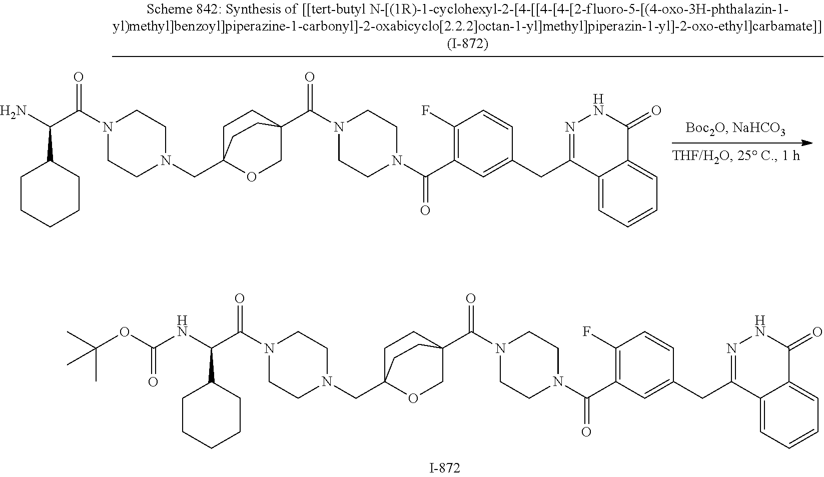 [SMILES: N[C@@H](C(=O)N1CCN(CC23CCC(C(=O)N4CCN(C(=O)c5cc(Cc6n[nH]c(=O)c7ccccc67)ccc5F)CC4)(CC2)CO3)CC1)C1CCCCC1 ; CC(C)(C)OC(=O)N[C@@H](C(=O)N1CCN(CC23CCC(C(=O)N4CCN(C(=O)c5cc(Cc6n[nH]c(=O)c7ccccc67)ccc5F)CC4)(CC2)CO3)CC1)C1CCCCC1]

To a solution of Intermediate 1-1 (150 mg, 192.71 μmol, 1 eq, HCl salt) in THF (1 mL) and H₂O (1 mL) was added NaHCO₃ (48.57 mg, 578.14 μmol, 22.50 μL, 3 eq) and (Boc)₂O (63.09 mg, 289.07 μmol, 66.41 μL, 1.5 eq). The mixture was stirred at 25° C. for 1 hr. The residue was diluted with H₂O (5 mL) and extracted with EA (5 mL*3), dried over Na₂SO₄, filtered and concentrated under reduced pressure to give a residue. The crude product was purified by reversed-phase chromatography (0.1% NH₄HCO₃ condition). The eluent was concentrated under reduced pressure to remove ACN, then lyophilized to afford product. I-872 (24 mg, 28.22 μmol, 14.64% yield, 99% purity) was obtained as a white solid. LCMS (Method D): Rt=0.384 min, [M+H]⁺=842.5. SCF: Rt=0.909 min. ¹H NMR (400 MHz, METHANOL-d4) δ=8.40-8.33 (m, 1H), 7.98-7.92 (m, 1H), 7.91-7.80 (m, 2H), 7.54-7.45 (m, 1H), 7.37-7.35 (m, 1H), 7.16 (t, J=8.8 Hz, 1H), 4.38 (s, 2H), 4.34 (br d, J=8.0 Hz, 1H), 3.95 (s, 2H), 3.75 (s, 4H), 3.71-3.47 (m, 7H), 2.61-2.46 (m, 4H), 2.29 (s, 2H), 2.19 (br t, J=10.4 Hz, 2H), 2.03 (s, 1H), 2.01-1.87 (m, 4H), 1.80-1.54 (m, 9H), 1.43 (s, 9H), 1.28-1.15 (m, 3H), 1.10-0.96 (m, 2H). ¹⁹F NMR (377 MHz, METHANOL-d4) δ=−120.7.

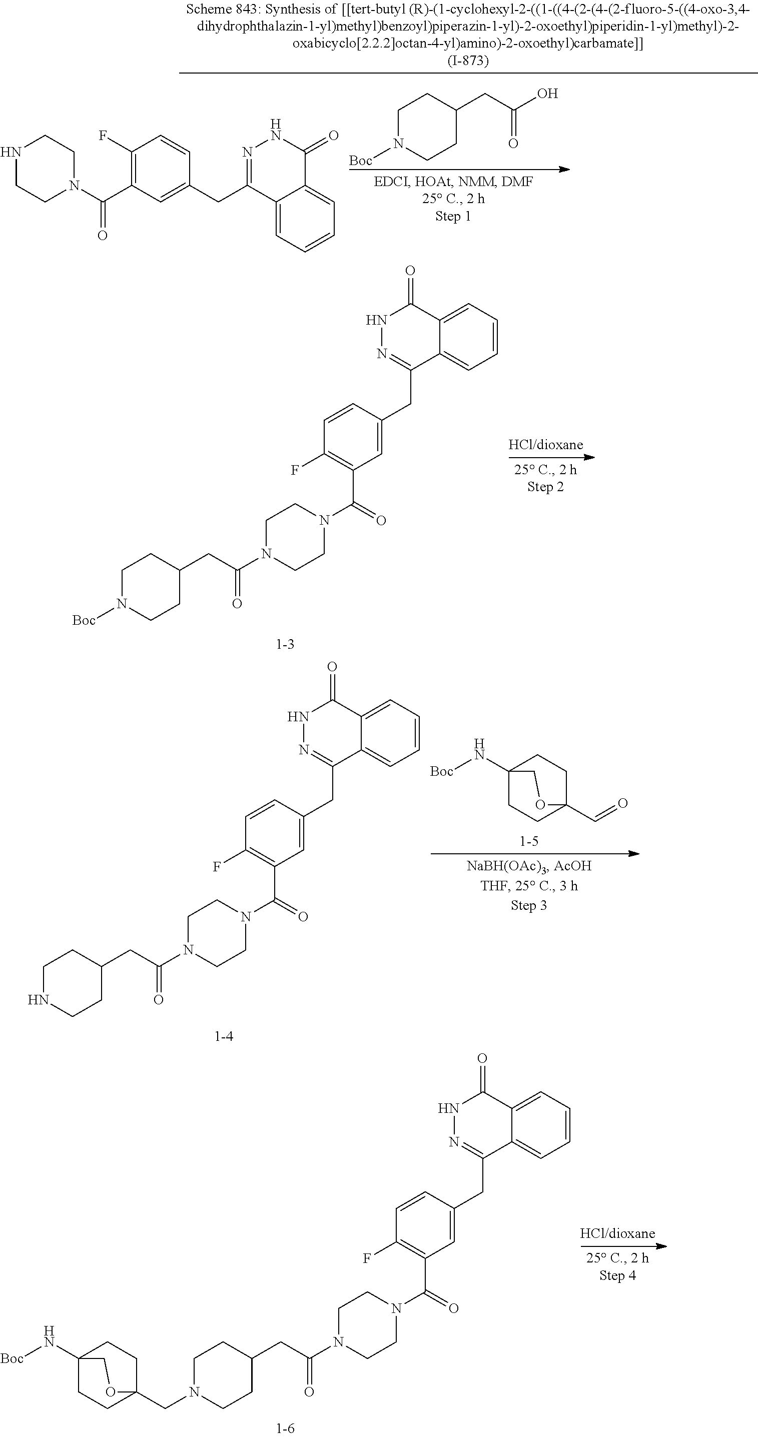

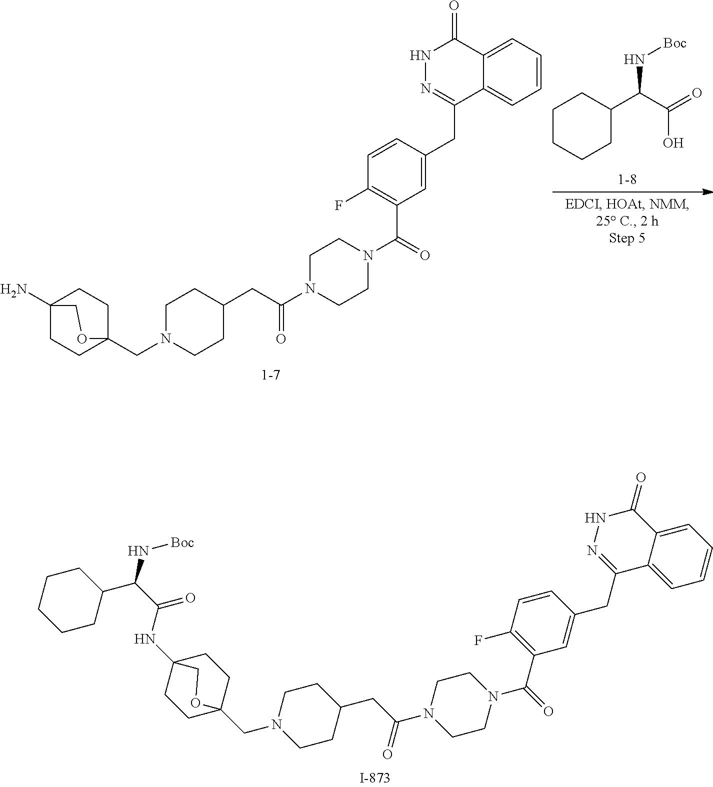

### Step 1: Synthesis of Intermediate 1-3

To a solution of Intermediate 1-2 (219.14 mg, 900.68 μmol, 1.1 eq) in DMF (6 mL) was added HOAt (222.90 mg, 1.64 mmol, 229.08 μL, 2 eq) and EDCI (313.93 mg, 1.64 mmol, 2 eq). The mixture was stirred at 25° C. for 30 min. Then Intermediate 1-1 (300 mg, 818.80 μmol, 1 eq) and NMM (248.46 mg, 2.46 mmol, 270.06 μL, 3 eq) was added and the mixture was stirred at 25° C. for 2 hr. The mixture was poured into 20 mL of water and extracted with EA (20 mL*2). The combined organic phase was dried over anhydrous Na₂SO₄, filtered and concentrated in vacuum. The crude product was purified by reversed-phase HPLC (0.1% FA condition). Intermediate 1-3 (420 mg, 709.85 μmol, 86.69% yield) was obtained as yellow solid. LCMS (Method E): Rt=0.502 min, [M+H]⁺=592.4. ¹H NMR (400 MHz, CHLOROFORM-d) δ=9.92 (br s, 1H), 8.53-8.43 (m, 1H), 7.83-7.68 (m, 3H), 7.40-7.30 (m, 2H), 7.06 (dt, J=3.8, 8.5 Hz, 1H), 4.29 (s, 2H), 4.12-4.03 (m, 2H), 3.85-3.71 (m, 2H), 3.62-3.53 (m, 2H), 3.44 (br d, J=4.4 Hz, 1H), 3.38-3.19 (m, 2H), 2.78-2.65 (m, 2H), 2.33-2.18 (m, 2H), 1.78-1.68 (m, 2H), 1.46 (s, 9H), 1.21-1.07 (m, 2H).

### Step 2: Synthesis of Intermediate 1-4

A mixture of Intermediate 1-3 (420 mg, 709.85 μmol, 1 eq) and HCl/dioxane (2 M, 10 mL, 28.17 eq) was stirred at 25° C. for 2 hr. The mixture was concentrated in vacuum and dissolved in 10 mL of water, basified to pH=10 with aq. Na₂CO₃, and extracted with DCM (10 mL*2). The combined organic phase was dried over anhydrous Na₂SO₄, filtered and concentrated in vacuum. The residue was used directly without further purification. Intermediate 1-4 (340 mg, 691.68 μmol, 97.44% yield) was obtained as a white solid. LCMS (Method E): Rt=0.376 min, [M+H]⁺=492.3. ¹H NMR (400 MHz, DMSO-d6) δ=12.76-12.34 (m, 1H), 8.26 (d, J=7.8 Hz, 1H), 8.01-7.94 (m, 1H), 7.89 (br t, J=7.6 Hz, 1H), 7.86-7.80 (m, 1H), 7.48-7.40 (m, 1H), 7.39-7.33 (m, 1H), 7.24 (br t, J=8.6 Hz, 1H), 4.33 (s, 2H), 3.65-3.47 (m, 13H), 3.19-3.10 (m, 3H), 2.94-2.87 (m, 2H), 2.27-2.15 (m, 3H), 1.82-1.68 (m, 2H), 1.67-1.51 (m, 3H).

### Step 3: Synthesis of Intermediate 1-6

To a solution of Intermediate 1-5 (176.59 mg, 691.68 μmol, 1 eq) and Intermediate 1-4 (340 mg, 691.68 μmol, 1 eq) in DCM (7 mL) was added AcOH (415.37 g, 6.92 μmol, 3.96e−1 μL, 0.01 eq). The mixture was stirred at 25° C. for 1 hr. Then NaBH(OAc)₃ (439.79 mg, 2.08 mmol, 3 eq) was added and the mixture was stirred at 25° C. for 1 hr. The mixture was poured into 20 mL of aq. NH₄Cl and extracted with DCM (20 mL*2). The combined organic phase was dried over anhydrous Na₂SO₄, filtered and concentrated in vacuum. The crude product was purified by reversed-phase chromatography (0.1% FA condition). Intermediate 1-6 (350 mg, 478.88 μmol, 69.23% yield) was obtained as yellow gum. LCMS (Method E): Rt=0.453 min, [M+H]⁺=731.4. ¹H NMR (400 MHz, CHLOROFORM-d) δ=10.10-9.96 (m, 1H), 8.52-8.40 (m, 1H), 7.81-7.67 (m, 3H), 7.39-7.29 (m, 2H), 7.06 (br t, J=8.4 Hz, 1H), 4.31-4.26 (m, 3H), 3.97 (s, 1H), 3.90 (s, 2H), 3.83-3.71 (m, 2H), 3.57 (br d, J=4.9 Hz, 2H), 3.45 (br s, 1H), 3.40 (d, J=6.0 Hz, 1H), 3.36-3.18 (m, 2H), 2.96-2.82 (m, 2H), 2.30-2.17 (m, 4H), 2.15-1.91 (m, 19H), 1.89-1.75 (m, 5H), 1.69-1.62 (m, 4H), 1.43 (d, J=3.0 Hz, 14H), 1.33-1.23 (m, 3H). ¹⁹F NMR (377 MHz, METHANOL-d4) δ=−114.92-−119.66 (m, 1F).

### Step 4: Synthesis of Intermediate 1-7

A mixture of Intermediate 1-6 (350 mg, 478.88 μmol, 1 eq) and HCl/dioxane (2 M, 10 mL, 41.76 eq) was stirred at 25° C. for 2 hr. The mixture was concentrated in vacuum. The residue was used directly without further purification. Intermediate 1-7 (320 mg, crude, HCl salt) was obtained as white solid. LCMS (Method E): Rt=0.406 min, [M+H]⁺=631.3 H NMR (400 MHz, CHLOROFORM-d) δ=12.64 (s, 1H), 9.34-9.09 (m, 1H), 9.01-8.83 (m, 1H), 8.51-8.39 (m, 2H), 8.31 (br d, J=7.5 Hz, 1H), 8.25-8.17 (m, 1H), 8.05-7.97 (m, 1H), 7.96-7.85 (m, 2H), 7.50 (br s, 1H), 7.44-7.34 (m, 1H), 7.33-7.22 (m, 1H), 6.85-6.57 (m, 1H), 4.38 (s, 2H), 3.85 (br s, 2H), 3.80-3.73 (m, 2H), 3.71-3.55 (m, 26H), 1.94 (br d, J=6.0 Hz, 4H), 1.89-1.79 (m, 6H). ¹⁹F NMR (377 MHz, METHANOL-d4) δ=−118.82-−121.05 (m, 1F).

### Step 5: Synthesis of I-873

To a solution of Intermediate 1-8 (185.12 mg, 719.41 μmol, 1.5 eq) in DMF (6 mL) was added HOAt (195.84 mg, 1.44 mmol, 201.27 μL, 3 eq) and EDCI (275.82 mg, 1.44 mmol, 3 eq). The mixture was stirred at 25° C. for 30 min. Then Intermediate 1-7 (320 mg, 479.61 μmol, 1 eq, HCl salt) and NMM (242.55 mg, 2.40 mmol, 263.65 μL, 5 eq) was added and the mixture was stirred at 25° C. for 2 hr. The mixture was poured into 10 mL of water and extracted with EA (10 mL*2). The combined organic phase was dried over anhydrous Na₂SO₄, filtered and concentrated in vacuum. The crude product was purified by reversed-phase chromatography (0.1% FA condition). I-873 (85.7 mg, 93.48 μmol, 19.49% yield, 99.926% purity, FA) was obtained as yellow solid. LCMS (Method E): Rt=0.477 min, [M+H]⁺=870.5. SCF: Rt=1.757 min. ¹H NMR (400 MHz, CHLOROFORM-d) δ=10.60-10.41 (m, 1H), 10.30-10.14 (m, 1H), 8.50-8.45 (m, 1H), 8.45 (s, 1H), 7.88-7.65 (m, 3H), 7.39-7.29 (m, 2H), 7.06 (t, J=8.5 Hz, 1H), 5.71-5.48 (m, 1H), 5.13-4.86 (m, 1H), 4.29 (s, 2H), 4.05-3.95 (m, 2H), 3.85-3.66 (m, 4H), 3.62-3.52 (m, 3H), 3.49-3.24 (m, 6H), 2.71-2.52 (m, 2H), 2.39 (q, J=10.8 Hz, 2H), 2.33-2.22 (m, 2H), 2.22-2.10 (m, 2H), 2.09-1.93 (m, 3H), 1.88-1.73 (m, 7H), 1.67-1.55 (m, 4H), 1.44 (s, 9H), 1.31-1.17 (m, 2H), 1.17-1.08 (m, 1H), 1.06-0.89 (m, 2H). ¹⁹F NMR (377 MHz, METHANOL-d4) δ=−117.58 (br d, J=26.9 Hz, 1F).

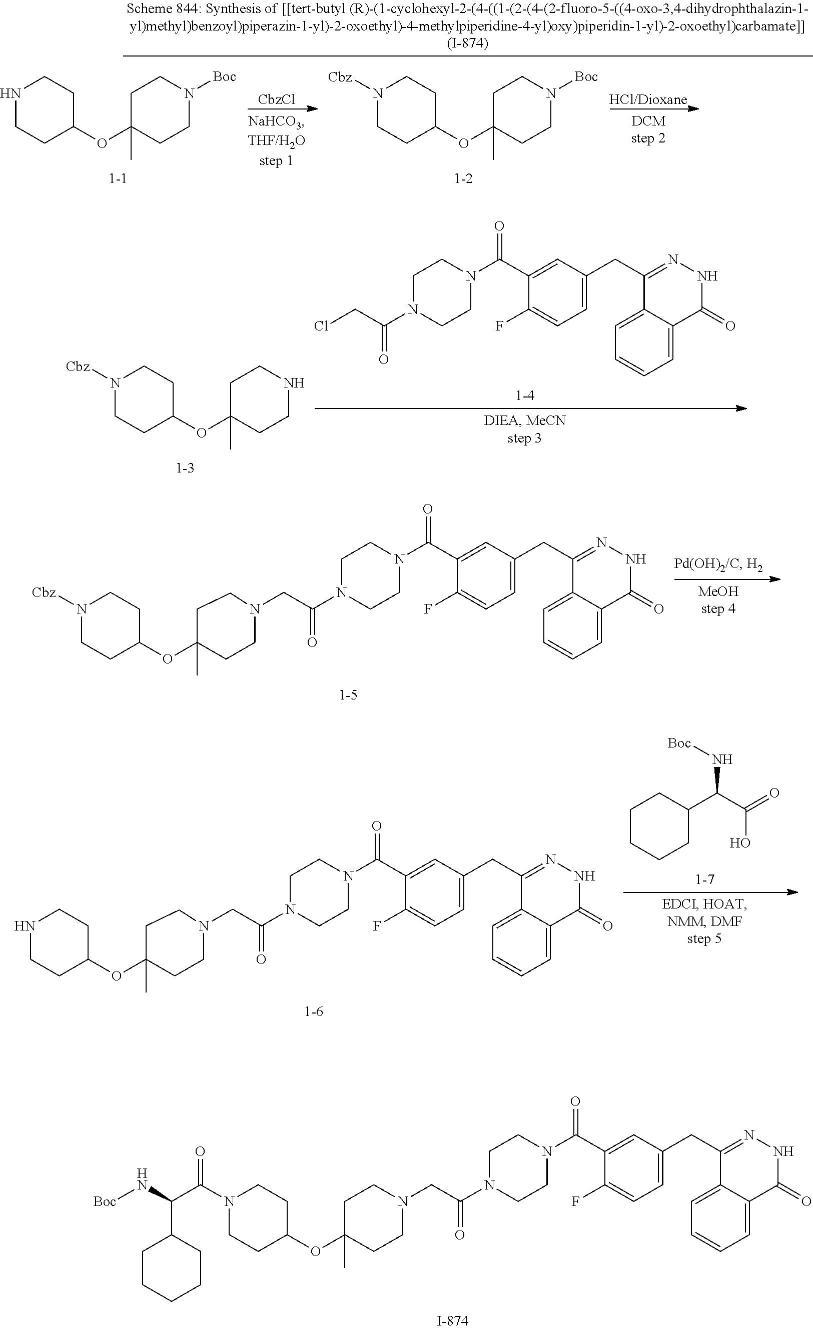

### Step 1: Synthesis of Intermediate 1-2

To a solution of Intermediate 1-1 (1.5 g, 5.03 mmol, 1 eq) and NaHCO₃ (1.27 g, 15.08 mmol, 586.74 μL, 3 eq) in THF (7.5 mL) and H₂O (7.5 mL) was added CbzCl (1.03 g, 6.03 mmol, 861.05 μL, 1.2 eq) at 0° C. The mixture was stirred at 25° C. for 1 hr. The reaction mixture was diluted by H₂O (20 mL) and extracted with EA (10 mL*3). The combined organic layers were washed with brine (10 mL*3), dried over anhydrous Na₂SO₄, filtered and concentrated under reduced pressure to give a residue. The residue was purified by flash silica gel chromatography (ISCO; 40 g SepaFlash Silica Flash Column, Eluent of 0-30% Ethyl acetate/Petroleumethergradient@100 mL/min) and concentrated under vacuum. Intermediate 1-2 (1.5 g, 3.47 mmol, 68.99% yield) was obtained as a colorless oil. ¹H NMR (400 MHz, CHLOROFORM-d) δ=7.37-7.34 (m, 5H), 5.12 (s, 2H), 3.85-3.76 (m, 2H), 3.67 (td, J=4.0, 8.1 Hz, 3H), 3.27-3.15 (m, 4H), 1.67 (br d, J=13.5 Hz, 4H), 1.54-1.49 (m, 2H), 1.46-1.45 (m, 9H), 1.45-1.38 (m, 2H), 1.18 (s, 3H).

### Step 2: Synthesis of Intermediate 1-3

To a solution of Intermediate 1-2 (1.4 g, 3.24 mmol, 1 eq) in DCM (7 mL) was added HCl/dioxane (4 M, 7.00 mL, 8.65 eq). The mixture was stirred at 25° C. for 1 h. The mixture was concentrated under vacuum to give a crude product. The crude was used for the next step directly without purification. Intermediate 1-3 (1 g, crude, HCl salt) was obtained as a yellow oil.

### Step 3: Synthesis of Intermediate 1-5

To a solution of Intermediate 1-3 (1 g, 2.71 mmol, 1 eq, HCl salt) in DMF (10 mL) was added Intermediate 1-4 (1.20 g, 2.71 mmol, 1 eq), KI (1.80 g, 10.84 mmol, 4 eq) and DIEA (1.40 g, 10.84 mmol, 1.89 mL, 4 eq). The mixture was stirred at 25° C. for 1 h. The mixture was filtered. The filtrate was purified by reversed-phase chromatography (0.1% NH₃·H₂O). Intermediate 1-5 (1.7 g, 2.29 mmol, 84.49% yield, 99.538% purity) was obtained as a light yellow solid. LCMS (Method G): Retention time: 0.646 min, [M+H]⁺=739.5.

### Step 4: Synthesis of Intermediate 1-6

To a solution of Pd(OH)₂/C (0.7 g, 1.89 mmol, 20% purity, 1 eq) in MeOH (15 mL) was added Intermediate 1-5 (1.4 g, 1.89 mmol, 1 eq) in MeOH (15 mL) under N₂ atmosphere. The suspension was degassed and purged with H₂ 3 times. The mixture was stirred under H₂ (20 psi) at 25° C. for 2 hr. The mixture was filtered and the filter cake was dried under vacuum to give Intermediate 1-6 (1.2 g, crude) as a white solid. LCMS (Method E): Retention time: 0.398 min, [M+H]⁺=605.3.

### Step 5: Synthesis of I-874

To a solution of Intermediate 1-6 (1.1 g, 1.82 mmol, 1 eq) in DMF (11 mL) was added Intermediate 1-7 (468.09 mg, 1.82 mmol, 1 eq), HOAt (247.59 mg, 1.82 mmol, 254.46 μL, 1 eq), EDCI (697.43 mg, 3.64 mmol, 2 eq) and NMM (919.95 mg, 9.10 mmol, 999.95 μL, 5 eq). The mixture was stirred at 25° C. for 1 h. The mixture was poured into H₂O (33 mL), filtered and washed with H₂O (10 mL*3). The filter cake was concentrated under vacuum to give a crude product. 200 mg of the crude was purified by prep-HPLC (column: Waters Xbridge 150*25 mm*5 um;mobile phase: [water(NH₄HCO₃)-ACN]; gradient: 45%-75% B over 9 min). I-874 (73.84 mg, 86.31 μmol, 4.74% yield, 98.66% purity) was obtained as a white solid. LCMS (Method G): Retention time=0.695 min, [M+H]⁺=844.6. SCF: Retention time=0.770 min. ¹H NMR (400 MHz, DMSO-d6) δ=12.57 (s, 1H), 8.26 (d, J=7.6 Hz, 1H), 8.00-7.93 (m, 1H), 7.92-7.80 (m, 2H), 7.45-7.34 (m, 2H), 7.24-7.22 (m, 1H), 6.82-6.62 (m, 1H), 4.32 (s, 2H), 4.27-4.15 (m, 1H), 4.03-3.73 (m, 3H), 3.64-3.45 (m, 5H), 3.40-3.34 (m, 1H), 3.20-2.90 (m, 6H), 2.40-2.31 (m, 3H), 1.76-1.43 (m, 13H), 1.35 (br s, 11H), 1.12 (br d, J=11.6 Hz, 6H), 0.99-0.87 (m, 2H). ¹⁹F NMR (377 MHz, DMSO-d6) δ=−119.77 (br d, J=11.2 Hz, 1F).

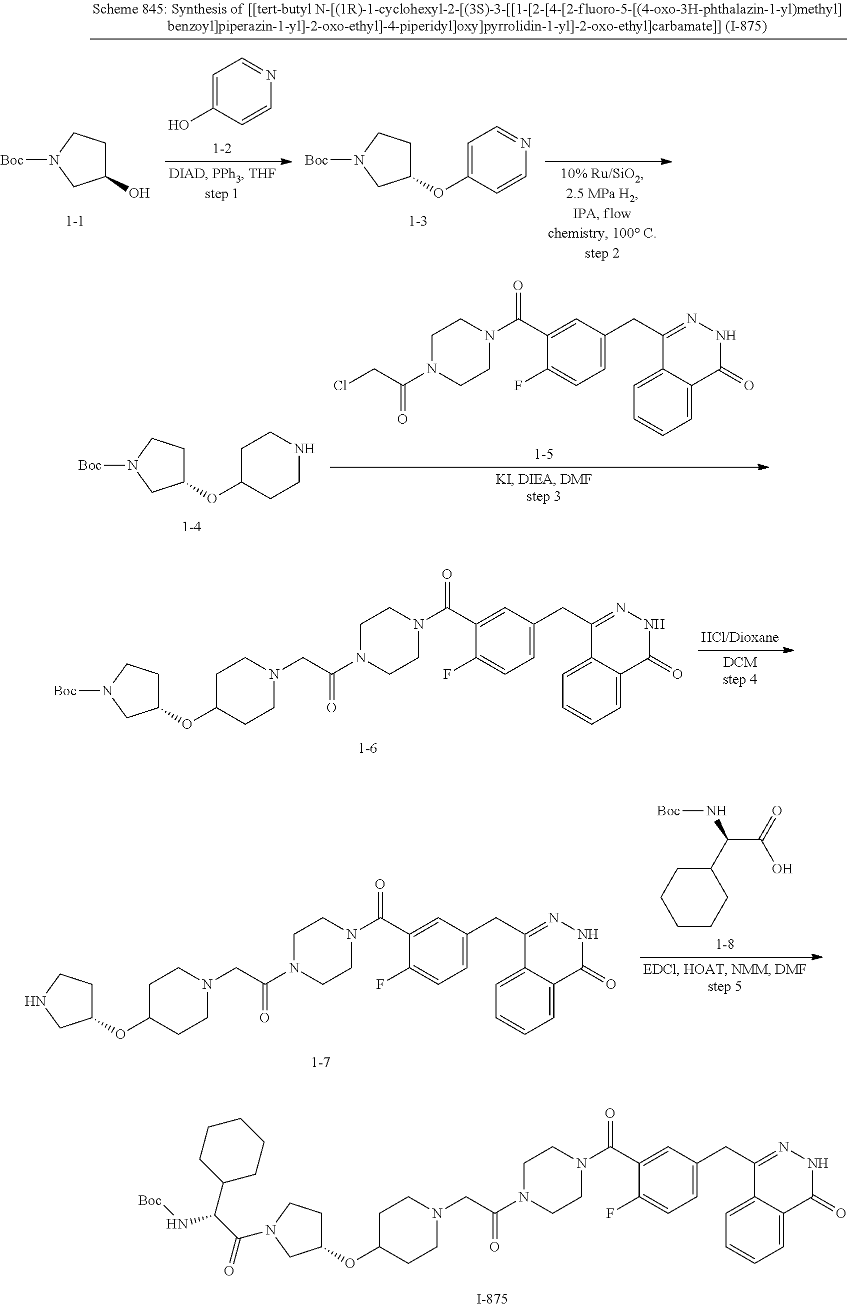

### Step 1: Synthesis of Intermediate 1-3

To a solution of Intermediate 1-1 (5 g, 26.70 mmol, 1 eq), Intermediate 1-2 (3.05 g, 32.05 mmol, 1.2 eq) and DIAD (10.80 g, 53.41 mmol, 10.35 mL, 2 eq) in THF (50 mL) was added PPh₃ (14.01 g, 53.41 mmol, 2 eq) at 0° C. The mixture was stirred at 40° C. for 1 hr. The mixture was diluted with water (10 mL), extracted with EA (15 mL*3) and washed with brine (5 mL*3). The organic layer was dried with anhydrous Na₂SO₄, filtered and concentrated under vacuum. The residue was purified by column chromatography (SiO₂, Petroleum ether/Ethyl acetate=1/0 to 0/1) and concentrated under vacuum. Intermediate 1-3 (4.7 g, 17.78 mmol, 66.59% yield) was obtained as yellow oil. LCMS (Method E): Rt=0.537 min, [M+H]=265.1. SCF: Rt=1.888 min. ¹H NMR (400 MHz, DMSO-d6) δ=8.42-8.35 (m, 2H), 7.00-6.93 (m, 2H), 5.12 (br s, 1H), 3.63-3.54 (m, 1H), 3.47-3.36 (m, 2H), 3.31 (br s, 1H), 2.25-2.09 (m, 1H), 2.08-1.97 (m, 1H), 1.39 (br d, J=5.6 Hz, 9H).

### Step 2: Synthesis of Intermediate 1-4

A solution of Intermediate 1-3 (1 eq, 4 g) in IPA (100 mL) was prepared. A fixed bed (volume 5 mL) was packed with granular catalyst (10% Ru/SiO₂, 2 g). The H₂ back pressure regulator was adjusted to 2.5 MPa and the flow rate of H₂ was set to 20 mL/min. Then the solution was pumped (0.303 mL/min) to the fixed bed (6.350(¼″) mm, 1 mL, 100° C.). The solution was pumped through the fixed bed and then the reaction mixture was collected from the reactor output. After the reaction fluid injection completed, the fixed bed was washed by extra IPA (100 mL). The organic layer was concentrated under vacuum. Intermediate 1-4 (3.6 g, crude) was obtained as a colorless oil. LCMS (Method E): Rt=0.372 min, [M+H]+=271.1. ¹H NMR (400 MHz, CHLOROFORM-d) δ=4.13 (br s, 1H), 3.72 (br s, 2H), 3.42-3.21 (m, 4H), 3.16-3.05 (m, 2H), 2.78-2.67 (m, 2H), 1.90 (br s, 4H), 1.60-1.47 (m, 2H), 1.43 (s, 9H).

### Step 3: Synthesis of Intermediate 1-6

To a solution of Intermediate 1-4 (500 mg, 1.85 mmol, 1 eq) in DMF (5 mL) was added Intermediate 1-5 (819.02 mg, 1.85 mmol, 1 eq), DIEA (956.05 mg, 7.40 mmol, 1.29 mL, 4 eq) and KI (1.23 g, 7.40 mmol, 4 eq). The mixture was stirred at 25° C. for 1 hr. The mixture was diluted with water (2 mL), extracted with EtOAc (10 mL*3) and washed with brine (5 mL*4). The organic layer was dried with anhydrous Na₂SO₄, filtered and concentrated under vacuum. The residue was purified by column chromatography (SiO₂, Dichloromethane: Methanol=1/0 to 10/1) and the mixture was concentrated under vacuum to give Intermediate 1-6 (700 mg, 1.03 mmol, 55.93% yield) as a white solid. LCMS (Method G): Rt=0.589 min, [M+H]⁺=677.3. SCF: Rt=2.048 min. ¹H NMR (400 MHz, DMSO-d6) δ=12.59 (s, 1H), 8.26 (d, J=7.6 Hz, 1H), 8.00-7.94 (m, 1H), 7.88 (t, J=7.2 Hz, 1H), 7.85-7.79 (m, 1H), 7.43 (br s, 1H), 7.38 (br t, J=5.1 Hz, 1H), 7.23 (t, J=9.0 Hz, 1H), 4.33 (s, 2H), 4.14 (br s, 1H), 3.67-3.54 (m, 3H), 3.48 (br d, J=14.5 Hz, 2H), 3.39-3.29 (m, 5H), 3.24 (br s, 2H), 3.15 (br d, J=9.0 Hz, 4H), 3.07 (br s, 1H), 2.65 (br s, 2H), 2.17-2.04 (m, 2H), 1.79 (br d, J=12.6 Hz, 4H), 1.38 (br s, 9H). ¹⁹F NMR (377 MHz, DMSO-d6) δ=−73.41 (s, 1F), −119.76 (br s, 1F).

### Step 4: Synthesis of Intermediate 1-7

To a solution of Intermediate 1-6 (100 mg, 147.76 μmol, 1 eq) in DCM (0.3 mL) was added HCl/dioxane (2 M, 0.6 mL, 8.12 eq). The mixture was stirred at 25° C. for 1 hr. The mixture was concentrated under vacuum. The residue was used in the next step without purification. Intermediate 1-7 (90 mg, crude, HCl salt) was obtained as white solid. LCMS (Method F): Rt=0.651 min, [M+H]⁺=577.4.

### Step 5: Synthesis of I-875

To a solution of Intermediate 1-7 (990 mg, 1.61 mmol, 1 eq, HCl salt) in DMF (10 mL) was added Intermediate 1-8 (415.50 mg, 1.61 mmol, 1 eq), EDCI (619.08 mg, 3.23 mmol, 2 eq), HOAt (219.78 mg, 1.61 mmol, 225.88 μL, 1 eq) and NMM (816.60 mg, 8.07 mmol, 887.61 μL, 5 eq). The mixture was stirred at 25° C. for 1 hr. The mixture was diluted with water (10 mL), and extracted with EtOAc (20 mL*3). The mixture was diluted with water (10 mL), and extracted with EtOAc (20 mL*3) and washed with brine (5 mL*3). The organic layer was dried with anhydrous Na₂SO₄, filtered and concentrated under vacuum. The crude product was triturated with PE/EA=20/1 (50 mL), filtered and the filter cake was washed with PE/EA=20/1 (5 mL*3), the filter cake was dried under vacuum and was obtained as a product. A part of the product was purified by prep-HPLC (column: Waters Xbridge 150*25 mm*5 um; mobile phase: [water (NH₄HCO₃)-ACN]; gradient: 30%-60% B over 15 min) and dried by lyophilization, then purified by Prep-HPLC (column: Phenomenex luna C18 150*25 mm*10 um; mobile phase: [water (FA)-ACN]; gradient: 18%-48% B over 15 min) and dried by lyophilization. I-875 (1000 mg, 75.08% yield, 99.92% purity, FA salt) was obtained as a white solid. LCMS (Method G): Rt=0.630 min, [M+H]⁺=816.5. SCF: Rt=1.441 min. ¹H NMR (400 MHz, DMSO-d6) δ=12.61 (s, 1H), 8.27 (d, J=7.6 Hz, 1H), 8.21 (s, 1H), 8.01-7.95 (m, 1H), 7.87-7.92 (m, 1H), 7.87-7.81 (m, 1H), 7.45 (br d, J=0.8 Hz, 1H), 7.35-7.41 (m, 1H), 7.21-7.27 (m, 1H), 6.95-6.43 (m, 1H), 4.34 (s, 2H), 4.26-4.14 (m, 1H), 4.03-3.83 (m, 1H), 3.73-3.37 (m, 10H), 3.30-3.07 (m, 5H), 2.70-2.58 (m, 2H), 2.19-2.06 (m, 2H), 1.95 (br s, 1H), 1.89-1.70 (m, 4H), 1.70-1.54 (m, 4H), 1.53-1.41 (m, 2H), 1.38-1.33 (m, 9H), 1.14-1.04 (m, 3H), 0.93 (br d, J=10.0 Hz, 2H). ¹⁹F NMR (377 MHz, DMSO-d6) δ=−119.80 (br s, 1F).

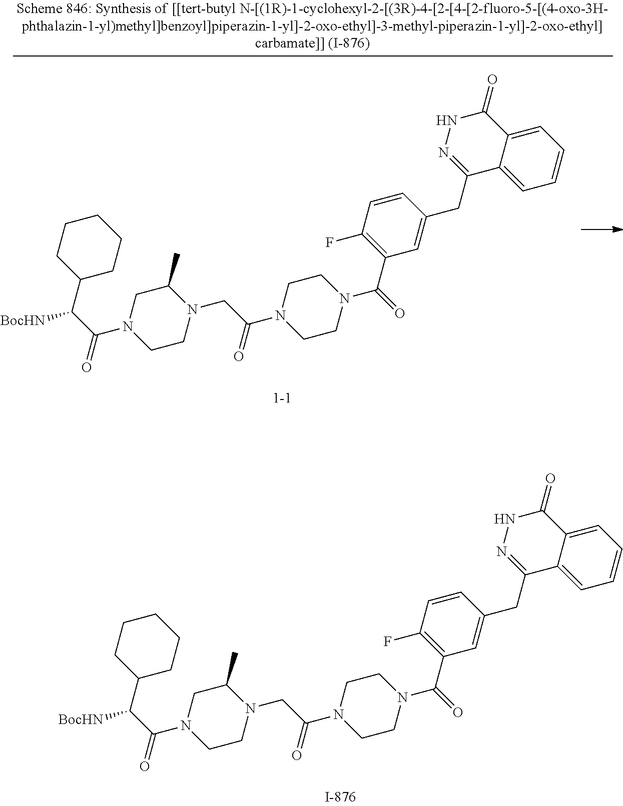 [SMILES: C[C@@H]1CN(C(=O)[C@H](NC(=O)OC(C)(C)C)C2CCCCC2)CCN1CC(=O)N1CCN(C(=O)c2cc(Cc3n[nH]c(=O)c4ccccc34)ccc2F)CC1 ; C[C@@H]1CN(C(=O)[C@H](NC(=O)OC(C)(C)C)C2CCCCC2)CCN1CC(=O)N1CCN(C(=O)c2cc(Cc3n[nH]c(=O)c4ccccc34)ccc2F)CC1]

The Intermediate 1-1 was repurified by prep-HPLC (column: CD02-Waters Xbidge BEH C18 150*25*10 um; mobile phase: [water(FA)-ACN]; gradient: 18%-48% B over 10 min) and the eluent was concentrated under reduced pressure to remove ACN and lyophilized to give product. I-876 (152.1 mg, 201.23 μmol, 75.05% yield, 98.68% purity) was obtained as a white solid. LCMS (Method D): Rt=0.385 min, [M+H]⁺=746.3. SCF: Rt=4.759 min, 6.412 min. ¹H NMR (400 MHz, METHANOL-d4) δ=8.40-8.33 (m, 1H), 7.98-7.93 (m, 1H), 7.90-7.80 (m, 2H), 7.53-7.46 (m, 1H), 7.38-7.37 (m, 1H), 7.16-7.14 (m, 1H), 6.57-6.55 (m, 1H), 4.39 (s, 2H), 4.36 (s, 1H), 4.03-3.90 (m, 1H), 3.88-3.80 (m, 2H), 3.79-3.63 (m, 4H), 3.62-3.50 (m, 2H), 3.49-3.41 (m, 1H), 3.36 (s, 1H), 3.30-3.23 (m, 1H), 3.21-2.93 (m, 2H), 2.86-2.75 (m, 1H), 2.68-2.48 (m, 1H), 2.46-2.29 (m, 1H), 1.76 (d, J=7.4 Hz, 3H), 1.69-1.58 (m, 3H), 1.43 (s, 9H), 1.30-1.18 (m, 3H), 1.12 (d, J=6.4 Hz, 1H), 1.10-1.06 (m, 2H), 1.04 (d, J=6.0 Hz, 2H). ¹⁹F NMR (376 MHz, METHANOL-d4) δ=−120.711.

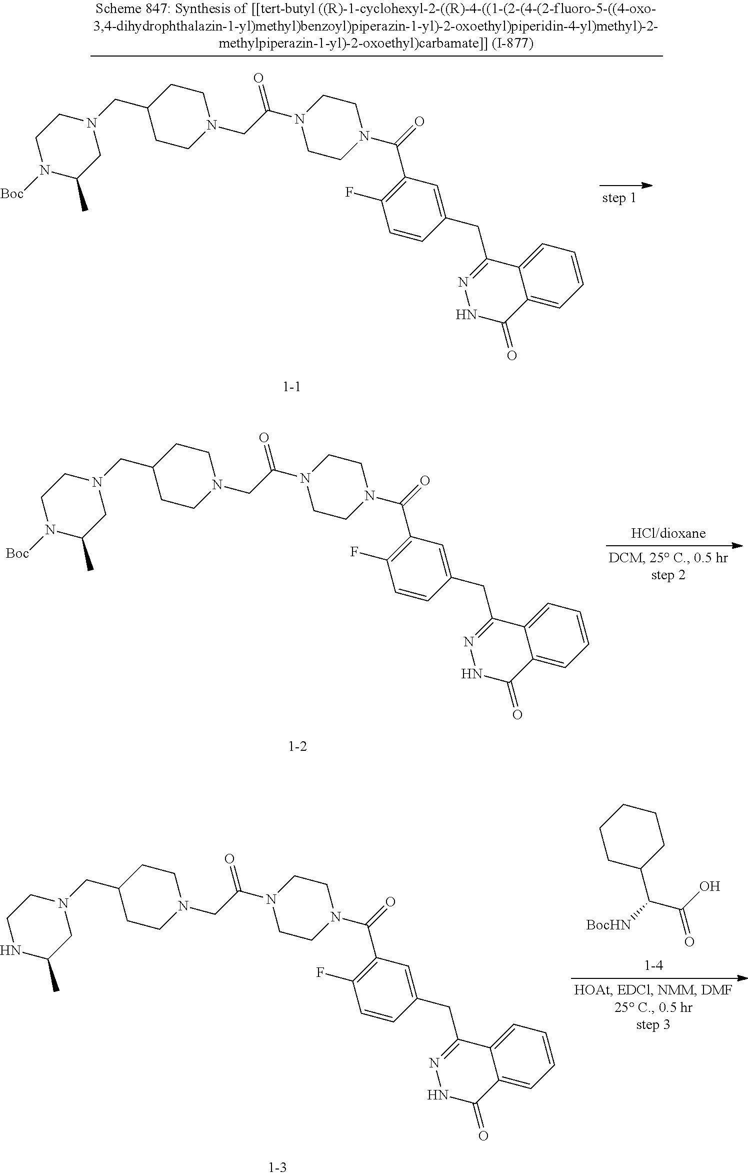 [SMILES: C[C@@H]1CN(CC2CCN(CC(=O)N3CCN(C(=O)c4cc(Cc5n[nH]c(=O)c6ccccc56)ccc4F)CC3)CC2)CCN1C(=O)OC(C)(C)C ; C[C@@H]1CN(CC2CCN(CC(=O)N3CCN(C(=O)c4cc(Cc5n[nH]c(=O)c6ccccc56)ccc4F)CC3)CC2)CCN1C(=O)OC(C)(C)C ; C[C@@H]1CN(CC2CCN(CC(=O)N3CCN(C(=O)c4cc(Cc5n[nH]c(=O)c6ccccc56)ccc4F)CC3)CC2)CCN1 ; CC(C)(C)OC(=O)N[C@@H](C(=O)O)C1CCCCC1]

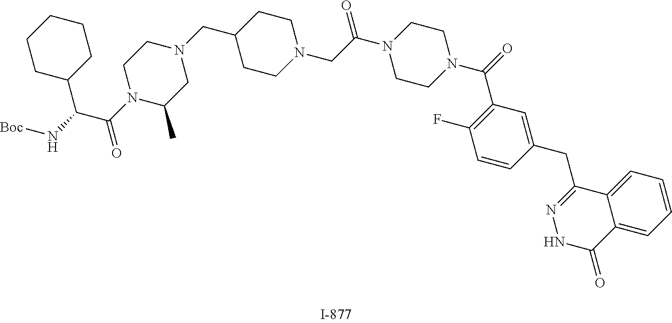 [SMILES: C[C@@H]1CN(CC2CCN(CC(=O)N3CCN(C(=O)c4cc(Cc5n[nH]c(=O)c6ccccc56)ccc4F)CC3)CC2)CCN1C(=O)[C@H](NC(=O)OC(C)(C)C)C1CCCCC1]

### Step 1: Synthesis of Intermediate 1-2.

Intermediate 1-1 was re-purified by prep-HPLC (column: CD06-Waters Xbidge C18 150*40*10 um; mobile phase: [water(NH4HCO3)-ACN]; gradient: 36%-66% B over 10 min), the eluent was concentrated and lyophilized to give the desired product. Intermediate 1-2 (500 mg, 697.09 μmol, 81.77% yield, 98.129% purity) was obtained as a yellow solid. LCMS (Method D): Retention time: 0.311 min, [M+H]⁺=704.4. SCF: Retention time: 1.246 min. ¹H NMR (400 MHz, METHANOL-d4) δ=8.37 (d, J=7.6 Hz, 1H), 7.98-7.92 (m, 1H), 7.91-7.79 (m, 2H), 7.48 (d, J=2.4 Hz, 1H), 7.42-7.34 (m, 1H), 7.19-7.11 (m, 1H), 4.38 (s, 2H), 4.16 (s, 1H), 3.82-3.71 (m, 4H), 3.66 (s, 1H), 3.60-3.49 (m, 2H), 3.33 (d, J=0.8 Hz, 1H), 3.25-3.17 (m, 2H), 3.12-3.02 (m, 1H), 2.96-2.84 (m, 2H), 2.79-2.64 (m, 2H), 2.19-2.00 (m, 6H), 1.94-1.85 (m, 1H), 1.76 (d, J=10.8 Hz, 2H), 1.50 (d, J=4.4 Hz, 1H), 1.45 (s, 9H), 1.28-1.16 (m, 5H). 19F NMR (376 MHz, METHANOL-d4) δ=−120.696.

### Step 2: Synthesis of Intermediate 1-3.

To a solution of Intermediate 1-2 (400 mg, 568.31 μmol, 1 eq) in DCM (10 mL) was added HCl/dioxane (2 M, 10 mL). The mixture was stirred at 25° C. for 0.5 hr. The reaction mixture was concentrated under reduced pressure to give a residue. The crude product was used in the next step without further purification. Intermediate 1-3 (360 mg, crude, HCl salt) was obtained as a white solid. LCMS (Method D): Retention time: 0.195 min, [M+H]⁺=604.3.

### Step 3: Synthesis of I-877

To a solution of Intermediate 1-3 (300 mg, 468.61 μmol, 1 eq, HCl salt) and Intermediate 1-4 (120.59 mg, 468.61 μmol, 1 eq) in DMF (3 mL) was added HOAt (63.78 mg, 468.61 μmol, 65.55 μL, 1 eq), EDCI (269.50 mg, 1.41 mmol, 3 eq) and NMM (236.99 mg, 2.34 mmol, 257.60 μL, 5 eq). The mixture was stirred at 25° C. for 0.5 hr. The reaction mixture was diluted with H₂O (5 mL) and extracted with EA (5 mL*3). The combined organic layers were washed with brine (5 mL*3), dried over Na₂SO₄, filtered and concentrated under reduced pressure to give a residue. The crude product was purified by reversed-phase chromatography (neutral condition), and the eluent was concentrated and lyophilized to give the desired product. I-877 (90.91 mg, 106.51 μmol, 22.73% yield, 98.77% purity) was obtained as a white solid. LCMS (Method H): Retention time: 0.726 min, [M+H]⁺=843.5. SCF: Retention time: 0.956 min. ¹H NMR (400 MHz, METHANOL-d4) δ=8.37 (d, J=7.2 Hz, 1H), 7.99-7.92 (m, 1H), 7.91-7.78 (m, 2H), 7.48 (s, 1H), 7.40-7.35 (m, 1H), 7.20-7.11 (m, 1H), 4.62-4.45 (m, 1H), 4.38 (s, 2H), 4.34-4.25 (m, 1H), 3.88-3.63 (m, 5H), 3.61-3.37 (m, 3H), 3.29-3.11 (m, 3H), 3.00-2.83 (m, 3H), 2.77-2.70 (m, 1H), 2.34-1.85 (m, 7H), 1.83-1.49 (m, 10H), 1.43 (s, 9H), 1.30-1.15 (m, 7H), 1.11-0.93 (m, 2H). ¹⁹F NMR (376 MHz, METHANOL-d4) δ=−120.802.

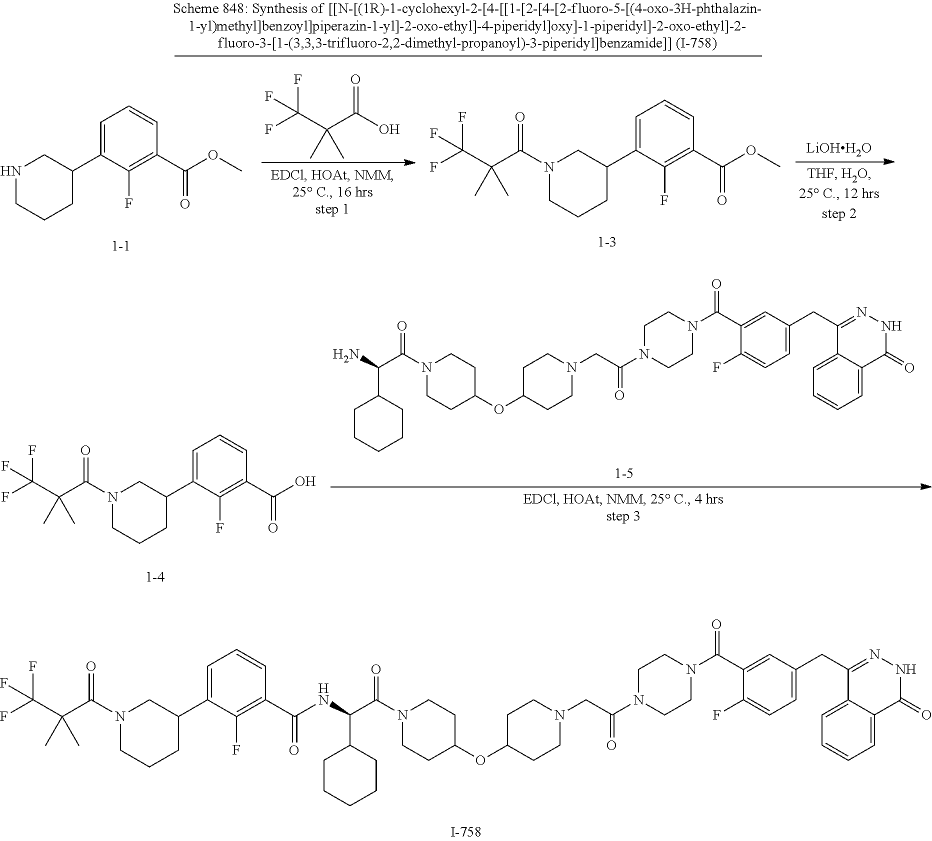

### Step 1: Synthesis of Intermediate 3

To a solution of Intermediate 2 (0.5 g, 3.20 mmol, 1 eq) in DMF (5 mL) was added HOAt (217.98 mg, 1.60 mmol, 224.03 μL, 0.5 eq), EDCI (1.23 g, 6.41 mmol, 2 eq) and NMM (1.62 g, 16.02 mmol, 1.76 mL, 5 eq). Intermediate 1 (1.05 g, 3.84 mmol, 1.2 eq, HCl salt) was added to the mixture and it was stirred at 25° C. for 16 hrs under N₂ atmosphere. The reaction mixture was diluted with H₂O (10 mL) and extracted with EA (15 mL*3). The combined organic layers were washed with brine (10 mL*3), dried over Na₂SO₄, filtered and concentrated under reduced pressure to give a residue. The residue was purified by flash silica gel chromatography (ISCO; 40 g SepaFlash Silica Flash Column, Eluent of 35˜45% Ethyl acetate/Petroleum ether gradient, 50 mL/min) to give Intermediate 3 (1.1 g, 2.93 mmol, 91.49% yield) as a white solid. LCMS (Method E): Rt=0.556 min, [M+H]⁺=376.1. ¹H NMR (400 MHz, CHLOROFORM-d) δ=7.85-7.80 (m, 1H), 7.44-7.39 (m, 1H), 7.21-7.17 (m, 1H), 4.62-4.38 (m, 2H), 3.94 (s, 3H), 3.13-3.02 (m, 1H), 3.01-2.91 (m, 1H), 2.88-2.77 (m, 1H), 2.11-2.00 (m, 1H), 1.93-1.76 (m, 2H), 1.72-1.61 (m, 1H), 1.58-1.55 (m, 6H).

### Step 2: Synthesis of Intermediate 4

To a solution of Intermediate 3 (1 g, 2.66 mmol, 1 eq) in THF (10 mL) and H₂O (2.5 mL) was added LiOH H₂O (335.39 mg, 7.99 mmol, 3 eq). The mixture was stirred at 25° C. for 12 hr under N₂ atmosphere. The reaction mixture was concentrated under reduced pressure to give a residue. The residue was diluted with H₂O (15 mL), adjusted to pH-4 with 1N HCl and extracted with EA (20 mL*3). The combined organic layers were dried over Na₂SO₄, filtered and concentrated under reduced pressure to give a crude product. The crude product was used in the next step without further purification. Intermediate 4 (1 g, crude) was obtained as a white solid. LCMS (Method E): Rt=0.498 min, [M+H]⁺=362.1. ¹H NMR (400 MHz, DMSO-d6) δ=13.22 (br s, 1H), 7.76-7.70 (m, 1H), 7.64-7.60 (m, 1H), 7.30-7.25 (m, 1H), 4.38-4.19 (m, 2H), 3.04-2.84 (m, 3H), 1.95-1.73 (m, 3H), 1.59-1.50 (m, 1H), 1.48 (d, J=6.0 Hz, 6H).

### Step 3: Synthesis of I-758

To a solution of Intermediate 4 (297.03 mg, 822.05 μmol, 1.2 eq) in DMF (5 mL) was added HOAt (46.62 mg, 342.52 μmol, 47.91 μL, 0.5 eq), EDCI (262.65 mg, 1.37 mmol, 2 eq) and NMM (346.45 mg, 3.43 mmol, 376.58 μL, 5 eq) was added Intermediate 5 (0.5 g, 685.04 μmol, 1 eq). The mixture was stirred at 25° C. for 4 hr under N₂ atmosphere. The reaction mixture was diluted with H₂O (10 mL) and extracted with EA (10 mL*3). The combined organic layers were washed with brine (10 mL*3), dried over Na₂SO₄, filtered and concentrated under reduced pressure to give a residue. The residue was purified by reversed phase preparative HPLC (105 g of SepaFlash Spherical C18, 20-45 μm, 100 Å; mobile phase: A for H₂O+0.1% FA and B for acetonitrile; gradient: B 30-50% in 20 min, flow rate: 60 mL/min; column temperature: RT, wavelength: 220 nm/254 nm), and the eluent was concentrated to remove organic solvents and lyophilized. I-758 (352.87 mg, 328.64 μmol, 47.97% yield, 99.95% purity, FA salt) was obtained as a white solid. LCMS (Method E): Rt=0.530 min, [M/2+H]⁺=537.6. ¹H NMR (400 MHz, DMSO-d6) δ=12.69-12.51 (m, 1H), 8.47-8.34 (m, 1H), 8.26 (d, J=7.6 Hz, 1H), 8.15 (s, 1H), 8.00-7.94 (m, 1H), 7.93-7.78 (m, 2H), 7.52-7.33 (m, 4H), 7.28-7.18 (m, 2H), 4.84-4.71 (m, 1H), 4.41-4.23 (m, 4H), 4.03-3.79 (m, 2H), 3.71-3.41 (m, 8H), 3.21-3.07 (m, 5H), 3.06-2.83 (m, 4H), 2.72-2.61 (m, 2H), 2.20-2.06 (m, 2H), 1.94-1.63 (m, 12H), 1.63-1.56 (m, 2H), 1.51-1.46 (m, 6H), 1.45-1.33 (m, 3H), 1.29-0.97 (m, 6H).

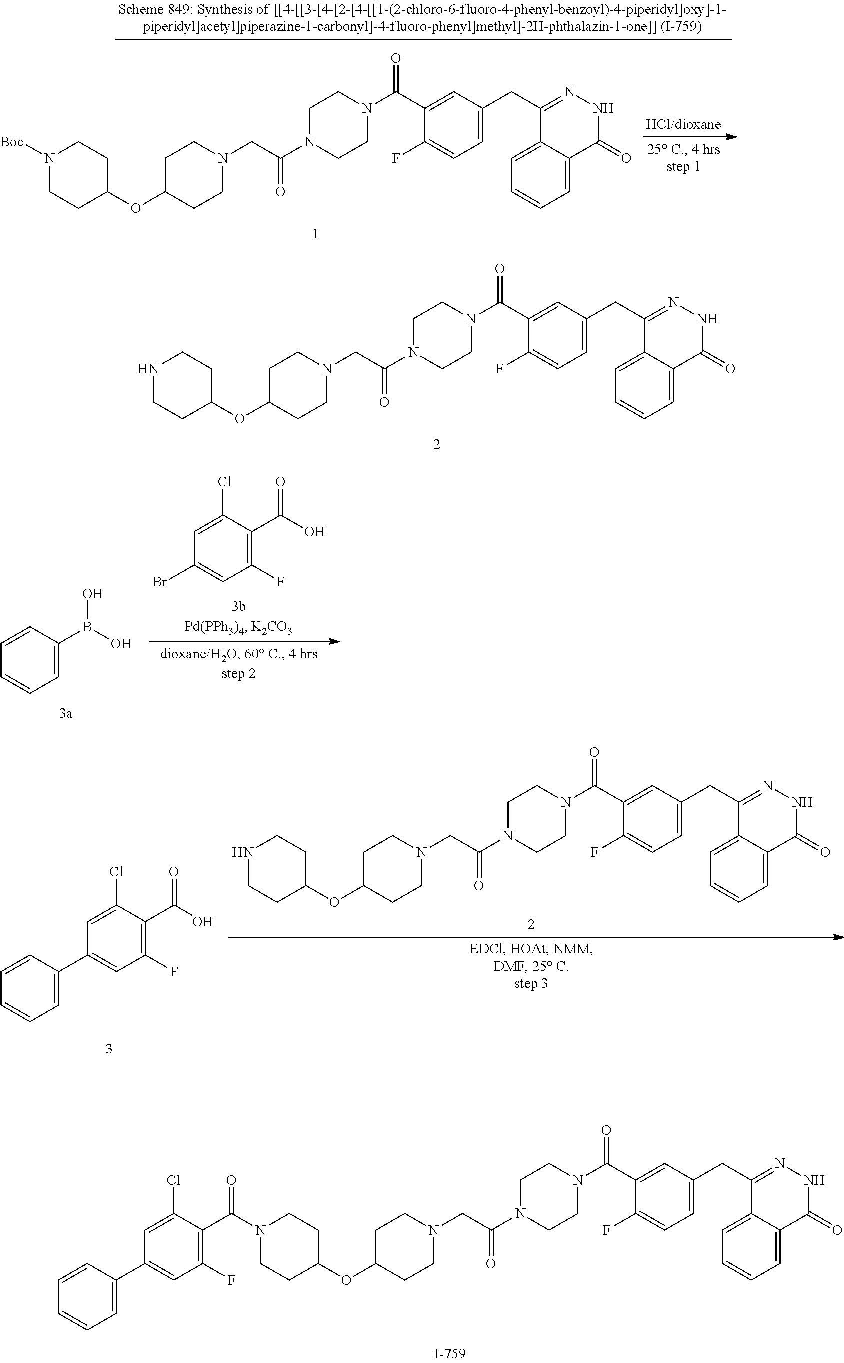

### Step 1: Synthesis of Intermediate 2

To a solution of Intermediate 1 (5 g, 7.24 mmol, 1 eq) in dioxane (5 mL) was added HCl/dioxane (2 M, 50 mL, 13.82 eq). The mixture was stirred at 25° C. for 4 hr under N₂ atmosphere. The reaction mixture was diluted with EA (10 mL), filtered and washed with EA (15 mL). Then the filter cake was collected and dried under reduced pressure. The crude product was used in the next step without further purification. Intermediate 2 (7 g, crude, HCl salt) was obtained as a yellow solid. LCMS (Method E): Rt=0.437 min, [M+H]⁺=591.3. ¹H NMR (400 MHz, DMSO-d6) δ=12.61 (d, J=4.0 Hz, 1H), 9.83-9.55 (m, 1H), 9.24-8.81 (m, 2H), 8.26 (br d, J=8.0 Hz, 1H), 8.00-7.80 (m, 3H), 7.52-7.43 (m, 1H), 7.40-7.32 (m, 1H), 7.29-7.21 (m, 1H), 4.43-4.25 (m, 4H), 3.86-3.60 (m, 7H), 3.29 (br d, J=10.0 Hz, 3H), 3.25-2.99 (m, 6H), 2.99-2.88 (m, 2H), 1.97-1.83 (m, 4H), 1.75-1.58 (m, 4H).

### Step 2: Synthesis of Intermediate 3

To a solution of Intermediate 3a (5 g, 19.73 mmol, 1 eq) in dioxane (50 mL) and H₂O (5 mL) was added Pd(PPh₃)₄ (1.14 g, 986.38 μmol, 0.05 eq), K₂CO₃ (5.45 g, 39.46 mmol, 2 eq) and Intermediate 3b (2.89 g, 23.67 mmol, 1.2 eq). The mixture was stirred at 60° C. for 4 hr under N₂ atmosphere. The reaction mixture was concentrated under reduced pressure to give a residue. The residue was diluted with H₂O (150 mL) and extracted with DCM (100 mL*2). The aqueous phase was acidified with 1N HCl to pH-3. The resulting precipitate was collected by filtration. The crude product was used in the next step without further purification. Intermediate 3 (3.4 g, 13.56 mmol, 68.76% yield) was obtained as a gray solid. H NMR (400 MHz, DMSO-d6) δ=15.60-12.32 (m, 1H), 7.82-7.66 (m, 4H), 7.54-7.42 (m, 3H).

### Step 3: Synthesis of I-759

To a solution of Intermediate 3 (239.80 mg, 956.71 μmol, 1.2 eq) in DMF (5 mL) was added HOAt (54.26 mg, 398.63 μmol, 55.76 μL, 0.5 eq), NMM (403.20 mg, 3.99 mmol, 438.26 μL, 5 eq) and EDCI (305.67 mg, 1.59 mmol, 2 eq), was added Intermediate 2 (0.5 g, 797.26 μmol, 1 eq, HCl salt). The mixture was stirred at 25° C. for 12 hr under N₂ atmosphere. The reaction mixture was diluted with H₂O (10 mL) and extracted with EA (10 mL*3). The combined organic layers were washed with brine (10 mL*3), dried over Na₂SO₄, filtered and concentrated under reduced pressure to give a residue. The residue was purified by reversed phase chromatography (105 g of SepaFlash Spherical C18, 20-45 μm, 100 Å; mobile phase: A for H₂O+0.1% NH₃·H₂O and B for acetonitrile; gradient: B 40-60% in 15 min, flow rate: 50 mL/min; column temperature: RT, wavelength: 220 nm/254 nm), the eluent was concentrated to remove organic solvents and lyophilized to give a crude product. The crude product was re-purified by reversed phase chromatography (105 g of SepaFlash Spherical C18, 20-45 μm, 100 Å; mobile phase: A for H₂O+0.10% FA and B for acetonitrile; gradient: B 30-50% in 20 min, flow rate: 60 mL/min; column temperature: RT, wavelength: 220 nm/254 nm), and the eluent was concentrated to remove organic solvents and lyophilized. I-759 (291.73 mg, 354.05 μmol, 44.41% yield, 99.92% purity, FA salt) was obtained as a white solid. LCMS (Method E): Rt=0.491 min, [M+H]⁺=823.5. ¹H NMR (400 MHz, DMSO-d6) δ=12.59 (s, 1H), 8.26 (d, J=7.6 Hz, 1H), 8.16 (s, 1H), 8.00-7.94 (m, 1H), 7.92-7.86 (m, 1H), 7.86-7.73 (m, 4H), 7.72-7.66 (m, 1H), 7.54-7.41 (m, 4H), 7.40-7.33 (m, 1H), 7.27-7.20 (m, 1H), 4.33 (s, 2H), 4.11-3.93 (m, 1H), 3.77-3.54 (m, 4H), 3.53-3.36 (m, 6H), 3.19-3.07 (m, 5H), 2.74-2.59 (m, 2H), 2.20-2.04 (m, 2H), 1.92-1.69 (m, 4H), 1.52-1.28 (m, 4H).

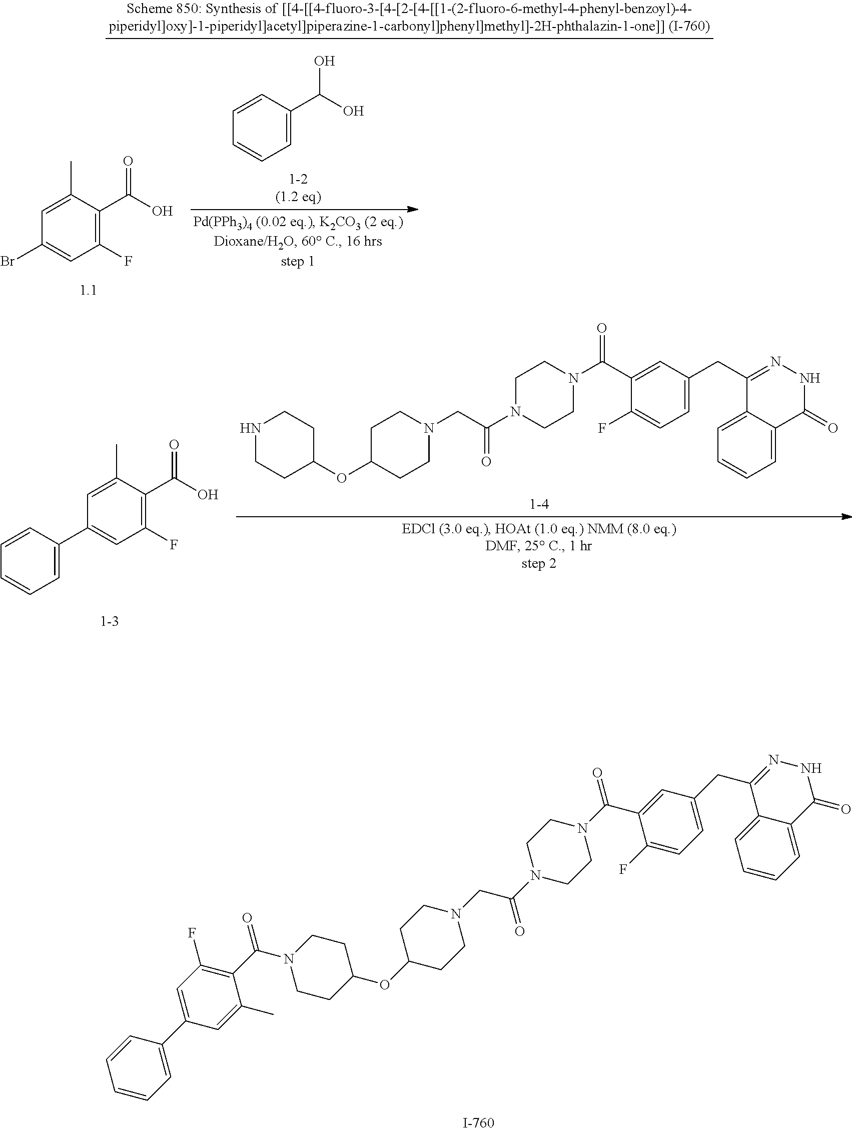 [SMILES: Cc1cc(Br)cc(F)c1C(=O)O ; OC(O)c1ccccc1 ; Cc1cc(-c2ccccc2)cc(F)c1C(=O)O ; O=C(CN1CCC(OC2CCNCC2)CC1)N1CCN(C(=O)c2cc(Cc3n[nH]c(=O)c4ccccc34)ccc2F)CC1 ; Cc1cc(-c2ccccc2)cc(F)c1C(=O)N1CCC(OC2CCN(CC(=O)N3CCN(C(=O)c4cc(Cc5n[nH]c(=O)c6ccccc56)ccc4F)CC3)CC2)CC1]

### Step 1: Synthesis of Intermediate 1-3

To a solution of Intermediate 1-1 (1.5 g, 6.44 mmol, 1 eq) in dioxane (24 mL) was added H₂O (2.4 mL) and Pd(PPh₃)₄ (371.91 mg, 321.84 μmol, 0.05 eq), K₂CO₃ (1.78 g, 12.87 mmol, 2 eq) and Intermediate 1-2 (941.80 mg, 7.72 mmol, 1.2 eq). The system was purged with nitrogen three times. Then the system was allowed to reach 60° C. and stirred for 16 hrs under inert atmosphere. The reaction mixture was concentrated under reduced pressure to remove dioxane (24 mL). Then the residue was diluted with water (150 mL) and acidified by 2N HCl to pH=2. The suspension was filtered, and the solid was filtered off and collected as crude product. The crude product was purified by reverse-phase chromatography (FA condition). The eluent was combined and concentrated under reduced pressure. The residue was then extracted by EA (50 mL*3). The organic phase was then combined and concentrated to afford the target product. After work-up, Intermediate 1-3 (0.77 g, 3.30 mmol, 51.33% yield, 98.8% purity) was obtained as white solid. LCMS (Method E): Retention time=0.508 min, [M+H]⁺=231.1. ¹H NMR (400 MHz, DMSO-d6) δ=13.97-13.09 (m, 1H), 7.77-7.68 (m, 2H), 7.54-7.38 (m, 5H).

### Step 2: Synthesis of I-760

To a solution of Intermediate 1-4 (500.29 mg, 797.72 μmol, 9.42e−1 eq, HCl salt) in DMF (7 mL) was added Intermediate 1-3 (0.195 g, 846.96 μmol, 1 eq), EDCI (487.09 mg, 2.54 mmol, 3 eq), HOAt (115.28 mg, 846.96 μmol, 118.48 μL, 1 eq) and NMM (685.34 mg, 6.78 mmol, 744.94 μL, 8 eq). The resulting mixture was stirred at 25° C. for 1 hr. The reaction mixture was diluted with water (30 mL) and extracted by EA (10 mL*3) to afford the crude. The crude was purified by prep-HPLC (FA condition, column: Phenomenex luna C18 150*25 mm*10 um; mobile phase: [water (FA)-ACN]; gradient: 20%-500% B over 10 min). The eluent was combined and lyophilized to afford the target product. After purification, I-760 (0.155 g, 182.58 μmol, 21.56% yield, 100% purity, FA salt) was obtained as a white solid. LCMS (Method E): Retention time=0.522 min, [M+H]⁺=803.4. ¹H NMR (400 MHz, DMSO-d6) δ=12.59 (s, 1H), 8.26 (d, J=7.6 Hz, 1H), 8.17 (s, 1H), 8.01-7.93 (m, 1H), 7.92-7.78 (m, 2H), 7.72 (d, J=7.2 Hz, 2H), 7.51-7.33 (m, 7H), 7.23 (t, J=9.0 Hz, 1H), 4.33 (s, 2H), 4.15-3.96 (m, 1H), 3.76-3.42 (m, 9H), 3.21-3.04 (m, 6H), 2.73-2.58 (m, 2H), 2.31-2.25 (m, 3H), 2.20-2.03 (m, 2H), 1.94-1.65 (m, 4H), 1.51-1.20 (m, 4H). ¹⁹F NMR (377 MHz, DMSO-d6) δ=−117.25-−118.01 (m, 1F), −119.78 (s, 1F).

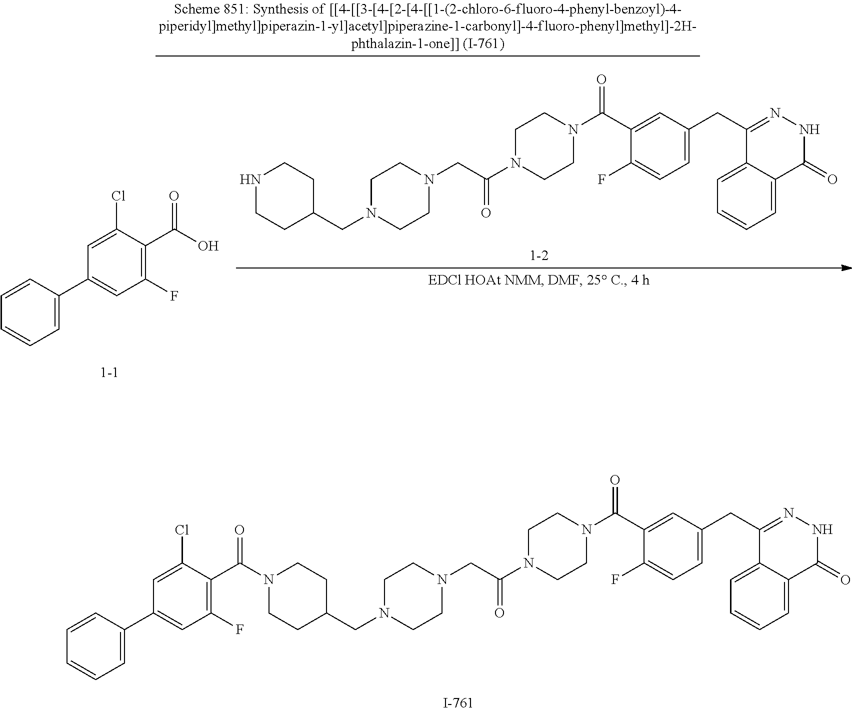 [SMILES: O=C(O)c1c(F)cc(-c2ccccc2)cc1Cl ; O=C(CN1CCN(CC2CCNCC2)CC1)N1CCN(C(=O)c2cc(Cc3n[nH]c(=O)c4ccccc34)ccc2F)CC1 ; O=C(CN1CCN(CC2CCN(C(=O)c3c(F)cc(-c4ccccc4)cc3Cl)CC2)CC1)N1CCN(C(=O)c2cc(Cc3n[nH]c(=O)c4ccccc34)ccc2F)CC1]

To a solution of Intermediate 1-1 (255.03 mg, 1.02 mmol, 1.2 eq) in DMF (5 mL) was added HOAt (57.70 mg, 423.94 μmol, 59.30 μL, 0.5 eq), EDCI (325.08 mg, 1.70 mmol, 2 eq) and NMM (428.81 mg, 4.24 mmol, 466.09 μL, 5 eq), was added Intermediate 1-2 (0.5 g, 847.89 μmol, 1 eq). The mixture was stirred at 25° C. for 12 hr under N₂ atmosphere. The reaction mixture was diluted with H₂O (10 mL) and extracted with EA (10 mL*3). The combined organic layers were washed with brine (10 mL*3), dried over Na₂SO₄, filtered and concentrated under reduced pressure to give a residue. The residue was purified by reversed phase chromatography (105 g of SepaFlash Spherical C18, 20-45 μm, 100 Å; mobile phase: A for H₂O+0.1% NH₃·H₂O and B for acetonitrile; gradient: B 40-60% in 20 min, flow rate: 50 mL/min; column temperature: RT, wavelength: 220 nm/254 nm), and the eluent was concentrated to remove organic solvents and lyophilized to give a crude product. The crude product was re-purified by reversed phase chromatography (105 g of SepaFlash Spherical C18, 20-45 μm, 100 Å; mobile phase: A for H₂O+0.1% FA and B for acetonitrile; gradient: B 30-50% in 20 min, flow rate: 60 mL/min; column temperature: RT, wavelength: 220 nm/254 nm), and the eluent was concentrated to remove organic solvents and lyophilized. I-761 (321.71 mg, 389.77 μmol, 45.97% yield, 99.63% purity, FA) was obtained as a white solid. LCMS (Method E): Rt=13.937 min, [M+H]⁺=822.4. ¹H NMR (400 MHz, DMSO-d6) δ=12.59 (s, 1H), 8.26 (d, J=8.0 Hz, 1H), 8.15 (s, 1H), 7.99-7.94 (m, 1H), 7.92-7.86 (m, 1H), 7.86-7.80 (m, 1H), 7.78 (d, J=7.2 Hz, 2H), 7.73 (d, J=4.0 Hz, 1H), 7.71-7.65 (m, 1H), 7.54-7.41 (m, 4H), 7.40-7.33 (m, 1H), 7.27-7.20 (m, 1H), 4.57-4.45 (m, 1H), 4.33 (s, 2H), 3.70-3.38 (m, 7H), 3.24-3.03 (m, 6H), 2.89-2.78 (m, 1H), 2.44-2.25 (m, 7H), 2.18-2.05 (m, 2H), 1.90-1.60 (m, 3H), 1.16-0.84 (m, 2H).

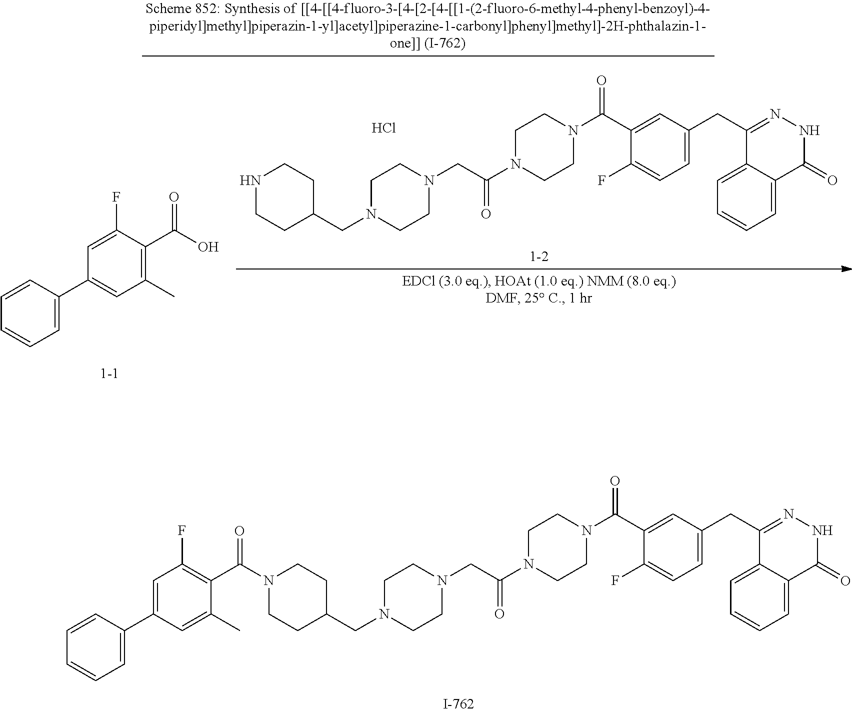 [SMILES: Cc1cc(-c2ccccc2)cc(F)c1C(=O)O ; O=C(CN1CCN(CC2CCNCC2)CC1)N1CCN(C(=O)c2cc(Cc3n[nH]c(=O)c4ccccc34)ccc2F)CC1 ; Cl ; Cc1cc(-c2ccccc2)cc(F)c1C(=O)N1CCC(CN2CCN(CC(=O)N3CCN(C(=O)c4cc(Cc5n[nH]c(=O)c6ccccc56)ccc4F)CC3)CC2)CC1]

A solution of Intermediate 1-2 (HCl salt, 512.26 mg, 868.68 μmol, 1 eq) in DMF (10 mL) was added EDCI (499.58 mg, 2.61 mmol, 3 eq), HOAt (118.24 mg, 868.68 μmol, 121.52 μL, 1 eq), Intermediate 1-1 (0.2 g, 868.68 μmol, 1 eq) and NMM (702.92 mg, 6.95 mmol, 764.04 μL, 8 eq) was stirred at 25° C. for 1 hr. The reaction mixture was diluted by water (50 mL), then extracted by EA (20 mL*3). The organic phase was combined and concentrated under reduced pressure to afford the crude. The crude was purified by prep-HPLC (FA condition, column: Phenomenex luna C18 150*25 mm*10 um; mobile phase: [water (FA)-ACN]; gradient: 18%-48% B over 10 min). The eluent was combined and lyophilized to afford the target product. After purification, I-762 (373.15 mg, 439.35 μmol, 50.58% yield, 99.837% purity, FA salt) was obtained as a white solid. LCMS (Method E): Retention time=0.500 min, [M+H]⁺=802.5. ¹H NMR (400 MHz, DMSO-d6) δ=12.59 (s, 1H), 8.26 (d, J=7.8 Hz, 1H), 8.15 (s, 1H), 7.99-7.94 (m, 1H), 7.92-7.79 (m, 2H), 7.76-7.68 (m, 2H), 7.51-7.34 (m, 7H), 7.27-7.19 (m, 1H), 4.58-4.50 (m, 1H), 4.33 (s, 2H), 3.68-3.39 (m, 7H), 3.22-2.98 (m, 6H), 2.86-2.75 (m, 1H), 2.43-2.22 (m, 10H), 2.17-2.06 (m, 2H), 1.89-1.60 (m, 3H), 1.14-0.78 (m, 2H). ¹⁹F NMR (377 MHz, DMSO-d6) δ=−117.57 (d, J=53.9 Hz, 1F), −119.78 (s, 1F).

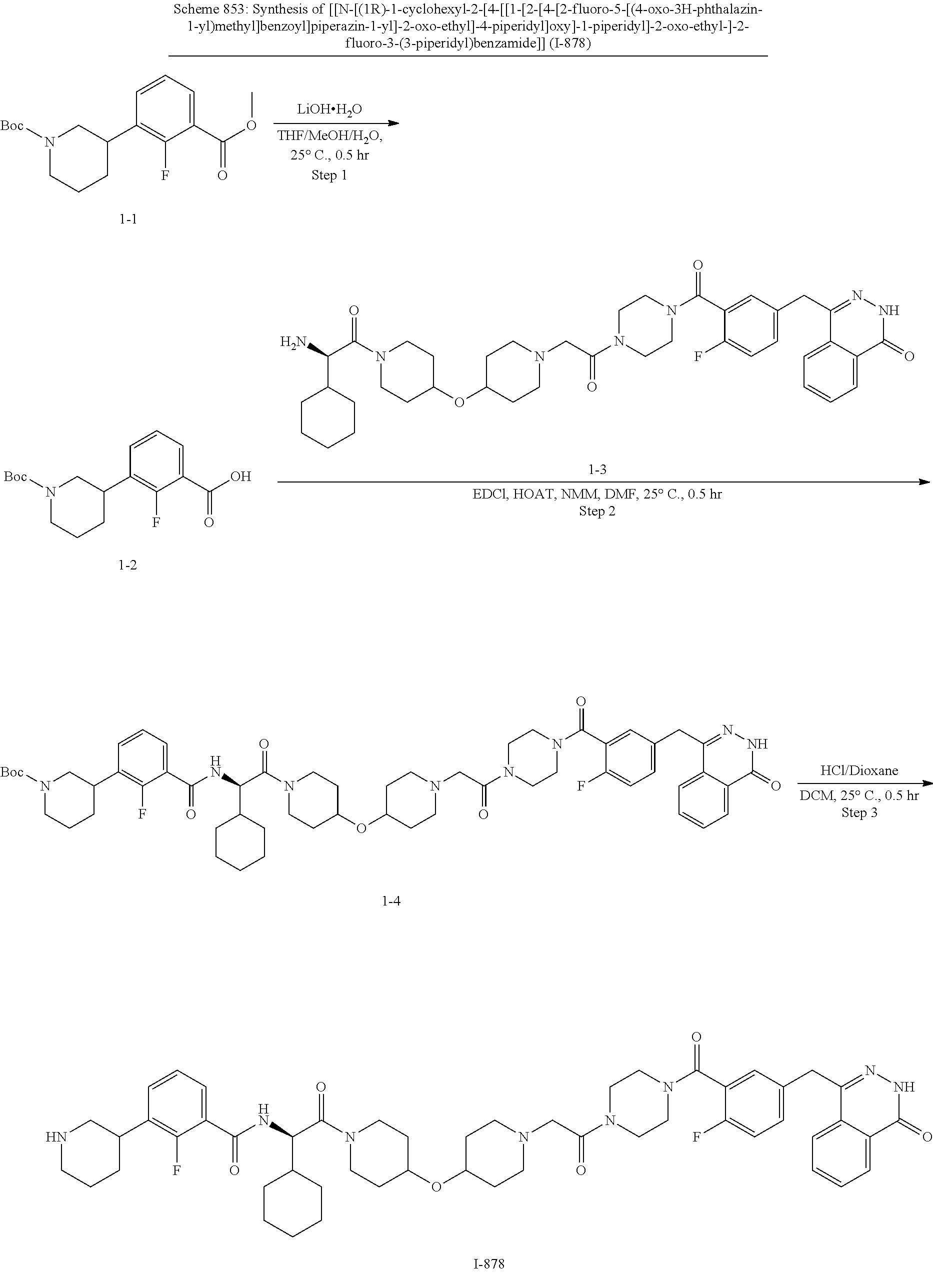

### Step 1: Synthesis of Intermediate 1-2

To a solution of intermediate 1-1 (1 g, 2.96 mmol, 1 eq) in THF (3 mL), MeOH (3 mL) and H₂O (3 mL) was added LiOH H₂O (373.14 mg, 8.89 mmol, 3 eq). The mixture was stirred at 25° C. for 0.5 hr. The reaction mixture was poured into H₂O (5 mL) at 25° C., and adjusted to pH=3 with a 1M CA aqueous solution, and then extracted with DCM (5 mL*3). The combined organic layers were dried over Na₂SO₄, filtered and concentrated under reduced pressure to give a product. Intermediate 1-2 (950 mg, 2.91 mmol, 98.22% yield, 99.093% purity) was obtained as a white solid. LCMS (Method D): Rt=0.377 min, [M+Na]⁺=346.1.

### Step 2: Synthesis of Intermediate 1-4

To a solution of intermediate 1-2 (400 mg, 1.24 mmol, 1 eq) and intermediate 1-3 (947.98 mg, 1.24 mmol, 1 eq, HCl salt) in DMF (4 mL) was added EDCI (711.41 mg, 3.71 mmol, 3 eq), HOAt (168.37 mg, 1.24 mmol, 173.04 μL, 1 eq) and NMM (625.60 mg, 6.19 mmol, 680.00 μL, 5 eq). The mixture was stirred at 25° C. for 0.5 hr. The reaction mixture was poured into H₂O (20 mL), and extracted with EA (5 mL*3). The combined organic layers were dried over Na₂SO₄, filtered and concentrated under reduced pressure to give a residue. The residue was purified by flash silica gel chromatography (ISCO; 20 g SepaFlashSilica Flash Column, Eluent of 0˜100% DCM/MeOH@36 mL/min, DCM/MeOH=10:1, Rf=0.3) and the eluent was concentrated under reduced pressure to give a product. Intermediate 1-4 (1.35 g, crude) was obtained as a white gum. LCMS (Method D): Rt=0.420 min, [M+H]⁺=1036.1.

### Step 3: Synthesis of I-878

To a solution of intermediate 1-4 (500 mg, 482.99 μmol, 1 eq) in DCM (5 mL) was added HCl/dioxane (2 M, 241.49 μL, 1 eq). The mixture was stirred at 25° C. for 0.5 hr. The reaction mixture was concentrated under reduced pressure to give a residue. The residue was purified by reversed phase chromatography (0.10% HCl) and the eluent was concentrated to remove MeCN and then lyophilized to give product. I-878 (213.69 mg, 217.28 μmol, 44.99% yield, 98.79% purity, HCl salt) was obtained as a white solid. LCMS (Method D): Rt=0.319 min, [M+H]⁺=935.5. SCF: Rt=1.864 min, 2.639 min. ¹H NMR (400 MHz, METHANOL-d4) δ=8.36 (d, J=7.6 Hz, 1H), 8.03-7.94 (m, 1H), 7.93-7.80 (m, 2H), 7.60 (t, J=7.2 Hz, 1H), 7.57-7.46 (m, 2H), 7.43-7.36 (m, 1H), 7.29 (t, J=7.6 Hz, 1H), 7.17 (t, J=8.8 Hz, 1H), 4.45-4.30 (m, 4H), 4.08-3.93 (m, 2H), 3.89-3.76 (m, 4H), 3.74-3.64 (m, 2H), 3.55 (s, 3H), 3.50-3.38 (m, 7H), 3.35 (s, 2H), 3.26-3.13 (m, 2H), 3.12-3.03 (m, 1H), 2.31-2.16 (m, 1H), 2.14-2.01 (m, 5H), 2.00-1.90 (m, 3H), 1.86 (d, J=9.6 Hz, 4H), 1.78 (d, J=9.2 Hz, 2H), 1.68 (s, 3H), 1.58 (d, J=13.2 Hz, 1H), 1.34-1.08 (m, 5H). ¹⁹F NMR (377 MHz, METHANOL-d4) δ=−120.650, −121.521, −121.551.

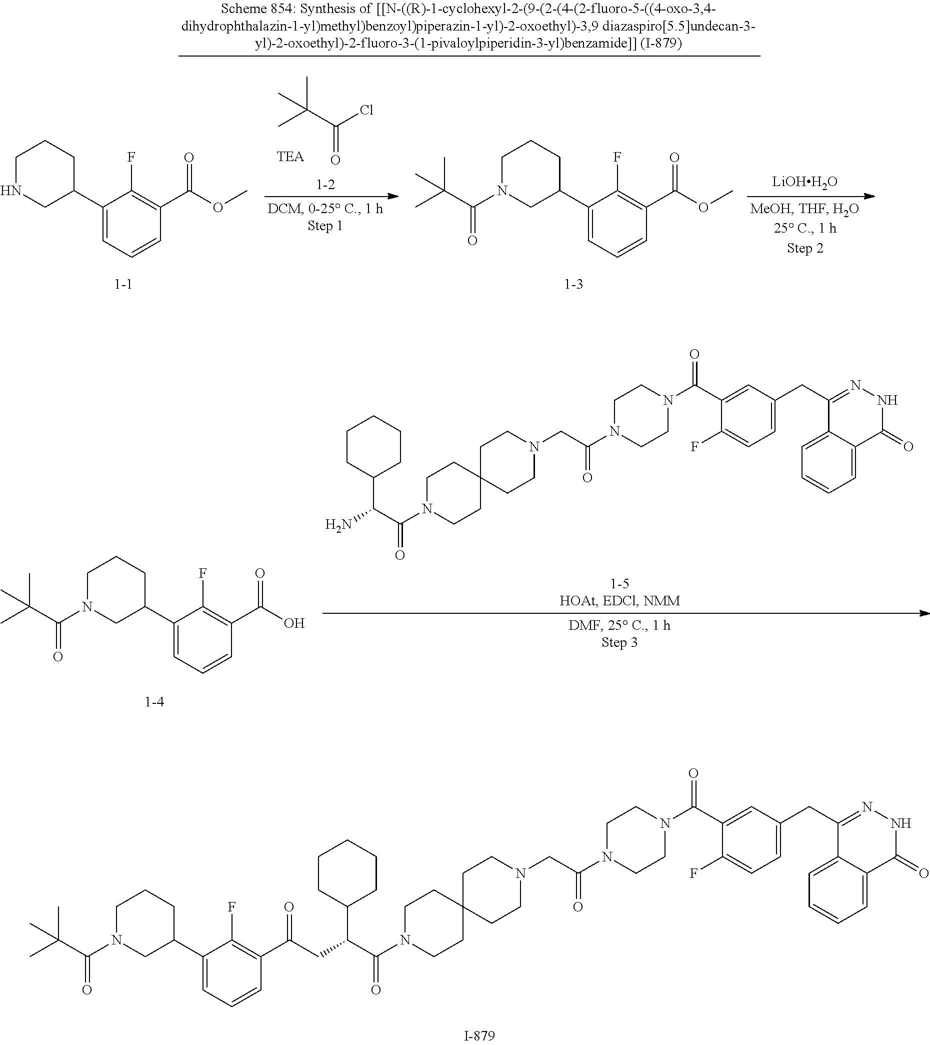

### Step 1: Synthesis of Intermediate 1-2

To a solution of Intermediate 1-1 (1 g, 3.65 mmol, 1 eq, HCl salt) and TEA (1.11 g, 10.96 mmol, 1.53 mL, 3 eq) in DCM (10 mL) was added Intermediate 1-2 (440.50 mg, 3.65 mmol, 449.49 μL, 1 eq) dropwise at 0° C. The mixture was stirred at 25° C. for 1 hr. The mixture was poured into 20 mL of water and extracted with DCM (20 mL*2). The combined organic phase was dried over anhydrous Na₂SO₄, filtered and concentrated in vacuum. The residue was purified by flash silica gel chromatography (ISCO; 40 g SepaFlash Silica Flash Column, Eluent of 0˜30% Ethyl acetate/Petroleum ether gradient @40 mL/min). Intermediate 1-3 (1.05 g, 3.27 mmol, 89.43% yield) was obtained as white solid. LCMS (Method E): Rt=0.626 min, [M+H]⁺=322.1. ¹H NMR (400 MHz, CHLOROFORM-d) δ=7.85-7.78 (m, 1H), 7.46-7.38 (m, 1H), 7.18 (t, J=7.7 Hz, 1H), 4.62-4.38 (m, 2H), 3.94 (s, 3H), 3.15-3.01 (m, 1H), 2.98-2.87 (m, 1H), 2.78 (br t, J=12.4 Hz, 1H), 2.02 (br s, 1H), 1.89-1.73 (m, 2H), 1.70-1.61 (m, 1H), 1.30 (s, 9H).

### Step 2: Synthesis of Intermediate 1-4

To a solution of Intermediate 1-3 (1.05 g, 3.27 mmol, 1 eq) in THF (10 mL), MeOH (10 mL) and H₂O (2 mL) was added LiOH H₂O (274.20 mg, 6.53 mmol, 2 eq). The mixture was stirred at 25° C. for 2 hr. The mixture concentrated in vacuum and the mixture was acidified to pH=2 with 1N HCl, extracted with DCM (10 mL*2), the combined organic phase was dried over anhydrous Na₂SO₄, filtered and concentrated in vacuum. The residue was used directly without further purification. Intermediate 1-4 (770 mg, 2.51 mmol, 76.68% yield) was obtained as white solid. LCMS (Method E): Rt=0.548 min, [M+H]⁺=308.1. ¹H NMR (400 MHz, DMSO-d6) δ=7.94-7.87 (m, 1H), 7.48 (t, J=6.4 Hz, 1H), 7.22 (t, J=7.8 Hz, 1H), 4.54 (br t, J=12.9 Hz, 2H), 3.18-3.04 (m, 1H), 2.99-2.88 (m, 1H), 2.87-2.74 (m, 1H), 2.09-2.03 (m, 1H), 1.90-1.83 (m, 1H), 1.82-1.75 (m, 1H), 1.71-1.62 (m, 1H), 1.32 (s, 9H).

### Step 5: Synthesis of I-879

To a solution of Intermediate 1-4 (208.71 mg, 679.06 μmol, 1 eq) in DMF (10 mL) was added HOAt (184.85 mg, 1.36 mmol, 189.98 μL, 2 eq) and EDCI (260.35 mg, 1.36 mmol, 2 eq). The mixture was stirred at 25° C. for 30 min. Then Intermediate 1-5 (500 mg, 679.06 μmol, 1 eq, HCl salt) and NMM (274.74 mg, 2.72 mmol, 298.63 μL, 4 eq) was added and the mixture was stirred at 25° C. for 1 hr. The reaction was purified directly without further purification. The crude product was purified by reversed-phase chromatography (0.1% FA condition). I-879 (301.97 mg, 290.97 μmol, 42.85% yield, 99.753% purity, HCOOH salt) was obtained as white solid. LCMS (Method E): Rt=0.528 min, [M+H]⁺=989.7. SCF: Rt=0.2.269 min, 3.197 min. ¹H NMR (400 MHz, CHLOROFORM-d) δ=10.72-10.30 (m, 1H), 8.53-8.41 (m, 1H), 8.18 (s, 1H), 7.87 (q, J=7.5 Hz, 1H), 7.83-7.67 (m, 3H), 7.54-7.41 (m, 1H), 7.39-7.30 (m, 3H), 7.24-7.17 (m, 1H), 7.06 (br t, J=8.8 Hz, 1H), 5.10 (br d, J=1.1 Hz, 1H), 4.59-4.42 (m, 2H), 4.30 (s, 2H), 3.85-3.46 (m, 10H), 3.40-3.23 (m, 4H), 3.11-3.03 (m, 1H), 3.00-2.74 (m, 6H), 2.61 (br s, 4H), 2.04 (br d, J=11.6 Hz, 1H), 1.86-1.71 (m, 7H), 1.69-1.60 (m, 4H), 1.49 (br s, 2H), 1.30 (s, 9H), 1.27-1.05 (m, 5H). ¹⁹F NMR (377 MHz, METHANOL-d4) δ=−117.56 (br d, J=31.4 Hz, 1F), −118.73 (br d, J=38.1 Hz, 1F).

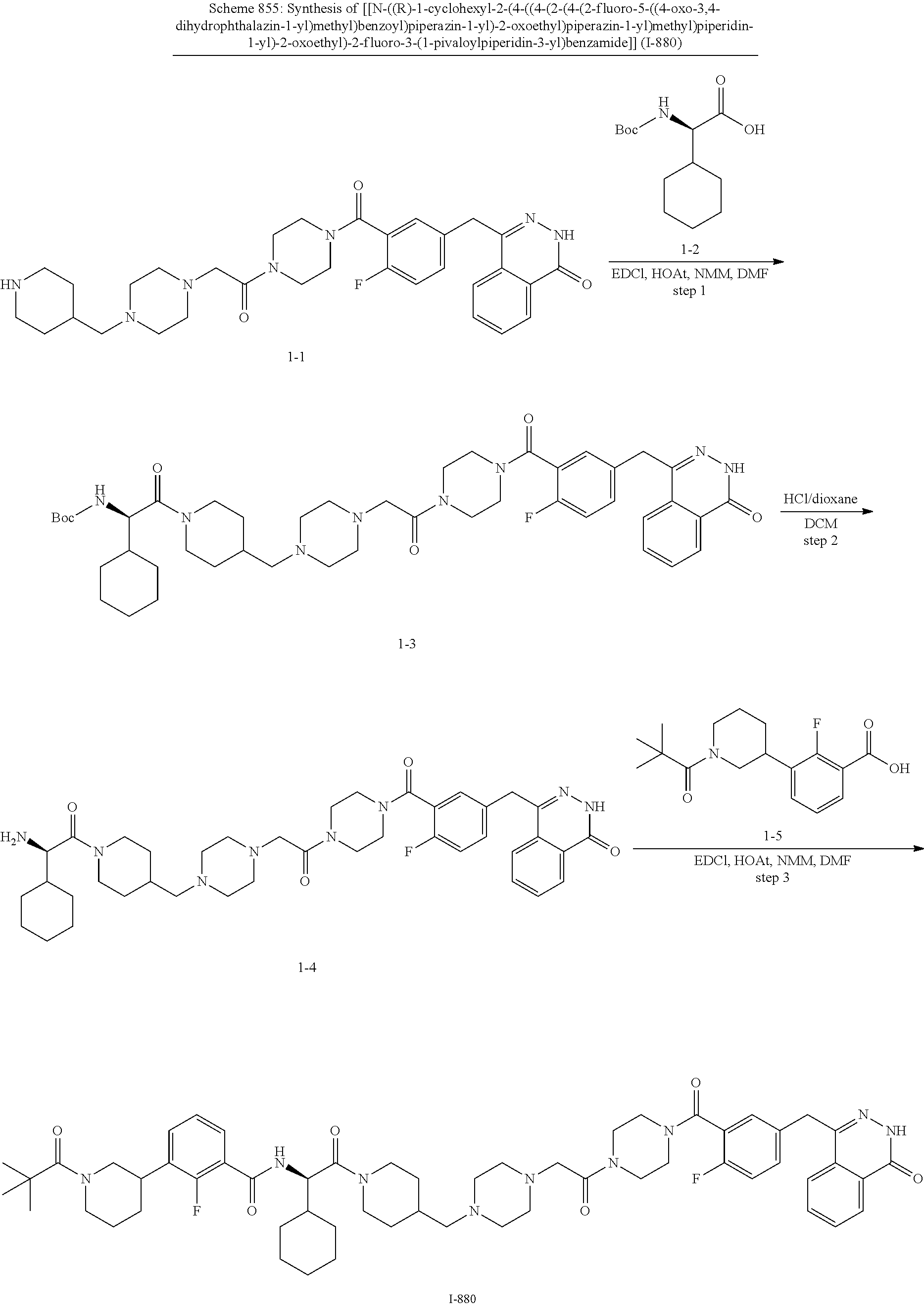

### Step 1: Synthesis of Intermediate 1-3

To a solution of Intermediate 1-1 (600 mg, 958.22 μmol, 1 eq, HCl salt) and Intermediate 1-2 (246.57 mg, 958.22 μmol, 1 eq) in DMF (6 mL) was added HOAt (195.64 mg, 1.44 mmol, 201.06 μL, 1.5 eq), EDCI (367.38 mg, 1.92 mmol, 2 eq) and NMM (484.60 mg, 4.79 mmol, 526.74 μL, 5 eq). The mixture was stirred at 25° C. for 1 hr. The reaction was quenched by water (1 mL) to give a mixture. The mixture was purified by reversed phase HPLC (ISCO; 80 g SepaFlash C18 Column, Eluent of 0˜50% (0.1% FA) water/MeCN@50 mL/min). The combined eluent was concentrated under reduced pressure to remove MeCN and then lyophilized to afford the product. The Intermediate 1-3 (500 mg, 591.07 μmol, 61.68% yield, 98% purity) was obtained as a white solid. LCMS (Method E): Rt=0.481 min, [M+H]⁺=829.6. SCF: Rt=1.469 min. ¹H NMR (400 MHz, CHLOROFORM-d) δ=11.15-10.80 (m, 1H), 8.63-8.36 (m, 1H), 7.86-7.61 (m, 3H), 7.44-7.28 (m, 2H), 7.05 (m, 1H), 5.41 (br d, J=9.2 Hz, 1H), 4.58 (br d, J=12.4 Hz, 1H), 4.46 (br s, 1H), 4.30 (s, 2H), 3.97 (br d, J=12.8 Hz, 1H), 3.91-3.62 (m, 4H), 3.57 (br s, 2H), 3.42-3.12 (m, 4H), 3.10-2.94 (m, 1H), 2.78-2.37 (m, 9H), 2.28 (br d, J=6.4 Hz, 2H), 1.97-1.49 (m, 9H), 1.42 (d, J=3.2 Hz, 9H), 1.29-0.81 (m, 7H). ¹⁹F NMR (377 MHz, CHLOROFORM-d) δ=−117.668.

### Step 2: Synthesis of Intermediate 1-4

To a solution of Intermediate 1-3 (500 mg, 603.13 μmol, 1 eq) in DCM (5 mL) was added HCl/dioxane (4 M, 5 mL, 33.16 eq). The mixture was stirred at 25° C. for 1 hr. The reaction mixture was concentrated to give Intermediate 1-4 (460 mg, 601.03 μmol, 99.65% yield, 100% purity, HCl salt) as a white solid. LCMS (Method E): Rt=0.383 min, [M+H]⁺=729.3.

### Step 3: Synthesis of I-880

To a solution of Intermediate 1-4 (460 mg, 601.03 μmol, 1 eq, HCl salt) and Intermediate 1-5 (184.73 mg, 601.03 μmol, 1 eq) in DMF (5 mL) was added HOAt (122.71 mg, 901.54 μmol, 126.11 μL, 1.5 eq), EDCI (230.43 mg, 1.20 mmol, 2 eq) and NMM (303.96 mg, 3.01 mmol, 330.39 μL, 5 eq). The mixture was stirred at 25° C. for 1 hr. The reaction was quenched by water (1 mL) to give a mixture. The mixture was purified by reversed phase chromatography (ISCO; 80 g SepaFlash C18 Column, Eluent of 0˜95%(0.1% FA) water/MeCN@90 mL/min). The combined eluent was concentrated under reduced pressure to remove MeCN and then lyophilized to give I-880 (350 mg, 328.87 μmol, 54.72% yield, 100% purity, FA salt) as a white solid. LCMS (Method E): Rt=0.509 min, [M+H]⁺=1018.6. SCF: Rt=2.009 min, Rt=2.741 min. ¹H NMR (400 MHz, DMSO-d6) δ=12.60 (s, 1H), 8.43-8.30 (m, 1H), 8.26 (br d, J=8.0 Hz, 1H), 8.14 (d, J=1.2 Hz, 1H), 8.02-7.93 (m, 1H), 7.89 (m, 1H), 7.86-7.79 (m, 1H), 7.55-7.33 (m, 4H), 7.30-7.15 (m, 2H), 4.80 (m, 1H), 4.47-4.24 (m, 5H), 4.18-4.02 (m, 1H), 3.73-3.54 (m, 3H), 3.54-3.42 (m, 2H), 3.36 (br d, J=2.4 Hz, 1H), 3.16 (br s, 3H), 3.12-2.99 (m, 2H), 2.97-2.75 (m, 3H), 2.70-2.55 (m, 2H), 2.47-2.20 (m, 7H), 2.19-2.01 (m, 2H), 1.95-1.38 (m, 14H), 1.21 (s, 9H), 1.14 (br s, 2H), 1.09-0.95 (m, 3H), 0.93-0.74 (m, 1H). ¹⁹F NMR (400 MHz, DMSO-d6) δ=−119.986.

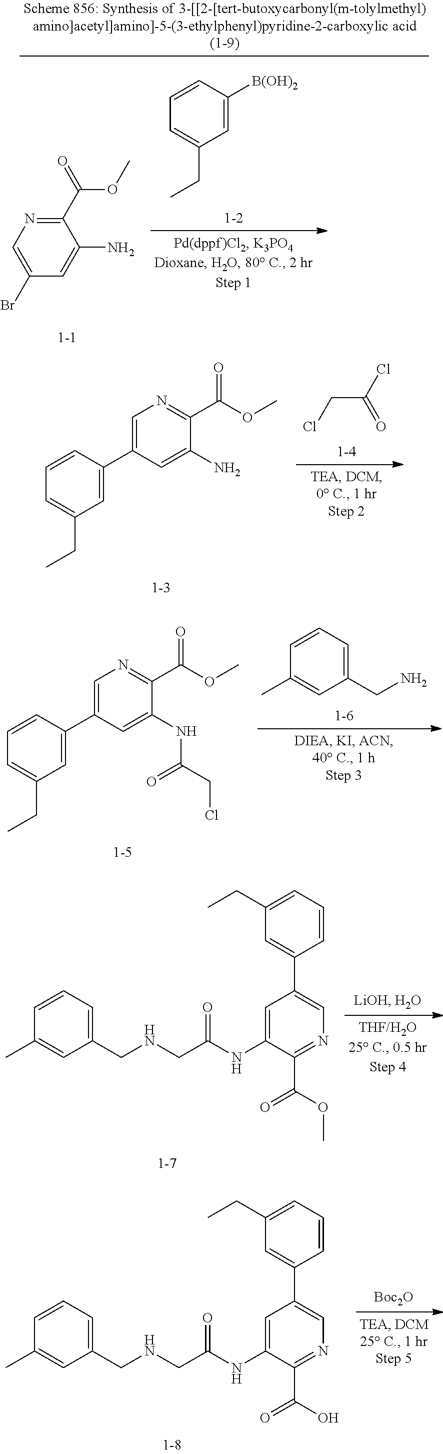

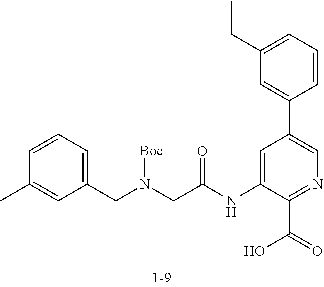 [SMILES: CCc1cccc(-c2cnc(C(=O)O)c(NC(=O)CN(Cc3cccc(C)c3)C(=O)OC(C)(C)C)c2)c1]

### Step 1: Synthesis of Intermediate 1-3

A mixture of Intermediate 1-1 (106 g, 458.78 mmol, 1 eq), Intermediate 1-2 (82.57 g, 550.54 mmol, 1.2 eq), K₃PO₄ (292.15 g, 1.38 μmol, 3 eq), Pd(dppf)Cl₂ (16.78 g, 22.94 mmol, 0.05 eq) in dioxane (1000 mL) and H₂O (200 mL) was degassed and purged with N₂ for 3 times, and then the mixture was stirred at 80° C. for 2 hrs under N₂ atmosphere. The reaction mixture was quenched by addition water 2000 mL at 25° C., and then extracted with EA 600 mL (100 mL*6). Then dried over Na₂SO₄, filtered and concentrated under reduced pressure to give a residue. The residue was purified by flash silica gel chromatography (ISCO®; 330 g SepaFlash® Silica Flash Column, Eluent of 0˜60% Ethyl acetate/Petroleum ether gradient @120 mL/min) to afford Intermediate 1-3 (110 g, 418.41 mmol, 91.20% yield, 97.49% purity) was obtained as a yellow solid. LCMS: Rt=0.458 min, [M+H]⁺=257.0.

### Step 2: Synthesis of Intermediate 1-5

To a solution of Intermediate 1-3 (100 g, 390.17 mmol, 1 eq) in DCM (1000 mL) was added TEA (157.92 g, 1.56 μmol, 217.23 mL, 4 eq) and Intermediate 1-4 (132.20 g, 1.17 μmol, 93.23 mL, 3 eq). The mixture was stirred at 0° C. for 1 hr. The mixture was poured into water (1000 mL) and extracted with DCM (200 mL*6), the combined organic layers were dried over Na₂SO₄, filtered and concentrated under reduced pressure to give a residue. The residue was purified by flash silica gel chromatography (ISCO®; 330 g SepaFlash® Silica Flash Column, Eluent of 0˜60% Ethylacetate/Petroleum ether gradient @120 mL/min) to afford Intermediate 1-5 (127 g, 354.27 mmol, 90.80% yield, 92.83% purity) was obtained as a yellow solid. LCMS: Rt=0.578 min, [M+H]⁺=333.0.

### Step 3: Synthesis of Intermediate 1-7

To a solution of Intermediate 1-5 (40 g, 120.20 mmol, 1 eq) in ACN (400 mL) was added DIEA (62.14 g, 480.80 mmol, 83.75 mL, 4 eq) and Intermediate 1-6 (21.85 g, 180.30 mmol, 22.62 mL, 1.5 eq), KI (9.98 g, 60.10 mmol, 0.5 eq). The mixture was stirred at 40° C. for 1 hr. The reaction mixture was quenched by addition water 1000 mL at 25° C., and then extracted with EA 300 mL (50 mL*6). Then dried over Na₂SO₄, filtered and concentrated under reduced pressure to give a residue. The residue was purified by flash silica gel chromatography (ISCO®; 330 g SepaFlash® Silica Flash Column, Eluent of 0˜80% Ethyl acetate/Petroleum ether gradient @120 mL/min) to afford Intermediate 1-7 (45 g, 94.85 mmol, 78.91% yield, 88% purity) was obtained as a yellow solid. LCMS: Rt=0.479 min, [M+H]⁺=418.1.1

### Step 4: Synthesis of Intermediate 1-8

To a solution of Intermediate 1-7 (46 g, 110.18 mmol, 1 eq) in THF (250 mL), H₂O (250 mL) was added LiOH·H₂O (13.87 g, 330.54 mmol, 3 eq). The mixture was stirred at 25° C. for 0.5 hr. The reaction mixture was concentrated under reduced pressure to give Intermediate 1-8 (45 g, crude) was obtained as yellow solid. LCMS: Rt=0.457 min, [M+H]⁺=404.1.

### Step 5: Synthesis of 1-9

To a solution of Intermediate 1-8 (55 g, 136.32 mmol, 1 eq) in DCM (600 mL) was added TEA (27.59 g, 272.63 mmol, 37.95 mL, 2 eq) and Boc₂O (44.63 g, 204.47 mmol, 46.97 mL, 1.5 eq). The mixture was stirred at 25° C. for 1 hr. The mixture was poured into water (400 mL) and extracted with DCM (100 mL*4), then the pH of aqueous phase was adjusted to 4-5 with 1N HCl, extracted with DCM (100 mL*3), the combined organic layers were dried over Na₂SO₄, filtered and concentrated under reduced pressure to give 1-9 (58 g, 108.84 mmol, 79.84% yield, 94.5% purity) was obtained as a yellow solid. LCMS: Rt=0.646 min, [M+H]⁺=504.3. ¹H NMR (400 MHz, DMSO-d₆) δ=13.36-12.90 (m, 1H), 9.19 (s, 1H), 8.59 (s, 1H), 7.55-7.41 (m, 3H), 7.32 (d, J=7.2 Hz, 1H), 7.25-7.17 (m, 1H), 7.11-7.04 (m, 3H), 4.52 (s, 2H), 4.00-3.89 (m, 2H), 2.69 (m, 2H), 2.27 (s, 3H), 1.41-1.29 (m, 9H), 1.22 (m, 3H).

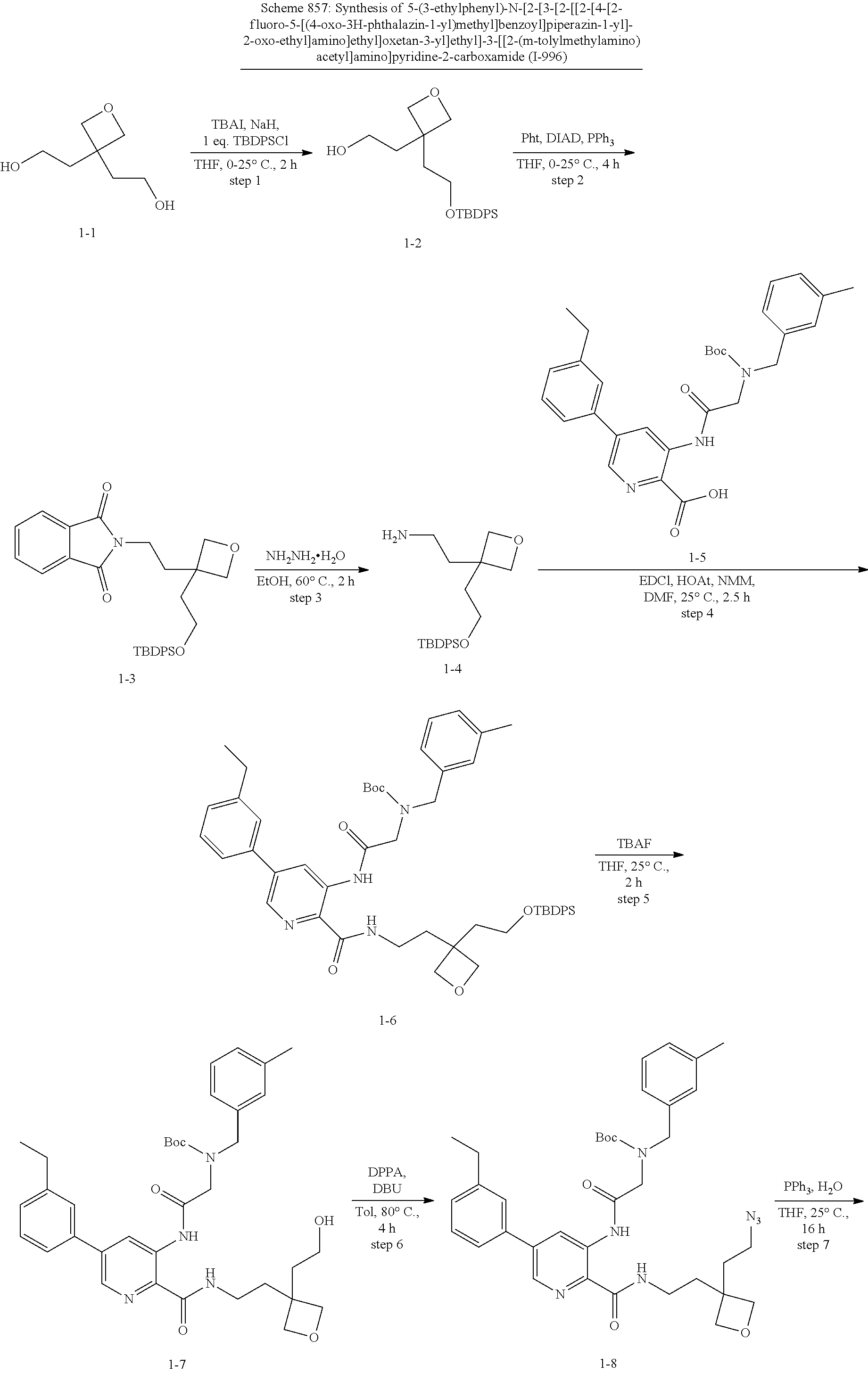

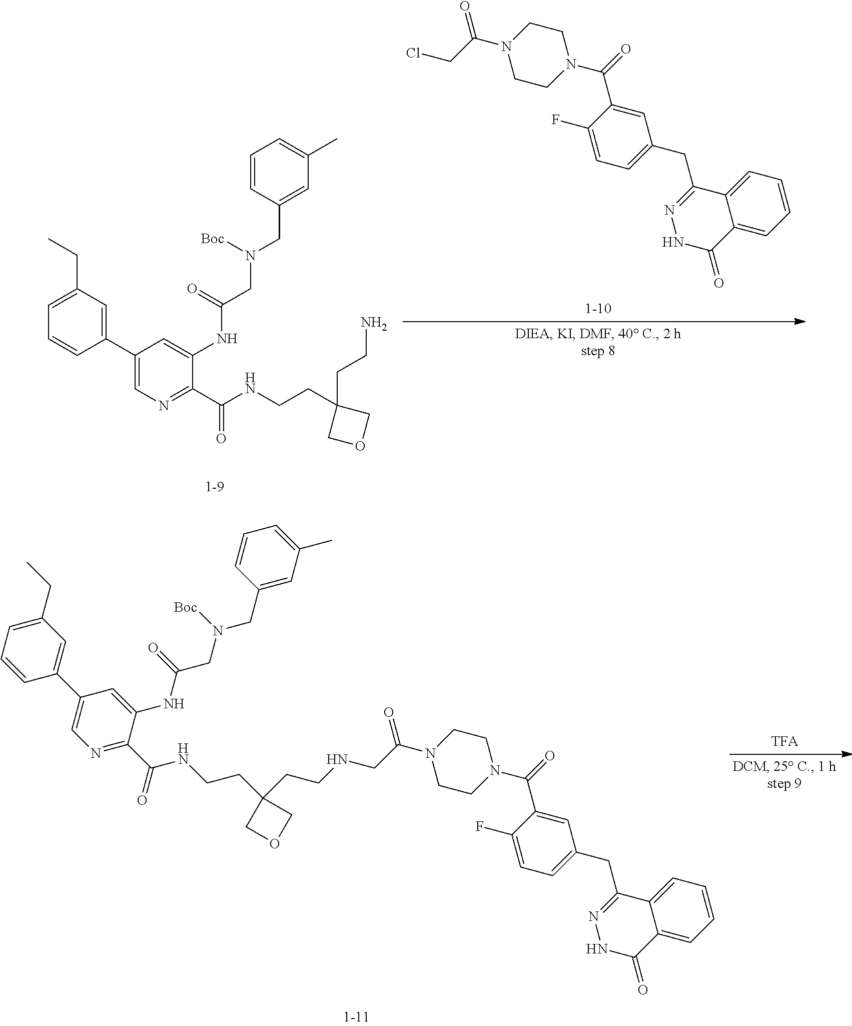 [SMILES: CCc1cccc(-c2cnc(C(=O)NCCC3(CCN)COC3)c(NC(=O)CN(Cc3cccc(C)c3)C(=O)OC(C)(C)C)c2)c1 ; O=C(CCl)N1CCN(C(=O)c2cc(Cc3n[nH]c(=O)c4ccccc34)ccc2F)CC1 ; CCc1cccc(-c2cnc(C(=O)NCCC3(CCNCC(=O)N4CCN(C(=O)c5cc(Cc6n[nH]c(=O)c7ccccc67)ccc5F)CC4)COC3)c(NC(=O)CN(Cc3cccc(C)c3)C(=O)OC(C)(C)C)c2)c1]

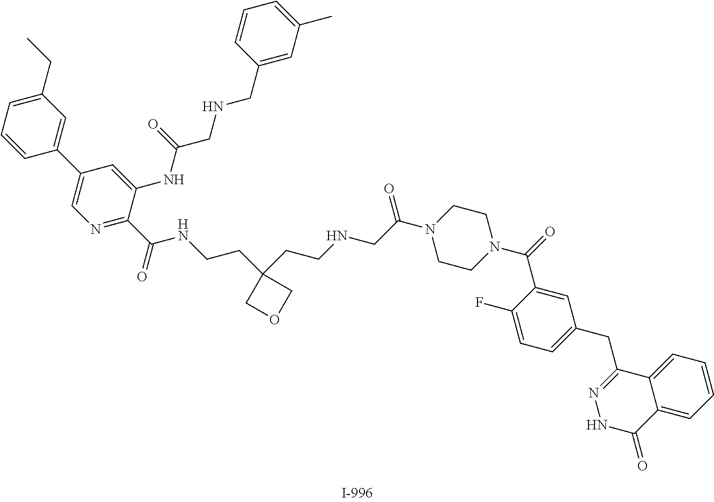 [SMILES: CCc1cccc(-c2cnc(C(=O)NCCC3(CCNCC(=O)N4CCN(C(=O)c5cc(Cc6n[nH]c(=O)c7ccccc67)ccc5F)CC4)COC3)c(NC(=O)CNCc3cccc(C)c3)c2)c1]

### Step 1: Synthesis of Intermediate 1-2

To a solution of Intermediate 1-1 (3 g, 20.52 mmol, 1 eq) in THF (20 mL) was added NaH (1.64 g, 41.04 mmol, 60% purity, 2 eq) at 0° C. Then TBAI (1.52 g, 4.10 mmol, 0.2 eq) and TBDPSCl (5.64 g, 20.52 mmol, 5.25 mL, 1 eq) was added. The mixture was stirred at 25° C. for 2 hr. The mixture was poured into 100 mL aq·NH₄Cl, extracted with EA (100 mL*2). The combined organic phase was dried over anhydrous Na₂SO₄, filtered and concentrated in vacuum. The residue was purified by flash silica gel chromatography (ISCO®; 330 g SepaFlash® Silica Flash Column, Eluent of 0˜50% Ethyl acetate/Petroleum ether gradient @100 mL/min). Intermediate 1-2 (4.2 g, 10.92 mmol, 53.22% yield) was obtained as yellow oil.

¹H NMR (400 MHz, CHLOROFORM-d) δ=7.67 (dd, J=1.6, 7.8 Hz, 4H), 7.51-7.34 (m, 6H), 4.58-4.40 (m, 4H), 3.70 (td, J=6.5, 10.6 Hz, 4H), 2.01 (td, J=6.6, 10.6 Hz, 4H), 1.05 (s, 9H)

### Step 2: Synthesis of Intermediate 1-3

To a solution of Intermediate 1-2 (4 g, 10.40 mmol, 1 eq) and isoindoline-1,3-dione (3.06 g, 20.80 mmol, 2 eq) in THF (80 mL) was added PPh₃ (5.46 g, 20.80 mmol, 2 eq) and DIAD (4.21 g, 20.80 mmol, 4.03 mL, 2 eq). The mixture was stirred at 25° C. for 4 hr. The mixture was concentrated in vacuum. The residue was purified by flash silica gel chromatography (ISCO®; 330 g SepaFlash® Silica Flash Column, Eluent of 0˜50% Ethyl acetate/Petroleum ether gradient @100 mL/min). Intermediate 1-3 (5.5 g, crude) was obtained as white solid. LCMS: Rt=0.715 min, [M+H]⁺=514.4. ¹H NMR (400 MHz, DMSO-d₆) δ=7.87 (dd, J=3.0, 5.4 Hz, 2H), 7.74 (dd, J=3.1, 5.4 Hz, 2H), 7.70-7.64 (m, 4H), 7.46-7.36 (m, 6H), 4.55 (d, J=6.0 Hz, 2H), 4.48 (d, J=6.0 Hz, 2H), 3.81 (t, J=6.3 Hz, 2H), 3.74-3.63 (m, 2H), 2.16-2.02 (m, 4H), 1.04 (s, 9H)

### Step 3: Synthesis of Intermediate 1-4

To a solution of Intermediate 1-3 (5.5 g, 10.71 mmol, 1 eq) To in EtOH (100 mL) was added NH₂NH₂·H₂O (6.1 g, 121.85 mmol, 5.91 mL, 11.38 eq). The mixture was stirred at 60° C. for 2 hr. The mixture was filtered through a pad of celite and the filtrate was concentrated in vacuum. The crude product was purified by reversed-phase HPLC (0.1% FA condition). Intermediate 1-4 (3.5 g, 9.12 mmol, 85.22% yield) was obtained as yellow gum. LCMS: Rt=0.515 min, [M+H]⁺=384.2. ¹H NMR (400 MHz, CHLOROFORM-d) δ=7.70-7.63 (m, 5H), 7.48-7.37 (m, 8H), 4.49 (d, J=5.9 Hz, 2H), 4.41 (d, J=5.9 Hz, 2H), 3.70 (t, J=6.5 Hz, 2H), 2.65-2.57 (m, 2H), 1.95 (t, J=6.5 Hz, 2H), 1.87-1.81 (m, 2H), 1.05 (s, 9H)

### Step 4: Synthesis of Intermediate 1-6

To a solution of Intermediate 1-5 (262.56 mg, 521.38 μmol, 1 eq) in DMF (4 mL) was added HOAt (141.93 mg, 1.04 mmol, 145.87 μL, 2 eq) and EDCI (199.90 mg, 1.04 mmol, 2 eq). The mixture was stirred at 25° C. for 0.5 hr. Then Intermediate 1-4 (200.00 mg, 521.38 μmol, 1 eq) and NMM (158.21 mg, 1.56 mmol, 171.97 μL, 3 eq) was added and the mixture was stirred at 25° C. for 2 hr. The mixture was poured into 20 mL water and extracted with EA (20 mL*2). The combined organic phase was dried over anhydrous Na₂SO₄, filtered and concentrated in vacuum. The residue was purified by flash silica gel chromatography (ISCO®; 20 g SepaFlash® Silica Flash Column, Eluent of 0˜20% Ethyl acetate/Petroleum ether gradient @20 mL/min). Intermediate 1-6 (200 mg, 230.10 μmol, 44.13% yield) was obtained as white oil. LCMS: Rt=0.856 min, [M+H]⁺=869.6. ¹H NMR (400 MHz, CHLOROFORM-d) δ=12.76-12.40 (m, 1H), 9.52-9.30 (m, 1H), 8.45 (br s, 1H), 8.29 (br t, J=5.7 Hz, 1H), 7.67 (dd, J=1.8, 7.6 Hz, 4H), 7.49 (br s, 2H), 7.46-7.37 (m, 7H), 7.30 (br d, J=7.0 Hz, 1H), 7.25-7.18 (m, 1H), 7.13-7.03 (m, 3H), 4.69-4.57 (m, 2H), 4.55 (d, J=6.0 Hz, 2H), 4.47 (br d, J=5.9 Hz, 2H), 4.08 (s, 1H), 3.93 (br s, 1H), 3.76 (t, J=6.2 Hz, 2H), 3.47-3.33 (m, 2H), 2.75 (q, J=7.5 Hz, 2H), 2.33 (s, 3H), 2.13-1.97 (m, 4H), 1.55-1.44 (m, 9H), 1.33-1.29 (m, 4H), 1.05 (s, 9H)

### Step 5: Synthesis of Intermediate 1-7

To a solution of Intermediate 1-6 (200 mg, 230.10 μmol, 1 eq) in THF (3 mL) was added TBAF (1 M, 345.16 μL, 1.5 eq). The mixture was stirred at 25° C. for 2 hr. The mixture was concentrated in vacuum. The residue was purified by flash silica gel chromatography (ISCO®; 12 g SepaFlash® Silica Flash Column, Eluent of 0˜100% Ethyl acetate/Petroleum ether gradient @30 mL/min). Intermediate 1-7 (90 mg, 142.68 μmol, 62.01% yield) was obtained as white gum. LCMS: Rt=0.830 min, [M+H]⁺=631.4. ¹H NMR (400 MHz, CHLOROFORM-d) δ=12.93-12.45 (m, 1H), 9.38 (br s, 1H), 8.50-8.31 (m, 2H), 7.48 (br s, 2H), 7.44-7.38 (m, 1H), 7.33-7.28 (m, 1H), 7.25-7.19 (m, 1H), 7.14-7.05 (m, 3H), 4.63 (br d, J=18.4 Hz, 2H), 4.57-4.44 (m, 4H), 4.12-4.03 (m, 1H), 4.00-3.90 (m, 2H), 3.88-3.81 (m, 1H), 3.57-3.39 (m, 2H), 2.75 (q, J=7.5 Hz, 2H), 2.33 (s, 3H), 2.21-2.06 (m, 4H), 1.57-1.43 (m, 9H), 1.30 (t, J=7.6 Hz, 3H)

### Step 6: Synthesis of Intermediate 1-8

To a solution of Intermediate 1-7 (90 mg, 142.68 μmol, 1 eq) in toluene (2.5 mL) was added DBU (26.07 mg, 171.22 μmol, 25.81 μL, 1.2 eq) and DPPA (47.12 mg, 171.22 μmol, 36.96 μL, 1.2 eq) at 0° C. The mixture was stirred at 80° C. for 4 hr. The mixture was poured into 50 mL water and extracted with EA (20 mL*2). The combined organic phase was dried over anhydrous Na₂SO₄, filtered and concentrated in vacuum. The residue was purified by flash silica gel chromatography (ISCO®; 12 g SepaFlash® Silica Flash Column, Eluent of 0˜100% Ethyl acetate/Petroleum ether gradient @12 mL/min). Intermediate 1-8 (80 mg, 121.99 μmol, 85.50% yield) was obtained as white gum. LCMS: Rt=0.693 min, [M+H]⁺=656.2. ¹H NMR (400 MHz, CHLOROFORM-d) δ=12.51 (br d, J=7.0 Hz, 1H), 9.39 (br d, J=4.5 Hz, 1H), 8.51-8.33 (m, 2H), 7.48 (br s, 2H), 7.44-7.37 (m, 1H), 7.30 (br s, 3H), 7.24-7.19 (m, 1H), 7.15-7.04 (m, 3H), 4.67-4.59 (m, 2H), 4.50 (s, 4H), 4.12-4.05 (m, 1H), 3.94 (br s, 1H), 3.56-3.41 (m, 4H), 2.75 (q, J=7.6 Hz, 2H), 2.33 (s, 3H), 2.12-2.04 (m, 4H), 1.55 (s, 9H), 1.30 (t, J=7.6 Hz, 3H))

### Step 7: Synthesis of Intermediate 1-9

To a solution of PPh₃ (47.99 mg, 182.99 μmol, 1.5 eq) in THF (1 mL) and H₂O (6.59 mg, 365.97 μmol, 6.59 μL, 3 eq) was added a solution of Intermediate 1-8 (80 mg, 121.99 μmol, 1 eq) in THF (1 mL). The mixture was stirred at 25° C. for 16 hr. The mixture was concentrated in vacuum. The crude product was purified by reversed-phase HPLC (0.1% NH₃·H₂O). Intermediate 1-9 (30 mg, 47.64 μmol, 39.05% yield) was obtained as white solid. LCMS: Rt=0.557 min, [M+H]⁺=630.5. ¹H NMR (400 MHz, CHLOROFORM-d) δ=12.75-12.38 (m, 1H), 9.39 (br s, 1H), 8.58-8.30 (m, 2H), 7.74-7.61 (m, 1H), 7.58-7.39 (m, 4H), 7.30 (br d, J=1.3 Hz, 1H), 7.25-7.19 (m, 1H), 7.14-7.03 (m, 3H), 4.63 (br d, J=16.1 Hz, 2H), 4.49 (br s, 4H), 4.16-4.06 (m, 1H), 4.00-3.86 (m, 1H), 3.57-3.41 (m, 2H), 2.92-2.78 (m, 2H), 2.75 (q, J=7.3 Hz, 2H), 2.33 (s, 3H), 2.11-1.92 (m, 4H), 1.56-1.45 (m, 9H), 1.33-1.29 (m, 3H)

### Step 8: Synthesis of Intermediate 1-11

To a solution of Intermediate 1-9 (25 mg, 39.70 μmol, 1 eq), Intermediate 1-10 (15.82 mg, 35.73 μmol, 0.9 eq) and KI (13.18 mg, 79.39 μmol, 2 eq) in DMF (1.5 mL) was added DIEA (10.26 mg, 79.39 μmol, 13.83 μL, 2 eq). The mixture was stirred at 40° C. for 2 hr. The mixture was diluted with MeOH (2 ml) and purified by reversed-phase HPLC (0.1% NH₃·H₂O). Intermediate 1-11 (17 mg, 16.41 μmol, 41.33% yield) was obtained as white solid. LCMS: Rt=0.793 min, [M+H]⁺=1306.5. ¹H NMR (400 MHz, METHANOL-d₄) δ=9.34-9.20 (m, 1H), 8.66-8.49 (m, 1H), 8.37 (br d, J=9.3 Hz, 1H), 7.94-7.82 (m, 3H), 7.54-7.26 (m, 8H), 7.17-7.10 (m, 3H), 4.63 (br s, 1H), 4.54-4.46 (m, 3H), 4.37 (br d, J=14.0 Hz, 2H), 4.07-3.96 (m, 2H), 3.80-3.60 (m, 8H), 3.53-3.40 (m, 8H), 2.76-2.68 (m, 3H), 2.28-2.23 (m, 2H), 2.11-2.00 (m, 4H), 1.54 (br d, J=1.6 Hz, 3H), 1.45-1.36 (m, 6H), 1.31-1.28 (m, 3H)

### Step 9: Synthesis of I-996

To a solution of Intermediate 1-11 (15 mg, 14.48 μmol, 1 eq) in DCM (2 mL) was added TFA (307.00 mg, 2.69 mmol, 0.2 mL, 185.99 eq). The mixture was stirred at 25° C. for 1 hr. The mixture was basified to pH=8 with NH₃·H₂O, concentrated in vacuum. The crude product was purified by reversed-phase HPLC (0.1% NH₃·H₂O). I-996 (10.65 mg, 9.94 μmol, 68.66% yield, 98.00% purity, TFA) was obtained as white solid. LCMS: Rt=0.732 min, [M+H]⁺=936.5. ¹H NMR (400 MHz, METHANOL-d₄) δ =9.37-9.24 (m, 1H), 8.60-8.47 (m, 1H), 8.38-8.32 (m, 1H), 7.95-7.88 (m, 1H), 7.85-7.76 (m, 2H), 7.54-7.39 (m, 4H), 7.36-7.23 (m, 4H), 7.18-7.10 (m, 2H), 7.06-6.94 (m, 1H), 4.57-4.31 (m, 6H), 3.87-3.60 (m, 8H), 3.58-3.36 (m, 10H), 2.76-2.67 (m, 3H), 2.30-2.26 (m, 2H), 2.22-1.87 (m, 4H), 1.30-1.26 (m, 3H). ¹⁹F NMR (377 MHz, METHANOL-d₄) δ=−76.94 (s, 1F), −120.71 (br s, 1F)

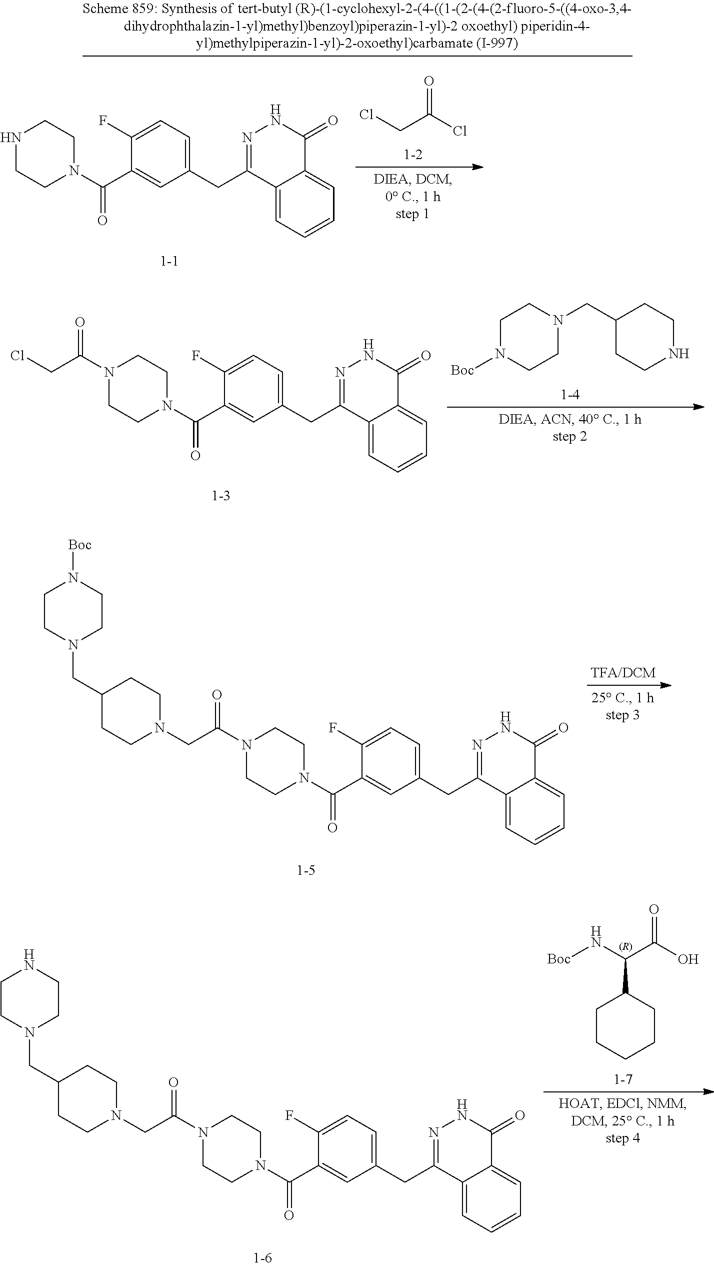

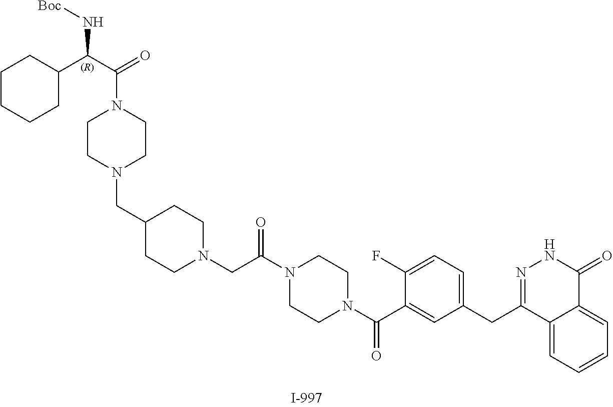

### Step 1: Synthesis of Intermediate 1-3

To a solution of Intermediate 1-1 (36 g, 98.26 mmol, 1 eq) in DCM (360 mL) was added DIEA (63.49 g, 491.28 mmol, 85.57 mL, 5 eq) at 0° C. Then Intermediate 1-2 (14.43 g, 127.73 mmol, 10.17 mL, 1.3 eq) was dropwise added into the mixture and stirred at 0° C. for 1 hr. The reaction mixture was diluted with water (120 mL) and extracted with DCM (40 mL×3). The combined organic layers were washed with brine (40 mL×3) and dried over sodium sulfate, filtered and concentrated under reduced pressure to give a residue. The residue was purified by flash silica gel chromatography (ISCO®; 330 g SepaFlash® Silica Flash Column, Eluent of 0-10% Methanol: Ethyl acetate gradient @100 mL/min). The eluent was concentrated under reduced pressure to give Intermediate 1-3 (26.2 g, 43.78 mmol, 44.55% yield, 74% purity) as a brown gum. LCMS (Method D): Rt: 0.333 min, [M+H]⁺=443.0 ¹H NMR (400 MHz, CHLOROFORM-d) δ=10.78-10.64 (m, 1H), 8.56-8.41 (m, 1H), 7.82-7.70 (m, 3H), 7.34 (d, J=6.0 Hz, 2H), 7.05-7.03 (m, 1H), 4.30 (s, 2H), 3.91-3.72 (m, 2H), 3.72-3.57 (m, 3H), 3.54-3.46 (m, 1H), 3.44-3.28 (m, 2H), 3.11-3.09 (m, 1H), 3.02-2.84 (m, 1H). ¹⁹F NMR (376 MHz, CHLOROFORM-d) δ=−117.642

### Step 2: Synthesis of Intermediate 1-5

To a solution of Intermediate 1-3 (25 g, 56.45 mmol, 1 eq) and Intermediate 1-4 (16.00 g, 56.45 mmol, 1 eq) in ACN (300 mL) was added DIEA (21.89 g, 169.35 mmol, 29.50 mL, 3 eq). The mixture was stirred at 40° C. for 1 h. The mixture was filtered and triturated with ACN (200 mL) and then filtered to give a filter cake which was the pure desired product. The filtrate was concentrated to give a residue and diluted with water (210 mL) and extracted with ethyl acetate (70 mL×3). The combined organic layers were washed with brine (70 mL×3) and dried over sodium sulfate, filtered and concentrated under reduced pressure to give Intermediate 1-5. The combine Intermediate 1-5 (filter cake and extracted product, 38 g) as light yellow solid was used in next step directly. LCMS (Method D): Rt: 0.263 min, [M+H]⁺=690.3

### Step 3: Synthesis of Intermediate 1-6

To a solution of Intermediate 1-5 (20 g, 28.99 mmol, 1 eq) in DCM (200 mL) was added TFA (61.40 g, 538.49 mmol, 40.00 mL, 18.57 eq). The mixture was stirred at 25° C. for 0.5 hr. The mixture was concentrated under reduced pressure to give Intermediate 1-6 (18 g, 25.58 mmol, 88.22% yield, TFA salt) as a yellow oil. LCMS (Method D): Rt: 0.215 min, [M+H]⁺=590.3

### Step 4: Synthesis of I-997

To a solution of Intermediate 1-7 (6.58 g, 25.58 mmol, 1 eq) in DCM (200 mL) was added dropwise EDCI (14.71 g, 76.73 mmol, 3 eq), HOAt (3.48 g, 25.58 mmol, 3.58 mL, 1 eq) and NMM (12.94 g, 127.89 mmol, 14.06 mL, 5 eq) at 25° C. for 0.5 hr. And then Intermediate 1-6 (18 g, 25.58 mmol, 1 eq, TFA salt) was added into mixture and stirred at 25° C. for 0.5 hr. The reaction mixture was diluted with water (120 mL) and extracted with DCM 120 mL (40 mL×3). The combined organic layers were washed with brine (40 mL×3) and dried over sodium sulfate, filtered and concentrated under reduced pressure to give a residue. The residue was purified by prep-HPLC (column: Welch Ultimate XB-Diol 250*50*10 um; mobile phase: [Hexane-EtOH]; gradient: 15%-47% B over 16 min). The eluent was concentrated under reduced pressure to give a residue. I-997 (12.5 g, 10.83 mmol, 42.33% yield, 71.8% purity) was obtained as a white solid. LCMS: Rt: 0.674 min, [M+H]⁺=829.4. ¹H NMR (400 MHz, CHLOROFORM-d) δ=8.50-8.36 (m, 1H), 7.83-7.70 (m, 3H), 7.33 (s, 2H), 7.30-7.043 (m, 1H), 5.28 (d, J=8.0 Hz, 1H), 4.45-4.37 (m, 1H), 4.28 (s, 2H), 4.02-3.47 (m, 13H), 3.44-3.23 (m, 4H), 2.68-2.23 (m, 6H), 2.02-1.91 (m, 2H), 1.80-1.69 (m, 7H), 1.43 (s, 9H), 1.29-0.98 (m, 7H). ¹⁹F NMR (376 MHz, METHANOL-d4) δ=−117.6. SFC: Rt: 1.176

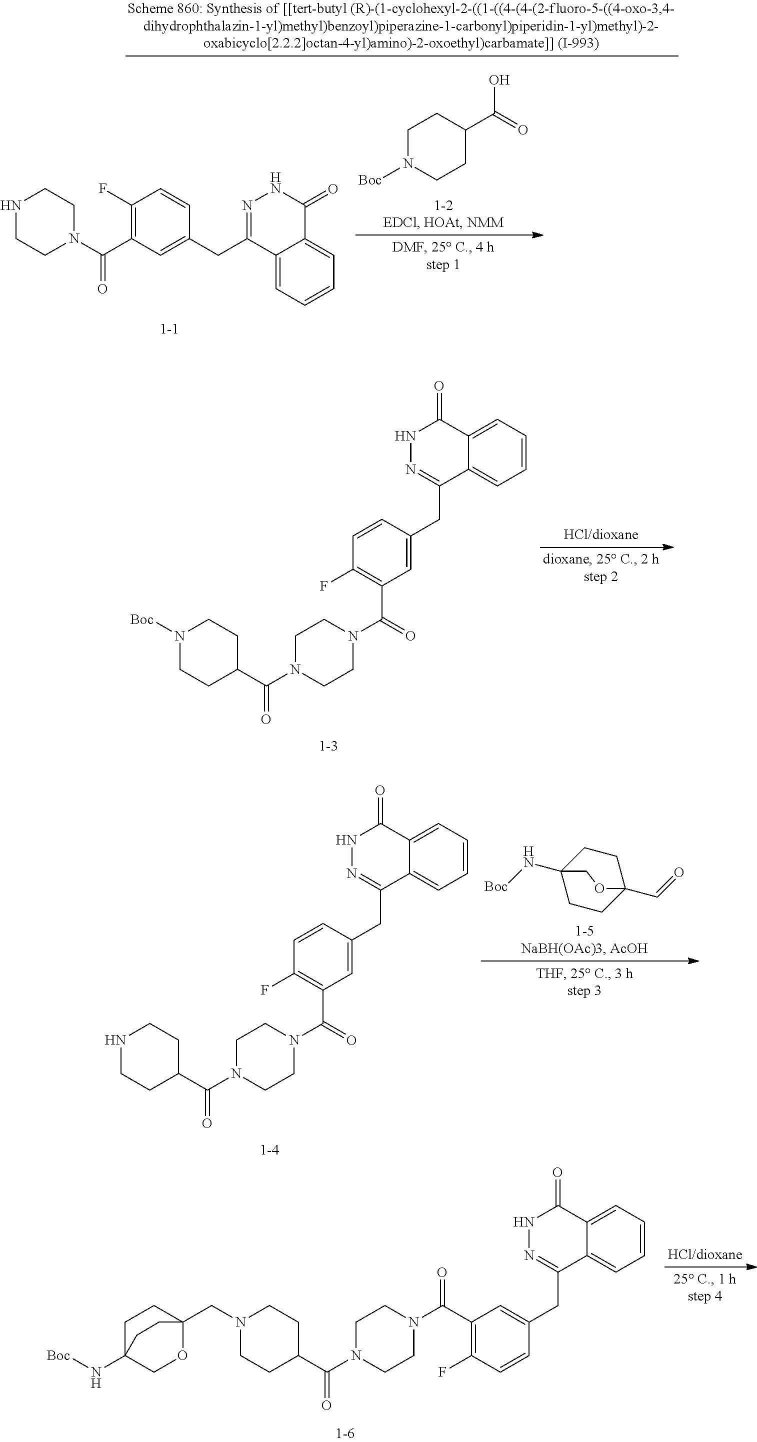

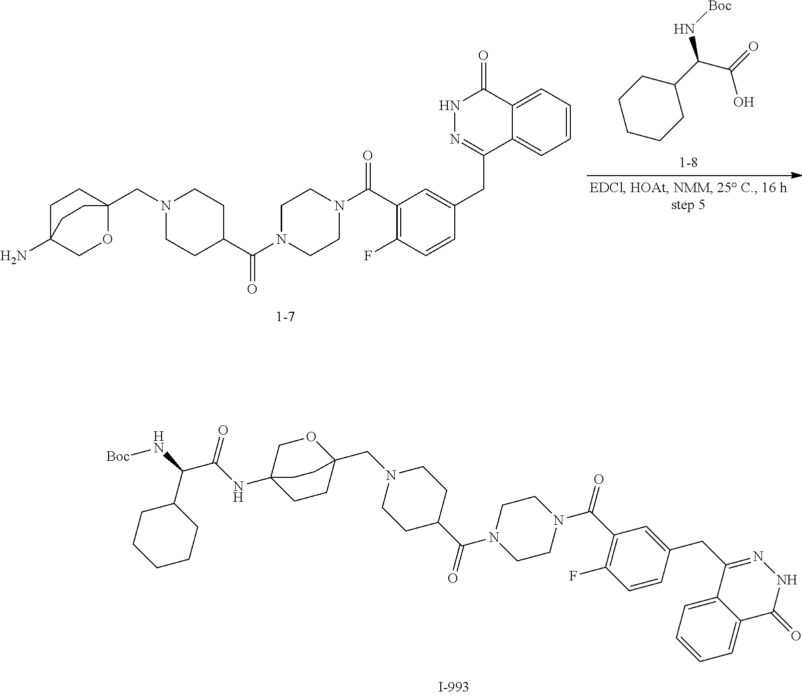 [SMILES: NC12CCC(CN3CCC(C(=O)N4CCN(C(=O)c5cc(Cc6n[nH]c(=O)c7ccccc67)ccc5F)CC4)CC3)(CC1)OC2 ; CC(C)(C)OC(=O)N[C@@H](C(=O)O)C1CCCCC1 ; CC(C)(C)OC(=O)N[C@@H](C(=O)NC12CCC(CN3CCC(C(=O)N4CCN(C(=O)c5cc(Cc6n[nH]c(=O)c7ccccc67)ccc5F)CC4)CC3)(CC1)OC2)C1CCCCC1]

### Step 1: Synthesis of Intermediate 1-3

To a solution of Intermediate 1-2 (750.92 mg, 3.28 mmol, 1.2 eq) in DMF (10 mL) was added HOAt (185.75 mg, 1.36 mmol, 0.5 eq), EDCI (1.05 g, 5.46 mmol, 2 eq), NMM (1.38 g, 13.65 mmol, 1.50 mL, 5 eq) and Intermediate 1-1 (1 g, 2.73 mmol, 1 eq). The mixture was stirred at 25° C. for 4 hr under N₂ atmosphere. The reaction mixture was diluted with H₂O (20 mL) and extracted with EA (20 mL*3). The combined organic layers were washed with brine (15 mL*3), dried over Na₂SO₄, filtered and concentrated under reduced pressure to give a residue. The residue was purified by flash silica gel chromatography (ISCO®; 40 g SepaFlash® Silica Flash Column, Eluent of 20˜30% Ethyl acetate/Methanol gradient, 50 mL/min) to give Intermediate 1-3 (1.7 g, crude) as a white solid. LCMS: Rt=0.483 min, [M+H]⁺=578.5. ¹H NMR (400 MHz, CHLOROFORM-d) δ=10.72-10.53 (m, 1H), 8.50-8.44 (m, 1H), 7.82-7.67 (m, 3H), 7.40-7.29 (m, 2H), 7.09-7.00 (m, 1H), 4.29 (s, 2H), 3.89-3.20 (m, 8H), 2.96 (s, 1H), 2.89 (s, 1H), 2.79-2.57 (m, 2H), 1.77-1.61 (m, 5H), 1.46 (s, 9H).

### Step 2: Synthesis of Intermediate 1-4

To a solution of Intermediate 1-3 (1.7 g, 2.94 mmol, 1 eq) in dioxane (9 mL) was added HCl/dioxane (4 M, 9 mL, 12.23 eq). The mixture was stirred at 25° C. for 2 hr under N₂ atmosphere. The reaction mixture was concentrated under reduced pressure to give Intermediate 1-4 (1.6 g, crude, HCl salt) as a pale green solid. LCMS: Rt=0.473 min, [M+H]⁺=478.2. ¹H NMR (400 MHz, DMSO-d6) δ=12.60 (s, 1H), 9.18-9.01 (m, 1H), 8.84-8.64 (m, 1H), 8.26 (d, J=7.6 Hz, 1H), 7.98-7.94 (m, 1H), 7.93-7.79 (m, 2H), 7.45 (br s, 1H), 7.40-7.31 (m, 1H), 7.28-7.20 (m, 1H), 4.33 (s, 2H), 3.67-3.39 (m, 6H), 3.24 (brd, J=10.8 Hz, 2H), 2.89 (s, 4H), 2.73 (s, 1H), 1.76 (br s, 4H).

### Step 3: Synthesis of Intermediate 1-6

To a solution of Intermediate 1-4 (289.90 mg, 564.02 μmol, 1.2 eq, HCl salt) and Intermediate 1-5 (120 mg, 470.02 μmol, 1 eq) in DCM (5 mL) was added AcOH (2.82 mg, 47.00 μmol, 2.69 μL, 0.1 eq). The mixture was stirred at 25° C. for 2 h, then NaBH(OAc)₃ (298.85 mg, 1.41 mmol, 3 eq) was added and the resulting was stirred at 25° C. for 1 h. The reaction was quenched by aq·NH₄Cl (20 mL) and adjusted to pH=9 with aq·NaHCO₃. The resulting mixture was extracted with DCM (50 mL*2). The combined organic layer was dried over anhydrous Na₂SO₄, filtered and concentrated. The residue was purified by prep-HPLC (FA condition) to give Intermediate 1-5 (250 mg, 289.47 μmol, 61.59% yield, 83% purity) as yellow gum. LCMS: Rt=0.490 min, [M+H]⁺=717.4.

### Step 4: Synthesis of Intermediate 1-7

To a solution of Intermediate 1-6 (50 mg, 69.75 μmol, 1 eq) in HCl/dioxane (2 M, 2 mL, 57.35 eq). The mixture was stirred at 25° C. for 1 h. The reaction was concentrated to give Intermediate 1-7 (40 mg, 55.11 μmol, 79.02% yield, 90% purity, HCl salt) as yellow gum. LCMS: Rt=0.410 min, [M+H]⁺=617.2.

### Step 5: Synthesis of I-993

To a solution of Intermediate 1-7 (40 mg, 61.24 μmol, 1 eq, HCl salt) and Intermediate 1-8 (23.64 mg, 91.86 μmol, 1.5 eq) in DMF (1 mL) was added EDCI (23.48 mg, 122.48 μmol, 2 eq), HOAT (16.67 mg, 122.48 μmol, 2 eq) and NMM (18.58 mg, 183.72 μmol, 20.20 μL, 3 eq). The mixture was stirred at 25° C. for 16 h. The reaction was diluted with water (10 mL) and filtered. The filter cake was washed with water (5 mL) and dried in vacuum to give crude product. The crude product was purified by reversed-phase HPLC (0.1% FA condition), the eluent was lyophilized to give I-993 (17.14 mg, 17.93 μmol, 29.28% yield, 94.36% purity, FA salt) as white solid. LCMS: Rt=0.534 min, [M+H]⁺=856.4. ¹H NMR (400 MHz, CD3OD-d6) δ=8.53 (s, 1H), 8.41-8.33 (m, 1H), 7.99-7.91 (m, 1H), 7.91-7.80 (m, 2H), 7.49 (br s, 1H), 7.37 (br t, J=5.6 Hz, 1H), 7.16 (t, J=8.8 Hz, 1H), 4.94-4.89 (m, 1H), 4.84 (br s, 1H), 4.38 (s, 2H), 3.99 (q, J=7.8 Hz, 2H), 3.83-3.63 (m, 5H), 3.51 (br s, 2H), 3.24 (br d, J=10.2 Hz, 2H), 2.85-2.37 (m, 5H), 2.24-2.09 (m, 2H), 2.06-1.82 (m, 6H), 1.80-1.52 (m, 10H), 1.43 (s, 9H), 1.30-1.15 (m, 3H), 1.11-0.94 (m, 2H). ¹⁹F NMR (400 MHz, CD3OD-d6) δ=−120.75 (s, 1F). SFC: Rt=0.924 min

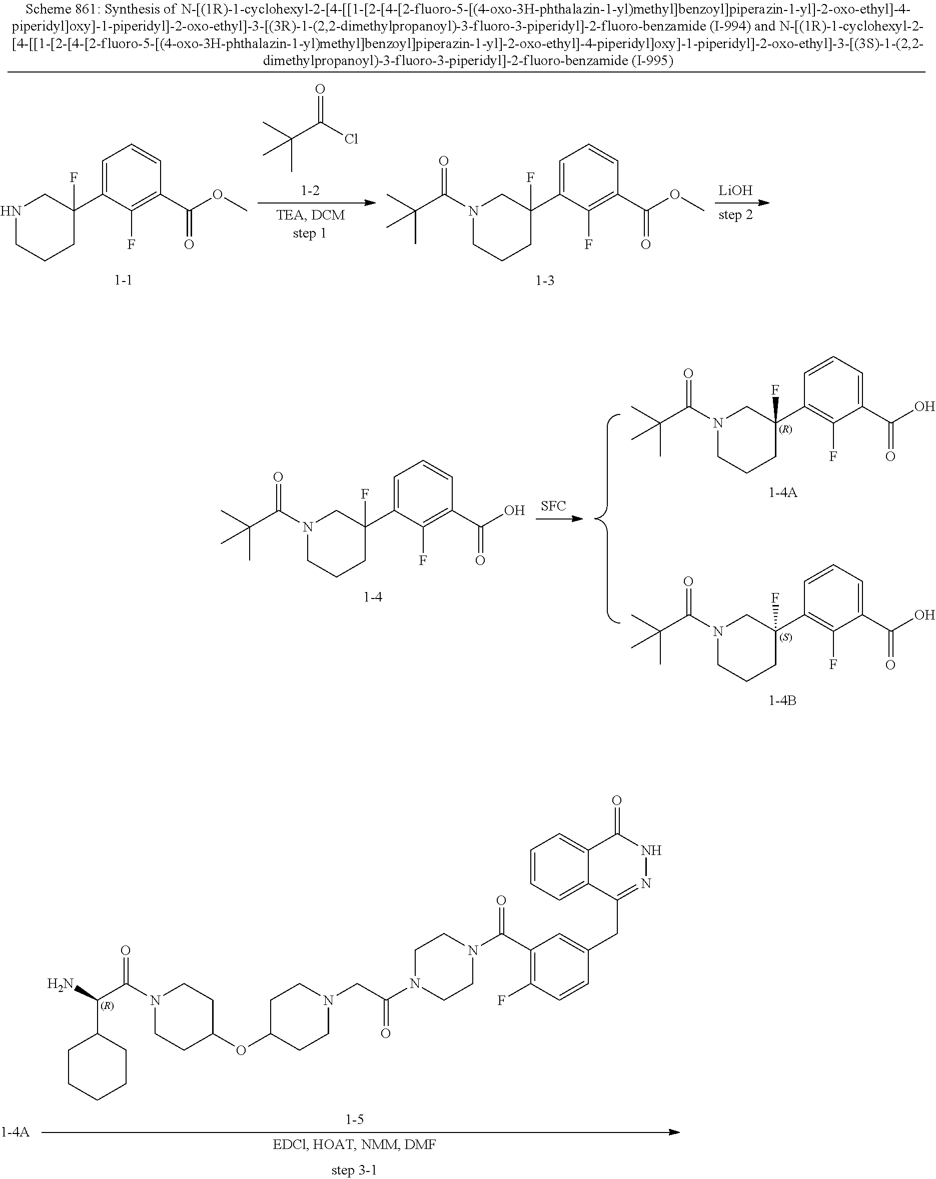

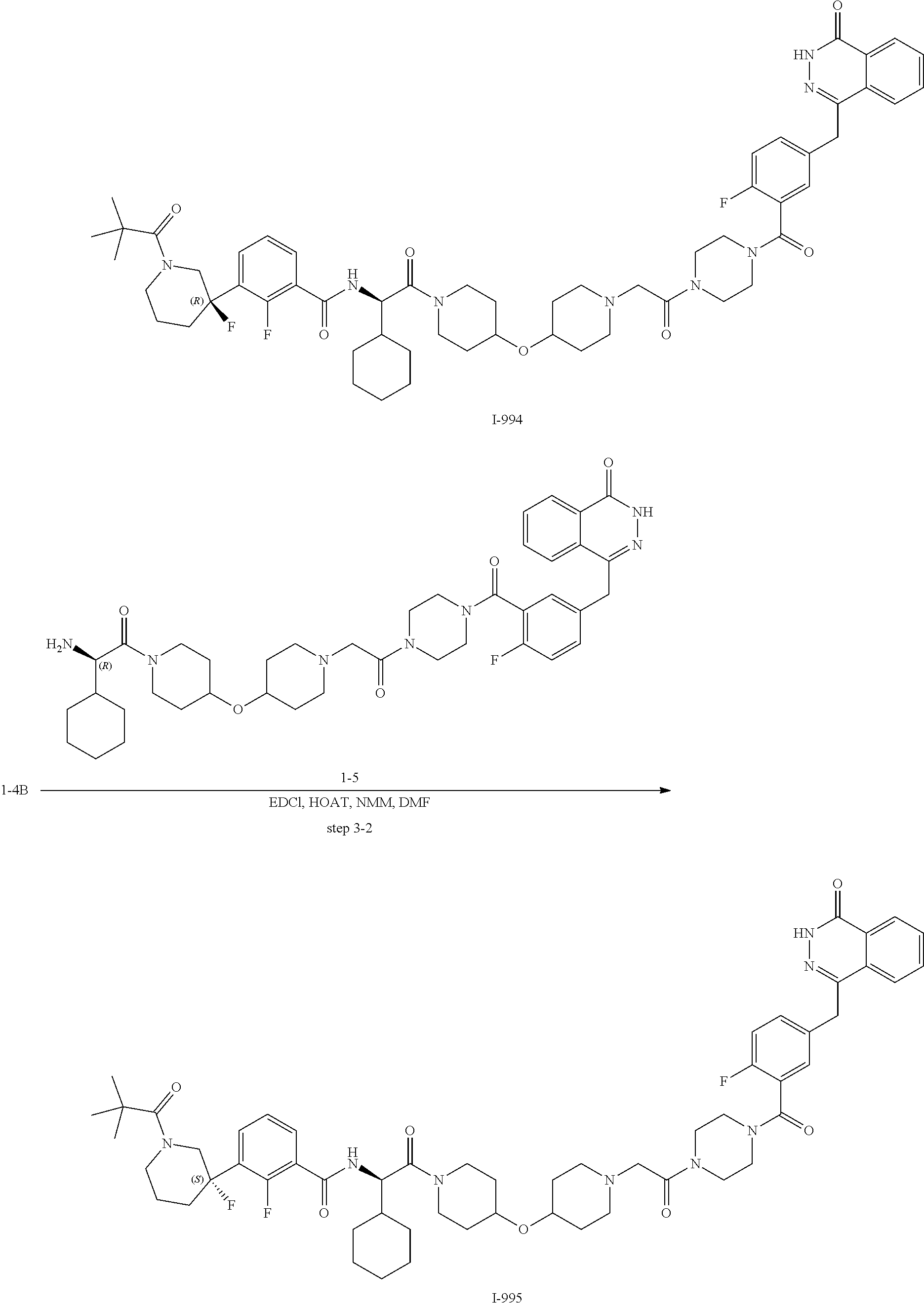

### Step 1: Synthesis of Intermediate 1-3

To a solution of intermediate 1-1 (500 mg, 1.71 mmol, 1 eq, HCl salt) and intermediate 1-2 (227.33 mg, 1.89 mmol, 231.97 μL, 1.1 eq) in DCM (5 mL) was added TEA (260.15 mg, 2.57 mmol, 357.84 L, 1.5 eq). The mixture was stirred at 20° C. for 12 hr. The reaction mixture was extracted with DCM (3 mL*3). The combined organic layers were washed with brine (5 mL*2), dried over Na₂SO₄, filtered and concentrated under reduced pressure to give a residue. The residue was purified by flash silica gel chromatography (ISCO®; 20 g SepaFlash® Silica Flash Column, Eluent of 0˜50% Ethyl acetate/Petroleum ethergradient @20 mL/min). The eluent was concentrated to afford intermediate 1-3 (460 mg, 1.36 mmol, 65.90% yield, 100% purity) as a white solid. LCMS: Rt=0.553 min, [M+H]⁺=340.2

### Step 2: Synthesis of Intermediate 1-4A and Intermediate 1-4B

To a solution of Intermediate 1-3 (460 mg, 1.36 mmol, 1 eq) in THF (2 mL) was added LiOH·H₂O (113.76 mg, 2.71 mmol, 2 eq) in H₂O (2 mL) and MeOH (2 mL). The mixture was stirred at 25° C. for 1 hr. The reaction was acidify with 0.5M aq·HCl to pH<7, and extracted with EtOAc (3 mL*3). The combined organic layers were dried over Na₂SO₄, filtered and concentrated under reduced pressure to give a residue. The residue was purified by SFC (column: DAICEL CHIRALPAK IC (250 mm*30 mm, 10 um);mobile phase: [CO₂-EtOH (0.1% NH3H2O)]; B %: 45%, isocratic elution mode). The eluent was concentrated to get the residue and checked by SFC. Then the residue was separated by SFC (column: DAICEL CHIRALCEL OJ (250 mm*30 mm, 10 um);mobile phase: [CO₂-MeOH (0.1% NH₃H₂O)]; B %: 10%, isocratic elution mode) to give peak 1 (Intermediate 1-4A, Rt=0.731 min) and peak 2 (Intermediate 1-4B, Rt=0.980 min). Intermediate 1-4A (200 mg, 614.72 μmol, 45.35% yield) was obtained as a white solid. Intermediate 1-4B (200 mg, 614.72 μmol, 45.35% yield) was obtained as a white solid. CMS: Rt=0.482 min, [M+H]⁺=326.1 SFC: Rt=1.382 min. SFC: Rt=0.751, 0.995 min. SFC: Rt=0.731 min. SFC: Rt=0.980 min

### Step 3-1: Synthesis of I-994

To a solution of intermediate 1-4A (30 mg, 92.21 μmol, 1 eq) and intermediate 1-5 (77.73 mg, 101.43 μmol, 1.1 eq, HCl salt) in DMF (1 mL) was added EDCI (53.03 mg, 276.63 μmol, 3 eq), NMM (74.61 mg, 737.67 μmol, 81.10 μL, 8 eq) and HOAt (12.55 mg, 92.21 μmol, 12.90 μL, 1 eq). The mixture was stirred at 25° C. for 1 hr. The mixture was concentrated under the pressure to give residue. The residue was purified by prep-HPLC (column: Phenomenex luna C18 150*40 mm*15 um; mobile phase: [water (FA)-ACN]; gradient: 25%-55% B over 10 min). The eluent was lyophilized to afford I-994 (33 mg, 30.46 μmol, 33.04% yield, 100% purity, FA salt) as a white solid. LCMS: Rt=0.520 min, [M+H]⁺=1037.6

SFC: Rt=2.554 min. ¹H NMR (400 MHz, DMSO-d6) δ=12.79-12.43 (m, 1H), 8.69-8.46 (m, 1H), 8.38-8.22 (m, 1H), 8.14 (s, 1H), 8.08-7.77 (m, 3H), 7.70-7.51 (m, 2H), 7.50-7.19 (m, 4H), 4.85-4.52 (m, 2H), 4.46-4.26 (m, 3H), 4.02-3.82 (m, 2H), 3.70-3.55 (m, 4H), 3.54-3.43 (m, 3H), 3.42-3.36 (m, 3H), 3.29 (br s, 2H), 3.24-2.93 (m, 6H), 2.75-2.60 (m, 2H), 2.33-2.02 (m, 4H), 1.91 (br d, J=8.0 Hz, 10H), 1.64-1.52 (m, 2H), 1.47-1.34 (m, 3H), 1.21 (s, 9H), 1.17-0.87 (m, 4H). ¹⁹F NMR (377 MHz, DMSO-d6) δ=−114.77 (br dd, J=10.1, 19.1 Hz, 1F), −119.77 (br s, 1F), −157.94-−158.20 (m, 1F)

### Step 3-2: Synthesis of I-995

To a solution of Intermediate 1-4B (30 mg, 92.21 μmol, 1 eq) and intermediate 1-5 (77.73 mg, 101.43 μmol, 1.1 eq, HCl salt) in DMF (1 mL) was added EDCI (53.03 mg, 276.63 μmol, 3 eq), NMM (74.62 mg, 737.67 μmol, 81.10 μL, 8 eq) and HOAt (12.55 mg, 92.21 μmol, 12.90 μL, 1 eq). The mixture was stirred at 25° C. for 1 hr. The mixture was concentrated under the pressure to give residue. The residue was purified by prep-HPLC (column: Phenomenex luna C18 150*25 mm*10 um; mobile phase: [water (FA)-ACN]; gradient: 25%-55% B over 10 min). I-995 (34 mg, 31.39 μmol, 34.04% yield, FA salt) as a white solid. LCMS: Rt=0.513 min, [M+H]⁺=1037.6. SFC: Rt=1.748 min. ¹H NMR (400 MHz, DMSO-d6) δ=12.59 (s, 1H), 8.61-8.48 (m, 1H), 8.33-8.23 (m, 1H), 8.14 (s, 1H), 8.01-7.94 (m, 1H), 7.94-7.78 (m, 2H), 7.62-7.50 (m, 2H), 7.47-7.35 (m, 2H), 7.34-7.20 (m, 2H), 4.81-4.56 (m, 2H), 4.42-4.30 (m, 3H), 4.01-3.85 (m, 2H), 3.71-3.56 (m, 4H), 3.52-3.43 (m, 3H), 3.41-3.35 (m, 3H), 3.29-3.23 (m, 2H), 3.19-2.94 (m, 6H), 2.71-2.60 (m, 2H), 2.34-2.04 (m, 4H), 1.85-1.65 (m, 10H), 1.59 (br d, J=11.2 Hz, 2H), 1.47-1.34 (m, 3H), 1.21 (s, 9H), 1.16-1.00 (m, 4H) [3362]¹⁹F NMR (377 MHz, DMSO-d6) δ=−114.34-−114.82 (m, 1F), −119.77 (br s, 1F), −158.05 (br dd, J=11.2, 33.7 Hz, 1F)

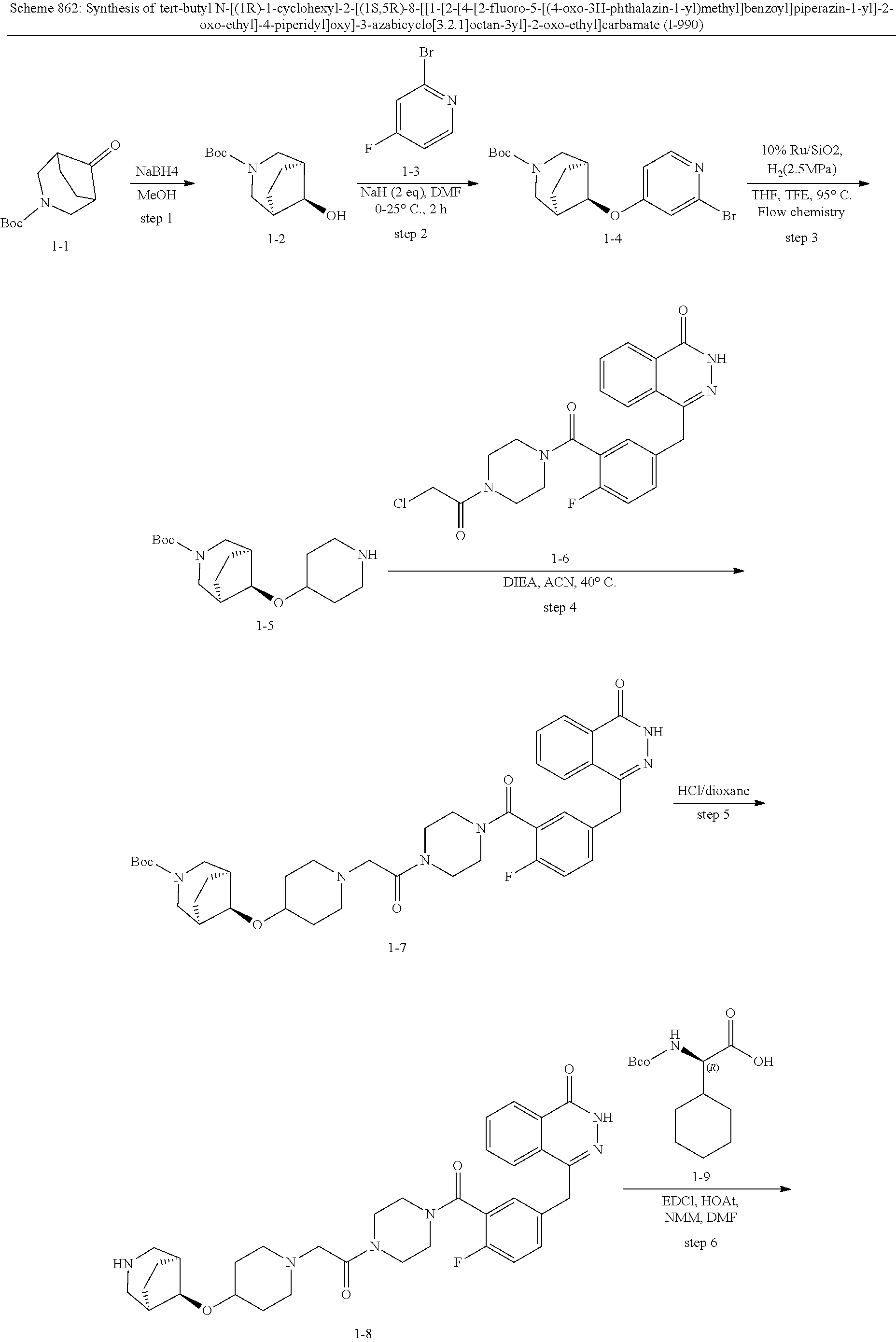

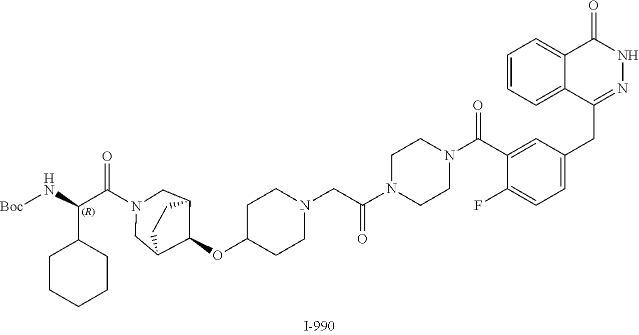

### Step 1: Synthesis of Intermediate 1-2

To a solution of Intermediate 1-1 (9 g, 39.95 mmol, 1 eq) in MeOH (90 mL) was added NaBH₄ (2.27 g, 59.92 mmol, 1.5 eq) at 0° C. Then the mixture was stirred at 0° C. for 1 hr. The reaction mixture was poured into water (200 mL) and extracted with EA (200 mL*2), the organic layer was washed with brine (200 mL) and dried over Na₂SO₄, then concentrated to get Intermediate 1-2 (9 g, 39.60 mmol, 99.11% yield, 100% purity) as white solid without further purification. LCMS: Rt=0.469 min, [M+Na]⁺=172.2. ¹H NMR (400 MHz, DMSO-d6) δ=4.06-4.04 (m, 1H), 3.70 (br d, J=11.6 Hz, 1H), 3.56 (br d, J=12.0 Hz, 1H), 3.43-3.26 (m, 2H), 2.06-1.95 (m, 2H), 1.75-1.57 (m, 4H), 1.48 (s, 9H)

### Step 2: Synthesis of Intermediate 1-4

To a solution of Intermediate 1-2 (5 g, 22.00 mmol, 1 eq) in DMF (50 mL) was added NaH (1.76 g, 43.99 mmol, 60% purity, 2 eq) under N₂ atmosphere. The mixture was stirred at 0° C. for 0.5 h, then Intermediate 1-3 (5.81 g, 33.00 mmol, 1.5 eq) was added to the reaction mixture and the mixture was stirred at 25° C. for 1.5 h under N₂. The reaction mixture was quenched by saturated NH₄C1, extracted with EA (50 mL*3). The combined organic layers were washed with brine (50 mL*3), dried over anhydrous Na₂SO₄, filtered and concentrated under reduced pressure to give a residue. The residue was triturated with methyl tert-butyl ether (50 mL) for 20 min, then filtered off to collect the white solid, the white solid was dried under vacuum to give Intermediate 1-4 (6.4 g, 16.53 mmol, 75.15% yield, 99% purity) as a white solid.

LCMS: Rt=0.599 min, [M+H]⁺=385.2. ¹HNMR (400 MHz, DMSO-d6) δ=8.19 (d, J=6.0 Hz, 1H), 7.38 (d, J=2.0 Hz, 1H), 7.16-7.14 (m, 1H), 4.73-4.71 (m, 1H), 3.65-3.47 (m, 2H), 3.18 (d, J=12.8 Hz, 1H), 3.04 (d, J=12.4 Hz, 1H), 2.37-2.25 (m, 2H), 1.84-1.72 (m, 2H), 1.51-1.44 (m, 2H), 1.39 (s, 9H). SFC: Rt=1.516 min, ee value>99%

### Step 3: Synthesis of Intermediate 1-5

Solution 1: {Intermediate 1-4, 1 eq, 6.3 g} and {TEA, 2 eq, 3.327 g} in {THF, 63 mL} and {TFE, 63 mL}. The fixed bed (named FLR1, volume 20 mL) was completely packed with granular catalyst 10% Ru/SiO₂ (1.00 eq, 10 g). The H₂ back pressure regulator was adjusted to 2.5 MPa, and the flow rate of H₂ was 60 mL/min. Then the solution S1 was pumped by Pump 1 {S1, P1, 1.212 mL/min} to fixed bed {FLR1, SS, Fixed bed, 9.525(½″) mm, 4 mL, 95° C.}. The solution S1 was flowing through {FLR1, 3.3 min} to leave the reactor zone, then the reaction mixture was collected from the reactor output. The mixture was concentrated to give Intermediate 1-5 (4.5 g, crude) as a white solid. ¹HNMR (400 MHz, DMSO-d6) δ=8.75-8.23 (m, 2H), 3.77-3.66 (m, 2H), 3.57-3.41 (m, 2H), 3.24-3.13 (m, 3H), 3.12-3.04 (m, 2H), 3.03-2.92 (m, 2H), 2.10-1.90 (m, 3H), 1.77-1.52 (m, 4H), 1.38 (s, 9H), 1.18 (m, 2H).

### Step 4: Synthesis of Intermediate 1-7

To a mixture of Intermediate 1-5 (250.00 mg, 805.33 μmol, 1 eq) in CH₃CN (4 mL) was added Intermediate 1-6 (356.66 mg, 805.33 μmol, 1 eq) and DIPEA (312.25 mg, 2.42 mmol, 420.82 μL, 3 eq) at 20° C. The mixture was stirred at 40° C. for 12 hrs. The reaction mixture was poured into water (10 mL), then extracted with EA (10 mL*3), the organic layer was washed with brine (10 mL), dried over Na₂SO₄, filtered and concentrated to get the crude product. The crude product was purified by reversed phase (FA condition), the eluent was concentrated to remove organic solvents. The residual aqueous solution was extracted with DCM (50 mL*3), the organic layer was washed with brine (100 mL), dried over Na₂SO₄, filtered and concentrated to give Intermediate 1-7 (330 mg, 437.34 μmol, 54.31% yield, 95% purity) as a white solid. LCMS: Retention time: 0.470 min, [M+H]⁺=717.3

### Step 5: Synthesis of Intermediate 1-8

A mixture of Intermediate 1-7 (330.00 mg, 460.35 μmol, 1 eq) in HCl/dioxane (5 mL, 2M) was stirred at 20° C. for 1 hr. The mixture was concentrated to give Intermediate 1-8 (300 mg, 445.51 μmol, 96.78% yield, 97% purity, HCl salt) as a white solid. LCMS: Retention time: 0.368 min, [M+H]⁺=617.3. SFC: Rt=1.436 min, ee value=97.4%

### Step 6: Synthesis of I-990

To a mixture of Intermediate 1-9 (118.19 mg, 459.29 μmol, 1 eq) in DMF (3 mL) was added EDCI (264.14 mg, 1.38 mmol, 3 eq), HOAt (62.51 mg, 459.29 μmol, 64.25 μL, 1 eq), NMM (371.64 mg, 3.67 mmol, 403.96 μL, 8 eq) and Intermediate 1-8 (300 mg, 459.29 μmol, 1 eq, HCl salt). The mixture was stirred at 20° C. for 2 hrs. The reaction mixture was poured into water (10 mL), then extracted with EA (10 mL*3), the organic layer was washed with brine (10 mL), dried over Na₂SO₄, filtered and concentrated to get the crude product. The crude product was purified by Prep-HPLC (column: Phenomenex luna C18 150*25 mm*10 um; mobile phase: [water (FA)-ACN]; gradient: 28%-588% B over 11 min), and the eluent was concentrated to remove organic solvents. The residual aqueous solution was lyophilized to give I-990 (189.12 mg, 219.29 μmol, 47.75% yield, 99.26% purity, FA salt) as a white solid. LCMS: Retention time: 15.554 min, [M+H]⁺=856.4. ¹HNMR (400 MHz, DMSO-d6) δ=8.25 (m, 1H), 8.16 (s, 1H), 7.98-7.92 (m, 1H), 7.91-7.79 (m, 2H), 7.46-7.39 (m, 1H), 7.35 (m, 1H), 7.22 (t, J=8.9 Hz, 1H), 6.78-6.61 (m, 1H), 4.32 (s, 2H), 4.21 (br s, 1H), 3.97 (m, 1H), 3.82-3.69 (m, 2H), 3.68-3.62 (m, 2H), 3.52-3.32 (m, 6H), 3.27-3.07 (m, 4H), 2.94 (m, 1H), 2.78-2.62 (m, 2H), 2.30-2.12 (m, 2H), 2.11-1.97 (m, 2H), 1.84 (br s, 2H), 1.73-1.48 (m, 9H), 1.45-1.28 (m, 11H), 1.26-1.19 (m, 1H), 1.17-1.03 (m, 3H), 1.01-0.79 (m, 2H). ¹⁹F NMR (377 MHz, DMSO-d6) δ=−119.72 (br s, 1F). SFC: Rt=1.505 min, de value >99%

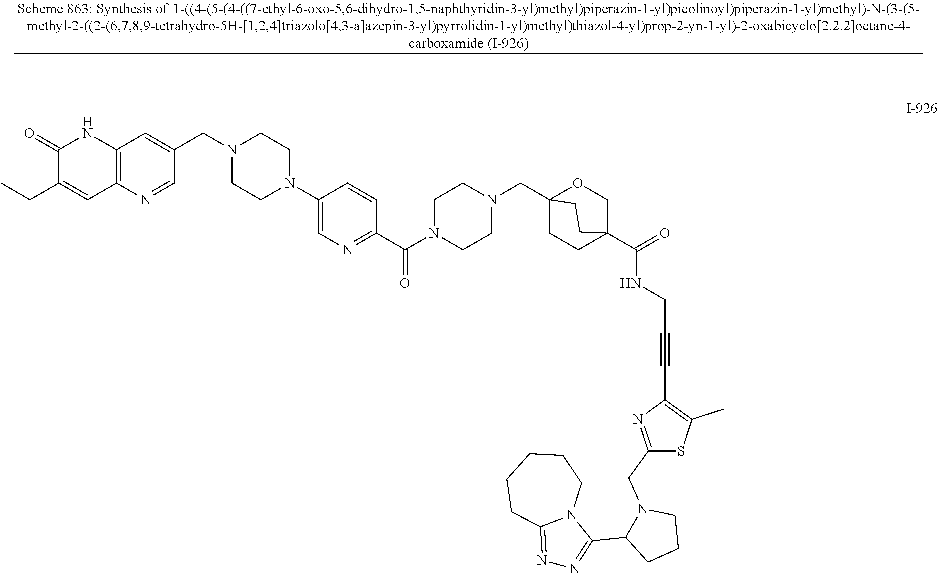 [SMILES: CCc1cc2ncc(CN3CCN(c4ccc(C(=O)N5CCN(CC67CCC(C(=O)NCC#Cc8nc(CN9CCCC9c9nnc%10n9CCCCC%10)sc8C)(CC6)CO7)CC5)nc4)CC3)cc2[nH]c1=O]

LCMS: Rt=0.375 min, [M+H]⁺=982.6. SFC: Rt: 4.345 min, 5.384 min. ¹H NMR (400 MHz, METHANOL-d4) δ=8.66-8.61 (m, 1H), 8.33 (br d, J=2.0 Hz, 1H), 7.98-7.94 (m, 1H), 7.86 (s, 1H), 7.70 (d, J=8.8 Hz, 1H), 7.53 (dd, J=2.6, 8.8 Hz, 1H), 4.61 (s, 2H), 4.50 (br dd, J=4.8, 9.4 Hz, 1H), 4.38-4.26 (m, 3H), 4.23-4.08 (m, 5H), 4.00 (s, 2H), 3.68 (br s, 5H), 3.53 (br s, 6H), 3.44-3.35 (m, 2H), 3.28 (s, 2H), 3.13-3.05 (m, 2H), 2.80 (q, J=8.2 Hz, 1H), 2.68 (q, J=7.4 Hz, 2H), 2.50-2.42 (m, 4H), 2.12-1.72 (m, 19H), 1.32-1.23 (m, 3H)

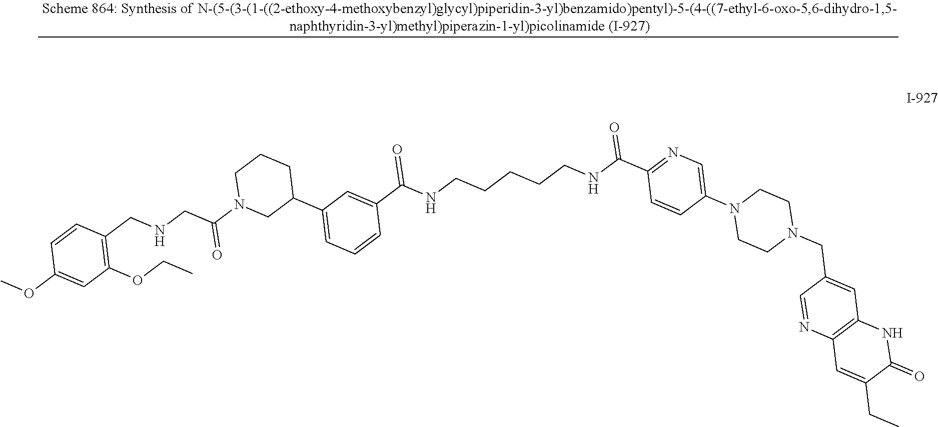 [SMILES: CCOc1cc(OC)ccc1CNCC(=O)N1CCCC(c2cccc(C(=O)NCCCCCNC(=O)c3ccc(N4CCN(Cc5cnc6cc(CC)c(=O)[nH]c6c5)CC4)cn3)c2)C1]

LCMS (Method D): Rt=0.349 min, [M+H]⁺=886.4. SFC: Rt: 1.581 min, 2.005 min. ¹H NMR (400 MHz, METHANOL-d₄) δ=8.49 (s, 1H), 8.22 (d, J=2.8 Hz, 1H), 7.90-7.82 (m, 2H), 7.77 (s, 1H), 7.72-7.62 (m, 2H), 7.46-7.31 (m, 3H), 7.15-7.09 (m, 1H), 6.53-6.41 (m, 2H), 4.56-4.49 (m, 1H), 4.09-3.99 (m, 2H), 3.86-3.80 (m, 1H), 3.78-3.70 (m, 7H), 3.50-3.43 (m, 3H), 3.42-3.37 (m, 8H), 3.15-3.03 (m, 1H), 2.74-2.63 (m, 8H), 2.04-1.97 (m, 1H), 1.86-1.77 (m, 2H), 1.71-1.64 (m, 4H), 1.52-1.46 (m, 2H), 1.45-1.41 (m, 2H), 1.40-1.37 (m, 1H), 1.29 (t, J=7.2 Hz, 3H)

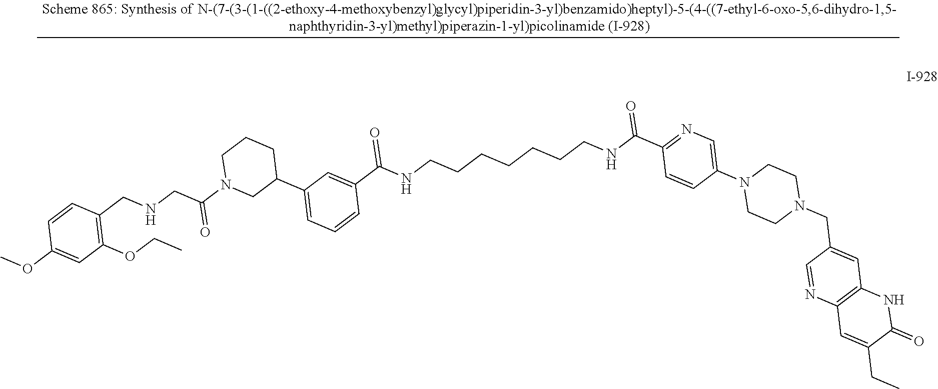 [SMILES: CCOc1cc(OC)ccc1CNCC(=O)N1CCCC(c2cccc(C(=O)NCCCCCCCNC(=O)c3ccc(N4CCN(Cc5cnc6cc(CC)c(=O)[nH]c6c5)CC4)cn3)c2)C1]

LCMS: 0.372 min, [M+H]⁺=914.5. SFC: 3.614 min, 5.413 min. ¹H NMR (400 MHz, METHANOL-d4) δ=8.53 (s, 1H), 8.49 (d, J=1.6 Hz, 1H), 8.26 (s, 1H), 7.89 (d, J=8.8 Hz, 1H), 7.83 (s, 1H), 7.78-7.64 (m, 3H), 7.48-7.33 (m, 3H), 7.23 (t, J=8.8 Hz, 1H), 6.65-6.44 (m, 2H), 4.53 (t, J=11.2 Hz, 1H), 4.18-4.00 (m, 4H), 3.87 (d, J=6.8 Hz, 1H), 3.81-3.77 (m, 3H), 3.76 (s, 3H), 3.38 (t, J=6.4 Hz, 7H), 3.22-3.12 (m, 3H), 2.84-2.59 (m, 8H), 2.09-1.99 (m, 1H), 1.90-1.75 (m, 4H), 1.73-1.68 (m, 1H), 1.62 (s, 4H), 1.48-1.39 (m, 8H), 1.28 (t, J=7.6 Hz, 3H)

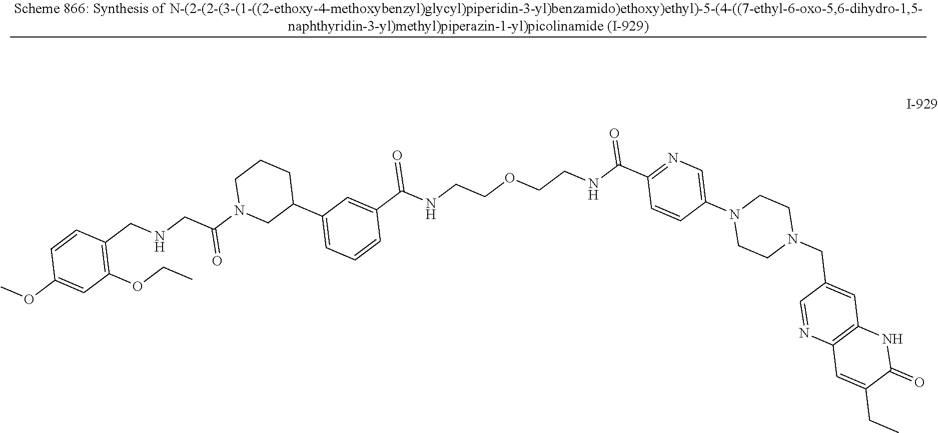 [SMILES: CCOc1cc(OC)ccc1CNCC(=O)N1CCCC(c2cccc(C(=O)NCCOCCNC(=O)c3ccc(N4CCN(Cc5cnc6cc(CC)c(=O)[nH]c6c5)CC4)cn3)c2)C1]

LCMS (Method D): Rt: 0.336 min, [M+H]⁺=888.5. SFC: Rt: 1.211 min, 1.389 min. ¹H NMR (400 MHz, METHANOL-d4) δ=8.49 (s, 1H), 8.04-8.01 (m, 1H), 7.86-7.75 (m, 3H), 7.73-7.62 (m, 2H), 7.43-7.28 (m, 3H), 7.10-7.07 (m, 1H), 6.51-6.38 (m, 2H), 4.54-4.47 (m, 1H), 4.02-3.98 (m, 2H), 3.84-3.78 (m, 1H), 3.76-3.72 (m, 6H), 3.71-3.65 (m, 5H), 3.60 (s, 4H), 3.49-3.41 (m, 2H), 3.36-3.33 (m, 4H), 3.11-3.01 (m, 1H), 2.75-2.67 (m, 4H), 2.67-2.63 (m, 4H), 2.04-1.96 (m, 1H), 1.85-1.75 (m, 2H), 1.60-1.49 (m, 1H), 1.40-1.36 (m, 3H), 1.28 (t, J=7.2 Hz, 3H).

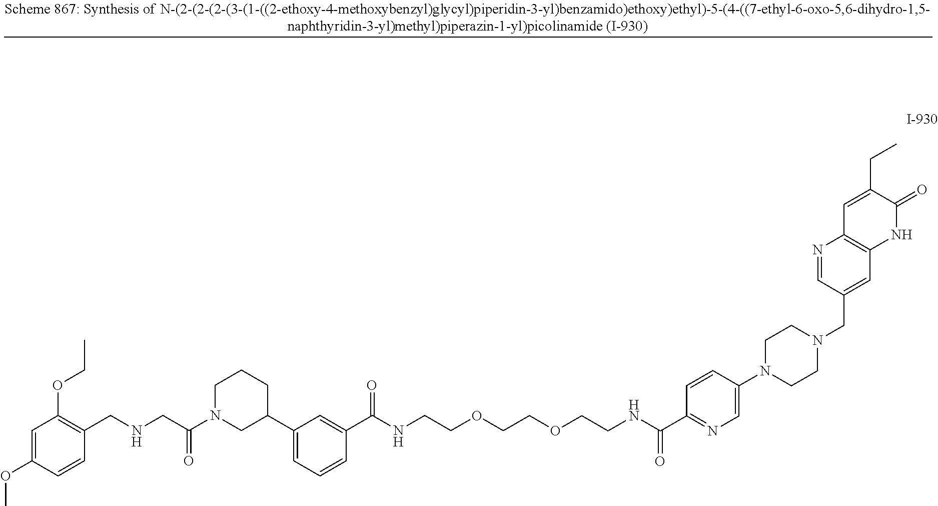 [SMILES: CCOc1cc(OC)ccc1CNCC(=O)N1CCCC(c2cccc(C(=O)NCCOCCOCCNC(=O)c3ccc(N4CCN(Cc5cnc6cc(CC)c(=O)[nH]c6c5)CC4)cn3)c2)C1]

LCMS: Rt: 0.336 min, [M+H]⁺=932.5. SFC: Rt: 1.039 min, 1.269 min. ¹H NMR (400 MHz, METHANOL-d4) δ=8.48 (s, 1H), 8.21-8.20 (m, 1H), 7.91-7.79 (m, 2H), 7.77-7.63 (m, 3H), 7.40-7.30 (m, 3H), 7.15-7.10 (m, 1H), 6.51-6.41 (m, 2H), 4.64-4.45 (m, 3H), 4.04-3.99 (m, 2H), 3.83-3.77 (m, 2H), 3.77-3.74 (m, 4H), 3.71 (s, 2H), 3.70-3.66 (m, 7H), 3.66-3.64 (m, 1H), 3.60-3.56 (m, 3H), 3.55-3.46 (m, 3H), 3.46-3.39 (m, 1H), 3.35 (d, J=4.0 Hz, 4H), 3.17-2.96 (m, 2H), 2.72-2.67 (m, 2H), 2.67-2.60 (m, 6H), 1.97 (d, J=11.2 Hz, 1H), 1.82-1.73 (m, 2H), 1.61-1.50 (m, 1H), 1.44-1.37 (m, 3H), 1.29 (t, J=7.2 Hz, 3H).

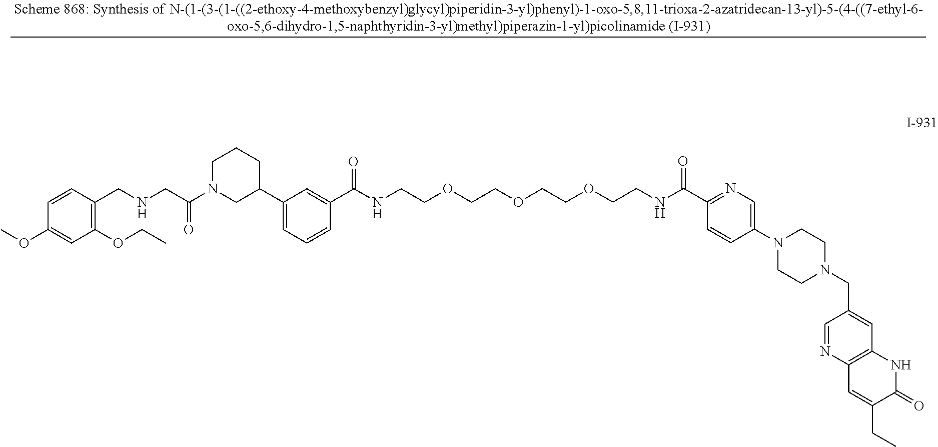 [SMILES: CCOc1cc(OC)ccc1CNCC(=O)N1CCCC(c2cccc(C(=O)NCCOCCOCCOCCNC(=O)c3ccc(N4CCN(Cc5cnc6cc(CC)c(=O)[nH]c6c5)CC4)cn3)c2)C1]

LCMS: Rt: 0.333 min, [M+H]⁺=976.6. SFC: Rt: 1.042 min, 1.246 min. ¹H NMR (400 MHz, METHANOL-d4) δ=8.47 (s, 1H), 8.23 (s, 1H), 7.92-7.79 (m, 2H), 7.76-7.64 (m, 3H), 7.41-7.30 (m, 3H), 7.12-7.08 (m, 1H), 6.51-6.40 (m, 2H), 4.64-4.47 (m, 2H), 4.07-3.96 (m, 2H), 3.83-3.77 (m, 1H), 3.75 (d, J=5.6 Hz, 4H), 3.73-3.69 (m, 3H), 3.66-3.62 (m, 7H), 3.62-3.58 (m, 5H), 3.57-3.51 (m, 4H), 3.50-3.41 (m, 2H), 3.39-3.33 (m, 4H), 3.12-3.01 (m, 1H), 2.72-2.66 (m, 2H), 2.65-2.61 (m, 5H), 2.04-1.95 (m, 1H), 1.83-1.71 (m, 2H), 1.61-1.48 (m, 1H), 1.44-1.36 (m, 3H), 1.28 (t, J=7.2 Hz, 3H).

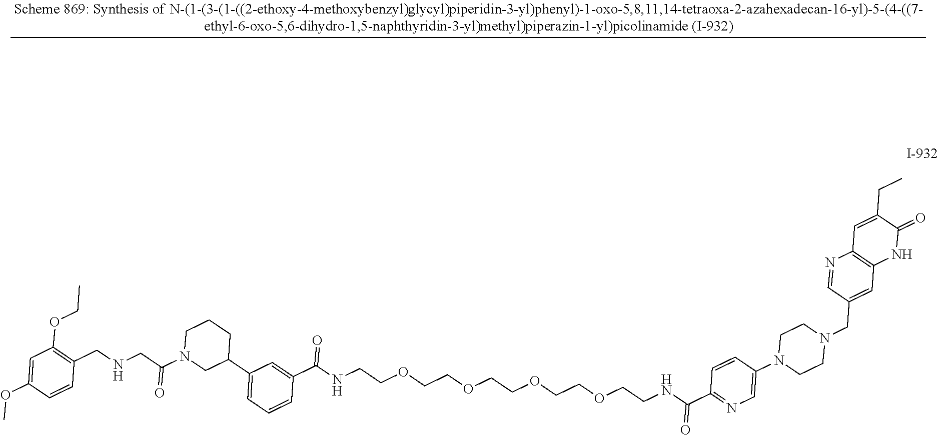 [SMILES: CCOc1cc(OC)ccc1CNCC(=O)N1CCCC(c2cccc(C(=O)NCCOCCOCCOCCOCCNC(=O)c3ccc(N4CCN(Cc5cnc6cc(CC)c(=O)[nH]c6c5)CC4)cn3)c2)C1]

LCMS (Method D): Rt: 0.350 min, [M+H]⁺=1020.7. SFC: Rt: 0.627 min, 0.715 min. ¹H NMR (400 MHz, METHANOL-d4) δ=8.48 (d, J=1.2 Hz, 1H), 8.25 (d, J=2.8 Hz, 1H), 7.89 (d, J=8.8 Hz, 1H), 7.83 (s, 1H), 7.76 (s, 1H), 7.74-7.65 (m, 2H), 7.44-7.30 (m, 3H), 7.17-7.07 (m, 1H), 6.52-6.39 (m, 2H), 4.52 (d, J=11.6 Hz, 1H), 4.08-3.98 (m, 2H), 3.85-3.79 (m, 1H), 3.75 (d, J=5.2 Hz, 3H), 3.74-3.68 (m, 4H), 3.64 (d, J=4.8 Hz, 2H), 3.61 (d, J=2.4 Hz, 6H), 3.59 (s, 8H), 3.56 (d, J=5.2 Hz, 4H), 3.50-3.41 (m, 2H), 3.37 (s, 4H), 3.14-2.99 (m, 1H), 2.76-2.54 (m, 8H), 1.99 (d, J=12.0 Hz, 1H), 1.87-1.71 (m, 2H), 1.64-1.48 (m, 1H), 1.45-1.35 (m, 3H), 1.32-1.24 (m, 3H).

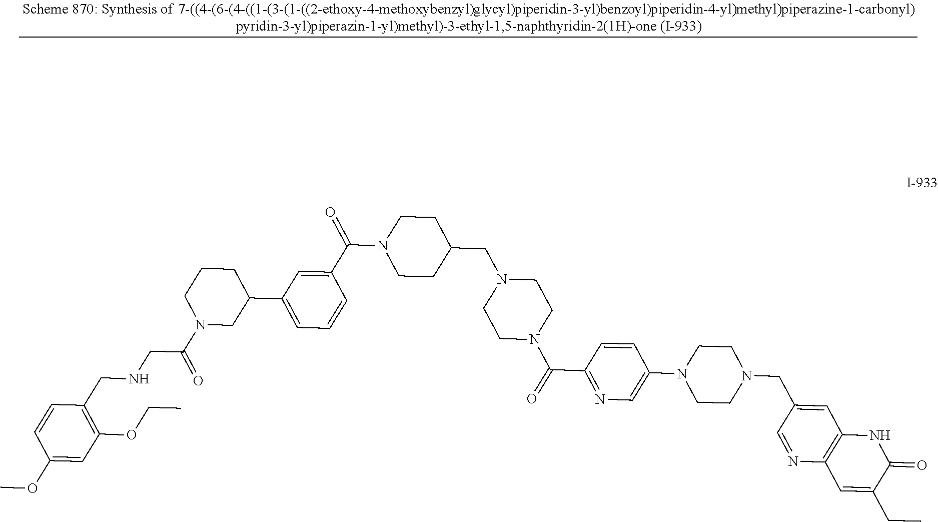 [SMILES: CCOc1cc(OC)ccc1CNCC(=O)N1CCCC(c2cccc(C(=O)N3CCC(CN4CCN(C(=O)c5ccc(N6CCN(Cc7cnc8cc(CC)c(=O)[nH]c8c7)CC6)cn5)CC4)CC3)c2)C1]

LCMS: Rt: 0.310 min, [M+H]⁺=967.6. SFC: Rt: 1.914 min. H NMR (400 MHz, METHANOL-d4) δ=8.49 (d, J=1.6 Hz, 1H), 8.23 (d, J=2.8 Hz, 1H), 7.86-7.74 (m, 2H), 7.52-7.47 (m, 1H), 7.44-7.34 (m, 3H), 7.30-7.23 (m, 2H), 7.17-7.07 (m, 1H), 6.53-6.49 (m, 1H), 6.48-6.42 (m, 1H), 4.67-4.57 (m, 1H), 4.55-4.46 (m, 1H), 4.10-3.99 (m, 2H), 3.86-3.79 (m, 1H), 3.76 (d, J=2.4 Hz, 4H), 3.75-3.68 (m, 6H), 3.60 (s, 2H), 3.52-3.41 (m, 2H), 3.40-3.34 (m, 4H), 3.16-3.01 (m, 2H), 2.92-2.80 (m, 1H), 2.74-2.60 (m, 8H), 2.56-2.37 (m, 4H), 2.27 (d, J=6.4 Hz, 2H), 2.01 (d, J=11.6 Hz, 1H), 1.95-1.86 (m, 2H), 1.85-1.70 (m, 3H), 1.66-1.49 (m, 1H), 1.47-1.35 (m, 3H), 1.32-1.25 (m, 3H), 1.24-1.08 (m, 2H)

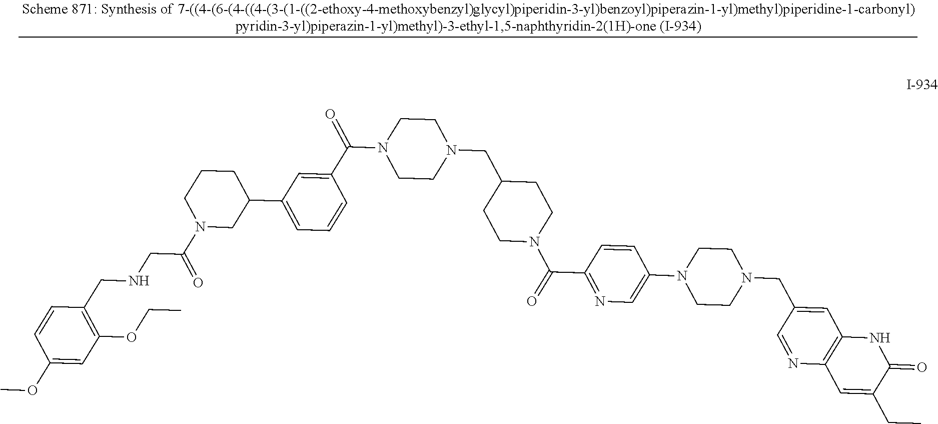 [SMILES: CCOc1cc(OC)ccc1CNCC(=O)N1CCCC(c2cccc(C(=O)N3CCN(CC4CCN(C(=O)c5ccc(N6CCN(Cc7cnc8cc(CC)c(=O)[nH]c8c7)CC6)cn5)CC4)CC3)c2)C1]

LCMS (Method D): Rt=0.301 min, [M+H]⁺=967.7. SFC (Method AC): Rt: 6.098 min, 6.683 min. ¹H NMR (400 MHz, METHANOL-d₄) δ=8.49 (s, 1H), 8.23 (d, J=2.4 Hz, 1H), 7.83 (s, 1H), 7.77 (s, 1H), 7.49-7.35 (m, 4H), 7.34-7.23 (m, 2H), 7.13 (t, J=8.0 Hz, 1H), 6.51 (d, J=4.0 Hz, 1H), 6.48-6.43 (m, 1H), 4.64-4.46 (m, 2H), 4.10-4.01 (m, 2H), 3.95-3.87 (m, 1H), 3.85-3.68 (m, 10H), 3.51-3.35 (m, 8H), 3.16-3.03 (m, 2H), 2.91-2.80 (m, 1H), 2.76-2.62 (m, 8H), 2.57-2.36 (m, 4H), 2.27 (d, J=6.4 Hz, 2H), 2.01 (d, J=11.6 Hz, 1H), 1.96-1.72 (m, 5H), 1.65-1.51 (m, 1H), 1.47-1.38 (m, 3H), 1.29 (t, J=7.2 Hz, 3H), 1.25-1.13 (m, 2H)

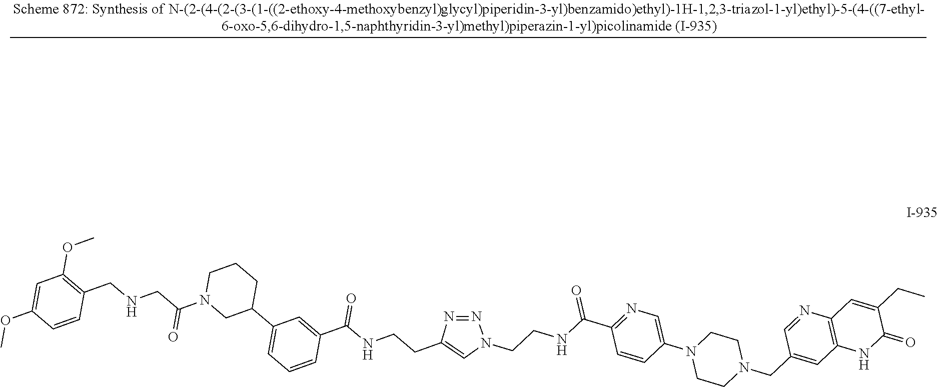 [SMILES: CCc1cc2ncc(CN3CCN(c4ccc(C(=O)NCCn5cc(CCNC(=O)c6cccc(C7CCCN(C(=O)CNCc8ccc(OC)cc8OC)C7)c6)nn5)nc4)CC3)cc2[nH]c1=O]

LCMS: Rt=0.651 min, [M+H]⁺=939.5. SFC: Rt=1.567 min, 1.847 min. H NMR (400 MHz, METHANOL-d4) δ=8.48 (s, 1H), 8.22 (s, 1H), 7.86-7.61 (m, 6H), 7.43-7.34 (m, 2H), 7.33-7.26 (m, 1H), 7.15-7.07 (m, 1H), 6.54-6.38 (m, 2H), 4.61-4.56 (m, 2H), 4.54-4.47 (m, 1H), 4.08-3.98 (m, 2H), 3.85-3.81 (m, 2H), 3.81-3.66 (m, 8H), 3.65-3.58 (m, 2H), 3.52-3.41 (m, 2H), 3.40-3.35 (m, 4H), 3.15-3.04 (m, 1H), 3.01-2.96 (m, 2H), 2.74-2.60 (m, 8H), 2.03-1.94 (m, 1H), 1.86-1.73 (m, 2H), 1.61-1.48 (m, 1H), 1.46-1.36 (m, 3H), 1.31-1.24 (m, 3H)

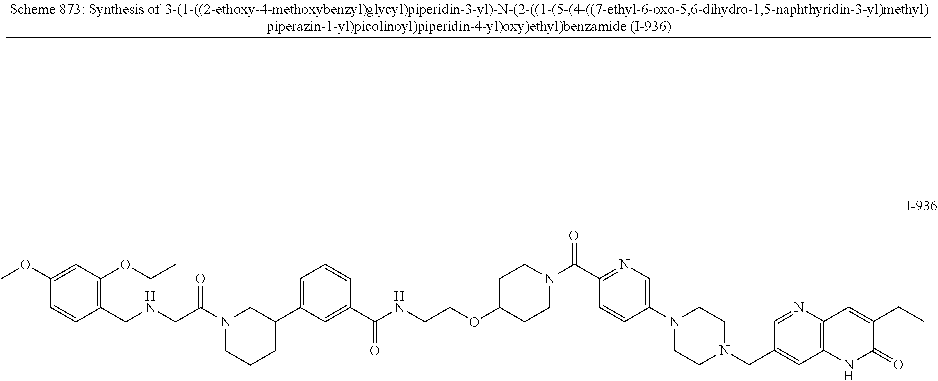 [SMILES: CCOc1cc(OC)ccc1CNCC(=O)N1CCCC(c2cccc(C(=O)NCCOC3CCN(C(=O)c4ccc(N5CCN(Cc6cnc7cc(CC)c(=O)[nH]c7c6)CC5)cn4)CC3)c2)C1]

LCMS: Rt=0.330 min, [M+H]⁺=928.6. SFC: Rt=1.512 min, 1.985 min. H NMR (400 MHz, METHANOL-d4) δ=8.51 (d, J=1.2 Hz, 1H), 8.23 (s, 1H), 7.85 (s, 1H), 7.78 (s, 1H), 7.75-7.67 (m, 2H), 7.49-7.39 (m, 4H), 7.17-7.10 (m, 1H), 6.54-6.43 (m, 2H), 4.54 (d, J=11.2 Hz, 1H), 4.11-3.95 (m, 3H), 3.80-3.74 (m, 7H), 3.73-3.68 (m, 4H), 3.59 (d, J=5.6 Hz, 3H), 3.54-3.47 (m, 2H), 3.38 (s, 5H), 3.17-3.06 (m, 1H), 2.76-2.65 (m, 8H), 2.07-1.94 (m, 2H), 1.92-1.79 (m, 3H), 1.75-1.57 (m, 3H), 1.46-1.39 (m, 4H), 1.30 (t, J=7.6 Hz, 3H)1

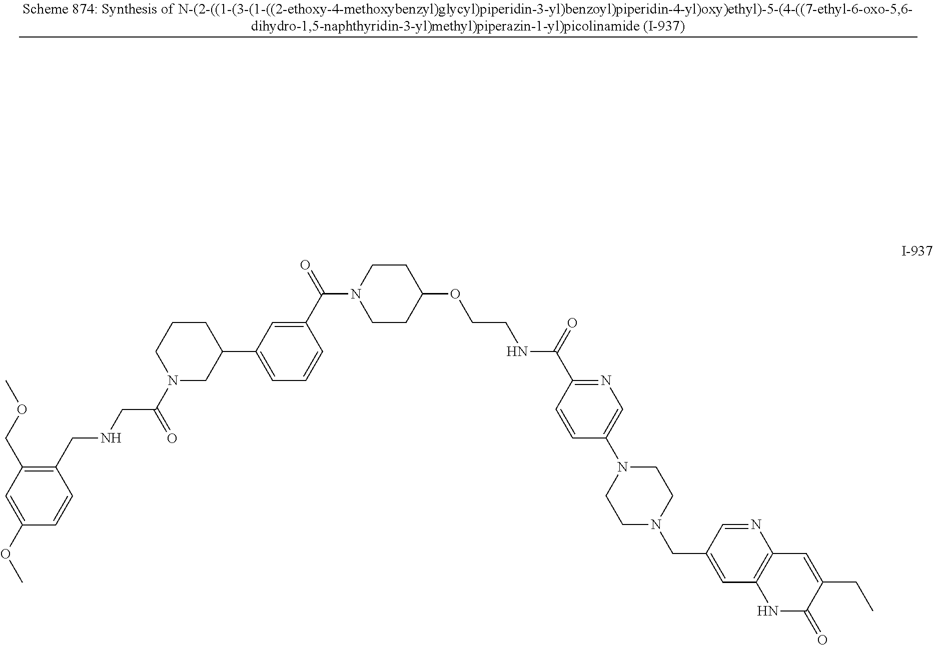 [SMILES: CCc1cc2ncc(CN3CCN(c4ccc(C(=O)NCCOC5CCN(C(=O)c6cccc(C7CCCN(C(=O)CNCc8ccc(OC)cc8COC)C7)c6)CC5)nc4)CC3)cc2[nH]c1=O]

LCMS: Rt=0.693 min, [M+H]⁺=928.6. SFC: Rt=0.713 min, 1.153 min. ¹H NMR (400 MHz, METHANOL-d4) δ=8.49 (s, 1H), 8.33-8.22 (m, 1H), 7.91 (d, J=8.8 Hz, 1H), 7.83 (s, 1H), 7.76 (s, 1H), 7.44-7.34 (m, 3H), 7.30-7.20 (m, 2H), 7.13-7.09 (m, 1H), 6.49 (d, J=4.0 Hz, 1H), 6.46-4.42 (m, 1H), 4.59-4.42 (m, 1H), 4.09-3.92 (m, 3H), 3.85-3.66 (m, 11H), 3.62-3.52 (m, 4H), 3.52-3.43 (m, 2H), 3.39 (s, 4H), 3.26-3.24 (m, 1H), 3.15-2.98 (m, 1H), 2.75-2.60 (m, 8H), 2.05-1.93 (m, 2H), 1.85-1.78 (m, 2H), 1.77-1.51 (m, 4H), 1.45-1.36 (m, 3H), 1.30-1.26 (m, 3H)

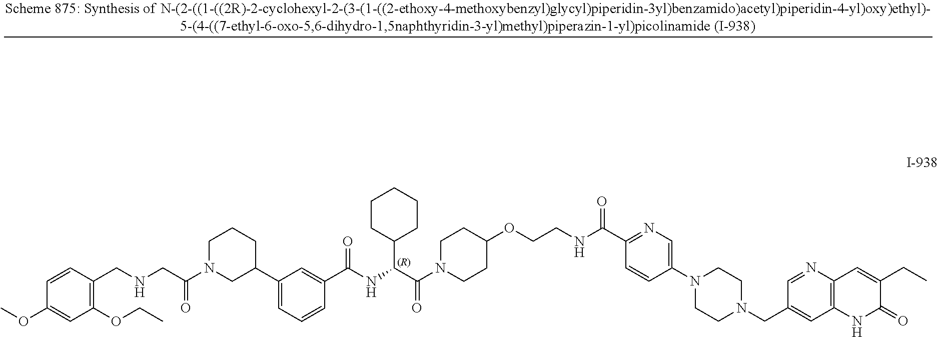

LCMS: Rt: 0.763 min, [M+H]⁺=1067.4. SFC: Rt: 1.599 min, 1.917 min. ¹H NMR (4 400 MHz, METHANOL-d4) δ=8.50-8.46 (m, 1H), 8.30-8.24 (m, 1H), 7.94-7.87 (m, 1H), 7.83 (s, 1H), 7.78-7.64 (m, 3H), 7.47-7.30 (m, 3H), 7.17-7.07 (m, 1H), 6.53-6.40 (m, 2H), 4.95-4.89 (m, 2H), 4.55-4.46 (m, 1H), 4.09-3.79 (m, 4H), 3.78-3.71 (m, 6H), 3.70-3.63 (m, 4H), 3.63-3.43 (m, 5H), 3.43-3.32 (m, 5H), 3.16-3.00 (m, 1H), 2.80-2.52 (m, 8H), 2.02-1.50 (m, 14H), 1.46-1.35 (m, 3H), 1.34-1.16 (m, 6H), 1.16-1.02 (m, 2H)

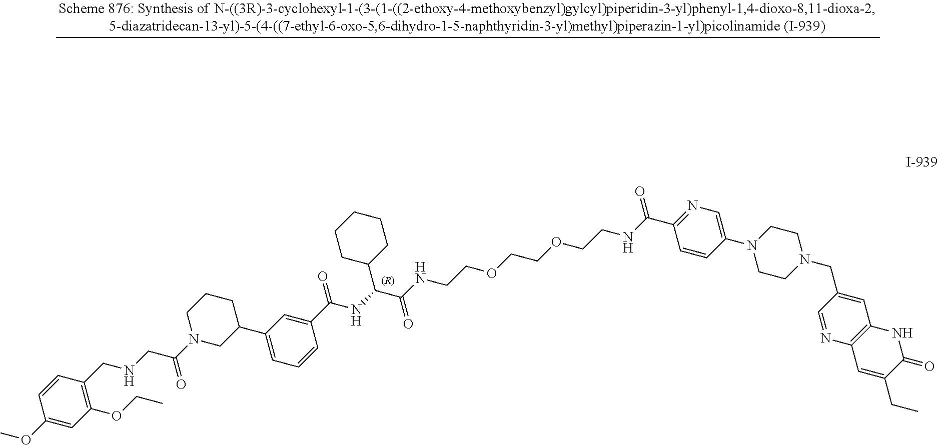

LCMS: Rt=0.362 min, [M+H]⁺=1071.6

SFC: Rt=1.039 min, 1.187 min. ¹H NMR (400 MHz, METHANOL-d4) δ=8.47 (s, 1H), 8.27 (s, 1H), 7.95-7.79 (m, 2H), 7.76-7.66 (m, 3H), 7.42-7.31 (m, 3H), 7.14-7.09 (m, 1H), 6.52-6.40 (m, 2H), 4.54-4.47 (m, 1H), 4.40-4.34 (m, 1H), 4.08-3.99 (m, 2H), 3.84-3.74 (m, 5H), 3.72-3.68 (m, 3H), 3.66-3.60 (m, 6H), 3.57 (d, J=4.4 Hz, 4H), 3.50-3.40 (m, 3H), 3.36 (d, J=4.4 Hz, 5H), 3.13-3.01 (m, 1H), 2.72-2.57 (m, 8H), 1.97 (d, J=12.4 Hz, 1H), 1.87-1.58 (m, 9H), 1.45-1.36 (m, 3H), 1.28 (t, J=7.6 Hz, 4H), 1.24-1.01 (m, 4H)

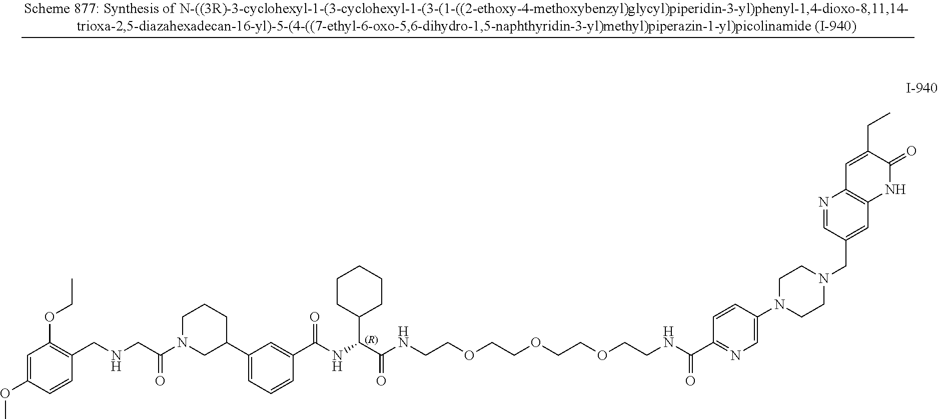

LCMS: Rt: 0.747 min, [M+H]⁺=1115.6. SFC: Rt: 1.120 min, 1.271 min. ¹H NMR (400 MHz, METHANOL-d4) δ=8.56-8.41 (m, 1H), 8.26 (s, 1H), 7.90 (d, J=8.8 Hz, 1H), 7.83 (s, 1H), 7.78-7.66 (m, 3H), 7.47-7.31 (m, 3H), 7.18-7.06 (m, 1H), 6.60-6.34 (m, 2H), 4.47-4.33 (m, 1H), 4.08-3.99 (m, 2H), 3.79-3.68 (m, 8H), 3.67-3.49 (m, 17H), 3.42-3.34 (m, 7H), 2.75-2.57 (m, 7H), 2.03-1.93 (m, 1H), 1.86-1.65 (m, 9H), 1.45-1.42 (m, 2H), 1.41-1.36 (m, 2H), 1.31-1.21 (m, 6H), 1.17-1.06 (m, 2H)

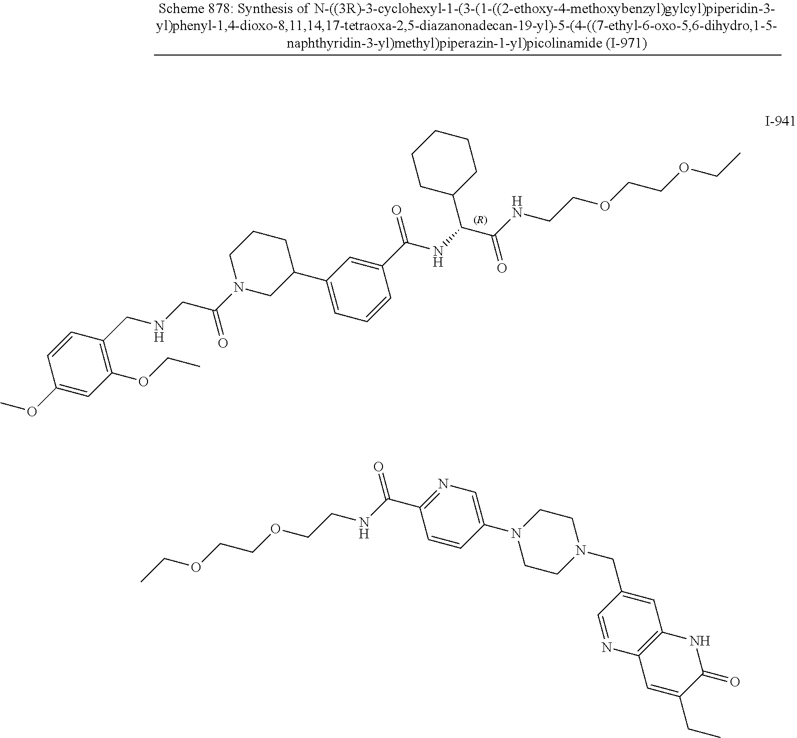

LCMS: Rt: 0.754 min, [M+H]⁺=1159.7. SFC: Rt: 1.133 min, 1.306 min. ¹H NMR (400 MHz, METHANOL-d4) δ=8.48 (s, 1H), 8.27 (d, J=2.8 Hz, 1H), 7.91 (d, J=8.8 Hz, 1H), 7.82 (s, 1H), 7.78-7.63 (m, 3H), 7.52-7.33 (m, 3H), 7.18-7.06 (m, 1H), 6.58-6.35 (m, 2H), 4.56-4.47 (m, 1H), 4.40 (brt, J=7.6 Hz, 1H), 4.09-3.98 (m, 2H), 3.78-3.69 (m, 7H), 3.68-3.61 (m, 9H), 3.60-3.50 (m, 10H), 3.49-3.42 (m, 3H), 3.38 (br s, 6H), 3.14-3.01 (m, 1H), 2.73-2.61 (m, 7H), 2.07-1.94 (m, 1H), 1.89-1.65 (m, 9H), 1.45-1.37 (m, 3H), 1.33-1.20 (m, 6H), 1.17-1.06 (m, 2H)

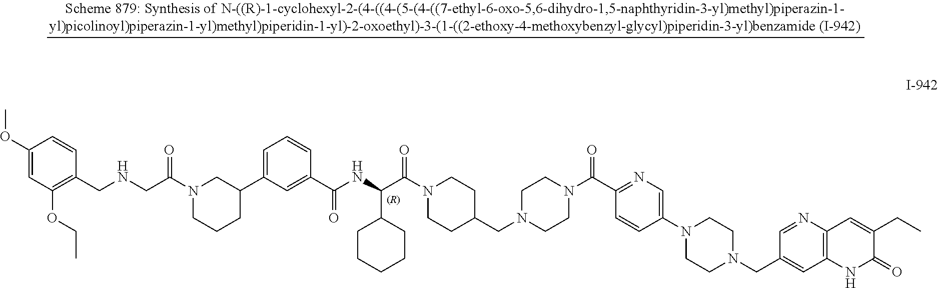

LCMS: Rt: 0.747 min, [M+H]⁺=1106.5. SFC: Rt: 1.931 min, 2.347 min. ¹H NMR (4 400 MHz, METHANOL-d4) δ=8.51-8.44 (m, 1H), 8.26-8.19 (m, 1H), 7.83 (s, 1H), 7.78-7.66 (m, 3H), 7.54-7.35 (m, 4H), 7.17-7.08 (m, 1H), 6.54-6.40 (m, 2H), 4.99-4.91 (m, 1H), 4.59-4.47 (m, 2H), 4.36-4.25 (m, 1H), 4.11-3.98 (m, 2H), 3.82-3.68 (m, 9H), 3.66-3.53 (m, 2H), 3.52-3.33 (m, 6H), 3.30-3.26 (m, 1H), 3.23-3.03 (m, 2H), 2.82-2.59 (m, 9H), 2.57-2.35 (m, 4H), 2.30-2.17 (m, 2H), 2.04-1.51 (m, 13H), 1.47-1.36 (m, 3H), 1.35-1.18 (m, 7H), 1.17-1.03 (m, 3H)

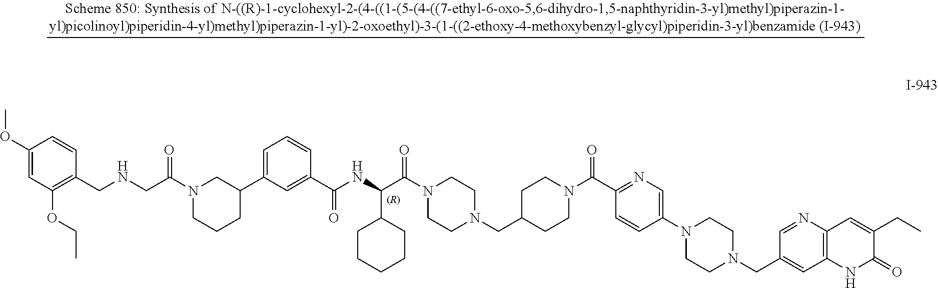

LCMS: Rt=0.356 min, [M+H]⁺=1106.7. SFC: Rt=0.954 min, 1.145 min. ¹H NMR (400 MHz, METHANOL-d4) δ=8.50 (d, J=1.6 Hz, 1H), 8.23 (d, J=2.0 Hz, 1H), 7.84 (s, 1H), 7.78-7.69 (m, 3H), 7.48-7.41 (m, 4H), 7.13-7.10 (m, 1H), 6.53-6.44 (m, 2H), 4.61-4.53 (m, 2H), 4.09-4.02 (m, 2H), 3.94-3.88 (m, 1H), 3.86-3.80 (m, 2H), 3.76 (d, J=3.2 Hz, 4H), 3.75-3.72 (m, 4H), 3.57-3.55 (m, 1H), 3.51-3.45 (m, 2H), 3.40-3.36 (m, 4H), 3.19-3.03 (m, 3H), 2.91-2.84 (m, 1H), 2.80-2.71 (m, 2H), 2.70-2.66 (m, 6H), 2.52-2.38 (m, 4H), 2.25 (d, J=7.2 Hz, 2H), 2.05-2.00 (m, 1H), 1.94-1.83 (m, 6H), 1.82-1.74 (m, 4H), 1.72-1.67 (m, 2H), 1.42-1.39 (m, 4H), 1.29-1.26 (m, 6H), 1.24-1.19 (m, 2H), 1.11 (d, J=12.0 Hz, 2H)

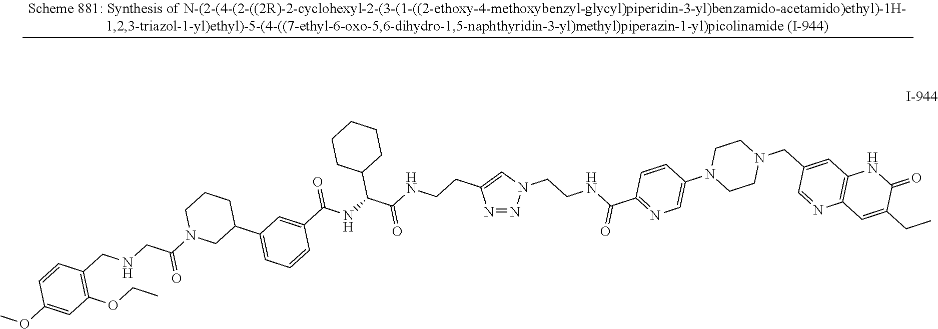 [SMILES: CCOc1cc(OC)ccc1CNCC(=O)N1CCCC(c2cccc(C(=O)N[C@@H](C(=O)NCCc3cn(CCNC(=O)c4ccc(N5CCN(Cc6cnc7cc(CC)c(=O)[nH]c7c6)CC5)cn4)nn3)C3CCCCC3)c2)C1]

LCMS: Rt: 0.372 min, [M+H]⁺=1078.4. SFC: Rt: 2.428 min, 5.324 min. ¹H NMR (400 MHz, METHANOL-d4) δ=8.48 (s, 1H), 8.22 (s, 1H), 7.86-7.77 (m, 3H), 7.74 (d, J=7.2 Hz, 2H), 7.69 (d, J=5.6 Hz, 1H), 7.43-7.27 (m, 3H), 7.17-7.07 (m, 1H), 6.53-6.40 (m, 2H), 4.64-4.44 (m, 4H), 4.35-4.23 (m, 1H), 4.09-3.97 (m, 2H), 3.86-3.77 (m, 3H), 3.75 (d, J=6.0 Hz, 4H), 3.71 (s, 2H), 3.53-3.33 (m, 8H), 3.15-3.02 (m, 1H), 2.94-2.85 (m, 2H), 2.73-2.56 (m, 8H), 1.98 (d, J=11.2 Hz, 1H), 1.86-1.71 (m, 6H), 1.69-1.52 (m, 3H), 1.48-1.37 (m, 3H), 1.32-1.17 (m, 6H), 1.09-0.97 (m, 2H)

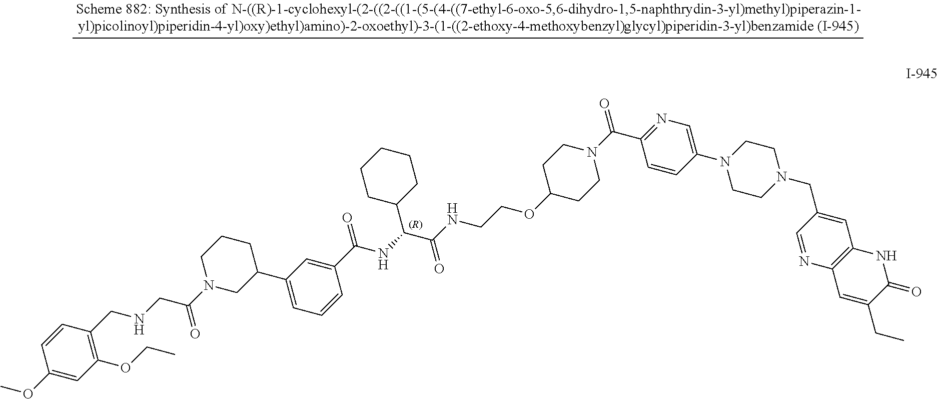

LCMS: Rt=0.728 min, [M+H]⁺=1067.7. SFC: Rt=0.659 min, 0.750 min. ¹H NMR (400 MHz, METHANOL-d4) δ=8.51 (s, 1H), 8.24 (s, 1H), 7.86 (s, 1H), 7.80-7.71 (m, 3H), 7.48-7.41 (m, 4H), 7.14-7.11 (m, 1H), 6.54-6.45 (m, 2H), 4.57-4.51 (m, 1H), 4.42-4.37 (m, 1H), 4.10-4.03 (m, 2H), 3.96-3.89 (m, 1H), 3.87-3.82 (m, 1H), 3.79-3.72 (m, 8H), 3.67-3.57 (m, 4H), 3.49 (d, J=6.4 Hz, 3H), 3.39 (s, 6H), 3.17-3.08 (m, 1H), 2.74-2.65 (m, 8H), 2.02 (d, J=12.8 Hz, 1H), 1.88 (d, J=8.4 Hz, 4H), 1.83-1.77 (m, 4H), 1.70-1.59 (m, 3H), 1.43-1.41 (m, 4H), 1.31-1.28 (m, 6H), 1.23 (d, J=11.6 Hz, 1H), 1.18-1.08 (m, 2H)

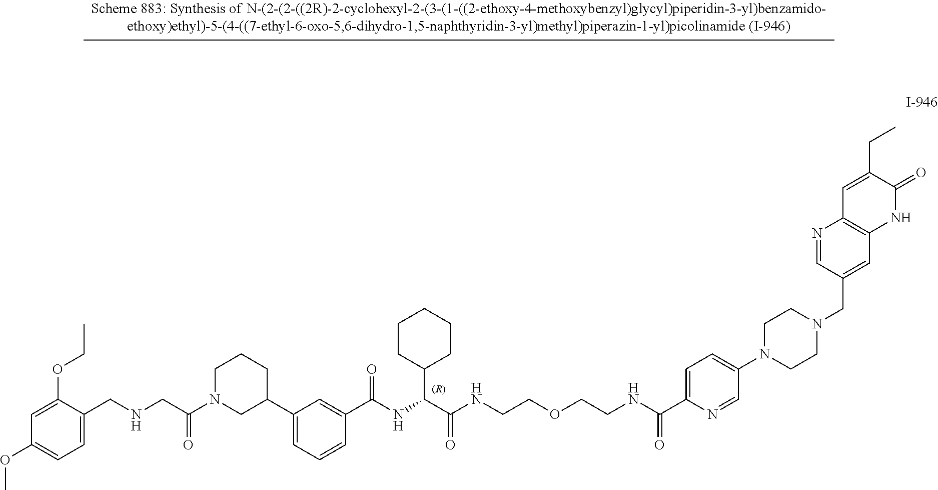

LCMS: Rt: 0.373 min, [M+H]⁺=1027.6. SFC: Rt: 1.016 min/1.189 min. ¹H NMR (400 MHz, METHANOL-d4) δ=8.47 (d, J=1.6 Hz, 1H), 8.31 (d, J=2.8 Hz, 1H), 7.96-7.80 (m, 2H), 7.78-7.65 (m, 3H), 7.44-7.33 (m, 3H), 7.11-7.08 (m, 1H), 6.56-6.37 (m, 2H), 4.54-4.45 (m, 1H), 4.38 (t, J=8.4 Hz, 1H), 4.03-3.98 (m, 2H), 3.85-3.78 (m, 1H), 3.76 (d, J=0.8 Hz, 2H), 3.75 (s, 2H), 3.72-3.69 (m, 3H), 3.65-3.58 (m, 4H), 3.57-3.52 (m, 2H), 3.50-3.44 (m, 2H), 3.39 (s, 5H), 3.35 (d, J=3.6 Hz, 1H), 3.13-3.02 (m, 1H), 2.73-2.67 (m, 2H), 2.65 (d, J=6.4 Hz, 6H), 2.03-1.95 (m, 1H), 1.81 (d, J=10.4 Hz, 3H), 1.77-1.71 (m, 1H), 1.71-1.62 (m, 3H), 1.61-1.46 (m, 2H), 1.41-1.36 (m, 3H), 1.28 (t, J=7.2 Hz, 3H), 1.25-1.13 (m, 2H), 1.12-0.93 (m, 3H).

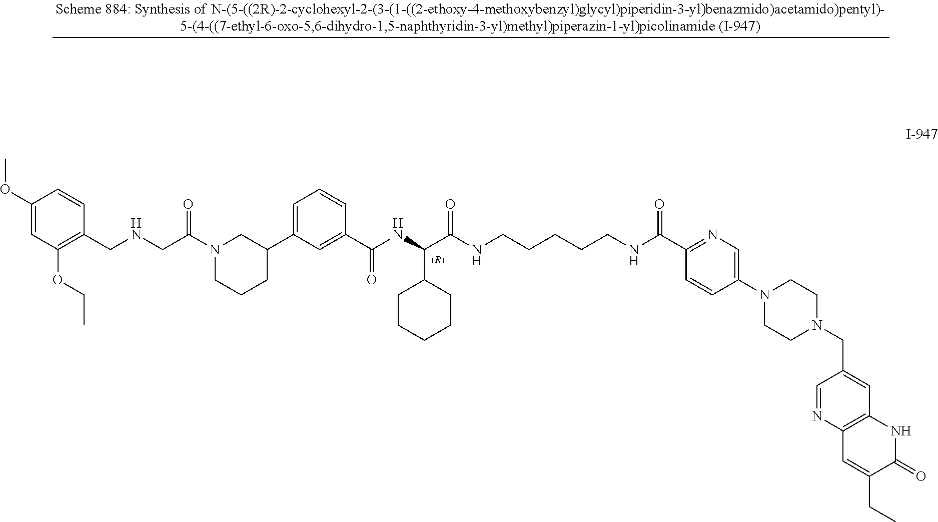

LCMS: Rt: 0.380 min, [M+H]⁺=1025.8. SFC: Rt: 1.187 min, 1.414 min. ¹H NMR (400 MHz, METHANOL-d4) δ=8.49 (d, J=1.6 Hz, 1H), 8.26 (d, J=2.4 Hz, 1H), 7.94-7.82 (m, 2H), 7.77-7.68 (m, 3H), 7.46-7.34 (m, 3H), 7.16-7.09 (m, 1H), 6.52-6.42 (m, 2H), 4.55-4.48 (m, 1H), 4.34-4.32 (m, 1H), 4.08-3.99 (m, 2H), 3.84-3.79 (m, 1H), 3.76 (d, J=4.8 Hz, 4H), 3.73 (s, 3H), 3.48 (d, J=6.4 Hz, 2H), 3.42-3.35 (m, 7H), 3.27-3.16 (m, 2H), 3.14-3.04 (m, 1H), 2.72-2.62 (m, 8H), 2.03-1.97 (m, 1H), 1.87-1.78 (m, 4H), 1.77-1.71 (m, 2H), 1.66-1.56 (m, 6H), 1.46-1.41 (m, 3H), 1.41-1.36 (m, 2H), 1.28 (t, J=7.2 Hz, 4H), 1.25-1.15 (m, 2H), 1.14-1.04 (m, 2H).

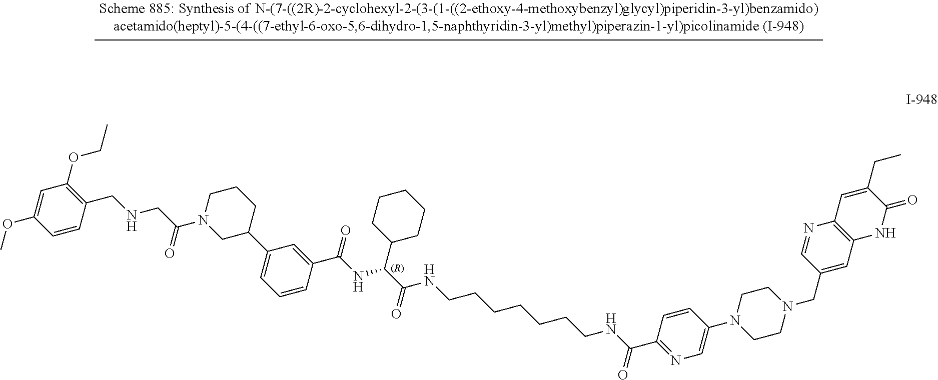

LCMS: Rt=0.402 min, [M+H]⁺=1053.8. SFC: Rt=1.391 min, 1.642 min. ¹H NMR (400 MHz, METHANOL-d4) δ=8.49 (d, J=1.6 Hz, 1H), 8.26 (d, J=2.8 Hz, 1H), 7.93-7.81 (m, 2H), 7.79-7.66 (m, 3H), 7.45-7.34 (m, 3H), 7.17-7.08 (m, 1H), 6.55-6.40 (m, 2H), 4.54-4.48 (m, 1H), 4.37-4.31 (m, 1H), 4.08-3.99 (m, 2H), 3.76 (d, J=5.2 Hz, 3H), 3.73 (s, 2H), 3.71-3.68 (m, 3H), 3.54-3.47 (m, 3H), 3.40-3.37 (m, 4H), 3.25-3.16 (m, 2H), 3.08 (d, J=6.0 Hz, 1H), 2.69-2.64 (m, 6H), 2.58 (t, J=6.0 Hz, 1H), 2.00 (d, J=12.8 Hz, 1H), 1.88-1.81 (m, 3H), 1.79-1.73 (m, 4H), 1.68-1.52 (m, 8H), 1.45-1.41 (m, 2H), 1.39-1.35 (m, 6H), 1.31-1.28 (m, 4H), 1.26 (s, 1H), 1.17-1.02 (m, 4H)

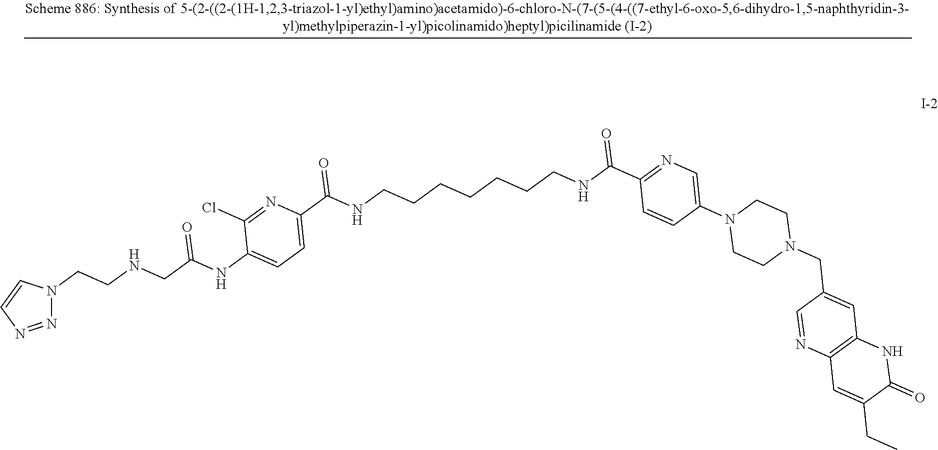 [SMILES: CCc1cc2ncc(CN3CCN(c4ccc(C(=O)NCCCCCCCNC(=O)c5ccc(NC(=O)CNCCn6ccnn6)c(Cl)n5)nc4)CC3)cc2[nH]c1=O]

LCMS (Method E): Rt=0.388 min, [M+H]⁺=812.5. ¹H NMR (400 MHz, DMSO-d6) δ=12.13-11.60 (m, 1H), 8.75 (d, J=8.4 Hz, 1H), 8.54-8.50 (m, 1H), 8.42-8.35 (m, 2H), 8.26 (d, J=2.8 Hz, 1H), 8.16 (d, J=0.8 Hz, 1H), 8.02 (d, J=8.4 Hz, 1H), 7.82 (d, J=8.8 Hz, 1H), 7.77-7.69 (m, 2H), 7.62 (d, J=1.2 Hz, 1H), 7.38-6.00 (m, 1H), 4.51-4.10 (m, 2H), 3.65 (s, 2H), 3.39 (s, 2H), 3.35-3.34 (m, 2H), 3.29-3.17 (m, 6H), 3.09-3.00 (m, 2H), 2.59-2.53 (m, 6H), 1.55-1.45 (m, 4H), 1.29 (s, 6H), 1.18-1.00 (m, 3H)

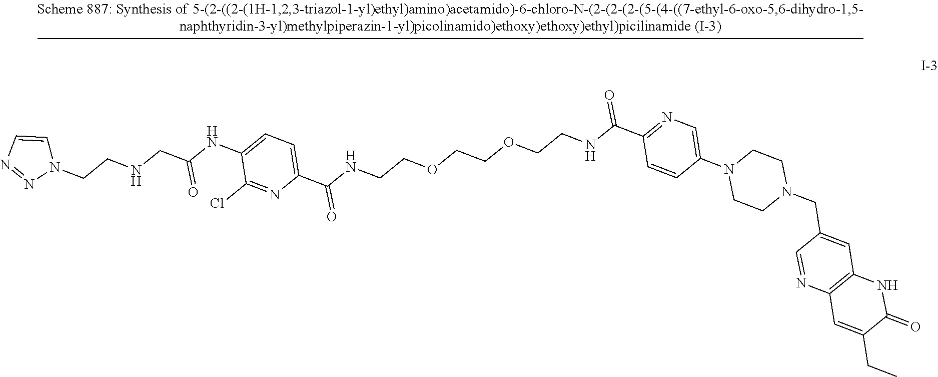 [SMILES: CCc1cc2ncc(CN3CCN(c4ccc(C(=O)NCCOCCOCCNC(=O)c5ccc(NC(=O)CNCCn6ccnn6)c(Cl)n5)nc4)CC3)cc2[nH]c1=O]

LCMS (Method E): Rt=0.374 min, [M+H]⁺=830.5. ¹H NMR (400 MHz, METHANOL-d4) δ=8.75 (d, J=8.0 Hz, 1H), 8.48 (s, 1H), 8.14 (d, J=2.0 Hz, 1H), 8.05 (s, 1H), 7.96 (d, J=8.4 Hz, 1H), 7.85-7.79 (m, 2H), 7.75 (d, J=9.2 Hz, 2H), 7.28-7.26 (m, 1H), 4.62-4.59 (m, 2H), 3.73 (s, 2H), 3.71-3.67 (m, 8H), 3.61-3.55 (m, 4H), 3.46 (s, 2H), 3.35 (d, J=4.4 Hz, 4H), 3.24-3.21 (m, 2H), 2.69-2.63 (m, 6H), 1.30-1.27 (m, 3H)1

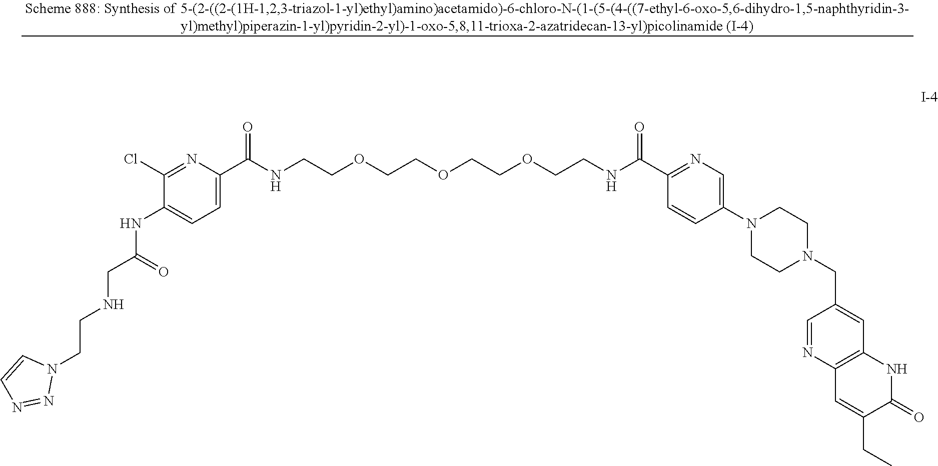 [SMILES: CCc1cc2ncc(CN3CCN(c4ccc(C(=O)NCCOCCOCCOCCNC(=O)c5ccc(NC(=O)CNCCn6ccnn6)c(Cl)n5)nc4)CC3)cc2[nH]c1=O]

LCMS (Method E): Rt=0.371 min, [M+H]⁺=874.5. ¹H NMR (400 MHz, METHANOL-d4) δ=9.43 (d, J=8.4 Hz, 1H), 9.13 (s, 1H), 8.86 (d, J=2.4 Hz, 1H), 8.71 (s, 1H), 8.65 (d, J=8.4 Hz, 1H), 8.54-8.47 (m, 2H), 8.41 (d, J=7.2 Hz, 2H), 7.98-7.95 (m, 1H), 5.28-5.26 (m, 2H), 4.38 (s, 2H), 4.32-4.28 (m, 12H), 4.24-4.19 (m, 4H), 4.12 (s, 2H), 4.05-4.00 (m, 4H), 3.97 (s, 4H), 3.90-3.87 (m, 2H), 3.35 (s, 2H), 1.96-1.92 (m, 3H)1

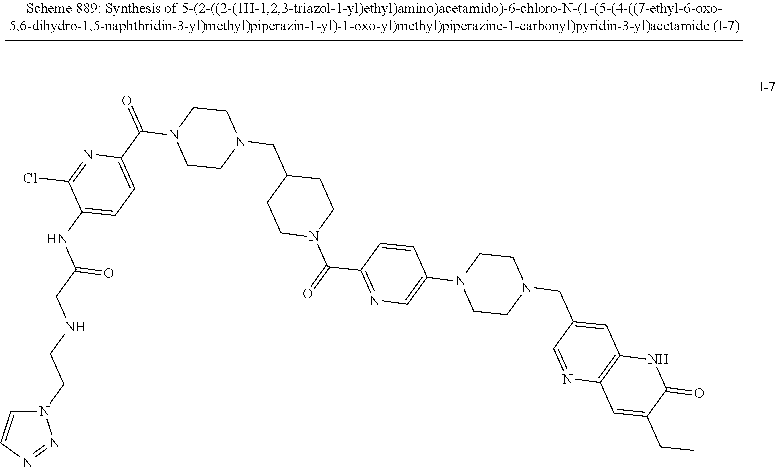 [SMILES: CCc1cc2ncc(CN3CCN(c4ccc(C(=O)N5CCC(CN6CCN(C(=O)c7ccc(NC(=O)CNCCn8ccnn8)c(Cl)n7)CC6)CC5)nc4)CC3)cc2[nH]c1=O]

LCMS: Rt: 0.374 min, [M+H]⁺=918.5. ¹H NMR (400 MHz, METHANOL-d₄) δ=8.77 (d, J=8.4 Hz, 1H), 8.49 (s, 1H), 8.24 (d, J=2.4 Hz, 1H), 8.15 (s, 1H), 8.08 (s, 1H), 8.01 (d, J=8.4 Hz, 1H), 7.88 (d, J=8.8 Hz, 1H), 7.83 (s, 1H), 7.80-7.74 (m, 2H), 7.34-7.32 (m, 1H), 4.66-4.44 (m, 2H), 3.77 (s, 2H), 3.68-3.61 (m, 16H), 3.61-3.54 (m, 6H), 3.40 (br s, 4H), 3.32-3.27 (m, 2H), 2.75-2.63 (m, 6H), 1.31-1.28 (m, 3H)

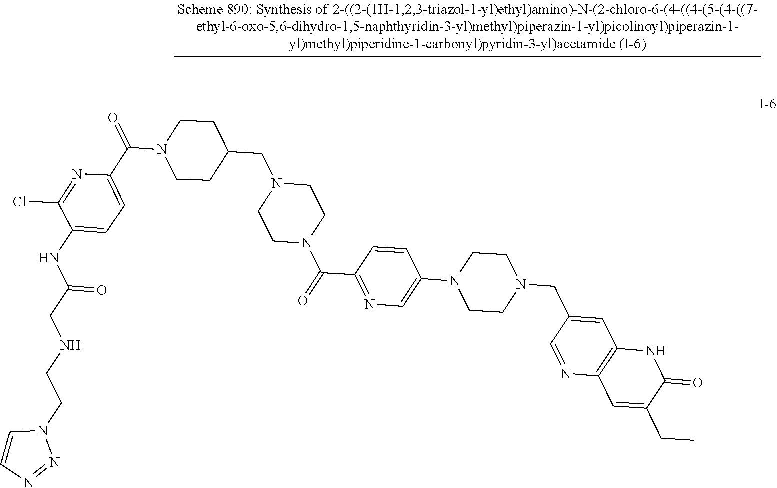 [SMILES: CCc1cc2ncc(CN3CCN(c4ccc(C(=O)N5CCN(CC6CCN(C(=O)c7ccc(NC(=O)CNCCn8ccnn8)c(Cl)n7)CC6)CC5)nc4)CC3)cc2[nH]c1=O]

LCMS (Method E): Rt=0.350 min, [M+H]⁺=865.1

¹H NMR (400 MHz, METHANOL-d₄) δ=8.77 (d, J=8.4 Hz, 1H), 8.50 (d, J=1.6 Hz, 1H), 8.24 (d, J=2.8 Hz, 1H), 8.06 (d, J=0.8 Hz, 1H), 7.84 (s, 1H), 7.78-7.74 (m, 2H), 7.57 (d, J=8.4 Hz, 1H), 7.53-7.47 (m, 1H), 7.46-7.39 (m, 1H), 4.63-4.59 (m, 3H), 3.92-3.81 (m, 1H), 3.74 (s, 4H), 3.61 (br s, 2H), 3.47 (s, 2H), 3.41-3.36 (m, 4H), 3.26-3.22 (m, 2H), 3.18-3.06 (m, 1H), 2.94-2.83 (m, 1H), 2.73-2.62 (m, 6H), 2.53 (br s, 2H), 2.49-2.38 (m, 2H), 2.28 (br d, J=8.0 Hz, 2H), 1.99-1.86 (m, 2H), 1.85-1.76 (m, 1H), 1.32-1.22 (m, 5H)

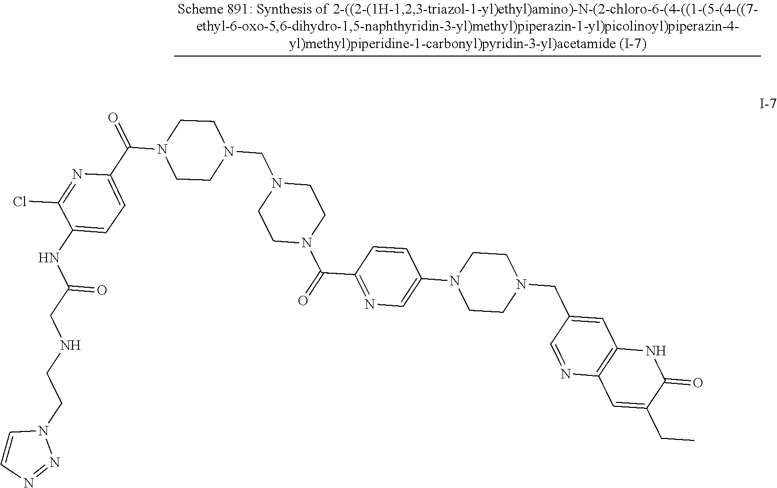 [SMILES: CCc1cc2ncc(CN3CCN(c4ccc(C(=O)N5CCN(CN6CCN(C(=O)c7ccc(NC(=O)CNCCn8ccnn8)c(Cl)n7)CC6)CC5)nc4)CC3)cc2[nH]c1=O]

LCMS (Method E): Rt=0.337 min, [M+H]⁺=865.5. ¹H NMR (400 MHz, METHANOL-d₄) δ=8.77 (d, J=8.4 Hz, 1H), 8.50 (d, J=2.0 Hz, 1H), 8.23 (d, J=2.4 Hz, 1H), 8.05 (d, J=0.8 Hz, 1H), 7.84 (s, 1H), 7.78-7.74 (m, 2H), 7.61 (d, J=8.4 Hz, 1H), 7.51-7.37 (m, 2H), 4.63-4.60 (m, 3H), 3.97-3.84 (m, 1H), 3.82-3.69 (m, 4H), 3.58 (br s, 2H), 3.47 (s, 2H), 3.41-3.35 (m, 4H), 3.25-3.22 (m, 2H), 3.17-3.06 (m, 1H), 2.93-2.81 (m, 1H), 2.76-2.61 (m, 6H), 2.59-2.39 (m, 4H), 2.28 (br d, J=6.8 Hz, 2H), 1.98-1.84 (m, 2H), 1.83-1.70 (m, 1H), 1.32-1.13 (m, 5H)

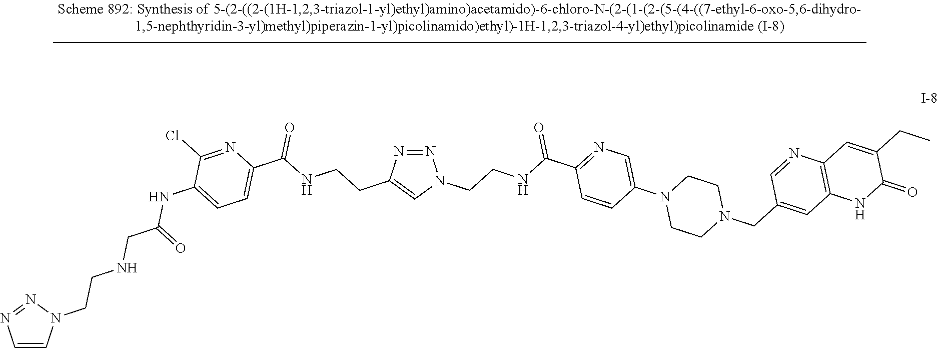 [SMILES: CCc1cc2ncc(CN3CCN(c4ccc(C(=O)NCCn5cc(CCNC(=O)c6ccc(NC(=O)CNCCn7ccnn7)c(Cl)n6)nn5)nc4)CC3)cc2[nH]c1=O]

LCMS (Method E): Rt=0.352 min, [M+H]⁺=837.6. ¹H NMR (400 MHz, DMSO-d6) δ=11.98-11.68 (m, 1H), 8.78-8.69 (m, 2H), 8.59-8.50 (m, 1H), 8.40 (d, J=1.6 Hz, 1H), 8.26 (d, J=2.4 Hz, 1H), 8.16 (d, J=0.4 Hz, 1H), 8.03 (d, J=8.4 Hz, 1H), 7.90 (s, 1H), 7.81 (d, J=8.8 Hz, 1H), 7.77-7.71 (m, 2H), 7.62 (s, 1H), 7.38-7.30 (m, 1H), 4.54-4.48 (m, 4H), 3.73-3.63 (m, 4H), 3.55-3.48 (m, 2H), 3.39 (s, 2H), 3.36-3.35 (m, 2H), 3.10-3.00 (m, 2H), 2.91-2.80 (m, 3H), 2.54 (d, J=6.4 Hz, 7H), 1.18-1.00 (m, 3H)

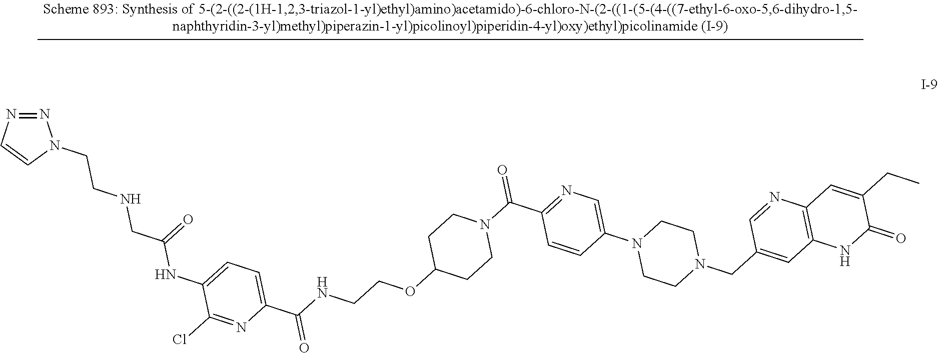 [SMILES: CCc1cc2ncc(CN3CCN(c4ccc(C(=O)N5CCC(OCCNC(=O)c6ccc(NC(=O)CNCCn7ccnn7)c(Cl)n6)CC5)nc4)CC3)cc2[nH]c1=O]

LCMS: Rt: 0.370 min, [M+H]⁺=826.4. ¹H NMR (400 MHz, METHANOL-d₄) δ=8.96 (d, J=1.2 Hz, 1H), 8.64 (d, J=8.4 Hz, 1H), 8.59-8.50 (m, 2H), 8.24 (s, 1H), 8.15-8.06 (m, 2H), 8.01-7.89 (m, 3H), 4.98-4.96 (m, 2H), 4.79 (s, 2H), 4.30 (s, 2H), 4.11-3.46 (m, 19H), 2.78-2.76 (m, 2H), 1.97 (br s, 2H), 1.76 (br d, J=4.4 Hz, 2H), 1.35-1.32 (m, 3H)

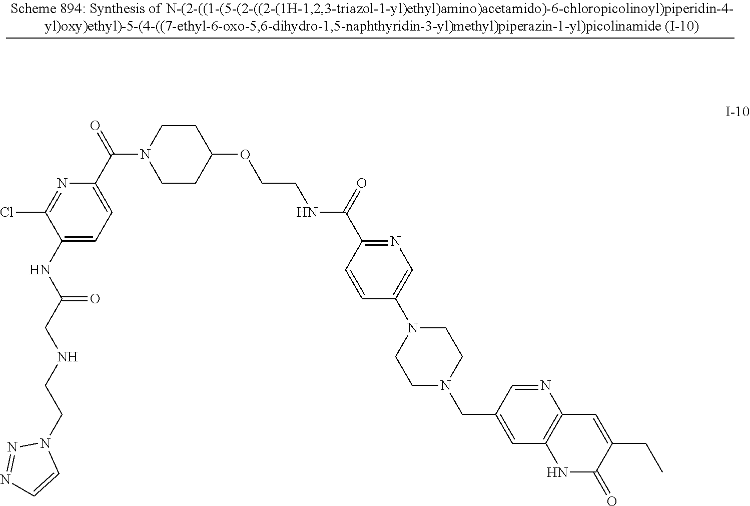 [SMILES: CCc1cc2ncc(CN3CCN(c4ccc(C(=O)NCCOC5CCN(C(=O)c6ccc(NC(=O)CNCCn7ccnn7)c(Cl)n6)CC5)nc4)CC3)cc2[nH]c1=O]

LCMS (Method E): Rt=0.374 min, [M+H]⁺=826.5. ¹H NMR (400 MHz, METHANOL-d4) δ=9.39 (d, J=8.4 Hz, 1H), 9.12 (s, 1H), 8.92 (d, J=2.4 Hz, 1H), 8.69 (s, 1H), 8.55 (d, J=8.8 Hz, 1H), 8.46 (s, 1H), 8.39 (d, J=11.6 Hz, 2H), 8.20 (d, J=8.4 Hz, 1H), 8.01-7.98 (m, 1H), 5.26-5.23 (m, 2H), 4.60-4.52 (m, 1H), 4.38-4.29 (m, 6H), 4.26-4.19 (m, 3H), 4.10 (s, 2H), 4.06-4.01 (m, 4H), 3.95 (s, 5H), 3.88-3.85 (m, 2H), 3.30-3.26 (m, 2H), 2.62-2.49 (m, 2H), 2.39-2.26 (m, 2H), 1.94-1.90 (m, 3H)1

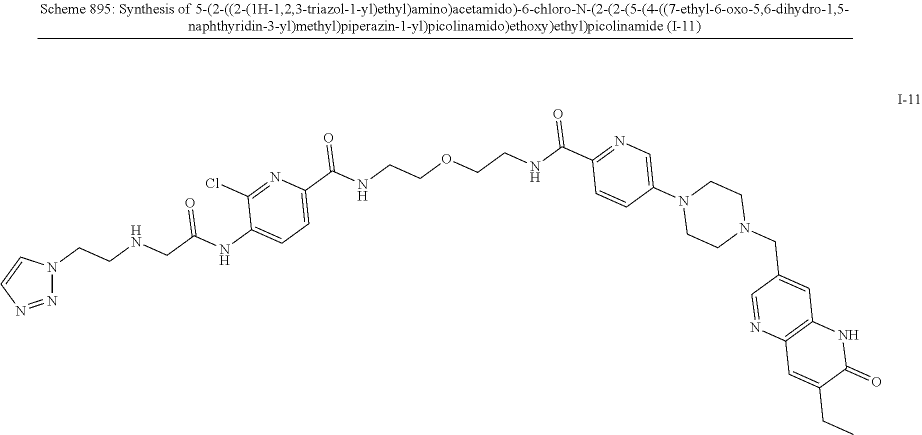 [SMILES: CCc1cc2ncc(CN3CCN(c4ccc(C(=O)NCCOCCNC(=O)c5ccc(NC(=O)CNCCn6ccnn6)c(Cl)n5)nc4)CC3)cc2[nH]c1=O]

LCMS: Rt=0.368 min, [M+H]⁺=786.6. ¹H NMR (400 MHz, DMSO-d₆) δ=8.70 (d, J=8.4 Hz, 1H), 8.49-8.44 (m, 1H), 8.40 (d, J=1.6 Hz, 1H), 8.34-8.30 (m, 1H), 8.27 (s, 1H), 8.10 (d, J=0.4 Hz, 2H), 7.97 (d, J=8.4 Hz, 1H), 7.79-7.74 (m, 2H), 7.70 (d, J=0.4 Hz, 1H), 7.64 (d, J=0.8 Hz, 1H), 7.35-7.31 (m, 1H), 4.50-4.46 (m, 2H), 3.63 (s, 2H), 3.58-3.53 (m, 4H), 3.46-3.40 (m, 4H), 3.35 (s, 2H), 3.28 (s, 4H), 3.08-3.04 (m, 2H), 2.56-2.51 (m, 6H), 1.18-1.13 (m, 3H)

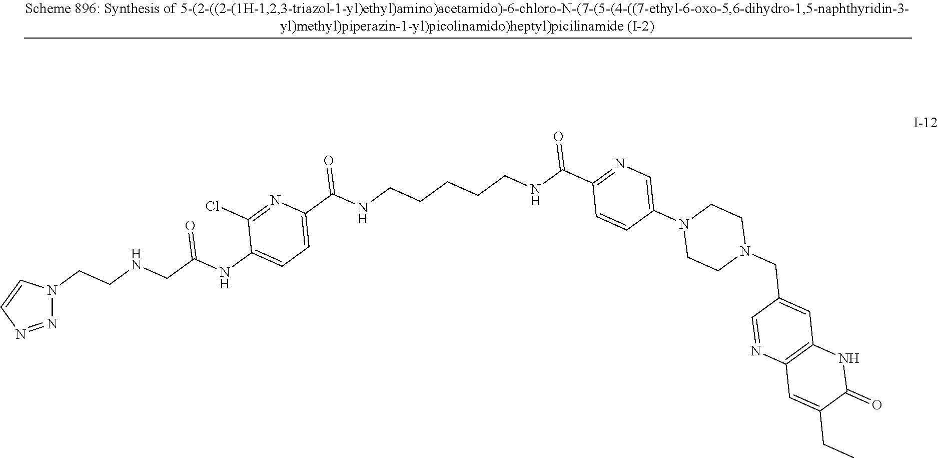 [SMILES: CCc1cc2ncc(CN3CCN(c4ccc(C(=O)NCCCCCNC(=O)c5ccc(NC(=O)CNCCn6ccnn6)c(Cl)n5)nc4)CC3)cc2[nH]c1=O]

LCMS (Method E): Rt=0.379 min, [M+H]⁺=784.4. ¹H NMR (400 MHz, METHANOL-d4) δ=9.43 (d, J=8.4 Hz, 1H), 9.12 (s, 1H), 8.85 (d, J=2.8 Hz, 1H), 8.69 (s, 1H), 8.63 (d, J=8.4 Hz, 1H), 8.53-8.45 (m, 2H), 8.39 (d, J=10.8 Hz, 2H), 7.98-7.95 (m, 1H), 5.26-5.23 (m, 2H), 4.37 (s, 2H), 4.10 (s, 2H), 4.06-4.00 (m, 8H), 3.95 (s, 4H), 3.89-3.86 (m, 2H), 3.30-3.27 (m, 2H), 2.34-2.27 (m, 4H), 2.13-2.06 (m, 2H), 1.94-1.90 (m, 3H)

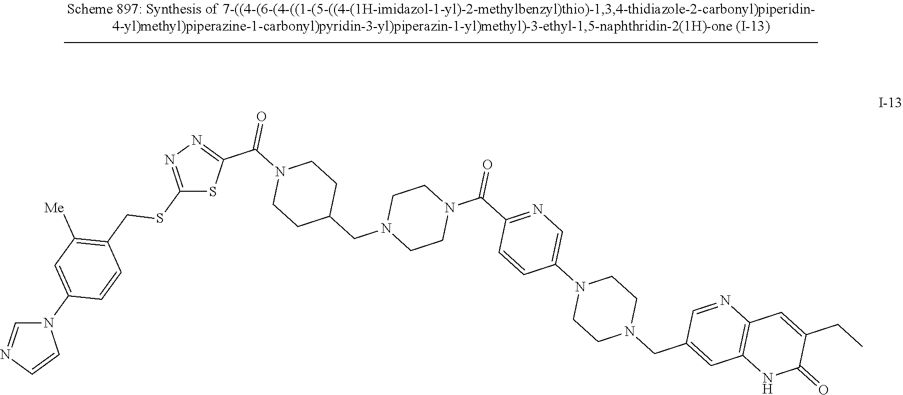 [SMILES: CCc1cc2ncc(CN3CCN(c4ccc(C(=O)N5CCN(CC6CCN(C(=O)c7nnc(SCc8ccc(-n9ccnc9)cc8C)s7)CC6)CC5)nc4)CC3)cc2[nH]c1=O]

LCMS (Method G): Rt: 0.557 min, [M+H]⁺=873.4. ¹H NMR (400 MHz, DMSO+D2O) δ=8.39 (d, J=1.6 Hz, 1H), 8.26-8.19 (m, 2H), 7.74 (s, 1H), 7.70 (s, 1H), 7.63 (d, J=1.2 Hz, 1H), 7.56-7.51 (m, 2H), 7.48-7.40 (m, 2H), 7.35-7.33 (m, 1H), 7.08 (s, 1H), 4.70-4.32 (m, 4H), 3.63 (s, 5H), 3.28 (br s, 4H), 3.23-3.13 (m, 1H), 2.86-2.83 (m, 1H), 2.57-2.50 (m, 7H), 2.44 (s, 3H), 2.41-2.26 (m, 4H), 2.14-2.13 (m, 2H), 1.92-1.71 (m, 3H), 1.19-1.00 (m, 5H)

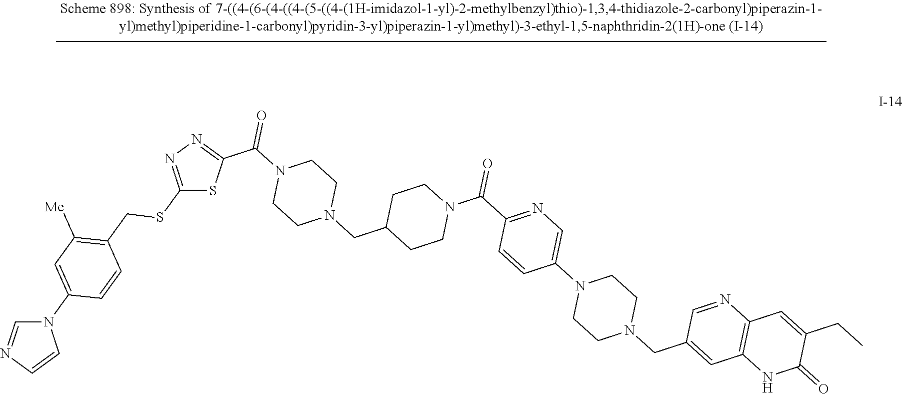 [SMILES: CCc1cc2ncc(CN3CCN(c4ccc(C(=O)N5CCC(CN6CCN(C(=O)c7nnc(SCc8ccc(-n9ccnc9)cc8C)s7)CC6)CC5)nc4)CC3)cc2[nH]c1=O]

LCMS (Method G): Rt: 0.586 min, [M+H]⁺=873.3. ¹H NMR (400 MHz, DMSO-d6) δ=11.86 (s, 1H), 8.40 (d, J=1.4 Hz, 1H), 8.25 (s, 2H), 7.74 (d, J=5.6 Hz, 2H), 7.66-7.53 (m, 3H), 7.50-7.33 (m, 3H), 7.09 (s, 1H), 4.68 (s, 2H), 4.53-4.39 (m, 1H), 4.03 (br s, 3H), 3.65 (s, 4H), 3.29 (br s, 4H), 3.07-2.95 (m, 1H), 2.83-2.67 (m, 1H), 2.58-2.52 (m, 6H), 2.46 (s, 3H), 2.43 (br s, 3H), 2.32-2.08 (m, 3H), 1.90-1.60 (m, 3H), 1.24-1.00 (m, 5H)

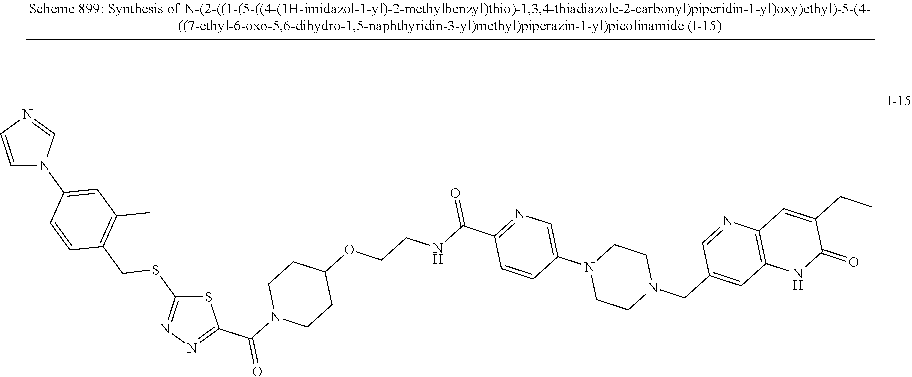 [SMILES: CCc1cc2ncc(CN3CCN(c4ccc(C(=O)NCCOC5CCN(C(=O)c6nnc(SCc7ccc(-n8ccnc8)cc7C)s6)CC5)nc4)CC3)cc2[nH]c1=O]

LCMS (Method G): Rt: 0.594 min, [M+H]⁺=834.2. ¹H NMR (400 MHz, DMSO-d6) δ=11.86 (s, 1H), 8.43-8.22 (m, 4H), 7.84 (d, J=8.8 Hz, 1H), 7.74 (d, J=4.4 Hz, 2H), 7.65-7.52 (m, 3H), 7.49-7.37 (m, 2H), 7.09 (s, 1H), 4.68 (s, 2H), 4.35-4.17 (m, 1H), 3.97-3.83 (m, 1H), 3.80-3.53 (m, 6H), 3.49-3.34 (m, 6H), 2.55 (t, J=6.8 Hz, 7H), 2.45 (s, 3H), 1.95-1.83 (m, 2H), 1.53-1.52 (m, 2H), 1.18 (t, J=7.4 Hz, 3H)

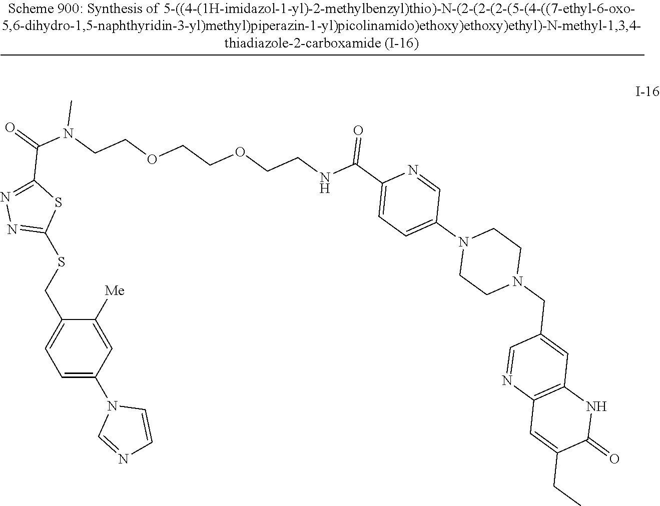 [SMILES: CCc1cc2ncc(CN3CCN(c4ccc(C(=O)NCCOCCOCCN(C)C(=O)c5nnc(SCc6ccc(-n7ccnc7)cc6C)s5)nc4)CC3)cc2[nH]c1=O]

LCMS (Method G): Rt: 0.560 min, [M+H]⁺=852.4. ¹H NMR (400 MHz, DMSO-d6) δ=11.85 (s, 1H), 8.39 (d, J=1.2 Hz, 1H), 8.31 (t, J=5.6 Hz, 1H), 8.27-8.21 (m, 2H), 7.82 (d, J=8.8 Hz, 1H), 7.73 (d, J=8.4 Hz, 2H), 7.62 (d, J=0.8 Hz, 1H), 7.54 (d, J=8.8 Hz, 2H), 7.47-7.42 (m, 1H), 7.37-7.35 (m, 1H), 7.09 (s, 1H), 4.66 (s, 2H), 4.01 (t, J=5.2 Hz, 1H), 3.70-3.57 (m, 5H), 3.57-3.41 (m, 7H), 3.41 (s, 2H), 3.40-3.33 (m, 3H), 3.05 (s, 2H), 2.59-2.51 (m, 7H), 2.44 (s, 3H), 1.18 (t, J=7.2 Hz, 3H)

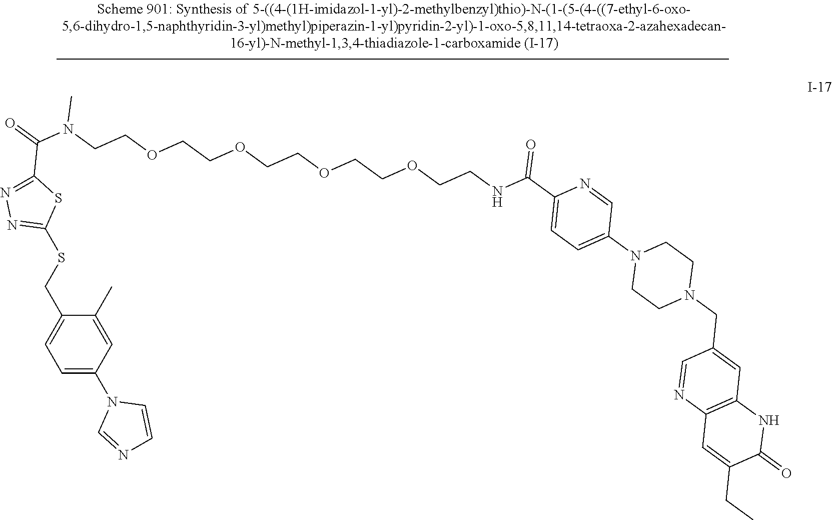 [SMILES: CCc1cc2ncc(CN3CCN(c4ccc(C(=O)NCCOCCOCCOCCOCCN(C)C(=O)c5nnc(SCc6ccc(-n7ccnc7)cc6C)s5)nc4)CC3)cc2[nH]c1=O]

LCMS (Method G): Rt: 0.569 mi, [M+H]⁺=940.5. ¹H NMR (400 MHz, DMSO-d6) δ=11.85 (s, 1H), 8.39 (d, J=1.6 Hz, 1H), 8.33 (t, J=5.8 Hz, 1H), 8.28-8.21 (m, 2H), 7.82 (d, J=8.8 Hz, 1H), 7.73 (d, J=6.4 Hz, 2H), 7.62 (d, J=0.8 Hz, 1H), 7.58-7.51 (m, 2H), 7.49-7.33 (m, 2H), 7.09 (s, 1H), 4.67 (s, 2H), 4.01 (t, J=5.4 Hz, 1H), 3.68-3.56 (m, 5H), 3.54-3.36 (m, 18H), 3.33-3.26 (m, 4H), 3.05 (s, 2H), 2.53 (d, J=6.4 Hz, 6H), 2.44 (s, 3H), 1.17 (t, J=7.4 Hz, 3H). ¹H NMR (400 MHz, DMSO+D2O) δ=8.38-8.34 (m, 1H), 8.17 (s, 2H), 7.78 (d, J=8.8 Hz, 1H), 7.71 (s, 1H), 7.63 (s, 2H), 7.49-7.43 (m, 2H), 7.38-7.27 (m, 2H), 7.06 (s, 1H), 4.55 (s, 2H), 3.91 (br s, 1H), 3.61-3.20 (m, 27H), 2.98 (s, 2H), 2.50-2.44 (m, 6H), 2.37 (s, 3H), 1.12 (t, J=7.4 Hz, 3H). ¹H NMR (400 MHz, DMSO+D2O, T=80° C.) δ=8.37 (s, 1H), 8.16 (d, J=2.4 Hz, 1H), 8.09 (s, 1H), 7.80 (d, J=8.8 Hz, 1H), 7.72 (s, 1H), 7.65 (s, 1H), 7.56 (s, 1H), 7.50-7.40 (m, 2H), 7.35-7.26 (m, 2H), 7.05 (s, 1H), 4.57 (s, 2H), 3.92 (d, J=1.6 Hz, 1H), 3.62 (s, 5H), 3.53-3.36 (m, 17H), 3.28 (d, J=4.4 Hz, 5H), 3.01 (br s, 2H), 2.53 (br s, 6H), 2.39 (s, 3H), 1.14 (t, J=7.4 Hz, 3H)

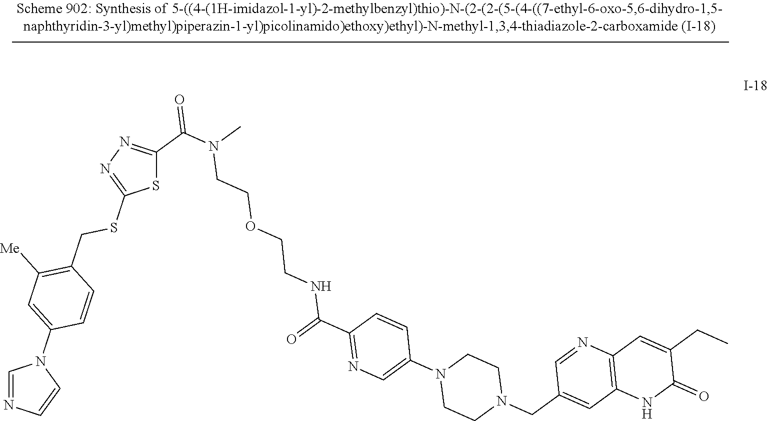 [SMILES: CCc1cc2ncc(CN3CCN(c4ccc(C(=O)NCCOCCN(C)C(=O)c5nnc(SCc6ccc(-n7ccnc7)cc6C)s5)nc4)CC3)cc2[nH]c1=O]

LCMS (Method G): Rt: 0.562 min, [M+H]⁺=808.4. ¹H NMR (400 MHz, DMSO-d6) δ=11.85 (br s, 1H), 8.39 (s, 1H), 8.34-8.28 (m, 1H), 8.27-8.19 (m, 2H), 7.81-7.78 (m, 1H), 7.76-7.70 (m, 2H), 7.62 (s, 1H), 7.57-7.51 (m, 2H), 7.48-7.41 (m, 1H), 7.41-7.34 (m, 1H), 7.09 (s, 1H), 4.66 (d, J=2.8 Hz, 2H), 4.06-3.97 (m, 1H), 3.65 (d, J=4.0 Hz, 5H), 3.56 (t, J=5.6 Hz, 1H), 3.51-3.44 (m, 2H), 3.42 (s, 2H), 3.40-3.33 (m, 4H), 3.05 (s, 2H), 2.58-2.52 (m, 7H), 2.44 (d, J=2.8 Hz, 3H), 1.21-1.13 (M, 3H)

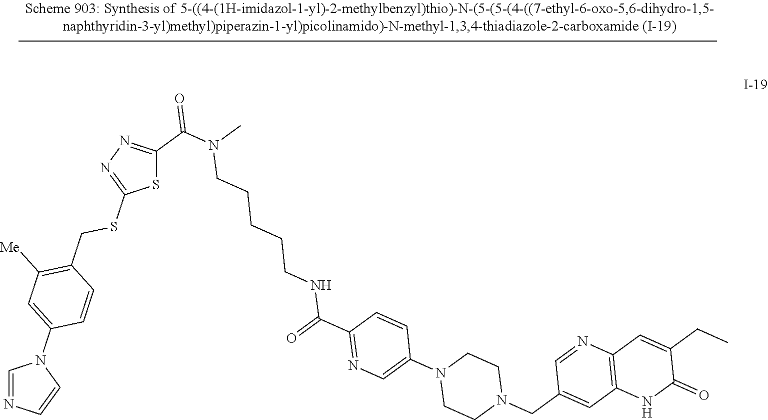 [SMILES: CCc1cc2ncc(CN3CCN(c4ccc(C(=O)NCCCCCN(C)C(=O)c5nnc(SCc6ccc(-n7ccnc7)cc6C)s5)nc4)CC3)cc2[nH]c1=O]

LCMS (Method G): Rt: 0.594 min, [M+H]⁺=806.3. ¹H NMR (400 MHz, DMSO-d6) δ=8.43-8.37 (m, 2H), 8.28-8.22 (m, 2H), 7.82-7.80 (m, 1H), 7.76-7.70 (m, 2H), 7.62 (s, 1H), 7.57-7.52 (m, 2H), 7.48-7.43 (m, 1H), 7.38-7.36 (m, 1H), 7.09 (s, 1H), 4.68 (s, 2H), 3.83-3.78 (m, 1H), 3.64-3.63 (m, 2H), 3.45 (t, J=7.2 Hz, 1H), 3.38-3.35 (m, 2H), 3.31-3.20 (m, 4H), 3.04-3.01 (m, 2H), 2.58-2.51 (m, 7H), 2.45 (d, J=6.4 Hz, 3H), 1.70-1.42 (m, 5H), 1.35-1.20 (m, 3H), 1.18 (t, J=7.2 Hz, 3H). ¹H NMR (400 MHz, DMSO-d6, T=80° C.) δ=8.39 (s, 1H), 8.21 (br s, 1H), 8.15 (s, 1H), 7.82 (d, J=8.8 Hz, 1H), 7.74 (s, 1H), 7.68-7.60 (m, 2H), 7.55-7.47 (m, 2H), 7.43-7.31 (m, 2H), 7.08 (s, 1H), 4.66-4.57 (m, 2H), 3.77 (br s, 1H), 3.64 (br s, 2H), 3.35-3.22 (m, 7H), 3.02 (br s, 2H), 2.55-2.54 (m, 7H), 2.46-2.42 (m, 3H), 1.73-1.41 (m, 5H), 1.41-1.12 (m, 6H)

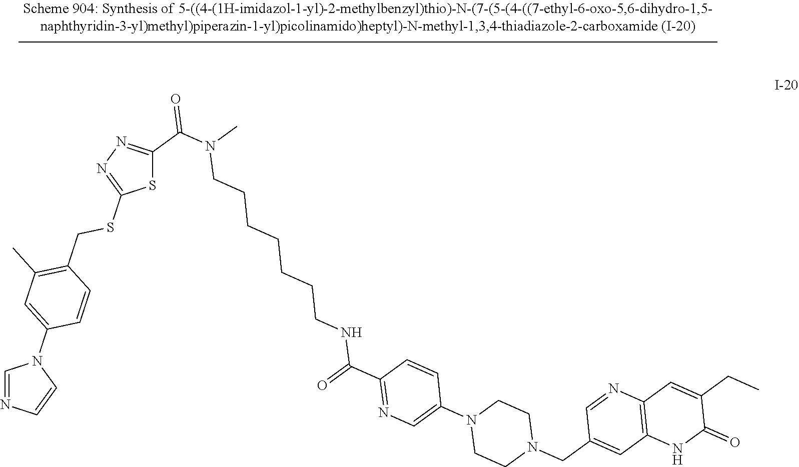 [SMILES: CCc1cc2ncc(CN3CCN(c4ccc(C(=O)NCCCCCCCN(C)C(=O)c5nnc(SCc6ccc(-n7ccnc7)cc6C)s5)nc4)CC3)cc2[nH]c1=O]

LCMS (Method E): Rt: 0.439 min, [M+H]⁺=834.3

¹H NMR (400 MHz, DMSO-d6) δ=11.85 (s, 1H), 8.43-8.33 (m, 2H), 8.30-8.18 (m, 2H), 7.82-7.80 (m, 1H), 7.77-7.70 (m, 2H), 7.62 (s, 1H), 7.58-7.51 (m, 2H), 7.49-7.33 (m, 2H), 7.09 (s, 1H), 4.68 (s, 2H), 3.79 (t, J=7.4 Hz, 1H), 3.64 (s, 2H), 3.44 (t, J=7.4 Hz, 1H), 3.35 (s, 2H), 3.32-3.29 (m, 4H), 3.24 (d, J=5.6 Hz, 2H), 3.01 (s, 1H), 2.54 (d, J=7.0 Hz, 6H), 2.45 (d, J=4.8 Hz, 3H), 1.67-1.43 (m, 4H), 1.36-1.14 (m, 9H). Special NMR: ¹H NMR (400 MHz, DMSO-d6, T=80° C.) δ=11.64 (br s, 1H), 8.41 (s, 1H), 8.29-8.13 (m, 3H), 7.84 (d, J=8.8 Hz, 1H), 7.74 (s, 1H), 7.66 (d, J=8.0 Hz, 2H), 7.59-7.50 (m, 2H), 7.47-7.34 (m, 2H), 7.10 (s, 1H), 4.69 (s, 2H), 3.81 (d, J=5.4 Hz, 1H), 3.67 (s, 2H), 3.48 (d, J=4.4 Hz, 1H), 3.35-3.32 (m, 1H), 3.29 (d, J=6.0 Hz, 2H), 3.14 (s, 4H), 3.04 (br s, 2H), 2.62-2.55 (m, 6H), 2.47 (s, 3H), 1.70-1.46 (m, 4H), 1.39-1.18 (m, 9H)

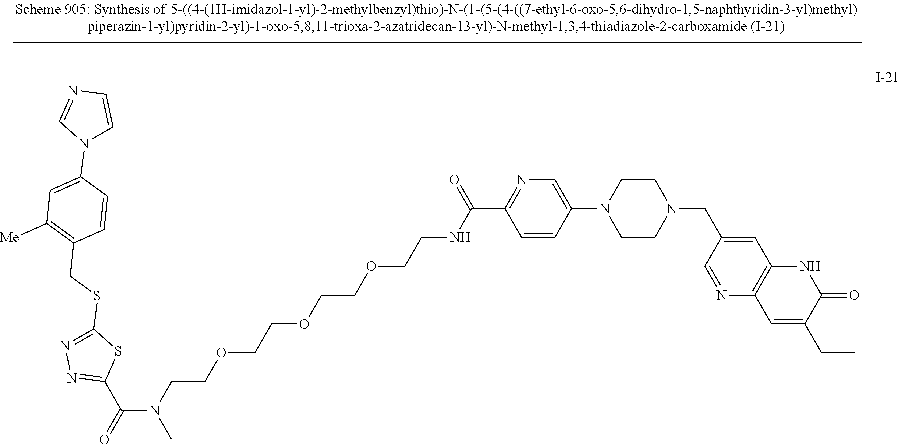 [SMILES: CCc1cc2ncc(CN3CCN(c4ccc(C(=O)NCCOCCOCCOCCN(C)C(=O)c5nnc(SCc6ccc(-n7ccnc7)cc6C)s5)nc4)CC3)cc2[nH]c1=O]

LCMS (Method G): Rt: 0.575 min, [M+H]⁺=896.5. ¹H NMR (400 MHz, DMSO-d6) δ=11.85 (s, 1H), 8.40 (s, 1H), 8.32-8.30 (m, 1H), 8.29-8.21 (m, 2H), 7.82 (d, J=8.8 Hz, 1H), 7.73 (d, J=8.8 Hz, 2H), 7.62 (s, 1H), 7.59-7.50 (m, 2H), 7.48-7.42 (m, 1H), 7.38 (d, J=7.2 Hz, 1H), 7.09 (s, 1H), 4.67 (s, 2H), 4.00 (t, J=5.2 Hz, 1H), 3.68-3.54 (m, 6H), 3.54-3.48 (m, 6H), 3.48-3.42 (m, 5H), 3.41 (br s, 2H), 3.36 (br s, 3H), 3.05 (s, 2H), 2.54 (d, J=6.8 Hz, 7H), 2.44 (s, 3H), 1.18 (t, J=7.2 Hz, 3H)

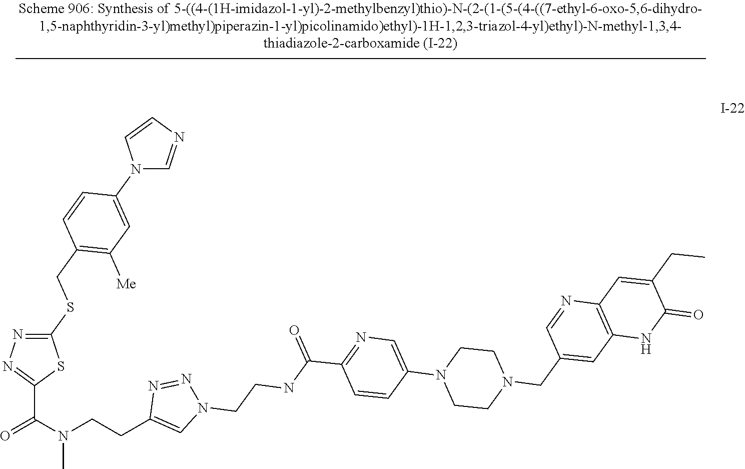

LCMS (Method E): Retention time: 0.399 min, [M+H]⁺=859.3. ¹H NMR (400 MHz, DMSO-d6) δ=11.84 (s, 1H), 8.57 (q, J=6.0 Hz, 1H), 8.40 (d, J=1.6 Hz, 1H), 8.28-8.21 (m, 2H), 7.93-7.78 (m, 2H), 7.73-7.72 (m, 2H), 7.62 (s, 1H), 7.57-7.50 (m, 2H), 7.47-7.41 (m, 1H), 7.37-7.35 (m, 1H), 7.09 (s, 1H), 4.67 (d, J=4.4 Hz, 2H), 4.49-4.47 (m, 2H), 4.09 (t, J=7.2 Hz, 1H), 3.73-3.64 (m, 3H), 3.64 (s, 2H), 3.36-3.33 (m, 2H), 3.31 (br s, 3H), 3.00 (s, 2H), 2.99-2.89 (m, 2H), 2.54 (d, J=7.2 Hz, 6H), 2.45 (d, J=4.0 Hz, 3H), 1.18 (t, J=7.2 Hz, 3H)

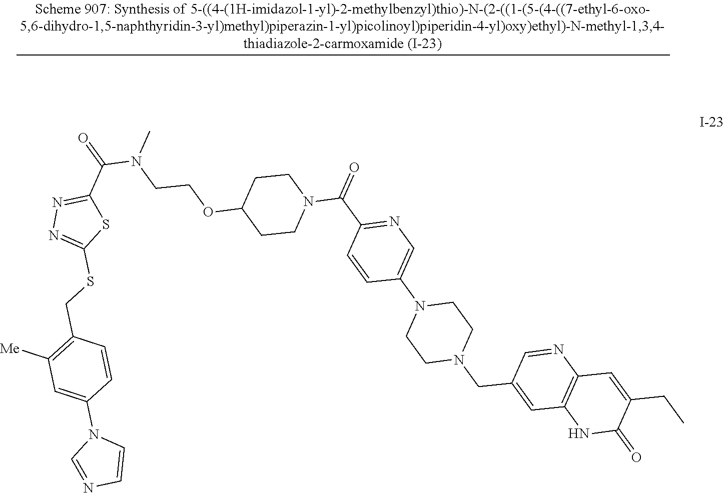 [SMILES: CCc1cc2ncc(CN3CCN(c4ccc(C(=O)N5CCC(OCCN(C)C(=O)c6nnc(SCc7ccc(-n8ccnc8)cc7C)s6)CC5)nc4)CC3)cc2[nH]c1=O]

LCMS (Method E): Retention time: 0.407 min, [M+H]⁺=848.5. ¹H NMR (400 MHz, DMSO-d6) δ=11.85 (s, 1H), 8.40 (s, 1H), 8.28-8.20 (m, 2H), 7.80-7.69 (m, 2H), 7.62 (s, 1H), 7.59-7.51 (m, 2H), 7.49-7.40 (m, 2H), 7.39-7.31 (m, 1H), 7.08 (s, 1H), 4.78-4.56 (m, 2H), 4.05 (br t, J=4.8 Hz, 1H), 3.97-3.71 (m, 2H), 3.71-3.56 (m, 6H), 3.56-3.48 (m, 1H), 3.43 (s, 1H), 3.32-3.25 (m, 7H), 3.06 (s, 2H), 2.58-2.52 (m, 5H), 2.47-2.38 (m, 3H), 1.92-1.60 (m, 2H), 1.53-1.26 (m, 2H), 1.18 (t, J=7.6 Hz, 3H)1

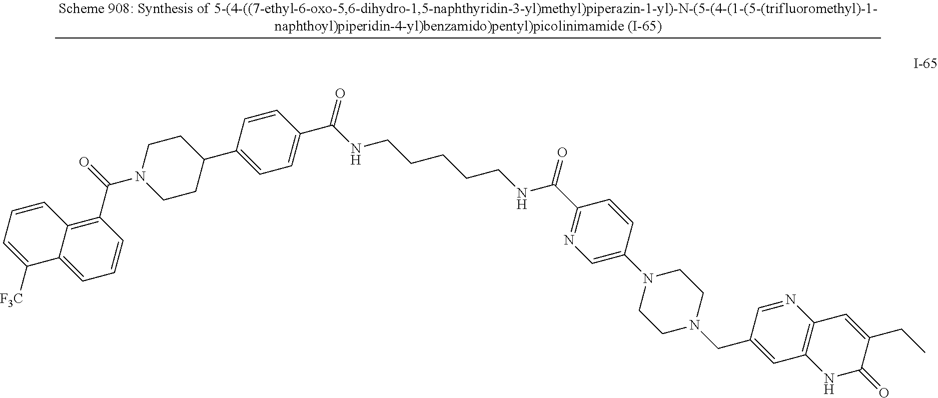 [SMILES: CCc1cc2ncc(CN3CCN(c4ccc(C(=O)NCCCCCNC(=O)c5ccc(C6CCN(C(=O)c7cccc8c(C(F)(F)F)cccc78)CC6)cc5)nc4)CC3)cc2[nH]c1=O]

LCMS: Rt=0.507 min, [M+H]⁺=887.5. ¹H NMR (400 MHz, DMSO-d6) δ=12.11-11.72 (m, 1H), 8.46-8.33 (m, 3H), 8.26 (br s, 1H), 8.24 (br s, 1H), 8.16-8.04 (m, 3H), 7.84-7.74 (m, 7H), 7.66-7.59 (m, 1H), 7.42-7.31 (m, 3H), 4.89-4.79 (m, 1H), 3.74-3.54 (m, 2H), 3.27-3.19 (m, 6H), 3.11-2.83 (m, 4H), 2.66-2.52 (m, 5H), 2.01-1.46 (m, 10H), 1.31 (m, 3H), 1.18 (m, 3H)

¹⁹F NMR (400 MHz, DMSO-d6)

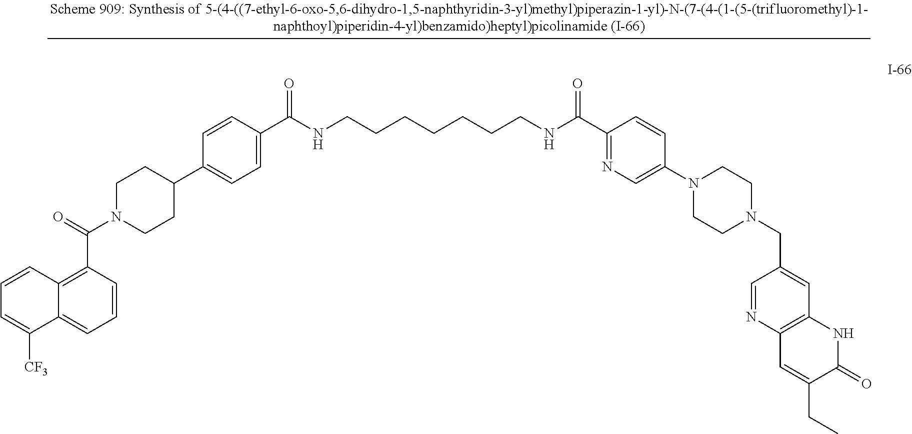 [SMILES: CCc1cc2ncc(CN3CCN(c4ccc(C(=O)NCCCCCCCNC(=O)c5ccc(C6CCN(C(=O)c7cccc8c(C(F)(F)F)cccc78)CC6)cc5)nc4)CC3)cc2[nH]c1=O]

LCMS: Rt=0.521 min, [M+H]⁺=915.4. ¹H NMR (400 MHz, DMSO-d6) δ=11.86 (br s, 1H), 8.45-8.33 (m, 3H), 8.25 (br s, 1H), 8.23 (s, 1H), 8.16-8.12 (m, 1H), 8.11-7.99 (m, 2H), 7.83-7.71 (m, 6H), 7.71-7.71 (m, 1H), 7.62 (br s, 1H), 7.34 (m, 3H), 4.84 (m, 1H), 3.64 (br s, 2H), 3.30-3.19 (m, 7H), 3.18-2.78 (m, 4H), 2.59-2.51 (m, 6H), 1.98-1.57 (m, 4H), 1.49 (br s, 4H), 1.29 (br s, 7H), 1.17 (m, 3H)

¹⁹F NMR (400 MHz, DMSO-d6)

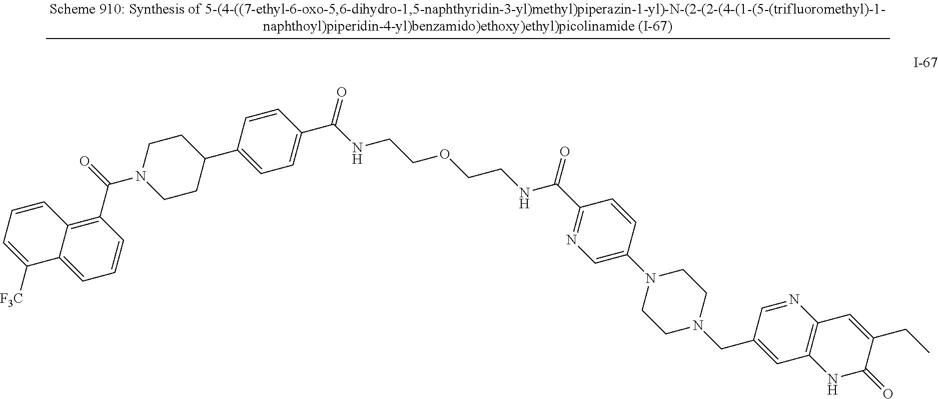 [SMILES: CCc1cc2ncc(CN3CCN(c4ccc(C(=O)NCCOCCNC(=O)c5ccc(C6CCN(C(=O)c7cccc8c(C(F)(F)F)cccc78)CC6)cc5)nc4)CC3)cc2[nH]c1=O]

LCMS: Rt=0.492 min, [M+H]⁺=889.5. ¹H NMR (400 MHz, DMSO-d6) δ=11.86 (br s, 1H), 8.45-8.36 (m, 3H), 8.25 (s, 1H), 8.23-8.21 (m, 1H), 8.13 (m, 1H), 8.10-7.97 (m, 2H), 7.86-7.70 (m, 7H), 7.61 (s, 1H), 7.41-7.31 (m, 3H), 4.94-4.77 (m, 1H), 3.63 (s, 2H), 3.58-3.52 (m, 4H), 3.47-3.38 (m, 6H), 2.91 (br s, 4H), 2.90-2.81 (m, 1H), 2.54 (m, 6H), 2.03-1.54 (m, 4H), 1.43-1.21 (m, 1H), 1.18 (m, 3H)

¹⁹F NMR (400 MHz, DMSO-d6)

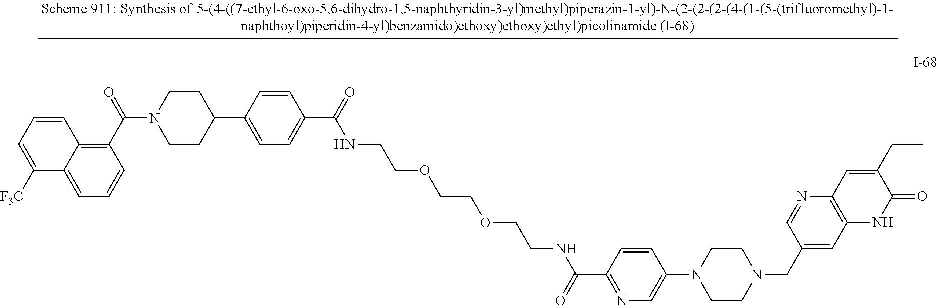 [SMILES: CCc1cc2ncc(CN3CCN(c4ccc(C(=O)NCCOCCOCCNC(=O)c5ccc(C6CCN(C(=O)c7cccc8c(C(F)(F)F)cccc78)CC6)cc5)nc4)CC3)cc2[nH]c1=O]

LCMS: Rt=0.495 min, [M+H]⁺=933.5. ¹H NMR (400 MHz, DMSO-d6) δ=12.00-11.76 (m, 1H), 8.45 (m, 1H), 8.40 (d, J=1.2 Hz, 1H), 8.36 (m, 1H), 8.28-8.26 (m, 1H), 8.24 (s, 1H), 8.15 (m, 1H), 8.12-7.99 (m, 2H), 7.85-7.74 (m, 7H), 7.63 (s, 1H), 7.40-7.32 (m, 3H), 4.85 (m, 1H), 3.64 (s, 2H), 3.57-3.51 (m, 8H), 3.45-3.40 (m, 5H), 3.31-2.95 (m, 5H), 2.90-2.83 (m, 1H), 2.58-2.53 (m, 6H), 1.97-1.57 (m, 4H), 1.38-1.21 (m, 1H), 1.18 (m, 3H)

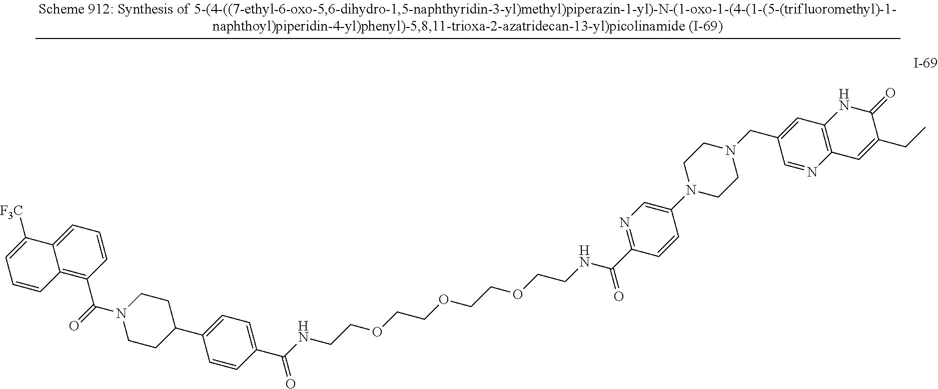 [SMILES: CCc1cc2ncc(CN3CCN(c4ccc(C(=O)NCCOCCOCCOCCNC(=O)c5ccc(C6CCN(C(=O)c7cccc8c(C(F)(F)F)cccc78)CC6)cc5)nc4)CC3)cc2[nH]c1=O]

LCMS: Rt=0.494 min, [M/2+H]⁺=489.4. ¹H NMR (400 MHz, DMSO-d6) δ=11.86 (br s, 1H), 8.44 (m, 1H), 8.40 (d, J=1.6 Hz, 1H), 8.33 (m, 1H), 8.27 (m, 1H), 8.24 (s, 1H), 8.14 (m, 1H), 8.10-8.02 (m, 2H), 7.84-7.73 (m, 7H), 7.62 (s, 1H), 7.39-7.32 (m, 3H), 4.84 (m, 1H), 3.63 (s, 2H), 3.55-3.47 (m, 14H), 3.43-3.40 (m, 3H), 3.28-2.91 (m, 5H), 2.90-2.83 (m, 1H), 2.58-2.52 (m, 6H), 2.00-1.58 (m, 4H), 1.39-. 1.21 (m, 1H), 1.18 (m, 3H).

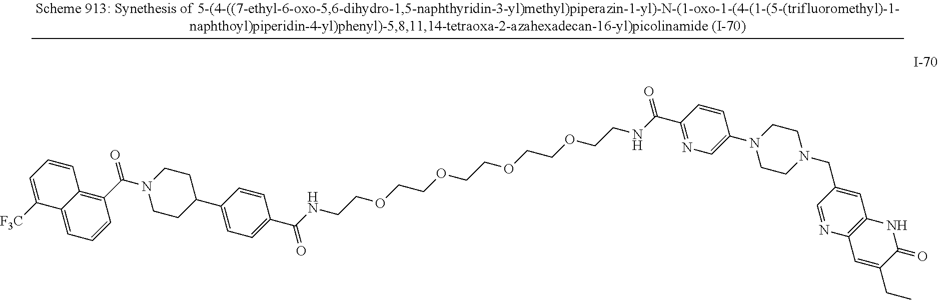 [SMILES: CCc1cc2ncc(CN3CCN(c4ccc(C(=O)NCCOCCOCCOCCOCCNC(=O)c5ccc(C6CCN(C(=O)c7cccc8c(C(F)(F)F)cccc78)CC6)cc5)nc4)CC3)cc2[nH]c1=O]

LCMS: Rt=0.495 min, [M+H]⁺=1021.6. ¹H NMR (400 MHz, DMSO-d6) δ=11.86 (br s, 1H), 8.44 (m, 1H), 8.39 (d, J=1.6 Hz, 1H), 8.34 (m, 1H), 8.26 (m, 1H), 8.23 (s, 1H), 8.14 (m, 1H), 8.11-8.01 (m, 2H), 7.85-7.74 (m, 7H), 7.62 (s, 1H), 7.39-7.32 (m, 3H), 4.84 (m, 1H), 3.63 (s, 2H), 3.49 (m, 18H), 3.44-3.39 (m, 4H), 3.28-2.96 (m, 4H), 2.91-2.83 (m, 1H), 2.56-2.51 (m, 6H), 1.97-1.56 (m, 4H), 1.49-1.20 (m, 1H), 1.17 (m, 3H).

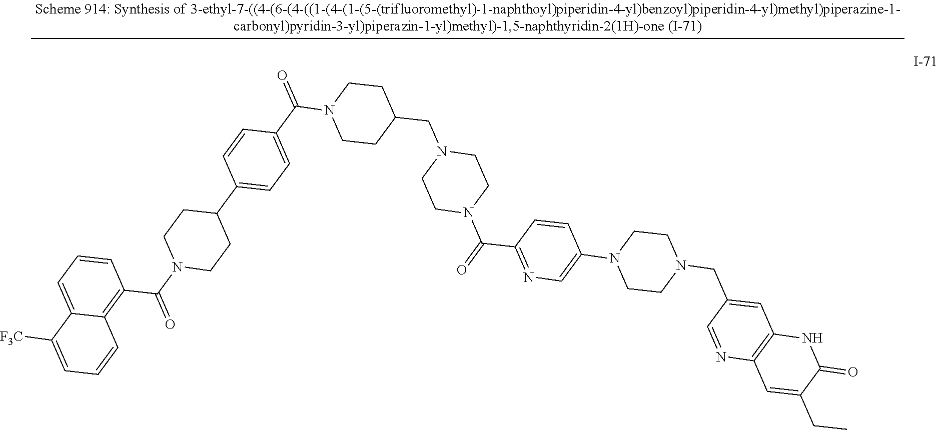 [SMILES: CCc1cc2ncc(CN3CCN(c4ccc(C(=O)N5CCN(CC6CCN(C(=O)c7ccc(C8CCN(C(=O)c9cccc%10c(C(F)(F)F)cccc9%10)CC8)cc7)CC6)CC5)nc4)CC3)cc2[nH]c1=O]

LCMS (Method G): Rt=0.680 min, [M+H]⁺=968.5. ¹H NMR (400 MHz, DMSO-d6) δ=11.86 (br s, 1H), 8.40 (d, J=1.6 Hz, 1H), 8.27-8.19 (m, 2H), 8.14 (br d, J=8.0 Hz, 1H), 8.09-8.04 (m, 1H), 7.83-7.71 (m, 4H), 7.63-7.60 (m, 1H), 7.50-7.45 (m, 1H), 7.39-7.27 (m, 5H), 4.88-4.80 (m, 1H), 4.54-4.32 (m, 1H), 3.64 (s, 2H), 3.59 (br s, 4H), 3.30 (br d, J=4.4 Hz, 3H), 3.24-3.13 (m, 2H), 3.11-2.81 (m, 5H), 2.80-2.69 (m, 1H), 2.54 (br d, J=6.4 Hz, 5H), 2.41-2.28 (m, 4H), 2.15 (br s, 2H), 2.00-1.91 (m, 1H), 1.87-1.57 (m, 6H), 1.38-1.20 (m, 1H), 1.19-1.15 (m, 3H), 1.10-0.95 (m, 2H).

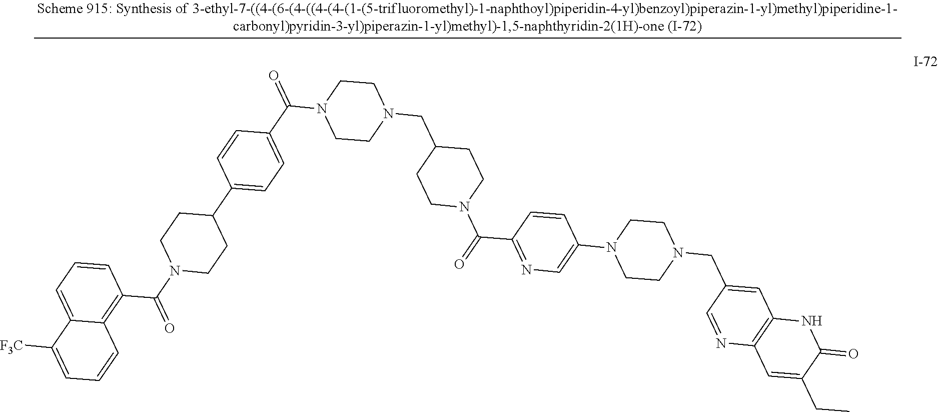 [SMILES: CCc1cc2ncc(CN3CCN(c4ccc(C(=O)N5CCC(CN6CCN(C(=O)c7ccc(C8CCN(C(=O)c9cccc%10c(C(F)(F)F)cccc9%10)CC8)cc7)CC6)CC5)nc4)CC3)cc2[nH]c1=O]

LCMS (Method G): Rt=0.675 min, [M+H]⁺=968.6. ¹H NMR (400 MHz, DMSO-d6) δ=11.86 (s, 1H), 8.40 (d, J=1.6 Hz, 1H), 8.25-8.22 (m, 1H), 8.17-8.12 (m, 1H), 8.09-8.04 (m, 1H), 7.84-7.60 (m, 5H), 7.44-7.40 (m, 1H), 7.39-7.28 (m, 5H), 4.93-4.76 (m, 1H), 4.45-4.42 (m, 1H), 4.09-3.93 (m, 1H), 3.64 (s, 2H), 3.61-3.50 (m, 2H), 3.29 (br d, J=4.4 Hz, 6H), 3.23-2.92 (m, 5H), 2.90-2.80 (m, 1H), 2.75-2.64 (m, 1H), 2.54 (br d, J=6.4 Hz, 6H), 2.42-2.24 (m, 4H), 2.16 (br s, 2H), 2.01-1.89 (m, 1H), 1.86-1.70 (m, 3H), 1.66-1.27 (m, 3H), 1.19-1.16 (m, 3H), 1.12-0.93 (m, 2H).

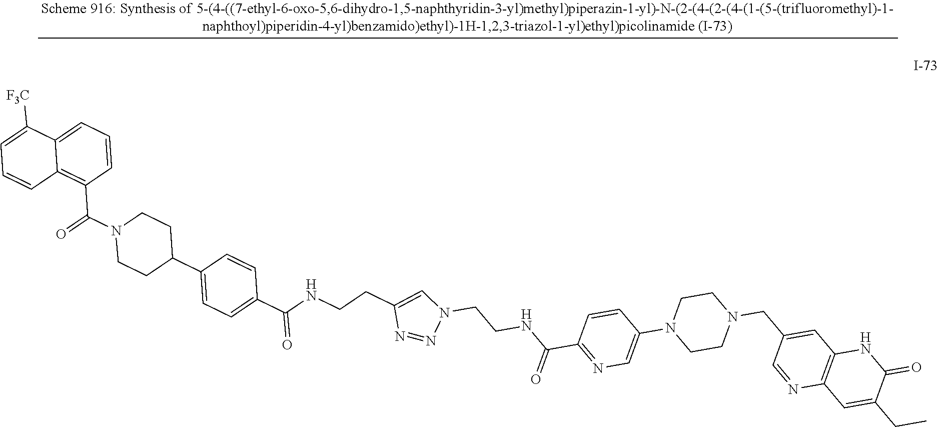 [SMILES: CCc1cc2ncc(CN3CCN(c4ccc(C(=O)NCCn5cc(CCNC(=O)c6ccc(C7CCN(C(=O)c8cccc9c(C(F)(F)F)cccc89)CC7)cc6)nn5)nc4)CC3)cc2[nH]c1=O]

LCMS (Method E): Rt=0.487 min, [M+H]⁺=940.6. ¹H NMR (400 MHz, DMSO-d6) δ=11.85 (s, 1H), 8.60-8.59 (m, 1H), 8.49-8.48 (m, 1H), 8.40 (d, J=2.0 Hz, 1H), 8.26 (s, 1H), 8.14 (br d, J=7.6 Hz, 1H), 8.09-7.73 (m, 9H), 7.63-7.60 (m, 1H), 7.40-7.31 (m, 3H), 4.88-4.81 (m, 1H), 4.51-4.49 (m, 2H), 3.70-3.69 (m, 2H), 3.64 (s, 2H), 3.52-3.41 (m, 2H), 3.30-3.24 (m, 2H), 3.23-2.92 (m, 3H), 2.91-2.80 (m, 3H), 2.54 (br d, J=6.4 Hz, 6H), 2.02-1.55 (m, 4H), 1.39-1.22 (m, 1H), 1.19-1.15 (m, 3H).

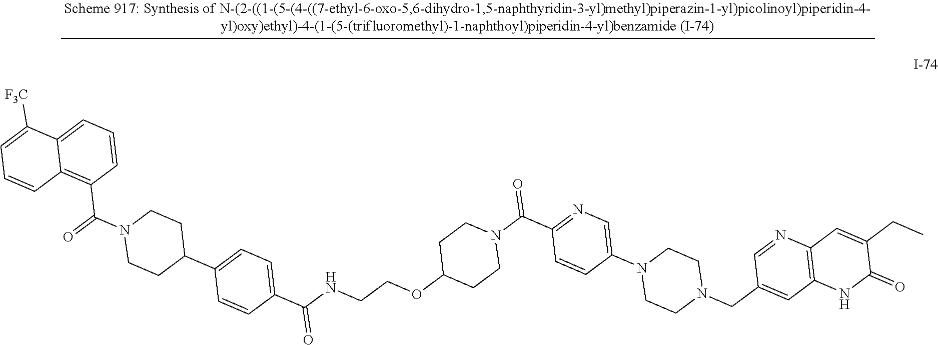 [SMILES: CCc1cc2ncc(CN3CCN(c4ccc(C(=O)N5CCC(OCCNC(=O)c6ccc(C7CCN(C(=O)c8cccc9c(C(F)(F)F)cccc89)CC7)cc6)CC5)nc4)CC3)cc2[nH]c1=O]

LCMS (Method G): Rt=0.626 min, [M+H]⁺=929.3. ¹H NMR (400 MHz, DMSO-d6) δ=12.05-11.67 (m, 1H), 8.46-8.45 (m, 1H), 8.40 (s, 1H), 8.28-8.22 (m, 1H), 8.18-8.11 (m, 1H), 8.10-7.72 (m, 7H), 7.63-7.60 (m, 1H), 7.47-7.42 (m, 1H), 7.36-7.34 (m, 3H), 4.93-4.76 (m, 1H), 4.01-3.88 (m, 1H), 3.85-3.72 (m, 1H), 3.64 (s, 2H), 3.55 (br s, 3H), 3.43-3.37 (m, 2H), 3.31-3.18 (m, 8H), 3.12-2.82 (m, 3H), 2.57-2.52 (m, 5H), 2.01-1.56 (m, 6H), 1.52-1.35 (m, 2H), 1.22-1.13 (m, 3H).

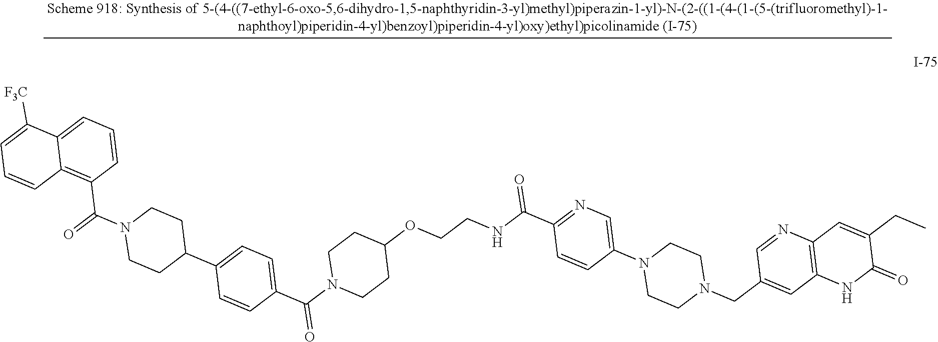 [SMILES: CCc1cc2ncc(CN3CCN(c4ccc(C(=O)NCCOC5CCN(C(=O)c6ccc(C7CCN(C(=O)c8cccc9c(C(F)(F)F)cccc89)CC7)cc6)CC5)nc4)CC3)cc2[nH]c1=O]

LCMS (Method G): Rt=0.678 min, [M+H]⁺=929.5. ¹H NMR (400 MHz, DMSO-d6) δ=11.99-11.65 (m, 1H), 8.42-8.33 (m, 2H), 8.29-8.22 (m, 1H), 8.14 (br d, J=8.4 Hz, 1H), 8.09-8.04 (m, 1H), 7.86-7.73 (m, 4H), 7.63-7.60 (m, 1H), 7.42-7.33 (m, 2H), 7.33-7.27 (m, 3H), 4.92-4.73 (m, 1H), 4.04-3.79 (m, 1H), 3.64 (s, 2H), 3.60-3.50 (m, 3H), 3.50-3.38 (m, 3H), 3.34 (br s, 3H), 3.27 (br d, J=13.2 Hz, 2H), 3.22-2.92 (m, 4H), 2.91-2.81 (m, 1H), 2.57-2.52 (m, 5H), 2.02-1.57 (m, 6H), 1.55-1.38 (m, 2H), 1.35 (br d, J=3.2 Hz, 1H), 1.19-1.16 (m, 3H).

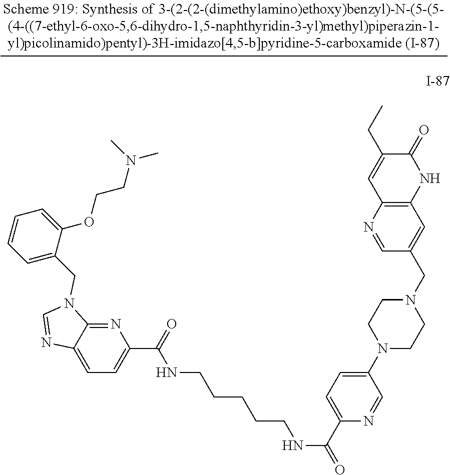 [SMILES: CCc1cc2ncc(CN3CCN(c4ccc(C(=O)NCCCCCNC(=O)c5ccc6ncn(Cc7ccccc7OCCN(C)C)c6n5)nc4)CC3)cc2[nH]c1=O]

LCMS (Method G): Rt=0.608 min, [M+H]⁺=800.4. ¹H NMR (400 MHz, DMSO-d6) δ=12.16 (br s, 1H), 9.07-8.99 (m, 2H), 8.72-8.68 (m, 2H), 8.52 (d, J=2.8 Hz, 1H), 8.46 (d, J=8.4 Hz, 1H), 8.24 (d, J=8.4 Hz, 1H), 8.11 (d, J=8.8 Hz, 1H), 8.05 (s, 1H), 7.92 (s, 1H), 7.79 (m, 1H), 7.66 (m, 1H), 7.61-7.53 (m, 1H), 7.32 (d, J=8.4 Hz, 1H), 7.19 (m, 1H), 5.81 (s, 2H), 4.36 (m, 2H), 3.95 (s, 2H), 3.69-3.63 (m, 4H), 3.61-3.55 (m, 4H), 2.89 (m, 2H), 2.87-2.82 (, 6H), 2.50 (s, 6H), 1.95-1.84 (m, 4H), 1.71-1.61 (m, 2H), 1.48 (m, 3H)

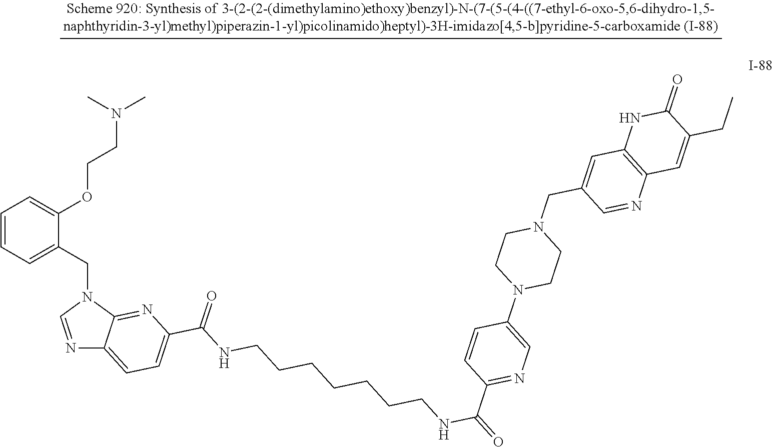 [SMILES: CCc1cc2ncc(CN3CCN(c4ccc(C(=O)NCCCCCCCNC(=O)c5ccc6ncn(Cc7ccccc7OCCN(C)C)c6n5)nc4)CC3)cc2[nH]c1=O]

LCMS (Method G): Rt=0.661 min, [M+H]⁺=828.5. ¹H NMR (400 MHz, DMSO-d6) δ=12.16 (br s, 1H), 9.09-9.00 (m, 2H), 8.74-8.67 (m, 2H), 8.55 (d, J=2.8 Hz, 1H), 8.46 (d, J=8.4 Hz, 1H), 8.25 (d, J=8.4 Hz, 1H), 8.12 (d, J=8.8 Hz, 1H), 8.06 (s, 1H), 7.93 (s, 1H), 7.81 (d, J=6.4 Hz, 1H), 7.68 (m, 1H), 7.60-7.52 (m, 1H), 7.31 (d, J=8.4 Hz, 1H), 7.18 (m, 1H), 5.83 (s, 2H), 4.35 (m, 2H), 3.95 (s, 2H), 3.70-3.65 (m, 3H), 3.56 (m, 5H), 2.90-2.83 (m, 8H), 2.81 (m, 6H), 1.91-1.78 (m, 4H), 1.68-1.58 (m, 6H), 1.49 (m, 3H)

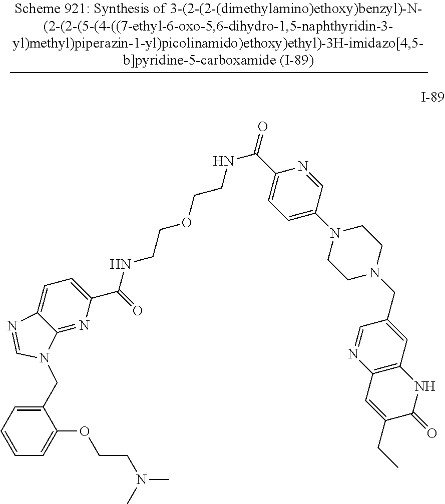 [SMILES: CCc1cc2ncc(CN3CCN(c4ccc(C(=O)NCCOCCNC(=O)c5ccc6ncn(Cc7ccccc7OCCN(C)C)c6n5)nc4)CC3)cc2[nH]c1=O]

LCMS (Method G): Rt=0.576 min, [M+H]⁺=802.5. ¹H NMR (400 MHz, DMSO-d6) δ=12.37-11.88 (m, 1H), 9.06-8.95 (m, 2H), 8.70-8.61 (m, 2H), 8.44 (d, J=8.4 Hz, 1H), 8.35 (d, J=2.8 Hz, 1H), 8.23 (d, J=8.4 Hz, 1H), 8.08-8.01 (m, 2H), 7.90 (s, 1H), 7.71 (m 1H), 7.60 (m, 1H), 7.56-7.49 (m, 1H), 7.28 (d, J=8.4 Hz, 1H), 7.15 (m, 1H), 5.74 (s, 2H), 4.33 (m, 2H), 3.93-3.88 (m, 5H), 3.82 (m, 2H), 3.79-3.72 (m, 2H), 3.60 (s, 3H), 3.55 (br s, 3H), 2.88-2.85 (m, 2H), 2.82 (m, 5H), 2.47 (s, 6H), 1.46 (m, 3H)

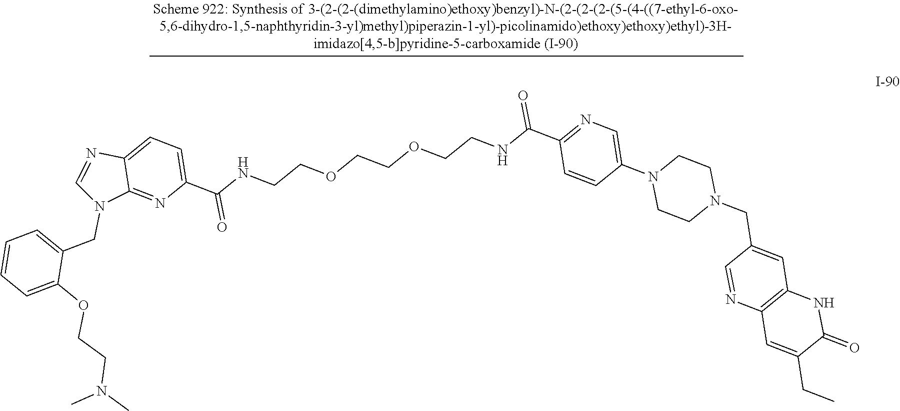 [SMILES: CCc1cc2ncc(CN3CCN(c4ccc(C(=O)NCCOCCOCCNC(=O)c5ccc6ncn(Cc7ccccc7OCCN(C)C)c6n5)nc4)CC3)cc2[nH]c1=O]

LCMS (Method G): Rt=0.580 min, [M+H]⁺=846.5. ¹H NMR (400 MHz, DMSO-d6) δ=11.85 (br s, 1H), 8.76-8.70 (m, 2H), 8.40 (d, J=1.6 Hz, 1H), 8.33 (m, 1H), 8.22 (d, J=2.8 Hz, 1H), 8.16 (d, J=8.4 Hz, 1H), 7.95 (d, J=8.4 Hz, 1H), 7.80 (d, J=8.8 Hz, 1H), 7.75 (s, 1H), 7.62 (s, 1H), 7.44 (m, 1H), 7.35 (m, 1H), 7.29-7.23 (m, 1H), 7.01 (d, J=8.4 Hz, 1H), 6.88 (m, 1H), 5.49 (s, 2H), 4.05 (m, 2H), 3.64-3.57 (m, 8H), 3.55-3.51 (m, 4H), 3.44-3.40 (m, 2H), 3.31-3.28 (m, 4H), 2.60-2.57 (m, 2H), 2.56-2.51 (m, 6H), 2.20 (s, 6H), 1.18 (m, 3H)

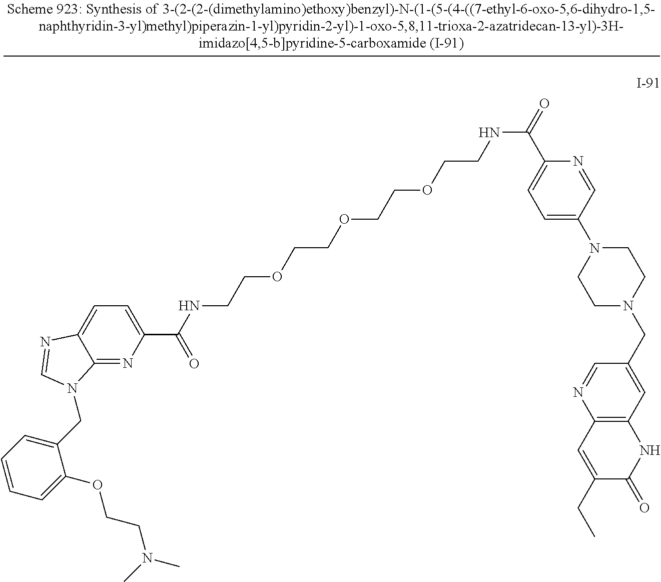

LCMS (Method G): Rt=0.586 min, [M+H]⁺=890.5. ¹H NMR (400 MHz, DMSO-d6) δ=11.86 (s, 1H), 8.77-8.68 (m, 2H), 8.40 (s, 1H), 8.34-8.30 (m, 1H), 8.26 (d, J=2.4 Hz, 1H), 8.18 (d, J=8.4 Hz, 1H), 7.97 (d, J=8.4 Hz, 1H), 7.83 (d, J=8.8 Hz, 1H), 7.76 (s, 1H), 7.62 (s, 1H), 7.45 (d, J=6.8 Hz, 1H), 7.40-7.36 (m, 1H), 7.30-7.26 (m, 1H), 7.03 (d, J=8.4 Hz, 1H), 6.92-6.88 (m, 1H), 5.52 (s, 2H), 4.10-4.08 (m, 2H), 3.69-3.45 (m, 17H), 3.44-3.34 (m, 5H), 2.66 (br s, 2H), 2.58-2.52 (m, 6H), 2.25 (s, 6H), 1.21-1.17 (m, 3H)

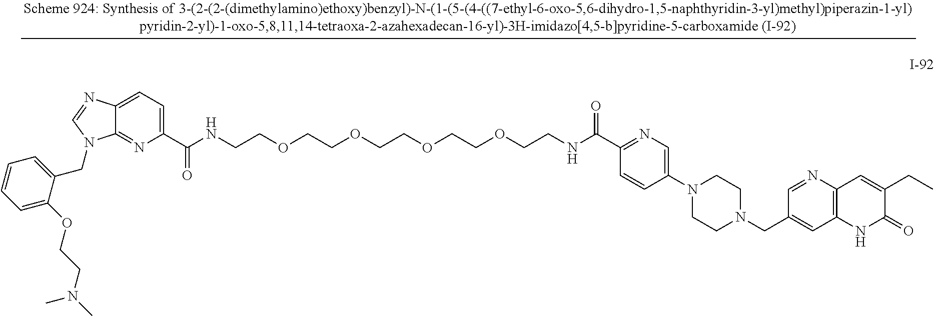

LCMS (Method G): Rt=0.559 min, [M+H]⁺=934.6. ¹H NMR (400 MHz, DMSO-d6) δ=11.89-11.80 (m, 1H), 8.76-8.69 (m, 2H), 8.39 (d, J=1.6 Hz, 1H), 8.35-8.29 (m, 1H), 8.26 (d, J=2.8 Hz, 1H), 8.17 (d, J=8.4 Hz, 1H), 7.96 (d, J=8.4 Hz, 1H), 7.86 (s, 1H), 7.75 (s, 1H), 7.62 (s, 1H), 7.48-7.42 (m, 1H), 7.38-7.36 (m, 1H), 7.31-7.24 (m, 1H), 7.02 (d, J=8.0 Hz, 1H), 6.92-6.86 (m, 1H), 5.50 (s, 2H), 4.07-4.05 (m, 2H), 3.67-3.38 (m, 24H), 2.61-2.51 (m, 10H), 2.20 (s, 6H), 1.20-1.16 (m, 3H)

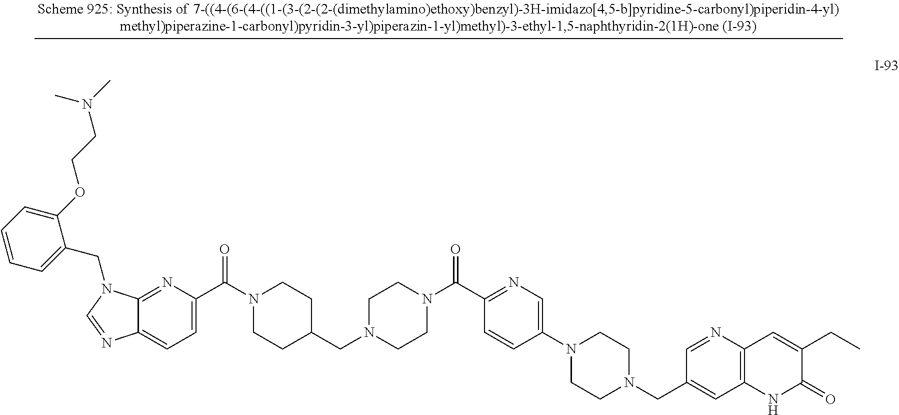

LCMS (Method G): Rt=0.602 min, [M+H]⁺=881.5. ¹H NMR (400 MHz, DMSO-d6) δ=11.84 (m, 1H), 8.68 (s, 1H), 8.40 (s, 1H), 8.25 (d, J=2.4 Hz, 1H), 8.15 (d, J=8.4 Hz, 1H), 7.75 (s, 1H), 7.62 (s, 1H), 7.48 (d, J=8.8 Hz, 1H), 7.43 (d, J=8.4 Hz, 1H), 7.37 (m, 1H), 7.27 (m, 1H), 7.11 (d, J=7.2 Hz, 1H), 7.04 (d, J=8.4 Hz, 1H), 6.86 (m, 1H), 5.42 (s, 2H), 4.49 (m, 1H), 4.09 (m, 2H), 3.71-3.64 (m, 3H), 3.61 (br s, 3H), 3.32-3.27 (m, 5H), 2.95 (m, 1H), 2.79 (m, 1H), 2.64 (m, 2H), 2.54 (m, 6H), 2.43-2.31 (m, 4H), 2.23 (s, 6H), 2.20-2.13 (m, 2H), 1.89-1.75 (m, 2H), 1.54 (m, 1H), 1.18 (m, 3H), 1.14-1.02 (m, 2H)

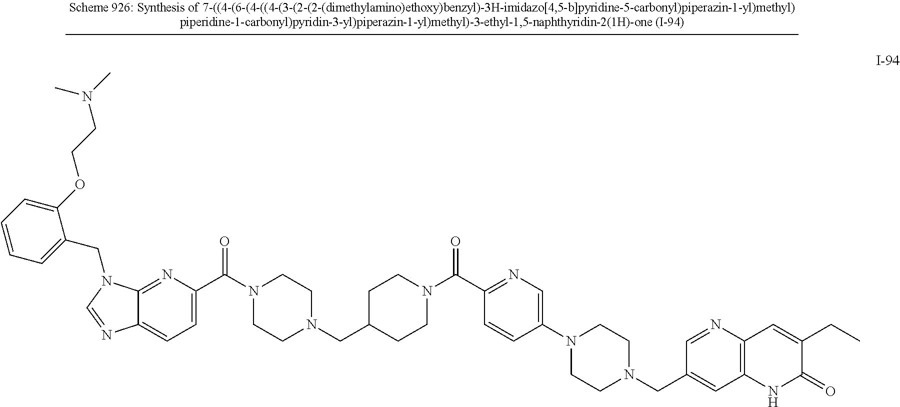

LCMS (Method G): Rt=0.596 min, [M+H]⁺=881.5. ¹H NMR (400 MHz, DMSO-d6) δ=11.84 (br s, 1H), 8.70 (s, 1H), 8.40 (d, J=1.6 Hz, 1H), 8.24 (d, J=2.4 Hz, 1H), 8.15 (d, J=8.4 Hz, 1H), 7.74 (s, 1H), 7.62 (s, 1H), 7.50-7.42 (m, 2H), 7.39-7.33 (m, 1H), 7.30-7.24 (m, 1H), 7.11 (d, J=7.6 Hz, 1H), 7.04 (d, J=8.4 Hz, 1H), 6.86 (m, 1H), 5.43 (s, 2H), 4.46 (m, 1H), 4.12-3.98 (m, 3H), 3.39 (br s, 2H), 3.37-3.22 (m, 8H), 3.09-2.95 (m, 1H), 2.81-2.70 (m, 1H), 2.63 (m, 2H), 2.54 (m, 6H), 2.47-2.37 (m, 2H), 2.27-2.21 (m, 8H), 2.17 (br s, 2H), 1.80 (m, 2H), 1.70-1.61 (m, 1H), 1.18 (m, 3H), 1.13-1.01 (m, 2H)

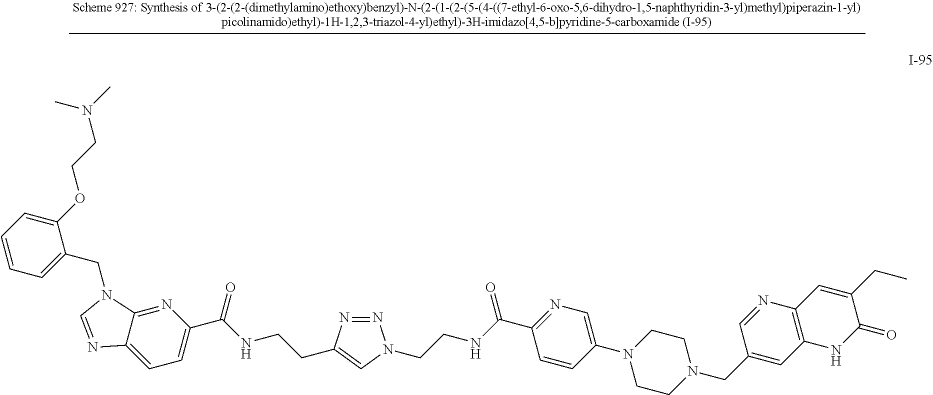

LCMS (Method G): Rt=0.533 min, [M+H]⁺=853.5. ¹H NMR (400 MHz, DMSO-d6) δ=11.85 (br s, 1H), 8.98 (m, 1H), 8.76 (s, 1H), 8.60 (m 1H), 8.39 (d, J=1.6 Hz, 1H), 8.23 (d, J=2.8 Hz, 1H), 8.17 (d, J=8.4 Hz, 1H), 7.98-7.92 (m, 2H), 7.80 (d, J=8.8 Hz, 1H), 7.75 (s, 1H), 7.62 (s, 1H), 7.51 (m, 1H), 7.35 (dm, 1H), 7.28-7.22 (m, 1H), 7.01 (d, J=8.4 Hz, 1H), 6.88 (m, 1H), 5.49 (s, 2H), 4.52 (m, 2H), 4.06 (m, 2H), 3.71 (m, 2H), 3.65-3.58 (m, 4H), 3.33-3.29 (m, 4H), 2.94 (m, 2H), 2.59 (m, 2H), 2.56 (br s, 6H), 2.20 (s, 6H), 1.18 (m, 3H)

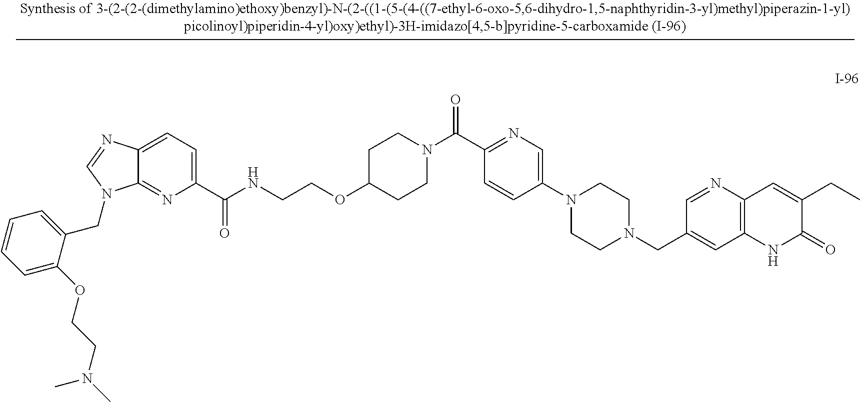

LCMS (Method G): Rt=0.539 min, [M+H]⁺=842.6. ¹H NMR (400 MHz, DMSO-d6) δ=11.85 (s, 1H), 8.79-8.63 (m, 2H), 8.40 (d, J=1.6 Hz, 1H), 8.19 (d, J=8.4 Hz, 2H), 7.97 (d, J=8.4 Hz, 1H), 7.75 (s, 1H), 7.62 (s, 1H), 7.45-7.33 (m, 3H), 7.25 (m, 1H), 7.01 (d, J=8.4 Hz, 1H), 6.87 (m, 1H), 5.50 (s, 2H), 4.06 (m, 2H), 4.01-3.90 (m, 1H), 3.88-3.76 (m, 1H), 3.68-3.61 (m, 4H), 3.53 (m, 2H), 3.31-3.19 (m, 7H), 2.60 (m, 2H), 2.54 (m, 6H), 2.21 (s, 6H), 1.97-1.76 (m, 2H), 1.46 (m, 2H), 1.18 (m, 3H)

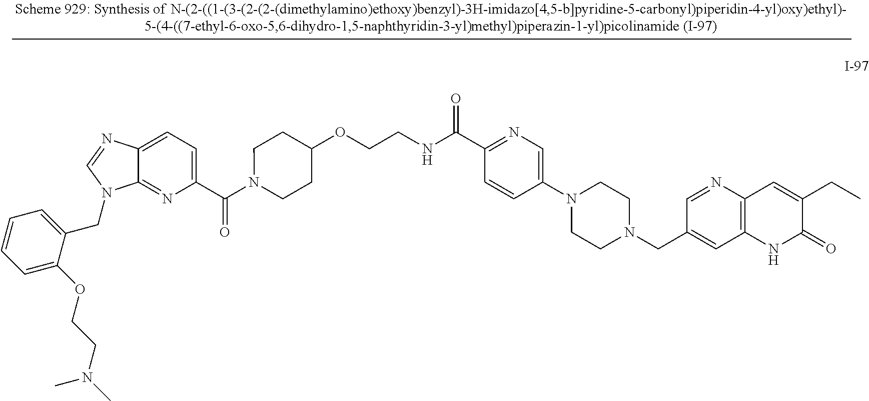

LCMS (Method G): Rt=0.554 min, [M+H]⁺=842.3. ¹H NMR (400 MHz, DMSO-d6) δ=11.84 (br s, 1H), 8.68 (s, 1H), 8.42-8.35 (m, 2H), 8.26 (d, J=2.8 Hz, 1H), 8.14 (d, J=8.4 Hz, 1H), 7.83 (d, J=8.8 Hz, 1H), 7.75 (s, 1H), 7.62 (s, 1H), 7.44 (d, J=8.4 Hz, 1H), 7.38 (m, 1H), 7.29-7.19 (m, 1H), 7.10 (d, J=7.6 Hz, 1H), 7.01 (d, J=8.4 Hz, 1H), 6.83 (m, 1H), 5.41 (s, 2H), 4.06 (m, 2H), 4.03-3.94 (m, 1H), 3.67-3.53 (m, 6H), 3.48-3.42 (m, 2H), 3.32-3.26 (m, 5H), 3.13 (m, 1H), 2.61 (m, 2H), 2.57-2.51 (m, 6H), 2.20 (s, 6H), 1.96-1.86 (m, 1H), 1.77-1.66 (m, 1H), 1.55-1.36 (m, 2H), 1.18 (m, 3H)

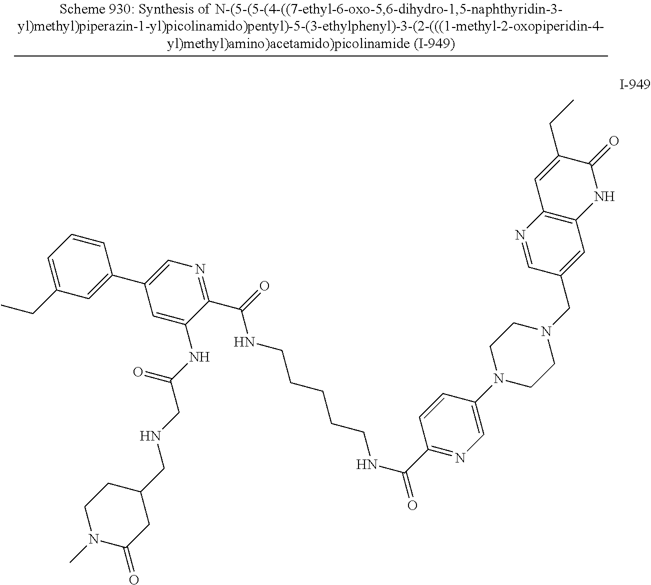 [SMILES: CCc1cccc(-c2cnc(C(=O)NCCCCCNC(=O)c3ccc(N4CCN(Cc5cnc6cc(CC)c(=O)[nH]c6c5)CC4)cn3)c(NC(=O)CNCC3CCN(C)C(=O)C3)c2)c1]

LCMS: Rt=0.452 min, [M+H]⁺=884.6. SFC: Rt=5.732 min, 6.554 min. ¹H NMR (400 MHz, DMSO-d₆) δ=9.31 (d, J=2.0 Hz, 1H), 8.99-8.95 (m, 1H), 8.55 (d, J=2.0 Hz, 1H), 8.41-8.25 (m, 2H), 8.14 (d, J=2.4 Hz, 1H), 7.81-7.73 (m, 2H), 7.63 (s, 1H), 7.53-7.46 (m, 2H), 7.45-7.40 (m, 1H), 7.35-7.26 (m, 2H), 3.61 (s, 2H), 3.32-3.18 (m, 12H), 2.74 (s, 3H), 2.70-2.63 (m, 2H), 2.57-2.51 (m, 4H), 2.49-2.30 (m, 6H), 2.05-1.89 (m, 3H), 1.60-1.28 (m, 7H), 1.22-1.14 (m, 6H)

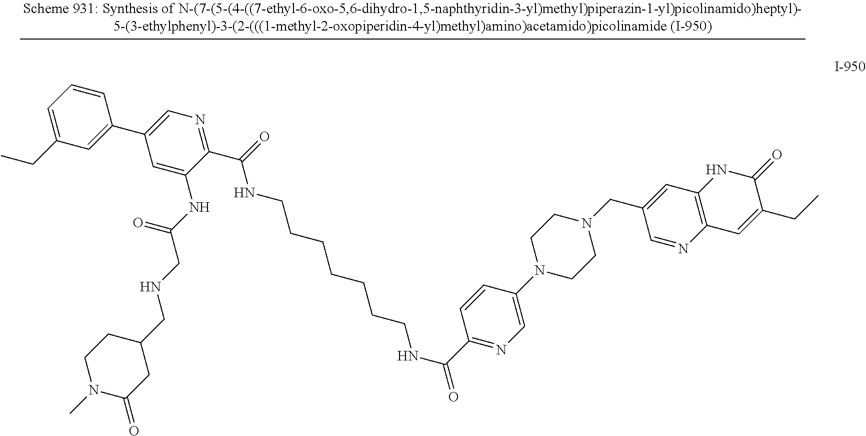 [SMILES: CCc1cccc(-c2cnc(C(=O)NCCCCCCCNC(=O)c3ccc(N4CCN(Cc5cnc6cc(CC)c(=O)[nH]c6c5)CC4)cn3)c(NC(=O)CNCC3CCN(C)C(=O)C3)c2)c1]

LCMS: Rt=0.470 min, [M+H]⁺=912.7. SFC: Rt=5.284 min, 5.894 min. ¹H NMR (400 MHz, DMSO-d₆) δ=9.31 (d, J=2.0 Hz, 1H), 9.00-8.96 (m, 1H), 8.57 (d, J=2.0 Hz, 1H), 8.44-8.26 (m, 2H), 8.21 (d, J=2.4 Hz, 1H), 7.80 (d, J=8.8 Hz, 1H), 7.75 (s, 1H), 7.63 (s, 1H), 7.52-7.47 (m, 2H), 7.46-7.41 (m, 1H), 7.38-7.27 (m, 2H), 3.62 (s, 2H), 3.35-3.17 (m, 12H), 2.75 (s, 3H), 2.70-2.64 (m, 2H), 2.56-2.51 (m, 6H), 2.48-2.28 (m, 4H), 2.08-1.87 (m, 3H), 1.56-1.23 (m, 11H), 1.22-1.14 (m, 6H)

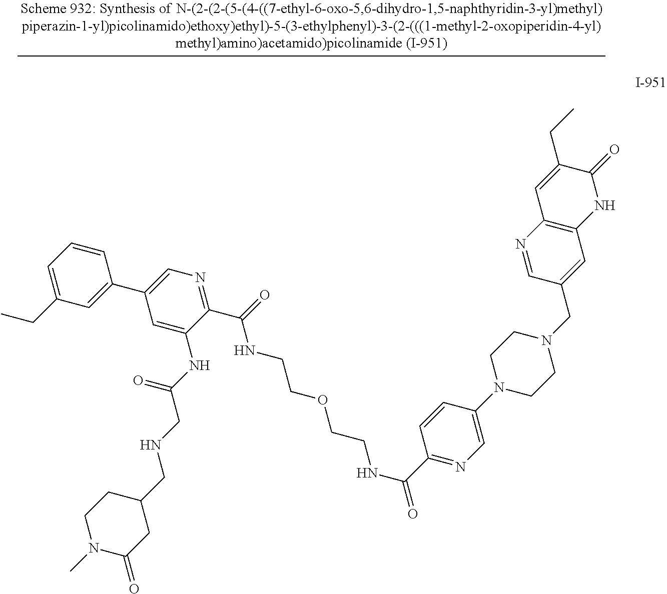 [SMILES: CCc1cccc(-c2cnc(C(=O)NCCOCCNC(=O)c3ccc(N4CCN(Cc5cnc6cc(CC)c(=O)[nH]c6c5)CC4)cn3)c(NC(=O)CNCC3CCN(C)C(=O)C3)c2)c1]

LCMS (Method E): Rt=0.436 min, [M+H]⁺=886.5. SFC: Rt=3.335 min, 3.674 min. ¹H NMR (400 MHz, CHLOROFORM-d) δ=12.75 (s, 1H), 9.41 (d, J=2.0 Hz, 1H), 8.66 (m, 1H), 8.49 (d, J=1.6 Hz, 1H), 8.42 (d, J=2.0 Hz, 1H), 8.15 (m, 1H), 8.01-7.99 (d, J=8.8 Hz, 1H), 7.90-7.81 (m, 2H), 7.70 (d, J=1.2 Hz, 1H), 7.48-7.41 (m, 2H), 7.40-7.33 (m, 1H), 7.25-7.23 (d, J=7.6 Hz, 1H), 7.13 (m, 1H), 3.79-3.60 (m, 11H), 3.53 (d, J=2.4 Hz, 2H), 3.40-3.28 (m, 2H), 3.23-3.12 (m, 4H), 2.95 (s, 3H), 2.81-2.58 (m, 8H), 2.56-2.50 (m, 4H), 2.16-2.05 (m, 3H), 1.59-1.53 (m, 1H), 1.32 (m, 3H), 1.25 (m, 3H).

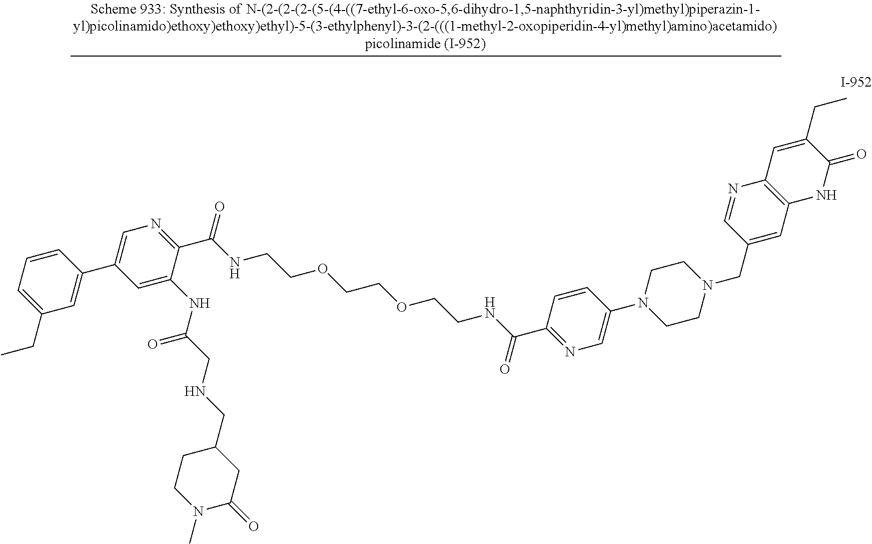 [SMILES: CCc1cccc(-c2cnc(C(=O)NCCOCCOCCNC(=O)c3ccc(N4CCN(Cc5cnc6cc(CC)c(=O)[nH]c6c5)CC4)cn3)c(NC(=O)CNCC3CCN(C)C(=O)C3)c2)c1]

LCMS (Method E): Rt=0.428 min, [M+H]⁺=930.7. SFC: Rt=4.639 min, 5.045 min. ¹H NMR (400 MHz, CHLOROFORM-d) δ=12.75 (s, 1H), 9.39-9.38 (d, J=2.0 Hz, 1H), 8.70 (m, 1H), 8.49-8.48 (d, J=1.6 Hz, 1H), 8.43-8.42 (d, J=2.0 Hz, 1H), 8.18 (m, 1H), 8.08-8.07 (d, J=2.8 Hz, 1H), 8.01-7.99 (d, J=8.8 Hz, 1H), 7.84 (s, 1H), 7.70-7.69 (d, J=1.2 Hz, 1H), 7.46-7.42 (m, 2H), 7.40-7.35 (m, 1H), 7.25 (s, 1H), 7.14 (m, 1H), 3.75-3.64 (m, 14H), 3.53 (d, J=2.4 Hz, 2H), 3.36-3.29 (m, 2H), 3.28-3.20 (m, 4H), 2.95 (s, 3H), 2.81-2.52 (m, 12H), 2.15-2.06 (m, 3H), 1.58 (m, 2H), 1.29 (m, 6H).

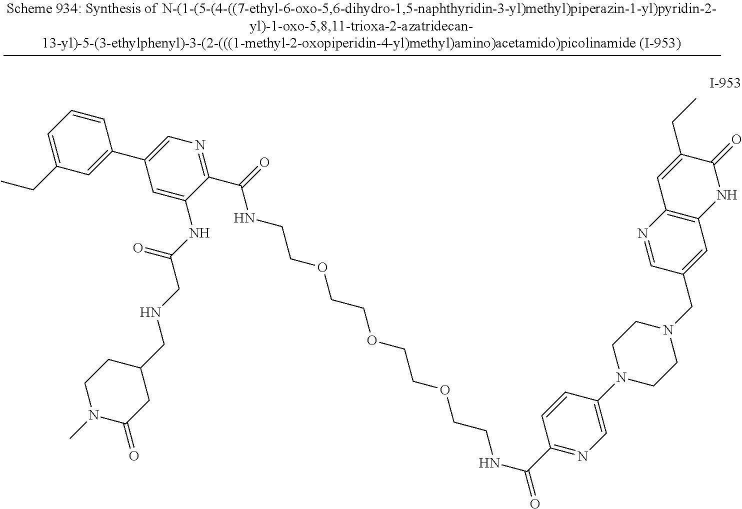 [SMILES: CCc1cccc(-c2cnc(C(=O)NCCOCCOCCOCCNC(=O)c3ccc(N4CCN(Cc5cnc6cc(CC)c(=O)[nH]c6c5)CC4)cn3)c(NC(=O)CNCC3CCN(C)C(=O)C3)c2)c1]

LCMS: Rt=0.634 min, [M+H]⁺=974.7. SFC: Rt=3.749 min, 4.082 min. ¹H NMR (400 MHz, METHANOL-d4) δ=9.44-9.25 (m, 1H), 8.46 (s, 2H), 8.27-8.07 (m, 1H), 7.90-7.68 (m, 3H), 7.54-7.37 (m, 3H), 7.29 (br d, J=8.4 Hz, 2H), 4.60 (br s, 2H), 3.70-3.59 (m, 16H), 3.56-3.37 (m, 8H), 2.93 (s, 3H), 2.60 (br d, J=5.6 Hz, 12H), 2.12 (br s, 3H), 1.28 (q, J=7.6 Hz, 6H)

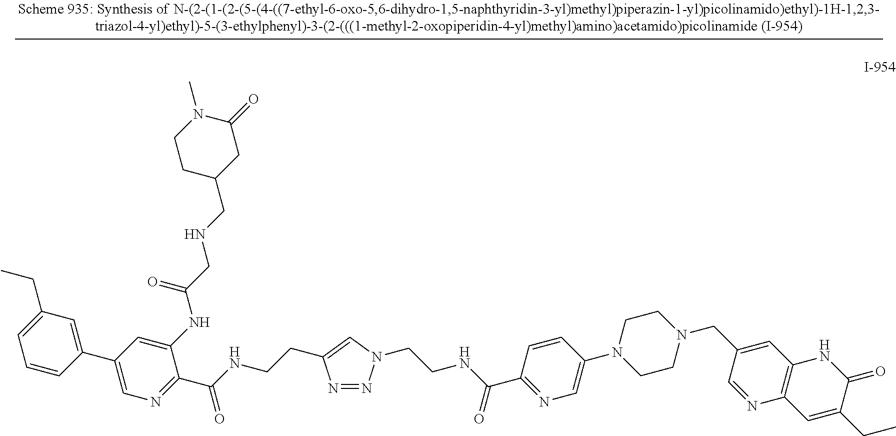 [SMILES: CCc1cccc(-c2cnc(C(=O)NCCc3cn(CCNC(=O)c4ccc(N5CCN(Cc6cnc7cc(CC)c(=O)[nH]c7c6)CC5)cn4)nn3)c(NC(=O)CNCC3CCN(C)C(=O)C3)c2)c1]

LCMS: Rt=0.850 min, [M+H]⁺=937.6. SFC: Rt=7.701 min, 8.367 min. ¹H NMR (400 MHz, METHANOL-d4) δ=9.30 (d, J=1.6 Hz, 1H), 8.45 (s, 2H), 8.05 (d, J=2.4 Hz, 1H), 7.89 (s, 1H), 7.82 (s, 1H), 7.77 (d, J=8.8 Hz, 1H), 7.73 (s, 1H), 7.45 (br d, J=12.8 Hz, 2H), 7.38 (t, J=7.6 Hz, 1H), 7.26 (d, J=7.6 Hz, 1H), 7.19 (dd, J=2.4, 8.8 Hz, 1H), 4.61 (br t, J=5.6 Hz, 2H), 3.85 (br t, J=5.6 Hz, 2H), 3.72-3.63 (m, 4H), 3.44 (d, J=1.6 Hz, 2H), 3.41-3.33 (m, 2H), 3.28-3.20 (m, 4H), 3.02 (t, J=6.8 Hz, 2H), 2.90 (s, 3H), 2.68 (dd, J=7.2, 14.4 Hz, 4H), 2.62-2.50 (m, 7H), 2.20-2.03 (m, 3H), 1.63-1.51 (m, 1H), 1.27 (td, J=7.6, 10.4 Hz, 6H)

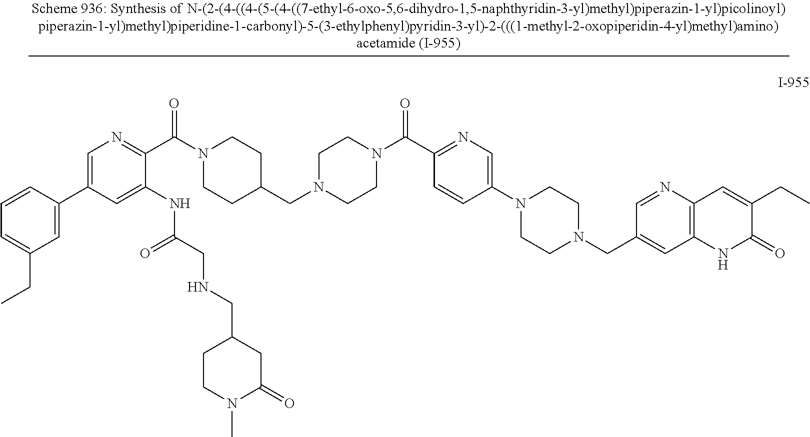 [SMILES: CCc1cccc(-c2cnc(C(=O)N3CCC(CN4CCN(C(=O)c5ccc(N6CCN(Cc7cnc8cc(CC)c(=O)[nH]c8c7)CC6)cn5)CC4)CC3)c(NC(=O)CNCC3CCN(C)C(=O)C3)c2)c1]

LCMS: Rt=0.850 min, [M+H]⁺=965.7. SFC: Rt=7.688 min, 8.699 min. ¹H NMR (400 MHz, METHANOL-d4) δ=8.85 (dd, J=2.0, 6.0 Hz, 1H), 8.59 (d, J=1.6 Hz, 1H), 8.49 (d, J=1.6 Hz, 1H), 8.24 (d, J=2.8 Hz, 1H), 7.83 (s, 1H), 7.76 (s, 1H), 7.57-7.47 (m, 3H), 7.46-7.38 (m, 2H), 7.31 (d, J=7.6 Hz, 1H), 4.69 (br d, J=12.4 Hz, 1H), 3.84-3.76 (m, 2H), 3.74 (s, 2H), 3.62 (br s, 2H), 3.43-3.35 (m, 8H), 3.15 (br t, J=12.8 Hz, 1H), 2.94 (s, 4H), 2.74 (q, J=7.6 Hz, 3H), 2.68 (br t, J=6.8 Hz, 6H), 2.64-2.58 (m, 2H), 2.57-2.49 (m, 3H), 2.44 (br s, 2H), 2.28 (br d, J=6.4 Hz, 2H), 2.19-2.06 (m, 3H), 2.02-1.88 (m, 2H), 1.79 (br d, J=13.2 Hz, 1H), 1.66-1.54 (m, 1H), 1.37-1.15 (m, 9H)

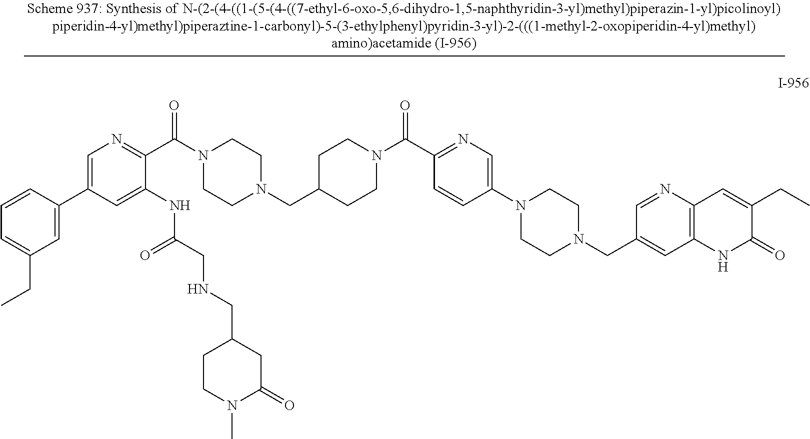 [SMILES: CCc1cccc(-c2cnc(C(=O)N3CCN(CC4CCN(C(=O)c5ccc(N6CCN(Cc7cnc8cc(CC)c(=O)[nH]c8c7)CC6)cn5)CC4)CC3)c(NC(=O)CNCC3CCN(C)C(=O)C3)c2)c1]

LCMS: Rt=0.394 min, [M+H]⁺=965.9. SFC: Rt=8.684 min, 13.134 min. ¹H NMR (400 MHz, CHLOROFORM-d) δ=11.36-11.13 (m, 1H), 10.76 (s, 1H), 9.06 (d, J=2.0 Hz, 1H), 8.59-8.45 (m, 2H), 8.23 (d, J=2.8 Hz, 1H), 7.88 (s, 1H), 7.67 (s, 1H), 7.60 (d, J=8.8 Hz, 1H), 7.47 (td, J=1.5, 3.2 Hz, 2H), 7.44-7.39 (m, 1H), 7.30 (s, 1H), 7.22 (dd, J=2.9, 8.8 Hz, 1H), 4.80-4.68 (m, 1H), 4.26-4.14 (m, 1H), 3.86 (br s, 2H), 3.72 (s, 4H), 3.55-3.42 (m, 2H), 3.40-3.29 (m, 6H), 3.16-3.02 (m, 1H), 2.97 (s, 3H), 2.80-2.71 (m, 5H), 2.71-2.63 (m, 6H), 2.61 (br s, 1H), 2.57 (br d, J=7.8 Hz, 2H), 2.50 (br s, 2H), 2.28 (br d, J=2.0 Hz, 2H), 2.18-2.08 (m, 3H), 1.95-1.87 (m, 2H), 1.68-1.55 (m, 2H), 1.40-1.15 (m, 9H)1

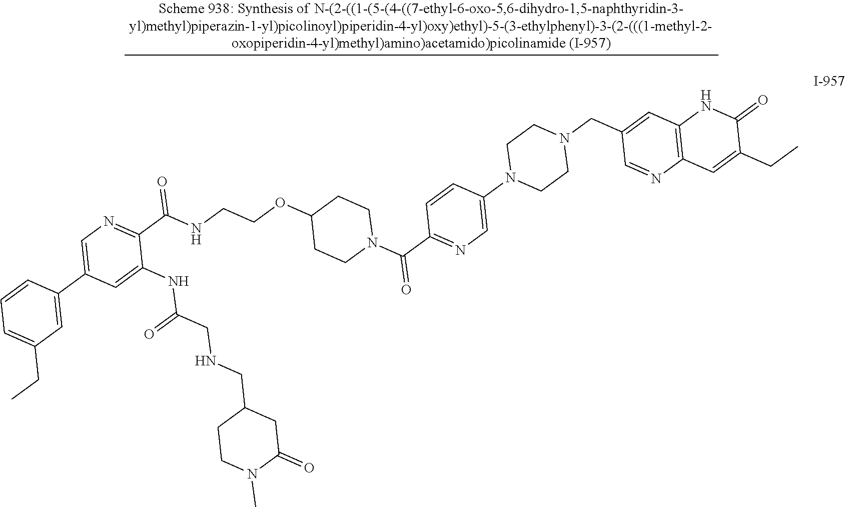 [SMILES: CCc1cccc(-c2cnc(C(=O)NCCOC3CCN(C(=O)c4ccc(N5CCN(Cc6cnc7cc(CC)c(=O)[nH]c7c6)CC5)cn4)CC3)c(NC(=O)CNCC3CCN(C)C(=O)C3)c2)c1]

LCMS: Rt=0.644 min, [M+H]⁺=926.6. SFC: Rt=6.276 min, 6.887 min. ¹H NMR (400 MHz, METHANOL-d4) δ=9.38 (d, J=2.0 Hz, 1H), 8.60 (d, J=2.0 Hz, 1H), 8.50 (d, J=1.6 Hz, 1H), 8.18 (d, J=2.4 Hz, 1H), 7.84 (s, 1H), 7.77 (d, J=1.2 Hz, 1H), 7.57-7.50 (m, 2H), 7.44 (s, 3H), 7.34-7.28 (m, 1H), 4.60 (br s, 3H), 4.03-3.86 (m, 1H), 3.79-3.70 (m, 6H), 3.63 (br d, J=5.2 Hz, 4H), 3.51-3.40 (m, 4H), 2.93 (s, 3H), 2.78-2.54 (m, 12H), 2.20-2.10 (m, 3H), 2.01-1.73 (m, 3H), 1.67-1.59 (m, 2H), 1.30-1.25 (m, 6H)

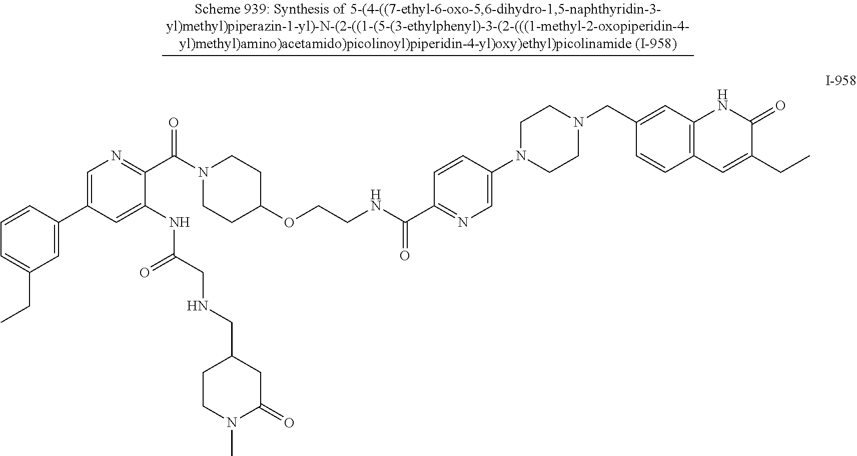 [SMILES: CCc1cccc(-c2cnc(C(=O)N3CCC(OCCNC(=O)c4ccc(N5CCN(Cc6ccc7cc(CC)c(=O)[nH]c7c6)CC5)cn4)CC3)c(NC(=O)CNCC3CCN(C)C(=O)C3)c2)c1]

LCMS: Rt=0.854 min, [M+H]⁺=926.6. SFC: Rt=4.719 min, 6.121 min. ¹H NMR (400 MHz, CHLOROFORM-d) δ=10.68 (d, J=6.6 Hz, 1H), 9.04 (t, J=2.2 Hz, 1H), 8.51 (dd, J=1.8, 6.6 Hz, 2H), 8.23-8.09 (m, 2H), 8.05 (d, J=8.8 Hz, 1H), 7.85 (s, 1H), 7.66 (s, 1H), 7.45 (br d, J=5.4 Hz, 2H), 7.43-7.37 (m, 1H), 7.21 (dd, J=2.9, 8.8 Hz, 1H), 4.09-3.97 (m, 1H), 3.87-3.77 (m, 1H), 3.74-3.59 (m, 8H), 3.54-3.41 (m, 3H), 3.39-3.27 (m, 6H), 2.95 (s, 3H), 2.78-2.52 (m, 11H), 2.18-2.04 (m, 3H), 2.03-1.90 (m, 2H), 1.84 (br s, 2H), 1.64-1.53 (m, 2H), 1.30 (td, J=7.5, 9.6 Hz, 6H)3

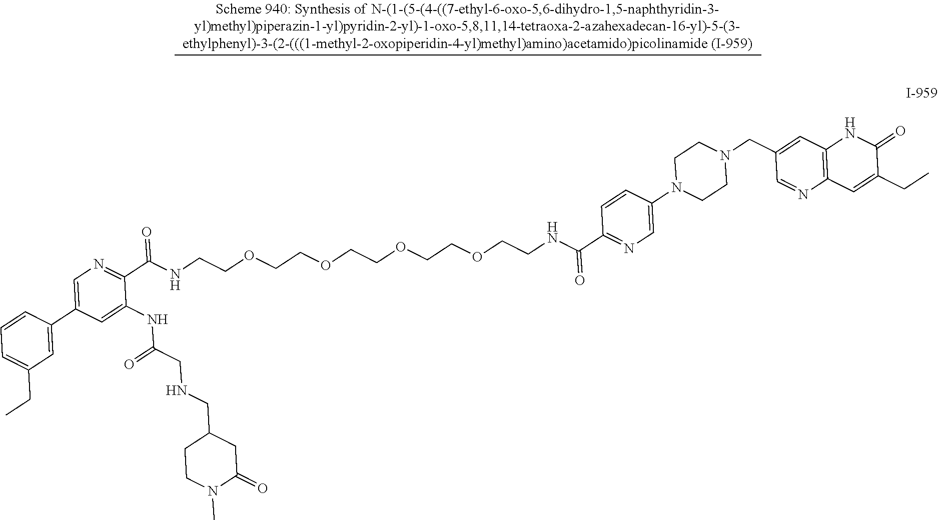 [SMILES: CCc1cccc(-c2cnc(C(=O)NCCOCCOCCOCCOCCNC(=O)c3ccc(N4CCN(Cc5cnc6cc(CC)c(=O)[nH]c6c5)CC4)cn3)c(NC(=O)CNCC3CCN(C)C(=O)C3)c2)c1]

LCMS: Rt=0.636 min, [M+H]⁺=1018.7. SFC: Rt=3.848 min, 4.190 min. ¹H NMR (400 MHz, CHLOROFORM-d) δ=12.75 (s, 1H), 11.40-11.12 (m, 1H), 9.41 (d, J=2.0 Hz, 1H), 8.66 (m, 1H), 8.48 (dd, J=2.0, 16.4 Hz, 2H), 8.16-8.09 (m, 2H), 8.02 (d, J=8.8 Hz, 1H), 7.84 (s, 1H), 7.67 (s, 1H), 7.48-7.43 (m, 2H), 7.39 (t, J=8.0 Hz, 1H), 7.28 (br s, 1H), 7.18 (dd, J=2.8, 8.8 Hz, 1H), 3.70-3.64 (m, 22H), 3.52 (d, J=2.4 Hz, 2H), 3.35-3.29 (m, 6H), 2.95 (s, 3H), 2.78-2.57 (m, 12H), 2.16-2.06 (m, 3H), 1.57 (ddd, J=2.8, 7.2, 10.0 Hz, 1H), 1.33-1.30 (m, 3H), 1.29-1.26 (m, 3H)

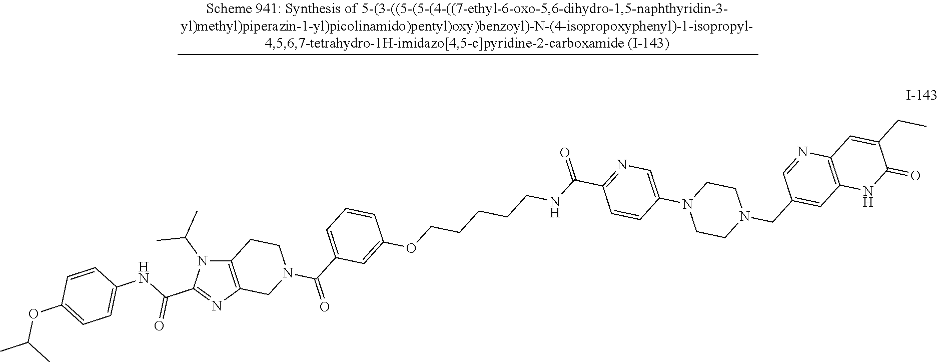 [SMILES: CCc1cc2ncc(CN3CCN(c4ccc(C(=O)NCCCCCOc5cccc(C(=O)N6CCc7c(nc(C(=O)Nc8ccc(OC(C)C)cc8)n7C(C)C)C6)c5)nc4)CC3)cc2[nH]c1=O]

LCMS (Method E): Rt=0.552 min, [M+H]⁺=923.6. ¹H NMR (400 MHz, DMSO-d6) δ=11.85 (s, 1H), 10.29-9.90 (m, 1H), 8.49-8.34 (m, 2H), 8.25 (d, J=1.9 Hz, 1H), 7.81 (d, J=8.8 Hz, 1H), 7.75 (s, 1H), 7.72-7.54 (m, 3H), 7.43-7.28 (m, 2H), 7.08-6.92 (m, 3H), 6.92-6.77 (m, 2H), 5.73-5.47 (m, 1H), 4.68-4.32 (m, 3H), 4.10-3.86 (m, 3H), 3.71-3.53 (m, 3H), 3.33-3.31 (m, 6H), 2.99-2.84 (m, 2H), 2.58-2.52 (m, 6H), 1.80-1.68 (m, 2H), 1.64-1.51 (m, 2H), 1.49-1.37 (m, 8H), 1.28-1.20 (m, 6H), 1.20-1.15 (m, 3H)

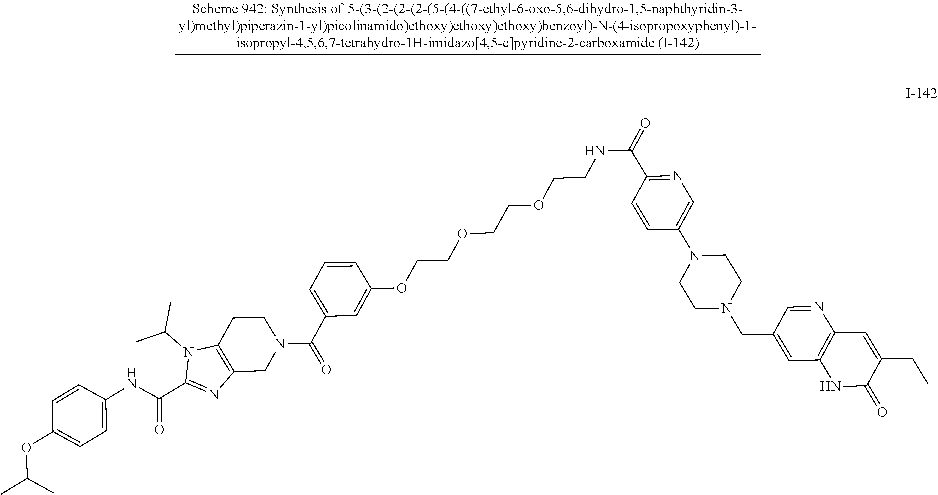 [SMILES: CCc1cc2ncc(CN3CCN(c4ccc(C(=O)NCCOCCOCCOc5cccc(C(=O)N6CCc7c(nc(C(=O)Nc8ccc(OC(C)C)cc8)n7C(C)C)C6)c5)nc4)CC3)cc2[nH]c1=O]

LCMS: Rt=0.680 min, [M+H]⁺=969.6. ¹H NMR (400 MHz, DMSO-d6) δ=11.85 (br s, 1H), 10.31-9.86 (m, 1H), 8.40 (d, J=1.6 Hz, 1H), 8.34 (t, J=6.0 Hz, 1H), 8.24-8.23 (m, 1H), 7.82-7.80 (m, 1H), 7.75 (s, 1H), 7.71-7.55 (m, 3H), 7.42-7.31 (m, 2H), 7.07-6.94 (m, 3H), 6.85 (br s, 2H), 5.62-5.61 (m, 1H), 4.67-4.30 (m, 3H), 4.11 (br s, 2H), 3.99-3.87 (m, 1H), 3.75 (br s, 2H), 3.65-3.51 (m, 9H), 3.46-3.41 (m, 2H), 3.32-3.28 (m, 4H), 2.90 (br s, 2H), 2.57-2.51 (m, 6H), 1.44-1.43 (m, 6H), 1.23-1.22 (m, 6H), 1.18-1.15 (m, 3H)

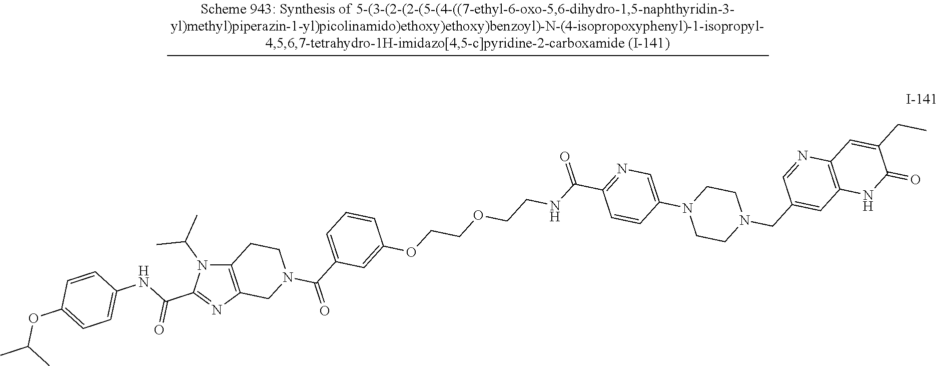 [SMILES: CCc1cc2ncc(CN3CCN(c4ccc(C(=O)NCCOCCOc5cccc(C(=O)N6CCc7c(nc(C(=O)Nc8ccc(OC(C)C)cc8)n7C(C)C)C6)c5)nc4)CC3)cc2[nH]c1=O]

LCMS: Rt=0.509 min, [M+H]⁺=925.8. ¹H NMR (400 MHz, DMSO-d6) δ=11.95-11.71 (m, 1H), 10.39-9.97 (m, 1H), 8.42-8.32 (m, 2H), 8.23 (d, J=2.4 Hz, 1H), 7.82 (d, J=8.8 Hz, 1H), 7.75 (s, 1H), 7.71-7.58 (m, 3H), 7.42-7.32 (m, 2H), 7.09-6.97 (m, 3H), 6.85 (d, J=6.0 Hz, 2H), 5.69-5.53 (m, 1H), 4.66-4.51 (m, 2H), 4.46-4.35 (m, 1H), 4.14 (br s, 2H), 4.00-3.86 (m, 1H), 3.77 (br s, 2H), 3.66-3.56 (m, 5H), 3.49-3.43 (m, 2H), 3.31-3.27 (m, 4H), 2.97-2.82 (m, 2H), 2.57-2.52 (m, 6H), 1.44 (d, J=6.8 Hz, 6H), 1.23 (br s, 6H), 1.18 (t, J=7.2 Hz, 3H)

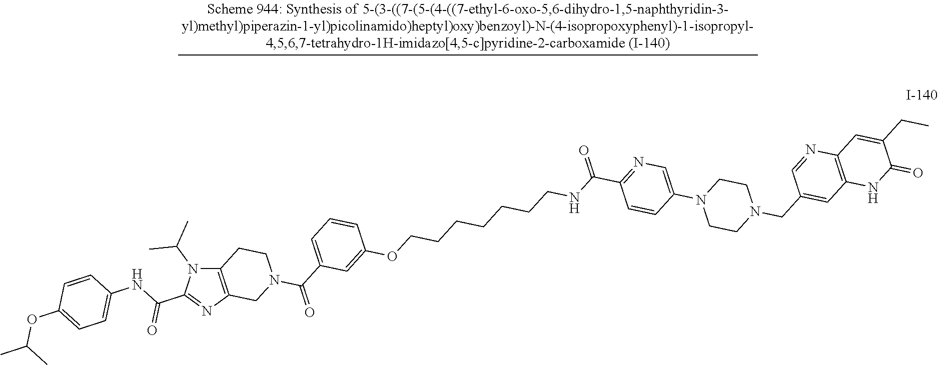 [SMILES: CCc1cc2ncc(CN3CCN(c4ccc(C(=O)NCCCCCCCOc5cccc(C(=O)N6CCc7c(nc(C(=O)Nc8ccc(OC(C)C)cc8)n7C(C)C)C6)c5)nc4)CC3)cc2[nH]c1=O]

LCMS: Retention time: 0.562 min, [M+H]⁺=951.6. ¹H NMR (400 MHz, DMSO-d6) δ=11.85 (s, 1H), 10.36-9.86 (m, 1H), 8.45-8.33 (m, 2H), 8.25 (d, J=2.4 Hz, 1H), 7.81 (d, J=8.8 Hz, 1H), 7.76-7.59 (m, 4H), 7.40-7.32 (m, 2H), 7.06-6.93 (m, 3H), 6.84 (br s, 2H), 5.62 (d, J=1.4 Hz, 1H), 4.69-4.35 (m, 3H), 3.98 (br s, 3H), 3.67-3.54 (m, 3H), 3.25 (d, J=6.4 Hz, 2H), 2.91 (br s, 2H), 2.59-2.51 (m, 8H), 1.69 (d, J=5.6 Hz, 2H), 1.55-1.27 (m, 15H), 1.26-1.15 (m, 10H)

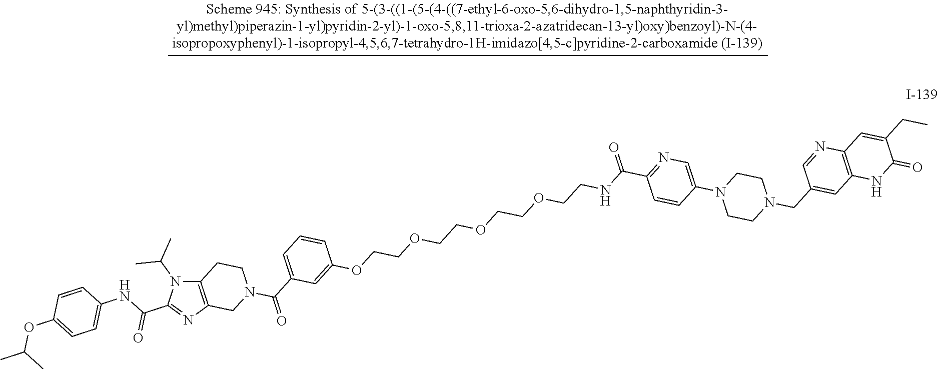 [SMILES: CCc1cc2ncc(CN3CCN(c4ccc(C(=O)NCCOCCOCCOCCOc5cccc(C(=O)N6CCc7c(nc(C(=O)Nc8ccc(OC(C)C)cc8)n7C(C)C)C6)c5)nc4)CC3)cc2[nH]c1=O]

LCMS: Rt=0.512 min, [M+H]⁺=1035.6. ¹H NMR (400 MHz, DMSO-d6) δ=11.92-11.77 (m, 1H), 10.28-9.95 (m, 1H), 8.39 (d, J=2.0 Hz, 1H), 8.33 (t, J=6.0 Hz, 1H), 8.26 (d, J=2.4 Hz, 1H), 7.82 (d, J=8.8 Hz, 1H), 7.75 (s, 1H), 7.72-7.56 (m, 3H), 7.43-7.30 (m, 2H), 7.09-6.94 (m, 3H), 6.93-6.74 (m, 2H), 5.70-5.51 (m, 1H), 4.73-4.33 (m, 3H), 4.11 (br s, 2H), 4.00-3.84 (m, 1H), 3.73 (br s, 2H), 3.66-3.61 (m, 2H), 3.60-3.48 (m, 11H), 3.42 (q, J=6.0 Hz, 2H), 3.33-3.32 (m, 4H), 3.01-2.84 (m, 2H), 2.58-2.52 (m, 6H), 1.45 (d, J=6.8 Hz, 6H), 1.31-1.13 (m, 9H). ¹H NMR (400 MHz, DMSO-d6+D2O) δ=8.42-8.29 (m, 2H), 8.23 (d, J=2.8 Hz, 1H), 7.80 (d, J=8.8 Hz, 1H), 7.74 (s, 1H), 7.69-7.51 (m, 3H), 7.41-7.31 (m, 2H), 7.07-6.92 (m, 3H), 6.91-6.76 (m, 2H), 5.64-5.51 (m, 1H), 4.67-4.33 (m, 3H), 4.14-4.03 (m, 2H), 3.99-3.83 (m, 1H), 3.71 (br s, 2H), 3.63-3.60 (m, 4H), 3.54-3.48 (m, 9H), 3.44-3.37 (m, 2H), 3.30 (br s, 4H), 2.97-2.83 (m, 2H), 2.53 (br s, 6H), 1.42 (d, J=6.8 Hz, 6H), 1.28-1.12 (m, 9H)

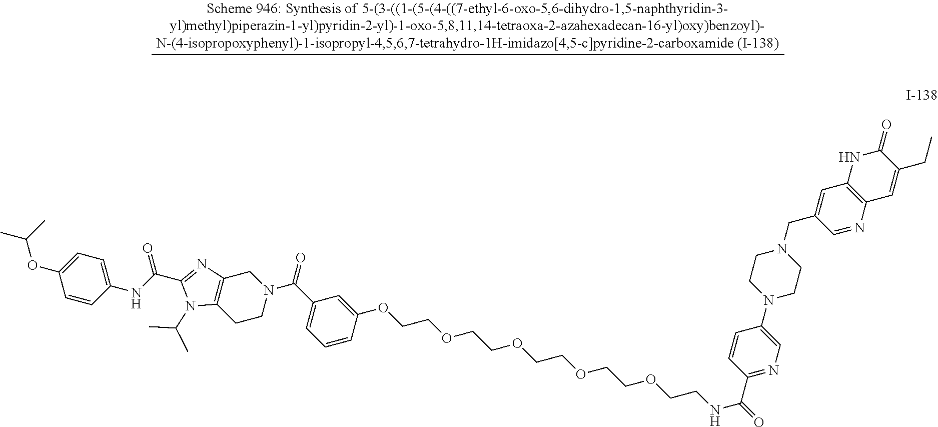 [SMILES: CCc1cc2ncc(CN3CCN(c4ccc(C(=O)NCCOCCOCCOCCOCCOc5cccc(C(=O)N6CCc7c(nc(C(=O)Nc8ccc(OC(C)C)cc8)n7C(C)C)C6)c5)nc4)CC3)cc2[nH]c1=O]

LCMS: Rt=0.526 min, [M+H]⁺=1057.6. ¹H NMR (400 MHz, DMSO-d6) δ=12.01-11.75 (m, 1H), 10.28-9.99 (m, 1H), 8.39 (d, J=1.6 Hz, 1H), 8.34-8.31 (m, 1H), 8.27 (d, J=2.8 Hz, 1H), 7.82 (d, J=8.8 Hz, 1H), 7.75 (s, 1H), 7.72-7.56 (m, 3H), 7.43-7.33 (m, 2H), 7.09-6.95 (m, 3H), 6.92-6.76 (m, 2H), 5.71-5.52 (m, 1H), 4.71-4.27 (m, 3H), 4.19-3.86 (m, 3H), 3.76-3.70 (m, 2H), 3.63 (s, 2H), 3.59-3.46 (m, 15H), 3.44-3.39 (m, 2H), 3.36-3.32 (m, 4H), 2.98-2.84 (m, 2H), 2.57-2.52 (m, 6H), 1.45 (d, J=7.2 Hz, 6H), 1.23 (br d, J=4.8 Hz, 6H), 1.20-1.16 (m, 3H)

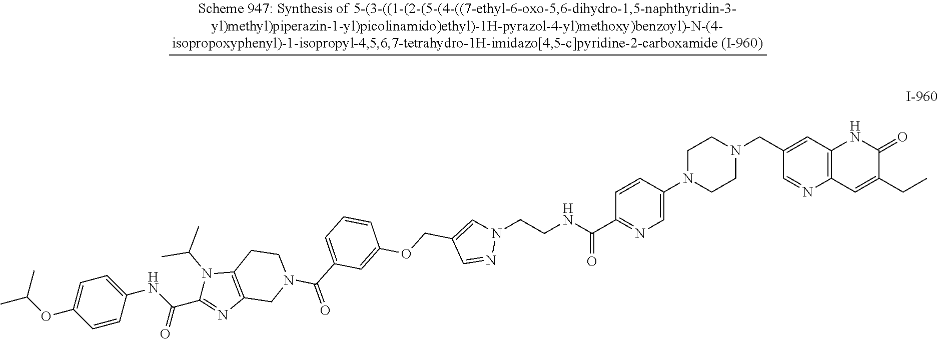 [SMILES: CCc1cc2ncc(CN3CCN(c4ccc(C(=O)NCCn5cc(COc6cccc(C(=O)N7CCc8c(nc(C(=O)Nc9ccc(OC(C)C)cc9)n8C(C)C)C7)c6)cn5)nc4)CC3)cc2[nH]c1=O]

LCMS: Rt=0.639 min, [M+H]⁺=961.6. ¹H NMR (400 MHz, DMSO-d6) δ=12.18 (s, 1H), 10.32-9.97 (m, 1H), 9.85 (br s, 1H), 8.68-8.53 (m, 2H), 8.33 (d, J=2.0 Hz, 1H), 7.90-7.77 (m, 3H), 7.65 (br s, 2H), 7.52-7.43 (m, 2H), 7.31 (s, 1H), 7.22-7.00 (m, 2H), 6.94-6.74 (m, 4H), 5.74-5.53 (m, 1H), 4.62-4.36 (m, 5H), 4.32-3.90 (m, 5H), 3.84-3.56 (m, 8H), 3.02-2.82 (m, 3H), 2.61-2.54 (m, 3H), 1.46 (d, J=6.8. Hz, 6H), 1.26-1.19 (m, 9H)

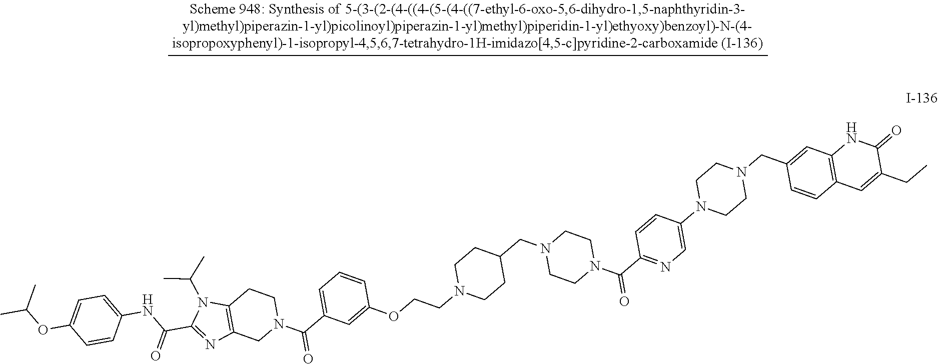 [SMILES: CCc1cc2ccc(CN3CCN(c4ccc(C(=O)N5CCN(CC6CCN(CCOc7cccc(C(=O)N8CCc9c(nc(C(=O)Nc%10ccc(OC(C)C)cc%10)n9C(C)C)C8)c7)CC6)CC5)nc4)CC3)cc2[nH]c1=O]

LCMS: Rt=0.706 min, [M+H]⁺=1047.7. ¹HNMR (400 MHz, DMSO-d6) δ=11.85 (s, 1H), 10.29-9.94 (m, 1H), 8.40 (d, J=1.6 Hz, 1H), 8.24 (d, J=2.4 Hz, 1H), 7.78-7.57 (m, 4H), 7.46-7.31 (m, 3H), 7.09-6.94 (m, 3H), 6.85 (d, J=1.2 Hz, 2H), 5.74-5.50 (m, 1H), 4.75-4.32 (m, 4H), 4.21-3.87 (m, 4H), 3.70-3.53 (m, 3H), 3.30 (d, J=4.4 Hz, 6H), 3.04-2.88 (m, 3H), 2.76-2.64 (m, 3H), 2.57-2.51 (m, 8H), 2.33 (d, J=1.8 Hz, 4H), 2.10 (br s, 2H), 1.83-1.55 (m, 3H), 1.46 (d, J=7.2 Hz, 6H), 1.26-1.15 (m, 9H), 1.09-0.96 (m, 2H)

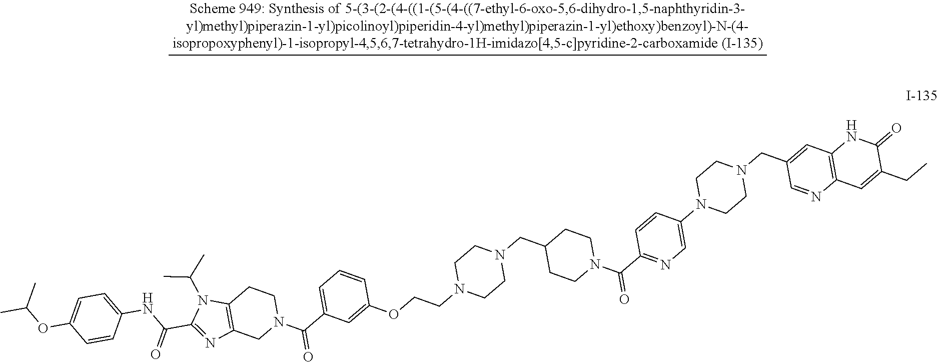 [SMILES: CCc1cc2ncc(CN3CCN(c4ccc(C(=O)N5CCC(CN6CCN(CCOc7cccc(C(=O)N8CCc9c(nc(C(=O)Nc%10ccc(OC(C)C)cc%10)n9C(C)C)C8)c7)CC6)CC5)nc4)CC3)cc2[nH]c1=O]

LCMS: Retention time: 0.706 min, [M+H]⁺=1047.7. ¹H NMR (400 MHz, DMSO-d6) δ=11.85 (s, 1H), 10.29-9.94 (m, 1H), 8.40 (d, J=1.6 Hz, 1H), 8.24 (d, J=2.4 Hz, 1H), 7.78-7.57 (m, 4H), 7.46-7.31 (m, 3H), 7.09-6.94 (m, 3H), 6.85 (d, J=1.2 Hz, 2H), 5.74-5.50 (m, 1H), 4.75-4.32 (m, 4H), 4.21-3.87 (m, 4H), 3.70-3.53 (m, 3H), 3.30 (d, J=4.4 Hz, 6H), 3.04-2.88 (m, 3H), 2.76-2.64 (m, 3H), 2.57-2.51 (m, 8H), 2.33 (d, J=1.8 Hz, 4H), 2.10 (br s, 2H), 1.83-1.55 (m, 3H), 1.46 (d, J=7.2 Hz, 6H), 1.26-1.15 (m, 9H), 1.09-0.96 (in, 2H)

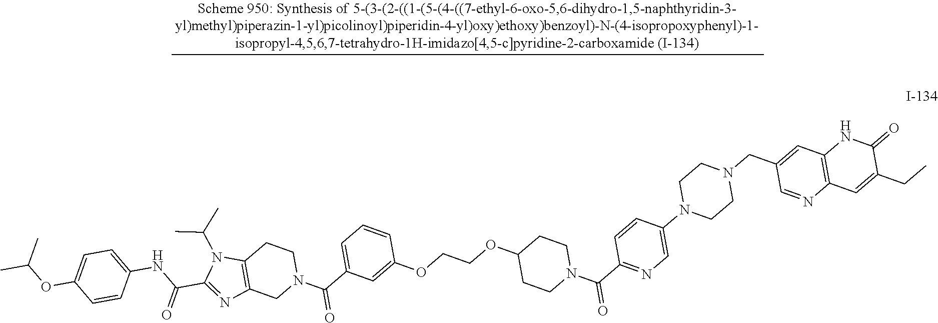 [SMILES: CCc1cc2ncc(CN3CCN(c4ccc(C(=O)N5CCC(OCCOc6cccc(C(=O)N7CCc8c(nc(C(=O)Nc9ccc(OC(C)C)cc9)n8C(C)C)C7)c6)CC5)nc4)CC3)cc2[nH]c1=O]

LCMS: Rt=0.512 min, [M+H]⁺=965.5. ¹H NMR (400 MHz, DMSO-d6) δ=8.39 (d, J=2.0 Hz, 1H), 8.20 (d, J=2.4 Hz, 1H), 7.75 (s, 1H), 7.68-7.52 (m, 3H), 7.45-7.30 (m, 3H), 7.08-6.94 (m, 3H), 6.84 (br s, 2H), 5.70-5.48 (m, 1H), 4.68-4.45 (m, 2H), 4.39 (br s, 1H), 4.12 (br s, 2H), 3.93 (br s, 2H), 3.76 (br s, 3H), 3.62 (br s, 4H), 3.26 (br s, 6H), 2.98-2.83 (m, 2H), 2.58-2.51 (m, 6H), 1.95-1.74 (m, 2H), 1.52-1.36 (m, 8H), 1.29-1.10 (m, 9H)

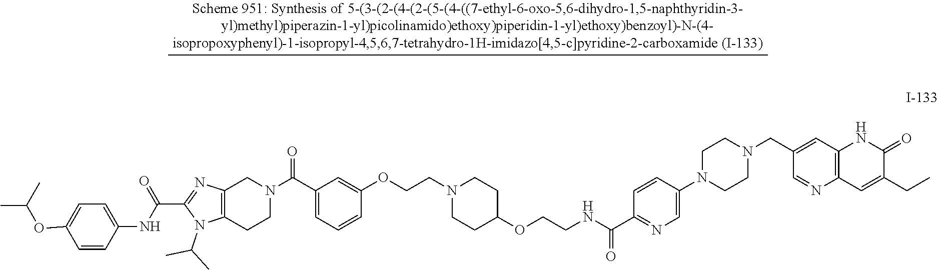 [SMILES: CCc1cc2ncc(CN3CCN(c4ccc(C(=O)NCCOC5CCN(CCOc6cccc(C(=O)N7CCc8c(nc(C(=O)Nc9ccc(OC(C)C)cc9)n8C(C)C)C7)c6)CC5)nc4)CC3)cc2[nH]c1=O]

LCMS: Rt=0.475 min, [M+H]⁺=1008.6. HNMR (400 MHz, DMSO-d6) δ=11.84 (s, 1H), 10.26-9.96 (m, 1H), 8.40 (s, 1H), 8.32-8.30 (m, 1H), 8.27 (d, J=2.4 Hz, 1H), 7.83 (d, J=8.8 Hz, 1H), 7.75 (s, 1H), 7.71-7.58 (m, 3H), 7.48-7.25 (m, 2H), 7.13-6.93 (m, 3H), 6.91-6.76 (m, 2H), 5.63-5.62 (m, 1H), 4.69-4.35 (m, 3H), 4.18-3.85 (m, 3H), 3.67-3.56 (m, 3H), 3.54-3.47 (m, 2H), 3.44-3.33 (m, 7H), 2.91-2.90 (m, 2H), 2.82-2.71 (m, 2H), 2.66 (br s, 2H), 2.54-2.50 (m, 6H), 2.16 (t, J=9.6 Hz, 2H), 1.86-1.75 (m, 2H), 1.48-1.37 (m, 8H), 1.24-1.23 (m, 6H), 1.18 (t, J=7.2 Hz, 3H)

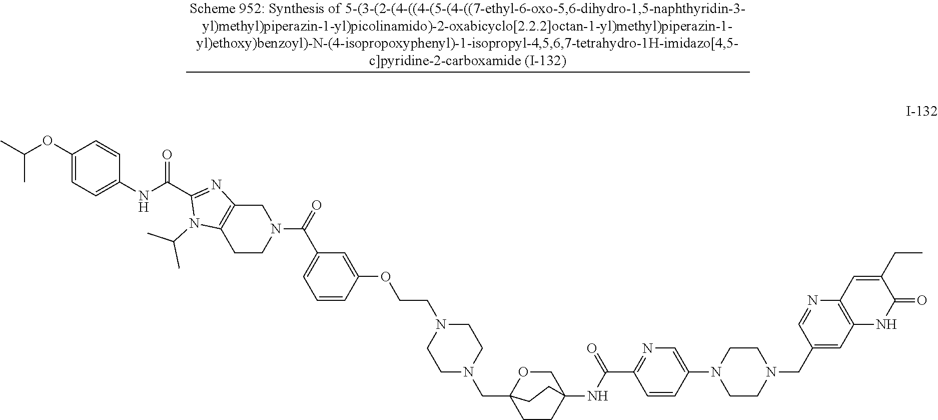 [SMILES: CCc1cc2ncc(CN3CCN(c4ccc(C(=O)NC56CCC(CN7CCN(CCOc8cccc(C(=O)N9CCc%10c(nc(C(=O)Nc%11ccc(OC(C)C)cc%11)n%10C(C)C)C9)c8)CC7)(CC5)OC6)nc4)CC3)cc2[nH]c1=O]

LCMS: Rt=0.484 min, [M+H]⁺=1089.6. ¹H NMR (400 MHz, METHANOL-d₄) δ=8.49 (d, J=2.0 Hz, 1H), 8.23 (d, J=2.8 Hz, 1H), 7.89-7.80 (m, 2H), 7.76 (d, J=1.2 Hz, 1H), 7.63-7.48 (m, 2H), 7.44-7.41 (m, 1H), 7.36-7.33 (m, 1H), 7.18-7.01 (m, 3H), 6.88 (br s, 2H), 5.72 (br s, 1H), 4.73 (br s, 1H), 4.62-4.45 (m, 2H), 4.26 (br s, 2H), 4.09 (br s, 3H), 3.80-3.68 (m, 3H), 3.43-3.36 (m, 4H), 3.15-2.77 (m, 12H), 2.71-2.62 (m, 6H), 2.56-2.43 (m, 2H), 2.33-2.21 (m, 2H), 2.13-1.96 (m, 4H), 1.82-1.66 (m, 2H), 1.54 (d, J=7.2 Hz, 6H), 1.34-1.24 (m, 9H)

### Example 2: Biological Assays

### PARP Inhibitors, Cell Lines, and Cell Culture

All cell lines were obtained from ATCC, Horizon Discovery, or BioIVT or other sources.

### Colony Formation Assay

Cells are seeded in 24-well plates (500-1,500 cells/well, depending on the doubling times) and incubated overnight. PARPi are dispensed in titration dilutions, covering 0.1 nmol/L-40 mol/L; each concentration is tested in triplicate and DMSO is used as untreated control. Plates are incubated at 37° C., 5% CO2 for 8-13 days, to allow colony formation. Cells are then fixed and stained with Blue-G-250 brilliant blue [#B8522-1EA, Sigma, reconstituted in 25% (v/v) methanol and 5% (v/v) acetic acid] for 15 min then thoroughly washed with dH2O. Colonies are analyzed by total optical density measured with software. Data analysis is performed by normalization on the vehicle treated of the respective plate set as 1. Data are plotted and IC50s are calculated with GraphPad Prism software.

### PARylation Inhibition Assay

Cells were seeded into 384-well plates and incubated overnight at 37° C. and 5% CO₂ in a rotating incubator. PARPi compounds were added with a final compound concentration range between 1.6 μmol/L and 30 μmol/L. After 1 hour incubation with PARPi, H₂O₂ was added at final concentration of 10 mmol/L and incubated for 5 min in culture conditions. Cells were then fixed in ice-cold methanol for 15 minutes at 4° C. and processed for immunostaining with primary antibody Poly/Mono-ADP Ribose (Cell Signaling Technology, #83732) followed by secondary goat anti-rabbit AlexaFluor-488 antibody (Invitrogen, #A-11008); nuclei were counterstained with DAPI. The nuclear PAR fluorescence intensity (F.I.) was acquired and analyzed; dose-response curves were generated with GraphPad Prism software.

### PARP Trapping Assay

Cells were plated at a density of 6,000 cells per well. PARPi were added at final concentration range between 1.6 μmol/L and 30 mol/L and incubated for 4 hours; Methyl methanesulfonate (MMS; Sigma Aldrich, 129925) was added at final 0.01%. Cell media was removed and a pre-extraction step was performed for 10 min at 4° C. with cold cytoskeleton (CSK) buffer (10 mmol/L PIPES pH=6.8, 300 mmol/L sucrose, 200 mmol/L NaCl, 3 mmol/L MgCl2) supplemented with 0.6% Triton X-100. Cells were then fixed with ice-cold methanol for 15 minutes at −20° C.; blocking solution (PBS+0.1% Tween20+3% BSA) was added and incubated for 1 hour at room temperature. Immunostaining of PARP1 or PARP2 was performed by adding the primary antibody (PARP1 antibody, ProteinTech, 13371-1-AP; PARP2 antibody, Active Motif, 39743) diluted 1:1,000 in PBS+1% BSA and incubated overnight at 4° C., followed by incubation with secondary goat anti-rabbit AlexaFluor 488 antibody for 1 hour at room temperature. DNA was counterstained with Hoechst 33342 (Invitrogen, H3570) diluted 1:5,000 in PBS+1% BSA. The nuclear F.I. was acquired and analyzed; dose-response curves were generated with GraphPad Prism Software.

### DNA Damage and Cell-Cycle Analysis by Immunofluorescence and Flow Cytometry

Cells are seeded in 96-well plates for immunofluorescence and in 12-well plates for flow cytometry analysis. Compounds are added from compound stocks dissolved in DMSO, in duplicate for each condition. Plates are incubated for 24, 48, or 72 hours. During the last 30 minutes of incubation, EdU is added to cells, at final concentration of 10 μmol/L and then processed for immunofluorescence or flow cytometry analysis. For immunofluorescence analysis, cells are washed and fixed in 4% paraformaldehyde (PFA) for 15 minutes at room temperature and then permeabilized in PBS+0.1% Triton X-100 for 10 minutes at room temperature. Blocking is performed using 0.5% BSA+0.2% gelatin from cold water fish skin (Sigma, catalog no. G7765) in PBS for 1 hour at room temperature. EdU Click-IT reaction is performed following the manufacturer's instructions. Cells are then washed three times with PBG; primary antibodies are incubated overnight at 4° C., diluted 1:5,000), followed by secondary antibody (anti-mouse, Alexa Fluor 594 (Thermo, A21099, 1:2,000) and DAPI (Thermo Fisher Scientific, D1306, diluted 1 g/mL) for 1 hour at room temperature.

For flow cytometry analysis, media and cells are collected and fixed in PFA 4% for 15 minutes at room temperature followed by EdU Click-IT reaction. After washes, DNA is counterstained with DAPI. Samples where then acquired and analyzed.

### Cell Proliferation Assay:

| | | Cell |
|---|---|---|
| | | density |
| | | (cells/ |
| Cell Line | Culture Medium | well) |
| A2780 | 1640 + 10% FBS + 1% PS + 2 mM Glutamine | 200 |
| SUM149PT | F-12[HAM] + 5% FBS + 1% PS + | 200 |
| | 10 mM HEPES + 1 ug/ml | |
| | Hydrocortisone + 5 ug/ml Insulin | |
| CAL51 | DMEM + 20% FBS + 1% PS | 200 |
| RKO | EMEM + 10% FBS + 1% PS | 200 |
| TOV-112D | 1640 + 10% FBS + 1% PS | 200 |
| HCC1806 | 1640 + 10% FBS + 1% PS | 400 |
| SK-BR-3 | McCoy's 5A + 10% FBS + 1% PS | 800 |
| MDA-MB-436 | DMEM + 10% FBS + 1% PS | 800 |
| DLDI | 1640 + 10% FBS + 1% PS | 300 |
| BRCA2(−/−)- | | |
| mBRCA | | |
| Capan 1 | IMDM + 20% FBS + 1% PS | 4000 |
| UWB1.289 | 50% MEGM + 50% 1640 + 3% FBS + 1% P/S | 600 |
| UWB1.289 + | 50% MEGM + 50% 1640 + 3% FBS + | 200 |
| BRCA1 | 200 ug/ml G418 + 1% P/S | |
| HepG2 | EMEM + 10% FBS + 1% P/S | 1000 |
| A2780-Olaparib | 1640 + 10% FBS + 1% P/S + 20 uM Olaparib | 800 |

### Materials

| Cell or Reagent | Supplier | Cat No. |
|---|---|---|
| Fetal bovine serum (FBS) | HyClone | SH30406.05 |
| PBS | Solarbio | P1020 |
| Trypsin-EDTA solution | Solarbio | Y1300 |
| PS | Solarbio | P1400 |
| DMEM | Gibco | 11965-92 |
| L-15(Leibovitz's) | Gibco | 11415-064 |
| DME/F-12 1:1 | Hyclone | SH30023.01 |
| RPMI1640 medium | Gibco | 11815-093 |
| F-12K | Hyclone | SH30526.01 |
| Cell Counting-Lite 2.0 | Vazyme | DD1101-03 |

Procedure (384 well format):

Cells are counted, diluted to desired density and 40 μL of cell suspension is added into each 384-well tissue-culture treated plates. Plates are incubated in Tissue Culture incubator at 37° C./5% C02 overnight.

Next day, 40 nl test compounds that are prepared from source plates are transferred to the seeded cells from overnight. Plates are incubated at 37° C./5% C02 for a total of 7 days. After 7-day incubation, plates are equilibrated to room temperature and 30 ul of Cell Counting-Lite is added to each well for a 30 min incubation followed by reading on EnVision.

### Results:

Proliferation data is calculated as % control and the results are reported as the IC50 value for each tested compound.

### PAR in Cell Western Assay (Parylation):

| Cell Line | Culture Medium | Cell density(cells/well) |
|---|---|---|
| U2OS | DMEM + 10% FBS + 1% PS | 2667 |

### Materials

| | Vendor | Cat No. | lot No. |
|---|---|---|---|
| DMEM (High Glucose) | GIBCO | 12100 | 240327-09 |
| FBS | Biosera | FB-1058/500 | 010722-UY |
| 1X DPBS | Invitroge | 141900 | 240201-01 |
| 384 well polypropylene plate | Greiner | 781280 | B2207366 |
| LP-0200, ECHO QUALIFIED | LabCYTE | NC9606831 | 6720096 |
| LOW VOLUMEPLAT | | | |
| PDL-Coated CellCarrier-384 ultra/8x20 | PerkinElmer | 6057500 | |
| 0.25% Trypsin-EDTA | Invitrogen Liquid | 25200 | 240307-07 |
| Poly/Mono-ADP Ribose (E6F6A) | CST | 83732S | 5 |
| Rabbit mAb | | | |
| IRDye ® 800CW Goat anti-Rabbit | LI-COR | 926-32211 | D30627-08 |
| IgG Secondary Antibody | | | |
| CellTag ™ 700 Stain for | LI-COR | 926-41090 | D30915-02 |
| In-Cell Western ™ Assays | | | |
| Saruparib | MCE | HY-132167 | 179202 |
| Olaparib | MCE | HY-10162 10mM | 136925 |
| 30% H2O2 | HUSHI | 7722-84-1 | 20230809 |
| IGEPAL CA-630 | sigma | I8896-50ml | MKBJ6286V |
| MeOH | HUSHI | 10014118 | P2935131 |
| TWEEN 20 | sigma | T2700-500ML | MKBJ7237V |
| BSA | ABCONE | B24726-250G | AOB101563 |

### Procedure (384 Well Format):

U2OS cells are seeded at 2667 cells per well in 24 ul medium into poly-D lysine-coated 384 well plate and incubated in the TC incubator for O/N

The next day, test comps are diluted in a source plate and 6 μL of the diluted comp is added to the cell plate; mixed well and incubated for 6 hrs. DNA damage is induced with 2 mM H2O2 for 10 min. After discarding the medium, cells are fixed in ice cold Methanol at for 20 min at −20C. After a wash step at room temperature, cells are permeabilized with 0.05% NP-40 in PBS for 10 min, followed by wash steps. Cells are blocked with 1% BSA, at room temperature for 1 hr. Cells are stained with anti mono/polyADP ribose antibody (E6F6A, Cell signaling, 1:5000) in 1×PBS/1% BSA/0.05% Tween o/n at 4C.

Next Day, cells are washed and incubated with anti-Rabbit 800CV (Licor antibody, 1:500) and CellTag 700 (Licor, 1:800) in 1×PBS/1% BSA/0.05% Tween for 1 h. After washing, plates are scanned using LICOR and are quantified.

### Results:

Parylation inhibition is reported as % control inhibition.

Results are reported as the IC50 value for each tested compound.

### PARP Trapping Assay:

| Cell Line | Culture Medium | Cell density(cells/well) |
|---|---|---|
| U2OS | DMEM + 10% FBS + 1% PS | 15000 |

### Materials

| | Vendor | Cat No. | Lot No. |
|---|---|---|---|
| DMEM (High Glucose) | GIBCO | 12100 | 240327-09 |
| FBS | Biosera | FB-1058/500 | 010722-UY |
| 1X DPBS | Invitroge | 141900 | 240409-01 |
| 384 well polypropylene | Greiner | 781280 | B2207366 |
| plate | | | |
| LP-0200, ECHO | LabCYTE | NC9606831 | 6720096 |
| QUALIFIED | | | |
| LOW VOLUMEPLAT | | | |
| PhenoPlate-96 TC + lid/ | Perkin | 6055302 | 8793-23425 |
| case 2x20B | Elmer | | |
| 0.25% Trypsin-EDTA | Invitrogen | 25200 | 240307-07 |
| | Liquid | | |
| PARP1 antibody | abcam | ab227244 | 1035485-20 |
| aRabbit AF488 | Thermo | A-11008 | 2557379 |
| | Fisher | | |
| DAPI solution | Dojindo | D523 | WR877 |
| Methyl methanesulfonate | selleck | E0609-1g | E060905 |
| Saruparib | MCE | HY-132167 | 179202 |
| Olaparib | MCE | HY-10162 10mM | 136925 |
| Triton X-100 | sigma | X100-100ml | WD317484 |
| Sodium chloride | sigma | s7663-1kg | 7647-14-5 |
| sucrose | sigma | V900116-500g | WXBD9010V |
| Magnesium chloride | sigma | M2670-500G | 7791-18-6 |
| hexahydrate | | | |
| PIPES | sigma | V900404-100G | WXBC9242V |
| NaOH | Hushi | 10019718 | 20200724 |
| MeOH | HUSHI | 10014118 | P2935131 |
| TWEEN 20 | sigma | T2700-500ML | MKBJ7237V |
| BSA | ABCONE | B24726-250G | AOB101563 |

### Procedure (96 Well Format):

U2OS cells are seeded at 15,000 cells/well in 90 ul medium and incubated in the TC incubator for overnight.

Next day, test compounds are prepared in a source plate along with addition of DNA damage reagent MMS simultaneously and 10 ul of the diluted mixture is added to the cell plate and incubated for 4 hrs under T/C conditions.

After 4 hrs, following aspiration and washing, add 100 ul of cold cytoskeleton removal buffer (0.5% Triton X-100) and incubated for 10 min. At the end of incubation, slowly the buffer is removed, and cells are fixed with ice cold MeOH for 15 min at −20C. Followed by wash steps, cells are blocked with 1×PBS/3% BSA/0.05% Tween for 1 h at room temperature.

Cells are stained with anti PARP1 antibody (abeam ab227244, 1:1000), in 1×PBS/1% BSA overnight at 4C.

Next day wash and incubate cells with anti-Rabbit AF488 (Thermo Fisher A-11008, 1:500) in 1×PBS/1% BSA for 1 h at room temp. Wash and add DAPI, scan cells in Celigo.

Data Analysis: DAPI staining is used to segment nuclei and AF488 fluorescence intensity per nucleus is measured.

### Result:

% Trapped Parp is reported in IC50 values for the tested comps (Table 3).

### DeUbiquitination Assay Using Denaturing Buffer and SignalSeeker Ubiquitination Affinity Beads

| Cell Line | Culture Medium | Cell density(cells/10 cm) |
|---|---|---|
| U2OS | McCoys 5A + 10% FBS + 1% PS | 3 × 10\hat{ }6 |

### Materials

| | Vendor | Cat No. | lot No. |
|---|---|---|---|
| McCoy's 5A | GIBCO | 16600082 | |
| FBS | Biosera | FB-1058/500 | 010722-UY |
| 1X DPBS | Invitrogen | 141900 | 240201-01 |
| Penicillin-Streptomycin | Gibco | 15140122 | |
| 0.25% Trypsin-EDTA | Invitrogen | 25200 | 240307-07 |
| | Liquid | | |
| HEPES | | | |
| Triton X-100 | sigma | X100-100ml | WD317484 |
| Sodium chloride | sigma | s7663-1kg | 7647-14-5 |
| EDTA | | | |
| Glycerol | | | |
| PMSF | Sigma | Cat#93482- | |
| | | 50ML-F | |
| HALT protease Inhibitor | Pierce | 87786 | |
| NEM | Sigma | E3876 | |
| PR619 | Lifesensors | S19619 | |
| Signal-Seeker Ubiquitination detection Kit | Cytoskeleton | BK161 | 075 |
| TUBE washing, elution and | LifeSensors | UM311-3000 | UM48503.001 |
| Neutralization buffer | | | |
| 5x Protein Loading Buffer | | | |
| PARP1 antibody | abcam | ab227244 | 1035485-21 |
| Actin Antibody | Sigma | A1978 | 0000128052 |
| Rabbit IRDye 800CW | Licor | 926-32211 | D030627-05 |
| Mouse IR Dye 680RD | Licor | 926-68070 | D30207-05 |
| Revert 700 Total Protein Stain Kit | Licor | 92611010 | D30802-01 |
| NuPAGE Novex 4-12 Bis-Tris Midi Gels | Thermo Fisher | WG1403BOX | |
| MOPS Buffer | Invitrogen | NP000102 | |
| Nitrocellulose Membrane | Amersham | 10600002 | |

### Procedure (10 cm):

U2OS cells are seeded at 3×10\hat{ }6 cells per 10 cm dish and incubated in the TC incubator for O/N.

The next day, 1000× test comps are diluted at 5 uL in 5 ml of medium for the appropriate concentration, mixed well by vortexing and added to the cells. Cells are incubated for 2 hours. The DNA alkylating agent MMS is then added to a final concentration of 0.01% induce DNA damage and PARP ubiquitination. Cells are incubated for an additional 4 hours.

Cells are washed with DPBS, trypsinized, resuspended in media and spun down for collection. The cell pellet is resuspended in 1 ml of cold PBS, transferred to a 1.5 ml tube, and spun down for cell collection. PBS is aspirated, and the cell pellet is flash frozen in LN2 and stored at −80C.

For the cell lysis, the cell pellet is lysed using 150 uL 1% SDS lysis buffer. The crude lysate is transferred to a Blast® filter placed in a 1.5 mL tube and compressed through. After centrifugation at 10000×g for 1 min, 900 uL Lysis buffer+inhibitors are added to dilute SDS from the samples. The total protein is quantified using BCA analysis.

For the ubiquitin pulldown, 30 ul bead slurry of CS Ubiquitination affinity beads per sample are equilibrated, 500 μg of lysate are added and beads are incubated by rotation over end for 2 hours at 4C. After incubation, the beads are washed 1× with CS BlastR Wash buffer, 3× with 1×DPBS-Tween (PBS+0.1% Tween-20), then with 50 μL of Lifesensors Wash buffer. In between each wash steps the beads are collected by centrifugation for 4000 g for 1 min. Beads are then eluted in 18 μL of LifeSensors elution buffer for 15 min at RT. The eluate is collected, and 2 ul of neutralization buffer and 5 ul of 5× loading dye are added. To control for protein concentration and loading, 10 μg of lysate are mixed with loading dye as an input sample. Pulldowns and input samples are boiled for 5 min at 95C and are undergoing Western blotting according to standard methods using the indicated antibodies.

### Results:

PARP ubiquitination is measured by quantifying the PARP1 Ub smear and normalizing it to the PARP1 levels in the input samples. Results are reported as yes or no for deubiquitination by comparison of the ubiquitination signal of test compound treated samples ton Olaparib.

### DeUbiquitination Assay Using Nuclear Extract and Lifesensors Ubiquitination TUBES Affinity Beads

| Cell Line | Culture Medium | Cell density(cells/10 cm) |
|---|---|---|
| U2OS | McCoys 5A + 10% FBS + 1% PS | 3 × 10\hat{ }6 |

### Materials

| | Vendor | Cat No. | lot No. |
|---|---|---|---|
| McCoy's 5A | GIBCO | 16600082 | |
| FBS | Biosera | FB-1058/500 | 010722-UY |
| 1X DPBS | Invitrogen | 141900 | 240201-01 |
| Penicillin-Streptomycin | Gibco | 15140122 | |
| 0.25% Trypsin-EDTA | Invitrogen Liquid | 25200 | 240307-07 |
| NE-PER Nuclear and Cytoplasmic | Thermo Fisher | 78835 | |
| Extraction reagents | | | |
| EDTA | | | |
| PMSF | Sigma | Cat#93482- | |
| | | 50ML-F | |
| HALT protease Inhibitor | Pierce | 87786 | |
| NEM | Sigma | E3876 | |
| PR619 | Lifesensors | S19619 | |
| TUBE 1 beads | LifeSensors | UM-0501M- | UM-48488- |
| | | 1000 | 001 |
| TUBE washing, elution and | LifeSensors | UM311-3000 | UM48503.001 |
| Neutralization buffer | | | |
| 5x Protein Loading Buffer | | | |
| PARP1 antibody | abcam | ab227244 | 1035485-21 |
| Actin Antibody | Sigma | A1978 | 0000128052 |
| Lamin A/C Mouse mAb | | | |
| Rabbit IRDye 800CW | Licor | 926-32211 | D030627-05 |
| Mouse IR Dye 680RD | Licor | 926-68070 | D30207-05 |
| Revert 700 Total Protein Stain Kit | Licor | 92611010 | D30802-01 |
| NuPAGE Novex 4-12 Bis-Tris Midi Gels | Thermo Fisher | WG1403BOX | |
| MOPS Buffer | Invitrogen | NP000102 | |
| Nitrocellulose Membrane | Amersham | 10600002 | |

### Procedure (10 cm):

U2OS cells are seeded at 3×10\hat{ }6 cells per 10 cm dish and incubated in the TC incubator for O/N.

The next day, 1000× test comps are diluted at 5 uL in 5 ml of medium for the appropriate concentration, mixed well by vortexing and added to the cells. Cells are incubated for 2 hours. The DNA alkylating agent MMS is added to a final concentration of 0.01% induce DNA damage, which will increase PARP ubiquitination. Cells are incubated for an additional 4 hours.

Cells are washed with DPBS, trypsinized, resuspended in media and spun down for collection. The cell pellet is resuspended in 1 ml of cold PBS, transferred to a 1.5 ml tube, and the sample is spun down. PBS is aspirated, and cells are processed for nuclear extraction.

For the nuclear extraction, the cell pellet is resuspended in 400 ul ice-cold CER I (NE-PER, plus inhibitors), vortexed vigorously and incubated on ice for 10 minutes before 22 ul ice-cold CER II is added to the tube. The sample is mixed again and incubated for an additional minute. After vortexing, the tube is centrifuged for 5 min at maximum speed. The supernatant (cytoplasmic extract) is transferred to a new tube and the pellet, which contains nuclei, is resuspended in 200 ul ice-cold NER (NE-PER, plus inhibitors). The sample is incubated for 40 min on ice with continuous vortexing, then flash frozen and stored at −80C. The next day the nuclear extract sample centrifuged at maximum speed for 10 min and the supernatant (nuclear extract) collected in a new tube. The total protein is quantified using BCA analysis.

For the ubiquitin pulldown, 50 ul bead slurry of TUBE1 Ubiquitination affinity beads per sample are equilibrated, 100 μg of nuclear extract are added and beads are incubated by rotation over end for 2 hours at 4C. After incubation, the beads are washed 3× with 1×DPBS-Tween (PBS+0.1% Tween-20), and then 50 μL of Lifesensors Wash buffer for 5 min. In between each wash steps the magnetic beads are collected on a magnetic stand. Beads are then eluted in 18 μL of LifeSensors elution buffer for 15 min at RT. The eluate is collected, and 2 ul of neutralization buffer and 5 ul of 5× loading dye are added. To control for protein concentration and loading, 10 ug of lysate are mixed with loading dye as an input sample. Pulldowns and input samples are boiled for 5 min at 95C and are undergoing Western blotting according to standard methods using the indicated antibodies.

### Results:

PARP ubiquitination is measured by quantifying the PARP1 Ub smear and normalizing it to the PARP1 levels in the input samples. Results are reported as yes or no for deubiquitination by comparison of the ubiquitination signal of test compound treated samples to Olaparib.

### Biochemical Assay:

The PARP enzyme catalyzes the transfer of ADP-Ribose from NAD+ onto PAR chains/histone substrates. The catalytic cycle is monitored indirectly with the NAD/NADH-Glo Assay kit from Promega. Compounds are typically tested in a 10-concentration response curve with a 3-fold serial dilution starting at 1.0 or 0.1 μM. Veliparib (ABT-888) is the internal control compound tested starting at 0.1 μM. NAD+ consumed is determined by comparison to the no enzyme control.

### Reagents:

Recombinant PARP1
NAD+/NAHD-Glow assay Kit
Activated DNA, 10 μg/mL
NAD+(Nicotinamide Adenine Dinucleotide), 500 nM
Protein substrate for PARP1: Core Histone from chicken, 10 μg/mL

Table 3 shows the results of the PARP1-enzyme assay, PARP Trapping in U2OS cells, and parylation assay in U2OS cells. The letter codes for the PARP1-enzyme assay include: A (<1 nM), B (>1-10 nM), C (>10-100 nM), D (>100-1000 nM), E (>1000 nM), and “-” is not tested. The letter codes for the PARP trapping assay, and parylation assay include: A (<10 nM), B (>10-100 nM), C (>100-1000 nM), D (>1000-1000 nM), E (>10000 nM), and “-” is not tested.

TABLE 3

| I-#/ | PARP1-Enzyme | PARP Trapping | Parylation |
|---|---|---|---|
| Compound | Assay | (U2OS) | (U2OS) |
| Olaparib | A | D | A |
| I-61 | A | B | C |
| I-62 | A | B | C |
| I-63 | A | C | C |
| I-64 | A | C | C |
| I-28 | A | C | C |
| I-29 | A | — | A |
| I-30 | A | C | C |
| I-31 | A | — | A |
| I-173 | A | B | C |
| I-167 | A | B | C |
| I-169 | A | B | C |
| I-168 | A | B | C |
| I-183 | A | C | C |
| I-184 | A | C | C |
| I-166 | A | B | C |
| I-157 | A | C | C |
| I-165 | A | C | C |
| I-193 | A | E | C |
| I-194 | A | E | C |
| I-195 | A | D | B |
| I-238 | A | C | C |
| I-233 | A | C | B |
| I-366 | A | C | C |
| I-333 | — | C | B |
| I-308 | — | B | B |
| I-294 | B | C | B |
| I-295 | A | C | B |
| I-331 | A | C | B |
| I-353 | A | C | C |
| I-962 | A | C | B |
| I-365 | A | C | B |
| I-369 | A | C | B |
| I-367 | A | C | C |
| I-963 | A | | C |
| I-323 | A | C | C |
| I-346 | A | C | B |
| I-354 | A | B | C |
| I-373 | A | C | C |
| I-374 | A | C | C |
| I-787 | A | B | B |
| I-404 | A | C | B |
| I-418 | A | C | C |
| I-377 | A | B | B |
| I-379 | B | B | B |
| I-407 | A | B | B |
| I-410 | A | B | B |
| I-420 | A | B | C |
| I-402 | — | C | B |
| I-412 | — | B | C |
| I-378 | — | B | B |
| I-380 | — | B | B |
| I-382 | — | B | B |
| I-403 | A | C | B |
| I-409 | — | C | C |
| I-419 | — | C | B |
| I-453 | — | C | C |
| I-454 | — | C | B |
| I-381 | — | A | C |
| I-457 | A | B | C |
| I-800 | — | B | C |
| I-469 | — | B | B |
| I-470 | — | B | C |
| I-803 | — | C | B |
| I-489 | — | C | B |
| I-818 | — | C | B |
| I-528 | — | C | B |
| I-590 | — | B | A |
| I-634 | — | B | A |
| I-635 | — | B | A |
| I-637 | — | B | C |
| I-660 | — | B | C |
| I-646 | — | B | B |
| I-690 | — | B | B |
| I-661 | — | B | C |
| I-709 | — | B | A |
| I-727 | — | A | A |
| I-715 | — | B | B |
| I-871 | — | B | B |
| I-743 | — | B | B |
| I-745 | — | B | B |
| I-870 | — | B | B |
| I-875 | — | C | C |
| I-750 | — | B | B |
| I-739 | — | B | A |
| I-749 | — | B | B |
| I-892 | — | B | C |
| I-893 | — | B | C |
| I-771 | — | B | A |
| I-772 | — | A | B |
| I-883 | — | B | B |
| I-768 | — | B | B |
| I-769 | — | A | A |
| I-884 | — | B | C |
| I-910 | — | — | C |
| I-774 | — | — | B |
| I-777 | — | — | B |
| I-971 | — | B | B |
| I-973 | — | — | C |
| I-993 | — | B | B |
| I-994 | — | — | C |
| I-990 | — | B | B |

Table 4 shows the results of the cell proliferation assay in A2780, HCC1806, and MDA-MB-436 cells. The letter codes for the cell proliferation assay in A2780, HCC1806, and MDA-MB-436 cells include: A (<10 nM), B (>10-100 nM), C (>100-1000 nM), D (>1000-10000 nM), E (>10000 nM), and “-” is not tested.

TABLE 4

| | Cell | Cell | Cell |
|---|---|---|---|
| I-#/ | proliferation | Proliferation | Proliferation |
| Compound | (A2780) | (HCC1806) | (MDA-MB-436) |
| Olaparib | D | D | B |
| I-61 | C | — | B |
| I-62 | C | — | B |
| I-63 | B | C | C |
| I-64 | C | — | B |
| I-28 | D | C | B |
| I-29 | — | A | D |
| I-30 | D | — | B |
| I-31 | — | A | C |
| I-173 | — | A | B |
| I-167 | — | A | C |
| I-169 | C | — | B |
| I-168 | B | — | A |
| I-183 | C | — | B |
| I-184 | C | — | B |
| I-166 | — | A | B |
| I-157 | — | A | B |
| I-165 | — | A | B |
| I-193 | — | A | C |
| I-194 | — | A | C |
| I-195 | — | A | C |
| I-238 | D | — | B |
| I-233 | — | A | B |
| I-366 | C | C | C |
| I-333 | C | — | B |
| I-308 | C | — | C |
| I-294 | C | — | B |
| I-295 | C | — | B |
| I-331 | B | — | B |
| I-353 | C | — | B |
| I-962 | C | C | B |
| I-365 | D | — | B |
| I-369 | B | C | A |
| I-367 | C | C | C |
| I-963 | C | — | C |
| I-323 | C | — | A |
| I-346 | C | — | B |
| I-354 | B | — | B |
| I-373 | C | — | C |
| I-374 | B | — | B |
| I-787 | B | B | A |
| I-404 | B | — | A |
| I-418 | C | — | A |
| I-377 | B | — | A |
| I-379 | B | — | A |
| I-407 | B | — | A |
| I-410 | B | B | A |
| I-420 | C | — | A |
| I-402 | B | B | A |
| I-412 | B | — | A |
| I-378 | B | — | A |
| I-380 | B | — | A |
| I-382 | B | — | A |
| I-403 | B | B | A |
| I-409 | C | — | A |
| I-419 | B | — | A |
| I-453 | B | — | B |
| I-454 | B | — | C |
| I-381 | B | — | B |
| I-457 | B | B | A |
| I-800 | C | C | A |
| I-469 | B | B | A |
| I-470 | B | B | A |
| I-803 | B | C | A |
| I-489 | B | C | A |
| I-818 | B | C | A |
| I-528 | B | C | A |
| I-590 | A | B | A |
| I-634 | — | B | A |
| I-635 | A | B | A |
| I-637 | — | B | C |
| I-660 | — | B | A |
| I-646 | C | B | A |
| I-690 | C | B | A |
| I-661 | — | B | A |
| I-709 | — | B | A |
| I-727 | — | B | A |
| I-715 | — | B | A |
| I-871 | C | C | A |
| I-743 | — | B | A |
| I-745 | — | B | A |
| I-870 | B | B | A |
| I-875 | C | C | A |
| I-750 | B | B | A |
| I-739 | C | C | A |
| I-749 | B | B | A |
| I-892 | C | C | B |
| I-893 | C | C | B |
| I-771 | C | C | A |
| I-772 | C | C | A |
| I-883 | B | B | A |
| I-768 | C | C | A |
| I-769 | B | B | A |
| I-884 | B | B | A |
| I-910 | B | B | A |
| I-774 | B | B | A |
| I-777 | B | B | A |
| I-971 | B | C | A |
| I-973 | C | C | B |
| I-993 | B | B | A |
| I-994 | B | B | A |
| I-990 | B | B | A |

LENGTHY TABLES
The patent application contains a lengthy table section. A copy of the table is available in electronic form from the USPTO web site (https://seqdata.uspto.gov/?pageRequest=docDetail&DocID=US20250127907A1). An electronic copy of the table will also be available from the USPTO upon request and payment of the fee set forth in 37 CFR 1.19(b)(3).

1. A compound of formula I, 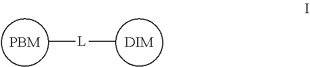
or a pharmaceutically acceptable salt thereof, wherein:
PBM is a protein binding moiety capable of binding PARP;
L is a bivalent moiety that connects PBM to DIM; and
DIM is a deubiquitination inducing moiety.

2. The compound of claim 1, wherein PBM is a PARP binding moiety thereby forming a compound of formula I-a: 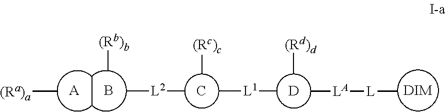
or a pharmaceutically acceptable salt thereof, wherein;
Ring A and Ring B are independently fused rings selected from benzo, a 4-7 membered saturated or partially unsaturated carbocyclyl, a 4-7 membered saturated or partially unsaturated heterocyclyl with 1-3 heteroatoms independently selected from nitrogen, oxygen, and sulfur, and a 5-6 membered heteroaryl with 1-4 heteroatoms independently selected from nitrogen, oxygen and sulfur;
Ring C is a ring selected from phenylenyl, a 4-7 membered saturated or partially unsaturated carbocyclylenyl, a 4-7 membered saturated or partially unsaturated heterocyclylenyl with 1-3 heteroatoms independently selected from nitrogen, oxygen, and sulfur, and a 5-6 membered heteroarylenyl with 1-4 heteroatoms independently selected from nitrogen, oxygen and sulfur;
Ring D is a ring selected from phenylenyl, a 4-7 membered saturated or partially unsaturated carbocyclylenyl, a 4-7 membered saturated or partially unsaturated heterocyclylenyl with 1-3 heteroatoms independently selected from nitrogen, oxygen, and sulfur, and a 5-6 membered heteroarylenyl with 1-4 heteroatoms independently selected from nitrogen, oxygen and sulfur;
L¹, L² and L^{A} are independently a covalent bond or a bivalent, saturated or partially unsaturated, straight or branched C₁₋₅ hydrocarbon chain, wherein 0-3 methylene units of L¹, L² or L^{A} are independently replaced by —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, or —OC(O)NR—, —NRC(O)O—.
each R^{a}, R^{b}, R^{c}, and R^{d} is independently selected from hydrogen, R^{A}, halogen, —CN, —NO₂, —OR, —SR, —NR₂, —S(O)₂R, —S(O)₂NR₂, —S(O)R, —C(O)R, —C(O)OR, —C(O)NR₂, —C(O)NROR, —OC(O)R, —OC(O)NR₂, —NRC(O)OR, —NRC(O)R, —NRC(O)N(R)₂, and —NRS(O)₂R;
each R^{A} is independently an optionally substituted group selected from C₁₋₆ aliphatic, phenyl, a 3-12 membered saturated or partially unsaturated monocyclic, bicyclic, bridged bicyclic or spirocyclic carbocyclyl or heterocyclyl with 1-3 heteroatoms independently selected from nitrogen, oxygen, and sulfur, and a 5-6 membered heteroaryl ring having 1-4 heteroatoms independently selected from nitrogen, oxygen, and sulfur;
each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
two R groups on the same nitrogen are optionally taken together with their intervening atoms to form a 4-7 membered saturated, partially unsaturated, or heteroaryl ring having 0-3 heteroatoms, in addition to the nitrogen atom to which they are attached, independently selected from nitrogen, oxygen, and sulfur;
a, b, c, and d are independently 0, 1, 2, 3, or 4;
L is a covalent bond or a bivalent, saturated or partially unsaturated, straight or branched C₁₋₅₀ hydrocarbon chain, wherein 0-6 methylene units of L are independently replaced by -Cy-, —O—, —NR—, —SiR₂—, —Si(OH)R—, —Si(OH)₂—, —P(O)OR—, —P(O)R—, —P(O)NR₂—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, —NRC(O)O—, 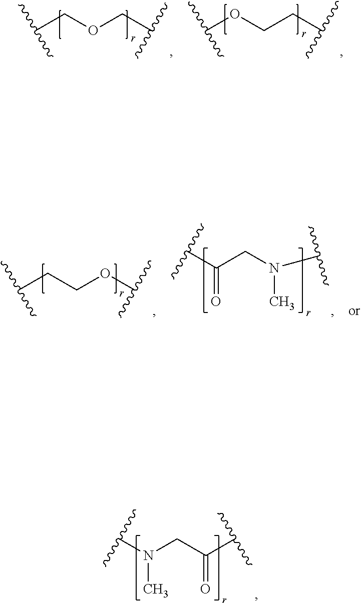
wherein;
each -Cy- is independently an optionally substituted bivalent ring selected from phenylenyl, an 8-10 membered bicyclic arylenyl, a 4-7 membered saturated or partially unsaturated carbocyclylenyl, a 6-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-2 heteroatoms independently selected from nitrogen, oxygen, and sulfur, a 6-11 membered saturated or partially unsaturated spiro heterocyclylenyl having 1-2 heteroatoms independently selected from nitrogen, oxygen, and sulfur, an 8-10 membered bicyclic saturated or partially unsaturated heterocyclylenyl having 1-2 heteroatoms independently selected from nitrogen, oxygen, and sulfur, a 5-6 membered heteroarylenyl having 1-4 heteroatoms independently selected from nitrogen, oxygen, and sulfur, and an 8-10 membered bicyclic heteroarylenyl having 1-5 heteroatoms independently selected from nitrogen, oxygen, and sulfur; and
r is 0, 1, 2, 3, 4, 5, 6, 7, 8, 9, or 10;
or
wherein PBM is a PARP binding moiety thereby forming a compound of formula I-b: 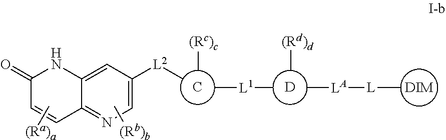
or a pharmaceutically acceptable salt thereof,
wherein;
Ring C is a ring selected from phenylenyl, a 4-7 membered saturated or partially unsaturated carbocyclylenyl, a 4-7 membered saturated or partially unsaturated heterocyclenyl with 1-3 heteroatoms independently selected from nitrogen, oxygen, and sulfur, and a 5-6 membered heteroarylenyl with 1-4 heteroatoms independently selected from nitrogen, oxygen and sulfur;
Ring D is a ring selected from phenylenyl, a 4-7 membered saturated or partially unsaturated carbocyclylenyl, a 4-7 membered saturated or partially unsaturated heterocyclylenyl with 1-3 heteroatoms independently selected from nitrogen, oxygen, and sulfur, and a 5-6 membered heteroarylenyl with 1-4 heteroatoms independently selected from nitrogen, oxygen and sulfur;
L¹, L², and L^{A} are independently a covalent bond or a bivalent, saturated or partially unsaturated, straight or branched C₁₋₂ hydrocarbon chain, wherein 0-3 methylene units of L¹, L² or L^{A} are independently replaced by —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, or —OC(O)NR—, or —NRC(O)O—;
each R^{a}, R^{b}, R^{c}, and R^{d} is independently selected from hydrogen, R^{A}, halogen, —CN, —NO₂, —OR, —SR, —NR₂, —S(O)₂R, —S(O)₂NR₂, —S(O)R, —C(O)R, —C(O)OR, —C(O)NR₂, —C(O)NROR, —OC(O)R, —OC(O)NR₂, —NRC(O)OR, —NRC(O)R, —NRC(O)N(R)₂, and —NRS(O)₂R;
each R^{A} is independently an optionally substituted group selected from C₁₋₆ aliphatic, phenyl, a 3-12 membered saturated or partially unsaturated monocyclic, bicyclic, bridged bicyclic or spirocyclic carbocyclyl or heterocyclyl with 1-3 heteroatoms independently selected from nitrogen, oxygen, and sulfur, and a 5-6 membered heteroaryl ring having 1-4 heteroatoms independently selected from nitrogen, oxygen, and sulfur;
each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
two R groups on the same nitrogen are optionally taken together with their intervening atoms to form a 4-7 membered saturated, partially unsaturated, or heteroaryl ring having 0-3 heteroatoms, in addition to the nitrogen atom to which they are attached, independently selected from nitrogen, oxygen, and sulfur:
a, b, c, and d are independently 0, 1, 2, 3, or 4;
L is a covalent bond or a bivalent, saturated or partially unsaturated, straight or branched C₁₋₅₀ hydrocarbon chain, wherein 0-6 methylene units of L are independently replaced by -Cy-, —O—, —NR—, —SiR₂—, —Si(OH)R—, —Si(OH)₂—, —P(O)OR—, —P(O)R—, —P(O)NR₂—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, —NRC(O)O—, 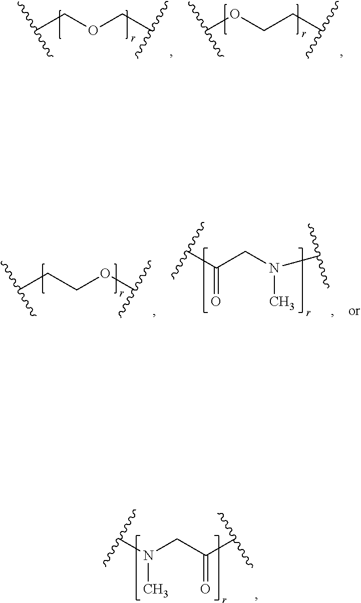
r wherein;
each -Cy- is independently an optionally substituted bivalent ring selected from phenylenyl, an 8-10 membered bicyclic arylenyl, a 4-7 membered saturated or partially unsaturated carbocyclylenyl, a 6-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-2 heteroatoms independently selected from nitrogen, oxygen, and sulfur, a 6-11 membered saturated or partially unsaturated spiro heterocyclylenyl having 1-2 heteroatoms independently selected from nitrogen, oxygen, and sulfur, an 8-10 membered bicyclic saturated or partially unsaturated heterocyclylenyl having 1-2 heteroatoms independently selected from nitrogen, oxygen, and sulfur, a 5-6 membered heteroarylenyl having 1-4 heteroatoms independently selected from nitrogen, oxygen, and sulfur, and an 8-10 membered bicyclic heteroarylenyl having 1-5 heteroatoms independently selected from nitrogen, oxygen, and sulfur; and
r is 0, 1, 2, 3, 4, 5, 6, 7, 8, 9, or 10; or
wherein PBM is a PARP binding moiety thereby forming a compound of formula I-c: 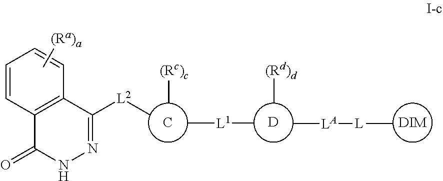
or a pharmaceutically acceptable salt thereof,
wherein:
Ring C is a ring selected from phenylenyl, a 4-7 membered saturated or partially unsaturated carbocyclylenyl, a 4-7 membered saturated or partially unsaturated heterocyclylenyl with 1-3 heteroatoms independently selected from nitrogen, oxygen, and sulfur, and a 5-6 membered heteroarylenyl with 1-4 heteroatoms independently selected from nitrogen, oxygen and sulfur;
Ring D is a ring selected from phenylenyl, a 4-7 membered saturated or partially unsaturated carbocyclylenyl, a 4-7 membered saturated or partially unsaturated heterocyclylenyl with 1-3 heteroatoms independently selected from nitrogen, oxygen, and sulfur, and a 5-6 membered heteroarylenyl with 1-4 heteroatoms independently selected from nitrogen, oxygen and sulfur;
L¹, L² and L^{A} are independently a covalent bond or a bivalent, saturated or partially unsaturated, straight or branched C₁₋₅ hydrocarbon chain, wherein 0-3 methylene units of L¹, L² or L^{A} are independently replaced by —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, or —OC(O)NR—, —NRC(O)O—.
each R^{a}, R^{b}, R^{c}, and R^{d} is independently selected from hydrogen, R^{A}, halogen, —CN, —NO₂, —OR, —SR, —NR₂, —S(O)₂R, —S(O)₂NR₂, —S(O)R, —C(O)R, —C(O)OR, —C(O)NR₂, —C(O)NROR, —OC(O)R, —OC(O)NR₂, —NRC(O)OR, —NRC(O)R, —NRC(O)N(R)₂, and —NRS(O)₂R;
each R^{A} is independently an optionally substituted group selected from C₁₋₆ aliphatic, phenyl, a 3-12 membered saturated or partially unsaturated monocyclic, bicyclic, bridged bicyclic or spirocyclic carbocyclyl or heterocyclyl with 1-3 heteroatoms independently selected from nitrogen, oxygen, and sulfur, and a 5-6 membered heteroaryl ring having 1-4 heteroatoms independently selected from nitrogen, oxygen, and sulfur;
each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
two R groups on the same nitrogen are optionally taken together with their intervening atoms to form a 4-7 membered saturated, partially unsaturated, or heteroaryl ring having 0-3 heteroatoms, in addition to the nitrogen atom to which they are attached, independently selected from nitrogen, oxygen, and sulfur:
a, b, c, and d are independently 0, 1, 2, 3, or 4;
L is a covalent bond or a bivalent, saturated or partially unsaturated, straight or branched C₁₋₅₀ hydrocarbon chain, wherein 0-6 methylene units of L are independently replaced by -Cy-, —O—, —NR—, —SiR₂—, —Si(OH)R—, —Si(OH)₂—, —P(O)OR—, —P(O)R—, —P(O)NR₂—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, —NRC(O)O—, 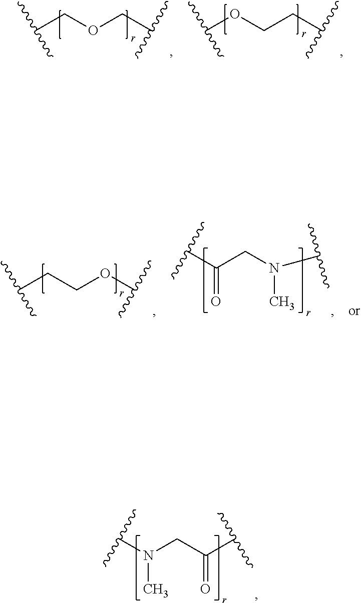
wherein;
each -Cy- is independently an optionally substituted bivalent ring selected from phenylenyl, an 8-10 membered bicyclic arylenyl, a 4-7 membered saturated or partially unsaturated carbocyclylenyl, a 6-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-2 heteroatoms independently selected from nitrogen, oxygen, and sulfur, a 6-11 membered saturated or partially unsaturated spiro heterocyclylenyl having 1-2 heteroatoms independently selected from nitrogen, oxygen, and sulfur, an 8-10 membered bicyclic saturated or partially unsaturated heterocyclylenyl having 1-2 heteroatoms independently selected from nitrogen, oxygen, and sulfur, a 5-6 membered heteroarylenyl having 1-4 heteroatoms independently selected from nitrogen, oxygen, and sulfur, and an 8-10 membered bicyclic heteroarylenyl having 1-5 heteroatoms independently selected from nitrogen, oxygen, and sulfur; and
r is 0, 1, 2, 3, 4, 5, 6, 7, 8, 9, or 10.

3-4. (canceled)

5. The compound of claim 2, wherein Ring C is phenylene.

6-7. (canceled)

8. The compound of claim 2, wherein Ring D is 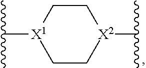
wherein X¹ and X² are independently carbon or nitrogen.

9-12. (canceled)

13. The compound of claim 2, wherein the compound is of one of the following formulae: 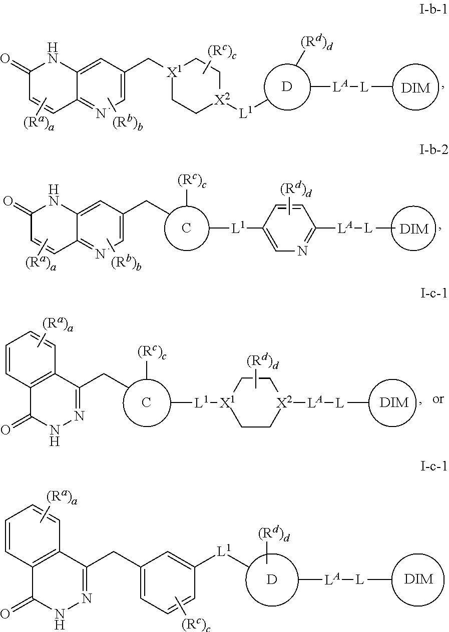
or a pharmaceutically acceptable salt thereof, wherein X¹ and X² are independently carbon or nitrogen.

14-15. (canceled)

16. The compound of claim 1, wherein DIM is a deubiquitinase binding moiety (DBM) and said compound is of formula I-bb: 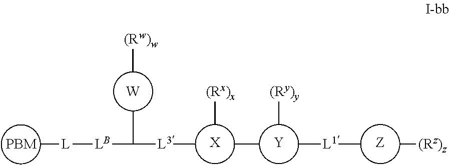
or a pharmaceutically acceptable salt thereof, wherein;
Ring Z is a ring selected from phenyl, a 4-7 membered saturated or partially unsaturated carbocyclic ring, a 4-7 membered saturated or partially unsaturated heterocyclic ring with 1-3 heteroatoms independently selected from nitrogen, oxygen, and sulfur, and a 5-6 membered monocyclic heteroaromatic ring with 1-4 heteroatoms independently selected from nitrogen, oxygen and sulfur;
Ring Y is a ring selected from phenylenyl, a 4-7 membered saturated or partially unsaturated carbocyclylenyl, a 4-7 membered saturated or partially unsaturated heterocyclylenyl with 1-3 heteroatoms independently selected from nitrogen, oxygen, and sulfur, and a 5-6 membered heteroarylenyl with 1-4 heteroatoms independently selected from nitrogen, oxygen and sulfur;
Ring X is a ring selected from phenylenyl, a 4-7 membered saturated or partially unsaturated carbocyclylenyl, a 4-7 membered saturated or partially unsaturated heterocyclylenyl with 1-3 heteroatoms independently selected from nitrogen, oxygen, and sulfur, and a 5-6 membered heteroarylenyl with 1-4 heteroatoms independently selected from nitrogen, oxygen and sulfur;
Ring W is a ring selected from phenylenyl, a 4-7 membered saturated or partially unsaturated carbocyclylenyl, a 4-7 membered saturated or partially unsaturated heterocyclylenyl with 1-3 heteroatoms independently selected from nitrogen, oxygen, and sulfur, and a 5-6 membered heteroarylenyl with 1-4 heteroatoms independently selected from nitrogen, oxygen and sulfur;
each L^{B}, L^{1′}, and L^{3′} is independently a covalent bond or a bivalent, saturated or partially unsaturated, straight or branched C₁₋₅ hydrocarbon chain, wherein 0-3 methylene units of L^{B}, L^{1′}, or L^{3′} are independently replaced by —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, or —OC(O)NR—, —NRC(O)O—;
each R^{w}, R^{x}, R^{y}, and R^{z} is independently selected from hydrogen, R^{B}, halogen, —CN, —NO₂, —OR, —SR, —NR₂, —S(O)₂R, —S(O)₂NR₂, —S(O)R, —C(O)R, —C(O)OR, —C(O)NR₂, —C(O)NROR, —OC(O)R, —OC(O)NR₂, —NRC(O)OR, —NRC(O)R, —NRC(O)N(R)₂, and —NRS(O)₂R;
each R^{B} is independently an optionally substituted group selected from C₁₋₆ aliphatic, phenyl, a 3-12 membered saturated or partially unsaturated monocyclic, bicyclic, bridged bicyclic or spirocyclic carbocyclyl or heterocyclyl with 1-3 heteroatoms independently selected from nitrogen, oxygen, and sulfur, and a 5-6 membered heteroaryl ring having 1-4 heteroatoms independently selected from nitrogen, oxygen, and sulfur;
each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
two R groups on the same nitrogen are optionally taken together with their intervening atoms to form a 4-7 membered saturated, partially unsaturated, or heteroaryl ring having 0-3 heteroatoms, in addition to the nitrogen atom to which they are attached, independently selected from nitrogen, oxygen, and sulfur; and
w, x, y, and z are independently 0, 1, 2, 3, or 4; or
said compound is a compound of formula I-bb′: 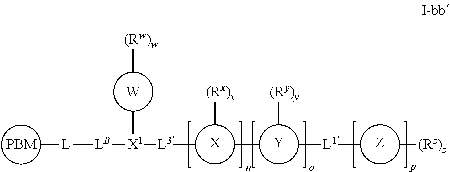
or a pharmaceutically acceptable salt thereof,
wherein;
Ring Z is a ring selected from phenyl, a 3-7 membered saturated or partially unsaturated monocyclic, bicyclic, bridged bicyclic or spirocyclic carbocyclic ring, a 3-7 membered saturated or partially unsaturated monocyclic, bicyclic, bridged bicyclic or spirocyclic heterocyclic ring with 1-3 heteroatoms independently selected from nitrogen, oxygen, and sulfur, and a 5-6 membered heteroaromatic ring with 1-4 heteroatoms independently selected from nitrogen, oxygen and sulfur;
Ring Y is a ring selected from phenylenyl, a 4-8 membered saturated or partially unsaturated carbocyclylenyl, a 4-8 membered saturated or partially unsaturated monocyclic, bicyclic, bridged bicyclic or spirocyclic heterocyclylenyl with 1-3 heteroatoms independently selected from nitrogen, oxygen, and sulfur, and a 5-6 membered heteroarylenyl with 1-4 heteroatoms independently selected from nitrogen, oxygen and sulfur;
Ring X is a ring selected from phenylenyl, a 4-7 membered saturated or partially unsaturated carbocyclylenyl, a 4-7 membered saturated or partially unsaturated heterocyclylenyl with 1-3 heteroatoms independently selected from nitrogen, oxygen, and sulfur, and a 5-6 membered heteroarylenyl with 1-4 heteroatoms independently selected from nitrogen, oxygen and sulfur;
Ring W is a ring selected from phenylenyl, a 4-7 membered saturated or partially unsaturated monocyclic, bicyclic, bridged bicyclic or spirocyclic carbocyclylenyl, a 4-7 membered saturated or partially unsaturated monocyclic, bicyclic, bridged bicyclic or spirocyclic heterocyclylenyl with 1-3 heteroatoms independently selected from nitrogen, oxygen, and sulfur, and a 5-6 membered heteroarylenyl with 1-4 heteroatoms independently selected from nitrogen, oxygen and sulfur;
each L^{B}, L^{1′}, and L^{3′} is independently a covalent bond or an optionally substituted bivalent, saturated or partially unsaturated, straight or branched C₁₋₅ hydrocarbon chain, wherein 0-3 methylene units of L^{B}, L^{1′}, or L^{3′} are independently replaced by —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, or —OC(O)NR—, —NRC(O)O—;
each R^{w}, R^{x}, R^{y}, and R^{z} is independently selected from hydrogen, R^{B}, halogen, —CN, —NO₂, —OR, —SR, —NR₂, —S(O)₂R, —S(O)₂NR₂, —S(O)R, —C(O)R, —C(O)OR, —C(O)NR₂, —C(O)NROR, —OC(O)R, —OC(O)NR₂, —NRC(O)OR, —NRC(O)R, —NRC(O)N(R)₂, and —NRS(O)₂R;
each R^{B} is independently an optionally substituted group selected from C₁₋₆ aliphatic, phenyl, a 3-12 membered saturated or partially unsaturated monocyclic, bicyclic, bridged bicyclic or spirocyclic carbocyclyl or heterocyclyl with 1-3 heteroatoms independently selected from nitrogen, oxygen, and sulfur, and a 5-6 membered heteroaryl ring having 1-4 heteroatoms independently selected from nitrogen, oxygen, and sulfur;
each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
two R groups on the same nitrogen are optionally taken together with their intervening atoms to form a 4-7 membered saturated, partially unsaturated, or heteroaryl ring having 0-3 heteroatoms, in addition to the nitrogen atom to which they are attached, independently selected from nitrogen, oxygen, and sulfur;
X¹ is carbon or nitrogen;
m, n, o, and p are independently 0 or 1; and
w, x, y, and z are independently 0, 1, 2, 3, or 4; or
said compound is a compound of formula I-cc: 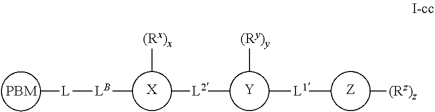
or a pharmaceutically acceptable salt thereof, wherein;
Ring Z is a ring selected from phenyl, a 4-7 membered saturated or partially unsaturated carbocyclic ring, a 4-7 membered saturated or partially unsaturated heterocyclic ring with 1-3 heteroatoms independently selected from nitrogen, oxygen, and sulfur, and a 5-6 membered heteroaromatic ring with 1-4 heteroatoms independently selected from nitrogen, oxygen and sulfur;
Ring Y is a ring selected from phenylenyl, a 4-7 membered saturated or partially unsaturated carbocyclylenyl, a 4-7 membered saturated or partially unsaturated heterocyclylenyl with 1-3 heteroatoms independently selected from nitrogen, oxygen, and sulfur, and a 5-6 membered heteroarylenyl with 1-4 heteroatoms independently selected from nitrogen, oxygen and sulfur;
Ring X is a ring selected from phenylenyl, a 4-7 membered saturated or partially unsaturated carbocyclylenyl, a 4-7 membered saturated or partially unsaturated heterocyclylenyl with 1-3 heteroatoms independently selected from nitrogen, oxygen, and sulfur, and a 5-6 membered heteroarylenyl with 1-4 heteroatoms independently selected from nitrogen, oxygen and sulfur;
each of L^{B}, L^{1′}, and L^{2′} is independently a covalent bond or a bivalent, saturated or partially unsaturated, straight or branched C₁₋₅ hydrocarbon chain, wherein 0-3 methylene units of L^{B}, L^{1′}, or L^{2′} are independently replaced by —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, or —OC(O)NR—, —NRC(O)O—;
each R^{x}, R^{y}, and R^{z} is independently selected from hydrogen, R^{B}, halogen, CN, —NO₂, —OR, —SR, —NR₂, —S(O)₂R, —S(O)₂NR₂, —S(O)R, —C(O)R, —C(O)OR, —C(O)NR₂, —C(O)NROR, —OC(O)R, —OC(O)NR₂, —NRC(O)OR, —NRC(O)R, —NRC(O)N(R)₂, and —NRS(O)₂R;
each R^{B} is independently an optionally substituted group selected from C₁₋₆ aliphatic, phenyl, a 3-12 membered saturated or partially unsaturated monocyclic, bicyclic, bridged bicyclic or spirocyclic carbocyclyl or heterocyclyl with 1-3 heteroatoms independently selected from nitrogen, oxygen, and sulfur, and a 5-6 membered heteroaryl ring having 1-4 heteroatoms independently selected from nitrogen, oxygen, and sulfur;
each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
two R groups on the same nitrogen are optionally taken together with their intervening atoms to form a 4-7 membered saturated, partially unsaturated, or heteroaryl ring having 0-3 heteroatoms, in addition to the nitrogen atom to which they are attached, independently selected from nitrogen, oxygen, and sulfur; and
x, y, and z are independently 0, 1, 2, 3, or 4; or
said compound is a compound of formula I-cc′: 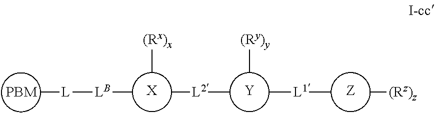
or a pharmaceutically acceptable salt thereof,
wherein;
Ring Z is a ring selected from phenyl, a 3-7 membered saturated or partially unsaturated monocyclic, bicyclic, bridged bicyclic or spirocyclic carbocyclic ring, a 3-7 membered saturated or partially unsaturated monocyclic, bicyclic, bridged bicyclic or spirocyclic heterocyclic ring with 1-3 heteroatoms independently selected from nitrogen, oxygen, and sulfur, and a 5-6 membered heteroaromatic ring with 1-4 heteroatoms independently selected from nitrogen, oxygen and sulfur and an 8-10 membered bicyclic heteroaromatic ring having 1-5 heteroatoms independently selected from nitrogen, oxygen, or sulfur;
Ring Y is a ring selected from phenylenyl, a 4-7 membered saturated or partially unsaturated carbocyclylenyl, a 4-8 membered saturated or partially unsaturated monocyclic, bicyclic, bridged bicyclic or spirocyclic heterocyclylenyl with 1-3 heteroatoms independently selected from nitrogen, oxygen, and sulfur, a 5-6 membered heteroarylenyl with 1-4 heteroatoms independently selected from nitrogen, oxygen and sulfur and an 8-10 membered bicyclic heterocyclylenyl having 1-5 heteroatoms independently selected from nitrogen, oxygen, or sulfur;
Ring X is a ring selected from phenylenyl, a 4-7 membered saturated or partially unsaturated carbocyclylenyl, a 4-7 membered saturated or partially unsaturated heterocyclylenyl with 1-3 heteroatoms independently selected from nitrogen, oxygen, and sulfur, and a 5-6 membered heteroarylenyl with 1-4 heteroatoms independently selected from nitrogen, oxygen and sulfur;
each of L^{B}, L^{1′}, and L^{2′} is independently a covalent bond or an optionally substituted bivalent, saturated or partially unsaturated, straight or branched C₁₋₅ hydrocarbon chain, wherein 0-3 methylene units of L^{B}, L^{1′}, or L^{2′} are independently replaced by -Cy′-, —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, or —OC(O)NR—, —NRC(O)O—;
each R^{x}, R^{y}, and R^{z} is independently selected from hydrogen, R^{B}, halogen, CN, —NO₂, —OR, —SR, —NR₂, —S(O)₂R, —S(O)₂NR₂, —S(O)R, —C(O)R, —C(O)OR, —C(O)NR₂, —C(O)NROR, —OC(O)R, —OC(O)NR₂, —NRC(O)OR, —NRC(O)R, —NRC(O)N(R)₂, and —NRS(O)₂R;
each R^{B} is independently an optionally substituted group selected from C₁₋₆ aliphatic, phenyl, a 3-12 membered saturated or partially unsaturated monocyclic, bicyclic, bridged bicyclic or spirocyclic carbocyclyl or heterocyclyl with 1-3 heteroatoms independently selected from nitrogen, oxygen, and sulfur, and a 5-6 membered heteroaryl ring having 1-4 heteroatoms independently selected from nitrogen, oxygen, and sulfur;
each -Cy′- is independently an optionally substituted bivalent ring selected from phenylenyl, an 8-10 membered bicyclic arylenyl, a 3-11 membered saturated or partially unsaturated monocyclic, bicyclic, bridged bicyclic or spirocyclic carbocyclylenyl, a 3-11 membered saturated or partially unsaturated monocyclic, bicyclic, bridged bicyclic or spirocyclic heterocyclylenyl having 1-3 heteroatoms independently selected from nitrogen, oxygen, and sulfur, a 5-6 membered heteroarylenyl having 1-4 heteroatoms independently selected from nitrogen, oxygen, and sulfur, and an 8-10 membered bicyclic heteroarylenyl having 1-5 heteroatoms independently selected from nitrogen, oxygen, and sulfur;
each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
two R groups on the same nitrogen are optionally taken together with their intervening atoms to form a 4-7 membered saturated, partially unsaturated, or heteroaryl ring having 0-3 heteroatoms, in addition to the nitrogen atom to which they are attached, independently selected from nitrogen, oxygen, and sulfur; and
x, y, and z are independently 0, 1, 2, 3, or 4; or
said compound is a compound of formula I-ff: 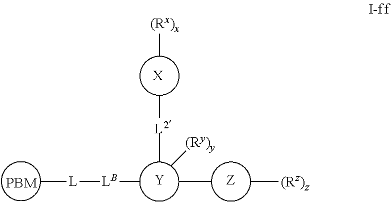
or a pharmaceutically acceptable salt thereof, wherein;
Ring Z is a ring selected from phenyl, a 4-7 membered saturated or partially unsaturated carbocyclic ring, a 4-7 membered saturated or partially unsaturated heterocyclic ring with 1-3 heteroatoms independently selected from nitrogen, oxygen, and sulfur, and a 5-6 membered heteroaromatic ring with 1-4 heteroatoms independently selected from nitrogen, oxygen and sulfur;
Ring Y is a ring selected from phenylenyl, a 4-7 membered saturated or partially unsaturated carbocyclylenyl, a 4-7 membered saturated or partially unsaturated heterocyclylenyl with 1-3 heteroatoms independently selected from nitrogen, oxygen, and sulfur, and a 5-6 membered heteroarylenyl with 1-4 heteroatoms independently selected from nitrogen, oxygen and sulfur;
Ring X is a ring selected from phenylenyl, a 4-7 membered saturated or partially unsaturated carbocyclylenyl, a 4-7 membered saturated or partially unsaturated heterocyclylenyl with 1-3 heteroatoms independently selected from nitrogen, oxygen, and sulfur, and a 5-6 membered heteroarylenyl with 1-4 heteroatoms independently selected from nitrogen, oxygen and sulfur;
each of L^{B} and L^{2′} is independently a covalent bond or a covalent bond or a bivalent, saturated or partially unsaturated, straight or branched C₁₋₅ hydrocarbon chain, wherein 0-3 methylene units of L^{B} or L^{2′} are independently replaced by —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, or —OC(O)NR—, —NRC(O)O—;
each R^{x}, R^{y}, and R^{z} is independently selected from hydrogen, R^{B}, halogen, —CN, —NO₂, —OR, —SR, —NR₂, —S(O)₂R, —S(O)₂NR₂, —S(O)R, —C(O)R, —C(O)OR, —C(O)NR₂, —C(O)NROR, —OC(O)R, —OC(O)NR₂, —NRC(O)OR, —NRC(O)R, —NRC(O)N(R)₂, and —NRS(O)₂R;
each R^{B} is independently an optionally substituted group selected from C₁₋₆ aliphatic, phenyl, a 3-12 membered saturated or partially unsaturated monocyclic, bicyclic, bridged bicyclic or spirocyclic carbocyclyl or heterocyclyl with 1-3 heteroatoms independently selected from nitrogen, oxygen, and sulfur, and a 5-6 membered heteroaryl ring having 1-4 heteroatoms independently selected from nitrogen, oxygen, and sulfur;
each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
two R groups on the same nitrogen are optionally taken together with their intervening atoms to form a 4-7 membered saturated, partially unsaturated, or heteroaryl ring having 0-3 heteroatoms, in addition to the nitrogen atom to which they are attached, independently selected from nitrogen, oxygen, and sulfur; and
x, y, and z are independently 0, 1, 2, 3, or 4; or
said compound is a compound of formula I-ff′: 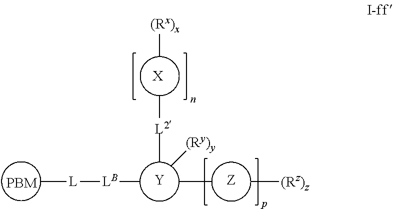
or a pharmaceutically acceptable salt thereof,
wherein;
Ring Z is a ring selected from phenyl, a 4-9 membered saturated or partially unsaturated monocyclic, bicyclic, bridged bicyclic or spirocyclic carbocyclic ring, a 4-9 membered saturated or partially unsaturated monocyclic, bicyclic, bridged bicyclic or spirocyclic heterocyclic ring with 1-3 heteroatoms independently selected from nitrogen, oxygen, and sulfur, a 5-6 membered heteroaromatic ring with 1-4 heteroatoms independently selected from nitrogen, oxygen and sulfur and an 8-10 membered bicyclic heteroaromatic ring having 1-5 heteroatoms independently selected from nitrogen, oxygen, or sulfur;
Ring Y is a ring selected from phenylenyl, a 4-7 membered saturated or partially unsaturated carbocyclylenyl, a 4-7 membered saturated or partially unsaturated heterocyclylenyl with 1-3 heteroatoms independently selected from nitrogen, oxygen, and sulfur, and a 5-6 membered heteroarylenyl with 1-4 heteroatoms independently selected from nitrogen, oxygen and sulfur;
Ring X is a ring selected from phenylenyl, a 3-9 membered saturated or partially unsaturated monocyclic, bicyclic, bridged bicyclic or spirocyclic carbocyclylenyl, a 3-9 membered saturated or partially unsaturated monocyclic, bicyclic, bridged bicyclic or spirocyclic heterocyclylenyl with 1-3 heteroatoms independently selected from nitrogen, oxygen, and sulfur, and a 5-6 membered heteroarylenyl with 1-4 heteroatoms independently selected from nitrogen, oxygen and sulfur;
each of L^{B} and L^{2′} is independently a covalent bond or an optionally substituted bivalent, saturated or partially unsaturated, straight or branched C₁₋₅ hydrocarbon chain, wherein 0-3 methylene units of L^{B} or L^{2′} are independently replaced by -Cy′-, —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, or —OC(O)NR—, —NRC(O)O—;
each R^{x}, R^{y}, and R^{z} is independently selected from hydrogen, R^{B}, halogen, —CN, —NO₂, —OR, —SR, —NR₂, —S(O)₂R, —S(O)₂NR₂, —S(O)R, —C(O)R, —C(O)OR, —C(O)NR₂, —C(O)NROR, —OC(O)R, —OC(O)NR₂, —NRC(O)OR, —NRC(O)R, —NRC(O)N(R)₂, and —NRS(O)₂R;
each R^{B} is independently an optionally substituted group selected from C₁₋₆ aliphatic, phenyl, a 3-12 membered saturated or partially unsaturated monocyclic, bicyclic, bridged bicyclic or spirocyclic carbocyclyl or heterocyclyl with 1-3 heteroatoms independently selected from nitrogen, oxygen, and sulfur, and a 5-6 membered heteroaryl ring having 1-4 heteroatoms independently selected from nitrogen, oxygen, and sulfur;
each -Cy′- is independently an optionally substituted bivalent ring selected from phenylenyl, an 8-10 membered bicyclic arylenyl, a 3-11 membered saturated or partially unsaturated monocyclic, bicyclic, bridged bicyclic or spirocyclic carbocyclylenyl, a 3-11 membered saturated or partially unsaturated monocyclic, bicyclic, bridged bicyclic or spirocyclic heterocyclylenyl having 1-3 heteroatoms independently selected from nitrogen, oxygen, and sulfur, a 5-6 membered heteroarylenyl having 1-4 heteroatoms independently selected from nitrogen, oxygen, and sulfur, and an 8-10 membered bicyclic heteroarylenyl having 1-5 heteroatoms independently selected from nitrogen, oxygen, and sulfur;
each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
two R groups on the same nitrogen are optionally taken together with their intervening atoms to form a 4-7 membered saturated, partially unsaturated, or heteroaryl ring having 0-3 heteroatoms, in addition to the nitrogen atom to which they are attached, independently selected from nitrogen, oxygen, and sulfur;
n and p are independently 0 or 1; and
x, y, and z are independently 0, 1, 2, 3, or 4; or
said compound is a compound of formula I-gg: 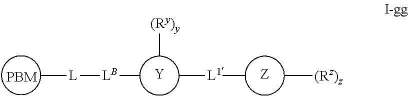
or a pharmaceutically acceptable salt thereof,
wherein:
Ring Z is a ring selected from phenyl, a 4-7 membered saturated or partially unsaturated carbocyclic ring, a 4-7 membered saturated or partially unsaturated heterocyclic ring with 1-3 heteroatoms independently selected from nitrogen, oxygen, and sulfur, and a 5-6 membered heteroaromatic ring with 1-4 heteroatoms independently selected from nitrogen, oxygen and sulfur;
Ring Y is a ring selected from phenylenyl, a 4-7 membered saturated or partially unsaturated carbocyclylenyl, a 4-7 membered saturated or partially unsaturated heterocyclylenyl with 1-3 heteroatoms independently selected from nitrogen, oxygen, and sulfur, and a 5-6 membered heteroarylenyl with 1-4 heteroatoms independently selected from nitrogen, oxygen and sulfur;
each of L^{B} and L^{1′} is independently a covalent bond or a covalent bond or a bivalent, saturated or partially unsaturated, straight or branched C₁₋₅ hydrocarbon chain, wherein 0-3 methylene units of L^{B} or L^{1′} are independently replaced by —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, or —OC(O)NR—, —NRC(O)O—;
each R^{y}, and R^{z} is independently selected from hydrogen, R^{B}, halogen, CN, —NO₂, —OR, —SR, —NR₂, —S(O)₂R, —S(O)₂NR₂, —S(O)R, —C(O)R, —C(O)OR, —C(O)NR₂, —C(O)NROR, —OC(O)R, —OC(O)NR₂, —NRC(O)OR, —NRC(O)R, —NRC(O)N(R)₂, and —NRS(O)₂R;
each R^{B} is independently an optionally substituted group selected from C₁₋₆ aliphatic, phenyl, a 3-12 membered saturated or partially unsaturated monocyclic, bicyclic, bridged bicyclic or spirocyclic carbocyclyl or heterocyclyl with 1-3 heteroatoms independently selected from nitrogen, oxygen, and sulfur, and a 5-6 membered heteroaryl ring having 1-4 heteroatoms independently selected from nitrogen, oxygen, and sulfur;
each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
two R groups on the same nitrogen are optionally taken together with their intervening atoms to form a 4-7 membered saturated, partially unsaturated, or heteroaryl ring having 0-3 heteroatoms, in addition to the nitrogen atom to which they are attached, independently selected from nitrogen, oxygen, and sulfur; and
y and z are independently 0, 1, 2, 3, or 4; or
said compound is a compound of formula I-hh: 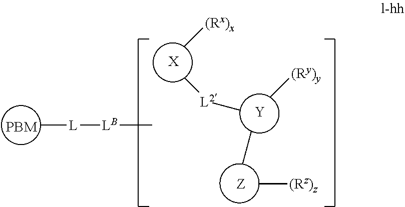
or a pharmaceutically acceptable salt thereof,
wherein;
Ring Z is a ring selected from phenyl, a 4-7 membered saturated or partially unsaturated carbocyclic ring, a 4-7 membered saturated or partially unsaturated heterocyclic ring with 1-3 heteroatoms independently selected from nitrogen, oxygen, and sulfur, a 5-6 membered heteroaromatic ring with 1-4 heteroatoms independently selected from nitrogen, oxygen and sulfur, and an 8-10 membered bicyclic heteroaromatic ring having 1-5 heteroatoms independently selected from nitrogen, oxygen, or sulfur;
Ring Y is a ring selected from phenylenyl, a 4-7 membered saturated or partially unsaturated carbocyclylenyl, a 4-7 membered saturated or partially unsaturated heterocyclylenyl with 1-3 heteroatoms independently selected from nitrogen, oxygen, and sulfur, and a 5-6 membered heteroarylenyl with 1-4 heteroatoms independently selected from nitrogen, oxygen and sulfur;
Ring X is a ring selected from phenylenyl, a 4-7 membered saturated or partially unsaturated carbocyclylenyl, a 4-7 membered saturated or partially unsaturated heterocyclylenyl with 1-3 heteroatoms independently selected from nitrogen, oxygen, and sulfur, and a 5-6 membered heteroarylenyl with 1-4 heteroatoms independently selected from nitrogen, oxygen and sulfur;
each L^{B} and L^{2′} is independently a covalent bond or a bivalent, saturated or partially unsaturated, straight or branched C₁₋₅ hydrocarbon chain, wherein 0-3 methylene units of L^{B} or L^{2′} are independently replaced by —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, or —OC(O)NR—, —NRC(O)O—;
each R^{x}, R^{y}, and R^{z} is independently selected from hydrogen, R^{B}, halogen, CN, —NO₂, —OR, —SR, —NR₂, —S(O)₂R, —S(O)₂NR₂, —S(O)R, —C(O)R, —C(O)OR, —C(O)NR₂, —C(O)NROR, —OC(O)R, —OC(O)NR₂, —NRC(O)OR, —NRC(O)R, —NRC(O)N(R)₂, and —NRS(O)₂R;
each R^{B} is independently an optionally substituted group selected from C₁₋₆ aliphatic, phenyl, a 3-12 membered saturated or partially unsaturated monocyclic, bicyclic, bridged bicyclic or spirocyclic carbocyclyl or heterocyclyl with 1-3 heteroatoms independently selected from nitrogen, oxygen, and sulfur, and a 5-6 membered heteroaryl ring having 1-4 heteroatoms independently selected from nitrogen, oxygen, and sulfur;
each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
two R groups on the same nitrogen are optionally taken together with their intervening atoms to form a 4-7 membered saturated, partially unsaturated, or heteroaryl ring having 0-3 heteroatoms, in addition to the nitrogen atom to which they are attached, independently selected from nitrogen, oxygen, and sulfur; and
x, y, and z are independently 0, 1, 2, 3, or 4; or
said compound is a compound of formula I-ii: 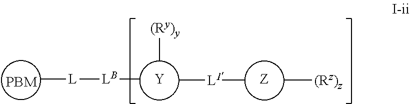
or a pharmaceutically acceptable salt thereof,
wherein;
Ring Y is a ring selected from phenylenyl, a 4-7 membered saturated or partially unsaturated carbocyclylenyl, a 4-7 membered saturated or partially unsaturated heterocyclylenyl with 1-3 heteroatoms independently selected from nitrogen, oxygen, and sulfur, a 5-6 membered heteroarylenyl with 1-4 heteroatoms independently selected from nitrogen, oxygen and sulfur and an 8-10 membered bicyclic heterocyclylenyl having 1-5 heteroatoms independently selected from nitrogen, oxygen, or sulfur;
each L^{B} and L^{1′} is a covalent bond or a bivalent, saturated or partially unsaturated, straight or branched C₁₋₅ hydrocarbon chain, wherein 0-3 methylene units of L^{B} or L^{1′} are independently replaced by —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, or —OC(O)NR—, —NRC(O)O—;
Ring Z is a ring selected from phenyl, a 4-7 membered saturated or partially unsaturated carbocyclic ring, a 4-7 membered saturated or partially unsaturated heterocyclic ring with 1-3 heteroatoms independently selected from nitrogen, oxygen, and sulfur, and a 5-6 membered heteroaromatic ring with 1-4 heteroatoms independently selected from nitrogen, oxygen and sulfur;
each R^{y}, and R^{z} is independently selected from hydrogen, R^{B}, halogen, CN, —NO₂, —OR, —SR, —NR₂, —S(O)₂R, —S(O)₂NR₂, —S(O)R, —C(O)R, —C(O)OR, —C(O)NR₂, —C(O)NROR, —OC(O)R, —OC(O)NR₂, —NRC(O)OR, —NRC(O)R, —NRC(O)N(R)₂, and —NRS(O)₂R;
each R^{B} is independently an optionally substituted group selected from C₁₋₆ aliphatic, phenyl, a 3-12 membered saturated or partially unsaturated monocyclic, bicyclic, bridged bicyclic or spirocyclic carbocyclyl or heterocyclyl with 1-3 heteroatoms independently selected from nitrogen, oxygen, and sulfur, and a 5-6 membered heteroaryl ring having 1-4 heteroatoms independently selected from nitrogen, oxygen, and sulfur;
each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
two R groups on the same nitrogen are optionally taken together with their intervening atoms to form a 4-7 membered saturated, partially unsaturated, or heteroaryl ring having 0-3 heteroatoms, in addition to the nitrogen atom to which they are attached, independently selected from nitrogen, oxygen, and sulfur; and
y, and z are independently 0, 1, 2, 3, or 4; or
said compound is a compound of formula I-kk: 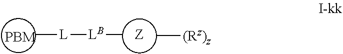
or a pharmaceutically acceptable salt thereof,
wherein;
Ring Z is a ring selected from phenyl, a 4-7 membered saturated or partially unsaturated carbocyclic ring, an 8-10 membered bicyclic aromatic carbocyclic ring, a 4-8 membered saturated or partially unsaturated monocyclic heterocyclic ring having 1-2 heteroatoms independently selected from nitrogen, oxygen, or sulfur, a 5-6 membered monocyclic heteroaromatic ring having 1-4 heteroatoms independently selected from nitrogen, oxygen, or sulfur, and an 8-10 membered bicyclic heteroaromatic ring having 1-5 heteroatoms independently selected from nitrogen, oxygen, or sulfur;
each R^{z} is independently selected from hydrogen, R^{B}, halogen, CN, —NO₂, —OR, —SR, —NR₂, —S(O)₂R, —S(O)₂NR₂, —S(O)R, —C(O)R, —C(O)OR, —C(O)NR₂, —C(O)NROR, —OC(O)R, —OC(O)NR₂, —NRC(O)OR, —NRC(O)R, —NRC(O)N(R)₂, and —NRS(O)₂R;
L^{B} is a covalent bond or a bivalent, saturated or partially unsaturated, straight or branched C₁₋₅ hydrocarbon chain, wherein 0-3 methylene units of L^{B} are independently replaced by —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, or —OC(O)NR—, —NRC(O)O—;
each R^{B} is independently an optionally substituted group selected from C₁₋₆ aliphatic, phenyl, a 3-12 membered saturated or partially unsaturated monocyclic, bicyclic, bridged bicyclic or spirocyclic carbocyclyl or heterocyclyl with 1-3 heteroatoms independently selected from nitrogen, oxygen, and sulfur, and a 5-6 membered heteroaryl ring having 1-4 heteroatoms independently selected from nitrogen, oxygen, and sulfur;
each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
two R groups on the same nitrogen are optionally taken together with their intervening atoms to form a 4-7 membered saturated, partially unsaturated, or heteroaryl ring having 0-3 heteroatoms, in addition to the nitrogen atom to which they are attached, independently selected from nitrogen, oxygen, and sulfur; and
z is 0, 1, 2, 3, or 4; or
said compound is a compound of formula I-11: 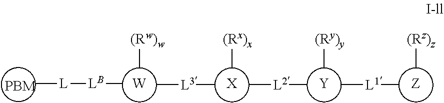
or a pharmaceutically acceptable salt thereof,
wherein;
Ring Z is a ring selected from phenyl, a 4-7 membered saturated or partially unsaturated carbocyclic ring, a 4-7 membered saturated or partially unsaturated heterocyclic ring with 1-3 heteroatoms independently selected from nitrogen, oxygen, and sulfur, and a 5-6 membered heteroaromatic ring with 1-4 heteroatoms independently selected from nitrogen, oxygen and sulfur;
Ring Y is a ring selected from phenylenyl, a 4-7 membered saturated or partially unsaturated carbocyclylenyl, a 4-7 membered saturated or partially unsaturated heterocyclylenyl with 1-3 heteroatoms independently selected from nitrogen, oxygen, and sulfur, and a 5-6 membered heteroarylenyl with 1-4 heteroatoms independently selected from nitrogen, oxygen and sulfur;
Ring X is a ring selected from phenylenyl, a 4-7 membered saturated or partially unsaturated carbocyclylenyl, a 4-7 membered saturated or partially unsaturated heterocyclylenyl with 1-3 heteroatoms independently selected from nitrogen, oxygen, and sulfur, and a 5-6 membered heteroarylenyl with 1-4 heteroatoms independently selected from nitrogen, oxygen and sulfur;
Ring W is a ring selected from phenylenyl, a 4-7 membered saturated or partially unsaturated carbocyclylenyl, a 4-7 membered saturated or partially unsaturated heterocyclylenyl with 1-3 heteroatoms independently selected from nitrogen, oxygen, and sulfur, and a 5-6 membered heteroarylenyl with 1-4 heteroatoms independently selected from nitrogen, oxygen and sulfur;
each L^{B}, L^{1′}, and L^{3′} is independently a covalent bond or a bivalent, saturated or partially unsaturated, straight or branched C₁₋₅ hydrocarbon chain, wherein 0-3 methylene units of L^{B}, L^{1′}, or L^{3′} are independently replaced by —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, or —OC(O)NR—, —NRC(O)O—;
each R^{w}, R^{x}, R^{y}, and R^{z} is independently selected from hydrogen, R^{B}, halogen, —CN, —NO₂, —OR, —SR, —NR₂, —S(O)₂R, —S(O)₂NR₂, —S(O)R, —C(O)R, —C(O)OR, —C(O)NR₂, —C(O)NROR, —OC(O)R, —OC(O)NR₂, —NRC(O)OR, —NRC(O)R, —NRC(O)N(R)₂, and —NRS(O)₂R;
each R^{B} is independently an optionally substituted group selected from C₁₋₆ aliphatic, phenyl, a 3-12 membered saturated or partially unsaturated monocyclic, bicyclic, bridged bicyclic or spirocyclic carbocyclyl or heterocyclyl with 1-3 heteroatoms independently selected from nitrogen, oxygen, and sulfur, and a 5-6 membered heteroaryl ring having 1-4 heteroatoms independently selected from nitrogen, oxygen, and sulfur;
each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
two R groups on the same nitrogen are optionally taken together with their intervening atoms to form a 4-7 membered saturated, partially unsaturated, or heteroaryl ring having 0-3 heteroatoms, in addition to the nitrogen atom to which they are attached, independently selected from nitrogen, oxygen, and sulfur; and
w, x, y, and z are independently 0, 1, 2, 3, or 4; or
said compound is a compound of formula I-11′: 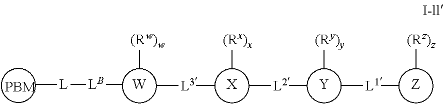
or a pharmaceutically acceptable salt thereof,
wherein:
Ring Z is a ring selected from phenylenyl, a 3-7 membered saturated or partially unsaturated monocyclic, bicyclic, bridged bicyclic or spirocyclic carbocyclylenyl, a 3-7 membered saturated or partially unsaturated monocyclic, bicyclic, bridged bicyclic or spirocyclic heterocyclylenyl with 1-3 heteroatoms independently selected from nitrogen, oxygen, and sulfur, and a 5-6 membered heteroarylenyl with 1-4 heteroatoms independently selected from nitrogen, oxygen and sulfur;
Ring Y is a ring selected from phenylenyl, a 4-7 membered saturated or partially unsaturated carbocyclylenyl, a 4-7 membered saturated or partially unsaturated heterocyclylenyl with 1-3 heteroatoms independently selected from nitrogen, oxygen, and sulfur, and a 5-6 membered heteroarylenyl with 1-4 heteroatoms independently selected from nitrogen, oxygen and sulfur;
Ring X is a ring selected from phenylenyl, a 4-7 membered saturated or partially unsaturated carbocyclylenyl, a 4-7 membered saturated or partially unsaturated heterocyclylenyl with 1-3 heteroatoms independently selected from nitrogen, oxygen, and sulfur, and a 5-6 membered heteroarylenyl with 1-4 heteroatoms independently selected from nitrogen, oxygen and sulfur;
Ring W is a ring selected from phenylenyl, a 4-7 membered saturated or partially unsaturated carbocyclylenyl, a 4-7 membered saturated or partially unsaturated heterocyclylenyl with 1-3 heteroatoms independently selected from nitrogen, oxygen, and sulfur, and a 5-6 membered heteroarylenyl with 1-4 heteroatoms independently selected from nitrogen, oxygen and sulfur;
each L^{B}, L^{1′}, and L^{3′} is independently a covalent bond or an optionally substituted bivalent, saturated or partially unsaturated, straight or branched C₁₋₅ hydrocarbon chain, wherein 0-3 methylene units of L^{B}, L^{1′}, or L^{3′} are independently replaced by —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, or —OC(O)NR—, —NRC(O)O—;
each R^{w}, R^{x}, R^{y}, and R^{z} is independently selected from hydrogen, R^{B}, halogen, —CN, —NO₂, —OR, —SR, —NR₂, —S(O)₂R, —S(O)₂NR₂, —S(O)R, —C(O)R, —C(O)OR, —C(O)NR₂, —C(O)NROR, —OC(O)R, —OC(O)NR₂, —NRC(O)OR, —NRC(O)R, —NRC(O)N(R)₂, and —NRS(O)₂R;
each R^{B} is independently an optionally substituted group selected from C₁₋₆ aliphatic, phenyl, a 3-12 membered saturated or partially unsaturated monocyclic, bicyclic, bridged bicyclic or spirocyclic carbocyclyl or heterocyclyl with 1-3 heteroatoms independently selected from nitrogen, oxygen, and sulfur, and a 5-6 membered heteroaryl ring having 1-4 heteroatoms independently selected from nitrogen, oxygen, and sulfur;
each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
two R groups on the same nitrogen are optionally taken together with their intervening atoms to form a 4-7 membered saturated, partially unsaturated, or heteroaryl ring having 0-3 heteroatoms, in addition to the nitrogen atom to which they are attached, independently selected from nitrogen, oxygen, and sulfur:
p is independently 0 or 1; and
w, x, y, and z are independently 0, 1, 2, 3, or 4.

17-27. (canceled)

28. The compound of claim 16, wherein Ring W is cyclohexyl.

29. The compound of claim 16, wherein Ring X is a phenylenyl.

30-31. (canceled)

32. The compound of claim 16, wherein Ring Y is phenylenyl,

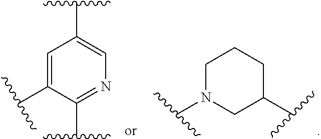 [SMILES: *c1cnc(*)c(*)c1 ; *C1CCCN(*)C1]

33-34. (canceled)

35. The compound of claim 16, wherein Ring Z is a phenyl or a cyclopropyl.

36-38. (canceled)

39. The compound of claim 16, of any of the following formulae: 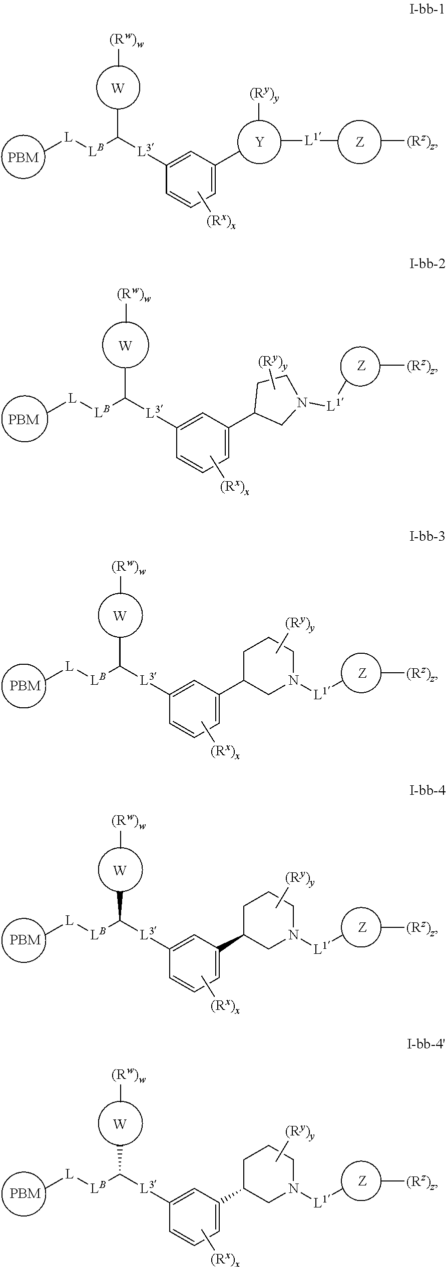 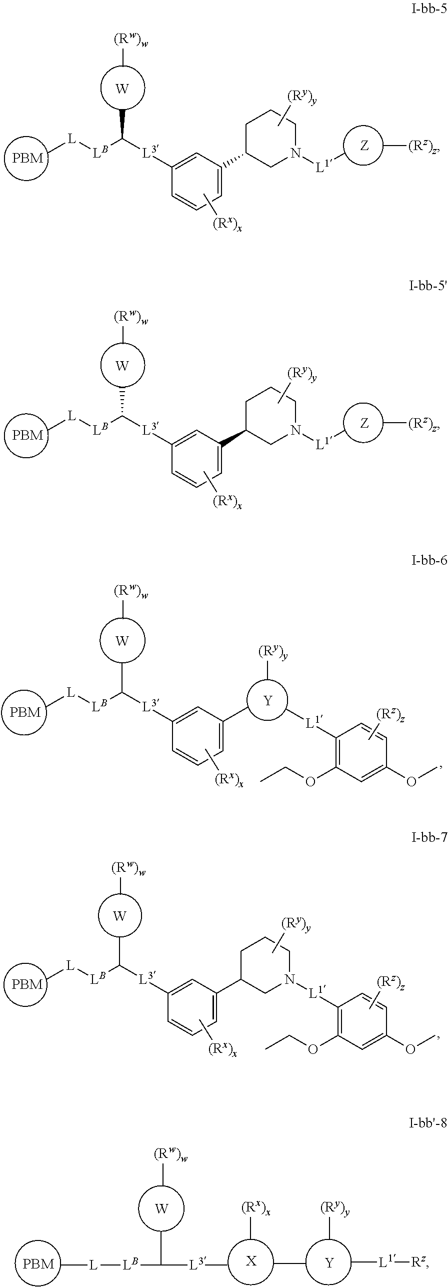 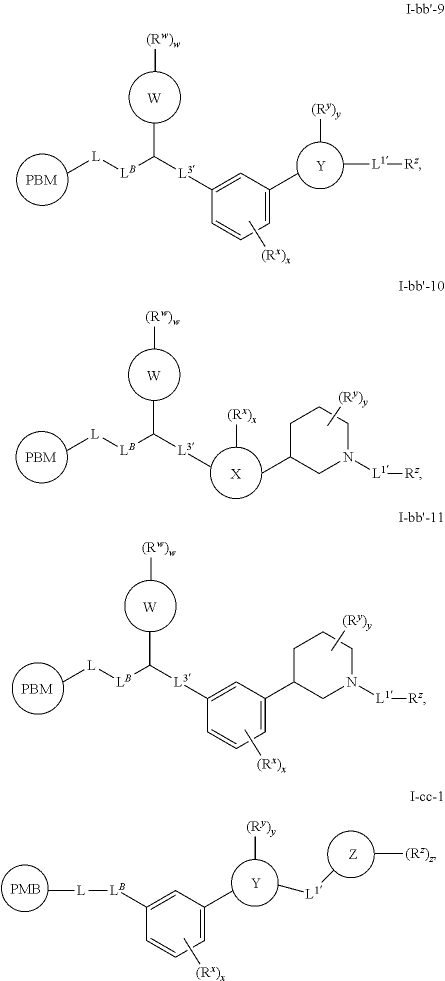 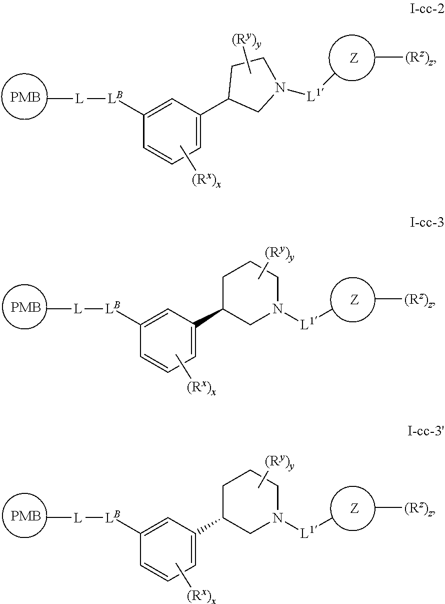 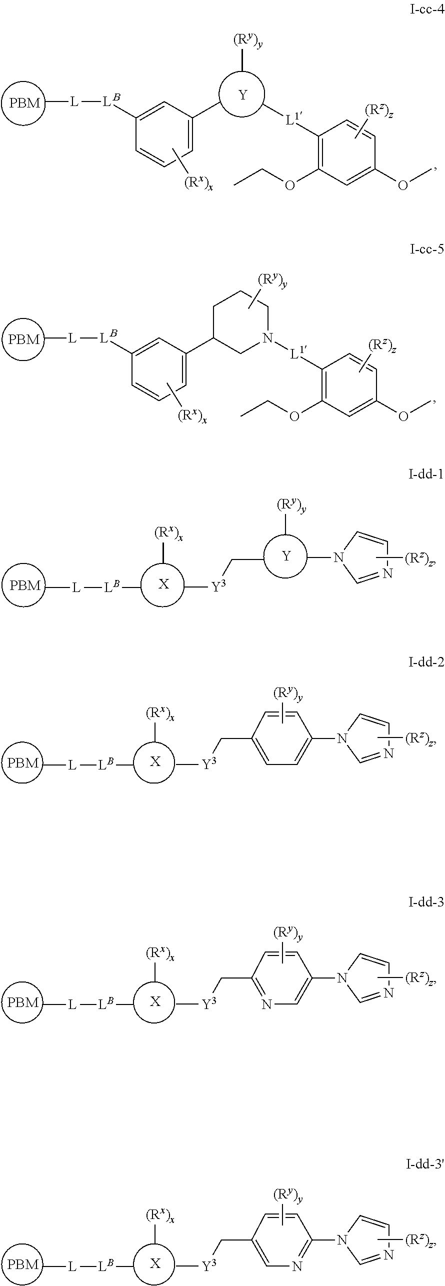 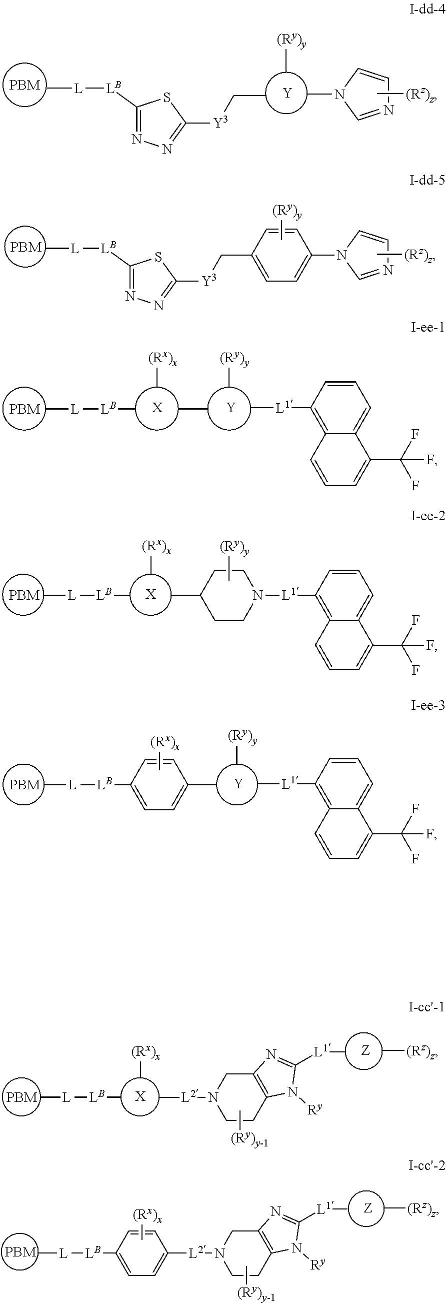 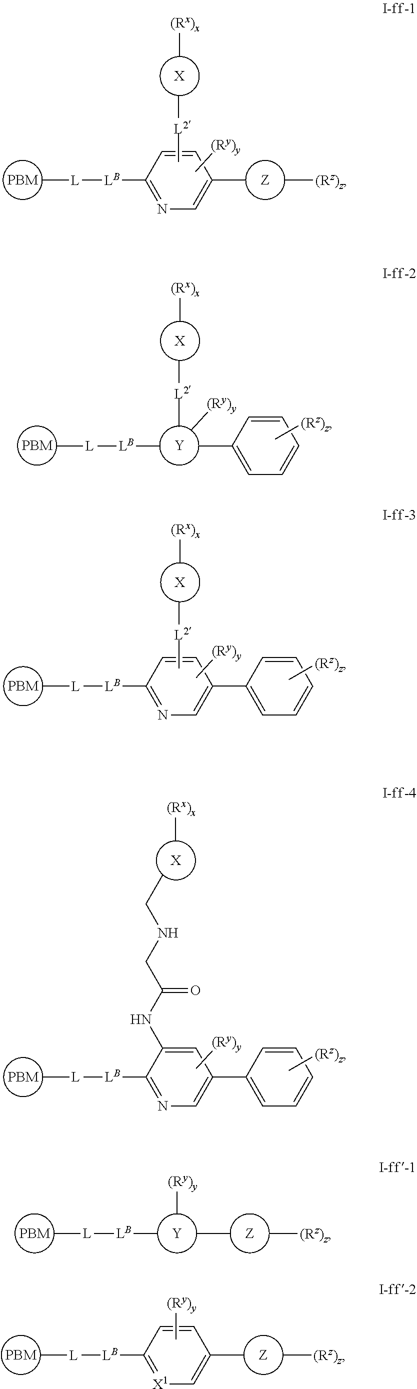 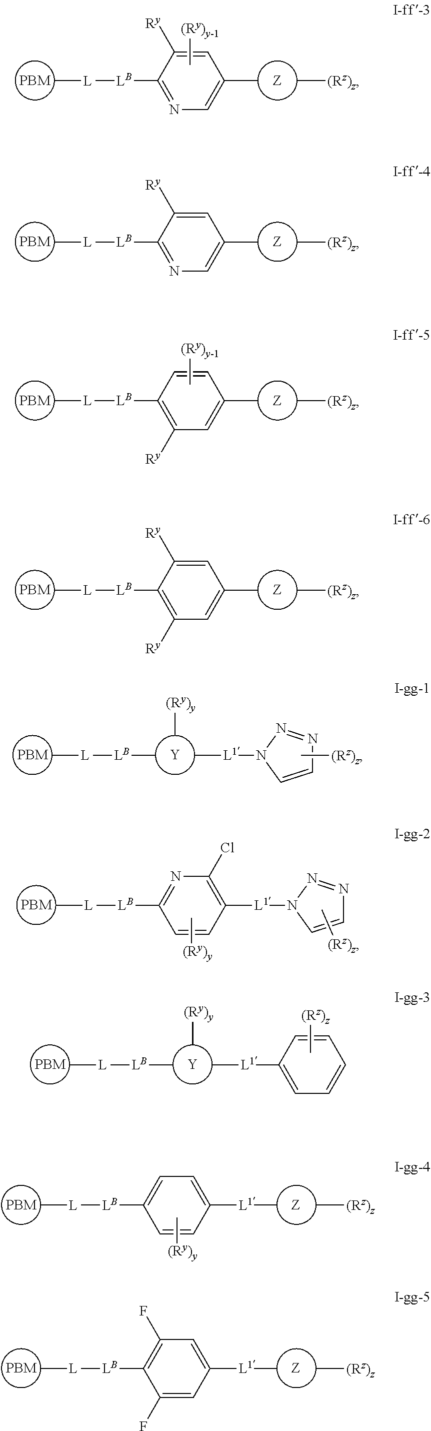 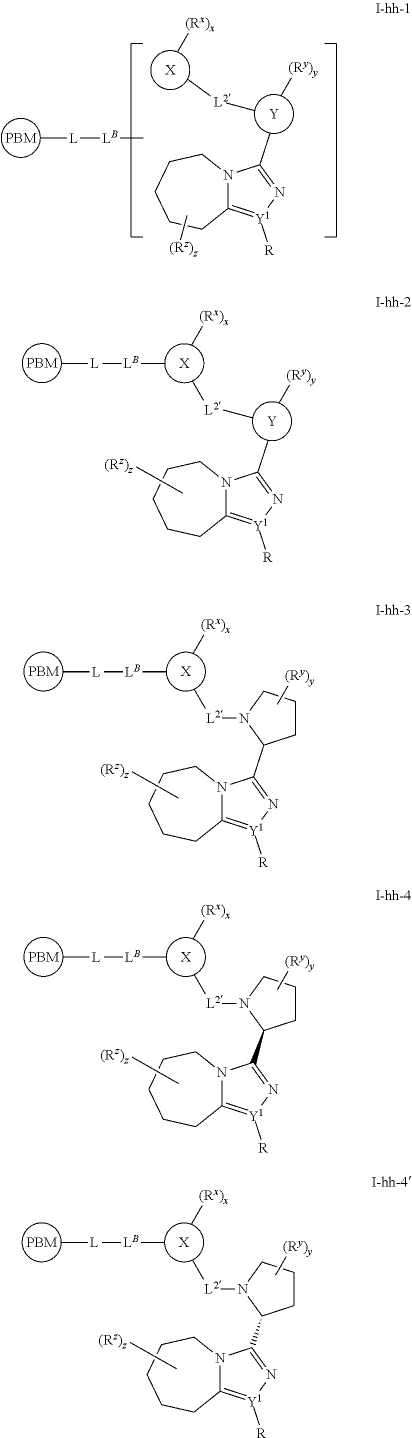 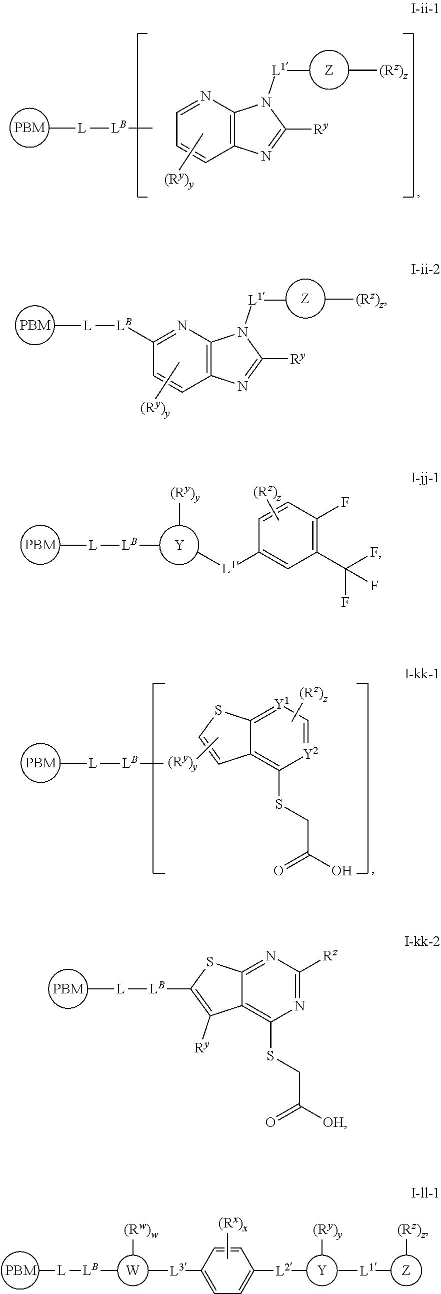 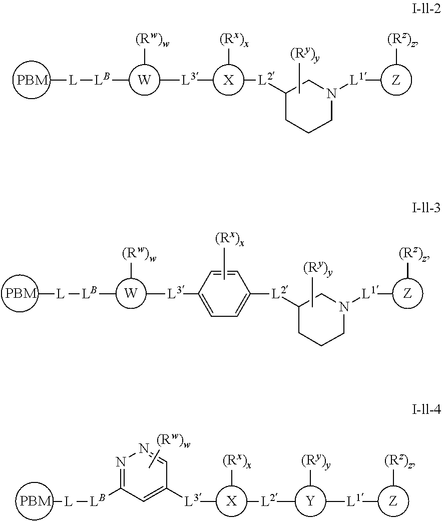
or pharmaceutically acceptable salt thereof.

40-47. (canceled)

48. The compound of claim 1, wherein the PBM is a PARP binding moiety selected from

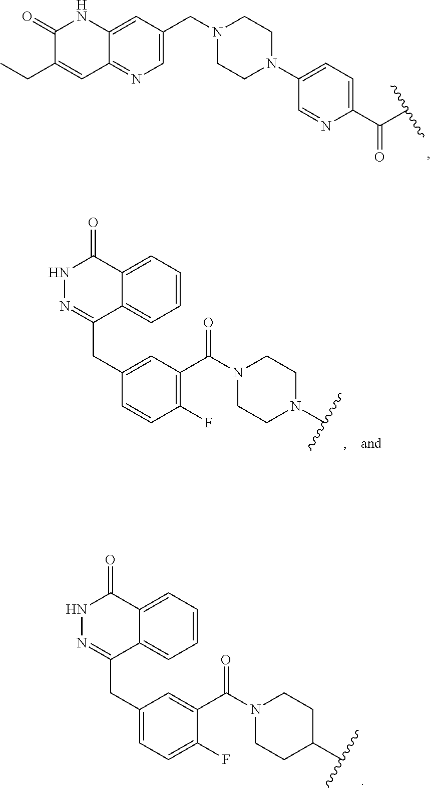 [SMILES: *C(=O)c1ccc(N2CCN(Cc3cnc4cc(CC)c(=O)[nH]c4c3)CC2)cn1 ; *N1CCN(C(=O)c2cc(Cc3n[nH]c(=O)c4ccccc34)ccc2F)CC1 ; *C1CCN(C(=O)c2cc(Cc3n[nH]c(=O)c4ccccc34)ccc2F)CC1]

49. The compound of claim 1, wherein the DIM is a deubiquitinase binding moiety selected from:

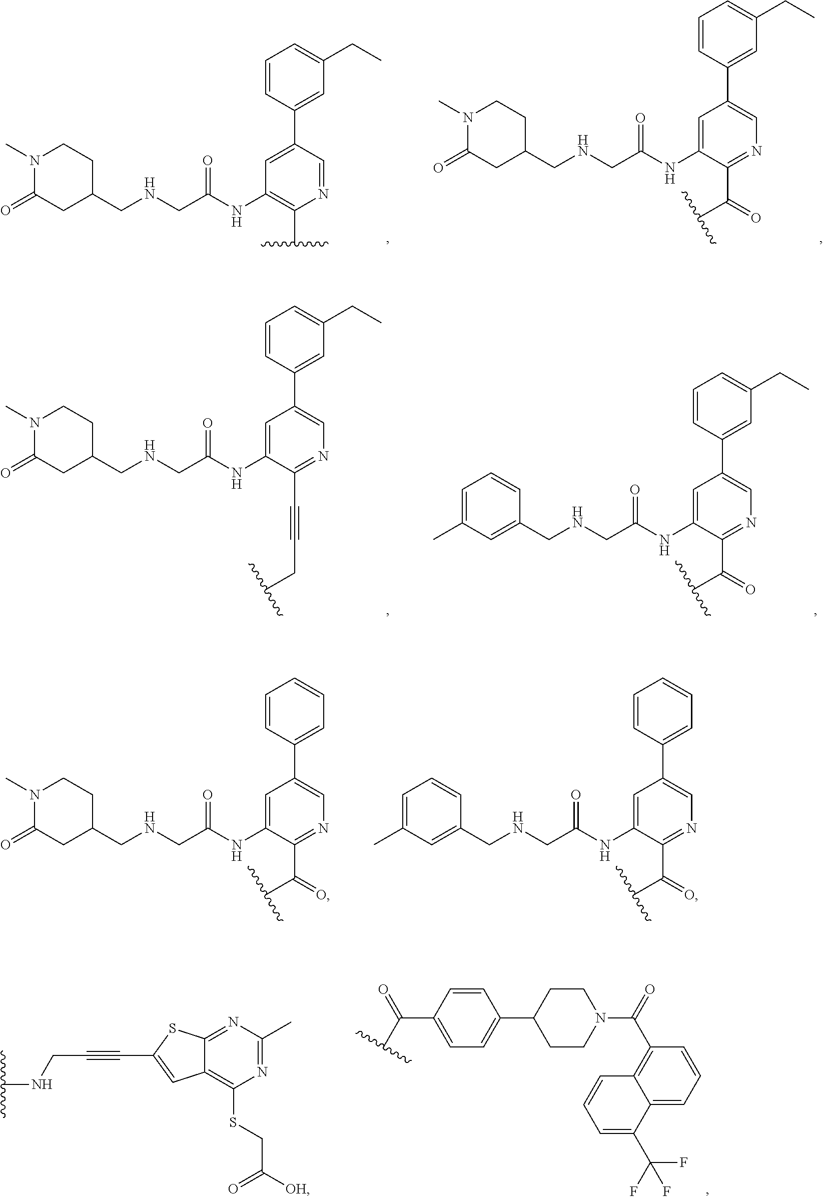

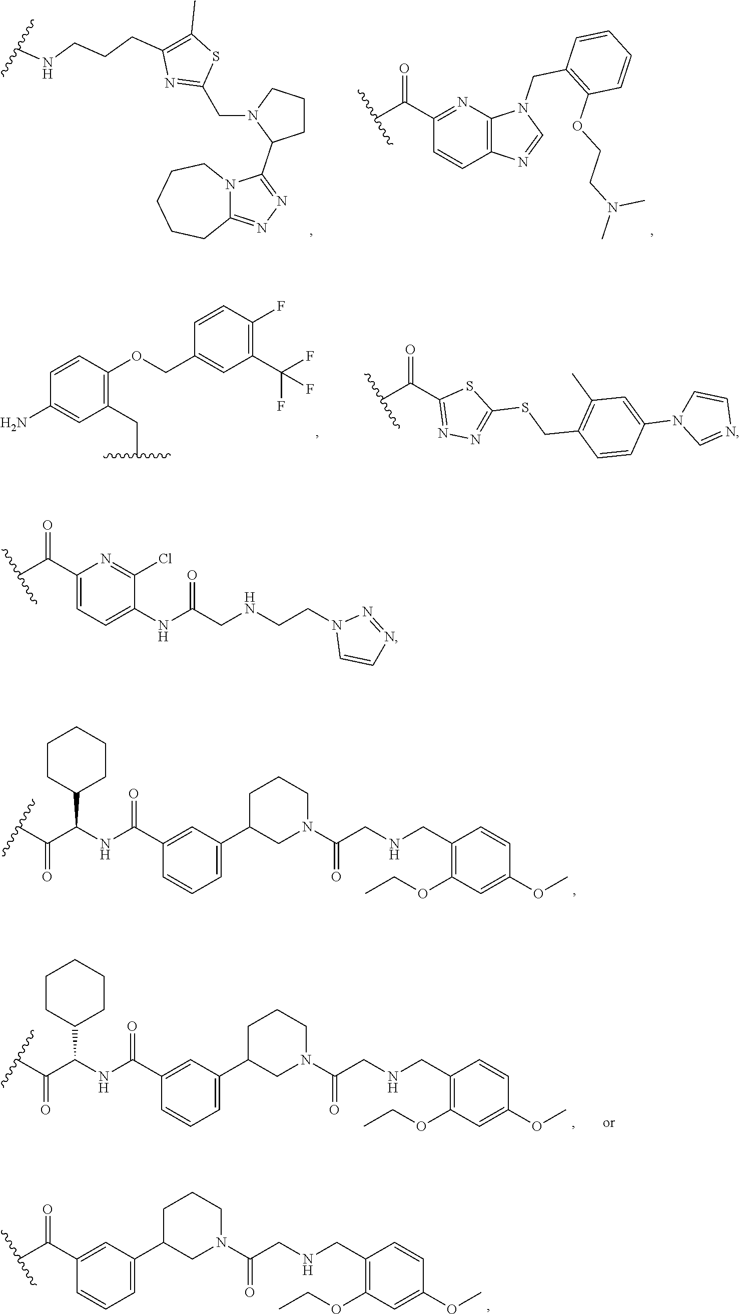

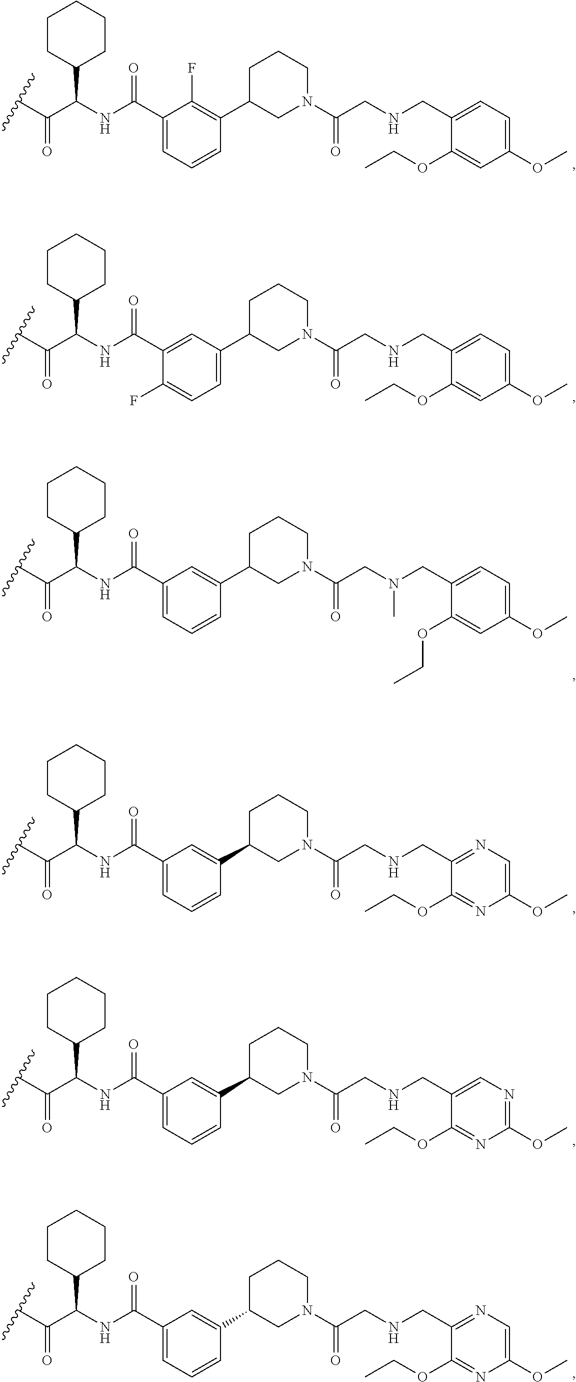 [SMILES: *C(=O)[C@H](NC(=O)c1cccc(C2CCCN(C(=O)CNCc3ccc(OC)cc3OCC)C2)c1F)C1CCCCC1 ; *C(=O)[C@H](NC(=O)c1cc(C2CCCN(C(=O)CNCc3ccc(OC)cc3OCC)C2)ccc1F)C1CCCCC1 ; *C(=O)[C@H](NC(=O)c1cccc(C2CCCN(C(=O)CN(C)Cc3ccc(OC)cc3OCC)C2)c1)C1CCCCC1 ; *C(=O)[C@H](NC(=O)c1cccc([C@H]2CCCN(C(=O)CNCc3ncc(OC)nc3OCC)C2)c1)C1CCCCC1 ; *C(=O)[C@H](NC(=O)c1cccc([C@H]2CCCN(C(=O)CNCc3cnc(OC)nc3OCC)C2)c1)C1CCCCC1 ; *C(=O)[C@H](NC(=O)c1cccc([C@@H]2CCCN(C(=O)CNCc3ncc(OC)nc3OCC)C2)c1)C1CCCCC1]

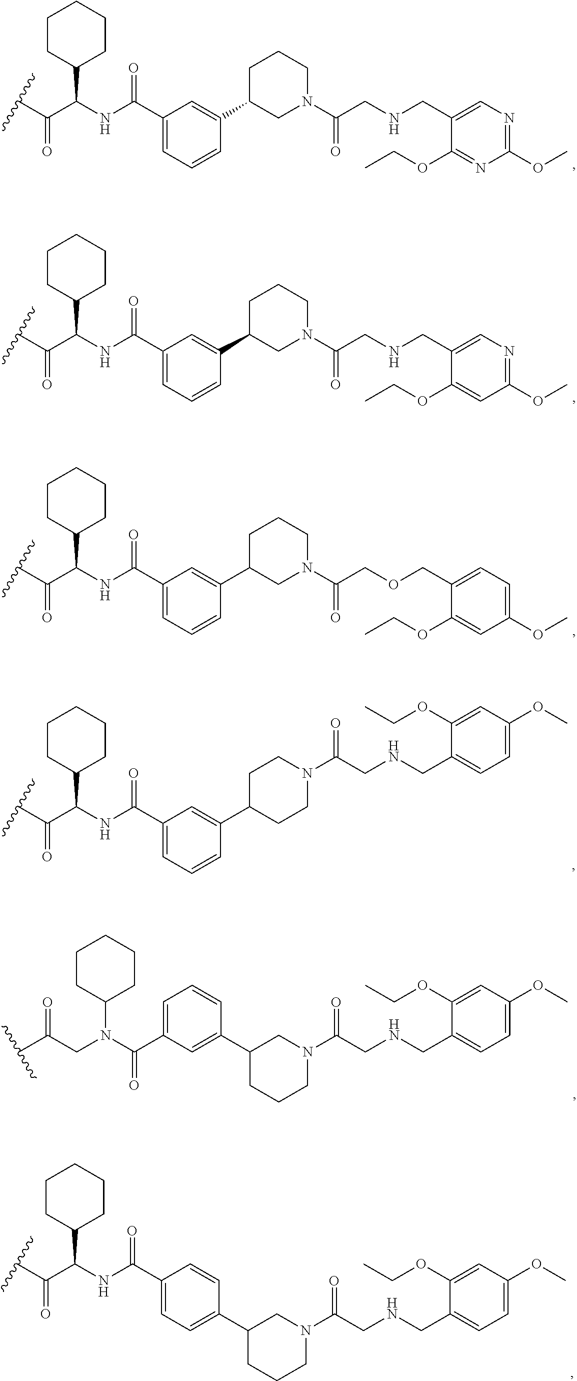 [SMILES: *C(=O)[C@H](NC(=O)c1cccc([C@@H]2CCCN(C(=O)CNCc3cnc(OC)nc3OCC)C2)c1)C1CCCCC1 ; *C(=O)[C@H](NC(=O)c1cccc([C@H]2CCCN(C(=O)CNCc3cnc(OC)cc3OCC)C2)c1)C1CCCCC1 ; *C(=O)[C@H](NC(=O)c1cccc(C2CCCN(C(=O)COCc3ccc(OC)cc3OCC)C2)c1)C1CCCCC1 ; *C(=O)[C@H](NC(=O)c1cccc(C2CCN(C(=O)CNCc3ccc(OC)cc3OCC)CC2)c1)C1CCCCC1 ; *C(=O)CN(C(=O)c1cccc(C2CCCN(C(=O)CNCc3ccc(OC)cc3OCC)C2)c1)C1CCCCC1 ; *C(=O)[C@H](NC(=O)c1ccc(C2CCCN(C(=O)CNCc3ccc(OC)cc3OCC)C2)cc1)C1CCCCC1]

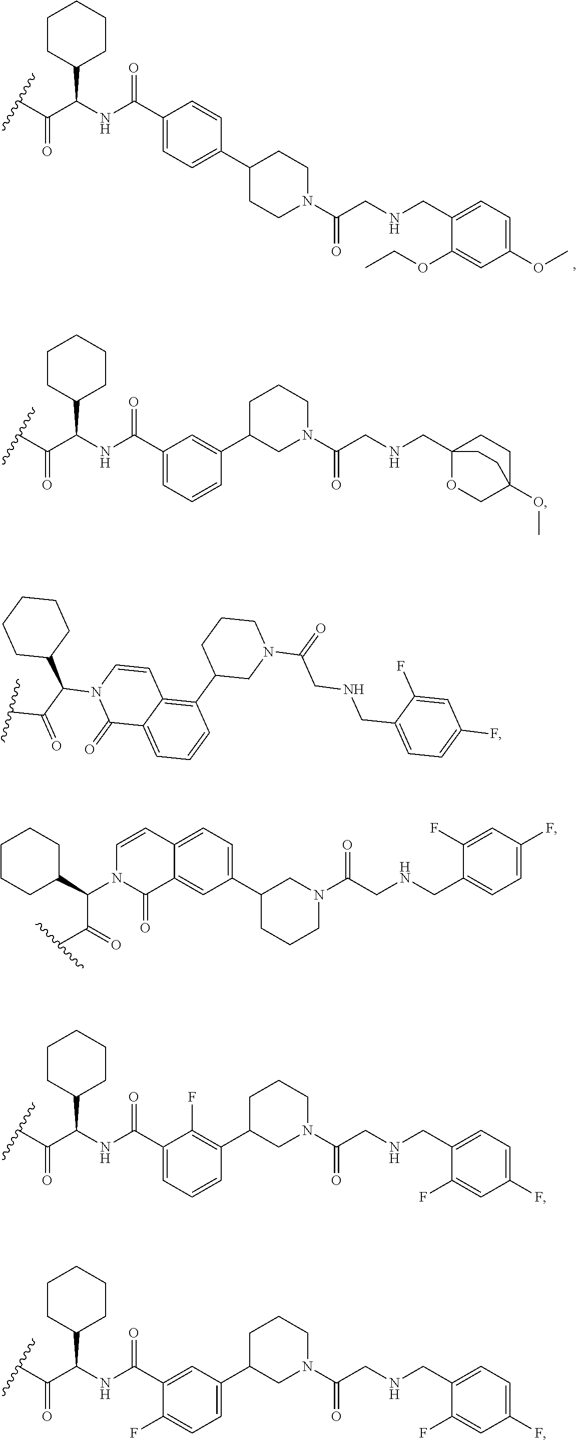 [SMILES: *C(=O)[C@H](NC(=O)c1ccc(C2CCN(C(=O)CNCc3ccc(OC)cc3OCC)CC2)cc1)C1CCCCC1 ; *C(=O)[C@H](NC(=O)c1cccc(C2CCCN(C(=O)CNCC34CCC(OC)(CC3)CO4)C2)c1)C1CCCCC1 ; *C(=O)[C@@H](C1CCCCC1)n1ccc2c(C3CCCN(C(=O)CNCc4ccc(F)cc4F)C3)cccc2c1=O ; *C(=O)[C@@H](C1CCCCC1)n1ccc2ccc(C3CCCN(C(=O)CNCc4ccc(F)cc4F)C3)cc2c1=O ; *C(=O)[C@H](NC(=O)c1cccc(C2CCCN(C(=O)CNCc3ccc(F)cc3F)C2)c1F)C1CCCCC1 ; *C(=O)[C@H](NC(=O)c1cc(C2CCCN(C(=O)CNCc3ccc(F)cc3F)C2)ccc1F)C1CCCCC1]

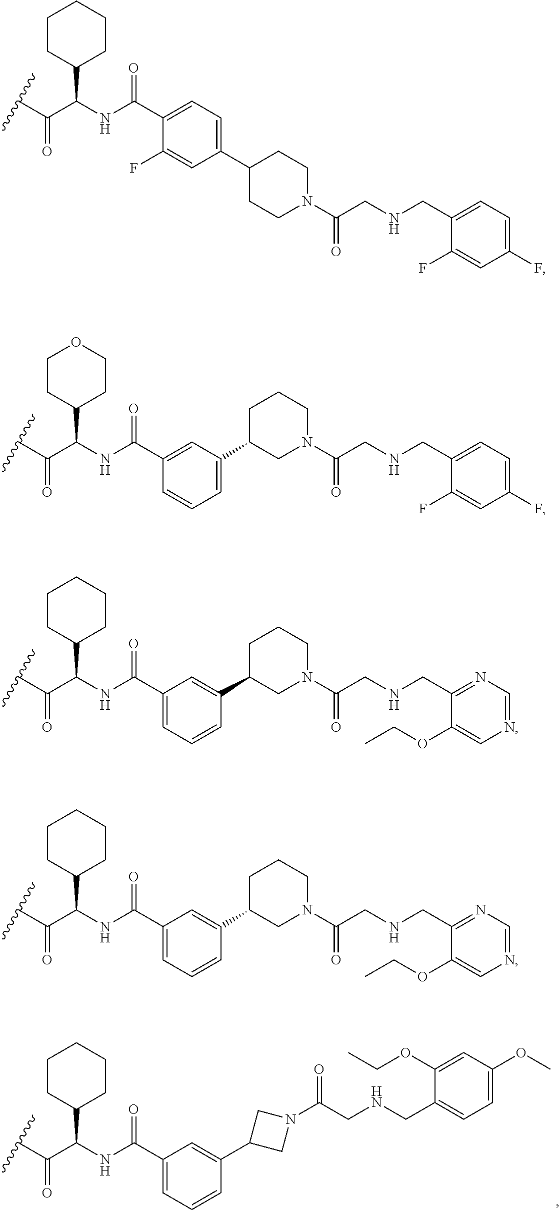 [SMILES: *C(=O)[C@H](NC(=O)c1ccc(C2CCN(C(=O)CNCc3ccc(F)cc3F)CC2)cc1F)C1CCCCC1 ; *C(=O)[C@H](NC(=O)c1cccc([C@@H]2CCCN(C(=O)CNCc3ccc(F)cc3F)C2)c1)C1CCOCC1 ; *C(=O)[C@H](NC(=O)c1cccc([C@H]2CCCN(C(=O)CNCc3ncncc3OCC)C2)c1)C1CCCCC1 ; *C(=O)[C@H](NC(=O)c1cccc([C@@H]2CCCN(C(=O)CNCc3ncncc3OCC)C2)c1)C1CCCCC1 ; *C(=O)[C@H](NC(=O)c1cccc(C2CN(C(=O)CNCc3ccc(OC)cc3OCC)C2)c1)C1CCCCC1]

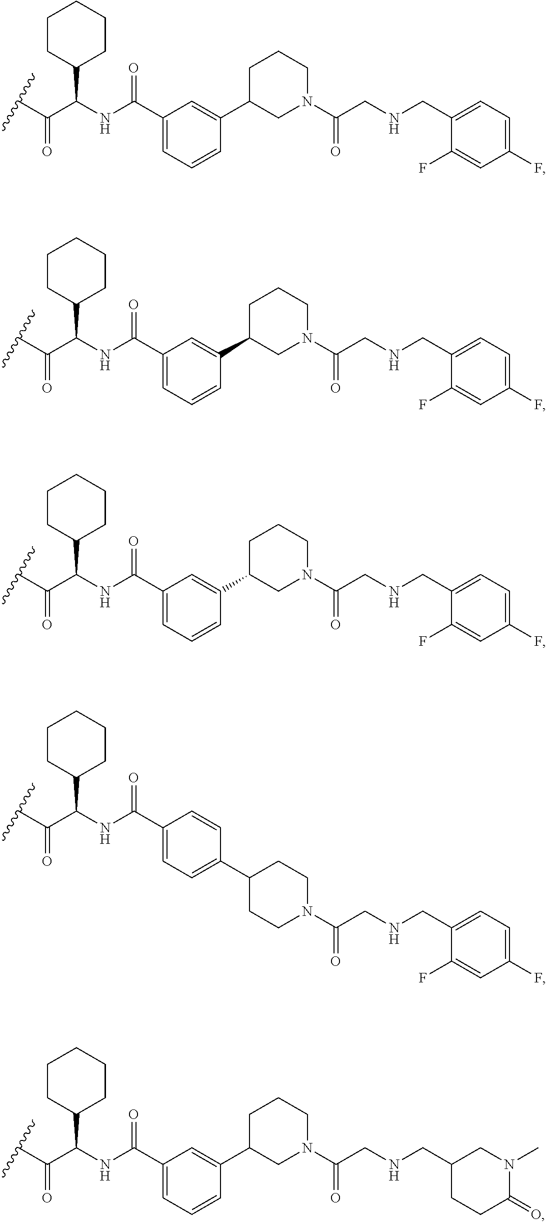 [SMILES: *C(=O)[C@H](NC(=O)c1cccc(C2CCCN(C(=O)CNCc3ccc(F)cc3F)C2)c1)C1CCCCC1 ; *C(=O)[C@H](NC(=O)c1cccc([C@H]2CCCN(C(=O)CNCc3ccc(F)cc3F)C2)c1)C1CCCCC1 ; *C(=O)[C@H](NC(=O)c1cccc([C@@H]2CCCN(C(=O)CNCc3ccc(F)cc3F)C2)c1)C1CCCCC1 ; *C(=O)[C@H](NC(=O)c1ccc(C2CCN(C(=O)CNCc3ccc(F)cc3F)CC2)cc1)C1CCCCC1 ; *C(=O)[C@H](NC(=O)c1cccc(C2CCCN(C(=O)CNCC3CCC(=O)N(C)C3)C2)c1)C1CCCCC1]

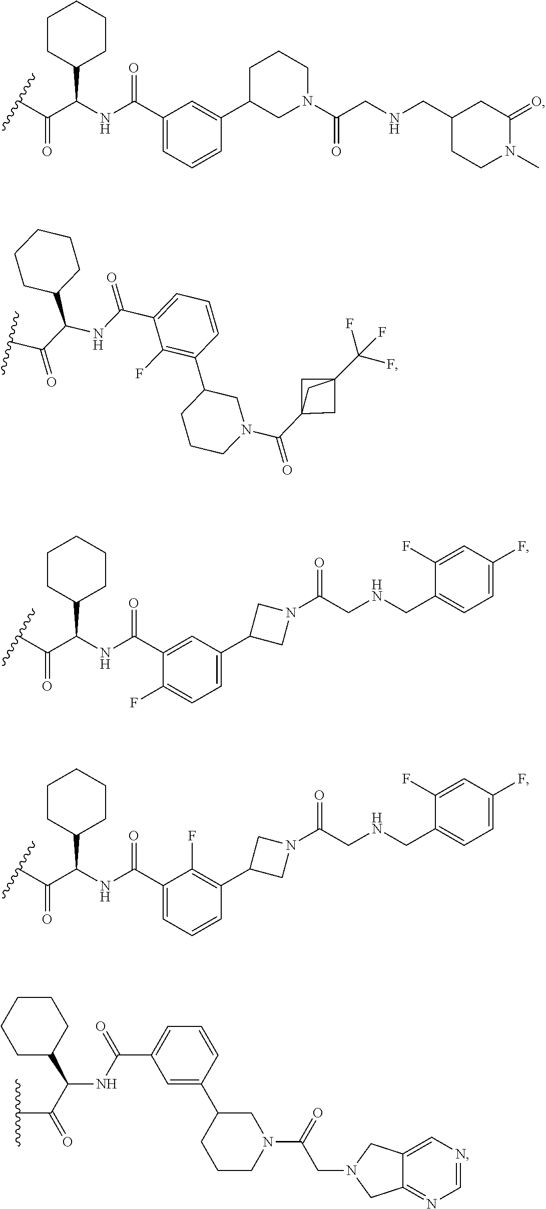 [SMILES: *C(=O)[C@H](NC(=O)c1cccc(C2CCCN(C(=O)CNCC3CCN(C)C(=O)C3)C2)c1)C1CCCCC1 ; *C(=O)[C@H](NC(=O)c1cccc(C2CCCN(C(=O)C34CC(C(F)(F)F)(C3)C4)C2)c1F)C1CCCCC1 ; *C(=O)[C@H](NC(=O)c1cc(C2CN(C(=O)CNCc3ccc(F)cc3F)C2)ccc1F)C1CCCCC1 ; *C(=O)[C@H](NC(=O)c1cccc(C2CN(C(=O)CNCc3ccc(F)cc3F)C2)c1F)C1CCCCC1 ; *C(=O)[C@H](NC(=O)c1cccc(C2CCCN(C(=O)CN3Cc4cncnc4C3)C2)c1)C1CCCCC1]

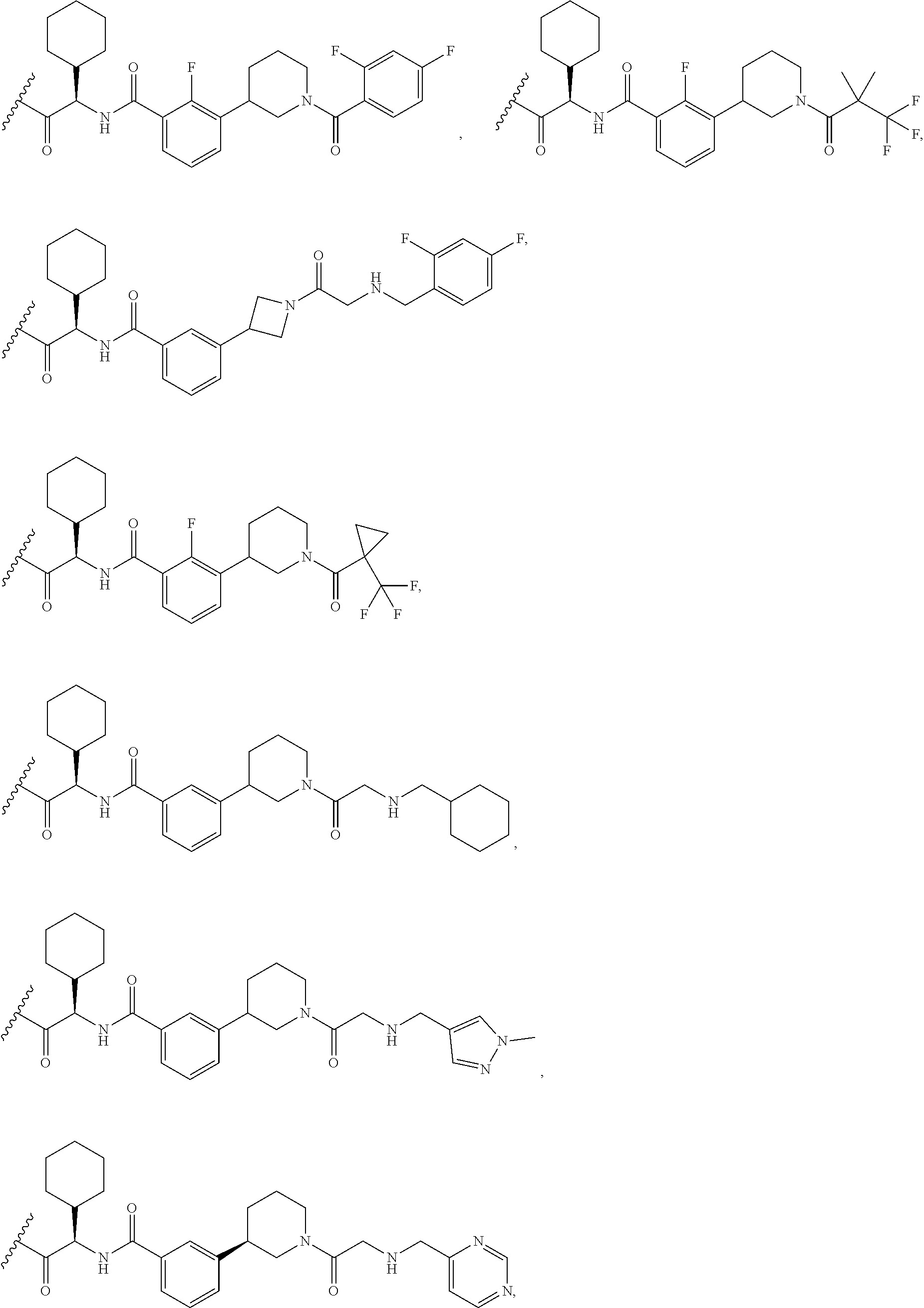

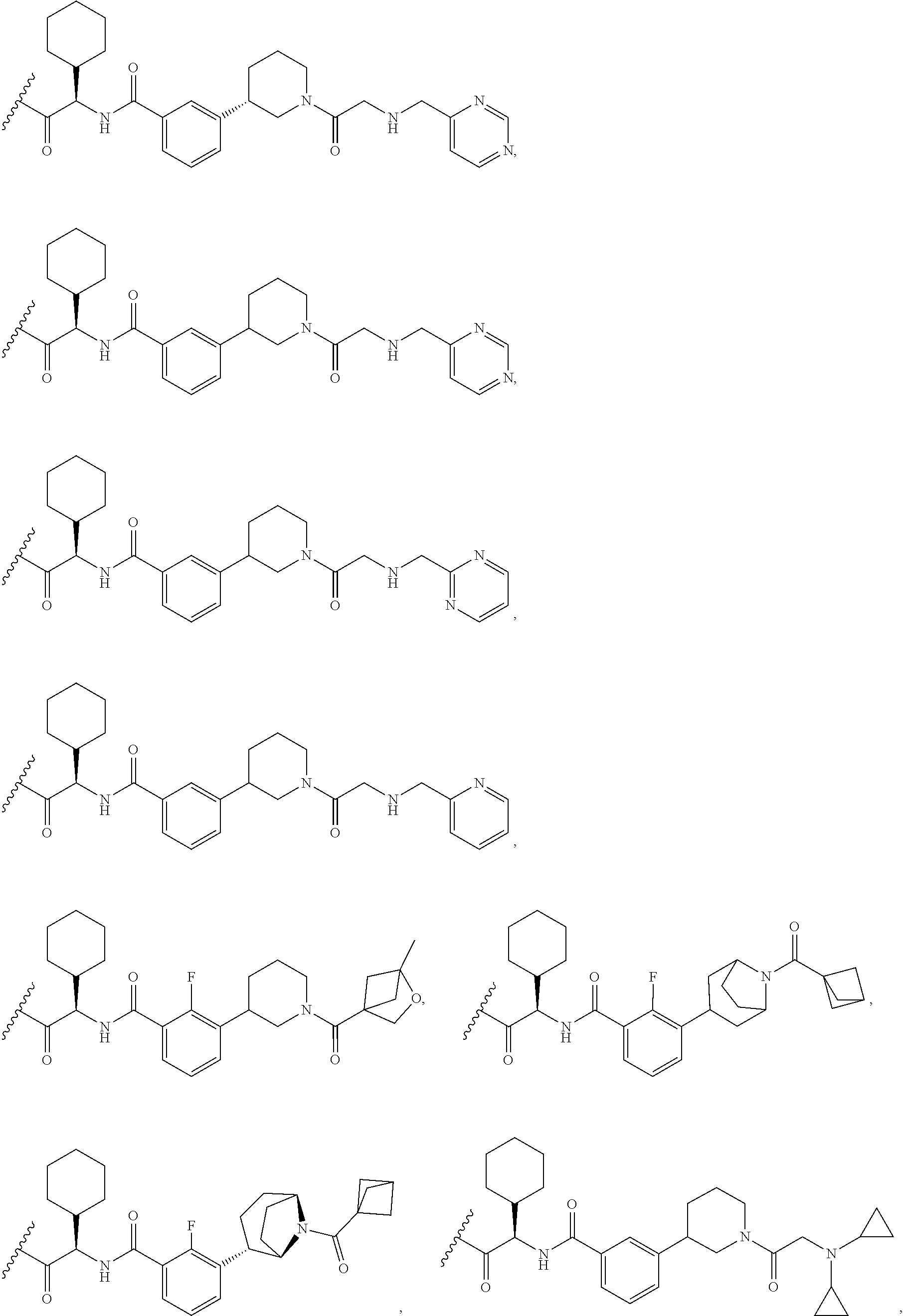

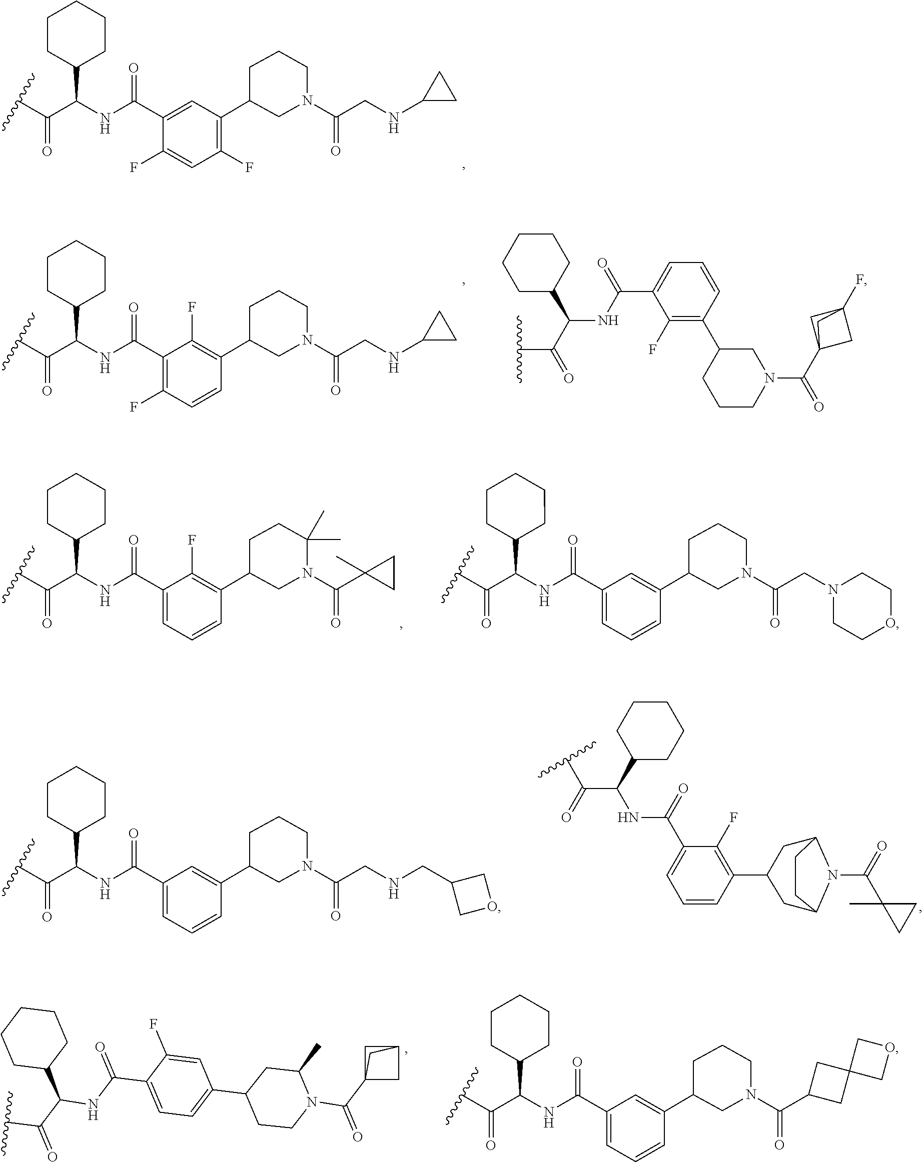

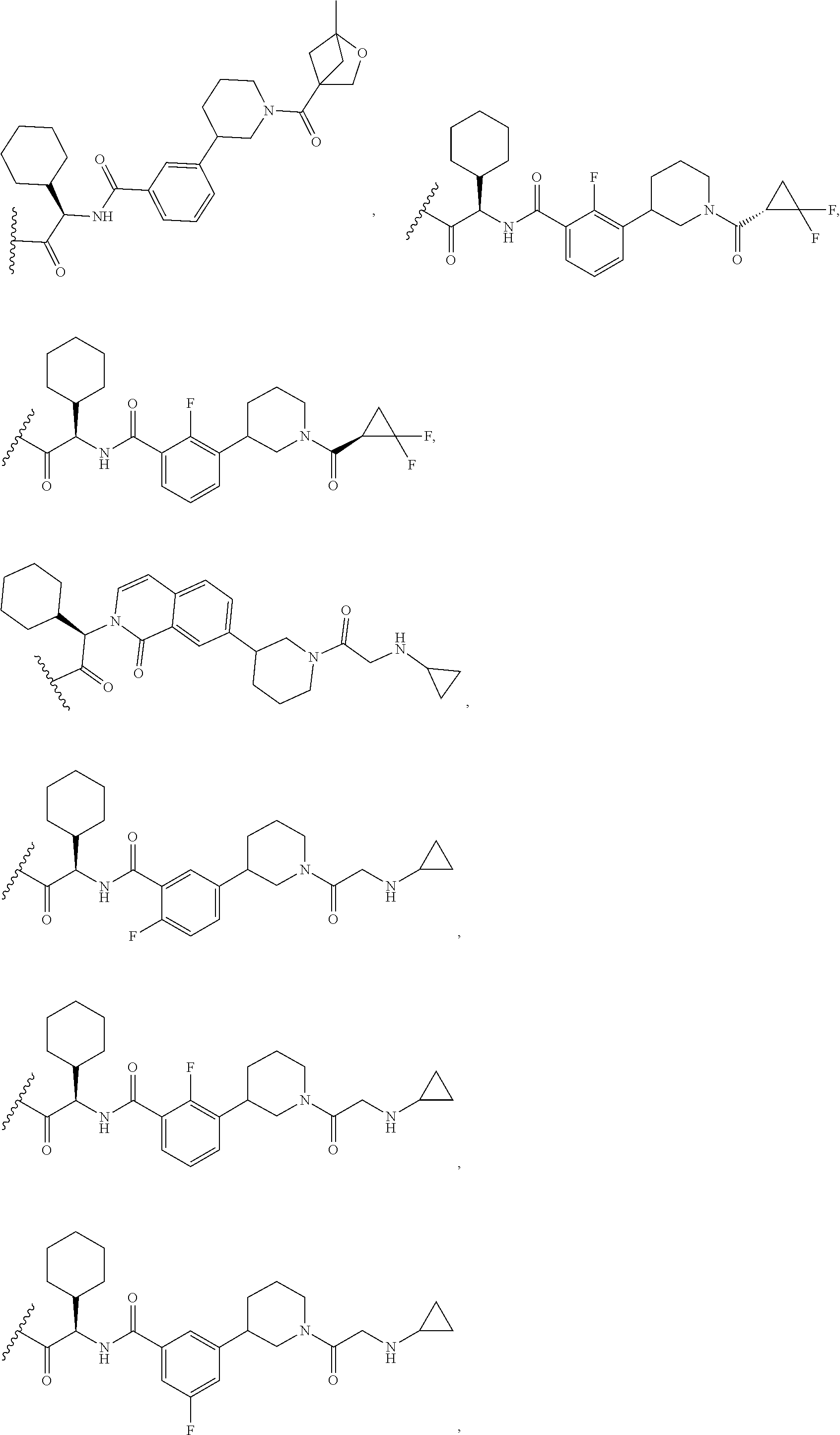

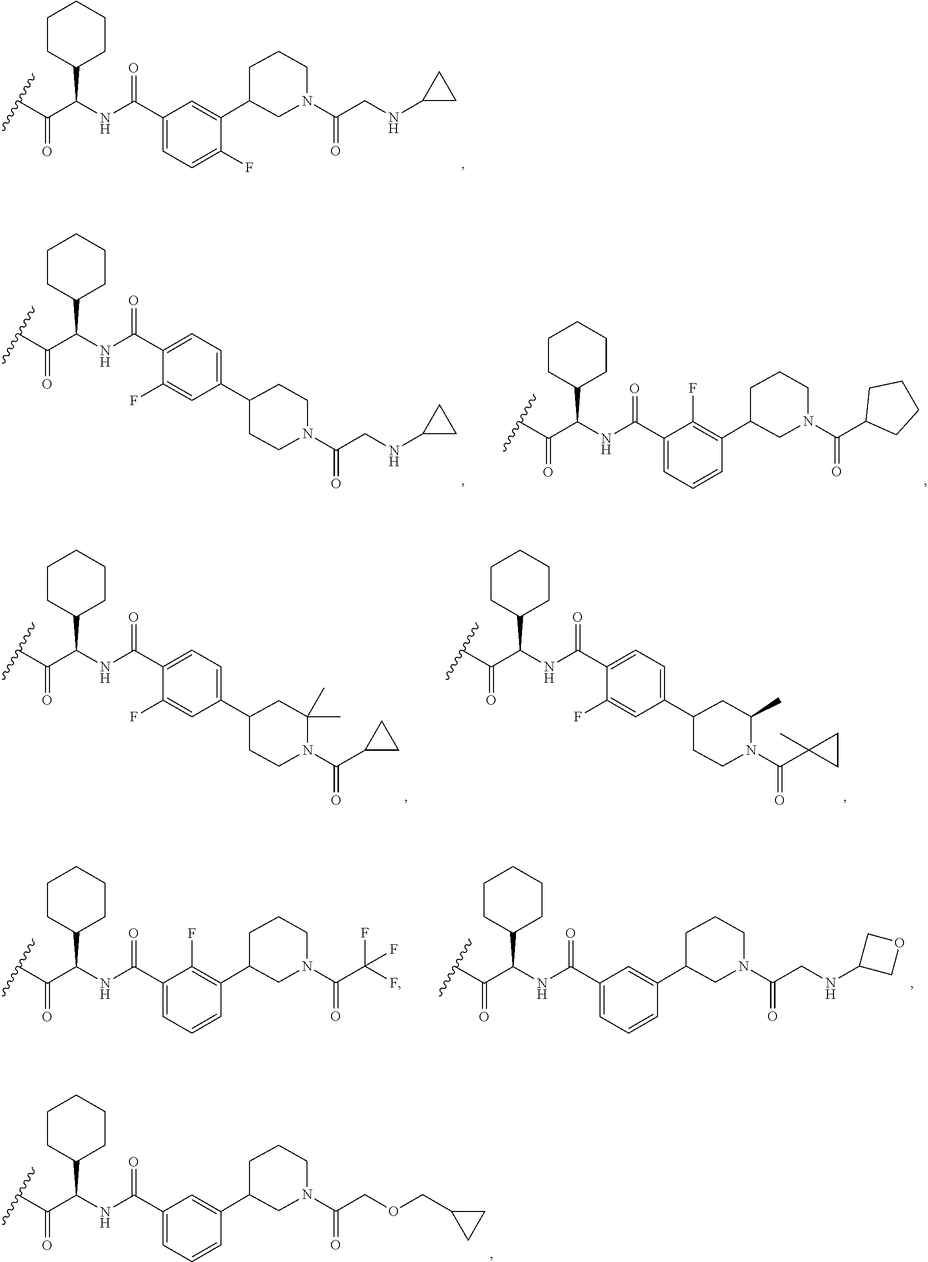

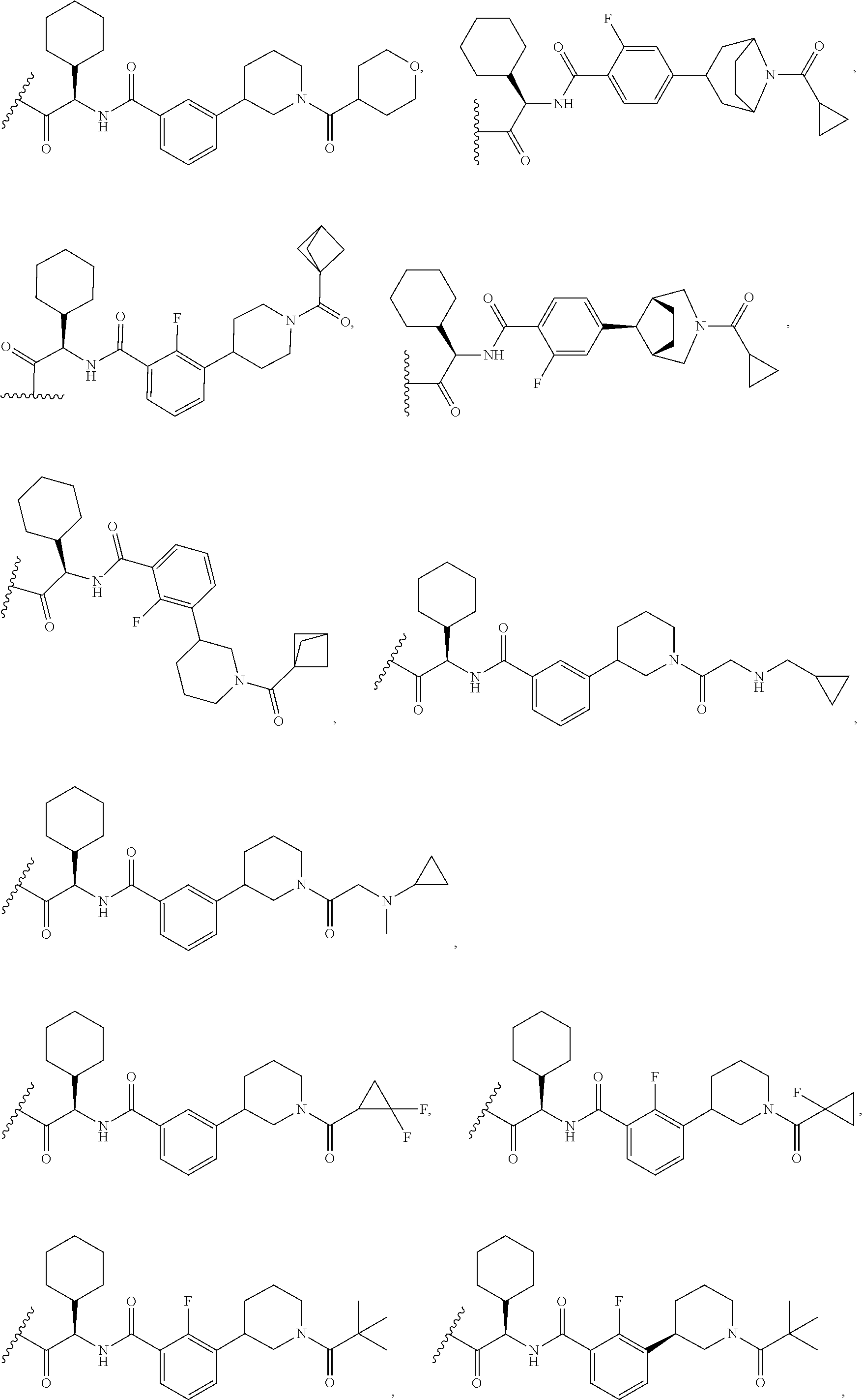

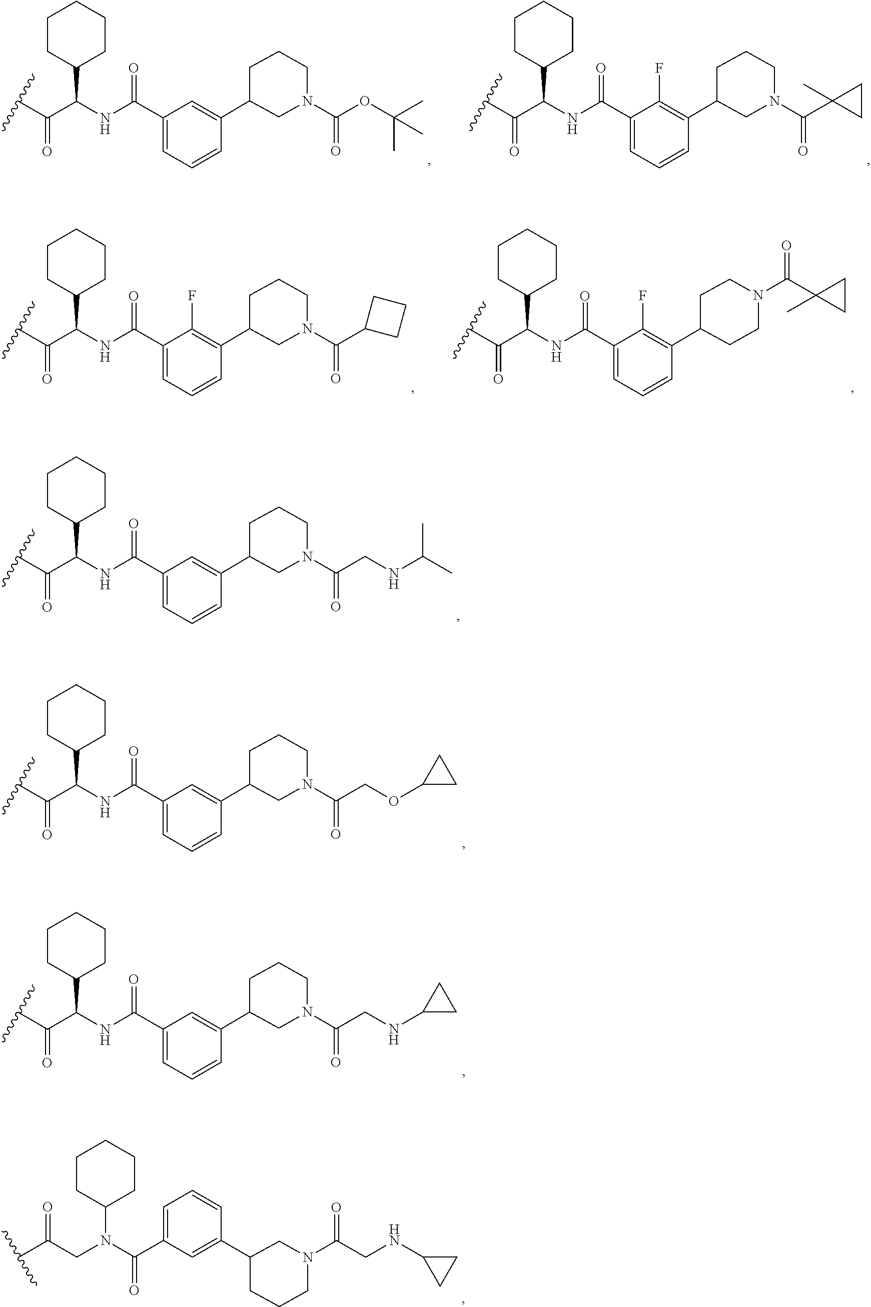

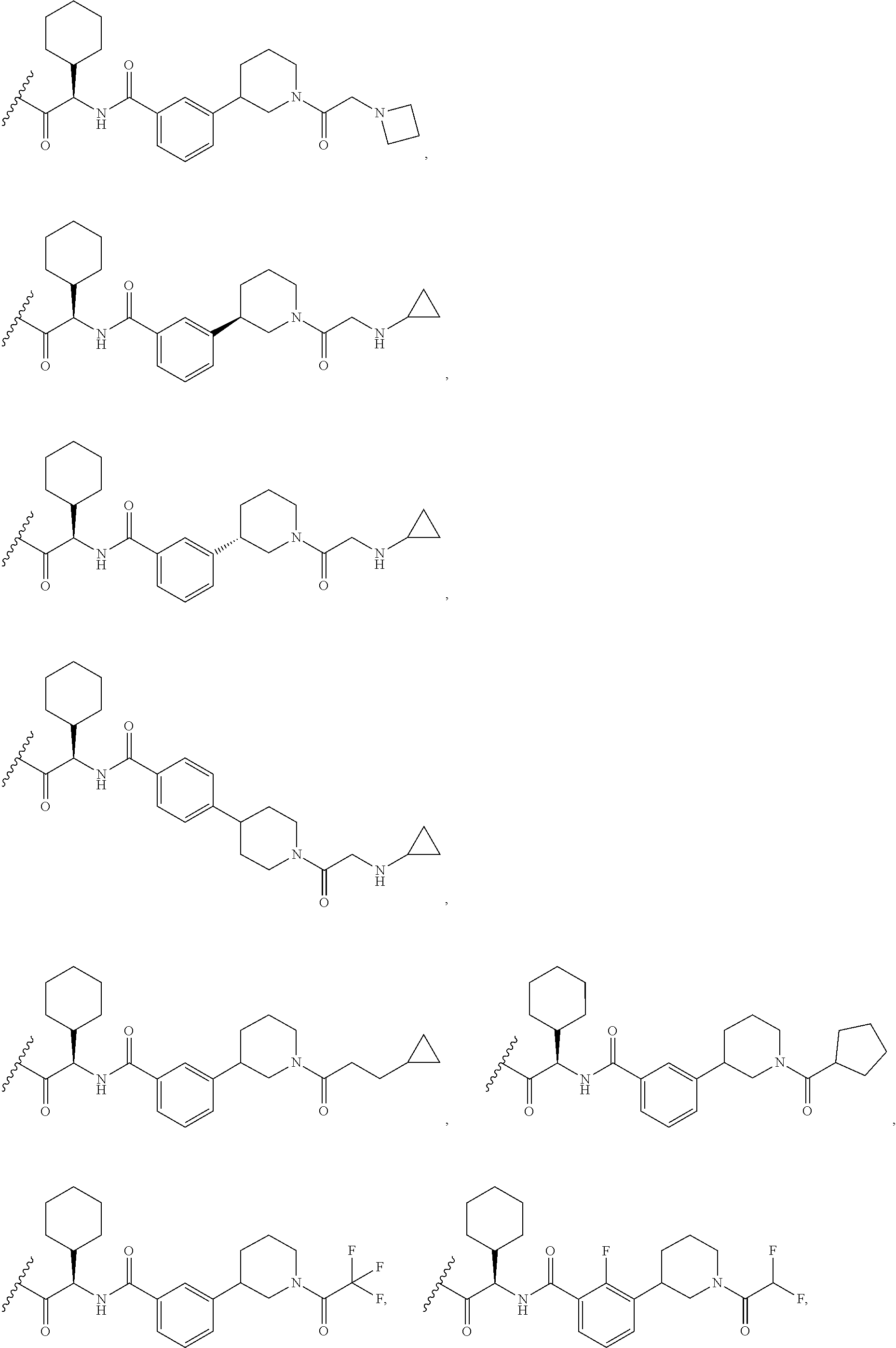

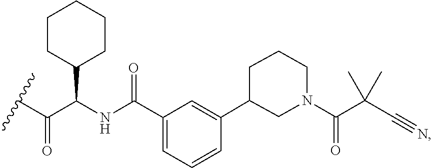 [SMILES: *C(=O)[C@H](NC(=O)c1cccc(C2CCCN(C(=O)C(C)(C)C#N)C2)c1)C1CCCCC1]

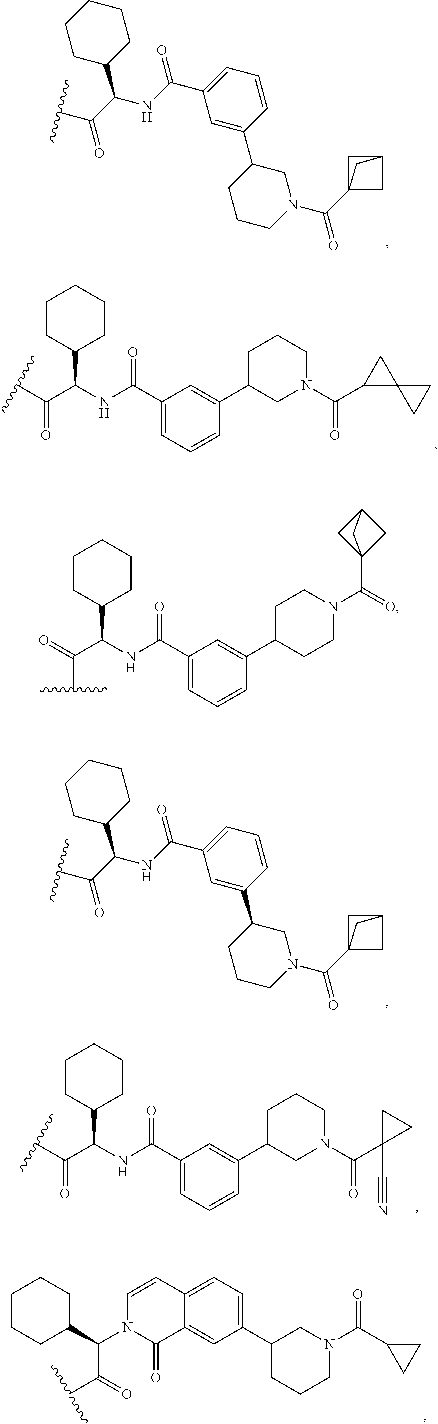 [SMILES: *C(=O)[C@H](NC(=O)c1cccc(C2CCCN(C(=O)C34CC(C3)C4)C2)c1)C1CCCCC1 ; *C(=O)[C@H](NC(=O)c1cccc(C2CCCN(C(=O)C3CC34CC4)C2)c1)C1CCCCC1 ; *C(=O)[C@H](NC(=O)c1cccc(C2CCN(C(=O)C34CC(C3)C4)CC2)c1)C1CCCCC1 ; *C(=O)[C@H](NC(=O)c1cccc([C@@H]2CCCN(C(=O)C34CC(C3)C4)C2)c1)C1CCCCC1 ; *C(=O)[C@H](NC(=O)c1cccc(C2CCCN(C(=O)C3(C#N)CC3)C2)c1)C1CCCCC1 ; *C(=O)[C@@H](C1CCCCC1)n1ccc2ccc(C3CCCN(C(=O)C4CC4)C3)cc2c1=O]

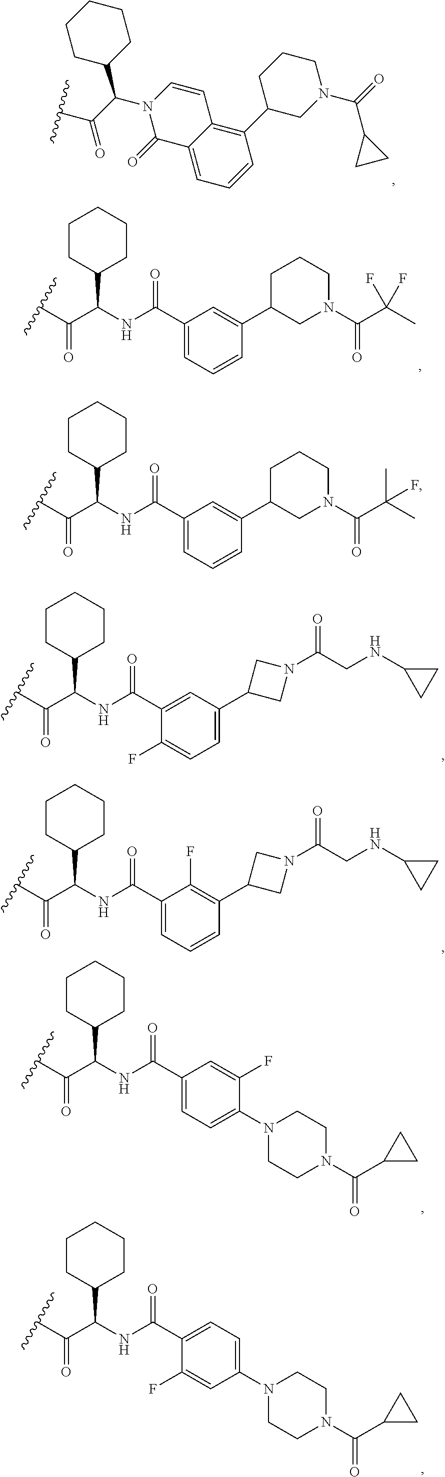

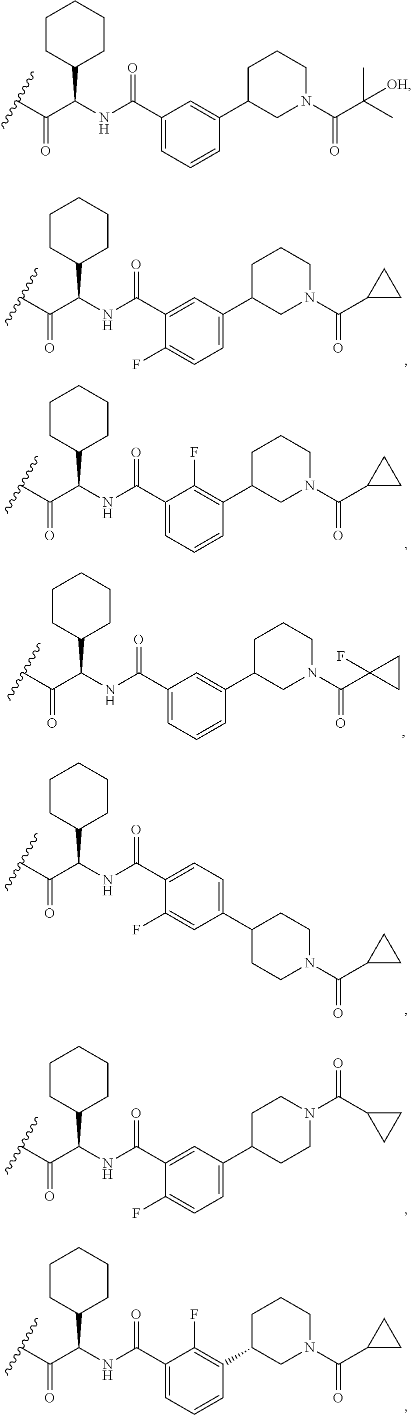

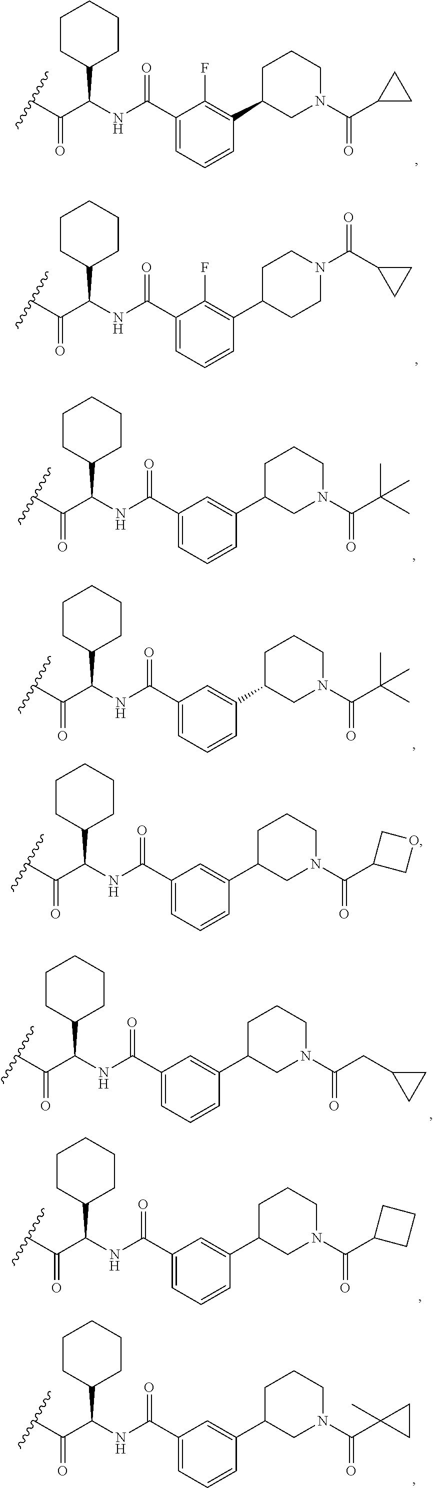

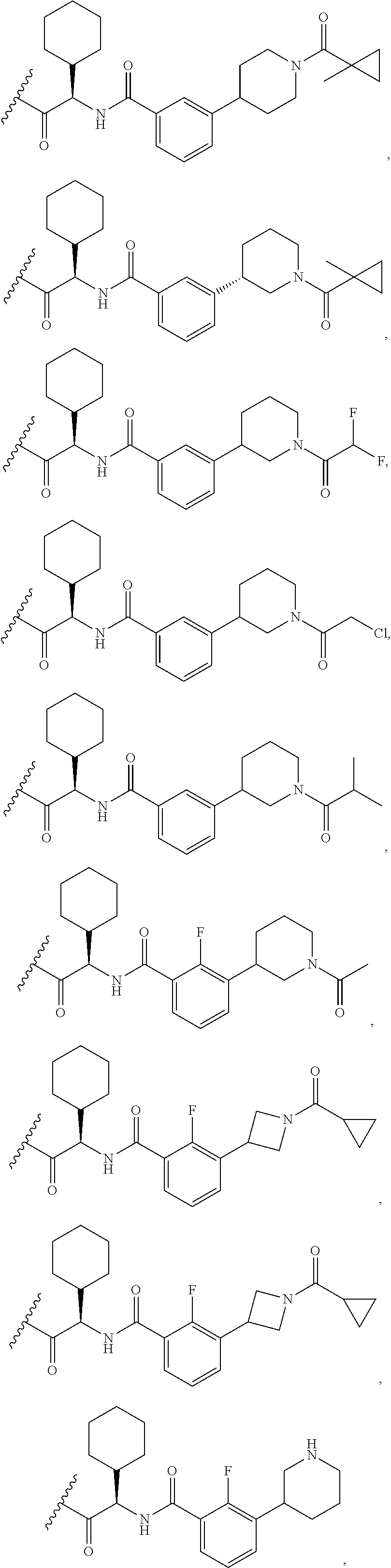

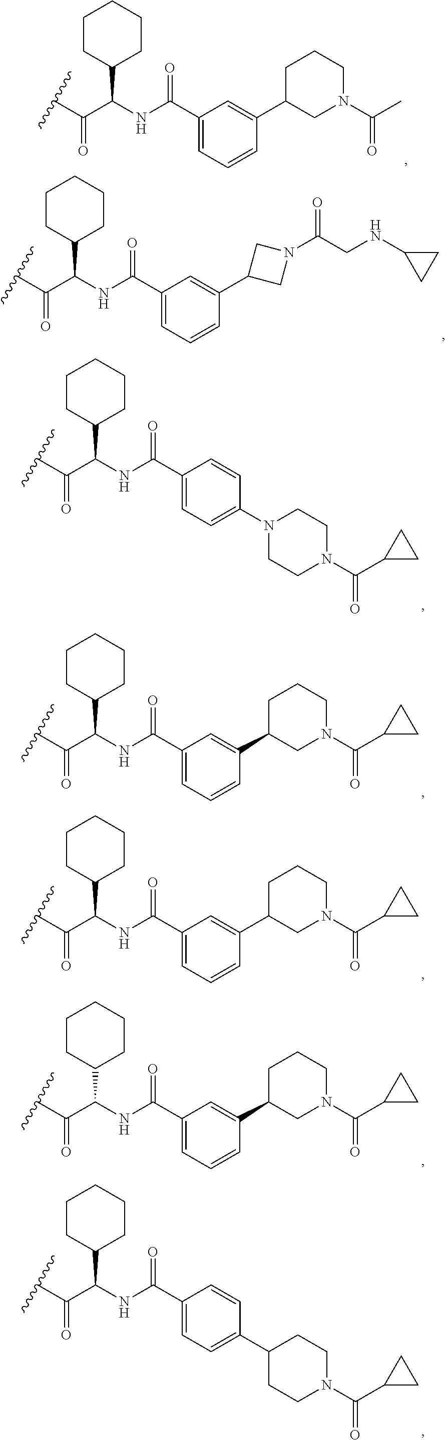

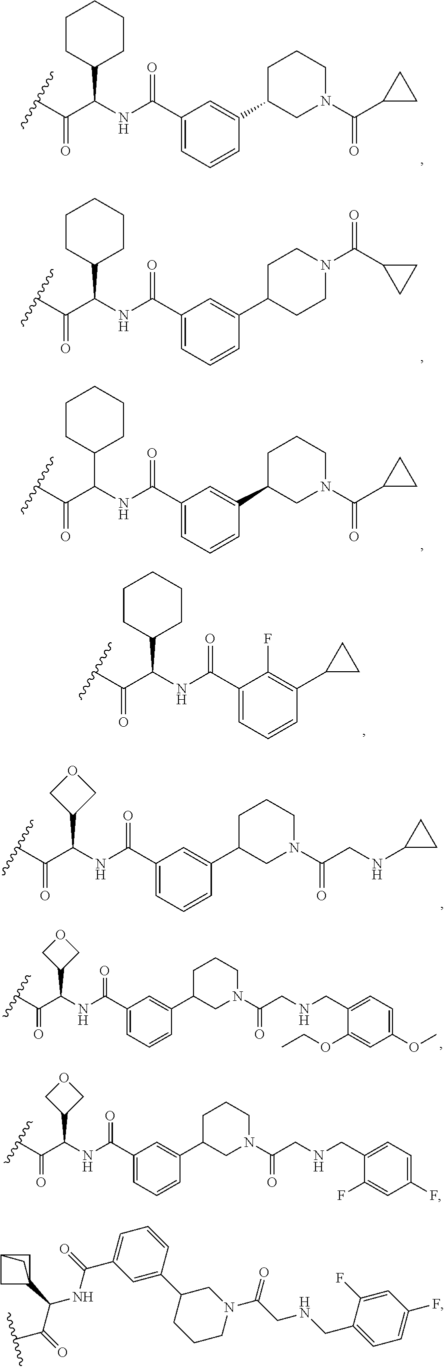

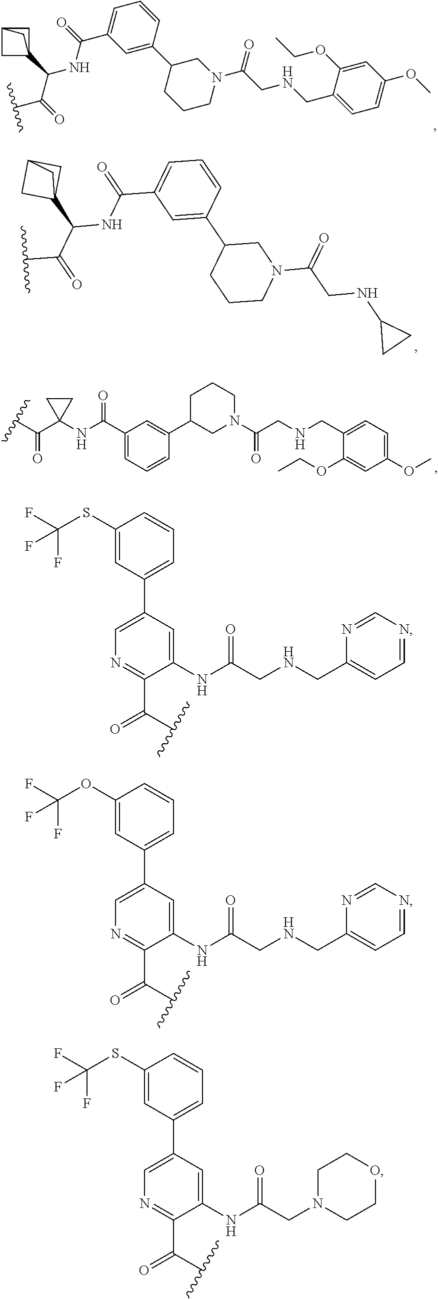

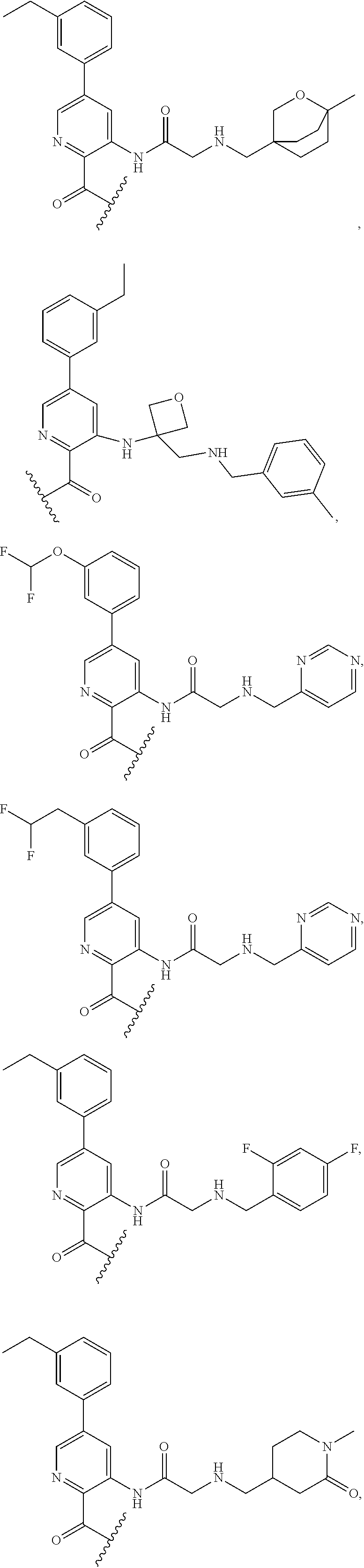

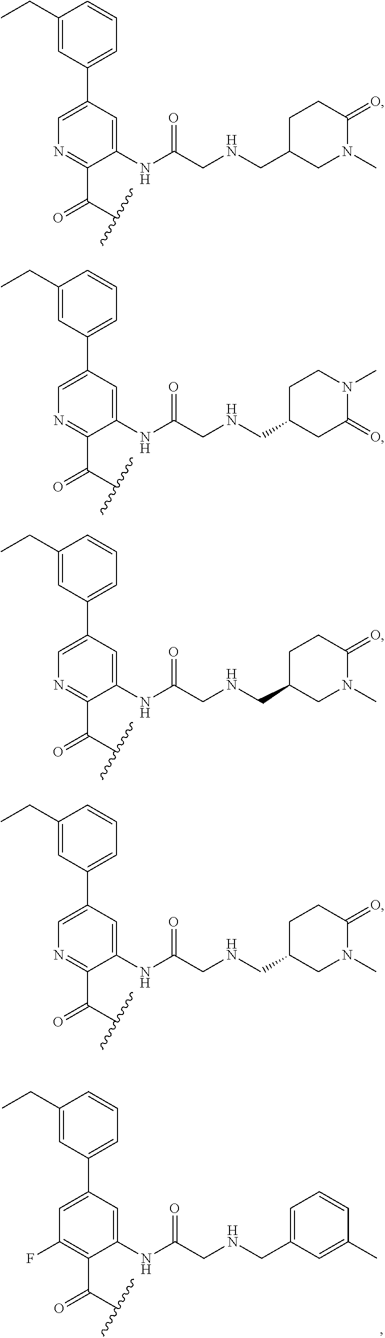

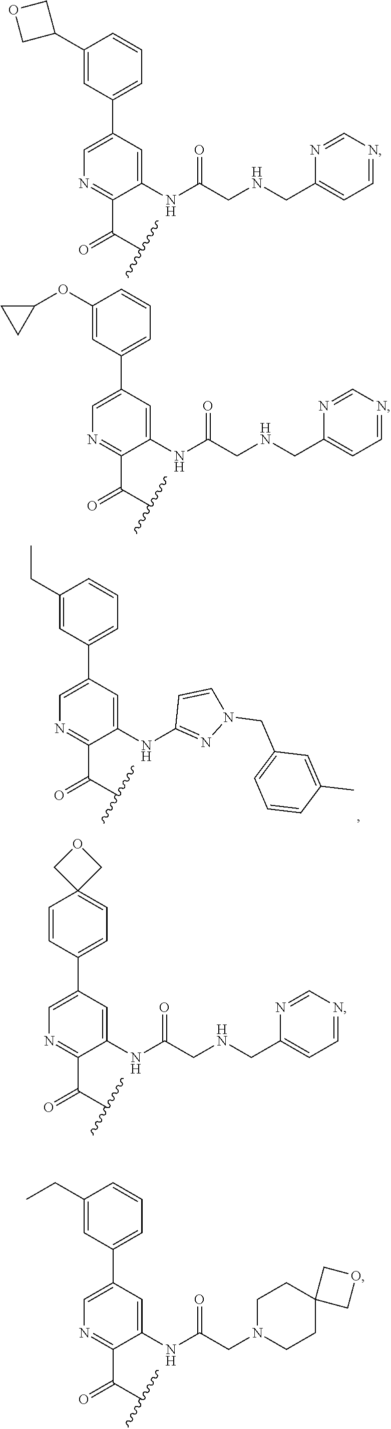

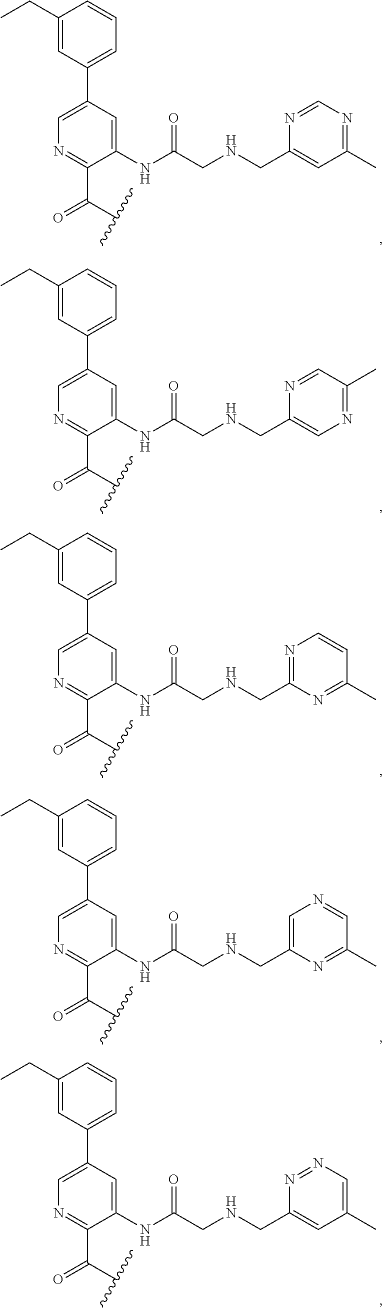

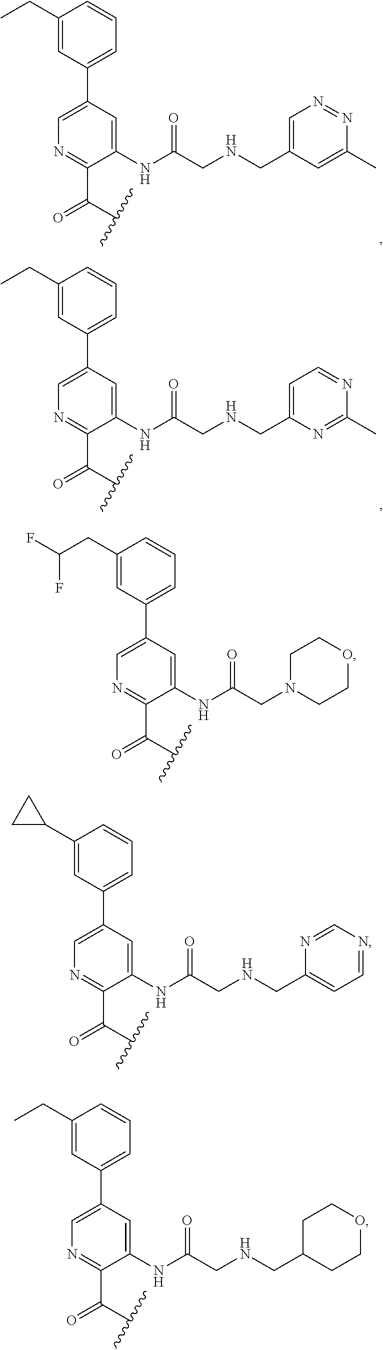

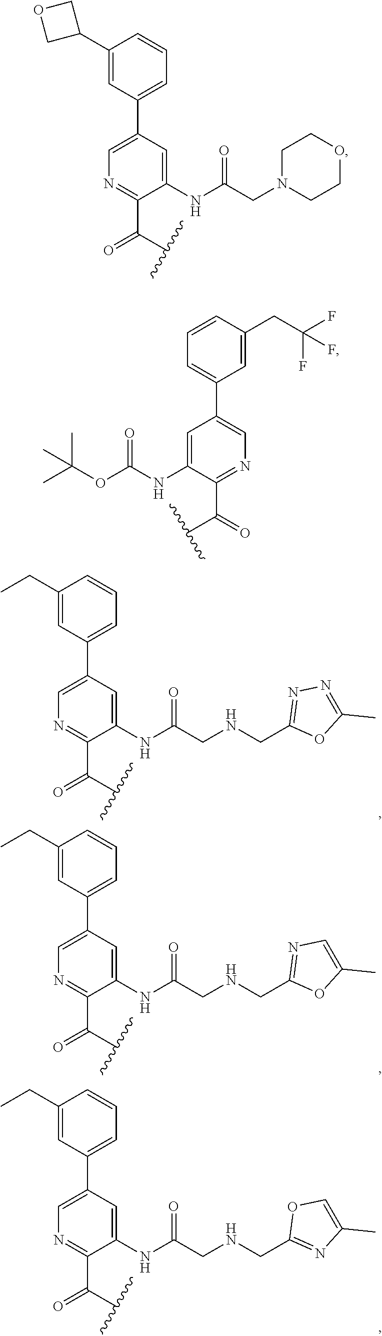

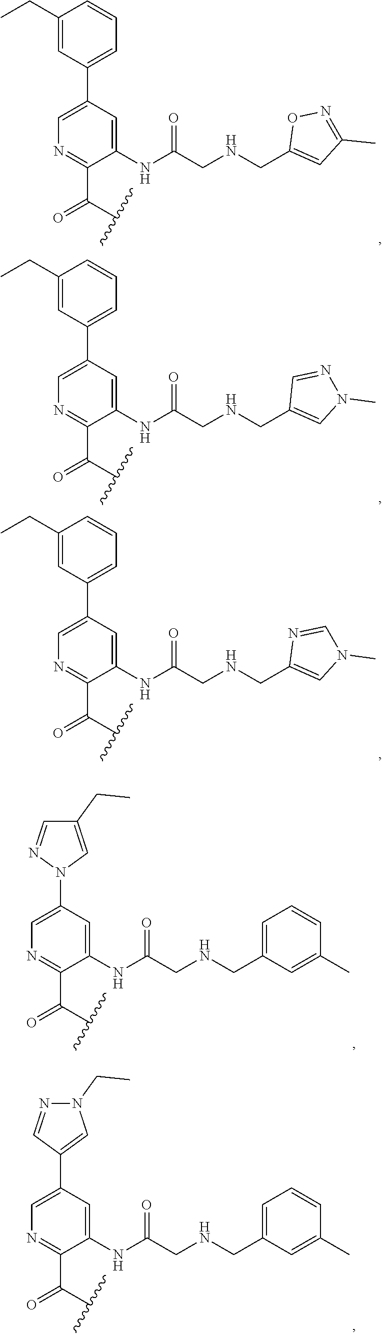

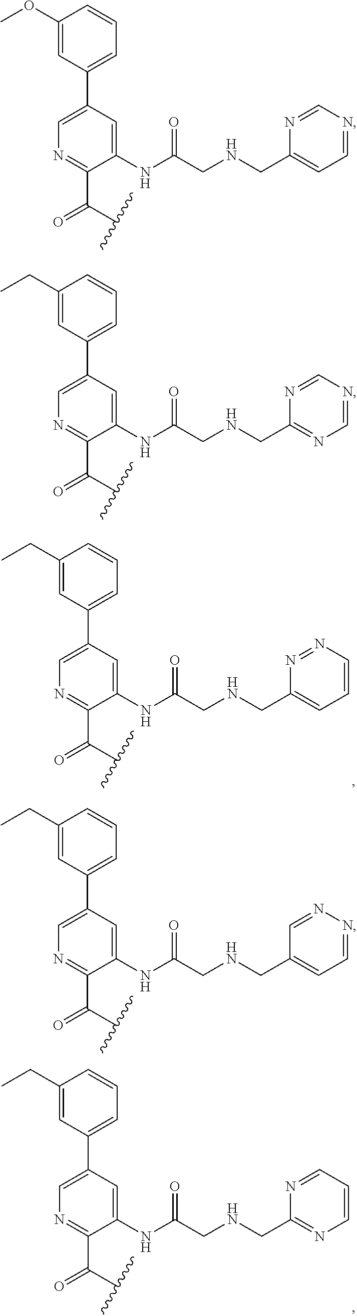 [SMILES: *C(=O)c1ncc(-c2cccc(OC)c2)cc1NC(=O)CNCc1ccncn1 ; *C(=O)c1ncc(-c2cccc(CC)c2)cc1NC(=O)CNCc1ncncn1 ; *C(=O)c1ncc(-c2cccc(CC)c2)cc1NC(=O)CNCc1cccnn1 ; *C(=O)c1ncc(-c2cccc(CC)c2)cc1NC(=O)CNCc1ccnnc1 ; *C(=O)c1ncc(-c2cccc(CC)c2)cc1NC(=O)CNCc1ncccn1]

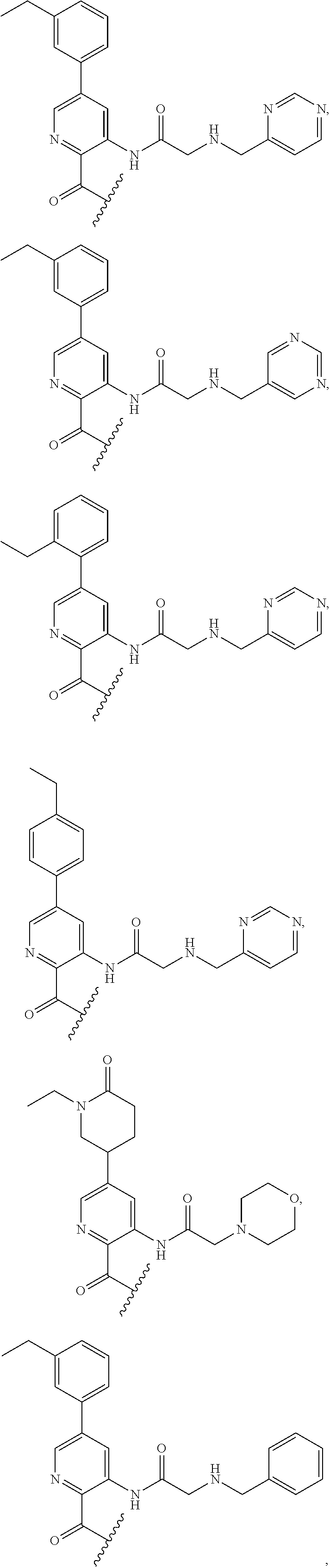 [SMILES: *C(=O)c1ncc(-c2cccc(CC)c2)cc1NC(=O)CNCc1ccncn1 ; *C(=O)c1ncc(-c2cccc(CC)c2)cc1NC(=O)CNCc1cncnc1 ; *C(=O)c1ncc(-c2ccccc2CC)cc1NC(=O)CNCc1ccncn1 ; *C(=O)c1ncc(-c2ccc(CC)cc2)cc1NC(=O)CNCc1ccncn1 ; *C(=O)c1ncc(C2CCC(=O)N(CC)C2)cc1NC(=O)CN1CCOCC1 ; *C(=O)c1ncc(-c2cccc(CC)c2)cc1NC(=O)CNCc1ccccc1]

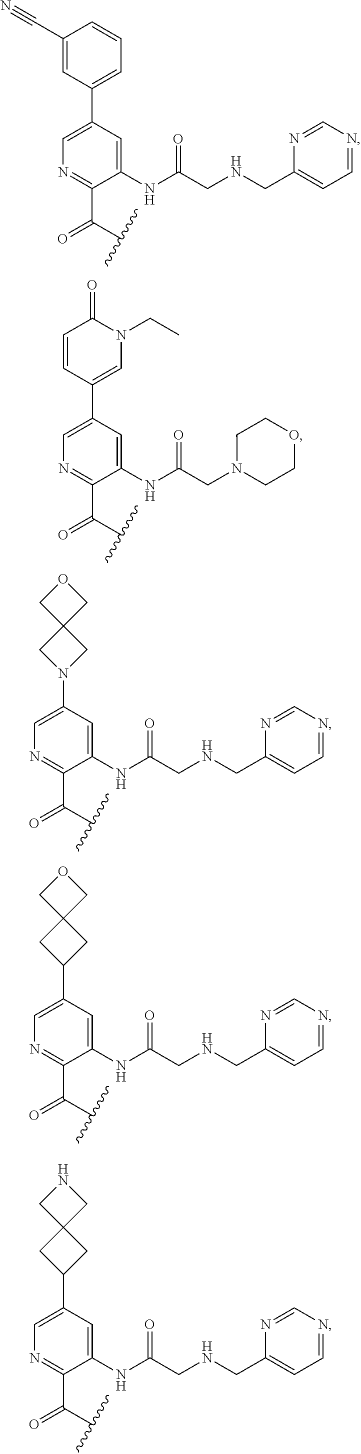

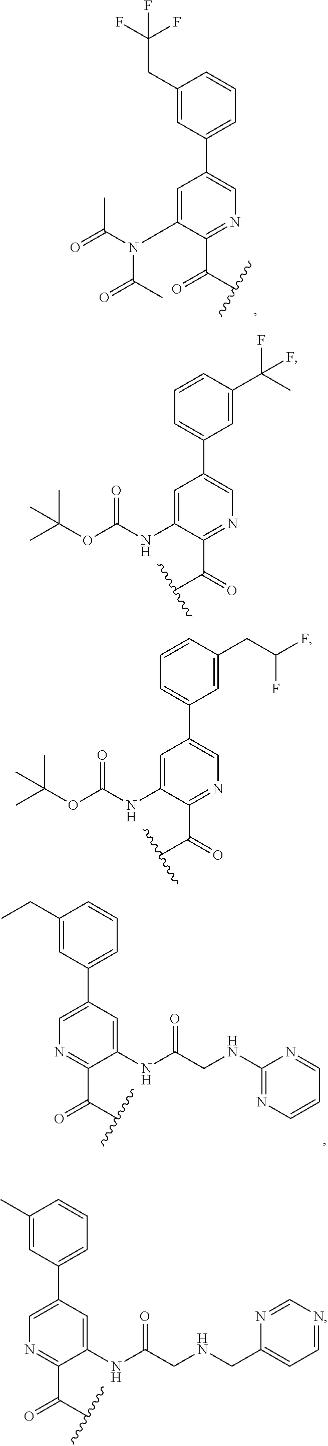 [SMILES: *C(=O)c1ncc(-c2cccc(CC(F)(F)F)c2)cc1N(C(C)=O)C(C)=O ; *C(=O)c1ncc(-c2cccc(C(C)(F)F)c2)cc1NC(=O)OC(C)(C)C ; *C(=O)c1ncc(-c2cccc(CC(F)F)c2)cc1NC(=O)OC(C)(C)C ; *C(=O)c1ncc(-c2cccc(CC)c2)cc1NC(=O)CNc1ncccn1 ; *C(=O)c1ncc(-c2cccc(C)c2)cc1NC(=O)CNCc1ccncn1]

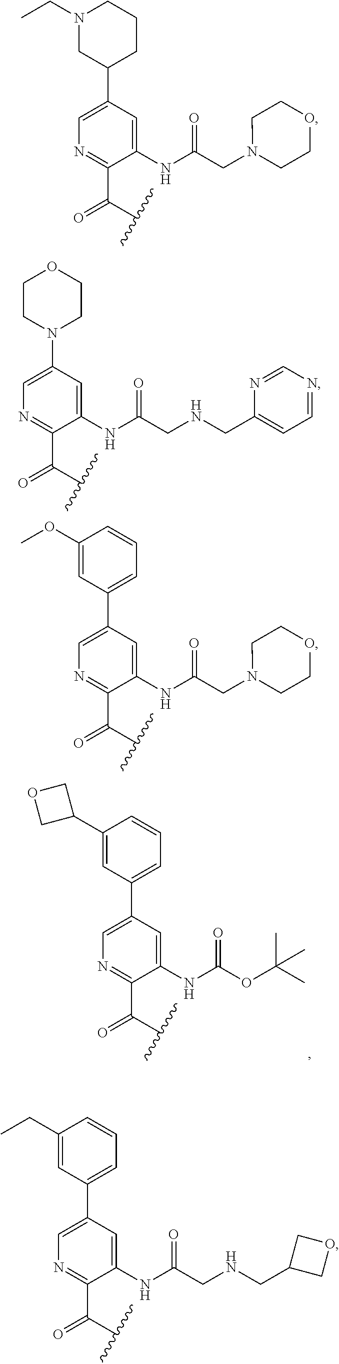

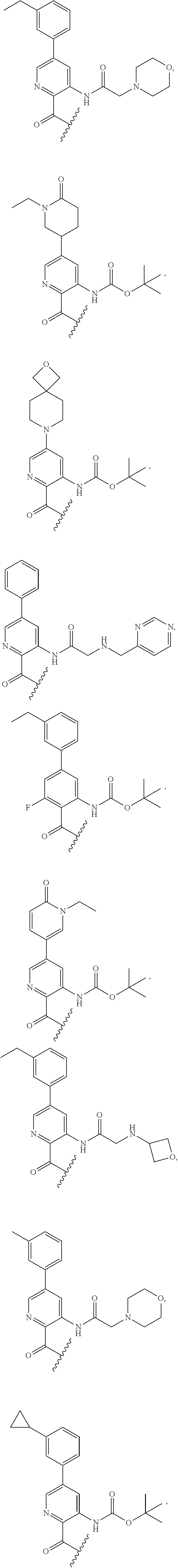

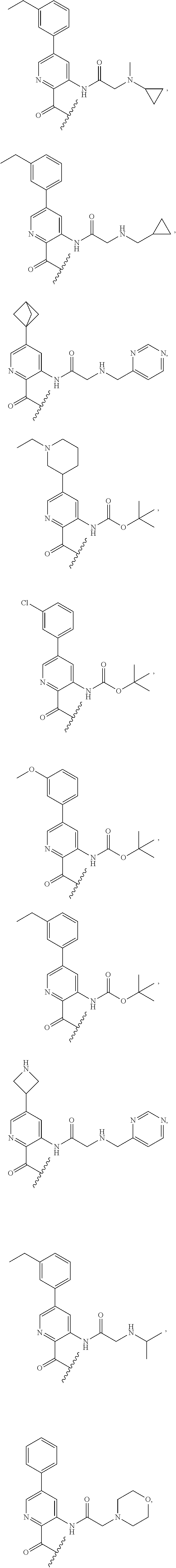

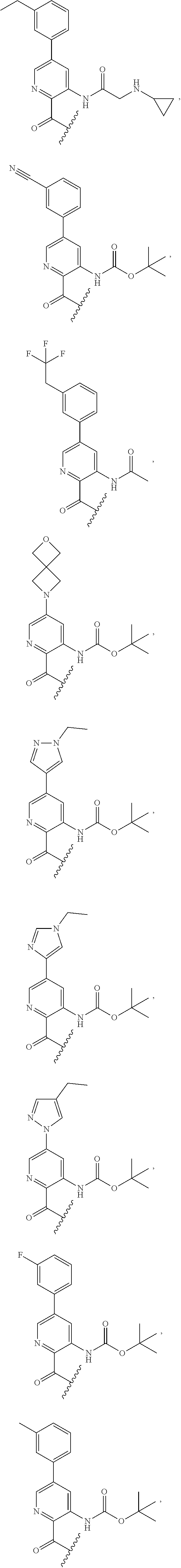

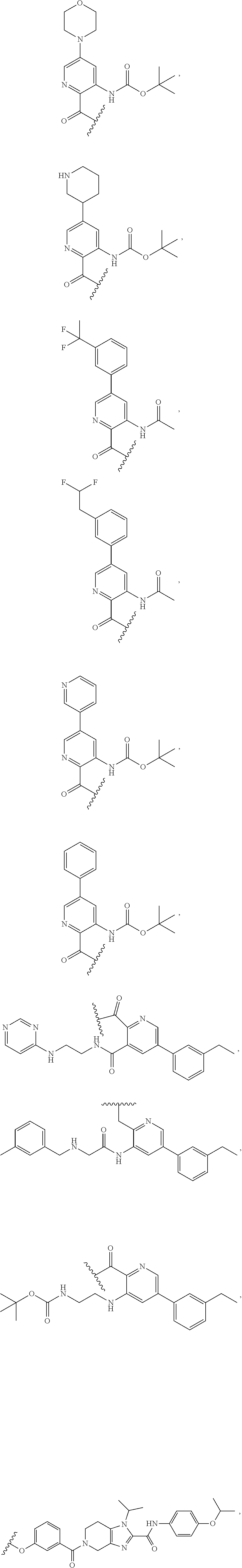

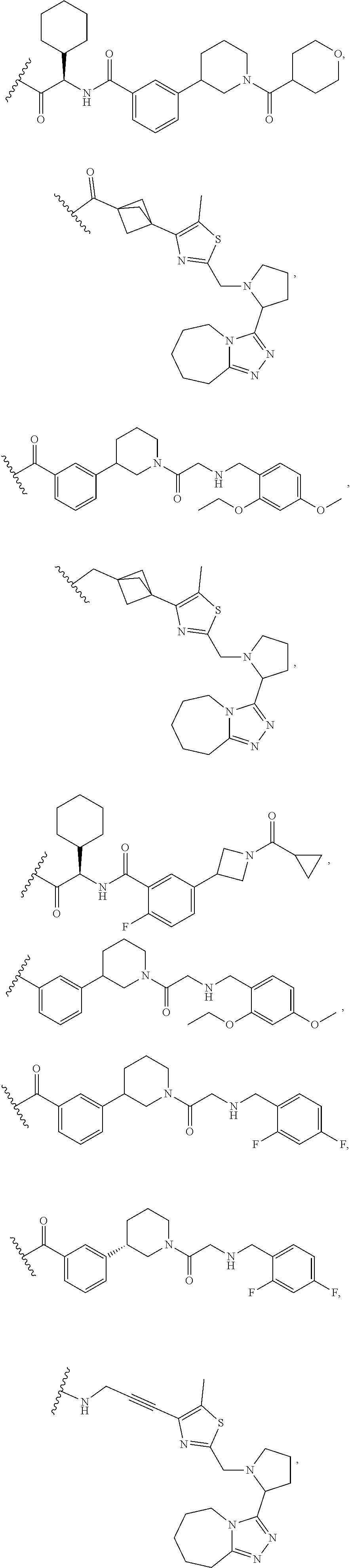

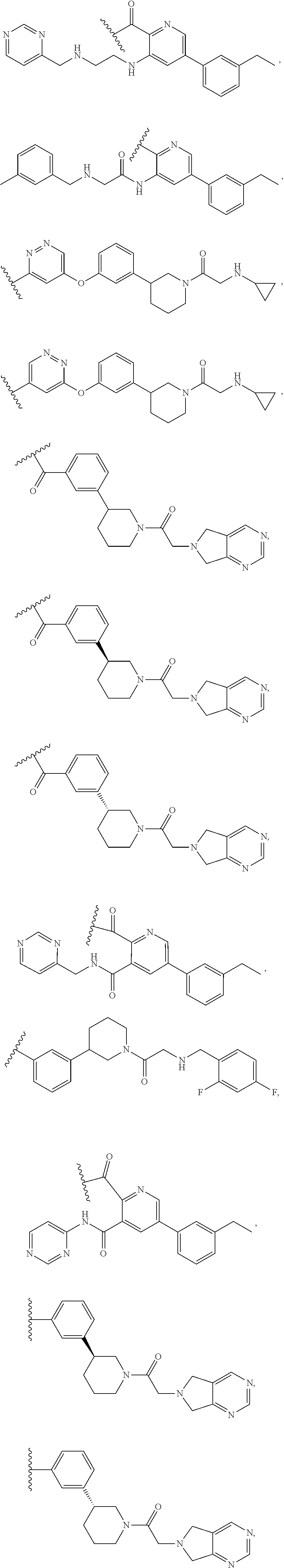

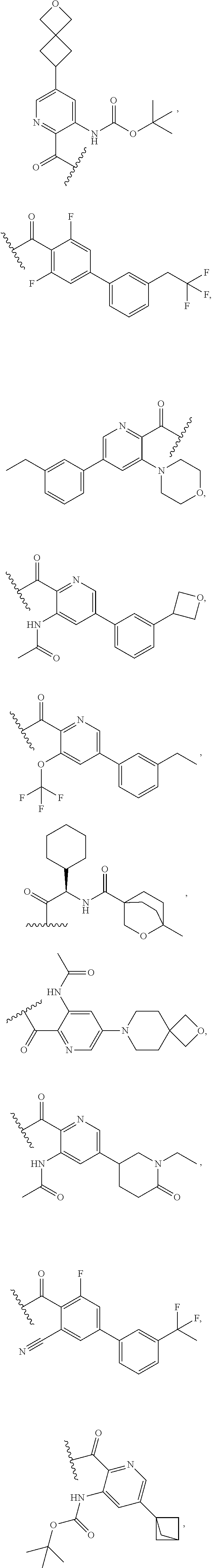

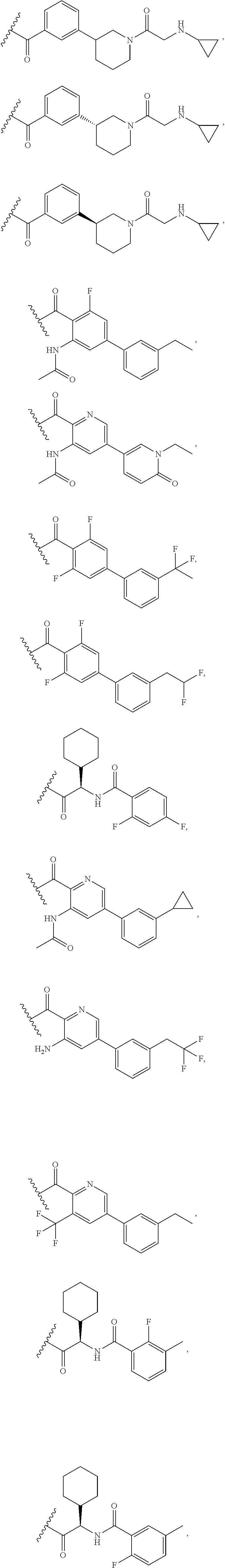

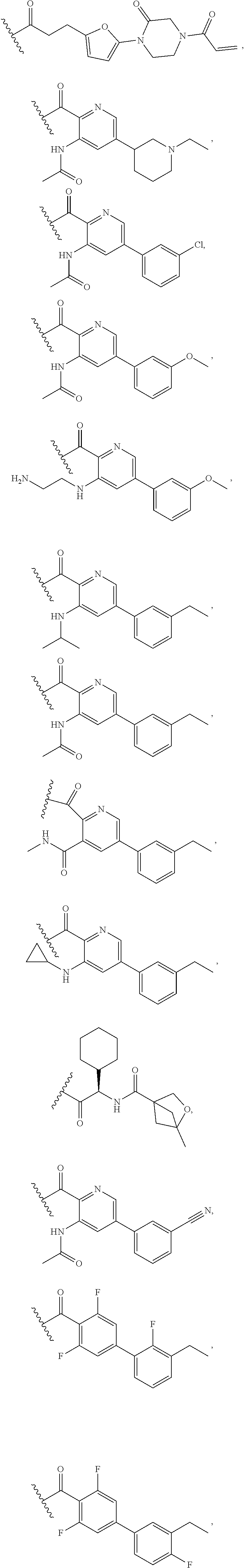

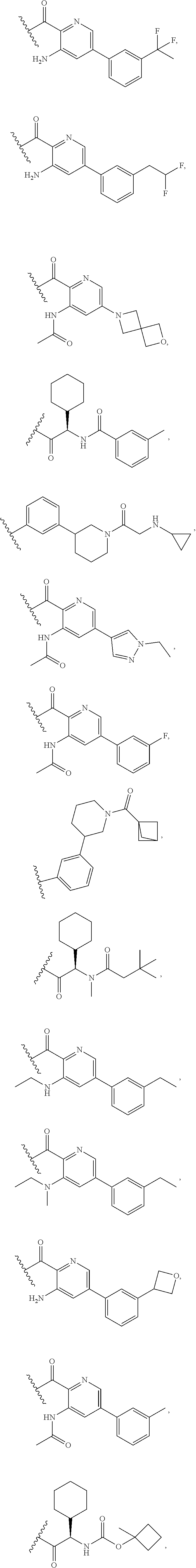

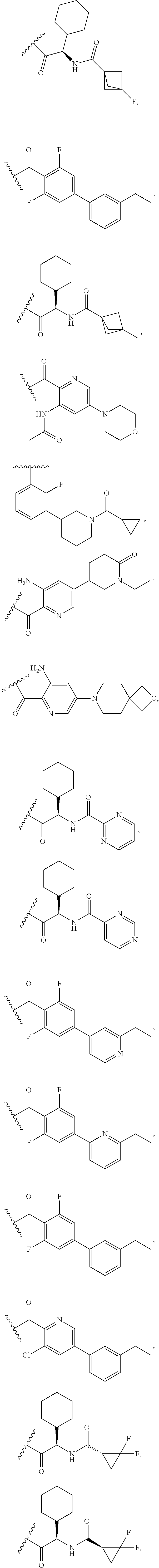

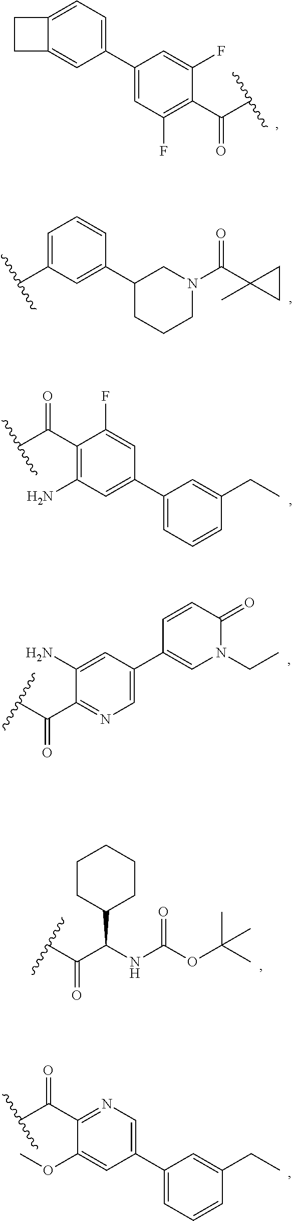 [SMILES: *C(=O)c1c(F)cc(-c2ccc3c(c2)CC3)cc1F ; *c1cccc(C2CCCN(C(=O)C3(C)CC3)C2)c1 ; *C(=O)c1c(N)cc(-c2cccc(CC)c2)cc1F ; *C(=O)c1ncc(-c2ccc(=O)n(CC)c2)cc1N ; *C(=O)[C@H](NC(=O)OC(C)(C)C)C1CCCCC1 ; *C(=O)c1ncc(-c2cccc(CC)c2)cc1OC]

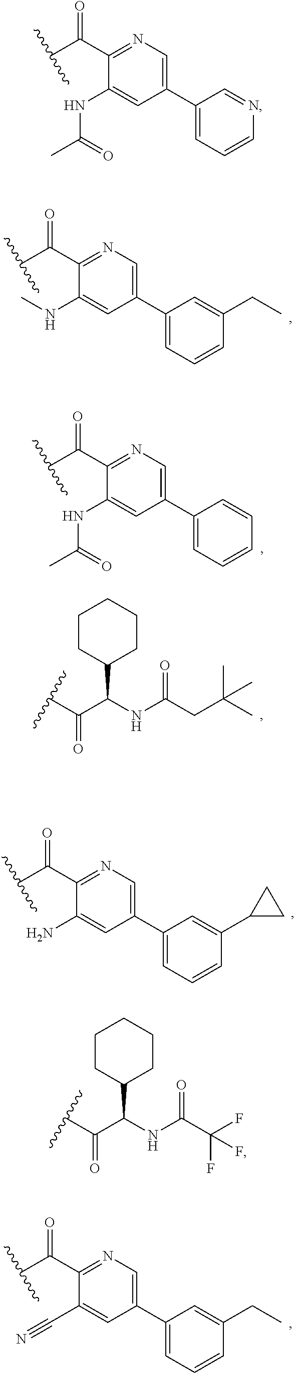

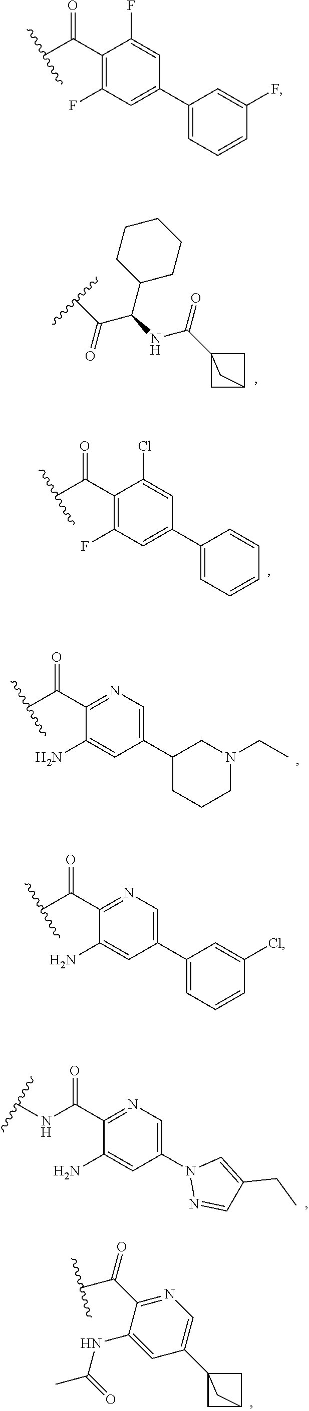

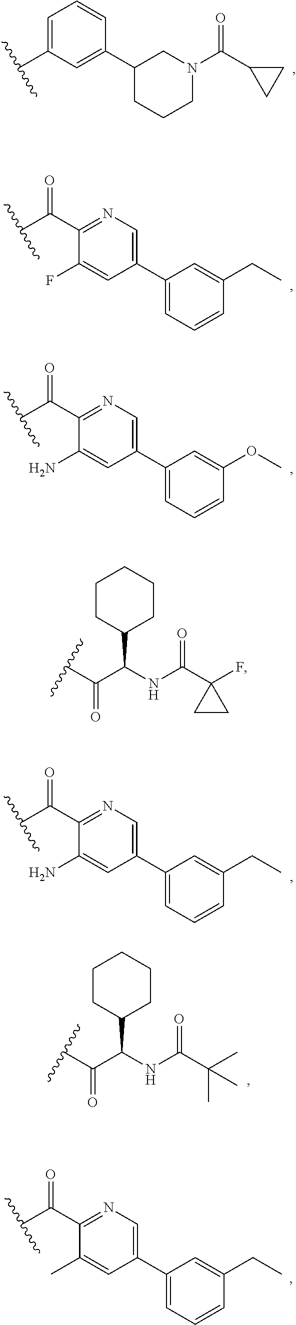

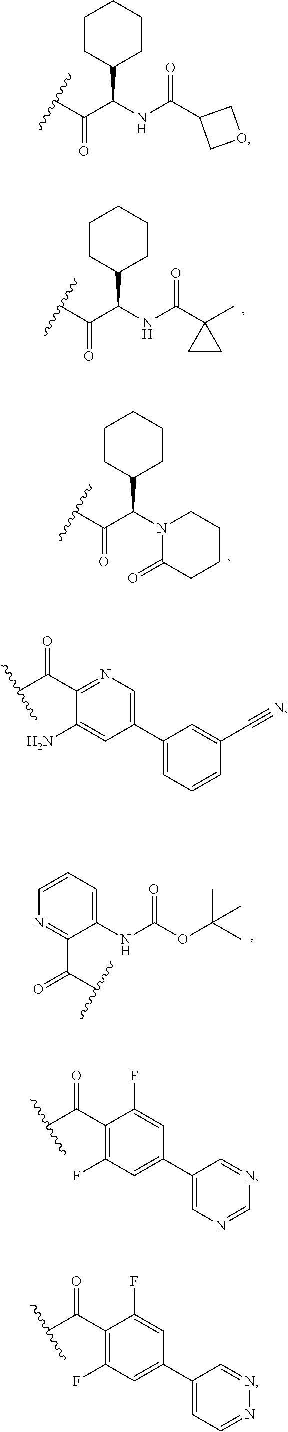

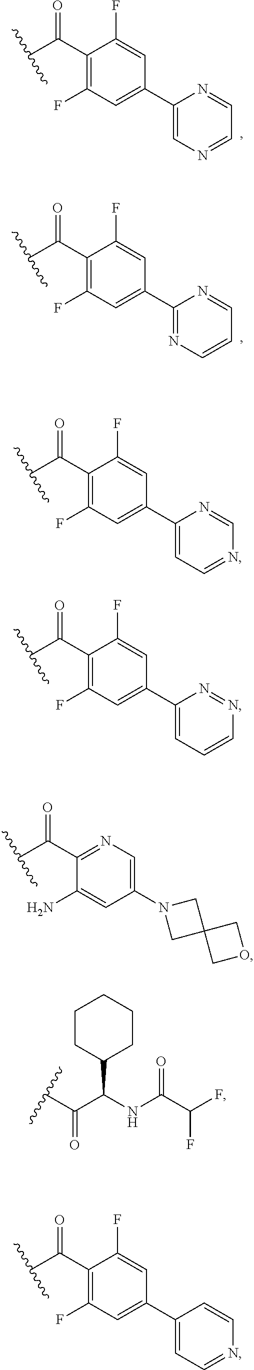

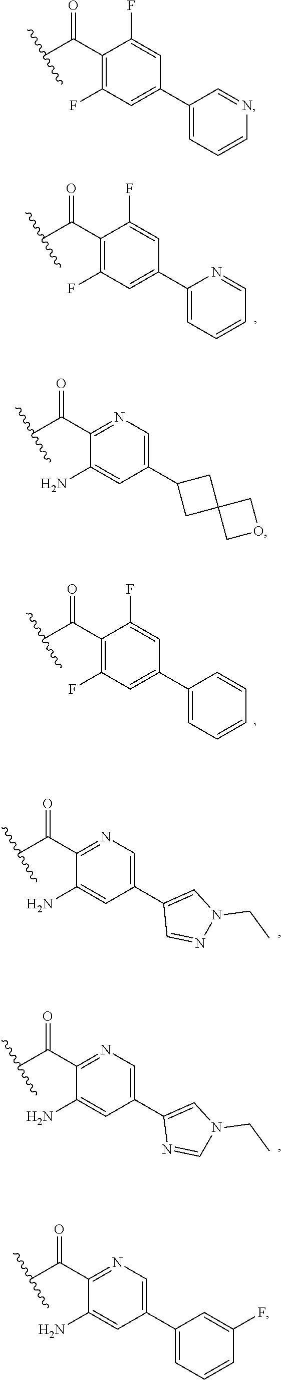

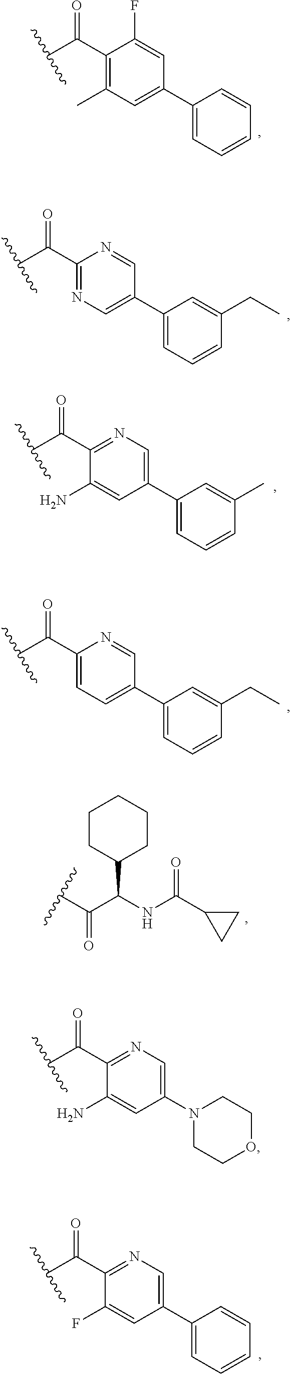

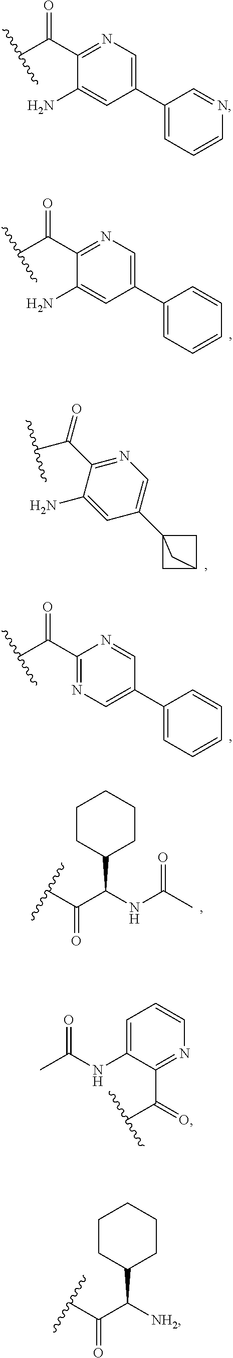

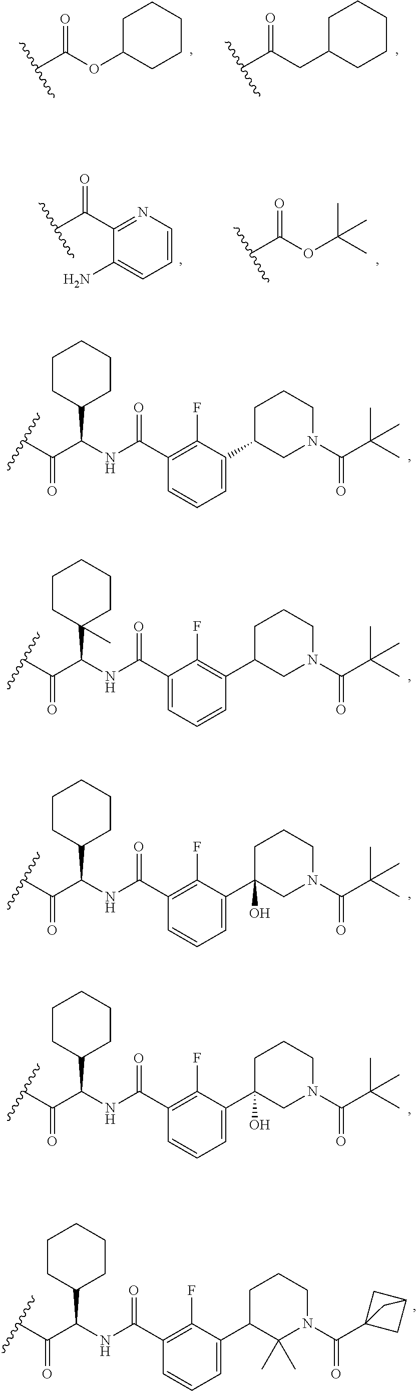

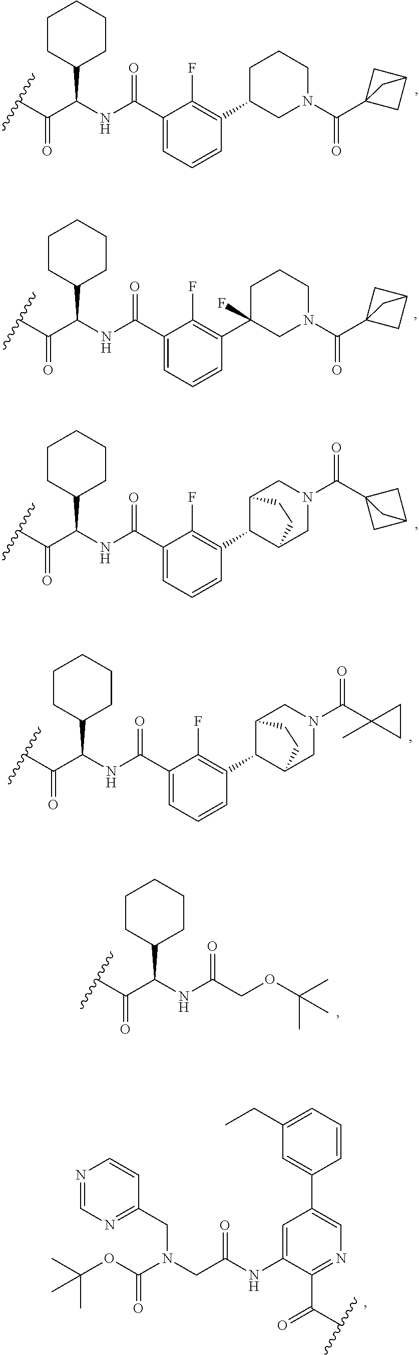 [SMILES: *C(=O)[C@H](NC(=O)c1cccc([C@@H]2CCCN(C(=O)C34CC(C3)C4)C2)c1F)C1CCCCC1 ; *C(=O)[C@H](NC(=O)c1cccc([C@]2(F)CCCN(C(=O)C34CC(C3)C4)C2)c1F)C1CCCCC1 ; *C(=O)[C@H](NC(=O)c1cccc([C@@H]2[C@@H]3CC[C@H]2CN(C(=O)C24CC(C2)C4)C3)c1F)C1CCCCC1 ; *C(=O)[C@H](NC(=O)c1cccc([C@@H]2[C@@H]3CC[C@H]2CN(C(=O)C2(C)CC2)C3)c1F)C1CCCCC1 ; *C(=O)[C@H](NC(=O)COC(C)(C)C)C1CCCCC1 ; *C(=O)c1ncc(-c2cccc(CC)c2)cc1NC(=O)CN(Cc1ccncn1)C(=O)OC(C)(C)C]

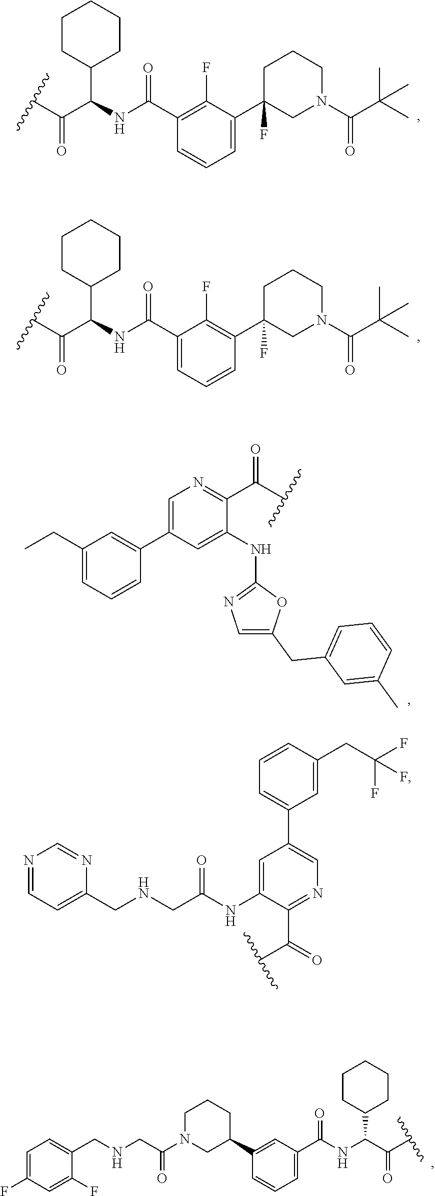

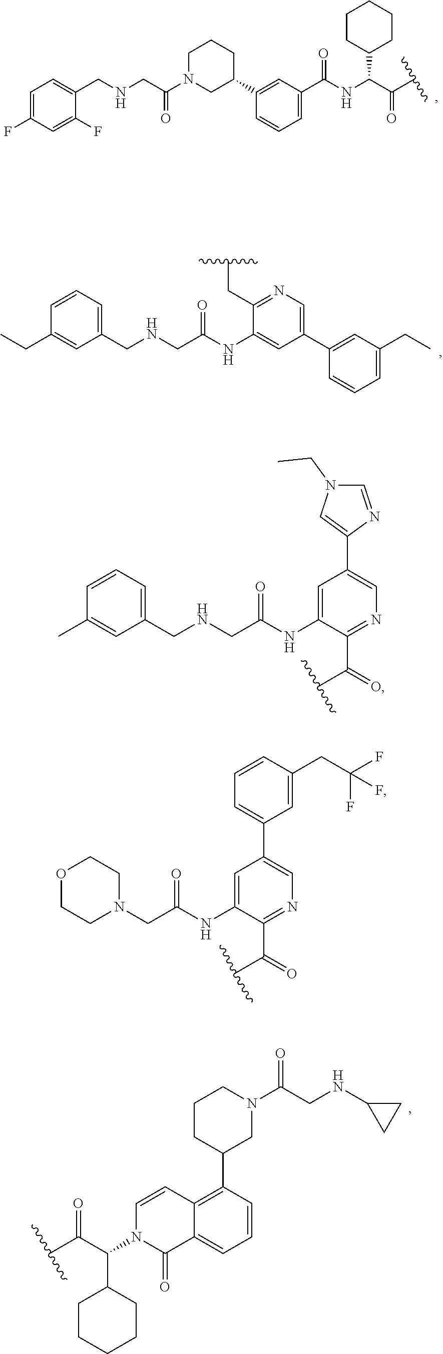

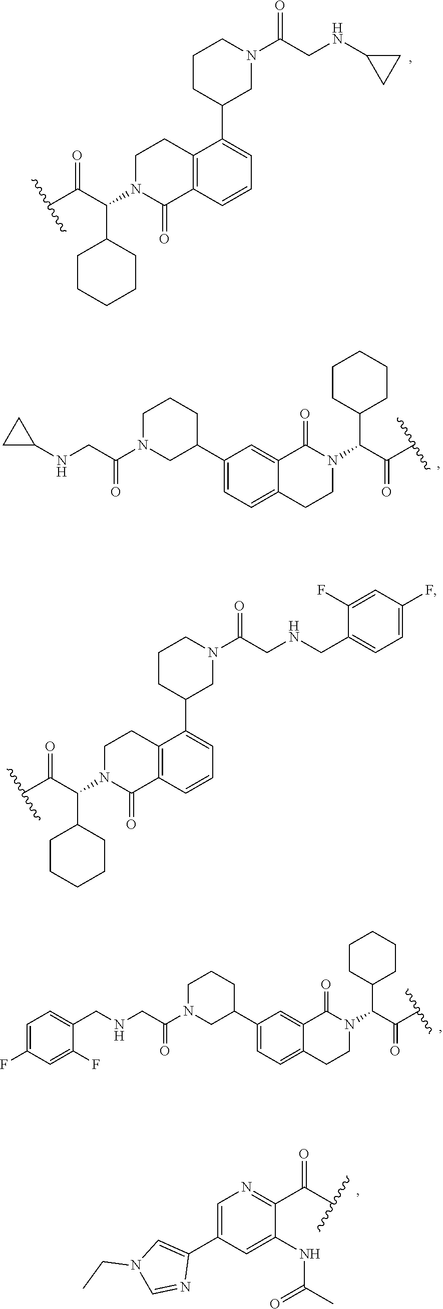 [SMILES: *C(=O)[C@@H](C1CCCCC1)N1CCc2c(cccc2C2CCCN(C(=O)CNC3CC3)C2)C1=O ; *C(=O)[C@@H](C1CCCCC1)N1CCc2ccc(C3CCCN(C(=O)CNC4CC4)C3)cc2C1=O ; *C(=O)[C@@H](C1CCCCC1)N1CCc2c(cccc2C2CCCN(C(=O)CNCc3ccc(F)cc3F)C2)C1=O ; *C(=O)[C@@H](C1CCCCC1)N1CCc2ccc(C3CCCN(C(=O)CNCc4ccc(F)cc4F)C3)cc2C1=O ; *C(=O)c1ncc(-c2cn(CC)cn2)cc1NC(C)=O]

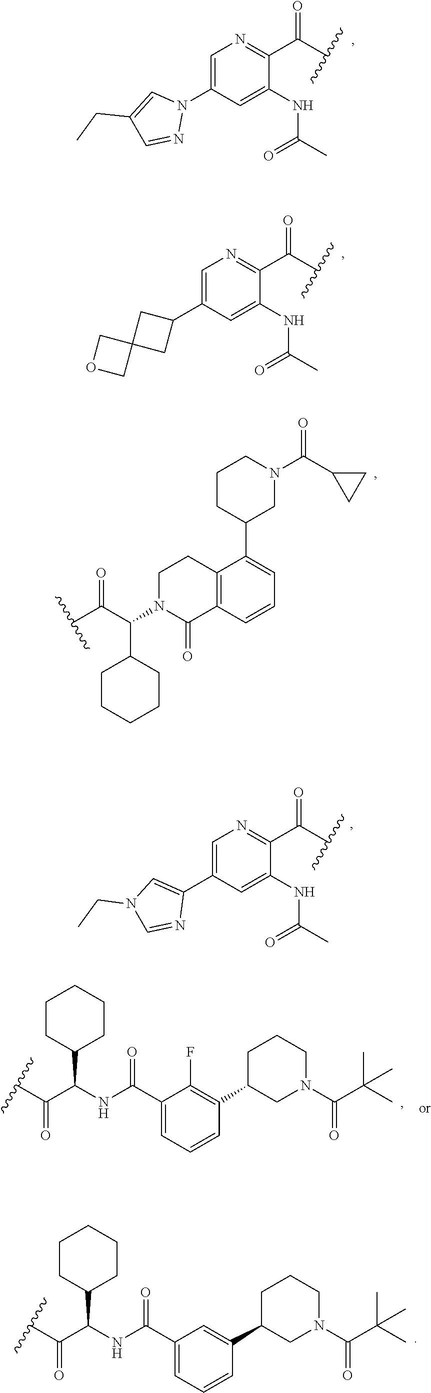

50. The compound of claim 1, wherein L is a covalent bond or a bivalent, saturated or partially unsaturated, straight or branched C₁₋₂₀ hydrocarbon chain, wherein 0-6 methylene units of L are independently replaced by -Cy-, —O—, —NR—, —S—, —OC(O)—, —C(O)O—, —C(O)—, —S(O)—, —S(O)₂—, —N(R)S(O)₂—, —S(O)₂N(R)—, —N(R)C(O)—, —C(O)N(R)—, —OC(O)N(R)—, —N(R)C(O)O—.

51-55. (canceled)

56. The compound of claim 1, wherein the DIM is a DBM selected from those depicted in Table A.

57. The compound of claim 1, wherein L is selected from those depicted in Table B.

58. The compound of claim 1, wherein said compound is selected from any one of the compounds depicted in Table 1, or a pharmaceutically acceptable salt thereof.

59. A pharmaceutical composition comprising a compound of claim 1, and a pharmaceutically acceptable carrier, adjuvant, or vehicle.

60. (canceled)

61. A method of inhibiting PARP in a patient or biological sample comprising administering to said patient, or contacting said biological sample, with a compound according to claim 1, or a pharmaceutical composition thereof.

62. A method of treating a PARP-mediated disorder, disease, or condition in a patient comprising administering to said patient a compound according to claim 1, or a pharmaceutical composition thereof.

63-64. (canceled)
